(19) United States
(12) Patent Application Publication
(10) Pub. No.: US 2006/0003323 A1
(43) Pub. Date: Jan. 5, 2006

(54) Therapeutic polypeptides, nucleic acids encoding same, and methods of use

(76) Inventors: John Alsobrook, Madison, CT (US); Enrique Alvarez, Clinton, CT (US); David Anderson, Plantsville, CT (US); Ferenc Boldog, North Haven, CT (US); Stacie Casman, North Haven, CT (US); Elina Catterton, Milford, CT (US); Andrei Chapoval, Branford, CT (US); Julie Crabtree-Bokor, Gainesville, FL (US); Shlomit Edinger, New Haven, CT (US); Karen Ellerman, Branford, CT (US); Seth Ettenberg, New Haven, CT (US); Esha Gangolli, Madison, CT (US); Valerie Gerlach, Branford, CT (US); Linda Gorman, Branford, CT (US); Erik Gunther, Branford, CT (US); Xiaojia (Sasha) Guo, Branford, CT (US); Vladimir Gusev, Madison, CT (US); John Herrmann, Guilford, CT (US); Weizhen Ji, Branford, CT (US); Ramesh Kekuda, Stamford, CT (US); Li Li, Branford, CT (US); Xiaohong Liu, Branford, CT (US); John MacDougall, Hamden, CT (US); Timothy Machlachlan, Cheshire, CT (US); Uriel Malyankar, North Branford, CT (US); Amanda Mezick, Hamden, CT (US); Isabelle Millet, Milford, CT (US); Vishnu Mishra, Gainesville, FL (US); Muralidhara Padigaru, Branford, CT (US); Meera Patturajan, Branford, CT (US); Carol Pena, New Haven, CT (US); John Peyman, New Haven, CT (US); Debasish Raha, Hamden, CT (US); Luca Rastelli, Guilford, CT (US); Daniel Rieger, Branford, CT (US); Mark Rothenberg, Clinton, CT (US); Paul Sciore, North Haven, CT (US); Suresh Shenoy, Branford, CT (US); Richard Shimkets, Guilford, CT (US); Glennda Smithson, Guilford, CT (US); Kimberly Spytek, Ellington, CT (US); David Stone, Guilford, CT (US); Corine Vernet, Chernex (CH); Edward Voss, Wallingford, CT (US); Mei Zhong, Branford, CT (US); Hiahong Zhong, Guilford, CT (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/453,372

(22) Filed: Jun. 3, 2003

Related U.S. Application Data
(63) Continuation-in-part of application No. 09/789,390, filed on Feb. 23, 2001, now abandoned.
(60) Provisional application No. 60/185,967, filed on Mar. 1, 2000.

(51) Int. Cl. C12Q 1/68

(52) U.S. Cl. 435/6

(57) ABSTRACT

Disclosed herein are nucleic acid sequences that encode novel polypeptides. Also disclosed are polypeptides encoded by these nucleic acid sequences, and antibodies that immunospecifically bind to the polypeptide, as well as derivatives, variants, mutants, or fragments of the novel polypeptide, polynucleotide, or antibody specific to the polypeptide. Vectors, host cells, antibodies and recombinant methods for producing the polypeptides and polynucleotides, as well as methods for using same are also included. The invention further discloses therapeutic, diagnostic and research methods for diagnosis, treatment, and prevention of disorders involving any one of these novel human nucleic acids and proteins.

### RELATED APPLICATIONS

This application is a continuation-in-part of U.S. Ser. No. 09/789,390, filed Feb. 23, 2001, which claims priority to U.S. Ser. No. 60/185,967, filed Mar. 1, 2000; U.S. Ser. No. 09/823,187, filed Mar. 29, 2001, which claims priority to U.S. Ser. No. 60/195,792, filed Mar. 10, 2000; U.S. Ser. No. 09/839,446, filed Mar. 19, 2001, which claims priority to U.S. Ser. No. 60/199,476, filed Mar. 25, 2000; and U.S. Ser. No. 60/199,476, filed Apr. 25, 20001; U.S. Ser. No. 09/863,776, filed May 23, 2001, which claims priority to U.S. Ser. No. 60/208,263, filed May 31, 2000; U.S. Ser. No. 09/939,398, filed Aug. 24, 2001, which claims priority to U.S. Ser. No. 60/227,800, filed Aug. 25, 2000; U.S. Ser. No. 09/964,956, filed Sep. 26, 2001, which claims priority to U.S. Ser. No. 60/236,135, filed Sep. 28, 2000; U.S. Ser. No. 10/015,115, filed Nov. 13, 2001, which claims priority to U.S. Ser. No. 60/248,153, filed Nov. 13, 2000; U.S. Ser. No. 10/028,248, filed Dec. 19, 2001, which claims priority to U.S. Ser. No. 60/285,189, filed Apr. 20, 2001; U.S. Ser. No. 10/032,189, filed Dec. 21, 2001, which claims priority to U.S. Ser. No. 60/258,171, filed Dec. 22, 2000; and U.S. Ser. No. 60/257,495, filed Dec. 21, 2000; U.S. Ser. No. 10/055,877, filed Jan. 22, 2002, which claims priority to U.S. Ser. No. 60/296,960, filed Jun. 8, 2001 and U.S. Ser. No. 60/335,669, filed Oct. 31, 2001; U.S. Ser. No. 10/072,012, filed Jan. 31, 2002, which claims priority to U.S. Ser. No. 60/265,412, filed Jan. 31, 2001; U.S. Ser. No. 10/080,334, filed Feb. 21, 2002, which claims priority to U.S. Ser. No. 60/270,220, filed Feb. 21, 2001; U.S. Ser. No. 10/093,463, filed Mar. 8, 2002, which claims priority to U.S. Ser. No. 60/277,311, filed Mar. 20, 2001; U.S. Ser. No. 10/114,153, filed Apr. 2, 2002, which claims priority to U.S. Ser. No. 60/283,678, filed Apr. 13, 2001; and U.S. Ser. No. 60/286,292, filed Apr. 25, 2001; U.S. Ser. No. 10/114,270, filed Apr. 2, 2002, which claims priority to U.S. Ser. No. 60/281,136, filed Apr. 3, 2001; U.S. Ser. No. 10/138,588, filed Apr. 1, 2002 which claims priority to U.S. Ser. No. 60/288,900, filed May 4, 2001; U.S. Ser. No. 09/783,429, filed Feb. 14, 2001; U.S. Ser. No. 09/800,198, filed Mar. 5, 2001; U.S. Ser. No. 10/023,634, filed Dec. 17, 2001; U.S. Ser. No. 10/038,854, filed Dec. 31, 2001; U.S. Ser. No. 10/051,874, filed Jan. 16, 2002; and U.S. Ser. No. 10/072,013, filed Feb. 8, 2002; and claims the benefit of U.S. Ser. No. 60/386,376, filed Jun. 6, 2002; U.S. Ser. No. 60/385,784, filed Jun. 4, 2002;U.S. Ser. No. 60/386,453, filed Jun. 6, 2002; U.S. Ser. No. 60/402,389, filed Aug. 9, 2002; U.S. Ser. No. 60/412,528, filed Sep. 20, 2002; U.S. Ser. No. 60/387,016, filed Jun. 6, 2002; U.S. Ser. No. 60/386,796, filed Jun. 7, 2002; U.S. Ser. No. 60/386,971, filed Jun. 7, 2002; U.S. Ser. No. 60/386,041, filed Jun. 5, 2002; U.S. Ser. No. 60/387,610, filed Jun. 11, 2002; U.S. Ser. No. 60/386,047, filed Jun. 5, 2002; U.S. Ser. No. 60/387,400, filed Jun. 10, 2002; U.S. Ser. No. 60/402,156, filed Aug. 9, 2002; U.S. Ser. No. 60/386,816, filed Jun. 7, 2002; U.S. Ser. No. 60/402,256, filed Aug. 9, 2002; U.S. Ser. No. 60/389,144, filed Jun. 14, 2002; U.S. Ser. No. 60/386,942, filed Jun. 7, 2002; U.S. Ser. No. 60/387,262, filed Jun. 7, 2002; U.S. Ser. No. 60/389,120, filed Jun. 14, 2002; U.S. Ser. No. 60/421,156, filed Oct. 28, 2002; U.S. Ser. No. 60/423,130, filed Nov. 1, 2002; U.S. Ser. No. 60/425,453, filed Nov. 12, 2002; U.S. Ser. No. 60/386,931, filed Jun. 7, 2002; U.S. Ser. No. 60/403,459, filed Aug. 13, 2002; U.S. Ser. No. 60/387,535, filed Jun. 10, 2002; U.S. Ser. No. 60/403,448, filed Aug. 13, 2002; U.S. Ser. No. 60/423,798, filed Nov. 5, 2002; U.S. Ser. No. 60/387,859, filed Jun. 11, 2002; U.S. Ser. No. 60/402,821, filed Aug. 12, 2002; U.S. Ser. No. 60/417,186, filed Oct. 9, 2002; U.S. Ser. No. 10/015,115, filed Nov. 13, 2001; U.S. Ser. No. 60/387,702, filed Jun. 11, 2002; U.S. Ser. No. 60/387,634, filed Jun. 11, 2002; U. S.S. N. 60/387,934, filed Jun. 12,2002; U.S. Ser. No. 60/387,696, filed Jun. 11, 2002; U.S. Ser. No. 60/390,006, filed Jun. 19, 2002; U.S. Ser. No. 60/387,668, filed Jun. 11, 2002; U.S. Ser. No. 60/402,786, filed Aug. 12, 2002; U.S. Ser. No. 60/410,084, filed Sep. 1 2, 2002 ; U.S. Ser. No. 60/420,639, filed Oct. 23, 2002; U.S. Ser. No. 60/386,864, filed Jun. 6, 2002; U.S. Ser. No. 60/401,628, filed Aug. 6, 2002; U.S. Ser. No. 60/406,182, filed Aug. 26, 2002; U.S. Ser. No. 60/387,836, filed Jun. 11, 2002; U.S. Ser. No. 60/389,729, filed Jun. 17, 2002; U.S. Ser. No. 60/388,022, filed Jun. 12, 2002; U.S. Ser. No. 60/402,816, filed Aug. 12, 2002; U.S. Ser. No. 60/412,731, filed Sep. 23, 2002; U.S. Ser. No. 60/414,954, filed Sep. 30, 2002; U.S. Ser. No. 60 /388,096, filed Jun. 12, 2002; U.S. Ser. No. 60/414,840, filed Sep. 30, 2002; U.S. Ser. No. 60/417,406, filed Oct. 9, 2002; U.S. Ser. No. 60/389,123, filed Jun. 13, 2002; U.S. Ser. No. 60/402,832, filed Aug. 12, 2002; U.S. Ser. No. 60/403,532, filed Aug. 13, 2002; U.S. Ser. No. 60/414,839, filed Sep. 30, 2002; U.S. Ser. No. 60/387,625, filed Jun. 11, 2002; U.S. Ser. No. 60/390,209, filed Jun. 19, 2002; U.S. Ser. No. 60/403,531, filed Aug. 13, 2002; U.S. Ser. No. 60/387,933, filed Jun. 12, 2002; U.S. Ser. No. 60/406,355, filed Aug. 26, 2002; U.S. Ser. No. 10/094,886, filed Mar. 7, 2002; U.S. Ser. No. 60/389,118, filed Jun. 14, 2002; U.S. Ser. No. 60/406,317, filed Aug. 13, 2002; U.S. Ser. No. 60/403,563, filed Aug. 13, 2002; U.S. Ser. No. 60/389,884, filed Jun. 18, 2002; U.S. Ser. No. 60/406,240, filed Aug. 27, 2002; U.S. Ser. No. 60/389,146, filed Jun. 14, 2002; U.S. Ser. No. 60/387,960, filed Jun. 12, 2002; U.S. Ser. No. 60/403,617, filed Aug. 15, 2002; U.S. Ser. No. 60/348,693, filed Jan. 14, 2002; U.S. Ser. No. 60/390,763, filed Jun. 21, 2002; U.S. Ser. No. 60/385,120, filed Jun. 3, 2002; U.S. Ser. No. 60/414,801, filed Sep. 30, 2002; U.S. Ser. No. 60/422,690, filed Oct. 31, 2002; U.S. Ser. No. 60/389,742, filed Jun. 17, 2002; U.S. Ser. No. 60/396,706, filed Jul. 17, 2002.

### FIELD OF THE INVENTION

The present invention relates to novel polypeptides, and the nucleic acids encoding them, having properties related to stimulation of biochemical or physiological responses in a cell, a tissue, an organ or an organism. More particularly, the novel polypeptides are gene products of novel genes, or are specified biologically active fragments or derivatives thereof. Methods of use encompass diagnostic and prognostic assay procedures as well as methods of treating diverse pathological conditions.

### BACKGROUND OF THE INVENTION

Eukaryotic cells are characterized by biochemical and physiological processes which under normal conditions are exquisitely balanced to achieve the preservation and propagation of the cells. When such cells are components of multicellular organisms such as vertebrates, or more particularly organisms such as mammals, the regulation of the biochemical and physiological processes involves intricate signaling pathways. Frequently, such signaling pathways involve extracellular signaling proteins, cellular receptors that bind the signaling proteins, and signal transducing components located within the cells.

Signaling proteins may be classified as endocrine effectors, paracrine effectors or autocrine effectors. Endocrine effectors are signaling molecules secreted by a given organ into the circulatory system, which are then transported to a distant target organ or tissue. The target cells include the receptors for the endocrine effector, and when the endocrine effector binds, a signaling cascade is induced. Paracrine effectors involve secreting cells and receptor cells in close proximity to each other, for example two different classes of cells in the same tissue or organ. One class of cells secretes the paracrine effector, which then reaches the second class of cells, for example by diffusion through the extracellular fluid. The second class of cells contains the receptors for the paracrine effector; binding of the effector results in induction of the signaling cascade that elicits the corresponding biochemical or physiological effect. Autocrine effectors are highly analogous to paracrine effectors, except that the same cell type that secretes the autocrine effector also contains the receptor. Thus the autocrine effector binds to receptors on the same cell, or on identical neighboring cells. The binding process then elicits the characteristic biochemical or physiological effect.

Signaling processes may elicit a variety of effects on cells and tissues including by way of nonlimiting example induction of cell or tissue proliferation, suppression of growth or proliferation, induction of differentiation or maturation of a cell or tissue, and suppression of differentiation or maturation of a cell or tissue.

Many pathological conditions involve dysregulation of expression of important effector proteins. In certain classes of pathologies the dysregulation is manifested as diminished or suppressed level of synthesis and secretion of protein effectors. In other classes of pathologies the dysregulation is manifested as increased or up-regulated level of synthesis and secretion of protein effectors. In a clinical setting a subject may be suspected of suffering from a condition brought on by altered or mis-regulated levels of a protein effector of interest. Therefore there is a need to assay for the level of the protein effector of interest in a biological sample from such a subject, and to compare the level with that characteristic of a nonpathological condition. There also is a need to provide the protein effector as a product of manufacture. Administration of the effector to a subject in need thereof is useful in treatment of the pathological condition. Accordingly, there is a need for a method of treatment of a pathological condition brought on by a diminished or suppressed levels of the protein effector of interest. In addition, there is a need for a method of treatment of a pathological condition brought on by a increased or up-regulated levels of the protein effector of interest.

Antibodies are multichain proteins that bind specifically to a given antigen, and bind poorly, or not at all, to substances deemed not to be cognate antigens. Antibodies are comprised of two short chains termed light chains and two long chains termed heavy chains. These chains are constituted of immunoglobulin domains, of which generally there are two classes: one variable domain per chain, one constant domain in light chains, and three or more constant domains in heavy chains. The antigen-specific portion of the immunoglobulin molecules resides in the variable domains; the variable domains of one light chain and one heavy chain associate with each other to generate the antigen-binding moiety. Antibodies that bind immunospecifically to a cognate or target antigen bind with high affinities. Accordingly, they are useful in assaying specifically for the presence of the antigen in a sample. In addition, they have the potential of inactivating the activity of the antigen.

Therefore there is a need to assay for the level of a protein effector of interest in a biological sample from such a subject, and to compare this level with that characteristic of a nonpathological condition. In particular, there is a need for such an assay based on the use of an antibody that binds immunospecifically to the antigen. There further is a need to inhibit the activity of the protein effector in cases where a pathological condition arises from elevated or excessive levels of the effector based on the use of an antibody that binds immunospecifically to the effector. Thus, there is a need for the antibody as a product of manufacture. There further is a need for a method of treatment of a pathological condition brought on by an elevated or excessive level of the protein effector of interest based on administering the antibody to the subject.

### SUMMARY OF THE INVENTION

The invention is based in part upon the discovery of isolated polypeptides including amino acid sequences selected from mature forms of the amino acid sequences selected from the group consisting of SEQ ID NO:2n, wherein n is an integer between 1 and 606. The novel nucleic acids and polypeptides are referred to herein as NOV1a, NOV1b, NOV1c, NOV1d, NOV2a, NOV2b, NOV2c, NOV2d, NOV3a, NOV3b, etc. These nucleic acids and polypeptides, as well as derivatives, homologs, analogs and fragments thereof, will hereinafter be collectively designated as “NOVX” nucleic acid or polypeptide sequences.

The invention also is based in part upon variants of a mature form of the amino acid sequence selected from the group consisting of SEQ ID NO:2n, wherein n is an integer between 1 and 606, wherein any amino acid in the mature form is changed to a different amino acid, provided that no more than 15% of the amino acid residues in the sequence of the mature form are so changed. In another embodiment, the invention includes the amino acid sequences selected from the group consisting of SEQ ID NO:2n, wherein n is an integer between 1 and 606. In another embodiment, the invention also comprises variants of the amino acid sequence selected from the group consisting of SEQ ID NO:2n, wherein n is an integer between 1 and 606 wherein any amino acid specified in the chosen sequence is changed to a different amino acid, provided that no more than 15% of the amino acid residues in the sequence are so changed. The invention also involves fragments of any of the mature forms of the amino acid sequences selected from the group consisting of SEQ ID NO:2n, wherein n is an integer between 1 and 606, or any other amino acid sequence selected from this group. The invention also comprises fragments from these groups in which up to 15% of the residues are changed.

In another embodiment, the invention encompasses polypeptides that are naturally occurring allelic variants of the sequence selected from the group consisting of SEQ ID NO:2n, wherein n is an integer between 1 and 606. These allelic variants include amino acid sequences that are the translations of nucleic acid sequences differing by a single nucleotide from nucleic acid sequences selected from the group consisting of SEQ ID NOS: 2n−1, wherein n is an integer between 1 and 606. The variant polypeptide where any amino acid changed in the chosen sequence is changed to provide a conservative substitution.

In another embodiment, the invention comprises a pharmaceutical composition involving a polypeptide with an amino acid sequence selected from the group consisting of SEQ ID NO:2n, wherein n is an integer between 1 and 606 and a pharmaceutically acceptable carrier. In another embodiment, the invention involves a kit, including, in one or more containers, this pharmaceutical composition.

In another embodiment, the invention includes the use of a therapeutic in the manufacture of a medicament for treating a syndrome associated with a human disease, the disease being selected from a pathology associated with a polypeptide with an amino acid sequence selected from the group consisting of SEQ ID NO:2n, wherein n is an integer between 1 and 606 wherein said therapeutic is the polypeptide selected from this group.

In another embodiment, the invention comprises a method for determining the presence or amount of a polypeptide with an amino acid sequence selected from the group consisting of SEQ ID NO:2n, wherein n is an integer between 1 and 606 in a sample, the method involving providing the sample; introducing the sample to an antibody that binds immunospecifically to the polypeptide; and determining the presence or amount of antibody bound to the polypeptide, thereby determining the presence or amount of polypeptide in the sample.

In another embodiment, the invention includes a method for determining the presence of or predisposition to a disease associated with altered levels of a polypeptide with an amino acid sequence selected from the group consisting of SEQ ID NO:2n, wherein n is an integer between 1 and 606 in a first mammalian subject, the method involving measuring the level of expression of the polypeptide in a sample from the first mammalian subject; and comparing the amount of the polypeptide in this sample to the amount of the polypeptide present in a control sample from a second mammalian subject known not to have, or not to be predisposed to, the disease, wherein an alteration in the expression level of the polypeptide in the first subject as compared to the control sample indicates the presence of or predisposition to the disease.

In another embodiment, the invention involves a method of identifying an agent that binds to a polypeptide with an amino acid sequence selected from the group consisting of SEQ ID NO:2n, wherein n is an integer between 1 and 606, the method including introducing the polypeptide to the agent; and determining whether the agent binds to the polypeptide. The agent could be a cellular receptor or a downstream effector.

In another embodiment, the invention involves a method for identifying a potential therapeutic agent for use in treatment of a pathology, wherein the pathology is related to aberrant expression or aberrant physiological interactions of a polypeptide with an amino acid sequence selected from the group consisting of SEQ ID NO:2n, wherein n is an integer between 1 and 606, the method including providing a cell expressing the polypeptide of the invention and having a property or function ascribable to the polypeptide; contacting the cell with a composition comprising a candidate substance; and determining whether the substance alters the property or function ascribable to the polypeptide; whereby, if an alteration observed in the presence of the substance is not observed when the cell is contacted with a composition devoid of the substance, the substance is identified as a potential therapeutic agent.

In another embodiment, the invention involves a method forscreening for a modulator of activity or of latency or predisposition to a pathology associated with a polypeptide having an amino acid sequence selected from the group consisting of SEQ ID NO:2n, wherein n is an integer between 1 and 606, the method including administering a test compound to a test animal at increased risk for a pathology associated with the polypeptide of the invention, wherein the test animal recombinantly expresses the polypeptide of the invention; measuring the activity of the polypeptide in the test animal after administering the test compound; and comparing the activity of the protein in the test animal with the activity of the polypeptide in a control animal not administered the polypeptide, wherein a change in the activity of the polypeptide in the test animal relative to the control animal indicates the test compound is a modulator of latency of, or predisposition to,. a pathology associated with the polypeptide of the invention. The recombinant test animal could express a test protein transgene or express the transgene under the control of a promoter at an increased level relative to a wild-type test animal The promoter may or may not b the native gene promoter of the transgene.

In another embodiment, the invention involves a method for modulating the activity of a polypeptide with an amino acid sequence selected from the group consisting of SEQ ID NO:2n, wherein n is an integer between 1 and 606, the method including introducing a cell sample expressing the polypeptide with a compound that binds to the polypeptide in an amount sufficient to modulate the activity of the polypeptide.

In another embodiment, the invention involves a method of treating or preventing a pathology associated with a polypeptide with an amino acid sequence selected from the group consisting of SEQ ID NO:2n, wherein n is an integer between 1 and 606, the method including administering the polypeptide to a subject in which such treatment or prevention is desired in an amount sufficient to treat or prevent the pathology in the subject. The subject could be human.

In another embodiment, the invention involves a method of treating a pathological state in a mammal, the method including administering to the mammal a polypeptide in an amount that is sufficient to alleviate the. pathological state, wherein the polypeptide is a polypeptide having an amino acid sequence at least 95% identical to a polypeptide having the amino acid sequence selected from the group consisting of SEQ ID NO:2n, wherein n is an integer between 1 and 606 or a biologically active fragment thereof.

In another embodiment, the invention involves an isolated nucleic acid molecule comprising a nucleic acid sequence encoding a polypeptide having an amino acid sequence selected from the group consisting of a mature form of the amino acid sequence given SEQ ID NO:2n, wherein n is an integer between 1 and 606; a variant of a mature form of the amino acid sequence selected from the group consisting of SEQ ID NO:2n, wherein n is an integer between 1 and 606 wherein any amino acid in the mature form of the chosen sequence is changed to a different amino acid, provided that no more than 15% of the amino acid residues in the sequence of the mature form are so changed; the amino acid sequence selected from the group consisting of SEQ ID NO:2n, wherein n is an integer between 1 and 606; a variant of the amino acid sequence selected from the group consisting of SEQ ID NO:2n, wherein n is an integer between 1 and 606, in which any amino acid specified in the chosen sequence is changed to a different amino acid, provided that no more than 15% of the amino acid residues in the sequence are so changed; a nucleic acid fragment encoding at least a portion of a polypeptide comprising the amino acid sequence selected from the group consisting of SEQ ID NO:2n, wherein n is an integer between 1 and 606 or any variant of the polypeptide wherein any amino-acid of the chosen sequence is changed to a different amino acid, provided that no more than 10% of the amino acid residues in the sequence are so changed; and the complement of any of the nucleic acid molecules.

In another embodiment, the invention comprises an isolated nucleic acid molecule having a nucleic acid sequence encoding a polypeptide comprising an amino acid sequence selected from the group consisting of a mature form of the amino acid sequence given SEQ ID NO:2n, wherein n is an integer between 1 and 606, wherein the nucleic acid molecule comprises the nucleotide sequence of a naturally occurring allelic nucleic acid variant.

In another embodiment, the invention involves an isolated nucleic acid, molecule including a nucleic acid sequence encoding a polypeptide having an amino acid sequence selected from the group consisting of a mature form of the amino acid sequence given SEQ ID NO:2n, wherein n is an integer between 1 and 606 that encodes a variant polypeptide, wherein the variant polypeptide has the polypeptide sequence of a naturally occurring polypeptide variant.

In another embodiment, the invention comprises an isolated nucleic acid molecule having a nucleic acid sequence encoding a polypeptide comprising an amino acid sequence selected from the group consisting of a mature form of the amino acid sequence given SEQ ID NO:2n, wherein n is an integer between 1 and 606, wherein the nucleic acid molecule differs by a single nucleotide from a nucleic acid sequence selected from the group consisting of SEQ. ID NOS: 2n−1, wherein n is an integer between 1 and 606.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n is an integer between 1 and 606, wherein the nucleic acid molecule comprises a nucleotide sequence selected from the group consisting of the nucleotide sequence selected from the group consisting of SEQ ID NO:2n−1, wherein n is an integer between 1 and 606; a nucleotide sequence wherein one or more nucleotides in the nucleotide sequence selected from the group consisting of SEQ ID NO:2n−1, wherein n is an integer between 1 and 606 is changed from that selected from the group consisting of the chosen sequence to a different nucleotide provided that no more than 15% of the nucleotides are so changed; a nucleic acid fragment of the sequence selected from the group consisting of SEQ ID NO:2n−1, wherein n is an integer between 1 and 606; and a nucleic acid fragment wherein one or more nucleotides in the nucleotide sequence selected from the group consisting of SEQ ID NO:2n−1, wherein n is an integer between 1 and 606 is changed from that selected from the group consisting of the chosen sequence to a different nucleotide provided that no more than 15% of the nucleotides are so changed.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n isan integer between 1 and 606, wherein the nucleic acid molecule hybridizes under stringent conditions to the nucleotide sequence selected from the group consisting of SEQ ID NO:2n−1, wherein n is an integer between 1 and 606, or a complement of the nucleotide sequence.

In another embodiment, the invention includes an isolated nucleic acid molecule having a nucleic acid sequence encoding. a polypeptide including an amino acid sequence selected from the group consisting of a mature form of the amino acid sequence given SEQ ID NO:2n, wherein n is an integer between 1 and 606, wherein the nucleic acid molecule has a nucleotide sequence in which any nucleotide specified in the coding sequence of the chosen nucleotide sequence is changed from that selected from the group consisting of the chosen sequence to a different nucleotide provided that no more than 15% of the nucleotides in the chosen coding sequence are so changed, an isolated second polynucleotide that is a complement of the first polynucleotide, or a fragment of any of them.

In another embodiment, the invention includes a vector involving the nucleic acid molecule having a nucleic acid sequence encoding a polypeptide including an amino acid sequence selected from the group consisting of a mature form of the amino acid sequence given SEQ ID NO:2n, wherein n is an integer between 1 and 606. This vector can have a promoter operably linked to the nucleic acid molecule. This vector can be located within a cell.

In another embodiment, the invention involves a method for determining the presence or amount of a nucleic acid molecule having a nucleic acid sequence encoding a polypeptide including an amino acid sequence selected from the group consisting of a mature form of the amino acid sequence given SEQ ID NO:2n, wherein n is an integer between 1 and 606 in a sample, the method including providing the sample; introducing the sample to a probe that binds to the nucleic acid molecule; and determining the presence or amount of the probe bound to the nucleic acid molecule, thereby determining the presence or amount of the nucleic acid molecule in the sample. The presence or amount of the nucleic acid molecule is used as a marker for cell or tissue type. The cell type can be cancerous.

In another embodiment, the invention involves a method for determining the presence of or predisposition for a disease associated with altered levels of a nucleic acid molecule having a nucleic acid sequence encoding a polypeptide including an amino acid sequence selected from the group consisting of a mature form of the amino acid sequence given SEQ ID NO:2n, wherein n is an integer between 1 and 606 in a first mammalian subject, the method including measuring the amount of the nucleic acid in a sample from the first mammalian subject; and comparing the amount of the nucleic acid in the sample of step (a) to the amount of the nucleic acid present in a control sample from a second mammalian subject known not to have or not be predisposed to, the disease; wherein an alteration in the level of the nucleic acid in the first subject as compared to the control sample indicates the presence of or predisposition to the disease.

The invention further provides an antibody that binds immunospecifically to a NOVX polypeptide. The NOVX antibody may be monoclonal, humanized, or a fully human antibody. Preferably, the antibody has a dissociation constant for the binding of the NOVX polypeptide to the antibody less than 1×10⁻⁹ M. More preferably, the NOVX antibody neutralizes the activity of the NOVX polypeptide.

In a further aspect, the invention provides for the use of a therapeutic in the manufacture of a medicament for treating a syndrome associated with a human disease, associated with a NOVX polypeptide. Preferably the therapeutic is a NOVX antibody.

In yet a further aspect, the invention provides a method of treating or preventing a NOVX-associated disorder, a method of treating a pathological state in a mammal, and a method of treating or preventing a pathology associated with a polypeptide by administering a NOVX antibody to a subject in an amount sufficient to treat or prevent the disorder.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 is a table illustrating the SAGE library data results illustrating overexpression of LIV-1 in breast carcinoma derived cells.

FIG. 2 is is a bar graph showing that antisense knockdown of LIV-1 inhibits breast carcinoma cell line T47D cell growth.

FIG. 3 is is a bar graph showing that antisense knockdown of LwV-1 has less inhibitory effect on Ovcar-4 cell growth.

FIG. 4 is is a bar graph showing RTQPCR validation of knockdown of LIV-1 expression at mRNA level in T47D breast cancer cell lines.

### DETAILED DESCRIPTION OF THE INVENTION

The present invention provides novel nucleotides and polypeptides encoded thereby. Included in the invention are the novel nucleic acid sequences, their encoded polypeptides, antibodies, and other related compounds. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| NOVX | Internal | SEQ ID NO | SEQ ID NO | |
|---|---|---|---|---|
| Assignment | Identification | (nucleic acid) | (amino acid) | Homology |
| NOV1a | CG191083-01 | 1 | 2 | VON WILLEBRAND factor A- related protein homolog - Mus musculus |
| NOV2a | CG191745-01 | 3 | 4 | Integrin alpha-2 precursor (Platelet |
| | | | | membrane glycoprotein Ia) (GPIa) (CD49b) - Homo sapiens |
| NOV3a | CG50253-01 | 5 | 6 | Human secreted protein SCEP-39 |
| NOV3b | 250095765 | 7 | 8 | Human secreted protein SCEP-39 |
| NOV3c | 250095742 | 9 | 10 | Human secreted protein SCEP-39 |
| NOV3d | 250095779 | 11 | 12 | Human secreted protein SCEP-39 |
| NOV3e | 250095794 | 13 | 14 | Human secreted protein SCEP-39 |
| NOV3f | 250095734 | 15 | 16 | Human secreted protein SCEP-39 |
| NOV3g | 250095811 | 17 | 18 | Human secreted protein SCEP-39 |
| NOV3h | 250095799 | 19 | 20 | Human secreted protein SCEP-39 |
| NOV3i | SNP13377609 | 21 | 22 | Human secreted protein SCEP-39 |
| NOV3j | SNP13373929 | 23 | 24 | Human secreted protein SCEP-39 |
| NOV3k | SNP13380272 | 25 | 26 | Human secreted protein SCEP-39 |
| NOV3l | SNP13379745 | 27 | 28 | Human secreted protein SCEP-39 |
| NOV3m | SNP13373930 | 29 | 30 | Human secreted protein SCEP-39 |
| NOV4a | CG50377-04 | 31 | 32 | CSMD1 - Mus musculus |
| NOV4b | CG50377-01 | 33 | 34 | CSMD1 - Mus musculus |
| NOV4c | CG50377-02 | 35 | 36 | CSMD1 - Mus musculus |
| NOV4d | CG50377-03 | 37 | 38 | CSMD1 - Mus musculus |
| NOV4e | CG50377-05 | 39 | 40 | CSMD1 - Mus musculus |
| NOV4f | CG50377-06 | 41 | 42 | CSMD1 - Mus musculus |
| NOV4g | 273095147 | 43 | 44 | CSMD1 - Mus musculus |
| NOV4h | 317459653 | 45 | 46 | CSMD1 - Mus musculus |
| NOV4i | 317459612 | 47 | 48 | CSMD1 - Mus musculus |
| NOV4j | 317286331 | 49 | 50 | CSMD1 - Mus musculus |
| NOV4k | CG50377-07 | 51 | 52 | CSMD1 - Mus musculus |
| NOV4l | CG50377-08 | 53 | 54 | CSMD1 - Mus musculus |
| NOV4m | CG50377-09 | 55 | 56 | CSMD1 - Mus musculus |
| NOV4n | CG50377-10 | 57 | 58 | CSMD1 - Mus musculus |
| NOV4o | CG50377-11 | 59 | 60 | CSMD1 - Mus musculus |
| NOV4p | SNP13382457 | 61 | 62 | CSMD1 - Mus musculus |
| NOV4q | SNP13382458 | 63 | 64 | CSMD1 - Mus musculus |
| NOV5a | CG50389-04 | 65 | 66 | Interleukin 1 receptor-like 2 precursor (IL-1Rrp2) |
| | | | | (IL1R-rp2) - Homo sapiens |
| NOV5b | CG50389-06 | 67 | 68 | Interleukin 1 receptor-like 2 precursor (IL-1Rrp2) |
| | | | | (IL1R-rp2) - Homo sapiens |
| NOV5c | 257448648 | 69 | 70 | Interleukin 1 receptor-like 2 precursor (IL-1Rrp2) |
| | | | | (IL1R-rp2) - Homo sapiens |
| NOV5d | CG50389-01 | 71 | 72 | Interleukin 1 receptor-like 2 precursor (IL-1Rrp2) |
| | | | | (IL1R-rp2) - Homo sapiens |
| NOV5e | CG50389-02 | 73 | 74 | Interleukin 1 receptor-like 2 precursor (IL-1Rrp2) |
| | | | | (IL1R-rp2) - Homo sapiens |
| NOV5f | CG50389-03 | 75 | 76 | Interleukin 1 receptor-like 2 precursor (IL-1Rrp2) |
| | | | | (IL1R-rp2) - Homo sapiens |
| NOV5g | CG50389-05 | 77 | 78 | Interleukin 1 receptor-like 2 precursor (IL-1Rrp2) |
| | | | | (IL1R-rp2) - Homo sapiens |
| NOV5h | SNP13382464 | 79 | 80 | Interleukin 1 receptor-like 2 precursor (IL-1Rrp2) |
| | | | | (IL1R-rp2) - Homo sapiens |
| NOV6a | CG50391-08 | 81 | 82 | Human protease-inhibitor like protein |
| NOV6b | CG50391-09 | 83 | 84 | Human protease-inhibitor like protein |
| NOV6c | CG50391-10 | 85 | 86 | Human protease-inhibitor like protein |
| NOV6d | CG50391-01 | 87 | 88 | Human protease-inhibitor like protein |
| NOV6e | CG50391-02 | 89 | 90 | Human protease-inhibitor like protein |
| NOV6f | CG50391-03 | 91 | 92 | Human protease-inhibitor like protein |
| NOV6g | CG50391-04 | 93 | 94 | Human protease-inhibitor like protein |
| NOV6h | CG50391-05 | 95 | 96 | Human protease-inhibitor like protein |
| NOV6i | CG50391-06 | 97 | 98 | Human protease-inhibitor like protein |
| NOV6j | CG50391-07 | 99 | 100 | Human protease-inhibitor like protein |
| NOV6k | CG50391-11 | 101 | 102 | Human protease-inhibitor like protein |
| NOV6l | SNP13376336 | 103 | 104 | Human protease-inhibitor like protein |
| NOV6m | SNP13376335 | 105 | 106 | Human protease-inhibitor like protein |
| NOV6n | SNP13376334 | 107 | 108 | Human protease-inhibitor like protein |
| NOV6o | SNP13376333 | 109 | 110 | Human protease-inhibitor like protein |
| NOV6p | SNP13382488 | 111 | 112 | Human protease-inhibitor like protein |
| NOV7a | CG50426-17 | 113 | 114 | Ten-m2 - Mus musculus |
| NOV7b | CG50426-21 | 115 | 116 | Ten-m2 - Mus musculus |
| NOV7c | CG50426-15 | 117 | 118 | Ten-m2 - Mus musculus |
| NOV7d | 306276924 | 119 | 120 | Ten-m2 - Mus musculus |
| NOV7e | 306276936 | 121 | 122 | Ten-m2 - Mus musculus |
| NOV7f | 308530526 | 123 | 124 | Ten-m2 - Mus musculus |
| NOV7g | 308530589 | 125 | 126 | Ten-m2 - Mus musculus |
| NOV7h | CG50426-01 | 127 | 128 | Ten-m2 - Mus musculus |
| NOV7i | CG50426-02 | 129 | 130 | Ten-m2 - Mus musculus |
| NOV7j | CG50426-03 | 131 | 132 | Ten-m2 - Mus musculus |
| NOV7k | CG50426-04 | 133 | 134 | Ten-m2 - Mus musculus |
| NOV7l | CG50426-05 | 135 | 136 | Ten-m2 - Mus musculus |
| NOV7m | CG50426-06 | 137 | 138 | Ten-m2 - Mus musculus |
| NOV7n | CG50426-07 | 139 | 140 | Ten-m2 - Mus musculus |
| NOV7o | CG50426-08 | 141 | 142 | Ten-m2 - Mus musculus |
| NOV7p | CG50426-09 | 143 | 144 | Ten-m2 - Mus musculus |
| NOV7q | CG50426-10 | 145 | 146 | Ten-m2 - Mus musculus |
| NOV7r | CG50426-11 | 147 | 148 | Ten-m2 - Mus musculus |
| NOV7s | CG50426-12 | 149 | 150 | Ten-m2 - Mus musculus |
| NOV7t | CG50426-13 | 151 | 152 | Ten-m2 - Mus musculus |
| NOV7u | CG50426-14 | 153 | 154 | Ten-m2 - Mus musculus |
| NOV7v | CG50426-16 | 155 | 156 | Ten-m2 - Mus musculus |
| NOV7w | CG50426-18 | 157 | 158 | Ten-m2 - Mus musculus |
| NOV7x | CG50426-19 | 159 | 160 | Ten-m2 - Mus musculus |
| NOV7y | CG50426-20 | 161 | 162 | Ten-m2 - Mus musculus |
| NOV7z | CG50426-22 | 163 | 164 | Ten-m2 - Mus musculus |
| NOV7a | CG50426-23 | 165 | 166 | Ten-m2 - Mus musculus |
| NOV7b | CG50426-24 | 167 | 168 | Ten-m2 - Mus musculus |
| NOV7c | CG50426-25 | 169 | 170 | Ten-m2 - Mus musculus |
| NOV7d | SNP13375665 | 171 | 172 | Ten-m2 - Mus musculus |
| NOV7e | SNP13375213 | 173 | 174 | Ten-m2 - Mus musculus |
| NOV7f | SNP13374985 | 175 | 176 | Ten-m2 - Mus musculus |
| NOV8a | CG50646-04 | 177 | 178 | Polydom protein precursor - Mus musculus |
| NOV8b | 237582568 | 179 | 180 | Polydom protein precursor - Mus musculus |
| NOV8c | 236589434 | 181 | 182 | Polydom protein precursor - Mus musculus |
| NOV8d | 236495259 | 183 | 184 | Polydom protein precursor - Mus musculus |
| NOV8e | 236495256 | 185 | 186 | Polydom protein precursor - Mus musculus |
| NOV8f | CG50646-01 | 187 | 188 | Polydom protein precursor - Mus musculus |
| NOV8g | CG50646-02 | 189 | 190 | Polydom protein precursor - Mus musculus |
| NOV8h | CG50646-03 | 191 | 192 | Polydom protein precursor - Mus musculus |
| NOV8i | CG50646-05 | 193 | 194 | Polydom protein precursor - Mus musculus |
| NOV8j | SNP13380898 | 195 | 196 | Polydom protein precursor - Mus musculus |
| NOV8k | SNP13380899 | 197 | 198 | Polydom protein precursor - Mus musculus |
| NOV8l | SNP13374702 | 199 | 200 | Polydom protein precursor - Mus musculus |
| NOV8m | SNP13374257 | 201 | 202 | Polydom protein precursor - Mus musculus |
| NOV8n | SNP13382479 | 203 | 204 | Polydom protein precursor - Mus musculus |
| NOV8o | SNP13382480 | 205 | 206 | Polydom protein precursor - Mus musculus |
| NOV9a | CG50736-09 | 207 | 208 | CD44-like precursor FELL - Homo sapiens |
| NOV9b | 197408749 | 209 | 210 | CD44-like precursor FELL - Homo sapiens |
| NOV9c | CG50736-01 | 211 | 212 | CD44-like precursor FELL - Homo sapiens |
| NOV9d | CG50736-02 | 213 | 214 | CD44-like precursor FELL - Homo sapiens |
| NOV9e | CG50736-03 | 215 | 216 | CD44-like precursor FELL - Homo sapiens |
| NOV9f | CG50736-04 | 217 | 218 | CD44-like precursor FELL - Homo sapiens |
| NOV9g | CG50736-05 | 219 | 220 | CD44-like precursor FELL - Homo sapiens |
| NOV9h | CG50736-06 | 221 | 222 | CD44-like precursor FELL - Homo sapiens |
| NOV9i | CG50736-07 | 223 | 224 | CD44-like precursor FELL - Homo sapiens |
| NOV9j | CG50736-08 | 225 | 226 | CD44-like precursor FELL - Homo sapiens |
| NOV9k | CG50736-10 | 227 | 228 | CD44-like precursor FELL - Homo sapiens |
| NOV9l | CG50736-11 | 229 | 230 | CD44-like precursor FELL - Homo sapiens |
| NOV9m | CG50736-12 | 231 | 232 | CD44-like precursor FELL - Homo sapiens |
| NOV9n | CG50736-13 | 233 | 234 | CD44-like precursor FELL - Homo sapiens |
| NOV9o | CG50736-14 | 235 | 236 | CD44-like precursor FELL - Homo sapiens |
| NOV9p | CG50736-15 | 237 | 238 | CD44-like precursor FELL - Homo sapiens |
| NOV9q | CG50736-16 | 239 | 240 | CD44-like precursor FELL - Homo sapiens |
| NOV9r | CG50736-17 | 241 | 242 | CD44-like precursor FELL - Homo sapiens |
| NOV9s | CG50736-18 | 243 | 244 | CD44-like precursor FELL - Homo sapiens |
| NOV9t | CG50736-19 | 245 | 246 | CD44-like precursor FELL - Homo sapiens |
| NOV9u | CG50736-20 | 247 | 248 | CD44-like precursor FELL - Homo sapiens |
| NOV9v | CG50736-21 | 249 | 250 | CD44-like precursor FELL - Homo sapiens |
| NOV9w | CG50736-22 | 251 | 252 | CD44-like precursor FELL - Homo sapiens |
| NOV9x | CG50736-23 | 253 | 254 | CD44-like precursor FELL - Homo sapiens |
| NOV9y | CG50736-24 | 255 | 256 | CD44-like precursor FELL - Homo sapiens |
| NOV10a | CG50925-01 | 257 | 258 | Tumor endothelial marker 5 precursor - Homo sapiens |
| NOV10b | CG50925-08 | 259 | 260 | Tumor endothelial marker 5 precursor - Homo sapiens |
| NOV10c | 226990087 | 261 | 262 | Tumor endothelial marker 5 precursor - Homo sapiens |
| NOV10d | 320054488 | 263 | 264 | Tumor endothelial marker 5 precursor - Homo sapiens |
| NOV10e | CG50925-02 | 265 | 266 | Tumor endothelial marker 5 precursor - Homo sapiens |
| NOV10f | 248208844 | 267 | 268 | Tumor endothelial marker 5 precursor - Homo sapiens |
| NOV10g | CG50925-03 | 269 | 270 | Tumor endothelial marker 5 precursor - Homo sapiens |
| NOV10h | CG50925-04 | 271 | 272 | Tumor endothelial marker 5 precursor - Homo sapiens |
| NOV10i | CG50925-05 | 273 | 274 | Tumor endothelial marker 5 precursor - Homo sapiens |
| NOV10j | CG50925-06 | 275 | 276 | Tumor endothelial marker 5 precursor - Homo sapiens |
| NOV10k | CG50925-07 | 277 | 278 | Tumor endothelial marker 5 precursor - Homo sapiens |
| NOV10l | SNP13380040 | 279 | 280 | Tumor endothelial marker 5 precursor - Homo sapiens |
| NOV11a | CG51027-06 | 281 | 282 | Human immunoglobulin superfamily protein IGSFP-9 |
| NOV11b | 278391205 | 283 | 284 | Human immunoglobulin superfamily protein IGSFP-9 |
| NOV11c | 278391050 | 285 | 286 | Human immunoglobulin superfamily protein IGSFP-9 |
| NOV11d | 277582210 | 287 | 288 | Human immunoglobulin superfamily protein IGSFP-9 |
| NOV11e | 277582173 | 289 | 290 | Human immunoglobulin superfamily protein IGSFP-9 |
| NOV11f | CG51027-09 | 291 | 292 | Human immunoglobulin superfamily protein IGSFP-9 |
| NOV11g | 316763704 | 293 | 294 | Human immunoglobulin superfamily protein IGSFP-9 |
| NOV11h | CG51027-01 | 295 | 296 | Human immunoglobulin superfamily protein IGSFP-9 |
| NOV11i | CG51027-02 | 297 | 298 | Human immunoglobulin superfamily protein IGSFP-9 |
| NOV11j | CG51027-03 | 299 | 300 | Human immunoglobulin superfamily protein IGSFP-9 |
| NOV11k | CG51027-04 | 301 | 302 | Human immunoglobulin superfamily protein IGSFP-9 |
| NOV11l | CG51027-05 | 303 | 304 | Human immunoglobulin superfamily protein IGSFP-9 |
| NOV11m | CG51027-07 | 305 | 306 | Human immunoglobulin superfamily protein IGSFP-9 |
| NOV11n | CG51027-08 | 307 | 308 | Human immunoglobulin superfamily protein IGSFP-9 |
| NOV11o | CG51027-10 | 309 | 310 | Human immunoglobulin superfamily protein IGSFP-9 |
| NOV11p | CG51027-11 | 311 | 312 | Human immunoglobulin superfamily protein IGSFP-9 |
| NOV11q | CG51027-12 | 313 | 314 | Human immunoglobulin superfamily protein IGSFP-9 |
| NOV11r | SNP13376340 | 315 | 316 | Human immunoglobulin superfamily protein IGSFP-9 |
| NOV11s | SNP13376341 | 317 | 318 | Human immunoglobulin superfamily protein IGSFP-9 |
| NOV12a | CG51373-11 | 319 | 320 | Human G protein-coupled receptor |
| NOV12b | CG51373-10 | 321 | 322 | Human G protein-coupled receptor |
| NOV12c | CG51373-08 | 323 | 324 | Human G protein-coupled receptor |
| NOV12d | 237582097 | 325 | 326 | Human G protein-coupled receptor |
| NOV12e | 294385961 | 327 | 328 | Human G protein-coupled receptor |
| NOV12f | 294398021 | 329 | 330 | Human G protein-coupled receptor |
| NOV12g | CG51373-01 | 331 | 332 | Human G protein-coupled receptor |
| NOV12h | CG51373-02 | 333 | 334 | Human G protein-coupled receptor |
| NOV12i | CG51373-03 | 335 | 336 | Human G protein-coupled receptor |
| NOV12j | CG51373-04 | 337 | 338 | Human G protein-coupled receptor |
| NOV12k | CG51373-05 | 339 | 340 | Human G protein-coupled receptor |
| NOV12l | CG51373-06 | 341 | 342 | Human G protein-coupled receptor |
| NOV12m | CG51373-07 | 343 | 344 | Human G protein-coupled receptor |
| NOV12n | CG51373-09 | 345 | 346 | Human G protein-coupled receptor |
| NOV12o | CG51373-12 | 347 | 348 | Human G protein-coupled receptor |
| NOV12p | CG51373-13 | 349 | 350 | Human G protein-coupled receptor |
| NOV12q | CG51373-14 | 351 | 352 | Human G protein-coupled receptor |
| NOV12r | SNP13377352 | 353 | 354 | Human G protein-coupled receptor |
| NOV12s | SNP13379746 | 355 | 356 | Human G protein-coupled receptor |
| NOV12t | SNP13374859 | 357 | 358 | Human G protein-coupled receptor |
| NOV12u | SNP13374858 | 359 | 360 | Human G protein-coupled receptor |
| NOV12v | SNP13374856 | 361 | 362 | Human G protein-coupled receptor |
| NOV12w | SNP13374855 | 363 | 364 | Human G protein-coupled receptor |
| NOV12x | SNP13374854 | 365 | 366 | Human G protein-coupled receptor |
| NOV12y | SNP13374853 | 367 | 368 | Human G protein-coupled receptor |
| NOV13a | CG51622-04 | 369 | 370 | Von Ebner minor protein - Homo sapiens |
| NOV13b | CG51622-03 | 371 | 372 | Von Ebner minor protein - Homo sapiens |
| NOV13c | CG51622-05 | 373 | 374 | Von Ebner minor protein - Homo sapiens |
| NOV13d | CG51622-01 | 375 | 376 | Von Ebner minor protein - Homo sapiens |
| NOV13e | 275698370 | 377 | 378 | Von Ebner minor protein - Homo sapiens |
| NOV13f | 274054187 | 379 | 380 | Von Ebner minor protein - Homo sapiens |
| NOV13g | CG51622-02 | 381 | 382 | Von Ebner minor protein - Homo sapiens |
| NOV13h | CG51622-06 | 383 | 384 | Von Ebner minor protein - Homo sapiens |
| NOV13i | CG51622-07 | 385 | 386 | Von Ebner minor protein - Homo sapiens |
| NOV13j | CG51622-08 | 387 | 388 | Von Ebner minor protein - Homo sapiens |
| NOV13k | CG51622-09 | 389 | 390 | Von Ebner minor protein - Homo sapiens |
| NOV13l | CG51622-10 | 391 | 392 | Von Ebner minor protein - Homo sapiens |
| NOV13m | SNP13375774 | 393 | 394 | Von Ebner minor protein - Homo sapiens |
| NOV13n | SNP13375775 | 395 | 396 | Von Ebner minor protein - Homo sapiens |
| NOV14a | CG51821-01 | 397 | 398 | Sialoadhesin precursor (Sialic acid binding Ig-like lectin-1) |
| | | | | (Siglec- 1) (CD169 antigen) - Homo sapiens |
| NOV14b | 229823091 | 399 | 400 | Sialoadhesin precursor (Sialic acid binding Ig-like lectin-1) |
| | | | | (Siglec- 1) (CD169 antigen) - Homo sapiens |
| NOV14c | 229823107 | 401 | 402 | Sialoadhesin precursor (Sialic acid binding Ig-like lectin-1) |
| | | | | (Siglec- 1) (CD169 antigen) - Homo sapiens |
| NOV14d | 229823095 | 403 | 404 | Sialoadhesin precursor (Sialic acid binding Ig-like lectin-1) |
| | | | | (Siglec- 1) (CD169 antigen) - Homo sapiens |
| NOV14e | CG51821-02 | 405 | 406 | Sialoadhesin precursor (Sialic acid binding Ig-like lectin-1) |
| | | | | (Siglec- 1) (CD169 antigen) - Homo sapiens |
| NOV14f | CG51821-03 | 407 | 408 | Sialoadhesin precursor (Sialic acid binding Ig-like lectin-1) |
| | | | | (Siglec- 1) (CD169 antigen) - Homo sapiens |
| NOV14g | CG51821-04 | 409 | 410 | Sialoadhesin precursor (Sialic acid binding Ig-like lectin-1) |
| | | | | (Siglec- 1) (CD169 antigen) - Homo sapiens |
| NOV14h | SNP13382490 | 411 | 412 | Sialoadhesin precursor (Sialic acid binding Ig-like lectin-1) |
| | | | | (Siglec- 1) (CD169 antigen) - Homo sapiens |
| NOV14i | SNP13382489 | 413 | 414 | Sialoadhesin precursor (Sialic acid binding Ig-like lectin-1) |
| | | | | (Siglec- 1) (CD169 antigen) - Homo sapiens |
| NOV14j | SNP13377616 | 415 | 416 | Sialoadhesin precursor (Sialic acid binding Ig-like lectin-1) |
| | | | | (Siglec- 1) (CD169 antigen) - Homo sapiens |
| NOV14k | SNP13377617 | 417 | 418 | Sialoadhesin precursor (Sialic acid binding Ig-like lectin-1) |
| | | | | (Siglec- 1) (CD169 antigen) - Homo sapiens |
| NOV15a | CG51992-05 | 419 | 420 | CTCL tumor antigen se57-1 - Homo sapiens |
| NOV15b | CG51992-01 | 421 | 422 | CTCL tumor antigen se57-1 - Homo sapiens |
| NOV15c | CG51992-02 | 423 | 424 | CTCL tumor antigen se57-1 - Homo sapiens |
| NOV15d | CG51992-03 | 425 | 426 | CTCL tumor antigen se57-1 - Homo sapiens |
| NOV15e | CG51992-04 | 427 | 428 | CTCL tumor antigen se57-1 - Homo sapiens |
| NOV15f | SNP13382486 | 429 | 430 | CTCL tumor antigen se57-1 - Homo sapiens |
| NOV15g | SNP13382487 | 431 | 432 | CTCL tumor antigen se57-1 - Homo sapiens |
| NOV16a | CG52171-04 | 433 | 434 | Hematopoietic PBX-interacting protein - Homo sapiens |
| NOV16b | CG52171-01 | 435 | 436 | Hematopoietic PBX-interacting protein - Homo sapiens |
| NOV16c | CG52171-02 | 437 | 438 | Hematopoietic PBX-interacting protein - Homo sapiens |
| NOV16d | CG52171-03 | 439 | 440 | Hematopoietic PBX-interacting protein - Homo sapiens |
| NOV16e | CG52171-05 | 441 | 442 | Hematopoietic PBX-interacting protein - Homo sapiens |
| NOV16f | CG52171-06 | 443 | 444 | Hematopoietic PBX-interacting protein - Homo sapiens |
| NOV16g | SNP13374628 | 445 | 446 | Hematopoietic PBX-interacting protein - Homo sapiens |
| NOV16h | SNP13377329 | 447 | 448 | Hematopoietic PBX-interacting protein - Homo sapiens |
| NOV16i | SNP13374627 | 449 | 450 | Hematopoietic PBX-interacting protein - Homo sapiens |
| NOV16j | SNP13374626 | 451 | 452 | Hematopoietic PBX-interacting protein - Homo sapiens |
| NOV16k | SNP13374625 | 453 | 454 | Hematopoietic PBX-interacting protein - Homo sapiens |
| NOV16l | SNP13374624 | 455 | 456 | Hematopoietic PBX-interacting protein - Homo sapiens |
| NOV17a | CG52534-06 | 457 | 458 | Transferrin receptor protein 2 (TfR2) - Homo sapiens |
| NOV17b | CG52534-01 | 459 | 460 | Transferrin receptor protein 2 (TfR2) - Homo sapiens |
| NOV17c | CG52534-02 | 461 | 462 | Transferrin receptor protein 2 (TfR2) - Homo sapiens |
| NOV17d | CG52534-03 | 463 | 464 | Transferrin receptor protein 2 (TfR2) - Homo sapiens |
| NOV17e | CG52534-04 | 465 | 466 | Transferrin receptor protein 2 (TfR2) - Homo sapiens |
| NOV17f | CG52534-05 | 467 | 468 | Transferrin receptor protein 2 (TfR2) - Homo sapiens |
| NOV18a | CG52979-03 | 469 | 470 | G antigen family C 1 protein (Prostate-associated gene protein |
| | | | | 4) (PAGE-4) (PAGE-1) (JM27) (GAGE-9) - Homo sapiens |
| NOV18b | CG52979-01 | 471 | 472 | G antigen family C 1 protein (Prostate-associated gene protein |
| | | | | 4) (PAGE-4) (PAGE-1) (JM27) (GAGE-9) - Homo sapiens |
| NOV18c | CG52979-02 | 473 | 474 | G antigen family C 1 protein (Prostate-associated gene protein |
| | | | | 4) (PAGE-4) (PAGE-1) (JM27) (GAGE-9) - Homo sapiens |
| NOV19a | CG52988-02 | 475 | 476 | G antigen family C 1 protein (Prostate-associated gene protein |
| | | | | 4) (PAGE-4) (PAGE-1) (JM27) (GAGE-9) - Homo sapiens |
| NOV19b | CG52988-03 | 477 | 478 | G antigen family C 1 protein (Prostate-associated gene protein |
| | | | | 4) (PAGE-4) (PAGE-1) (JM27) (GAGE-9) - Homo sapiens |
| NOV19c | CG52988-04 | 479 | 480 | G antigen family C 1 protein (Prostate-associated gene protein |
| | | | | 4) (PAGE-4) (PAGE-1) (JM27) (GAGE-9) - Homo sapiens |
| NOV19d | CG52988-06 | 481 | 482 | G antigen family C 1 protein (Prostate-associated gene protein |
| | | | | 4) (PAGE-4) (PAGE-1) (JM27) (GAGE-9) - Homo sapiens |
| NOV19e | CG52988-07 | 483 | 484 | G antigen family C 1 protein (Prostate-associated gene protein |
| | | | | 4) (PAGE-4) (PAGE-1) (JM27) (GAGE-9) - Homo sapiens |
| NOV19f | CG52988-01 | 485 | 486 | G antigen family C 1 protein (Prostate-associated gene protein |
| | | | | 4) (PAGE-4) (PAGE-1) (JM27) (GAGE-9) - Homo sapiens |
| NOV19g | CG52988-05 | 487 | 488 | G antigen family C 1 protein (Prostate-associated gene protein |
| | | | | 4) (PAGE-4) (PAGE-1) (JM27) (GAGE-9) - Homo sapiens |
| NOV20a | CG53449-04 | 489 | 490 | Butyrophilin like receptor - Homo sapiens |
| NOV20b | CG53449-03 | 491 | 492 | Butyrophilin like receptor - Homo sapiens |
| NOV20c | CG53449-01 | 493 | 494 | Butyrophilin like receptor - Homo sapiens |
| NOV20d | CG53449-02 | 495 | 496 | Butyrophilin like receptor - Homo sapiens |
| NOV20e | CG53449-05 | 497 | 498 | Butyrophilin like receptor - Homo sapiens |
| NOV20f | CG53449-06 | 499 | 500 | Butyrophilin like receptor - Homo sapiens |
| NOV20g | SNP13382434 | 501 | 502 | Butyrophilin like receptor - Homo sapiens |
| NOV20h | SNP13382441 | 503 | 504 | Butyrophilin like receptor - Homo sapiens |
| NOV21a | CG53908-01 | 505 | 506 | Netrin receptor Unc5h1 - Mus musculus |
| NOV21b | CG53908-02 | 507 | 508 | Netrin receptor Unc5h1 - Mus musculus |
| NOV21c | 306075989 | 509 | 510 | Netrin receptor Unc5h1 - Mus musculus |
| NOV21d | CG53908-03 | 511 | 512 | Netrin receptor Unc5h1 - Mus musculus |
| NOV21e | CG53908-04 | 513 | 514 | Netrin receptor Unc5h1 - Mus musculus |
| NOV21f | CG53908-05 | 515 | 516 | Netrin receptor Unc5h1 - Mus musculus |
| NOV21g | CG53908-06 | 517 | 518 | Netrin receptor Unc5h1 - Mus musculus |
| NOV21h | CG53908-07 | 519 | 520 | Netrin receptor Unc5h1 - Mus musculus |
| NOV21i | SNP13382444 | 521 | 522 | Netrin receptor Unc5h1 - Mus musculus |
| NOV21j | SNP13375491 | 523 | 524 | Netrin receptor Unc5h1 - Mus musculus |
| NOV21k | SNP13375492 | 525 | 526 | Netrin receptor Unc5h1 - Mus musculus |
| NOV21l | SNP13375493 | 527 | 528 | Netrin receptor Unc5h1 - Mus musculus |
| NOV21m | SNP13375135 | 529 | 530 | Netrin receptor Unc5h1 - Mus musculus |
| NOV21n | SNP13375136 | 531 | 532 | Netrin receptor Unc5h1 - Mus musculus |
| NOV21o | SNP13375137 | 533 | 534 | Netrin receptor Unc5h1 - Mus musculus |
| NOV21p | SNP13375138 | 535 | 536 | Netrin receptor Unc5h1 - Mus musculus |
| NOV21q | SNP13375494 | 537 | 538 | Netrin receptor Unc5h1 - Mus musculus |
| NOV21r | SNP13375495 | 539 | 540 | Netrin receptor Unc5h1 - Mus musculus |
| NOV21s | SNP13375140 | 541 | 542 | Netrin receptor Unc5h1 - Mus musculus |
| NOV21t | SNP13375139 | 543 | 544 | Netrin receptor Unc5h1 - Mus musculus |
| NOV21u | SNP13375496 | 545 | 546 | Netrin receptor Unc5h1 - Mus musculus |
| NOV22a | CG53944-02 | 547 | 548 | human secreted polypeptide - Homo sapiens |
| NOV22b | CG53944-01 | 549 | 550 | human secreted polypeptide - Homo sapiens |
| NOV23a | CG54308-04 | 551 | 552 | Serpin B12 - Homo sapiens |
| NOV23b | CG54308-01 | 553 | 554 | Serpin B12 - Homo sapiens |
| NOV23c | CG54308-02 | 555 | 556 | Serpin B12 - Homo sapiens |
| NOV23d | CG54308-03 | 557 | 558 | Serpin B12 - Homo sapiens |
| NOV23e | CG54308-05 | 559 | 560 | Serpin B12 - Homo sapiens |
| NOV24a | CG54764-02 | 561 | 562 | Calgizzarin (Endothelial monocyte-activating |
| | | | | polypeptide) (EMAP) - Mus musculus |
| NOV24b | CG54764-01 | 563 | 564 | Calgizzarin (Endothelial monocyte-activating |
| | | | | polypeptide) (EMAP) - Mus musculus |
| NOV25a | CG55033-04 | 565 | 566 | Human toll like receptor like molecule 5 (TLR-L5) |
| NOV25b | CG55033-05 | 567 | 568 | Human toll like receptor like molecule 5 (TLR-L5) |
| NOV25c | CG55033-01 | 569 | 570 | Human toll like receptor like molecule 5 (TLR-L5) |
| NOV25d | 237376826 | 571 | 572 | Human toll like receptor like molecule 5 (TLR-L5) |
| NOV25e | 310658403 | 573 | 574 | Human toll like receptor like molecule 5 (TLR-L5) |
| NOV25f | 317980876 | 575 | 576 | Human toll like receptor like molecule 5 (TLR-L5) |
| NOV25g | 318018151 | 577 | 578 | Human toll like receptor like molecule 5 (TLR-L5) |
| NOV25h | 318176316 | 579 | 580 | Human toll like receptor like molecule 5 (TLR-L5) |
| NOV25i | CG55033-02 | 581 | 582 | Human toll like receptor like molecule 5 (TLR-L5) |
| NOV25j | CG55033-03 | 583 | 584 | Human toll like receptor like molecule 5 (TLR-L5) |
| NOV25k | CG55033-06 | 585 | 586 | Human toll like receptor like molecule 5 (TLR-L5) |
| NOV25l | CG55033-07 | 587 | 588 | Human toll like receptor like molecule 5 (TLR-L5) |
| NOV25m | CG55033-08 | 589 | 590 | Human toll like receptor like molecule 5 (TLR-L5) |
| NOV25n | CG55033-09 | 591 | 592 | Human toll like receptor like molecule 5 (TLR-L5) |
| NOV25o | CG55033-10 | 593 | 594 | Human toll like receptor like molecule 5 (TLR-L5) |
| NOV25p | CG55033-11 | 595 | 596 | Human toll like receptor like molecule 5 (TLR-L5) |
| NOV25q | CG55033-12 | 597 | 598 | Human toll like receptor like molecule 5 (TLR-L5) |
| NOV25r | CG55033-13 | 599 | 600 | Human toll like receptor like molecule 5 (TLR-L5) |
| NOV25s | CG55033-14 | 601 | 602 | Human toll like receptor like molecule 5 (TLR-L5) |
| NOV26a | CG55117-04 | 603 | 604 | Prominin-like protein 1 precursor |
| | | | | (Antigen AC133) (CD133 antigen) - Homo sapiens |
| NOV26b | CG55117-01 | 605 | 606 | Prominin-like protein 1 precursor |
| | | | | (Antigen AC133) (CD133 antigen) - Homo sapiens |
| NOV26c | CG55117-02 | 607 | 608 | Prominin-like protein 1 precursor |
| | | | | (Antigen AC133) (CD133 antigen) - Homo sapiens |
| NOV26d | CG55117-03 | 609 | 610 | Prominin-like protein 1 precursor |
| | | | | (Antigen AC133) (CD133 antigen) - Homo sapiens |
| NOV26e | CG55117-05 | 611 | 612 | Prominin-like protein 1 precursor |
| | | | | (Antigen AC133) (CD133 antigen) - Homo sapiens |
| NOV26f | SNP13382440 | 613 | 614 | Prominin-like protein 1 precursor |
| | | | | (Antigen AC133) (CD133 antigen) - Homo sapiens |
| NOV26g | SNP13382439 | 615 | 616 | Prominin-like protein 1 precursor |
| | | | | (Antigen AC133) (CD133 antigen) - Homo sapiens |
| NOV27a | CG55193-04 | 617 | 618 | PLVAP protein - Homo sapiens (Human), 455 aa |
| NOV27b | 214575880 | 619 | 620 | PLVAP protein - Homo sapiens (Human), 455 aa |
| NOV27c | 214458684 | 621 | 622 | PLVAP protein - Homo sapiens (Human), 455 aa |
| NOV27d | 214458688 | 623 | 624 | PLVAP protein - Homo sapiens (Human), 455 aa |
| NOV27e | CG55193-01 | 625 | 626 | PLVAP protein - Homo sapiens (Human), 455 aa |
| NOV27f | CG55193-02 | 627 | 628 | PLVAP protein - Homo sapiens (Human), 455 aa |
| NOV27g | CG55193-03 | 629 | 630 | PLVAP protein - Homo sapiens (Human), 455 aa |
| NOV27h | CG55193-05 | 631 | 632 | PLVAP protein - Homo sapiens (Human), 455 aa |
| NOV27i | CG55193-06 | 633 | 634 | PLVAP protein - Homo sapiens (Human), 455 aa |
| NOV27j | CG55193-07 | 635 | 636 | PLVAP protein - Homo sapiens (Human), 455 aa |
| NOV28a | CG55256-07 | 637 | 638 | Apolipoprotein E receptor 2 precursor - Homo sapiens |
| NOV28b | CG55256-10 | 639 | 640 | Apolipoprotein E receptor 2 precursor - Homo sapiens |
| NOV28c | 272511714 | 641 | 642 | Apolipoprotein E receptor 2 precursor - Homo sapiens |
| NOV28d | CG55256-01 | 643 | 644 | Apolipoprotein E receptor 2 precursor - Homo sapiens |
| NOV28e | CG55256-02 | 645 | 646 | Apolipoprotein E receptor 2 precursor - Homo sapiens |
| NOV28f | CG55256-03 | 647 | 648 | Apolipoprotein E receptor 2 precursor - Homo sapiens |
| NOV28g | CG55256-04 | 649 | 650 | Apolipoprotein E receptor 2 precursor - Homo sapiens |
| NOV28h | CG55256-05 | 621 | 652 | Apolipoprotein E receptor 2 precursor - Homo sapiens |
| NOV28i | CG55256-06 | 653 | 654 | Apolipoprotein E receptor 2 precursor - Homo sapiens |
| NOV28j | CG55256-08 | 655 | 656 | Apolipoprotein E receptor 2 precursor - Homo sapiens |
| NOV28k | CG55256-09 | 657 | 658 | Apolipoprotein E receptor 2 precursor - Homo sapiens |
| NOV28l | CG55256-11 | 659 | 660 | Apolipoprotein E receptor 2 precursor - Homo sapiens |
| NOV28m | SNP13382497 | 661 | 662 | Apolipoprotein E receptor 2 precursor - Homo sapiens |
| NOV28n | SNP13382498 | 663 | 664 | Apolipoprotein E receptor 2 precursor - Homo sapiens |
| NOV29a | CG55776-01 | 665 | 666 | Human osteoclast protein (OCP) |
| NOV29b | 248210167 | 667 | 668 | Human osteoclast protein (OCP) |
| NOV29c | 247679561 | 669 | 670 | Human osteoclast protein (OCP) |
| NOV29d | 248057904 | 671 | 672 | Human osteoclast protein (OCP) |
| NOV29e | 248057927 | 673 | 674 | Human osteoclast protein (OCP) |
| NOV29f | 249239821 | 675 | 676 | Human osteoclast protein (OCP) |
| NOV29g | 248057920 | 677 | 678 | Human osteoclast protein (OCP) |
| NOV29h | 247679541 | 679 | 680 | Human osteoclast protein (OCP) |
| NOV29i | 249116954 | 681 | 682 | Human osteoclast protein (OCP) |
| NOV29j | 248210155 | 683 | 684 | Human osteoclast protein (OCP) |
| NOV29k | 248213764 | 685 | 686 | Human osteoclast protein (OCP) |
| NOV29l | 248213768 | 687 | 688 | Human osteoclast protein (OCP) |
| NOV29m | 248213772 | 689 | 690 | Human osteoclast protein (OCP) |
| NOV29n | 248586774 | 691 | 692 | Human osteoclast protein (OCP) |
| NOV29o | 248586793 | 693 | 694 | Human osteoclast protein (OCP) |
| NOV29p | 248586820 | 695 | 696 | Human osteoclast protein (OCP) |
| NOV29q | 248586824 | 697 | 698 | Human osteoclast protein (OCP) |
| NOV29r | 247679817 | 699 | 700 | Human osteoclast protein (OCP) |
| NOV29s | 248210264 | 701 | 702 | Human osteoclast protein (OCP) |
| NOV29t | 248210551 | 703 | 704 | Human osteoclast protein (OCP) |
| NOV29u | 248210824 | 705 | 706 | Human osteoclast protein (OCP) |
| NOV29v | 248679541 | 707 | 708 | Human osteoclast protein (OCP) |
| NOV29w | 247679454 | 709 | 710 | Human osteoclast protein (OCP) |
| NOV29x | 314361407 | 711 | 712 | Human osteoclast protein (OCP) |
| NOV29y | 317803448 | 713 | 714 | Human osteoclast protein (OCP) |
| NOV29z | CG55776-02 | 715 | 716 | Human osteoclast protein (OCP) |
| NOV29aa | CG55776-03 | 717 | 718 | Human osteoclast protein (OCP) |
| NOV29ab | CG55776-04 | 719 | 720 | Human osteoclast protein (OCP) |
| NOV29ac | CG55776-05 | 721 | 722 | Human osteoclast protein (OCP) |
| NOV29ad | CG55776-06 | 723 | 724 | Human osteoclast protein (OCP) |
| NOV29ae | CG55776-07 | 725 | 726 | Human osteoclast protein (OCP) |
| NOV29af | CG55776-08 | 727 | 728 | Human osteoclast protein (OCP) |
| NOV29ag | CG55776-09 | 729 | 730 | Human osteoclast protein (OCP) |
| NOV29ah | SNP13376522 | 731 | 732 | Human osteoclast protein (OCP) |
| NOV29ai | SNP13377624 | 733 | 734 | Human osteoclast protein (OCP) |
| NOV29aj | SNP13377625 | 735 | 736 | Human osteoclast protein (OCP) |
| NOV29ak | SNP13376523 | 737 | 738 | Human osteoclast protein (OCP) |
| NOV29al | SNP13376524 | 739 | 740 | Human osteoclast protein (OCP) |
| NOV29am | SNP13376525 | 741 | 742 | Human osteoclast protein (OCP) |
| NOV29an | SNP13376526 | 743 | 744 | Human osteoclast protein (OCP) |
| NOV29ao | SNP13377626 | 745 | 746 | Human osteoclast protein (OCP) |
| NOV29ap | SNP13377641 | 747 | 748 | Human osteoclast protein (OCP) |
| NOV29aq | SNP13377640 | 749 | 750 | Human osteoclast protein (OCP) |
| NOV30a | CG55784-03 | 751 | 752 | NPEH2 - Homo sapiens |
| NOV30b | CG55784-01 | 753 | 754 | NPEH2 - Homo sapiens |
| NOV30c | 312000579 | 755 | 756 | NPEH2 - Homo sapiens |
| NOV30d | 311333341 | 757 | 758 | NPEH2 - Homo sapiens |
| NOV30e | 311333338 | 759 | 760 | NPEH2 - Homo sapiens |
| NOV30f | 311346885 | 761 | 762 | NPEH2 - Homo sapiens |
| NOV30g | 311333299 | 763 | 764 | NPEH2 - Homo sapiens |
| NOV30h | CG55784-02 | 765 | 766 | NPEH2 - Homo sapiens |
| NOV30i | SNP13376439 | 767 | 768 | NPEH2 - Homo sapiens |
| NOV31a | CG55790-02 | 769 | 770 | ICOS ligand precursor (B7 homolog 2) (B7-H2) (B7-like |
| | | | | protein G150) (B7-related protein-1) (B7RP-1) - Homo sapiens |
| NOV31b | 258668431 | 771 | 772 | ICOS ligand precursor (B7 homolog 2) (B7-H2) (B7-like |
| | | | | protein G150) (B7-related protein-1) (B7RP-1) - Homo sapiens |
| NOV31c | 309303509 | 773 | 774 | ICOS ligand precursor (B7 homolog 2) (B7-H2) (B7-like |
| | | | | protein G150) (B7-related protein-1) (B7RP-1) - Homo sapiens |
| NOV31d | 315925314 | 775 | 776 | ICOS ligand precursor (B7 homolog 2) (B7-H2) (B7-like |
| | | | | protein G150) (B7-related protein-1) (B7RP-1) - Homo sapiens |
| NOV31e | 315970230 | 777 | 778 | ICOS ligand precursor (B7 homolog 2) (B7-H2) (B7-like |
| | | | | protein G150) (B7-related protein-1) (B7RP-1) - Homo sapiens |
| NOV31f | CG55790-01 | 779 | 780 | ICOS ligand precursor (B7 homolog 2) (B7-H2) (B7-like |
| | | | | protein G150) (B7-related protein-1) (B7RP-1) - Homo sapiens |
| NOV31g | CG55790-03 | 781 | 782 | ICOS ligand precursor (B7 homolog 2) (B7-H2) (B7-like |
| | | | | protein G150) (B7-related protein-1) (B7RP-1) - Homo sapiens |
| NOV31h | CG55790-04 | 783 | 784 | ICOS ligand precursor (B7 homolog 2) (B7-H2) (B7-like |
| | | | | protein G150) (B7-related protein-1) (B7RP-1) - Homo sapiens |
| NOV31i | CG55790-05 | 785 | 786 | ICOS ligand precursor (B7 homolog 2) (B7-H2) (B7-like |
| | | | | protein G150) (B7-related protein-1) (B7RP-1) - Homo sapiens |
| NOV31j | CG55790-06 | 787 | 788 | ICOS ligand precursor (B7 homolog 2) (B7-H2) (B7-like |
| | | | | protein G150) (B7-related protein-1) (B7RP-1) - Homo sapiens |
| NOV31k | CQ55790-07 | 789 | 790 | ICOS ligand precursor (B7 homolog 2) (B7-H2) (B7-like |
| | | | | protein G150) (B7-related protein-1) (B7RP-1) - Homo sapiens |
| NOV31l | SNP13374852 | 791 | 792 | ICOS ligand precursor (B7 homolog 2) (B7-H2) (B7-like |
| | | | | protein G150) (B7-related protein-1) (B7RP-1) - Homo sapiens |
| NOV32a | CG55906-04 | 793 | 794 | Human breast tumour-associated protein 37 |
| NOV32b | CG55906-05 | 795 | 796 | Human breast tumour-associated protein 37 |
| NOV32c | CG55906-01 | 797 | 798 | Human breast tumour-associated protein 37 |
| NOV32d | 277901794 | 799 | 800 | Human breast tumour-associated protein 37 |
| NOV32e | CG55906-02 | 801 | 802 | Human breast tumour-associated protein 37 |
| NOV32f | 230272941 | 803 | 804 | Human breast tumour-associated protein 37 |
| NOV32g | CG55906-03 | 805 | 806 | Human breast tumour-associated protein 37 |
| NOV32h | SNP13382505 | 807 | 808 | Human breast tumour-associated protein 37 |
| NOV32i | SNP13382504 | 809 | 810 | Human breast tumour-associated protein 37 |
| NOV32j | SNP13376440 | 811 | 812 | Human breast tumour-associated protein 37 |
| NOV33a | CG55908-01 | 813 | 814 | integrin alpha-7 chain precursor - human |
| NOV33b | 253116407 | 815 | 816 | integrin alpha-7 chain precursor - human |
| NOV33c | 253116412 | 817 | 818 | integrin alpha-7 chain precursor - human |
| NOV34a | CG56077-06 | 819 | 820 | LGI4 (Leucine-rich glioma inactivated protein 4) |
| | | | | (LGI1-like protein 3) - Homo sapiens |
| NOV34b | CG56077-01 | 821 | 822 | LGI4 (Leucine-rich glioma inactivated protein 4) |
| | | | | (LGI1-like protein 3) - Homo sapiens |
| NOV34c | CG56077-02 | 823 | 824 | LGI4 (Leucine-rich glioma inactivated protein 4) |
| | | | | (LGI1-like protein 3) - Homo sapiens |
| NOV34d | CG56077-03 | 825 | 826 | LGI4 (Leucine-rich glioma inactivated protein 4) |
| | | | | (LGI1-like protein 3) - Homo sapiens |
| NOV34e | CG56077-04 | 827 | 828 | LGI4 (Leucine-rich glioma inactivated protein 4) |
| | | | | (LGI1-like protein 3) - Homo sapiens |
| NOV34f | CG56077-05 | 829 | 830 | LGI4 (Leucine-rich glioma inactivated protein 4) |
| | | | | (LGI1-like protein 3) - Homo sapiens |
| NOV34g | SNP13374715 | 831 | 832 | LGI4 (Leucine-rich glioma inactivated protein 4) |
| | | | | (LGI1-like protein 3) - Homo sapiens |
| NOV34h | SNP13374714 | 833 | 834 | LGI4 (Leucine-rich glioma inactivated protein 4) |
| | | | | (LGI1-like protein 3) - Homo sapiens |
| NOV35a | CG56110-03 | 835 | 836 | B7-H1 (PD-1-ligand precursor) - Homo sapiens |
| NOV35b | CG56110-07 | 837 | 838 | B7-H1 (PD-1-ligand precursor) - Homo sapiens |
| NOV35c | 274082305 | 839 | 840 | B7-H1 (PD-1-ligand precursor) - Homo sapiens |
| NOV35d | CG56110-01 | 841 | 842 | B7-H1 (PD-1-ligand precursor) - Homo sapiens |
| NOV35e | CG56110-02 | 843 | 844 | B7-H1 (PD-1-ligand precursor) - Homo sapiens |
| NOV35f | CG56110-04 | 845 | 846 | B7-H1 (PD-1-ligand precursor) - Homo sapiens |
| NOV35g | CG56110-05 | 847 | 848 | B7-H1 (PD-1-ligand precursor) - Homo sapiens |
| NOV35h | CG56110-06 | 849 | 850 | B7-H1 (PD-1-ligand precursor) - Homo sapiens |
| NOV36a | CG56383-02 | 851 | 852 | Prickle-like 1 (Drosophila) - Mus musculus (Mouse), 879 aa |
| NOV36b | CG56383-01 | 853 | 854 | Prickle-like 1 (Drosophila) - Mus musculus (Mouse), 879 aa |
| NOV36c | SNP13382513 | 855 | 856 | Prickle-like 1 (Drosophila) - Mus musculus (Mouse), 879 aa |
| NOV37a | CG56449-04 | 857 | 858 | MEGF6 - Homo sapiens (Human), 1246 aa |
| NOV37b | CG56449-09 | 859 | 860 | MEGF6 - Homo sapiens (Human), 1246 aa |
| NOV37c | 191887507 | 861 | 862 | MEGF6 - Homo sapiens (Human), 1246 aa |
| NOV37d | 316351371 | 863 | 864 | MEGF6 - Homo sapiens (Human), 1246 aa |
| NOV37e | 316935396 | 865 | 866 | MEGF6 - Homo sapiens (Human), 1246 aa |
| NOV37f | 317004318 | 867 | 868 | MEGF6 - Homo sapiens (Human), 1246 aa |
| NOV37g | CG56449-01 | 869 | 870 | MEGF6 - Homo sapiens (Human), 1246 aa |
| NOV37h | CG56449-02 | 871 | 872 | MEGF6 - Homo sapiens (Human), 1246 aa |
| NOV37i | CG56449-03 | 873 | 874 | MEGF6 - Homo sapiens (Human), 1246 aa |
| NOV37j | CG56449-05 | 875 | 876 | MEGF6 - Homo sapiens (Human), 1246 aa |
| NOV37k | CG56449-06 | 877 | 878 | MEGF6 - Homo sapiens (Human), 1246 aa |
| NOV37l | CG56449-07 | 879 | 880 | MEGF6 - Homo sapiens (Human), 1246 aa |
| NOV37m | CG56449-08 | 881 | 882 | MEGF6 - Homo sapiens (Human), 1246 aa |
| NOV37n | CG56449-10 | 883 | 884 | MEGF6 - Homo sapiens (Human), 1246 aa |
| NOV37o | CG56449-11 | 885 | 886 | MEGF6 - Homo sapiens (Human), 1246 aa |
| NOV37p | SNP13382514 | 887 | 888 | MEGF6 - Homo sapiens (Human), 1246 aa |
| NOV38a | CG56594-01 | 889 | 890 | Claudin-19 - Homo sapiens |
| NOV38b | 277685552 | 891 | 892 | Claudin-19 - Homo sapiens |
| NOV38c | 277685649 | 893 | 894 | Claudin-19 - Homo sapiens |
| NOV38d | 277685616 | 895 | 896 | Claudin-19 - Homo sapiens |
| NOV39a | CG56653-08 | 897 | 898 | ficolin-1 precursor |
| NOV39b | 214374274 | 899 | 900 | ficolin-1 precursor |
| NOV39c | 214374256 | 901 | 902 | ficolin-1 precursor |
| NOV39d | 214374252 | 903 | 904 | ficolin-1 precursor |
| NOV39e | 214458492 | 905 | 906 | ficolin-1 precursor |
| NOV39f | 214458488 | 907 | 908 | ficolin-1 precursor |
| NOV39g | 214374236 | 909 | 910 | ficolin-1 precursor |
| NOV39h | CG56653-01 | 911 | 912 | ficolin-1 precursor |
| NOV39i | CG56653-02 | 913 | 914 | ficolin-1 precursor |
| NOV39j | CG56653-03 | 915 | 916 | ficolin-1 precursor |
| NOV39k | CG56653-04 | 917 | 918 | ficolin-1 precursor |
| NOV39l | CG56653-05 | 919 | 920 | ficolin-1 precursor |
| NOV39m | CG56653-06 | 921 | 922 | ficolin-1 precursor |
| NOV39n | CG56653-07 | 923 | 924 | ficolin-1 precursor |
| NOV39o | CG56653-09 | 925 | 926 | ficolin-1 precursor |
| NOV39p | CG56653-10 | 927 | 928 | ficolin-1 precursor |
| NOV39q | CG56653-11 | 929 | 930 | ficolin-1 precursor |
| NOV39r | CG56653-12 | 931 | 932 | ficolin-1 precursor |
| NOV40a | CG56806-01 | 933 | 934 | Heparan sulfate 6-O-sulfotransferase 3 - Homo sapiens |
| NOV40b | 248061366 | 935 | 936 | Heparan sulfate 6-O-sulfotransferase 3 - Homo sapiens |
| NOV40c | 246837961 | 937 | 938 | Heparan sulfate 6-O-sulfotransferase 3 - Homo sapiens |
| NOV40d | 246837965 | 939 | 940 | Heparan sulfate 6-O-sulfotransferase 3 - Homo sapiens |
| NOV40e | 246837975 | 941 | 942 | Heparan sulfate 6-O-sulfotransferase 3 - Homo sapiens |
| NOV40f | 248061376 | 943 | 944 | Heparan sulfate 6-O-sulfotransferase 3 - Homo sapiens |
| NOV40g | SNP13381685 | 945 | 946 | Heparan sulfate 6-O-sulfotransferase 3 - Homo sapiens |
| NOV40h | SNP13381686 | 947 | 948 | Heparan sulfate 6-O-sulfotransferase 3 - Homo sapiens |
| NOV41a | CG56878-01 | 949 | 950 | Protein OS-9 precursor - Homo sapiens |
| NOV41b | 175070399 | 951 | 952 | Protein OS-9 precursor - Homo sapiens |
| NOV41c | 175070432 | 953 | 954 | Protein OS-9 precursor - Homo sapiens |
| NOV41d | 175070419 | 955 | 956 | Protein OS-9 precursor - Homo sapiens |
| NOV41e | 175070438 | 957 | 958 | Protein OS-9 precursor - Homo sapiens |
| NOV41f | 175070408 | 959 | 960 | Protein OS-9 precursor - Homo sapiens |
| NOV41g | CG56878-02 | 961 | 962 | Protein OS-9 precursor - Homo sapiens |
| NOV41h | CG56878-03 | 963 | 964 | Protein OS-9 precursor - Homo sapiens |
| NOV41i | CG56878-04 | 965 | 966 | Protein OS-9 precursor - Homo sapiens |
| NOV41j | SNP13382511 | 967 | 968 | Protein OS-9 precursor - Homo sapiens |
| NOV41k | SNP13382506 | 969 | 970 | Protein OS-9 precursor - Homo sapiens |
| NOV42a | CG56904-01 | 971 | 972 | secreted protein LP255(a) |
| NOV42b | 272355087 | 973 | 974 | secreted protein LP255(a) |
| NOV42c | 246862506 | 975 | 976 | secreted protein LP255(a) |
| NOV42d | CG56904-04 | 977 | 978 | secreted protein LP255(a) |
| NOV42e | 268824694 | 979 | 980 | secreted protein LP255(a) |
| NOV42f | 283146542 | 981 | 982 | secreted protein LP255(a) |
| NOV42g | 308521214 | 983 | 984 | secreted protein LP255(a) |
| NOV42h | CG56904-02 | 985 | 986 | secreted protein LP255(a) |
| NOV42i | CG56904-03 | 987 | 988 | secreted protein LP255(a) |
| NOV42j | CG56904-05 | 989 | 990 | secreted protein LP255(a) |
| NOV42k | CG56904-06 | 991 | 992 | secreted protein LP255(a) |
| NOV42l | CG56904-07 | 993 | 994 | secreted protein LP255(a) |
| NOV42m | CG56904-08 | 995 | 996 | secreted protein LP255(a) |
| NOV43a | CG56914-01 | 997 | 998 | Hemicentin - Homo sapiens |
| NOV43b | 262802367 | 999 | 1000 | Hemicentin - Homo sapiens |
| NOV43c | CG56914-02 | 1001 | 1002 | Hemicentin - Homo sapiens |
| NOV43d | CG56914-03 | 1003 | 1004 | Hemicentin - Homo sapiens |
| NOV44a | CG56959-02 | 1005 | 1006 | Synaptotagmin 10 - Rattus norvegicus |
| NOV44b | CG56959-01 | 1007 | 1008 | Synaptotagmin 10 - Rattus norvegicus |
| NOV45a | CG57111-01 | 1009 | 1010 | Protocadherin 13 - Homo sapiens (Human), 947 aa |
| NOV45b | 277726328 | 1011 | 1012 | Protocadherin 13 - Homo sapiens (Human), 947 aa |
| NOV45c | CG57111-02 | 1013 | 1014 | Protocadherin 13 - Homo sapiens (Human), 947 aa |
| NOV45d | CG57111-03 | 1015 | 1016 | Protocadherin 13 - Homo sapiens (Human), 947 aa |
| NOV45e | CG57111-04 | 1017 | 1018 | Protocadherin 13 - Homo sapiens (Human), 947 aa |
| NOV46a | CG57409-05 | 1019 | 1020 | Glycosyl-phosphatidyl-inositol-MAM - Homo sapiens |
| NOV46b | 277731446 | 1021 | 1022 | Glycosyl-phosphatidyl-inositol-MAM - Homo sapiens |
| NOV46c | CG57409-07 | 1023 | 1024 | Glycosyl-phosphatidyl-inositol-MAM - Homo sapiens |
| NOV46d | 312102874 | 1025 | 1026 | Glycosyl-phosphatidyl-inositol-MAM - Homo sapiens |
| NOV46e | 312102899 | 1027 | 1028 | Glycosyl-phosphatidyl-inositol-MAM - Homo sapiens |
| NOV46f | CG57409-01 | 1029 | 1030 | Glycosyl-phosphatidyl-inositol-MAM - Homo sapiens |
| NOV46g | CG57409-02 | 1031 | 1032 | Glycosyl-phosphatidyl-inositol-MAM - Homo sapiens |
| NOV46h | CG57409-03 | 1033 | 1034 | Glycosyl-phosphatidyl-inositol-MAM - Homo sapiens |
| NOV46i | CG57409-04 | 1035 | 1036 | Glycosyl-phosphatidyl-inositol-MAM - Homo sapiens |
| NOV46j | CG57409-06 | 1037 | 1038 | Glycosyl-phosphatidyl-inositol-MAM - Homo sapiens |
| NOV46k | CG57409-08 | 1039 | 1040 | Glycosyl-phosphatidyl-inositol-MAM - Homo sapiens |
| NOV47a | CG57448-01 | 1041 | 1042 | Human cadherin-1 (CDHN-1) protein |
| NOV47b | 247846705 | 1043 | 1044 | Human cadherin-1 (CDHN-1) protein |
| NOV47c | 247846708 | 1045 | 1046 | Human cadherin-1 (CDHN-1) protein |
| NOV47d | 237580295 | 1047 | 1048 | Human cadherin-1 (CDHN-1) protein |
| NOV47e | 237579512 | 1049 | 1050 | Human cadherin-1 (CDHN-1) protein |
| NOV47f | CG57448-02 | 1051 | 1052 | Human cadherin-1 (CDHN-1) protein |
| NOV48a | CG57574-03 | 1053 | 1054 | Tectorin beta - Homo sapiens |
| NOV48b | CG57574-01 | 1055 | 1056 | Tectorin beta - Homo sapiens |
| NOV48c | CG57574-02 | 1057 | 1058 | Tectorin beta - Homo sapiens |
| NOV48d | CG57574-04 | 1059 | 1060 | Tectorin beta - Homo sapiens |
| NOV48e | CG57574-05 | 1061 | 1062 | Tectorin beta - Homo sapiens |
| NOV49a | CG57689-01 | 1063 | 1064 | Inferred: hemicentin - Mus musculus |
| NOV50a | CG58567-01 | 1065 | 1066 | Human cadherin (CAD) protein |
| NOV50b | 220087646 | 1067 | 1068 | Human cadherin (CAD) protein |
| NOV50c | 194877960 | 1069 | 1070 | Human cadherin (CAD) protein |
| NOV50d | 192589297 | 1071 | 1072 | Human cadherin (CAD) protein |
| NOV50e | CG58567-02 | 1073 | 1074 | Human cadherin (CAD) protein |
| NOV50f | CG58567-03 | 1075 | 1076 | Human cadherin (CAD) protein |
| NOV50g | CG58567-04 | 1077 | 1078 | Human cadherin (CAD) protein |
| NOV50h | CG58567-05 | 1079 | 1080 | Human cadherin (CAD) protein |
| NOV50i | CG58567-06 | 1081 | 1082 | Human cadherin (CAD) protein |
| NOV51a | CG59534-03 | 1083 | 1084 | Membrane glycoprotein LIG-1 - Homo sapiens |
| NOV51b | CG59534-01 | 1085 | 1086 | Membrane glycoprotein LIG-1 - Homo sapiens |
| NOV51c | CG59534-02 | 1087 | 1088 | Membrane glycoprotein LIG-1 - Homo sapiens |
| NOV51d | SNP13377255 | 1089 | 1090 | Membrane glycoprotein LIG-1 - Homo sapiens |
| NOV52a | CG59584-03 | 1091 | 1092 | Ovostatin precursor (Ovomacroglobulin) - Gallus gallus |
| NOV52b | CG59584-02 | 1093 | 1094 | Ovostatin precursor (Ovomacroglobulin) - Gallus gallus |
| NOV52c | 248210405 | 1095 | 1096 | Ovostatin precursor (Ovomacroglobulin) - Gallus gallus |
| NOV52d | 248210436 | 1097 | 1098 | Ovostatin precursor (Ovomacroglobulin) - Gallus gallus |
| NOV52e | 249357737 | 1099 | 1100 | Ovostatin precursor (Ovomacroglobulin) - Gallus gallus |
| NOV52f | 248210430 | 1101 | 1102 | Ovostatin precursor (Ovomacroglobulin) - Gallus gallus |
| NOV52g | 249357755 | 1103 | 1104 | Ovostatin precursor (Ovomacroglobulin) - Gallus gallus |
| NOV52h | CG59584-01 | 1105 | 1106 | Ovostatin precursor (Ovomacroglobulin) - Gallus gallus |
| NOV53a | CG56008-01 | 1107 | 1108 | |
| NOV54a | CG59905-01 | 1109 | 1110 | CUB and sushi multiple domains 1 protein - Homo sapiens |
| NOV54b | CG59905-03 | 1111 | 1112 | CUB and sushi multiple domains 1 protein - Homo sapiens |
| NOV54c | 275631102 | 1113 | 1114 | CUB and sushi multiple domains 1 protein - Homo sapiens |
| NOV54d | CG59905-02 | 1115 | 1116 | CUB and sushi multiple domains 1 protein - Homo sapiens |
| NOV54e | SNP13382527 | 1117 | 1118 | CUB and sushi multiple domains 1 protein - Homo sapiens |
| NOV54f | SNP13382520 | 1119 | 1120 | CUB and sushi multiple domains 1 protein - Homo sapiens |
| NOV55a | CG59932-01 | 1121 | 1122 | secreted protein sequence |
| NOV56a | CG92715-01 | 1123 | 1124 | secreted protein sequence |
| NOV56b | 248576233 | 1125 | 1126 | secreted protein sequence |
| NOV56c | CG92715-02 | 1127 | 1128 | secreted protein sequence |
| NOV56d | CG92715-03 | 1129 | 1130 | secreted protein sequence |
| NOV57a | CG92813-01 | 1131 | 1132 | Cadherin-related tumor suppressor precursor (Fat protein) - |
| | | | | Drosophila melanogaster |
| NOV57b | 260500971 | 1133 | 1134 | Cadherin-related tumor suppressor precursor (Fat protein) - |
| | | | | Drosophila melanogaster |
| NOV57c | 260500961 | 1135 | 1136 | Cadherin-related tumor suppressor precursor (Fat protein) - |
| | | | | Drosophila melanogaster |
| NOV57d | 258076331 | 1137 | 1138 | Cadherin-related tumor suppressor precursor (Fat protein) - |
| | | | | Drosophila melanogaster |
| NOV57e | 258076349 | 1139 | 1140 | Cadherin-related tumor suppressor precursor (Fat protein) - |
| | | | | Drosophila melanogaster |
| NOV57f | CG92813-02 | 1141 | 1142 | Cadherin-related tumor suppressor precursor (Fat protein) - |
| | | | | Drosophila melanogaster |
| NOV58a | CG93387-05 | 1143 | 1144 | insulin-responsive sequence DNA binding protein-1 - Homo sapiens |
| NOV58b | CG93387-01 | 1145 | 1146 | insulin-responsive sequence DNA winding protein-1 - Homo sapiens |
| NOV58c | CG93387-02 | 1147 | 1148 | insulin-responsive sequence DNA binding protein-1 - Homo sapiens |
| NOV58d | CG93387-03 | 1149 | 1150 | insulin-responsive sequence DNA winding protein-1 - Homo sapiens |
| NOV58e | CG93387-04 | 1151 | 1152 | insulin-responsive sequence DNA binding protein-1 - Homo sapiens |
| NOV58f | SNP13382519 | 1153 | 1154 | insulin-responsive sequence DNA winding protein-1 - Homo sapiens |
| NOV59a | CG93871-01 | 1155 | 1156 | insulin-responsive sequence DNA binding protein-1 - Homo sapiens |
| NOV59b | CG93871-05 | 1157 | 1158 | insulin-responsive sequence DNA winding protein-1 - Homo sapiens |
| NOV59c | 198488432 | 1159 | 1160 | insulin-responsive sequence DNA binding protein-1 - Homo sapiens |
| NOV59d | 198488424 | 1161 | 1162 | insulin-responsive sequence DNA winding protein-1 - Homo sapiens |
| NOV59e | 198488428 | 1163 | 1164 | insulin-responsive sequence DNA binding protein-1 - Homo sapiens |
| NOV59f | 198488440 | 1165 | 1166 | insulin-responsive sequence DNA winding protein-1 - Homo sapiens |
| NOV59g | CG93871-02 | 1167 | 1168 | insulin-responsive sequence DNA binding protein-1 - Homo sapiens |
| NOV59h | CG93871-03 | 1169 | 1170 | insulin-responsive sequence DNA winding protein-1 - Homo sapiens |
| NOV59i | CG93871-04 | 1171 | 1172 | insulin-responsive sequence DNA binding protein-1 - Homo sapiens |
| NOV60a | CG94946-01 | 1173 | 1174 | AGRIN precursor - Homo sapiens (Human), 2026 aa |
| NOV60b | 275631590 | 1175 | 1176 | AGRIN precursor - Homo sapiens (Human), 2026 aa |
| NOV60c | 275631564 | 1177 | 1178 | AGRIN precursor - Homo sapiens (Human), 2026 aa |
| NOV60d | CG94946-02 | 1179 | 1180 | AGRIN precursor - Homo sapiens (Human), 2026 aa |
| NOV60e | CG94946-03 | 1181 | 1182 | AGRIN precursor - Homo sapiens (Human), 2026 aa |
| NOV60f | CG94946-04 | 1183 | 1184 | AGRIN precursor - Homo sapiens (Human), 2026 aa |
| NOV60g | CG94946-05 | 1185 | 1186 | AGRIN precursor - Homo sapiens (Human), 2026 aa |
| NOV60h | CG94946-06 | 1187 | 1188 | AGRIN precursor - Homo sapiens (Human), 2026 aa |
| NOV60i | CG94946-07 | 1189 | 1190 | AGRIN precursor - Homo sapiens (Human), 2026 aa |
| NOV61a | CG96384-01 | 1191 | 1192 | colon cancer antigen protein |
| NOV61b | 277580745 | 1193 | 1194 | colon cancer antigen protein |
| NOV61c | CG96384-02 | 1195 | 1196 | colon cancer antigen protein |
| NOV61d | CG96384-03 | 1197 | 1198 | colon cancer antigen protein |
| NOV61e | CG96384-04 | 1199 | 1200 | colon cancer antigen protein |
| NOV62a | CG98011-01 | 1201 | 1202 | Neuronal transmembrane protein Slitrk4 - Mus musculus |
| NOV62b | 192586956 | 1203 | 1204 | Neuronal transmembrane protein Slitrk4 - Mus musculus |
| NOV62c | 191999007 | 1205 | 1206 | Neuronal transmembrane protein Slitrk4 - Mus musculus |
| NOV62d | CG98011-02 | 1207 | 1208 | Neuronal transmembrane protein Slitrk4 - Mus musculus |
| NOV62e | CG98011-03 | 1209 | 1210 | Neuronal transmembrane protein Slitrk4 - Mus musculus |
| NOV62f | CG98011-04 | 1211 | 1212 | Neuronal transmembrane protein Slitrk4 - Mus musculus |

Table A indicates the homology of NOVX polypeptides to known protein families. Thus, the nucleic acids and polypeptides, antibodies and related compounds according to the invention corresponding to a NOVX as identified in column 1 of Table A will be useful in therapeutic and diagnostic applications implicated in, for example, pathologies and disorders associated with the known protein families identified in column 5 of Table A.

Pathologies, diseases, disorders and condition and the like that are associated with NOVX sequences include, but are not limited to: e.g., cardiomyopathy, atherosclerosis, hypertension, congenital heart defects, aortic stenosis, atrial septal defect (ASD), vascular calcification, fibrosis, atrioventricular (A-V) canal defect, ductus arteriosus, pulmonary stenosis, subaortic stenosis, ventricular septal defect (VSD), valve diseases, tuberous sclerosis, scleroderma, obesity, metabolic disturbances associated with obesity, transplantation, osteoarthritis, rheumatoid arthritis, osteochondrodysplasia, adrenoleukodystrophy, congenital adrenal hyperplasia, prostate cancer, diabetes, metabolic disorders, neoplasm; adenocarcinoma, lymphoma, uterus cancer, fertility, glomerulonephritis, hemophilia, hypercoagulation, idiopathic thrombocytopenic purpura, immunodeficiencies, psoriasis, skin disorders, graft versus host disease, AIDS, bronchial asthma, lupus, Crohn's disease; inflammatory bowel disease, ulcerative colitis, multiple sclerosis, treatment of Albright Hereditary Ostoeodystrophy, infectious disease, anorexia, cancer-associated cachexia, cancer, neurodegenerative disorders, Alzheimer's Disease, Parkinson's Disorder, immune disorders, hematopoietic disorders, and the various dyslipidemias, schizophrenia, depression, asthma, emphysema, allergies, the metabolic syndrome X and wasting disorders associated with chronic diseases and various cancers, as well as conditions such as transplantation, neuroprotection, fertility, or regeneration (in vitro and in vivo).

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

Consistent with other known members of the family of proteins, identified in column 5 of Table A, the NOVX polypeptides of the present invention show homology to, and contain domains that are characteristic of, other members of such protein families. Details of the sequence relatedness and domain analysis for each NOVX are presented in Example A.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit diseases associated with the protein families listed in Table A.

The NOVX nucleic acids and polypeptides are also useful for detecting specific cell types. Details of the expression analysis for each NOVX are presented in Example C. Accordingly, the NOVX nucleic acids, polypeptides, antibodies and related compounds according to the invention will have diagnostic and therapeutic applications in the detection of a variety of diseases with differential expression in normal vs. diseased tissues, e.g. detection of a variety of cancers.

Additional utilities for NOVX nucleic acids and polypeptides according to the invention are disclosed herein.

NOVX Clones

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The NOVX genes and their corresponding encoded proteins are useful for preventing, treating or ameliorating medical conditions, e.g., by protein or gene therapy. Pathological conditions can be diagnosed by determining the amount of the new protein in a sample or by determining the presence of mutations in the new genes. Specific uses are described for each of the NOVX genes, based on the tissues in which they are most highly expressed. Uses include developing products for the diagnosis or treatment of a variety of diseases and disorders.

The NOVX nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) a biological defense weapon.

In one specific embodiment, the invention includes an isolated polypeptide comprising an amino acid sequence selected from the group consisting of: (a) a mature form of the amino acid sequence selected from the group consisting of SEQ ID NO: 2n, wherein n is an integer between 1 and 606; (b) a variant of a mature form of the amino acid sequence selected from the group consisting of SEQ ID NO: 2n, wherein n is an integer between 1 and 606, wherein any amino acid in the mature form is changed to a different amino acid, provided that no more than 15% of the amino acid residues in the sequence of the mature form are so changed; (c) an amino acid sequence selected from the group consisting of SEQ ID NO: 2n, wherein n is an integer between 1 and 606; (d) a variant of the amino acid sequence selected from the group consisting of SEQ ID NO:2n, wherein n is an integer between 1 and 606 wherein any amino acid specified in the chosen sequence is changed to a different amino acid,.provided that no more than 15% of the amino acid residues in the sequence are so changed; and (e) a fragment of any of (a) through (d).

In another specific embodiment, the invention includes an isolated nucleic acid molecule comprising a nucleic acid sequence encoding a polypeptide comprising an amino acid sequence selected from the group consisting of: (a) a mature form of the amino acid sequence given SEQ ID NO: 2n, wherein n is an integer between 1 and 606; (b) a variant of a mature form of the amino acid sequence selected from the group consisting of SEQ ID NO: 2n, wherein n is an integer between 1 and 606 wherein any amino acid in the mature form of the chosen sequence is changed to a different amino acid, provided that no more than 15% of the amino acid residues in the sequence of the mature form are so changed; (c) the amino acid sequence selected from the group consisting of SEQ ID NO: 2n, wherein n is an integer between 1 and 606; (d) a variant of the amino acid sequence selected from the group consisting of SEQ ID NO: 2n, wherein n is an integer between 1 and 606, in which any amino acid specified in the chosen sequence is changed to a different amino acid, provided that no more than 15% of the amino acid residues in the sequence are so changed; (e) a nucleic acid fragment encoding at least a portion of a polypeptide comprising the amino acid sequence selected from the group consisting of SEQ ID NO: 2n, wherein n is an integer between 1 and 606 or any variant of said polypeptide wherein any amino acid of the chosen sequence is changed to a different amino acid, provided that no more than 10% of the amino acid residues in the sequence are so changed; and (f) the complement of any of said nucleic acid molecules.

In yet another specific embodiment, the invention includes an isolated nucleic acid molecule, wherein said nucleic acid molecule comprises a nucleotide sequence selected from the group consisting of: (a) the nucleotide sequence selected from the group consisting of SEQ ID NO: 2n−1, wherein n is an integer between 1 and 606; (b) a nucleotide sequence wherein one or more nucleotides in the nucleotide sequence selected from the group consisting of SEQ ID NO: 2n−1, wherein n is an integer between 1 and 606 is changed from that selected from the group consisting of the chosen sequence to a different nucleotide provided that no more than 15% of the nucleotides are so changed; (c) a nucleic acid fragment of the sequence selected from the group consisting of SEQ ID NO: 2n−1, wherein n is an integer between 1 and 606; and (d) a nucleic acid fragment wherein one or more nucleotides in the nucleotide sequence selected from the group consisting of SEQ ID NO: 2n−1, wherein n is an integer between 1 and 606 is changed from that selected from the group consisting of the chosen sequence to a different nucleotide provided that no more than 15% of the nucleotides are so changed.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCk primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

A NOVX nucleic acid can encode a mature NOVX polypeptide. As used herein, a “mature” form of a polypeptide or protein disclosed in the present invention isthe product of a naturally occurring polypeptide or precursor form or proprotein. The naturally occurring polypeptide, precursor or proprotein includes, by way of nonlimiting example, the full-length gene product encoded by the corresponding gene. Alternatively, it may be defined as the polypeptide, precursor or proprotein encoded by an ORF described herein. The product “mature” form arises, by way of nonlimiting example, as a result of one or more naturally occurring processing steps that may take place within the cell (e.g., host cell) in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristylation or phosphorylation. In general, a mature polypeptide or protein may result from the operation of only one of these processes, or a combination of any of them.

The term “probe”, as utilized herein, refers to nucleic acid sequences of variable length, preferably between at least about 10 nucleotides (nt), about 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single-stranded or double-stranded and designed to have specificity in PCR, membrane-based hybridization technologies, or ELISA-like technologies.

The term “isolated” nucleic acid molecule, as used herein, is a nucleic acid that is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nuicleic acid is derived (e.g., brain, heart, liver, spleen, etc.). Moreover, an “isolated” nucleic acid molecule, such as a cDNA molecule, can be substantially free of other cellular material, or culture medium, or of chemical precursors or other chemicals.

A nucleic acid molecule of the invention, e.g., a nucleic acid molecule having the nucleotide sequence of SEQ ID NO:2n−1, wherein n is an integer between 1 and 606, or a complement of this nucleotide sequence, can be isolated using standard molecular biology techniques and the sequence information provided herein. Using all or a portion of the nucleic acid sequence of SEQ ID NO:2n−1, wherein n is an integer between 1 and 606, as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.) A nucleic acid of the invention can be amplified using cDNA, mRNA or alternatively, genomicDNA, as a template with appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NO:2n−1, wherein n is an integer between 1 and 606, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NO:2n−1, wherein n is an integer between 1 and 606, or a portion of this nucleotide sequence (e.g., a fragment that can be used as a probe or primer or a fragment encoding a biologically-active portion of a NOVX polypeptide). A nucleic acid molecule that is complementary to the nucleotide sequence of SEQ ID NO:2n−1, wherein n is an integer between 1 and 606, is one that is sufficiently complementary to the nucleotide sequence of SEQ ID NO:2n−1, wherein n is an integer between 1 and 606, that it can hydrogen bond with few or no mismatches to the nucleotide sequence shown in SEQ ID NO:2n−1, wherein n is an integer between 1 and 606, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

A “fragment” provided herein is defined as a sequence of at least 6 (contiguous) nucleic acids or at least 4 (contiguous) amino acids, a length sufficient to allow for specific hybridization in the case of nucleic acids or for specific recognition of an epitope in the case of amino acids, and is at most some portion less than a full length sequence. Fragments may be derived from any contiguous portion of a nucleic acid or amino acid sequence of choice.

A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3′ direction of the disclosed sequence.

A “derivative” is a nucleic acid sequence or amino acid sequence formed from the native compounds either directly, by modification or partial substitution. An “analog” is a nucleic acid sequence or amino acid sequence that has a structure similar to, but not identical to, the native compound, e.g. they differs from it in respect to certain components or side chains. Analogs may be synthetic or derived from a different evolutionary origin and may have a similar or opposite metabolic activity compared to wild type. A “homolog” is a nucleic acid sequence or amino acid sequence of a particular gene that is derived from different species.

Derivatives and analogs may be full length or other than full length. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins under stringent, moderately stringent, or low stringent conditions. See e.g. Ausubel, et al., CURENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences include those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for a NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NO:2n−1, wherein n is an integer between 1 and 606, as well as a polypeptide possessing NOVX biological activity. Various biological activities of the NOVX proteins are described below.

A NOVX polypeptide is encoded by the open reading frame (“ORF”) of a NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO:2n−1, wherein n is an integer between 1 and 606; or an anti-sense strand nucleotide sequence of SEQ ID NO:2n−1, wherein n is an integer between 1 and 606; or of a naturally occurring mutant of SEQ ID NO:2n−1, wherein n is an integer between 1 and 606.

Probes based on the human NOVX nucleotide sequences can be used to detect transcripts or genomic sequences encoding the same or homologous proteins. In various embodiments, the probe has a detectable label attached, e.g. the label can be a radioisotope, a fluorescent compound, an enzyme, or an enzyme co-factor. Such probes can be used as a part of a diagnostic test kit for identifying cells or tissues which mis-express a NOVX protein, such as by measuring a level of a NOVX-encoding nucleic acid in a sample of cells from a subject e.g., detecting NOVX mRNA levels or determining whether a genomic NOVX gene has been mutated or deleted. “A polypeptide having a biologically-active portion of a NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion of SEQ ID NO:2n−1, wherein n is an integer between 1 and 606, that encodes a polypeptide having a NOVX biological activity (the biological activities of the NOVX proteins are described below), expressing the encoded portion of NOVX protein (e.g., by recombinant expression in vitro) and assessing the activity of the encoded portion of NOVX.

NOVX Single Nucleotide Polymorphisms

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (Alderbom et al., Determination of Single Nucleotide Polymorphisms by Real-time Pyrophosphate DNA Sequencing. Genome Research. 10 (8) 1249-1265, 2000).

Variants are reported individually but any combination of all or a select subset of variants are also included as contemplated NOVX embodiments of the invention.

NOVX Nucleic Acid and Polypeptide Variants

The invention further encompasses nucleic acid molecules that differ from the nucleotide sequences of SEQ ID NO:2n−1, wherein n is an integer between 1 and 606, due to degeneracy of the genetic code and thus encode the same NOVX proteins as that encoded by the nucleotide sequences of SEQ ID NO:2n−1, wherein n is an integer between 1 and 606. In another embodiment, an isolated nucleic acid molecule of the invention has a nucleotide sequence encoding a protein having an amino acid sequence of SEQ ID NO:2n, wherein n is an integer between 1 and 606.

In addition to the human NOVX nucleotide sequences of SEQ ID NO:2n−1, wherein n is an integer between 1 and 606, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding a NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from a human SEQ ID NO:2n−1, wherein n is an integer between 1 and 606, are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:2n−1, wherein n is an integer between 1 and 606. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least about 65% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5 ° C lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to a sequence of SEQ ID NO:2n−1, wherein n is an integer between 1 and 606, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO:2n−1, wherein n is an integer between 1 and 606, or fragments, analogs or derivatives thereof, under conditions of moderate stringency is provided. A non-limiting example of moderate stringency hybridization conditions are hybridization in 6×SSC, 5×Reinhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel. et al. (eds.), 1993, CURRENT PROTOCOLS IN MOLECULAR BIOLOGY, John Wiley & Sons, NY, and Krieger, 1990; GENE TRANSFER AND EXPRESSION, A LABORATORY MANUAL, Stockton Press, N.Y.

In a third embodiment, a nucleic acid that is hybridizable to the nucleic acid molecule comprising the nucleotide sequences of SEQ ID NO:2n−1, wherein n is an integer between 1 and 606, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, N.Y.; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences of SEQ ID NO:2n−1, wherein n is an integer between 1 and 606, thereby leading to changes in the amino acid sequences of the encoded NOVX protein, without altering the functional ability of that NOVX protein. For example, nucleotide substitutions leading to amino acid substitutions at “non-essential” amino acid residues can be made in the sequence of SEQ ID NO:2n, wherein n is an integer between 1 and 606. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NO:2n−1, wherein n is an integer between 1 and 606, yet retain biological activity. In one embodiment, the isolated nucleic acid molecule comprises a nucleotide sequence encoding a protein, wherein the protein comprises an amino acid sequence at least about 40% homologous to the amino acid sequences of SEQ ID NO:2n, wherein n is an integer between 1 and 606. Preferably, the protein encoded by the nucleic acid molecule is at least about 60% homologous to SEQ ID NO:2n, wherein n is an integer between 1 and 606; more preferably at least about 70% homologous to SEQ ID NO:2n, wherein n is an integer between 1 and 606; still more preferably at least about 80% homologous to SEQ ID NO:2n, wherein n is an integer between 1 and 606; even more preferably at least about 90% homologous to SEQ ID NO:2n, wherein n is an integer between 1 and 606; and most preferably at least about 95% homologous to SEQ ID NO:2n, wherein n is an integer between 1 and 606.

An isolated nucleic acid molecule encoding a NOVX protein homologous to the protein of SEQ ID NO:2n, wherein n is an integer between 1 and 606, can be created by introducing one or more nucleotide substitutions, additions or deletions into the nucleotide sequence of SEQ ID NO:2n−1, wherein n is an integer between 1 and 606, such that one or more amino acid substitutions, additions or deletions are introduced into the encoded protein.

Mutations can be introduced any one of SEQ ID NO:2n−1, wherein n is an integer between 1 and 606, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of a NOVX coding sequence, such as by saturation mutagenesis, and the resultant mutants can be screened for NOVX biological activity to identify mutants that retain activity. Following mutagenesis of a nucleic acid of SEQ ID NO:2n−1, wherein n is an integer between 1 and 606, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and a NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Interfering RNA

In one aspect of the invention, NOVX gene expression can be attenuated by RNA interference. One approach well-known in the art is short interfering RNA (siRNA) mediated gene silencing where expression products of a NOVX gene are targeted by specific double stranded NOVX derived siRNA nucleotide sequences that are complementary to at least a 19-25 nt long segment of the NOVX gene transcript, including the 5′ untranslated (UT) region, the ORF, or the 3′ UT region. See, e.g., PCT applications WO00/44895, WO99/32619, WO01/75164, WO01/92513, WO 01/29058, WO01/89304, WO02/16620, and WO02/29858, each incorporated by reference herein in their entirety. Targeted genes can be a NOVX gene, or an upstream or downstream modulator of the NOVX gene. Nonlimiting examples of upstream or downstream modulators of a NOVX gene include, e.g., a transcription factor that binds the NOVX gene promoter, a kinase or phosphatase that interacts with a NOVX polypeptide, and polypeptides involved in a NOVX regulatory pathway.

According to the methods of the present invention, NOVX gene expression is silenced using short interfering RNA. A NOVX polynucleotide according to the invention includes a siRNA polynucleotide. Such a NOVX siRNA can be obtained using a NOVX polynucleotide sequence, for example, by processing the NOVX ribopolynucleotide sequence in a cell-free system, such as but not limited to a Drosophila extract, or by transcription of recombinant double stranded NOVX RNA or by chemical synthesis of nucleotide sequences homologous to a NOVX sequence. See, e.g., Tuschl, Zamore, Lehmann, Bartel and Sharp (1999), Genes & Dev. 13: 3191-3197, incorporated herein by reference in its entirety. When synthesized, a typical 0.2 micromolar-scale RNA synthesis provides about 1 milligram of siRNA, which is sufficient for 1000 transfection experiments using a 24-well tissue culture plate format.

The most efficient silencing is generally observed with siRNA duplexes composed of a 21-nt sense strand and a 21-nt antisense strand, paired in a manner to have a 2-nt 3′ overhang. The sequence of the 2-nt 3′ overhang makes an additional small contribution to the specificity of siRNA target recognition. The contribution to specificity is localized to the unpaired nucleotide adjacent to the first paired bases. In one embodiment, the nucleotides in the 3′ overhang are ribonucleotides. In an alternative embodiment, the nucleotides in the 3′ overhang are deoxyribonucleotides. Using 2′-deoxyribonucleotides in the 3′ overhangs is as efficient as using ribonucleotides, but deoxyribonucleotides are often cheaper to synthesize and are most likely more nuclease resistant.

A contemplated recombinant expression vector of the invention comprises a NOVX DNA molecule cloned into an expression vector comprising operatively-linked regulatory sequences flanking the NOVX sequence in a manner that allows for expression (by transcription of the DNA molecule) of both strands. An RNA molecule that is antisense to NOVX mRNA is transcribed by a first promoter (e.g., a promoter sequence 3′ of the cloned DNA) and an RNA molecule that is the sense strand for the NOVX mRNA is transcribed by a second promoter (e.g., a promoter sequence 5′ of the cloned DNA). The sense and antisense strands may hybridize in vivo to generate siRNA constructs for silencing of the NOVX gene. Alternatively, two constructs can be utilized to create the sense and anti-sense strands of a siRNA construct. Finally, cloned DNA can encode a construct having secondary structure, wherein a single transcript has both the sense and complementary antisense sequences from the target gene or genes. In an example of this embodiment, a hairpin RNAi product is homologous to all or a portion of the target gene. In another example, a hairpin RNAi product is a siRNA. The regulatory sequences flanking the NOVX sequence may be identical or may be different, such that their expression may be modulated independently, or in a temporal or spatial manner.

In a specific embodiment, siRNAs are transcribed intracellularly by cloning the NOVX gene templates into a vector containing, e.g., a RNA pol III transcription unit from the smaller nuclear RNA (snRNA) U6 or the human RNase P RNA H1. One example of a vector system is the GeneSuppressor™ RNA Interference kit (commercially available from Imgenex). The U6 and H1 promoters are members of the type III class of Pol III promoters. The +1 nucleotide of the U6-like promoters is always guanosine, whereas the +1 for H1 promoters is adenosine. The termination signal for these promoters is defined by five consecutive thymidines. The transcript is typically cleaved after the second uridine. Cleavage at this position generates a 3′ UU overhang in the expressed siRNA, which is similar to the 3′ overhangs of synthetic siRNAs. Any sequence less than 400 nucleotides in length can be transcribed by these promoter, therefore they are ideally suited for the expression of around 21-nucleotide siRNAs in, e.g., an approximately 50-nucleotide RNA stem-loop transcript.

A siRNA vector appears to have an advantage over synthetic siRNAs where long term knock-down of expression is desired. Cells transfected with a siRNA expression vector would experience steady, long-term mRNA inhibition. In contrast, cells transfected with exogenous synthetic siRNAs typically recover from mRNA suppression within seven days or ten rounds of cell division. The long-term gene silencing ability of siRNA expression vectors may provide for applications in gene therapy.

In general, siRNAs are chopped from longer dsRNA by an ATP-dependent ribonuclease called DICER. DICER is a member of the RNase III family of double-stranded RNA-specific endonucleases. The siRNAs assemble with cellular proteins into an endonuclease complex. In vitro studies in Drosophila suggest that the siRNAs/protein complex (siRNP) is then transferred to a second enzyme complex, called an RNA-induced silencing complex (RISC), which contains an endoribonuclease that is distinct from DICER. RISC uses the sequence encoded by the antisense siRNA strand to find and destroy mRNAs of complementary sequence. The siRNA thus acts as a guide, restricting the ribonuclease to cleave only mRNAs complementary to one of the two siRNA strands.

A NOVX mRNA region to be targeted by siRNA is generally selected from a desired NOVX sequence beginning 50 to 100 nt downstream of the start codon. Alternatively, 5′ or 3′ UTRs and regions nearby the start codon can be used but are generally avoided, as these may be richer in regulatory protein binding sites. UTR-binding proteins andlor translation initiation complexes may interfere with binding of the siRNP or RISC endonuclease complex. An initial BLAST homology search for the selected siRNA sequence is done against an available nucleotide sequence library to ensure that only one gene is targeted. Specificity of target recognition by siRNA duplexes indicate that a single point mutation located in the paired region of an siRNA duplex is sufficient to abolish target mRNA degradation. See, Elbashir et al. 2001 EMBO J. 20(23):6877-88. Hence, consideration should be taken to accommodate SNPs, polymorphisms, allelic variants or species-specific variations when targeting a desired gene.

In one embodiment, a complete NOVX siRNA experiment includes the proper negative control. A negative control siRNA generally has the same nucleotide composition as the NOVX siRNA but lack significant sequence homology to the genome. Typically, one would scramble the nucleotide sequence of the NOVX siRNA and do a homology search to make sure it lacks homology to any other gene.

Two independent NOVX siRNA duplexes can be used to knock-down a target NOVX gene. This helps to control for specificity of the silencing effect. In addition, expression of two independent genes can be simultaneously knocked down by using equal concentrations of different NOVX siRNA duplexes, e.g., a NOVX siRNA and an siRNA for a regulator of a NOVX gene or polypeptide. Availability of siRNA-associating proteins is believed to be more limiting than target mRNA accessibility.

A targeted NOVX region is typically a sequence of two adenines (AA) and two thymidines (TT) divided by a spacer region of nineteen (N19) residues (e.g., AA(N19)TT). A desirable spacer region has a G/C-content of approximately 30% to 70%, and more preferably of about 50%. If the sequence AA(N19)TT is not present in the target sequence, an alternative target region would be AA(N21). The sequence of the NOVX sense siRNA corresponds to (N19)TT or N21, respectively. In the latter case, conversion of the 3′ end of the sense siRNA to TT can be performed if such a sequence does not naturally occur in the NOVX polynucleotide. The rationale for this sequence conversion is to generate a symmetric duplex with respect to the sequence composition of the sense and antisense 3′ overhangs. Symmetric 3′ overhangs may help to ensure that the siRNPs are formed with approximately equal ratios of sense and antisense target RNA-cleaving siRNPs. See, e.g., Elbashir, Lendeckel and Tuschl (2001). Genes & Dev. 15: 188-200, incorporated by reference herein in its entirely. The modification of the overhang of the sense sequence of the siRNA duplex is not expected to affect targeted mRNA recognition, as the antisense siRNA strand guides target recognition.

Alternatively, if the NOVX target mRNA does not contain a suitable AA(N21) sequence, one may search for the sequence NA(N21). Further, the sequence of the sense strand and antisense strand may still be synthesized as 5′ (N19)TT, as it is believed that the sequence of the 3′-most nucleotide of the antisense siRNA does not contribute to specificity. Unlike antisense or ribozyme technology, the secondary structure of the target rnRNA does not appear to have a strong effect on silencing. See, Harborth, et al. (2001) J. Cell Science 114: 4557-4565, incorporated by reference in its entirety.

Transfection of NOVX siRNA duplexes can be achieved using standard nucleic acid transfection methods, for example, OLIGOFECTAMINE Reagent (commercially available from Invitrogen). An assay for NOVX gene silencing is generally performed approximately 2 days after transfection. No NOVX gene silencing has been observed in the absence of transfection reagent, allowing for a comparative analysis of the wild-type and silenced NOVX phenotypes. In a specific embodiment, for one well of a 24-well plate, approximately 0.84 μg of the siRNA duplex is generally sufficient. Cells are typically seeded the previous day, and are transfected at about 50% confluence. The choice of cell culture media and conditions are routine to those of skill in the art, and will vary with the choice of cell type. The efficiency of transfection may depend on the cell type, but also on the passage number and the confluency of the cells. The time and the manner of formation of siRNA-liposome complexes (e.g. inversion versus vortexing) are also critical. Low transfection efficiencies are the most frequent cause of unsuccessful NOVX silencing. The efficiency of transfection needs to be carefully examined for each new cell line to be used. Preferred cell are derived from a mammal, more preferably from a rodent such as a rat or mouse, and most preferably from a human. Where used for therapeutic treatment, the cells are preferentially autologous, although non-autologous cell sources are also contemplated as within the scope of the present invention.

For a control experiment, transfection of 0.84 μg single-stranded sense NOVX siRNA will have no effect on NOVX silencing, and 0.84 μg antisense siRNA has a weak silencing effect when compared to 0.84 μg of duplex siRNAs. Control experiments again allow for a comparative analysis of the wild-type and silenced NOVX phenotypes. To control for transfection efficiency, targeting of common proteins is typically performed, for example targeting of lamin A/C or transfection of a CMV-driven EGFP-expression plasmid (e.g. commercially available from Clontech). In the above example, a determination of the fraction of lamin A/C knockdown in cells is determined the next day by such techniques as immunofluorescence, Western blot, Northern blot or other similar assays for protein expression or gene expression. Lamin A/C monoclonal antibodies may be obtained from Santa Cruz Biotechnology.

Depending on the abundance and the half life (or turnover) of the targeted NOVX polynucleotide in a cell, a knock-down phenotype may become apparent after 1 to 3 days, or even later. In cases where no NOVX knock-down phenotype is observed, depletion of the NOVX polynucleotide may be observed by immunofluorescence or Western blotting. If the NOVX polynucleotide is still abundant after 3 days, cells need to be split and transferred to a fresh 24-well plate for re-transfection. If no knock-down of the targeted protein is observed, it may be desirable to analyze whether the target mRNA (NOVX or a NOVX upstream or downstream gene) was effectively destroyed by the transfected siRNA duplex. Two days after transfection, total RNA is prepared, reverse transcribed using a target-specific primer, and PCR-amplified with a primer pair covering at least one exon-exon junction in order to control for amplification of pre-mRNAs. RT/PCR of a non-targeted mRNA is also needed as control. Effective depletion of the mRNA yet undetectable reduction of target protein may indicate that a large reservoir of stable NOVX protein may exist in the cell. Multiple transfection in sufficiently long intervals may be necessary until the target protein is finally depleted to a point where a phenotype may become apparent. If multiple transfection steps are required, cells are split 2 to 3 days after transfection. The cells may be transfected immediately after splitting.

An inventive therapeutic method of the invention contemplates administering a NOVX siRNA construct as therapy to compensate for increased or aberrant NOVX expression or activity. The NOVX ribopolynucleotide is obtained and processed into siRNA fragments, or a NOVX siRNA is synthesized, as described above. The NOVX siRNA is administered to cells or tissues using known nucleic acid transfection techniques, as described above. A NOVX siRNA specific for a NOVX gene will decrease or knockdown NOVX transcription products, which will lead to reduced NOVX polypeptide production, resulting in reduced NOVX polypeptide activity in the cells or tissues.

The present invention also encompasses a method of treating a disease or condition associated with the presence of a NOVX protein in an individual comprising administering to the individual an RNAi construct that targets the mRNA of the protein (the mRNA that encodes the protein) for degradation. A specific RNAi construct includes a siRNA or a double stranded gene transcript that is processed into siRNAs. Upon treatment, the target protein is not produced or is not produced to the extent it would be in the absence of the treatment.

Where the NOVX gene function is not correlated with a known phenotype, a control sample of cells or tissues from healthy individuals provides a reference standard for determining NOVX expression levels. Expression levels are detected using the assays described, e.g., RT-PCR, Northern blotting, Western blotting, ELISA, and the like. A subject sample of cells or tissues is taken from a mammal, preferably a human subject, suffering from a disease state. The NOVX ribopolynucleotide is used to produce siRNA constructs, that are specific for the NOVX gene product. These cells or tissues are treated by administering NOVX siRNA's to the cells or tissues by methods described for the transfection of nucleic acids into a cell or tissue, and a change in NOVX polypeptide or polynucleotide expression is observed in the subject sample relative to the control sample, using the assays described. This NOVX gene knockdown approach provides a rapid method for determination of a NOVX minus (NOVX⁻) phenotype in the treated subject sample. The NOVX- phenotype observed in the treated subject sample thus serves as a marker for monitoring the course of a disease state during treatment.

In specific embodiments, a NOVX siRNA is used in therapy. Methods for the generation and use of a NOVX siRNA are known to those skilled in the art. Example techniques are provided below.

Production of RNAs

Sense RNA (ssRNA) and antisense RNA (asRNA) of NOVX are produced using known methods such as transcription in RNA expression vectors. In the initial experiments, the sense and antisense RNA are about 500 bases in length each. The produced ssRNA and asRNA (0.5 μM) in 10 mM Tris-HCl (pH 7.5) with 20 mM NaCl were heated to 95° C. for 1 min then cooled and annealed at room temperature for 12 to 16 h. The RNAs are precipitated and resuspended in lysis buffer (below). To monitor annealing, RNAs are electrophoresed in a 2% agarose gel in TBE buffer and stained with ethidium bromide. See, e.g., Sambrook et al., Molecular Cloning. Cold Spring Harbor Laboratory Press, Plainview, N.Y. (1989).

Lysate Preparation

Untreated rabbit reticulocyte lysate (Ambion) are assembled according to the manufacturer's directions. dsRNA is incubated in the lysate at 30° C. for 10 min prior to the addition of mRNAs. Then NOVX mRNAs are added and the incubation continued for an additional 60 min. The molar ratio of double stranded RNA and MrRNA is about 200:1. The NOVX mRNA is radiolabeled (using known techniques) and its stability is monitored by gel electrophoresis.

In a parallel experiment made with the same conditions, the double stranded RNA is internally radiolabeled with a ³²P-ATP. Reactions are stopped by the addition of 2× proteinase K buffer and deproteinized as described previously (Tuschl et al., Genes Dev., 13:3191-3197 (1999)). Products are analyzed by electrophoresis in 15% or 18% polyacrylamide sequencing gels using appropriateRNA standards. By monitoring the gels for radioactivity, the natural production of 10 to 25 nt RNAs from the double stranded RNA can be determined.

The band of double stranded RNA, about 21-23 bps, is eluded. The efficacy of these 21-23 mers for suppressing NOVX transcription is assayed in vitro using the same rabbit reticulocyte assay described above using 50 nanomolar of double stranded 21-23 mer for each assay. The sequence of these 21-23 mers is then determined using standard nucleic acid sequencing techniques.

RNA Preparation 21 nt RNAs, based on the sequence determined above, are chemically synthesized using Expedite RNA phosphoramidites and thymidine phosphoramidite (Proligo, Germany). Synthetic oligonucleotides are deprotected and gel-purified (Elbashir, Lendeckel, & Tuschl, Genes & Dev. 15, 188-200 (2001)), followed by Sep-Pak C18 cartridge (Waters, Milford, Mass., USA) purification (Tuschl, et al., Biochemistry, 32:11658-11668 (1993)).

These RNAs (20 μM) single strands are incubated in annealing buffer (100 mM potassium acetate, 30 mM HEPES-KOH at pH 7.4, 2 mM magnesium acetate) for 1 min at 90° C. followed by 1 h at 37° C.

Cell Culture

A cell culture known in the art to regularly express NOVX is propagated using standard conditions. 24 hours before transfection, at approx. 80% confluency, the cells are trypsinized and diluted 1:5 with fresh medium without antibiotics (1-3×105 cells/ml) and transferred to 24-well plates (500 ml/well). Transfection is performed using a commercially available lipofection kit and NOVX expression is monitored using standard techniques with positive and negative control. A positive control is cells that naturally express NOVX while a negative control is cells that do not express NOVX. Base-paired 21 and 22 nt siRNAs with overhanging 3′ ends mediate efficient sequence-specific mRNA degradation in lysates and in cell culture. Different concentrations of siRNAs are used. An efficient concentration for suppression in vitro in mamrnmalian culture is between 25 nM to 100 nM final concentration. This indicates that siRNAs are effective at concentrations that are several orders of magnitude below the concentrations applied in conventional antisense or ribozyme gene targeting experiments.

The above method provides a way both for the deduction of NOVX siRNA sequence and the use of such siRNA for in vitro suppression. In vivo suppression may be performed using the same siRNA using well known in vivo transfection or gene therapy transfection techniques.

Antisense Nucleic Acids

Another aspect of the invention pertains to isolated antisense nucleic acid molecules that are hybridizable to or complementary to the nucleic acid molecule comprising the nucleotide sequence of SEQ ID NO:2n−1, wherein n is an integer between 1 and 606, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of a NOVX protein of SEQ ID NO:2n, wherein n is an integer between 1 and 606, or antisense nucleic acids complementary to a NOVX nucleic acid sequence of SEQ ID NO:2n−1, wherein n is an integer between 1 and 606, are additionally provided.

In one embodiment, an antisense nucleic acid molecule is antisense to a “coding region” of the coding strand of a nucleotide sequence encoding a NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-carboxymethylaminomethyl-2-thiouridine, 5-(carboxyhydroxylmethyl) uracil, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 5-methoxyuracil, 3-methylcytosine, 5-methylcytosine, N6-adenine, 7-methylguanine, 5-methylaminomethyluracil, 5-methoxyaminomethyl-2-thiouracil, 2-thiouracil, 4-thiouracil, beta-D-mannosylqueosine, 5′-methoxycarboxymethyluracil, 2-methylthio-N6-isopentenyladenine, uracil-5-oxyacetic acid (v), wybutoxosine, pseudouracil, queosine, 2-thiocytosine, 5-methyl-2-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encodinga NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for a NOVX-encoding nucleic acid can be designed based upon the nucleotide sequence of a NOVX cDNA disclosed herein (i.e., SEQ ID NO:2n−1, wherein n is an integer between 1 and 606). For example, a derivative of a Tetrahymena L-19 IVS RNA can be constructed in which the nucleotide sequence of the active site is complementary to the nucleotide sequence to be cleaved in a NOVX-encoding mRNA. See, e.g., U.S. Pat. No. 4,987,07to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleotide bases are retained. The neutral backbone of PNAs has been shown to allow for specific hybridization to DNA and RNA under conditions of low ionic strength. The synthesis of PNA oligomer can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. nFor example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleotide bases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in any one of SEQ ID NO:2n, wherein n is an integer between 1 and 606. The invention also includes a mutant or variant protein any of whose residues may be changed from the corresponding residues shown in any one of SEQ ID NO:2n, wherein n is an integer between 1 and 606, while still encoding a protein that maintains its NOVX activities and physiological functions, or a functional fragment thereof.

In general, a NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, a NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other-chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence of SEQ ID NO:2n, wherein n is an integer between 1 and 606) that include fewer amino acids than the full-length NOVX proteins, and exhibit at least one activity of a NOVX protein. Typically, biologically-active portions comprise a domain or motif with at least one activity of the NOVX protein. A biologically-active portion of a NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence of SEQ ID NO:2n, wherein n is an integer between 1 and 606. In other embodiments, the NOVX protein is substantially homologous to SEQ ID NO:2n, wherein n is an integer between 1 and 606, and retains the functional activity of the protein of SEQ ID NO:2n, wherein n is an integer between 1 and 606, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence of SEQ ID NO:2n, wherein n is an integer between 1 and 606, and retains the functional activity of the NOVX proteins of SEQ ID NO:2n, wherein n is an integer between 1 and 606.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignmentwith a second amino or nucleic acid sequence). The amino acid residues or nucleotides at corresponding amino acid positions ornucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence of SEQ ID NO:2n−1, wherein n is an integer between 1 and 606.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G. U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, a NOVX “chimeric protein” or “fusion protein” comprises a NOVX polypeptide operatively-linked to a non-NOVX polypeptide. An “NOVX polypeptide” refers to a polypeptide having an amino acid sequence corresponding to a NOVX protein of SEQ ID NO:2n, wherein n is an integer between 1 and 606, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within a NOVX fusion protein the NOVX polypeptide can correspond to all or a portion of a NOVX protein. In one embodiment, a NOVX fusion protein comprises at least one biologically-active portion of a NOVX protein. In another embodiment, a NOVX fusion protein comprises at least two biologically-active portions of a NOVX protein. In yet another embodiment, a NOVX fusion protein comprises at least three biologically-active portions of a NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is a NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is a NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between a NOVX ligand and a NOVX protein on the surface of a cell, to thereby suppress NOVX-mediated signal transduction in vivo. The NOVX-immunoglobulin fusion proteins can be used to affect the bioavailability of a NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with a NOVX ligand.

A NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (ie., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of a NOVX protein. In one embodiment, a library of coding sequence fragments can be generated by treating a double stranded PCR fragment of a NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with SI nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the ! invention provides antigenic peptide fragments of the antigen for use as immunogens. An antigenic peptide fragment comprises at least 6 amino acid residues of the amino acid sequence of the full length protein, such as an amino acid sequence of SEQ ID NO:2n, wherein n is an integer between 1 and 606, and encompasses an epitope thereof such that X an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX that is located on the surface of the protein, e.g., a hydrophilic region. A hydrophobicity analysis of the human NOVX protein sequence will indicate which regions of a NOVX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

I The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. A NOVX polypeptide or a fragment thereof comprises at least one antigenic epitope. An anti-NOVX antibody of the present invention is said to specifically bind to antigen NOVX when the equilibrium binding constant (K_{D}) is ≦1 μM, preferably ≦100 nM, more preferably ≦10 nM, and most preferably ≦100 pM to about 1 pM, as measured by assays including radioligand binding assays or similar assays known to skilled artisans.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenbl, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells-or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Virginia. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding,1986). Suitable culture media for this purpose include, for example, Dulbecco's Modified Eagle's Medium and RPMI-1640 medium. Altematively,the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al.;,Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmnann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison ( Nature 368, 812-13 (1994)); Fishwild et al,( Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Mlstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamnine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Imunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulinr molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

Diagnostic Applications of Antibodies Directed Against the Proteins of the Invention

In one embodiment, methods for the screening of antibodies that possess the desired specificity include, but are not limited to, enzyme linked immunosorbent assay (ELISA) and other immunologically mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Antibodies directed against a NOVX protein of the invention may be used in methods known within the art relating to the localization and/or quantitation of a NOVX protein (e.g., for use in measuring levels of the NOVX protein within appropriate physiological samples, for use in diagnostic methods, for use in imaging the protein, and the like). In a given embodiment, antibodies specific to a NOVX protein, or derivative, fragment, analog or homolog thereof, that contain the antibody derived antigen binding domain, are utilized as pharmacologically active compounds (referred to hereinafter as “Therapeutics”).

An antibody specific for a NOVX protein of the invention (e.g., a monoclonal antibody or a polyclonal antibody) can be used to isolate a NOVX polypeptide by standard techniques, such as immunoaffinity, chromatography or immunoprecipitation. An antibody to a NOVX polypeptide can facilitate the purification of a natural NOVX antigen from cells, or of a recombinantly produced NOVX antigen expressed in host cells. Moreover, such an anti-NOVX antibody can be used to detect the antigenic NOVX protein (e.g., in a cellular lysate or cell supernatant) in order to evaluate the abundance and pattern of expression of the antigenic NOVX protein. Antibodies directed against a NOVX protein can be used diagnostically to monitor protein levels in tissue as part of a clinical testing procedure, e.g., to, for example, determine the efficacy of a given treatment regimen. Detection can be facilitated by coupling (ie., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical cormpositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

NOVX Recombinant Expression Vectors and Host Cells

Another aspect of the invention pertains to vectors, preferably expression vectors, containing a nucleic acid encoding a NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g.; in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and 17 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (In Vitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasrid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli , insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal. includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviralinfection) and allowing the oocyte to develop in a pseudopregnant female foster animal. The human NOVX cDNA sequences, i.e., any one of SEQ ID NO:2n−1, wherein n is an integer between 1 and 606, can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of a NOVX gene into which a deletion, addition or substitution has been introduced to thereby alter, e.g., functionally disrupt, the NOVX gene. The NOVX gene can be a human gene (e.g., the cDNA of any one of SEQ ID NO:2n−1, wherein n is an integer between 1 and 606), but more preferably, is a non-human homologue of a human NOVX gene. For example, a mouse homologue of human NOVX gene of SEQ ID NO:2n−1, wherein n is an integer between 1 and 606, can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through. the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in a NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of a NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are klown in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or atbiologically-active portion thereof, on the cell surface is contacted with a test compound and the ability of the test compound to bind to a NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with 1251, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with a NOVX target molecule. As used herein, a “target molecule” is a molecule with which a NOVX protein binds or interacts in nature, for example, a molecule on the surface of a cell which expresses a NOVX interacting protein, a molecule on the surface of a second cell, a molecule in the extracellular milieu, a molecule associated with the internal surface of a cell membrane or a cytoplasmic molecule. A NOVX target molecule can be a non-NOVX molecule or a NOVX protein or polypeptide of the invention. In one embodiment, a NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with a NOVX target molecule can be accomplished by one of the methods described above for determining direct binding. In one embodiment, determining the ability of the NOVX protein to bind to or interact with a NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to a NOVX target molecule by one of the methods described above for determining direct binding. In an alternative embodiment, determining the ability of the test compound to modulate the activity of NOVX protein can be accomplished by determining the ability of the NOVX protein further modulate a NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with a NOVX protein, wherein determining the ability of the test compound to interact with a NOVX protein comprises determining the ability of the NOVX protein to preferentially bind to or modulate the activity of a NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucaamide, decanoyl-N-methylglucarnide, Triton® X-100, Triton® X-114, Thesit®, Isotridecypoly(ethylene glycol ether)ₙ, N-dodecyl--N,N-dimethyl-3-ammonio-1-propane sulfonate, 3-(3-cholamidopropyl) dimethylamminiol-1-propane sulfonate (CHAPS), or 3-(3-cholamidopropyl)dimethylarmniniol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX rnRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences of SEQ ID NO:2n−1, wherein n is an integer between 1 and 606, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INERIANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLts).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If coding sequences, such as those of SEQ ID NO:2n−1, wherein n is an integer between 1 and 606, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in a NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.) Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX rnRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NO:2n−1, wherein n is an integer between 1 and 606, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with flucrescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genornic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for T determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genornic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in a NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a NOVX-protein, or the misexpression of the NOVX gene. For example, such genetic lesions can be detected by ascertaining the existence of at least one of: (i) a deletion of one or more nucleotides from a NOVX gene; (ii) an addition of one or more nucleotides to a NOVX gene; (iii) a substitution of one or more nucleotides of a NOVX gene, (iv) a chromosomal rearrangement of a NOVX gene; (v) an alteration in the level of a messenger RNA transcript of a NOVX gene, (vi) aberrant modification of a NOVX gene, such as of the methylation pattern of the genomic DNA, (vii) the presence of a non-wild-type splicing pattern of a messenger RNA transcript of a NOVX gene, (viii) a non-wild-type level of a NOVX protein, (ix) allelic loss of a NOVX gene, and (x) inappropriate post-translational modification of a NOVX protein. As described herein, there are a large number of assay techniques known in the art which can be used for detecting lesions in a NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with SI nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenonics

Agents, or modulators that have a stimulatory or inhibitory effect on NOVX activity (e.g., NOVX gene expression), as identified by a screening assay described herein can be administered to individuals to treat (prophylactically or therapeutically) disorders. The disorders include but are not limited to, e.g., those diseases, disorders and conditions listed above, and more particularly include those diseases, disorders, or conditions associated with homologs of a NOVX protein, such as those summarized in Table A.

In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome pregnancy zone protein precursor enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a NOVX protein, mRNA, or genomic DNA in the preadministration sample; (iii) obtaining one or more post-admninistration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include but are not limited to, e.g., those diseases, disorders and conditions listed above, and more particularly include those diseases, disorders, or conditions associated with homologs of a NOVX protein, such as those summarized in Table A.

These methods of treatment will be discussed more fully, below.

Diseases and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (ν) modulators ( i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, a NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of a NOVX protein, a peptide, a NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering a NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders. The disorders include but are not limited to, e.g., those diseases, disorders and conditions listed above, and more particularly include those diseases, disorders, or conditions associated with homologs of a NOVX protein, such as those summarized in Table A.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from diseases, disorders, conditions and the like, including but not limited to those listed herein.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example A

### Polynucleotide and Polypeptide Sequences, and Homology Data

### Example 1

The NOV1clone was analyzed, and the nucleotide and encoded polypeptide sequences are shown in Table 1A.

TABLE 1A
NOV1 Sequence Analysis

| | | |
|---|---|---|
| NOV1a, CG191083-01 | SEQ ID NO: 1 | 4494 bp |
| DNA Sequence | ORF Start: ATG at 59 | ORF Stop: TAA at 1394 |

CGAGAGCGGGAGTGAGCGAGCGAGCGAGTTGCCGAGCGCGCCCCGTCCCTCGCGCGCGATGCTCCCCT
GGACGGCGCTCGGCCTGGCCCTGAGCTTGCGGCTGGCGCTGGCGCGGAGCGGCGCAGAGCGCGGTCCA
CCAGCATCAGCCCCCCGAGGGGACCTGATGTTCCTGCTGGACAGCTCAGCCAGCGTCTCTCACTACGA
GTTCTCCCGGGTTCGGGAGTTTGTGGGGCAGCTGGTGGCTCCACTGCCCCTGGGCACCGGGGCCCTGC
GTGCCAGTCTGGTGCACGTGGGCAGTCGGCCATACACCGAGTTCCCCTTCGGCCAGCACAGCTCGGGT
GAGGCTGCCCAGGATGCGGTGCGTGCTTCTGCCCAGCGCATGGGTGACACCCACACTGGCCTGGCGCT
GGTCTATGCCAAGGAACAGCTGTTTGCTGAAGCATCAGGTGCCCGGCCAGGGGTGCCCAAAGTGCTGG
TGTGGGTGACAGATGGCGGCTCCAGCGACCCTGTGGGCCCCCCCATGCAGGAGCTCAAGGACCTGGGC
GTCACCGTGTTCATTGTCAGCACCGGCCGAGGCAACTTCCTGGAGCTGTCAGCCGCTGCCTCAGCCCC
TGCCGAGAAGCACCTGCACTTTGTGGACGTGGATGACCTGCACATCATTGTCCAAGAGCTGAGGGGCT
CCATTCTCGACGCGATGCGGCCGCAGCAGCTCCATGCCACGGAGATCACGTCCAGCGGCTTCCGCCTG
GCCTGGCCACCCCTGCTGACCGCAGACTCGGGCTACTATGTGCTGGAGCTGGTGCCCAGCGCCCAGCC
GGGGGCTGCAAGACGCCAGCAGCTGCCAGGGAACGCCACGGACTGGATCTGGGCCGGCCTCGACCCGG
ACACGGACTACGACGTGGCGCTAGTGCCTGAGTCCAACGTGCGCCTCCTGAGGCCCCAGATCCTGCGG
GTGCGCACGCGGCCCGGTGAGGCAGGGCCGGGGGCTTCGGGCCCGGAGTCGGGGGCTGGGCCGGCCCC
CACGCAGCTCGCCGCCCTCCCCGCCCCAGAGGAGGCCGGGCCAGAGCGCATCGTCATCTCCCACGCCC
GGCCGCGCAGCCTCCGCGTGAGTTGGGCCCCAGCGCTGGGCTCAGCCGCGGCGCTCGGCTACCACGTG
CAGTTCGGGCCGCTGCGGGGCGGGGAGGCGCAGCGGGTGGAGGTGCCCGCGGGCCGCAACTGCACCAC
GCTGCAGGGCCTGGCGCCGGGCACCGCCTACCTGGTGACCGTGACCGCCGCCTTCCGCTCGGGCCGCG
AGAGCGCGCTGTCCGCCAAGGCCTGCACGCCCGACGGCCCGCGCCCGCGCCCACGCCCCGTGCCCCGC
GCCCCGACCCCGGGGACCGCCAGCCGTGAGCCGTAAGCCGGCGTCCCCGCCCAGCCGAGAGGGCCGGC
GCCTACCTGAGGGCCCCTGTGTCCCGAACCCGGAGCGGAGGCGCCCAACCCGGCAGACGGGTGCAGGC
CCGGCCTTTCCCCACGCGGACTCCGCGCGACCCCGGCCCTCTCCCTGCGGCCGCAGGGCTTCCCCGCC
TGGCGCCTGCCCTCCAGGGCTGGGGCCTCGCCTGGCGGGACCCCGCAGCAGCCCCGGCCCCATCCCCC
CCCAGAGCCGGGCGTCGTGTGGGTCCGTGGGTGATAATTGAGAGCGTCAGACCCAGGACTGTTCAGGC
CAGCTTGGGTCCTGGACGTTGATAGGAAGCGGAAGGGGAATCGCGGGAAGCTGGCCCAGGTCAGGTCC
GCAAAGGCTTCTGAAGAAGAGGAAGGGCGAGTAGGGGCACCTGGACGCTGATGGTGGCCAGGATGCTC
AGCTGGCCAGGAGGGCAGCACCTGCTGGGGACGGTGGCCCTGCCTTCATGCCCAGGACACCAGCTGGG
TCCAGCTAGCAGCCACTGGGAATCAGAGGAATGGGGCAGAGCTGGCCATTCAGGACCTTGAGGACAGG
TGACCCCACCCGCCCACCGCCACTATCAGGCCCCGGGACCGCACTCACAGGAAACCTTCCGTCGTGAC
GGAGCACTTCCCAGGGGCCGCAGGGACGACACTCTCCAGGGAGGCCCCAGCAACCACACCATCTTCTT
GCTGTGAGAGGTCTCACCCCGGGCTACCTCCTGTCACTACTCACTGCCCTGGGGTCCGTGGGCAAGTT
GCCCAGGGTGGGGGTGCCTAGCCAGGTGCAGTCCCCGCCCCGCCTAGTCCTCGGCGTCACGCAATGCT
CACCTCGCCTCTTCCCCACTAACATCCCAGACTTTAAAATTCAGTAAATCAGATGTACACCGAGCCTC
CAGTCTCGCTAGGATGCTTTACTCCTCCACAAACAGCAGCCGCCGACAGGACAGCTTGTGAACGTCTC
CGCCTTTTGGGGTGTCCGGGGAGAGGAGGTGGAGCTGTCCCCTCCCAGTGTCAGGCCTCCTTGGTCCT
CATGTGCACCTTTTATTCAGAGAGCTTGGGCCCCCTTCCTTTGGAGCTGGGCCCAGGGCAGGTGGCTG
GAATGCAGAGGACAAGGGGGCTGGGGCCTCTGCAGGGCCATTCCCACCGCAGTCTCTGCCACGGCCCG
GAGCCCTGGGCCCAGGGCCACGCGGGCTCCCTCTGCTGGCAAAGCCACACCCTGCACCGCATGGGGTC
CACTGCCCCTTTCCCCACGTGGACTCTGCGTGACCCCAGGAACTGCAGCATTGAGGTGGTCTCAGTCC
CTCCCTCAGGTCTGGTGGGCATTGTGCCAGGGGCCACCTTCGGGACAGCTGGCCTTGGCACCGTCTCC
CAAGAACCCAGCTCTGTGCATCCCGTGGCCCCACCAGAAACCCAAGGCCACACCGCCCACCAGCTGGG
TGTGGTCACCATTCATGGAGGGCCGGCTGGGGCCAGGCTCACCACGCAGAGTTTGCCCTCCGGTGTGA
TGATGCATGAGACACAGTAGGCCCCCCTGCCTGTGTGTGCAGGGGGTGTCTGTGGGGGGGCCCTGCTG
GCTGGACTTTCGGTTTCTCCTGAGCTCCCCTTAGCCCCCAAGTCAGGCCACGGAGGCCTGAGTAGGAC
CCATGGGCTGTGTGTGTCACTGCAGGTGGCGTGCTCACAACTGCACTGCTGGTCGGCAGGTGGCCAGG
GTTGCAGGCCCGGGCCTCGGGCCCCACCTCCTCTGGAGGGGTAGGATCTCCTTCGTGCAGCCCCTGCC
CCCTACCCTGTTCTCCGGAGTGTTGGCAGATCTGAGCCCACGGTCACGTGAGAGGAGATGCCTCTTGC
ACACTGAGCCCAGGCCCAGCTCACGGAGCACCCTGCAGGTGGCAGCGGGGCACCAGGGGCTCATTCCT
GGTGGCCTCAGTGGCTTCTGTGGACGACTCAGACTCACATGAGAAGCTGGGAGGAGCTCCAGCTCTGC
AATCCCGAGAGGGCAGAGCGGGGGCCCTGGCCACAACCCTGCTCCCCACATACCCGTCTGGCAGAGGC
TCTGCCTCCCCTCTGCCCATCTCTGCACACCAAGGCTGAGCACAGCACAAGGCCTCACGGAGAGGGTG
AAAAGGCACTGCCGTCTCCATCTCAGTGCCAAGGAATGCCCCTTTCAGAAGCCCTCGTCTGAGAAGTG
CTGCCCCTTATGTAGCCCCAGGCATTTTAAAATCTTTGAATCTGCACCAAGTCTCAGAACAGCTCTGG
CCCCGGAGTGCTCCGTCCAGGCCGCTGTGAACCGGCTGTCCCCGCGTCTCCTCCACCTCCCCTGACAC
AATCCTGGCCCCGACTCAGTCCACCCAGGGTGCAGTGCAGAGGCTGATACCCGCCAGGACTTCCTTGC
CAAGGGCCCGGCCACGTCACAAGGCCACTGCACGCTTTTCGACATGCACCTGGAAATCGGGAAGGGCC
ATGCTGCAGTTGCTTCTAGTTGAGACGAGAAGAGCGACAAGGTTGTGATCCACGTGGCAGGTGTTCAG
AAGGCTGGGGGCGGGCAGCGCTGGGGAGAGCCCTGGGTACTTCGAGGAGACCCCGAAGGGAGGCTGCT
CCCACACCTGCGCCAGTTTCCACCCTCTCTGTGAGCAGGGCTGCGGTCACCTCCCACATCTGAAGAGA
ACCAACCTGAGGATTTCACGCTGGCTGCGTGCCAGACCAGTCCCTGACAGGTTGTGCGAGGCCCTTCG
CTGGACAGCCCATTGCTGGCCACTGGACGGAGAGGCAGAGGGGGCTGAAATTCGGGCCCATGCCTCTG
TGAGCGATGACGGAGCAACAGCTCTCCAGCACGTGAAGCTCTCCAGACAGCTGTTCGTGAGAAGCCAG
ACAGAGGCCTGGGGTCTCAGTCCAGATTTCTGGGGAGTGGGGTGTCCAAGCGTGGGCCACGCTGCTGG
GAGCCACCTAGGGAAGCAGGTCGCCTGTTTCTATAGTGACGGTAATAAACCAAGTCAATTTTCTATAA
AAAAAA

| | | | |
|---|---|---|---|
| NOV1a, CG191083-01 | SEQ ID NO: 2 | 445 aa | MW at 46803.5kD |
| Protein Sequence | | | |

MLPWTALGLALSLRLALARSGAERGPPASAPRGDLMFLLDSSASVSHYEFSRVREFVGQLVAPLPLGT
GALRASLVHVGSRPYTEFPFGQHSSGEAAQDAVRASAQRMGDTHTGLALVYAKEQLFAEASGARPGVP
KVLVWVTDGGSSDPVGPPMQELKDLGVTVFIVSTGRGNFLELSAAASAPAEKHLHFVDVDDLHIIVQE
LRGSILDAMRPQQLHATEITSSGFRLAWPPLLTADSGYYVLELVPSAQPGAARRQQLPGNATDWIWAG
LDPDTDYDVALVPESNVRLLRPQILRVRTRPGEAGPGASGPESGAGPAPTQLAALPAPEEAGPERIVI
HARPRSLRVSWAPALGSAAALGYHVQFGPLRGGEAQRVEVPAGPNCTTLQGLAPGTAYLVTVTAAFR
SGRESALSAKACTPDGPRPRPRPVPRAPTPGTASREP

Further analysis of the NOV1a protein yielded the following properties shown in Table 1B.

TABLE 1B
Protein Sequence Properties NOV1a

| | |
|---|---|
| SignalP analysis: | Cleavage site between residues 19 and 20 |
| PSORT II analysis: | |

| PSG: | a new signal peptide prediction method | |
|---|---|---|
| | N-region: | length 0; pos. chg 0; neg. chg 0 |
| | H-region: | length 13; peak value 8.36 |
| | PSG score: | 3.96 |

| | |
|---|---|
| GvH: | von Heijne's method for signal seq. recognition |
| | GvH score (threshold: −2.1): 2.40 |
| | possible cleavage site: between 18 and 19 |

>>> Seems to have a cleavable signal peptide (1 to 18)

| ALOM: | Klein et al's method for TM region allocation | |
|---|---|---|
| | Init position for calculation: 19 | |
| | Tentative number of TMS(s) for the threshold 0.5: 0 | |
| | number of TMS(s) . . . fixed | |
| | PERIPHERAL | Likelihood = 2.17 (at 60) |

| | |
|---|---|
| | ALOM score: 2.17 (number of TMSs: 0) |
| MTOP: | Prediction of membrane topology (Hartmann et al.) |
| | Center position for calculation: 9 |
| | Charge difference: 1.0 C(2.0)-N(1.0) |
| | C > N: C-terminal side will be inside |

>>>Caution: Inconsistent mtop result with signal peptide

| MITDISC: | discrimination of mitochondrial targeting seq | | | |
|---|---|---|---|---|
| | R content: | 2 | Hyd Moment(75): | 2.90 |
| | Hyd Moment(95): | 1.31 | G content: | 2 |
| | D/E content: | 1 | S/T content: | 3 |

| | |
|---|---|
| | Score: −4.22 |
| Gavel: | prediction of cleavage sites for mitochondrial preseq |
| | R-2 motif at 29 ARS\|GA |

NUCDISC: discrimination of nuclear localization signals

| | |
|---|---|
| | pat4: none |
| | pat7: none |
| | bipartite: none |
| | content of basic residues: 9.2% |
| | NLS Score: −0.47 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 76.7 |
| COIL: | Lupas's algorithm to detect coiled-coil regions |
| | total: 0 residues |

Final Results (k = 9/23):

33.3%: extracellular, including cell wall
33.3%: mitochondrial
22.2%: vacuolar
11.1%: endoplasmic reticulum

>> prediction for CG191083-01 is exc (k = 9)

A search of the NOV1a protein against the Geneseq database, a proprietary database that contains sequences published in patents and patent publication, yielded several homologous proteins shown in Table 1C.

TABLE 1C
Geneseq Results for NOV1a

| | | | Identities/ | |
|---|---|---|---|---|
| | | NOV1a | Similarities | |
| Geneseq | Protein/Organism/Length | Residues/ | for the | Expect |
| Identifier | [Patent #, Date] | Match Residues | Matched Region | Value |
| ABP69674 | Human polypeptide SEQ ID NO 1721 - | 1 . . . 445 | 445/445 (100%) | 0.0 |
| | Homo sapiens, 445 aa. | 1 . . . 445 | 445/445 (100%) | |
| | [WO200270539-A2, 12-SEP-2002] | | | |
| AAE32502 | Human Willebrand Factor A domain | 1 . . . 445 | 418/445 (93%) | 0.0 |
| | related-protein (WARP) - Homo | 1 . . . 418 | 418/445 (93%) | |
| | sapiens, 418 aa. [WO200288184-A1, | | | |
| | 07-NOV-2002] | | | |
| AAE32501 | Mouse Willebrand Factor A domain | 1 . . . 445 | 324/445 (72%) | 0.0 |
| | related-protein (WARP) - Mus sp, 415 | 1 . . . 415 | 356/445 (79%) | |
| | aa. [WO200288184-A1, 07-NOV-2002] | | | |
| AAW86326 | Kidney injury associated molecule | 1 . . . 445 | 325/445 (73%) | e−179 |
| | HW059 protein - Rattus sp, 421 aa. | 7 . . . 421 | 356/445 (79%) | |
| | [WO9853071-A1, 26-NOV-1998] | | | |
| AAB42581 | Human ORFX ORF2345 polypeptide | 3 . . . 296 | 274/294 (93%) | e−150 |
| | sequence SEQ ID NO: 4690 - Homo | 10 . . . 299 | 274/294 (93%) | |
| | sapiens, 299 aa. [WO200058473-A2, | | | |
| | 05-OCT-2000] | | | |

In a BLAST search of public sequence databases, the NOV1a protein was found to have homology to the proteins shown in the BLASTP data in Table 1D.

TABLE 1D
Public BLASTP Results for NOV1a

| | | | Identities/ | |
|---|---|---|---|---|
| Protein | | NOV1a | Similarities | |
| Accession | | Residues/ | for the | Expect |
| Number | Protein/Organism/Length | Match Residues | Matched Portion | Value |
| Q8R2Z5 | Hypothetical protein (VON | 1 . . . 445 | 325/445 (73%) | e−180 |
| | WILLEBRAND factor A-related | 1 . . . 415 | 356/445 (79%) | |
| | protein homolog) - Mus musculus | | | |
| | (Mouse), 415 aa. | | | |
| Q8C0Q7 | VON WILLEBRAND factor A-related | 1 . . . 445 | 324/445 (72%) | e−180 |
| | protein homolog - Mus musculus | 1 . . . 415 | 355/445 (78%) | |
| | (Mouse), 415 aa. | | | |
| Q923K3 | Von Willebrand factor A-related | 1 . . . 445 | 324/445 (72%) | e−180 |
| | protein - Mus musculus (Mouse), | 1 . . . 415 | 356/445 (79%) | |
| | 415 aa. | | | |
| CAC39705 | Sequence 47 from Patent EP1067182 - | 1 . . . 257 | 253/257 (98%) | e−142 |
| | Homo sapiens (Human), 285 aa. | 1 . . . 257 | 254/257 (98%) | |
| Q9H6J5 | Hypothetical protein FLJ22215 - | 213 . . . 445 | 233/233 (100%) | e−134 |
| | Homo sapiens (Human), 233 aa. | 1 . . . 233 | 233/233 (100%) | |

PFam analysis predicts that the NOV1a protein contains the domains shown in the Table 1E.

TABLE 1E
Domain Analysis of NOV1a

| | | Identities/ | | |
|---|---|---|---|---|
| | | Similarities | | |
| Pfam | | for the | | |
| Domain | NOV1a Match Region | Matched Region | | Expect Value |
| vwa | 34 . . . 205 | 68/194 | (35%) | 1.5e−40 |
| | | 132/194 | (68%) | |
| fn3 | 212 . . . 287 | 22/85 | (26%) | 0.18 |
| | | 52/85 | (61%) | |
| fn3 | 332 . . . 413 | 30/85 | (35%) | 1.4e−11 |
| | | 53/85 | (62%) | |

### Example 2

The NOV2 clone was analyzed, and the nucleotide and encoded polypeptide sequences are shown in Table 2A.

TABLE 2A
NOV2 Sequence Analysis

| | | |
|---|---|---|
| Nov2a, CG191745-01 | SEQ ID NO: 3 | 3462 bp |
| DNA Sequence | ORF Start: ATG at 2 | ORF Stop: TGA at 3443 |

GATGGGGCCAGAACGGACAGGGGCCGCGCCGCTGCCGCTGCTGCTGGTGTTAGCGCTCAGTCAAGGCA
TTTTAAATTGTTGTTTGGCCTACAATGTTGGTCTCCCAGAAGCAAAAATATTTTCCGGTCCTTCAAGT
GAACAGTTTGGCTATGCAGTGCAGCAGTTTATAAATCCAAAAGGCAACTGGTTACTGGTTGGTTCACC
CTGGAGTGGCTTTCCTGAGAACCGAATGGGAGATGTGTATAAATGTCCTGTTGACCTATCCACTGCCA
CATGTGAAAAACTAAATTTGCAAACTTCAACAAGCATTCCAAATGTTACTGAGATGAAAACCAACATG
AGCCTCGGCTTGATCCTCACCAGGAACATGGGAACTGGAGGTTTTCTCACATGTGGTCCTCTGTGGGC
ACAGCAATGTGGGAATCAGTATTACACAACGGGTGTGTGTTCTGACATCAGTCCTGATTTTCAGCTCT
CAGCCAGCTTCTCACCTGCAACTCAGCCCTGCCCTTCCCTCATAGATGTTGTGGTTGTGTGTGATGAA
TCAAATAGTATTTATCCTTGGGATGCAGTAAAGAATTTTTTGGAAAAATTTGTACAAGGCCTGGATAT
AGGCCCCACAAAGACACAGGTGGGGTTAATTCAGTATGCCAATAATCCAAGAGTTGTGTTTAACTTGA
ACACATATAAAACCAAAGAAGAAATGATTGTAGCAACATCCCAGACATCCCAATATGGTGGGGACCTC
ACAAACACATTCGGAGCAATTCAATATGCAAGAAAATATGCTTATTCAGCAGCTTCTGGTGGGCGACG
AAGTGCTACGAAAGTAATGGTAGTTGTAACTGACGGTGAATCACATGATGGTTCAATGTTGAAAGCTG
TGATTGATCAATGCAACCATGACAATATACTGAGGTTTGGCATAGCAGTTCTTGGGTACTTAAACAGA
AACGCCCTTGATACTAAAAATTTAATAAAAGAAATAAAAGCAATCGCTAGTATTCCAACAGAAAGATA
CTTTTTCAATGTGTCTGATGAAGCAGCTCTACTAGAAAAGGCTGGGACATTAGGAGAACAAATTTTCA
GCATTGAAGGTACTGTTCAAGGAGGAGACAACTTTCAGATGGAAATGTCACAAGTGGGATTCAGTGCA
GATTACTCTTCTCAAAATGATATTCTGATGCTGGGTGCAGTGGGAGCTTTTGGCTGGAGTGGGACCAT
TGTCCAGAAGACATCTCATGGCCATTTGATCTTTCCTAAACAAGCCTTTGACCAAATTCTGCAGGACA
GAAATCACAGTTCATATTTAGGTTACTCTGTGGCTGCAATTTCTACTGGAGAAAGCACTCACTTTGTT
GCTGGTGCTCCTCGGGCAAATTATACCGGCCAGATAGTGCTATATAGTGTGAATGAGAATGGCAATAT
CACGGTTATTCAGGCTCACCGAGGTGACCAGATTGGCTCCTATTTTGGTAGTGTGCTGTGTTCAGTTG
ATGTGGATAAAGACACCATTACAGACGTGCTCTTGGTAGGTGCACCAATGTACATGAGTGACCTAAAG
AAAGAGGAAGGAAGAGTCTACCTGTTTACTATCAAAGAGGGCATTTTGGGTCAGCACCAATTTCTTGA
AGGCCCCGAGGGCATTGAAAACACTCGATTTGGTTCAGCAATTGCAGCTCTTTCAGACATCAACATGG
ATGGCTTTAATGATGTGATTGTTGGTTCACCACTAGAAAATCAGAATTCTGGAGCTGTATACATTTAC
AATGGTCATCAGGGCACTATCCGCACAAAGTATTCCCAGAAAATCTTGGGATCCGATGGAGCCTTTAG
GAGCCATCTCCAGTACTTTGGGAGGTCCTTGGATGGCTATGGAGATTTAAATGGGGATTCCATCACCG
ATGTGTCTATTGGTGCCTTTGGACAAGTGGTTCAACTCTGGTCACAAAGTATTGCTGATGTAGCTATA
GAAGCTTCATTCACACCAGAAAAAATCACTTTGGTCAACAAGAATGCTCAGATAATTCTCAAACTCTG
CTTCAGTGCAAAGTTCAGACCTACTAAGCAAAACAATCAAGTGGCCATTGTATATAACATCACACTTG
ATGCAGATGGATTTTCATCCAGAGTAACCTCCAGGGGGTTATTTAAAGAAAACAATGAAAGGTGCCTG
CAGAAGAATATGGTAGTAAATCAAGCACAGAGTTGCCCCGAGCACATCATTTATATACAGGAGCCCTC
TGATGTTGTCAACTCTTTGGATTTGCGTGTGGACATCAGTCTGGAAAACCCTGGCACTAGCCCTGCCC
TTGAAGCCTATTCTGAGACTGCCAAGGTCTTCAGTATTCCTTTCCACAAAGACTGTGGTGAGGACGGA
CAGCAACCAAAACAAAAGGTTAACATTTTCAGTAACGCTGAAAAATAAAAGGGAAAGTGCATACAACA
GAAGTAACATGCCAGGTGGCTGCATCTCAGAAGTCTGTTGCCTGCGATGTAGGCTACCCTGCTTTAAA
GAGAGAACAACAGGTGACTTTTACTATTAACTTTGACTTCAATCTTCAAAACCTTCAGAATCAGGCGT
ATTCCTCTCCTGTATGATGCTGAAATTCACTTAACAAAGGTAACAACAGGAAGTGTTCCAGTAAGCAT
TGCAAACAGACAAGGCTGGTGACATCAGTTGTAATGCAGATATCAATCCACTGAAAATAGGACAAACA
TCTTCTTCTGTATCTTTCAAAAGTGAAAATTTCAGGCACACCAAAGAATTGAACTGCAGAACTGCTTC
CTGTAGTAATGTTACCTGCTGGTTGAAAGACGTTCACATGAAAGGAGAATACTTTGTTAATGTGACTA
CCAGAATTTGGAACGGGACTTTCGCATCATCAACGTTCCAGACAGTACAGCTAACGGCAGCTGCAGAA
ATCAACACCTATAACCCTGAGATATATGTGATTGAAGATAACACTGTTACGATTCCCCTGATGATAAT
GAAACCTGATGAGAAAGCCGAAGTACCAACAGGAGTTATAATAGGAAGTATAATTGCTGGAATCCTTT
TGCTGTTAGCTCTGGTTGCAATTTTATGGAAGCTCGGCTTCTTCAAAAGAAAATATGAAAAGATGACC
AAAAATCCAGATGAGATTGATGAGACCACAGAGCTCAGTAGCTGAACCAGCAGACCTACCTG

| | | | |
|---|---|---|---|
| NOV2a, CG191745-01 | SEQ ID NO: 4 | 1147 aa | MW at 125495.9kD |
| Protein Sequence | | | |

MGPERTGAAPLPLLLVLALSQGILNCCLAYNVGLPEAKIFSGPSSEQFGYAVQQFINPKGNWLLVGSP
WSGFPENRMGDVYKCPVDLSTATCEKLNLQTSTSIPNVTEMKTNMSLGLILTRNMGTGGFLTCGPLWA
QQCGNQYYTTGVCSDISPDFQLSASFSPATQPCPSLIDVVVVCDESNSIYPWDAVKNFLEKFVQGLDI
GPTKTQVGLIQYANNPRVVFNLNTYKTKEEMIVATSQTSQYGGDLTNTFGAIQYARKYAYSAASGGRR
SATKVMVVVIDGESHDGSMLKAVIDQCNHDNILRFGIAVLGYLNRNALDTKNLIKEIKAIASIPTERY
FFNVSDEAALLEKAGTLGEQIFSIEGTVQGGDNFQMEMSQVGFSADYSSQNDILMLGAVGAFGWSGTI
VQKTSHGHLIFPKQAFDQILQDRNHSSYLGYSVAAISTGESTHFVAGAPRANYTGQIVLYSVNENGNI
TVIQAHRGDQIGSYFGSVLCSVDVDKDTITDVLLVGAPMYMSDLKKEEGRVYLFTIKEGILGQHQFLE
GPEGIENTRFGSAIAALSDINMDGFNDVIVGSPLENQNSGAVYIYNGHQGTIRTKYSQKILGSDGAFR
SHLQYFGRSLDGYGDLNGDSITDVSIGAFGQVVQLWSQSIADVAIEASFTPEKITLVNKNAQIILKLC
FSAKFRPTKQNNQVAIVYNITLDADGFSSRVTSRGLFKENNERCLQKNMVVNQAQSCPEHIIYIQEPS
DVVNSLDLRVDISLENPGTSPALEAYSETAKVFSIPFHKDCGEDGLCISDLVLDVRQIPAAQEQPFIV
SNQNKRLTFSVTLKNKRESAYNTGIVVDFSENLFFASFSLPVDGTEVTCQVAASQKSVACDVGYPALK
REQQVTFTINFDFNLQNLQNQASLSFQALSESQEENKADNLVNLKIPLLYDAEIHLTKVTTGSVPVSM
ATVIIHIPQYTKEKNPLMYLTGVQTDKAGDISCNADINPLKIGQTSSSVSEKSENFRHTKELNCRTAS
CSNVTCWLKDVHMKGEYFVNVTTRIWNGTFASSTFQTVQLTAAAEINTYNPEIYVIEDNTVTIPLMIM
KPDEKAEVPTGVIIGSIIAGILLLLALVAILWKLGFFKRKYEKMTKNPDEIDETTELSS

Further analysis of the NOV2a protein yielded the following properties shown in Table 2B.

TABLE 2B
Protein Sequence Properties NOV2a

| | |
|---|---|
| SignalP analysis: | Cleavage site between residues 30 and 31 |
| PSORT II analysis: | |

| PSG: | a new signal peptide prediction method | |
|---|---|---|
| | N-region: | length 5; pos. chg 1; neg. chg 1 |
| | H-region: | length 30; peak value 9.82 |
| | PSG score: | 5.42 |

| | |
|---|---|
| GvH: | von Heijne's method for signal seq. recognition |
| | GvH score (threshold: −2.1): 1.12 |
| | possible cleavage site: between 22 and 23 |

>>> Seems to have a cleavable signal peptide (1 to 22)

| ALOM: | Klein et al's method for TM region allocation | | |
|---|---|---|---|
| | Init position for calculation: 23 | | |
| | Tentative number of TMS(s) for the threshold 0.5: 2 | | |
| | Number of TMS(s) for threshold 0.5: 1 | | |
| | INTEGRAL | Likelihood = | Transmembrane 1100-1116 |
| | | −13.27 | |

| | |
|---|---|
| PERIPHERAL | Likelihood = 0.95 (at 943) |

| | |
|---|---|
| | ALOM score: −13.27 (number of TMSs: 1) |
| MTOP: | Prediction of membrane topology (Hartmann et al.) |
| | Center position for calculation: 11 |
| | Charge difference: −2.0 C(−1.0)-N(1.0) |
| | N >= C: N-terminal side will be inside |

>>> membrane topology: type 1a (cytoplasmic tail 1117 to 1147)

| MITDISC: | discrimination of mitochondrial targeting seq | | | |
|---|---|---|---|---|
| | R content: | 1 | Hyd Moment(75): | 10.10 |
| | Hyd Moment(95): | 5.95 | G content: | 4 |
| | D/E content: | 2 | S/T content: | 2 |

| | |
|---|---|
| | Score: −6.90 |
| Gavel: | prediction of cleavage sites for mitochondrial preseq |
| | R-2 motif at 15 ERT\|GA |

NUCDISC: discrimination of nuclear localization signals

| | |
|---|---|
| | pat4: none |
| | pat7: none |
| | bipartite: none |
| | content of basic residues: 7.7% |
| | NLS Score: −0.47 |
| KDEL: | ER retention motif in the C-terminus: none |

ER Membrane Retention Signals:

XXRR-like motif in the N-terminus: GPER

none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: 1128
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 89 |
| COIL: | Lupas's algorithm to detect coiled-coil regions |
| | total: 0 residues |

Final Results (k = 9/23):

55.6%: endoplasmic reticulum
22.2%: Golgi
11.1%: plasma membrane
11.1%: extracellular, including cell wall

>> prediction for CG191745-01 is end (k = 9)

A search of the NOV2a protein against the Geneseq database, a proprietary database that contains sequences published in patents and patent publication, yielded several homologous proteins shown in Table 2C.

TABLE 2C
Geneseq Results for NOV2a

| | | NOV2a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAG79775 | Alpha2 integrin - Homo sapiens, 1181 | 1 . . . 1147 | 1146/1181 (97%) | 0.0 |
| | aa. [WO2002101070-A2, 19-DEC- | 1 . . . 1181 | 1147/1181 (97%) | |
| | 2002] | | | |
| ABU03548 | Angiogenesis-associated human | 1 . . . 1147 | 1146/1181 (97%) | 0.0 |
| | protein sequence #93 - Homo | 1 . . . 1181 | 1147/1181 (97%) | |
| | sapiens, 1181 aa. [WO200279492-A2, | | | |
| | 10-OCT-2002] | | | |
| ABU03617 | Human expressed protein tag (EPT) | 1 . . . 1147 | 1146/1181 (97%) | 0.0 |
| | #283 - Homo sapiens, 1181 aa. | 1 . . . 1181 | 1147/1181 (97%) | |
| | [WO200278524-A2, 10-OCT-2002] | | | |
| ABU03616 | Human expressed protein tag (EPT) | 1 . . . 1147 | 1146/1181 (97%) | 0.0 |
| | #282 - Homo sapiens, 1181 aa. | 1 . . . 1181 | 1147/1181 (97%) | |
| | [WO200278524-A2, 10-OCT-2002] | | | |
| ABU03614 | Human expressed protein tag (EPT) | 1 . . . 1147 | 1146/1181 (97%) | 0.0 |
| | #280 - Homo sapiens, 1181 aa. | 1 . . . 1181 | 1147/1181 (97%) | |
| | [WO200278524-A2, 10-OCT-2002] | | | |

In a BLAST search of public sequence databases, the NOV2a protein was found to have homology to the proteins shown in the BLASTP data in Table 2D.

TABLE 2D
Public BLASTP Results for NOV2a

| | | NOV2a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| P17301 | Integrin alpha-2 precursor (Platelet | 1 . . . 1147 | 1146/1181 | (97%) | 0.0 |
| | membrane glycoprotein la) (GPla) | 1 . . . 1181 | 1147/1181 | (97%) | |
| | (Collagen receptor) (VLA-2 alpha | | | | |
| | chain) (CD49b) - Homo sapiens | | | | |
| | (Human), 1181 aa. | | | | |
| P53710 | Integrin alpha-2 precursor (Platelet | 12 . . . 1147 | 986/1170 | (84%) | 0.0 |
| | membrane glycoprotein la) (GPla) | 1 . . . 1170 | 1069/1170 | (91%) | |
| | (Collagen receptor) (VLA-2 alpha | | | | |
| | chain) (CD49b) - Bos taurus (Bovine), | | | | |
| | 1170 aa (fragment). | | | | |
| Q62469 | Integrin alpha-2 precursor (Platelet | 1 . . . 1147 | 945/1181 | (80%) | 0.0 |
| | membrane glycoprotein la) (GPla) | 1 . . . 1178 | 1040/1181 | (88%) | |
| | (Collagen receptor) (VLA-2 alpha | | | | |
| | chain) (CD49b) - Mus musculus | | | | |
| | (Mouse), 1178 aa. | | | | |
| P18614 | Integrin alpha-1 precursor (Laminin | 1 . . . 1131 | 471/1202 | (39%) | 0.0 |
| | and collagen receptor) (VLA-1) | 1 . . . 1176 | 678/1202 | (56%) | |
| | (CD49a) - Rattus norvegicus (Rat), | | | | |
| | 1180 aa. | | | | |
| O42094 | ALPHA1 integrin - Gallus gallus | 29 . . . 1131 | 456/1179 | (38%) | 0.0 |
| | (Chicken), 1171 aa. | 17 . . . 1167 | 671/1179 | (56%) | |

PFam analysis predicts that the NOV2a protein contains the domains shown in the Table 2E.

TABLE 2E
Domain Analysis of NOV2a

| | | Identities/ | | |
|---|---|---|---|---|
| Pfam | NOV2a | Similarities for | | Expect |
| Domain | Match Region | the Matched Region | | Value |
| vwa | 174 . . . 361 | 64/203 | (32%) | 1.1e−47 |
| | | 146/203 | (72%) | |
| FG-GAP | 491 . . . 554 | 18/64 | (28%) | 0.00012 |
| | | 52/64 | (81%) | |
| FG-GAP | 555 . . . 602 | 14/52 | (27%) | 0.00062 |
| | | 37/52 | (71%) | |
| FG-GAP | 618 . . . 661 | 14/51 | (27%) | 0.073 |
| | | 32/51 | (63%) | |
| integrin_A | 1121 . . . 1135 | 8/15 | (53%) | 0.0048 |
| | | 14/15 | (93%) | |

### Example 3

The NOV3 clone was analyzed, and the nucleotide and encoded polypeptide sequences are shown in Table 3A.

TABLE 3A
NOV3 Sequence Analysis

| | | |
|---|---|---|
| NOV3a, CG50253-01 | SEQ ID NO: 5 | 1882 bp |
| DNA Sequence | ORF Start: ATG at 243 | ORF Stop: TAA at 1851 |

GGACACTGACATGGACTGAAGGAGTAGAAAAGAAGGGAGCGGGAGGGGGCTCCGGGCGCCGCGCAGCA
GACCTACTCCGGCCGCGCGCCTCGCCGCTGTCCTCCGGGAGCGGCAGCAGTAGCCCGGGCGGCGAGGG
CTGGGGGTTCCTCGAGACTCTCAGAGGGGCGCCTCCCATCGGCGCCCACCACCCCAACCTGTTCCTCG
CGCGCCACTGCGCTGCGCCCCAGGACCCGCTGCCCAACATGGATTTTCTCCTGGCGCTGGTGCTGGTA
TCCTCGCTCTACCTGCAGGCGGCCGCCGAGTTCGACGGGAGGTGGCCCAGGCAAATAGTGTCATCGAT
TGGCCTATGTCGTTATGGTGGGAGGATTGACTGCTGCTGGGGCTGGGCTCGCCAGTCTTGGGGACAGT
GTCAGCCTGTGTGCCAACCACGATGCAAACATGGTGAATGTATCGGGCCAAACAAGTGCAAGTGTCAT
CCTGGTTATGCTGGAAAAACCTGTAATCAAGATCTAAATGAGTGTGGCCTGAAGCCCCGGCCCTGTAA
GCACAGGTGCATGAACACTTACGGCAGCTACAAGTGCTACTGTCTCAACGGATATATGCTCATGCCGG
ATGGTTCCTGCTCAAGTGCCCTGACCTGCTCCATGGCAAACTGTCAGTATGGCTGTGATGTTGTTAAA
GGACAAATACGGTGCCAGTGCCCATCCCCTGGCCTGCAGCTGGCTCCTGATGGGAGGACCTGTGTAGA
TGTTGATGAATGTGCTACAGGAAGAGCCTCCTGCCCTAGATTTAGGCAATGTGTCAACACTTTTGGGA
GCTACATCTGCAAGTGTCATAAAGGCTTCGATCTCATGTATATTGGAGGCAAATATCAATGTCATGAC
ATAGACGAATGCTCACTTGGTCAGTATCAGTGCAGCAGCTTTGCTCGATGTTATAACATACGTGGGTC
CTACAAGTGCAAATGTAAAGAAGGATACCAGGGTGATGGACTGACTTGTGTGTATATCCCAAAAGTTA
TGATTGAACCTTCAGGTCCAATTCATGTACCAAAGGGAAATGGTACCATTTTAAAGGGTGACACAGGA
AATAATAATTGGATTCCTGATGTTGGAAGTACTTGGTGGCCTCCGAAGACACCATATATTCCTCCTAT
CATTACCAACAGGCCTACTTCTAAGCCAACAACAAGACCTACACCAAAGCCAACACCAATTCCTACTC
CACCACCACCACCACCCCTGCCAACAGAGCTCAGAACACCTCTACCACCTACAACCCCAGAAAGGCCA
ACCACCGGACTGACAACTATAGCACCAGCTGCCAGTACACCTCCAGGAGGGATTACAGTTGACAACAG
GGTACAGACAGACCCTCAGAAACCCAGAGGAGATGTGTTCAGTGTTCTGGTACACAGTTGTAATTTTG
ACCATGGACTTTGTGGATGGATCAGGGAGAAAGACAATGACTTGCACTGGGAACCAATCAGGGACCCA
GCAGGTGGACAATATCTGACAGTGTCGGCAGCCAAAGCCCCAGGGGGAAAAGCTGCACGCTTGGTGCT
ACCTCTCGGCCGCCTCATGCATTCAGGGGACCTGTGCCTGTCATTCAGGCACAAGGTGACGGGGCTGC
ACTCTGGCACACTCCAGGTGTTTGTGAGAAAACACGGTGCCCACGGAGCAGCCCTGTGGGGAAGAAAT
GGTGGCCATGGCTGGAGGCAAACACAGATCACCTTGCGAGGGGCTGACATCAAGAGCGTCGTCTTCAA
AGGTGAAAAAAGGCGTGGTCACACTGGGGAGATTGGATTAGATGATGTGAGCTTGAAAAAAGGCCACT
GCTCTGAAGAACGCTAACAACTCCAGAACTAACAATGAACTCCTAA

| | | | |
|---|---|---|---|
| NOV3a, CG50253-01 | SEQ ID NO: 6 | 536 aa | MW at 58650.8kD |
| Protein Sequence | | | |

| MDFLLALVLVSSLYLQAAAEFDGRWPRQIVSSIGLCRYGGRIDCCWGWARQSWGQCQPVCQPRCKHGE | | |
|---|---|---|
| CIGPNKCKCHPGYAGKTCNQDLNECGLKPRPCKHRCMNTYGSYKCYCLNGYMLMPDGSCSSALTCSMA | | |
| NCQYGCDVVKGQIRCQCPSPGLQLAPDGRTCVDVDECATGRASCPRFRQCVNTFGSYICKCHKGFDLM | | |
| YIGGKYQCHDIDECSLGQYQCSSFARCYNIRGSYKCKCKEGYQGDGLTCVYIPKVMIEPSGPIHVPKG | | |
| NGTILKGDTGNNNWIPDVGSTWWPPKTPYIPPIITNRPTSKPTTRPTPKPTPIPTPPPPPPLPTELRT | | |
| PLPPTTPERPTTGLTTIAPAASTPPGGITVDNRVQTDPQKPRGDVFSVLVHSCNFDHGLCGWIREKDN | | |
| DLHWEPIRDPAGGQYLTVSAAKAPGGKAARLVLPLGRLMHSGDLCLSFRHKVTGLHSGTLQVFVRKHG | | |
| AHGAALWGRNGGHGWRQTQITLRGADIKSVVFKGEKRRGHTGEIGLDDVSLKKGHCSEER | | |
| NOV3b, 250095765 | SEQ ID NO: 7 | 1765 bp |
| DNA Sequence | ORF Start: at 2 | ORF Stop: end of sequence |

| CACCGGATCCATGGATTTTCTCCTGGCGCTGGTGCTGGTATCCTCGCTCTACCTGCAGGCGGCCGCCG | | | | |
|---|---|---|---|---|
| AGTTCGACGGGAGGTGGCCCAGGCAAATAGTGTCATCGATTGGCCTATGTCGTTATGGTGGGAGGATT | | | | |
| AATAGCCAGGATAAGGTGCCAGCTCAAAGCTGTGTGCCAACCACGATGCAAACATGGTGAATGTATCG | | | | |
| GGCCAAACAAGTGCAAGTGTCATCCTGGTTATGCTGGAAAAACCTGTAATCAAGATCTAAATGAGTGT | | | | |
| GGCCTGAAGCCCCGGCCCTGTAAGCACAGGTGCATGAACACTTACGGCAGCTACAAGTGCTACTGTCT | | | | |
| CAACGGATATATGCTCATGCCGGATGGTTCCTGCTCAAGTGCCCTGACCTGCTCCATGGCAAACTGTC | | | | |
| AGTATGGCTGTGATGTTGTTAAAGGACAAATACGGTGCCAGTGCCCATCCCCTGGCCTGCAGCTGGCT | | | | |
| CCTGATGGGAGGACCTGTGTAGATGTTGATGAATGTGCTACAGGAAGAGCCTCCTGCCCTAGATTTAG | | | | |
| GCAATGTGTCAACACTTTTGGGAGCTACATCTGCAAGTGTCATAAAGGCTTCGATCTCATGTATATTG | | | | |
| GAGGCAAATATCAATGTCATGACATAGACGAATGCTCACTTGGTCAGTATCAGTGCAGCAGCTTTGCT | | | | |
| CGATGTTATAACGTACGTGGGTCCTACAAGTGCAAATGTAAAGAAGGATACCAGGGTGATGGACTGAC | | | | |
| TTGTGTGTATATCCCAAAAGTTATGATTGAACCTTCAGGTCCAATTCATGTACCAAAGGGAAATGGTA | | | | |
| CCATTTTAAAGGGTGACACAGGAAATAATAATTGGATTCCTGATGTTGGAAGTACTTGGTGGCCTCCG | | | | |
| AAGACACCATATATTCCTCCTATCATTACCAACAGGCCTACTTCTAAGCCAACAACAAGACCTACACC | | | | |
| AAAGCCAACACCAATTCCTACTCCACCACCACCACCACCCCTGCCAACAGAGCTCAGAACACCTCTAC | | | | |
| CACCTACAACCCCAGAAAGGCCAACCACCGGACTGACAACTATAGCACCAGCTGCCAGTACACCTCCA | | | | |
| GGAGGGATTACAGTTGACAACAGGGTACAGACAGACCCTCAGAAACCCAGAGGAGATGTGTTCATTCC | | | | |
| ACGGCAACCTTCAAATGACTTGTTTGAAATATTTGAAATAGAAAGAGGAGTCAGTGCAGACGATGAAG | | | | |
| CAAAGGATGATCCAGGTGTTCTGGTACACAGTTGTAATTTTGACCATGGACTTTGTGGATGGATCAGG | | | | |
| GAGAAAGACAATGACTTGCACTGGGAACCAATCAGGGACCCAGCAGGTGGACAATATCTGACAGTGTC | | | | |
| GGCAGCCAAAGCCCCAGGGGGAAAAGCTGCACGCTTGGTGCTACCTCTCGGCCGCCTCATGCATTCAG | | | | |
| GGGACCTGTGCCTGTCATTCAGGCACAAGGTGACGGGGCTGCACTCTGGCACACTCCAGGTGTTTGTG | | | | |
| AGAAAACACGGTGCCCACGGAGCAGCCCTGTGGGGAAGAAATGGTGGCCATGGCTGGAGGCAAACACA | | | | |
| GATCACCTTGCGAGGGGCTGACATCAAGAGCGTCGTCTTCAAAGGTGAAAAAAGGCGTGGTCACACTG | | | | |
| GGGAGATTGGATTAGATGATGTGAGCTTGAAAAAAGGCCACTGCTCTGAAGAACGCGTCGACGGC | | | | |
| | NOV3b, 250095765 | SEQ ID NO: 8 | 1588 aa | MW at 64525.3kD |
| | Protein Sequence | | | |

| TGSMDFLLALVLVSSLYLQAAAEFDGRWPRQIVSSIGLCRYGGRIDCCWGWARQSWGQCQPFYVLRQR | | |
|---|---|---|
| IARIRCQLKAVCQPRCKHGECIGPNKCKCHPGYAGKTCNQDLNECGLKPRPCKHRCMNTYGSYKCYCL | | |
| NGYMLMPDGSCSSALTCSMANCQYGCDVVKGQIRCQCPSPGLQLAPDGRTCVDVDECATGRASCPRFR | | |
| QCVNTFGSYICKCHKGFDLMYIGGKYQCHDIDECSLGQYQCSSFARCYNVRGSYKCKCKEGYQGDGLT | | |
| CVYIPKVMIEPSGPIHVPKGNGTILKGDTGNNNWIPDVGSTWWPPKTPYIPPIITNRPTSKPTTRPTP | | |
| KPTPIPTPPPPPPLPTELRTPLPPTTPERPTTGLTTIAPAASTPPGGITVDNRVQTDPQKPRGDVFIP | | |
| RQPSNDLFEIFEIERGVSADDEAKDDPGVLVHSCNFDHGLCGWIREKDNDLHWEPIRDPAGGQYLTVS | | |
| AAKAPGGKAARLVLPLGRLMHSGDLCLSFRHKVTGLHSGTLQVFVRKHGAHGAALWGRNGGHGWRQTQ | | |
| ITLRGADIKSVVFKGEKRRGHTGEIGLDDVSLKKGHCSEERVDG | | |
| NOV3c, 250095742 | SEQ ID NO: 9 | 1627 bp |
| DNA Sequence | ORF Start: at 2 | ORF Stop: end of sequence |

| CACCGGATCCATGGATTTTCTCCTGGCGCTGGTGCTGGTATCCTCGCTCTACCTGCAGGCGGCCGCCG | | | | |
|---|---|---|---|---|
| AGTTCGACGGGAGGTGGCCCAGGCAAATAGTGTCATCGATTGGCCTATGTCGTTATGGTGGGAGGATT | | | | |
| GACTGCTGCTGGGGCTGGGCTCGCCAGTCTTGGGGACAGTGTCAGCCTGTGTGCCAACCACGATGCAA | | | | |
| ACATGGTGAATGTATCGGGCCAAACAAGTGCAAGTGTCATCCTGGTTATGCTGGAAAAACCTGTAATC | | | | |
| AAGATCTAAATGAGTGTGGCCTGAAGCCCCGGCCCTGTAAGCACAGGTGCATGAACACTTACGGCAGC | | | | |
| TACAAGTGCTACTGTCTCAACGGATATATGCTCATGCCGGATGGTTCCTGCTCAAGTGCCCTGACCTG | | | | |
| CTCCATGGCAAACTGTCAGTATGGCTGTGATGTTGTTAAAGGACAAATACGGTGCCAGTGCCCATCCC | | | | |
| CTGGCCTGCACCTGGCTCCTGATGGGAGGACCTGTGTAGATGTTGATGAATGTGCTACAGGAAGAGCC | | | | |
| TCCTGCCCTAGATTTAGGCAATGTGTCAACACTTTTGGGAGCTACATCTGCAAGTGTCATAAAGGCTT | | | | |
| CGATCTCATGTATATTGGAGGCAAATATCAATGTCATGACATAGACGAATGCTCACTTGGTCAGTATC | | | | |
| AGTGCAGCAGCTTTGCTCGATGTTATAACGTACGTGGGTCCTACAAGTGCAAATGTAAAGAAGGATAC | | | | |
| CAGGGTGATGGACTGACTTGTGTGTATATCCCAAAAGTTATGATTGAACCTTCAGGTCCAATTCATGT | | | | |
| ACCAAAGGGAAATGGTACCATTTTAAAGGGTGACACAGGAAATAATAATTGGATTCCTGATGTTGGAA | | | | |
| GTACTTGGTGGCCTCCGAAGACACCATATATTCCTCCTATCATTACCAACAGGCCTACTTCTAAGCCA | | | | |
| ACAACAAGACCTACACCAAAGCCAACACCAATTCCTACTCCACCACCACCACCACCCCTGCCAACAGA | | | | |
| GCTCAGAACACCTCTACCACCTACAACCCCAGAAAGGCCAACCACCGGACTGACAACTATAGCACCAG | | | | |
| CTGCCAGTACACCTCCAGGAGGGATTACAGTTGACAACAGGGTACAGACAGACCCTCAGAAACCCAGA | | | | |
| GGAGATGTGTTCAGTGTTCTGGTACACAGTTGTAATTTTGACCATGGACTTTGTGGATGGATCAGGGA | | | | |
| GAAAGACAATGACTTGCACTGGGAACCAATCAGGGACCCAGCAGGTGGACAATATCTGACAGTGTCGG | | | | |
| CAGCCAAAGCCCCAGGGGGAAAAGCTGCACGCTTGGTGCTACCTCTCGGCCGCCTCATGCATTCAGGG | | | | |
| GACCTGTGCCTGTCATTCAGGCACAAGGTGACGGGGCTGCACTCTGGCACACTCCAGGTGTTTGTGAG | | | | |
| AAAACACGGTGCCCACGGAGCAGCCCTGTGGGGAAGAAATGGTGGCCATGGCTGGAGGCAAACACAGA | | | | |
| TCACCTTGCGAGGGGCTGACATCAAGAGCGTCGTCTTCAAAGGTGAAAAAAGGCGTGGTCACACTGGG | | | | |
| GAGATTGGATTAGATGATGTGAGCTTGAAAAAAGGCCACTGCTCTGAAGAACGCGTCGACGGC | | | | |
| | NOV3c, 250095742 | SEQ ID NO: 10 | 542 aa | MW at 59162.3kD |
| | Protein Sequence | | | |

| TGSMDFLLALVLVSSLYLQAAAEFDGRWPRQIVSSIGLCRYGGRIDCCWGWARQSWGQCQPVCQPRCK | | |
|---|---|---|
| HGECIGPNKCKCHPGYAGKTCNQDLNECGLKPRPCKHRCMNTYGSYKCYCLNGYMLMPDGSCSSALTC | | |
| SMANCQYGCDVVKGQIRCQCPSPGLHIAPDGRTCVDVDECATGRASCPRFRQCVNTFGSYTCKCHKGF | | |
| DLMYIGGKYQCHDIDECSLGQYQCSSFARCYNVRGSYKCKCKEGYQGDGLTCVYIPKVMIEPSGPIHV | | |
| PKGNGTILKGDTGNNNWIPDVGSTWWPPKTPYIPPIITNRPTSKPTTRPTPKPTPIPTPPPPPPLPTE | | |
| LRTPLPPTTPERPTTGLTTIAPAASTPPGGITVDNRVQTDPQKPRGDVFSVLVHSCNFDHGLCGWIRE | | |
| KDNDLHWEPIRDPAGGQYLTVSAAKAPGGKAARLVLPLGRLMHSGDLCLSFRHKVTGLHSGTLQVFVR | | |
| KHGAHGAALWGRNGGHGWRQTQITLRGADIKSVVFKGEKRRGHTGEIGLDDVSLKKGHCSEERVDG | | |
| Nov3d, 250095779 | SEQ ID NO: 11 | 1765 bp |
| DNA Sequence | ORF Start: at 2 | ORF Stop: end of sequence |

| CACCGGATCCATGGATTTTCTCCTGGCGCTGGTGCTGGTATCCTCGCTCTACCTGCAGGCGGCCGCCG | | | | |
|---|---|---|---|---|
| AGTTCGACGGGAGGTGGCCCAGGCAAATAGTGTCATCGATTGGCCTATGTCGTTATGGTGGGAGGATT | | | | |
| GACTGCTGCTGGGGCTGGGCTCGCCAGTCTTGGGGACAGTGTCAGCCTTTCTACGTCTTAAGGCAGAG | | | | |
| AATAGCCAGGATAAGGTGCCAGCTCAAAGCTGTGTGCCAACCACGATGCAAACATGGTGAATGTATCG | | | | |
| GGCCAAACAAGTGCAAGTGTCATCCTGGTTATGCTGGAAAAACCTGTAATCAAGATCTAAATGAGTGT | | | | |
| CAACGGATATATGCTCATGCCGGATGGTTCCTGCTCAAGTGCCCTGACCTGCTCCATGGCAAACTGTC | | | | |
| AGTATGGCTGTGATGTTGTTAAAGGACAAATACGGTGCCAGTGCCCATCCCCTGGCCTGCACCTGGCT | | | | |
| CCTGATGGGAGGACCTGTGTAGATGTTGATGAATGTGCTACAGGAAGAGCCTCCTGCCCTAGATTTAG | | | | |
| GCAATGTGTCAACACTTTTGGGAGCTACATCTGCAAGTGTCATAAAGGCTTCGATCTCATGTATATTG | | | | |
| GAGGCAAATATCAATGTCATGACATAGACGAATGCTCACTTGGTCAGTATCAGTGCAGCAGCTTTGCT | | | | |
| CGATGTTATAACGTACGTGGGTCCTACAAGTGCAAATGTAAAGAAGGATACCAGGGTGATGGACTGAC | | | | |
| TTGTGTGTATATCCCAAAAGTTATGATTGAACCTTCAGGTCCAATTCATGTACCAAAGGGAAATGGTA | | | | |
| CCATTTTAAAGGGTGACACAGGAAATAATAATTGGATTCCTGATGTTGGAAGTACTTGGTGGCCTCCG | | | | |
| AAGACACCATATATTCCTCCTATCATTACCAACAGGCCTACTTCTAAGCCAACAACAAGACCTACACC | | | | |
| AAAGCCAACACCAATTCCTACTCCACCACCACCACCACCCCTGCCAACAGAGCTCAGAACACCTCTAC | | | | |
| CACCTACAACCCCAGAAAGGCCAACCACCGGACTGACAACTATAGCACCAGCTGCCAGTACACCTCCA | | | | |
| GGAGGGATTACAGTTGACAACAGGGTACAGACAGACCCTCAGAAACCCAGAGGAGATGTGTTCATTCC | | | | |
| ACGGCAACCTTCAAATGACTTGTTTGAAATATTTGAAATAGAAAGAGGAGTCAGTGCAGACGATGAAG | | | | |
| CAAAGGATGATCCAGGTGTTCTGGTACACAGTTGTAATTTTGACCATGGACTTTGTGGATGGATCAGG | | | | |
| GAGAAGACAATGACTTGCACTGGGAACCAATCAGGGACCCAGCAGGTGGACAATAATCTGACAGTGTC | | | | |
| GGCAGCCAAAGCCCCAGGGGGAAAAGCTGCACGCTTGGTGCTACCTCTCGGCCGCCTCATGCATTCAG | | | | |
| GGGACCTGTGCCTGTCATTCAGGCACAAGGTGACGGGGCTGCACTCTGGCACACTCCAGGTGTTTGTG | | | | |
| AGAAAACACGGTGCCCACGGAGCAGCCCTGTGGGGAAGAAATGGTGGCCATGGCTGGAGGCAAACACA | | | | |
| GATCACCTTGCGAGGGGCTGACATCAAGAGCGTCGTCTTCAAAGGTGAAAAAAGGCGTGGTCACACTG | | | | |
| GGGAGATTGGATTAGATGATGTGAGCTTGAAAAAAGGCCACTGCTCTGAAGAAACGCGTCGACGGC | | | | |
| | NOV3d, 250095779 | SEQ ID NO: 12 | 588 aa | MW at 64534.3kD |
| | Protein Sequence | | | |

| TGSMDFLLALVLVSSLYLQAAAEFDGRWPRQIVSSIGLCRYGGRIDCCWGWARQSWGQCQPFYVLRQR | | |
|---|---|---|
| IARIRCQLKAVCQPRCKHGECIGPNKCKCHPGYAGKTCNQDLNECGLKPRPCKHRCMNTYGSYKCYCL | | |
| NGYMLMPDGSCSSALTCSMANCQYGCDVVKGQIRCQCPSPGLHLAPDGRTCVDVDECATGRASCPRFR | | |
| QCVNTFGSYICKCHKGFDLMYIGGKYQCHDIDECSLGQYQCSSFARCYNVRGSYKCKCKEGYQGDGLT | | |
| CVYIPKVMIEPSGPIHVPKGNGTILKGDTGNNNWIPDVGSTWWPPKTPYIPPIITNRPTSKPTTRPTP | | |
| KPTPIPTPPPPPPLPTELRTPLPPTTPERPTTGLTTIAPAASTPPGGITVDNRVQTDPQKPRGDVFIP | | |
| RQPSNDLFEIFEIERGVSADDEAKDDPGVLVHSCNFDHGLCGWIREKDNDLHWEPIRDPAGGQYLTVS | | |
| AAKAPGGKAARLVLPLGRLMHSGDLCLSFRHKVTGLHSGTLQVFVRKHGAHGAALWGRNGGHGWRQTQ | | |
| ITLRGADIKSVVFKGEKRRGHTGEIGLDDVSLKKGHCSEERVDG | | |
| NOV3e, 250095794 | SEQ ID NO: 13 | 1657 bp |
| DNA Sequence | ORF Start: at 2 | ORF Stop: end of sequence |

| CACCGGATCCGAGTTCGACGGGAGGTGGCCCAGGCAAATAGTGTCATCGATTGGCCTATGTCGTTATG | | | | |
|---|---|---|---|---|
| GTGGGAGGATTGACTGCTGCTGGGGCTGGGCTCGCCAGTCTTGGGGACAGTGTCAGCCTGTGTGCCAA | | | | |
| CCACGATGCAAACATGGTGAATGTATCGGGCCAAACAAGTGCAAGTGTCATCCTGGTTATGCTGGAAA | | | | |
| AACCTGTAATCAAGATCTAAATGAGTGTGGCCTGAAGCCCCGGCCCTGTAAGCACAGGTGCATGAACA | | | | |
| CTTACGGCAGCTACAAGTGCTACTGTCTCAACGGATATATGCTCATGCCGGATGGTTCCTGCTCAAGT | | | | |
| GCCCTGACCTGCTCCATGGCAAACTGTCAGTATGGCTGTGATGTTGTTAAAGGACAAATACGGTGCCA | | | | |
| GTGCCCATCCCCTGGCCTGCACCTGGCTCCTGATGGGAGGACCTGTGTAGATGTTGATGAATGTGCTA | | | | |
| CAGGAAGAGCCTCCTGCCCTAGATTTAGGCAATGTGTCAACACTTTTGGGAGCTACATCTGCAAGTGT | | | | |
| CATAAAGGCTTCGATCTCATGTATATTGGAGGCAAATATCAATGTCATGACATAGACGAATGCTCACT | | | | |
| TGGTCAGTATCAGTGCAGCAGCTTTGCTCGATGTTATAACGTACGTGGGTCCTACAAGTGCAAATGTA | | | | |
| AAGAAGGATACCAGGGTGATGGACTGACTTGTGTGTATATCCCAAAAGTTATGATTGAACCTTCAGGT | | | | |
| CCAATTCATGTACCAAAGGGAAATGGTACCATTTTAAAGGGTGACACAGGAAATAATAATTGGATTCC | | | | |
| TGATGTTGGAAGTACTTGGTGGCCTCCGAAGACACCATATATTCCTCCTATCATTACCAACAGGCCTA | | | | |
| CTTCTAAGCCAACAACAAGACCTACACCAAAGCCAACACCAATTCCTACTCCACCACCACCACCACCC | | | | |
| CTGCCAACAGAGCTCAGAACACCTCTACCACCTACAACCCCAGAAAGGCCAACCACCGGACTGACAAC | | | | |
| TATAGCACCAGCTGCCAGTACACCTCCAGGAGGGATTACAGTTGACAACAGGGTACAGACAGACCCTC | | | | |
| AGAAACCCAGAGGAGATGTGTTCATTCCACGGCAACCTTCAAATGACTTGTTTGAAATATTTGAAATA | | | | |
| GAAAGAGGAGTCAGTGCAGACGATGAAGCAAAGGATGATCCAGGTGTTCTGGTACACAGTTGTAATTT | | | | |
| TGACCATGGACTTTGTGGATGGATCAGGGAGAAAGACAATGACTTGCACTGGGAACCAATCAGGGACC | | | | |
| CAGCAGGTGGACAATATCTGACAGTGTCGGCAGCCAAAGCCCCAGGGGGAAAAGCTGCACGCTTGGTG | | | | |
| CTACCTCTCGGCCGCCTCATGCATTCAGGGGACCTGTGCCTGTCATTCAGGCACAAGGTGACGGGGCT | | | | |
| GCACTCTGGCACACTCCAGGTGTTTGTGAGAAAACACGGTGCCCACGGAGCAGCCCTGTGGGGAAGAA | | | | |
| ATGGTGGCCATGGCTGGAGGCAAACACAGATCACCTTGCGAGGGGCTGACATCAAGAGCGTCGTCTTC | | | | |
| AAAGGTGAAAAAAGGCGTGGTCACACTGGGGAGATTGGATTAGATGATGTGAGCTTGAAAAAAGGCCA | | | | |
| CTGCTCTGAAGAACGCGTCGACGGC | | | | |
| | NOV3e, 250095794 | SEQ ID NO: 14 | 552 aa | MW at 60397.3kD |
| | Protein Sequence | | | |

| TGSEFDGRWPRQTVSSIGLCRYGGRIDCCWGWARQSWGQCQPVCQPRCKHGECIGPNKCKCHPGYAGK | | |
|---|---|---|
| TCNQDLNECGLKPRPCKHRCMNTYGSYKCYCLNGYMLMPDGSCSSALTCSMANCQYGCDVVKGQIRCQ | | |
| CPSPGLHLAPDGRTCVDVDECATGRASCPRFRQCVNTFGSYICKCHKGFDLMYIGGKYQCHDIDECSL | | |
| GQYQCSSFARCYNVRGSYKCKCKEGYQGDGLTCVYIPKVMIEPSGPIHVPKGNGTILKGDTGNNNWIP | | |
| DVGSTWWPPKTPYIPPITTNRPTSKPTTRPTPKPTPIPTPPPPPPLPTELRTPLPPTTPERPTTGLTT | | |
| IAPAASTPPGGITVDNRVQTDPQKPRGDVFIPRQPSNDLFEIFEIERGVSADDEAKDDPGVLVHSCNF | | |
| DHGLCGWIREKDNDLHWEPIRDPAGGQYLTVSAAKAPGGKAARLVLPLGRLMHSGDLCLSFRHKVTGL | | |
| HSGTLQVFVRKHGAHGAALWGRNGGHGWRQTQITLRGADIKSVVFKGEKRRGHTGEIGLDDVSLKKGH | | |
| CSEERVDG | | |
| NOV3f, 250095734 | SEQ ID NO: 15 | 1717 bp |
| DNA Sequence | ORF Start: at 2 | ORF Stop: end of sequence |

| CACCGGATCCATGGATTTTCTCCTGGCGCTGGTGCTGGTATCCTCGCTCTACCTGCAGGCGGCCGCCG | | | | |
|---|---|---|---|---|
| AGTTCGACGGGAGTAGGTGGCCCAGGCAAATAGTGTCATCGATTGGCCTATGTCGTTATGGTGGGAGG | | | | |
| ATTGACTGCTGCTGGGGCTGGGCTCGCCAGTCTTGGGGACAGTGTCAGCCTGTGTGCCAACCACGATG | | | | |
| CAAACATGGTGAATGTATCGGGCCAAACAAGTGCAAGTGTCATCCTGGTTATGCTGGAAAAACCTGTA | | | | |
| ATCAAGATCTAAATGAGTGTGGCCTGAAGCCCCGGCCCTGTAAGCACAGGTGCATGAACACTTACGGC | | | | |
| AGCTACAAGTGCTACTGTCTCAACGGATATATGCTCATGCCGGATGGTTCCTGCTCAAGTGCCCTGAC | | | | |
| CTGCTCCATGGCAAACTGTCAGTATGGCTGTGATGTTGTTAAAGGACAAATACGGTGCCAGTAACCAT | | | | |
| CCCCTGGCCTGCACCTGGCTCCTGATGGGAGGACCTGTGTAGATGTTGATGAATGTGCTACAGGAAGA | | | | |
| GCCTCCTGCCCTAGATTTAGGCAATGTGTCAACACTTTTGGGAGCTACATCTGCAAGTGTCATAAAGG | | | | |
| CTTCGATCTCATGTATATTGGAGGCAAATATCAATGTCATGACATAGACGAATGCTCACTTGGTCAGT | | | | |
| ATCAGTGCAGCAGCTTTGCTCGATGTTATAACGTACGTGGGTCCTACAAGTGCAAATGTAAAGAAGGA | | | | |
| TACCAGGGTGATGGACTGACTTGTGTGTATATCCCAAAAGTTATGATTGAACCTTCAGGTCCAATTCA | | | | |
| TGTACCAAAGGGAAATGGTACCATTTTAAAGGGTGACACAGGAAATAATAATTGGATTCCTGATGTTG | | | | |
| GAAGTACTTGGTGGCCTCCGAAGACACCATATATTCCTCCTATCATTACCAACAGGCCTACTTCTAAG | | | | |
| CCAACAACAAGACCTACACCAAAGCCAACACCAATTCCTACTCCACCACCACCACCACCCCTGCCAAC | | | | |
| AGAGCTCAGAACACCTCTACCACCTACAACCCCAGAAAGGCCAACCACCGGACTGACAACTATAGCAC | | | | |
| CAGCTGCCAGTACACCTCCAGGAGGGATTACAGTTGACAACAGGGTACAGACAGACCCTCAGAAACCC | | | | |
| AGAGGAGATGTGTTCATTCCACGGCAACCTTCAAATGACTTGTTTGAAATATTTGAAATAGAAAGAGG | | | | |
| AGTCAGTGCAGACGATGAAGCAAAGGATGATCCAGGTGTTCTGGTACACAGTTGTAATTTTGACCATG | | | | |
| GACTTTGTGGATGGATCAGGGAGAAAGACAATGACTTGCACTGGGAACCAATCAGGGACCCAGCAGGT | | | | |
| GGACAATATCTGACAGTGTCGGCAGCCAAAGCCCCAGGGGGAAAAGCTGCACGCTTGGTGCTACCTCT | | | | |
| CGGCCGCCTCATGCATTCAGGGGACCTGTGCCTGTCATTCAGGCACAAGGTGACGGGGCTGCACTCTG | | | | |
| GCACACTCCAGGTGTTTGTGAGAAAACACGGTGCCCACGGAGCAGCCCTGTGGGGAAGAAATGGTGGC | | | | |
| CATGGCTGGAGGCAAACACAGATCACCTTGCGAGGGGCTGACATCAAGAGCGTCGTCTTCAAAGGTGA | | | | |
| IAAGAACGCGTCGACGGC | | | | |
| | NOV3f, 250095734 | SEQ ID NO: 16 | 572 aa | MW at 62504.8kD |
| | Protein Sequence | | | |

| TGSMDFLLALVLVSSLYLQAAAEFDGSRWPRQIVSSIGLCRYGGRIDCCWGWARQSWGQCQPVCQPRC | | |
|---|---|---|
| KHGECIGPNKCKCHPGYAGKTCNQDLNECGLKPRPCKHRCMNTYGSYKCYCLNGYMLMPDGSCSSALT | | |
| CSMANCQYGCDVVKGQIRCQCPSPGLHLAPDGRTCVDVDECATGRASCPRFRQCVNTFGSYICKCHKG | | |
| FDLMYIGGKYQCHDIDECSLGQYQCSSFARCYNVRGSYKCKCKEGYQGDGLTCVYIPKVMIEPSGPIH | | |
| VPKGNGTTLKGDTGNNNWIPDVGSTWWPPKTPYTPPIITNRPTSKPTTRPTPKPTPTPTPPPPPPLPT | | |
| ELRTPLPPTTPERPTTGLTTIAPAASTPPGGITVDNRVQTDPQKPRGDVFIPRQPSNDLFEIFEIERG | | |
| VSADDEAKDDPGVLVHSCNFDHGLCGWIREKDNDLNWEPIRDPAGGQYLTVSAAKAPGGKAARLVLPL | | |
| GRLMHSGDLCLSFRBKVTGLHSGTLQVFVRKHGAHGAALWGRNGGHGWRQTQITLRGADIKSVVFKGE | | |
| KRRGHTGEIGLDDVSLKKGHCSEERVDG | | |
| NOV3g, 250095811 | SEQ ID NO: 17 | 613 bp |
| DNA Sequence | ORF Start: at 2 | ORF Stop: end of sequence |

| CACCGGATCCTGTCAGCCTGTGTGCCAACCACGATGCAAACATGGTGAATGTATCGGGCCAAACAAGT | | | | |
|---|---|---|---|---|
| GCAAGTGTCATCCTGGTTATGCTGGAAAAACCTGTAATCAAGATCTAAATGAGTGTGGCCTGAAGCCC | | | | |
| CGGCCCTGTAAGCACAGGTGCATGAACACTTACGGCAGCTACAAGTGCTACTGTCTCAACGGATATAT | | | | |
| GCTCATGCCGGATGGTTCCTGCTCAAGTGCCCTGACCTGCTCCATGGCAAACTGTCAGTATGGCTGTG | | | | |
| ATGTTGTTAAAGGACAAATACGGTGCCAGTGCCCATCCCCTGGCCTGCAGCTGGCTCCTGATGGGAGG | | | | |
| ACCTGTGTAGATGTTGATGAATGTGCTACAGGAAGAGCCTCCTGCCCTAGATTTAGGCAATGTGTCAA | | | | |
| CACTTTTGGGAGCTACATCTGCAAGTGTCATAAAGGCTTCGATCTCATGTATATTGGAGGCAAATATC | | | | |
| AATGTCATGACATAGACGAATGCTCACTTGGTCAGTATCAGTGCAGCAGCTTTGCTCGATGTTATAAC | | | | |
| GTACGTGGGTCCTACAAGTGCAAATGTAAAGAAGGATACCAGGGTGATGGACTGACTTGTGTCGACGG | | | | |
| C | | | | |
| | Nov3g, 250095811 | SEQ ID NO: 18 | 204 aa | MW at 22351 .4kD |
| | Protein Sequence | | | |

| TGSCQPVCQPRCKHGECIGPNKCKCHPGYAGKTCNQDLNECGLKPRPCKHRCMNTYGSYKCYCLNGYM | | | |
|---|---|---|---|
| LMPDGSCSSALTCSMANCQYGCDVVKGQIRCQCPSPGLQLAPDGRTCVDVDECATGRASCPRFRQCVN | | | |
| TFGSYICKCHKGFDLMYIGGKYQCHDIDECSLGQYQCSSFARCYNVRGSYKCKCKEGYQGDGLTCVDG | | | |
| | NOV3h, 250095799 | SEQ ID NO: 19 | 613 bp |
| | DNA Sequence | ORF Start: at 2 | ORF Stop: end of sequence |

| CACCGGATCCTGTCAGCCTGTGTGCCAACCACGATGCAAACATGGTGAATGTATCGGGCCAAACAAGT | | | | |
|---|---|---|---|---|
| GCAAGTGTCATCCTGGTTATGCTGGAAAAACCTGTAATCAAGATCTAAATGAGTGTGGCCTGAAGCCC | | | | |
| CGGCCCTGTAAGCACAGGTGCATGAACACTTACGGCAGCTACAAGTGCTACTGTCTCAACGGATATAT | | | | |
| GCTCATGCCGGATGGTTCCTGCTCAAGTGCCCTGACCTGCTCCATGGCAAACTGTCAGTATGGCTGTG | | | | |
| ATGTTGTTAAAGGACAAATACGGTGCCAGTGCCCATCCCCTGGCCTGCACCTGGCTCCTGATGGGAGG | | | | |
| ACCTGTGTAGATGTTGATGAATGTGCTACAGGAAGAGCCTCCTGCCCTAGATTTAGGCAATGTGTCAA | | | | |
| CACTTTTGGGAGCTACATCTGCAAGTGTCATAAAGGCTTCGATCTCATGTATATTGGAGGCAAATATC | | | | |
| AATGTCATGACATAGACGAATGCTCACTTGGTCAGTATCAGTGCAGCAGCTTTGCTCGATGTTATAAC | | | | |
| GTACGTGGGTCCTACAAGTGCAAATGTAAAGAAGGATACCAGGGTGATGGACTGACTTGTGTCGACGG | | | | |
| C | | | | |
| | NOV3h, 250095799 | SEQ ID NO: 20 | 204 aa | MW at 22360.4kD |
| | Protein Sequence | | | |

| GSCQPVCQPRCKHGECIGPNKCKCHPGYAGKTCNQDLNECGLKPRPCKHRCMNTYGSYKCYCLNGYM | | | |
|---|---|---|---|
| LMPDGSCSSALTCSMANCQYGCDWKGQIRCQCPSPGLHLAPDGRTCVDVDECATGRASCPRFRQCVN | | | |
| FGSYICKCHKGFDLMYIGGKYQCHDIDECSLGQYQCSSFARCYNVRGSYKCKCKEGYQGDGLTCVDG | | | |
| NOV3i, SNP13377609 of | SEQ ID NO: 21 | | 1882bp |
| CG50253-01, DNA Sequence | ORF Start: ATG at 243 | | ORF Stop: TAA at 1851 |
| | SNP Pos: 63 | | SNP Change: G to A |
| GGACACTGACATGGACTGAAGGAGTAGAAAAGAAGGGAGCGGGAGGGGGCTCCGGGCGCCGCACAGCA | | | |
| GACCTACTCCGGCCGCGCGCCTCGCCGCTGTCCTCCGGGAGCGGCAGCAGTAGCCCGGGCGGCGAGGG | | | |
| CTGGGGGTTCCTCGAGACTCTCAGAGGGGCGCCTCCCATCGGCGCCCACCACCCCAACCTGTTCCTCG | | | |
| CGCGCCACTGCGCTGCGCCCCAGGACCCGCTGCCCAACATGGATTTTCTCCTGGCGCTGGTGCTGGTA | | | |
| TCCTCGCTCTACCTGCAGGCGGCCGCCGAGTTCGACGGGAGGTGGCCCAGGCAAATAGTGTCATCGAT | | | |
| TGGCCTATGTCGTTATGGTGGGAGGATTGACTGCTGCTGGGGCTGGGCTCGCCAGTCTTGGGGACAGT | | | |
| GTCAGCCTGTGTGCCAACCACGATGCAAACATGGTGAATGTATCGGGCCAAACAAGTGCAAGTGTCAT | | | |
| CCTGGTTATGCTGGAAAAACCTGTAATCAAGATCTAAATGAGTGTGGCCTGAAGCCCCGGCCCTGTAA | | | |
| GCACAGGTGCATGAACACTTACGGCAGCTACAAGTGCTACTGTCTCAACGGATATATGCTCATGCCGG | | | |
| ATGGTTCCTGCTCAAGTGCCCTGACCTGCTCCATGGCAAACTGTCAGTATGGCTGTGATGTTGTTAAA | | | |
| GGACAAATACGGTGCCAGTGCCCATCCCCTGGCCTGCAGCTGGCTCCTGATGGGAGGACCTGTGTAGA | | | |
| TGTTGATGAATGTGCTACAGGAAGAGCCTCCTGCCCTAGATTTAGGCAATGTGTCAACACTTTTGGGA | | | |
| GCTACATCTGCAAGTGTCATAAAGGCTTCGATCTCATGTATATTGGAGGCAAATATCAATGTCATGAC | | | |
| ATAGACGAATGCTCACTTGGTCAGTATCAGTGCAGCAGCTTTGCTCGATGTTATAACATACGTGGGTC | | | |
| CTACAAGTGCAAATGTAAAGAAGGATACCAGGGTGATGGACTGACTTGTGTGTATATCCCAAAAGTTA | | | |
| TGATTGAACCTTCAGGTCCAATTCATGTACCAAAGGGAAATGGTACCATTTTAAAGGGTGACACAGGA | | | |
| AATAATAATTGGATTCCTGATGTTGGAAGTACTTGGTGGCCTCCGAAGACACCATATATTCCTCCTAT | | | |
| CATTACCAACAGGCCTACTTCTAAGCCAACAACAAGACCTACACCAAAGCCAACACCAATTCCTACTC | | | |
| CACCACCACCACCACCCCTGCCAACAGAGCTCAGAACACCTCTACCACCTACAACCCCAGAAAGGCCA | | | |
| ACCACCGGACTGACAACTATAGCACCAGCTGCCAGTACACCTCCAGGAGGGATTACAGTTGACAACAG | | | |
| GGTACAGACAGACCCTCAGAAACCCAGAGGAGATGTGTTCAGTGTTCTGGTACACAGTTGTAATTTTG | | | |
| ACCATGGACTTTGTGGATGGATCAGGGAGAAAGACAATGACTTGCACTGGGAACCAATCAGGGACCCA | | | |
| GCAGGTGGACAATATCTGACAGTGTCGGCAGCCAAAGCCCCAGGGGGAAAAGCTGCACGCTTGGTGCT | | | |
| ACCTCTCGGCCGCCTCATGCATTCAGGGGACCTGTGCCTGTCATTCAGGCACAAGGTGACGGGGCTGC | | | |
| ACTCTGGCACACTCCAGGTGTTTGTGAGAAAACACGGTGCCCACGGAGCAGCCCTGTGGGGAAGAAAT | | | |
| GGTGGCCATGGCTGGAGGCAAACACAGATCACCTTGCGAGGGGCTGACATCAAGAGCGTCGTCTTCAA | | | |
| AGGTGAAAAAAGGCGTGGTCACACTGGGGAGATTGGATTAGATGATGTGAGCTTGAAAAAAGGCCACT | | | |
| GCTCTGAAGAACGCTAACAACTCCAGAACTAACAATGAACTCCTAA | | | |
| NOV3i, SNP13377609 of | SEQ ID NO: 22 | 536 aa | MW at 58650.8kD |
| CG50253-01, Protein Sequence | | | SNP Change: no change |
| MDFLLALVLVSSLYLQAAAEFDGRWPRQIVSSIGLCRYGGRIDCCWGWARQSWGQCQPVCQPRCKHGE | | | |
| CIGPNKCKCHPGYAGKTCNQDLNECGLKPRPCKHRCMNTYGSYKCYCLNGYMLMPDGSCSSALTCSMA | | | |
| NCQYGCDVVKGQIRCQCPSPGLQTAPDGRTCVDVDECATGRASCPRFRQCVNTFGSYICKCHKGFDLM | | | |
| YIGGKYQCHDIDECSLGQYQCSSFARCYNIRGSYKCKCKEGYQGDGLTCVYIPKVMIEPSGPIHVPKG | | | |
| NGTILKGDTGNNNWIPDVGSTWWPPKTPYIPPIITNRPTSKPTTRPTPKPTPIPTPPPPPPLPTELRT | | | |
| PLPPTTPERPTTGLTTIAPAASTPPGGITVDNRVQTDPQKPRGDVFSVLVHSCNFDHGLCGWIREKDN | | | |
| DLHWEPIRDPAGGQYLTVSAAKAPGGKAARLVLPLGRLMHSGDLCLSFRHKVTGLHSGTLQVFVRKHG | | | |
| IAHGAALWGRNGGHGWRQTQITLRGADIKSVVFKGEKRRGHTGEIGLDDVSLKKGHCSEER | | | |
| NOV3j, SNP13373929 of | SEQ ID NO: 23 | | 1882 bp |
| CG50253-O1, DNA Sequence | ORF Start: ATG at 243 | | ORE Stop: TM at 1851 |
| | SNP Pos: 226 | | SNP Change: A to G |
| GGACACTGACATGGACTGAAGGAGTAGAAAAGAAGGGAGCGGGAGGGGGCTCCGGGCGCCGCGCAGCA | | | |
| GACCTACTCCGGCCGCGCGCCTCGCCGCTGTCCTCCGGGAGCGGCAGCAGTAGCCCGGGCGGCGAGGG | | | |
| CTGGGGGTTCCTCGAGACTCTCAGAGGGGCGCCTCCCATCGGCGCCCACCACCCCAACCTGTTCCTCG | | | |
| CGCGCCACTGCGCTGCGCCCCGGGACCCGCTGCCCAACATGGATTTTCTCCTGGCGCTGGTGCTGGTA | | | |
| TCCTCGCTCTACCTGCAGGCGGCCGCCGAGTTCGACGGGAGGTGGCCCAGGCAAATAGTGTCATCGAT | | | |
| TGGCCTATGTCGTTATGGTGGGAGGATTGACTGCTGCTGGGGCTGGGCTCAACAGTCTTAAGGACAGT | | | |
| GTCAGCCTGTGTGCCAACCACGATGCAAACATGGTGAATGTATCGGGCCAAACAAGTGCAAGTGTCAT | | | |
| CCTGGTTATGCTGGAAAAACCTGTAATCAAGATCTAAATGAGTGTGGCCTGAAGCCCCGGCCCTGTAA | | | |
| GCACAGGTGCATGAACACTTACGGCAGCTACAAGTGCTACTGTCTCAACGGATATATGCTCATGCCGG | | | |
| ATGGTTCCTGCTCAAGTGCCCTGACCTGCTCCATGGCAAACTGTCAGTATGGCTGTGATGTTGTTAAA | | | |
| GGACAAATACGGTGCCAGTGCCCATCCCCTGGCCTGCAGCTGGCTCCTGATGGGAGGACCTGTGTAGA | | | |
| TGTTGATGAATGTGCTACAGGAAGAGCCTCCTGCCCTAGATTTAGGCAATGTGTCAACACTTTTGGGA | | | |
| GCTACATCTGCAAGTGTCATAAAGGCTTCGATCTCATGTATATTGGAGGCAAATATCAATGTCATGAC | | | |
| ATAGACGAATGCTCACTTGGTCAGTATCAGTGCAGCAGCTTTGCTCGATGTTATAACATACGTGGGTC | | | |
| CTACAAGTGCAAATGTAAAGAAGGATACCAGGGTGATGGACTGACTTGTGTGTATATCCCAAAAGTTA | | | |
| TGATTGAACCTTCAGGTCCAATTCATGTACCAAAGGGAAATGGTACCATTTTAAAGGGTGACACAGGA | | | |
| AATAATAATTGGATTCCTGATGTTGGAAGTACTTGGTGGCCTCCGAAGACACCATATATTCCTCCTAT | | | |
| CATTACCAACAGGCCTACTTCTAAGCCAACAACAAGACCTACACCAAAGCCAACACCAATTCCTACTC | | | |
| CACCACCACCACCACCCCTGCCAACAGAGCTCAGAACACCTCTACCACCTACAACCCCAGAAAGGCCA | | | |
| ACCACCGGACTGACAACTATAGCACCAGCTGCCAGTACACCTCCAGGAGGGATTACAGTTGACAACAG | | | |
| GGTACAGACAGACCCTCAGAAACCCAGAGGAGATGTGTTCAGTGTTCTGGTACACAGTTGTAATTTTG | | | |
| ACCATGGACTTTGTGGATGGATCAGGGAGAAAGACAATGACTTGCACTGGGAACCAATCAGGGACCCA | | | |
| GCAGGTGGACAATATCTGACAGTGTCGGCAGCCAAAGCCCCAGGGGGAAAAGCTGCACGCTTGGTGCT | | | |
| ACCTCTCGGCCGCCTCATGCATTCAGGGGACCTGTGCCTGTCATTCAGGCACAAGGTGACGGGGCTGC | | | |
| ACTCTGGCACACTCCAGGTGTTTGTGAGAAAACACGGTGCCCACGGAGCAGCCCTGTGGGGAAGAAAT | | | |
| GGTGGCCATGGCTGGAGGCAAACACAGATCACCTTGCGAGGGGCTGACATCAAGAGCGTCGTCTTCAA | | | |
| AGGTGAAAAAAGGCGTGGTCACACTGGGGAGATTGGATTAGATGATGTGAGCTTGAAAAAAGGCCACT | | | |
| GCTCTGAAGAACGCTAACAACTCCAGAACTAACAATGAACTCCTAA | | | |
| NoV3j, SNP13373929 of | SEQ ID NO: 24 | 536 aa | 1MW at 58650.8kD |
| CG50253-01, Protein Sequence | | | SNP Change: no change |

| MDFLLALVLVSSLYLQAAAEFDGRWPRQIVSSIGLCRYGGRIDCCWGWARQSWGQCQPVCQPRCKHGE | | |
|---|---|---|
| CIGPNKCKCHPGYAGKTCNQDLNECGLKPRPCKHRCMNTYGSYKCYCLNGYMLMPDGSCSSALTCSMA | | |
| NCQYGCDVVKGQIRCQCPSPGLQLAPDGRTCVDVDECATGRASCPRFRQCVNTFGSYICKCHKGFDLM | | |
| YIGGKYQCHDIDECSLGQYQCSSFARCYNIRGSYKCKCKEGYQGDGLTCVYIPKVMIEPSGPIHVPKG | | |
| NGTILKGDTGNNNWIPDVGSTWWPPKTPYIPPIITNRPTSKPTTRPTPKPTPIPTPPPPPPLPTELRT | | |
| PLPPTTPERPTTGLTTIAPAASTPPGGITVDNRVQTDPQKPRGDVFSVLVHSCNFDHGLCGWIREKDN | | |
| DLHWEPIRDPAGGQYLTVSAAKAPGGKAARLVLPLGRLMHSGDLCLSFRHKVTGLHSGTLQVFVRKHG | | |
| AHGAALWGRNGGHGWRQTQITLRGADIKSVVFKGEKRRGHTGEIGLDDVSLKKGHCSEER | | |
| NOV3k, SNP13380272 of | SEQ ID NO: 25 | 1882 bp |
| CG50253-01, DNA Sequence | ORF Start: ATG at 243 | ORF Stop: TM at 1851 |
| | SNP Pos: 719 | SNP Change: G to C |

| GGACACTGACATGGACTGAAGGAGTAGAAAAGAAGGGAGCGGGAGGGGGCTCCGGGCGCCGCGCAGCA | | | |
|---|---|---|---|
| GACCTACTCCGGCCGCGCGCCTCGCCGCTGTCCTCCGGGAGCGGCAGCAGTAGCCCGGGCGGCGAGGG | | | |
| CTGGGGGTTCCTCGAGACTCTCAGAGGGGCGCCTCCCATCGGCGCCCACCACCCCAACCTGTTCCTCG | | | |
| CGCGCCACTGCGCTGCGCCCCAGGACCCGCTGCCCAACATGGATTTTCTCCTGGCGCTGGTGCTGGTA | | | |
| TCCTCGCTCTACCTGCAGGCGGCCGCCGAGTTCGACGGGAGGTGGCCCAGGCAAATAGTGTCATCGAT | | | |
| TGGCCTATGTCGTTATGGTGGGAGGATTGACTGCTGCTGGGGCTGGGCTCGCCAGTCTTGGGGACAGT | | | |
| GTCAGCCTGTGTGCCAACCACGATGCAAACATGGTGAATGTATCGGGCCAAACAAGTGCAAGTGTCAT | | | |
| CCTGGTTATGCTGGAAAAACCTGTAATCAAGATCTAAATGAGTGTGGCCTGAAGCCCCGGCCCTGTAA | | | |
| GCACAGGTGCATGAACACTTACGGCAGCTACAAGTGCTACTGTCTCAACGGATATATGCTCATGCCGG | | | |
| ATGGTTCCTGCTCAAGTGCCCTGACCTGCTCCATGGCAAACTGTCAGTATGGCTGTGATGTTGTTAAA | | | |
| GGACAAATACGGTGCCAGTGCCCATCCCCTGGCCTGCACCTGGCTCCTGATGGGAGGACCTGTGTAGA | | | |
| TGTTGATGAATGTGCTACAGGAAGAGCCTCCTGCCCTAGATTTAGGCAATGTGTCAACACTTTTGGGA | | | |
| GCTACATCTGCAAGTGTCATAAAGGCTTCGATCTCATGTATATTGGAGGCAAATATCAATGTCATGAC | | | |
| ATAGACGAATGCTCACTTGGTCAGTATCAGTGCAGCAGCTTTGCTCGATGTTATAACATACGTGGGTC | | | |
| CTACAAGTGCAAATGTAAAGAAGGATACCAGGGTGATGGACTGACTTGTGTGTATATCCCAAAAGTTA | | | |
| TGATTGAACCTTCAGGTCCAATTCATGTACCAAAGGGAAATGGTACCATTTTAAAGGGTGACACAGGA | | | |
| AATAATAATTGGATTCCTGATGTTGGAAGTACTTGGTGGCCTCCGAAGACACCATATATTCCTCCTAT | | | |
| CATTACCAACAGGCCTACTTCTAAGCCAACAACAAGACCTACACCAAAGCCAACACCAATTCCTACTC | | | |
| CACCACCACCACCACCCCTGCCAACAGAGCTCAGAACACCTCTACCACCTACAACCCCAGAAAGGCCA | | | |
| ACCACCGGACTGACAACTATAGCACCAGCTGCCAGTACACCTCCAGGAGGGATTACAGTTGACAACAG | | | |
| GGTACAGACAGACCCTCAGAAACCCAGAGGAGATGTGTTCAGTGTTCTGGTACACAGTTGTAATTTTG | | | |
| ACCATGGACTTTGTGGATGGATCAGGGAGAAAGACAATGACTTGCACTGGGAACCAATCAGGAACCCA | | | |
| GCAGGTGGACAATATCTGACAGTGTCGGCAGCCAAAGCCCCAGGGGGAAAAGCTGCACGCTTGGTGCT | | | |
| ACCTCTCGGCCGCCTCATGCATTCAGGGGACCTGTGCCTGTCATTCAGGCACAAGGTGACGGGGCTGC | | | |
| ACTCTGGCACACTCCAGGTGTTTGTGAGAAAACACGGTGCCCACGGAGCAGCCCTGTGGGGAAGAAAT | | | |
| GGTGGCCATGGCTGGAGGCAAACACAGATCACCTTGCGAGGGGCTGACATCAAGAGCGTCGTCTTCAA | | | |
| AGGTGAAAAAAGGCGTGGTCACACTGGGGAGATTGGATTAGATGATGTGAGCTTGAAAAAAGGCCACT | | | |
| GCTCTGAAGAACGCTAACAACTCCAGAACTAACAATGAACTCCTAA | | | |
| NOV3k, SNP13380272 of | SEQ ID NO: 26 | 536 aa | MW at 58659.8kD |
| CG50253-01, Protein Sequence | SNP Pos: 159 | SNP Change: Gln to His | |

| MDFLLALVLVSSLYLQAAAEFDGRWPRQIVSSIGLCRYGGRIDCCWGWARQSWGQCQPVCQPRCKHGE | | |
|---|---|---|
| CIGPNKCKCHPGYAGKTCNQDLNECGLKPRPCKHRCMNTYGSYKCYCLNGYMLMPDGSCSSALTCSMA | | |
| NCQYGCDVVKGQIRCQCPSPGLHLAPDGRTCVDVDECATGRASCPRFRQCVNTFGSYICKCHKGFDLM | | |
| YIGGKYQCHDIDECSLGQYQCSSFARCYNIRGSYKCKCKEGYQGDGLTCVYIPKVMIEPSGPIHVPKG | | |
| NGTILKGDTGNNNWIPDVGSTWWPPKTPYIPPIITNRPTSKPTTRPTPKPTPIPTPPPPPPLPTELRT | | |
| PLPPTTPERPTTGLTTIAPAASTPPGGITVDNRVQTDPQKPRGDVFSVLVHSCNFDHGLCGWIREKDN | | |
| DLHWEPIRDPAGGQYLTVSAAKAPGGKAARLVLPLGRLMHSGDLCLSFRHKVTGLHSGTLQVFVRKHG | | |
| AHGAALWGRNGGHGWRQTQITLRGADIKSVVFKGEKRRGHTGEIGLDDVSLKKGHCSEER | | |
| Nov3l, SNP13379745 of | SEQ ID NO: 27 | 1882 bp |
| CG50253-01, DNA Sequence | ORF Start: ATG at 243 | ORF Stop: TAA at 1851 |
| | SNP Pos: 750 | SNP Change: G to A |

| GGACACTGACATGGACTGAAGGAGTAGAAAAGAAGGGAGCGGGAGGGGGCTCCGGGCGCCGCGCAGCA | | | |
|---|---|---|---|
| GACCTACTCCGGCCGCGCGCCTCGCCGCTGTCCTCCGGGAGCGGCAGCAGTAGCCCGGGCGGCGAGGC | | | |
| CTGGGGGTTCCTCGAGACTCTCAGAGGGGCGCCTCCCATCGGCGCCCACCACCCCAACCTGTTCCTCG | | | |
| CGCGCCACTGCGCTGCGCCCCAGGACCCGCTGCCCAACATGGATTTTCTCCTGGCGCTGGTGCTGGTA | | | |
| TCCTCGCTCTACCTGCAGGCGGCCGCCGAGTTCGACGGGAGGTGGCCCAGGCAAATAGTGTCATCGAT | | | |
| TGGCCTATGTCGTTATGGTGGGAGGATTGACTGCTGCTGGGGCTGGGCTCGCCAGTCTTGGGGACAGT | | | |
| GTCAGCCTGTGTGCCAACCACGATGCAAACATGGTGAATGTATCGGGCCAAACAAGTGCAAGTGTCAT | | | |
| CCTGGTTATGCTGGAAAAACCTGTAATCAAGATCTAAATGAGTGTGGCCTGAAGCCCCGGCCCTGTAA | | | |
| GCACAGGTGCATGAACACTTACGGCAGCTACAAGTGCTACTGTCTCAACGGATATATGCTCATGCCGG | | | |
| ATGGTTCCTGCTCAAGTGCCCTGACCTGCTCCATGGCAAACTGTCAGTATGGCTGTGATGTTGTTAAA | | | |
| GGACAAATACGGTGCCAGTGCCCATCCCCTGGCCTGCAGCTGGCTCCTGATGGGAGGACCTGTGTAGA | | | |
| TATTGATGAATGTGCTACAGGAAGAGCCTCCTGCCCTAGATTTAGGCAATGTGTCAACACTTTTGGGA | | | |
| GCTACATCTGCAAGTGTCATAAAGGCTTCGATCTCATGTATATTGGAGGCAAATATCAATGTCATGAC | | | |
| ATAGACGAATGCTCACTTGGTCAGTATCAGTGCAGCAGCTTTGCTCGATGTTATAACATACGTGGGTC | | | |
| CTACAAGTGCAAATGTAAAGAAGGATACCAGGGTGATGGACTGACTTGTGTGTATATCCCAAAAGTTA | | | |
| TGATTGAACCTTCAGGTCCAATTCATGTACCAAAGGGAAATGGTACCATTTTAAAGGGTGACACAGGA | | | |
| AATAATAATTGGATTCCTGATGTTGGAAGTACTTGGTGGCCTCCGAAGACACCATATATTCCTCCTAT | | | |
| CATTACCAACAGGCCTACTTCTAAGCCAACAACAAGACCTACACCAAAGCCAACACCAATTCCTACTC | | | |
| CACCACCACCACCACCCCTGCCAACAGAGCTCAGAACACCTCTACCACCTACAACCCCAGAAAGGCCA | | | |
| ACCACCGGACTGACAACTATAGCACCAGCTGCCAGTACACCTCCAGGAGGGATTACAGTTGACAACAG | | | |
| GGTACAGACAGACCCTCAGAAACCCAGAGGAGATGTGTTCAGTGTTCTGGTACACAGTTGTAATTTTG | | | |
| ACCATGGACTTTGTGGATGGATCAGGGAGAAAGACAATGACTTGCACTGGGAACCAATCAGGGACCCA | | | |
| GCAGGTGGACAATATCTGACAGTGTCGGCAGCCAAAGCCCCAGGGGGAAAAGCTGCACGCTTGGTGCT | | | |
| ACCTCTCGGCCGCCTCATGCATTCAGGGGACCTGTGCCTGTCATTCAGGCACAAGGTGACGGGGCTGC | | | |
| ACTCTGGCACACTCCAGGTGTTTGTGAGAAAACACGGTGCCCACGGAGCAGCCCTGTGGGGAAGAAAT | | | |
| GGTGGCCATGGCTGGAGGCAAACACAGATCACCTTGCGAGGGGCTGACATCAAGAGCGTCGTCTTCAA | | | |
| AGGTGAAAAAAGGCGTGGTCACACTGGGGAGATTGGATTAGATGATGTGAGCTTGAAAAAAGGCCACT | | | |
| GCTCTGAAGAACGCTAACAACTCCAGAACTAACAATGAACTCCTAA | | | |
| NOV3l, SNP13379745 of | SEQ ID NO: 28 | 536 aa | MW at 58664.8kD |
| CG50253-01, Protein Sequence | SNP Pos: 170 | SNP Change: Val to Ile | |

| MDFLLALVLVSSLYLQAAAEFDGRWPRQIVSSIGLCRYGGRIDCCWGWARQSWGQCQPVCQPRCKHGE | | |
|---|---|---|
| CIGPNKCKCHPGYAGKTCNQDLNECGLKPRPCKHRCMNTYGSYKCYCLNGYMLMPDGSCSSALTCSMA | | |
| NCQYGCDVVKGQIRCQCPSPGLQLAPDGRTCVDIDECATGRASCPRFRQCVNTFGSYICKCHKGFDLM | | |
| YIGGKYQCHDIDECSLGQYQCSSFARCYNIRGSYKCKCKEGYQGDGLTCVYIPKVMIEPSGPIHVPKG | | |
| NGTILKGDTGNNNWIPDVGSTWWPPKTPYIPPIITNRPTSKPTTRPTPKPTPIPTPPPPPPLPTELRT | | |
| PLPPTTPERPTTGLTTIAPAASTPPGGITVDNRVQTDPQKPRGDVFSVLVHSCNFDHGLCGWIREKDN | | |
| DLNWEPIRDPAGGQYLTVSAAKAPGGKAARLVLPLGRLMHSGDLCLSFRHKVTGLHSGTLQVFVRKHG | | |
| AHGAALWGRNGGHGWRQTQITLRGADIKSVVFKGEKRRGHTGEIGLDDVSLKKGHCSEER | | |
| NOV3m, SNP13373930 of | SEQ ID NO: 29 | 1882 bp |
| CG50253-01, DNA Sequence | ORF Start: ATG at 243 | ORF Stop: TAA at 1851 |
| | SNP Pos: 942 | SNP Change: A to G |

| GGACACTGACATGGACTGAAGGAGTAGAAAAGAAGGGAGCGGGAGGGGGCTCCGGGCGCCGCGCAGCA | | | |
|---|---|---|---|
| GACCTACTCCGGCCGCGCGCCTCGCCGCTGTCCTCCGGGAGCGGCAGCAGTAGCCCGGGCGGCGAGGG | | | |
| CTGGGGGTTCCTCGAGACTCTCAGAGGGGCGCCTCCCATCGGCGCCCACCACCCCAACCTGTTCCTCG | | | |
| CGCGCCACTGCGCTGCGCCCCAGGACCCGCTGCCCAACATGGATTTTCTCCTGGCGCTGGTGCTGGTA | | | |
| TCCTCGCTCTACCTGCAGGCGGCCGCCGAGTTCGACGGGAGGTGGCCCAGGCAAATAGTGTCATCGAT | | | |
| TGGCCTATGTCGTTATGGTGGGAGGATTGACTGCTGCTGGGGCTGGGCTCGCCAGTCTTGGGGACAGT | | | |
| GTCAGCCTGTGTGCCAACCACGATGCAAACATGGTGAATGTATCGGGCCAAACAAGTGCAAGTGTCAT | | | |
| CCTGGTTATGCTGGAAAAACCTGTAATCAAGATCTAAATGAGTGTGGCCTGAAGCCCCGGCCCTGTAA | | | |
| GCACAGGTGCATGAACACTTACGGCAGCTACAAGTGCTACTGTCTCAACGGATATATGCTCATGCCGG | | | |
| ATGGTTCCTGCTCAAGTGCCCTGACCTGCTCCATGGCAAACTGTCAGTATGGCTGTGATGTTGTTAAA | | | |
| GGACAAATACGGTGCCAGTGCCCATCCCCTGGCCTGCAGCTGGCTCCTGATGGGAGGACCTGTGTAGA | | | |
| TGTTGATGAATGTGCTACAGGAAGAGCCTCCTGCCCTAGATTTAGGCAATGTGTCAACACTTTTGGGA | | | |
| GCTACATCTGCAAGTGTCATAAAGGCTTCGATCTCATGTATATTGGAGGCAAATATCAATGTCATGAC | | | |
| ATAGACGAATGCTCACTTGGTCAGTATCAGTGCAGCAGCTTTGCTCGATGTTATAACGTACGTGGGTC | | | |
| CTACAAGTGCAAATGTAAAGAAGGATACCAGGGTGATGGACTGACTTGTGTGTATATCCCAAAAGTTA | | | |
| TGATTGAACCTTCAGGTCCAATTCATGTACCAAAGGGAAATGGTACCATTTTAAAGGGTGACACAGGA | | | |
| AATAATAATTGGATTCCTGATGTTGGAAGTACTTGGTGGCCTCCGAAGACACCATATATTCCTCCTAT | | | |
| CATTACCAACAGGCCTACTTCTAAGCCAACAACAAGACCTACACCAAAGCCAACACCAATTCCTACTC | | | |
| CACCACCACCACCACCCCTGCCAACAGAGCTCAGAACACCTCTACCACCTACAACCCCAGAAAGGCCA | | | |
| ACCACCGGACTGACAACTATAGCACCAGCTGCCAGTACACCTCCAGGAGGGATTACAGTTGACAACAG | | | |
| GGTACAGACAGACCCTCAGAAACCCAGAGGAGATGTGTTCAGTGTTCTGGTACACAGTTGTAATTTTG | | | |
| ACCATGGACTTTGTGGATGGATCAGGGAGAAAGACAATGACTTGCACTGGGAACCAATCAGGGACCCA | | | |
| GCAGGTGGACAATATCTGACAGTGTCGGCAGCCAAAGCCCCAGGGGGAAAAGCTGCACGCTTGGTGCT | | | |
| ACCTCTCGGCCGCCTCATGCATTCAGGGGACCTGTGCCTGTCATTCAGGCACAAGGTGACGGGGCTGC | | | |
| ACTCTGGCACACTCCAGGTGTTTGTGAGAAAACACGGTGCCCACGGAGCAGCCCTGTGGGGAAGAAAT | | | |
| GGTGGCCATGGCTGGAGGCAAACACAGATCACCTTGCGAGGGGCTGACATCAAGAGCGTCGTCTTCAA | | | |
| AGGTGAAAAAAGGCGTGGTCACACTGGGGAGATTGGATTAGATGATGTGAGCTTGAAAAAAGGCCACT | | | |
| GCTCTGAAGAACGCTAACAACTCCAGAACTAACAATGAACTCCTAA | | | |
| NOV3m, SNP13373930 of | SEQ ID NO: 30 | 536 aa | MW at 58636.8kD |
| CG50253-01, Protein Sequence | SNP Pos: 234 | SNP Change: Ile to Val | |

| | |
|---|---|
| MDFLLALVLVSSLYLQAAAEFDGRWPRQIVSSIGLCRYGGRIDCCWGWARQSWGQCQPVCQPRCKHGE | +TL,42 |
| CIGPNKCKCHPGYAGKTCNQDLNECGLKPRPCKHRCMNTYGSYKCYCLNGYMLMPDGSCSSALTCSMA | |
| NCQYGCDVVKGQIRCQCPSPGLQLAPDGRTCVDVDECATGRASCPRFRQCVNTFGSYICKCHKGFDLM | |
| YIGGKYQCHDIDECSLGQYQCSSFARCYNVRGSYKCKCKEGYQGDGLTCVYIPKVMIEPSGPIHVPKG | |
| NGTILKGDTGNNNWIPDVGSTWWPPKTPYIPPIITNRPTSKPTTRPTPKPTPIPTPPPPPPLPTELRT | |
| PLPPTTPERPTTGLTTIAPAASTPPGGITVDNRVQTDPQKPRGDVFSVLVHSCNFDHGLCGWIREKDN | |
| DLHWEPIRDPAGGQYLTVSAAKAPGGKAARLVLPLGRLMHSGDLCLSFRHKVTGLHSGTLQVFVRKHG | |
| AHGAALWGRNGGHGWRQTQITLRGADIKSVVFKGEKRRGHTGEIGLDDVSLKKGHCSEER | |

A ClustalW comparison of the above protein sequences yields the following sequence alignment shown in Table 3B.

TABLE 3B
Comparison of the NOV3 protein sequences.

| | |
|---|---|
| NOV3a | ---MDFLLALVLVSSLYLQAAAEFDG-RWPRQIVSSIGLCRYGGRIDCCWGWARQSWGQC |
| NOV3b | TGSMDFLLALVLVSSLYLQAAAEFDG-RWPRQIVSSIGLCRYGGRIDCCWGWARQSWGQC |
| NOV3c | TGSMDFLLALVLVSSLYLQAAAEFDG-RWPRQIVSSIGLCRYGGRIDCCWGWARQSWGQC |
| NOV3d | TGSMDFLLALVLVSSLYLQAAAEFDG-RWPRQIVSSIGLCRYGGRIDCCWGWARQSWGQC |
| NOV3e | -------------------TGSEFDG-RWPRQIVSSIGLCRYGGRIDCCWGWARQSWGQC |
| NOV3f | TGSMDFLLALVLVSSLYLQAAAEFDGSRWPRQIVSSIGLCRYGGRIDCCWGWARQSWGQC |
| NOV3g | --------------------------------------------------------TGSC |
| NOV3h | --------------------------------------------------------TGSC |
| NOV3a | QP-----------------VCQPRCKHGECIGPNKCKCHPGYAGKTCNQDLNECGLKPRP |
| NOV3b | QPFYVLRQRTARIRCQLKAVCQPRCKHGECIGPNKCKCHPGYAGKTCNQDLNECGLKPRP |
| NOV3c | QP-----------------VCQPRCKHGECTGPNKCKCHPGYAGKTCNQDLNECGLKPRP |
| NOV3d | QPFYVLRQRIAPTRCQLKAVCQPRCKHGECIGPNKCKCHPGYAGKTCNQDLNECGLKPRP |
| NOV3e | QP-----------------VCQPRCKHGECIGPNKCKCHPGYAGKTCNQDLNECGLKPRP |
| NOV3f | QP-----------------VCQPRCKHGECIGPNXCKCHPGYAGKTCNQDLNECGLKPRP |
| NOV3g | QP-----------------VCQPRCKHGECIGPNKCKCHPGYAGKTCNQDLNECGLKPRP |
| NOV3h | QP-----------------VCQPRCKHGECIGPNKCKCHPGYAGKTCNQDLNECGLKPRP |
| NOV3a | CKHRCMNTYGSYKCYCLNGYMLMPDGSCSSALTCSMANCQYGCDVVKGQIRCQCPSPGLQ |
| NOV3b | CKHRCMNTYGSYKCYCLNGYMLMPDGSCSSALTCSMANCQYGCDVVKGQIRCQCPSPGLQ |
| NOV3c | CKHRCMNTYGSYKCYCLNGYMLMPDGSCSSALTCSMANCQYGCDVVKGQIRCQCPSPGLH |
| NOV3d | CKHRCMNTYGSYKCYCLNGYMLMPDGSCSSALTCSMANCQYGCDVVKGQIRCQCPSPGLH |
| NOV3e | CKHRCMNTYGSYKCYCLNGYMLMPDGSCSSALTCSMANCQYGCDVVKGQIRCQCPSPGLH |
| NOV3f | CKHRCMNTYGSYKCYCLNGYMLMPDGSCSSALTCSMANCQYGCDVVKGQIRCQCPSPGLH |
| NOV3g | CKHRCMNTYGSYKCYCLNGYMLMPDGSCSSALTCSMANCQYGCDVVKGQIRCQCPSPGLQ |
| NOV3h | CKHRCMNTYGSYKCYCLNGYMLMPDGSCSSALTCSMANCQYGCDVVKGQIRCQCPSPGLH |
| NOV3a | LAPDGRTCVDVDECATGRASCPRFRQCVNTFGSYICKCHKGFDLMYIGGKYQCHDTDECS |
| NOV3b | LAPDGRTCVDVDECATGRASCPRFRQCVNTFGSYICKCHKGFDLMYIGGKYQCHDIDECS |
| NOV3c | LAPDGRTCVDVDECATGRASCPRFRQCVNTFGSYICKCHKGFDLMYIGGKYQCHDIDECS |
| NOV3d | LAPDGRTCVDVDECATGRASCPRFRQCVNTFGSYICKCHKGFDLMYIGGKYQCHDIDECS |
| NOV3e | LAPDGRTCVDVDECATGPASCPRFRQCVNTFGSYICKCHKGFDLMYIGGKYQCHDIDECS |
| NOV3f | LAPDGRTCVDVDECATGPASCPRFRQCVNTFGSYICKCHKGFDLMYIGGKYQCHDIDECS |
| NOV3g | LAPDGRTCVDVDECATGHASCPRFRQCVNTFGSYICKCHKGFDLMYIGGKYQCHDIDECS |
| NOV3h | LAPDGRTCVDVDECATGHASCPRFRQCVNTFGSYICKCHKGFDLMYIGGKYQCHDIDECS |
| NOV3a | LGQYQCSSFARCYNIRGSYKCKCKEGYQGDGLTCVYIPKVMIEPSGPIHVPKGNGTILKG |
| NOV3b | LGQYQCSSFARCYNVRGSYKCKCKEGYQGDGLTCVYIPKVMIEPSGPIHVPKGNGTILKG |
| NOV3c | LGQYQCSSFARCYNVRGSYKCKCKEGYQGDGLTCVYIPKVMIEPSGPIHVPKGNGTILKG |
| NOV3d | LGQYQCSSFARCYNVRGSYKCKCKEGYQGDGLTCVYIPKVMIEPSGPIHVPKGNGTILKG |
| NOV3e | LGQYQCSSFARCYNVRGSYKCKCKEGYQGDGLTCVYIPKVMIEPSGPIHVPKGNGTILKG |
| NOV3f | LGQYQCSSFARCYNVRGSYKCKCKEGYQGDGLTCVYIPKVMIEPSGPIHVPKGNGTILKG |
| NOV3g | LGQYQCSSFARCYNVRGSYKCKCKEGYQGDGLTCVDG----------------------- |
| NOV3h | LGQYQCSSFARCYNVRGSYKCKCKEGYQGDGLTCVDG----------------------- |
| NOV3a | DTGNNNWIPDVGSTWWPPKTPYIPPIITNRPTSKPTTRPTPKPTPIPTPPPPPPLPTELR |
| NOV3b | DTGNNNWIPDVGSTWWPPKTPYIPPTITNRPTSKPTTRPTPKPTPIPTPPPPPPLPTELR |
| NOV3c | DTGNNNWIPDVGSTWWPPKTPYIPPIITNRPTSKPTTRPTPKPTPIPTPPPPPPLPTELR |
| NOV3d | DTGNNNWIPDVGSTWWPPKTPYIPPIITNRPTSKPTTRPTPKPTPIPTPPPPPPLPTELR |
| NOV3e | DTGNNNWIPDVGSTWWPPKTPYIPPIITNRPTSKPTTRPTPKPTPIPTPPPPPPLPTELR |
| NOV3f | DTGNNNWIPDVGSTWWPPKTPYIPPIITNRPTSKPTTRPTPKPTPIPTPPPPPPLPTELR |
| NOV3g | ------------------------------------------------------------ |
| NOV3h | ------------------------------------------------------------ |
| NOV3a | TPLPPTTPERPTTGLTTIAPAASTPPGGITVDNRVQTDPQKPRGDVFS------------ |
| NOV3b | TPLPPTTPERPTTGLTTIAPAASTPPGGITVDNRVQTDPQKPRGDVFTPRQPSNDLFEIF |
| NOV3c | TPLPPTTPERPTTGLTTIAPAASTPPGGITVDNRVQTDPQKPRGDVFS------------ |
| NOV3d | TPLPPTTPERPTTGLTTIAPAASTPPGGITVDNRVQTDPQKPRGDVFTPRQPSNDLFEIF |
| NOV3e | TPLPPTTPERPTTGLTTIAPAASTPPGGITVDNRVQTDPQKPRGDVFTPRQPSNDLFEIF |
| NOV3f | TPLPPTTPERPTTGLTTIAPAASTPPGGITVDNRVQTDPQKPRGDVFTPRQPSNDLFEIF |
| NOV3g | ------------------------------------------------------------ |
| NOV3h | ------------------------------------------------------------ |
| NOV3a | -----------------VLVHSCNFDHGLCGWIREKDNDLHWEPIPDPAGGQYLTVSAAK |
| NOV3b | EIERGVSADDEAKDDPGVLVHSCNFDHGLCGWIREKDNDLHWEPIPDPAGGQYLTVSAAK |
| NOV3c | -----------------VLVHSCNFDHGLCGWIREKDNDLHWEPIRDPAGGQYLTVSAAK |
| NOV3d | EIERGVSADDEAKDDPGVLVHSCNFDHGLCGWIREKDNDLHWEPIRDPAGGQYLTVSAAK |
| NOV3e | EIERGVSADDEAKDDPGVLVHSCNFDHGLCGWIREKDNDLHWEPIRDPAGGQYLTVSAAK |
| NOV3f | EIERGVSADDEAKDDPGVLVHSCNFDHGLCGWIREKDNDLHWEPIRDPAGGQYLTVSAAK |
| NOV3g | ------------------------------------------------------------ |
| NOV3h | ------------------------------------------------------------ |
| NOV3a | APGGKAARLVLPLGRLMHSGDLCLSFRHKVTGLHSGTLQVFVRKHGAHGAALWGRNGGHG |
| NOV3b | APGGKAARLVLPLGRLMHSGDLCLSFRHKVTGLHSGTLQVFVRKHGAHGAALWGRNGGHG |
| NOV3c | APGGKAARLVLPLGRLMHSGDLCLSFRHKVTGLHSGTLQVFVRKHGAHGAALWGRNGGHG |
| NOV3d | APGGKAARLVLPLGRLMHSGDLCLSFRHKVTGLHSGTLQVFVRKHGAHGAALWGRNGGHG |
| NOV3e | APGGKAARLVLPLGRLMHSGDLCLSFRHKVTGLHSGTLQVFVRKHGAHGAALWGRNGGHG |
| NOV3f | APGGKAARLVLPLGRLMHSGDLCLSFRHKVTGLHSGTLQVFVRKHGAHGAALWGRNGGHG |
| NOV3g | ------------------------------------------------------------ |
| NOV3h | ------------------------------------------------------------ |
| NOV3a | WRQTQITLRGADIKSVVFKGEKRRGHTGEIGLDDVSLKKGHCSEER--- |
| NOV3b | WRQTQITLRGADIKSVVFKGEKRRGHTGEIGLDDVSLKKGHCSEERVDG |
| NOV3c | WRQTQITLRGADIKSVVFKGEKRRGHTGEIGLDDVSLKKGHCSEERVDG |
| NOV3d | WRQTQITLRGADIKSVVFKGEKRRGHTGEIGLDDVSLKKGHCSEERVDG |
| NOV3e | WRQTQITLRGADIKSVVFKGEKRRGHTGEIGLDDVSLKKGHCSEERVDG |
| NOV3f | WRQTQITLRGADIKSVVFKGEKRRGHTGEIGLDDVSLKKGHCSEERVDG |
| NOV3g | ------------------------------------------------- |
| NOV3h | ------------------------------------------------- |
| NOV3a | (SEQ ID NO: 6) |
| NOV3b | (SEQ ID NO: 8) |
| NOV3c | (SEQ ID NO: 10) |
| NOV3d | (SEQ ID NO: 12) |
| NOV3e | (SEQ ID NO: 14) |
| NOV3f | (SEQ ID NO: 16) |
| NOV3g | (SEQ ID NO: 18) |
| NOV3h | (SEQ ID NO: 20) |

Further analysis of the NOV3a protein yielded the following properties shown in Table 3C.

TABLE 3C
Protein Sequence Properties NOV3a

| | |
|---|---|
| SignalP analysis: | Cleavage site between residues 20 and 21 |
| PSORT II analysis: | |

| PSG: | a new signal peptide prediction method | |
|---|---|---|
| | N-region: | length 2; pos. chg 0; neg. chg 1 |
| | H-region: | length 17; peak value 0.00 |
| | PSG score: | −4.40 |

| | |
|---|---|
| GvH: | von Heijne's method for signal seq. recognition |
| | GvH score (threshold: −2.1): −0.34 |
| | possible cleavage site: between 17 and 18 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 1 | | |
| | Number of TMS(s) for threshold 0.5: 1 | | |
| | INTEGRAL | Likelihood = | Transmembrane 3-19 |
| | | −4.19 | |

| | |
|---|---|
| PERIPHERAL | Likelihood = 5.67 (at 439) |

| | |
|---|---|
| | ALOM score: −4.19 (number of TMSs: 1) |
| MTOP: | Prediction of membrane topology (Hartmann et al.) |
| | Center position for calculation: 10 |
| | Charge difference: 0.0 C(0.0)-N(0.0) |
| | N >= C: N-terminal side will be inside |

>>> membrane topology: type 2 (cytoplasmic tail 1 to 3)

| MITDISC: | discrimination of mitochondrial targeting seq | | | |
|---|---|---|---|---|
| | R content: | 0 | Hyd Moment(75): | 4.41 |
| | Hyd Moment(95): | 7.23 | G content: | 0 |
| | D/E content: | 2 | S/T content: | 2 |

| | |
|---|---|
| | Score: −6.55 |
| Gavel: | prediction of cleavage sites for mitochondrial preseq |
| | cleavage site motif not found |

NUCDISC: discrimination of nuclear localization signals

| | |
|---|---|
| | pat4: none |
| | pat7: none |
| | bipartite: none |
| | content of basic residues: 11.9% |
| | NLS Score: −0.47 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 89 |
| COIL: | Lupas's algorithm to detect coiled-coil regions |
| | total: 0 residues |

Final Results (k = 9/23):

```
43.5%: nuclear
26.1%: mitochondrial
  8.7%: cytoplasmic
  4.3%: plasma membrane
  4.3%: vesicles of secretory system
  4.3%: extracellular, including cell wall
  4.3%: endoplasmic reticulum
  4.3%: peroxisomal
```

>> prediction for CG50253-01 is nuc (k = 23)

A search of the NOV3a protein against the Geneseq database, a proprietary database that contains sequences published in patents and patent publication, yielded several homologous proteins shown in Table 3D.

TABLE 3D
Geneseq Results for NOV3a

| | | NOV3a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABG70290 | Human novel polypeptide #6 - Homo | 1 . . . 536 | 536/536 (100%) | 0.0 |
| | sapiens, 536 aa. [WO200257452-A2, | 1 . . . 536 | 536/536 (100%) | |
| | 25-JUL-2002] | | | |
| ABG69659 | Human secreted protein SCEP-39 - | 1 . . . 536 | 534/566 (94%) | 0.0 |
| | Homo sapiens, 566 aa. | 1 . . . 566 | 535/566 (94%) | |
| | [WO200248337-A2, 20-JUN-2002] | | | |
| AAB70547 | Human PRO17 protein sequence SEQ | 1 . . . 536 | 532/582 (91%) | 0.0 |
| | ID NO: 34 - Homo sapiens, 582 aa. | 1 . . . 582 | 533/582 (91%) | |
| | [WO200110902-A2, 15-FEB-2001] | | | |
| AAB70549 | Clone 16467945.0.85-S261.D protein | 20 . . . 536 | 512/546 (93%) | 0.0 |
| | sequence SEQ ID NO: 82 - Homo | 1 . . . 546 | 514/546 (93%) | |
| | sapiens, 546 aa. [WO200110902-A2, | | | |
| | 15-FEB-2001] | | | |
| ABU58425 | Human PRO polypeptide #26 - Homo | 1 . . . 505 | 503/505 (99%) | 0.0 |
| | sapiens, 509 aa. [US2003027272-A1, | 1 . . . 505 | 504/505 (99%) | |
| | 06-FEB-2003] | | | |

In a BLAST search of public sequence databases, the NOV3a protein was found to have homology to the proteins shown in the BLASTP data in Table 3E.

TABLE 3E
Public BLASTP Results for NOV3a

| | | NOV3a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAC33425 | Sequence 33 from Patent | 1 . . . 536 | 532/582 (91%) | 0.0 |
| | WO0110902 - Homo sapiens | 1 . . . 582 | 533/582 (91%) | |
| | (Human), 582 aa. | | | |
| Q91V88 | POEM (NEPHRONECTIN short | 1 . . . 532 | 470/562 (83%) | 0.0 |
| | isoform) - Mus musculus (Mouse), | 1 . . . 561 | 497/562 (87%) | |
| | 561 aa. | | | |
| Q91ZD3 | Nephronectin long isoform - Mus | 1 . . . 532 | 470/579 (81%) | 0.0 |
| | musculus (Mouse), 578 aa. | 1 . . . 578 | 497/579 (85%) | |
| Q91XL5 | Nephronectin - Mus musculus | 1 . . . 532 | 470/593 (79%) | 0.0 |
| | (Mouse), 592 aa. | 1 . . . 592 | 497/593 (83%) | |
| Q923T5 | Nephronectin - Mus musculus | 1 . . . 532 | 470/610 (77%) | 0.0 |
| | (Mouse), 609 aa. | 1 . . . 609 | 497/610 (81%) | |

PFam analysis predicts that the NOV3a protein contains the domains shown in the Table 3F.

TABLE 3F
Domain Analysis of NOV3a

| | | | |
|---|---|---|---|
| | | Identities/ | |
| | | Similarities for | |
| Pfam | NOV3a | the Matched | Expect |
| Domain | Match Region | Region | Value |

| | | | | |
|---|---|---|---|---|
| C_tripleX | 54 . . . 71 | 7/18 | (39%) | 0.24 |
| | | 15/18 | (83%) | |
| EGF | 60 . . . 86 | 13/47 | (28%) | 0.13 |
| | | 23/47 | (49%) | |
| EGF | 93 . . . 127 | 15/47 | (32%) | 0.51 |
| | | 27/47 | (57%) | |
| EGF | 133 . . . 167 | 10/47 | (21%) | 0.035 |
| | | 24/47 | (51%) | |
| EGF | 218 . . . 253 | 12/47 | (26%) | 0.00011 |
| | | 28/47 | (60%) | |
| MAM | 393 . . . 534 | 48/183 | (26%) | 8.6e−27 |
| | | 98/183 | (54%) | |

### Example 4

The NOV4 clone was analyzed, and the nucleotide and encoded polypeptide sequences are shown in Table 4A.

TABLE 4A
NOV4 Sequence Analysis

| | | |
|---|---|---|
| NOV4a, CG50377-04 | SEQ ID NO: 31 | 10655 bp |
| DNA Sequence | ORF Start: at 1 | ORF Stop: TAG at 10639 |

GGCCAGAACTGCACGTTCCAACTGCACGGTCCCAATGGGACAGTTGAGAGCCCAGGGTTCCCATATGG
CTACCCCAATTACGCCAACTGCACGTGGACCATCACCGCGGAAGAGCAGCACAGAATCCAGCTTGTGT
TCCAGTCCTTTGCCCTGGAAGAGGACTTTGATGTCCTGTCGGTGTTTGATGGTCCACCCCAGCCAGAG
AATCTGCGTACGAGGCTCACAGGCTTTCAGCTGCCAGCCACCATTGTTAGTGCAGCCACCACCCTCTC
TCTGCGCCTCATCAGCGACTATGCAGTCAGTGCCCAAGGCTTCCACGCCACCTATGAAGTTCTCCCCA
GCCACACATGTGGGAACCCAGGGAGGCTGCCCAATGGCATCCAGCAGGGTTCAACCTTCAACCTCGGT
GACAAGGTCCGCTACAGCTGCAACCTTGGCTTCTTCCTGGAGGGCCACGCCGTGCTCACCTGCCACGC
TGGCTCTGAGAACAGCGCCACGTGGGACTTCCCCCTGCCTTCCTGCAGAGCTGATGATGCCTGTGGTG
GGACCCTGCGGGGCCAGAGTGGCATCATCTCCAGCCCCCACTTCCCCTCGGAGTACCATAACAATGCC
GACTGCACATGGACCATCCTGGCTGAGCTGGGGGACACCATCGCCCTGGTGTTTATTGACTTCCAGCT
GGAGGATGGTTACGACTTTCTGGAAGTCACTGGGACAGAAGGCTCCTCCCTCTGGTTCACCGGAGCCA
GCCTCCCAGCCCCCGTTATCAGCAGCAAGAACTGGCTGCGACTGCACTTCACATCGGATGGCAACCAC
CGGCAGCGCGGATTCAGTGCCCAATACCAAGTCAAGAAGCAAATTGAGTTGAAGTCTCGAGGTGTGAA
GCTGATGCCCAGCAAAGACAACAGCCAGAAGACGTCTGTGGTAACTCAGGTTGGTGTGTCCCAAGGAC
ATAATATGTGTCCAGACCCTGGCATACCCGAAAGGGGCAAAAGACTAGGCTCGGATTTCAGGTTAGGA
TCCAGCGTCCAGTTCACCTGCAACGAGGGCTATGACCTGCAAGGGTCCAAGCGGATCACCTGTATGAA
AGTGAGCGACATGTTTGCGGCCTGGAGCGACCACAGGCCAGTCTGCCGAGCCCGCATGTGTGATGCCC
ACCTTCGAGGCCCCTCGGGCATCATCACCTCCCCCAATTTCCCCATTCAGTATGACAACAATGCACAC
TGTGTGTGGATCATCACAGCACTCAACCCCTCCAAGGTAATCAAGCTCGCCTTTGAGGAGTTTGATTT
GGAGAGGGGCTATGACACCCTGACGGTCGGTGATGGTGGTCAGGATGGGGACCAGAAGACAGTTCTCT
ACATGCTGACAGGTACATCGGTCCCGGATCTCATTGTCAGCACCAATCATCAAATGTGGCTCCTCTTC
CAGACTGATGGCAGTGGCAGTTCCCTGGGATTCAAGGCTTCTTATGAAGAGATCGAGCAGGGCAGTTG
CGGTGACCCTGGCATACCTGCATATGGCCGGAGGGAAGGCTCCCGGTTTCGCCACGGTGACACACTCA
AGTTTGAGTGCCAGCCCGCCTTTGAGCTGGTGGGACAGAAGGCAATCACATGCCAAAAGAATAACCAA
TGGTCGGCTAAGAAGCCAGGCTGCGTGTGTTCCTGCTTCTTCAACTTCACCAGCCCGTCTGGGGTTGT
CCTGTCTCCCAACTACCCAGAGGACTATGGCAACCACCTCCACTGTGTCTGGCTCATCCTGGCCAGGC
CTGAGAGCCGCATCCACCTGGCCTTCAACGACATTGACGTGGAGCCTCAGTTTGATTTCCTGGTCATC
AAGGATGGGGCCACCGCCGAGGCGCCCGTCCTGGGCACCTTCTCAGGAAACCAGCTTCCCTCCTCCAT
CACAAGCAGTGGCCACGTGGCCCGTCTCGAGTTCCAGACTGACCACTCCACAGGGAAGAGGGGCTTCA
ACATCACTTTTACCAGTGAGTCCTCCAACGAGTGCCCGGATCCTGGCGTTCCAGTAAATGGCAAACGG
TTTGGGGACAGCCTCCAGCTGGGCAGCTCCATCTCCTTCCTCTGTGATGAAGGCTTCCTTGGGACTCA
GGGCTCAGAGACCATCACCTGCGTCCTGAAGGAGGGCAGCGTGGTCTGGAACAGCGCTGTGCTGCGGT
GTGAAGCTCCCTGTGGTGGTCACCTGACTTCGCCCAGCGGCACCATCCTCTCTCCGGGCTGGCCTGGC
TTCTACAAGGATGCCTTGAGCTGTGCCTGGGTGATTGAGGCCCAGCCAGGCTACCCCATCAAAATCAC
CTTCGACAGGTTCAAAACCGAGGTCAACTATGACACCCTGGAAGTACGCGATGGGCGGACTTACTCAG
CGCCCTTGATCGGGGTTTACCACGGGACCCAGGTTCCCCAGTTCCTCATCAGCACCAGCAACTACCTC
TACCTCCTCTTCTCTACCGACAAGAGTCACTCGGACATCGGCTTCCAGCTCCGCTATGAGGCTATAAC
ACTGCAGTCAGACCACTGTCTGGATCCAGGAATCCCAGTAAATGGACAGCGTCATGGGAATGACTTCT
ACGTGGGCGCGCTGGTGACCTTCAGCTGTGACTCGGGCTACACATTAAGTGACGGGGAGCCTCTGGAG
TGTGAGCCCAACTTCCAGTGGAGCCGGGCCCTGCCCAGTTGTGAAGCTCTCTGTGGTGGCTTCATTCA
AGGCTCCAGTGGGACCATCTTGTCGCCAGGTTTCCCTGACTTCTACCCCAACAACTTGAACTGCACCT
GGATTATCGAAACATCTCATGGCAAGGGTGTGTTCTTCACTTTCCACACCTTCCACCTGGAAAGTGGC
CATGACTACCTCCTCATCACTGAGAACGGCAGCTTCACCCAGCCCCTGAGGCAGCTAACTGGATCTCG
GCTGCCAGCTCCCATCAGCGCTGGGCTCTATGGCAACTTCACTGCCCAGGTCCGCTTCATCTCTGATT
TCTCCATGTCATATGAAGGATTCAACATCACCTTCTCAGAGTACGACTTGGAGCCCTGTGAGGAGCCC
GAGGTCCCAGCCTACAGCATCCGGAAGGGCTTGCAGTTTGGCGTGGGCGACACCTTGACCTTCTCCTG
CTTCCCCGGGTACCGTCTGGAGGGCACCGCCCGCATCACGTGCCTGGGGGGCAGACGGCGCCTGTGGA
GCTCGCCTCTGCCAAGGTGTGTTGCTGAGTGTGGGAATTCAGTCACAGGCACTCAGGGTACTTTGCTG
TCCCCCAACTTTCCTGTGAACTACAATAACAATCATGAATGCATCTACTCCATCCAGACCCAGCCAGG
GAAGGGAATTCAGCTGAAAGCCAGGGCATTCGAACTCTCCGAAGGAGATGTCCTCAAGGTTTATGATG
GCAACAACAACTCCGCCCGTTTGCTGGGAGTTTTTAGCCATTCTGAGATGATGGGGGTGACTTTGAAC
AGCACATCCAGCAGTCTGTGGCTTGATTTCATCACTGATGCTGAAAACACCAGCAAGGGCTTTGAACT
GCACTTTTCCAGTTTTGAACTCATCAAATGTGAGGACCCAGGAACCCCCAAGTTTGGCTACAAGGTTC
ATGATGAAGGTCATTTTGCAGGGAGCTCCGTGTCCTTCAGCTGTGACCCTGGATACAGCCTGCGGGGT
AGTGAGGAGCTGCTGTGTCTGAGTGGAGAGCGCCGGACCTGGGACCGGCCTCTGCCCACCTGTGTCGC
CGAGTGTGGAGGGACAGTGAGAGGAGAGGTGTCGGGGCAGGTGCTGTCACCCGGGTATCCAGCTCCCT
ATGAACACAATCTCAACTGCATCTGGACCATCGAAGCAGAGGCCGGCTGCACCATTGGGCTACACTTC
CTGGTGTTTGACACAGAGGAGGTTCACGACGTGCTGCGCATCTGGGATGGGCCTGTGGAGAGCGGGGT
TCTGCTGAAGGAGCTGAGTGGCCCGGCCCTGCCCAAGGACCTGCATAGCACCTTCAACTCGGTCGTCC
TGCAGTTCAGCACTGACTTCTTCACCAGCAAGCAGGGCTTTGCCATTCAATTTTCAGGTTCCACAGCA
ACGTCCTGCAATGACCCTGGGATCCCGCAGAATGGGAGTCGGAGTGGTGACAGTTGGGAAGCCGGCGA
CTCCACAGTGTTCCAGTGTGACCCTGGCTACGCGCTGCAGGGAAGTGCAGAGATCAGCTGTGTGAAGA
TCGAGAACAGGTTCTTCTGGCAGCCCAGCCCGCCAACATGCATCGCTCCCTGCGGGGGAGACCTGACA
GGACCATCTGGAGTCATCCTCTCACCAAATTACCCAGAACCCTACCCGCCAGGCAAGGAGTGTGACTG
GAAAGTGACCGTCTCACCAGACTACGTCATCGCCCTGGTATTGTTCCCCAGCTTTAACCTGGAGCCTG
GCTATGACTTCCTCCATATCTACGACGGACGGGACTCTCTCAGCCCTCTCATAGGAAGCTTCTATGGC
TCCCAGCTCCCAGGCCGCATTGAAAGCAGCAGCAACAGCCTCTTCCTCGCCTTCCGCAGCGATGCATC
TGTGAGCAATGCTGGCTTCGTCATTGACTTCCCAGAAAACCCGCGGGAGTCATGTTTTGATCCTGGTT
CCATCAAGAACGGCACACGGGTGGGGTCCGACCTGAAGCTGGGCTCCTCCGTCACCTACTACTGCCAC
GGGGGCTACGAAGTTGAGGGCACCTCGACCCTGAGCTGCATCCTGGGGCCTGATGGGAAGCCCGTGTG
GAACAATCCCCGGCCAGTCTGCACAGCCCCCTGTGGGGGACAGTATGTGGGTTCGGACGGAGTGGTCT
TGTCCCCCAACTACCCCCAGAACTACACCAGTGGACAGATCTGCTTGTATTTTGTTACTGTGCCCAAG
GACTATGGTGTGGTGTTTGGCCAGTTCGCCTTCTTTCACACGGCCCTCAACGACGTGGTGGAGGTTCA
CGACGGCCACAGCCAGCACTCGCGGCTCCTCAGCTCCCTCTCGGGCTCCCATACAGGTGAATCACTGC
CCTTGGCCACCTCCAATCAAGTTCTCATTAAGTTCAGCGCCAAAGGCCTCGCACCAGCCAGAGGCTTC
CACTTTGTCTACCAAGCGGTTCCTCGAACCAGCGCCACGCAGTGCAGCTCTGTGCCGGAACCCCGCTA
TGGCAAGAGGCTGGGCAGTGACTTCTCGGTGGGGGCCATCGTCCGCTTCGAATGCAACTCCGGCTATG
CCCTGCAGGGGTCGCCAGAGATCGAGTGCCTCCCTGTGCCTGGGGCCTTGGCCCAATGGAATGTCTCA
GCGCCCACGTGTGTGGTGCCGTGTGGAGGCAACCTCACAGAGCGCAGGGGCACCATCCTGTCCCCTGG
CTTCCCAGAGCCGTACCTCAACAGCCTCAACTGTGTGTGGAAGATCGTGGTCCCCGAAGGCGCTGGCA
TCCAGATCCAAGTTGTCAGTTTTGTGACAGAGCAGAACTGGGACTCGCTGGAAGTATTTGATGGTGCA
GATAACACTGTAACCATGCTGGGGAGTTTCTCAGGAACAACCGTGCCTGCCCTTCTGAACAGCACCTC
CAACCAGCTCTACCTTCATTTCTACTCAGATATCAGCGTATCTGCAGCTGGCTTCCACTTGGAGTACA
AAACGGTGGGCCTGAGCAGTTGTCCGGAACCTGCTGTGCCCAGTAACGGGGTGAAGACTGGCGAGCGC
TACTTGGTGAATGATGTGGTGTCTTTCCAGTGTGAGCCGGGATATGCCCTCCAGGGCCACGCCCACAT
CTCCTGCATGCCCGGAACAGTGCGGCGATGGAACTACCCTCCTCCACTCTGTATTGCACAGTGTGGGG
GAACAGTGGAGGAGATGGAGGGGGTGATCCTGAGCCCCGGCTTCCCAGGCAACTACCCCAGTAACATG
GACTGCTCCTGGAAAATAGCACTGCCCGTGGGCTTTGGAGCTCACATCCAGTTCCTGAACTTCTCCAC
CGAGCCCAACCACGACTACATAGAAATCCGGAATGGCCCCTATGAGACCAGCCGCATGATGGGAAGAT
TCAGTGGAAGCGAGCTTCCAAGCTCCCTCCTCTCCACGTCCCACGAGACCACCGTGTATTTCCACAGC
GACCACTCCCAGAATCGGCCAGGATTCAAGCTGGAGTATCAGGCCTATGAACTTCAAGAGTGCCCAGA
CCCAGAGCCCTTTGCCAATGGCATTGTGAGGGGAGCTGGCTACAACGTGGGACAATCAGTGACCTTCG
AGTGCCTCCCGGGGTATCAATTGACTGGCCACCCTGTCCTCACGTGTCAACATGGCACCAACCGGAAC
TGGGACCACCCCCTGCCCAAGTGTGAAGTCCCTTGTGGCGGGAACATCACTTCTTCCAACGGCACTGT
GTACTCCCCGGGGTTCCCTAGCCCGTACTCCAGCTCCCAGGACTGTGTCTGGCTGATCACCGTGCCCA
TTGGCCATGGCGTCCGCCTCAACCTCAGCCTGCTGCAGACAGAGCCCTCTGGAGATTTCATCACCATC
TGGGATGGGCCACAGCAAACAGCACCACGGCTCGGCGTCTTCACCCGGAGCATGGCCAAGAAAACAGT
GCAGAGTTCATCCAACCAGGTCCTGCTCAAGTTCCACCGTGATGCAGCCACAGGGGGGATCTTCGCCA
TAGCTTTCTCCGCTTATCCACTCACCAAATGCCCTCCTCCCACCATCCTCCCCAACGCCGAAGTCGTC
ACAGAGAATGAAGAATTCAATATAGGTGACATCGTACGCTACAGATGCCTCCCTGGCTTTACCTTAGT
GGGGAATGAAATTCTGACCTGCAAACTTGGAACCTACCTGCAGTTTGAAGGACCACCCCCGATATGTG
AAGTGCACTGTCCAACAAATGAGCTTCTGACAGACTCCACAGGCGTGATCCTGAGCCAGAGCTACCCT
GGAAGCTATCCCCAGTTCCAGACCTGCTCTTGGCTGGTGAGAGTGGAGCCCGACTATAACATCTCCCT
CACAGTGGAGTACTTCCTCAGCGAGAAGCAATATGATGAGTTTGAGATTTTTGATGGTCCATCAGGAC
AGAGTCCTCTGCTGAAAGCCCTCAGTGGGAATTACTCAGCTCCCCTGATTGTCACCAGCTCAAGCAAC
TCTGTGTACCTGCGTTGGTCATCTGATCACGCCTACAATCGGAAGGGCTTCAAGATCCGCTATTCAGC
CCCTTACTGCAGCCTGCCCAGGGCTCCACTCCATGGCTTCATCCTAGGCCAGACCAGCACCCAGCCCG
GGGGCTCCATCCACTTTGGCTGCAACGCCGGCTACCGCCTGGTGGGACACAGCATGGCCATCTGTACC
CGGCACCCCCAGGGCTACCACCTGTGGAGCGAAGCCATCCCTCTCTGTCAAGCTCTTTCCTGTGGGCT
TCCTGAGGCCCCCAAGAATGGAATGGTGTTTGGCAAGGAGTACACAGTGGGAACCAAGGCCATGTACA
GCTGCAGTGAAGGCTACCACCTCCAGGCAGGCGCTGAGGCCACTGCAGAGTGTCTGGACACAGGCCTA
TGGAGCAACCGCAATGTCCCACCACAGTGTGTCCCTGTGACTTGTCCTGATGTCAGTAGCATCAGCGT
GGAGCATGGCCGATGGAGGCTTATCTTTGAGACACAGTATCAGTTCCAGGCCCAGCTGATGCTCATCT
GTGACCCTGGCTACTACTATACTGGCCAAAGGGTCATCCGCTGTCAGGCCAATGGCAAATGGAGCCTC
GGGGACTCTACGCCCACCTGCCGAATCATCTCCTGTGGAGAGCTCCCGATTCCCCCCAATGGCCACCG
CATCGGAACACTGTCTGTCTACGGGGCAACAGCCATCTTCTCCTGCAATTCCGGATACACACTGGTGG
GCTCCAGGGTGCGTGAGTGCATGGCCAATGGGCTCTGGAGTGGCTCTGAAGTCCGCTGCCTTGCTGGA
CACTGTGGGACTCCTGAGCCCATTGTCAACGGACACATCAATGGGGAGAACTACAGCTACCGGGGCAG
TGTGGTGTACCAATGCAATGCTGGCTTCCGCCTGATCGGCATGTCTGTGCGCATCTGCCAGCAGGATC
ATCACTGGTCGGGCAAGACCCCTTTCTGTGTGCCAATTACCTGTGGACACCCAGGCAACCCTGTCAAC
GGCCTCACTCAGGGTAACCAGTTTAACCTCAACGATGTGGTCAAGTTTGTTTGCAACCCTGGGTATAT
GGCTGAGGGGGCTGCTAGGTCCCAATGCCTGGCCAGCGGGCAATGGAGTGACATGCTGCCCACCTGCA
GAATCATCAACTGTACAGATCCTGGACACCAAGAAAATAGTGTTCGTCAGGTCCACGCCAGCGGCCCG
CACAGGTTCAGCTTCGGCACCACTGTGTCTTACCGGTGCAACCACGGCTTCTACCTCCTGGGCACCCC
AGTGCTCAGCTGCCAGGGAGATGGCACATGGGACCGTCCCCGCCCCCAGTGTCTCCTGGTGTCCTGTG
GCCATCCGGGCTCCCCGCCTCACTCCCAGATGTCTGGAGACAGTTATACTGTGGGAGCAGTGGTGCGG
TACAGCTGCATCGGCAAGCGTACTCTGGTGGGAAACAGCACCCGCATGTGTGGGCTGGATGGACACTG
GACTGGCTCCCTCCCTCACTGCTCAGGAACCAGCGTGGGAGTTTGCGGTGACCCTGGGATCCCGGCTC
ATGGCATCCGTTTGGGGGACAGCTTTGATCCAGGCACTGTGATGCGCTTCAGCTGTGAAGCTGGCCAC
GTGCTCCGGGGATCGTCAGAGCGCACCTGTCAAGCCAATGGCTCGTGGAGCGGCTCGCAGCCTGAGTG
TGGAGTGATCTCTTGTGGGAACCCTGGGACTCCAAGTAATGCCCGAGTTGTGTTCAGTGATGGCCTGG
TTTTCTCCAGCTCTATCGTCTATGAGTGCCGGGAAGGATACTACGCCACAGGCCTGCTCAGCCGTCAC
TGCTCGGTCAATGGTACCTGGACAGGCAGTGACCCTGAGTGCCTCGTCATAAACTGTGGTGACCCTGG
GATTCCAGCCAATGGCCTTCGGCTGGGCAATGACTTCAGGTACAACAAAACTGTGACATATCAGTGTG
TCCCTGGCTATATGATGGAGTCACATAGAGTATCTGTGCTGAGCTGCACCAAGGACCGGACATGGAAT
GGAACCAAGCCCGTCTGCAAAGCTCTCATGTGCAAGCCACCTCCGCTCATCCCCAATGGGAAGGTGGT
GGGGTCTGACTTCATGTGGGGCTCAAGTGTGACTTATGCCTGCCTGGAGGGGTACCAGCTCTCCCTGC
CCGCGGTGTTCACCTGTGAGGGAAATGGGTCCTGGACCGGAGAGCTGCCTCAGTGTTTCCCTGTGTTC
TGCGGGGATCCTGGTGTCCCGTCCCGTGGGAGGAGAGAGGACCGAGGCTTCTCCTACAGGTCATCTGT
CTCCTTCTCCTGCCATCCCCCTCTGGTGCTGGTGGGCTCTCCACGCAGGTTTTGCCAGTCAGATGGGA
CATGGAGTGGCACCCAGCCCAGCTGCATAGATCCGACCCTGACCACGTGTGCGGACCCTGGTGTGCCA
CAGTTTGGGATACAGAACAATTCTCAGGGCTACCAGGTTGGAAGCACAGTCCTCTTCCGTTGTCAAAA
AGGCTACCTGCTTCAGGGCTCCACCACCAGGACCTGCCTCCCAAACCTGACCTGGAGTGGAACCCCAC
CTGACTGTGTCCCCCACCACTGCAGGCAGCCAGAGACGCCAACGCATGCCAACGTCGGGGCCCTGGAT
TTGCCCTCCATGGGCTACACGCTCATCTACTCCTGCCAGGAGGGCTTCTCCCTCAAGGGTGGCTCCGA
GCACCGCACCTGCAAGGCGGATGGCAGCTGGACAGGCAAGCCGCCCATCTGCCTGGAGGTCCGGCCCA
ATGGGAGACCCATCAACACTGCCCGGGAGCCACCGCTCACCCAAGCCTTGATTCCTGGGGATGTTTTT
GCCAAGAATTCCCTGTGGAAAGGGGCCTATGAATACCAGGGGAAGAAGCAGCCAGCCATGCTCAGAGT
GACTGGCTTCCAAGTTGCCAACAGCAAGGTCAATGCCACCATGATCGACCACAGTGGCGTGGAGCTGC
ACTTGGCTGGAACTTACAAGAAAGAAGATTTTCATCTCCTACTCCAGGTGTACCAGATTACAGGGCCT
GTGGAGATCTTTATGAATAAGTTCAAAGATGATCACTGGGCTTTAGATGGCCATGTCTCGTCAGAGTC
CTCCGGAGCCACCTTCATCTACCAAGGCTCTGTCAAGGGCCAAGGCTTTGGGCAGTTCGGCTTTCAAA
GACTGGACCTCAGGCTGCTGGAGTCAGACCCCGAGTCCATTGGCCGCCACTTTGCTTCCAACAGCAGC
TCAGTGGCAGCCGCGATCCTGGTGCCTTTCATCGCCCTCATTATTGCGGGCTTCGTGCTCTATCTCTA
CAAGCACAGGAGAAGACCCAAAGTTCCTTTCAATGGCTATGCTGGCCACGAGAACACCAATGTTCGGG
CCACATTTGAGAACCCAATGTACGACCGCAACATCCAGCCCACAGACATCATGGCCAGCGAGGCGGAG
TTCACAGTCAGCACAGTGTGCACAGCAGTATAGCCACCCGGCCTGGC

| | | | |
|---|---|---|---|
| NOV4a, CG50377-04 | SEQ ID NO: 32 | 3546 aa | MW at 385864.BkD |
| Protein Sequence | | | |

GQNCTFQLHGPNGTVESPGFPYGYPNYANCTWTITAEEQHRIQLVFQSFALEEDFDVLSVFDGPPQPE
NLRTRLTGFQLPATIVSAATTLSLRLISDYAVSAQGFHATYEVLPSHTCGNPGRLPNGIQQGSTFNLG
DKVRYSCNLGFFLEGHAVLTCHAGSENSATWDFPLPSCRADDACGGTLRGQSGIISSPHFPSEYHNNA
DCTWTILAELGDTIALVFIDFQLEDGYDFLEVTGTEGSSLWFTGASLPAPVISSKNWLRLHFTSDGNH
RQRGFSAQYQVKKQIELKSRGVKLMPSKDNSQKTSVVTQVGVSQGHNMCPDPGIPERGKRLGSDFRLG
SSVQFTCNEGYDLQGSKRITCMKVSDMFAAWSDHRPVCRAEMCDAHLRGPSGIITSPNFPIQYDNNAH
CVWIITALNPSKVIKLAFEEFDLERGYDTLTVGDGGQDGDQKTVLYMLTGTSVPDLIVSTNHQMWLLF
QTDGSGSSLGFKASYEEIEQGSCGDPGIPAYGRREGSRFRHGDTLKFECQPAFELVGQKAITCQKNNQ
WSAKKPGCVCSCFFNFTSPSGVVLSPNYPEDYGNHLHCVWLILARPESRIHLAFNDIDVEPQFDFLVI
KDGATAEAPVLGTFSGNQLPSSITSSGHVARLEFQTDHSTGKRGFNITFTSESSNECPDPGVPVNGKR
FGDSLQLGSSISFLCDEGFLGTQGSETITCVLKEGSVVWNSAVLRCEAPCGGHLTSPSGTILSPGWPG
FYKDALSCAWVIEAQPGYPIKITFDRFKTEVNYDTLEVRDGRTYSAPLIGVYHGTQVPQFLISTSNYL
YLLFSTDKSHSDIGFQLRYEAITLQSDHCLDPGIPVNGQRHGNDFYVGALVTFSCDSGYTLSDGEPLE
CEPNFQWSRALPSCEALCGGFIQGSSGTILSPGFPDFYPNNLNCTWIIETSHGKGVFFTFHTFHLESG
HDYLLITENGSFTQPLRQLTGSRLPAPISAGLYGNFTAQVRFISDFSMSYEGFNITFSEYDLEPCEEP
EVPAYSIRKGLQFGVGDTLTFSCFPGYRLEGTARITCLGGRRRLWSSPLPRCVAECGNSVTGTQGTLL
SPNFPVNYNNNHECIYSIQTQPGKGIQLKARAFELSEGDVLKVYDGNNNSARLLGVFSHSENMGVTLN
STSSSLWLDFITDAENTSKGFELHFSSFELIKCEDPGTPKFGYKVHDEGHFAGSSVSFSCDPGYSLRG
SEELLCLSGERRTWDRPLPTCVAECGGTVRGEVSGQVLSPGYPAPYEHNLNCIWTIEAEAGCTIGLHF
LVFDTEEVHDVLRIWDGPVESGVLLKELSGPALPKDLHSTFNSVVLQFSTDFFTSKQGFAIQFSGSTA
TSCNDPGIPQNGSRSGDSWEAGDSTVFQCDPGYALQGSAEISCVKIENRFFWQPSPPTCIAPCGGDLT
GPSGVILSPNYPEPYPPGKECDWKVTVSPDYVIALVLFPSFNLEPGYDFLHIYDGRDSLSPLIGSFYG
SQLPGRIESSSNSLFLAFRSDASVSNAGFVIDFPENPRESCFDPGSIKNGTRVGSDLKLGSSVTYYCH
GGYEVEGTSTLSCILGPDGKPVWNNPRPVCTAPCGGQYVGSDGVVLSPNYPQNYTSGQICLYFVTVPK
DYGVVFGQFAFFHTALNDVVEVHDGHSQHSRLLSSLSGSHTGESLPLATSNQVLIKFSAKGLAPARGF
HFVYQAVPRTSATQCSSVPEPRYGKRLGSDFSVGAIVRFECNSGYALQGSPEIECLPVPGALAQWNVS
APTCVVPCGGNLTERRGTILSPGFPEPYLNSLNCVWKIVVPEGAGIQIQVVSFVTEQNWDSLEVFDGA
DNTVTMLGSFSGTTVPALLNSTSNQLYLHFYSDISVSAAGFHLEYKTVGLSSCPEPAVPSNGVKTGER
YLVNDVVSFQCEPGYALQGHAHISCMPGTVRRWNYPPPLCIAQCGGTVEEMEGVILSPGFPGNYPSNM
DCSWKIALPVGFGAHIQFLNFSTEPNHDYIEIRNGPYETSRMMGRFSGSELPSSLLSTSHETTVYFHS
DHSQNRPGFKLEYQAYELQECPDPEPFANGIVRGAGYNVGQSVTFECLPGYQLTGHPVLTCQHGTNRN
WDHPLPKCEVPCGGNITSSNGTVYSPGFPSPYSSSQDCVWLITVPIGHGVRLNLSLLQTEPSGDFITI
WDGPQQTAPRLGVFTRSMAKKTVQSSSNQVLLKFHRDAATGGIFAIAFSAYPLTKCPPPTILPNAEVV
TENEEFNIGDIVRYRCLPGFTLVGNEILTCKLGTYLQFEGPPPICEVHCPTNELLTDSTGVILSQSYP
GSYPQFQTCSWLVRVEPDYNISLTVEYFLSEKQYDEFEIFDGPSGQSPLLKALSGNYSAPLIVTSSSN
SVYLRWSSDHAYNRKGFKIRYSAPYCSLPEAPLHGFILGQTSTQPGGSIHFGCNAGYRLVGHSMAICT
RHPQGYHLWSEAIPLCQALSCGLPEAPKNGMVFGKEYTVGTKAMYSCSEGYHLQAGAEATAECLDTGL
WSNRNVPPQCVPVTCPDVSSISVEHGRWRLIFETQYQFQAQLMLICDPGYYYTGQRVIRCQANGKWSL
GDSTPTCRIISCGELPIPPNGHRIGTLSVYGATAIFSCNSGYTLVGSRVRECMANGLWSGSEVRCLAG
HCGTPEPIVNGHINGENYSYRGSVVYQCNAGFRLIGMSVRICQQDHHWSGKTPFCVPITCGHPGNPVN
GLTQGNQFNLNDVVKFVCNPGYMAEGAARSQCLASGQWSDMLPTCRIINCTDPGHQENSVRQVHASGP
HRFSFGTTVSYRCNHGFYLLGTPVLSCQGDGTWDRPRPQCLLVSCGHPGSPPHSQMSGDSYTVGAVVR
YSCIGKRTLVGNSTRMCGLDGHWTGSLPHCSGTSVGVCGDPGIPAHGIRLGDSFDPGTVMRFSCEAGH
VLRGSSERTCQANGSWSGSQPECGVISCGNPGTPSNARVVFSDGLVFSSSIVYECREGYYATGLLSRH
CSVNGTWTGSDPECLVINCGDPGIPANGLRLGNDFRYNKTVTYQCVPGYMMESHRVSVLSCTKDRTWN
GTKPVCKALMCKPPPLIPNGKVVGSDFMWGSSVTYACLEGYQLSLPAVFTCEGNGSWTGELPQCFPVF
CGDPGVPSRGRREDRGFSYRSSVSFSCHPPLVLVGSPRRFCQSDGTWSGTQPSCIDPTLTTCADPGVP
QFGIQNNSQGYQVGSTVLFRCQKGYLLQGSTTRTCLPNLTWSGTPPDCVPHHCRQPETPTHANVGALD
LPSMGYTLIYSCQEGFSLKGGSEHRTCKADGSWTGKPPICLEVRPNGRPINTAREPPLTQALIPGDVF
AKNSLWKGAYEYQGKKQPAMLRVTGFQVANSKVNATMIDHSGVELHLAGTYKKEDFHLLLQVYQITGP
VEIFMNKFKDDHWALDGHVSSESSGATFIYQGSVKGQGFGQFGFQRLDLRLLESDPESIGRHFASNSS
SVAAAILVPFIALIIAGFVLYLYKHRRRPKVPFNGYAGHENTNVPATFENPMYDRNIQPTDIMASEAE
FTVSTVCTAV

| | | |
|---|---|---|
| NOV4b, CG50377-01 | SEQ ID NO: 33 | 10136 bp |
| DNA Sequence | ORF Start: ATG at 1 | ORF Stop: TGA at 9313 |

ATGGCGGGCGCCCCTCCCCCCGCCTTGCTGCTGCCTTGCAGTTTGATCTCAGACTGCTGTGCTAGCAA
TCAGCGACACTCCGTGGGCGTAGGACCCTCCGAGCTAGTCAAGAAGCAAATTGAGTTGAAGTCTCGAG
GTGTGAAGCTGATGCCCAGCAAAGACAACAGCCAGAAGACGTCTGTGTTAACTCAGGTTGGTGTGTCC
CAAGGACATAATATGTGTCCAGACCCTGGCATACCCGAAAGGGGCAAAAGACTAGGCTCGGATTTCAG
GTTAGGATCCAGCGTCCAGTTCACCTGCAACGAGGGCTATGACCTGCAAGGGTCCAAGCGGATCACCT
GTATGAAAGTGAGCGACATGTTTGCGGCCTGGAGCGACCACAGGCCAGTCTGCCGAGCCCGCATGTGT
GATGCCCACCTTCGAGGCCCCTCGGGCATCATCACCTCCCCCAATTTCCCCATTCAGTATGACAACAA
TGCACACTGTGTGTGGATCATCACAGCACTCAACCCCTCCAAGGTGATCAAGCTCGCCTTTGAGGAGT
TTGATTTGGAGAGGGGCTATGACACCCTGACGGTCGGTGATGGTGGTCAGGATGGGGACCAGAAGACA
GTTCTCTACATGTCTCAAAATGCCTGCAGTGACAGCCCTCACACCCCAGGCTCTCGCATCCCAGAGAG
CATGTCTGGGGACATCTGGAGGCAGAAATGGACTGTACTTGAGATCTGTCGTGACATTAGCAGTTCAG
ATGCAAGGTCAGGTTCAGTGAGGAAGTCTCCAAAGACTTCTAATGCTGTGGAACTTGTTGCTCCTGGG
ACAGAGATCGAGCAGGGCAGTTGCGGTGACCCTGGCATACCTGCATATGGCCGGAGGGAAGGCTCCCG
GTTTCACCACGGTGACACACTCAAGTTTGAGTGCCAGCCCGCCTTTGAGCTGGTGGGACAGAAGGCAA
TCACATGCCAAAAGAATAACCAATGGTCGGCTAAGAAGCCAGGCTGCGTGTTCTCCTGCTTCTTCAAC
TTCACCAGCCCGTCTGGGGTTGTCCTGTCTCCCAACTACCCAGAGGACTATGGCAACCACCTCCACTG
TGTCTGGCTCATCCTGGCCAGGCCTGAGAGCCGCATCCACCTGGCCTTCAACGACATTGACGTGGAGC
CTCAGTTTGATTTCCTGGTCATCAAGGATGGGGCCACCGCCGAGGCGCCCGTCCTGGGCACCTTCTCA
GGAAACCAGCTTCCCTCCTCCATCACAAGCAGTGGCCACGTGGCCCGTCTCGAGTTCCAGACTGACCA
CTCCACAGGGAAGAGGGGCTTCAACATCACTTTTACCACCTTCCGACACAACGAGTGCCCGGATCCTG
GCGTTCCAGTAAATGGCAAACGGTTTGGGGACAGCCTCCAGCTGGGCAGCTCCATCTCCTTCCTCTGT
GATGAAGGCTTCCTTGGGACTCAGGGCTCAGAGACCATCACCTGCGTCCTGAAGGAGGGCAGCGTGGT
CTGGAACAGCGCTGTGCTGCGGTGTGAAGCTCCCTGTGGTGGTCACCTGACTTCGCCCAGCGGCACCA
TCCTCTCTCCGGGCTGGCCTGGCTTCTACAAGGATGCCTTGAGCTGTGCCTGGGTGATTGAGGCCCAG
CCAGGCTACCCCATCAAAATCACCTTCGACAGATTCAAAACCGAGGTCAACTATGACACCCTGGAAGT
ACGCGATGGGCGGACTTACTCAGCGCCCTTGATCGGGGTTTACCACGGGACCCAGGTTCCCCAGTTCC
TCATCAGCACCAGCAACTACCTCTACCTCCTCTTCTCTACCGACAAGAGTCACTCGGACATCGGCTTC
CAGCTCCGCTATGAGACTATAACACTGCAGTCAGACCACTGTCTGGATCCAGGAATCCCAGTAAATGG
ACAGCGTCATGGGAATGACTTCTACGTGGGCGCGCTGGTGACCTTCAGCTGTGACTCGGGCTACACAT
TAAGTGACGGGGAGCCTCTGGAGTGTGAGCCCAACTTCCAGTGGAGCCGGGCCCTGCCCAGTTGTGAA
GCTCTCTGTGGTGGCTTCATTCAAGGCTCCAGTGGGACCATCTTGTCGCCAGGGTTCCCTGACTTCTA
CCCCAACAACTTGAACTGCACCTGGATTATCGAAACATCTCATGGCAAGGGTGTGTTCTTCACTTTCC
ACACCTTCCACCTGGAAAGTGGCCATGACTACCTCCTCATCACTGAGAACGGCAGCTTCACCCAGCCC
CTGAGGCAGCTAACTGGATCTCGGCTGCCAGCTCCCATCAGCGCTGGGCTCTATGGCAACTTCACTGC
CCAGGTCCGCTTCATCTCTGATTTCTCCATGTCATATGAAGGATTCAACATCACCTTCTCAGAGTACG
ACTTGGAGCCCTGTGAGGAGCCCGAGGTCCCAGCCTACAGCATCCGGAAGGGCTTGCAGTTTGGCGTG
GGCGACACCTTGACCTTCTCCTGCTTCCCCGGGTACCGTCTGGAGGGCACCGCCCGCATCACGTGCCT
GGGGGGCAGACGGCGCCTGTGGAGCTCGCCTCTGCCAAGGTGTGTTGCTGAGTGTGGGAATTCAGTCA
CAGGCACTCAGGGTACTTTGCTGTCCCCCAACTTTCCTGTGAACTACAATAACAATCATGAATGCATC
TACTCCATCCAGACCCAGCCAGGGAAGGGAATTCAGCTGAAAGCCAGGGCATTCGAACTCTCCGAAGG
AGATGTCCTCAAGGTTTATGATGGCAACAACAACTCCGCCCGTTTGCTGGGAGTTTTTAGCCATTCTG
AGATGATGGGGGTGACTTTGAACAGCACATCCAGCAGTCTGTGGCTTGATTTCATCACTGATGCTGAA
AACACCAGCAAGGGCTTTGAACTGCACTTTTCCAGCTTTGAACTCATCAAATGTGAGGACCCAGGAAC
CCCCAAGTTTGGCTACAAGGTTCATGATGAAGGTCATTTTGCAGGGAGCTCCGTGTCCTTCAGCTGTG
ACCCTGGATACAGCCTGCGGGGTAGTGAGGAGCTGCTGTGTCTGAGTGGAGAGCGCCGGACCTGGGAC
CGGCCTCTGCCCACCTGTGTCGCCGAGTGTGGAGGGACAGTGAGAGGAGAGGTGTCGGGGCAGGTGCT
GTCACCCGGGTATCCAGCTCCCTATGAACACAATCTCAACTGCATCTGGACCATCGAAGCAGAGGCCG
GCTGCACCATTGGGCTACACTTCCTGGTGTTTGACACAGAGGAGGTTCACGACGTGCTGCGCATCTGG
GATGGGCCTGTGGAGAGCGGGGTTCTGCTGAAGGAGCTGAGTGGCCCGGCCCTGCCCAAGGACCTGCA
TAGCACCTTCAACTCGGTCGTCCTGCAGTTCAGCACTGACTTCTTCACCAGCAAGCAGGGCTTTGCCA
TTCAATTTTCAGTGTCCACAGCAACGTCCTGCAATGACCCTGGGATCCCGCAGAATGGGAGTCGGAGT
GGTGACAGTTGGGAAGCCGGCGACTCCACAGTGTTCCAGTGTGACCCTGGCTACGCGCTGCAGGGAAG
TGCAGAGATCAGCTGTGTGAAGATCGAGAACAGGTTCTTCTGGCAGCCCAGCCCGCCAACATGCATCG
CTCCCTGCGGGGGAGACCTGACAGGACCATCTGGAGTCATCCTCTCACCAAATTACCCAGAACCCTAC
CCGCCAGGCAAGGAGTGTGACTGGAAAGTGACCGTCTCACCAGACTACGTCATCGCCCTGGTATTTAA
CATCTTTAACCTGGAGCCTGGCTATGACTTCCTCCATATCTACGACGGACGGGACTCTCTCAGCCCTC
TCATAGGAAGCTTCTATGGCTCCCAGCTCCCAGGCCGCATTGAAAGCAGCAGCAACAGCCTCTTCCTC
GCCTTCCGCAGCGATGCATCTGTGAGCAATGCTGGCTTCGTCATTGACTATACAGAAAACCCGCGGGA
GTCATGTTTTGATCCTGGTTCCATCAAGAACGGCACACGGGTGGGGTCCGACCTGAAGCTGGGCTCCT
CCGTCACCTACTACTGCCACGGGGGCTACGAAGTTGAGGGCACCTCGACCCTGAGCTGCATCCTGGGG
CCTGATGGGAAGCCCGTGTGGAACAATCCCCGGCCAGTCTGCACAGCCCCCTGTGGGGGACAGTATGT
GGGTTCGGACGGAGTGGTCTTGTCCCCCAACTACCCCCAGAACTACACCAGTGGACAGATCTGCTTGT
ATTTTGTTACTGTGCCCAAGGACTATGTGGTGTTTGGCCAGTTCGCCTTCTTTCACACGGCCCTCAAC
GACGTGGTGGAGGTTCACGACGGCCACAGCCAGCACTCGCGGCTCCTCAGCTCCCTCTCGGGCTCCCA
TACAGGAGAATCACTGCCCTTGGCCACCTCCAATCAAGTTCTCATTAAGTTCAGCGCCAAAGGCCTCG
CACCAGCCAGAGGCTTCCACTTTGTCTACCAAGCGGTTCCTCGAACCAGCGCCACGCAGTGCAGCTCT
GTGCCGGAACCCCGCTATGGCAAGAGGCTGGGCAGTGACTTCTCGGTGGGGGCCATCGTCCGCTTCGA
ATGCAACTCCGGCTATGCCCTGCAGGGGTCGCCAGAGATCGAGTGCCTCCCTGTGCCTGGGGCCTTGG
CCCAATGGAATGTCTCAGCGCCCACGTGTGTGGTGCCGTGTGGAGGCAACCTCACAGAGCGCAGGGGC
ACCATCCTGTCCCCTGGCTTCCCAGAGCCGTACCTCAACAGCCTCAACTGTGTGTGGAAGATCGTGGT
CCCCGAAGGCGCTGGCATCCAGATCCAAGTTGTCAGTTTTGTGACAGAGCAGAACTGGGACTCGCTGG
AAGTATTTGATGGTGCAGATAACACTGTAACCATGCTGGGGAGTTTCTCAGGAACAACCGTGCCTGCC
CTTCTGAACAGCACCTCCAACCAGCTCTACCTTCATTTCTACTCAGATATCAGCGTATCTGCAGCTGG
CTTCCACTTGGAGTACAAAACGGTGGGCCTGAGCAGTTGTCCGGAACCTGCTGTGCCCAGTAACGGGG
TGAAGACTGGCGAGCGCTACTTGGTGAATGATGTGGTGTCTTTCCAGTGTGAGCCGGGATATGCCCTC
CAGGGCCACGCCCACATCTCCTGCATGCCCGGAACAGTGCGGCGATGGAACTACCCTCCTCCACTCTG
TATTGCACAGTGTGGGGGAACAGTGGAGGAGATGGAGGGGGTGATCCTGAGCCCCGGCTTCCCAGGCA
ACTACCCCAGTAACATGGACTGCTCCTGGAAAATAGCACTGCCCGTGGGCTTTGGAGCTCACATCCAG
TTCCTGAACTTCTCCACCGAGCCCAACCACGACTACATAGAAATCCGGAATGGCCCCTATGAGACCAG
CCGCATGATGGGAAGATTCAGTGGAAGCGAGCTTCCAAGCTCCCTCCTCTCCACGTCCCACGAGACCA
CCGTGTATTTCCACAGCGACCACTCCCAGAATCGGCCAGGATTCAAGCTGGAGTATCAGGCCTATGAA
CTTCAAGAGTGCCCAGACCCAGAGCCCTTTGCCAATGGCATTGTGAGGGGAGCTGGCTACAACGTGGG
ACAATCAGTGACCTTCGAGTGCCTCCCGGGGTATCAATTGACTGGCCACCCTGTCCTCACGTGTCAAC
ATGGCACCAACCGGAACTGGGACCACCCCCTGCCCAAGTGTGAAGTCCCTTGTGGCGGGAACATCACT
TCTTCCAACGGCACTGTGTACTCCCCGGGGTTCCCTAGCCCGTACTCCAGCTCCCAGGACTGTGTCTG
GCTGATCACCGTGCCCATTGGCCATGGCGTCCGCCTCAACCTCAGCCTGCTGCAGACAGAGCCCTCTG
GAGATTTCATCACCATCTGGGATGGGCCACAGCAAACAGCACCACGGCTCGGCGTCTTCACCCGGAGC
ATGGCCAAGAAAACAGTGCAGAGTTCATCCAACCAGGTCCTGCTCAAGTTCCACCGTGATGCAGCCAC
AGGGGGGATCTTCGCCATAGCTTTCTCCGCTTATCCACTCACCAAATGCCCTCCTCCCACCATCCTCC
CCAACGCCGAAGTCGTCACAGAGAATGAAGAATTCAATATAGGTGACATCGTACGCTACAGATGCCTC
CCTGGCTTTACCTTAGTGGGGAATGAAATTCTGACCTGCAAACTTGGAACCTACCTGCAGTTTGAAGG
ACCACCCCCGATATGTGAAGTGCACTGTCCAACAAATGAGCTTCTGACAGACTCCACAGGCGTGATCC
TGAGCCAGAGCTACCCTGGAAGCTATCCCCAGTTCCAGACCTGCTCTTGGCTGGTGAGAGTGGAGCCC
GACTATAACATCTCCCTCACAGTGGAGTACTTCCTCAGCGAGAAGCAATATGATGAGTTTGAGATTTT
TGATGGTCCATCAGGACAGAGTCCTCTGCTGAAAGCCCTCAGTGGGAATTACTCAGCTCCCCTGATTG
TCACCAGCTCAAGCAACTCTGTGTACCTGCGTTGGTCATCTGATCACGCCTACAATCGGAAGGGCTTC
AAGATCCGCTATTCAGCCCCTTACTGCAGCCTGCCCAGGGCTCCACTCCATGGCTTCATCCTAGGCCA
GACCAGCACCCAGCCCGGGGGCTCCATCCACTTTGGCTGCAACGCCGGCTACCGCCTGGTGGGACACA
GCATGGCCATCTGTACCCGGCACCCCCAGGGCTACCACCTGTGGAGCGAAGCCATCCCTCTCTGTCAA
GCTCTTTCCTGTGGGCTTCCTGAGGCCCCCAAGAATGGAATGGTGTTTGGCAAGGAGTACACAGTGGG
AACCAAGGCCGTGTACAGCTGCAGTGAAGGCTACCACCTCCAGGCAGGCGCTGAGGCCACTGCAGAGT
GTCTGGACACAGGCCTATGGAGCAACCGCAATGTCCCACCACAGTGTGTCCCTGTGACTTGTCCTGAT
GTCAGTAGCATCAGCGTGGAGCATGGCCGATGGAGGCTTATCTTTGAGACACAGTATCAGTTCCAGGC
CCAGCTGATGCTCATCTGTGACCCTGGCTACTACTATACTGGCCAAAGGGTCATCCGCTGTCAGGCCA
ATGGCAAATGGAGCCTCGGGGACTCTACGCCCACCTGCCGAATCATCTCCTGTGGAGAGCTCCCGATT
CCCCCCAATGGCCACCGCATCGGAACACTGTCTGTCTACGGGGCAACAGCCATCTTCTCCTGCAATTC
CGGATACACACTGGTGGGCTCCAGGGTGCGTGAGTGCATGGCCAATGGGCTCTGGAGTGGCTCTGAAG
TCCGCTGCCTTGCTGGACACTGTGGGACTCCTGAGCCCATTGTCAACGGACACATCAATGGGGAGAAC
TACAGCTACCGGGGCAGTGTGGTGTACCAATGCAATGCTGGCTTCCGCCTGATCGGCATGTCTGTGCG
CATCTGCCAGCAGGATCATCACTGGTCGGGCAAGACCCCTTTCTGTGTGCCAATTACCTGTGGACACC
CAGGCAACCCTGTCAACGGCCTCACTCAGGGTAACCAGTTTAACCTCAACGATGTGGTCAAGTTTGTT
TGCAACCCTGGGTATATGGCTGAGGGGGCTGCTAGGTCCCAATGCCTGGCCAGCGGGCAATGGAGTGA
CATGCTGCCCACCTGCAGAATCATCAACTGTACAGATCCTGGACACCAAGAAAATAGTGTTCGTCAGG
TCCACGCCAGCGGCCCGCACAGGTTCAGCTTCGGCACCACTGTGTCTTACCGGTGCAACCACGGCTTC
TACCTCCTGGGCACCCCAGTGCTCAGCTGCCAGGGAGATGGCACATGGGACCGTCCCCGCCCCCAGTG
TCTCTTGGTGTCCTGTGGCCATCCGGGCTCCCCGCCTCACTCCCAGATGTCTGGAGACAGTTATACTG
TGGGAGCAGTGGTGCGGTACAGCTGCATCGGCAAGCGTACTCTGGTGGGAAACAGCACCCGCATGTGT
GGGCTGGATGGACACTGGACTGGCTCCCTCCCTCACTGCTCAGGAACCAGCGTGGGAGTTTGCGGTGA
CCCTGGGATCCCGGCTCATGGCATCCGTTTGGGGGACAGCTTTGATCCAGGCACTGTGATGCGCTTCA
GCTGTGAAGCTGGCCACGTGCTCCGGGGATCGTCAGAGCGCACCTGTCAAGCCAATGGCTCGTGGAGC
GGCTCGCAGCCTGAGTGTGGAGTGATCTCTTGTGGGAACCCTGGGACTCCAAGTAATGCCCGAGTTGT
GTTCAGTGATGGCCTGGTTTTCTCCAGCTCTATCGTCTATGAGTGCCGGGAAGGATACTACGCCACAG
GCCTGCTCAGCCGTCACTGCTCGGTCAATGGTACCTGGACAGGCAGTGACCCTGAGTGCCTCGTCATA
AACTGTGGTGACCCTGGGATTCCAGCCAATGGCCTTCGGCTGGGCAATGACTTCAGGTACAACAAAAC
TGTGACATATCAGTGTGTCCCTGGCTATATGATGGAGTCACATAGAGTATCTGTGCTGAGCTGCACCA
AGGACCGGACATGGAATGGAACCAAGCCCGTCTGCAAAGCTCTCATGTGCAAGCCACCTCCGCTCATC
CCCAATGGGAAGGTGGTGGGGTCTGACTTCATGTGGGGCTCAAGTGTGACTTATGCCTGCCTGGAGGG
GTACCAGCTCTCCCTGCCCGCGGTGTTCACCTGTGAGGGAAATGGGTCCTGGACCGGAGAGCTGCCTC
AGTGTTTCCCTGTGTTCTGCGGGGATCCTGGTGTCCCGTCCCGTGGGAGGAGAGAGGACCGAGGCTTC
TCCTACAGGTCATCTGTCTCCTTCTCCTGCCATCCCCCTCTGGTGCTGGTGGGCTCTCCACGCAGGTT
TTGCCAGTCAGATGGGACATGGAGTGGCACCCAGCCCAGCTGCATAGATCCGACCCTGACCACGTGTG
CGGACCCTGGTGTGCCACAGTTTGGGATACAGAACAATTCTCAGGGCTACCAGGTTGGAAGCACAGTC
CTCTTCCGTTGTCAAAAAGGCTACCTGCTTCAGGGCTCCACCACCAGGACCTGCCTCCCAAACCTGAC
CTGGAGTGGAACCCCACCTGACTGTGTCCCCCACCACTGCAGGCAGCCAGAGACGCCAACGCATGCCA
ACGTCGGGGCCCTGGATTTGCCCTCCATGGGCTACACGCTCATTACTCCTGCCAGGAGGGCTTCTCCC
TCAAGGGTGGCTCCGAGCACCGCACCTGCAAGGCGGATGGCAGCTGGACAGGCAAGCCGCCCATCTGC
CTGGAGGTCCGGCCCAGTGGGAGACCCATCAACACTGCCCGGGAGCCACCGCTCACCCAAGCCTTGAT
TCCTGGGGATGTTTTTGCCAAGAATTCCCTGTGGAAAGGGGCCTATGAATACCAGGGGAAGAAGCAGC
CAGCCATGCTCAGAGTGACTGGCTTCCAAGTTGCCAACAGCAAGGTCAATGCCACCATGATCGACCAC
AGTGGCGTGGAGCTGCACTTGGCTGGAACTTACAAGAAAGAAGATTTTCATCTCCTACTCCAGGTGTA
CCAGATTACAGGGCCTGTGGAGATCTTTATGAATAAGTTCAAAGATGATCACTGGGCTTTAGATGGCC
ATGTCTCGTCAGAGTCCTCCGGAGCCACCTTCATCTACCAAGGCTCTGTCAAGGGCCAAGGCTTTGGG
CAGTTCGGCTTTCAAAGACTGGACCTCAGGCTGCTGGAGTCAGACCCCGAGTCCATTGGCCGCCACTT
TGCTTCCAACAGCAGCTCAGTGGCAGCCGCGATCCTGGTGCCTTTCATCGCCCTCATTATTGCGGGCT
TCGTGCTCTATCTCTACAAGCACAGGAGAAGACCCAAAGTTCCTTTCAATGGCTATGCTGGCCACGAG
AACACCAATGTTCGGGCCACATTTGAGAACCCAATGTACGACCGCAACATCCAGCCCACAGACATCAT
GGCCAGCGAGGCGGAGTTCACAGTCAGCACAGTGTGCACAGCAGTATAGCCACCCGGCCTGGCCGCTT
TTTTTGCTAGGTTGAACTGGTACTCCAGCAGCCGCCGAAGCTGGACTGTACTGCTGCCATCTCAGCTC
ACTGCAACCTCCCTGCCTGATTCCCCTGCCTCAGCCTGCCGAGTGCCTGCGATTGCAGGCGCGCACCG
CCAC

| | | | |
|---|---|---|---|
| Nov4b, CG50377-01 | SEQ ID NO: 34 | 3104 aa | MW at 336746.3kD |
| Protein Sequence | | | |

MAGAPPPALLLPCSLISDCCASNQRHSVGVGPSELVKKQIELKSRGVKLMPSKDNSQKTSVLTQVGVS
QGHNMCPDPGIPERGKRLGSDFRLGSSVQFTCNEGYDLQGSKRITCMKVSDMFAAWSDHRPVCRARMC
DAHLRGPSGIITSPNFPIQYDNNAHCVWIITALNPSKVIKLAFEEFDLERGYDTLTVGDGGQDGDQKT
VLYMSQNACSDSPHTPGSRIPESMSGDIWRQKWTVLEICRDISSSDARSGSVRKSPKTSNAVELVAPG
TEIEQGSCGDPGIPAYGRREGSRFHHGDTLKFECQPAFELVGQKAITCQKNNQWSAKKPGCVFSCFFN
FTSPSGVVLSPNYPEDYGNHLHCVWLILARPESRIHLAFNDIDVEPQFDFLVIKDGATAEAPVLGTFS
GNQLPSSITSSGHVARLEFQTDHSTGKRGFNITFTTFRHNECPDPGVPVNGKRFGDSLQLGSSISFLC
DEGFLGTQGSETITCVLKEGSVVWNSAVLRCEAPCGGHLTSPSGTILSPGWPGFYKDALSCAWVIEAQ
PGYPIKITFDRFKTEVNYDTLEVRDGRTYSAPLIGVYHGTQVPQFLISTSNYLYLLFSTDKSHSDIGF
QLRYETITLQSDHCLDPGIPVNGQRHGNDFYVGALVTFSCDSGYTLSDGEPLECEPNFQWSRALPSCE
ALCGGFIQGSSGTILSPGFPDFYPNNLNCTWIIETSHGKGVFFTFHTFHLESGHDYLLITENGSFTQP
LRQLTGSRLPAPISAGLYGNFTAQVRFISDFSMSYEGFNITFSEYDLEPCEEPEVPAYSIRKGLQFGV
GDTLTFSCFPGYRLEGTARITCLGGRRRLWSSPLPRCVAECGNSVTGTQGTLLSPNFPVNYNNNHECI
YSIQTQPGKGIQLKAPAFELSEGDVLKVYDGNNNSARLLGVFSHSEMMGVTLNSTSSSLWLDFITDAE
NTSKGFELHFSSFELIKCEDPGTPKFGYKVHDEGHFAGSSVSFSCDPGYSLRGSEELLCLSGERRTWD
RPLPTCVAECGGTVRGEVSGQVLSPGYPAPYEHNLNCIWTIEAEAGCTIGLHFLVFDTEEVHDVLRIW
DGPVESGVLLKELSGPALPKDLHSTFNSVVLQFSTDFFTSKQGFAIQFSVSTATSCNDPGIPQNCSRS
GDSWEAGDSTVFQCDPGYALQGSAEISCVKIENRFFWQPSPPTCIAPCGGDLTGPSGVILSPNYPEPY
PPGKECDWKVTVSPDYVIALVFNIFNLEPGYDFLHIYDGRDSLSPLIGSFYGSQLPGRIESSSNSLFL
AFRSDASVSNAGFVIDYTENPRESCFDPGSIKNGTRVGSDLKLGSSVTYYCHGGYEVEGTSTLSCILG
PDGKPVWNNPRPVCTAPCGGQYVGSDGVVLSPNYPQNYTSGQICLYFVTVPKDYVVFGQFAFFHTALN
DVVEVHDGHSQHSRLLSSLSGSHTGESLPLATSNQVLIKFSAKGLAPARGFHFVYQAVPRTSATQCSS
VPEPRYGKRLGSDFSVGAIVRFECNSGYALQGSPEIECLPVPGALAQWNVSAPTCVVPCGGNLTERRG
TILSPGFPEPYLNSLNCVWKIVVPEGAGIQIQVVSFVTEQNWDSLEVFDGADNTVTMLGSFSGTTVPA
LLNSTSNQLYLHFYSDISVSAAGFHLEYKTVGLSSCPEPAVPSNGVKTGERYLVNDVVSFQCEPGYAL
QGHAHISCMPGTVRRWNYPPPLCIAQCGGTVEENEGVILSPGFPGNYPSNNDCSWKIALPVGFGAHIQ
FLNFSTEPNHDYIEIRNGPYETSRMMGRFSGSELPSSLLSTSHETTVYFHSDHSQNRPGFKLEYQAYE
LQECPDPEPFANGIVRGAGYNVGQSVTFECLPGYQLTGHPVLTCQHGTNRNWDHPLPKCEVPCGGNIT
SSNGTVYSPGFPSPYSSSQDCVWLITVPIGHGVRLNLSLLQTEPSGDFITIWDGPQQTAPRLGVFTRS
MAKKTVQSSSNQVLLKFHRDAATGGIFAIAFSAYPLTKCPPPTILPNAEVVTENEEFNIGDIVRYRCL
PGFTLVGNEILTCKLGTYLQFEGPPPICEVHCPTNELLTDSTGVILSQSYPGSYPQFQTCSWLVRVEP
DYNISLTVEYFLSEKQYDEFEIFDGPSGQSPLLKALSGNYSAPLIVTSSSNSVYLRWSSDHAYNRKGF
KIRYSAPYCSLPRAPLHGFILGQTSTQPGGSIHFGCNAGYRLVGHSMAICTRHPQGYHLWSEAIPLCQ
ALSCGLPEAPKNGMVFGKEYTVGTKAVYSCSEGYHLQAGAEATAECLDTGLWSNRNVPPQCVPVTCPD
VSSISVEHGRWPLIFETQYQFQAQLMLICDPGYYYTGQRVIRCQANGKWSLGDSTPTCRIISCGELPI
PPNGHRIGTLSVYGATAIFSCNSGYTLVGSRVRECMANGLWSGSEVRCLAGHCGTPEPIVNGHINGEN
YSYRGSVVYQCNAGFRLIGMSVRICQQDHHWSGKTPFCVPITCGHPGNPVNGLTQGNQFNLNDVVKFV
CNPGYMAEGAARSQCLASGQWSDMLPTCRIINCTDPGHQENSVRQVHASGPHRFSFGTTVSYRCNHGF
YLLGTPVLSCQGDGTWDRPRPQCLLVSCGHPGSPPHSQMSGDSYTVGAVVRYSCIGKRTLVGNSTRMC
GLDGHWTGSLPHCSGTSVGVCGDPGIPAHGIRLGDSFDPGTVMRFSCEAGBVLRGSSERTCQANGSWS
GSQPECGVISCGNPGTPSNARVVFSDGLVFSSSIVYECREGYYATGLLSRHCSVNGTWTGSDPECLVI
NCGDPGIPANGLRLGNDFRYNKTVTYQCVPGYMMESHRVSVLSCTKDRTWNGTKPVCKALMCKPPPLI
PNGKVVGSDFMWGSSVTYACLEGYQLSLPAVFTCEGNGSWTGELPQCFPVFCGDPGVPSRGRREDRGF
SYRSSVSFSCHPPLVLVGSPRRFCQSDGTWSGTQPSCIDPTLTTCADPGVPQFGIQNNSQGYQVGSTV
LFRCQKGYLLQGSTTRTCLPNLTWSGTPPDCVPHHCRQPETPTHANVGALDLPSMGYTLITPARRASP
SRVAPSTAPARRMAAGQASRPSAWRSGPVGDPSTLPGSHRSPKP

| | | |
|---|---|---|
| NOV4c, CG50377-02 | SEQ ID NO: 35 | 8010 bp |
| DNA Sequence | ORF Start: ATG at 1 | ORF Stop: TAG at 8008 |

ATGGCGGGCGCCCCTCCCCCCGCCTTGCTGCTGCCTTGCAGTTTGATCTCAGACTGCTGTGCTAGCAA
TCAGCGACACTCCGTGGGCGTAGGACCCTCCGAGCTAGTCAAGAAGCAAATTGAGTTGAAGTCTCGAG
GTGTGAAGCTGATGCCCAGCAAAGACAACAGCCAGAAGACGTCTGTGTTAACTCAGGTTGGTGTGTCC
CAAGGACATAATATGTGTCCAGACCCTGGCATACCCGAAAGGGGCAAAAGACTAGGCTCGGATTTCAG
GTTAGGATCCAGCGTCCAGTTCACCTGCAACGAGGGCTATGACCTGCAAGGGTCCAAGCGGATCACCT
GTATGAAAGTGAGCGACATGTTTGCGGCCTGGAGCGACCACAGGCCAGTCTGCCGAGCCCGCATGTGT
GATGCCCACCTTCGAGGCCCCTCGGGCATCATCACCTCCCCCAATTTCCCCATTCAGTATGACAACAA
TGCACACTGTGTGTGGATCATCACAGCACTCAACCCCTCCAAGGTGATCAAGCTCGCCTTTGAGGAGT
TTGATTTGGAGAGGGGCTATGACACCCTGACGGTCGGTGATGGTGGTCAGGATGGGGACCAGAAGACA
GTTCTCTACATGTCTCAAAATGCCTGCAGTGACAGCCCTCACACCCCAGGCTCTCGCATCCCAGAGAG
CATGTCTGGGGACATCTGGAGGCAGAAATGGACTGTACTTGAGATCTGTCGTGACATTAGCAGTTCAG
ATGCAAGGTCAGGTTCAGTGAGGAAGTCTCCAAAGACTTCTAATGCTGTGGAACTTGTTGCTCCTGGG
ACAGAGATCGAGCAGGGCAGTTGCGGTGACCCTGGCATACCTGCATATGGCCGGAGGGAAGGCTCCCG
GTTTCACCACGGTGACACACTCAAGTTTGAGTGCCAGCCCGCCTTTGAGCTGGTGGGACAGAAGGCAA
TCACATGCCAAAAGAATAACCAATGGTCGGCTAAGAAGCCAGGCTGCGTGTTCTCCTGCTTCTTCAAC
TTCACCAGCCCGTCTGGGGTTGTCCTGTCTCCCAACTACCCAGAGGACTATGGCAACCACCTCCACTG
TGTCTGGCTCATCCTGGCCAGGCCTGAGAGCCGCATCCACCTGGCCTTCAACGACATTGACGTGGAGC
CTCAGTTTGATTTCCTGGTCATCAAGGATGGGGCCACCGCCGAGGCGCCCGTCCTGGGCACCTTCTCA
GGAAACCAGCTTCCCTCCTCCATCACAAGCAGTGGCCACGTGGCCCGTCTCGAGTTCCAGACTGACCA
CTCCACAGGGAAGAGGGGCTTCAACATCACTTTTACCACCTTCCGACACAACGAGTGCCCGGATCCTG
GCGTTCCAGTAAATGGCAAACGGTTTGGGGACAGCCTCCAGCTGGGCAGCTCCATCTCCTTCCTCTGT
GATGAAGGCTTCCTTGGGACTCAGGGCTCAGAGACCATCACCTGCGTCCTGAAGGAGGGCAGCGTGGT
CTGGAACAGCGCTGTGCTGCGGTGTGAAGCTCCCTGTGGTGGTCACCTGACTTCGCCCAGCGGCACCA
TCCTCTCTCCGGGCTGGCCTGGCTTCTACAAGGATGCCTTGAGCTGTGCCTGGGTGATTGAGGCCCAG
CCAGGCTACCCCATCAAAATCACCTTCGACAGATTCAAAACCGAGGTCAACTATGACACCCTGGAAGT
ACGCGATGGGCGGACTTACTCAGCGCCCTTGATCGGGGTTTACCACGGGACCCAGGTTCCCCAGTTCC
TCATCAGCACCAGCAACTACCTCTACCTCCTCTTCTCTACCGACAAGAGTCACTCGGACATCGGCTTC
CAGCTCCGCTATGAGACTATAACACTGCAGTCAGACCACTGTCTGGATCCAGGAATCCCAGTAAATGG
ACAGCGTCATGGGAATGACTTCTACGTGGGCGCGCTGGTGACCTTCAGCTGTGACTCGGGCTACACAT
TAAGTGACGGGGAGCCTCTGGAGTGTGAGCCCAACTTCCAGTGGAGCCGGGCCCTGCCCAGTTGTGAA
GCTCTCTGTGGTGGCTTCATTCAAGGCTCCAGTGGGACCATCTTGTCGCCAGGGTTCCCTGACTTCTA
CCCCAACAACTTGAACTGCACCTGGATTATCGAAACATCTCATGGCAAGGGTGTGTTCTTCACTTTCC
ACACCTTCCACCTGGAAAGTGGCCATGACTACCTCCTCATCACTGAGAACGGCAGCTTCACCCAGCCC
CTGAGGCAGCTAACTGGATCTCGGCTGCCAGCTCCCATCAGCGCTGGGCTCTATGGCAACTTCACTGC
CCAGGTCCGCTTCATCTCTGATTTCTCCATGTCATATGAAGGATTCAACATCACCTTCTCAGAGTACG
ACTTGGAGCCCTGTGAGGAGCCCGAGGTCCCAGCCTACAGCATCCGGAAGGGCTTGCAGTTTGGCGTG
GGCGACACCTTGACCTTCTCCTGCTTCCCCGGGTACCGTCTGGAGGGCACCGCCCGCATCACGTGCCT
GGGGGGCAGACGGCGCCTGTGGAGCTCGCCTCTGCCAAGGTGTGTTGCTGAGTGTGGGAATTCAGTCA
CAGGCACTCAGGGTACTTTGCTGTCCCCCAACTTTCCTGTGAACTACAATAACAATCATGAATGCATC
TACTCCATCCAGACCCAGCCAGGGAAGGGAATTCAGCTGAAAGCCAGGGCATTCGAACTCTCCGAAGG
AGATGTCCTCAAGGTTTATGATGGCAACAACAACTCCGCCCGTTTGCTGGGAGTTTTTAGCCATTCTG
AGATGATGGGGGTGACTTTGAACAGCACATCCAGCAGTCTGTGGCTTGATTTCATCACTGATGCTGAA
AACACCAGCAAGGGCTTTGAACTGCACTTTTCCAGCTTTGAACTCATCAAATGTGAGGACCCAGGAAC
CCCCAAGTTTGGCTACAAGGTTCATGATGAAGGTCATTTTGCAGGGAGCTCCGTGTCCTTCAGCTGTG
ACCCTGGATACAGCCTGCGGGGTAGTGAGGAGCTGCTGTGTCTGAGTGGAGAGCGCCGGACCTGGGAC
CGGCCTCTGCCCACCTGTGTCGCCGAGTGTGGAGGGACAGTGAGAGGAGAGGTGTCGGGGCAGGTGCT
GTCACCCGGGTATCCAGCTCCCTATGAACACAATCTCAACTGCATCTGGACCATCGAAGCAGAGGCCG
GCTGCACCATTGGGCTACACTTCCTGGTGTTTGACACAGAGGAGGTTCACGACGTGCTGCGCATCTGG
GATGGGCCTGTGGAGAGCGGGGTTCTGCTGAAGGAGCTGAGTGGCCCGGCCCTGCCCAAGGACCTGCA
TAGCACCTTCAACTCGGTCGTCCTGCAGTTCAGCACTGACTTCTTCACCAGCAAGCAGGGCTTTGCCA
TTCAATTTTCAGTGTCCACAGCAACGTCCTGCAATGACCCTGGGATCCCGCAGAATGGGAGTCGGAGT
GGTGACAGTTGGGAAGCCGGCGACTCCACAGTGTTCCAGTGTGACCCTGGCTACGCGCTGCAGGGAAG
TGCAGAGATCAGCTGTGTGAAGATCGAGAACAGGTTCTTCTGGCAGCCCAGCCCGCCAACATGCATCG
CTCCCTGCGGGGGAGACCTGACAGGACCATCTGGAGTCATCCTCTCACCAAATTACCCAGAACCCTAC
CCGCCAGGCAAGGAGTGTGACTGGAAAGTGACCGTCTCACCAGACTACGTCATCGCCCTGGTATTTAA
CATCTTTAACCTGGAGCCTGGCTATGACTTCCTCCATATCTACGACGGACGGGACTCTCTCAGCCCTC
TCATAGGAAGCTTCTATGGCTCCCAGCTCCCAGGCCGCATTGAAAGCAGCAGCAACAGCCTCTTCCTC
GCCTTCCGCAGCGATGCATCTGTGAGCAATGCTGGCTTCGTCATTGACTATACAGAAAACCCGCGGGA
GTCATGTTTTGATCCTGGTTCCATCAAGAACGGCACACGGGTGGGGTCCGACCTGAAGCTGGGCTCCT
CCGTCACCTACTACTGCCACGGGGGCTACGAAGTTGAGGGCACCTCGACCCTGAGCTGCATCCTGGGG
CCTGATGGGAAGCCCGTGTGGAACAATCCCCGGCCAGTCTGCACAGCCCCCTGTGGGGGACAGTATGT
GGGTTCGGACGGAGTGGTCTTGTCCCCCAACTACCCCCAGAACTACACCAGTGGACAGATCTGCTTGT
ATTTTGTTACTGTGCCCAAGGACTATGTGGTGTTTGGCCAGTTCGCCTTCTTTCACACGGCCCTCAAC
GACGTGGTGGAGGTTCACGACGGCCACAGCCAGCACTCGCGGCTCCTCAGCTCCCTCTCGGGCTCCCA
TACAGGAGAATCACTGCCCTTGGCCACCTCCAATCAAGTTCTCATTAAGTTCAGCGCCAAAGGCCTCG
CACCAGCCAGAGGCTTCCACTTTGTCTACCAAGCGGTTCCTCGAACCAGCGCCACGCAGTGCAGCTCT
GTGCCGGAACCCCGCTATGGCAAGAGGCTGGGCAGTGACTTCTCGGTGGGGGCCATCGTCCGCTTCGA
ATGCAACTCCGGCTATGCCCTGCAGGGGTCGCCAGAGATCGAGTGCCTCCCTGTGCCTGGGGCCTTGG
CCCAATGGAATGTCTCAGCGCCCACGTGTGTGGTGCCGTGTGGAGGCAACCTCACAGAGCGCAGGGGC
ACCATCCTGTCCCCTGGCTTCCCAGAGCCGTACCTCAACAGCCTCAACTGTGTGTGGAAGATCGTGGT
CCCCGAAGGCGCTGGCATCCAGATCCAAGTTGTCAGTTTTGTGACAGAGCAGAACTGGGACTCGCTGG
AAGTATTTGATGGTGCAGATAACACTGTAACCATGCTGGGGAGTTTCTCAGGAACAACCGTGCCTGCC
CTTCTGAACAGCACCTCCAACCAGCTCTACCTTCATTTCTACTCAGATATCAGCGTATCTGCAGCTGG
CTTCCACTTGGAGTACAAAACGGTGGGCCTGAGCAGTTGTCCGGAACCTGCTGTGCCCAGTAACGGGG
TGAAGACTGGCGAGCGCTACTTGGTGAATGATGTGGTGTCTTTCCAGTGTGAGCCGGGATATGCCCTC
CAGGGCCACGCCCACATCTCCTGCATGCCCGGAACAGTGCGGCGATGGAACTACCCTCCTCCACTCTG
TATTGCACAGTGTGGGGGAACAGTGGAGGAGATGGAGGGGGTGATCCTGAGCCCCGGCTTCCCAGGCA
ACTACCCCAGTAACATGGACTGCTCCTGGAAAATAGCACTGCCCGTGGGCTTTGGAGCTCACATCCAG
TTCCTGAACTTCTCCACCGAGCCCAACCACGACTACATAGAAATCCGGAATGGCCCCTATGAGACCAG
CCGCATGATGGGAAGATTCAGTGGAAGCGAGCTTCCAAGCTCCCTCCTCTCCACGTCCCACGAGACCA
CCGTGTATTTCCACAGCGACCACTCCCAGAATCGGCCAGGATTCAAGCTGGAGTATCAGGCCTATGAA
CTTCAAGAGTGCCCAGACCCAGAGCCCTTTGCCAATGGCATTGTGAGGGGAGCTGGCTACAACGTGGG
ACAATCAGTGACCTTCGAGTGCCTCCCGGGGTATCAATTGACTGGCCACCCTGTCCTCACGTGTCAAC
ATGGCACCAACCGGAACTGGGACCACCCCCTGCCCAAGTGTGAAGTCCCTTGTGGCGGGAACATCACT
TCTTCCAACGGCACTGTGTACTCCCCGGGGTTCCCTAGCCCGTACTCCAGCTCCCAGGACTGTGTCTG
GCTGATCACCGTGCCCATTGGCCATGGCGTCCGCCTCAACCTCAGCCTGCTGCAGACAGAGCCCTCTG
GAGATTTCATCACCATCTGGGATGGGCCACAGCAAACAGCACCACGGCTCGGCGTCTTCACCCGGAGC
ATGGCCAAGAAAACAGTGCAGAGTTCATCCAACCAGGTCCTGCTCAAGTTCCACCGTGATGCAGCCAC
AGGGGGGATCTTCGCCATAGCTTTCTCCGCTTATCCACTCACCAAATGCCCTCCTCCCACCATCCTCC
CCAACGCCGAAGTCGTCACAGAGAATGAAGAATTCAATATAGGTGACATCGTACGCTACAGATGCCTC
CCTGGCTTTACCTTAGTGGGGAATGAAATTCTGACCTGCAAACTTGGAACCTACCTGCAGTTTGAAGG
ACCACCCCCGATATGTGAAGTGCACTGTCCAACAAATGAGCTTCTGACAGACTCCACAGGCGTGATCC
TGAGCCAGAGCTACCCTGGAAGCTATCCCCAGTTCCAGACCTGCTCTTGGCTGGTGAGAGTGGAGCCC
GACTATAACATCTCCCTCACAGTGGAGTACTTCCTCAGCGAGAAGCAATATGATGAGTTTGAGATTTT
TGATGGTCCATCAGGACAGAGTCCTCTGCTGAAAGCCCTCAGTGGGAATTACTCAGCTCCCCTGATTG
TCACCAGCTCAAGCAACTCTGTGTACCTGCGTTGGTCATCTGATCACGCCTACAATCGGAAGGGCTTC
AAGATCCGCTATTCAGCCCCTTACTGCAGCCTGCCCAGGGCTCCACTCCATGGCTTCATCCTAGGCCA
GACCAGCACCCAGCCCGGGGGCTCCATCCACTTTGGCTGCAACGCCGGCTACCGCCTGGTGGGACACA
GCATGGCCATCTGTACCCGGCACCCCCAGGGCTACCACCTGTGGAGCGAAGCCATCCCTCTCTGTCAA
GCTCTTTCCTGTGGGCTTCCTGAGGCCCCCAAGAATGGAATGGTGTTTGGCAAGGAGTACACAGTGGG
AACCAAGGCCATGTACAGCTGCAGTGAAGGCTACCACCTCCAGGCAGGCGCTGAGGCCACTGCAGAGT
GTCTGGACACAGGCCTATGGAGCAACCGCAATGTCCCACCACAGTGTGTCCGTGAGTCCTCGGGCAAT
GGAGGCGGGTCTGTGACTTGTCCTGATGTCAGTAGCATCAGCGTGGAGCATGGCCGATGGAGGCTTAT
CTTTGAGACACAGTATCAGTTCCAGGCCCAGCTGATGCTCATCTGTGACCCTGGCTACTACTATACTG
GCCAAAGGGTCATCCGCTGTCAGGCCAATGGCAAATGGAGCCTCGGGGACTCTACGCCCACCTGCCGA
ATCATCTCCTGTGGAGAGCTCCCGATTCCCCCCAATGGCCACCGCATCGGAACACTGTCTGTCTACGG
GGCAACAGCCATCTTCTCCTGCAATTCCGGATACACACTGGTGGGCTCCAGGGTGCGTGAGTGCATGG
CCAATGGGCTCTGGAGTGGCTCTGAAGTCCGCTGCCTTGCCACTCAGACCAAGCTCCACTCCATTTTC
TATAAGCTCCTCTTCGATGTACTCTCTTCCCCATCCCTCACCAAAGCTGGACACTGTGGGACTCCTGA
GCCCATTGTCAACGGACACATCAATGGGGAGAACTACAGCTACCGGGGCAGTGTGGTGTACCAATGCA
ATGCTGGCTTCCGCCTGATCGGCATGTCTGTGCGCATCTGCCAGCAGGATCATCACTGGTCGGGCAAG
ACCCCTTTCTGTGTGCATGTTAAGCAGCAGTTGCTGCTGCTGCTGCTGCTGTTGTGTGATGATGATGA
TGATGAAGATGATGGTAGTGGTGCAATTACCTGTGGACACCCAGGCAACCCTGTCAACGGCCTCACTC
AGGGTAACCAGTTTAACCTCAACGATGTGGTCAAGTTTGTTTGCAACCCTGGGTATATGGCTGAGGGG
GCTGCTAGGTCCCAATGCCTGGCCAGCGGGCAATGGAGTGACATGCTGCCCACCTGCAGAATCATCAA
CTGTACAGATCCTGGACACCAAGAAAATAGTGTTCGTCAGGTCCACGCCAGCGGCCCGCACAGGTTCA
GCTTCGGCACCACTGTGTCTTACCGGTGCAACCACGGCTTCTACCTCCTGGGCACCCCAGTGCTCAGC
TGCCAGGGAGATGGCACATGGGACCGTCCCCGCCCCCAGTGTCTCTGTAAGTAG

| | | | |
|---|---|---|---|
| NOV4c, CG50377-02 | SEQ ID NO: 36 | 2669 aa | MW at 290805.9kD |
| Protein Sequence | | | |

MAGAPPPALLLPCSLISDCCASNQRHSVGVGPSELVKKQIELKSRGVKLMPSKDNSQKTSVLTQVGVS
QGHNMCPDPGIPERGKRLGSDFRLGSSVQFTCNEGYDLQGSKRITCMKVSDMFAAWSDHRPVCPARMC
DAHLRGPSGIITSPNFPIQYDNNAHCVWIITALNPSKVIKLAFEEFDLERGYDTLTVGDGGQDGDQKT
VLYMSQNACSDSPHTPGSRIPESMSGDIWRQKWTVLEICRDISSSDARSGSVRKSPKTSNAVELVAPG
TEIEQGSCGDPGIPAYGRREGSRFHHGDTLKFECQPAFELVGQKAITCQKNNQWSAKKPGCVFSCFFN
FTSPSGVVLSPNYPEDYGNHLHCVWLILARPESRIHLAFNDIDVEPQFDFLVIKDGATAEAPVLGTFS
GNQLPSSITSSGHVARLEFQTDHSTGKRGFNITFTTFRHNECPDPGVPVNGKRFGDSLQLGSSISFLC
DEGFLGTQGSETITCVLKEGSVVWNSAVLRCEAPCGGHLTSPSGTILSPGWPGFYKDALSCAWVIEAQ
PGYPIKITFDRFKTEVNYDTLEVRDGRTYSAPLIGVYHGTQVPQFLISTSNYLYLLFSTDKSHSDIGF
QLRYETITLQSDHCLDPGIPVNGQRHGNDFYVGALVTFSCDSGYTLSDGEPLECEPNFQWSPALPSCE
ALCGGFIQGSSGTILSPGFPDFYPNNLNCTWIIETSHGKGVFFTFHTFHLESGHDYLLITENGSFTQP
LRQLTGSRLPAPISAGLYGNFTAQVRFISDFSMSYEGFNITFSEYDLEPCEEPEVPAYSIRKGLQFGV
GDTLTFSCFPGYRLEGTARITCLGGRRRLWSSPLPRCVAECGNSVTGTQGTLLSPNFPVNYNNNHECI
YSIQTQPGKGIQLKAPAFELSEGDVLKVYDGNNNSARLLGVFSHSENMGVTLNSTSSSLWLDFITDAE
NTSKGFELHFSSFELIKCEDPGTPKFGYKVHDEGHFAGSSVSFSCDPGYSLRGSEELLCLSGERRTWD
RPLPTCVAECGGTVRGEVSGQVLSPGYPAPYEHNLNCIWTIEAEAGCTIGLHFLVFDTEEVHDVLRIW
DGPVESGVLLKELSGPALPKDLHSTFNSVVLQFSTDFFTSKQGFAIQFSVSTATSCNDPGIPQNGSRS
GDSWEAGDSTVFQCDPGYALQGSAEISCVKIENRFFWQPSPPTCIAPCGGDLTGPSGVILSPNYPEPY
PPGKECDWKVTVSPDYVIALVFNIFNLEPGYDFLHIYDGRDSLSPLIGSFYGSQLPGRIESSSNSLFL
AFRSDASVSNAGFVIDYTENPRESCFDPGSIKNGTRVGSDLKLGSSVTYYCHGGYEVEGTSTLSCILG
PDGKPVWNNPRPVCTAPCGGQYVGSDGVVLSPNYPQNYTSGQICLYFVTVPKDYVVFGQFAFFHTALN
DVVEVHDGHSQHSRLLSSLSGSHTGESLPLATSNQVLIKFSAKGLAPARGFHFVYQAVPRTSATQCSS
VPEPRYGKRLGSDFSVGAIVRFECNSGYALQGSPEIECLPVPGAIAQWNVSAPTCVVPCGGNLTERRG
TILSPGFPEPYLNSLNCVWKIVVPEGAGIQIQVVSFVTEQNWDSLEVFDGADNTVTMLGSFSGTTVPA
LLNSTSNQLYLHFYSDISVSAAGFHLEYKTVGLSSCPEPAVPSNGVKTGERYLVNDVVSFQCEPGYAL
QGHAHISCMPGTVRRWNYPPPLCIAQCGGTVEEMEGVILSPGFPGNYPSNMDCSWKIALPVGFGAHIQ
FLNFSTEPNHDYIEIRNGPYETSRMMGRFSGSELPSSLLSTSHETTVYFHSDHSQNRPGFKLEYQAYE
LQECPDPEPFANGIVRGAGYNVGQSVTFECLPGYQLTGHPVLTCQHGTNRNWDHPLPKCEVPCGGNIT
SSNGTVYSPGFPSPYSSSQDCVWLITVPIGHGVRLNLSLLQTEPSGDFITIWDGPQQTAPRLGVFTRS
MAKKTVQSSSNQVLLKFHRDAATGGIFAIAFSAYPLTKCPPPTILPNAEVVTENEEFNIGDIVRYRCL
PGFTLVGNEILTCKLGTYLQFEGPPPICEVHCPTNELLTDSTGVILSQSYPGSYPQFQTCSWLVRVEP
DYNISLTVEYFLSEKQYDEFEIFDGPSGQSPLLKALSGNYSAPLIVTSSSNSVYLRWSSDHAYNRKGF
KIRYSAPYCSLPRAPLHGFILGQTSTQPGGSIHFGCNAGYRLVGHSMAICTRHPQGYHLWSEAIPLCQ
ALSCGLPEAPKNGMVFGKEYTVGTKAMYSCSEGYHLQAGAEATAECLDTGLWSNRNVPPQCVRESSGN
GGGSVTCPDVSSISVEHGRWRLIFETQYQFQAQLMLICDPGYYYTGQRVIRCQANGKWSLGDSTPTCR
IISCGELPIPPNGHRIGTLSVYGATAIFSCNSGYTLVGSRVRECMANGLWSGSEVRCLATQTKLHSIF
YKLLFDVLSSPSLTKAGHCGTPEPIVNGHINGENYSYRGSVVYQCNAGFRLIGMSVRICQQDHHWSGK
TPFCVHVKQQLLLLLLLLCDDDDDEDDGSGAITCGHPGNPVNGLTQGNQFNLNDVVKFVCNPGYMAEG
AARSQCLASGQWSDMLPTCRIINCTDPGHQENSVRQVHASGPHRFSFGTTVSYRCNHGFYLLGTPVLS
CQGDGTWDRPRPQCLCK

| | | |
|---|---|---|
| NOV4d, CG50377-03 | SEQ ID NO: 37 | 9951 bp |
| DNA Sequence | ORF Start: ATG at 1 | ORf Stop: TGA at 7837 |

ATGGCGGGCGCCCCTCCCCCCGCCTTGCTGCTGCCTTGCAGTTTGATCTCAGACTGCTGTGCTAGCAA
TCAGCGACACTCCGTGGGCGTAGGACCCTCCGAGCTAGTCAAGAAGCAAATTGAGTTGAAGTCTCGAG
GTGTGAAGCTGATGCCCAGCAAAGACAACAGCCAGAAGACGTCTGTGTTAACTCAGGTTGGTGTGTCC
CAAGGACATAATATGTGTCCAGACCCTGGCATACCCGAAAGGGGCAAAAGACTAGGCTCGGATTTCAG
GTTAGGATCCAGCGTCCAGTTCACCTGCAACGAGGGCTATGACCTGCAAGGGTCCAAGCGGATCACCT
GTATGAAAGTGAGCGACATGTTTGCGGCCTGGAGCGACCACAGGCCAGTCTGCCGAGCCCGCATGTGT
GATGCCCACCTTCGAGGCCCCTCGGGCATCATCACCTCCCCCAATTTCCCCATTCAGTATGACAACAA
TGCACACTGTGTGTGGATCATCACAGCACTCAACCCCTCCAAGGTGATCAAGCTCGCCTTTGAGGAGT
TTGATTTGGAGAGGGGCTATGACACCCTGACGGTCGGTGATGGTGGTCAGGATGGGGACCAGAAGACA
GTTCTCTACATGTCTCAAAATGCCTGCAGTGACAGCCCTCACACCCCAGGCTCTCGCATCCCAGAGAG
CATGTCTGGGGACATCTGGAGGCAGAAATGGACTGTACTTGAGATCTGTCGTGACATTAGCAGTTCAG
ATGCAAGGTCAGGTTCAGTGAGGAAGTCTCCAAAGACTTCTAATGCTGTGGAACTTGTTGCTCCTGGG
ACAGAGATCGAGCAGGGCAGTTGCGGTGACCCTGGCATACCTGCATATGGCCGGAGGGAAGGCTCCCG
GTTTCACCACGGTGACACACTCAAGTTTGAGTGCCAGCCCGCCTTTGAGCTGGTGGGACAGAAGGCAA
TCACATGCCAAAAGAATAACCAATGGTCGGCTAAGAAGCCAGGCTGCGTGTTCTCCTGCTTCTTCAAC
TTCACCAGCCCGTCTGGGGTTGTCCTGTCTCCCAACTACCCAGAGGACTATGGCAACCACCTCCACTG
TGTCTGGCTCATCCTGGCCAGGCCTGAGAGCCGCATCCACCTGGCCTTCAACGACATTGACGTGGAGC
CTCAGTTTGATTTCCTGGTCATCAAGGATGGGGCCACCGCCGAGGCGCCCGTCCTGGGCACCTTCTCA
GGAAACCAGCTTCCCTCCTCCATCACAAGCAGTGGCCACGTGGCCCGTCTCGAGTTCCAGACTGACCA
CTCCACAGGGAAGAGGGGCTTCAACATCACTTTTACCACCTTCCGACACAACGAGTGCCCGGATCCTG
GCGTTCCAGTAAATGGCAAACGGTTTGGGGACAGCCTCCAGCTGGGCAGCTCCATCTCCTTCCTCTGT
GATGAAGGCTTCCTTGGGACTCAGGGCTCAGAGACCATCACCTGCGTCCTGAAGGAGGGCAGCGTGGT
CTGGAACAGCGCTGTGCTGCGGTGTGAAGCTCCCTGTGGTGGTCACCTGACTTCGCCCAGCGGCACCA
TCCTCTCTCCGGGCTGGCCTGGCTTCTACAAGGATGCCTTGAGCTGTGCCTGGGTGATTGAGGCCCAG
CCAGGCTACCCCATCAAAATCACCTTCGACAGATTCAAAACCGAGGTCAACTATGACACCCTGGAAGT
ACGCGATGGGCGGACTTACTCAGCGCCCTTGATCGGGGTTTACCACGGGACCCAGGTTCCCCAGTTCC
TCATCAGCACCAGCAACTACCTCTACCTCCTCTTCTCTACCGACAAGAGTCACTCGGACATCGGCTTC
CAGCTCCGCTATGAGACTATAACACTGCAGTCAGACCACTGTCTGGATCCAGGAATCCCAGTAAATGG
ACAGCGTCATGGGAATGACTTCTACGTGGGCGCGCTGGTGACCTTCAGCTGTGACTCGGGCTACACAT
TAAGTGACGGGGAGCCTCTGGAGTGTGAGCCCAACTTCCAGTGGAGCCGGGCCCTGCCCAGTTGTGAA
GCTCTCTGTGGTGGCTTCATTCAAGGCTCCAGTGGGACCATCTTGTCGCCAGGGTTCCCTGACTTCTA
CCCCAACAACTTGAACTGCACCTGGATTATCGAAACATCTCATGGCAAGGGTGTGTTCTTCACTTTCC
ACACCTTCCACCTGGAAAGTGGCCATGACTACCTCCTCATCACTGAGAACGGCAGCTTCACCCAGCCC
CTGAGGCAGCTAACTGGATCTCGGCTGCCAGCTCCCATCAGCGCTGGGCTCTATGGCAACTTCACTGC
CCAGGTCCGCTTCATCTCTGATTTCTCCATGTCATATGAAGGATTCAACATCACCTTCTCAGAGTACG
ACTTGGAGCCCTGTGAGGAGCCCGAGGTCCCAGCCTACAGCATCCGGAAGGGCTTGCAGTTTGGCGTG
GGCGACACCTTGACCTTCTCCTGCTTCCCCGGGTACCGTCTGGAGGGCACCGCCCGCATCACGTGCCT
GGGGGGCAGACGGCGCCTGTGGAGCTCGCCTCTGCCAAGGTGTGTTGCTGAGTGTGGGAATTCAGTCA
CAGGCACTCAGGGTACTTTGCTGTCCCCCAACTTTCCTGTGAACTACAATAACAATCATGAATGCATC
TACTCCATCCAGACCCAGCCAGGGAAGGGAATTCAGCTGAAAGCCAGGGCATTCGAACTCTCCGAAGG
AGATGTCCTCAAGGTTTATGATGGCAACAACAACTCCGCCCGTTTGCTGGGAGTTTTTAGCCATTCTG
AGATGATGGGGGTGACTTTGAACAGCACATCCAGCAGTCTGTGGCTTGATTTCATCACTGATGCTGAA
AACACCAGCAAGGGCTTTGAACTGCACTTTTCCAGCTTTGAACTCATCAAATGTGAGGACCCAGGAAC
CCCCAAGTTTGGCTACAAGGTTCATGATGAAGGTCATTTTGCAGGGAGCTCCGTGTCCTTCAGCTGTG
ACCCTGGATACAGCCTGCGGGGTAGTGAGGAGCTGCTGTGTCTGAGTGGAGAGCGCCGGACCTGGGAC
CGGCCTCTGCCCACCTGTGTCGCCGAGTGTGGAGGGACAGTGAGAGGAGAGGTGTCGGGGCAGGTGCT
GTCACCCGGGTATCCAGCTCCCTATGAACACAATCTCAACTGCATCTGGACCATCGAAGCAGAGGCCG
GCTGCACCATTGGGCTACACTTCCTGGTGTTTGACACAGAGGAGGTTCACGACGTGCTGCGCATCTGG
GATGGGCCTGTGGAGAGCGGGGTTCTGCTGAAGGAGCTGAGTGGCCCGGCCCTGCCCAAGGACCTGCA
TAGCACCTTCAACTCGGTCGTCCTGCAGTTCAGCACTGACTTCTTCACCAGCAAGCAGGGCTTTGCCA
TTCAATTTTCAGTGTCCACAGCAACGTCCTGCAATGACCCTGGGATCCCGCAGAATGGGAGTCGGAGT
GGTGACAGTTGGGAAGCCGGCGACTCCACAGTGTTCCAGTGTGACCCTGGCTACGCGCTGCAGGGAAG
TGCAGAGATCAGCTGTGTGAAGATCGAGAACAGGTTCTTCTGGCAGCCCAGCCCGCCAACATGCATCG
CTCCCTGCGGGGGAGACCTGACAGGACCATCTGGAGTCATCCTCTCACCAAATTACCCAGAACCCTAC
CCGCCAGGCAAGGAGTGTGACTGGAAAGTGACCGTCTCACCAGACTACGTCATCGCCCTGGTATTTAA
CATCTTTAACCTGGAGCCTGGCTATGACTTCCTCCATATCTACGACGGACGGGACTCTCTCAGCCCTC
TCATAGGAAGCTTCTATGGCTCCCAGCTCCCAGGCCGCATTGAAAGCAGCAGCAACAGCCTCTTCCTC
GCCTTCCGCAGCGATGCATCTGTGAGCAATGCTGGCTTCGTCATTGACTATACAGAAAACCCGCGGGA
GTCATGTTTTGATCCTGGTTCCATCAAGAACGGCACACGGGTGGGGTCCGACCTGAAGCTGGGCTCCT
CCGTCACCTACTACTGCCACGGGGGCTACGAAGTTGAGGGCACCTCGACCCTGAGCTGCATCCTGGGG
CCTGATGGGAAGCCCGTGTGGAACAATCCCCGGCCAGTCTGCACAGCCCCCTGTGGGGGACAGTATGT
GGGTTCGGACGGAGTGGTCTTGTCCCCCAACTACCCCCAGAACTACACCAGTGGACAGATCTGCTTGT
ATTTTGTTACTGTGCCCAAGGACTATGTGGTGTTTGGCCAGTTCGCCTTCTTTCACACGGCCCTCAAC
GACGTGGTGGAGGTTCACGACGGCCACAGCCAGCACTCGCGGCTCCTCAGCTCCCTCTCGGGCTCCCA
TACAGGAGAATCACTGCCCTTGGCCACCTCCAATCAAGTTCTCATTAAGTTCAGCGCCAAAGGCCTCG
CACCAGCCAGAGGCTTCCACTTTGTCTACCAAGCGGTTCCTCGAACCAGCGCCACGCAGTGCAGCTCT
GTGCCGGAACCCCGCTATGGCAAGAGGCTGGGCAGTGACTTCTCGGTGGGGGCCATCGTCCGCTTCGA
ATGCAACTCCGGCTATGCCCTGCAGGGGTCGCCAGAGATCGAGTGCCTCCCTGTGCCTGGGGCCTTGG
CCCAATGGAATGTCTCAGCGCCCACGTGTGTGGTGCCGTGTGGAGGCAACCTCACAGAGCGCAGGGGC
ACCATCCTGTCCCCTGGCTTCCCAGAGCCGTACCTCAACAGCCTCAACTGTGTGTGGAAGATCGTGGT
CCCCGAAGGCGCTGGCATCCAGATCCAAGTTGTCAGTTTTGTGACAGAGCAGAACTGGGACTCGCTGG
AAGTATTTGATGGTGCAGATAACACTGTAACCATGCTGGGGAGTTTCTCAGGAACAACCGTGCCTGCC
CTTCTGAACAGCACCTCCAACCAGCTCTACCTTCATTTCTACTCAGATATCAGCGTATCTGCAGCTGG
CTTCCACTTGGAGTACAAAACGGTGGGCCTGAGCAGTTGTCCGGAACCTGCTGTGCCCAGTAACGGGG
TGAAGACTGGCGAGCGCTACTTGGTGAATGATGTGGTGTCTTTCCAGTGTGAGCCGGGATATGCCCTC
CAGGGCCACGCCCACATCTCCTGCATGCCCGGAACAGTGCGGCGATGGAACTACCCTCCTCCACTCTG
TATTGCACAGTGTGGGGGAACAGTGGAGGAGATGGAGGGGGTGATCCTGAGCCCCGGCTTCCCAGGCA
ACTACCCCAGTAACATGGACTGCTCCTGGAAAATAGCACTGCCCGTGGGCTTTGGAGCTCACATCCAG
TTCCTGAACTTCTCCACCGAGCCCAACCACGACTACATAGAAATCCGGAATGGCCCCTATGAGACCAG
CCGCATGATGGGAAGATTCAGTGGAAGCGAGCTTCCAAGCTCCCTCCTCTCCACGTCCCACGAGACCA
CCGTGTATTTCCACAGCGACCACTCCCAGAATCGGCCAGGATTCAAGCTGGAGTATCAGGCCTATGAA
CTTCAAGAGTGCCCAGACCCAGAGCCCTTTGCCAATGGCATTGTGAGGGGAGCTGGCTACAACGTGGG
ACAATCAGTGACCTTCGAGTGCCTCCCGGGGTATCAATTGACTGGCCACCCTGTCCTCACGTGTCAAC
ATGGCACCAACCGGAACTGGGACCACCCCCTGCCCAAGTGTGAAGTCCCTTGTGGCGGGAACATCACT
TCTTCCAACGGCACTGTGTACTCCCCGGGGTTCCCTAGCCCGTACTCCAGCTCCCAGGACTGTGTCTG
GCTGATCACCGTGCCCATTGGCCATGGCGTCCGCCTCAACCTCAGCCTGCTGCAGACAGAGCCCTCTG
GAGATTTCATCACCATCTGGGATGGGCCACAGCAAACAGCACCACGGCTCGGCGTCTTCACCCGGAGC
ATGGCCAAGAAAACAGTGCAGAGTTCATCCAACCAGGTCCTGCTCAAGTTCCACCGTGATGCAGCCAC
AGGGGGGATCTTCGCCATAGCTTTCTCCGCTTATCCACTCACCAAATGCCCTCCTCCCACCATCCTCC
CCAACGCCGAAGTCGTCACAGAGAATGAAGAATTCAATATAGGTGACATCGTACGCTACAGATGCCTC
CCTGGCTTTACCTTAGTGGGGAATGAAATTCTGACCTGCAAACTTGGAACCTACCTGCAGTTTGAAGG
ACCACCCCCGATATGTGAAGTGCACTGTCCAACAAATGAGCTTCTGACAGACTCCACAGGCGTGATCC
TGAGCCAGAGCTACCCTGGAAGCTATCCCCAGTTCCAGACCTGCTCTTGGCTGGTGAGAGTGGAGCCC
GACTATAACATCTCCCTCACAGTGGAGTACTTCCTCAGCGAGAAGCAATATGATGAGTTTGAGATTTT
TGATGGTCCATCAGGACAGAGTCCTCTGCTGAAAGCCCTCAGTGGGAATTACTCAGCTCCCCTGATTG
TCACCAGCTCAAGCAACTCTGTGTACCTGCGTTGGTCATCTGATCACGCCTACAATCGGAAGGGCTTC
AAGATCCGCTATTCAGCTCTTTCCTGTGGGCTTCCTGAGGCCCCCAAGAATGGAATGGTGTTTGGCAA
GGAGTACACAGTGGGAACCAAGGCCATGTACAGCTGCAGTGAAGGCTACCACCTCCAGGCAGGCGCTG
AGGCCACTGCAGAGTGTCTGGACACAGGCCTATGGAGCAACCGCAATGTCCCACCACAGTGTGTCCCT
GTGACTTGTCCTGATGTCAGTAGCATCAGCGTGGAGCATGGCCGATGGAGGCTTATCTTTGAGACACA
GTATCAGTTCCAGGCCCAGCTGATGCTCATCTGTGACCCTGGCTACTACTATACTGGCCAAAGGGTCA
TCCGCTGTCAGGCCAATGGCAAATGGAGCCTCGGGGACTCTACGCCCACCTGCCGAATCATCTCCTGT
GGAGAGCTCCCGATTCCCCCCAATGGCCACCGCATCGGAACACTGTCTGTCTACGGGGCAACAGCCAT
CTTCTCCTGCAATTCCGGATACACACTGGTGGGCTCCAGGGTGCGTGAGTGCATGGCCAATGGGCTCT
GGAGTGGCTCTGAAGTCCGCTGCCTTGCTGGACACTGTGGGACTCCTGAGCCCATTGTCAACGGACAC
ATCAATGGGGAGAACTACAGCTACCGGGGCAGTGTGGTGTACCAATGCAATGCTGGCTTCCGCCTGAT
CGGCATGTCTGTGCGCATCTGCCAGCAGGATCATCACTGGTCGGGCAAGACCCCTTTCTGTCAATTAC
CTGTGGACACCCAGGCAACCCTGTCAACGGCCTCACTCAGGGTAACCAGTTTAACCTCAACGATGTGG
TCAAGTTTGTTTGCAACCCTGGGTATATGGCTGAGGGGGCTGCTAGGTCCCAATGCCTGGCCAGCGGG
CAATGGAGTGACATGCTGCCCACCTGCAGAATCATCAACTGTACAGATCCTGGACACCAAGAAAATAG
TGTTCGTCAGGTCCACGCCAGCGGCCCGCACAGGTTCAGCTTCGGCACCACTGTGTCTTACCGGTGCA
ACCACGGCTTCTACCTCCTGGGCACCCCAGTGCTCAGCTGCCAGGGAGATGGCACATGGGACCGTCCC
CGCCCCCAGTGTCTCTGTAAGTAGTGGTGTCCTGTGGCCATCCGGGCTCCCCGCCTCACTCCCAGATG
TCTGGAGACAGTTATACTGTGGGAGCAGTGGTGCGGTACAGCTGCATCGGCAAGCGTACTCTGGTGGG
AAACAGCACCCGCATGTGTGGGCTGGATGGACACTGGACTGGCTCCCTCCCTCACTGCTCAGGAACCA
GCGTGGGAGTTTGCGGTGACCCTGGGATCCCGGCTCATGGCATCCGTTTGGGGGACAGCTTTGATCCA
GGCACTGTGATGCGCTTCAGCTGTGAAGCTGGCCACGTGCTCCGGGGATCGTCAGAGCGCACCTGTCA
AGCCAATGGCTCGTGGAGCGGCTCGCAGCCTGAGTGTGGAGTGATCTCTTGTGGGAACCCTGGGACTC
CAAGTAATGCCCGAGTTGTGTTCAGTGATGGCCTGGTTTTCTCCAGCTCTATCGTCTATGAGTGCCGQ
GAAGGATACTACGCCACAGGCCTGCTCAGCCGTCACTGCTCGGTCAATGGTACCTGGACAGGCAGTGA
CCCTGAGTGCCTCGTCATAAACTGTGGTGACCCTGGGATTCCAGCCAATGGCCTTCGGCTGGGCAATG
ACTTCAGGTACAACAAAACTGTGACATATCAGTGTGTCCCTGGCTATATGATGGAGTCACATAGAGTA
TCTGTGCTGAGCTGCACCAAGGACCGGACATGGAATGGAACCAAGCCCGTCTGCAAAGCTCTCATGTG
CAAGCCACCTCCGCTCATCCCCAATGGGAAGGTGGTGGGGTCTGACTTCATGTGGGGCTCAAGTGTGA
CTTATGCCTGCCTGGAGGGGTACCAGCTCTCCCTGCCCGCGGTGTTCACCTGTGAGGGAAATGGGTCC
TGGACCGGAGAGCTGCCTCAGTGTTTCCCTGTGTTCTGCGGGGATCCTGGTGTCCCGTCCCGTGGGAG
GAGAGAGGACCGAGGCTTCTCCTACAGGTCATCTGTCTCCTTCTCCTGCCATCCCCCTCTGGTGCTGG
TGGGCTCTCCACGCAGGTTTTGCCAGTCAGATGGGACATGGAGTGGCACCCAGCCCAGCTGCATAGAT
CCGACCCTGACCACGTGTGCGGACCCTGGTGTGCCACAGTTTGGGATACAGAACAATTCTCAGGGCTA
CCAGGTTGGAAGCACAGTCCTCTTCCGTTGTCAAAAAGGCTACCTGCTTCAGGGCTCCACCACCAGGA
CCTGCCTCCCAAACCTGACCTGGAGTGGAACCCCACCTGACTGTGTCCCCCACCACTGCAGGCAGCCA
GAGACGCCAACGCATGCCAACGTCGGGGCCCTGGATTTGCCCTCCATGGGCTACACGCTCATTACTCC
TGCCAGGAGGGCTTCTCCCTCAAGGGTGGCTCCGAGCACCGCACCTGCAAGGCGGATGGCAGCTGGAC
AGGCAAGCCGCCCATCTGCCTGGAGGTCCGGCCCAGTGGGAGACCCATCAACACTGCCCGGGAGCCAC
CGCTCACCCAAGCCTTGATTCCTGGGGATGTTTTTGCCAAGAATTCCCTGTGGAAAGGGGCCTATGAA
TACCAGGGGAAGAAGCAGCCAGCCATGCTCAGAGTGACTGGCTTCCAAGTTGCCAACAGCAAGGTCAA
TGCCACCATGATCGACCACAGTGGCGTGGAGCTGCACTTGGCTGGAACTTACAAGAAAGAAGATTTTC
ATCTCCTACTCCAGGTGTACCAGATTACAGGGCCTGTGGAGATCTTTATGAATAAGTTCAAAGATGAT
CACTGGGCTTTAGATGGCCATGTCTCGTCAGAGTCCTCCGGAGCCACCTTCATCTACCAAGGCTCTGT
CAAGGGCCAAGGCTTTGGGCAGTTCGGCTTTCAAAGACTGGACCTCAGGCTGCTGGAGTCAGACCCCG
AGTCCATTGGCCGCCACTTTGCTTCCAACAGCAGCTCAGTGGCAGCCGCGATCCTGGTGCCTTTCATC
GCCCTCATTATTGCGGGCTTCGTGCTCTATCTCTACAAGCACAGGAGAAGACCCAAAGTTCCTTTCAA
TGGCTATGCTGGCCACGAGAACACCAATGTTCGGGCCACATTTGAGAACCCAATGTACGACCGCAACA
TCCAGCCCACAGACATCATGGCCAGCGAGGCGGAGTTCACAGTCAGCACAGTGTGCACAGCAGTATAG
CCACCCGGCCTGGCCGCTTTTTTTGCTAGGTTGAACTGGTACTCCAGCAGCCGCCGAAGCTGGACTGT
ACTGCTGCCATCTCAGCTCACTGCAACCTCCCTGCCTGATTCCCCTGCCTCAGCCTGCCGAGTGCCTG
CGATTGCAGGCGCGCACCGCCAC

| | | | |
|---|---|---|---|
| NOV4d, CG50377-03 | SEQ ID NO: 38 | 2612 aa | MW at 284047.5kD |
| Protein Sequence | | | |

MAGAPPPALLLPCSLISDCCASNQRHSVGVGPSELVKKQIELKSRGVKLMPSKDNSQKTSVLTQVGVS
QGHNMCPDPGIPERGKRLGSDFRLGSSVQFTCNEGYDLQGSKRITCMKVSDMFAAWSDHRPVCRARMC
DAHLRGPSGIITSPNFPIQYDNNAHCVWIITALNPSKVIKLAFEEFDLERGYDTLTVGDGGQDGDQKT
VLYMSQNACSDSPHTPGSRIPESMSGDIWRQKWTVLEICRDISSSDARSGSVRKSPKTSNAVELVAPG
TEIEQGSCGDPGIPAYGRREGSRFHHGDTLKFECQPAFELVGQKAITCQKNNQWSAKKPGCVFSCFFN
FTSPSGVVLSPNYPEDYGNHLHCVWLILARPESRIHLAFNDIDVEPQFDFLVIKDGATAEAPVLGTFS
GNQLPSSITSSGHVARLEFQTDHSTGKRGFNITFTTFRHNECPDPGVPVNGKRFGDSLQLGSSISFLC
DEGFLGTQGSETITCVLKEGSVVWNSAVLRCEAPCGGHLTSPSGTILSPGWPGFYKDALSCAWVIEAQ
PGYPIKITFDRFKTEVNYDTLEVRDGRTYSAPLIGVYHGTQVPQFLISTSNYLYLLFSTDKSHSDIGF
QLRYETITLQSDHCLDPGIPVNGQRHGNDFYVGALVTFSCDSGYTLSDGEPLECEPNFQWSRALPSCE
ALCGGFIQGSSGTTLSPGFPDFYPNNLNCTWIIETSHGKGVFFTFHTFHLESGHDYLLITENGSFTQP
LRQLTGSRLPAPISAGLYGNFTAQVRFISDFSMSYEGFNITFSEYDLEPCEEPEVPAYSIRKGLQFGV
GDTLTFSCFPGYRLEGTARITCLGGRRRLWSSPLPRCVAECGNSVTGTQGTLLSPNFPVNYNNNHECI
YSIQTQPGKGIQLKARAFELSEGDVLKVYDGNNNSARLLGVFSHSEMMGVTLNSTSSSLWLDFITDAE
NTSKGFELHFSSFELIKCEDPGTPKFGYKVHDEGHFAGSSVSFSCDPGYSLRGSEELLCLSGERRTWD
RPLPTCVAECGGTVRGEVSGQVLSPGYPAPYEHNLNCIWTIEAEAGCTIGLHFLVFDTEEVHDVLRIW
DGPVESGVLLKELSGPALPKDLHSTFNSVVLQFSTDFFTSKQGFAIQFSVSTATSCNDPGIPQNGSRS
GDSWEAGDSTVFQCDPGYALQGSAEISCVKIENRFFWQPSPPTCIAPCGGDLTGPSGVILSPNYPEPY
PPGKECDWKVTVSPDYVIALVFNIFNLEPGYDFLHIYDGRDSLSPLIGSFYGSQLPGRIESSSNSLFL
AFRSDASVSNAGFVIDYTENPRESCFDPGSIKNGTRVGSDLKLGSSVTYYCHGGYEVEGTSTLSCILG
PDGKPVWNNPRPVCTAPCGGQYVGSDGVVLSPNYPQNYTSGQICLYFVTVPKDYVVFGQFAFFHTALN
DVVEVHDGHSQHSRLLSSLSGSHTGESLPLATSNQVLIKFSAKGLAPARGFHFVYQAVPRTSATQCSS
VPEPRYGKRLGSDFSVGAIVRFECNSGYALQGSPEIECLPVPGALAQWNVSAPTCVVPCGGNLTERRG
TILSPGFPEPYLNSLNCVWKIVVPEGAGIQIQVVSFVTEQNWDSLEVFDGADNTVTMLGSFSGTTVPA
LLNSTSNQLYLHFYSDISVSAAGFHLEYKTVGLSSCPEPAVPSNGVKTGERYLVNDVVSFQCEPGYAL
QGHAHISCMPGTVRRWNYPPPLCIAQCGGTVEEMEGVILSPGFPGNYPSNMDCSWKIALPVGFGAHIQ
FLNFSTEPNHDYIEIRNGPYETSRMMGRFSGSELPSSLLSTSHETTVYFHSDHSQNRPGFKLEYQAYE
LQECPDPEPFANGIVRGAGYNVGQSVTFECLPGYQLTGHPVLTCQHGTNRNWDHPLPKCEVPCGGNIT
SSNGTVYSPGFPSPYSSSQDCVWLITVPIGHGVRLNLSLLQTEPSGDFITIWDGPQQTAPRLGVFTRS
MAKKTVQSSSNQVLLKFHRDAATGGIFAIAFSAYPLTKCPPPTILPNAEVVTENEEFNIGDIVRYRCL
PGFTLVGNEILTCKLGTYLQFEGPPPICEVHCPTNELLTDSTGVILSQSYPGSYPQFQTCSWLVRVEP
DYNISLTVEYFLSEKQYDEFEIFDGPSGQSPLLKALSGNYSAPLIVTSSSNSVVLRWSSDHAYNRKGF
KIRYSALSCGLPEAPKNGMVFGKEYTVGTKAMYSCSEGYHLQAGAEATAECLDTGLWSNRNVPPQCVP
VTCPDVSSISVEHGRWRLIFETQYQFQAQLMLICDPGYYYTGQRVIRCQANGKWSLGDSTPTCRIISC
GELPIPPNGHRIGTLSVYGATAIFSCNSGYTLVGSRVRECMANGLWSGSEVRCLAGHCGTPEPIVNGH
INGENYSYRGSVVYQCNAGFRLIGMSVRICQQDHHWSGKTPFCQLPVDTQATLSTASLRVTSLTSTMW
SSLFATLGIWLRGLLGPNAWPAGNGVTCCPPAESSTVQILDTKKIVFVRSTPAARTGSASAPLCLTGA
TTASTSWAPQCSAAREMAHGTVPAPSVSVSSGVLWPSGLPASLPDVWRQLYCGSSGAVQLHRQAYSGG
KQHPHVWAGWTLDWLPPSLLPNQRGSLR

| | | |
|---|---|---|
| NOV4e, CG50377-05 | SEQ ID NO: 39 | 10466 bp |
| DNA Sequence | ORF Start: at 1 | ORF Stop: TAG at 10450 |

GGCCAGAACTGCACGTTCCAACTGCACGGTCCCAATGGGACAGTTGAGAGCCCAGGGTTCCCATATGG
CTACCCCAATTACGCCAACTGCACGTGGACCATCACCGCGGAAGAGCAGCACAGAATCCAGCTTGTGT
TCCAGTCCTTTGCCCTGGAAGAGGACTTTGATGTCCTGTCGGTGTTTGATGGTCCACCCCAGCCAGAG
AATCTGCGTACGAGGCTCACAGGCTTTCAGCTGCCAGCCACCATTGTTAGTGCAGCCACCACCCTCTC
TCTGCGCCTCATCAGCGACTATGCAGTCAGTGCCCAAGGCTTCCACGCCACCTATGAAGTTCTCCCCA
GCCACACATGTGGGAACCCAGGGAGGCTGCCCAATGGCATCCAGCAGGGTTCAACCTTCAACCTCGGT
GACAAGGTCCGCTACAGCTGCAACCTTGGCTTCTTCCTGGAGGGCCACGCCGTGCTCACCTGCCACGC
TGGCTCTGAGAACAGCGCCACGTGGGACTTCCCCCTGCCTTCCTGCAGAGCTGATGATGCCTGTGGTG
GGACCCTGCGGGGCCAGAGTGGCATCATCTCCAGCCCCCACTTCCCCTCGGAGTACCATAACAATGCC
GACTGCACATGGACCATCCTGGCTGAGCTGGGGGACACCATCGCCCTGGTGTTTATTGACTTCCAGCT
GGAGGATGGTTACGACTTTCTGGAAGTCACTGGGACAGAAGGCTCCTCCCTCTGGTTCACCGGAGCCA
GCCTCCCAGCCCCCGTTATCAGCAGCAAGAACTGGCTGCGACTGCACTTCACATCGGATGGCAACCAC
CGGCAGCGCGGATTCAGTGCCCAATACCAAGTCAAGAAGCAAATTGAGTTGAAGTCTCGAGGTGTGAA
GCTGATGCCCAGCAAAGACAACAGCCAGAAGACGTCTGTGGTAACTCAGGTTGGTGTGTCCCAAGGAC
ATAATATGTGTCCAGACCCTGGCATACCCGAAAGGGGCAAAAGACTAGGCTCGGATTTCAGGTTAGGA
TCCAGCGTCCAGTTCACCTGCAACGAGGGCTATGACCTGCAAGGGTCCAAGCGGATCACCTGTATGAA
AGTGAGCGACATGTTTGCGGCCTGGAGCGACCACAGGCCAGTCTGCCGAGCCCGCATGTGTGATGCCC
ACCTTCGAGGCCCCTCGGGCATCATCACCTCCCCCAATTTCCCCATTCAGTATGACAACAATGCACAC
TGTGTGTGGATCATCACAGCACTCAACCCCTCCAAGGTAATCAAGCTCGCCTTTGAGGAGTTTGATTT
GGAGAGGGGCTATGACACCCTGACGGTCGGTGATGGTGGTCAGGATGGGGACCAGAAGACAGTTCTCT
ACATGCTGACAGGTACATCGGTCCCGGATCTCATTGTCAGCACCAATCATCAAATGTGGCTCCTCTTC
CAGACTGATGGCAGTGGCAGTTCCCTGGGATTCAAGGCTTCTTATGAAGAGATCGAGCAGGGCAGTTG
CGGTGACCCTGGCATACCTGCATATGGCCGGAGGGAAGGCTCCCGGTTTCGCCACGGTGACACACTCA
AGTTTGAGTGCCAGCCCGCCTTTGAGCTGGTGGGACAGAAGGCAATCACATGCCAAAAGAATAACCAA
TGGTCGGCTAAGAAGCCAGGCTGCGTGTGTTCCTGCTTCTTCAACTTCACCAGCCCGTCTGGGGTTGT
CCTGTCTCCCAACTACCCAGAGGACTATGGCAACCACCTCCACTGTGTCTGGCTCATCCTGGCCAGGC
CTGAGAGCCGCATCCACCTGGCCTTCAACGACATTGACGTGGAGCCTCAGTTTGATTTCCTGGTCATC
AAGGATGGGGCCACCGCCGAGGCGCCCGTCCTGGGCACCTTCTCAGGAAACCAGCTTCCCTCCTCCAT
CACAAGCAGTGGCCACGTGGCCCGTCTCGAGTTCCAGACTGACCACTCCACAGGGAAGAGGGGCTTCA
ACATCACTTTTACCAGTGAGTCCTCCAACGAGTGCCCGGATCCTGGCGTTCCAGTAAATGGCAAACGG
TTTGGGGACAGCCTCCAGCTGGGCAGCTCCATCTCCTTCCTCTGTGATGAAGGCTTCCTTGGGACTCA
GGGCTCAGAGACCATCACCTGCGTCCTGAAGGAGGGCAGCGTGGTCTGGAACAGCGCTGTGCTGCGGT
GTGAAGCTCCCTGTGGTGGTCACCTGACTTCGCCCAGCGGCACCATCCTCTCTCCGGGCTGGCCTGGC
TTCTACAAGGATGCCTTGAGCTGTGCCTGGGTGATTGAGGCCCAGCCAGGCTACCCCATCAAAATCAC
CTTCGACAGGTTCAAAACCGAGGTCAACTATGACACCCTGGAAGTACGCGATGGGCGGACTTACTCAG
CGCCCTTGATCGGGGTTTACCACGGGACCCAGGTTCCCCAGTTCCTCATCAGCACCAGCAACTACCTC
TACCTCCTCTTCTCTACCGACAAGAGTCACTCGGACATCGGCTTCCAGCTCCGCTATGAGGCTATAAC
ACTGCAGTCAGACCACTGTCTGGATCCAGGAATCCCAGTAAATGGACAGCGTCATGGGAATGACTTCT
ACGTGGGCGCGCTGGTGACCTTCAGCTGTGACTCGGGCTACACATTAAGTGACGGGGAGCCTCTGGAG
TGTGAGCCCAACTTCCAGTGGAGCCGGGCCCTGCCCAGTTGTGAAGCTCTCTGTGGTGGCTTCATTCA
AGGCTCCAGTGGGACCATCTTGTCGCCAGGTTTCCCTGACTTCTACCCCAACAACTTGAACTGCACCT
GGATTATCGAAACATCTCATGGCAAGGGTGTGTTCTTCACTTTCCACACCTTCCACCTGGAAAGTGGC
CATGACTACCTCCTCATCACTGAGAACGGCAGCTTCACCCAGCCCCTGAGGCAGCTAACTGGATCTCG
GCTGCCAGCTCCCATCAGCGCTGGGCTCTATGGCAACTTCACTGCCCAGGTCCGCTTCATCTCTGATT
TCTCCATGTCATATGAAGGATTCAACATCACCTTCTCAGAGTACGACTTGGAGCCCTGTGAGGAGCCC
GAGGTCCCAGCCTACAGCATCCGGAAGGGCTTGCAGTTTGGCGTGGGCGACACCTTGACCTTCTCCTG
CTTCCCCGGGTACCGTCTGGAGGGCACCGCCCGCATCACGTGCCTGGGGGGCAGACGGCGCCTGTGGA
GCTCGCCTCTGCCAAGGTGTGTTGCTGAGTGTGGGAATTCAGTCACAGGCACTCAGGGTACTTTGCTG
TCCCCCAACTTTCCTGTGAACTACAATAACAATCATGAATGCATCTACTCCATCCAGACCCAGCCAGG
GAAGGGAATTCAGCTGAAAGCCAGGGCATTCGAACTCTCCGAAGGAGATGTCCTCAAGGTTTATGATG
GCAACAACAACTCCGCCCGTTTGCTGGGAGTTTTTAGCCATTCTGAGATGATGGGGGTGACTTTGAAC
AGCACATCCAGCAGTCTGTGGCTTGATTTCATCACTGATGCTGAAAACACCAGCAAGGGCTTTGAACT
GCACTTTTCCAGTTTTGAACTCATCAAATGTGAGGACCCAGGAACCCCCAAGTTTGGCTACAAGGTTC
ATGATGAAGGTCATTTTGCAGGGAGCTCCGTGTCCTTCAGCTGTGACCCTGGATACAGCCTGCGGGGT
AGTGAGGAGCTGCTGTGTCTGAGTGGAGAGCGCCGGACCTGGGACCGGCCTCTGCCCACCTGTGTCGC
CGAGTGTGGAGGGACAGTGAGAGGAGAGGTGTCGGGGCAGGTGCTGTCACCCGGGTATCCAGCTCCCT
ATGAACACAATCTCAACTGCATCTGGACCATCGAAGCAGAGGCCGGCTGCACCATTGGGCTACACTTC
CTGGTGTTTGACACAGAGGAGGTTCACGACGTGCTGCGCATCTGGGATGGGCCTGTGGAGAGCGGGGT
TCTGCTGAAGGAGCTGAGTGGCCCGGCCCTGCCCAAGGACCTGCATAGCACCTTCAACTCGGTCGTCC
TGCAGTTCAGCACTGACTTCTTCACCAGCAAGCAGGGCTTTGCCATTCAATTTTCAGGTTCCACAGCA
ACGTCCTGCAATGACCCTGGGATCCCGCAGAATGGGAGTCGGAGTGGTGACAGTTGGGAAGCCGGCGA
CTCCACAGTGTTCCAGTGTGACCCTGGCTACGCGCTGCAGGGAAGTGCAGAGATCAGCTGTGTGAAGA
TCGAGAACAGGTTCTTCTGGCAGCCCAGCCCGCCAACATGCATCGCTCCCTGCGGGGGAGACCTGACA
GGACCATCTGGAGTCATCCTCTCACCAAATTACCCAGAACCCTACCCGCCAGGCAAGGAGTGTGACTG
GAAAGTGACCGTCTCACCAGACTACGTCATCGCCCTGGTATTGTTCCCCAGCTTTAACCTGGAGCCTG
GCTATGACTTCCTCCATATCTACGACGGACGGGACTCTCTCAGCCCTCTCATAGGAAGCTTCTATGGC
TCCCAGCTCCCAGGCCGCATTGAAAGCAGCAGCAACAGCCTCTTCCTCGCCTTCCGCAGCGATGCATC
TGTGAGCAATGCTGGCTTCGTCATTGACTTCCCAGAAAACCCGCGGGAGTCATGTTTTGATCCTGGTT
CCATCAAGAACGGCACACGGGTGGGGTCCGACCTGAAGCTGGGCTCCTCCGTCACCTACTACTGCCAC
GGGGGCTACGAAGTTGAGGGCACCTCGACCCTGAGCTGCATCCTGGGGCCTGATGGGAAGCCCGTGTG
GAACAATCCCCGGCCAGTCTGCACAGCCCCCTGTGGGGGACAGTATGTGGGTTCGGACGGAGTGGTCT
TGTCCCCCAACTACCCCCAGAACTACACCAGTGGACAGATCTGCTTGTATTTTGTTACTGTGCCCAAG
GACTATGGTGTGGTGTTTGGCCAGTTCGCCTTCTTTCACACGGCCCTCAACGACGTGGTGGAGGTTCA
CGACGGCCACAGCCAGCACTCGCGGCTCCTCAGCTCCCTCTCGGGCTCCCATACAGGTGAATCACTGC
CCTTGGCCACCTCCAATCAAGTTCTCATTAAGTTCAGCGCCAAAGGCCTCGCACCAGCCAGAGGCTTC
CACTTTGTCTACCAAGCGGTTCCTCGAACCAGCGCCACGCAGTGCAGCTCTGTGCCGGAACCCCGCTA
TGGCAAGAGGCTGGGCAGTGACTTCTCGGTGGGGGCCATCGTCCGCTTCGAATGCAACTCCGGCTATG
CCCTGCAGGGGTCGCCAGAGATCGAGTGCCTCCCTGTGCCTGGGGCCTTGGCCCAATGGAATGTCTCA
GCGCCCACGTGTGTGGTGCCGTGTGGAGGCAACCTCACAGAGCGCAGGGGCACCATCCTGTCCCCTGG
CTTCCCAGAGCCGTACCTCAACAGCCTCAACTGTGTGTGGAAGATCGTGGTCCCCGAAGGCGCTGGCA
TCCAGATCCAAGTTGTCAGTTTTGTGACAGAGCAGAACTGGGACTCGCTGGAAGTATTTGATGGTGCA
GATAACACTGTAACCATGCTGGGGAGTTTCTCAGGAACAACCGTGCCTGCCCTTCTGAACAGCACCTC
CAACCAGCTCTACCTTCATTTCTACTCAGATATCAGCGTATCTGCAGCTGGCTTCCACTTGGAGTACA
AAACGGTGGGCCTGAGCAGTTGTCCGGAACCTGCTGTGCCCAGTAACGGGGTGAAGACTGGCGAGCGC
TACTTGGTGAATGATGTGGTGTCTTTCCAGTGTGAGCCGGGATATGCCCTCCAGGGCCACGCCCACAT
CTCCTGCATGCCCGGAACAGTGCGGCGATGGAACTACCCTCCTCCACTCTGTATTGCACAGTGTGGGG
GAACAGTGGAGGAGATGGAGGGGGTGATCCTGAGCCCCGGCTTCCCAGGCAACTACCCCAGTAACATG
GACTGCTCCTGGAAAATAGCACTGCCCGTGGGCTTTGGAGCTCACATCCAGTTCCTGAACTTCTCCAC
CGAGCCCAACCACGACTACATAGAAATCCGGAATGGCCCCTATGAGACCAGCCGCATGATGGGAAGAT
TCAGTGGAAGCGAGCTTCCAAGCTCCCTCCTCTCCACGTCCCACGAGACCACCGTGTATTTCCACAGC
GACCACTCCCAGAATCGGCCAGGATTCAAGCTGGAGTATCAGGCCTATGAACTTCAAGAGTGCCCAGA
CCCAGAGCCCTTTGCCAATGGCATTGTGAGGGGAGCTGGCTACAACGTGGGACAATCAGTGACCTTCG
AGTGCCTCCCGGGGTATCAATTGACTGGCCACCCTGTCCTCACGTGTCAACATGGCACCAACCGGAAC
TGGGACCACCCCCTGCCCAAGTGTGAAGTCCCTTGTGGCGGGAACATCACTTCTTCCAACGGCACTGT
GTACTCCCCGGGGTTCCCTAGCCCGTACTCCAGCTCCCAGGACTGTGTCTGGCTGATCACCGTGCCCA
TTGGCCATGGCGTCCGCCTCAACCTCAGCCTGCTGCAGACAGAGCCCTCTGGAGATTTCATCACCATC
TGGGATGGGCCACAGCAAACAGCACCACGGCTCGGCGTCTTCACCCGGAGCATGGCCAAGAAAACAGT
GCAGAGTTCATCCAACCAGGTCCTGCTCAAGTTCCACCGTGATGCAGCCACAGGGGGGATCTTCGCCA
TAGCTTTCTCCGCTTATCCACTCACCAAATGCCCTCCTCCCACCATCCTCCCCAACGCCGAAGTCGTC
ACAGAGAATGAAGAATTCAATATAGGTGACATCGTACGCTACAGATGCCTCCCTGGCTTTACCTTAGT
GGGGAATGAAATTCTGACCTGCAAACTTGGAACCTACCTGCAGTTTGAAGGACCACCCCCGATATGTG
AAGTGCACTGTCCAACAAATGAGCTTCTGACAGACTCCACAGGCGTGATCCTGAGCCAGAGCTACCCT
GGAAGCTATCCCCAGTTCCAGACCTGCTCTTGGCTGGTGAGAGTGGAGCCCGACTATAACATCTCCCT
CACAGTGGAGTACTTCCTCAGCGAGAAGCAATATGATGAGTTTGAGATTTTTGATGGTCCATCAGGAC
AGAGTCCTCTGCTGAAAGCCCTCAGTGGGAATTACTCAGCTCCCCTGATTGTCACCAGCTCAAGCAAC
TCTGTGTACCTGCGTTGGTCATCTGATCACGCCTACAATCGGAAGGGCTTCAAGATCCGCTATTCAGC
CCTTTCCTGTGGGCTTCCTGAGGCCCCCAAGAATGGAATGGTGTTTGGCAAGGAGTACACAGTGGGAA
CCAAGGCCATGTACAGCTGCAGTGAAGGCTACCACCTCCAGGCAGGCGCTGAGGCCACTGCAGAGTGT
CTGGACACAGGCCTATGGAGCAACCGCAATGTCCCACCACAGTGTGTCCCTGTGACTTGTCCTGATGT
CAGTAGCATCAGCGTGGAGCATGGCCGATGGAGGCTTATCTTTGAGACACAGTATCAGTTCCAGGCCC
AGCTGATGCTCATCTGTGACCCTGGCTACTACTATACTGGCCAAAGGGTCATCCGCTGTCAGGCCAAT
GGCAAATGGAGCCTCGGGGACTCTACGCCCACCTGCCGAATCATCTCCTGTGGAGAGCTCCCGATTCC
CCCCAATGGCCACCGCATCGGAACACTGTCTGTCTACGGGGCAACAGCCATCTTCTCCTGCAATTCCG
GATACACACTGGTGGGCTCCAGGGTGCGTGAGTGCATGGCCAATGGGCTCTGGAGTGGCTCTGAAGTC
CGCTGCCTTGCTGGACACTGTGGGACTCCTGAGCCCATTGTCAACGGACACATCAATGGGGAGAACTA
CAGCTACCGGGGCAGTGTGGTGTACCAATGCAATGCTGGCTTCCGCCTGATCGGCATGTCTGTGCGCA
TCTGCCAGCAGGATCATCACTGGTCGGGCAAGACCCCTTTCTGTGTGCCAATTACCTGTGGACACCCA
GGCAACCCTGTCAACGGCCTCACTCAGGGTAACCAGTTTAACCTCAACGATGTGGTCAAGTTTGTTTG
CAACCCTGGGTATATGGCTGAGGGGGCTGCTAGGTCCCAATGCCTGGCCAGCGGGCAATGGAGTGACA
TGCTGCCCACCTGCAGAATCATCAACTGTACAGATCCTGGACACCAAGAAAATAGTGTTCGTCAGGTC
CACGCCAGCGGCCCGCACAGGTTCAGCTTCGGCACCACTGTGTCTTACCGGTGCAACCACGGCTTCTA
CCTCCTGGGCACCCCAGTGCTCAGCTGCCAGGGAGATGGCACATGGGACCGTCCCCGCCCCCAGTGTC
TCCTGGTGTCCTGTGGCCATCCGGGCTCCCCGCCTCACTCCCAGATGTCTGGAGACAGTTATACTGTG
GGAGCAGTGGTGCGGTACAGCTGCATCGGCAAGCGTACTCTGGTGGGAAACAGCACCCGCATGTGTGG
GCTGGATGGACACTGGACTGGCTCCCTCCCTCACTGCTCAGGAACCAGCGTGGGAGTTTGCGGTGACC
CTGGGATCCCGGCTCATGGCATCCGTTTGGGGGACAGCTTTGATCCAGGCACTGTGATGCGCTTCAGC
TGTGAAGCTGGCCACGTGCTCCGGGGATCGTCAGAGCGCACCTGTCAAGCCAATGGCTCGTGGAGCGG
CTCGCAGCCTGAGTGTGGAGTGATCTCTTGTGGGAACCCTGGGACTCCAAGTAATGCCCGAGTTGTGT
TCAGTGATGGCCTGGTTTTCTCCAGCTCTATCGTCTATGAGTGCCGGGAAGGATACTACGCCACAGGC
CTGCTCAGCCGTCACTGCTCGGTCAATGGTACCTGGACAGGCAGTGACCCTGAGTGCCTCGTCATAAA
CTGTGGTGACCCTGGGATTCCAGCCAATGGCCTTCGGCTGGGCAATGACTTCAGGTACAACAAAACTG
TGACATATCAGTGTGTCCCTGGCTATATGATGGAGTCACATAGAGTATCTGTGCTGAGCTGCACCAAG
GACCGGACATGGAATGGAACCAAGCCCGTCTGCAAAGCTCTCATGTGCAAGCCACCTCCGCTCATCCC
CAATGGGAAGGTGGTGGGGTCTGACTTCATGTGGGGCTCAAGTGTGACTTATGCCTGCCTGGAGGGGT
ACCAGCTCTCCCTGCCCGCGGTGTTCACCTGTGAGGGAAATGGGTCCTGGACCGGAGAGCTGCCTCAG
TGTTTCCCTGTGTTCTGCGGGGATCCTGGTGTCCCGTCCCGTGGGAGGAGAGAGGACCGAGGCTTCTC
CTACAGGTCATCTGTCTCCTTCTCCTGCCATCCCCCTCTGGTGCTGGTGGGCTCTCCACGCAGGTTTT
GCCAGTCAGATGGGACATGGAGTGGCACCCAGCCCAGCTGCATAGATCCGACCCTGACCACGTGTGCG
GACCCTGGTGTGCCACAGTTTGGGATACAGAACAATTCTCAGGGCTACCAGGTTGGAAGCACAGTCCT
CTTCCGTTGTCAAAAAGGCTACCTGCTTCAGGGCTCCACCACCAGGACCTGCCTCCCAAACCTGACCT
GGAGTGGAACCCCACCTGACTGTGTCCCCCACCACTGCAGGCAGCCAGAGACGCCAACGCATGCCAAC
GTCGGGGCCCTGGATTTGCCCTCCATGGGCTACACGCTCATCTACTCCTGCCAGGAGGGCTTCTCCCT
CAAGGGTGGCTCCGAGCACCGCACCTGCAAGGCGGATGGCAGCTGGACAGGCAAGCCGCCCATCTGCC
TGGAGGTCCGGCCCAATGGGAGACCCATCAACACTGCCCGGGAGCCACCGCTCACCCAAGCCTTGATT
CCTGGGGATGTTTTTGCCAAGAATTCCCTGTGGAAAGGGGCCTATGAATACCAGGGGAAGAAGCAGCC
AGCCATGCTCAGAGTGACTGGCTTCCAAGTTGCCAACAGCAAGGTCAATGCCACCATGATCGACCACA
GTGGCGTGGAGCTGCACTTGGCTGGAACTTACAAGAAAGAAGATTTTCATCTCCTACTCCAGGTGTAC
CAGATTACAGGGCCTGTGGAGATCTTTATGAATAAGTTCAAAGATGATCACTGGGCTTTAGATGGCCA
TGTCTCGTCAGAGTCCTCCGGAGCCACCTTCATCTACCAAGGCTCTGTCAAGGGCCAAGGCTTTGGGC
AGTTCGGCTTTCAAAGACTGGACCTCAGGCTGCTGGAGTCAGACCCCGAGTCCATTGGCCGCCACTTT
GCTTCCAACAGCAGCTCAGTGGCAGCCGCGATCCTGGTGCCTTTCATCGCCCTCATTATTGCGGGCTT
CGTGCTCTATCTCTACAAGCACAGGAGAAGACCCAAAGTTCCTTTCAATGGCTATGCTGGCCACGAGA
ACACCAATGTTCGGGCCACATTTGAGAACCCAATGTACGACCGCAACATCCAGCCCACAGACATCATG
GCCAGCGAGGCGGAGTTCACAGTCAGCACAGTGTGCACAGCAGTATAGCCACCCGGCCTGGC

| | | | |
|---|---|---|---|
| NOV4e, CG50377-05 | SEQ ID NO: 40 | 3483 aa | MW at 379077.1kD |
| Protein Sequence | | | |

GQNCTFQLHGPNGTVESPGFPYGYPNYANCTWTITAEEQHRIQLVFQSFALEEDFDVLSVFDGPPQPE
NLRTRLTGFQLPATIVSAATTLSLRLISDYAVSAQGFHATYEVLPSHTCGNPGRLPNGIQQGSTFNLG
DKVRYSCNLGFFLEGHAVLTCHAGSENSATWDFPLPSCRADDACGGTLRGQSGIISSPHFPSEYHNNA
DCTWTILAELGDTIALVFIDFQLEDGYDFLEVTGTEGSSLWFTGASLPAPVISSKNWLRLHFTSDGNH
RQRGFSAQYQVKKQIELKSRGVKLMPSKDNSQKTSWTQVGVSQGHNMCPDPGIPERGKREJGSDFRLG
SSVQFTCNEGYDLQGSKRITCMKVSDMFAAWSDHRPVCEARMCDAHLRGPSGIITSPNFPIQYDNNAH
CVWIITALNPSKVIKLAFEEFDLERGYDTLTVGDGGQDGDQKTVLYMLTGTSVPDLIVSTNHQMWLLF
QTDGSGSSLGFKASYEEIEQGSCGDPGIPAYGRREGSRFRHGDTLKFECQPAFELVGQKAITCQKNNQ
WSAKKPGCVCSCFFNFTSPSGVVLSPNYPEDYGNHLHCVWLILARPESRIHLAFNDIDVEPQFDFLVI
KDGATAEAPVLGTFSGNQLPSSITSSGHVARLEFQTDHSTGKRGFNITFTSESSNECPDPGVPVNGKR
FGDSLQLGSSISFLCDEGFLGTQGSETITCVLKEGSVVWNSAVLRCEAPCGGHLTSPSGTILSPGWPG
FYKDALSCAWVIEAQPGYPIKITFDRFKTEVNYDTLEVRDGRTYSAPLIGVYHGTQVPQFLISTSNYL
YLLFSTDKSHSDIGFQLRYEAITLQSDHCLDPGIPVNGQRHGNDFYVGALVTFSCDSGYTLSDGEPLE
CEPNFQWSRALPSCEALCGGFIQGSSGTILSPGFPDFYPNNLNCTWIIETSHGKGVFFTFHTFHLESG
HDYLLITENGSFTQPLRQLTGSRLPAPISAGLYGNFTAQVRFISDFSMSYEGFNITFSEYDLEPCEEP
EVPAYSIRKGLQFGVGDTLTFSCFPGYRLEGTARITCLGGRRRLWSSPLPRCVAECGNSVTGTQGTLL
SPNFPVNYNNNHECIYSIQTQPGKGIQLKARAFELSEGDVLKVYDGNNNSARLLGVFSHSEMMGVTLN
STSSSLWLDFITDAENTSKGFELHFSSFELIKCEDPGTPKFGYKVHDEGHFAGSSVSFSCDPGYSLRG
SEELLCLSGERRTWDRPLPTCVAECGGTVRGEVSGQVLSPGYPAPYEHNLNCIWTIEAEAGCTIGLHF
LVFDTEEVHDVLRIWDGPVESGVLLKELSGPALPKDLHSTFNSVVLQFSTDFFTSKQGFAIQFSGSTA
TSCNDPGIPQNGSRSGDSWEAGDSTVFQCDPGYALQGSAEISCVKIENRFFWQPSPPTCIAPCGGDLT
GPSGVILSPNYPEPYPPGKECDWKVTVSPDYVIALVLFPSFNLEPGYDFLHIYDGRDSLSPLIGSFYG
SQLPGRIESSSNSLFLAFRSDASVSNAGFVIDFPENPRESCFDPGSIKNGTRVGSDLKLGSSVTYYCH
GGYEVEGTSTLSCILGPDGKPVWNNPRPVCTAPCGGQYVGSDGVVLSPNYPQNYTSGQICLYFVTVPK
DYGVVFGQFAFFHTALNDVVEVHDGHSQHSRLLSSLSGSHTGESLPLATSNQVLIKFSAKGLAPARGF
HFVYQAVPRTSATQCSSVPEPRYGKRLGSDFSVGAIVRFECNSGYALQGSPEIECLPVPGALAQWNVS
APTCVVPCGGNLTERRGTILSPGFPEPYLNSLNCVWKIVVPEGAGIQIQVVSFVTEQNWDSLEVFDGA
DNTVTMLGSFSGTTVPALLNSTSNQLYLHFYSDISVSAAGFHLEYKTVGLSSCPEPAVPSNGVKTGER
YLVNDVVSFQCEPGYALQGHAHISCMPGTVRRWNYPPPLCIAQCGGTVEEMEGVILSPGFPGNYPSNM
DCSWKIALPVGFGAHIQFLNFSTEPNHDYIEIRNGPYETSRMMGRFSGSELPSSLLSTSHETTVYFHS
DHSQNRPGFKLEYQAYELQECPDPEPFANGIVRGAGYNVGQSVTFECLPGYQLTGHPVLTCQHGTNRN
WDHPLPKCEVPCGGNITSSNGTVYSPGFPSPYSSSQDCVWLITVPIGHGVRLNLSLLQTEPSGDFITI
WDGPQQTAPRLGVFTRSMAKKTVQSSSNQVLLKFHRDAATGGIFAIAFSAYPLTKCPPPTILPNAEVV
TENEEFNIGDIVRYRCLPGFTLVGNEILTCKLGTYLQFEGPPPICEVHCPTNELLTDSTGVILSQSYP
GSYPQFQTCSWLVRVEPDYNISLTVEYFLSEKQYDEFEIFDGPSGQSPLLKALSGNYSAPLIVTSSSN
SVYLRWSSDHAYNRKGFKIRYSALSCGLPEAPKNGMVFGKEYTVGTKAMYSCSEGYHLQAGAEATAEC
LDTGLWSNRNVPPQCVPVTCPDVSSISVEHGRWRLIFETQYQFQAQLMLICDPGYYYTGQRVIRCQAN
GKWSLGDSTPTCRIISCGELPIPPNGHRIGTLSVYGATAIFSCNSGYTLVGSRVRECMANGLWSGSEV
RCLAGHCGTPEPIVNGHINGENYSYRGSVVYQCNAGFRLIGMSVRICQQDHHWSGKTPFCVPITCGHP
GNPVNGLTQGNQFNLNDVVKFVCNPGYMAEGAARSQCLASGQWSDMLPTCRIINCTDPGHQENSVRQV
HASGPHRFSFGTTVSYRCNHGFYLLGTPVLSCQGDGTWDRPRPQCLLVSCGHPGSPPHSQMSGDSYTV
GAVVRYSCIGKRTLVGNSTRMCGLDGHWTGSLPHCSGTSVGVCGDPGIPAHGIRLGDSFDPGTVMRFS
CEAGHVLRGSSERTCQANGSWSGSQPECGVISCGNPGTPSNARVVFSDGLVFSSSIVYECREGYYATG
LLSRHCSVNGTWTGSDPECLVINCGDPGIPANGLRLGNDFRYNKTVTYQCVPGYMMESHRVSVLSCTK
DRTWNGTKPVCKALMCKPPPLIPNGKVVGSDFMWGSSVTYACLEGYQLSLPAVFTCEGNGSWTGELPQ
CFPVFCGDPGVPSRGRREDRGFSYRSSVSFSCHPPLVLVGSPRRFCQSDGTWSGTQPSCIDPTLTTCA
DPGVPQFGIQNNSQGYQVGSTVLFRCQKGYLLQGSTTRTCLPNLTWSGTPPDCVPHHCRQPETPTHAN
VGALDLPSMGYTLIYSCQEGFSLKGGSEHRTCKADGSWTGKPPICLEVRPNGRPINTAREPPLTQALI
PGDVFAKNSLWKGAYEYQGKKQPAMLRVTGFQVANSKVNATMIDHSGVELHLAGTYKKEDFHLLLQVY
QITGPVEIFMNKFKDDHWALDGHVSSESSGATFIYQGSVKGQGFGQFGFQRLDLRLLESDPESIGRHF
ASNSSSVAAAILVPFIALIIAGFVLYLYKHRRRPKVPFNGYAGHENTNVRATFENPMYDRNIQPTDIM
ASEAEFTVSTVCTAV

| | | |
|---|---|---|
| NOV4f, CG50377-06 | SEQ ID NO: 41 | 12901 bp |
| DNA Sequence | ORF Start: ATG at 889 | ORF Stop: TAG at 10279 |

GGCCAGAACTGCACGTTCCAACTGCACGGTCCCAATGGGACAGTTGAGAGCCCAGGGTTCCCATATGG
CTACCCCAATTACGCCAACTGCACGTGGACCATCACCGCGGAAGAGCAGCACAGAATCCAGCTTGTGT
TCCAGTCCTTTGCCCTGGAAGAGGACTTTGATGTCCTGTCGGTGTTTGATGGTCCACCCCAGCCAGAG
AATCTGCGTACGAGGCTCACAGGCTTTCAGCTGCCAGCCACCATTGTTAGTGCAGCCACCACCCTCTC
TCTGCGCCTCATCAGCGACTATGCAGTCAGTGCCCAAGGCTTCCACGCCACCTATGAAGTTCTCCCCA
GCCACACATGTGGGAACCCAGGGAGGCTGCCCAATGGCATCCAGCAGGGTTCAACCTTCAACCTCGGT
GACAAGGTCCGCTACAGCTGCAACCTTGGCTTCTTCCTGGAGGGCCACGCCGTGCTCACCTGCCACGC
TGGCTCTGAGAACAGCGCCACGTGGGACTTCCCCCTGCCTTCCTGCAGAGCTGATGATGCCTGTGGTG
GGACCCTGCGGGGCCAGAGTGGCATCATCTCCAGCCCCCACTTCCCCTCGGAGTACCATAACAATGCC
GACTGCACATGGACCATCCTGGCTGAGCTGGGGGACACCATCGCCCTGGTGTTTATTGACTTCCAGCT
GGAGGATGGTTACGACTTTCTGGAAGTCACTGGGACAGAAGGCTCCTCCCTCTGGTTCACCGGAGCCA
GCCTCCCAGCCCCCGTTATCAGCAGCAAGAACTGGCTGCGACTGCACTTCACATCGGATGGCAACCAC
CGGCAGCGCGGATTCAGTGCCCAATACCAAGTCAAGAAGCAAATTGAGTTGAAGTCTCGAGGTGTGAA
GCTGATGCCCAGCAAAGACAACAGCCAGAAGACGTCTGTGGTAACTCAGGTTGGTGTGTCCCAAGGAC
ATAATATGTGTCCAGACCCTGGCATACCCGAAAGGGGCAAAAGACTAGGCTCGGATTTCAGGTTAGGA
TCCAGCGTCCAGTTCACCTGCAACGAGGGCTATGACCTGCAAGGGTCCAAGCGGATCACCTGTATGAA
AGTGAGCGACATGTTTGCGGCCTGGAGCGACCACAGGCCAGTCTGCCGAGCCCGCATGTGTGATGCCC
ACCTTCGAGGCCCCTCGGGCATCATCACCTCCCCCAATTTCCCCATTCAGTATGACAACAATGCACAC
TGTGTGTGGATCATCACAGCACTCAACCCCTCCAAGGTAATCAAGCTCGCCTTTGAGGAGTTTGATTT
GGAGAGGGGCTATGACACCCTGACGGTCGGTGATGGTGGTCAGGATGGGGACCAGAAGACAGTTCTCT
ACATGCTGACAGGTACATCGGTCCCGGATCTCATTGTCAGCACCAATCATCAAATGTGGCTCCTCTTC
CAGACTGATGGCAGTGGCAGTTCCCTGGGATTCAAGGCTTCTTATGAAGAGATCGAGCAGGGCAGTTG
CGGTGACCCTGGCATACCTGCATATGGCCGGAGGGAAGGCTCCCGGTTTCGCCACGGTGACACACTCA
AGTTTGAGTGCCAGCCCGCCTTTGAGCTGGTGGGACAGAAGGCAATCACATGCCAAAAGAATAACCAA
TGGTCGGCTAAGAAGCCAGGCTGCGTGTGTTCCTGCTTCTTCAACTTCACCAGCCCGTCTGGGGTTGT
CCTGTCTCCCAACTACCCAGAGGACTATGGCAACCACCTCCACTGTGTCTGGCTCATCCTGGCCAGGC
CTGAGAGCCGCATCCACCTGGCCTTCAACGACATTGACGTGGAGCCTCAGTTTGATTTCCTGGTCATC
AAGGATGGGGCCACCGCCGAGGCGCCCGTCCTGGGCACCTTCTCAGGAAACCAGCTTCCCTCCTCCAT
CACAAGCAGTGGCCACGTGGCCCGTCTCGAGTTCCAGACTGACCACTCCACAGGGAAGAGGGGCTTCA
ACATCACTTTTACCAGTGAGTCCTCCAACGAGTGCCCGGATCCTGGCGTTCCAGTAAATGGCAAACGG
TTTGGGGACAGCCTCCAGCTGGGCAGCTCCATCTCCTTCCTCTGTGATGAAGGCTTCCTTGGGACTCA
GGGCTCAGAGACCATCACCTGCGTCCTGAAGGAGGGCAGCGTGGTCTGGAACAGCGCTGTGCTGCGGT
GTGAAGCTCCCTGTGGTGGTCACCTGACTTCGCCCAGCGGCACCATCCTCTCTCCGGGCTGGCCTGGC
TTCTACAAGGATGCCTTGAGCTGTGCCTGGGTGATTGAGGCCCAGCCAGGCTACCCCATCAAAATCAC
CTTCGACAGGTTCAAAACCGAGGTCAACTATGACACCCTGGAAGTACGCGATGGGCGGACTTACTCAG
CGCCCTTGATCGGGGTTTACCACGGGACCCAGGTTCCCCAGTTCCTCATCAGCACCAGCAACTACCTC
TACCTCCTCTTCTCTACCGACAAGAGTCACTCGGACATCGGCTTCCAGCTCCGCTATGAGGCTATAAC
ACTGCAGTCAGACCACTGTCTGGATCCAGGAATCCCAGTAAATGGACAGCGTCATGGGAATGACTTCT
ACGTGGGCGCGCTGGTGACCTTCAGCTGTGACTCGGGCTACACATTAAGTGACGGGGAGCCTCTGGAG
TGTGAGCCCAACTTCCAGTGGAGCCGGGCCCTGCCCAGTTGTGAAGCTCTCTGTGGTGGCTTCATTCA
AGGCTCCAGTGGGACCATCTTGTCGCCAGGTTTCCCTGACTTCTACCCCAACAACTTGAACTGCACCT
GGATTATCGAAACATCTCATGGCAAGGGTGTGTTCTTCACTTTCCACACCTTCCACCTGGAAAGTGGC
CATGACTACCTCCTCATCACTGAGAACGGCAGCTTCACCCAGCCCCTGAGGCAGCTAACTGGATCTCG
GCTGCCAGCTCCCATCAGCGCTGGGCTCTATGGCAACTTCACTGCCCAGGTCCAATTCATCTCTGATT
TCTCCATGTCATATGAAGGATTCAACATCACCTTCTCAGAGTACGACTTGGAGCCCTGTGAGGAGCCC
GAGGTCCCAGCCTACAGCATCCGGAAGGGCTTGCAGTTTGGCGTGGGCGACACCTTGACCTTCTCCTG
CTTCCCCGGGTACCGTCTGGAGGGCACCGCCCGCATCACGTGCCTGGGGGGCAGACGGCGCCTGTGGA
GCTCGCCTCTGCCAAGGTGTGTTGCTGAGTGTGGGAATTCAGTCACAGGCACTCAGGGTACTTTGCTG
TCCCCCAACTTTCCTGTGAACTACAATAACAATCATGAATGCATCTACTCCATCCAGACCCAGCCAGG
GAAGGGAATTCAGCTGAAAGCCAGGGCATTCGAACTCTCCGAAGGAGATGTCCTCAAGGTTTATGATG
GCAACAACAACTCCGCCCGTTTGCTGGGAGTTTTTAGCCATTCTGAGATGATGGGGGTGACTTTGAAC
AGCACATCCAGCAGTCTGTGGCTTGATTTCATCACTGATGCTGAAAACACCAGCAAGGGCTTTGAACT
GCACTTTTCCAGTTTTGAACTCATCAAATGTGAGGACCCAGGAACCCCCAAGTTTGGCTACAAGGTTC
ATGATGAAGGTCATTTTGCAGGGAGCTCCGTGTCCTTCAGCTGTGACCCTGGATACAGCCTGCGGGGT
AGTGAGGAGCTGCTGTGTCTGAGTGGAGAGCGCCGGACCTGGGACCGGCCTCTGCCCACCTGTGTCGC
CGAGTGTGGAGGGACAGTGAGAGGAGAGGTGTCGGGGCAGGTGCTGTCACCCGGGTATCCAGCTCCCT
ATGAACACAATCTCAACTGCATCTGGACCATCGAAGCAGAGGCCGGCTGCACCATTGGGCTACACTTC
CTGGTGTTTGACACAGAGGAGGTTCACGACGTGCTGCGCATCTGGGATGGGCCTGTGGAGAGCGGGGT
TCTGCTGAAGGAGCTGAGTGGCCCGGCCCTGCCCAAGGACCTGCATAGCACCTTCAACTCGGTCGTCC
TGCAGTTCAGCACTGACTTCTTCACCAGCAAGCAGGGCTTTGCCATTCAATTTTCAGGTTCCACAGCA
ACGTCCTGCAATGACCCTGGGATCCCGCAGAATGGGAGTCGGAGTGGTGACAGTTGGGAAGCCGGCGA
CTCCACAGTGTTCCAGTGTGACCCTGGCTACGCGCTGCAGGGAAGTGCAGAGATCAGCTGTGTGAAGA
TCGAGAACAGGTTCTTCTGGCAGCCCAGCCCGCCAACATGCATCGCTCCCTGCGGGGGAGACCTGACA
GGACCATCTGGAGTCATCCTCTCACCAAATTACCCAGAACCCTACCCGCCAGGCAAGGAGTGTGACTG
GAAAGTGACCGTCTCACCAGACTACGTCATCGCCCTGGTATTGTTCCCCAGCTTTAACCTGGAGCCTG
GCTATGACTTCCTCCATATCTACGACGGACGGGACTCTCTCAGCCCTCTCATAGGAAGCTTCTATGGC
TCCCAGCTCCCAGGCCGCATTGAAAGCAGCAGCAACAGCCTCTTCCTCGCCTTCCGCAGCGATGCATC
TGTGAGCAATGCTGGCTTCGTCATTGACTTCCCAGAAAACCCGCGGGAGTCATGTTTTGATCCTGGTT
CCATCAAGAACGGCACACGGGTGGGGTCCGACCTGAAGCTGGGCTCCTCCGTCACCTACTACTGCCAC
GGGGGCTACGAAGTTGAGGGCACCTCGACCCTGAGCTGCATCCTGGGGCCTGATGGGAAGCCCGTGTG
GAACAATCCCCGGCCAGTCTGCACAGCCCCCTGTGGGGGACAGTATGTGGGTTCGGACGGAGTGGTCT
TGTCCCCCAACTACCCCCAGAACTACACCAGTGGACAGATCTGCTTGTATTTTGTTACTGTGCCCAAG
GACTATGGTGTGGTGTTTGGCCAGTTCGCCTTCTTTCACACGGCCCTCAACGACGTGGTGGAGGTTCA
CGACGGCCACAGCCAGCACTCGCGGCTCCTCAGCTCCCTCTCGGGCTCCCATACAGGTGGAGAATCAC
TGCCCTTGGCCACCTCCAATCAAGTTCTCATTAAGTTCAGCGCCAAAGGCCTCGCACCAGCCAGAGGC
TTCCACTTTGTCTACCAAGCGGTTCCTCGAACCAGCGCCACGCAGTGCAGCTCTGTGCCGGAACCCCG
CTATGGCAAGAGGCTGGGCAGTGACTTCTCGGTGGGGGCCATCGTCCGCTTCGAATGCAACTCCGGCT
ATGCCCTGCAGGGGTCGCCAGAGATCGAGTGCCTCCCTGTGCCTGGGGCCTTGGCCCAATGGAATGTC
TCAGCGCCCACGTGTGTGGTGCCGTGTGGAGGCAACCTCACAGAGCGCAGGGGCACCATCCTGTCCCC
TGGCTTCCCAGAGCCGTACCTCAACAGCCTCAACTGTGTGTGGAAGATCGTGGTCCCCGAAGGCGCTG
GCATCCAGATCCAAGTTGTCAGTTTTGTGACAGAGCAGAACTGGGACTCGCTGGAAGTATTTGATGGT
GCAGATAACACTGTAACCATGCTGGGGAGTTTCTCAGGAACAACCGTGCCTGCCCTTCTGAACAGCAC
CTCCAACCAGCTCTACCTTCATTTCTACTCAGATATCAGCGTATCTGCAGCTGGCTTCCACTTGGAGT
ACAAAACGGTGGGCCTGAGCAGTTGTCCGGAACCTGCTGTGCCCAGTAACGGGGTGAAGACTGGCGAG
CGCTACTTGGTGAATGATGTGGTGTCTTTCCAGTGTGAGCCGGGATATGCCCTCCAGGGCCACGCCCA
CATCTCCTGCATGCCCGGAACAGTGCGGCGATGGAACTACCCTCCTCCACTCTGTATTGCACAGTGTG
GGGGAACAGTGGAGGAGATGGAGGGGGTGATCCTGAGCCCCGGCTTCCCAGGCAACTACCCCAGTAAC
ATGGACTGCTCCTGGAAAATAGCACTGCCCGTGGGCTTTGGAGCTCACATCCAGTTCCTGAACTTCTC
CACCGAGCCCAACCACGACTACATAGAAATCCGGAATGGCCCCTATGAGACCAGCCGCATGATGGGAA
GATTCAGTGGAAGCGAGCTTCCAAGCTCCCTCCTCTCCACGTCCCACGAGACCACCGTGTATTTCCAC
AGCGACCACTCCCAGAATCGGCCAGGATTCAAGCTGGAGTATCAGGCCTATGAACTTCAAGAGTGCCC
AGACCCAGAGCCCTTTGCCAATGGCATTGTGAGGGGAGCTGGCTACAACGTGGGACAATCAGTGACCT
TCGAGTGCCTCCCGGGGTATCAATTGACTGGCCACCCTGTCCTCACGTGTCAACATGGCACCAACCGG
AACTGGGACCACCCCCTGCCCAAGTGTGAAGTCCCTTGTGGCGGGAACATCACTTCTTCCAACGGCAC
TGTGTACTCCCCGGGGTTCCCTAGCCCGTACTCCAGCTCCCAGGACTGTGTCTGGCTGATCACCGTGC
CCATTGGCCATGGCGTCCGCCTCAACCTCAGCCTGCTGCAGACAGAGCCCTCTGGAGATTTCATCACC
ATCTGGGATGGGCCACAGCAAACAGCACCACGGCTCGGCGTCTTCACCCGGAGCATGGCCAAGAAAAC
AGTGCAGAGTTCATCCAACCAGGTCCTGCTCAAGTTCCACCGTGATGCAGCCACAGGGGGGATCTTCG
CCATAGCTTTCTCCGCTTATCCACTCACCAAATGCCCTCCTCCCACCATCCTCCCCAACGCCGAAGTC
GTCACAGAGAATGAAGAATTCAATATAGGTGACATCGTACGCTACAGATGCCTCCCTGGCTTTACCTT
AGTGGGGAATGAAATTCTGACCTGCAAACTTGGAACCTACCTGCAGTTTGAAGGACCACCCCCGATAT
GTGAAGTGCACTGTCCAACAAATGAGCTTCTGACAGACTCCACAGGCGTGATCCTGAGCCAGAGCTAC
CCTGGAAGCTATCCCCAGTTCCAGACCTGCTCTTGGCTGGTGAGAGTGGAGCCCGACTATAACATCTC
CCTCACAGTGGAGTACTTCCTCAGCGAGAAGCAATATGATGAGTTTGAGATTTTTGATGGTCCATCAG
GACAGAGTCCTCTGCTGAAAGCCCTCAGTGGGAATTACTCAGCTCCCCTGATTGTCACCAGCTCAAGC
AACTCTGTGTACCTGCGTTGGTCATCTGATCACGCCTACAATCGGAAGGGCTTCAAGATCCGCTATTC
AGCCCCTTACTGCAGCCTGCCCAGGGCTCCACTCCATGGCTTCATCCTAGGCCAGACCAGCACCCAGC
CCGGGGGCTCCATCCACTTTGGCTGCAACGCCGGCTACCGCCTGGTGGGACACAGCATGGCCATCTGT
ACCCGGCACCCCCAGGGCTACCACCTGTGGAGCGAAGCCATCCCTCTCTGTCAAGCTCTTTCCTGTGG
GCTTCCTGAGGCCCCCAAGAATGGAATGGTGTTTGGCAAGGAGTACACAGTGGGAACCAAGGCCATGT
ACAGCTGCAGTGAAGGCTACCACCTCCAGGCAGGCGCTGAGGCCACTGCAGAGTGTCTGGACACAGGC
CTATGGAGCAACCGCAATGTCCCACCACAGTGTGTCCCTGTGACTTGTCCTGATGTCAGTAGCATCAG
CGTGGAGCATGGCCGATGGAGGCTTATCTTTGAGACACAGTATCAGTTCCAGGCCCAGCTGATGCTCA
TCTGTGACCCTGGCTACTACTATACTGGCCAAAGGGTCATCCGCTGTCAGGCCAATGGCAAATGGAGC
CTCGGGGACTCTACGCCCACCTGCCGAATCATCTCCTGTGGAGAGCTCCCGATTCCCCCCAATGGCCA
CCGCATCGGAACACTGTCTGTCTACGGGGCAACAGCCATCTTCTCCTGCAATTCCGGATACACACTGG
TGGGCTCCAGGGTGCGTGAGTGCATGGCCAATGGGCTCTGGAGTGGCTCTGAAGTCCGCTGCCTTGCT
GGACACTGTGGGACTCCTGAGCCCATTGTCAACGGACACATCAATGGGGAGAACTACAGCTACCGGGG
CAGTGTGGTGTACCAATGCAATGCTGGCTTCCGCCTGATCGGCATGTCTGTGCGCATCTGCCAGCAGG
ATCATCACTGGTCGGGCAAGACCCCTTTCTGTGTGCTGGTGTCCTGTGGCCATCCGGGCTCCCCGCCT
CACTCCCAGATGTCTGGAGACAGTTATACTGTGGGAGCAGTGGTGCGGTACAGCTGCATCGGCAAGCG
TACTCTGGTGGGAAACAGCACCCGCATGTGTGGGCTGGATGGACACTGGACTGGCTCCCTCCCTCACT
GCTCAGGAACCAGCGTGGGAGTTTGCGGTGACCCTGGGATCCCGGCTCATGGCATCCGTTTGGGGGAC
AGCTTTGATCCAGGCACTGTGATGCGCTTCAGCTGTGAAGCTGGCCACGTGCTCCGGGGATCGTCAGA
GCGCACCTGTCAAGCCAATGGCTCGTGGAGCGGCTCGCAGCCTGAGTGTGGAGTGATCTCTTGTGGGA
ACCCTGGGACTCCAAGTAATGCCCGAGTTGTGTTCAGTGATGGCCTGGTTTTCTCCAGCTCTATCGTC
TATGAGTGCCGGGAAGGATACTACGCCACAGGCCTGCTCAGCCGTCACTGCTCGGTCAATGGTACCTG
GACAGGCAGTGACCCTGAGTGCCTCGTCATAAACTGTGGTGACCCTGGGATTCCAGCCAATGGCCTTC
GGCTGGGCAATGACTTCAGGTACAACAAAACTGTGACATATCAGTGTGTCCCTGGCTATATGATGGAG
TCACATAGAGTATCTGTGCTGAGCTGCACCAAGGACCGGACATGGAATGGAACCAAGCCCGTCTGCAA
AGCTCTCATGTGCAAGCCACCTCCGCTCATCCCCAATGGGAAGGTGGTGGGGTCTGACTTCATGTGGG
GCTCAAGTGTGACTTATGCCTGCCTGGAGGGGTACCAGCTCTCCCTGCCCGCGGTGTTCACCTGTGAG
GGAAATGGGTCCTGGACCGGAGAGCTGCCTCAGTGTTTCCCTGTGTTCTGCGGGGATCCTGGTGTCCC
GTCCCGTGGGAGGAGAGAGGACCGAGGCTTCTCCTACAGGTCATCTGTCTCCTTCTCCTGCCATCCCC
CTCTGGTGCTGGTGGGCTCTCCACGCAGGTTTTGCCAGTCAGATGGGACATGGAGTGGCACCCAGCCC
AGCTGCATAGATCCGACCCTGACCACGTGTGCGGACCCTGGTGTGCCACAGTTTGGGATACAGAACAA
TTCTCAGGGCTACCAGGTTGGAAGCACAGTCCTCTTCCGTTGTCAAAAAGGCTACCTGCTTCAGGGCT
CCACCACCAGGACCTGCCTCCCAAACCTGACCTGGAGTGGAACCCCACCTGACTGTGTCCCCCACCAC
TGCAGGCAGCCAGAGACGCCAACGCATGCCAACGTCGGGGCCCTGGATTTGCCCTCCATGGGCTACAC
GCTCATCTACTCCTGCCAGGAGGGCTTCTCCCTCAAGGGTGGCTCCGAGCACCGCACCTGCAAGGCGG
ATGGCAGCTGGACAGGCAAGCCGCCCATCTGCCTGGAGGTCCGGCCCAATGGGAGACCCATCAACACT
GCCCGGGAGCCACCGCTCACCCAAGCCTTGATTCCTGGGGATGTTTTTGCCAAGAATTCCCTGTGGAA
AGGGGCCTATGAATACCAGGGGAAGAAGCAGCCAGCCATGCTCAGAGTGACTGGCTTCCAAGTTGCCA
ACAGCAAGGTCAATGCCACCATGATCGACCACAGTGGCGTGGAGCTGCACTTGGCTGGAACTTACAAG
AAAGAAGATTTTCATCTCCTACTCCAGGTGTACCAGATTACAGGGCCTGTGGAGATCTTTATGAATAA
GTTCAAAGATGATCACTGGGCTTTAGATGGCCATGTCTCGTCAGAGTCCTCCGGAGCCACCTTCATCT
ACCAAGGCTCTGTCAAGGGCCAAGGCTTTGGGCAGTTCGGCTTTCAAAGACTGGACCTCAGGCTGCTG
GAGTCAGACCCCGAGTCCATTGGCCGCCACTTTGCTTCCAACAGCAGCTCAGTGGCAGCCGCGATCCT
GGTGCCTTTCATCGCCCTCATTATTGCGGGCTTCGTGCTCTATCTCTACAAGCACAGGAGAAGACCCA
AAGTTCCTTTCAATGGCTATGCTGGCCACGAGAACACCAATGTTCGGGCCACATTTGAGAACCCAATG
TACGACCGCAACATCCAGCCCACAGACATCATGGCCAGCGAGGCGGAGTTCACAGTCAGCACAGTGTG
CACAGCAGTATAGCCACCCGGCCTGGCCGCCACCACCGCCACCGAGAGCCCCGCCTCCAGCAGCCGTT
CCCCAGGGGGCAGGAAGTAGGCTCCCCTGAGCACAGTGCCCAGTGCCCATCTTCGTTCGTCTCCTTGC
CTGTGTCTGCTACTGCTGGGACTCAGGCCCTCGCCCTGCCGCCATCTATGCCAGTTACCCTGGAGAGG
ACTCCTGCCGTGTCCTGAAAAGGCAGAAAAGGAAGAGGGGCTGCAGACAGAGGAAGAAAGTTTATGGA
TTTAAAATAGCAAAGCAACTTGTGGATCTGTGTGTGTGTGTGTGTGTGTGTGTATGGGCCTGATCACA
CACAAACACACACACCTCTCCTTCATTCACGTCCTATGGAAGAGACCATAAAGGCTTCAAAACTAGAA
GTGCGCATGAAGGATGCTTCATGCCCTGTTCCCCACACAGAGAAGAGATGAAGAGGGGGAGGTACGGT
CAGCTTTCAAAACAATATGCCCCCCGCCCATCCCATGTGTGAGGACATGGTGACTCGGTGACTGTAAC
AGTAGGACACATCCCATCATGCGGTCAGAGTGCAGAAACCCGGTCAGCGTGCAGAAACCAGCTGTCTG
CTGATGAACGGCCCATCTGCACAGCGGCAGCAAGGATTACCCCAAGGGCAGGAAAAAAGTCTTTGGAP
ATAGATTGAAAATCAGTTTCTTTATTTTTTTCAAACACTGTTTTATTGAGTTCTAAACCCAGTGGATT
AAAAAGTCATTTGGGTCCTGGGAAGCACTGGGAAAGAAGGGAGAGCCGGGGAGGATAGGGCAGGAGTC
TGGCCAGGAGCCCAAGGTCAAGTCTGGCTGTCAACTGACAAAGAGAAGAGAAGACAAGTCTGTGGCCC
AGACAGGGTGCCAAGTCTGGGGGCAGCAGCAGGGTCCTTGAGAGAACAGGGGAGCAAGCCTGCCCCAG
TACCCAAAAAACAATGGCTATCATGTACTGGGGTTATTCTCTGTGCCTGACACTTTACCTATAGTATT
TCTAGTCTGCACAATAACACCAAAATGTAGGATTTGTTATTCCCCTTTGACAGATGAGAAAACAGAAG
CCTGCAAAGGTAAAGACAGAGTTAGGAAGTGTGGAGTCAGAATGCAAACCCAGATCAGTCTGCCCCAA
AGTCCACTACACCAGCTGCCAGAAACCCTGTTCTAGAAGGGGCAGCTAAGAAGGGAGGAGCCGATGAA
ACTCTACAGTAGCACTGTGGCCAGGTCTGGAAAGCCAGGCAAGACACCTGTGTGTCCTATCTGGGTTC
TGCTGTCTCCGCGTAGGCAGCTTCGGGGCAAGGATTCAACTCCCCAGGCCTCCCACGGTTGGTGCTGG
CTCCTTGGCTCCCAGCAGCCGCTGACCATAGCCAGCAAAAGACCTGGCAGGTCCTGAATCAAAACAAC
AGAGTGGCTGAAAGTCACACCTCTCCAGCTCCTTGCGAGTGGGTTTGATTGATTTTGACAGCTATATC
ATGCTTGGGTCGTTCATGAATTCCATGAGAATTGTTGGGAAGTTCATGAGTTTAGGAATCAAAGTATA
GCATTTAGGGCCACTTTCTCTGTCCTCGGGAAGGCCCCCACAAGCTTAAGCCCCTGTATACGTGTGAC
ATGCTGGCCTCAGAGATTCATGTCACATATTGAAATAGCAAATAGAAATGTGCAGGAGCGGAGGGACA
GCGACACCTGGTTCTTAAGCAAATTCTAGATTGGAACAATTAGAAGATCTGACTGATGTTAAGAGTGG
CCAGAGGAAAGTGAGAGGCGTCCATTTCCAGCAGACACAATGGAGGCTGACAGAGGCAGGAGCCTACC
CCTGGCCACACCACCTCTGGGACACGGCACCCCCCTGGGCCTGGAGGGAGAGGGCCAATCCAGACACA
CTCACACTCTCATCTCTTTCCTTGGCTGGAAACAAGTGCAGGAAGCTCCCCTCCCAATTTCTTATGCT
CCCTTCCCCCTTACACTCCTCTCCTTCGACTGCTCCCTTCCCCCTTCTTATGCCCCTCTCAGACCACC
CAGCGGATGCCTATTCCCGCCCCCCACCCCTCCCCTGCGTCTCCTTGTAAAGTTCCATGAGATGCTCG
AGGAGCACCTGCTGGTGGGGCACCCAGCACTCTGCTGGGCATCACCCTCCGCACCCCTGACATTTCTC
TCCACCTCCAGAGGCTTGGAAGGGAACAGGGCAGATGAGGCCCTTCACCCACCATCTACAACATATAC
CCATCACCATCCCATGAGGAAAAAAAAAAAAGTAAAACAAAGACAAAAAAAATCTTTTGTACAGAGAT
ATATTTTTATTATACAAAGTTTGTACAGTACCAAAAAAGAAAAAAAAAAGGTGTAAATTTTTTTTTCA
TTATTGTAGGTAAACGGTAGTGGAAGCCTTTTTTGTAAATGTAATAAAATGTATAAATATGGTTTTCA
GAAAACATCAAGTGCTGTAAATATGTAATCTACACACCTTCTTGGCTTGTCTGCAGTATATACATTTA
ATTTATCTGTCTCTGTATATGAGGCTTCCCCCTGGGGTCCTGCATTATGGTACTTTCCTGAATTTGAA
AGCAATTTTAAATTTTTTGAATTCAAATAAAATATAAATTTTTGCATTG

| | | | |
|---|---|---|---|
| NOV4f, CG50377-06 | SEQ ID NO: 42 | 3130 aa | MW at 340344.5kD |
| Protein Sequence | | | |

MPSKDNSQKTSVVTQVGVSQGHNMCPDPGIPERGKRLGSDFRLGSSVQFTCNEGYDLQGSKRITCMKV
SDMFAAWSDHRPVCPARMCDAHLRGPSGIITSPNFPIQYDNNAHCVWIITALNPSKVIKLAFEEFDLE
RGYDTLTVGDGGQDGDQKTVLYMLTGTSVPDLIVSTNHQMWLLFQTDGSGSSLGFKASYEEIEQGSCG
DPGIPAYGRREGSRFRHGDTLKFECQPAFELVGQKAITCQKNNQWSAKKPGCVCSCFFNFTSPSGVVL
SPNYPEDYGNHLHCVWLILARPESRIHLAFNDIDVEPQFDFLVIKDGATAEAPVLGTFSGNQLPSSIT
SSGHVARLEFQTDHSTGKRGFNITFTSESSNECPDPGVPVNGKRFGDSLQLGSSISFLCDEGFLGTQG
SETITCVLKEGSVVWNSAVLRCEAPCGGHLTSPSGTILSPGWPGFYKDALSCAWVIEAQPGYPIKITF
DRFKTEVNYDTLEVPDGRTYSAPLIGVYHGTQVPQFLISTSNYLYLLFSTDKSHSDIGFQLRYEAITL
QSDHCLDPGIPVNGQRHGNDFYVGALVTFSCDSGYTLSDGEPLECEPNFQWSRALPSCEALCGGFIQG
SSGTILSPGFPDFYPNNLNCTWIIETSHGKGVFFTFHTFHLESGHDYLLITENGSFTQPLRQLTGSRL
PAPISAGLYGNFTAQVRFISDFSMSYEGFNITFSEYDLEPCEEPEVPAYSIRKGLQFGVGDTLTFSCF
PGYRLEGTARITCLGGRRRLWSSPLPRCVAECGNSVTGTQGTLLSPNFPVNYNNNHECIYSIQTQPGK
GIQLKAEAFELSEGDVLKVYDGNNNSARLLGVFSHSEMMGVTLNSTSSSLWLDFITDAENTSKGFELH
FSSFELIKCEDPGTPKFGYKVHDEGHFAGSSVSFSCDPGYSLRGSEELLCLSGERRTWDRPLPTCVAE
CGGTVRGEVSGQVLSPGYPAPYEHNLNCIWTIEAEAGCTIGLHFLVFDTEEVHDVLRIWDGPVESGVL
LKELSGPALPKDLHSTFNSVVLQFSTDFFTSKQGFAIQFSGSTATSCNDPGIPQNGSRSGDSWEAGDS
TVFQCDPGYALQGSAEISCVKIENRFFWQPSPPTCIAPCGGDLTGPSGVILSPNYPEPYPPGKECDWK
VTVSPDYVIALVLFPSFNLEPGYDFLHIYDGRDSLSPLIGSFYGSQLPGRIESSSNSLFLAFRSDASV
SNAGFVIDFPENPRESCFDPGSIKNGTRVGSDLKLGSSVTYYCHGGYEVEGTSTLSCILGPDGKPVWN
NPRPVCTAPCGGQYVGSDGVVLSPNYPQNYTSGQICLYFVTVPKDYGVVFGQFAFFHTALNDVVEVHD
GHSQHSRLLSSLSGSHTGGESLPLATSNQVLIKFSAKGLAPARGFHFVYQAVPRTSATQCSSVPEPRY
GKRLGSDFSVGAIVRFECNSGYALQGSPEIECLPVPGALAQWNVSAPTCVVPCGGNLTERRGTILSPG
FPEPYLNSLNCVWKIVVPEGAGIQIQVVSFVTEQNWDSLEVFDGADNTVTMLGSFSGTTVPALLNSTS
NQLYLHFYSDISVSAAGFHLEYKTVGLSSCPEPAVPSNGVKTGERYLVNDVVSFQCEPGYALQGHAHI
SCMPGTVRRWNYPPPLCIAQCGGTVEEMEGVILSPGFPGNYPSNMDCSWKIALPVGFGAHIQFLNFST
EPNHDYIEIRNGPYETSRMMGRFSGSELPSSLLSTSHETTVYFHSDHSQNRPGFKLEYQAYELQECPD
PEPFANGIVRGAGYNVGQSVTFECLPGYQLTGHPVLTCQHGTNRNWDHPLPKCEVPCGGNITSSNGTV
YSPGFPSPYSSSQDCVWLITVPIGHGVRLNLSLLQTEPSGDFITIWDGPQQTAPRLGVFTRSMAKKTV
QSSSNQVLLKFHRDAATGGIFAIAFSAYPLTKCPPPTILPNAEVVTENEEFNIGDIVRYRCLPGFTLV
GNEILTCKLGTYLQFEGPPPICEVHCPTNELLTDSTGVILSQSYPGSYPQFQTCSWLVRVEPDYNISL
TVEYFLSEKQYDEFEIFDGPSGQSPLLKALSGNYSAPLIVTSSSNSVYLRWSSDHAYNRKGFKIRYSA
PYCSLPRAPLHGFILGQTSTQPGGSIHFGCNAGYRLVGHSMAICTRHPQGYHLWSEAIPLCQALSCGL
PEAPKNGMVFGKEYTVGTKAMYSCSEGYHLQAGAEATAECLDTGLWSNRNVPPQCVPVTCPDVSSISV
EHGRWRLIFETQYQFQAQLMLICDPGYYYTGQRVIRCQANGKWSLGDSTPTCRIISCGELPIPPNGHR
IGTLSVYGATAIFSCNSGYTLVGSRVRECMANGLWSGSEVRCLAGHCGTPEPIVNGHINGENYSYRGS
VVYQCNAGFRLIGMSVRICQQDHHWSGKTPFCVLVSCGHPGSPPHSQMSGDSYTVGAVVRYSCIGKRT
LVGNSTRMCGLDGHWTGSLPHCSGTSVGVCGDPGIPAHGIRLGDSFDPGTVMRFSCEAGHVLRGSSER
TCQANGSWSGSQPECGVISCGNPGTPSNARVVFSDGLVFSSSIVYECREGYYATGLLSRHCSVNGTWT
GSDPECLVINCGDPGIPANGLRLGNDFRYNKTVTYQCVPGYMMESHRVSVLSCTKDRTWNGTKPVCKA
LMCKPPPLIPNGKVVGSDFMWGSSVTYACLEGYQLSLPAVFTCEGNGSWTGELPQCFPVFCGDPGVPS
RGRREDRGFSYRSSVSFSCHPPLVLVGSPRRFCQSDGTWSGTQPSCIDPTLTTCADPGVPQFGIQNNS
QGYQVGSTVLFRCQKGYLLQGSTTRTCLPNLTWSGTPPDCVPHHCRQPETPTHAINGALDLPSMGYTL
IYSCQEGFSLKGGSEHRTCKADGSWTGKPPICLEVRPNGRPINTAREPPLTQALIPGDVFAKNSLWKG
AYEYQGKKQPAMLRVTGFQVANSKVNATMIDHSGVELHLAGTYKKEDFHLLLQVYQITGPVEIFMNKF
KDDHWALDGHVSSESSGATFIYQGSVKGQGFGQFGFQRLDLRLLESDPESIGRHFASNSSSVAAAILV
PFIALIIAGFVLYLYKHRRRPKVPFNGYAGHENTNVRATFENPMYDRNIQPTDIMASEAEFTVSTVCT
AV

| | | |
|---|---|---|
| NOV4g, 273095147 | SEQ ID NO: 43 | 2329 bp |
| DNA Sequence | ORF Start: at 1 | ORF Stop: at 2329 |

AAGCTTGAGTGCCCAGACCCAGAGCCCTTTGCCAATGGCATTGTGAGGGGAGCTGGCTACAACGTGGG
ACAATCAGTGACCTTCGAGTGCCTCCCGGGGTATCAATTGACTQGCCACCCTGTCCTCACGTGTCAAC
ATGGCACCAACCGGAACTGGGACCACTCCCTGCCCAAGTGTGAAGTCCCTTGTGGCGGGAACATCACT
TCTTCCAACGGCACTGTGTACTCCCCGGGGTTCCCTAGCCCGTACTCCAGCTCCCAGGACTGTGTCTG
GCTGATCACCGTGCCCATTGGCCATGGCGTCCGCCTCAACCTCAGCCTGCTGCAGACAGAGCCCTCTG
GAGATTTCATCACCATCTGGGATGGGCCACAGCAAACAGCACCACGGCTCGGCGTCTTCACCCGGAGC
ATGGCCAAGAAAACAGTGCAGAGTTCATCCAACCAGGTCCTGCTCAAGTTCCACCGTGATGCAGCCAC
AGGGGGGATCTTCGCCATAGCTTTCTCCGCTTATCCACTCACCAAATGCCCTCCTCCCACCATCCTCC
CCAACGCCGAAGTCGTCACAGAGAATGAAGAATTCAATATAGGTGACATCGTACGCTACAGATGCCTC
CCTGGCTTTACCTTAGTGGGGAATGAAATTCTTACCTGCAAACTTGGAACCTACCTGCAGTTTGAAGG
ACCACCCCCGATATGTGAAGTGCACTGTCCAACAAATGAGCTTCTGACAGACTCCACAGGCGTGATCC
TGAGCCAGAGCTACCCTGGAAGCTATCCCCAGTTCCAGACCTGCTCTTGGCTGGTGAGAGTGGAGCCC
GACTATAACATCTCCCTCACAGTGGAGTACTTCCTCAGCGAGAAGCAATATGATGAGTTTGAGATTTT
TGATGGTCCATCAGGACAGAGTCCTCTGCTGAAAGCCCTCAGTGGGAATTACTCAGCTCCCCTGATTG
TCACCAGCTCAAGCAACTCTGTGTACCTGCGTTGGTCATCTGATCACGCCTACAATCGGAAGGGCTTC
AAGATCCGCTATTCAGCCCCTTACTGCAGCCTGCCCAGGGCTCCACTCCATGGCTTCATCCTAGGCCA
GACCAGCACCCAGCCCGGGGGCTCCATCCACTTTGGCTGCAACGCCGGCTACCGCCTGGTGGGACACA
GCATGGCCATCTGTACCCGGCACCCCCAGGGCTACCACCTGTGGAGCGAAGCCATCCCTCTCTGTCAA
GCTCTTTCCTGTGGGCTTCCTGAGGCCCCCAAGAATGGAATGGTGTTTGGCAAGGAGTACACAGTGGG
AACCAAGGCCATGTACAGCTGCAGTGAAGGCTACCACCTCCAGGCAGGCGCTGAGGCCACTGCAGAGT
GTCTGGACACAGGCCTATGGAGCAACCGCAATGTCCCACCACAGTGTGTCCCTGTGACTTGTCCTGAT
GTCAGTAGCATCAGCGTGGAGCATGGCCGATGGAGGCTTATCTTTGAGACACAGTATCAGTTCCAGGC
CCAGCTGATGCTCATCTGTGACCCTGGCTACTACTATACTGGCCAAAGGGTCATCCGCTGTCAGGCCA
ATGGCAAATGGAGCCTCGGGGACTCTACGCCCACCTGCCGAATCATCTCCTGTGGAGAGCTCCCGATT
CCCCCCAATGGCCACCGCATCGGAACACTGTCTGTCTACGGGGCAACAGCCATCTTCTCCTGCAATTC
CGGATACACACTGGTGGGCTCCAGGGTGCGTGAGTGCATGGCCAATGGGCTCTGGAGTGGCTCTGAAG
TCCGCTGCCTTGCTGGACACTGTGGGACTCCTGAGCCCATTGTCAACGGACACATCAATGGGGAGAAC
TACAGCTACCGGGGCAGTGTGGTGTACCAATGCAATGCTGGCTTCCGCCTGATCGGCATGTCTGTGCG
CATCTGCCAGCAGGATCATCACTGGTCGGGCAAGACCCCTTTCTGTGTGCCAATTACCTGTGGACACC
CAGGCAACCCTGTCAACGGCCTCACCCAGGGTAACCAGTTTAACCTCAACGATGTGGTCAAGTTTGTT
TGCAACCCTGGGTATATGGCTGAGGGGGCTGCTAGGTCCCAATGCCTGGCCAGCGGGCAGTGGAGTGA
CATGCTGCCCACCTGCAGAATCATCAACTGTACAGATCCTGGACACCAAGAAAATAGTGTTCGTCAGG
TCCACGCCAGCGGCCCGCACAGGTTCAGCTTCGGCACCACTGTGTCTTACCGGTGCAACCACGGCTTC
TACCTCCTGGGCACCCCAGTGCTCAGCTGCCAGGGAGATGGCACATGGGACCGTCCCCGCCCCCAGTG
TCTCTGTAAGGTCGACC

| | | | |
|---|---|---|---|
| NOV4g, 273095147 | SEQ ID NO: 44 | 776 aa | MW at 84741.1kD |
| Protein Sequence | | | |

KLECPDPEPFANGIVRGAGYNVGQSVTFECLPGYQLTGHPVLTCQHGTNRNWDHSLPKCEVPCGGNIT
SSNGTVYSPGFPSPYSSSQDCVWLITVPIGHGVRLNLSLLQTEPSGDFITIWDGPQQTAPRLGVFTRS
MAKKTVQSSSNQVLLKFHRDAATGGIFAIAFSAYPLTKCPPPTILPNAEVVTENEEFNIGDIVRYRCL
PGFTLVGNEILTCKLGTYLQFEGPPPICEVHCPTNELLTDSTGVILSQSYPGSYPQFQTCSWLVRVEP
DYNISLTVEYFLSEKQYDEFEIFDGPSGQSPLLKALSGNYSAPLIVTSSSNSVYLRWSSDHAYNRKGF
KIRYSAPYCSLPRAPLHGFILGQTSTQPGGSIHFGCNAGYRLVGHSMAICTRHPQGYHLWSEAIPLCQ
ALSCGLPEAPKNGMVFGKEYTVGTKAMYSCSEGYHLQAGAEATAECLDTGLWSNRNVPPQCVPVTCPD
VSSISVEHGRWRLIFETQYQFQAQLMLICDPGYYYTGQRVIRCQANGKWSLGDSTPTCRIISCGELPI
PPNGGRIGTLSVYGATAIFSCNSGYTLVGSRVRECMANGLWSGSEVRCLAGHCGTPEPIVNGHINGEN
YSYRGSVVYQCNAGFRLIGMSVRICQQDHHWSGKTPFCVPITCGHPGNPVNGLTQGNQFNLNDVVKFV
CNPGYMAEGAARSQCLASGQWSDMLPTCRIINCTDPGHQENSVRQVHASGPHRFSFGTTVSYRCNHGF
YLLGTPVLSCQGDGTWDRPRPQCLCKVD

| | | |
|---|---|---|
| Nov4h, 317459653 | SEQ ID NO: 45 | 2328 bp |
| DNA Sequence | ORF Start: at 1 | ORF Stop: end of sequence |

AAGCTTGAGTGCCCAGACCCAGAGCCCTTTGCCAATGGCATTGTGAGGGGAGCTGGCTACAACGTGGG
ACAATCAGTGACCTTCGAGTGCCTCCCGGGGTATCAATTGACTGGCCACCCTGTCCTCACGTGTCAAC
ATGGCACCAACCGGAACTGGGACCACCCCCTGCCCAAGTGTGAAGTCCCTTGTGGCGGGAACATCACT
TCTTCCAACGGCACTGTGTACTCCCCGGGGTTCCCTAGCCCGTACTCCAGCTCCCAGGACTGTGTCTG
GCTGATCACCGTGCCCATTGGCCATGGCGTCCGCCTCAACCTCAGCCTGCTGCAGACAGAGCCCTCTG
GAGATTTCATCACCATCTGGGATGGGCCACAGCAAACAGCACCACGGCTCGGCGTCTTCACCCGGAGC
ATGGCCAAGAAAACAGTGCAGAGTTCATCCAACCAGGTCCTGCTCAAGTTCCACCGTGATGCAGCCAC
AGGGGGGATCTTCGCCATAGCTTTCTCCGCTTATCCACTCACCAAATGCCCTCCTCCCACCATCCTCC
CCAACGCCGAAGTCGTCACAGAGAATGAAGAATTCAATATAGGTGACATCGTACGCTACAGATGCCTC
CCTGGCTTTACCTTAGTGGGGAATGAAATTCTGACCTGCAAACTTGGAACCTACCTGCAGTTTGAAGG
ACCACCCCCGATATGTGAAGTGCACTGTCCAACAAATGAGCTTCTGACAGACTCCACAGGCGTGATCC
TGAGCCAGAGCTACCCTGGAAGCTATCCCCAGTTCCAGACCTGCTCTTGGCTGGTGAGAGTGGAGCCC
GACTATAACATCTCCCTCACAGTGGAGTACTTCCTCAGCGAGAAGCAATATGATGAGTTTGAGATTTT
TGATGGTCCATCAGGACAGAGTCCTCTGCTGAAAGCCCTCAGTGGGAATTACTCAGCTCCCCTGATTG
TCACCAGCTCAAGCAACTCTGTGTACCTGCGTTGGTCATCTGATCACGCCTACAATCGGAAGGGCTTC
AAGATCCGCTATTCAGCCCCTTACTGCAGCCTGCCCAGGGCTCCACTCCATGGCTTCATCCTAGGCCA
GACCAGCACCCAGCCCGGGGGCTCCATCCACTTTGGCTGCAACGCCGGCTACCGCCTGGTGGGACACA
GCATGGCCATCTGTACCCGGCACCCCCAGGGCTACCACCTGTGGAGCGAAGCCATCCCTCTCTGTCAA
GCTCTTTCCTGTGGGCTTCCTGAGGCCCCCAAGAATGGAATGGTGTTTGGCAAGGAGTACACAGTGGG
AACCAAGGCCGTGTACAGCTGCAGTGAAGGCTACCACCTCCAGGCAGGCGCTGAGGCCACTGCAGAGT
GTCTGGACACAGGCCTATGGAGCAACCGCAATGTCCCACCACAGTGTGTCCCTGTGACTTGTCCTGAT
GTCAGTAGCATCAGCGTGGAGCATGGCCGATGGAGGCTTATCTTTGAGACACAGTATCAGTTCCAGGC
CCAGCTGATGCTCATCTGTGACCCTGGCTACTACTATACTGGCCAAAGGGTCATCCGCTGTCAGGCCA
ATGGCAAATGGAGCCTCGGGGACTCTACGCCCACCTGCCGAATCATCTCCTGTGGAGAGCTCCCGATT
CCCCCCAATGGCCACCGCATCGGAACACTGTCTGTCTACGGGGCAACAGCCATCTTCTCCTGCAATTC
CGGATACACACTGGTGGGCTCCAGGGTGCGTGAGTGCATGGCCAATGGGCTCTGGAGTGGCTCTGAAG
TCCGCTGCCTTGCTGGACACTGTGGGACTCCTGAGCCCATTGTCAACGGACACATCAATGGGGAGAAC
TACAGCTACCGGGGCAGTGTGGTGTACCAATGCAATGCTGGCTTCCGCCTGATCGGCATGTCTGTGCG
CATCTGCCAGCAGGATCATCACTGGTCGGGCAAGACCCCTTTCTGTGTGCCAATTACCTGTGGACACC
CAGGCAACCCTGTCAACGGCCTCACTCAGGGTAACCAGTTTAACCTCAACGATGTGGTCAAGTTTGTT
TGCAACCCTGGGTATATGGCTGAGGGGGCTGCTAGGTCCCAATGCCTGGCCAGCGGGCAATGGAGTGA
CATGCTGCCCACCTGCAGAATCATCAACTGTACAGATCCTGGACACCAAGAAAATAGTGTTCGTCAGG
TCCACGCCAGCGGCCCGCACAGGTTCAGCTTCGGCACCACTGTGTCTTACCGGTGCAACCACGGCTTC
TACCTCCTGGGCACCCCAGTGCTCAGCTGCCAGGGAGATGGCACATGGGACCGTCCCCGCCCCCAGTG
TCTCTGTAAGGTCGAC

| | | | |
|---|---|---|---|
| NOV4h, 317459653 | SEQ ID NO: 46 | 776 aa | MW at 84719.1kD |
| Protein Sequence | | | |

KLECPDPEPFANGIVRGAGYNVGQSVTFECLPGYQLTGHPVLTCQHGTNRNWDHPLPKCEVPCGGNIT
SSNGTVYSPGFPSPYSSSQDCVWLITVPIGHGVRLNLSLLQTEPSGDFITIWDGPQQTAPRLGVFTRS
MAKKTVQSSSNQVLLKFHRDAATGGIFAIAFSAYPLTKCPPPTILPNAEVVTENEEFNIGDIVRYRCL
PGFTLVGNEILTCKLGTYLQFEGPPPICEVHCPTNELLTDSTGVILSQSYPGSYPQFQTCSWLVRVEP
DYNISLTVEYFLSEKQYDEFEIFDGPSGQSPLLKALSGNYSAPLIVTSSSNSVYLRWSSDHAYNRKGF
KIRYSAPYCSLPRAPLHGFILGQTSTQPGGSIHFGCNAGYRLVGHSMAICTRHPQGYHLWSEAIPLCQ
ALSCGLPEAPKNGMVFGKEYTVGTKAVYSCSEGYHLQAGAEATAECLDTGLWSNRNVPPQCVPVTCPD
VSSISVEHGRWRLIFETQYQFQAQLMLICDPGYYYTGQRVIRCQANGKWSLGDSTPTCRIISCGELPI
PPNGHRIGTLSVYGATAIFSCNSGYTLVGSRVRECMANGLWSGSEVRCLAGHCGTPEPIVNGHINGEN
YSYRGSVVYQCNAGFRLIGMSVRICQQDHHWSGKTPFCVPITCGHPGNPVNGLTQGNQFNLNDVVKFV
CNPGYMAEGAARSQCLASGQWSDMLPTCRIINCTDPGHQENSVRQVHASGPHRFSFGTTVSYRCNHGF
YLLGTPVLSCQGDGTWDRPRPQCLCKVD

| | | |
|---|---|---|
| NOV4i, 317459612 | SEQ ID NO: 47 | 2139 bp |
| DNA Sequence | ORF Start: at 1 | ORF Stop: end of sequence |

AAGCTTGAGTGCCCAGACCCAGAGCCCTTTGCCAATGGCATTGTGAGGGGAGCTGGCTACAACGTGGG
ACAATCAGTGACCTTCGAGTGCCTCCCGGGGTATCAATTGACTGGCCACCCTGTCCTCACGTGTCAAC
ATGGCACCAACCGGAACTGGGACCACCCCCTGCCCAAGTGTGAAGTCCCTTGTGGCGGGAACATCACT
TCTTCCAACGGCACTGTGTACTCCCCGGGGTTCCCTAGCCCGTACTCCAGCTCCCAGGACTGTGTCTG
GCTGATCACCGTGCCCATTGGCCATGGCGTCCGCCTCAACCTCAGCCTGCTGCAGACAGAGCCCTCTG
GAGATTTCATCACCATCTGGGATGGGCCACAGCAAACAGCACCACGGCTCGGCGTCTTCACCCGGAGC
ATGGCCAAGAAAACAGTGCAGAGTTCATCCAACCAGGTCCTGCTCAAGTTCCACCGTGATGCAGCCAC
AGGGGGGATCTTCGCCATAGCTTTCTCCGCTTATCCACTCACCAAATGCCCTCCTCCCACCATCCTCC
CCAACGCCGAAGTCGTCACAGAGAATGAAGAATTCAATATAGGTGACATCGTACGCTACAGATGCCTC
CCTGGCTTTACCTTAGTGGGGAATGAAATTCTGACCTGCAAACTTGGAACCTACCTGCAGTTTGAAGG
ACCACCCCCGATATGTGAAGTGCACTGTCCAACAAATGAGCTTCTGACAGACTCCACAGGCGTGATCC
TGAGCCAGAGCTACCCTGGAAGCTATCCCCAGTTCCAGACCTGCTCTTGGCTGGTGAGAGTGGAGCCC
GACTATAACATCTCCCTCACAGTGGAGTACTTCCTCAGCGAGAAGCAATATGATGAGTTTGAGATTTT
TGATGGTCCATCAGGACAGAGTCCTCTGCTGAAAGCCCTCAGTGGGAATTACTCAGCTCCCCTGATTG
TCACCAGCTCAAGCAACTCTGTGTACCTGCGTTGGTCATCTGATCACGCCTACAATCGGAAGGGCTTC
AAGATCCGCTATTCAGCTCTTTCCTGTGGGCTTCCTGAGGCCCCCAAGAATGGAATGGTGTTTGGCAA
GGAGTACACAGTGGGAACCAAGGCCATGTACAGCTGCAGTGAAGGCTACCACCTCCAGGCAGGCGCTG
AGGCCACTGCAGAGTGTCTGGACACAGGCCTATGGAGCAACCGCAATGTCCCACCACAGTGTGTCCCT
GTGACTTGTCCTGATGTCAGTAGCATCAGCGTGGAGCATGGCCGATGGAGGCCTATCTTTGAGACACA
GTATCAGTTCCAGGCCCAGCTGATGCTCATCTGTGACCCTGGCTACTACTATACTGGCCAAAGGGTCA
TCCGCTGTCAGGCCAATGGCAAATGGAGCCTCGGGGACTCTACGCCCACCTGCCGAATCATCTCCTGT
GGAGAGCTCCCGATTCCCCCCAATGGCCACCGCATCGGAACACTGTCTGTCTACGGGGCAACAGCCAT
CTTCTCCTGCAATTCCGGATACACACTGGTGGGCTCCAGGGTGCGTGAGTGCATGGCCAATGGGCTCT
GGAGTGGCTCTGAAGTCCGCTGCCTTGCTGGACACTGTGGGACTCCTGAGCCCATTGTCAACGGACAC
ATCAATGGGGAGAACTACAGCTACCGGGGCAGTGTGGTGTACCAATGCAATGCTGGCTTCCGCCTGAT
CGGCATGTCTGTGCGCATCTGCCAGCAGGATCATCACTGGTCGGGCAAGACCCCTTTCTGTGTGCCAA
TTACCTGTGGACACCCAGGCAACCCTGTCAACGGCCTCACTCAGGGTAACCAGTTTAACCTCAACGAT
GTGGTCAAGTTTGTTTGCAACCCTGGGTATATGGCTGAGGGGGCTGCTAGGTCCCAATGCCTGGCCAG
CGGGCAATGGAGTGACATGCTGCCCACCTGCAGAATCATCAACTGTACAGATCCTGGACACCAAGAAA
ATAGTGTTCGTCAGGTCCACGCCAGCGGCCCGCACAGGTTCAGCTTCGGCACCACTGTGTCTTACCGG
TGCAACCACGGCTTCTACCTCCTGGGCACCCCAGTGCTCAGCTGCCAGGGAGATGGCACATGGGACCG
TCCCCGCCCCCAGTGTCTCTGTAAGGTCGAC

| | | | |
|---|---|---|---|
| NOV4i, 317459612 | SEQ ID NO: 48 | 713 aa | MW at 77947.4kD |
| Protein Sequence | | | |

KLECPDPEPFANGIVRGAGYNVGQSVTFECLPGYQLTGHPVLTCQHGTNRNWDHPLPKCEVPCGGNIT
SSNGTVYSPGFPSPYSSSQDCVWLITVPIGHGVRLNLSLLQTEPSGDFTTIWDGPQQTAPRLGVFTRS
MAKKTVQSSSNQVLLKFHRDAATGGIFAIAFSAYPLTKCPPPTILPNAEVVTENEEFNIGDIVRYRCL
PGFTLVGNEILTCKLGTYLQFEGPPPICEVHCPTNELLTDSTGVILSQSYPGSYPQFQTCSWLVRVEP
DYNISLTVEYFLSEKQYDEFEIFDGPSGQSPLLKALSGNYSAPLIVTSSSNSVYLRWSSDHAYNRKGF
KIRYSALSCGLPEAPKNGMVFGKEYTVGTKAMYSCSEGYHLQAGAEATAECLDTGLWSNRNVPPQCVP
VTCPDVSSISVEHGRWRPIFETQYQFQAQLMLICDPGYYYTGQRVIRCQANGKWSLGDSTPTCRIISC
GELPIPPNGHRIGTLSVYGATAIFSCNSGYTLVGSRVRECMANGLWSGSEVRCLAGHCGTPEPIVNGH
INGENYSYRGSVVYQCNAGFRLIGMSVRICQQDHHWSGKTPFCVPITCGHPGNPVNGLTQGNQFNLND
VVKFVCNPGYMAEGAARSQCLASGQWSDMLPTCRIINCTDPGHQENSVRQVHASGPHRFSFGTTVSYR
CNHGFYLLGTPVLSCQGDGTWDRPRPQCLCKVD

| | | |
|---|---|---|
| NOV4j, 317286331 | SEQ ID NO: 49 | 2607 bp |
| DNA Sequence | ORF Start: at 1 | ORF Stop: end of sequence |

AAGCTTTCATGTTTTGATCCTGGTTCCATCAAGAACGGCACACGGGTGGGGTCCGACCTGAAGCTGGG
CTCCTCCGTCACCTACTACTGCCACGGGGGCTACGAAGTTGAGGGCACCTCGACCCTGAGCTGCATCC
TGGGGCCTGATGGGAAGCCCGTGTGGAACAATCCCCGGCCAGTCTGCACAGCCCCCTGTGGGGGACAG
TATGTGGGTTCGGACGGAGTGGTCTTGTCCCCCAACTACCCCCAGAACTACACCAGTGGACAGATCTG
CTTGTATTTTGTTACTGTGCCCAAGGACTATGTGGTGTTTGGCCAGTTCGCCTTCTTTCACACGGCCC
TCAACGACGTGGTGGAGGTTCACGACGGCCACAGCCAGCACTCGCGGCTCCTCAGCTCCCTCTCGGGC
TCCCATACAGGAGAATCACTGCCCTTGGCCACCTCCAATCAAGTTCTCATTAAGTTCAGCGCCAAAGG
CCTCGCACCAGCCAGAGGCTTCCACTTTGTCTACCAAGCGGTTCCTCGAACCAGCGCCACGCAGTGCA
GCTCTGTGCCGGAACCCCGCTATGGCAAGAGGCTGGGCAGTGACTTCTCGGTGGGGGCCATCGTCCGC
TTCGAATGCAACTCCGGCTATGCCCTGCAGGGGTCGCCAGAGATCGAGTGCCTCCCTGTGCCTGGGGC
CTTGGCCCAATGGAATGTCTCAGCGCCCACGTGTGTGGTGCCGTGTGGAGGCAACCTCACAGAGCGCA
GGGGCACCATCCTGTCCCCTGGCTTCCCAGAGCCGTACCTCAACAGCCTCAACTGTGTGTGGAAGATC
GTGGTCCCCGAAGGCGCTGGCATCCAGATCCAAGTTGTCAGTTTTGTGACAGAGCAGAACTGGGACTC
GCTGGAAGTATTTGATGGTGCAGATAACACTGTAACCATGCTGGGGAGTTTCTCAGGAACAACCGTGC
CTGCCCTTCTGAACAGCACCTCCAACCAGCTCTACCTTCATTTCTACTCAGATATCAGCGTATCTGCA
GCTGGCTTCCACTTGGAGTACAAAACGGTGGGCCTGAGCAGTTGTCCGGAACCTGCTGTGCCCAGTAA
CGGGGTGAAGACTGGCGAGCGCTACTTGGTGAATGATGTGGTGTCTTTCCAGTGTGAGCCGGGATATG
CCCTCCAGGGCCACGCCCACATCTCCTGCATGCCCGGAACAGTGCGGCGATGGAACTACCCTCCTCCA
CTCTGTATTGCACAGTGTGGGGGAACAGTGGAGGAGATGGAGGGGGTGATCCTGAGCCCCGGCTTCCC
AGGCAACTACCCCAGTAACATGGACTGCTCCTGGAAAATAGCACTGCCCGTGGGCTTTGGAGCTCACA
TCCAGTTCCTGAACTTCTCCACCGAGCCCAACCACGACTACATAGAAATCCGGAATGGCCCCTATGAG
ACCAGCCGCATGATGGGAAGATTCAGTGGAAGCGAGCTTCCAAGCTCCCTCCTCTCCACGTCCCACGA
GACCACCGTGTATTTCCACAGCGACCACTCCCAGAATCGGCCAGGATTCAAGCTGGAGTATCAGGCCT
ATGAACTTCAAGAGTGCCCAGACCCAGAGCCCTTTGCCAATGGCATTGTGAGGGGAGCTGGCTACAAC
GTGGGACAATCAGTGACCTTCGAGTGCCTCCCGGGGTATCAATTGACTGGCCACCCTGTCCTCACGTG
TCAACATGGCACCAACCGGAACTGGGACCACCCCCTGCCCAAGTGTGAAGTCCCTTGTGGCGGGAACA
TCACTTCTTCCAACGGCACTGTGTACTCCCCGGGGTTCCCTAGCCCGTACTCCAGCTCCCAGGACTGT
GTCTGGCTGATCACCGTGCCCATTGGCCATGGCGTCCGCCTCAACCTCAGCCTGCTGCAGACAGAGCC
CTCTGGAGATTTCATCACCATCTGGGATGGGCCACAGCAAACAGCACCACGGCTCGGCGTCTTCACCC
GGAGCATGGCCAAGAAAACAGTGCAGAGTTCATCCAACCAGGTCCTGCTCAAGTTCCACCGTGATGCA
GCCACAGGGGGGATCTTCGCCATAGCTTTCTCCGCTTATCCACTCACCAAATGCCCTCCTCCCACCAT
CCTCCCCAACGCCGAAGTCGTCACAGAGAATGAAGAATTCAATATAGGTGACATCGTACGCTACAGAT
GCCTCCCTGGCTTTACCTTAGTGGGGAATGAAATTCTGACCTGCAAACTTGGAACCTACCTGCAGTTT
GAAGGACCACCCCCGATATGTGAAGTGCACTGTCCAACAAATGAGCTTCTGACAGACTCCACAGGCGT
GATCCTGAGCCAGAGCTACCCTGGAAGCTATCCCCAGTTCCAGACCTGCTCTTGGCTGGTGAGAGTGG
AGCCCGACTATAACATCTCCCTCACAGTGGAGTACTTCCTCAGCGAGAAGCAATATGATGAGTTTGAG
ATTTTTGATGGTCCATCAGGACAGAGTCCTCTGCTGAAAGCCCTCAGTGGGAATTACTCAGCTCCCCT
GATTGTCACCAGCTCAAGCAACTCTGTGTACCTGCGTTGGTCATCTGATCACGCCTACAATCGGAAGG
GCTTCAAGATCCGCTATGTCGAC

| | | | |
|---|---|---|---|
| NOVv4j, 317286331 | SEQ ID NO: 50 | 869 aa | MW at 94553.5kD |
| Protein Sequence | | | |

KLSCFDPGSIKNGTRVGSDLKLGSSVTYYCHGGYEVEGTSTLSCILGPDGKPVWNNPRPVCTAPCGGQ
YVGSDGVVLSPNYPQNYTSGQICLYFVTVPKDYVVFGQFAFFHTALNDVVEVHDGHSQHSRLLSSLSG
SHTGESLPLATSNQVLIKFSAKGLAPARGFHFVYQAVPRTSATQCSSVPEPRYGKRLGSDFSVGAIVR
FECNSGYALQGSPEIECLPVPGALAQWNVSAPTCVVPCGGNLTERRGTILSPGFPEPYLNSLNCVWKI
VVPEGAGIQIQVVSFVTEQNWDSLEVFDGADNTVTMLGSFSGTTVPALLNSTSNQLYLHFYSDISVSA
AGFHLEYKTVGLSSCPEPAVPSNGVKTGERYLVNDVVSFQCEPGYALQGHAHISCMPGTVRRWNYPPP
LCIAQCGGTVEEMEGVILSPGFPGNYPSNMDCSWKIALPVGFGAHIQFLNFSTEPNHDYIEIRNGPYE
TSRMMGRFSGSELPSSLLSTSHETTVYFHSDHSQNRPGFKLEYQAYELQECPDPEPFANGIVRGAGYN
VGQSVTFECLPGYQLTGHPVLTCQHGTNRNWDHPLPKCEVPCGGNITSSNGTVYSPGFPSPYSSSQDC
VWLITVPIGHGVRLNLSLLQTEPSGDFITIWDGPQQTAPRLGVFTRSMAKKTVQSSSNQVLLKFHRDA
ATGGIFAIAFSAYPLTKCPPPTILPNAEVVTENEEFNIGDIVRYRCLPGFTLVGNEILTCKLGTYLQF
EGPPPICEVHCPTNELLTDSTGVILSQSYPGSYPQFQTCSWLVRVEPDYNISLTVEYFLSEKQYDEFE
IFDGPSGQSPLLKALSGNYSAPLIVTSSSNSVYLRWSSDHAYNRKGFKIRYVD

| | | |
|---|---|---|
| NOV4k, CG50377-07 | SEQ ID NO: 51 | 300 bp |
| DNA Sequence | ORF Start: at 2 | ORF Stop: at 299 |

AAAGTCATCTGTCTCCTTCTCCTGCCATCCCCCTCTGGTGCTGGTGGGCTCTCCACGCAGGTTTTGCC
AGTCAGATGGGACATGGAGTGGCACCCAGCCCAGCTGCATAGATCCGACCCTGACCACGTGTGCGGAC
CCTGGTGTGCCACAGTTTGGGATACAGAACAATTCTCAGGGCTACCAGGTTGGAAGCACAGTCCTCTT
CCGTTGTCAAAAAGGCTACCTGCTTCAGGGCTCCACCACCAGGACCTGCCTCCCAAACCTGACCTGGA
GTGGAACCCCACCTGACTGTGTGCCCCA

| | | | |
|---|---|---|---|
| NOV4k, CG50377-07 | SEQ ID NO: 52 | 99 aa | MW at 10565.8kD |
| Protein Sequence | | | |

KSSVSFSCHPPLVLVGSPRRFCQSDGTWSGTQPSCIDPTLTTCADPGVPQFGIQNNSQGYQVGSTVLF
RCQKGYLLQSTTRTCLPNLTWSGTPPDCVP

| | | |
|---|---|---|
| NOV4l, CG50377-08 | SEQ ID NO: 53 | 1792 bp |
| DNA Sequence | ORF Start: at 1 | ORF Stop: at 1792 |

GCTGAGGGGGCTGCTAGGTCCCAATGCCTGGCCAGCGGGCAGTGGAGTGACATGCTGCCCACCTGCAG
AATCATCAACTGTACAGATCCTGGACACCAAGAAAATAGTGTTCGTCAGGTCCACGCCAGCGGCCCGC
ACAGGTTCAGCTTCGGCACCACTGTGTCTTACCGGTGCAACCACGGCTTCTACCTCCTGGGCACCCCA
GTGCTCAGCTGCCAGGGAGATGGCACATGGGACCGTCCCCGCCCCCAGTGTCTCTTGGTGTCCTGTGG
CCATCCGGGCTCCCCGCCTCACTCCCAGATGTCTGGAGACAGTTATACTGTGGGAGCAGTGGTGCGGT
ACAGCTGCATCGGCAAGCGTACTCTGGTGGGAAACAGCACCCGCATGTGTGGGCTGGATGGACACTGG
ACTGGCTCCCTCCCTCACTGCTCAGGAACCAGCGTGGGAGTTTGCGGTGACCCTGGGATCCCGGCTCA
TGGCATCCGTTTGGGGGACAGCTTTGATCCAGGCACTGTGATGCGCTTCAGCTGTGAAGCTGGCCACG
TGCTCCGGGGATCGTCAGAGCGCACCTGTCAAGCCAATGGCTCGTGGAGCGGCTCGCAGCCTGAGTGT
GGAGTGATCTCTTGTGGGAACCCTGGGACTCCAAGTAATGCCCGAGTTGTGTTCAGTGATGGCCTGGT
TTTCTCCAGCTCTATCGTCTATGAGTGCCGGGAAGGATACTACGCCACAGGCCTGCTCAGCCGTCACT
GCTCGGTCAATGGTACCTGGACAGGCAGTGACCCTGAGTGCCTCGTCATAAACTGTGGTGACCCTGGG
ATTCCAGCCAATGGCCTTCGGCTGGGCAATGACTTCAGGTACAACAAAACTGTGACATATCAGTGTGT
CCCTGGCTATATGATGGAGTCACATAGAGTATCTGTGCTGAGCTGCACCAAGGACCGGACATGGAATG
GAACCAAGCCCGTCTGCAAAGCTCTCATGTGCAAGCCACCTCCGCTCATCCCCAATGGGAAGGTGGTG
GGGTCTGACTTCATGTGGGGCTCAAGTGTGACTTATGCCTGCCTGGAGGGGTACCAGCTCTCCCTGCC
CGCGGTGTTCACCTGTGAGGGAAATGGGTCCTGGACCGGAGAGCTGCCTCAGTGTTTCCCTGTGTTCT
GCGGGGATCCTGGTGTCCCGTCCCGTGGGAGGAGAGAGGACCGAGGCTTCTCCTACAGGTCATCTGTC
TCCTTCTCCTGCCATCCCCCTCTGGTGCTGGTGGGCTCTCCACGCAGGTTTTGCCAGTCAGATGGGAC
ATGGAGTGGCACCCAGCCCAGCTGCATAGATCCGACCCTGACCACGTGTGCGGACCCTGGTGTGCCAC
AGTTTGGGATACAGAACAATTCTCAGGGCTACCAGGTTGGAAGCACAGTCCTCTTCCGTTGTCAAAAA
GGCTACCTGCTTCAGGGCTCCACCACCAGGACCTGCCTCCCAAACCTGACCTGGAGTGGAACCCCACC
TGACTGTGTCCCCCACCACTGCAGGCAGCCAGAGACGCCAACGCATGCCAACGTCGGGGCCCTGGATT
TGCCCTCCATGGGCTACACGCTCATCTACTCCTGCCAGGAGGGCTTCTCCCTCAAGGGTGGCTCCGAG
CACCGCACCTGCAAGGCGGATGGCAGCTGGACAGGCAAGCCGCCCATCTGCCTGGAGGTCCGGCCCAG
TGGGAGACCCATCAACATTGCCCGGGAGCCACCGCTCACCCAAGCCTTGATTCCTGGGGATGTTTTTG
CCAAGAATTCCCTGTGGAAAGGGG

| | | | |
|---|---|---|---|
| NOV4l, CG50377-08 | SEQ ID NO: 54 | 597 aa | MW at 63914.5kD |
| Protein Sequence | | | |

AEGAARSQCLASGQWSDMLPTCRIINCTDPGHQENSVRQVHASGPHRFSFGTTVSYRCNHGFYLLGTP
VLSCQGDGTWDRPRPQCLLVSCGHPGSPPHSQMSGDSYTVGAVVRYSCIGKRTLVGNSTRMCGLDGHW
TGSLPHCSGTSVGVCGDPGIPAHGIRLGDSFDPGTVMRFSCEAGHVLRGSSERTCQANGSWSGSQPEC
GVISCGNPGTPSNARVVFSDGLVFSSSIVYECREGYYATGLLSRHCSVNGTWTGSDPECLVINCGDPG
IPANGLRLGNDFRYNKTVTYQCVPGYMMESHRVSVLSCTKDRTWNGTKPVCKALNCKPPPLIPNGKVV
GSDFMWGSSVTYACLEGYQLSLPAVFTCEGNGSWTGELPQCFPVFCGDPGVPSRGRREDRGFSYRSSV
SFSCHPPLVLVGSPRRFCQSDGTWSGTQPSCIDPTLTTCADPGVPQFGIQNNSQGYQVGSTVLFRCQK
GYLLQGSTTRTCLPNLTWSGTPPDCVPHHCRQPETPTHANVGALDLPSMGYTLIYSCQEGFSLKGGSE
HRTCKADGSWTGKPPICLEVRPSGRPINIAREPPLTQALIPGDVFAKNSLWKG

| | | |
|---|---|---|
| NOV4m, CG50377-09 | SEQ ID NO: 55 | 1135 bp |
| DNA Sequence | ORF Start: ATG at 510 | ORF Stop: TAG at 1044 |

TTAAGAACCAGGTGTCGCTGTCCCTCCGCTCCTGCACATTTCTATTTGGTATTTCAATATGTGACATG
AATCTTGAGGCCAGCATGTCACACGTATACAGGGGCTTAAGCTTGTGGGGGCCTTCCCGAGGACAGAG
AAAGTGGCCCTAAATGCTATACTTTGATTCCTAAACTCATGAACTTCCCAACAATTCTCATGGAATTC
ATGAACGACCCAAGCATGATGGGACATGGAGTGGCACCCAGCCCAGCTGCATAGATCCGACCCTGACC
ACGTGTGCGGACCCTGGTGTGCCACAGTTTGGGATACAGAACAATTCTCAGGGCTACCAGGTTGGAAG
CACAGTCCTCTTCCGTTGTCAAAAAGGCTACCTGCTTCAGGGCTCCACCACCAGGACCTGCCTCCCAA
ACCTGACCTGGAGTGGAACCCCACCTGACTGTGTCCCCCACCACTGCAGGCAGCCAGAGACGCCAACG
CATGCCAACGTCGGGGCCCTGGATTTGCCCTCCATGGGCTACACGCTCATCTACTCCTGCCAGGAGGG
CTTCTCCCTCAAGGGTGGCTCCGAGCACCGCACCTGCAAGGCGGATGGCAGCTGGACAGGCAAGCCGC
CCATCTGCCTGGAGGTCCGGCCCAGTGGGAGACCCATCAACACTGCCCGGGAGCCACCGCTCACCCAA
GCCTTGATTCCTGGGGATGTTTTTGCCAAGAATTCCCTGTGGAAAGGGGCCTATGAATACCAGGGGAA
GAAGCAGCCAGCCATGCTCAGAGTGACTGGCTTCCAAGTTGCCAACAGCAAGGTCAATGCCACCATGA
TCGACCACAGTGGCGTGGAGCTGCACTTGGCTGGAACTTACAAGAAAGAAGATTTTCATCTCCTACTC
CAGGTGTACCAGATTACAGGGCCTGTGGAGATCTTTATGAATAAGTTCAAAGATGATCACTGGGCTTT
AGATGGCCATGTAAGTTCAGCAAACTCACCTCCTCCCCTGACTCCCAGATCACTGGCAGGGAAGAGGA
GGACAAAAGGGTGTGCAAACCCCTAGGATCAATCTGGCAACTTGGGAGGAGGTTTTCTGTGGGGCCCT
GCCTAGGAGCAGCAAGAGGATCCTCATCTGCTAATGAAATAGGCAAA

| | | | |
|---|---|---|---|
| NOV4m, CG50377-09 | SEQ ID NO: 56 | 178 aa | MW at 19569.2kD |
| Protein Sequence | | | |

MGYTLIYSCQEGFSLKGGSEHRTCKADGSWTGKPPICLEVRPSGRPINTAREPPLTQALIPGDVFAKN
SLWKGAYEYQGKKQPAMLRVTGFQVANSKVNATMIDHSGVELHLAGTYKKEDFHLLLQVYQITGPVEI
FMNKFKDDHWALDGHVSSANSPPPLTPRSLAGKRRTKGCANP

| | | |
|---|---|---|
| NOV4n, CG50377-10 | SEQ ID NO: 57 | 3905 bp |
| DNA Sequence | ORF Start: ATG at 427 | ORF Stop: TGA at 3079 |

GGATTTGAGAGCCTGAACTTAGCCATACACCAGATCTACCTTTGGACCGCAAAAGGGACCCAGTGCTT
CATGAAGCTGGTTTTTTTTGTTTTGTTTTGTTTTTTTTCCGTTGTTTTGTTTCGGCTTTACCAACCTG
ACTGGGTGTTTTTCAATATCCACCATTCAGACTTTCCTCAACAGCAGAGGATGTGGCAGTGGCAAAGA
CAAGGGGATGGGGGGAGACGAAAGGGAAAGGGGCCTGCATGAAAGACCATGTCTGTCTTCCTGCTGGT
GCCAGTTCCCTGAACCTCATCTTGTTGTTCAGCCCCTTACTGCAGCCTGCCCAGGGCTCCACTCCATG
GCTTCATCCTAGGCCAGACCAGCACCCAGCCCGGGGGCTCCATCCACTTTGGCTGCAACGCCGGCTAC
CGCCTGGTGGGACACAGCATGGCCATCTGTACCCGGCACCCCCAGGGCTACCACCTGTGGAGCGAAGC
CATCCCTCTCTGTCAAGCTCTTTCCTGTGGGCTTCCTGAGGCCCCCAAGAATGGAJJGGTGTTTGGCA
AGGAGTACACAGTGGGAACCAAGGCCGTGTACAGCTGCAGTGAAGGCTACCACCTCCAGGCAGGCGCT
GAGGCCACTGCAGAGTGTCTGGACACAGGCCTATGGAGCAACCGCAATGTCCCACCACAGTGTGTCCC
TGTGACTTGTCCTGATGTCAGTAGCATCAGCGTGGAGCATGGCCGATGGAGGCTTATCTTTGAGACAC
AGTATCAGTTCCAGGCCCAGCTGATGCTCATCTGTGACCCTGGCTACTACTATACTGGCCAAAGGGTC
ATCCGCTGTCAGGCCAATGGCAAATGGAGCCTCGGGGACTCTACGCCCACCTGCCGAATCATCTCCTG
TGGAGAGCTCCCGATTCCCCCCAATGGCCACCGCATCGGAACACTGTCTGTCTACGGGGCAACAGCCA
TCTTCTCCTGCAATTCCGGATACACACTGGTGGGCTCCAGGGTGCGTGAGTGCATGGCCAATGGGCTC
TGGAGTGGCTCTGAAGTCCGCTGCCTTGCTGGACACTGTGGGACTCCTGAGCCCATTGTCAACGGACA
CATCAATGGGGAGAACTACAGCTACCGGGGCAGTGTGGTGTACCAATGCAATGCTGGCTTCCGCCTGA
TCGGCATGTCTGTGCGCATCTGCCAGCAGGATCATCACTGGTCGGGCAAGACCCCTTTCTGTGTGCCA
ATTACCTGTGGACACCCAGGCAACCCTGTCAACGGCCTCACTCAGGGTAACCAGTTTAACCTCAACGA
TGTGGTCAAGTTTGTTTGCAACCCTGGGTATATGGCTGAGGGGGCTGCTAGGTCCCAATGCCTGGCCA
GCGGGCAATGGAGTGACATGCTGCCCACCTGCAGAATCATCAACTGTACAGATCCTGGACACCAAGAA
AATAGTGTTCGTCAGGTCCACGCCAGCGGCCCGCACAGGTTCAGCTTCGGCACCACTGTGTCTTACCG
GTGCNNAACCACGGCTTCTACCTCCTGGGCAACCCCAGTGCTCAGCTGCCAGGGAGATGGCACATGGG
ACCGTCCCCGCCCCCAGTGTCTCTTGGTGTCCTGTGGCCATCCGGGCTCCCCGCCTCACTCCCAGATG
TCTGGAGACAGTTATACTGTGGGAGCAGTGGTGCGGTACAGCTGCATCGGCAAGCGTACTCTGGTGGG
AAACAGCACCCGCATGTGTGGGCTGGATGGACACTGGACTGGCTCCCTCCCTCACTGCTCAGGAACCA
GCGTGGGAGTTTGCGGTGACCCTGGGATCCCGGCTCATGGCATCCGTTTGGGGGACAGCTTTGATCCA
GGCACTGTGATGCGCTTCAGCTGTGAAGCTGGCCACGTGCTCCGGGGATCGTCAGAGCGCACCTGTCA
AGCCAATGGCTCGTGGAGCGGCTCGCAGCCTGAGTGTGGAGTGATCTCTTGTGGGAACCCTGGGACTC
CAAGTAATGCCCGAGTTGTGTTCAGTGATGGCCTGGTTTTCTCCAGCTCTATCGTCTATGAGTGCCGG
GAAGGATACTACGCCACAGGCCTGCTCAGCCGTCACTGCTCGGTCAATGGTACCTGGACAGGCAGTGA
CCCTGAGTGCCTCGTCATAAACTGTGGTGACCCTGGGATTCCAGCCAATGGCCTTCGGCTGGGCAATG
ACTTCAGGTACAACAAAACTGTGACATATCAGTGTGTCCCTGGCTATATGATGGAGTCACATAGAGTA
TCTGTGCTGAGCTGCACCAAGGACCGGACATGGAATGGAACCAAGCCCGTCTGCAAAGCTCTCATGTG
CAAGCCACCTCCGCTCATCCCCAATGGGAAGGTGGTGGGGTCTGACTTCATGTGGGGCTCAAGTGTGA
CTTATGCCTGCCTGGAGGGGTACCAGCTCTCCCTGCCCGCGGTGTTCACCTGTGAGGGAAATGGGTCC
TGGACCGGAGAGCTGCCTCAGTGTTTCCCTGTGTTCTGCGGGGATCCTGGTGTCCCGTCCCGTGGGAG
GAGAGAGGACCGAGGCTTCTCCTACAGGTCATCTGTCTCCTTCTCCTGCCATCCCCCTCTGGTGCTGG
TGGGCTCTCCACGCAGGTTTTGCCAGTCAGATGGGACATGGAGTGGCACCCAGCCCAGCTGCATAGAT
CCGACCCTGACCACGTGTGCGGACCCTGGTGTGCCACAGTTTGGGATACAGAACAATTCTCAGGGCTA
CCAGGTTGGAAGCACAGTCCTCTTCCGTTGTCAAAAAGGCTACCTGCTTCAGGGCTCCACCACCAGGA
CCTGCCTCCCAAACCTGACCTGGAGTGGAACCCCACCTGACTGTGTCCCCCACCACTGCAGGCAGCCA
GAGACGCCAACGCATGCCAACGTCGGGGCCCTGGATTTGCCCTCCATGGGCTACACGCTCATTACTCC
TGCCAGGAGGGCTTCTCCCTCAAGGGTGGCTCCGAGCACCGCACCTGCAAGGCGGATGGCAGCTGGAC
AGGCAAGCCGCCCATCTGCCTGGCAGAGGTCCGGCCCAGTGGGAGACCCATCAACACTGCCCGGGAGC
CACCGCTCACCCAAGCCTTGATTCCTGGGGATGTTTTTGCCAAGAATTCCCTGTGGAAAGGGGCCTAT
GAATACCAGGGGAAGAAGCAGCCAGCCATGCTCAGAGTGACTGGCTTCCAAGTTGCCAACAGCAAGGT
CAATGCCACCATGATCGACCACAGTGGCGTGGAGCTGCACTTGGCTGGAACTTACAAGAAAGAAGATT
TTCATCTCCTACTCCAGGTGTACCAGATTACAGGGCCTGTGGAGATCTTTATGAATAAGTTCAAAGAT
GATCACTGGGCTTTAGATGGCCATGTCTCGTCAGAGTCCTCCGGAGCCACCTTCATCTACCAAGGCTC
TGTCAAGGGCCAAGGCTTTGGGCAGTTCGGCTTTCAAAGACTGGACCTCAGGCTGCTGGAGTCAGACC
CCGAGTCCATTGGCCGCCACTTTGCTTCCAACAGCAGCTCAGTGGCAGCCGCGATCCTGGTGCCTTTC
ATCGCCCTCATTATTGCGGGCTTCGTGCTCTATCTCTACAAGCACAGGAGAAGACCCAAAGTTCCTTT
CAATGGCTATGCTGGCCAcGAGAACACCAATGTTCGGGCCACATTTGAGAACCCAATGTACGACCGCA
ACATCCAGCCCACAGACATCATGGCCAGCGAGGCGGAGTTCACAGTCAGCACAGTGTGCACAGCAGTA
TAGCCACCCGGCCTGGCCGCTTTTTTTGCTAGGTTGAACTGGTACTCCAGCAGCCGCCGAAGCTGGAC
TGTACTGCTGCCATCTCAGCTCACTGCAACCTCCCTGCCTGATTCCCCTGCCTCAGCCTGCCGAGTGC
CTGCGATTGCAGGCGCGCACCGCCACNNT

| | | | |
|---|---|---|---|
| NOV4n, CG50377-10 | SEQ ID NO: 58 | 884 aa | MW at 94718.1kD |
| Protein Sequence | | | |

MAICTRHPQGYHLWSEAIPLCQALSCGLPEAPKNGMVFGKEYTVGTKAVYSCSEGYHLQAGAEATAEC
LDTGLWSNRNVPPQCVPVTCPDVSSISVEHGRWRLIFETQYQFQAQLMLICDPGYYYTGQRVIRCQAN
GKWSLGDSTPTCRIISCGELPIPPNGHRIGTLSVYGATAIFSCNSGYTLVGSRVRECMANGLWSGSEV
RCLAGHCGTPEPIVNGHINGENYSYRGSVVYQCNAGFRLIGMSVRICQQDHHWSGKTPFCVPITCGHP
GNPVNGLTQGNQFNLNDVVKFVCNPGYMAEGAARSQCLASGQWSDMLPTCRIINCTDPGHQENSVRQV
HASGPHRFSFGTTVSYRCXTTASTSWATPVLSCQGDGTWDRPRPQCLLVSCGHPGSPPHSQMSGDSYT
VGAVVRYSCIGKRTLVGNSTRMCGLDGHWTGSLPHCSGTSVGVCGDPGIPAHGTRLGDSFDPGTVMRF
SCEAGHVLRGSSERTCQANGSWSGSQPECGVISCGNPGTPSNARVVFSDGLVFSSSIVYECREGYYAT
GLLSRHCSVNGTWTGSDPECLVINCGDPGIPANGLRLGNDFRYNKTVTYQCVPGYMMESHRVSVLSCT
KDRTWNGTKPVCKALMCKPPPLIPNGKVVGSDFMWGSSVTYACLEGYQLSLPAVFTCEGNGSWTGELP
QCFPVFCGDPGVPSRGRPEDRGFSYRSSVSFSCHPPLVLVGSPRRFCQSDGTWSGTQPSCIDPTLTTC
ADPGVPQFGIQNNSQGYQVGSTVLFRCQKGYLLQGSTTRTCLPNLTWSGTPPDCVPHHCRQPETPTHA
NVGALDLPSMGYTLITPARRASPSRVAPSTAPARRMAAGQASRPSAWQRSGPVGDPSTLPGSHRSPKP

| | | |
|---|---|---|
| NOV4o, CG50377-11 | SEQ ID NO: 59 | 3896 bp |
| DNA Sequence | ORF Start: ATG at 427 | ORF Stop: TGA at 3073 |

GGATTTGAGAGCCTGAACTTAGCCATACACCAGATCTACCTTTGGACCGCAAAAGGGACCCAGTGCTT
CATGAAGCTGGTTTTTTTTGTTTTGTTTTGTTTTTTTTCCGTTGTTTTGTTTCGGCTTTACCAACCTG
ACTGGGTGTTTTTCAATATCCACCATTCAGACTTTCCTCAACAGCAGAGGATGTGGCAGTGGCAAAGA
CAAGGGGATGGGGGGAGACGAAAGGGAAAGGGGCCTGCATGAAAGACCATGTCTGTCTTCCTGCTGGT
GCCAGTTCCCTGAACCTCATCTTGTTGTTCAGCCCCTTACTGCAGCCTGCCCAGGGCTCCACTCCATG
GCTTCATCCTAGGCCAGACCAGCACCCAGCCCGGGGGCTCCATCCACTTTGGCTGCAACGCCGGCTAC
CGCCTGGTGGGACACAGCATGGCCATCTGTACCCGGCACCCCCAGGGCTACCACCTGTGGAGCGAAGC
CATCCCTCTCTGTCAAGCTCTTTCCTGTGGGCTTCCTGAGGCCCCCAAGAATGGAATGGTGTTTGGCA
AGGAGTACACAGTGGGAACCAAGGCCGTGTACAGCTGCAGTGAAGGCTACCACCTCCAGGCAGGCGCT
GAGGCCACTGCAGAGTGTCTGGACACAGGCCTATGGAGCAACCGCAATGTCCCACCACAGTGTGTCCC
TGTGACTTGTCCTGATGTCAGTAGCATCAGCGTGGAGCATGGCCGATGGAGGCTTATCTTTGAGACAC
AGTATCAGTTCCAGGCCCAGCTGATGCTCATCTGTGACCCTGGCTACTACTATACTGGCCAAAGGGTC
ATCCGCTGTCAGGCCAATGGCAAATGGAGCCTCGGGGACTCTACGCCCACCTGCCGAATCATCTCCTG
TGGAGAGCTCCCGATTCCCCCCAATGGCCACCGCATCGGAACACTGTCTGTCTACGGGGCAACAGCCA
TCTTCTCCTGCAATTCCGGATACACACTGGTGGGCTCCAGGGTGCGTGAGTGCATGGCCAATGGGCTC
TGGAGTGGCTCTGAAGTCCGCTGCCTTGCTGGACACTGTGGGACTCCTGAGCCCATTGTCAACGGACA
CATCAATGGGGAGAACTACAGCTACCGGGGCAGTGTGGTGTACCAATGCAATGCTGGCTTCCGCCTGA
TCGGCATGTCTGTGCGCATCTGCCAGCAGGATCATCACTGGTCGGGCAAGACCCCTTTCTGTGTGCCA
ATTACCTGTGGACACCCAGGCAACCCTGTCAACGGCCTCACTCAGGGTAACCAGTTTAACCTCAACGA
TGTGGTCAAGTTTGTTTGCAACCCTGGGTATATGGCTGAGGGGGCTGCTAGGTCCCAATGCCTGGCCA
GCGGGCAATGGAGTGACATGCTGCCCACCTGCAGAATCATCAACTGTACAGATCCTGGACACCAAGAA
AATAGTGTTCGTCAGGTCCACGCCAGCGGCCCGCACAGGTTCAGCTTCGGCACCACTGTGTCTTACCG
GTGCAACCACGGCTTCTACCTCCTGGGCACCCCAGTGCTCAGCTGCCAGGGAGATGGCACATGGGACC
GTCCCCGCCCCCAGTGTCTCTTGGTGTCCTGTGGCCATCCGGGCTCCCCGCCTCACTCCCAGATGTCT
GGAGACAGTTATACTGTGGGAGCAGTGGTGCGGTACAGCTGCATCGGCAAGCGTACTCTGGTGGGAAA
CAGCACCCGCATGTGTGGGCTGGATGGACACTGGACTGGCTCCCTCCCTCACTGCTCAGGAACCAGCG
TGGGAGTTTGCGGTGACCCTGGGATCCCGGCTCATGGCATCCGTTTGGGGGACAGCTTTGATCCAGGC
ACTGTGATGCGCTTCAGCTGTGAAGCTGGCCACGTGCTCCGGGGATCGTCAGAGCGCACCTGTCAAGC
CAATGGCTCGTGGAGCGGCTCGCAGCCTGAGTGTGGAGTGATCTCTTGTGGGAACCCTGGGACTCCAA
GTAATGCCCGAGTTGTGTTCAGTGATGGCCTGGTTTTCTCCAGCTCTATCGTCTATGAGTGCCGGGAA
GGATACTACGCCACAGGCCTGCTCAGCCGTCACTGCTCGGTCAATGGTACCTGGACAGGCAGTGACCC
TGAGTGCCTCGTCATAAACTGTGGTGACCCTGGGATTCCAGCCAATGGCCTTCGGCTGGGCAATGACT
TCAGGTACAACAAAACTGTGACATATCAGTGTGTCCCTGGCTATATGATGGAGTCACATAGAGTATCT
GTGCTGAGCTGCACCAAGGACCGGACATGGAATGGAACCAAGCCCGTCTGCAAAGCTCTCATGTGCAA
GCCACCTCCGCTCATCCCCAATGGGAAGGTGGTGGGGTCTGACTTCATGTGGGGCTCAAGTGTGACTT
ATGCCTGCCTGGAGGGGTACCAGCTCTCCCTGCCCGCGGTGTTCACCTGTGAGGGAAATGGGTCCTGG
ACCGGAGAGCTGCCTCAGTGTTTCCCTGTGTTCTGCGGGGATCCTGGTGTCCCGTCCCGTGGGAGGAG
AGAGGACCGAGGCTTCTCCTACAGGTCATCTGTCTCCTTCTCCTGCCATCCCCCTCTGGTGCTGGTGG
GCTCTCCACGCAGGTTTTGCCAGTCAGATGGGACATGGAGTGGCACCCAGCCCAGCTGCATAGATCCG
ACCCTGACCACGTGTGCGGACCCTGGTGTGCCACAGTTTGGGATACAGAACAATTCTCAGGGCTACCA
GGTTGGAAGCACAGTCCTCTTCCGTTGTCAAAAAGGCTACCTGCTTCAGGGCTCCACCACCAGGACCT
GCCTCCCAAACCTGACCTGGAGTGGAACCCCACCTGACTGTGTCCCCCACCACTGCAGGCAGCCAGAG
ACGCCAACGCATGCCAACGTCGGGGCCCTGGATTTGCCCTCCATGGGCTACACGCTCATTACTCCTGC
CAGGAGGGCTTCTCCCTCAAGGGTGGCTCCGAGCACCGCACCTGCAAGGCGGATGGCAGCTGGACAGG
CAAGCCGCCCATCTGCCTGGAGGTCCGGCCCAGTGGGAGACCCATCAACACTGCCCGGGAGCCACCGC
TCACCCAAGCCTTGATTCCTGGGGATGTTTTTGCCAAGAATTCCCTGTGGAAAGGGGCCTATGAATAC
CAGGGGAAGAAGCAGCCAGCCATGCTCAGAGTGACTGGCTTCCAAGTTGCCAACAGCAAGGTCAATGC
CACCATGATCGACCACAGTGGCGTGGAGCTGCACTTGGCTGGAACTTACAAGAAAGAAGATTTTCATC
TCCTACTCCAGGTGTACCAGATTACAGGGCCTGTGGAGATCTTTATGAATAAGTTCAAAGATGATCAC
TGGGCTTTAGATGGCCATGTCTCGTCAGAGTCCTCCGGAGCCACCTTCATCTACCAAGGCTCTGTCAA
GGGCCAAGGCTTTGGGCAGTTCGGCTTTCAAAGACTGGACCTCAGGCTGCTGGAGTCAGACCCCGAGT
CCATTGGCCGCCACTTTGCTTCCAACAGCAGCTCAGTGGCAGCCGCGATCCTGGTGCCTTTCATCGCC
CTCATTATTGCGGGCTTCGTGCTCTATCTCTACAAGCACAGGAGAAGACCCAAAGTTCCTTTCAATGG
CTATGCTGGCCACGAGAACACCAATGTTCGGGCCACATTTGAGAACCCAATGTACGACCGCAACATCC
AGCCCACAGACATCATGGCCAGCGAGGCGGAGTTCACAGTCAGCACAGTGTGCACAGCAGTATAGCCA
CCCGGCCTGGCCGCTTTTTTTGCTAGGTTGAACTGGTACTCCAGCAGCCGCCCAAGCTGGACTGTACT
GCTGCCATCTCAGCTCACTGCAACCTCCCTGCCTGATTCCCCTGCCTCAGCCTGCCGAGTGCCTGCGA
TTGCAGGCGCGCACCGCCAC

| | | |
|---|---|---|
| NOV4o, CG50377-11 | SEQ ID NO: 60 | 882 aa MW at 94686.1kD |
| Protein Sequence | | |

MAICTRHPQGYHLWSEAIPLCQALSCGLPEAPKNGMVFGKEYTVGTKAVYSCSEGYHLQAGAEATAEC
LDTGLWSNRNVPPQCVPVTCPDVSSISVEHGRWRLIFETQYQFQAQLMLICDPGYYYTGQRVIRCQAN
GKWSLGDSTPTCRIISCGELPIPPNGHRIGTLSVYGATAIFSCNSGYTLVGSRVRECMANGLWSGSEV
RCLAGHCGTPEPIVNGHINGENYSYRGSVVYQCNAGFRLIGMSVRICQQDHHWSGKTPFCVPITCGHP
GNPVNGLTQGNQFNLNDVVKFVCNPGYMAEGAARSQCLASGQWSDMLPTCRIINCTDPGHQENSVRQV
HASGPHRFSFGTTVSYRCNHGFYLLGTPVLSCQGDGTWDRPRPQCLLVSCGHPGSPPHSQMSGDSYTV
GAVVRYSCIGKRTLVGNSTRMCGLDGHWTGSLPHCSGTSVGVCGDPGIPAHGTRLGDSFDPGTVMRFS
CEAGHVLRGSSERTCQANGSWSGSQPECGVISCGNPGTPSNARVVFSDGLVFSSSIVYECREGYYATG
LLSRHCSVNGTWTGSDPECLVINCGDPGIPANGLRLGNDFRYNKTVTYQCVPGYMMESHRVSVLSCTK
DRTWNGTKPVCKALMCKPPPLIPNGKVVGSDFMWGSSVTYACLEGYQLSLPAVFTCEGNGSWTGELPQ
CFPVFCGDPGVPSRGRREDRGFSYRSSVSFSCHPPLVLVGSPRRFCQSDGTWSGTQPSCIDPTLTTCA
DPGVPQFGIQNNSQGYQVGSTVLFRCQKGYLLQGSTTRTCLPNLTWSGTPPDCVPHHCRQPETPTHAN
VGALDLPSMGYTLITPARRASPSRVAPSTAPARRMAAGQASRPSAWRSGPVGDPSTLPGSHRSPKP

| | | |
|---|---|---|
| NOV4p, SNP13382457 of | SEQ ID NO: 61 | 10136 bp |
| CG50377-01, DNA Sequence | ORF Start: ATG at 1 | ORF Stop: TGA at 9313 |
| | SNP Pos: 3120 | SNP Change: G to A |

ATGGCGGGCGCCCCTCCCCCCGCCTTGCTGCTGCCTTGCAGTTTGATCTCAGACTGCTGTGCTAGCAA
TCAGCGACACTCCGTGGGCGTAGGACCCTCCGAGCTAGTCAAGAAGCAAATTGAGTTGAAGTCTCGAG
GTGTGAAGCTGATGCCCAGCAAAGACAACAGCCAGAAGACGTCTGTGTTAACTCAGGTTGGTGTGTCC
CAAGGACATAATATGTGTCCAGACCCTGGCATACCCGAAAGGGGCAAAAGACTAGGCTCGGATTTCAG
GTTAGGATCCAGCGTCCAGTTCACCTGCAACGAGGGCTATGACCTGCAAGGGTCCAAGCGGATCACCT
GTATGAAAGTGAGCGACATGTTTGCGGCCTGGAGCGACCACAGGCCAGTCTGCCGAGCCCGCATGTGT
GATGCCCACCTTCGAGGCCCCTCGGGCATCATCACCTCCCCCAATTTCCCCATTCAGTATGACAACAA
TGCACACTGTGTGTGGATCATCACAGCACTCAACCCCTCCAAGGTGATCAAGCTCGCCTTTGAGGAGT
TTGATTTGGAGAGGGGCTATGACACCCTGACGGTCGGTGATGGTGGTCAGGATGGGGACCAGAAGACA
GTTCTCTACATGTCTCAAAATGCCTGCAGTGACAGCCCTCACACCCCAGGCTCTCGCATCCCAGAGAG
CATGTCTGGGGACATCTGGAGGCAGAAATGGACTGTACTTGAGATCTGTCGTGACATTAGCAGTTCAG
ATGCAAGGTCAGGTTCAGTGAGGAAGTCTCCAAAGACTTCTAATGCTGTGGAACTTGTTGCTCCTGGG
ACAGAGATCGAGCAGGGCAGTTGCGGTGACCCTGGCATACCTGCATATGGCCGGAGGGAAGGCTCCCG
GTTTCACCACGGTGACACACTCAAGTTTGAGTGCCAGCCCGCCTTTGAGCTGGTGGGACAGAAGGCAA
TCACATGCCAAAAGAATAACCAATGGTCGGCTAAGAAGCCAGGCTGCGTGTTCTCCTGCTTCTTCAAC
TTCACCAGCCCGTCTGGGGTTGTCCTGTCTCCCAACTACCCAGAGGACTATGGCAACCACCTCCACTG
TGTCTGGCTCATCCTGGCCAGGCCTGAGAGCCGCATCCACCTGGCCTTCAACGACATTGACGTGGAGC
CTCAGTTTGATTTCCTGGTCATCAAGGATGGGGCCACCGCCGAGGCGCCCGTCCTGGGCACCTTCTCA
GGAAACCAGCTTCCCTCCTCCATCACAAGCAGTGGCCACGTGGCCCGTCTCGAGTTCCAGACTGACCA
CTCCACAGGGAAGAGGGGCTTCAACATCACTTTTACCACCTTCCGACACAACGAGTGCCCGGATCCTG
GCGTTCCAGTAAATGGCAAACGGTTTGGGGACAGCCTCCAGCTGGGCAGCTCCATCTCCTTCCTCTGT
GATGAAGGCTTCCTTGGGACTCAGGGCTCAGAGACCATCACCTGCGTCCTGAAGGAGGGCAGCGTGGT
CTGGAACAGCGCTGTGCTGCGGTGTGAAGCTCCCTGTGGTGGTCACCTGACTTCGCCCAGCGGCACCA
TCCTCTCTCCGGGCTGGCCTGGCTTCTACAAGGATGCCTTGAGCTGTGCCTGGGTGATTGAGGCCCAG
CCAGGCTACCCCATCAAAATCACCTTCGACAGATTCAAAACCGAGGTCAACTATGACACCCTGGAAGT
ACGCGATGGGCGGACTTACTCAGCGCCCTTGATCGGGGTTTACCACGGGACCCAGGTTCCCCAGTTCC
TCATCAGCACCAGCAACTACCTCTACCTCCTCTTCTCTACCGACAAGAGTCACTCGGACATCGGCTTC
CAGCTCCGCTATGAGACTATAACACTGCAGTCAGACCACTGTCTGGATCCAGGAATCCCAGTAAATGG
ACAGCGTCATGGGAATGACTTCTACGTGGGCGCGCTGGTGACCTTCAGCTGTGACTCGGGCTACACAT
TAAGTGACGGGGAGCCTCTGGAGTGTGAGCCCAACTTCCAGTGGAGCCGGGCCCTGCCCAGTTGTGAA
GCTCTCTGTGGTGGCTTCATTCAAGGCTCCAGTGGGACCATCTTGTCGCCAGGGTTCCCTGACTTCTA
CCCCAACAACTTGAACTGCACCTGGATTATCGAAACATCTCATGGCAAGGGTGTGTTCTTCACTTTCC
ACACCTTCCACCTGGAAAGTGGCCATGACTACCTCCTCATCACTGAGAACGGCAGCTTCACCCAGCCC
CTGAGGCAGCTAACTGGATCTCGGCTGCCAGCTCCCATCAGCGCTGGGCTCTATGGCAACTTCACTGC
CCAGGTCCGCTTCATCTCTGATTTCTCCATGTCATATGAAGGATTCAACATCACCTTCTCAGAGTACG
ACTTGGAGCCCTGTGAGGAGCCCGAGGTCCCAGCCTACAGCATCCGGAAGGGCTTGCAGTTTGGCGTG
GGCGACACCTTGACCTTCTCCTGCTTCCCCGGGTACCGTCTGGAGGGCACCGCCCGCATCACGTGCCT
GGGGGGCAGACGGCGCCTGTGGAGCTCGCCTCTGCCAAGGTGTGTTGCTGAGTGTGGGAATTCAGTCA
CAGGCACTCAGGGTACTTTGCTGTCCCCCAACTTTCCTGTGAACTACAATAACAATCATGAATGCATC
TACTCCATCCAGACCCAGCCAGGGAAGGGAATTCAGCTGAAAGCCAGGGCATTCGAACTCTCCGAAGG
AGATGTCCTCAAGGTTTATGATGGCAACAACAACTCCGCCCGTTTGCTGGGAGTTTTTAGCCATTCTG
AGATGATGGGGGTGACTTTGAACAGCACATCCAGCAGTCTGTGGCTTGATTTCATCACTGATGCTGAA
AACACCAGCAAGGGCTTTGAACTGCACTTTTCCAGCTTTGAACTCATCAAATGTGAGGACCCAGGAAC
CCCCAAGTTTGGCTACAAGGTTCATGATGAAGGTCATTTTGCAGGGAGCTCCGTGTCCTTCAGCTGTG
ACCCTGGATACAGCCTGCGGGGTAGTGAGGAGCTGCTGTGTCTGAGTGGAGAGCGCCGGACCTGGGAC
CGGCCTCTGCCCACCTGTGTCGCCGAGTGTGGAGGGACAGTGAGAGGAGAGGTGTCGGGACAGGTGCT
GTCACCCGGGTATCCAGCTCCCTATGAACACAATCTCAACTGCATCTGGACCATCGAAGCAGAGGCCG
GCTGCACCATTGGGCTACACTTCCTGGTGTTTGACACAGAGGAGGTTCACGACGTGCTGCGCATCTGG
GATGGGCCTGTGGAGAGCGGGGTTCTGCTGAAGGAGCTGAGTGGCCCGGCCCTGCCCAAGGACCTGCA
TAGCACCTTCAACTCGGTCGTCCTGCAGTTCAGCACTGACTTCTTCACCAGCAAGCAGGGCTTTGCCA
TTCAATTTTCAGTGTCCACAGCAACGTCCTGCAATGACCCTGGGATCCCGCAGAATGGGAGTCGGAGT
GGTGACAGTTGGGAAGCCGGCGACTCCACAGTGTTCCAGTGTGACCCTGGCTACGCGCTGCAGGGAAG
TGCAGAGATCAGCTGTGTGAAGATCGAGAACAGGTTCTTCTGGCAGCCCAGCCCGCCAACATGCATCG
CTCCCTGCGGGGGAGACCTGACAGGACCATCTGGAGTCATCCTCTCACCAAATTACCCAGAACCCTAC
CCGCCAGGCAAGGAGTGTGACTGGAAAGTGACCGTCTCACCAGACTACGTCATCGCCCTGGTATTTAA
CATCTTTAACCTGGAGCCTGGCTATGACTTCCTCCATATCTACGACGGACGGGACTCTCTCAGCCCTC
TCATAGGAAGCTTCTATGGCTCCCAGCTCCCAGGCCGCATTGAAAGCAGCAGCAACAGCCTCTTCCTC
GCCTTCCGCAGCGATGCATCTGTGAGCAATGCTGGCTTCGTCATTGACTATACAGAAAACCCGCGGGA
GTCATGTTTTGATCCTGGTTCCATCAAGAACGGCACACGGGTGGGGTCCGACCTGAAGCTGGGCTCCT
CCGTCACCTACTACTGCCACGGGGGCTACGAAGTTGAGGGCACCTCGACCCTGAGCTGCATCCTGGGG
CCTGATGGGAAGCCCGTGTGGAACAATCCCCGGCCAGTCTGCACAGCCCCCTGTGGGGGACAGTATGT
GGGTTCGGACGGAGTGGTCTTGTCCCCCAACTACCCCCAGAACTACACCAGTGGACAGATCTGCTTGT
ATTTTGTTACTGTGCCCAAGGACTATGTGGTGTTTGGCCAGTTCGCCTTCTTTCACACGGCCCTCAAC
GACGTGGTGGAGGTTCACGACGGCCACAGCCAGCACTCGCGGCTCCTCAGCTCCCTCTCGGGCTCCCA
TACAGGAGAATCACTGCCCTTGGCCACCTCCAATCAAGTTCTCATTAAGTTCAGCGCCAAAGGCCTCG
CACCAGCCAGAGGCTTCCACTTTGTCTACCAAGCGGTTCCTCGAACCAGCGCCACGCAGTGCAGCTCT
GTGCCGGAACCCCGCTATGGCAAGAGGCTGGGCAGTGACTTCTCGGTGGGGGCCATCGTCCGCTTCGA
ATGCAACTCCGGCTATGCCCTGCAGGGGTCGCCAGAGATCGAGTGCCTCCCTGTGCCTGGGGCCTTGG
CCCAATGGAATGTCTCAGCGCCCACGTGTGTGGTGCCGTGTGGAGGCAACCTCACAGAGCGCAGGGGC
ACCATCCTGTCCCCTGGCTTCCCAGAGCCGTACCTCAACAGCCTCAACTGTGTGTGGAAGATCGTGGT
CCCCGAAGGCGCTGGCATCCAGATCCAAGTTGTCAGTTTTGTGACAGAGCAGAACTGGGACTCGCTGG
AAGTATTTGATGGTGCAGATAACACTGTAACCATGCTGGGGAGTTTCTCAGGAACAACCGTGCCTGCC
CTTCTGAACAGCACCTCCAACCAGCTCTACCTTCATTTCTACTCAGATATCAGCGTATCTGCAGCTGG
CTTCCACTTGGAGTACAAAACGGTGGGCCTGAGCAGTTGTCCGGAACCTGCTGTGCCCAGTAACGGGG
TGAAGACTGGCGAGCGCTACTTGGTGAATGATGTGGTGTCTTTCCAGTGTGAGCCGGGATATGCCCTC
CAGGGCCACGCCCACATCTCCTGCATGCCCGGAACAGTGCGGCGATGGAACTACCCTCCTCCACTCTG
TATTGCACAGTGTGGGGGAACAGTGGAGGAGATGGAGGGGGTGATCCTGAGCCCCGGCTTCCCAGGCA
ACTACCCCAGTAACATGGACTGCTCCTGGAAAATAGCACTGCCCGTGGGCTTTGGAGCTCACATCCAG
TTCCTGAACTTCTCCACCGAGCCCAACCACGACTACATAGAAATCCGGAATGGCCCCTATGAGACCAG
CCGCATGATGGGAAGATTCAGTGGAAGCGAGCTTCCAAGCTCCCTCCTCTCCACGTCCCACGAGACCA
CCGTGTATTTCCACAGCGACCACTCCCAGAATCGGCCAGGATTCAAGCTGGAGTATCAGGCCTATGAA
CTTCAAGAGTGCCCAGACCCAGAGCCCTTTGCCAATGGCATTGTGAGGGGAGCTGGCTACAACGTGGG
ACAATCAGTGACCTTCGAGTGCCTCCCGGGGTATCAATTGACTGGCCACCCTGTCCTCACGTGTCAAC
ATGGCACCAACCGGAACTGGGACCACCCCCTGCCCAAGTGTGAAGTCCCTTGTGGCGGGAACATCACT
TCTTCCAACGGCACTGTGTACTCCCCGGGGTTCCCTAGCCCGTACTCCAGCTCCCAGGACTGTGTCTG
GCTGATCACCGTGCCCATTGGCCATGGCGTCCGCCTCAACCTCAGCCTGCTGCAGACAGAGCCCTCTG
GAGATTTCATCACCATCTGGGATGGGCCACAGCAAACAGCACCACGGCTCGGCGTCTTCACCCGGAGC
ATGGCCAAGAAAACAGTGCAGAGTTCATCCAACCAGGTCCTGCTCAAGTTCCACCGTGATGCAGCCAC
AGGGGGGATCTTCGCCATAGCTTTCTCCGCTTATCCACTCACCAAATGCCCTCCTCCCACCATCCTCC
CCAACGCCGAAGTCGTCACAGAGAATGAAGAATTCAATATAGGTGACATCGTACGCTACAGATGCCTC
CCTGGCTTTACCTTAGTGGGGAATGAAATTCTGACCTGCAAACTTGGAACCTACCTGCAGTTTGAAGG
ACCACCCCCGATATGTGAAGTGCACTGTCCAACAAATGAGCTTCTGACAGACTCCACAGGCGTGATCC
TGAGCCAGAGCTACCCTGGAAGCTATCCCCAGTTCCAGACCTGCTCTTGGCTGGTGAGAGTGGAGCCC
GACTATAACATCTCCCTCACAGTGGAGTACTTCCTCAGCGAGAAGCAATATGATGAGTTTGAGATTTT
TGATGGTCCATCAGGACAGAGTCCTCTGCTGAAAGCCCTCAGTGGGAATTACTCAGCTCCCCTGATTG
TCACCAGCTCAAGCAACTCTGTGTACCTGCGTTGGTCATCTGATCACGCCTACAATCGGAAGGGCTTC
AAGATCCGCTATTCAGCCCCTTACTGCAGCCTGCCCAGGGCTCCACTCCATGGCTTCATCCTAGGCCA
GACCAGCACCCAGCCCGGGGGCTCCATCCACTTTGGCTGCAACGCCGGCTACCGCCTGGTGGGACACA
GCATGGCCATCTGTACCCGGCACCCCCAGGGCTACCACCTGTGGAGCGAAGCCATCCCTCTCTGTCAA
GCTCTTTCCTGTGGGCTTCCTGAGGCCCCCAAGAATGGAATGGTGTTTGGCAAGGAGTACACAGTGGG
AACCAAGGCCGTGTACAGCTGCAGTGAAGGCTACCACCTCCAGGCAGGCGCTGAGGCCACTGCAGAGT
GTCTGGACACAGGCCTATGGAGCAACCGCAATGTCCCACCACAGTGTGTCCCTGTGACTTGTCCTGAT
GTCAGTAGCATCAGCGTGGAGCATGGCCGATGGAGGCTTATCTTTGAGACACAGTATCAGTTCCAGGC
CCAGCTGATGCTCATCTGTGACCCTGGCTACTACTATACTGGCCAAAGGGTCATCCGCTGTCAGGCCA
ATGGCAAATGGAGCCTCGGGGACTCTACGCCCACCTGCCGAATCATCTCCTGTGGAGAGCTCCCGATT
CCCCCCAATGGCCACCGCATCGGAACACTGTCTGTCTACGGGGCAACAGCCATCTTCTCCTGCAATTC
CGGATACACACTGGTGGGCTCCAGGGTGCGTGAGTGCATGGCCAATGGGCTCTGGAGTGGCTCTGAAG
TCCGCTGCCTTGCTGGACACTGTGGGACTCCTGAGCCCATTGTCAACGGACACATCAATGGGGAGAAC
TACAGCTACCGGGGCAGTGTGGTGTACCAATGCAATGCTGGCTTCCGCCTGATCGGCATGTCTGTGCG
CATCTGCCAGCAGGATCATCACTGGTCGGGCAAGACCCCTTTCTGTGTGCCAATTACCTGTGGACACC
CAGGCAACCCTGTCAACGGCCTCACTCAGGGTAACCAGTTTAACCTCAACGATGTGGTCAAGTTTGTT
TGCAACCCTGGGTATATGGCTGAGGGGGCTGCTAGGTCCCAATGCCTGGCCAGCGGGCAATGGAGTGA
CATGCTGCCCACCTGCAGAATCATCAACTGTACAGATCCTGGACACCAAGAAAATAGTGTTCGTCAGG
TCCACGCCAGCGGCCCGCACAGGTTCAGCTTCGGCACCACTGTGTCTTACCGGTGCAACCACGGCTTC
TACCTCCTGGGCACCCCAGTGCTCAGCTGCCAGGGAGATGGCACATGGGACCGTCCCCGCCCCCAGTG
TCTCTTGGTGTCCTGTGGCCATCCGGGCTCCCCGCCTCACTCCCAGATGTCTGGAGACAGTTATACTG
TGGGAGCAGTGGTGCGGTACAGCTGCATCGGCAAGCGTACTCTGGTGGGAAACAGCACCCGCATGTGT
GGGCTGGATGGACACTGGACTGGCTCCCTCCCTCACTGCTCAGGAACCAGCGTGGGAGTTTGCGGTGA
CCCTGGGATCCCGGCTCATGGCATCCGTTTGGGGGACAGCTTTGATCCAGGCACTGTGATGCGCTTCA
GCTGTGAAGCTGGCCACGTGCTCCGGGGATCGTCAGAGCGCACCTGTCAAGCCAATGGCTCGTGGAGC
GGCTCGCAGCCTGAGTGTGGAGTGATCTCTTGTGGGAACCCTGGGACTCCAAGTAATGCCCGAGTTGT
GTTCAGTGATGGCCTGGTTTTCTCCAGCTCTATCGTCTATGAGTGCCGGGAAGGATACTACGCCACAG
GCCTGCTCAGCCGTCACTGCTCGGTCAATGGTACCTGGACAGGCAGTGACCCTGAGTGCCTCGTCATA
AACTGTGGTGACCCTGGGATTCCAGCCAATGGCCTTCGGCTGGGCAATGACTTCAGGTACAACAAAAC
TGTGACATATCAGTGTGTCCCTGGCTATATGATGGAGTCACATAGAGTATCTGTGCTGAGCTGCACCA
AGGACCGGACATGGAATGGAACCAAGCCCGTCTGCAAAGCTCTCATGTGCAAGCCACCTCCGCTCATC
CCCAATGGGAAGGTGGTGGGGTCTGACTTCATGTGGGGCTCAAGTGTGACTTATGCCTGCCTGGAGGG
GTACCAGCTCTCCCTGCCCGCGGTGTTCACCTGTGAGGGAAATGGGTCCTGGACCGGAGAGCTGCCTC
AGTGTTTCCCTGTGTTCTGCGGGGATCCTGGTGTCCCGTCCCGTGGGAGGAGAGAGGACCGAGGCTTC
TCCTACAGGTCATCTGTCTCCTTCTCCTGCCATCCCCCTCTGGTGCTGGTGGGCTCTCCACGCAGGTT
TTGCCAGTCAGATGGGACATGGAGTGGCACCCAGCCCAGCTGCATAGATCCGACCCTGACCACGTGTG
CGGACCCTGGTGTGCCACAGTTTGGGATACAGAACAATTCTCAGGGCTACCAGGTTGGAAGCACAGTC
CTCTTCCGTTGTCAAAAAGGCTACCTGCTTCAGGGCTCCACCACCAGGACCTGCCTCCCAAACCTGAC
CTGGAGTGGAACCCCACCTGACTGTGTCCCCCACCACTGCAGGCAGCCAGAGACGCCAACGCATGCCA
ACGTCGGGGCCCTGGATTTGCCCTCCATGGGCTACACGCTCATTACTCCTGCCAGGAGGGCTTCTCCC
TCAAGGGTGGCTCCGAGCACCGCACCTGCAAGGCGGATGGCAGCTGGACAGGCAAGCCGCCCATCTGC
CTGGAGGTCCGGCCCAGTGGGAGACCCATCAACACTGCCCGGGAGCCACCGCTCACCCAAGCCTTGAT
TCCTGGGGATGTTTTTGCCAAGAATTCCCTGTGGAAAGGGGCCTATGAATACCAGGGGAAGAAGCAGC
CAGCCATGCTCAGAGTGACTGGCTTCCAAGTTGCCAACAGCAAGGTCAATGCCACCATGATCGACCAC
AGTGGCGTGGAGCTGCACTTGGCTGGAACTTACAAGAAAGAAGATTTTCATCTCCTACTCCAGGTGTA
CCAGATTACAGGGCCTGTGGAGATCTTTATGAATAAGTTCAAAGATGATCACTGGGCTTTAGATGGCC
ATGTCTCGTCAGAGTCCTCCGGAGCCACCTTCATCTACCAAGGCTCTGTCAAGGGCCAAGGCTTTGGG
CAGTTCGGCTTTCAAAGACTGGACCTCAGGCTGCTGGAGTCAGACCCCGAGTCCATTGGCCGCCACTT
TGCTTCCAACAGCAGCTCAGTGGCAGCCGCGATCCTGGTGCCTTTCATCGCCCTCATTATTGCGGGCT
TCGTGCTCTATCTCTACAAGCACAGGAGAAGACCCAAAGTTCCTTTCAATGGCTATGCTGGCCACGAG
AACACCAATGTTCGGGCCACATTTGAGAACCCAATGTACGACCGCAACATCCAGCCCACAGACATCAT
GGCCAGCGAGGCGGAGTTCACAGTCAGCACAGTGTGCACAGCAGTATAGCCACCCGGCCTGGCCGCTT
TTTTTGCTAGGTTGAACTGGTACTCCAGCAGCCGCCGAAGCTGGACTGTACTGCTGCCATCTCAGCTC
CCAC

| | | | |
|---|---|---|---|
| NOV4p, SNP13382457 of | SEQ ID NO: 62 | 3104 aa | MW at 336746.3kD |
| CG50377-01, | SNP Pos: 1040 | | SNP Change: Gly to Gly |
| Protein Sequence | | | |

MAGAPPPALLLPCSLISDCCASNQRHSVGVGPSELVKKQIELKSRGVKLMPSKDNSQKTSVLTQVGVS
QGHNMCPDPGIPERGKRLGSDFRLGSSVQFTCNEGyDLQGSKRITCMKVSDMFAAWSDHRPVCRARMC
DAHLRGPSGIITSPNFPIQYDNNAHCVWIITALNPSKVIKLAFEEFDLERGYDTLTVGDGGQDGDQKT
VLYMSQNACSDSPHTPGSRIPESMSGDIWRQKWTVLEICRDISSSDARSGSVRKSPKTSNAVELVAPG
TEIEQGSCGDPGIPAYGRREGSRFHHGDTLKFECQPAFELVGQKAITCQKNNQWSAKKPGCVFSCFFN
FTSPSGVVLSPNYPEDYGNHLHCVWLILARPESRIHLAFNDIDVEPQFDFLVIKDGATAEAPVLGTFS
GNQLPSSITSSGHVARLEFQTDHSTGKRGFNITFTTFRHNECPDPGVPVNGKRFGDSLQLGSSISFLC
DEGFLGTQGSETITCVLKEGSVVWNSAVLRCEAPCGGHLTSPSGTILSPGWPGFYKDALSCAWVIEAQ
PGYPIKITFDRFKTEVNYDTLEVRDGRTYSAPLIGVYHGTQVPQFLISTSNYLYLLFSTDKSHSDIGF
QLRYETITLQSDHCLDPGIPVNGQRHGNDFYVGALVTFSCDSGYTLSDGEPLECEPNFQWSRALPSCE
ALCGGFIQGSSGTILSPGFPDFYPNNLNCTWIIETSHGKGVFFTFHTFHLESGHDYLLITENGSFTQP
LRQLTGSRLPAPISAGLYGNFTAQVRFISDFSMSYEGFNITFSEYDLEPCEEPEVPAYSIRKGLQFGV
GDTLTFSCFPGYRLEGTARITCLGGRRRLWSSPLPRCVAECGNSVTGTQGTLLSPNFPVNYNNNHECI
YSIQTQPGKGIQLKAPAFELSEGDVLKVYDGNNNSARLLGVFSHSEMMGVTLNSTSSSLWLDFITDAE
NTSKGFELHFSSFELIKCEDPGTPKFGYKVHDEGHFAGSSVSFSCDPGYSLRGSEELLCLSGERRTWD
RPLPTCVAECGGTVRGEVSGQVLSPGYPAPYEHNLNCIWTIEAEAGCTIGLHFLVFDTEEVHDVLRIW
DGPVESGVLLKELSGPALPKDLHSTFNSVVLQFSTDFFTSKQGFAIQFSVSTATSCNDPGIPQNGSRS
GDSWEAGDSTVFQCDPGYALQGSAEISCVKIENRFFWQPSPPTCIAPCGGDLTGPSGVILSPNYPEPY
PPGKECDWKVTVSPDYVIALVFNIFNLEPGYDFLHIYDGRDSLSPLIGSFYGSQLPGRIESSSNSLFL
AFRSDASVSNAGFVIDYTENPRESCFDPGSIKNGTRVGSDLKLGSSVTYYCHGGYEVEGTSTLSCILG
PDGKPVWNNPRPVCTAPCGGQYVGSDGVVLSPNYPQNYTSGQICLYFVTVPKDYVVFGQFAFFHTALN
DVVEVHDGHSQHSRLLSSLSGSHTGESLPLATSNQVLIKFSAKGLAPARGFHFVYQAVPRTSATQCSS
VPEPRYGKRLGSDFSVGAIVRFECNSGYALQGSPEIECLPVPGALAQWNVSAPTCVVPCGGNLTERRG
TILSPGFPEPYLNSLNCVWKIVVPEGAGIQIQVVSFVTEQNWDSLEVFDGADNTVTMLGSFSGTTVPA
LLNSTSNQLYLHFYSDISVSAAGFHLEYKTVGLSSCPEPAVPSNGVKTGERYLVNDVVSFQCEPGYAL
QGHAHISCMPGTVRRWNYPPPLCIAQCGGTVEEMEGVILSPGFPGNYPSNMDCSWKIALPVGFGAHIQ
FLNFSTEPNHDYIEIRNGPYETSRMMGRFSGSELPSSLLSTSHETTVYFHSDHSQNRPGFKLEYQAYE
LQECPDPEPFANGIVRGAGYNVGQSVTFECLPGYQLTGHPVLTCQHGTNRNWDHPLPKCEVPCGGNIT
SSNGTVYSPGFPSPYSSSQDCVWLITVPIGNGVRLNLSLLQTEPSGDFITIWDGPQQTAPRLGVFTRS
MAKKTVQSSSNQVLLKFHRDAATGGIFAIAFSAYPLTKCPPPTILPNAEVVTENEEFNIGDIVRYRCL
PGFTLVGNEILTCKLGTYLQFEGPPPICEVHCPTNELLTDSTGVILSQSYPGSYPQFQTCSWLVRVEP
DYNISLTVEYFLSEKQYDEFEIFDGPSGQSPLLKALSGNYSAPLIVTSSSNSVYLRWSSDHAYNRKGF
KIRYSAPYCSLPPAPLHGFILGQTSTQPGGSIHFGCNAGYRLVGHSMAICTRHPQGYHLWSEAIPLCQ
ALSCGLPEAPKNGMVFGKEYTVGTKAVYSCSEGYHLQAGAEATAECLDTGLWSNRNVPPQCVPVTCPD
VSSISVEHGRWRLIFETQYQFQAQLMLICDPGYYYTGQRVIRCQANGKWSLGDSTPTCRIISCGELPI
PPNGHRIGTLSVYGATAIFSCNSGYTLVGSRVRECMANGLWSGSEVRCLAGHCGTPEPIVNGHINGEN
YSYRGSVVYQCNAGFRLIGMSVRICQQDHHWSGKTPFCVPITCGHPGNPVNGLTQGNQFNLNDVVKFV
CNPGYMAEGAARSQCLASGQWSDMLPTCRIINCTDPGHQENSVRQVHASGPHRFSFGTTVSYRCNHGF
YLLGTPVLSCQGDGTWDRPRPQCLLVSCGHPGSPPHSQMSGDSYTVGAVVRYSCIGKRTLVGNSTRMC
GLDGHWTGSLPHCSGTSVGVCGDPGIPAHGIRLGDSFDPGTVMRFSCEAGHVLRGSSERTCQANGSWS
GSQPECGVISCGNPGTPSNARVVFSDGLVFSSSIVYECREGYYATGLLSRHCSVNGTWTGSDPECLVI
NCGDPGIPANGLRLGNDFRYNKTVTYQCVPGYMMESHRVSVLSCTKDRTWNGTKPVCKALMCKPPPLI
PNGKVVGSDFMWGSSVTYACLEGYQLSLPAVFTCEGNGSWTGELPQCFPVFCGDPGVPSRGRPEDRGF
SYRSSVSFSCHPPLVLVGSPRRFCQSDGTWSGTQPSCIDPTLTTCADPGVPQFGIQNNSQGYQVGSTV
LFRCQKGYLLQGSTTRTCLPNLTWSGTPPDCVPHHCRQPETPTHANVGALDLPSMGYTLITPARRASP
SRVAPSTAPARPMAAGQASRPSAWRSGPVGDPSTLPGSHRSPKP

| | | |
|---|---|---|
| NOV4q, SNP13382458 of | SEQ ID NO: 63 | 10136 bp |
| CG50377-01, DNA Sequence | ORF Start: ATG at 1 | ORF Stop: TGA at 9313 |
| | SNP Pos: 4429 | SNP Change: A to G |

ATGGCGGGCGCCCCTCCCCCCGCCTTGCTGCTGCCTTGCAGTTTGATCTCAGACTGCTGTGCTAGCAA
TCAGCGACACTCCGTGGGCGTAGGACCCTCCGAGCTAGTCAAGAAGCAAATTGAGTTGAAGTCTCGAG
GTGTGAAGCTGATGCCCAGCAAAGACAACAGCCAGAAGACGTCTGTGTTAACTCAGGTTGGTGTGTCC
CAAGGACATAATATGTGTCCAGACCCTGGCATACCCGAAAGGGGCAAAAGACTAGGCTCGGATTTCAG
GTTAGGATCCAGCGTCCAGTTCACCTGCAACGAGGGCTATGACCTGCAAGGGTCCAAGCGGATCACCT
GTATGAAAGTGAGCGACATGTTTGCGGCCTGGAGCGACCACAGGCCAGTCTGCCGAGCCCGCATGTGT
GATGCCCACCTTCGAGGCCCCTCGGGCATCATCACCTCCCCCAATTTCCCCATTCAGTATGACAACAA
TGCACACTGTGTGTGGATCATCACAGCACTCAACCCCTCCAAGGTGATCAAGCTCGCCTTTGAGGAGT
TTGATTTGGAGAGGGGCTATGACACCCTGACGGTCGGTGATGGTGGTCAGGATGGGGACCAGAAGACA
GTTCTCTACATGTCTCAAAATGCCTGCAGTGACAGCCCTCACACCCCAGGCTCTCGCATCCCAGAGAG
CATGTCTGGGGACATCTGGAGGCAGAAATGGACTGTACTTGAGATCTGTCGTGACATTAGCAGTTCAG
ATGCAAGGTCAGGTTCAGTGAGGAAGTCTCCAAAGACTTCTAATGCTGTGGAACTTGTTGCTCCTGGG
ACAGAGATCGAGCAGGGCAGTTGCGGTGACCCTGGCATACCTGCATATGGCCGGAGGGAAGGCTCCCG
GTTTCACCACGGTGACACACTCAAGTTTGAGTGCCAGCCCGCCTTTGAGCTGGTGGGACAGAAGGCAA
TCACATGCCAAAAGAATAACCAATGGTCGGCTAAGAAGCCAGGCTGCGTGTTCTCCTGCTTCTTCAAC
TTCACCAGCCCGTCTGGGGTTGTCCTGTCTCCCAACTACCCAGAGGACTATGGCAACCACCTCCACTG
TGTCTGGCTCATCCTGGCCAGGCCTGAGAGCCGCATCCACCTGGCCTTCAACGACATTGACGTGGAGC
CTCAGTTTGATTTCCTGGTCATCAAGGATGGGGCCACCGCCGAGGCGCCCGTCCTGGGCACCTTCTCA
GGAAACCAGCTTCCCTCCTCCATCACAAGCAGTGGCCACGTGGCCCGTCTCGAGTTCCAGACTGACCA
CTCCACAGGGAAGAGGGGCTTCAACATCACTTTTACCACCTTCCGACACAACGAGTGCCCGGATCCTG
GCGTTCCAGTAAATGGCAAACGGTTTGGGGACAGCCTCCAGCTGGGCAGCTCCATCTCCTTCCTCTGT
GATGAAGGCTTCCTTGGGACTCAGGGCTCAGAGACCATCACCTGCGTCCTGAAGGAGGGCAGCGTGGT
CTGGAACAGCGCTGTGCTGCGGTGTGAAGCTCCCTGTGGTGGTCACCTGACTTCGCCCAGCGGCACCA
TCCTCTCTCCGGGCTGGCCTGGCTTCTACAAGGATGCCTTGAGCTGTGCCTGGGTGATTGAGGCCCAG
CCAGGCTACCCCATCAAAATCACCTTCGACAGATTCAAAACCGAGGTCAACTATGACACCCTGGAAGT
ACGCGATGGGCGGACTTACTCAGCGCCCTTGATCGGGGTTTACCACGGGACCCAGGTTCCCCAGTTCC
TCATCAGCACCAGCAACTACCTCTACCTCCTCTTCTCTACCGACAAGAGTCACTCGGACATCGGCTTC
CAGCTCCGCTATGAGACTATAACACTGCAGTCAGACCACTGTCTGGATCCAGGAATCCCAGTAAATGG
ACAGCGTCATGGGAATGACTTCTACGTGGGCGCGCTGGTGACCTTCAGCTGTGACTCGGGCTACACAT
TAAGTGACGGGGAGCCTCTGGAGTGTGAGCCCAACTTCCAGTGGAGCCGGGCCCTGCCCAGTTGTGAA
GCTCTCTGTGGTGGCTTCATTCAAGGCTCCAGTGGGACCATCTTGTCGCCAGGGTTCCCTGACTTCTA
CCCCAACAACTTGAACTGCACCTGGATTATCGAAACATCTCATGGCAAGGGTGTGTTCTTCACTTTCC
ACACCTTCCACCTGGAAAGTGGCCATGACTACCTCCTCATCACTGAGAACGGCAGCTTCACCCAGCCC
CTGAGGCAGCTAACTGGATCTCGGCTGCCAGCTCCCATCAGCGCTGGGCTCTATGGCAACTTCACTGC
CCAGGTCCGCTTCATCTCTGATTTCTCCATGTCATATGAAGGATTCAACATCACCTTCTCAGAGTACG
ACTTGGAGCCCTGTGAGGAGCCCGAGGTCCCAGCCTACAGCATCCGGAAGGGCTTGCAGTTTGGCGTG
GGCGACACCTTGACCTTCTCCTGCTTCCCCGGGTACCGTCTGGAGGGCACCGCCCGCATCACGTGCCT
GGGGGGCAGACGGCGCCTGTGGAGCTCGCCTCTGCCAAGGTGTGTTGCTGAGTGTGGGAATTCAGTCA
CAGGCACTCAGGGTACTTTGCTGTCCCCCAACTTTCCTGTGAACTACAATAACAATCATGAATGCATC
TACTCCATCCAGACCCAGCCAGGGAAGGGAATTCAGCTGAAAGCCAGGGCATTCGAACTCTCCGAAGG
AGATGTCCTCAAGGTTTATGATGGCAACAACAACTCCGCCCGTTTGCTGGGAGTTTTTAGCCATTCTG
AGATGATGGGGGTGACTTTGAACAGCACATCCAGCAGTCTGTGGCTTGATTTCATCACTGATGCTGAA
AACACCAGCAAGGGCTTTGAACTGCACTTTTCCAGCTTTGAACTCATCAAATGTGAGGACCCAGGAAC
CCCCAAGTTTGGCTACAAGGTTCATGATGAAGGTCATTTTGCAGGGAGCTCCGTGTCCTTCAGCTGTG
ACCCTGGATACAGCCTGCGGGGTAGTGAGGAGCTGCTGTGTCTGAGTGGAGAGCGCCGGACCTGGGAC
CGGCCTCTGCCCACCTGTGTCGCCGAGTGTGGAGGGACAGTGAGAGGAGAGGTGTCGGGGCAGGTGCT
GTCACCCGGGTATCCAGCTCCCTATGAACACAATCTCAACTGCATCTGGACCATCGAAGCAGAGGCCG
GCTGCACCATTGGGCTACACTTCCTGGTGTTTGACACAGAGGAGGTTCACGACGTGCTGCGCATCTGG
GATGGGCCTGTGGAGAGCGGGGTTCTGCTGAAGGAGCTGAGTGGCCCGGCCCTGCCCAAGGACCTGCA
TAGCACCTTCAACTCGGTCGTCCTGCAGTTCAGCACTGACTTCTTCACCAGCAAGCAGGGCTTTGCCA
TTCAATTTTCAGTGTCCACAGCAACGTCCTGCAATGACCCTGGGATCCCGCAGAATGGGAGTCGGAGT
GGTGACAGTTGGGAAGCCGGCGACTCCACAGTGTTCCAGTGTGACCCTGGCTACGCGCTGCAGGGAAG
TGCAGAGATCAGCTGTGTGAAGATCGAGAACAGGTTCTTCTGGCAGCCCAGCCCGCCAACATGCATCG
CTCCCTGCGGGGGAGACCTGACAGGACCATCTGGAGTCATCCTCTCACCAAATTACCCAGAACCCTAC
CCGCCAGGCAAGGAGTGTGACTGGAAAGTGACCGTCTCACCAGACTACGTCATCGCCCTGGTATTTAA
CATCTTTAACCTGGAGCCTGGCTATGACTTCCTCCATATCTACGACGGACGGGACTCTCTCAGCCCTC
TCATAGGAAGCTTCTATGGCTCCCAGCTCCCAGGCCGCATTGAAAGCAGCAGCAACAGCCTCTTCCTC
GCCTTCCGCAGCGATGCATCTGTGAGCAATGCTGGCTTCGTCATTGACTATACAGAAAACCCGCGGGA
GTCATGTTTTGATCCTGGTTCCATCAAGAACGGCACACGGGTGGGGTCCGACCTGAAGCTGGGCTCCT
CCGTCACCTACTACTGCCACGGGGGCTACGAAGTTGAGGGCACCTCGACCCTGAGCTGCATCCTGGGG
CCTGATGGGAAGCCCGTGTGGAACAATCCCCGGCCAGTCTGCACAGCCCCCTGTGGGGGACAGTATGT
GGGTTCGGACGGAGTGGTCTTGTCCCCCAACTACCCCCAGAACTACACCAGTGGACAGATCTGCTTGT
ATTTTGTTACTGTGCCCAAGGACTATGTGGTGTTTGGCCAGTTCGCCTTCTTTCACACGGCCCTCCCA
GACGTGGTGGAGGTTCACGACGGCCACAGCCAGCACTCGCGGCTCCTCAGCTCCCTCTCGGGCTCCCA
TACAGGAGAATCACTGCCCTTGGCCACCTCCAATCAAGTTCTCATTAAGTTCAGCGCCAAAGGCCTCG
CACCAGCCGGAGGCTTCCACTTTGTCTACCAAGCGGTTCCTCGAACCAGCGCCACGCAGTGCAGCTCT
GTGCCGGAACCCCGCTATGGCAAGAGGCTGGGCAGTGACTTCTCGGTGGGGGCCATCGTCCGCTTCGA
ATGCAACTCCGGCTATGCCCTGCAGGGGTCGCCAGAGATCGAGTGCCTCCCTGTGCCTGGGGCCTTGG
CCCAATGGAATGTCTCAGCGCCCACGTGTGTGGTGCCGTGTGGAGGCAACCTCACAGAGCGCAGGGGC
ACCATCCTGTCCCCTGGCTTCCCAGAGCCGTACCTCAACAGCCTCAACTGTGTGTGGAAGATCGTGGT
CCCCGAAGGCGCTGGCATCCAGATCCAAGTTGTCAGTTTTGTGACAGAGCAGAACTGGGACTCGCTGG
AAGTATTTGATGGTGCAGATAACACTGTAACCATGCTGGGGAGTTTCTCAGGAACAACCGTGCCTGCC
CTTCTGAACAGCACCTCCAACCAGCTCTACCTTCATTTCTACTCAGATATCAGCGTATCTGCAGCTGG
CTTCCACTTGGAGTACAAAACGGTGGGCCTGAGCAGTTGTCCGGAACCTGCTGTGCCCAGTAACGGGG
TGAAGACTGGCGAGCGCTACTTGGTGAATGATGTGGTGTCTTTCCAGTGTGAGCCGGGATATGCCCTC
CAGGGCCACGCCCACATCTCCTGCATGCCCGGAACAGTGCGGCGATGGAACTACCCTCCTCCACTCTG
TATTGCACAGTGTGGGGGAACAGTGGAGGAGATGGAGGGGGTGATCCTGAGCCCCGGCTTCCCAGGCA
ACTACCCCAGTAACATGGACTGCTCCTGGAAAATAGCACTGCCCGTGGGCTTTGGAGCTCACATCCAG
TTCCTGAACTTCTCCACCGAGCCCAACCACGACTACATAGAAATCCGGAATGGCCCCTATGAGACCAG
CCGCATGATGGGAAGATTCAGTGGAAGCGAGCTTCCAAGCTCCCTCCTCTCCACGTCCCACGAGACCA
CCGTGTATTTCCACAGCGACCACTCCCAGAATCGGCCAGGATTCAAGCTGGAGTATCAGGCCTATGAA
CTTCAAGAGTGCCCAGACCCAGAGCCCTTTGCCAATGGCATTGTGAGGGGAGCTGGCTACAACGTGGG
ACAATCAGTGACCTTCGAGTGCCTCCCGGGGTATCAATTGACTGGCCACCCTGTCCTCACGTGTCAAC
ATGGCACCAACCGGAACTGGGACCACCCCCTGCCCAAGTGTGAAGTCCCTTGTGGCGGGAACATCACT
TCTTCCAACGGCACTGTGTACTCCCCGGGGTTCCCTAGCCCGTACTCCAGCTCCCAGGACTGTGTCTG
GCTGATCACCGTGCCCATTGGCCATGGCGTCCGCCTCAACCTCAGCCTGCTGCAGACAGAGCCCTCTG
GAGATTTCATCACCATCTGGGATGGGCCACAGCAAACAGCACCACGGCTCGGCGTCTTCACCCGGAGC
ATGGCCAAGAAAACAGTGCAGAGTTCATCCAACCAGGTCCTGCTCAAGTTCCACCGTGATGCAGCCAC
AGGGGGGATCTTCGCCATAGCTTTCTCCGCTTATCCACTCACCAAATGCCCTCCTCCCACCATCCTCC
CCAACGCCGAAGTCGTCACAGAGAATGAAGAATTCAATATAGGTGACATCGTACGCTACAGATGCCTC
CCTGGCTTTACCTTAGTGGGGAATGAAATTCTGACCTGCAAACTTGGAACCTACCTGCAGTTTGAAGG
ACCACCCCCGATATGTGAAGTGCACTGTCCAACAAATGAGCTTCTGACAGACTCCACAGAGGTGATCC
TGAGCCAGAGCTACCCTGGAAGCTATCCCCAGTTCCAGACCTGCTCTTGGCTGGTGAGAGTGGAGCCC
GACTATAACATCTCCCTCACAGTGGAGTACTTCCTCAGCGAGAAGCAATATGATGAGTTTGAGATTTT
TGATGGTCCATCAGGACAGAGTCCTCTGCTGAAAGCCCTCAGTGGGAATTACTCAGCTCCCCTGATTG
TCACCAGCTCAAGCAACTCTGTGTACCTGCGTTGGTCATCTGATCACGCCTACAATCGGAAGGGCTTC
AAGATCCGCTATTCAGCCCCTTACTGCAGCCTGCCCAGGGCTCCACTCCATGGCTTCATCCTAGGCCA
GACCAGCACCCAGCCCGGGGGCTCCATCCACTTTGGCTGCAACGCCGGCTACCGCCTGGTGGGACACA
GCATGGCCATCTGTACCCGGCACCCCCAGGGCTACCACCTGTGGAGCGAAGCCATCCCTCTCTGTCAA
GCTCTTTCCTGTGGGCTTCCTGAGGCCCCCAAGAATGGAATGGTGTTTGGCAAGGAGTACACAGTGGG
AACCAAGGCCGTGTACAGCTGCAGTGAAGGCTACCACCTCCAGGCAGGCGCTGAGGCCACTGCAGAGT
GTCTGGACACAGGCCTATGGAGCAACCGCAATGTCCCACCACAGTGTGTCCCTGTGACTTGTCCTGAT
GTCAGTAGCATCAGCGTGGAGCATGGCCGATGGAGGCTTATCTTTGAGACACAGTATCAGTTCCAGGC
CCAGCTGATGCTCATCTGTGACCCTGGCTACTACTATACTGGCCAAAGGGTCATCCGCTGTCAGGCCA
ATGGCAAATGGAGCCTCGGGGACTCTACGCCCACCTGCCGAATCATCTCCTGTGGAGAGCTCCCGATT
CCCCCCAATGGCCACCGCATCGGAACACTGTCTGTCTACGGGGCAACAGCCATCTTCTCCTGCAATTC
CGGATACACACTGGTGGGCTCCAGGGTGCGTGAGTGCATGGCCAATGGGCTCTGGAGTGGCTCTGAAG
TCCGCTGCCTTGCTGGACACTGTGGGACTCCTGAGCCCATTGTCAACGGACACATCAATGGGGAGAAC
TACAGCTACCGGGGCAGTGTGGTGTACCAATGCAATGCTGGCTTCCGCCTGATCGGCATGTCTGTGCG
CATCTGCCAGCAGGATCATCACTGGTCGGGCAAGACCCCTTTCTGTGTGCCAATTACCTGTGGACACC
CAGGCAACCCTGTCAACGGCCTCACTCAGGGTAACCAGTTTAACCTCAACGATGTGGTCAAGTTTGTT
TGCAACCCTGGGTATATGGCTGAGGGGGCTGCTAGGTCCCAATGCCTGGCCAGCGGGCAATGGAGTGA
CATGCTGCCCACCTGCAGAATCATCAACTGTACAGATCCTGGACACCAAGAAAATAGTGTTCGTCAGG
TCCACGCCAGCGGCCCGCACAGGTTCAGCTTCGGCACCACTGTGTCTTACCGGTGCAACCACGGCTTC
TACCTCCTGGGCACCCCAGTGCTCAGCTGCCAGGGAGATGGCACATGGGACCGTCCCCGCCCCCAGTG
TCTCTTGGTGTCCTGTGGCCATCCGGGCTCCCCGCCTCACTCCCAGATGTCTGGAGACAGTTATACTG
TGGGAGCAGTGGTGCGGTACAGCTGCATCGGCAAGCGTACTCTGGTGGGAAACAGCACCCGCATGTGT
GGGCTGGATGGACACTGGACTGGCTCCCTCCCTCACTGCTCAGGAACCAGCGTGGGAGTTTGCGGTGA
CCCTGGGATCCCGGCTCATGGCATCCGTTTGGGGGACAGCTTTGATCCAGGCACTGTGATGCGCTTCA
GCTGTGAAGCTGGCCACGTGCTCCGGGGATCGTCAGAGCGCACCTGTCAAGCCAATGGCTCGTGGAGC
GGCTCGCAGCCTGAGTGTGGAGTGATCTCTTGTGGGAACCCTGGGACTCCAAGTAATGCCCGAGTTGT
GTTCAGTGATGGCCTGGTTTTCTCCAGCTCTATCGTCTATGAGTGCCGGGAAGGATACTACGCCACAG
GCCTGCTCAGCCGTCACTGCTCGGTCAATGGTACCTGGACAGGCAGTGACCCTGAGTGCCTCGTCATA
AACTGTGGTGACCCTGGGATTCCAGCCAATGGCCTTCGGCTGGGCAATGACTTCAGGTACAACAAAAC
TGTGACATATCAGTGTGTCCCTGGCTATATGATGGAGTCACATAGAGTATCTGTGCTGAGCTGCACCA
AGGACCGGACATGGAATGGAACCAAGCCCGTCTGCAAAGCTCTCATGTGCAAGCCACCTCCGCTCATC
CCCAATGGGAAGGTGGTGGGGTCTGACTTCATGTGGGGCTCAAGTGTGACTTATGCCTGCCTGGAGGG
GTACCAGCTCTCCCTGCCCGCGGTGTTCACCTGTGAGGGAAATGGGTCCTGGACCGGAGAGCTGCCTC
AGTGTTTCCCTGTGTTCTGCGGGGATCCTGGTGTCCCGTCCCGTGGGAGGAGAGAGGACCGAGGCTTC
TCCTACAGGTCATCTGTCTCCTTCTCCTGCCATCCCCCTCTGGTGCTGGTGGGCTCTCCACGCAGGTT
TTGCCAGTCAGATGGGACATGGAGTGGCACCCAGCCCAGCTGCATAGATCCGACCCTGACCACGTGTG
CGGACCCTGGTGTGCCACAGTTTGGGATACAGAACAATTCTCAGGGCTACCAGGTTGGAAGCACAGTC
CTCTTCCGTTGTCAAAAAGGCTACCTGCTTCAGGGCTCCACCACCAGGACCTGCCTCCCAAACCTGAC
CTGGAGTGGAACCCCACCTGACTGTGTCCCCCACCACTGCAGGCAGCCAGAGACGCCAACGCATGCCA
ACGTCGGGGCCCTGGATTTGCCCTCCATGGGCTACACGCTCATTACTCCTGCCAGGAGGGCTTCTCCC
TCAAGGGTGGCTCCGAGCACCGCACCTGCAAGGCGGATGGCAGCTGGACAGGCAAGCCGCCCATCTGC
CTGGAGGTCCGGCCCAGTGGGAGACCCATCAACACTGCCCGGGAGCCACCGCTCACCCAAGCCTTGAT
TCCTGGGGATGTTTTTGCCAAGAATTCCCTGTGGAAAGGGGCCTATGAATACCAGGGGAAGAAGCAGC
CAGCCATGCTCAGAGTGACTGGCTTCCAAGTTGCCAACAGCAAGGTCAATGCCACCATGATCGACCAC
AGTGGCGTGGAGCTGCACTTGGCTGGAACTTACAAGAAAGAAGATTTTCATCTCCTACTCCAGGTGTA
CCAGATTACAGGGCCTGTGGAGATCTTTATGAATAAGTTCAAAGATGATCACTGGGCTTTAGATGGCC
ATGTCTCGTCAGAGTCCTCCGGAGCCACCTTCATCTACCAAGGCTCTGTCAAGGGCCAAGGCTTTGGG
CAGTTCGGCTTTCAAAGACTGGACCTCAGGCTGCTGGAGTCAGACCCCGAGTCCATTGGCCGCCACTT
TGCTTCCAACAGCAGCTCAGTGGCAGCCGCGATCCTGGTGCCTTTCATCGCCCTCATTATTGCGGGCT
TCGTGCTCTATCTCTACAAGCACAGGAGAAGACCCAAAGTTCCTTTCAATGGCTATGCTGGCCACGAG
AACACCAATGTTCGGGCCACATTTGAGAACCCAATGTACGACCGCAACATCCAGCCCACAGACATCAT
GGCCAGCGAGGCGGAGTTCACAGTCAGCACAGTGTGCACAGCAGTATAGCCACCCGGCCTGGCCGCTT
TTTTTGCTAGGTTGAACTGGTACTCCAGCAGCCGCCGAAGCTGGACTGTACTGCTGCCATCTCAGCTC
ACTGCAACCTCCCTGCCTGATTCCCCTGCCTCAGCCTGCCGAGTGCCTGCGATTGCAGGCGCGCACCG
CCAC

| | | | |
|---|---|---|---|
| NOV4q, SNP1 3382458 of | SEQ ID NO: 64 | 3104 aa | MW at 336647.2kD |
| CG50377-01, | SNP Pos: 1477 | SNP Change: Arg to Gly | |
| Protein Sequence | | | |

MAGAPPPALLLPCSLISDCCASNQRHSVGVGPSELVKKQIELKSRGVKLMPSKDNSQKTSVLTQVGVS
QGHNNCPDPGIPERGKRLGSDFRLGSSVQFTCNEGYDLQGSKRITCMKVSDMFAAWSDHRPVCRARMC
DAHLRGPSGIITSPNFPIQYDNNAHCVWIITALNPSKVIKLAFEEFDLERGYDTLTVGDGGQDGDQKT
VLYMSQNACSDSPHTPGSRIPESMSGDIWRQKWTVLEICRDISSSDARSGSVRKSPKTSNAVELVAPG
TEIEQGSCGDPGIPAYGRREGSRFHHGDTLKFECQPAFELVGQKAITCQKNNQWSAKKPGCVFSCFFN
FTSPSGVVLSPNYPEDYGNHLHCVWLILARPESRIHLAFNDIDVEPQFDFLVIKDGATAEAPVLGTFS
GNQLPSSITSSGHVARLEFQTDHSTGKRGFNITFTTFRHNECPDPGVPVNGKRFGDSLQLGSSISFLC
DEGFLGTQGSETITCVLKEGSVVWNSAVLRCEAPCGGHLTSPSGTILSPGWPGFYKDALSCAWVIEAQ
PGYPIKITFDRFKTEVNYDTLEVRDGRTYSAPLIGVYHGTQVPQFLISTSNYLYLLFSTDKSHSDIGF
QLRYETITLQSDHCLDPGIPVNGQRHGNDFYVGALVTFSCDSGYTLSDGEPLECEPNFQWSRALPSCE
ALCGGFIQGSSGTILSPGFPDFYPNNLNCTWIIETSHGKGVFFTFHTFHLESGHDYLLITENGSFTQP
LRQLTGSRLPAPISAGLYGNFTAQVRPISDFSMSYEGFNITFSEYDLEPCEEPEVPAYSIRKGLQFGV
GDTLTFSCFPGYRLEGTARITCLGGRRRLWSSPLPRCVAECGNSVTGTQGTLLSPNFPVNYNNNHECI
YSIQTQPGKGIQLKARAFELSEGDVLKVYDGNNNSARLLGVFSHSEMMGVTLNSTSSSLWLDFITDAE
NTSKGFELHFSSFELIKCEDPGTPKFGYKVHDEGHFAGSSVSFSCDPGYSLRGSEELLCLSGERRTWD
RPLPTCVAECGGTVRGEVSGQVLSPGYPAPYEHNLNCIWTIEAEAGCTIGLHFLVFDTEEVHDVLRIW
DGPVESGVLLKELSGPALPKDLHSTFNSVVLQFSTDFFTSKQGFAIQFSVSTATSCNDPGIPQNGSRS
GDSWEAGDSTVFQCDPGYALQGSAEISCVKIENRFFWQPSPPTCIAPCGGDLTGPSGVILSPNYPEPY
PPGKECDWKVTVSPDYVIALVFNIFNLEPGYDFLHIYDGRDSLSPLIGSFYGSQLPGRIESSSNSLFL
AFRSDASVSNAGFVIDYTENPRESCFDPGSIKNGTRVGSDLKLGSSVTYYCHGGYEVEGTSTLSCILG
PDGKPVWNNPRPVCTAPCGGQYVGSDGVVLSPNYPQNYTSGQICLYFVTVPKDYVVFGQFAFFHTALN
DVVEVHDGHSQHSRLLSSLSGSHTGESLPLATSNQVLIKFSAKGLAPAGGFHFVYQAVPRTSATQCSS
VPEPRYGKRLGSDFSVGAIVRFECNSGYALQGSPEIECLPVPGALAQWNVSAPTCVVPCGGNLTERRG
TILSPGFPEPYLNSLNCVWKIVVPEGAGIQIQVVSFVTEQNWDSLEVFDGADNTVTMLGSFSGTTVPA
LLNSTSNQLYLHFYSDISVSAAGFHLEYKTVGLSSCPEPAVPSNGVKTGERYLVNDVVSFQCEPGYAL
QGHAHISCMPGTVRRWNYPPPLCIAQCGGTVEEMEGVILSPGFPGNYPSNMDCSWKIALPVGFGAHIQ
FLNFSTEPNHDYIEIRNGPYETSRMMGRFSGSELPSSLLSTSHETTVYFHSDHSQNRPGFKLEYQAYE
LQECPDPEPFANGIVRGAGYNVGQSVTFECLPGYQLTGHPVLTCQHGTNRNWDHPLPKCEVPCGGNIT
SSNGTVYSPGFPSPYSSSQDCVWLITVPIGHGVRLNLSLLQTEPSGDFITIWDGPQQTAPRLGVFTRS
MAKKTVQSSSNQVLLKFHRDAATGGIFAIAFSAYPLTKCPPPTILPNAEVVTENEEFNIGDIVRYRCL
PGFTLVGNEILTCKLGTYLQFEGPPPICEVHCPTNELLTDSTGVILSQSYPGSYPQFQTCSWLVRVEP
DYNISLTVEYFLSEKQYDEFEIFDGPSGQSPLLKALSGNYSAPLIVTSSSNSVYLRWSSDHAYNRKGF
KIRYSAPYCSLPRAPLHGFILGQTSTQPGGSIHFGCNAGYRLVGHSMAICTRHPQGYHLWSEAIPLCQ
ALSCGLPEAPKNGMVFGKEYTVGTKAVYSCSEGYHLQAGAEATAECLDTGLWSNRNVPPQCVPVTCPD
VSSISVEHGRWRLIFETQYQFQAQLMLICDPGYYYTGQRVIRCQANGKWSLGDSTPTCRIISCGELPI
PPNGHRIGTLSVYGATAIFSCNSGYTLVGSRVRECMANGLWSGSEVRCLAGHCGTPEPIVNGHINGEN
YSYRGSVVYQCNAGFRLIGMSVRICQQDHHWSGKTPFCVPITCGHPGNPVNGLTQGNQFNLNDVVKFV
CNPGYMAEGAARSQCLASGQWSDMLPTCRIINCTDPGHQENSVRQVHASGPHRFSFGTTVSYRCNHGF
YLLGTPVLSCQGDGTWDRPRPQCLLVSCGHPGSPPHSQMSGDSYTVGAVVRYSCIGKRTLVGNSTPMC
GLDGHWTGSLPHCSGTSVGVCGDPGIPAHGIRLGDSFDPGTVMRFSCEAGHVLRGSSERTCQANGSWS
GSQPECGVISCGNPGTPSNARVVFSDGLVFSSSIVYECREGYYATGLLSRHCSVNGTWTGSDPECLVI
NCGDPGIPANGLRLGNDFRYNKTVTYQCVPGYMMESHRVSVLSCTKDRTWNGTKPVCKALMCKPPPLI
PNGKVVGSDFMWGSSVTYACLEGYQLSLPAVFTCEGNGSWTGELPQCFPVFCGDPGVPSRGRREDRGF
SYRSSVSFSCHPPLVLVGSPRRFCQSDGTWSGTQPSCIDPTLTTCADPGVPQFGIQNNSQGYQVGSTV
LFRCQKGYLLQGSTTRTCLPNLTWSGTPPDCVPHHCRQPETPTHANVGALDLPSMGYTLITPARRASP
SRVAPSTAPARRMAAGQASRPSAWRSGPVGDPSTLPGSHRSPKP

A ClustalW comparison of the above protein sequences yields the following sequence alignment shown in Table 4B.

TABLE 4B

Comparison of the NOV4 protein sequences.

| | |
|---|---|
| NOV4a | ------------------------------------------------------------ |
| NOV4b | MAGAPPPALLLPCSLISDCCASNQRHSVGVGPSELVKKQIELKSRGVKLMPSKDNSQKTS |
| NOV4c | ------------------------------------------------------------ |
| NOV4d | ------------------------------------------------------------ |
| NOV4e | ------------------------------------------------------------ |
| NOV4f | ------------------------------------------------------------ |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| Nov4l | ------------------------------------------------------------ |
| NOV4m | ------------------------------------------------------------ |
| NOV4n | ------------------------------------------------------------ |
| NOV4o | ------------------------------------------------------------ |
| NOV4a | ------------------------------------------------------------ |
| NOV4b | VLTQVGVSQGHNMCPDPGIPERGKRLGSDFRLGSSVQFTCNEGYDLQGSKRITCMKVSDM |
| NOV4c | ------------------------------------------------------------ |
| NOV4d | ------------------------------------------------------------ |
| NOV4e | ------------------------------------------------------------ |
| NOV4f | ------------------------------------------------------------ |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| Nov4l | ------------------------------------------------------------ |
| NOV4m | ------------------------------------------------------------ |
| NOV4n | ------------------------------------------------------------ |
| NOV4o | ------------------------------------------------------------ |
| NOV4a | ------------------------------------------------------------ |
| NOV4b | FAAWSDHRPVCRARMCDAHLRGPSGIITSPNFPIQYDNNAHCVWIITALNPSKVIKLAFE |
| NOV4c | ------------------------------------------------------------ |
| NOV4d | ------------------------------------------------------------ |
| NOV4e | ------------------------------------------------------------ |
| NOV4f | ------------------------------------------------------------ |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| Nov4l | ------------------------------------------------------------ |
| NOV4m | ------------------------------------------------------------ |
| NOV4n | ------------------------------------------------------------ |
| NOV4o | ------------------------------------------------------------ |
| NOV4a | ------------------------------------------------------------ |
| NOV4b | EFDLERGYDTLTVGDGGQDGDQKTVLYMSQNACSDSPHTPGSRIPESMSGDIWRQKWTVL |
| NOV4c | ------------------------------------------------------------ |
| NOV4d | ------------------------------------------------------------ |
| NOV4e | ------------------------------------------------------------ |
| NOV4f | ------------------------------------------------------------ |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | ------------------------------------------------------------ |
| NOV4b | EICRDISSSDARSGSVRKSPKTSNAVELVAPGTEIEQGSCGDPGIPAYGRREGSRFHHGD |
| NOV4c | ------------------------------------------------------------ |
| NOV4d | ------------------------------------------------------------ |
| NOV4e | ------------------------------------------------------------ |
| NOV4f | ------------------------------------------------------------ |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| Nov4l | ------------------------------------------------------------ |
| NOV4m | ------------------------------------------------------------ |
| NOV4n | ------------------------------------------------------------ |
| NOV4o | ------------------------------------------------------------ |
| NOV4a | -------------------------------GQNCTFQLHGPNGTVESPGFPYGYPNYAN |
| NOV4b | TLKFECQPAFELVGQKAITCQKNNQWSAKKPGCVFSCFFNFTSPSGVVLSPNYPEDYGNH |
| NOV4c | ------------------------------------------------------------ |
| NOV4d | ------------------------------------------------------------ |
| NOV4e | -------------------------------GQNCTFQLHGPNGTVESPGFPYGYPNYAN |
| NOV4f | ------------------------------------------------------------ |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | CTWTITAEEQHRIQLVFQSFALEEDFDVLSVFDGPPQPENLRTRLTGFQLPATIVSAATT |
| NOV4b | LHCVWLILARPESRIHLAFNDIDVEPQFDFLVIKDGATAEAPVLGTFSGNQLPSSITSSG |
| NOV4c | ------------------------------------------------------------ |
| NOV4d | ------------------------------------------------------------ |
| NOV4e | CTWTITAEEQHRIQLVFQSFALEEDFDVLSVFDGPPQPENLRTRLTGFQLPATIVSAATT |
| NOV4f | ------------------------------------------------------------ |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| Nov4l | ------------------------------------------------------------ |
| NOV4m | ------------------------------------------------------------ |
| NOV4n | ------------------------------------------------------------ |
| NOV4o | ------------------------------------------------------------ |
| NOV4a | LSLPLISDYAVSAQGFHATYEVLPSHTCGNPGRLPNGIQQGSTFNLGDKVRYSCNLGFFL |
| NOV4b | NVARLEFQTDHSTGKRGFNITFTTFRHNECPDPGVPVNGKRFGDSLQLGSSISFLCDEGF |
| NOV4c | ------------------------------------------------------------ |
| NOV4d | ------------------------------------------------------------ |
| NOV4e | LSLRLISDYAVSAQGFHATYEVLPSHTCGNPGRLPNGIQQGSTFNLGDKVRYSCNLGFFL |
| NOV4f | ------------------------------------------------------------ |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| Nov4l | ------------------------------------------------------------ |
| NOV4m | ------------------------------------------------------------ |
| NOV4n | ------------------------------------------------------------ |
| NOV4o | ------------------------------------------------------------ |
| NOV4a | EGHAVLTCHAGSENSATWDFPLPSCRADDACGGTLRGQSGIISSPHFPSEYHNNADCTWT |
| NOV4b | LGTQGSETITCVLKEGSVVWNSAVLRCEAPCGGHLTSPSGTILSPGWPGFYKDALSCAWV |
| NOV4c | ------------------------------------------------------------ |
| NOV4d | ------------------------------------------------------------ |
| NOV4e | EGHAVLTCHAGSENSATWDFPLPSCRADDACGGTLRGQSGIISSPHFPSEYHNNADCTWT |
| NOV4f | ------------------------------------------------------------ |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| Nov4l | ------------------------------------------------------------ |
| NOV4m | ------------------------------------------------------------ |
| NOV4n | ------------------------------------------------------------ |
| NOV4o | ------------------------------------------------------------ |
| NOV4a | ILAELGDTIALVFIDFQLEDGYDFLEVTGTEGSSLWFTGASLPAPVISSKNWLRLHFTSD |
| NOV4b | IEAQPGYPIKITFDRFKTEVNYDTLEVRDGRTYSAPLIGVYHGTQVPQFLISTSNYLYLL |
| NOV4c | --------------------------------------MAGAPPPALLLPCSLISDCCAS |
| NOV4d | --------------------------------------MAGAPPPALLLPCSLISDCCAS |
| NOV4e | ILAELGDTIALVFIDFQLEDGYDFLEVTGTEGSSLWFTGASLPAPVISSKNWLRLHFTSD |
| NOV4f | ------------------------------------------------------------ |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | GNHRQRGFSAQYQVKKQIELKSRGVKLMPSKDNSQKTSVVTQVGVSQGHNMCPDPGIPER |
| NOV4b | FSTDKSHSDIGFQLRYETITLQSDHCLDPGIPVNGQRHGNDFYVGALVTFSCDSGYTLSD |
| NOV4c | NQRHSVGVGPSELVKKQIELKSRGVKLMPSKDNSQKTSVLTQVGVSQGHNMCPDPGIPER |
| NOV4d | NQRHSVGVGPSELVKKQIELKSRGVKLMPSKDNSQKTSVLTQVGVSQGHNMCPDPGIPER |
| NOV4e | GNHRQRGFSAQYQVKKQIELKSRGVKLMPSKDNSQKTSVVTQVGVSQGHNMCPDPGIPER |
| NOV4f | ---------------------------MPSKDNSQKTSVVTQVGVSQGHNMCPDPGIPER |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| Nov4l | ------------------------------------------------------------ |
| NOV4m | ------------------------------------------------------------ |
| NOV4n | ------------------------------------------------------------ |
| NOV4o | ------------------------------------------------------------ |
| NOV4a | GKRLGSDFRLGSSVQFTCNEGYDLQGSKRITCMKVSDMFAAWSDHRPVCRAPMCDAHLRC |
| NOV4b | GEPLECEPNFQWSRALPSCEALCGGFIQGSSGTILSPGFPDFYPNNLNCTWIIETSHGKG |
| NOV4c | GKRLGSDFRLGSSVQFTCNEGYDLQGSKRITCMKVSDMFAAWSDHRPVCRARMCDAHLRG |
| NOV4d | GKRLGSDFRLGSSVQFTCNEGYDLQGSKRITCMKVSDMFAAWSDHRPVCRARMCDAHLRG |
| NOV4e | GKRLGSDFRLGSSVQFTCNEGYDLQGSKRITCMKVSDMFAAWSDHRPVCRAPMCDAHLRG |
| N0V4f | GKRLGSDFRLGSSVQFTCNEGYDLQGSKRITCMKVSDMFAAWSDHRPVCRAENCDAHLRG |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| Nov4l | ------------------------------------------------------------ |
| NOV4m | ------------------------------------------------------------ |
| NOV4n | ------------------------------------------------------------ |
| NOV4o | ------------------------------------------------------------ |
| NOV4a | PSGIITSPNFPIQYDNNAHCVWIITALNPSKVIKLAFEEFDLERGYDTLTVGDGGQDGDQ |
| NOV4b | VFFTFHTFHLESGHDYLLITENGSFTQPLRQLTGSRLPAPISAGLYGNFTAQVRFISDFS |
| NOV4c | PSGIITSPNFPIQYDNNAHCVWIITALNPSKVIKLAFEEFDLERGYDTLTVGDGGQDGDQ |
| NOV4d | PSGIITSPNFPIQYDNNAHCVWIITALNPSKVIKLAFEEFDLERGYDTLTVGDGGQDGDQ |
| NOV4e | PSGIITSPNFPIQYDNNAHCVWIITALNPSKVIKLAFEEFDLERGYDTLTVGDGGQDGDQ |
| NOV4f | PSGIITSPNFPIQYDNNAHCVWIITALNPSKVIKLAFEEFDLERGYDTLTVGDGGQDGDQ |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| Nov4l | ------------------------------------------------------------ |
| NOV4m | ------------------------------------------------------------ |
| NOV4n | ------------------------------------------------------------ |
| NOV4o | ------------------------------------------------------------ |
| NOV4a | KTVLYMLTGTSVPDLIVSTNHQMWLLFQTDG---------------------SG------ |
| NOV4b | MSYEGFNITFSEYDLEPCEEPEVPAYSIRKGLQFGVGDTLTFSCFPGYRLEGTARITCLG |
| NOV4c | KTVLYMSQNACSDSPHTPGSRIPESMSGDIWRQKWTVLEICRDISSSDARSGSVRKSPKT |
| NOV4d | KTVLYMSQNACSDSPHTPGSRIPESMSGDIWRQKWTVLEICRDISSSDARSGSVRKSPKT |
| NOV4e | KTVLYMLTGTSVPDLIVSTNHQMWLLFQTDG----------------------------S |
| NOV4f | KTVLYMLTGTSVPDLIVSTNHQMWLLFQTDG----------------------S------ |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | -SSLGFKASYEEIEQGSCGDPGIPAYGRREGSRFRHGDTLKFECQPAFELVGQKAITCQK |
| NOV4b | GRRPLWSSPLPRCVAECGNSVTGTQGTLLSPNFPVNYNNNHECIYSIQTQPGKGIQLKAR |
| NOV4c | SNAVELVAPGTEIEQGSCGDPGIPAYGRREGSRFHHGDTLKFECQPAFELVGQKAITCQK |
| NOV4d | SNAVELVAPGTEIEQGSCGDPGIPAYGRREGSRFHHGDTLKFECQPAFELVGQKAITCQK |
| NOV4e | GSSLGFKASYEEIEQGSCGDPGIPAYGRREGSRFRHGDTLKFECQPAFELVGQKAITCQK |
| NOV4f | GSSLGFKASYEEIEQGSCGDPGIPAYGRREGSRFRHGDTLKFECQPAFELVGQKAITCQK |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | NNQWSAKKPGCVCSCFFNFTSPSGVVLSPNYPEDYGNHLHCVWLILARPESRIHLAFNDI |
| NOV4b | AFELSEGDVLKVYDGNNNSARLLGVFSHSEMMGVTLNSTSSSLWLDFITDAENTSKGFEL |
| NOV4c | NNQWSAKKPGCVFSCFFNFTSPSGVVLSPNYPEDYGNHLHCVWLILARPESRIHLAFNDI |
| NOV4d | NNQWSAKKPGCVFSCFFNFTSPSGVVLSPNYPEDYGNHLHCVWLILARPESRIHLAFNDI |
| NOV4e | NNQWSAKKPGCVCSCFFNFTSPSGVVLSPNYPEDYGNHLHCVWLILARPESRIHLAFNDI |
| NOV4f | NNQWSAKKPGCVCSCFFNFTSPSGVVLSPNYPEDYGNHLHCVWLILARPESRIHLAFNDI |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | DVEPQFDFLVIKDGATAEAPVLGTFSGNQLPSSITSSGHVARLEFQTDHSTGKRGFNITF |
| NOV4b | HFSSFELIKCEDPGTPKFGYKVHDEGHFAGSSVSFSCDPGYSLRGSEELLCLSGERRTWD |
| NOV4c | DVEPQFDFLVIKDGATAEAPVLGTFSGNQLPSSITSSGHVARLEFQTDHSTGKRGFNITF |
| NOV4d | DVEPQFDFLVIKDGATAEAPVLGTFSGNQLPSSITSSGHVARLEFQTDHSTGKRGFNITF |
| NOV4e | DVEPQFDFLVIKDGATAEAPVLGTFSGNQLPSSITSSGHvARLEFQTDHSTGKRGFNITF |
| NOV4f | DVEPQFDFLVIKDGATAEAPVLGTFSGNQLPSSITSSGHVARLEFQTDHSTGKRGFNITF |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | TSESSNECPDPGVPVNGKRFGDSLQLGSSISFLCDEGFLGTQGSETITCVLKEGSVVWNS |
| NOV4b | RPLPTCVAECGGTVRGEVSGQVLSPGYPAPYEHNLNCIWTIEAEAGCTIGLHFLVFDTEE |
| NOV4c | TTFRHNECPDPGVPVNGKRFGDSLQLGSSISFLCDEGFLGTQGSETITCVLKEGSVVWNS |
| NOV4d | TTFRHNECPDPGVPVNGKRFGDSLQLGSSISFLCDEGFLGTQGSETITCVLKEGSVVWNS |
| NOV4e | TSESSNECPDPGVPVNGKRFGDSLQLGSSISFLCDEGFLGTQGSETITCVLKEGSVVWNS |
| NOV4f | TSESSNECPDPGVPVNGKRFGDSLQLGSSISFLCDEGFLGTQGSETITCVLKEGSVVWNS |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| Nov4l | ------------------------------------------------------------ |
| NOV4m | ------------------------------------------------------------ |
| NOV4n | ------------------------------------------------------------ |
| NOV4o | ------------------------------------------------------------ |
| NOV4a | AVLRCEAPCGGHLTSPSGTILSPGWPGFYKDALSCAWVIEAQPGYPIKITFDRFKTEVNY |
| NOV4b | VHDVLRIWDGPVESGVLLKELSGPALPKDLHSTFNSVVLQFSTDFFTSKQGFAIQFSVST |
| NOV4c | AVLRCEAPCGGHLTSPSGTILSPGWPGFYKDALSCAWVIEAQPGYPIKITFDRFKTEVNY |
| NOV4d | AVLRCEAPCGGHLTSPSGTILSPGWPGFYKDALSCAWVIEAQPGYPIKITFDRFKTEVNY |
| NOV4e | AVLRCEAPCGGHLTSPSGTILSPGWPGFYKDALSCAWVIEAQPGYPIKITFDRFKTEVNY |
| NOV4f | AVLRCEAPCGGHLTSPSGTILSPGWPGFYKDALSCAWVIEAQPGYPIKITFDRFKTEVNY |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | DTLEVRDGRTYSAPLIGVYHGTQVPQFLISTSNYLYLLFSTDKSHSDIGFQLRYEAITLQ |
| NOV4b | ATSCNDPGIPQNGSRSGDSWEAGDSTVFQCDPGYALQGSAEISCVKIENRFFWQPSPPTC |
| NOV4c | DTLEVRDGRTYSAPLIGVYHGTQVPQFLISTSNYLYLLFSTDKSHSDIGFQLRYETITLQ |
| NOV4d | DTLEVRDGRTYSAPLIGVYHGTQVPQFLISTSNYLYLLFSTDKSHSDIGFQLRYETITLQ |
| NOV4e | DTLEVRDGRTYSAPLIGVYHGTQVPQFLISTSNYLYLLFSTDKSHSDIGFQLRYEAITLQ |
| NOV4f | DTLEVRDGRTYSAPLIGVYHGTQVPQFLISTSNYLYLLFSTDKSHSDIGFQLRYEAITLQ |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | SDHCLDPGIPVNGQRHGNDFYVGALVTFSCDSGYTLSDGEPLECEPNFQWSRALPSCEAL |
| NOV4b | IAPCGGDLTGPSGVILSPNYPEPYPPGKECDWKVTVSPDYVIALVFNIFNLEPGYDFLHI |
| NOV4c | SDHCLDPGIPVNGQRHGNDFYVGALVTFSCDSGYTLSDGEPLECEPNFQWSRALPSCEAL |
| NOV4d | SDHCLDPGIPVNGQRHGNDFYVGALVTFSCDSGYTLSDGEPLECEPNFQWSRALPSCEAL |
| NOV4e | SDHCLDPGIPVNGQRHGNDFYVGALVTFSCDSGYTLSDGEPLECEPNFQWSRALPSCEAL |
| NOV4f | SDHCLDPGIPVNGQRHGNDFYVGALVTFSCDSGYTLSDGEPLECEPNFQWSRALPSCEAL |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | CGGFIQGSSGTILSPGFPDFYPNNLNCTWIIETSHGKGVFFTFHTFHLESGHDYLLITEN |
| NOV4b | YDGRDSLSPLIGSFYGSQLPGRIESSSNSLFLAFRSDASVSNAGFVIDYTENPRESCFDP |
| NOV4c | CGGFIQGSSGTILSPGFPDFYPNNLNCTWIIETSHGKGVFFTFHTFHLESGHDYLLITEN |
| NOV4d | CGGFIQGSSGTILSPGFPDFYPNNLNCTWIIETSHGKGVFFTFHTFHLESGHDYLLITEN |
| NOV4e | CGGFIQGSSGTILSPGFPDFYPNNLNCTWITETSHGKGVFFTFHTFHLESGHDYLLITEN |
| NOV4f | CGGFIQGSSGTILSPGFPDFYPNNLNCTWIIETSHGKGVFFTFHTFHLESGHDYLLITEN |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | GSFTQPLRQLTGSRLPAPISAGLYGNFTAQVRFISDFSMSYEGFNITFSEYDLEPCEEPE |
| NOV4b | GSIKNGTRVGSDLKLGSSVTYYCHGGYEVEGTSTLSCILGPDGKPVWNNPRPVCTAPCGG |
| NOV4c | GSFTQPLRQLTGSRLPAPISAGLYGNFTAQVRFISDFSMSYEGFNITFSEYDLEPCEEPE |
| NOV4d | GSFTQPLRQLTGSRLPAPISAGLYGNFTAQVRFISDFSMSYEGFNITFSEYDLEPCEEPE |
| NOV4e | GSFTQPLRQLTGSRLPAPISAGLYGNFTAQVRFISDFSMSYEGFNITFSEYDLEPCEEPE |
| NOV4f | GSFTQPLRQLTGSRLPAPISAGLYGNFTAQVRFISDFSMSYEGFNITFSEYDLEPCEEPE |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | VPAYSIRKGLQFGVGDTLTFSCFPGYRLEGTARITCLGGPRRLWSSPLPRCVAECGNSVT |
| NOV4b | QYVGSDGVVLSPNYPQNYTSGQICLYFVTVPKDYVVFGQFAFFHTALNDVVEVHDGHSQH |
| NOV4c | VPAYSIRKGLQFGVGDTLTFSCFPGYRLEGTARITCLGGRRRLWSSPLPRCVAECGNSVT |
| NOV4d | VPAYSIRKGLQFGVGDTLTFSCFPGYRLEGTARITCLGGRRRLWSSPLPRCVAECGNSVT |
| NOV4e | VPAYSIRKGLQFGVGDTLTFSCFPGYRLEGTARITCLGGRRRLWSSPLPRCVAECGNSVT |
| NOV4f | VPAYSIRKGLQFGVGDTLTFSCFPGYRLEGTARITCLGGRRRLWSSPLPRCVAECGNSVT |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | GTQGTLLSPNFPVNYNNNHECIYSIQTQPGKGIQLKARAFELSEGDVLKVYDGNNNSARL |
| NOV4b | SRLLSSLSGSHTGESLPLATSNQVLIKFSAKGLAPARGFHFVYQAVPRTSATQCSSVPEP |
| NOV4c | GTQGTLLSPNFPVNYNNNHECIYSIQTQPGKGIQLKARAFELSEGDVLKVYDGNNNSARL |
| NOV4d | GTQGTLLSPNFPVNYNNNHECIYSIQTQPGKGIQLKARAFELSEGDVLKVYDGNNNSARL |
| NOV4e | GTQGTLLSPNFPVNYNNNHECIYSIQTQPGKGIQLKARAFELSEGDVLKVYDGNNNSARL |
| NOV4f | GTQGTLLSPNFPVNYNNNHECIYSIQTQPGKGIQLKARAFELSEGDVLKVYDGNNNSARL |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| Nov4l | ------------------------------------------------------------ |
| NOV4m | ------------------------------------------------------------ |
| NOV4n | ------------------------------------------------------------ |
| NOV4o | ------------------------------------------------------------ |
| NOV4a | LGVFSHSEMMGVTLNSTSSSLWLDFITDAENTSKGFELHFSSFELIKCEDPGTPKFGYKV |
| NOV4b | RYGKRLGSDFSVGATVRFECNSGYALQGSPEIECLPVPGALAQWNVSAPTCVVPCGGNLT |
| NOV4c | LGVFSHSEMMGVTLNSTSSSLWLDFITDAENTSKGFELHFSSFELIKCEDPGTPKFGYKV |
| NOV4d | LGVFSHSEMNGVTLNSTSSSLWLDFITDAENTSKGFELHFSSFELIKCEDPGTPKFGYKV |
| NOV4e | LGVFSHSEMMGVTLNSTSSSLWLDFITDAENTSKGFELHFSSFELIKCEDPGTPKFGYKV |
| NOV4f | LGVFSHSEMMGVTLNSTSSSLWLDFITDAENTSKGFELHFSSFELIKCEDPGTPKFGYKV |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | HDEGHFAGSSVSFSCDPGYSLRGSEELLCLSGERRTWDRPLPTCVAECGGTVRGEVSGQV |
| NOV4b | ERRGTILSPGFPEPYLNSLNCVWKIVVPEGAGIQIQVVSFVTEQNWDSLEVFDGADNTVT |
| NOV4c | HDEGHFAGSSVSFSCDPGYSLRGSEELLCLSGERRTWDRPLPTCVAECGGTVRGEVSGQV |
| NOV4d | HDEGHFAGSSVSFSCDPGYSLRGSEELLCLSGERRTWDRPLPTCVAECGGTVRGEVSGQV |
| NOV4e | HDEGHFAGSSVSFSCDPGYSLRGSEELLCLSGERRTWDRPLPTCVAECGGTVRGEVSGQV |
| NOV4f | HDEGHFAGSSVSFSCDPGYSLRGSEELLCLSGERRTWDRPLPTCVAECGGTVRGEVSGQV |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| Nov4l | ------------------------------------------------------------ |
| NOV4m | ------------------------------------------------------------ |
| NOV4n | ------------------------------------------------------------ |
| NOV4o | ------------------------------------------------------------ |
| NOV4a | LSPGYPAPYEHNLNCIWTIEAEAGCTIGLHFLVFDTEEVHDVLRIWDGPVESGVLLKELS |
| NOV4b | MLGSFSGTTVPALLNSTSNQLYLHFYSDISVSAAGFHLEYKTVGLSSCPEPAVPSNGVKT |
| NOV4c | LSPGYPAPYEHNLNCIWTIEAEAGCTIGLHFLVFDTEEVHDVLRIWDGPVESGVLLKELS |
| NOV4d | LSPGYPAPYEHNLNCIWTIEAEAGCTIGLHFLVFDTEEVHDVLRIWDGPVESGVLLKELS |
| NOV4e | LSPGYPAPYEHNLNCIWTIEAEAGCTIGLHFLVFDTEEVHDVLRIWDGPVESGVLLKELS |
| NOV4f | LSPGYPAPYEHNLNCIWTIEAEAGCTIGLHFLVFDTEEVHDVLRIWDGPVESGVLLKELS |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | GPALPKDLHSTFNSVVLQFSTDFFTSKQGFAIQFSGSTATSCNDPGIPQNGSRSGDSWEA |
| NOV4b | GERYLVNDVVSFQCEPGYALQGHAHISCMPGTVRRWNYPPPLCIAQCGGTVEEMEGVILS |
| NOV4c | GPALPKDLHSTFNSVVLQFSTDFFTSKQGFAIQFSVSTATSCNDPGIPQNGSRSGDSWEA |
| N0V4d | GPALPKDLHSTFNSVVLQFSTDFFTSKQGFAIQFSVSTATSCNDPGIPQNGSRSGDSWEA |
| NOV4e | GPALPKDLHSTFNSVVLQFSTDFFTSKQGFAIQFSGSTATSCNDPGIPQNGSRSGDSWEA |
| NOV4f | GPALPKDLHSTFNSVVLQFSTDFFTSKQGFAIQFSGSTATSCNDPGIPQNGSRSGDSWEA |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| Nov4l | ------------------------------------------------------------ |
| NOV4m | ------------------------------------------------------------ |
| NOV4n | ------------------------------------------------------------ |
| NOV4o | ------------------------------------------------------------ |
| NOV4a | GDSTVFQCDPGYALQGSAEISCVKIENRFFWQPSPPTCIAPCGGDLTGPSGVILSPNYPE |
| NOV4b | PGFPGNYPSNMDCSWKIALPVGFGAHIQFLNFSTEPNHDYIEIRNGPYETSRMMGRFSGS |
| NOV4c | GDSTVFQCDPGYALQGSAEISCVKIENRFFWQPSPPTCIAPCGGDLTGPSGVILSPNYPE |
| NOV4d | GDSTVFQCDPGYALQGSAEISCVKIENRFFWQPSPPTCIAPCGGDLTGPSGVILSPNYPE |
| NOV4e | GDSTVFQCDPGYALQGSAEISCVKIENRFFWQPSPPTCIAPCGGDLTGPSGVILSPNYPE |
| NOV4f | GDSTVFQCDPGYALQGSAEISCVKIENRFFWQPSPPTCIAPCGGDLTGPSGVILSPNYPE |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | PYPPGKECDWKVTVSPDYVIALVLFPSFNLEPGYDFLHIYDGRDSLSPLIGSFYGSQLPG |
| NOV4b | ELPSSLLSTSHETTVYFHSDHSQNRPGFKLEYQAYELQECPDPEPFANGIVRGAGYNVGQ |
| NOV4c | PYPPGKECDWKVTVSPDYVIALVFN-IFNLEPGYDFLHIYDGRDSLSPLIGSFYGSQLPG |
| NOV4d | PYPPGKECDWKVTVSPDYVIALVFN-IFNLEPGYDFLHIYDGRDSLSPLIGSFYGSQLPG |
| NOV4e | PYPPGKECDWKVTVSPDYVIALVLFPSFNLEPGYDFLHIYDGRDSLSPLIGSFYGSQLPG |
| NOV4f | PYPPGKECDWKVTVSPDYVIALVLFPSFNLEPGYDFLHIYDGRDSLSPLIGSFYGSQLPG |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | RIESSSNSLFLAFRSDASVSNAGFVIDFPENPRESCFDPGSIKNGTRVGSDLKLGSSVTY |
| NOV4b | SVTFECLPGYQLTGHPVLTCQHGTNRNWDHPLPKCEVPCGGNITSSNGTVYSPGFPSPYS |
| NOV4c | RIESSSNSLFLAFRSDASVSNAGFVIDYTENPRESCFDPGSIKNGTRVGSDLKLGSSVTY |
| NOV4d | RIESSSNSLFLAFRSDASVSNAGFVIDYTENPRESCFDPGSIKNGTRVGSDLKLGSSVTY |
| NOV4e | RIESSSNSLFLAFRSDASVSNAGFVIDFPENPRESCFDPGSIKNGTRVGSDLKLGSSVTY |
| NOV4f | RIESSSNSLFLAFRSDASVSNAGFVIDFPENPRESCFDPGSIKNGTRVGSDLKLGSSVTY |
| NOV4g | ------------------------------------------------------------ |
| NOV4h | ------------------------------------------------------------ |
| NOV4i | ------------------------------------------------------------ |
| NOV4j | --------------------------------KLSCFDPGSIKNGTRVGSDLKLGSSVTY |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | YCHGGYEVEGTSTLSCILGPDGKPVWNNPRPVCTAPCGGQYVGSDGVVLSPNYPQNYTSG |
| NOV4b | SSQDCVWLITVPIGHGVRLNLSLLQTEPSGDFITIWDGPQQTAPRLGVFTRSMAKKTVQS |
| NOV4c | YCHGGYEVEGTSTLSCILGPDGKPVWNNPRPVCTAPCGGQYVGSDGVVLSPNYPQNYTSG |
| NOV4d | YCHGGYEVEGTSTLSCILGPDGKPVWNNPRPVCTAPCGGQYVGSDGVVLSPNYPQNYTSG |
| NOV4e | YCHGGYEVEGTSTLSCILGPDGKPVWNNPRPVCTAPCGGQYVGSDGVVLSPNYPQNYTSG |
| NOV4f | YCHGGYEVEGTSTLSCILGPDGKPVWNNPRPVCTAPCGGQYVGSDGVVLSPNYPQNYTSG |
| NOV4g | ------------------------------------------------------------ |
| NOV4h | ------------------------------------------------------------ |
| NOV4i | ------------------------------------------------------------ |
| NOV4j | YCHGGYEVEGTSTLSCILGPDGKPVWNNPRPVCTAPCGGQYVGSDGVVLSPNYPQNYTSG |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | QICLYFVTVPKDYGVVFGQFAFFHTALNDVVEVHDGHSQHSRLLSSLSGSHTG-ESLPLA |
| NOV4b | SSNQVLLKFHRDAATGGIFAIAFSAYPLTKCPPPTILPNAEVVTENEEFNIGD-IVRYRC |
| NOV4c | QICLYFVTVPKDY-VVFGQFAFFHTALNDVVEVHDGHSQHSRLLSSLSGSHTG-ESLPLA |
| NOV4d | QICLYFVTVPKDY-VVFGQFAFFHTALNDVVEVHDGHSQHSRLLSSLSGSHTG-ESLPLA |
| NOV4e | QICLYFVTVPKDYGVVFGQFAFFHTALNDVVEVHDGHSQHSRLLSSLSGSHTG-ESLPLA |
| NOV4f | QICLYFVTVPKDYGVVFGQFAFFHTALNDVVEVHDGHSQHSRLLSSLSGSHTGGESLPLA |
| NOV4g | ------------------------------------------------------------ |
| NOV4h | ------------------------------------------------------------ |
| NOV4i | ------------------------------------------------------------ |
| NOV4j | QICLYFVTVPKDY-VVFGQFAFFHTALNDVVEVHDGHSQHSRLLSSLSGSHTG-ESLPLA |
| NOV4k | ------------------------------------------------------------ |
| Nov4l | ------------------------------------------------------------ |
| NOV4m | ------------------------------------------------------------ |
| NOV4n | ------------------------------------------------------------ |
| NOV4o | ------------------------------------------------------------ |
| NOV4a | TSNQVLIKFSAKGLAPARGFHFVYQAVPRTSATQCSSVPEPRYGKRLGSDFSVGAIVRFE |
| NOV4b | LPGFTLVGNEILTCKLGTYLQFEGPPPICEVHCPTNELLTDSTGVILSQSYPGSYPQFQT |
| NOV4c | TSNQVLIKFSAKGLAPARGFHFVYQAVPRTSATQCSSVPEPRYGKRLGSDFSVGAIVRFE |
| NOV4d | TSNQVLIKFSAKGLAPARGFHFVYQAVPRTSATQCSSVPEPRYGKRLGSDFSVGAIVRFE |
| NOV4e | TSNQVLIKFSAKGLAPARGFHFVYQAVPRTSATQCSSVPEPRYGKPLGSDFSVGAIVRFE |
| NOV4f | TSNQVLIKFSAKGLAPARGFHFVYQAVPRTSATQCSSVPEPRYGKRLGSDFSVGAIVRFE |
| NOV4g | ------------------------------------------------------------ |
| NOV4h | ------------------------------------------------------------ |
| NOV4i | ------------------------------------------------------------ |
| NOV4j | TSNQVLIKFSAKGLAPARGFHFVYQAVPRTSATQCSSVPEPRYGKRLGSDFSVGAIVRFE |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | CNSGYALQGSPEIECLPVPGALAQWNVSAPTCVVPCGGNLTERRGTILSPGFPEPYLNSL |
| NOV4b | CSWLVRVEPDYNISLTVEYFLSEKQYDEFEIFDGPSGQSPLLKALSGNYSAPLIVTSSSN |
| NOV4c | CNSGYALQGSPEIECLPVPGALAQWNVSAPTCVVPCGGNLTERRGTILSPGFPEPYLNSL |
| NOV4d | CNSGYALQGSPEIECLPVPGALAQWNVSAPTCVVPCGGNLTERRGTILSPGFPEPYLNSL |
| NOV4e | CNSGYALQGSPEIECLPVPGAIAQWNVSAPTCVVPCGGNLTERRGTTLSPGFPEPYLNSL |
| NOV4f | CNSGYALQGSPEIECLPVPGALAQWNVSAPTCVVPCGGNLTERRGTILSPGFPEPYLNSL |
| NOV4h | ------------------------------------------------------------ |
| NOV4i | ------------------------------------------------------------ |
| NOV4j | CNSGYALQGSPEIECLPVPGALAQWNVSAPTCVVPCGGNLTERRGTILSPGFPEPYLNSL |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | NCVWKIVVPEGAGIQIQVVSFVTEQNWDSLEVFDGADNTVTMLGSFSGTTVPALLNSTSN |
| NOV4b | SVYLRWSSDHAYNRKGFKIRYSAPYCSLPRAPLHGFILGQTSTQPGGSIHFGCNAGYRLV |
| NOV4C | NCVWKIVVPEGAGIQIQVVSFVTEQNWDSLEVFDGADNTVTMLGSFSGTTVPALLNSTSN |
| NOV4d | NCVWKIVVPEGAGIQIQVVSFVTEQNWDSLEVFDGADNTVTMLGSFSGTTVPALLNSTSN |
| NOV4e | NCVWKIVVPEGAGIQIQVVSFVTEQNWDSLEVFDGADNTVTMLGSFSGTTVPALLNSTSN |
| N0V4f | NCVWKIVVPEGAGIQIQVVSFVTEQNWDSLEVFDGADNTVTMLGSFSGTTVPALLNSTSN |
| NOV4g | ------------------------------------------------------------ |
| NOV4h | ------------------------------------------------------------ |
| NOV4i | ------------------------------------------------------------ |
| NOV4j | NCVWKIVVPEGAGIQIQVVSFVTEQNWDSLEVFDGADNTVTMLGSFSGTTVPALLNSTSN |
| NOV4k | ------------------------------------------------------------ |
| Nov41 | ------------------------------------------------------------ |
| NOV4m | ------------------------------------------------------------ |
| NOV4n | ------------------------------------------------------------ |
| NOV4o | ------------------------------------------------------------ |
| NOV4a | QLYLHFYSDISVSAAGFHLEYKTVGLSSCPEPAVPSNGVKTGERYLVNDVVSFQCEPGYA |
| NOV4b | GHSMAICTRHPQGYHLWSEAIPLCQALSCGLPEAPKNGMVFGKEYTVGTKAVYSCSEGYH |
| NOV4c | QLYLHFYSDISVSAAGFHLEYKTVGLSSCPEPAVPSNGVKTGERYLVNDVVSFQCEPGYA |
| NOV4d | QLYLHFYSDISVSAAGFHLEYKTVGLSSCPEPAVPSNGVKTGERYLVNDVVSFQCEPGYA |
| NOV4e | QLYLHFYSDISVSAAGFHLEYKTVGLSSCPEPAVPSNGVKTGERYLVNDVVSFQCEPGYA |
| NOV4f | QLYLHFYSDISVSAAGFHLEYKTVGLSSCPEPAVPSNGVKTGERYLVNDVVSFQCEPGYA |
| NOV4g | ------------------------------------------------------------ |
| NOV4h | ------------------------------------------------------------ |
| NOV4i | ------------------------------------------------------------ |
| NOV4j | QLYLHFYSDISVSAAGFHLEYKTVGLSSCPEPAVPSNGVKTGERYLVNDVVSFQCEPGYA |
| NOV4k | ------------------------------------------------------------ |
| N0V4l | ------------------------------------------------------------ |
| NOV4m | ------------------------------------------------------------ |
| NOV4n | ---MAICTRHPQGYHLWSEAIPLCQALSCGLPEAPKNGMVFGKEYTVGTKAVYSCSEGYH |
| NOV4o | ---MAICTRHPQGYHLWSEAIPLCQALSCGLPEAPKNGMVFGKEYTVGTKAVYSCSEGYH |
| NOV4a | LQGHAHISCMPGTVRRWNYPPPLCIAQCGGTVEENEGVILSPGFPGNYPSNMDCSWKIAL |
| NOV4b | LQAGAEATAECLDTGLWSNRNVPPQCVPVTCPDVSSISVEHGRWRLIFETQYQFQAQLML |
| NOV4c | LQGHAHISCMPGTVRRWNYPPPLCIAQCGGTVEEMEGVILSPGFPGNYPSNMDCSWKIAL |
| NOV4d | LQGHAHISCMPGTVRRWNYPPPLCIAQCGGTVEEMEGVILSPGFPGNYPSNMDCSWKIAL |
| NOV4e | LQGHAHISCMPGTVRRWNYPPPLCIAQCGGTVEEMEGVILSPGFPGNYPSNNDCSWKIAL |
| NOV4f | LQGHAHISCMPGTVRRWNYPPPLCIAQCGGTVEEMEGVILSPGFPGNYPSNMDCSWKIAL |
| NOV4g | ------------------------------------------------------------ |
| NOV4h | ------------------------------------------------------------ |
| NOV4i | ------------------------------------------------------------ |
| NOV4j | LQGHAHISCMPGTVRRWNYPPPLCIAQCGGTVEENEGVILSPGFPGNYPSNMDCSWKIAL |
| NOV4k | ------------------------------------------------------------ |
| NOV4l | ------------------------------------------------------------ |
| NOV4m | ------------------------------------------------------------ |
| NOV4n | LQAGAEATAECLDTGLWSNRNVPPQCVPVTCPDVSSISVEHGRWRLIFETQYQFQAQLML |
| NOV4o | LQAGAEATAECLDTGLWSNRNVPPQCVPVTCPDVSSISVEHGRWRLIFETQYQFQAQLNL |
| NOV4a | PVGFGAHIQFLNFSTEPNHDYIEIRNGP-------------YETSRMMGRFSGSELPSSL |
| NOV4b | ICDPGYYYTGQRVIRCQANGKWSLGDSTPTCRIISCDELPIPPNGHRIGTLSVYGATAIF |
| NOV4c | PVGFGAHIQFLNFSTEPNHDYIEIRNGP-------------YETSRMNGRFSGSELPSSL |
| NOV4d | PVGFGAHIQFLNFSTEPNHDYIEIRNGP-------------YETSRMMGRFSGSELPSSL |
| NOV4e | PVGFGAHIQFLNFSTEPNHDYIEIRNGP-------------YETSRMMGRFSGSELPSSL |
| NOV4f | PVGFGAHIQFLNFSTEPNHDYIEIRNGP-------------YETSPMMGRFSGSELPSSL |
| NOV4g | ------------------------------------------------------------ |
| NOV4h | ------------------------------------------------------------ |
| NOV4i | ------------------------------------------------------------ |
| NOV4j | PVGFGAHIQFLNFSTEPNHDYIEIRNGP-------------YETSRMMGRFSGSELPSSL |
| NOV4k | ------------------------------------------------------------ |
| NOV4l | ------------------------------------------------------------ |
| NOV4m | ------------------------------------------------------------ |
| NOV4n | ICDPGYYYTGQRVIRCQANGKWSLGDSTPTCRIISCGELPIPPNGHRIGTLSVYGATAIF |
| NOV4o | ICDPGYYYTGQRVIRCQANGKWSLGDSTPTCRIISCGELPIPPNGHRIGTLSVYGATAIF |
| NOV4a | LSTSHETTVYFHSDHSQNRPGFKLEYQAYELQECPDPEPFANGIVRGAGYNVGQSVTFEC |
| NOV4b | SCNSGYTLVGSRVRECMANGLWSGSEVRCLAGHCGTPEPIVNGHINGENYSYRGSVVYQC |
| NOV4c | LSTSHETTVYFHSDHSQNRPGFKLEYQAYELQECPDPEPFANGIVRGAGYNVGQSVTFEC |
| NOV4d | LSTSHETTVYFHSDHSQNRPGFKLEYQAYELQECPDPEPFANGIVRGAGYNVGQSVTFEC |
| NOV4e | LSTSHETTVYFHSDHSQNRPGFKLEYQAYELQECPDPEPFANGIVRGAGYNVGQSVTFEC |
| N0V4f | LSTSHETTVYFHSDHSQNRPGFKLEYQAYELQECPDPEPFANGIVRGAGYNVGQSVTFEC |
| NOV4g | ------------------------------KLECPDPEPFANGIVRGAGYNVGQSVTFEC |
| NOV4h | ------------------------------KLECPDPEPFANGIVRGAGYNVGQSVTFEC |
| NOV4i | ------------------------------KLECPDPEPFANGIVRGAGYNVGQSVTFEC |
| NOV4j | LSTSHETTVYFHSDHSQNRPGFKLEYQAYELQECPDPEPFANGIVRGAGYNVGQSVTFEC |
| NOV4k | ------------------------------------------------------------ |
| NOV4l | ------------------------------------------------------------ |
| NOV4m | ------------------------------------------------------------ |
| NOV4n | SCNSGYTLVGSRVRECMANGLWSGSEVRCLAGHCGTPEPIVNGHINGENYSYRGSVVYQC |
| NOV4o | SCNSGYTLVGSRVRECMANGLWSGSEVRCLAGHCGTPEPIVNGHINGENYSYRGSVVYQC |
| NOV4a | LPGYQLTGHPVLTCQHGTNRN----------WDHPLPKCEVPCGGNITSSNGTVYSPGFP |
| NOV4b | NAGFRLIGMSVRICQQDHHWSGKTPFCVPITCGHPGNPVNGLTQGNQFNLNDVVKFVCNP |
| NOV4c | LPGYQLTGHPVLTCQHGTNRN----------WDHPLPKCEVPCGGNITSSNGTVYSPGFP |
| NOV4d | LPGYQLTGHPVLTCQHGTNRN----------WDHPLPKCEVPCGGNITSSNGTVYSPGFP |
| NOV4e | LPGYQLTGHPVLTCQHGTNRN----------WDHPLPKCEVPCGGNITSSNGTVYSPGFP |
| NOV4f | LPGYQLTGHPVLTCQHGTNRN----------WDHPLPKCEVPCGGNITSSNGTVYSPGFP |
| NOV4g | LPGYQLTGHPVLTCQHGTNRN----------WDHSLPKCEVPCGGNITSSNGTVYSPGFP |
| NOV4h | LPGYQLTGHPVLTCQHGTNRN----------WDHPLPKCEVPCGGNITSSNGTVYSPGFP |
| NOV4i | LPGYQLTGHPVLTCQHGTNRN----------WDHPLPKCEVPCGGNITSSNGTVYSPGFP |
| NOV4j | LPGYQLTGHPVLTCQHGTNRN----------WDHPLPKCEVPCGGNITSSNGTVYSPGFP |
| NOV4k | ------------------------------------------------------------ |
| NOV4l | ------------------------------------------------------------ |
| NOV4m | ------------------------------------------------------------ |
| NOV4n | NAGFRLIGMSVRICQQDHHWSGKTPFCVPITCGHPGNPVNGLTQGNQFNLNDVVKFVCNP |
| NOV4o | NAGFRLIGMSVRICQQDHHWSGKTPFCVPITCGHPGNPVNGLTQGNQFNLNDVVKFVCNP |
| NOV4a | SPYSSSQDCVWLITVPIGHGVRLNLSLLQTEPSGDFITIWDGPQQTAPRLGVFTRSMAKK |
| NOV4b | GYMAEGAARSQCLASGQWSDMLPTCRIINCTDPGHQENSVRQVHASGPHRFSFGTTVSYR |
| NOV4c | SPYSSSQDCVWLITVPIGHGVRLNLSLLQTEPSGDFITIWDGPQQTAPRLGVFTRSMAKK |
| NOV4d | SPYSSSQDCVWLITVPIGHGVRLNLSLLQTEPSGDFITIWDGPQQTAPRLGVFTRSMAKK |
| NOV4e | SPYSSSQDCVWLITVPIGHGVRLNLSLLQTEPSGDFITIWDGPQQTAPRLGVFTRSMAKK |
| NOV4f | SPYSSSQDCVWLITVPIGHGVRLNLSLLQTEPSGDFITIWDGPQQTAPRLGVFTRSMAKK |
| NOV4g | SPYSSSQDCVWLITVPIGHGVRLNLSLLQTEPSGDFITIWDGPQQTAPRLGVFTRSMAKK |
| NOV4h | SPYSSSQDCVWLITVPIGHGVRLNLSLLQTEPSGDFITIWDGPQQTAPRLGVFTRSMAKK |
| NOV4i | SPYSSSQDCVWLITVPIGHGVRLNLSLLQTEPSGDFITIWDGPQQTAPRLGVFTRSMAKK |
| NOV4j | SPYSSSQDCVWLITVPIGHGVRLNLSLLQTEPSGDFITIWDGPQQTAPRLGVFTRSMAKK |
| NOV4k | ------------------------------------------------------------ |
| NOV4l | ---AEGAARSQCLASGQWSDMLPTCRIINCTDPGHQENSVRQVHASGPHRFSFGTTVSYR |
| NOV4m | ----------------------------------------------------MGYTLIYS |
| NOV4n | GYMAEGAARSQCLASGQWSDMLPTCRIINCTDPGHQENSVRQVHASGPHRFSFGTTVSYR |
| NOV4o | GYMAEGAARSQCLASGQWSDMLPTCRIINCTDPGHQENSVRQVHASGPHRFSFGTTVSYR |
| NOV4a | TVQSSSNQVLLKFHRDAATGGIFAIAFSAYPLTKCPPPTILPNAEVVTENEEFNIGDIVR |
| NOV4b | C--NHGFYLLGTPVLSCQGDGTWDRPRPQCLLVSCGHPGSPPHSQMSGDS--YTVGAVVR |
| NOV4c | TVQSSSNQVLLKFHRDAATGGIFAIAFSAYPLTKCPPPTILPNAEVVTENEEFNIGDIVR |
| NOV4d | TVQSSSNQVLLKFHRDAATGGIFAIAFSAYPLTKCPPPTILPNAEVVTENEEFNIGDIVR |
| NOV4e | TVQSSSNQVLLKFHRDAATGGIFAIAFSAYPLTKCPPPTILPNAEVVTENEEFNIGDIVR |
| NOV4f | TVQSSSNQVLLKFHRDAATGGIFAIAFSAYPLTKCPPPTILPNAEVVTENEEFNIGDIVR |
| NOV4g | TVQSSSNQVLLKFHRDAATGGIFAIAFSAYPLTKCPPPTILPNAEVVTENEEFNIGDIVR |
| NOV4h | TVQSSSNQVLLKFHRDAATGGIFAIAFSAYPLTKCPPPTILPNAEVVTENEEFNIGDIVR |
| NOV4i | TVQSSSNQVLLKFHRDAATGGIFAIAFSAYPLTKCPPPTTLPNAEVVTENEEFNIGDIVR |
| NOV4j | TVQSSSNQVLLKFHRDAATGGIFAIAFSAYPLTKCPPPTILPNAEVVTENEEFNIGDIVR |
| NOV4k | ------------------------------------------------------------ |
| NOV4l | C--NHGFYLLGTPVLSCQGDGTWDRPRPQCLLVSCGHPGSPPHSQMSGDS--YTVGAVVR |
| NOV4m | C-QEGFSLKGGSEHRTCKADGSWTGKPPICLEVR---PSGRPINTAREPP--------LT |
| NOV4n | CX-TTASTSWATPVLSCQGDGTWDRPRPQCLLVSCGHPGSPPHSQMSGDS--YTVGAVVR |
| NOV4o | C--NHGFYLLGTPVLSCQGDGTWDRPRPQCLLVSCGHPGSPPHSQMSGDS--YTVGAVVR |
| NOV4a | YRCLPGFTLVGNEILTCKLGTYLQFEGPPPICEVHCPTNELLTDSTGVILSQSYPGSYPQ |
| NOV4b | YSCIGKRTLVGNSTRMCGLDGHWTGSLPHCSGTSVGVCGDPGIPAHGIRLGDSFDPGTVM |
| NOV4c | YRCLPGFTLVGNEILTCKLGTYLQFEGPPPICEVHCPTNELLTDSTGVILSQSYPGSYPQ |
| NOV4d | YRCLPGFTLVGNEILTCKLGTYLQFEGPPPICEVHCPTNELLTDSTGVILSQSYPGSYPQ |
| NOV4e | YRCLPGFTLVGNEILTCKLGTYLQFEGPPPICEVHCPTNELLTDSTGVILSQSYPGSYPQ |
| NOV4f | YRCLPGFTLVGNEILTCKLGTYLQFEGPPPICEVHCPTNELLTDSTGVILSQSYPGSYPQ |
| NOV4g | YRCLPGFTLVGNEILTCKLGTYLQFEGPPPICEVHCPTNELLTDSTGVILSQSYPGSYPQ |
| NOV4h | YRCLPGFTLVGNEILTCKLGTYLQFEGPPPICEVHCPTNELLTDSTGVILSQSYPGSYPQ |
| NOV4i | YRCLPGFTLVGNEILTCKLGTYLQFEGPPPICEVHCPTNELLTDSTGVILSQSYPGSYPQ |
| NOV4j | YRCLPGFTLVGNEILTCKLGTYLQFEGPPPICEVHCPTNELLTDSTGVILSQSYPGSYPQ |
| NOV4k | ------------------------------------------------------------ |
| NOV4l | YSCIGKRTLVGNSTRMCGLDGHWTGSLPHCSGTSVGVCGDPGIPAHGIRLGDSFDPGTVM |
| NOV4m | QALIPGDVFAKNSLWKGAYEYQGKKQ-PAMLRVTGFQVANSKVNATMIDHSG-VELHLAG |
| NOV4n | YSCIGKRTLVGNSTRMCGLDGHWTGSLPHCSGTSVGVCGDPGIPAHGIRLGDSFDPGTVM |
| NOV4o | YSCIGKRTLVGNSTRMCGLDGHWTGSLPHCSGTSVGVCGDPGIPAHGIRLGDSFDPGTVN |
| NOV4a | FQTCSWLVRVEPDYNISLTVEYFLSEKQYDEFEIFDGPSGQSPLLKALSGNYSAPLIVTS |
| NOV4b | RFSCEAGHVLRGSSERTCQANGSWSGSQPECGVISCGNPG--------TPSNARVVFSDG |
| NOV4c | FQTCSWLVRVEPDYNISLTVEYFLSEKQYDEFEIFDGPSGQSPLLKALSGNYSAPLIVTS |
| NOV4d | FQTCSWLVRVEPDYNISLTVEYFLSEKQYDEFEIFDGPSGQSPLLKALSGNYSAPLIVTS |
| NOV4e | FQTCSWLVRVEPDYNISLTVEYFLSEKQYDEFEIFDGPSGQSPLLKALSGNYSAPLIVTS |
| NOV4f | FQTCSWLVRVEPDYNISLTVEYFLSEKQYDEFEIFDGPSGQSPLLKALSGNYSAPLIVTS |
| NOV4g | FQTCSWLVRVEPDYNISLTVEYFLSEKQYDEFEIFDGPSGQSPLLKALSGNYSAPLIVTS |
| NOV4h | FQTCSWLVRVEPDYNISLTVEYFLSEKQYDEFEIFDGPSGQSPLLKALSGNYSAPLIVTS |
| NOV4i | FQTCSWLVRVEPDYNISLTVEYFLSEKQYDEFEIFDGPSGQSPLLKALSGNYSAPLIVTS |
| NOV4j | FQTCSWLVRVEPDYNISLTVEYFLSEKQYDEFEIFDGPSGQSPLLKALSGNYSAPLIVTS |
| NOV4k | ------------------------------------------------------------ |
| NOV4l | RFSCEAGHVLRGSSERTCQANGSWSGSQPECGVISCGNPG--------TPSNARVVFSDG |
| NOV4m | TYKKEDFHLLLQVYQITGPVEIFMN-KFKDDHWALDGHVS--------SANSPPPLTPRS |
| NOV4n | RFSCEAGHVLRGSSERTCQANGSWSGSQPECGVISCGNPG--------TPSNARVVFSDG |
| NOV4o | RFSCEAGHVLRGSSERTCQANGSWSGSQPECGVISCGNPG--------TPSNARVVFSDG |
| NOV4a | SSNSVYLRWSSDHAYNRKGFKIRYSAPYCSLPRAPLHGFILGQTSTQPGGSIHFGCNAGY |
| NOV4b | LVFSSSIVYECREGYYATGLLSRHCSVNGTWTGS----------D--------------- |
| NOV4c | SSNSVYLRWSSDHAYNRKGFKIRYSAPYCSLPRAPLHGFILGQTSTQPGGSIHFGCNAGY |
| NOV4d | SSNSVYLRWSSDHAYNRKGFKIRYSALSCGLP---------------------------- |
| NOV4e | SSNSVYLRWSSDHAYNRKGFKIRYSALSCGLPEAPKNGMVFGKEYTVGTKAMYSCSEGYH |
| NOV4f | SSNSVYLRWSSDHAYNRKGFKIRYSAPYCSLPRAPLHGFILGQTSTQPGGSIHFGCNAGY |
| NOV4g | SSNSVYLRWSSDHAYNRKGFKIRYSAPYCSLPRAPLHGFILGQTSTQPGGSIHFGCNAGY |
| NOV4h | SSNSVYLRWSSDHAYNRKGFKIRYSAPYCSLPRAPLHGFILGQTSTQPGGSIHFGCNAGY |
| NOV4i | SSNSVYLRWSSDHAYNRKGFKIRYSA---------------------------------- |
| NOV4j | SSNSVYLRWSSDHAYNRKGFKIRYVD---------------------------------- |
| NOV4k | ------------------------------------------------------------ |
| NOV4l | LVFSSSIVYECREGYYATGLLSRHCSVNGTWTGS----------D--------------- |
| NOV4m | LAGKRRTKGCANP----------------------------------------------- |
| NOV4n | LVFSSSIVYECREGYYATGLLSRHCSVNGTWTGS----------D--------------- |
| NOV4o | LVFSSSIVYECREGYYATGLLSRHCSVNGTWTGS----------D--------------- |
| NOV4a | RLVGHSMAICTRHPQGYH------------------------------------------ |
| NOV4b | ------------------------------------------------------------ |
| NOV4c | RLVGHSMAICTRHPQGYH------------------------------------------ |
| NOV4d | ------------------------------------------------------------ |
| NOV4e | LQAGAEATAECLDTGLWSNRNVPPQCVPVTCPDVSSISVEHGRWRLIFETQYQFQAQLML |
| NOV4f | RLVGHSMAICTRHPQGYHLWSEAIPLCQALSCGLPEAP---------------------- |
| NOV4g | RLVGHSMAICTRHPQGYH------------------------------------------ |
| NOV4h | RLVGHSMAICTRHPQGYH------------------------------------------ |
| NOV4i | ---------LS------------------------------------------------- |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | ------------------------------------------------------------ |
| NOV4b | ------------------------------------------------------------ |
| NOV4c | ------------------------------------------------------------ |
| NOV4d | ------------------------------------------------------------ |
| NOV4e | ICDPGYYYTGQRVIRCQANGKWSLGDSTPTCRIISCGELPIPPNGHRIGTLSVYGATAIF |
| NOV4f | ------------------------------------KNGMVFGKEYTVGTKAMYSCSEGY |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | ------------------------------------------------------------ |
| NOV4b | ------------------------------------------------------------ |
| NOV4c | ------------------------------------------------------------ |
| NOV4d | ------------------------------------------------------------ |
| NOV4e | SCNSGYTLVGSRVRECMANGLWSGSEVRCLAGHCGTPEPIVNGHINGENYSYRGSVTYQC |
| NOV4f | HLQAGAEATAECLDTGLWSNRNVPPQCVPVTCPDVSSISVEHGRWRLIFETQYQFQAQLM |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | ------------------------------------------------------------ |
| NOV4b | ------------------------------------------------------------ |
| NOV4c | ------------------------------------------------------------ |
| NOV4d | ------------------------------------------------------------ |
| NOV4e | NAGFRLIGMSVRICQQDHHWSGKTPFCVPITCGHPGNPVNGLTQGNQFNLNDVVKFVCNP |
| N0V4f | LICDPGYYYTGQRVIRCQANGKWSLGDSTPTCRIISCGELPIPPNGHRIGTLSVYGATAI |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | ------------------------------------------------------------ |
| NOV4b | ------------------------------------------------------------ |
| NOV4c | ------------------------------------------------------------ |
| NOV4d | ------------------------------------------------------------ |
| NOV4e | GYMAEGAARSQCLASGQWSDMLPTCRIINCTDPGHQENSVRQVHASGPHRFSFGTTVSYR |
| NOV4f | FSCNSGYTLVGSRVRECMANGLWSGSEVRCLAGHCGTPEPIVNGHINGENYSYRGSVVYQ |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | ------------------------------------------------------------ |
| NOV4b | ------------------------------------------------------------ |
| NOV4c | ------------------------------------------------------------ |
| NOV4d | ------------------------------------------------------------ |
| NOV4e | CNHGFYLLGTPVLSCQGDGTWDRPRPQCLLVSCGHPGSPPHSQMSGDSYTVGAVVRYSCI |
| NOV4f | CNAGFRLIGMSVRICQQDHHWSGKTPFCVLVSCGHPGSPPHSQMSGDSYTVGAVVRYSCI |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| Nov4l | ------------------------------------------------------------ |
| NOV4m | ------------------------------------------------------------ |
| NOV4n | ------------------------------------------------------------ |
| NOV4o | ------------------------------------------------------------ |
| NOV4a | ------------------------------------------------------------ |
| NOV4b | ------------------------------------------------------------ |
| NOV4c | ------------------------------------------------------------ |
| NOV4d | ------------------------------------------------------------ |
| NOV4e | GKRTLVGNSTRMCGLDGHWTGSLPHCSGTSVGVCGDPGIPAHGIRLGDSFDPGTVMRFSC |
| NOV4f | GKRTLVGNSTRMCGLDGHWTGSLPHCSGTSVGVCGDPGIPAHGIRLGDSFDPGTVMRFSC |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4o | ------------------------------------------------------------ |
| NOV4a | ------------------------------------------------------------ |
| NOV4b | ------------------------------------------------------------ |
| NOV4c | ------------------------------------------------------------ |
| NOV4d | ------------------------------------------------------------ |
| NOV4e | EAGHVLRGSSERTCQANGSWSGSQPECGVISCGNPGTPSNARVVFSDGLVFSSSIVYECR |
| NOV4f | EAGHVLRGSSERTCQANGSWSGSQPECGVISCGNPGTPSNARVVFSDGLVFSSSIVYECR |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | -----------------LWSEAIPLCQALSCGLPEAPKNGMVFGKEYTVGTKAMYSCSEG |
| NOV4b | -----------------------PECLVINCGDPGIPANGLRLGNDFRYNKTVTYQCVPG |
| NOV4c | -----------------LWSEAIPLCQALSCGLPEAPKNGMVFGKEYTVGTKAMYSCSEG |
| NOV4d | ----------------------------------EAPKNGMVFGKEYTVGTKAMYSCSEG |
| NOV4e | EGYYATGLLSRHCSVNGTWTGSDPECLVINCGDPGIPANGLRLGNDFRYNKTVTYQCVPG |
| NOV4f | EGYYATGLLSRHCSVNGTWTGSDPECLVINCGDPGIPANGLRLGNDFRYNKTVTYQCVPG |
| NOV4g | -----------------LWSEAIPLCQALSCGLPEAPKNGMVFGKEYTVGTKAMYSCSEG |
| NOV4h | -----------------LWSEAIPLCQALSCGLPEAPKNGMVFGKEYTVGTKAVYSCSEG |
| NOV4i | ------------------------------CGLPEAPKNGMVFGKEYTVGTKAMYSCSEG |
| NOV4j | ------------------------------------------------------------ |
| NOV4k | ------------------------------------------------------------ |
| NOV4l | -----------------------PECLVINCGDPGIPANGLRLGNDFRYNKTVTYQCVPG |
| NOV4m | ------------------------------------------------------------ |
| NOV4n | -----------------------PECLVINCGDPGIPANGLRLGNDFRYNKTVTYQCVPG |
| NOV4o | -----------------------PECLVINCGDPGIPANGLRLGNDFRYNKTVTYQCVPG |
| NOV4a | YHLQAGAEATAECLDTGLWSNRNVPPQCVP---------VTCPDVSSISVEHGRWRLIFE |
| NOV4b | YMMESHRVSVLSCTKDRThNGTKPVCKALMCK--------PPPLI--------PNGKVVG |
| NOV4c | YHLQAGAEATAECLDTGLWSNRNVPPQCVRESSGNGGGSVTCPDVSSISVEHGRWRLIFE |
| NOV4d | YHLQAGAEATAECLDTGLWSNRNVPPQCVP---------VTCPDVSSISVEHGRWRLIFE |
| NOV4e | YMMESHRVSVLSCTKDRTWNGTKPVCKALMCK--------PPPLIP--------NGKVVG |
| NOV4f | YMMESHRVSVLSCTKDRTWNGTKPVCKALMCK--------PPPLIP--------NGKVVG |
| NOV4g | YHLQAGAEATAECLDTGLWSNRNVPPQCVP---------VTCPDVSSISVEHGRWRLIFE |
| NOV4h | YHLQAGAEATAECLDTGLWSNRNVPPQCVP---------VTCPDVSSISVEHGRWRLIFE |
| NOV4i | YHLQAGAEATAECLDTGLWSNPNVPPQCVP---------VTCPDVSSISVEHGRWRPIFE |
| NOV4j | ------------------------------------------------------------ |
| NOV4k | ------------------------------------------------------------ |
| Nov4l | YMMESHRVSVLSCTKDRTWNGTKPVCKALMCK--------PPPLI--------PNGKVVG |
| NOV4m | ------------------------------------------------------------ |
| NOV4n | YMMESHRVSVLSCTKDRTWNGTKPVCKALMCK--------PPPLI--------PNGKVVG |
| NOV4o | YMMESHRVSVLSCTKDRTWNGTKPVCKALMCK--------PPPLI--------PNGKVVG |
| NOV4a | TQYQFQAQLMLICDPGYYYTGQRVIRCQANGKWSLGDSTPTCRIISCGELPIPPNGHRIG |
| NOV4b | SDFMWGSSVTYACLEGYQLSLPAVFTCEGNGSWTG--ELPQCFPVFCGDPGVPSRGRRED |
| NOV4c | TQYQFQAQLMLICDPGYYYTGQRVIRCQANGKWSLGDSTPTCRIISCGELPIPPNGHRIG |
| NOV4d | TQYQFQAQLMLICDPGYYYTGQRVIRCQMJGKWSLGDSTPTCRIISCGELPIPPNGHRIG |
| NOV4e | SDFMWGSSVTYACLEGYQLSLPAVFTCEGNGSWTG--ELPQCFPVFCGDPGVPSRGRRED |
| NOV4f | SDFMWGSSVTYACLEGYQLSLPAVFTCEGNGSWTG--ELPQCFPVFCGDPGVPSRGRRED |
| NOV4g | TQYQFQAQLMLICDPGYYYTGQRVIRCQANGKWSLGDSTPTCRIISCGELPIPPNGHRIG |
| NOV4h | TQYQFQAQLMLICDPGYYYTGQRVIRCQANGKWSLGDSTPTCRIISCGELPIPPNGHRIG |
| NOV4i | TQYQFQAQLMLICDPGYYYTGQRVIRCQANGKWSLGDSTPTCRIISCGELPIPPNGHRIG |
| NOV4j | ------------------------------------------------------------ |
| NOV4k | ------------------------------------------------------------ |
| NOV4l | SDFMWGSSVTYACLEGYQLSLPAVFTCEGNGSWTG--ELPQCFPVFCGDPGVPSRGRRED |
| NOV4m | ------------------------------------------------------------ |
| NOV4n | SDFMWGSSVTYACLEGYQLSLPAVFTCEGNGSWTG--ELPQCFPVFCGDPGVPSRGRRED |
| NOV4o | SDFMWGSSVTYACLEGYQLSLPAVFTCEGNGSWTG--ELPQCFPVFCGDPGVPSRGRRED |
| NOV4a | TLSVYGATAIFSCNSGYTLVGSRVRECMANGLWSGS------------------------ |
| NOV4b | RGFSYRSSVSFSCHPPLVLVGSPRRFCQSDGTWSGTQP---------------------S |
| NOV4c | TLSVYGATAIFSCNSGYTLVGSRVRECMANGLWSGSEVRCLATQTKLHSIFYKLLFDVLS |
| NOV4d | TLSVYGATAIFSCNSGYTLVGSRVRECMANGLWSGS------------------------ |
| NOV4e | RGFSYRSSVSFSCHPPLVLVGSPRRFCQSDGTWSGTQP---------------------S |
| NOV4f | RGFSYRSSVSFSCHPPLVLVGSPRRFCQSDGTWSGTQP---------------------S |
| NOV4g | TLSVYGATATFSCNSGYTLVGSRVRECMANGLWSGS------------------------ |
| NOV4h | TLSVYGATAIFSCNSGYTLVGSRVRECMANGLWSGS------------------------ |
| NOV4i | TLSVYGATAIFSCNSGYTLVGSRVRECMANGLWSGS------------------------ |
| NOV4j | ------------------------------------------------------------ |
| NOV4k | -----KSSVSFSCHPPLVLVGSPRLRFCQSDGTWSGTQP--------------------S |
| NOV4l | RGFSYRSSVSFSCHPPLVLVGSPRLRFCQSDGTWSGTQP--------------------- |
| NOV4m | ------------------------------------------------------------ |
| NOV4n | RGFSYRSSVSFSCHPPLVLVGSPRRFCQSDGTWSGTQP---------------------S |
| NOV4o | RGFSYRSSVSFSCHPPLVLVGSPRRFCQSDGTWSGTQP---------------------S |
| NOV4a | -EVRCLAGHCGTPEPIVNGHINGENYSYRGSVVYQCNAGFRLIGMSVRICQQDHHWSGKT |
| NOV4b | CIDPTLTTCADPGVPQFGIQNNSQGYQVGSTVLFRCQKGYLLQGSTTRTCLPNLTWSGTP |
| NOV4c | SPSLTKAGHCGTPEPIVNGHINGENYSYRGSVVYQCNAGFRLIGMSVRICQQDHHWSGKT |
| NOV4d | -EVRCLAGHCGTPEPIVNGHINGENYSYRGSVVYQCNAGFRLIGMSVRTCQQDHHWSGKT |
| NOV4e | CIDPTLTTCADPGVPQFGIQNNSQGYQVGSTVLFRCQKGYLLQGSTTRTCLPNLTWSGTP |
| NOV4f | CIDPTLTTCADPGVPQFGIQNNSQGYQVGSTVLFRCQKGYLLQGSTTRTCLPNLTWSGTP |
| NOV4g | -EVRCLAGHCGTPEPIVNGHINGENYSYRGSVVYQCNAGFRLIGMSVRTCQQDHHWSGKT |
| NOV4h | -EVRCIAGHCGTPEPIVNGHINGENYSYRGSVVYQCNAGFRLIGMSVRICQQDHHWSGKT |
| NOV4i | -EVRCLAGHCGTPEPIVNGHINGENYSYRGSVVYQCNAGFRLIGMSVRICQQDHHWSGKT |
| NOV4j | ------------------------------------------------------------ |
| NOV4k | CIDPTLTTCADPGVPQFGIQNNSQGYQVGSTVLFRCQKGYLLQGSTTRTCLPNLTWSGTP |
| NOV4l | CIDPTLTTCADPGVPQFGIQNNSQGYQVGSTVLFRCQKGYLLQGSTTRTCLPNLTWSGTP |
| NOV4m | ------------------------------------------------------------ |
| NOV4n | CIDPTLTTCADPGVPQFGIQNNSQGYQVGSTVLFRCQKGYLLQGSTTRTCLPNLTWSGTP |
| NOV4o | CIDPTLTTCADPGVPQFGIQNNSQGYQVGSTVLFRCQKGYLLQGSTTRTCLPNLTWSGTP |
| NOV4a | PFCVPITCGHPGNPVNGLTQGNQFNLNDVVKFVCNPGYMAEG--AARSQCLASGQWSDML |
| NOV4b | PDCVPHHCRQPETPTHANVGALDLPSMGYTLITPARRASPSR--VAPSTAPARRMAAGQA |
| NOV4c | PFCVHVKQQLLLLLLLLCDDD----DDE-------------------------------- |
| NOV4d | PFCQLPVDTQATLSTASLRVTS-LTSTMWSSLFATLGIWLRG--LLGPNAWPAGNGVTCC |
| NOV4e | PDCVPHHCRQPETPTHANVGALDLPSMGYTLIYSCQEGFSLKGGSEHRTCKADGSWTGKP |
| NOV4f | PDCVPHHCRQPETPTHANVGALDLPSMGYTLIYSCQEGFSLKGGSEHRTCKADGSWTGKP |
| NOV4g | PFCVPITCGHPGNPVNGLTQGNQFNLNDVVKFVCNPGYMAEG--AARSQCLASGQWSDML |
| NOV4h | PFCVPITCGHPGNPVNGLTQGNQFNLNDVVKFVCNPGYMAEG--AARSQCLASGQWSDML |
| NOV4i | PFCVPITCGHPGNPVNGLTQGNQFNLNDVVKFVCNPGYMAEG--AARSQCLASGQWSDML |
| NOV4j | ------------------------------------------------------------ |
| NOV4k | PDCVP------------------------------------------------------- |
| NOV4l | PDCVPHHCRQPETPTHANVGALDLPSMGYTLIYSCQEGFSLKGGSEHRTCKADGSWTGKP |
| NOV4m | ------------------------------------------------------------ |
| NOV4n | PDCVPHHCRQPETPTHANVGALDLPSMGYTLITPARRASPSR--VAPSTAPARRMAAGQA |
| NOV4o | PDCVPHHCRQPETPTHANVGALDLPSMGYTLITPARRASPSR--VAPSTAPARRMAAGQA |
| NOV4a | PTCRIINCTDPGHQENSVRQVHASGPHRFSFGTTVSYRCNHGFYLLGTPVLSCQGDGTWD |
| NOV4b | SRPSAWR-SGPVGDPSTLPGSHRSPKP--------------------------------- |
| NOV4c | -DDGSGAITCGHPGNPVNGLTQGNQFNLNDVVKFVCNPGYMAEGAARSQCLASGQWSDML |
| NOV4d | PPAESSTVQILDTKKIVFVRSTPAARTGSASAPLCLTGATTASTSWAPQCSAARENAHGT |
| NOV4e | PICLEVRPNGRPINTAREPPLTQALIPGDVFAKNSLWKGAYEYQGKKQPAMLRVTGFQVA |
| NOV4f | PICLEVRPNGRPINTAREPPLTQALIPGDVFAKNSLWKGAYEYQGKKQPAMLRVTGFQVA |
| NOV4g | PTCRIINCTDPGHQENSVRQVHASGPHRFSFGTTVSYRCNHGFYLLGTPVLSCQGDGTWD |
| NOV4h | PTCRIINCTDPGHQENSVRQVHASGPHRFSFGTTVSYRCNHGFYLLGTPVLSCQGDGTWD |
| NOV4i | PTCRIINCTDPGHQENSVRQVHASGPHRFSFGTTVSYRCNHGFYLLGTPVLSCQGDGTWD |
| NOV4j | ------------------------------------------------------------ |
| NOV4k | ------------------------------------------------------------ |
| Nov4l | PICLEVRPSGRPINIAREPPLTQALIPGDVFAKNSLWKG--------------------- |
| NOV4m | ------------------------------------------------------------ |
| NOV4n | SRPSAWQRSGPVGDPSTLPGSHRSPKP--------------------------------- |
| NOV4o | SRPSAWR-SGPVGDPSTLPGSHRSPKP--------------------------------- |
| NOV4a | RPRPQCLLVSCGHPGSPPHSQMSGDSYTVGAVVRYSCIGKRTLVGNSTRMCGLDGHWTGS |
| NOV4b | ------------------------------------------------------------ |
| NOV4c | PTCRIINCTDPGHQENSVRQVHASGPHRFSFGTTVSYRCNHGFYLLGTPVLSCQGDGTWD |
| NOV4d | VPAPSVSVSSGVLWPSGLPASLPDVWRQLYCGSSGAVQLHRQAYSGGKQHPHVWAGWTLD |
| NOV4e | NSKVNATMIDHSGVELHLAGTYKKEDFHLLLQVYQITGPVEIFMNKFKDDHWALDGHVSS |
| NOV4f | NSKVNATMIDHSGVELHLAGTYKKEDFHLLLQVYQITGPVEIFMNKFKDDHWALDGHVSS |
| NOV4g | RPRPQCLCKVD------------------------------------------------- |
| NOV4h | RPRPQCLCKVD------------------------------------------------- |
| NOV4i | RPRPQCLCKVD------------------------------------------------- |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | LPHCSGTSVGVCGDPGIPAHGIRLGDSFDPGTVMRFSCEAGHVLRGSSERTCQANGSWSG |
| NOV4b | ------------------------------------------------------------ |
| NOV4c | RPRPQCLCK--------------------------------------------------- |
| NOV4d | WLPPSLLRNQRGSLR--------------------------------------------- |
| NOV4e | ESSGATFIYQGSVKGQGFGQFGFQRLDLRLLESDPESIGRHFASNSSSVAAAILVPFIAL |
| NOV4f | ESSGATFIYQGSVKGQGFGQFGFQRLDLRLLESDPESIGRHFASNSSSVAAAILVPFIAL |
| NOV4g | ------------------------------------------------------------ |
| NOV4h | ------------------------------------------------------------ |
| NOV4i | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | SQPECGVISCGNPGTPSNARVVFSDGLVFSSSIVYECREGYYATGLLSRHCSVNGTWTGS |
| NOV4b | ------------------------------------------------------------ |
| NOV4c | ------------------------------------------------------------ |
| NOV4d | ------------------------------------------------------------ |
| NOV4e | IIAGFVLYLYKHRRRPKVPFNGYAGHENTNVPATFENPMYDRNIQPTDIMASEAEFTVST |
| NOV4f | IIAGFVLYLYKHRRRPKVPFNGYAGHENTNVRATFENPMYDRNIQPTDIMASEAEFTVST |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | DPECLVINCGDPGIPANGLRLGNDFRYNKTVTYQCVPGYMMESHRVSVLSCTKDRTWNGT |
| NOV4b | ------------------------------------------------------------ |
| NOV4c | ------------------------------------------------------------ |
| NOV4d | ------------------------------------------------------------ |
| NOV4e | VCTAV------------------------------------------------------- |
| NOV4f | VCTAV------------------------------------------------------- |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| Nov4l | ------------------------------------------------------------ |
| NOV4m | ------------------------------------------------------------ |
| NOV4n | ------------------------------------------------------------ |
| NOV4o | ------------------------------------------------------------ |
| NOV4a | KPVCKALMCKPPPLIPNGKVVGSDFMWGSSVTYACLEGYQLSLPAVFTCEGNGSWTGELP |
| NOV4b | ------------------------------------------------------------ |
| NOV4c | ------------------------------------------------------------ |
| NOV4d | ------------------------------------------------------------ |
| NOV4e | ------------------------------------------------------------ |
| NOV4f | ------------------------------------------------------------ |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | QCFPVFCGDPGVPSRGRREDRGFSYRSSVSFSCHPPLVLVGSPRRFCQSDGTWSGTQPSC |
| NOV4b | ------------------------------------------------------------ |
| NOV4c | ------------------------------------------------------------ |
| NOV4d | ------------------------------------------------------------ |
| NOV4e | ------------------------------------------------------------ |
| NOV4f | ------------------------------------------------------------ |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | IDPTLTTCADPGVPQFGIQNNSQGYQVGSTVLFRCQKGYLLQGSTTRTCLPNLTWSGTPP |
| NOV4b | ------------------------------------------------------------ |
| NOV4c | ------------------------------------------------------------ |
| NOV4d | ------------------------------------------------------------ |
| NOV4e | ------------------------------------------------------------ |
| NOV4f | ------------------------------------------------------------ |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | DCVPHHCRQPETPTHANVGALDLPSMGYTLIYSCQEGFSLKGGSEHRTCKADGSWTGKPP |
| NOV4b | ------------------------------------------------------------ |
| NOV4c | ------------------------------------------------------------ |
| NOV4d | ------------------------------------------------------------ |
| NOV4e | ------------------------------------------------------------ |
| NOV4f | ------------------------------------------------------------ |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| Nov4l | ------------------------------------------------------------ |
| NOV4m | ------------------------------------------------------------ |
| NOV4n | ------------------------------------------------------------ |
| NOV4o | ------------------------------------------------------------ |
| NOV4a | ICLEVRPNGRPINTAREPPLTQALIPGDVFAKNSLWKGAYEYQGKKQPAMLRVTGFQVAN |
| NOV4b | ------------------------------------------------------------ |
| NOV4c | ------------------------------------------------------------ |
| NOV4d | ------------------------------------------------------------ |
| NOV4e | ------------------------------------------------------------ |
| NOV4f | ------------------------------------------------------------ |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| Nov4l | ------------------------------------------------------------ |
| NOV4m | ------------------------------------------------------------ |
| NOV4n | ------------------------------------------------------------ |
| NOV4o | ------------------------------------------------------------ |
| NOV4a | SKVNATMIDHSGVELHLAGTYKKEDFHLLLQVYQITGPVEIFMNKFKDDHWALDGHVSSE |
| NOV4b | ------------------------------------------------------------ |
| NOV4c | ------------------------------------------------------------ |
| NOV4d | ------------------------------------------------------------ |
| NOV4e | ------------------------------------------------------------ |
| NOV4f | ------------------------------------------------------------ |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | SSGATFIYQGSVKGQGFGQFGFQRLDLRLLESDPESIGRBFASNSSSVAAAILVPFIALI |
| NOV4b | ------------------------------------------------------------ |
| NOV4c | ------------------------------------------------------------ |
| NOV4d | ------------------------------------------------------------ |
| NOV4e | ------------------------------------------------------------ |
| NOV4f | ------------------------------------------------------------ |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | IAGFVLYLYKHRRRPKVPFNGYAGHENTNVRATFENPMYDRNIQPTDIMASEAEFTVSTV |
| NOV4b | ------------------------------------------------------------ |
| NOV4c | ------------------------------------------------------------ |
| NOV4d | ------------------------------------------------------------ |
| NOV4e | ------------------------------------------------------------ |
| NOV4g | ------------------------------------------------------------ |
| NOV4h | ------------------------------------------------------------ |
| NOV4i | ------------------------------------------------------------ |
| NOV4j | ------------------------------------------------------------ |
| NOV4k | ------------------------------------------------------------ |
| NOV4l | ------------------------------------------------------------ |
| NOV4m | ------------------------------------------------------------ |
| NOV4n | ------------------------------------------------------------ |
| NOV4o | ------------------------------------------------------------ |
| NOV4a | CTAV |
| NOV4b | ---- |
| NOV4d | ---- |
| NOV4e | ---- |
| NOV4f | ---- |
| NOV4g | ---- |
| NOV4h | ---- |
| NOV4i | ---- |
| NOV4j | ---- |
| NOV4k | ---- |
| NOV4l | ---- |
| NOV4m | ---- |
| NOV4n | ---- |
| NOV4o | ---- |
| NOV4a | (SEQ ID NO: 32) |
| NOV4b | (SEQ ID NO: 34) |
| NOV4c | (SEQ ID NO: 36) |
| NOV4d | (SEQ ID NO: 38) |
| NOV4e | (SEQ ID NO: 40) |
| NOV4f | (SEQ ID NO: 42) |
| NOV4g | (SEQ ID NO: 44) |
| NOV4h | (SEQ ID NO: 46) |
| NOV4i | (SEQ ID NO: 48) |
| NOV4j | (SEQ ID NO: 50) |
| NOV4k | (SEQ ID NO: 52) |
| NOV4l | (SEQ ID NO: 54) |
| NOV4m | (SEQ ID NO: 56) |
| NOV4n | (SEQ ID NO: 58) |
| NOV4o | (SEQ ID NO: 60) |

Further analysis of the NOV4a protein yielded the following properties shown in Table 4C.

TABLE 4C
Protein Sequence Properties NOV4a

| | |
|---|---|
| SignalP analysis: | No Known Signal Sequence Predicted |
| PSORT II analysis: | |

| PSG: | a new signal peptide prediction method | |
|---|---|---|
| | N-region: | length 0; pos. chg 0; neg. chg 0 |
| | H-region: | length 15; peak value 1.77 |
| | PSG score: | −2.63 |

| | |
|---|---|
| GvH: | von Heijne's method for signal seq. recognition |
| | GvH score (threshold: −2.1): −8.90 |
| | possible cleavage site: between 50 and 51 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 3 | | |
| | Number of TMS(s) for threshold 0.5: 1 | | |
| | INTEGRAL | Likelihood = | Transmembrane 3472-3488 |
| | | −11.52 | |

| | |
|---|---|
| PERIPHERAL | Likelihood = 0.79 (at 1806) |

| | |
|---|---|
| | ALOM score: −11.52 (number of TMSs: 1) |
| MTOP: | Prediction of membrane topology (Hartmann et al.) |
| | Center position for calculation: 3479 |
| | Charge difference: 7.0 C(5.5)-N(−1.5) |
| | C > N: C-terminal side will be inside |

>>> Single TMS is located near the C-terminus
>>> membrane topology: type Nt (cytoplasmic tail 1 to 3471)

| MITDISC: | discrimination of mitochondrial targeting seq | | | |
|---|---|---|---|---|
| | R content: | 0 | Hyd Moment(75): | 6.28 |
| | Hyd Moment(95): | 5.99 | G content: | 3 |
| | D/E content: | 1 | S/T content: | 2 |

| | |
|---|---|
| | Score: −5.81 |
| Gavel: | prediction of cleavage sites for mitochondrial preseq |
| | cleavage site motif not found |

NUCDISC: discrimination of nuclear localization signals

| | |
|---|---|
| | pat4: KHRR (3) at 3492 |
| | pat4: HRRR (3) at 3493 |
| | pat4: RRRP (4) at 3494 |
| | pat4: RRPK (4) at 3495 |
| | pat7: PERGKRL (4) at 327 |
| | pat7: PSRGRRE (4) at 3135 |
| | bipartite: none |
| | content of basic residues: 6.9% |
| | NLS Score: 1.09 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: found
ILPN at 2237
RNA-binding motif: none
Actinin-type actin-binding motif:

type 1: none
type 2: none

NMYR: N-myristoylation pattern: GQNCTF
Prenylation motif: none
memYQRL: transport motif from cell surface to Golgi: none
Tyrosines in the tail: too long tail
Dileucine motif in the tail: found

LL at 474
LL at 818
LL at 956
LL at 1087
LL at 1141
LL at 1228
LL at 1316
LL at 1664
LL at 1854
LL at 2027
LL at 2164
LL at 2207
LL at 2298
LL at 2361
LL at 2807
LL at 2829
LL at 2988
LL at 3222
LL at 3390
LL at 3391

checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 70.6 |
| COIL: | Lupas's algorithm to detect coiled-coil regions |
| | total: 0 residues |

Final Results (k = 9/23):

```
34.8%: nuclear
26.1%: cytoplasmic
  8.7%: Golgi
  8.7%: mitochondrial
  8.7%: endoplasmic reticulum
  4.3%: vacuolar
  4.3%: vesicles of secretory system
  4.3%: peroxisomal
```

>> prediction for CG50377-04 is nuc (k = 23)

A search of the NOV4a protein against the Geneseq database, a proprietary database that contains sequences published in patents and patent publication, yielded several homologous proteins shown in Table 4D.

TABLE 4D
Geneseq Results for NOV4a

| | | NOV4a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABG79168 | Human cub and sushi domain | 283 . . . 3273 | 2943/3019 (97%) | 0.0 |
| | containing protein #1 - Homo sapiens, | 36 . . . 3052 | 2958/3019 (97%) | |
| | 3104 aa. [WO200264791-A2, 22-AUG- | | | |
| | 2002] | | | |
| ABG79169 | Human cub and sushi domain | 283 . . . 2829 | 2498/2634 (94%) | 0.0 |
| | containing protein #2 - Homo sapiens, | 36 . . . 2667 | 2512/2634 (94%) | |
| | 2669 aa. [WO200264791-A2, 22-AUG- | | | |
| | 2002] | | | |
| AAE20789 | Human C3b/C4b complement receptor | 437 . . . 3546 | 2010/3110 (64%) | 0.0 |
| | like protein #2 - Homo sapiens, 3100 | 1 . . . 3100 | 2479/3110 (79%) | |
| | aa. [WO200210199-A2, 07-FEB-2002] | | | |
| AAE20901 | Human C3b/C4b complement receptor | 437 . . . 3546 | 2009/3110 (64%) | 0.0 |
| | like protein #2, alternative version - | 1 . . . 3100 | 2478/3110 (79%) | |
| | Homo sapiens, 3100 aa. | | | |
| | [WO200210199-A2, 07-FEB-2002] | | | |
| AAE20788 | Rat C3b/C4b complement receptor like | 442 . . . 3546 | 1998/3105 (64%) | 0.0 |
| | protein - Rattus rattus, 3095 aa. | 1 . . . 3095 | 2474/3105 (79%) | |
| | [WO200210199-A2, 07-FEB-2002] | | | |

In a BLAST search of public sequence databases, the NOV4a protein was found to have homology to the proteins shown in the BLASTP data in Table 4E.

TABLE 4E
Public BLASTP Results for NOV4a

| | | NOV4a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q923L3 | CSMD1 - Mus musculus (Mouse), | 1 . . . 3546 | 2301/3546 (64%) | 0.0 |
| | 3564 aa. | 29 . . . 3564 | 2835/3546 (79%) | |
| AAK73475 | CUB and sushi multiple domains 1 | 1 . . . 3546 | 2298/3548 (64%) | 0.0 |
| | protein - Homo sapiens (Human), | 29 . . . 3566 | 2830/3548 (78%) | |
| | 3566 aa. | | | |
| CAD26764 | Sequence 6 from Patent | 437 . . . 3546 | 2010/3110 (64%) | 0.0 |
| | WO0210199 - Homo sapiens | 1 . . . 3100 | 2479/3110 (79%) | |
| | (Human), 3100 aa (fragment). | | | |
| CAD26836 | Sequence 3 from Patent | 442 . . . 3546 | 1998/3105 (64%) | 0.0 |
| | WO0210199 - Rattus rattus (Black | 1 . . . 3095 | 2474/3105 (79%) | |
| | rat), 3095 aa (fragment). | | | |
| CAD26763 | Sequence 1 from Patent | 469 . . . 3546 | 1987/3078 (64%) | 0.0 |
| | WO0210199 - Homo sapiens | 2 . . . 3069 | 2451/3078 (79%) | |
| | (Human), 3069 aa. | | | |

PFam analysis predicts that the NOV4a protein contains the domains shown in the Table 4F.

TABLE 4F
Domain Analysis of NOV4a

| | | Identities/ | |
|---|---|---|---|
| | | Similarities | |
| Pfam | NOV4a Match | for the | Expect |
| Domain | Region | Matched Region | Value |
| CUB | 4 . . . 109 | 39/116 (34%) | 2e−27 |
| | | 80/116 (69%) | |
| sushi | 117 . . . 174 | 22/65 (34%) | 4.7e−10 |
| | | 46/65 (71%) | |
| CUB | 180 . . . 281 | 45/116 (39%) | 5e−28 |
| | | 79/116 (68%) | |
| sushi | 321 . . . 378 | 24/65 (37%) | 3.7e−11 |
| | | 44/65 (68%) | |
| CUB | 383 . . . 491 | 43/118 (36%) | 1.7e−30 |
| | | 84/118 (71%) | |
| sushi | 499 . . . 552 | 19/63 (30%) | 4.9e−12 |
| | | 40/63 (63%) | |
| CUB | 554 . . . 661 | 43/116 (37%) | 2e−33 |
| | | 81/116 (70%) | |
| sushi | 669 . . . 726 | 16/65 (25%) | 5.7e−08 |
| | | 39/65 (60%) | |
| CUB | 730 . . . 835 | 46/116 (40%) | 1.2e−32 |
| | | 80/116 (69%) | |
| sushi | 845 . . . 898 | 17/63 (27%) | 1.8e−08 |
| | | 36/63 (57%) | |
| CUB | 902 . . . 1009 | 40/119 (34%) | 1.7e−19 |
| | | 79/119 (66%) | |
| sushi | 1017 . . . 1072 | 21/62 (34%) | 1.8e−10 |
| | | 44/62 (71%) | |
| CUB | 1076 . . . 1181 | 34/117 (29%) | 1.6e−24 |
| | | 81/117 (69%) | |
| sushi | 1189 . . . 1245 | 17/65 (26%) | 5.1e−10 |
| | | 45/65 (69%) | |
| CUB | 1249 . . . 1355 | 42/116 (36%) | 1.1e−21 |
| | | 73/116 (63%) | |
| sushi | 1363 . . . 1419 | 20/64 (31%) | 2.1e−06 |
| | | 40/64 (62%) | |
| CUB | 1423 . . . 1529 | 54/117 (46%) | 4.2e−39 |
| | | 89/117 (76%) | |
| sushi | 1537 . . . 1594 | 21/65 (32%) | 1.2e−09 |
| | | 42/65 (65%) | |
| CUB | 1598 . . . 1704 | 40/117 (34%) | 7.9e−15 |
| | | 70/117 (60%) | |
| sushi | 1715 . . . 1772 | 18/65 (28%) | 4.5e−06 |
| | | 40/65 (62%) | |
| CUB | 1776 . . . 1881 | 42/116 (36%) | 4e−31 |
| | | 82/116 (71%) | |
| sushi | 1889 . . . 1944 | 19/64 (30%) | 1.8e−10 |
| | | 43/64 (67%) | |
| CUB | 1948 . . . 2053 | 45/116 (39%) | 1.5e−29 |
| | | 77/116 (66%) | |
| sushi | 2061 . . . 2116 | 23/64 (36%) | 7.8e−13 |
| | | 47/64 (73%) | |
| CUB | 2120 . . . 2224 | 35/116 (30%) | 6.4e−24 |
| | | 75/116 (65%) | |
| sushi | 2232 . . . 2289 | 19/65 (29%) | 1.4e−09 |
| | | 46/65 (71%) | |
| CUB | 2293 . . . 2401 | 41/117 (35%) | 5.1e−20 |
| | | 79/117 (68%) | |
| sushi | 2406 . . . 2464 | 20/65 (31%) | 4.1e−08 |
| | | 39/65 (60%) | |
| sushi | 2469 . . . 2526 | 19/65 (29%) | 6.5e−09 |
| | | 43/65 (66%) | |
| sushi | 2531 . . . 2591 | 17/67 (25%) | 5.5e−09 |
| | | 43/67 (64%) | |
| sushi | 2596 . . . 2649 | 20/63 (32%) | 5.1e−13 |
| | | 41/63 (65%) | |
| sushi | 2654 . . . 2707 | 22/63 (35%) | 2.2e−13 |
| | | 40/63 (63%) | |
| sushi | 2712 . . . 2765 | 18/63 (29%) | 1.5e−13 |
| | | 41/63 (65%) | |
| sushi | 2770 . . . 2828 | 20/67 (30%) | 3.2e−14 |
| | | 47/67 (70%) | |
| sushi | 2833 . . . 2886 | 19/63 (30%) | 1.4e−09 |
| | | 39/63 (62%) | |
| sushi | 2894 . . . 2947 | 17/63 (27%) | 2.1e−13 |
| | | 40/63 (63%) | |
| sushi | 2952 . . . 3006 | 18/63 (29%) | 5.7e−08 |
| | | 40/63 (63%) | |
| sushi | 3011 . . . 3066 | 19/65 (29%) | 6.8e−09 |
| | | 42/65 (65%) | |
| sushi | 3071 . . . 3124 | 24/63 (38%) | 4.9e−14 |
| | | 46/63 (73%) | |
| sushi | 3129 . . . 3182 | 18/63 (29%) | 3.1e−11 |
| | | 42/63 (67%) | |
| sushi | 3190 . . . 3244 | 20/63 (32%) | 3.4e−09 |
| | | 45/63 (71%) | |
| sushi | 3249 . . . 3304 | 18/64 (28%) | 6.1e−10 |
| | | 39/64 (61%) | |

### Example 5

The NOV5 clone was analyzed, and the nucleotide and encoded polypeptide sequences are shown in Table 5A.

TABLE 5A
NOV5 Sequence Analysis

| | | |
|---|---|---|
| NOV5a, CG50389-04 | SEQ ID NO: 65 | 1897 bp |
| DNA Sequence | ORF Start: ATG at 51 | ORF Stop: TAA at 1785 |

GAATTCCGCCCGCCCACGGCGGCGGGGAAATACCTAGGCATGGAAGTGGCATGACAGGGCTCGTGTCC
CTGTCATATTTTCCACTCTCCACGAGGTCCTGCGCGCTTCAATCCTGCAGGCAGCCCGGTTTGGGGAT
GTGGTCCTTGCTGCTCTGCGGGTTGTCCATCGCCCTTCCACTGTCTGTCACAGCAGATGGATGCAAGG
ACATTTTTATGAAAAATGAGATACTTTCAGCAAGCCAGCCTTTTGCTTTTAATTGTACATTCCCTCCC
ATAACATCTGGGGAAGTCAGTGTAACATGGTATAAAAATTCTAGCAAAATCCCAGTGTCCAAAATCAT
ACAGTCTAGAATTCACCAGGACGAGACTTGGATTTTGTTTCTCCCCATGGAATGGGGGGACTCAGGAG
TCTACCAATGTGTTATAAAGGGTAGAGACAGCTGTCATAGAATACATGTAAACCTAACTGTTTTTGAA
AAACATTGGTGTGACACTTCCATAGGTGGTTTACCAAATTTATCAGATGAGTACAAGCAAATATTACA
TCTTGGAAAAGATGATAGTCTCACATGTCATCTGCACTTCCCGAAGAGTTGTGTTTTGGGTCCAATAA
AGTGGTATAAAGACTGTAACGAGATTAAAGGGGAGCGGTTCACTGTTTTGGAAACCAGGCTTTTGGTG
AGCAATGTCTCGGCAGAGGACAGAGGGAACTACGCGTGTCAAGCCATACTGACACACTCAGGGAAGCA
GTACGAGGTTTTAAATGGCATCACTGTGAGCATTAGTACCACTCTGATTGTGGACTGCAATGTAACAG
ACACCAAGGATAATACAAATCTACGATGCTGGAGAGTCAATAACACTTTGGTGGATGATTACTATGAT
GAATCCAAACGAATCAGAGAAGGGGTGGAAACCCATGTCTCTTTTCGGGAACATAATTTGTACACAGT
AAACATCACCTTCTTGGAAGTGAAAATGGAAGATTATGGCCTTCCTTTCATGTGCCACGCTGGAGTGT
CAACAGCATACATTATATTACAGCTCCCAGCTCCGGATTTTCGAGCTTACTTGATAGGAGGGCTTATC
GCCTTGGTGGCTGTGGCTGTGTCTGTTGTGTACATATACAACATTTTTAAGATCGACATTGTTCTTTG
GTATCGAAGTGCCTTCCATTCTACAGAGACCATAGTAGATGGGAAGCTGTATGACGCCTATGTCTTAT
ACCCCAAGCCCCACAAGGAAAGCCAGAGGCATGCCGTGGATGCCCTGGTGTTGAATATCCTGCCCGAG
GTGTTGGAGAGACAATGTGGATATAAGTTGTTTATATTCGGCAGAGATGAATTCCCTGGACAAGCCGT
GGCCAATGTCATCGATGAAAACGTTAAGCTGTGCAGGAGGCTGATTGTCATTGTGGTCCCCGAATCGC
TGGGCTTTGGCCTGTTGAAGAACCTGTCAGAAGAACAAATCGCGGTCTACAGTGCCCTGATCCAGGAC
GGGATGAAGGTTATTCTCGTTGAGCTGGAGAAAATCGAGGACTACACAGTCATGCCAGAGTCAATTCA
GTACATCAAACAGAAGCATGGTGCCATCCGGTGGCATGGGGACTTCACGGAGCAGTCACAGTGTATGA
AGACCAAGTTTTGGAAGACAGTGAGATACCACATGCCACCCAGAAGGTGTCGGCCGTTTCCTCCGGTC
CAGCTGCTGCAGCACACACCTTGCTGCCGCACCGCAGGCCCAGAACTAGGCTCAAGAAGAAAGAAGTG
TACTCTCACGACTGGCTAAGACTTGCTGGACTGACACCTATGGCTGGAAGATGACTTGTTTTGCTCCA
TGTCTCCTCATTCCTACACCTATTTTCTGCTGCAGGATGAGGCTAGGGTTAGCATTCTAGA

| | | | |
|---|---|---|---|
| NOV5a, CG50389-04 | SEQ ID NO: 66 | 578 aa | MW at 65576.2kD |
| Protein Sequence | | | |

MTGLVSLSYFPLSTRSCALQSCRQPGLGMWSLLLCGLSIALPLSVTADGCKDIFMKNEILSASQPFAF
NCTFPPITSGEVSVTWYKNSSKIPVSKIIQSRIHQDETWILFLPMEWGDSGVYQCVIKGRDSCHRIHV
NLTVFEKHWCDTSIGGLPNLSDEYKQILHLGKDDSLTCHLHFPKSCVLGPIKWYKDCNEIKGERFTVL
ETRLLVSNVSAEDRGNYACQAILTHSGKQYEVLNGITVSISTTLIVDCNVTDTKDNTNLRCWRVNNTL
VDDYYDESKRIREGVETHVSFREHNLYTVNITFLEVKMEDYGLPFMCHAGVSTAYIILQLPAPDFRAY
LIGGLIALVAVAVSVVYIYNIFKIDIVLWYRSAFHSTETIVDGKLYDAYVLYPKPHKESQRHAVDALV
LNILPEVLERQCGYKLFIFGRDEFPGQAVANVIDENVKLCRRLIVIVVPESLGFGLLKNLSEEQIAVY
SALIQDGMKVILVELEKIEDYTVMPESIQYIKQKHGAIRWHGDFTEQSQCMKTKFWKTVRYHMPPRRC
RPFPPVQLLQHTPCCRTAGPELGSRRKKCTLTTG

| | | |
|---|---|---|
| NOV5b, CG50389-06 | SEQ ID NO: 67 | 1802 bp |
| DNA Sequence | ORF Start: ATG at 471 | ORF Stop: TAA at 1743 |

GGGATGTGGTCCTTGCTGCTCTGCGGGTTGTCCATCGCCCTTCCACTGTCTGTCACAGCAGGTACGTT
CCGTGTGTCCTCCGTTCCCAGAGGCTGCCCGAGTCCACAGGCTCCTGGCCTGTCATTGGGAGCCCCCG
GGGATCGCGTCAGCCCGAGCGCGGCTCCTTCCCCTCCCCAGACCGCCAGGCGGAGTTCCGCGGAAGAG
GAAACAGAGAACCACCAGCTCCACGTGCTCGGATGCTCAGCCCTGAATGTCGCCTTTGAGCTTGGAGA
TGGATGCAAGGACATTTTTATGAAAAATGAGATACTTTCAGCAAGCCAGCCTTTTGCTTTTAATTGTA
CATTCCCTCCCATAACATCTGGGGAAGTCAGTGTAACATGGTATAAAAATTCTAGCAAAATCCCAGTG
TCCAAAATCATACAGTCTAGAATTCACCAGGACGAGACTTGGATTTTGTTTCTCCCCATGGAATGGGG
GGACTCAGGAGTCTACCAATGTGTTATAAAGACTGTAACGAGATTAAAGGGGAGCGGTTCACTGTTTT
GGAAACCAGGCTTTTGGTGAGCAATGTCTCGGCAGAGGACAGAGGGAACTACGCGTGTCAAGCCATAC
TGACACACTCAGGGAAGCAGTACGAGGTTTTAAATGGCATCACTGTGAGCATTACAGAAAGAGCTGGA
TATGGAGGAAGTGTCCCTAAAATCATTTATCCAAAAAATCATTCAATTGAAGTACAGCTTGGTACCAC
TCTGATTGTGGACTGCAATGTAACAGACACCAAGGATAATACAAATCTACGATGCTGGAGAGTCAATA
ACACTTTGGTGGATGATTACTATGATGAATCCAAACGAATCAGAGAAGGGGTGGAAACCCATGCCTCT
TTTCGGGAACATAATTTGTACACAGTAAACATCACCTTCTTGGAAGTGAAAATGGAAGATTATGGCCT
TCCTTTCGTGTGCCACGCTGGAGTGTCCACAGCATACATTATATTACAGCTCCCAGCTCCGGATTTTC
GAGCTTACTTGATAGGAGGGCTTATCGCCTTGGTGGCTGTGGCTGTGTCTGTTGTGTACATATACAAC
ATTTTTAAGATCGACATTGTTCTTTGGTACCGAAGTGCCTTCCATTCTACAGAGACCATAGTAGATGG
GAAGCTGTATGACGCCTATGTCTTATACCCCAAGCCCCACAAGGAAAGCCAGAGGCATGCCGTGGATG
CCCTGGTGTTGAATATCCTGCCCGAGGTGTTGGAGAGACAATGTGGATATAAGTTGTTTATATTCGGC
AGAGATGAATTCCTTGGACAAGCCGTGGCCAATGTCATCGATGAAAACGTTAAGCTGTGCAGGAGGCT
GATTGTCATTGTGGTCCCCGAATCGCTGGGCTTTGGCCTGTTGAAGAACCTGTCAGAAGAACAAATCG
CGGTCTACAGTGCCCTGATCCAGGACGGGATGAAGGTTATTCTCATTGAGCTGGAGAAAATCGAGGAC
TACACAGTCATGCCAGAGTCAATTCAGTACATCAAACAGAAGCATGGTGCCATCCGGTGGCATGGGGA
CTTCACGGAGCAGTCACAGTGTATGAAGACCAAGTTTTGGAAGACAGTGAGATACCACATGCCGCCCA
GAAGGTGTCGGCCGTTTCCTCCGGTCCAGCTGCCGCAGCACACACCTTGCTACCGCACCGCAGGCCCA
GAACTAGGCTCAAGAAGAAAGAAGTGTACTCTCACGACTGGCTAAGACTTGCTGGACTGACACCTATG
GCTGGAAGATGACTTGTTTTGCTCCATGTCTCCT

| | | | |
|---|---|---|---|
| NOV5b, CG50389-06 | SEQ ID NO: 68 | 424 aa | MW at 48256.3kD |
| Protein Sequence | | | |

MGGLRSLPMCYKDCNEIKGERFTVLETRLLVSNVSAEDRGNYACQAILTHSGKQYEVLNGITVSITER
AGYGGSVPKIIYPKNHSIEVQLGTTLIVDCNVTDTKDNTNLRCWRVNNTLVDDYYDESKRIREGVETH
ASFREHNLYTVNITFLEVKMEDYGLPFVCHAGVSTAYIILQLPAPDFRAYLIGGLIALVAVAVSVVYI
YNIFKIDIVLWYRSAFHSTETIVDGKLYDAYVLYPKPHKESQRHAVDALVLNILPEVLERQCGYKLFI
FGRDEFLGQAVANVIDENVKLCRPLIVIVVPESLGFGLLKNLSEEQIAVYSALIQDGMKVILIELEKI
EDYTVMPESIQYIKQKHGAIRWHGDFTEQSQCMKTKFWKTVRYHMPPRRCRPFPPVQLPQHTPCYRTA
GPELGSRRKKCTLTTG

| | | |
|---|---|---|
| NOV5c, 257448648 | SEQ ID NO: 69 | 891 bp |
| DNA Sequence | ORF Start: at 1 | ORF Stop: end of sequence |

GGATCCGATGGATGCAAGGACATTTTTATGAAAAATGAGATACTTTCAGCAAGCCAGCCTTTTGCTTT
TAATTGTACATTCCCTCCCATAACATCTGGGGAAGTCAGTGTAACATGGTATAAAAATTCTAGCAAAA
TCCCAGTGTCCAAAATCATACAGTCTAGAATTCACCAGGACGAGACTTGGATTTTGTTTCTCCCCATG
GAATGGGGGGACTCAGGAGTCTACCAATGTGTTATAAAGGGTAGAGACAGCTGTCATAGAATACATGT
AAACCTAACTGTTTTTGAAAAACATTGGTGTGACACTTCCATAGGTGGTTTACCAAATTTATCAGATG
AGTACAAGCAAATATTACATCTTGGAAAAGATGATAGTCTCACATGTCATCTGCACTTCCCGAAGAGT
TGTGTTTTGGGTCCAATAAAGTGGTATAAGGACTGTAACGAGATTAAAGGGGAGCGGTTCACTGTTTT
GGAAACCAGGCTTTTGGTGAGCAATGTCTCGGCAGAGGACAGAGGGAACTACGCGTGTCAAGCCATAC
TGACACACTCAGGGAAGCAGTACGAGGTTTTAAATGGCATCACTGTGAGCATTAGTACCACTCTGATT
GTGGACTGCAATGTAACAGACACCAAGGATAATACAAATCTACGATGCTGGAGAGTCAATAACACTTT
GGTGGATGATTACTATGATGAATCCAAACGAATCAGAGAAGGGGTGGAAACCCATGTCTCTTTTCGGG
AACATAATTTGTACACAGTAAACATCACCTTCTTGGAAGTGAAAATGGAAGATTATGGCCTTCCTTTC
ATGTGCCACGCTGGAGTGTCCACAGCATACATTATATTACAGCTCCCAGCTCCGGATTTTCGAGCTTA
CCTCGAG

| | | | |
|---|---|---|---|
| NOV5c, 257448648 | SEQ ID NO: 70 | 297 aa | MW at 33848.1kD |
| Protein Sequence | | | |

GSDGCKDIFMKNEILSASQPFAFNCTFPPITSGEVSVTWYKNSSKIPVSKIIQSRIHQDETWILFLPM
EWGDSGVYQCVIKGRDSCHRIHVNLTVFEKHWCDTSIGGLPNLSDEYKQILHLGKDDSLTCHLHFPKS
CVLGPIKWYKDCNEIKGERFTVLETRLLVSNVSAEDRGNYACQAILTHSGKQYEVLNGITVSISTTLI
VDCNVTDTKDNTNLRCWRVNNTLVDDYYDESKRIREGVETHVSFREHNLYTVNITFLEVKMEDYGLPF
MCHAGVSTAYIILQLPAPDFRAYLE

| | | |
|---|---|---|
| NOV5d, CG50389-01 | SEQ ID NO: 71 | 1769 bp |
| DNA Sequence | ORF Start: ATG at 45 | ORF Stop: TGA at 477 |

CGCCCGCCCACGGCGGCGGGGAAATACCTAGGCATGGAAGTGGCATGACAGGGCTCGTGTCCCTGTCA
TATTTTCCACTCTCCACGAGGTCCTGCGCGCTTCAATCCTGCAGGCAGCCCGGTTTGGGGATGTGGTC
CTTGCTGCTCTGCGGGTTGTCCATCGCCCTTCCACTGTCTGTCACAGCAGATGGATGCAAGGACATTT
TTATGAAAAATGAGATACTTTCAGCAAGCCAGCCTTTTGCTTTTAATTGTACATTCCCTCCCATAACA
TCTGGGGAAGTCAGTGTAACATGGTATAAAAATTCTAGCAAAATCCCAGTGTCCAAAATCATACAGTC
TAGAATTCACCAGGACGAGACTTGGATTTTGTTTCTCCCCATGGAATGGGGGGACTCAGGAGTCTACC
AATGTGTTATAAAGACTGTAACGAGATTAAAGGGGAGCGGTTCACTGTTTTGGAAACCAGGCTTTTGG
TGAGCAATGTCTCGGCAGAGGACAGAGGGAACTACGCGTGTCAAGCCATACTGACACACTCAGGGAAC
CAGTACGAGGTTTTAAATGGCATCACTGTGAGCATTACAGAAAGAGCTGGATATGGAGGAAGTGTCCC
TAAAATCATTTATCCAAAAAATCATTCAATTGAAGTACAGCTTGGTACCACTCTGATTGTGGACTGCA
ATGTAACAGACACCAAGGATAATACAAATCTACGATGCTGGAGAGTCAATAACACTTTGGTGGATGAT
TACTATGATGAATCCAAACGAATCAGAGAAGGGGTGGAAACCCATGTCTCTTTTCGGGAACATAATTT
GTACACAGTAAACATCACCTTCTTGGAAGTGAAAATGGAAGATTATGGCCTTCCTTTCATGTGCCACG
CTGGAGTGTCCACAGCATACATTATATTACAGCTCCCAGCTCCGGATTTTCGAGCTTACTTGATAGGA
GGGCTTATCGCCTTGGTGGCTGTGGCTGTGTCTGTTGTGTACATATACAACATTTTTAAGATCGACAT
TGTTCTTTGGTATCGAAGTGCCTTCCATTCTACAGAGACCATAGTAGATGGGAAGCTGTATGACGCCT
ATGTCTTATACCCCAAGCCCCACAAGGAAAGCCAGAGGCATGCCGTGGATGCCCTGGTGTTGAATATC
CTGCCCGAGGTGTTGGAGAGACAATGTGGATATAAGTTGTTTATATTCGGCAGAGATGAATTCCCTGC
ACAAGCCGTGGCCAATGTCATCGATGAAAACGTTAAGCTGTGCAGGAGGCTGATTGTCATTGTGGTCC
CCGAATCGCTGGGCTTTGGCCTGTTGAAGAACCTGTCAGAAGAACAAATCGCGGTCTACAGTGCCCTG
ATCCAGGACGGGATGAAGGTTATTCTCATTGAGCTGGAGAAAATCGAGGACTACACAGTCATGCCAGA
GTCAATTCAGTACATCAAACAGAAGCATGGTGCCATCCGGTGGCATGGGGACTTCACGGAGCAGTCAC
AGTGTATGAAGACCAAGTTTTGGAAGACAGTGAGATACCACATGCCGCCCAGAAGGTGTCGGCCGTTT
CTCCGGTCCACGTGCCGCAGCACACACCTCTGTACCGCACCGCAGGCCCAGAACTAGGCTCAAGAAGA
AAGAAGTGTACTCTCACGACTGGCTAAGACTTGCTGGACTGACACCTATGGCTGGAAGATGACTTGTT
TTGCTCCATGTCTCCTCATTCCTACACCTATTTTCTGCTGCAGGATGAGGCTAGGGTTAGCATTCTAC
A

| | | | |
|---|---|---|---|
| NOV5d, CG50389-01 | SEQ ID NO: 72 | 144 aa | MW at 15977.5kD |
| Protein Sequence | | | |

MTGLVSLSYFPLSTRSCALQSCRQPGLGMWSLLLCGLSIALPLSVTADGCKDIFMKNEILSASQPFAF
NCTFPPITSGEVSVTWYKNSSKIPVSKIIQSRIHQDETWILFLPMEWGDSGVYQCVIKTVTRLKGSGS
LFWKPGFW

| | | |
|---|---|---|
| NOV5e, CG50389-02 | SEQ ID NO: 73 | 1769 bp |
| DNA Sequence | ORF Start: ATG at 386 | ORF Stop: TAG at 1619 |

CGCCCGCCCACGGCGGCGGGGAAATACCTAGGCATGGAAGTGGCATGACAGGGCTCGTGTCCCTGTCA
TATTTTCCACTCTCCACGAGGTCCTGCGCGCTTCAATCCTGCAGGCAGCCCGGTTTGGGGATGTGGTC
CTTGCTGCTCTGCGGGTTGTCCATCGCCCTTCCACTGTCTGTCACAGCAGATGGATGCAAGGACATTT
TTATGAAAAATGAGATACTTTCAGCAAGCCAGCCTTTTGCTTTTAATTGTACATTCCCTCCCATAACA
TCTGGGGAAGTCAGTGTAACATGGTATAAAAATTCTAGCAAAATCCCAGTGTCCAAAATCATACAGTC
TAGAATTCACCAGGACGAGACTTGGATTTTGTTTCTCCCCATGGAATGGGGGGACTCAGGAGTCTACC
AATGTGTTATAAAGACTGTAACGAGATTAAAGGGGAGCGGTTCACTGTTTTGGAAACCAGGCTTTTGG
TGAGCAATGTCTCGGCAGAGGACAGAGGGAACTACGCGTGTCAAGCCATACTGACACACTCAGGGAAG
CAGTACGAGGTTTTAAATGGCATCACTGTGAGCATTACAGAAAGAGCTGGATATGGAGGAAGTGTCCC
TAAAATCATTTATCCAAAAAATCATTCAATTGAAGTACAGCTTGGTACCACTCTGATTGTGGACTGCA
ATGTAACAGACACCAAGGATAATACAAATCTACGATGCTGGAGAGTCAATAACACTTTGGTGGATGAT
TACTATGATGAATCCAAACGAATCAGAGAAGGGGTGGAAACCCATGTCTCTTTTCGGGAACATAATTT
GTACACAGTAAACATCACCTTCTTGGAAGTGAAAATGGAAGATTATGGCCTTCCTTTCATGTGCCACG
CTGGAGTGTCCACAGCATACATTATATTACAGCTCCCAGCTCCGGATTTTCGAGCTTACTTGATAGGA
GGGCTTATCGCCTTGGTGGCTGTGGCTGTGTCTGTTGTGTACATATACAACATTTTTAAGATCGACAT
TGTTCTTTGGTATCGAAGTGCCTTCCATTCTACAGAGACCATAGTAGATGGGAAGCTGTATGACGCCT
ATGTCTTATACCCCAAGCCCCACAAGGAAAGCCAGAGGCATGCCGTGGATGCCCTGGTGTTGAATATC
CTGCCCGAGGTGTTGGAGAGACAATGTGGATATAAGTTGTTTATATTCGGCAGAGATGAATTCCCTGG
ACAAGCCGTGGCCAATGTCATCGATGAAAACGTTAAGCTGTGCAGGAGGCTGATTGTCATTGTGGTCC
CCGAATCGCTGGGCTTTGGCCTGTTGAAGAACCTGTCAGAAGAACAAATCGCGGTCTACAGTGCCCTG
ATCCAGGACGGGATGAAGGTTATTCTCATTGAGCTGGAGAAAATCGAGGACTACACAGTCATGCCAGA
GTCAATTCAGTACATCAAACAGAAGCATGGTGCCATCCGGTGGCATGGGGACTTCACGGAGCAGTCAC
AGTGTATGAAGACCAAGTTTTGGAAGACAGTGAGATACCACATGCCGCCCAGAAGGTGTCGGCCGTTT
CTCCGGTCCACGTGCCGCAGCACACACCTCTGTACCGCACCGCAGGCCCAGAACTAGGCTCAAGAAGA
AAGAAGTGTACTCTCACGACTGGCTAAGACTTGCTGGACTGACACCTATGGCTGGAAGATGACTTGTT
TTGCTCCATGTCTCCTCATTCCTACACCTATTTTCTGCTGCAGGATGAGGCTAGGGTTAGCATTCTAG
A

| | | | |
|---|---|---|---|
| NOV5e, CG50389-02 | SEQ ID NO: 74 | 411 aa | MW at 46893.7kD |
| Protein Sequence | | | |

MGGLRSLPMCYKDCNEIKGERFTVLETRLLVSNVSAEDRGNYACQAILTHSGKQYEVLNGITVSITER
AGYGGSVPKIIYPKNHSIEVQLGTTLIVDCNVTDTKDNTNLRCWRVNNTLVDDYYDESKRIREGVETH
VSFREHNLYTVNITFLEVKMEDYGLPFMCHAGVSTAYIILQLPAPDFRAYLIGGLIALVAVAVSVVYI
YNIFKIDIVLWYRSAFHSTETIVDGKLYDAYVLYPKPHKESQRHAVDALVLNILPEVLERQCGYKLFI
FGRDEFPGQAVANVIDENVKLCRRLIVIVVPESLGFGLLKNLSEEQIAVYSALIQDGMKVILIELEKI
EDYTVMPESIQYIKQKHGAIRWHGDFTEQSQCMKTKFWKTVRYHMPPRRCRPFLRSTCRSTHLCTAPQ
AQN

| | | | |
|---|---|---|---|
| NOV5f, CG50389-03 | SEQ ID NO: 75 | 1827 bp | |
| DNA Sequence | ORF Start: ATG at 65 | ORF Stop: TAA at 1715 | J |

GTCATATTTTCCACTCTCCACGAGGTCCTGCGCGCTTCAATCCTGCAGGCAGCCCGGTTTGGGGATGT
GGTCCTTGCTGCTCTGCGGGTTGTCCATCGCCCTTCCACTGTCTGTCACAGCAGATGGATGCAAGGAC
ATTTTTATGAAAAATGAGATACTTTCAGCAAGCCAGCCTTTTGCTTTTAATTGTACATTCCCTCCCAT
AACATCTGGGGAAGTCAGTGTAACATGGTATAAAAATTCTAGCAAAATCCCAGTGTCCAAAATCATAC
AGTCTAGAATTCACCAGGACGAGACTTGGATTTTGTTTCTCCCCATGGAATGGGGGGACTCAGGAGTC
TACCAATGTGTTATAAAGGGTAGAGACAGCTGTCATAGAATACATGTAAACCTAACTGTTTTTGAAAA
ACATTGGTGTGACACTTCCATAGGTGGTTTACCAAATTTATCAGATGAGTACAAGCAAATATTACATC
TTGGAAAAGATGATAGTCTCACATGTCATCTGCACTTCCCGAAGAGTTGTGTTTTGGGTCCAATAAAG
TGGTATAAAGACTGTAACGAGATTAAAGGGGAGCGGTTCACTGTTTTGGAAACCAGGCTTTTGGTGAG
CAATGTCTCGGCAGAGGACAGAGGGAACTACGCGTGTCAAGCCATACTGACACACTCAGGGAAGCAGT
ACGAGGTTTTAAATGGCATCACTGTGAGCATTAGTACCACTCTGATTGTGGACTGCAATGTAACAGAC
ACCAAGGATAATACAAATCTACGATGCTGGAGAGTCAATAACACTTTGGTGGATGATTACTATGATGA
ATCCAAACGAATCAGAGAAGGGGTGGAAACCCATGTCTCTTTTCGGGAACATAATTTGTACACAGTAA
ACATCACCTTCTTGGAAGTGAAAATGGAAGATTATGGCCTTCCTTTCATGTGCCACGCTGGAGTGTCA
ACAGCATACATTATATTACAGCTCCCAGCTCCGGATTTTCGAGCTTACTTGATAGGAGGGCTTATCGC
CTTGGTGGCTGTGGCTGTGTCTGTTGTGTACATATACAACATTTTTAAGATCGACATTGTTCTTTGGT
ATCGAAGTGCCTTCCATTCTACAGAGACCATAGTAGATGGGAAGCTGTATGACGCCTATGTCTTATAC
CCCAAGCCCCACAAGGAAAGCCAGAGGCATGCCGTGGATGCCCTGGTGTTGAATATCCTGCCCGAGGT
GTTGGAGAGACAATGTGGATATAAGTTGTTTATATTCGGCAGAGATGAATTCCCTGGACAAGCCGTGG
CCAATGTCATCGATGAAAACGTTAAGCTGTGCAGGAGGCTGATTGTCATTGTGGTCCCCGAATCGCTG
GGCTTTGGCCTGTTGAAGAACCTGTCAGAAGAACAAATCGCGGTCTACAGTGCCCTGATCCAGGACGG
GATGAAGGTTATTCTCGTTGAGCTGGAGAAAATCGAGGACTACACAGTCATGCCAGAGTCAATTCAGT
ACATCAAACAGAAGCATGGTGCCATCCGGTGGCATGGGGACTTCACGGAGCAGTCACAGTGTATGAAG
ACCAAGTTTTGGAAGACAGTGAGATACCACATGCCACCCAGAAGGTGTCGGCCGTTTCCTCCGGTCCA
GCTGCTGCAGCACACACCTTGCTGCCGCACCGCAGGCCCAGAACTAGGCTCAAGAAGAAAGAAGTGTA
CTCTCACGACTGGCTAAGACTTGCTGGACTGACACCTATGGCTGGAAGATGACTTGTTTTGCTCCATG
TCTCCTCATTCCTACACCTATTTTCTGCTGCAGGATGAGGCTAGGGTTAGCATTCTAGA

| | | | |
|---|---|---|---|
| NOV5f, CG50389-03 | SEQ ID NO: 76 | 550 aa | MW at 62620.8kD |
| Protein Sequence | | | |

MWSLLLCGLSIALPLSVTADGCKDIFMKNEILSASQPFAFNCTFPPITSGEVSVTWYKNSSKIPVSKI
IQSRIHQDETWILFLPMEWGDSGVYQCVIKGRDSCHRIHVNLTVFEKHWCDTSIGGLPNLSDEYKQIL
HLGKDDSLTCHLHFPKSCVLGPIKWYKDCNEIKGERFTVLETRLLVSNVSAEDRGNYACQAILTHSGK
QYEVLNGITVSISTTLIVDCNVTDTKDNTNLRCWRVNNTLVDDYYDESKRIREGVETHVSFREHNLYT
VNITFLEVKMEDYGLPFMCHAGVSTAYIILQLPAPDFRAYLIGGLIALVAVAVSVVYIYNIFKIDIVL
WYRSAFHSTETIVDGKLYDAYVLYPKPHKESQRHAVDALVLNILPEVLERQCGYKLFIFGRDEFPGQA
VANVIDENVKLCRRLIVIVVPESLGFGLLKNLSEEQIAVYSALIQDGMKVILVELEKIEDYTVMPESI
QYIKQKHGAIRWHGDFTEQSQCMKTKFWKTVRYHMPPRRCRPFPPVQLLQHTPCCRTAGPELGSRRKK
CTLTTG

| | | |
|---|---|---|
| NOV5g, CG50389-05 | SEQ ID NO: 77 | 1829 bp |
| DNA Sequence | ORF Start: ATG at 42 | ORF Stop: TAA at 1767 |

GGTCCTGCGCGCTTCAATCCTGCAGGCAGCCCGGTTTGGGGATGTGGTCCTTGCAGCTCTGCGGGTTG
TCCATCGCCCTTCCACTGTCTGTCACAGCAGATGGATGCAAGGACATTTTTATGAAAAATGAGATACT
TTCAGCAAGCCAGCCTTTTGCTTTTAATTGTACATTCCCTCCCATAACATCTGGGGAGGTCAGTGTAA
CATGGTATAAAAATTCTAGCAAAATCCCAGTGTCCAAAATCATACAGTCTAGAATTCACCAGGACGAG
ACTTGGATTTTGTTTCTCCCCATGGAATGGGGGGACTCAGGAGTCTACCAATGTGTTATAAAGGGTAG
AGACAGCTGTCATAGAATACATGTAAACCTAACTGTTTTTGAAAAACATTGGTGTGACACTTCCATAG
GTGGTTTACCAAATTTATCAGATGAGTACAAGCAAATATTACATCTTGGAAAAGATGATAGTCTCACA
TGTCATCTGCACTTCCCGAAGAGTTGTGTTTTGGGTCCAATAAAGTGGTATAAGGACTGTAACGAGAT
TAAAGGGGAGCGGTTCACTGTTTTGGAAACCAGGCTTTTGGTGAGCAATGTCTCGGCAGAGGACAGAG
GGAACTACGCGTGTCAAGCCATACTGACACACTCAGGGAAGCAGTACGAGGTTTTAAATGGCATCACT
GTGAGCATTACAGAAAGAGCTGGATATGGAGGAAGTGTCCCTAAATCATTTATCCAAAAAAATCATTC
AATTGAAGTACAGCTTGGTACCACTCTGATTGTGGACTGCAATGTAACAGACACCAAGGATAATACAA
ATCTACGATGCTGGAGAGTCAATAACACTTTGGTGGATGATTACTATGATGAATCCAAACGAATCAGA
GAAGGGGTGGAAACCCATGTCTCTTTTCGGGAACATAATTTGTACACAGTAAACATCACCTTCTTGGA
AGTGAAAATGGAAGATTATGGCCTTCCTTTCATGTGCCACGCTGGAGTGTCCACAGCATACATTATAT
TACAGCTCCCAGCTCCGGATTTTCGAGCTTACTTGATAGGAGGGCTTATCGCCTTGGTGGCTGTGGCT
GTGTCTGTTGTGTACATATACAACATTTTTAAGATCGACATTGTTCTTTGGTACCGAAGTGCCTTCCA
TTCTACAGAGACCATAGTAGATGGGAAGCTGTATGACGCCTATGTCTTATACCCCAAGCCCCACAAGG
AAAGCCAGAGGCATGCCGTGGATGCCCTGGTGTTGAATATCCTGCCCGAGGTGTTGGAGAGACAATGT
GGATATAAGTTGTTTATATTCGGCAGAGATGAATTCCCTGGACAAGCCGTGGCCAATGTCATCGATGA
AAACGTTAAGCTGTGCAGGAGGCTGATTGTCATTGTGGTCCCCGAATCGCTGGGCTTTGGCCTGTTGA
AGAACCTGTCAGAAGAACAAATCGCGGTCTACAGTGCCCTGATCCAGGACGGGATGAAGGTTATTCTC
ATTGAGCTGGAGAAAATCGAGGACTACACAGTCATGCCAGAGTCAATTCAGTACATCAAACAGAAGCA
TGGTGCCATCCGGTGGCATGGGGACTTCACGGAGCAGTCACAGTGTATGAAGACCAAGTTTTGGAAGA
CAGTGAGATACCACATGCCGCCCAGAAGGTGTCGGCCGTTTCCTCCGGTCCAGCTGCCGCAGCACACA
CCTTGCTACCGCACCGCAGGCCCAGAACTAGGCTCAAGAAGAAAGAAGTGTACTCTCACGACTGGCTA
AGACTTGCTGGACTGACACCTATGGCTGGAAGATGACTTGTTTTGCTCCATGTCTCCTCAT

| | | | |
|---|---|---|---|
| NOV5g, CG50389-05 | SEQ ID NO: 78 | 575 aa | MW at 65402.8kD |
| Protein Sequence | | | |

MWSLQLCGLSIALPLSVTADGCKDIFMKNEILSASQPFAFNCTFPPITSGEVSVTWYKNSSKIPVSKI
IQSRIHQDETWILFLPMEWGDSGVYQCVIKGRDSCHRIHVNLTVFEKHWCDTSIGGLPNLSDEYKQIL
HLGKDDSLTCHLHFPKSCVLGPIKWYKDCNEIKGERFTVLETRLLVSNVSAEDRGNYACQAILTHSGK
QYEVLNGITVSITERAGYGGSVPKIIYPKNHSIEVQLGTTLIVDCNVTDTKDNTNLRCWRVNNTLVDD
YYDESKRIREGVETHVSFREHNLYTVNITFLEVKMEDYGLPFMCHAGVSTAYIILQLPAPDFRAYLIG
GLIALVAVAVSVVYIYNIFKIDIVLWYRSAFHSTETIVDGKLYDAYVLYPKPHKESQRHAVDALVLNI
LPEVLERQCGYKLFIFGRDEFPGQAVANVIDENVKLCRRLIVIVVPESLGFGLLKNLSEEQIAVYSAL
IQDGMKVILIELEKIEDYTVMPESIQYIKQKHGAIRWHGDFTEQSQCMKTKFWKTVRYHMPPRRCRPF
PPVQLPQHTPCYRTAGPELGSRRKKCTLTTG

| | | |
|---|---|---|
| NOV5h, SNP13382464 of | SEQ ID NO: 79 | 1897 bp |
| CG50389-04, DNA Sequence | ORF Start: ATG at 51 | ORF Stop: TAA at 1785 |
| | SNP Pos: 1160 | SNP Change: T to C |

GAATTCCGCCCGCCCACGGCGGCGGGGAAATACCTAGGCATGGAAGTGGCATGACAGGGCTCGTGTCC
CTGTCATATTTTCCACTCTCCACGAGGTCCTGCGCGCTTCAATCCTGCAGGCAGCCCGGTTTGGGGAT
GTGGTCCTTGCTGCTCTGCGGGTTGTCCATCGCCCTTCCACTGTCTGTCACAGCAGATGGATGCAAGG
ACATTTTTATGAAAAATGAGATACTTTCAGCAAGCCAGCCTTTTGCTTTTAATTGTACATTCCCTCCC
ATAACATCTGGGGAAGTCAGTGTAACATGGTATAAAAATTCTAGCAAAATCCCAGTGTCCAAAATCAT
ACAGTCTAGAATTCACCAGGACGAGACTTGGATTTTGTTTCTCCCCATGGAJJGGGGGGACTCAGGAG
TCTACCAATGTGTTATAAAGGGTAGAGACAGCTGTCATAGAATACATGTAAACCTAACTGTTTTTGAA
AAACATTGGTGTGACACTTCCATAGGTGGTTTACCAAATTTATCAGATGAGTACAAGCAAATATTACA
TCTTGGAAAAGATGATAGTCTCACATGTCATCTGCACTTCCCGAAGAGTTGTGTTTTGGGTCCAATAA
AGTGGTATAAAGACTGTAACGAGATTAAAGGGGAGCGGTTCACTGTTTTGGAAACCAGGCTTTTGGTG
AGCAATGTCTCGGCAGAGGACAGAGGGAACTACGCGTGTCAAGCCATACTGACACACTCAGGGAAGCA
GTACGAGGTTTTAAATGGCATCACTGTGAGCATTAGTACCACTCTGATTGTGGACTGCAATGTAACAG
ACACCAAGGATAATACAAATCTACGATGCTGGAGAGTCAATAACACTTTGGTGGATGATTACTATGAT
GAATCCAAACGAATCAGAGAAGGGGTGGAAACCCATGTCTCTTTTCGGGAACATAATTTGTACACAGT
AAACATCACCTTCTTGGAAGTGAAAATGGAAGATTATGGCCTTCCTTTCATGTGCCACGCTGGAGTGT
CAACAGCATACATTATATTACAGCTCCCAGCTCCGGATTTTCGAGCTTACTTGATAGGAGGGCTTATC
GCCTTGGTGGCTGTGGCTGTGTCTGTTGTGTACATATACAACATTTTTAAGATCGACATTGTTCTTTG
GTACCGAAGTGCCTTCCATTCTACAGAGACCATAGTAGATGGGAAGCTGTATGACGCCTATGTCTTAT
ACCCCAAGCCCCACAAGGAAAGCCAGAGGCATGCCGTGGATGCCCTGGTGTTGAATATCCTGCCCGAG
GTGTTGGAGAGACAATGTGGATATAAGTTGTTTATATTCGGCAGAGATGAATTCCCTGGACAAGCCGT
GGCCAATGTCATCGATGAAAACGTTAAGCTGTGCAGGAGGCTGATTGTCATTGTGGTCCCCGAATCGC
TGGGCTTTGGCCTGTTGAAGAACCTGTCAGAAGAACAAATCGCGGTCTACAGTGCCCTGATCCAGGAC
GGGATGAAGGTTATTCTCGTTGAGCTGGAGAAAATCGAGGACTACACAGTCATGCCAGAGTCAATTCA
GTACATCAAACAGAAGCATGGTGCCATCCGGTGGCATGGGGACTTCACGGAGCAGTCACAGTGTATGA
AGACCAAGTTTTGGAAGACAGTGAGATACCACATGCCACCCAGAAGGTGTCGGCCGTTTCCTCCGGTC
CAGCTGCTGCAGCACACACCTTGCTGCCGCACCGCAGGCCCAGAACTAGGCTCAAGAAGAAAGAAGTG
TACTCTCACGACTGGCTAAGACTTGCTGGACTGACACCTATGGCTGGAAGATGACTTGTTTTGCTCCA
TGTCTCCTCATTCCTACACCTATTTTCTGCTGCAGGATGAGGCTAGGGTTAGCATTCTAGA

| | | | |
|---|---|---|---|
| NOV5h, SNP13382464 of | SEQ ID NO: 80 | 1578 aa | MW at 65576.2kD |
| CG50389-04, Protein | SNP Pos: 370 | | SNP Change: Tyr to Tyr |
| Sequence | | | |

MTGLVSLSYFPLSTRSCALQSCRQPGLGMWSLLLCGLSIALPLSVTADGCKDIFMKNEILSASQPFAF
NCTFPPITSGEVSVTWYKNSSKIPVSKIIQSRIHQDETWILFLPMEWGDSGVYQCVIKGRDSCHRIHV
NLTVFEKHWCDTSIGGLPNLSDEYKQILHLGKDDSLTCHLHFPKSCVLGPIKWYKDCNEIKGERFTVL
ETRLLVSNVSAEDRGNYACQATLTHSGKQYEVLNGITVSISTTLIVDCNVTDTKDNTNLRCWRVNNTL
VDDYYDESKRIREGVETHVSFREHNLYTVNITFLEVKMEDYGLPFMCHAGVSTAYIILQLPAPDFPAY
LIGGLIALVAVAVSVVYIYNIFKIDIVLWYRSAFHSTETIVDGKLYDAYVLYPKPHKESQRHAVDALV
LNILPEVLERQCGYKLFIFGRDEFPGQAVANVIDENVKLCRRLIVIVVPESLGFGLLKNLSEEQIAVY
SALIQDGMKSIILVELEKIEDYTVMPESIQYIKQKHGAIRWHGDFTEQSQCMKTKFWKTVRYHMPPRR
RPFPPVQLLQHTPCCRTAGPELGSRRKKCTLTTG

A ClustalW comparison of the above protein sequences yields the following sequence alignment shown in Table 5B.

TABLE 5B
Comparison of the NOV5 protein sequences.

NOV5a ------------------------------------------------------------
NOV5b ------------------------------------------------------------
NOV5c -----------------------------------------------GSDGCKDIFMKNE
NOV5d ------------------------------------------------------------
NOV5e ------------------------------------------------------------
NOV5f ------------------------------MWSLLLCGLSIALPLSVTADGCKDIFMKNE
NOV5g MWSLQLCGLSIALPLSVTADGCKDIFMKNEILSASQPFAFNCTFPPITSGEVSVTWYKNS
NOV5a ------------------------------------------------------------
NOV5b ------------------------------------------------------------
NOV5c ILSASQPFAFNCTFPPITSGEVSVTWYKNSSKIPVSKIIQSRIHQDETWILFLPMEWGDS
NOV5d ------------------------------------------------------------
NOV5e ------------------------------------------------------------
NOV5f ILSASQPFAFNCTFPPITSGEVSVTWYKNSSKIPVSKIIQSRIHQDETWILFLPMEWGDS
NOV5g SKIPVSKIIQSRIHQDETWILFLPMEWGDSGVYQCVIKGRDSCHRIHVNLTVFEKHWCDT
NOV5a -------------------------------MTGLVSLS---------------------
NOV5b -------------------------------MGGLRSLP---------------------
NOV5c GVYQCVIKGRDSCHRIHVNLTVFEKHWCDTSIGGLPNLSDEYKQILHLGKDDSLTCHLHF
NOV5d -------------------------------MTGLVSLS---------------------
NOV5e -------------------------------MGGLRSLP---------------------
NOV5f GVYQCVIKGRDSCHRIHVNLTVFEKHWCDTSIGGLPNLSDEYKQILHLGKDDSLTCHLHF
NOV5g SIGGLPNLSDEYKQILHLGKDDSLTCHLHFPKSCVLGPI---------------------
NOV5a ---------YFPLSTRSCALQ--SCRQPGLGMWSLLLCGLSIALPLSVTADGCKDIFMKN
NOV5b ---------MCYKDCNEIKGERFTVLETRLLVSNVSAEDRGNYACQAILTHSGKQYEVLN
NOV5c PKSCVLGPIKWYKDCNEIKGERFTVLETRLLVSNVSAEDRGNYACQAILTHSGKQYEVLN
NOV5d ---------YFPLSTRSCALQ--SCRQPGLGMWSLLLCGLSIALPLSVTADGCKDIFMKN
NOV5e ---------MCYKDCNEIKGERFTVLETRLLVSNVSAEDRGNYACQAILTHSGKQYEVLN
NOV5f PKSCVLGPIKWYKDCNEIKGERFTVLETRLLVSNVSAEDRGNYACQAILTHSGKQYEVLN
NOV5g ---------KWYKDCNEIKGERFTVLETRLLVSNVSAEDRGNYACQAILTHSGKQYEVLN
NOV5a EILSASQP--------------------------FAFNCTFPPITSGEVSVTWYKNSSKI
NOV5b GITVSITERAGYGGSVPKIIYPKNHSIEVQLGTTLIVDCNVTDTKDNTNLRCWRVNNTLV
NOV5c GITVSIST-------------------------TLIVDCNVTDTKDNTNLRCWRVNNTLV
NOV5d EILSASQP--------------------------FAFNCTFPPITSGEVSVTWYKNSSKI
N0V5e GITVSITERAGYGGSVPKIIYPKNHSIEVQLGTTLIVDCNVTDTKDNTNLRCWRVNNTLV
NOV5f GITVSIST-------------------------TLIVDCNVTDTKDNTNLRCWRVNNTLV
NOV5g GITVSITEPAGYGGSVPKIIYPKNHSIEVQLGTTLIVDCNVTDTKDNTNLRCWRVNNTLV
NOV5a PVSKIIQSRIHQ---------DE---T-WILFLPMEWGDSGVYQCVIKGRDSCHRIHVNL
NOV5b DDYYDESKRIREGVETHASFREHNLYTVNITFLEVKMEDYGLPFVCHAGVSTAYIILQLP
NOV5c DDYYDESKRIREGVETHVSFREHNLYTVNITFLEVKMEDYGLPFMCHAGVSTAYIILQLP
NOV5d PVSKIIQSRIHQ---------DE---T-WILFLPMEWGDSGVYQCVIK---TVTRLKGSG
NOV5e DDYYDESKRIREGVETHVSFREHNLYTVNITFLEVKMEDYGLPFMCHAGVSTAYIILQLP
NOV5f DDYYDESKRIREGVETHVSFREHNLYTVNITFLEVKMEDYGLPFMCHAGVSTAYIILQLP
NOV5g DDYYDESKRIREGVETHVSFREHNLYTVNITFLEVKMEDYGLPFMCHAGVSTAYIILQLP
NOV5a TVFEKHWCDTSIGGLPNLSDEYKQILHLGKDDSLTCHLHFPKSCVLGPIKWYKDCNEIKG
NOV5b APDFRAYLIGGLIALVAVAVSVVYIYNIFKIDIVLWYRSAFHSTETIVDGKLYDAYVLYP
NOV5c APDFRAYLE---------------------------------------------------
NOV5d SLFWKPGFW---------------------------------------------------
NOV5e APDFRAYLIGGLIALVAVAVSVVYIYNIFKIDIVLWYRSAFHSTETIVDGKLYDAYVLYP
NOV5f APDFRAYLIGGLIALVAVAVSVVYIYNIFKIDIVLWYRSAFHSTETIVDGKLYDAYVLYP
NOV5g APDFRAYLIGGLIALVAVAVSVVYIYNIFKIDIVLWYRSAFHSTETIVDGKLYDAYVLYP
NOV5a ERFTVLETRLLVSNVSAEDRGNYACQAILTHSGKQYEVLNGITVSISTTLIVDCNVTDTK
NOV5b KPHKESQRHAVDALVLNILPEVLERQCGYKLFIFGRDEFLGQAVANVIDENVKLCRRLIV
NOV5c ------------------------------------------------------------
NOV5d ------------------------------------------------------------
NOV5e KPHKESQRHAVDALVLNILPEVLERQCGYKLFIFGRDEFPGQAVANVIDENVKLCRRLIV
NOV5f KPHKESQRHAVDALVLNILPEVLERQCGYKLFIFGRDEFPGQAVANVIDENVKLCRRLIV
NOV5g KPHKESQRHAVDALVLNILPEVLERQCGYKLFIFGRDEFPGQAVANVIDENVKLCRRLIV
NOV5a DNTNLRCWRVNNTLVDDYYDESKRIREGVETHVSFREHNLYTVNITFLEVKMEDYGLPFM
NOV5b IVVPESLGFGLLKNLSEEQIAVYSALIQDGMKVILIELEKIEDYTVMPESIQYIKQKHGA
NOV5c ------------------------------------------------------------
NOV5d ------------------------------------------------------------
NOV5e IVVPESLGFGLLKNLSEEQIAVYSALIQDGMKVILIELEKIEDYTVMPESIQYIKQKHGA
NOV5f IVVPESLGFGLLKNLSEEQIAVYSALIQDGMKVILVELEKIEDYTVMPESIQYIKQKHGA
NOV5g IVVPESLGFGLLKNLSEEQIAVYSALIQDGMKVILIELEKIEDYTVMPESIQYIKQKHGA
NOV5a CHAGVSTAYIILQLPAPDFRAYLIGGLIALVAVAVSVVYIYNIFKIDIVLWYRSAFHSTE
NOV5b IRWHGDFTEQSQCMKTKFWKTVRYHMPPRRCRPFPPVQLPQHTPCYRTAGPELGSRRKKC
NOV5c ------------------------------------------------------------
NOV5d ------------------------------------------------------------
NOV5e IRWHGDFTEQSQCMKTKFWKTVRYHMPPRRCRPFLRSTCRSTHLCTAPQAQN--------
N0V5f IRWHGDFTEQSQCMKTKFWKTVRYHMPPRRCRPFPPVQLLQHTPCCRTAGPELGSRRKKC
NOV5g IRWHGDFTEQSQCMKTKFWKTVRYHMPPRRCRPFPPVQLPQHTPCYRTAGPELGSRRKKC
NOV5a TIVDGKLYDAYVLYPKPHKESQRHAVDALVLNILPEVLERQCGYKLFIFGRDEFPGQAVA
NOV5b TLTTG-------------------------------------------------------
NOV5c ------------------------------------------------------------
NOV5d ------------------------------------------------------------
NOV5e ------------------------------------------------------------
NOV5f TLTTG-------------------------------------------------------
NOV5g TLTTG-------------------------------------------------------
NOV5a NVIDENVKLCRRLIVIVVPESLGFGLLKNLSEEQIAVYSALIQDGMKVILVELEKIEDYT
NOV5b ------------------------------------------------------------
NOV5c ------------------------------------------------------------
NOV5d ------------------------------------------------------------
NOV5e ------------------------------------------------------------
NOV5f ------------------------------------------------------------
NOV5g ------------------------------------------------------------
NOV5a VMPESIQYIKQKHGAIRWHGDFTEQSQCMKTKFWKTVRYHMPPRRCRPFPPVQLLQHTPC
NOV5b ------------------------------------------------------------
NOV5c ------------------------------------------------------------
NOV5d ------------------------------------------------------------
NOV5e ------------------------------------------------------------
NOV5f ------------------------------------------------------------
NOV5g ------------------------------------------------------------
NOV5a CRTAGPELGSRRKKCTLTTG
NOV5b --------------------
NOV5c --------------------
NOV5d --------------------
NOV5e --------------------
NOV5f --------------------
NOV5g --------------------
NOV5a (SEQ ID NO: 66)
NOV5b (SEQ ID NO: 68)
NOV5c (SEQ ID NO: 70)
NOV5d (SEQ ID NO: 72)
NOV5e (SEQ ID NO: 74)
NOV5f (SEQ ID NO: 76)
NOV5g (SEQ ID NO: 78)

Further analysis of the NOV5a protein yielded the following properties shown in Table 5C.

TABLE 5C
Protein Sequence Properties NOV5a

| | |
|---|---|
| SignalP analysis: | Cleavage site between residues 48 and 49 |
| PSORT II analysis: | |

| PSG: | a new signal peptide prediction method | |
|---|---|---|
| | N-region: | length 0; pos. chg 0; neg. chg 0 |
| | H-region: | length 14; peak value 7.84 |
| | PSG score: | 3.44 |

| | |
|---|---|
| GvH: | von Heijne's method for signal seq. recognition |
| | GvH score (threshold: −2.1): 0.58 |
| | possible cleavage site: between 40 and 41 |

>>> Seems to have a cleavable signal peptide (1 to 40)

| ALOM: | Klein et al's method for TM region allocation | | |
|---|---|---|---|
| | Init position for calculation: 41 | | |
| | Tentative number of TMS(s) for the threshold 0.5: 1 | | |
| | Number of TMS(s) for threshold 0.5: 1 | | |
| | INTEGRAL | Likelihood = | Transmembrane 342-358 |
| | | −9.39 | |

| | |
|---|---|
| PERIPHERAL | Likelihood = 0.58 (at 473) |

| | |
|---|---|
| | ALOM score: −9.39 (number of TMSs: 1) |
| MTOP: | Prediction of membrane topology (Hartmann et al.) |
| | Center position for calculation: 20 |
| | Charge difference: −1.0 C(1.0)-N(2.0) |
| | N >= C: N-terminal side will be inside |

>>> membrane topology: type 1a (cytoplasmic tail 359 to 578)

| MITDISC: | discrimination of mitochondrial targeting seq | | | |
|---|---|---|---|---|
| | R content: | 2 | Hyd Moment(75): | 1.16 |
| | Hyd Moment(95): | 1.99 | G content: | 4 |
| | D/E content: | 1 | S/T content: | 11 |

| | |
|---|---|
| | Score: −2.92 |
| Gavel: | prediction of cleavage sites for mitochondrial preseq |
| | R-2 motif at 33 CRQ\|PG |

NUCDISC: discrimination of nuclear localization signals

| | |
|---|---|
| | pat4: RRKK (5) at 569 |
| | pat7: PPRRCRP (4) at 540 |
| | pat7: PRRCRPF (5) at 541 |
| | bipartite: none |
| | content of basic residues: 10.6% |
| | NLS Score: 0.62 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: found

LL at 464
LL at 552

checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| | total: 0 residues |

Final Results (k = 9/23):

55.6%: endoplasmic reticulum
22.2%: Golgi
11.1%: plasma membrane
11.1%: extracellular, including cell wall

>> prediction for CG50389-04 is end (k = 9)

A search of the NOV5a protein against the Geneseq database, a proprietary database that contains sequences published in patents and patent publication, yielded several homologous proteins shown in Table 5D.

TABLE 5D
Geneseq Results for NOV5a

| | | NOV5a | Identities/ | | |
|---|---|---|---|---|---|
| | | Residues/ | Similarities for | | |
| Geneseq | Protein/Organism/Length [Patent #, | Match | the Matched | | Expect |
| Identifier | Date] | Residues | Region | | Value |
| ABG69605 | Human NOV6c protein - Homo | 1 . . . 578 | 578/578 | (100%) | 0.0 |
| | sapiens, 578 aa. [WO200250277-A2, | 1 . . . 578 | 578/578 | (100%) | |
| | 27-JUN-2002] | | | | |
| ABG69604 | Human NOV6b protein - Homo | 29 . . . 578 | 550/550 | (100%) | 0.0 |
| | sapiens, 550 aa. [WO200250277-A2, | 1 . . . 550 | 550/550 | (100%) | |
| | 27-JUN-2002] | | | | |
| AAE16577 | Human interleukin-1 receptor DNAX | 29 . . . 547 | 517/544 | (95%) | 0.0 |
| | designation 6 (IL-1RD6) protein - Homo | 1 . . . 544 | 519/544 | (95%) | |
| | sapiens, 562 aa. [US6326472-B1, 04- | | | | |
| | DEC-2001] | | | | |
| AAE03538 | Human Interleukin-1 Type 3 receptor | 29 . . . 547 | 517/544 | (95%) | 0.0 |
| | (IL-1-3R) - Homo sapiens, 562 aa. | 1 . . . 544 | 519/544 | (95%) | |
| | [US6239268-B1, 29-MAY-2001] | | | | |
| AAE06664 | Human interleukin-1R related protein 2 | 29 . . . 547 | 517/544 | (95%) | 0.0 |
| | (IL-1R6) - Homo sapiens, 562 aa. | 1 . . . 544 | 519/544 | (95%) | |
| | [WO200157219-A2, 09-AUG-2001] | | | | |

In a BLAST search of public sequence databases, the NOV5a protein was found to have homology to the proteins shown in the BLASTP data in Table 5E.

TABLE 5E
Public BLASTP Results for NOV5a

| | | NOV5a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9HB29 | Interleukin 1 receptor-like 2 precursor | 29 . . . 578 | 549/575 (95%) | 0.0 |
| | (IL-1Rrp2) (Interleukin-1 receptor | 1 . . . 575 | 550/575 (95%) | |
| | related protein 2) (IL1R-rp2) - Homo | | | |
| | sapiens (Human), 575 aa. | | | |
| CAC60374 | Sequence 12 from Patent WO0157219 | 29 . . . 547 | 517/544 (95%) | 0.0 |
| | - unidentified, 562 aa. | 1 . . . 544 | 519/544 (95%) | |
| Q62929 | Interleukin 1 receptor-like 2 precursor | 27 . . . 557 | 358/557 (64%) | 0.0 |
| | (Interleukin-1 receptor- related protein | 1 . . . 557 | 429/557 (76%) | |
| | 2) (IL1R-rp2) - Rattus norvegicus (Rat), | | | |
| | 561 aa. | | | |
| Q9ERS7 | Interleukin 1 receptor-like 2 precursor | 27 . . . 578 | 364/578 (62%) | 0.0 |
| | (IL-1Rrp2) - Mus musculus (Mouse), | 1 . . . 570 | 432/578 (73%) | |
| | 574 aa. | | | |
| P14778 | Interleukin-1 receptor, type I precursor | 33 . . . 553 | 224/560 (40%) | e−101 |
| | (IL-1R-1) (IL-1R-alpha) (P80) (Antigen | 7 . . . 552 | 314/560 (56%) | |
| | CD121a) - Homo sapiens (Human), 569 | | | |
| | aa. | | | |

PFam analysis predicts that the NOV5a protein contains the domains shown in the Table 5F.

TABLE 5F
Domain Analysis of NOV5a

| | | Identities/ | | |
|---|---|---|---|---|
| | | Similarities | | |
| Pfam | NOV5a Match | for the | | Expect |
| Domain | Region | Matched Region | | Value |
| ig | 63 . . . 125 | 11/64 | (17%) | 0.015 |
| | | 47/64 | (73%) | |
| ig | 167 . . . 225 | 13/60 | (22%) | 0.9 |
| | | 41/60 | (68%) | |
| TIR | 388 . . . 538 | 46/153 | (30%) | 2.8e−52 |
| | | 136/153 | (89%) | |

### Example 6.

The NOV6 clone was analyzed, and the nucleotide and encoded polypeptide sequences are shown in Table 6A.

TABLE 6A
NOV6 Sequence Analysis

| | | |
|---|---|---|
| NOV6a, CG50391-08 | SEQ ID NO: 81 | 2268 bp |
| DNA Sequence | ORF Start: ATG at 206 | ORF Stop: TGA at 1565 |

CTCTGACTGCTCCTATTGAGCTGTCTGCTCGCTGTGCCCGCTGTGCCTGCTGTGCCCGCGCTGTCGCC
GCTGCTACCGCGTCTACTGGACGCGGGAGACGCCAGCGAGCTGGTGATTGGAGCCCTGCGGAGAGCTC
AAGCGCCCAGCTCTGCCCGAGGAGCCCAGGCTGCCCCGTGAGTCCCATAGTTGCTGCAGGAGTGGAGC
CATGAGCTGCGTCCTGGGTGGTGTCATCCCCTTGGGGCTGCTGTTCCTGGTCTGCGGATCCCAAGGCT
ACCTCCTGCCCAACGTCACTCTCTTAGAGGAGCTGCTCAGCAAJJACCAGCACAACGAGTCTCACTCC
CGGGTCCGCAGAGCCATCCCCAGGGAGGACAAGGAGGAGATCCTCATGCTGCACAACAAGCTTCGGGG
CCAGGTGCAGCCTCAGGCCTCCAACATGGAGTACATGACCTGGGATGACGAACTGGAGAAGTCTGCTG
CAGCGTGGGCCAGTCAGTGCATCTGGGAGCACGGGCCCACCAGTCTGCTGGTGTCCATCGGGCAGAAC
CTGGGCGCTCACTGGGGCAGAATAGTTTGGGTCACCACCAACAAGATCGGTTGTGCTGTGAACACCTG
CCGGAAGATGACTGTCTGGGGAGAAGTTTGGGAGAACGCGGTCTACTTTGTCTGCAATTATTCTCCAA
AGGGGAACTGGATTGGAGAAGCCCCCTACAAGAATGGCCGGCCCTGCTCTGAGTGCCCACCCAGCTAT
GGAGGCAGCTGCAGGAACAACTTGTGTTACCGAGAAGAAACCTACACTCCAAAACCTGAAACGGACGA
GATGAATGAGGTGGAAACGGCTCCCATTCCTGAAGAAAACCATGTTTGGCTCCAACCGAGGGTGATGA
GACCCACCAAGCCCAAGAAAACCTCTGCGGTCAACTACATGACCCAAGTCGTCAGATGTGACACCAAG
ATGAAGGACAGGTGCAAAGGGTCCACGTGTAACAGGTACCAGTGCCCAGCAGGCTGCCTGAACCACAA
GGCGAAGATCTTTGGAAGTCTGTTCTATGAAAGCTCGTCTAGCATATGCCGCGCCGCCATCCACTACG
GGATCCTGGATGACAAGGGAGGCCTGGTGGATATCACCAGGAACGGGAAGGTCCCCTTCTTCGTGAAG
TCTGAGAGACACGGCGTGCAGTCCCTCAGCAAATACAAACCTTCCAGCTCATTCATGGTGTCAAAAGT
GAAAGTGCAGGATTTGGACTGCTACACGACCGTTGCTCAGCTGTGCCCGTTTGAAAAGCCAGCAACTC
ACTGCCCAAGAATCCATTGTCCGGCACACTGCAAAGACGAACCTTCCTACTGGGCTCCGGTGTTTGGA
ACCAACATCTATGCAGATACCTCAAGCATCTGCAAGACAGCCGTGCACGCGGGACTCATCAGCAACGA
GAGTGGGGGTGACGTGGACGTGATGCCCGTGGATAAAAAGAAGACCTACGTGGGCTCGCTCAGGAATG
GAGTTCAGTCTGAAAGCCTGGGGACTCCTCGGGATGGAAAGGCCTTCCGGATCTTTGCTGTCAGGCAG
TGAATTTCCAGCACCAGGGGAGAAGGGGCGTCTTCAGGAGGGCTTCGGGGTTTTGCTTTTATTTTTAT
TTTGTCATTGCGGGGTATATGGAGAGTCAGGAAACTTCCTTTGACTGATGTTCAGTGTCCATCACTTT
GTGGCCTGTGGGTGAGGTGACATCTCATCCCCTCACTGAAGCAACAGCATCCCAAGGTGCTCAGCCGG
ACTCCCTGGTGCCTGATCCTGCTGGGGCCCGGGGGTCTCCATCTGGACGTCCTCTCTCCTTTAGAGAT
CTGAGCTGTCTCTTAAAGGGGACAGTTGCCCAAAATGTTCCTTGCTATGTGTTCTTCTGTTGGTGGAG
GAAGTTGATTTCAACCTCCCTGCCAAAAGAACAAACCATTTGAAGCTCACAATTGTGAAGCATTCACG
GCGTCGGAAGAGGCCTTTTGAGCAAGCGCCAATGAGTTTCAGGAATGAAGTAGAAGGTAGTTATTTAA
AAATAAAAAACACAGTCCGTCCCTACCAATAGAGGAAAATGGTTTTAATGTTTGCTGGTCAGACAGAC
AAATGGGCTAGAGTAAGAGGGCTGCGGGTATGAGAGACCCCGGCTCCGCCCTGGCACGTGTCCTTGCT
GGCGGCCCGCCACAGGCCCCCTTCAATGGCCGCATTCAGGATGGCTCTATACACAGCAGTGCTGGTTT
ATGTAAAGTTCAGCAGTCACTTCA

| | | | |
|---|---|---|---|
| NOV6a, CG50391-08 | SEQ ID NO: 82 | 453 aa | MW at 50641.5kD |
| Protein Sequence | | | |

MSCVLGGVIPLGLLFLVCGSQGYLLPNVTLLEELLSKYQHNESHSRVRPAIPREDKEEILMLHNKLRG
QVQPQASNMEYMTWDDELEKSAAAWASQCIWEHGPTSLLVSIGQNLGAHWGRIVWVTTNKIGCAVNTC
RKMTVWGEVWENAVYFVCNYSPKGNWIGEAPYKNGRPCSECPPSYGGSCRNNLCYREETYTPKPETDE
MNEVETAPIPEENHVWLQPRVMRPTKPKKTSAVNYMTQVVRCDTKMKDRCKGSTCNRYQCPAGCLNHK
AKIFGSLFYESSSSICRAAIHYGILDDKGGLVDITRNGKVPFFVKSERHGVQSLSKYKPSSSFMVSKV
KVQDLDCYTTVAQLCPFEKPATHCPRIHCPAHCKDEPSYWAPVFGTNIYADTSSICKTAVHAGVISNE
SGGDVDVMPVDKKKTYVGSLRNGVQSESLGTPRDGKAFRIFAVRQ

| | | |
|---|---|---|
| NOV6b, CG50391-09 | SEQ ID NO: 83 | 2280 bp |
| DNA Sequence | ORF Start: ATG at 206 | ORF Stop: TGA at 1577 |

CTCTGACTGCTCCTATTGAGCTGTCTGCTCGCTGTGCCCGCTGTGCCTGCTGTGCCCGCGCTGTCGCC
GCTGCTACCGCGTCTACTGGACGCGGGAGACGCCAGCGAGCTGGTGATTGGAGCCCTGCGGAGAGCTC
AAGCGCCCAGCTCTGCCCGAGGACCCCAGGCTGCCCCGTGAGTCCCATAGTTGCTGCAGGAGTGGAGC
CATGAGCTGCGTCCTGGGTGGTGTCATCCCCTTGGGGCTGCTGTTCCTGGTCTGCGGATCCCAAGGCT
ACCTCCTGCCCAACGTCACTCTCTTAGAGGAGCTGCTCAGCAAATACCAGCACAACGAGTCTCACTCC
CGGGTCCGCAGAGCCATCCCCAGGGAGGACAAGGAGGAGATCCTCATGCTGCACAACAAGCTTCGGGG
CCAGGTGCAGCCTCAGGCCTCCAACATGGAGTACATGTATCGCTCTCCGGGGTTCCATGTGCAGTCCT
GGTATGACGAGGTGAAGGACTACACCTACCCCTACCCGAGCGAGTGCAACCCCTGGTGTCCAGAGAGG
TGCTCGGGGCCTATGTGCACGCACTACACACAGATAGTTTGGGCCACCACCAACAAGATCGGTTGTGC
TGTGAACACCTGCCGGAAGATGACTGTCTGGGGAGAAGTTTGGGAGAACGCGGTCTACTTTGTCTGCA
ATTATTCTCCAAAGGGGAACTGGATTGGAGAAGCCCCCTACAAGAATGGCCGGCCCTGCTCTGAGTGC
CCACCCAGCTATGGAGGCAGCTGCAGGAACAACTTGTGTTACCGAGAAGAAACCTACACTCCAAAACC
TGAAACGGACGAGATGAATGAGGTGGAAACGGCTCCCATTCCTGAAGAAAACCATGTTTGGCTCCAAC
CGAGGGTGATGAGACCCACCAAGCCCAAGAAAACCTCTGCGGTCAACTACATGACCCAAGTCGTCAGA
TGTGACACCAAGATGAAGGACAGGTGCAAAGGGTCCACGTGTAACAGGTACCAGTGCCCAGCAGGCTG
CCTGAACCACAAGGCGAAGATCTTTGGAACTCTGTTCTATGAAAGCTCGTCTAGCATATGCCGCGCCG
CCATCCACTACGGGATCCTGGATGACAAGGGAGGCCTGGTGGATATCACCAGGAACGGGAAGGTCCCC
TTCTTCGTGAAGTCTGAGAGACACGGCGTGCAGTCCCTCAGCAAATACAAACCTTCCAGCTCATTCAT
GGTGTCAAAAGTGAAAGTGCAGGATTTGGACTGCTACACGACCGTTGCTCAGCTGTGCCCGTTTGAAA
AGCCAGCAACTCACTGCCCAAGAATCCATTGTCCGGCACACTGCAAAGACGAACCTTCCTACTGGGCT
CCGGTGTTTGGAACCAACATCTATGCAGATACCTCAAGCATCTGCAAGACAGCCGTGCACGCGGGAGT
CATCAGCAACGAGAGTGGGGGTGACGTGGACGTGATGCCCGTGGATAAAAAGAAGACCTACGTGGGCT
CGCTCAGGAATGGAGTTCAGTCTGAAAGCCTGGGGACTCCTCGGGATGGAAAGGCCTTCCGGATCTTT
GCTGTCAGGCAGTGAATTTCCACCACCAGGGGAGAAGGGGCGTCTTCAGGAGGGCTTCGGGGTTTTGC
TTTTATTTTTATTTTGTCATTGCGGGGTATATGGAGAGTCAGGAAACTTCCTTTGACTGATGTTCAGT
GTCCATCACTTTGTGGCCTGTGGGTGAGGTGACATCTCATCCCCTCACTGAAGCAACAGCATCCCAAG
GTGCTCAGCCGGACTCCCTGGTGCCTGATCCTGCTGGGGCCCGGGGGTCTCCATCTGGACGTCCTCTC
TCCTTTAGAGATCTGAGCTGTCTCTTAAAGGGGACAGTTGCCCAAAATGTTCCTTGCTATGTGTTCTT
CTGTTGGTGGAGGAAGTTGATTTCAACCTCCCTGCCAAAAGAACAAACCATTTGAAGCTCACAATTGT
GAAGCATTCACGGCGTCGGAAGAGGCCTTTTGAGCAAGCGCCAATGAGTTTCAGGAATGAAGTAGAAG
GTAGTTATTTAAAAATAAAAAACACAGTCCGTCCCTACCAATAGAGGAAAATGGTTTTAATGTTTGCT
GGTCAGACAGACAAATGGGCTAGAGTAAGAGGGCTGCGGGTATGAGAGACCCCGGCTCCGCCCTGGCA
CGTGTCCTTGCTGGCGGCCCGCCACAGGCCCCCTTCAATGGCCGCATTCAGGATGGCTCTATACACAG
CAGTGCTGGTTTATGTAAAGTTCAGCAGTCACTTCA

| | | | |
|---|---|---|---|
| NOV6b, CG50391-09 | SEQ ID NO: 84 | 457 aa | MW at 51515.4kD |
| Protein Sequence | | | |

MSCVLGGVIPLGLLFLVCGSQGYLLPNVTLLEELLSKYQHNESHSRVRRAIPREDKEEILMLHNKLRG
QVQPQASNMEYMYRSPGFHVQSWYDEVKDYTYPYPSECNPWCPERCSGPMCTHYTQIVWATTNKIGCA
VNTCRKMTVWGEVWENAVYFVCNYSPKGNWIGEAPYKNGRPCSECPPSYGGSCRNNLCYREETYTPKP
ETDEMNEVETAPIPEENHVWLQPRVMRPTKPKKTSAVNYMTQVvRCDTKNKDRCKGSTCNRYQCPAGC
LNHKAKIFGTLFYESSSSICRAAIHYGILDDKGGLVDITRNGKVPFFVKSERHGVQSLSKYKPSSSFM
VSKVKVQDLDCYTTVAQLCPFEKPATHCPRIHCPAHCKDEPSYWAPVFGTNIYADTSSICKTAVHAGV
ISNESGGDVDVMPVDKKKTYVGSLRNGVQSESLGTPRDGKAFRIFAVRQ

| | | |
|---|---|---|
| NOV6c, CG50391-10 | SEQ ID NO: 85 | 2106 bp |
| DNA Sequence | ORF Start: ATG at 206 | ORF Stop: TGA at 1403 |

CTCTGACTGCTCCTATTGAGCTGTCTGCTCGCTGTGCCCGCTGTGCCTGCTGTGCCCGCGCTGTCGCC
GCTGCTACCGCGTCTACTGGACGCGGGAGACGCCAGCGAGCTGGTGATTGGAGCCCTGCGGAGAGCTC
AAGCGCCCAGCTCTGCCCGAGGAGCCCAGGCTGCCCCGTGAGTCCCATAGTTGCTGCAGGAGTGGAGC
CATGAGCTGCGTCCTGGGTGGTGTCATCCCCTTGGGGCTGCTGTTCCTGGTCTGCGGATCCCAAGGCT
ACCTCCTGCCCAACGTCACTCTCTTAGAGGAGCTGCTCAGCAAATACCAGCACAACGAGTCTCACTCC
CGGGTCCGCAGAGCCATCCCCAGGGAGGACAAGGAGGAGATCCTCATGCTGCACAACAAGCTTCGGGG
CCAGGTGCAGCCTCAGGCCTCCAACATGGAGTACATGACCTGGGATGACGAACTGGAGAAGTCTGCTG
CAGCGTGGGCCAGTCAGTGCATCTGGGAGCACGGGCCCACCAGTCTGCTGGTGTCCATCGGGCAGAAC
CTGGGCGCTCACTGGGGCAGGGGGAACTGGATTGGAGAAGCCCCCTACAAGAATGGCCGGCCCTGCTC
TGAGTGCCCACCCAGCTATGGAGGCAGCTGCAGGAACAACTTGTGTTACCGAGAAGAAACCTACACTC
CAAAACCTGAAACGGACGAGATGAATGAGGTGGAAACGGCTCCCATTCCTGAAGAAAACCATGTTTGG
CTCCAACCGAGGGTGATGAGACCCACCAAGCCCAAGAAAACCTCTGCGGTCAACTACATGACCCAAGT
CGTCAGATGTGACACCAAGATGAAGGACAGGTGCAAAGGGTCCACGTGTAACAGGTACCAGTGCCCAG
CAGGCTGCCTGAACCACAAGGCGAAGATCTTTGGAAGTCTGTTCTATGAAAGCTCGTCTAGCATATGC
CGCGCCGCCATCCACTACGGGATCCTGGATGACAAGGGAGGCCTGGTGGATATCACCAGGAACGGGAA
GGTCCCCTTCTTCGTGAAGTCTGAGAGACACGGCGTGCAGTCCCTCGTGCAGGATTTGGACTGCTACA
CGACCGTTGCTCAGCTGTGCCCGTTTGAAAAGCCAGCAACTCACTGCCCAAGAATCCATTGTCCGGCA
CACTGCAAAGACGAACCTTCCTACTGGGCTCCGGTGTTTGGAACCAACATCTATGCAGATACCTCAAG
CATCTGCAAGACAGCCGTGCACGCGGGAGTCATCAGCAACGAGAGTGGGGGTGACGTGGACGTGATGC
CCGTGGATAAAAAGAAGACCTACGTGGGCTCGCTCAGGAATGGAGTTCAGTCTGAAAGCCTGGGGACT
CCTCGGGATGGAAAGGCCTTCCGGATCTTTGCTGTCAGGCAGTGAATTTCCAGCACCAGGGGAGAAGG
GGCGTCTTCAGGAGGGCTTCGGGGTTTTGCTTTTATTTTTATTTTGTCATTGCGGGGTATATGGAGAG
TCAGGAAACTTCCTTTGACTGATGTTCAGTGTCCATCACTTTGTGGCCTGTGGGTGAGGTGACATCTC
ATCCCCTCACTGAAGCAACAGCATCCCAAGGTGCTCAGCCGGACTCCCTGGTGCCTGATCCTGCTGGG
GCCCGGGGGTCTCCATCTGGACGTCCTCTCTCCTTTAGAGATCTGAGCTGTCTCTTAAAGGGGACAGT
TGCCCAAAATGTTCCTTGCTATGTGTTCTTCTGTTGGTGGAGGAAGTTGATTTCAACCTCCCTGCCAA
AAGAACAAACCATTTGAAGCTCACAATTGTGAAGCATTCACGGCGTCGGAAGAGGCCTTTTGAGCAAC
CGCCAATGAGTTTCAGGAATGAAGTAGAAGGTAGTTATTTAAAAATAAAAAACACAGTCCGTCCCTAC
CAATAGAGGAAAATGGTTTTAATGTTTGCTGGTCAGACAGACAAATGGGCTAGAGTAAGAGGGCTGCG
GGTATGAGAGACCCCGGCTCCGCCCTGGCACGTGTCCTTGCTGGCGGCCCGCCACAGGCCCCCTTCAA
TGGCCGCATTCAGGATGGCTCTATACACAGCAGTGCTGGTTTATGTAAAGTTCAGCAGTCACTTCA

| | | | |
|---|---|---|---|
| NOV6c, CG50391-10 | SEQ ID NO: 86 | 399 aa | MW at 44463.2kD |
| Protein Sequence | | | |

MSCVLGGVIPLGLLFLVCGSQGYLLPNVTLLEELLSKYQHNESHSRVRPAIPREDKEEILMLHNKLRG
QVQPQASNMEYMTWDDELEKSAAAWASQCIWEHGPTSLLVSIGQNLGAHWGRGNWIGEAPYKNGRPCS
ECPPSYGGSCRNNLCYREETYTPKPETDEMNEVETAPIPEENHVWLQPRVMRPTKPKKTSAVNYMTQV
VRCDTKMKDRCKGSTCNRYQCPAGCLNHKAKIFGSLFYESSSSICRAAIHYGILDDKGGLVDITRNGK
VPFFVKSERHGVQSLVQDLDCYTTVAQLCPFEKPATHCPRIHCPAHCKDEPSYWAPVFGTNIYADTSS
ICKTAVHAGVISNESGGDVDVMPVDKKKTYVGSLRNGVQSESLGTPRDGKAFRIFAVRQ

| | | |
|---|---|---|
| NOV6d, CG50391-01 | SEQ ID NO: 87 | 2305 bp |
| DNA Sequence | ORF Start: ATG at 453 | ORF Stop: TGA at 1602 |

CTCTGACTGCTCCTATTGAGCTGTCTGCTCGCTGTGCCCGCTGTGCCTGCTGTGCCCGCGCTGTCGCC
GCTGCTACCGCGTCTACTGGACGCGGGAGACGCCAGCGAGCTGGTGATTGGAGCCCTGCGGAGAGCTC
AAGCGCCCAGCTCTGCCCGAGGAGCCCAGGCTGCCCCGTGAGTCCCATAGTTGCTACAGGAGTGGAGC
CATGAGCTGCGTCCTGGGTGGTGTCATCCCCTTGGGGCTGCTGTTCCTGGTCCGCGGATCCCAAGGCT
ACCTCCTGCCCAACGTCACTCTCTTAGAGGAGCTGCTCAGCAAATACCAGCACAACGAGTCTCACTCC
CGGGTCCGCAGAGCCATCCCCAGGGAGGACAAGGAGGAGATCCTCATGCTGCACAACAAGCTTCGGGG
CCAGGTGCAGCCTCAGGCCTCCAACATGGAGTACATGACCTGGGATGACGAACTGGGGCAGGTATCGC
TCTCCGGGGTTCCATGTGCAGTCCTGGTATGACGAGGTGAAGGACTACACCTACCCCTACCCGAGCGA
GTGCAACCCCTGGTGTCCAGAGAGGTGCTCGGGGCCTATGTGCACGCACTACACACAGATAGTTTGGG
CCACCACCAACAAGATCGGTTGTGCTGTGAACACCTGCCGGAAGATGACTGTCTGGGGAGAAGTTTGG
GAGAACGCGGTCTACTTTGTCTGCAATTATTCTCCAAAGGGGAACTGGATTGGAGAAGCCCCCTACAA
GAATGGCCGGCCCTGCTCTGAGTGCCCACCCAGCTATGGAGGCAGCTGCAGGAACAACTTGTGTTACC
GAGAAGAACCTACACTCCAACCTGAAACGGACGAGATGAATGAGGTGGTGGAAACGGCTCCCATTCCT
GAAGAAAACCATGTTTGGCTCCAACCGAGGGTGATGAGACCCACCAAGCCCAAGAAAACCTCTGCGGT
CAACTACATGACCCAAGTCGTCAGATGTGACACCAAGATGAAGGACAGGTGCAAAGGGTCCACGTGTA
ACAGGTACCAGTGCCCAGCAGGCTGCCTGACCACAAGGCGAGATCTTTGGAACTCTCTGTTCTATGAA
AGCTCGTCTAGCATATGCCGCGCCGCCATCCACTACGGGATCCTGGATGACAAGGGAGGCCTGGTGGA
TATCACCAGGAACGGGAAGGTCCCCTTCTTCGTGAAGTCTGAGAGACACGGCGTGCAGTCCCTCAGCA
AATACAAACCTTCCAGCTCATTCATGGTGTCAAAAGTGAAAGTGCAGGATTTGGACTGCTACACGACC
GTTGCTCAGCTGTGCCCGTTTGAAAAGCCAGCAACTCACTGCCCAAGAATCCATTGTCCGGCACACTG
CAAAGACGAACCTTCCTACTGGGCTCCGGTGTTTGGAACCAACATCTATGCAGATACCTCAAGCATCT
GCAAGACAGCCGTGCACGCGGGAGTCATCAGCAACGAGAGTGGGGGTGACGTGGACGTGATGCCCGTG
GATAAAAAGAAGACCTACGTGGGCTCGCTCAGGAATGGAGTTCAGTCTGAAAGCCTGGGGACTCCTCG
GGATGGAAAGGCCTTCCGGATCTTTGCTGTCAGGCAGTGAATTTCCAGCACCAGGGGAGAAGGGGCGT
CTTCAGGAGGGCTTCGGGGTTTTGCTTTTATTTTTATTTTGTCATTGCGGGGTATATGGAGAGTCAGG
AAACTTCCTTTGACTGATGTTCAGTGTCCATCACTTTGTGGCCTGTGGGTGAGGTGACATCTCATCCC
CTCACTGAAGCAACAGCATCCCAAGGTGCTCAGCCGGACTCCCTGGTGCCTGATCCTGCTGGGGCCCG
GGGGTCTCCATCTGGACGTCCTCTCTCCTTTAGAGATCTGAGCTGTCTCTTAAAGGGGACAGTTGCCC
AAAATGTTCCTTGCTATGTGTTCTTCTGTTGGTGGAGGAAGTTGATTTCAACCTCCCTGCCAAAAGAA
CAAACCATTTGAAGCTCACAATTGTGAAGCATTCACGGCGTCGGAAGAGGCCTTTTGAGCAAGCGCCA
ATGAGTTTCAGGAATGAAGTAGAAGGTAGTTATTTAAAAATAAAAAACACAGTCCGTCCCTACCAATA
GAGGAAAATGGTTTTAATGTTTGCTGGTCAGACAGACAAATGGGCTAGAGTAAGAGGGCTGCGGGTAT
GAGAGACCCCGGCTCCGCCCTGGCACGTGTCCTTGCTGGCGGCCCGCCACAGGCCCCCTTCAATGGCC
GCATTCAGGATGGCTCTATACACAGCAGTGCTGGTTTATGTAAAGTTCAGCAGTCACTTCA

| | | | |
|---|---|---|---|
| NOV6d, CG50391-01 | SEQ ID NO: 88 | 383 aa | MW at 43185.7kD |
| ProteinSequence | | | |

MTNWGRYRSPGFHVQSWYDEVKDYTYPYPSECNPWCPERCSGPMCTHYTQIVWATTNKIGCAVNTCRK
MTVWGEVWENAVYFVCNYSPKGNWIGEAPYKNGRPCSECPPSYGGSCRNNLCYREETYTPKPETDEMN
EVETAPIPEENHVWLQPRVMRPTKPKKTSAVNYMTQVVRCDTKMKDRCKGSTCNRYQCPAGCLNHKAK
IFGTLFYESSSSICRAAIHYGILDDKGGLVDITRNGKVPFFVKSERHGVQSLSKYKPSSSFMVSKVKV
QDLDCYTTVAQLCPFEKPATHCPRIHCPAHCKDEPSYWAPVFGTNIYADTSSICKTAVHAGVISNESG
GDVDVMPVDKKKTYVGSLRNGVQSESLGTPRDGKAFRIFAVRQ

| | | |
|---|---|---|
| NOV6e, CG50391-02 | SEQ ID NO: 89 | 1669 bp |
| DNA Sequence | ORF Start: ATG at 205 | ORF Stop: TAG at 1636 |

TCTGACTGCTCCTATTGAGCTGTCTGCTCGCTGTGCCCGCTGTGCCTGCTGTGCCCGCGCTGTCGCCC
CTGCTACCGCGTCTACTGGACGCGGGAGACGCCAGCGAGCTGGTGATTGGAGCCCTGCGGAGAGCTCA
AGCGCCCAGCTCTGCCCGAGGAGCCCAGGCTGCCCCGTGAGTCCCATAGTTGCTGCAGGAGTGGAGCC
ATGAGCTGCGTCCTGGGTGGTGTCATCCCCTTGGGGCTGCTGTTCCTGGTCTGCGGATCCCAAGGCTA
CCTCCTGCCCAACGTCACTCTCTTAGAGGAGCTGCTCAGCAAATACCAGCACAACGAGTCTCACTCCC
GGGTCCGCAGAGCCATCCCCAGGGAGGACAAGGAGGAGATCCTCATGCTGCACAACAAGCTTCGGGGC
CAGGTGCAGCCTCAGGCCTCCAACATGGAGTACATGACCTGGGATGACGAACTGGAGAAGTCTGCTGC
AGCGTGGGCCAGTCAGTGCATCTGGGAGCACGGGCCCACCGGTCTGCTGGTGTCCATCGGGCAGAACC
TGGGCGCTCACTGGGGCAGGTATCGCTCTCCGGGGTTCCATGTGCAGTCCTGGTATGACGAGGTGAAG
GACTACACCTACCCCTACCCGAGCGAGTGCAACCCCTGGTGTCCAGAGAGGTGCTCGGGGCCTATGTG
CACGCACTACACACAGATAGTTTGGGCCACCACCAACAAGATCGGTTGTGCTGTGAACACCTGCCGGA
AGATGACTGTCTGGGGAGAAGTTTGGGAGAACGCGGTCTACTTTGTCTGCAATTATTCTCCAAAGGGG
AACTGGATTGGAGAAGCCCCCTACAAGAATGGCCGGCCCTGCTCTCAGTGCCCACCCAGCTATGGAGG
CAGCTGCAGGAACAACTTGTGTTACCGAGAAGAAACCTACACTCCAAAACCTGAAACGGACGAGATGA
ATGAGGTGGAAACGGCTCCCATTCCTGAAGAAAACCATGTTTGGCTCCAACCGAGGGTGATGAGACCC
ACCAAGCCCAAGAAAACCTCTTCGGTCAACTACATGACCCAAGTCGTCTTATGTGACACCAAGATGAA
GGACAGGTGCAAAGGGTCCACGTGTAACAGGTACCAGTGCCCAGCAGGCTGCCTGAACCACAAGGCGA
AGATCTTTGGAACTCTGTTCTATGAAAGCTCGTCTAGCATATGCCGCGCCGCCATCCACTACGGGATC
CTGGATGACAAGGGAGGCCTGGTGGATATCACCAGGAACGGGAAGGTCCCCTTCTTCGTGAAGTCTGA
GAGACACGGCGTGCAGTCCCTCAGCAAATACAAACCTTCCAGCTCATTCATGGTGTCAAAAGTGAAAG
TGCAGGATTTGGACTGCTACACGACCGTTGCTCAGCTGTGCCCGTTTGAAAAGCCAGCAACTCACTGC
CCAAGAATCCATTGTCCGGCACACTGCAAAGACGAACCTTCCTACTGGGCTCCGGTGTTTGGAACCAA
CATCTATGCAGATACCTCAAGCATCTGCAAGACAGCCGTGCACGCGGGAGTCATCAGCAACGAGAGTG
GGGGTGACGTGGACGTGATGCCCGTGGATAAAAAGAAGACCTACACCTGCCCGGCAGCCGCTCGAGCC
CTATAGTGTAAACCGATTCGCAGCACACTGGCGCCGT

| | | | |
|---|---|---|---|
| NOV6e, CG50391-02 | SEQ ID NO: 90 | 477 aa | MW at 53587.7kD |
| Protein Sequence | | | |

MSCVLGGVIPLGLLFLVCGSQGYLLPNVTLLEELLSKYQHNESHSRVRRATPREDKEEILMLHNKLRG
QVQPQASNNEYMTWDDELEKSAAAWASQCIWEHGPTGLLVSIGQNLGAHWGRYRSPGFHVQSWYDEVK
DYTYPYPSECNPWCPERCSGPMCTHYTQIVWATTNKIGCAVNTCRKMTVWGEVWENAVYFVCNYSPKG
NWIGEAPYKNGRPCSQCPPSYGGSCRNNLCYREETYTPKPETDENNEVETAPIPEENHVWLQPRVMRP
TKPKKTSSVNYMTQVVLCDTKMKDRCKGSTCNRYQCPAGCLMIKAKIFGTLFYESSSSICRAAIHYGI
LDDKGGLVDITRNGKVPFFVKSERHGVQSLSKYKPSSSFMVSKVKVQDLDCYTTVAQLCPFEKPATHC
PRIHCPAHCKDEPSYWAPVFGTNTYADTSSICKTAVHAGVISNESGGDVDVMPVDKKKTYTCPAAARA
L

| | | |
|---|---|---|
| NOV6f, CG50391-03 | SEQ ID NO: 91 | 2400 bp |
| DNA Sequence | ORF Start: ATG at 206 | ORF Stop: TGA at 1697 |

CTCTGACTGCTCCTATTGAGCTGTCTGCTCGCTGTGCCCGCTGTGCCTGCTGTGCCCGCGCTGTCGCC
GCTGCTACCGCGTCTACTGGACGCGGGAGACGCCAGCGAGCTGGTGATTGGAGCCCTGCGGAGAGCTC
AAGCGCCCAGCTCTGCCCGAGGAGCCCAGGCTGCCCCGTGAGTCCCATAGTTGCTGCAGGAGTGGAGC
CATGAGCTGCGTCCTGGGTGGTGTCATCCCCTTGGGGCTGCTGTTCCTGGTCCGCGGATCCCAAGGCT
ACCTCCTGCCCAACGTCACTCTCTTAGAGGAGCTGCTCAGCAAATACCAGCACAACGAGTCTCACTCC
CGGGTCCGCAGAGCCATCCCCAGGGAGGACAAGGAGGAGATCCTCATGCTGCACAACAAGCTTCGGGG
CCAGGTGCAGCCTCAGGCCTCCAACATGGAGTACATGACCTGGGATGACGAACTGGAGAAGTCTGCTG
CAGCGTGGGCCAGTCAGTGCATCTGGGAGCACGGGCCCACCGGTCTGCTGGTGTCCATCGGGCAGAAC
CTGGGCGCTCACTGGGGCAGGTATCGCTCTCCGGGGTTCCATGTGCAGTCCTGGTATGACGAGGTGAA
GGACTACACCTACCCCTACCCGAGCGAGTGCAACCCCTGGTGTCCAGAGAGGTGCTCGGGGCCTATGT
GCACGCACTACACACAGATAGTTTGGGCCACCACCAACAAGATCGGTTGTGCTGTGAACACCTGCCGG
AAGATGACTGTCTGGGGAGAAGTTTGGGAGAACGCGGTCTACTTTGTCTGCAATTATTCTCCAAAGGG
GAACTGGATTGGAGAAGCCCCCTACAAGAATGGCCGGCCCTGCTCTGAGTGCCCACCCAGCTATGGAG
GCAGCTGCAGGAACAACTTGTGTTACCGAGAAGAAACCTACACTCCAAAACCTGAAACGGACGAGATG
AATGAGGTGGAAACGGCTCCCATTCCTGAAGAAAACCATGTTTGGCTCCAACCGAGGGTGATGAGACC
CACCAAGCCCAAGAAAACCTCTGCGGTCAACTACATGACCCAAGTCGTCAGATGTGACACCAAGATGA
AGGACAGGTGCAAAGGGTCCACGTGTAACAGGTACCAGTGCCCAGCAGGCTGCCTGAACCACAAGGCG
AAGATCTTTGGAAGTCTGTTCTATGAAAGCTCGTCTAGCATATGCCGCGCCGCCATCCACTACGGGAT
CCTGGATGACAAGGGAGGCCTGGTGGATATCACCAGGAACGGGAAGGTCCCCTTCTTCGTGAAGTCTG
AGAGACACGGCGTGCAGTCCCTCAGCAAATACAAACCTTCCAGCTCATTCATGGTGTCAAAAGTGAAA
GTGCAGGATTTGGACTGCTACACGACCGTTGCTCAGCTGTGCCCGTTTGAAAAGCCAGCAACTCACTG
CCCAAGAATCCATTGTCCGGCACACTGCAAAGACGAACCTTCCTACTGGGCTCCGGTGTTTGGAACCA
ACATCTATGCAGATACCTCAAGCATCTGCAAGACAGCCGTGCACGCGGGAGTCATCAGCAACGAGAGT
GGGGGTGACGTGGACGTGATGCCCGTGGATAAAAAGAAGACCTACGTGGGCTCGCTCAGGAATGGAGT
TCAGTCTGAAAGCCTGGGGACTCCTCGGGATGGAAAGGCCTTCCGGATCTTTGCTGTCAGGCAGTGAA
TTTCCAGCACCAGGGGAGAAGGGGCGTCTTCAGGAGGGCTTCGGGGTTTTGCTTTTATTTTTATTTTG
TCATTGCGGGGTATATGGAGAGTCAGGAAACTTCCTTTGACTGATGTTCAGTGTCCATCACTTTGTGG
CCTGTGGGTGAGGTGACATCTCATCCCCTCACTGAAGCAACAGCATCCCAAGGTGCTCAGCCGGACTC
CCTGGTGCCTGATCCTGCTGGGGCCCGGGGGTCTCCATCTGGACGTCCTCTCTCCTTTAGAGATCTGA
GCTGTCTCTTAAAGGGGACAGTTGCCCAAAATGTTCCTTGCTATGTGTTCTTCTGTTGGTGGAGGAAG
TTGATTTCAACCTCCCTGCCAAAAGAACAAACCATTTGAAGCTCACAATTGTGAAGCATTCACGGCGT
CGGAAGAGGCCTTTTGAGCAAGCGCCAATGAGTTTCAGGAATGAAGTAGAAGGTAGTTATTTAAAAAT
AAAAAACACAGTCCGTCCCTACCAATAGAGGAAAATGGTTTTAATGTTTGCTGGTCAGACAGACAAAT
GGGCTAGAGTAAGAGGGCTGCGGGTATGAGAGACCCCGGCTCCGCCCTGGCACGTGTCCTTGCTGGCG
GCCCGCCACAGGCCCCCTTCAATGGCCGCATTCAGGATGGCTCTATACACAGCAGTGCTGGTTTATGT
AAAGTTCAGCAGTCACTTCA

| | | | |
|---|---|---|---|
| NOV6f, CG50391-03 | SEQ ID NO: 92 | 497 aa | MW at 55928.2kD |
| Protein Sequence | | | |

MSCVLGGVIPLGLLFLVRGSQGYLLPNVTLLEELLSKYQHNESHSRVRRAIPREDKEEILMLHNKLRG
QVQPQASNMEYMTWDDELEKSAAAWASQCIWEHGPTGLLVSIGQNLGAHWGRYRSPGFHVQSWYDEVK
DYTYPYPSECNPWCPERCSGPMCTHYTQIVWATTNKIGCAVNTCRKMTVWGEVWENAVYFVCNYSPKG
NWIGEAPYKNGRPCSECPPSYGGSCRNNLCYREETYTPKPETDEMNEVETAPIPEENHVWLQPRVMRP
TKPKKTSAVNYMTQVVRCDTKMKDRCKGSTCNRYQCPAGCLNHKAKIFGSLFYESSSSICRAAIHYGI
LDDKGGLVDITPNGKVPFFVKSERHGVQSLSKYKPSSSFMVSKVKVQDLDCYTTVAQLCPFEKPATHC
PRIHCPAHCKDEPSYWAPVFGTNIYADTSSICKTAVHAGVISNESGGDVDVMPVDKKKTYVGSLRNGV
QSESLGTPRDGKAFRIFAVRQ

| | | |
|---|---|---|
| NOV6g, CG50391-04 | SEQ ID NO: 93 | 2403 bp |
| DNA Sequence | ORF Start: ATG at 206 | ORF Stop: TGA at 1700 |

CTCTGACTGCTCCTATTGAGCTGTCTGCTCGCTGTGCCCGCTGTGCCTGCTGTGCCCGCGCTGTCGCC
GCTGCTACCGCGTCTACTGGACGCGCGAGACGCCAGCGAGCTGGTGATTGGAGCCCTGCGGAGAGCTC
AAGCGCCCAGCTCTGCCCGAGGAGCCCAGGCTGCCCCGTGAGTCCCATAGTTGCTGCAGGAGTGGAGC
CATGAGCTGCGTCCTGGGTGGTGTCATCCCCTTGGGGCTGCTGTTCCTGGTCCGCGGATCCCAAGGCT
ACCTCCTGCCCAACGTCACTCTCTTAGAGGAGCTGCTCAGCAAATACCAGCACAACGAGTCTCACTCC
CGGGTCCGCAGAGCCATCCCCAGGGAGGACAAGGAGGAGATCCTCATGCTGCACAACAAGCTTCGGGG
CCAGGTGCAGCCTCAGGCCTCCAACATGGAGTACATGACCTGGGATGACGAACTGGAGAAGTCTGCTG
CAGCGTGGGCCAGTCAGTGCATCTGGGAGCACGGGCCCACCAGTCTGCTGGTGTCCATCGGGCAGAAC
CTGGGCGCTCACTGGGGCAGGAGGTATCGCTCTCCGGGGTTCCATGTGCAGTCCTGGTATGACGAGGT
GAAGGACTACACCTACCCCTACCCGAGCGAGTGCAACCCCTGGTGTCCAGAGAGGTGCTCGGGGCCTA
TGTGCACGCACTACACACAGATAGTTTGGGCCACCACCAACAAGATCGGTTGTGCTGTGAACACCTGC
CGGAAGATGACTGTCTGGGGAGAAGTTTGGGAGAACGCGGTCTACTTTGTCTGCAATTATTCTCCAAA
GGGGAACTGGATTGGAGAAGCCCCCTACAAGAATGGCCGGCCCTGCTCTGAGTGCCCACCCAGCTATG
GAGGCAGCTGCAGGAACAACTTGTGTTACCGAGAAGAAACCTACACTCCAAAACCTGAAACGGACGAG
ATGAATGAGGTGGAAACGGCTCCCATTCCTGAAGAAAACCATGTTTGGCTCCAACCGAGGGTGATGAG
ACCCACCAAGCCCAAGAAAACCTCTGCGGTCAACTACATGACCCAAGTCGTCAGATGTGACACCAAGA
TGAAGGACAGGTGCAAAGGGTCCACGTGTAACAGGTACCAGTGCCCAGCAGGCTGCCTGAACCACAAG
GCGAAGATCTTTGGAAGTCTGTTCTATGAAAGCTCGTCTAGCATATGCCGCGCCGCCATCCACTACGG
GATCCTGGATGACAAGGGAGGCCTGGTGGATATCACCAGGAACGGGAAGGTCCCCTTCTTCGTGAAGT
CTGAGAGACACGGCGTGCAGTCCCTCAGCAAATACAAACCTTCCAGCTCATTCATGGTGTCAAAAGTG
AAAGTGCAGGATTTGGACTGCTACACGACCGTTGCTCAGCTGTGCCCGTTTGAAAAGCCAGCAACTCA
CTGCCCAAGAATCCATTGTCCGGCACACTGCAAAGACGAACCTTCCTACTGGGCTCCGGTGTTTGGAA
CCAACATCTATGCAGATACCTCAAGCATCTGCAAGACAGCCGTGCACGCGGGAGTCATCAGCAACGAG
AGTGGGGGTGACGTGGACGTGATGCCCGTGGATAAAAAGAAGACCTACGTGGGCTCGCTCAGGAATGG
AGTTCAGTCTGAAAGCCTGGGGACTCCTCGGGATGGAAAGGCCTTCCGGATCTTTGCTGTCAGGCAGT
GAATTTCCAGCACCAGGGGAGAAGGGGCGTCTTCAGGAGGGCTTCGGGGTTTTGCTTTTATTTTTATT
TTGTCATTGCGGGGTATATGGAGAGTCAGGAAACTTCCTTTGACTGATGTTCAGTGTCCATCACTTTG
TGGCCTGTGGGTGAGGTGACATCTCATCCCCTCACTGAAGCAACAGCATCCCAAGGTGCTCAGCCGGA
CTCCCTGGTGCCTGATCCTGCTGGGGCCCGGGGGTCTCCATCTGGACGTCCTCTCTCCTTTAGAGATC
TGAGCTGTCTCTTAAAGGGGACAGTTGCCCAAAATGTTCCTTGCTATGTGTTCTTCTGTTGGTGGAGG
AAGTTGATTTCAACCTCCCTGCCAAAAGAACAAACCATTTGAAGCTCACAATTGTGAAGCATTCACGG
CGTCGGAAGAGGCCTTTTGAGCAAGCGCCAATGAGTTTCAGGAATGAAGTAGAAGGTAGTTATTTAAA
AATAAAAAACACAGTCCGTCCCTACCAATAGAGGAAAATGGTTTTAATGTTTGCTGGTCAGACAGACA
AATGGGCTAGAGTAAGAGGGCTGCGGGTATGAGAGACCCCGGCTCCGCCCTGGCACGTGTCCTTGCTG
GCGGCCCGCCACAGGCCCCCTTCAATGGCCGCATTCAGGATGGCTCTATACACAGCAGTGCTGGTTTA
TGTAAAGTTCAGCAGTCACTTCA

| | | | |
|---|---|---|---|
| NOV6g, CG50391-04 | SEQ ID NO: 94 | 498 aa | MW at 56114.4kD |
| Protein Sequence | | | |

MSCVLGGVIPLGLLFLVRGSQGYLLPNVTLLEELLSKYQHNESHSRVRRAIPREDKEEILMLHNKLRG
QVQPQASNMEYMTWDDELEKSAAAWASQCIWEHGPTSLLVSIGQNLGAHWGRRYRSPGFHVQSWYDEV
KDYTYPYPSECNPWCPERCSGPMCTHYTQIVWATTNKIGCAVNTCRKMTVWGEVWENAVYFVCNYSPK
GNWIGEAPYKNGRPCSECPPSYGGSCRNNLCYREETYTPKPETDEMNEVETAPIPEENHVWLQPRVMR
PTKPKKTSAVNYMTQVVRCDTKMKDRCKGSTCNRYQCPAGCLNHKAKIFGSLFYESSSSICRAAIHYG
ILDDKGGLVDITRNGKVPFFVKSERHGVQSLSKYKPSSSFMVSKVKVQDLDCYTTVAQLCPFEKPATH
CPRIHCPAHCKDEPSYWAPVFGTNIYADTSSICKTAVHAGVISNESGGDVDVMPVDKKKTYVGSLRNG
VQSESLGTPRDGKAFRIFAVRQ

| | | |
|---|---|---|
| NOV6h, CG50391-05 | SEQ ID NO: 95 | 2412 bp |
| DNA Sequence | ORF Start: ATG at 206 | ORF Stop: TGA at 1709 |

CTCTGACTGCTCCTATTGAGCTGTCTGCTCGCTGTGCCCGCTGTGCCTGCTGTGCCCGCGCTGTCGCC
GCTGCTACCGCGTCTACTGGACGCGGGAGACGCCAGCGAGCTGGTGATTGGAGCCCTGCGGAGAGCTC
AAGCGCCCAGCTCTGCCCGAGGAGCCCAGGCTGCCCCGTGAGTCCCATAGTTGCTGCAGGAGTGGAGC
CATGAGCTGCGTCCTGGGTGGTGTCATCCCCTTGGGGCTGCTGTTCCTGGTCCGCGGATCCCAAGGCT
ACCTCCTGCCCAACGTCACTCTCTTAGAGGAGCTGCTCAGCAAATACCAGCACAACGAGTCTCACTCC
CGGGTCCGCAGAGCCATCCCCAGGGAGGACAAGGAGGAGATCCTCATGCTGCACAACAAGCTTCGGGG
CCAGGTGCAGCCTCAGGCCTCCAACATGGAGTACATGACCTGGGATGACGAACTGGAGAAGTCTGCTG
CAGCGTGGGCCAGTCAGTGCATCTGGGAGCACGGGCCCACCGGTCTGCTGGTGTCCATCGGGCAGAAC
CTGGGCGCTCACTGGGGCAGGTATCGCTCTCCGGGGTTCCATGTGCAGTCCTGGTATGACGAGGTGAA
GGACTACACCTACCCCTACCCGAGCGAGTGCAACCCCTGGTGTCCAGAGAGGTGCTCGGGGCCCATGT
GCACGCACTACACACAGGTAACTCAGATAGTTTGGGCCACCACCAACAAGATCGGTTGTGCTGTGAAC
ACCTGCCGGAAGATGACTGTCTGGGGAGAAGTTTGGGAGAACGCGGTCTACTTTGTCTGCAATTATTC
TCCAAAGAGGGGGAACTGGATTGGAGAAGCCCCCTACAAGAATGGCCGGCCCTGCTCTGAGTGCCCAC
CCAGCTATGGAGGCAGCTGCAGGAACAACTTGTGTTACCGAGAAGAAACCTACACTCCAAAACCTGAA
ACGGACGAGATGAATGAGGTGGAAACGGCTCCCATTCCTGAAGAAAACCATGTTTGGCTCCAACCGAG
GGTGATGAGACCCACCAAGCCCAAGAAAACCTCTGCGGTCAACTACATGACCCAAGTCGTCAGATGTG
ACACCAAGATGAAGGACAGGTGCAAAGGGTCCACGTGTAACAGGTACCAGTGCCCAGCAGGCTGCCTG
AACCACAAGGCGAAGATCTTTGGAAGTCTGTTCTATGAAAGCTCGTCTAGCATATGCCGCGCCGCCAT
CCACTACGGGATCCTGGATGACAAGGGAGGCCTGGTGGATATCACCAGGAACGGGAAGGTCCCCTTCT
TCGTGAAGTCTGAGAGACACGGCGTGCAGTCCCTCAGCAAATACAAACCTTCCAGCTCATTCATGGTG
TCAAAAGTGAAAGTGCAGGATTTGGACTGCTACACGACCGTTGCTCAGCTGTGCCCGTTTGAAAAGCC
AGCAACTCACTGCCCAAGAATCCATTGTCCGGCACACTGCAAAGACGAACCTTCCTACTGGGCTCCGG
TGTTTGGAACCAACATCTATGCAGATACCTCAAGCATCTGCAAGACAGCCGTGCACGCGGGAGTCATC
AGCAACGAGAGTGGGGGTGACGTGGACGTGATGCCCGTGGATAAAAAGAAGACCTACGTGGGCTCGCT
CAGGAATGGAGTTCAGTCTGAAAGCCTGGGGACTCCTCGGGATGGAAAGGCCTTCCGGATCTTTGCTG
TCAGGCAGTGAATTTCCAGCACCAGGGGAGAAGGGGCGTCTTCAGGAGGGCTTCGGGGTTTTGCTTTT
ATTTTTATTTTGTCATTGCGGGGTATATGGAGAGTCAGGAAACTTCCTTTGACTGATGTTCAGTGTCC
ATCACTTTGTGGCCTGTGGGTGAGGTGACATCTCATCCCCTCACTGAAGCAACAGCATCCCAAGGTGC
TCAGCCGGACTCCCTGGTGCCTGATCCTGCTGGGGCCCGGGGGTCTCCATCTGGACGTCCTCTCTCCT
TTAGAGATCTGAGCTGTCTCTTAAAGGGGACAGTTGCCCAAAATGTTCCTTGCTATGTGTTCTTCTGT
TGGTGGAGGAAGTTGATTTCAACCTCCCTGCCAAAAGAACAAACCATTTGAAGCTCACAATTGTGAAG
CATTCACGGCGTCGGAAGAGGCCTTTTGAGCAAGCGCCAATGAGTTTCAGGAATGAAGTAGAAGGTAG
TTATTTAAAAATAAAAAACACAGTCCGTCCCTACCAATAGAGGAAAATGGTTTTAATGTTTGCTGGTC
AGACAGACAAATGGGCTAGAGTAAGAGGGCTGCGGGTATGAGAGACCCCGGCTCCGCCCTGGCACGTG
TCCTTGCTGGCGGCCCGCCACAGGCCCCCTTCAATGGCCGCATTCAGGATGGCTCTATACACAGCAGT
GCTGGTTTATGTAAAGTTCAGCAGTCACTTCA

| | | | |
|---|---|---|---|
| NOV6h, CG50391-05 | SEQ ID NO: 96 | 501 aa | MW at 56412.8kD |
| Protein Sequence | | | |

MSCVLGGVIPLGLLFLVRGSQGYLLPNVTLLEELLSKYQHNESHSRVRRAIPREDKEEILMLHNKLRG
QVQPQASNMEYMTWDDELEKSAAAWASQCIWEHGPTGLLVSIGQNLGAHWGRYRSPGFHVQSWYDEVK
DYTYPYPSECNPWCPERCSGPMCTHYTQVTQIVWATTNKIGCAVNTCRKMTVWGEVWENAVYFVCNYS
PKRGNWIGEAPYKNGRPCSECPPSYGGSCRNNLCYREETYTPKPETDEMNEVETAPIPEENHVWLQPR
VMRPTKPKKTSAVNYMTQVVRCDTKMKDRCKGSTCNRYQCPAGCLNHKAKIFGSLFYESSSSICRAAI
HYGILDDKGGLVDITRNGKVPFFVKSERHGVQSLSKYKPSSSFMVSKVKVQDLDCYTTVAQLCPFEKP
ATHCPRIHCPAHCKDEPSYWAPVFGTNIYADTSSICKTAVHAGVISNESGGDVDVMPVDKKKTYVGSL
RNGVQSESLGTPRDGKAFRIFAVRQ

| | | |
|---|---|---|
| NOV6i, CG50391-06 | SEQ ID NO: 97 | 1425 bp |
| DNA Sequence | ORF Start: at 1 | ORF Stop: end of sequence |

TACCTCCTGCCCAACGTCACTCTCTTAGAGGAGCTGCTCAGCAAATACCAGCACAACGAGTCTCACTC
CCGGGTCCGCAGAGCCATCCCCAGGGAGGACAAGGAGGAGATCCTCATGCTGCACAACAAGCTTCGGG
GCCAGGTGCAGCCTCAGGCCTCCAACATGGAGTACATGACCTGGGATGACGAACTGGAGAAGTCTGCT
GCAGCGTGGGCCAGTCAGTGCATCTGGGAGCACGGGCCCACCGGTCTGCTGGTGTCCATCGGGCAGAA
CCTGGGCGCTCACTGGGGCAGGTATCGCTCTCCGGGGTTCCATGTGCAGTCCTGGTATGACGAGGTGA
AGGACTACACCTACCCCTACCCGAGCGAGTGCAACCCCTGGTGTCCAGAGAGGTGCTCGGGGCCTATG
TGCACGCACTACACACAGATAGTTTGGGCCACCACCAACAAGATCGGTTGTGCTGTGAACACCTGCCG
GAAGATGACTGTCTGGGGAGAAGTTTGGGAGAACGCGGTCTACTTTGTCTGCAATTATTCTCCAAAGG
GGAACTGGATTGGAGAAGCCCCCTACAAGAATGGCCGGCCCTGCTCTGAGTGCCCACCCAGCTATGGA
GGCAGCTGCAGGAACAACTTGTGTTACCGAGAAGAAACCTACACTCCAAAACCTGAAACGGACGAGAT
GAATGAGGTGGAAACGGCTCCCATTCCTGAAGAAAACCATGTTTGGCTCCAACCGAGGGTGATGAGAC
CCACCAAGCCCAAGAAAACCTCTGCGGTCAACTACATGACCCAAGTCGTCAGATGTGACACCAAGATG
AAGGACAGGTGCAAAGGGTCCACGTGTAACAGGTACCAGTGCCCAGCAGGCTGCCTGAACCACAAGGC
GAAGATCTTTGGAACTCTGTTCTATGAAAGCTCGTCTAGCATATGCCGCGCCGCCATCCACTACGGGA
TCCTGGATGACAAGGGAGGCCTGGTGGATATCACCAGGAACGGGAAGGTCCCCTTCTTCGTGAAGTCT
GAGAGACACGGCGTGCAGTCCCTCAGCAAATACAAACCTTCCAGCTCATTCATGGTGTCAAAAGTGAA
AGTGCAGGATTTGGACTGCTACACGACCGTTGCTCAGCTGTGCCCGTTTGAAAAGCCAGCAACTCACT
GCCCAAGAATCCATTGTCCGGCACACTGCAAAGACGAACCTTCCTACTGGGCTCCGGTGTTTGGAACC
AACATCTATGCAGATACCTCAAGCATCTGCAAGACAGCTGTGCACGCGGGAGTCATCAGCAACGAGAG
TGGGGGTGACGTGGACGTGATGCCCGTGGATAAAAAGAAGACCTACGTGGGCTCGCTCAGGAATGGAG
TTCAGTCTGAAAGCCTGGGGACTCCTCGGGATGGAAAGGCCTTCCGGATCTTTGCTGTCAGGCAG

| | | | |
|---|---|---|---|
| NOV6i, CG50391-06 | SEQ ID NO: 98 | 475 aa | MW at 53743.6kD |
| Protein Sequence | | | |

YLLPNVTLLEELLSKYQHNESHSRVRPAIPREDKEEILMLHNKLRGQVQPQASNMEYMTWDDELEKSA
AAWASQCIWEHGPTGLLVSIGQNLGAHWGRYRSPGFHVQSWYDEVKDYTYPYPSECNPWCPERCSGPM
CTHYTQIVWATTNKIGCAVNTCRKMTVWGEVWENAVYFVCNYSPKGNWIGEAPYKNGRPCSECPPSYG
GSCRNNLCYREETYTPKPETDEMNEVETAPIPEENHVWLQPRVMRPTKPKKTSAVNYMTQVVRCDTKM
KDRCKGSTCNRYQCPAGCLNHKAKIFGTLFYESSSSICRAAIHYGILDDKGGLVDITRNGKVPFFVKS
ERHGVQSLSKYKPSSSFMVSKVKVQDLDCYTTVAQLCPFEKPATHCPRIHCPAHCKDEPSYWAPVFGT
NIYADTSSICKTAVHAGVISNESGGDVDVMPVDKKKTYVGSLRNGVQSESLGTPRDGKAFRIFAVRQ

| | | |
|---|---|---|
| NOV6j, CG50391-07 | SEQ ID NO: 99 | 2272 bp |
| DNA Sequence | ORF Start: ATG at 216 | ORF Stop: TGA at 1707 |

CCCAGGGCGTCTCCGGCTGCTCCCATTGAGCTGTCTGCTCGCTGTGCCCGCTGTGCCTGCTGTGCCCG
CGCTGTCGCCGCTGCTACCGCGTCTGCTGGACGCGGGAGACGCCAGCGAGCTGGTGATTGGAGCCCTG
CGGAGAGCTCAAGCGCCCAGCTCTGCCCNAGGAGCCCAGGCTGCCCCGTGAGTCCCATAGTTGCTGCA
GGAGTGGAGCCATGAGCTGCGTCCTGGGTGGTGTCATCCCCTTGGGGCTGCTGTTCCTGGTCTGCGGA
TCCCAAGGCTACCTCCTGCCCAACGTCACTCTCTTAGAGGAGCTGCTCAGCAAATACCAGCACAACGA
GTCTCACTCCCGGGTCCGCAGAGCCATCCCCAGGGAGGACAAGGAGGAGATCCTCATGCTGCACAACA
AGCTTCGGGGCCAGGTGCAGCCTCAGGCCTCCAACATGGAGTACATGACCTGGGATGACGAACTGGAG
AAGTCTGCTGCAGCGTGGGCCAGTCAGTGCATCTGGGAGCACGGGCCCACCAGTCTGCTGGTGTCCAT
CGGGCAGAACCTGGGCGCTCACTGGGGCAGGTATCGCTCTCCGGGGTTCCATGTGCAGTCCTGGTATG
ACGAGGTGAAGGACTACACCTACCCCTACCCGAGCGAGTGCAACCCCTGGTGTCCAGAGAGGTGCTCG
GGGCCTATGTGCACGCACTACACACAGATAGTTTGGGCCACCACCAACAAGATCGGTTGTGCTGTGAA
CACCTGCCGGAAGATGACTGTCTGGGGAGAAGTTTGGGAGAACGCGGTCTACTTTGTCTGCAATTATT
CTCCAAAGGGGAACTGGATTGGAGAAGCCCCCTACAAGAATGGCCGGCCCTGCTCTGAGTGCCCACCC
AGCTATGGAGGCAGCTGCAGGAACAACTTGTGTTACCGAGAAGAAACCTACACTCCAAAACCTGAAAC
GGACGAGATGAATGAGGTGGAAACGGCTCCCATTCCTGAAGAAAACCATGTTTGGCTCCAACCGAGGG
TGATGAGACCCACCAAGCCCAAGAAAACCTCTGCGGTCAACTACATGACCCAAGTCGTCAGATGTGAC
ACCAAGATGAAGGACAGGTGCAAAGGGTCCACGTGTAACAGGTACCAGTGCCCAGCAGGCTGCCTGAA
CCACAAGGCGAAGATCTTTGGAAGTCTGTTCTATGAAAGCTCGTCTAGCATATGCCGCGCCGCCATCC
ACTACGGGATCCTGGATGACAAGGGAGGCCTGGTGGATATCACCAGGAACGGGAAGGTCCCCTTCTTC
GTGAAGTCTGAGAGACACGGCGTGCAGTCCCTCAGCAAATACAAACCTTCCAGCTCATTCATGGTGTC
AAAAGTGAAAGTGCAGGATTTGGACTGCTACACGACCGTTGCTCAGCTGTGCCCGTTTGAAAAGCCAG
CAACTCACTGCCCAAGAATCCATTGTCCGGCACACTGCAAAGACGAACCTTCCTACTGGGCTCCGGTG
TTTGGAACCAACATCTATGCAGATACCTCAAGCATCTGCAAGACAGCTGTGCACGCGGGAGTCATCAG
CAACGAGAGTGGGGGTGACGTGGACGTGATGCCCGTGGATAAAAAGAAGACCTACGTGGGCTCGCTCA
GGAATGGAGTTCAGTCTGAAAGCCTGGGGACTCCTCGGGATGGAAAGGCCTTCCGGATCTTTGCTGTC
AGGCAGTGAATTTCCAGCACCAGGGGAGAAGGGGCGTCTTCAGGAGGGCTTCGGGGTTTTGCTTTTAT
TTTTATTTTGTCATTGCGGGGTATATGGAGAGTCAGGAAACTTCCTTTGACTGATGTTCAGTGTCCAT
CACTTTGTGGCCTGTGGGTGAGGTGACATCTCATCCCCTCACTGAAGCAACAGCATCCCAAGGTGCTC
AGCCGGACTCCCTGGTGCCTGATCCTGCTGGGGCCTGGGGGTCTCCATCTGGACGTCCTCTCTCCTTT
AGAGATCTGAGCTGTCTCTTAAAGGGGACAGTTGCCCAAAATGTTCCTTGCTATGTGTTCTTCTGTTG
GTGGAGGAAGTTGATTTCAACCCCCCTGCCAAAAGAACAAACCATTTGAAGCTCACAATTGTGAAGCA
TTCACGGCGTCGGAAGAGGCCTTTTGAGCAAGCGCCAATGAGTTTCAGGAATGAAGTAGAAGGTAGTT
ATTTAAAAATAAAAAACACAGTCCGTCCCTACCAATAGAGGAAAATGGTTTTAATGTTTGCTGGTCAG
ACAGACAAATGGGCTAGAGTAAGAAGGC

| | | | |
|---|---|---|---|
| NOV6j, CG50391-07 | SEQ ID NO: 100 | 497 aa | MW at 55905.2kD |
| Protein Sequence | | | |

MSCVLGGVIPLGLLFLVCGSQGYLLPNVTLLEELLSKYQHNESHSRVRRAIPREDKEEILMLHNKLRG
QVQPQASNMEYMTWDDELEKSAAAWASQCIWEHGPTSLLVSIGQNLGAHWGRYRSPGFHVQSWYDEVK
DYTYPYPSECNPWCPERCSGPMCTHYTQIVWATTNKIGCAVNTCRKMTVWGEVWENAVYFVCNYSPKG
NWIGEAPYKNGRPCSECPPSYGGSCRNNLCYREETYTPKPETDEMNEVETAPIPEENHVWLQPRVMRP
TKPKKTSAVNYMTQVVRCDTKMKDRCKGSTCNRYQCPAGCLNHKAKIFGSLFYESSSSICRAAIHYGI
LDDKGGLVDITRNGKVPFFVKSERHGVQSLSKYKPSSSFMVSKVKVQDLDCYTTVAQLCPFEKPATHC
PRIHCPAHCKDEPSYWAPVFGTNIYADTSSICKTAVHAGVISNESGGDVDVMPVDKKKTYVGSLRNGV
QSESLGTPRDGKAFRIFAVRQ

| | | |
|---|---|---|
| NOV6k, CG50391-11 | SEQ ID NO: 101 | 1612 bp |
| DNA Sequence | ORF Start: ATG at 31 | ORF Stop: TGA at 1522 |

GAGTCCCATAGTTGCTGCAGGAGTGGAGCCATGAGCTGCGTCCTGGGTGGTGTCATCCCCTTGGGGCT
GCTGTTCCTGGTCTGCGGATCCCAAGGCTACCTCCTGCCCAACGTCACTCTCTTAGAGGAGCTGCTCA
GCAAATACCAGCACAACGAGTCTCACTCCCGGGTCCGCAGAGCCATCCCCAGGGAGGACAAGGAGGAG
ATCCTCATGCTGCACAACAAGCTTCGGGGCCAGGTGCAGCCTCAGGCCTCCAACATGGAGTACATGAC
CTGGGATGACGAACTGGAGAAGTCTGCTGCAGCGTGGGCCAGTCAGTGCATCTGGGAGCACGGGCCCA
CCAGTCTGCTGGTGTCCATCGGGCAGAACCTGGGCGCTCACTGGGGCAGGTATCGCTCTCCGGGGTTC
CATGTGCAGTCCTGGTATGACGAGGTGAAGGACTACACCTACCCCTACCCGAGCGAGTGCAACCCCTG
GTGTCCAGAGAGGTGCTCGGGGCCCATGTGCACGCACTACACACAGATAGTTTGGGCCACCACCAACA
AGATCGGTTGTGCTGTGAACACCTGCCGGAAGATGACTGTCTGGGGAGAAGTTTGGGAGAACGCGGTC
TACTTTGTCTGCAATTATTCTCCAAAGGGGAACTGGATTGGAGAAGCCCCCTACAAGAATGGCCGGCC
CTGCTCTGAGTGCCCACCCAGCTATGGAGGCAGCTGCAGGAACAACTTGTGTTACCGAGAAGAAACCT
ACACTCCAAAACCTGAAACGGACGAGATGAATGAGGTGGGAACGGCTCCCATTCCTGAAGAAAACCAT
GTTTGGCTCCAACCGAGGGTGATGAGACCCACCAAGCCCAAGAAAACCTCTGCGGTCAACTACATGAC
CCAAGTCGTCAGATGTGACACCAAGATGAAGGACAGGTGCAAAGGGTCCACGTGTAACAGGTACCAGT
GCCCAGCAGGCTGCCTGAACCACAAGGCGAAGATCTTTGGAACTCTGTTCTATGAAAGCTCGTCTAGC
ATATGCCGCGCCGCCATCCACTACGGGATCCTGGATGACAAGGGAGGCCTGGTGGATATCACCAGGAA
CGGGAAGGTCCCCTTCTTCGTGAAGTCTGAGAGACACGGCGTGCAGTCCCTCAGCAAATACAAACCTT
CCAGCTCATTCATGGTGTCAAAAGTGAAAGTGCAGGATTTGGACTGCTACACGACCGTTGCTCAGCTG
TGCCCGTTTGAAAAGCCAGCAACTCACTGCCCAAGAATCCATTGTCCGGCACACTGCAAAGACGAACC
TTCCTACTGGGCTCCGGTGTTTGGAACCAACATCTATGCAGATACCTCAAGCATCTGCAAGACAGCCG
TGCACGCGGGAGTCATCAGCAACGAGAGTGGGGGTGACGTGGACGTGATGCCCGTGGATAAAAAGAAG
ACCTACGTGGGCTCGCTCAGGAATGGAGTTCAGTCTGAAAGCCTGGGGACTCCTCGGGATGGAAAGGC
CTTCCGGATCTTTGCTGTCAGGCAGTGAATTTCCAGCACCAGGGGAGAAGGGGCGTCTTCAGGAGGGC
TTCGGGGTTTTGCTTTTATTTTTATTTTGTCATTGCGGGGTATAGGAG

| | | | |
|---|---|---|---|
| NOV6k, CG50391-11 | SEQ ID NO: 102 | 497 aa | MW at 55847.2kD |
| Protein Sequence | | | |

MSCVLGGVIPLGLLFLVCGSQGYLLPNVTLLEELLSKYQHNESHSRVRRAIPREDKEEILMLHNKLRG
QVQPQASNMEYMTWDDELEKSAAAWASQCIWEHGPTSLLVSIGQNLGAHWGRYRSPGFHVQSWYDEVK
DYTYPYPSECNPWCPERCSGPMCTHYTQIVWATTNKIGCAVNTCRKMTVWGEVWENAVYFVCNYSPKG
NWIGEAPYKNGRPCSECPPSYGGSCRNNLCYREETYTPKPETDEMNEVGTAPIPEENHVWLQPRVMRP
TKPKKTSAVNYMTQVVRCDTKMKDRCKGSTCNRYQCPAGCLNHKAKIFGTLFYESSSSICRAATHYGI
LDDKGGLVDITRNGKVPFFVKSERHGVQSLSKYKPSSSFMVSKVKVQDLDCYTTVAQLCPFEKPATHC
PRIHCPAHCKDEPSYWAPVFGTNIYADTSSICKTAVHAGVISNESGGDVDVMPVDKKKTYVGSLRNGV
QSESLGTPRDGKAFRIFAVRQ

| | | |
|---|---|---|
| NOV6l, SNP13376336 of | SEQ ID NO: 103 | 2268 bp |
| CG50391-08, DNA Sequence | ORF Start: ATG at 206 | ORF Stop: TGA at 1565 |
| | SNP Pos: 393 | SNP Change: A to G |

CTCTGACTGCTCCTATTGAGCTGTCTGCTCGCTGTGCCCGCTGTGCCTGCTGTGCCCGCGCTGTCGCC
GCTGCTACCGCGTCTACTGGACGCGGGAGACGCCAGCGAGCTGGTGATTGGAGCCCTGCGGAGAGCTC
AAGCGCCCAGCTCTGCCCGAGGAGCCCAGGCTGCCCCGTGAGTCCCATAGTTGCTGCAGGAGTGGAGC
CATGAGCTGCGTCCTGGGTGGTGTCATCCCCTTGGGGCTGCTGTTCCTGGTCTGCGGATCCCAAGGCT
ACCTCCTGCCCAACGTCACTCTCTTAGAGGAGCTGCTCAGCAAATACCAGCACAACGAGTCTCACTCC
CGGGTCCGCAGAGCCATCCCCAGGGAGGACAAGGAGGAGATCCTCATGCTGCGCAACAAGCTTCGGGG
CCAGGTGCAGCCTCAGGCCTCCAACATGGAGTACATGACCTGGGATGACGAACTGGAGAAGTCTGCTG
CAGCGTGGGCCAGTCAGTGCATCTGGGAGCACGGGCCCACCAGTCTGCTGGTGTCCATCGGGCAGAAC
CTGGGCGCTCACTGGGGCAGAATAGTTTGGGTCACCACCAACAAGATCGGTTGTGCTGTGAACACCTG
CCGGAAGATGACTGTCTGGGGAGAAGTTTGGGAGAACGCGGTCTACTTTGTCTGCAATTATTCTCCAA
AGGGGAACTGGATTGGAGAAGCCCCCTACAAGAATGGCCGGCCCTGCTCTGAGTGCCCACCCAGCTAT
GGAGGCAGCTGCAGGAACAACTTGTGTTACCGAGAAGAAACCTACACTCCAAAACCTGAAACGGACGA
GATGAATGAGGTGGAAACGGCTCCCATTCCTGAAGAAAACCATGTTTGGCTCCAACCGAGGGTGATGA
GACCCACCAAGCCCAAGAAAACCTCTGCGGTCAACTACATGACCCAAGTCGTCAGATGTGACACCAAG
ATGAAGGACAGGTGCAAAGGGTCCACGTGTAACAGGTACCAGTGCCCAGCAGGCTGCCTGAACCACAA
GGCGAAGATCTTTGGAAGTCTGTTCTATGAAAGCTCGTCTAGCATATGCCGCGCCGCCATCCACTACG
GGATCCTGGATGACAAGGGAGGCCTGGTGGATATCACCAGGAACGGGAAGGTCCCCTTCTTCGTGAAG
TCTGAGAGACACGGCGTGCAGTCCCTCAGCAAATACAAACCTTCCAGCTCATTCATGGTGTCAAAAGT
GAAAGTGCAGGATTTGGACTGCTACACGACCGTTGCTCAGCTGTGCCCGTTTGAAAAGCCAGCAACTC
ACTGCCCAAGAATCCATTGTCCGGCACACTGCAAAGACGAACCTTCCTACTGGGCTCCGGTGTTTGGA
ACCAACATCTATGCAGATACCTCAAGCATCTGCAAGACAGCCGTGCACGCGGGAGTCATCAGCAACGA
GAGTGGGGGTGACGTGGACGTGATGCCCGTGGATAAAAAGAAGACCTACGTGGGCTCGCTCAGGAATG
GAGTTCAGTCTGAAAGCCTGGGGACTCCTCGGGATGGAAAGGCCTTCCGGATCTTTGCTGTCAGGCAG
TGAATTTCCAGCACCAGGGGAGAAGGGGCGTCTTCAGGAGGGCTTCGGGGTTTTGCTTTTATTTTTAT
TTTGTCATTGCGGGGTATATGGAGAGTCAGGAAACTTCCTTTGACTGATGTTCAGTGTCCATCACTTT
GTGGCCTGTGGGTGAGGTGACATCTCATCCCCTCACTGAAGCAACAGCATCCCAAGGTGCTCAGCCGG
ACTCCCTGGTGCCTGATCCTGCTGGGGCCCGGGGGTCTCCATCTGGACGTCCTCTCTCCTTTAGAGAT
CTGAGCTGTCTCTTAAAGGGGACAGTTGCCCAAAATGTTCCTTGCTATGTGTTCTTCTGTTGGTGGAC
GAAGTTGATTTCAACCTCCCTGCCAAAAGAACAAACCATTTGAAGCTCACAATTGTGAAGCATTCACG
GCGTCGGAAGAGGCCTTTTGAGCAAGCGCCAATGAGTTTCAGGAATGAAGTAGAAGGTAGTTATTTAA
AAATAAAAAACACAGTCCGTCCCTACCAATAGAGGAAAATGGTTTTAATGTTTGCTGGTCAGACAGAC
AAATGGGCTAGAGTAAGAGGGCTGCGGGTATGAGAGACCCCGGCTCCGCCCTGGCACGTGTCCTTGCT
GGCGGCCCGCCACAGGCCCCCTTCAATGGCCGCATTCAGGATGGCTCTATACACAGCAGTGCTGGTTT
ATGTAAAGTTCAGCAGTCACTTCA

| | | | |
|---|---|---|---|
| NOV6l, SNP13376336 of | SEQ ID NO: 104 | 453 aa | MW at 50660.5kD |
| CG50391-08, Protein | SNP Pos: 63 | | SNP Change: His to Arg |

MSCVLGGVIPLGLLFLVCGSQGYLLPNVTLLEELLSKYQHNESHSRVRRAIPREDKEEILMLRNKLRG
QVQPQASNMEYMTWDDELEKSAAAWASQCIWEHGPTSLLVSIGQNLGAHWGRIVWVTTNKIGCAVNTC
RKMTVWGEVWENAVYFVCNYSPKGNWIGEAPYKNGRPCSECPPSYGGSCRNNLCYREETYTPKPETDE
MNEVETAPIPEENHVWLQPRVMRPTKPKKTSAVNYMTQVVRCDTKMKDRCKGSTCNRYQCPAGCLNHK
AKIFGSLFYESSSSICRAAIHYGILDDKGGLVDITRNGKVPFFVKSERHGVQSLSKYKPSSSFMVSKV
KVQDLDCYTTVAQLCPFEKPATHCPRIHCPAHCKDEPSYWAPVFGTNIYADTSSICKTAVHAGVISNE
SGGDVDVMPVDKKKTYVGSLRNGVQSESLGTPRDGKAFRIFAVRQ

| | | |
|---|---|---|
| NOV6m, SNP13376335 of | SEQ ID NO: 105 | 2268 bp |
| CG50391-08, DNA Sequence | ORF Start: ATG at 206 | ORF Stop: TGA at 1565 |
| | SNP Pos: 477 | SNP Change: C to T |

CTCTGACTGCTCCTATTGAGCTGTCTGCTCGCTGTGCCCGCTGTGCCTGCTGTGCCCGCGCTGTCGCC
GCTGCTACCGCGTCTACTGGACGCGGGAGACGCCAGCGAGCTGGTGATTGGAGCCCTGCGGAGAGCTC
AAGCGCCCAGCTCTGCCCGAGGAGCCCAGGCTGCCCCGTGAGTCCCATAGTTGCTGCAGGAGTGGAGC
CATGAGCTGCGTCCTGGGTGGTGTCATCCCCTTGGGGCTGCTGTTCCTGGTCTGCGGATCCCAAGGCT
ACCTCCTGCCCAACGTCACTCTCTTAGAGGAGCTGCTCAGCAAATACCAGCACAACGAGTCTCACTCC
CGGGTCCGCAGAGCCATCCCCAGGGAGGACAAGGAGGAGATCCTCATGCTGCACAACAAGCTTCGGGG
CCAGGTGCAGCCTCAGGCCTCCAACATGGAGTACATGACCTGGGATGACGAACTGGAGAAGTCTGCTG
TAGCGTGGGCCAGTCAGTGCATCTGGGAGCACGGGCCCACCAGTCTGCTGGTGTCCATCGGGCAGAAC
CTGGGCGCTCACTGGGGCAGAATAGTTTGGGTCACCACCAACAAGATCGGTTGTGCTGTGAACACCTG
CCGGAAGATGACTGTCTGGGGAGAAGTTTGGGAGAACGCGGTCTACTTTGTCTGCAATTATTCTCCAA
AGGGGAACTGGATTGGAGAAGCCCCCTACAAGAATGGCCGGCCCTGCTCTGAGTGCCCACCCAGCTAT
GGAGGCAGCTGCAGGAACAACTTGTGTTACCGAGAAGAAACCTACACTCCAAAACCTGAAACGGACGA
GATGAATGAGGTGGAAACGGCTCCCATTCCTGAAGAAAACCATGTTTGGCTCCAACCGAGGGTGATGA
GACCCACCAAGCCCAAGAAAACCTCTGCGGTCAACTACATGACCCAAGTCGTCAGATGTGACACCAAG
ATGAAGGACAGGTGCAAAGGGTCCACGTGTAACAGGTACCAGTGCCCAGCAGGCTGCCTGAACCACAA
GGCGAAGATCTTTGGAAGTCTGTTCTATGAAAGCTCGTCTAGCATATGCCGCGCCGCCATCCACTACG
GGATCCTGGATGACAAGGGAGGCCTGGTGGATATCACCAGGAACGGGAAGGTCCCCTTCTTCGTGAAG
TCTGAGAGACACGGCGTGCAGTCCCTCAGCAAATACAAACCTTCCAGCTCATTCATGGTGTCAAAAGT
GAAAGTGCAGGATTTGGACTGCTACACGACCGTTGCTCAGCTGTGCCCGTTTGAAAAGCCAGCAACTC
ACTGCCCAAGAATCCATTGTCCGGCACACTGCAAAGACGAACCTTCCTACTGGGCTCCGGTGTTTGGA
ACCAACATCTATGCAGATACCTCAAGCATCTGCAAGACAGCCGTGCACGCGGGAGTCATCAGCAACGA
GAGTGGGGGTGACGTGGACGTGATGCCCGTGGATAAAAAGAAGACCTACGTGGGCTCGCTCAGGAATG
GAGTTCAGTCTGAAAGCCTGGGGACTCCTCGGGATGGAAAGGCCTTCCGGATCTTTGCTGTCAGGCAG
TGAATTTCCAGCACCAGGGGAGAAGGGGCGTCTTCAGGAGGGCTTCGGGGTTTTGCTTTTATTTTTAT
TTTGTCATTGCGGGGTATATGGAGAGTCAGGAAACTTCCTTTGACTGATGTTCAGTGTCCATCACTTT
GTGGCCTGTGGGTGAGGTGACATCTCATCCCCTCACTGAAGCAACAGCATCCCAAGGTGCTCAGCCGG
ACTCCCTGGTGCCTGATCCTGCTGGGGCCCGGGGGTCTCCATCTGGACGTCCTCTCTCCTTTAGAGAT
CTGAGCTGTCTCTTAAAGGGGACAGTTGCCCAAAATGTTCCTTGCTATGTGTTCTTCTGTTGGTGGAG
GAAGTTGATTTCAACCTCCCTGCCAAAAGAACAAACCATTTGAAGCTCACAATTGTGAAGCATTCACG
GCGTCGGAAGAGGCCTTTTGAGCAAGCGCCAATGAGTTTCAGGAATGAAGTAGAAGGTAGTTATTTAA
AAATAAAAAACACAGTCCGTCCCTACCAATAGAGGAAAATGGTTTTAATGTTTGCTGGTCAGACAGAC
AAATGGGCTAGAGTAAGAGGGCTGCGGGTATGAGAGACCCCGGCTCCGCCCTGGCACGTGTCCTTGCT
GGCGGCCCGCCACAGGCCCCCTTCAATGGCCGCATTCAGGATGGCTCTATACACAGCAGTGCTGGTTT
ATGTAAAGTTCAGCAGTCACTTCA

| | | | |
|---|---|---|---|
| NOV6m, SNP13376335 of | SEQ ID NO: 106 | 453 aa | MW at 50669.5kD |
| CG50391-08, Protein | SNP Pos: 91 | | SNP Change: Ala to Val |
| Sequence | | | |

MSCVLGGVIPLGLLFLVCGSQGYLLPNVTLLEELLSKYQHNESHSRVRRAIPREDKEEILMLHNKLRG
QVQPQASNMEYMTWDDELEKSAVAWASQCIWEHGPTSLLVSIGQNLGAHWGRIVWVTTNKIGCAVNTC
RKMTVWGEVWENAVYFVCNYSPKGNWIGEAPYKNGRPCSECPPSYGGSCRNNLCYREETYTPKPETDE
MNEVETAPIPEENHVWLQPRVMRPTKPKKTSAVNYMTQVVRCDTKMKDRCKGSTCNRYQCPAGCLNHK
AKIFGSLFYESSSSICRAAIHYGILDDKGGLVDITPNGKVPFFVKSERHGVQSLSKYKPSSSFMVSKV
KVQDLDCYTTVAQLCPFEKPATHCPRIHCPAHCKDEPSYWAPVFGTNIYADTSSICKTAVHAGVISNE
SGGDVDVMPVDKKKTYVGSLRNGVQSESLGTPRDGKAFRIFAVRQ

| | | |
|---|---|---|
| NOV6n, SNP13376334 of | SEQ ID NO: 107 | 2268 bp |
| CG50391-08, DNA Sequence | ORF Start: ATG at 206 | ORF Stop: TGA at 1565 |
| | SNP Pos: 798 | SNP Change: C to T |

CTCTGACTGCTCCTATTGAGCTGTCTGCTCGCTGTGCCCGCTGTGCCTGCTGTGCCCGCGCTGTCGCC
GCTGCTACCGCGTCTACTGGACGCGGGAGACGCCAGCGAGCTGGTGATTGGAGCCCTGCGGAGAGCTC
AAGCGCCCAGCTCTGCCCGAGGAGCCCAGGCTGCCCCGTGAGTCCCATAGTTGCTGCAGGAGTGGAGC
CATGAGCTGCGTCCTGGGTGGTGTCATCCCCTTGGGGCTGCTGTTCCTGGTCTGCGGATCCCAAGGCT
ACCTCCTGCCCAACGTCACTCTCTTAGAGGAGCTGCTCAGCAAATACCAGCACAACGAGTCTCACTCC
CGGGTCCGCAGAGCCATCCCCAGGGAGGACAAGGAGGAGATCCTCATGCTGCACAACAAGCTTCGGGG
CCAGGTGCAGCCTCAGGCCTCCAACATGGAGTACATGACCTGGGATGACGAACTGGAGAAGTCTGCTG
CAGCGTGGGCCAGTCAGTGCATCTGGGAGCACGGGCCCACCAGTCTGCTGGTGTCCATCGGGCAGAAC
CTGGGCGCTCACTGGGGCAGAATAGTTTGGGTCACCACCAACAAGATCGGTTGTGCTGTGAACACCTG
CCGGAAGATGACTGTCTGGGGAGAAGTTTGGGAGAACGCGGTCTACTTTGTCTGCAATTATTCTCCAA
AGGGGAACTGGATTGGAGAAGCCCCCTACAAGAATGGCCGGCCCTGCTCTGAGTGCCCACCCAGCTAT
GGAGGCAGCTGCAGGAACAACTTGTGTTACCGAGAAGAAACCTACACTCTAAAACCTGAAACGGACGA
GATGAATGAGGTGGAAACGGCTCCCATTCCTGAAGAAAACCATGTTTGGCTCCAACCGAGGGTGATGA
GACCCACCAAGCCCAAGAAAACCTCTGCGGTCAACTACATGACCCAAGTCGTCAGATGTGACACCAAG
ATGAAGGACAGGTGCAAAGGGTCCACGTGTAACAGGTACCAGTGCCCAGCAGGCTGCCTGAACCACAA
GGCGAAGATCTTTGGAAGTCTGTTCTATGAAAGCTCGTCTAGCATATGCCGCGCCGCCATCCACTACG
GGATCCTGGATGACAAGGGAGGCCTGGTGGATATCACCAGGAACGGGAAGGTCCCCTTCTTCGTGAAG
TCTGAGAGACACGGCGTGCAGTCCCTCAGCAAATACAAACCTTCCAGCTCATTCATGGTGTCAAAAGT
GAAAGTGCAGGATTTGGACTGCTACACGACCGTTGCTCAGCTGTGCCCGTTTGAAAAGCCAGCAACTC
ACTGCCCAAGAATCCATTGTCCGGCACACTGCAAAGACGAACCTTCCTACTGGGCTCCGGTGTTTGGA
ACCAACATCTATGCAGATACCTCAAGCATCTGCAAGACAGCCGTGCACGCGGGAGTCATCAGCAACGA
GAGTGGGGGTGACGTGGACGTGATGCCCGTGGATAAAAAGAAGACCTACGTGGGCTCGCTCAGGAATG
GAGTTCAGTCTGAAAGCCTGGGGACTCCTCGGGATGGAAAGGCCTTCCGGATCTTTGCTGTCAGGCAG
TGAATTTCCAGCACCAGGGGAGAAGGGGCGTCTTCAGGAGGGCTTCGGGGTTTTGCTTTTATTTTTAT
TTTGTCATTGCGGGGTATATGGAGAGTCAGGAAACTTCCTTTGACTGATGTTCAGTGTCCATCACTTT
GTGGCCTGTGGGTGAGGTGACATCTCATCCCCTCACTGAAGCAACAGCATCCCAAGGTGCTCAGCCGG
ACTCCCTGGTGCCTGATCCTGCTGGGGCCCGGGGGTCTCCATCTGGACGTCCTCTCTCCTTTAGAGAT
CTGAGCTGTCTCTTAAAGGGGACAGTTGCCCAAAATGTTCCTTGCTATGTGTTCTTCTGTTGGTGGAG
GAAGTTGATTTCAACCTCCCTGCCAAAAGAACAAACCATTTGAAGCTCACAATTGTGAAGCATTCACG
GCGTCGGAAGAGGCCTTTTGAGCAAGCGCCAATGAGTTTCAGGAATGAAGTAGAAGGTAGTTATTTAA
AAATAAAAAACACAGTCCGTCCCTACCAATAGAGGAAAATGGTTTTAATGTTTGCTGGTCAGACAGAC
AAATGGGCTAGAGTAAGAGGGCTGCGGGTATGAGAGACCCCGGCTCCGCCCTGGCACGTGTCCTTGCT
GGCGGCCCGCCACAGGCCCCCTTCAATGGCCGCATTCAGGATGGCTCTATACACAGCAGTGCTGGTTT
ATGTAAAGTTCAGCAGTCACTTCA

| | | | |
|---|---|---|---|
| NOV6n, SNP13376334 of | SEQ ID NO: 108 | 453 aa | MW at 50657.5kD |
| CG50391-08, Protein | SNP Pos: 198 | SNP Change: Pro to Leu | |
| Sequence | | | |

MSCVLGGVIPLGLLFLVCGSQGYLLPNVTLLEELLSKYQHNESHSRVRRAIPREDKEEILMLHNKLRG
QVQPQASNMEYMTWDDELEKSAAAWASQCIWEHGPTSLLVSIGQNLGAHWGRIVWVTTNKIGCAVNTC
RKMTVWGEVWENAVYFVCNYSPKGNWIGEAPYKNGRPCSECPPSYGGSCRNNLCYREETYTLKPETDE
MNEVETAPIPEENHVWLQPRVMRPTKPKKTSAVNYMTQVVRCDTKMKDRCKGSTCNRYQCPAGCLNHK
AKIFGSLFYESSSSICRAAIHYGILDDKGGLVDITRNGKVPFFVKSERHGVQSLSKYKPSSSFMVSKV
KVQDLDCYTTVAQLCPFEKPATHCPRIHCPAHCKDEPSYWAPVFGTNIYADTSSICKTAVHAGVISNE
SGGDVDVMPVDKKKTYVGSLRNGVQSESLGTPRDGKAFRIFAVRQ

| | | |
|---|---|---|
| NOV6o, SNP13376333 of | SEQ ID NO: 109 | 2268 bp |
| CG50391-08, DNA Sequence | ORF Start: ATG at 206 | ORF Stop: TGA at 1565 |
| | SNP Pos: 816 | SNP Change: A to G |

CTCTGACTGCTCCTATTGAGCTGTCTGCTCGCTGTGCCCGCTGTGCCTGCTGTGCCCGCGCTGTCGCC
GCTGCTACCGCGTCTACTGGACGCGGGAGACGCCAGCGAGCTGGTGATTGGAGCCCTGCGGAGAGCTC
AAGCGCCCAGCTCTGCCCGAGGAGCCCAGGCTGCCCCGTGAGTCCCATAGTTGCTGCAGGAGTGGAGC
CATGAGCTGCGTCCTGGGTGGTGTCATCCCCTTGGGGCTGCTGTTCCTGGTCTGCGGATCCCAAGGCT
ACCTCCTGCCCAACGTCACTCTCTTAGAGGAGCTGCTCAGCAAATACCAGCACAACGAGTCTCACTCC
CGGGTCCGCAGAGCCATCCCCAGGGAGGACAAGGAGGAGATCCTCATGCTGCACAACAAGCTTCGGGG
CCAGGTGCAGCCTCAGGCCTCCAACATGGAGTACATGACCTGGGATGACGAACTGGAGAAGTCTGCTG
CAGCGTGGGCCAGTCAGTGCATCTGGGAGCACGGGCCCACCAGTCTGCTGGTGTCCATCGGGCAGAAC
CTGGGCGCTCACTGGGGCAGAATAGTTTGGGTCACCACCAACAAGATCGGTTGTGCTGTGAACACCTG
CCGGAAGATGACTGTCTGGGGAGAAGTTTGGGAGAACGCGGTCTACTTTGTCTGCAATTATTCTCCAA
AGGGGAACTGGATTGGAGAAGCCCCCTACAAGAATGGCCGGCCCTGCTCTGAGTGCCCACCCAGCTAT
GGAGGCAGCTGCAGGAACAACTTGTGTTACCGAGAAGAAACCTACACTCCAAAACCTGAAACGGACGG
GATGAATGAGGTGGAAACGGCTCCCATTCCTGAAGAAAACCATGTTTGGCTCCAACCGAGGGTGATGA
GACCCACCAAGCCCAAGAAAACCTCTGCGGTCAACTACATGACCCAAGTCGTCAGATGTGACACCAAG
ATGAAGGACAGGTGCAAAGGGTCCACGTGTAACAGGTACCAGTGCCCAGCAGGCTGCCTGAACCACAA
GGCGAAGATCTTTGGAAGTCTGTTCTATGAAAGCTCGTCTAGCATATGCCGCGCCGCCATCCACTACG
GGATCCTGGATGACAAGGGAGGCCTGGTGGATATCACCAGGAACGGGAAGGTCCCCTTCTTCGTGAAG
TCTGAGAGACACGGCGTGCAGTCCCTCAGCA~ATACAAACCTTCCAGCTCATTCATGGTGTCAAAAGT
GAAAGTGCAGGATTTGGACTGCTACACGACCGTTGCTCAGCTGTGCCCGTTTGAAAAGCCAGCAACTC
ACTGCCCAAGAATCCATTGTCCGGCACACTGCAAAGACGAACCTTCCTACTGGGCTCCGGTGTTTGGA
ACCAACATCTATGCAGATACCTCAAGCATCTGCAAGACAGCCGTGCACGCGGGAGTCATCAGCAACGA
GAGTGGGGGTGACGTGGACGTGATGCCCGTGGATAAAAAGAAGACCTACGTGGGCTCGCTCAGGAATG
GAGTTCAGTCTGAAAGCCTGGGGACTCCTCGGGATGGAAAGGCCTTCCGGATCTTTGCTGTCAGGCAG
TGAATTTCCAGCACCAGGGGAGAAGGGGCGTCTTCAGGAGGGCTTCGGGGTTTTGCTTTTATTTTTAT
TTTGTCATTGCGGGGTATATGGAGAGTCAGGAAACTTCCTTTGACTGATGTTCAGTGTCCATCACTTT
GTGGCCTGTGGGTGAGGTGACATCTCATCCCCTCACTGAAGCAACAGCATCCCAAGGTGCTCAGCCGG
ACTCCCTGGTGCCTGATCCTGCTGGGGCCCGGGGGTCTCCATCTGGACGTCCTCTCTCCTTTAGAGAT
CTGAGCTGTCTCTTAAAGGGGACAGTTGCCCAAAATGTTCCTTGCTATGTGTTCTTCTGTTGGTGGAG
GAAGTTGATTTCAACCTCCCTGCCAAAAGAACAAACCATTTGAAGCTCACAATTGTGAAGCATTCACG
GCGTCGGAAGAGGCCTTTTGAGCAAGCGCCAATGAGTTTCAGGAATGAAGTAGAAGGTAGTTATTTAA
AAATAAAAAACACAGTCCGTCCCTACCAATAGAGGAAAATGGTTTTAATGTTTGCTGGTCAGACAGAC
AAATGGGCTAGAGTAAGAGGGCTGCGGGTATGAGAGACCCCGGCTCCGCCCTGGCACGTGTCCTTGCT
GGCGGCCCGCCACAGGCCCCCTTCAATGGCCGCATTCAGGATGGCTCTATACACAGCAGTGCTGGTTT
ATGTAAAGTTCAGCAGTCACTTCA

| | | | |
|---|---|---|---|
| NOV6o, SNP13376333 of | SEQ ID NO: 110 | 453 aa | MW at 50569.4kD |
| CG50391-08, Protein | SNP Pos: 204 | | SNP Change: Glu to Gly |
| Sequence | | | |

MSCVLGGVIPLGLLFLVCGSQGYLLPNVTLLEELLSKYQHNESHSRVRPAIPREDKEEILMLHNKLRG
QVQPQASNNEYMTWDDELEKSAAAWASQCIWEHGPTSLLVSIGQNLGAHWGRIVWVTTNKIGCAVNTC
RKMTVWGEVWENAVYFVCNYSPKGNWIGEAPYKNGRPCSECPPSYGGSCRNNLCYREETYTPKPETDG
MNEVETAPIPEENHVWLQPRVMRPTKPKKTSAVNYMTQVVRCDTKMKDRCKGSTCNRYQCPAGCLNHK
AKIFGSLFYESSSSICRAAIHYGILDDKGGLVDITRNGKVPFFVKSERHGVQSLSKYKPSSSFMVSKV
KVQDLDCYTTVAQLCPFEKPATHCPRIHCPAHCKDEPSYWAPVFGTNIYADTSSICKTAVHAGVISNE
SGGDVDVMPVDKKKTYVGSLRNGVQSESLGTPRDGKAFRIFAVRQ

| | | |
|---|---|---|
| NOV6p, SNP13382488 of | SEQ ID NO: 111 | 2268 bp |
| CG50391-08, DNA Sequence | ORF Start: ATG at 206 | ORF Stop: TGA at 1565 |
| | SNP Pos: 1402 | SNP Change: C to T |

CTCTGACTGCTCCTATTGAGCTGTCTGCTCGCTGTGCCCGCTGTGCCTGCTGTGCCCGCGCTGTCGCC
GCTGCTACCGCGTCTACTGGACGCGGGAGACGCCAGCGAGCTGGTGATTGGAGCCCTGCGGAGAGCTC
AAGCGCCCAGCTCTGCCCGAGGAGCCCAGGCTGCCCCGTGAGTCCCATAGTTGCTGCAGGAGTGGAGC
CATGAGCTGCGTCCTGGGTGGTGTCATCCCCTTGGGGCTGCTGTTCCTGGTCTGCGGATCCCAAGGCT
ACCTCCTGCCCAACGTCACTCTCTTAGAGGAGCTGCTCAGCAAATACCAGCACAACGAGTCTCACTCC
CGGGTCCGCAGAGCCATCCCCAGGGAGGACAAGGAGGAGATCCTCATGCTGCACAACAAGCTTCGGGG
CCAGGTGCAGCCTCAGGCCTCCAACATGGAGTACATGACCTGGGATGACGAACTGGAGAAGTCTGCTG
CAGCGTGGGCCAGTCAGTGCATCTGGGAGCACGGGCCCACCAGTCTGCTGGTGTCCATCGGGCAGAAC
CTGGGCGCTCACTGGGGCAGAATAGTTTGGGTCACCACCAACAAGATCGGTTGTGCTGTGAACACCTG
CCGGAAGATGACTGTCTGGGGAGAAGTTTGGGAGAACGCGGTCTACTTTGTCTGCAATTATTCTCCAA
AGGGGAACTGGATTGGAGAAGCCCCCTACAAGAATGGCCGGCCCTGCTCTGAGTGCCCACCCAGCTAT
GGAGGCAGCTGCAGGAACAACTTGTGTTACCGAGAAGAAACCTACACTCCAAAACCTGAAACGGACGA
GATGAATGAGGTGGAAACGGCTCCCATTCCTGAAGAAAACCATGTTTGGCTCCAACCGAGGGTGATGA
GACCCACCAAGCCCAAGAAAACCTCTGCGGTCAACTACATGACCCAAGTCGTCAGATGTGACACCAAG
ATGAAGGACAGGTGCAAAGGGTCCACGTGTAACAGGTACCAGTGCCCAGCAGGCTGCCTGAACCACAA
GGCGAAGATCTTTGGAAGTCTGTTCTATGAAAGCTCGTCTAGCATATGCCGCGCCGCCATCCACTACG
GGATCCTGGATGACAAGGGAGGCCTGGTGGATATCACCAGGAACGGGAAGGTCCCCTTCTTCGTGAAG
TCTGAGAGACACGGCGTGCAGTCCCTCAGCAAATACAAACCTTCCAGCTCATTCATGGTGTCAAAAGT
GAAAGTGCAGGATTTGGACTGCTACACGACCGTTGCTCAGCTGTGCCCGTTTGAAAAGCCAGCAACTC
ACTGCCCAAGAATCCATTGTCCGGCACACTGCAAAGACGAACCTTCCTACTGGGCTCCGGTGTTTGGA
ACCAACATCTATGCAGATACCTCAAGCATCTGCAAGACAGCTGTGCACGCGGGAGTCATCAGCAACGA
GAGTGGGGGTGACGTGGACGTGATGCCCGTGGATAAAAAGAAGACCTACGTGGGCTCGCTCAGGAATG
GAGTTCAGTCTGAAAGCCTGGGGACTCCTCGGGATGGAAAGGCCTTCCGGATCTTTGCTGTCAGGCAG
TGAATTTCCAGCACCAGGGGAGAAGGGGCGTCTTCAGGAGGGCTTCGGGGTTTTGCTTTTATTTTTAT
TTTGTCATTGCGGGGTATATGGAGAGTCAGGAAACTTCCTTTGACTGATGTTCAGTGTCCATCACTTT
GTGGCCTGTGGGTGAGGTGACATCTCATCCCCTCACTGAAGCAACAGCATCCCAAGGTGCTCAGCCGC
ACTCCCTGGTGCCTGATCCTGCTGGGGCCCGGGGGTCTCCATCTGGACGTCCTCTCTCCTTTAGAGAT
CTGAGCTGTCTCTTAAAGGGGACAGTTGCCCAAAATGTTCCTTGCTATGTGTTCTTCTGTTGGTGGAG
GAAGTTGATTTCAACCTCCCTGCCAAAAGAACAAACCATTTGAAGCTCACAATTGTGAAGCATTCACG
GCGTCGGAAGAGGCCTTTTGAGCAAGCGCCAATGAGTTTCAGGAATGAAGTAGAAGGTAGTTATTTAA
AAATAAAAAACACAGTCCGTCCCTACCAATAGAGGAAAATGGTTTTAATGTTTGCTGGTCAGACAGAC
AAATGGGCTAGAGTAAGAGGGCTGCGGGTATGAGAGACCCCGGCTCCGCCCTGGCACGTGTCCTTGCT
GGCGGCCCGCCACAGGCCCCCTTCAATGGCCGCATTCAGGATGGCTCTATACACAGCAGTGCTGGTTT
ATGTAAAGTTCAGCAGTCACTTCA

| | | | |
|---|---|---|---|
| NOV6p, SNP13382488 of | SEQ ID NO: 112 | 453 aa | MW at 50641.5kD |
| CG50391-08, Protein | SNP Pos: 399 | | SNP Change: Ala to Ala |
| Sequence | | | |

MSCVLGGVIPLGLLFLVCGSQGYLLPNVTLLEELLSKYQHNESHSRVRPAIPREDKEEILMLHNKLRG
QVQPQASNMEYMTWDDELEKSAAAWASQCIWEHGPTSLLVSIGQNLGAHWGRIVWVTTNKIGCAVNTC
RKMTVWGEVWENAVYFVCNYSPKGNWIGEAPYKNGRPCSECPPSYGGSCRNNLCYREETYTPKPETDE
MNEVETAPIPEENHVWLQPRVMRPTKPKKTSAVNYMTQVVRCDTKMKDRCKGSTCNRYQCPAGCLNHK
AKIFGSLFYESSSSICRAAIHYGILDDKGGLVDITRNGKVPFFVKSERHGVQSLSKYKPSSSFMVSKV
KVQDLDCYTTVAQLCPFEKPATHCPRIHCPAHCKDEPSYWAPVFGTNIYADTSSICKTAVHAGVISNE
SGGDVDVMPVDKKKTYVGSLENGVQSESLGTPPDGKAFRIFAVRQ

A ClustalW comparison of the above protein sequences yields the following sequence alignment shown in Table 6B.

TABLE 6B
Comparison of the NOV6 protein sequences.

NOV6a ----------------------------------------MSCVLGGVIPLGLLFLVCGS
NOV6b ----------------------------------------MSCVLGGVIPLGLLFLVCGS
NOV6c ----------------------------------------MSCVLGGVIPLGLLFLVCGS
NOV6d ------------------------------------------------------------
NOV6e ----------------------------------------MSCVLGGVIPLGLLFLVCGS
NOV6f ----------------------------------------MSCVLGGVIPLGLLFLVRGS
NOVGg ----------------------------------------MSCVLGGVIPLGLLFLVRGS
NOV6h ----------------------------------------MSCVLGGVIPLGLLFLVRGS
NOV6i ----------------------YLLPNVTLLEELLSKYQHNESHSRVRRAIPREDKEEIL
NOVGj ----------------------------------------MSCVLGGVIPLGLLFLVCGS
NOVGk MSCVLGGVIPLGLLFLVCGSQGYLLPNVTLLEELLSKYQHNESHSRVRRAIPREDKEEIL
NOV6a QGYLLPNVTLLEELLSKYQHNESHSRVRRAIPREDKEEILMLHNKLRGQVQPQAS-----
NOV6b QGYLLPNVTLLEELLSKYQHNESHSRVRRAIPREDKEEILMLHNKLRGQVQPQAS-----
NOV6c QGYLLPNVTLLEELLSKYQHNESHSRVRRAIPREDKEEILMLHNKLRGQVQPQAS-----
NOV6d ------------------------------------------------------M-----
NOV6e QGYLLPNVTLLEELLSKYQHNESHSRVRRAIPREDKEEILMLHNKLRGQVQPQASNMEYM
NOV6f QGYLLPNVTLLEELLSKYQHNESHSRVRRAIPREDKEEILMLHNKLRGQVQPQASNMEYM
NOV6g QGYLLPNVTLLEELLSKYQHNESHSRVRRAIPREDKEEILMLHNKLRGQVQPQAS-----
NOV6h QGYLLPNVTLLEELLSKYQHNESHSRVRRAIPREDKEEILMLHNKLRGQVQPQASNMEYM
NOV6i MLHNKLRGQVQPQASNMEYMTWDDELEKSAAAWASQCIWEHGPTGLLVSIGQNLG-----
NOV6j QGYLLPNVTLLEELLSKYQHNESHSRVRRAIPREDKEEILMLHNKLRGQVQPQASNMEYM
NOV6k MLHNKLRGQVQPQASNMEYMTWDDELEKSAAAWASQCIWEHGPTSLLVSIGQNLG-----
NOV6a ------------------------------------------N--MEYMTWDDELEKSAA
NOV6b -----------------------------------NMEYMYRSPGFHVQSWYDEVKDYTY
NOV6c ------------------------------------------N--MEYMTWDDELEKSAA
NOV6d -----------------------------------TNWGRYRSPGFHVQSWYDEVKDYTY
NOV6e TWDDELEKSAAAWASQCIWEHGPTGLLVSIGQNLGAHWGRYRSPGFHVQSWYDEVKDYTY
NOV6f TWDDELEKSAAAWASQCIWEHGPTGLLVSIGQNLGAHWGRYRSPGFHVQSWYDEVKDYTY
NOV6g ------------------------------------------N--MEYMTWDDELEKSAA
NOVGh TWDDELEKSAAAWASQCIWEHGPTGLLVSIGQNLGAHWGRYRSPGFHVQSWYDEVKDYTY
NOV6i -----------------------------------AHWGRYRSPGFHVQSWYDEVKDYTY
NOV6j TWDDELEKSAAAWASQCIWEHGPTSLLVSIGQNLGAHWGRYRSPGFHVQSWYDEVKDYTY
NOV6k -----------------------------------AHWGRYRSPGFHVQSWYDEVKDYTY
NOV6a AWASQC-----------IWEHG--PTSLLVSIGQNLGAHWGRIV----------------
NOV6b PYPSECNPWCPERCSGPMCTHY---TQIVWATTNKIGCAVNTCRKMTVWGEVWENAVYFV
NOV6c AWASQC-----------IWEHG--PTSLLVSIGQNLGAHWG-------------------
NOV6d PYPSECNPWCPERCSGPMCTHY---TQIVWATTNKIGCAVNTCRKMTVWGEVWENAVYFV
NOV6e PYPSECNPWCPERCSGPMCTHY---TQIVWATTNKIGCAVNTCRKMTVWGEVWENAVYFV
NOV6f PYPSECNPWCPERCSGPMCTHY---TQIVWATTNKIGCAVNTCRKMTVWGEVWENAVYFV
NOV6g AWASQC-----------IWEHG--PTSLLVSIGQNLGAHWGRRYRSPGFHVQSWYDEVKD
NOV6h PYPSECNPWCPERCSGPMCTHYTQVTQIVWATTNKIGCAVNTCRKMTVWGEVWENAVYFV
NOV6i PYPSECNPWCPERCSGPMCTHY---TQIVWATTNKIGCAVNTCRKMTVWGEVWENAVYFV
NOV6j PYPSECNPWCPERCSGPMCTHY---TQIVWATTNKIGCAVNTCRKMTVWGEVWENAVYFV
NOV6k PYPSECNPWCPERCSGPMCTHY---TQIVWATTNKIGCAVNTCRKMTVWGEVWENAVYFV
NOV6a -----------------------------WVTTNKIGCAVNTCRKMTVWGEVWENAVYFV
NOV6b CNYSPK------------------------------------------------------
NOV6c ------------------------------------------------------------
NOV6d CNYSPK------------------------------------------------------
NOV6e CNYSPK------------------------------------------------------
NOV6f CNYSPK------------------------------------------------------
NOV6g YTYPYPSECNPWCPERCSGPMCTHYTQIVWATTNKIGCAVNTCRKMTVWGEVWENAVYFV
NOVGh CNYSPKR-----------------------------------------------------
NOV6i CNYSPK------------------------------------------------------
NOV6j CNYSPK------------------------------------------------------
NOV6k CNYSPK------------------------------------------------------
NOV6a CNYSPKGNWIGEAPYKNGRPCSECPPSYGGSCRNNLCYREETYTPKPETDEMNEVETAPI
NOV6b ------GNWIGEAPYKNGRPCSECPPSYGGSCRNNLCYREETYTPKPETDEMNEVETAPI
NOV6c -----RGNWIGEAPYKNGRPCSECPPSYGGSCRNNLCYREETYTPKPETDEMNEVETAPI
NOV6d ------GNWIGEAPYKNGRPCSECPPSYGGSCRNNLCYREETYTPKPETDEMNEVETAPI
NOV6e ------GNWIGEAPYKNGRPCSQCPPSYGGSCRNNLCYREETYTPKPETDEMNEVETAPI
NOV6f ------GNWIGEAPYKNGRPCSECPPSYGGSCRNNLCYREETYTPKPETDEMNEVETAPI
NOV6g CNYSPKGNWIGEAPYKNGRPCSECPPSYGGSCRNNLCYREETYTPKPETDEMNEVETAPI
NOV6h ------GNWIGEAPYKNGRPCSECPPSYGGSCRNNLCYREETYTPKPETDEMNEVETAPI
NOV6i ------GNWIGEAPYKNGRPCSECPPSYGGSCRNNLCYREETYTPKPETDEMNEVETAPI
NOV6j ------GNWIGEAPYKNGRPCSECPPSYGGSCRNNLCYREETYTPKPETDEMNEVETAPI
NOV6k ------GNWIGEAPYKNGRPCSECPPSYGGSCRNNLCYREETYTPKPETDEMNEVGTAPI
NOV6a PEENHVWLQPRVMRPTKPKXTSAVNYMTQVVRCDTKMKDRCKGSTCNRYQCPAGCLNHKA
NOV6b PEENHVWLQPRVMRPTKPKXTSAVNYMTQVVRCDTKMKDRCKGSTCNRYQCPAGCLNHKA
NOV6c PEENHVWLQPRVMRPTKPKXTSAVNYMTQVVRCDTKMKDRCKGSTCNRYQCPAGCLNHKA
NOV6d PEENHVWLQPRVMRPTKPKXTSAVNYMTQVVRCDTKMKDRCKGSTCNRYQCPAGCLNHKA
NOV6e PEENHVWLQPRVMRPTKPKXTSAVNYMTQVVRCDTKMKDRCKGSTCNRYQCPAGCLNHKA
NOV6f PEENHVWLQPRVMRPTKPKXTSAVNYMTQVVRCDTKMKDRCKGSTCNRYQCPAGCLNHKA
NOV6g PEENHVWLQPRVMRPTKPKXTSAVNYMTQVVRCDTKMKDRCKGSTCNRYQCPAGCLNHKA
NOV6h PEENHVWLQPRVMRPTKPKXTSAVNYMTQVVRCDTKMKDRCKGSTCNRYQCPAGCLNHKA
NOV6i PEENHVWLQPRVMRPTKPKXTSAVNYMTQVVRCDTKMKDRCKGSTCNRYQCPAGCLNHKA
NOV6j PEENHVWLQPRVMRPTKPKXTSAVNYMTQVVRCDTKMKDRCKGSTCNRYQCPAGCLNHKA
NOV6k PEENHVWLQPRVMRPTKPKXTSAVNYMTQVVRCDTKMKDRCKGSTCNRYQCPAGCLNHKA
NOV6a KIFGSLFYESSSSICRAAIHYGILDDKGGLVDITRNGKVPFFVKSERHGVQSLSKYKPSS
NOV6b KIFGTLFYESSSSICRAAIHYGILDDKGGLVDITRNGKVPFFVKSERHGVQSLSKYKPSS
NOV6c KIFGSLFYESSSSICRAAIHYGILDDKGGLVDITRNGKVPFFVKSERHGVQSL-------
NOV6d KIFGTLFYESSSSICRAAIHYGILDDKGGLVDITRNGKVPFFVKSERHGVQSLSKYKPSS
NOV6e KIFGTLFYESSSSICRAAIHYGILDDKGGLVDITRNGKVPFFVKSERHGVQSLSKYKPSS
NOV6f KIFGTLFYESSSSICRAAIHYGILDDKGGLVDITRNGKVPFFVKSERHGVQSLSKYKPSS
NOV6g KIFGSLFYESSSSICRAAIHYGILDDKGGLVDITRNGKVPFFVKSERHGVQSLSKYKPSS
NOV6h KIFGSLFYESSSSICRAAIHYGILDDKGGLVDITRNGKVPFFVKSERHGVQSLSKYKPSS
NOV6i KIFGTLFYESSSSICRAAIHYGILDDKGGLVDITRNGKVPFFVKSERHGVQSLSKYKPSS
NOV6j KIFGSLFYESSSSICRAAIHYGILDDKGGLVDITRNGKVPFFVKSERHGVQSLSKYKPSS
NOV6k KIFGTLFYESSSSICRAAIHYGILDDKGGLVDITRNGKVPFFVKSERHGVQSLSKYKPSS
NOV6a SFMVSKVKVQDLDCYTTVAQLCPFEKPATHCPRIHCPAHCKDEPSYWAPVFGTNIYADTS
NOV6b SFMVSKVKVQDLDCYTTVAQLCPFEKPATHCPRIHCPAHCKDEPSYWAPVFGTNIYADTS
NOV6c --------VQDLDCYTTVAQLCPFEKPATHCPRIHCPAHCKDEPSYWAPVFGTNIYADTS
NOV6d SFMVSKVKVQDLDCYTTVAQLCPFEKPATHCPRIHCPAHCKDEPSYWAPVFGTNIYADTS
NOV6e SFMVSKVKVQDLDCYTTVAQLCPFEKPATHCPRIHCPAHCKDEPSYWAPVFGTNIYADTS
NOV6f SFMVSKVKVQDLDCYTTVAQLCPFEKPATHCPRIHCPAHCKDEPSYWAPVFGTNIYADTS
NOV6g SFMVSKVKVQDLDCYTTVAQLCPFEKPATHCPRIHCPAHCKDEPSYWAPVFGTNIYADTS
NOV6h SFMVSKVKVQDLDCYTTVAQLCPFEKPATHCPRIHCPAHCKDEPSYWAPVFGTNIYADTS
NOV6i SFMVSKVKVQDLDCYTTVAQLCPFEKPATHCPRIHCPAHCKDEPSYWAPVFGTNIYADTS
NOV6j SFMVSKVKVQDLDCYTTVAQLCPFEKPATHCPRIHCPAHCKDEPSYWAPVFGTNIYADTS
NOV6k SFMVSKVKVQDLDCYTTVAQLCPFEKPATHCPRIHCPAHCKDEPSYWAPVFGTNIYADTS
NOV6a SICKTAVHAGVISNESGGDVDVMPVDKKKTYVGSLRNGVQSESLGTPRDGKAFRIFAVRQ
NOV6b SICKTAVHAGVISNESGGDVDVMPVDKKKTYVGSLRNGVQSESLGTPRDGKAFRIFAVRQ
NOV6c SICKTAVHAGVISNESGGDVDVMPVDKKKTYVGSLRNGVQSESLGTPRDGKAFRIFAVRQ
NOV6d SICKTAVHAGVISNESGGDVDVMPVDKKKTYVGSLRNGVQSESLGTPRDGKAFRIFAVRQ
NOV6e SICKTAVHAGVISNESGGDVDVMPVDKKKTYTCPAAARAL--------------------
NOV6f SICKTAVHAGVISNESGGDVDVMPVDKKKTYVGSLRNGVQSESLGTPRDGKAFRIFAVRQ
NOV6g SICKTAVHAGVISNESGGDVDVMPVDKKKTYVGSLRNGVQSESLGTPRDGKAFRIFAVRQ
NOV6h SICKTAVHAGVISNESGGDVDVMPVDKKKTYVGSLRNGVQSESLGTPRDGKAFRIFAVRQ
NOV6i SICKTAVHAGVISNESGGDVDVMPVDKKKTYVGSLRNGVQSESLGTPRDGKAFRIFAVRQ
NOV6j SICKTAVHAGVISNESGGDVDVMPVDKKKTYVGSLRNGVQSESLGTPRDGKAFRIFAVRQ
NOV6k SICKTAVHAGVISNESGGDVDVMPVDKKKTYVGSLRNGVQSESLGTPRDGKAFRIFAVRQ
NOV6a (SEQ ID NO: 82)
NOV6b (SEQ ID NO: 84)
NOV6c (SEQ ID NO: 86)
NOV6d (SEQ ID NO: 88)
NOV6e (SEQ ID NO: 90)
NOV6f (SEQ ID NO: 92)
NOV6g (SEQ ID NO: 94)
NOV6h (SEQ ID NO: 96)
NOV6i (SEQ ID NO: 98)
NOV6j (SEQ ID NO: 100)
NOV6k (SEQ ID NO: 102)

Further analysis of the NOV6a protein yielded the following properties shown in Table 6C.

TABLE 6C
Protein Sequence Properties NOV6a

| | |
|---|---|
| SignalP analysis: | Cleavage site between residues 23 and 24 |
| PSORT II analysis: | |

| PSG: | a new signal peptide prediction method | |
|---|---|---|
| | N-region: | length 0; pos. chg 0; neg. chg 0 |
| | H-region: | length 31; peak value 10.73 |
| | PSG score: | 6.33 |

| | |
|---|---|
| GvH: | von Heijne's method for signal seq. recognition |
| | GvH score (threshold: −2.1): −0.72 |
| | possible cleavage site: between 22 and 23 |

>>> Seems to have a cleavable signal peptide (1 to 22)

| ALOM: | Klein et al's method for TM region allocation | |
|---|---|---|
| | Init position for calculation: 23 | |
| | Tentative number of TMS(s) for the threshold 0.5: 0 | |
| | number of TMS(s) . . . fixed | |
| | PERIPHERAL | Likelihood = 7.37 (at 106) |

| | ALOM score: 7.37 (number of TMSs: 0) | | | |
|---|---|---|---|---|
| MTOP: | Prediction of membrane topology (Hartmann et al.) | | | |
| | Center position for calculation: 11 | | | |
| | Charge difference: −1.0 C(0.0)-N(1.0) | | | |
| | N >= C: N-terminal side will be inside | | | |
| MITDISC: | discrimination of mitochondrial targeting seq | | | |
| | R content: | 0 | Hyd Moment (75): | 0.40 |
| | Hyd Moment (95): | 3.00 | G content: | 5 |
| | D/E content: | 1 | S/T content: | 3 |

| | |
|---|---|
| | Score: −7.63 |
| Gavel: | prediction of cleavage sites for mitochondrial preseq |
| | cleavage site motif not found |

NUCDISC: discrimination of nuclear localization signals

| | |
|---|---|
| | pat4: KPKK (4) at 230 |
| | pat7: PTKPKKT (4) at 228 |
| | pat7: PVDKKKT (4) at 417 |
| | bipartite: none |
| | content of basic residues: 11.9% |
| | NLS Score: 0.47 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear |
| | discrimination |
| | Prediction: nuclear |
| | Reliability: 55.5 |
| COIL: | Lupas's algorithm to detect coiled-coil regions |
| | total: 0 residues |

Final Results (k = 9/23):

```
52.2%: nuclear
21.7%: extracellular, including cell wall
17.4%: mitochondrial
  4.3%: vacuolar
  4.3%: cytoplasmic
```

>> prediction for CG50391-08 is nuc (k = 23)

A search of the NOV6a protein against the Geneseq database, a proprietary database that contains sequences published in patents and patent publication, yielded several homologous proteins shown in Table 6D.

TABLE 6D
Geneseq Results for NOV6a

| | | NOV6a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAU83619 | Human PRO protein, Seq ID No 56 - | 1 . . . 453 | 452/497 (90%) | 0.0 |
| | Homo sapiens, 497 aa. | 1 . . . 497 | 452/497 (90%) | |
| | [WO200208288-A2, 31-JAN-2002] | | | |
| AAO21721 | Human secreted protein SEQ ID No 63 - | 1 . . . 453 | 452/497 (90%) | 0.0 |
| | Homo sapiens, 497 aa. | 1 . . . 497 | 452/497 (90%) | |
| | [WO200238602-A2, 16-MAY-2002] | | | |
| AAB74446 | Human protease-inhibitor like protein - | 1 . . . 453 | 452/497 (90%) | 0.0 |
| | Homo sapiens, 497 aa. | 1 . . . 497 | 452/497 (90%) | |
| | [WO200121651-A2, 29-MAR-2001] | | | |
| AAE10618 | Human novel trypsin inhibitor-like | 1 . . . 453 | 451/498 (90%) | 0.0 |
| | protein, NOV-4d - Homo sapiens, 498 | 1 . . . 498 | 451/498 (90%) | |
| | aa. [WO200162928-A2, 30-AUG-2001] | | | |
| AAE10616 | Human novel trypsin inhibitor-like | 1 . . . 453 | 450/497 (90%) | 0.0 |
| | protein, NOV-4b - Homo sapiens, 497 | 1 . . . 497 | 450/497 (90%) | |
| | aa. [WO200162928-A2, 30-AUG-2001] | | | |

In a BLAST search of public sequence databases, the NOV6a protein was found to have homology to the proteins shown in the BLASTP data in Table 6E.

TABLE 6E
Public BLASTP Results for NOV6a

| | | NOV6a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9H0B8 | Hypothetical protein - Homo | 1 . . . 453 | 451/497 (90%) | 0.0 |
| | sapiens (Human), 497 aa. | 1 . . . 497 | 452/497 (90%) | |
| CAC69820 | Sequence 20 from Patent | 1 . . . 453 | 451/498 (90%) | 0.0 |
| | WO0162928 - Homo sapiens | 1 . . . 498 | 451/498 (90%) | |
| | (Human), 498 aa. | | | |
| CAC69818 | Sequence 16 from Patent | 1 . . . 453 | 450/497 (90%) | 0.0 |
| | WO0162928 - Homo sapiens | 1 . . . 497 | 450/497 (90%) | |
| | (Human), 497 aa. | | | |
| CAC69821 | Sequence 22 from Patent | 1 . . . 453 | 450/501 (89%) | 0.0 |
| | WO0162928 - Homo sapiens | 1 . . . 501 | 450/501 (89%) | |
| | (Human), 501 aa. | | | |
| CAC69817 | Sequence 14 from Patent | 81 . . . 453 | 339/373 (90%) | 0.0 |
| | WO0162928 - Homo sapiens | 16 . . . 383 | 348/373 (92%) | |
| | (Human), 383 aa. | | | |

PFam analysis predicts that the NOV6a protein contains the domains shown in the Table 6F.

TABLE 6F
Domain Analysis of NOV6a

| | | Identities/ | |
|---|---|---|---|
| | | Similarities | |
| Pfam | NOV6a Match | for the | Expect |
| Domain | Region | Matched Region | Value |
| SCP | 60 . . . 155 | 45/167 (27%) | 7.4e−23 |
| | | 76/167 (46%) | |
| LCCL | 244 . . . 335 | 47/104 (45%) | 6.1e−49 |
| | | 84/104 (81%) | |
| LCCL | 345 . . . 444 | 55/104 (53%) | 1.6e−51 |
| | | 89/104 (86%) | |

### Example 7

The NOV7 clone was analyzed, and the nucleotide and encoded polypeptide sequences are shown in Table 7A.

TABLE 7A
NOV7 Sequence Analysis

| | | |
|---|---|---|
| NOV7a, CG50426-17 | SEQ ID NO: 113 | 8160 bp |
| DNA Sequence | ORF Start: ATG at 122 | ORF Stop: TAA at 8108 |

GGGAGTTGGAACCAGCATTAGATGAGTTGACAAAAATGCAGTTTCAGGTGGCAAAGTGACAAGTGCCA
AAACTCAGGCCTGACTTTTCTGAAAACATCAGCATTCTGCCATATCTGGAATAATGGATGTAAAGGAC
CGGCGACACCGCTCTTTGACCAGAGGACGCTGTGGCAAAGAGTGTCGCTACACAAGCTCCTCTCTGGA
CAGTGAGGACTGCCGGGTGCCCACACAGAAATCCTACAGCTCCAGTGAGACTCTGAAGGCCTATGACC
ATGACAGCAGGATGCACTATGGAAACCGAGTCACAGACCTCATCCACCGGGAGTCAGATGAGTTTCCT
AGACAAGGAACCAACTTCACCCTTGCCGAACTGGGCATCTGTGAGCCCTCCCCACACCGAAGCGGCTA
CTGCTCCGACATGGGGATCCTTCACCAGGGCTACTCCCTTAGCACAGGGTCTGACGCCGACTCCGACA
CCGAGGGAGGGATGTCTCCAGAACACGCCATCAGACTGTGGGGCAGAGGGATAAAATCCAGGCGCAGT
TCCGGCCTGTCCAGTCGTGAAAACTCGGCCCTTACCCTGACTGACTCTGACAACGAAAACAAATCAGA
TGATGAGAACGGTCGTCCCATTCCACCTACATCCTCGCCTAGTCTCCTCCCATCTGCTCAGCTGCCTA
GCTCCCATAATCCTCCACCAGTTAGCTGCCAGATGCCATTGCTAGACAGCAACACCTCCCATCAAATC
ATGGACACCAACCCTGATGAGGAATTCTCCCCCAATTCATACCTGCTCAGAGCATGCTCAGGGCCCCA
GCAAGCCTCCAGCAGTGGCCCTCCGAACCACCACAGCCAGTCGACTCTGAGGCCCCCTCTCCCACCCC
CTCACAACCACACGCTGTCCCATCACCACTCGTCCGCCAACTCCCTCAACAGGAACTCACTGACCAAT
CGGCGGAGTCAGATCCACGCCCCGGCCCCAGCGCCCAATGACCTGGCCACCACACCAGAGTCCGTTCA
GCTTCAGGACAGCTGGGTGCTAAACAGCAACGTGCCACTGGAGACCCGGCACTTCCTCTTCAAGACCT
CCTCGGGGAGCACACCCTTGTTCAGCAGCTCTTCCCCGGGATACCCTTTGACCTCAGGAACGGTTTAC
ACGCCCCCGCCCCGCCTGCTGCCCAGGAATACTTTCTCCAGGAAGGCTTTCAAGCTGAAGAAGCCCTC
CAAATACTGCAGCTGGAAATGTGCTGCCCTCTCCGCCATTGCCGCGGCCCTCCTCTTGGCTATTTTGC
TGGCGTATTTCATAGTGCCCTGGTCGTTGAAAAACAGCAGCATAGACAGTGGTGAAGCAGAAGTTGGT
CGGCGGGTAACACAAGAAGTCCCACCAGGGGTGTTTTGGAGGTCACAAATTCACATCAGTCAGCCCCA
GTTCTTAAAGTTCAACATCTCCCTCGGGAAGGACGCTCTCTTTGGTGTTTACATAAGAAGAGGACTTC
CACCATCTCATGCCCAGTATGACTTCATGGAACGTCTGGACGGGAAGGAGAAGTGGAGTGTGGTTGAG
TCTCCCAGGGAACGCCGGAGCATACAGACCTTGGTTCAGAATGAAGCCGTGTTTGTGCAGTACCTGGA
TGTGGGCCTGTGGCATCTGGCCTTCTACAATGATGGAAAAGACAAAGAGATGGTTTCCTTCAATACTG
TTGTCCTAGATTCAGTGCAGGACTGTCCACGTAACTGCCATGGGAATGGTGAATGTGTGTCCGGGGTG
TGTCACTGTTTCCCAGGATTTCTAGGAGCAGACTGTGCTAAAGCTGCCTGCCCTGTCCTGTGCAGTGG
GAATGGACAATATTCTAAAGGGACGTGCCAGTGCTACAGCGGCTGGAAAGGTGCAGAGTGCGACGTGC
CCATGAATCAGTGCATCGATCCTTCCTGCGGGGGCCACGGCTCCTGCATTGATGGGAACTGTGTCTGC
TCTGCTGGCTACAAAGGCGAGCACTGTGAGGAAGTTGATTGCTTGGATCCCACCTGCTCCAGCCACGG
AGTCTGTGTGAATGGAGAATGCCTGTGCAGCCCTGGCTGGGGTGGTCTGAACTGTGAGCTGGCGAGGG
TCCAGTGCCCAGACCAGTGCAGTGGGCATGGCACGTACCTGCCTGACACGGGCCTCTGCAGCTGCGAT
CCCAACTGGATGGGTCCCGACTGCTCTGTTGATGGCTGCCCTGACTTGTGCAACGGTAACGGGAGATG
CACACTGGGTCAGAACAGCTGGCAGTGTGTCTGCCAGACCGGCTGGAGAGGGCCCGGATGCAACGTTG
CCATGGAAACTTCCTGTGCTGATAACAAGGATAATGAGGGAGATGGCCTGGTGGATTGTTTGGACCCT
GACTGCTGCCTGCAGTCAGCCTGTCAGAACAGCCTGCTCTGCCGGGGGTCCCGGGACCCACTGGACAT
CATTCAGCAGGGCCAGACGGATTGGCCCGCAGTGAAGTCCTTCTATGACCGTATCAAGCTCTTGGCAG
GCAAGGATAGCACCCACATCATTCCTGGAGAGAACCCTTTCAACAGCAGCTTGGTTTCTCTCATCCGA
GGCCAAGTAGTAACTACAGATGGAACTCCCCTGGTCGGTGTGAACGTGTCTTTTGTCAAGTACCCAAA
ATACGGCTACACCATCACCCGCCAGGATGGCACGTTCGACCTGATCGCAAATGGAGGTGCTTCCTTGA
CTCTACACTTTGAGCGAGCCCCGTTCATGAGCCAGGAGCGCACTGTGTGGCTGCCGTGGAACAGCTTT
TACGCCATGGACACCCTGGTGATGAAGACCGAGGAGAACTCCATCCCCAGCTGTGACCTCAGTGGCTT
TGTCCGGCCTGATCCAATCATCATCTCCTCCCCACTGTCCACCTTCTTTAGTGCTGCCCCTGGGCAGA
ATCCCATCGTGCCTGAGACCCAGGTTCTTCATGAAGAAATCGAGCTCCCTGGTTCCAATGTGAAACTT
CGCTATCTGAGCTCTAGAACTGCAGGGTACAAGTCACTGCTGAAGATCACCATGACCCAGTCCACAGT
GCCCCTGAACCTCATTAGGGTTCACCTGATGGTGGCTGTCGAGGGGCATCTCTTCCAGAAGTCATTCC
AGGCTTCTCCCAACCTGGCCTCCACCTTCATCTGGGACAAGACAGATGCGTATGGCCAAAGGGTGTAT
GGACTCTCAGATGCTGTTGTGTCTGTCGGGTTTGAATATGAGACCTGTCCCAGTCTAATTCTCTGGGA
GAAAAGGACAGCCCTCCTTCAGGGATTCGAGCTGGACCCCTCCAACCTCGGTGGCTGGTCCCTAGACA
AACACCACATCCTCAATGTTAAAAGTGGAATCCTACACAAAGGCACTGGGGAAAACCAGTTCCTGACC
CAGCAGCCTGCCATCATCACCAGCATCATGGGCAATGGTCGCCGCCGGAGCATTTCCTGTCCCAGCTG
CAACGGCCTTGCTGAAGGCAACAAGCTGCTGGCCCCAGTGGCTCTGGCTGTTGGAATCGATGGGAGCC
TCTATGTGGGTGACTTCAATTACATCCGACGCATCTTTCCCTCTCGAAATGTGACCAGCATCTTGGAG
TTACGAAATAAAGAGTTTAAACATAGCAACAACCCAGCACACAAGTACTACTTGGCAGTGGACCCCGT
GTCCGGCTCGCTCTACGTGTCCGACACCAACAGCAGGAGAATCTACCGCGTCAAGTCTCTGAGTGGAA
CCAAAGACCTGGCTGGGAATTCGGAAGTTGTGGCAGGGACGGGAGAGCAGTGTCTACCCTTTGATGAA
GCCCGCTGCGGGGATGGAGGGAAGGCCATAGATGCAACCCTGATGAGCCCGAGAGGTATTGCAGTAGA
CAAGAATGGGCTCATGTACTTTGTCGATGCCACCATGATCCGGAAGGTTGACCAGAATGGAATCATCT
CCACCCTGCTGGGCTCCAATGACCTCACTGCCGTCCGGCCGCTGAGCTGTGATTCCAGCATGGATGTA
GCCCAGGTTCGTCTGGAGTGGCCAACAGACCTTGCTGTCAATCCCATGGATAACTCCTTGTATGTTCT
AGAGAACAATGTCATCCTTCGAATCACCGAGAACCACCAAGTCAGCATCATTGCGGGACGCCCCATGC
ACTGCCAAGTTCCTGGCATTGACTACTCACTCAGCAAACTAGCCATTCACTCTGCCCTGGAGTCAGCC
AGTGCCATTGCCATTTCTCACACTGGGGTCCTCTACATCACTGAGACAGATGAGAAGAAGATTAACCG
TCTACGCCAGGTAACAACCAACGGGGAGATCTGCCTTTTAGCTGGGGCAGCCTCGGACTGCGACTGCA
AAAACGATGTCAATTGCAACTGCTATTCAGGAGATGATGCCTACGCGACTGATGCCATCTTGAATTCC
CCATCATCCTTAGCTGTAGCTCCAGATGGTACCATTTACATTGCAGACCTTGGAAATATTCGGATCAG
GGCGGTCAGCAAGAACAAGCCTGTTCTTAATGCCTTCAACCAGTATGAGGCTGCATCCCCCGGAGAGC
AGGAGTTATATGTTTTCAACGCTGATGGCATCCACCAATACACTGTGAGCCTGGTGACAGGGGAGTAC
TTGTACAATTTCACATATAGTACTGACAATGATGTCACTGAATTGATTGACAATAATGGGAATTCCCT
GAAGATCCGTCGGGACAGCAGTGGCATGCCCCGTCACCTGCTCATGCCTGACAACCAGATCATCACCC
TCACCGTGGGCACCAATGGAGGCCTCAAAGTCGTGTCCACACAGAACCTGGAGCTTGGTCTCATGACC
TATGATGGCAACACTGGGCTCCTGGCCACCAAGAGCGATGAAACAGGATGGACGACTTTCTATGACTA
TGACCACGAAGGCCGCCTGACCAACGTGACGCGCCCCACGGGGGTGGTAACCAGTCTGCACCGGGAAA
TGGAGAAATCTATTACCATTGACATTGAGAACTCCAACCGTGATGATGACGTCACTGTCATCACCAAC
CTCTCTTCAGTAGAGGCCTCCTACACAGTGGTACAAGATCAAGTTCGGAACAGCTACCAGCTCTGTAA
TAATGGTACCCTGAGGGTGATGTATGCTAATGGGATGGGTATCAGCTTCCACAGCGAGCCCCATGTCC
TAGCGGGCACCATCACCCCCACCATTGGACGCTGCAACATCTCCCTGCCTATGGAGAATGGCTTAAAC
TCCATTGAGTGGCGCCTAAGAAAGGAACAGATTAAAGGCAAAGTCACCATCTTTGGCAGGAAGCTCCG
GGTCCATGGAAGAAATCTCTTGTCCATTGACTATGATCGAAATATTCGGACTGAAAAGATCTATGATG
ACCACCGGAAGTTCACCCTGAGGATCATTTATGACCAGGTGGGCCGCCCCTTCCTCTGGCTGCCCAGC
AGCGGGCTGGCAGCTGTCAACGTGTCATACTTCTTCAATGGGCGCCTGGCTGGGCTTCAGCGTGGGGC
CATGAGCGAGAGGACAGACATCGACAAGCAAGGCCGCATCGTGTCCCGCATGTTCGCTGACGGGAAAG
TGTGGAGCTACTCCTACCTTGACAAGTCCATGGTCCTCCTGCTTCAGAGCCAACGTCAGTATATATTT
GAGTATGACTCCTCTGACCGCCTCCTTGCCGTCACCATGCCCAGCGTGGCCCGGCACAGCATGTCCAC
ACACACCTCCATCGGCTACATCCGTAATATTTACAACCCGCCTGAAAGCAATGCTTCGGTCATCTTTG
ACTACAGTGATGACGGCCGCATCCTGAAGACCTCCTTTTTGGGCACCGGACGCCAGGTGTTCTACAAG
TATGGGAAACTCTCCAAGTTATCAGAGATTGTCTACGACAGTACCGCCGTCACCTTCGGGTATGACGA
GACCACTGGTGTCTTGAAGATGGTCAACCTCCAAAGTGGGGGCTTCTCCTGCACCATCAGGTACCGGA
AGATTGGCCCCCTGGTGGACAAGCAGATCTACAGGTTCTCCGAGGAAGGCATGGTCAATGCCAGGTTT
GACTACACCTATCATGACAACAGCTTCCGCATCGCAAGCATCAAGCCCGTCATAAGTGAGACTCCCCT
CCCCGTTGACCTCTACCGCTATGATGAGATTTCTGGCAAGGTGGAACACTTTGGTAAGTTTGGAGTCA
TCTATTATGACATCAACCAGATCATCACCACTGCCGTGATGACCCTCAGCAAACACTTCGACACCCAT
GGGCGGATCAAGGAGGTCCAGTATGAGATGTTCCGGTCCCTCATGTACTGGATGACGGTGCAATATGA
CAGCATGGGCAGGGTGATCAAGAGGGAGCTAAAACTGGGGCCCTATGCCAATACCACGAAGTACACCT
ATGACTACGATGGGGACGGGCAGCTCCAGAGCGTGGCCGTCAATGACCGCCCGACCTGGCGCTACAGC
TATGACCTTAATGGGAATCTCCACTTACTGAACCCAGGCAACAGTGTGCGCCTCATGCCCTTGCGCTA
TGACCTCCGGGATCGGATAACCAGACTCGGGGATGTGCAGTACAAAATTGACGACGATGGCTATCTGT
GCCAGAGAGGGTCTGACATCTTCGAATACAATTCCAAGGGCCTCCTAACAAGAGCCTACAACAAGGCC
AGCGGGTGGAGTGTCCAGTACCGCTATGATGGCGTAGGACGGCGGGCTTCCTACAAGACCAACCTGGG
CCACCACCTGCAGTACTTCTACTCTGACCTCCACAACCCGACGCGCATCACCCATGTCTACAATCACT
CCAACTCGGAGATTACCTCACTGTACTACGACCTCCAGGGCCACCTCTTTGCCATGGAGAGCAGCAGT
GGGGAGGAGTACTATGTTGCCTCTGATAACACAGGGACTCCTCTGGCTGTGTTCAGCATCAACGGCCT
CATGATCAAACAGCTGCAGTACACGGCCTATGGGGAGATTTATTATGACTCCAACCCCGACTTCCAGA
TGGTCATTGGCTTCCATGGGGGACTCTATGACCCCCTGACCAAGCTGGTCCACTTCACTCAGCGTGAT
TATGATGTGCTGGCAGGACGATGGACCTCCCCAGACTATACCATGTGGAAAAACGTGGGCAAGGAGCC
GGCCCCCTTTAACCTGTATATGTTCAAGAGCAACAATCCTCTCAGCAGTGAGCTAGATTTGAAGAACT
ACGTGACAGATGTGAAAAGCTGGCTTGTGATGTTTGGATTTCAGCTTAGCAACATCATTCCTGGCTTC
CCGAGAGCCAAAATGTATTTCGTGCCTCCTCCCTATGAATTGTCAGAGAGTCAAGCAAGTGAGAATGG
ACAGCTCATTACAGGTGTCCAACAGACAACAGAGAGACATAACCAGGCCTTCATGGCTCTGGAAGGAC
AGGTCATTACTAAAAAGCTCCACGCCAGCATCCGAGAGAAAGCAGGTCACTGGTTTGCCACCACCACG
CCCATCATTGGCAAAGGCATCATGTTTGCCATCAAAGAAGGGCGGGTGACCACGGGCGTGTCCAGCAT
CGCCAGCGAAGATAGCCGCAAGGTGGCATCTGTGCTGAACAACGCCTACTACCTGGACAAGATGCACT
ACAGCATCGAGGGCAAGGACACCCACTACTTTGTGAAGATTGGCTCAGCCGATGGCGACCTGGTCACA
CTAGGCACCACCATCGGCCGCAAGGTGCTAGAGAGCGGGGTGAACGTGACCGTGTCCCAGCCCACGCT
GCTGGTCAACGGCAGGACTCGAAGGTTCACGAACATTGAGTTCCAGTACTCCACGCTGCTGCTCAGCA
TCCGCTATGGCCTCACCCCCGACACCCTGGACGAAGAGAAGGCCCGCGTCCTGGACCAGGCGAGACAG
AGGGCCCTGGGCACGGCCTGGGCCAAGGAGCAGCAGAAAGCCAGGGACGGGAGAGAGGGGAGCCGCCT
GTGGACTGAGGGCGAGAAGCAGCAGCTTCTGAGCACCGGGCGCGTGCAAGGGTACGAGGGATATTACG
TGCTTCCCGTGGAGCAATACCCAGAGCTTGCAGACAGTAGCAGCAACATCCAGTTTTTAAGACAGAAT
GAGATGGGAAAGAGGTAACAAAATAATCTGCTGCCATTCCTTGTCTGAATGGCTCAGCAGGAGTAACT

| | | | |
|---|---|---|---|
| NOV7a, CG50426-17 | SEQ ID NO: 114 | 2662 aa | MW at 295881.9kD |
| Protein Sequence | | | |

MDVKDRRHRSLTRGRCGKECRYTSSSLDSEDCRVPTQKSYSSSETLKAYDHDSRMHYGNRVTDLIHRE
SDEFPRQGTNFTLAELGICEPSPHRSGYCSDMGILHQGYSLSTGSDADSDTEGGMSPEHAIRLWGRGI
KSRRSSGLSSRENSALTLTDSDNENKSDDENGRPIPPTSSPSLLPSAQLPSSHNPPPVSCQMPLLDSN
TSHQIMDTNPDEEFSPNSYLLRACSGPQQASSSGPPNHHSQSTLRPPLPPPHNHTLSHHHSSANSLNR
NSLTNRRSQIHAPAPAPNDLATTPESVQLQDSWVLNSNVPLETRHFLFKTSSGSTPLFSSSSPGYPLT
SGTVYTPPPRLLPRNTFSRKAFKLKKPSKYCSWKCAALSAIAAALLLAILLAYFIVPWSLKNSSIDSG
EAEVGRRVTQEVPPGVFWRSQIHISQPQFLKFNISLGKDALFGVYIRRGLPPSHAQYDFMERLDGKEK
WSVVESPRERRSIQTLVQNEAVFVQYLDVGLWHLAFYNDGKDKEMVSFNTVVLDSVQDCPRNCHGNGE
CVSGVCHCFPGFLGADCAKAACPVLCSGNGQYSKGTCQCYSGWKGAECDVPMNQCIDPSCGGHGSCID
GNCVCSAGYKGEHCEEVDCLDPTCSSHGVCVNGECLCSPGWGGLNCELARVQCPDQCSGHGTYLPDTG
LCSCDPNWMGPDCSVDGCPDLCNGNGRCTLGQNSWQCVCQTGWRGPGCNVAMETSCADNKDNEGDGLV
DCLDPDCCLQSACQNSLLCRGSRDPLDIIQQGQTDWPAVKSFYDRIKLLAGKDSTHIIPGENPFNSSL
VSLIRGQVVTTDGTPLVGVNVSFVKYPKYGYTITRQDGTFDLIANGGASLTLHFERAPFMSQERTVWL
PWNSFYAMDTLVMKTEENSIPSCDLSGFVRPDPIIISSPLSTFFSAAPGQNPIVPETQVLHEEIELPG
SNVKLRYLSSRTAGYKSLLKITMTQSTVPLNLIRVHLMVAVEGHLFQKSFQASPNLASTFIWDKTDAY
GQRVYGLSDAVVSVGFEYETCPSLILWEKRTALLQGFELDPSNLGGWSLDKHHILNVKSGILHKGTGE
NQFLTQQPAIITSIMGNGRRRSISCPSCNGLAEGNKLLAPVALAVGIDGSLYVGDFNYIRRIFPSRNV
TSILELRNKEFKHSNNPAHKYYLAVDPVSGSLYVSDTNSRRIYRVKSLSGTKDLAGNSEVVAGTGEQC
LPFDEARCGDGGKAIDATLMSPRGIAVDKNGLMYFVDATMIRKVDQNGIISTLLGSNDLTAVRPLSCD
SSMDVAQVRLEWPTDLAVNPMDNSLYVLENNVILRITENHQVSIIAGRPMHCQVPGIDYSLSKLAIHS
ALESASAIAISHTGVLYITETDEKKINRLRQVTTNGEICLLAGAASDCDCKIWVNCNCYSGDDAYATD
AILNSPSSLAVAPDGTIYIADLGNIRIEAVSKNKPVLNAFNQYEAASPGEQELYVFNADGIHQYTVSL
VTGEYLYNFTYSTDNDVTELIDNNGNSLKIRRDSSGMPRHLLMPDNQIITLTVGTNGGLKVVSTQNLE
LGLMTYDGNTGLLATKSDETGWTTFYDYDHEGRLTNVTRPTGVVTSLHREMEKSITIDIENSNRDDDV
TVITNLSSVEASYTVVQDQVRNSYQLCNNGTLRVMYANGMGISFHSEPHVLAGTITPTIGRCNISLPM
ENGLNSIEWRLRKEQIKGKVTIFGRKLRVHGRNLLSIDYDRNIRTEKIYDDHRKFTLRIIYDQVGRPF
LWLPSSGLAAVNVSYFFNGRLAGLQRGAMSERTDIDKQGRIVSRMFADGKVWSYSYLDKSMVLLLQSQ
RQYIFEYDSSDRLLAVTMPSVARHSMSTHTSIGYIRNIYNPPESNASVIFDYSDDGRILKTSFLGTGR
QVFYKYGKLSKLSEIVYDSTAVTFGYDETTGVLKMVNLQSGGFSCTIRYRKIGPLVDKQIYRFSEEGM
VNARFDYTYHDNSFRIASIKPVISETPLPVDLYRYDEISGKVEHFGKFGVIYYDINQIITTAVMTLSK
HFDTHGRIKEVQYEMFRSLMYWMTVQYDSMGRVIKRELKLGPYANTTKYTYDYDGDGQLQSVAVNDRP
TWRYSYDLNGNLHLLNPGNSVRLMPLRYDLRDRITRLGDVQYKIDDDGYLCQRGSDIFEYNSKGLLTR
AYNKASGWSVQYRYDGVGRPASYKTNLGHHLQYFYSDLHNPTRITHVYNHSNSEITSLYYDLQGHLFA
MESSSGEEYYVASDNTGTPLAVFSINGLMIKQLQYTAYGEIYYDSNPDFQMVIGFHGGLYDPLTKLVH
FTQRDYDVLAGRWTSPDYTMWKNVGKEPAPFNLYMFKSNNPLSSELDLKNYVTDVKSWLVMFGFQLSN
IIPGFPRAKMYFVPPPYELSESQASENGQLITGVQQTTERHNQAFMALEGQVITKKLHASIREKAGHW
FATTTPIIGKGIMFAIKEGRVTTGVSSIASEDSRKVASVLNNAYYLDKMHYSIEGKDTHYFVKIGSAD
GDLVTLGTTIGRKVLESGVNVTVSQPTLLVNGRTRRFTNIEFQYSTLLLSIRYGLTPDTLDEEKARVL
DQARQRALGTAWAKEQQKARDGREGSRLWTEGEKQQLLSTGRVQGYEGYYVLPVEQYPELADSSSNIQ
FLRQNEMGKR

| | | |
|---|---|---|
| NOV7b, CG50426-21 | SEQ ID NO: 115 | 9852 bp |
| DNA Sequence | ORF Start: ATG at 210 | ORF Stop: TAA at 8505 |

CCAGCATTAGATGAGTTGACAAAAATGCAGTTTCAGCTCTGAAGGTCTGAAAGATTCTGCTGCAACTA
AAGCTCTGAAGATTCTGCTACAACTATGACATCCATTTTCTCCCACTTCAGACAGGATGAATACAAGG
TGGCAAAGTGACAAGTGCCAAAACTCAGGCCTGACTTTCCTGAAAACATCAGCATTCTGCCATATCTG
GAATAATGGATGTAAAGGACCGGCGACACCGCTCTTTGACCAGAGGACGCTGTGGCAAAGAGTGTCGC
TACACAAGCTCCTCTCTGGACAGTGAGGACTGCCGGGTGCCCACACAGAAATCCTACAGCTCCAGTGA
GACTCTGAAGGCCTATGACCATGACAGCAGGATGCACTATGGAAACCGAGTCACAGACCTCATCCACC
GGGAGTCAGATGAGTTTCCTAGACAAGGAACCAACTTCACCCTTGCCGAACTGGGCATCTGTGAGCCC
TCCCCACACCGAAGCGGCTACTGCTCCGACATGGGGATCCTTCACCAGGGCTACTCCCTTAGCACAGG
GTCTGACGCCGACTCCGACACCGAGGGAGGGATGTCTCCAGAACACGCCATCAGACTGTGGGGCAGAG
GGATAAAATCCAGGCGCAGTTCCGGCCTGTCCAGTCGTGAAAACTCGGCCCTTACCCTGACTGACTCT
GACAACGAAAACAAATCAGATGATGAGAACGGTCGTCCCATTCCACCTACATCCTCGCCTAGTCTCCT
CCCATCTGCTCAGCTGCCTAGCTCCCATAATCCTCCACCAGTTAGCTGCCAGATGCCATTGCTAGACA
GCAACACCTCCCATCAAATCATGGACACCAACCCTGATGAGGAATTCTCCCCCAATTCATACCTGCTC
AGAGCATGCTCAGGGCCCCAGCAAGCCTCCAGCAGTGGCCCTCCGAACCACCACAGCCAGTCGACTCT
GAGGCCCCCTCTCCCACCCCCTCACAACCACACGCTGTCCCATCACCACTCGTCCGCCAACTCCCTCA
ACAGGAACTCACTGACCAATCGGCGGAGTCAGATCCACGCCCCGGCCCCAGCGCCCAATGACCTGGCC
ACCACACCAGAGTCCGTTCAGCTTCAGGACAGCTGGGTGCTAAACAGCAACGTGCCACTGGAGACCCG
GCACTTCCTCTTCAAGACCTCCTCGGGGAGCACACCCTTGTTCAGCAGCTCTTCCCCGGGATACCCTT
TGACCTCAGGAACGGTTTACACGCCCCCGCCCCGCCTGCTGCCCAGGAATACTTTCTCCAGGAAGGCT
TTCAAGCTGAAGAAGCCCTCCAAATACTGCAGCTGGAAATGTGCTGCCCTCTCCGCCATTGCCGCGGC
CCTCCTCTTGGCTATTTTGCTGGCGTATTTCATAGCAATGCATCTGCTCGGACTCAATTGGCAACTCC
AGCCTGCAGATGGGCACACCTTTAACAATGGGATAAGGACCGGCTTACCAGGAAACGATGATGTGGCA
ACAATGCCATCTGGAGGCAAAGTGCCCTGGTCGTTGAAAAACAGCAGCATAGACAGTGGTGAAGCAGA
AGTTGGTCGGCGGGTAACACAAGAAGTCCCACCAGGGGTGTTTTGGAGGTCACAAATTCACATCAGTC
AGCCCCAGTTCTTAAAGTTCAACATCTCCCTCGGGAAGGACGCTCTCTTTGGTGTTTACATAAGAAGA
GGACTTCCACCATCTCATGCCCAGTATGACTTCATGGAACGTCTGGACGGGAAGGAGAAGTGGAGTGT
GGTTGAGTCTCCCAGGGAACGCCGGAGCATACAGACCTTGGTTCAGAATGAAGCCGTGTTTGTGCAGT
ACCTGGATGTGGGCCTGTGGCATCTGGCCTTCTACAATGATGGAAAAGACAAAGAGATGGTTTCCTTC
AATACTGTTGTCCTAGATTCAGTGCAGGACTGTCCACGTAACTGCCATGGGAATGGTGAATGTGTGTC
CGGGGTGTGTCACTGTTTCCCAGGATTTCTAGGAGCAGACTGTGCTAAAGCTGCCTGCCCTGTCCTGT
GCAGTGGGAATGGACAATATTCTAAAGGGACGTGCCAGTGCTACAGCGGCTGGAAAGGTGCAGAGTGC
GACGTGCCCATGAATCAGTGCATCGATCCTTCCTGCGGGGGCCACGGCTCCTGCATTGATGGGAACTG
TGTCTGCTCTGCTGGCTACAAAGGCGAGCACTGTGAGGAAGTTGATTGCTTGGATCCCACCTGCTCCA
GCCACGGAGTCTGTGTGAATGGAGAATGCCTGTGCAGCCCTGGCTGGGGTGGTCTGAACTGTGAGCTG
GCGAGGGTCCAGTGCCCAGACCAGTGCAGTGGGCATGGCACGTACCTGCCTGACACGGGCCTCTGCAG
CTGCGATCCCAACTGGATGGGTCCCGACTGCTCTGTTGAAGTGTGCTCAGTAGACTGTGGCACTCACG
GCGTCTGCATCGGGGGAGCCTGCCGCTGTGAAGAGGGCTGGACAGGCGCAGCGTGTGACCAGCGCGTG
TGCCACCCCCGCTGCATTGAGCATGGGACCTGTAAAGATGGCAAATGTGAATGCCGAGAGGGCTGGAA
TGGTGAACACTGCACCATTGATGGCTGCCCTGACTTGTGCAACGGTAACGGGAGATGCACACTGGGTC
AGAACAGCTGGCAGTGTGTCTGCCAGACCGGCTGGAGAGGGCCCGGATGCAACGTTGCCATGGAAACT
TCCTGTGCTGATAACAAGGATAATGAGGGAGATGGCCTGGTGGATTGTTTGGACCCTGACTGCTGCCT
GCAGTCAGCCTGTCAGAACAGCCTGCTCTGCCGGGGGTCCCGGGACCCACTGGACATCATTCAGCAGG
GCCAGACGGATTGGCCCGCAGTGAAGTCCTTCTATGACCGTATCAAGCTCTTGGCAGGCAAGGATAGC
ACCCACATCATTCCTGGAGAGAACCCTTTCAACAGCAGCTTGGTTTCTCTCATCCGAGGCCAAGTAGT
AACTACAGATGGAACTCCCCTGGTCGGTGTGAACGTGTCTTTTGTCAAGTACCCAAAATACGGCTACA
CCATCACCCGCCAGGATGGCACGTTCGACCTGATCGCAAATGGAGGTGCTTCCTTGACTCTACACTTT
GAGCGAGCCCCGTTCATGAGCCAGGAGCGCACTGTGTGGCTGCCGTGGAACAGCTTTTACGCCATGGA
CACCCTGGTGATGAAGACCGAGGAGAACTCCATCCCCAGCTGTGACCTCAGTGGCTTTGTCCGGCCTG
ATCCAATCATCATCTCCTCCCCACTGTCCACCTTCTTTAGTGCTGCCCCTGGGCAGAATCCCATCGTG
CCTGAGACCCAGGTTCTTCATGAAGAAATCGAGCTCCCTGGTTCCAATGTGAAACTTCGCTATCTGAG
CTCTAGAACTGCAGGGTACAAGTCACTGCTGAAGATCACCATGACCCAGTCCACAGTGCCCCTGAACC
TCATTAGGGTTCACCTGATGGTGGCTGTCGAGGGGCATCTCTTCCAGAAGTCATTCCAGGCTTCTCCC
AACCTGGCCTCCACCTTCATCTGGGACAAGACAGATGCGTATGGCCAAAGGGTGTATGGACTCTCAGA
TGCTGTTGTGTCTGTCGGGTTTGAATATGAGACCTGTCCCAGTCTAATTCTCTGGGAGAAAAGGACAG
CCCTCCTTCAGGGATTCGAGCTGGACCCCTCCAACCTCGGTGGCTGGTCCCTAGACAAACACCACATC
CTCAATGTTAAAAGTGGAATCCTACACAAAGGCACTGGGGAAAACCAGTTCCTGACCCAGCAGCCTGC
CATCATCACCAGCATCATGGGCAATGGTCGCCGCCGGAGCATTTCCTGTCCCAGCTGCAACGGCCTTG
CTGAAGGCAACAAGCTGCTGGCCCCAGTGGCTCTGGCTGTTGGAATCGATGGGAGCCTCTATGTGGGT
GACTTCAATTACATCCGACGCATCTTTCCCTCTCGAAATGTGACCAGCATCTTGGAGTTACGAAATAA
AGAGTTTAAACATAGCAACAACCCAGCACACAAGTACTACTTGGCAGTGGACCCCGTGTCCGGCTCGC
TCTACGTGTCCGACACCAACAGCAGGAGAATCTACCGCGTCAAGTCTCTGAGTGGAACCAAAGACCTG
GCTGGGAATTCGGAAGTTGTGGCAGGGACGGGAGAGCAGTGTCTACCCTTTGATGAAGCCCGCTGCGG
GGATGGAGGGAAGGCCATAGATGCAACCCTGATGAGCCCGAGAGGTATTGCAGTAGACAAGAATGGGC
TCATGTACTTTGTCGATGCCACCATGATCCGGAAGGTTGACCAGAATGGAATCATCTCCACCCTGCTG
GGCTCCAATGACCTCACTGCCGTCCGGCCGCTGAGCTGTGATTCCAGCATGGATGTAGCCCAGGTTCG
TCTGGAGTGGCCAACAGACCTTGCTGTCAATCCCATGGATAACTCCTTGTATGTTCTAGAGAACAATG
TCATCCTTCGAATCACCGAGAACCACCAAGTCAGCATCATTGCGGGACGCCCCATGCACTGCCAAGTT
CCTGGCATTGACTACTCACTCAGCAAACTAGCCATTCACTCTGCCCTGGAGTCAGCCAGTGCCATTGC
CATTTCTCACACTGGGGTCCTCTACATCACTGAGACAGATGAGAAGAAGATTAACCGTCTACGCCAGG
TAACAACCAACGGGGAGATCTGCCTTTTAGCTGGGGCAGCCTCGGACTGCGACTGCAAAAACGATGTC
AATTGCAACTGCTATTCAGGAGATGATGCCTACGCGACTGATGCCATCTTGAATTCCCCATCATCCTT
AGCTGTAGCTCCAGATGGTACCATTTACATTGCAGACCTTGGAAATATTCGGATCAGGGCGGTCAGCA
AGAACAAGCCTGTTCTTAATGCCTTCAACCAGTATGAGGCTGCATCCCCCGGAGAGCAGGAGTTATAT
GTTTTCAACGCTGATGGCATCCACCAATACACTGTGAGCCTGGTGACAGGGGAGTACTTGTACAATTT
CACATATAGTACTGACAATGATGTCACTGAATTGATTGACAATAATGGGAATTCCCTGAAGATCCGTC
GGGACAGCAGTGGCATGCCCCGTCACCTGCTCATGCCTGACAACCAGATCATCACCCTCACCGTGGGC
ACCAATGGAGGCCTCAAAGTCGTGTCCACACAGAACCTGGAGCTTGGTCTCATGACCTATGATGGCAA
CACTGGGCTCCTGGCCACCAAGAGCGATGAAACAGGATGGACGACTTTCTATGACTATGACCACGAAG
GCCGCCTGACCAACGTGACGCGCCCCACGGGGGTGGTAACCAGTCTGCACCGGGAAATGGAGAAATCT
ATTACCATTGACATTGAGAACTCCAACCGTGATGATGACGTCACTGTCATCACCAACCTCTCTTCAGT
AGAGGCCTCCTACACAGTGGTACAAGATCAAGTTCGGAACAGCTACCAGCTCTGTAATAATGGTACCC
TGAGGGTGATGTATGCTAATGGGATGGGTATCAGCTTCCACAGCGAGCCCCATGTCCTAGCGGGCACC
ATCACCCCCACCATTGGACGCTGCAACATCTCCCTGCCTATGGAGAATGGCTTAAACTCCATTGAGTG
GCGCCTAAGAAAGGAACAGATTAAAGGCAAAGTCACCATCTTTGGCAGGAAGCTCCGGGTCCATGGAA
GAAATCTCTTGTCCATTGACTATGATCGAAATATTCGGACTGAAAAGATCTATGATGACCACCGGAAG
TTCACCCTGAGGATCATTTATGACCAGGTGGGCCGCCCCTTCCTCTGGCTGCCCAGCAGCGGGCTGGC
AGCTGTCAACGTGTCATACTTCTTCAATGGGCGCCTGGCTGGGCTTCAGCGTGGGGCCATGAGCGAGA
GGACAGACATCGACAAGCAAGGCCGCATCGTGTCCCGCATGTTCGCTGACGGGAAAGTGTGGAGCTAC
TCCTACCTTGACAAGTCCATGGTCCTCCTGCTTCAGAGCCAACGTCAGTATATATTTGAGTATGACTC
CTCTGACCGCCTCCTTGCCGTCACCATGCCCAGCGTGGCCCGGCACAGCATGTCCACACACACCTCCA
TCGGCTACATCCGTAATATTTACAACCCGCCTGAAAGCAATGCTTCGGTCATCTTTGACTACAGTGAT
GACGGCCGCATCCTGAAGACCTCCTTTTTGGGCACCGGACGCCAGGTGTTCTACAAGTATGGGAAACT
CTCCAAGTTATCAGAGATTGTCTACGACAGTACCGCCGTCACCTTCGGGTATGACGAGACCACTGGTG
TCTTGAAGATGGTCAACCTCCAAAGTGGGGGCTTCTCCTGCACCATCAGGTACCGGAAGATTGGCCCC
CTGGTGGACAAGCAGATCTACAGGTTCTCCGAGGAAGGCATGGTCAATGCCAGGTTTGACTACACCTA
TCATGACAACAGCTTCCGCATCGCAAGCATCAAGCCCGTCATAAGTGAGACTCCCCTCCCCGTTGACC
TCTACCGCTATGATGAGATTTCTGGCAAGGTGGAACACTTTGGTAAGTTTGGAGTCATCTATTATGAC
ATCAACCAGATCATCACCACTGCCGTGATGACCCTCAGCAAACACTTCGACACCCATGGGCGGATCAA
GGAGGTCCAGTATGAGATGTTCCGGTCCCTCATGTACTGGATGACGGTGCAATATGACAGCATGGGCA
GGGTGATCAAGAGGGAGCTAAAACTGGGGCCCTATGCCAATACCACGAAGTACACCTATGACTACGAT
GGGGACGGGCAGCTCCAGAGCGTGGCCGTCAATGACCGCCCGACCTGGCGCTACAGCTATGACCTTAA
TGGGAATCTCCACTTACTGAACCCAGGCAACAGTGTGCGCCTCATGCCCTTGCGCTATGACCTCCGGG
ATCGGATAACCAGACTCGGGGATGTGCAGTACAAAATTGACGACGATGGCTATCTGTGCCAGAGAGGG
TCTGACATCTTCGAATACAATTCCAAGGGCCTCCTAACAAGAGCCTACAACAAGGCCAGCGGGTGGAG
TGTCCAGTACCGCTATGATGGCGTAGGACGGCGGGCTTCCTACAAGACCAACCTGGGCCACCACCTGC
AGTACTTCTACTCTGACCTCCACAACCCGACGCGCATCACCCATGTCTACAATCACTCCAACTCGGAG
ATTACCTCACTGTACTACGACCTCCAGGGCCACCTCTTTGCCATGGAGAGCAGCAGTGGGGAGGAGTA
CTATGTTGCCTCTGATAACACAGGGACTCCTCTGGCTGTGTTCAGCATCAACGGCCTCATGATCAAAC
AGCTGCAGTACACGGCCTATGGGGAGATTTATTATGACTCCAACCCCGACTTCCAGATGGTCATTGGC
TTCCATGGGGGACTCTATGACCCCCTGACCAAGCTGGTCCACTTCACTCAGCGTGATTATGATGTGCT
GGCAGGACGATGGACCTCCCCAGACTATACCATGTGGAAAAACGTGGGCAAGGAGCCGGCCCCCTTTA
ACCTGTATATGTTCAAGAGCAACAATCCTCTCAGCAGTGAGCTAGATTTGAAGAACTACGTGACAGAT
GTGAAAAGCTGGCTTGTGATGTTTGGATTTCAGCTTAGCAACATCATTCCTGGCTTCCCGAGAGCCAA
AATGTATTTCGTGCCTCCTCCCTATGAATTGTCAGAGAGTCAAGCAAGTGAGAATGGACAGCTCATTA
CAGGTGTCCAACAGACAACAGAGAGACATAACCAGGCCTTCATGGCTCTGGAAGGACAGGTCATTACT
AAAAAGCTCCACGCCAGCATCCGAGAGAAAGCAGGTCACTGGTTTGCCACCACCACGCCCATCATTGG
CAAAGGCATCATGTTTGCCATCAAAGAAGGGCGGGTGACCACGGGCGTGTCCAGCATCGCCAGCGAAG
ATAGCCGCAAGGTGGCATCTGTGCTGAACAACGCCTACTACCTGGACAAGATGCACTACAGCATCGAG
GGCAAGGACACCCACTACTTTGTGAAGATTGGCTCAGCCGATGGCGACCTGGTCACACTAGGCACCAC
CATCGGCCGCAAGGTGCTAGAGAGCGGGGTGAACGTGACCGTGTCCCAGCCCACGCTGCTGGTCAACG
GCAGGACTCGAAGGTTCACGAACATTGAGTTCCAGTACTCCACGCTGCTGCTCAGCATCCGCTATGGC
CTCACCCCCGACACCCTGGACGAAGAGAAGGCCCGCGTCCTGGACCAGGCGAGACAGAGGGCCCTGGG
CACGGCCTGGGCCAAGGAGCAGCAGAAAGCCAGGGACGGGAGAGAGGGGAGCCGCCTGTGGACTGAGG
GCGAGAAGCAGCAGCTTCTGAGCACCGGGCGCGTGCAAGGGTACGAGGGATATTACGTGCTTCCCGTG
GAGCAATACCCAGAGCTTGCAGACAGTAGCAGCAACATCCAGTTTTTAAGACAGAATGAGATGGGAAA
GAGGTAACAAAATAATCTGCTGCCATTCCTTGTCTGAATGGCTCAGCAGGAGTAACTGTTATCTCCTC
TCCTAAGGAGATGAAGACCTAACAGGGGCACTGCGGCTGGGCTGCTTTAGGAGACCAAGTGGCAAGAA
AGCTCACATTTTTTGAGTTCAAATGCTACTGTCCAAGCGAGAAGTCCCTCATCCTGAAGTAGACTAAA
GCCCGGCTGAAAATTCCGAGGAAAACAAAACAAACGAATGAATGAACAGACACACACAATGTTCCAAC
TTCCCCTAAAATATGACCCACTTGTTCTGGGTCTACGCAGAAAAGAGACGCAAAGTGTCCAAAAGGAA
CAAAAGAACAAAAACGAATAAGCAAAGAAGAAAACAAACAAAAACAAAACAAAACAAACACACGGACC
GATAAACAAAGAAGCGAAGATAAGAAAGAAGGCCTCATATCCAATTACCTCACTCATTCACATGTGAG
CGACACGCAGACATCCGCGAGGGCCAGCGTCACCAGACCAGCTGCGGGACAAACCACTCAGACTGCTT
GTAGGACAAATACTTCTGACATTTTCGTTTAAGCAAATACAGGTGCATTTAAAACACGACTTTGGGGG
TGATTTGTGTGTAGCGCCTGGGGAGGGGGGATAAAAGAGGAGGAGTGAGCACTGGAAATACTTTTTAA
AGAAAAAAAAACATGAGGGAATAAAAGAAATTCCTATCAAAAATCAAAGTGAAATAATACCATCCAGC
ACTTAACTCTCAGGTCCCAACTAAGTCTGGCCTGAGCTAATTTATTTGAGCGCAGAGTGTAAAATTTA
ATTCAAAATGGTGGCTATAATCACTACAGATAAATTTCATACTCTTTTGTCTTTGGAGATTCCATTGT
GGACAGTAATACGCAGTTACAGGGTGTAGTCTGTTTAGATTCCGTAGTTCGTGGGTATCAGTTTCGGT
AGAGGTGCAGCATCGTGACACTTTTGCTAACAGGTACCACTTCTGATCACCCTGTACATACATGAGCC
GAAAGGCACAATCACTGTTTCAGATTTAAAATTATTAGTGTGTTTGTTTGGTCCAGAAACTGAGACAA
TCACATGACAGTCACCACGAGGAGAGAAAATTTAAAAAATAAAAATAAAAACAAAAAAAATTTTAAAA
ATTAAAAAAACAAAAATAAAGTCTAATAAGAACTTTGGTACAGGAACTTTTTTGTAATATACATGTAT
GAATTGTTCATCGAGTTTTTATATTAATTTTAATTTGCTGCTAAGCAAAGACTAGGGACAGGCAAAGA
TAATTTATGGCAAAGTGTTTAAATTGTTTATACATAAATAAAGTCTCTAAAACTCCTGTG

| | | | |
|---|---|---|---|
| NOV7b, CG50426-21 | SEQ ID NO: 116 | 2765 aa | MW at 306806.1kD |
| Protein Sequence | | | |

MDVKDRRHRSLTRGRCGKECRYTSSSLDSEDCRVPTQKSYSSSETLKAYDHDSRMHYGNRVTDLIHRE
SDEFPRQGTNFTLAELGICEPSPHRSGYCSDMGILHQGYSLSTGSDADSDTEGGMSPEHAIRLWGRGI
KSRRSSGLSSRENSALTLTDSDNENKSDDENGRPIPPTSSPSLLPSAQLPSSHNPPPVSCQMPLLDSN
TSHQIMDTNPDEEFSPNSYLLRACSGPQQASSSGPPNHHSQSTLRPPLPPPHNHTLSHHHSSANSLNR
NSLTNRRSQIHAPAPAPNDLATTPESVQLQDSWVLNSNVPLETRHFLFKTSSGSTPLFSSSSPGYPLT
SGIVYTPPPRLLPRNTFSRKAFKLKKPSKYCSWKCAALSAIAAALLLAILLAYFIAMHLLGLNWQLQP
ADGHTFNNGIRTGLPGNDDVATMPSGGKVPWSLKNSSIDSGEAEVGRRVTQEVPPGVFWRSQIHISQP
QFLKFNISLGKDALFGVYIRRGLPPSHAQYDFMERLDGKEKWSVVESPRERRSIQTLVQNEAVFVQYL
DVGLWHLAFYNDGKDKEMVSFNTVVLDSVQDCPRNCHGNGECVSGVCHCFPGFLGADCAKAACPVLCS
GNGQYSKGTCQCYSGWKGAECDVPMNQCIDPSCGGHGSCIDGNCVCSAGYKGEHCEEVDCLDPTCSSH
GVCVNGECLCSPGWGGLNCELARVQCPDQCSGHGTYLPDTGLCSCDPNWMGPDCSVEVCSVDCGTHGV
CIGGACRCEEGWTGAACDQRVCHPRCIEHGTCKDGKCECREGWNGEHCTIDGCPDLCNGNGRCTLGQN
SWQCVCQTGWRGPGCNVAMETSCADNKDNEGDGLVDCLDPDCCLQSACQNSLLCRGSRDPLDIIQQGQ
TDWPAVKSFYDRIKLLAGKDSTHIIPGENPFNSSLVSLIRGQVVTTDGTPLVGVNVSFVKYPKYGYTI
TRQDGTFDLIANGGASLTLHFERAPFMSQERTVWLPWNSFYAMDTLVMKTEENSIPSCDLSGFVRPDP
IIISSPLSTFFSAAPGQNPIVPETQVLHEEIELPGSNVKLRYLSSRTAGYKSLLKITMTQSTVPLNLI
RVHLMVAVEGHLFQKSFQASPNLASTFIWDKTDAYGQRVYGLSDAVVSVGFEYETCPSLILWEKRTAL
LQGFELDPSNLGGWSLDKHHILNVKSGILHKGTGENQFLTQQPAIITSIMGNGRRRSISCPSCNGLAE
GNKLLAPVALAVGIDGSLYVGDFNYIRRIFPSRNVTSILELRNKEFKHSNNPAHKYYLAVDPVSGSLY
VSDTNSRRIYRVKSLSGTKDLAGNSEVVAGTGEQCLPFDEARCGDGGKAIDATLMSPRGIAVDKNGLM
YFVDATMIRKVDQNGIISTLLGSNDLTAVRPLSCDSSMDVAQVRLEWPTDLAVNPMDNSLYVLENNVI
LRITENHQVSIIAGRPMHCQVPGIDYSLSKLAIHSALESASAIAISHTGVLYITETDEKKINRLRQVT
TNGEICLLAGAASDCDCKNDVNCNCYSGDDAYATDAILNSPSSLAVAPDGTIYIADLGNIRIRAVSKN
KPVLNAFNQYEAASPGEQELYVFNADGIHQYTVSLVTGEYLYNFTYSTDNDVTELIDNNGNSLKIRRD
SSGMPRHLLMPDNQIITLTVGTNGGLKVVSTQNLELGLMTYDGNTGLLATKSDETGWTTFYDYDHEGR
LTNVTRPTGVVTSLHREMEKSITIDIENSNRDDDVTVITNLSSVEASYTVVQDQVRNSYQLCNNGTLR
VMYANGMGISFHSEPHVLAGTITPTIGRCNISLPMENGLNSIEWRLRKEQIKGKVTIFGRKLRVHGRN
LLSIDYDRNIRTEKIYDDHRKFTLRIIYDQVGRPFLWLPSSGLAAVNVSYFFNGRLAGLQRGAMSERT
DIDKQGRIVSRMFADGKVWSYSYLDKSMVLLLQSQRQYIFEYDSSDRLLAVTMPSVARHSMSTHTSIG
YIRNIYNPPESNASVIFDYSDDGRILKTSFLGTGRQVFYKYGKLSKLSEIVYDSTAVTFGYDETTGVL
KMVNLQSGGFSCTIRYRKIGPLVDKQIYRFSEEGMVNARFDYTYHDNSFRIASIKPVISETPLPVDLY
RYDEISGKVEHFGKFGVIYYDINQIITTAVMTLSKHFDTHGRIKEVQYEMFRSLMYWMTVQYDSMGRV
IKRELKLGPYANTTKYTYDYDGDGQLQSVAVNDRPTWRYSYDLNGNLHLLNPGNSVRLMPLRYDLRDR
ITRLGDVQYKIDDDGYLCQRGSDIFEYNSKGLLTRAYNKASGWSVQYRYDGVGRRASYKTNLGHHLQY
FYSDLHNPTRITHVYNHSNSEITSLYYDLQGHLFAMESSSGEEYYVASDNTGTPLAVFSINGLMIKQL
QYTAYGEIYYDSNPDFQMVIGFHGGLYDPLTKLVHFTQRDYDVLAGRWTSPDYTMWKNVGKEPAPFNL
YMFKSNNPLSSELDLKNYVTDVKSWLVMFGFQLSNIIPGFPRAKMYFVPPPYELSESQASENGQLITG
VQQTTERHNQAFMALEGQVITKKLHASIREKAGHWFATTTPIIGKGIMFAIKEGRVTTGVSSIASEDS
RKVASVLNNAYYLDKMHYSIEGKDTHYFVKIGSADGDLVTLGTTIGRKVLESGVNVTVSQPTLLVNGR
TRRFTNIEFQYSTLLLSIRYGLTPDTLDEEKARVLDQARQRALGTAWAKEQQKARDGREGSRLWTEGE
IKQQLLSTGRVQGYEGYYVLPVEQYPELADSSSNIQFLRQNEMGKR

| | | |
|---|---|---|
| NOV7c, CG50426-15 | SEQ ID NO: 117 | 2478 bp |
| DNA Sequence | ORF Start: at 1 | ORF Stop: end of sequence |

AAAAACAGCAGCATAGACAGTGGTGAAGCAGAAGTTGGTCGGCGGGTAACACAAGAAGTCCCACCAGG
GGTGTTTTGGAGGTCACAAATTCACATCAGTCAGCCCCAGTTCTTAAAGTTCAACATCTCCCTCGGGA
AGGACGCTCTCTTTGGTGTTTACATAAGAAGAGGACTTCCACCATCTCATGCCCAGTATGACTTCATG
GAACGTCTGGACGGGAAGGAGAAGTGGAGTGTGGTTGAGTCTCCCAGGGAACGCCGGAGCATACAGAC
CTTGGTTCAGAATGAAGCCGTGTTTGTGCAGTACCTGGATGTGGGCCTGTGGCATCTGGCCTTCTACA
ATGATGGAAAAGACAAAGAGATGGTTTCCTTCAATACTGTTGTCCTAGATTCAGTGCAGGACTGTCCA
CGTAACTGCCATGGGAATGGTGAATGTGTGTCCGGGGTGTGTCACTGTTTCCCAGGATTTCTAGGAGC
AGACTGTGCTAAAGCTGCCTGCCCTGTCCTGTGCAGTGGGAATGGACAATATTCTAAAGGGACGTGCC
AGTGCTACAGCGGCTGGAAAGGTGCAGAGTGCGACGTGCCCATGAATCAGTGCATCGATCCTTCCTGC
GGGGGCCACGGCTCCTGCATTGATGGGAACTGTGTCTGCTCTGCTGGCTACAAAGGCGAGCACTGTGA
GGAAGTTGATTGCTTGGATCCCACCTGCTCCAGCCACGGAGTCTGTGTGAATGGAGAATGCCTGTGCA
GCCCTGGCTGGGGTGGTCTGAACTGTGAGCTGGCGAGGGTCCAGTGCCCAGACCAGTGCAGTGGGCAT
GGCACGTACCTGCCTGACACGGGCCTCTGCAGCTGCGATCCCAACTGGATGGGTCCCGACTGCTCTGT
TGAAGTGTGCTCAGTAGACTGTGGCACTCACGGCGTCTGCATCGGGGGAGCCTGCCGCTGTGAAGAGG
GCTGGACAGGCGCAGCGTGTGACCAGCGCGTGTGCCACCCCCGCTGCATTGAGCACGGGACCTGTAAA
GATGGCAAATGTGAATGCCGAGAGGGCTGGAATGGTGAACACTGCACCATTGGTAGGCAAACGGCAGG
CACCGAAACAGATGGCTGCCCTGACTTGTGCAACGGTAACGGGAGATGCACACTGGGTCAGAACAGCT
GGCAGTGTGTCTGCCAGACCGGCTGGAGAGGGCCCGGATGCAACGTTGCCATGGAAACTTCCTGTGCT
GATAACAAGGATAATGAGGGAGATGGCCTGGTGGATTGTTTGGACCCTGACTGCTGCCTGCAGTCAGC
CTGTCAGAACAGCCTGCTCTGCCGGGGGTCCCGGGACCCACTGGACATCATTCAGCAGGGCCAGACGG
ATTGGCCCGCAGTGAAGTCCTTCTATGACCGTATCAAGCTCTTGGCAGGCAAGGATAGCACCCACATC
ATTCCTGGAGAGAACCCTTTCAACAGCAGCTTGGTTTCTCTCATCCGAGGCCAAGTAGTAACTACAGA
TGGAACTCCCCTGGTCGGTGTGAACGTGTCTTTTGTCAAGTACCCAAAATACGGCTACACCATCACCC
GCCAGGATGGCACGTTCGACCTGATCGCAAATGGAGGTGCTTCCTTGACTCTACACTTTGAGCGAGCC
CCGTTCATGAGCCAGGAGCGCACTGTGTGGCTGCCGTGGAACAGCTTTTACGCCATGGACACCCTGGT
GATGAAGACCGAGGAGAACTCCATCCCCAGCTGTGACCTCAGTGGCTTTGTCCGGCCTGATCCAATCA
TCATCTCCTCCCCACTGTCCACCTTCTTTAGTGCTGCCCCTGGGCAGAATCCCATCGTGCCTGAGACC
CAGGTTCTTCATGAAGAAATCGAGCTCCCTGGTTCCAATGTGAAACTTCGCTATCTGAGCTCTAGAAC
TGCAGGGTACAAGTCACTGCTGAAGATCACCATGACCCAGTCCACAGTGCCCCTGAACCTCATTAGGG
TTCACCTGATGGTGGCTGTCGAGGGGCATCTCTTCCAGAAGTCATTCCAGGCTTCTCCCAACCTGGCC
TCCACCTTCATCTGGGACAAGACAGATGCGTATGGCCAAAGGGTGTATGGACTCTCAGATGCTGTTGT
GTCTGTCGGGTTTGAATATGAGACCTGTCCCAGTCTAATTCTCTGGGAGAAAAGGACAGCCCTCCTTC
AGGGATTCGAGCTGGACCCCTCCAACCTCGGTGGCTGGTCCCTAGACAAACACCACATCCTCAATGTT
AAAAGTGGAATCCTACACAAAGGCACTGGGGAAAACCAGTTCCTGACCCAGCAGCCTGCCATCATCAC
CAGCATCATGGGCAATGGTCGCCGCCGGAGCATTTCCTGTCCCAGCTGCAACGGCCTTGCTGAAGGCA
ACAAGCTGCTGGCCCCAGTGGCTCTGGCTGTTGGAATCGATGGGAGCCTCTATGTGGGTGACTTCAAT
TACATCCGACGCATCTTTCCCTCTCGAAAT

| | | | |
|---|---|---|---|
| NOV7c, CG50426-15 | SEQ ID NO: 118 | 826 aa | MW at 89833.6kD |
| Protein Sequence | | | |

KNSSIDSGEAEVGRRVTQEVPPGVFWRSQIHISQPQFLKFNISLGKDALFGVYIRRGLPPSHAQYDFM
ERLDGKEKWSVVESPRERRSIQTLVQNEAVFVQYLDVGLWHLAFYNDGKDKEMVSFNTVVLDSVQDCP
RNCHGNGECVSGVCHCFPGFLGADCAKAACPVLCSGNGQYSKGTCQCYSGWKGAECDVPMNQCIDPSC
GGHGSCIDGNCVCSAGYKGEHCEEVDCLDPTCSSHGVCVNGECLCSPGWGGLNCELARVQCPDQCSGH
GTYLPDTGLCSCDPNWMGPDCSVEVCSVDCGTHGVCIGGACRCEEGWTGAACDQRVCHPRCIEHGTCK
DGKCECREGWNGEHCTIGRQTAGTETDGCPDLCNGNGRCTLGQNSWQCVCQTGWRGPGCNVAMETSCA
DNKDNEGDGLVDCLDPDCCLQSACQNSLLCRGSRDPLDIIQQGQTDWPAVKSFYDRIKLLAGKDSTHI
IPGENPFNSSLVSLIRGQVVTTDGTPLVGVNVSFVKYPKYGYTITRQDGTFDLIANGGASLTLHFERA
PFMSQERTVWLPWNSFYAMDTLVMKTEENSIPSCDLSGFVRPDPIIISSPLSTFFSAAPGQNPIVPET
QVLHEEIELPGSNVKLRYLSSRTAGYKSLLKITMTQSTVPLNLIRVHLMVAVEGHLFQKSFQASPNLA
STFIWDKTDAYGQRVYGLSDAVVSVGFEYETCPSLILWEKRTALLQGFELDPSNLGGWSLDKHHILNV
KSGILHKGTGENQFLTQQPAIITSIMGNGRRRSISCPSCNGLAEGNKLLAPVALAVGIDGSLYVGDFN
YIRRIFPSRN

| | | |
|---|---|---|
| NOV7d, 306276924 | SEQ ID NO: 119 | 2497 bp |
| DNA Sequence | ORF Start: at 2 | ORF Stop: end of sequence |

CACCAAGCTTAAAAACAGCAGCATAGACAGTGGTGAAGCAGAAGTTGGTCGGCGGGTAACACAAGAAG
TCCCACCAGGGGTGTTTTGGAGGTCACAAATTCACATCAGTCAGCCCCAGTTCTTAAAGTTCAACATC
TCCCTCGGGAAGGACGCTCTCTTTGGTGTTTACATAAGAAGAGGACTTCCACCATCTCATGCCCAGTA
TGACTTCATGGAACGTCTGGACGGGAAGGAGAAGTGGAGTGTGGTTGAGTCTCCCAGGGAACGCCGGA
GCATACAGACCTTGGTTCAGAATGAAGCCGTGTTTGTGCAGTACCTGGATGTGGGCCTGTGGCATCTG
GCCTTCTACAATGATGGAAAAGACAAAGAGATGGTTTCCTTCAATACTGTTGTCCTAGATTCAGTGCA
GGACTGTCCACGTAACTGCCATGGGAATGGTGAATGTGTGTCCGGGGTGTGTCACTGTTTCCCAGGAT
TTCTAGGAGCAGACTGTGCTAAAGCTGCCTGCCCTGTCCTGTGCAGTGGGAATGGACAATATTCTAAA
GGGACGTGCCAGTGCTACAGCGGCTGGAAAGGTGCAGAGTGCGACGTGCCCATGAATCAGTGCATCGA
TCCTTCCTGCGGGGGCCACGGCTCCTGCATTGATGGGAACTGTGTCTGCTCTGCTGGCTACAAAGGCG
AGCACTGTGAGGAAGTTGATTGCTTGGATCCCACCTGCTCCAGCCACGGAGTCTGTGTGAATGGAGAA
TGCCTGTGCAGCCCTGGCTGGGGTGGTCTGAACTGTGAGCTGGCGAGGGTCCAGTGCCCAGACCAGTG
CAGTGGGCATGGCACGTACCTGCCTGACACGGGCCTCTGCAGCTGCGATCCCAACTGGATGGGTCCCG
ACTGCTCTGTTGAAGTGTGCTCAGTAGACTGTGGCACTCACGGCGTCTGCATCGGGGGAGCCTGCCGC
TGTGAAGAGGGCTGGACAGGCGCAGCGTGTGACCAGCGCGTGTGCCACCCCCGCTGCATTGAGCACGG
GACCTGTAAAGATGGCAAATGTGAATGCCGAGAGGGCTGGAATGGTGAACACTGCACCATTGGTAGGC
AAACGGCAGGCACCGAAACAGATGGCTGCCCTGACTTGTGCAACGGTAACGGGAGATGCACACTGGGT
CAGAACAGCTGGCAGTGTGTCTGCCAGACCGGCTGGAGAGGGCCCGGATGCAACGTTGCCATGGAAAC
TTCCTGTGCTGATAACAAGGATAATGAGGGAGATGGCCTGGTGGATTGTTTGGACCCTGACTGCTGCC
TGCAGTCAGCCTGTCAGAACAGCCTGCTCTGCCGGGGGTCCCGGGACCCACTGGACATCATTCAGCAG
GGCCAGACGGATTGGCCCGCAGTGAAGTCCTTCTATGACCGTATCAAGCTCTTGGCAGGCAAGGATAG
CACCCACATCATTCCTGGAGAGAACCCTTTCAACAGCAGCTTGGTTTCTCTCATCCGAGGCCAAGTAG
TAACTACAGATGGAACTCCCCTGGTCGGTGTGAACGTGTCTTTTGTCAAGTACCCAAAATACGGCTAC
ACCATCACCCGCCAGGATGGCACGTTCGACCTGATCGCAAATGGAGGTGCTTCCTTGACTCTACACTT
TGAGCGAGCCCCGTTCATGAGCCAGGAGCGCACTGTGTGGCTGCCGTGGAACAGCTTTTACGCCATGG
ACACCCTGGTGATGAAGACCGAGGAGAACTCCATCCCCAGCTGTGACCTCAGTGGCTTTGTCCGGCCT
GATCCAATCATCATCTCCTCCCCACTGTCCACCTTCTTTAGTGCTGCCCCTGGGCAGAATCCCATCGT
GCCTGAGACCCAGGTTCTTCATGAAGAAATCGAGCTCCCTGGTTCCAATGTGAAACTTCGCTATCTGA
GCTCTAGAACTGCAGGGTACAAGTCACTGCTGAAGATCACCATGACCCAGTCCACAGTGCCCCTGAAC
CTCATTAGGGTTCACCTGATGGTGGCTGTCGAGGGGCATCTCTTCCAGAAGTCATTCCAGGCTTCTCC
CAACCTGGCCTACACCTTCATCTGGGACAAGACAGATGCGTATGGCCAAAGGGTGTATGGACTCTCAG
ATGCTGTTGTGTCTGTCGGGTTTGAATATGAGACCTGTCCCAGTCTAATTCTCTGGGAGAAAAGGACA
GCCCTCCTTCAGGGATTCGAGCTGGACCCCTCCAACCTCGGTGGCTGGTCCCTAGACAAACACCACAT
CCTCAATGTTAAAAGTGGAATCCTACACAAAGGCACTGGGGAAAACCAGTTCCTGACCCAGCAGCCTG
CCATCATCACCAGCATCATGGGCAATGGTCGCCGCCGGAGCATTTCCTGTCCCAGCTGCAACGGCCTT
GCTGAAGGCAACAAGCTGCTGGCCCCAGTGGCTCTGGCTGTTGGAATCGATGGGAGCCTCTATGTGGG
TGACTTCAATTACATCCGACGCATCTTTCCCTCTCGAAATCTCGAGGGC

| | | | |
|---|---|---|---|
| NOV7d, 306276924 | SEQ ID NO: 120 | 832 aa | MW at 90551.4kD |
| Protein Sequence | | | |

TKLKNSSIDSGEAEVGRRVTQEVPPGVFWRSQIHISQPQFLKFNISLGKDALFGVYIRRGLPPSHAQY
DFMERLDGKEKWSVVESPRERRSIQTLVQNEAVFVQYLDVGLWHLAFYNDGKDKEMVSFNTVVLDSVQ
DCPRNCHGNGECVSGVCHCFPGFLGADCAKAACPVLCSGNGQYSKGTCQCYSGWKGAECDVPMNQCID
PSCGGHGSCIDGNCVCSAGYKGEHCEEVDCLDPTCSSHGVCVNGECLCSPGWGGLNCELARVQCPDQC
SGHGTYLPDTGLCSCDPNWMGPDCSVEVCSVDCGTHGVCIGGACRCEEGWTGAACDQRVCHPRCIEHG
TCKDGKCECREGWNGEHCTIGRQTAGTETDGCPDLCNGNGRCTLGQNSWQCVCQTGWRGPGCNVAMET
SCADNKDNEGDGLVDCLDPDCCLQSACQNSLLCRGSRDPLDIIQQGQTDWPAVKSFYDRIKLLAGKDS
THIIPGENPFNSSLVSLIRGQVVTTDGTPLVGVNVSFVKYPKYGYTITRQDGTFDLIANGGASLTLHF
ERAPFMSQERTVWLPWNSFYAMDTLVMKTEENSIPSCDLSGFVRPDPIIISSPLSTFFSAAPGQNPIV
PETQVLHEEIELPGSNVKLRYLSSRTAGYKSLLKITMTQSTVPLNLIRVHLMVAVEGHLFQKSFQASP
NLAYTFIWDKTDAYGQRVYGLSDAVVSVGFEYETCPSLILWEKRTALLQGFELDPSNLGGWSLDKHHI
LNVKSGILHKGTGENQFLTQQPAIITSIMGNGRRRSISCPSCNGLAEGNKLLAPVALAVGIDGSLYVG
DFNYIRRIFPSRNLEG

| | | |
|---|---|---|
| NOV7e, 306276936 | SEQ ID NO: 121 | 2470 bp |
| DNA Sequence | ORF Start: at 2 | ORF Stop: end of sequence |

CACCAAGCTTAAAAACAGCAGCATAGACAGTGGTGAAGCAGAAGTTGGTCGGCGGGTAACACAAGAAG
TCCCACCAGGGGTGTTTTGGAGGTCACAAATTCACATCAGTCAGCCCCAGTTCTTAAAGTTCAACATC
TCCCTCGGGAAGGACGCTCTCTTTGGTGTTTACATAAGAAGAGGACTTCCACCATCTCATGCCCAGTA
TGACTTCATGGAACGTCTGGACGGGAAGGAGAAGTGGAGTGTGGTTGAGTCTCCCAGGGAACGCCGGA
GCATACAGACCTTGGTTCAGAATGAAGCCGTGTTTGTGCAGTACCTGGATGTGGGCCTGTGGCATCTG
GCCTTCTACAATGATGGAAAAGACAAAGAGATGGTTTCCTTCAATACTGTTGTCCTAGATTCAGTGCA
GGACTGTCCACGTAACTGCCATGGGAATGGTGAATGTGTGTCCGGGGTGTGTCACTGTTTCCCAGGAT
TTCTAGGAGCAGACTGTGCTAAAGCTGCCTGCCCTGTCCTGTGCAGTGGGAATGGACAATATTCTAAA
GGGACGTGCCAGTGCTACAGCGGCTGGAAAGGTGCAGAGTGCGACGTGCCCATGAATCAGTGCATCGA
TCCTTCCTGCGGGGGCCACGGCTCCTGCATTGATGGGAACTGTGTCTGCTCTGCTGGCTACAAAGGCG
AGCACTGTGAGGAAGTTGATTGCTTGGATCCCACCTGCTCCAGCCACGGAGTCTCTGTGAATGGAGAA
TGCCTGTGCAGCCCTGGCTGGGGTGGTCTGAACTGTGAGCTGGCGAGGGTCCAGTGCCCAGACCAGTG
CAGTGGGCATGGCACGTACCTGCCTGACACGGGCCTCTGCAGCTGCGATCCCAACTGGATGGGTCCCG
ACTGCTCTGTTGAAGTGTGCTCAGTAGACTGTGGCACTCACGGCGTCTGCATCGGGGGAGCCTGCCGC
TGTGAAGAGGGCTGGACAGGCGCAGCGTGTGACCAGCGCGTGTGCCACCCCCGCTGCATTGAGCACGG
GACCTGTAAAGATGGCAAATGTGAATGCCGAGAGGGCTGGAATGGTGAACACTGCACCATTGATGGCT
GCCCTGACTTGTGCAACGGTAACGGGAGATGCACACTGGGTCAGAACAGCTGGCAGTGTGTCTGCCAG
ACCGGCTGGAGAGGGCCCGGATGCAACGTTGCCATGGAAACTTCCTGTGCTGATAACAAGGATAATGA
GGGAGATGGCCTGGTGGATTGTTTGGACCCTGACTGCTGCCTGCAGTCAGCCTGTCAGAACAGCCTGC
TCTGCCGGGGGTCCCGGGACCCACTGGACATCATTCAGCAGGGCCAGACGGATTGGCCCGCAGTGAAG
TCCTTCTATGACCGTATCAAGCTCTTGGCAGGCAAGGATAGCACCCACATCATTCCTGGAGAGAACCC
TTTCAACAGCAGCTTGGTTTCTCTCATCCGAGGCCAAGTAGTAACTACAGATGGAACTCCCCTGGTCG
GTGTGAACGTGTCTTTTGTCAAGTACCCAAAATACGGCTACACCATCACCCGCCAGGATGGCACGTTC
GACCTGATCGCAAATGGAGGTGCTTCCTTGACTCTACACTTTGAGCGAGCCCCGTTCATGAGCCAGGA
GCGCACTGTGTGGCTGCCGTGGAACAGCTTTTACGCCATGGACACCCTGGTGATGAAGACCGAGGAGA
ACTCCATCCCCAGCTGTGACCTCAGTGGCTTTGTCCGGCCTGATCCAATCATCATCTCCTCCCCACTG
TCCACCTTCTTTAGTGCTGCCCCTGGGCAGAATCCCATCGTGCCTGAGACCCAGGTTCTTCATGAAGA
AATCGAGCTCCCTGGTTCCAATGTGAAACTTCGCTATCTGAGCTCTAGAACTGCAGGGTACAAGTCAC
TGCTGAAGATCACCATGACCCAGTCCACAGTGCCCCTGAACCTCATTAGGGTTCACCTGATGGTGGCT
GTCGAGGGGCATCTCTTCCAGAAGTCATTCCAGGCTTCTCCCAACCTGGCCTACACCTTCATCTGGGA
CAAGACAGATGCGTATGGCCAAAGGGTGTATGGACTCTCAGATGCTGTTGTGTCTGTCGGGTTTGAAT
ATGAGACCTGTCCCAGTCTAATTCTCTGGGAGAAAAGGACAGCCCTCCTTCAGGGATTCGAGCTGGAC
CCCTCCAACCTCGGTGGCTGGTCCCTAGACAAACACCACATCCTCAATGTTAAAAGTGGAATCCTACA
CAAAGGCACTGGGGAAAACCAGTTCCTGACCCAGCAGCCTGCCATCATCACCAGCATCATGGGCAATG
GTCGCCGCCGGAGCATTTCCTGTCCCAGCTGCAACGGCCTTGCTGAAGGCAACAAGCTGCTGGCCCCA
GTGGCTCTGGCTGTTGGAATCGATGGGAGCCTCTATGTGGGTGACTTCAATTACATCCGACGCATCTT
TCCCTCTCGAAATCTCGAGGGC

| | | | |
|---|---|---|---|
| NOV7e, 306276936 | SEQ ID NO: 122 | 823 aa | MW at 89649.5kD |
| Protein Sequence | | | |

TKLKNSSIDSGEAEVGRRVTQEVPPGVFWRSQIHISQPQFLKFNISLGKDALFGVYIRRGLPPSHAQY
DFMERLDGKEKWSVVESPRERRSIQTLVQNEAVFVQYLDVGLWHLAFYNDGKDKEMVSFNTVVLDSVQ
DCPRNCHGNGECVSGVCHCFPGFLGADCAKAACPVLCSGNGQYSKGTCQCYSGWKGAECDVPMNQCID
PSCGGHGSCIDGNCVCSAGYKGEHCEEVDCLDPTCSSHGVCVNGECLCSPGWGGLNCELARVQCPDQC
SGHGTYLPDTGLCSCDPNWMGPDCSVEVCSVDCGTHGVCIGGACRCEEGWTGAACDQRVCHPRCIEHG
TCKDGKCECREGWNGEHCTIDGCPDLCNGNGRCTLGQNSWQCVCQTGWRGPGCNVAMETSCADNKDNE
GDGLVDCLDPDCCLQSACQNSLLCRGSRDPLDIIQQGQTDWPAVKSFYDRIKLEAGKDSTHIIPGENP
FNSSLVSLIRGQVVTTDGTPLVGVNVSFVKYPKYGYTITRQDGTFDLIANGGASLTLHFERAPFMSQE
RTVWLPWNSFYAMDTLVMXTEENSIPSCDLSGFVRPDPIIISSPLSTFFSAAPGQNPIVPETQVLHEE
IELPGSNVKLRYLSSRTAGYKSLLKITNTQSTVPLNLIRVHLMVAVEGHLFQKSFQASPNLAYTFIWD
KTDAYGQRVYGLSDAVVSVGFEYETCPSLILWEKRTALLQGFELDPSNLGGWSLDKHHILNVKSGILH
KGTGENQFLTQQPAIITSIMGNGRRRSISCPSCNGLAEGNKLLAPVALAVGIDGSLYVGDFNYIRRIF
PSRNLEG

| | | |
|---|---|---|
| NOV7f, 308530526 | SEQ ID NO: 123 | 2470 bp |
| DNA Sequence | ORF Start: at 2 | ORF Stop: end of sequence |

CACCAAGCTTAAAAACAGCAGCATAGACAGTGGTGAAGCAGAAGTTGGTCGGCGGGTAACACAAGAAG
TCCCACCAGGGGTGTTTTGGAGGTCACAAATTCACATCAGTCAGCCCCAGTTCTTAAAGTTCAACATC
TCCCTCGGGAAGGACGCTCTCTTTGGTGTTTACATAAGAAGAGGACTTCCACCATCTCATGCCCAGTA
TGACTTCATGGAACGTCTGGACGGGAAGGAGAAGTGGAGTGTGGTTGAGTCTCCCAGGGAACGCCGGA
GCATACAGACCTTGGTTCAGAATGAAGCCGTGTTTGTGCAGTACCTGGATGTGGGCCTGTGGCATCTG
GCCTTCTACAATGATGGAAAAGACAAAGAGATGGTTTCCTTCAATACTGTTGTCCTAGATTCAGTGCA
GGACTGTCCACGTAACTGCCATGGGAATGGTGAATGTGTGTCCGGGGTGTGTCACTGTTTCCCAGGAT
TTCTAGGAGCAGACTGTGCTAAAGCTGCCTGCCCTGTCCTGTGCAGTGGGAATGGACAATATTCTAAA
GGGACGTGCCAGTGCTACAGCGGCTGGAAAGGTGCAGAGTGCGACGTGCCCATGAATCAGTGCATCGA
TCCTTCCTGCGGGGGCCACGGCTCCTGCATTGATGGGAACTGTGTCTGCTCTGCTGGCTACAAAGGCG
AGCACTGTGAGGAAGTTGATTGCTTGGATCCCACCTGCTCCAGCCACGGAGTCTGTGTGAATGGAGAA
TGCCTGTGCAGCCCTGGCTGGGGTGGTCTGAACTGTGAGCTGGCGAGGGTCCAGTGCCCAGACCAGTG
CAGTGGGCATGGCACGTACCTGCCTGACACAGGCCTCTGCAGCTGCGATCCCAACTGGATGGGTCCCG
ACTGCTCTGTTGAAGTGTGCTCAGTAGACTGTGGCACTCACGGCGTCTGCATCGGGGGAGCCTGCCGC
TGTGAAGAGGGCTGGACAGGCGCAGCGTGTGACCAGCGCGTGTGCCACCCCCGCTGCATTGAGCATGG
GACCTGTAAAGATGGCAAATGTGAATGCCGAGAGGGCTGGAATGGTGAACACTGCACCATTGATGGCT
GCCCTGACTTGTGCAACGGTAACGGGAGATGCACACTGGGTCAGAACAGCTGGCAGTGTGTCTGCCAG
ACCGGCTGGAGAGGGCCCGGATGCAACGTTGCCATGGAAACTTCCTGTGCTGATAACAAGGATAATGA
GGGAGATGGCCTGGTGGATTGTTTGGACCCTGACTGCTGCCTGCAGTCAGCCTGTCAGAACAGCCTGC
TCTGCCGGGGGTCCCGGGACCCACTGGACATCATTCAGCAGGGCCAGACGGATTGGCCCGCAGTGAAG
TCCTTCTATGACCGTATCAAGCTCTTGGCAGGCAAGGATAGCACCCACATCATTCCTGGAGAGAACCC
TTTCAACAGCAGCTTGGTTTCTCTCATCCGAGGCCAAGTAGTAACTACAGATGGAACTCCCCTGGTCG
GTGTGAACGTGTCTTTTGTCAAGTACCCAAAATACGGCTACACCATCACCCGCCAGGATGGCACGTTC
GACCTGATCGCAAATGGAGGTGCTTCCTTGACTCTACACTTTGAGCGAGCCCCGTTCATGAGCCAGGA
GCGCACTGTGTGGCTGCCGTGGAACAGCTTTTACGCCATGGACACCCTGGTGATGAAGACCGAGGAGA
ACTCCATCCCCAGCTGTGACCTCAGTGGCTTTGTCCGGCCTGATCCAATCATCATCTCCTCCCCACTG
TCCACCTTCTTTAGTGCTGCCCCTGGGCAGAATCCCATCGTGCCTGAGACCCAGGTTCTTCATGAAGA
AATCGAGCTCCCTGGTTCCAATGTGAAACTTCGCTATCTGAGCTCTAGAACTGCAGGGTACAAGTCAC
TGCTGAAGATCACCATGACCCAGTCCACAGTGCCCCTGAACCTCATTAGGGTTCACCTGATGGTGGCT
GTCGAGGGGCATCTCTTCCAGAAGTCATTCCAGGCTTCTCCCAACCTGGCCTACACCTTCATCTGGGA
CAAGACAGATGCGTATGGCCAAAGGGTGTATGGACTCTCAGATGCTGTTGTGTCTGTCGGGTTTGAAT
ATGAGACCTGTCCCAGTCTAATTCTCTGGGAGAAAAGGACAGCCCTCCTTCAGGGATTCGAGCTGGAC
CCCTCCAACCTCGGTGGCTGGTCCCTAGACAAACACCACATCCTCAATGTTAAAAGTGGAATCCTACA
CAAAGGCACTGGGGAAAACCAGTTCCTGACCCAGCAGCCTGCCATCATCACCAGCATCATGGGCAATG
GTCGCCGCCGGAGCATTTCCTGTCCCAGCTGCAACGGCCTTGCTGAAGGCAACAAGCTGCTGGCCCCA
GTGGCTCTGGCTGTTGGAATCGATGGGAGCCTCTATGTGGGTGACTTCAATTACATCCGACGCATCTT
TCCCTCTCGAAATCTCGAGGGC

| | | | |
|---|---|---|---|
| NOV7f, 308530526 | SEQ ID NO: 124 | 823 aa | MW at 89649.5kD |
| Protein Sequence | | | |

TKLKNSSIDSGEAEVGRRVTQEVPPGVFWRSQIHISQPQFLKFNISLGKDALFGVYIRRGLPPSHAQY
DFMERLDGKEKWSVVESPRERRSIQTLVQNEAVFVQYLDVGLWHLAFYNDGKDKEMVSFNTVVLDSVQ
DCPRNCHGNGECVSGVCHCFPGFLGADCAKAACPVLCSGNGQYSKGTCQCYSGWKGAECDVPMNQCID
PSCGGHGSCIDGNCVCSAGYKGEHCEEVDCLDPTCSSHGVCVNGECLCSPGWGGLNCELARVQCPDQC
SGHGTYLPDTGLCSCDPNWMGPDCSVEVCSVDCGTHGVCIGGACRCEEGWTGAACDQRVCHPRCIEHG
TCKDGKCECREGWNGEHCTIDGCPDLCNGNGRCTLGQNSWQCVCQTGWRGPGCNVAMETSCADNKDNE
GDGLVDCLDPDCCLQSACQNSLLCRGSRDPLDIIQQGQTDWPAVKSFYDRIKLLAGKDSTHIIPGENP
FNSSLVSLIRGQVVTTDGTPLVGVNVSFVKYPKYGYTITRQDGTFDLIANGGASLTLHFERAPFMSQE
RTVWLPWNSFYAMDTLVMKTEENSIPSCDLSGFVRPDPIIISSPLSTFFSAAPGQNPIVPETQVLHEE
IELPGSNVKLRYLSSRTAGYKSLLKITMTQSTVPLNLIRVHLMVAVEGHLFQKSFQASPNLAYTFIWD
KTDAYGQRVYGLSDAVVSVGFEYETCPSLILWEKRTALLQGFELDPSNLGGWSLDKHHILNVKSGILH
KGTGENQFLTQQPAIITSIMGNGRRRSISCPSCNGLAEGNKLLAPVALAVGIDGSLYVGDFNYIRRIF
PSRNLEG

| | | |
|---|---|---|
| NOV7g, 308530589 | SEQ ID NO: 125 | 2284 bp |
| DNA Sequence | ORF Start: at 2 | ORF Stop: end of sequence |

CACCAAGCTTAAAAACAGCAGCATAGACAGTGGTGAAGCAGAAGTTGGTCGGCGGGTAACACAAGAAG
TCCCACCAGGGGTGTTTTGGAGGTCACAAATTCACATCAGTCAGCCCCAGTTCTTAAAGTTCAACATC
TCCCTCGGGAAGGACGCTCTCTTTGGTGTTTACATAAGAAGAGGACTTCCACCATCTCATGCCCAGTA
TGACTTCATGGAACGTCTGGACGGGAAGGAGAAGTGGAGTGTGGTTGAGTCTCCCAGGGAACGCCGGA
GCATACAGACCTTGGTTCAGAATGAAGCCGTGTTTGTGCAGTACCTGGATGTGGGCCTGTGGCATCTG
GCCTTCTACAATGATGGAAAAGACAAAGAGATGGTTTCCTTCAATACTGTTGTCCTAGATTCAGTGCA
GGACTGTCCACGTAACTGCCATGGGAATGGTGAATGTGTGTCCGGGGTGTGTCACTGTTTCCCAGGAT
TTCTAGGAGCAGACTGTGCTAAAGCTGCCTGCCCTGTCCTGTGCAGTGGGAATGGACAATATTCTAAA
GGGACGTGCCAGTGCTACAGCGGCTGGAAAGGTGCAGAGTGCGACGTGCCCATGAATCAGTGCATCGA
TCCTTCCTGCGGGGGCCACGGCTCCTGCATTGATGGGAACTGTGTCTGCTCTGCTGGCTACAAAGGCG
AGCACTGTGAGGAAGTTGATTGCTTGGATCCCACCTGCTCCAGCCACGGAGTCTGTGTGAATGGAGAA
TGCCTGTGCAGCCCTGGCTGGGGTGGTCTGAACTGTGAGCTGGCGAGGGTCCAGTGCCCAGACCAGTG
CAGTGGGCATGGCACGTACCTGCCTGACACGGGCCTCTGCAGCTGCGATCCCAACTGGATGGGTCCCG
ACTGCTCTGTTGATGGCTGCCCTGACTTGTGCAACGGTAACGGGAGATGCACACTGGGTCAGAACAGC
TGGCAGTGTGTCTGCCAGACCGGCTGGAGAGGGCCCGGATGCAACGTTGCCATGGAAACTTCCTGTGC
TGATAACAAGGATAATGAGGGAGATGGCCTGGTGGATTGTTTGGACCCTGACTGCTGCCTGCAGTCAG
CCTGTCAGAACAGCCTGCTCTGCCGGGGGTCCCGGGACCCACTGGACATCATTCAGCAGGGCCAGACG
GATTGGCCCGCAGTGAAGTCCTTCTATGACCGTATCAAGCTCTTGGCAGGCAAGGATAGCACCCACAT
CATTCCTGGAGAGAACCCTTTCAACAGCAGCTTGGTTTCTCTCATCCGAGGCCAAGTAGTAACTACAG
ATGGAACTCCCCTGGTCGGTGTGAACGTGTCTTTTGTCAAGTACCCAAAATACGGCTACACCATCACC
CGCCAGGATGGCACGTTCGACCTGATCGCAAATGGAGGTGCTTCCTTGACTCTACACTTTGAGCGAGC
CCCGTTCATGAGCCAGGAGCGCACTGTGTGGCTGCCGTGGAACAGCTTTTACGCCATGGACACCCTGG
TGATGAAGACCGAGGAGAACTCCATCCCCAGCTGTGACCTCAGTGGCTTTGTCCGGCCTGATCCAATC
ATCATCTCCTCCCCACTGTCCACCTTCTTTAGTGCTGCCCCTGGGCAGAATCCCATCGTGCCTGAGAC
CCAGGTTCTTCATGAAGAAATCGAGCTCCCTGGTTCCAATGTGAAACTTCGCTATCTGAGCTCTAGAA
CTGCAGGGTACAAGTCACTGCTGAAGATCACCATGACCCAGTCCACAGTGCCCCTGAACCTCATTAGG
GTTCACCTGATGGTGGCTGTCGAGGGGCATCTCTTCCAGAAGTCATTCCAGGCTTCTCCCAACCTGGC
CTACACCTTCATCTGGGACAAGACAGATGCGTATGGCCAAAGGGTGTATGGACTCTCAGATGCTGTTG
TGTCTGTCGGGTTTGAATATGAGACCTGTCCCAGTCTAATTCTCTGGGAGAAAAGGACAGCCCTCCTT
CAGGGATTCGAGCTGGACCCCTCCAACCTCGGTGGCTGGTCCCTAGACAAACACCACATCCTCAATGT
TAAAAGTGGAATCCTACACAAAGGCACTGGGGAAAACCAGTTCCTGACCCAGCAGCCTGCCATCATCA
CCAGCATCATGGGCAATGGTCGCCGCCGGAGCATTTCCTGTCCCAGCTGCAACGGCCTTGCTGAAGGC
AACAAGCTGCTGGCCCCAGTGGCTCTGGCTGTTGGAATCGATGGGAGCCTCTATGTGGGTGACTTCAA
TTACATCCGACGCATCTTTCCCTCTCGAAATCTCGAGGGC

| | | | |
|---|---|---|---|
| NOV7g, 308530589 | SEQ ID NO: 126 | 761 aa | MW at 83010.1kD |
| Protein Sequence | | | |

TKLKNSSIDSGEAEVGRRVTQEVPPGVFWRSQIHISQPQFLKFNISLGKDALFGVYIRRGLPPSHAQY
DFMERLDGKEKWSVVESPRERRSIQTLVQNEAVFVQYLDVGLWHLAFYNDGKDKEMVSFNTVVLDSVQ
DCPRNCHGNGECVSGVCHCFPGFLGADCAKAACPVLCSGNGQYSKGTCQCYSGWKGAECDVPMNQCID
PSCGGHGSCIDGNCVCSAGYKGEHCEEVDCLDPTCSSHGVCVNGECLCSPGWGGLNCELARVQCPDQC
SGHGTYLPDTGLCSCDPNWMGPDCSVDGCPDLCNGNGRCTLGQNSWQCVCQTGWRGPGCNVAMETSCA
DNKDNEGDGLVDCLDPDCCLQSACQNSLLCRGSRDPLDIIQQGQTDWPAVKSFYDRIKLLAGKDSTHI
IPGENPFNSSLVSLIRGQVVTTDGTPLVGVNVSFVKYPKYGYTITRQDGTFDLIANGGASLTLHFERA
PFMSQERTVWLPWNSFYAMDTLVMKTEENSIPSCDLSGFVRPDPIIISSPLSTFFSAAPGQNPIVPET
QVLHEEIELPGSNVKLRYLSSRTAGYKSLLKITMTQSTVPLNLIRVHLMVAVEGHLFQKSFQASPNLA
YTFIWDKTDAYGQRVYGLSDAVVSVGFEYETCPSLILWEKRTALLQGFELDPSNLGGWSLDKHHILNV
KSGILHKGTGENQFLTQQPAIITSIMGNGRRRSISCPSCNGLAEGNKLLAPVALAVGIDGSLYVGDFN
YIRRIFPSRNLEG

| | | |
|---|---|---|
| NOV7h, CG50426-01 | SEQ ID NO: 127 | 1431 bp |
| DNA Sequence | ORF Start: ATG at 69 | ORF Stop: TAG at 1212 |

AAAAAAGGCGGGGGGTGGACTTAGCAGTGTAATTTGAGACCGGTGGTAAGGATTGGAGCGAGCTAGAG
ATGCTGCACGCTGCTAACAAGGGAAGGAAGCCTTCAGCTGAGGCAGGTCGTCCCATTCCACCTACATC
CTCGCCTAGTCTCCTCCCATCTGCTCAGCTGCCTAGCTCCCATAATCCTCCACCAGTTAGCTGCCAGA
TGCCATTGCTAGACAGCAACACCTCCCATCAAATCATGGACACCAACCCTGATGAGGAATTCTCCCCC
AATTCATACCTGCTCAGAGCATGCTCAGGGCCCCAGCAAGCCTCCAGCAGTGGCCCTCCGAACCACCA
CAGCCAGTCGACTCTGAGGCCCCCTCTCCCACCCCCTCACAACCACACGCTGTCCCATCACCACTCGT
CCGCCAACTCCCTCAACAGGAACTCACTGACCAATCGGCGGAGTCAGATCCACGCCCCGGCCCCAGCG
CCCAATGACCTGGCCACCACACCAGAGTCCGTTCAGCTTCAGGACAGCTGGGTGCTAAACAGCAACGT
GCCACTGGAGACCCGGCACTTCCTCTTCAAGACCTCCTCGGGGAGCACACCCTTGTTCAGCAGCTCTT
CCCCGGGATACCCTTTGACCTCAGGAACGGTTTACACGCCCCCGCCCCGCCTGCTGCCCAGGAATACT
TTCTCCAGGAAGGCTTTCAAGCTGAAGAAGCCCTCCAAATACTGCAGCTGGAAATGTGCTGCCCTCTC
CGCCATTGCCGCGGCCCTCCTCTTGGCTATTTTGCTGGCGTATTTCATAGTGCCCTGGTCGTTGAAAA
ACAGCAGCATAGACAGTGGTGAAGCAGAAGTTGGTCGGCGGGTAACACAAGAAGTCCCACCAGGGGTG
TTTTGGAGGTCACAAATTCACATCAGTCAGCCCCAGTTCTTAAAGTTCAACATCTCCCTCGGGAAGGA
CGCTCTCTTTGGTGTTTACATAAGAAGAGGACTTCCACCATCTCATGCCCAGTATGACTTCATGGAAC
GTCTGGACGGGAAGGAGAAGTGGAGTGTGGTTGAGTCTCCCAGGGAACGCCGGAGCATACAGACCTTG
GTTCAGAATGAAGCCGTGTTTGTGCAGTACCTGGATGTGGGCCTGTGGCATCTGGCCTTCTACAATGA
TGGAAAAGACAAAGAGATGGTTTCCTTCAATACTGTTGTCCTAGATGGGACCATCTAGTTGCAGAAAA
ACAAGCTCAGGGCGCCCACTGATTTGACATTATGATTCAGTGCAGGACTGTCCACGTAACTGCCATGG
GAATGGTGAANTGTGTGTCCGGGGTGTGTCACTGTTTCCCAGGATTTCTAGGAGCAGACTGTGCTAAA
GACCTTCCTGCCTTGACTTTCTGCAAGACAATCATTAATAAAGCTGCTCTGTAAATACTAAAAAAAAA
ACA

| | | | |
|---|---|---|---|
| NOV7h, CG50426-01 | SEQ ID NO: 128 | 381 aa | MW at 42017.0kD |
| Protein Sequence | | | |

MLHAANKGRKPSAEAGRPIPPTSSPSLLPSAQLPSSHNPPPVSCQMPLLDSNTSHQIMDTNPDEEFSP
NSYLLHACSGPQQASSSGPPNHHSQSTLRPPLPPPHNHTLSHHHSSANSLNRNSLTNRRSQIHAPAPA
PNDLATTPESVQLQDSWVLNSNVPLETRHFLFKTSSGSTPLFSSSSPGYPLTSGTVYTPPPRLLPRNT
FSRKAFKLKKPSKYCSWKCAALSAIAAALLLAILLAYFIVPWSLKNSSIDSGEAEVGRRVTQEVPPGV
FWRSQIHISQPQFLKFNISLGKDALFGVYIRRGLPPSHAQYDFMERLDGKEKWSVVESPRERRSIQTL
VQNEAVFVQYLDVGLWHLAFYNDGKDKEMVSFNTVVLDGTI

| | | |
|---|---|---|
| NOV7i, CG50426-02 | SEQ ID NO: 129 | 1447 bp |
| DNA Sequence | ORF Start: ATG at 277 | ORF Stop: at 1447 |

GCTCCAAAGCGAGCTGGGACCGAAGACTCTAGGCTAAGTTATCTATGTAGATGGTGTCAGGGAGCGAA
GCTACTGACCGAGCTGCTGTTACATCCAGCTTTTTAATTGCCTAAGCGGTCTGGGGCTTGCTTCGTCA
TTTGGCTTTGCTGTGGAGCACTCCTGTAAAGCCAGCTGAATTGTACATCGAAGATCCACCCTTTTGGT
GGCAAAGTGACAAGTGCCAAAACTCAGGCCTGACTTTTCTGAAAACATCAGCATTCTGCCATATCTGG
AATAATGGATGTAAAGGACCGGCGACACCGCTCTTTGACCAGAGGACGCTGTGGCAAAGAGTGTCGCT
ACACAAGCTCCTCTCTGGACAGTGAGGACTGCCGGGTGCCCACACAGAAATCCTACAGCTCCAGTGAG
ACTCTGAAGGCCTATGACCATGACAGCAGGATGCACTATGGAAACCGAGTCACAGACCTCATCCACCG
GGAGTCAGATGAGTTTCCTAGACAAGGAACCAACTTCACCCTTGCCGAACTGGGCATCTGTGAGCCCT
CCCCACACCGAAGCGGCTACTGCTCCGACATGGGGATCCTTCACCAGGGCTACTCCCTTAGCACAGGG
TCTGACGCCGACTCCGACACCGAGGGAGGGATGTCTCCAGAACACGCCATCAGACTGTGGGGCAGAGG
GATAAAATCCAGGCGCAGTTCCGGCCTGTCCAGTCGTGAAAACTCGGCCCTTACCCTGACTGACTCTG
ACAACGAAAACAAATCAGATGATGAGAACGGTCGTCCCATTCCACCTACATCCTCGCCTAGTCTCCTC
CCATCTGCTCAGCTGCCTAGCTCCCATAATCCTCCACCAGTTAGCTGCCAGATGCCATTGCTAGACAG
CAACACCTCCCATCAAATCATGGACACCAACCCTGATGAGGAATTCTCCCCCAATTCATACCTGCTCA
GAGCATGCTCAGGGCCCCAGCAAGCCTCCAGCAGTGGCCCTCCGAACCACCACAGCCAGTCGACTCTG
AGGCCCCCTCTCCCACCCCCTCACAACCACACGCTGTCCCATCACCACTCGTCCGCCAACTCCCTCAA
CAGGAACTCACTGACCAATCGGCGGAGTCAGATCCACGCCCCGGCCCCAGCGCCCAATGACCTGGCCA
CCACACCAGAGTCCGTTCAGCTTCAGGACAGCTGGGTGCTAAACAGCAACGTGCCACTGGAGACCCGG
CACTTCCTCTTCAAGACCTCCTCGGGGAGCACACCCTTGTTCAGCAGCTCTTCCCCGGGATACCCTTT
GACCTCAGGAACGGTTTACACGCCCCCGCCCCGCCTGCTGCCCAGGAATACTTTCTCCAGGAAGGcTT
TCAAGCTGAAGAAGCCCTCCAAATACTGCAGCTGGAAATGTGCTGCCCTCTCCGCCATTGCCGCGGCC
CTCCTCTTGGCTATTTTGC

| | | | |
|---|---|---|---|
| NOV7i, CG50426-02 | SEQ ID NO: 130 | 390 aa | MW at 42705.6kD |
| Protein Sequence | | | |

MDVKDRPHRSLTRGRCGKECRYTSSSLDSEDCRVPTQKSYSSSETLKAYDHDSRMHYGNRVTDLIHRE
SDEFPRQGTNFTLAELGICEPSPHRSGYCSDMGILHQGYSLSTGSDADSDTEGGMSPEHAIRLWGRGI
KSRRSSGLSSRENSALTLTDSDNENKSDDENGRPIPPTSSPSLLPSAQLPSSHNPPPVSCQMPLLDSN
TSHQIMDTNPDEEFSPNSYLLPACSGPQQASSSGPPNHHSQSTLRPPLPPPHNHTLSHHHSSANSLNR
NSLTNRRSQIHAPAPAPNDLATTPESVQLQDSWVLNSNVPLETRHFLFKTSSGSTPLFSSSSPGYPLT
SGTVYTPPPRLLPRNTFSRKAFKLKKPSKYCSWKCAALSAIAAALLLAIL

| | | |
|---|---|---|
| NOV7j, CG50426-03 | SEQ ID NO: 131 | 4888 bp |
| DNA Sequence | ORF Start: ATG at 247 | ORF Stop: TGA at 1840 |

TACTCACTATAGGGCTCGAGCGGCCGCCCGGGCAGGTCCAGCATTAGATGAGTTGACAAAAATGCAGT
TTCAGCTCTGAAGGTCTGAAAGATTCTGCTGCAACTAAAGCTCTGAAGATTCTGCTACAACTATGACA
TCCATTTTCTCCCACTTCAGACAGGATGAATACAAGGTGGCAAAGTGACAAGTGCCAAAACTCAGGCC
TGACTTTCCTGAAAACATCAGCATTCTGCCATATCTGGAATAATGGATGTAAAGGACCGGCGACACCG
CTCTTTGACCAGAGGACGCTGTGGCAAAGAGTGTCGCTACACAAGCTCCTCTCTGGACAGTGAGGACT
GCCGGGTGCCCACACAGAAATCCTACAGCTCCAGTGAGACTCTGAAGGCCTATGACCATGACAGCAGG
ATGCACTATGGAAACCGAGTCACAGACCTCATCCACCGGGAGTCAGATGAGTTTCCTAGACAAGGAAC
CAACTTCACCCTTGCCGAACTGGGCATCTGTGAGCCCTCCCCACACCGAAGCGGCTACTGCTCCGACA
TGGGGATCCTTCACCAGGGCTACTCCCTTAGCACAGGGTCTGACGCCGACTCCGACACCGAGGGAGGG
ATGTCTCCAGAACACGCCATCAGACTGTGGGGCAGAGGGATAAAATCCAGGCGCAGTTCCGGCCTGTC
CAGTCGTGAAAACTCGGCCCTTACCCTGACTGACTCTGACAACGAAAACAAATCAGATGATGAGAACG
GTCGTCCCATTCCACCTACATCCTCGCCTAGTCTCCTCCCATCTGCTCAGCTGCCTAGCTCCCATAAT
CCTCCACCAGTTAGCTGCCAGATGCCATTGCTAGACAGCAACACCTCCCATCAAATCATGGACACCAA
CCCTGATGAGGAATTCTCCCCCAATTCATACCTGCTCAGAGCATGCTCAGGGCCCCAGCAAGCCTCCA
GCAGTGGCCCTCCGAACCACCACAGCCAGTCGACTCTGAGGCCCCCTCTCCCACCCCCTCACAACCAC
ACGCTGTCCCATCACCACTCGTCCGCCAACTCCCTCAACAGGAACTCACTGACCAATCGGCGGAGTCA
GATCCACGCCCCGGCCCCAGCGCCCAATGACCTGGCCACCACACCAGAGTCCGTTCAGCTTCAGGACA
GCTGGGTGCTAAACAGCAACGTGCCACTGGAGACCCGGCACTTCCTCTTCAAGACCTCCTCGGGGAGC
ACACCCTTGTTCAGCAGCTCTTCCCCGGGATACCCTTTGACCTCAGGAACGGTTTACACGCCCCCGCC
CCGCCTGCTGCCCAGGAATACTTTCTCCAGGAAGGCTTTCAAGCTGAAGAAGCCCTCCAAATACTGCA
GCTGGAAATGTGCTGCCCTCTCCGCCATTGCCGCGGCCCTCCTCTTGGCTATTTTGCTGGCGTATTTC
ATAGTGCCCTGGTCGTTGAAAAACAGCAGCATAGACAGTGGTGAAGCAGAAGTTGGTCGGCGGGTAAC
ACAAGAAGTCCCACCAGGGGTGTTTTGGAGGTCACAAATTCACATCAGTCAGCCCCAGTTCTTAAAGT
TCAACATCTCCCTCGGGAAGGACGCTCTCTTTGGTGTTTACATAAGAAGAGGACTTCCACCATCTCAT
GCCCAGTATGACTTCATGGAACGTCTGGACGGGAAGGAGAAGTGGAGTGTGGTTGAGTCTCCCAGGGA
ACGCCGGAGCATACAGACCTTGGTTCAGAATGAAGCCGTGTTTGTGCAGTACCTGGATGTGGGCCTGT
GGCATCTGGCCTTCTACAATGATGGAAAAGACAAAGAGATGGTTTCCTTCAATACTGTTGTCCTAGAT
AGCTGAGGTAGCCGGCTCTGGCCTCGGCTAGCCCAGAAAGGGGCTCCCACAGTGCAGCAGCGGGCTGA
AGGGCTCCTCAAGCGTGGCCAGAGTGGGCGCTGAGGCCGAGGAGGCACCGAGAGCGAGCGAGGGCTGC
AAGGTCTGCCAGCACGCTGTCACTTCTCACTATTTTGATTGGTATTGTGTCCGGAATTGGTGGGTTCT
TGGTCTCACTGACTTCAAGAATGAAGCCACGGACCCTCGCGTTACATCCAGCTTTTTAATTGCCTAAG
CGGTCTGGGGCTTGCTTCGTCATTTGGCTTTGCTGTGGAGCACTCCTGTAAAGCCAGCTGAATTGTAC
ATCGAAGATCCACCCTTTTGGTGGCAAAGTGACAAGTGCCAAAACTCAGGCCTGACTTTTCTGAAAAC
ATCAGCATTCTGCCATATCTGGAATAATGGATGTAAAGGACCGGCGACACCGCTCTTTGACCAGAGGA
CGCTGTGGCAAAGAGTGTCGCTACACAAGCTCCTCTCTGGACAGTGAGGACTGCCGGGTGCCCACACA
GAAATCCTACAGCTCCAGTGAGACTCTGAAGGCCTATGACCATGACAGCAGGATGCACTATGGAAACC
GAGTCACAGACCTCATCCACCGGGAGTCAGATGAGTTTCCTAGACAAGGAACCAACTTCACCCTTGCC
GAACTGGGCATCTGTGAGCCCTCCCCACACCGAAGCGGCTACTGCTCCGACATGGGGATCCTTCACCA
GGGCTACTCCCTTAGCACAGGGTCTGACGCCGACTCCGACACCGAGGGAGGGATGTCTCCAGAACACG
CCATCAGACTGTGGGGCAGAGGGATAAAATCCAGGCGCAGTTCCGGCCTGTCCAGTCGTGAAAACTCG
GCCCTTACCCTGACTGACTCTGACAACGAAAACAAATCAGATGATGAGAACGGTCGTCCCATTCCACC
TACATCCTCGCCTAGTCTCCTCCCATCTGCTCAGCTGCCTAGCTCCCATAATCCTCCACCAGTTAGCT
GCCAGATGCCATTGCTAGACAGCAACACCTCCCATCAAATCATGGACACCAACCCTGATGAGGAATTC
TCCCCCAATTCATACCTGCTCAGAGCATGCTCAGGGCCCCAGCAAGCCTCCAGCAGTGGCCCTCCGAA
CCACCACAGCCAGTCGACTCTGAGGCCCCCTCTCCCACCCCCTCACAACCACACGCTGTCCCATCACC
ACTCGTCCGCCAACTCCCTCAACAGGAACTCACTGACCAATCGGCGGAGTCAGATCCACGCCCCGGCC
CCAGCGCCCAATGACCTGGCCACCACACCAGAGTCCGTTCAGCTTCAGGACAGCTGGGTGCTAAACAG
CAACGTGCCACTGGAGACCCGGCACTTCCTCTTCAAGACCTCCTCGGGGAGCACACCCTTGTTCAGCA
GCTCTTCCCCGGGATACCCTTTGACCTCAGGAACGGTTTACACGCCCCCGCCCCGCCTGCTGCCCAGG
AATACTTTCTCCAGGAAGGCTTTCAAGCTGAAGAAGCCCTCCAAATACTGCAGCTGGAAATGTGCTGC
CCTCTCCGCCATTGCCGCGGCCCTCCTCTTGGCTATTTTGCGCTCCAAAGCGAGCTGGGACCGAAGAC
TCTAGGCTAAGTTATCTATGTAGATGGTGTCAGGGAGCGAAGCTACTGACCGAGCTGCTGTTACATCC
AGCTTTTTAATTGCCTAAGCGGTCTGGGGCTTGCTTCGTCATTTGGCTTTGCTGTGGAGCACTCCTGT
AAAGCCAGCTGAATTGTACATCGAAGATCCACCCTTTTGGTGGCAAAGTGACAAGTGCCAAAACTCAG
GCCTGACTTTTCTGAAAACATCAGCATTCTGCCATATCTGGAATAATGGATGTAAAGGACCGGCGACA
CCGCTCTTTGACCAGAGGACGCTGTGGCAAAGAGTGTCGCTACACAAGCTCCTCTCTGGACAGTGAGG
ACTGCCGGGTGCCCACACAGAAATCCTACAGCTCCAGTGAGACTCTGAAGGCCTATGACCATGACAGC
AGGATGCACTATGGAAACCGAGTCACAGACCTCATCCACCGGGAGTCAGATGAGTTTCCTAGACAAGG
AACCAACTTCACCCTTGCCGAACTGGGCATCTGTGAGCCCTCCCCACACCGAAGCGGCTACTGCTCCG
ACATGGGGATCCTTCACCAGGGCTACTCCCTTAGCACAGGGTCTGACGCCGACTCCGACACCGAGGGA
GGGATGTCTCCAGAACACGCCATCAGACTGTGGGGCAGAGGGATAAAATCCAGGCGCAGTTCCGGCCT
GTCCAGTCGTGAAAACTCGGCCCTTACCCTGACTGACTCTGACAACGAAAACAAATCAGATGATGAGA
ACGGTCGTCCCATTCCACCTACATCCTCGCCTAGTCTCCTCCCATCTGCTCAGCTGCCTAGCTCCCAT
AATCCTCCACCAGTTAGCTGCCAGATGCCATTGCTAGACAGCAACACCTCCCATCAAATCATGGACAC
CAACCCTGATGAGGAATTCTCCCCCAATTCATACCTGCTCAGAGCATGCTCAGGGCCCCAGCAAGCCT
CCAGCAGTGGCCCTCCGAACCACCACAGCCAGTCGACTCTGAGGCCCCCTCTCCCACCCCCTCACAAC
CACACGCTGTCCCATCACCACTCGTCCGCCAACTCCCTCAACAGGAACTCACTGACCAATCGGCGGAG
TCAGATCCACGCCCCGGCCCCAGCGCCCAATGACCTGGCCACCACACCAGAGTCCGTTCAGCTTCAGG
ACAGCTGGGTGCTAAACAGCAACGTGCCACTGGAGACCCGGCACTTCCTCTTCAAGACCTCCTCGGGG
AGCACACCCTTGTTCAGCAGCTCTTCCCCGGGATACCCTTTGACCTCAGGAACGGTTTACACGCCCCC
GCCCCGCCTGCTGCCCAGGAATACTTTCTCCAGGAAGGCTTTCAAGCTGAAGAAGCCCTCCAAATACT
GCAGCTGGAAATGTGCTGCCCTCTCCGCCATTGCCGCGGCCCTCCTCTTGGCTATTTTGC

| | | | |
|---|---|---|---|
| NOV7j, CG50426-03 | SEQ ID NO: 132 | 531 aa | MW at 58938.0kD |
| Protein Sequence | | | |

MDVKDRRHRSLTRGRCGKECRYTSSSLDSEDCRVPTQKSYSSSETLKAYDHDSRMHYGNRVTDLIHRE
SDEFPRQGTNFTLAELGICEPSPHRSGYCSDMGTLHQGYSLSTGSDADSDTEGGMSPEHAIRLWGRGI
KSRRSSGLSSRENSALTLTDSDNENKSDDENGRPIPPTSSPSLLPSAQLPSSHNPPPVSCQMPLLDSN
TSHQIMDTNPDEEFSPNSYLLRACSGPQQASSSGPPNHHSQSTLRPPLPPPHNHTLSHHHSSANSLNR
NSLTNRRSQIHAPAPAPNDLATTPESVQLQDSWVLNSNVPLETRHFLFKTSSGSTPLFSSSSPGYPLT
SGTVYTPPPRLLPRNTFSRKAFKLKKPSKYCSWKCAALSAIAAALLLAILLAYFIVPWSLKNSSIDSG
EAEVGRRVTQEVPPGVFWRSQIHISQPQFLKFNISLGKDALFGVYIRRGLPPSHAQYDFMERLDGKEK
WSVVESPRERRSIQTLVQNEAVFVQYLDVGLWHLAFYNDGKDKEMVSFNTVVLDS

| | | |
|---|---|---|
| NOV7k, CG50426-04 | SEQ ID NO: 133 | 1605 bp |
| DNA Sequence | ORF Start: ATG at 435 | ORF Stop: at 1605 |

AGCTGAGGTAGCCGGCTCTGGCCTCGGCTAGCCCAGAAAGGGGCTCCCACAGTGCAGCAGCGGGCTGA
AGGGCTCCTCAAGCGTGGCCAGAGTGGGCGCTGAGGCCGAGGAGGCACCGAGAGCGAGCGAGGGCTGC
AAGGTCTGCCAGCACGCTGTCACTTCTCACTATTTTGATTGGTATTGTGTCCGGAATTGGTGGGTTCT
TGGTCTCACTGACTTCAAGAATGAAGCCACGGACCCTCGCGTTACATCCAGCTTTTTAATTGCCTAAC
CGGTCTGGGGCTTGCTTCGTCATTTGGCTTTGCTGTGGAGCACTCCTGTAAAGCCAGCTGAATTGTAC
ATCGAAGATCCACCCTTTTGGTGGCAAAGTGACAAGTGCCAAAACTCAGGCCTGACTTTTCTGAAAAC
ATCAGCATTCTGCCATATCTGGAATAATGGATGTAAAGGACCGGCGACACCGCTCTTTGACCAGAGGA
CGCTGTGGCAAAGAGTGTCGCTACACAAGCTCCTCTCTGGACAGTGAGGACTGCCGGGTGCCCACACA
GAAATCCTACAGCTCCAGTGAGACTCTGAAGGCCTATGACCATGACAGCAGGATGCACTATGGAAACC
GAGTCACAGACCTCATCCACCGGGAGTCAGATGAGTTTCCTAGACAAGGAACCAACTTCACCCTTGCC
GAACTGGGCATCTGTGAGCCCTCCCCACACCGAAGCGGCTACTGCTCCGACATGGGGATCCTTCACCA
GGGCTACTCCCTTAGCACAGGGTCTGACGCCGACTCCGACACCGAGGGAGGGATGTCTCCAGAACACG
CCATCAGACTGTGGGGCAGAGGGATAAAATCCAGGCGCAGTTCCGGCCTGTCCAGTCGTGAAAACTCG
GCCCTTACCCTGACTGACTCTGACAACGAAAACAAATCAGATGATGAGAACGGTCGTCCCATTCCACC
TACATCCTCGCCTAGTCTCCTCCCATCTGCTCAGCTGCCTAGCTCCCATAATCCTCCACCAGTTAGCT
GCCAGATGCCATTGCTAGACAGCAACACCTCCCATCAAATCATGGACACCAACCCTGATGAGGAATTC
TCCCCCAATTCATACCTGCTCAGAGCATGCTCAGGGCCCCAGCAAGCCTCCAGCAGTGGCCCTCCGAA
CCACCACAGCCAGTCGACTCTGAGGCCCCCTCTCCCACCCCCTCACAACCACACGCTGTCCCATCACC
ACTCGTCCGCCAACTCCCTCAACAGGAACTCACTGACCAATCGGCGGAGTCAGATCCACGCCCCGGCC
CCAGCGCCCAATGACCTGGCCACCACACCAGAGTCCGTTCAGCTTCAGGACAGCTGGGTGCTAAACAG
CAACGTGCCACTGGAGACCCGGCACTTCCTCTTCAAGACCTCCTCGGGGAGCACACCCTTGTTCAGCA
GCTCTTCCCCGGGATACCCTTTGACCTCAGGAACGGTTTACACGCCCCCGCCCCGCCTGCTGCCCAGG
AATACTTTCTCCAGGAAGGCTTTCAAGCTGAAGAAGCCCTCCAAATACTGCAGCTGGAAATGTGCTGC
CCTCTCCGCCATTGCCGCGGCCCTCCTCTTGGCTATTTTGC

| | | | |
|---|---|---|---|
| NOV7k, CG50426-04 | SEQ ID NO: 134 | 390 aa | MW at 42705.6kD |
| Protein Sequence | | | |

MDVKDRRHRSLTRGRCGKECRYTSSSLDSEDCRVPTQKSYSSSETLKAYDHDSRMHYGNRVTDLIHRE
SDEFPRQGTNFTLAELGICEPSPHRSGYCSDMGILHQGYSLSTGSDADSDTEGGMSPEHAIRLWGRGI
KSRRSSGLSSRENSALTLTDSDNENKSDDENGRPIPPTSSPSLLPSAQLPSSHNPPPVSCQMPLLDSN
TSHQIMDTNPDEEFSPNSYLLRACSGPQQASSSGPPNHHSQSTLRPPLPPPHNHTLSHHHSSANSLNR
NSLTNRRSQIHAPAPAPNDLATTPESVQLQDSWVLNSNVPLETRHFLFKTSSGSTPLFSSSSPGYPLT
SGTVYTPPPRLLPRNTFSRKAFKLKKPSKYCSWKCAALSAIAAALLLAIL

| | | |
|---|---|---|
| NOV7l, CG50426-05 | SEQ ID NO: 135 | 9668 bp |
| DNA Sequence | ORF Start: ATG at 122 | ORF Stop: TAA at 8321 |

GGGAGTTGGAACCAGCATTAGATGAGTTGACAAAAATGCAGTTTCAGGTGGCAAAGTGACAAGTGCCA
AAACTCAGGCCTGACTTTTCTGAAAACATCAGCATTCTGCCATATCTGGAATAATGGATGTAAAGGAC
CGGCGACACCGCTCTTTGACCAGAGGACGCTGTGGCAAAGAGTGTCGCTACACAAGCTCCTCTCTGGA
CAGTGAGGACTGCCGGGTGCCCACACAGAAATCCTACAGCTCCAGTGAGACTCTGAAGGCCTATGACC
ATGACAGCAGGATGCACTATGGAAACCGAGTCACAGACCTCATCCACCGGGAGTCAGATGAGTTTCCT
AGACAAGGAACCAACTTCACCCTTGCCGAACTGGGCATCTGTGAGCCCTCCCCACACCGAAGCGGCTA
CTGCTCCGACATGGGGATCCTTCACCAGGGCTACTCCCTTAGCACAGGGTCTGACGCCGACTCCGACA
CCGAGGGAGGGATGTCTCCAGAACACGCCATCAGACTGTGGGGCAGAGGGATAAAATCCAGGCGCAGT
TCCGGCCTGTCCAGTCGTGAAAACTCGGCCCTTACCCTGACTGACTCTGACAACGAAAACAAATCAGA
TGATGAGAACGGTCGTCCCATTCCACCTACATCCTCGCCTAGTCTCCTCCCATCTGCTCAGCTGCCTA
GCTCCCATAATCCTCCACCAGTTAGCTGCCAGATGCCATTGCTAGACAGCAACACCTCCCATCAAATC
ATGGACACCAACCCTGATGAGGAATTCTCCCCCAATTCATACCTGCTCAGAGCATGCTCAGGGCCCCA
GCAAGCCTCCAGCAGTGGCCCTCCGAACCACCACAGCCAGTCGACTCTGAGGCCCCCTCTCCCACCCC
CTCACAACCACACGCTGTCCCATCACCACTCGTCCGCCAACTCCCTCAACAGGAACTCACTGACCAAT
CGGCGGAGTCAGATCCACGCCCCGGCCCCAGCGCCCAATGACCTGGCCACCACACCAGAGTCCGTTCA
GCTTCAGGACAGCTGGGTGCTAAACAGCAACGTGCCACTGGAGACCCGGCACTTCCTCTTCAAGACCT
CCTCGGGGAGCACACCCTTGTTCAGCAGCTCTTCCCCGGGATACCCTTTGACCTCAGGAACGGTTTAC
ACGCCCCCGCCCCGCCTGCTGCCCAGGAATACTTTCTCCAGGAAGGCTTTCAAGCTGAAGAAGCCCTC
CAAATACTGCAGCTGGAAATGTGCTGCCCTCTCCGCCATTGCCGCGGCCCTCCTCTTGGCTATTTTGC
TGGCGTATTTCATAGTGCCCTGGTCGTTGAAAAACAGCAGCATAGACAGTGGTGAAGCAGAAGTTGGT
CGGCGGGTAACACAAGAAGTCCCACCAGGGGTGTTTTGGAGGTCACAAATTCACATCAGTCAGCCCCA
GTTCTTAAAGTTCAACATCTCCCTCGGGAAGGACGCTCTCTTTGGTGTTTACATAAGAAGAGGACTTC
CACCATCTCATGCCCAGTATGACTTCATGGAACGTCTGGACGGGAAGGAGAAGTGGAGTGTGGTTGAG
TCTCCCAGGGAACGCCGGAGCATACAGACCTTGGTTCAGAATGAAGCCGTGTTTGTGCAGTACCTGGA
TGTGGGCCTGTGGCATCTGGCCTTCTACAATGATGGAAAAGACAAAGAGATGGTTTCCTTCAATACTG
TTGTCCTAGATTCAGTGCAGGACTGTCCACGTAACTGCCATGGGAATGGTGAATGTGTGTCCGGGGTG
TGTCACTGTTTCCCAGGATTTCTAGGAGCAGACTGTGCTAAAGCTGCCTGCCCTGTCCTGTGCAGTGG
GAATGGACAATATTCTAAAGGGACGTGCCAGTGCTACAGCGGCTGGAAAGGTGCAGAGTGCGACGTGC
CCATGAATCAGTGCATCGATCCTTCCTGCGGGGGCCACGGCTCCTGCATTGATGGGAACTGTGTCTGC
TCTGCTGGCTACAAAGGCGAGCACTGTGAGGAAGTTGATTGCTTGGATCCCACCTGCTCCAGCCACGG
AGTCTGTGTGAATGGAGAATGCCTGTGCAGCCCTGGCTGGGGTGGTCTGAACTGTGAGCTGGCGAGGG
TCCAGTGCCCAGACCAGTGCAGTGGGCATGGCACGTACCTGCCTGACACGGGCCTCTGCAGCTGCGAT
CCCAACTGGATGGGTCCCGACTGCTCTGTTGAAGTGTGCTCAGTAGACTGTGGCACTCACGGCGTCTG
CATCGGGGGAGCCTGCCGCTGTGAAGAGGGCTGGACAGGCGCAGCGTGTGACCAGCGCGTGTGCCACC
CCCGCTGCATTGAGCACGGGACCTGTAAAGATGGCAAATGTGAATGCCGAGAGGGCTGGAATGGTGAA
CACTGCACCATTGGTAGGCAAACGGCAGGCACCGAAACAGATGGCTGCCCTGACTTGTGCAACGGTAA
CGGGAGATGCACACTGGGTCAGAACAGCTGGCAGTGTGTCTGCCAGACCGGCTGGAGAGGGCCCGGAT
GCAACGTTGCCATGGAAACTTCCTGTGCTGATAACAAGGATAATGAGGGAGATGGCCTGGTGGATTGT
TTGGACCCTGACTGCTGCCTGCAGTCAGCCTGTCAGAACAGCCTGCTCTGCCGGGGGTCCCGGGACCC
ACTGGACATCATTCAGCAGGGCCAGACGGATTGGCCCGCAGTGAAGTCCTTCTATGACCGTATCAAGC
TCTTGGCAGGCAAGGATAGCACCCACATCATTCCTGGAGAGAACCCTTTCAACAGCAGCTTGGTTTCT
CTCATCCGAGGCCAAGTAGTAACTACAGATGGAACTCCCCTGGTCGGTGTGAACGTGTCTTTTGTCAA
GTACCCAAAATACGGCTACACCATCACCCGCCAGGATGGCACGTTCGACCTGATCGCAAATGGAGGTG
CTTCCTTGACTCTACACTTTGAGCGAGCCCCGTTCATGAGCCAGGAGCGCACTGTGTGGCTGCCGTGG
AACAGCTTTTACGCCATGGACACCCTGGTGATGAAGACCGAGGAGAACTCCATCCCCAGCTGTGACCT
CAGTGGCTTTGTCCGGCCTGATCCAATCATCATCTCCTCCCCACTGTCCACCTTCTTTAGTGCTGCCC
CTGGGCAGAATCCCATCGTGCCTGAGACCCAGGTTCTTCATGAAGAAATCGAGCTCCCTGGTTCCAAT
GTGAAACTTCGCTATCTGAGCTCTAGAACTGCAGGGTACAAGTCACTGCTGAAGATCACCATGACCCA
GTCCACAGTGCCCCTGAACCTCATTAGGGTTCACCTGATGGTGGCTGTCGAGGGGCATCTCTTCCAGA
AGTCATTCCAGGCTTCTCCCAACCTGGCCTCCACCTTCATCTGGGACAAGACAGATGCGTATGGCCAA
AGGGTGTATGGACTCTCAGATGCTGTTGTGTCTGTCGGGTTTGAATATGAGACCTGTCCCAGTCTAAT
TCTCTGGGAGAAAAGGACAGCCCTCCTTCAGGGATTCGAGCTGGACCCCTCCAACCTCGGTGGCTGGT
CCCTAGACAAACACCACATCCTCAATGTTAAAAGTGGAATCCTACACAAAGGCACTGGGGAAAACCAG
TTCCTGACCCAGCAGCCTGCCATCATCACCAGCATCATGGGCAATGGTCGCCGCCGGAGCATTTCCTG
TCCCAGCTGCAACGGCCTTGCTGAAGGCAACAAGCTGCTGGCCCCAGTGGCTCTGGCTGTTGGAATCG
ATGGGAGCCTCTATGTGGGTGACTTCAATTACATCCGACGCATCTTTCCCTCTCGAAATGTGACCAGC
ATCTTGGAGTTACGAAATAAAGAGTTTAAACATAGCAACAACCCAGCACACAAGTACTACTTGGCAGT
GGACCCCGTGTCCGGCTCGCTCTACGTGTCCGACACCAACAGCAGGAGAATCTACCGCGTCAAGTCTC
TGAGTGGAACCAAAGACCTGGCTGGGAATTCGGAAGTTGTGGCAGGGACGGGAGAGCAGTGTCTACCC
TTTGATGAAGCCCGCTGCGGGGATGGAGGGAAGGCCATAGATGCAACCCTGATGAGCCCGAGAGGTAT
TGCAGTAGACAAGAATGGGCTCATGTACTTTGTCGATGCCACCATGATCCGGAAGGTTGACCAGAATG
GAATCATCTCCACCCTGCTGGGCTCCAATGACCTCACTGCCGTCCGGCCGCTGAGCTGTGATTCCAGC
ATGGATGTAGCCCAGGTTCGTCTGGAGTGGCCAACAGACCTTGCTGTCAATCCCATGGATAACTCCTT
GTATGTTCTAGAGAACAATGTCATCCTTCGAATCACCGAGAACCACCAAGTCAGCATCATTGCGGGAC
GCCCCATGCACTGCCAAGTTCCTGGCATTGACTACTCACTCAGCAAACTAGCCATTCACTCTGCCCTG
GAGTCAGCCAGTGCCATTGCCATTTCTCACACTGGGGTCCTCTACATCACTGAGACAGATGAGAAGAA
GATTAACCGTCTACGCCAGGTAACAACCAACGGGGAGATCTGCCTTTTAGCTGGGGCAGCCTCGGACT
GCGACTGCAAAAACGATGTCAATTGCAACTGCTATTCAGGAGATGATGCCTACGCGACTGATGCCATC
TTGAATTCCCCATCATCCTTAGCTGTAGCTCCAGATGGTACCATTTACATTGCAGACCTTGGAAATAT
TCGGATCAGGGCGGTCAGCAAGAACAAGCCTGTTCTTAATGCCTTCAACCAGTATGAGGCTGCATCCC
CCGGAGAGCAGGAGTTATATGTTTTCAACGCTGATGGCATCCACCAATACACTGTGAGCCTGGTGACA
GGGGAGTACTTGTACAATTTCACATATAGTACTGACAATGATGTCACTGAATTGATTGACAATAATGG
GAATTCCCTGAAGATCCGTCGGGACAGCAGTGGCATGCCCCGTCACCTGCTCATGCCTGACAACCAGA
TCATCACCCTCACCGTGGGCACCAATGGAGGCCTCAAAGTCGTGTCCACACAGAACCTGGAGCTTGGT
CTCATGACCTATGATGGCAACACTGGGCTCCTGGCCACCAAGAGCGATGAAACAGGATGGACGACTTT
CTATGACTATGACCACGAAGGCCGCCTGACCAACGTGACGCGCCCCACGGGGGTGGTAACCAGTCTGC
ACCGGGAAATGGAGAAATCTATTACCATTGACATTGAGAACTCCAACCGTGATGATGACGTCACTGTC
ATCACCAACCTCTCTTCAGTAGAGGCCTCCTACACAGTGGTACAAGATCAAGTTCGGAACAGCTACCA
GCTCTGTAATAATGGTACCCTGAGGGTGATGTATGCTAATGGGATGGGTATCAGCTTCCACAGCGAGC
CCCATGTCCTAGCGGGCACCATCACCCCCACCATTGGACGCTGCAACATCTCCCTGCCTATGGAGAAT
GGCTTAAACTCCATTGAGTGGCGCCTAAGAAAGGAACAGATTAAAGGCAAAGTCACCATCTTTGGCAG
GAAGCTCCGGGTCCATGGAAGAAATCTCTTGTCCATTGACTATGATCGAAATATTCGGACTGAAAAGA
TCTATGATGACCACCGGAAGTTCACCCTGAGGATCATTTATGACCAGGTGGGCCGCCCCTTCCTCTGG
CTGCCCAGCAGCGGGCTGGCAGCTGTCAACGTGTCATACTTCTTCAATGGGCGCCTGGCTGGGCTTCA
GCGTGGGGCCATGAGCGAGAGGACAGACATCGACAAGCAAGGCCGCATCGTGTCCCGCATGTTCGCTG
ACGGGAAAGTGTGGAGCTACTCCTACCTTGACAAGTCCATGGTCCTCCTGCTTCAGAGCCAACGTCAG
TATATATTTGAGTATGACTCCTCTGACCGCCTCCTTGCCGTCACCATGCCCAGCGTGGCCCGGCACAG
CATGTCCACACACACCTCCATCGGCTACATCCGTAATATTTACAACCCGCCTGAAAGCAATGCTTCGG
TCATCTTTGACTACAGTGATGACGGCCGCATCCTGAAGACCTCCTTTTTGGGCACCGGACGCCAGGTG
TTCTACAAGTATGGGAAACTCTCCAAGTTATCAGAGATTGTCTACGACAGTACCGCCGTCACCTTCGG
GTATGACGAGACCACTGGTGTCTTGAAGATGGTCAACCTCCAAAGTGGGGGCTTCTCCTGCACCATCA
GGTACCGGAAGATTGGCCCCCTGGTGGACAAGCAGATCTACAGGTTCTCCGAGGAAGGCATGGTCAAT
GCCAGGTTTGACTACACCTATCATGACAACAGCTTCCGCATCGCAAGCATCAAGCCCGTCATAAGTGA
GACTCCCCTCCCCGTTGACCTCTACCGCTATGATGAGATTTCTGGCAAGGTGGAACACTTTGGTAAGT
TTGGAGTCATCTATTATGACATCAACCAGATCATCACCACTGCCGTGATGACCCTCAGCAAACACTTC
GACACCCATGGGCGGATCAAGGAGGTCCAGTATGAGATGTTCCGGTCCCTCATGTACTGGATGACGGT
GCAATATGACAGCATGGGCAGGGTGATCAAGAGGGAGCTAAAACTGGGGCCCTATGCCAATACCACGA
AGTACACCTATGACTACGATGGGGACGGGCAGCTCCAGAGCGTGGCCGTCAATGACCGCCCGACCTGG
CGCTACAGCTATGACCTTAATGGGAATCTCCACTTACTGAACCCAGGCAACAGTGTGCGCCTCATGCC
CTTGCGCTATGACCTCCGGGATCGGATAACCAGACTCGGGGATGTGCAGTACAAAATTGACGACGATG
GCTATCTGTGCCAGAGAGGGTCTGACATCTTCGAATACAATTCCAAGGGCCTCCTAACAAGAGCCTAC
AACAAGGCCAGCGGGTGGAGTGTCCAGTACCGCTATGATGGCGTAGGACGGCGGGCTTCCTACAAGAC
CAACCTGGGCCACCACCTGCAGTACTTCTACTCTGACCTCCACAACCCGACGCGCATCACCCATGTCT
ACAATCACTCCAACTCGGAGATTACCTCACTGTACTACGACCTCCAGGGCCACCTCTTTGCCATGGAG
AGCAGCAGTGGGGAGGAGTACTATGTTGCCTCTGATAACACAGGGACTCCTCTGGCTGTGTTCAGCAT
CAACGGCCTCATGATCAAACAGCTGCAGTACACGGCCTATGGGGAGATTTATTATGACTCCAACCCCG
ACTTCCAGATGGTCATTGGCTTCCATGGGGGACTCTATGACCCCCTGACCAAGCTGGTCCACTTCACT
CAGCGTGATTATGATGTGCTGGCAGGACGATGGACCTCCCCAGACTATACCATGTGGAAAAACGTGGG
CAAGGAGCCGGCCCCCTTTAACCTGTATATGTTCAAGAGCAACAATCCTCTCAGCAGTGAGCTAGATT
TGAAGAACTACGTGACAGATGTGAAAAGCTGGCTTGTGATGTTTGGATTTCAGCTTAGCAACATCATT
CCTGGCTTCCCGAGAGCCAAAATGTATTTCGTGCCTCCTCCCTATGAATTGTCAGAGAGTCAAGCAAG
TGAGAATGGACAGCTCATTACAGGTGTCCAACAGACAACAGAGAGACATAACCAGGCCTTCATGGCTC
TGGAAGGACAGGTCATTACTAAAAAGCTCCACGCCAGCATCCGAGAGAAAGCAGGTCACTGGTTTGCC
ACCACCACGCCCATCATTGGCAAAGGCATCATGTTTGCCATCAAAGAAGGGCGGGTGACCACGGGCGT
GTCCAGCATCGCCAGCGAAGATAGCCGCAAGGTGGCATCTGTGCTGAACAACGCCTACTACCTGGACA
AGATGCACTACAGCATCGAGGGCAAGGACACCCACTACTTTGTGAAGATTGGCTCAGCCGATGGCGAC
CTGGTCACACTAGGCACCACCATCGGCCGCAAGGTGCTAGAGAGCGGGGTGAACGTGACCGTGTCCCA
GCCCACGCTGCTGGTCAACGGCAGGACTCGAAGGTTCACGAACATTGAGTTCCAGTACTCCACGCTGC
TGCTCAGCATCCGCTATGGCCTCACCCCCGACACCCTGGACGAAGAGAAGGCCCGCGTCCTGGACCAG
GCGAGACAGAGGGCCCTGGGCACGGCCTGGGCCAAGGAGCAGCAGAAAGCCAGGGACGGGAGAGAGGG
GAGCCGCCTGTGGACTGAGGGCGAGAAGCAGCAGCTTCTGAGCACCGGGCGCGTGCAAGGGTACGAGG
GATATTACGTGCTTCCCGTGGAGCAATACCCAGAGCTTGCAGACAGTAGCAGCAACATCCAGTTTTTA
AGACAGAATGAGATGGGAAAGAGGTAACAAAATAATCTGCTGCCATTCCTTGTCTGAATGGCTCAGCA
GGAGTAACTGTTATCTCCTCTCCTAAGGAGATGAAGACCTAACAGGGGCACTGCGGCTGCGCTGCTTT
AGGAGACCAAGTGGCAAGAAAGCTCACATTTTTTGAGTTCAAATGCTACTGTCCAAGCGAGAAGTCCC
TCATCCTGAAGTAGACTAAAGCCCGGCTGAAAATTCCGAGGAAAACAAAACAAACGAATGAATGAACA
GACACACACAATGTTCCAAGTTCCCCTAAAATATGACCCACTTGTTCTGGGTCTACGCAGAAAAGAGA
CGCAAAGTGTCCAAAAGGAACAAAAGAACAAAAACGAATAAGCAAAGAAGAAAACAAACAAAAACAAA
ACAAAACAAACACACGGACCGATAAACAAAGAAGCGAAGATAAGAAAGAAGGCCTCATATCCAATTAC
CTCACTCATTCACATGTGAGCGACACGCAGACATCCGCGAGGGCCAGCGTCACCAGACCAGCTGCGGG
ACAAACCACTCAGACTGCTTGTAGGACAAATACTTCTGACATTTTCGTTTAAGCAAATACAGGTGCAT
TTAAAACACGACTTTGGGGGTGATTTGTGTGTAGCGCCTGGGGAGGGGGGATAAAAGAGGAGGAGTGA
GCACTGGAAATACTTTTTAAAGAAAAAAAAACATGAGGGAATAAAAGAAATTCCTATCAAAAATCAAA
GTGAAATAATACCATCCAGCACTTAACTCTCAGGTCCCAACTAAGTCTGGCCTGAGCTAATTTATTTG
AGCGCAGAGTGTAAAATTTAATTCAAAATGGTGGCTATAATCACTACAGATAAATTTCATACTCTTTT
GTCTTTGGAGATTCCATTGTGGACAGTAATACGCAGTTACAGGGTGTAGTCTGTTTAGATTCCGTAGT
TCGTGGGTATCAGTTTCGGTAGAGGTGCAGCATCGTGACACTTTTGCTAACAGGTACCACTTCTGATC
ACCCTGTACATACATGAGCCGAAAGGCACAATCACTGTTTCAGATTTAAAATTATTAGTGTGTTTGTT
TGGTCCAGAAACTGAGACAATCACATGACAGTCACCACGAGGAGAGAAAATTTAAAAAATAAAAATAA
AAACAAAAAAAATTTTAAAAATTAAAAAAACAAAAATAAAGTCTAATAAGAACTTTGGTACAGGAACT
TTTTTGTAATATACATGTATGAATTGTTCATCGAGTTTTTATATTAATTTTAATTTGCTGCTAAGCAA
AGACTAGGGACAGGCAAAGATAATTTATGGCAAAGTGTTTAAATTGTTTATACATAAATAAAGTCTCT
AAAACTCCTGTG

| | | | |
|---|---|---|---|
| NOV7l, CG50426-05 | SEQ ID NO: 136 | 2733 aa | MW at 303423.3kD |
| Protein Sequence | | | |

MDVKDRRHRSLTRGRCGKECRYTSSSLDSEDCRVPTQKSYSSSETLKAYDHDSRMHYGNRVTDLIHRE
SDEFPRQGTNFTLAELGICEPSPHRSGYCSDMGILHQGYSLSTGSDADSDTEGGMSPEHAIRLWGRGI
KSPRSSGLSSRENSALTLTDSDNENKSDDENGRPIPPTSSPSLLPSAQLPSSHNPPPVSCQMPLLDSN
TSHQIMDTNPDEEFSPNSYLLPACSGPQQASSSGPPNHHSQSTLRPPLPPPHNHTLSHHHSSANSLNR
NSLTNRRSQIHAPAPAPNDLATTPESVQLQDSWVLNSNVPLETRHFLFKTSSGSTPLFSSSSPGYPLT
SGTVYTPPPRLLPRNTFSRKAFKLKKPSKYCSWKCAALSAIAAALLLAILLAYFIVPWSLKNSSIDSG
EAEVGRRVTQEVPPGVFWRSQIHISQPQFLKFNISLGKDALFGVYIRRGLPPSHAQYDFMERLDGKEK
WSVVESPRERRSIQTLVQNEAVFVQYLDVGLWHLAFYNDGKDKEMVSFNTVVLDSVQDCPRNCHGNGE
CVSGVCHCFPGFLGADCAKAACPVLCSGNGQYSKGTCQCYSGWKGAECDVPMNQCIDPSCGGHGSCID
GNCVCSAGYKGEHCEEVDCLDPTCSSHGVCVNGECLCSPGWGGLNCELARVQCPDQCSGHGTYLPDTG
LCSCDPNWMGPDCSVEVCSVDCGTHGVCIGGACRCEEGWTGAACDQRVCHPRCIEHGTCKDGKCECRE
GWNGEHCTIGRQTAGTETDGCPDLCNGNGRCTLGQNSWQCVCQTGWRGPGCNVAMETSCADNKDNEGD
GLVDCLDPDCCLQSACQNSLLCRGSRDPLDIIQQGQTDWPAVKSFYDRIKLLAGKDSTHIIPGENPFN
SSLVSLIRGQVVITDGTPLVGVNVSFVKYPKYGYTITRQDGTFDLIANGGASLTLHFERAPFMSQERT
VWLPWNSFYAMDTLVMKTEENSIPSCDLSGFVRPDPIIISSPLSTFFSAAPGQNPTVPETQVLHEEIE
LPGSNVKLRYLSSRTAGYKSLLKITMTQSTVPLNLIRVHLMVAVEGHLFQKSFQASPNLASTFIWDKT
DAYGQRVYGLSDAVVSVGFEYETCPSLILWEKRTALLQGFELDPSNLGGWSLDKHHILNVKSGILHKG
TGENQFLTQQPAIITSIMGNGRRRSISCPSCNGLAEGNKLLAPVALAVGIDGSLYVGDFNYIRRIFPS
RNVTSILELRNKEFKHSNNPAHKYYLAVDPVSGSLYVSDTNSRRIYRVKSLSGTKDLAGNSEVVAGTG
EQCLPFDEARCGDGGKAIDATLMSPRGIAVDKNGLMYFVDATMIRKVDQNGIISTLLGSNDLTAVRPL
SCDSSMDVAQVRLEWPTDLAVNPMDNSLYVLENNVILRITENHQVSIIAGRPMHCQVPGIDYSLSKLA
IHSALESASAIAISHTGVLYITETDEKKINRLRQVTTNGEICLLAGAASDCDCKNDVNCNCYSGDDAY
ATDAILNSPSSLAVAPDGTIYIADLGNIRIRAVSKNKPVLNAFNQYEAASPGEQELYVFNADGIHQYT
VSLVTGEYLYNFTYSTDNDVTELIDNNGNSLKIRRDSSGMPRHLLMPDNQIITLTVGTNGGLKVVSTQ
NLELGLMTYDGNTGLLATKSDETGWTTFYDYDHEGRLTNVTRPTGVVTSLHREMEKSITIDIENSNRD
DDVTVITNLSSVEASYTVVQDQVFNSYQLCNNGTLRVMYANGMGISFHSEPHVLAGTITPTIGRCNIS
LPMENGLNSIEWRLRKEQIKGKVTIFGRKLRVHGRNLLSIDYDRNIRTEKIYDDHRKFTLRIIYDQVG
RPFLWLPSSGLAAVNVSYFFNGRLAGLQRGAMSERTDIDKQGRIVSRMFADGKVWSYSYLDKSMVLLL
QSQRQYIFEYDSSDRLLAVTMPSVARHSMSTHTSIGYIRNIYNPPESNASVIFDYSDDGRILKTSFLG
TGRQVFYKYGKLSKLSEIVYDSTAVTFGYDETTGVLKMVNLQSGGFSCTIRYRKIGPLVDKQIYRFSE
EGMVNARFDYTYHDNSFRIASIKPVISETPLPVDLYRYDEISGKVEHFGKFGVIYYDINQIITTAVMT
LSKHFDTHGRIKEVQYEMFRSLMYWMTVQYDSMGRVIKRELKLGPYANTTKYTYDYDGDGQLQSVAVN
DRPTWRYSYDLNGNLHLLNPGNSVRLMPLRYDLRDRITRLGDVQYKIDDDGYLCQRGSDIFEYNSKGL
LTRAYNKASGWSVQYRYDGVGRRASYKTNLGHHLQYFYSDLHNPTRITHVYNHSNSEITSLYYDLQGH
LFAMESSSGEEYYVASDNTGTPLAVFSINGLMIKQLQYTAYGEIYYDSNPDFQMVIGFHGGLYDPLTK
LVHFTQRDYDVLAGRWTSPDYTMWKNVGKEPAPFNLYMFKSNNPLSSELDLKNYVTDVKSWLVMFGFQ
LSNIIPGFPRAKMYFVPPPYELSESQASENGQLITGVQQTTERHNQAFMALEGQVITKKLHASIREKA
GHWFATTTPIIGKGIMFAIKEGRVTTGVSSIASEDSRKVASVLNNAYYLDKMHYSIEGKDTHYFVKIG
SADGDLVTLGTTIGRKVLESGVNVTVSQPTLLVNGRTRRFTNIEFQYSTLLLSIRYGLTPDTLDEEKA
RVLDQARQRALGTAWAKEQQKARDGREGSRLWTEGEKQQLLSTGRVQGYEGYYVLPVEQYPELADSSS
NIQFLRQNEMGKR

| | | |
|---|---|---|
| NOV7m, CG50426-06 | SEQ ID NO: 137 | 1920 bp |
| DNA Sequence | ORF Start: at 180 | ORF Stop: at 1920 |

CCAGCATTAGATGAGTTGACAAAAATGCAGTTTCAGCTCTGAAGGTCTGAAAGATTCTGCTGCAACTA
AAGCTCTGAAGATTCTGCTACAACTATGACATCCATTTTCTCCCACTTCAGACAGGATGAATACAAGC
TGGCAAAGTGACAAGTGCCAAAACTCAGGCCTGACTTTCCTGAAAACATCAGCATTCTGCCATATCTG
GAATAATGGATGTAAAGGACCGGCGACACCGCTCTTTGACCAGAGGACGCTGTGGCAAAGAGTGTCGC
TACACAAGCTCCTCTCTGGACAGTGAGGACTGCCGGGTGCCCACACAGAAATCCTACAGCTCCAGTGA
GACTCTGAAGGCCTATGACCATGACAGCAGGATGCACTATGGAAACCGAGTCACAGACCTCATCCACC
GGGAGTCAGATGAGTTTCCTAGACAAGGAACCAACTTCACCCTTGCCGAACTGGGCATCTGTGAGCCC
TCCCCACACCGAAGCGGCTACTGCTCCGACATGGGGATCCTTCACCAGGGCTACTCCCTTAGCACAGG
GTCTGACGCCGACTCCGACACCGAGGGAGGGATGTCTCCAGAACACGCCATCAGACTGTGGGGCAGAG
GGATAAAATCCAGGCGCAGTTCCGGCCTGTCCAGTCGTGAAAACTCGGCCCTTACCCTGACTGACTCT
GACAACGAAAACAAATCAGATGATGAGAACGGTCGTCCCATTCCACCTACATCCTCGCCTAGTCTCCT
CCCATCTGCTCAGCTGCCTAGCTCCCATAATCCTCCACCAGTTAGCTGCCAGATGCCATTGCTAGACA
GCAACACCTCCCATCAAATCATGGACACCAACCCTGATGAGGAATTCTCCCCCAATTCATACCTGCTC
AGAGCATGCTCAGGGCCCCAGCAAGCCTCCAGCAGTGGCCCTCCGAACCACCACAGCCAGTCGACTCT
GAGGCCCCCTCTCCCACCCCCTCACAACCACACGCTGTCCCATCACCACTCGTCCGCCAACTCCCTCA
ACAGGAACTCACTGACCAATCGGCGGAGTCAGATCCACGCCCCGGCCCCAGCGCCCAATGACCTGGCC
ACCACACCAGAGTCCGTTCAGCTTCAGGACAGCTGGGTGCTAAACAGCAACGTGCCACTGGAGACCCG
GCACTTCCTCTTCAAGACCTCCTCGGGGAGCACACCCTTGTTCAGCAGCTCTTCCCCGGGATACCCTT
TGACCTCAGGAACGGTTTACACGCCCCCGCCCCGCCTGCTGCCCAGGAATACTTTCTCCAGGAAGGCT
TTCAAGCTGAAGAAGCCCTCCAAATACTGCAGCTGGAAATGTGCTGCCCTCTCCGCCATTGCCGCGGC
CCTCCTCTTGGCTATTTTGCTGGCGTATTTCATAGTGCCCTGGTCGTTGAAAAACAGCAGCATAGACA
GTGGTGAAGCAGAAGTTGGTCGGCGGGTAACACAAGAAGTCCCACCAGGGGTGTTTTGGAGGTCACAA
ATTCACATCAGTCAGCCCCAGTTCTTAAAGTTCAACATCTCCCTCGGGAAGGACGCTCTCTTTGGTGT
TTACATAAGAAGAGGACTTCCACCATCTCATGCCCAGTATGACTTCATGGAACGTCTGGACGGGAAGG
AGAAGTGGAGTGTGGTTGAGTCTCCCAGGGAACGCCGGAGCATACAGACCTTGGTTCAGAATGAAGCC
GTGTTTGTGCAGTACCTGGATGTGGGCCTGTGGCATCTGGCCTTCTACAATGATGGAAAAGACAAAGA
GATGGTTTCCTTCAATACTGTTGTCCTAGATTCAGTGCAGGACTGTCCACGTAACTGCCATGGGAATG
GTGAATGTGTGTCCGGGGTGTGTCACTGTTTCCCAGGATTTCTAGGAGCAGACTGTGCTAAAGGTATG
TGCCGCCACTTCCCTG

| | | | |
|---|---|---|---|
| NOV7m, CG50426-06 | SEQ ID NO: 138 | 580 aa | MW at 64132.9kD |
| Protein Sequence | | | |

KHQHSAISGIMDVKDRRHRSLTRGRCGKECRYTSSSLDSEDCRVPTQKSYSSSETLKAYDHDSRMHYG
NRVTDLIHRESDEFPRQGTNFTLAELGICEPSPHRSGYCSDMGILHQGYSLSTGSDADSDTEGGMSPE
HAIRLWGRGIKSRRSSGLSSRENSALTLTDSDNENKSDDENGRPIPPTSSPSLLPSAQLPSSHNPPPV
SCQMPLLDSNTSHQIMDTNPDEEFSPNSYLLRACSGPQQASSSGPPNHHSQSTLRPPLPPPHNHTLSH
HHSSANSLNRNSLTNRRSQIHAPAPAPNDLATTPESVQLQDSWVLNSNVPLETRHFLFKTSSGSTPLF
SSSSPGYPLTSGTVYTPPPRLLPRNTFSRKAFKLKKPSKYCSWKCAALSAIAAALLLAILLAYFIVPW
SLKNSSIDSGEAEVGRRVTQEVPPGVFWRSQIHISQPQFLKFNISLGKDALFGVYIRRGLPPSHAQYD
FMERLDGKEKWSVVESPRERRSIQTLVQNEAVFVQYLDVGLWHLAFYNDGKDKEMVSFNTVVLDSVQD
CPRNCHGNGECVSGVCHCFPGFLGADCAKGMCRHFP

| | | |
|---|---|---|
| NOV7n, CG50426-07 | SEQ ID NO: 139 | 1447 bp |
| DNA Sequence | ORF Start: at 247 | ORF Stop: at 1447 |

GCTCCAAAGCGAGCTGGGACCGAAGACTCTAGGCTAAGTTATCTATGTAGATGGTGTCAGGGAGCGAA
GCTACTGACCGAGCTGCTGTTACATCCAGCTTTTTAATTGCCTAAGCGGTCTGGGGCTTGCTTCGTCA
TTTGGCTTTGCTGTGGAGCACTCCTGTAAAGCCAGCTGAATTGTACATCGAAGATCCACCCTTTTGGT
GGCAAAGTGACAAGTGCCAAAACTCAGGCCTGACTTTTCTGAAAACATCAGCATTCTGCCATATCTGG
AATAATGGATGTAAAGGACCGGCGACACCGCTCTTTGACCAGAGGACGCTGTGGCAAAQAGTGTCGCT
ACACAAGCTCCTCTCTGGACAGTGAGGACTGCCGGGTGCCCACACAGAAATCCTACAGCTCCAGTGAG
ACTCTGAAGGCCTATGACCATGACAGCAGGATGCACTATGGAAACCGAGTCACAGACCTCATCCACCG
GGAGTCAGATGAGTTTCCTAGACAAGGAACCAACTTCACCCTTGCCGAACTGGGCATCTGTGAGCCCT
CCCCACACCGAAGCGGCTACTGCTCCGACATGGGGATCCTTCACCAGGGCTACTCCCTTAGCACAGGG
TCTGACGCCGACTCCGACACCGAGGGAGGGATGTCTCCAGAACACGCCATCAGACTGTGGGGCAGAGG
GATAAAATCCAGGCGCAGTTCCGGCCTGTCCAGTCGTGAAAACTCGGCCCTTACCCTGACTGACTCTG
ACAACGAAAACAAATCAGATGATGAGAACGGTCGTCCCATTCCACCTACATCCTCGCCTAGTCTCCTC
CCATCTGCTCAGCTGCCTAGCTCCCATAATCCTCCACCAGTTAGCTGCCAGATGCCATTGCTAGACAG
CAACACCTCCCATCAAATCATGGACACCAACCCTGATGAGGAATTCTCCCCCAATTCATACCTGCTCA
GAGCATGCTCAGGGCCCCAGCAAGCCTCCAGCAGTGGCCCTCCGAACCACCACAGCCAGTCGACTCTG
AGGCCCCCTCTCCCACCCCCTCACAACCACACGCTGTCCCATCACCACTCGTCCGCCAACTCCCTCAA
CAGGAACTCACTGACCAATCGGCGGAGTCAGATCCACGCCCCGGCCCCAGCGCCCAATGACCTGGCCA
CCACACCAGAGTCCGTTCAGCTTCAGGACAGCTGGGTGCTAAACAGCAACGTGCCACTGGAGACCCGG
CACTTCCTCTTCAAGACCTCCTCGGGGAGCACACCCTTGTTCAGCAGCTCTTCCCCGGGATACCCTTT
GACCTCAGGAACGGTTTACACGCCCCCGCCCCGCCTGCTGCCCAGGAATACTTTCTCCAGGAAGGCTT
TCAAGCTGAAGAAGCCCTCCAAATACTGCAGCTGGAAATGTGCTGCCCTCTCCGCCATTGCCGCGGCC
CTCCTCTTGGCTATTTTGC

| | | | |
|---|---|---|---|
| NOV7n, CG50426-07 | SEQ ID NO: 140 | 400 aa | MW at 43764.8kD |
| Protein Sequence | | | |

KHQHSAISGIMDVKDRRHRSLTRGRCGKECRYTSSSLDSEDCRVPTQKSYSSSETLKAYDHDSRNHYG
NRVTDLIHRESDEFPRQGTNFTLAELGICEPSPHRSGYCSDMGILHQGYSLSTGSDADSDTEGGMSPE
HAIRLWGRGIKSRRSSGLSSRENSALTLTDSDNENKSDDENGRPIPPTSSPSLLPSAQLPSSHNPPPV
SCQMPLLDSNTSHQIMDTNPDEEFSPNSYLLEACSGPQQASSSGPPNHHSQSTLRPPLPPPHNHTLSH
HHSSANSLNRNSLTNRRSQIHAPAPAPNDLATTPESVQLQDSWVLNSNVPLETRHFLFKTSSGSTPLF
SSSSPGYPLTSGTVYTPPPRLLPRNTFSRKAFKLKKPSKYCSWKCAALSAIAAALLLAIL

| | | |
|---|---|---|
| NOV7o, CG50426-08 | SEQ ID NO: 141 | 9823 bp |
| DNA Sequence | ORF Start: ATG at 277 | ORF Stop: TAA at 8476 |

GCTCCAAAGCGAGCTGGGACCGAAGACTCTAGGCTAAGTTATCTATGTAGATGGTGTCAGGGAGCGAA
GCTACTGACCGAGCTGCTGTTACATCCAGCTTTTTAATTGCCTAAGCGGTCTGGGGCTTGCTTCGTCA
TTTGGCTTTGCTGTGGAGCACTCCTGTAAAGCCAGCTGAATTGTACATCGAAGATCCACCCTTTTGGT
GGCAAAGTGACAAGTGCCAAAACTCAGGCCTGACTTTTCTGAAAACATCAGCATTCTGCCATATCTGG
AATAATGGATGTAAAGGACCGGCGACACCGCTCTTTGACCAGAGGACGCTGTGGCAAAGAGTGTCGCT
ACACAAGCTCCTCTCTGGACAGTGAGGACTGCCGGGTGCCCACACAGAAATCCTACAGCTCCAGTGAG
ACTCTGAAGGCCTATGACCATGACAGCAGGATGCACTATGGAAACCGAGTCACAGACCTCATCCACCG
GGAGTCAGATGAGTTTCCTAGACAAGGAACCAACTTCACCCTTGCCGAACTGGGCATCTGTGAGCCCT
CCCCACACCGAAGCGGCTACTGCTCCGACATGGGGATCCTTCACCAGGGCTACTCCCTTAGCACAGGG
TCTGACGCCGACTCCGACACCGAGGGAGGGATGTCTCCAGAACACGCCATCAGACTGTGGGGCAGAGG
GATAAAATCCAGGCGCAGTTCCGGCCTGTCCAGTCGTGAAAACTCGGCCCTTACCCTGACTGACTCTG
ACAACGAAAACAAATCAGATGATGAGAACGGTCGTCCCATTCCACCTACATCCTCGCCTAGTCTCCTC
CCATCTGCTCAGCTGCCTAGCTCCCATAATCCTCCACCAGTTAGCTGCCAGATGCCATTGCTAGACAG
CAACACCTCCCATCAAATCATGGACACCAACCCTGATGAGGAATTCTCCCCCAATTCATACCTGCTCA
GAGCATGCTCAGGGCCCCAGCAAGCCTCCAGCAGTGGCCCTCCGAACCACCACAGCCAGTCGACTCTG
AGGCCCCCTCTCCCACCCCCTCACAACCACACGCTGTCCCATCACCACTCGTCCGCCAACTCCCTCAA
CAGGAACTCACTGACCAATCGGCGGAGTCAGATCCACGCCCCGGCCCCAGCGCCCAATGACCTGGCCA
CCACACCAGAGTCCGTTCAGCTTCAGGACAGCTGGGTGCTAAACAGCAACGTGCCACTGGAGACCCGG
CACTTCCTCTTCAAGACCTCCTCGGGGAGCACACCCTTGTTCAGCAGCTCTTCCCCGGGATACCCTTT
GACCTCAGGAACGGTTTACACGCCCCCGCCCCGCCTGCTGCCCAGGAATACTTTCTCCAGGAAGGCTT
TCAAGCTGAAGAAGCCCTCCAAATACTGCAGCTGGAAATGTGCTGCCCTCTCCGCCATTGCCGCGGCC
CTCCTCTTGGCTATTTTGCTGGCGTATTTCATAGTGCCCTGGTCGTTGAAAAACAGCAGCATAGACAG
TGGTGAAGCAGAAGTTGGTCGGCGGGTAACACAAGAAGTCCCACCAGGGGTGTTTTGGAGGTCACAAA
TTCACATCAGTCAGCCCCAGTTCTTAAAGTTCAACATCTCCCTCGGGAAGGACGCTCTCTTTGGTGTT
TACATAAGAAGAGGACTTCCACCATCTCATGCCCAGTATGACTTCATGGAACGTCTGGACGGGAAGGA
GAAGTGGAGTGTGGTTGAGTCTCCCAGGGAACGCCGGAGCATACAGACCTTGGTTCAGAATGAAGCCG
TGTTTGTGCAGTACCTGGATGTGGGCCTGTGGCATCTGGCCTTCTACAATGATGGAAAAGACAAAGAG
ATGGTTTCCTTCAATACTGTTGTCCTAGATTCAGTGCAGGACTGTCCACGTAACTGCCATGGGAATGG
TGAATGTGTGTCCGGGGTGTGTCACTGTTTCCCAGGATTTCTAGGAGCAGACTGTGCTAAAGCTGCCT
GCCCTGTCCTGTGCAGTGGGAATGGACAATATTCTAAAGGGACGTGCCAGTGCTACAGCGGCTGGAAA
GGTGCAGAGTGCGACGTGCCCATGAATCAGTGCATCGATCCTTCCTGCGGGGGCCACGGCTCCTGCAT
TGATGGGAACTGTGTCTGCTCTGCTGGCTACAAAGGCGAGCACTGTGAGGAAGTTGATTGCTTGGATC
CCACCTGCTCCAGCCACGGAGTCTGTGTGAATGGAGAATGCCTGTGCAGCCCTGGCTGGGGTGGTCTG
AACTGTGAGCTGGCGAGGGTCCAGTGCCCAGACCAGTGCAGTGGGCATGGCACGTACCTGCCTGACAC
GGGCCTCTGCAGCTGCGATCCCAACTGGATGGGTCCCGACTGCTCTGTTGAAGTGTGCTCAGTAGACT
GTGGCACTCACGGCGTCTGCATCGGGGGAGCCTGCCGCTGTGAAGAGGGCTGGACAGGCGCAGCGTGT
GACCAGCGCGTGTGCCACCCCCGCTGCATTGAGCACGGGACCTGTAAAGATGGCAAATGTGAATGCCG
AGAGGGCTGGAATGGTGAACACTGCACCATTGGTAGGCAAACGGCAGGCACCGAAACAGATGGCTGCC
CTGACTTGTGCAACGGTAACGGGAGATGCACACTGGGTCAGAACAGCTGGCAGTGTGTCTGCCAGACC
GGCTGGAGAGGGCCCGGATGCAACGTTGCCATGGAAACTTCCTGTGCTGATAACAAGGATAATGAGGG
AGATGGCCTGGTGGATTGTTTGGACCCTGACTGCTGCCTGCAGTCAGCCTGTCAGAACAGCCTGCTCT
GCCGGGGGTCCCGGGACCCACTGGACATCATTCAGCAGGGCCAGACGGATTGGCCCGCAGTGAAGTCC
TTCTATGACCGTATCAAGCTCTTGGCAGGCAAGGATAGCACCCACATCATTCCTGGAGAGAACCCTTT
CAACAGCAGCTTGGTTTCTCTCATCCGAGGCCAAGTAGTAACTACAGATGGAACTCCCCTGGTCGGTG
TGAACGTGTCTTTTGTCAAGTACCCAAAATACGGCTACACCATCACCCGCCAGGATGGCACGTTCGAC
CTGATCGCAAATGGAGGTGCTTCCTTGACTCTACACTTTGAGCGAGCCCCGTTCATGAGCCAGGAGCG
CACTGTGTGGCTGCCGTGGAACAGCTTTTACGCCATGGACACCCTGGTGATGAAGACCGAGGAGAACT
CCATCCCCAGCTGTGACCTCAGTGGCTTTGTCCGGCCTGATCCAATCATCATCTCCTCCCCACTGTCC
ACCTTCTTTAGTGCTGCCCCTGGGCAGAATCCCATCGTGCCTGAGACCCAGGTTCTTCATGAAGAAAT
CGAGCTCCCTGGTTCCAATGTGAAACTTCGCTATCTGAGCTCTAGAACTGCAGGGTACAAGTCACTGC
TGAAGATCACCATGACCCAGTCCACAGTGCCCCTGAACCTCATTAGGGTTCACCTGATGGTGGCTGTC
GAGGGGCATCTCTTCCAGAAGTCATTCCAGGCTTCTCCCAACCTGGCCTCCACCTTCATCTGGGACAA
GACAGATGCGTATGGCCAAAGGGTGTATGGACTCTCAGATGCTGTTGTGTCTGTCGGGTTTGAATATG
AGACCTGTCCCAGTCTAATTCTCTGGGAGAAAAGGACAGCCCTCCTTCAGGGATTCGAGCTGGACCCC
TCCAACCTCGGTGGCTGGTCCCTAGACAAACACCACATCCTCAATGTTAAAAGTGGAATCCTACACAA
AGGCACTGGGGAAAACCAGTTCCTGACCCAGCAGCCTGCCATCATCACCAGCATCATGGGCAATGGTC
GCCGCCGGAGCATTTCCTGTCCCAGCTGCAACGGCCTTGCTGAAGGCAACAAGCTGCTGGCCCCAGTG
GCTCTGGCTGTTGGAATCGATGGGAGCCTCTATGTGGGTGACTTCAATTACATCCGACGCATCTTTCC
CTCTCGAAATGTGACCAGCATCTTGGAGTTACGAAATAAAGAGTTTAAACATAGCAACAACCCAGCAC
ACAAGTACTACTTGGCAGTGGACCCCGTGTCCGGCTCGCTCTACGTGTCCGACACCAACAGCAGGAGA
ATCTACCGCGTCAAGTCTCTGAGTGGAACCAAAGACCTGGCTGGGAATTCGGAAGTTGTGGCAGGGAC
GGGAGAGCAGTGTCTACCCTTTGATGAAGCCCGCTGCGGGGATGGAGGGAAGGCCATAGATGCAACCC
TGATGAGCCCGAGAGGTATTGCAGTAGACAAGAATGGGCTCATGTACTTTGTCGATGCCACCATGATC
CGGAAGGTTGACCAGAATGGAATCATCTCCACCCTGCTGGGCTCCAATGACCTCACTGCCGTCCGGCC
GCTGAGCTGTGATTCCAGCATGGATGTAGCCCAGGTTCGTCTGGAGTGGCCAACAGACCTTGCTGTCA
ATCCCATGGATAACTCCTTGTATGTTCTAGAGAACAATGTCATCCTTCGAATCACCGAGAACCACCAA
GTCAGCATCATTGCGGGACGCCCCATGCACTGCCAAGTTCCTGGCATTGACTACTCACTCAGCAAACT
AGCCATTCACTCTGCCCTGGAGTCAGCCAGTGCCATTGCCATTTCTCACACTGGGGTCCTCTACATCA
CTGAGACAGATGAGAAGAAGATTAACCGTCTACGCCAGGTAACAACCAACGGGGAGATCTGCCTTTTA
GCTGGGGCAGCCTCGGACTGCGACTGCAAAAACGATGTCAATTGCAACTGCTATTCAGGAGATGATGC
CTACGCGACTGATGCCATCTTGAATTCCCCATCATCCTTAGCTGTAGCTCCAGATGGTACCATTTACA
TTGCAGACCTTGGAAATATTCGGATCAGGGCGGTCAGCAAGAACAAGCCTGTTCTTAATGCCTTCAAC
CAGTATGAGGCTGCATCCCCCGGAGAGCAGGAGTTATATGTTTTCAACGCTGATGGCATCCACCAATA
CACTGTGAGCCTGGTGACAGGGGAGTACTTGTACAATTTCACATATAGTACTGACAATGATGTCACTG
AATTGATTGACAATAATGGGAATTCCCTGAAGATCCGTCGGGACAGCAGTGGCATGCCCCGTCACCTG
CTCATGCCTGACAACCAGATCATCACCCTCACCGTGGGCACCAATGGAGGCCTCAAAGTCGTGTCCAC
ACAGAACCTGGAGCTTGGTCTCATGACCTATGATGGCAACACTGGGCTCCTGGCCACCAAGAGCGATG
AAACAGGATGGACGACTTTCTATGACTATGACCACGAAGGCCGCCTGACCAACGTGACGCGCCCCACG
GGGGTGGTAACCAGTCTGCACCGGGAAATGGAGAAATCTATTACCATTGACATTGAGAACTCCAACCG
TGATGATGACGTCACTGTCATCACCAACCTCTCTTCAGTAGAGGCCTCCTACACAGTGGTACAAGATC
AAGTTCGGAACAGCTACCAGCTCTGTAATAATGGTACCCTGAGGGTGATGTATGCTAATGGGATGGGT
ATCAGCTTCCACAGCGAGCCCCATGTCCTAGCGGGCACCATCACCCCCACCATTGGACGCTGCAACAT
CTCCCTGCCTATGGAGAATGGCTTAAACTCCATTGAGTGGCGCCTAAGAAAGGAACAGATTAAAGGCA
AAGTCACCATCTTTGGCAGGAAGCTCCGGGTCCATGGAAGAAATCTCTTGTCCATTGACTATGATCGA
AATATTCGGACTGAAAAGATCTATGATGACCACCGGAAGTTCACCCTGAGGATCATTTATGACCAGGT
GGGCCGCCCCTTCCTCTGGCTGCCCAGCAGCGGGCTGGCAGCTGTCAACGTGTCATACTTCTTCAATG
GGCGCCTGGCTGGGCTTCAGCGTGGGGCCATGAGCGAGAGGACAGACATCGACAAGCAAGGCCGCATC
GTGTCCCGCATGTTCGCTGACGGGAAAGTGTGGAGCTACTCCTACCTTGACAAGTCCATGGTCCTCCT
GCTTCAGAGCCAACGTCAGTATATATTTGAGTATGACTCCTCTGACCGCCTCCTTGCCGTCACCATGC
CCAGCGTGGCCCGGCACAGCATGTCCACACACACCTCCATCGGCTACATCCGTAATATTTACAACCCG
CCTGAAAGCAATGCTTCGGTCATCTTTGACTACAGTGATGACGGCCGCATCCTGAAGACCTCCTTTTT
GGGCACCGGACGCCAGGTGTTCTACAAGTATGGGAAACTCTCCAAGTTATCAGAGATTGTCTACGACA
GTACCGCCGTCACCTTCGGGTATGACGAGACCACTGGTGTCTTGAAGATGGTCAACCTCCAAAGTGGG
GGCTTCTCCTGCACCATCAGGTACCGGAAGATTGGCCCCCTGGTGGACAAGCAGATCTACAGGTTCTC
CGAGGAAGGCATGGTCAATGCCAGGTTTGACTACACCTATCATGACAACAGCTTCCGCATCGCAAGCA
TCAAGCCCGTCATAAGTGAGACTCCCCTCCCCGTTGACCTCTACCGCTATGATGAGATTTCTGGCAAG
GTGGAACACTTTGGTAAGTTTGGAGTCATCTATTATGACATCAACCAGATCATCACCACTGCCGTGAT
GACCCTCAGCAAACACTTCGACACCCATGGGCGGATCAAGGAGGTCCAGTATGAGATGTTCCGGTCCC
TCATGTACTGGATGACGGTGCAATATGACAGCATGGGCAGGGTGATCAAGAGGGAGCTAAAACTGGGG
CCCTATGCCAATACCACGAAGTACACCTATGACTACGATGGGGACGGGCAGCTCCAGAGCGTGGCCGT
CAATGACCGCCCGACCTGGCGCTACAGCTATGACCTTAATGGGAATCTCCACTTACTGAACCCAGGCA
ACAGTGTGCGCCTCATGCCCTTGCGCTATGACCTCCGGGATCGGATAACCAGACTCGGGGATGTGCAG
TACAAAATTGACGACGATGGCTATCTGTGCCAGAGAGGGTCTGACATCTTCGAATACAATTCCAAGGG
CCTCCTAACAAGAGCCTACAACAAGGCCAGCGGGTGGAGTGTCCAGTACCGCTATGATGGCGTAGGAC
GGCGGGCTTCCTACAAGACCAACCTGGGCCACCACCTGCAGTACTTCTACTCTGACCTCCACAACCCG
ACGCGCATCACCCATGTCTACAATCACTCCAACTCGGAGATTACCTCACTGTACTACGACCTCCAGGG
CCACCTCTTTGCCATGGAGAGCAGCAGTGGGGAGGAGTACTATGTTGCCTCTGATAACACAGGGACTC
CTCTGGCTGTGTTCAGCATCAACGGCCTCATGATCAAACAGCTGCAGTACACGGCCTATGGGGAGATT
TATTATGACTCCAACCCCGACTTCCAGATGGTCATTGGCTTCCATGGGGGACTCTATGACCCCCTGAC
CAAGCTGGTCCACTTCACTCAGCGTGATTATGATGTGCTGGCAGGACGATGGACCTCCCCAGACTATA
CCATGTGGAAAAACGTGGGCAAGGAGCCGGCCCCCTTTAACCTGTATATGTTCAAGAGCAACAATCCT
CTCAGCAGTGAGCTAGATTTGAAGAACTACGTGACAGATGTGAAAAGCTGGCTTGTGATGTTTGGATT
TCAGCTTAGCAACATCATTCCTGGCTTCCCGAGAGCCAAAATGTATTTCGTGCCTCCTCCCTATGAAT
TGTCAGAGAGTCAAGCAAGTGAGAATGGACAGCTCATTACAGGTGTCCAACAGACAACAGAGAGACAT
AACCAGGCCTTCATGGCTCTGGAAGGACAGGTCATTACTAAAAAGCTCCACGCCAGCATCCGAGAGAA
AGCAGGTCACTGGTTTGCCACCACCACGCCCATCATTGGCAAAGGCATCATGTTTGCCATCAAAGAAG
GGCGGGTGACCACGGGCGTGTCCAGCATCGCCAGCGAAGATAGCCGCAAGGTGGCATCTGTGCTGAAC
AACGCCTACTACCTGGACAAGATGCACTACAGCATCGAGGGCAAGGACACCCACTACTTTGTGAAGAT
TGGCTCAGCCGATGGCGACCTGGTCACACTAGGCACCACCATCGGCCGCAAGGTGCTAGAGAGCGGGG
TGAACGTGACCGTGTCCCAGCCCACGCTGCTGGTCAACGGCAGGACTCGAAGGTTCACGAACATTGAG
TTCCAGTACTCCACGCTGCTGCTCAGCATCCGCTATGGCCTCACCCCCGACACCCTGGACGAAGAGAA
GGCCCGCGTCCTGGACCAGGCGAGACAGAGGGCCCTGGGCACGGCCTGGGCCAAGGAGCAGCAGAAAG
CCAGGGACGGGAGAGAGGGGAGCCGCCTGTGGACTGAGGGCGAGAAGCAGCAGCTTCTGAGCACCGGG
CGCGTGCAAGGGTACGAGGGATATTACGTGCTTCCCGTGGAGCAATACCCAGAGCTTGCAGACAGTAG
CAGCAACATCCAGTTTTTAAGACAGAATGAGATGGGAAAGAGGTAACAAAATAATCTGCTGCCATTCC
TTGTCTGAATGGCTCAGCAGGAGTAACTGTTATCTCCTCTCCTAAGGAGATGAAGACCTAACAGGGGC
ACTGCGGCTGGGCTGCTTTAGGAGACCAAGTGGCAAGAAAGCTCACATTTTTTGAGTTCAAATGCTAC
TGTCCAAGCGAGAAGTCCCTCATCCTGAAGTAGACTAAAGCCCGGCTGAAAATTCCGAGGAAAACAAA
ACAAACGAATGAATGAACAGACACACACAATGTTCCAAGTTCCCCTAAAATATGACCCACTTGTTCTG
GGTCTACGCAGAAAAGAGACGCAAAGTGTCCAAAAGGAACAAAAGAACAAAAACGAATAAGCAAAGAA
GAAAACAAACAAAAACAAAACAAAACAAACACACGGACCGATAAACAAAGAAGCGAAGATAAGAAAGA
AGGCCTCATATCCAATTACCTCACTCATTCACATGTGAGCGACACGCAGACATCCGCGAGGGCCAGCG
TCACCAGACCAGCTGCGGGACAAACCACTCAGACTGCTTGTAGGACAAATACTTCTGACATTTTCGTT
TAAGCAAATACAGGTGCATTTAAAACACGACTTTGGGGGTGATTTGTGTGTAGCGCCTGGGGAGGGGG
GATAAAAGAGGAGGAGTGAGCACTGGAAATACTTTTTAAAGAAAAAAAAACATGAGGGAATAAAAGAA
ATTCCTATCAAAAATCAAAGTGAAATAATACCATCCAGCACTTAACTCTCAGGTCCCAACTAAGTCTG
GCCTGAGCTAATTTATTTGAGCGCAGAGTGTAAAATTTAATTCAAAATGGTGGCTATAATCACTACAC
ATAAATTTCATACTCTTTTGTCTTTGGAGATTCCATTGTGGACAGTAATACGCAGTTACAGGGTGTAG
TCTGTTTAGATTCCGTAGTTCGTGGGTATCAGTTTCGGTAGAGGTGCAGCATCGTGACACTTTTGCTA
ACAGGTACCACTTCTGATCACCCTGTACATACATGAGCCGAAAGGCACAATCACTGTTTCAGATTTAA
AATTATTAGTGTGTTTGTTTGGTCCAGAAACTGAGACAATCACATGACAGTCACCACGAGGAGAGAAA
ATTTAAAAAATAAAAATAAAAACAAAAAAAATTTTAAAAATTAAAAAAACAAAAATAAAGTCTAATAA
GAACTTTGGTACAGGAACTTTTTTGTAATATACATGTATGAATTGTTCATCGAGTTTTTATATTAATT
TTAATTTGCTGCTAAGCAAAGACTAGGGACAGGCAAAGATAATTTATGGCAAAGTGTTTAAATTGTTT
ATACATAAATAAAGTCTCTAAAACTCCTGTG

| | | | |
|---|---|---|---|
| NOV7o, CG50426-08 | SEQ ID NO: 142 | 2733 aa | MW at 303423.3kD |
| Protein Sequence | | | |

MDVKDRRHRSLTRGRCGKECRYTSSSLDSEDCRVPTQKSYSSSETLKAYDHDSRMHYGNRVTDLIHRE
SDEFPRQGTNFTLAELGICEPSPHRSGYCSDMGILHQGYSLSTGSDADSDTEGGMSPEHAIRLWGRGI
KSRRSSGLSSRENSALTLTDSDNENKSDDENGRPIPPTSSPSLLPSAQLPSSHNPPPVSCQMPLLDSN
TSHQIMDTNPDEEFSPNSYLLRACSGPQQASSSGPPNHHSQSTLRPPLPPPHNHTLSHHHSSANSLNR
NSLTNRRSQIHAPAPAPNDLATTPESVQLQDSWVLNSNVPLETRHFLFKTSSGSTPLFSSSSPGYPLT
SGTVYTPPPRLLPRNTFSRKAFKLKKPSKYCSWKCAALSAIAAALLLAILLAYFIVPWSLKNSSIDSG
EAEVGRRVTQEVPPGVFWRSQIHISQPQFLKFNISLGKDALFGVYIRRGLPPSHAQYDFMERLDGKEK
WSVVESPRERRSIQTLVQNEAVFVQYLDVGLWHLAFYNDGKDKEMVSFNTVVLDSVQDCPRNCHGNGE
CVSGVCHCFPGFLGADCAKAACPVLCSGNGQYSKGTCQCYSGWKGAECDVPMNQCIDPSCGGHGSCID
GNCVCSAGYKGEHCEEVDCLDPTCSSHGVCVNGECLCSPGWGGLNCELARVQCPDQCSGHGTYLPDTG
LCSCDPNWMGPDCSVEVCSVDCGTHGVCIGGACRCEEGWTGAACDQRVCHPRCIEHGTCKDGKCECRE
GWNGEHCTIGRQTAGTETDGCPDLCNGNGRCTLGQNSWQCVCQTGWRGPGCNVAMETSCADNKDNEGD
GLVDCLDPDCCLQSACQNSLLCRGSRDPLDIIQQGQTDWPAVKSFYDRIKLLAGKDSTHIIPGENPFN
SSLVSLIRGQVVTTDGTPLVGVNVSFVKYPKYGYTITRQDGTFDLIANGGASLTLHFERAPFMSQERT
VWLPWNSFYAMDTLVMKTEENSIPSCDLSGFVRPDPIIISSPLSTFFSAAPGQNPIVPETQVLHEEIE
LPGSNVKLRYLSSRTAGYKSLLKITMTQSTVPLNLIRVHLMVAVEGHLFQKSFQASPNLASTFIWDKT
DAYGQRVYGLSDAVVSVGFEYETCPSLILWEKRTALLQGFELDPSNLGGWSLDKHHILNVKSGILHKG
TGENQFLTQQPAIITSIMGNGRRRSISCPSCNGLAEGNKLLAPVALAVGIDGSLYVGDFNYIRRIFPS
RNVTSILELRNKEFKHSNNPAHKYYLAVDPVSGSLYVSDTNSRRIYRVKSLSGTKDLAGNSEVVAGTG
EQCLPFDEARCGDGGKAIDATLMSPRGIAVDKNGLMYFVDATMIRKVDQNGIISTLLGSNDLTAVRPL
SCDSSMDVAQVRLEWPTDLAVNPMDNSLYVLENNVILRITENHQVSIIAGRPMHCQVPGIDYSLSKLA
IHSALESASAIAISHTGVLYITETDEKKINRLRQVTTNGEICLLAGAASDCDCKNDVNCNCYSGDDAY
ATDAILNSPSSLAVAPDGTIYIADLGNIRIRVSKNKPXTLNAFNQYEAASPGEQELYVFNADGIHQYT
VSLVTGEYLYNFTYSTDNDVTELIDNNGNSLKIRRDSSGMPRHLLMPDNQIITLTVGTNGGLKVVSTQ
NLELGLMTYDGNTGLLATKSDETGWTTFYDYDHEGRLTNVTRPTGVVTSLHREMEKSITIDIENSNRD
DDVTVITNLSSVEASYTVVQDQVRNSYQLCNNGTLRVMYANGMGISFHSEPHVLAGTITPTIGRCNIS
LPMENGLNSIEWRLRKEQIKGKVTIFGRKLRVHGRNLLSIDYDRNIRTEKIYDDHRKFTLRIIYDQVG
RPFLWLPSSGLAAVNVSYFFNGRIAGLQRGAMSERTDIDKQGRIVSRMFADGKVWSYSYLDKSMVLLL
QSQRQYIFEYDSSDRLLAVTMPSVARHSMSTHTSIGYIRNIYNPPESNASVIFDYSDDGRILKTSFLG
TGRQVFYKYGKLSKLSEIVYDSTAVTFGYDETTGVLKMVNLQSGGFSCTIRYRKIGPLVDKQIYRFSE
EGMVNARFDYTYHDNSFRIASIKPVISETPLPVDLYRYDEISGKVEHFGKFGVIYYDINQIITTAVMT
LSKHFDTHGRIKEVQYEMFRSLMYWMTVQYDSMGRVIKRELKLGPYANTTKYTYDYDGDGQLQSVAVN
DRPTWRYSYDLNGNLHLLNPGNSVRLMPLRYDLRDRITRLGDVQYKIDDDGYLCQRGSDIFEYNSKGL
LTRAYNKASGWSVQYRYDGVGRRASYKTNLGHHLQYFYSDLHNPTRITHVYNHSNSEITSLYYDLQGH
LFAMESSSGEEYYVASDNTGTPLAVFSINGLMIKQLQYTAYGEIYYDSNPDFQMVIGFHGGLYDPLTK
LVHFTQRDYDVLAGRWTSPDYTMWKNVGKEPAPFNLYMFKSNNPLSSELDLKNYVTDVKSWLVMFGFQ
LSNIIPGFPRAKMYFVPPPYELSESQASENGQLITGVQQTTERHNQAFMALEGQVITKKLHASIREKA
GHWFATTTPIIGKGIMFAIKEGRVTTGVSSIASEDSRKVASVLNNAYYLDKMHYSIEGKDTHYFVKIG
SADGDLVTLGTTIGRKVLESGVNVTVSQPTLLVNGRTRRFTNIEFQYSTLLLSIRYGLTPDTLDEEKA
RVLDQARQRALGTAWAKEQQKARDGREGSRLWTEGEKQQLLSTGRVQGYEGYYVLPVEQYPELADSSS
NIQFLRQNEMGKR

| | | |
|---|---|---|
| Nov7p, CG50426-09 | SEQ ID NO: 143 | 1920 bp |
| DNA Sequence | ORF Start: at 180 | ORF Stop: at 1920 |

CCAGCATTAGATGAGTTGACAAAAATGCAGTTTCAGCTCTGAAGGTCTGAAAGATTCTGCTGCAACTA
AAGCTCTGAAGATTCTGCTACAACTATGACATCCATTTTCTCCCACTTCAGACAGGATGAATACAAGG
TGGCAAAGTGACAAGTGCCAAAACTCAGGCCTGACTTTCCTGAAAACATCAGCATTCTGCCATATCTG
GAATAATGGATGTAAAGGACCGGCGACACCGCTCTTTGACCAGAGGACGCTGTGGCAAAGAGTGTCGC
TACACAAGCTCCTCTCTGGACAGTGAGGACTGCCGGGTGCCCACACAGAAATCCTACAGCTCCAGTGA
GACTCTGAAGGCCTATGACCATGACAGCAGGATGCACTATGGAAACCGAGTCACAGACCTCATCCACC
GGGAGTCAGATGAGTTTCCTAGACAAGGAACCAACTTCACCCTTGCCGAACTGGGCATCTGTGAGCCC
TCCCCACACCGAAGCGGCTACTGCTCCGACATGGGGATCCTTCACCAGGGCTACTCCCTTAGCACAGG
GTCTGACGCCGACTCCGACACCGAGGGAGGGATGTCTCCAGAACACGCCATCAGACTGTGGGGCAGAG
GGATAAAATCCAGGCGCAGTTCCGGCCTGTCCAGTCGTGAAAACTCGGCCCTTACCCTGACTGACTCT
GACAACGAAAACAAATCAGATGATGAGAACGGTCGTCCCATTCCACCTACATCCTCGCCTAGTCTCCT
CCCATCTGCTCAGCTGCCTAGCTCCCATAATCCTCCACCAGTTAGCTGCCAGATGCCATTGCTAGACA
GCAACACCTCCCATCAAATCATGGACACCAACCCTGATGAGGAATTCTCCCCCAATTCATACCTGCTC
AGAGCATGCTCAGGGCCCCAGCAAGCCTCCAGCAGTGGCCCTCCGAACCACCACAGCCAGTCGACTCT
GAGGCCCCCTCTCCCACCCCCTCACAACCACACGCTGTCCCATCACCACTCGTCCGCCAACTCCCTCA
ACAGGAACTCACTGACCAATCGGCGGAGTCAGATCCACGCCCCGGCCCCAGCGCCCAATGACCTGGCC
ACCACACCAGAGTCCGTTCAGCTTCAGGACAGCTGGGTGCTAAACAGCAACGTGCCACTGGAGACCCG
GCACTTCCTCTTCAAGACCTCCTCGGGGAGCACACCCTTGTTCAGCAGCTCTTCCCCGGGATACCCTT
TGACCTCAGGAACGGTTTACACGCCCCCGCCCCGCCTGCTGCCCAGGAATACTTTCTCCAGGAAGGCT
TTCAAGCTGAAGAAGCCCTCCAAATACTGCAGCTGGAAATGTGCTGCCCTCTCCGCCATTGCCGCGGC
CCTCCTCTTGGCTATTTTGCTGGCGTATTTCATAGTGCCCTGGTCGTTGAAAAACAGCAGCATAGACA
GTGGTGAAGCAGAAGTTGGTCGGCGGGTAACACAAGAAGTCCCACCAGGGGTGTTTTGGAGGTCACAA
ATTCACATCAGTCAGCCCCAGTTCTTAAAGTTCAACATCTCCCTCGGGAAGGACGCTCTCTTTGGTGT
TTACATAAGAAGAGGACTTCCACCATCTCATGCCCAGTATGACTTCATGGAACGTCTGGACGGGAAGG
AGAAGTGGAGTGTGGTTGAGTCTCCCAGGGAACGCCGGAGCATACAGACCTTGGTTCAGAATGAAGCC
GTGTTTGTGCAGTACCTGGATGTGGGCCTGTGGCATCTGGCCTTCTACAATGATGGAAAAGACAAAGA
GATGGTTTCCTTCAATACTGTTGTCCTAGATTCAGTGCAGGACTGTCCACGTAACTGCCATGGGAATG
GTGAATGTGTGTCCGGGGTGTGTCACTGTTTCCCAGGATTTCTAGGAGCAGACTGTGCTAAAGCTGCC
TGCCCTGTCCTGTGCA

| | | | |
|---|---|---|---|
| NOV7p, CG50426-09 | SEQ ID NO: 144 | 580 aa | MW at 63961.7kD |
| Protein Sequence | | | |

KHQHSAISGIMDVKDRRHRSLTRGRCGKECRYTSSSLDSEDCRVPTQKSYSSSETLKAYDHDSPNHYG
NRVTDLIHRESDEFPRQGTNFTLAELGICEPSPHRSGYCSDMGILHQGYSLSTGSDADSDTEGGMSPE
HAIRLWGRGIKSRRSSGLSSRENSALTLTDSDNENKSDDENGRPIPPTSSPSLLPSAQLPSSHNPPPV
SCQMPLLDSNTSHQIMDTNPDEEFSPNSYLLRACSGPQQASSSGPPNHHSQSTLRPPLPPPHNHTLSH
HHSSANSLNRNSLTNRRSQIHAPAPAPNDLATTPESVQLQDSWVLNSNVPLETRHFLFKTSSGSTPLF
SSSSPGYPLTSGTVYTPPPRLLPRNTFSRKAFKLKKPSKYCSWKCAALSAIAAALLLAILLAYFIVPW
SLKNSSIDSGEAEVGRRVTQEVPPGVFWRSQIHTSQPQFLKFNISLGKDALFGVYIRRGLPPSHAQYD
FMERLDGKEKWSVVESPRERRSIQTLVQNEAVFVQYLDVGLWHLAFYNDGKDKEMVSFNTVVLDSVQD
CPRNCHGNGECVSGVCHCFPGFLGADCAKAACPVLC

| | | |
|---|---|---|
| NOV7q, CG50426-10 | SEQ ID NO: 145 | 9756 bp |
| DNA Sequence | ORF Start: ATG at 210 | ORF Stop: TAA at 8409 |

CCAGCATTAGATGAGTTGACAAAAATGCAGTTTCAGCTCTGAAGGTCTGAAAGATTCTGCTGCAACTA
AAGCTCTGAAGATTCTGCTACAACTATGACATCCATTTTCTCCCACTTCAGACAGGATGAATACAAGG
TGGCAAAGTGACAAGTGCCAAAACTCAGGCCTGACTTTTCTGAAAACATCAGCATTCTGCCATATCTG
GAATAATGGATGTAAAGGACCGGCGACACCGCTCTTTGACCAGAGGACGCTGTGGCAAAGAGTGTCGC
TACACAAGCTCCTCTCTGGACAGTGAGGACTGCCGGGTGCCCACACAGAAATCCTACAGCTCCAGTGA
GACTCTGAAGGCCTATGACCATGACAGCAGGATGCACTATGGAAACCGAGTCACAGACCTCATCCACC
GGGAGTCAGATGAGTTTCCTAGACAAGGAACCAACTTCACCCTTGCCGAACTGGGCATCTGTGAGCCC
TCCCCACACCGAAGCGGCTACTGCTCCGACATGGGGATCCTTCACCAGGGCTACTCCCTTAGCACAGG
GTCTGACGCCGACTCCGACACCGAGGGAGGGATGTCTCCAGAACACGCCATCAGACTGTGGGGCAGAG
GGATAAAATCCAGGCGCAGTTCCGGCCTGTCCAGTCGTGAAAACTCGGCCCTTACCCTGACTGACTCT
GACAACGAAAACAAATCAGATGATGAGAACGGTCGTCCCATTCCACCTACATCCTCGCCTAGTCTCCT
CCCATCTGCTCAGCTGCCTAGCTCCCATAATCCTCCACCAGTTAGCTGCCAGATGCCATTGCTAGACA
GCAACACCTCCCATCAAATCATGGACACCAACCCTGATGAGGAATTCTCCCCCAATTCATACCTGCTC
AGAGCATGCTCAGGGCCCCAGCAAGCCTCCAGCAGTGGCCCTCCGAACCACCACAGCCAGTCGACTCT
GAGGCCCCCTCTCCCACCCCCTCACAACCACACGCTGTCCCATCACCACTCGTCCGCCAACTCCCTCA
ACAGGAACTCACTGACCAATCGGCGGAGTCAGATCCACGCCCCGGCCCCAGCGCCCAATGACCTGGCC
ACCACACCAGAGTCCGTTCAGCTTCAGGACAGCTGGGTGCTAAACAGCAACGTGCCACTGGAGACCCG
GCACTTCCTCTTCAAGACCTCCTCGGGGAGCACACCCTTGTTCAGCAGCTCTTCCCCGGGATACCCTT
TGACCTCAGGAACGGTTTACACGCCCCCGCCCCGCCTGCTGCCCAGGAATACTTTCTCCAGGAAGGCT
TTCAAGCTGAAGAAGCCCTCCAAATACTGCAGCTGGAAATGTGCTGCCCTCTCCGCCATTGCCGCGGC
CCTCCTCTTGGCTATTTTGCTGGCGTATTTCATAGTGCCCTGGTCGTTGAAAAACAGCAGCATAGACA
GTGGTGAAGCAGAAGTTGGTCGGCGGGTAACACAAGAAGTCCCACCAGGGGTGTTTTGGAGGTCACAA
ATTCACATCAGTCAGCCCCAGTTCTTAAAGTTCAACATCTCCCTCGGGAAGGACGCTCTCTTTGGTGT
TTACATAAGAAGAGGACTTCCACCATCTCATGCCCAGTATGACTTCATGGAACGTCTGGACGGGAAGG
AGAAGTGGAGTGTGGTTGAGTCTCCCAGGGAACGCCGGAGCATACAGACCTTGGTTCAGAATGAAGCC
GTGTTTGTGCAGTACCTGGATGTGGGCCTGTGGCATCTGGCCTTCTACAATGATGGAAAAGACAAAGA
GATGGTTTCCTTCAATACTGTTGTCCTAGATTCAGTGCAGGACTGTCCACGTAACTGCCATGGGAATG
GTGAATGTGTGTCCGGGGTGTGTCACTGTTTCCCAGGATTTCTAGGAGCAGACTGTGCTAAAGCTGCC
TGCCCTGTCCTGTGCAGTGGGAATGGACAATATTCTAAAGGGACGTGCCAGTGCTACAGCGGCTGGAA
AGGTGCAGAGTGCGACGTGCCCATGAATCAGTGCATCGATCCTTCCTGCGGGGGCCACGGCTCCTGCA
TTGATGGGAACTGTGTCTGCTCTGCTGGCTACAAAGGCGAGCACTGTGAGGAAGTTGATTGCTTGGAT
CCCACCTGCTCCAGCCACGGAGTCTGTGTGAATGGAGAATGCCTGTGCAGCCCTGGCTGGGGTGGTCT
GAACTGTGAGCTGGCGAGGGTCCAGTGCCCAGACCAGTGCAGTGGGCATGGCACGTACCTGCCTGACA
CGGGCCTCTGCAGCTGCGATCCCAACTGGATGGGTCCCGACTGCTCTGTTGAAGTGTGCTCAGTAGAC
TGTGGCACTCACGGCGTCTGCATCGGGGGAGCCTGCCGCTGTGAAGAGGGCTGGACAGGCGCAGCGTG
TGACCAGCGCGTGTGCCACCCCCGCTGCATTGAGCACGGGACCTGTAAAGATGGCAAATGTGAATGCC
GAGAGGGCTGGAATGGTGAACACTGCACCATTGGTAGGCAAACGGCAGGCACCGAAACAGATGGCTGC
CCTGACTTGTGCAACGGTAACGGGAGATGCACACTGGGTCAGAACAGCTGGCAGTGTGTCTGCCAGAC
CGGCTGGAGAGGGCCCGGATGCAACGTTGCCATGGAAACTTCCTGTGCTGATAACAAGGATAATGAGG
GAGATGGCCTGGTGGATTGTTTGGACCCTGACTGCTGCCTGCAGTCAGCCTGTCAGAACAGCCTGCTC
TGCCGGGGGTCCCGGGACCCACTGGACATCATTCAGCAGGGCCAGACGGATTGGCCCGCAGTGAAGTC
CTTCTATGACCGTATCAAGCTCTTGGCAGGCAAGGATAGCACCCACATCATTCCTGGAGAGAACCCTT
TCAACAGCAGCTTGGTTTCTCTCATCCGAGGCCAAGTAGTAACTACAGATGGAACTCCCCTGGTCAAT
GTGAACGTGTCTTTTGTCAAGTACCCAAAATACGGCTACACCATCACCCGCCAGGATGGCACGTTCGA
CCTGATCGCAAATGGAGGTGCTTCCTTGACTCTACACTTTGAGCGAGCCCCGTTCATGAGCCAGGAGC
GCACTGTGTGGCTGCCGTGGAACAGCTTTTACGCCATGGACACCCTGGTGATGAAGACCGAGGAGAAC
TCCATCCCCAGCTGTGACCTCAGTGGCTTTGTCCGGCCTGATCCAATCATCATCTCCTCCCCACTGTC
CACCTTCTTTAGTGCTGCCCCTGGGCAGAATCCCATCGTGCCTGAGACCCAGGTTCTTCATGAAGAAA
TCGAGCTCCCTGGTTCCAATGTGAAACTTCGCTATCTGAGCTCTAGAACTGCAGGGTACAAGTCACTG
CTGAAGATCACCATGACCCAGTCCACAGTGCCCCTGAACCTCATTAGGGTTCACCTGATGGTGGCTGT
CGAGGGGCATCTCTTCCAGAAGTCATTCCAGGCTTCTCCCAACCTGGCCTCCACCTTCATCTGGGACA
AGACAGATGCGTATGGCCAAAGGGTGTATGGACTCTCAGATGCTGTTGTGTCTGTCGGGTTTGAATAT
GAGACCTGTCCCAGTCTAATTCTCTGGGAGAAAAGGACAGCCCTCCTTCAGGGATTCGAGCTGGACCC
CTCCAACCTCGGTGGCTGGTCCCTAGACAACACCACATCCTCAATGTTAAAAGTGGAAATCCTACACA
AAGGCACTGGGGAAAACCAGTTCCTGACCCAGCAGCCTGCCATCATCACCAGCATCATGGGCAATGGT
CGCCGCCGGAGCATTTCCTGTCCCAGCTGCAACGGCCTTGCTGAAGGCAACAAGCTGCTGGCCCCAGT
GGCTCTGGCTGTTGGAATCGATGGGAGCCTCTATGTGGGTGACTTCAATTACATCCGACGCATCTTTC
CCTCTCGAAATGTGACCAGCATCTTGGAGTTACGAAATAAAGAGTTTAAACATAGCAACAACCCAGCA
CACAAGTACTACTTGGCAGTGGACCCCGTGTCCGGCTCGCTCTACGTGTCCGACACCAACAGCAGGAG
AATCTACCGCGTCAAGTCTCTGAGTGGAACCAAAGACCTGGCTGGGAATTCGGAAGTTGTGGCAGGGA
CGGGAGAGCAGTGTCTACCCTTTGATGAAGCCCGCTGCGGGGATGGAGGGAAGGCCATAGATGCAACC
CTGATGAGCCCGAGAGGTATTGCAGTAGACAAGAATGGGCTCATGTACTTTGTCGATGCCACCATGAT
CCGGAAGGTTGACCAGAATGGAATCATCTCCACCCTGCTGGGCTCCAATGACCTCACTGCCGTCCGGC
CGCTGAGCTGTGATTCCAGCATGGATGTAGCCCAGGTTCGTCTGGAGTGGCCAACAGACCTTGCTGTC
AATCCCATGGATAACTCCTTGTATGTTCTAGAGAACAATGTCATCCTTCGAATCACCGAGAACCACCA
AGTCAGCATCATTGCGGGACGCCCCATGCACTGCCAAGTTCCTGGCATTGACTACTCACTCAGCAAAC
TAGCCATTCACTCTGCCCTGGAGTCAGCCAGTGCCATTGCCATTTCTCACACTGGGGTCCTCTACATC
ACTGAGACAGATGAGAAGAAGATTAACCGTCTACGCCAGGTAACAACCAACGGGGAGATCTGCCTTTT
AGCTGGGGCAGCCTCGGACTGCGACTGCAAAAACGATGTCAATTGCAACTAATATTCAGGAGATGATG
CCTACGCGACTGATGCCATCTTGAATTCCCCATCATCCTTAGCTGTAGCTCCAGATGGTACCATTTAC
ATTGCAGACCTTGGAAATATTCGGATCAGGGCGGTCAGCAAGAACAAGCCTGTTCTTAATGCCTTCAA
CCAGTATGAGGCTGCATCCCCCGGAGAGCAGGAGTTATATGTTTTCAACGCTGATGGCATCCACCAAT
ACACTGTGAGCCTGGTGACAGGGGAGTACTTGTACAATTTCACATATAGTACTGACAATGATGTCACT
GAATTGATTGACAATAATGGGAATTCCCTGAAGATCCGTCGGGACAGCAGTGGCATGCCCCGTCACCT
GCTCATGCCTGACAACCAGATCATCACCCTCACCGTGGGCACCAATGGAGGCCTCAAAGTCGTGTCCA
CACAGAACCTGGAGCTTGGTCTCATGACCTATGATGGCAACACTGGGCTCCTGGCCACCAAGAGCGAT
GAAACAGGATGGACGACTTTCTATGACTATGACCACGAAGGCCGCCTGACCAACGTGACAAGCCCCAC
GGGGGTGGTAACCAGTCTGCACCGGGAAATGGAGAAATCTATTACCATTGACATTGAGAACTCCAACC
GTGATGATGACGTCACTGTCATCACCAACCTCTCTTCAGTAGAGGCCTCCTACACAGTGGTACAAGAT
CAAGTTCGGAACAGCTACCAGCTCTGTAATAATGGTACCCTGAGGGTGATGTATGCTAATGGGATGGG
TATCAGCTTCCACAGCGAGCCCCATGTCCTAGCGGGCACCATCACCCCCACCATTGGACGCTGCAACA
TCTCCCTGCCTATGGAGAATGGCTTAAACTCCATTGAGTGGCGCCTAAGAAAGGAACAGATTAAAGGC
AAAGTCACCATCTTTGGCAGGAAGCTCCGGGTCCATGGAAGAAATCTCTTGTCCATTGACTATGATCG
AAATATTCGGACTGAAAAGATCTATGATGACCACCGGAAGTTCACCCTGAGGATCATTTATGACCAGG
TGGGCCGCCCCTTCCTCTGGCTGCCCAGCAGCGGGCTGGCAGCTGTCAACGTGTCATACTTCTTCAAT
GGGCGCCTGGCTGGGCTTCAGCGTGGGGCCATGAGCGAGAGGACAGACATCGACAAGCAAGGCCGCAT
CGTGTCCCGCATGTTCGCTGACGGGAAAGTGTGGAGCTACTCCTACCTTGACAAGTCCATGGTCCTCC
TGCTTCAGAGCCAACGTCAGTATATATTTGAGTATGACTCCTCTGACCGCCTCCTTGCCGTCACCATG
CCCAGCGTGGCCCGGCACAGCATGTCCACACACACCTCCATCGGCTACATCCGTAATATTTACAACCC
GCCTGAAAGCAATGCTTCGGTCATCTTTGACTACAGTGATGACGGCCGCATCCTGAAGACCTCCTTTT
TGGGCACCGGACGCCAGGTGTTCTACAAGTATGGGAAACTCTCCAAGTTATCAGAGATTGTCTACGAC
AGTACCGCCGTCACCTTCGGGTATGACGAGACCACTGGTGTCTTGAAGATGGTCAACCTCCAAAGTGG
GGGCTTCTCCTGCACCATCAGGTACCGGAAGATTGGCCCCCTGGTGGACAAGCAGATCTACAGGTTCT
CCGAGGAAGGCATGGTCAATGCCAGGTTTGACTACACCTATCATGACAACAGCTTCCGCATCGCAAGC
ATCAAGCCCGTCATAAGTGAGACTCCCCTCCCCGTTGACCTCTACCGCTATGATGAGATTTCTGGCAA
GGTGGAACACTTTGGTAAGTTTGGAGTCATCTATTATGACATCAACCAGATCATCACCACTGCCGTGA
TGACCCTCAGCAAACACTTCGACACCCATGGGCGGATCAAGGAGGTCCAGTATGAGATGTTCCGGTCC
CTCATGTACTGGATGACGGTGCAATATGACAGCATGGGCAGGGTGATCAAGAGGGAGCTAAAACTGGG
GCCCTATGCCAATACCACGAAGTACACCTATGACTACGATGGGGACGGGCAGCTCCAGAGCGTGGCCG
TCAATGACCGCCCGACCTGGCGCTACAGCTATGACCTTAATGGGAATCTCCACTTACTGAACCCAGGC
AACAGTGTGCGCCTCATGCCCTTGCGCTATGACCTCCGGGATCGGATAACCAGACTCGGGGATGTGCA
GTACAAAATTGACGACGATGGCTATCTGTGCCAGAGAGGGTCTGACATCTTCGAATACAATTCCAAGG
GCCTCCTAACAAGAGCCTACAACAAGGCCAGCGGGTGGAGTGTCCAGTACCGCTATGATGGCGTAGGA
CGGCGGGCTTCCTACAAGACCAACCTGGGCCACCACCTGCAGTACTTCTACTCTGACCTCCACAACCC
GACGCGCATCACCCATGTCTACAATCACTCCAACTCGGAGATTACCTCACTGTACTACGACCTCCAGG
GCCACCTCTTTGCCATGGAGAGCAGCAGTGGGGAGGAGTACTATGTTGCCTCTGATAACACAGGGACT
CCTCTGGCTGTGTTCAGCATCAACGGCCTCATGATCAAACAGCTGCAGTACACGGCCTATGGGGAGAT
TTATTATGACTCCAACCCCGACTTCCAGATGGTCATTGGCTTCCATGGGGGACTCTATGACCCCCTGA
CCAAGCTGGTCCACTTCACTCAGCGTGATTATGATGTGCTGGCAGGACGATGGACCTCCCCAGACTAT
ACCATGTGGAAAAACGTGGGCAAGGAGCCGGCCCCCTTTAACCTGTATATGTTCAAGAGCAACAATCC
TCTCAGCAGTGAGCTAGATTTGAAGAACTACGTGACAGATGTGAAAAGCTGGCTTGTGATGTTTGGAT
TTCAGCTTAGCAACATCATTCCTGGCTTCCCGAGAGCCAAAATGTATTTCGTGCCTCCTCCCTATGAA
TTGTCAGAGAGTCAAGCAAGTGAGAATGGACAGCTCATTACAGGTGTCCAACAGACAACAGAGAGACA
TAACCAGGCCTTCATGGCTCTGGAAGGACAGGTCATTACTAAAAAGCTCCACGCCAGCATCCGAGAGA
AAGCAGGTCACTGGTTTGCCACCACCACGCCCATCATTGGCAAAGGCATCATGTTTGCCATCAAAGAA
GGGCGGGTGACCACGGGCGTGTCCAGCATCGCCAGCGAAGATAGCCGCAAGGTGGCATCTGTGCTGAA
CAACGCCTACTACCTGGACAAGATGCACTACAGCATCGAGGGCAAGGACACCCACTACTTTGTGAAGA
TTGGCTCAGCCGATGGCGACCTGGTCACACTAGGCACCACCATCGGCCGCAAGGTGCTAGAGAGCGGG
GTGAACGTGACCGTGTCCCAGCCCACGCTGCTGGTCAACGGCAGGACTCGAAGGTTCACGAACATTGA
GTTCCAGTACTCCACGCTGCTGCTCAGCATCCGCTATGGCCTCACCCCCGACACCCTGGACGAAGAGA
AGGCCCGCGTCCTGGACCAGGCGAGACAGAGGGCCCTGGGCACGGCCTGGGCCAAGGAGCAGCAGAAA
GCCAGGGACGGGAGAGAGGGGAGCCGCCTGTGGACTGAGGGCGAGAAGCAGCAGCTTCTGAGCACCGG
GCGCGTGCAAGGGTACGAGGGATATTACGTGCTTCCCGTGGAGCAATACCCAGAGCTTGCAGACAGTA
GCAGCAACATCCAGTTTTTAAGACAGAATGAGATGGGAAAGAGGTAACAAAATAATCTGCTGCCATTC
CTTGTCTGAATGGCTCAGCAGGAGTAACTGTTATCTCCTCTCCTAAGGAGATGAAGACCTAACAGGGG
CACTGCGGCTGGGCTGCTTTAGGAGACCAAGTGGCAAGAAAGCTCACATTTTTTGAGTTCAAATGCTA
CTGTCCAAGCGAGAAGTCCCTCATCCTGAAGTAGACTAAAGCCCGGCTGAAAATTCCGAGGAAAACAA
AACAAACGAATGAATGAACAGACACACACAATGTTCCAAGTTCCCCTAAAATATGACCCACTTGTTCT
GGGTCTACGCAGAAAAGAGACGCAAAGTGTCCAAAAGGAACAAAAGAACAAAAACGAATAAGCAAAGA
AGAAAACAAACAAAAACAAAACAAAACAAACACACGGACCGATAAACAAAGAAGCGAAGATAAGAAAG
AAGGCCTCATATCCAATTACCTCACTCATTCACATGTGAGCGACACGCAGACATCCGCGAGGGCCAGC
GTCACCAGACCAGCTGCGGGACAAACCACTCAGACTGCTTGTAGGACAAATACTTCTGACATTTTCGT
TTAAGCAAATACAGGTGCATTTAAAACACGACTTTGGGGGTGATTTGTGTGTAGCGCCTGGGGAGGGG
GGATAAAAGAGGAGGAGTGAGCACTGGAAATACTTTTTAAAGAAAAAAAAACATGAGGGAATAAAAGA
AATTCCTATCAAAAATCAAAGTGAAATAATACCATCCAGCACTTAACTCTCAGGTCCCAACTAAGTCT
GGCCTGAGCTAATTTATTTGAGCGCAGAGTGTAAAATTTAATTCAAAATGGTGGCTATAATCACTACA
GATAAATTTCATACTCTTTTGTCTTTGGAGATTCCATTGTGGACAGTAATACGCAGTTACAGGGTGTA
GTCTGTTTAGATTCCGTAGTTCGTGGGTATCAGTTTCGGTAGAGGTGCAGCATCGTGACACTTTTGCT
AACAGGTACCACTTCTGATCACCCTGTACATACATGAGCCGAAAGGCACAATCACTGTTTCAGATTTA
AAATTATTAGTGTGTTTGTTTGGTCCAGAAACTGAGACAATCACATGACAGTCACCACGAGGAGAGAA
AATTTAAAAAATAAAAATAAAAACAAAAAAAATTTTAAAAATTAAAAAAACAAAAATAAAGTCTAATA
AGAACTTTGGTACAGGAACTTTTTTGTAATATACATGTATGAATTGTTCATCGAGTTTTTATATTAAT
TTTAATTTGCTGCTAAGCAAAGACTAGGGACAGGCAAAGATAATTTATGGCAAAGTGTTTAAATTGTT
TATACATAAATAAAGTCTCTAAAACTCCTGTG

| | | | |
|---|---|---|---|
| NOV7q, CG50426-10 | SEQ ID NO: 146 | 2733 aa | MW at 303423.3kD |
| Protein Sequence | | | |

MDVKDRRHRSLTRGRCGKECRYTSSSLDSEDCRVPTQKSYSSSETLKAYDHDSRMHYGNRVTDLIHRE
SDEFPRQGTNFTLAELGICEPSPHRSGYCSDMGILHQGYSLSTGSDADSDTEGGMSPEHAIRLWGRGI
KSRRSSGLSSRENSALTLTDSDNENKSDDENGRPIPPTSSPSLLPSAQLPSSHNPPPVSCQMPLLDSN
TSHQIMDTNPDEEFSPNSYLLRACSGPQQASSSGPPNHHSQSTLRPPLPPPHNHTLSHHHSSANSLNR
NSLTNRRSQIHAPAPAPNDLATTPESVQLQDSWVLNSNVPLETRHFLFKTSSGSTPLFSSSSPGYPLT
SGTVYTPPPRLLPRNTFSRKAFKLKKPSKYCSWKCAALSAIAAALLLAILLAYFIVPWSLKNSSIDSG
EAEVGRRVTQEVPPGVFWRSQIHISQPQFLKFNISLGKDALFGVYTRRGLPPSHAQYDFMERLDGKEK
WSVVESPRERRSIQTLVQNEAVFVQYLDVGLWHLAFYNDGKDKEMVSFNTVVLDSVQDCPRNCNGNGE
CVSGVCHCFPGFLGADCAKAACPVLCSGNGQYSKGTCQCYSGWKGAECDVPMNQCIDPSCGGHGSCID
GNCVCSAGYKGEHCEEVDCLDPTCSSHGVCVNGECLCSPGWGGLNCELARVQCPDQCSGHGTYLPDTG
LCSCDPNWMGPDCSVEVCSVDCGTHGVCIGGACRCEEGWTGAACDQRVCHPRCIENGTCKDGKCECRE
GWNGEHCTIGRQTAGTETDGCPDLCNGNGRCTLGQNSWQCVCQTGWRGPGCNVAMETSCADNKDNEGD
GLVDCLDPDCCLQSACQNSLLCRGSRDPLDIIQQGQTDWPAVKSFYDRIKLLAGKDSTHIIPGENPFN
SSLVSLIRGQVVTTDGTPLVGVNVSFVKYPKYGYTITRQDGTFDLTANGGASLTLHFERAPFMSQERT
VWLPWNSFYAMDTLVMKTEENSIPSCDLSGFVRPDPIIISSPLSTFFSAAPGQNPIVPETQVLHEEIE
LPGSNVKLRYLSSRTAGYKSLLKITMTQSTVPLNLIRVHLMVAVEGHLFQKSFQASPNLASTFIWDKT
DAYGQRVYGLSDAVVSVGFEYETCPSLILWEKRTALLQGFELDPSNLGGWSLDKHHILNVKSGILHKG
TGENQFLTQQPAIITSTMGNGRRRSISCPSCNGLAEGNKLLAPVALAVGIDGSLYVGDFNYIRRIFPS
RNVTSILELRNKEFKHSNNPAHKYYLAVDPVSGSLYVSDTNSRRIYRVKSLSGTKDLAGNSEVVAGTG
EQCLPFDEARCGDGGKAIDATLMSPRGIAVDKNGLMYFVDATMIRKVDQNGIISTLLGSNDLTAVRPL
SCDSSMDVAQVRLEWPTDLAVNPMDNSLYVLENNVILRITENHQVSIIAGRPMHCQVPGIDYSLSKLA
IHSALESASAIAISHTGVLYITETDEKKINRLRQVTTNGEICLLAGAASDCDCKNDVNCNCYSGDDAY
ATDAILNSPSSLAVAPDGTIYIADLGNIRIRAVSKNKPVLNAFNQYEAASPGEQELYVFNADGIHQYT
VSLVTGEYLYNFTYSTDNDVTELIDNNGNSLKIRRDSSGMPRHLLMPDNQITTLTVGTNGGLKVVSTQ
NLELGLMTYDGNTGLLATKSDETGWTTFYDYDHEGRLTNVTRPTGVVTSLHREMEKSITIDIENSNRD
DDVTVITNLSSVEASYTVVQDQVRNSYQLCNNGTLRVMYANGMGISFHSEPHVLAGTITPTIGRCNIS
LPMENGLNSIEWRLRKEQIKGKVTIFGRKLRVHGRNLLSIDYDRNIRTEKIYDDHRKFTLRIIYDQVG
RPFLWLPSSGLAAVNVSYFFNGRLAGLQRGAMSERTDIDKQGRIVSRMFADGKVWSYSYLDKSMVLLL
QSQRQYIFEYDSSDRLLAVTMPSVARHSMSTHTSIGYIRNIYNPPESNASVIFDYSDDGRILKTSFLG
TGRQVFYKYGKLSKLSEIVYDSTAVTFGYDETTGVLKMVNLQSGGFSCTIRYPYIGPLVDKQIYRFSE
EGMVNARFDYTYHDNSFRIASIKPVISETPLPVDLYRYDEISGKVEHFGKFGVIYYDINQIITTAVMT
LSKHFDTHGRIKEVQYEMFRSLMYWMTVQYDSMGRVIKRELKLGPYANTTKYTYDYDGDGQLQSVAVN
DRPTWRYSYDLNGNLHLLNPGNSVRLMPLRYDLRDRITRLGDVQYKIDDDGYLCQRGSDIFEYNSKGL
LTRAYNKASGWSVQYRYDGVGRRASYKTNLGHHLQYFYSDLHNPTRITHVYNHSNSEITSLYYDLQGH
LFAMESSSGEEYYVASDNTGTPLAVFSINGLMIKQLQYTAYGEIYYDSNPDFQMVTGFHGGLYDPLTK
LVHFTQRDYDVLAGRWTSPDYTMWKNVGKEPAPFNLYMFKSNNPLSSELDLKNYVTDVKSWLVMFGFQ
LSNIIPGFPRAKMYFVPPPYELSESQASENGQLITGVQQTTERHNQAFMALEGQVITKKLHASIREKA
GHWFATTTPIIGKGIMFATKEGRVTTGVSSIASEDSRKVASVLNNAYYLDKMHYSIEGKDTHYFVKIG
SADGDLVTLGTTIGRKVLESGVNVTVSQPTLLVNGRTRRFTNIEFQYSTLLLSIRYGLTPDTLDEEKA
RVLDQARQRALGTAWAKEQQKARDGREGSRLWTEGEKQQLLSTGRVQGYEGYYVLPVEQYPELADSSS
NIQFLRQNEMGKR

| | | |
|---|---|---|
| NOV7r, CG50426-11 | SEQ ID NO: 147 | 9729 bp |
| DNA Sequence | ORF Start: ATG at 210 | ORF Stop: TAA at 8382 |

CCAGCATTAGATGAGTTGACAAAAATGCAGTTTCAGCTCTGAAGGTCTGAAAGATTCTGCTGCAACTA
AAGCTCTGAAGATTCTGCTACAACTATGACATCCATTTTCTCCCACTTCAGACAGGATGAATACAAGG
TGGCAAAGTGACAAGTGCCAAAACTCAGGCCTGACTTTCCTGAAAACATCAGCATTCTGCCATATCTG
GAATAATGGATGTAAAGGACCGGCGACACCGCTCTTTGACCAGAGGACGCTGTGGCAAAGAGTGTCGC
TACACAAGCTCCTCTCTGGACAGTGAGGACTGCCGGGTGCCCACACAGAAATCCTACAGCTCCAGTGA
GACTCTGAAGGCCTATGACCATGACAGCAGGATGCACTATGGAAACCGAGTCACAGACCTCATCCACC
GGGAGTCAGATGAGTTTCCTAGACAAGGAACCAACTTCACCCTTGCCGAACTGGGCATCTGTGAGCCC
TCCCCACACCGAAGCGGCTACTGCTCCGACATGGGGATCCTTCACCAGGGCTACTCCCTTAGCACAGG
GTCTGACGCCGACTCCGACACCGAGGGAGGGATGTCTCCAGAACACGCCATCAGACTGTGGGGCAGAG
GGATAAAATCCAGGCGCAGTTCCGGCCTGTCCAGTCGTGAAAACTCGGCCCTTACCCTGACTGACTCT
GACAACGAAAACAAATCAGATGATGAGAACGGTCGTCCCATTCCACCTACATCCTCGCCTAGTCTCCT
CCCATCTGCTCAGCTGCCTAGCTCCCATAATCCTCCACCAGTTAGCTGCCAGATGCCATTGCTAGACA
GCAACACCTCCCATCAAATCATGGACACCAACCCTGATGAGGAATTCTCCCCCAATTCATACCTGCTC
AGAGCATGCTCAGGGCCCCAGCAAGCCTCCAGCAGTGGCCCTCCGAACCACCACAGCCAGTCGACTCT
GAGGCCCCCTCTCCCACCCCCTCACAACCACACGCTGTCCCATCACCACTCGTCCGCCAACTCCCTCA
ACAGGAACTCACTGACCAATCGGCGGAGTCAGATCCACGCCCCGGCCCCAGCGCCCAATGACCTGGCC
ACCACACCAGAGTCCGTTCAGCTTCAGGACAGCTGGGTGCTAAACAGCAACGTGCCACTGGAGACCCG
GCACTTCCTCTTCAAGACCTCCTCGGGGAGCACACCCTTGTTCAGCAGCTCTTCCCCGGGATACCCTT
TGACCTCAGGAACGGTTTACACGCCCCCGCCCCGCCTGCTGCCCAGGAATACTTTCTCCAGGAAGGCT
TTCAAGCTGAAGAAGCCCTCCAAATACTGCAGCTGGAAATGTGCTGCCCTCTCCGCCATTGCCGCGGC
CCTCCTCTTGGCTATTTTGCTGGCGTATTTCATAGTGCCCTGGTCGTTGAAAAACAGCAGCATAGACA
GTGGTGAAGCAGAAGTTGGTCGGCGGGTAACACAAGAAGTCCCACCAGGGGTGTTTTGGAGGTCACAA
ATTCACATCAGTCAGCCCCAGTTCTTAAAGTTCAACATCTCCCTCGGGAAGGACGCTCTCTTTGGTGT
TTACATAAGAAGAGGACTTCCACCATCTCATGCCCAGTATGACTTCATGGAACGTCTGGACGGGAAGG
AGAAGTGGAGTGTGGTTGAGTCTCCCAGGGAACGCCGGAGCATACAGACCTTGGTTCAGAATGAAGCC
GTGTTTGTGCAGTACCTGGATGTGGGCCTGTGGCATCTGGCCTTCTACAATGATGGAAAAGACAAAGA
GATGGTTTCCTTCAATACTGTTGTCCTAGATTCAGTGCAGGACTGTCCACGTAACTGCCATGGGAATG
GTGAATGTGTGTCCGGGGTGTGTCACTGTTTCCCAGGATTTCTAGGAGCAGACTGTGCTAAAGCTGCC
TGCCCTGTCCTGTGCAGTGGGAATGGACAATATTCTAAAGGGACGTGCCAGTGCTACAGCGGCTGGAA
AGGTGCAGAGTGCGACGTGCCCATGAATCAGTGCATCGATCCTTCCTGCGGGGGCCACGGCTCCTGCA
TTGATGGGAACTGTGTCTGCTCTGCTGGCTACAAAGGCGAGCACTGTGAGGAAGTTGATTGCTTGGAT
CCCACCTGCTCCAGCCACGGAGTCTGTGTGAATGGAGAATGCCTGTGCAGCCCTGGCTGGGGTGGTCT
GAACTGTGAGCTGGCGAGGGTCCAGTGCCCAGACCAGTGCAGTGGGCATGGCACGTACCTGCCTGACA
CGGGCCTCTGCAGCTGCGATCCCAACTGGATGGGTCCCGACTGCTCTGTTGAAGTGTGCTCAGTAGAC
TGTGGCACTCACGGCGTCTGCATCGGGGGAGCCTGCCGCTGTGAAGAGGGCTGGACAGGCGCAGCGTG
TGACCAGCGCGTGTGCCACCCCCGCTGCATTGAGCATGGGACCTGTAAAGATGGCAAATGTGAATGCC
GAGAGGGCTGGAATGGTGAACACTGCACCATTGATGGCTGCCCTGACTTGTGCAACGGTAACGGGAGA
TGCACACTGGGTCAGAACAGCTGGCAGTGTGTCTGCCAGACCGGCTGGAGAGGGCCCGGATGCAACGT
TGCCATGGAAACTTCCTGTGCTGATAACAAGGATAATGAGGGAGATGGCCTGGTGGATTGTTTGGACC
CTGACTGCTGCCTGCAGTCAGCCTGTCAGAACAGCCTGCTCTGCCGGGGGTCCCGGGACCCACTGGAC
ATCATTCAGCAGGGCCAGACGGATTGGCCCGCAGTGAAGTCCTTCTATGACCGTATCAAGCTCTTGGC
AGGCAAGGATAGCACCCACATCATTCCTGGAGAGAACCCTTTCAACAGCAGCTTGGTTTCTCTCATCC
GAGGCCAAGTAGTAACTACAGATGGAACTCCCCTGGTCGGTGTGAACGTGTCTTTTGTCAAGTACCCA
AAATACGGCTACACCATCACCCGCCAGGATGGCACGTTCGACCTGATCGCAAATGGAGGTGCTTCCTT
GACTCTACACTTTGAGCGAGCCCCGTTCATGAGCCAGGAGCGCACTGTGTGGCTGCCGTGGAACAGCT
TTTACGCCATGGACACCCTGGTGATGAAGACCGAGGAGAACTCCATCCCCAGCTGTGACCTCAGTGGC
TTTGTCCGGCCTGATCCAATCATCATCTCCTCCCCACTGTCCACCTTCTTTAGTGCTGCCCCTGGGCA
GAATCCCATCGTGCCTGAGACCCAGGTTCTTCATGAAGAAATCGAGCTCCCTGGTTCCAATGTGAAAC
TTCGCTATCTGAGCTCTAGAACTGCAGGGTACAAGTCACTGCTGAAGATCACCATGACCCAGTCCACA
GTGCCCCTGAACCTCATTAGGGTTCACCTGATGGTGGCTGTCGAGGGGCATCTCTTCCAGAAGTCATT
CCAGGCTTCTCCCAACCTGGCCTCCACCTTCATCTGGGACAAGACAGATGCGTATGGCCAAAGGGTGT
ATGGACTCTCAGATGCTGTTGTGTCTGTCGGGTTTGAATATGAGACCTGTCCCAGTCTAATTCTCTGG
GAGAAAAGGACAGCCCTCCTTCAGGGATTCGAGCTGGACCCCTCCAACCTCGGTGGCTGGTCCCTAGA
CAAACACCACATCCTCAATGTTAAAAGTGGAATCCTACACAAAGGCACTGGGGAAAACCAGTTCCTGA
CCCAGCAGCCTGCCATCATCACCAGCATCATGGGCAATGGTCGCCGCCGGAGCATTTCCTGTCCCAGC
TGCAACGGCCTTGCTGAAGGCAACAAGCTGCTGGCCCCAGTGGCTCTGGCTGTTGGAATCGATGGGAG
CCTCTATGTGGGTGACTTCAATTACATCCGACGCATCTTTCCCTCTCGAAATGTGACCAGCATCTTGG
AGTTACGAAATAAAGAGTTTAAACATAGCAACAACCCAGCACACAAGTACTACTTGGCAGTGGACCCC
GTGTCCGGCTCGCTCTACGTGTCCGACACCAACAGCAGGAGAATCTACCGCGTCAAGTCTCTGAGTGG
AACCAAAGACCTGGCTGGGAATTCGGAAGTTGTGGCAGGGACGGGAGAGCAGTGTCTACCCTTTGATG
AAGCCCGCTGCGGGGATGGAGGGAAGGCCATAGATGCAACCCTGATGAGCCCGAGAGGTATTGCAGTA
GACAAGAATGGGCTCATGTACTTTGTCGATGCCACCATGATCCGGAAGGTTGACCAGAATGGAATCAT
CTCCACCCTGCTGGGCTCCAATGACCTCACTGCCGTCCGGCCGCTGAGCTGTGATTCCAGCATGGATG
TAGCCCAGGTTCGTCTGGAGTGGCCAACAGACCTTGCTGTCAATCCCATGGATAACTCCTTGTATGTT
CTAGAGAACAATGTCATCCTTCGAATCACCGAGAACCACCAAGTCAGCATCATTGCGGGACGCCCCAT
GCACTGCCAAGTTCCTGGCATTGACTACTCACTCAGCAAACTAGCCATTCACTCTGCCCTGGAGTCAG
CCAGTGCCATTGCCATTTCTCACACTGGGGTCCTCTACATCACTGAGACAGATGAGAAGAAGATTAAC
CGTCTACGCCAGGTAACAACCAACGGGGAGATCTGCCTTTTAGCTGGGGCAGCCTCGGACTGCGACTG
CAAAAACGATGTCAATTGCAACTGCTATTCAGGAGATGATGCCTACGCGACTGATGCCATCTTGAATT
CCCCATCATCCTTAGCTGTAGCTCCAGATGGTACCATTTACATTGCAGACCTTGGAAATATTCGGATC
AGGGCGGTCAGCAAGAACAAGCCTGTTCTTAATGCCTTCAACCAGTATGAGGCTGCATCCCCCGGAGA
GCAGGAGTTATATGTTTTCAACGCTGATGGCATCCACCAATACACTGTGAGCCTGGTGACAGGGGAGT
ACTTGTACAATTTCACATATAGTACTGACAATGATGTCACTGAATTGATTGACAATAATGGGAATTCC
CTGAAGATCCGTCGGGACAGCAGTGGCATGCCCCGTCACCTGCTCATGCCTGACAACCAGATCATCAC
CCTCACCGTGGGCACCAATGGAGGCCTCAAAGTCGTGTCCACACAGAACCTGGAGCTTGGTCTCATGA
CCTATGATGGCAACACTGGGCTCCTGGCCACCAAGAGCGATGAAACAGGATGGACGACTTTCTATGAC
TATGACCACGAAGGCCGCCTGACCAACGTGACGCGCCCCACGGGGGTGGTAACCAGTCTGCACCGGGA
AATGGAGAAATCTATTACCATTGACATTGAGAACTCCAACCGTGATGATGACGTCACTGTCATCACCA
ACCTCTCTTCAGTAGAGGCCTCCTACACAGTGGTACAAGATCAAGTTCGGAACAGCTACCAGCTCTGT
AATAATGGTACCCTGAGGGTGATGTATGCTAATGGGATGGGTATCAGCTTCCACAGCGAGCCCCATGT
CCTAGCGGGCACCATCACCCCCACCATTGGACGCTGCAACATCTCCCTGCCTATGGAGAATGGCTTAA
ACTCCATTGAGTGGCGCCTAAGAAAGGAACAGATTAAAGGCAAAGTCACCATCTTTGGCAGGAAGCTC
CGGGTCCATGGAAGAAATCTCTTGTCCATTGACTATGATCGAAATATTCGGACTGAAAAGATCTATGA
TGACCACCGGAAGTTCACCCTGAGGATCATTTATGACCAGGTGGGCCGCCCCTTCCTCTGGCTGCCCA
GCCATGAGCGAGAGGACAGACATCGACAAGCAAGGCCGCATCGTGTCCCGCATGTTCGCTGACGGGAA
AGTGTGGAGCTACTCCTACCTTGACAAGTCCATGGTCCTCCTGCTTCAGAGCCAACGTCAGTATATAT
TTGAGTATGACTCCTCTGACCGCCTCCTTGCCGTCACCATGCCCAGCGTGGCCCGGCACAGCATGTCC
ACACACACCTCCATCGGCTACATCCGTAATATTTACAACCCGCCTGAAAGCAATGCTTCGGTCATCTT
TGACTACAGTGATGACGGCCGCATCCTGAAGACCTCCTTTTTGGGCACCGGACGCCAGGTGTTCTACA
AGTATGGGAAACTCTCCAAGTTATCAGAGATTGTCTACGACAGTACCGCCGTCACCTTCGGGTATGAC
GAGACCACTGGTGTCTTGAAGATGGTCAACCTCCAAAGTGGGGGCTTCTCCTGCACCATCAGGTACCG
GAAGATTGGCCCCCTGGTGGACAAGCAGATCTACAGGTTCTCCGAGGAAGGCATGGTCAATGCCAGGT
TTGACTACACCTATCATGACAACAGCTTCCGCATCGCAAGCATCAAGCCCGTCATAAGTGAGACTCCC
CTCCCCGTTGACCTCTACCGCTATGATGAGATTTCTGGCAAGGTGGAACACTTTGGTAAGTTTGGAGT
CATCTATTATGACATCAACCAGATCATCACCACTGCCGTGATGACCCTCAGCAAACACTTCGACACCC
ATGGGCGGATCAAGGAGGTCCAGTATGAGATGTTCCGGTCCCTCATGTACTGGATGACGGTGCAATAT
GACAGCATGGGCAGGGTGATCAAGAGGGAGCTAAAACTGGGGCCCTATGCCAATACCACGAAGTACAC
CTATGACTACGATGGGGACGGGCAGCTCCAGAGCGTGGCCGTCAATGACCGCCCGACCTGGCGCTACA
GCTATGACCTTAATGGGAATCTCCACTTACTGAACCCAGGCAACAGTGTGCGCCTCATGCCCTTGCGC
TATGACCTCCGGGATCGGATAACCAGACTCGGGGATGTGCAGTACAAAATTGACGACGATGGCTATCT
GTGCCAGAGAGGGTCTGACATCTTCGAATACAATTCCAAGGGCCTCCTAACAAGAGCCTACAACAAGG
CCAGCGGGTGGAGTGTCCAGTACCGCTATGATGGCGTAGGACGGCGGGCTTCCTACAAGACCAACCTG
GGCCACCACCTGCAGTACTTCTACTCTGACCTCCACAACCCGACGCGCATCACCCATGTCTACAATCA
CTCCAACTCGGAGATTACCTCACTGTACTACGACCTCCAGGGCCACCTCTTTGCCATGGAGAGCAGCA
GTGGGGAGGAGTACTATGTTGCCTCTGATAACACAGGGACTCCTCTGGCTGTGTTCAGCATCAACGGC
CTCATGATCAAACAGCTGCAGTACACGGCCTATGGGGAGATTTATTATGACTCCAACCCCGACTTCCA
GATGGTCATTGGCTTCCATGGGGGACTCTATGACCCCCTGACCAAGCTGGTCCACTTCACTCAGCGTG
ATTATGATGTGCTGGCAGGACGATGGACCTCCCCAGACTATACCATGTGGAAAAACGTGGGCAAGGAG
CCGGCCCCCTTTAACCTGTATATGTTCAAGAGCAACAATCCTCTCAGCAGTGAGCTAGATTTGAAGAA
CTACGTGACAGATGTGAAAAGCTGGCTTGTGATGTTTGGATTTCAGCTTAGCAACATCATTCCTGGCT
TCCCGAGAGCCAAAATGTATTTCGTGCCTCCTCCCTATGAATTGTCAGAGAGTCAAGCAAGTGAGAAT
GGACAGCTCATTACAGGTGTCCAACAGACAACAGAGAGACATAACCAGGCCTTCATGGCTCTGGAAGG
ACAGGTCATTACTAAAAAGCTCCACGCCAGCATCCGAGAGAAAGCAGGTCACTGGTTTGCCACCACCA
CGCCCATCATTGGCAAAGGCATCATGTTTGCCATCAAAGAAGGGCGGGTGACCACGGGCGTGTCCAGC
ATCGCCAGCGAAGATAGCCGCAAGGTGGCATCTGTGCTGAACAACGCCTACTACCTGGACAAGATGCA
CTACAGCATCGAGGGCAAGGACACCCACTACTTTGTGAAGATTGGCTCAGCCGATGGCGACCTGGTCA
CACTAGGCACCACCATCGGCCGCAAGGTGCTAGAGAGCGGGGTGAACGTGACCGTGTCCCAGCCCACG
CTGCTGGTCAACGGCAGGACTCGAAGGTTCACGAACATTGAGTTCCAGTACTCCACGCTGCTGCTCAG
CATCCGCTATGGCCTCACCCCCGACACCCTGGACGAAGAGAAGGCCCGCGTCCTGGACCAGGCGAGAC
AGAGGGCCCTGGGCACGGCCTGGGCCAAGGAGCAGCAGAAAGCCAGGGACGGGAGAGAGGGGAGCCGC
CTGTGGACTGAGGGCGAGAAGCAGCAGCTTCTGAGCACCGGGCGCGTGCAAGGGTACGAGGGATATTA
CGTGCTTCCCGTGGAGCAATACCCAGAGCTTGCAGACAGTAGCAGCAACATCCAGTTTTTAAGACAGA
ATGAGATGGGAAAGAGGTAACAAAATAATCTGCTGCCATTCCTTGTCTGAATGGCTCAGCAGGAGTAA
CTGTTATCTCCTCTCCTAAGGAGATGAAGACCTAACAGGGGCACTGCGGCTGGGCTGCTTTAGGAGAC
CAAGTGGCAAGAAAGCTCACATTTTTTGAGTTCAAATGCTACTGTCCAAGCGAGAAGTCCCTCATCCT
GAAGTAGACTAAAGCCCGGCTGAAAATTCCGAGGAAAACAAAACAAACGAATGAATGAACAGACACAC
ACAATGTTCCAAGTTCCCCTAAAATATGACCCACTTGTTCTGGGTCTACGCAGAAAAGAGACGCAAAG
TGTCCAAAAGGAACAAAAGAACAAAAACGAATAAGCAAAGAAGAAAACAAACAAAAACAAAACAAAAC
AAACACACGGACCGATAAACAAAGAAGCGAAGATAAGAAAGAAGGCCTCATATCCAATTACCTCACTC
ATTCACATGTGAGCGACACGCAGACATCCGCGAGGGCCAGCGTCACCAGACCAGCTGCGGGACAAACC
ACTCAGACTGCTTGTAGGACAAATACTTCTGACATTTTCGTTTAAGCAAATACAGGTGCATTTAAAAC
ACGACTTTGGGGGTGATTTGTGTGTAGCGCCTGGGGAGGGGGGATAAAAGAGGAGGAGTGAGCACTGG
AAATACTTTTTAAAGAAAAAAAAACATGAGGGAATAAAAGAAATTCCTATCAAAAATCAAAGTGAAAT
AATACCATCCAGCACTTAACTCTCAGGTCCCAACTAAGTCTGGCCTGAGCTAATTTATTTGAGCGCAG
AGTGTAAAATTTAATTCAAAATGGTGGCTATAATCACTACAGATAAATTTCATACTCTTTTGTCTTTG
GAGATTCCATTGTGGACAGTAATACGCAGTTACAGGGTGTAGTCTGTTTAGATTCCGTAGTTCGTGGG
TATCAGTTTCGGTAGAGGTGCAGCATCGTGACACTTTTGCTAACAGGTACCACTTCTGATCACCCTGT
ACATACATGAGCCGAAAGGCACAATCACTGTTTCAGATTTAAAATTATTAGTGTGTTTGTTTGGTCCA
GAAACTGAGACAATCACATGACAGTCACCACGAGGAGAGAAAATTTAAAAAATAAAAATAAAAACAAA
AAAAATTTTAAAAATTAAAAAAACAAAAATAAAGTCTAATAAGAACTTTGGTACAGGAACTTTTTTGT
AATATACATGTATGAATTGTTCATCGAGTTTTTATATTAATTTTAATTTGCTGCTAAGCAAAGACTAG
GGACAGGCAAAGATAATTTATGGCAAAGTGTTTAAATTGTTTATACATAAATAAAGTCTCTAAAACTC
CTGTG

| | | | |
|---|---|---|---|
| NOV7r, CG50426-11 | SEQ ID NO: 148 | 2724 aa | MW at 302521.3kD |
| Protein Sequence | | | |

MDVKDRRHRSLTRGRCGKECRYTSSSLDSEDCRVPTQKSYSSSETLKAYDHDSRMHYGNRVTDLIHRE
SDEFPRQGTNFTLAELGICEPSPHRSGYCSDMGILHQGYSLSTGSDADSDTEGGMSPEHAIRLWGRGI
KSRRSSGLSSRENSALTLTDSDNENKSDDENGRPIPPTSSPSLLPSAQLPSSHNPPPVSCQMPLLDSN
TSHQIMDTNPDEEFSPNSYLLRACSGPQQASSSGPPNHHSQSTLRPPLPPPHNHTLSHHHSSANSLNR
NSLTNRRSQIHAPAPAPNDLATTPESVQLQDSWVLNSNVPLETRHFLFKTSSGSTPLFSSSSPGYPLT
SGTVYTPPPRLLPRNTFSRKAFKLKKPSKYCSWKCAALSAIAAALLLAILLAYFIVPWSLKNSSIDSG
EAEVGRRVTQEVPPGVFWRSQIHISQPQFLKFNISLGKDALFGVYIRRGLPPSHAQYDFMERLDGKEK
WSVVESPRERRSIQTLVQNEAVFVQYLDVGLWHLAFYNDGKDKEMVSFNTVVLDSVQDCPRNCHGNGE
CVSGVCHCFPGFLGADCAKAACPVLCSGNGQYSKGTCQCYSGWKGAECDVPMNQCIDPSCGGHGSCID
GNCVCSAGYKGEHCEEVDCLDPTCSSHGVCVNGECLCSPGWGGLNCELARVQCPDQCSGHGTYLPDTG
LCSCDPNWMGPDCSVEVCSVDCGTHGVCIGGACRCEEGWTGAACDQRVCHPRCIEHGTCKDGKCECRE
GWNGEHCTIDGCPDLCNGNGRCTLGQNSWQCVCQTGWRGPGCNVAMETSCADNKDNEGDGLVDCLDPD
CCLQSACQNSLLCRGSRDPLDIIQQGQTDWPAVKSFYDRIKLLAGKDSTHIIPGENPFNSSLVSLIRG
QVVTTDGTPLVGVNVSFVKYPKYGYTITRQDGTFDLIANGGASLTLHFERAPFMSQERTVWLPWNSFY
AMDTLVMKTEENSIPSCDLSGFVRPDPIIISSPLSTFFSAAPGQNPIVPETQVLHEEIELPGSNVKLR
YLSSRTAGYKSLLKITMTQSTVPLNLIRVHLMVAVEGHLFQKSFQASPNLASTFIWDKTDAYGQRVYG
LSDAVVSVGFEYETCPSLILWEKRTALLQGFELDPSNLGGWSLDKHHILNVKSGILHKGTGENQFLTQ
QPAIITSIMGNGRRRSISCPSCNGLAEGNKLLAPVALAVGIDGSLYVGDFNYIRRIFPSRNVTSILEL
RNKEFKHSNNPAHKYYLAVDPVSGSLYVSDTNSRRIYRVKSLSGTKDLAGNSEVVAGTGEQCLPFDEA
RCGDGGKAIDATLMSPRGIAVDKNGLMYFVDATMIRKVDQNGIISTLLGSNDLTAVRPLSCDSSMDVA
QVRLEWPTDLAVNPMDNSLYVLENNVILRITENHQVSIIAGRPMHCQVPGIDYSLSKLAIHSALESAS
AIAISHTGVLYITETDEKKINRLRQVTTNGEICLLAGAASDCDCKNDVNCNCYSGDDAYATDAILNSP
SSLAVAPDGTIYIADLGNIRIRAVSKNKPVLNAFNQYEAASPGEQELYVFNADGIHQYTVSLVTGEYL
YNFTYSTDNDVTELIDNNGNSLKIRRDSSGMPRHLLMPDNQIITLTVGTNGGLKVVSTQNLELGLMTY
DGNTGLLATKSDETGWTTFYDYDHEGRLTNVTRPTGVVTSLHREMEKSITIDIENSNRDDDVTVITNL
SSVEASYTVVQDQVRNSYQLCNNGTLRVMYANGMGISFHSEPHVLAGTITPTIGRCNISLPMENGLNS
IEWRLRKEQIKGKVTIFGRKLRVHGRNLLSIDYDRNIRTEKIYDDHRKFTLRIIYDQVGRPFLWLPSS
GLAAVNVSYFFNGRLAGLQRGAMSERTDIDKQGRIVSRMFADGKVWSYSYLDKSMVLLLQSQRQYIFE
YDSSDRLLAVTMPSVARHSMSTHTSIGYIRNIYNPPESNASVIFDYSDDGRILKTSFLGTGRQVFYKY
GKLSKLSEIVYDSTAVTFGYDETTGVLKMVNLQSGGFSCTIRYRKIGpLVDKQIYRFSEEGMVNARFD
YTYHDNSFRIASIKPVISETPLPVDLYRYDEISGKVEHFGKFGVIYYDINQIITTAVMTLSKHFDTHG
RIKEVQYEMFRSLMYWMTVQYDSMGRVIKRELKLGPYANTTKYTYDYDGDGQLQSVAVNDRPTWRYSY
DLNGNLHLLNPGNSVRLMPLRYDLRDRITRLGDVQYKIDDDGYLCQRGSDIFEYNSKGLLTRAYNKAS
GWSVQYRYDGVGRRASYKTNLGHHLQYFYSDLHNPTRITHVYNHSNSEITSLYYDLQGHLFAMESSSG
EEYYVASDNTGTPLAVFSINGLMIKQLQYTAYGEIYYDSNPDFQMVIGFHGGLYDPLTKLVHFTQRDY
DVLAGRWTSPDYTMWKNVGKEPAPFNLYMFKSNNPLSSELDLKNYVTDVKSWLVMFGFQLSNIIPGFP
RAKMYFVPPPYELSESQASENGQLITGVQQTTERHNQAFMALEGQVITKKLHASIREKAGHWFATTTP
IIGKGIMFAIKEGRVTTGVSSIASEDSRKVASVLNNAYYLDKMHYSIEGKDTHYFVKIGSADGDLVTL
GTTIGRKVLESGVNVTVSQPTLLVNGRTRRFTNIEFQYSTLLLSIRYGLTPDTLDEEKARVLDQARQR
ALGTAWAKEQQKARDGREGSRLWTEGEKQQLLSTGRVQGYEGYYVLPVEQYPELADSSSNIQFLRQNE
MGKR

| | | |
|---|---|---|
| NOV7s, CG50426-12 | SEQ ID NO: 149 | 9826 bp |
| DNA Sequence | ORF Start: ATG at 280 | ORF Stop: TAA at 8479 |

TTTAAATCCTCATACCTTAAAGGAGATGTGTATATAAGGGAGTTGGAACCAGCATTAGATGAGTTGAC
AAAAATGCAGTTTCAGTTCTAGAGGTCTGGGAAGTCCAAGAACAAGGTGCTGGCAGATTGGATTCCCC
GTGAGGGCTTTCTTCCTGGCTTGAAGTTGGCTGCTTTCCTGCTGAGACTTCTCATGGCAGAGACTGAG
GGTGGCAAAGTGACAAGTGCCAAAACTCAGGCCTGACTTTTCTGAAAACATCAGCATTCTGCCATATC
TGGAATAATGGATGTAAAGGACCGGCGACACCGCTCTTTGACCAGAGGACGCTGTGGCAAAGAGTGTC
GCTACACAAGCTCCTCTCTGGACAGTGAGGACTGCCGGGTGCCCACACAGAAATCCTACAGCTCCAGT
GAGACTCTGAAGGCCTATGACCATGACAGCAGGATGCACTATGGAAACCGAGTCACAGACCTCATCCA
CCGGGAGTCAGATGAGTTTCCTAGACAAGGAACCAACTTCACCCTTGCCGAACTGGGCATCTGTGAGC
CCTCCCCACACCGAAGCGGCTACTGCTCCGACATGGGGATCCTTCACCAGGGCTACTCCCTTAGCACA
GGGTCTGACGCCGACTCCGACACCGAGGGAGGGATGTCTCCAGAACACGCCATCAGACTGTGGGGCAG
AGGGATAAAATCCAGGCGCAGTTCCGGCCTGTCCAGTCGTGAAAACTCGGCCCTTACCCTGACTGACT
CTGACAACGAAAACAAATCAGATGATGAGAACGGTCGTCCCATTCCACCTACATCCTCGCCTAGTCTC
CAGCAACACCTCCCATCAAATCATGGACACCAACCCTGATGAGGAATTCTCCCCCAATTCATACCTGC
TCAGAGCATGCTCAGGGCCCCAGCAAGCCTCCAGCAGTGGCCCTCCGAACCACCACAGCCAGTCGACT
CTGAGGCCCCCTCTCCCACCCCCTCACAACCACACGCTGTCCCATCACCACTCGTCCGCCAACTCCCT
CAACAGGAACTCACTGACCAATCGGCGGAGTCAGATCCACGCCCCGGCCCCAGCGCCCAATGACCTGG
CCACCACACCAGAGTCCGTTCAGCTTCAGGACAGCTGGGTGCTAAACAGCAACGTGCCACTGGAGACC
CGGCACTTCCTCTTCAAGACCTCCTCGGGGAGCACACCCTTGTTCAGCAGCTCTTCCCCGGGATACCC
TTTGACCTCAGGAACGGTTTACACGCCCCCGCCCCGCCTGCTGCCCAGGAATACTTTCTCCAGGAAGG
CTTTCAAGCTGAAGAAGCCCTCCAAATACTGCAGCTGGAAATGTGCTGCCCTCTCCGCCATTGCCGCG
GCCCTCCTCTTGGCTATTTTGCTGGCGTATTTCATAGTGCCCTGGTCGTTGAAAAACAGCAGCATAGA
CAGTGGTGAAGCAGAAGTTGGTCGGCGGGTAACACAAGAAGTCCCACCAGGGGTGTTTTGGAGGTCAC
AAATTCACATCAGTCAGCCCCAGTTCTTAAAGTTCAACATCTCCCTCGGGAAGGACGCTCTCTTTGGT
GTTTACATAAGAAGAGGACTTCCACCATCTCATGCCCAGTATGACTTCATGGAACGTCTGGACGGGAA
GGAGAAGTGGAGTGTGGTTGAGTCTCCCAGGGAACGCCGGAGCATACAGACCTTGGTTCAGAATGAAG
CCGTGTTTGTGCAGTACCTGGATGTGGGCCTGTGGCATCTGGCCTTCTACAATGATGGAAAAGACAAA
GAGATGGTTTCCTTCAATACTGTTGTCCTAGATTCAGTGCAGGACTGTCCACGTAACTGCCATGGGAA
TGGTGAATGTGTGTCCGGGGTGTGTCACTGTTTCCCAGGATTTCTAGGAGCAGACTGTGCTAAAGCTG
CCTGCCCTGTCCTGTGCAGTGGGAATGGACAATATTCTAAAGGGACGTGCCAGTGCTACAGCGGCTGG
AAAGGTGCAGAGTGCGACGTGCCCATGAATCAGTGCATCGATCCTTCCTGCGGGGGCCACGGCTCCTG
CATTGATGGGAACTGTGTCTGCTCTGCTGGCTACAAAGGCGAGCACTGTGAGGAAGTTGATTGCTTGG
ATCCCACCTGCTCCAGCCACGGAGTCTGTGTGAATGGAGAATGCCTGTGCAGCCCTAACTGGGGTGGT
CTGAACTGTGAGCTGGCGAGGGTCCAGTGCCCAGACCAGTGCAGTGGGCATGGCACGTACCTGCCTGA
CACGGGCCTCTGCAGCTGCGATCCCAACTGGATGGGTCCCGACTGCTCTGTTGAAGTGTGCTCAGTAG
ACTGTGGCACTCACGGCGTCTGCATCGGGGGAGCCTGCCGCTGTGAAGAGGGCTGGACAGGCGCAGCG
TGTGACCAGCGCGTGTGCCACCCCCGCTGCATTGAGCACGGGACCTGTAAAGATGGCAAATGTGAATG
CCGAGAGGGCTGGAATGGTGAACACTGCACCATTGGTAGGCAAACGGCAGGCACCGAAACAGATGGCT
GCCCTGACTTGTGCAACGGTAACGGGAGATGCACACTGGGTCAGAACAGCTGGCAGTGTGTCTGCCAG
ACCGGCTGGAGAGGGCCCGGATGCAACGTTGCCATGGAAACTTCCTGTGCTGATAACAAGGATAATGA
GGGAGATGGCCTGGTGGATTGTTTGGACCCTGACTGCTGCCTGCAGTCAGCCTGTCAGAACAGCCTGC
TCTGCCGGGGGTCCCGGGACCCACTGGACATCATTCAGCAGGGCCAGACGGATTGGCCCAAAGTGAAG
TCCTTCTATGACCGTATCAAGCTCTTGGCAGGCAAGGATAGCACCCACATCATTCCTGGAGAGAACCC
TTTCAACAGCAGCTTGGTTTCTCTCATCCGAGGCCAAGTAGTAACTACAGATGGAACTCCCCTGGTCG
GTGTGAACGTGTCTTTTGTCAAGTACCCAAAATACGGCTACACCATCACCCGCCAGGATGGCACGTTC
GACCTGATCGCAAATGGAGGTGCTTCCTTGACTCTACACTTTGAGCGAGCCCCGTTCATGAGCCAGGA
GCGCACTGTGTGGCTGCCGTGGAACAGCTTTTACGCCATGGACACCCTGGTGATGAAGACCGAGGAGA
ACTCCATCCCCAGCTGTGACCTCAGTGGCTTTGTCCGGCCTGATCCAATCATCATCTCCTCCCCACTG
TCCACCTTCTTTAGTGCTGCCCCTGGGCAGAATCCCATCGTGCCTGAGACCCAGGTTCTTCATGAAGA
AATCGAGCTCCCTGGTTCCAATGTGAAACTTCGCTATCTGAGCTCTAGAACTGCAGGGTACAAGTCAC
TGCTGAAGATCACCATGACCCAGTCCACAGTGCCCCTGAACCTCATTAGGGTTCACCTGATGGTGGCT
GTCGAGGGGCATCTCTTCCAGAAGTCATTCCAGGCTTCTCCCAACCTGGCCTCCACCTTCATCTGGGA
CAAGACAGATGCGTATGGCCAAAGGGTGTATGGACTCTCAGATGCTGTTGTGTCTGTCGGGTTTGAAT
ATGAGACCTGTCCCAGTCTAATTCTCTGGGAGAAAAGGACAGCCCTCCTTCAGGGATTCGAGCTGGAC
CCCTCCAACCTCGGTGGCTGGTCCCTAGACAAACACCACATCCTCAATGTTAAAAGTGGAATCCTACA
CAAAGGCACTGGGGAAAACCAGTTCCTGACCCAGCAGCCTGCCATCATCACCAGCATCATGGGCAATG
GTCGCCGCCGGAGCATTTCCTGTCCCAGCTGCAACGGCCTTGCTGAAGGCAACAAGCTGCTGGCCCCA
GTGGCTCTGGCTGTTGGAATCGATGGGAGCCTCTATGTGGGTGACTTCAATTACATCCGACGCATCTT
TCCCTCTCGAAATGTGACCAGCATCTTGGAGTTACGAAATAAAGAGTTTAAACATAGCAACAACCCAG
CACACAAGTACTACTTGGCAGTGGACCCCGTGTCCGGCTCGCTCTACGTGTCCGACACCAACAGCAGG
AGAATCTACCGCGTCAAGTCTCTGAGTGGAACCAAAGACCTGGCTGGGAATTCGGAAGTTGTGGCAGG
GACGGGAGAGCAGTGTCTACCCTTTGATGAAGCCCGCTGCGGGGATGGAGGGAAGGCCATAGATGCAA
CCCTGATGAGCCCGAGAGGTATTGCAGTAGACAAGAATGGGCTCATGTACTTTGTCGATGCCACCATG
ATCCGGAAGGTTGACCAGAATGGAATCATCTCCACCCTGCTGGGCTCCAATGACCTCACTGCCGTCCG
GCCGCTGAGCTGTGATTCCAGCATGGATGTAGCCCAGGTTCGTCTGGAGTGGCCAACAGACCTTGCTG
TCAATCCCATGGATAACTCCTTGTATGTTCTAGAGAACAATGTCATCCTTCGAATCACCGAGAACCAC
CAAGTCAGCATCATTGCGGGACGCCCCATGCACTGCCAAGTTCCTGGCATTGACTACTCACTCAGCAA
ACTAGCCATTCACTCTGCCCTGGAGTCAGCCAGTGCCATTGCCATTTCTCACACTGGGGTCCTCTACA
TCACTGAGACAGATGAGAAGAAGATTAACCGTCTACGCCAGGTAACAACCAACGGGGAGATCTGCCTT
TTAGCTGGGGCAGCCTCGGACTGCGACTGCAAAAACGATGTCAATTGCAACTGCTATTCAGGAGATGA
TGCCTACGCGACTGATGCCATCTTGAATTCCCCATCATCCTTAGCTGTAGCTCCAGATGGTACCATTT
ACATTGCAGACCTTGGAAATATTCGGATCAGGGCGGTCAGCAAGAACAAGCCTGTTCTTAATGCCTTC
AACCAGTATGAGGCTGCATCCCCCGGAGAGCAGGAGTTATATGTTTTCAACGCTGATGGCATCCACCA
ATACACTGTGAGCCTGGTGACAGGGGAGTACTTGTACAATTTCACATATAGTACTGACAATGATGTCA
CTGAATTGATTGACAATAATGGGAATTCCCTGAAGATCCGTCGGGACAGCAGTGGCATGCCCCGTCAC
CTGCTCATGCCTGACAACCAGATCATCACCCTCACCGTGGGCACCAATGGAGGCCTCAAAGTCGTGTC
CACACAGAACCTGGAGCTTGGTCTCATGACCTATGATGGCAACACTGGGCTCCTGGCCACCAAGAGCG
ATGAAACAGGATGGACGACTTTCTATGACTATGACCACGAAGGCCGCCTGACCAACGTGACGCGCCCC
ACGGGGGTGGTAACCAGTCTGCACCGGGAAATGGAGAAATCTATTACCATTGACATTGAGAACTCCAA
CCGTGATGATGACGTCACTGTCATCACCAACCTCTCTTCAGTAGAGGCCTCCTACACAGTGGTACAAG
ATCAAGTTCGGAACAGCTACCAGCTCTGTAATAATGGTACCCTGAGGGTGATGTATGCTAATGGGATG
GGTATCAGCTTCCACAGCGAGCCCCATGTCCTAGCGGGCACCATCACCCCCACCATTGGACGCTGCAA
CATCTCCCTGCCTATGGAGAATGGCTTAAACTCCATTGAGTGGCGCCTAAGAAAGGAACAGATTAAAG
GCAAAGTCACCATCTTTGGCAGGAAGCTCCGGGTCCATGGAAGAAATCTCTTGTCCATTGACTATGAT
CGAAATATTCGGACTGAAAAGATCTATGATGACCACCGGAAGTTCACCCTGAGGATCATTTATGACCA
GGTGGGCCGCCCCTTCCTCTGGCTGCCCAGCAGCGGGCTGGCAGCTGTCAACGTGTCATACTTCTTCA
ATGGGCGCCTGGCTGGGCTTCAGCGTGGGGCCATGAGCGAGAGGACAGACATCGACAAGCAAGGCCGC
ATCGTGTCCCGCATGTTCGCTGACGGGAAAGTGTGGAGCTACTCCTACCTTGACAAGTCCATGGTCCT
CCTGCTTCAGAGCCAACGTCAGTATATATTTGAGTATGACTCCTCTGACCGCCTCCTTGCCGTCACCA
TGCCCAGCGTGGCCCGGCACAGCATGTCCACACACACCTCCATCGGCTACATCCGTAATATTTACAAC
CCGCCTGAAAGCAATGCTTCGGTCATCTTTGACTACAGTGATGACGGCCGCATCCTGAAGACCTCCTT
TTTGGGCACCGGACGCCAGGTGTTCTACAAGTATGGGAAACTCTCCAAGTTATCAGAGATTGTCTACG
ACAGTACCGCCGTCACCTTCGGGTATGACGAGACCACTGGTGTCTTGAAGATGGTCAACCTCCAAAGT
GGGGGCTTCTCCTGCACCATCAGGTACCGGAAGATTGGCCCCCTGGTGGACAAGCAGATCTACAGGTT
CTCCGAGGAAGGCATGGTCAATGCCAGGTTTGACTACACCTATCATGACAACAGCTTCCGCATCGCAA
GCATCAAGCCCGTCATAAGTGAGACTCCCCTCCCCGTTGACCTCTACCGCTATGATGAGATTTCTGGC
AAGGTGGAACACTTTGGTAAGTTTGGAGTCATCTATTATGACATCAACCAGATCATCACCACTGCCGT
GATGACCCTCAGCAAACACTTCGACACCCATGGGCGGATCAAGGAGGTCCAGTATGAGATGTTCCGGT
CCCTCATGTACTGGATGACGGTGCAATATGACAGCATGGGCAGGGTGATCAAGAGGGAGCTAAAACTG
GGGCCCTATGCCAATACCACGAAGTACACCTATGACTACGATGGGGACGGGCAGCTCCAGAGCGTGGC
CGTCAATGACCGCCCGACCTGGCGCTACAGCTATGACCTTAATGGGAATCTCCACTTACTGAACCCAG
GCAACAGTGTGCGCCTCATGCCCTTGCGCTATGACCTCCGGGATCGGATAACCAGACTCGGGGATGTG
CAGTACAAAATTGACGACGATGGCTATCTGTGCCAGAGAGGGTCTGACATCTTCGAATACAATTCCAA
GGGCCTCCTAACAAGAGCCTACAACAAGGCCAGCGGGTGGAGTGTCCAGTACCGCTATGATGGCGTAG
GACGGCGGGCTTCCTACAAGACCAACCTGGGCCACCACCTGCAGTACTTCTACTCTGACCTCCACAAC
CCGACGCGCATCACCCATGTCTACAATCACTCCAACTCGGAGATTACCTCACTGTACTACGACCTCCA
GGGCCACCTCTTTGCCATGGAGAGCAGCAGTGGGGAGGAGTACTATGTTGCCTCTGATAACACAGGGA
CTCCTCTGGCTGTGTTCAGCATCAACGGCCTCATGATCAAACAGCTGCAGTACACGGCCTATGGGGAG
ATTTATTATGACTCCAACCCCGACTTCCAGATGGTCATTGGCTTCCATGGGGGACTCTATGACCCCCT
GACCAAGCTGGTCCACTTCACTCAGCGTGATTATGATGTGCTGGCAGGACGATGGACCTCCCCAGACT
ATACCATGTGGAAAAACGTGGGCAAGGAGCCGGCCCCCTTTAACCTGTATATGTTCAAGAGCAACAAT
CCTCTCAGCAGTGAGCTAGATTTGAAGAACTACGTGACAGATGTGAAAAGCTGGCTTGTGATGTTTGG
ATTTCAGCTTAGCAACATCATTCCTGGCTTCCCGAGAGCCAAAATGTATTTCGTGCCTCCTCCCTATG
AATTGTCAGAGAGTCAAGCAAGTGAGAATGGACAGCTCATTACAGGTGTCCAACAGACAACAGAGAGA
CATAACCAGGCCTTCATGGCTCTGGAAGGACAGGTCATTACTAAAAAGCTCCACGCCAGCATCCGAGA
GAAAGCAGGTCACTGGTTTGCCACCACCACGCCCATCATTGGCAAAGGCATCATGTTTGCCATCAAAG
AAGGGCGGGTGACCACGGGCGTGTCCAGCATCGCCAGCGAAGATAGCCGCAAGGTGGCATCTGTGCTG
AACAACGCCTACTACCTGGACAAGATGCACTACAGCATCGAGGGCAAGGACACCCACTACTTTGTGAA
GATTGGCTCAGCCGATGGCGACCTGGTCACACTAGGCACCACCATCGGCCGCAAGGTGCTAGAGAGCG
GGGTGAACGTGACCGTGTCCCAGCCCACGCTGCTGGTCAACGGCAGGACTCGAAGGTTCACGAACATT
GAGTTCCAGTACTCCACGCTGCTGCTCAGCATCCGCTATGGCCTCACCCCCGACACCCTGGACGAAGA
GAAGGCCCGCGTCCTGGACCAGGCGAGACAGAGGGCCCTGGGCACGGCCTGGGCCAAGGAGCAGCAGA
AAGCCAGGGACGGGAGAGAGGGGAGCCGCCTGTGGACTGAGGGCGAGAAGCAGCAGCTTCTGAGCACC
GGGCGCGTGCAAGGGTACGAGGGATATTACGTGCTTCCCGTGGAGCAATACCCAGAGCTTGCAGACAG
TAGCAGCAACATCCAGTTTTTAAGACAGAATGAGATGGGAAAGAGGTAACAAAATAATCTGCTGCCAT
TCCTTGTCTGAATGGCTCAGCAGGAGTAACTGTTATCTCCTCTCCTAAGGAGATGAAGACCTAACAGG
GGCACTGCGGCTGGGCTGCTTTAGGAGACCAAGTGGCAAGAAAGCTCACATTTTTTGAGTTCAAATGC
TACTGTCCAAGCGAGAAGTCCCTCATCCTGAAGTAGACTAAAGCCCGGCTGAAAATTCCGAGGAAAAC
AAAACAAACGAATGAATGAACAGACACACACAATGTTCCAAGTTCCCCTAAAATATGACCCACTTGTT
CTGGGTCTACGCAGAAAAGAGACGCAAAGTGTCCAAAAGGAACAAAAGAACAAAAACGAATAAGCAAA
GAAGAAAACAAACAAAAACAAAACAAAACAAACACACGGACCGATAAACAAAGAAGCGAAGATAAGAA
AGAAGGCCTCATATCCAATTACCTCACTCATTCACATGTGAGCGACACGCAGACATCCGCGAGGGCCA
GCGTCACCAGACCAGCTGCGGGACAAACCACTCAGACTGCTTGTAGGACAAATACTTCTGACATTTTC
GTTTAAGCAAATACAGGTGCATTTAAAACACGACTTTGGGGGTGATTTGTGTGTAGCGCCTGGGGAGG
GGGGATAAAAGAGGAGGAGTGAGCACTGGAAATACTTTTTAAAGAAAAAAAAACATGAGGGAATAAAA
GAAATTCCTATCAAAAATCAAAGTGAAATAATACCATCCAGCACTTAACTCTCAGGTCCCAACTAAGT
CTGGCCTGAGCTAATTTATTTGAGCGCAGAGTGTAAAATTTAATTCAAAATGGTGGCTATAATCACTA
CAGATAAATTTCATACTCTTTTGTCTTTGGAGATTCCATTGTGGACAGTAATACGCAGTTACAGGGTG
TAGTCTGTTTAGATTCCGTAGTTCGTGGGTATCAGTTTCGGTAGAGGTGCAGCATCGTGACACTTTTG
CTAACAGGTACCACTTCTGATCACCCTGTACATACATGAGCCGAAAGGCACAATCACTGTTTCAGATT
TAAAATTATTAGTGTGTTTGTTTGGTCCAGAAACTGAGACAATCACATGACAGTCACCACGAGGAGAG
AAAATTTAAAAAATAAAAATAAAAACAAAAAAAATTTTAAAAATTAAAAAAACAAAAATAAAGTCTAA
TAAGAACTTTGGTACAGGAACTTTTTTGTAATATACATGTATGAATTGTTCATCGAGTTTTTATATTA
ATTTTAATTTGCTGCTAAGCAAAGACTAGGGACAGGCAAAGATAATTTATGGCAAAGTGTTTAAATTG
TTTATACATAAATAAAGTCTCTAAAACTCCTGTG

| | | | |
|---|---|---|---|
| NOV7s, CG50426-12 | SEQ ID NO: 150 | 2733 aa | MW at 303423.3kD |
| Protein Sequence | | | |

MDVKDRRHRSLTRGRCGKECRYTSSSLDSEDCRVPTQKSYSSSETLKAYDHDSRMHYGNRVTDLIHRE
SDEFPRQGTNFTLAELGICEPSPHRSGYCSDMGILHQGYSLSTGSDADSDTEGGMSPEHAIRLWGRGI
KSRRSSGLSSRENSALTLTDSDNENKSDDENGRPIPPTSSPSLLPSAQLPSSHNPPPVSCQMPLLDSN
TSHQIMDTNPDEEFSPNSYLLRACSGPQQASSSGPPNHHSQSTLRPPLPPPHNHTLSHHHSSANSLNR
NSLTNRRSQIHAPAPAPNDLATTPESVQLQDSWVLNSNVPLETRHFLFKTSSGSTPLFSSSSPGYPLT
SGTVYTPPPRLLPRNTFSRKAFKLKKPSKYCSWKCAALSAIAAALLLAILLAYFIVPWSLKNSSIDSG
EAEVGRRVTQEVPPGVFWRSQIHISQPQFLKFNISLGKDALFGVYIRRGLPPSHAQYDFMERLDGKEK
WSVVESPRERRSIQTLVQNEAVFVQYLDVGLWHLAFYNDGKDKEMVSFNTVVLDSVQDCPRNCHGNGE
CVSGVCHCFPGFLGADCAKAACPVLCSGNGQYSKGTCQCYSGWKGAECDVPMNQCIDPSCGGHGSCID
GNCVCSAGYKGEHCEEVDCLDPTCSSHGVCVNGECLCSPGWGGLNCELARVQCPDQCSGHGTYLPDTG
LCSCDPNWNGPDCSVEVCSVDCGTHGVCIGGACRCEEGWTGAACDQRVCHPRCIEHGTCKDGKCECRE
GWNGEHCTIGRQTAGTETDGCPDLCNGNGRCTLGQNSWQCVCQTGWRGPGCNVAMETSCADNKDNEGD
GLVDCLDPDCCLQSACQNSLLCRGSRDPLDIIQQGQTDWPAVKSFYDRIKLLAGKDSTHIIPGENPFN
SSLVSLIRGQVVTTDGTPLVGVNVSFVKYPKYGYTITRQDGTFDLIANGGASLTLHFERAPFMSQERT
VWLPWNSFYAMDTLVMKTEENSIPSCDLSGFVRPDPIIISSPLSTFFSAAPGQNPIVPETQVLHEEIE
LPGSNVKLRYLSSRTAGYKSLLKITMTQSTVPLNLIRVHLMVAVEGHLFQKSFQASPNLASTFIWDKT
DAYGQRVYGLSDAVVSVGFEYETCPSLILWEKRTALLQGFELDPSNLGGWSLDKHHILNVKSGILHKG
TGENQFLTQQPAIITSIMGNGRRRSISCPSCNGLAEGNKLLAPVALAVGIDGSLYVGDFNYIRRIFPS
RNVTSILELRNKEFKHSNNPAHKYYLAVDPVSGSLYVSDTNSRRIYRVKSLSGTKDLAGNSEVVAGTG
EQCLPFDEARCGDGGKAIDATLMSPRGIAVDKNGLMYFVDATMIRKVDQNGIISTLLGSNDLTAVRPL
SCDSSMDVAQVRLEWPTDLAVNPMDNSLYVLENNVILRITENHQVSIIAGRPMHCQVPGIDYSLSKLA
IHSALESASAIAISHTGVLYITETDEKKINRLRQVTTNGEICLLAGAASDCDCKNDVNCNCYSGDDAY
ATDAILNSPSSLAVAPDGTIYIADLGNIRIRAVSKNKPVLNAYNQYEAASPGEQELYVFNADGIHQYT
VSLVTGEYLYNFTYSTDNDVTELIDNNGNSLKIRRDSSGMPRHLLMPDNQIITLTVGTNGGLKVVSTQ
NLELGLMTYDGNTGLLATKSDETGWTTFYDYDHEGRITNVTRPTGVVTSLHREMEKSITIDIENSNRD
DDVTVITNLSSVEASYTVVQDQVRNSYQLCNNGTLRVMYANGMGISFHSEPHVLAGTITPTIGRCNIS
LPMENGLNSIEWRLRKEQIKGKVTIFGRKLRVHGRNLLSIDYDRNIRTEKIYDDHRKFTLRIIYDQVG
RPFLWLPSSGLAAVNVSYFFNGRLAGLQRGAMSERTDIDKQGRIVSRMFADGKVWSYSYLDKSMVLLL
QSQRQYIFEYDSSDRLLAVTMPSVARHSMSTHTSIGYIRNIYNPPESNASVIFDYSDDGRILKTSFLG
TGRQVFYKYGKLSKLSEIVYDSTAVTFGYDETTGVLKMVNLQSGGFSCTIRYRKIGPLVDKQIYRFSE
EGMVNARFDYTYHDNSFRIASIKPVISETPLPVDLYRYDEISGKVENFGKFGVTYYDINQIITTAVMT
LSKHFDTHGRIKEVQYEMFRSLMYWMTVQYDSMGRVIKRELKLGPYANTTKYTYDYDGDGQLQSVAVN
DRPTWRYSYDLNGNLHLLNPGNSVRLMPLRYDLRDRITRLGDVQYKIDDDGYLCQRGSDIFEYNSKGL
LTRAYNKASGWSVQYRYDGVGRRASYKTNLGHHLQYFYSDLHNPTRITHVYNHSNSEITSLYYDLQGH
LFAMESSSGEEYYVASDNTGTPLAVFSINGLMIKQLQYTAYGEIYYDSNPDFQMVIGFHGGLYDPLTK
LVHFTQRDYDVLAGRWTSPDYTMWKNVGKEPAPFNLYMFKSNNPLSSELDLKNYVTDVKSWLVMFGFQ
LSNIIPGFPRAKMYFVPPPYELSESQASENGQLITGVQQTTERHNQAFMALEGQVITKKLHASIREKA
GHWFATTTPIIGKGIMFAIKEGRVTTGVSSIASEDSRKVASVLNNAYYLDKMHYSIEGKDTHYFVKIG
SADGDLVTLGTTIGRKVLESGVNVTVSQPTLLVNGRTRPFTNIEFQYSTLLLSIRYGLTPDTLDEEKA
RVLDQARQRALGTAWAKEQQKARDGREGSRLWTEGEKQQLLSTGRVQGYEGYYVLPVEQYPELADSSS
NIQFLRQNEMGKR

| | | |
|---|---|---|
| NOV7t, CG50426-13 | SEQ ID NO: 151 | 1605 bp |
| DNA Sequence | ORF Start: at 405 | ORF Stop: at 1605 |

AGCTGAGGTAGCCGGCTCTGGCCTCGGCTAGCCCAGAAAGGGGCTCCCACAGTGCAGCAGCGGGCTGA
AGGGCTCCTCAAGCGTGGCCAGAGTGGGCGCTGAGGCCGAGGAGGCACCGAGAGCGAGCGAGGGCTGC
AAGGTCTGCCAGCACGCTGTCACTTCTCACTATTTTGATTGGTATTGTGTCCGGAATTGGTGGGTTCT
TGGTCTCACTGACTTCAAGAATGAAGCCACGGACCCTCGCGTTACATCCAGCTTTTTAATTGCCTAAG
CGGTCTGGGGCTTGCTTCGTCATTTGGCTTTGCTGTGGAGCACTCCTGTAAAGCCAGCTGAATTGTAC
ATCGAAGATCCACCCTTTTGGTGGCAAAGTGACAAGTGCCAAAACTCAGGCCTGACTTTTCTGAAAAC
ATCAGCATTCTGCCATATCTGGAATAATGGATGTAAAGGACCGGCGACACCGCTCTTTGACCAGAGGA
CGCTGTGGCAAAGAGTGTCGCTACACAAGCTCCTCTCTGGACAGTGAGGACTGCCGGGTGCCCACACA
GAAATCCTACAGCTCCAGTGAGACTCTGAAGGCCTATGACCATGACAGCAGGATGCACTATGGAAACC
GAGTCACAGACCTCATCCACCGGGAGTCAGATGAGTTTCCTAGACAAGGAACCAACTTCACCCTTGCC
GAACTGGGCATCTGTGAGCCCTCCCCACACCGAAGCGGCTACTGCTCCGACATGGGGATCCTTCACCA
GGGCTACTCCCTTAGCACAGGGTCTGACGCCGACTCCGACACCGAGGGAGGGATGTCTCCAGAACACG
CCATCAGACTGTGGGGCAGAGGGATAAAATCCAGGCGCAGTTCCGGCCTGTCCAGTCGTGAAAACTCG
GCCCTTACCCTGACTGACTCTGACAACGAAAACAAATCAGATGATGAGAACGGTCGTCCCATTCCACC
TACATCCTCGCCTAGTCTCCTCCCATCTGCTCAGCTGCCTAGCTCCCATAATCCTCCACCAGTTAGCT
GCCAGATGCCATTGCTAGACAGCAACACCTCCCATCAAATCATGGACACCAACCCTGATGAGGAATTC
TCCCCCAATTCATACCTGCTCAGAGCATGCTCAGGGCCCCAGCAAGCCTCCAGCAGTGGCCCTCCGAA
CCACCACAGCCAGTCGACTCTGAGGCCCCCTCTCCCACCCCCTCACAACCACACGCTGTCCCATCACC
ACTCGTCCGCCAACTCCCTCAACAGGAACTCACTGACCAATCGGCGGAGTCAGATCCACGCCCCGGCC
CCAGCGCCCAATGACCTGGCCACCACACCAGAGTCCGTTCAGCTTCAGGACAGCTGGGTGCTAAACAG
CAACGTGCCACTGGAGACCCGGCACTTCCTCTTCAAGACCTCCTCGGGGAGCACACCCTTGTTCAGCA
GCTCTTCCCCGGGATACCCTTTGACCTCAGGAACGGTTTACACGCCCCCGCCCCGCCTGCTGCCCAGG
AATACTTTCTCCAGGAAGGCTTTCAAGCTGAAGAAGCCCTCCAAATACTGCAGCTGGAAATGTGCTGC
CCTCTCCGCCATTGCCGCGGCCCTCCTCTTGGCTATTTTGC

| | | | |
|---|---|---|---|
| NOV7t, CG50426-13 | SEQ ID NO: 152 | 400 aa | MW at 43764.8kD |
| Protein Sequence | | | |

KHQHSAISGIMDVKDRRHRSLTRGRCGKECRYTSSSLDSEDCRVPTQKSYSSSETLKAYDHDSRMHYG
NRVTDLIHRESDEFPRQGTNFTLAELGICEPSPHRSGYCSDMGILHQGYSLSTGSDADSDTEGGMSPE
HAIRLWGRGIKSRRSSGLSSRENSALTLTDSDNENKSDDENGRPIPPTSSPSLLPSAQLPSSHNPPPV
SCQMPLLDSNTSHQIMDTNPDEEFSPNSYLLRACSGPQQASSSGPPNHHSQSTLRPPLPPPHNHTLSH
HHSSANSLNRNSLTNRRSQIHAPAPAPUDLATTPESVQLQDSWVLNSNVPLETRHFLFKTSSGSTPLF
SSSSPGYPLTSGTVYTPPPRLLPRINTFSPAFKLKKPSKYCSWKCAALSAIAAALLLAIL

| | | |
|---|---|---|
| NOV7u, CG50426-14 | SEQ ID NO: 153 | 9823 bp |
| DNA Sequence | ORF Start: ATG at 277 | ORF Stop: TAA at 8476 |

GCTCCAAAGCGAGCTGGGACCGAAGACTCTAGGCTAAGTTATCTATGTAGATGGTGTCAGGGAGCGAA
GCTACTGACCGAGCTGCTGTTACATCCAGCTTTTTAATTGCCTAAGCGGTCTGGGGCTTGCTTCGTCA
TTTGGCTTTGCTGTGGAGCACTCCTGTAAAGCCAGCTGAATTGTACATCGAAGATCCACCCTTTTGGT
GGCAAAGTGACAAGTGCCAAAACTCAGGCCTGACTTTTCTGAAAACATCAGCATTCTGCCATATCTGG
AATAATGGATGTAAAGGACCGGCGACACCGCTCTTTGACCAGAGGACGCTGTGGCAAAGAGTGTCGCT
ACACAAGCTCCTCTCTGGACAGTGAGGACTGCCGGGTGCCCACACAGAAATCCTACAGCTCCAGTGAG
ACTCTGAAGGCCTATGACCATGACAGCAGGATGCACTATGGAAACCGAGTCACAGACCTCATCCACCG
GGAGTCAGATGAGTTTCCTAGACAAGGAACCAACTTCACCCTTGCCGAACTGGGCATCTGTGAGCCCT
CCCCACACCGAAGCGGCTACTGCTCCGACATGGGGATCCTTCACCAGGGCTACTCCCTTAGCACAGGG
TCTGACGCCGACTCCGACACCGAGGGAGGGATGTCTCCAGAACACGCCATCAGACTGTGGGGCAGAGG
GATAAAATCCAGGCGCAGTTCCGGCCTGTCCAGTCGTGAAAACTCGGCCCTTACCCTGACTGACTCTG
ACAACGAAAACAAATCAGATGATGAGAACGGTCGTCCCATTCCACCTACATCCTCGCCTAGTCTCCTC
CCATCTGCTCAGCTGCCTAGCTCCCATAATCCTCCACCAGTTAGCTGCCAGATGCCATTGCTAGACAG
CAACACCTCCCATCAAATCATGGACACCAACCCTGATGAGGAATTCTCCCCCAATTCATACCTGCTCA
GAGCATGCTCAGGGCCCCAGCAAGCCTCCAGCAGTGGCCCTCCGAACCACCACAGCCAGTCGACTCTG
AGGCCCCCTCTCCCACCCCCTCACAACCACACGCTGTCCCATCACCACTCGTCCGCCAACTCCCTCAA
CAGGAACTCACTGACCAATCGGCGGAGTCAGATCCACGCCCCGGCCCCAGCGCCCAATGACCTGGCCA
CCACACCAGAGTCCGTTCAGCTTCAGGACAGCTGGGTGCTAAACAGCAACGTGCCACTGGAGACCCGG
CACTTCCTCTTCAAGACCTCCTCGGGGAGCACACCCTTGTTCAGCAGCTCTTCCCCGGGATACCCTTT
GACCTCAGGAACGGTTTACACGCCCCCGCCCCGCCTGCTGCCCAGGAATACTTTCTCCAGGAAGGCTT
TCAAGCTGAAGAAGCCCTCCAAATACTGCAGCTGGAAATGTGCTGCCCTCTCCGCCATTGCCGCGGCC
CTCCTCTTGGCTATTTTGCTGGCGTATTTCATAGTGCCCTGGTCGTTGAAAAACAGCAGCATAGACAG
TGGTGAAGCAGAAGTTGGTCGGCGGGTAACACAAGAAGTCCCACCAGGGGTGTTTTGGAGGTCACAAA
TTCACATCAGTCAGCCCCAGTTCTTAAAGTTCAACATCTCCCTCGGGAAGGACGCTCTCTTTGGTGTT
TACATAAGAAGAGGACTTCCACCATCTCATGCCCAGTATGACTTCATGGAACGTCTGGACGGGAAGGA
GAAGTGGAGTGTGGTTGAGTCTCCCAGGGAACGCCGGAGCATACAGACCTTGGTTCAGAATGAAGCCG
TGTTTGTGCAGTACCTGGATGTGGGCCTGTGGCATCTGGCCTTCTACAATGATGGAAAAGACAAAGAG
ATGGTTTCCTTCAATACTGTTGTCCTAGATTCAGTGCAGGACTGTCCACGTAACTGCCATGGGAATGG
TGAATGTGTGTCCGGGGTGTGTCACTGTTTCCCAGGATTTCTAGGAGCAGACTGTGCTAAAGCTGCCT
GCCCTGTCCTGTGCAGTGGGAATGGACAATATTCTAAAGGGACGTGCCAGTGCTACAGCGGCTGGAAA
GGTGCAGAGTGCGACGTGCCCATGAATCAGTGCATCGATCCTTCCTGCGGGGGCCACGGCTCCTGCAT
TGATGGGAACTGTGTCTGCTCTGCTGGCTACAAAGGCGAGCACTGTGAGGAAGTTGATTGCTTGGATC
CCACCTGCTCCAGCCACGGAGTCTGTGTGAATGGAGAATGCCTGTGCAGCCCTGGCTGGGGTGGTCTG
AACTGTGAGCTGGCGAGGGTCCAGTGCCCAGACCAGTGCAGTGGGCATGGCACGTACCTGCCTGACAC
GGGCCTCTGCAGCTGCGATCCCAACTGGATGGGTCCCGACTGCTCTGTTGAAGTGTGCTCAGTAGACT
GTGGCACTCACGGCGTCTGCATCGGGGGAGCCTGCCGCTGTGAAGAGGGCTGGACAGGCGCAGCGTGT
GACCAGCGCGTGTGCCACCCCCGCTGCATTGAGCACGGGACCTGTAAAGATGGCAAATGTGAATGCCG
AGAGGGCTGGAATGGTGAACACTGCACCATTGGTAGGCAAACGGCAGGCACCGAAACAGATGGCTGCC
CTGACTTGTGCAACGGTAACGGGAGATGCACACTGGGTCAGAACAGCTGGCAGTGTGTCTGCCAGACC
GGCTGGAGAGGGCCCGGATGCAACGTTGCCATGGAAACTTCCTGTGCTGATAACAAGGATAATGAGGG
AGATGGCCTGGTGGATTGTTTGGACCCTGACTGCTGCCTGCAGTCAGCCTGTCAGAACAGCCTGCTCT
GCCGGGGGTCCCGGGACCCACTGGACATCATTCAGCAGGGCCAGACGGATTGGCCCGCAGTGAAGTCC
TTCTATGACCGTATCAAGCTCTTGGCAGGCAAGGATAGCACCCACATCATTCCTGGAGAGAACCCTTT
CAACAGCAGCTTGGTTTCTCTCATCCGAGGCCAAGTAGTAACTACAGATGGAACTCCCCTGGTCGGTG
TGAACGTGTCTTTTGTCAAGTACCCAAAATACGGCTACACCATCACCCGCCAGGATGGCACGTTCGAC
CTGATCGCAAATGGAGGTGCTTCCTTGACTCTACACTTTGAGCGAGCCCCGTTCATGAGCCAGGAGCG
CACTGTGTGGCTGCCGTGGAACAGCTTTTACGCCATGGACACCCTGGTGATGAAGACCGAGGAGAACT
CCATCCCCAGCTGTGACCTCAGTGGCTTTGTCCGGCCTGATCCAATCATCATCTCCTCCCCACTGTCC
ACCTTCTTTAGTGCTGCCCCTGGGCAGAATCCCATCGTGCCTGAGACCCAGGTTCTTCATGAAGAAAT
CGAGCTCCCTGGTTCCAATGTGAAACTTCGCTATCTGAGCTCTAGAACTGCAGGGTACAAGTCACTGC
TGAAGATCACCATGACCCAGTCCACAGTGCCCCTGAACCTCATTAGGGTTCACCTGATGGTGGCTGTC
GAGGGGCATCTCTTCCAGAAGTCATTCCAGGCTTCTCCCAACCTGGCCTCCACCTTCATCTGGGACAA
GACAGATGCGTATGGCCAAAGGGTGTATGGACTCTCAGATGCTGTTGTGTCTGTCGGGTTTGAATATG
AGACCTGTCCCAGTCTAATTCTCTGGGAGAAAAGGACAGCCCTCCTTCAGGGATTCGAGCTGGACCCC
TCCAACCTCGGTGGCTGGTCCCTAGACAAACACCACATCCTCAATGTTAAAAGTGGAATCCTACACAA
AGGCACTGGGGAAAACCAGTTCCTGACCCAGCAGCCTGCCATCATCACCAGCATCATGGGCAATGGTC
GCCGCCGGAGCATTTCCTGTCCCAGCTGCAACGGCCTTGCTGAAGGCAACAAGCTGCTGGCCCCAGTG
GCTCTGGCTGTTGGAATCGATGGGAGCCTCTATGTGGGTGACTTCAATTACATCCGACGCATCTTTCC
CTCTCGAAATGTGACCAGCATCTTGGAGTTACGAAATAAAGAGTTTAAACATAGCAACAACCCAGCAC
ACAAGTACTACTTGGCAGTGGACCCCGTGTCCGGCTCGCTCTACGTGTCCGACACCAACAGCAGGAGA
ATCTACCGCGTCAAGTCTCTGAGTGGAACCAAAGACCTGGCTGGGAATTCGGAAGTTGTGGCAGGGAC
GGGAGAGCAGTGTCTACCCTTTGATGAAGCCCGCTGCGGGGATGGAGGGAAGGCCATAGATGCAACCC
TGATGAGCCCGAGAGGTATTGCAGTAGACAAGAATGGGCTCATGTACTTTGTCGATGCCACCATGATC
CGGAAGGTTGACCAGAATGGAATCATCTCCACCCTGCTGGGCTCCAATGACCTCACTGCCGTCCGGCC
GCTGAGCTGTGATTCCAGCATGGATGTAGCCCAGGTTCGTCTGGAGTGGCCAACAGACCTTGCTGTCA
ATCCCATGGATAACTCCTTGTATGTTCTAGAGAACAATGTCATCCTTCGAATCACCGAGAACCACCAA
GTCAGCATCATTGCGGGACGCCCCATGCACTGCCAAGTTCCTGGCATTGACTACTCACTCAGCAAACT
AGCCATTCACTCTGCCCTGGAGTCAGCCAGTGCCATTGCCATTTCTCACACTGGGGTCCTCTACATCA
CTGAGACAGATGAGAAGAAGATTAACCGTCTACGCCAGGTAACAACCAACGGGGAGATCTGCCTTTTA
GCTGGGGCAGCCTCGGACTGCGACTGCAAAAACGATGTCAATTGCAACTGCTATTCAGGAGATGATGC
CTACGCGACTGATGCCATCTTGAATTCCCCATCATCCTTAGCTGTAGCTCCAGATGGTACCATTTACA
TTGCAGACCTTGGAAATATTCGGATCAGGGCGGTCAGCAAGAACAAGCCTGTTCTTAATGCCTTCAAC
CAGTATGAGGCTGCATCCCCCGGAGAGCAGGAGTTATATGTTTTCAACGCTGATGGCATCCACCAATA
CACTGTGAGCCTGGTGACAGGGGAGTACTTGTACAATTTCACATATAGTACTGACAATGATGTCACTG
AATTGATTGACAATAATGGGAATTCCCTGAAGATCCGTCGGGACAGCAGTGGCATGCCCCGTCACCTG
CTCATGCCTGACAACCAGATCATCACCCTCACCGTGGGCACCAATGGAGGCCTCAAAGTCGTGTCCAC
ACAGAACCTGGAGCTTGGTCTCATGACCTATGATGGCAACACTGGGCTCCTGGCCACCAAGAGCGATG
AAACAGGATGGACGACTTTCTATGACTATGACCACGAAGGCCGCCTGACCAACGTGACGCGCCCCACG
GGGGTGGTAACCAGTCTGCACCGGGAAATGGAGAAATCTATTACCATTGACATTGAGAACTCCAACCG
TGATGATGACGTCACTGTCATCACCAACCTCTCTTCAGTAGAGGCCTCCTACACAGTGGTACAAGATC
AAGTTCGGAACAGCTACCAGCTCTGTAATAATGGTACCCTGAGGGTGATGTATGCTAATGGGATGGGT
ATCAGCTTCCACAGCGAGCCCCATGTCCTAGCGGGCACCATCACCCCCACCATTGGACGCTGCAACAT
CTCCCTGCCTATGGAGAATGGCTTAAACTCCATTGAGTGGCGCCTAAGAAAGGAACAGATTAAAGGCA
AAGTCACCATCTTTGGCAGGAAGCTCCGGGTCCATGGAAGAAATCTCTTGTCCATTGACTATGATCGA
AATATTCGGACTGAAAAGATCTATGATGACCACCGGAAGTTCACCCTGAGGATCATTTATGACCAGGT
GGGCCGCCCCTTCCTCTGGCTGCCCAGCAGCGGGCTGGCAGCTGTCAACGTGTCATACTTCTTCAATG
GGCGCCTGGCTGGGCTTCAGCGTGGGGCCATGAGCGAGAGGACAGACATCGACAAGCAAGGCCGCATC
GTGTCCCGCATGTTCGCTGACGGGAAAGTGTGGAGCTACTCCTACCTTGACAAGTCCATGGTCCTCCT
GCTTCAGAGCCAACGTCAGTATATATTTGAGTATGACTCCTCTGACCGCCTCCTTGCCGTCACCATGC
CCAGCGTGGCCCGGCACAGCATGTCCACACACACCTCCATCGGCTACATCCGTAATATTTACAACCCG
CCTGAAAGCAATGCTTCGGTCATCTTTGACTACAGTGATGACGGCCGCATCCTGAAGACCTCCTTTTT
GGGCACCGGACGCCAGGTGTTCTACAAGTATGGGAAACTCTCCAAGTTATCAGAGATTGTCTACGACA
GTACCGCCGTCACCTTCGGGTATGACGAGACCACTGGTGTCTTGAAGATGGTCAACCTCCAAAGTGGG
GGCTTCTCCTGCACCATCAGGTACCGGAAGATTGGCCCCCTGGTGGACAAGCAGATCTACAGGTTCTC
CGAGGAAGGCATGGTCAATGCCAGGTTTGACTACACCTATCATGACAACAGCTTCCGCATCGCAAGCA
TCAAGCCCGTCATAAGTGAGACTCCCCTCCCCGTTGACCTCTACCGCTATGATGAGATTTCTGGCAAG
GTGGAACACTTTGGTAAGTTTGGAGTCATCTATTATGACATCAACCAGATCATCACCACTGCCGTGAT
GACCCTCAGCAAACACTTCGACACCCATGGGCGGATCAAGGAGGTCCAGTATGAGATGTTCCGGTCCC
TCATGTACTGGATGACGGTGCAATATGACAGCATGGGCAGGGTGATCAAGAGGGAGCTAAAACTGGGG
CCCTATGCCAATACCACGAAGTACACCTATGACTACGATGGGGACGGGCAGCTCCAGAGCGTGGCCGT
CAATGACCGCCCGACCTGGCGCTACAGCTATGACCTTAATGGGAATCTCCACTTACTGAACCCAGGCA
ACAGTGTGCGCCTCATGCCCTTGCGCTATGACCTCCGGGATCGGATAACCAGACTCGGGGATGTGCAG
TACAAAATTGACGACGATGGCTATCTGTGCCAGAGAGGGTCTGACATCTTCGAATACAATTCCAAGGG
CCTCCTAACAAGAGCCTACAACAAGGCCAGCGGGTGGAGTGTCCAGTACCGCTATGATGGCGTAGGAC
GGCGGGCTTCCTACAAGACCAACCTGGGCCACCACCTGCAGTACTTCTACTCTGACCTCCACAACCCG
ACGCGCATCACCCATGTCTACAATCACTCCAACTCGGAGATTACCTCACTGTACTACGACCTCCAGGG
CCACCTCTTTGCCATGGAGAGCAGCAGTGGGGAGGAGTACTATGTTGCCTCTGATAACACAGGGACTC
CTCTGGCTGTGTTCAGCATCAACGGCCTCATGATCAAACAGCTGCAGTACACGGCCTATGGGGAGATT
TATTATGACTCCAACCCCGACTTCCAGATGGTCATTGGCTTCCATGGGGGACTCTATGACCCCCTGAC
CAAGCTGGTCCACTTCACTCAGCGTGATTATGATGTGCTGGCAGGACGATGGACCTCCCCAGACTATA
CCATGTGGAAAAACGTGGGCAAGGAGCCGGCCCCCTTTAACCTGTATATGTTCAAGAGCAACAATCCT
CTCAGCAGTGAGCTAGATTTGAAGAACTACGTGACAGATGTGAAAAGCTGGCTTGTGATGTTTGGATT
TCAGCTTAGCAACATCATTCCTGGCTTCCCGAGAGCCAAAATGTATTTCGTGCCTCCTCCCTATGAAT
TGTCAGAGAGTCAAGCAAGTGAGAATGGACAGCTCATTACAGGTGTCCAACAGACAACAGAGAGACAT
AACCAGGCCTTCATGGCTCTGGAAGGACAGGTCATTACTAAAAAGCTCCACGCCAGCATCCGAGAGAA
AGCAGGTCACTGGTTTGCCACCACCACGCCCATCATTGGCAAAGGCATCATGTTTGCCATCAAAGAAG
GGCGGGTGACCACGGGCGTGTCCAGCATCGCCAGCGAAGATAGCCGCAAGGTGGCATCTGTGCTGAAC
AACGCCTACTACCTGGACAAGATGCACTACAGCATCGAGGGCAAGGACACCCACTACTTTGTGAAGAT
TGGCTCAGCCGATGGCGACCTGGTCACACTAGGCACCACCATCGGCCGCAAGGTGCTAGAGAGCGGGG
TGAACGTGACCGTGTCCCAGCCCACGCTGCTGGTCAACGGCAGGACTCGAAGGTTCACGAACATTGAG
TTCCAGTACTCCACGCTGCTGCTCAGCATCCGCTATGGCCTCACCCCCGACACCCTGGACGAAGAGAA
GGCCCGCGTCCTGGACCAGGCGAGACAGAGGGCCCTGGGCACGGCCTGGGCCAAGGAGCAGCAGAAAG
CCAGGGACGGGAGAGAGGGGAGCCGCCTGTGGACTGAGGGCGAGAAGCAGCAGCTTCTGAGCACCGGG
CGCGTGCAAGGGTACGAGGGATATTACGTGCTTCCCGTGGAGCAATACCCAGAGCTTGCAGACAGTAG
CAGCAACATCCAGTTTTTAAGACAGAATGAGATGGGAAAGAGGTAACAAAATAATCTGCTGCCATTCC
TTGTCTGAATGGCTCAGCAGGAGTAACTGTTATCTCCTCTCCTAAGGAGATGAAGACCTAACAGGGGC
ACTGCGGCTGGGCTGCTTTAGGAGACCAAGTGGCAAGAAAGCTCACATTTTTTGAGTTCAAATGCTAC
TGTCCAAGCGAGAAGTCCCTCATCCTGAAGTAGACTAAAGCCCGGCTGAAAATTCCGAGGAAAACAAA
ACAAACGAATGAATGAACAGACACACACAATGTTCCAAGTTCCCCTAAAATATGACCCACTTGTTCTG
GGTCTACGCAGAAAAGAGACGCAAAGTGTCCAAAAGGAACAAAAGAACAAAAACGAATAAGCAAAGAA
GAAAACAAACAAAAACAAAACAAAACAAACACACGGACCGATAAACAAAGAAGCGAAGATAAGAAAGA
AGGCCTCATATCCAATTACCTCACTCATTCACATGTGAGCGACACGCAGACATCCGCGAGGGCCAGCG
TCACCAGACCAGCTGCGGGACAAACCACTCAGACTGCTTGTAGGACAAATACTTCTGACATTTTCGTT
TAAGCAAATACAGGTGCATTTAAAACACGACTTTGGGGGTGATTTGTGTGTAGCGCCTGGGGAGGGGG
GATAAAAGAGGAGGAGTGAGCACTGGAAATACTTTTTAAAGAAAAAAAAACATGAGGGAATAAAAGAA
ATTCCTATCAAAAATCAAAGTGAAATAATACCATCCAGCACTTAACTCTCAGGTCCCAACTAAGTCTC
GCCTGAGCTAATTTATTTGAGCGCAGAGTGTAAAATTTAATTCAAAATGGTGGCTATAATCACTACAG
ATAAATTTCATACTCTTTTGTCTTTGGAGATTCCATTGTGGACAGTAATACGCAGTTACAGGGTGTAG
TCTGTTTAGATTCCGTAGTTCGTGGGTATCAGTTTCGGTAGAGGTGCAGCATCGTGACACTTTTGCTA
ACAGGTACCACTTCTGATCACCCTGTACATACATGAGCCGAAAGGCACAATCACTGTTTCAGATTTAA
AATTATTAGTGTGTTTGTTTGGTCCAGAAACTGAGACAATCACATGACAGTCACCACGAGGAGAGAAA
ATTTAAAAAATAAAAATAAAAACAAAAAAAATTTTAAAAATTAAAAAAACAAAAATAAAGTCTAATAA
GAACTTTGGTACAGGAACTTTTTTGTAATATACATGTATGAATTGTTCATCGAGTTTTTATATTAATT
TTAATTTGCTGCTAAGCAAAGACTAGGGACAGGCAAAGATAATTTATGGCAAAGTGTTTAAATTGTTT
ATACATAAATAAAGTCTCTAAAACTCCTGTG

| | | | |
|---|---|---|---|
| NOV7u, CG50426-14 | SEQ ID NO: 154 | 2733 aa | MW at 303423.3kD |
| Protein Sequence | | | |

MDVKDRRHRSLTRGRCGKECRYTSSSLDSEDCRVPTQKSYSSSETLKAYDHDSRMHYGNRVTDLIHRE
SDEFPRQGTNFTLAELGICEPSPHRSGYCSDMGILHQGYSLSTGSDADSDTEGGMSPEHAIRLWGRGI
KSRRSSGLSSRENSALTLTDSDNENKSDDENGRPIPPTSSPSLLPSAQLPSSHNPPPVSCQMPLLDSN
TSHQIMDTNPDEEFSPNSYLLRACSGPQQASSSGPPNHHSQSTLRPPLPPPHNHTLSHHHSSANSLNR
NSLTNRRSQIHAPAPAPNDLATTPESVQLQDSWVLNSNVPLETRHFLFKTSSGSTPLFSSSSPGYPLT
SGTVYTPPPRLLPRNTFSRKAFKLKKPSKYCSWKCAALSAIAAALLLAILLAYFIVPWSLKNSSIDSG
EAEVGRRVTQEVPPGVFWRSQIHISQPQFLKFNISLGKDALFGVYIRRGLPPSHAQYDFMERLDGKEK
WSVVESPRERRSIQTLVQNEAVFVQYLDVGLWHLAFYNDGKDKEMVSFNTVVLDSVQDCPRNCHGNGE
CVSGVCHCFPGFLGADCAKAACPVLCSGNGQYSKGTCQCYSGWKGAECDVPMNQCIDPSCGGHGSCID
GNCVCSAGYKGEHCEEVDCLDPTCSSHGVCVNGECLCSPGWGGLNCELARVQCPDQCSGHGTYLPDTG
LCSCDPNWNGPDCSVEVCSVDCGTHGVCIGGACRCEEGWTGAACDQRVCHPRCIEHGTCKDGKCECRE
GWNGEHCTIGRQTAGTETDGCPDLCNGNGRCTLGQNSWQCVCQTGWRGPGCNVAMETSCADNKDNEGD
GLVDCLDPDCCLQSACQNSLLCRGSRDPLDIIQQGQTDWPAVKSFYDRIKLLAGKDSTHIIPGENPFN
SSLVSLIRGQVVTTDGTPLVGVNVSFVKYPKYGYTITRQDGTFDLIANGGASLTLHFERAPFMSQERT
VWLPWNSFYAMDTLVMKTEENSIPSCDLSGFVRPDPIIISSPLSTFFSAAPGQNPIVPETQVLHEEIE
LPGSNVKLRYLSSRTAGYKSLLKITMTQSTVPLNLIRVHLMVAVEGHLFQKSFQASPNLASTFIWDKT
DAYGQRVYGLSDAVVSVGFEYETCPSLILWEKRTALLQGFELDPSNLGGWSLDKHHILNVKSGILHKG
TGENQFLTQQPAIITSIMGNGRRRSISCPSCNGLAEGNKLLAPVALAVGIDGSLYVGDFNYIRRIFPS
RNVTSILELRNKEFKHSNNPAHKYYLAVDPVSGSLYVSDTNSRRIYRVKSLSGTKDLAGNSEVVAGTG
EQCLPFDEARCGDGGKAIDATLMSPRGIAVDKNGLMYFVDATMIRKVDQNGIISTLLGSNDLTAVRPL
SCDSSMDVAQVRLEWPTDLAVNPMDNSLYVLENNVILRITENHQVSIIAGRPMHCQVPGIDYSLSKLA
IHSALESASAIAISHTGVLYITETDEKKINRLRQVTTNGEICLLAGAASDCDCKNDVNCNCYSGDDAY
ATDAILNSPSSLAVAPDGTIYIADLGNIRIRAVSKNKPVLNAYNQYEAASPGEQELYVFNADGIHQYT
VSLVTGEYLYNFTYSTDNDVTELIDNNGNSLKIRRDSSGMPRHLLMPDNQIITLTVGTNGGLKVVSTQ
NLELGLMTYDGNTGLLATKSDETGWTTFYDYDHEGRITNVTRPTGVVTSLHREMEKSITIDIENSNRD
DDVTVITNLSSVEASYTVVQDQVRNSYQLCNNGTLRVMYANGMGISFHSEPHVLAGTITPTIGRCNIS
LPMENGLNSIEWRLRKEQIKGKVTIFGRKLRVHGRNLLSIDYDRNIRTEKIYDDHRKFTLRIIYDQVG
RPFLWLPSSGLAAVNVSYFFNGRLAGLQRGAMSERTDIDKQGRIVSRMFADGKVWSYSYLDKSMVLLL
QSQRQYIFEYDSSDRLLAVTMPSVARHSMSTHTSIGYIRNIYNPPESNASVIFDYSDDGRILKTSFLG
TGRQVFYKYGKLSKLSEIVYDSTAVTFGYDETTGVLKMVNLQSGGFSCTIRYRKIGPLVDKQIYRFSE
EGMVNARFDYTYHDNSFRIASIKPVISETPLPVDLYRYDEISGKVENFGKFGVTYYDINQIITTAVMT
LSKHFDTHGRIKEVQYEMFRSLMYWMTVQYDSMGRVIKRELKLGPYANTTKYTYDYDGDGQLQSVAVN
DRPTWRYSYDLNGNLHLLNPGNSVRLMPLRYDLRDRITRLGDVQYKIDDDGYLCQRGSDIFEYNSKGL
LTRAYNKASGWSVQYRYDGVGRRASYKTNLGHHLQYFYSDLHNPTRITHVYNHSNSEITSLYYDLQGH
LFAMESSSGEEYYVASDNTGTPLAVFSINGLMIKQLQYTAYGEIYYDSNPDFQMVIGFHGGLYDPLTK
LVHFTQRDYDVLAGRWTSPDYTMWKNVGKEPAPFNLYMFKSNNPLSSELDLKNYVTDVKSWLVMFGFQ
LSNIIPGFPRAKMYFVPPPYELSESQASENGQLITGVQQTTERHNQAFMALEGQVITKKLHASIREKA
GHWFATTTPIIGKGIMFAIKEGRVTTGVSSIASEDSRKVASVLNNAYYLDKMHYSIEGKDTHYFVKIG
SADGDLVTLGTTIGRKVLESGVNVTVSQPTLLVNGRTRPFTNIEFQYSTLLLSIRYGLTPDTLDEEKA
RVLDQARQRALGTAWAKEQQKARDGREGSRLWTEGEKQQLLSTGRVQGYEGYYVLPVEQYPELADSSS
NIQFLRQNEMGKR

| | | |
|---|---|---|
| NOV7v, CG50426-16 | SEQ ID NO: 155 | 2478 bp |
| DNA Sequence | ORF Start: at 1 | ORF Stop: end of sequence |

AAAAACAGCAGCATAGACAGTGGTGAAGCAGAAGTTGGTCGGCGGGTAACACAAGAAGTCCCACCAGG
GGTGTTTTGGAGGTCACAAATTCACATCAGTCAGCCCCAGTTCTTAAAGTTCAACATCTCCCTCGGGA
AGGACGCTCTCTTTGGTGTTTACATAAGAAGAGGACTTCCACCATCTCATGCCCAGTATGACTTCATG
GAACGTCTGGACGGGAAGGAGAAGTGGAGTGTGGTTGAGTCTCCCAGGGAACGCCGGAGCATACAGAC
CTTGGTTCAGAATGAAGCCGTGTTTGTGCAGTACCTGGATGTGGGCCTGTGGCATCTGGCCTTCTACA
ATGATGGAAAAGACAAAGAGATGGTTTCCTTCAATACTGTTGTCCTAGATTCAGTGCAGGACTGTCCA
CGTAACTGCCATGGGAATGGTGAATGTGTGTCCGGGGTGTGTCACTGTTTCCCAGGATTTCTAGGAGC
AGACTGTGCTAAAGCTGCCTGCCCTGTCCTGTGCAGTGGGAATGGACAATATTCTAAAGGGACGTGCC
AGTGCTACAGCGGCTGGAAAGGTGCAGAGTGCGACGTGCCCATGAATCAGTGCATCGATCCTTCCTGC
GGGGGCCACGGCTCCTGCATTGATGGGAACTGTGTCTGCTCTGCTGGCTACAAAGGCGAGCACTGTGA
GGAAGTTGATTGCTTGGATCCCACCTGCTCCAGCCACGGAGTCTGTGTGAATGGAGAATGCCTGTGCA
GCCCTGGCTGGGGTGGTCTGAACTGTGAGCTGGCGAGGGTCCAGTGCCCAGACCAGTGCAGTGGGCAT
GGCACGTACCTGCCTGACACGGGCCTCTGCAGCTGCGATCCCAACTGGATGGGTCCCGACTGCTCTGT
TGAAGTGTGCTCAGTAGACTGTGGCACTCACGGCGTCTGCATCGGGGGAGCCTGCCGCTGTGAAGAGG
GCTGGACAGGCGCAGCGTGTGACCAGCGCGTGTGCCACCCCCGCTGCATTGAGCATGGGACCTGTAAA
GATGGCAAATGTGAATGCCGAGAGGGCTGGAATGGTGAACACTGCACCATTGGTAGGCAAACGGCAGG
CACCGAAACAGATGGCTGCCCTGACTTGTGCAACGGTAACGGGAGATGCACACTGGGTCAGAACAGCT
GGCAGTGTGTCTGCCAGACCGGCTGGAGAGGGCCCGGATGCAACGTTGCCATGGAAACTTCCTGTGCT
GATAACAAGGATAATGAGGGAGATGGCCTGGTGGATTGTTTGGACCCTGACTGCTGCCTGCAGTCAGC
CTGTCAGAACAGCCTGCTCTGCCGGGGGTCCCGGGACCCACTGGACATCATTCAGCAGGGCCAGACGG
ATTGGCCCGCAGTGAAGTCCTTCTATGACCGTATCAAGCTCTTGGCAGGCAAGGATAGCACCCACATC
ATTCCTGGAGAGAACCCTTTCAACAGCAGCTTGGTTTCTCTCATCCGAGGCCAAGTAGTAACTACAGA
TGGAACTCCCCTGGTCGGTGTGAACGTGTCTTTTGTCAAGTACCCAAAATACGGCTACACCATCACCC
GCCAGGATGGCACGTTCGACCTGATCGCAAATGGAGGTGCTTCCTTGACTCTACACTTTGAGCGAGCC
CCGTTCATGAGCCAGGAGCGCACTGTGTGGCTGCCGTGGAACAGCTTTTACGCCATGGACACCCTGGT
GATGAAGACCGAGGAGAACTCCATCCCCAGCTGTGACCTCAGTGGCTTTGTCCGGCCTGATCCAATCA
TCATCTCCTCCCCACTGTCCACCTTCTTTAGTGCTGCCCCTGGGCAGAATCCCATCGTGCCTGAGACC
CAGGTTCTTCATGAAGAAATCGAGCTCCCTGGTTCCAATGTGAAACTTCGCTATCTGAGCTCTAGAAC
TGCAGGGTACAAGTCACTGCTGAAGATCACCATGACCCAGTCCACAGTGCCCCTGAACCTCATTAGGG
TTCACCTGATGGTGGCTGTCGAGGGGCATCTCTTCCAGAAGTCATTCCAGGCTTCTCCCAACCTGGCC
TACACCTTCATCTGGGACAAGACAGATGCGTATGGCCAAAGGGTGTATGGACTCTCAGATGCTGTTGT
GTCTGTCGGGTTTGAATATGAGACCTGTCCCAGTCTAATTCTCTGGGAGAAAAGGACAGCCCTCCTTC
AGGGATTCGAGCTGGACCCCTCCAACCTCGGTGGCTGGTCCCTAGACAAACACCACATCCTCAATGTT
AAAAGTGGAATCCTACACAAAGGCACTGGGGAAAACCAGTTCCTGACCCAGCAGCCTGCCATCATCAC
CAGCATCATGGGCAATGGTCGTCGCCGGAGCATTTCTTGTCCCAGCTGCAACGGCCTCGCTGAAGGCA
ACAAGCTGCTGGCCCCAGTGGCTCTGGCTGTTGGAATCGATGGGAGCCTCTATGTGGGTGACTTCAAT
TACATCCGACGCATCTTTCCCTCTCGAAAT

| | | | |
|---|---|---|---|
| NOV7v, CG50426-16 | SEQ ID NO: 156 | 826 aa | MW at 89909.7kD |
| Protein Sequence | | | |

KNSSIDSGEAEVGRRVTQEVPPGVFWRSQIHISQPQFLKFNISLGKDALFGVYIRRGLPPSHAQYDFM
ERLDGKEKWSVVESPRERRSIQTLVQNEAVFVQYLDVGLWHLAFYNDGKDKEMVSFNTVVLDSVQDCP
RNCHGNGECVSGVCHCFPGFLGADCAKAACPVLCSGNGQYSKGTCQCYSGWKGAECDVPNNQCIDPSC
GGHGSCIDGNCVCSAGYKGEHCEEVDCLDPTCSSHGVCVNGECLCSPGWGGLNCELARVQCPDQCSGH
GTYLPDTGLCSCDPNWMGPDCSVEVCSVDCGTHGVCIGGACRCEEGWTGAACDQRVCHPRCIEHGTCK
DGKCECREGWNGEHCTIGRQTAGTETDGCPDLCNGNGRCTLGQNSWQCVCQTGWRGPGCNVAMETSCA
DNKDNEGDGLVDCLDPDCCLQSACQNSLLCRGSRDPLDIIQQGQTDWPAVKSFYDRIKLLAGKDSTHI
IPGENPFNSSLVSLIRGQVVTTDGTPLVGVNVSFVKYPKYGYTITRQDGTFDLIANGGASLTLHFERA
PFMSQERTVWLPWNSFYAMDTLVMKTEENSIPSCDLSGFVRPDPIIISSPLSTFFSAAPGQNPIVPET
QVLHEEIELPGSNVKLRYLSSRTAGYKSLLKITMTQSTVPLNLIRVHLMVAVEGHLFQKSFQASPNLA
YTFIWDKTDAYGQRVYGLSDAVVSVGFEYETCPSLILWEKRTALLQGFELDPSNLGGWSLDKHHILNV
KSGILHKGTGENQFLTQQPAIITSIMGNGRRRSISCPSCNGLAEGNKLLAPVALAVGIDGSLYVGDFN
YIRRIFPSRN

| | | |
|---|---|---|
| NOV7w, CG50426-18 | SEQ ID NO: 157 | 57 bp |
| DNA Sequence | ORF Start: at 1 | ORF Stop: end of sequence |

GACACGGGCCTCTGCAGCTGCGATCCCAACTGGATGGGTCCCGACTGCTCTGTTGAA

| | | | |
|---|---|---|---|
| NOV7w, CG50426-18 | SEQ ID NO: 158 | 19 aa | MW at 2029.2kD |
| Protein Sequence | | | |

DTGLCSCDPNWMGPDCSVE

| | | |
|---|---|---|
| NOV7x, CG50426-19 | SEQ ID NO: 159 | 54 bp |
| DNA Sequence | ORF Start: at 1 | ORF Stop: end of sequence |

GACCGTATCAAGCTCTTGGCAGGCAAGGATAGCACCCACATCATTCCTGGAGAG

| | | | |
|---|---|---|---|
| NOV7x, CG50426-19 | SEQ ID NO: 160 | 18 aa | MW at 1963.2kD |
| Protein Sequence | | | |

DRIKLLAGKDSTHIIPGE

| | | |
|---|---|---|
| NOV7y, CG50426-20 | SEQ ID NO: 161 | 60 bp |
| DNA Sequence | ORF Start: at 1 | ORF Stop: end of sequence |

TCTGCTGGCTACAAAGGCGAGCACTGTGAGGAAGTTGATTGCTTGGATCCCACCTGCTCC

| | | | |
|---|---|---|---|
| NOV7y, CG50426-20 | SEQ ID NO: 162 | 20 aa | MW at 2143.3kD |
| Protein Sequence | | | |

SAGYKGEHCEEVDCLDPTCS

| | | |
|---|---|---|
| NOV7z, CG50426-22 | SEQ ID NO: 163 | 540 bp |
| DNA Sequence | ORF Start: at 1 | ORF Stop: end of sequence |

AACAGCAGCATAGACAGTGGTGAAGCAGAAGTTGGTCGGCGGGTAACACAAGAAGTCCCACCAGGGGT
GTTTTGGAGGTCACAAATTCACATCAGTCAGCCCCAGTTCTTAAAGTTCAACATCTCCCTCGGGAAGG
ACGCTCTCTTTGGTGTTTACATAAGAAGAGGACTTCCACCATCTCATGCCCAGTATGACTTCATGGAA
CGTCTGGACGGGAAGGAGAAGTGGAGTGTGGTTGAGTCTCCCAGGGAACGCCGGAGCATACAGACCTT
GGTTCAGAATGAAGCCGTGTTTGTGCAGTACCTGGATGTGGGCCTGTGGCATCTGGCCTTCTACAATG
ATGGAAAAGACAAAGAGATGGTTTCCTTCAATACTGTTGTCCTAGATGATTCAGTGCAGGACTGTCCA
CGTAACTGCCATGGGAATGGTGAATGTGTGTCCGGGGTGTGTCACTGTTTCCCAGGATTTCTAGGAGC
AGACTGTGCTAAAGACCTTCCTGCCTTGACTTTCTGCAAGACAATCATTAATAAAGCTGCTCTG

| | | | |
|---|---|---|---|
| NOV7z, CG50426-22 | SEQ ID NO: 164 | 180 aa | MW at 20167.7kD |
| Protein Sequence | | | |

NSSIDSGEAEVGRRVTQEVPPGVFWRSQIHISQPQFLKFNISLGKDALFGVYIRRGLPPSHAQYDFME
RLDGKEKWSVVESPRERRSIQTLVQNEAVFVQYLDVGLWHLAFYNDGKDKENVSFNTVVLDDSVQDCP
RNCHGNGECVSGVCHCFPGFLGADCAKDLPALTFCKTIINKAAL

| | | |
|---|---|---|
| NOV7aa, CG50426-23 | SEQ ID NO: 165 | 2497 bp |
| DNA Sequence | ORF Start: at 11 | ORF Stop: at 2489 |

CACCAAGCTTAAAAACAGCAGCATAGACAGTGGTGAAGCAGAAGTTGGTCGGCGGGTAACACAAGAAG
TCCCACCAGGGGTGTTTTGGAGGTCACAAATTCACATCAGTCAGCCCCAGTTCTTAAAGTTCAACATC
TCCCTCGGGAAGGACGCTCTCTTTGGTGTTTACATAAGAAGAGGACTTCCACCATCTCATGCCCAGTA
TGACTTCATGGAACGTCTGGACGGGAAGGAGAAGTGGAGTGTGGTTGAGTCTCCCAGGGAACGCCGGA
GCATACAGACCTTGGTTCAGAATGAAGCCGTGTTTGTGCAGTACCTGGATGTGGGCCTGTGGCATCTG
GCCTTCTACAATGATGGAAAAGACAAAGAGATGGTTTCCTTCAATACTGTTGTCCTAGATTCAGTGCA
GGACTGTCCACGTAACTGCCATGGGAATGGTGAATGTGTGTCCGGGGTGTGTCACTGTTTCCCAGGAT
TTCTAGGAGCAGACTGTGCTAAAGCTGCCTGCCCTGTCCTGTGCAGTGGGAATGGACAATATTCTAAA
GGGACGTGCCAGTGCTACAGCGGCTGGAAAGGTGCAGAGTGCGACGTGCCCATGAATCAGTGCATCGA
TCCTTCCTGCGGGGGCCACGGCTCCTGCATTGATGGGAACTGTGTCTGCTCTGCTGGCTACAAAGGCG
AGCACTGTGAGGAAGTTGATTGCTTGGATCCCACCTGCTCCAGCCACGGAGTCTGTGTGAATGGAGAA
TGCCTGTGCAGCCCTGGCTGGGGTGGTCTGAACTGTGAGCTGGCGAGGGTCCAGTGCCCAGACCAGTG
CAGTGGGCATGGCACGTACCTGCCTGACACGGGCCTCTGCAGCTGCGATCCCAACTGGATGGGTCCCG
ACTGCTCTGTTGAAGTGTGCTCAGTAGACTGTGGCACTCACGGCGTCTGCATCGGGGGAGCCTGCCGC
TGTGAAGAGGGCTGGACAGGCGCAGCGTGTGACCAGCGCGTGTGCCACCCCCGCTGCATTGAGCACGG
GACCTGTAAAGATGGCAAATGTGAATGCCGAGAGGGCTGGAATGGTGAACACTGCACCATTGGTAGGC
AAACGGCAGGCACCGAAACAGATGGCTGCCCTGACTTGTGCAACGGTAACGGGAGATGCACACTGGGT
CAGAACAGCTGGCAGTGTGTCTGCCAGACCGGCTGGAGAGGGCCCGGATGCAACGTTGCCATGGAAAC
TTCCTGTGCTGATAACAAGGATAATGAGGGAGATGGCCTGGTGGATTGTTTGGACCCTGACTGCTGCC
TGCAGTCAGCCTGTCAGAACAGCCTGCTCTGCCGGGGGTCCCGGGACCCACTGGACATCATTCAGCAG
GGCCAGACGGATTGGCCCGCAGTGAAGTCCTTCTATGACCGTATCAAGCTCTTGGCAGGCAAGGATAG
CACCCACATCATTCCTGGAGAGAACCCTTTCAACAGCAGCTTGGTTTCTCTCATCCGAGGCCAAGTAG
TAACTACAGATGGAACTCCCCTGGTCGGTGTGAACGTGTCTTTTGTCAAGTACCCAAAATACGGCTAC
ACCATCACCCGCCAGGATGGCACGTTCGACCTGATCGCAAATGGAGGTGCTTCCTTGACTCTACACTT
TGAGCGAGCCCCGTTCATGAGCCAGGAGCGCACTGTGTGGCTGCCGTGGAACAGCTTTTACGCCATGG
ACACCCTGGTGATGAAGACCGAGGAGAACTCCATCCCCAGCTGTGACCTCAGTGGCTTTGTCCGGCCT
GATCCAATCATCATCTCCTCCCCACTGTCCACCTTCTTTAGTGCTGCCCCTGGGCAGAATCCCATCGT
GCCTGAGACCCAGGTTCTTCATGAAGAAATCGAGCTCCCTGGTTCCAATGTGAAACTTCGCTATCTGA
GCTCTAGAACTGCAGGGTACAAGTCACTGCTGAAGATCACCATGACCCAGTCCACAGTGCCCCTGAAC
CTCATTAGGGTTCACCTGATGGTGGCTGTCGAGGGGCATCTCTTCCAGAAGTCATTCCAGGCTTCTCC
CAACCTGGCCTACACCTTCATCTGGGACAAGACAGATGCGTATGGCCAAAGGGTGTATGGACTCTCAG
ATGCTGTTGTGTCTGTCGGGTTTGAATATGAGACCTGTCCCAGTCTAATTCTCTGGGAGAAAAGGACA
GCCCTCCTTCAGGGATTCGAGCTGGACCCCTCCAACCTCGGTGGCTGGTCCCTAGACAAACACCACAT
CCTCAATGTTAAAAGTGGAATCCTACACAAAGGCACTGGGGAAAACCAGTTCCTGACCCAGCAGCCTG
CCATCATCACCAGCATCATGGGCAATGGTCGCCGCCGGAGCATTTCCTGTCCCAGCTGCAACGGCCTT
GCTGAAGGCAACAAGCTGCTGGCCCCAGTGGCTCTGGCTGTTGGAATCGATGGGAGCCTCTATGTGGG
TGACTTCAATTACATCCGACGCATCTTTCCCTCTCGAAATCTCGAGGGC

| | | | |
|---|---|---|---|
| NOV7aa, CG50426-23 | SEQ ID NO: 166 | 826 aa | MW at 89909.7kD |
| Protein Sequence | | | |

KNSSIDSGEAEVGRRVTQEVPPGVFWRSQIHISQPQFLKFNISLGKDALFGVYIRRGLPPSHAQYDFM
ERLDGKEKWSVVESPRERRSIQTLVQNEAVFVQYLDVGLWHLAFYNDGKDKEMVSFNTVVLDSVQDCP
RNCHGNGECVSGVCHCFPGFLGADCAKAACPVLCSGNGQYSKGTCQCYSGWKGAECDVPNNQCIDPSC
GGHGSCIDGNCVCSAGYKGEHCEEVDCLDPTCSSHGVCVNGECLCSPGWGGLNCELARVQCPDQCSGH
GTYLPDTGLCSCDPNWMGPDCSVEVCSVDCGTHGVCIGGACRCEEGWTGAACDQRVCHPRCIEHGTCK
DGKCECREGWNGEHCTIGRQTAGTETDGCPDLCNGNGRCTLGQNSWQCVCQTGWRGPGCNVAMETSCA
DNKDNEGDGLVDCLDPDCCLQSACQNSLLCRGSRDPLDIIQQGQTDWPAVKSFYDRIKLLAGKDSTHI
IPGENPFNSSLVSLIRGQVVTTDGTPLVGVNVSFVKYPKYGYTITRQDGTFDLIANGGASLTLHFERA
PFMSQERTVWLPWNSFYAMDTLVMKTEENSIPSCDLSGFVRPDPIIISSPLSTFFSAAPGQNPIVPET
QVLHEEIELPGSNVKLRYLSSRTAGYKSLLKITMTQSTVPLNLIRVHLMVAVEGHLFQKSFQASPNLA
YTFIWDKTDAYGQRVYGLSDAVVSVGFEYETCPSLILWEKRTALLQGFELDPSNLGGWSLDKHHILNV
KSGILHKGTGENQFLTQQPAIITSIMGNGRRRSISCPSCNGLAEGNKLLAPVALAVGIDGSLYVGDFN
YIRRIFPSRN

| | | |
|---|---|---|
| NOV7ab, CG50426-24 | SEQ ID NO: 167 | 8575 bp |
| DNA Sequence | ORF Start: ATG at 199 | ORF Stop: TAA at 8476 |

TAAAGTACCTGTCATCTTGACAAGTGGCGGAGCGGAGGAGTCAAGGATTATAAATGATCACAGCCAGG
TCCAGCTCGCCCCGTGATTGGGCTCTCCCGCGATCTGCACCGGGGGAAGCGCATGAGAGGCCAATGAG
ACTTGAACCCTGAGCCTAAGTTGTCACCAGCAGGACTGATGTGCACACAGAAGGAATGAAGTATGGAT
GTGAAAGAACGCAGGCCTTACTGCTCCCTGACCAAGAGCAGACGAGAGAAGGAACGGCGCTACACAAA
TTCCTCCGCAGACAATGAGGAGTGCCGGGTACCCACACAGAAGTCCTACAGTTCCAGCGAGACATTGA
AAGCTTTTGATCATGATTCCTCGCGGCTGCTTTACGGCAACAGAGTGAAGGATTTGGTTCACAGAGAA
GCAGACGAGTTCACTAGACAAAGCAGGATGCACTATGGAAACCGAGTCACAGACCTCATCCACCGGGA
GTCAGATGAGTTTCCTAGACAAGGTATCCTTCACCAGGGCTACTCCCTTAGCACAGGGTCTGACGCCG
ACTCCGACACCGAGGGAGGGATGTCTCCAGAACACGCCATCAGACTGTGGGGCAGAGGGATAAAATCC
AGGCGCAGTTCCGGCCTGTCCAGTCGTGAAAACTCGGCCCTTACCCTGACTGACTCTGACAACGAAAA
CAAATCAGATGATGAGAACGGTCGTCCCATTCCACGTACATCCTCGCGTAGTCTCCTCCCATTTGTTC
AGCTGCCTAGCTCCCATAATCCTCCACCAGTTAGCTGCCAGATGCCATTGCTAGACAGCAACACCTCC
CATCAAATCATGGACACCAACCCTGATGAGGAATTCTCCCCCAATTCATACCTGCTCAGAGCATGCTC
AGGGCCCCAGCAAGCCTCCAGCAGTGGTCCTCCGAACCACCACAGCCAGTCGACTCTGAGGCCCCCTC
TCCCACCCCCTCACAACCACACGCTGTCCCATCACCACTCGTCCGCCAACTCCCTCAACAGGAACTCA
CTGACCAATCGGCGGAGTCAGATCCACGCCCCGGCCCCAGCGCCCAATGACCTGGCCACCACACCAGA
GTCCGTTCAGCTTCAGGACAGCTGGGTGCTAAACAGCAACGTGCCACTGGAGACCCGGCACTTCCTCT
TCAAGACCTCCTCGGGGAGCACACCCTTGTTCAGCAGCTCTTCCCCGGGATACCCTTTGACCTCAGGA
ACGGTTTACACGCCCCCGCCCCGCCTGCTGCCCAGGAATACTTTCTCCAGGAAGGCTTTCAAGCTGAA
GAAGCCCTCCAAATACTGCAGCTGGAAATGTGCTGCCCTCTCCGCCATTGCCGCGGCCCTCCTCTTGG
CTATTTTGCTGGCGTATTTCGCAGCAATGCATCTGCTCGGACTCAATTGGCAACTCCAGCCTGCAGAT
GGGCACACCTTTAACAATGGGATAAGGACCGGCTTACCAGGAAACGATGATGTGGCAACAATGCCATC
TGGAGGCAAAGTGCCCTGGTCGTTGAAAAACAGCAGCATAGACAGTGGTGAAGCAGAAGTTGGTCGGC
GGGTAACACAAGAAGTCCCACCAGGGGTGTTTTGGAGGTCACAAATTCACATCAGTCAGCCCCAGTTC
TTAAAGTTCAACATCTCCCTCGGGAAGGACGCTCTCTTTGGTGTTTACATAAGAAGAGGACTTCCACC
ATCTCATGCCCAGTATGACTTCATGGAACGTCTGGACGGGAAGGAGAAGTGGAGTGTGGTTGAGTCTC
CCAGGGAACGCCGGAGCATACAGACCTTGGTTCAGAATGAAGCCGTGTTTGTGCAGTACCTGGATGTG
GGCCTGTGGCATCTGGCCTTCTACAATGATGGAAAAGACAAAGAGATGGTTTCCTTCAATACTGTTGT
CCTAGATTCAGTGCAGGACTGTCCACGTAACTGCCATGGGAATGGTGAATGTGTGTCCGGGGTGTGTC
ACTGTTTCCCAGGATTTCTAGGAGCAGACTGTGCTAAAGCTGCCTGCCCTGTCCTGTGCAGTGGGAAT
GGACAATATTCTAAAGGGACGTGCCAGTGCTACAGCGGCTGGAAAGGTGCAGAGTGCGACGTGCCCAT
GAATCAGTGCATCGATCCTTCCTGCGGGGGCCACGGCTCCTGCATTGATGGGAACTGTGTCTGCTCTG
CTGGCTACAAAGGCGAGCACTGTGAGGAAGTTGATTGCTTGGATCCCACCTGCTCCAGCCACGGAGTC
TGTGTGAATGGAGAATGCCTGTGCAGCCCTGGCTGGGGTGGTCTGAACTGTGAGCTGGCGAGGGTCCA
GTGCCCAGACCAGTGCAGTGGGCATGGCACGTACCTGCCTGACACGGGCCTCTGCAGCTGCGATCCCA
ACTGGATGGGTCCCGACTGCTCTGTTGTGTGCTCAGTAGACTGTGGCACTCACGGCGTCTGCATCGGG
GGAGCCTGCCGCTGTGAAGAGGGCTGGACAGGCGCAGCGTGTGACCAGCGCGTGTGCCACCCCCGCTG
CATTGAGCACGGGACCTGTAAAGATGGCAAATGTGAATGCCGAGAGGGCTGGAATGGTGAACACTGCA
CCATTGATGGCTGCCCTGACTTGTGCAACGGTAACGGGAGATGCACACTGGGTCAGAACAGCTGGCAG
TGTGTCTGCCAGACCGGCTGGAGAGGGCCCGGATGCAACGTTGCCATGGAAACTTCCTGTGCTGATAA
CAAGGATAATGAGGGAGATGGACTCATTGACTGCATGGATCCCGATTGCTGCCTACAGAGTTCCTGCC
AGAATCAGCCCTATTGTCGGGGACTGCCGGATCCTCAGGACATCATTAGCCAAAGCCTTCAATCGCCT
TCTCAGCAAGCTGCCAAATCCTTTTATGATCGAATCAGTTTCCTTATAGGATCTGATAGCACCCATGT
TATACCTGGAGAAAGTCCTTTCAATAGCTTGGTTTCTCTCATCCGAGGCCAAGTAGTAACTACAGATG
GAACTCCCCTGGTCGGTGTGAACGTGTCTTTTGTCAAGTACCCAAAATACGGCTACACCATCACCCGC
CAGGATGGCACGTACTCCCTCTCCAGGTTCGACCTGATCGCAAATGGAGGTGCTTCCTTGACTCTACA
CTTTGAGCGAGCCCCGTTCATGAGCCAGGAGCGCACTGTGTGGCTGCCGTGGAACAGCTTTTACGCCA
TGGACACCCTGGTGATGAAGACCGAGGAGAACTCCATCCCCAGTTGTGACCTCAGTGGTTTTTGTCGG
CTTGATCCCATCATCATCTCCTCCCCTCTGTCCACTTTCTTTAGTGCTGCCCCTGGGCAGAATCCCAT
CGTGCCTGAGACCCAGGTACTTCATGAAGAAATCGAGCTCCCTGGTTCCAATGTGAAACTTCGCTATC
TGAGCTCTAGAACTGCAGGGTACAAGTCACTGCTGAAGATCACCATGACCCAGTCCACAGTGCCCCTG
AACCTCATTAGGGTTCACCTGATGGTGGCTGTCGAGGGGCATCTCTTCCAGAAGTCATTCCAGGCTTC
TCCCAACCTGGCCTACACCTTCATCTGGGACAAGACAGATGCGTATGGCCAAAGGGTGTATGGACTCT
CAGATGCTGTTGGTATGTTTTGGTTTCAAAGGACAGCCCTCCTTCAGGGATTCGAGCTGGACCCCTCC
AACCTCGGTGGCTGGTCCCTAGACAAACACCACATCCTCAATGTTAAAAGTGGTATCCTACACAAAGG
CACTGGGGAAAACCAGTTCCTGACCCAGCAGCCTGCCATCATCACCAGCATCATGGGCAATGGTCGCC
GCCGGAGCATTTCCTGTCCCAGCTGCAACGGCCTTGCTGAAGGCAACAAGCTGCTGGCCCCAGTGGCT
CTGGCTGTTGGAATCGATGGGAGCCTCTATGTGGGTGACTTCAATTACATCCGACGCATCTTTCCCTC
TCGAAATGTGACCAGCATCTTGGAGTTACGGAGAAATAAAGAGTTTAAACATAGCAACAACCCAGCAC
ACAAGTACTACTTGGCAGTGGACCCCGTGTCCGGCTCGCTCTACGTGTCCGACACCAACAGCAGGAGA
ATCTACCGCGTCAAGTCTCTGAGTGGAACCAAAGACCTGGCTGGGAATTCGGAAGTTGTGGCAGGGAC
GGGAGAGCAGTGTCTACCCTTTGATGAAGCCCGCTGCGGGGATGGAGGGAAGGCCATAGATGCAACCC
TGATGAGCCCGAGAGGTATTGCAGTAGACAAGAATGGGCTCATGTACTTTGTCGATGCCACCATGATC
CGGAAGGTTGACCAGAATGGAATCATCTCCACCCTGCTGGGCTCCAATGACCTCACTGCCGTCCGGCC
GCTGAGCTGTGATTCCAGCATGGATGTAGCCCAAGTTCGTCTGGAGTGGCCAACAGACCTTGCTGTCA
ATCCCATGGATAACTCCTTGTATGTTCTAGAGAACAATGTCATCCTTCGAATCACCGAGAACCACCAA
GTCAGCATCATTGCGGGACGCCCCATGCACTGCCAAGTTCCTGGCATTGACTACTCACTCAGCAAACT
AGCCATTCACTCTGCCCTGGAGTCAGCCAGTGCCATTGCCATTTCTCACACTGGGGTCCTCTACATCA
CTGAGACAGATGAGAAGAAGATTAACCGTCTACGCCAGGTAACAACCAACGGGGAGATCTGCCTTTTA
GCTGGGGCAGCCTCGGACTGCGACTGCAAAAACGATGTCAATTGCAACTGCTATTCAGGAGATGATGC
CTACGCGACTGATGCCATCTTGAATTCCCCATCATCCTTAGCTGTAGCTCCAGATGGTACCATTTACA
TTGCAGACCTTGGAAATATTCGGATCAGGGCGGTCAGCAAGAACAAGCCTGTTCTTAATGCCTTCAAC
CAGTATGAGGCTGCATCCCCCGGAGAGCAGGAGTTATATGTTTTCAACGCTGATGGCATCCACCAATA
CACTGTGAGCCTGGTGACAGGGGAGTACTTGTACAATTTCACATATAGTACTGACAATGATGTCACTG
AATTGATTGACAATAATGGGAATTCCCTGAAGATCCGTCGGGACAGCAGTGGCATGCCCCGTCACCTG
CTCATGCCTGACAACCAGATCATCACCCTCACCGTGGGCACCAATGGAGGCCTCAAAGTCGTGTCCAC
ACAGAACCTGGAGCTTGGTCTCATGACCTATGATGGCAACACTGGGCTCCTGGCCACCAAGAGCGATG
AAACAGGATGGACGACTTTCTATAGCTATGACCACGAAGGCCGCCTGACCAACGTGACGCGCCCCACG
GGGGTGGTAACCAGTCTGCACCGGGAAATGGAGAAATCTATTACCATTGACATTGAGAACTCCAACCG
TGATGATGACGTCACTGTCATCACCAACCTCTCTTCAGTAGAGGCCTCCTACACAGTGGTACAAGATC
AAGTTCGGAACAGCTACCAGCTCTGTAATAATGGTACCCTGAGGGTGATGTATGCTAATGGGATGGGT
ATCAGCTTCCACAGCGAGCCCCATGTCCTAGCGGGCACCATCACCCCCACCATTGGACGCTGCAACAT
CTCCCTGCCTATGGAGAATGGCTTPAACTCCATTGAGTGGCGCCTAAGAPAGGAACAGATTAAAGGCA
AAGTCACCATCTTTGGCAGGAAGCTCGAGGTCCATGGAAGAAATCTCTTGTCCATTGACTATGATCGA
AATATTCGGACTGAAAAGATCTATGATGACCACCGGAAGTTCACCCTGAGGATCATTTATGACCAGGT
GGGCCGCCCCTTCCTCTGGCTGCCCAGCAGCGGGCTGGCAGCTGTCAACGTGTCATACTTCTTCAATG
GGCGCCTGGCTGGGCTTCAGCGTGGGGCCATGAGCGAGAGGACAGACATCGACAAGCAAGGCCGCATC
GTGTCCCGCATGTTCGCTGACGGGAAAGTGTGGAGCTACTCCTACCTTGACAAGATGGTCCTCCTGCT
TCAGAGCCAACGTCAGTATATATTTGAGTATGACTCCTCTGACCGCCTCCTTGCCGTCACCATGCCCA
GCGTGGCCCGGCACAGCATGTCCACACACACCTCCATCGGCTACATCCGTAATATTTACAACCCGCCT
GAAAGCAATGCTTCGGTCATCTTTGACTACAGTGATGACGGCCGCATCCTGAAGACCTCCTTTTTGGG
CACCGGACGCCAGGTGTTCTACAAGTATGGGAAACTCTCCAAGTTATCAGAGATTGTCTACGACAGTA
CCGCCGTCACCTTCGGGTATGACGAGACCACTGGTGTCTTGAAGATGGTCAACCTCCAAAGTGGGGGC
TTCTCCTGCACCATCAGGTACCGGAAGATTGGCCCCCTGGTGGACAAGCAGATCTACAGGTTCTCCGA
GGAAGGCATGGTCAATGCCAGGTTTGACTACACCTATCATGACAACAACTTCCGCATCGCAAGCATCA
AGCCCGTCATAAGTGAGACTCCCCTCCCCGTTGACCTCTACCGCTATGATGAGATTTCTGGCAAGGTG
GAACACTTTGGTAAGTTTGGAGTCATCTATTATGACATCAACCAGATCATCACCACTGCCGTGATGAC
CCTCAGCAAACACTTCGACACCCATGGGCGGATCAAGGAGGTCCAGTATGAGATGTTCCGGTCCCTCA
TGTACTGGATGACGGTGCAATATGACAGCATGGGCAGGGTGATCAAGAGGGAGCTAAAACTGGGGCCC
TATGCCAATACCACGAAGTACACCTATGACTACGATGGGGACGGGCAGCTCCAGAGCGTGCCGGCCGT
CAATGACCGCCCGACCTGGCGCTACAGCTATGACCTTAATGGGAATCTCCACTTACTGAACCCAGGCA
ACAGTGTGCGCCTCATGCCCTTGCGCTATGACCTCCGGGATCGGATAACCAGACTCGGGGATGTGCAG
TACAAAATTGACGACGATGGCTATCTGTGCCAGAGAGGGTCTGACATCTTCGAATACAATTCCAAGGG
CCTCCTAACAAGAGCCTACAACAAGGCCAGCGGGTGGAGTGTCCAGTACCGCTATGATGGCGTAGGAC
GGCGGGCTTCCTACAAGACCAACCTGGGCCACCACCTGCAGTACTTCTACTCTGACCTCCACAACCCG
ACGCGCATCACCCATGTCTACAATCACTCCAACTCGGAGATTACCTCACTGTACTACGACCTCCAGGG
CCACCTCTTTGCCATGGAGAGCAGCAGTGGGGAGGAGTACTATGTTGCCTCTGATAACACAGGGACTC
CTCTGGCTGTGTTCAGCATCAACGGCCTCATGATCAAACAGCTGCAGTACACGGCCTATGGGGAGATT
TATTATGACTCCAACCCCGACTTCCAGATGGTCATTGGCTTCCATGGGGGACTCTATGACCCCCTGAC
CAAGCTGGTCCACTTCACTCAGCGTGATTATGATGTGCTGGCAGGACGATGGACCTCCCCAGACTATA
CCATGTGGAAAAACGTGGGCAAGGAGCCGGCCCCCTTTAACCTGTATATGTTCAAGAGCAACAATCCT
CTCAGCAGTGAGCTAGATTTGAAGAACTACGTGACAGATGTGAAAAGCTGGCTTGTGATGTTTGGATT
TCAGCTTAGCAACATCATTCCTGGCTTCCCGAGAGCCAAAATGTATTTCGTGCCTCCTCCCTATGAAT
TGTCAGAGAGTCAAGCAAGTGAGAATGGACAGCTCATCACAGGTGCCCACCAGACAACAGAGAGACAT
AACCAGGCTTTCATGGCTCTGGAAGGACAGGTCATTACTAkAAAGCTCCACGCCAGCATCCGAGAGAA
AGCAGGTCATTGGTTTGCCACCACCACGCCCATCATTGGCAAAGGCATCATGTTTGCCATCAAAGAAG
GGCGGGTGACCACGGGCGTGTCCAGCATCGCCAGCGAAGATAGCCGCAAGGTGGCATCTGTGCTGAAC
AACGCCTACTACCTGGACAAGATGCACTACAGCATCGAGGGCAAGGACACCCACTACTTTGTGAAGAT
TGGCTCAGCCGATGGCGACCTGGTCACACTAGGCACCACCATCGGCCGCAAGGTGCTAGAGAGCGGGG
TGAACGTGACCGTGTCCCAGCCCACGCTGCTGGTCAACGGCAGGACTCGAAGGTTCACGAACATTGAG
TTCCAGTACTCCACGCTGCTGCTCAGCATCCGCTATGGCCTCACCCCCGACACCCTGGACGAAGAGAA
GGCCCGCGTCCTGGACCAGGCGAGACAGAGGGCCCTGGGCACGGCCTGGGCCAAGGAGCAGCAGAAAG
CCAGGGACGGGAGAGAGGGGAGCCGCCTGTGGACTGAGGGCGAGAAGCAGCAGCTTCTGAGCACCGGG
CGCGTGCAAGGGTACGAGGGATATTACGTGCTTCCCGTGGAGCAATACCCAGAGCTTGCAGACAGTAG
CAGCAACATCCAGTTTTTAAGACAGAATGAGATGGGAAAGAGGTAACAAAATAATCTGCTGCCATTCC
TTGTCTGAATGGCTCAGCAGGAGTAACTGTTATCTCCTCTCCTAAGGAGATGAAGACCTAACAGGGGC
ACTGCGG

| | | | |
|---|---|---|---|
| NOV7ab, CG50426-24 | SEQ ID NO: 168 | 2759 aa | MW at 306902.2kD |
| Protein Sequence | | | |

MDVKERRPYCSLTKSRREKERRYTNSSADNEECRVPTQKSYSSSETLKAFDHDSSRLLYGNRVKDLVH
READEFTRQSRMHYGNRVTDLIHRESDEFPRQGILHQGYSLSTGSDADSDTEGGMSPEHAIRLWGRGI
KSRRSSGLSSRENSALTLTDSDNENKSDDENGRPIPRTSSRSLLPFVQLPSSHNPPPVSCQMPLLDSN
TSHQIMDTNPDEEFSPNSYLLRACSGPQQASSSGPPNHHSQSTLRPPLPPPHNHTLSHHHSSANSLNR
NSLTNPRSQIHAPAPAPNDLATTPESVQLQDSWVLNSNVPLETRHFLFKTSSGSTPLFSSSSPGYPLT
SGTVYTPPPRLLPRNTFSRKAFKLKKPSKYCSWKCAALSAIAAALLLAILLAYFAAMHLLGLNWQLQP
ADGHTFNNGIRTGLPGNDDVATMPSGGKVPWSLKNSSIDSGEAEVGRRVTQEVPPGVFWRSQIHISQP
QFLKFNISLGKDALFGVYIRRGLPPSHAQYDFMERLDGKEKWSVVESPRERRSIQTLVQNEAVFVQYL
DVGLWHLAFYNDGKDKEMVSFNTVVLDSVQDCPRNCHGNGECVSGVCHCFPGFLGADCAKAACPVLCS
GNGQYSKGTCQCYSGWKGAECDVPMNQCIDPSCGGHGSCIDGNCVCSAGYKGEHCEEVDCLDPTCSSH
GVCVNGECLCSPGWGGLNCELARVQCPDQCSGHGTYLPDTGLCSCDPNWMGPDCSVVCSVDCGTHGVC
IGGACRCEEGWTGAACDQRVCHPRCIEHGTCKDGKCECREGWNGEHCTIDGCPDLCNGNGRCTLGQNS
WQCVCQTGWRGPGCNVAMETSCADNKDNEGDGLIDCMDPDCCLQSSCQNQPYCRGLPDPQDIISQSLQ
SPSQQAAKSFYDRISFLIGSDSTHVIPGESPFNSLVSLIRGQVVTTDGTPLVGVNVSFVKYPKYGYTI
TRQDGTYSLSRFDLIANGGASLTLHFERAPFMSQERTVWLPWNSFYAMDTLVMKTEENSIPSCDLSGF
CRLDPIIISSPLSTFFSAAPGQNPIVPETQVLHEEIELPGSNVKLRYLSSRTAGYKSLLKITMTQSTV
PLNLIRVHLMVAVEGHLFQKSFQASPNLAYTFIWDKTDAYGQRVYGLSDAVGMFWFQRTALLQGFELD
PSNLGGWSLDKHHILNVKSGILHKGTGENQFLTQQPAIITSIMGNGRRRSISCPSCNGLAEGNKLLAP
VALAVGIDGSLYVGDFNYIRRIFPSRNVTSILELRRNKEFKHSNNPAHKYYLAVDPVSGSLYVSDTNS
RRIYRVKSLSGTKDLAGNSEVVAGTGEQCLPFDEARCGDGGKAIDATLMSPRGIAVDKNGLMYFVDAT
MIRKVDQNGIISTLLGSNDLTAVRPLSCDSSMDVAQVRLEWPTDIAVNPMDNSLYVLENNVILRITEN
HQVSIIAGRPMHCQVPGIDYSLSKLAIHSALESASAIAISHTGVLYITETDEKKINRLRQVTTNGEIC
LLAGAASDCDCKNDVNCNCYSGDDAYATDATLNSPSSLAVAPDGTIYIADLGNIRIRAVSKNKPVLNA
FNQYEAASPGEQELYVFNADGIHQYTVSLVTGEYLYNFTYSTDNDVTELIDNNGNSLKIRRDSSGMPR
HLLMPDNQIITLTVGTNGGLKVVSTQNLELGLMTYDGNTGLLATKSDETGWTTFYSYDHEGRLTNVTR
PTGVVTSLHREMEKSITIDIENSNRDDDVTVITNLSSVEASYTVVQDQVRNSYQLCNNGTLRVMYANG
MGISFHSEPHVLAGTITPTIGRCNISLPMENGLNSIEWRLRKEQIKGKVTIFGRKLEVHGRNLLSIDY
DRNIRTEKIYDDHRKFTLRIIYDQVGRPFLWLPSSGLAAVNVSYFFNGRLAGLQRGAMSERTDIDKQG
RIVSRMFADGKVWSYSYLDKMVLLLQSQRQYIFEYDSSDRLLAVTMPSVARHSMSTHTSIGYIRNIYN
PPESNASVIFDYSDDGRILKTSFLGTGRQVFYKYGKLSKLSEIVYDSTAVTFGYDETTGVLKMVNLQS
GGFSCTIRYRKIGPLVDKQIYRFSEEGMVNARFDYTYHDNSFRIASIKPVISETPLPVDLYRYDEISG
KVEHFGKFGVIYYDINQIITTAVMTLSKHFDTHGRIKEVQYEMFRSLMYWMTVQYDSMGRVIKRELKL
GPYANTTKYTYDYDGDGQLQSVPAVNDRPTWRYSYDLNGNLHLLNPGNSVRLMPLRYDLRDRITRLGD
VQYKIDDDGYLCQRGSDIFEYNSKGLLTRAYNKASGWSVQYRYDGVGRRASYKTNLGHHLQYFYSDLH
NPTRITHVYNHSNSEITSLYYDLQGHLFAMESSSGEEYYVASDNTGTPLAVFSINGLMIKQLQYTAYG
EIYYDSNPDFQMVIGFHGGLYDPLTKLVHFTQRDYDVLAGRWTSPDYTMWKNVGKEPAPFNLYMFKSN
NPLSSELDLKNYVTDVKSWLVMFGFQLSNIIPGFPRAKMYFVPPPYELSESQASENGQLITGAHQTTE
RHNQAFMALEGQVITKKLHASIREKAGHWFATTTPIIGKGIMFAIKEGRVTTGVSSIASEDSRKVASV
LNNAYYLDKMHYSIEGKDTHYFVKIGSADGDLVTLGTTIGRKVLESGVNVTVSQPTLLVNGRTRRFTN
IEFQYSTLLLSIRYGLTPDTLDEEKARVLDQARQRALGTAWAKEQQKARDGREGSRLWTEGEKQQLLS
TGRVQGYEGYYVLPVEQYPELADSSSNIQFLRQNEMGKR

| | | |
|---|---|---|
| NOV7ac, CG50426-25 | SEQ ID NO: 169 | 6999 bp |
| DNA Sequence | ORF Start: at 1 | ORF Stop: end of sequence |

AAAAACAGCAGCATAGACAGTGGTGAAGCAGAAGTTGGTCGGCGGGTAACACAAGAAGTCCCACCAGG
GGTGTTTTGGAGGTCACAAATTCACATCAGTCAGCCCCAGTTCTTAAAGTTCAACATCTCCCTCGGGA
AGGACGCTCTCTTTGGTGTTTACATAAGAAGAGGACTTCCACCATCTCATGCCCAGTATGACTTCATG
GAACGTCTGGACGGGAAGGAGAAGTGGAGTGTGGTTGAGTCTCCCAGGGAACGCCGGAGCATACAGAC
CTTGGTTCAGAATGAAGCCGTGTTTGTGCAGTACCTGGATGTGGGCCTGTGGCATCTGGCCTTCTACA
ATGATGGAAAAGACAAAGAGATGGTTTCCTTCAATACTGTTGTCCTAGATTCAGTGCAGGACTGTCCA
CGTAACTGCCATGGGAATGGTGAATGTGTGTCCGGGGTGTGTCACTGTTTCCCAGGATTTCTAGGAGC
AGACTGTGCTAAAGCTGCCTGCCCTGTCCTGTGCAGTGGGAATGGACAATATTCTAAAGGGACGTGCC
AGTGCTACAGCGGCTGGAAAGGTGCAGAGTGCGACGTGCCCATGAATCAGTGCATCGATCCTTCCTGC
GGGGGCCACGGCTCCTGCATTGATGGGAACTGTGTCTGCTCTGCTGGCTACAAAGGCGAGCACTGTGA
GGAAGTTGATTGCTTGGATCCCACCTGCTCCAGCCACGGAGTCTGTGTGAATGGAGAATGCCTGTGCA
GCCCTGGCTGGGGTGGTCTGAACTGTGAGCTGGCGAGGGTCCAGTGCCCAGACCAGTGCAGTGGGCAT
GGCACGTACCTGCCTGACACGGGCCTCTGCAGCTGCGATCCCAACTGGATGGGTCCCGACTGCTCTGT
TGAAGTGTGCTCAGTAGACTGTGGCACTCACGGCGTCTGCATCGGGGGAGCCTGCCGCTGTGAAGAGG
GCTGGACAGGCGCAGCGTGTGACCAGCGCGTGTGCCACCCCCGCTGCATTGAGCACGGGACCTGTAAA
GATGGCAAATGTGAATGCCGAGAGGGCTGGAATGGTGAACACTGCACCATTGGTAGGCAAACGGCAGG
CACCGAAACAGATGGCTGCCCTGACTTGTGCAACGGTAACGGGAGATGCACACTGGGTCAGAACAGCT
GGCAGTGTGTCTGCCAGACCGGCTGGAGAGGGCCCGGATGCAACGTTGCCATGGAAACTTCCTGTGCT
GATAACAAGGATAATGAGGGAGATGGCCTGGTGGATTGTTTGGACCCTGACTGCTGCCTGCAGTCAGC
CTGTCAGAACAGCCTGCTCTGCCGGGGGTCCCGGGACCCACTGGACATCATTCAGCAGGGCCAGACGG
ATTGGCCCGCAGTGAAGTCCTTCTATGACCGTATCAAGCTCTTGGCAGGCAAGGATAGCACCCACATC
ATTCCTGGAGAGAACCCTTTCAACAGCAGCTTGGTTTCTCTCATCCGAGGCCAAGTAGTAACTACAGA
TGGAACTCCCCTGGTCGGTGTGAACGTGTCTTTTGTCAAGTACCCAAAATACGGCTACACCATCACCC
GCCAGGATGGCACGTTCGACCTGATCGCAAATGGAGGTGCTTCCTTGACTCTACACTTTGAGCGAGCC
CCGTTCATGAGCCAGGAGCGCACTGTGTGGCTGCCGTGGAACAGCTTTTACGCCATGGACACCCTGGT
GATGAAGACCGAGGAGAACTCCATCCCCAGCTGTGACCTCAGTGGCTTTGTCCGGCCTGATCCAATCA
TCATCTCCTCCCCACTGTCCACCTTCTTTAGTGCTGCCCCTGGGCAGAATCCCATCGTGCCTGAGACC
CAGGTTCTTCATGAAGAAATCGAGCTCCCTGGTTCCAATGTGAAACTTCGCTATCTGAGCTCTAGAAC
TGCAGGGTACAAGTCACTGCTGAAGATCACCATGACCCAGTCCACAGTGCCCCTGAACCTCATTAGGG
TTCACCTGATGGTGGCTGTCGAGGGGCATCTCTTCCAGAAGTCATTCCAGGCTTCTCCCAACCTGGCC
GTCTGTCGGGTTTGAATATGAGACCTGTCCCAGTCTAATTCTCTGGGAGAAAAGGACAGCCCTCCTTC
AGGGATTCGAGCTGGACCCCTCCAACCTCGGTGGCTGGTCCCTAGACAAACACCACATCCTCAATGTT
AAAAGTGGAATCCTACACAAAGGCACTGGGGAAAACCAGTTCCTGACCCAGCAGCCTGCCATCATCAC
CAGCATCATGGGCAATGGTCGCCGCCGGAGCATTTCCTGTCCCAGCTGCAACGGCCTTGCTGAAGGCA
ACAAGCTGCTGGCCCCAGTGGCTCTGGCTGTTGGAATCGATGGGAGCCTCTATGTGGGTGACTTCAAT
TACATCCGACGCATCTTTCCCTCTCGAAATGTGACCAGCATCTTGGAGTTACGAAATAAAGAGTTTAA
ACATAGCAACAACCCAGCACACAAGTACTACTTGGCAGTGGACCCCGTGTCCGGCTCGCTCTACGTGT
CCGACACCAACAGCAGGAGAATCTACCGCGTCAAGTCTCTGAGTGGAACCAAAGACCTGGCTGGGAAT
TCGGAAGTTGTGGCAGGGACGGGAGAGCAGTGTCTACCCTTTGATGAAGCCCGCTGCGGGGATGGAGG
GAAGGCCATAGATGCAACCCTGATGAGCCCGAGAGGTATTGCAGTAGACAAGAATGGGCTCATGTACT
TTGTCGATGCCACCATGATCCGGAAGGTTGACCAGAATGGAATCATCTCCACCCTGCTGGGCTCCAAT
GACCTCACTGCCGTCCGGCCGCTGAGCTGTGATTCCAGCATGGATGTAGCCCAGGTTCGTCTGGAGTG
GCCAACAGACCTTGCTGTCAATCCCATGGATAACTCCTTGTATGTTCTAGAGAACAATGTCATCCTTC
GAATCACCGAGAACCACCAAGTCAGCATCATTGCGGGACGCCCCATGCACTGCCAAGTTCCTGGCATT
GACTACTCACTCAGCAAACTAGCCATTCACTCTGCCCTGGAGTCAGCCAGTGCCATTGCCATTTCTCA
CACTGGGGTCCTCTACATCACTGAGACAGATGAGAAGAAGATTAACCGTCTACGCCAGGTAACAACCA
ACGGGGAGATCTGCCTTTTAGCTGGGGCAGCCTCGGACTGCGACTGCAAAAACGATGTCAATTGCAAC
TGCTATTCAGGAGATGATGCCTACGCGACTGATGCCATCTTGAATTCCCCATCATCCTTAGCTGTAGC
TCCAGATGGTACCATTTACATTGCAGACCTTGGAAATATTCGGATCAGGGCGGTCAGCAAGAACAAGC
CTGTTCTTAATGCCTTCAACCAGTATGAGGCTGCATCCCCCGGAGAGCAGGAGTTATATGTTTTCAAC
GCTGATGGCATCCACCAATACACTGTGAGCCTGGTGACAGGGGAGTACTTGTACAATTTCACATATAG
TACTGACAATGATGTCACTGAATTGATTGACAATAATGGGAATTCCCTGAAGATCCGTCGGGACAGCA
GTGGCATGCCCCGTCACCTGCTCATGCCTGACAACCAGATCATCACCCTCACCGTGGGCACCAATGGA
GGCCTCAAAGTCGTGTCCACACAGAACCTGGAGCTTGGTCTCATGACCTATGATGGCAACACTGGGCT
CCTGGCCACCAAGAGCGATGAAACAGGATGGACGACTTTCTATGACTATGACCACGAAGGCCGCCTGA
CCAACGTGACGCGCCCCACGGGGGTGGTAACCAGTCTGCACCGGGAAATGGAGAAATCTATTACCATT
GACATTGAGAACTCCAACCGTGATGATGACGTCACTGTCATCACCAACCTCTCTTCAGTAGAGGCCTC
CTACACAGTGGTACAAGATCAAGTTCGGAACAGCTACCAGCTCTGTAATAATGGTACCCTGAGGGTGA
TGTATGCTAATGGGATGGGTATCAGCTTCCACAGCGAGCCCCATGTCCTAGCGGGCACCATCACCCCC
ACCATTGGACGCTGCAACATCTCCCTGCCTATGGAGAATGGCTTAAACTCCATTGAGTGGCGCCTAAG
AAAGGAACAGATTAAAGGCAAAGTCACCATCTTTGGCAGGAAGCTCCGGGTCCATGGAAGAAATCTCT
TGTCCATTGACTATGATCGAAATATTCGGACTGAAAAGATCTATGATGACCACCGGAAGTTCACCCTG
AGGATCATTTATGACCAGGTGGGCCGCCCCTTCCTCTGGCTGCCCAGCAGCGGGCTGGCAGCTGTCAA
CGTGTCATACTTCTTCAATGGGCGCCTGGCTGGGCTTCAGCGTGGGGCCATGAGCGAGAGGACAGACA
TCGACAAGCAAGGCCGCATCGTGTCCCGCATGTTCGCTGACGGGAAAGTGTGGAGCTACTCCTACCTT
GACAAGTCCATGGTCCTCCTGCTTCAGAGCCAACGTCAGTATATATTTGAGTATGACTCCTCTGACCG
CCTCCTTGCCGTCACCATGCCCAGCGTGGCCCGGCACAGCATGTCCACACACACCTCCATCGGCTACA
TCCGTAATATTTACAACCCGCCTGAAAGCAATGCTTCGGTCATCTTTGACTACAGTGATGACGGCCGC
ATCCTGAAGACCTCCTTTTTGGGCACCGGACGCCAGGTGTTCTACAAGTATGGGAAACTCTCCAAGTT
ATCAGAGATTGTCTACGACAGTACCGCCGTCACCTTCGGGTATGACGAGACCACTGGTGTCTTGAAGA
TGGTCAACCTCCAAAGTGGGGGCTTCTCCTGCACCATCAGGTACCGGAAGATTGGCCCCCTGGTGGAC
AAGCAGATCTACAGGTTCTCCGAGGAAGGCATGGTCAATGCCAGGTTTGACTACACCTATCATGACAA
CAGCTTCCGCATCGCAAGCATCAAGCCCGTCATAAGTGAGACTCCCCTCCCCGTTGACCTCTACCGCT
ATGATGAGATTTCTGGCAAGGTGGAACACTTTGGTAAGTTTGGAGTCATCTATTATGACATCAACCAG
ATCATCACCACTGCCGTGATGACCCTCAGCAAACACTTCGACACCCATGGGCGGATCAAGGAGGTCCA
GTATGAGATGTTCCGGTCCCTCATGTACTGGATGACGGTGCAATATGACAGCATGGGCAGGGTGATCA
AGAGGGAGCTAAAACTGGGGCCCTATGCCAATACCACGAAGTACACCTATGACTACGATGGGGACGGG
CAGCTCCAGAGCGTGGCCGTCAATGACCGCCCGACCTGGCGCTACAGCTATGACCTTAATGGGAATCT
CCACTTACTGAACCCAGGCAACAGTGTGCGCCTCATGCCCTTGCGCTATGACCTCCGGGATCGGATAA
CCAGACTCGGGGATGTGCAGTACAAAATTGACGACGATGGCTATCTGTGCCAGAGAGGGTCTGACATC
TTCGAATACAATTCCAAGGGCCTCCTAACAAGAGCCTACAACAAGGCCAGCGGGTGGAGTGTCCAGTA
CCGCTATGATGGCGTAGGACGGCGGGCTTCCTACAAGACCAACCTGGGCCACCACCTGCAGTACTTCT
ACTCTGACCTCCACAACCCGACGCGCATCACCCATGTCTACAATCACTCCAACTCGGAGATTACCTCA
CTGTACTACGACCTCCAGGGCCACCTCTTTGCCATGGAGAGCAGCAGTGGGGAGGAGTACTATGTTGC
CTCTGATAACACAGGGACTCCTCTGGCTGTGTTCAGCATCAACGGCCTCATGATCAAACAGCTGCAGT
ACACGGCCTATGGGGAGATTTATTATGACTCCAACCCCGACTTCCAGATGGTCATTGGCTTCCATGGG
GGACTCTATGACCCCCTGACCAAGCTGGTCCACTTCACTCAGCGTGATTATGATGTGCTGGCAGGACG
ATGGACCTCCCCAGACTATACCATGTGGAAAAACGTGGGCAAGGAGCCGGCCCCCTTTAACCTGTATA
TGTTCAAGAGCAACAATCCTCTCAGCAGTGAGCTAGATTTGAAGAACTACGTGACAGATGTGAAAAGC
TGGCTTGTGATGTTTGGATTTCAGCTTAGCAACATCATTCCTGGCTTCCCGAGAGCCAAAATGTATTT
CGTGCCTCCTCCCTATGAATTGTCAGAGAGTCAAGCAAGTGAGAATGGACAGCTCATTACAGGTGTCC
AACAGACAACAGAGAGACATAACCAGGCCTTCATGGCTCTGGAAGGACAGGTCATTACTAAAAAGCTC
CACGCCAGCATCCGAGAGAAAGCAGGTCACTGGTTTGCCACCACCACGCCCATCATTGGCAAAGGCAT
CATGTTTGCCATCAAAGAAGGGCGGGTGACCACGGGCGTGTCCAGCATCGCCAGCGAAGATAGCCGCA
AGGTGGCATCTGTGCTGAACAACGCCTACTACCTGGACAAGATGCACTACAGCATCGAGGGCAAGGAC
ACCCACTACTTTGTGAAGATTGGCTCAGCCGATGGCGACCTGGTCACACTAGGCACCACCATCGGCCG
CAAGGTGCTAGAGAGCGGGGTGAACGTGACCGTGTCCCAGCCCACGCTGCTGGTCAACGGCAGGACTC
GAAGGTTCACGAACATTGAGTTCCAGTACTCCACGCTGCTGCTCAGCATCCGCTATGGCCTCACCCCC
GACACCCTGGACGAAGAGAAGGCCCGCGTCCTGGACCAGGCGAGACAGAGGGCCCTGGGCACGGCCTG
GGCCAAGGAGCAGCAGAAAGCCAGGGACGGGAGAGAGGGGAGCCGCCTGTGGACTGAGGGCGAGAAGC
AGCAGCTTCTGAGCACCGGGCGCGTGCAAGGGTACGAGGGATATTACGTGCTTCCCGTGGAGCAATAC
CCAGAGCTTGCAGACAGTAGCAGCAACATCCAGTTTTTAAGACAGAATGAGATGGGAAAGAGG

| | | | |
|---|---|---|---|
| NOV7ac, CG50426-25 | SEQ ID NO: 170 | 2333 aa | MW at 259545.2kD |
| Protein Sequence | | | |

KNSSIDSGEAEVGRRVTQEVPPGVFWRSQIHISQPQFLKFNISLGKDALFGVYIRRGLPPSHAQYDFM
ERLDGKEKWSVVESPRERRSIQTLVQNEAVFVQYLDVGLWHLAFYNDGKDKEMVSFNTVVLDSVQDCP
RNCHGNGECVSGVCHCFPGFLGADCAKAACPVLCSGNGQYSKGTCQCYSGWKGAECDVPMNQCIDPSC
GGHGSCIDGNCVCSAGYKGEHCEEVDCLDPTCSSHGVCVNGECLCSPGWGGLNCELARVQCPDQCSGH
GTYLPDTGLCSCDPNWMGPDCSVEVCSVDCGTHGVCIGGACRCEEGWTGAACDQRVCHPRCIEHGTCK
DGKCECREGWNGEHCTIGRQTAGTETDGCPDLCNGNGRCTLGQNSWQCVCQTGWRGPGCNVAMETSCA
DNKDNEGDGLVDCLDPDCCLQSACQNSLLCRGSRDPLDIIQQGQTDWPAVKSFYDRIKLLAGKDSTHI
IPGENPFNSSLVSLIRGQVVTTDGTPLVGVNVSFVKYPKYGYTITRQDGTFDLIANGGASLTLHFERA
PFMSQERTVWLPWNSFYAMDTLVMKTEENSIPSCDLSGFVRPDPIIISSPLSTFFSAAPGQNPIVPET
QVLHEEIELPGSNVKLRYLSSRTAGYKSLLKITMTQSTVPLNLIRVHLMVAVEGHLFQKSFQASPNLA
STFIWDKTDAYGQRVYGLSDAVVSVGFEYETCPSLILWEKRTALLQGFELDPSNLGGWSLDKHHILNV
KSGILHKGTGENQFLTQQPAIITSIMGNGRRRSISCPSCNGLAEGNKLLAPVALAVGIDGSLYVGDFN
YIRRIFPSRNVTSILELRNKEFKHSNNPAHKYYLAVDPVSGSLYVSDTNSRRIYRVKSLSGTKDLAGN
SEVVAGTGEQCLPFDEARCGDGGKAIDATLMSPRGIAVDKNGLMYFVDATMIRKVDQNGIISTLLGSN
DLTAVRPLSCDSSMDVAQVRLEWPTDLAVNPMDNSLYVLENNVILRITENHQVSIIAGRPMHCQVPGI
DYSLSKLAIHSALESASAIAISHTGVLYITETDEKKINRLRQVTTNGEICLLAGAASDCDCKNDVNCN
CYSGDDAYATDAILNSPSSLAVAPDGTIYIADLGNIRIRAVSKNKPVLNAFNQYEAASPGEQELYVFN
ADGIHQYTVSLVTGEYLYNFTYSTDNDVTELIDNNGNSLKIRRDSSGMPRHLLMPDNQIITLTVGTNG
GLKVVSTQNLELGLMTYDGNTGLLATKSDETGWTTFYDYDHEGRLTNVTRPTGVVTSLHREMEKSITI
DIENSNRDDDVTVITNLSSVEASYTVVQDQVRNSYQLCNNGTLRVMYANGMGISFHSEPHVLAGTITP
TIGRCNISLPMENGLNSIEWRLRKEQIKGKVTIFGRKLRVHGRNLLSIDYDRNIRTEKIYDDHRKFTL
RIIYDQVGRPFLWLPSSGLAAVNVSYFFNGRLAGLQRGAMSERTDIDKQGRIVSRMFADGKVWSYSYL
DKSMVLLLQSQRQYIFEYDSSDRLLAVTMPSVARHSMSTHTSIGYIRNIYNPPESNASVIFDYSDDGR
ILKTSFLGTGRQVFYKYGKLSKLSEIVYDSTAVTFGYDETTGVLKMVNLQSGGFSCTIRYRKIGPLVD
KQIYRFSEEGMVNARFDYTYHDNSFRIASIKPVISETPLPVDLYRYDEISGKVEHFGKFGVIYYDINQ
IITTAVMTLSKHFDTHGRIKEVQYEMFRSLMYWMTVQYDSMGRVIKRELKLGPYANTTKYTYDYDGDG
QLQSVAVNDRPTWRYSYDLNGNLHLLNPGNSVRLMPLRYDLRDRITRLGDVQYKIDDDGYLCQRGSDI
FEYNSKGLLTRAYNKASGWSVQYRYDGVGRPASYKTNLGHHLQYFYSDLHNPTRITHVYNHSNSEITS
LYYDLQGHLFAMESSSGEEYYVASDNTGTPLAVFSINGLMIKQLQYTAYGEIYYDSNPDFQMVIGFHG
GLYDPLTKLVHFTQRDYDVLAGRWTSPDYTMWKNVGKEPAPFNLYMFKSNNPLSSELDLKNYVTDVKS
WLVMFGFQLSNIIPGFPRAKMYFVPPPYELSESQASENGQLITGVQQTTERHNQAFMALEGQVITKKL
HASIREKAGHWFATTTPIIGKGIMFAIKEGRVTTGVSSIASEDSRKVASVLNNAYYLDKMHYSIEGKD
THYFVKIGSADGDLVTLGTTIGRKVLESGVNVTVSQPTLLVNGRTRRFTNIEFQYSTLLLSIRYGLTP
DTLDEEKARVLDQARQRALGTAWAKEQQKARDGREGSRLWTEGEKQQLLSTGRVQGYEGYYVLPVEQY
PELADSSSNIQFLRQNEMGKR

| | | |
|---|---|---|
| NOV7ad, SNP13375665 of | SEQ ID NO: 171 | 2478 bp |
| 1CG50426-15, DNA Sequence | ORF Start: at 1 | ORF Stop: end of sequence |
| | SNP Pos: 799 | SNP Change: G to A |

AAAAACAGCAGCATAGACAGTGGTGAAGCAGAAGTTGGTCGGCGGGTAACACAAGAAGTCCCACCAGG
GGTGTTTTGGAGGTCACAAATTCACATCAGTCAGCCCCAGTTCTTAAAGTTCAACATCTCCCTCGGGA
AGGACGCTCTCTTTGGTGTTTACATAAGAAGAGGACTTCCACCATCTCATGCCCAGTATGACTTCATG
GAACGTCTGGACGGGAAGGAGAAGTGGAGTGTGGTTGAGTCTCCCAGGGAACGCCGGAGCATACAGAC
CTTGGTTCAGAATGAAGCCGTGTTTGTGCAGTACCTGGATGTGGGCCTGTGGCATCTGGCCTTCTACA
ATGATGGAAAAGACAAAGAGATGGTTTCCTTCAATACTGTTGTCCTAGATTCAGTGCAGGACTGTCCA
CGTAACTGCCATGGGAATGGTGAATGTGTGTCCGGGGTGTGTCACTGTTTCCCAGGATTTCTAGGAGC
AGACTGTGCTAAAGCTGCCTGCCCTGTCCTGTGCAGTGGGAATGGACAATATTCTAAAGGGACGTGCC
AGTGCTACAGCGGCTGGAAAGGTGCAGAGTGCGACGTGCCCATGAATCAGTGCATCGATCCTTCCTGC
GGGGGCCACGGCTCCTGCATTGATGGGAACTGTGTCTGCTCTGCTGGCTACAAAGGCGAGCACTGTGA
GGAAGTTGATTGCTTGGATCCCACCTGCTCCAGCCACGGAGTCTGTGTGAATGGAGAATGCCTGTGCA
GCCCTGGCTGGGGTGGTCTGAACTGTGAGCTGGCGAGGGTCCAGTGCCCAAACCAGTGCAGTGGGCAT
GGCACGTACCTGCCTGACACGGGCCTCTGCAGCTGCGATCCCAACTGGATGGGTCCCGACTGCTCTGT
TGAAGTGTGCTCAGTAGACTGTGGCACTCACGGCGTCTGCATCGGGGGAGCCTGCCGCTGTGAAGAGG
GCTGGACAGGCGCAGCGTGTGACCAGCGCGTGTGCCACCCCCGCTGCATTGAGCACGGGACCTGTAAA
GATGGCAAATGTGAATGCCGAGAGGGCTGGAATGGTGAACACTGCACCATTGGTAGGCAAACGGCAGG
CACCGAAACAGATGGCTGCCCTGACTTGTGCAACGGTAACGGGAGATGCACACTGGGTCAGAACAGCT
GGCAGTGTGTCTGCCAGACCGGCTGGAGAGGGCCCGGATGCAACGTTGCCATGGAAACTTCCTGTGCT
GATAACAAGGATAATGAGGGAGATGGCCTGGTGGATTGTTTGGACCCTGACTGCTGCCTGCAGTCAGC
CTGTCAGAACAGCCTGCTCTGCCGGGGGTCCCGGGACCCACTGGACATCATTCAGCAGGGCCAGACGG
ATTGGCCCGCAGTGAAGTCCTTCTATGACCGTATCAAGCTCTTGGCAGGCAAGGATAGCACCCACATC
ATTCCTGGAGAGAACCCTTTCAACAGCAGCTTGGTTTCTCTCATCCGAGGCCAAGTAGTAACTACAGA
TGGAACTCCCCTGGTCGGTGTGAACGTGTCTTTTGTCAAGTACCCAAAATACGGCTACACCATCACCC
GCCAGGATGGCACGTTCGACCTGATCGCAAATGGAGGTGCTTCCTTGACTCTACACTTTGAGCGAGCC
CCGTTCATGAGCCAGGAGCGCACTGTGTGGCTGCCGTGGAACAGCTTTTACGCCATGGACACCCTGGT
GATGAAGACCGAGGAGAACTCCATCCCCAGCTGTGACCTCAGTGGCTTTGTCCGGCCTGATCCAATCA
TCATCTCCTCCCCACTGTCCACCTTCTTTAGTGCTGCCCCTGGGCAGAATCCCATCGTGCCTGAGACC
CAGGTTCTTCATGAAGAAATCGAGCTCCCTGGTTCCAATGTGAAACTTCGCTATCTGAGCTCTAGAAC
TGCAGGGTACAAGTCACTGCTGAAGATCACCATGACCCAGTCCACAGTGCCCCTGAACCTCATTAGGG
TTCACCTGATGGTGGCTGTCGAGGGGCATCTCTTCCAGAAGTCATTCCAGGCTTCTCCCAACCTGGCC
TCCACCTTCATCTGGGACAAGACAGATGCGTATGGCCAAAGGGTGTATGGACTCTCAGATGCTGTTGT
GTCTGTCGGGTTTGAATATGAGACCTGTCCCAGTCTAATTCTCTGGGAGAAAAGGACAGCCCTCCTTC
AGGGATTCGAGCTGGACCCCTCCAACCTCGGTGGCTGGTCCCTAGACAAACACCACATCCTCAATGTT
AAAAGTGGAATCCTACACAAAGGCACTGGGGAAAACCAGTTCCTGACCCAGCAGCCTGCCATCATCAC
CAGCATCATGGGCAATGGTCGCCGCCGGAGCATTTCCTGTCCCAGCTGCAACGGCCTTGCTGAAGGCA
ACAAGCTGCTGGCCCCAGTGGCTCTGGCTGTTGGAATCGATGGGAGCCTCTATGTGGGTGACTTCAAT
TACATCCGACGCATCTTTCCCTCTCGAAAT

| | | | |
|---|---|---|---|
| NOV7ad, SNP13375665 of | SEQ ID NO: 172 | 826 aa | MW at 89832.6kD |
| CG50426-15, Protein | SNP Pos: 267 | SNP Change: Asp to Asn | |
| Sequence | | | |

KNSSIDSGEAEVGRRVTQEVPPGVFWRSQIHISQPQFLKFNISLGKDALFGVYIRRGLPPSHAQYDFM
ERLDGKEKWSVVESPRERRSIQTLVQNEAVFVQYLDVGLWHLAFYNDGKDKEMVSFNTVVLDSVQDCP
RNCHGNGECVSGVCHCFPGFLGADCAKAACPVLCSGNGQYSKGTCQCYSGWKGAECDVPMNQCIDPSC
GGHGSCIDGNCVCSAGYKGEHCEEVDCLDPTCSSHGVCVNGECLCSPGWGGLNCELARVQCPNQCSGH
GTYLPDTGLCSCDPNWMGPDCSVEVCSVDCGTHGVCIGGACRCEEGWTGAACDQRVCHPRCIEHGTCK
DGKCECREGWNGEHCTIGRQTAGTETDGCPDLCNGNGRCTLGQNSWQCVCQTGWRGPGCNVAMETSCA
DNKDNEGDGLVDCLDPDCCLQSACQNSLLCRGSRDPLDIIQQGQTDWPAVKSFYDRIKLLAGKDSTHI
IPGENPFNSSLVSLIRGQVVTTDGTPLVGVNVSFVKYPKYGYTITRQDGTFDLIANGGASLTLHFERA
PFMSQERTVWLPWNSFYAMDTLVMKTEENSIPSCDLSGFVRPDPIIISSPLSTFFSAAPGQNPIVPET
QVLHEEIELPGSNVKLRYLSSRTAGYKSLLKITMTQSTVPLNLIRVHLMVAVEGHLFQKSFQASPNLA
STFIWDKTDAYGQRVYGLSDAVVSVGFEYETCPSLILWEKRTALLQGFELDPSNLGGWSLDKHHILNV
KSGILHKGTGENQFLTQQPAIITSIMGNGRRRSISCPSCNGLAEGNKLLAPVALAVGIDGSLYVGDFN
YIRRIFPSRN

| | | |
|---|---|---|
| NOV7ae, SNP13375213 of | SEQ ID NO: 173 | 2478 bp |
| CG50426-15, DNA Sequence | ORF Start: at 1 | ORF Stop: end of sequence |
| | SNP Pos: 1751 | SNP Change: T to C |

AAAAACAGCAGCATAGACAGTGGTGAAGCAGAAGTTGGTCGGCGGGTAACACAAGAAGTCCCACCAGG
GGTGTTTTGGAGGTCACAAATTCACATCAGTCAGCCCCAGTTCTTAAAGTTCAACATCTCCCTCGGGA
AGGACGCTCTCTTTGGTGTTTACATAAGAAGAGGACTTCCACCATCTCATGCCCAGTATGACTTCATG
GAACGTCTGGACGGGAAGGAGAAGTGGAGTGTGGTTGAGTCTCCCAGGGAACGCCGGAGCATACAGAC
CTTGGTTCAGAATGAAGCCGTGTTTGTGCAGTACCTGGATGTGGGCCTGTGGCATCTGGCCTTCTACA
ATGATGGAAAAGACAAAGAGATGGTTTCCTTCAATACTGTTGTCCTAGATTCAGTGCAGGACTGTCCA
CGTAACTGCCATGGGAATGGTGAATGTGTGTCCGGGGTGTGTCACTGTTTCCCAGGATTTCTAGGAGC
AGACTGTGCTAAAGCTGCCTGCCCTGTCCTGTGCAGTGGGAATGGACAATATTCTAAAGGGACGTGCC
AGTGCTACAGCGGCTGGAAAGGTGCAGAGTGCGACGTGCCCATGAATCAGTGCATCGATCCTTCCTGC
GGGGGCCACGGCTCCTGCATTGATGGGAACTGTGTCTGCTCTGCTGGCTACAAAGGCGAGCACTGTGA
GGAAGTTGATTGCTTGGATCCCACCTGCTCCAGCCACGGAGTCTGTGTGAATGGAGAATGCCTGTGCA
GCCCTGGCTGGGGTGGTCTGAACTGTGAGCTGGCGAGGGTCCAGTGCCCAGACCAGTGCAGTGGGCAT
GGCACGTACCTGCCTGACACGGGCCTCTGCAGCTGCGATCCCAACTGGATGGGTCCCGACTGCTCTGT
TGAAGTGTGCTCAGTAGACTGTGGCACTCACGGCGTCTGCATCGGGGGAGCCTGCCGCTGTGAAGAGG
GCTGGACAGGCGCAGCGTGTGACCAGCGCGTGTGCCACCCCCGCTGCATTGAGCACGGGACCTGTAAA
GATGGCAAATGTGAATGCCGAGAGGGCTGGAATGGTGAACACTGCACCATTGGTAGGCAAACGGCAGG
CACCGAAACAGATGGCTGCCCTGACTTGTGCAACGGTAACGGGAGATGCACACTGGGTCAGAACAGCT
GGCAGTGTGTCTGCCAGACCGGCTGGAGAGGGCCCGGATGCAACGTTGCCATGGAAACTTCCTGTGCT
GATAACAAGGATAATGAGGGAGATGGCCTGGTGGATTGTTTGGACCCTGACTGCTGCCTGCAGTCAGC
CTGTCAGAACAGCCTGCTCTGCCGGGGGTCCCGGGACCCACTGGACATCATTCAGCAGGGCCAGACGG
ATTGGCCCGCAGTGAAGTCCTTCTATGACCGTATCAAGCTCTTGGCAGGCAAGGATAGCACCCACATC
ATTCCTGGAGAGAACCCTTTCAACAGCAGCTTGGTTTCTCTCATCCGAGGCCAAGTAGTAACTACAGA
TGGAACTCCCCTGGTCGGTGTGAACGTGTCTTTTGTCAAGTACCCAAAATACGGCTACACCATCACCC
GCCAGGATGGCACGTTCGACCTGATCGCAAATGGAGGTGCTTCCTTGACTCTACACTTTGAGCGAGCC
CCGTTCATGAGCCAGGAGCGCACTGTGTGGCTGCCGTGGAACAGCTTTTACGCCATGGACACCCTGGT
GATGAAGACCGAGGAGAACTCCATCCCCAGCTGTGACCTCAGTGGCTTTGCCCGGCCTGATCCAATCA
TCATCTCCTCCCCACTGTCCACCTTCTTTAGTGCTGCCCCTGGGCAGAATCCCATCGTGCCTGAGACC
CAGGTTCTTCATGAAGAAATCGAGCTCCCTGGTTCCAATGTGAAACTTCGCTATCTGAGCTCTAGAAC
TGCAGGGTACAAGTCACTGCTGAAGATCACCATGACCCAGTCCACAGTGCCCCTGAACCTCATTAGGG
TTCACCTGATGGTGGCTGTCGAGGGGCATCTCTTCCAGAAGTCATTCCAGGCTTCTCCCAACCTGGCC
TCCACCTTCATCTGGGACAAGACAGATGCGTATGGCCAAAGGGTGTATGGACTCTCAGATGCTGTTGT
GTCTGTCGGGTTTGAATATGAGACCTGTCCCAGTCTAATTCTCTGGGAGAAAAGGACAGCCCTCCTTC
AGGGATTCGAGCTGGACCCCTCCAACCTCGGTGGCTGGTCCCTAGACAAACACCACATCCTCAATGTT
AAAAGTGGAATCCTACACAAAGGCACTGGGGAAAACCAGTTCCTGACCCAGCAGCCTGCCATCATCAC
CAGCATCATGGGCAATGGTCGCCGCCGGAGCATTTCCTGTCCCAGCTGCAACGGCCTTGCTGAAGGCA
ACAAGCTGCTGGCCCCAGTGGCTCTGGCTGTTGGAATCGATGGGAGCCTCTATGTGGGTGACTTCAAT
TACATCCGACGCATCTTTCCCTCTCGAAAT

| | | | |
|---|---|---|---|
| NOV7ae, SNP13375213 of | SEQ ID NO: 174 | 826 aa | MW at 89805.5kD |
| CG50426-15, Protein | SNP Pos: 584 | SNP Change: Val to Ala | |
| Sequence | | | |

KNSSIDSGEAEVGRRVTQEVPPGVFWRSQIHISQPQFLKFNISLGKDALFGVYIRRGLPPSHAQYDFM
ERLDGKEKWSVVESPRERRSIQTLVQNEAVFVQYLDVGLWHLAFYNDGKDKEMVSFNTVVLDSVQDCP
RNCHGNGECVSGVCHCFPGFLGADCAKAACPVLCSGNGQYSKGTCQCYSGWKGAECDVPMNQCIDPSC
GGHGSCIDGNCVCSAGYKGEHCEEVDCLDPTCSSHGVCVNGECLCSPGWGGLNCELARVQCPDQCSGH
GTYLPDTGLCSCDPNWMGPDCSVEVCSVDCGTHGVCIGGACRCEEGWTGAACDQRVCHPRCIEHGTCK
DGKCECREGWNGEHCTIGRQTAGTETDGCPDLCNGNGRCTLGQNSWQCVCQTGWRGPGCNVAMETSCA
DNKDNEGDGLVDCLDPDCCLQSACQNSLLCRGSRDPLDIIQQGQTDWPAVKSFYDRIKLLAGKDSTHI
IPGENPFNSSLVSLIRGQVVTTDGTPLVGVNVSFVKYPKYGYTITRQDGTFDLIANGGASLTLHFERA
PFMSQERTVWLPWNSFYAMDTLVMKTEENSIPSCDLSGFARPDPIIISSPLSTFFSAAPGQNPIVPET
QVLHEEIELPGSNVKLRYLSSRTAGYKSLLKITMTQSTVPLNLIRVHLMVAVEGHLFQKSFQASPNLA
STFIWDKTDAYGQRVYGLSDAVVSVGFEYETCPSLILWEKRTALLQGFELDPSNLGGWSLDKHHILNV
KSGILHKGTGENQFLTQQPAIITSIMGNGRRRSISCPSCNGLAEGNKLLAPVALAVGIDGSLYVGDFN
YIRRIFPSRN

| | | |
|---|---|---|
| NOV7af, SNP13374985 of | SEQ ID NO: 175 | 2478 bp |
| CG50426-15, DNA Sequence | ORF Start: at 1 | ORF Stop: end of sequence |
| | SNP Pos: 2322 | SNP Change: G to A |

AAAAACAGCAGCATAGACAGTGGTGAAGCAGAAGTTGGTCGGCGGGTAACACAAGAAGTCCCACCAGG
GGTGTTTTGGAGGTCACAAATTCACATCAGTCAGCCCCAGTTCTTAAAGTTCAACATCTCCCTCGGGA
AGGACGCTCTCTTTGGTGTTTACATAAGAAGAGGACTTCCACCATCTCATGCCCAGTATGACTTCATG
GAACGTCTGGACGGGAAGGAGAAGTGGAGTGTGGTTGAGTCTCCCAGGGAACGCCGGAGCATACAGAC
CTTGGTTCAGAATGAAGCCGTGTTTGTGCAGTACCTGGATGTGGGCCTGTGGCATCTGGCCTTCTACA
ATGATGGAAAAGACAAAGAGATGGTTTCCTTCAATACTGTTGTCCTAGATTCAGTGCAGGACTGTCCA
CGTAACTGCCATGGGAATGGTGAATGTGTGTCCGGGGTGTGTCACTGTTTCCCAGGATTTCTAGGAGC
AGACTGTGCTAAAGCTGCCTGCCCTGTCCTGTGCAGTGGGAATGGACAATATTCTAAAGGGACGTGCC
AGTGCTACAGCGGCTGGAAAGGTGCAGAGTGCGACGTGCCCATGAATCAGTGCATCGATCCTTCCTGC
GGGGGCCACGGCTCCTGCATTGATGGGAACTGTGTCTGCTCTGCTGGCTACAAAGGCGAGCACTGTGA
GGAAGTTGATTGCTTGGATCCCACCTGCTCCAGCCACGGAGTCTGTGTGAATGGAGAATGCCTGTGCA
GCCCTGGCTGGGGTGGTCTGAACTGTGAGCTGGCGAGGGTCCAGTGCCCAGACCAGTGCAGTGGGCAT
GGCACGTACCTGCCTGACACGGGCCTCTGCAGCTGCGATCCCAACTGGATGGGTCCCGACTGCTCTGT
TGAAGTGTGCTCAGTAGACTGTGGCACTCACGGCGTCTGCATCGGGGGAGCCTGCCGCTGTGAAGAGG
GCTGGACAGGCGCAGCGTGTGACCAGCGCGTGTGCCACCCCCGCTGCATTGAGCACGGGACCTGTAAA
GATGGCAAATGTGAATGCCGAGAGGGCTGGAATGGTGAACACTGCACCATTGGTAGGCAAACGGCAGG
CACCGAAACAGATGGCTGCCCTGACTTGTGCAACGGTAACGGGAGATGCACACTGGGTCAGAACAGCT
GGCAGTGTGTCTGCCAGACCGGCTGGAGAGGGCCCGGATGCAACGTTGCCATGGAAACTTCCTGTGCT
GATAACAAGGATAATGAGGGAGATGGCCTGGTGGATTGTTTGGACCCTGACTGCTGCCTGCAGTCAGC
CTGTCAGAACAGCCTGCTCTGCCGGGGGTCCCGGGACCCACTGGACATCATTCAGCAGGGCCAGACGG
ATTGGCCCGCAGTGAAGTCCTTCTATGACCGTATCAAGCTCTTGGCAGGCAAGGATAGCACCCACATC
ATTCCTGGAGAGAACCCTTTCAACAGCAGCTTGGTTTCTCTCATCCGAGGCCAAGTAGTAACTACAGA
TGGAACTCCCCTGGTCGGTGTGAACGTGTCTTTTGTCAAGTACCCAAAATACGGCTACACCATCACCC
GCCAGGATGGCACGTTCGACCTGATCGCAAATGGAGGTGCTTCCTTGACTCTACACTTTGAGCGAGCC
CCGTTCATGAGCCAGGAGCGCACTGTGTGGCTGCCGTGGAACAGCTTTTACGCCATGGACACCCTGGT
GATGAAGACCGAGGAGAACTCCATCCCCAGCTGTGACCTCAGTGGCTTTGTCCGGCCTGATCCAATCA
TCATCTCCTCCCCACTGTCCACCTTCTTTAGTGCTGCCCCTGGGCAGAATCCCATCGTGCCTGAGACC
CAGGTTCTTCATGAAGAAATCGAGCTCCCTGGTTCCAATGTGAAACTTCGCTATCTGAGCTCTAGAAC
TGCAGGGTACAAGTCACTGCTGAAGATCACCATGACCCAGTCCACAGTGCCCCTGAACCTCATTAGGG
TTCACCTGATGGTGGCTGTCGAGGGGCATCTCTTCCAGAAGTCATTCCAGGCTTCTCCCAACCTGGCC
TCCACCTTCATCTGGGACAGACAGATGCGTATGGCCAAAAGGGTGTATGGACTCTCAGATGCTGTTGT
GTCTGTCGGGTTTGAATATGAGACCTGTCCCAGTCTAATTCTCTGGGAGAAAAGGACAGCCCTCCTTC
AGGGATTCGAGCTGGACCCCTCCAACCTCGGTGGCTGGTCCCTAGACAAACACCACATCCTCAATGTT
AAAAGTGGAATCCTACACAAAGGCACTGGGGAAAACCAGTTCCTGACCCAGCAGCCTGCCATCATCAC
CAGCATCATAGGCAATGGTCGCCGCCGGAGCATTTCCTGTCCCAGCTGCAACGGCCTTGCTGAAGGCA
ACAAGCTGCTGGCCCCAGTGGCTCTGGCTGTTGGAATCGATGGGAGCCTCTATGTGGGTGACTTCAAT
TACATCCGACGCATCTTTCCCTCTCGAAAT

| | | | |
|---|---|---|---|
| NOV7af, SNP13374985 of | SEQ ID NO: 176 | 826 aa | MW at 89815.5kD |
| CG50426-15, Protein | SNP Pos: 774 | | SNP Change: Met to Ile |
| Sequence | | | |

KNSSIDSGEAEVGRRVTQEVPPGVFWRSQIHISQPQFLKFNISLGKDALFGVYIRRGLPPSHAQYDFM
ERLDGKEKWSVVESPRERRSIQTLVQNEAVFVQYLDVGLWHLAFYNDGKDKEMVSFNTVVLDSVQDCP
RNCHGNGECVSGVCHCFPGFLGADCAKAACPVLCSGNGQYSKGTCQCYSGWKGAECDVPMNQCIDPSC
GGHGSCIDGNCVCSAGYKGEHCEEVDCLDPTCSSHGVCVNGECLCSPGWGGLNCELARVQCPDQCSGH
GTYLPDTGLCSCDPNWMGPDCSVEVCSVDCGTHGVCIGGACRCEEGWTGAACDQRVCHPRCIEHGTCK
DGKCECREGWNGEHCTIGRQTAGTETDGCPDLCNGNGRCTLGQNSWQCVCQTGWRGPGCNVAMETSCA
DNKDNEGDGLVDCLDPDCCLQSACQNSLLCRGSRDPLDIIQQGQTDWPAVKSFYDRIKLLAGKDSTHI
IPGENPFNSSLVSLIRGQVVTTDGTPLVGVNVSFVKYPKYGYTITRQDGTFDLIANGGASLTLHFERA
PFMSQERTVWLPWNSFYAMDTLVMKTEENSIPSCDLSGFVRPDPIIISSPLSTFFSAAPGQNPIVPET
QVLHEEIELPGSNVKLRYLSSRTAGYKSLLKITMTQSTVPLNLIRVHLMVAVEGHLFQKSFQASPNLA
STFIWDKTDAYGQRVYGLSDAVVSVGFEYETCPSLILWEKRTALLQGFELDPSNLGGWSLDKHHILNV
KSGILHKGTGENQFLTQQPAIITSIIGNGRRRSISCPSCNGLAEGNKLLAPVALAVGIDGSLYVGDFN
YIRRIFPSRN

A ClustalW comparison of the above protein sequences yields the following sequence alignment shown in Table 7B.

TABLE 7B
Comparison of the NOV7 protein sequences.

| | |
|---|---|
| NOV7a | ------------------------------------------------------------ |
| NOV7b | ------------------------------------------------------------ |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | ------------------------------------------------------------ |
| NOV7m | ------------------------------------------------------------ |
| NOV7n | ------------------------------------------------------------ |
| NOV7o | ------------------------------------------------------------ |
| NOV7p | ------------------------------------------------------------ |
| NOV7q | MDVKDRRHRSLTRGRCGKECRYTSSSLDSEDCRVPTQKSYSSSETLKAYDHDSRNHYGNR |
| NOV7r | ------------------------------------------------------------ |
| NOV7s | ------------------------------------------------------------ |
| NOV7t | ------------------------------------------------------------ |
| NOV7u | ------------------------------------------------------------ |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | ------------------------------------------------------------ |
| NOV7ac | ------------------------------------------------------------ |
| NOV7a | ------------------------------------------------------------ |
| NOV7b | ------------------------------------------------------------ |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | ------------------------------------------------------------ |
| NOV7m | ------------------------------------------------------------ |
| NOV7n | ------------------------------------------------------------ |
| NOV7O | ------------------------------------------------------------ |
| NOV7p | ------------------------------------------------------------ |
| NOV7q | VTDLIHRESDEFPRQGTNFTTAELGICEPSPHRSGYCSDMGILHQGYSLSTGSDADSDTE |
| NOV7r | ------------------------------------------------------------ |
| NOV7s | ------------------------------------------------------------ |
| NOV7t | ------------------------------------------------------------ |
| NOV7u | ------------------------------------------------------------ |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | ------------------------------------------------------------ |
| NOV7ac | ------------------------------------------------------------ |
| NOV7a | ------------------------------------------------------------ |
| NOV7b | --------------------------MDVKDRRHRSLTRGRCGKECRYTSSSLDSEDCRV |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | ------------------------------------------------------------ |
| NOV7m | ------------------------------------------------------------ |
| NOV7n | ------------------------------------------------------------ |
| NOV7o | ------------------------------------------------------------ |
| NOV7p | ------------------------------------------------------------ |
| NOV7q | GGMSPEHAIRLWGRGIKSRRSSGLSSRENSALTLTDSDNENKSDDENGRPIPPTSSPSLL |
| NOV7r | ------------------------------------------------------------ |
| NOV7s | ------------------------------------------------------------ |
| NOV7t | ------------------------------------------------------------ |
| NOV7u | ------------------------------------------------------------ |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | --------------------------MDVKERRPYCSLTKSRREKERRYTNSSADNEECR |
| NOV7ac | ------------------------------------------------------------ |
| NOV7a | ------------------------------------------------------------ |
| NOV7b | PTQKSYSSSETLKAYDHDSRMHYGNRVTDLIHRESDEFPRQGTNFTLAELGICEPSPHRS |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | -------MDVKDRRHRSLTRGRCGKECRYTSSSLDSEDCRVPTQKSYSSSETLKAYDHDS |
| NOV7m | ------------------------------------------------------------ |
| NOV7n | ------------------------------------------------------------ |
| NOV7o | ------------------------------------------------------------ |
| NOV7p | ------------------------------------------------------------ |
| NOV7q | PSAQLPSSHNPPPVSCQMPLLDSNTSHQIMDTNPDEEFSPNSYLLRACSGPQQASSSGPP |
| NOV7r | -------MDVKDRRHRSLTRGRCGKECRYTSSSLDSEDCRVPTQKSYSSSETLKAYDHDS |
| NOV7s | -------MDVKDRRHRSLTRGRCGKECRYTSSSLDSEDCRVPTQKSYSSSETLKAYDHDS |
| NOV7t | ------------------------------------------------------------ |
| NOV7u | -------MDVKDRRHRSLTRGRCGKECRYTSSSLDSEDCRVPTQKSYSSSETLKAYDHDS |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | VPTQKSYSSSETLKAFDHDSSRLLYGNRVKDLVHREADEFTRQSRMHYGNRVTDLIHRES |
| NOV7ac | ------------------------------------------------------------ |
| NOV7a | ------------------------------------------------------------ |
| NOV7b | GYCSDMGILHQGYSLSTGSDADSDTEGGMSPEHAIRLWGRGIKSRRSSGLSSRENSALTL |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | RMHYGNRVTDLIHRESDEFPRQGTNFTLAELGICEPSPHRSGYCSDMGILHQGYSLSTGS |
| NOV7m | ------------------------------------------------------------ |
| NOV7n | ------------------------------------------------------------ |
| NOV7o | ----MDVKDRRHRSLTRGRCGKECRYTSSSLDSEDCRVPTQKSYSSSETLKAYDHDSRMH |
| NOV7p | ------------------------------------------------------------ |
| NOV7q | NHHSQSTLRPPLPPPHNHTLSHHHSSANSLNRNSLTNRRSQIHAPAPAPNDLATTPESVQ |
| NOV7r | RMHYGNRVTDLIHRESDEFPRQGTNFTLAELGICEPSPHRSGYCSDMGILHQGYSLSTGS |
| NOV7s | RNHYGNRVTDLIHRESDEFPRQGTNFTLAELGICEPSPHRSGYCSDMGILHQGYSLSTGS |
| NOV7t | ------------------------------------------------------------ |
| NOV7u | RMHYGNRVTDLIHRESDEFPRQGTNFTLAELGICEPSPHRSGYCSDMGILHQGYSLSTGS |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | DEFPRQGTLHQGYSLSTGSDADSDTEGGMSPEHAIRLWGRGIKSRRSSGLSSRENSALTL |
| NOV7ac | ------------------------------------------------------------ |
| NOV7a | ------------------MDVKDRRHRSLTRGRCGKECRYTSSSLDSEDCRVPTQKSYSS |
| NOV7b | TDSDNENKSDDENGRPIPPTSSPSLLPSAQLPSSHNPPPVSCQMPLLDSNTSHQIMDTNP |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | DADSDTEGGNSPEHAIRLWGRGIKSRRSSGLSSRENSALTLTDSDNENKSDDENGRPIPP |
| NOV7m | ------------------------------------------------------------ |
| NOV7n | ------------------------------------------------------------ |
| NOV7o | YGNRVTDLIHRESDEFPRQGTNFTLAELGICEPSPHRSGYCSDMGILHQGYSLSTGSDAD |
| NOV7p | ------------------------------------------------------------ |
| NOV7q | LQDSWVLNSNVPLETRHFLFKTSSGSTPLFSSSSPGYPLTSGTVYTPPPRLLPRNTFSRK |
| NOV7r | DADSDTEGGMSPEHAIRLWGRGIKSRRSSGLSSRENSALTLTDSDNENKSDDENGRPIPP |
| NOV7s | DADSDTEGGMSPEHAIRLWGRGIKSRRSSGLSSRENSALTLTDSDNENKSDDENGRPIPP |
| NOV7t | ------------------------------------------------------------ |
| NOV7u | DADSDTEGGMSPEHAIRLWGRGIKSRRSSGLSSRENSALTLTDSDNENKSDDENGRPIPP |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | TDSDNENKSDDENGRPIPRTSSRSLLPFVQLPSSHNPPPVSCQMPLLDSNTSHQIMDTNP |
| NOV7ac | ------------------------------------------------------------ |
| NOV7a | SETLKAYDHDSRMHYGNRVTDLIHRESDEFPRQGTNFTLAELGICEPSPHRSGYCSDMGI |
| NOV7b | DEEFSPNSYLLRACSGPQQASSSGPPNHHSQSTLRPPLPPPHNHTLSHHHSSANSLNPNS |
| NOV7c | ----------------------------------------KNSSIDSGEAEVGRRVTQEV |
| NOV7d | -------------------------------------TKLKNSSIDSGEAEVGRRVTQEV |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | TSSPSLLPSAQLPSSHNPPPVSCQMPLLDSNTSHQIMDTNPDEEFSPNSYLLRACSGPQQ |
| NOV7m | ------------------------------------------------------------ |
| NOV7n | ------------------------------------------------------------ |
| NOV7o | SDTEGGMSPEHAIRLWGRGIKSRRSSGLSSRBNSALTLTDSDNENKSDDENGRPIPPTSS |
| NOV7p | ------------------------------------------------------------ |
| NOV7q | AFKLKKPSKYCSWKCAALSAIAAALLLAILLAYFIVPWSLKNSSIDSGEAEVGRRVTQEV |
| NOV7r | TSSPSLLPSAQLPSSHNPPPVSCQMPLLDSNTSHQIMDTNPDEEFSPNSYLLRACSGPQQ |
| NOV7s | TSSPSLLPSAQLPSSHNPPPVSCQMPLLDSNTSHQIMDTNPDEEFSPNSYLLPACSGPQQ |
| NOV7t | ------------------------------------------------------------ |
| NOV7u | TSSPSLLPSAQLPSSHNPPPVSCQMPLLDSNTSHQIMDTNPDEEFSPNSYLLRACSGPQQ |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | DEEFSPNSYLLEACSGPQQASSSGPPNHHSQSTLRPPLPPPHNHTLSHHHSSANSLNPNS |
| NOV7ac | ------------------------------------------------------------ |
| NOV7a | LHQGYSLSTGSDADSDTEGGMSPEHAIRLWGRGIKSPRSSGLSSRENSALTLTDSDNENK |
| NOV7b | LTNRRSQIHAPAPAPNDLATTPESVQLQDSWVLNSNVPLETRHFLFKTSSGSTPLFSSSS |
| NOV7c | PPGVFWRSQIHISQPQFLKFNISLGKDALFGVYIRRGLPPSHAQYDFMERLDGKEKWSVV |
| NOV7d | PPGVFWRSQIHISQPQFLKFNISLGKDALFGVYIRRGLPPSHAQYDFMERLDGKEKWSVV |
| NOV7e | ------------------------------------------------------------ |
| NOV7f | ------------------------------------------------------------ |
| NOV7g | ------------------------------------------------TKLKNSSIDSGE |
| NOV7h | ------------------------------------------------------------ |
| NOV7i | ------------------------------------------------------------ |
| NOV7l | ------------------------------------------------------------ |
| NOV7k | ------------------------------------------------------------ |
| NOV7l | ASSSGPPNHHSQSTLRPPLPPPHNHTLSHHHSSANSLNRNSLTNRRSQIHAPAPAPNDLA |
| NOV7m | ------------------------------------------------------------ |
| NOV7n | ------------------------------------------------------------ |
| NOV7o | PSLLPSAQLPSSHNPPPVSCQMPLLDSNTSHQIMDTNPDEEFSPNSYLLRACSGPQQASS |
| NOV7p | ------------------------------------------------------------ |
| NOV7q | PPGVFWRSQIHISQPQFLKFNISLGKDALFGVYIRRGLPPSHAQYDFMERLDGKEKWSVV |
| NOV7r | ASSSGPPNHHSQSTLRPPLPPPHNHTLSHHHSSANSLNRNSLTNRRSQIHAPAPAPNDLA |
| NOV7s | ASSSGPPNHHSQSTLRPPLPPPHNHTLSHHHSSANSLNRNSLTNRRSQIHAPAPAPNDLA |
| NOV7t | ------------------------------------------------------------ |
| NOV7u | ASSSGPPNHHSQSTLRPPLPPPHNHTLSHHHSSANSLNRNSLTNRRSQIHAPAPAPNDLA |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | LTNRRSQIHAPAPAPNDLATTPESVQLQDSWVLNSNVPLETRHFLFKTSSGSTPLFSSSS |
| NOV7ac | ------------------------------------------------------------ |
| NOV7a | SDDENGRPIPPTSSPSLLPSAQLPSSHNPPPVSCQMPLLDSNTSHQIMDTNPDEEFSPNS |
| NOV7b | PGYPLTSGTVYTPPPRLLPRNTFSRKAFKLKKPSKYCSWKCAALSAIAAALLLAILLAYF |
| NOV7c | ESPRERRSIQTLVQNEAVFVQYLDVGLWHLAFYNDGKDKEMVSFNTVVLDSVQDCPRNCH |
| NOV7d | ESPRERRSIQTLVQNEAVFVQYLDVGLWHLAFYNDGKDKEMVSFNTVVLDSVQDCPRNCH |
| NOV7e | ------------------------------------------------------------ |
| NOV7f | ------------------------------------------------------------ |
| NOV7g | AEVGRRVTQEVPPGVFWRSQIHISQPQFLKFNISLGKDALFGVYIRRGLPPSHAQYDFME |
| NOV7h | ------------------------------------------------------------ |
| NOV7i | ---------------------------------------------MDVKDRRHRSLTRGR |
| NOV7j | ---------------------------------------------MDVKDRRHRSLTRGR |
| NOV7k | ---------------------------------------------MDVKDRRHRSLTRGR |
| NOV7l | TTPESVQLQDSWVLNSNVPLETRHFLFKTSSGSTPLFSSSSPGYPLTSGTVYTPPPRLLP |
| NOV7m | -----------------------------------KHQHSAISGIMDVKDRRHRSLTRGR |
| NOV7n | -----------------------------------KHQHSAISGIMDVKDRRHRSLTRGR |
| NOV7o | SGPPNHHSQSTLRPPLPPPHNHTLSHHHSSANSLNRNSLTNRRSQIHAPAPAPNDLATTP |
| NOV7p | -----------------------------------KHQHSAISGIMDVKDRRHRSLTRGR |
| NOV7q | ESPRERRSIQTLVQNEAVFVQYLDVGLWHLAFYNDGKDKEMVSFNTVVLDSVQDCPRNCH |
| NOV7r | TTPESVQLQDSWVLNSNVPLETRHFLFKTSSGSTPLFSSSSPGYPLTSGTVYTPPPRLLP |
| NOV7s | TTPESVQLQDSWVLNSNVPLETRHFLFKTSSGSTPLFSSSSPGYPLTSGTVYTPPPRLLP |
| NOV7t | -----------------------------------KHQHSAISGIMDVKDRRHRSLTRGR |
| NOV7u | TTPESVQLQDSWVLNSNVPLETRHFLFKTSSGSTPLFSSSSPGYPLTSGTVYTPPPRLLP |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | PGYPLTSGTVYTPPPRLLPRNTFSRKAFKLKKPSKYCSWKCAALSAIAAALLLAILLAYF |
| NOV7ac | ------------------------------------------------------------ |
| NOV7a | YLLRACSGPQQASSSGPPNHHSQSTLRPPLPPPHNHTLSHHHSSANSLNRNSLTNRRSQI |
| NOV7b | IAMHLLGLNWQLQPADGHTFNNGIRTGLPGNDDVATMPSGGKVPWSLKNSSIDSGEAEVG |
| NOV7c | GNGECVSGVCHCFPGFLGADCAKAACPVLCSGNGQYSKGTCQCYSGWKGAECDVPMNQCI |
| NOV7d | GNGECVSGVCHCFPGFLGADCAKAACPVLCSGNGQYSKGTCQCYSGWKGAECDVPMNQCI |
| NOV7e | --------------------------------------------TKLKNSSIDSGEAEVG |
| NOV7f | ------------------------------------------------------------ |
| NOV7g | RLDGKEKWSVVESPRERRSIQTLVQNEAVFVQYLDVGLWHLAFYNDGKDKEMVSFNTVVL |
| NOV7h | ------------------------------------------------------------ |
| NOV7i | CGKECRYTSSSLDSEDCRVPTQKSYSSSETLKAYDHDSRMHYGNRVTDLIHRESDEFPRQ |
| NOV7j | CGKECRYTSSSLDSEDCRVPTQKSYSSSETLKAYDHDSRMHYGNRVTDLIHRESDEFPRQ |
| NOV7k | CGKECRYTSSSLDSEDCRVPTQKSYSSSETLKAYDHDSRMHYGNRVTDLIHRESDEFPRQ |
| NOV7l | RNTFSRKAFKLKKPSKYCSWKCAALSAIAAALLLAILLAYFIVPWSLKNSSIDSGEAEVG |
| NOV7m | CGKECRYTSSSLDSEDCRVPTQKSYSSSETLKAYDHDSRMHYGNRVTDLIHRESDEFPRQ |
| NOV7n | CGKECRYTSSSLDSEDCRVPTQKSYSSSETLKAYDHDSRMHYGNRVTDLIHRESDEFPRQ |
| NOV7o | ESVQLQDSWVLNSNVPLETRHFLFKTSSGSTPLFSSSSPGYPLTSGTVYTPPPRLLPRNT |
| NOV7p | CGKECRYTSSSLDSEDCRVPTQKSYSSSETLKAYDHDSRMHYGNRVTDLIHRESDEFPRQ |
| NOV7q | GNGECVSGVCHCFPGFLGADCAKAACPVLCSGNGQYSKGTCQCYSGWKGAECDVPMNQCI |
| NOV7r | RNTFSRKAFKLKKPSKYCSWKCAALSAIAAALLLAILLAYFIVPWSLKNSSIDSGEAEVG |
| NOV7s | RNTFSRKAFKLKKPSKYCSWKCAALSAIAAALLLAILLAYFIVPWSLKNSSIDSGEAEVG |
| NOV7t | CGKECRYTSSSLDSEDCRVPTQKSYSSSETLKAYDHDSRMHYGNRVTDLIHRESDEFPRQ |
| NOV7u | RNTFSRKAFKLKKPSKYCSWKCAALSAIAAALLLAILLAYFIVPWSLKNSSIDSGEAEVG |
| NOV7v | -----------------------------------------------KNSSIDSGEAEVG |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | AAMHLLGLNWQLQPADGHTFNNGIRTGLPGNDDVATMPSGGKVPWSLKNSSIDSGEAEVG |
| NOV7ac | -----------------------------------------------KNSSIDSGEAEVG |
| NOV7a | HAPAPAPNDLATTPESVQLQDSWVLNSNVPLETRHFLFKTSSGSTPLFSSSSPGYPLTSG |
| NOV7b | RRVTQEVPPGVFWRSQIHISQPQFLKFNISLGKDALFGVYIRRGLPPSHAQYDFMERLDG |
| NOV7c | DPSCGGHGSCIDGNCVCSAGYKGEHCEEVDCLDPTCSSHGVCVNGECLCSPGWGGLNCEL |
| NOV7d | DPSCGGHGSCIDGNCVCSAGYKGEHCEEVDCLDPTCSSHGVCVNGECLCSPGWGGLNCEL |
| NOV7e | RRVTQEVPPGVFWRSQIHISQPQFLKFNISLGKDALFGVYIRRGLPPSHAQYDFMERLDG |
| NOV7f | -----------------------------------------TKLKNSSIDSGEAEVGRRV |
| NOV7g | DSVQDCPRNCHGNGECVSGVCHCFPGFLGADCAKAACPVLCSGNGQYSKGTCQCYSGWKG |
| NOV7h | ------------------------------------------------------------ |
| NOV7i | GTNFTLAELGICEPSPHRSGYCSDMGILHQGYSLSTGSDADSDTEGGMSPEHAIRLWGRG |
| NOV7j | GTNFTLAELGICEPSPHRSGYCSDMGILHQGYSLSTGSDADSDTEGGMSPEHAIRLWGRG |
| NOV7k | GTNFTLAELGICEPSPHRSGYCSDMGILHQGYSLSTGSDADSDTEGGMSPEHAIRLWGRG |
| NOV7l | RRVTQEVPPGVFWRSQIHISQPQFLKFNISLGKDALFGVYIRRGLPPSHAQYDFMERLDG |
| NOV7m | GTNFTLAELGICEPSPHRSGYCSDMGILHQGYSLSTGSDADSDTEGGMSPEHAIRLWGRG |
| NOV7n | GTNFTLAELGICEPSPHRSGYCSDMGILHQGYSLSTGSDADSDTEGGMSPEHAIRLWGRG |
| NOV7o | FSRKAFKLKKPSKYCSWKCAALSAIAAALLLAILLAYFIVPWSLKNSSIDSGEAEVGRRV |
| NOV7p | GTNFTLAELGICEPSPHRSGYCSDMGILHQGYSLSTGSDADSDTEGGMSPEHAIRLWGRG |
| NOV7q | DPSCGGHGSCIDGNCVCSAGYKGEHCEEVDCLDPTCSSHGVCVNGECLCSPGWGGLNCEL |
| NOV7r | RRVTQEVPPGVFWRSQIHISQPQFLKFNISLGKDALFGVYIRRGLPPSHAQYDFMERLDG |
| NOV7s | RRVTQEVPPGVFWRSQIHISQPQFLKFNISLGKDALFGVYIRRGLPPSHAQYDFMERLDG |
| NOV7t | GTNFTLAELGICEPSPHRSGYCSDMGILHQGYSLSTGSDADSDTEGGMSPEHAIRLWGRG |
| NOV7u | RRVTQEVPPGVFWRSQIHISQPQFLKFNISLGKDALFGVYIRRGLPPSHAQYDFMERLDG |
| NOV7v | RRVTQEVPPGVFWRSQIHISQPQFLKFNISLGKDALFGVYIRRGLPPSHAQYDFMERLDG |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | --------------------------------------------KNSSIDSGEAEVGRRV |
| NOV7ab | RRVTQEVPPGVFWRSQIHISQPQFLKFNISLGKDALFGVYIRRGLPPSHAQYDFMERLDG |
| NOV7ac | RRVTQEVPPGVFWRSQIHISQPQFLKFNISLGKDALFGVYIRRGLPPSHAQYDFMERLDG |
| NOV7a | TVYTPPPRLLPRNTFSRKAFKLKKPSKYCSWKCAALSAIAAALLLAILLAYFIVPWSLKN |
| NOV7b | KEKWSVVESPRERRSIQTLVQNEAVFVQYLDVGLWHLAFYNDGKDKEMVSFNTVVLDSVQ |
| NOV7c | ARVQCPDQCSGHGTYLPDTGLCSCDPNWMGPDCSVEVCSVDCGTHGVCIGGACRCEEGWT |
| NOV7d | ARVQCPDQCSGHGTYLPDTGLCSCDPNWMGPDCSVEVCSVDCGTHGVCIGGACRCEEGWT |
| NOV7e | KEKWSVVESPRERRSIQTLVQNEAVFVQYLDVGLWHLAFYNDGKDKEMVSFNTVVLDSVQ |
| NOV7f | TQEVPPGVFWRSQIHISQPQFLKFNISLGKDALFGVYIRRGLPPSHAQYDFMERLDGKEK |
| NOV7g | AECDVPMNQCIDPSCGGHGSCIDGNCVCSAGYKGEHCEEVDCLDPTCSSHGVCVNGECLC |
| NOV7h | -----------------MLHAANKGRKPSAEAGRPIPPTSSPSLLPSAQLPSSHNPPPVS |
| NOV7i | IKSRRSSGLSSRENSALTLTDSDNENKSDDENGRPIPPTSSPSLLPSAQLPSSHNPPPVS |
| NOV7j | IKSRRSSGLSSRENSALTLTDSDNENKSDDENGRPIPPTSSPSLLPSAQLPSSHNPPPVS |
| NOV7k | IKSRRSSGLSSRENSALTLTDSDNENKSDDENGRPIPPTSSPSLLPSAQLPSSHNPPPVS |
| NOV7l | KEKWSVVESPRERRSIQTLVQNEAVFVQYLDVGLWHLAFYNDGKDKEMVSFNTVVLDSVQ |
| NOV7m | IKSRRSSGLSSRENSALTLTDSDNENKSDDENGRPIPPTSSPSLLPSAQLPSSHNPPPVS |
| NOV7n | IKSRRSSGLSSRENSALTLTDSDNENKSDDENGRPIPPTSSPSLLPSAQLPSSHNPPPVS |
| NOV7o | TQEVPPGVFWRSQIHISQPQFLKFNISLGKDALFGVYIRRGLPPSHAQYDFMERLDGKEK |
| NOV7p | IKSRRSSGLSSRENSALTLTDSDNENKSDDENGRPIPPTSSPSLLPSAQLPSSHNPPPVS |
| NOV7q | ARVQCPDQCSGHGTYLPDTGLCSCDPNWMGPDCSVEVCSVDCGTHGVCIGGACRCEEGWT |
| NOV7r | KEKWSVVESPRERRSIQTLVQNEAVFVQYLDVGLWHLAFYNDGKDKEMVSFNTVVLDSVQ |
| NOV7s | KEKWSVVESPRERRSIQTLVQNEAVFVQYLDVGLWHLAFYNDGKDKEMVSFNTVVLDSVQ |
| NOV7t | IKSRRSSGLSSRENSALTLTDSDNENKSDDENGRPIPPTSSPSLLPSAQLPSSHNPPPVS |
| NOV7u | KEKWSVVESPRERRSIQTLVQNEAVFVQYLDVGLWHLAFYNDGKDKEMVSFNTVVLDSVQ |
| NOV7v | KEKWSVVESPRERRSIQTLVQNEAVFVQYLDVGLWHLAFYNDGKDKEMVSFNTVVLDSVQ |
| NOV7w | ------------------------------------------------------------ |
| NOV7x | ------------------------------------------------------------ |
| NOV7y | ------------------------------------------------------------ |
| NOV7z | -----------------------------------------------------------N |
| NOV7aa | TQEVPPGVFWRSQIHISQPQFLKFNISLGKDALFGVYIRRGLPPSHAQYDFMERLDGKEK |
| NOV7ab | KEKWSVVESPRERRSIQTLVQNEAVFVQYLDVGLWHLAFYNDGKDKEMVSFNTVVLDSVQ |
| NOV7ac | KEKWSVVESPRERRSIQTLVQNEAVFVQYLDVGLWHLAFYNDGKDKEMVSFNTVVLDSVQ |
| NOV7a | SSIDSGEAEVGRRVTQEVPPGVFWRSQIHISQPQFLKFNISLGKDALFGVYIRRGLPPSH |
| NOV7b | DCPRNCHGNGECVSGVCHCFPGFLGADCAKAACPVLCSGNGQYSKGTCQCYSGWKGAECD |
| NOV7c | GAACDQRVCHPRCIEHGTCKDGKCECREGWNGEHCTIGRQTAGTETDGCPDLCNGNGRCT |
| NOV7d | GAACDQRVCHPRCIEHGTCKDGKCECREGWNGEHCTIGRQTAGTETDGCPDLCNGNGRCT |
| NOV7e | DCPRNCHGNGECVSGVCHCFPGFLGADCAKAACPVLCSGNGQYSKGTCQCYSGWKGAECD |
| NOV7f | WSVVESPRERRSIQTLVQNEAVFVQYLDVGLWHLAFYNDGKDKEMVSFNTVVLDSVQDCP |
| NOV7g | SPGWGGLNCELARVQCPDQCSGHGTYLPDTGLCSCDPNWMGPDCSVDGCPDLCNGNGRCT |
| NOV7h | CQMPLLDSNTSHQIMDTNPDEEFSPNSYLLRACSGPQQASSSGPPNHHSQSTLRPPLPPP |
| NOV7i | CQMPLLDSNTSHQIMDTNPDEEFSPNSYLLRACSGPQQASSSGPPNHHSQSTLRPPLPPP |
| NOV7j | CQMPLLDSNTSHQIMDTNPDEEFSPNSYLLRACSGPQQASSSGPPNHHSQSTLRPPLPPP |
| NOV7k | CQMPLLDSNTSHQIMDTNPDEEFSPNSYLLRACSGPQQASSSGPPNHHSQSTLRPPLPPP |
| NOV7l | DCPRNCHGNGECVSGVCHCFPGFLGADCAKAACPVLCSGNGQYSKGTCQCYSGWKGAECD |
| NOV7m | CQMPLLDSNTSHQIMDTNPDEEFSPNSYLLRACSGPQQASSSGPPNHHSQSTLRPPLPPP |
| NOV7n | CQMPLLDSNTSHQIMDTNPDEEFSPNSYLLRACSGPQQASSSGPPNHHSQSTLRPPLPPP |
| NOV7o | WSVVESPRERRSIQTLVQNEAVFVQYLDVGLWHLAFYNDGKDKEMVSFNTVVLDSVQDCP |
| NOV7p | CQMPLLDSNTSHQIMDTNPDEEFSPNSYLLRACSGPQQASSSGPPNHHSQSTLRPPLPPP |
| NOV7q | GAACDQRVCHPRCIEHGTCKDGKCECREGWNGEHCTIGRQTAGTETDGCPDLCNGNGRCT |
| NOV7r | DCPRNCHGNGECVSGVCHCFPGFLGADCAKAACPVLCSGNGQYSKGTCQCYSGWKGAECD |
| NOV7s | DCPRNCHGNGECVSGVCHCFPGFLGADCAKAACPVLCSGNGQYSKGTCQCYSGWKGAECD |
| NOV7t | CQMPLLDSNTSHQIMDTNPDEEFSPNSYLLRACSGPQQASSSGPPNHHSQSTLRPPLPPP |
| NOV7u | DCPRNCHGNGECVSGVCHCFPGFLGADCAKAACPVLCSGNGQYSKGTCQCYSGWKGAECD |
| NOV7v | DCPRNCHGNGECVSGVCHCFPGFLGADCAKAACPVLCSGNGQYSKGTCQCYSGWKGAECD |
| NOV7w | ------------------------------------------------------------ |
| NOV7x | ------------------------------------------------------------ |
| NOV7y | ------------------------------------------------------------ |
| NOV7z | SSIDSGEAEVGRRVTQEVPPGVFWRSQIHISQPQFLKFNISLGKDALFGVYIRRGLPPSH |
| NOV7aa | WSVVESPRERRSIQTLVQNEAVFVQYLDVGLWHLAFYNDGKDKEMVSFNTVVLDSVQDCP |
| NOV7ab | DCPRNCHGNGECVSGVCHCFPGFLGADCAKAACPVLCSGNGQYSKGTCQCYSGWKGAECD |
| NOV7ac | DCPRNCHGNGECVSGVCHCFPGFLGADCAKAACPVLCSGNGQYSKGTCQCYSGWKGAECD |
| NOV7a | AQYDFMERLDGKEKWSVVESPRERRSIQTLVQNEAVFVQYLDVGLWHLAFYNDGKDKEMV |
| NOV7b | VPMNQCIDPSCGGHGSCIDGNCVCSAGYKGEHCEEVDCLDPTCSSHGVCVNGECLCSPGW |
| NOV7c | LGQNSWQCVCQTGWRGPGCNVAMETSCADNKDNEGDGLVDCLDPDCCLQSACQNSLLCRG |
| NOV7d | LGQNSWQCVCQTGWRGPGCNVAMETSCADNKDNEGDGLVDCLDPDCCLQSACQNSLLCRG |
| NOV7e | VPMNQCIDPSCGGHGSCIDGNCVCSAGYKGEHCEEVDCLDPTCSSHGVCVNGECLCSPGW |
| NOV7f | RNCHGNGECVSGVCHCFPGFLGADCAKAACPVLCSGNGQYSKGTCQCYSGWKGAECDVPM |
| NOV7g | LGQNSWQCVCQTGWRGPGCNVAMETSCADNKDNEGDGLVDCLDPDCCLQSACQNSLLCRG |
| NOV7h | HNHTLSHHHSSANSLNRNSLTNRRSQIHAPAPAPNDLATTPESVQLQDSWVLNSNVPLET |
| NOV7i | HNHTLSHHHSSANSLNRNSLTNRRSQIHAPAPAPNDLATTPESVQLQDSWVLNSNVPLET |
| NOV7j | HNHTLSHHHSSANSLNENSLTNRRSQIHAPAPAPNDLATTPESVQLQDSWVLNSNVPLET |
| NOV7k | HNHTLSHHHSSANSLNRNSLTNRRSQIHAPAPAPNDLATTPESVQLQDSWVLNSNVPLET |
| NOV7l | VPNNQCIDPSCGGHGSCIDGNCVCSAGYKGEHCEEVDCLDPTCSSHGVCVNGECLCSPGW |
| NOV7m | HNHTLSHHHSSANSLNRNSLTNRRSQIHAPAPAPNDLATTPESVQLQDSWVLNSNVPLET |
| NOV7n | HNHTLSHHHSSANSLNRNSLTNRRSQIHAPAPAPNDLATTPESVQLQDSWVLNSNVPLET |
| NOV7o | RNCHGNGECVSGVCHCFPGFLGADCAKAACPVLCSGNGQYSKGTCQCYSGWKGAECDVPM |
| NOV7p | HNHTLSHHHSSANSLNRNSLTNRRSQIHAPAPAPNDLATTPESVQLQDSWVLNSNVPLET |
| NOV7q | LGQNSWQCVCQTGWRGPGCNVAMETSCADNKDNEGDGLVDCLDPDCCLQSACQNSLLCRG |
| NOV7r | VPMNQCIDPSCGGHGSCIDGNCVCSAGYKGEHCEEVDCLDPTCSSHGVCVNGECLCSPGW |
| NOV7s | VPMNQCIDPSCGGHGSCIDGNCVCSAGYKGEHCEEVDCLDPTCSSHGVCVNGECLCSPGW |
| NOV7t | HNHTLSHHHSSANSLNRNSLTNRRSQIHAPAPAPNDLATTPESVQLQDSWVLNSNVPLET |
| NOV7u | VPMNQCIDPSCGGHGSCIDGNCVCSAGYKGEHCEEVDCLDPTCSSHGVCVNGECLCSPGW |
| NOV7v | VPMNQCIDPSCGGHGSCIDGNCVCSAGYKGEHCEEVDCLDPTCSSHGVCVNGECLCSPGW |
| NOV7w | ------------------------------------------------------------ |
| NOV7x | ------------------------------------------------------------ |
| NOV7y | ------------------------------------------------------------ |
| NOV7z | AQYDFMERLDGKEKWSVVESPRERRSIQTLVQNEAVFVQYLDVGLWHLAFYNDGKDKEMV |
| NOV7aa | RNCHGNGECVSGVCHCFPGFLGADCAKAACPVLCSGNGQYSKGTCQCYSGWKGAECDVPM |
| NOV7ab | VPMNQCIDPSCGGHGSCIDGNCVCSAGYKGEHCEEVDCLDPTCSSHGVCVNGECLCSPGW |
| NOV7ac | VPMNQCIDPSCGGHGSCIDGNCVCSAGYKGEHCEEVDCLDPTCSSHGVCVNGECLCSPGW |
| NOV7a | SFNTVVLDS-VQDCPRNCHGNGECVSGVCHCFPGFLGADCAKAACPVLCSGNGQYSKGTC |
| NOV7b | GGLNCELARVQCPDQCSGHGTYLPDTGLCSCDPNWMGPDCSVEVCSVDCGTHGVCIGGAC |
| NOV7c | SRDPLDIIQQGQTDWPAVKSFYDRIKLLAGKDSTHIIPGENPFNSSLVSLIRGQVVTTDG |
| NOV7d | SRDPLDIIQQGQTDWPAVKSFYDRIKLLAGKDSTHIIPGENPFNSSLVSLIRGQVVTTDG |
| NQV7e | GGLNCELARVQCPDQCSGHGTYLPDTGLCSCDPNWMGPDCSVEVCSVDCGTHGVCIGGAC |
| NOV7f | NQCIDPSCGGHGSCIDGNCVCSAGYKGEHCEEVDCLDPTCSSHGVCVNGECLCSPGWGGL |
| NOV7g | SRDPLDIIQQGQTDWPAVKSFYDRIKLLAGKDSTHIIPGENPFNSSLVSLIRGQVVTTDG |
| NOV7h | RHFLFKTSSGSTPLFSSSSPGYPLTSGTVYTPPPRLLPRNTFSRKAFKLKKPSKYCSWKC |
| NOV7i | RHFLFKTSSGSTPLFSSSSPGYPLTSGTVYTPPPRLLPRNTFSRKAFKLKKPSKYCSWKC |
| NOV7j | RHFLFKTSSGSTPLFSSSSPGYPLTSGTVYTPPPRLLPRNTFSRKAFKLKKPSKYCSWKC |
| NOV7k | RHFLFKTSSGSTPLFSSSSPGYPLTSGTVYTPPPRLLPRNTFSRKAFKLKKPSKYCSWKC |
| NOV7l | GGLNCELARVQCPDQCSGHGTYLPDTGLCSCDPNWMGPDCSVEVCSVDCGTHGVCIGGAC |
| NOV7m | RHFLFKTSSGSTPLFSSSSPGYPLTSGTVYTPPPRLLPRNTFSRKAFKLKKPSKYCSWKC |
| NOV7n | RHFLFKTSSGSTPLFSSSSPGYPLTSGTVYTPPPRLLPRNTFSRKAFKLKKPSKYCSWKC |
| NOV7o | NQCIDPSCGGHGSCIDGNCVCSAGYKGEHCEEVDCLDPTCSSHGVCVNGECLCSPGWGGL |
| NOV7p | RHFLFKTSSGSTPLFSSSSPGYPLTSGTVYTPPPRLLPRNTFSRKAFKLKKPSKYCSWKC |
| NOV7q | SRDPLDIIQQGQTDWPAVKSFYDRIKLLAGKDSTHIIPGENPFNSSLVSLIRGQVVTTDG |
| NOV7r | GGLNCELARVQCPDQCSGHGTYLPDTGLCSCDPNWMGPDCSVEVCSVDCGTHGVCIGGAC |
| NOV7s | GGLNCELARVQCPDQCSGHGTYLPDTGLCSCDPNWMGPDCSVEVCSVDCGTHGVCIGGAC |
| NOV7t | RHFLFKTSSGSTPLFSSSSPGYPLTSGTVYTPPPRLLPRNTFSRKAFKLKKPSKYCSWKC |
| NOV7u | GGLNCELARVQCPDQCSGHGTYLPDTGLCSCDPNWMGPDCSVEVCSVDCGTHGVCIGGAC |
| NOV7v | GGLNCELARVQCPDQCSGHGTYLPDTGLCSCDPNWMGPDCSVEVCSVDCGTHGVCIGGAC |
| NOV7w | ------------------------DTGLCSCDPNWMGPDCSVE----------------- |
| NOV7x | ----------------------DRIKLLAGKDSTHIIPGE-------------------- |
| NOV7y | ---------------------SAGYKGEHCEEVDCLDPTCS------------------- |
| NOV7z | SFNTVVLDDSVQDCPRNCHGNGECVSGVCHCFPGFLGADCAKDLPALTFCKTIINKAAL- |
| NOV7aa | NQCIDPSCGGHGSCIDGNCVCSAGYKGEHCEEVDCLDPTCSSHGVCVNGECLCSPGWGGL |
| NOV7ab | GGLNCELARVQCPDQCSGHGTYLPDTGLCSCDPNWMGPDCSVVCSVDCGTHGVCIGGACR |
| NOV7ac | GGLNCELARVQCPDQCSGHGTYLPDTGLCSCDPNWMGPDCSVEVCSVDCGTHGVCIGGAC |
| NOV7a | QCYSGWKGAECDVPMNQCIDPSCGGHGSCIDGNCVCSAGYKGEHCEEVDCLDPTCSSNGV |
| NOV7b | RCEEGWTGAACDQRVCHPRCIEHGTCKDGKCECREGWNGEHCTIDGCPDLCN-------- |
| NOV7c | TPLVGVNVSFVKYPKYGYTITRQDGTFDLIANGGASLTLHFERAPFMSQERTVWLPWNSF |
| NOV7d | TPLVGVNVSFVKYPKYGYTITRQDGTFDLIANGGASLTLHFERAPFMSQERTVWLPWNSF |
| NOV7e | RCEEGWTGAACDQRVCHPRCIEHGTCKDGKCECREGWNGEHCTIDGCPDLCNGNGRCTLG |
| NOV7f | NCELARVQCPDQCSGHGTYLPDTGLCSCDPNWMGPDCSVEVCSVDCGTHGVCIGGACRCE |
| NOV7g | TPLVGVNVSFVKYPKYGYTITRQDGTFDLIANGGASLTLHFERAPFMSQERTVWLPWNSF |
| NOV7h | AALSAIAAALLLAILLAYFIVPWSLKNSSIDSGEAEVGRRVTQEVPPGVFWRSQIHISQP |
| NOV7i | AALSAIAAALLLAIL--------------------------------------------- |
| NOV7j | AALSAIAAALLLAILLAYFIVPWSLKNSSIDSGEAEVGRRVTQEVPPGVFWRSQIHISQP |
| NOV7k | AALSAIAAALLLAIL--------------------------------------------- |
| NOV7l | RCEEGWTGAACDQRVCHPRCIEHGTCKDGKCECREGWNGEHCTIGRQTAGTETDGCPDLC |
| NOV7m | AALSAIAAALLLAILLAYFIVPWSLKNSSIDSGEAEVGRRVTQEVPPGVFWRSQIHISQP |
| NOV7n | AALSAIAAALLLAIL--------------------------------------------- |
| NOV7o | NCELARVQCPDQCSGHGTYLPDTGLCSCDPNWMGPDCSVEVCSVDCGTHGVCIGGACRCE |
| NOV7p | AALSAIAAALLLAILLAYFIVPWSLKNSSIDSGEAEVGRRVTQEVPPGVFWRSQIHISQP |
| NOV7q | TPLVGVNVSFVKYPKYGYTITRQDGTFDLIANGGASLTLHFERAPFMSQERTVWLPWNSF |
| NOV7r | RCEEGWTGAACDQRVCHPRCIEHGTCKDGKCECREGWNGEHCTIDGCPDLCN-------- |
| NOV7s | RCEEGWTGAACDQRVCHPRCIEHGTCKDGKCECREGWNGEHCTIGRQTAGTETDGCPDLC |
| NOV7t | AALSAIAAALLLAIL--------------------------------------------- |
| NOV7u | RCEEGWTGAACDQRVCHPRCIEHGTCKDGKCECREGWNGEHCTIGRQTAGTETDGCPDLC |
| NOV7v | RCEEGWTGAACDQRVCHPRCIEHGTCKDGKCECREGWNGEHCTIGRQTAGTETDGCPDLC |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | NCELARVQCPDQCSGHGTYLPDTGLCSCDPNWMGPDCSVEVCSVDCGTHGVCIGGACRCE |
| NOV7ab | CEEGWTGAACDQRVCHPRCIEHGTCKDGKCECREGWNGEHCTIDGCPDLCNG-------- |
| NOV7ac | RCEEGWTGAACDQRVCHPRCIEHGTCKDGKCECREGWNGEHCTIGRQTAGTETDGCPDLC |
| NOV7a | CVNGECLCSPGWGGLNCELARVQCPDQCSGHGTYLPDTGLCSCDPNWMGPDCSVDGCPDL |
| NOV7b | -GNGRCTLGQNSWQCVCQTGWRGPGCNVAMETSCADNKDNEGDGLVDCLDPDCCLQSACQ |
| NOV7c | YAMDTLVMKTEENSIPSCDLSGFVRPDPIIISSPLSTFFSAAPGQNPIVPETQVLHEEIE |
| NOV7d | YAMDTLVMKTEENSIPSCDLSGFVRPDPIIISSPLSTFFSAAPGQNPIVPETQVLHEEIE |
| NOV7e | QNSWQCVCQTGWRGPGCNVAMETSCADNKDNEGDGLVDCLDPDCCLQSACQNSLLCRGSR |
| NOV7f | EGWTGAACDQRVCHPRCIEHGTCKDGKCECREGWNGEHCTIDGCPDLCNGNGRCTLGQNS |
| NOV7g | YAMDTLVMKTEENSIPSCDLSGFVRPDPIIISSPLSTFFSAAPGQNPIVPETQVLHEEIE |
| NOV7h | QFLKFNISLGKDALFGVYIRRGLPPSHAQYDFMERLDGKEKWSVVESPRERRSIQTLVQN |
| NOV7i | ------------------------------------------------------------ |
| NOV7j | QFLKFNTSLGKDALFGVYIRRGLPPSHAQYDFMERLDGKEKWSVVESPRERRSIQTLVQN |
| NOV7k | ------------------------------------------------------------ |
| NOV7l | NGNGRCTLGQNSWQCVCQTGWRGPGCNVAMETSCADNKDNEGDGLVDCLDPDCCLQSACQ |
| NOV7m | QFLKFNISLGKDALFGVYIRRGLPPSHAQYDFMERLDGKEKWSVVESPRERRSIQTLVQN |
| NOV7n | ------------------------------------------------------------ |
| NOV7o | EGWTGAACDQRVCHPRCIEHGTCKDGKCECREGWNGEHCTIGRQTAGTETDGCPDLCNGN |
| NOV7p | QFLKFNISLGKDALFGVYIRRGLPPSHAQYDFMERLDGKEKWSVVESPRERRSIQTLVQN |
| NOV7q | YAMDTLVMKTEENSIPSCDLSGFVRPDPIIISSPLSTFFSAAPGQNPIVPETQVLHEEIE |
| NOV7r | -GNGRCTLGQNSWQCVCQTGWRGPGCNVAMETSCADNKDNEGDGLVDCLDPDCCLQSACQ |
| NOV7s | NGNGRCTLGQNSWQCVCQTGWRGPGCNVAMETSCADNKDNEGDGLVDCLDPDCCLQSACQ |
| NOV7t | ------------------------------------------------------------ |
| NOV7u | NGNGRCTLGQNSWQCVCQTGWRGPGCNVAMETSCADNKDNEGDGLVDCLDPDCCLQSACQ |
| NOV7v | NGNGRCTLGQNSWQCVCQTGWRGPGCNVAMETSCADNKDNEGDGLVDCLDPDCCLQSACQ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | EGWTGAACDQRVCHPRCIEHGTCKDGKCECREGWNGEHCTIGRQTAGTETDGCPDLCNGN |
| NOV7ab | ------NGR-CTLGQNSWQCVCQTGWRGPGCNVAMETSCADNKDNEGDGLIDCMDPDCCL |
| NOV7ac | NGNGRCTLGQNSWQCVCQTGWRGPGCNVAMETSCADNKDNEGDGLVDCLDPDCCLQSACQ |
| NOV7a | CNGNGRCTLGQNSWQCVCQTGWRGPGCNVAMETSCADNKDNEGDGLVDCLDPDCCLQSAC |
| NOV7b | N----------------------------------------------------------- |
| NOV7c | L----------------------------------------------------------- |
| NOV7d | L----------------------------------------------------------- |
| NOV7e | D----------------------------------------------------------- |
| NOV7f | W----------------------------------------------------------- |
| NOV7g | L----------------------------------------------------------- |
| NOV7h | EAVFVQYLDVGLWHLAFYNDGKDKEMVSFNTVVLDGTI---------------------- |
| NOV7i | ------------------------------------------------------------ |
| NOV7j | EAVFVQYLDVGLWHLAFYNDGKDKEMVSFNTVVLDS------------------------ |
| NOV7k | ------------------------------------------------------------ |
| NOV7l | N----------------------------------------------------------- |
| NOV7m | EAVFVQYLDVGLWHLAFYNDGKDKEMVSFNTVVLDSVQDCPRNCHGNGECVSGVCHCFPG |
| NOV7n | ------------------------------------------------------------ |
| NOV7o | G----------------------------------------------------------- |
| NOV7p | EAVFVQYLDVGLWHLAFYNDGKDKEMVSFNTVVLDSVQDCPRNCHGNGECVSGVCHCFPG |
| NOV7q | L----------------------------------------------------------- |
| NOV7r | N----------------------------------------------------------- |
| NOV7s | N----------------------------------------------------------- |
| NOV7t | ------------------------------------------------------------ |
| NOV7u | N----------------------------------------------------------- |
| NOV7v | N----------------------------------------------------------- |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | G----------------------------------------------------------- |
| NOV7ab | Q----------------------------------------------------------- |
| NOV7ac | N----------------------------------------------------------- |
| NOV7a | QNSLLCRGSRDPLDIIQQGQTDWPAVKSFYDRIKLLAGKDSTHIIPGENPFNSSLVSLIR |
| NOV7b | --SLLCRGSRDPLDIIQQGQTDWPAVKSFYDRIKLLAGKDSTHIIPGENPFNSSLVSLIR |
| NOV7c | --PGSNVKLRYLSSRTAGYKSLLKITMTQSTVPLNLIRVHLMVAVEGHLFQKSFQASPNL |
| NOV7d | --PGSNVKLRYLSSRTAGYKSLLKITMTQSTVPLNLIRVHLMVAVEGHLFQKSFQASPNL |
| NOV7e | --PLDIIQQGQTDWPAVKSFYDRIKLLAGKDSTHIIPGENPFNSSLVSLIRGQVVTTDGT |
| NOV7f | --QCVCQTGWRGPGCNVAMETSCADNKDNEGDGLVDCLDPDCCLQSACQNSLLCRGSRDP |
| NOV7g | --PGSNVKLRYLSSRTAGYKSLLKITMTQSTVPLNLIRVHLMVAVEGHLFQKSFQASPNL |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | --SLLCRGSRDPLDIIQQGQTDWPAVKSFYDRIKLLAGKDSTHIIPGENPFNSSLVSLIR |
| NOV7m | FLGADCAKGMCRHFP--------------------------------------------- |
| NOV7n | ------------------------------------------------------------ |
| NOV7o | --RCTLGQNSWQCVCQTGWRGPGCNVAMETSCADNKDNEGDGLVDCLDPDCCLQSACQNS |
| NOV7p | FLGADCAKAACPVLC--------------------------------------------- |
| NOV7q | --PGSNVKLRYLSSRTAGYKSLLKITMTQSTVPLNLIRVHLMVAVEGHLFQKSFQASPNL |
| NOV7r | --SLLCRGSRDPLDIIQQGQTDWPAVKSFYDRIKLLAGKDSTHIIPGENPFNSSLVSLIR |
| NOV7s | --SLLCRGSRDPLDIIQQGQTDWPAVKSFYDRIKLLAGKDSTHIIPGENPFNSSLVSLIR |
| NOV7t | ------------------------------------------------------------ |
| NOV7u | --SLLCRGSRDPLDIIQQGQTDWPAVKSFYDRIKLLAGKDSTHIIPGENPFNSSLVSLIR |
| NOV7v | --SLLCRGSRDPLDIIQQGQTDWPAVKSFYDRIKLLAGKDSTHIIPGENPFNSSLVSLIR |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | --RCTLGQNSWQCVCQTGWRGPGCNVAMETSCADNKDNEGDGLVDCLDPDCCLQSACQNS |
| NOV7ab | --SSCQNQPyCRGLPDPQDIISQSLQSPSQQAAKSFYDRISFLIGSDSTHVIPGESPFNS |
| NOV7ac | --SLLCRGSRDPLDIIQQGQTDWPAVKSFYDRIKLLAGKDSTHIIPGENPFNSSLVSLIR |
| NOV7a | GQVVTTDGTPLVGVNVSFVKYPKYGYTITRQDGTFDLIANGGASLTLHFERAPFMSQERT |
| NOV7b | GQVVTTDGTPLVGVNVSFVKYPKYGYTITRQDGTFDLIANGGASLTLHFERAPFMSQERT |
| NOV7c | ASTFIWDKTDAYGQRVYGLSDAVVSVGFEYETCPSLILWEKRTALLQGFELDPSNLGGWS |
| NOV7d | AYTFIWDKTDAYGQRVYGLSDAVVSVGFEYETCPSLILWEKRTALLQGFELDPSNLGGWS |
| NOV7e | PLVGVNVSFVKYPKYGYTITRQDGTFDLIANGGASLTLHFERAPFMSQERTVWLPWNSFY |
| NOV7f | LDIIQQGQTDWPAVKSFYDRIKLLAGKDSTHIIPGENPFNSSLVSLIRGQVVTTDGTPLV |
| NOV7g | AYTFIWDKTDAYGQRVYGLSDAVVSVGFEYETCPSLILWEKRTALLQGFELDPSNLGGWS |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | GQVVTTDGTPLVGVNVSFVKYPKYGYTITRQDGTFDLIANGGASLTLHFERAPFMSQERT |
| NOV7m | ------------------------------------------------------------ |
| NOV7n | ------------------------------------------------------------ |
| NOV7o | LLCRGSRDPLDIIQQGQTDWPAVKSFYDRIKLLAGKDSTHIIPGENPFNSSLVSLIRGQV |
| NOV7p | ------------------------------------------------------------ |
| NOV7q | ASTFIWDKTDAYGQRVYGLSDAVVSVGFEYETCPSLILWEKRTALLQGFELDPSNLGGWS |
| NOV7r | GQVVTTDGTPLVGVNVSFVKYPKYGYTITRQDGTFDLIANGGASLTLHFERAPFMSQERT |
| NOV7s | GQVVTTDGTPLVGVNVSFVKYPKYGYTITRQDGTFDLIANGGASLTLHFERAPFMSQERT |
| NOV7t | ------------------------------------------------------------ |
| NOV7u | GQVVTTDGTPLVGVNVSFVKYPKYGYTITRQDGTFDLIANGGASLTLHFERAPFMSQERT |
| NOV7v | GQVVTTDGTPLVGVNVSFVKYPKYGYTITRQDGTFDLIANGGASLTLHFERAPFMSQERT |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | LLCRGSRDPLDIIQQGQTDWPAVKSFYDRIKLLAGKDSTHIIPGENPFNSSLVSLIRGQV |
| NOV7ab | LVSLIRGQVVTTDGTPLVGVNVSFVKYPKYGYTITRQDGTYSLSRFDLIANGGASLTLHF |
| NOV7ac | GQVVTTDGTPLVGVNVSFVKYPKYGYTITRQDGTFDLIANGGASLTLHFERAPFMSQERT |
| NOV7a | VWLPWNSFYAMDTLVMKTEENSIPSCDLSGFVRPDPIIISSPLSTFFSAAPGQNPIVPET |
| NOV7b | VWLPWNSFYAMDTLVMKTEENSIPSCDLSGFVRPDPIIISSPLSTFFSAAPGQNPIVPET |
| NOV7c | LDKHHILNVKSGILHKGTGENQFLTQQPAIITSIMGNGRRRSISCPSCNGLAEGNKLLAP |
| NOV7d | LDKHHILNVKSGILHKGTGENQFLTQQPAIITSIMGNGRPRSISCPSCNGLAEGNKLLAP |
| NOV7e | AMDTLVMXTEENSIPSCDLSGFVRPDPIIISSPLSTFFSAAPGQNPIVPETQVLHEEIEL |
| NOV7f | GVNVSFVKYPKYGYTITRQDGTFDLIANGGASLTLHFEPAPFMSQERTVWLPWNSFYAMD |
| NOV7g | LDKHHILNVKSGILHKGTGENQFLTQQPAIITSIMGNGRRRSISCPSCNGLAEGNKLLAP |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | VWLPWNSFYAMDTLVMKTEENSIPSCDLSGFVRPDPIIISSPLSTFFSAAPGQNPIVPET |
| NOV7m | ------------------------------------------------------------ |
| NOV7n | ------------------------------------------------------------ |
| NOV7o | VTTDGTPLVGVNVSFVKYPKYGYTITRQDGTFDLIANGGASLTLHFERAPFMSQERTVWL |
| NOV7p | ------------------------------------------------------------ |
| NOV7q | LDKHHILNVKSGILHKGTGENQFLTQQPAIITSIMGNGRRRSISCPSCNGLAEGNKLLAP |
| NOV7r | VWLPWNSFYAMDTLVMKTEENSIPSCDLSGFVRPDPIIISSPLSTFFSAAPGQNPIVPET |
| NOV7s | VWLPWNSFYAMDTLVMKTEENSIPSCDLSGFVRPDPIIISSPLSTFFSAAPGQNPIVPET |
| NOV7t | ------------------------------------------------------------ |
| NOV7u | VWLPWNSFYAMDTLVMKTEENSIPSCDLSGFVRPDPIIISSPLSTFFSAAPGQNPIVPET |
| NOV7v | VWLPWNSFYAMDTLVMKTEENSIPSCDLSGFVRPDPIIISSPLSTFFSAAPGQNPIVPET |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | VTTDGTPLVGVNVSFVKYPKYGYTITRQDGTFDLIANGGASLTLHFEPAPFMSQERTVWL |
| NOV7ab | EPAPFMSQERTVWLPWNSFYANDTLVMKTEENSIPSCDLSGFCRLDPIIISSPLSTFFSA |
| NOV7ac | VWLPWNSFYAMDTLVMKTEENSIPSCDLSGFVRPDPIIISSPLSTFFSAAPGQNPIVPET |
| NOV7a | QVLHEEIELPGSNVKLRYLSSRTAGYKSLLKITMTQSTVPLNLIRVHLMVAVEGHLFQKS |
| NOV7b | QVLHEEIELPGSNVKLRYLSSRTAGYKSLLKITMTQSTVPLNLIRVHLMVAVEGHLFQKS |
| NOV7c | VALAVGIDGSLYVGDFNYIRRIFPSRN--------------------------------- |
| NOV7d | VALAVGIDGSLYVGDFNYIRRIFPSRNLEG------------------------------ |
| NOV7e | PGSNVKLRYLSSRTAGYKSLLKITMTQSTVPLNLIRVHLMVAVEGHLFQKSFQASPNLAY |
| NOV7f | TLVMKTEENSIPSCDLSGFVRPDPIIISSPLSTFFSAAPGQNPIVFETQVLHEEIELPGS |
| NOV7g | VALAVGIDGSLYVGDFNYIRRIFPSRNLEG------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | QVLHEEIELPGSNVKLRYLSSRTAGYKSLLKITMTQSTVPLNLIRVHLMVAVEGHLFQKS |
| NOV7m | ------------------------------------------------------------ |
| NOV7n | ------------------------------------------------------------ |
| NOV7o | PWNSFYAMDTLVMKTEENSIPSCDLSGFVRPDPIIISSFLSTFFSAAPGQNPIVPETQVL |
| NOV7p | ------------------------------------------------------------ |
| NOV7q | VALAVGIDGSLYVGDFNYIRRIFPSRNVTSILELRNKEFKHSNNPAHKYYLAVDPVSGSL |
| NOV7r | QVLHEEIELPGSNVKLRYLSSRTAGYKSLLKITMTQSTVPLNLIRVHLMVAVEGHLFQKS |
| NOV7s | QVLHEEIELPGSNVKLRYLSSRTAGYKSLLKITMTQSTVPLNLIRVHLMVAVEGHLFQKS |
| NOV7t | ------------------------------------------------------------ |
| NOV7u | QVLHEEIELPGSNVKLRYLSSRTAGYKSLLKITMTQSTVPLNLIRVHLMVAVEGHLFQKS |
| NOV7v | QVLHEEIELPGSNVKLRYLSSRTAGYKSLLKITMTQSTVPLNLIRVHLMVAVEGHLFQKS |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | PWNSFYAMDTLVMKTEENSIPSCDLSGFVRPDPIIISSPLSTFFSAAPGQNPIVPETQVL |
| NOV7ab | APGQNPIVPETQVLHEEIELPGSNVKLRYLSSRTAGYKSLLKITMTQSTVPLNLIRVHLM |
| NOV7ac | QVLHEEIELPGSNVKLRYLSSRTAGYKSLLKITMTQSTVPLNLIRVHLMVAVEGHLFQKS |
| NOV7a | FQASPNLASTFIWDKTDAYGQRVYGLSDAVVSVGFEYETCPSLILWEKRTALLQGFELDP |
| NOV7b | FQASPNLASTFIWDKTDAYGQRVYGLSDAVVSVGFEYETCPSLILWEKRTALLQGFELDP |
| NOV7c | ------------------------------------------------------------ |
| NOV7d | ------------------------------------------------------------ |
| NOV7e | TFIWDKTDAYGQRVYGLSDAVVSVGFEYETCPSLILWEKRTALLQGFELDPSNLGGWSLD |
| NOV7f | NVKLRYLSSRTAGYKSLLKITMTQSTVPLNLIRVHLMVAVEGHLFQKSFQASPNLAYTFI |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | FQASPNLASTFIWDKTDAYGQRVYGLSDAVVSVGFEYETCPSLILWEKRTALLQGFELDP |
| NOV7m | ------------------------------------------------------------ |
| NOV7n | ------------------------------------------------------------ |
| NOV7o | HEEIELPGSNVKLRYLSSRTAGYKSLLKITMTQSTVPLNLIRVHLMVAVEGHLFQKSFQA |
| NOV7p | ------------------------------------------------------------ |
| NOV7q | YVSDTNSRRIYRVKSLSGTKDLAGNSEVVAGTGEQCLPFDEARCGDGGKAIDATLMSPRG |
| NOV7r | FQASPNLASTFIWDKTDAYGQRVYGLSDAVVSVGFEYETCPSLILWEKRTALLQGFELDP |
| NOV7s | FQASPNLASTFIWDKTDAYGQRVYGLSDAVVSVGFEYETCPSLILWEKRTALLQGFELDP |
| NOV7t | ------------------------------------------------------------ |
| NOV7u | FQASPNLASTFIWDKTDAYGQRVYGLSDAVVSVGFEYETCPSLILWEKRTALLQGFELDP |
| NOV7v | FQASPNLAYTFIWDKTDAYGQRVYGLSDAVVSVGFEYETCPSLILWEKRTALLQGFELDP |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | HEEIELPGSNVKLRYLSSRTAGYKSLLKITMTQSTVPLNLTRVHLMVAVEGHLFQKSFQA |
| NOV7ab | VAVEGHLFQKSFQASPNLAYTFIWDKTDAYGQRVYGLSDAVGMFWFQRTALLQGFELDPS |
| NOV7ac | FQASPNASTFIWDKTDAYGQRVYGLSDAVVSVGFEYETCPSLILWEKRTALLQGFELDP |
| NOV7a | SNLGGWSLDKHHILNVKSGILHKGTGENQFLTQQPAITTSIMGNGRRRSISCPSCNGLAE |
| NOV7b | SNLGGWSLDKHHILNVKSGILHKGTGENQFLTQQPAIITSIMGNGRRRSISCPSCNGLAE |
| NOV7c | ------------------------------------------------------------ |
| NOV7d | ------------------------------------------------------------ |
| NOV7e | KHHILNVKSGILHKGTGENQFLTQQPAIITSIMGNGRRRSISCPSCNGLAEGNKLLAPVA |
| NOV7f | WDKTDAYGQRVYGLSDAVVSVGFEYETCPSLILWEKRTALLQGFELDPSNLGGWSLDKHH |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | SNLGGWSLDKHHILNVKSGILHKGTGENQFLTQQPAIITSIMGNGRRRSISCPSCNGLAE |
| NOV7m | ------------------------------------------------------------ |
| NOV7n | ------------------------------------------------------------ |
| NOV7o | SPNLASTFIWDKTDAYGQRVYGLSDAVVSVGFEYETCPSLILWEKRTALLQGFELDPSNL |
| NOV7p | ------------------------------------------------------------ |
| NOV7q | IAVDKNGLMYFVDATMIRKVDQNGIISTLLGSNDLTAVRPLSCDSSMDVAQVRLEWPTDL |
| NOV7r | SNLGGWSLDKHHILNVKSGILHKGTGENQFLTQQPAIITSIMGNGRRRSISCPSCNGLAE |
| NOV7s | SNLGGWSLDKHHILNVKSGTLHKGTGENQFLTQQPAIITSIMGNGRRRSISCPSCNGLAE |
| NOV7t | ------------------------------------------------------------ |
| NOV7u | SNLGGWSLDKHHILNVKSGILHKGTGENQFLTQQPAIITSIMGNGRRRSISCPSCNGLAE |
| NOV7v | SNLGGWSLDKHHILNVKSGILHKGTGENQFLTQQPAIITSIMGNGRPRSISCPSCNGLAE |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | SPNLAYTFIWDKTDAYGQRVYGLSDAVVSVGFEYETCPSLILWEKRTALLQGFELDPSNL |
| NOV7ab | NLGGWSLDKHHILNVKSGILHKGTGENQFLTQQPAIITSIMGNGRRRSISCPSCNGLAEG |
| NOV7ac | SNLGGWSLDKHHILNVKSGILHKGTGENQFLTQQPAIITSIMGNGRRRSISCPSCNGLAE |
| NOV7a | GNKLLAPVALAVGIDGSLYVGDFNYIRRIFPSRNVTSILELRNKEFKHSNNPAHKYYLAV |
| NOV7b | GNKLLAPVALAVGIDGSLYVGDFNYIRRIFPSRNVTSILELRNKEFKHSNNPAHKYYLAV |
| NOV7c | ------------------------------------------------------------ |
| NOV7d | ------------------------------------------------------------ |
| NOV7e | LAVGIDGSLYVGDFNYIRRIFPSRNLEG-------------------------------- |
| NOV7f | ILNVKSGILHKGTGENQFLTQQPAIITSIMGNGRRRSISCPSCNGLAEGNKLLAPVALAV |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | GNKLLAPVALAVGIDGSLYVGDFNYIRRIFPSRNVTSILELRNKEFKHSNNPAHKYYLAV |
| NOV7m | ------------------------------------------------------------ |
| NOV7n | ------------------------------------------------------------ |
| NOV7o | GGWSLDKHHILNVKSGILHKGTGENQFLTQQPAIITSIMGNGRRRSISCPSCNGLAEGNK |
| NOV7p | ------------------------------------------------------------ |
| NOV7q | AVNPMDNSLYVLENNVILRITENHQVSIIAGRPMHCQVPGIDYSLSKLAIHSALESASAI |
| NOV7r | GNKLLAPVALAVGIDGSLYVGDFNYIRRIFPSR1VTSILELRNKEFKHSNNPAHKYYLAV |
| NOV7s | GNKLLAPVALAVGIDGSLYVGDFNYIRRIFPSRNVTSILELRNKEFKHSNNPAHKYYLAV |
| NOV7t | ------------------------------------------------------------ |
| NOV7u | GNKLLAPVALAVGIDGSLYVGDFNYIRRIFPSRNVTSILELRNKEFKHSNNPAHKYYLAV |
| NOV7v | GNKLLAPVALAVGIDGSLYVGDFNYIRRIFPSRN-------------------------- |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | GGWSLDKHHILNVKSGILHKGTGENQFLTQQPAIITSIMGNGRRRSISCPSCNGLAEGNK |
| NOV7ab | NKLLAPVALAVGIDGSLYVGDFNYIRRIFPSRNVTSILELRRNKEFKHSNNPAHKYYLAV |
| NOV7ac | GNKLLAPVALAVGIDGSLYVGDFNYIRRIFPSRNVTSILELRNKEFKHSNNPAHKYYLAV |
| NOV7a | DPVSGSLYVSDTNSRRIYRVKSLSGTKDLAGNSEVVAGTGEQCLPFDEARCGDGGKAIDA |
| NOV7b | DPVSGSLYVSDTNSRRIYRVKSLSGTKDLAGNSEVVAGTGEQCLPFDEARCGDGGKAIDA |
| NOV7c | ------------------------------------------------------------ |
| NOV7d | ------------------------------------------------------------ |
| NOV7e | ------------------------------------------------------------ |
| NOV7f | GIDGSLYVGDFNYIRRIFPSRNLEG----------------------------------- |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | DPVSGSLYVSDTNSRRIYRVKSLSGTKDLAGNSEVVAGTGEQCLPFDEARCGDGGKAIDA |
| NOV7m | ------------------------------------------------------------ |
| NOV7n | ------------------------------------------------------------ |
| NOV7o | LLAPVALAVGIDGSLYVGDFNYIRRIFPSRNVTSILELRNKEFKHSNNPAHKYYLAVDPV |
| NOV7p | ------------------------------------------------------------ |
| NOV7q | AISHTGVLYITETDEKKINRLRQVTTNGEICLLAGAASDCDCKNDVNCNCYSGDDAYATD |
| NOV7r | DPVSGSLYVSDTNSRRIYRVKSLSGTKDLAGNSEVVAGTGEQCLPFDEARCGDGGKAIDA |
| NOV7s | DPVSGSLYVSDTNSRRIYRVKSLSGTKDLAGNSEVVAGTGEQCLPFDEARCGDGGKAIDA |
| NOV7t | ------------------------------------------------------------ |
| NOV7u | DPVSGSLYVSDTNSRRIYRVKSLSGTKDLAGNSEVVAGTGEQCLPFDEARCGDGGKAIDA |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | LLAPVALAVGIDGSLYVGDFNYIRRIFPSRN----------------------------- |
| NOV7ab | DPVSGSLYVSDTNSRRIYRVKSLSGTKDLAGNSEVVAGTGEQCLPFDEARCGDGGKAIDA |
| NOV7ac | DPVSGSLYVSDTNSRRIYRVKSLSGTKDLAGNSEVVAGTGEQCLPFDEARCGDGGKAIDA |
| NOV7a | TLMSPRGIAVDKNGLMYFVDATMIRXVDQNGIISTLLGSNDLTAVRPLSCDSSMDVAQVR |
| NOV7b | TLMSPRGIAVDKNGLMYFVDATMIRKVDQNGIISTLLGSNDLTAVRPLSCDSSMDVAQVR |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | TLMSPRGIAVDKNGLMYFVDATMIRKVDQNGIISTLLGSNDLTAVRPLSCDSSMDVAQVR |
| NOV7m | ------------------------------------------------------------ |
| NOV7n | ------------------------------------------------------------ |
| NOV7o | SGSLYVSDTNSRRIYRVKSLSGTKDLAGNSEVVAGTGEQCLPFDEARCGDGGKAIDATLM |
| NOV7p | ------------------------------------------------------------ |
| NOV7q | AILNSPSSLAVAPDGTIYIADLGNIRIRAVSKNKPVLNAFNQYEAASPGEQELYVFNADG |
| NOV7r | TLMSPRGIAVDKNGLMYFVDATMIRKVDQNGIISTLLGSNDLTAVRPLSCDSSMDVAQVR |
| NOV7s | TLMSPRGIAVDKNGLMYFVDATMIRKVDQNGIISTLLGSNDLTAVRPLSCDSSMDVAQVR |
| NOV7t | ------------------------------------------------------------ |
| NOV7u | TLMSPRGIAVDKNGLMYFVDATMIRKVDQNGIISTLLGSNDLTAVRPLSCDSSMDVAQVR |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | TLMSPRGIAVDKNGLMYFVDATMIRKVDQNGIISTLLGSNDLTAVRPLSCDSSMDVAQVR |
| NOV7ac | TLMSPRGIAVDKNGLMYFVDATMIRKVDQNGIISTLLGSNDLTAVRPLSCDSSMDVAQVR |
| NOV7a | LEWPTDLAVNPMDNSLYVLENNVILRITENHQVSIIAGRPMHCQVPGIDYSLSKIAIHSA |
| NOV7b | LEWPTDLAVNPMDNSLYVLENNVILRITENHQVSIIAGRPMHCQVPGIDYSLSKIAIHSA |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | LEWPTDIAVNPMDNSLYVLENNVILRITENHQVSIIAGRPMHCQVPGIDYSLSKLAIHSA |
| NOV7m | ------------------------------------------------------------ |
| NOV7n | ------------------------------------------------------------ |
| NOV7o | SPRGIAVDKNGLMYFVDATMIRKVDQNGIISTLLGSNDLTAVRPLSCDSSMDVAQVRLEW |
| NOV7p | ------------------------------------------------------------ |
| NOV7q | IHQYTVSLVTGEYLYNFTYSTDNDVTELIDNNGNSLKIRRDSSGMPRHLLMPDNQIITLT |
| NOV7r | LEWPTDLAVNPMDNSLYVLENNVILRITENHQVSIIAGRPMHCQVPGIDYSLSKLAIHSA |
| NOV7s | LEWPTDLAVNPMDNSLYVLENNVILRITENHQVSIIAGRPMHCQVPGIDYSLSKTAIHSA |
| NOV7t | ------------------------------------------------------------ |
| NOV7u | LEWPTDLAVNPMDNSLYVLENNVILRITENHQVSIIAGRFMHCQVPGIDYSLSKLAIHSA |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | LEWPTDLAVNPMDNSLYVLENNVILRITENHQVSIIAGRPMHCQVPGIDYSLSKLAIHSA |
| NOV7ac | LEWPTDLAVNPMDNSLYVLENNVILRITENHQVSIIAGRPMHCQVPGIDYSLSKLAIHSA |
| NOV7a | LESASAIAISHTGVLYITETDEKKINRLRQVTTNGEICLLAGAASDCDCKNDVNCNCYSG |
| NOV7b | LESASAIAISHTGVLYITETDEKKINRLRQVTTNGEICLLAGAASDCDCKNDVNCNCYSG |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | LESASAIAISHTGVLYITETDEKKINRLRQVTTNGEICLLAGAASDCDCKNDVNCNCYSG |
| NOV7m | ------------------------------------------------------------ |
| NOV7n | ------------------------------------------------------------ |
| NOV7o | PTDLAVNPMDNSLYVLENNVILRITENHQVSIIAGRPMHCQVPGIDYSLSKLAIHSALES |
| NOV7p | ------------------------------------------------------------ |
| NOV7q | VGTNGGLKVVSTQNLELGLMTYDGNTGLLATKSDETGWTTFYDYDHEGRLTNVTRPTGVV |
| NOV7r | LESASAIAISHTGVLYITETDEKKINRLRQVTTNGEICLLAGAASDCDCKNDVNCNCYSG |
| NOV7s | LESASAIAISHTGVLYITETDEKKINRLRQVTTNGEICLLAGAASDCDCKNDVNCNCYSG |
| NOV7t | ------------------------------------------------------------ |
| NOV7u | LESASAIAISHTGVLYITETDEKKINRLRQVTTNGEICLLAGAASDCDCKNDVNCNCYSG |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | LESASAIAISHTGVLYITETDEKKINRLRQVTTNGEICLLAGAASDCDCKNDVNCNCYSG |
| NOV7ac | LESASAIAISHTGVLYITETDEKKINRLRQVTTNGEICLLAGAASDCDCKNDVNCNCYSG |
| NOV7a | DDAYATDAILNSPSSLAVAPDGTIYIADLGNIRIRAVSKNKPVLNAFNQYEAASPGEQEL |
| NOV7b | DDAYATDAILNSPSSLAVAPDGTIYIADLGNIRIRAVSKNKPVLNAFNQYEAASPGEQEL |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | DDAYATDAILNSPSSLAVAPDGTIYIADLGNIRIRAVSKNKPVLNAFNQYEAASPGEQEL |
| NOv7m | ------------------------------------------------------------ |
| NOV7n | ------------------------------------------------------------ |
| NOV7o | ASAIAISHTGVLYITETDEKKINRLRQVTTNGEICLLAGAASDCDCKNDVNCNCYSGDDA |
| NOV7p | ------------------------------------------------------------ |
| NOV7q | TSLHREMEKSITIDIENSNRDDDVTVITNLSSVEASYTVVQDQVRNSYQLCNNGTLRVMY |
| NOV7r | DDAYATDAILNSPSSLAVAPDGTIYIADLGNIRIRAVSKNKPVLNAFNQYEAASPGEQEL |
| NOV7s | DDAYATDAILNSPSSLAVAPDGTIYIADLGNIRIPAVSKNKPVLNAFNQYEAASPGEQEL |
| NOV7t | ------------------------------------------------------------ |
| NOV7u | DDAYATDAILNSPSSLAVAPDGTIYIADLGNIRIRAVSKNKPVLNAFNQYEAASPGEQEL |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | DDAYATDAILNSPSSLAVAPDGTIYIADLGNIRIRAVSKNKPVLNAFNQYEAASPGEQEL |
| NOV7ac | DDAYATDAILNSPSSLAVAPDGTIYIADLGNIRIRAVSKNKPVLNAFNQYEAASPGEQEL |
| NOV7a | YVFNADGIHQYTVSLVTGEYLYNFTYSTDNDVTELIDNNGNSLKIRRDSSGMPRHLLMPD |
| NOV7b | YVFNADGIHQYTVSLVTGEYLYNFTYSTDNDVTELIDNNGNSLKIRRDSSGMPRHLLMPD |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | YVFNADGIHQYTVSLVTGEYLYNFTYSTDNDVTELIDNNGNSLKIRRDSSGMPRHLLMPD |
| NOV7m | ------------------------------------------------------------ |
| NOV7n | ------------------------------------------------------------ |
| NOV7o | YATDAILNSPSSLLAVAPDGTIYIADLGNIRIRAVSIKPVLNAFNQYEAASPGEQELYVF |
| NOV7p | ------------------------------------------------------------ |
| NOV7q | ANGMGISFHSEPHVLAGTITPTIGRCNISLPMENGLNSIEWRLRKEQIKGKVTIFGRKLR |
| NOV7r | YVFNADGIHQYTVSLVTGEYLYNFTYSTDNDVTELIDNNGNSLKIRRDSSGMPRHLLMPD |
| NOV7s | YVFNADGIHQYTVSLVTGEYLYNFTYSTDNDVTELIDNNGNSLKIRRDSSGMPRHLLMPD |
| NOV7t | ------------------------------------------------------------ |
| NOV7u | YVFNADGIHQYTVSLVTGEYLYNFTYSTDNDVTELIDNNGNSLKIPRDSSGMPRHLLMPD |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | YVFNADGIHQYTVSLVTGEYLYNFTYSTDNDVTELIDNNGNSLKIRRDSSGMPRHLLMPD |
| NOV7ac | YVFNADGIHQYTVSLVTGEYLYNFTYSTDNDVTELIDNNGNSLKIRRDSSGMPRHLLMPD |
| NOV7a | NQIITLTVGTNGGLKVVSTQNLELGLMTYDGNTGLLATKSDETGWTTFYDYDHEGRLTNV |
| NOV7b | NQIITLTVGTNGGLKVVSTQNLELGLMTYDGNTGLLATKSDETGWTTFYDYDHEGRLTNV |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | NQIITLTVGTNGGLKVVSTQNLELGLMTYDGNTGLLATKSDETGWTTFYDYDHEGRLTNV |
| NOV7m | ------------------------------------------------------------ |
| NOV7n | ------------------------------------------------------------ |
| NOV7o | NADGIHQYTVSLVTGEYLYNFTYSTDNDVTELIDNNGNSLKIRRDSSGMPRHLLMPDNQI |
| NOV7p | ------------------------------------------------------------ |
| NOV7q | VHGRNLLSIDYDRNIRTEKIYDDHRKFTLRIIYDQVGRPFLWLPSSGLAAVNVSYFFNGR |
| NOV7r | NQIITLTVGTNGGLKVVSTQNLELGLMTYDGNTGLLATKSDETGWTTFYDYDHEGRLTNV |
| NOV7s | NQIITLTVGTNGGLKVVSTQNLELGLMTYDGNTGLLATKSDETGWTTFYDYDHEGRLTNV |
| NOV7t | ------------------------------------------------------------ |
| NOV7u | NQIITLTVGTNGGLKVVSTQNLELGLMTYDGNTGLLATKSDETGWTTFYDYDHEGRLTNV |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | NQIITLTVGTNGGLKVVSTQNLELGLMTYDGNTGLLATKSDETGWTTFYSYDHEGRLTNV |
| NOV7ac | NQIITLTVGTNGGLKVVSTQNLELGLMTYDGNTGLLATKSDETGWTTFYDYDNEGRLTNV |
| NOV7a | TRPTGVVTSLHREMEKSITIDIENSNRDDDVTVITNLSSVEASYTVVQDQVRNSYQLCNN |
| NOV7b | TRPTGVVTSLHREMEKSITIDIENSNRDDDVTVITNLSSVEASYTVVQDQVPNSYQLCNN |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | TRPTGVVTSLHREMEKSITIDIENSNRDDDVTVITNLSSVEASYTVVQDQVRNSYQLCNN |
| NOV7m | ------------------------------------------------------------ |
| NOV7n | ------------------------------------------------------------ |
| NOV7o | ITLTVGTNGGLKVVSTQNLELGLMTYDGNTGLLATKSDETGWTTFYDYDHEGRLTNVTRP |
| NOV7p | ------------------------------------------------------------ |
| NOV7q | LAGLQRGANSERTDIDKQGRIVSRMFADGKVWSYSYLDKSMVLLLQSQRQYIFEYDSSDR |
| NOV7r | TRPTGVVTSLHREMEKSITIDIENSNRDDDVTVITNLSSVEASYTVVQDQVRNSYQLCNN |
| NOV7s | TRPTGVVTSLHREMEKSITIDIENSNRDDDVTVITNLSSVEASYTVVQDQVPNSYQLCNN |
| NOV7t | ------------------------------------------------------------ |
| NOV7u | TRPTGVVTSLHREMEKSITIDIENSNRDDDVTVITNLSSVEASYTWQDQVRNSYQLCNN |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | TRPTGVVTSLHREMEKSITIDIENSNRDDDVTVITNLSSVEASYTVVQDQVRNSYQLCNN |
| NOV7ac | TRPTGVVTSLHREMEKSITIDIENSNRDDDVTVITNLSSVEASYTVVQDQVRNSYQLCNN |
| NOV7a | GTLRVMYANGMGISFHSEPHVLAGTITPTIGRCNISLPMENGLNSIEWRLRKEQIKGKVT |
| NOV7b | GTLRVMYANGMGISFHSEPHVLAGTITPTIGRCNISLPMENGLNSIEWRLRKEQIKGKVT |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7l | ------------------------------------------------------------ |
| NOV7k | ------------------------------------------------------------ |
| NOV7l | GTLRVMYANGMGISFHSEPHVLAGTITPTIGRCNISLPMENGLNSIEWRLRKEQIKGKVT |
| NOV7m | ------------------------------------------------------------ |
| NOV7n | ------------------------------------------------------------ |
| NOV7o | TGVVTSLHREMEKSITIDIENSNRDDDVTVITNLSSVEASYTVVQDQVPNSYQLCNNGTL |
| NOV7p | ------------------------------------------------------------ |
| NOV7q | LLAVTMPSVAPHSMSTHTSIGYIRNIYNPPESNASVIFDYSDDGRILKTSFLGTGRQVFY |
| NOV7r | GTLRVMYANGMGISFHSEPHVLAGTITPTIGRCNISLPMENGLNSIEWRLRKEQIKGKVT |
| NOV7s | GTLRVMYANGMGISFHSEPHVLAGTITPTIGRCNISLPMENGLNSIEWRLRKEQIKGKVT |
| NOV7t | ------------------------------------------------------------ |
| NOV7u | GTLRVMYANGMGISFHSEPHVLAGTITPTIGRCNISLPMENGLNSIEWRLRKEQIKGKVT |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | GTLRVMYANGMGISFHSEPHVLAGTITPTIGRCNISLPMENGLNSIEWRLRKEQIKGKVT |
| NOV7ac | GTLRVMYANGMGISFHSEPNVLAGTITPTIGRCNISLPMENGLNSIEWRLRKEQIKGKVT |
| NOV7a | IFGRKLRVHGRNLLSIDYDRNIRTEKIYDDHRKFTLRIIYDQVGRPFLWLPSSGLAAVNV |
| NOV7b | IFGRKLRVHGRNLLSIDYDRNIRTEKIYDDHRKFTLRIIYDQVGRPFLWLPSSGLAAVNV |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7l | ------------------------------------------------------------ |
| NOV7k | ------------------------------------------------------------ |
| NOV7l | IFGRKLRVHGRNLLSIDYDRNIRTEKIYDDHRKFTLRIIYDQVGRPFLWLPSSGLAAVNV |
| NOV7m | ------------------------------------------------------------ |
| NOV7n | ------------------------------------------------------------ |
| NOV7o | RVMYANGMGISFHSEPHVIAGTITPTIGRCNISLPMENGLNSIEWRLRKEQIKGKVTIFG |
| NOV7p | ------------------------------------------------------------ |
| NOV7q | KYGKLSKLSEIVYDSTAVTFGYDETTGVLKMVNLQSGGFSCTIRYRKIGPLVDKQIYRFS |
| NOV7r | IFGRKLRVHGRNLLSIDYDRNIRTEKIYDDHRKFTLRIIYDQVGRPFLWLPSSGLAAVNV |
| NOV7s | IFGRKLRVHGRNLLSIDYDRNIRTEKIYDDHRKFTLRIIYDQVGRPFLWLPSSGLAAVNV |
| NOV7t | ------------------------------------------------------------ |
| NOV7u | IFGRKLRVHGRNLLSIDYDRNIRTEKIYDDHRKFTLRIIYDQVGRPFLWLPSSGLAAVNV |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | IFGRKLEVHGRNLLSIDYDRNIRTEKIYDDHRKFTLRIIYDQVGRPFLWLPSSGLAAVNV |
| NOV7ac | IFGRKLRVHGRNLLSIDYDRNIRTEKIYDDHRKFTLRIIYDQVGRPFLWLPSSGLAAVNV |
| NOV7a | SYFFNGRLAGLQRGAMSERTDIDKQGRIVSRMFADGKVWSYSYLDKSMVLLLQSQRQYIF |
| NOV7b | SYFFNGRLAGLQRGAMSERTDIDKQGRIVSRMFADGKVWSYSYLDKSMVLLLQSQRQYIF |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | SYFFNGRLAGLQRGAMSERTDIDKQGRIVSRMFADGKVWSYSYLDKSMVLLLQSQRQYIF |
| NOV7m | ------------------------------------------------------------ |
| NOV7n | ------------------------------------------------------------ |
| NOV7o | RKLRVHGRNLLSIDYDRNIRTEKIYDDHRKFTLRIIYDQVGRPFLWLPSSGLAAVNVSYF |
| NOV7p | ------------------------------------------------------------ |
| NOV7q | EEGMVNARFDYTYHDNSFRIASIKPVISETPLPVDLYRYDEISGKVEHFGKFGVIYYDIN |
| NOV7r | SYFFNGRLAGLQRGAMSERTDIDKQGRIVSRMFADGKVWSYSYLDKSMVLLLQSQRQYIF |
| NOV7s | SYFFNGRLAGLQRGAMSERTDIDKQGRIVSRMFADGKVWSYSYLDKSMVLLLQSQRQYIF |
| NOV7t | ------------------------------------------------------------ |
| NOV7u | SYFFNGRLAGLQRGANSERTDIDKQGRIVSRNFADGKVWSYSYLDKSMVLLLQSQRQYIF |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | SYFFNGRLAGLQRGAMSERTDIDKQGRIVSRNFADGKVWSYSYLDKMVLLLQSQRQYIFE |
| NOV7ac | SYFFNGRLAGLQRGAMSERTDIDKQGRIVSPMFADGKVWSYSYLDKSMVLLLQSQRQYIF |
| NOV7a | EYDSSDRLLAVTMPSVARHSMSTHTSIGYIRNIYNPPESNASVIFDYSDDGRILKTSFLG |
| NOV7b | EYDSSDRLLAVTMPSVARHSMSTHTSIGYIPNIYNPPESNASVIFDYSDDGRILKTSFLG |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | EYDSSDRLLAVTMPSVARHSMSTHTSIGYIRNIYNPPESNASVIFDYSDDGRILKTSFLG |
| NOV7m | ------------------------------------------------------------ |
| NOV7n | ------------------------------------------------------------ |
| NOV7o | FNGRLAGLQRGAMSERTDIDKQGRIVSRMFADGKVWSYSYLDKSMVLLLQSQRQYIFEYD |
| NOV7p | ------------------------------------------------------------ |
| NOV7q | QIITTAVMTLSKHFDTHGRIKEVQYEMFRSLMYWMTVQYDSMGRVIKRELKLGPYANTTK |
| NOV7r | EYDSSDRLLAVTMPSVARHSMSTHTSIGYIRMIYNPPESNASVIFDYSDDGRILKTSFLG |
| NOV7s | EYDSSDRLLAVTMPSVARHSMSTHTSIGYIRNIYNPPESNASVIFDYSDDGRILKTSFLG |
| NOV7t | ------------------------------------------------------------ |
| NOV7u | EYDSSDRLLAVTMPSVARHSMSTHTSIGYIRNIYNPPESNASVIFDYSDDGRILKTSFLG |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | YDSSDRLLAVTMPSVARHSMSTHTSIGYIRNIYNPPESNASVIFDYSDDGRILKTSFLGT |
| NOV7ac | EYDSSDRLLAVTMPSVARHSMSTHTSIGYIRNIYNPPESNASVIFDYSDDGRILKTSFLG |
| NOV7a | TGRQVFYKYGKLSKLSEIVYDSTAVTFGYDETTGVLKMVNLQSGGFSCTIRYRKIGPLVD |
| NOV7b | TGRQVFYKYGKLSKLSEIVYDSTAVTFGYDETTGVLKMVNLQSGGFSCTIRYRKIGPLVD |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | TGRQVFYKYGKLSKLSEIVYDSTAVTFGYDETTGVLKMVNLQSGGFSCTIRYRKIGPLVD |
| NOV7m | ------------------------------------------------------------ |
| NOV7n | ------------------------------------------------------------ |
| NOV7o | SSDRLLAVTMPSVARHSMSTHTSIGYIRNIYNPPESNASVIFDYSDDGRILKTSFLGTGR |
| NOV7p | ------------------------------------------------------------ |
| NOV7q | YTYDYDGDGQLQSVAVNDRPTWRYSYDLNGNLHLLNPGNSVRLMPLRYDLRDRITRLGDV |
| NOV7r | TGRQVFYKYGKLSKLSEIVYDSTAVTFGYDETTGVLKMVNLQSGGFScTIRYRKIGPLVD |
| NOV7s | TGRQVFYKYGKLSKLSEIVYDSTAVTFGYDETTGVLKMVNLQSGGFSCTIRYRKIGPLVD |
| NOV7t | ------------------------------------------------------------ |
| NOV7u | TGRQVFYKYGKLSKLSEIVYDSTAVTFGYDETTGVLKMVNLQSGGFSCTIRYRKIGPLVD |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | GRQVFYKYGKLSKLSEIVYDSTAVTFGYDETTGVLKMVNLQSGGFSCTIRYRKIGPLVDK |
| NOV7ac | TGRQVFYKYGKLSKLSEIVYDSTAVTFGYDETTGVLKMVNLQSGGFSCTIRYRKIGPLVD |
| NOV7a | KQIYRFSEEGMVNARFDYTYHDNSFRIASIKPVISETPLPVDLYRYDEISGKVEHFGKFG |
| NOV7b | KQIYRFSEEGMVNARFDYTYHDNSFRIASIKPVISETPLPVDLYRYDEISGKVEHFGKFG |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | KQIYRFSEEGMVNARFDYTYHDNSFRIASIKPVISETPLPVDLYRYDEISGKVEHFGKFG |
| NOV7m | ------------------------------------------------------------ |
| NOV7n | ------------------------------------------------------------ |
| NOV7o | QVFYKYGKLSKLSEIVYDSTAVTFGYDETTGVLKMVNLQSGGFSCTIRYRKIGPLVDKQI |
| NOV7p | ------------------------------------------------------------ |
| NOV7q | QYKIDDDGYLCQRGSDIFEYNSKGLLTRAYNKASGWSVQYRYDGVGRRASYKTNLGHHLQ |
| NOV7r | KQIYRFSEEGMVNARFDYTYHDNSFRIASIKPVISETPLPVDLYRYDEISGKVEHFGKFG |
| NOV7s | KQIYRFSEEGMVNARFDYTYHDNSFRIASIKPVISETPLPVDLYRYDEISGKVEHFGKFG |
| NOV7t | ------------------------------------------------------------ |
| NOV7u | KQIYRFSEEGMVNARFDYTYHDNSFRIASIKPVISETPLPVDLYRYDEISGKVEHFGKFG |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | QIYRFSEEGMVNARFDYTYHDNSFRIASIKPVISETPLPVDLYRYDEISGKVEHFGKFGV |
| NOV7ac | KQIYRFSEEGMVNARFDYTYHDNSFRIASIKPVISETPLPVDLYRYDETSGKVEHFGKFG |
| NOV7a | VIYYDINQIITTAVMTLSKHFDTHGRIKEVQYEMFRSLMYWMTVQYDSMGRVIKRELKLG |
| NOV7b | VIYYDINQIITTAVMTLSKHFDTHGRIKEVQYEMFRSLMYWMTVQYDSMGRVIKRELKLG |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | VIYYDINQIITTAVMTLSKHFDTHGRIKEVQYEMFRSLMYWMTVQYDSMGRVIKRELKLG |
| NOV7m | ------------------------------------------------------------ |
| NOV7n | ------------------------------------------------------------ |
| NOV7o | YRFSEEGMVNARFDYTYHDNSFRIASIKPVISETPLPVDLYRYDEISGKVEHFGKFGVIY |
| NOV7p | ------------------------------------------------------------ |
| NOV7q | YFYSDLHNPTRITHVYNHSNSEITSLYYDLQGHLFAMESSSGEEYYVASDNTGTPLAVFS |
| NOV7r | VIYYDINQIITTAVMTLSKHFDTHGRIKEVQYEMFRSLMYWMTVQYDSMGRVIKRELKLG |
| NOV7s | VIYYDINQIITTAVMTLSKHFDTHGRIKEVQYEMFRSLMYWMTVQYDSMGRVIKRELKLG |
| NOV7t | ------------------------------------------------------------ |
| NOV7u | VIYYDINQIITTAVMTLSKHFDTHGRIKEVQYEMFRSLMYWMTVQYDSMGRVIKRELKLG |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | IYYDINQIITTAVMTLSKHFDTHGRIKEVQYEMFRSLMYWMTVQYDSMGRVIKRELKLGP |
| NOV7ac | VIYYDINQIITTAVMTLSKHFDTHGRIKEVQYEMFRSLMYWMTVQYDSMGRVIKRELKLG |
| NOV7a | PYANTTKYTYDYDGDGQLQSVAVNDRPTWRYSYDLNGNLHLLNPGNSVRLMPLRYDLFLR |
| NOV7b | PYANTTKYTYDYDGDGQLQSVAVNDRPTWRYSYDLNGNLHLLNPGNSVRLMPLRYDLFLR |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | PYANTTKYTYDYDGDGQLQSVAVNDRPTWRYSYDLNGNLHLLNPGNSVRLMPLRYDLRDR |
| NOV7m | ------------------------------------------------------------ |
| NOV7n | ------------------------------------------------------------ |
| NOV7o | YDINQIITTAVMTLSKHFDTHGRIKEVQYEMFRSLMYWMTVQYDSMGRVIKRELKLGPYA |
| NOV7p | ------------------------------------------------------------ |
| NOV7q | INGLMIKQLQYTAYGEIYYDSNPDFQMVIGFHGGLYDPLTKLVHFTQRDYDVLAGRWTSP |
| NOV7r | PYANTTKYTYDYDGDGQLQSVAVNDRPTWRYSYDLNGNLHLLNPGNSVRLMPLRYDLRDR |
| NOV7s | PYANTTKYTYDYDGDGQLQSVAVNDRPTWRYSYDLNGNLHLLNPGNSVRLMPLRYDLRDR |
| NOV7t | ------------------------------------------------------------ |
| NOV7u | PYANTTKYTYDYDGDGQLQSVAVNDRPTWRYSYDLNGNLHLLNPGNSVRLMPLRYDLPDR |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | YANTTKYTYDYDGDGQLQSVPAVNDRPTWRYSYDLNGNLHLLNPGNSVRLMPLRYDLRDR |
| NOV7ac | PYANTTKYTYDYDGDGQLQSVAVNDRPTWRYSYDLNGNLHLLNPGNSVRLMPLRYDLRDR |
| NOV7a | ITRLGDVQYKIDDDGYLCQRGSDIFEYNSKGLLTRAYNKASGwSVQYRYDGVGRRASYKT |
| NOV7b | ITRLGDVQYKIDDDGYLCQRGSDIFEYNSKGLLTRAYNKASGwSVQYRYDGVGRRAsYKT |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | ITRLGDVQYKIDDDGYLCQRGSDIFEYNSKGLLTRAYNKASGWSVQYRYDGVGRRASYKT |
| NOV7m | ------------------------------------------------------------ |
| NOV7n | ------------------------------------------------------------ |
| NOV7o | NTTKYTYDYDGDGQLQSVAVNDRPTWRYSYDLNGNLHLLNPGNSVRLMPLRYDLRDRITR |
| NOV7p | ------------------------------------------------------------ |
| NOV7q | DYTMWKNVGKEPAPFNLYMFKSNNPLSSELDLKNYVTDVKSWLVMFGFQLSNIIPGFPRA |
| NOV7r | ITRLGDVQYKIDDDGYLCQRGSDIFEYNSKGLLTRAYNKASGWSVQYRYDGVGRRASYKT |
| NOV7s | ITRLGDVQYKIDDDGYLCQRGSDIFEYNSKGLLTRAYNKASGWSVQYRYDGVGRRASYKT |
| NOV7t | ------------------------------------------------------------ |
| NOV7u | ITRLGDVQYKIDDDGYLCQRGSDIFEYNSKGLLTRAYNKASGWSVQYRYDGVGRRASYKT |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | ITRLGDVQYKIDDDGYLCQRGSDIFEYNSKGLLTRAYNKASGWSVQYRYDGVGRRASYKT |
| NOV7ac | ITRLGDVQYKIDDDGYLCQRGSDIFEYNSKGLLTRAYNKASGWSVQYRYDGVGRRASYKT |
| NOV7a | NLGHHLQYFYSDLHNPTRITHVYNHSNSEITSLYYDLQGHLFAMESSSGEEYYVASDNTG |
| NOV7b | NLGHHLQYFYSDLHNPTRITHVYNHSNSEITSLYYDLQGHLFAMESSSGEEYYVASDNTG |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | NLGHHLQYFYSDLHNPTRITHVYNHSNSEITSLYYDLQGHLFAMESSSGEEYYVASDNTG |
| NOV7m | ------------------------------------------------------------ |
| NOV7n | ------------------------------------------------------------ |
| NOV7o | LGDVQYKIDDDGYLCQRGSDIFEYNSKGLLTPAYNKASGWSVQYRYDGVGRRASYKTNLG |
| NOV7p | ------------------------------------------------------------ |
| NOV7q | KMYFVPPPYELSESQASENGQLITGVQQTTERHNQAFMALEGQVITKKLHASIREKAGHW |
| NOV7r | NLGHHLQYFYSDLHNPTRITHVYNHSNSEITSLYYDLQGHLFAMESSSGEEYYVASDNTG |
| NOV7s | NLGHHLQYFYSDLHNPTRITHVYNHSNSEITSLYYDLQGHLFAMESSSGEEYYVASDNTG |
| NOV7t | ------------------------------------------------------------ |
| NOV7u | NLGHHLQYFYSDLHNPTRITHVYNHSNSEITSLYYDLQGHLFAMESSSGEEYYVASDNTG |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | NLGHHLQYFYSDLHNPTRITHVYNHSNSEITSLYYDLQGHLFAMESSSGEEYYVASDNTG |
| NOV7ac | NLGHHLQYFYSDLHNPTRITHVYNHSNSEITSLYYDLQGHLFAMESSSGEEYYVASDNTG |
| NOV7a | TPLAVFSINGLMIKQLQYTAYGEIYYDSNPDFQMVIGFHGGLYDPLTKLVHFTQRDYDVL |
| NOV7b | TPLAVFSINGLMIKQLQYTAYGEIYYDSNPDFQMVIGFHGGLYDPLTKLVHFTQRDYDVL |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | TPLAVFSINGLMIKQLQYTAYGEIYYDSNPDFQMVIGFHGGLYDPLTKLVHFTQRDYDVL |
| NOV7m | ------------------------------------------------------------ |
| NOV7n | ------------------------------------------------------------ |
| NOV7o | HHLQYFYSDLHNPTRITHVYNHSNSEITSLYYDLQGHLFAMESSSGEEYYVASDNTGTPL |
| NOV7p | ------------------------------------------------------------ |
| NOV7q | FATTTPIIGKGIMFAIKEGRVTTGVSSIASEDSRKVASVLNNAYYLDKMHYSIEGKDTHY |
| NOV7r | TPLAVFSINGLMIKQLQYTAYGEIYYDSNPDFQMVIGFHGGLYDPLTKLVHFTQRDYDVL |
| NOV7s | TPLAVFSINGLMIKQLQYTAYGEIYYDSNPDFQMVIGFHGGLYDPLTKLVHFTQRDYDVL |
| NOV7t | ------------------------------------------------------------ |
| NOV7u | TPLAVFSINGLMIKQLQYTAYGEIYYDSNPDFQMVIGFHGGLYDPLTKLVHFTQRDYDVL |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | TPLAVFSINGLMIKQLQYTAYGEIYYDSNPDFQMVIGFHGGLYDPLTKLVHFTQRDYDVL |
| NOV7ac | TPLAVFSINGLMIKQLQYTAYGEIYYDSNPDFQMVIGFHGGLYDPLTKLVHFTQRDYDVL |
| NOV7a | AGRWTSPDYTMWKNVGKEPAPFNLYMFKSNNPLSSELDLKNYVTDVKSWLVMFGFQLSNI |
| NOV7b | AGRWTSPDYTMWKNVGKEPAPFNLYMFKSNNPLSSELDLKNYVTDVKSWLVMFGFQLSNI |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | AGRWTSPDYTMWKNVGKEPAPFNLYMFKSNNPLSSELDLKNYVTDVKSWLVMFGFQLSNI |
| NOV7m | ------------------------------------------------------------ |
| NOV7n | ------------------------------------------------------------ |
| NOV7o | AVFSINGLMIKQLQYTAYGEIYYDSNPDFQMVIGFHGGLYDPLTKLVHFTQRDYDVLAGR |
| NOV7p | ------------------------------------------------------------ |
| NOV7q | FVKIGSADGDLVTLGTTIGRKVLESGVNVTVSQPTLLVNGRTRRFTNIEFQYSTLLLSIR |
| NOV7r | AGRWTSPDYTMWKNVGKEPAPFNLYMFKSNNPLSSELDLKNYVTDVKSWLVMFGFQLSNI |
| NOV7s | AGRWTSPDYTMWKNVGKEPAPFNLYMFKSNNPLSSELDLKNYVTDVKSWLVMFGFQLSNI |
| NOV7t | ------------------------------------------------------------ |
| NOV7u | AGRWTSPDYTMWKNVGKEPAPFNLYMFKSNNPLSSELDLKNYVTDVKSWLVMFGFQLSNI |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | AGRWTSPDYTMWKNVGKEPAPFNLYMFKSNNPLSSELDLKNYVTDVKSWLVMFGFQLSNI |
| NOV7ac | AGRWTSPDYTNWKNVGKEPAPFNLYMFKSNNPLSSELDLKNYVTDVKSWLVMFGFQLSNI |
| NOV7a | IPGFPRAXMYFVPPPYELSESQASENGQLITGVQQTTERHNQAFMALEGQVITKKLHASI |
| NOV7b | IPGFPRAKMYFVPPPYELSESQASENGQLITGVQQTTERHNQAFMALEGQVITKKLHASI |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | IPGFPRAKMYFVPPPYELSESQASENGQLITGVQQTTERHNQAFMALEGQVITKKLHASI |
| NOV7m | ------------------------------------------------------------ |
| NOV7n | ------------------------------------------------------------ |
| NOV7o | WTSPDYTMWKNVGKEPAPFNLYMFKSNNPLSSELDLKNYVTDVKSWLVMFGFQLSNIIPG |
| NOV7p | ------------------------------------------------------------ |
| NOV7q | YGLTPDTLDEEKARVLDQARQRALGTAWAKEQQKARDGREGSRLWTEGEKQQLLSTGRVQ |
| NOV7r | IPGFPRAKMYFVPPPYELSESQASENGQLITGVQQTTERHNQAFMALEGQVITKKLHASI |
| NOV7s | IPGFPRAKMYFVPPPYELSESQASENGQLITGVQQTTERHNQAFMALEGQVITKKLHASI |
| NOV7t | ------------------------------------------------------------ |
| NOV7u | IPGFPRAXMYFVPPPYELSESQASENGQLITGVQQTTERHNQAFMALEGQVITKKLHASI |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | IPGFPRAKMYFVPPPYELSESQASENGQLITGAHQTTERHNQAFMALEGQVITKKLHASI |
| NOV7ac | IPGFPRAKNYFVPPPYELSESQASENGQLITGVQQTTERHNQAFMALEGQVITKKLHASI |
| NOV7a | REKAGHWFATTTPIIGKGIMFAIKEGRVTTGVSSIASEDSRKVASVLNNAYYLDKMHYSI |
| NOV7b | REKAGHWFATTTPIIGKGIMFAIKEGRVTTGVSSIASEDSRKVASVLNNAYYLDKMHYSI |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | REKAGHWFATTTPIIGKGIMFAIKEGRVTTGVSSIASEDSRKVASVLNNAYYLDKMHYSI |
| NOV7m | ------------------------------------------------------------ |
| NOV7n | ------------------------------------------------------------ |
| NOV7o | FPRAKMYFVPPPYELSESQASENGQLITGVQQTTERHNQAFMALEGQVITKKLHASIREK |
| NOV7p | ------------------------------------------------------------ |
| NOV7q | GYEGYYVLPVEQYPELADSSSNIQFLRQNEMGKR-------------------------- |
| NOV7r | REKAGHWFATTTPIIGKGIMFAIKEGRVTTGVSSIASEDSRKVASVLNNAYYLDKMHYSI |
| NOV7s | REKAGHWFATTTPIIGKGIMFAIKEGRVTTGVSSIASEDSRKVASVLNNAYYLDKMHYSI |
| NOV7t | ------------------------------------------------------------ |
| NOV7u | REKAGHWFATTTPIIGKGIMFAIKEGRVTTGVSSIASEDSRKVASVLNNAYYLDKMHYSI |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | REKAGHWFATTTPIIGKGIMFAIKEGRVTTGVSSIASEDSRKVASVLNNAYYLDKMHYSI |
| NOV7ac | REKAGHWFATTTPIIGKGIMFAIKEGRVTTGVSSIASEDSRKVASVLNNAYYLDKMHYSI |
| NOV7a | EGKDTHYFVKIGSADGDLVTLGTTIGRYVLESGVNVTVSQPTLLVNGRTRRFTNIEFQYS |
| NOV7b | EGKDTHYFVKIGSADGDLVTLGTTIGRKVLESGVNVTVSQPTLLVNGRTRRFTNIEFQYS |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| Nov7l | EGKDTHYFVKIGSADGDLVTLGTTIGRKVLESGVNVTVSQPTLLVNGRTRRFTNIEFQYS |
| NOV7m | ------------------------------------------------------------ |
| NOV7n | ------------------------------------------------------------ |
| NOV7o | AGHWFATTTPITGKGIMFAIKEGRVTTGVSSIASEDSRKVASVLNNAYYLDKMHYSIEGK |
| NOV7p | ------------------------------------------------------------ |
| NOV7q | ------------------------------------------------------------ |
| NOV7r | EGKDTHYFVKIGSADGDLVTLGTTIGRKVLESGVNVTVSQPTLLVNGRTRRFTNIEFQYS |
| NOV7s | EGKDTHYFVKIGSADGDLVTLGTTIGRKVLESGVNVTVSQPTLLVNGRTRRFTNIEFQYS |
| NOV7t | ------------------------------------------------------------ |
| NOV7u | EGKDTHYFVKIGSADGDLVTLGTTIGRKVLESGVNVTVSQPTLLVNGRTRRFTNIEFQYS |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | EGKDTHYFVKIGSADGDLVTLGTTIGRKVLESGVNVTVSQPTLLVNGRTRRFTNIEFQYS |
| NOV7ac | EGKDTHYFVKIGSADGDLVTLGTTIGRKVLESGVNVTVSQPTLLVNGRTRRFTNIEFQYS |
| NOV7a | TLLLSIRYGLTPDTLDEEKARVLDQARQRALGTAWAKEQQKARDGREGSRLWTEGEKQQL |
| NOV7b | TLLLSIRYGLTPDTLDEEKARVLDQARQRALGTAWAKEQQKARDGREGSRLWTEGEKQQL |
| NOV7c | ------------------------------------------------------------ |
| NOV7d | ------------------------------------------------------------ |
| NOV7e | ------------------------------------------------------------ |
| Nov7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | TLLLSIRYGLTPDTLDEEKARVLDQARQRALGTAWAKEQQKARDGREGSRLWTEGEKQQL |
| NOV7m | ------------------------------------------------------------ |
| NOV7n | ------------------------------------------------------------ |
| NOV7o | DTHYFVKIGSADGDLVTLGTTIGRKVLESGVNVTVSQPTLLVNGRTRRFTNIEFQYSTLL |
| NOV7p | ------------------------------------------------------------ |
| NOV7q | ------------------------------------------------------------ |
| NOV7r | TLLLSIRYGLTPDTLDEEKARVLDQARQRALGTAWAKEQQKARDGREGSRLWTEGEKQQL |
| NOV7s | TLLLSIRYGLTPDTLDEEKARVLDQARQRALGTAWAKEQQKARDGREGSRLWTEGEKQQL |
| NOV7t | ------------------------------------------------------------ |
| NOV7u | TLLLSIRYGLTPDTLDEEKARVLDQARQRALGTAWAKEQQKARDGREGSRLWTEGEKQQL |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | TLLLSIRYGLTPDTLDEEKARVLDQARQRALGTAWAKEQQKAPDGREGSRLWTEGEKQQL |
| NOV7ac | TLLLSIRYGLTPDTLDEEKARVLDQARQRALGTAWAKEQQKARDGREGSRLWTEGEKQQL |
| NOV7a | LSTGRVQGYEGYYVLPVEQYPELADSSSNIQFLRQNEMGKR------------------- |
| NOV7b | LSTGRVQGYEGYYVLPVEQYPELADSSSNIQFLRQNEMGKR------------------- |
| NOV7c | ------------------------------------------------------------ |
| NOV7d | ------------------------------------------------------------ |
| NOV7e | ------------------------------------------------------------ |
| NOV7f | ------------------------------------------------------------ |
| NOV7g | ------------------------------------------------------------ |
| NOV7h | ------------------------------------------------------------ |
| NOV7i | ------------------------------------------------------------ |
| NOV7j | ------------------------------------------------------------ |
| NOV7k | ------------------------------------------------------------ |
| NOV7l | LSTGRVQGYEGYYVLPVEQYPELADSSSNIQFLRQNEMGKR------------------- |
| NOV7m | ------------------------------------------------------------ |
| NOV7o | LSIRYGLTPDTLDEEKARVLDQARQRALGTAWAKEQQKARDGREGSRLWTEGEKQQLLST |
| NOV7p | ------------------------------------------------------------ |
| NOV7q | ------------------------------------------------------------ |
| NOV7r | LSTGRVQGYEGYYVLPVEQYPELADSSSNIQFLRQNEMGKR------------------- |
| NOV7s | LSTGRVQGYEGYYVLPVEQYPELADSSSNIQFLRQNEMGKR------------------- |
| NOV7t | ------------------------------------------------------------ |
| NOV7u | LSTGRVQGYEGYYVLPVEQYPELADSSSNIQFLRQNEMGKR------------------- |
| NOV7v | ------------------------------------------------------------ |
| NOV7w | ------------------------------------------------------------ |
| NOV7x | ------------------------------------------------------------ |
| NOV7y | ------------------------------------------------------------ |
| NOV7z | ------------------------------------------------------------ |
| NOV7aa | ------------------------------------------------------------ |
| NOV7ab | LSTGRVQGYEGYYVLPVEQYPELADSSSNIQFLRQNENGKR------------------- |
| NOV7ac | LSTGRVQGYEGYYVLPVEQYPELADSSSNIQFLRQNEMGKR------------------- |
| NOV7a | -------------------------------------- |
| NOV7b | -------------------------------------- |
| NOV7c | -------------------------------------- |
| NOV7d | -------------------------------------- |
| NOV7e | -------------------------------------- |
| NOV7f | -------------------------------------- |
| NOV7g | -------------------------------------- |
| NOV7h | -------------------------------------- |
| NOV7i | -------------------------------------- |
| NOV7j | -------------------------------------- |
| NOV7k | -------------------------------------- |
| NOV7l | -------------------------------------- |
| NOV7m | -------------------------------------- |
| NOV7n | -------------------------------------- |
| NOV7o | GRVQGYEGYYVLPVEQYPELADSSSNIQFLRQNEMGKR |
| NOV7p | -------------------------------------- |
| NOV7q | -------------------------------------- |
| NOV7r | -------------------------------------- |
| NOV7s | -------------------------------------- |
| NOV7t | -------------------------------------- |
| NOV7u | -------------------------------------- |
| NOV7v | -------------------------------------- |
| NOV7w | -------------------------------------- |
| NOV7x | -------------------------------------- |
| NOV7y | -------------------------------------- |
| NOV7z | -------------------------------------- |
| NOV7aa | -------------------------------------- |
| NOV7ab | -------------------------------------- |
| NOV7ac | -------------------------------------- |
| NOV7a | (SEQ ID NO: 114) |
| NOV7b | (SEQ ID NO: 116) |
| NOV7c | (SEQ ID NO: 118) |
| NOV7d | (SEQ ID NO: 120) |
| NOV7e | (SEQ ID NO: 122) |
| NOV7f | (SEQ ID NO: 124) |
| NOV7g | (SEQ ID NO: 126) |
| NOV7h | (SEQ ID NO: 128) |
| NOV7i | (SEQ ID NO: 130) |
| NOV7j | (SEQ ID NO: 132) |
| NOV7k | (SEQ ID NO: 134) |
| NOV7l | (SEQ ID NO: 136) |
| NOV7m | (SEQ ID NO: 138) |
| NOV7n | (SEQ ID NO: 140) |
| NOV7o | (SEQ ID NO: 142) |
| NOV7p | (SEQ ID NO: 144) |
| NOV7q | (SEQ ID NO: 146) |
| NOV7r | (SEQ ID NO: 148) |
| NOV7s | (SEQ ID NO: 150) |
| NOV7t | (SEQ ID NO: 152) |
| NOV7u | (SEQ ID NO: 154) |
| NOV7v | (SEQ ID NO: 156) |
| NOV7w | (SEQ ID NO: 158) |
| NOV7x | (SEQ ID NO: 160) |
| NOV7y | (SEQ ID NO: 162) |
| NOV7z | (SEQ ID NO: 164) |
| NOV7aa | (SEQ ID NO: 166) |
| NOV7ab | (SEQ ID NO: 168) |
| NOV7ac | (SEQ ID NO: 170) |

Further analysis of the NOV7a protein yielded the following properties shown in Table 7C.

TABLE 7C
Protein Sequence Properties NOV7a

| | |
|---|---|
| SignalP analysis: | No Known Signal Sequence Predicted |
| PSORT II analysis: | |

| PSG: | a new signal peptide prediction method | |
|---|---|---|
| | N-region: | length 9; pos. chg 4; neg. chg 2 |
| | H-region: | length 3; peak value −8.37 |
| | PSG score: | −12.77 |

| | |
|---|---|
| GvH: | von Heijne's method for signal seq. recognition |
| | GvH score (threshold: −2.1): −14.20 |
| | possible cleavage site: between 32 and 33 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 2 | | |
| | Number of TMS(s) for threshold 0.5: 1 | | |
| | INTEGRAL | Likelihood = | Transmembrane 380-396 |
| | | −11.73 | |

| | |
|---|---|
| PERIPHERAL | Likelihood = 3.39 (at 982) |

| | |
|---|---|
| | ALOM score: −11.73 (number of TMSs: 1) |
| MTOP: | Prediction of membrane topology (Hartmann et al.) |
| | Center position for calculation: 387 |
| | Charge difference: −6.0 C(0.0)-N(6.0) |
| | N >= C: N-terminal side will be inside |

>>> membrane topology: type 2 (cytoplasmic tail 1 to 380)

| MITDISC: | discrimination of mitochondrial targeting seq | | | |
|---|---|---|---|---|
| | R content: | 0 | Hyd Moment(75): | 3.46 |
| | Hyd Moment(95): | 5.35 | G content: | 0 |
| | D/E content: | 2 | S/T content: | 0 |

| | |
|---|---|
| | Score: −7.50 |
| Gavel: | prediction of cleavage sites for mitochondrial preseq |
| | cleavage site motif not found |

NUCDISC: discrimination of nuclear localization signals

| | |
|---|---|
| | pat4: RRHR (3) at 6 |
| | pat7: PRERRSI (5) at 483 |
| | bipartite: RKEQIKGKVTIFGRKLR at 1712 |
| | content of basic residues: 9.7% |
| | NLS Score: 0.64 |
| KDEL: | ER retention motif in the C-terminus: none |

ER Membrane Retention Signals:

KKXX-like motif in the C-terminus: EMGK

SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: found

LL at 179
LL at 200
LL at 224
LL at 351

checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 70.6 |
| COIL: | Lupas's algorithm to detect coiled-coil regions |
| | total: 0 residues• |

Final Results (k = 9/23):

```
43.5%: nuclear
21.7%: mitochondrial
13.0%: cytoplasmic
  8.7%: Golgi
  4.3%: plasma membrane
  4.3%: extracellular, including cell wall
  4.3%: peroxisomal
```

>> prediction for CG50426-17 is nuc (k = 23)

A search of the NOV7a protein against the Geneseq database, a proprietary database that contains sequences published in patents and patent publication, yielded several homologous proteins shown in Table 7D.

TABLE 7D
Geneseq Results for NOV7a

| | | NOV7a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| AAU08681 | Human FCTR3f polypeptide | 1 . . . 2662 | 2620/2728 (96%) | 0.0 |
| | sequence - Homo sapiens, 2724 aa. | 1 . . . 2724 | 2628/2728 (96%) | |
| | [WO200166747-A2, 13-SEP-2001] | | | |
| AAU08680 | Human FCTR3b polypeptide | 1 . . . 2662 | 2620/2737 (95%) | 0.0 |
| | sequence - Homo sapiens, 2733 aa. | 1 . . . 2733 | 2628/2737 (95%) | |
| | [WO200166747-A2, 13-SEP-2001] | | | |
| ABP53587 | Human NOV15b protein SEQ ID | 1 . . . 2662 | 1780/2799 (63%) | 0.0 |
| | NO: 38 - Homo sapiens, 2721 aa. | 1 . . . 2721 | 2156/2799 (76%) | |
| | [WO200262999-A2, 15-AUG-2002] | | | |
| ABP53586 | Human NOV15a protein SEQ ID | 1 . . . 2662 | 1779/2803 (63%) | 0.0 |
| | NO: 36 - Homo sapiens, 2725 aa. | 1 . . . 2725 | 2155/2803 (76%) | |
| | [WO200262999-A2, 15-AUG-2002] | | | |
| ABP53589 | Human NOV15d protein SEQ ID | 239 . . . 2662 | 1689/2546 (66%) | 0.0 |
| | NO: 42 - Homo sapiens, 2613 aa. | 80 . . . 2613 | 2035/2546 (79%) | |
| | [WO200262999-A2, 15-AUG-2002] | | | |

In a BLAST search of public sequence databases, the NOV7a protein was found to have homology to the proteins shown in the BLASTP data in Table 7E.

TABLE 7E
Public BLASTP Results for NOV7a

| | | NOV7a | | |
|---|---|---|---|---|
| Protein | | Residues/ | Identities/ | |
| Accession | | Match | Similarities for the | Expect |
| Number | Protein/Organism/Length | Residues | Matched Portion | Value |
| CAC88676 | Sequence 12 from Patent | 1 . . . 2662 | 2620/2728 (96%) | 0.0 |
| | WO0166747 - Homo sapiens | 1 . . . 2724 | 2628/2728 (96%) | |
| | (Human), 2724 aa. | | | |
| CAC88675 | Sequence 7 from Patent | 1 . . . 2662 | 2620/2737 (95%) | 0.0 |
| | WO0166747 - Homo sapiens | 1 . . . 2733 | 2628/2737 (95%) | |
| | (Human), 2733 aa. | | | |
| Q9WTS5 | Ten-m2 - Mus musculus (Mouse), | 1 . . . 2662 | 2582/2768 (93%) | 0.0 |
| | 2764 aa. | 1 . . . 2764 | 2611/2768 (94%) | |
| Q9R1K2 | Neurestin alpha - Rattus | 1 . . . 2662 | 2574/2769 (92%) | 0.0 |
| | norvegicus (Rat), 2765 aa. | 1 . . . 2765 | 2610/2769 (93%) | |
| Q9ULU2 | KIAA1127 protein - Homo sapiens | 590 . . . 2662 | 2029/2148 (94%) | 0.0 |
| | (Human), 2144 aa (fragment). | 1 . . . 2144 | 2037/2148 (94%) | |

PFam analysis predicts that the NOV7a protein contains the domains shown in the Table 7F.

TABLE 7F
Domain Analysis of NOV7a

| | | Identities/ | |
|---|---|---|---|
| | | Similarities | |
| Pfam | NOV7a | for the | Expect |
| Domain | Match Region | Matched Region | Value |
| EGF | 599 . . . 626 | 10/47 (21%) | 0.44 |
| | | 20/47 (43%) | |
| EGF | 631 . . . 658 | 13/47 (28%) | 0.0037 |
| | | 20/47 (43%) | |
| EGF | 698 . . . 728 | 9/47 (19%) | 0.019 |
| | | 24/47 (51%) | |
| NHL | 1302 . . . 1337 | 12/36 (33%) | 0.36 |
| | | 27/36 (75%) | |
| NHL | 1431 . . . 1458 | 12/29 (41%) | 0.00074 |
| | | 23/29 (79%) | |

### Example 8.

The NOV8 clone was analyzed, and the nucleotide and encoded polypeptide sequences are shown in Table 8A.

TABLE 8A
NOV8 Sequence Analysis

| | | |
|---|---|---|
| NOV8a, CG50646-04 | SEQ ID NO: 177 | 11158 bp |

| | | |
|---|---|---|
| DNA Sequence | ORF Start: ATG at 77 | ORF Stop: TAA at 10787 |

CAATTGGTCTAGGGTCTCCCCCATTGGAATATCCATCAGTGATGAGAAATACAACGTTTGTTGAGTTT
TCTCTAGCATGAGAAGAATTTGCGCGGCTTGCTGGGGTCTGGCGCTCGTTTCGGGCTGGGCGACCTTT
CAGCAGATGTCCCCGTCGCGCAATTTCAGCTTCCGCCTCTTCCCCGAGACCGCGCCCGGGGCCCCCGG
GAGTATCCCCGCGCCGCCCGCTCCTGGCGACGAAGCGGCGGGGAGCAGAGTGGAGCGGCTGGGCCAGG
CGTTCCGCGTGCGGCTGCTGCGGGAGCTCAGCGAGCGCCTGGAGCTTGTCTTCCTGGTGGATGATTCG
TCCAGCGTGGGCGAAGTCAACTTCCGCAGCGAGCTCATGTTCGTCCGCAAGCTGCTGTCCGACTTCCC
CGTGGTGCCCACGGCCACGCGCGTGGCCATCGTGACCTTCTCGTCCAAGAACTACGTGGTGCCGCGCG
TCGATTACATCTCCACCCGCCGCGCGCGCCAGCACAAGTGCGCGCTGCTCCTCCAAGAGATCCCTGCC
ATCTCCTACCGAGGTGGCGGCACCTACACCAAGGGCGCCTTCCAGCAAGCCGCGCAAATTCTTCTTCA
TGCTAGAGAAAACTCAACAAAAGTTGTATTTCTCATCACTGATGGATATTCCAATGGGGGAGACCCTA
GACCAATTGCAGCGTCACTGCGAGATTCAGGAGTGGAGATCTTCACTTTTGGCATATGGCAAGGGAAC
ATTCGAGAGCTGAATGACATGGCTTCCACCCCAAAGGAGGAGCACTGTTACCTGCTACACAGTTTTGA
AGAATTTGAGGCTTTAGTCGCCCTCTGTCATATGTTATTTGTAGATCTACCTTCTGGGAGTTTTATTC
AAGATGATATGGTCCACTGCTCATATCTTTGTGATGAAGGCAAGGACTGCTGTGACCGAATGGGAAGC
TGCAAATGTGGGAAACACACAGGCCATTTTGAGTGCATCTGTGAAAAGGGGTATAACGGGAAAGGTCT
GCAGTATGACTGCACAGTTTGCCCATCGGGGACATACAAACCTGAAGGCTCACCAGGAGGAATCAGCA
TGCAGAGAGGGATACAGGGCATCTGGCCAGACCTGTGAAGTTGTCCACTGCCCTGCCCTGAAGCCTCC
CGAAAATGGTTACTTTATCCAAAACACTTGCAACAACCACTTCAATGCAGCCTGTGGGGTCCGATGTC
ACCCTGGATTTGATCTTGTGGGAAGCAGCATCATCTTATGTCTACCCAATGGTTTGTGGTCCGGTTCA
GAGAGCTACTGCAGAGTAAGAACATGTCCTCATCTCCGCCAGCCGAAACATGGCCACATCAGCTGTTC
TACAAGGGAAATGTTATATAAGACAACATGTTTGGTTGCCTGTGATGAAGGGTACAGGCTAGAAGGCA
GTGATAAGCTTACTTGTCAAGGAAACAGCCAGTGGGATGGGCCAGAACCCCGGTGTGTGGAGCGCCAC
TGTTCCACCTTTCAGATGCCCAAAGATGTCATCATATCCCCCCACAACTGTGGCAAGCAGCCAGCCAA
ATTTGGGACGATCTGCTATGTAAGTTGCCGCCAAGGGTTCATTTTATCTGGAGTCAAAGAAATGCTGA
GATGTACCACTTCTGGAAAATGGAATGTCGGAGTTCAGGCAGCTGTGTGTAAAGACGTGGAGGCTCCT
CAAATCAACTGTCCTAAGGACATAGAGGCTAAGACTCTGGAACAGCAAGATTCTGCCAATGTTACCTG
GCAGATTCCAACAGCTAAAGACAACTCTGGTGAAAAGGTGTCAGTCCACGTTCATCCAGCTTTCACCC
CACCTTACCTTTTCCCAATTGGAGATGTTGCTATCGTATACACGGCAACTGACCTATCCGGCAACCAG
GCCAGCTGCATTTTCCATATCAAGGTTATTGATGCAGAACCACCTGTCATAGACTGGTGCAGATCTCC
ACCTCCCGTCCAGGTCTCGGAGAAGGTACATGCCGCAAGCTGGGATGAGCCTCAGTTCTCAGACAACT
CAGGTGCTGAATTGGTCATTACCAGAAGTCATACACAAGGAGACCTTTTCCCTCAAGGGGAGACTATA
GTACAGTATACAGCCACTGACCCCTCAGGCAATAACAGGACATGTGATATCCATATTGTCATAAAAGG
TTCTCCCTGTGAAATTCCATTCACACCTGTAAATGGGGATTTTATATGCACTCCAGATAATACTGGAG
TCAACTGTACATTAACTTGCTTGGAGGGCTATGATTTCACAGAAGGGTCTACTGACAAGTATTATTGT
GCTTATGAAGATGGCGTCTGGAAACCAACATATACCACTGAATGGCCAGACTGTGCCAGTAAGCGTTT
TGCAAACCACGGGTTCAAGTCCTTTGAGATGTTCTACAAAGCAGCTCGTTGTGATGACACAGATCTGA
TGAAGAAGTTTTCTGAAGCATTTGAGACGACCCTGGGAAAAATGGTCCCATCATTTTGTAGTGATGCA
GAGGACATTGACTGCAGACTGGAGGAGAACCTGACCAAAAAATATTGCCTAGAATATAATTATGACTA
TGAAAATGGCTTTGCAATTGGTCCAGGTGGCTGGGGTGCAGCTAATAGGCTGGATTACTCTTACGATG
ACTTCCTGGACACTGTGCAAGAAACAGCCACAAGCATCGGCAATGCCAAGTCCTCACGGATTAAAAGA
AGTGCCCCATTATCTGACTATAAAATTAAGTTAATTTTTAACATCACAGCTAGTGTGCCATTACCCGA
TGAAAGAAATGATACCCTTGAATGGGAAAATCAGCAACGACTCCTTCAGACATTGGAAACTATCACAA
GCCGACAGCAATTCATTAGAAACAAAAAAGGCTTCCCCCTTCTGCAGACCAGGCTCAGTGCTGAGAGG
GGATCGGATCCTATCAAGATGAAGAAGGGCAACTTGAGTGCAAGCTTTGCCCCTCTGGGATGTACACG
GAATATATCCATTCAAGAAACATCTCTGATTGTAAAGCTCAGTGTAAACAAGGCACCTACTCATACAG
TGGACTTGAGACTTGTGAATCGTGTCCACTGGGCACTTATCAGCCAAAATTTGGTTCCCGGAGCTGCC
TCTCGTGTCCAGAAAACACCTCAACTGTGAAAAGAGGAGCCGTGAACATTTCTGCATGTGGAGTTCCT
TGTCCAGAAGGAAAATTCTCGCGTTCTGGGTTAATGCCCTGTCACCCATGTCCTCGTGACTATTACCA
ACCTAATGCAGGGAAGGCCTTCTGCCTGGCCTGTCCCTTTTATGGAACTACCCCATTCGCTGGTTCCA
GATCCATCACAGAATGTTCAAGTTTTAGTTCAACTTTCTCAGCGGCAGAGGAAAGTGTGGTGCCCCCT
GCCTCTCTTGGACATATTAAAAAGAGGCATGAAATCAGCAGTCAGGCAAGTCATGAATGCTTCTTTAA
ATACAGGTTTAAAGTGTGAAACAGACATCGATGAGTGCAGCCCACTGCCTTGCCTCAACAATGGAGTT
TGTAAAGACCTAGTTGGGGAATTCATTTGTGAGTGCCCATCAGGTTACACAGGTAAGCACTGTGAATT
GAACATCAATGAATGTCAGTCTAATCCATGTAGAAATCAGGCCACCTGTGTGGATGAATTAAATTCAT
ACAGTTGTAAATGTCAGCCAGGATTTTCAGGCAAAAGGTGTGAAACAGGTATGTATCAACTCAGTGTT
ATTAATAACCTTAATAATGCAGTCTGTGAAGACCAGGTTGGGGGATTCTTGTGCAAATGCCCACCTGG
CCTGTAAAGACGGTGCCAATAGCTTCAGGTGCCTGTGTGCAGCTGGCTTCACAGGATCACACTGTGAA
TTGAACATCAATGAATGTCAGTCTAATCCATGTAGAAATCAGGCCACCTGTGTGGATGAATTAAATTC
ACCTGGATTTTGAAGTTTCTGGCATCTATGGATATGTCATGCTAGATGGCATGCTCCCATCTCTCCAT
GCTCTAACCTGTACCTTCTGGATGAAATCCTCTGACGACATGAACTATGGAACACCAATCTCCTATGC
AGTTGATAACGGCAGCGACAATACCTTGCTCCTGACTGATTATAACGGGTGGGTTCTTTATGTGAATG
ACAAGTACTGGTGGAGCCTGGAGGGTCTATATAAATGGGGAATTATCTGACGGTGGTACTGGCCTCTC
CATTGGCAAAGCCATACCTGGTGGCGGTGCATTAGTTCTTGGGCAAGAGCAAGACAAAAAAGGAGAGG
GGTTCAACCCGGCTGAGTCTTTTGTGGGCTCCATAAGCCAGCTCAACCTCTGGGACTATGTCCTGTCT
CCACAGCAGGTGAAGTCACTGGCTACCTCCTGCCCAGAGGAACTCAGTAAAGGAAACGTGTTAGCATG
GTCAATCTGTTCTGTGAACCAGGCTTCCAGCTGGTCGGGAACCCTGTGCAGTACTGTCTGAATCAAGG
ACAGTGGACACAACCACTCCCCCACTGTGAACGCATTCGCTGTGGGGTGCCACCTCCTTTGGAGAATG
CTATTGGGTGACTCAAGGATGTTCTGTACAGATAATGGGAGCTGGAACGGCGTTTCACCATCCTGCTT
AGATGTCGATGAGTGTGCAGTTGGATCAGATTGTAGTGAGCATGCTTCTTGCCTGAACGTAGATGGAT
CCTACATATGTTCATGTGTCCCACCGTACACAGGAGATGGGAAAAACTGTGCAGAACCTATAAAATGT
AAGGCTCCAGGAAATCCGGAAAATGGCCACTCCTCAGGTGAGATTTATACAGTAGGTGCCGAAGTCAC
ATTTTCGTGTCAGGAAGGATACCAGTTGATGGGAGTAACCAAAATCACATGTTTGGAGTCTGGAGAAT
GGAATCATCTAATACCATATTGTAAAGCTGTTTCATGTGGTAAACCGGCTATTCCAGAAAATGGTTGC
ATTGAGGAGTTAGCATTTACTTTTGGCAGCAAAGTGACATATAGGTGTAATAAAGGATATACTCTGGC
CGGTGATAAAGAATCATCCTGTCTTGCTAACAGTTCTTGGAGTCATTCCCCTCCTGTGTGTGAACCAG
TGAAGTGTTCTAGTCCGGAAAATATAAATAATGGAAAATATATTTTGAGTGGGCTTACCTACCTTTCT
ACTGCATCATATTCATGCGATACAGGATACAGCTTACAGGGCCCTTCCATTATTGAATGCACGGCTTC
TGGCATCTGGGACAGAGCGCCACCTGCCTGTCACCTCGTCTTCTGTGGAGAACCACCTGCCATCAAAG
ATGCTGTCATTACGGGGAATAACTTCACTTTCAGGAACACCGTCACTTACACTTGCAAAGAAGGCTAT
ACTCTTGCTGGTCTTGACACCATTGAATGCCTGGCCGACGGCAAGTGGAGTAGAAGTGACCAGCAGTG
CCTGGCTGTCTCCTGTGATGAGCCACCCATTGTGGACCACGCCTCTCCAGAGACTGCCCATCGGCTCT
TTGGAGACATTGCATTCTACTACTGCTCTGATGGTTACAGCCTAGCAGACAATTCCCAGCTTCTCTGC
AATGCCCAGGGCAAGTGGGTACCCCCAGAAGGTCAAGACATGCCCCGTTGTATAGCTCATTTCTGTGA
AAAACCTCCATCGGTTTCCTATAGCATCTTGGAATCTGTGAGCAAAGCAAAATTTGCAGCTGGCTCAG
TTGTGAGCTTTAAATGCATGGAAGGCTTTGTACTGAACACCTCAGCAAAGATTGAATGTATGAGAGGT
GGGCAGTGGAACCCTTCCCCCATGTCCATCCAGTGCATCCCTGTGCGGTGTGGAGAGCCACCAAGCAT
CATGAATGGCTATGCAAGTGGATCAAACTACAGTTTTGGAGCCATGGTGGCTTACAGCTGCAACAAGG
GGTTCTACATCAAAGGGGAAAAGAAGAGCACCTGCGAAGCCACAGGGCAGTGGAGTAGTCCTATACCG
ACGTGCCACCCGGTATCTTGTGGTGAACCACCTAAGGTTGAGAATGGCTTTCTGGAGCATACAACTGG
CAGGATCTTTGAGAGTGAAGTGAGGTATCAGTGTAACCCGGGCTATAAGTCAGTCGGAAGTCCTGTAT
TTGTCTGCCAAGCCAATCGCCACTGGCACAGTGAATCCCCTCTGATGTGTGTTCCTCTCGACTGTGGA
AAACCTCCCCCGATCCAGAATGGCTTCATGAAAGGAGAAAACTTTGAAGTAGGGTCCAAGGTTCAGTT
TTTCTGTAATGAGGGTTATGAGCTTGTTGGTGACAGTTCTTGGACATGTCAGAAATCTGGCAAATGGA
ATAAGAAGTCAAATCCAAAGTGCATGCCTGCCAAGTGCCCAGAGCCGCCCCTCTTGGAAAACCAGCTA
GTATTAAAGGAGTTGACCACCGAGGTAGGAGTTGTGACATTTTCCTGTAAAGAAGGGCATGTCCTGCA
AGGCCCCTCTGTCCTGAAATGCTTGCCATCCCAGCAATGGAATGACTCTTTCCCTGTTTGTAAGATTG
TTCTTTGTACCCCACCTCCCCTAATTTCCTTTGGTGTCCCCATTCCTTCTTCTGCTCTTCATTTTGGA
AGTACTGTCAAGTATTCTTGTGTAGGTGGGTTTTTCCTAAGAGGAAATTCTACCACCCTCTGCCAACC
TGATGGCACCTGGAGCTCTCCACTGCCAGAATGTGTTCCAGTAGAATGTCCCCAACCTGAGGAAATCC
CCAATGGAATCATTGATGTGCAAGGCCTTGCCTATCTCAGCACAGCTCTCTATACCTGCAAGCCAGGC
TTTGAATTGGTGGGAAATACTACCACCCTTTGTGGAGAAAATGGTCACTGGCTTGGAGGAAAACCAAC
ATGTAAAGCCATTGAGTGCCTGAAACCCAAGGAGATTTTGAATGGCAAATTCTCTTACACGGACCTAC
ACTATGGACAGACCGTTACCTACTCTTGCAACCGAGGCTTTCGGCTCGAAGGTCCCAGTGCCTTGACC
TGTTTAGAGACAGGTGATTGGGATGTAGATGCCCCATCTTGCAATGCCATCCACTGTGATTCCCCACA
ACCCATTGAAAATGGTTTTGTAGAAGGTGCAGATTACAGCTATGGTGCCATAATCATCTACAGTTGCT
TCCCTGGGTTTCAGGTGGCTGGTCATGCCATGCAGACCTGTGAAGAGTCAGGATGGTCAAGTTCCATC
CCAACATGTATGCCAATAGACTGTGGCCTCCCTCCTCATATAGATTTTGGAGACTGTACTAAACTCAA
AGATGACCAGGGATATTTTGAGCAAGAAGACGACATGATGGAAGTTCCATATGTGACTCCTCACCCTC
CTTATCATTTGGGAGCAGTGGCTAAAACCTGGGAAAATACAAAGGAGTCTCCTGCTACACATTCATCA
AACTTTCTGTATGGTACCATGGTTTCATACACCTGTAATCCAGGATATGAACTTCTGGGGAACCCTGT
GCTGATCTGCCAGGAAGATGGAACTTGGAATGGCAGTGCACCATCCTGCATTTCAATTGAATGTGACT
TGCCTACTGCTCCTGAAAATGGCTTTTTGCGTTTTACAGAGACTAGCATGGGAAGTGCTGTGCAGTAT
AGCTGTAAACCTGGACACATTCTAGCAGGCTCTGACTTAAGGCTTTGTCTAGAGAATAGAAAGTGGAG
AAGGAAGCAACTACACATACCTGAGCACGTTGTACTATGAGTGTGACCCCGGATATGTGCTGAATGGC
CTGCAGTTCACCCCCAGTCTCAGCCAATGGCCAGGTGAGAGGAGACGAGTACACATTCCAAAAAGAGA
TTGAATACACTTGCAATGAAGGGTTCTTGCTTGAGGGAGCCAGGAGTCGGGTTTGTCTTGCCAATGGA
AGTTGGAGTGGAGCCACTCCCGACTGTGTGCCTGTCAGATGTGCCACCCCGCCACAACTGGCCAATGG
GGTGACGGAAGGCCTGGACTATGGCTTCATGAAGGAAGTAACATTCCACTGTCACGAGGGCTACATCT
TGCACGGTGCTCCAAAACTCACCTGTCAGTCAGATGGCAACTGGGATGCAGAGATTCCTCTCTGTAAA
CCAGTCAACTGTGGACCTCCTGAAGATCTTGCCCATGGTTTCCCTAATGGTTTTTCCTTTATTCATGG
GGGCCATATACAGTATCAGTGCTTTCCTGGTTATAAGCTCCATGGAAATTCATCAAGAAGGTGCCTCT
CCAATGGCTCCTGGAGTGGCAGCTCACCTTCCTGCCTGCCTTGCAGATGTTCCACACCAGTAATTGAA
TATGGAACTGTCAATGGGACAGATTTTGACTGTGGAAAGGCAGCCCGGATTCAGTGCTTCAAAGGCTT
GTGAACACACTTCTTGTGGTTCTCTTCCAATGATACCAAATGCGTTCATCAGTGAGACCAGCTCTTGG
AAGGAAAATGTGATAACTTACAGCTGCAGGTCTGGATATGTCATACAAGGCAGTTCAGATCTGATTTG
TACAGAGAAAGGGGTATGGAGCCAGCCTTATCCAGTCTGTGAGCCCTTGTCCTGTGGGTCCCCACCGT
CTGTCGCCAATGCAGTGGCAACTGGAGAGGCACACACCTATGAAAGTGAAGTGAAACTCAGATGTCTG
GAAGGTTATACGATGGATACAGATACAGATACATTCACCTGTCAGAAAGATGGTCGCTGGTTCCCTGA
GAGAATCTCCTGCAGTCCTAAAAAATGTCCTCTCCCGGAAAACATAACACATATACTTGTACATGGGG
ACGATTTCAGTGTGAATAGGCAAGTTTCTGTGTCATGTGCAGAAGGGTATACCTTTGAGGGAGTTAAC
ATATCAGTATGTCAGCTTGATGGAACCTGGGAGCCACCATTCTCCGATGAATCTTGCAGTCCAGTTTC
TTGTGGGAAACCTGAAAGTCCAGAACATGGATTTGTGGTTGGCAGTAAATACACCTTTGAAAGCACAA
TTATTTATCAGTGTGAGCCTGGCTATGAACTAGAGGGGAACAGGGAACGTGTCTGCCAGGAGAACAGA
CAGTGGAGTGGAGGGGTGGCAATATGCAAAGAGACCAGGTGTGAAACTCCACTTGAATTTCTCAATGG
GAAAGCTGACATTGAAAACAGGACGACTGGACCCAACGTGGTATATTCCTGCAACAGAGGCTACAGTC
TTGAAGGGCCATCTGAGGCACACTGCACAGAAAATGGAACCTGGAGCCACCCAGTCCCTCTCTGCAAA
TGATCAGAATGTGTCCATCAAATGTAGGGAAGGTTTTCTGCTGCAGGGCCACGGCATCATTACCTGCA
GTAGAAAATGCAATTGCTCGAGGCGTACATTATCAATATGGAGACATGATCACCTACTCATGTTACAG
TGGATACATGTTGGAGGGTTTCCTGAGGAGTGTTTGTTTAGAAAATGGAACATGGACATCACCTCCTA
TTTGCAGAGCTGTCTGTCGATTTCCATGTCAGAATGGGGGCATCTGCCAACGCCCAAATGCTTGTTCC
TGTCCAGAGGGCTGGATGGGGCGCCTCTGTGAAGAACCAATCTGCATTCTTCCCTGTCTGAACGGAGG
TCGCTGTGTGGCCCCTTACCAGTGTGACTGCCCGCCTGGCTGGACGGGGTCTCGCTGTCATACAGCTG
TTTGCCAGTCTCCCTGCTTAAATGGTGGAAAATGTGTAAGACCAAACCGATGTCACTGTCTTTCTTCT
TGGACGGGACATAACTGTTCCAGGAAAAGGAGGACTGGGTTTTAACCACTGCACGACCATCTGGCTCT
CCCAAAAGCAGGATCATCTCTCCTCGGTAGTGCCTGGGCATCCTGGAACTTATGCAAAGAAAGTCCAA
CATGGTGCTGGGTCTTGTTTAGTAAACTTGTTACTTGGGGTTACTTTTTTTATTTTGTGATATATTTT
GTTATTCCTTGTGACATACTTTCTTACATGTTTCCATTTTTAAATATGCCTGTATTTTCTATATAAAA
ATTATATTAAATAGATGCTGCTCTACCCTCACAAAATGTACATATTCTGCTGTCTATTGGGAAAGTTC
CTGGTACACATTTTTATTCAGTTACTTAAAATGATTTTTCCATTAAAGTATATTTTGCTACTAAATAA
AAAAAA

NOV8a CG50646-04

| | | | |
|---|---|---|---|
| Protein sequence | SEQ ID NO: 178 | 357O aa | MW at 390012.5kD |

MRRICAACWGLALVSGWATFQQMSPSRNFSFRLFPETAPGAPGSIPAPPAPGDEAAGSRVERLGQAFR
VRLLRELSERLELVFLVDDSSSVGEVNFRSELMFVRKLLSDFPVVPTATRVAILVTFSSKNYVVPRVDY
ISTRRARQHKCALLLQEIPAISYRGGGTYTKGAFQQAAQILLHARENSTKVVFLITDGYSNGGDPRPI
AASLRDSGVEIFTFGIWQGNIRELNDMASTPKEEHCYLLHSFEEFEALVALCHMLFVDLPSGSFIQDD
MVHCSYLCDEGKDCCDRMGSCKCGKHTGHFECICEKGYNGKGLQYDCTVCPSGTYKPEGSPGGISSCI
PCPDENHTSPPGSTSPEDCVCREGYRASGQTCEVVHCPALKPPENGYFIQNTCNNHFNAACGVRCHPG
FDLVGSSIILCLPNGLWSGSESYCRVRTCPHLRQPKHGHISCSTREMLYKTTCLVACDEGYRLEGSDK
LTCQGNSQWDGPEPRCVERHCSTFQMPKDVIISPHNCGKQPAKFGTICYVSCRQGFILSGVKEMLRCT
TSGKWNVGVQAAVCKDVEAPQINCPKDIEAKTLEQQDSANVTWQIPTAKDNSGEKVSVHVHPAFTPPY
LFPIGDVAIVYTATDLSGNQASCIFHIKVIDAEPPVIDWCRSPPPVQVSEKVHAASSWEPQFSDNSGA
ELVITRSHTQGDLFPQGETIVQYTATDPSGNNRTCDIHIVIKGSPCEIPFTPVNGDFICTPDNTGVNC
TLTCLEGYDFTEGSTDKYYCAYEDGVWKPTYTTEWPDCASKRFANHGFKSFEMFYKAARCDDTDLMKK
FSEAFETTLGKMVPSFCSDAEDIDCRLEENLTKKYCLEYNYDYENGFAIGPGGWGAANRLDYSYDDFL
DTVQETATSIGNAKSSRIKRSAPLSDYKIKLIFNITASVPLPDERNDTLEWENQQRLLQTLETITNKL
KRTLNKDPMYSFQLASEILIADSNSLETKKASPFCRPGSVLRGRMCVNCPLGTYYNLEHFTCESCRIG
SYQDEEGQLECKLCPSGMYTEYIHSRNISDCKAQCKQGTYSYSGLETCESCPLGTYQPKFGSRSCLSC
PENTSTVKRGAVNISACGVPCPEGKFSRSGLMPCHPCPRDYYQPNAGKAFCLACPFYGTTPFAGSRSI
TECSSFSSTFSAAEESVVPPASLGHIKKRHEISSQASHECFFNPCHNSGTCQQLGRGYVCLCPLGYTG
LKCETDIDECSPLPCLNNGVCKDLVGEFICECPSGYTGKHCELNINECQSNPCRNQATCVDELNSYSC
KCQPGFSGKRCETGMYQLSVINNLNNAVCEDQVGGFLCKCPPGFLGTRCGKNVDECLSQPCKNGATCK
DGANSFRCLCAAGFTGSHCELNINECQSNPCRNQATCVDELNSYSCKCQPGFSGKRCETEQSTGFNLD
FEVSGIYGYVMLDGMLPSLHALTCTFWMKSSDDMNYGTPISYAVDNGSDNTLLLTDYNGWVLYVNGRE
KITNCPSVNDGRWHHIAITWTSTGGAWRVYINGELSDGGTGLSIGKAIPGGGALVLGQEQDKKGEGFN
PAESFVGSISQLNLWDYVLSPQQVKSLATSCPEELSKGNVLAWPDFLSGIVGKVKIDSKSIFCSDCPR
LGGSVPHLRTASEDLKPGSKVNLFCEPGFQLVGNPVQYCLNQGQWTQPLPHCERIRCGVPPPLENGFH
SADDFYAGSTVTYQCNNGYYLLGDSRMFCTDNGSWNGVSPSCLDVDECAVGSDCSEHASCLNVDGSYI
CSCVPPYTGDGKNCAEPIKCKAPGNPENGHSSGEIYTVGAEVTFSCQEGYQLMGVTKITCLESGEWNH
LIPYCKAVSCGKPAIPENGCIEELAFTFGSKVTYRCNKGYTLAGDKESSCLANSSWSHSPPVCEPVKC
SSPENINNGKYILSGLTYLSTASYSCDTGYSLQGPSIIECTASGIWDRAPPACHLVFCGEPPAIKDAV
ITGNNFTFRNTVTYTCKEGYTLAGLDTIECLADGKWSRSDQQCLAVSCDEPPIVDHASPETAHRLFGD
IAFYYCSDGYSLADNSQLLCNAQGKWVPPEGQDMPRCIAHFCEKPPSVSYSILESVSKAKFAAGSVVS
FKCMEGFVLNTSAKIECMRGGQWNPSPMSIQCIPVRCGEPPSIMNGYASGSNYSFGAMVAYSCNKGFY
IKGEKKSTCEATGQWSSPIPTCHPVSCGEPPKVENGFLEHTTGRIFESEVRYQCNPGYKSVGSPVFVC
QANRHWHSESPLMCVPLDCGKPPPIQNGFMKGENFEVGSKVQFFCNEGYELVGDSSWTCQKSGKWNKK
SNPKCMPAKCPEPPLLENQLVLKELTTEVGVVTFSCKEGHVLQGPSVLKCLPSQQWNDSFPVCKIVLC
TPPPLISFGVPIPSSALHFGSTVKYSCVGGFFLRGNSTTLCQPDGTWSSPLPECVPVECPQPEEIPNG
IIDVQGLAYLSTALYTCKPGFELVGNTTTLCGENGHWLGGKPTCKAIECLKPKEILNGKFSYTDLHYG
QTVTYSCNRGFRLEGPSALTCLETGDWDVDAPSCNAIHCDSPQPIENGFVEGADYSYGAIIIYSCFPG
FQVAGHAMQTCEESGWSSSIPTCMPIDCGLPPHIDFGDCTKLKDDQGYFEQEDDMMEVPYVTPHPPYH
LGAVAKTWENTKESPATHSSNFLYGTMVSYTCNPGYELLGNPVLICQEDGTWNGSAPSCISIECDLPT
APENGFLRFTETSMGSAVQYSCKPGHILAGSDLRLCLENRKWSGASPRCEAISCKKPNPVMNGSIKGS
NYTYLSTLYYECDPGYVLNGTERRTCQDDKNWDEDEPICIPVDCSSPPVSANGQVRGDEYTFQKEIEY
TCNEGFLLEGARSRVCLANGSWSGATPDCVPVRCATPPQLANGVTEGLDYGFMKEVTFHCHEGYILHG
APKLTCQSDGNWDAEIPLCKPVNCGPPEDLAHGFPNGFSFIHGGHIQYQCFPGYKLHGNSSRRCLSNG
SWSGSSPSCLPCRCSTPVIEYGTVNGTDFDCGKAARIQCFKGFKLLGLSEITCEADGQWSSGFPHCEH
TSCGSLPMIPNAFISETSSWKENVITYSCRSGYVIQGSSDLICTEKGVWSQPYPVCEPLSCGSPPSVA
NAVATGEAHTYESEVKLRCLEGYTMDTDTDTFTCQKDGRWFPERISCSPKKCPLPENITHILVHGDDF
SVNRQVSVSCAEGYTFEGVNISVCQLDGTWEPPFSDESCSPVSCGKPESPEHGFVVGSKYTFESTIIY
QCEPGYELEGNRERVCQENRQWSGGVAICKETRCETPLEFLNGKADIENRTTGPNVVYSCNRGYSLEG
PSEAHCTENGTWSHPVPLCKPNPCPVPFVIPENALLSEKEFYVDQNVSIKCREGFLLQGHGIITCNPD
ETWTQTSAKCEKISCGPPAHVENAIARGVHYQYGDMITYSCYSGYMLEGFLRSVCLENGTWTSPPICR
AVCRFPCQNGGICQRPNACSCPEGWMGRLCEEPICILPCLNGGRCVAPYQCDCPPGWTGSRCHTAVCQ
SPCLNGGKCVRPNRCHCLSSWTGHNCSRKRRTGF

| | | |
|---|---|---|
| NOV8b, 237582568 | SEQ ID NO: 179 | 2127 bp |

| | | |
|---|---|---|
| DNA Sequence | ORF Start: at 1 | ORF Stop: end of sequence |

CGGCCGTGGGCGACCTTTCAGCAGATGTCCCCGTCGCGCAATTTCAGCTTCCGCCTCTTCCCCGAGAC
CGCGCCCGGGGCCCCCGGGAGTATCCCCGCGCCGCCCGCTCCTGGCGACGAAGCGGCGGGGAGCAGAG
TGGAGCGGCTGGGCCAGGCGTTCCGGCGACGCGTGCGGCTGCTGCGGGAGCTCAGCGAGCGCCTGGAG
CTTGTCTTCCTGGTGGATGATTCGTCCAGCGTGGGCGAAGTCAACTTCCGCAGCGAGCTCATGTTCGT
CCGCAAGCTGCTGTCCGACTTCCCCGTGGTGCCCACGGCCACGCGCGTGGCCATCGTGACCTTCTCGT
CCAAGAACTACGTGGTGCCGCGCGTCGATTACATCTCCACCCGCCGCGCGCGCCAGCACAAGTGCGCG
CTGCTCCTCCAAGAGATCCCTGCCATCTCCTACCGAGGTGGCGGCACCTACACCAAGGGCGCCTTCCA
GCAAGCCGCGCAAATTCTTCTTCATGCTAGAGAAAACTCAACAAAAGTTGTATTTCTCATCACTGATG
GATATTCCAATGGGGGAGACCCTAGACCAATTGCAGCGTCACTGCGAGATTCAGGAGTGGAGATCTTC
ACTTTTGGCATATGGCAAGGGAACATTCGAGAGCTGAATGACATGGCTTCCACCCCAAAGGAGGAGCA
CTGTTACCTGCTACACAGTTTTGAAGAATTTGAGGCTTTAGCTCGCCGGGCATTGCATGAAGATCTAC
CTTCTGGGAGTTTTATTCAAGATGATATGGTCCACTGCTCATATCTTTGTGATGAAGGCAAGGACTGC
TGTGACCGAATGGGAAGCTGCAAATGTGGGACACACACAGGCCATTTTGAGTGCATCTGTGAAAAGGG
GTATTACGGGAAAGGTCTGCAGTATGAATGdACAGCTTGCCCATCGGGGACATACAAACCTGAAGCCT
CACCAGGAGGAATCAGCAGTTGCATTCCATGTCCTGATGAAAATCACACCTCTCCACCTGGAAGCACA
TCCCCTGAAGACTGTGTCTGCAGAGAGGGATACAGGGCATCTGGCCAGACCTGTGAACTTGTCCACTG
CCCTGCCCTGAAGCCTCCCGAAAATGGTTACTTTATCCAAAACACTTGCAACAACCACTTCAATGCAG
CCTGTGGGGTCCGATGTCACCCTGGATTTGATCTTGTGGGAAGCAGCATCATCTTATGTCTACCCAAT
GGTTTGTGGTCCGGTTCAGAGAGCTACTGCAGAGTAAGAACATGTCCTCATCTCCGCCAGCCGAAACA
GGTACAGACTAGAAGGCAGTGATAAGCTTACTTGTCAAGGAAACAGCCAGTGGGATGGGCCAGAACCC
CGGTGTGTGGAGCGCCACTGTTCCACCTTTCAGATGCCCAAAGATGTCATCATATCCCCCCACAACTG
TGGCAAGCAGCCAGCCAAATTTGGGACGATCTGCTATGTAAGTTGCCGCCAAGGGTTCATTTTATCTG
GAGTCAAAGAAATGCTGAGATGTACCACTTCTGGAAAATGGAATGTCGGAGTTCAGGCAGCTGTGTGT
AAAGACGTGGAGGCTCCTCAAATCAACTGTCCTAAGGACATAGAGGCTAAGACTCTGGAACAGCAAGA
TTCTGCCAATGTTACCTGGCAGATTCCAACAGCTAAAGACAACTCTGGTGAAAAGGTGTCAGTCCACG
TTCATCCAGCTTTCACCCCACCTTACCTTTTCCCAATTGGAGATGTTGCTATCGTATACACGGCAACT
GACCTATCCGGCAACCAGGCCAGCTGCATTTTCCATATCAAGGTTATTGATGCAGAACCACCTGTCAT
AGACTGGTGCAGATCTCCACCTCCCGTCCAGGTCTCGGAGAAGGTACATGCCGCAAGCTGGGATGAGC
CTCAGTTCTCAGACAACTCAGGGGCTGAATTGGTCATTACCAGAAGTCATACACAAGGAGACCTTTTC
CCTCAAGGGGAGACTATAGTACAGTATACAGCCACTGACCCCTCAGGCAATAATAGGACATGTGATAT
CCATATTGTCATACTCGAG

NOV8b, 237582568

| | | | |
|---|---|---|---|
| Protein Sequence | SEQ ID NO: 180 | 709 aa | MW at 78072.4kD |

RPWATFQQMSPSRNFSFRLFPETAPGAPGSIPAPPAPGDEAAGSRVERLGQAFRRRVRLLRELSERLE
LVFLVDDSSSVGEVNFRSELMFVRKLLSDFPVVPTATRVAIVTFSSKNYVVPRVDYISTRPARQHKCA
LLLQEIPAlSYRGGGTYTKGAFQQAAQILLHARENSTKVVFLITDGYSNGGDPRPIAASLRDSGVEIF
TFGIWQGNIRELNDMASTPKEEHCYLLHSFEEFEALARRALHEDLPSGSFIQDDMVHCSYLCDEGKDC
CDRMGSCKCGTHTGHFECICEKGYYGKGLQYECTACPSGTYKPEASPGGISSCIPCPDENHTSPPGST
SPEDCVCREGYRASGQTCELVHCPALKPPENGYFIQNTCNNHFNAACGVRCHPGFDLVGSSIILCLPN
GLWSGSESYCRVRTCPHLRQPKHGHISCSTREMLYKTTCLVACDEGYRLEGSDKLTCQGNSQWDGPEP
RCVERHCSTFQMPKDVIISPHNCGKQPAKFGTICYVSCRQGFILSGVKEMLRCTTSGKWNVGVQAAVC
KDVEAPQINCPKDIEAKTLEQQDSANVTWQIPTAKDNSGEKVSVHVHPAFTPPYLFPIGDVAIVYTAT
DLSGNQASCIFHIKVIDAEPPVIDWCRSPPPVQVSEKVHAASWDEPQFSDNSGAELVITRSHTQGDLF
PQGETIVQYTATDPSGNNRTCDIHIVILE

| | | |
|---|---|---|
| NOV8c, 236589434 | SEQ ID NO: 181 | 2127 bp |

| | | |
|---|---|---|
| DNA Sequence | ORF Start: at 1 | ORF Stop: end of sequence |

CGGCCGTGGGCGACCTTTCAGCAGATGTCCCCGTCGCGCAATTTCAGCTTCCGCCTCTTCCCCGAGAC
CGCGCCCGGGGCCCCCGGGAGTATCCCCGCGCCGCCCGCTCCTGGCGACGAAGCGGCGGGGAGCAGAG
TGGAGCGGCTGGGCCAGGCGTTCCGGCGACGCGTGCGGCTGCTGCGGGAGCTCAGCGAGCGCCTGGAG
CTTGTCTTCCTGGTGGATGATTCGTCCAGCGTGGGCGAAGTCAACTTCCGCAGCGAGCTCATGTTCGT
CCGCAAGCTGCTGTCCGACTTCCCCGTGGTGCCCACGGCCACGCGCGTGGCCATCGTGACCTTCTCGT
CCAAGAACTACGTGGTGCCGCGCGTCGATTACATCTCCACCCGCCGCGCGCGCCAGCACAAGTGCGCG
CTGCTCCTCCAAGAGATCCCTGCCATCTCCTACCGAGGTGGCGGCACCTACACCAAGGGCGCCTTCCA
GCAAGCCGCGCAAATTCTTCTTCATGCTAGAGAAAACTCAACAAAAGTTGTATCTCTCATCACTGATG
GATATTCCAATGGQGGAGACCCTAGACCAATTGCAGCGTCACTGCGAGATTCAGGAGTGGAGATCTTC
ACTTTTGGCATATGGCAAGGGAACATTCGAGAGCTGAATGACATGGCTTCCACCCCAAAGGAGGAGCA
CTGTTACCTGCTACACAGTTTTGAAGAATTTGAGGCTTTAGCTCGCCGGGCATTGCATGAAGATCTAC
CTTCTGGGAGTTTTATTCAAGATGATATGGTCCACTGCTCATATCTTTGTGATGAAGGCAAGGACTGC
TGTGACCGAATGGGAAGCTGCAAATGTGGGACACACACAGACCGTTTTGAGTGCATCTGTGAAAAGGG
GTATTACGGGAAAGGTCTGCAGTATGAATGCACAGCTTGCCCATCGGGGACATACAAACCTGAAGGCT
CACCAGGAGGAATCAGCAGTTGCATTCCATGTCCTGATGAAAATCACACCTCTCCACCTGGAAGCACA
TCCCCTGAAGACTGTGTCTGCAGAGAGGGATACAGGGCATCTGGCCAGACCTGTGAACTTGTCCACTG
CCCTGCCCTGAAGCCTCCCGAAAATGGTTACTTTATCCAAAACACTTGCAACAACCACTTCAATGCAG
CCTGTGGGGTCCGATGTCACCCTGGATTTGATCTTGTGGGAAGCAGCATCATCTTATGTCTACCCAAT
GGTTTGTGGTCCGGTTCAGAGAGCTACTGCAGAGTAAGAACATGTCCTCATCTCCGCCAGCCGAAACA
TGGCCACATCAGCTGTTCTACAAGGGAAATGTTATATAAGACAACATGTTTGGTTGCCTGTGATGAAG
GGTACAGACTAGAAGGCAGTGATAAGCTTACTTGTCAAGGAAACAGCCAGTGGGATGGGCCAGAACCC
CGGTGTGTGGAGCGCCACTGTTCCACCTTTCAGATGCCCAAAGATATCATCATATCCCCCCACAACTG
TGGCAAGCAGCCAGCCAAATTTGGGACGATCTGCTATGTAAGTTGCCGCCAAGGGTTCATTTTATCTG
GAGTCAAAGAAATGCTGAGATGTACCACTTCTGGAAAATGGAATGTAGGAGTTCAGGCAGCTGTGCGT
AAAGACGTGGAGGCTCCTCAAATCAACTGTCCTAAGGACATAGAGGCTAAGACTCTGGAACAGCAAGA
TTCCGCCAATGTTACCTGGCAGATTCCAACAGCTAAAGACAACTCTGGTGAAAAGGTGTCAGTCCACG
TTCATCCAGCTTTCACCCCACCTTACCTTTTCCCAATTGGAGATGTTGCTATCGTATACACGGCAACT
GACCTATCCGGCAACCAGGCCAGCTGCATTTTCCATATCAAGGTTATTGATGCAGAACCACCTGTCAT
AGACTGGTGCAGATCTCCACCTCCCGTCCAGGTCTCGGAGAAGGTACATGCCGCAAGCTGGGATGAGC
CTCAGTTCTCAGACAACTCAGGGGCTGAATTGGTCATTACCAGAAGTCATACACAAGGAGACCTTTTC
CCTCAAGGGGAGACTATAGTACAGTATACAGCCACTGACCCCTCAGGCAATAACAGGACATGTGATAT
CCATATTGTCATACTCGAG

NOV8c, 236589434

| | | | |
|---|---|---|---|
| Protein Sequence | SEQ ID NO: 182 | 709 aa | MW at 78142.4kD |

RPWATFQQMSPSRNFSFRLFPETAPGAPGSIPAPPAPGDEAAGSRVERLGQAFRRRVRLLRELSERLE
LVFLVDDSSSVGEVNFRSELMFVRKLLSDFPVVPTATRVAIVTFSSKNYVVPRVDYISTRRARQHKCA
LLLQEIPATSYRGGGTYTKGAFQQAAQILLHARENSTKVVSLITDGYSNGGDPRPIAASLRDSGVEIF
TFGIWQGNIRELNDMASTPKEEHCYLLHSFEEFEALARPALHEDLPSGSFIQDDMVHCSYLCDEGKDC
CDRMGSCKCGTHTDRFECICEKGYYGKGLQYECTACPSGTYKPEGSPGGISSCIPCPDENHTSPPGST
SPEDCVCREGYRASGQTCELVHCPALKPPENGYFIQNTCNNHFNAACGVRCHPGFDLVGSSIILCLPN
GLWSGSESYCRVRTCPHLRQPKHGHISCSTREMLYKTTCLVACDEGYRLEGSDKLTCQGNSQWDGPEP
RCVERHCSTFQMPKDIIISPHNCGKQPAKFGTICYVSCRQGFILSGVKEMLRCTTSGKWNVGVQAAVR
KDVEAPQINCPKDIEAKTLEQQDSANVTWQIPTAKDNSGEKVSVHVHPAFTPPYLFPIGDVAIVYTAT
DLSGNQASCIFHIKVIDAEPPVIDWCRSPPPVQVSEKVHAASWDEPQFSDNSGAELVITRSHTQGDLF
PQGETIVQYTATDPSGNNRTCDIHIVILE

| | | |
|---|---|---|
| NOV8d, 236495259 | SEQ ID NO: 183 | 2127 bp |

| | | |
|---|---|---|
| DNA Sequence | ORF Start: at 1 | ORF Stop: end of sequence |

CGGCCGTGGGCGACCTTTCAGCAGATGTCCCCGTCGCGCAATTTCAGCTTCCGCCTCTTCCCCGAGAC
CGCGCCCGGGGCCCCCGGGAGTATCCCCGCGCCGCCCGCTCCTGGCGACGAAGCGGCGGGGAGCAGAG
TGGAGCGGCTGGGCCAGGCGTTCCGGCGACGCGTGCGGCTGCTGCGGGAGCTCAGCGAGCGCCTGGAG
CTTGTCTTCCTGGTGGATGATTCGTCCAGCGTGGGCGAAGTCAACTTCCGCAGCGAGCTCATGTTCGT
CCGCAAGCTGCTGTCCGACTTCCCCGTGGTGCCCACGGCCACGCGCGTGGCCATCGTGACCTTCTCGT
CCAAGAACTACGTGGTGCCGCGCGTCGATTACATCTCCACCCGCCGCGCGCGCCAGCACAAGTGCGCG
CTGCTCCTCCAAGAGATCCCTGCCATCTCCTACCGAGGTGGCGGCACCTACATCAAGGGCGCCTTCCA
GCAAGCCGCGCAAATTCTTCTTCATGCTAGAGAAAACTCAACAAAAGTTGTATTTCTCATCACTGATG
GATATTCCAATGGGGGAGACCCTAGACCAATTGCAGCGTCACTGCGAGATTCAGGAGTGGAGATCTTC
ACTTTTGGCATATGGCAAGGGAACATTCGAGAGCTGAATGACATGGCTTCCACCCCAAAGGAGGAGCA
CTGTTACCTGCTACACAGTTTTGAAGAATTTGAGGCTTTAGCTCGCCGGGCATTGCATGAAGATCTAC
CTTCTGGGAGTTTTATTCAAGATGATATGGTCCACTGCTCATATCTTTGTGATGAAGGCAAGGACTGC
TGTGACCGAATGGGAAGCTGCAAATGTGGGACACACACAGGCCATTTTGAGTGCATCTGTGAAAAGGG
GTATTACGGGAAAGGTCTGCAGTATGAATGCACAGCTTGCCCATCGGGGACATACAAACCTGAAGGCT
CACCAGGAGGAATCAGCAGTTGCATTCCATGTCCTGATGAAAATCACACCTCTCCACCTGGAAGCACA
TCCCCTGAAGACTGTGTCTGCAGAGAGGOATACAGGGCATCTGGCCAGACCTGTGAACTTGTCCACTG
CCCTGCCCTGAAGCCTCCCGAAAATGGTTACTTTATCCAAAACACTTGCAACAACCACTTCAATGCAG
CCTGTGGGGTCCGATGTCACCCTGGATTTGATCTTGTGGGAAGCAGCATCATCTTATGTCTACCCAAT
GGTTTGTGGTCCGGTTCAGAGAGCTACTGCAGAGTAAGAACATGTCCTCATCTCCGCCAGCCGAAACA
TGGCCACATCAGCTGTTCTACAAGGGAAATGTTATATAAGACAACATGTTTGGTTGCCTGTGATGAAG
GGTACAGACTAGAAGGCAGTGATAAGCTTACTTGTCAAGGAAACAGCCAGTGGGATGGGCCAGAACCC
CGGTGTGTGGAGCGCCACTGTTCCACCTTTCAGATGCCCAAAGATGTCATCATATCCCCCCACAACTG
TGGCAAGCAGCCAGCCAAATTTGGGACGATCTGCTATGTAAGTTGCCGCCAAGGGTTCATTTTATCTG
GAGTCAAAGAAATGCTGAGATGCACCACTTCTGGAAAATGGAATGTCGGAGTTCAGGCAGCTGTGTGT
AAAGACGTGGAGGCTCCTCAAATCAACTGTCCTAAGGACATAGAGGCTAAGACTCTGGAACAGCAAGA
TTCTGCCAATGTTACCTGGCAGATTCCAACAGCTAAAGACAACTCTGGTGAAAAGGTGTCAGTCCACG
TTCATCCAGCTTTCACCCCACCTTACCTTTTCCCAATTGGAGATGTTGCTATCGTATACACGGCAACT
GACCTATCCGGCAACCAGGCCAGCTGCATTTTCCATATCAAGGTTATTGATGCAGAACCACCTGTCAT
AGACTGGTGCAGATCTCCACCTCCCGTCCAGGTCTCGGAGAAGGTACATGCCGCAAGCTGGGATGAGC
CTCAGTTCTCAGACAACTCAGGGGCCGAATTGGTCATTACCAGAAGTCATACACAAGGAGACCTTTTC
CCTCAAGGGGAGACTATAGTACAGTATACAGCCACTGACCCCTCAGGCAATAACAGGACATGTGATAT
CCATATTGTCATACTCGAG

NOV8d, 236495259

| | | | |
|---|---|---|---|
| Protein sequence | SEQ ID NO: 184 | 709 aa | MW at 78070.4kD |

RPWATFQQMSPSRNFSFRLFPETAPGAPGSIPAPPAPGDEAAGSRVERLGQAFRRRVRLLRELSERLE
LVFLVDDSSSVGEVNFRSELMFVRKLLSDFPVVPTATRVAIVTFSSKNYVVPRVDYISTRRARQHKCA
LLLQEIPAlSYRGGGTYIKGAFQQAAQILLHARENSTKVVFLITDGYSNGGDPRPIAASLRDSGVEIF
TFGIWQGNIRELNDMASTPKEEHCYLLHSFEEFEALARRALHEDLPSGSFIQDDMVHCSYLCDEGKDC
CDPMGSCKCGTHTGHFECICEKGYYGKGLQYECTACPSGTYKPEGSPGGISSCIPCPDENHTSPPGST
SPEDCVCREGYRASGQTCELVHCPALKPPENGYFIQNTCNNHFNAACGVRCHPGFDLVGSSIILCLPN
GLWSGSESYCRVRTCPHLRQPKHGHISCSTREMLYKTTCLVACDEGYRLEGSDKLTCQGNSQWDGPEP
RCVERNCSTFQMPKDVIISPHNCGKQPAKFGTICYVSCRQGFILSGVKEMLRCTTSGKWNVGVQAAVC
KDVEAPQINCPKDIEAKTLEQQDSANVTWQIPTAKDNSGEKVSVHVHPAFTPPYLFPIGDVAIVYTAT
DLSGNQASCIFHIKVIDAEPPVIDWCRSPPPVQVSEKVHAASWDEPQFSDNSGAELVITRSHTQGDLF
PQGETIVQYTATDPSGNNRTCDIHIVILE

| | | |
|---|---|---|
| NOV8e, 236495256 | SEQ ID NO: 185 | 2127 bp |

| | | |
|---|---|---|
| DNA Sequence | ORF Start: at 1 | ORF Stop: end of sequence |

CGGCCGTGGGCGACCTTTCAGCAGATGTCCCCGTCGCGCAATTTCAGCTTCCGCCTCTTCCCCGAGAC
CGCGCCCGGGGCCCCCGGGAGTATCCCCGCGCCGCCCGCTCCTGGCGACGAAACGGCGGGGAGCAGAG
TGGAGCGGCTGGGCCAGGCGTTCCGGCGACGCGTGCGGCTGCTGCGGGAGCTCAGCGAGCGCCTGGAG
CTTGTCTTCCTGGTGGATGATTCGTCCAGCGTGGGCGAAGTCAACTTCCGCAGCGAGCTCATGTTCGT
CCGCAAGCTGCTGTCCGACTTCCCCGTGGTGCCCACGGCCACGCGCGTGGCCATCGTGACCTTCTCGT
CCAAGAACTACGTGGTGCCGCGCGCCGATTACATCTCCACCCGCCGCGCGCGCCAGCACAAGTGCGCG
CTGCTCCTCCAAGAGATCCCTGCCATCTCCTACCGGGGTGGCGGCACCTACACCAAGGGCGCCTTCCA
GCAAGCCGCGCAAATTCTTCTTCATGCTAGAGAAAACTCAGCAAAAGTTGTATTTCTCATCACTGATG
GATATTCCAATGGGGGAGACCCTAGACCAATTGCAGCGTCACTGCGAGATTCAGGAGTGGAGATCTTC
ACTTTTGGCATATGGCAAGGGAACATTCGAGAGCTGAATGACATGGCTTCCACCCCAAAGGAGGAGCA
CTGTTACCTGCTACACAGTTTTGAAGAATTTGAGGCTTTAGCTCGCCGGGCATTGCATGAAGATCTAC
CTTCTGGGAGTTTTATTCAAGATGATATGGTCCACTGCTCATATCTTTGTGATGAAGGCAAGGACTGC
TGTGACCGAATGGGAAGCTGCAAATGTGGGACACACACAGGCCATTTTGAGTGCATCTGTGAAAAGGG
GTATTACGGGAAGGGTCTGCAGTATGAATGCACAGCTTGCCCATCGGGGACATACAAACCTGAAGGCT
CACCAGGAGGAATCAGCAGTTGCATTCCATGTCCTGATGAAAATCACACCTCTCCACCTGGAAGCACA
TCCCCTGAAGACTGTGTCTGCAGAGAGGGATACAGGGCATCTGGCCAGACCTGTGAACTTGTCCACTG
CCCTGCCCTGAAGCCTCCCGAAAATGGTTACTTTATCCAAAACACTTGCAACAACCACTTCAATGCAG
CCTGTGGGGTCCGATGTCACCCTGGATTTGATCTTGTGGGAAGCAGCATCATCTTATGTCTACCCAAT
GGTTTGTGGTCCGGTTCAGAGAGCTACTGCAGAGTAAGAACATGTCCTCATCTCCGCCAGCCGAAACA
TGGCCACATCAGCTGTTCTACAAGGGAAATGTTATATAAGACAACATGTTTGGTTGCCTGTGATGAAG
GGTACAGACTAGAAGGCAGTGATAAGCTTACTTGTCAAGGAAACAGCCAGTGGGATGGGCCAGAACCC
CGGTGTGTGGAGCGCCACTGTTCCACCTTTCAGATGCCCAAAGATATCATCATATCCCCCCACAACTG
TGGCAAGCAGCCAGCCAAATTTGGGACGATCTGCTATGTAAGTCGCCGCCAAGGGTTCATTTTATCTG
GAGTCAAAGAAATGCTGAGATGTACCACTTCTGGAAAATGGAATGTCGGAGTTCAGGCAGCTGTGTGT
AAAGACGTGGAGGCTCCTCAAATCAACTGTCCTAAGGACATAGAGGCTAAGACTCTGGAACAGCAAGA
TTCTGCCAATGTTACCTGGCAGATTCCAACAGCTAAAGACAACTCTGGTGAAAAGGTGTCAGTCCACG
TTCATCCAGCTTTCACCCCACCTTACCTTTTCCCAATTGGAGATGTTGCTATCGTATACACGGCAACT
GACCTATCCGGCAACCAGGCCAGCTGCATTTTCCATATCAAGGTTATTGATGCAGAACCACCTGTCAT
AGACTGGTGCAGATCTCCACCTCCCGTCCAGGTCTCGGAGAAGGTACATGCCGCAAGCTGGGATGAGC
CTCAGTTCTCAGACAACTCAGGGGCTGAATTGGTCATTACCAGAAGTCATACACAAGGAGAACTTTTC
CCTCAAGGGGAGACTATAGTACAGTATACAGCCACTGACCCCTCAGGCAATAACAGGACATGTGATAT
CCATATTGTCATACTCGAG

NOV8e, 236495256

| | | | |
|---|---|---|---|
| Protein Sequence | SEQ ID NO: 186 | 709 aa | MW at 78081.3kD |

RPWATFQQMSPSRNFSFRLFPETAPGAPGSIPAPPAPGDEAAGSRVERLGQAFRRRVRLLRELSERLE
LVFLVDDSSSVGEVNFRSELMFVRKLLSDFPVVPTATRVAIVTFSSKNYVVPRADYISTRRARQHKCA
LLLQEIPAISYRGGGTYTKGAFQQAAQILLHARENSAKVVFLITDGYSNGGDPRPIAASLRDSGVEIF
TFGIWQGNIRELNDMASTPKEEHCYLLHSFEEFEALARRALHEDLPSGSFIQDDMVHCSYLCDEGKDC
CDRMGSCKCGTHTGHFECICEKGYYGKGLQYECTACPSGTYKPEGSPGGISSCIPCPDENHTSPPGST
SPEDCVCREGYRASGQTCELVHCPALKPPENGYFIQNTCNNHFNAACGVRCHPGFDLVGSSIILCLPN
GLWSGSESYCRVRTCPHLRQPKHGHISCSTREMWYKTTCLVACDEGYPLEGSDKLTCQGNSQWDGPEP
RCVERHCSTFQMPKDIIISPHNCGKQPAKFGTICYVSRRQGFILSGVKEMLRCTTSGKWNVGVQAAVC
KDVEAPQINCPKDIEAKTLEQQDSANVTWQIPTAKDNSGEKVSVHVHPAFTPPYLFPIGDVAIVYTAT
DLSGNQASCIFHIKVIDAEPPVIDWCRSPPPVQVSEKVHAASWDEPQFSDNSGAELVITRSHTQGELF
PQGETIVQYTATDPSGNNRTCDIHIVILE

| | | |
|---|---|---|
| NOV8f, CG50646-01 | SEQ ID NO: 187 | 1952 bp |

| | | |
|---|---|---|
| DNA Sequence | ORF Start: ATG at 470 | ORF Stop: TGA at 1688 |

TTCAGGAACACCGTCACTTACACTTGCAAAGAAGGCTATACTCTTGCTGGTCTTGACACCATTGAATG
CCTGGCCGACGGCAAGTGGAGTAGAAGTGACCAGCAGTGCCTGGCTGTCTCCTGTGATGAGCCACCCA
TTGTGGACCACGCCTCTCCAGAGACTGCCCATCGGCTCTTTGGAGACATTGCATTCTACTACTGCTCT
GATGGTTACAGCCTAGCAGACAATTCCCAGCTTCTCTGCAATGCCCAGGGCAAGTGGGTACCCCCAGA
AGGTCAAGACATGCCCCGTTGTATAGCTCATTTCTGTGAAAAACCTCCATCGGTTTCCTATAGCATCT
TGGAATCTGTGAGCAAAGCAAAATTTGCAGCTGGCTCAGTTGTGAGCTTTAAATGCATGGAAGGCTTT
GTACTGAACACCTCAGCAAAGATTGAATGTATGAGAGGTGGGCAGTGGAACCCTTCCCCCCATGTCCA
TCCAGTGCATCCCTGTGCGGTGTGGAGAGCCACCAAGCATCATGAATGGCTATGCAAGTGGATCAAAC
TACAGTTTTGGAGCCATGGTGGCTTACAGCTGCAACAAGGGGTTCTACATCAAAGGGGAAAAGAAGAG
CACCTGCGAAGCCACAGGGCAGTGGAGTAGTCCTATACCGACGTGCCACCCGGTATCTTGTGGTGAAC
CACCTAAGGTTGAGAATGGCTTTCTGGAGCATACACCTGGCAGGATCTTTGAGAGTGAAGTGAGGTAT
CAGTGTAACCCGGGCTATAAGTCAGTCGGAAGTCCTGTATTTGTCTGCCAAGCCAATCGCCACTGGCA
CAGTGAATCCCCTCTGATGTGTGTTCCTCTCGACTGTGGAAAACCTCCCCCGATCCAGAATGGCTTCA
TGAAAGGAGAAAACTTTGAAGTAGGGTCCAAGGTTCAGTTTTTCTGTAATGAGGGTTATGAGCTTGTT
GGGGACAGTTCTTGGACATGTCAGAAATCTGGCAAATGGAATAAGAAGTCAAATCCAAAGTGCATGCC
TGCCAAGTGCCCAGAGCCGCCCCTCTTGGAAAACCAGCTAGTATTAAAGGAGTTGACCACCGAGGTAG
GAGTTGTGACATTTTCCTGTAAAGAAGGGCATGTCCTGCAAGGCCCCTCTGTCCTGAAATGCTTGCCA
TCCCAGCAATGGAATGACTCTTTCCCTGTTTGTAAGATTGTTCTTTGTACCCCACCTCCCCTAATTTC
CTTTGGTGTCCCCATTCCTTCTTCTGCTCTTCATTTTGGAAGTACTGTCAAGTATTCTTGTGTAGGTG
GGTTTTTCCTAAGAGGAAATTCTACCACCCTCTGCCAACCTGATGGCACCTGGAGCTCTCCACTGCCA
GAATGTGTTCCAGTAGAATGTCCCCAACCTGAGGAAATCCCCAATGGAATCATTGATGTGCAAGGCCT
TGCCTATCTCAGCACAGCTCTCTATACCTGCAAGCCAGGCTTTGAATTGGTGGGAAATACTACCACCC
TTTGTGGAGAAAATGGTCACTGGCTTGGAGGAAAACCAACATGTAAAGCCATTGAGTGCCTGAAACCC
AAGGAGATTTTGAATGGCAAATTCTCTTACACGGACCTACACTATGGACAGACCGTTACCTACTCTTG
CAACCGAGGCTTTCGGCTCGAAGGGTCCCAGTGCCTTGACCTGTTTAGAGACAGGTGATTGGGATGTA
GATGCCCCATCTTGCAATGCCATCCACTGTGATTCCCCACAACCCATTGAAAATGGTTTTGTAGAAGC
TGCAGATTACAGCTATGGTGCCATAATCATCTACAGTTGCTTCCCTGGGTTTCAGGTGGCTGGTCATC
CTCCCTCCTCATATAGATTTTGGAGACTGTACTAAACTCAAAGATGAC

NOV8f, CG50646-01

| | | | |
|---|---|---|---|
| Protein Sequence | SEQ ID NO: 188 | 406 aa | MW at 44385.5kD |

MSIQCIPVRCGEPPSIMNGYASGSNYSFGAMVAYSCNKGFYIKGEKKSTCEATGQWSSPIPTCHPVSC
GEPPKVENGFLEHTTGRIFESEVRYQCNPGYKSVGSPVFVCQANRHWHSESPLMCVPLDCGKPPPIQN
GFMKGENFEVGSKVQFFCNEGYELVGDSSWTCQKSGKWNKKSNPKCMPAKCPEPPLLENQLVLKELTT
EVGVVTFSCKEGHVLQGPSVLKCLPSQQWNDSFPVCKIVLCTPPPLISFGVPIPSSALHFGSTVKYSC
VGGFFLRGNSTTLCQPDGTWSSPLPECVPVECPQPEEIPNGIIDVQLAYLSTALYTCKPGFELVGNT
TTLCGENGHWLGGKPTCKAIECLKPKEILNGKFSYTDLHYGQTVTYSCNRGFRLEGSQCLDLFRDR

| | | |
|---|---|---|
| NOV8g, CG50646-02 | SEQ ID NO: 189 | 1709 bp |

| | | |
|---|---|---|
| DNA Sequence | ORF Start: ATG at 413 | ORF Stop: TGA at 1445 |

CAGAGTCTTGCTCTGTCTCCCAGGCTGGAGTGCAGTGGCACAATCTCAGCTCACTGCAACCTCTGCCT
CCTGGGTTCAAGTGATTCTCCTGCCTCAGCTTCCCAAATGGCTGAGATTACAGGCACATACCACCATG
CCTAGCTAATTTTTGTACAGGTTTCACCATGTTGGCCAGGCTGGTCTCGAACTCCTAACCTCAAGTGT
TCCTCCTGCCTCCGCCTCCCAAAGTGCTGGGATTGTAGGCATGAATCGTCATGCCGAGCCTAAGTTGA
CTTTCTACTATCATTTTCACTTATTTAAAAAAATAGAATGGATCTATTGGAAAAACCATAAATCATTA
TTTGCTTACTTCCTAATTGATTCATTTTAACATAGACCTTTTAGTTTTTTTCACTATCCAAGGATTTA
GTTAATGCTATCATCTGTTATACAAATCGCACTCACTTGCTTTCTTCCTGTTGCACAGCATACAACTG
GCAGGATCTTTGAGAGTGAAGTGAGGTATCAGTGTAACCCGGGCTATAAGTCAGTCGGAAGTCCTGTA
TTTGTCTGCCAAGCCAATCGCCACTGGCACAGTGAATCCCCTCTGATGTGTGTTCCTCTCGACTGTGG
AAAACCTCCCCCGATCCAGAATGGCTTCATGAAAGGAGAAAACTTTGAAGTAGGGTCCAAGGTTCAGT
TTTTCTGTAATGAGGGTTATGAGCTTGTTGGGGACAGTTCTTGGACATGTCAGAAATCTGGCAAATGG
AATAAGAAGTCAAATCCAAAGTGCATGCCTGCCAAGTGCCCAGAGCCGCCCCTCTTGGAAAACCAGCT
AGTATTAAAGGAGTTGACCACCGAGGTAGGAGTTGTGACATTTTCCTGTAAAGAAGGGCATGTCCTGC
AAGGCCCCTCTGTCCTGAAATGCTTGCCATCCCAGCAATGGAATGACTCTTTCCCTGTTTGTAAGATT
GTTCTTTGTACCCCACCTCCCCTAATTTCCTTTGGTGTCCCCATTCCTTCTTCTGCTCTTCATTTTGG
AAGTACTGTCAAGTATTCTTGTGTAGGTGGGTTTTTCCTAAGAGGAAATTCTACCACCCTCTGCCAAC
CTGATGGCACCTGGAGCTCTCCACTGCCAGAATGTGTTCCAGTAGAATGTCCCCAACCTGAGGAAATC
CCCAATGGAATCATTGATGTGCAAGGCCTTGCCTATCTCAGCACAGCTCTCTATACCTGCAAGCCAGG
CTTTGAATTGGTGGGAAATACTACCACCCTTTGTGGAGAAAATGGTCACTGGCTTGGAGGAAAACCAA
CATGTAAAGCCATTGAGTGCCTGAAACCCAAGGAGATTTTGAATGGCAAATTCTCTTACACGGACCTA
CACTATGGACAGACCGTTACCTACTCTTGCAACCGAGGCTTTCGGCTCGAAGGGTCCCAGTGCCTTGA
CCTGTTTAGAGACAGGTGATTGGGATGTAGATGCCCCATCTTGCAATGCCATCCACTGTGATTCCCCA
CAACCCATTGAAAATGGTTTTGTAGAAGGTGCAGATTACAGCTATGGTGCCATAATCATCTACAGTTG
CTTCCCTGGGTTTCAGGTGGCTGGTCATGCCATGCAGACCTGTGAAGAGTCAGGATGGTCACTCGTGC
CCCCAACATGTATGCCAATAGACTGTGGCCTCCCTCCTCATATAGATTTTGGAGACTGTACTAAACTC
AAAGATGAC

NOV8g, CG50646-02

| | | | |
|---|---|---|---|
| Protein Sequence | SEQ ID NO: 190 | 344 aa | MW at 37763.2kD |

MLSSVIQIALTCFLPVAQHTTGRIFESEVRYQCNPGYKSVGSPVFVCQANRHWHSESPLMCVPLDCGK
PPPIQNGFMKGENFEVGSKVQFFCNEGYELVGDSSWTCQKSGKWNKKSNPKCMPAKCPEPPLLENQLV
LKELTTEVGVVTFSCKEGHVLQGPSVLKCLPSQQWNDSFPVCKIVLCTPPPLISFGVPIPSSALHFGS
TVKYSCVGGFFLRGNSTTLCQPDGTWSSPLPECVPVECPQPEEIPNGIIDVQGLAYLSTALYTCKPGF
ELVGNTTTLCGENGHWLGGKPTCKAIECLKPKEILNGKFSYTDLHYGQTVTYSCNRGFRLEGSQCLDL
FRDR

| | | |
|---|---|---|
| NOV8h, CG50646-03 | SEQ ID NO: 191 | 6153 bp |

| | | |
|---|---|---|
| DNA Sequence | ORF Start: ATG at 1 | ORF Stop: TGA at 6151 |

ATGGCGGGCGCCCCTCCCCCAGCCTCGTTGCCGCCTTGCAGTTTGATCTCAGACTGCTGTGCTAGCAA
TCAGCGAGATTCCGTGGGCGTAGGACCCTCTGAGCCAGGTGTGGGATATAGTCTCGTGGTGCGCCGTT
TCTTAAGCCGGTCTGAAAAGCGCAATATTCGGGTGGGAGTGACCCGATTTTCCAGCTATACTCTTGCT
CTCCTGTGATGAGCCACCCATTGTGGACCACGCCTCTCCAGAGACTGCCCATCGGCTCTTTGGAGACA
TTGCATTCTACTACTGCTCTGATGGTTACAGCCTAGCAGACAATTCCCAGCTTCTCTGCAATGCCCAG
GGCAAGTGGGTACCCCCAGAAGGTCAAGACATGCCCCGTTGTATAGCTCATTTCTGTGAAAAACCTCC
ATCGGTTTCCTATAGCATCTTGGAATCTGTGAGCAAAGCAAAATTTGCAGCTGGCTCAGTTGTGAGCT
AACCCTTCCCCCATGTCCATCCAGTGCATCCCTGTGCGGTGTGGAGAGCCACCAAGCATCATGAATGG
TCAAAGGGGAAAAGAAGAGCACCTGCGAAGCCACAGGGCAGTGGAGTAGTCCTATACCGACGTGCCAC
CCGGTATCTTGTGGTGAACCACCTAAGGTTGAGAATGGCTTTCTGGAGCATACAACTGGCAGGATCTT
TGAGAGTGAAGTGAGGTATCAGTGTAACCCGGGCTATAAGTCAGTCGGAAGTCCTGTATTTGTCTGCC
AAGCCAATCGCCACTGGCACAGTGAATCCCCTCTGATGTGTGTTCCTCTCGACTGTGGAAAACCTCCC
CCGATCCAGAATGGCTTCATGAAAGGAGAAAACTTTGAAGTAGGGTCCAAGGGTCAGTTTTTCTGTAA
TGAAGGGTTATNGAGCTTTGTTGGGGACAGTTCTTGGACATGTCAGAAATCTGGCAAATGGAATAAGA
AGTCAAATCCAAAGTGCATGCCTGCCAAGTGCCCAGAGCCGCCCCTCTTGGAAAACCAGCTAGTATTA
AAGGAGTTGACCACCGAGGTAGGAGTTGTGACATTTTCCTGTAAAGAAAGGCATGTCCTGCAAGGCCC
CTCTGTCCTGAAATGCTTGCCATCCCAGCAATGGAATGACTCTTTCCCTGTTTGTAAGATTGTTCTTT
GTACCCCACCTCCCCTAATTTCCTTTGGTGTCCCCATTCCTTCTTCTGCTCTTCATTTTGGAAGTACT
GTCAAGTATTCTTGTGTAGGTGGGTTTTTCCTAAGAGGAAATTCTACCACCCTCTGCCAACCTGATGG
CACCTGGAGCTCTCCACTGCCAGAATGTGTTCCAGTAGAATGTCCCCAACCTGAGGAAATCCCCAATG
GAATCATTGATGTGCAAGGCCTTGCCTATCTCAGCACAGCTCTCTATACCTGCAAGCCAGGCTTTGAA
TTGGTGGGAAATACTACCACCCTTTGTGGAGAAAATGGTCACTGGCTTGGAGGAAAACCAACATGTAA
AGCCATTGAGTGCCTGAAACCCAAGGAGATTTTGAATGGCAAATTCTCTTACACGGACCTACACTATG
GACAGACCGTTACCTACTCTTGCAACCGAGGCTTTCGGCTCGAAGGTCCCAGTGCCTTGACCTGTTTA
GAGACAGGTGATTGGGATGTAGATGCCCCATCTTGCAATGCCATCCACTGTGATTCCCCACAACCCAT
TGAAAATGGTTTTGTAGAAGGTGCAGATTACAGCTATGGTGCCATAATCATCTACAGTTGCTTCCCTG
GGTTTCAGGTGGCTGGTCATGCCATGCAGACCTGTGAAGAGTCAGGATGGTCAAGTTCCATCCCAACA
TGTATGCCAATAGACTGTGGCCTCCCTCCTCATATAGATTTTGGAGACTGTACTAAACTCAAAGATGA
CCAGGGATATTTTGAGCAAGAAGACGACATGATGGAAGTTCCATATGTGACTCCTCACCCTCCTTATC
ATTTGGGAGCAGTGGCTAAAACCTGGGAAAATACAAAGGAGTCTCCTGCTACACATTCATCAAACTTT
CTGTATGGTACCATGGTTTCATACACCTGTAATCCAGGATATGAACTTCTGGGGAACCCTGTGCTGAT
CTGCCAGGAAGATGGAACTTGGAATGGCAGTGCACCATCCTGCATTTCAATTGAATGTGACTTGCCTA
CTGCTCCTGAAAATGGCTTTTTGCGTTTTACAGAGACTAGCATGGGAAGTGCTGTGCAGTATAGCTGT
AAACCTGGACACATTCTAGCAGGCTCTGACTTAAGGCTTTGTCTAGAGAATAGAAAGTGGAGTGGTGC
CTCCCCACGCTGTGAAGCCATTTCATGCAAAAAGCCAAATCCAGTCATGAATGGATCCATCAAAGGAA
GCAACTACACATACCTGAGCACGTTGTACTATGAGTGTGACCCCGGATATGTGCTGAATGGCACTGAG
AGGAGAACATGCCAGGATGACAAAAACTGGGATGAGGATGAGCCCATTTGCATTCCTGTGGACTGCAG
TTCACCCCCAGTCTCAGCCAATGGCCAGGTGAGAGGAGACGAGTACACATTCCAAAAAGAGATTGAAT
ACACTTGCAATGAAGGGTTCTTGCTTGAGGGAGCCAGGAGTCGGGTTTGTCTTGCCAATGGAAGTTGG
AGTGGAGCCACTCCCGACTGTGTGCCTGTCAGATGTGCCACCCCGCCACAACTGGCCAATGGGGTGAC
GGAAGGCCTGGACTATGGCTTCATGAAGGAAGTAACATTCCACTGTCACGAGGGCTACATCTTGCACG
GTGCTCCAAAACTCACCTGTCAGTCAGATGGCAACTGGGATGCAGAGATTCCTCTCTGTAAACCAGTC
AACTGTGGACCTCCTGAAGATCTTGCCCATGGTTTCCCTAATGGTTTTTCCTTTATTCATGGGGGCCA
TATACAGTATCAGTGCTTTCCTGGTTATAAGCTCCATGGAAATTCATCAAGAAGGTGCCTCTCCAATG
GCTCCTGGAGTGGCAGCTCACCTTCCTGCCTGCCTTGCAGATGTTCCACACCAGTAATTGAATATGGA
ACTGTCAATGGGACAGATTTTGACTGTGGAAAGGCAGCCCGGATTCAGTGCTTCAAAGGCTTCAAGCT
CCTAGGACTTTCTGAAATCACCTGTGAAGCCGATGGCCAGTGGAGCTCTGGGTTCCCCCACTGTGAAC
ACACTTCTTGTGGTTCTCTTCCAATGATACCAAATGCGTTCATCAGTGAGACCAGCTCTTGGAAGGAA
AATGTGATAACTTACAGCTGCAGGTCTGGATATGTCATACAAGGCAGTTCAGATCTGATTTGTACAGA
GAAAGGGGTATGGAGCCAGCCTTATCCAGTCTGTGAGCCCTTGTCCTGTGGGTCCCCACCGTCTGTCG
CCAATGCAGTGGCAACTGGAGAGGCACACACCTATGAAAGTGAAGTGAAACTCAGATGTCTGGAAGGT
TATACGATGGATACAGATACAGATACATTCACCTGTCAGAAAGATGGTCGCTGGTTCCCTGAGAGAAT
CTCCTGCAGTCCTAAAAAATGTCCTCTCCCGGAAAACATAACACATATACTTGTACATGGGGACGATT
TCAGTGTGAATAGGCAAGTTTCTGTGTCATGTGCAGAAGGGTATACCTTTGAGGGAGTTAACATATCA
GTATGTCAGCTTGATGGAACCTGGGAGCCACCATTCTCCGATGAATCTTGCAGTCCAGTTTCTTGTGG
GAAACCTGAAAGTCCAGAACATGGATTTGTGGTTGGCAGTAAATACACCTTTGAAAGCACAATTATTT
ATCAGTGTGAGCCTGGCTATGAACTAGAGAATTTGGCTGTGAATCCATCTGGTCCTGGACTTTTCTTG
ATCAGCAGCAGAGGGTGCAGAACAGCGGATATTGGTGAACCGCAAATGCTGCTGCCTGATCATTCCTC
TGGAAGTTTTGTCTCAGAGGAATACCCGGCCATGTGAGGTGTCAGTCCGCCCCTACTGGGGGGGGAAC
AGGGAACGTGTCTGCCAGGAGAACAGACAGTGGAGTGGAGGGGTGGCAATATGCAAAGAGACCAGGTG
TGAAACTCCACTTGAATTTCTCAATGGGAAAGCTGACATTGAAAACAGGACGACTGGACCCAACGTGG
TATATTCCTGCAACAGAGGCTACAGTCTTGAAGGGCCATCTGAGGCACACTGCACAGAAAATGGAACC
TGGAGCCACCCAGTCCCTCTCTGCAAACCAAATCCATGCCCTGTTCCTTTTGTGATTCCCGAGAATGC
TCTGCTGTCTGAAAAGGAGTTTTATGTTGATCAGAATGTGTCCATCAAATGTAGGGAAGGTTTTCTGC
TGCAGGGCCACGGCATCATTACCTGCAACCCCGACGAGACGTGGACACAGACAAGCGCCAAATGTGAA
AGAAGATATACACAACAGCCCAAGTCCCTGAATTTTCAGCTAGCAGCTTATTGCAGTATTAGAATGTT
TATTTTGCGGGGAGGGGTTCAAGATGGCCAACTAGAAACAGCTGTGGCCGGAGCCTCCCACCGAGAAG
AACAAAAACAAAAGCGAGAAAAAGCAAGGTGGTACAACGGCCCACCTGGGAGCCACATGGGGCAAGCA
GAGCTCCCACCCCCAGCCAAAGGAGGTGGACCTCCCTGCGGGAATTTCAGCAACTCCAGCCAGGGGTT
TATGAACAGACCTCTGATCTCCCTGAGATGGAGCCCCTGGGGCTCCATGTGGCCATGGTCTCCACAGA
AACAGCACAGTTTACCTGCTCTGCCAAGGGGCAGCTAGAGCGCTTTGTTAAGCGAGTCCCTGATCCCA
GAGCTCCCACCCCCAGCCAAAGGAGGTGGACCTCCCTGCGGGAATTTCAGCAACTCCAGCCAGGGGTT
TATGAACAGACCTCTGATCTCCCTGAGATGGAGCCCCTGGGGCTCCATGTGGCCATGGTCTCCACAGA
TCAGCAGGCTTAGTCCTTCCCCTGCTGGCTCTGAGGAATCCAGGCAGGCTGGACTAGTGGGATTCCCC
ACAGCACAGTTTACCTGCTCTGCCAAGGGGCAGCTAGAGCGCTTTGTTAAGCGAGTCCCTGATCCCAT
GCCTCCTGATTGGGATGAGACCCCCCCACAACAGGGGTCACGGATGAGACCCCCCCACAACAGGGGTC
ACCAGACACCTTATACAAGGGTGTTCCTGCTAGCATCAGGTCAGTGCCCCTCTGGGACAGAGCTCCCA
GAGGAAAGAGCAGGCAGCCATCTTTGCTGTTCTGCAGCGTCCGCTGGAAAAGCACAGAATTGGGCAGA
GGCTAGGATTGATGAATTGAAAGAAGTAGGCTTCAGAAAGTGGGTAATAATGAAGTTCGCTGAGCTAA
AGGAACATGTTCTAAACCAATGCAIAGACGCCAAGAACCAGGATAAAACATTACAGGATCCGTTAACC
AGAATAACCAGTTTAGAAAGGAATGTAAATGACCTGATGGAGCTGAAAAACACAACACGAGAACTTCA
CAATGCAACAACAAAACAAGGCCAACATTCFAGTTCAGGAAATCCAGAGAACCCCAGTAAGATACTCC
ATGAGAAGATCAACCCCAAGACACATAATCCTCAGGTTCTCCAAGAAATCTCATGTGGTCCACCAGCT
CACGTAGAAAATGCAATTGCTCGAGGCGTACATTATCAATATGGAGACATGATCACCTACTCATGTTA
CAGTGGATACATGTTGGAGGGTTTCCTGAGGAGTGTTTGTTTAGAAAATGGAACATGGACATCACCTC
CTATTTGCAGAGCTGTCTGTCGATTTCCATGTCAGAATGGGGGCATCTGCCAACGCCCAAATGCTTGT
TCCTGTCCAGAGGGCTGGATGGGGCGCCTCTGTGAAGAACCAATCTGCATTCTTCCCTGTCTGAACGG
AGGTCGCTGTGTGGCCCCTTACCAGTGTGACTGCCCGCCTGGCTGGACGGGGTCTCGCTGTCATACAG
GTAGGCCTCTTTCATGGTTTGTTTTCTTGGTGGCTCAGGCCCATGAAACTCCAGAGGACATTGAAGAG
TGTGACTTAGACTCAGAAGTGGTGGCAAAATGA

NOV8h, CG50646-03

| | | | |
|---|---|---|---|
| Protein Sequence | SEQ ID NO: 192 | 2050 aa | MW at 224498.2kD |

MAGAPPPASLPPCSLISDCCASNQRDSVGVGPSEPGVGYSLVVRRFLSRSEKRNIRVGVTRFSSYTLA
GLDTIECLADGKWSRSDQQCLAVSCDEPPIVDHASPETAHRLFGDIAFYYCSDGYSLADNSQLLCNAQ
GKWVPPEGQDMPRCIAHFCEKPPSVSYSILESVSKAKFAAGSVVSFKCMEGFVLNTSAKIECMRGGQW
NPSPMSIQCIPVRCGEPPSIMNGYASGSNYSFGAMVAYSCNKGFYIKGEKKSTCEATGQWSSPIPTCH
PVSCGEPPKVENGFLEHTTGRIFESEVRYQCNPGYKSVGSPVFVCQANRHWHSESPLMCVPLDCGKPP
PIQNGFMKGENFEVGSKGQFFCNEGLXSFVGDSSWTCQKSGKWNKKSNPKCMPAKCPEPPLLENQLVL
KELTTEVGVVTFSCKERHVLQGPSVLKCLPSQQWNDSFPVCKIVLCTPPPLISFGVPIPSSALHFGST
VKYSCVGGFFLRGNSTTLCQPDGTWSSPLPECVPVECPQPEEIPNGIIDVQGLAYLSTALYTCKPGFE
LVGNTTTLCGENGHWLGGKPTCKAIECLKPKEILNGKFSYTDLHYGQTVTYSCNRGFRLEGPSALTCL
ETGDWDVDAPSCNAIHCDSPQPIENGFVEGADYSYGAIIIYSCFPGFQVAGHAMQTCEESGWSSSIPT
CMPIDCGLPPHIDFGDCTKLKDDQGYFEQEDDMMEVPYVTPHPPYHLGAVAKTWENTKESPATHSSNF
LYGTMVSYTCNPGYELLGNPVLICQEDGTWNGSAPSCISIECDLPTAPENGFLRFTETSMGSAVQYSC
KPGHILAGSDLRICLENRKWSGASPRCEAISCKKPNPVMNGSIKGSNYTYLSTLYYECDPGYVLNGTE
RRTCQDDKNWDEDEPICIPVDCSSPPVSANGQVRGDEYTFQKEIEYTCNEGFLLEGARSRVCLANGSW
SGATPDCVPVRCATPPQLANGVTEGLDYGFMKEVTFHCHEGYILHGAPKLTCQSDGNWDAEIPLCKPV
NCGPPEDLAHGFPNGFSFIHGGHIQYQCFPGYKLHGNSSRRCLSNGSWSGSSPSCLPCRCSTPVIEYG
TVNGTDFDCGKAARIQCFKGFKLLGLSEITCEADGQWSSGFPHCEHTSCGSLPMIPNAFISETSSWKE
NVITYSCRSGYVIQGSSDLICTEKGVWSQPYPVCEPLSCGSPPSVANAVATGEAHTYESEVKLRCLEG
YTMDTDTDTFTCQKDGRWFPERISCSPKKCPLPENITHILVHGDDFSVNRQVSVSCAEGYTFEGVNIS
VCQLDGTWEPPFSDESCSPVSCGKPESPEHGFVVGSKYTFESTIIYQCEPGYELENLAVNPSGPGLFL
VDRTLSCRSELARGPIQTLFAWVSAAEGAEQRILVNRKCCCLIIPLEVLSQRNTRPCEVSVRPYWGGN
RERVCQENRQWSGGVAICKETRCETPLEFLNGKADIENRTTGPNVVYSCNRGYSLEGPSEAHCTENGT
WSHPVPLCKPNPCPVPFVIPENALLSEKEFYVDQNVSIKCREGFLLQGHGIITCNPDETWTQTSAKCE
RRYTQQPKSLNFQLAAYCSIRMFILRGGVQDGQLETAVAGASHREEQKQKREKARWYNGPPGSHMGQA
ELPPPAKGGGPPCGNFSNSSQGFMNRPLISLRWSPWGSMWPWSPQISRLSPSPAGSEESRQAGLVGFP
TAQFTCSAKGQLERFVKRVPDPMPPDWDETPPQQGSRMRPPHNRGHQTPYTRVFLLASGQCPSGTELP
EERAGSHLCCSAASAGKAQNWAEARIDELKEVGFRYWVIMKFAELKEHVLNQCKDAKNQDKTLQDPLT
RITSLERNVNDLMELKNTTRELHNATTKQGQHSSSGNPENPSKILHEKINPKTHNPQVLQEISCGPPA
HVENAIARGVHYQYGDMITYSCYSGYMLEGFLRSVCLENGTWTSPPICRAVCRFPCQNGGICQRPNAC
SCPEGWMGRLCEEPICILPCLNGGRCVAPYQCDCPPGWTGSRCHTGRPLSWFVFLVAQAHETPEDIEE
CDLDSEVVAK

| | | |
|---|---|---|
| NOV8i, CG50646-05 | SEQ ID NO: 193 | 11152 bp |

| | | |
|---|---|---|
| DNA Sequence | ORF Start: ATG at 77 | ORF Stop: TAA at 10781 |

CAATTGGTCTAGGGTCTCCCCCATTGGAATATCCATCAGTGATGAGAAATACAACGTTTGTTGAGTTT
TCTCTAGCATGAGAAGAATTTGCGCGGCTTGCTGGGGTCTGGCGCTCGTTTCGGGCTGGGCGACCTTT
CAGCAGATGTCCCCGTCGCGCAATTTCAGCTTCCGCCTCTTCCCCGAGACCGCGCCCGGGGCCCCCGG
GAGTATCCCCGCGCCGCCCGCTCCTGGCGACGAAGCGGCGGGGAGCAGAGTGGAGCGGCTGGGCCAGG
CGTTCCGCGTGCGGCTGCTGCGGGAGCTCAGCGAGCGCCTGGAGCTTGTCTTCCTGGTGGATGATTCG
TCCAGCGTGGGCGAAGTCAACTTCCGCAGCGAGCTCATGTTCGTCCGCAAGCTGCTGTCCGACTTCCC
CGTGGTGCCCACGGCCACGCGCGTGGCCATCGTGACCTTCTCGTCCAAGAACTACGTGGTGCCGCGCG
TCGATTACATCTCCACCCGCCGCGCGCGCCAGCACAAGTGCGCGCTGCTCCTCCAAGAGATCCCTGCC
ATCTCCTACCGAGGTGGCGGCACCTACACCAAGGGCGCCTTCCAGCAAGCCGCGCAAATTCTTCTTCA
TGCTAGAGAAAACTCAACAAAAGTTGTATTTCTCATCACTGATGGATATTCCAATGGGGGAGACCCTA
GACCAATTGCAGCGTCACTGCGAGATTCAGGAGTGGAGATCTTCACTTTTGGCATATGGCAAGGGAAC
ATTCGAGAGCTGAATGACATGGCTTCCACCCCAAAGGAGGAGCACTGTTACCTGCTACACAGTTTTGA
AGAATTTGAGGCTTTAGCTCGCCGGGCATTGCATGAAGATCTACCTTCTGGGAGTTTTATTCAAGATG
ATATGGTCCACTGCTCATATCTTTGTGATGAGGGCAAGGACTGCTGTGACCGAATGGGAAGCTGCAAA
TGTGGGACACACACAGGCCATTTTGAGTGCATCTGTGAAAAGGGGTATTACGGGAAAGGTCTGCAGTA
TGAATGCACAGCTTGCCCATCGGGGACATACAAACCTGAAGCCTCACCAGGAGGAATCAGCAGTTGCA
TTCCATGTCCCGATGAAAATCACACCTCTCCACCTGGAAGCACATCCCCTGAAGACTGTGTCTGCAGA
GAGGGATACAGGGCATCTGGCCAGACCTGTGAACTTGTCCACTGCCCTGCCCTGAAGCCTCCCGAAAA
TGGTTACTTTATCCAAAACACTTGCAACAACCACTTCAATGCAGCCTGTGGGGTCCGATGTCACCCTG
GATTTGATCTTGTGGGAAGCAGCATCATCTTATGTCTACCCAATGGTTTGTGGTCCGGTTCAGAGAGC
TACTGCAGAGTAAGAACATGTCCTCATCTCCGCCAGCCGAAACATGGCCACATCAGCTGTTCTACAAG
GGAAATGTTATATAAGACAACATGTTTGGTTGCCTGTGATGAAGGGTACAGACTAGAAGGCAGTGATA
AGCTTACTTGTCAAGGAAACAGCCAGTGGGATGGGCCAGAACCCCGGTGTGTGGAGCGCCACTGTTCC
ACCTTTCAGATGCCCAAAGATGTCATCATATCCCCCCACAACTGTGGCAAGCAGCCAGCCAAATTTGG
GACGATCTGCTATGTAAGTTGCCGCCAAGGGTTCATTTTATCTGGAGTCAAAGAAATGCTGAGATGTA
CCACTTCTGGAAAATGGAATGTCGGAGTTCAGGCAGCTGTGTGTAAAGACGTGGAGGCTCCTCAAATC
AACTGTCCTAAGGACATAGAGGCTAAGACTCTGGAACAGCAAGATTCTGCCAATGTTACCTGGCAGAT
TCCAACAGCTAAAGACAACTCTGGTGAAAAGGTGTCAGTCCGCGTTCATCCAGCTTTCACCCCACCTT
ACCTTTTCCCAATTGGAGATGTTGCTATCGTATACACGGCAACTGACCTATCCGGCAACCAGGCCAGC
TGCATTTTCCATATCAAGGTTATTGATGCAGAACCACCTGTCATAGACTGGTGCAGATCTCCACCTCC
CGTCCAGGTCTCGGAGAAGGTACATGCCGCAAGCTGGGATGAGCCTCAGTTCTCAGACAACTCAGGGG
CTGAATTGGTCATTACCAGAAGTCATACACAAGGAGACCTTTTCCCTCAAGGGGAGACTATAGTACAG
TATACAGCCACTGACCCCTCAGGTAATAACAGGATATGTGATATCCATATTGTCATGAAAGGTTCTCC
CTGTGAAATTCCATTCACACCTGTAAATGGGGATTTTATATGCACTCCAGATAATACTGGAGTCAACT
GTACATTAACTTGCTTGGAGGGCTACGATTTCACAGAAGGGTCTACTGACAAGTATTATTGTGCTTAT
GAAGATGGCGTCTGGAAACCAACATATACCACTGAATGGCCAGACTGTGCCAAAAAACGTTTTGCAAA
CCACGGGTTCAAGTCCTTTGAGATGTTCTACAAAGCAGCTCGTTGTGATGACTCAGATCTGATGAAGA
AGTTTTCTGAAGCATTTGAGACGACCCTGGGAAAAATGGTCCCATCATTTTGTAGTGATGCAGAGGAC
ATTGACTGCAGACTGGAGGAGAACCTGACCAAAAAATATTGCCTAGAATATAATTATGACTATGAAAA
TGGCTTTGCAATTGGTCCAGGTGGCTGGGGTGCAGCTAATAGGCTGGATTACTCTTACGATGACTTCC
TGGACACTGTGCAAGAAACAGCCACAAGCATCGGCAATGCCAAGTCCTCACGGATTAAAAGAAGTGCC
CCATTATCTGACTATAAAATTAAGTTAATTTTTAACATCACAGCTAGTGTGCCATTACCCGATGAAAG
AAATGATACCCTTGAATGGGAAAATCAGCAACGACTCCTTCAGACATTGGAAACTATCACAAATAAAC
TGAAAAGGACTCTCAACAAAGACCCCATGTATTCCTTTCAGCTTGCATCAGAAATACTTATAGCCGAC
AGCAATTCATTAGAAACAAAAAAGGCTTCCCCCTTCTGCAGACCAGGCTCAGTGCTGAGAGGGCGTAT
GTGTGTCAATTGCCCTTTGGGAACCTATTATAATCTGGAACATTTCACCTGTGAAAGCTGCCGGATCG
GATCCTATCAAGATGAAGAAGGGCAACTTGAGTGCAAGCTTTGCCCCTCTGGGATGTACACGGAATAT
ATCCATTCAAGAAACATCTCTGATTGTAAAGCTCAGTGTAAACAAGGCACCTACTCATACAGTGGACT
TGAGACTTGTGAATCGTGTCCACTGGGCACTTATCAGCCAAAATTTGGTTCCCGGAGCTGCCTCTCGT
GTCCAGAAAACACCTCAACTGTGAAAAGAGGAGCCGTGAACATTTCTGCATGTGGAGTTCCTTGTCCA
GAAGGAAAATTCTCGCGTTCTGGGTTAATGCCCTGTCACCCATGTCCTCGTGACTATTACCAACCTAA
TGCAGGGAAGGCCTTCTGCCTGGCCTGTCCCTTTTATGGAACTACCCCATTCGCTGGTTCCAGATCCA
TCACAGAATGTTCAAGTTTTAGTTCAACTTTCTCAGCGGCAGAGGAAAGTGTGGTGCCCCCTGCCTCT
CTTGGACATATTAAAAAGAGGCATGAAATCAGCAGTCAGGCAAGTCATGAATGCTTCTTTAACCCTTG
CCACAATAGTGGAACCTGCCAGCAACTTGGGCGTGGTTATGTTTGTCTCTGTCCACTTGGATATACAG
GTTTAAAGTGTGAAACAGACATCGATGAGTGCAGCCCACTGCCTTGCCTCAACAATGGAGTTTGTAAA
GACCTAGTTGGGGAATTCATTTGTGAGTGCCCATCAGGTTACACAGGTAAGCACTGTGAATTGAACAT
CAATGAATGTCAGTCTAATCCATGTAGAAATCAGGCCACCTGTGTGGATGAATTAAATTCATACAGTT
GTAAATGTCAGCCAGGATTTTCAGGCAAAAGGTGTGAAACAGGTATGTATCAACTCAGTGTTATTAAT
AACCTTAATAATGCAGTCTGTGAAGACCAGGTTGGGGGATTCTTGTGCAAATGCCCACCTGGATTTTT
GGGTACCCGATGTGGAAAGAACGTCGATGAGTGTCTCAGTCAGCCATGCAAAAATGGAGCTACCTGTA
AAGACGGTGCCAATAGCTTCAGGTGCCTGTGTGCAGCTGGCTTCACAGGATCACACTGTGAATTGAAC
ATCAATGAATGTCAGTCTAATCCATGTAGAAATCAGGCCACCTGTGTGGATGAATTAAATTCATACAG
TTGTAAATGTCAGCCAGGATTTTCAGGCAAAAGGTGTGAAACAGAACAGTCTACAGGCTTTAACCTGG
ATTTTGAAGTTTCTGGCATCTATGGATATGTCATGCTAGATGGCATGCTCCCATCTCTCCATGCTCTA
ACCTGTACCTTCTGGATGAAATCCTCTGACGACATGAACTATGGAACACCAATCTCCTATGCAGTTGA
TAACGGCAGCGACAATACCTTGCTCCTGACTGATTATAACGGGTGGGTTCTTTATGTGAATGGCAGGG
AAAAGATAACAAACTGTCCCTCGGTGAATGATGGCAGATGGCATCATATTGCAATCACTTGGACAAGT
ACTGGTGGAGCCTGGAGGGTCTATATAAATGGGGAATTATCTGACGGTGGTACTGGCCTCTCCATTGG
CAAAGCCATACCTGGTGGCGGTGCATTAGTTCTTGGGCAAGAGCAAGACAAAAAAGGAGAGGGGTTCA
ACCCGGCTGAGTCTTTTGTGGGCTCCATAAGCCAGCTCAACCTCTGGGACTATGTCCTGTCTCCACAG
CAGGTGAAGTCACTGGCTACCTCCTGCCCAGAGGAACTCAGTAAAGGAAACGTGTTAGCATGGCCTGA
TTTCTTGTCAGGAATTGTGGGGAAAGTGAAGATCGATTCTAAGAGCATATTTTGTTCTGATTGCCCAC
GCTTGGGAGGGTCAGTGCCTCATCTGAGAACTGCATCTGAAGATTTAAAACCAGGTTCCAAAGTCAAT
CTGTTCTGTGAACCAGGCTTCCAGCTGGTCGGGAACCCTGTGCAGTACTGTCTGAATCAAGGACAGTG
GACACAACCACTCCCCCACTGTGAACGCATTCGCTGTGGGGTGCCACCTCCTTTGGAGAATGGCTTCC
ATTCAGCCGATGACTTCTATGCTGGCAGCACAGTAACCTACCAGTGCAACAATGGCTACTATCTATTG
GGTGACTCAAGGATGTTCTGTACAGATAATGGGAGCTGGAACGGCGTTTCACCATCCTGCTTAGATGT
CGATGAGTGTGCAGTTGGATCAGATTGTAGTGAGCATGCTTCTTGCCTGAACGTAGATGGATCCTACA
TATGTTCATGTGTCCCACCGTACACAGGAGATGGGAAAAACTGTGCAGAACCTATAAAATGTAAGGCT
CCAGGAAATCCGGAAAATGGCCACTCCTCAGGTGAGATTTATACAGTAGGTGCCGAAGTCACATTTTC
GTGTCAGGAAGGATACCAGTTGATGGGAGTAACCAAAATCACATGTTTGGAGTCTGGAGAATGGAATC
ATCTAATACCATATTGTAAAGCTGTTTCATGTGGTAAACCGGCTATTCCAGAAAATGGTTGCATTGAG
GAGTTAGCATTTACTTTTGGCAGCAAAGTGACATATAGCTGTAATAAAGGATATACTCTGGCCCGTGA
TAAAGAATCATCCTGTCTTGCTAACAGTTCTTGGAGTCATTCCCCTCCTGTGTGTGAACCAGTGAAGT
GTTCTAGTCCGGAAAATATAAATAATGGAAAATATATTTTGAGTGGGCTTACCTACCTTTCTACTGCA
TCATATTCATGCGATACAGGATACAGCTTACAGGGCCCTTCCATTATTGAATGCACGGCTTCTGGCAT
CTGGGACAGAGCGCCACCTGCCTGTCACCTCGTCTTCTGTGGAGAACCACCTGCCATCAAAGATGCTG
TCATTACGGGGAATAACTTCACTTTCAGGAACACCGTCACTTACACTTGCAAAGAAGGCTATACTCTT
GCTGGTCTTGACACCATTGAATGCCTGGCCGACGGCAAGTGGAGTAGAAGTGACCAGCAGTGCCTGGC
TGTCTCCTGTGATGAGCCACCCATTGTGGACCACGCCTCTCCAGAGACTGCCCATCGGCTCTTTGGAG
ACATTGCATTCTACTACTGCTCTGATGGTTACAGCCTAGCAGACAATTCCCAGCTTCTCTGCAATGCC
CAGGGCAAGTGGGTACCCCCAGAAGGTCAAGACATGCCCCGTTGTATAGCTCATTTCTGTGAAAAACC
TCCATCGGTTTCCTATAGCATCTTGGAATCTGTGAGCAAAGCAAAATTTGCAGCTGGCTCAGTTGTGA
GCTTTAAATGCATGGAAGGCTTTGTACTGAACACCTCAGCAAAGATTGAATGTATGAGAGGTGGGCAG
TGGAACCCTTCCCCCATGTCCATCCAGTGCATCCCTGTGCGGTGTGGAGAGCCACCAAGCATCATGAA
TGGCTATGCAAGTGGATCAAACTACAGTTTTGGAGCCATGGTGGCTTACAGCTGCAACAAGGGGTTCT
ACATCAAAGGGGAAAAGAAGAGCACCTGCGAAGCCACAGGGCAGTGGAGTAGTCCTATACCGACGTGC
CACCCGGTATCTTGTGGTGAACCACCTAAGGTTGAGAATGGCTTTCTGGAGCATACAACTGGCAGGAT
CTTTGAGAGTGAAGTGAGGTATCAGTGTAACCCGGGCTATAAGTCAGTCGGAAGTCCTGTATTTGTCT
GCCAAGCCAATCGCCACTGGCACAGTGAATCCCCTCTGATGTGTGTTCCTCTCGACTGTGGAAAACCT
CCCCCGATCCAGAATGGCTTCATGAAAGGAGAAAACTTTGAAGTAGGGTCCAAGGTTCAGTTTTTCTG
TAATGAGGGTTATGAGCTTGTTGGTGACAGTTCTTGGACATGTCAGAAATCTGGCAAATGGAATAAGA
AGTCAAATCCAAAGTGCATGCCTGCCAAGTGCCCAGAGCCGCCCCTCTTGGAAAACCAGCTAGTATTA
AAGGAGTTGACCACCGAGGTAGGAGTTGTGACATTTTCCTGTAAAGAAGGGCATGTCCTGCAAGGCCC
CTCTGTCCTGAAATGCTTGCCATCCCAGCAATGGAATGACTCTTTCCCTGTTTGTAAGATTGTTCTTT
GTACCCCACCTCCCCTAATTTCCTTTGGTGTCCCCATTCCTTCTTCTGCTCTTCATTTTGGAAGTACT
GTCAAGTATTCTTGTGTAGGTGGGTTTTTCCTAAGAGGAAATTCTACCACCCTCTGCCAACCTGATGG
CACCTGGAGCTCTCCACTGCCAGAATGTGTTCCAGTAGAATGTCCCCAACCTGAGGAAATCCCCAATG
GAATCATTGATGTGCAAGGCCTTGCCTATCTCAGCACAGCTCTCTATACCTGCAAGCCAGGCTTTGAA
TTGGTGGGAAATACTACCACCCTTTGTGGAGAAAATGGTCACTGGCTTGGAGGAAAACCAACATGTAA
AGCCATTGAGTGCCTGAAACCCAAGGAGATTTTGAATGGCAAATTCTCTTACACGGACCTACACTATG
GACAGACCGTTACCTACTCTTGCAACCGAGGCTTTCGGCTCGAAGGTCCCAGTGCCTTGACCTGTTTA
GAGACAGGTGATTGGGATGTAGATGCCCCATCTTGCAATGCCATCCACTGTGATTCCCCACAACCCAT
TGAAAATGGTTTTGTAGAAGGTGCAGATTACAGCTATGGTGCCATAATCATCTACAGTTGCTTCCCTG
GGTTTCAGGTGGCTGGTCATGCCATGCAGACCTGTGAAGAGTCAGGATGGTCAAGTTCCATCCCAACA
TGTATGCCAATAGACTGTGGCCTCCCTCCTCATATAGATTTTGGAGACTGTACTAAACTCAAAGATGA
CCAGGGATATTTTGAGCAAGAAGACGACATGATGGAAGTTCCATATGTGACTCCTCACCCTCCTTATC
ATTTGGGAGCAGTGGCTAAAACCTGGGAAAATACAAAGGAGTCTCCTGCTACACATTCATCAAACTTT
CTGTATGGTACCATGGTTTCATACACCTGTAATCCAGGATATGAACTTCTGGGGAACCCTGTGCTGAT
CTGCTCCTGAAAATGGCTTTTTGCGTTTTACAGAGACTAGCATGGGAAGTGCTGTGCAGTATAGCTGT
AAACCTGGACACATTCTAGCAGGCTCTGACTTAAGGCTTTGTCTAGAGAATAGAAAGTGGAGTGGTGC
CTCCCCACGCTGTGAAGCCATTTCATGCAAAAAGCCAAATCCAGTCATGAATGGATCCATCAAAGGAA
AGGAGAACATGCCAGGATGACAAAAACTGGGATGAGGATGAGCCCATTTGCATTCCTGTGGACTGCAG
TTCACCCCCAGTCTCAGCCAATGGCCAGGTGAGAGGAGACGAGTACACATTCCAAAAAGAGATTGAAT
ACACTTGCAATGAAGGGTTCTTGCTTGAGGGAGCCAGGAGTCGGGTTTGTCTTGCCAATGGAAGTTGG
GGAAGGCCTGGACTATGGCTTCATGAAGGAAGTAACATTCCACTGTCACGAGGGCTACATCTTGCACG
GTGCTCCAAACTCACCTGTCAGTCAGATGGCCAACTGGGATGCAGAGATTCCTCTCTGTAAACCAGTC
AACTGTGGACCTCCTGAAGATCTTGCCCATGGTTTCCCTAATGGTTTTTCCTTTATTCATGGGGGCCA
TATACAGTATCAGTGCTTTCCTGGTTATAAGCTCCATGGAAATTCATCAAGAAGGTGCCTCTCCAATG
GCTCCTGGAGTGGCAGCTCACCTTCCTGCCTGCCTTGCAGATGTTCCACACCAGTAATTGAATATGGA
ACTGTCAATGGGACAGATTTTGACTGTGGAAAGGCAGCCCGGATTCAGTGCTTCAAAGGCTTCAAGCT
CCTAGGACTTTCTGAAATCACCTGTGAAGCCGATGGCCAGTGGAGCTCTGGGTTCCCCCACTGTGAAC
ACACTTCTTGTGGTTCTCTTCCAATGATACCAAATGCGTTCATCAGTGAGACCAGCTCTTGGAAGGAA
AATGTGATAACTTACAGCTGCAGGTCTGGATATGTCATACAAGGCAGTTCAGATCTGATTTGTACAGA
GAAAGGGGTATGGAGCCAGCCTTATCCAGTCTGTGAGCCCTTGTCCTGTGGGTCCCCACCGTCTGTCG
CCAATGCAGTGGCAACTGGAGAGGCACACACCTATGAAAGTGAAGTGAAACTCAGATGTCTGGAAGGT
TATACGATGGATACAGATACAGATACATTCACCTGTCAGAAAGATGGTCGCTGGTTCCCTGAGAGAAT
CTCCTGCAGTCCTAAAAAATGTCCTCTCCCGGAAAACATAACACATATACTTGTACATGGGGACGATT
TCAGTGTGAATAGGCAAGTTTCTGTGTCATGTGCAGAAGGGTATACCTTTGAGGGAGTTAACATATCA
GTATGTCAGCTTGATGGAACCTGGGAGCCACCATTCTCCGATGAATCTTGCAGTCCAGTTTCTTGTGG
GAAACCTGAAAGTCCAGAACATGGATTTGTGGTTGGCAGTAAATACACCTTTGAAAGCACAATTATTT
AGTGGAGGGGTGGCAATATGCAAAGAGACCAGGTGTGAAACTCCACTTGAATTTCTCAATGGGAAAGC
GGCCATCTGAGGCACACTGCACAGAAATGGAACCTGGAGCCACCCAGTCCCTCTCTGCAAACCAAAAT
CCATGCCCTGTTCCTTTTGTGATTCCCGAGAATGCTCTGCTGTCTGAAAAGGAGTTTTATGTTGATCA
AATGCAATTGCTCGAGGCGTACATTATCAATATGGAGACATGATCACCTACTCATGTTACAGTGGATA
CATGTTGGAGGGTTTCCTGAGGAGTGTTTGTTTAGAAAATGGAACATGGACATCACCTCCTATTTGCA
GAGCTGTCTGTCGATTTCCATGTCAGAATGGGGGCATCTGCCAACGCCCAAATGCTTGTTCCTGTCCA
GAGGGCTGGATGGGGCGCCTCTGTGAAGAACCAATCTGCATTCTTCCCTGTCTGAACGGAGGTCGCTG
TGTGGCCCCTTACCAGTGTGACTGCCCGCCTGGCTGGACGGGGTCTCGCTGTCATACAGCTGTTTGCC
AGTCTCCCTGCTTAAATGGTGGAAAATGTGTAAGACCAAACCGATGTCACTGTCTTTCTTCTTGGACG
GGACATAACTGTTCCAGGAAAAGGAGGACTGGGTTTTAACCACTGCACGACCATCTGGCTCTCCCAAA
AGCAGGATCATCTCTCCTCGGTAGTGCCTGGGCATCCTGGAACTTATGCAAAGAAAGTCCAACATGGT
GCTGGGTCTTGTTTAGTAAACTTGTTACTTGGGGTTACTTTTTTTATTTTGTGATATATTTTGTTATT
CCTTGTGACATACTTTCTTACATGTTTCCATTTTTAAATATGCCTGTATTTTCTATATAAAAATTATA
TTAAATAGATGCTGCTCTACCCTCACAAAATGTACATATTCTGCTGTCTATTGGGAAAGTTCCTGGTA
CACATTTTTATTCAGTTACTTAAAATGATTTTTCCATTAAAGTATATTTTGCTACTAAATAAAAAAAA

NOV8i, CG50646-05

| | | | |
|---|---|---|---|
| Protein Sequence | SEQ ID NO: 194 | 3568 aa | MW at 389944.3kD |

MPRICAACWGLALVSGWATFQQMSPSRNFSFRLFPETAPGAPGSIPAPPAPGDEAAGSRVERLGQAFR
VRLLRELSERLELVFLVDDSSSVGEVNFRSELMFVRKLLSDFPVVPTATRVAIVTFSSKNYVVPRVDY
ISTRRARQHKCALLLQEIPAISYRGGGTYTKGAFQQAAQILLHARENSTKVVFLITDGYSNGGDPRPI
AASLRDSGVEIFTFGIWQGNIRELNDMASTPKEEHCYLLHSFEEFEALARRALHEDLPSGSFIQDDMV
HCSYLCDEGKDCCDRMGSCKCGTHTGHFECICEKGYYGKGLQYECTACPSGTYKPEASPGGISSCIPC
PDENHTSPPGSTSPEDCVCREGYRASGQTCELVHCPALKPPENGYFIQNTCNNHFNAACGVRCHPGFD
LVGSSIILCLPNGLWSGSESYCRVRTCPHLRQPKHGHISCSTREMLYKTTCLVACDEGYPLEGSDKLT
CQGNSQWDGPEPRCVERHCSTFQMPKDVIISPHNCGKQPAKFGTICYVSCRQGFILSGVKEMLRCTTS
GKWNVGVQAAVCKDVEAPQINCPKDIEAKTLEQQDSANVTWQIPTAKDNSGEKVSVRVHPAFTPPYLF
PIGDVAIVYTATDLSGNQASCIFRIKVIDAEPPVIDWCRSPPPVQVSEKVHAASWDEPQFSDNSGAEL
VITRSHTQGDLFPQGETIVQYTATDPSGNNRICDIHIVMKGSPCEIPFTPVNGDFICTPDNTGVNCTL
TCLEGYDFTEGSTDKYYCAYEDGVWKPTYTTEWPDCAKKRFANHGFKSFEMFYKAARCDDSDLMKKFS
EAFETTLGKMVPSFCSDAEDIDCRLEENLTKKYCLEYNYDYENGFAIGPGGWGAANRLDYSYDDFLDT
VQETATSIGNAKSSRIKRSAPLSDYKIKLIFNITASVPLPDERNDTLEWENQQRLLQTLETITNKLKR
TLNKDPMYSFQLASEILIADSNSLETKKASPFCRPGSVLRGRMCVNCPLGTYYNLEHFTCESCRIGSY
QDEEGQLECKLCPSGMYTEYIRSRNISDCKAQCKQGTYSYSGLETCESCPLGTYQPKFGSRSCLSCPE
NTSTVKRGAVNISACGVPCPEGKFSRSGLMPCHPCPRDYYQPNAGKAFCLACPFYGTTPFAGSRSITE
CSSFSSTFSAAEESVVPPASLGHIKKRHEISSQASHECFFNPCHNSGTCQQLGRGYVCLCPLGYTGLK
CETDIDECSPLPCLNNGVCKDLVGEFICECPSGYTGKHCELNINECQSNPCRNQATCVDELNSYSCKC
QPGFSGKRCETGMYQLSVINNLNNAVCEDQVGGFLCKCPPGFLGTRCGKNVDECLSQPCKNGATCKDG
ANSFRCLCAAGFTGSHCELNINECQSNPCRNQATCVDELNSYSCKCQPGFSGKRCETEQSTGFNLDFE
VSGIYGYVMLDGMLPSLHALTCTFWMKSSDDMNYGTPISYAVDNGSDNTLLLTDYNGWVLYVNGREKI
TNCPSVNDGRWHHIAITWTSTGGAWRVYINGELSDGGTGLSIGKAIPGGGALVLGQEQDKKGEGFNPA
ESFVGSISQLNLWDYVLSPQQVKSLATSCPEELSKGNVLAWPDFLSGIVGKVKIDSKSIFCSDCPRLG
GSVPHLRTASEDLKPGSKVNLFCEPGFQLVGNPVQYCLNQGQWTQPLPHCERIRCGVPPPLENGFHSA
DDFYAGSTVTYQCNNGYYLLGDSRMFCTDNGSWNGVSPSCLDVDECAVGSDCSEHASCLNVDGSYICS
CVPPYTGDGKNCAEPIKCKAPGNPENGHSSGEIYTVGAEVTFSCQEGYQLMGVTKITCLESGEWNHLI
PYCKAVSCGKPAIPENGCIEELAFTFGSKVTYRCNKGYTLAGDKESSCLANSSWSHSPPVCEPVKCSS
PENINNGKYILSGLTYLSTASYSCDTGYSLQGPSIIECTASGIWDRAPPACHLVFCGEPPAIKDAVIT
GNNFTFRNTVTYTCKEGYTLAGLDTIECLADGKWSRSDQQCLAVSCDEPPIVDHASPETAHRLFGDIA
FYYCSDGYSLADNSQLLCNAQGKWVPPEGQDMPRCIAHFCEKPPSVSYSILESVSKAKFAAGSVVSFK
CMEGFVLNTSAKIECMRGGQWNPSPMSIQCIPVRCGEPPSIMNGYASGSNYSFGAMVAYSCNKGFYIK
GEKKSTCEATGQWSSPIPTCHPVSCGEPPKVENGFLEHTTGRIFESEVRYQCNPGYKSVGSPVFVCQA
NRHWHSESPLMCVPLDCGKPPPIQNGFMKGENFEVGSKVQFFCNEGYELVGDSSWTCQKSGKWNKKSN
PKCMPAKCPEPPLLENQLVLKELTTEVGVVTFSCKEGHVLQGPSVLKCLPSQQWNDSFPVCKIVLCTP
PPLISFGVPIPSSALHFGSTVKYSCVGGFFLRGNSTTLCQPDGTWSSPLPECVPVECPQPEEIPNGII
DVQGLAYLSTALYTCKPGFELVGNTTTLCGENGHWLGGKPTCKAIECLKPKEILNGKFSYTDLHYGQT
VTYSCNRGFRLEGPSALTCLETGDWDVDAPSCNAIHCDSPQPIENGFVEGADYSYGAIIIYSCFPGFQ
VAGHAMQTCEESGWSSSIPTCMPIDCGLPPHIDFGDCTKLKDDQGYFEQEDDMMEVPYVTPHPPYHLG
AVAKTWENTKESPATHSSNFLYGTMVSYTCNPGYELLGNPVLICQEDGTWNGSAPSCISIECDLPTAP
ENGFLRFTETSMGSAVQYSCKPGHILAGSDLRLCLENRKWSGASPRCEAISCKKPNPVMNGSIKGSNY
TYLSTLYYECDPGYVLNGTERRTCQDDKNWDEDEPICIPVDCSSPPVSANGQVRGDEYTFQKEIEYTC
NEGFLLEGARSRVCLANGSWSGATPDCVPVRCATPPQLANGVTEGLDYGFMKEVTFHCHEGYILRGAP
KLTCQSDGNWDAEIPLCKPVNCGPPEDLAHGFPNGFSFIHGGHIQYQCFPGYKLHGNSSRRCLSNGSW
SGSSPSCLPCRCSTPVIEYGTVNGTDFDCGKAARIQCFKGFKLLGLSEITCEADGQWSSGFPHCEHTS
CGSLPMIPNAFISETSSWKENVITYSCRSGYVIQGSSDLICTEKGVWSQPYPVCEPLSCGSPPSVANA
VATGEAHTYESEVKLRCLEGYTMDTDTDTFTCQKDGRWFPERISCSPKKCPLPENITHILVHGDDFSV
NRQVSVSCAEGYTFEGVNISVCQLDGTWEPPFSDESCSPVSCGKPESPEHGFVVGSKYTFESTIIYQC
EPGYELEGNRERVCQENRQWSGGVAICKETRCETPLEFLNGKADIENRTTGPNVVYSCNRGYSLEGPS
EAHCTENGTWSHPVPLCKPNPCPVPFVIPENALLSEKEFYVDQNVSIKCREGFLLQGHGIITCNPDET
WTQTSAKCEKISCGPPAHVENAIARGVHYQYGDMITYSCYSGYMLEGFLRSVCLENGTWTSPPICRAV
CRFPCQNGGICQRPNACSCPEGWMGRLCEEPICILPCLNGGRCVAPYQCDCPPGWTGSRCHTAVCQSP
CLNGGKCVRPNRCHCLSSWTGHNCSRKRRTGF

| | |
|---|---|
| SEQ ID NO: 195 | 11158 bp |

| | | |
|---|---|---|
| NOV8j, SNP13380898 of | ORF Start: ATG at 77 | ORF Stop: TAA at 10787 |

| | | |
|---|---|---|
| CG50646-04, DNA Sequence | SNP Pos: 1068 | SNP Change: G to C |

CAATTGGTCTAGGGTCTCCCCCATTGGAATATCCATCAGTGATGAGAAATACAACGTTTGTTGAGTTT
TCTCTAGCATGAGAAGAATTTGCGCGGCTTGCTGGGGTCTGGCGCTCGTTTCGGGCTGGGCGACCTTT
CAGCAGATGTCCCCGTCGCGCAATTTCAGCTTCCGCCTCTTCCCCGAGACCGCGCCCGGGGCCCCCGG
GAGTATCCCCGCGCCGCCCGCTCCTGGCGACGAAGCGGCGGGGAGCAGAGTGGAGCGGCTGGGCCAGG
CGTTCCGCGTGCGGCTGCTGCGGGAGCTCAGCGAGCGCCTGGAGCTTGTCTTCCTGGTGGATGATTCG
TCCAGCGTGGGCGAAGTCAACTTCCGCAGCGAGCTCATGTTCGTCCGCAAGCTGCTGTCCGACTTCCC
CGTGGTGCCCACGGCCACGCGCGTGGCCATCGTGACCTTCTCGTCCAAGAACTACGTGGTGCCGCGCG
TCGATTACATCTCCACCCGCCGCGCGCGCCAGCACAAGTGCGCGCTGCTCCTCCAAGAGATCCCTGCC
ATCTCCTACCGAGGTGGCGGCACCTACACCAAGGGCGCCTTCCAGCAAGCCGCGCAAATTCTTCTTCA
TGCTAGAGAAAACTCAACAAAAGTTGTATTTCTCATCACTGATGGATATTCCAATGGGGGAGACCCTA
GACCAATTGCAGCGTCACTGCGAGATTCAGGAGTGGAGATCTTCACTTTTGGCATATGGCAAGGGAAC
ATTCGAGAGCTGAATGACATGGCTTCCACCCCAAAGGAGGAGCACTGTTACCTGCTACACAGTTTTGA
AGAATTTGAGGCTTTAGTCGCCCTCTGTCATATGTTATTTGTAGATCTACCTTCTGGGAGTTTTATTC
AAGATGATATGGTCCACTGCTCATATCTTTGTGATGAAGGCAAGGACTGCTGTGACCGAATGGGAAGC
TGCAAATGTGGGAAACACACAGGCCATTTTGAGTGCATCTGTGAAAAGGGGTATAACGGGAAAGGTCT
GCAGTATGACTGCACAGTTTGCCCATCGGGGACATACAAACCTGAAGCCTCACCAGGAGGAATCAGCA
GTTGCATTCCATGTCCTGATGAAAATCACACCTCTCCACCTGGAAGCACATCCCCTGAAGACTGTGTC
TGCAGAGAGGGATACAGGGCATCTGGCCAGACCTGTGAAGTTGTCCACTGCCCTGCCCTGAAGCCTCC
CGAAAATGGTTACTTTATCCAAAACACTTGCAACAACCACTTCAATGCAGCCTGTGGGGTCCGATGTC
ACCCTGGATTTGATCTTGTGGGAAGCAGCATCATCTTATGTCTACCCAATGGTTTGTGGTCCGGTTCA
GAGAGCTACTGCAGAGTAAGAACATGTCCTCATCTCCGCCAGCCGAAACATGGCCACATCAGCTGTTC
TACAAGGGAAATGTTATATAAGACAACATGTTTGGTTGCCTGTGATGAAGGGTACAGGCTAGAAGGCA
GTGATAAGCTTACTTGTCAAGGAAACAGCCAGTGGGATGGGCCAGAACCCCGGTGTGTGGAGCGCCAC
TGTTCCACCTTTCAGATGCCCAAAGATGTCATCATATCCCCCCACAACTGTGGCAAGCAGCCAGCCAA
ATTTGGGACGATCTGCTATGTAAGTTGCCGCCAAGGGTTCATTTTATCTGGAGTCAAAGAAATGCTGA
CAAATCAACTGTCCTAAGGACATAGAGGCTAAGACTCTGGAACAGCAAGATTCTGCCAATGTTACCTG
GCAGATTCCAACAGCTAAAGACAACTCTGGTGAAAAGGTGTCAGTCCACGTTCATCCAGCTTTCACCC
CACCTTACCTTTTCCCAATTGGAGATGTTGCTATCGTATACACGGCAACTGACCTATCCGGCAACCAG
GCCAGCTGCATTTTCCATATCAAGGTTATTGATGCAGAACCACCTGTCATAGACTGGTGCAGATCTCC
ACCTCCCGTCCAGGTCTCGGAGAAGGTACATGCCGCAAGCTGGGATGAGCCTCAGTTCTCAGACAACT
CAGGTGCTGAATTGGTCATTACCAGAAGTCATACACAAGGAGACCTTTTCCCTCAAGGGGAGACTATA
GTACAGTATACAGCCACTGACCCCTCAGGCAATAACAGGACATGTGATATCCATATTGTCATAAAAGG
TTCTCCCTGTGAAATTCCATTCACACCTGTAAATGGGGATTTTATATGCACTCCAGATAATACTGGAG
TCAACTGTACATTAACTTGCTTGGAGGGCTATGATTTCACAGAAGGGTCTACTGACAAGTATTATTGT
GCTTATGAAGATGGCGTCTGGAAACCAACATATACCACTGAATGGCCAGACTGTGCCAGTAAGCGTTT
TGCAAACCACGGGTTCAAGTCCTTTGAGATGTTCTACAAAGCAGCTCGTTGTGATGACACAGATCTGA
TGAAGAAGTTTTCTGAAGCATTTGAGACGACCCTGGGAAAAATGGTCCCATCATTTTGTAGTGATGCA
GAGGACATTGACTGCAGACTGGAGGAGAACCTGACCAAAAAATATTGCCTAGAATATAATTATGACTA
TGAAAATGGCTTTGCAATTGGTCCAGGTGGCTGGGGTGCAGCTAATAGGCTGGATTACTCTTACGATG
ACTTCCTGGACACTGTGCAAGAAACAGCCACAAGCATCGGCAATGCCAAGTCCTCACGGATTAAAAGA
AGTGCCCCATTATCTGACTATAAAATTAAGTTAATTTTTAACATCACAGCTAGTGTGCCATTACCCGA
TGAAAGAAATGATACCCTTGAATGGGAAAATCAGCAACGACTCCTTCAGACATTGGAAACTATCACAA
ATAAACTGAAAAGGACTCTCAACAAAGACCCCATGTATTCCTTTCAGCTTGCATCAGAAATACTTATA
GCCGACAGCAATTCATTAGAAACAAAAAAGGCTTCCCCCTTCTGCAGACCAGGCTCAGTGCTGAGAGG
GCGTATGTGTGTCAATTGCCCTTTGGGAACCTATTATAATCTGGAACATTTCACCTGTGAAAGCTGCC
GGATCGGATCCTATCAAGATGAAGAAGGGCAACTTGAGTGCAAGCTTTGCCCCTCTGGGATGTACACG
GAATATATCCATTCAAGAAACATCTCTGATTGTAAAGCTCAGTGTAAACAAGGCACCTACTCATACAG
TGGACTTGAGACTTGTGAATCGTGTCCACTGGGCACTTATCAGCCAAAATTTGGTTCCCGGAGCTGCC
TCTCGTGTCCAGAAAACACCTCAACTGTGAAAAGAGGAGCCGTGAACATTTCTGCATGTGGAGTTCCT
TGTCCAGAAGGAAAATTCTCGCGTTCTGGGTTAATGCCCTGTCACCCATGTCCTCGTGACTATTACCA
ACCTAATGCAGGGAAGGCCTTCTGCCTGGCCTGTCCCTTTTATGGAACTACCCCATTCGCTGGTTCCA
GATCCATCACAGAATGTTCAAGTTTTAGTTCAACTTTCTCAGCGGCAGAGGAAAGTGTGGTGCCCCCT
GCCTCTCTTGGACATATTAAAAAGAGGCATGAAATCAGCAGTCAGGCAAGTCATGAATGCTTCTTTAA
CCCTTGCCACAATAGTGGAACCTGCCAGCAACTTGGGCGTGGTTATGTTTGTCTCTGTCCACTTGGAT
ATACAGGTTTAAAGTGTGAAACAGACATCGATGAGTGCAGCCCACTGCCTTGCCTCAACAATGGAGTT
TGTAAAGACCTAGTTGGGGAATTCATTTGTGAGTGCCCATCAGGTTACACAGGTAAGCACTGTGAATT
GAACATCAATGAATGTCAGTCTAATCCATGTAGAAATCAGGCCACCTGTGTGGATGAATTAAATTCAT
ACAGTTGTAAATGTCAGCCAGGATTTTCAGGCAAAAGGTGTGAAACAGGTATGTATCAACTCAGTGTT
ATTAATAACCTTAATAATGCAGTCTGTGAAGACCAGGTTGGGGGATTCTTGTGCAAATGCCCACCTGG
ATTTTTGGGTACCCGATGTGGAAAGAACGTCGATGAGTGTCTCAGTCAGCCATGCAAAAATGGAGCTA
CCTGTAAAGACGGTGCCAATAGCTTCAGGTGCCTGTGTGCAGCTGGCTTCACAGGATCACACTGTGAA
TTGAACATCAATGAATGTCAGTCTAATCCATGTAGAAATCAGGCCACCTGTGTGGATGAATTAAATTC
ATACAGTTGTAAATGTCAGCCAGGATTTTCAGGCAAAAGGTGTGAAACAGAACAGTCTACAGGCTTTA
ACCTGGATTTTGAAGTTTCTGGCATCTATGGATATGTCATGCTAGATGGCATGCTCCCATCTCTCCAT
GCTCTAACCTGTACCTTCTGGATGAAATCCTCTGACGACATGAACTATGGAACACCAATCTCCTATGC
AGTTGATAACGGCAGCGACAATACCTTGCTCCTGACTGATTATAACGGGTGGGTTCTTTATGTGAATG
GCAGGGAAAAGATAACAAACTGTCCCTCGGTGAATGATGGCAGATGGCATCATATTGCAATCACTTGG
ACAAGTACTGGTGGAGCCTGGAGGGTCTATATAAATGGGGAATTATCTGACGGTGGTACTGGCCTCTC
CATTGGCAAAGCCATACCTGGTGGCGGTGCATTAGTTCTTGGGCAAGAGCAAGACAAAAAAGGAGAGG
GGTTCAACCCGGCTGAGTCTTTTGTGGGCTCCATAAGCCAGCTCAACCTCTGGGACTATGTCCTGTCT
GCCTGATTTCTTGTCAGGAATTGTGGGGAAAGTGAAGATCGATTCTAAGAGCATATTTTGTTCTGATT
GCCCACGCTTGGGAGGGTCAGTGCCTCATCTGAGAACTGCATCTGAAGATTTAAAACCAGGTTCCAAA
GTCAATCTGTTCTGTGAACCAGGCTTCCAGCTGGTCGGGAACCCTGTGCAGTACTGTCTGAATCAAGG
ACAGTGGACACAACCACTCCCCCACTGTGAACGCATTCGCTGTGGGGTGCCACCTCCTTTGGAGAATG
GCTTCCATTCAGCCGATGACTTCTATGCTGGCAGCACAGTAACCTACCAGTGCAACAATGGCTACTAT
CTATTGGGTGACTCAAGGATGTTCTGTACAGATAATGGGAGCTGGAACGGCGTTTCACCATCCTGCTT
AGATGTCGATGAGTGTGCAGTTGGATCAGATTGTAGTGAGCATGCTTCTTGCCTGAACGTAGATGGAT
CCTACATATGTTCATGTGTCCCACCGTACACAGGAGATGGGAAAAACTGTGCAGAACCTATAAAATGT
AAGGCTCCAGGAATCCGGAAATCCGGCCACTCCTCAGGTGAGATTTATACAGTAGGTGCCGAAGTCAC
ATTTTCGTGTCAGGAAGGATACCAGTTGATGGGAGTAACCAAAATCACATGTTTGGAGTCTGGAGAAT
GGAATCATCTAATACCATATTGTAAAGCTGTTTCATGTGGTAAACCGGCTATTCCAGAAAATGGTTGC
ATTGAGGAGTTAGCATTTACTTTTGGCAGCAAAGTGACATATAGGTGTAATAAAGGATATACTCTGGC
CGGTGATAAAGAATCATCCTGTCTTGCTAACAGTTCTTGGAGTCATTCCCCTCCTGTGTGTGAACCAG
TGAAGTGTTCTAGTCCGGAAAATATAAATAATGGAAAATATATTTTGAGTGGGCTTACCTACCTTTCT
ACTGCATCATATTCATGCGATACAGGATACAGCTTACAGGGCCCTTCCATTATTGAATGCACGGCTTC
TGGCATCTGGGACAGAGCGCCACCTGCCTGTCACCTCGTCTTCTGTGGAGAACCACCTGCCATCAAAG
ATGCTGTCATTACGGGGAATAACTTCACTTTCAGGAACACCGTCACTTACACTTGCAAAGAAGGCTAT
ACTCTTGCTGGTCTTGACACCATTGAATGCCTGGCCGACGGCAAGTGGAGTAGAAGTGACCAGCAGTG
CCTGGCTGTCTCCTGTGATGAGCCACCCATTGTGGACCACGCCTCTCCAGAGACTGCCCATCGGCTCT
TTGGAGACATTGCATTCTACTACTGCTCTGATGGTTACAGCCTAGCAGACAATTCCCAGCTTCTCTGC
AATGCCCAGGGCAAGTGGGTACCCCCAGAAGGTCAAGACATGCCCCGTTGTATAGCTCATTTCTGTGA
AAAACCTCCATCGGTTTCCTATAGCATCTTGGAATCTGTGAGCAAAGCAAAATTTGCAGCTGGCTCAG
TTGTGAGCTTTAAATGCATGGAAGGCTTTGTACTGAACACCTCAGGLAAGATTGAATGTATGAGAGGT
GGGCAGTGGAACCCTTCCCCCATGTCCATCCAGTGCATCCCTGTGCGGTGTGGAGAGCCACCAAGCAT
CATGAATGGCTATGCAAGTGGATCAAACTACAGTTTTGGAGCCATGGTGGCTTACAGCTGCAACAAGG
GGTTCTACATCAAAGGGGAAAAGAAGAGCACCTGCGAAGCCACAGGGCAGTGGAGTAGTCCTATACCG
ACGTGCCACCCGGTATCTTGTGGTGAACCACCTAAGGTTGAGAATGGCTTTCTGGAGCATACAACTGG
CAGGATCTTTGAGAGTGAAGTGAGGTATCAGTGTAACCCGGGCTATAAGTCAGTCGGAAGTCCTGTAT
TTGTCTGCCAAGCCAATCGCCACTGGCACAGTGAATCCCCTCTGATGTGTGTTCCTCTCGACTGTGGA
AAACCTCCCCCGATCCAGAATGGCTTCATGAAAGGAGALAACTTTGAAGTAGGGTCCAAGGTTCAGTT
TTTCTGTAATGAGGGTTATGAGCTTGTTGGTGACAGTTCTTGGACATGTCAGAAATCTGGCAAATGGA
ATAAGAAGTCAAATCCAAAGTGCATGCCTGCCAAGTGCCCAGAGCCGCCCCTCTTGGAAAACCAGCTA
GTATTAAAGGAGTTGACCACCGAGGTAGGAGTTGTGACATTTTCCTGTAAAGAAGGGCATGTCCTGCA
AGGCCCCTCTGTCCTGAAATGCTTGCCATCCCAGCAATGGAATGACTCTTTCCCTGTTTGTAAGATTG
TTCTTTGTACCCCACCTCCCCTAATTTCCTTTGGTGTCCCCATTCCTTCTTCTGCTCTTCATTTTGGA
AGTACTGTCAAGTATTCTTGTGTAGGTGGGTTTTTCCTAAGAGGAAATTCTACCACCCTCTGCCAACC
TGATGGCACCTGGAGCTCTCCACTGCCAGAATGTGTTCCAGTAGAATGTCCCCAACCTGAGGAAATCC
CCAATGGAATCATTGATGTGCAAGGCCTTGCCTATCTCAGCACAGCTCTCTATACCTGCAAGCCAGGC
TTTGAATTGGTGGGAAATACTACCACCCTTTGTGGAGAAAATGGTCACTGGCTTGGAGGAAAACCAAC
ATGTAAAGCCATTGAGTGCCTGAAACCCAAGGAGATTTTGAATGGCAAATTCTCTTACACGGACCTAC
ACTATGGACAGACCGTTACCTACTCTTGCAACCGAGGCTTTCGGCTCGAAGGTCCCAGTGCCTTGACC
TGTTTAGAGACAGGTGATTGGGATGTAGATGCCCCATCTTGCAATGCCATCCACTGTGATTCCCCACA
ACCCATTGAAAATGGTTTTGTAGAAGGTGCAGATTACAGCTATGGTGCCATAATCATCTACAGTTGCT
TCCCTGGGTTTCAGGTGGCTGGTCATGCCATGCAGACCTGTGAAGAGTCAGGATGGTCAAGTTCCATC
CCAACATGTATGCCAATAGACTGTGGCCTCCCTCCTCATATAGATTTTGGAGACTGTACTAAACTCAA
AGATGACCAGGGATATTTTGAGCAAGAAGACGACATGATGGAAGTTCCATATGTGACTCCTCACCCTC
CTTATCATTTGGGAGCAGTGGCTAAAACCTGGGAAAATACAAAGGAGTCTCCTGCTACACATTCATCA
AACTTTCTGTATGGTACCATGGTTTCATACACCTGTAATCCAGGATATGAACTTCTGGGGAACCCTGT
GCTGATCTGCCAGGAAGATGGAACTTGGAATGGCAGTGCACCATCCTGCATTTCAATTGAATGTGACT
TGCCTACTGCTCCTGAAAATGGCTTTTTGCGTTTTACAGAGACTAGCATGGGAAGTGCTGTGCAGTAT
AGCTGTAAACCTGGACACATTCTAGCAGGCTCTGACTTAAGGCTTTGTCTAGAGAATAGAAAGTGGAG
TGGTGCCTCCCCACGCTGTGAAGCCATTTCATGCAAAAAGCCAAATCCAGTCATGAATGGATCCATCA
AAGGAAGCAACTACACATACCTGAGCACGTTGTACTATGAGTGTGACCCCGGATATGTGCTGAATGGC
ACTGAGAGGAGAACATGCCAGGATGACAAAAACTGGGATGAGGATGAGCCCATTTGCATTCCTGTGGA
CTGCAGTTCACCCCCAGTCTCAGCCAATGGCCAGGTGAGAGGAGACGAGTACACATTCCAAAAAGAGA
TTGAATACACTTGCAATGAAGGGTTCTTGCTTGAGGGAGCCAGGAGTCGGGTTTGTCTTGCCAATGGA
AGTTGGAGTGGAGCCACTCCCGACTGTGTGCCTGTCAGATGTGCCACCCCGCCACAACTGGCCAATGG
GGTGACGGAAGGCCTGGACTATGGCTTCATGAAGGAAGTAACATTCCACTGTCACGAGGGCTACATCT
TGCACGGTGCTCCAAACTCACCTGTCAGTCAGATGGCAACTGGGATGCAGAGATTCCTCTCTGTAAAA
CCAGTCAACTGTGGACCTCCTGAAGATCTTGCCCATGGTTTCCCTAATGGTTTTTCCTTTATTCATGG
GGGCCATATACAGTATCAGTGCTTTCCTGGTTATAAGCTCCATGGAAATTCATCAAGAAGGTGCCTCT
CCAATGGCTCCTGGAGTGGCAGCTCACCTTCCTGCCTGCCTTGCAGATGTTCCACACCAGTAATTGAA
TATGGAACTGTCAATGGGACAGATTTTGACTGTGGAAAGGCAGCCCGGATTCAGTGCTTCAAAGGCTT
CAAGCTCCTAGGACTTTCTGAAATCACCTGTGAAGCCGATGGCCAGTGGAGCTCTGGGTTCCCCCACT
GTGAACACACTTCTTGTGGTTCTCTTCCAATGATACCAAATGCGTTCATCAGTGAGACCAGCTCTTGG
AAGGAAAATGTGATAACTTACAGCTGCAGGTCTGGATATGTCATACAAGGCAGTTCAGATCTGATTTG
TACAGAGAAAGGGGTATGGAGCCAGCCTTATCCAGTCTGTGAGCCCTTGTCCTGTGGGTCCCCACCGT
CTGTCGCCAATGCAGTGGCAACTGGAGAGGCACACACCTATGAAAGTGAAGTGAAACTCAGATGTCTG
GAAGGTTATACGATGGATACAGATACAGATACATTCACCTGTCAGAAAGATGGTCGCTGGTTCCCTGA
GAGAATCTCCTGCAGTCCTAAAAAATGTCCTCTCCCGGAAAACATAACACATATACTTGTACATGGGG
ACGATTTCAGTGTGAATAGGCAAGTTTCTGTGTCATGTGCAGAAGGGTATACCTTTGAGGGAGTTAAC
ATATCAGTATGTCAGCTTGATGGAACCTGGGAGCACCATTCTCCGATGCAGTCTTGCAGTCCAGTTTC
TTGTGGGAAACCTGAAAGTCCAGAACATGGATTTGTGGTTGGCAGTAAATACACCTTTGAAAGCACAA
TTATTTATCAGTGTGAGCCTGGCTATGAACTAGAGGGGAACAGGGAACGTGTCTGCCAGGAGAACAGA
CAGTGGAGTGGAGGGGTGGCAATATGCAAAGAGACCAGGTGTGAAACTCCACTTGAATTTCTCAATGG
GAAAGCTGACATTGAAAACAGGACGACTGGACCCAACGTGGTATATTCCTGCAACAGAGGCTACAGTC
TTGAAGGGCCATCTGAGGCACACTGCACAGAAAATGGAACCTGGAGCCACCCAGTCCCTCTCTGCAAA
CCAAATCCATGCCCTGTTCCTTTTGTGATTCCCGAGAATGCTCTGCTGTCTGAAAAGGAGTTTTATGT
TGATCAGAATGTGTCCATCAAATGTAGGGAAGGTTTTCTGCTGCAGGGCCACGGCATCATTACCTGCA
ACCCCGACGAGACGTGGACACAGACAAGCGCCAAATGTGAAAAAATCTCATGTGGTCCACCAGCTCAC
GTAGAAAATGCAATTGCTCGAGGCGTACATTATCAATATGGAGACATGATCACCTACTCATGTTACAG
TGGATACATGTTGGAGGGTTTCCTGAGGAGTGTTTGTTTAGAAAATGGAACATGGACATCACCTCCTA
TTTGCAGAGCTGTCTGTCGATTTCCATGTCAGAATGGGGGCATCTGCCAACGCCCAAATGCTTGTTCC
TGTCCAGAGGGCTGGATGGGGCGCCTCTGTGAAGAACCAATCTGCATTCTTCCCTGTCTGAACGGAGG
TCGCTGTGTGGCCCCTTACCAGTGTGACTGCCCGCCTGGCTGGACGGGGTCTCGCTGTCATACAGCTG
TTTGCCAGTCTCCCTGCTTAAATGGTGGAAAATGTGTAAGACCAAACCGATGTCACTGTCTTTCTTCT
TGGACGGGACATAACTGTTCCAGGAAAAGGAGGACTGGGTTTTAACCACTGCACGACCATCTGGCTCT
CCCAAAAGCAGGATCATCTCTCCTCGGTAGTGCCTGGGCATCCTGGAACTTATGCAAAGAAAGTCCAA
CATGGTGCTGGGTCTTGTTTAGTAAACTTGTTACTTGGGGTTACTTTTTTTATTTTGTGATATATTTT
ATTATATTAAATAGATGCTGCTCTACCCTCACAAAATGTACATATTCTGCTGTCTATTGGGAAAGTTC
CTGGTACACATTTTTATTCAGTTACTTAAAATGATTTTTCCATTAAAGTATATTTTGCTACTAAATAA
AAAAAA

NOV8j, SNP13380898 of

| | | |
|---|---|---|
| CG50646-04, Protein | SEQ ID NO: 196 | MW at 390026.5kD |

| | | | |
|---|---|---|---|
| Sequence | SNP Pos: 331 | 3570 aa | SNP Change: Gly to Ala |

MRRICAACWGLALVSGWATFQQMSPSRNFSFRLFPETAPGAPGSIPAPPAPGDEAAGSRVERLGQAFR
VRLLRELSERLELVFLVDDSSSVGEVNFRSELMFVRKLLSDFPVVPTATRVAIVTFSSKNYVVPRVDY
ISTRRARQHKCALLLQEIPAlSYRGGGTYTKGAFQQAAQILLHARENSTKVVFLITDGYSNGGDPRPI
AASLRDSGVEIFTFGIWQGNIRELNDMASTPKEEHCYLLHSFEEFEALVALCHMLFVDLPSGSFIQDD
MVHCSYLCDEGKDCCDRMGSCKCGKHTGHFECICEKGYNGKGLQYDCTVCPSGTYKPEASPGGISSCI
PCPDENHTSPPGSTSPEDCVCREGYFASGQTCEVVHCPALKPPENGYFIQNTCNNHFNAACGVRCHPG
FDLVGSSIILCLPNGLWSGSESYCRVRTCPHLRQPKHGHISCSTREMLYKTTCLVACDEGYPLEGSDK
LTCQGNSQWDGPEPRCVERHCSTFQMPKDVIISPHNCGKQPAKFGTICYVSCRQGFILSGVKEMLRCT
TSGWNNVGVQAAVCKDVEAPQINCPKDIEAKTLEQQDSANVTWQIPTAKDNSGEKVSVHVHPAFTPPY
LFPIGDVAIVYTATDLSGNQASCIFHIKVIDAEPPVIDWCRSPPPVQVSEKVHAASWDEPQFSDNSGA
ELVITRSHTQGDLFPQGETIVQYTATDPSGNNRTCDIHIVIKGSPCEIPFTPVNGDFICTPDNTGVNC
TLTCLEGYDFTEGSTDKYYCAYEDGVWKPTYTTEWPDCASKRFANHGFKSFEMFYKAARCDDTDLMKK
FSEAFETTLGKMVPSFCSDAEDIDCRLEENLTKKYCLEYNYDYENGFAIGPGGWGAANRLDYSFDDFL
DTVQETATSIGNAKSSRIKRSAPLSDYKIKLIFNITASVPLPDERNDTLEWENQQRLLQTLETITNKL
KRTLNKDPMYSFQLASEILIADSNSLETKKASPFCRPGSVLRGRMCVNCPLGTYYNLEHFTCESCRIG
SYQDEEGQLECKLCPSGMYTEYIHSRNISDCKAQCKQGTYSYSGLETCESCPLGTYQPKFGSRSCLSC
PENTSTVKRGAVNISACGVPCPEGKFSRSGLMPCHPCPRDYYQPNAGKAFCLACPFYGTTPFAGSRSI
TECSSFSSTFSAAEESVVPPASLGHIKKRHEISSQASHECFFNPCHNSGTCQQLGRGYVCLCPLGYTG
LKCETDIDECSPLPCLNNGVCKDLVGEFICECPSGYTGKHCELNINECQSNPCRNQATCVDELNSYSC
KCQPGFSGKRCETGMYQLSVINNLNNAVCEDQVGGFLCKCPPGFLGTRCGKNVDECLSQPCKNGATCK
DGANSFRCLCAAGFTGSHCELNINECQSNPCRNQATCVDELNSYSCKCQPGFSGKRCETEQSTGFNLD
FEVSGIYGYVMLDGMLPSLHAITCTFWMKSSDDMNYGTPISYAVDNGSDNTLLLTDYNGWVLYVNGRE
KITNCPSVNDGRWHHIAITWTSTGGAWRVYINGELSDGGTGLSIGKAIPGGGALVLGQEQDKKGEGFN
PAESFVGSISQLNLWDYVLSPQQVKSLATSCPEELSKGNVLAWPDFLSGIVGKVKIDSKSIFCSDCPR
LGGSVPHLRTASEDLKPGSKVNLFCEPGFQLVGNPVQYCLNQGQWTQPLPHCERIRCGVPPPLENGFH
SADDFYAGSTVTYQCNNGYYLLGDSRMFCTDNGSWNGVSPSCLDVDECAVGSDCSEHASCLNVDGSYI
CSCVPPYTGDGKNCAEPIKCKAPGNPENGHSSGEIYTVGAEVTFSCQEGYQLMGVTKITCLESGEWNH
LIPYCKAVSCGKPAIPENGCIEELAFTFGSKVTYRCNKGYTLAGDKESSCLANSSWSHSPPVCEPVKC
SSPENINNGKYILSGLTYLSTASYSCDTGYSLQGPSIIECTASGIWDRAPPACHLVFCGEPPAIKDAV
ITGNNFTFRNTVTYTCKEGYTLAGLDTIECLADGKWSRSDQQCLAVSCDEPPIVDHASPETAHRLFGD
IAFYYCSDGYSLADNSQLLCNAQGKWVPPEGQDMPRCIAHFCEKPPSVSYSILESVSKAKFAAGSVVS
FKCMEGFVLNTSAKIECMRGGQWNPSPMSIQCIPVRCGEPPSIMNGYASGSNYSFGAMVAYSCNKGFY
IKGEKKSTCEATGQWSSPIPTCHPVSCGEPPKVENGFLEHTTGRIFESEVRYQCNPGYKSVGSPVFVC
QANRHWHSESPLMCVPLDCGKPPPIQNGFMKGENFEVGSKVQFFCNEGYELVGDSSWTCQKSGKWNKK
SNPKCMPAKCPEPPLLENQLVLKELTTEVGVVTFSCKEGHVLQGPSVLKCLPSQQWNDSFPVCKIVLC
TPPPLISFGVPIPSSALHFGSTVKYSCVGGFFLRGNSTTLCQPDGTWSSPLPECVPVECPQPEEIPNG
IIDVQGLAYLSTALYTCKPGFELVGNTTTLCGENGHWLGGKPTCKAIECLKPKEILNGKFSYTDLHYG
QTVTYSCNRGFRLEGPSALTCLETGDWDVDAPSCNAIHCDSPQPIENGFVEGADYSYGAIIIYSCFPG
FQVAGHAMQTCEESGWSSSIPTCMPIDCGLPPHIDFGDCTKLKDDQGYFEQEDDMMEVPYVTPHPPYH
LGAVAKTWENTKESPATHSSNFLYGTMVSYTCNPGYELLGNPVLICQEDGTWNGSAPSCISIECDLPT
APENGFLRFTETSMGSAVQYSCKPGHILAGSDLRLCLENRKWSGASPRCEAISCKKPNPVMNGSIKGS
NYTYLSTLYYECDPGYVLNGTERRTCQDDKNWDEDEPICIPVDCSSPPVSANGQVRGDEYTFQKEIEY
TCNEGFLLEGARSRVCLANGSWSGATPDCVPVRCATPPQLANGVTEGLDYGFMKEVTFHCHEGYILHG
APKLTCQSDGNWDAEIPLCKPVNCGPPEDLAHGFPNGFSFIHGGHIQYQCFPGYKLHGNSSRRCLSNG
SWSGSSPSCLPCRCSTPVIEYGTVNGTDFDCGKAARIQCFKGFKLLGLSEITCEADGQWSSGFPHCEH
TSCGSLPMIPNAFISETSSWKENVITYSCRSGYVIQGSSDLICTEKGVWSQPYPVCEPLSCGSPPSVA
NAVATGEAHTYESEVKLRCLEGYTMDTDTDTFTCQKDGRWFPERISCSPKKCPLPENITHILVHGDDF
SVNRQVSVSCAEGYTFEGVNISVCQLDGTWEPPFSDESCSPVSCGKPESPEHGFVVGSKYTFESTIIY
QCEPGYELEGNRERVCQENRQWSGGVAICKETRCETPLEFLNGKADIENRTTGPNVVYSCNRGYSLEG
PSEAHCTENGTWSHPVPLCKPNPCPVPFVIPENALLSEKEFYVDQNVSIKCREGFLLQGHGIITCNPD
ETWTQTSAKCEKISCGPPAHVENAIARGVHYQYGDMITYSCYSGYMLEGFLRSVCLENGTWTSPPICR
AVCRFPCQNGGICQRPNACSCPEGWMGRLCEEPICILPCLNGGRCVAPYQCDCPPGWTGSRCHTAVCQ
SPCLNGGKCVRPNRCHCLSSWTGHNCSRKRRTGF

| | | |
|---|---|---|
| NOV8k, SNP13380899 of | SEQ ID NO: 197 | 11158 bp |
| CG50646-04, | ORF Start: ATG at 77 | ORF Stop: TAA at 10787 |

| | | |
|---|---|---|
| DNA Sequence | SNP Pos: 2404 | SNP Change: A to G |

CAATTGGTCTAGGGTCTCCCCCATTGGAATATCCATCAGTGATGAGAAATACAACGTTTGTTGAGTTT
TCTCTAGCATGAGAAGAATTTGCGCGGCTTGCTGGGGTCTGGCGCTCGTTTCGGGCTGGGCGACCTTT
CAGCAGATGTCCCCGTCGCGCAATTTCAGCTTCCGCCTCTTCCCCGAGACCGCGCCCGGGGCCCCCGG
GAGTATCCCCGCGCCGCCCGCTCCTGGCGACGAAGCGGCGGGGAGCAGAGTGGAGCGGCTGGGCCAGG
CGTTCCGCGTGCGGCTGCTGCGGGAGCTCAGCGAGCGCCTGGAGCTTGTCTTCCTGGTGGATGATTCG
TCCAGCGTGGGCGAAGTCAACTTCCGCAGCGAGCTCATGTTCGTCCGCAAGCTGCTGTCCGACTTCCC
CGTGGTGCCCACGGCCACGCGCGTGGCCATCGTGACCTTCTCGTCCAAGAACTACGTGGTGCCGCGCG
TCGATTACATCTCCACCCGCCGCGCGCGCCAGCACAAGTGCGCGCTGCTCCTCCAAGAGATCCCTGCC
ATCTCCTACCGAGGTGGCGGCACCTACACCAAGGGCGCCTTCCAGCAAGCCGCGCAAATTCTTCTTCA
TGCTAGAGAAAACTCAACAAAAGTTGTATTTCTCATCACTGATGGATATTCCAATGGGGGAGACCCTA
GACCAATTGCAGCGTCACTGCGAGATTCAGGAGTGGAGATCTTCACTTTTGGCATATGGCAAGGGAAC
ATTCGAGAGCTGAATGACATGGCTTCCACCCCAAAGGAGGAGCACTGTTACCTGCTACACAGTTTTGA
AGAATTTGAGGCTTTAGTCGCCCTCTGTCATATGTTATTTGTAGATCTACCTTCTGGGAGTTTTATTC
AAGATGATATGGTCCACTGCTCATATCTTTGTGATGAAGGCAAGGACTGCTGTGACCGAATGGGAAGC
TGCAAATGTGGGAAACACACAGGCCATTTTGAGTGCATCTGTGAAAAGGGGTATAACGGGAAAGGTCT
GCAGTATGACTGCACAGTTTGCCCATCGGGGACATACAAACCTGAAGGCTCACCAGGAGGAATCAGCA
GTTGCATTCCATGTCCTGATGAAAATCACACCTCTCCACCTGGAAGCACATCCCCTGAAGACTGTGTC
TGCAGAGAGGGATACAGGGCATCTGGCCAGACCTGTGAAGTTGTCCACTGCCCTGCCCTGAAGCCTCC
CGAAAATGGTTACTTTATCCAAAACACTTGCAACAACCACTTCAATGCAGCCTGTGGGGTCCGATGTC
ACCCTGGATTTGATCTTGTGGGAAGCAGCATCATCTTATGTCTACCCAATGGTTTGTGGTCCGGTTCA
GAGAGCTACTGCAGAGTAAGAACATGTCCTCATCTCCGCCAGCCGAAACATGGCCACATCAGCTGTTC
TACAAGGGAAATGTTATATAAGACAACATGTTTGGTTGCCTGTGATGAAGGGTACAGGCTAGAAGGCA
GTGATAAGCTTACTTGTCAAGGAAACAGCCAGTGGGATGGGCCAGAACCCCGGTGTGTGGAGCGCCAC
TGTTCCACCTTTCAGATGCCCAAAGATGTCATCATATCCCCCCACAACTGTGGCAAGCAGCCAGCCAA
ATTTGGGACGATCTGCTATGTAAGTTGCCGCCAAGGGTTCATTTTATCTGGAGTCAAAGAAATGCTGA
GATGTACCACTTCTGGAAAATGGAATGTCGGAGTTCAGGCAGCTGTGTGTAAAGACGTGGAGGCTCCT
CAAATCAACTGTCCTAAGGACATAGAGGCTAAGACTCTGGAACAGCAAGATTCTGCCAATGTTACCTG
GCAGATTCCAACAGCTAAAGACAACTCTGGTGAAAAGGTGTCAGTCCACGTTCATCCAGCTTTCACCC
CACCTTACCTTTTCCCAATTGGAGATGTTGCTATCGTATACACGGCAACTGACCTATCCGGCAACCAG
GCCAGCTGCATTTTCCATATCAAGGTTATTGATGCAGAACCACCTGTCATAGACTGGTGCAGATCTCC
ACCTCCCGTCCAGGTCTCGGAGAAGGTACATGCCGCAAGCTGGGATGAGCCTCAGTTCTCAGACAACT
CAGGTGCTGAATTGGTCATTACCAGAAGTCATACACAAGGAGACCTTTTCCCTCAAGGGGAGACTATA
GTACAGTATACAGCCACTGACCCCTCAGGCAATAACAGGACATGTGATATCCATATTGTCATAAAAGG
TTCTCCCTGTGAAATTCCATTCACACCTGTAAATGGGGATTTTATATGCACTCCAGATAATACTGGAG
TCAACTGTACATTAACTTGCTTGGAGGGCTATGATTTCACAGAAGGGTCTACTGACAAGTATTATTGT
GCTTATGAAGATGGCGTCTGGAAGCCAACATATACCACTGAATGGCCAGACTGTGCCAGTAAGCGTTT
TGCAAACCACGGGTTCAAGTCCTTTGAGATGTTCTACAAAGCAGCTCGTTGTGATGACACAGATCTGA
TGAAGAAGTTTTCTGAAGCATTTGAGACGACCCTGGGAAAAATGGTCCCATCATTTTGTAGTGATGCA
GAGGACATTGACTGCAGACTGGAGGAGAACCTGACCAAAAAATATTGCCTAGAATATAATTATGACTA
TGAAAATGGCTTTGCAATTGGTCCAGGTGGCTGGGGTGCAGCTCATAGGCTGGATTACTCTTACGATG
ACTTCCTGGACACTGTGCAAGAAACAGCCACAAGCATCGGCAATGCCAAGTCCTCACGGATTAAAAGA
AGTGCCCCATTATCTGACTATAAAATTAAGTTAATTTTTAACATCACAGCTAGTGTGCCATTACCCGA
TGAAAGAAATGATACCCTTGAATGGGAAAATCAGCAACGACTCCTTCAGACATTGGAAACTATCACAA
ATAAACTGAAAAGGACTCTCAACAAAGACCCCATGTATTCCTTTCAGCTTGCATCAGAAATACTTATA
GCCGACAGCAATTCATTAGAAACAAAAAAGGCTTCCCCCTTCTGCAGACCAGGCTCAGTGCTGAGAGG
GCGTATGTGTGTCAATTGCCCTTTGGGAACCTATTATAATCTGGAACATTTCACCTGTAGAAACTGCC
GGATCGGATCCTATCAAGATGAAGAAGGGCAACTTGAGTGCAAGCTTTGCCCCTCTGGGATGTACACG
GAATATATCCATTCAAGAAACATCTCTGATTGTAAAGCTCAGTGTAAACAAGGCACCTACTCATACAG
TGGACTTGAGACTTGTGAATCGTGTCCACTGGGCACTTATCAGCCAAAATTTGGTTCCCGGAGCTGCC
TCTCGTGTCCAGAAAACACCTCAACTGTGAAAAGAGGAGCCGTGAACATTTCTGCATGTGGAGTTCCT
GATCCATCACAGAATGTTCAAGTTTTAGTTCAACTTTCTCAGCGGCAGAGGAAAGTGTGGTGCCCCCT
CCCTTGCCACAATAGTGGAACCTGCCAGCAACTTGGGCGTGGTTATGTTTGTCTCTGTCCACTTGGAT
ATACAGGTTTAAAGTGTGAAACAGACATCGATGAGTGCAGCCCACTGCCTTGCCTCAACAATGGAGTT
TGTAAAGACCTAGTTGGGGAATTCATTTGTGAGTGCCCATCAGGTTACACAGGTAAGCACTGTGAATT
GAACATCAATGAATGTCAGTCTAATCCATGTAGAAATCAGGCCACCTGTGTGGATGAATTAAATTCAT
ACAGTTGTAAATGTCAGCCAGGATTTTCAGGCAAGGTGTGAAACAGGTATGTATCGTATCTCAGTGTT
ATTAATAACCTTAATAATGCAGTCTGTGAAGACCAGGTTGGGGGATTCTTGTGCAAATGCCCACCTGG
ATTTTTGGGTACCCGATGTGGAAAGAACGTCGATGAGTGTCTCAGTCAGCCATGCAAAAATGGAGCTA
CCTGTAAAGACGGTGCCAATAGCTTCAGGTGCCTGTGTGCAGCTGGCTTCACAGGATCACACTGTGAA
TTGAACATCAATGAATGTCAGTCTAATCCATGTAGAAATCAGGCCACCTGTGTGGATGAATTAAATTC
ATACAGTTGTAAATGTCAGCCAGGATTTTCAGGCAAAAGGTGTGAAACAGAACAGTCTACAGGCTTTA
ACCTGGATTTTGAAGTTTCTGGCATCTATGGATATGTCATGCTAGATGGCATGCTCCCATCTCTCCAT
GCTCTAACCTGTACCTTCTGGATGAAATCCTCTGACGACATGAACTATGGAACACCAATCTCCTATGC
AGTTGATAACGGCAGCGACAATACCTTGCTCCTGACTGATTATAACGGGTGGGTTCTTTATGTGAATG
GCAGGGAAAAGATAACAAACTGTCCCTCGGTGAATGATGGCAGATGGCATCATATTGCAATCACTTGG
ACAAGTACTGGTGGAGCCTGGAGGGTCTATATAAATGGGGAATTATCTGACGGTGGTACTGGCCTCTC
CATTGGCAAAGCCATACCTGGTGGCGGTGCATTAGTTCTTGGGCAAGAGCAAGACAAAAAAGGAGAGG
GGTTCAACCCGGCTGAGTCTTTTGTGGGCTCCATAAGCCAGCTCAACCTCTGGGACTATGTCCTGTCT
CCACAGCAGGTGAAGTCACTGGCTACCTCCTGCCCAGAGGAACTCAGTAAAGGAAACGTGTTAGCATG
GCCTGATTTCTTGTCAGGAATTGTGGGGAAAGTGAAGATCGATTCTAAGAGCATATTTTGTTCTGATT
GCCCACGCTTGGGAGGGTCAGTGCCTCATCTGAGAACTGCATCTGAAGATTTAAAACCAGGTTCCAAA
GTCAATCTGTTCTGTGAACCAGGCTTCCAGCTGGTCGGGAACCCTGTGCAGTACTGTCTGAATCAAGG
ACAGTGGACACAACCACTCCCCCACTGTGAACGCATTCGCTGTGGGGTGCCACCTCCTTTGGAGAATG
GCTTCCATTCAGCCGATGACTTCTATGCTGGCAGCACAGTAACCTACCAGTGCAACAATGGCTACTAT
CTATTGGGTGACTCAAGGATGTTCTGTACAGATAATGGGAGCTGGAACGGCGTTTCACCATCCTGCTT
AGATGTCGATGAGTGTGCAGTTGGATCAGATTGTAGTGAGCATGCTTCTTGCCTGAACGTAGATGGAT
CCTACATATGTTCATGTGTCCCACCGTACACAGGAGATGGGAAAAACTGTGCAGAACCTATAAAATGT
AAGGCTCCAGGAAATCCGGAAAATGGCCACTCCTCAGGTGAGATTTATACAGTAGGTGCCGAAGTCAC
ATTTTCGTGTCAGGAAGGATACCAGTTGATGGGAGTAACCAAAATCACATGTTTGGAGTCTGGAGAAT
GGAATCATCTAATACCATATTGTAAAGCTGTTTCATGTGGTAAACCGGCTATTCCAGAAAATGGTTGC
ATTGAGGAGTTAGCATTTACTTTTGGCAGCAAAGTGACATATAGGTGTAATAAAGGATATACTCTGGC
CGGTGATAAAGAATCATCCTGTCTTGCTAACAGTTCTTGGAGTCATTCCCCTCCTGTGTGTGAACCAG
TGAAGTGTTCTAGTCCGGAAAATATAAATAATGGAAAATATATTTTGAGTGGGCTTACCTACCTTTCT
ACTGCATCATATTCATGCGATACAGGATACAGCTTACAGGGCCCTTCCATTATTGAATGCACGGCTTC
TGGCATCTGGGACAGAGCGCCACCTGCCTGTCACCTCGTCTTCTGTGGAGAACCACCTGCCATCAAAG
ATGCTGTCATTACGGGGAATAACTTCACTTTCAGGAACACCGTCACTTACACTTGCAAAGAAGGCTAT
ACTCTTGCTGGTCTTGACACCATTGAATGCCTGGCCGACGGCAAGTGGAGTAGAAGTGACCAGCAGTG
CCTGGCTGTCTCCTGTGATGAGCCACCCATTGTGGACCACGCCTCTCCAGAGACTGCCCATCGGCTCT
TTGGAGACATTGCATTCTACTACTGCTCTGATGGTTACAGCCTAGCAGACAATTCCCAGCTTCTCTGC
AATGCCCAGGGCAAGTGGGTACCCCCAGAAGGTCAAGACATGCCCCGTTGTATAGCTCATTTCTGTGA
AAAACCTCCATCGGTTTCCTATAGCATCTTGGAATCTGTGAGCAAAGCAAAATTTGCAGCTGGCTCAG
TTGTGAGCTTTAAATGCATGGAAGGCTTTGTACTGAACACCTCAGCAAAGATTGAATGTATGAGAGGT
GGGCAGTGGAACCCTTCCCCCATGTCCATCCAGTGCATCCCTGTGCGGTGTGGAGAGCCACCAAGCAT
CATGAATGGCTATGCAAGTGGATCAAACTACAGTTTTGGAGCCATGGTGGCTTACAGCTGCAACAAGG
GGTTCTACATCAAAGGGGAAAAGAAGAGCACCTGCGAAGCCACAGGGCAGTGGAGTAGTCCTATACCG
ACGTGCCACCCGGTATCTTGTGGTGAACCACCTAAGGTTGAGAATGGCTTTCTGGAGCATACAACTGG
CAGGATCTTTGAGAGTGAAGTGAGGTATCAGTGTAACCCGGGCTATAAGTCAGTCGGAAGTCCTGTAT
TTGTCTGCCAAGCCAATCGCCACTGGCACAGTGAATCCCCTCTGATGTGTGTTCCTCTCGACTGTGGA
AAACCTCCCCCGATCCAGAATGGCTTCATGAAAGGAGAAAACTTTGAAGTAGGGTCCAAGGTTCAGTT
TTTCTGTAATGAGGGTTATGAGCTTGTTGGTGACAGTTCTTGGACATGTCAGAAATCTGGCAAATGGA
ATAAGAAGTCAAATCCAAAGTGCATGCCTGCCAAGTGCCCAGAGCCGCCCCTCTTGGAAAACCAGCTA
GTATTAAAGGAGTTGACCACCGAGGTAGGAGTTGTGACATTTTCCTGTAAAGAAGGGCATGTCCTGCA
AGGCCCCTCTGTCCTGAAATGCTTGCCATCCCAGCAATGGAATGACTCTTTCCCTGTTTGTAAGATTG
TTCTTTGTACCCCACCTCCCCTAATTTCCTTTGGTGTCCCCATTCCTTCTTCTGCTCTTCATTTTGGA
AGTACTGTCAAGTATTCTTGTGTAGGTGGGTTTTTCCTAAGAGGAAATTCTACCACCCTCTGCCAACC
TGATGGCACCTGGAGCTCTCCACTGCCAGAATGTGTTCCAGTAGAATGTCCCCAACCTGAGGAAATCC
CCAATGGAATCATTGATGTGCAAGGCCTTGCCTATCTCAGCACAGCTCTCTATACCTGCAAGCCAGGC
TTTGAATTGGTGGGAAATACTACCACCCTTTGTGGAGAAAATGGTCACTGGCTTGGAGGAAAACCAAC
ATGTAAAGCCATTGAGTGCCTGAAACCCAAGGAGATTTTGAATGGCAAATTCTCTTACACGGACCTAC
ACTATGGACAGACCGTTACCTACTCTTGCAACCGAGGCTTTCGGCTCGAAGGTCCCAGTGCCTTGACC
TGTTTAGAGACAGGTGATTGGGATGTAGATGCCCCATCTTGCAATGCCATCCACTGTGATTCCCCACA
ACCCATTGAAAATGGTTTTGTAGAAGGTGCAGATTACAGCTATGGTGCCATAATCATCTACAGTTGCT
TCCCTGGGTTTCAGGTGGCTGGTCATGCCATGCAGACCTGTGAAGAGTCAGGATGGTCAAGTTCCATC
CCAACATGTATGCCAATAGACTGTGGCCTCCCTCCTCATATAGATTTTGGAGACTGTACTAAACTCAA
AGATGACCAGGGATATTTTGAGCAAGAAGACGACATGATGGAAGTTCCATATGTGACTCCTCACCCTC
CTTATCATTTGGGAGCAGTGGCTAAAACCTGGGAAAATACAAAGGAGTCTCCTGCTACACATTCATCA
AACTTTCTGTATGGTACCATGGTTTCATACACCTGTAATCCAGGATATGAACTTCTGGGGAACCCTGT
GCTGATCTGCCAGGAAGATGGAACTTGGAATGGCAGTGCACCATCCTGCATTTCAATTGAATGTGACT
TGCCTACTGCTCCTGAAAATGGCTTTTTGCGTTTTACAGAGACTAGCATGGGAAGTGCTGTGCAGTAT
AGCTGTAAACCTGGACACATTCTAGCAGGCTCTGACTTAAGGCTTTGTCTAGAGAATAGAAAGTGGAG
TGGTGCCTCCCCACGCTGTGAAGCCATTTCATGCAAAAAGCCAAATCCAGTCATGAATGGATCCATCA
AAGGAAGCAACTACACATACCTGAGCACGTTGTACTATGAGTGTGACCCCGGATATGTGCTGAATGGC
ACTGAGAGGAGAACATGCCAGGATGACAAAAACTGGGATGAGGATGAGCCCATTTGCATTCCTGTGGA
CTGCAGTTCACCCCCAGTCTCAGCCAATGGCCAGGTGAGAGGAGACGAGTACACATTCCAAAAAGAGA
TTGAATACACTTGCAATGAAGGGTTCTTGCTTGAGGGAGCCAGGAGTCGGGTTTGTCTTGCCAATGGA
GGTGACGGAAGGCCTGGACTATGGCTTCATGAAGGAAGTAACATTCCACTGTCACGAGGGCTACATCT
TGCACGGTGCTCCAAAACTCACCTGTCAGTCAGATGGCAACTGGGATGCAGAGATTCCTCTCTGTAAA
CCAGTCAACTGTGGACCTCCTGAAGATCTTGCCCATGGTTTCCCTAATGGTTTTTCCTTTATTCATGG
GGGCCATATACAGTATCAGTGCTTTCCTGGTTATAAGCTCCATGGAAATTCATCAAGAAGGTGCCTCT
CCAATGGCTCCTGGAGTGGCAGCTCACCTTCCTGCCTGCCTTGCAGATGTTCCACACCAGTAATTGAA
TATGGAACTGTCAATGGGACAGATTTTGACTGTGGAAAGGCAGCCCGGATTCAGTGCTTCAAAGGCTT
CAAGCTCCTAGGACTTTCTGAAATCACCTGTGAAGCCGATGGCCAGTGGAGCTCTGGGTTCCCCCACT
AAGGAAAATGTGATAACTTACAGCTGCAGGTCTGGATATGTCATACAAGGCAGTTCAGATCTGATTTG
TACAGAGAAAGGGGTATGGAGCCAGCCTTATCCAGTCTGTGAGCCCTTGTCCTGTGGGTCCCCACCGT
CTGTCGCCAATGCAGTGGCAACTGGAGAGGCACACACCTATGAAAGTGAAGTGAAACTCAGATGTCTG
GAAGGTTATACGATGGATACAGATACAGATACATTCACCTGTCAGAAAGATGGTCGCTGGTTCCCTGA
GAGAATCTCCTGCAGTCCTAAAAAATGTCCTCTCCCGGAAAACATAACACATATACTTGTACATGGGG
ACGATTTCAGTGTGAATAGGCAAGTTTCTGTGTCATGTGCAGAAGGGTATACCTTTGAGGGAGTTAAC
ATATCAGTATGTCAGCTTGATGGAACCTGGGAGCCACCATTCTCCGATGAATCTTGCAGTCCAGTTTC
TTGTGGGAAACCTGAAAGTCCAGAACATGGATTTGTGGTTGGCAGTAAATACACCTTTGAAAGCACAA
TTATTTATCAGTGTGAGCCTGGCTATGAACTAGAGGGGAACAGGGAACGTGTCTGCCAGGAGAACAGA
CAGTGGAGTGGAGGGGTGGCAATATGCAAAGAGACCAGGTGTGAAACTCCACTTGAATTTCTCAATGG
GAAAGCTGACATTGAIAACAGGACGACTGGACCCAACGTGGTATATTCCTGCAACAGAGGCTACAGTC
TTGAAGGGCCATCTGAGGCACACTGCACAGAAAATGGAACCTGGAGCCACCCAGTCCCTCTCTGCAAA
CCAAATCCATGCCCTGTTCCTTTTGTGATTCCCGAGAATGCTCTGCTGTCTGAAAAGGAGTTTTATGT
TGATCAGAATGTGTCCATCAAATGTAGGGAAGGTTTTCTGCTGCAGGGCCACGGCATCATTACCTGCA
ACCCCGACGAGACGTGGACACAGACAAGCGCCAAATGTGAAAAAATCTCATGTGGTCCACCAGCTCAC
GTAGAAAATGCAATTGCTCGAGGCGTACATTATCAATATGGAGACATGATCACCTACTCATGTTACAG
TGGATACATGTTGGAGGGTTTCCTGAGGAGTGTTTGTTTAGAAAATGGAACATGGACATCACCTCCTA
TTTGCAGAGCTGTCTGTCGATTTCCATGTCAGAATGGGGGCATCTGCCAACGCCCAAATGCTTGTTCC
TGTCCAGAGGGCTGGATGGGGCGCCTCTGTGAAGAACCAATCTGCATTCTTCCCTGTCTGAACGGAGG
TCGCTGTGTGGCCCCTTACCAGTGTGACTGCCCGCCTGGCTGGACGGGGTCTCGCTGTCATACAGCTG
TTTGCCAGTCTCCCTGCTTAAATGGTGGAAAATGTGTAAGACCAAACCGATGTCACTGTCTTTCTTCT
TGGACGGGACATAACTGTTCAAGGAAAAGGAGGACTGGGTTTTAACCACTGCACGACCATCTGGCTCT
CCCAAAAGCAGGATCATCTCTCCTCGGTAGTGCCTGGGCATCCTGGAACTTATGCAAAGAAAGTCCAA
CATGGTGCTGGGTCTTGTTTAGTAAACTTGTTACTTGGGGTTACTTTTTTTATTTTGTGATATATTTT
GTTATTCCTTGTGACATACTTTCTTACATGTTTCCATTTTTAAATATGCCTGTATTTTCTATATAAAA
ATTATATTAAATAGATGCTGCTCTACCCTCACAAAATGTACATATTCTGCTGTCTATTGGGAAAGTTC
CTGGTACACATTTTTATTCAGTTACTTAAAATGATTTTTCCATTAAAGTATATTTTGCTACTAAATAA
AAAAAA

| | | |
|---|---|---|
| NOV8k, SNP13380899 of | SEQ ID NO: 198 | MW at 390012.5kD |

| | | | |
|---|---|---|---|
| CG50646-04, Protein Sequence | SNP Pos: 776 | 3570 aa | SNP Change: Lys to Lys |

MRRICAACWGLALVSGWATFQQMSPSRNFSFRLFPETAPGAPGSIPAPPAPGDEAAGSRVERLGQAFR
VRLLRELSERLELVFLVDDSSSVGEVNFRSELMFVRKLLSDFPVVPTATRVAIVTFSSKNYVVPRVDY
ISTRRARQHKCALLLQEIPAISYRGGGTYTKGAFQQAAQILLHARENSTKVVFLITDGYSNGGDPRPI
AASLRDSGVEIFTFGIWQGNIRELNDMASTPKEEHCYLLHSFEEFEAIVALCHMLFVDLPSGSFIQDD
MVHCSYLCDEGKDCCDRMGSCKCGKHTGHFECICEKGYNGKGLQYDCTVCPSGTYKPEGSPGGISSCI
PCPDENHTSPPGSTSPEDCVCREGYRASGQTCEVVHCPALKPPENGYFIQNTCNNHFNAACGVRCHPG
FDLVGSSIILCLPNGLWSGSESYCRVRTCPHLRQPKHGHISCSTREMLYKTTCLVACDEGYRLEGSDK
LTCQGNSQWDGPEPRCVERHCSTFQMPKDVIISPHNCGKQPAKFGTICYVSCRQGFILSGVKEMLRCT
TSGKWNVGVQAAVCKDVEAPQINCPKDIEAKTLEQQDSANVTWQIPTAKDNSGEKVSVHVHPAFTPPY
LFPIGDVAIVYTATDLSGNQASCIFHIKVIDAEPPVIDWCRSPPPVQVSEKVHAASWDEPQFSDNSGA
ELVITRSHTQGDLFPQGETIVQYTATDPSGNNRTCDIHIVIKGSPCEIPFTPVNGDFICTPDNTGVNC
TLTCLEGYDFTEGSTDKYYCAYEDGVWKPTYTTEWPDCASKRFANHGFKSFEMFYKAARCDDTDLMKK
FSEAFETTLGKMVPSFCSDAEDIDCRLEENLTKYYCLEYNYDYENGFAIGPGGWGAANRLDYSYDDFL
DTVQETATSIGNAKSSRIKRSAPLSDYKIKLIFNITASVPLPDERNDTLEWENQQRLLQTLETITNKL
KRTLNKDPMYSFQLASEILIADSNSLETKKASPFCRPGSVLRGRMCVNCPLGTYYNLEHFTCESCRIG
SYQDEEGQLECKLCPSGMYTEYIHSRNISDCKAQCKQGTYSYSGLETCESCPLGTYQPKFGSRSCLSC
PENTSTVKRGAVNISACGVPCPEGKFSRSGLMPCHPCPRDYYQPNAGKAFCLACPFYGTTPFAGSRSI
TECSSFSSTFSAAEESVVPPASLGHIKKRHEISSQASHECFFNPCHNSGTCQQLGRGYVCLCPLGYTG
LKCETDIDECSPLPCLNNGVCKDLVGEFICECPSGYTGKHCELNINECQSNPCRNQATCVDELNSYSC
KCQPGFSGKRCETGMYQLSVINNLNNAVCEDQVGGFLCKCPPGFLGTRCGKNVDECLSQPCKNGATCK
DGANSFRCLCAAGFTGSHCELNINECQSNPCRNQATCVDELNSYSCKCQPGFSGKRCETEQSTGFNLD
FEVSGIYGYVMLDGMLPSLHALTCTFWMKSSDDMNYGTPISYAVDNGSDNTLLLTDYNGWVLYVNGRE
KITNCPSVNDGRWHHIAITWTSTGGAWRVYINGELSDGGTGLSIGKAIPGGGALVLGQEQDKKGEGFN
PAESFVGSISQLNLWDYLSPQQVKSLATSCPEELSKGNVLAWPDFLSGIVGKVKIDSKSIFCSDCPR
LGGSVPHLRTASEDLKPGSKVNLFCEPGFQLVGNPVQYCLNQGQWTQPLPHCERIRCGVPPPLENGFH
SADDFYAGSTVTYQCNNGYYLLGDSRMFCTDNGSWNGVSPSCLDVDECAVGSDCSEHASCLNVDGSYI
CSCVPPYTGDGKNCAEPIKCKAPGNPENGHSSGEIYTVGAEVTFSCQEGYQLMGVTKITCLESGEWNH
LIPYCKAVSCGKPAIPENGCIEELAFTFGSKVTYRCNKGYTLAGDKESSCLANSSWSHSPPVCEPVKC
SSPENINNGKYILSGLTYLSTASYSCDTGYSLQGPSIIECTASGIWDRAPPACHLVFCGEPPAIKDAV
ITGNNFTFRNTVTYTCKEGYTLAGLDTIECLADGKWSRSDQQCLAVSCDEPPIVDHASPETAHRLFGD
IAFYYCSDGYSLADNSQLLCNAQGKWVPPEGQDMPRCIAHFCEKPPSVSYSILESVSKAKFAAGSVVS
FKCMEGFVLNTSAKIECMRGGQWNPSPMSIQCIPVRCGEPPSIMNGYASGSNYSFGAMVAYSCNKGFY
IKGEKKSTCEATGQWSSPIPTCHPVSCGEPPKVENGFLEHTTGRIFESEVRYQCNPGYKSVGSPVFVC
QANRHWHSESPLMCVPLDCGKPPPIQNGFMKGENFEVGSKVQFFCNEGYELVGDSSWTCQKSGKWNKK
SNPKCMPAKCPEPPLLENQLVLKELTTEVGVVTFSCKEGHVLQGPSVLKCLPSQQWNDSFPVCKIVLC
TPPPLISFGVPIPSSALHFGSTVKYSCVGGFFLRGNSTTLCQPDGTWSSPLPECVPVECPQPEEIPNG
IIDVQGLAYLSTALYTCKPGFELVGNTTTLCGENGHWLGGKPTCKAIECLKPKEILNGKFSYTDLHYG
QTVTYSCNRGFRLEGPSALTCLETGDWDVDAPSCNAIHCDSPQPIENGFVEGADYSYGAIIIYSCFPG
FQVAGHAMQTCEESGWSSSIPTCMPIDCGLPPHIDFGDCTKLKDDQGYFEQEDDMNEVPYVTPHPPYH
LGAVAKTWENTKESPATHSSNFLYGTMVSYTCNPGYELLGNPVLICQEDGTWNGSAPSCISIECDLPT
APENGFLRFTETSMGSAVQYSCKPGHILAGSDLRLCLENRKWSGASPRCEAISCKKPNPVMNGSIKGS
NYTYLSTLYYECDPGYVLNGTERRTCQDDKNWDEDEPICIPVDCSSPPVSANGQVRGDEYTFQKEIEY
TCNEGFLLEGARSRVCLANGSWSGATPDCVPVRCATPPQLANGVTEGLDYGFMKEVTFHCHEGYILHG
APKLTCQSDGNWDAEIPLCKPVNCGPPEDLAHGFPNGFSFIHGGHIQYQCFPGYKLHGNSSRRCLSNG
SWSGSSPSCLPCRCSTPVIEYGTVNGTDFDCGKAARIQCFKGFKLLGLSEITCEADGQWSSGFPHCEH
TSCGSLPMIPNAFISETSSWKENVITYSCRSGYVIQGSSDLICTEKGVWSQPYPVCEPLSCGSPPSVA
NAVATGEAHTYESEVKLRCLEGYTMDTDTDTFTCQKDGRWFPERISCSPKKCPLPENITHILVHGDDF
SVNRQVSVSCAEGYTFEGVNISVCQLDGTWEPPFSDESCSPVSCGKPESPEHGFVVGSKYTFESTIIY
QCEPGYELEGNRERVCQENRQWSGGVAICKETRCETPLEFLNGKADIENRTTGPNVVYSCNRGYSLEG
PSEAHCTENGTWSHPVPLCKPNPCPVPFVIPENALLSEKEFYVDQNVSIKCREGFLLQGHGIITCNPD
ETWTQTSAKCEKISCGPPAHVENAIARGVHYQYGDMITYSCYSGYMLEGFLRSVCLENGTWTSPPICR
AVCRFPCQNGGICQRPNACSCPEGWMGRLCEEPICILPCLNGGRCVAPYQCDCPPGWTGSRCHTAVCQ
SPCLNGGKCVRPNRCHCLSSWTGHNCSRKRRTGF

| | |
|---|---|
| SEQ ID NO: 199 | 11158 bp |

| | | |
|---|---|---|
| NOV8l, SNP13374702 of | ORF Start: ATG at 77 | ORF Stop: TAA at 10787 |

| | | |
|---|---|---|
| CG50646-04, DNA Sequence | SNP Pos: 2457 | SNP Change: A to G |

CAATTGGTCTAGGGTCTCCCCCATTGGAATATCCATCAGTGATGAGAAATACAACGTTTGTTGAGTTT
TCTCTAGCATGAGAAGAATTTGCGCGGCTTGCTGGGGTCTGGCGCTCGTTTCGGGCTGGGCGACCTTT
CAGCAGATGTCCCCGTCGCGCAATTTCAGCTTCCGCCTCTTCCCCGAGACCGCGCCCGGGGCCCCCGG
GAGTATCCCCGCGCCGCCCGCTCCTGGCGACGAAGCGGCGGGGAGCAGAGTGGAGCGGCTGGGCCAGG
CGTTCCGCGTGCGGCTGCTGCGGGAGCTCAGCGAGCGCCTGGAGCTTGTCTTCCTGGTGGATGATTCG
TCCAGCGTGGGCGAAGTCAACTTCCGCAGCGAGCTCATGTTCGTCCGCAAGCTGCTGTCCGACTTCCC
CGTGGTGCCCACGGCCACGCGCGTGGCCATCGTGACCTTCTCGTCCAAGAACTACGTGGTGCCGCGCG
TCGATTACATCTCCACCCGCCGCGCGCGCCAGCACAAGTGCGCGCTGCTCCTCCAAGAGATCCCTGCC
ATCTCCTACCGAGGTGGCGGCACCTACACCAAGGGCGCCTTCCAGCAAGCCGCGCAAATTCTTCTTCA
TGCTAGAGAAAACTCAACAAAAGTTGTATTTCTCATCACTGATGGATATTCCAATGGGGGAGACCCTA
GACCAATTGCAGCGTCACTGCGAGATTCAGGAGTGGAGATCTTCACTTTTGGCATATGGCAAGGGAAC
ATTCGAGAGCTGAATGACATGGCTTCCACCCCAAAGGAGGAGCACTGTTACCTGCTACACAGTTTTGA
AGAATTTGAGGCTTTAGTCGCCCTCTGTCATATGTTATTTGTAGATCTACCTTCTGGGAGTTTTATTC
AAGATGATATGGTCCACTGCTCATATCTTTGTGATGAAGGCAAGGACTGCTGTGACCGAATGGGAAGC
TGCAAATGTGGGAAACACACAGGCCATTTTGAGTGCATCTGTGAAAAGGGGTATAACGGGAAAGGTCT
GCAGTATGACTGCACAGTTTGCCCATCGGGGACATACAAACCTGAAGGCTCACCAGGAGGAATCAGCA
GTTGCATTCCATGTCCTGATGAAAATCACACCTCTCCACCTGGAAGCACATCCCCTGAAGACTGTGTC
TGCAGAGAGGGATACAGGGCATCTGGCCAGACCTGTGAAGTTGTCCACTGCCCTGCCCTGAAGCCTCC
CGAAAATGGTTACTTTATCCAAAACACTTGCAACAACCACTTCAATGCAGCCTGTGGGGTCCGATGTC
ACCCTGGATTTGATCTTGTGGGAAGCAGCATCATCTTATGTCTACCCAATGGTTTGTGGTCCGGTTCA
GAGAGCTACTGCAGAGTAAGAACATGTCCTCATCTCCGCCAGCCGAAACATGGCCACATCAGCTGTTC
TACAAGGGAAATGTTATATAAGACAACATGTTTGGTTGCCTGTGATGAAGGGTACAGGCTAGAAGGCA
GTGATAAGCTTACTTGTCAAGGAAACAGCCAGTGGGATGGGCCAGAACCCCGGTGTGTGGAGCGCCAC
TGTTCCACCTTTCAGATGCCCAAAGATGTCATCATATCCCCCCACAACTGTGGCAAGCAGCCAGCCAA
ATTTGGGACGATCTGCTATGTAAGTTGCCGCCAAGGGTTCATTTTATCTGGAGTCAAAGAAATGCTGA
GATGTACCACTTCTGGAAAATGGAATGTCGGAGTTCAGGCAGCTGTGTGTAAAGACGTGGAGGCTCCT
CAAATCAACTGTCCTAAGGACATAGAGGCTAAGACTCTGGAACAGCAAGATTCTGCCAATGTTACCTG
GCAGATTCCAACAGCTAAAGACAACTCTGGTGAAAAGGTGTCAGTCCACGTTCATCCAGCTTTCACCC
CACCTTACCTTTTCCCAATTGGAGATGTTGCTATCGTATACACGGCAACTGACCTATCCGGCAACCAG
GCCAGCTGCATTTTCCATATCAAGGTTATTGATGCAGAACCACCTGTCATAGACTGGTGCAGATCTCC
ACCTCCCGTCCAGGTCTCGGAGAAGGTACATGCCGCAAGCTGGGATGAGCCTCAGTTCTCAGACAACT
CAGGTGCTGAATTGGTCATTACCAGAAGTCATACACAAGGAGACCTTTTCCCTCAAGGGGAGACTATA
GTACAGTATACAGCCACTGACCCCTCAGGCAATAACAGGACATGTGATATCCATATTGTCATAAAAGG
TTCTCCCTGTGAAATTCCATTCACACCTGTAAATGGGGATTTTATATGCACTCCAGATAATACTGGAG
TCAACTGTACATTAACTTGCTTGGAGGGCTATGATTTCACAGAAGGGTCTACTGACAAGTATTATTGT
GCTTATGAAGATGGCGTCTGGAAACCAACATATACCACTGAATGGCCAGACTGTGCCAGTAAGCGTTT
TGCAAACCGCGGGTTCAAGTCCTTTGAGATGTTCTACAAAGCAGCTCGTTGTGATGACACAGATCTGA
TGAAGAAGTTTTCTGAAGCATTTGAGACGACCCTGGGAAAAATGGTCCCATCATTTTGTAGTGATGCA
GAGGACATTGACTGCAGACTGGAGGAGAACCTGACCAAAAAATATTGCCTAGAATATAATTATGACTA
TGAAAATGGCTTTGCAATTGGTCCAGGTGGCTGGGGTGCAGCTAATAGGCTGGATTACTCTTACGATG
ACTTCCTGGACACTGTGCAAGAAACAGCCACAAGCATCGGCAATGCCAAGTCCTCACGGATTAAAAGA
AGTGCCCCATTATCTGACTATAAAATTAAGTTAATTTTTAACATCACAGCTAGTGTGCCATTACCCGA
ATAAACTGAAAAGGACTCTCAACAAAGACCCCATGTATTCCTTTCAGCTTGCATCAGAAATACTTATA
GCCGACAGCAATTCATTAGAAACAAAAAAGGCTTCCCCCTTCTGCAGACCAGGCTCAGTGCTGAGAGG
GCGTATGTGTGTCAATTGCCCTTTGGGAACCTATTATAATCTGGAACATTTCACCTGTGAAAGCTGCC
GGATCGGATCCTATCAAGATGAAGAAGGGCAACTTGAGTGCAAGCTTTGCCCCTCTGGGATGTACACG
GAATATATCCATTCAAGAAACATCTCTGATTGTAAAGCTCAGTGTAAACAAGGCACCTACTCATACAG
TGGACTTGAGACTTGTGAATCGTGTCCACTGGGCACTTATCAGCCAAAATTTGGTTCCCGGAGCTGCC
TCTCGTGTCCAGAAAACACCTCAACTGTGAAAAGAGGAGCCGTGAACATTTCTGCATGTGGAGTTCCT
TGTCCAGAAGGAAAATTCTCGCGTTCTGGGTTAATGCCCTGTCACCCATGTCCTCGTGACTATTACCA
ACCTAATGCAGGGAAGGCCTTCTGCCTGGCCTGTCCCTTTTATGGAACTACCCCATTCGCTGGTTCCA
GATCCATCACAGAATGTTCAAGTTTTAGTTCAACTTTCTCAGCGGCAGAGGAAAGTGTGGTGCCCCCT
GCCTCTCTTGGACATATTAAAAAGAGGCATGAAATCAGCAGTCAGGCAAGTCATGAATGCTTCTTTAA
CCCTTGCCACAATAGTGGAACCTGCCAGCAACTTGGGCGTGGTTATGTTTGTCTCTGTCCACTTGGAT
ATACAGGTTTAAAGTGTGAAACAGACATCGATGAGTGCAGCCCACTGCCTTGCCTCAACAATGGAGTT
TGTAAAGACCTAGTTGGGGAATTCATTTGTGAGTGCCCATCAGGTTACACAGGTAAGCACTGTGAATT
GAACATCAATGAATGTCAGTCTAATCCATGTAGAAATCAGGCCACCTGTGTGGATGAATTAAATTCAT
ACAGTTGTAAATGTCAGCCAGGATTTTCAGGCAAAAGGTGTGAAACAGGTATGTATCAACTCAGTGTT
ATTAATAACCTTAATAATGCAGTCTGTGAAGACCAGGTTGGGGGATTCTTGTGCAAATGCCCACCTGG
ATTTTTGGGTACCCGATGTGGAAAGAACGTCGATGAGTGTCTCAGTCAGCCATGCAAAAATGGAGCTA
CCTGTAAAGACGGTGCCAATAGCTTCAGGTGCCTGTGTGCAGCTGGCTTCACAGGATCACACTGTGAA
TTGAACATCAATGAATGTCAGTCTAATCCATGTAGAAATCAGGCCACCTGTGTGGATGAATTAAATTC
ATACAGTTGTAAATGTCAGCCAGGATTTTCAGGCAAAAGGTGTGAAACAGAACAGTCTACAGGCTTTA
ACCTGGATTTTGAAGTTTCTGGCATCTATGGATATGTCATGCTAGATGGCATGCTCCCATCTCTCCAT
GCTCTAACCTGTACCTTCTGGATGAAATCCTCTGACGACATGAACTATGGAACACCAATCTCCTATGC
AGTTGATAACGGCAGCGACAATACCTTGCTCCTGACTGATTATAACGGGTGGGTTCTTTATGTGAATG
GCAGGGAAAAGATAACAAACTGTCCCTCGGTGAATGATGGCAGATGGCATCATATTGCAATCACTTGG
ACAAGTACTGGTGGAGCCTGGAGGGTCTATATAAATGGGGAATTATCTGACGGTGGTACTGGCCTCTC
CATTGGCAAAGCCATACCTGGTGGCGGTGCATTAGTTCTTGGGCAAGAGCAAGACAAAAAAGGAGAGG
GGTTCAACCCGGCTGAGTCTTTTGTGGGCTCCATAAGCCAGCTCAACCTCTGGGACTATGTCCTGTCT
CCACAGCAGGTGAAGTCACTGGCTACCTCCTGCCCAGAGGAACTCAGTAAAGGAAACGTGTTAGCATG
GCCTGATTTCTTGTCAGGAATTGTGGGGAAAGTGAAGATCGATTCTAAGAGCATATTTTGTTCTGATT
GCCCACGCTTGGGAGGGTCAGTGCCTCATCTGAGAACTGCATCTGAAGATTTAAAACCAGGTTCCAAA
GTCAATCTGTTCTGTGAACCAGGCTTCCAGCTGGTCGGGAACCCTGTGCAGTACTGTCTGAATCAAGG
ACAGTGGACACAACCACTCCCCCACTGTGAACGCATTCGCTGTGGGGTGCCACCTCCTTTGGAGAATG
GCTTCCATTCAGCCGATGACTTCTATGCTGGCAGCACAGTAACCTACCAGTGCAACAATGGCTACTAT
CTATTGGGTGACTCAAGGATGTTCTGTACAGATAATGGGAGCTGGAACGGCGTTTCACCATCCTGCTT
AGATGTCGATGAGTGTGCAGTTGGATCAGATTGTAGTGAGCATGCTTCTTGCCTGAACGTAGATGGAT
CCTACATATGTTCATGTGTCCCACCGTACACAGGAGATGGGAAAAACTGTGCAGAACCTATAAAATGT
AAGGCTCCAGGAAATCCGGAAAATGGCCACTCCTCAGGTGAGATTTATACAGTAGGTGCCGAAGTCAC
ATTTTCGTGTCAGGAAGGATACCAGTTGATGGGAGTAACCAAAATCACATGTTTGGAGTCTGGAGAAT
GGAATCATCTAATACCATATTGTAAAGCTGTTTCATGTGGTAAACCGGCTATTCCAGAAAATGGTTGC
ATTGAGGAGTTAGCATTTACTTTTGGCAGCAAAGTGACATATAGGTGTAATAAAGGATATACTCTGGC
CGGTGATAAAGAATCATCCTGTCTTGCTAACAGTTCTTGGAGTCATTCCCCTCCTGTGTGTGAACCAG
TGAAGTGTTCTAGTCCGGAAAATATAAATAATGGAAAATATATTTTGAGTGGGCTTACCTACCTTTCT
ACTGCATCATATTCATGCGATACAGGATACAGCTTACAGGGCCCTTCCATTATTGAATGCACGGCTTC
TGGCATCTGGGACAGAGCGCCACCTGCCTGTCACCTCGTCTTCTGTGGAGAACCACCTGCCATCAAAG
ATGCTGTCATTACGGGGAATAACTTCACTTTCAGGAACACCGTCACTTACACTTGCAAAGAAGGCTAT
ACTCTTGCTGGTCTTGACACCATTGAATGCCTGGCCGACGGCAAGTGGAGTAGAAGTGACCAGCAGTG
CCTGGCTGTCTCCTGTGATGAGCCACCCATTGTGGACCACGCCTCTCCAGAGACTGCCCATCGGCTCT
TTGGAGACATTGCATTCTACTACTGCTCTGATGGTTACAGCCTAGCAGAGACTTCCCAGCTTCTCTGC
AATGCCCAGGGCAAGTGGGTACCCCCAGAAGGTCAAGACATGCCCCGTTGTATAGCTCATTTCTGTGA
AAAACCTCCATCGGTTTCCTATAGCATCTTGGAATCTGTGAGCAAAGCAAAATTTGCAGCTGGCTCAG
TTGTGAGCTTTAAATGCATGGAAGGCTTTGTACTGAACACCTCAGCAAAGATTGAATGTATGAGAGGT
CATGAATGGCTATGCAAGTGGATCAAACTACAGTTTTGGAGCCATGGTGGCTTACAGCTGCAACAAGG
GGTTCTACATCAAAGGGGAAAAGAAGAGCACCTGCGAAGCCACAGGGCAGTGGAGTAGTCCTATACCG
ACGTGCCACCCGGTATCTTGTGGTGAACCACCTAAGGTTGAGAATGGCTTTCTGGAGCATACAACTGG
CAGGATCTTTGAGAGTGAAGTGAGGTATCAGTGTAACCCGGGCTATAAGTCAGTCGGAAGTCCTGTAT
TTGTCTGCCAAGCCAATCGCCACTGGCACAGTGAATCCCCTCTGATGTGTGTTCCTCTCGACTGTGGA
AAACCTCCCCCGATCCAGAATGGCTTCATGAAAGGAGAAAACTTTGAAGTAGGGTCCAAGGTTCAGTT
TTTCTGTAATGAGGGTTATGAGCTTGTTGGTGACAGTTCTTGGACATGTCAGAAATCTGGCAAATGGA
ATAAGAAGTCAAATCCAAAGTGCATGCCTGCCAAGTGCCCAGAGCCGCCCCTCTTGGAAAACCAGCTA
GTATTAAAGGAGTTGACCACCGAGGTAGGAGTTGTGACATTTTCCTGTAAAGAAGGGCATGTCCTGCA
AGGCCCCTCTGTCCTGAAATGCTTGCCATCCCAGCAATGGAATGACTCTTTCCCTGTTTGTAAGATTG
TTCTTTGTACCCCACCTCCCCTAATTTCCTTTGGTGTCCCCATTCCTTCTTCTGCTCTTCATTTTGGA
AGTACTGTCAAGTATTCTTGTGTAGGTGGGTTTTTCCTAAGAGGAAATTCTACCACCCTCTGCCAACC
TGATGGCACCTGGAGCTCTCCACTGCCAGAATGTGTTCCAGTAGAATGTCCCCAACCTGAGGAAATCC
CCAATGGAATCATTGATGTGCAAGGCCTTGCCTATCTCAGCACAGCTCTCTATACCTGCAAGCCAGGC
TTTGAATTGGTGGGAAATACTACCACCCTTTGTGGAGAAAATGGTCACTGGCTTGGAGGAAAACCAAC
ATGTAAAGCCATTGAGTGCCTGAAACCCAAGGAGATTTTGAATGGCAAATTCTCTTACACGGACCTAC
ACTATGGACAGACCGTTACCTACTCTTGCAACCGAGGCTTTCGGCTCGAAGGTCCCAGTGCCTTGACC
TGTTTAGAGACAGGTGATTGGGATGTAGATGCCCCATCTTGCAATGCCATCCACTGTGATTCCCCACA
ACCCATTGAAAATGGTTTTGTAGAAGGTGCAGATTACAGCTATGGTGCCATAATCATCTACAGTTGCT
TCCCTGGGTTTCAGGTGGCTGGTCATGCCATGCAGACCTGTGAAGAGTCAGGATGGTCAAGTTCCATC
CCAACATGTATGCCAATAGACTGTGGCCTCCCTCCTCATATAGATTTTGGAGACTGTACTAAACTCAA
AGATGACCAGGGATATTTTGAGCAAGAAGACGACATGATGGAAGTTCCATATGTGACTCCTCACCCTC
CTTATCATTTGGGAGCAGTGGCTAAAACCTGGGAAAATACAAAGGAGTCTCCTGCTACACATTCATCA
AACTTTCTGTATGGTACCATGGTTTCATACACCTGTAATCCAGGATATGAACTTCTGGGGAACCCTGT
GCTGATCTGCCAGGAAGATGGAACTTGGAATGGCAGTGCACCATCCTGCATTTCAATTGAATGTGACT
TGCCTACTGCTCCTGAAAATGGCTTTTTGCGTTTTACAGAGACTAGCATGGGAAGTGCTGTGCAGTAT
AGCTGTAAACCTGGACACATTCTAGCAGGCTCTGACTTAAGGCTTTGTCTAGAGAATAGAAAGTGGAG
TGGTGCCTCCCCACGCTGTGAAGCCATTTCATGCAAAAAGCCAAATCCAGTCATGAATGGATCCATCA
AAGGAAGCAACTACACATACCTGAGCACGTTGTACTATGAGTGTGACCCCGGATATGTGCTGAATGGC
ACTGAGAGGAGAACATGCCAGGATGACAAAAACTGGGATGAGGATGAGCCCATTTGCATTCCTGTGGA
CTGCAGTTCACCCCCAGTCTCAGCCAATGGCCAGGTGAGAGGAGACGAGTACACATTCCAAAAAGAGA
TTGAATACACTTGCAATGAAGGGTTCTTGCTTGAGGGAGCCAGGAGTCGGGTTTGTCTTGCCAATGGA
AGTTGGAGTGGAGCCACTCCCGACTGTGTGCCTGTCAGATGTGCCACCCCGCCACAACTGGCCAATGG
GGTGACGGAAGGCCTGGACTATGGCTTCATGAAGGAAGTAACATTCCACTGTCACGAGGGCTACATCT
TGCACGGTGCTCCAAAACTCACCTGTCAGTCAGATGGCAACTGGGATGCAGAGATTCCTCTCTGTAAA
CCAGTCAACTGTGGACCTCCTGAAGATCTTGCCCATGGTTTCCCTAATGGTTTTTCCTTTATTCATGG
GGGCCATATACAGTATCAGTGCTTTCCTGGTTATAAGCTCCATGGAAATTCATCAAGAAGGTGCCTCT
CCAATGGCTCCTGGAGTGGCAGCTCACCTTCCTGCCTGCCTTGCAGATGTTCCACACCAGTAATTGAA
TATGGAACTGTCAATGGGACAGATTTTGACTGTGGAAAGGCAGCCCGGATTCAGTGCTTCAAAGGCTT
CAAGCTCCTAGGACTTTCTGAAATCACCTGTGAAGCCGATGGCCAGTGGAGCTCTGGGTTCCCCCACT
GTGAACACACTTCTTGTGGTTCTCTTCCAATGATACCAAATGCGTTCATCAGTGAGACCAGCTCTTGG
AAGGAAAATGTGATAACTTACAGCTGCAGGTCTGGATATGTCATACAAGGCAGTTCAGATCTGATTTG
TACAGAGAAAGGGGTATGGAGCCAGCCTTATCCAGTCTGTGAGCCCTTGTCCTGTGGGTCCCCACCGT
CTGTCGCCAATGCAGTGGCAACTGGAGAGGCACACACCTATGAAAGTGAAGTGAAACTCAGATGTCTG
GAAGGTTATACGATGGATACAGATACAGATACATTCACCTGTCAGAAAGATGGTCGCTGGTTCCCTGA
GAGAATCTCCTGCAGTCCTAAAAAATGTCCTCTCCCGGAAAACATAACACATATACTTGTACATGGGG
ACGATTTCAGTGTGAATAGGCAAGTTTCTGTGTCATGTGCAGAAGGGTATACCTTTGAGGGAGTTAAC
ATATCAGTATGTCAGCTTGATGGAACCTGGGAGCCACCATTCTCCGATGAATCTTGCAGTCCAGTTTC
TTGTGGGAAACCTGAAAGTCCAGAACATGGATTTGTGGTTGGCAGTAAATACACCTTTGAAAGCACAA
TTATTTATCAGTGTGAGCCTGGCTATGAACTAGAGGGGAACAGGGAACGTGTCTGCCAGGAGAACAGA
CAGTGGAGTGGAGGGGTGGCAATATGCAAAGAGACCAGGTGTGAAACTCCACTTGAATTTCTCAATGG
GAAAGCTGACATTGAAAACAGGACGACTGGACCCAACGTGGTATATTCCTGCAACAGAGGCTACAGTC
TTGAAGGGCCATCTGAGGCACACTGCACAGAAAATGGAACCTGGAGCCACCCAGTCCCTCTCTGCAAA
CCAAATCCATGCCCTGTTCCTTTTGTGATTCCCGAGAATGCTCTGCTGTCTGAAAAGGAGTTTTATGT
TGATCAGAATGTGTCCATCAAATGTAGGGAAGGTTTTCTGCTGCAGGGCCACGGCATCATTACCTGCA
ACCCCGACGAGACGTGGACACAGACAAGCGCCAAATGTGAAAAAATCTCATGTGGTCCACCAGCTCAC
GTAGAAAATGCAATTGCTCGAGGCGTACATTATCAATATGGAGACATGATCACCTACTCATGTTACAG
TGGATACATGTTGGAGGGTTTCCTGAGGAGTGTTTGTTTAGAAAATGGACAATGGACATCACCTCCTA
TTTGCAGAGCTGTCTGTCGATTTCCATGTCAGAATGGGGGCATCTGCCAACGCCCAAATGCTTGTTCC
TGTCCAGAGGGCTGGATGGGGCGCCTCTGTGAAGAACCAATCTGCATTCTTCCCTGTCTGAACGGAGG
TCGCTGTGTGGCCCCTTACCAGTGTGACTGCCCGCCTGGCTGGACGGGGTCTCGCTGTCATACAGCTG
TTTGCCAGTCTCCCTGCTTAAATGGTGGALAATGTGTAAGACCAAACCGATGTCACTGTCTTTCTTCT
TGGACGGGACATAACTGTTCCAGGAAAAGGAGGACTGGGTTTTAACCACTGCACGACCATCTGGCTCT
CCCAAAAGCAGGATCATCTCTCCTCGGTAGTGCCTGGGCATCCTGGAACTTATGCAAAGAAAGTCCAA
CATGGTGCTGGGTCTTGTTTAGTAAACTTGTTACTTGGGGTTACTTTTTTTATTTTGTGATATATTTT
GTTATTCCTTGTGACATACTTTCTTACATGTTTCCATTTTTAAATATGCCTGTATTTTCTATATAAAA
ATTATATTAAATAGATGCTGCTCTACCCTCACAAAATGTACATATTCTGCTGTCTATTGGGAAAGTTC
CTGGTACACATTTTTATTCAGTTACTTAAAATGATTTTTCCATTAAAGTATATTTTGCTACTAAATAA
AAAAAA

| | | |
|---|---|---|
| NOV8l, SNP13374702 of | SEQ ID NO: 200 | MW at 390031.5kD |

| | | | |
|---|---|---|---|
| CG50646-04, Protein Sequence | SNP Pos: 794 | 3570 aa | SNP Change: His to Arg |

MRRICAACWGLALVSGWATFQQMSPSRNFSFRLFPETAPGAPGSIPAPPAPGDEAAGSRVERLGQAFR
VRLLRELSERLELVFLVDDSSSVGEVNFRSELMFVRKLLSDFPVVPTATRVAIVTFSSKNYVVPRVDY
ISTRRARQHKCALLLQEIPAISYRGGGTYTKGAFQQAAQILLHARENSTKVVFLITDGYSNGGDPRPI
AASLRDSGVEIFTFGIWQGNIRELNDMASTPKEEHCYLLHSFEEFEALVALCHMLFVDLPSGSFIQDD
MVHCSYLCDEGKDCCDRMGSCKCGKHTGHFECICEKGYNGKGLQYDCTVCPSGTYKPEGSPGGISSCI
PCPDENHTSPPGSTSPEDCVCREGYPASGQTCEVVHCPALKPPENGYFIQNTCNNHFNAACGVRCHPG
FDLVGSSIILCLPNGLWSGSESYCRVRTCPHLRQPKHGHISCSTREMLYKTTCLVACDEGYRLEGSDK
LTCQGNSQWDGPEPRCVERHCSTFQMPKDVIISPHNCGKQPAKFGTICYVSCRQGFILSGVKEMLRCT
TSGKWNVGVQAAVCKDVEAPQINCPKDIEAKTLEQQDSANVTWQIPTAKDNSGEKVSVHVHPAFTPPY
LFPIGDVAIVYTATDLSGNQASCIFHIKVIDAEPPVIDWCRSPPPVQVSEKVHAASWDEPQFSDNSGA
ELVITRSHTQGDLFPQGETIVQYTATDPSGNNRTCDIHIVIKGSPCEIPFTPVNGDFICTPDNTGVNC
TLTCLEGYDFTEGSTDKYYCAYEDGVWKPTYTTEWPDCASKRFANRGFKSFEMFYKAARCDDTDLMKK
FSEAFETTLGKMVPSFCSDAEDIDCPLEENLTKKYCLEYNYDYENGFAIGPGGWGAANRLDYSYDDFL
DTVQETATSIGNAKSSRIKRSAPLSDYKIKLIFNITASVPLPDERNDTLEWENQQRLLQTLETITNKL
KRTLNKDPMYSFQLASEILIADSNSLETKKASPFCRPGSVLRGRMCVNCPLGTYYNLEHFTCESCRIG
SYQDEEGQLECKLCPSGMYTEYIHSRNISDCKAQCKQGTYSYSGLETCESCPLGTYQPKFGSRSCLSC
TECSSFSSTFSAAEESVVPPASLGHIKKRHEISSQASHECFFNPCHNSGTCQQLGRGYVCLCPLGYTG
LKCETDIDECSPLPCLNNGVCKDLVGEFICECPSGYTGKHCELNINECQSNPCRNQATCVDELNSYSC
KCQPGFSGKRCETGMYQLSVINNLNNAVCEDQVGGFLCKCPPGFLGTRCGKNVDECLSQPCKNGATCK
DGANSFRCLCAAGFTGSHCELNINECQSNPCRNQATCVDELNSYSCKCQPGFSGKRCETEQSTGFNLD
FEVSGIYGYVMLDGMLPSLHALTCTFWMKSSDDMNYGTPISYAVDNGSDNTLLLTDYNGWVLYVNGRE
KITNCPSVNDGRWHHIAITWTSTGGAWRVYINGELSDGGTGLSIGKAIPGGGALVLGQEQDKKGEGFN
PAESFVGSISQLNLWDYVLSPQQVKSLATSCPEELSKGNVLAWPDFLSGIVGKVKIDSKSIFCSDCPR
LGGSVPHLRTASEDLKPGSKVNLFCEPGFQLVGNPVQYCLNQGQWTQPLPHCERIRCGVPPPLENGFH
SADDFYAGSTVTYQCNNGYYLLGDSRMFCTDNGSWNGVSPSCLDVDECAVGSDCSEHASCLNVDGSYI
CSCVPPYTGDGKNCAEPIKCKAPGNPENGHSSGEIYTVGAEVTFSCQEGYQLMGVTKITCLESGEWNH
LIPYCKAVSCGKPAIPENGCIEELAFTFGSKVTYRCNKGYTLAGDKESSCLANSSWSHSPPVCEPVKC
SSPENINNGKYILSGLTYLSTASYSCDTGYSLQGPSIIECTASGIWDRAPPACHLVFCGEPPAIKDAV
ITGNNFTFRNTVTYTCKEGYTLAGLDTIECLADGKWSRSDQQCLAVSCDEPPIVDHASPETAHRLFGD
IAFYYCSDGYSLADNSQLLCNAQGKWVPPEGQDMPRCIAHFCEKPPSVSYSILESVSKAKFAAGSVVS
FKCMEGFVLNTSAKIECMRGGQWNPSPMSIQCIPVRCGEPPSIMNGYASGSNYSFGAMVAYSCNKGFY
IKGEKKSTCEATGQWSSPIPTCHPVSCGEPPKVENGFLEHTTGRIFESEVRYQCNPGYKSVGSPVFVC
QANRHWHSESPLMCVPLDCGKPPPIQNGFMKGENFEVGSKVQFFCNEGYELVGDSSWTCQKSGKWNKK
SNPKCMPAKCPEPPLLENQLVLKELTTEVGVVTFSCKEGHVLQGPSVLKCLPSQQWNDSFPVCKIVLC
TPPPLISFGVPIPSSALHFGSTVKYSCVGGFFLRGNSTTLCQPDGTWSSPLPECVPVECPQPEEIPNG
IIDVQGLAYLSTALYTCKPGFELVGNTTTLCGENGHWLGGKPTCKAIECLKPKEILNGKFSYTDLHYG
QTVTYSCNRGFRLEGPSALTCLETGDWDVDAPSCNAIHCDSPQPIENGFVEGADYSYGAIIIYSCFPG
FQVAGHAMQTCEESGWSSSIPTCMPIDCGLPPHIDFGDCTKLKDDQGYFEQEDDNMEVPYVTPHPPYH
LGAVAKTWENTKESPATHSSNFLYGTMVSYTCNPGYELLGNPVLICQEDGTWNGSAPSCISIECDLPT
APENGFLRFTETSMGSAVQYSCKPGHILAGSDLRLCLENRKWSGASPRCEAISCKKPNPVMNGSIKGS
NYTYLSTLYYECDPGYVLNGTERRTCQDDKNWDEDEPICIPVDCSSPPVSANGQVRGDEYTFQKEIEY
TCNEGFLLEGARSRVCLANGSWSGATPDCVPVRCATPPQLANGVTEGLDYGFMKEVTFHCHEGYILHG
APKLTCQSDGNWDAEIPLCKPVNCGPPEDLAHGFPNGFSFIHGGHIQYQCFPGYKLHGNSSRRCLSNG
SWSGSSPSCLPCRCSTPVIEYGTVNGTDFDCGKAARIQCFKGFKLLGLSEITCEADGQWSSGFPHCEH
TSCGSLPMIPNAFISETSSWKENVITYSCRSGYVIQGSSDLICTEKGVWSQPYPVCEPLSCGSPPSVA
NAVATGEAHTYESEVKLRCLEGYTMDTDTDTFTCQKDGRWFPERISCSPKKCPLPENITHILVHGDDF
SVNRQVSVSCAEGYTFEGVNISVCQLDGTWEPPFSDESCSPVSCGKFESPEHGFVVGSKYTFESTIIY
QCEPGYELEGNRERVCQENRQWSGGVAICKETRCETPLEFLNGKADIENRTTGPNVVYSCNRGYSLEG
PSEAHCTENGTWSHPVPLCKPNPCPVPFVIPENALLSEKEFYVDQNVSIKCREGFLLQGHGIITCNPD
ETWTQTSAKCEKISCGPPAHVENAIARGVHYGYGDMITYSCYSGYMLEGFLRSVCLENGTWTSPPICR
AVCRFPCQNGGICQRPNACSCPEGWMGRLCEEPICILPCLNGGRCVAPYQCDCPPGWTGSRCHTAVCQ
SPCLNGGKCVRPNRCHCLSSWTGHNCSRKRRTGF

| | |
|---|---|
| SEQ ID NO: 201 | 11158 bp |

| | | |
|---|---|---|
| NOV8m, SNP13374257 of | ORF Start: ATG at 77 | ORF Stop: TAA at 10787 |

| | | |
|---|---|---|
| CG50646-04, DNA Sequence | SNP Pos: 7217 | SNP Change: A to G |

CAATTGGTCTAGGGTCTCCCCCATTGGAATATCCATCAGTGATGAGAAATACAACGTTTGTTGAGTTT
TCTCTAGCATGAGAAGAATTTGCGCGGCTTGCTGGGGTCTGGCGCTCGTTTCGGGCTGGGCGACCTTT
CAGCAGATGTCCCCGTCGCGCAATTTCAGCTTCCGCCTCTTCCCCGAGACCGCGCCCGGGGCCCCCGG
GAGTATCCCCGCGCCGCCCGCTCCTGGCGACGAAGCGGCGGGGAGCAGAGTGGAGCGGCTGGGCCAGG
CGTTCCGCGTGCGGCTGCTGCGGGAGCTCAGCGAGCGCCTGGAGCTTGTCTTCCTGGTGGATGATTCG
TCCAGCGTGGGCGAAGTCAACTTCCGCAGCGAGCTCATGTTCGTCCGCAAGCTGCTGTCCGACTTCCC
CGTGGTGCCCACGGCCACGCGCGTGGCCATCGTGACCTTCTCGTCCAAGAACTACGTGGTGCCGCGCG
TCGATTACATCTCCACCCGCCGCGCGCGCCAGCACAAGTGCGCGCTGCTCCTCCAAGAGATCCCTGCC
ATCTCCTACCGAGGTGGCGGCACCTACACCAAGGGCGCCTTCCAGCAAGCCGCGCAAATTCTTCTTCA
TGCTAGAGAAAACTCAACAAAAGTTGTATTTCTCATCACTGATGGATATTCCAATGGGGGAGACCCTA
GACCAATTGCAGCGTCACTGCGAGATTCAGGAGTGGAGATCTTCACTTTTGGCATATGGCAAGGGAAC
ATTCGAGAGCTGAATGACATGGCTTCCACCCCAAAGGAGGAGCACTGTTACCTGCTACACAGTTTTGA
AGAATTTGAGGCTTTAGTCGCCCTCTGTCATATGTTATTTGTAGATCTACCTTCTGGGAGTTTTATTC
AAGATGATATGGTCCACTGCTCATATCTTTGTGATGAAGGCAAGGACTGCTGTGACCGAATGGGAAGC
TGCAAATGTGGGAAACACACAGGCCATTTTGAGTGCATCTGTGAAAAGGGGTATAACGGGAAAGGTCT
GCAGTATGACTGCACAGTTTGCCCATCGGGGACATACAAACCTGAAGGCTCACCAGGAGGAATCAGCA
GTTGCATTCCATGTCCTGATGAAAATCACACCTCTCCACCTGGAAGCACATCCCCTGAAGACTGTGTC
TGCAGAGAGGGATACAGGGCATCTGGCCAGACCTGTGAAGTTGTCCACTGCCCTGCCCTGAAGCCTCC
CGAAAATGGTTACTTTATCCAAAACACTTGCAACAACCACTTCAATGCAGCCTGTGGGGTCCGATGTC
ACCCTGGATTTGATCTTGTGGGAAGCAGCATCATCTTATGTCTACCCAATGGTTTGTGGTCCGGTTCA
GAGAGCTACTGCAGAGTAAGAACATGTCCTCATCTCCGCCAGCCGAAACATGGCCACATCAGCTGTTC
TACAAGGGAAATGTTATATAAGACAACATGTTTGGTTGCCTGTGATGAAGGGTACAGGCTAGAAGGCA
GTGATAAGCTTACTTGTCAAGGAAACAGCCAGTGGGATGGGCCAGAACCCCGGTGTGTGGAGCGCCAC
TGTTCCACCTTTCAGATGCCCAAAGATGTCATCATATCCCCCCACAACTGTGGCAAGCAGCCAGCCAA
ATTTGGGACGATCTGCTATGTAAGTTGCCGCCAAGGGTTCATTTTATCTGGAGTCAAAGAAATGCTGA
GATGTACCACTTCTGGAAAATGGAATGTCGGAGTTCAGGCAGCTGTGTGTAAAGACGTGGAGGCTCCT
CAAATCAACTGTCCTAAGGACATAGAGGCTAAGACTCTGGAACAGCAAGATTCTGCCAATGTTACCTG
GCAGATTCCAACAGCTAAAGACAACTCTGGTGAAAAGGTGTCAGTCCACGTTCATCCAGCTTTCACCC
CACCTTACCTTTTCCCAATTGGAGATGTTGCTATCGTATACACGGCAACTGACCTATCCGGCAACCAG
GCCAGCTGCATTTTCCATATCAAGGTTATTGATGCAGAACCACCTGTCATAGACTGGTGCAGATCTCC
ACCTCCCGTCCAGGTCTCGGAGAAGGTACATGCCGCAAGCTGGGATGAGCCTCAGTTCTCAGACAACT
CAGGTGCTGAATTGGTCATTACCAGAAGTCATACACAAGGAGACCTTTTCCCTCAAGGGGAGACTATA
GTACAGTATACAGCCACTGACCCCTCAGGCAATAACAGGACATGTGATATCCATATTGTCATAAAAGG
TTCTCCCTGTGAAATTCCATTCACACCTGTAAATGGGGATTTTATATGCACTCCAGATAATACTGGAG
TCAACTGTACATTAACTTGCTTGGAGGGCTATGATTTCACAGAAGGGTCTACTGACAAGTATTATTGT
GCTTATGAAGATGGCGTCTGGAAACCAACATATACCACTGAATGGCCAGACTGTGCCAGTAAGCGTTT
TGCAAACCACGGGTTCAAGTCCTTTGAGATGTTCTACAAAGCAGCTCGTTGTGATGACACAGATCTGA
TGAAGAAGTTTTCTGAAGCATTTGAGACGACCCTGGGAAAAATGGTCCCATCATTTTGTAGTGATGCA
GAGGACATTGACTGCAGACTGGAGGAGAACCTGACCAAAAAATATTGCCTAGAATATAATTATGACTA
TGAAAATGGCTTTGCAATTGGTCCAGGTGGCTGGGGTGCAGCTAATAGGCTGGATTACTCTTACGATG
ACTTCCTGGACACTGTGCAAGAAACAGCCACAAGCATCGGCAATGCCAAGTCCTCACGGATTAAAAGA
AGTGCCCCATTATCTGACTATAAAATTAAGTTAATTTTTAACATCACAGCTAGTGTGCCATTACCCGA
TGAAAGAAATGATACCCTTGAATGGGAAAATCAGCAACGACTCCTTCAGACATTGGAAACTATCACAA
ATAAACTGAAAAGGACTCTCAACAAAGACCCCATGTATTCCTTTCAGCTTGCATCAGAAATACTTATA
GCCGACAGCAATTCATTAGAAACAAAAAAGGCTTCCCCCTTCTGCAGACCAGGCTCAGTGCTGAGAGG
GCGTATGTGTGTCAATTGCCCTTTGGGAACCTATTATAATCTGGAACATTTCACCTGTGAAAGCTGCC
GGATCGGATCCTATCAAGATGAAGAAGGGCAACTTGAGTGCAAGCTTTGCCCCTCTGGGATGTACACG
GAATATATCCATTCAAGAAACATCTCTGATTGTAAAGCTCAGTGTAAACAAGGCACCTACTCATACAG
TGGACTTGAGACTTGTGAATCGTGTCCACTGGGCACTTATCAGCCAAAATTTGGTTCCCGGAGCTGCC
TCTCGTGTCCAGAAAACACCTCAACTGTGAAAAGAGGAGCCGTGAACATTTCTGCATGTGGAGTTCCT
TGTCCAGAAGGAAAATTCTCGCGTTCTGGGTTAATGCCCTGTCACCCATGTCCTCGTGACTATTACCA
ACCTAATGCAGGGAAGGCCTTCTGCCTGGCCTGTCCCTTTTATGGAACTACCCCATTCGCTGGTTCCA
GATCCATCACAGAATGTTCAAGTTTTAGTTCAACTTTCTCAGCGGCAGAGGAAAGTGTGGTGCCCCCT
GCCTCTCTTGGACATATTAAAAAGAGGCATGAAATCAGCAGTCAGGCAAGTCATGAATGCTTCTTTAA
CCCTTGCCACAATAGTGGAACCTGCCAGCAACTTGGGCGTGGTTATGTTTGTCTCTGTCCACTTGGAT
ATACAGGTTTAAAGTGTGAAACAGACATCGATGAGTGCAGCCCACTGCCTTGCCTCAACAATGGAGTT
TGTAAAGACCTAGTTGGGGAATTCATTTGTGAGTGCCCATCAGGTTACACAGGTAAGCACTGTGAATT
GAACATCAATGAATGTCAGTCTAATCCATGTAGAAATCAGGCCACCTGTGTGGATGAATTAAATTCAT
ACAGTTGTAAATGTCAGCCAGGATTTTCAGGCAAAGGTGTGAAAACAGGTATGTATCAACTCAGTGTT
ATTAATAACCTTAATAATGCAGTCTGTGAAGACCAGGTTGGGGGATTCTTGTGCAAATGCCCACCTGG
ATTTTTGGGTACCCGATGTGGAAAGAACGTCGATGAGTGTCTCAGTCAGCCATGCAAAAATGGAGCTA
CCTGTAAAGACGGTGCCAATAGCTTCAGGTGCCTGTGTGCAGCTGGCTTCACAGGATCACACTGTGAA
TTGAACATCAATGAATGTCAGTCTAATCCATGTAGAAATCAGGCCACCTGTGTGGATGAATTAAATTC
ATACAGTTGTAAATGTCAGCCAGGATTTTCAGGCAAAAGGTGTGAAACAGAACAGTCTACAGGCTTTA
ACCTGGATTTTGAAGTTTCTGGCATCTATGGATATGTCATGCTAGATGGCATGCTCCCATCTCTCCAT
GCTCTAACCTGTACCTTCTGGATGAAATCCTCTGACGACATGAACTATGGAACACCAATCTCCTATGC
AGTTGATAACGGCAGCGACAATACCTTGCTCCTGACTGATTATAACGGGTGGGTTCTTTATGTGAATG
GCAGGGAAAAGATAACAAACTGTCCCTCGGTGAATGATGGCAGATGGCATCATATTGCAATCACTTGG
ACAAGTACTGGTGGAGCCTGGAGGGTCTATATAAATGGGGAATTATCTGACGGTGGTACTGGCCTCTC
CATTGGCAAAGCCATACCTGGTGGCGGTGCATTAGTTCTTGGGCAAGAGCAAGACAAAAAAGGAGAGG
GGTTCAACCCGGCTGAGTCTTTTGTGGGCTCCATAAGCCAGCTCAACCTCTGGGACTATGTCCTGTCT
CCACAGCAGGTGAAGTCACTGGCTACCTCCTGCCCAGAGGAACTCAGTAAAGGAAACGTGTTAGCATG
GCCTGATTTCTTGTCAGGAATTGTGGGGAAAGTGAAGATCGATTCTAAGAGCATATTTTGTTCTGATT
GCCCACGCTTGGGAGGGTCAGTGCCTCATCTGAGAACTGCATCTGAAGATTTAAAACCAGGTTCCAAA
GTCAATCTGTTCTGTGAACCAGGCTTCCAGCTGGTCGGGAACCCTGTGCAGTACTGTCTGAATCAAGG
ACAGTGGACACAACCACTCCCCCACTGTGAACGCATTCGCTGTGGGGTGCCACCTCCTTTGGAGAATG
GCTTCCATTCAGCCGATGACTTCTATGCTGGCAGCACAGTAACCTACCAGTGCAACAATGGCTACTAT
CTATTGGGTGACTCAAGGATGTTCTGTACAGATAATGGGAGCTGGAACGGCGTTTCACCATCCTGCTT
AGATGTCGATGAGTGTGCAGTTGGATCAGATTGTAGTGAGCATGCTTCTTGCCTGAACGTAGATGGAT
CCTACATATGTTCATGTGTCCCACCGTACACAGGAGATGGGAAAAACTGTGCAGAACCTATAAAATGT
AAGGCTCCAGGAAATCCGGAAAATGGCCACTCCTCAGGTGAGATTTATACAGTAGGTGCCGAAGTCAC
ATTTTCGTGTCAGGAAGGATACCAGTTGATGGGAGTAACCAAAATCACATGTTTGGAGTCTGGAGAAT
GGAATCATCTAATACCATATTGTAAAGCTGTTTCATGTGGTAAACCGGCTATTCCAGAAAATGGTTGC
ATTGAGGAGTTAGCATTTACTTTTGGCAGCAAAGTGACATATAGGTGTAATAAAGGATATACTCTGGC
CGGTGATAAAGAATCATCCTGTCTTGCTAACAGTTCTTGGAGTCATTCCCCTCCTGTGTGTGAACCAG
TGAAGTGTTCTAGTCCGGAAAATATAAATAATGGAAAATATATTTTGAGTGGGCTTACCTACCTTTCT
ACTGCATCATATTCATGCGATACAGGATACAGCTTACAGGGCCCTTCCATTATTGAATGCACGGCTTC
TGGCATCTGGGACAGAGCGCCACCTGCCTGTCACCTCGTCTTCTGTGGAGAACCACCTGCCATCAAAG
ATGCTGTCATTACGGGGAATAACTTCACTTTCAGGAACACCGTCACTTACACTTGCAAAGAAGGCTAT
ACTCTTGCTGGTCTTGACACCATTGAATGCCTGGCCGACGGCAAGTGGAGTAGAAGTGACCAGCAGTG
CCTGGCTGTCTCCTGTGATGAGCCACCCATTGTGGACCACGCCTCTCCAGAGACTGCCCATCGGCTCT
TTGGAGACATTGCATTCTACTACTGCTCTGATGGTTACAGCCTAGCAGACAATTCCCAGCTTCTCTGC
AATGCCCAGGGCAAGTGGGTACCCCCAGAAGGTCAAGACATGCCCCGTTGTATAGCTCATTTCTGTGA
AAAACCTCCATCGGTTTCCTATAGCATCTTGGAATCTGTGAGCAAAGCAAAATTTGCAGCTGGCTCAG
TTGTGAGCTTTAAATGCATGGAAGGCTTTGTACTGAACACCTCAGCAAAGATTGAATGTATGAGAGGT
GGGCAGTGGAACCCTTCCCCCATGTCCATCCAGTGCATCCCTGTGCGGTGTGGAGAGCCACCAAGCAT
CATGAATGGCTATGCAAGTGGATCAAACTACAGTTTTGGAGCCATGGTGGCTTACAGCTGCAACAAGG
GGTTCTACATCAAAGGGGAAAAGAAGAGCACCTGCGAAGCCACAGGGCAGTGGAGTAGTCCTATACCG
ACGTGCCACCCGGTATCTTGTGGTGAACCACCTAAGGTTGAGAATGGCTTTCTGGAGCATACAACTGG
CAGGATCTTTGAGAGTGAAGTGAGGTATCAGTGTAACCCGGGCTATAAGTCAGTCGGAAGTCCTGTAT
TTGTCTGCCAAGCCAATCGCCACTGGCACAGTGAATCCCCTCTGATGTGTGTTCCTCTCGACTGTGGA
AAACCTCCCCCGATCCAGAATGGCTTCATGAAAGGAGAAAACTTTGAAGTAGGGTCCAAGGTTCAGTT
TTTCTGTAATGAGGGTTATGAGCTTGTTGGTGACAGTTCTTGGACATGTCAGAAATCTGGCAAATGGA
ATAAGAAGTCAAATCCAAAGTGCATGCCTGCCAAGTGCCCAGAGCCGCCCCTCTTGGAAAACCAGCTA
GTATTAAAGGAGTTGACCACCGAGGTAGGAGTTGTGACATTTTCCTGTAAAGAAGGGCATGTCCTGCA
AGGCCCCTCTGTCCTGAAATGCTTGCCATCCCAGCAATGGAATGACTCTTTCCCTGTTTGTAAGATTG
TTCTTTGTGCCCCACCTCCCCTAATTTCCTTTGGTGTCCCCATTCCTTCTTCTGCTCTTCATTTTGGA
AGTACTGTCAAGTATTCTTGTGTAGGTGGGTTTTTCCTAAGAGGAAATTCTACCACCCTCTGCCAACC
TGATGGCACCTGGAGCTCTCCACTGCCAGAATGTGTTCCAGTAGAATGTCCCCAACCTGAGGAAATCC
CCAATGGAATCATTGATGTGCAAGGCCTTGCCTATCTCAGCACAGCTCTCTATACCTGCAAGCCAGGC
TTTGAATTGGTGGGAAATACTACCACCCTTTGTGGAGAAAATGGTCACTGGCTTGGAGGAAAACCAAC
ATGTAAAGCCATTGAGTGCCTGAAACCCAAGGAGATTTTGAATGGCAAATTCTCTTACACGGACCTAC
ACTATGGACAGACCGTTACCTACTCTTGCAACCGAGGCTTTCGGCTCGAAGGTCCCAGTGCCTTGACC
TGTTTAGAGACAGGTGATTGGGATGTAGATGCCCCATCTTGCAATGCCATCCACTGTGATTCCCCACA
ACCCATTGAAAATGGTTTTGTAGAAGGTGCAGATTACAGCTATGGTGCCATAATCATCTACAGTTGCT
TCCCTGGGTTTCAGGTGGCTGGTCATGCCATGCAGACCTGTGAAGAGTCAGGATGGTCAAGTTCCATC
CCAACATGTATGCCAATAGACTGTGGCCTCCCTCCTCATATAGATTTTGGAGACTGTACTAAACTCAA
AGATGACCAGGGATATTTTGAGCAAGAAGACGACATGATGGAAGTTCCATATGTGACTCCTCACCCTC
CTTATCATTTGGGAGCAGTGGCTAAAACCTGGGAAAATACAAAGGAGTCTCCTGCTACACATTCATCA
AACTTTCTGTATGGTACCATGGTTTCATACACCTGTAATCCAGGATATGAACTTCTGGGGAACCCTGT
GCTGATCTGCCAGGAAGATGGAACTTGGAATGGCAGTGCACCATCCTGCATTTCAATTGAATGTGACT
AGCTGTAAACCTGGACACATTCTAGCAGGCTCTGACTTAAGGCTTTGTCTAGAGAATAGAAAGTGGAG
TGGTGCCTCCCCACGCTGTGAAGCCATTTCATGCAAAAAGCCAAATCCAGTCATGAATGGATCCATCA
AAGGAAGCAACTACACATACCTGAGCACGTTGTACTATGAGTGTGACCCCGGATATGTGCTGAATGGC
ACTGAGAGGAGAACATGCCAGGATGACAAAAACTGGGATGAGGATGAGCCCATTTGCATTCCTGTGGA
CTGCAGTTCACCCCCAGTCTCAGCCAATGGCCAGGTGAGAGGAGACGAGTACACATTCCAAAAAGAGA
TTGAATACACTTGCAATGAAGGGTTCTTGCTTGAGGGAGCCAGGAGTCGGGTTTGTCTTGCCAATGGA
AGTTGGAGTGGAGCCACTCCCGACTGTGTGCCTGTCAGATGTGCCACCCCGCCACAACTGGCCAATGG
GGTGACGGAAGGCCTGGACTATGGCTTCATGAAGGAAGTAACATTCCACTGTCACGAGGGCTACATCT
TGCACGGTGCTCCAAAACTCACCTGTCAGTCAGATGGCAACTGGGATGCAGAGATTCCTCTCTGTAAA
CCAGTCAACTGTGGACCTCCTGAAGATCTTGCCCATGGTTTCCCTAATGGTTTTTCCTTTATTCATGG
GGGCCATATACAGTATCAGTGCTTTCCTGGTTATAAGCTCCATGGAAATTCATCAAGAAGGTGCCTCT
CCAATGGCTCCTGGAGTGGCAGCTCACCTTCCTGCCTGCCTTGCAGATGTTCCACACCAGTAATTGAA
TATGGAACTGTCAATGGGACAGATTTTGACTGTGGAAAGGCAGCCCGGATTCAGTGCTTCAAAGGCTT
CAAGCTCCTAGGACTTTCTGAAATCACCTGTGAAGCCGATGGCCAGTGGAGCTCTGGGTTCCCCCACT
GTGAACACACTTCTTGTGGTTCTCTTCCAATGATACCAAATGCGTTCATCAGTGAGACCAGCTCTTGG
AAGGAAAATGTGATAACTTACAGCTGCAGGTCTGGATATGTCATACAAGGCAGTTCAGATCTGATTTG
TACAGAGAAAGGGGTATGGAGCCAGCCTTATCCAGTCTGTGAGCCCTTGTCCTGTGGGTCCCCACCGT
CTGTCGCCAATGCAGTGGCAACTGGAGAGGCACACACCTATGAAAGTGAAGTGAAACTCAGATGTCTG
GAAGGTTATACGATGGATACAGATACAGATACATTCACCTGTCAGAAAGATGGTCGCTGGTTCCCTGA
GAGAATCTCCTGCAGTCCTAAAAAATGTCCTCTCCCGGAAAACATAACACATATACTTGTACATGGGG
ACGATTTCAGTGTGAATAGGCAAGTTTCTGTGTCATGTGCAGAAGGGTATACCTTTGAGGGAGTTAAC
ATATCAGTATGTCAGCTTGATGGAACCTGGGAGCCACCATTCTCCGATGAATCTTGCAGTCCAGTTTC
TTGTGGGAAACCTGAAAGTCCAGAAACATGGATTTGTGGTTGGCAGTAAATACACCTTTGAAAGCACA
TTATTTATCAGTGTGAGCCTGGCTATGAACTAGAGGGGAACAGGGAACGTGTCTGCCAGGAGAACAGA
CAGTGGAGTGGAGGGGTGGCAATATGCAAAGAGACCAGGTGTGAAACTCCACTTGAATTTCTCAATGG
GAAAGCTGACATTGAAAACAGGACGACTGGACCCAACGTGGTATATTCCTGCAACAGAGGCTACAGTC
TTGAAGGGCCATCTGAGGCACACTGCACAGAAAATGGAACCTGGAGCCACCCAGTCCCTCTCTGCAAA
CCAAATCCATGCCCTGTTCCTTTTGTGATTCCCGAGAATGCTCTGCTGTCTGAAAAGGAGTTTTATGT
TGATCAGAATGTGTCCATCAAATGTAGGGAAGGTTTTCTGCTGCAGGGCCACGGCATCATTACCTGCA
ACCCCGACGAGACGTGGACACAGACAAGCGCCAAATGTGAAAAAATCTCATGTGGTCCACCAGCTCAC
GTAGAAAATGCAATTGCTCGAGGCGTACATTATCAATATGGAGACATGATCACCTACTCATGTTACAG
TGGATACATGTTGGAGGGTTTCCTGAGGAGTGTTTGTTTAGAAAATGGAACATGGACATCACCTCCTA
TTTGCAGAGCTGTCTGTCGATTTCCATGTCAGAATGGGGGCATCTGCCAACGCCCAAATGCTTGTTCC
TGTCCAGAGGGCTGGATGGGGCGCCTCTGTGAAGAACCAATCTGCATTCTTCCCTGTCTGAACGGAGG
TCGCTGTGTGGCCCCTTACCAGTGTGACTGCCCGCCTGGCTGGACGGGGTCTCGCTGTCATACAGCTG
TTTGCCAGTCTCCCTGCTTAAATGGTGGAAAATGTGTAAGACCAAACCGATGTCACTGTCTTTCTTCT
TGGACGGGACATAACTGTTCCAGGAAAAGGAGGACTGGGTTTTAACCACTGCACGACCATCTGGCTCT
CCCAAAAGCAGGATCATCTCTCCTCGGTAGTGCCTGGGCATCCTGGAACTTATGCAAAGAAAGTCCAA
CATGGTGCTGGGTCTTGTTTAGTAAACTTGTTACTTGGGGTTACTTTTTTTATTTTGTGATATATTTT
GTTATTCCTTGTGACATACTTTCTTACATGTTTCCATTTTTAAATATGCCTGTATTTTCTATATAAAA
ATTATATTAAATAGATGCTGCTCTACCCTCACAAAATGTACATATTCTGCTGTCTATTGGGAAAGTTC
CTGGTACACATTTTTATTCAGTTACTTAAAATGATTTTTCCATTAAAGTATATTTTGCTACTAAATAA
AAAAAA

| | | |
|---|---|---|
| NOV8m, SNP13374257 of | SEQ ID NO: 202 | MW at 389982.5kD |

| | | | |
|---|---|---|---|
| CG50646-04, Protein Sequence | SNP Pos: 2381 | 3570 aa | SNP Change: Thr to Ala |

MRRICAACWGLALVSGWATFQQMSPSRNFSFRLFPETAPGAPGSIPAPPAPGDEAAGSRVERLGQAFR
VRLLRELSERLELVFLVDDSSSVGEVNFRSELMFVRKLLSDFPVVPTATRVAIVTFSSKNYVVPRVDY
ISTRRARQHKCALLLQEIPAISYRGGGTYTKGAFQQAAQILLHARENSTKVVFLITDGYSNGGDPRPI
AASLRDSGVEIFTFGIWQGNIRELNDMASTPKEEHCYLLHSFEEFEALVALCHMLFVDLPSGSFIQDD
MVHCSYLCDEGKDCCDRMGSCKCGKHTGHFECICEKGYNGKGLQYDCTVCPSGTYKPEGSPGGISSCI
PCPDENHTSPPGSTSPEDCVCREGYPASGQTCEVVHCPALKPPENGYFIQNTCNNHFNAACGVRCHPG
FDLVGSSIILCLPNGLWSGSESYCRVRTCPHLRQPKHGHISCSTREMLYKTTCLVACDEGYRLEGSDK
LTCQGNSQWDGPEPRCVERHCSTFQMPKDVIISPHNCGKQPAKFGTICYVSCRQGFILSGVKEMLRCT
TSGKWNVGVQAAVCKDVEAPQINCPKDIEAKTLEQQDSANVTWQIPTAKDNSGEKVSVHVHPAFTPPY
LFPIGDVAIVYTATDLSGNQASCIFHIKVIDAEPPVIDWCRSPPPVQVSEKVHAASWDEPQFSDNSGA
ELVITRSHTQGDLFPQGETIVQYTATDPSGNNRTCDIHIVIKGSPCEIPFTPVNGDFICTPDNTGVNC
TLTCLEGYDFTEGSTDKYYCAYEDGVWKPTYTTEWPDCASKRFANRGFKSFEMFYKAARCDDTDLMKK
FSEAFETTLGKMVPSFCSDAEDIDCPLEENLTKKYCLEYNYDYENGFAIGPGGWGAANRLDYSYDDFL
DTVQETATSIGNAKSSRIKRSAPLSDYKIKLIFNITASVPLPDERNDTLEWENQQRLLQTLETITNKL
KRTLNKDPMYSFQLASEILIADSNSLETKKASPFCRPGSVLRGRMCVNCPLGTYYNLEHFTCESCRIG
SYQDEEGQLECKLCPSGMYTEYIHSRNISDCKAQCKQGTYSYSGLETCESCPLGTYQPKFGSRSCLSC
TECSSFSSTFSAAEESVVPPASLGHIKKRHEISSQASHECFFNPCHNSGTCQQLGRGYVCLCPLGYTG
LKCETDIDECSPLPCLNNGVCKDLVGEFICECPSGYTGKHCELNINECQSNPCRNQATCVDELNSYSC
KCQPGFSGKRCETGMYQLSVINNLNNAVCEDQVGGFLCKCPPGFLGTRCGKNVDECLSQPCKNGATCK
DGANSFRCLCAAGFTGSHCELNINECQSNPCRNQATCVDELNSYSCKCQPGFSGKRCETEQSTGFNLD
FEVSGIYGYVMLDGMLPSLHALTCTFWMKSSDDMNYGTPISYAVDNGSDNTLLLTDYNGWVLYVNGRE
KITNCPSVNDGRWHHIAITWTSTGGAWRVYINGELSDGGTGLSIGKAIPGGGALVLGQEQDKKGEGFN
PAESFVGSISQLNLWDYVLSPQQVKSLATSCPEELSKGNVLAWPDFLSGIVGKVKIDSKSIFCSDCPR
LGGSVPHLRTASEDLKPGSKVNLFCEPGFQLVGNPVQYCLNQGQWTQPLPHCERIRCGVPPPLENGFH
SADDFYAGSTVTYQCNNGYYLLGDSRMFCTDNGSWNGVSPSCLDVDECAVGSDCSEHASCLNVDGSYI
CSCVPPYTGDGKNCAEPIKCKAPGNPENGHSSGEIYTVGAEVTFSCQEGYQLMGVTKITCLESGEWNH
LIPYCKAVSCGKPAIPENGCIEELAFTFGSKVTYRCNKGYTLAGDKESSCLANSSWSHSPPVCEPVKC
SSPENINNGKYILSGLTYLSTASYSCDTGYSLQGPSIIECTASGIWDRAPPACHLVFCGEPPAIKDAV
ITGNNFTFRNTVTYTCKEGYTLAGLDTIECLADGKWSRSDQQCLAVSCDEPPIVDHASPETAHRLFGD
IAFYYCSDGYSLADNSQLLCNAQGKWVPPEGQDMPRCIAHFCEKPPSVSYSILESVSKAKFAAGSVVS
FKCMEGFVLNTSAKIECMRGGQWNPSPMSIQCIPVRCGEPPSIMNGYASGSNYSFGAMVAYSCNKGFY
IKGEKKSTCEATGQWSSPIPTCHPVSCGEPPKVENGFLEHTTGRIFESEVRYQCNPGYKSVGSPVFVC
QANRHWHSESPLMCVPLDCGKPPPIQNGFMKGENFEVGSKVQFFCNEGYELVGDSSWTCQKSGKWNKK
SNPKCMPAKCPEPPLLENQLVLKELTTEVGVVTFSCKEGHVLQGPSVLKCLPSQQWNDSFPVCKIVLC
APPPLISFGVPIPSSALHFGSTVKYSCVGGFFLRGNSTTLCQPDGTWSSPLPECVPVECPQPEEIPNG
IIDVQGLAYLSTALYTCKPGFELVGNTTTLCGENGHWLGGKPTCKAIECLKPKEILNGKFSYTDLHYG
QTVTYSCNRGFRLEGPSALTCLETGDWDVDAPSCNAIHCDSPQPIENGFVEGADYSYGAIIIYSCFPG
FQVAGHAMQTCEESGWSSSIPTCMPIDCGLPPHIDFGDCTKLKDDQGYFEQEDDNMEVPYVTPHPPYH
LGAVAKTWENTKESPATHSSNFLYGTMVSYTCNPGYELLGNPVLICQEDGTWNGSAPSCISIECDLPT
APENGFLRFTETSMGSAVQYSCKPGHILAGSDLRLCLENRKWSGASPRCEAISCKKPNPVMNGSIKGS
NYTYLSTLYYECDPGYVLNGTERRTCQDDKNWDEDEPICIPVDCSSPPVSANGQVRGDEYTFQKEIEY
TCNEGFLLEGARSRVCLANGSWSGATPDCVPVRCATPPQLANGVTEGLDYGFMKEVTFHCHEGYILHG
APKLTCQSDGNWDAEIPLCKPVNCGPPEDLAHGFPNGFSFIHGGHIQYQCFPGYKLHGNSSRRCLSNG
SWSGSSPSCLPCRCSTPVIEYGTVNGTDFDCGKAARIQCFKGFKLLGLSEITCEADGQWSSGFPHCEH
TSCGSLPMIPNAFISETSSWKENVITYSCRSGYVIQGSSDLICTEKGVWSQPYPVCEPLSCGSPPSVA
NAVATGEAHTYESEVKLRCLEGYTMDTDTDTFTCQKDGRWFPERISCSPKKCPLPENITHILVHGDDF
SVNRQVSVSCAEGYTFEGVNISVCQLDGTWEPPFSDESCSPVSCGKFESPEHGFVVGSKYTFESTIIY
QCEPGYELEGNRERVCQENRQWSGGVAICKETRCETPLEFLNGKADIENRTTGPNVVYSCNRGYSLEG
PSEAHCTENGTWSHPVPLCKPNPCPVPFVIPENALLSEKEFYVDQNVSIKCREGFLLQGHGIITCNPD
ETWTQTSAKCEKISCGPPAHVENAIARGVHYGYGDMITYSCYSGYMLEGFLRSVCLENGTWTSPPICR
AVCRFPCQNGGICQRPNACSCPEGWMGRLCEEPICILPCLNGGRCVAPYQCDCPPGWTGSRCHTAVCQ
SPCLNGGKCVRPNRCHCLSSWTGHNCSRKRRTGF

| | |
|---|---|
| SEQ ID NO: 203 | 11158 bp |

| | | |
|---|---|---|
| NOV8n, SNP13382479 of | ORF Start: ATG at 77 | ORF Stop: TAA at 10787 |

| | | |
|---|---|---|
| CG50646-04, DNA Sequence | SNP Pos: 9649 | SNP Change: A to T |

CAATTGGTCTAGGGTCTCCCCCATTGGAATATCCATCAGTGATGAGAAATACAACGTTTGTTGAGTTT
TCTCTAGCATGAGAAGAATTTGCGCGGCTTGCTGGGGTCTGGCGCTCGTTTCGGGCTGGGCGACCTTT
CAGCAGATGTCCCCGTCGCGCAATTTCAGCTTCCGCCTCTTCCCCGAGACCCCGCCCGGGGCCCCCCG
CGTTCCGCGTGCGGCTGCTGCGGGAGCTCAGCGAGCGCCTGGAGCTTGTCTTCCTGGTGGATGATTCG
TCCAGCGTGGGCGAAGTCAACTTCCGCAGCGAGCTCATGTTCGTCCGCAAGCTGCTGTCCGACTTCCC
TCGATTACATCTCCACCCGCCGCGCGCGCCAGCACAAGTGCGCGCTGCTCCTCCAAGAGATCCCTGCC
ATCTCCTACCGAGGTGGCGGCACCTACACCAAGGGCGCCTTCCAGCAAGCCGCGCAAATTCTTCTTCA
TGCTAGAGAAAACTCAACAAAAGTTGTATTTCTCATCACTGATGGATATTCCAATGGGGGAGACCCTA
GACCAATTGCAGCGTCACTGCGAGATTCAGGAGTGGAGATCTTCACTTTTGGCATATGGCAAGGGAAC
ATTCGAGAGCTGAATGACATGGCTTCCACCCCAAAGGAGGAGCACTGTTACCTGCTACACAGTTTTGA
AGAATTTGAGGCTTTAGTCGCCCTCTGTCATATGTTATTTGTAGATCTACCTTCTGGGAGTTTTATTC
AAGATGATATGGTCCACTGCTCATATCTTTGTGATGAAGGCAAGGACTGCTGTGACCGAATGGGAAGC
TGCAAATGTGGGAAACACACAGGCCATTTTGAGTGCATCTGTGAAAAGGGGTATAACGGGAAAGGTCT
GCAGTATGACTGCACAGTTTGCCCATCGGGGACATACAAACCTGAAGGCTCACCAGGAGGAATCAGCA
GTTGCATTCCATGTCCTGATGAAAATCACACCTCTCCACCTGGAAGCACATCCCCTGAAGACTGTGTC
TGCAGAGAGGGATACAGGGCATCTGGCCAGACCTGTGAAGTTGTCCACTGCCCTGCCCTGAAGCCTCC
CGAAAATGGTTACTTTATCCAAAACACTTGCAACAACCACTTCAATGCAGCCTGTGGGGTCCGATGTC
ACCCTGGATTTGATCTTGTGGGAAGCAGCATCATCTTATGTCTACCCAATGGTTTGTGGTCCGGTTCA
GAGAGCTACTGCAGAGTAAGAACATGTCCTCATCTCCGCCAGCCGAAACATGGCCACATCAGCTGTTC
TACAAGGGAAATGTTATATAAGACAACATGTTTGGTTGCCTGTGATGAAGGGTACAGGCTAGAAGGCA
GTGATAAGCTTACTTGTCAAGGAAACAGCCAGTGGGATGGGCCAGAACCCCGGTGTGTGGAGCGCCAC
TGTTCCACCTTTCAGATGCCCAAAGATGTCATCATATCCCCCCACAACTGTGGCAAGCAGCCAGCCAA
ATTTGGGACGATCTGCTATGTAAGTTGCCGCCAAGGGTTCATTTTATCTGGAGTCAAAGAAATGCTGA
GATGTACCACTTCTGGAAAATGGAATGTCGGAGTTCAGGCAGCTGTGTGTAAAGACGTGGAGGCTCCT
CAAATCAACTGTCCTAAGGACATAGAGGCTAAGACTCTGGAACAGCAAGATTCTGCCAATGTTACCTG
GCAGATTCCAACAGCTAAAGACAACTCTGGTGAAAAGGTGTCAGTCCACGTTCATCCAGCTTTCACCC
CACCTTACCTTTTCCCAATTGGAGATGTTGCTATCGTATACACGGCAACTGACCTATCCGGCAACCAG
GCCAGCTGCATTTTCCATATCAAGGTTATTGATGCAGAACCACCTGTCATAGACTGGTGCAGATCTCC
ACCTCCCGTCCAGGTCTCGGAGAAGGTACATGCCGCAAGCTGGGATGAGCCTCAGTTCTCAGACAACT
CAGGTGCTGAATTGGTCATTACCAGAAGTCATACACAAGGAGACCTTTTCCCTCAAGGGGAGACTATA
GTACAGTATACAGCCACTGACCCCTCAGGCAATAACAGGACATGTGATATCCATATTGTCATAAAAGG
TTCTCCCTGTGAAATTCCATTCACACCTGTAAATGGGGATTTTATATGCACTCCAGATAATACTGGAG
TCAACTGTACATTAACTTGCTTGGAGGGCTATGATTTCACAGAAGGGTCTACTGACAAGTATTATTGT
GCTTATGAAGATGGCGTCTGGAAACCAACATATACCACTGAATGGCCAGACTGTGCCAGTAAGCGTTT
TGCAAACCACGGGTTCAAGTCCTTTGAGATGTTCTACAAAGCAGCTCGTTGTGATGACACAGATCTGA
TGAAGAAGTTTTCTGAAGCATTTGAGACGACCCTGGGAAAAATGGTCCCATCATTTTGTAGTGATGCA
GAGGACATTGACTGCAGACTGGAGGAGAACCTGACCAAAAAATATTGCCTAGAATATAATTATGACTA
TGAAAATGGCTTTGCAATTGGTCCAGGTGGCTGGGGTGCAGCTAATAGGCTGGATTACTCTTACGATG
ACTTCCTGGACACTGTGCAAGAAACAGCCACAAGCATCGGCAATGCCAAGTCCTCACGGATTAAAAGA
AGTGCCCCATTATCTGACTATAAAATTAAGTTAATTTTTAACATCACAGCTAGTGTGCCATTACCCGA
TGAAAGAAATGATACCCTTGAATGGGAAAATCAGCAACGACTCCTTCAGACATTGGAAACTATCACAA
ATAAACTGAAAAGGACTCTCAACAAAGACCCCATGTATTCCTTTCAGCTTGCATCAGAAATACTTATA
GCCGACAGCAATTCATTAGAAACAAAAAAGGCTTCCCCCTTCTGCAGACCAGGCTCAGTGCTGAGAGG
GCGTATGTGTGTCAATTGCCCTTTGGGAACCTATTATAATCTGGAACATTTCACCTGTGAAAGCTGCC
GGATCGGATCCTATCAAGATGAAGAAGGGCAACTTGAGTGCAAGCTTTGCCCCTCTGGGATGTACACG
GAATATATCCATTCAAGAAACATCTCTGATTGTAAAGCTCAGTGTAAACAAGGCACCTACTCATACAG
TGGACTTGAGACTTGTGAATCGTGTCCACTGGGCACTTATCAGCCAAAATTTGGTTCCCGGAGCTGCC
TCTCGTGTCCAGAAAACACCTCAACTGTGAAAAGAGGAGCCGTGAACATTTCTGCATGTGGAGTTCCT
TGTCCAGAAGGAAAATTCTCGCGTTCTGGGTTAATGCCCTGTCACCCATGTCCTCGTGACTATTACCA
ACCTAATGCAGGGAAGGCCTTCTGCCTGGCCTGTCCCTTTTATGGAACTACCCCATTCGCTGGTTCCA
GATCCATCACAGAATGTTCAAGTTTTAGTTCAACTTTCTCAGCGGCAGAGGAAAGTGTGGTGCCCCCT
GCCTCTCTTGGACATATTAAAAAGAGGCATGAAATCAGCAGTCAGGCAAGTCATGAATGCTTCTTTAA
CCCTTGCCACAATAGTGGAACCTGCCAGCAACTTGGGCGTGGTTATGTTTGTCTCTGTCCACTTGGAT
ATACAGGTTTAAAGTGTGAAACAGACATCGATGAGTGCAGCCCACTGCCTTGCCTCAACAATGGAGTT
TGTAAGACCTAGTTGGGGAAATTCATTTGTGAGTGCCCATCAGGTTACACAGGTAAGCACTGTGAATT
GAACATCAATGAATGTCAGTCTAATCCATGTAGAAATCAGGCCACCTGTGTGGATGAATTAAATTCAT
ACAGTTGTAAATGTCAGCCAGGATTTTCAGGCAAAAGGTGTGAAACAGGTATGTATCAACTCAGTGTT
ATTAATAACCTTAATAATGCAGTCTGTGAAGACCAGGTTGGGGGATTCTTGTGCAAATGCCCACCTGG
ATTTTTGGGTACCCGATGTGGAAAGAACGTCGATGAGTGTCTCAGTCAGCCATGCAAAAATGGAGCTA
CCTGTAAAGACGGTGCCAATAGCTTCAGGTGCCTGTGTGCAGCTGGCTTCACAGGATCACACTGTGAA
TTGAACATCAATGAATGTCAGTCTAATCCATGTAGAAATCAGGCCACCTGTGTGGATGAATTAAATTC
ATACAGTTGTAAATGTCAGCCAGGATTTTCAGGCAAAAGGTGTGAAACAGAACAGTCTACAGGCTTTA
ACCTGGATTTTGAAGTTTCTGGCATCTATGGATATGTCATGCTAGATGGCATGCTCCCATCTCTCCAT
GCTCTAACCTGTACCTTCTGGATGAAATCCTCTGACGACATGAACTATGGAACACCAATCTCCTATGC
AGTTGATAACGGCAGCGACAATACCTTGCTCCTGACTGATTATAACGGGTGGGTTCTTTATGTGAATG
GCAGGGAAAAGATAACAAACTGTCCCTCGGTGAATGATGGCAGATGGCATCATATTGCAATCACTTGG
ACAAGTACTGGTGGAGCCTGGAGGGTCTATATAAATGGGGAATTATCTGACGGTGGTACTGGCCTCTC
CATTGGCAAAGCCATACCTGGTGGCGGTGCATTAGTTCTTGGGCAAGAGCAAGACAAAAAAGGAGAGG
GGTTCAACCCGGCTGAGTCTTTTGTGGGCTCCATAAGCCAGCTCAACCTCTGGGACTATGTCCTGTCT
CCACAGCAGGTGAAGTCACTGGCTACCTCCTGCCCAGAGGAACTCAGTAAAGGAAACGTGTTAGCATG
GCCTGATTTCTTGTCAGGAATTGTGGGGAAAGTGAAGATCGATTCTAAGAGCATATTTTGTTCTGATT
GCCCACGCTTGGGAGGGTCAGTGCCTCATCTGAGAACTGCATCTGAAGATTTAAAACCAGGTTCCAAA
ACAGTGGACACAACCACTCCCCCACTGTGAACGCATTCGCTGTGGGGTGCCACCTCCTTTGGAGAATG
GCTTCCATTCAGCCGATGACTTCTATGCTGGCAGCACAGTAACCTACCAGTGCAACAATGGCTACTAT
CTATTGGGTGACTCAAGGATGTTCTGTACAGATAATGGGAGCTGGAACGGCGTTTCACCATCCTGCTT
AGATGTCGATGAGTGTGCAGTTGGATCAGATTGTAGTGAGCATGCTTCTTGCCTGAACGTAGATGGAT
CCTACATATGTTCATGTGTCCCACCGTACACAGGAGATGGGAAAAACTGTGCAGAACCTATAAAATGT
AAGGCTCCAGGAAATCCGGAAAATGGCCACTCCTCAGGTGAGATTTATACAGTAGGTGCCGAAGTCAC
ATTTTCGTGTCAGGAAGGATACCAGTTGATGGGAGTAACCAAAATCACATGTTTGGAGTCTGGAGAAT
GGAATCATCTAATACCATATTGTAAAGCTGTTTCATGTGGTAAACCGGCTATTCCAGAAAATGGTTGC
ATTGAGGAGTTAGCATTTACTTTTGGCAGCAAAGTGACATATAGGTGTAATAAAGGATATACTCTGGC
CGGTGATAAAGAATCATCCTGTCTTGCTAACAGTTCTTGGAGTCATTCCCCTCCTGTGTGTGAACCAG
TGAAGTGTTCTAGTCCGGAAAATATAAATAATGGAAAATATATTTTGAGTGGGCTTACCTACCTTTCT
ACTGCATCATATTCATGCGATACAGGATACAGCTTACAGGGCCCTTCCATTATTGAATGCACGGCTTC
TGGCATCTGGGACAGAGCGCCACCTGCCTGTCACCTCGTCTTCTGTGGAGAACCACCTGCCATCAAAG
ATGCTGTCATTACGGGGAATAACTTCACTTTCAGGAACACCGTCACTTACACTTGCAAAGAAGGCTAT
ACTCTTGCTGGTCTTGACACCATTGAATGCCTGGCCGACGGCAAGTGGAGTAGAAGTGACCAGCAGTG
CCTGGCTGTCTCCTGTGATGAGCCACCCATTGTGGACCACGCCTCTCCAGAGACTGCCCATCGGCTCT
AATGCCCAGGGCAAGTGGGTACCCCCAGAAGGTCAAGACATGCCCCGTTGTATAGCTCATTTCTGTGA
AAAACCTCCATCGGTTTCCTATAGCATCTTGGAATCTGTGAGCAAAGCAAAATTTGCAGCTGGCTCAG
TTGTGAGCTTTAAATGCATGGAAGGCTTTGTACTGAACACCTCAGCAAAGATTGAATGTATGAGAGGT
GGGCAGTGGAACCCTTCCCCCATGTCCATCCAGTGCATCCCTGTGCGGTGTGGAGAGCCACCAAGCAT
CATGAATGGCTATGCAAGTGGATCAAACTACAGTTTTGGAGCCATGGTGGCTTACAGCTGCAACAAGG
GGTTCTACATCAAAGGGGAAAAGAAGAGCACCTGCGAAGCCACAGGGCAGTGGAGTAGTCCTATACCG
ACGTGCCACCCGGTATCTTGTGGTGAACCACCTAAGGTTGAGAATGGCTTTCTGGAGCATACAACTGG
CAGGATCTTTGAGAGTGAAGTGAGGTATCAGTGTAACCCGGGCTATAAGTCAGTCGGAAGTCCTGTAT
TTGTCTGCCAAGCCAATCGCCACTGGCACAGTGAATCCCCTCTGATGTGTGTTCCTCTCGACTGTGGA
AAACCTCCCCCGATCCAGAATGGCTTCATGAAAGGAGAAAACTTTGAAGTAGGGTCCAAGGTTCAGTT
TTTCTGTAATGAGGGTTATGAGCTTGTTGGTGACAGTTCTTGGACATGTCAGAAATCTGGCAAATGGA
ATAAGAAGTCAAATCCAAAGTGCATGCCTGCCAAGTGCCCAGAGCCGCCCCTCTTGGAAAACCAGCTA
GTATTAAAGGAGTTGACCACCGAGGTAGGAGTTGTGACATTTTCCTGTAAAGAAGGGCATGTCCTGCA
AGGCCCCTCTGTCCTGAAATGCTTGCCATCCCAGCAATGGAATGACTCTTTCCCTGTTTGTAAGATTG
TTCTTTGTACCCCACCTCCCCTAATTTCCTTTGGTGTCCCCATTCCTTCTTCTGCTCTTCATTTTGGA
AGTACTGTCAAGTATTCTTGTGTAGGTGGGTTTTTCCTAAGAGGAAATTCTACCACCCTCTGCCAACC
TGATGGCACCTGGAGCTCTCCACTGCCAGAATGTGTTCCAGTAGAATGTCCCCAACCTGAGGAAATCC
CCAATGGAATCATTGATGTGCAAGGCCTTGCCTATCTCAGCACAGCTCTCTATACCTGCAAGCCAGGC
TTTGAATTGGTGGGAAATACTACCACCCTTTGTGGAGAAAATGGTCACTGGCTTGGAGGAAAACCAAC
ATGTAAAGCCATTGAGTGCCTGAAACCCAAGGAGATTTTGAATGGCAAATTCTCTTACACGGACCTAC
ACTATGGACAGACCGTTACCTACTCTTGCAACCGAGGCTTTCGGCTCGAAGGTCCCAGTGCCTTGACC
TGTTTAGAGACAGGTGATTGGGATGTAGATGCCCCATCTTGCAATGCCATCCACTGTGATTCCCCACA
ACCCATTGAAAATGGTTTTGTAGAAGGTGCAGATTACAGCTATGGTGCCATAATCATCTACAGTTGCT
TCCCTGGGTTTCAGGTGGCTGGTCATGCCATGCAGACCTGTGAAGAGTCAGGATGGTCAAGTTCCATC
CCAACATGTATGCCAATAGACTGTGGCCTCCCTCCTCATATAGATTTTGGAGACTGTACTAAACTCAA
AGATGACCAGGGATATTTTGAGCAAGAAGACGACATGATGGAAGTTCCATATGTGACTCCTCACCCTC
CTTATCATTTGGGAGCAGTGGCTAAAACCTGGGAAAATACAAAGGAGTCTCCTGCTACACATTCATCA
AACTTTCTGTATGGTACCATGGTTTCATACACCTGTAATCCAGGATATGAACTTCTGGGGAACCCTGT
GCTGATCTGCCAGGAAGATGGAACTTGGAATGGCAGTGCACCATCCTGCATTTCAATTGAATGTGACT
TGCCTACTGCTCCTGAAAATGGCTTTTTGCGTTTTACAGAGACTAGCATGGGAAGTGCTGTGCAGTAT
AGCTGTAAACCTGGACACATTCTAGCAGGCTCTGACTTAAGGCTTTGTCTAGAGAATAGAAAGTGGAG
TGGTGCCTCCCCACGCTGTGAAGCCATTTCATGCAAAAAGCCAAATCCAGTCATGAATGGATCCATCA
AAGGAAGCAACTACACATACCTGAGCACGTTGTACTATGAGTGTGACCCCGGATATGTGCTGAATGGC
ACTGAGAGGAGAACATGCCAGGATGACAAAAACTGGGATGAGGATGAGCCCATTTGCATTCCTGTGGA
CTGCAGTTCACCCCCAGTCTCAGCCAATGGCCAGGTGAGAGGAGACGAGTACACATTCCAAAAAGAGA
TTGAATACACTTGCAATGAAGGGTTCTTGCTTGAGGGAGCCAGGAGTCGGGTTTGTCTTGCCAATGGA
AGTTGGAGTGGAGCCACTCCCGACTGTGTGCCTGTCAGATGTGCCACCCCGCCACAACTGGCCAATGG
GGTGACGGAAGGCCTGGACTATGGCTTCATGAAGGAAGTAACATTCCACTGTCACGAGGGCTACATCT
TGCACGGTGCTCCAAAACTCACCTGTCAGTCAGATGGCAACTGGGATGCAGAGATTCCTCTCTGTAAA
CCAGTCAACTGTGGACCTCCTGAAGATCTTGCCCATGGTTTCCCTAATGGTTTTTCCTTTATTCATGG
GGGCCATATACAGTATCAGTGCTTTCCTGGTTATAAGCTCCATGGAAATTCATCAAGAAGGTGCCTCT
CCAATGGCTCCTGGAGTGGCAGCTCACCTTCCTGCCTGCCTTGCAGATGTTCCACACCAGTAATTGAA
TATGGAACTGTCAATGGGACAGATTTTGACTGTGGAAAGGCAGCCCGGATTCAGTGCTTCAAAGGCTT
CAAGCTCCTAGGACTTTCTGAAATCACCTGTGAAGCCGATGGCCAGTGGAGCTCTGGGTTCCCCCACT
GTGAACACACTTCTTGTGGTTCTCTTCCAATGATACCAAATGCGTTCATCAGTGAGACCAGCTCTTGG
AAGGAAAATGTGATAACTTACAGCTGCAGGTCTGGATATGTCATACAAGGCAGTTCAGATCTGATTTG
TACAGAGAAAGGGGTATGGAGCCAGCCTTATCCAGTCTGTGAGCCCTTGTCCTGTGGGTCCCCACCGT
CTGTCGCCAATGCAGTGGCAACTGGAGAGGCACACACCTATGAAAGTGAAGTGAAACTCAGATGTCTG
GAAGGTTATACGATGGATACAGATACAGATACATTCACCTGTCAGAAAGATGGTCGCTGGTTCCCTGA
GAGAATCTCCTGCAGTCCTAAAAAATGTCCTCTCCCGGAAAACATAACACATATACTTGTTCATGGGG
ACGATTTCAGTGTGAATAGGCAAGTTTCTGTGTCATGTGCAGAAGGGTATACCTTTGAGGGAGTTAAC
ATATCAGTATGTCAGCTTGATGGAACCTGGGAGCCACCATTCTCCGATGAATCTTGCAGTCCAGTTTC
TTGTGGGAAACCTGAAAGTCCAGAACATGGATTTGTGGTTGGCAGTAAATACACCTTTGAAAGCACAA
TTATTTATCAGTGTGAGCCTGGCTATGAACTAGAGGGGAACAGGGAACGTGTCTGCCAGGAGAACAGA
CAGTGGAGTGGAGGGGTGGCAATATGCAAAGAGACCAGGTGTGAAACTCCACTTGAATTTCTCAATGG
GAAAGCTGACATTGAAAACAGGACGACTGGACCCAACGTGGTATATTCCTGCAACAGAGGCTACAGTC
TTGAAGGGCCATCTGAGGCACACTGCACAGAAAATGGAACCTGGAGCCACCCAGTCCCTCTCTGCAAA
CCAAATCCATGCCCTGTTCCTTTTGTGATTCCCGAGAATGCTCTGCTGTCTGAAAAGGAGTTTTATGT
TGATCAGAATGTGTCCATCAAATGTAGGGAAGGTTTTCTGCTGCAGGGCCACGGCATCATTACCTGCA
ACCCCGACGAGACGTGGACACAGACAAGCGCCAAATGTGAAAAAATCTCATGTGGTCCACCAGCTCAC
GTAGAAAATGCAATTGCTCGAGGCGTACATTATCAATATGGAGACATGATCACCTACTCATGTTACAG
TGGATACATGTTGGAGGGTTTCCTGAGGAGTGTTTGTTTAGAAAATGGAACATGGACATCACCTCCTA
TTTGCAGAGCTGTCTGTCGATTTCCATGTCAGAATGGGGGCATCTGCCAACGCCCAAATGCTTGTTCC
TGTCCAGAGGGCTGGATGGGGCGCCTCTGTGAAGAACCAATCTGCATTCTTCCCTGTCTGAACGGAGG
TCGCTGTGTGGCCCCTTACCAGTGTGACTGCCCGCCTGGCTGGACGGGGTCTCGCTGTCATACAGCTG
TTTGCCAGTCTCCCTGCTTAAATGGTGGAAAATGTGTAAGACCAAACCGATGTCACTGTCTTTCTTCT
TGGACGGGACATAACTGTTCCAGGAAAAGGAGGACTGGGTTTTAACCACTGCACGACCATCTGGCTCT
CCCAAAAGCAGGATCATCTCTCCTCGGTAGTGCCTGGGCATCCTGGAACTTATGCAAAGAAAGTCCAA
CATGGTGCTGGGTCTTGTTTAGTAAACTTGTTACTTGGGGTTACTTTTTTTATTTTGTGATATATTTT
GTTATTCCTTGTGACATACTTTCTTACATGTTTCCATTTTTAAATATGCCTGTATTTTCTATATAAAA
ATTATATTAAATAGATGCTGCTCTACCCTCACAAAATGTACATATTCTGCTGTCTATTGGGAAAGTTC
CTGGTACACATTTTTATTCAGTTACTTAAAATGATTTTTCCATTAAAGTATATTTTGCTACTAAATAA
AAAAAA

| | | |
|---|---|---|
| NOV8n, SNP13382479 of | SEQ ID NO: 204 | MW at 390012.5kD |

| | | | |
|---|---|---|---|
| CG50646-04, Protein Sequence | SNP Pos: 3191 | 3570 aa | SNP Change: Val to Val |

MRRICAACWGLALVSGWATFQQMSPSRNFSFRLFPETAPGAPGSIPAPPAPGDEAAGSRVERLGQAFR
VRLLRELSERLELVFLVDDSSSVGEVNFRSELMFVRKLLSDFPVVPTATRVAIVTFSSKNYVVPRVDY
ISTRRARQHKCALLLQEIPAISYRGGGTYTKGAFQQAAQILLHARENSTKVVFLITDGYSNGGDPRPI
AASLRDSGVEIFTFGIWQGNIRELNDMASTPKEEHCYLLHSFEEFEALVALCHMLFVDLPSGSFIQDD
MVHCSYLCDEGKDCCDRMGSCKCGKHTGHFECICEKGYNGKGLQYDCTVCPSGTYKPEGSPGGISSCI
PCPDENHTSPPGSTSPEDCVCREGYFASGQTCEVVHCPALKPPENGYFIQNTCNNHFNAACGVRCHPG
FDLVGSSIILCLPNGLWSGSESYCRVRTCPHLRQPKHGHISCSTREMLYKTTCLVACDEGYPLEGSDK
LTCQGNSQWDGPEPRCVERHCSTFQMPKDVIISPHNCGKQPAKFGTICYVSCRQGFILSGVKEMLRCT
TSGWNNVGVQAAVCKDVEAPQINCPKDIEAKTLEQQDSANVTWQIPTAKDNSGEKVSVHVHPAFTPPY
LFPIGDVAIVYTATDLSGNQASCIFHIKVIDAEPPVIDWCRSPPPVQVSEKVHAASWDEPQFSDNSGA
ELVITRSHTQGDLFPQGETIVQYTATDPSGNNRTCDIHIVIKGSPCEIPFTPVNGDFICTPDNTGVNC
TLTCLEGYDFTEGSTDKYYCAYEDGVWKPTYTTEWPDCASKRFANHGFKSFEMFYKAARCDDTDLMKK
FSEAFETTLGKMVPSFCSDAEDIDCRLEENLTKKYCLEYNYDYENGFAIGPGGWGAANRLDYSFDDFL
DTVQETATSIGNAKSSRIKRSAPLSDYKIKLIFNITASVPLPDERNDTLEWENQQRLLQTLETITNKL
KRTLNKDPMYSFQLASEILIADSNSLETKKASPFCRPGSVLRGRMCVNCPLGTYYNLEHFTCESCRIG
SYQDEEGQLECKLCPSGMYTEYIHSRNISDCKAQCKQGTYSYSGLETCESCPLGTYQPKFGSRSCLSC
PENTSTVKRGAVNISACGVPCPEGKFSRSGLMPCHPCPRDYYQPNAGKAFCLACPFYGTTPFAGSRSI
TECSSFSSTFSAAEESVVPPASLGHIKKRHEISSQASHECFFNPCHNSGTCQQLGRGYVCLCPLGYTG
LKCETDIDECSPLPCLNNGVCKDLVGEFICECPSGYTGKHCELNINECQSNPCRNQATCVDELNSYSC
KCQPGFSGKRCETGMYQLSVINNLNNAVCEDQVGGFLCKCPPGFLGTRCGKNVDECLSQPCKNGATCK
DGANSFRCLCAAGFTGSHCELNINECQSNPCRNQATCVDELNSYSCKCQPGFSGKRCETEQSTGFNLD
FEVSGIYGYVMLDGMLPSLHAITCTFWMKSSDDMNYGTPISYAVDNGSDNTLLLTDYNGWVLYVNGRE
KITNCPSVNDGRWHHIAITWTSTGGAWRVYINGELSDGGTGLSIGKAIPGGGALVLGQEQDKKGEGFN
PAESFVGSISQLNLWDYVLSPQQVKSLATSCPEELSKGNVLAWPDFLSGIVGKVKIDSKSIFCSDCPR
LGGSVPHLRTASEDLKPGSKVNLFCEPGFQLVGNPVQYCLNQGQWTQPLPHCERIRCGVPPPLENGFH
SADDFYAGSTVTYQCNNGYYLLGDSRMFCTDNGSWNGVSPSCLDVDECAVGSDCSEHASCLNVDGSYI
CSCVPPYTGDGKNCAEPIKCKAPGNPENGHSSGEIYTVGAEVTFSCQEGYQLMGVTKITCLESGEWNH
LIPYCKAVSCGKPAIPENGCIEELAFTFGSKVTYRCNKGYTLAGDKESSCLANSSWSHSPPVCEPVKC
SSPENINNGKYILSGLTYLSTASYSCDTGYSLQGPSIIECTASGIWDRAPPACHLVFCGEPPAIKDAV
ITGNNFTFRNTVTYTCKEGYTLAGLDTIECLADGKWSRSDQQCLAVSCDEPPIVDHASPETAHRLFGD
IAFYYCSDGYSLADNSQLLCNAQGKWVPPEGQDMPRCIAHFCEKPPSVSYSILESVSKAKFAAGSVVS
FKCMEGFVLNTSAKIECMRGGQWNPSPMSIQCIPVRCGEPPSIMNGYASGSNYSFGAMVAYSCNKGFY
IKGEKKSTCEATGQWSSPIPTCHPVSCGEPPKVENGFLEHTTGRIFESEVRYQCNPGYKSVGSPVFVC
QANRHWHSESPLMCVPLDCGKPPPIQNGFMKGENFEVGSKVQFFCNEGYELVGDSSWTCQKSGKWNKK
SNPKCMPAKCPEPPLLENQLVLKELTTEVGVVTFSCKEGHVLQGPSVLKCLPSQQWNDSFPVCKIVLC
TPPPLISFGVPIPSSALHFGSTVKYSCVGGFFLRGNSTTLCQPDGTWSSPLPECVPVECPQPEEIPNG
IIDVQGLAYLSTALYTCKPGFELVGNTTTLCGENGHWLGGKPTCKAIECLKPKEILNGKFSYTDLHYG
QTVTYSCNRGFRLEGPSALTCLETGDWDVDAPSCNAIHCDSPQPIENGFVEGADYSYGAIIIYSCFPG
FQVAGHAMQTCEESGWSSSIPTCMPIDCGLPPHIDFGDCTKLKDDQGYFEQEDDMMEVPYVTPHPPYH
LGAVAKTWENTKESPATHSSNFLYGTMVSYTCNPGYELLGNPVLICQEDGTWNGSAPSCISIECDLPT
APENGFLRFTETSMGSAVQYSCKPGHILAGSDLRLCLENRKWSGASPRCEAISCKKPNPVMNGSIKGS
NYTYLSTLYYECDPGYVLNGTERRTCQDDKNWDEDEPICIPVDCSSPPVSANGQVRGDEYTFQKEIEY
TCNEGFLLEGARSRVCLANGSWSGATPDCVPVRCATPPQLANGVTEGLDYGFMKEVTFHCHEGYILHG
APKLTCQSDGNWDAEIPLCKPVNCGPPEDLAHGFPNGFSFIHGGHIQYQCFPGYKLHGNSSRRCLSNG
SWSGSSPSCLPCRCSTPVIEYGTVNGTDFDCGKAARIQCFKGFKLLGLSEITCEADGQWSSGFPHCEH
TSCGSLPMIPNAFISETSSWKENVITYSCRSGYVIQGSSDLICTEKGVWSQPYPVCEPLSCGSPPSVA
NAVATGEAHTYESEVKLRCLEGYTMDTDTDTFTCQKDGRWFPERISCSPKKCPLPENITHILVHGDDF
SVNRQVSVSCAEGYTFEGVNISVCQLDGTWEPPFSDESCSPVSCGKPESPEHGFVVGSKYTFESTIIY
QCEPGYELEGNRERVCQENRQWSGGVAICKETRCETPLEFLNGKADIENRTTGPNVVYSCNRGYSLEG
PSEAHCTENGTWSHPVPLCKPNPCPVPFVIPENALLSEKEFYVDQNVSIKCREGFLLQGHGIITCNPD
ETWTQTSAKCEKISCGPPAHVENAIARGVHYQYGDMITYSCYSGYMLEGFLRSVCLENGTWTSPPICR
AVCRFPCQNGGICQRPNACSCPEGWMGRLCEEPICILPCLNGGRCVAPYQCDCPPGWTGSRCHTAVCQ
SPCLNGGKCVRPNRCHCLSSWTGHNCSRKRRTGF

| | |
|---|---|
| SEQ ID NO: 205 | 11158 bp |

| | | |
|---|---|---|
| NOV8o, SNP13382480 of | ORF Start: ATG at 77 | ORF Stop: TAA at 10787 |

| | | |
|---|---|---|
| CG50646-04, DNA Sequence | SNP Pos: 10749 | SNP Change: C to T |

GAATTGGTCTAGGGTCTCCCCCATTGGAATATCCATCAGTGATGAGAAATACAACGTTTGTTGAGTTT
TCTCTAGCATGAGAAGAATTTGCGCGGCTTGCTGGGGTCTGGCGCTCGTTTCGGGCTGGGCGACCTTT
CAGCAGATGTCCCCGTCGCGCAATTTCAGCTTCCGCCTCTTCCCCGAGACCGCGCCCGGGGCCCCCGG
GAGTATCCCCGCGCCGCCCGCTCCTGGCGACGAAGCGGCGGGGAGCAGAGTGGAGCGGCTGGGCCAGG
CGTTCCGCGTGCGGCTGCTGCGGGAGCTCAGCGAGCGCCTGGAGCTTGTCTTCCTGGTGGATGATTCG
TCCAGCGTGGGCGAAGTCAACTTCCGCAGCGAGCTCATGTTCGTCCGCAAGCTGCTGTCCGACTTCCC
CGTGGTGCCCACGGCCACGCGCGTGGCCATCGTGACCTTCTCGTCCAAGAACTACGTGGTGCCGCGCG
TCGATTACATCTCCACCCGCCGCGCGCGCCAGCACAAGTGCGCGCTGCTCCTCCAAGAGATCCCTGCC
ATCTCCTACCGAGGTGGCGGCACCTACACCAAGGGCGCCTTCCAGCAAGCCGCGCAAATTCTTCTTCA
TGCTAGAGAAAACTCAACAAAAGTTGTATTTCTCATCACTGATGGATATTCCAATGGGGGAGACCCTA
GACCAATTGCAGCGTCACTGCGAGATTCAGGAGTGGAGATCTTCACTTTTGGCATATGGCAAGAGAAC
ATTCGAGAGCTGAATGACATGGCTTCCACCCCAAAGGAGGAGCACTGTTACCTGCTACACAGTTTTGA
AGAATTTGAGGCTTTAGTCGCCCTCTGTCATATGTTATTTGTAGATCTACCTTCTGGGAGTTTTATTC
AAGATGATATGGTCCACTGCTCATATCTTTGTGATGAAGGCAAGGACTGCTGTGACCGAATGGGAAGC
TGCAAATGTGGGAAACACACAGGCCATTTTGAGTGCATCTGTGAAAAGGGGTATAACGGGAAAGGTCT
GCAGTATGACTGCACAGTTTGCCCATCGGGGACATACAAACCTGAAGGCTCACCAGGAGGAATCAGCA
GTTGCATTCCATGTCCTGATGAAAATCACACCTCTCCACCTGGAAGCACATCCCCTGAAGACTGTGTC
TGCAGAGAGGGATACAGGGCATCTGGCCAGACCTGTGAAGTTGTCCACTGCCCTGCCCTGAAGCCTCC
CGAAAATGGTTACTTTATCCAAAACACTTGCAACAACCACTTCAATGCAGCCTGTGGGGTCCGATGTC
ACCCTGGATTTGATCTTGTGGGAAGCAGCATCATCTTATGTCTACCCAATGGTTTGTGGTCCGGTTCA
GAGAGCTACTGCAGAGTAAGAACATGTCCTCATCTCCGCCAGCCGAAACATGGCCACATCAGCTGTTC
TACAAGGGAAATGTTATATAAGACAACATGTTTGGTTGCCTGTGATGAAGGGTACAGGCTAGAAGGCA
GTGATAAGCTTACTTGTCAAGGAAACAGCCAGTGGGATGGGCCAGAACCCCGGTGTGTGGAGCGCCAC
TGTTCCACCTTTCAGATGCCCAAAGATGTCATCATATCCCCCCACAACTGTGGCAAGCAGCCAGCCAA
ATTTGGGACGATCTGCTATGTAAGTTGCCGCCAAGGGTTCATTTTATCTGGAGTCAAAGAAATGCTGA
GATGTACCACTTCTGGAAAATGGAATGTCGGAGTTCAGGCAGCTGTGTGTAAAGACGTGGAGGCTCCT
CAAATCAACTGTCCTAAGGACATAGAGGCTAAGACTCTGGAACAGCAAGATTCTGCCAATGTTACCTG
GCAGATTCCAACAGCTAAAGACAACTCTGGTGAAAAGGTGTCAGTCCACGTTCATCCAGCTTTCACCC
CACCTTACCTTTTCCCAATTGGAGATGTTGCTATCGTATACACGGCAACTGACCTATCCGGCAACCAG
GCCAGCTGCATTTTCCATATCAAGGTTATTGATGCAGAACCACCTGTCATAGACTGGTGCAGATCTCC
ACCTCCCGTCCAGGTCTCGGAGAAGGTACATGCCGCAAGCTGGGATGAGCCTCAGTTCTCAGACAACT
CAGGTGCTGAATTGGTCATTACCAGAAGTCATACACAAGGAGACCTTTTCCCTCAAGGGGAGACTATA
GTACAGTATACAGCCACTGACCCCTCAGGCAATAACAGGACATGTGATATCCATATTGTCATAAAAGG
TTCTCCCTGTGAAATTCCATTCACACCTGTAAATGGGGATTTTATATGCACTCCAGATAATACTGGAG
TCAACTGTACATTAACTTGCTTGGAGGGCTATGATTTCACAGAAGGGTCTACTGACAAGTATTATTGT
GCTTATGAAGATGGCGTCTGGAAACCAACATATACCACTGAATGGCCAGACTGTGCCAGTAAGCGTTT
TGCAAACCACGGGTTCAAGTCCTTTGAGATGTTCTACAAAGCAGCTCGTTGTGATGACACAGATCTGA
TGAAGAAGTTTTCTGAAGCATTTGAGACGACCCTGGGAAAAATGGTCCCATCATTTTGTAGTGATGCA
GAGGACATTGACTGCAGACTGGAGGAGAACCTGACCAAAAAATATTGCCTAGAATATAATTATGACTA
TGAAAATGGCTTTGCAATTGGTCCAGGTCGCTGGGGTGCAGCTAATAGGCTGGATTACTCTTACGATG
ACTTCCTGGACACTGTGCAAGAAACAGCCACAAGCATCGGCAATGCCAAGTCCTCACGGATTAAAAGA
AGTGCCCCATTATCTGACTATAAAATTAAGTTAATTTTTAACATCACAGCTAGTGTGCCATTACCCGA
TGAAAGAAATGATACCCTTGAATGGGAAAATCAGCAACGACTCCTTCAGACATTGGAAACTATCACAA
ATAAACTGAAAAGGACTCTCAACAAAGACCCCATGTATTCCTTTCAGCTTGCATCAGAAATACTTATA
GCCGACAGCAATTCATTAGAAACAAAAAAGGCTTCCCCCTTCTGCAGACCAGGCTCAGTGCTGAGAGG
GCGTATGTGTGTCAATTGCCCTTTGGGAACCTATTATAATCTGGAACATTTCACCTGTGAAAGCTGCC
GGATCGGATCCTATCAAGATGAAGAAGGGCAACTTGAGTGCAAGCTTTGCCCCTCTGGGATGTACACG
GAATATATCCATTCAAGAAACATCTCTGATTGTAAAGCTCAGTGTAAACAAGGCACCTACTCATACAG
TGGACTTGAGACTTGTGAATCGTGTCCACTGGGCACTTATCAGCCAAAATTTGGTTCCCGGAGCTGCC
TCTCGTGTCCAGAAAACACCTCAACTGTGAAAAGAGGAGCCGTGAACATTTCTGCATGTGGAGTTCCT
TGTCCAGAAGGAAAATTCTCGCGTTCTGGGTTAATGCCCTGTCACCCATGTCCTCGTGACTATTACCA
ACCTAATGCAGGGAAGGCCTTCTGCCTGGCCTGTCCCTTTTATGGAACTACCCCATTCGCTGGTTCCA
GATCCATCACAGAATGTTCAAGTTTTAGTTCAACTTTCTCAGCGGCAGAGGAAAGTGTGGTGCCCCCT
GCCTCTCTTGGACATATTAAAAAGAGGCATGAAATCAGCAGTCAGGCAAGTCATGAATGCTTCTTTAA
CCCTTGCCACAATAGTGGAACCTGCCAGCAACTTGGGCGTGGTTATGTTTGTCTCTGTCCACTTGGAT
ATACAGGTTTAAGTGTGAAAACAGACATCGATGAGTGCAGCCCACTGCCTTGCCTCAACAATGGAGTT
TGTAAAGACCTAGTTGGGGAATTCATTTGTGAGTGCCCATCAGGTTACACAGGTAAGCACTGTGAATT
GAACATCAATGAATGTCAGTCTAATCCATGTAGAAATCAGGCCACCTGTGTGGATGAATTAAATTCAT
ACAGTTGTAAATGTCAGCCAGGATTTTCAGGCAAAAGGTGTGAAACAGGTATGTATCAACTCAGTGTT
ATTAATAACCTTAATAATGCAGTCTGTGAAGACCAGGTTGGGGGATTCTTGTGCAAATGCCCACCTGG
ATTTTTGGGTACCCGATGTGGAAAGAACGTCGATGAGTGTCTCAGTCAGCCATGCAAAAATGGAGCTA
CCTGTAAAGACGGTGCCAATAGCTTCAGGTGCCTGTGTGCAGCTGGCTTCACAGGATCACACTGTGAA
TTGAACATCAATGAATGTCAGTCTAATCCATGTAGAAATCAGGCCACCTGTGTGGATGAATTAAATTC
ATACAGTTGTAAATGTCAGCCAGGATTTTCAGGCAAAAGGTGTGAAACAGAACAGTCTACAGGCTTTA
ACCTGGATTTTGAAGTTTCTGGCATCTATGGATATGTCATGCTAGATGGCATGCTCCCATCTCTCCAT
GCTCTAACCTGTACCTTCTGGATGAAATCCTCTGACGACATGAACTATGGAACACCAATCTCCTATGC
AGTTGATAACGGCAGCGACAATACCTTGCTCCTGACTGATTATAACGGGTGGGTTCTTTATGTGAATG
GCAGGGAAAAGATAACAAACTGTCCCTCGGTGAATGATGGCAGATGGCATCATATTGCAATCACTTCG
ACAAGTACTGGTGGAGCCTGGAGGGTCTATATAAATGGGGAATTATCTGACGGTGGTACTGGCCTCTC
CATTGGCAAAGCCATACCTGGTGGCGGTGCATTAGTTCTTGGGCAAGAGCAAGACAAAAAAGGAGAGG
GGTTCAACCCGGCTGAGTCTTTTGTGGGCTCCATAAGCCAGCTCAACCTCTGGGACTATGTCCTGTCT
CCACAGCAGGTGAAGTCACTGGCTACCTCCTGCCCAGAGGAACTCAGTAAAGGAAACGTGTTAGCATG
GCCTGATTTCTTGTCAGGAATTGTGGGGAAAGTGAAGATCGATTCTAAGAGCATATTTTGTTCTGATT
GCCCACGCTTGGGAGGGTCAGTGCCTCATCTGAGAACTGCATCTGAAGATTTAAAACCAGGTTCCAAA
GTCAATCTGTTCTGTGAACCAGGCTTCCAGCTGGTCGGGAACCCTGTGCAGTACTGTCTGAATCAAGG
ACAGTGGACACAACCACTCCCCCACTGTGAACGCATTCGCTGTGGGGTGCCACCTCCTTTGGAGAATG
GCTTCCATTCAGCCGATGACTTCTATGCTGGCAGCACAGTAACCTACCAGTGCAACAATGGCTACTAT
CTATTGGGTGACTCAAGGATGTTCTGTACAGATAATGGGAGCTGGGACGGCGTTTCACCATCCTGCTT
AGATGTCGATGAGTGTGCAGTTGGATCAGATTGTAGTGAGCATGCTTCTTGCCTGAACGTAGATGGAT
CCTACATATGTTCATGTGTCCCACCGTACACAGGAGATGGGAAAAACTGTGCAGAACCTATAAAATGT
AAGGCTCCAGGAAATCCGGAAAATGGCCACTCCTCAGGTGAGATTTATACAGTAGGTGCCGAAGTCAC
ATTTTCGTGTCAGGAAGGATACCAGTTGATGGGAGTAACCAAAATCACATGTTTGGAGTCTGGAGAAT
GGAATCATCTAATACCATATTGTAAAGCTGTTTCATGTGGTAAACCGGCTATTCCAGAAAATGGTTGC
ATTGAGGAGTTAGCATTTACTTTTGGCAGCAAAGTGACATATAGGTGTAATAAAGGATATACTCTGGC
CGGTGATAAAGAATCATCCTGTCTTGCTAACAGTTCTTGGAGTCATTCCCCTCCTGTGTGTGAACCAG
TGAAGTGTTCTAGTCCGGAAAATATAAATAATGGAAAATATATTTTGAGTGGGCTTACCTACCTTTCT
ACTGCATCATATTCATGCGATACAGGATACAGCTTACAGGGCCCTTCCATTATTGAATGCACGGCTTC
TGGCATCTGGGACAGAGCGCCACCTGCCTGTCACCTCGTCTTCTGTGGAGAACCACCTGCCATCAAAG
ATGCTGTCATTACGGGGAATAACTTCACTTTCAGGAACACCGTCACTTACACTTGCAAAGAAGGCTAT
ACTCTTGCTGGTCTTGACACCATTGAATGCCTGGCCGACGGCAAGTGGAGTAGAAGTGACCAGCAGTG
CCTGGCTGTCTCCTGTGATGAGCCACCCATTGTGGACCACGCCTCTCCAGAGACTGCCCATCGGCTCT
TTGGAGACATTGCATTCTACTACTGCTCTGATGGTTACAGCCTAGCAGACAATTCCCAGCTTCTCTGC
AATGCCCAGGGCAAGTGGGTACCCCCAGAAGGTCAAGACATGCCCCGTTGTATAGCTCATTTCTGTGA
AAAACCTCCATCGGTTTCCTATAGCATCTTGGAATCTGTGAGCAAAGCAAAATTTGCAGCTGGCTCAG
TTGTGAGCTTTAAATGCATGGAAGGCTTTGTACTGAACACCTCAGCAAAGATTGAATGTATGAGAGGT
GGGCAGTGGAACCCTTCCCCCATGTCCATCCAGTGCATCCCTGTGCGGTGTGGAGAGCCACCAAGCAT
CATGAATGGCTATGCAAGTGGATCAAACTACAGTTTTGGAGCCATGGTGGCTTACAGCTGCAACAAGG
GGTTCTACATCAAAGGGGAAAAGAAGAGCACCTGCGAAGCCACAGGGCAGTGGAGTAGTCCTATACCG
ACGTGCCACCCGGTATCTTGTGGTGAACCACCTAAGGTTGAGAATGGCTTTCTGGAGCATACAACTGG
CAGGATCTTTGAGAGTGAAGTGAGGTATCAGTGTAACCCGGGCTATAAGTCAGTCGGAAGTCCTGTAT
TTGTCTGCCAAGCCAATCGCCACTGGCACAGTGAATCCCCTCTGATGTGTGTTCCTCTCGACTGTGGA
AAACCTCCCCCGATCCAGAATGGCTTCATGAAAGGAGAAAACTTTGAAGTAGGGTCCAAGGTTCAGTT
TTTCTGTAATGAGGGTTATGAGCTTGTTGGTGACAGTTCTTGGACATGTCAGAAATCTGGCAAATGGA
ATAAGAAGTCAAATCCAAAGTGCATGCCTGCCAAGTGCCCAGAGCCGCCCCTCTTGGAAAACCAGCTA
GTATTAAAGGAGTTGACCACCGAGGTAGGAGTTGTGACATTTTCCTGTAAAGAAGGGCATGTCCTGCA
AGGCCCCTCTGTCCTGAAATGCTTGCCATCCCAGCAATGGAATGACTCTTTCCCTGTTTGTAAGATTG
TTCTTTGTACCCCACCTCCCCTAATTTCCTTTGGTGTCCCCATTCCTTCTTCTGCTCTTCATTTTGGA
AGTACTGTCAAGTATTCTTGTGTAGGTGGGTTTTTCCTAAGAGGAAATTCTACCACCCTCTGCCAACC
TGATGGCACCTGGAGCTCTCCACTGCCAGAATGTGTTCCAGTAGAATGTCCCCAACCTGAGGAAATCC
CCAATGGAATCATTGATGTGCAAGGCCTTGCCTATCTCAGCACAGCTCTCTATACCTGCAAGCCAGGC
TTTGAATTGGTGGGAAATACTACCACCCTTTGTGGAGAAAATGGTCACTGGCTTGGAGGAAAACCAAC
ATGTAAAGCCATTGAGTGCCTGAAACCCAAGGAGATTTTGAATGGCAAATTCTCTTACACGGACCTAC
ACTATGGACAGACCGTTACCTACTCTTGCAACCGAGGCTTTCGGCTCGAAGGTCCCAGTGCCTTGACC
TGTTTAGAGACAGGTGATTGGGATGTAGATGCCCCATCTTGCAATGCCATCCACTGTGATTCCCCACA
ACCCATTGAAAATGGTTTTGTAGAAGGTGCAGATTACAGCTATGGTGCCATAATCATCTACAGTTGCT
TCCCTGGGTTTCAGGTGGCTGGTCATGCCATGCAGACCTGTGAAGAGTCAGGATGGTCAAGTTCCATC
CCAACATGTATGCCAATAGACTGTGGCCTCCCTCCTCATATAGATTTTGGAGACTGTACTAAACTCAA
AGATGACCAGGGATATTTTGAGCAAGAAGACGACATGATGGAAGTTCCATATGTGACTCCTCACCCTC
CTTATCATTTGGGAGCAGTGGCTAAAACCTGGGAAAATACAAAGGAGTCTCCTGCTACACATTCATCA
AACTTTCTGTATGGTACCATGGTTTCATACACCTGTAATCCAGGATATGAACTTCTGGGGAACCCTGT
GCTGATCTGCCAGGAAGATGGAACTTGGAATGGCAGTGCACCATCCTGCATTTCAATTGAATGTGACT
TGCCTACTGCTCCTGAAAATGGCTTTTTGCGTTTTACAGAGACTAGCATGGGAAGTGCTGTGCAGTAT
AGCTGTAAACCTGGACACATTCTAGCAGGCTCTGACTTAAGGCTTTGTCTAGAGAATAGAAAGTGGAG
TGGTGCCTCCCCACGCTGTGAAGCCATTTCATGCAAAAAGCCAAATCCAGTCATGAATGGATCCATCA
AAGGAAGCAACTACACATACCTGAGCACGTTGTACTATGAGTGTGACCCCGGATATGTGCTGAATGGC
ACTGAGAGGAGAACATGCCAGGATGACAAAAACTGGGATGAGGATGAGCCCATTTGCATTCCTGTGGA
CTGCAGTTCACCCCCAGTCTCAGCCAATGGCCAGGTGAGAGGAGACGAGTACACATTCCAAAAAGAGA
TTGAATACACTTGCAATGAAGGGTTCTTGCTTGAGGGAGCCAGGAGTCGGGTTTGTCTTGCCAATGGA
AGTTGGAGTGGAGCCACTCCCGACTGTGTGCCTGTCAGATGTGCCACCCCGCCACAACTGGCCAATGG
GGTGACGGAAGGCCTGGACTATGGCTTCATGAAGGAAGTAACATTCCACTGTCACGAGGGCTACATCT
TGCACGGTGCTCCAAAACTCACCTGTCAGTCAGATGGCAACTGGGATGCAGAGATTCCTCTCTGTAAA
CCAGTCAACTGTGGACCTCCTGAAGATCTTGCCCATGGTTTCCCTAATGGTTTTTCCTTTATTCATGG
GGGCCATATACAGTATCAGTGCTTTCCTGGTTATAAGCTCCATGGAAATTCATCAAGAAGGTGCCTCT
CCAATGGCTCCTGGAGTGGCAGCTCACCTTCCTGCCTGCCTTGCAGATGTTCCACACCAGTAATTGAA
TATGGAACTGTCAATGGGACAGATTTTGACTGTGGAAAGGCAGCCCGGATTCAGTGCTTCAAAGGCTT
CAAGCTCCTAGGACTTTCTGAAATCACCTGTGAAGCCGATGGCCAGTGGAGCTCTGGGTTCCCCCACT
GTGAACACACTTCTTGTGGTTCTCTTCCAATGATACCAAATGCGTTCATCAGTGAGACCAGCTCTTGG
AAGGAAAATGTGATAACTTACAGCTGCAGGTCTGGATATGTCATACAAGGCAGTTCAGATCTGATTTG
TACAGAGAAAGGGGTATGGAGCCAGCCTTATCCAGTCTGTGAGCCCTTGTCCTGTGGGTCCCCACCGT
CTGTCGCCAATGCAGTGGCAACTGGAGAGGCACACACCTATGAAAGTGAAGTGAAACTCAGATGTCTG
GAAGGTTATACGATGGATACAGATACAGATACATTCACCTGTCAGAAAGATGGTCGCTGGTTCCCTGA
ACGATTTCAGTGTGAATAGGCAAGTTTCTGTGTCATGTGCAGAAGGGTATACCTTTGAGGGAGTTAAC
ATATCAGTATGTCAGCTTGATGGAACCTGGGAGCCACCATTCTCCGATGAATCTTGCAGTCCAGTTTC
TTGTGGGAAACCTGAAAGTCCAGAACATGGATTTGTGGTTGGCAGTAAATACACCTTTGAAAGCACAA
TTATTTATCAGTGTGAGCCTGGCTATGAACTAGAGGGGAACAGGGAACGTGTCTGCCAGGAGAACAGA
CAGTGGAGTGGAGGGGTGGCAATATGCAAAGAGACCAGGTGTGAAACTCCACTTGAATTTCTCAATGG
GAAAGCTGACATTGAAAACAGGACGACTGGACCCAACGTGGTATATTCCTGCAACAGAGGCTACAGTC
TTGAAGGGCCATCTGAGGCACACTGCACAGALAATGGAACCTGGAGCCACCCAGTCCCTCTCTGCAAA
CCAAATCCATGCCCTGTTCCTTTTGTGATTCCCGAGAATGCTCTGCTGTCTGAAAAGGAGTTTTATGT
TGATCAGAATGTGTCCATCAAATGTAGGGAAGGTTTTCTGCTGCAGGGCCACGGCATCATTACCTGCA
ACCCCGACGAGACGTGGACACAGACAAGCGCCAAATGTGAAAAAATCTCATGTGGTCCACCAGCTCAC
GTAGAAAATGCAATTGCTCGAGGCGTACATTATCAATATGGAGACATGATCACCTACTCATGTTACAG
TGGATACATGTTGGAGGGTTTCCTGAGGAGTGTTTGTTTAGAAAATGGAACATGGACATCACCTCCTA
TTTGCAGAGCTGTCTGTCGATTTCCATGTCAGAATGGGGGCATCTGCCAACGCCCAAATGCTTGTTCC
TGTCCAGAGGGCTGGATGGGGCGCCTCTGTGAAGAACCAATCTGCATTCTTCCCTGTCTGAACGGAGG
TCGCTGTGTGGCCCCTTACCAGTGTGACTGCCCGCCTGGCTGGACGGGGTCTCGCTGTCATACAGCTG
TTTGCCAGTCTCCCTGCTTAAATGGTGGAAAATGTGTAAGACCAAACCGATGTCACTGTCTTTCTTCT
TGGATGGGACATAACTGTTCCAGGAAAAGGAGGACTGGGTTTTAACCACTGCACGACCATCTGGCTCT
CCCAAAAGCAGGATCATCTCTCCTCGGTAGTGCCTGGGCATCCTGGAACTTATGCAAAGAAAGTCCAA
CATGGTGCTGGGTCTTGTTTAGTAAACTTGTTACTTGGGGTTACTTTTTTTATTTTGTGATATATTTT
GTTATTCCTTGTGACATACTTTCTTACATGTTTCCATTTTTAAATATGCCTGTATTTTCTATATAAAA
ATTATATTAAATAGATGCTGCTCTACCCTCACALAATGTACATATTCTGCTGTCTATTGGGAAAGTTC
CTGGTACACATTTTTATTCAGTTACTTAAAATGATTTTTCCATTAAAGTATATTTTGCTACTAAATAA
AAAAAA

| | | |
|---|---|---|
| NOV8o, SNP13382480 of | SEQ ID NO: 206 | MW at 390042.6kD |

| | | | |
|---|---|---|---|
| CG50646-04, Protein Sequence | SNP Pos: 3558 | 3570 aa | SNP Change: Thr to Met |

MRRICAACWGLALVSGWATFQQMSPSRNFSFRLFPETAPGAPGSIPAPPAPGDEAAGSRVERLGQAFR
VRLLRELSERLELVFLVDDSSSVGEVNFRSELMFVRKLLSDFPVVPTATRVAIVTFSSKNYVVPRVDY
ISTRRARQHKCALLLQEIPAlSYRGGGTYTKGAFQQAAQILLHARENSTKVVFLITDGYSNGGDPRPI
AASLRDSGVEIFTFGIWQGNIRELNDMASTPKEEHCYLLHSFEEFEALVALCHMLFVDLPSGSFIQDD
MVHCSYLCDEGKDCCDRMGSCKCGKHTGHFECICEKGYNGKGLQYDCTVCPSGTYKPEGSPGGISSCI
PCPDENHTSPPGSTSPEDCVCREGYFASGQTCEVVHCPALKPPENGYFIQNTCNNHFNAACGVRCHPG
FDLVGSSIILCLPNGLWSGSESYCRVRTCPHLRQPKHGHISCSTREMLYKTTCLVACDEGYPLEGSDK
LTCQGNSQWDGPEPRCVERHCSTFQMPKDVIISPHNCGKQPAKFGTICYVSCRQGFILSGVKEMLRCT
TSGWNNVGVQAAVCKDVEAPQINCPKDIEAKTLEQQDSANVTWQIPTAKDNSGEKVSVHVHPAFTPPY
LFPIGDVAIVYTATDLSGNQASCIFHIKVIDAEPPVIDWCRSPPPVQVSEKVHAASWDEPQFSDNSGA
ELVITRSHTQGDLFPQGETIVQYTATDPSGNNRTCDIHIVIKGSPCEIPFTPVNGDFICTPDNTGVNC
TLTCLEGYDFTEGSTDKYYCAYEDGVWKPTYTTEWPDCASKRFANHGFKSFEMFYKAARCDDTDLMKK
FSEAFETTLGKMVPSFCSDAEDIDCRLEENLTKKYCLEYNYDYENGFAIGPGGWGAANRLDYSFDDFL
DTVQETATSIGNAKSSRIKRSAPLSDYKIKLIFNITASVPLPDERNDTLEWENQQRLLQTLETITNKL
KRTLNKDPMYSFQLASEILIADSNSLETKKASPFCRPGSVLRGRMCVNCPLGTYYNLEHFTCESCRIG
SYQDEEGQLECKLCPSGMYTEYIHSRNISDCKAQCKQGTYSYSGLETCESCPLGTYQPKFGSRSCLSC
PENTSTVKRGAVNISACGVPCPEGKFSRSGLMPCHPCPRDYYQPNAGKAFCLACPFYGTTPFAGSRSI
TECSSFSSTFSAAEESVVPPASLGHIKKRHEISSQASHECFFNPCHNSGTCQQLGRGYVCLCPLGYTG
LKCETDIDECSPLPCLNNGVCKDLVGEFICECPSGYTGKHCELNINECQSNPCRNQATCVDELNSYSC
KCQPGFSGKRCETGMYQLSVINNLNNAVCEDQVGGFLCKCPPGFLGTRCGKNVDECLSQPCKNGATCK
DGANSFRCLCAAGFTGSHCELNINECQSNPCRNQATCVDELNSYSCKCQPGFSGKRCETEQSTGFNLD
FEVSGIYGYVMLDGMLPSLHAITCTFWMKSSDDMNYGTPISYAVDNGSDNTLLLTDYNGWVLYVNGRE
KITNCPSVNDGRWHHIAITWTSTGGAWRVYINGELSDGGTGLSIGKAIPGGGALVLGQEQDKKGEGFN
PAESFVGSISQLNLWDYVLSPQQVKSLATSCPEELSKGNVLAWPDFLSGIVGKVKIDSKSIFCSDCPR
LGGSVPHLRTASEDLKPGSKVNLFCEPGFQLVGNPVQYCLNQGQWTQPLPHCERIRCGVPPPLENGFH
SADDFYAGSTVTYQCNNGYYLLGDSRMFCTDNGSWNGVSPSCLDVDECAVGSDCSEHASCLNVDGSYI
CSCVPPYTGDGKNCAEPIKCKAPGNPENGHSSGEIYTVGAEVTFSCQEGYQLMGVTKITCLESGEWNH
LIPYCKAVSCGKPAIPENGCIEELAFTFGSKVTYRCNKGYTLAGDKESSCLANSSWSHSPPVCEPVKC
SSPENINNGKYILSGLTYLSTASYSCDTGYSLQGPSIIECTASGIWDRAPPACHLVFCGEPPAIKDAV
ITGNNFTFRNTVTYTCKEGYTLAGLDTIECLADGKWSRSDQQCLAVSCDEPPIVDHASPETAHRLFGD
IAFYYCSDGYSLADNSQLLCNAQGKWVPPEGQDMPRCIAHFCEKPPSVSYSILESVSKAKFAAGSVVS
FKCMEGFVLNTSAKIECMRGGQWNPSPMSIQCIPVRCGEPPSIMNGYASGSNYSFGAMVAYSCNKGFY
IKGEKKSTCEATGQWSSPIPTCHPVSCGEPPKVENGFLEHTTGRIFESEVRYQCNPGYKSVGSPVFVC
QANRHWHSESPLMCVPLDCGKPPPIQNGFMKGENFEVGSKVQFFCNEGYELVGDSSWTCQKSGKWNKK
SNPKCMPAKCPEPPLLENQLVLKELTTEVGVVTFSCKEGHVLQGPSVLKCLPSQQWNDSFPVCKIVLC
TPPPLISFGVPIPSSALHFGSTVKYSCVGGFFLRGNSTTLCQPDGTWSSPLPECVPVECPQPEEIPNG
IIDVQGLAYLSTALYTCKPGFELVGNTTTLCGENGHWLGGKPTCKAIECLKPKEILNGKFSYTDLHYG
QTVTYSCNRGFRLEGPSALTCLETGDWDVDAPSCNAIHCDSPQPIENGFVEGADYSYGAIIIYSCFPG
FQVAGHAMQTCEESGWSSSIPTCMPIDCGLPPHIDFGDCTKLKDDQGYFEQEDDMMEVPYVTPHPPYH
LGAVAKTWENTKESPATHSSNFLYGTMVSYTCNPGYELLGNPVLICQEDGTWNGSAPSCISIECDLPT
APENGFLRFTETSMGSAVQYSCKPGHILAGSDLRLCLENRKWSGASPRCEAISCKKPNPVMNGSIKGS
NYTYLSTLYYECDPGYVLNGTERRTCQDDKNWDEDEPICIPVDCSSPPVSANGQVRGDEYTFQKEIEY
TCNEGFLLEGARSRVCLANGSWSGATPDCVPVRCATPPQLANGVTEGLDYGFMKEVTFHCHEGYILHG
APKLTCQSDGNWDAEIPLCKPVNCGPPEDLAHGFPNGFSFIHGGHIQYQCFPGYKLHGNSSRRCLSNG
SWSGSSPSCLPCRCSTPVIEYGTVNGTDFDCGKAARIQCFKGFKLLGLSEITCEADGQWSSGFPHCEH
TSCGSLPMIPNAFISETSSWKENVITYSCRSGYVIQGSSDLICTEKGVWSQPYPVCEPLSCGSPPSVA
NAVATGEAHTYESEVKLRCLEGYTMDTDTDTFTCQKDGRWFPERISCSPKKCPLPENITHILVHGDDF
SVNRQVSVSCAEGYTFEGVNISVCQLDGTWEPPFSDESCSPVSCGKPESPEHGFVVGSKYTFESTIIY
QCEPGYELEGNRERVCQENRQWSGGVAICKETRCETPLEFLNGKADIENRTTGPNVVYSCNRGYSLEG
PSEAHCTENGTWSHPVPLCKPNPCPVPFVIPENALLSEKEFYVDQNVSIKCREGFLLQGHGIITCNPD
ETWTQTSAKCEKISCGPPAHVENAIARGVHYQYGDMITYSCYSGYMLEGFLRSVCLENGTWTSPPICR
AVCRFPCQNGGICQRPNACSCPEGWMGRLCEEPICILPCLNGGRCVAPYQCDCPPGWTGSRCHTAVCQ
SPCLNGGKCVRPNRCHCLSSWMGHNCSRKRRTGF

A ClustalW comparison of the above protein sequences yields the following sequence alignment shown in Table 8B.

TABLE 8B
Comparison of the NOV8 protein sequences.

NOV8a MRRICAACWGLALVSGWATFQQMSPSRNFSFRLFPETAPGAPGSIPAPPAPGDEAAGSRV
NOV8b --------------RPWATFQQMSPSRNFSFRLFPETAPGAPGSIPAPPAPGDEAAGSRV
NOV8c --------------RPWATFQQMSPSRNFSFRLFPETAPGAPGSIPAPPAPGDEAAGSRV
NOV8d --------------RPWATFQQMSPSRNFSFRLFPETAPGAPGSIPAPPAPGDEAAGSRV
NOV8e --------------RPWATFQQMSPSRNFSFRLFPETAPGAPGSIPAPPAPGDEAAGSRV
NOV8f ------------------------------------------------------------
NOV8g ------------------------------------------------------------
NOV8h --------------MAGAPPPASLPPCSLISDCCASNQRDSVGVGPSEPGVGYSLVVRRF
NOV8i MRRICAACWGLALVSGWATFQQMSPSRNFSFRLFPETAPGAPGSIPAPPAPGDEAAGSRV
NOV8a ERLGQAFR--VRLLRELSERLELVFLVDDSSS----------------------------
NOV8b ERLGQAFRRRVRLLRELSERLELVFLVDDSSS----------------------------
NOV8c ERLGQAFRRRVRLLRELSERLELVFLVDDSSS----------------------------
NOV8d ERLGQAFRRRVRLLRELSERLELVFLVDDSSS----------------------------
NOV8e ERLGQAFRRRVRLLRELSERLELVFLVDDSSS----------------------------
NOV8f ------------------------------------------------------------
NOV8g ------------------------------------------------------------
NOV8h LSRSEKRNIRVGVTRFSSYTLAGLDTIECLADGKWSRSDQQCLAVSCDEPPIVDHASPET
NOV8i ERLGQAFR--VRLLRELSERLELVFLVDDSSS----------------------------
NOV8a ----------------------------VGEVNFRSELMFVRKLLSDFPVVPTATRVAIV
NOV8b ----------------------------VGEVNFRSELMFVRKLLSDFPVVPTATRVAIV
NOV8c ----------------------------VGEVNFRSELMFVRKLLSDFPVVPTATRVAIV
NOV8d ----------------------------VGEVNFRSELMFVRKLLSDFPVVPTATRVAIV
NOV8e ----------------------------VGEVNFRSELMFVRKLLSDFPVVPTATRVAIV
NOV8f ------------------------------------------------------------
NOV8g ------------------------------------------------------------
NOV8h AHRLFGDIAFYYCSDGYSLADNSQLLCNAQGKWVPPEGQDMPRCIAHFCEKPPSVSYSIL
NOV8i ----------------------------VGEVNFRSELMFVRKLLSDFPVVPTATRVAIV
NOV8a TFSSKN------------------------------YVVPRVDYISTRRARQHKCALLLQ
NOV8b TFSSKN------------------------------YVVPRVDYISTPRARQHKCALLLQ
NOV8c TFSSKN------------------------------YVVPRVDYISTRPARQHKCALLLQ
NOV8d TFSSKN------------------------------YVVPRVDYISTRRARQHKCALLLQ
NOV8e TFSSKN------------------------------YVVPRADYISTRRARQHKCALLLQ
NOV8f ------------------------------------------MSIQCIPVRCGEPPSINN
NOV8g ------------------------------------------------------------
NOV8h ------------------------------------------------------------
NOV8i TFSSKN------------------------------YVVPRVDYISTRRARQHKCALLLQ
NOV8a EIPAISYRGGGTYTKGAFQQAAQILLHARENSTKVVFLITDGYSNGGDPRPIAASLRDSG
NOV8b EIPAISYRGGGTYTKGAFQQAAQILLHARENSTKVVFLITDGYSNGGDPRPIAASLRDSG
NOV8c EIPAISYRGGGTYTKGAFQQAAQILLHARENSTKVVFLITDGYSNGGDPRPIAASLRDSG
NOV8d EIPAISYRGGGTYTKGAFQQAAQILLHARENSTKVVFLITDGYSNGGDPRPIAASLRDSG
NOV8e EIPAISYRGGGTYTKGAFQQAAQILLHARENSTKVVFLITDGYSNGGDPRPIAASLRDSG
NOV8f GYASGSNYSFGAMVAYSCNKGFYIKGEKYSTCEATGQWSSPIPTCHPVSCGEPPKVENGF
NOV8g --------------------------------------------MLSSVIQIALTCFLPV
Nov8h GYASGSNYSFGAMVAYSCNKGFYIKGEKYSTCEATGQWSSPIPTCHPVSCGEPPKVENGF
NOV8i EIPAISYRGGGTYTKGAFQQAAQILLHARENSTKVVFLITDGYSNGGDPRPIAASLRDSG
NOV8a VEIFTFGIWQGNIRELNDMASTPKEEHCYLLHSFEEFEALVALCHMLFVDLPSGSFIQDD
NOV8b VEIFTFGIWQGNIRELNDMASTPKEEHCYLLHSFEEFEALAR--RALHEDLPSGSFIQDD
NOV8c VEIFTFGIWQGNIRELNDMASTPKEEHCYLLHSFEEFEALAR--RALHEDLPSGSFIQDD
NOV8d VEIFTFGIWQGNIRELNDMASTPKEEHCYLLHSFEEFEALAR--RALHEDLPSGSFIQDD
NOV8e VEIFTFGIWQGNIRELNDMASTPKEEHCYLLHSFEEFEALAR--RALHEDLPSGSFIQDD
NOV8f LEHTTGRIFESEVRYQCNPGYKSVGSPVFVCQANRHWHSESP-LMCVPLDCGKPPPIQNG
NOV8g AQHTTGRIFESEVRYQCNPGYKSVGSPVFVCQANRHWHSESP-LMCVPLDCGKPPPIQNG
NOV8h LEHTTGRIFESEVRYQCNPGYKSVGSPVFVCQANRHWHSESP-LMCVPLDCGKPPPIQNG
N0V8i VEIFTFGIWQGNIRELNDMASTPKEEHCYLLHSFEEFEALAR--RALHEDLPSGSFIQDD
NOV8a MVHCSYLCDEGKDCCDRMGSCKCGKHTGHFECICEKGYNGKG----LQYDCTVCPSGTYK
NOV8b MVHCSYLCDEGKDCCDRMGSCKCGTHTGHFECICEKGYNGKG----LQYECTACPSGTYK
NOV8c MVHCSYLCDEGKDCCDRMGSCKCGTHTDRFECICEKGYYGKG----LQYECTACPSGTYK
NOV8d MVHCSYLCDEGKDCCDRMGSCKCGTHTGHFECICEKGYYGKG----LQYECTACPSGTYK
NOV8e MVHCSYLCDEGKDCCDRNGSCKCGTHTGHFECICEKGYYGKG----LQYECTACPSGTYK
NOV8f FMKGENFEVGSKVQFFCNEGY-ELVGDSSWTCQKSGKWNKKSNPKCMPAKCPEPPLLENQ
NOV8g FMKGENFEVGSKVQFFCNEGY-ELVGDSSWTCQKSGKWNKKSNPKCMPAKCPEPPLLENQ
NOV8h FMKGENFEVGSKGQFFCNEGLXSFVGDSSWTCQKSGKWNKKSNPKCMPAKCPEPPLLENQ
NOV8i MVHCSYLCDEGKDCCDRMGSCKCGTHTGHFECICEKGYYGKG----LQYECTACPSGTYK
NOV8a PEGSPGGISSCIPCPDENHTSPPGSTSPEDCVCREGYRASGQTCEVVHCPALKPPENGYF
NOV8b PEASPGGISSCIPCPDENHTSPPGSTSPEDCVCREGYPASGQTCELVHCPALKPPENGYF
NOV8c PEGSPGGISSCIPCPDENHTSPPGSTSPEDCVCREGYRASGQTCELVHCPAWKPPENGYF
NOV8d PEGSPGGISSCIPCPDENHTSPPGSTSPEDCVCREGYRASGQTCELVHCPALKPPENGYF
NOV8e PEGSPGGISSCIPCPDENHTSPPGSTSPEDCVCREGYRASGQTCELVHCPALKPPENGYF
NOV8f LVLKELTTEVGVVTFSCKEGHVLQGPSVLKCLPSQQWNDSFPVCKIVLCTPPPLISFGVP
NOV8g LVLKELTTEVGVVTFSCKEGHVLQGPSVLKCLPSQQWNDSFPVCKIVLCTPPPLISFGVP
NOV8h LVLKELTTEVGVVTFSCKERHVLQGPSVLKCLPSQQWNDSFPVCKIVLCTPPPLISFGVP
NOV8i PEASPGGISSCIPCPDENHTSPPGSTSPEDCVCREGYRASGQTCELVHCPALKPPENGYF
NOV8a IQNTCNNHFNAACGVRCHPGFDLVGSSIILCLPNGLWSGSESYCRVRTCPHLRQPKHGHI
NOVBb IQNTCNNHFNAACGVRCHPGFDLVGSSIILCLPNGLWSGSESYCRVRTCPHLRQPKHGHI
NOV8c IQNTCNNHFNAACGVRCHPGFDLVGSSIILCLPNGLWSGSESYCRVRTCPHLRQPKHGHI
NOV8d IQNTCNNHFNAACGVRCHPGFDLVGSSIILCLPNGLWSGSESYCRVRTCPHLRQPKHGHI
NOV8e IQNTCNMHFNAACGVRCHPGFDLVGSSIILCLPNGLWSGSESYCRVRTCPHLRQPKHGHI
NOV8f IP-SSALHFGSTVKYSCVGGFFLRGNSTTLCQPDGTWSSPLPECVPVECPQPEEIPNGII
NOV8g IP-SSALHFGSTVKYSCVGGFFLRGNSTTLCQPDGTWSSPLPECVPVECPQPEETPNGII
NOV8h IP-SSALHFGSTVKYSCVGGFFLRGNSTTLCQPDGTWSSPLPECVPVECPQPEEIPNGII
NOV8i IQNTCNNEFNAACGVRCHPGFDLVGSSIILCLPNGLWSGSESYCRVRTCPHLRQPKHGHI
NOV8a SCSTREMLYKTTCLVACDEGYRLEGSDKLTCQGNSQWDGPEPRCVERHCSTFQMPKDVII
NOV8b SCSTREMLYKTTCLVACDEGYRLEGSDKLTCQGNSQWDGPEPRCVERHCSTFQMPKDVII
NOV8c SCSTREMLYKTTCLVACDEGYRLEGSDKLTCQGNSQWDGPEPRCVERHCSTFQMPKDIII
NOV8d SCSTREMLYKTTCLVACDEGYRLEGSDKLTCQGNSQWDGPEPRCVERHCSTFQMPKDVII
NOV8e SCSTREMLYKTTCLVACDEGYRLEGSDKLTCQGNSQWDGPEPRCVERHCSTFQMPKDIII
NOV8f DVQG--LAYLSTALYTCKPGFELVGNTTTLCGENGHWLGGKP---T--CKAIECLKPKEI
NOV8g DVQG--LAYLSTALYTCKPGFELVGNTTTLCGENGHWLGGKP---T--CKAIECLKPKEI
NOV8h DVQG--LAYLSTALYTCKPGFELVGNTTTLCGENGHWLGGKP---T--CKAIECLKPKEI
NOV8i SCSTREMLYKTTCLVACDEGYRLEGSDKLTCQGNSQWDGPEPRCVERHCSTFQMPKDVII
NOV8a SPHNCGKQPAKFGTICYVSCRQGFILSGVKEMLRCTTSGKWNVGVQAAVCKDVEAPQINC
NOV8b SPHNCGKQPAKFGTICYVSCRQGFILSGVKEMLRCTTSGKWNVGVQAAVCKDVEAPQINC
NOV8c SPHNCGKQPAKFGTICYVSCRQGFILSGVKEMLRCTTSGKWNVGVQAAVCKDVEAPQINC
NOV8d SPHNCGKQPAKFGTICYVSCRQGFILSGVKEMLRCTTSGKWNVGVQAAVCKDVEAPQINC
NOV8e SPHNCGKQPAKFGTICYVSCRQGFILSGVKEMLRCTTSGKWNVGVQAAVCKDVEAPQINC
NOV8f LNGKFSYTDLHYGQTVTYSCNRGFRLEGS-QCLDLFRDR---------------------
NOV8g LNGKFSYTDLHYGQTVTYSCNRGFRLEGS-QCLDLFRDR---------------------
NOV8h LNGKFSYTDLHYGQTVTYSCNRGFRLEGP-SALTCLETGDWDVDAPSCNAIHCDSPQPIE
NOV8i SPHNCGKQPAKFGTICYVSCRQGFILSGVKEMLRCTTSGKWNVGVQAAVCKDVEAPQINC
NOV8a PKDIEAKTLEQ-------------------------------------------------
NOV8b PKDIEAKTLEQ-------------------------------------------------
NOV8c PKDIEAKTLEQ-------------------------------------------------
NOV8d PKDIEAKTLEQ-------------------------------------------------
NOV8e PKDIEAKTLEQ-------------------------------------------------
NOV8f ------------------------------------------------------------
NOV8g ------------------------------------------------------------
NOV8h NGFVEGADYSYGAIIIYSCFPGFQVAGHAMQTCEESGWSSSIPTCMPIDCGLPPHIDFGD
NOV8i PKDIEAKTLEQ-------------------------------------------------
NOV8a ------------------------------------------------------------
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h CTKLKDDQGYFEQEDDMMEVPYVTPHPPYHLGAVAKTWENTKESPATHSSNFLYGTMVSY
NOV8i ------------------------------------------------------------
NOV8a --------------------------QDSANVTWQIPTAKDN-----SGEKVSVHVHPAF
NOV8b --------------------------QDSANVTWQIPTAKDN-----SGEKVSVHVHPAF
NOV8c --------------------------QDSANVTWQIPTAKDN-----SGEKVSVHVHPAF
NOV8d --------------------------QDSANVTWQIPTAKDN-----SGEKVSVHVHPAF
NOV8e --------------------------QDSANVTWQIPTAKDN-----SGEKVSVHVHPAF
NOV8f ------------------------------------------------------------
NOV8g ------------------------------------------------------------
NOV8h TCNPGYELLGNPVLICQEDGTWNGSAPSCISIECDLPTAPENGFLRFTETSMGSAVQYSC
NOV8i --------------------------QDSANVTWQIPTAKDN-----SGEKVSVHVHPAF
NOV8a TPPYLFPIGDVAIVYTATDLSGN-------------------------------------
NOV8b TPPYLFPIGDVAIVYTATDLSGN-------------------------------------
NOV8c TPPYLFPIGDVAIVYTATDLSGN-------------------------------------
NOV8d TPPYLFPIGDVAIVYTATDLSGN-------------------------------------
NOV8e TPPYLFPIGDVAIVYTATDLSGN-------------------------------------
NOV8f ------------------------------------------------------------
NOV8g ------------------------------------------------------------
NOV8h KPGHILAGSDLRLCLENRXWSGASPRCEAISCKKPNPVMNGSIKGSNYTYLSTLYYECDP
NOV8i TPPYLFPIGDVAIVYTATDLSGN-------------------------------------
NOV8a --------QASCIFHIKVIDAEPPVIDWCRSPPPVQVSEKVHAASWD-------------
NOV8b --------QASCIFHIKVIDAEPPVIDWCRSPPPVQVSEKVHAASWD-------------
NOV8c --------QASCIFHIKVIDAEPPVIDWCRSPPPVQVSEKVHAASWD-------------
NOV8d --------QASCIFHIKVIDAEPPVIDWCRSPPPVQVSEKVHAASWD-------------
NOV8e --------QASCIFHIKVIDAEPPVIDWCRSPPPVQVSEKVHAASWD-------------
NOV8f ------------------------------------------------------------
NOV8g ------------------------------------------------------------
NOv8h GYVLNGTERRTCQDDKNWDEDEPICIPVDCSSPPVSANGQVRGDEYTFQKEIEYTCNEGF
NOV8i --------QASCIFHIKVIDAEPPVIDWCRSPPPVQVSEKVHAASWD-------------
NOV8a ------------------------------------------------------------
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h LLEGARSRVCLANGSWSGATPDCVPVRCATPPQLANGVTEGLDYGFMKEVTFHCHEGYIL
NOV8i ------------------------------------------------------------
NOV8a ------------------------------------------------------------
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h HGAPKLTCQSDGNWDAEIPLCKPVNCGPPEDLAHGFPNGFSFIHGGHIQYQCFPGYKLHG
NOV8i ------------------------------------------------------------
NOV8a ------------------------------------------------------------
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h NSSRRCLSNGSWSGSSPSCLPCRCSTPVIEYGTVNGTDFDCGKAARIQCFKGFKLLGLSE
NOV8i ------------------------------------------------------------
NOV8a ------------------------------------------------------------
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ITCEADGQWSSGFPHCEHTSCGSLPMIPNAFISETSSWKENVITYSCRSGYVIQGSSDLI
NOV8i ------------------------------------------------------------
NOV8a ------------------------------------------------------------
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h CTEKGVWSQPYPVCEPLSCGSPPSVANAVATGEAHTYESEVKLRCLEGYTMDTDTDTFTC
NOV8i ------------------------------------------------------------
NOV8a ------------------------------------------------------------
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h QKDGRWFPERISCSPKKCPLPENITHILVHGDDFSVNRQVSVSCAEGYTFEGVNISVCQL
NOV8i ------------------------------------------------------------
NOV8a ----EPQFSDNSGAELVITRSHTQGDLFPQGE---------------TIVQYTATDPSGN
NOV8b ----EPQFSDNSGAELVITRSHTQGDLFPQGE---------------TIVQYTATDPSGN
NOV8c ----EPQFSDNSGAELVITRSHTQGDLFPQGE---------------TIVQYTATDPSGN
NOV8d ----EPQFSDNSGAELVITRSHTQGDLFPQGE---------------TIVQYTATDPSGN
NOV8e ----EPQFSDNSGAELVITRSHTQGELFPQGE---------------TIVQYTATDPSGN
NOV8f ------------------------------------------------------------
NOV8g ------------------------------------------------------------
NOV8h DGTWEPPFSDESCSPVSCGKPESPEHGFVVGSKYTFESTIIYQCEPGYELENLAVNPSGP
NOV8i ----EPQFSDNSGAELVITRSHTQGDLFPQGE---------------TIVQYTATDPSGN
NOV8a ---------------------------------------NRTCDIHIVIKGSPCEIPFTP
NOV8b ---------------------------------------NRTCDIHIVILE---------
NOV8c ---------------------------------------NRTCDIHIVILE---------
NOV8d ---------------------------------------NRTCDIHIVILE---------
NOV8e ---------------------------------------NRTCDIHIVILE---------
NOV8f ------------------------------------------------------------
NOV8g ------------------------------------------------------------
NOV8h GLFLVDRTLSCRSELARGPIQTLFAWVSAAEGAEQRTLVNRKCCCLIIPLEVLSQRNTRP
NOV8i ---------------------------------------NRICDIHIVMKGSPCEIPFTP
NOV8a VNGDFICTPDNTGVNCTLTCLEGYDFTEGSTDKYYCAYEDGVWKPTYTTEWPDCASKRFA
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h CEVSVRPYWGGNRERVCQENRQWSGGVAICKETRCETPLEFLNGKADIENRTTGPNVVYS
NOV8i VNGDFICTPDNTGVNCTLTCLEGYDFTEGSTDKYYCAYEDGVWKPTYTTEWPDCASKRFA
NOV8a NHGFKSFEMFYKAARCDDTDLMKKFSEAFETTLGKMVPSFCSDAEDIDCRLEENLTKKYC
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h CNRGYSLEGPSEAHCTENGTWSHPVPLCKPNPCPVPFVIPENALLSEKEFYVDQNVSIKC
NOV8i NHGFKSFEMFYKAARCDDSDLMKKFSEAFETTLGKMVPSFCSDAEDIDCRLEENLTKKYC
NOV8a LEYNYDYENGFAIGPGGWGAANRLDYSYDDFLDTVQETATSIGNAKSSRIKRSAPLSDYK
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h REGFLLQGHGIITCNPDETWTQTSAKCERRYTQQPKSLNFQLAAYCSIRMFILRGGVQDG
NOV8i LEYNYDYENGFAIGPGGWGAANRLDYSYDDFLDTVQETATSIGNAKSSRIKRSAPLSDYK
NOV8a IKLIFNITASVPLPDERNDTLEWENQQRLLQTLETITNKLKRTLNKDPMYSFQLASEILI
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h QLETAVAGASHREEQKQKREKARWYNGPPGSHMGQAELPPPAKGGGPPCGNFSNSSQGFM
NOV8i IKLIFNITASVPLPDERNDTLEWENQQRLLQTLETITNKLKRTLNKDPMYSFQLASEILI
NOV8a ADSNSLETKKASPFCRPGSVLRGRMCVNCPLGTYYNLEHFTCESCRIGSYQDEEGQLECK
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h NRPLISLRWSPWGSMWPWSPQISRLSPSPAGSEESRQAGLVGFPTAQFTdSAKGQLERFV
NOV8i ADSNSLETKKASPFCRPGSVLRGRMCVNCPLGTYYNLEHFTCESCRIGSYQDEEGQLECK
NOV8a LCPSGMYTEYIHSRNISDCKAQCKQGTYSYSGLETCESCPLGTYQPKFGSRSCLSCPENT
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h KRVPDPMPPDWDETPPQQGSRMRPPHNRGHQTPYTRVFLLASGQCPSGTELPEERAGSHL
NOV8i LCPSGMYTEYIHSRNISDCKAQCKQGTYSYSGLETCESCPLGTVQPKFGSRSCLSCPENT
NOV8a STVKRGAVNISACGVPCPEGKFSRSGLMPCHPCPRDYYQPNAGKAFCLACPFYGTTPFAG
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h CCSAASAGKAQNWAEARIDELKEVGFRKWVIMKFAELKEHVLNQCKDAKNQDKTLQDPLT
NOV8i STVKRGAVNISACGVPCPEGKFSRSGLMPCHPCPRDYYQPNAGKAFCLACPFYGTTPFAG
NOV8a SRSITECSSFSSTFSAAEESVVPPASLGHIKKRHEISSQASHECFFNPCHNSGTCQQLGR
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h RITSLERNVNDLMELKNTTRELHNATTKQGQHSSSGNPENPSKILHEKINPKTHNPQVLQ
NOV8i SRSITECSSFSSTFSAAEESVVPPASLGHIKKRHEISSQASHECFFNPCHNSGTCQQLGR
NOV8a GYVCLCPLGYTGLKCETDIDECSPLPCLNNGVCKDLVGEFICECPSGYTGKHCELNINEC
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h EISCGPPAHVENAIARGVHYQYGDMITYSCYSGYMLEGFLRSVCLENGTWTSPPICRAVC
NOV8i GYVCLCPLGYTGLKCETDIDECSPLPCLNNGVCKDLVGEFICECPSGYTGKHCELNINEC
NOV8a QSNPCRNQATCVDELNSYSCKCQPGFSGKRCETGMYQLSVINNLNNAVCEDQVGGFLCKC
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h RFPCQNGGICQRPNACSCPEGWMGRLCEEPICILPCLNGGRCVAPYQCDCPPGWTGSRCH
Nov8i QSNPCRNQATCVDELNSYSCKCQPGFSGKRCETGMYQLSVINNLNNAVCEDQVGGFLCKC
NOV8a PPGFLGTRCGKNVDECLSQPCKNGATCKDGANSFRCLCAAGFTGSHCELNINECQSNPCR
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h TGRPLSWFVFLVAQAHETPEDIEECDLDSEVVAK--------------------------
NOV8i PPGFLGTRCGKNVDECLSQPCKNGATCKDGANSFRCLCAAGFTGSHCELNINECQSNPCR
NOV8a NQATCVDELNSYSCKCQPGFSGKRCETEQSTGFNLDFEVSGIYGYVMLDGMLPSLHALTC
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i NQATCVDELNSYSCKCQPGFSGKRCETEQSTGFNLDFEVSGIYGYVMLDGMLPSLHALTC
NOV8a TFWMKSSDDMNYGTPISYAVDNGSDNTLLLTDYNGWVLYVNGREKITNCPSVNDGRWHHI
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i TFWMKSSDDMNYGTPISYAVDNGSDNTLLLTDYNGWVLYVNGREKITNCPSVNDGRWHHI
NOV8a AITWTSTGGAWRVYINGELSDGGTGLSIGKAIPGGGALVLGQEQDKKGEGFNPAESFVGS
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i AITWTSTGGAWRVYINGELSDGGTGLSIGKAIPGGGALVLGQEQDKKGEGFNPAESFVGS
NOV8a ISQLNLWDYVLSPQQVKSLATSCPEELSKGNVLAWPDFLSGIVGKVKIDSKSIFCSDCPR
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i ISQLNLWDYVLSPQQVKSIATSCPEELSKGNVLAWPDFLSGIVGKVKIDSKSIFCSDCPR
NOV8a LGGSVPHLRTASEDLKPGSKVNLFCEPGFQLVGNPVQYCLNQGQWTQPLPHCERIRCGVP
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i LGGSVPHLRTASEDLKPGSKVNLFCEPGFQLVGNPVQYCLNQGQWTQPLPHCERIRCGVP
NOV8a PPLENGFHSADDFYAGSTVTYQCNNGYYLLGDSPMFCTDNGSWNGVSPSCLDVDECAVGS
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i PPLENGFHSADDFYAGSTVTYQCNNGYYLLGDSRMFCTDNGSWNGVSPSCLDVDECAVGS
NOV8a DCSEHASCLNVDGSYICSCVPPYTGDGKNCAEPIKCKAPGNPENGHSSGEIYTVGAEVTF
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i DCSEHASCLNVDGSYICSCVPPYTGDGKNCAEPIKCKAPGNPENGHSSGEIYTVGAEVTF
NOV8a SCQEGYQLMGVTKITCLESGEWNHLIPYCKAVSCGKPAIPENGCIEELAFTFGSKVTYRC
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i SCQEGYQLMGVTKITCLESGEWNHLIPYCKAVSCGKPATPENGCIEELAFTFGSKVTYRC
NOV8a NKGYTLAGDKESSCLANSSWSHSPPVCEPVKCSSPENINNGKYILSGLTYLSTASYSCDT
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i NKGYTLAGDKESSCLANSSWSHSPPVCEPVKCSSPENINNGKYILSGLTYLSTASYSCDT
NOV8a GYSLQGPSIIECTASGIWDRAPPACHLVFCGEPPAIKDAVITGNNFTFRNTVTYTCKEGY
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i GYSLQGPSIIECTASGIWDRAPPACHLVFCGEPPAIKDAVITGNNFTFRNTVTYTCKEGY
NOV8a TLAGLDTIECLADGKWSRSDQQCLAVSCDEPPIVDHASPETAHRLFGDIAFYYCSDGYSL
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i TLAGLDTIECLADGKWSRSDQQCLAVSCDEPPIVDHASPETAHRLFGDIAFYYCSDGYSL
NOV8a ADNSQLLCNAQGKWVPPEGQDMPRCIAHFCEKPPSVSYSILESVSKAKFAAGSVVSFKCM
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i ADNSQLLCNAQGKWVPPEGQDMPRCIAHFCEKPPSVSYSILESVSKAKFAAGSVVSFKCM
NOV8a EGFVLNTSAKIECMRGGQWNPSPMSIQCIPVRCGEPPSIMNGYASGSNYSFGAMVAYSCN
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i EGFVLNTSAKIECMRGGQWNPSPMSIQCIPVRCGEPPSIMNGYASGSNYSFGAMVAYSCN
NOV8a KGFYIKGEKKSTCEATGQWSSPIPTCHPVSCGEPPKVENGFLEHTTGRIFESEVRYQCNP
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i KGFYIKGEKKSTCEATGQWSSPIPTCHPVSCGEPPKVENGFLEHTTGRIFESEVRYQCNP
NOV8a GYKSVGSPVFVCQANRHWHSESPLMCVPLDCGKPPPIQNGFMKGENFEVGSKVQFFCNEG
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i GYKSVGSPVFVCQANRHWHSESPLMCVPLDCGKPPPIQNGFMKGENFEVGSKVQFFCNEG
NOV8a YELVGDSSWTCQKSGKWNKKSNPKCMPAKCPEPPLLENQLVLKELTTEVGVVTFSCKEGH
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i YELVGDSSWTCQKSGKWNKKSNPKCMPAKCPEPPLLENQLVLKELTTEVGVVTFSCKEGH
NOV8a VLQGPSVLKCLPSQQWNDSFPVCKIVLCTPPPLISFGVPIPSSALHFGSTVKYSCVGGFF
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i VLQGPSVLKCLPSQQWNDSFPVCKIVLCTPPPLISFGVPIPSSALHFGSTVKYSCVGGFF
NOV8a LRGNSTTLCQPDGTWSSPLPECVPVECPQPEEIPNGIIDVQGLAYLSTALYTCKPGFELV
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i LRGNSTTLCQPDGTWSSPLPECVPVECPQPEEIPNGIIDVQGLAYLSTALYTCKPGFELV
NOV8a GNTTTLCGENGHWLGGKPTCKAIECLKPKEILNGKFSYTDLHYGQTVTYSCNRGFRLEGP
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i GNTTTLCGENGHWLGGKPTCKAIECLKPKEILNGKFSYTDLHYGQTVTYSCNRGFRLEGP
NOV8a SALTCLETGDWDVDAPSCNAIHCDSPQPIENGFVEGADYSYGAIIIYSCFPGFQVAGHAM
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i SALTCLETGDWDVDAPSCNAIHCDSPQPIENGFVEGADYSYGAIIIYSCFPGFQVAGHAM
NOV8a QTCEESGWSSSIPTCMPIDCGLPPHIDFGDCTKLKDDQGYFEQEDDMMEVPYVTPHPPYH
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i QTCEESGWSSSIPTCMPIDCGLPPHIDFGDCTKLKDDQGYFEQEDDMMEVPYVTPHPPYH
NOV8a LGAVAKTWENTKESPATHSSNFLYGTMVSYTCNPGYELLGNPVLICQEDGTWNGSAPSCI
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i LGAVAKTWENTKESPATHSSNFLYGTMVSYTCNPGYELLGNPVLICQEDGTWNGSAPSCI
NOV8a SIECDLPTAPENGFLRFTETSMGSAVQYSCKPGHILAGSDLRLCLENRKWSGASPRCEAI
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i SIECDLPTAPENGFLRFTETSMGSAVQYSCKPGHILAGSDLRLCLENRKWSGASPRCEAI
NOV8a SCKKPNPVNNGSIKGSNYTYLSTLYYECDPGYVLNGTERRTCQDDKNWDEDEPICIPVDC
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i SCKKPNPVMNGSIKGSNYTYLSTLYYECDPGYVLNGTERRTCQDDKNWDEDEPICIPVDC
NOV8a SSPPVSANGQVRGDEYTFQKEIEYTCNEGFLLEGARSRVCLANGSWSGATPDCVPVRCAT
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i SSPPVSANGQVRGDEYTFQKEIEYTCNEGFLLEGARSRVCLANGSWSGATPDCVPVRCAT
NOV8a PPQLANGVTEGLDYGFMKEVTFHCHEGYILHGAPKLTCQSDGNWDAEIPLCKPVNCGPPE
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i PPQLANGVTEGLDYGFMKEVTFHCHEGYILHGAPKLTCQSDGNWDAEIPLCKPVNCGPPE
NOV8a DLAHGFPNGFSFIHGGHIQYQCFPGYKLHGNSSRRCLSNGSWSGSSPSCLPCRCSTPVIE
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i DLAHGFPNGFSFIHGGHIQYQCFPGYKLHGNSSRRCLSNGSWSGSSPSCLPCRCSTPVIE
NOV8a YGTVNGTDFDCGKAARIQCFKGFKLLGLSEITCEADGQWSSGFPHCEHTSCGSLPMIPNA
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i YGTVNGTDFDCGKAARIQCFKGFKLLGLSEITCEADGQWSSGFPHCEHTSCGSLPMIPNA
NOV8a FISETSSWKENVITYSCRSGYVIQGSSDLICTEKGVWSQPYPVCEPLSCGSPPSVANAVA
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i FISETSSWKENVITYSCRSGYVIQGSSDLICTEKGVWSQPYPVCEPLSCGSPPSVANAVA
NOV8a TGEAHTYESEVKLRCLEGYTMDTDTDTFTCQKDGRWFPERISCSPKKCPLPENITHILVH
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i TGEAHTYESEVKLRCLEGYTMDTDTDTFTCQKDGRWFPERISCSPKKCPLPENITHILVH
NOV8a GDDFSVNRQVSVSCAEGYTFEGVNISVCQLDGTWEPPFSDESCSPVSCGKPESPEHGFVV
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i GDDFSVNRQVSVSCAEGYTFEGVNISVCQLDGTWEPPFSDESCSPVSCGKPESPEHGFVV
NOV8a GSKYTFESTIIYQCEPGYELEGNRERVCQENRQWSGGVAICKETRCETPLEFLNGKADIE
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i GSKYTFESTIIYQCEPGYELEGNRERVCQENRQWSGGVAICKETRCETPLEFLNGKADIE
NOV8a NRTTGPNVVYSCNRGYSLEGPSEAHCTENGTWSHPVPLCKPNPCPVPFVIPENALLSEKE
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i NRTTGPNVVYSCNRGYSLEGPSEAHCTENGTWSHPVPLCKPNPCPVPFVIPENALLSEKE
NOV8a FYVDQNVSIKCREGFLLQGHGIITCNPDETWTQTSAKCEKISCGPPAHVENATARGVHYQ
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i FYVDQNVSIKCREGFLLQGHGIITCNPDETWTQTSAKCEKISCGPPAHVENAIARGVHYQ
NOV8a YGDMITYSCYSGYMLEGFLRSVCLENGTWTSPPICRAVCRFPCQNGGICQRPNACSCPEG
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i YGDMITYSCYSGYMLEGFLRSVCLENGTWTSPPICRAVCRFPCQNGGICQRPNACSCPEG
NOV8a WMGRLCEEPICILPCLNGGRCVAPYQCDCPPGWTGSRCHTAVCQSPCLNGGKCVRPNRCH
NOV8b ------------------------------------------------------------
NOV8c ------------------------------------------------------------
NOV8d ------------------------------------------------------------
NOV8e ------------------------------------------------------------
NOV8f ------------------------------------------------------------
NOV8g ------------------------------------------------------------
NOV8h ------------------------------------------------------------
NOV8i WMGRLCEEPICILPCLNGGRCVAPYQCDCPPGWTGSRCHTAVCQSPCLNGGKCVRPNRCH
NOV8a CLSSWTGHNCSRKRRTGF
NOV8b ------------------
NOV8c ------------------
NOV8d ------------------
NOV8e ------------------
NOV8f ------------------
NOV8g ------------------
NOV8h ------------------
NOV8i CLSSWTGHNCSRKRRTGF
NOV8a (SEQ ID NO: 178)
NOV8b (SEQ ID NO: 180)
NOV8c (SEQ ID NO: 182)
NOV8d (SEQ ID NO: 184)
NOV8e (SEQ ID NO: 186)
NOV8f (SEQ ID NO: 188)
NOV8g (SEQ ID NO: 190)
NOV8h (SEQ ID NO: 192)
NOV8i (SEQ ID NO: 194)

Further analysis of the NOV8a protein yielded the following properties shown in Table 8C.

TABLE 8C
Protein Sequence Properties NOV8a

| | |
|---|---|
| SignalP analysis: | Cleavage site between residues 17 and 18 |
| PSORT II analysis: | |

| PSG: | a new signal peptide prediction method | |
|---|---|---|
| | N-region: | length 3; pos. chg 2; neg. chg 0 |
| | H-region: | length 23; peak value 8.53 |
| | PSG score: | 4.12 |

| | |
|---|---|
| GvH: | von Heijne's method for signal seq recognition |
| | GvH score (threshold: −2.1): −2.66 |
| | possible cleavage site: between 16 and 17 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 2 | |
| | Number of TMS(s) for threshold 0.5: 0 | |
| | PERIPHERAL | Likelihood = 0.58 (at 253) |

| | ALOM score: −0.16 (number of TMSs: 0) | | | |
|---|---|---|---|---|
| MTOP: | Prediction of membrane topology (Hartmann et al.) | | | |
| | Center position for calculation: 6 | | | |
| | Charge difference: −2.0 C(1.0)-N(3.0) | | | |
| | N >= C: N-terminal side will be inside | | | |
| MITDISC: | discrimination of mitochondrial targeting seq | | | |
| | R content: | 4 | Hyd Moment(75): | 11.27 |
| | Hyd Moment(95): | 11.90 | G content: | 2 |
| | D/E content: | 1 | S/T content: | 5 |

| | |
|---|---|
| | Score: 0.60 |
| Gavel: | prediction of cleavage sites for mitochondrial preseq |
| | R-10 motif at 37 SRN FS |

NUCDISC: discrimination of nuclear localization signals

| | |
|---|---|
| | pat4: KKRH (3) at 1183 |
| | pat4: RKRR (5) at 3564 |
| | pat7: none |
| | bipartite: none |
| | content of basic residues: 8.0% |
| | NLS Score: 0.03 |
| KDEL: | ER retention motif in the C-terminus: none |

ER Membrane Retention Signals:

XXRR-like motif in the N-terminus: RRIC

none
SKL: peroxisomal targeting signal in the C-terminus: none
PTS2: 2nd peroxisomal targeting signal: found
RLGGSVPHL at 1632
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 76.7 |
| COIL: | Lupas's algorithm to detect coiled-coil regions |
| | total: 0 residues |

Final Results (k = 9/23):

```
65.2%: nuclear
21.7%: mitochondrial
  8.7%: cytoplasmic
  4.3%: vacuolar
```

>> prediction for CG50646-04 is nuc (k = 23)

A search of the NOV8a protein against the Geneseq database, a proprietary database that contains sequences published in patents and patent publication, yielded several homologous proteins shown in Table 8D.

TABLE 8D
Geneseq Results for NOV8a

| | | NOV8a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| ABJ10589 | Human novel protein NOV2a SEQ ID | 1 . . . 3570 | 3570/3570 (100%) | 0.0 |
| | NO: 6 - Homo sapiens, 3570 aa. | 1 . . . 3570 | 3570/3570 (100%) | |
| | [WO200259315-A2, 01-AUG-2002] | | | |
| ABJ10590 | Human novel protein NOV2b SEQ ID | 1 . . . 3570 | 3551/3570 (99%) | 0.0 |
| | NO: 8 - Homo sapiens, 3568 aa. | 1 . . . 3568 | 3555/3570 (99%) | |
| | [WO200259315-A2, 01-AUG-2002] | | | |
| AAE20146 | Human C3b/C4b complement | 3 . . . 3570 | 3502/3570 (98%) | 0.0 |
| | receptor-like protein - Homo sapiens, | 4 . . . 3571 | 3519/3570 (98%) | |
| | 3571 aa. [WO200210388-A2, 07- | | | |
| | FEB-2002] | | | |
| AAE20147 | Mouse C3b/C4b complement | 3 . . . 3569 | 2886/3570 (80%) | 0.0 |
| | receptor-like protein - Mus musculus, | 31 . . . 3593 | 3174/3570 (88%) | |
| | 3594 aa. [WO200210388-A2, 07- | | | |
| | FEB-2002] | | | |
| AAM93954 | Human polypeptide, SEQ ID NO: | 2074 . . . 3570 | 1485/1497 (99%) | 0.0 |
| | 4153 - Homo sapiens, 1497 aa. | 1 . . . 1497 | 1487/1497 (99%) | |
| | [EP1130094-A2, 05-SEP-2001] | | | |

In a BLAST search of public sequence databases, the NOV8a protein was found to have homology to the proteins shown in the BLASTP data in Table 8E.

TABLE 8E
Public BLASTP Results for NOV8a

| | | NOV8a | | | |
|---|---|---|---|---|---|
| Protein | | Residues/ | Identities/ | | |
| Accession | | Match | Similarities for the | | Expect |
| Number | Protein/Organism/Length | Residues | Matched Portion | | Value |
| Q9ES77 | Polydom protein precursor - Mus | 3 . . . 3569 | 2894/3570 | (81%) | 0.0 |
| | musculus (Mouse), 3567 aa. | 4 . . . 3566 | 3180/3570 | (89%) | |
| Q8NBT9 | Hypothetical protein FLJ90754 - | 2074 . . . 3570 | 1485/1497 | (99%) | 0.0 |
| | Homo sapiens (Human), 1497 aa. | 1 . . . 1487 | 1487/1497 | (99%) | |
| Q96JU7 | Hypothetical protein FLJ14964 - | 231 . . . 1546 | 1255/1316 | (95%) | 0.0 |
| | Homo sapiens (Human), 1316 aa. | 1 . . . 1314 | 1267/1316 | (95%) | |
| Q8C720 | Polydomain protein - Mus | 3 . . . 845 | 694/846 | (82%) | 0.0 |
| | musculus (Mouse), 848 aa | 4 . . . 846 | 748/846 | (88%) | |
| | (fragment). | | | | |
| Q922H0 | Polydomain protein - Mus | 2902 . . . 3569 | 534/668 | (79%) | 0.0 |
| | musculus (Mouse), 669 aa | 2 . . . 668 | 594/668 | (87%) | |
| | (fragment). | | | | |

PFam analysis predicts that the NOV8a protein contains the domains shown in the Table 8F.

TABLE 8F
Domain Analysis of NOV8a

| | | Identities/ | | |
|---|---|---|---|---|
| | | Similarities | | |
| Pfam Domain | NOV8a Match Region | for the Matched Region | | Expect Value |
| vwa | 80 . . . 256 | 57/200 | (28%) | 5.1e−37 |
| | | 128/200 | (64%) | |
| Metallothio_PEC | 269 . . . 324 | 22/80 | (28%) | 0.15 |
| | | 27/80 | (34%) | |
| sushi | 377 . . . 432 | 18/64 | (28%) | 1e−06 |
| | | 39/64 | (61%) | |
| sushi | 437 . . . 492 | 21/64 | (33%) | 4.6e−14 |
| | | 48/64 | (75%) | |
| sushi | 497 . . . 558 | 12/68 | (18%) | 0.17 |
| | | 39/68 | (57%) | |
| HYR | 560 . . . 641 | 29/86 | (34%) | 6.1e−16 |
| | | 57/86 | (66%) | |
| HYR | 642 . . . 721 | 27/86 | (31%) | 3.4e−16 |
| | | 54/86 | (63%) | |
| TNFR_c6 | 1017 . . . 1055 | 14/48 | (29%) | 0.74 |
| | | 34/48 | (71%) | |
| EGF | 1196 . . . 1227 | 18/47 | (38%) | 3.9e−07 |
| | | 24/47 | (51%) | |
| EGF | 1234 . . . 1265 | 18/47 | (38%) | 1.2e−10 |
| | | 27/47 | (57%) | |
| EGF | 1272 . . . 1303 | 17/47 | (36%) | 6.9e−10 |
| | | 28/47 | (60%) | |
| EGF | 1348 . . . 1379 | 13/47 | (28%) | 1.3e−08 |
| | | 26/47 | (55%) | |
| EGF | 1386 . . . 1417 | 17/47 | (36%) | 6.9e−10 |
| | | 28/47 | (60%) | |
| pentaxin | 1430 . . . 1622 | 57/209 | (27%) | 5e−14 |
| | | 99/209 | (47%) | |
| sushi | 1630 . . . 1684 | 18/64 | (28%) | 2.3e−10 |
| | | 42/64 | (66%) | |
| sushi | 1689 . . . 1742 | 25/63 | (40%) | 3.3e−17 |
| | | 43/63 | (68%) | |
| EGF | 1748 . . . 1782 | 14/47 | (30%) | 1.8e−05 |
| | | 26/47 | (55%) | |
| sushi | 1788 . . . 1841 | 24/63 | (38%) | 2e−15 |
| | | 43/63 | (68%) | |
| sushi | 1846 . . . 1899 | 20/63 | (32%) | 4.2e−14 |
| | | 45/63 | (71%) | |
| sushi | 1904 . . . 1957 | 22/63 | (35%) | 8.5e−14 |
| | | 42/63 | (67%) | |
| sushi | 1962 . . . 2015 | 22/63 | (35%) | 1.9e−13 |
| | | 39/63 | (62%) | |
| sushi | 2020 . . . 2077 | 19/64 | (30%) | 5.4e−07 |
| | | 42/64 | (66%) | |
| sushi | 2082 . . . 2140 | 17/65 | (26%) | 2.3e−08 |
| | | 39/65 | (60%) | |
| sushi | 2145 . . . 2198 | 24/63 | (38%) | 3.8e−18 |
| | | 46/63 | (73%) | |
| sushi | 2203 . . . 2258 | 19/64 | (30%) | 3.3e−10 |
| | | 45/64 | (70%) | |
| sushi | 2263 . . . 2317 | 26/63 | (41%) | 9.4e−17 |
| | | 46/63 | (73%) | |
| sushi | 2322 . . . 2375 | 21/63 | (33%) | 3.2e−08 |
| | | 41/63 | (65%) | |
| sushi | 2380 . . . 2434 | 24/63 | (38%) | 1.9e−13 |
| | | 43/63 | (68%) | |
| sushi | 2439 . . . 2492 | 21/63 | (33%) | 3.8e−11 |
| | | 42/63 | (67%) | |
| sushi | 2497 . . . 2550 | 26/63 | (41%) | 3.4e−15 |
| | | 43/63 | (68%) | |
| sushi | 2555 . . . 2607 | 22/63 | (35%) | 1.1e−12 |
| | | 38/63 | (60%) | |
| sushi | 2659 . . . 2711 | 21/65 | (32%) | 2.9e−10 |
| | | 43/65 | (66%) | |
| sushi | 2716 . . . 2769 | 19/63 | (30%) | 9.6e−10 |
| | | 38/63 | (60%) | |
| sushi | 2774 . . . 2827 | 20/63 | (32%) | 5.2e−14 |
| | | 45/63 | (71%) | |
| sushi | 2832 . . . 2885 | 22/63 | (35%) | 1.1e−14 |
| | | 42/63 | (67%) | |
| sushi | 2890 . . . 2943 | 21/63 | (33%) | 7.6e−12 |
| | | 35/63 | (56%) | |
| sushi | 2948 . . . 3001 | 19/63 | (30%) | 7.6e−13 |
| | | 40/63 | (63%) | |
| sushi | 3006 . . . 3058 | 21/62 | (34%) | 3.8e−08 |
| | | 37/62 | (60%) | |
| sushi | 3063 . . . 3116 | 22/63 | (35%) | 5.7e−11 |
| | | 44/63 | (70%) | |
| sushi | 3121 . . . 3175 | 17/64 | (27%) | 1.1e−10 |
| | | 40/64 | (62%) | |
| sushi | 3180 . . . 3235 | 16/62 | (26%) | 1.2e−05 |
| | | 39/62 | (63%) | |
| sushi | 3240 . . . 3293 | 19/63 | (30%) | 1.2e−12 |
| | | 44/63 | (70%) | |
| sushi | 3298 . . . 3351 | 23/63 | (37%) | 1.6e−10 |
| | | 41/63 | (65%) | |
| sushi | 3356 . . . 3410 | 18/64 | (28%) | 3.6e−09 |
| | | 41/64 | (64%) | |
| sushi | 3415 . . . 3467 | 22/63 | (35%) | 1.5e−12 |
| | | 42/63 | (67%) | |
| EGF | 3467 . . . 3498 | 13/47 | (28%) | 0.0029 |
| | | 22/47 | (47%) | |
| EGF | 3503 . . . 3530 | 18/47 | (38%) | 2e−05 |
| | | 24/47 | (51%) | |
| EGF | 3535 . . . 3562 | 13/47 | (28%) | 0.0034 |
| | | 22/47 | (47%) | |

### Example 9

The NOV9 clone was analyzed, and the nucleotide and encoded polypeptide sequences are shown in Table 9A.

TABLE 9A
NOV9 Sequence Analysis

| | | |
|---|---|---|
| NOV9a, CG50736-09 | SEQ ID NO: 207 | 3260 bp |
| DNA Sequence | ORF Start: ATG at 171 | ORF Stop: TGA at 2862 |

GGCACGAGCAGGAGCTTCCCAAGAACCCGAAAACTTCCCAGTATTTCTTCCAGTTGCAGGAGCATTTC
GTGAAAGATCTGGTCGGCCCAGGCCCCTTCACTGTTTTTGCACCTTTATCTGCAGCCTTTGATGAGGA
AGCTCGGGTTAAAGACTGGGACAAATACGGTTTAATGCCCCAGGTTCTTCGGTACCATGTGGTCGCCT
GCCACCAGCTGCTTCTGGAAAACCTGAAATTGATCTCAAATGCTACTTCCCTCCAAGGAGAGCCAATA
GTCATCTCCGTCTCTCAGAGCACGGTGTATATAAATAATAAGGCTAAGATCATATCCAGTGATATCAT
CAGTACTAATGGGATTGTTCATATCATAGACAAATTGCTATCTCCCAAAAATTTGCTTATCACTCCCA
AAGACAACTCTGGAAGAATTCTGCAAAATCTTACGACTTTGGCAACAAACAATGGCTACATCAAATTT
AGCAACTTAATACAGGACTCAGGTTTGCTGAGTGTCATCACCGATCCCATCCACACCCCAGTCACTCT
CTTCTGGCCCACCGACCAAGCCCTCCATGCCCTACCTGCTGAACAACAGGACTTCCTGTTCAACCAAG
ACAACAAGGACAAGCTGAAGGAGTATTTGAAGTTTCATGTGATACGAGATGCCAAGGTTTTAGCTGTG
GATCTTCCCACATCCACTGCCTGGAAGACCCTGCAAGGTTCAGAGCTGAGTGTGAAATGTGGAGCTGG
CAGGGACATCGGTGACCTCTTTCTGAATGGCCAAACCTGCAGAATTGTGCAGCGGGAGCTCTTGTTTG
ACCTGGGTGTGGCCTACGGCATTGACTGTCTGCTGATTGATCCCACCCTGGGGGGCCGCTGTGACACC
TTTACTACTTTCGATGCCTCGGGGGAGTGTGGGAGCTGTGTCAATACTCCCAGCTGCCCAAGGTGGAG
TAAACCAAAGGGTGTGAAGCAGAAGTGTCTCTACAACCTGCCCTTCAAGAGGAACCTGGAAGGCTGCC
GGGAGCGGTGCAGCCTGGTGATACAGATCCCCAGGTGCTGCAAGGGCTACTTCGGGCGAGACTGTCAG
GCCTGCCCTGGAGGACCAGATGCCCCGTGTAATAACCGGGGTGTCTGCCTTGATCAGTACTCGGCCAC
CGGAGAGTGTAAATGCAACACCGGCTTCAATGGGACGGCGTGTGAGATGTGCTGGCCGGGGAGATTCG
GGCCTGATTGTCTGCCCTGTGGCTGCTCAGACCACGGACAGTGCGATGATGGCATCACGGGCTCCGGG
CAGTGCCTCTGTGAAACGGGGTGGACAGGCCCCTCGTGTGACACTCAGGCAGTTTTGCCTGCAGTGTG
TACGCCTCCTTGTTCTGCTCATGCCACCTGTAAGGAGAACAACACGTGTGAGTGTAACCTGGATTATG
AAGGTGACGGAATCACATGCACAGTTGTGGATTTCTGCAAACAGGACAACGGGGGCTGTGCAAAGGTG
GCCAGATGCTCCCAGAAGGGCACGAAGGTCTCCTGCAGCTGCCAGAAGGGATACAAAGGGGACGGGCA
CAGCTGCACAGAGATAGACCCCTGTGCAGACGGCCTTAACGGAGGGTGTCACGAGCACGCCACCTGTA
AGATGACAGGCCCGGGCAAGCACAAGTGTGAGTGTAAAAGTCACTATGTCGGAGATGGGCTGAACTGT
GAGCCGGAGCAGCTGCCCATTGACCGCTGCTTACAGGACAATGGGCAGTGCCATGCAGACGCCAAATG
TGTCGACCTCCACTTCCAGGATACCACTGTTGGGGTGTTCCATCTACGCTCCCCACTGGGCCAGTATA
AGCTGACCTTTGACAAAGCCAGAGAGGCCTGTGCCAACGAAGCTGCGACCATGGCAACCTACAACCAG
CTCTCCTATGCCCAGAAGGCCAAGTACCACCTGTGCTCAGCAGGCTGGCTGGAGACCGGGCGGGTTGC
CTACCCCACAGCCTTCGCCTCCCAGAACTGTGGCTCTGGTGTGGTTGGGATAGTGGACTATGGACCTA
GACCCAACAAGAGTGAAATGTGGGATGTCTTCTGCTATCGGATGAAAGATGTGAACTGCACCTGCAAG
GTGGGCTATGTGGGAGATGGCTTCTCATGCAGTGGGAACCTGCTGCAGGTCCTGATGTCCTTCCCCTC
ACTCACAAACTTCCTGACGGAAGTGCTGGCCTATTCCAACAGCTCAGCTCGAGGCCGTGCATTTCTAG
AACACCTGACTGACCTGTCCATCCGCGGCACCCTCTTTGTGCCACAGAACAGTGGGCTGGGGGAGAAT
GAGACCTTGTCTGGGCGGGACATCGAGCACCACCTCGCCAATGTCAGCATGTTTTTCTACAATGACCT
TGTCAATGGCACCACCCTGCAAACGAGGGTGGGAAGCAAGCTGCTCATCACTGCCAGCCAGGACCCAC
TCCAACCGACGGAGACCAGGTTTGTTGATGGAAGAGCCATTCTGCAGTGGGACATCTTTGCCTcCAAT
GGGATCATTCATGTCATTTCCAGGCCTTTAAAAGCACCCCCTGCCCCCGTGACCTTGACCCACACTGG
CTTGGGAGCAGGGATCTTCTTTGCCATCATCCTGGTGACTGGGGCTGTTGCCTTGGCTGCTTACTCCT
ACTTTCGGATAAACCGGAGAACAATCGGCTTCCAGCATTTTGAGTCGGAAGAGGACATTAATGTTGCA
GCTCTTGGCAAGCAGCAGCCTGAGAATATCTCGAACCCCTTGTATGAGAGCACAACCTCAGCTCCCCC
AGAACCTTCCTACGACCCCTTCACGGACTCTGAAGAACGGCAGCTTGAGGGCAATGACCCCTTGAGGA
CACTGTGAGGGCCTGGACGGGAGATGCCAGCCATCACTCACTGCCACCTGGGCCATCAACTGTGAATT
CTCAGCACCAGTTGCCTTTTAGGAACGTAAAGTCCTTTAAGCACTCAGAAGCCATACCTCATCTCTCT
GGCTGATCTGGGGGTTGTTTCTGTGGGTGAGAGATGTGTTGCTGTGCCCACCCAGTACAGCTTCCTCC
TCTGACCCTTTGGCTCTTCTTCCTTTGTACTCTTCAGCTGGCACCTGCTCCATTCTGCCCTACATGAT
GGGTAACTGTGATCTTTCTTCCCTGTTAGATTGTAAGCCTCCNTCTTTGTATCCCAGCCCCTAGCCCA
GTGCCTGACACAGGAACTGTGCACAATAAGGTTTATGGACAGAAAAAAAAAAAAAAAAAAAAAA

| | | | |
|---|---|---|---|
| NOV9a, CG50738-09 | SEQ ID NO: 208 | 897 aa | MW at 97584.6kD |
| Protein Sequence | | | |

MPQVLRYHVVACHQLLLENLKLISNATSLQGEPIVISVSQSTVYINNKAKIISSDIISTNGIVHIIDK
LLSPKNLLITPKDNSGRILQNLTTLATNNGYIKFSNLIQDSGLLSVITDPIHTPVTLFWPTDQALHAL
PAEQQDFLFNQDNKDKLKEYLKFHVIRDAKVLAVDLPTSTAWKTLQGSELSVKCGAGRDIGDLFLNGQ
TCRIVQRELLFDLGVAYGIDCLLIDPTLGGRCDTFTTFDASGECGSCVNTPSCPRWSKPKGVKQKCLY
NLPFKRNLEGCRERCSLVIQIPRCCKGYFGRDCQACPGGPDAPCNNRGVCLDQYSATGECKCNTGFNG
TACEMCWPGRFGPDCLPCGCSDHGQCDDGITGSGQCLCETGWTGPSCDTQAVLPAVCTPPCSAHATCK
ENNTCECNLDYEGDGITCTVVDFCKQDNGGCAKVARCSQKGTKVSCSCQKGYKGDGHSCTEIDPCADG
LNGGCHEHATCKMTGPGKHKCECKSHYVGDGLNCEPEQLPIDRCLQDNGQCHADAKCVDLHFQDTTVG
VFHLRSPLGQYKLTFDKAREACANEAATMATYNQLSYAQKAKYHLCSAGWLETGRVAYPTAFASQNCG
SGVVGIVDYGPRPNKSEMWDVFCYRMKDVNCTCKVGYVGDGFSCSGNLLQVLMSFPSLTNFLTEVLAY
SNSSARGRAFLEHLTDLSIRGTLFVPQNSGLGENETLSGRDIEHHLANVSMFFYNDLVNGTTLQTRVG
SKLLITASQDPLQPTETRFVDGRAILQWDIFASNGIIHVISRPLKAPPAPVTLTHTGLGAGIFFAIIL
VTGAVALAAYSYFRINRRTIGFQHFESEEDINvAALGKQQPENISNPLYESTTSAPPEPSYDPFTDSE
ERQLEGNDPLRTL

| | | |
|---|---|---|
| NOV9b, 197408749 | SEQ ID NO: 209 | 1668 bp |
| DNA Sequence | ORF Start: at 1 | ORF Stop: end of sequence |

GGATCCAGAATTCTGCAAAATCTTACGACTTTGGCAACAAACAATGGCTACATCAAATTTAGCAACTT
AATACAGGACTCAGGTTTGCTGAGTGTCATCACCGATCCCATCCACACCCCAGTCACTCTCTTCTGGC
CCACCGACCAAGCCCTCCATGCCCTACCTGCTGAACAACAGGACTTCCTGTTCAACCAAGACAACAAG
GACAAGCTGAAGGAGTATTTGAAGTTTCATGTGATACGAGATGCCAAGGTTTTAGCTGTGGATCTTCC
CACATCCACTGCCTGGAAGACCCTGCAAGGTTCAGAGCTGAGTGTGAAATGTGGAGCTGGCAGGGACA
TCGGTGACCTCTTTCTGAATGGCCAAACCTGCAGAATTGTGCAGCGGGAGCTCTTGTTTGACCTGGGT
GTGGCCTACGGCATTGACTGTCTGCTGATTGATCCCACCCTGGGGGGCCGCTGTGACACCTTTACTAC
TTTCGATGCCTCGGGGGAGTGTGGGAGCTGTGTCAATACTCCCAGCTGCCCAAGGTGGAGTAAACCAA
AGGGTGTGAAGCAGAAGTGTCTCTACAACCTGCCCTTCAAGAGGAACCTGGAAGGCTGCCGGGAGCGG
TGCAGCCTGGTGATACAGATCCCCAGGTGCTGCAAGGGCTACTTCGGGCGAGACTGTCAGGCCTGCCC
TGGAGGACCAGATGCCCCGTGTAATAACCGGGGTGTCTGCCTTGATCAGTACTCGGCCACCGGAGAGT
GTAAATGCAACACCGGCTTCAATGGGACGGCATGTGAGATGTGCTGGCCGGGGAGATTTGGGCCTGAT
TGTCTGCCCTGTGGCTGCTCAGACCACGGACAGTGCGATGATGGCATCACGGGCTCCGGGCAGTGCCT
CTGTGAAACGGGGTGGACAGGCCCCTCGTGTGACACTCAGGCAGTTTTGCCTGCAGTGTGTACGCCTC
CTTGTTCTGCTCATGCCACCTGTAAGGAGAACAACACGTGTGAGTGTAACCTGGATTATGAAGGTGAC
GGAATCACATGCACAGTTGTGGATTTCTGCAAACAGGACAACGGGGGCTGTGCAAAGGTGGCCAGATG
CTCCCAGAAGGGCACGAAGGTCTCCTGCAGCTGCCAGAAGGGATACAAAGGGGACGGGCACAGCTGCA
CAGAGATAGACCCCTGTGCAGACGGCCTTAACGGAGGGTGTCACGAGCACGCCACCTGTAAGATGACA
GGCCCGGGCAAGCACAAGTGTGAGTGTAIAAGTCACTATGTCGGAGATGGGCTGAACTGTGGGCCGGA
GCAGCTGCCCATTGACCGCTGCTTACAGGACAATGGGCAGTGCCATGCAGACGCCAAATGTGTCGACC
TCCACTTCCAGGATACCACTGTTGGGGTGTTCCATCTACGCTCCCCACTGGGCCAGTATAAGCTGACC
TTTGACAAAGCCAGAGAGGCCTGTGCCAACGAAGCTGCGACCATGGCAACCTACAACCAGCTCTCCTA
TGCCCAGAAGGCCAAGTACCACCTGTGCTCAGCAGGCTGGCTGGAGACCGGGCGGGTTGCCTACCCCA
CAGCCTTCGCCTCCCAGAACTGTGGCTCTGGTGTGGTTGGGATAGTGGACTATGGACCTAGACCCAAC
AAGAGTGAAATGTGGGATGTCTTCTGCTATCGGCCG

| | | | |
|---|---|---|---|
| NOV9b, 197408749 | SEQ ID NO: 210 | 556 aa | MW at 60171.4kD |
| Protein Sequence | | | |

GSRILQNLTTLATNNGYIKFSNLIQDSGLLSVITDPIHTPVTLFWPTDQALHALPAEQQDFLFNQDNK
DKLKEYLKFHVIRDAKVLAVDLPTSTAWKTLQGSELSVKCGAGRDIGDLFLNGQTCRIVQRELLFDLG
VAYGIDCLLIDPTLGGRCDTFTTFDASGECGSCVNTPSCPRWSKPKGVKQKCLYNLPFKPNLEGCRER
CSLVIQIPRCCKGYFGRDCQACPGGPDAPCNNRGVCLDQYSATGECKCNTGFNGTACEMCWPGRFGPD
CLPCGCSDHGQCDDGITGSGQCLCETGWTGPSCDTQAVLPAVCTPPCSAHATCKENNTCECNLDYEGD
GITCTVVDFCKQDNGGCAKVARCSQKGTKVSCSCQKGYKGDGHSCTEIDPCADGLNGGCHEHATCKMT
GPGKHKCECKSHYVGDGLNCGPEQLPIDRCLQDNGQCHADAKCVDLHFQDTTVGVFHLRSPLGQYKLT
FDKAREACANEAATMATYNQLSYAQKAKYHLCSAGWLETGRVAYPTAFASQNCGSGVVGIVDYGPRPN
KSEMWDVFCYRP

| | | |
|---|---|---|
| NOV9c, CG50736-01 | SEQ ID NO: 211 | 2011 bp |
| DNA Sequence | ORF Start: ATG at 877 | ORF Stop: TGA at 1822 |

GGGTGTGATGGGCTTCTAGTTTCTCTAGCTGCATCACCCTTGAACCATCCAGAGTCCCAGTAAGCCAC
GGGCTTGAGCATGGAGGAGAATCCTCAGAGACAGAACCCCTGCCCACATGTCTGGGCCTTGCTCAAGC
CAGCAAGGGGCTGAATCCCTGTGTTTCAGGACTCAGGTTTGCTGAGTGTCATCACCGATCCCATCCAC
ACCCCAGTCACTCTCTTCTGGCCCACCGACCAAGCCCTCCATGCCCTACCTGCTGAACAACAGGACTT
CCTGTTCAACCAAGACAACAAGGACAAGCTGAAGGAGTATTTGAAGTTTCATGTGATACGAGATGCCA
AGGTTTTAGCTGTGGATCTTCCCACATCCACTGCCTGGAAGACCCTGCAAGGTTCAGAGCTGAGTGTG
AAATGTGGAGCTGGCAGGGACATCGGTGACCTCTTTCTGAATGGCCAAACCTGCAGAATTGTGCAGCG
GGAGCTCTTGTTTGACCTGGGTGTGGCCTACGGCATTGACTGTCTGCTGATTGATCCCACCCTGGGGG
GCCGCTGTGACACCTTTACTACTTTCGATGCCTCGGGGGAGTGTGGGAGCTGTGTCAATACTCCCAGC
TGCCCAAGGTGGAGTAAACCAAAGGGTGTGAAGCAGAAGTGTCTCTACAACCTGCCCTTCAAGAGGAA
CCTCGAAGGCTGCCGGGAGCGATGCAGCCTGGTGATACAGATCCCCAGGTGCTGCAAGGGCTACTTCG
GGCGAGACTGTCAGGCCTGCCCTGGAGGACCAGATGCCCCGTGTAATAACCGGGGTGTCTGCCTTGAT
CAGTACTCGGCCACCGGAGAGTGTAAATGCAACACCGGCTTCAATGGGACGGCGTGTGAGATGTGCTG
GCCGGGGAGATTTGGGCCTGATTGTCTGCCCTGTGGCTGCTCAGACCACGGACAGTGCGATGATGGCA
TCACGGGCTCCGGGCAGTGCCTCTGTGAAACGGGGTGGACAGGCCCCTCGTGTGACACTCAGGCAGTT
TTGTCTGCAGTGTGTACGCCTCCTTGTTCTGCTCATGCCACCTGTAAGGAGAACAACACGTGTGAGTG
TAACCTGGATTATGAAGGTGACGGAATCACATGCACAGTTGTGGATTTCTGCAAACAGGACAACGGGG
GCTGTGCAAAGGTGGCCAGATGCTCCCAGAAGGGCACGAAGGTCTCCTGCAGCTGCCAGAAGGGATAC
AAAGGGGACGGGCACAGCTGCACAGAGATAGACCCCTGTGCAGACGGCCTTAACGGAGGGTGTCACGA
GCACGCCACCTGTAAGATGACAGGCCCGGGCAAGCACAAGTGTGAGTGTAAAAGTCACTATGTCGGAG
ATGGGCTGAACTGTGAGCCGGAGCAGCTGCCCATTGACCGCTGCTTACAGGACAATGGGCAGTGCCAT
GCAGACGCCAAATGTGCCGACCTCCACTTCCAGGATACCACTGTTGGGGTGTTCCATCTACGCTCCCC
ACTGGGCCAGTATAAGCTGACCTTTGACAAAGCCAGAGAGGCCTGTGCCAACGAAGCTGCGACCATGG
CAACCTACAACCAGCTCTCCTATGCCCAGAAGGCCAAGTACCACCTGTGCTCAGCAGGCTGGCTGGAG
ACCGGGCGGGTTGCCTACCCCACAGCCTTCGCCTCCCAGAACTGTGGCTCTGGTGTGGTTGGGATAGT
GGACTATGGACCCAGACCCAACAAGAGTGAAATGTGGGATGTCTTCTGCTATCGGATGAAAGGAAGTG
CTGGCCTATTCCAACAGCTCAGCTCGAGGCCGTGCATTTCTAGAACACCTGACTGACCTGTCCATCCG
CGGCACCCTCTTTGTGCCACAGAACAGTGGGCTGGGGGAGAATGAGACCTTGTCTGGGCGGGACATCG
AACACCACCTCGCCAATGTCAGCATGTTTTTCTACAATGACCTTGTCAATGGCACCACCCTGCAAACG
AGGCTGGGAAGCAAGCTGCTCATCACTGCCAGCCAGGAC

| | | | |
|---|---|---|---|
| NOV9c,CG50736-01 | SEQ ID NO: 212 | 315 aa | MW at 33739.5kD |
| Protein Sequence | | | |

MCWPGRFGPDCLPCGCSDHGQCDDGITGSGQCLCETGWTGPSCDTQAVLSAVCTPPCSAHATCKENNT
CECNLDYEGDGITCTVVDFCKQDNGGCAKVARCSQKGTKVSCSCQKGYKGDGHSCTEIDPCADGLNGG
CHEHATCKMTGPGKHKCECKSHYVGDGLNCEPEQLPIDRCLQDNGQCHADAKCADLHFQDTTVGVFHL
RSPLGQYKLTFDKAREACANEAATMATYNQLSYAQKAKYHLCSAGWLETGRVAYPTAFASQNCGSGVV
GIVDYGPRPNKSEMWDVFCYRMKGSAGLFQQLSSRPCISRTPD

| | | |
|---|---|---|
| NOV9d, CG50736-02 | SEQ ID NO: 213 | 1804 bp |
| DNA Sequence | ORF Start: ATG at 877 | ORF Stop: TGA at 1609 |

GGGTGTGATGGGCTTCTAGTTTCTCTAGCTGCATCACCCTTGAACCATCCAGAGTCCCAGTAAGCCAC
GGGCTTGAGCATGGAGGAGAATCCTCAGAGACAQAACCCCTGCCCACATGTCTGGGCCTTGCTCAAGC
CAGCAAGGGGCTGAATCCCTGTGTTTCAGGACTCAGGTTTGCTGAGTGTCATCACCGATCCCATCCAC
ACCCCAGTCACTCTCTTCTGGCCCACCGACCAAGCCCTCCATGCCCTACCTGCTGAACAACAGGACTT
CCTGTTCAACCAAGACAACAAGGACAAGCTGAAGGAGTATTTGAAGTTTCATGTGATACGAGATGCCA
AGGTTTTAGCTGTGGATCTTCCCACATCCACTGCCTGGAAGACCCTGCAAGGTTCAGAGCTGAGTGTG
AAATGTGGAGCTGGCAGGGACATCGGTGACCTCTTTCTGAATGGCCAAACCTGCAGAATTGTGCAGCG
GGAGCTCTTGTTTGACCTGGGTGTGGCCTACGGCATTGACTGTCTGCTGATTGATCCCACCCTGGGGG
GCCGCTGTGACACCTTTACTACTTTCGATGCCTCGGGGGAGTGTGGGAGCTGTGTCAATACTCCCAGC
TGCCCAAGGTGGAGTAAACCAAAGGGTGTGAAGCAGAAGTGTCTCTACAACCTGCCCTTCAAGAGGAA
CCTGGAAGGCTGCCGGGAGCGATGCAGCCTGGTGATACAGATCCCCAGGTGCTGCAAGGGCTACTTCG
GGCGAGACTGTCAGGCCTGCCCTGGAGGACCAGATGCCCCGTGTAATAACCGGGGTGTCTGCCTTGAT
CAGTACTCGGCCACCGGAGAGTGTAAATGCAACACCGGCTTCAATGGGACGGCGTGTGAGATGTGCTG
GCCGGGGAGATTTGGGCCTGATTGTCTGCCCTGTGGCTGCTCAGACCACGGACAGTGCGATGATGGCA
TCACGGGCTCCGGGCAGTGCCTCTGTGAAACGGGGTGGACAGGCCCCTCGTGTGACACTCAGGCAGTT
TTGTCTGCAGTGTGTACGCCTCCTTGTTCTGCTCATGCCACCTGTAAGGAGAACAACACGTGTGAGTG
TAACCTGGATTATGAAGGTGACGGAATCACATGCACAGTTGTGGATTTCTGCAAACAGGACAACGGGG
GCTGTGCAAAGGTGGCCAGATGCTCCCAGAAGGGCACGAAGGTCTCCTGCAGCTGCCAGAAGGGATAC
AAAGGGGACGGGCACAGCTGCACAGAGATAGACCCCTGTGCAGACGGCCTTAACGGAGGGTGTCACGA
GCACGCCACCTGTAAGATGACAGGCCCGGGCAAGCACAAGTGTGAGTGTAAAAGTCACTATGTCGGAG
ATGGGCTGAACTGTGAGCCGGAGCAGCTGCCCATTGACCGCTGCTTACAGGACAATGGGCAGTGCCAT
GCAGACGCCAAATGTGTCGACCTCCACTTCCAGGATACCACTGTTGGGGTGTTCCATCTACGCTCCCC
ACTGGGCCAGTATAAGCTGACCTTTGACAAAGCCAGAGAGGCCTGTGCCAACGAAGCTGCGACCATGG
CAACCTACAACCAGCTCTCCTATGCCCAGAAGAGAGAAGAGAAATGAGTATGAAAGACCTGGGCACCT
ACAAGAAAGAGAGGACACTTTTGTTCACCCAGTGGCTCAATCAACCAGTCAACATCTAATGACCACCT
ACTGTGTGCCAGGCACAGAGGTGCAATAGGCAAAGCCAAGTACCACCTGTGCTCAGCAGGCTGGCTGC
AGACCGGGCGGGTTGCCTACCCCACAGCCTTCGCCT

| | | | |
|---|---|---|---|
| NOV9d, CG50736-02 | SEQ ID NO: 214 | 244 aa | MW at 26106.9kD |
| Protein Sequence | | | |

MCWPGRFGPDCLPCGCSDHGQCDDGITGSGQCLCETGWTGPSCDTQAVLSAVCTPPCSANATCKENNT
CECNLDYEGDGITCTVVDFCKQDNGGCAKVARCSQKGTKVSCSCQKGYKGDGHSCTEIDPCADGLNGG
CHEHATCKMTGPGKHKCECKSHYVGDGLNCEPEQLPIDRCLQDNGQCHADAKCVDLHFQDTTVGVFHL
RSPLGQYKLTFDKAREACANEAATMATYNQLSYAQKREEK

| | | |
|---|---|---|
| NOV9e, CG50736-03 | SEQ ID NO: 215 | 2512 bp |
| DNA Sequence | ORF Start: ATG at 416 | ORF Stop: TAA at 2423 |

ATAGGGCTCGAGCGGCTGCCCGGGCAGGTCTCATGCCTCAGCCTCCGGAGTAGTATTTTTAGTAGAGA
TGGTGTTTACCATGTGGGCCAGGCTGGTCTCGAACTCCTGGCCTCAAGTGATCCACCCGCCTCGGCCT
CCCAGAGTGCTGGGATTACAGGCATGAGCCACTGCACCCAGCCTTGTTTGTATTTTGAATTCCAAATG
GAAATACCTTCATGATCTTCCCACTACTAAAGGTTTAAATCTGGCACTGATACCTCTCCAAGAGGGCT
ATATACTATGCAGTGTTTCCCAGCATGTTTCACAAGAAAATTCTTTTTTGAGGATCATCTCACAGAAC
TTGGGATCTTTGCAACATGTATTGTGAAATCCAGGCCAGAGGAACCCCATGTTCCTTCCACACTGATA
TTCCACAATGGAGGCAAGAAAGGAGCTAGAGTCACTTCCTCCCTTTTGTCTGAACAGCCTCCACTCTA
TAATCCTGACCACAAAGCTTACTTCCCAGAGTCTGGGTGGGCCGAGAGGTGTGGAAGAGAGAATGGAG
GACAGGAGAGCCAAATGGCACATTGCAGCAAAAGACTCCTGCCTCTGGCTGAAACCCTCTGATCTTCT
GTTACAGGTTAAAGACTGGGACAAATACGGTTTAATGCCCCAGGTTCTTCGGTACCATGTGGTCGCCT
GCCACCAGCTGCTTCTGGAAAACCTGAAATTGATCTCAAATGCTACTTCCCTCCAAGGAGAGCCAATA
GTCATCTCCGTCTCTCAGAGCACGGTGTATATAAACAATAAGGCTAAGATCATATCCAGTGATATCAT
CAGTACTAATGGGATTGTTCATATCATAGACAAATTGCTATCTCCCAAAAATTTGCTTATCACTCCCA
AAGACAACTCTGGAAGAATTCTGCAAAATCTTACGACTTTGGCAACAAACAATGGCTACATCAAATTT
AGCAACTTAATACAGGACTCAGGTTTGCTGAGTGTCATCACCGATCCCATCCACACCCCAGTCACTCT
CTTCTGGCCCACCGACCAAGCCCTCCATGCCCTACCTGCTGAACAACAGGACTTCCTGTTCAACCAAG
ACAACAAGGACAAGCTGAAGGAGTATTTGAAGTTTCATGTGATACGAGATGCCAAGGTTTTAGCTGTG
GATCTTCCCACATCCACTGCCTGGAAGACCCTGCAAGGTTCAGAGCTGAGTGTGAAATGTGGACGTGG
CAGGGACATCGGTGACCTCTTTCTGAATGGCCAAACCTGCAGAATTGTGCAGCGCGAGCTCTTGTTTG
ACCTGGGTGTGGCCTACGGCATTGACTGTCTGCTGATTGATCCCACCCTGGGGGGCCGCTGTGACACC
TTTACTACTTTCGATGCCTCGGGGGAGTGTGGGAGCTGTGTCAATACTCCCAGCTGCCCAAGGTGGAG
TAAACCAAAGGGTGTGAAGCAGAAGTGTCTCTACAACCTGCCCTTCAAGAGGAACCTGGAAGGCTGCC
GGGAGCGGTGCAGCCTGGTGATACAGATCCCCAGGTGCTGCAAGGGCTACTTCGGGCGAGACTGTCAG
GCCTGCCCTGGAGGACCAGTTGCCCCGTGTAATAACCGGGGTGTCTGCCTTGATCAGTACTCGGCCAC
CGGAGAGTGTAAATGCAACACCGGCTTCAATGGGACGGCGTGTGAGATGTGCTGGCCGGGGAGATTTG
GGCCTGATTGTCTGCCCTGTGGCTGCTCAGACCACGGACAGTGCGATGATGGCATCACGGGCTCCGGG
CAGTGCCTCTGTGAAACGGGGTGGACAGGCCCCTCGTGTGACACTCAGGCAGTTTTGTCTGCAGTGTG
TACGCCTCCTTGTTCTGCTCATGCCACCTGTAAGGAGAACAACACGTGTGAGTGTAACCTGGATTATG
AAGGTGACGGAATCACATGCACAGTTGTGGATTTCTGCAAACAGGACAACGGGGGCTGTGCAAAGGTG
GCCAGATGCTCCCAGAAGGGCACGAAGGTCTCCTGCAGCTGCCAGAAGGGATACAAAGGGGACGGGCA
CAGCTGCACAGAGATAGACCCCTGTGCAGACGGCCTTAACGGAGGGTGTCACGAGCACGCCACCTGTA
AGATGACAGGCCCGGGCAAGCACAAGTGTGAGTGTAAAAGTCACTATGTCGGAGATGGGCTGAACTGT
GAGCCGGAGCAGCTGCCCATTGACCGCTGCTTACAGGACAATGGGCAGTGCCATGCAGACGCCAAATG
TGTCGACCTCCACTTCCAGGATACCACTGTTGGGGTGTTCCATCTACGCTCCCCACTGGGCCAGTATA
AGCTGACCTTTGACAAAGCCAGAGAGGCCTGTGCCAACGAAGCTGCGACCATGGCAACCTACAACCAG
CTCTCCTATGCCCAGAAGACCTGGTATTCCTTTACCAAGGAATAAAGCCTTTGATGCCAGGACCCAGA
CTCAAGGAGAATCTGAATCTCTGCTCTCCTGCTTGCTGGTCATGTGGCCTTGATATCAAGCCAC

| | | | |
|---|---|---|---|
| NOV9e, CG50736-03 | SEQ ID NO: 216 | 669 aa | MW at 73167.6kD |
| Protein Sequence | | | |

MEARKELESLPPFCLNSLHSIILTTKLTSQSLGGPRGVEERMEDRRAKWHIAAKDSCLWLKPSDLLLQ
VKDWDKYGLMPQVLRYHVVACHQLLLENLKLTSNATSLQGEPIVISVSQSTVYINNKAKIISSDIIST
NGIVHIIDKLLSPKNLLITPKDNSGRILQNLTTLATNNGYIKFSNLIQDSGLLSVITDPIHTPVTLFW
PTDQALHALPAEQQDFLFNQDNKDKLKEYLKFHVIRDAKVLAVDLPTSTAWKTLQGSELSVKCGAGRD
IGDLFLNGQTCRIVQRELLFDLGVAYGIDCLLIDPTLGGRCDTFTTFDASGECGSCVNTPSCPRWSKP
KGVKQKCLYNLPFKRNLEGCRERCSLVIQIPRCCKGYFGRDCQACPGGPVAPCNNRGVCLDQYSATGE
CKCNTGFNGTACEMCWPGRFGPDCLPCGCSDHGQCDDGITGSGQCLCETGWTGPSCDTQAVLSAVCTP
PCSAHATCKENNTCECNLDYEGDGITCTVVDFCKQDNGGCAKVARCSQKGTKVSCSCQKGYKGDGHSC
TEIDPCADGLNGGCHEHATCKMTGPGKHKCECKSHYVGDGLNCEPEQLPIDRCLQDNGQCHADAKCVD
LHFQDTTVGVFHLRSPLGQYKLTFDKEACANEAATMATYNQLSYAQKTWYSFTKE

| | | |
|---|---|---|
| NOV9f, CG50736-04 | SEQ ID NO: 217 | 2483 bp |
| DNA Sequence | ORF Start: ATG at 387 | ORF Stop: TAA at 2394 |

CTCATGCCTCAGCCTCCGGAGTAGTATTTTTAGTAGAGATGGTGTTTACCATGTGGGCCAGGCTGGTC
TCGAACTCCTGGCCTCAAGTGATCCACCCGCCTCGGCCTCCCAGAGTGCTGGGATTACAGGCATGAGC
CACTGCACCCAGCCTTGTTTGTATTTTGAATTCCAAATGGGAATTCCTTCATGATCTTCCCACTACTA
AAGGTTTAAATCTGGCACTGATACCTCTCCAAGAGGGCTATATACTATGCAGTGTTTCCCAGCATGTT
TCACAAGAAAATTCTTTTTTGAGGATCATCTCACAGAACTTGGGATCTTTGCAACATGTATTGTGAAA
TCCAGGCCAGAGGAACCCCATGTTCCTTCCACACTGATATTCCACAATGGAGGCAAGAAAGGAGCTAG
AGTCACTTCCTCCCTTTTGTCTGAACAGCCTCCACTCTATAATCCTGACCACAAAGCTTACTTCCCAG
AGTCTGGGTGGGCCGAGAGGTGTGGAAGAGAGAATGGAGGACAGGAGAGCCAAATGGCACATTGCAGC
AAAAGACTCCTGCCTCTGGCTGAAACCCTCTGATCTTCTGTTACAGGTTAAAGACTGGGACAAATACG
GTTTAATGCCCCAGGTTCTTCGGTACCATGTGGTCGCCTGCCACCAGCTGCTTCTGGAAAACCTGAAA
TTGATCTCAAATGCTACTTCCCTCCAAGGAGAGCCAATAGTCATCTCCGTCTCTCAGAGCACGGTGTA
TATAAACAATAAGGCTAAGATCATATCCAGTGATATCATCAGTACTAATGGGATTGTTCATATCATAG
ACAAATTGCTATCTCCCAAAAATTTGCTTATCACTCCCAAAGACAACTCTGGAAGAATTCTGCAAAAT
CTTACGACTTTGGCAACAAACAATGGCTACATCAAATTTAGCAACTTAATACAGGACTCAGGTTTGCT
GAGTGTCATCACCGATCCCATCCACACCCCAGTCACTCTCTTCTGGCCCACCGACCAAGCCCTCCATG
CCCTACCTGCTGAACAACAGGACTTCCTGTTCAACCAAGACAACAAGGACAAGCTGAAGGAGTATTTG
AAGTTTCATGTGATACGAGATGCCAAGGTTTTAGCTGTGGATCTTCCCACATCCACTGCCTGGAAGAC
CCTGCAAGGTTCAGAGCTGAGTGTGAAATGTGGAGCTGGCAGGGACATCGGTGACCTCTTTCTGAATG
GCCAAACCTGCAGAATTGTGCAGCGGGAGCTCTTGTTTGACCTGGGTGTGGCCTACGGCATTGACTGT
CTGCTGATTGATCCCACCCTGGGGGGCCGCTGTGACACCTTTACTACTTTCGATGCCTCGGGGGAGTG
TGGGAGCTGTGTCAATACTCCCAGCTGCCCAAGGTGGAGTAAAACCAAAGGGTGTGAGCAGAAGTGTC
TCTACAACCTGCCCTTCAAGAGGAACCTGGAAGGCTGCCGGGAGCGATGCAGCCTGGTGATACAGATC
CCCAGGTGCTGCAAGGGCTACTTCGGGCGAGACTGTCAGGCCTGCCCTGGAGGACCAGATGCCCCGTG
TAATAACCGGGGTGTCTGCCTTGATCAGTACTCGGCdACCGGAGAGTGTAAATGCAACACCGGCTTCA
ATGGGACGGCGTGTGAGATGTGCTGGCCGGGGAGATTTGGGCCTGATTGTCTGCCCTGTGGCTGCTCA
GACCACGGACAGTGCGATGATGGCATCACGGGCTCCGGGCAGTGCCTCTGTGAAACGGGGTGGACAGG
CCCCTCGTGTGACACTCAGGCAGTTTTGTCTGCAGTGTGTACGCCTCCTTGTTCTGCTCATGCCACCT
GTAAGGAGAACAACACGTGTGAGTGTAACCTGGATTATGAAGGTGACGGAATCACATGCACAGTTGTG
GATTTCTGCAAACAGGACAACGGGGGCTGTGCAAAGGTGGCCAGATGCTCCCAGAAGGGCACGAAGGT
CTCCTGCAGCTGCCAGAAGGGATACAAAGGGGACGGGCACAGCTGCACAGAGATAGACCCCTGTGCAG
ACGGCCTTAACGGAGGGTGTCACGAGCACGCCACCTGTAAGATGACAGGCCCGGGCAAGCACAAGTGT
GAGTGTAAAAGTCACTATGTCGGAGATGGGCTGAACTGTGAGCCGGAGCAGCTGCCCATTGACCGCTG
CTTACAGGACAATGGGCAGTGCCATGCAGACGCCAAATGTGTCGACCTCCACTTCCAGGATACCACTG
TTGGGGTGTTCCATCTACGCTCCCCACTGGGCCAGTATAAGCTGACCTTTGACAAAGCCAGAGAGGCC
TGTGCCAACGAAGCTGCGACCATGGCAACCTACAACCAGCTCTCCTATGCCCAGAAGACCTGGTATTC
CTTTACCAAGGAATAAAGCCTTTGATGCCAGGACCCAGACTCAAGGAGAATCTGAATCTCTGCTCTCC
TGCTTGCTGGTCATGTGGCCTTGATATCAAGCCAC

| | | | |
|---|---|---|---|
| NOV9f, CG50736-04 | SEQ ID NO: 218 | 669 aa | MW at 73183.5kD |
| Protein Sequence | | | |

MEARKELESLPPFCLNSLHSIILTTKLTSQSLGGPRGVEERMEDRPAKWHIAAKDSCLWLKPSDLLLQ
VKDWDKYGLMPQVLRYHVVACHQLLLENLKLISNATSLQGEPIVISVSQSTVYINNKAKIISSDIIST
NGIVHIIDKLLSPKNLLITPKDNSGRILQNLTTLATNNGYIKFSNLIQDSGLLSVITDPIHTPVTLFW
PTDQALHALPAEQQDFLFNQDNKDKLKEYLKFHVIRDAKVLAVDLPTSTAWKTLQGSELSVKCGAGRD
IGDLFLNGQTCRIVQRELLFDLGVAYGIDCLLIDPTLGGRCDTFTTFDASGECGSCVNTPSCPRWSKP
KGVKQKCLYNLPFKRNLEGCRERCSLVIQIPRCCKGYFGRDCQACPGGPDAPCNNRGVCLDQYSATGE
CKCNTGFNGTACEMCWPGRFGPDCLPCGCSDHGQCDDGITGSGQCLCETGWTGPSCDTQAVLSAVCTP
PCSAHATCKENNTCECNLDYEGDGITCTVVDFCKQDNGGCAKVARCSQKGTKVSCSCQKGYKGDGHSC
TEIDPCADGLNGGCHEHATCKMTGPGKHKCECKSHYVGDGLNCEPEQLPIDRCLQDNGQCHADAKCVD
LHFQDTTVGVFHLRSPLGQYKLTFDKAREACANEAATMATYNQLSYAQKTWYSFTKE

| | | |
|---|---|---|
| NOV9g, CG50736-05 | SEQ ID NO: 219 | 1353 bp |
| DNA Sequence | ORF Start: at 1 | ORF Stop: TAA at 1264 |

CTTCCCACATCCACTGCCTGGAAGACCCTGCAAGGTTCAGAGCTGAGTGTGAAATGTGGAGCTGGCAG
GGACATCGGTGACCTCTTTCTGAATGGCCAAACCTGCAGAATTGTGCAGCGGGAGCTCTTGTTTGACC
TGGGTGTGGCCTACGGCATTGACTGTCTGCTGATTGATCCCACCCTGGGGGGCCGCTGTGACACCTTT
ACTACTTTCGATGCCTCGGGGGAGTGTGGGAGCTGTGTCAATACTCCCAGCTGCCCAAGGTGGAGTAA
ACCAAAGGGTGTGAAGCAGAAGTGTCTCTACAACCTGCCCTTCAAGAGGAACCTGGAAGGCTGCCGGG
AGCGATGCAGCCTGGTGATACAGATCCCCAGGTGCTGCAAGGGCTACTTCGGGCGAGACTGTCAGGCC
TGCCCTGGAGGACCAGATGCCCCGTGTAATAACCGGGGTGTCTGCCTTGATCAGTACTCGGCCACCGG
AGAGTGTAAATGCAACACCGGCTTCAATGGGACGGCGTGTGAGATGTGCTGGCCGGGGAGATTTGGGC
CTGATTGTCTGCCCTGTGGCTGCTCAGACCACGGACAGTGCGATGATGGCATCACGGGCTCCGGGCAG
TGCCTCTGTGAAACGGGGTGGACAGGCCCCTCGTGTGACACTCAGGCAGTTTTGTCTGCAGTGTGTAC
GCCTCCTTGTTCTGCTCATGCCACCTGTAAGGAGAACAACACGTGTGAGTGTAACCTGGATTATGAAG
GTGACGGAATCACATGCACAGTTGTGGATTTCTGCAAACAGGACAACGGGGGCTGTGCAAAGGTGGCC
AGATGCTCCCAGAAGGGCACGAAGGTCTCCTGCAGCTGCCAGAAGGGATACAAAGGGGACGGGCACAG
CTGCACAGAGATAGACCCCTGTGCAGACGGCCTTAACGGAGGGTGTCACGAGCACGCCACCTGTAAGA
TGACAGGCCCGGGCAAGCACAAGTGTGAGTGTAAAAGTCACTATGTCGGAGATGGGCTGAACTGTGAG
CCGGAGCAGCTGCCCATTGACCGCTGCTTACAGGACAATGGGCAGTGCCATGCAGACGCCAAATGTGT
CGACCTCCACTTCCAGGATACCACTGTTGGGGTGTTCCATCTACGCTCCCCACTGGGCCAGTATAAGC
TGACCTTTGACAAAGCCAGAGAGGCCTGTGCCAACGAAGCTGCGACCATGGCAACCTACAACCAGCTC
TCCTATGCCCAGAAGACCTGGTATTCCTTTACCAAGGAATAAAGCCTTTGATGCCAGGACCCAGACTC
AAGGAGAATCTGAATCTCTGCTCTCCTGCTTGCTGGTCATGTGGCCTTGATATCAAGCCAC

| | | | |
|---|---|---|---|
| NOV9g, CG50736-05 | SEQ ID NO: 220 | 421 aa | MW at 45276.6kD |
| Protein Sequence | | | |

LPTSTAWKTLQGSELSVKCGAGRDIGDLFLNGQTCRIVQRELLFDLGVAYGIDCLLIDPTLGGRCDTF
TTFDASGECGSCVNTPSCPRWSKPKGVKQKCLYNLPFKRNLEGCRERCSLVIQIPRCCKGYFGRDCQA
CPGGPDAPCNNRGVCLDQYSATGECKCNTGFNGTACEMCWPGRFGPDCLPCGCSDHGQCDDGITGSGQ
CLCETGWTGPSCDTQAVLSAVCTPPCSAHATCKENNTCECNLDYEGDGITCTVVDFCKQDNGGCAKVA
RCSQKGTKVSCSCQKGYKGDGHSCTEIDPCADGLNGGCHEHATCKMTGPGKHKCECKSHYVGDGLNCE
PEQLPIDRCLQDNGQCHADAKCVDLHFQDTTVGVFHLRSPLGQYKLTFDKAREACANEAATMATYNQL
SYAQKTWYSFTKE

| | | |
|---|---|---|
| NOV9h, CG50736-06 | SEQ ID NO: 221 | 3625 bp |
| DNA Sequence | ORF Start: ATG at 387 | ORF Stop: TAG at 2022 |

CTCATGCCTCAGCCTCCGGAGTAGTATTTTTAGTAGAGATGGTGTTTACCATGTGGGCCAGGCTGGTC
TCGAACTCCTGGCCTCAAGTGATCCACCCGCCTCGGCCTCCCAGACTGCTGGGATTACAGGCATGAGC
CACTGCACCCAGCCTTGTTTGTATTTTGAATTCCAAATGGAAATACCTTCATGATCTTCCCACTACTA
AAGGTTTAAATCTGGCACTGATACCTCTCCAAGAGGGCTATATACTATGCAGTGTTTCCCAGCATGTT
TCACAAGAAAATTCTTTTTTGAGGATCATCTCACAGAACTTGGGATCTTTGCAACATGTATTGTGAAA
TCCAGGCCAGAGGAACCCCATGTTCCTTCCACACTGATATTCCACAATGGAGGCAAGAAAGGAGCTAG
AGTCACTTCCTCCCTTTTGTCTGAACAGCCTCCACTCTATAATCCTGACCACAAAGCTTACTTCCCAG
AGTCTGGGTGGGCCGAGAGGTGTGGAAGAGAGAATGGAGGACAGGAGAGCCAAATGGCACATTGCAGC
AAAAGACTCCTGCCTCTGGCTGAAACCCTCTGATCTTCTGTTACAGGTTAAAGACTGGGACAAATACG
GTTTAATGCCCCAGGTTCTTCGGTACCATGTGGTCGCCTGCCACCAGCTGCTTCTGGAAAACCTGAAA
TTGATCTCAAATGCTACTTCCCTCCAAGGAGAGCCAATAGTCATCTCCGTCTCTCAGAGCACGGTGTA
TATAAATAATAAGGCTAAGATCATATCCAGTGATATCATCAGTACTAATGGGATTGTTCATATCATAG
ACAAATTGCTATCTCCCAAAAATTTGCTTATCACTCCCAAAGACAACTCTGGAAGAATTCTGCAAAAT
CTTACGACTTTGGCAACAAACAATGGCTACATCAAATTTAGCAACTTAATACAGGACTCAGGTTTGCT
GAGTGTCATCACCGATCCCATCCACACCCCAGTCACTCTCTTCTGGCCCACCGACCAAGCCCTCCATG
CCCTACCTGCTGAACAACAGGACTTCCTGTTCAACCAAGACAACAAGGACAAGCTGAAGGAGTATTTG
AAGTTTCATGTGATACGAGATGCCAAGGTTTTAGCTGTGGATCTTCCCACATCCACTGCCTGGAAGAC
CCTGCAAGGTTCAGAGCTGAGTGTGAAATGTGGAGCTGGCAGGGACATCGGTGACCTCTTTCTGAATG
GCCAAACCTGCAGAATTGTGCAGCGGGAGCTCTTGTTTGACCTGGGTGTGGCCTACGGCATTGACTGT
CTGCTGATTGATCCCACCCTGGGGGGCCGCTGTGACACCTTTACTACTTTCGATGCCTCGGGGGAGTG
TGGGAGCTGTGTCAATACTCCCAGCTGCCCAAGGTGGAGTAAACCAAAGGGTGTGAAGCAGAAGTGTC
TCTACAACCTGCCCTTCAAGAGGAACCTGQAAGGCTGCCGGGAGCGGTGCAGCCTGGTGATACAGATC
CCCAGGTGCTGCAAGGGCTACTTCGGGCGAGACTGTCAGGGTGAGGGTGCCTCTTCCCCCCTCGCAAC
TCTAAAAGTGTCTGCCTTGATCAGTACTCGGCCACCGGAGAGTGTAAATGCAACACCGGCTTCAATGG
GACGGCGTGTGAGATGTGCTGGCCGGGGAGATTTGGGCCTGATTGTCTGCCCTGTGGCTGCTCAGACC
ACGGACAGTGCGATGATGGCATCACGGGCTCCGGGCAGTGCCTCTGTGAAACGGGGTGGACAGGCCCC
TCGTGTGACACTCAGGCAGTTTTGCCTGCAGTGTGTACGCCTCCTTGTTCTGCTCATGCCACCTGTAA
GGAGAACAACACGTGTGAGTGTAACCTGGATTATGAAGGTGACGGAATCACATGCACAGTTGTGGATT
TCTGCAAACAGGACAACGGGGGCTGTGCAAAGGTGGCCAGATGCTCCCAGAAGGGCACGAAGGTCTCC
TGCAGCTGCCAGAAGGGATACAAAGGGGACGGGCACAGCTGCACAGAGATAGACCCCTGTGCAGACGG
CCTTAACGGAGGGTGTCACGAGCACGCCACCTGTAAGATGACAGGCCCGGGCAAGCACAAGTGTGAGT
GTAAAAGTCACTATGTCGGAGATGGGCTGAACTGTGAGCCGGAGCAGCTGCCCATTGACCGCTGCTTA
CAGGACAATGGGCAGTGCCATGCAGACGCCAAATGTGTCGACCTCCACTTCCAGGATACCACTGTTGG
GGTGTTCCATCTACGCTCCCCACTGGGCCAGTATAAGCTGACCTTTGACAAAGCCAGAGAGGCCTGTG
CCAACGAAGCTGCGACCATGGCAACCTACAACCAGCTCTCCTATGCCCAGAAGGCCAAGTACCACCTG
TGCTCAGCAGGCTGGCTGGAGACCGGGCGGGTTGCCTACCCCACAGCCTTCGCCTCCCAGAACTGTGG
CTCTGGTGTGGTTGGGATAGTGGACTATGGACCTAGACCCAACAAGAGTGAAATGTGGGATGTCTTCT
GCTATCGGATGAAAGGAAGTGCTGGCCTATTCCAACAGCTCAGCTCGAGGCCGTGCATTTCTAGAACA
CCTGACTGACCTGTCCATCCGCGGCACCCTCTTTGTGCCACAGAACAGTGGGCTGGGGGAGAATGAGA
CCTTGTCTGGGCGGGACATCGAGCACCACCTCGCCAATGTCAGCATGTTTTTCTACAATGACCTTGTC
AATGGCACCACCCTGCAAACGAGGCTGGGAAGCAAGCTGCTCATCACTGCCAGCCAGGACCCACTCCA
ACCGACGGAGACCAGGTTTGTTGATGGAAGAGCCATTCTGCAGTGGGACATCTTTGCCTCCAATGGGA
TCATTCATGTCATTTCCAGGCCTTTAAAAGCACCCCCTGCCCCCGTGACCTTGACCCACACTGGCTTG
GGAGCAGGGATCTTCTTTGCCATCATCCTGGTGACTGGGGCTGTTGCCTTGGCTGCTTACTCCTACTT
TCGGCAAGCAGCAGCCTGAGAATATCTCGAACCCCTTGTATGAGAGCACAACCTCAGCTCCCCCAGAA
TTGGCAAGCAGCAGCCTGAGAATATCTCGAACCCCTTGTATGAGAGCACAACCTCAGCTCCCCCAGAA
CCTTCCTACGACCCCTTCACGGACTCTGAAGAACGGCAGCTTGAGGGCAATGACCCCTTGAGGACACT
GTGAGGGCCTGGACGGGAGATGCCAGCCATCACTCACTGCCACCTGGGCCATCAACTGTGAATTCTCA
GCACCAGTTGCCTTTTAGGAACGTAAAGTCCTTTAAGCACTCAGAAGCCATACCTCATCTCTCTGGCT
GATCTGGGGGTTGTTTCTGTGGGTGAGAGATGTGTTGCTGTGCCCACCCAGTACAGCTTCCTCCTCTG
ACCCTTTGGCTCTTCTTCCTTTGTACTCTTCAGCTGGCACCTGCTCCATTCTGCCCTACATGATGGGT
AACTGTGATCTTTCTTCCCTGTTAGATTGTAAGCCTCCGTCTTTGTATCCCAGCCCCTAGCCCAGTGC
CTGACACAGGAACTGTGCACAATAAAGGTTTATGGAACAGAAACAAAGTCAAAAAAAAAAAAAAAAAA
AAAAAAAAAAAAAAAAAAAAC

| | | | |
|---|---|---|---|
| NOV9h, CG50736-06 | SEQ ID NO: 222 | 545 aa | MW at 60038.8kD |
| Protein Sequence | | | |

MEARKELESLPPFCLNSLHSIILTTKLTSQSLGGPRGVEERMEDRRAKWHIAAKDSCLWLKPSDLLLQ
VKDWDKYGLMPQVLRYHVVACHQLLLENLKLISNATSLQGEPIVISVSQSTVYINNKAKIISSDIIST
NGIVHIIDKLLSPKNLLITPKDNSGRILQNLTTLATNNGYIKFSNLIQDSGLLSVITDPIHTPVTLFW
PTDQALHALPAEQQDFLFNQDNKDKLKEYLKFHVIRDAKVLAVDLPTSTAWKTLQGSELSVKCGAGRD
IGDLFLNGQTCRIVQRELLFDLGVAYGIDCLLIDPTLGGRCDTFTTFDASGECGSCVNTPSCPRWSKP
KGVKQKCLYNLPFKRNLEGCRERCSLVIQIPRCCKGYFGRDCQGEGASSPLATLKVSALISTRPPESV
NATPASMGRRVRCAGRGDLGLIVCPVAAQTTDSAMMASRAPGSASVKRGGQAPRVTLRQFCLQCVRLL
VLLMPPVRRTTRVSVTWIMKVTESHAQLWISANRTTGAVQRWPDAPRRARRSPAAARRDTKGTGTAAQ
R

| | | |
|---|---|---|
| NOV9i, CG50736-07 | SEQ ID NO: 223 | 1624 bp |
| DNA Sequence | ORF Start: ATG at 387 | ORF Stop: TAA at 1530 |

CTCATGCCTCAGCCTCCGGAGTAGTATTTTTAGTAGAGATGGTGTTTACCATGTGGGCCAGGCTGGTC
TCGAACTCCTGGCCTCAAGTGATCCACCCGCCTCGGCCTCCCAGAGTGCTGGGATTACAGGCATGAGC
CACTGCACCCAGCCTTGTTTGTATTTTGAATTCCAAATGGAAATACCTTCATGATCTTCCCACTACTA
AAGGTTTAAATCTGGCACTGATACCTCTCCAAGAGGGCTATATACTATGCAGTGTTTCCCAGCATGTT
TCACAAGAAAATTCTTTTTTGAGGATCATCTCACAGAACTTGGGATCTTTGCAACATGTATTGTGAAA
TCCAGGCCAGAGGAACCCCATGTTCCTTCCACACTGATATTCCACAATGGAGGCAAGAAAGGAGCTAG
AGTCACTTCCTCCCTTTTGTCTGAACAGCCTCCACTCTATAATCCTGACCACAAAGCTTACTTCCCAG
AGTCTGGGTGGGCCGAGAGGTGTGGAAGAGAGAATGGAGGACAGGAGAGCCAAATGGCACATTGCAGC
AAAAGACTCCTGCCTCTGGCTGAAACCCTCTGATCTTCTGTTACAGGTTAAAGACTGGGACAAATACG
GTTTAATGCCCCAGGTTCTTCGGTACCATGTGGTCGCCTGCCACCAGCTGCTTCTGGAAAACCTGAAA
TTGATCTCAAATGCTACTTCCCTCCAAGGAGAGCCAATAGTCATCTCCGTCTCTCAGAGCACGGTGTA
TATAAACAATAAGGCTAAGATCATATCCAGTGATATCATCAGTACTAATGGGATTGTTCATATCATAG
ACAAATTGCTATCTCCCAAAAATTTGCTTATCACTCCCAAAGACAACTCTGGAAGAATTCTGCAAAAT
CTTACGACTTTGGCAACAAACAATGGCTACATCAAATTTAGCAACTTAATACAGGACTCAGGTTTGCT
GAGTGTCATCACCGATCCCATCCACACCCCAGTCACTCTCTTCTGGCCCACCGACCAAGCCCTCCATG
CCCTACCTGCTGAACAACAGGACTTCCTGTTCAACCAAGACAACAAGGACAAGCTGAAGGAGTATTTG
AAGTTTCATGTGATACGAGATGCCAAGGTTTTAGCTGTGGATCTTCCCACATCCACTGCCTGGAAGAC
CCTGCAAGGTTCAGAGCTGAGTGTGAAATGTGGAGCTGGCAGGGACATCGGTGACCTCTTTCTGAATG
GCCAAACCTGCAGAATTGTGCAGCGGGAGCTCTTGTTTGACCTGGGTGTGGCCTACGGCATTGACTGT
CTGCTGATTGATCCCACCCTGGGGGGCCGCTGTGACACCTTTACTACTTTCGATGCCTCGGGGGAGTG
TGGGAGCTGTGTCAATACTCCCAGCTGCCCAAGGTGGAGTAAACCAAAGGGTGTGAAGCAGAAGTGTC
TCTACAACCTGCCCTTCAAGAGGAACCTGGAAGGCTGCCGGGAGCGGTGCAGCCTGGTGATACAGATC
CCCAGCCTGCCCTGGAGGACCAGATGCCCCGTGTAATAACCGGGGTGTCTGCCTTGATCAGTACTCGG
CCACCGGAGAGTGTAAATGCAACACCGGCTTCAATGGGACGGCGTGTGAGATGTGCTGGC

| | | | |
|---|---|---|---|
| NOV9i, CG50736-07 | SEQ ID NO: 224 | 381 aa | MW at 42565.8kD |
| Protein Sequence | | | |

MEARKELESLPPFCLNSLHSIILTTKLTSQSLGGPRGVEERMEDRRAKWHIAAKDSCLWLKPSDLLLQ
VKDWDKYGLMPQVLRYHVVACHQLLLENLKLISNATSLQGEPIVISVSQSTVYINNKAKIISSDIIST
NGIVHIIDKLLSPKNLLITPKDNSGRILQNLTTLATNNGYIKFSNLIQDSGLLSVITDPIHTPVTLFW
PTDQALHALPAEQQDFLFNQDNKDKLKEYLKFHVIRDAKVLAVDLPTSTAWKTLQGSELSVKCGAGRD
IGDLFLNGQTCRIVQRELLFDLGVAYGIDCLLIDPTLGGRCDTFTTFDASGECGSCVNTPSCPRWSKP
KGVKQKCLYNLPFKRNLEGCRERCSLVIQIPSLPWRTRCPV

| | | |
|---|---|---|
| NOV9j, CG50736-08 | SEQ ID NO: 225 | 1857 bp |
| DNA Sequence | ORF Start: at 1 | ORF Stop: TAA at 1768 |

GTTCTTCGGTACCATGTGGTCGCCTGCCACCAGCTGCTTCTGGAAAACCTGAAATTGATCTCAAATGC
TACTTCCCTCCAAGGAGAGCCAATAGTCATCTCCGTCTCTCAGAGCACGGTGTATATAAACAATAAGG
CTAAGATCATATCCAGTGATATCATCAGTACTAATGGGATTGTTCATATCATAGACAAATTGCTATCT
CCCAAAAATTTGCTTATCACTCCCAAAGACAACTCTGGAAGAATTCTGCAAAATCTTACGACTTTGGC
AACAAACAATGGCTACATCAAATTTAGCAACTTAATACAGGACTCAGGTTTGCTGAGTGTCATCACCG
ATCCCATCCACACCCCAGTCACTCTCTTCTGGCCCACCGACCAAGCCCTCCATGCCCTACCTGCTGAA
CAACAGGACTTCCTGTTCAACCAAGACAACAAGGACAAGCTGAAGGAGTATTTGAAGTTTCATGTGAT
ACGAGATGCCAAGGTTTTAGCTGTGGATCTTCCCACATCCACTGCCTGGAAGACCCTGCAAGGTTCAG
AGCTGAGTGTGAAATGTGGAGCTGGCAGGGACATCGGTGACCTCTTTCTGAATGGCCAAACCTGCAGA
ATTGTGCAGCGGGAGCTCTTGTTTGACCTGGGTGTGGCCTACGGCATTGACTGTCTGCTGATTGATCC
CACCCTGGGGGGCCGCTGTGACACCTTTACTACTTTCGATGCCTCGGGGGAGTGTGGGAGCTGTGTCA
ATACTCCCAGCTGCCCAAGGTGGAGTAAACCAAAGGGTGTGAAGCAGAAGTGTCTCTACAACCTGCCC
TTCAAGAGGAACCTGGAAGGCTGCCGGGAGCGATGCAGCCTGGTGATACAGATCCCCAGGTGCTGCAA
GGGCTACTTCGGGCGAGACTGTCAGGCCTGCCCTGGAGGACCAGATGCCCCGTGTAATAACCGGGGTG
TCTGCCTTGATCAGTACTCGGCCACCGGAGAGTGTAAATGCAACACCGGCTTCAATGGGACGGCGTGT
GAGATGTGCTGGCCGGGGAGATTTGGGCCTGATTGTCTGCCCTGTGGCTGCTCAGACCACGGACAGTG
CGATGATGGCATCACGGGCTCCGGGCAGTGCCTCTGTGAAACGGGGTGGACAGGCCCCTCGTGTGACA
CTCAGGCAGTTTTGTCTGCAGTGTGTACGCCTCCTTGTTCTGCTCATGCCACCTGTAAGGAGAACAAC
ACGTGTGAGTGTAACCTGGATTATGAAGGTGACGGAATCACATGCACAGTTGTGGATTTCTGCAAACA
GGACAACGGGGGCTGTGCAAAGGTGGCCAGATGCTCCCAGAAGGGCACGAAGGTCTCCTGCAGCTGCC
AGAAGGGATACAAAGGGGACGGGCACAGCTGCACAGAGATAGACCCCTGTGCAGACGGCCTTAACGGA
GGGTGTCACGAGCACGCCACCTGTAAGATGACAGGCCCGGGCAAGCACAAGTGTGAGTGTAAAAGTCA
CTATGTCGGAGATGGGCTGAACTGTGAGCCGGAGCAGCTGCCCATTGACCGCTGCTTACAGGACAATG
GGCAGTGCCATGCAGACGCCAAATGTGTCGACCTCCACTTCCAGGATACCACTGTTGGGGTGTTCCAT
CTACGCTCCCCACTGGGCCAGTATAAGCTGACCTTTGACAAAGCCAGAGAGGCCTGTGCCAACGAAGC
TGCGACCATGGCAACCTACAACCAGCTCTCCTATGCCCAGAAGACCTGGTATTCCTTTACCAAGGAAT
AAAGCCTTTGATGCCAGGACCCAGACTCAAGGAGAATCTGAATCTCTGCTCTCCTGCTTGCTGGTCAT
GTGGCCTTGATATCAAGCCAC

| | | | |
|---|---|---|---|
| NOV9j, CG50736-08 | SEQ ID NO: 226 | 589 aa | MW at 63994.0kD |
| Protein Sequence | | | |

VLRYHVVACHQLLLENLKLISNATSLQGEPIVISVSQSTVYINNKAKIISSDIISTNGIVHIIDKLLS
PKNLLITPKDNSGRILQNLTTLATNNGYIKFSNLIQDSGLLSVITDPIHTPVTLFWPTDQALHALPAE
QQDFLFNQDNKDKLKEYLKFHVIRDAKVLAVDLPTSTAWKTLQGSELSVKCGAGRDIGDLFLNGQTCR
IVQRELLFDLGVAYGIDCLLIDPTLGGRCDTFTTFDASGECGSCVNTPSCPRWSKPKGVKQKCLYNLP
FKRNLEGCRERCSLVIQIPRCCKGYFGRDCQACPGGPDAPCNNRGVCLDQYSATGECKCNTGFNGTAC
EMCWPGRFGPDCLPCGCSDHGQCDDGITGSGQCLCETGWTGPSCDTQAVLSAVCTPPCSAHATCKENN
TCECNLDYEGDGITCTVVDFCKQDNGGCAKVARCSQKGTKVSCSCQKGYKGDGHSCTEIDPCADGLNG
GCHEHATCKMTGPGKHKCECKSHYVGDGLNCEPEQLPIDRCLQDNGQCHADAKCVDLHFQDTTVGVFH
LRSPLGQYKLTFDKAREACANEAATMATYNQLSYAQKTWYSFTKE

| | | |
|---|---|---|
| NOV9k, CG50736-10 | SEQ ID NO: 227 | 8495 bp |
| DNA Sequence | ORF Start: ATG at 210 | ORF Stop: TGA at 7461 |

AATCATCCCACATGCTAAGAATCTAAGATGTATAAAATAAAGTGGTGAAAGATGAAAATGAAATTTTA
TCAAGGTTAGAGTCAGGTTGGAGTGGCCATTGTTTACCAAACTGAGAAATCTAAATTTTATTTGGTTG
GTAATTGAGAGTCATTGAGATATTTTGGGGAAGGTCACCCTGATGCCTTTGCTAATCAAATGAAATGA
ATGAAATGGAGAGGCAAGAAACTGGAAATAGCAAGACGAGGTATCATGCTACTGCAATAGTCCAGGCA
AAACATGATAAAGGCCTCAACAAGAATGGCACCAGTGGAGATGAAGAGCAGAAGATCAAGGTGGGAGA
CAGAGACAGAGAAAACAAAGGATTTGATGGCTTATTAGATGTTTGGAATACTTTAAACTTTATTCATC
CTTGCTTTGCTGTGTGCAACTGTGTGCATGGGGTGTGCAACAGTGGACTAGATGGCGATGGAACCTGT
GAGTGCTACTCTGCGTACACTGGCCCCAAGTGTGACAAGCTCACAGAAAACTTTCACACCTCTCATCT
GACACTGTGGCCTGTGCACGACTCCAAGCACTGGGGAAGCCTTCGACATCAGAATATGAATGGCACCT
GTTCTTCCGGGGGCGGCAAGGGGGATCCCGATGTTTATCAAAATGGGTTGATTTTCCACGGAGGGGGT
ACTTCTGGAGGTCTATCGTCATCACGAAACAGACGAAGTAGTGTCAAGCGTCCTGAGAAGTGGAAGGG
GGACGATCGAGATGGAGGTGGCAAGGAAGGCCAGCAGCGGCGGCGGGCAGACACAGAGTCGAGTCTTC
AAAGAGGTCACATCAAAACGCCCCTGCCCCACAGGCAAGGTGAAGCGCGGATCACGGAGACAACGGGG
AATTGTGTTTCTGCTGGCATGACTGGAACCAATGCCAATCACACAAAAGTTCACCCTACGGTTCAGTC
CTTGACAGAATATGATTCCTTTCAGACTCATTCCACCAGCAGACTGAAGGAATTTGAGAAACAGCAGG
TGAAGGAAAGATTTTCTGACCCTCCCCTAATGCAGGCTATAAAACCCTCACATGAGAAGTACCCTCCT
TATGCCCAGAGAAAAGGAACATCTTTGTCTCCAAAGACACAGGGACACGGAGATGATGAACAGGCCTT
GCTAAGTTTCCTCCACTCTATTACCCTTAGCTTGTACCTTTATCCAACCACATTCTTCCATGACTCTC
CAGTCTTCATCAAACCTGGCATAAAAACACTCAGACTTAACCACTTCTTTGGGTCTTCATTTCCTTAT
GAAGGCTCCAGTGTCATANNNNNNATGGGAATTGAGGTTTGGAAAAACTGGTGCCAAAATGCTGATAC
CCTGGCTGCTGCCCCTGCTCCATCCCTGAATGTGCAGCCTTGCTCTGCCCAGAAAATTCCAGATGTTC
GCCTTCCACTGAAGATGAAAACAAACTGGAATGCAAATGCCTTCCCAATTACCGAGGCGATGGCAAAT
ACTGCGACCCCATCAATCCATGTTTACGAAAAATCTGCCACCCTCATGCTCATTGTACGTACCTGGGA
CCAAATCGGCACAGTTGTACATGCCAAGAAGGCTACCGTGGGGATGGCCAAGTGTGCTTGCCTGTGGA
CCCCTGCCAAATTAACTTTGGAAACTGCCCTACAAAGTCTACAGTGTGCAAATATGATGGGCCTGGAC
AGATGCATTTGCCAGAAAGGTTACGTGGGTGATGGCTTAACGTGTTATGGAAACATTATGGAGCGACT
CAGAGAATTAAATACTGAACCCAGAGGAAAATGGCAAGGAAGGCTGACCTCTTTCATCTCACTCCTAG
AAAGTATACAAATTGTAAGTGTACAACTCAGTGAATTTTCCCAACGTGAACCTACTTGTGTAAACACC
AAGTCCATTGCCAGCAACCTAGAAGGCCCCCTGGTCCCCCTTTCCAATCATTACCCTCTACAGGTAAA
TGAGCTTTTGGTGGATAATAAAGCTGCTCAATACTTTGTGAAACTCCACATAATTGCTGGTCAGATGA
ACATCGAATATATGAATAACACAGACATGTTCTACACCTTGACTGGAAAGTCGGGGGAAATCTTCAAC
AGCGATAAGGACAATCAAATAAAGCTTAAACTCCATGGAGGCAAAAAGAAGGTAAAAATTATACAAGG
GGACATCATTGCTTCCAATGGGCTTCTGCACATCCTTGACAGAGCCATGGACAAGTTAGAACCCACAT
TTGAGAGCAACAATGAGGALACCAATTTGGGACATGCCTTAGATGAGGATGGAGTTGGTGGACCATAC
ACCATTTTTGTTCCAAATAATGAAGCATTGAATAACATGAAGGACGGCACTCTCGATTACCTCCTTTC
TCCAGAGCTTGAAGTGGCCACTCTCATCTCCACCCCTCACATCAGGAGCATGGCCAACCAGCTCATAC
AGTTCAACACCACCGACAATGGACAGATTCTGGCAAATGATGTGGCAATGGAAGAAATTGAGATCACT
GCCAAAAATGGCCGAATTTACACACTGACAGGAGTTCTCATTCCTCCCTCCATTGTCCCGATTCTGCC
CCATCGATGTGATGAAACAAAGAGAGAGATGAAACTGGGCACTTGTGTGAGCTGTTCTCTGGTGTACT
GGAGCAGATGTCCTGCTAACTCTGAGCCCACAGCACTCTTCACACACAGATGTGTCTACAGTGGCAGG
TTTGGGAGCCTGAAGAGCGGCTGTGCCCGGTACTGCAATGCCACTGTGAAGTGTGCAGATAGCCTCGG
CGGCAACGGGACATGCATTTGTGAGGAGGGCTTCCAAGGCTCCCAGTGTCAGTTCTGCTCTGATCCCA
ATAAATACGGACCTCGGTGTAACAAAAAATGCCTGTGCGTTCACGGAACATGCAATAACAGGATAGAC
AGCGATGGGGCCTGCCTCACTGGCACATGCAGAGACGGCTCTGCCGGGAGACTCTGTGATAAGCAGAC
CTCAGCCTGTGGGCCCTACGTGCAGTTCTGTCACATCCACGCCACCTGTGAATACAGCAATGGGACAG
CCAGTTGTATTTGCAAAGCAGGATATGAAGGAGATGGAACTCTGTGTTCTGAGATGGACCCTTGCACA
GGACTAACTCCAGGAGGCTGTAGCCGCAATGCAGAATGCATCAAAACTGGCACGGGCACCCACACCTG
CGTGTGTCAGCAGGGTTGGACAGGGAATGGGAGAGACTGCTCGGAGATCAACAACTGCCTGCTGCCCA
GTGCAGGCGGCTGCCACGACAACGCATCCTGTTTGTATGTGGGTCCCGGGCAGAATGAGTGTGAGTGC
AAGAAAGGATTTCGAGGAAATGGGATTGACTGTGAACCAATAACTTCATGCTTGGAACAAACCGGGAA
ATGTCATCCATTGGCAAGCTGTCAATCTACTTCGTCTGGTGTCTGGAGCTGTGTTTGTCAAGAGGGCT
ATGAAGGAGATGGCTTTCTGTGCTATGGAAACGCAGCAGTGGAATTGTCATTTCTCTCCGAAGCAGCT
ATATTTAACCGATGGATAAATAATGCTTCTCTACAACCCACACTGTCAGCCACCTCAAACCTCACTGT
CCTCGTGCCTTCCCAACAAGCTACTGAGGACATGGACCAGGATGAGAAAAGCTTCTGGTTGTCACAGA
GCAATATTCCAGCCCTAATAAAGTACCATATGCTACTAGGCACATACAGAGTGGCAGATCTGCAGACC
CTGTCTTCTTCTGACATGTTGGCAACATCTTTGCAGGGCAACTTCCTTCACTTGGCAAAGGTGGATGG
GAATATCACAATTGAAGGGGCCTCCATTGTCGATGGGGACAACGCAGCCACAAATGGAGTGATACACA
TCATCAACAAGGTGCTGGTCCCACAAAGACGTCTAACTGGCTCCTTACCAAACCTGCTCATGCGGCTG
GAACAGATGCCTGACTATTCCATCTTCCGGGGCTACATCATTCAATATAATCTGGCGAATGCAATTGA
GGCTGCCGATGCCTACACAGTGTTTGCTCCAAACAACAATGCCATCGAGAATTACATCAGGGAGAAGA
AAGTCTTGTCTCTAGAGGAGGACGTCCTCCGGTATCATGTGGTCCTGGAGGAGAAACTCCTGAAGAAT
GACCTGCACAATGGCATGCATCGTGAGACCATGCTGGGTTTCTCCTATTTCCTTAGCTTCTTTCTCCA
TAATGACCAGCTCTATGTAAATGAGGCTCCAATAAACTACACCAATGTAGCCACTGATAAGGGAGTGA
TCCATGGTTTGGGAAAAGTTCTGGAAATTCAGAAGAACAGATGTGATAATAATGACACTACTATTATA
CGAGGAAGATGTAGGACATGCTCCTCAGAGCTGACCTGCCCATTCGGAACTAAATCTCTAGGTAATGA
GAAGAGGAGATGCATCTATACCTCCTATTTCATGGGAAGACGAACCCTGTTTATTGGGTGCCAGCCAA
AATGTGTGAGAACCGTCATTACGAGAGAATGCTGTGCCGGCTTCTTTGGCCCCCAATGCCAGCCCTGC
CCAGGGAATGCCCAGAATGTCTGCTTTGGTAATGGCATCTGTTTGGATGGAGTGAATGGCACAGGTGT
GTGTGAGTGTGGGGAGGGCTTCAGCGGCACAGCCTGCGAGACCTGCACCGAGGGCAAGTACGGCATCC
ACTGTGACCAAGCATGTTCTTGTGTCCATGGGAGATGCAACCAAGGACCCTTGGGAGATGGCTCCTGT
GACTGTGATGTTGGCTGGCGAGGAGTGCATTGTGACAATGCAACCACAGAAGACAACTGCAATGGGAC
ATGCCATACCAGCGCCAACTGCCTCACCAACTCAGATGGTACAGCTTCATGCAAGTGTGCAGCAGGAT
TCCAAGGAAACGGGACCATCTGCACAGCAATCAATGCCTGTGAGATCAGCAATGGAGGTTGCTCTGCC
AAGGCTGACTGTAAGAGAACCACCCCAGGAAGGCGAGTGTGCACGTGCAAAGCAGGCTACACGGGTGA
TGGCATTGTGTGCCTGGAAATCAACCCGTGTTTGGAGAACCATGGTGGCTGTGACAAGAATGCGGAGT
GCACACAGACAGGACCCAACCAGGCTGCCTGTAACTGTTTGCCAGCATACACTGGAGATGGAAAGGTC
TGCACACTCATCAATGTCTGCTTAACTAAAAATGGCGGCTGTAGTGAATTTGCCATCTGCAACCACAC
TGGGCAAGTAGAAAGGACTTGTACTTGCAAGCCAAACTACATTGGAGATGGATTTACCTGCCGCGGCA
GCATTTATCAGGAGCTTCCCAAGAACCCGAAAACTTCCCAGTATTTCTTCCAGTTGCAGGAGCATTTC
GTGAAAGATCTGGTCGGCCCAGGCCCCTTCACTGTTTTTGCACCTTTATCTGCAGCCTTTGATGAGGA
AGCTCGGGTTAAAGACTGGGACAAATACGGTTTAATGCCCCAGGTTCTTCGGTACCATGTGGTCGCCT
GCCACCAGCTGCTTCTGGAAAACCTGAAATTGATCTCAAATGCTACTTCCCTCCAAGGAGAGCCAATA
GTCATCTCCGTCTCTCAGAGCACGGTGTATATAAACAATAAGGCTAAGATCATATCCAGTGATATCAT
CAGTACTAATGGGATTGTTCATATCATAGACAAATTGCTATCTCCCAAAAATTTGCTTATCACTCCCA
AAGACAACTCTGGAAGAATTCTGCAAAATCTTACGACTTTGGCAACAAACAATGGCTACATCAAATTT
AGCAACTTAATACAGGACTCAGGTTTGCTGAGTGTCATCACCGATCCCATCCACACCCCAGTCACTCT
CTTCTGGCCCACCGACCAAGCCCTCCATGCCCTCCATGCCCTACCTGCTGAACAACAGGACTTCCTGT
TCAACCAAGACAACAAGGACAAGCTGAAGGAGTATTTGAAGTTTCATGTGATACGAGATGCCAAGGTT
TTAGCTGTGGATCTTCCCACATCCACTGCCTGGAAGACCCTGCAAGGTTCAGAGCTGAGTGTGAAATG
TGGAGCTGGCAGGGACATCGGTGACCTCTTTCTGAATGGCCAAACCTACAGAATTGTGCAGCGGGAGC
TCTTGTTTGACCTGGGTGTGGCCTACGGCATTGACTGTCTGCTGATTGATCCCACCCTGGGGGGCCGC
TGTGACACCTTTACTACTTTCGATGCCTCGGGGGAGTGTGGGAGCTGTGTCAATACTCCCAGCTGCCC
AAGGTGGAGTAAACCAAAGGGTGTGAAGCAGAAGTGTCTCTACAACCTGCCCTTCAAGAGGAACCTGG
AAGGCTGCCGGGAGCGGTGCAGCCTGGTGATACAGATCCCCAGGTGCTGCAAGGGCTACTTCGGGCGA
GACTGTCAGGCCTGCCCTGGAGGACCAGATGCCCCGTGTAATAACCGGGGTGTCTGCCTTGATCAGTA
CTCGGCCACCGGAGAGTGTAAATGCAACACCGGCTTCAATGGGACGGCGTGTGAGATGTGCTGGCCGG
GGAGATTTGGGCCTGATTGTCTGCCCTGTGGCTGCTCAGACCACGGACAGTGCGATGATGGCATCACG
GGCTCCGGGCAGTGCCTCTGTGAAACGGGGTGGACAGGCCCCTCGTGTGACACTCAGGCAGTTTTGCC
TGCAGTGTGTACGCCTCCTTGTTCTGCTCATGCCACCTGTAAGGAGAACAACACGTGTGAGTGTAACC
TGGATTATGAAGGTGACGGAATCACATGCACAGTTGTGGATTTCTGCAAACAGGACAACGGGGGCTGT
GCAAAGGTGGCCAGATGCTCCCAGAAGGGCACGAAGGTCTCCTGCAGCTGCCAGAAGGGATACAAAGG
GGACGGGCACAGCTGCACAGAGATAGACCCCTGTGCAGACGGCCTTAACGGAGGGTGTCACGAGCACG
CCACCTGTAAGATGACAGGCCCGGGCAAGCACAAGTGTGAGTGTAAAAGTCACTATGTCGGAGATGGG
CTGAACTGTGAGCCGGAGCAGCTGCCCATTGACCGCTGCTTACAGGACAATGGGCAGTGCCATGCAGA
CGCCAAATGTGTCGACCTCCACTTCCAGGATACCACTGTTGGGGTGTTCCATCTACGCTCCCCACTGG
GCCAGTATAAGCTGACCTTTGACAAAGCCAGAGAGGCCTGTGCCAACGAAGCTGCGACCATGGCAACC
TACAACCAGCTCTCCTATGCCCAGAAGGCCAAGTACCACCTGTGCTCAGCAGGCTGGCTGGAGACCGG
GCGGGTTGCCTACCCCACAGCCTTCGCCTCCCAGAACTGTGGCTCTGGTGTGGTTGGGATAGTGGACT
ATGGACCTAGACCCAACAAGAGTGAAATGTGGGATGTCTTCTGCTATCGGATGAAAGGAAGTGCTGGC
CTATTCCAACAGCTCAGCTCGAGGCCGTGCATTTCTAGAACACCTGACTGACCTGTCCATCCGCGGCA
CCCTCTTTGTGCCACAGAACAGTGGGCTGGGGGAGAATGAGACCTTGTCTGGGCGGGACATCGAGCAC
CACCTCGCCAATGTCAGCATGTTTTTCTACAATGACCTTGTCAATGGCACCACCCTGCAAACGAGGCT
GGGAAGCAAGCTGCTCATCACTGCCAGCCAGGACCCACTCCAACCGACGGAGACCAGGTTTGTTGATC
GAAGAGCCATTCTGCAGTGGGACATCTTTGCCTCCAATGGGATCATTCATGTCATTTCCAGGCCTTTA
AAAGCACCCCCTGCCCCCGTGACCTTGACCCACACTGGCTTGGGAGCAGGGATCTTCTTTGCCATCAT
CCTGGTGACTGGGGCTGTTGCCTTGGCTGCTTACTCCTACTTTCGGATAAACCGGAGAACAATCGGCT
TCCAGCATTTTGAGTCGGAAGAGGACATTAATGTTGCAGCTCTTGGCAAGCAGCAGCCTGAGAATATC
TCGAACCCCTTGTATGAGAGCACAACCTCAGCTCCCCCAGAACCTTCCTACGACCCCTTCACGGACTC
TGAAGAACGGCAGCTTGAGGGCAATGACCCCTTGAGGACACTGTGAGGGCCTGGACGGGAGATGCCAC
CCATCACTCACTGCCACCTGGGCCATCAACTGTGAATTCTCAGCACCAGTTGCCTTTTAGGAACGTAA
AGTCCTTTAAGCACTCAGAAGCCATACCTCATCTCTCTGGCTGATCTGGGGGTTGTTTCTGTGGGTGA
GAGATGTGTTGCTGTGCCCACCCAGTACAGCTTCCTCCTCTGACCCTTTGGCTCTTCTTCCTTTGTAC
TCTTCAGCTGGCACCTGCTCCATTCTGCCCTACATGATGGGTAACTGTGATCTTTCTTCCCTGTTAGA
TTGTAAGCCTCCGTCTTTGTATCCCAGCCCCTAGCCCAGTGCCTGACACAGGAACTGTGCACAATAAA
GGTTTATGGAACAGAAACAAAGTCAAAAAAAAAAAAAAAAAAAAAAAAAAAAAAAAAAAAAAC

| | | | |
|---|---|---|---|
| NOV9k, CG50736-10 | SEQ ID NO: 228 | 2417 aa | MW at 262971.8kD |
| Protein Sequence | | | |

MERQETGNSKTRYHATAIVQAKHDKGLNKNGTSGDEEQKIKVGDRDRENKGFDGLLDVWNTLNFIHPC
FAVCNCVHGVCNSGLDGDGTCECYSAYTGPKCDKLTENFHTSHLTLWPVHDSKHWGSLRHQNMNGTCS
SGGGKGDPDVYQNGLIFHGGGTSGGLSSSRNRRSSVKRPEKWKGDDRDGGGKEGQQRRRADTESSLQR
GHIKTPLPHRQGEARITETTGNCVSAGMTGTNANHTKVHPTVQSLTEYDSFQTHSTSRLKEFEKQQVK
ERFSDPPLMQAIKPSHEKYPPYAQRKGTSLSPKTQGHGDDEQALLSFLHSITLSLYLYPTTFFHDSPV
FIKPGIKTLRLNHFFGSSFPYEGSSVIXXMGIEVWKNWCQNADTLAAAPAPSLNVQPCSAQKIPDVRL
PLKMKTNWNANAFPITEAMANTATPSIHVYEKSATLMLIVRTWDQIGTVVHAKKATVGMAKCACLWTP
AKLTLETALQSLQCANNMGLDRCICQKGYVGDGLTCYGNIMERLRELNTEPRGKWQGRLTSFISLLES
IQIVSVQLSEFSQREPTCVNTKSIASNLEGPLVPLSNHYPLQVNELLVDNKAAQYFVKLHIIAGQNNI
EYMNNTDMFYTLTGKSGEIFNSDKDNQIKLKLHGGKKKVKIIQGDIIASNGLLHILDRAMDKLEPTFE
SNNEETNLGHALDEDGVGGPYTIFVPNNEALNNMKDGTLDYLLSPELEVATLISTPHIRSMANQLIQF
NTTDNGQILANDVAMEEIEITAKNGRIYTLTGVLIPPSIVPILPHRCDETKREMKLGTCVSCSLVYWS
RCPANSEPTALFTHRCVYSGRFGSLKSGCARYCNATVKCADSLGGNGTCICEEGFQGSQCQFCSDPNK
YGPRCNKKCLCVHGTCNNRIDSDGACLTGTCRDGSAGRLCDKQTSACGPYVQFCHIHATCEYSNGTAS
CICKAGYEGDGTLCSEMDPCTGLTPGGCSRNAECIKTGTGTHTCVCQQGWTGNGRDCSEINNCLLPSA
GGCHDNASCLYVGPGQNECECKKGFRGNGIDCEPITSCLEQTGKCHPLASCQSTSSGVWSCVCQEGYE
GDGFLCYGNAAVELSFLSEAAIFNRWINNASLQPTLSATSNLTVLVPSQQATEDMDQDEKSFWLSQSN
IPALIKYHMLLGTYRVADLQTLSSSDMLATSLQGNFLHLAKVDGNITIEGASIVDGDNAATNGVIHII
NKVLVPQRRLTGSLPNLLMRLEQMPDYSIFRGYIIQYNLANAIEAADAYTVFAPNNNAIENYIREKKV
LSLEEDVLRYHVVLEEKLLKNDLHNGMHRETMLGFSYFLSFFLHNDQLYVNEAPINYTNVATDKGVIH
GLGKVLEIQKNRCDNNDTTIIRGRCRTCSSELTCPFGTKSLGNEKRRCIYTSYFMGRRTLFIGCQPKC
VRTVITRECCAGFFGPQCQPCPGNAQNVCFGNGICLDGVNGTGVCECGEGFSGTACETCTEGKYGIHC
DQACSCVHGRCNQGPLGDGSCDCDVGWRGVHCDNATTEDNCNGTCHTSANCLTNSDGTASCKCAAGFQ
GNGTICTAINACEISNGGCSAKADCKRTTPGRRVCTCKAGYTGDGIVCLEINPCLENHGGCDKNAECT
QTGPNQAACNCLPAYTGDGKVCTLINVCLTKNGGCSEFAICNHTGQVERTCTCKPNYIGDGFTCRGSI
YQELPKNPKTSQYFFQLQEHFVKDLVGPGPFTVFAPLSAAFDEEARVKLWDKYGLMPQVLRYHVVACH
QLLLENLKLISNATSLQGEPIVISVSQSTVYINNKAKIISSDIISTNGIVHIIDKLLSPKNLLITPKD
NSGRILQNLTTLATNNGYIKFSNLIQDSGLLSVITDPIHTPVTLFWPTDQALHALHALPAEQQDFLFN
QDNKDKLKEYLKFHVIRDAKVLAVDLPTSTAWKTLQGSELSVKCGAGRDIGDLFLNGQTYRIVQRELL
FDLGVAYGIDCLLIDPTLGGRCDTFTTFDASGECGSCVNTPSCPRWSKPKGVKQKCLYNLPFKRNLEG
CRERCSLVIQIPRCCKGYFGRDCQACPGGPDAPCNNRGVCLDQYSATGECKCNTGFNGTACEMCWPGR
FGPDCLPCGCSDHGQCDDGITGSGQCLCETGWTGPSCDTQAVLPAVCTPPCSAHATCKENNTCECNLD
YEGDGITCTVVDFCKQDNGGCAKVARCSQKGTKVSCSCQKGYKGDGHSCTEIDPCADGLNGGCHEHAT
CKMTGPGKHKCECKSHYVGDGLNCEPEQLPIDRCLQDNGQCHADAKCVDLHFQDTTVGVFHLRSPLGQ
YKLTFDKAREACANEAATMATYNQLSYAQKAKYHLCSAGWLETGRVAYPTAFASQNCGSGVVGIVDYG
PRPNKSEMWDVFCYRMKGSAGLFQQLSSRPCISRTPD

| | | |
|---|---|---|
| NOV9l, CG50736-11 | SEQ ID NO: 229 | 1599 bp |
| DNA Sequence | ORF Start: at 1 | ORF Stop: end of sequence |

AGAATTCTGCAAAATCTTACGACTTTGGCAACAAACAATGGCTACATCAAATTTAGCAACTTAATACA
GGACTCAGGTTTGCTGAGTGTCATCACCGATCCCATCCACACCCCAGTCACTCTCTTCTGGCCCACCG
ACCAAGCCCTCCATGCCCTACCTGCTGAACAACAGGACTTCCTGTTCAACCAAGACAACAAGGACAAG
CTGAAGGAGTATTTGAAGTTTCATGTGATACGAGATGCCAAGGTTTTAGCTGTGGATCTTCCCACATC
CACTGCCTGGAAGACCCTGCAAGGTTCAGAGCTGAGTGTGAAATGTGGAGCTGGCAGGGACATCGGTG
ACCTCTTTCTGAATGGCCAAACCTGCAGAATTGTGCAGCGGGAGCTCTTGTTTGACCTGGGTGTGGCC
TACGGCATTGACTGTCTGCTGATTGATCCCACCCTGGGGGGCCGCTGTGACACCTTTACTACTTTCGA
TGCCTCGGGTGTGAAGCAGAAGTGTCTCTACAACCTGCCCTTCAAGAGGAACCTGGAAGGCTGCCGGG
AGCGGTGCAGCCTGGTGATACAGATCCCCAGGTGCTGCAAGGGCTACTTCGGGCGAGACTGTCAGGCC
TGCCCTGGAGGACCAGATGCCCCGTGTAATAACCGGGGTGTCTGCCTTGATCAGTACTCGGCCACCGG
AGAGTGTAAATGCAACACCGGCTTCAATGGGACGGCGTGTGAGATGTGCTGGCCGGGGAGATTCGGGC
CTGATTGTCTGCCCTGTGGCTGCTCAGACCACGGACAGTGCGATGATGGCATCACGGGCTCCGGGCAG
TGCCTCTGTGAAACGGGGTGGACAGGCCCCTCGTGTGACACTCAGGCAGTTTTGCCTGCAGTGTGTAC
GCCTCCTTGTTCTGCTCATGCCACCTGTAAGGAGAACAACACGTGTGAGTGTAACCTGGATTATGAAG
GTGACGGAATCACATGCACAGTTGTGGATTTCTGCAAACAGGACAACGGGGGCTGTGCAAAGGTGGCC
AGATGCTCCCAGAAGGGCACGAAGGTCTCCTGCAGCTGCCAGAAGGGATACAAAGGGGACGGGCACAG
CTGCACAGAGATAGACCCCTGTGCAGACGGCCTTAACGGAGGGTGTCACGAGCACGCCACCTGTAAGA
TGACAGGCCCGGGCAAGCACAAGTGTGAGTGTAAAAGTCACTATGTCGGAGATGGGCTGAACTGTGAG
CCGGAGCAGCTGCCCATTGACCGCTGCTTACAGGACAATGGGCAGTGCCATGCAGACGCCAAATGTGT
CGACCTCCACTTCCAGGATACCACTGTTGGGGTGTTCCATCTACGCTCCCCACTGGGCCAGTATAAGC
TGACCTTTGACAAAGCCAGAGAGGCCTGTGCCAACGAAGCTGCGACCATGGCAACCTACAACCAGCTC
TCCTATGCCCAGAAGGCCAAGTACCACCTGTGCTCAGCAGGCTGGCTGGAGACCGGGCGGGTTGCCTA
CCCCACAGCCTTCGCCTCCCAGAACTGTGGCTCTGGTGTGGTTGGGATAGTGGACTATGGACCTAGAC
CCAACAAGAGTGAAATGTGGGATGTCTTCTGCTAT

| | | | |
|---|---|---|---|
| NOV9l, CG50736-11 | SEQ ID NO: 230 | 533 aa | MW at 57827.8kD |
| Protein Sequence | | | |

RILQNLTTLATNNGYIKFSNLIQDSGLLSVITDPIHTPVTLFWPTDQALHALPAEQQDFLFNQDNKDK
LKEYLKFHVIRDAKVLAVDLPTSTAWKTLQGSELSVKCGAGRDIGDLFLNGQTCRIVQRELLFDLGVA
YGIDCLLIDPTLGGRCDTFTTFDASGVKQKCLYNLPFKRNLEGCRERCSLVIQIPRCCKGYFGRDCQA
CPGGPDAPCNNRGVCLDQYSATGECKCNTGFNGTACEMCWPGRFGPDCLPCGCSDHGQCDDGITGSGQ
CLCETGWTGPSCDTQAVLPAVCTPPCSAHATCKENNTCECNLDYEGDGITCTVVDFCKQDNGGCAKVA
RCSQKGTKVSCSCQKGYKGDGHSCTEIDPCADGLNGGCHEHATCKMTGPGKHKCECKSHYVGDGLNCE
PEQLPIDRCLQDNGQCHADAKCVDLHFQDTTVGVFHLRSPLGQYKLTFDKAREACANEAATMATYNQL
YAQKAKYHLCSAGWLETGRVAYPTAFASQNCGSGVVGIVDYGPRPNKSEMWDVFCY

| | | |
|---|---|---|
| NOV9m, CG50736-12 | JSEQ ID NO: 231 | 1611 bp |
| DNA Sequence | ORF Start: at 7 | ORF Stop: at 1606 |

GGATCCAGAATTCTGCAAAATCTTACGACTTTGGCAACAAACAATGGCTACATCAAATTTAGCAACTT
AATACAGGACTCAGGTTTGCTGAGTGTCATCACCGATCCCATCCACACCCCAGTCACTCTCTTCTGGC
CCACCGACCAAGCCCTCCATGCCCTACCTGCTGAACAACAGGACTTCCTGTTCAACCAAGACAACAAG
GACAAGCTGAAGGAGTATTTGAAGTTTCATGTGATACGAGATGCCAAGGTTTTAGCTGTGGATCTTCC
CACATCCACTGCCTGGAAGACCCTGCAAGGTTCAGAGCTGAGTGTGAAATGTGGAGCTGGCAGGGACA
TCGGTGACCTCTTTCTGAATGGCCAAACCTGCAGAATTGTGCAGCGGGAGCTCTTGTTTGACCTGGGT
GTGGCCTACGGCATTGACTGTCTGCTGATTGATCCCACCCTGGGGGGCCGCTGTGACACCTTTACTAC
TTTCGATGCCTCGGGTGTGAAGCAGAAGTGTCTCTACAACCTGCCCTTCAAGAGGAACCTGGAAGGCT
GCCGGGAGCGGTGCAGCCTGGTGATACAGATCCCCAGGTGCTGCAAGGGCTACTTCGGGCGAGACTGT
CAGGCCTGCCCTGGAGGACCAGATGCCCCGTGTAATAACCGGGGTGTCTGCCTTGATCAGTACTCGGC
CACCGGAGAGTGTAAATGCAACACCGGCTTCAATGGGACGGCGTGTGAGATGTGCTGGCCGGGGAGAT
TCGGGCCTGATTGTCTGCCCTGTGGCTGCTCAGACCACGGACAGTGCGATGATGGCATCACGGGCTCC
GGGCAGTGCCTCTGTGAAACGGGGTGGACAGGCCCCTCGTGTGACACTCAGGCAGTTTTGCCTGCAGT
GTGTACGCCTCCTTGTTCTGCTCATGCCACCTGTAAGGAGAACAACACGTGTGAGTGTAACCTGGATT
ATGAAGGTGACGGAATCACATGCACAGTTGTGGATTTCTGCAAACAGGACAACGGGGGCTGTGCAAAG
GTGGCCAGATGCTCCCAGAAGGGCACGAAGGTCTCCTGCAGCTGCCAGAAGGGATACAAAGGGGACGG
GCACAGCTGCACAGAGATAGACCCCTGTGCAGACGGCCTTAACGGAGGGTGTCACGAGCACGCCACCT
GTAAGATGACAGGCCCGGGCAAGCACAAGTGTGAGTGTAAAAGTCACTATGTCGGAGATGGGCTGAAC
TGTGAGCCGGAGCAGCTGCCCATTGACCGCTGCTTACAGGACAATGGGCAGTGCCATGCAGACGCCAA
ATGTGTCGACCTCCACTTCCAGGATACCACTGTTGGGGTGTTCCATCTACGCTCCCCACTGGGCCAGT
ATAAGCTGACCTTTGACAAAGCCAGAGAGGCCTGTGCCAACGAAGCTGCGACCATGGCAACCTACAAC
CAGCTCTCCTATGCCCAGAAGGCCAAGTACCACCTGTGCTCAGCAGGCTGGCTGGAGACCGGGCGGGT
TGCCTACCCCACAGCCTTCGCCTCCCAGAACTGTGGCTCTGGTGTGGTTGGGATAGTGGACTATGGAC
CTAGACCCAACAAGAGTGAAATGTGGGATGTCTTCTGCTATCGGCGG

| | | | |
|---|---|---|---|
| NOV9m, CG50736-12 | SEQ ID NO: 232 | 533 aa | MW at 57827.8kD |
| Protein Sequence | | | |

RILQNLTTLATNNGYIKFSNLIQDSGLLSVITDPIHTPVTLFWPTDQALHALPAEQQDFLFNQDNKDK
LKEYLKFHVIRDAKVLAVDLPTSTAWKTLQGSELSVKCGAGRDIGDLFLNGQTCRIVQRELLFDLGVA
YGIDCLLIDPTLGGRCDTFTTFDASGVKQKCLYNLPFKRNLEGCRERCSLVIQIPRCCKGYFGRDCQA
CPGGPDAPCNNRGVCLDQYSATGECKCNTGFNGTACEMCWPGRFGPDCLPCGCSDHGQCDDGITGSGQ
CLCETGWTGPSCDTQAVLPAVCTPPCSAHATCKENNTCECNLDYEGDGITCTVVDFCKQDNGGCAKVA
RCSQKGTKVSCSCQKGYKGDGHSCTEIDPCADGLNGGCHEHATCKMTGPGKHKCECKSHYVGDGLNCE
PEQLPIDRCLQDNGQCHADAKCVDLHFQDTTVGVFHLRSPLGQYKLTFDKAREACANEAATMATYNQL
SYAQKAKYHLCSAGWLETGRVAYPTAFASQNCGSGVVGIVDYGPRPNKSEMWDVFCY

| | | |
|---|---|---|
| NOV9n, CG50736-13 | SEQ ID NO: 233 | 1668 bp |
| DNA Sequence | ORF Start: at 7 | ORF Stop: at 1663 |

GGATCCAGAATTCTGCAAAATCTTACGACTTTGGCAACAAACAATGGCTACATCAAATTTAGCAACTT
AATACAGGACTCAGGTTTGCTGAGTGTCATCACCGATCCCATCCACACCCCAGTCACTCTCTTCTGGC
CCACCGACCAAGCCCTCCATGCCCTACCTGCTGAACAACAGGACTTCCTGTTCAACCAAGACAACAAG
GACAAGCTGAAGGAGTATTTGAAGTTTCATGTGATACGAGATGCCAAGGTTTTAGCTGTGGATCTTCC
CACATCCACTGCCTGGAAGACCCTGCAAGGTTCAGAGCTGAGTGTGAAATGTGGAGCTGGCAGGGACA
TCGGTGACCTCTTTCTGAATGGCCAAACCTGCAGAATTGTGCAGCGGGAGCTCTTGTTTGACCTGGGT
GTGGCCTACGGCATTGACTGTCTGCTGATTGATCCCACCCTGGGGGGCCGCTGTGACACCTTTACTAC
TTTCGATGCCTCGGGGGAGTGTGGGAGCTGTGTCAATACTCCCAGCTGCCCAAGGTGGAGTAAACCAA
AGGGTGTGAAGCAGAAGTGTCTCTACAACCTGCCCTTCAAGAGGAACCTGGAAGGCTGCCGGGAGCGG
TGCAGCCTGGTGATACAGATCCCCAGGTGCTGCAAGGGCTACTTCGGGCGAGACTGTCAGGCCTGCCC
TGGAGGACCAGATGCCCCGTGTAATAACCGGGGTGTCTGCCTTGATCAGTACTCGGCCACCGGAGAGT
GTAAATGCAACACCGGCTTCAATGGGACGGCGTGTGAGATGTGCTGGCCGGGGAGATTCGGGCCTGAT
TGTCTGCCCTGTGGCTGCTCAGACCACGGACAGTGCAATGATGGCATCACGGGCTCCGGGCAGTGCCT
CTGTGAAACGGGGTGGACAGGCCCCTCGTGTGACACTCAGGCAGTTTTGCCTGCAGTGTGTACGCCTC
CTTGTTCTGCTCATGCCACCTGTAAGGAGAACAACACGTGTGAGTGTAACCTGGATTATGAAGGTGAC
GGAATCACATGCACAGTTGTGGATTTCTGCAAACAGGACAACGGGGGCTGTGCAAAGGTGGCCAGATG
CTCCCAGAAGGGCACGAAGGTCTCCTGCAGCTGCCAGAAGGGATACAAAGGGGACGGGCACAGCTGCA
CAGAGATAGACCCTTGTGCAGACGGCCTTAACGGAGGGTGTCACGAGCACGCCACCTGTAAGATGACA
GGCCCGGGCAAGCACAAGTGTGAGTGTAAAAGTCACTATGTCGGAGATGGGCTGAACTGTGAGCCGGA
GCAGCTGCCCATTGACCGCTGCTTACAGGACAATGGGCAGTGCCATGCAGACGCCAAATGTGTCGACC
TCCACTTCCAGGATACCACTGTTGGGGTGTTCCATCTACGCTCCCCACTGGGCCAGTATAAGCTGACC
TTTGACAGAGCCAGAGAGGCCTGTGCCAACGAAGCTGCGACCATGGCAACCTACAACCAGCTCTCCTA
TGCCCAGAAGGCCAAGTACCACCTGTGCTCAGCAGGCTGGCTGGAGACCGGGCGGGTTGCCTACCCCA
CAGCCTTCGCCTCCCAGAACTGTGGCTCTGGTGTGGTTGGGATAGTGGACTATGGACCTAGACCCAAC
AAGAGTGAAATGTGGGATGTCTTCTGCTATCGGCCG

| | | | |
|---|---|---|---|
| NOV9n, CG50736-13 | SEQ ID NO: 234 | 552 aa | MW at 59873.1kD |
| Protein Sequence | | | |

RILQNLTTLATNNGYIKFSNLIQDSGLLSVITDPIHTPVTLFWPTDQALHALPAEQQDFLFNQDNKDK
LKEYLKFHVIRDAKVLAVDLPTSTAWKTLQGSELSVKCGAGRDIGDLFLNGQTCRIVQRELLFDLGVA
YGIDCLLIDPTLGGRCDTFTTFDASGECGSCVNTPSCPRWSKPKGVKQKCLYNLPFKRNLEGCRERCS
LVIQIPRCCKGYFGRDCQACPGGPDAPCNNRGVCLDQYSATGECKCNTGFNGTACEMCWPGRFGPDCL
PCGCSDHGQCNDGITGSGQCLCETGWTGPSCDTQAVLPAVCTPPCSAHATCKENNTCECNLDYEGDGI
TCTVVDFCKQDNGGCAKVARCSQKGTKVSCSCQKGYKGDGHSCTEIDPCADGLNGGCHEHATCKNTGP
GKHKCECKSHYVGDGLNCEPEQLPIDRCLQDNGQCHADAKCVDLHFQDTTVGVFHLRSPLGQYKLTFD
RAREACANEAATMATYNQLSYAQKAKYHLCSAGWLETGRVAYPTAFASQNCGSGVVGIVDYGPRPNKS
EMWDVFCY

| | | |
|---|---|---|
| NOV9o, CG50736-14 | SEQ ID NO: 235 | 1668 bp |
| DNA Sequence | ORF Start: at 7 | ORF Stop: at 1663 |

GGATCCAGAATTCTGCAAAATCTTACGACTTTGGCAACAAACAATGGCTACATCAAATTTAGCAACTT
AATACAGGACTCAGGTTTGCTGAGTGTCATCACCGATCCCATCCACACCCCAGTCACTCTCTTCTGGC
CCACCGACCAAGCCCTCCATGCCCTACCTGCTGAACAACAGGACTTCCTGTTCAACCAAGACAACAAG
GACAAGCTGAAGGAGTATTTGAAGTTTCATGTGATACGAGATGCCAAGGTTTTAGCTGTGGATCTTCC
CACATCCACTGCCTGGAAGACCCTGCAAGGTTCAGAGCTGAGTGTGAAATGTGGAGCTGGCAGGGACA
TCGGTGACCTCTTTCTGAATGGCCAAACCTGCAGAATTGTGCAGCGGGAGCTCTTGTTTGACCTGGGT
GTGGCCTACGGCATTGACTGTCTGCTGATTGATCCCACCCTGGGGGGCCGCTGTGACACCTTCACTAC
TTTCGATGCCTCGGGGGAGTGTGGGAGCTGTGTCAATACTCCCAGCTGCCCAAGGTGGAGTAAACCAA
AGGGTGTGAAGCAGAAGTGTCTCTACAACCTGCCCTTCAAGAGGAACCTGGAAGGCTGCCGGGAGCGG
TGCAGCCTGGTGATACAGATCCCCAGGTGCTGCAAGGGCTACTTCGGGCGAGACTGTCAGGCCTGCCC
TGGAGGACCAGATGCCCCGTGTAATAACCGGGGTGTCTGCCTTGATCTGTACTCGGCCACCGGAGAGT
GTAAATGCAACACCGGCTTCAATGGGACGGCGTGTGAGATGTGCTGGCCGGGGAGATTCGGGCCTGAT
TGTCTGCCCTGTGGCTGCTCAGACCACGGACAGTGCGATGATGGCATCACGGGCTCCGGGCAGTGCCT
CTGTGAAACGGGGTGGACAGGCCCCTCGTGTGACACTCAGGCAGTTTTGCCTGCAGTGTGTACGCCTC
CTTGTTCTGCTCATGCCACCTGTAAGGAGAACAACACGTGTGAGTGTAACCTGGATTATGAAGGTGAC
GGAATCACATGCACAGTTGTGGATTTCTGCAAACAGGACAACGGGGGCTGTGCAAAGGTGGCCAGATG
CTCCCAGAAGGGCACGAAGGTCTCCTGCAGCTGCCAGAAGGGATACAAAGGGGACGGGCACAGCTGCA
CAGAGATAGACCCCTGTGCAGACGGCCTTAACGGAGGGTGTCACGAGCACGCCACCTGTAAGATGACA
GGCCCGGGCAAGCACAAGTGTGAGTGTAAAAGTCACTATGTCGGAGATGGGCTGAACTGTGAGCCGGA
GCAGCTGCCCATTGACCGCTGCTTACAGGACAATGGGCAGTGCCATGCAGACGCCAAATGTGTCGACC
TCCACTTCCAGGATACCACTGTTGGGGTGTTCCATCTACGCTCCCCACTGGGCCAGTATAAGCTGACC
TTTGACAAAGCCAGAGAGGCCTGTGCCAACGAAGCTGCGACCATGGCAACCTACAACCAGCTCTCCTA
TGCCCAGAAGGCCAAGTACCACCTGTGCTCAGCAGGCTGGCTGGAGACCGGGCGGGTTGCCTACCCCA
CAGCCTTCGCCTCCCAGAACTGTGGCTCTGGTGTGGTTGGGATAGTGGACTATGGACCTAGACCCAAC
AAGAGTGAAATGTGGGATGTCTTCTGCTATCGGCCG

| | | | |
|---|---|---|---|
| NOV9o, CG50736-14 | SEQ ID NO: 236 | 552 aa | MW at 59831.1kD |
| Protein Sequence | | | |

RILQNLTTLATNNGYIKFSNLIQDSGLLSVITDPIHTPVTLFWPTDQALHALPAEQQDFLFNQDNKDK
LKEYLKFHVIRDAKVLAVDLPTSTAWKTLQGSELSVKCGAGRDIGDLFLNGQTCRIVQRELLFDLGVA
YGIDCLLIDPTLGGRCDTFTTFDASGECGSCVNTPSCPRWSKPKGVKQKCLYNLPFKRNLEGCRERCS
LVIQIPRCCKGYFGRDCQACPGGPDAPCNNRGVCLDLYSATGECKCNTGFNGTACEMCWPGRFGPDCL
PCGCSDHGQCDDGITGSGQCLCETGWTGPSCDTQAVLPAVCTPPCSAHATCKENNTCECNLDYEGDGI
TCTVVDFCKQDNGGCAKVARCSQKGTKVSCSCQKGYKGDGHSCTEIDPCADGLNGGCHEHATCKMTGP
GKHKCECKSHYVGDGLNCEPEQLPIDRCLQDNGQCHADAKCVDLHFQDTTVGVFHLRSPLGQYKLTFD
KAREACANEAATMATYNQLSYAQKAKYHLCSAGWLETGRVAYPTAFASQNCGSGVVGIVDYGPRPNKS
EMWDVFCY

| | | |
|---|---|---|
| NOV9p, CG50736-15 | SEQ ID NO: 237 | 1668 bp |
| DNA Sequence | ORF Start: at 7 | ORF Stop: at 1663 |

GGATCCAGAATTCTGCAAAATCTTACGACTTTGGCAACAAACAATGGCTACATCAAATTTAGCAACTT
AATACAGGACTCAGGTTTGCTGAGTGTCATCACCGATCCCATCCACACCCCAGTCACTCTCTTCTGGC
CCACCGACCAAGCCCTCCATGCCCTACCTGCTGAACAACAGGACTTCCTGTTCAACCAAGACAACAAG
GACAAGCTGAAGGAGTATTTGAAGTTTCATGTGATACGAGATGCCAAGGTTTTAGCTGTGGATCTTCC
CACATCCACTGCCTGGAAGACCCTGCAAGGTTCAGAGCTGAGTGTGAAATGTGGAGCTGGCAGGGACA
TCGGTGACCTCTTTCTGAATGGCCAAACCTGCAGAATTGTGCAGCGGGAGCTCTTGTTTGACCTGGGT
GTGGCCTACGGCATTGACTGTCTGCTGATTGATCCCACCCTGGGGGGCCGCTGTGACACCTTTACTAC
TTTCGATGCCTCGGGGGAGTGTGGGAGCTGTGTCAATACTCCCAGCTGCCCAAGGTGGAGTAAACCAA
AGGGTGTGAAGCAGAAGTGTCTCTACAACCTGCCCTTCAAGAGGAACCTGGAAGGCTGCCGGGAGCGG
TGCAGTCTGGTGATACAGATCCCCAGGTGCTGCAAGGGCTACTTCGGGCGAGACTGTCAGGCCTGCCC
TGGAGGACCAGATGCCCCGTGTAATAACCGGGGTGTCTGCCTTGATCAGTACTCGGCCACCGGAGAGT
GTAAATGCAACACCGGCTTCAATGGGACGGCGTGTGAGATGTGCTGGCCGGGGAGATTCGGGCCTGAT
TGTCTGCCCTGCGGCTGCTCAGACCACGGACAGTGCGATGATGGCATCACGGGCTCCGGGCAGTGCCT
CTGTGAAACGGGGTGGACAGGCCCCTCGTGTGACACTCAGGCAGTTTTGCCTGCAGTGTGTACGCCTC
CTTGTTCTGCTCATGCCACCTGTAAGGAGAACAACACGTGTGAGTGTAACCTGGATTATGAAGGTGAC
GGAATCACATGCACAGTTGTGGATTTCTGCAAACAGGACAACGGGGGCTGTGCAAAGGTGGCCAGATG
CTCCCAGAAGGGCACGAAGGTCTCCTGCAGCTGCCAGAAGGGATACAAAGGGGACGGGCACAGCTGCA
CAGAGATAGACCCCTGTGCAGACGGCCTTAACGGAGGGTGTCACGAGCACGCCACCTGTAAGATGACA
GGCCCGGGCAAGCACAAGTGTGAGTGTAAAAGTCACTATGTCGGAGATGGGCTGAACTGTGAGCCGGA
GCAGCTGCCCATTGACCGCTGCTTACAGGACAATGGGCAGTGCCATGCAGACGCCAAATGTGTCGACC
TCCACTTCCAGGATACCACTGTTGGGGTGTTCCATCTACGCTCCCCACTGGGCCAGTATAAGCTGACC
TTTGACAAAGCCAGAGAGGCCTGTGCCAACGAAGCTGCGACCATGGCAACCTACAACCAGCTCTCCTA
TGCCCAGAAGGCCAAGTACCACCTGTGCTCAGCAGGCTGGCTGGAGACCGGGCGGGTTGCCTACCCCA
CAGCCTTCGCCTCCCAGAACTGTGGCTCTGGTGTGGTTGGGATAGTGGACTATGGACCTAGACCCAAC
AAGAGTGAAATGTGGGATGTCTTCTGCTATCGGCCG

| | | | |
|---|---|---|---|
| NOV9p, CG50736-15 | SEQ ID NO: 238 | 552 aa | MW at 59846.1kD |
| Protein Sequence | | | |

RILQNLTTLATNNGYIKFSNLIQDSGLLSVITDPIHTPVTLFWPTDQALHALPAEQQDFLFNQDNKDK
LKEYLKFHVIRDAKVLAVDLPTSTAWKTLQGSELSVKCGAGRDIGDLFLNGQTCRIVQRELLFDLGVA
YGIDCLLIDPTLGGRCDTFTTFDASGECGSCVNTPSCPRWSKPKGVKQKCLYNLPFKRNLEGCRERCS
LVIQIPRCCKGYFGRDCQACPGGPDAPCNNRGVCLDQYSATGECKCNTGFNGTACEMCWPGRFGPDCL
PCGCSDHGQCDDGITGSGQCLCETGWTGPSCDTQAVLPAVCTPPCSAHATCKENNTCECNLDYEGDGI
TCTVVDFCKQDNGGCAKVARCSQKGTKVSCSCQKGYKGDGHSCTEIDPCADGLNGGCHEHATCKMTGP
GKHKCECKSHYVGDGLNCEPEQLPIDRCLQDNGQCHADAKCVDLHFQDTTVGVFHLRSPLGQYKLTFD
KAREACANEAATMATYNQLSYAQKAKYHLCSAGWLETGRVAYPTAFASQNCGSGVVGIVDYGPRPNKS
EMWDVFCY

| | | |
|---|---|---|
| NOV9q, CG50736-16 | SEQ ID NO: 239 | 1668 bp |
| DNA Sequence | ORF Start: at 7 | ORF Stop: at 1663 |

GGATCCAGAATTCTGCAAAATCTTACGACTTTGGCAACAAACAATGGCTACATCAAATTTAGCAACTT
AATACAGGACTCAGGTTTGCTGAGTGTCATCACCGATCCCATCCACACCCCAGTCACTCTCTTCTGGC
CCACCGACCAAGCCCTCCATGCCCTACCTGCTGAACAACAGGACTTCCTGTTCAACCAAGACAACAAG
GACAAGCTGAAGGAGTATTTGAAGTTTCATGTGATACGAGATGCCAAGGTTTTAGCTGTGGATCTTCC
CACATCCACTGCCTGGAAGACCCTGCAAGGTTCAGAGCTGAGTGTGAAATGTGGAGCTGGCAGGGACA
TCGGTGACCTCTTTCTGAATGGCCAAACCTGCAGAATTGTGCAGCGGGAGCTCTTGTTTGACCTGGGT
GTGGCCTACGGCATTGACTGTCTGCTGATTGATCCCACCCTGGGGGGCCGCTGTGACACCTTTACTAC
TTTCGATGCCTCGGGGGAGTGTGGGAGCTGTGTCAATACTCCCAGCTGCCCAAGGTGGAGTAAACCAA
AGGGTGTGAAGCAGAAGTGTCTCTACAACCTGCCCTTCAAGAGGAACCTGGAAGGCTGCCGGGAGCGG
TGCAGCCTGGTGATACAGATCCCCAGGTGCTGCAAGGGCTACTTCGGGCGAGACTGTCAGGCCTGCCC
TGGAGGACCAGATGCCCCGTGTAATAACCGGGGTGTCTGCCTTGATCAGTACTCGGCCACCGGAGAGT
GTAAATGCAACACCGGCTTCAATGGGACGGCGTGTGAGATGTGCTGGCCGGGGAGATTTGGGCCTGAT
TGTCTGCCCTGTGGCTGCTCAGACCACGGACAGTGCGATGATGGCATCACGGGCTCCGGGCAGTGCCT
CTGTGAAACGGGGTGGACAGGCCCCTCGTGTGACACTCAGGCAGTTTTGCCTGCAGTGTGTACGCCTC
CTTGTTCTGCTCATGCCACCTGTAAGGAGAACAACACGTGTGAGTGTTACCTGGATTATGAAGGTGAC
GGAATCACATGCACAGTTGTGGATTTCTGCAAACAGGACAACGGGGGCTGTGCAAAGGTGGCCAGATG
CTCCCAGAAGGGCACGAAGGTCTCCTGCAGCTGCCAGAAGGGATACAAAGGGGACGGGCACAGCTGCA
CAGAGATAGACCCCTGTGCAGACGGCCTTAACGGAGGGTGTCACGAGCACGCCACCCGTAAGATGACA
GGCCCGGGCAAGCACAAGTGTGAGTGTAAAAGTCACTATGTCGGAGATGGGCTGAACTGTGAGCCGGA
GCAGCTGCCCATTGACCGCTGCTTACAGGACAATGGGCAGTGCCATGCAGACGCCAAATGTGTCGACC
TCCACTTCCAGGATACCACTGTTGGGGTGTTCCATCTACGCTCCCCACTGGGCCAGTATAAGCTGACC
TTTGACAAAGCCAGAGAGGCCTGTGCCAACGAAGCTGCGACCATGGCAACCTACAACCAGCTCTCCTA
TGCCCAGAAGGCCAAGTACCACCTGTGCTCAGCAGGCTGGCTGGAGACCGGGCGGGTTGCCTACCCCA
CAGCCTTCGCCTCCCAGAACTGTGGCTCTGGTGTGGTTGGGATAGTGGACTATGGACCTAGACCCAAC
AAGAGTGAAATGTGGGATGTCTTCTGCTATCGGCCG

| | | | |
|---|---|---|---|
| NOV9q, CG50736-16 | SEQ ID NO: 240 | 552 aa | MW at 59948.2kD |
| Protein Sequence | | | |

RILQNLTTLATNNGYIKFSNLIQDSGLLSVITDPIHTPVTLFWPTDQALHALPAEQQDFLFNQDNKDK
LKEYLKFHVIRDAKVLAVDLPTSTAWKTLQGSELSVKCGAGRDIGDLFLNGQTCRIVQRELLFDLGVA
YGIDCLLIDPTLGGRCDTFTTFDASGECGSCVNTPSCPRWSKPKGVKQKCLYNLPFKRNLEGCRERCS
LVIQIPRCCKGYFGRDCQACPGGPDAPCNNRGVCLDQYSATGECKCNTGFNGTACEMCWPGRFGPDCL
PCGCSDHGQCDDGITGSGQCLCETGWTGPSCDTQAVLPAVCTPPCSAHATCKENNTCECYLDYEGDGI
TCTVVDFCKQDNGGCAKVARCSQKGTKVSCSCQKGYKGDGHSCTEIDPCADGLNGGCHEHATRKMTGP
GKHKCECKSHYVGDGLNCEPEQLPIDRCLQDNGQCHADAKCVDLHFQDTTVGVFHLRSPLGQYKLTFD
KAREACANEAATMATYNQLSYAQKAKYHLCSAGWLETGRVAYPTAFASQNCGSGVVGIVDYGPRPNKS
EMWDVFCY

| | | |
|---|---|---|
| NOV9r, CG50736-17 | SEQ ID NO: 241 | 1668 bp |
| DNA Sequence | ORF Start: at 7 | ORF Stop: at 1663 |

GGATCCAGAATTCTGCAAAATCTTACGACTTTGGCAACAAACAATGGCTACATCAAATTTAGCAACTT
AATACAGGACTCAGGTTTGCTGAGTGTCATCACCGATCCCATCCACACCCCAGTCACTCTCTTCTGGC
CCACCGACCAAGCCCTCCATGCCCTACCTGCTGA~CAACAGGACTTCCTGTTCAACCAAGACAACAAG
GACAAGCTGAAGGAGTATTTGAAGTTTCATGTGATACGAGATGCCAAGGTTTTAGCTGTGGATCTTCC
CACATCCACTGCCTGGAAGACCCTGCAAGGTTCAGAGCTGAGTGTGAAATGTGGAGCTGGCAGGGACA
TCGGTGACCTCTTTCTGAATGGCCAAACCTGCAGAATTGTGCAGCGGGAGCTCTTGTTTGACCTGGGT
GTGGCCTACGGCATTGACTGTCTGCTGATTGATCCCACCCTGGGGGGCCGCTGTGACACCTTTACTAC
TTTCGATGCCTCGGGGGAGTGTGGGAGCTGTGTCAATACTCCCAGCTGCCCAAGGTGGAGTAAACCAA
AGGGTGTGAAGCAGAAGTGTCTCTACAACCTGCCCTTCAAGAGGAACCTGGAAGGCTGCCGGGAGCGG
TGCAGCCTGGTGATACAGATCCCCAGGTGCTGCAAGGGCTACTTCGGGCGAGACTGTCAGGCCTGCCC
TGGAGGACCAGATGCCCCGTGTAATAACCGGGGTGTCTGCCTTGATCAGTACTCGGCCACCGGAGAGT
GTAAATGCAACACCGGCTTCAATGGGACGGCGTGTGAGATGTGCTGGCCGGGGAGATTCGGGCCTGAT
TGTCTGCCCTGTGGCTGCTCAGACCACGGACAGTGCGATGATGGCATCACGGGCTCCGGGCAGTGCCT
CTGTGAAACGGGGTGGACAGGCCCCTCGTGTGACACTCAGGCAGTTTTGCCTGCAGTGTGTACGCCTC
CTTGTTCTGCTCATGCCACCTGTAAGGAGAACAACACGTGTGAGTGTAACCTGGATTATGAAGGTGAC
GGAATCACATGCACAGTTGTGGATTTCTGCAAACAGGACAACGGGGGCTGTGCAAAGGTGGCCAGATG
CTCCCAGAAGGGCACGAAGGTCTCCTGCAACTGCCAGAAGGGGTACAAAGGGGACGGGCACAGCTGCA
CAGAGATAGACCCCTGTGCAGACGGCCTTAACGGAGGGTGTCACGAGCACGCCACCTGTAAGATGACA
GGCCCGGGCAAGCACAAGTGTGAGTGTAAAAGTCACTATGTCGGAGATGGGCTGAACTGTGAGCCGGA
GCAGCTGCCCATTGACCGCTGCTTACAGGACAATGGGCAGTGCCATGCAGACGCCAAATGTGTCGACC
TCCACTTCCAGGATACCACTGTTGGGGTGTTCCATCTACGCTCCCCACTGGGCCAGTATAAGCTGACC
TTTGACAAAGCCAGAGAGGCCTGTGCCAACGAAGCTGCGACCATGGCAACCTACAACCAGCTCTCCTA
TGCCCAGAAGGCCAAGTACCACCTGTGCTCAGCAGGCTGGCTGGAGACCGGGCGGGTTGCCTACCCCA
CAGCCTTCGCCTCCCAGAACTGTGGCTCTGGTGTGGTTGGGATAGTGGACTATGGACCTAGACCCAAC
AAGAGTGAAATGTGGGATGTCTTCTGCTATCGGCCG

| | | | |
|---|---|---|---|
| NOV9r, CG50736-17 | SEQ ID NO: 242 | 552 aa | MW at 59873.1kD |
| Protein Sequence | | | |

RILQNLTTLATNNGYIKFSNLIQDSGLLSVITDPIHTPVTLFWPTDQALHALPAEQQDFLFNQDNKDK
LKEYLKFHVIRDAKVLAVDLPTSTAWKTLQGSELSVKCGAGRDIGDLFLNGQTCRIVQRELLFDLGVA
YGIDCLLIDPTLGGRCDTFTTFDASGECGSCVNTPSCPRWSKPKGVKQKCLYNLPFKRNLEGCRERCS
LVIQIPRCCKGYFGRDCQACPGGPDAPCNNRGVCLDQYSATGECKCNTGFNGTACEMCWPGRFGPDCL
PCGCSDHGQCDDGITGSGQCLCETGWTGPSCDTQAVLPAVCTPPCSAHATCKENNTCECNLDYEGDGI
TCTVVDFCKQDNGGCAKVARCSQKGTKVSCNCQKGYKGDGHSCTETDPCADGLNGGCHEHATCKMTGP
GKHKCECKSHYVGDGLNCEPEQLPIDRCLQDNGQCHADAKCVDLHFQDTTVGVFHLRSPLGQYKLTFD
KAREACANEAATMATYNQLSYAQKAKYHLCSAGWLETGRVAYPTAFASQNCGSGVVGIVDYGPRPNKS
EMWDVFCY

| | | |
|---|---|---|
| NOV9s, CG50736-18 | SEQ ID NO: 243 | 1668 bp |
| DNA Sequence | ORF Start: at 7 | ORF Stop: at 1663 |

GGATCCAGAATTCTGCAAAATCTTACGACTTTGGCAACAAACAATGGCTACATCAAATTTAGCAACTT
AATACAGGACTCAGGTTTGCTGAGTGTCATCACCGATCCCATCCACACCCCAGTCACTCTCTTCTGGC
CCACCGACCAAGCCCTCCATGCCCTACCTGCTGAACAACAGGACTTCCTGTTCAACCAAGACAACAAG
GACAAGCTGAAGGAGTATTTGAAGTTTCATGTGATACGAGATGCCAAGGTTTTAGCTGTGGATCTTCC
CACATCCACTGCCTGGAAGACCCTGCAAGGTTCAGAGCTGAGTGTGAAATGTGGAGCTGGCAGGGACA
TCGGTGACCTCTTTCTGAATGGCCAAACCTGCAGAATTGTGCAGCGGGAGCTCTTGTTTGACCTGGGT
GTGGCCTACGGCATTGACTGTCTGCTGATTGATCCCACCCTGGGGGGCCGCTGTGACACCTTTACTAC
TTTCGATGCCTCGGGGGAGTGTGGGAGCTGTGTCAATACTCCCAGCTGCCCAAGGTGGAGTAAACCAA
AGGGTGTGAAGCAGAGGTGTCTCTACAACCTGCCCTTCAAGAGGAACCTGGAAGGCTGCCGGGAGCGG
TGCAGCCTGGTGATACAGATCCCCAGGTGCTGCAAGGGCTACTTCGGGCGAGACTGTCAGGCCTGCCC
TGGAGGACCAGATGCCCCGTGTAATAACCGGGGTGTCTGCCTTGATCAGTACTCGGCCACCGGAGAGT
GTAAATGCAACACCGGCTTCAATGGGACGGCGTGTGAGATGTGCTGGCCGGGGAGATTCGGGCCTGAT
TGTCTGCCCTGTGGCTGCTCAGACCACGGACAGTGCGATGATGGCATCACGGGCTCCGGGCAGTGCCT
CTGTGAAACGGGGTGGACAGGCCCCTCGTGTGACACTCAGGCAGTTTTGCCTGCAGTGTGTACGCCTC
CTTGTTCTGCTCATGCCACCTGTAAGGAGAACAACACGTGTGAGTGTAACCTGGATTATGAAGGTGAC
GGAATCACATGCACAGTTGTGGATTTCTGCAAACAGGACAACGGGGGCTGTGCAAAGGTGGCCAGATG
CAGAGATAGACCCCTGTGCAGACGGCCTTAACGGAGGGTGTCACGAGCACGCCACCTGTAAGATGACA
GGCCCGGGCAAGCACAAGTGTGAGTGTAAAAGTCACTATGTCGGAGATGGGCTGAACTGTGAGCCGGA
GCAGCTGCCCATTGACCGCTGCTTACAGGACAATGGGCAGTGCCATGCAGACGCCAAATGTGTCGACC
TCCACTTCCAGGATACCACTGTTGGGGTGTTCCATCTACGCTCCCCACTGGGCCAGTATAAGCTGACC
TTTGACAAAGCCAGAGAGGCCTGTGCCAACGAAGCTGCGACCATGGCAACCTACAACCAGCTCTCCTA
TGCCCAGAAGGCCAAGTACCACCTGTGCTCAGCAGGCTGGCTGGAGACCGGGCGGGTTGCCTACCCCA
CAGCCTTCGCCTCCCAGAACTGTGGCTCTAGTGTGGTTGGGATAGTGGACTATGGACCTAGACCCAAC
AAGAGTGAAATGTGGGATGTCTTCTGCTATCGGCCG

| | | | |
|---|---|---|---|
| NOV9s, CG50736-18 | SEQ ID NO: 244 | 552 aa | MW at 59904.1kD |
| Protein Sequence | | | |

RILQNLTTLATNNGYIKFSNLIQDSGLLSVITDPIHTPVTLFWPTDQALHALPAEQQDFLFNQDNKDK
LKEYLKFHVIRDAKVLAVDLPTSTAWKTLQGSELSVKCGAGRDIGDLFLNGQTCRIVQRELLFDLGVA
YGIDCLLIDPTLGGRCDTFTTFDASGECGSCVNTPSCPRWSKPKGVKQRCLYNLPFKRNLEGCRERCS
LVIQIPRCCKGYFGRDCQACPGGPDAPCNNRGVCLDQYSATGECKCNTGFNGTACEMCWPGRFGPDCL
PCGCSDHGQCDDGITGSGQCLCETGWTGPSCDTQAVLPAVCTPPCSAHATCKENNTCECNLDYEGDGI
TCTVVDFCKQDNGGCAKVARCSQKGTKVSCSCQKGYKGDGHSCTEIDPCADGLNGGCHEHATCKNTGP
GKHKCECKSHYVGDGLNCEPEQLPIDRCLQDNGQCHADAKCVDLHFQDTTVGVFHLRSPLGQYKLTFD
KAREACANEAATMATYNQLSYAQKAKYHLCSAGWLETGRVAYPTAFASQNCGSSVVGIVDYGPRPNKS
EMWDVFCY

| | | |
|---|---|---|
| NOV9t, CG50736-19 | SEQ ID NO: 245 | 1668 bp |
| DNA Sequence | ORF Start: at 7 | ORF Stop: at 1663 |

GGATCCAGAATTCTGCAAAATCTTACGACTTTGGCAACAAACAATGGCTACATCAAATTTAGCAACTT
AATACAGGACTCAGGTTTGCTGAGTGTCATCACCGATCCCATCCACACCCCAGTCACTCTCTTCTGGC
CCACCGACCAAGCCCTCCATGCCCTACCTGCTGAACAACAGGACTTCCTGTTCAACCAAGACAACAAG
GACAAGCTGAAGGAGTATTTGAAGTTTCATGTGATACGAGATGCCAAGGTTTTAGCTGTGGATCTTCC
CACATCCACTGCCTGGAAGACCCTGCAAGGTTCAGAGCTGAGTGTGAAATGTGGAGCTGGCAGGGACA
TCGGTGACCTCTTTCTGAATGGCCAAACCTGCAGAATTGTGCAGCGGGAGCTCTTGTTTGACCTGGGT
GTGGCCTGCGGCATTGACTGTCTGCTGATTGATCCCACCCTGGGGGGCCGCTGTGACACCTTTACTAC
TTTCGATGCCTCGGGGGAGTGTGGGAGCTGTGTCAATACTCCCAGCTGCCCAAGGTGGAGTAAACCAA
AGGGTGTGAAGCAGAAGTGTCTCTACAACCTGCCCTTCAAGAGGAACCTGGAAGGCTGCCGGGAGCGG
TGCAGCCTGGTGATACAGATCCCCAGGTGCTGCAAGGGCTACTTCGGGCGAGACTGTCAGGCCTGCCC
TGGAGGACCAGATGCCCCGTGTAATAACCGGGGTGTCTGCCTTGATCAGTACTCGGCCACCGGAGAGT
GTAAATGCAACACCGGCTTCAATGGGACGGCGTGTGAGATGTGCTGGCCGGGGAGATTTGGGCCTGAT
TGTCTGCCCTGTGGCTGCTCAGACCACGGACAGTGCGATGATGGCATCACGGGCTCCGGGCAGTGCCT
CTGTGAAACGGGGTGGACAGGCCCCTCGTGTGACACTCAGGCAGTTTTGCCTGCAGTGTGTACGCCTC
CTTGTTCTGCTCATGCCACCTGTAAGGAGAACAACACGTGTGAGTGTAACCTGGATTATGAAGGTGAC
GGAATCACATGCACAGTTGTGGATTTCTGCAAACAGGACAACGGGGGCTGTGCAAAGGTGGCCAGATG
CTCCCAGAAGGGCACGAAGGTCTCCTGCAGCTGCCAGAAGGGATACAAAGGGGACGGGCACAGCTGCA
CAGAGATAGACCCCTGTGCAGACGGCCTTAACGGAGGGTGTCACGAGCACGCCACCTGTAAGATGACA
GGCCCGGGCAAGCACAAGTGTGAGTGTAAAAGTCACTATGTCGGAGATGGGCTGAACTGTGAGCCGGA
GCAGCTGCCCATTGACCGCTGCTTACAGGACAATGGGCAGTGCCATGCAGACGCCAAATGTGTCGACC
TCCACTTCCAGGATACCACTGTTGGGGTGTTCCATCTACGCTCCCCACTGGGCCAGTATAAGCTGACC
TTTGACAAAGCCAGAGAGGCCTGTGCCAACGAAGCTGCGACCATGGCAACCTACAACCAGCTCTCCTA
TGCCCAGAAGGCCAAGTACCACCTGTGCTCAGCAGGCTGGCTGGAGACCGGGCGGGTTGCCTACCCCA
CAGCCTTCGCCTCCCAGAACTGCGGCTCTGGTGTGGTTGGGATAGTGGACTATGGACCTAGACCCAAC
AAGAGTGAAATGTGGGATGTCTTCTGCTATCGGCCG

| | | | |
|---|---|---|---|
| NOV9t, CG50736-19 | SEQ ID NO: 246 | 552 aa | MW at 59786.0kD |
| Protein Sequence | | | |

RILQNLTTLATNNGYIKFSNLIQDSGLLSVITDPIHTPVTLFWPTDQALHALPAEQQDFLFNQDNKDK
LKEYLKFHVIRDAKVLAVDLPTSTAWKTLQGSELSVKCGAGRDIGDLFLNGQTCRIVQRELLFDLGVA
CGIDCLLIDPTLGGRCDTFTTFDASGECGSCVNTPSCPRWSKPKGVKQKCLYNLPFKRNLEGCRERCS
LVIQIPRCCKGYFGRDCQACPGGPDAPCNNRGVCLDQYSATGECKCNTGFNGTACEMCWPGRFGPDCL
PCGCSDHGQCDDGITGSGQCLCETGWTGPSCDTQAVLPAVCTPPCSAHATCKENNTCECNLDYEGDGI
TCTVVDFCKQDNGGCAKVARCSQKGTKVSCSCQKGYKGDGHSCTEIDPCADGLNGGCHEHATCKMTGP
GKHKCECKSHYVGDGLNCEPEQLPIDRCLQDNGQCHADAKCVDLHFQDTTVGVFHLRSPLGQYKLTFD
KAREACANEAATMATYNQLSYAQKAKYHLCSAGWLETGRVAYPTAFASQNCGSGVVGIVDYGPRPNKS
EMWDVFCY

| | | |
|---|---|---|
| NOV9u, CG50736-20 | SEQ ID NO: 247 | 1668 bp |
| DNA Sequence | ORF Start: at 7 | ORF Stop: at 1663 |

GGATCCAGAATTCTGCAAAATCTTACGACTTTGGCAACAAACAATGGCTACATCAAATTTAGCAACTT
AATACAGGACTCAGGTTTGCTGAGTGTCGTCACCGATCCCATCCACACCCCAGTCACTCTCTTCTGGC
CCACCGACCAAGCCCTCCATGCCCTACCTGCTGAACAACAGGACTTCCTGTTCAACCAAGACAACAAG
GACAAGCTGAAGGAGTATTTGAAGTTTCATGTGATACGAGATGCCAAGGTTTTAGCTGTGGATCTTCC
CACATCCACTGCCTGGAAGACCCTGCAAGGTTCAGAGCTGAGTGTGAAATGTGGAGCTGGCAGGGACA
TCGGTGACCTCTTTCTGAATGGCCAAACCTGCAGAATTGTGCAGCGGGAGCTCTTGTTTGACCTGGGT
GTGGCCTACGGCATTGACTGTCTGCTGATTGATCCCACCCTGGGGGGCCGCTGTGACACCTTTACTAC
TTTCGATGCCTCGGGGGAGTGTGGGAGCTGTGTCAATACTCCCAGCTGCCCAAGGTGGAGTAAACCAA
AGGGTGTGAAGCAGAAGTGTCTCTACAACCTGCCCTTCAAGAGGAACCTGGAAGGCTGCCGGGAGCGG
TGCAGCCTGGTGATACAGATCCCCAGGTGCTGCAAGGGCTACTTCGGGCGAGACTGTCAGGCCTGCCC
TGGAGGACCAGATGCCCCGTGTAATAACCGGGGTGTCTGCCTTGATCAGTACTCGGCCACCGGAGAGT
GTAAATGCAACACCGGCTTCAATGGGACGGCGTGTGAGATGTGCTGGCCGGGGAGATTCGGGCCTGAT
CTGTGAAACGGGGTGGACAGGCCCCTCGTGTGACACTCAGGCAGTTTTGCCTGCAGTGTGTACGCCTC
CTTGTTCTGCTCATGCCACCTGTAAGGAGAACAACACGTGTGAGTGTAACCTGGATTATGAAGGTGAC
GGAATCACATGCACAGTTGTGGATTTCTGCAAACAGGACAACGGGGGCTGTGCAAAGGTGGCCAGATG
CTCCCAGAAGGGCACGAAGGTCTCCTGCAGCTGCCAGAAGGGATACAAAGGGGACGGGCACAGCTGCA
CAGAGATAGACCCCTGTGCAGACGGCCTTAACGGAGGGTGTCACGAGCACGCCACCTGTAAGATGACA
GGCCCGGGCAAGCACAAGTGTGAGTGTAAAAGTCACTATGTCGGAGATGGGCTGAACTGTGAGCCGGA
GCAGCTGCCCATTGACCGCTGCTTACAGGACAATGGGCAGTGCCATGCAGACGCCAAATGTGTCGACC
TCCACTTCCAGGATACCACTGTTGGGGTGTTCCATCTACGCTCCCCACTGGGCCAGTATAAGCTGACC
TTTGACAAAGCCAGAGAGGCCTGTGCCAACGAAGCTGCGACCACGGCAACCTACAACCAGCTCTCCTA
TGCCCAGAAGGCCAAGTACCACCTGTGCTCAGCAGGCTGGCTGGAGACCGGGCGGGTTGCCTACCCCA
CAGCCTTCGCCTCCCAGAACTGTGGCTCTGGTGTGGTTGGGATAGTGGACTATGGACCTAGACCCAAC
AAGAGTGAAATGTGGGATGTCTTCTGCTATCGGCCG

| | | | |
|---|---|---|---|
| NOV9u, CG50736-20 | SEQ ID NO: 248 | 552 aa | MW at 59802.0kD |
| Protein Sequence | | | |

RILQNLTTLATNNGYIKFSNLIQDSGLLSVVTDPIHTPVTLFWPTDQALHALPAEQQDFLFNQDNKDK
LKEYLKFHVIRDAKVLAVDLPTSTAWKTLQGSELSVKCGAGRDIGDLFLNGQTCRIVQRELLFDLGVA
YGIDCLLIDPTLGGRCDTFTTFDASGECGSCVNTPSCPRWSKPKGVKQKCLYNLPFKRNLEGCRERCS
LVIQIPRCCKGYFGRDCQACPGGPDAPCNNRGVCLDQYSATGECKCNTGFNGTACEMCWPGRFGPDCL
PCGCSDHGQCDDGITGSGQCLCETGWTGPSCDTQAVLPAVCTPPCSAHATCKENNTCECNLDYEGDGI
TCTVVDFCKQDNGGCAKVARCSQKGTKVSCSCQKGYKGDGHSCTEIDPCADGLNGGCHEHATCKMTGP
GKHKCECKSHYVGDGLNCEPEQLPIDRCLQDNGQCHADAKCVDLHFQDTTVGVFHLRSPLGQYKLTFD
KAREACANEAATTATYNQLSYAQKAKYHLCSAGWLETGRVAYPTAFASQNCGSGVVGIVDYGPRPNKS
EMWDVFCY

| | | |
|---|---|---|
| NOV9v, CG50736-21 | SEQ ID NO: 249 | 1668 bp |
| DNA Sequence | ORF Start: at 7 | ORF Stop: at 1663 |

GGATCCAGAATTCTGCAAAATCTTACGACTTTGGCAACAAACAATGGCTACATCAAATTTAGCAACTT
AATACAGGACTCAGGTTTGCTGAGTGTCATCACCGATCCCATCCACACCCCAGTCACTCTCTTCTGGC
CCACCGACCAAGCCCTCCATGCCCTACCTGCTGAACAACAGGACTTCCTGTTCAACCAAGACAACAAG
GACAAGCTGAAGGAGTATTTGAAGTTTCATGTGATACGAGATGCCAAGGTTTTAGCTGTGGATCTTCC
CACATCCACTGCCTGGAAGACCCTTCAAGGTTCAGAGCTGAGTGTGAAATGTGGAGCTGGCAGGGACA
TCGGTGACCTCTTTCTGAATGGCCAAACCTGCAGAATTGTGCAGCGGGAGCTCTTGTTTGACCTGGGT
GTGGCCTACGGCATTGACTGTCTGCTGATTGATCCCACCCTGGGGGGCCGCTGTGACACCTTTACTAC
TTTCGATGCCTCGGGGGAGTGTGGGAGCTGTGTCAATACTCCCAGCTGCCCAAGGTGGAGTAAACCAA
AGGGTGTGAAGCAGAAGTGTCTCTACAACCTGCCCTTCAAGAGGAACCTGGAAGGCTGCCGGGAGCGG
TGCAGCCTGGTGATACAGATCCCCAGGTGCTGCAAGGGCTACTTCGGGCGAGACTGTCAGGCCTGCCC
TGGAGGACCAGATGCCCCGTGTAATAACCGGGGTGTCTGCCTTGATCAGTACTCGGCCACCGGAGAGT
GTAAATGCAACACCGGCTTCAATGGGACGGCGTGTGAGATGTGCTGGCCGGGGAGATTCGGGCCTGAT
TGTCTGCCCTGTGGCTGCTCAGACCACGGACAGTGCGATGATGGCATCACGGGCTCCGGGCAGTGCCT
CTGTGAAACGGGGTGGACAGGCCCCTCGTGTGACACTCAGGCAGTTTTGCCTGCAGTGTGTACGCCTC
CTTGTTCTGCTCATGCCACCTGTAAGGAGAACAACACGTGTGAGTGTAACCTGGATTATGAAGGTGAC
GGAATCACATGCACAGTTGTGGATTTCTGCAAACAGGACAACGGGGGCTGTGCAAAGGTGGCCAGATG
CTCCCAGAAGGGCACGAAGGTCTCCTGCAGCTGCCAGAAGGGATACAAAGGGGACGGGCACAGCTGCA
CAGAGATAGACCCCTGTGCAGACGGCCTTAACGGAGGGTGTCACGAGCACGCCACCTGTAAGATGACA
GGCCCGGGCAAGCACAAGTGTGAGTGTAAAAGTCACTATGTCGGAGATGGGCTGAACTGTGAGCCGGA
GCAGCTGCCCATTGACCGCTGCTTACAGGACAATGGGCAGTGCCATGCAGACGCCAAATGTGTCGACC
TCCACTTCCAGGATACCACTGTTGGGGTGTTCCATCTACGCTCCCCACTGGGCCAGTATAAGCTGACC
TTTGACAAAGCCAGAGAGGCCTGTGCCAACGAAGCTGCGACCATGGCAACCTACAACCAGCTCTCCTA
TGCCCAGAAGGCCAAGTACCACCTGTGCTCAGCAGGCTGGCTGGAGACCGGGCGGGTTGCCTACCCCA
CAGCCTTCGCCTCCCAGAACTGTGGCTCTGGTGTGGTTGGGATAGTGGACTATGGACCTAGACCCAAC
AAGAGTGAAATGTGGGATGTCTTCTGCTATCGGCCG

| | | | |
|---|---|---|---|
| NOV9V, CG50736-21 | SEQ ID NO: 250 | 552 aa | MW at 59846.1kD |
| Protein Sequence | | | |

RILQNLTTLATNNGYIKFSNLIQDSGLLSVITDPIHTPVTLFWPTDQALHALPAEQQDFLFNQDNKDK
LKEYLKFHVIRDAKVLAVDLPTSTAWKTLQGSELSVKCGAGRDIGDLFLNGQTCRIVQRELLFDLGVA
YGIDCLLIDPTLGGRCDTFTTFDASGECGSCVNTPSCPRWSKPKGVKQKCLYNLPFKRNLEGCRERCS
LVIQIPRCCKGYFGRDCQACPGGPDAPCNNRGVCLDQYSATGECKCNTGFNGTACEMCWPGRFGPDCL
PCGCSDHGQCDDGITGSGQCLCETGWTGPSCDTQAVLPAVCTPPCSAHATCKENNTCECNLDYEGDGI
TCTVVDFCKQDNGGCAKVARCSQKGTKVSCSCQKGYKGDGHSCTEIDPCADGLNGGCHEHATCKMTGP
GKHKCECKSHYVGDGLNCEPEQLPIDRCLQDNGQCHADAKCVDLHFQDTTVGVFHLRSPLGQYKLTFD
KAREACANEAATMATYNQLSYAQKAKYHLCSAGWLETGRVAYPTAFASQNCGSGVVGIVDYGPRPNKS
EMWDVFCY

| | | |
|---|---|---|
| NOV9w, CG50736-22 | SEQ ID NO: 251 | 4527 bp |
| DNA Sequence | ORF Start: ATG at 4 | ORF Stop: TGA at 4510 |

GACATGGACCAGGATGAGAAAAGCTTCTGGTTGTCACAGAGCAATATTCCAGCCCTAATAAAGTACCA
TATGCTACTAGGCACATACAGAGTGGCAGATCTGCAGACCCTGTCTTCTTCTGACATGTTGGCAACAT
CTTTGCAGGGCAACTTCCTTCACTTGGCAAAGGTGGATGGGAATATCACAATTGAAGGGGCCTCCATT
GTCGATGGGGACAACGCAGCCACAAATGGAGTGATACACATCATCAACAAGGTGCTGGTCCCACAAAG
ACGTCTAACTGGCTCCTTACCAAACCTGCTCATGCGGCTGGAACAGATGCCTGACTATTCCATCTTCC
GGGGCTACATCATTCAATATAATCTGGCGAATGCAATTGAGGCTGCCGATGCCTACACAGTGTTTGCT
CCAAACAACAATGCCATCGAGAATTACATCAGGGAGAAGAAAGTCTTGTCTCTAGAGGAGGACGTCCT
CCGGTATCATGTGGTCCTGGAGGAGAAACTCCTGAAGAATGACCTGCACAATGGCATGCATCGTGAGA
CCATGCTGGGTTTCTCCTATTTCCTTAGCTTCTTTCTCCACAATGACCAGCTCTATGTAAATGAGGCT
CCAATAAACTACACCAATGTAGCCACTGATAAGGGAGTGATCCATGGCTTGGGAAAAGTTCTGGAAAT
TCAGAAGAACAGATGTGATAATAATGACACTACTATTATACGAGGAAGATGTAGGACATGCTCCTCAG
AGCTGACCTGCCCATTCGGAACTAAATCTCTAGGTAATGAGAAGAGGAGATGCATCTATACCTCCTAT
TTCATGGGAAGACGAACCCTGTTTATTGGGTGCCAGCCAAAATGTGTGAGAACCGTCATTACGAGAGA
ATGCTGTGCCGGCTTCTTTGGCCCCCAATGCCAGCCCTGCCCAGGGAATGCCCAGAATGTCTGCTTTG
GTAATGGCATCTGTTTGGATGGAGTGAATGGCACAGGTGTGTGTGAGTGTGGGGAGGGCTTCAGCGGC
ACAGCCTGCGAGACCTGCACCGAGGGCAAGTACGGCATCCACTGTGACCAAGCATGTTCTTGTGTCCA
TGGGAGATGCAACCAAGGACCCTTGGGAGATGGCTCCTGTGACTGTGATGTTGGCTGGCGAGGAGTGC
ATTGTGACAATGCAACCACAGAAGACAACTGCAATGGGACATGCCATACCAGCGCCAACTGCCTCACC
AACTCAGATGGTACAGCTTCATGCAAGTGTGCAGCAGGATTCCAAGGAAACGGGACCATCTGCACAGC
AATCAATGCCTGTGAGATCAGCAATGGAGGTTGCTCTGCCAAGGCTGACTGTAAGAGAACCACCCCAG
GAAGGCGAGTGTGCACGTGCAAAGCAGGCTACACGGGTGATGGCATTGTGTGCCTGGAAATCAACCCG
TGTTTGGAGAACCATGGTGGCTGTGACAAGAATGCGGAGTGCACACAGACAGGACCCAACCAGGCTGC
CTGTAACTGTTTGCCAGCATACACTGGAGATGGAAAGGTCTGCACACTCATCAATGTCTGCTTAACTA
AAAATGGCGGCTGTGGTGAATTTGCCATCTGCAACCACACTGGGCAAGTAGAAAGGACTTGTACTTGC
AAGCCAAACTACATTGGAGATGGATTTACCTGCCGCGGCAGCATTTATCAGGAGCTTCCCAAGAACCC
GAAAACTTCCCAGTATTTCTTCCAGTTGCAGGAGCATTTCGTGAAAGATCTGGTCGGCCCAGGCCCCT
TCACTGTTTTTGCACCTTTATCTGCAGCCTTTGATGAGGAAGCTCGGGTTAAAGACTGGGACAAATAC
GGTTTAATGCCCCAGGTTCTTCGGTACCATGTGGTCGCCTGCCACCAGCTGCTTCTGGAAAACCTGAA
ATTGATCTCAAATGCTACTTCCCTCCAAGGAGAGCCAATAGTCATCTCCGTCTCTCAGAGCACGGTGT
ATATAAACAATAAGGCTAAGATCATATCCAGTGATATCATCAGTACTAATGGGATTGTTCATATCATA
GACAAATTGCTATCTCCCAAAAATTTGCTTATCACTCCCAAAGACAACTCTGGAAGAATTCTGCAAAA
TCTTACGACTTTGGCAACAAACAATGGCTACATCAAATTTAGCAACTTAATACAGGACTCAGGTTTGC
TGAGTGTCATCACCGATCCCATCCACACCCCAGTCACTCTCTTCTGGCCCACCGACCAAGCCCTCCAT
GCCCTACCTGCTGAACAACAGGACTTCCTGTTCAACCAAAGACACAAGGACAAGCTGAAGGAGTATTT
GAAGTTTCATGTGATACGAGATGCCAAGGTTTTAGCTGTGGATCTTCCCACATCCACTGCCTGGAAGA
CCCTGCAAGGTTCAGAGCTGAGTGTGAAATGTGGAGCTGGCAGGGACATCGGTGACCTCTTTCTGAAT
GGCCAAACCTGCAGAATTGTGCAGCGGGAGCTCTTGTTTGACCTGGGTGTGGCCTACGGCATTGACTG
TCTGCTGATTGATCCCACCCTGGGGGGCCGCTGTGACACCTTTACTACTTTCGATGCCTCGGGGGAGT
GTGGGAGCTGTGTCAATACTCCCAGCTGCCCAAGGTGGAGTAAACCAAAGGGTGTGAAGCAGAAGTGT
CTCTACAACCTGCCCTTCAAGAGGAACCTGGAAGGCTGCCGGGAGCGGTGCAGCCTGGTGATACAGAT
CCCCAGGTGCTGCAAGGGCTACTTCGGGCGAGACTGTCAGCCTCGCAACTCTAAAAGTGTCTGCCTTG
ATCAGTACTCGGCCACCGGAGAGTGTAAATGCAACACCGGCTTCAATGGGACGGCGTGTGAGATGTGC
TGGCCGGGGAGATTTGGGCCTGATTGTCTGCCCTGTGGCTGCTCAGACCACGGACAGTGCGATGATGG
CATCACGGGCTCCGGGCAGTGCCTCTGTGAAACGGGGTGGACAGGCCCCTCGTGTGACACTCAGGCAG
TTTTGCCTGCAGTGTGTACGCCTCCTTGTTCTGCTCATGCCACCTGTAAGGAGAACAACACGTGTGAG
TGTAACCTGGATTATGAAGGTGACGGAATCACATGCACAGTTGTGGATTTCTGCAAACAGGACAACGG
GGGCTGTGCAAAGGTGGCCAGATGCTCCCAGAAGGGCACGAAGGTCTCCTGCAGCTGCCAGAAGGGAT
ACAAAGGGGACGGGCACAGCTGCACAGAGATAGACCCCTGTGCAGACGGCCTTAACGGAGGGTGTCAC
GAGCACGCCACCTGTAAGATGACAGGCCCGGGCAAGCACAAGTGTGAGTGTAAAAGTCACTATGTCGG
AGATGGGCTGAACTGTGAGCCGGAGCAGCTGCCCATTGACCGCTGCTTACAGGACAATGGGCAGTGCC
ATGCAGACGCCAAATGTGTCGACCTCCACTTCCAGGATACCACTGTTGGGGTGTTCCATCTACGCTCC
CCACTGGGCCAGTATAAGCTGACCTTTGACAAAGCCAGAGAGGCCTGTGCCAACGAAGCTGCGACCAT
GGCAACCTACAACCAGCTCTCCTATGCCCAGAAGGCCAAGTACCACCTGTGCTCAGCAGGCTGGCTGG
AGACCGGGCGGGTTGCCTACCCCACAGCCTTCGCCTCCCAGAACTGTGGCTCTGGTGTGGTTGGGATA
GTGGACTATGGACCTAGACCCAACAAGAGTGAAATGTGGGATGTCTTCTGCTATCGGATGAAAGATGT
GAACTGCACCTGCAAGGTGGGCTATGTGGGAGATGGCTTCTCATGCAGTGGGAACCTGCTGCAGGTCC
TGATGTCCTTCCCCTCACTCACAAACTTCCTGACGGAAGTGCTGGCCTATTCCAACAGCTCAGCTCGA
GGCCGTGCATTTCTAGAACACCTGACTGACCTGTCCATCCGCGGCACCCTCTTTGTGCCACAGAACAG
TGGGCTGGGGGAGAATGAGACCTTGTCTGGGCGGGACATCGAGCACCACCTCGCCAATGTCAGCATGT
TTTTCTACAATGACCTTGTCAATGGCACCACCCTGCAAACGAGGGTGGGAAGCAAGCTGCTCATCACT
GCCAGCCAGGACCCACTCCAACCGACGGAGACCAGGTTTGTTGATGGAAGAGCCATTCTGCAGTGGGA
CATCTTTGCCTCCAATGGGATCATTCATGTCATTTCCAGGCCTTTAAAAGCACCCCCTGCCCCCGTGA
CCTTGACCCACACTGGCTTGGGAGCAGGGATCTTCTTTGCCATCATCCTGGTGACTGGGGCTGTTGCC
TTGGCTGCTTACTCCTACTTTCGGATAAACCGGAGAACAATCGGCTTCCAGCATTTTGAGTCGGAAGA
GGACATTAATGTTGCAGCTCTTGGCAAGCAGCAGCCTGAGAATATCTCGAACCCCTTGTATGAGAGCA
CAACCTCAGCTCCCCCAGAACCTTCCTACGACCCCTTCACGGACTCTGAAGAACGGCAGCTTGAGGGC
AATGACCCCTTGAGGACACTGTGAGGGCCTGGACGGGAG

| | | | |
|---|---|---|---|
| NOV9w, CG50736-22 | SEQ ID NO: 252 | 1502 aa | MW at 163589.6kD |
| Protein Sequence | | | |

MDQDEKSFWLSQSNIPALIKYHMLLGTYRVADLQTLSSSDMLATSLQGNFLHLAKVDGNITIEGASIV
DGDNAATNGVIHIINKVLVPQRRLTGSLPNLLMRLEQMPDYSIFRGYIIQYNLANAIEAADAYTVFAP
NNNAIENYIREKKVLSLEEDVLRYHVVLEEKLLKNDLHNGMHRETMLGFSYFLSFFLHNDQLYVNEAP
INYTNVATDKGVIHGLGKVLEIQKNRCDNNDTTIIRGRCRTCSSELTCPFGTKSLGNEKRRCIYTSYF
MGRRTLFIGCQPKCVRTVITRECCAGFFGPQCQPCPGNAQNVCFGNGICLDGVNGTGVCECGEGFSGT
ACETCTEGKYGIHCDQACSCVHGRCNQGPLGDGSCDCDVGWRGVHCDNATTEDNCNGTCHTSANCLTN
SDGTASCKCAAGFQGNGTICTAINACEISNGGCSAKADCKRTTPGRRVCTCKAGYTGDGIVCLEINPC
LENHGGCDKNAECTQTGPNQAACNCLPAYTGDGKVCTLINVCLTKNGGCGEFAICNHTGQVERTCTCK
PNYIGDGFTCRGSIYQELPKNPKTSQYFFQLQEHFVKDLVGPGPFTVFAPLSAAFDEEARVKDWDKYG
LMPQVLRYHVVACHQLLLENLKLISNATSLQGEPIVISVSQSTVYINNKAKIISSDIISTNGIVHIID
KLLSPKNLLITPKDNSGRILQNLTTLATNNGYIKFSNLIQDSGLLSVITDPIHTPVTLFWPTDQALHA
LPAEQQDFLFNQDNKDKLKEYLKFHVIRDAXVLAVDLPTSTAWKTLQGSELSVKCGAGRDIGDLFLNG
QTCRIVQRELLFDLGVAYGIDCLLIDPTLGGRCDTFTTFDASGECGSCVNTPSCPRWSKPKGVKQKCL
YNLPFKRNLEGCRERCSLVIQIPRCCKGYFGRDCQPRNSKSVCLDQYSATGECKCNTGFNGTACEMCW
PGRFGPDCLPCGCSDHGQCDDGITGSGQCLCETGWTGPSCDTQAVLPAVCTPPCSAHATCKENNTCEC
NLDYEGDGITCTVVDFCKQDNGGCAKVARCSQKGTKVSCSCQKGYKGDGHSCTEIDPCADGLNGGCHE
HATCKMTGPGKHKCECKSHYVGDGLNCEPEQLPIDRCLQDNGQCHADAKCVDLHFQDTTVGVFHLRSP
LGQYKLTFDKAREACANEAATMATYNQLSYAQKAKYHLCSAGWLETGRVAYPTAFASQNCGSGVVGIV
DYGPRPNKSEMWDVFCYRMKDVNCTCKVGYVGDGFSCSGNLLQVLMSFPSLTNFLTEVLAYSNSSARG
RAFLEHLTDLSIRGTLFVPQNSGLGENETLSGRDIEHHLANVSMFFYNDLVNGTTLQTRVGSKLLITA
SQDPLQPTETRFVDGPAILQWDIFASNGIIHVISRPLKAPPAPVTLTHTGLGAGIFFAIILVTGAVAL
AAYSYFRINRRTIGFQHFESEEDINVAALGKQQPENISNPLYESTTSAPPEPSYDPFTDSEERQLEGN
DPLRTL

| | | |
|---|---|---|
| NOV9x, CG50736-23 | SEQ ID NO: 253 | 5416 bp |
| DNA Sequence | ORF Stan: ATG at 478 | ORF Stop: TGA at 5008 |

GTAGAATGCATCAAAACTGGCACGGGCACCCACACCTGCGTGTGTCAGCAGGGTTGGACAGGGAATGG
GAGAGACTGCTCGGAGATCAACAACTGCCTGCTGCCCAGTGCAGGCGGCTGCCACGACAACGCATCCT
GTTTGTATGTGGGTCCCGGGCAGAATGAGTGTGAGTGCAAGAAAGGATTTCGAGGAAATGGGATTGAC
TGTGCACCAATAACTTCATGCTTGGAACAAACCGGGAAATGTCATCCATTGGCAAGCTGTCAATCTAC
TTCGTCTGGTGTCTGGAGCTGTGTTTGTCAAGAGGGCTATGAAGGAGATGGCTTTCTGTGCTATGGAA
ACGCAGCAGTGGAATTGTCATTTCTCTCCGAAGCAGCTATATTTAACCGATGGATAAATAATGCTTCT
CTACAACCCACACTGTCAGCCACCTCAAACCTCACTGTCCTCGTGCCTTCCCAACAAGCTACTGAGGA
CATGGACCAGGATGAGAAAAGCTTCTGGTTGTCACAGAGCAATATTCCAGCCCTAATAAAGTACCATA
TGCTACTAGGCACATACAGAGTGGCAGATCTGCAGACCCTGTCTTCTTCTGACATGTTGGCAACATCT
TTGCAGGGCAACTTCCTTCACTTGGCAAAGGTGGATGGGAATATCACAATTGAAGGGGCCTCCATTGT
CGATGGGGACAACGCAGCCACAAATGGAGTGATACACATCATCAACAAGGTGCTGGTCCCACAAAGAC
GTCTAACTGGCTCCTTACCAAACCTGCTCATGCGGCTGGAACAGATGCCTGACTATTCCATCTTCCGG
GGCTACATCATTCAATATAATCTGGCGAATGCAATTGAGGCTGCCGATGCCTACACAGTGTTTGCTCC
AAACAACAATGCCATCGAGAATTACATCAGGGAGAAGAAAGTCTTGTCTCTAGAGGAGGACGTCCTCC
GGTATCATGTGGTCCTGGAGGAGAAACTCCTGAAGAATGACCTGCACAATGGCATGCATCGTGAGACC
ATGCTGGGTTTCTCCTATTTCCTTAGCTTCTTTCTCCACAATGACCAGCTCTATGTAAATGAGGCTCC
AATAAACTACACCAATGTAGCCACTGATAAGGGAGTGATCCATGGCTTGGGAAAAGTTCTGGAAATTC
AGAAGAACAGATGTGATAATAATGACACTACTATTATACGAGGAAGATGTAGGACATGCTCCTCAGAG
CTGACCTGCCCATTCGGAACTAAATCTCTAGGTAATGAGAAGAGGAGATGCATCTATACCTCCTATTT
CATGGGAAGACGAACCCTGTTTATTGGGTGCCAGCCAAAATGTGTGAGAACCGTCATTACGAGAGAAT
GCTGTGCCGGCTTCTTTGGCCCCCAATGCCAGCCCTGCCCAGGGAATGCCCAGAATGTCTGCTTTGGT
AATGGCATCTGTTTGGATGGAGTGAATGGCACAGGTGTGTGTGAGTGTGGGGAGGGCTTCAGCGGCAC
AGCCTGCGAGACCTGCACCGAGGGCAAGTACGGCATCCACTGTGACCAAGCATGTTCTTGTGTCCATG
GGAGATGCAACCAAGGACCCTTGGGAGATGGCTCCTGTGACTGTGATGTTGGCTGGCGAGGAGTGCAT
TGTGACAATGCAACCACAGAAGACAACTGCAATGGGACATGCCATACCAGCGCCAACTGCCTCACCAA
CTCAGATGGTACAGCTTCATGCAAGTGTGCAGCAGGATTCCAAGGAAACGGGACCATCTGCACAGCAA
TCAATGCCTGTGAGATCAGCAATGGAGGTTGCTCTGCCAAGGCTGACTGTAAGAGAACCACCCCAGGA
AGGCGAGTGTGCACGTGCAAAGCAGGCTACACGGGTGATGGCATTGTGTGCCTGGAAATCAACCCGTG
TTTGGAGAACCATGGTGGCTGTGACAAGAATGCGGAGTGCACACAGACAGGACCCAACCAGGCTGCCT
GTAACTGTTTGCCAGCATACACTGGAGATGGAAAGGTCTGCACACTCATCAATGTCTGCTTAACTAAA
AATGGCGGCTGTAGTGAATTTGCCATCTGCAACCACACTGGGCAAGTAGAAAGGACTTGTACTTGCAA
GCCAAACTACATTGGAGATGGATTTACCTGCCGCGGCAGCATTTATCAGGAGCTTCCCAAGAACCCGA
AAACTTCCCAGTATTTCTTCCAGTTGCAGGAGCACTTCGTGAAAGATCTGGTCGGCCCAGGCCCCTTC
ACTGTTTTTGCACCTTTATCTGCAGCCTTTGATGAGGAAGCTCGGGTTAAAGACTGGGACAAATACGG
TTTAATGCCCCAGGTTCTTCGGTACCATGTGGTCGCCTGCCACCAGCTGCTTCTGGAAAACCTGAAAT
TGATCTCAAATGCTACTTCCCTCCAAGGAGAGCCAATAGTCATCTCCGTCTCTCAGAGCACGGTGTAT
ATAAACAATAAGGCTAAGATCATATCCAGTGATATCATCAGTACTAATGGGATTGTTCATATCATAGA
CAAATTGCTATCTCCCAAAAATTTGCTTATCACTCCCAAAGACAACTCTGGAAGAATTCTGCAAAATC
TTACGACTTTGGCAACAAACAATGGCTACATCAAATTTAGCAACTTAATACAGGACTCAGGTTTGCTG
AGTGTCATCACCGATCCCATCCACACCCCAGTCACTCTCTTCTGGCCCACCGACCAAGCCCTCCATGC
CCTACCTGCTGAACAACAGGACTTCCTGTTCAACCAAGACAACAAGGACAAGCTGAAGGAGTATTTGA
AGTTTCATGTGATACGAGATGCCAAGGTTTTAGCTGTGGATCTTCCCACATCCACTGCCTGGAAGACC
CTGCAAGGTTCAGAGCTGAGTGTGAAATGTGGAGCTGGCAGGGACATCGGTGACCTCTTTCTGAATGG
CCAAACCTGCAGAATTGTGCAGCGGGAGCTCTTGTTTGACCTGGGTGTGGCCTACGGCATTGACTGTC
TGCTGATTGATCCCACCCTGGGGGGCCGCTGTGACACCTTTACTACTTTCGATGCCTCGGGGGAGTGT
GGGAGCTGTGTCAATACTCCCAGCTGCCCAAGGTGGAGTAAACCAAAGGGTGTGAAGCAGAAGTGTCT
CTACAACCTGCCCTTCAAGAGGAACCTGGAAGGCTGCCGGGAGCGGTGCAGCCTGGTGATACAGATCC
CCAGGTGCTGCAAGGGCTACTTCGGGCGAGACTGTCAGGCCTGCCCTGGAGGACCAGATGCCCCGTGT
AATAACCGGGGTGTCTGCCTTGATCAGTACTCGGCCACCGGAGAGTGTAAATGCAACACCGGCTTCAA
TGGGACGGCGTGTGAGATGTGCTGGCCGGGGAGATTTGGGCCTGATTGTCTGCCCTGTGGCTGCTCAG
ACCACGGACAGTGCGATGATGGCATCACGGGCTCCGGGCAGTGCCTCTGTGAAACGGGGTGGACAGGC
CCCTCGTGTGACACTCAGGCAGTTTTGCCTGCAGTGTGTACGCCTCCTTGTTCTGCTCATGCCACCTG
TAAGGAGAACAACACGTGTGAGTGTAACCTGGATTATGAAGGTGACGGAATCACATGCACAGTTGTGG
ATTTCTGCAAACAGGACAACGGGGGCTGTGCAAAGGTGGCCAGATGCTCCCAGAAGGGCACGAAGGTC
TCCTGCAGCTGCCAGAAGGGATACAAAGGGGACGGGCACAGCTGCACAGAGATAGACCCCTGTGCAGA
CGGCCTTAACGGAGGGTGTCACGAGCACGCCACCTGTAAGATGACAGGCCCGGGCAAGCACAAGTGTG
AGTGTAAAAGTCACTATGTCGGAGATGGGCTGAACTGTGAGCCGGAGCAGCTGCCCATTGACCGCTGC
TTACAGGACAATGGGCAGTGCCATGCAGACGCCAAATGTGTCGACCTCCACTTCCAGGATACCACTGT
TGGGGTGTTCCATCTACGCTCCCCACTGGGCCAGTATAAGCTGACCTTTGACAAAGCCAGAGAGGCCT
GTGCCAACGAAGCTGCGACCATGGCAACCTACAACCAGCTCTCCTATGCCCAGAAGGCCAAGTACCAC
CTGTGCTCAGCAGGCTGGCTGGAGACCGGGCGGGTTGCCTACCCCACAGCCTTCGCCTCCCAGAACTG
TGGCTCTGGTGTGGTTGGGATAGTGGACTATGGACCTAGACCCAACAAGAGTGAAATGTGGGATGTCT
TCTGCTATCGGATGAAAGATGTGAACTGCACCTGCAAGGTGGGCTATGTGGGAGATGGCTTCTCATGC
AGTGGGAACCTGCTGCAGGTCCTGATGTCCTTCCCCTCACTCACAAACTTCCTGACGGAAGTGCTGGC
CTATTCCAACAGCTCAGCTCGAGGCCGTGCATTTCTAGAACACCTGACTGACCTGTCCATCCGCGGCA
CCCTCTTTGTGCCACAGAACAGTGGGCTGGGGGAGAATGAGACCTTGTCTGGGCGGGACATCGAGCAC
CACCTCGCCAATGTCAGCATGTTTTTCTACAATGACCTTGTCAATGGCACCACCCTGCAAACGAGGCT
GGGAAGCAAGCTGCTCATCACTGCCAGCCAGGACCCACTCCAACCGACGGAGACCAGGTTTGTTGATG
GAAGAGCCATTCTGCAGTGGGACATCTTTGCCTCCAATGGGATCATTCATGTCATTTCCAGGCCTTTA
AAAGCACCCCCTGCCCCCGTGACCTTGACCCACACTGGCTTGGGAGCAGGGATCTTCTTTGCCATCAT
CCTGGTGACTGGGGCTGTTGCCTTGGCTGCTTACTCCTACTTTCGGATAAACCGGAGAACAATCGGCT
TCCAGCATTTTGAGTCGGAAGAGGACATTAATGTTGCAGCTCTTGGCAAGCAGCAGCCTGAGAATATC
TCGAACCCCTTGTATGAGAGCACAACCTCAGCTCCCCCAGAACCTTCCTACGACCCCTTCACGGACTC
TGAAGAACGGCAGCTTGAGGGCAATGACCCCTTGAGGACACTGTGAGGGCCTGGACGGGAGATGCCAC
CCATCACTCACTGCCACCTGGGCCATCAACTGTGAATTCTCAGCACCAGTTGCCTTTTAGGAACGTAP
AGTCCTTTAAGCACTCAGAAGCCATACCTCATCTCTCTGGCTGATCTGGGGGTTGTTTCTGTGGGTGA
GAGATGTGTTGCTGTGCCCACCCAGTACAGCTTCCTCCTCTGACCCTTTGGCTCTTCTTCCTTTGTAC
TCTTCAGCTGGCACCTGCTCCATTCTGCCCTACATGATGGGTAACTGTGATCTTTCTTCCCTGTTTGT
TAGATTGTAAGCCTCCGTCTTTGTATCCCAGCCCCTAGCCCAGTGCCTGACACAGGAACTGTGCACAA
TAAAGGTTTATGGAACAGAAACAAAGTCAACAGAAAAAAAAAAA

| | | | |
|---|---|---|---|
| NOV9x, CG50736-23 | SEQ ID NO: 254 | 1510 aa | MW at 164274.3kD |
| Protein Sequence | | | |

MDQDEKSFWLSQSNIPALIKYHMLLGTYRVADLQTLSSSDMLATSLQGNFLHLAKVDGNITIEGASIV
DGDNAATNGVIHIINKVLVPQRRLTGSLPNLLMRLEQMPDYSIFRGYIIQYNLANAIEAADAYTVFAP
NNNAIENYIREKKVLSLEEDVLRYHVVLEEKLLKNDLHNGMHRETMLGFSYFLSFFLHNDQLYVNEAP
INYTNVATDKGVIHGLGKVLEIQKNRCDNNDTTIIRGRCRTCSSELTCPFGTKSLGNEKRRCIYTSYF
MGRRTLFIGCQPKCVRTVITRECCAGFFGPQCQPCPGNAQNVCFGNGICLDGVNGTGVCECGEGFSGT
ACETCTEGKYGIHCDQACSCVHGRCNQGPLGDGSCDCDVGWRGVHCDNATTEDNCNGTCHTSANCLTN
SDGTASCKCAAGFQGNGTICTAINACEISNGGCSAKADCKRTTPGRRVCTCKAGYTGDGIVCLEINPC
LENHGGCDKNAECTQTGPNQAACNCLPAYTGDGKVCTLINVCLTKNGGCSEFAICNHTGQVERTCTCK
PNYIGDGFTCRGSIYQELPKNPKTSQYFFQLQEHFVKDLVGPGPFTVFAPLSAAFDEEARVKDWDKYG
LMPQVLRYHVVACHQLLLENLKLISNATSLQGEPIVISVSQSTVYINNKAKIISSDIISTNGIVHIID
KLLSPKNLLITPKDNSGRILQNLTTLATNNGYIKFSNLIQDSGLLSVITDPIHTPVTLFWPTDQALHA
LPAEQQDFLFNQDNKDKLKEYLKFHVIRDAKVLAVDLPTSTAWKTLQGSELSVKCGAGRDIGDLFLNG
QTCRIVQRELLFDLGVAYGIDCLLIDPTLGGRCDTFTTFDASGECGSCVNTPSCPRWSKPKGVKQKCL
YNLPFKRNLEGCRERCSLVIQIPRCCKGYFGRDCQACPGGPDAPCNNRGVCLDQYSATGECKCNTGFN
GTACEMCWPGRFGPDCLPCGCSDHGQCDDGITGSGQCLCETGWTGPSCDTQAVLPAVCTPPCSAHATC
KENNTCECNLDYEGDGITCTVVDFCKQDNGGCAKVARCSQKGTKVSCSCQKGYKGDGHSCTEIDPCAD
GLNGGCHEHATCKMTGPGKHKCECKSHYVGDGLNCEPEQLPIDRCLQDNGQCHADAKCVDLHFQDTTV
GVFHLRSPLGQYKLTFDKAREACANEAATMATYNQLSYAQKAKYHLCSAGWLETGRVAYPTAFASQNC
GSGVVGIVDYGPRPNKSEMWDVFCYRMKDVNCTCKVGYVGDGFSCSGNLLQVLMSFPSLTNFLTEVLA
YSNSSARGRAFLEHLTDLSIRGTLFVPQNSGLGENETLSGRDIEHHLANVSMFFYNDLVNGTTLQTRL
GSKLLITASQDPLQPTETRFVDGRAILQWDIFASNGIIHVISRPLKAPPAPVTLTHTGLGAGIFFAII
LVTGAVALAAYSYFRINPRTIGFQHFESEEDINVAALGKQQPENISNPLYESTTSAPPEPSYDPFTDS
EERQLEGNDPLRTL

| | | |
|---|---|---|
| NOV9y, CG50736-24 | SEQ ID NO: 255 | 7670 bp |
| DNA Sequence | ORF Start: ATG at 8 | ORF Stop: TGA at 7661 |

TTTCCTCATGATGCTACAACATTTAGTAATTTTTTGTCTTGGATTGGTTGTACAAAATTTCTGCTCCC
CAGCTGAAACCACAGGGCAGGCAAGAAGATGTGATAGGAAGTCTCTTCTTACAATTAGGACCGAGTGC
CGATCCTGCGCTCTCAACCTTGGAGTCAAGTGCCCGGATGGTTACACCATGATTACCAGTGGCTCTGT
AGGGGTTCGAGATTGCAGGTACACCTTTGAGGTCAGAACATACTCTCTGTCTCTCCCCGGATGCCGCC
ATATTTGTAGGAAGGACTATCTCCAACCTCGGTGTTGTCCTGGCCGCTGGGGCCCAGACTGTATAGAG
TGCCCAGGTGGAGCGGGGTCACCCTGCAATGGCAGAGGCAGTTGTGCTGAAGGCATGGAAGGAAATGG
AACCTGCTCCTGCCAAGAAGGGTTTGGTGGAACAGCCTGTGAAACCTGTGCTGACGACAACTTATTTG
GACCCAGCTGTTCATCAGTGTGCAACTGTGTGCATGGGGTGTGCAACAGTGGACTAGATGGCGATGGA
ACCTGTGAGTGCTACTCTGCGTACACTGGCCCCAAGTGTGACAAGCCCATCCCTGAATGTGCAGCCTT
GCTCTGCCCAGAAAATTCCAGATGTTCGCCTTCCACTGAAGATGAAAACAAACTGGAATGCAAATGCC
TTCCCAATTACCGAGGCGATGGCAAATACTGCGACCCCATCAATCCATGTTTACGAAAAATCTGCCAC
CCTCATGCTCATTGTACGTACCTGGGACCAAATCGGCACAGTTGTACATGCCAAGAAGGCTACCGTGG
GGATGGCCAAGTGTGCTTGCCTGTGGACCCCTGCCAAATTAACTTTGGAAACTGCCCTACAAAGTCTA
CAGTGTGCAAATATGATGGGCCTGGACAGTCTCACTGCGAGTGTAAGGAGCATTACCAGAATTTCGTA
CCTGGAGTGGGGTGCAGTATGACTGATATATGTAAATCAGATAACCCGTGTCATAGGAATGCAAATTG
CACCACCGTCGCACCAGGCCGAACTGAATGCATTTGCCAGAAAGGTTACGTGGGTGATGGCTTAACGT
GTTATGGAAACATTATGGAGCGACTCAGAGAATTAAATACTGAACCCAGAGGAAAATGGCAAGGAAGG
CTGACCTCTTTCATCTCACTCCTAGACAAAGCTTATGCCTGGCCACTGAGTAAGCTGGGACCCTTCAC
GGTGCTGTTACCTACAGACAAGGGACTGAAAGGATTCAATGTAAATGAGCTTTTGGTGGATAATAAAG
CTGCTCAATACTTTGTGAAACTCCACATAATTGCTGGTCAGATGAACATCGAATATATGAATAACACA
GACATGTTCTACACCTTGACTGGAAAGTCGGGGGAAATCTTCAACAGCGATAAGGACAATCAAATAAA
GCTTAAACTCCATGGAGGCAAAAAGAAGGTAAAAATTATACAAGGGGACATCATTGCTTCCAATGGGC
TTCTGCACATCCTTGACAGAGCCATGGACAAGTTAGAACACACATTTGAGAGCAACAATGAGCAAACC
ATAATGACAATGCTACAACCAAGGTACAGCAAGTTCAGATCTTTGTTAGAGGAAACCAATTTGGGACA
TGCCTTAGATGAGGATGGAGTTGGTGGACCATACACCATTTTTGTTCCAAATAATGAAGCATTGAATA
ACATGAAGGACGGCACTCTCGATTACCTCCTTTCTCCAGAGGGATCTCGGAAGCTTCTGGAACTCGTC
AGATACCACATTGTCCCATTTACCCAGCTTGAAGTGGCCACTCTCATCTCCACCCCTCACATCAGGAG
CATGGCCAACCAGCTCATACAGTTCAACACCACCGACAATGGACAGATTCTGGCAAATGATGTGGCAA
TGGAAGAAATTGAGATCACTGCCAAAAATGGCCGAATTTACACACTGACAGGAGTTCTCATTCCTCCC
TCCATTGTCCCGATTCTGCCCCATCGATGTGATGAAACAAAGAGAGAGATGAAACTGGGCACTTGTGT
GAGCTGTTCTCTGGTGTACTGGAGCAGATGTCCTGCTAACTCTGAGCCCACAGCACTCTTCACACACA
GATGTGTCTACAGTGGCAGGTTTGGGAGCCTGAAGAGCGGCTGTGCCCGGTACTGCAATGCCACTGTG
AAGATTCCAAAGTGCTGCAAAGGCTTCTATGGACCTGACTGCAACCAGTGTCCAGGAGGCTTCTCAAA
TCCATGCTCAGGAAATGGACAGTGTGCAGATAGCCTCGGCGGCAACGGGACATGCATTTGTGAGGAGG
GCTTCCAAGGCTCCCAGTGTCAGTTCTGCTCTGATCCCAATAAATACGGACCTCGGTGTAACAAAAAA
TGCCTGTGCGTTCACGGAACATGCAATAACAGGATAGACAGCGATGGGGCCTGCCTCACTGGCACATG
CAGAGACGGCTCTGCCGGGAGACTCTGTGATAAGCAGACCTCAGCCTGTGGGCCCTATGTGCAGTTCT
GTCACATCCACGCCACCTGTGAATACAGCAATGGGACAGCCAGTTGTATTTGCAAAGCAGGATATGAA
GGAGATGGAACTCTGTGTTCTGAGATGGACCCTTGCACAGGACTAACTCCAGGAGGCTGTAGCCGCAA
TGCAGAATGCATCAAAACTGGCACGGGCACCCACACCTGCGTGTGTCAGCAGGGTTGGACAGGGAATG
GGAGAGACTGCTCGGAGATCAACAACTGCCTGCTGCCCAGTGCAGGCGGCTGCCACGACAACGCATCC
TGTTTGTATGTGGGTCCCGGGCAGAATGAGTGTGAGTGCAAGAAAGGATTTCGAGGAAATGGGATTGA
CTGTGAACCAATAACTTCATGCTTGGAACAAACCGGGAAATGTCATCCATTGGCAAGCTGTCAATCTA
CTTCGTCTGGTGTCTGGAGCTGTGTTTGTCAAGAGGGCTATGAAGGAGATGGCTTTCTGTGCTATGGA
AACGCAGCAGTGGAATTGTCATTTCTCTCCGAAGCAGCTATATTTAACCGATGGATAAATAATGCTTC
TCTACAACCCACACTGTCAGCCACCTCAAACCTCACTGTCCTCGTGCCTTCCCAACAAGCTACTGAGG
ACATGGACCAGGATGAGAAAAGCTTCTGGTTGTCACAGAGCAATATTCCAGCCCTAATAAAGTACCAT
ATGCTACTAGGCACATACAGAGTGGCAGATCTGCAGACCCTGTCTTCTTCTGACATGTTGGCAACATC
TTTGCAGGGCAACTTCCTTCACTTGGCAAAGGTGGATGGGAATATCACAATTGAAGGGGCCTCCATTG
TCGATGGGGACAATGCAGCCACAAATGGAGTGATACACATCATCAACAAGGTGCTGGTCCCACAAAGA
CGTCTAACTGGCTCCTTACCAAACCTGCTCATGCGGCTGGAACAGATGCCTGACTATCCCATCTTCCG
GGGCTACATCATTCAATATAATCTGGCGAATGCAATTGAGGCTGCCGATGCCTACACAGTGTTTGCTC
CAAACAACAATGCCATCGAGAATTACATCAGGGAGAAGAAAGTCTTGTCTCTAGAGGAGGACGTCCTC
CGGTATCATGTGGTCCTGGAGGAGAAACTCCTGAAGAATGACCTGCACAATGGCATGCATCGTGAGAC
CATGCTGGGTTTCTCCTATTTCCTTAGCTTCTTTCTCCATAATGACCAGCTCTATGTAAATGAGGCTC
CAATAAACTACACCAATGTAGCCACTGATAAGGGAGTGATCCATGGCTTGGGAAAAGTTCTGGAAATT
CAGAAGAACAGATGTGATAATAATGCCACTACTATTATACGAGGAAGATGTAGGACATGCTCCTCAGA
GCTGACCTGCCCATTCGGAACTAAATCTCTAGGTAATGAGAAGAGGAGATGCATCTATACCTCCTATT
TCATGGGAAGACGAACCCTGTTTATTGGGTGCCAGCCAAAATGTGTGAGAACCGTCATTACGAGAGAA
TGCTGTGCCGGCTTCTTTGGCCCCCAATGCCAGCCCTGTCCAGGGAATGCCCAGAATGTCTGCTTTGG
TAATGGCATCTGTTTGGATGGAGTGAATGGCACAGGTGTGTGTGAGTGTGGGGAGGGCTTCAGCGGCA
CAGCCTGCGAGACCTGCACCGAGGGCAAGTACGGCATCCACTGTGACCAAGCATGTTCTTGTGTCCAT
GGGAGATGCAACCAAGGACCCTTGGGAGATGGCTCCTGTGACTGTGATGTTGGCTGGCGAGGAGTGCA
TTGTGACAATGCAACCACAGAAGACAACTGCAATGGGACATGCCATACCAGCGCCAACTGCCTCACCA
ACTCAGATGGTACAGCTTCATGCAAGTGTGCAGCAGGATTCCAAGGAAACGGGACCATCTGCACAGCA
ATCAATGCCTGTGAGATCAGCAATGGAGGTTGCTCTGCCAAGGCTGACTGTAAGAGAACCACCCCAGG
AAGGCGAGTGTGCACGTGCAAAGCAGGCTACACGGGTGATGGCATTGTGTGCCTGGAAATCAACCCGT
GTTTGGAGAACCATGGTGGCTGTGACAAGAATGCGGAGTGCACACAGACAGGACCCAACCAGGCTGCC
TGTAACTGTTTGCCAGCATACACTGGAGATGGAAAGGTCTGCACACTCATCAATGTCTGCTTAACTAA
AAATGGCGGCTGTAGTGAATTTGCCATCTGCAACCACACTGGGCAAGTAGAAAGGACTTGTACTTGCA
AGCCAAACTACATTGGAGATGGATTTACCTGCCGCGGCAGCATTTATCAGGAGCTTCCCAAGAACCCG
AAAACTTCCCAGTATTTCTTCCAGTTGCAGGAGCATTTCGTGAAAGATCTGGTCGGCCCAGGCCCCTT
CACTGTTTTTGCACCTTTATCTGCAGCCTTTGATGAGGAAGCTCGGGTTAAAGACTGGGACAAATACG
GTTTAATGCCCCAGGTTCTTCGGTACCATGTGGTCGCCTGCCACCAGCTGCTTCTGGAAAACCTGAAA
TTGATCTCAAATGCTACTTCCCTCCAAGGAGAGCCAATAGTCATCTCCGTCTCTCAGAGCACGGTGTA
TATAAATAATAAGGCTAAGATCATATCCAGTGATATCATCAGTACTAATGGGATTGTTCATATCATAG
ACAAATTGCTATCTCCCAAAAATTTGCTTATCACTCCCAAAGACAACTCTGGAAGAATTCTGCAAAAT
CTTACGACTTTGGCAACAAACAATGGCTACATCAAATTTAGCAACTTAATACAGGACTCAGGTTTGCT
GAGTGTCATCACCGATCCCATCCACACCCCAGTCACTCTCTTCTGGCCCACCGACCAAGCCCTCCATG
CCCTACCTGCTGAACAACAGGACTTCCTGTTCAACCAAGACAACAAGGACAAGCTGAAGGAGTATTTG
AAGTTTCATGTGATACGAGATGCCAAGGTTTTAGCTGTGGATCTTCCCACATCCACTGCCTGGAAGAC
CCTGCAAGGTTCAGAGCTGAGTGTGAAATGTGGAGCTGGCAGGGACATCGGTGACCTCTTTCTGAATG
GCCAAACCTGCAGAATTGTGCAGCGGGAGCTCTTGTTTGACCTGGGTGTGGCCTACGGCATTGACTGT
CTGCTGATTGATCCCACCCTGGGGGGCCGCTGTGACACCTTTACTACTTTCGATGCCTCGGGGGAGTG
TGGGAGCTGTGTCAATACTCCCAGCTGCCCAAGGTGGAGTAAACCAAAGGGTGTGAAGCAGAAGTGTC
TCTACAACCTGCCCTTCAAGAGGAACCTGGAAGGCTGCCGGGAGCGGTGCAGCCTGGTGATACAGATC
CCCAGGTGCTGCAAGGGCTACTTCGGGCGAGACTGTCAGGCCTGCCCTGGAGGACCAGATGCCCCGTG
TAATAACCGGGGTGTCTGCCTTGATCAGTACTCGGCCACCGGAGAGTGTAAATGCAACACCGGCTTCA
ATGGGACGGCGTGTGAGATGTGCTGGCCGGGGAGATTTGGGCCTGATTGTCTGCCCTGTGGCTGCTCA
GACCACGGACAGTGCGATGATGGCATCACGGGCTCCGGGCAGTGCCTCTGTGAAACGGGGTGGACAGG
CCCCTCGTGTGACACTCAGGCAGTTTTGCCTGCAGTGTGTACGCCTCCTTGTTCTGCTCATGCCACCT
GTAAGGAGAACAACACGTGTGAGTGTAACCTGGATTATGAAGGTGACGGAATCACATGCACAGTTGTG
GATTTCTGCAAACAGGACAACGGGGGCTGTGCAAAGGTGGCCAGATGCTCCCAGAAGGGCACGAAGGT
CTCCTGCAGCTGCCAGAAGGGATACAAAGGGGACGGGCACAGCTGCACAGAGATAGACCCCTGTGCAG
ACGGCCTTAACGGAGGGTGTCACGAGCACGCCACCTGTAAGATGACAGGCCCGGGCAAGCACAAGTGT
GAGTGTAAAAGTCACTATGTCGGAGATGGGCTGAACTGTGAGCCGGAGCAGCTGCCCATTGACCGCTG
CTTACAGGACAATGGGCAGTGCCATGCAGACGCCAAATGTGTCGACCTCCACTTCCAGGATACCACTG
TTGGGGTGTTCCATCTACGCTCCCCACTGGGCCAGTATAAGCTGACCTTTGACAAAGCCAGAGAGGCC
TGTGCCAACGAAGCTGCGACCATGGCAACCTACAACCAGCTCTCCTATGCCCAGAAGGCCAAGTACCA
CCTGTGCTCAGCAGGCTGGCTGGAGACCGGGCGGGTTGCCTACCCCACAGCCTTCGCCTCCCAGAACT
GTGGCTCTGGTGTGGTTGGGATAGTGGACTATGGACCTAGACCCAACAAGAGTGAAATGTGGGATGTC
TTCTGCTATCGGATGAAAGATGTGAACTGCACCTGCAAGGTGGGCTATGTGGGAGATGGCTTCTCATG
CAGTGGGAACCTGCTGCAGGTCCTGATGTCCTTCCCCTCACTCACAAACTTCCTGACGGAAGTGCTGG
CCTATTCCAACAGCTCAGCTCGAGGCCGTGCATTTCTAGAACACCTGACTGACCTGTCCATCCGCGGC
ACCCTCTTTGTGCCACAGAACAGTGGGCTGGGGGAGAATGAGACCTTGTCTGGGCGGGACATCGAGCA
CCACCTCGCCAATGTCAGCATGTTTTTCTACAATGACCTTGTCAATGGCACCACCCTGCAAACGAGGC
TGGGAAGCAAGCTGCTCATCACTGCCAGCCAGGACCCACTCCAACCGACGGAGACCAGGTTTGTTGAT
GGAAGAGCCATTCTGCAGTGGGACATCTTTGCCTCCAATGGGATCATTCATGTCATTTCCAGGCCTTT
AAAAGCACCCCCTGCCCCCGTGACCTTGACCCACACTGGCTTGGGAGCAGGGATCTTCTTTGCCATCA
TCCTGGTGACTGGGGCTGTTGCCTTGGCTGCTTACTCCTACTTTCGGATAAACCGGAGAACAATCGGC
TTCCAGCATTTTGAGTCGGAAGAGGACATTAATGTTGCAGCTCTTGGCAAGCAGCAGCCTGAGAATAT
CTCGAACCCCTTGTATGAGAGCACAACCTCAGCTCCCCCAGAACCTTCCTACGACCCCTTCACGGACT
CTGAAGAACGGCAGCTTGAGGGCAATGACCCCTTGAGGACACTGTGAGGGCCTG

| | | | |
|---|---|---|---|
| NOV9y, CG50736-24 | SEQ ID NO: 256 | 2551 aa | MW at 276991.0kD |
| Protein Sequence | | | |

MMLQHLVIFCLGLVVQNFCSPAETTGQARRCDRKSLLTIRTECRSCALNLGVKCPDGYTMITSGSVGV
RDCRYTFEVRTYSLSLPGCRHICRKDYLQPRCCPGRWGPDCIECPGGAGSPCNGRGSCAEGMEGNGTC
SCQEGFGGTACETCADDNLFGPSCSSVCNCVHGVCNSGLDGDGTCECYSAYTGPKCDKPIPECAALLC
PENSRCSPSTEDENKLECKCLPNYRGDGKYCDPINPCLRKICHPHAHCTYLGPNRHSCTCQEGYRGDG
QVCLPVDPCQINFGNCPTKSTVCKYDGPGQSHCECKEHYQNFVPGVGCSMTDICKSDNPCHRNANCTT
VAPGRTECICQKGYVGDGLTCYGNIMERLRELNTEPRGKWQGRLTSFISLLDKAYAWPLSKLGPFTVL
LPTDKGLKGFNVNELLVDNKAAQYFVKLHIIAGQMNIEYMNNTDMFYTLTGKSGEIFNSDKDNQIKLK
LHGGKKKVKIIQGDIIASNGLLHILDRAMDKLEHTFESNNEQTIMTMLQPRYSKFRSLLEETNLGHAL
DEDGVGGPYTIFVPNNEALNNMKDGTLDYLLSPEGSRKLLELVRYHIVPFTQLEVATLISTPHIRSMA
NQLIQFNTTDNGQILANDVAMEEIEITAKNGRIYTLTGVLIPPSIVPILPHRCDETKREMKLGTCVSC
SLVYWSRCPANSEPTALFTHRCVYSGRFGSLKSGCARYCNATVKIPKCCKGFYGPDCNQCPGGFSNPC
SGNGQCADSLGGNGTCICEEGFQGSQCQFCSDPNKYGPRCNKKCLCVHGTCNNRIDSDGACLTGTCRD
GSAGRLCDKQTSACGPYVQFCHIHATCEYSNGTASCICKAGYEGDGTLCSEMDPCTGLTPGGCSRNAE
CIKTGTGTHTCVCQQGWTGNGRDCSEINNCLLPSAGGCHDNASCLYVGPGQNECECKKGFRGNGIDCE
PITSCLEQTGKCHPLASCQSTSSGVWSCVCQEGYEGDGFLCYGNAAVELSFLSEAAIFNRWINNASLQ
PTLSATSNLTVLVPSQQATEDMDQDEKSFWLSQSNIPALIKYHMLLGTYRVADLQTLSSSDMLATSLQ
GNFLHLAKVDGNITIEGASIVDGDNAATNGVIHIINKVLVPQRRLTGSLPNLLMRLEQMPDYPIFRGY
IIQYNLANAIEAADAYTVFAPNNNAIENYIREKKVLSLEEDVLRYHVVLEEKLLKNDLHNGMHRETML
GFSYFLSFFLHNDQLYVNEAPINYTNVATDKGVIHGLGKVLEIQKNRCDNNATTIIRGRCRTCSSELT
CPFGTKSLGNEKRRCIYTSYFMGRRTLFIGCQPKCVRTVITRECCAGFFGPQCQPCPGNAQNVCFGNG
ICLDGVNGTGVCECGEGFSGTACETCTEGKYGIHCDQACSCVHGRCNQGPLGDGSCDCDVGWRGVHCD
NATTEDNCNGTCHTSANCLTNSDGTASCKCAAGFQGNGTICTAINACEISNGGCSAKADCKRTTPGRR
VCTCKAGYTGDGIVCLEINPCLENHGGCDKNAECTQTGPNQAACNCLPAYTGDGKVCTLINVCLTKNG
GCSEFAICNHTGQVERTCTCKPNYIGDGFTCRGSIYQELPKIJPKTSQYFFQLQEHFVDLVGPGPFTV
FAPLSAAFDEEARVKDWDKYGLMPQVLRYHVVACHQLLLENLKLISNATSLQGEPIVISVSQSTVYIN
NKAKIISSDIISTNGIVHIIDKLLSPKNLLITPKDNSGRILQNLTTLATNNGYIKFSNLIQDSGLLSV
ITDPIHTPVTLFWPTDQALHALPAEQQDFLFNQDNKDKLKEYLKFHVIRDAKVLAVDLPTSTAWKTLQ
GSELSVKCGAGRDIGDLFLNGQTCRIVQRELLFDLGVAYGIDCLLIDPTLGGRCDTFTTFDASGECGS
CVNTPSCPRWSKPKGVKQKCLYNLPFKRNLEGCRERCSLVIQIPRCCKGYFGRDCQACPGGPDAPCNN
RGVCLDQYSATGECKCNTGFNGTACEMCWPGRFGPDCLPCGCSDHGQCDDGITGSGQCLCETGWTGPS
CDTQAVLPAVCTPPCSAHATCKENNTCECNLDYEGDGITCTVVDFCKQDNGGCAKVARCSQKGTKVSC
SCQKGYKGDGHSCTEIDPCADGLNGGCHEHATCKMTGPGKHKCECKSHYVGDGLNCEPEQLPIDRCLQ
DNGQCHADAKCVDLHFQDTTVGVFHLRSPLGQYKLTFDKAREACANEAATMATYNQLSYAQKAXYHLC
SAGWLETGRVAYPTAFASQNCGSGVVGIVDYGPRPNKSEMWDVFCYRMKDVNCTCKVGYVGDGFSCSG
NLLQVLMSFPSLTNFLTEVLAYSNSSARGRAFLEHLTDLSIRGTLFVPQNSGLGENETLSGRDIEHHL
ANVSMFFYNDLVNGTTLQTRLGSKLLITASQDPLQPTETRFVDGRAILQWDIFASNGIIHVISRPLKA
PPAPVTLTRTGLGAGIFFAIILVTGAVALAAYSYFRINRRTIGFQHFESEEDINVAALGKQQPENISN
PLYESTTSAPPEPSYDPFTDSEERQLEGNDPLRTL

A ClustalW comparison of the above protein sequences yields the following sequence alignment shown in Table 9B.

TABLE 9B
Comparison of the NOV9 protein sequences.

| | |
|---|---|
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | MERQETGNSKTRYHATAIVQAKHDKGLNKNGTSGDEEQKIKVGDRDRENKGFDGLLDVWN |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | ------------------------------------------------------------ |
| NOV9x | ------------------------------------------------------------ |
| NOV9y | ------------------------------------------------------------ |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | TLNFIHPCFAVCNCVHGVCNSGLDGDGTCECYSAYTGPKCDKLTENFHTSHLTLWPVHDS |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | ------------------------------------------------------------ |
| NOV9x | ------------------------------------------------------------ |
| NOV9y | ------------------------------------------------------------ |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | KHWGSLRHQNMNGTCSSGGGKGDPDVYQNGLIFHGGGTSGGLSSSRNRRSSVKRPEKWKG |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | ------------------------------------------------------------ |
| NOV9x | ------------------------------------------------------------ |
| NOV9y | -----------------------------------------------MMLQHLVIFCLGL |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | DDRDGGGKEGQQRRRADTESSLQRGHIKTPLPHRQGEARITETTGNCVSAGMTGTNANHT |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | ------------------------------------------------------------ |
| NOV9x | ------------------------------------------------------------ |
| NOV9y | VVQNFCSPAETTGQARRCDRKSLLTIRTECRSCALNLGVKCPDGYTMITSGSVGVRDCRY |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | KVHPTVQSLTEYDSFQTHSTSRLKEFEKQQVKERFSDPPLMQAIKPSHEKYPPYAQRKGT |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | ------------------------------------------------------------ |
| NOV9x | ------------------------------------------------------------ |
| NOV9y | TFEVRTYSLSLPGCRRICRKDYLQPRCCPGRWGPDCIECPGGAGSPCNGRGSCAEGMEGN |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | SLSPKTQGHGDDEQALLSFLHSITLSLYLYPTTFFHDSPVFIKPGIKTLRLNHFFGSSFP |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | ------------------------------------------------------------ |
| NOV9x | ------------------------------------------------------------ |
| NOV9y | GTCSCQEGFGGTACETCADDNLFGPSCSSVCNCVHGVCNSGLDGDGTCECYSAYTGPKCD |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | YEGSSVIXXMGIEVWKNWCQNADTLAAAPAPSLNVQPCSAQKIPDVRLPLKMKTNWNANA |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | ------------------------------------------------------------ |
| NOV9x | ------------------------------------------------------------ |
| NOV9y | KPIPECAALLCPENSRCSPSTEDENKLECKCLPNYRGDGKYCDPINPCLRKTCHPHAHCT |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | FPITEAMANTATPSIHVYEKSATLMLIVRTWDQIGTVVHAKKATVGMAKCACLWTPAKLT |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | ------------------------------------------------------------ |
| NOV9x | ------------------------------------------------------------ |
| NOV9y | YLGPNPBSCTCQEGYRGDGQVCLPVDPCQINFGNCPTKSTVCKYDGPGQSHCECKEHYQN |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | LETALQSLQCANMMGLDRCICQKGYVGDGLTCYGNIMERLRELNTEPRGKWQGRLTSFIS |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | ------------------------------------------------------------ |
| NOV9x | ------------------------------------------------------------ |
| NOV9y | FVPGVGCSMTDICKSDNPCHRNANCTTVAPGRTECICQKGYVGDGLTCYGNIMERLRELN |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | LLESIQIVSVQLSEFSQREPTCVNTKSIASNLEGPLVPLSNHYPLQVNELLVDNKAAQYF |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | ------------------------------------------------------------ |
| NOV9x | ------------------------------------------------------------ |
| NOV9y | TEPRGKWQGRLTSFISLLDKAYAWPLSKLGPFTVLLPTDKGLKGFNVNELLVDNKAAQYF |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | VKLHIIAGQMNIEYMNNTDMFYTLTGKSGEIFNSDKDNQIKLKLHGGKKKVKIIQGDIIA |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | ------------------------------------------------------------ |
| NOV9x | ------------------------------------------------------------ |
| NOV9y | VKLHIIAGQMNIEYMNNTDMFYTLTGKSGEIFNSDKDNQIKLKLHGGKKKVKIIQGDIIA |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | SNGLLHILDRAMDKLEPTF-------------------ESNNEETNLGHALDEDGVGGPY |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | ------------------------------------------------------------ |
| NOV9x | ------------------------------------------------------------ |
| NOV9y | SNGLLHILDRAMDKLEHTFESNNEQTIMTMLQPRYSKFRSLLEETNLGHALDEDGVGGPY |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | TIFVPNNEALNNMKDGTLDYLLSP------------------ELEVATLISTPHIRSMAN |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | ------------------------------------------------------------ |
| NOV9x | ------------------------------------------------------------ |
| NOV9y | TIFVPNNEALNNMKDGTLDYLLSPEGSRKLLELVRYHIVPFTQLEVATLISTPHIRSMAN |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | QLIQFNTTDNGQILANDVAMEEIEITAKNGRIYTLTGVLIPPSIVPILPHRCDETKREMK |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | ------------------------------------------------------------ |
| NOV9x | ------------------------------------------------------------ |
| NOV9y | QLIQFNTTDNGQILANDVAMEEIEITAKNGRIYTLTGVLIPPSIVPILPHRCDETKREMK |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | LGTCVSCSLVYWSRCPANSEPTALFTHRCVYSGRFGSLKSGCARYC-------------- |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | ------------------------------------------------------------ |
| NOV9x | ------------------------------------------------------------ |
| NOV9y | LGTCVSCSLVYWSRCPANSEPTALFTHRCVYSGRFGSLKSGCARYCNATVKIPKCCKGFY |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | ---------------NATVKCADSLGGNGTCICEEGFQGSQCQFCSDPNKYGPRCNKKCL |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | ------------------------------------------------------------ |
| NOV9x | ------------------------------------------------------------ |
| NOV9y | GPDCNQCPGGFSNPCSGNGQCADSLGGNGTCICEEGFQGSQCQFCSDPNKYGPRCNKKCL |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | CVHGTCNNRIDSDGACLTGTCRDGSAGRLCDKQTSACGPYVQFCHIHATCEYSNGTASCI |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | ------------------------------------------------------------ |
| NOV9x | ------------------------------------------------------------ |
| NOV9y | CVHGTCNNRIDSDGACLTGTCRDGSAGRLCDKQTSACGPYVQFCHIHATCEYSNGTASCI |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | CKAGYEGDGTLCSEMDPCTGLTPGGCSRNAECIKTGTGTHTCVCQQGWTGNGRDCSEINN |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | ------------------------------------------------------------ |
| NOV9x | ------------------------------------------------------------ |
| NOV9y | CKAGYEGDGTLCSEMDPCTGLTPGGCSRNAECIKTGTGTHTCVCQQGWTGNGRDCSEINN |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | CLLPSAGGCHDNASCLYVGPGQNECECKKGFRGNGIDCEPITSCLEQTGKCHPLASCQST |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | ------------------------------------------------------------ |
| NOV9x | ------------------------------------------------------------ |
| NOV9y | CLLPSAGGCHDNASCLYVGPGQNECECKKGFRGNGIDCEPITSCLEQTGKCHPLASCQST |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | SSGVWSCVCQEGYEGDGFLCYGNAAVELSFLSEAAIFNRWINNASLQPTLSATSNLTVLV |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | ------------------------------------------------------------ |
| NOV9x | ------------------------------------------------------------ |
| NOV9y | SSGVWSCVCQEGYEGDGFLCYGNAAVELSFLSEAAIFNRWINNASLQPTLSATSNLTVLV |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | PSQQATEDMDQDEKSFWLSQSNIPALIKYHMLLGTYRVADLQTLSSSDMLATSLQGNFLH |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | --------MDQDEKSFWLSQSNIPALIKYHNLLGTYRVADLQTLSSSDMLATSLQGNFLH |
| NOV9x | --------MDQDEKSFWLSQSNIPALIKYHMLLGTYRVADLQTLSSSDMLATSLQGNFLH |
| NOV9y | PSQQATEDMDQDEKSFWLSQSNIPALIKYHMLLGTYRVADLQTLSSSDMLATSLQGNFLH |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | LAKVDGNITIEGASIVDGDNAATNGVIHIINKVLVPQRRLTGSLPNLLMRLEQMPDYSIF |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | LAKVDGNITIEGASIVDGDNAATNGVIHIINKVLVPQRRLTGSLPNLLMRLEQMPDYSIF |
| NOV9x | LAKVDGNITIEGASIVDGDNAATNGVIHIINKVLVPQRRLTGSLPNLLMRLEQMPDYSIF |
| NOV9y | LAKVDGNITIEGASIVDGDNAATNGVIHIINKVLVPQRRLTGSLPNLLMRLEQMPDYPIF |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | RGYIIQYNLANAIEAADAYTVFAPNNNAIENYIREKKVLSLEEDVLRYHVVLEEKLLKND |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | RGYIIQYNLANAIEAADAYTVFAPNNNAIENYIREKKVLSLEEDVLRYHVVLEEKLLKND |
| NOV9x | RGYIIQYNLANAIEAADAYTVFAPNNNAIENYIREKKVLSLEEDVLRYHVVLEEKLLKND |
| NOV9y | RGYIIQYNLANAIEAADAYTVFAPNNNAIENYIREKKVLSLEEDVLRYHVVLEEKLLKND |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | LHNGMHRETMLGFSYFLSFFLHNDQLYVNEAPINYTNVATDKGVIHGLGKVLEIQKNRCD |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | LHNGMHRETMLGFSYFLSFFLHNDQLYVNEAPINYTNVATDKGVIHGLGKVLEIQKNRCD |
| NOV9x | LHNGMHRETMLGFSYFLSFFLHNDQLYVNEAPINYTNVATDKGVIHGLGKVLEIQKNRCD |
| NOV9y | LHNGMHRETMLGFSYFLSFFLHNDQLYVNEAPINYTNVATDKGVIHGLGKVLEIQKNRCD |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | NNDTTIIRGRCRTCSSELTCPFGTKSLGNEKRRCIYTSYFMGRRTLFIGCQPKCVRTVIT |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | NNDTTIIRGRCRTCSSELTCPFGTKSLGNEKRRCIYTSYFMGRRTLFIGCQPKCVRTVIT |
| NOV9x | NNDTTIIRGRCRTCSSELTCPFGTKSLGNEKRRCIYTSYFMGRRTLFIGCQPKCVRTVIT |
| NOV9y | NNATTIIRGRCRTCSSELTCPFGTKSLGNEKRRCIYTSYFMGRRTLFIGCQPKCVRTVIT |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | RECCAGFFGPQCQPCPGNAQNVCFGNGICLDGVNGTGVCECGEGFSGTACETCTEGKYGI |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | RECCAGFFGPQCQPCPGNAQNVCFGNGICLDGVNGTGVCECGEGFSGTACETCTEGKYGI |
| NOV9x | RECCAGFFGPQCQPCPGNAQNVCFGNGICLDGVNGTGVCECGEGFSGTACETCTEGKYGI |
| NOV9y | RECCAGFFGPQCQPCPGNAQNVCFGNGICLDGVNGTGVCECGEGFSGTACETCTEGKYGI |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | HCDQACSCVHGRCNQGPLGDGSCDCDVGWRGVHCDNATTEDNCNGTCHTSANCLTNSDGT |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | HCDQACSCVHGRCNQGPLGDGSCDCDVGWRGVHCDNATTEDNCNGTCHTSANCLTNSDGT |
| NOV9x | HCDQACSCVHGRCNQGPLGDGSCDCDVGWRGVHCDNATTEDNCNGTCHTSANCLTNSDGT |
| NOV9y | HCDQACSCVHGRCNQGPLGDGSCDCDVGWRGVHCDNATTEDNCNGTCHTSANCLTNSDGT |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | ASCKCAAGFQGNGTICTAINACEISNGGCSAXADCKRTTPGRRVCTCKAGYTGDGIVCLE |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | ASCKCAAGFQGNGTICTAINACEISNGGCSAXADCKRTTPGRRVCTCKAGYTGDGIVCLE |
| NOV9x | ASCKCAAGFQGNGTICTAINACEISNGGCSAKADCKRTTPGRRVCTCKAGYTGDGIVCLE |
| NOV9y | ASCKCAAGFQGNGTICTAINACEISNGGCSAKADCKRTTPGRRVCTCKAGYTGDGIVCLE |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | INPCLENHGGCDKNAECTQTGPNQAACNCLPAYTGDGKVCTLINVCLTKNGGCSEFAICN |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | INPCLENHGGCDKNAECTQTGPNQAACNCLPAYTGDGKVCTLINVCLTKNGGCGEFAICN |
| NOV9x | INPCLENHGGCDKNAECTQTGPNQAACNCLPAYTGDGKVCTLINVCLTKNGGCGEFAICN |
| NOV9y | INPCLENHGGCDKNAECTQTGPNQAACNCLPAYTGDGKVCTLINVCLTKNGGCSEFAICN |
| NOV9a | ------------------------------------------------------------ |
| NOV9b | ------------------------------------------------------------ |
| NOV9c | ------------------------------------------------------------ |
| NOV9d | ------------------------------------------------------------ |
| NOV9e | ----MEARKELESLPPFCLNSLHSIILTTKLTSQSLGGPRGVEERNEDREAKWHIAAKDS |
| NOV9f | ----MEARKELESLPPFCLNSLHSIILTTKLTSQSLGGPRGVEERMEDRRAKWHIAAKDS |
| NOV9g | ------------------------------------------------------------ |
| NOV9h | ----MEARKELESLPPFCLNSLHSIILTTKLTSQSLGGPRGVEERNEDREAKWHIAAKDS |
| NOV9i | ----MEARKELESLPPFCLNSLHSIILTTKLTSQSLGGPRGVEERMEDRPAKWHIAAKDS |
| NOV9j | ------------------------------------------------------------ |
| NOV9k | HTGQVERTCTCKPNYIGDGFTCRGSIYQELPKNPKTSQYFFQLQEHFVKDLVGPGPFTVF |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9g | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | HTGQVERTCTCKPNYIGDGFTCRGSIYQELPKNPKTSQYFFQLQEHFVKDLVGPGPFTVF |
| NOV9x | HTGQVERTCTCKPNYIGDGFTCRGSIYQELPKNPKTSQYFFQLQEHFVKDLVGPGPFTVF |
| NOV9y | HTGQVERTCTCKPNYIGDGFTCRGSIYQELFKNPKTSQYFFQLQEHFVKDLVGPGPFTVF |
| NOV9a | ---------------------MPQVLRYHVVACHQLLLENLKLISNATSLQGEPIVISVS |
| NOV9b | ------------------------------------------------------------ |
| NOV9c | ------------------------------------------------------------ |
| NOV9d | ------------------------------------------------------------ |
| NOV9e | CLWLKPSDLLLQVKDWDKYGLMPQVLRYHVVACHQLLLENLKLISNATSLQGEPIVISVS |
| NOV9f | CLWLKPSDLLLQVKDWDKYGLMPQVLRYHVVACHQLLLENLKLISNATSLQGEPIVISVS |
| NOV9g | ------------------------------------------------------------ |
| NOV9h | CLWLKPSDLLLQVKDWDKYGLMPQVLRYHVVACHQLLLENLKLISNATSLQGEPIVISVS |
| NOV9i | CLWLKPSDLLLQVKDWDKYGLMPQVLRYHVVACHQLLLENLKLISNATSLQGEPIVISVS |
| NOV9j | ------------------------VLRYHVVACHQLLLENLKLISNATSLQGEPIVISVS |
| NOV9k | APLSAAFDEEARVKDWDKYGLMPQVLRYHVVACHQLLLENLKLISNATSLQGEPIVISVS |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | APLSAAFDEEARVKDWDKYGLMPQVLRYHVVACHQLLLENLKLISNATSLQGEPIVISVS |
| NOV9x | APLSAAFDEEARVKDWDKYGLMPQVLRYHVVACHQLLLENLKLISNATSLQGEPIVISVS |
| NOV9y | APLSAAFDEEARVKDWDKYGLMPQVLRYHVVACHQLLLENLKLISNATSLQGEPIVISVS |
| NOV9a | QSTVYINNKAKIISSDIISTNGIVHIIDKLLSPKNLLITPKDNSGRILQNLTTLATNNGY |
| NOV9b | -------------------------------------------GSRILQNLTTLATNNGY |
| NOV9c | ------------------------------------------------------------ |
| NOV9d | ------------------------------------------------------------ |
| NOV9e | QSTVYINNKAKIISSDIISTNGIVHIIDKLLSPKNLLITPKDNSGRILQNLTTLATNNGY |
| NOV9f | QSTVYINNKAKIISSDIISTNGIVHIIDKLLSPKNLLITPKDNSGRILQNLTTLATNNGY |
| NOV9g | ------------------------------------------------------------ |
| NOV9h | QSTVYINNKAKIISSDIISTNGIVHIIDKLLSPKNLLITPKDNSGRILQNLTTLATNNGY |
| NOV9i | QSTVYINNKAKIISSDIISTNGIVHIIDKLLSPKNLLITPKDNSGRILQNLTTLATNNGY |
| NOV9j | QSTVYINNKAKIISSDIISTNGIVHIIDKLLSPKNLLITPKDNSGRILQNLTTLATNNGY |
| NOV9k | QSTVYINNKAKIISSDIISTNGIVHIIDKLLSPKNLLITPKDNSGRILQNLTTLATNNGY |
| NOV9l | ---------------------------------------------RILQNLTTLATNNGY |
| NOV9m | ---------------------------------------------RILQNLTTLATNNGY |
| NOV9n | ---------------------------------------------RILQNLTTLATNNGY |
| NOV9o | ---------------------------------------------RILQNLTTLATNNGY |
| NOV9p | ---------------------------------------------RILQNLTTLATNNGY |
| NOV9q | ---------------------------------------------RILQNLTTLATNNGY |
| NOV9r | ---------------------------------------------RILQNLTTLATNNGY |
| NOV9s | ---------------------------------------------RILQNLTTLATNNGY |
| NOV9t | ---------------------------------------------RILQNLTTLATNNGY |
| NOV9u | ---------------------------------------------RILQNLTTLATNNGY |
| NOV9v | ---------------------------------------------RILQNLTTLATNNGY |
| NOV9w | QSTVYINNKAKIISSDIISTNGIVHIIDKLLSPKNLLITPKDNSGRILQNLTTLATNNGY |
| NOV9x | QSTVYINNKAKIISSDIISTNGIVHIIDKLLSPKNLLITPKDNSGRILQNLTTLATNNGY |
| NOV9y | QSTVYINNKAKIISSDIISTNGIVHIIDKLLSPKNLLITPKDNSGRILQNLTTLATNNGY |
| NOV9a | IKFSNLIQDSGLLSVITDPIHTPVTLFWPTDQALHALPAE---QQDFLFNQDNKDKLKEY |
| NOV9b | IKFSNLIQDSGLLSVITDPIHTPVTLFWPTDQALHALPAE---QQDFLFNQDNKDKLKEY |
| NOV9c | ------------------------------------------------------------ |
| NOV9d | ------------------------------------------------------------ |
| NOV9e | IKFSNLIQDSGLLSVITDPIHTPVTLFWPTDQALHALPAE---QQDFLFNQDNKDKLKEY |
| NOV9f | IKFSNLIQDSGLLSVITDPIHTPVTLFWPTDQALHALPAE---QQDFLFNQDNKDKLKEY |
| NOV9g | ------------------------------------------------------------ |
| NOV9h | IKFSNLIQDSGLLSVITDPIHTPVTLFWPTDQALHALPAE---QQDFLFNQDNKDKLKEY |
| NOV9i | IKFSNLIQDSGLLSVITDPIHTPVTLFWPTDQALHALPAE---QQDFLFNQDNKDKLKEY |
| NOV9j | IKFSNLIQDSGLLSVITDPIHTPVTLFWPTDQALHALPAE---QQDFLFNQDNKDKLKEY |
| NOV9k | IKFSNLIQDSGLLSVITDPIHTPVTLFWPTDQALHALHALPAEQQDFLFNQDNKDKLKEY |
| NOV9l | IKFSNLIQDSGLLSVITDPIHTPVTLFWPTDQALHALPAE---QQDFLFNQDNKDKLKEY |
| NOV9m | IKFSNLIQDSGLLSVITDPIHTPVTLFWPTDQALHALPAE---QQDFLFNQDNKDKLKEY |
| NOV9n | IKFSNLIQDSGLLSVITDPIHTPVTLFWPTDQALHALPAE---QQDFLFNQDNKDKLKEY |
| NOV9o | IKFSNLIQDSGLLSVITDPIHTPVTLFWPTDQALHALPAE---QQDFLFNQDNKDKLKEY |
| NOV9p | IKFSNLIQDSGLLSVITDPIHTPVTLFWPTDQALHALPAE---QQDFLFNQDNKDKLKEY |
| NOV9q | IKFSNLIQDSGLLSVITDPIHTPVTLFWPTDQALHALPAE---QQDFLFNQDNKDKLKEY |
| NOV9r | IKFSNLIQDSGLLSVITDPIHTPVTLFWPTDQALHALPAE---QQDFLFNQDNKDKLKEY |
| NOV9s | IKFSNLIQDSGLLSVITDPIHTPVTLFWPTDQALHALPAE---QQDFLFNQDNKDKLKEY |
| NOV9t | IKFSNLIQDSGLLSVITDPIHTPVTLFWPTDQALHALPAE---QQDFLFNQDNKDKLKEY |
| NOV9u | IKFSNLIQDSGLLSVVTDPIHTPVTLFWPTDQALHALPAE---QQDFLFNQDNKDKLKEY |
| NOV9v | IKFSNLIQDSGLLSVITDPIHTPVTLFWPTDQALHALPAE---QQDFLFNQDNKDKLKEY |
| NOV9w | IKFSNLIQDSGLLSVITDPIHTPVTLFWPTDQALHALPAE---QQDFLFNQDNKDKLKEY |
| NOV9x | IKFSNLIQDSGLLSVITDPIHTPVTLFWPTDQALHALPAE---QQDFLFNQDNKDKLKEY |
| NOV9y | IKFSNLIQDSGLLSVITDPIHTPVTLFWPTDQALHALPAE---QQDFLFNQDNKDKLKEY |
| NOV9a | LKFHVIRDAKVLAVDLPTSTAWKTLQGSELSVKCGAGRDIGDLFLNGQTCRIVQRELLFD |
| NOV9b | LKFHVIRDAKVLAVDLPTSTAWKTLQGSELSVKCGAGRDIGDLFLNGQTCRIVQRELLFD |
| NOV9c | ------------------------------------------------------------ |
| NOV9d | ------------------------------------------------------------ |
| NOV9e | LKFHVIRDAKVLAVDLPTSTAWKTLQGSELSVKCGAGRDIGDLFLNGQTCRIVQRELLFD |
| NOV9f | LKFHVIRDAKVLAVDLPTSTAWKTLQGSELSVKCGAGRDIGDLFLNGQTCRIVQRELLFD |
| NOV9g | ---------------LPTSTAWKTLQGSELSVKCGAGRDIGDLFLNGQTCRIVQRELLFD |
| NOV9h | LKFHVIRDAKVLAVDLPTSTAWKTLQGSELSVKCGAGRDIGDLFLNGQTCRIVQRELLFD |
| NOV9i | LKFHVIRDAKVLAVDLPTSTAWKTLQGSELSVKCGAGRDIGDLFLNGQTCRIVQRELLFD |
| NOV9j | LKFHVIRDAKVLAVDLPTSTAWKTLQGSELSVKCGAGRDIGDLFLNGQTCRIVQRELLFD |
| NOV9k | LKFHVIRDAKVLAVDLPTSTAWKTLQGSELSVKCGAGRDIGDLFLNGQTYRIVQRELLFD |
| NOV9l | LKFHVIRDAKVLAVDLPTSTAWKTLQGSELSVKCGAGRDIGDLFLNGQTCRIVQRELLFD |
| NOV9m | LKFHVIRDAKVLAVDLPTSTAWKTLQGSELSVKCGAGRDIGDLFLNGQTCRIVQRELLFD |
| NOV9n | LKFHVIRDAKVLAVDLPTSTAWKTLQGSELSVKCGAGRDIGDLFLNGQTCRIVQRELLFD |
| NOV9o | LKFHVIRDAKVLAVDLPTSTAWKTLQGSELSVKCGAGRDIGDLFLNGQTCRIVQRELLFD |
| NOV9p | LKFHVIRDAKVLAVDLPTSTAWKTLQGSELSVKCGAGRDIGDLFLNGQTCRIVQRELLFD |
| NOV9q | LKFHVIRDAKVLAVDLPTSTAWKTLQGSELSVKCGAGRDIGDLFLNGQTCRIVQRELLFD |
| NOV9r | LKFHVIRDAKVLAVDLPTSTAWKTLQGSELSVKCGAGRDIGDLFLNGQTCRIVQRELLFD |
| NOv9s | LKFHVIRDAKVLAVDLPTSTAWKTLQGSELSVKCGAGRDIGDLFLNGQTCRIVQRELLFD |
| NOV9t | LKFHVIRDAKVLAVDLPTSTAWKTLQGSELSVKCGAGRDIGDLFLNGQTCRIVQRELLFD |
| NOV9u | LKFHVIRDAKVLAVDLPTSTAWKTLQGSELSVKCGAGRDIGDLFLNGQTCRIVQRELLFD |
| NOV9v | LKFHVIRDAKVLAVDLPTSTAWKTLQGSELSVKCGAGRDIGDLFLNGQTCRIVQRELLFD |
| NOV9w | LKFHVIRDAKVLAVDLPTSTAWKTLQGSELSVKCGAGRDIGDLFLNGQTCRIVQRELLFD |
| NOV9x | LKFHVIRDAKVLAVDLPTSTAWKTLQGSELSVKCGAGRDIGDLFLNGQTCRIVQRELLFD |
| NOV9y | LKFHVIRDAKVLAVDLPTSTAWKTLQGSELSVKCGAGRDIGDLFLNGQTCRIVQRELLFD |
| NOV9a | LGVAYGIDCLLIDPTLGGRCDTFTTFDASGECGSCVNTPSCPRWSKPKGVKQKCLYNLPF |
| NOV9b | LGVAYGIDCLLIDPTLGGRCDTFTTFDASGECGSCVNTPSCPRWSKPKGVKQKCLYNLPF |
| NOV9c | ------------------------------------------------------------ |
| NOV9d | ------------------------------------------------------------ |
| NOV9e | LGVAYGIDCLLIDPTLGGRCDTFTTFDASGECGSCVNTPSCPRWSKPKGVKQKCLYNLPF |
| NOV9f | LGVAYGIDCLLIDPTLGGRCDTFTTFDASGECGSCVNTPSCPRWSKPKGVKQKCLYNLPF |
| NOV9g | LGVAYGIDCLLIDPTLGGRCDTFTTFDASGECGSCVNTPSCPRWSKPKGVKQKCLYNLPF |
| NOV9h | LGVAYGIDCLLIDPTLGGRCDTFTTFDASGECGSCVNTPSCPRWSKPKGVKQKCLYNLPF |
| NOV9i | LGVAYGIDCLLIDPTLGGRCDTFTTFDASGECGSCVNTPSCPRWSKPKGVKQKCLYNLPF |
| NOV9j | LGVAYGIDCLLIDPTLGGRCDTFTTFDASGECGSCVNTPSCPRWSKPKGVKQKCLYNLPF |
| NOV9k | LGVAYGIDCLLIDPTLGGRCDTFTTFDASGECGSCVNTPSCPRWSKPKGVKQKCLYNLPF |
| NOV9l | LGVAYGIDCLLIDPTLGGRCDTFTTFDAS-------------------GVKQKCLYNLPF |
| NOV9m | LGVAYGIDCLLIDPTLGGRCDTFTTFDAS-------------------GVKQKCLYNLPF |
| NOV9n | LGVAYGIDCLLIDPTLGGRCDTFTTFDASGECGSCVNTPSCPRWSKPKGVKQKCLYNLPF |
| NOV9o | LGVAYGIDCLLIDPTLGGRCDTFTTFDASGECGSCVNTPSCPRWSKPKGVKQKCLYNLPF |
| NOV9p | LGVAYGIDCLLIDPTLGGRCDTFTTFDASGECGSCVNTPSCPRWSKPKGVKQKCLYNLPF |
| NOV9q | LGVAYGIDCLLIDPTLGGRCDTFTTFDASGECGSCVNTPSCPRWSKPKGVKQKCLYNLPF |
| NOV9r | LGVAYGIDCLLIDPTLGGRCDTFTTFDASGECGSCVNTPSCPRWSKPKGVKQKCLYNLPF |
| NOV9s | LGVAYGIDCLLIDPTLGGRCDTFTTFDASGECGSCVNTPSCPRWSKPKGVKQRCLYNLPF |
| NOV9t | LGVACGIDCLLIDPTLGGRCDTFTTFDASGECGSCVNTPSCPRWSKPKGVKQKCLYNLPF |
| NOV9u | LGVAYGIDCLLIDPTLGGRCDTFTTFDASGECGSCVNTPSCPRWSKPKGVKQKCLYNLPF |
| NOV9v | LGVAYGIDCLLIDPTLGGRCDTFTTFDASGECGSCVNTPSCPRWSKPKGVKQKCLYNLPF |
| NOV9w | LGVAYGIDCLLIDPTLGGRCDTFTTFDASGECGSCVNTPSCPRWSKPKGVKQKCLYNLPF |
| NOV9x | LGVAYGIDCLLIDPTLGGRCDTFTTFDASGECGSCVNTPSCPRWSKPKGVKQKCLYNLPF |
| NOV9y | LGVAYGIDCLLIDPTLGGRCDTFTTFDASGECGSCVNTPSCPRWSKPKGVKQKCLYNLPF |
| NOV9a | KRNLEGCRERCSLVIQIPRCCKGYFGRDCQACPGGPDAPCNNRGVCLDQYSATGECKCNT |
| NOV9b | KRNLEGCRERCSLVIQIPRCCKGYFGRDCQACPGGPDAPCNNRGVCLDQYSATGECKCNT |
| NOV9c | ------------------------------------------------------------ |
| NOV9d | ------------------------------------------------------------ |
| NOV9e | KRNLEGCRERCSLVIQIPRCCKGYFGRDCQACPGGPVAPCNNRGVCLDQYSATGECKCNT |
| NOV9f | KRNLEGCRERCSLVIQIPRCCKGYFGRDCQACPGGPDAPCNNRGVCLDQYSATGECKCNT |
| NOV9g | KRNLEGCRERCSLVIQIPRCCKGYFGRDCQACPGGPDAPCNNRGVCLDQYSATGECKCNT |
| NOV9h | KRNLEGCRERCSLVIQIPRCCKGYFGRDCQGE--GASSP-------LATL----KVSALI |
| NOV9i | KRNLEGCRERCSLVIQIP------------------------------------------ |
| NOV9j | KRNLEGCRERCSLVIQIPRCCKGYFGRDCQACPGGPDAPCNNRGVCLDQYSATGECKCNT |
| NOV9k | KRNLEGCRERCSLVIQIPRCCKGYFGRDCQACPGGPDAPCNNRGVCLDQYSATGECKCNT |
| NOV9l | KRNLEGCRERCSLVIQIPRCCKGYFGRDCQACPGGPDAPCNNRGVCLDQYSATGECKCNT |
| NOV9m | KRNLEGCRERCSLVIQIPRCCKGYFGRDCQACPGGPDAPCNNRGVCLDQYSATGECKCNT |
| NOV9n | KRNLEGCRERCSLVIQIPRCCKGYFGRDCQACPGGPDAPCNNRGVCLDQYSATGECKCNT |
| NOV9o | KRNLEGCRERCSLVIQIPRCCKGYFGRDCQACPGGPDAPCNNRGVCLDLYSATGECKCNT |
| NOV9p | KPNLEGCRERCSLVTQIPRCCKGYFGRDCQACPGGPDAPCNNRGVCLDQYSATGECKCNT |
| NOV9q | KRNLEGCRERCSLVIQIPRCCKGYFGRDCQACPGGPDAPCNNRGVCLDQYSATGECKCNT |
| NOV9r | KRNLEGCRERCSLVIQIPRCCKGYFGRDCQACPGGPDAPCNNRGVCLDQYSATGECKCNT |
| NOV9s | KRNLEGCRERCSLVIQIPRCCKGYFGRDCQACPGGPDAPCNNRGVCLDQYSATGECKCNT |
| NOV9t | KRNLEGCRERCSLVIQIPRCCKGYFGRDCQACPGGPDAPCNNRGVCLDQYSATGECKCNT |
| NOV9u | KRNLEGCRERCSLVIQIPRCCKGYFGRDCQACPGGPDAPCNNRGVCLDQYSATGECKCNT |
| NOV9v | KRNLEGCRERCSLVIQIPRCCKGYFGRDCQACPGGPDAPCNNRGVCLDQYSATGECKCNT |
| NOV9w | KRNLEGCRERCSLVIQIPRCCKGYFGRDCQPR--------NSKSVCLDQYSATGECKCNT |
| NOV9x | KRNLEGCRERCSLVIQIPRCCKGYFGRDCQACPGGPDAPCNNRGVCLDQYSATGECKCNT |
| NOV9y | KRNLEGCRERCSLVIQIPRCCKGYFGRDCQACPGGPDAPCNNRGVCLDQYSATGECKCNT |
| NOV9a | GFNGTACEMCWPGRFGPDCLPCGCSDHGQCDDGITGSGQCLCETGWTGPSCDTQAVLPAV |
| NOV9b | GFNGTACEMCWPGRFGPDCLPCGCSDHGQCDDGITGSGQCLCETGWTGPSCDTQAVLPAV |
| NOV9c | --------MCWPGRFGPDCLPCGCSDHGQCDDGITGSGQCLCETGWTGPSCDTQAVLSAV |
| NOV9d | --------MCWPGRFGPDCLPCGCSDHGQCDDGITGSGQCLCETGWTGPSCDTQAVLSAV |
| NOV9e | GFNGTACEMCWPGRFGPDCLPCGCSDHGQCDDGITGSGQCLCETGWTGPSCDTQAVLSAV |
| NOV9f | GFNGTACEMCWPGRFGPDCLPCGCSDHGQCDDGITGSGQCLCETGWTGPSCDTQAVLSAV |
| NOV9g | GFNGTACEMCWPGRFGPDCLPCGCSDHGQCDDGITGSGQCLCETGWTGPSCDTQAVLSAV |
| NOV9h | STRPPESVNATPASMGRRVRCAGRGDLGLIVCPVA-A--------------QTTDSAMMA |
| NOV9i | ---------SLPWRTRCPV----------------------------------------- |
| NOV9j | GFNGTACEMCWPGRFGPDCLPCGCSDHGQCDDGITGSGQCLCETGWTGPSCDTQAVLSAV |
| NOV9k | GFNGTACEMCWPGRFGPDCLPCGCSDHGQCDDGITGSGQCLCETGWTGPSCDTQAVLPAV |
| NOV9l | GFNGTACEMCWPGRFGPDCLPCGCSDHGQCDDGITGSGQCLCETGWTGPSCDTQAVLPAV |
| NOV9m | GFNGTACEMCWPGRFGPDCLPCGCSDHGQCDDGITGSGQCLCETGWTGPSCDTQAVLPAV |
| NOV9n | GFNGTACEMCWPGRFGPDCLPCGCSDHGQCNDGITGSGQCLCETGWTGPSCDTQAVLPAV |
| NOV9o | GFNGTACEMCWPGRFGPDCLPCGCSDHGQCDDGITGSGQCLCETGWTGPSCDTQAVLPAV |
| NOV9p | GFNGTACEMCWPGRFGPDCLPCGCSDHGQCDDGITGSGQCLCETGWTGPSCDTQAVLPAV |
| NOV9q | GFNGTACEMCWPGRFGPDCLPCGCSDHGQCDDGITGSGQCLCETGWTGPSCDTQAVLPAV |
| NOV9r | GFNGTACEMCWPGRFGPDCLPCGCSDHGQCDDGITGSGQCLCETGWTGPSCDTQAVLPAV |
| NOV9s | GFNGTACEMCWPGRFGPDCLPCGCSDHGQCDDGITGSGQCLCETGWTGPSCDTQAVLPAV |
| NOV9t | GFNGTACEMCWPGRFGPDCLPCGCSDHGQCDDGITGSGQCLCETGWTGPSCDTQAVLPAV |
| NOV9u | GFNGTACEMCWPGRFGPDCLPCGCSDHGQCDDGITGSGQCLCETGWTGPSCDTQAVLPAV |
| NOV9v | GFNGTACEMCWPGRFGPDCLPCGCSDHGQCDDGITGSGQCLCETGWTGPSCDTQAVLPAV |
| NOV9w | GFNGTACEMCWPGRFGPDCLPCGCSDHGQCDDGITGSGQCLCETGWTGPSCDTQAVLPAV |
| NOV9x | GFNGTACEMCWPGRFGPDCLPCGCSDHGQCDDGITGSGQCLCETGWTGPSCDTQAVLPAV |
| NOV9y | GFNGTACEMCWPGRFGPDCLPCGCSDHGQCDDGITGSGQCLCETGWTGPSCDTQAVLPAV |
| NOV9a | CTPPCSAHATCKENNTCECNLDYEGDGITCTVVDFCKQDNGGCAKVARCSQKGTKVSCSC |
| NOV9b | CTPPCSAHATCKENNTCECNLDYEGDGITCTVVDFCKQDNGGCAKVARCSQKGTKVSCSC |
| NOV9c | CTPPCSAHATCKENNTCECNLDYEGDGITCTVVDFCKQDNGGCAKVARCSQKGTKVSCSC |
| NOV9d | CTPPCSAHATCKENNTCECNLDYEGDGITCTVVDFCKQDNGGCAKVARCSQKGTKVSCSC |
| NOV9e | CTPPCSAHATCKENNTCECNLDYEGDGITCTVVDFCKQDNGGCAKVARCSQKGTKVSCSC |
| NOV9f | CTPPCSAHATCKENNTCECNLDYEGDGITCTVVDFCKQDNGGCAKVARCSQKGTKVSCSC |
| NOV9g | CTPPCSAHATCKENNTCECNLDYEGDGITCTVVDFCKQDNGGCAKVARCSQKGTKVSCSC |
| NOV9h | SRAPGSASVKRG------------GQAPRVTLRQFCLQCVRLLVLLMPPVRRTTRVSVTW |
| NOV9i | ------------------------------------------------------------ |
| NOV9j | CTPPCSAHATCKENNTCECNLDYEGDGITCTVVDFCKQDNGGCAKVARCSQKGTKVSCSC |
| NOV9k | CTPPCSAHATCKENNTCECNLDYEGDGITCTVVDFCKQDNGGCAKVARCSQKGTKVSCSC |
| NOV9l | CTPPCSAHATCKENNTCECNLDYEGDGITCTVVDFCKQDNGGCAKVARCSQKGTKVSCSC |
| NOV9m | CTPPCSAHATCKENNTCECNLDYEGDGITCTVVDFCKQDNGGCAKVARCSQKGTKVSCSC |
| NOV9n | CTPPCSAHATCKENNTCECNLDYEGDGITCTVVDFCKQDNGGCAKVARCSQKGTKVSCSC |
| NOV9o | CTPPCSAHATCKENNTCECNLDYEGDGITCTVVDFCKQDNGGCAKVARCSQKGTKVSCSC |
| NOV9p | CTPPCSAHATCKENNTCECNLDYEGDGITCTVVDFCKQDNGGCAKVARCSQKGTKVSCSC |
| NOV9q | CTPPCSAHATCKENNTCECYLDYEGDGITCTVVDFCKQDNGGCAKVARCSQKGTKVSCSC |
| NOV9r | CTPPCSAHATCKENNTCECNLDYEGDGITCTVVDFCKQDNGGCAKVARCSQKGTKVSCNC |
| NOV9s | CTPPCSAHATCKENNTCECNLDYEGDGITCTVVDFCKQDNGGCAKVARCSQKGTKVSCSC |
| NOV9t | CTPPCSAHATCKENNTCECNLDYEGDGITCTVVDFCKQDNGGCAKVARCSQKGTKVSCSC |
| NOV9u | CTPPCSAHATCKENNTCECNLDYEGDGITCTVVDFCKQDNGGCAKVARCSQKGTKVSCSC |
| NOV9v | CTPPCSAHATCKENNTCECNLDYEGDGITCTVVDFCKQDNGGCAKVARCSQKGTKVSCSC |
| NOV9w | CTPPCSAHATCKENNTCECNLDYEGDGITCTVVDFCKQDNGGCAKVARCSQKGTKVSCSC |
| NOV9x | CTPPCSAHATCKENNTCECNLDYEGDGITCTVVDFCKQDNGGCAKVARCSQKGTKVSCSC |
| NOV9y | CTPPCSAHATCKENNTCECNLDYEGDGITCTVVDFCKQDNGGCAKVARCSQKGTKVSCSC |
| NOV9a | QKGYKGDGHSCTEIDPCADGLNGGCHEHATCKMTGPGKHKCECKSHYVGDGLNCEPEQLP |
| NOV9b | QKGYKGDGHSCTEIDPCADGLNGGCHEHATCKMTGPGKHKCECKSHYVGDGLNCGPEQLP |
| NOV9c | QKGYKGDGHSCTEIDPCADGLNGGCHEHATCKMTGPGKHKCECKSHYVGDGLNCEPEQLP |
| NOV9d | QKGYKGDGHSCTEIDPCADGLNGGCHEHATCKMTGPGKHKCECKSHYVGDGLNCEPEQLP |
| NOV9e | QKGYKGDGHSCTEIDPCADGLNGGCHEHATCKMTGPGKHKCECKSHYVGDGLNCEPEQLP |
| NOV9f | QKGYKGDGHSCTEIDPCADGLNGGCHEHATCKMTGPGKHKCECKSHYVGDGLNCEPEQLP |
| NOV9g | QKGYKGDGHSCTEIDPCADGLNGGCHEHATCKMTGPGKHKCECKSHYVGDGLNCEPEQLP |
| NOV9h | IMKVT---ESHAQLWISANRTTGAVQRWPDAPRPAR-RSPAAARRDTKGTGTAAQR---- |
| NOV9i | ------------------------------------------------------------ |
| NOV9j | QKGYKGDGHSCTEIDPCADGLNGGCHEHATCKMTGPGKHKCECKSHYVGDGLNCEPEQLP |
| NOV9k | QKGYKGDGHSCTEIDPCADGLNGGCHEHATCKMTGPGKHKCECKSHYVGDGLNCEPEQLP |
| NOV9l | QKGYKGDGHSCTEIDPCADGLNGGCHEHATCKMTGPGKHKCECKSHYVGDGLNCEPEQLP |
| NOV9m | QKGYKGDGHSCTEIDPCADGLNGGCHEHATCKMTGPGKHKCECKSHYVGDGLNCEPEQLP |
| NOV9n | QKGYKGDGHSCTEIDPCADGLNGGCHEHATCKMTGPGKHKCECKSHYVGDGLNCEPEQLP |
| NOV9o | QKGYKGDGHSCTEIDPCADGLNGGCHEHATCKMTGPGKHKCECKSHYVGDGLNCEPEQLP |
| NOV9p | QKGYKGDGHSCTEIDPCADGLNGGCHEHATCKNTGPGKHKCECKSHYVGDGLNCEPEQLP |
| NOV9q | QKGYKGDGHSCTEIDPCADGLNGGCHEHATRKMTGPGKHKCECKSHYVGDGLNCEPEQLP |
| NOV9r | QKGYKGDGHSCTEIDPCADGLNGGCHEHATCKMTGPGKHKCECKSHYVGDGLNCEPEQLP |
| NOV9s | QKGYKGDGHSCTEIDPCADGLNGGCHEHATCKMTGPGKHKCECKSHYVGDGLNCEPEQLP |
| NOV9t | QKGYKGDGHSCTEIDPCADGLNGGCHEHATCKNTGPGKHKCECKSHYVGDGLNCEPEQLP |
| NOV9u | QKGYKGDGHSCTETDPCADGLNGGCHEHATCKMTGPGKHKCECKSHYVGDGLNCEPEQLP |
| NOV9v | QKGYKGDGHSCTEIDPCADGLNGGCHEHATCKMTGPGKHKCECKSRYVGDGLNCEPEQLP |
| NOV9w | QKGYKGDGHSCTEIDPCADGLNGGCHEHATCKMTGPGKHKCECKSHYVGDGLNCEPEQLP |
| NOV9x | QKGYKGDGHSCTEIDPCADGLNGGCHEHATCKMTGPGKHKCECKSHYVGDGLNCEPEQLP |
| NOV9y | QKGYKGDGHSCTEIDPCADGLNGGCHEHATCKMTGPGKHKCECKSHYVGDGLNCEPEQLP |
| NOV9a | IDRCLQDNGQCHADAKCVDLHFQDTTVGVFHLRSPLGQYKLTFDKAREACANEAATMATY |
| NOV9b | IDRCLQDNGQCHADAKCVDLHFQDTTVGVFHLRSPLGQYKLTFDKAREACANEAATMATY |
| NOV9c | IDRCLQDNGQCHADAKCADLHFQDTTVGVFHLRSPLGQYKLTFDKAREACANEAATMATY |
| NOV9d | IDRCLQDNGQCHADAKCVDLHFQDTTVGVFHLRSPLGQYKLTFDKAREACANEAATMATY |
| NOV9e | IDRCLQDNGQCHADAKCVDLHFQDTTVGVFHLRSPLGQYKLTFDKAREACANEAATMATY |
| NOV9f | IDRCLQDNGQCHADAKCVDLHFQDTTVGVFHLRSPLGQYKLTFDKAREACANEAATMATY |
| NOV9g | IDRCLQDNGQCHADAKCVDLHFQDTTVGVFHLRSPLGQYKLTFDKAREACANEAATMATY |
| NOV9h | ------------------------------------------------------------ |
| NOV9i | ------------------------------------------------------------ |
| NOV9j | IDRCLQDNGQCHADAKCVDLHFQDTTVGVFHLRSPLGQYKLTFDKAREACANEAATMATY |
| NOV9k | IDRCLQDNGQCHADAKCVDLHFQDTTVGVFHLRSPLGQYKLTFDKAREACANEAATMATY |
| NOV9l | IDRCLQDNGQCHADAKCVDLHFQDTTVGVFHLRSPLGQYKLTFDKAREACANEAATMATY |
| NOV9m | IDRCLQDNGQCHADAKCVDLHFQDTTVGVFHLRSPLGQYKLTFDKAREACANEAATMATY |
| NOV9n | IDRCLQDNGQCHADAKCVDLHFQDTTVGVFHLRSPLGQYKLTFDRAREACANEAATMATY |
| NOV9o | IDRCLQDNGQCHADAKCVDLHFQDTTVGVFHLRSPLGQYKLTFDKAREACANEAATMATY |
| NOV9p | IDRCLQDNGQCHADAKCVDLHFQDTTVGVFHLRSPLGQYKLTFDKAREACANEAATMATY |
| NOV9q | IDRCLQDNGQCHADAKCVDLHFQDTTVGVFHLRSPLGQYKLTFDKAREACANEAATMATY |
| NOV9r | IDRCLQDNGQCHADAKCVDLHFQDTTVGVFHLRSPLGQYKLTFDKAREACANEAATMATY |
| NOV9s | IDRCLQDNGQCHADAKCVDLHFQDTTVGVFHLRSPLGQYKLTFDKAREACANEAATMATY |
| NOV9t | IDRCLQDNGQCHADAKCVDLHFQDTTVGVFHLRSPLGQYKLTFDKAREACANEAATMATY |
| NOV9u | IDRCLQDNGQCHADAKCVDLHFQDTTVGVFHLRSPLGQYKLTFDKAREACANEAATTATY |
| NOV9v | IDRCLQDNGQCHADAKCVDLHFQDTTVGVFHLRSPLGQYKLTFDKAREACANEAATMATY |
| NOV9w | IDRCLQDNGQCHADAKCVDLHFQDTTVGVFHLRSPLGQYKLTFDKAREACANEAATMATY |
| NOV9x | IDRCLQDNGQCHADAKCVDLHFQDTTVGVFHLRSPLGQYKLTFDKAREACANEAATMATY |
| NOV9y | IDRCLQDNGQCHADAKCVDLHFQDTTVGVFHLRSPLGQYKLTFDKAREACANEAATMATY |
| NOV9a | NQLSYAQKAKYHLCSAGWLETGRVAYPTAFASQNCGSGVVGIVDYGPRPNKSEMWDVFCY |
| NOV9b | NQLSYAQKAKYHLCSAGWLETGRVAYPTAFASQNCGSGVVGIVDYGPRPNKSEMWDVFCY |
| NOV9c | NQLSYAQKAKYHLCSAGWLETGRVAYPTAFASQNCGSGVVGIVDYGPRPNKSEMWDVFCY |
| NOV9d | NQLSYAQKREEK------------------------------------------------ |
| NOV9e | NQLSYAQKTWYSFTKE-------------------------------------------- |
| NOV9f | NQLSYAQKTWYSFTKE-------------------------------------------- |
| NOV9g | NQLSYAQKTWYSFTKE-------------------------------------------- |
| NOV9h | ------------------------------------------------------------ |
| NOV9i | ------------------------------------------------------------ |
| NOV9j | NQLSYAQKTWYSFTKE-------------------------------------------- |
| NOV9k | NQLSYAQKAKYHLCSAGWLETGRVAYPTAFASQNCGSGVVGIVDYGPRPNKSEMWDVFCY |
| NOV9l | NQLSYAQKAKYHLCSAGWLETGRVAYPTAFASQNCGSGVVGIVDYGPRPNKSEMWDVFCY |
| NOV9m | NQLSYAQKAKYHLCSAGWLETGRVAYPTAFASQNCGSGVVGIVDYGPRPNKSEMWDVFCY |
| NOV9n | NQLSYAQKAKYHLCSAGWLETGRVAYPTAFASQNCGSGVVGIVDYGPRPNKSEMWDVFCY |
| NOV9o | NQLSYAQKAKYHLCSAGWLETGRVAYPTAFASQNCGSGVVGIVDYGPRPNKSEMWDVFCY |
| NOV9p | NQLSYAQKAKYHLCSAGWLETGRVAYPTAFASQNCGSGVVGIVDYGPRPNKSEMWDVFCY |
| NOV9q | NQLSYAQKAKYHLCSAGWLETGRVAYPTAFASQNCGSGVVGIVDYGPRPNKSEMWDVFCY |
| NOV9r | NQLSYAQKAKYHLCSAGWLETGRVAYPTAFASQNCGSGVVGIVDYGPRPNKSEMWDVFCY |
| NOV9s | NQLSYAQKAKYHLCSAGWLETGRVAYPTAFASQNCGSSVVGIVDYGPRPNKSEMWDVFCY |
| NOV9t | NQLSYAQKAKYHLCSAGWLETGRVAYPTAFASQNCGSGVVGIVDYGPRPNKSEMWDVFCY |
| NOV9u | NQLSYAQKAKYHLCSAGWLETGRVAYPTAFASQNCGSGVVGIVDYGPRPNKSEMWDVFCY |
| NOV9v | NQLSYAQKAKYHLCSAGWLETGRVAYPTAFASQNCGSGVVGIVDYGPRPNKSEMWDVFCY |
| NOV9w | NQLSYAQKAKYHLCSAGWLETGRVAYPTAFASQNCGSGVVGIVDYGPRPNKSEMWDVFCY |
| NOV9x | NQLSYAQKAKYHLCSAGWLETGRVAYPTAFASQNCGSGVVGIVDYGPRPNKSEMWDVFCY |
| NOV9y | NQLSYAQKAKYHLCSAGWLETGRVAYPTAFASQNCGSGVVGIVDYGPRPNKSEMWDVFCY |
| NOV9a | RMKDVNCTCKVGYVGDGFSCSGNLLQVLMSFPSLTNFLTEVLAYSNSSARGRAFLEHLTD |
| NOV9b | RP---------------------------------------------------------- |
| NOV9c | RMKGS----------------AGLFQQLSSRPCISRTPD--------------------- |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | RMKGS----------------AGLFQQLSSRPCISRTPD--------------------- |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | RMKDVNCTCKVGYVGDGFSCSGNLLQVLMSFPSLTNFLTEVLAYSNSSARGRAFLEHLTD |
| NOV9x | RMKDVNCTCKVGYVGDGFSCSGNLLQVLNSFPSLTNFLTEVLAYSNSSARGRAFLEHLTD |
| NOV9y | RMKDVNCTCKVGYVGDGFSCSGNLLQVLMSFPSLTNFLTEVLAYSNSSARGRAFLEHLTD |
| NOV9a | LSIRGTLFVPQNSGLGENETLSGRDIEHHLANVSMFFYNDLVNGTTLQTRVGSKLLITAS |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | ------------------------------------------------------------ |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | LSIRGTLFVPQNSGLGENETLSGRDIEHHLANVSMFFYNDLVNGTTLQTRVGSKLLITAS |
| NOV9x | LSIRGTLFVPQNSGLGENETLSGRDIEHHLANVSMFFYNDLVNGTTLQTRLGSKLLITAS |
| NOV9y | LSIRGTLFVPQNSGLGENETLSGRDIEHHLANVSMFFYNDLVNGTTLQTRLGSKLLITAS |
| NOV9a | QDPLQPTETRFVDGRAILQWDIFASNGIIHVISRPLKAPPAPVTLTHTGLGAGIFFAIIL |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | ------------------------------------------------------------ |
| NOV9l | ------------------------------------------------------------ |
| NOV9m | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | QDPLQPTETRFVDGRAILQWDIFASNGIIHVISRPLKAPPAPVTLTHTGLGAGIFFAIIL |
| NOV9x | QDPLQPTETRFVDGPAILQWDIFASNGIIHVISRPLKAPPAPVTLTHTGLGAGIFFAIIL |
| NOV9y | QDPLQPTETRFVDGRAILQWDIFASNGIIHVISRPLKAPPAPVTLTHTGLGAGIFFAIIL |
| NOV9a | VTGAVALAAYSYFRINRRTIGFQHFESEEDINVAALGKQQPENISNPLYESTTSAPPEPS |
| NOV9b | ------------------------------------------------------------ |
| NOV9c | ------------------------------------------------------------ |
| NOV9d | ------------------------------------------------------------ |
| NOV9e | ------------------------------------------------------------ |
| NOV9f | ------------------------------------------------------------ |
| NOV9g | ------------------------------------------------------------ |
| NOV9h | ------------------------------------------------------------ |
| NOV9i | ------------------------------------------------------------ |
| NOV9j | ------------------------------------------------------------ |
| NOV9k | ------------------------------------------------------------ |
| NOV9l | ------------------------------------------------------------ |
| NOV9m | ------------------------------------------------------------ |
| NOV9n | ------------------------------------------------------------ |
| NOV9o | ------------------------------------------------------------ |
| NOV9p | ------------------------------------------------------------ |
| NOV9q | ------------------------------------------------------------ |
| NOV9r | ------------------------------------------------------------ |
| NOV9s | ------------------------------------------------------------ |
| NOV9t | ------------------------------------------------------------ |
| NOV9u | ------------------------------------------------------------ |
| NOV9v | ------------------------------------------------------------ |
| NOV9w | VTGAVALAAYSYFRINRRTIGFQHFESEEDINVAALGKQQPENISNPLYESTTSAPPEPS |
| NOV9x | VTGAVALAAYSYFRINRRTIGFQHFESEEDINVAALGKQQPENISNPLYESTTSAPPEPS |
| NOV9y | VTGAVALAAYSYFRINRRTIGFQHFESEEDINVAALGKQQPENISNPLYESTTSAPPEPS |
| NOV9a | YDPFTDSEERQLEGNDPLRTL |
| NOV9b | --------------------- |
| NOV9c | --------------------- |
| NOV9d | --------------------- |
| NOV9e | --------------------- |
| NOV9f | --------------------- |
| NOV9g | --------------------- |
| NOV9h | --------------------- |
| NOV9i | --------------------- |
| NOV9j | --------------------- |
| NOV9k | --------------------- |
| NOV9l | --------------------- |
| NOV9m | --------------------- |
| NOV9n | --------------------- |
| NOV9o | --------------------- |
| NOV9p | --------------------- |
| NOV9q | --------------------- |
| NOV9r | --------------------- |
| NOV9s | --------------------- |
| NOV9t | --------------------- |
| NOV9u | --------------------- |
| NOV9v | --------------------- |
| NOV9w | YDPFTDSEERQLEGNDPLRTL |
| NOV9x | YDPFTDSEERQLEGNDPLRTL |
| NOV9y | YDPFTDSEERQLEGNDPLRTL |
| NOV9a | (SEQ ID NO: 208) |
| NOV9b | (SEQ ID NO: 210) |
| NOV9c | (SEQ ID NO: 212) |
| NOV9d | (SEQ ID NO: 214) |
| NOV9e | (SEQ ID NO: 216) |
| NOV9f | (SEQ ID NO: 218) |
| NOV9g | (SEQ ID NO: 220) |
| NOV9h | (SEQ ID NO: 222) |
| NOV9i | (SEQ ID NO: 224) |
| NOV9j | (SEQ ID NO: 226) |
| NOV9k | (SEQ ID NO: 228) |
| NOV9l | (SEQ ID NO: 230) |
| NOV9m | (SEQ ID NO: 232) |
| NOV9n | (SEQ ID NO: 234) |
| NOV9o | (SEQ ID NO: 236) |
| NOV9p | (SEQ ID NO: 238) |
| NOV9q | (SEQ ID NO: 240) |
| NOV9r | (SEQ ID NO: 242) |
| NOV9s | (SEQ ID NO: 244) |
| NOV9t | (SEQ ID NO: 246) |
| NOV9u | (SEQ ID NO: 248) |
| NOV9v | (SEQ ID NO: 250) |
| NOV9w | (SEQ ID NO: 252) |
| NOV9x | (SEQ ID NO: 254) |
| NOV9y | (SEQ ID NO: 256) |

Further analysis of the NOV9a protein yielded the following properties shown in Table 9C.

TABLE 9C
Protein Sequence Properties NOV9a

| | |
|---|---|
| SignalP analysis: | Cleavage site between residues 32 and 33 |
| PSORT II analysis: | |

| PSG: | a new signal peptide prediction method | |
|---|---|---|
| | N-region: | length 6; pos. chg 1; neg. chg 0 |
| | H-region: | length 11; peak value 3.91 |
| | PSG score: | −0.49 |

| | |
|---|---|
| GvH: | von Heijne's method for signal seq. recognition |
| | GvH score (threshold: −2.1): −5.51 |
| | possible cleavage site: between 28 and 29 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 2 | | |
| | Number of TMS(s) for threshold 0.5: 1 | | |
| | INTEGRAL | Likelihood = | Transmembrane 808-824 |
| | | −8.33 | |

| | |
|---|---|
| PERIPHERAL | Likelihood = 2.97 (at 663) |

| | |
|---|---|
| | ALOM score: −8.33 (number of TMSs: 1) |
| MTOP: | Prediction of membrane topology (Hartmann et al.) |
| | Center position for calculation: 815 |
| | Charge difference: −1.0 C(1.5)-N(2.5) |
| | N >= C: N- terminal side will be inside |

>>> Single TMS is located near the C-terminus
>>> membrane topology: type Nt (cytoplasmic tail 1 to 807)

| MITDISC: | discrimination of mitochondrial targeting seq | | | |
|---|---|---|---|---|
| | R content: | 1 | Hyd Moment(75): | 5.98 |
| | Hyd Moment(95): | 9.18 | G content: | 0 |
| | D/E content: | 1 | S/T content: | 0 |

| | |
|---|---|
| | Score: −3.74 |
| Gavel: | prediction of cleavage sites for mitochondrial preseq |
| | R-2 motif at 16 LRY\|HV |

NUCDISC: discrimination of nuclear localization signals

| | |
|---|---|
| | pat4: none |
| | pat7: none |
| | bipartite: none |
| | content of basic residues: 8.7% |
| | NLS Score: −0.47 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: found

LL at 15
LL at 16
LL at 69
LL at 75
LL at 111
LL at 213
LL at 226
LL at 660

checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic /Nuclear discrimination |
| | Prediction: nuclear |
| | Reliability: 55.5 |
| COIL: | Lupas's algorithm to detect coiled-coil regions |
| | total: 0 residues |

Final Results (k = 9/23):

```
30.4%: nuclear
21.7%: cytoplasmic
13.0%: mitochondrial
13.0%: Golgi
  8.7%: vesicles of secretory system
  8.7%: endoplasmic reticulum
  4.3%: peroxisomal
```

>> prediction for CG50736-09 is nuc (k = 23)

A search of the NOV9a protein against the Geneseq database, a proprietary database that contains sequences published in patents and patent publication, yielded several homologous proteins shown in Table 9D.

TABLE 9D
Geneseq Results for NOV9a

| | | NOV9a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABJ10588 | Human novel protein NOV1c SEQ ID | 1 . . . 897 | 897/897 (100%) | 0.0 |
| | NO: 211 - Homo sapiens, 897 aa. | 1 . . . 897 | 897/897 (100%) | |
| | [WO200259315-A2, 01-AUG-2002] | | | |
| ABG72514 | Human 190 kDa Hyaluronan receptor | 1 . . . 897 | 896/897 (99%) | 0.0 |
| | for endocytosis (HARE) #2 - Homo | 757 . . . 1653 | 897/897 (99%) | |
| | sapiens, 1653 aa. [WO200286093-A2, | | | |
| | 31-OCT-2002] | | | |
| ABG72499 | Human 190 kDa Hyaluronan receptor | 1 . . . 897 | 896/897 (99%) | 0.0 |
| | for endocytosis (HARE) #1 - Homo | 520 . . . 1416 | 897/897 (99%) | |
| | sapiens, 1416 aa. [WO200286093-A2, | | | |
| | 31-OCT-2002] | | | |
| AAM47684 | Human Hyaluronic Acid Receptor for | 1 . . . 897 | 896/897 (99%) | 0.0 |
| | Endocytosis, HARE - Homo sapiens, | 498 . . . 1394 | 897/897 (99%) | |
| | 1394 aa. [WO200181544-A2, 01-NOV- | | | |
| | 2001] | | | |
| ABG72498 | Rat 175 kDa Hyaluronan receptor for | 1 . . . 886 | 674/887 (75%) | 0.0 |
| | endocytosis (HARE) - Rattus sp, 1431 | 519 . . . 1404 | 758/887 (84%) | |
| | aa. [WO200286093-A2, 31-OCT-2002] | | | |

In a BLAST search of public sequence databases, the NOV9a protein was found to have homology to the proteins shown in the BLASTP data in Table 9E.

TABLE 9E
Public BLASTP Results for NOV9a

| | | NOV9a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9NRY3 | CD44-like precursor FELL - Homo | 1 . . . 897 | 897/897 (100%) | 0.0 |
| | sapiens (Human), 897 aa. | 1 . . . 897 | 897/897 (100%) | |
| AAO39681 | Hyaluronan receptor for | 1 . . . 897 | 896/897 (99%) | 0.0 |
| | endocytosis - Homo sapiens | 520 . . . 1416 | 897/897 (99%) | |
| | (Human), 1416 aa (fragment). | | | |
| Q8IUG9 | FELE-2 - Homo sapiens (Human), | 1 . . . 897 | 896/897 (99%) | 0.0 |
| | 2551 aa. | 1655 . . . 2551 | 897/897 (99%) | |
| Q8WWQ8 | Stabilin-2 - Homo sapiens (Human), | 1 . . . 897 | 896/897 (99%) | 0.0 |
| | 2551 aa. | 1655 . . . 2551 | 897/897 (99%) | |
| Q9H7H7 | FLJ00112 protein - Homo sapiens | 1 . . . 897 | 896/897 (99%) | 0.0 |
| | (Human), 1192 aa (fragment). | 296 . . . 1192 | 897/897 (99%) | |

PFam analysis predicts that the NOV9a protein contains the domains shown in the Table 9F.

TABLE 9F
Domain Analysis of NOV9a

| | | Identities/ | |
|---|---|---|---|
| Pfam | NOV9a | Similaritiesfor | Expect |
| 0 Domain | Match Region | the Matched Region | Value |
| Fasciclin | 86 . . . 229 | 38/152 (25%) | 1.5e−08 |
| | | 100/152 (66%) | |
| EGF | 355 . . . 387 | 13/47 (28%) | 0.24 |
| | | 22/47 (47%) | |
| EGF | 397 . . . 426 | 13/47 (28%) | 0.054 |
| | | 23/47 (49%) | |
| EGF | 432 . . . 467 | 10/47 (21%) | 0.066 |
| | | 23/47 (49%) | |
| EGF | 473 . . . 510 | 13/47 (28%) | 0.036 |
| | | 31/47 (66%) | |
| Xlink | 543 . . . 636 | 40/104 (38%) | 1.1e−26 |
| | | 69/104 (66%) | |

### Example 10

The NOV10 clone was analyzed, and the nucleotide and encoded polypeptide sequences are shown in Table 10A.

TABLE 10A
NOV10 Sequence Analysis

| | | |
|---|---|---|
| NOV10a, CG50925-01 | SEQ ID NO: 257 | 7892 bp |
| DNA Sequence | ORF Start: ATG at 1 | ORF Stop: TAA at 4627 |

ATGCGGGGGGCGCCCGCGCGCCTGCTGCTGCCGCTGCTGCCGTGGCTCCTGCTGCTCCTGGCGCCCGA
GGCTCGGGGCGCGCCCGGCTGCCCGCTATCCATCCGCAGCTGCAAGTGCTCGGGGGAGCGGCCCAAGG
GGCTGAGCGGCGGCGTCCCTGGCCCGGCTCGGCGGAGGGTGGTGTGCAGCGGCGGGGACCTCCCGGAG
CCTCCCGAGCCCGGCCTTCTGCCTAACGGCACCGTTACCCTTGGCAACCGTTTCTCCACTGCCCCTTG
CCCGCAGTCGGGGCTGGCCCAGTCTTTTCGATTGTGTCCAGAACAATACAGTAACGCCTCTGGTTGGG
TGAGTTCAGAGGCTGCCCGCTTCCTGACAGGACACAACTGGTCAGCCTCTGACGCTGGCCGCCCGGCC
TGCATTTTCCCCGGCCCGGTCTCCTGGAGACAGGTACCAGCTTACAACGCCGGACAGGATCAGGGTAG
CAAATATAGCTTCTATGGAGGAAAGGTCAAAGATGAGTATCTGCGTAGGTCCCCGATCTGTCGCTCTG
CTGCCGCCCTGCACACTGACACCAGTGCCCGCAGCTGGTCTGAATTTAGGCTCTTGAGCAATAACAAG
ATCACGGGGCTCCGCAATGGCTCCTTCCTGGGACTGTCACTGCTGGAGAAGCTTCTCATGAGAGCACA
TTTTTCTGATGAAGATGAAAAGACGGCCTGTCCTTCTCCAGCACCAAGGCTCTGGAGGCTGCATAGCA
AGTGCACCCTGAGCATGGCAGGCAATCAGAAGAACCAGGGCGGCAGCAGCTCACAAGAGGCAGCATCT
GGCTTGATGCCGAGTCGGCGGCTCCAGCGGGGAGAGGTTGAGGGCCTCTGCACTTGCCTCTGCACGGA
CCTGAGGAACAACATCATCAGCACAGTGCAGCCGGGCGCCTTCCTGGGCCTGGGGGAGCTGAAGCGTT
TAGATCTCTCCAACAACCGGATTGGCTGTCTCACCTCCGAGACCTTCCAGGGCCTCCCCAGGCTTCTC
CGACTAAACATATCTGGAAACATCTTCTCCAGTCTGCAACCTGGGGTCTTTGATGAGCTGCCAGCCCT
TAAGGTTGTGGACTTGGGCACCGAGTTCCTGACCTGTGACTGCCACCTGCGCTGGCTGCTGCCCTGGG
CCCAGAATCGCTCCCTGCAGCTGTCGGAACACACGCTCTGTGCTTACCCCAGTGCCCTGCATGCTCAT
GCCCTGGGCAGCCTCCAGGAGGCCCAGCTCTGCTGCGAGGGGGCCCTGGAGCTGCACACACACCACCT
CATCCCGTCCCTACGCCAAGTGGTGTTCCAGGGGGATCGGCTGCCCTTCCAGTGCTCTGCCAGCTACC
TGGGCAACGACACCCGCATCCGCTGGTACCACAACCGAGCCCCTGTGGAGGGTGATGAGCAGGCGGGC
ATCCTCCTGGCCGAGAGCCTCATCCACGACTGCACCTTCATCACCAGTGAGCTGACGCTGTCTCACAT
CGGCGTGTGGGCCTCAGGCGAGTGGGAGTGCACCGTGTCCATGGCCCAAGGCAACGCCAGCAAGAAGG
TGGAGATCGTGGTGCTGGAGACCTCTGCCTCCTACTGCCCCGCCGAGCGTGTTGCCAACAACCGCGGG
GACTTCAGGTGGCCCCGAACTCTGGCTGGCATCACAGCCTACCAGTCCTGCCTGCAGTATCCCTTCAC
CTCAGTGCCCCTGGGCGGGGGTGCCCCGGGCACCCGAGCCTCCCGCCGGTGTGACCGTGCCGGCCGCT
GGGAGCCAGGGGACTACTCCCACTGTCTCTACACCAACGACATCACCAGGGTGCTGTACACCTTCGTG
CTGATGCCCATCAATGCCTCCAATGCGCTGACCCTGGCTCACCAGCTGCGCGTGTACACAGCCGAGGC
CGCTAGCTTTTCAGACATGATGGATGTAGTCTATGTGGCTCAGATGATCCAGAAATTTTTGGGTTATG
TCGACCAGATCAAAGAGCTGGTAGAGGTGATGGTGGACATGCCCAGCAACTTGATGCTGGTGGACGAG
CACCTGCTGTGGCTGGCCCAGCGCGAGGACAAGGCCTGCAGCCGCATCGTGGGTGCCATAGAGCGCAT
TGGGGGGGCCGCCCTCAGCCCCCATGCCCAGCACATCTCAGTGAATGCGAGGAACGTGGCATTGGAGG
CCTACCTCATCAAGCCGCACAGCTACGTGGGCCTGACCTGCACAGCCTTCCAGAGGAGGGAGGGAGGG
GTGCCGGGCACACGGCCAGGAAGCCCTGGCCAGAACCCCCCACCTGAGCCCGAGCCCCCAGCTGACCA
GCAGCTCCGCTTCCGCTGCACCACCGGGAGGCCCAATGTTTCTCTGTCGTCCTTCCACATCAAGAACA
GCGTGGCCCTGGCCTCCATCCAGCTGCCCCCGAGTCTATTCTCATCCCTTCCGGCTGCCCTGGCTCCC
CCGGTGCCCCCAGACTGCACCCTGCAACTGCTCGTCTTCCGAAATGGCCGCCTCTTCCACAGCCACAG
CAACACCTCCCGCCCTGGAGCTGCTGGGCCTGGCAAGAGGCGTGGCGTGGCCACCCCCGTCATCTTCG
CAGGAACCAGTGGCTGTGGCGTGGGAAACCTGACAGAGCCAGTGGCCGTTTCGCTGCGGCACTGGGCT
GAGGGAGCCGAACCTGTGGCCGCTTGGTGGAGCCAGGAGGGGCCCGGGGAGGCTGGGGGCTGGACCTC
GGAGGGCTGCCAGCTCCGCTCCAGCCAGCCCAATGTCAGCGCCCTGCACTGCCAGCACTTGGGCAATG
TGGCCGTGCTCATGGAGCTGAGCGCCTTTCCCAGGGAGGTGGGGGGCGCCGGGGCAGGGCTGCACCCC
GTGGTATACCCCTGCACGGCCTTGCTGCTGCTCTGCCTCTTCGCCACCATCATCACCTACATCCTCAA
CCACAGCTCCATCCGTGTGTCCCGGAAAGGCTGGCACATGCTGCTGAACTTGTGCTTCCACATAGCCA
TGACCTCTGCTGTCTTTGCGGGGGGCATCACACTCACCAACTACCAGATGGTCTGCCAGGCGGTGGGC
ATCACCCTGCACTACTCCTCCCTATCCACGCTGCTCTGGATGGGCGTGAAGGCGCGAGTGCTCCATAA
GGAGCTCACCTGGAGGGCACCCCCTCCGCAAGAAGGGGACCCCGCTCTGCCTACTCCCAGTCCTATGC
TCCGGTACATACTTTCAATTCCAGCTTTGCAATTGGGGAGGGACTCCAACGCAGGCGTAGGAAACCTC
CCAAGGTTCTATTTGATCGCTGGAGGGATTCCACTCATTATCTGTGGCATCACAGCTGCAGTCAACAT
CCACAACTACCGGGACCACAGCCCCTACTGCTGGCTGGTGTGGCGTCCAAGCCTTGGCGCCTTCTACA
TCCCTGTGGCTTTGATTCTGCTCATCACCTGGATCTATTTCCTGTGCGCCGGGCTACGCTTACGGGGT
CCTCTGGCACAGAACCCCAAGGCGGGCAACAGCAGGGCCTCCCTGGAGGCAGGGGAGGAGCTGAGGGG
TTCCACCAGGCTCAGGGGCAGCGGCCCCCTCCTGAGTGACTCAGGTTCCCTTCTTGCTACTGGGAGCG
CGCGAGTGGGGACGCCCGGGCCCCCGGAGGATGGTGACAGCCTCTATTCTCCGGGAGTCCAGCTAGGG
GCGCTGGTGACCACGCACTTCCTGTACTTGGCCATGTGGGCCTGCGGGGCTCTGGCAGTGTCCCAGCG
CTGGCTGCCCCGGGTGGTGTGCAGCTGCTTGTACGGGGTGGCAGCCTCCGCCCTGGGCCTCTTCGTCT
TCACTCACCACTGTGCCAGGCGGAGGGACGTGAGAGCCTCGTGGCGCGCCTGCTGCCCCCCTGCCTCT
CCCGCGGCCCCCCATGCCCCGCCCCGGGCCCTGCCCGCCGCCGCAGAGGACGGTTCCCCGGTGTTCGG
GGAGGGGCCCCCCTCCCTCAAGTCCTCCCCAAGCGGCAGCAGCGGCCATCCGCTGGCTCTGGGCCCCT
GCAAGCTCACCAACCTGCAGCTGGCCCAGAGTCAGGTGTGCGAGGCGGGGGCGGCGGCCGGCGGGGAA
GGAGAGCCGGAGCCGGCGGGCACCCGGGGAAACCTCGCCCACCGCCACCCCAACAACGTGCACCACGG
GCGTCGGGCGCACAAGAGCCGGGCCAAGGGACACCGCGCGGGGGAGGCCTGCGGCAAGAACCGGCTCA
AGGCCCTGCGCGGGGGCGCGGCGGGGGCGCTGGAACTGCTGTCCAGCGAGAGCGGTAGTCTGCACAAC
AGCCCCACCGACAGCTACCTGGGCAGCAGCCGCAACAGCCCGGGCGCCGGCCTGCAGCTGGAAGGCGA
GCCCATGCTCACGCCGTCCGAGGGCAGCGACACCAGCGCCGCGCCGCTTTCTGAGGCGGGCCGGGCAG
GCCAGCGCCGCAGCGCCAGCCGCGACAGTCTCAAGGGCGGCGGCGCGCTGGAGAAGGAGAGCCATCGC
CGCTCGTACCCGCTCAACGCCGCCAGCCTAAACGGCGCCCCCAAGGGGGGCAAGTACGACGACGTCAC
CCTGATGGGCGCGGAGGTAGCCAGCGGCGGCTGCATGAAGACCGGACTCTGGAAGAGCGAAACTACCG
TCTAAGGTGGGGCGGGCGACGCGGTAGACGGGTTGGCCACGCGGCTCGTTCCCCCGCTCCTCGGGGCC
CTCCAAGGTGTCTCCGTAGTCAGCAGGTTGGAGGCAGAGGAGCCGATGGCTGGAGGAAGCCCACAGGC
GGATGTTCCCCACTTGCCTAGAGGGCATCCCTCTGGGGTAGCGACAGACAATCCCAGAAACACGCATA
ATACATTTCCGTCCAGCCCGGGGCAGTCTGACTGTCGGTGCCCTCCCAGGAACGGGGAAGGCCTCCGT
CTGTGTGAAAGGGCACAGCACATCCCAGGTGCACCCTCCCCAAGTACTCCCACCCCGCCTACTGTCCA
TGCGGCCTCACTGGGGGCCATCAGCCTCACCAGCAAAGCAGAGATGAGAGCGTGGGAACTGTGTTCTT
TCCTCCCTGCCCTCTACTGATTTCAGCCCAGCCCCTGCCTAGATCCTAGGTCCCTTTTCCTCCCGAGT
TTGGCTGGCACGAGAGCTAGCCCAGCACATGAAGCAGGTGATGTTAAGTCACAAGGTGCTGCTTTTCA
GATCCACTATGCAAGAGGGGAGGGTGGGGCCACGTGAAAGGCAGCTCTAGACATCAACCAGTCCTGGG
GGAGGGGAGTGGGAACCGGGCACAACTAGGAACAATGCCACCATTCCCACAGGAGTGGTACTTAAACC
AGACAGCAGGGTTCAGAGGTGGCACACCGGGACAAAGCTGAGGCCCTGCACCTCAACAGCTGACTGCC
AGGTGCCTGTGGGTGAACTGAGGGGAGTAGAGGGAGAGGGCAGGTGGAACTGGGGCAGAATCTAGTCA
TGCCCTAAAGCTAGTCCTGTAAACAATGGTGCCCCAGAAAGCTGCAGGTGGTGTTTGGAGAAGCAGTT
ACTTTTCAGTTACAAGACCCATCTCCCTAGTCTCAGCCTTACAACACCACGGGACTAAGGAAGAGCAC
TTCCTTGCCTCCGTAAGGCCAGAGGAAGAACCATCCCAATCATTTGATCTCCAGCTCCACAGTAGAGA
GAAACCTACAAAATGTCAAACCAGCTTCCCGACTCCCAGGAGCTCAAGCCAAGCCCAGAGGCAGTGGC
TGGGGTCCCTGCAGGTCATGAGGGGCCTATGCCTTTACTCCTTTTAAACACCAGCACCCGTCTTTTCC
CCAACCTAAAACCAACCACCAGCATTTCACTACAGGACCAAATGGAAACCGAGGGAACCCTGGGTCTT
GGGAAGAACAACAGGAAACCAAGGTCTGACCTAGGGTTCCCTCCCAGTCTTCACATCACTCTGGCCTC
ATCACCAAGGTGACAGAGGACACAGGGGAGGGGGAAAACCCACACACACTCCTTGGAATGGGTCCTGT
TATTTATGCTTGCTGCACAGACATATTAGAAGAAAAAAAAAAAGCTTTGTATTATTCTTCCACATATG
CTGGCTGCTGTTTACACACCCTGCCAATGCCTTAGCACTGGAGAGCTTTTTGCAATATGCTGGGGAAA
GGGGAGGGAGGGAATGAAAGTGCCAAAGAAAACATGTTTTTAAGAACTCGGGTTTTATACAATAGAAT
GTTTTCTAGCAGATGCCTCTTGTTTTAATATATTAAAATTTTGCAAAGCCCTTTGAGCTACTGCCTTA
GTCTACCCACTGTCCTTTTGTTATGAGGTAGAGGATCTCATGACACCATACACACAAACCCATCATTG
CCTGTGAATGCACGTAGGGCCAGAATTCCCCAGTTCCCGCTCCTCTGAGGGTTGATACTGCTGGGAAT
GCCAACCACTCCACAAGCAGAGGGAAGCCCCCTCAGGCCTGCAGGAGGAGCCGCAGCAGTGTGTCCAA
TTCAAACCAGCAGCAAAGAGCCTGACATTTTCCCATCCATCTATGAGGAAAGCCATCTCACAGAACAT
GGACATAGGCAACTTGCTCTCCCACACCAAGGGATGGGAATCTCTCCTACCTATAGTCATCCCTGCAC
TCCTGACTTTACTCCAGGACCCAGGGTCCAACTAATGGCAGAGCCCCTCTTGGTTCCTTCAAACAAGA
AAAGCAATACCTACGGACTGGTGTACACTTCCATCCTTGGTTATAACAGGAATGTTATCAAGCTGTCA
GAACAGGATGAAGTGCTCCCAGTGGATATCCATCAGGGAGGGTTAGGGACACTCGTGGCAGCCTGTCT
AGCAGCCTGGGCTCTCTGAAAGTCCCTAACTTCCTGAGGGGTACGCAAATACTGTTCTATTTCACTAT
CAGAAATGTTCTCATCTCCAGTGACAGTGGAGACAGGGGGTACAGGGCAGATCCGCTTCGGGGACTTC
AACATGCAGGGTGGCAAGAGAAGGGCAGGACTGGCCGGCCGCTTCCCCTGGGGTAAACCTAAGGAATT
ATTTCCCACCTCCCCTTCTCCTTGCCCCTGTCCCCACCCCGGTGGCTCCTTCTCTCGGGTCTCCACTT
CTGCTGTCCCATCCCGAAAGGCAGAGCGGACCAGTGACTGGCGGTGCTGGAGAAGGTCACCGATGTGC
TTCACCACAGACCGTTTGTCAAGTCTCAGAACTCGTAACCAGGCCAGCTGCTCAGCCATCCGCAGCAG
CACAGCCAGCAGCTCCTGCAGGCGGGAGGACGCCGGGTAGGGCAGGTCCACATTTGCCAATTTACAAA
ATCGGGCAAGGGAACATGAAAGCCGATCTGCAGGCTGCAGCGACTGCCAAGCCAGGAAAGTCGCAGCA
GTGATGACGGGCAAGGGATGCCTCCCGGTCACCAGCCACGTCTCATTTGCCAGCTCCACCAACTGCAT
TGTTCGAGACAGCATCTTCTCTTTGTCTTCCACGTATTTGGCTGGCACAGAAGGTGAAGCTTGGAACA
GTTTGAAGCTGAAATAACCAAAATGAGGGTTGGATCTTAATGATATAGGGGCTGCTCTCCCACAGTGA
GGAAAGACAGCCCACTCAAGATGGGGAAGCTATTCTGCCCTCAGGAATACTCAAGCTCACTGGGCAGC
AAGTTAATAAAGGTAGTGAGAGAAAACAGGGCGTCTTCCGCTTGTTAGGGGAAGGTGGAGGGATGGAG
GAGAGCACGAACATTTATTGGGCGCCTCCCAATCACCATTATTCTGAGTGCTTTACAACGTTCTCATT
TAATCTACGTGCACGTGCACCATCTTATGTGCATGTATAGTTAAAAAACTTTCCCATAGTCATCCAGC
CAGGCAGTAACCAAGCTTCAAATACAAGGCTATTTGACACCAACAGCCTCTACTTTCAACGTTATTTA
TCAG

| | | | |
|---|---|---|---|
| NOV10a, CG50925-01 | SEQ ID NO: 258 | 1542 aa | MW at 164901.2kD |
| Protein Sequence | | | |

MRGAPARLLLPLLPWLLLLLAPEARGAPGCPLSIRSCKCSGERPKGLSGGVPGPARRRVVCSGGDLPE
PPEPGLLPNGTVTLGNRFSTAPCPQSGLAQSFRLCPEQYSNASGWVSSEAARFLTGHNWSASDAGRPA
CIFPGPVSWRQVPAYNAGQDQGSKYSFYGGKVKDEYLRRSPICRSAAALHTDTSARSWSEFRLLSNNK
ITGLRNGSFLGLSLLEKLLMRAHFSDEDEKTACPSPAPRLWRLHSKCTLSMAGNQKNQGGSSSQEAAS
GLMPSRRLQRGEVEGLCTCLCTDLRNNIISTVQPGAFLGLGELKRLDLSNNRIGCLTSETFQGLPRLL
RLNISGNIFSSLQPGVFDELPALKVVDLGTEFLTCDCHLRWLLPWAQNRSLQLSEHTLCAYPSALHAH
ALGSLQEAQLCCEGALELHTHHLIPSLRQVVFQGDRLPFQCSASYLGNDTRIRWYHNRAPVEGDEQAG
ILLAESLIHDCTFITSELTLSHIGVWASGEWECTVSMAQGNASKKVEIVVLETSASYCPAERVANNRG
DFRWPRTLAGITAYQSCLQYPFTSVPLGGGAPGTRASRRCDRAGRWEPGDYSHCLYTNDITRVLYTFV
LMPINASNALTLAHQLRVYTAEAASFSDNMDVVYVAQMIQKFLGYVDQIKELVEVMVDMPSNLMLVDE
HLLWLAQREDKACSRIVGAIERIGGAALSPHAQHISVNARNVALEAYLIKPHSYVGLTCTAFQRREGG
VPGTRPGSPGQNPPPEPEPPADQQLRFRCTTGRPNVSLSSFHIKNSVALASIQLPPSLFSSLPAALAP
PVPPDCTLQLLVFRNGRLFHSHSNTSRPGAAGPGKRRGVATPVIFAGTSGCGVGNLTEPVAVSLRHWA
EGAEPVAAWWSQEGPGEAGGWTSEGCQLRSSQPNVSALHCQHLGNVAVLMELSAFPREVGGAGAGLHP
VVYPCTALLLLCLFATIITYILNHSSIRVSRKGWHMLLNLCFHIAMTSAVFAGGITLTNYQMVCQAVG
ITLHYSSLSTLLWMGVKARVLHKELTWRAPPPQEGDPALPTPSPMLRYILSIPALQLGRDSNAGVGNL
PRFYLIAGGIPLIICGITAAVNIHNYRDHSPYCWLVWRPSLGAFYIPVALILLITWIYFLCAGLRLRG
PLAQNPKAGNSRASLEAGEELRGSTRLRGSGPLLSDSGSLLATGSARVGTPGPPEDGDSLYSPGVQLG
ALVTTHFLYLAMWACGALAVSQRWLPRVVCSCLYGVAASALGLFVFTHHCARRRDVRASWRACCPPAS
PAAPHAPPRALPAAAEDGSPVFGEGPPSLKSSPSGSSGHPLALGPCKLTNLQLAQSQVCEAGAAAGGE
GEPEPAGTRGNLAHRHPNNVHHGRRAHKSRAKGHRAGEACGKNRLKALRGGAAGALELLSSESGSLHN
SPTDSYLGSSRNSPGAGLQLEGEPMLTPSEGSDTSAAPLSEAGRAGQRRSASRDSLKGGGALEKESHR
RSYPLNAASLNGAPKGGKYDDVTLMGAEVASGGCMKTGLWKSETTV

| | | |
|---|---|---|
| NOV10b, CG50925-08 | SEQ ID NO: 259 | 4103 bp |
| DNA Sequence | ORF Start: ATG at 1 | ORF Stop: TAA at 3991 |

ATGCGGGGGGCGCCCGCGCGCCTGCTGCTGCCGCTGCTGCCGTGGCTCCTGCTGCTCCTGGCGCCCGA
GGCTCGGGGCGCGCCCGGCTGCCCGCTATCCATCCGCAGCTGCAAGTGCTCGGGGGAGCGGCCCAAGG
GGCTGAGCGGCGGCGTCCCTGGCCCGGCTCGGCGGAGGGTGGTGTGCAGCGGCGGGGACCTCCCGGAG
CCTCCCGAGCCCGGCCTTCTGCCTAACGGCACCGTTACCCTTGACCTGAGGAACAACATCATCAGCAC
AGTGCAGCCGGGCGCCTTCCTGGGCCTGGGGGAGCTGAAGCGTTTAGATCTCTCCAACAACCGGATTG
GCTGTCTCACCTCCGAGACCTTCCAGGGCCTCCCCAGGCTTCTCCGACTAAACATATCTGGAAACATC
TTCTCCAGTCTGCAACCTGGGGTCTTTGATGAGCTGCCAGCCCTTAAGGTTGTGGACTTGGGCACCGA
GTTCCTGACCTGTGACTGCCACCTGCGCTGGCTGCTGCCCTGGGCCCAGAATCGCTCCCTGCAGCTGT
CGGAACACACGCTCTGTGCTTACCCCAGTGCCCTGCATGCTCATGCCCTGGGCAGCCTCCAGGAGGCC
CAGCTCTGCTGCGAGGGGGCCCTGGAGCTGCACACACACCACCTCATCCCGTCCCTACGCCAAGTGGT
GTTCCAGGGGGATCGGCTGCCCTTCCAGTGCTCTGCCAGCTACCTGGGCAACGACACCCGCATCCGCT
GGTACCACAACCGAGCCCCTGTGGAGGGTGATGAGCAGGCGGGCATCCTCCTGGCCGAGAGCCTCATC
CACGACTGCACCTTCATCACCAGTGAGCTGACGCTGTCTCACATCGGCGTGTGGGCCTCAGGCGAGTG
GGAGTGCACCGTGTCCATGGCCCAAGGCAACGCCAGCAAGAAGGTGGAGATCGTGGTGCTGGAGACCT
CTGCCTCCTACTGCCCCGCCGAGCGTGTTGCCAACAACCGCGGGGACTTCAGGTGGCCCCGAACTCTG
GCTGGCATCACAGCCTACCAGTCCTGCCTGCAGTATCCCTTCACCTCAGTGCCCCTGGGCGGGGGTGC
CCCGGGCACCCGAGCCTCCCGCCGGTGTGACCGTGCCGGCCGCTGGGAGTCAGGGGACTACTCCCACT
GTCTCTACACCAACGACATCACCAGGGTGCTGTACACCTTCGTGCTGATGCCCATCAATGCCTCCAAT
GCGCTGACCCTGGCTCACCAGCTGCGCGTGTACACAGCCGAGGCCGCTAGCTTTTCAGACATGATGGA
TGTAGTCTATGTGGCTCAGATGATCCAGAAATTTTTGGGTTATGTCGACCAGATCAAAGAGCTGGTAG
AGGTGATGGTGGACATGGCCAGCAACTTGATGCTGGTGGACGAGCACCTGCTGTGGCTGGCCCAGCGC
GAGGACAAGGCCTGCAGCCGCATCGTGGGTGCCCTGGAGCGCATTGGGGGGGCCGCCCTCAGCCCCCA
TGCCCAGCACATCTCAGTGAATGCGAGGAACGTGGCATTGGAGGCCTACCTCATCAAGCCGCACAGCT
ACGTGGGCCTGACCTGCACAGCCTTCCAGAGGAGGGAGGGAGGGGTGCCGGGCACACGGCCAGGAAGC
CCTGGCCAGAACCCCCCACCTGAGCCCGAGCCCCCAGCTGACCAGCAGCTCCGCTTCCGCTGCACCAC
CGGGAGGCCCAATGTTTCTCTGTCGTCCTTCCACATCAAGAACAGCGTGGCCCTGGCCTCCATCCAGC
TGCCCCCGAGTCTATTCTCATCCCTTCCGGCTGCCCTGGCTCCCCCGGTGCCCCCAGACTGCACCCTG
CAACTGCTCGTCTTCCGAAATGGCCGCCTCTTCCACAGCCACAGCAACACCTCCCGCCCTGGAGCTGC
TGGGCCTGGCAAGAGGCGTGGCGTGGCCACCCCCGTCATCTTCGCAGGAACCAGTGGCTGTGGCGTGG
GAAACCTGACAGAGCCAGTGGCCGTTTCGCTGCGGCACTGGGCTGAGGGAGCCGAACCTGTGGCCGCT
TGTTGGAGCCAGGAGGGGCCCGGGGAGGCTGGGGGCTGGACCTCGGAGGGCTGCCAGCTCCGCTCCAG
CCAGCCCAATGTCAGCGCCCTGCACTGCCAGCACTTGGGCAATGTGGCCGTGCTCATGGAGCTGAGCG
CCTTTCCCAGGGAGGTGGGGGGCGCCGGGGCAGGGCTGCACCCCGTGGTATACCCCTGCACGGCCTTG
CTGCTGCTCTGCCTCTTCGCCACCATCATCACCTACATCCTCAACCACAGCTCCATCCGTGTGTCCCG
GAAAGGCTGGCACATGCTGCTGAACTTGTGCTTCCACATAGCCATGACCTCTGCTGTCTTTGCGGGGG
GCATCACACTCACCAACTACCAGATGGTCTGCCAGGCGGTGGGCATCACCCTGCACTACTCCTCCCTA
TCCACGCTGCTCTGGATGGGCGTGAAGGCGCGAGTGCTCCATAAGGAGCTCACCTGGAGGGCACCCCC
TCCGCAAGAAGGGGACCCCGCTCTGCCTACTCCCAGTCCTATGCTCCGGTACATACTTTCAATTCCAG
CTTTGCAATTGGGGAGGGACTCCAACGCAGGCGTAGGAAACCTCCCAAGGTTCTATTTGATCGCTGGA
GGGATTCCACTCATTATCTGTGGCATCACAGCTGCAGTCAACATTCACAACTACCGGGACCACAGCCC
CTACTGCTGGCTGGTGTGGCGTCCAAGCCTTGGCGCCTTCTACATCCCTGTGGCTTTGATTCTGCTCA
TCACCTGGATCTATTTCCTGTGCGCCGGGCTACGCTTACGGGGTCCTCTGGCACAGAACCCCAAGGCG
GGCAACAGCAGGGCCTCCCTGGAGGCAGGGGAGGAGCTGAGGGGTTCCACCAGGCTCAGGGGCAGCGG
CCCCCTCCTGAGTGACTCAGGTTCCCTTCTTGCTACTGGGAGCGCGCGAGTGGGGACGCCCGGGCCCC
CGGAGGATGGTGACAGCCTCTATTCTCCGGGAGTCCAGCTAGGGGCGCTGGTGACCACGCACTTCCTG
TACTTGGCCATGTGGGCCTGCGGGGCTCTGGCAGTGTCCCAGCGCTGGCTGCCCCGGGTGGTGTGCAG
CTGCTTGTACGGGGTGGCAGCCTCCGCCCTGGGCCTCTTCGTCTTCACTCACCACTGTGCCAGGCGGA
GGGACGTGAGAGCCTCGTGGCGCGCCTGCTGCCCCCCTGCCTCTCCCGCGGCCCCCCATGCCCCGCCC
CGGGCCCTGCCCGCCGCCGCAGAGGACGGTTCCCCGGTGTTCGGGGAGGGGCCCCCCTCCCTCAAGTC
CTCCCCAAGCGGCAGCAGCGGCCATCCGCTGGCTCTGGGCCCCTGCAAGCTCACCAACCTGCAGCTGG
CCCAGAGTCAGGTGTGCGAGGCGGGGGCGGCGGCCGGCGGGGAAGGAGAGCCGGAGCCGGCGGGCACC
CGGGGAAACCTCGCCCACCGCCACCCCAACAACGTGCACCACGGGCGTCGGGCGCACAAGAGCCGGGC
CAAGGGACACCGCGCGGGGGAGGCCTGCGGCAAGAACCGGCTCAAGGCCCTGCGCGGGGGCGCGGCGG
GGGCGCTGGAACTGCTGTCCAGCGAGAGCGGTAGTCTGCACAACAGCCCCACCGACAGCTACCTGGGC
AGCAGCCGCAACAGCCCGGGCGCCGGCCTGCAGCTGGAAGGCGAGCCCATGCTCACGCCGTCCGAGGG
CAGCGACACCAGCGCCGCGCCGCTTTCTGAGGCGGGCCGGGCAGGCCAGCGCCGCAGCGCCAGCCGCG
ACAGTCTCAAGGGCGGCGGCGCGCTGGAGAAGGAGAGCCATCGCCGCTCGTACCCGCTCAACGCCGCC
AGCCTAAACGGCGCCCCCAAGGGGGGCAAGTACGACGACGTCACCCTGATGGGCGCGGAGGTAGCCAG
CGGCGGCTGCATGAAGACCGGACTCTGGAAGAGCGAAACTACCGTCTAAGGTGGGGCGGGCGACGCGG
TAGACGGGTTGGCCACGCGGCTCGTTCCCCCGCTCCTCGGGGCCCTCCAAGGTGTCTCCGTAGTCAGC
AGGTTGGAGGCAGAGGAGCCGAT

| | | | |
|---|---|---|---|
| NOV10b, CG50925-08 | SEQ ID NO: 260 | 1330 aa | MW at 141782.5kD |
| Protein Sequence | | | |

MRGAPARLLLPLLPWLLLLLAPEARGAPGCPLSIRSCKCSGERPKGLSGGVPGPARRRVVCSGGDLPE
PPEPGLLPNGTVTLDLRNNIISTVQPGAFLGLGELKRLDLSNNRIGCLTSETFQGLPRLLRLNISGNI
FSSLQPGVFDELPALKVVDLGTEFLTCDCHLRWLLPWAQNRSLQLSEHTLCAYPSALHAHALGSLQEA
QLCCEGALELHTHHLIPSLRQVVFQGDRLPFQCSASYLGNDTRIRWYHNRAPVEGDEQAGILLAESLI
HDCTFITSELTLSHIGVWASGEWECTVSMAQGNASKKVEIVVLETSASYCPAERVANNRGDFRWPRTL
AGITAYQSCLQYPFTSVPLGGGAPGTRASRRCDRAGRWESGDYSHCLYTNDITRVLYTFVLMPINASN
ALTLAHQLRVYTAEAASFSDMMDVVYVAQMIQKFLGYVDQIKELVEVMVDMASNLMLVDEHLLWLAQR
EDKACSRIVGALERIGGAALSPHAQHISVNARNVALEAYLIKPHSYVGLTCTAFQRREGGVPGTRPGS
PGQNPPPEPEPPADQQLRFRCTTGRPNVSLSSFHIKNSVALASIQLPPSLFSSLPAALAPPVPPDCTL
QLLVFRNGRLFHSHSNTSRPGAAGPGKRRGVATPVIFAGTSGCGVGNLTEPVAVSLRHWAEGAEPVAA
WWSQEGPGEAGGWTSEGCQLRSSQPNVSALHCQHLGNVAVLMELSAFPREVGGAGAGLHPVVYPCTAL
LLLCLFATIITYILNHSSIRVSRKGWHMLLNLCFHIAMTSAVFAGGITLTNYQMVCQAVGITLHYSSL
STLLWMGVKARVLHKELTWEAPPPQEGDPALPTPSPMLRYILSIPALQLGRDSNAGVGNLPRFYLIAG
GIPLIICGITAAVNIHNYRDHSPYCWLVWRPSLGAFYIPVALILLITWIYFLCAGLRLRGPLAQNPKA
GNSRASLEAGEELRGSTRLRGSGPLLSDSGSLLATGSARVGTPGPPEDGDSLYSPGVQLGALVTTHFL
YLAMWACGALAVSQRWLPRVVCSCLYGVAASALGLFVFTHHCARRRDVRASWRACCPPASPAAPHAPP
RALPAAAEDGSPVFGEGPPSLKSSPSGSSGHPLALGPCKLTNLQIAQSQVCEAGAAAGGEGEPEPAGT
RGNLAHRHPNNVHHGRRAHKSRAKGHRAGEACGKNRLKALRGGAAGALELLSSESGSLHNSPTDSYLG
SSRNSPGAGLQLEGEPMLTPSEGSDTSAAPLSEAGRAGQRRSASRDSLKGGGALEKESHPRSYPLNAA
SLNGAPKGGKYDDVTLMGAEVASGGCMKTGLWKSETTV

| | | |
|---|---|---|
| NOV10c, 226990087 | SEQ ID NO: 261 | 341 bp |
| DNA Sequence | ORF Start: at 3 | ORF Stop: end of sequence |

CAACCGGATCCGAGCTGAAGCGTTTAGATCTCTCCAACAACCGGATTGGCTGTCTCACCTCCGAGACC
TTCCAGGGCCTCCCCAGGCTTCTCCGACTAAACATATCTGGAAACATCTTCTCCAGTCTGCAACCTGG
GGTCTTTGATGAGCTGCCAGCCCTTAAGGTTGTGGACTTGGGCACCGAGTTCCTGACCTGTGACTGCC
ACCTGCGCTGGCTGCTGCCCTGGGCCCAGAATCGCTCCCTGCAGCTGTCGGAACACACGCTCTGTGCT
TACCCCAGTGCCCTGCATGCTCAGGCCCTGGGCAGCCTCCAGGAGGCCCAGCTCTGCTGCGAGCTCGA
G

| | | | |
|---|---|---|---|
| NOV10c, 226990087 | SEQ ID NO: 262 | 113 aa | MW at 12539.2kD |
| Protein Sequence | | | |

TGSELKRLDLSNNRIGCLTSETFQGLPRLLRLNISGNIFSSLQPGVFDELPALKVVDLGTEFLTCDCH
LRWLLPWAQNRSLQLSEHTLCAYPSALHAQALGSLQEAQLCCELE

| | | |
|---|---|---|
| NOV10d, 320054488 | SEQ ID NO: 263 | 2227 bp |
| DNA Sequence | ORF Start: at 2 | ORF Stop: end of sequence |

CACCGGATCCGCGCCCGGCTGCCCGCTATCCATCCGCAGCTGCAAGTGCTCGGGGGAGCGGGCCAAGG
GGCTGAGCGGCGGCGTCCCTGGCCCGGCTCGGCGGAGGGTGGTGTGCAGCGGCGGGGACCTCCCGGAG
CCTCCCGAGCCCGGCCTTCTGCCTAACGGCACCGTTACCCTGCTCTTGAGCAATAACAAGATCACGGG
GCTCCGCAATGGCTCCTTCCTGGGACTGTCACTGCTGGAGAAGCTGGACCTGAGGAACAACATCATCA
GCACAGTGCAGCCGGGCGCCTTCCTGGGCCTGGGGGAGCTGAAGCGTTTAGATCTCTCCAACAACCGG
ATTGGCTGTCTCACCTCCGAGACCTTCCAGGGCCTCCCCAGGCTTCTCCGACTAAACATATCTGGAAA
CATCTTCTCCAGTCTGCAACCTGGGGTCTTTGATGAGCTGCCAGCCCTTAAGGTTGTGGACTTGGGCA
CCGAGTTCCTGACCTGTGACTGCCACCTGCGCTGGCTGCTGCCCTGGGCCCAGAATCGCTCCCTGCAG
CTGTCGGAACACACGCTCTGTGCTTACCCCAGTGCCCTGCATGCTCAGGCCCTGGGCAGCCTCCAGGA
GGCCCAGCTCTGCTGCGAGGGGGCCCTGGAGCTGCACACACACCACCTCATCCCGTCCCTACGCCAAG
TGGTGTTCCAGGGGGATCGGCTGCCCTTCCAGTGCTCTGCCAGCTACCTGGGCAACGACACCCGCATC
CGCTGGTACCACGACCGAGCCCCTGTGGAGGGTGATGAGCAGGCGGGCATCCTCCTGGCCGAGAGCCT
CATCCACGACTGCACCTTCATCACCAGTGAGCTGACGCTGTCTCACATCGGCGTGTGGGCCTCAGGCG
AGTGGGAGTGCACCGTGTCCATGGCCCAAGGCAACGCCAGCAAGAAGGTGGAGATCGTGGTGCTGGAG
ACCTCTGCCTCCTACTGCCCCGCCGAGCGTGTTGCCAACAACCGCGGGGACTTCAGGTGGCCCCGAAC
TCTGGCTGGCATCACAGCCTACCAGTCCTGCCTGCAGTATCCCTTCACCTCAGTGCCCCTGGGCGGGG
GTGCCCCGGGCACCCGAGCCTCCCGCCGGTGTGACCGTGCCGGCCGCTGGGAGCCAGGGGACTACTCC
CACTGTCTCTACACCAACGACATCACCAGGGTGCTGTACACCTTCGTGCTGATGCCCATCAATGCCTC
CAATGCGCTGACCCTGGCTCACCAGCTGCGCGTGTACACAGCCGAGGCCGCTAGCTTTTCAGACATGA
TGGATGTAGTCTATGTGGCTCAGATGATCCAGAAATTTTTGGGTTATGTCGACCAGATCAAAGAGCTG
GTAGAGGTGATGGTGGACATGGCCAGCAACCTGATGCTGGTGGACGAGCACCTGCTGTGGCTGGCCCA
GCGCGAGGACAAGGCCTGCAGCCGCATCGTGGGTGCCCTGGAGCGCATTGGGGGGGCCGCCCTCAGCC
CCCATGCCCAGCACATCTCAGTGAATGCGAGGAACGTGGCATTGGAGGCCTACCTCATCAAGCCGCAC
AGCTACGTGGGCCTGACCTGCACAGCCTTCCAGAGGAGGGAGGGAGGGGTGCCGGGCACACGGCCAGG
AAGCCCTGGCCAGAACCCCCCACCTGAGCCCGAGCCCCCAGCTGACCAGCAGCTCCGCTTCCGCTGCA
CCACCGGGAGGCCCAATGTTTCTCTGTCGTCCTTCCACATCAAGAACAGCGTGGCCCTGGCCTCCATC
CAGCTGCCCCCGAGTCTATTCTCATCCCTTCCGGCTGCCCTGGCTCCCCCGGTGCCCCCAGACTGCAC
CCTGCAACTGCTCGTCTTCCGAAATGGCCGCCTCTTCCACAGCCACAGCAACACCTCCCGCCCTGGAG
CTGCTGGGCCTGGCAAGAGGCGTGGCGTGGCCACCCCCGTCATCTTCGCAGGAACCAGTGGCTGTGGC
GTGGGAAACCTGACAGAGCCAGTGGCCGTTTCGCTGCGGCACTGGGCTGAGGGAGCCGAACCTGTGGC
CGCTTGGTGGAGCCAGGAGGGGCCCGGGGAGGCTGGGGGCTGGACCTCGGAGGGCTGCCAGCTCCGCT
CCAGCCAGCCCAATGTCAGCGCCCTGCACTGCCAGCACTTGGGCAATGTGGCCGTGCTCATGGAGCTG
AGCGCCTTTCCCAGGGAGGTGGGGGGCGCCGGGGCAGGGCTGCTCGAGGGC

| | | | |
|---|---|---|---|
| NOV10d, 320054488 | SEQ ID NO: 264 | 742 aa | MW at 79823.1kD |
| Protein Sequence | | | |

TGSAPGCPLSIRSCKCSGEPAKGLSGGVPGPARRRVVCSGGDLPEPPEPGLLPNGTVTLLLSNNKITG
LRNGSFLGLSLLEKLDLRNNIISTVQPGAFLGLGELKRLDLSNNRIGCLTSETFQGLPRLLRLNISGN
IFSSLQPGVFDELPALKVVDLGTEFLTCDCHLRWLLPWAQNRSLQLSEHTLCAYPSALHAQALGSLQE
AQLCCEGALELHTHHLIPSLRQVVFQGDRLPFQCSASYLGNDTRIRWYHDRAPVEGDEQAGILLAESL
IHDCTFITSELTLSHIGVWASGEWECTVSMAQGNASKKVEIVVLETSASYCPAERVANNRGDFRWPRT
LAGITAYQSCLQYPFTSVPLGGGAPGTRASRRCDPAGRWEPGDYSHCLYTNDITRVLYTFVLMPINAS
NALTLAHQLRVYTAEAASFSDMMDVVYVAQMIQKFLGYVDQIKELVEVMVDMASNLMLVDEHLLWLAQ
REDKACSRIVGALERIGGAALSPHAQHISVNARNVALEAYLIKPHSYVGLTCTAFQRREGGVPGTRPG
SPGQNPPPEPEPPADQQLRFRCTTGRPNVSLSSFHIKNSVALASIQLPPSLFSSLPAALAPPVPPDCT
LQLLVFRNGRLFHSHSNTSRPGAAGPGKRRGVATPVIFAGTSGCGVGNLTEPVAVSLRHWAEGAEPVA
AWWSQEGPGEAGGWTSEGCQLRSSQPNVSALHCQHLGNVAVLMELSAFPREVGGAGAGLLEG

| | | |
|---|---|---|
| NOV10e, CG50925-02 | SEQ ID NO: 265 | 7860 bp |
| DNA Sequence | ORF Start: ATG at 1 | ORF Stop: TAA at 4627 |

ATGCGGGGGGCGCCCGCGCGCCTGCTGCTGCCGCTGCTGCCGTGGCTCCTGCTGCTCCTGGCGCCCGA
GGCTCGGGGCGCGCCCGGCTGCCCGCTATCCATCCGCAGCTGCAAGTGCTCGGGGGAGCGGCCCAAGG
GGCTGAGCGGCGGCGTCCCTGGCCCGGCTCGGCGGAGGGTGGTGTGCAGCGGCGGGGACCTCCCGGAG
CCTCCCGAGCCCGGCCTTCTGCCTAACGGCACCGTTACCCTTGGCAACCGTTTCTCCACTGCCCCTTG
CCCGCAGTCGGGGCTGGCCCAGTCTTTTCGATTGTGTCCAGAACAATACAGTAACGCCTCTGGTTGGG
TGAGTTCAGAGGCTGCCCGCTTCCTGACAGGACACAACTGGTCAGCCTCTGACGCTGGCCGCCCGGCC
TGCATTTTCCCCGGCCCGGTCTCCTGGAGACAGGTACCAGCTTACAACGCCGGACAGGATCAGGGTAG
CAAATATAGCTTCTATGGAGGAAAGGTCAAAGATGAGTATCTGCGTAGGTCCCCGATCTGTCGCTCTG
CTGCCGCCCTGCACACTGACACCAGTGCCCGCAGCTGGTCTGAATTTAGGCTCTTGAGCAATAACAAG
ATCACGGGGCTCCGCAATGGCTCCTTCCTGGGACTGTCACTGCTGGAGAAGCTTCTCATGAGAGCACA
TTTTTCTGATGAAGATGAAAAGACGGCCTGTCCTTCTCCAGCACCAAGGCTCTGGAGGCTGCATAGCA
AGTGCACCCTGAGCATGGCAGGCAATCAGAAGAACCAGGGCGGCAGCAGCTCACAAGAGGCAGCATCT
GGCTTGATGCCGAGTCGGCGGCTCCAGCGGGGAGAGGTTGAGGGCCTCTGCACTTGCCTCTGCACGGA
CCTGAGGAACAACATCATCAGCACAGTGCAGCCGGGCGCCTTCCTGGGCCTGGGGGAGCTGAAGCGTT
TAGATCTCTCCAACAACCGGATTGGCTGTCTCACCTCCGAGACCTTCCAGGGCCTCCCCAGGCTTCTC
CGACTAAACATATCTGGAAACATCTTCTCCAGTCTGCAACCTGGGGTCTTTGATGAGCTGCCAGCCCT
TAAGGTTGTGGACTTGGGCACCGAGTTCCTGACCTGTGACTGCCACCTGCGCTGGCTGCTGCCCTGGG
CCCAGAATCGCTCCCTGCAGCTGTCGGAACACACGCTCTGTGCTTACCCCAGTGCCCTGCATGCTCAT
GCCCTGGGCAGCCTCCAGGAGGCCCAGCTCTGCTGCGAGGGGGCCCTGGAGCTGCACACACACCACCT
CATCCCGTCCCTACGCCAAGTGGTGTTCCAGGGGGATCGGCTGCCCTTCCAGTGCTCTGCCAGCTACC
TGGGCAACGACACCCGCATCCGCTGGTACCACAACCGAGCCCCTGTGGAGGGTGATGAGCAGGCGGGC
ATCCTCCTGGCCGAGAGCCTCATCCACGACTGCACCTTCATCACCAGTGAGCTGACGCTGTCTCACAT
CGGCGTGTGGGCCTCAGGCGAGTGGGAGTGCACCGTGTCCATGGCCCAAGGCAACGCCAGCAAGAAGG
TGGAGATCGTGGTGCTGGAGACCTCTGCCTCCTACTGCCCCGCCGAGCGTGTTGCCAACAACCGCGGG
GACTTCAGGTGGCCCCGAACTCTGGCTGGCATCACAGCCTACCAGTCCTGCCTGCAGTATCCCTTCAC
CTCAGTGCCCCTGGGCGGGGGTGCCCCGGGCACCCGAGCCTCCCGCCGGTGTGACCGTGCCGGCCGCT
GGGAGCCAGGGGACTACTCCCACTGTCTCTACACCAACGACATCACCAGGGTGCTGTACACCTTCGTG
CTGATGCCCATCAATGCCTCCAATGCGCTGACCCTGGCTCACCAGCTGCGCGTGTACACAGCCGAGGC
CGCTAGCTTTTCAGACATGATGGATGTAGTCTATGTGGCTCAGATGATCCAGAAATTTTTGGGTTATG
TCGACCAGATCAAAGAGCTGGTAGAGGTGATGGTGGACATGCCCAGCAACTTGATGCTGGTGGACGAG
CACCTGCTGTGGCTGGCCCAGCGCGAGGACAAGGCCTGCAGCCGCATCGTGGGTGCCATAGAGCGCAT
TGGGGGGGCCGCCCTCAGCCCCCATGCCCAGCACATCTCAGTGAATGCGAGGAACGTGGCATTGGAGG
CCTACCTCATCAAGCCGCACAGCTACGTGGGCCTGACCTGCACAGCCTTCCAGAGGAGGGAGGGAGGG
GTGCCGGGCACACGGCCAGGAAGCCCTGGCCAGAACCCCCCACCTGAGCCCGAGCCCCCAGCTGACCA
GCAGCTCCGCTTCCGCTGCACCACCGGGAGGCCCAATGTTTCTCTGTCGTCCTTCCACATCAAGAACA
GCGTGGCCCTGGCCTCCATCCAGCTGCCCCCGAGTCTATTCTCATCCCTTCCGGCTGCCCTGGCTCCC
CCGGTGCCCCCAGACTGCACCCTGCAACTGCTCGTCTTCCGAAATGGCCGCCTCTTCCACAGCCACAG
CAACACCTCCCGCCCTGGAGCTGCTGGGCCTGGCAAGAGGCGTGGCGTGGCCACCCCCGTCATCTTCG
CAGGAACCAGTGGCTGTGGCGTGGGAAACCTGACAGAGCCAGTGGCCGTTTCGCTGCGGCACTGGGCT
GAGGGAGCCGAACCTGTGGCCGCTTGGTGGAGCCAGGAGGGGCCCGGGGAGGCTGGGGGCTGGACCTC
GGAGGGCTGCCAGCTCCGCTCCAGCCAGCCCAATGTCAGCGCCCTGCACTGCCAGCACTTGGGCAATG
TGGCCGTGCTCATGGAGCTGAGCGCCTTTCCCAGGGAGGTGGGGGGCGCCGGGGCAGGGCTGCACCCC
GTGGTATACCCCTGCACGGCCTTGCTGCTGCTCTGCCTCTTCGCCACCATCATCACCTACATCCTCAA
CCACAGCTCCATCCGTGTGTCCCGGAAAGGCTGGCACATGCTGCTGAACTTGTGCTTCCACATAGCCA
TGACCTCTGCTGTCTTTGCGGGGGGCATCACACTCACCAACTACCAGATGGTCTGCCAGGCGGTGGGC
ATCACCCTGCACTACTCCTCCCTATCCACGCTGCTCTGGATGGGCGTGAAGGCGCGAGTGCTCCATAA
GGAGCTCACCTGGAGGGCACCCCCTCCGCAAGAAGGGGACCCCGCTCTGCCTACTCCCAGTCCTATGC
TCCGGTACATACTTTCAATTCCAGCTTTGCAATTGGGGAGGGACTCCAACGCAGGCGTAGGAAACCTC
CCAAGGTTCTATTTGATCGCTGGAGGGATTCCACTCATTATCTGTGGCATCACAGCTGCAGTCAACAT
CCACAACTACCGGGACCACAGCCCCTACTGCTGGCTGGTGTGGCGTCCAAGCCTTGGCGCCTTCTACA
TCCCTGTGGCTTTGATTCTGCTCATCACCTGGATCTATTTCCTGTGCGCCGGGCTACGCTTACGGGGT
CCTCTGGCACAGAACCCCAAGGCGGGCAACAGCAGGGCCTCCCTGGAGGCAGGGGAGGAGCTGAGGGG
TTCCACCAGGCTCAGGGGCAGCGGCCCCCTCCTGAGTGACTCAGGTTCCCTTCTTGCTACTGGGAGCG
CGCGAGTGGGGACGCCCGGGCCCCCGGAGGATGGTGACAGCCTCTATTCTCCGGGAGTCCAGCTAGGG
GCGCTGGTGACCACGCACTTCCTGTACTTGGCCATGTGGGCCTGCGGGGCTCTGGCAGTGTCCCAGCG
CTGGCTGCCCCGGGTGGTGTGCAGCTGCTTGTACGGGGTGGCAGCCTCCGCCCTGGGCCTCTTCGTCT
TCACTCACCACTGTGCCAGGCGGAGGGACGTGAGAGCCTCGTGGCGCGCCTGCTGCCCCCCTGCCTCT
CCCGCGGCCCCCCATGCCCCGCCCCGGGCCCTGCCCGCCGCCGCAGAGGACGGTTCCCCGGTGTTCGG
GGAGGGGCCCCCCTCCCTCAAGTCCTCCCCAAGCGGCAGCAGCGGCCATCCGCTGGCTCTGGGCCCCT
GCAAGCTCACCAACCTGCAGCTGGCCCAGAGTCAGGTGTGCGAGGCGGGGGCGGCGGCCGGCGGGGAA
GGAGAGCCGGAGCCGGCGGGCACCCGGGGAAACCTCGCCCACCGCCACCCCAACAACGTGCACCACGG
GCGTCGGGCGCACAAGAGCCGGGCCAAGGGACACCGCGCGGGGGAGGCCTGCGGCAAGAACCGGCTCA
AGGCCCTGCGCGGGGGCGCGGCGGGGGCGCTGGAACTGCTGTCCAGCGAGAGCGGTAGTCTGCACAAC
AGCCCCACCGACAGCTACCTGGGCAGCAGCCGCAACAGCCCGGGCGCCGGCCTGCAGCTGGAAGGCGA
GCCCATGCTCACGCCGTCCGAGGGCAGCGACACCAGCGCCGCGCCGCTTTCTGAGGCGGGCCGGGCAG
GCCAGCGCCGCAGCGCCAGCCGCGACAGTCTCAAGGGCGGCGGCGCGCTGGAGAAGGAGAGCCATCGC
CGCTCGTACCCGCTCAACGCCGCCAGCCTAAACGGCGCCCCCAAGGGGGGCAAGTACGACGACGTCAC
CCTGATGGGCGCGGAGGTAGCCAGCGGCGGCTGCATGAAGACCGGACTCTGGAAGAGCGAAACTACCG
TCTAAGGTGGGGCGGGCGACGCGGTAGACGGGTTGGCCACGCGGCTCGTTCCCCCGCTCCTCGGGGCC
CTCCAAGGTGTCTCCGTAGTCAGCAGGTTGGAGGCAGAGGAGCCGATGGCTGGAGGAAGCCCACAGGC
GGATGTTCCCCACTTGCCTAGAGGGCATCCCTCTGGGGTAGCGACAGACAATCCCAGAAACACGCATA
ATACATTTCCGTCCAGCCCGGGGCAGTCTGACTGTCGGTGCCCTCCCAGGAACGGGGAAGGCCTCCGT
CTGTGTGAAAGGGCACAGCACATCCCAGGTGCACCCTCCCCAAGTACTCCCACCCCGCCTACTGTCCA
TGCGGCCTCACTGGGGGCCATCAGCCTCACCAGCAAAGCAGAGATGAGAGCGTGGGAACTGTGTTCTT
TCCTCCCTGCCCTCTACTGATTTCAGCCCAGCCCCTGCCTAGATCCTAGGTCCCTTTTCCTCCCGAGT
TTGGCTGGCACGAGAGCTAGCCCAGCACATGAAGCAGGTGATGTTAAGTCACAAGGTGCTGCTTTTCA
GATCCACTATGCAAGAGGGGAGGGTGGGGCCACGTGAAAGGCAGCTCTAGACATCAACCAGTCCTGGG
GGAGGGGAGTGGGAACCGGGCACAACTAGGAACAATGCCACCATTCCCACAGGAGTGGTACTTAAACC
AGACAGCAGGGTTCAGAGGTGGCACACCGGGACAAAGCTGAGGCCCTGCACCTCAACAGCTGACTGCC
AGGTGCCTGTGGGTGAACTGAGGGGAGTAGAGGGAGAGGGCAGGTGGAACTGGGGCAGAATCTAGTCA
TGCCCTAAAGCTAGTCCTGTAAACAATGGTGCCCCAGAAAGCTGCAGGTGGTGTTTGGAGAAGCAGTT
ACTTTTCAGTTACAAGACCCATCTCCCTAGTCTCAGCCTTACAACACCACGGGACTAAGGAAGAGCAC
TTCCTTGCCTCCGTAAGGCCAGAGGAAGAACCATCCCAATCATTTGATCTCCAGCTCCACAGTAGAGA
GAAACCTACAAAATGTCAAACCAGCTTCCCGACTCCCAGGAGCTCAAGCCAAGCCCAGAGGCAGTGGC
TGGGGTCCCTGCAGGTCATGAGGGGCCTATGCCTTTACTCCTTTTAAACACCAGCACCCGTCTTTTCC
CCAACCTAAAACCAACCACCAGCATTTCACTACAGGACCAAATGGAAACCGAGGGAACCCTGGGTCTT
GGGAAGAACAACAGGAAACCAAGGTCTGACCTAGGGTTCCCTCCCAGTCTTCACATCACTCTGGCCTC
ATCACCAAGGTGACAGAGGACACAGGGGAGGGGGAAAACCCACACACACTCCTTGGAATGGGTCCTGT
TATTTATGCTTGCTGCACAGACATATTAGAAGAAAAAAAAAAAGCTTTGTATTATTCTTCCACATATG
CTGGCTGCTGTTTACACACCCTGCCAATGCCTTAGCACTGGAGAGCTTTTTGCAATATGCTGGGGAAA
GGGGAGGGAGGGAATGAAAGTGCCAAAGAAAACATGTTTTTAAGAACTCGGGTTTTATACAATAGAAT
GTTTTCTAGCAGATGCCTCTTGTTTTAATATATTAAAATTTTGCAAAGCCCTTTGAGCTACTGCCTTA
GTCTACCCACTGTCCTTTTGTTATGAGGTAGAGGATCTCATGACACCATACACACAAACCCATCATTG
CCTGTGAATGCACGTAGGGCCAGAATTCCCCAGTTCCCGCTCCTCTGAGGGTTGATACTGCTGGGAAT
GCCAACCACTCCACAAGCAGAGGGAAGCCCCCTCAGGCCTGCAGGAGGAGCCGCAGCAGTGTGTCCAA
TTCAAACCAGCAGCAAAGAGCCTGACATTTTCCCATCCATCTATGAGGAAAGCCATCTCACAGAACAT
GGACATAGGCAACTTGCTCTCCCACACCAAGGGATGGGAATCTCTCCTACCTATAGTCATCCCTGCAC
TCCTGACTTTACTCCAGGACCCAGGGTCCAACTAATGGCAGAGCCCCTCTTGGTTCCTTCAAACAAGA
AAAGCAATACCTACGGACTGGTGTACACTTCCATCCTTGGTTATAACAGGAATGTTATCAAGCTGTCA
GAACAGGATGAAGTGCTCCCAGTGGATATCCATCAGGGAGGGTTAGGGACACTCGTGGCAGCCTGTCT
AGCAGCCTGGGCTCTCTGAAAGTCCCTAACTTCCTGAGGGGTACGCAAATACTGTTCTATTTCACTAT
CAGAAATGTTCTCATCTCCAGTGACAGTGGAGACAGGGGGTACAGGGCAGATCCGCTTCGGGGACTTC
AACATGCAGGGTGGCAAGAGAAGGGCAGGACTGGCCGGCCGCTTCCCCTGGGGTAAACCTAAGGAATT
ATTTCCCACCTCCCCTTCTCCTTGCCCCTGTCCCCACCCCGGTGGCTCCTTCTCTCGGGTCTCCACTT
CTGCTGTCCCATCCCGAAAGGCAGAGCGGACCAGTGACTGGCGGTGCTGGAGAAGGTCACCGATGTGC
TTCACCACAGACCGTTTGTCAAGTCTCAGAACTCGTAACCAGGCCAGCTGCTCAGCCATCCGCAGCAG
CACAGCCAGCAGCTCCTGCAGGCGGGAGGACGCCGGGTAGGGCAGGTCCACATTTGCCAATTTACAAA
ATCGGGCAAGGGAACATGAAAGCCGATCTGCAGGCTGCAGCGACTGCCAAGCCAGGAAAGTCGCAGCA
GTGATGACGGGCAAGGGATGCCTCCCGGTCACCAGCCACGTCTCATTTGCCAGCTCCACCAACTGCAT
TGTTCGAGACAGCATCTTCTCTTTGTCTTCCACGTATTTGGCTGGCACAGAAGGTGAAGCTTGGAACA
GTTTGAAGCTGAAATAACCAAAATGAGGGTTGGATCTTAATGATATAGGGGCTGCTCTCCCACAGTGA
GGAAAGACAGCCCACTCAAGATGGGGAAGCTATTCTGCCCTCAGGAATACTCAAGCTCACTGGGCAGC
AAGTTAATAAAGGTAGTGAGAGAAAACAGGGCGTCTTCCGCTTGTTAGGGGAAGGTGGAGGGATGGAG
GAGAGCACGAACATTTATTGGGCGCCTCCCAATCACCATTATTCTGAGTGCTTTACAACGTTCTCATT
TAATCTACGTGCACGTGCACCATCTTATGTGCATGTATAGTTAAAAAACTTTCCCATAGTCATCCAGC
CAGGCAGTAACCAAGCTTCAAATACAAGGCTATTTGACAC

| | | | |
|---|---|---|---|
| NOV10e, CG50925-02 | SEQ ID NO: 266 | 1542 aa | MW at 164901.2kD |
| Protein Sequence | | | |

MRGAPARLLLPLLPWLLLLLAPEARGAPGCPLSIRSCKCSGERPKGLSGGVPGPARRRVVCSGGDLPE
PPEPGLLPNGTVTLGNRFSTAPCPQSGLAQSFRLCPEQYSNASGWVSSEAARFLTGHNWSASDAGRPA
CIFPGPVSWRQVPAYNAGQDQGSKYSFYGGKVKDEYLRRSPICRSAAALHTDTSARSWSEFRLLSNNK
ITGLRNGSFLGLSLLEKLLMRAHFSDEDEKTACPSPAPRLWRLHSKCTLSMAGNQKNQGGSSSQEAAS
GLMPSRRLQRGEVEGLCTCLCTDLRNNIISTVQPGAFLGLGELKRLDLSNNRIGCLTSETFQGLPRLL
RLNISGNIFSSLQPGVFDELPALKVVDLGTEFLTCDCHLRWLLPWAQNRSLQLSEHTLCAYPSALHAH
ALGSLQEAQLCCEGALELHTHHLIPSLRQVVFQGDRLPFQCSASYLGNDTRIRWYHNRAPVEGDEQAG
ILLAESLIHDCTFITSELTLSHIGVWASGEWECTVSMAQGNASKKVEIVVLETSASYCPAERVANNRG
DFRWPRTLAGITAYQSCLQYPFTSVPLGGGAPGTRASRRCDRAGRWEPGDYSHCLYTNDITRVLYTFV
LMPINASNALTLAHQLRVYTAEAASFSDMMDVVYVAQMIQKFLGYVDQIKELVEVMVDMPSNLMLVDE
HLLWLAQREDKACSRIVGAIERIGGAALSPHAQHISVNARNVALEAYLIKPHSYVGLTCTAFQRREGG
VPGTRPGSPGQNPPPEPEPPADQQLRFRCTTGRPNVSLSSFHIKNSVALASIQLPPSLFSSLPAALAP
PVPPDCTLQLLVFRNGRLFHSHSNTSRPGAAGPGKRRGVATPVIFAGTSGCGVGNLTEPVAVSLRHWA
EGAEPVAAWWSQEGPGEAGGWTSEGCQLRSSQPNVSALHCQHLGNVAVLMELSAFPREVGGAGAGLHP
VVYPCTALLLLCLFATIITYILNHSSIRVSRKGWHMLLNLCFHIAMTSAVFAGGITLTNYQMVCQAVG
ITLHYSSLSTLLWMGVKARVLHKELTWRAPPPQEGDPALPTPSPMLRYILSIPALQLGRDSNAGVGNL
PRFYLIAGGIPLIICGITAAVNIHNYRDHSPYCWLVWRPSLGAFYIPVALILLITWIYFLCAGLRLRG
PLAQNPKAGNSRASLEAGEELRGSTRLRGSGPLLSDSGSLLATGSARVGTPGPPEDGDSLYSPGVQLG
ALVTTHFLYLAMWACGALAVSQRWLPRVVCSCLYGVAASALGLFVFTHHCARRRDVRASWPACCPPAS
PAAPHAPPRALPAAAEDGSPVFGEGPPSLKSSPSGSSGHPLALGPCKLTNLQLAQSQVCEAGAAAGGE
GEPEPAGTRGNLAHRBPNNVHHGRRAHKSRAKGHRAGEACGKNRLKALRGGAAGALELLSSESGSLHN
SPTDSYLGSSRNSPGAGLQLEGEPMLTPSEGSDTSAAPLSEAGRAGQRRSASRDSLKGGGALEKESHR
RSYPLNAASLNGAPKGGKYDDVTLMGAEVASGGCMKTGLWKSETTV

| | | |
|---|---|---|
| NOV10f, 2482O8844 | SEQ ID NO: 267 | 1888 bp |
| DNA Sequence | ORF Start: at 2 | ORF Stop: end of sequence |

CACCGGATCCGAGCTGAAGCGTTTAGATCTCTCCAACAACCGGATTGGCTGTCTCACCTCCGAGACCT
TCCAGGGCCTCCCCAGGCTTCTCCGACTAAACATATCTGGAAACATCTTCTCCAGTCTGCAACCTGGG
GTCTTTGATGAGCTGCCAGCCCTTAAGGTTGTGGACTTGGGCACCGAGTTCCTGACCTGTGACTGCCA
CCTGCGCTGGCTGCTGCCCTGGGCCCAGAATCGCTCCCTGCAGCTGTCGGAACACACGCTCTGTGCTT
ACCCCAGTGCCCTGCATGCTCAGGCCCTGGGCAGCCTCCAGGAGGCCCAGCTCTGCTGCGAGGGGGCC
CTGGAGCTGCACACACACCACCTCATCCCGTCCCTACGCCAAGTGGTGTTCCAGGGGGATCGGCTGCC
CTTCCAGTGCTCTGCCAGCTACCTGGGCAACGACACCCGCATCCGCTGGTACCACAACCGAGCCCCTG
TGGAGGGTGATGAGCAGGCGGGCATCCTCCTGGCCGAGAGCCTCATCCACGACTGCACCTTCATCACC
AGTGAGCTGACGCTGTCTCACATCGGCGTGTGGGCCTCAGGCGAGTGGGAGTGCACCGTGTCCATGGC
CCAAGGCAACGCCAGCAAGAAGGTGGAGATCGTGGTGCTGGAGACCTCTGCCTCCTACTGCCCCGCCG
AGCGTGTTGCCAACAACCGCGGGGACTTCAGGTGGCCCCGAACTCTGGCTGGCATCACAGCCTACCAG
TCCTGCCTGCAGTATCCCTTCACCTCAGTGCCCCTGGGCGGGGGTGCCCCGGGCACCCGAGCCTCCCG
CCGGTGTGACCGTGCCGGCCGCTGGGAGCCAGGGGACTACTCCCACTGTCTCTACACCAACGACATCA
CCAGGGTGCTGTACACCTTCGTGCTGATGCCCATCAATGCCTCCAATGCGCTGACCCTGGCTCACCAG
CTGCGCGTGTACACAGCCGAGGCCGCTAGCTTTTCAGACATGATGGATGTAGTCTATGTGGCTCAGAT
GATCCAGAAATTTTTGGGTTATGTCGACCAGATCAAAGAGCTGGTAGAGGTGATGGTGGACATGGCCA
GCAACCTGATGCTGGTGGACGAGCACCTGCTGTGGCTGGCCCAGCGCGAGGACAAGGCCTGCAGCCGC
ATCGTGGGTGCCCTGGAGCGCATTGGGGGGGCCGCCCTCAGCCCCCATGCCCAGCACATCTCAGTGAA
TGCGAGGAACGTGGCATTGGAGGCCTACCTCATCAAGCCGCACAGCTACGTGGGCCTGACCTGCACAG
CCTTCCAGAGGAGGGAGGGAGGGGTGCCGGGCACACGGCCAGGAAGCCCTGGCCAGAACCCCCCACCT
GAGCCCGAGCCCCCAGCTGACCAGCAGCTCCGCTTCCGCTGCACCACCGGGAGGCCCAATGTTTCTCT
GTCGTCCTTCCACATCAAGAACAGCGTGGCCCTGGCCTCCATCCAGCTGCCCCCGAGTCTATTCTCAT
CCCTTCCGGCTGCCCTGGCTCCCCCGGTGCCCCCAGACTGCACCCTGCAACTGCTCGTCTTCCGAAAT
GGCCGCCTCTTCCACAGCCACAGCAACACCTCCCGCCCTGGAGCTGCTGGGCCTGGCAAGAGGCGTGG
CGTGGCCACCCCCGTCATCTTCGCAGGAACCAGTGGCTGTGGCGTGGGAAACCTGACAGAGCCAGTGG
CCGTTTCGCTGCGGCACTGGGCTGAGGGAGCCGAACCTGTGGCCGCTTGGTGGAGCCAGGAGGGGCCC
GGGGAGGCTGGGGGCTGGACCTCGGAGGGCTGCCAGCTCCGCTCCAGCCAGCCCAATGTCAGCGCCCT
GCACTGCCAGCACTTGGGCAATGTGGCCGTGCTCATGGAGCTGAGCCTCGAG

| | | | |
|---|---|---|---|
| NOV10f, 248208844 | SEQ ID NO: 268 | 629 aa | MW at 68481.1kD |
| Protein Sequence | | | |

TGSELKRLDLSNNRIGCLTSETFQGLPRLLRLNISGNIFSSLQPGVFDELPALKVVDLGTEFLTCDCH
LRWLLPWAQNRSLQLSEHTLCAYPSALHAQALGSLQEAQLCCEGALELHTHHLIPSLRQVVFQGDRLP
FQCSASYLGNDTRIRWYHNRAPVEGDEQAGILLAESLIHDCTFITSELTLSHIGVWASGEWECTVSMA
QGNASKKVEIVVLETSASYCPAERVANNRGDFRWPRTLAGITAYQSCLQYPFTSVPLGGGAPGTRASR
RCDRAGRWEPGDYSHCLYTNDITRVLYTFVLMPINASNALTLAHQLRVYTAEAASFSDMMDVVYVAQM
IQKFLGYVDQIKELVEVMVDMASNLMLVDEHLLWLAQREDKACSRIVGALERIGGAALSPHAQHISVN
ARNVALEAYLIKPHSYVGLTCTAFQRREGGVPGTRPGSPGQNPPPEPEPPADQQLRFRCTTGRPNVSL
SSFHIKNSVALASIQLPPSLFSSLPAALAPPVPPDCTLQLLVFRNGRLFHSHSNTSRPGAAGPGKRRG
VATPVIFAGTSGCGVGNLTEPVAVSLRHWAEGAEPVAAWWSQEGPGEAGGWTSEGCQLRSSQPNVSAL
HCQHLGNVAVLMELSLE

| | | |
|---|---|---|
| NOV10g, CG50925-03 | SEQ ID NO: 269 | 1329 bp |
| DNA Sequence | ORF Start: at 7 | ORF Stop: at 1324 |

GGATCCCGGCTGCCCTTCCAGTGCTCTGCCAGCTACCTGGGCAACGACACCCGCATCCGCTGGTACCA
CAACCGAGCCCCTGTGGAGGGTGATGAGCAGGCGGGCATCCTCCTGGCCGAGAGCCTCATCCACGACT
GCACCTTCATCACCAGGTGGCCCCGAACTCTGGCTGGCATCACAGCCTACCAGTCCTGCCTGCAGTAT
CCCTTCACCTCAGTGCCCCTGGGCGGGGGTGCCCCGGGCACCCGAGCCTCCCGCCGGTGTGACCGTGC
CGGCCGCTGGGAGCCAGGGGACTACTCCCACTGTCTCTACACCAACGACATCACCAGGGTGCTGTACA
CCTTCGTGCTGATGCCCATCAATGCCTCCAATGCGCTGACCCTGGCTCACCAGCTGCGCGTGTACACA
GCCGAGGCCGCTAGCTTTTCAGACATGATGGATGTGGTCTATGTGGCTCAGATGATCCAGAAATTTTT
GGGTTATGTCGACCAGATCAAAGAGCTGGTAGGGGTGATGGTGGACATGGCCAGCAACCTGATGCTGG
TGGACGAGCACCTGCTGTGGCTGGCCCAGCGCGAGGACAAGGCCTGCAGCCGCATCGTGGGTGCCCTG
GAGCGCATTGGGGGGGCCGCCCTCAGCCCCCATGCCCAGCACATCTCAGTGAATGCGAGGAACGTGGC
ATTGGAGGCCTACCTCATCAAGCCGCACAGCTACGTGGGCCTGACCTGCACAGCCTTCCAGAGGAGGG
AGGGAGGGGTGCCGGGCACACGGCCAGGAAGCCCTGGCCAGAACCCCCCACCTGAGCCCGAGCCCCCA
GCTGACCAGCAGCTCCGCTTCCGCTGCACCACCGGGAGGCCCAATGTTTCTCTGTCGTCCTTCCACAT
CAAGAACAGCGTGGCCCTGGCCTCCATCCAGCTGCCCCCGAGTCTATTCTCATCCCTTCCGGCTGCCC
TGGCTCCCCCGGTGCCCCCAGACTGCACCCTGCAACTGCTCGTCTTCCGAAATGGCCGCCTCTTCCAC
AGCCACAGCAACGCCTCCCGCCCTGGAGCTGCTGGGCCTGGCAAGAGGCGTGGCGTGGCCACCCCCGT
CATCTTCGCAGGAACCAGTGGCTGTGGCGTGGGAAACCTGACAGAGTCAGTGGCCGTTTCGCTGCGGC
ACTGGGCTGAGGGAGCCGAACCTGTGGCCGCTTGGTGGAGCCAGGAGGGGCCCGGGGAGGCTGGGGGC
TGGACCTCGGAGGGCTGCCAGCTCCGCTCCAGCCAGCCCAATGTCAGCGCCCTGCACTGCCAGCACTT
GGGCAATGTGGCCGTGCTCATGGAGCTGAGCCTCGAG

| | | | |
|---|---|---|---|
| NOV10g, CG50925-03 | SEQ ID NO: 270 | 439 aa | MW at 47486.5kD |
| Protein Sequence | | | |

RLPFQCSASYLGNDTRIRWYHNRAPVEGDEQAGILLAESLIHDCTFITRWPRTLAGITAYQSCLQYPF
TSVPLGGGAPGTRASRRCDRAGRWEPGDYSHCLYTNDITRVLYTFVLMPINASNALTLAHQLRVYTAE
AASFSDMMDVVYVAQMIQKFLGYVDQIKELVGVMVDMASNLMLVDEHLLWLAQREDKACSRIVGALER
IGGAALSPHAQHISVNARNVALEAYLIKPHSYVGLTCTAFQRREGGVPGTRPGSPGQNPPPEPEPPAD
QQLRFRCTTGRPNVSLSSFHIKNSVALASIQLPPSLFSSLPAALAPPVPPDCTLQLLVFRNGRLFHSH
SNASRPGAAGPGKRRGVATPVIFAGTSGCGVGNLTESVAVSLRHWAEGAEPVAAWWSQEGPGEAGGWT
SEGCQLRSSQPNVSALHCQHLGNVAVLMELS

| | | |
|---|---|---|
| NOV10h, CG50925-04 | SEQ ID NO: 271 | 60 bp |
| DNA Sequence | ORF Start: at 1 | ORF Stop: end of sequence |

GAGAAGCTTCTCATGAGAGCACATTTTTCTGATGAAGATGAAAAGACGGCCTGTCCTTCT

| | | | |
|---|---|---|---|
| NOV10h, CG50925-04 | SEQ ID NO: 272 | 20 aa | MW at 2307.5kD |
| Protein Sequence | | | |

EKLLMRAHFSDEDEKTACPS

| | | |
|---|---|---|
| NOV10i, CG50925-05 | SEQ ID NO: 273 | 57 bp |
| DNA Sequence | ORF Start: at 1 | ORF Stop: end of sequence |

CGAGCCTCCCGCCGGTGTGACCGTGCCGGCCGCTGGGAGCCAGGGGACTACTCCCAC

| | | | |
|---|---|---|---|
| NOV10i, CG50925-05 | SEQ ID NO: 274 | 19 aa | MW at 2275.4kD |
| Protein Sequence | | | |

RASRRCDRAGRWEPGDYSH

| | | |
|---|---|---|
| NOV10j, CG50925-06 | SEQ ID NO: 275 | 57 bp |
| DNA Sequence | ORF Start: at 1 | ORF Stop: end of sequence |

GAGCTGGTAGAGGTGATGGTGGACATGCCCAGCAACTTGATGCTGGTGGACGAGCAC

| | | | |
|---|---|---|---|
| NOV10j, CG50925-06 | SEQ ID NO: 276 | 19 aa | MW at 2200.6kD |
| Protein Sequence | | | |

ELVEVMVDMPSNLMLVDEH

| | | |
|---|---|---|
| NOV10k, CG50925-07 | SEQ ID NO: 277 | 66 bp |
| DNA Sequence | ORF Start: at 1 | ORF Stop: end of sequence |

AAGGCGGGCAACAGCAGGGCCTCCCTGGAGGCAGGGGAGGAGCTGAGGGGTTCCACCAGGCTCAGG

| | | | |
|---|---|---|---|
| NOV10k, CG50925-07 | SEQ ID NO: 278 | 22 aa | MW at 2358.6kD |
| Protein Sequence | | | |

KAGNSHASLEAGEELRGSTRLR

| | | |
|---|---|---|
| NOV10l, SNP13380040 | SEQ ID NO: 279 | 7892 bp |
| of CG50925-01, | ORF Start: ATG at 1 | ORF Stop: TAA at 4627 |
| DNA Sequence | SNP Pos: 2164 | SNP Change: G to A |

ATGCGGGGGGCGCCCGCGCGCCTGCTGCTGCCGCTGCTGCCGTGGCTCCTGCTGCTCCTGGCGCCCGA
GGCTCGGGGCGCGCCCGGCTGCCCGCTATCCATCCGCAGCTGCAAGTGCTCGGGGGAGCGGCCCAAGG
GGCTGAGCGGCGGCGTCCCTGGCCCGGCTCGGCGGAGGGTGGTGTGCAGCGGCGGGGACCTCCCGGAG
CCTCCCGAGCCCGGCCTTCTGCCTAACGGCACCGTTACCCTTGGCAACCGTTTCTCCACTGCCCCTTG
CCCGCAGTCGGGGCTGGCCCAGTCTTTTCGATTGTGTCCAGAACAATACAGTAACGCCTCTGGTTGGG
TGAGTTCAGAGGCTGCCCGCTTCCTGACAGGACACAACTGGTCAGCCTCTGACGCTGGCCGCCCGGCC
TGCATTTTCCCCGGCCCGGTCTCCTGGAGACAGGTACCAGCTTACAACGCCGGACAGGATCAGGGTAG
CAAATATAGCTTCTATGGAGGAAAGGTCAAAGATGAGTATCTGCGTAGGTCCCCGATCTGTCGCTCTG
CTGCCGCCCTGCACACTGACACCAGTGCCCGCAGCTGGTCTGAATTTAGGCTCTTGAGCAATAACAAG
ATCACGGGGCTCCGCAATGGCTCCTTCCTGGGACTGTCACTGCTGGAGAAGCTTCTCATGAGAGCACA
TTTTTCTGATGAAGATGAAAAGACGGCCTGTCCTTCTCCAGCACCAAGGCTCTGGAGGCTGCATAGCA
AGTGCACCCTGAGCATGGCAGGCAATCAGAAGAACCAGGGCGGCAGCAGCTCACAAGAGGCAGCATCT
GGCTTGATGCCGAGTCGGCGGCTCCAGCGGGGAGAGGTTGAGGGCCTCTGCACTTGCCTCTGCACGGA
CCTGAGGAACAACATCATCAGCACAGTGCAGCCGGGCGCCTTCCTGGGCCTGGGGGAGCTGAAGCGTT
TAGATCTCTCCAACAACCGGATTGGCTGTCTCACCTCCGAGACCTTCCAGGGCCTCCCCAGGCTTCTC
CGACTAAACATATCTGGAAACATCTTCTCCAGTCTGCAACCTGGGGTCTTTGATGAGCTGCCAGCCCT
TAAGGTTGTGGACTTGGGCACCGAGTTCCTGACCTGTGACTGCCACCTGCGCTGGCTGCTGCCCTGGG
CCCAGAATCGCTCCCTGCAGCTGTCGGAACACACGCTCTGTGCTTACCCCAGTGCCCTGCATGCTCAT
GCCCTGGGCAGCCTCCAGGAGGCCCAGCTCTGCTGCGAGGGGGCCCTGGAGCTGCACACACACCACCT
CATCCCGTCCCTACGCCAAGTGGTGTTCCAGGGGGATCGGCTGCCCTTCCAGTGCTCTGCCAGCTACC
TGGGCAACGACACCCGCATCCGCTGGTACCACAACCGAGCCCCTGTGGAGGGTGATGAGCAGGCGGGC
ATCCTCCTGGCCGAGAGCCTCATCCACGACTGCACCTTCATCACCAGTGAGCTGACGCTGTCTCACAT
CGGCGTGTGGGCCTCAGGCGAGTGGGAGTGCACCGTGTCCATGGCCCAAGGCAACGCCAGCAAGAAGG
TGGAGATCGTGGTGCTGGAGACCTCTGCCTCCTACTGCCCCGCCGAGCGTGTTGCCAACAACCGCGGG
GACTTCAGGTGGCCCCGAACTCTGGCTGGCATCACAGCCTACCAGTCCTGCCTGCAGTATCCCTTCAC
CTCAGTGCCCCTGGGCGGGGGTGCCCCGGGCACCCGAGCCTCCCGCCGGTGTGACCGTGCCGGCCGCT
GGGAGCCAGGGGACTACTCCCACTGTCTCTACACCAACGACATCACCAGGGTGCTGTACACCTTCGTG
CTGATGCCCATCAATGCCTCCAATGCGCTGACCCTGGCTCACCAGCTGCGCGTGTACACAGCCGAGGC
CGCTAGCTTTTCAGACATGATGGATGTAGTCTATGTGGCTCAGATGATCCAGAAATTTTTGGGTTATG
TCGACCAGATCAAAGAGCTGGTAGAGGTGATGGTGGACATGCCCAGCAACTTGATGCTGGTGGACGAG
CACCTGCTGTGGCTGGCCCAGCGCGAGGACAAGGCCTGCAGCCGCATCGTGGGTGCCATAGAGCGCAT
TGGGGGGGCCGCCCTCAGCCCCCATGCCCAGCACATCTCAGTGAATGCGAGGAACATGGCATTGGAGG
CCTACCTCATCAAGCCGCACAGCTACGTGGGCCTGACCTGCACAGCCTTCCAGAGGAGGGAGGGAGGG
GTGCCGGGCACACGGCCAGGAAGCCCTGGCCAGAACCCCCCACCTGAGCCCGAGCCCCCAGCTGACCA
GCAGCTCCGCTTCCGCTGCACCACCGGGAGGCCCAATGTTTCTCTGTCGTCCTTCCACATCAAGAACA
CCGGTGCCCCCAGACTGCACCCTGCAACTGCTCGTCTTCCGAAATGGCCGCCTCTTCCACAGCCACAG
CAACACCTCCCGCCCTGGAGCTGCTGGGCCTGGCAAGAGGCGTGGCGTGGCCACCCCCGTCATCTTCG
CAGGAACCAGTGGCTGTGGCGTGGGAAACCTGACAGAGCCAGTGGCCGTTTCGCTGCGGCACTGGGCT
GAGGGAGCCGAACCTGTGGCCGCTTGGTGGAGCCAGGAGGGGCCCGGGGAGGCTGGGGGCTGGACCTC
GGAGGGCTGCCAGCTCCGCTCCAGCCAGCCCAATGTCAGCGCCCTGCACTGCCAGCACTTGGGCAATG
TGGCCGTGCTCATGGAGCTGAGCGCCTTTCCCAGGGAGGTGGGGGGCGCCGGGGCAGGGCTGCACCCC
GTGGTATACCCCTGCACGGCCTTGCTGCTGCTCTGCCTCTTCGCCACCATCATCACCTACATCCTCAA
CCACAGCTCCATCCGTGTGTCCCGGAAAGGCTGGCACATGCTGCTGAACTTGTGCTTCCACATAGCCA
TGACCTCTGCTGTCTTTGCGGGGGGCATCACACTCACCAACTACCAGATGGTCTGCCAGGCGGTGGGC
ATCACCCTGCACTACTCCTCCCTATCCACGCTGCTCTGGATGGGCGTGAAGGCGCGAGTGCTCCATAA
GGAGCTCACCTGGAGGGCACCCCCTCCGCAAGAAGGGGACCCCGCTCTGCCTACTCCCAGTCCTATGC
TCCGGTACATACTTTCAATTCCAGCTTTGCAATTGGGGAGGGACTCCAACGCAGGCGTAGGAAACCTC
CCAAGGTTCTATTTGATCGCTGGAGGGATTCCACTCATTATCTGTGGCATCACAGCTGCAGTCAACAT
CCACAACTACCGGGACCACAGCCCCTACTGCTGGCTGGTGTGGCGTCCAAGCCTTGGCGCCTTCTACA
TCCCTGTGGCTTTGATTCTGCTCATCACCTGGATCTATTTCCTGTGCGCCGGGCTACGCTTACGGGGT
CCTCTGGCACAGAACCCCAAGGCGGGCAACAGCAGGGCCTCCCTGGAGGCAGGGGAGGAGCTGAGGGG
TTCCACCAGGCTCAGGGGCAGCGGCCCCCTCCTGAGTGACTCAGGTTCCCTTCTTGCTACTGGGAGCG
CGCGAGTGGGGACGCCCGGGCCCCCGGAGGATGGTGACAGCCTCTATTCTCCGGGAGTCCAGCTAGGG
GCGCTGGTGACCACGCACTTCCTGTACTTGGCCATGTGGGCCTGCGGGGCTCTGGCAGTGTCCCAGCG
CTGGCTGCCCCGGGTGGTGTGCAGCTGCTTGTACGGGGTGGCAGCCTCCGCCCTGGGCCTCTTCGTCT
TCACTCACCACTGTGCCAGGCGGAGGGACGTGAGAGCCTCGTGGCGCGCCTGCTGCCCCCCTGCCTCT
CCCGCGGCCCCCCATGCCCCGCCCCGGGCCCTGCCCGCCGCCGCAGAGGACGGTTCCCCGGTGTTCGG
GGAGGGGCCCCCCTCCCTCAAGTCCTCCCCAAGCGGCAGCAGCGGCCATCCGCTGGCTCTGGGCCCCT
GCAAGCTCACCAACCTGCAGCTGGCCCAGAGTCAGGTGTGCGAGGCGGGGGCGGCGGCCGGCGGGGAA
GGAGAGCCGGAGCCGGCGGGCACCCGGGGAAACCTCGCCCACCGCCACCCCAACAACGTGCACCACGG
GCGTCGGGCGCACAAGAGCCGGGCCAAGGGACACCGCGCGGGGGAGGCCTGCGGCAAGAACCGGCTCA
AGGCCCTGCGCGGGGGCGCGGCGGGGGCGCTGGAACTGCTGTCCAGCGAGAGCGGTAGTCTGCACAAC
AGCCCCACCGACAGCTACCTGGGCAGCAGCCGCAACAGCCCGGGCGCCGGCCTGCAGCTGGAAGGCGA
GCCCATGCTCACGCCGTCCGAGGGCAGCGACACCAGCGCCGCGCCGCTTTCTGAGGCGGGCCGGGCAG
GCCAGCGCCGCAGCGCCAGCCGCGACAGTCTCAAGGGCGGCGGCGCGCTGGAGAAGGAGAGCCATCGC
CGCTCGTACCCGCTCAACGCCGCCAGCCTAAACGGCGCCCCCAAGGGGGGCAAGTACGACGACGTCAC
CCTGATGGGCGCGGAGGTAGCCAGCGGCGGCTGCATGAAGACCGGACTCTGGAAGAGCGAAACTACCG
TCTAAGGTGGGGCGGGCGACGCGGTAGACGGGTTGGCCACGCGGCTCGTTCCCCCGCTCCTCGGGGCC
CTCCAAGGTGTCTCCGTAGTCAGCAGGTTGGAGGCAGAGGAGCCGATGGCTGGAGGAAGCCCACAGGC
GGATGTTCCCCACTTGCCTAGAGGGCATCCCTCTGGGGTAGCGACAGACAATCCCAGAAACACGCATA
ATACATTTCCGTCCAGCCCGGGGCAGTCTGACTGTCGGTGCCCTCCCAGGAACGGGGAAGGCCTCCGT
CTGTGTGAAAGGGCACAGCACATCCCAGGTGCACCCTCCCCAAGTACTCCCACCCCGCCTACTGTCCA
TGCGGCCTCACTGGGGGCCATCAGCCTCACCAGCAAAGCAGAGATGAGAGCGTGGGAACTGTGTTCTT
TCCTCCCTGCCCTCTACTGATTTCAGCCCAGCCCCTGCCTAGATCCTAGGTCCCTTTTCCTCCCGAGT
TTGGCTGGCACGAGAGCTAGCCCAGCACATGAAGCAGGTGATGTTAAGTCACAAGGTGCTGCTTTTCA
GATCCACTATGCAAGAGGGGAGGGTGGGGCCACGTGAAAGGCAGCTCTAGACATCAACCAGTCCTGGG
GGAGGGGAGTGGGAACCGGGCACAACTAGGAACAATGCCACCATTCCCACAGGAGTGGTACTTAAACC
AGACAGCAGGGTTCAGAGGTGGCACACCGGGACAAAGCTGAGGCCCTGCACCTCAACAGCTGACTGCC
AGGTGCCTGTGGGTGAACTGAGGGGAGTAGAGGGAGAGGGCAGGTGGAACTGGGGCAGAATCTAGTCA
TGCCCTAAAGCTAGTCCTGTAAACAATGGTGCCCCAGAAAGCTGCAGGTGGTGTTTGGAGAAGCAGTT
ACTTTTCAGTTACAAGACCCATCTCCCTAGTCTCAGCCTTACAACACCACGGGACTAAGGAAGAGCAC
TTCCTTGCCTCCGTAAGGCCAGAGGAAGAACCATCCCAATCATTTGATCTCCAGCTCCACAGTAGAGA
GAAACCTACAAAATGTCAAACCAGCTTCCCGACTCCCAGGAGCTCAAGCCAAGCCCAGAGGCAGTGGC
TGGGGTCCCTGCAGGTCATGAGGGGCCTATGCCTTTACTCCTTTTAAACACCAGCACCCGTCTTTTCC
CCAACCTAAAACCAACCACCAGCATTTCACTACAGGACCAAATGGAAACCGAGGGAACCCTGGGTCTT
GGGAAGAACAACAGGAAACCAAGGTCTGACCTAGGGTTCCCTCCCAGTCTTCACATCACTCTGGCCTC
ATCACCAAGGTGACAGAGGACACAGGGGAGGGGGAAAACCCACACACACTCCTTGGAATGGGTCCTGT
TATTTATGCTTGCTGCACAGACATATTAGAAGAAAAAAAAAAAGCTTTGTATTATTCTTCCACATATG
CTGGCTGCTGTTTACACACCCTGCCAATGCCTTAGCACTGGAGAGCTTTTTGCAATATGCTGGGGAAA
GGGGAGGGAGGGAATGAAAGTGCCAAAGAAAACATGTTTTTAAGAACTCGGGTTTTATACAATAGAAT
GTTTTCTAGCAGATGCCTCTTGTTTTAATATATTAAAATTTTGCAAAGCCCTTTGAGCTACTGCCTTA
GTCTACCCACTGTCCTTTTGTTATGAGGTAGAGGATCTCATGACACCATACACACAAACCCATCATTG
CCTGTGAATGCACGTAGGGCCAGAATTCCCCAGTTCCCGCTCCTCTGAGGGTTGATACTGCTGGGAAT
GCCAACCACTCCACAAGCAGAGGGAAGCCCCCTCAGGCCTGCAGGAGGAGCCGCAGCAGTGTGTCCAA
TTCAAACCAGCAGCAAAGAGCCTGACATTTTCCCATCCATCTATGAGGAAAGCCATCTCACAGAACAT
GGACATAGGCAACTTGCTCTCCCACACCAAGGGATGGGAATCTCTCCTACCTATAGTCATCCCTGCAC
TCCTGACTTTACTCCAGGACCCAGGGTCCAACTAATGGCAGAGCCCCTCTTGGTTCCTTCAAACAAGA
AAAGCAATACCTACGGACTGGTGTACACTTCCATCCTTGGTTATAACAGGAATGTTATCAAGCTGTCA
GAACAGGATGAAGTGCTCCCAGTGGATATCCATCAGGGAGGGTTAGGGACACTCGTGGCAGCCTGTCT
AGCAGCCTGGGCTCTCTGAAAGTCCCTAACTTCCTGAGGGGTACGCAAATACTGTTCTATTTCACTAT
CAGAAATGTTCTCATCTCCAGTGACAGTGGAGACAGGGGGTACAGGGCAGATCCGCTTCGGGGACTTC
AACATGCAGGGTGGCAAGAGAAGGGCAGGACTGGCCGGCCGCTTCCCCTGGGGTAAACCTAAGGAATT
ATTTCCCACCTCCCCTTCTCCTTGCCCCTGTCCCCACCCCGGTGGCTCCTTCTCTCGGGTCTCCACTT
CTGCTGTCCCATCCCGAAAGGCAGAGCGGACCAGTGACTGGCGGTGCTGGAGAAGGTCACCGATGTGC
TTCACCACAGACCGTTTGTCAAGTCTCAGAACTCGTAACCAGGCCAGCTGCTCAGCCATCCGCAGCAG
CACAGCCAGCAGCTCCTGCAGGCGGGAGGACGCCGGGTAGGGCAGGTCCACATTTGCCAATTTACAAA
ATCGGGCAAGGGAACATGAAAGCCGATCTGCAGGCTGCAGCGACTGCCAAGCCAGGAAAGTCGCAGCA
GTGATGACGGGCAAGGGATGCCTCCCGGTCACCAGCCACGTCTCATTTGCCAGCTCCACCAACTGCAT
TGTTCGAGACAGCATCTTCTCTTTGTCTTCCACGTATTTGGCTGGCACAGAAGGTGAAGCTTGGAACA
GTTTGAAGCTGAAATAACCAAAATGAGGGTTGGATCTTAATGATATAGGGGCTGCTCTCCCACAGTGA
GGAAAGACAGCCCACTCAAGATGGGGAAGCTATTCTGCCCTCAGGAATACTCAAGCTCACTGGGCAGC
AAGTTAATAAAGGTAGTGAGAGAAAACAGGGCGTCTTCCGCTTGTTAGGGGAAGGTGGAGGGATGGAG
GAGAGCACGAACATTTATTGGGCGCCTCCCAATCACCATTATTCTGAGTGCTTTACAACGTTCTCATT
TAATCTACGTGCACGTGCACCATCTTATGTGCATGTATAGTTAAAAAACTTTCCCATAGTCATCCAGC
CAGGCAGTAACCAAGCTTCAAATACAAGGCTATTTGACACCAACAGCCTCTACTTTCAACGTTATTTA
TCAG

| | | | |
|---|---|---|---|
| NOV10l, SNP13380040 | SEQ ID NO: 280 | 1542 aa | MW at 164933.2kD |
| of CG50925-01, | SNP Pos: 722 | SNP Change: Val to Met | |
| Protein Sequence | | | |

MRGAPARLLLPLLPWLLLLLAPEARGAPGCPLSIRSCKCSGERPKGLSGGVPGPARRRVVCSGGDLPE
PPEPGLLPNGTVTLGNRFSTAPCPQSGLAQSFRLCPEQYSNASGWVSSEAARFLTGHNWSASDAGRPA
CIFPGPVSWRQVPAYNAGQDQGSKYSFYGGKVKDEYLRRSPICRSAAALHTDTSARSWSEFRLLSNNK
ITGLRNGSFLGLSLLEKLLMRAHFSDEDEKTACPSPAPRLWRLHSKCTLSMAGNQKNQGGSSSQEAAS
GLMPSRRLQRGEVEGLCTCLCTDLRNNIISTVQPGAFLGLGELKRLDLSNNRIGCLTSETFQGLPRLL
RLNISGNIFSSLQPGVFDELPALKVVDLGTEFLTCDCHLRWLLPWAQNRSLQLSEHTLCAYPSALHAH
ALGSLQEAQLCCEGALELHTHHLIPSLRQVVFQGDRLPFQCSASYLGNDTRIRWYHNRAPVEGDEQAG
ILLAESLIHDCTFITSELTLSHIGVWASGEWECTVSMAQGNASKKVEIVVLETSASYCPAERVANNRG
DFRWPRTLAGITAYQSCLQYPFTSVPLGGGAPGTRASRRCDRAGRWEPGDYSHCLYTNDITRVLYTFV
LMPINASNALTLAHQLRVYTAEAASFSDMMDVVYVAQMIQKFLGYVDQIKELVEVMVDMPSNLMLVDE
HLLWLAQREDKACSRIVGAIERIGGAALSPHAQHISVNARNMALEAYLIKPHSYVGLTCTAFQRREGG
VPGTRPGSPGQNPPPEPEPPADQQLRFRCTTGRPNVSLSSFHIKNSVALASIQLPPSLFSSLPAALAP
PVPPDCTLQLLVFRNGRLFHSHSNTSRPGAAGPGKRRGVATPVIFAGTSGCGVGNLTEPVAVSLRHWA
EGAEPVAAWWSQEGPGEAGGWTSEGCQLRSSQPNVSALHCQHLGNVAVLMELSAFPREVGGAGAGLHP
VVYPCTALLLLCLFATIITYILNHSSIRVSRKGWHMLLNLCFHIAMTSAVFAGGITLTNYQMVCQAVG
ITLHYSSLSTLLWMGVKARVLHKELTWRAPPPQEGDPALPTPSPMLRYILSIPALQLGRDSNAGVGNL
PRFYLIAGGIPLIICGITAAVNIHNYRDHSPYCWLVWRPSLGAFYIPVALILLITWIYFLCAGLRLRG
PLAQNPKAGNSRASLEAGEELRGSTRLRGSGPLLSDSGSLLATGSARVGTPGPPEDGDSLYSPGVQLG
ALVTTHFLYLAMWACGALAVSQRWLPRVVCSCLYGVAASALGLFVFTHHCARRRDVRASWRACCPPAS
PAAPHAPPRALPAAAEDGSPVFGEGPPSLKSSPSGSSGHPLALGPCKLTNLQLAQSQVCEAGAAAGGE
GEPEPAGTRGNLAHRHPNNVHHGRRAHKSPAKGHRAGEACGKNRLKALRGGAAGALELLSSESGSLHN
SPTDSYLGSSRNSPGAGLQLEGEPMLTPSEGSDTSAAPLSEAGRAGQRRSASRDSLKGGGALEKESHR
RSYPLNAASLNGAPKGGKYDDVTLMGAEVASGGCMKTGLWKSETTV

A ClustalW comparison of the above protein sequences yields the following sequence alignment shown in Table 10B.

TABLE 10B
Comparison of the NOV10 protein sequences.

| | |
|---|---|
| NOV10a | ------------------------------------------------------------ |
| NOV10b | MRGAPARLLLPLLPWLLLLLAPEARGAPGCPLSIRSCKCSGERPKGLSGGVPGPARRRVV |
| NOV10c | ------------------------------------------------------------ |
| NOV10d | ------------------------------------------------------------ |
| NOV10e | ------------------------------------------------------------ |
| NOV10f | ------------------------------------------------------------ |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | ------------------------------------------------------------ |
| NOV10b | CSGGDLPEPPEPGLLPNGTVTLDLRNNIISTVQPGAFLGLGELKRLDLSNNRIGCLTSET |
| NOV10c | ------------------------------------------------------------ |
| NOV10d | ------------------------------------------------------------ |
| NOV10e | ------------------------------------------------------------ |
| NOV10f | ------------------------------------------------------------ |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | ------------------------------------------------------------ |
| NOV10b | FQGLPRLLRLNISGNIFSSLQPGVFDELPALKVVDLGTEFLTCDCHLRWLLPWAQNRSLQ |
| NOV10c | ------------------------------------------------------------ |
| NOV10d | ------------------------------------------------------------ |
| NOV10e | ------------------------------------------------------------ |
| NOV10f | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | ------------------------------------------------------------ |
| NOV10b | LSEHTLCAYPSALHAHALGSLQEAQLCCEGALELHTHHLIPSLRQVVFQGDRLPFQCSAS |
| NOV10c | ------------------------------------------------------------ |
| NOV10d | ------------------------------------------------------------ |
| NOV10e | ------------------------------------------------------------ |
| NOV10f | ------------------------------------------------------------ |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | ------------------------------------------------------------ |
| NOV10b | YLGNDTRIRWYHNRAPVEGDEQAGILLAESLIHDCTFITSELTLSHIGVWASGEWECTVS |
| NOV10c | ------------------------------------------------------------ |
| NOV10d | ------------------------------------------------------------ |
| NOV10e | ------------------------------------------------------------ |
| NOV10f | ------------------------------------------------------------ |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | ------------------------------------------------------------ |
| NOV10b | MAQGNASKKVEIVVLETSASYCPAERVANNRGDFRWPRTLAGITAYQSCLQYPFTSVPLG |
| NOV10c | ------------------------------------------------------------ |
| NOV10d | ------------------------------------------------------------ |
| NOV10e | ------------------------------------------------------------ |
| NOV10f | ------------------------------------------------------------ |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | ------------------------------------------------------------ |
| NOV10b | GGAPGTRASRRCDRAGRWESGDYSHCLYTNDITRVLYTFVLMPINASNALTLAHQLRVYT |
| NOV10c | ------------------------------------------------------------ |
| NOV10d | ------------------------------------------------------------ |
| NOV10e | ------------------------------------------------------------ |
| NOV10f | ------------------------------------------------------------ |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | ------------------------------------------------------------ |
| NOV10b | AEAASFSDMMDVVYVAQMIQKFLGYVDQIKELVEVMVDMASNLNLVDEHLLWLAQREDKA |
| NOV10c | ------------------------------------------------------------ |
| NOV10d | ------------------------------------------------------------ |
| NOV10e | ------------------------------------------------------------ |
| NOV10f | ------------------------------------------------------------ |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | ------------------------------------------------------------ |
| NOV10b | CSRIVGALERIGGAALSPHAQHISVNARNVALEAYLIKPHSYVGLTCTAFQRREGGVPGT |
| NOV10c | ------------------------------------------------------------ |
| NOV10d | ------------------------------------------------------------ |
| NOV10e | ------------------------------------------------------------ |
| NOV10f | ------------------------------------------------------------ |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | ------------------------------------------------------------ |
| NOV10b | RPGSPGQNPPPEPEPPADQQLRFRCTTGRPNVSLSSFHIKNSVALASIQLPPSLFSSLPA |
| NOV10c | ------------------------------------------------------------ |
| NOV10d | --------------------------------------------TGSAPGCPLSIRSCKC |
| NOV10e | ------------------------------------------MRGAPARLLLPLLPWLLL |
| NOV10f | ------------------------------------------------------------ |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | ------------------------------------------------------------ |
| NOV10b | ALAPPVPPDCTLQLLVFRNGRLFHSHSNTSRPGAAGPGKRRGVATPVIFAGTSGCGVGNL |
| NOV10c | ------------------------------------------------------------ |
| NOV10d | SGERAKGLSGGVPGPARRRVVCSGGDLPEPPEPGLLPNGTVTLLLSNNKITGLRNGSFLG |
| NOV10e | LLAPEARGAPGCPLSIRSCKCSGERPKGLSGGVPGPARRRVVCSGGDLPEPPEPGLLPNG |
| NOV10f | ------------------------------------------------------------ |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | ------------------------------------------------------------ |
| NOV10b | TEPVAVSLRHWAEGAEPVAAWWSQEGPGEAGGWTSEGCQLRSSQPNVSALHCQHLGNVAV |
| NOV10c | ------------------------------------------------------------ |
| NOV10d | LSLLEKLDLRNNIISTVQPGAFLGLGELKRLDLSNNRIGCLTSETFQGLPRLLRLNISGN |
| NOV10e | TVTLGNRFSTAPCPQSGLAQSFRLCPEQYSNASGWVSSEAARFLTGHNWSASDAGRPACI |
| NOV10f | ------------------------------------------------------------ |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | -----------MRGAPARLLLPLLPWLLLLLAPEARGAPGCPLSIRSCKCSGERPKGLSG |
| NOV10b | LMELSAFPREVGGAGAGLHPVVYPCTALLLLCLFATIITYILNNSSIRVSRKGWHMLLNL |
| NOV10c | ------------------------------------------------------------ |
| NOV10d | IFSSLQPGVFDELPALKVVDLGTEFLTCDCHLRWLLPWAQNRSLQLSEHTLCAYPSALHA |
| NOV10e | FPGPVSWRQVPAYNAGQDQGSKYSFYGGKVKDEYLRRSPICRSAAALHTDTSARSWSEFR |
| NOV10f | ------------------------------------------------------------ |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | GVPGPARRRVVCSGGDLPEPPEPGLLPNGTVTLGNRFSTAPCPQSGLAQSFRLCPEQYSN |
| NOV10b | CFHIAMTSAVFAGGITLTNYQMVCQAVGITLHYSSLSTLLWMGVKARVLHKELTWRAPPP |
| NOV10c | ------------------------------------------------------------ |
| NOV10d | QALGSLQEAQLCCEGALELHTHHLIPSLRQVVFQGDRLPFQCSASYLGNDTRIRWYHDEA |
| NOV10e | LLSNNKITGLRNGSFLGLSLLEKLLMRAHFSDEDEKTACPSPAPRLWRLHSKCTLSMAGN |
| NOV10f | ------------------------------------------------------------ |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | ASGWVSSEAARFLTGHNWSASDAGRPACIFPGPVSWRQVPAYNAGQDQGSKYSFYGGKVK |
| NOV10b | QEGDPALPTPSPMLRYILSIPALQLGRDSNAGVGNLPRFYLIAGGIPLIICGITAAVNIH |
| NOV10c | ----------------------------------------------------TGSELKRL |
| NOV10d | PVEGDEQAGILLAESLIHDCTFITSELTLSHIGVWASGEWECTVSMAQGNASKKVEIVVL |
| NOV10e | QKNQGGSSSQEAASGLMPSRRLQRGEVEGLCTCLCTDLRNNIISTVQPGAFLGLGELKRL |
| NOV10f | ----------------------------------------------------TGSELKRL |
| NOV10g | -------------------------------RLPFQCSASYLGNDTRIRWYHNHAPVEGD |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | DEYLRRSPICRSAAALHTDTSARSWSEFRLLSNNKITGLRNGSFLGLSLLEKLLMRAHFS |
| NOV10b | NYRDHSPYCWLVWRPSLGAFYIPVALILLITWIYFLCAGLRLRGPLAQNPKAGNSRASLE |
| NOV10c | DLSNNRIGCLTSETFQGLPRLLRLNISGNIFSSLQPGVFDELPALKVVDLGTEFLTCDCH |
| NOV10d | ETSASYCPAERVANNRGDFRWPRTLAGITAYQSCLQYPFTSVPLGGGAPGTRASRRCDRA |
| NOV10e | DLSNNRIGCLTSETFQGLPRLLRLNISGNIFSSLQPGVFDELPALKVVDLGTEFLTCDCH |
| NOV10f | DLSNNRIGCLTSETFQGLPRLLRLNISGNIFSSLQPGVFDELPALKVVDLGTEFLTCDCH |
| NOV10g | EQAGILLAESLIHDCTFITRWPRTLAGITAYQSCLQYPFTSVPLGGGAPGTHASRRCDRA |
| NOV10h | --------------------------------------------------EKLLMRAHFS |
| NOV10i | ---------------------------------------------------RASRRCDRA |
| NOV10j | --------------------------------------------------ELVEVMVDMP |
| NOV10k | --------------------------------------------------KAGNSRASLE |
| NOV10a | DEDEKTACPSPAPRLWRLHSKCTLSMAGNQKNQGGSSSQEAASGLMPSRRLQRGEVEGLC |
| NOV10b | AGEELRGSTRLRGSGPLLSDSGSLLATGSARVGTPGPPEDGDSLYSPGVQLGALVTTHFL |
| NOV10c | LRWLLPWAQNRSLQLSEHTLCAYPSALHAQALGSLQEAQLCCELE--------------- |
| NOV10d | GRWEPGDYSHCLYTNDITRVLYTFVLMPINASNALTLAHQLRVYTAEAASFSDNMDVVYV |
| NOV10e | LRWLLPWAQNRSLQLSEHTLCAYPSALHAHALGSLQEAQLCCEGALELHTHHLIPSLRQV |
| NOV10f | LRWLLPWAQNRSLQLSEHTLCAYPSALHAQALGSLQEAQLCCEGALELHTHHLIPSLRQV |
| NOV10g | GRWEPGDYSHCLYTNDITRVLYTFVLMPINASNALTLAHQLRVYTAEAASFSDMMDVVYV |
| NOV10h | DEDEKTACPS-------------------------------------------------- |
| NOV10i | GRWEPGDYSH-------------------------------------------------- |
| NOV10j | SNLMLVDEH--------------------------------------------------- |
| NOV10k | AGEELRGSTRLR------------------------------------------------ |
| NOV10a | TCLCTDLRNNIISTVQPGAFLGLGELKRLDLSNNRIGCLTSETFQGLPRLLRLNISGNIF |
| NOV10b | YLAMWACGALAVSQRWLPRVVCSCLYGVAASALGLFVFTHHCARPPDVRASWRACCPPAS |
| NOV10c | ------------------------------------------------------------ |
| NOV10d | AQMIQKFLGYVDQIKELVEVMVDMASNLMLVDEHLLWLAQREDKACSRIVGALERIGGAA |
| NOV10e | VFQGDRLPFQCSASYLGNDTRIRWYHNRAPVEGDEQAGILLAESLIHDCTFITSELTLSH |
| NOV10f | VFQGDRLPFQCSASYLGNDTRIRWYHNRAPVEGDEQAGILLAESLIHDCTFITSELTLSH |
| NOV10g | AQMIQKFLGYVDQIKELVGVMVDMASNLMLVDEHLLWLAQREDKACSRIVGALERIGGAA |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | SSLQPGVFDELPALKVVDLGTEFLTCDCHLRWLLPWAQNRSLQLSEHTLCAYPSALHAHA |
| NOV10b | PAAPHAPPRALPAAAEDGSPVFGEGPPSLKSSPSGSSGHPLALGPCKLTNLQLAQSQVCE |
| NOV10c | ------------------------------------------------------------ |
| NOV10d | LSPHAQHISVNARNVALEAYLIKPHSYVGLTCTAFQRREGGVPGTRPGSPGQNPPPEPEP |
| NOV10e | IGVWASGEWECTVSMAQGNASKKVEIVVLETSASYCPAERVANNRGDFRWPRTLAGITAY |
| NOV10f | IGVWASGEWECTVSMAQGNASKKVEIVVLETSASYCPAERVANNRGDFRWPRTLAGITAY |
| NOV10g | LSPHAQHISVNARNVALEAYLIKPHSYVGLTCTAFQRREGGVPGTRPGSPGQNPPPEPEP |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | LGSLQEAQLCCEGALELHTHHLIPSLRQVVFQGDRLPFQCSASYLGNDTRIRWYHNEAPV |
| NOV10b | AGAAAGGEGEPEPAGTRGNLAHRHPNNVHHGRRAHKSRAKGHRAGEACGKNRLKALRGGA |
| NOV10c | ------------------------------------------------------------ |
| NOV10d | PADQQLRFRCTTGRPNVSLSSFHIKNSVALASIQLPPSLFSSLPAALAPPVPPDCTLQLL |
| NOV10e | QSCLQYPFTSVPLGGGAPGTRASRRCDEAGRWEPGDYSHCLYTNDITRVLYTFVLMPINA |
| NOV10f | QSCLQYPFTSVPLGGGAPGTRASRRCDRAGRWEPGDYSHCLYTNDITRVLYTFVLMPINA |
| NOV10g | PADQQLRFRCTTGRPNVSLSSFHIKNSVALASIQLPPSLFSSLPAALAPPVPPDCTLQLL |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | EGDEQAGILLAESLIHDCTFITSELTLSHIGVWASGEWECTVSMAQGNASKKVEIVVLET |
| NOV10b | AGALELLSSESGSLHNSPTDSYLGSSRNSPGAGLQLEGEPMLTPSEGSDTSAAPLSEAGR |
| NOV10c | ------------------------------------------------------------ |
| NOV10d | VFRTGRLFHSHSNTSRPGAAGPGKRRGVATPVIFAGTSGCGVGNLTEPVAVSLRHWAEGA |
| NOV10e | SNALTLAHQLRVYTAEAASFSDMMDVVYVAQMIQKFLGYVDQIKELVEVMVDMPSNLMLV |
| NOV10f | SNALTLAHQLRVYTAEAASFSDMMDVVYVAQMIQKFLGYVDQIKELVEVMVDMASNLMLV |
| NOV10g | VFRNGRLFHSHSNASRPGAAGPGKRRGVATPVIFAGTSGCGVGNLTESVAVSLRHWAEGA |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | SASYCPAERVANNRGDFRWPRTLAGITAYQSCLQYPFTSVPLGGGAPGTRASRRcDRAGR |
| NOV10b | AGQRRSASRDSLKGGGALEKESHRRSYPLNAASLNGAPKGGKYDDVTLMGAEVASGGCMK |
| NOV10c | ------------------------------------------------------------ |
| NOV10d | EPVAAWWSQEGPGEAGGWTSEGCQLRSSQPNVSALHCQHLGNVAVLMELSAFPREVGGAG |
| NOV10e | DEHLLWLAQREDKACSRIVGAIERIGGAALSPHAQHISVNARNVALEAYLIKPHSYVGLT |
| NOV10f | DEHLLWLAQREDKACSRIVGALERIGGAALSPHAQHISVNARNVALEAYLIKPHSYVGLT |
| NOV10g | EPVAAWWSQEGPGEAGGWTSEGCQLRSSQPNVSALHCQHLGNVAVLMELS---------- |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | WEPGDYSHCLYTNDITRVLYTFVLMPINASNALTLAHQLRVYTAEAASFSDMMDVVYVAQ |
| NOV10b | TGLWKSETTV-------------------------------------------------- |
| NOV10c | ------------------------------------------------------------ |
| NOV10d | AGLLEG------------------------------------------------------ |
| NOV10e | CTAFQRREGGVPGTRPGSPGQNPPPEPEPPADQQLRFRCTTGRPNVSLSSFHIKNSVALA |
| NOV10f | CTAFQRREGGVPGTRPGSPGQNPPPEPEPPADQQLRFRCTTGRPNVSLSSFHIKNSVALA |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | MIQKFLGYVDQIKELVEVMVDMPSNLMLVDEHLLWLAQREDKACSRIVGAIERIGGAALS |
| NOV10b | ------------------------------------------------------------ |
| NOV10c | ------------------------------------------------------------ |
| NOV10d | ------------------------------------------------------------ |
| NOV10e | SIQLPPSLFSSLPAALAPPVPPDCTLQLLVFRNGRLFHSHSNTSRPGAAGPGKRRGVATP |
| NOV10f | SIQLPPSLFSSLPAALAPPVPPDCTLQLLVFRNGRLFHSHSNTSRPGAAGPGKRRGVATP |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | PHAQHISVNARNVALEAYLIKPHSYVGLTCTAFQRREGGVPGTRPGSPGQNPPPEPEPPA |
| NOV10b | ------------------------------------------------------------ |
| NOV10c | ------------------------------------------------------------ |
| NOV10d | ------------------------------------------------------------ |
| NOV10e | VIFAGTSGCGVGNLTEPVAVSLRHWAEGAEPVAAWWSQEGPGEAGGWTSEGCQLRSSQPN |
| NOV10f | VIFAGTSGCGVGNLTEPVAVSLRHWAEGAEPVAAWWSQEGPGEAGGWTSEGCQLRSSQPN |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | DQQLRFRCTTGRPNVSLSSFHIKNSVALASIQLPPSLFSSLPAALAPPVPPDCTLQLLVF |
| NOV10b | ------------------------------------------------------------ |
| NOV10c | ------------------------------------------------------------ |
| NOV10d | ------------------------------------------------------------ |
| NOV10e | VSALHCQHLGNVAVLMELSAFPREVGGAGAGLHPVVYPCTALLLLCLFATIITYILNHSS |
| NOV10f | VSALHCQHLGNVAVLMELSLE--------------------------------------- |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | RNGRLFHSHSNTSRPGAAGPGKRRGVATPVIFAGTSGCGVGNLTEPVAVSLRHWAEGAEP |
| NOV10b | ------------------------------------------------------------ |
| NOV10c | ------------------------------------------------------------ |
| NOV10d | ------------------------------------------------------------ |
| NOV10e | IRVSRKGWHMLLNLCFHIAMTSAVFAGGITLTNYQMVCQAVGITLHYSSLSTLLWMGVKA |
| NOV10f | ------------------------------------------------------------ |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | VAAWWSQEGPGEAGGWTSEGCQLRSSQPNVSALHCQHLGNVAVLMELSAFPREVGGAGAG |
| NOV10b | ------------------------------------------------------------ |
| NOV10c | ------------------------------------------------------------ |
| NOV10d | ------------------------------------------------------------ |
| NOV10e | RVLHKELTWHAPPPQEGDPALPTPSPMLRYILSIPALQLGRDSNAGVGNLPRFYLIAGGI |
| NOV10f | ------------------------------------------------------------ |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | LHPVVYPCTALLLLCLFATIITYILNHSSIRVSRKGWHMLLNLCFHIAMTSAVFAGGITL |
| NOV10b | ------------------------------------------------------------ |
| NOV10c | ------------------------------------------------------------ |
| NOV10d | ------------------------------------------------------------ |
| NOV10e | PLIICGITAAVNIHNYRDHSPYCWLVWRPSLGAFYIPVALILLITWIYFLCAGLRLRGPL |
| NOV10f | ------------------------------------------------------------ |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | TNYQMVCQAVGITLHYSSLSTLLWMGVKARVLHKELTWRAPPPQEGDPALPTPSPMLRYI |
| NOV10b | ------------------------------------------------------------ |
| NOV10c | ------------------------------------------------------------ |
| NOV10d | ------------------------------------------------------------ |
| NOV10e | AQNPKAGNSRASLEAGEELRGSTRLRGSGPLLSDSGSLLATGSARVGTPGPPEDGDSLYS |
| NOV10f | ------------------------------------------------------------ |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | LSIPALQLGRDSNAGVGNLPRFYLIAGGIPLIICGITAAVNIHNYRDHSPYCWLVWRPSL |
| NOV10b | ------------------------------------------------------------ |
| NOV10c | ------------------------------------------------------------ |
| NOV10d | ------------------------------------------------------------ |
| NOV10e | PGVQLGALVTTHFLYLAMWACGALAVSQRWLPRVVCSCLYGVAASALGLFVFTHHCARRR |
| NOV10f | ------------------------------------------------------------ |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | GAFYIPVALILLITWIYFLCAGLRLRGPLAQNPKAGNSRASLEAGEELRGSTRLRGSGPL |
| NOV10b | ------------------------------------------------------------ |
| NOV10c | ------------------------------------------------------------ |
| NOV10d | ------------------------------------------------------------ |
| NQV10e | DVEASWRACCPPASPAAPHAPPRAIPAAAEDGSPVFGEGPPSLKSSPSGSSGHPLALGPC |
| NOV10f | ------------------------------------------------------------ |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | LSDSGSLLATGSARVGTPGPPEDGDSLYSPGVQLGALVTTHFLYLAMWACGALAVSQRWL |
| NOV10b | ------------------------------------------------------------ |
| NOV10c | ------------------------------------------------------------ |
| NOV10d | ------------------------------------------------------------ |
| NOV10e | KLTNLQLAQSQVCEAGAAAGGEGEPEPAGTRGNLAHRHPNNVHHGRPAHKSRAKGHRAGE |
| NOV10f | ------------------------------------------------------------ |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | PRVVCSCLYGVAASALGLFVFTHHCARRRDVRASWRACCPPASPAAPHAPPRALPAAAED |
| NOV10b | ------------------------------------------------------------ |
| NOV10c | ------------------------------------------------------------ |
| NOV10d | ------------------------------------------------------------ |
| NOV10e | ACGKNRLKALRGGAAGALELLSSESGSLHNSPTDSYLGSSRNSPGAGLQLEGEPMLTPSE |
| NOV10f | ------------------------------------------------------------ |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | GSPVFGEGPPSLKSSPSGSSGHPLALGPCKLTNLQLAQSQVCEAGAAAGGEGEPEPAGTR |
| NOV10b | ------------------------------------------------------------ |
| NOV10c | ------------------------------------------------------------ |
| NOV10d | ------------------------------------------------------------ |
| NOV10e | GSDTSAAPLSEAGRAGQRRSASRDSLKGGGALEKESHRRSYPLNAASLNGAPKGGKYDDV |
| NOV10f | ------------------------------------------------------------ |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | GNLAHRHPNNVHHGRRAHKSRAKGHRAGEACGKNRLKALRGGAAGALELLSSESGSLHNS |
| NOV10b | ------------------------------------------------------------ |
| NOV10c | ------------------------------------------------------------ |
| NOV10d | ------------------------------------------------------------ |
| NOV10e | TLMGAEVASGGCMKTGLWKSETTV------------------------------------ |
| NOV10f | ------------------------------------------------------------ |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | PTDSYLGSSRNSPGAGLQLEGEPMLTPSEGSDTSAAPLSEAGRAGQRRSASRDSLKGGGA |
| NOV10b | ------------------------------------------------------------ |
| NOV10c | ------------------------------------------------------------ |
| NOV10d | ------------------------------------------------------------ |
| NOV10e | ------------------------------------------------------------ |
| NOV10f | ------------------------------------------------------------ |
| NOV10g | ------------------------------------------------------------ |
| NOV10h | ------------------------------------------------------------ |
| NOV10i | ------------------------------------------------------------ |
| NOV10j | ------------------------------------------------------------ |
| NOV10k | ------------------------------------------------------------ |
| NOV10a | LEKESHRRSYPLNAASLNGAPKGGKYDDVTLMGAEVASGGCMKTGLWKSETTV |
| NOV10b | ----------------------------------------------------- |
| NOV10c | ----------------------------------------------------- |
| NOV10d | ----------------------------------------------------- |
| NOV10e | ----------------------------------------------------- |
| NOV10f | ----------------------------------------------------- |
| NOV10g | ----------------------------------------------------- |
| NOV10h | ----------------------------------------------------- |
| NOV10i | ----------------------------------------------------- |
| NOV10j | ----------------------------------------------------- |
| NOV10k | ----------------------------------------------------- |
| NOV10a | (SEQ ID NO: 258) |
| NOV10b | (SEQ ID NO: 260) |
| NOV10c | (SEQ ID NO: 262) |
| NOV10d | (SEQ ID NO: 264) |
| NOV10e | (SEQ ID NO: 266) |
| NOV10f | (SEQ ID NO: 268) |
| NOV10g | (SEQ ID NO: 270) |
| NOV10h | (SEQ ID NO: 272) |
| NOV10i | (SEQ ID NO: 274) |
| NOV10j | (SEQ ID NO: 276) |
| NOV10k | (SEQ ID NO: 278) |

Further analysis of the NOV10a protein yielded the following properties shown in Table 10C.

TABLE 10C
Protein Sequence Properties NOV10a

| | |
|---|---|
| SignalP analysis: | Cleavage site between residues 27 and 28 |
| PSORT II analysis: | |

| PSG: | a new signal peptide prediction method | |
|---|---|---|
| | N-region: | length 7; pos. chg 2; neg. chg 0 |
| | H-region: | length 15; peak value 9.66 |
| | PSG score: | 5.26 |

| | |
|---|---|
| GvH: | von Heijne's method for signal seq. recognition |
| | GvH score (threshold: −2.1): 5.33 |
| | possible cleavage site: between 26 and 27 |

>>> Seems to have a cleavable signal peptide (1 to 26)

| ALOM: | Klein et al's method for TM region allocation | | |
|---|---|---|---|
| | Init position for calculation: 27 | | |
| | Tentative number of TMS(s) for the threshold 0.5: 5 | | |
| | INTEGRAL | Likelihood = | Transmembrane 958-974 |
| | | −7.91 | |
| | INTEGRAL | Likelihood = | Transmembrane 988-1004 |
| | | −1.22 | |
| | INTEGRAL | Likelihood = | Transmembrane 1093-1109 |
| | | −6.26 | |
| | INTEGRAL | Likelihood = | Transmembrane 1136-1152 |
| | | −8.17 | |
| | INTEGRAL | Likelihood = | Transmembrane 1252-1268 |
| | | −3.50 | |

| | |
|---|---|
| PERIPHERAL | Likelihood = 1.11 (at 1223) |

| | |
|---|---|
| | ALOM score: −8.17 (number of TMSs: 5) |
| MTOP: | Prediction of membrane topology (Hartmann et al.) |
| | Center position for calculation: 13 |
| | Charge difference: −2.0 C(1.0)-N(3.0) |
| | N >= C: N-terminal side will be inside |

>>> membrane topology: type 3a

| MITDISC: | discrimination of mitochondrial targeting seq | | | |
|---|---|---|---|---|
| | R content: | 2 | Hyd Moment(75): | 12.41 |
| | Hyd Moment(95): | 9.21 | G content: | 1 |
| | D/E content: | 1 | S/T content: | 0 |

| | |
|---|---|
| | Score: −2.41 |
| Gavel: | prediction of cleavage sites for mitochondrial preseq |
| | R-2 motif at 68 RRV\|VC |

NUCDISC: discrimination of nuclear localization signals

| | |
|---|---|
| | pat4: none |
| | pat7: PGPARRR (3) at 52 |
| | pat7: PARRRVV (5) at 54 |
| | pat7: PGKRRGV (5) at 849 |
| | bipartite: none |
| | content of basic residues: 9.1% |
| | NLS Score: 0.65 |
| KDEL: | ER retention motif in the C-terminus: none |

ER Membrane Retention Signals:

XXRR-like motif in the N-terminus: RGAP

none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 55.5 |
| COIL: | Lupas's algorithm to detect coiled-coil regions |
| | total: 0 residues |

Final Results (k = 9/23):

```
43.5%: endoplasmic reticulum
34.8%: nuclear
13.0%: mitochondrial
  4.3%: cytoplasmic
  4.3%: peroxisomal
```

>> prediction for CG50925-01 is end (k = 23)

A search of the NOV10a protein against the Geneseq database, a proprietary database that contains sequences published in patents and patent publication, yielded several homologous proteins shown in Table 10D.

TABLE 10D
Geneseq Results for NOV10a

| | | NOV10a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| ABU54447 | Human tumour endothelial marker | 263 . . . 1542 | 1231/1292 (95%) | 0.0 |
| | TEM 9 - Homo sapiens, 1331 aa. | 63 . . . 1331 | 1236/1292 (95%) | |
| | [WO200283874-A2, 24-OCT-2002] | | | |
| ABU54432 | Human tumour endothelial marker | 263 . . . 1542 | 1231/1292 (95%) | 0.0 |
| | TEM 5 - Homo sapiens, 1331 aa. | 63 . . . 1331 | 1236/1292 (95%) | |
| | [WO200283874-A2, 24-OCT-2002] | | | |
| AAU74824 | Human REPTR 7 protein - Homo | 263 . . . 1542 | 1231/1292 (95%) | 0.0 |
| | sapiens, 1331 aa. [WO200198354-A2, | 63 . . . 1331 | 1236/1292 (95%) | |
| | 27-DEC-2001] | | | |
| ABB90740 | Human Tumour Endothelial Marker | 263 . . . 1542 | 1231/1292 (95%) | 0.0 |
| | polypeptide SEQ ID NO 212 - Homo | 63 . . . 1331 | 1236/1292 (95%) | |
| | sapiens, 1331 aa. [WO200210217-A2, | | | |
| | 07-FEB-2002] | | | |
| ABB90725 | Human Tumour Endothelial Marker | 263 . . . 1542 | 1231/1292 (95%) | 0.0 |
| | polypeptide SEQ ID NO 188 - Homo | 63 . . . 1331 | 1236/1292 (95%) | |
| | sapiens, 1331 aa. [WO200210217-A2, | | | |
| | 07-FEB-2002] | | | |

In a BLAST search of public sequence databases, the NOV10a protein was found to have homology to the proteins shown in the BLASTP data in Table 10E.

TABLE 10E
Public BLASTP Results for NOV10a

| | | NOV10a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96PE1 | Tumor endothelial marker 5 | 263 . . . 1542 | 1231/1292 (95%) | 0.0 |
| | precursor - Homo sapiens | 63 . . . 1331 | 1236/1292 (95%) | |
| | (Human), 1331 aa. | | | |
| Q8TEM3 | FLJ00170 protein - Homo sapiens | 263 . . . 1542 | 1230/1292 (95%) | 0.0 |
| | (Human), 1310 aa (fragment). | 42 . . . 1310 | 1235/1292 (95%) | |
| Q91ZV8 | Tumor endothelial marker 5 | 280 . . . 1542 | 1083/1266 (85%) | 0.0 |
| | precursor - Mus musculus | 92 . . . 1329 | 1140/1266 (89%) | |
| | (Mouse), 1329 aa. | | | |
| Q9P1Z7 | KIAA1531 protein - Homo sapiens | 923 . . . 1542 | 587/620 (94%) | 0.0 |
| | (Human), 1206 aa (fragment). | 615 . . . 1206 | 589/620 (94%) | |
| Q8N3R1 | Hypothetical protein - Homo | 1031 . . . 1542 | 489/512 (95%) | 0.0 |
| | sapiens (Human), 489 aa | 1 . . . 489 | 489/512 (95%) | |
| | (fragment). | | | |

PFam analysis predicts that the NOV10a protein contains the domains shown in the Table 10F.

TABLE 10F
Domain Analysis of NOV10a

| | | Identities/ | |
|---|---|---|---|
| | | Similarities | |
| Pfam | NOV10a | for the | Expect |
| Domain | Match Region | Matched Region | Value |
| LRR | 314 . . . 337 | 10/25 (40%) | 0.0086 |
| | | 20/25 (80%) | |
| LRR | 338 . . . 361 | 10/25 (40%) | 0.15 |
| | | 20/25 (80%) | |
| LRRCT | 371 . . . 421 | 19/54 (35%) | 1.3e-08 |
| | | 40/54 (74%) | |
| HRM | 531 . . . 600 | 24/92 (26%) | 0.0088 |
| | | 48/92 (52%) | |
| GPS | 887 . . . 939 | 15/55 (27%) | 0.0041 |
| | | 34/55 (62%) | |
| 7tm_2 | 950 . . . 1280 | 65/359 (18%) | 0.0032 |
| | | 207/359 (58%) | |

### Example 11

The NOV11 clone was analyzed, and the nucleotide and encoded polypeptide sequences are shown in Table 11A.

TABLE 11A
NOV11 Sequence Analysis

| | | |
|---|---|---|
| NOV11a, CG51027-06 | SEQ ID NO: 281 | 1271 bp |
| DNA Sequence | ORF Start: ATG at 48 | ORF Stop: TAG at 1056 |

GCGCAGTGTGCTGGAATTCGCCCTTCCTCCGTGCCGCCTCTGCCGCGATGCCCCCCCCTGCGCCCGGC
GCCCGGCTCCGGCTTCTCGCCGCCGCCGCCCTGGCCGGCTTGGCCGTCATCAGCCGAGGGCTGCTCTC
CCAGAGCCTGGAGTTCAACTCTCCTGCCGACAACTACACAGTGTGTGAAGGTGACAACGCCACCCTCA
GCTGCTTCATCGACGAGCACGTGACCCGCGTGGCCTGGCTGAACCGCTCCAACATCCTGTATGCCGGC
AATGACCGCTGGACCAGCGACCCGCGGGTGCGGCTGCTCATCAACACCCCCGAGGAGTTCTCCATCCT
CATCACCGAGGTGGGGCTCGGCGACGAGGGCCTCTACACCTGCTCCTTCCAGACCCGCCACCAGCCGT
ACACCACTCAGGTCTACCTCATTGTCCACGTCCCTGCCCGCATTGTGAACATCTCGTCGCCTGTGATG
GTGAATGAGGGGGGCAATGTGAACCTGCTTTGCCTGGCCGTGGGGCGGCCAGAGCCCACGGTCACCTG
GAGACAGCTCCGAGACGGCTTCACCTCGGAGGGAGAGATCCTGGAGATCTCTGACATCCAGCGGGGCC
AGGCCGGGGAGTATGAGTGCGTGACTCACAACGGGGTTAACTCGGCGCCCGACAGCCGCCGCGTGCTG
GTCACAGTCAACTATCCTCCGACCATCACGGACGTGACCAGCGCCCGCACCGCGCTGGGCCGGGCCGC
CCTCCTGCGCTGCGAAGCCATGGCGGTTCCCCCCGCGGATTTCCAGTGGTATAAGGATGACAGACTGC
TGAGCAGCGGCACGGCCGAAGGCCTGAAGGTGCAGATGGAGCGCACCCGCTCGATGCTTCTCTTTGCC
AACGTGAGCGCCCGGCATTACGGCAACTATACGTGTTGCGCCGCCAACCGACTGGGAGCGTCCAGCGC
CTCCATGCGGCTCCTGTGCCCAGGATCCCTGGAGAACTCAGCCCCGAGGCCCCCAGGGCTCCTGGCCC
TCCTCTCCGCCCTGGGCTGGCTGTGGTGGAGAATGTAGGCGCAACCCAGTGGAGCTCGCCTCCCCCTG
CAGGGGGCCTCAGGCCAAGAGTGAGAGAAAAGGGGGAGCAAGAGCCCTGGGTCTCGTGGGGGCAGAAC
GGCTCTCGGCCACCAAGGAAGAAGAGAAGAGGAGAAAGAGGAGGAGGCAGAGGAAGAAAGATCTTTAG
AGAACCCATCACTGTGAGGGATAACGCAAAATTATGCATCTTTCTAC

| | | | |
|---|---|---|---|
| NOV11a, CG51027-06 | SEQ ID NO: 282 | 336 aa | MW at 36748.4kD |
| Protein Sequence | | | |

MPPPAPGARLRLLAAAALAGLAVISRGLLSQSLEFNSPADNYTVCEGDNATLSCFIDEHVTRVAWLNR
SNILYAGNDRWTSDPRVRLLINTPEEFSILITEVGLGDEGLYTCSFQTRHQPYTTQVYLIVHVPARIV
NISSPVMVNEGGNVNLLCLAVGRPEPTVTWRQLRDGFTSEGEILEISDIQRGQAGEYECVTHNGVNSA
PDSRRVLVTVNYPPTITDVTSARTALGRAALLRCEAMAVPPADFQWYKDDRLLSSGTAEGLKVQMERT
RSMLLFANVSARHYGNYTCCAANRLGASSASMRLLCPGSLENSAPRPPGLIJALLSALGWLWWRM

| | | |
|---|---|---|
| NOV11b, 278391205 | SEQ ID NO: 283 | 1033 bp |
| DNA Sequence | ORF Start: at 2 | ORF Stop: end of sequence |

CACCAAGCTTCCCACCATGCCCCCCCCTGCGCCCGGGGCCCGGCTCCGGCTTCTCGCCGCCGCCGCCC
TGGCCGGCTTGGCCGTCATCAGCCGAGGGCTGCTCTCCCAGAGCCTGGAGTTCAACTCTCCTGCCGAC
AACTACACAGTGTGTGAAGGTGACAACGCCACCCTCAGCTGCTTCATCGACGAGCACGTGACCCGCGT
GGCCTGGCTGAACCGCTCCAACATCCTGTATGCCGGCAATGACCGCTGGACCAGCGACCCGCGGGTGC
GGCTGCTCATCAACACCCCCGAGGAGTTCTCCATCCTCATCACCGAGGTGGGGCTCGGCGACGAGGGC
CTCTACACCTGCTCCTTCCAGACCCGCCACCAGCCGTACACCACTCAGGTCTACCTCATTGTCCACGT
CCCTGCCCGCATTGTGAACATCTCGTCGCCTGTGACGGTGAATGAGGGGGGCAATGTGAACCTGCTTT
GCCTGGCCGTGGGGCGGCCAGAGCCCACGGTCACCTGGAGACAGCTCCGAGACGGCTTCACCTCGGAG
GGAGAGATCCTGGAGATCTCTGACATCCAGCGGGGCCAGGCCGGGGAGTATGAGTGCGTGACTCACAA
CGGGGTTAACTCGGCGCCCGACAGCCGCCGCGTGCTGGTCACAGTCAACTATCCTCCGACCATCACGG
ACGTGACCAGCGCCCGCACCGCGCTGGGCCGGGCCGCCCTCCTGCGCTGCGAAGCCATGGCGGTTCCC
CCCGCGGATTTCCAGTGGTACAAGGATGACAGACTGCTGAGCAGCGGCACGGCCGAAGGCCTGAAGGT
GCAGACGGAGCGCACCCGCTCGATGCTTCTCTTTGCCAACGTGAGCGCCCGGCATTACGGCAACTATA
CGTGTCGCGCCGCCAACCGACTGGGAGCGTCCAGCGCCTCCATGCGGCTCCTGCGCCCAGGATCCCTG
GAGAACTCAGCCCCGAGGCCCCCAGGGCTCCTGGCCCTCCTCTCCGCCCTGGGCTGGCTGTGGTGGAG
AATGCTCGAGGGC

| | | | |
|---|---|---|---|
| NOV11b, 278391205 | SEQ ID NO: 284 | 344 aa | MW at 37634.3kD |
| Protein Sequence | | | |

TKLPTMPPPAPGARLRLLAAAALAGLAVISRGLLSQSLEFNSPADNYTVCEGDNATLSCFIDEHVTRV
AWLNRSNILYAGNDRWTSDPRVRLLINTPEEFSILITEVGLGDEGLYTCSFQTRHQPYTTQVYLIVHV
PARIVNISSPVTVNEGGNVNLLCLAVGRPEPTVTWRQLRDGFTSEGEILEISDIQRGQAGEYECVTHN
GVNSAPDSPRVLVTVNYPPTITDVTSARTALGRAALLRCEAMAVPPADFQWYKDDRLLSSGTAEGLKV
QTERTRSMLLFANVSARHYGNYTCRAANRLGASSASMRLLRPGSLENSAPRPPGLLALLSALGWLWWR
MLEG

| | | |
|---|---|---|
| NOV11c, 278391050 | SEQ ID NO: 285 | 976 bp |
| DNA Sequence | ORF Start: at 2 | ORF Stop: end of sequence |

CACCAAGCTTCTGGCCGGCTTGGCCGTCATCAGCCGAGGGCTGCTCTCCCAGAGCCTGGAGTTCAACT
CTCCTGCCGACAACTACACAGTGTGTGAAGGTGACAACGCCACCCTCAGCTGCTTCATCGACGAGCAC
GTGACCCGCGTGGCCTGGCTGAACCGCTCCAACATCCTGTATGCCGGCAATGACCGCTGGACCAGCGA
CCCGCGGGTGCGGCTGCTCATCAACACCCCCGAGGAGTTCTCCATCCTCATCACCGAGGTGGGGCTCG
GCGACGAGGGCCTCTACACCTGCTCCTTCCAGACCCGCCACCAGCCGTACACCACTCAGGTCTACCTC
ATTGTCCACGTCCCTGCCCGCATTGTGAACATCTCGTCGCCTGTGACGGTGAATGAGGGGGGCAATGT
GAACCTGCTTTGCCTGGCCGTGGGGCGGCCAGAGCCCACGGTCACCTGGAGACAGCTCCGAGACGGCT
TCACCTCGGAGGGAGAGATCCTGGAGATCTCTGACATCCAGCGGGGCCAGGCCGGGGAGTATGAGTGC
GTGACTCACAACGGGGTTAACTCGGCGCCCGACAGCCGCCGCGTGCTGGTCACAGTCAACTATCCTCC
GACCATCACGGACGTGACCAGCGCCCGCACCGCGCTGGGCCGGGCCGCCCTCCTGCGCTGCGAAGCCA
TGGCGGTTCCCCCCGCGGATTTCCAGTGGTACAAGGATGACAGACTGCTGAGCAGCGGCACGGCCGAA
GGCCTGAAGGTGCAGACGGAGCGCACCCGCTCGATGCTTCTCTTTGCCAACGTGAGCGCCCGGCATTA
CGGCAACTATACGTGTCGCGCCGCCAACCGACTGGGAGCGTCCAGCGCCTCCATGCGGCTCCTGCGCC
CAGGATCCCTGGAGAACTCAGCCCCGAGGCCCCCAGGGCTCCTGGCCCTCCTCTCCGCCCTGGGCTGG
CTGTGGTGGAGAATGCTCGAGGGC

| | | | |
|---|---|---|---|
| NOV11c, 278391050 | SEQ ID NO: 286 | 325 aa | MW at 35781.1kD |
| Protein Sequence | | | |

TKLLAGLAVISRGLLSQSLEFNSPADNYTVCEGDNATLSCFIDEHVTRVAWLNRSNILYAGNDRWTSD
PRVRLLINTPEEFSILITEVGLGDEGLYTCSFQTRHQPYTTQVYLIVHVPARIVNISSPVTVNEGGNV
NLLCLAVGRPEPTVTWRQLRDGFTSEGEILEISDIQRGQAGEYECVTHNGVNSAPDSRRVLVTVNYPP
TITDVTSARTALGRAALLRCEAMAVPPADFQWYKDDRLLSSGTAEGLKVQTERTRSMLLFANVSARHY
GNYTCRAANRLGASSASMRLLRPGSLENSAPRPPGLLALLSALGWLWWRMLEG

| | | |
|---|---|---|
| NOV11d, 277582210 | SEQ ID NO: 287 | 1760 bp |
| DNA Sequence | ORF Start: at 2 | ORF Stop: end of sequence |

CACCAAGCTTGGTGACAACGCCACCCTCAGCTGCTTCATCGACGAGCACGTGACCCGCGTGGCCTGGC
TGAACCGCTCCAACATCCTGTATGCCGGCAATGACCGCTGGACCAGCGACCCGCGGGTGCGGCTGCTC
ATCAACACCCCCGAGGAGTTCTCCATCCTCATCACCGAGGTGGGGCTCGGCGACGAGGGCCTCTACAC
CTGCTCCTTCCAGACCCGCCACCAGCCGTACACCACTCAGGTCTACCTCATTGTCCACGTCCCTGCCC
GCATTGTGAACATCTCGTCGCCTGTGACGGTGAATGAGGGGGGCAATGTGAACCTGCTTTGCCTGGCC
GTGGGGCGGCCAGAGCCCACGGTCACCTGGAGACAGCTCCGAGACGGCTTCACCTCGGAGGGAGAGAT
CCTGGAGATCTCTGACATCCAGCGGGGCCAGGCCGGGGAGTATGAGTGCGTGACTCACAACGGGGTTA
ACTCGGCGCCCGACAGCCGCCGCGTGCTGGTCACAGTCAACTATCCTCCGACCATCACGGACGTGACC
AGCGCCCGCACCGCGCTGGGCCGGGCCGCCCTCCTGCGCTGCGAAGCCATGGCGGTTCCCCCCGCGGA
TTTCCAGTGGTACAAGGATGACAGACTGCTGAGCAGCGGCACGGCCGAAGGCCTGAAGGTGCAGACGG
AGCGCACCCGCTCGATGCTTCTCTTTGCCAACGTGAGCGCCCGGCATTACGGCAACTATACGTGTCGC
GCCCTCGAGGGC

| | | | |
|---|---|---|---|
| NOV11d, 277582210 | SEQ ID NO: 288 | 253 aa | MW at 28074.3kD |
| Protein Sequence | | | |

TKLGDNATLSCFIDEHVTRVAWLNRSNILYAGNDRWTSDPRVRLLINTPEEFSILITEVGLGDEGLYT
CSFQTRHQPYTTQVYLIVHVPARIVNISSPVTVNEGGNVNLLCLAVGRPEPTVTWRQLRDGFTSEGEI
LEISDIQRGQAGEYECVTHNGVNSAPDSRRVLVTVNYPPTITDVTSARTALGRAALLRCEAMAVPPAD
FQWYKDDRLLSSGTAEGLKVQTERTRSMLLFANVSARHYGNYTCRALEG

| | | |
|---|---|---|
| NOV11e, 277582173 | SEQ ID NO: 289 | 946 bp |
| DNA Sequence | ORF Start: at 2 | ORF Stop: end of sequence |

CACCAAGCTTCTGCTCTCCCAGAGCCTGGAGTTCAACTCTCCTGCCGACAACTACACAGTGTGTGAAG
GTGACAACGCCACCCTCAGCTGCTTCATCGACGAGCACGTGACCCGCGTGGCCTGGCTGAACCGCTCC
AACATCCTGTATGCCGGCAATGACCGCTGGACCAGCGACCCGCGGGTGCGGCTGCTCATCAACACCCC
CGAGGAGTTCTCCATCCTCATCACCGAGGTGGGGCTCGGCGACGAGGGCCTCTACACCTGCTCCTTCC
AGACCCGCCACCAGCCGTACACCACTCAGGTCTACCTCATTGTCCACGTCCCTGCCCGCATTGTGAAC
ATCTCGTCGCCTGTGACGGTGAATGAGGGGGGCAATGTGAACCTGCTTTGCCTGGCCGTGGGGCGGCC
AGAGCCCACGGTCACCTGGAGACAGCTCCGAGACGGCTTCACCTCGGAGGGAGAGATCCTGGAGATCT
CTGACATCCAGCGGGGCCAGGCCGGGGAGTATGAGTGCGTGACTCACAACGGGGTTAACTCGGCGCCC
GACAGCCGCCGCGTGCTGGTCACAGTCAACTATCCTCCGACCATCACGGACGTGACCAGCGCCCGCAC
CGCGCTGGGCCGGGCCGCCCTCCTGCGCTGCGAAGCCATGGCGGTTCCCCCCGCGGATTTCCAGTGGT
ACAAGGATGACAGACTGCTGAGCAGCGGCACGGCCGAAGGCCTGAAGGTGCAGACGGAGCGCACCCGC
TCGATGCTTCTCTTTGCCAACGTGAGCGCCCGGCATTACGGCAACTATACGTGTCGCGCCGCCAACCG
ACTGGGAGCGTCCAGCGCCTCCATGCGGCTCCTGCGCCCAGGATCCCTGGAGAACTCAGCCCCGAGGC
CCCCAGGGCTCCTGGCCCTCCTCTCCGCCCTGGGCTGGCTGTGGTGGAGAATGCTCGAGGGC

| | | | |
|---|---|---|---|
| NOV11e, 277582173 | SEQ ID NO: 290 | 315 aa | MW at 34843.0kD |
| Protein Sequence | | | |

TKLLLSQSLEFNSPADNYTVCEGDNATLSCFIDEHVTRVAWLNRSNILYAGNDRWTSDPRVRLLINTP
EEFSILITEVGLGDEGLYTCSFQTRHQPYTTQVYLIVHVPARIVNISSPVTVNEGGNVNLLCLAVGRP
EPTVTWRQLRDGFTSEGEILEISDIQRGQAGEYECVTHNGVNSAPDSRRVLVTVNYPPTITDVTSART
ALGRAALLRCEAMAVPPADFQWYKDDRLLSSGTAEGLKVQTERTRSMLLFANVSARHYGNYTCRAANR
LGASSASMELLRPGSLENSAPRPPGLLALLSALGWLWWRMLEG

| | | |
|---|---|---|
| Nov11f, CG51027-09 | SEQ ID NO: 291 | 976 bp |
| DNA Sequence | ORF Start: at 11 | ORF Stop: at 968 |

CACCAAGCTTCTGGCCGGCTTGGCCGTCATCAGCCGAGGGCTGCTCTCCCAGAGCCTGGAGTTCAACT
CTCCTGCCGACAACTACACAGTGTGTGAAGGTGACAACGCCACCCTCAGCTGCTTCATCGACGAGCAC
GTGACCCGCGTGGCCTGGCTGAACCGCTCCAACATCCTGTATGCCGGCAATGACCGCTGGACCAGCGA
CCCGCGGGTGCGGCTGCTCATCAACACCCCCGAGGAGTTCTCCATCCTCATCACCGAGGTGGGGCTCG
GCGACGAGGGCCTCTACACCTGCTCCTTCCAGACCCGCCACCAGCCGTACACCACTCAGGTCTACCTC
ATTGTCCACGTCCCTGCCCGCATTGTGAACATCTCGTCGCCTGTGACGGTGAATGAGGGGGGCAATGT
GAACCTGCTTTGCCTGGCCGTGGGGCGGCCAGAGCCCACGGTCACCTGGAGACAGCTCCGAGACGGCT
TCACCTCGGAGGGAGAGATCCTGGAGATCTCTGACATCCAGCGGGGCCAGGCCGGGGAGTATGAGTGC
GTGACTCACAACGGGGTTAACTCGGCGCCCGACAGCCGCCGCGTGCTGGTCACAGTCAACTATCCTCC
GACCATCACGGACGTGACCAGCGCCCGCACCGCGCTGGGCCGGGCCGCCCTCCTGCGCTGCGAAGCCA
TGGCGGTTCCCCCCGCGGATTTCCAGTGGTACAAGGATGACAGACTGCTGAGCAGCGGCACGGCCGAA
GGCCTGAAGGTGCAGACGGAGCGCACCCGCTCGATGCTTCTCTTTGCCAACGTGAGCGCCCGGCATTA
CGGCAACTATACGTGTCGCGCCGCCAACCGACTGGGAGCGTCCAGCGCCTCCATGCGGCTCCTGCGCC
CAGGATCCCTGGAGAACTCAGCCCCGAGGCCCCCAGGGCTCCTGGCCCTCCTCTCCGCCCTGGGCTGG
CTGTGGTGGAGAATGCTCGAGGGC

| | | | |
|---|---|---|---|
| NOV11f, CG51027-09 | SEQ ID NO: 292 | 319 aa | MW at 35139.3kD |
| Protein Sequence | | | |

LAGLAVISRGLLSQSLEFNSPADNYTVCEGDNATLSCFIDENVTRVAWLNRSNILYAGNDRWTSDPRV
RLLINTPEEFSILITEVGLGDEGLYTCSFQTRHQPYTTQVYLIVHVPARIVNISSPVTVNEGGNVNLL
CLAVGRPEPTVTWRQLRDGFTSEGEILEISDIQRGQAGEYECVTHNGVNSAPDSRRVLVTVNYPPTIT
DVTSARTALGRAALLRCEAMAVPPADFQWYKDDRLLSSGTAEGLKVQTERTRSMLLFANVSARHYGNY
TCRAANRLGASSASMRLLRPGSLENSAPRPPGLLALLSALGWLWWRM

| | | |
|---|---|---|
| NOV11g, 316763704 | SEQ ID NO: 293 | 976 bp |
| DNA Sequence | ORF Start: at 2 | ORF Stop: end of sequence |

CACCTCGCGACTGGCCGGCTTGGCCGTCATCAGCCGAGGGCTGCTCTCCCAGAGCCTGGAGTTCAACT
CTCCTGCCGACAACTACACAGTGTGTGAAGGTGACAACGCCACCCTCAGCTGCTTCATCGACGAGCAC
GTGACCCGCGTGGCCTGGCTGAACCGCTCCAACATCCTGTATGCCGGCAATGACCGCTGGACCAGCGA
CCCGCGGGTGCGGCTGCTCATCAACACCCCCGAGGAGTTCTCCATCCTCATCACCGAGGTGGGGCTCG
GCGACGAGGGCCTCTACACCTGCTCCTTCCAGACCCGCCACCAGCCGTACACCACTCAGGTCTACCTC
ATTGTCCACGTCCCTGCCCGCATTGTGAACATCTCGTCGCCTGTGACGGTGAATGAGGGGGGCAATGT
GAACCTGCTTTGCCTGGCCGTGGGGCGGCCAGAGCCCACGGTCACCTGGAGACAGCTCCGAGACGGCT
TCACCTCGGAGGGAGAGATCCTGGAGATCTCTGACATCCAGCGGGGCCAGGCCGGGGAGTATGAGTGC
GTGACTCACAACGGGGTTAACTCGGCGCCCGACAGCCGCCGCGTGCTGGTCACAGTCAACTATCCTCC
GACCATCACGGACGTGACCAGCGCCCGCACCGCGCTGGGCCGGGCCGCCCTCCTGCGCTGCGAAGCCA
TGGCGGTTCCCCCCGCGGATTTCCAGTGGTACAAGGATGACAGACTGCTGAGCAGCGGCACGGCCGAA
GGCCTGAAGGTGCAGACGGAGCGCACCCGCTCGATGCTTCTCTTTGCCAACGTGAGCGCCCGGCATTA
CGGCAACTATACGTGTCGCGCCGCCAACCGACTGGGAGCGTCCAGCGCCTCCATGCGGCTCCTGCGCC
CAGGATCCCTGGAGAACTCAGCCCCGAGGCCCCCAGGGCTCCTGGCCCTCCTCTCCGCCCTGGGCTGG
CTGTGGTGGAGAATGGTCGACGGC

| | | | |
|---|---|---|---|
| NOV11g, 316763704 | SEQ ID NO: 294 | 325 aa | MW at 35755.0kD |
| Protein Sequence | | | |

TSRLAGLAVISRGLLSQSLEFNSPADNYTVCEGDNATLSCFIDEHVTRVAWLNRSNILYAGNDRWTSD
PRVRLLINTPEEFSILITEVGLGDEGLYTCSFQTRHQPYTTQVYLIVHVPARIVNISSPVTVNEGGNV
NLLCLAVGRPEPTVTWRQLRDGFTSEGEILEISDIQRGQAGEYECVTHNGVNSAPDSRRVLVTVNYPP
TITDVTSARTALGRAALLRCEAMAVPPADFQWYKDDRLLSSGTAEGLKVQTERTRSMLLFAINSARHY
GNYTCRAANRLGASSASMRLLRPGSLENSAPRPPGLLALLSALGWLWWRMVDG

| | | |
|---|---|---|
| NOV11h, CG51027-01 | SEQ ID NO: 295 | 1018 bp |
| DNA Sequence | ORF Start: at 1 | ORF Stop: TAG at 958 |

CTGGCCGGCTTGGCCGTCATCAGCCGAGGGCTGCTCTCCCAGAGCCTGGAGTTCAACTCTCCTGCCGA
CAACTACACAGTGTGTGAAGGTGACAACGCCACCCTCAGCTGCTTCATCGACGAGCACGTGACCCGCG
TGGCTTGGCTGAACCGCTCCAACATCCTGTATGCCGGCAATGACCGCTGGACCAGCGACCCGCGGGTG
CGGCTGCTCATCAACACCCCCGAGGAGTTCTCCATCCTCATCACCGAGGTGGGGCTCGGCGACGAGGG
CCTCTACACCTGCTCCTTCCAGACCCGCCACCAGCCGTACACCACTCAGGTCTACCTCATTGTCCACG
TCCCTGCCCGCATTGTGAACATCTCGTCGCCTGTGACGGTGAATGAGGGGGGCAATGTGAACCTGCTT
TGCCTGGCCGTGGGGCGGCCAGAGCCCACGGTCACCTGGAGACAGCTCCGAGACGGCTTCACCTCGGA
GGGAGAGATCCTGGAGATCTCTGACATCCAGCGGGGCCAGGCCGGGGAGTATGAGTGCGTGACTCACA
ACGGGGTTAACTCGGCGCCCGACAGCCGCCGCGTGCTGGTCACAGTCAACTATCCTCCGACCATCACG
GACGTGACCAGCGCCCGCACCGCGCTGGGCCGGACCGCCCTCCTGCGCTGCGAAGCCATGGCGGTTCC
CCCCGCGGATTTCCAGTGGTACAAGGATGACAGACTGCTGAGCAGCGGCACGGCCGAAGGCCTGAAGG
TGCAGACGGAGCGCACCCGCTCGATGCTTCTCTTTGCCAACGTGAGCGCCCGGCATTACGGCAACTAT
ACGTGTCGCGCCGCCAATCGACTGGGAGCGTCCAGCGCCTCCATGCGGCTCCTGCGCCCAGGATCCCT
GGAGAACTCAGCCCCGAGGCCCCCAGGGCTCCTGGCCCTCCTCTCCGCCCTGGGCTGGCTGTGGTGGA
GAATGTAGGCGCAACCCAGTCCAGCTCACCTCCCCCTGCAGGGGGCCTCAAACCAAGAGTGAGAGA

| | | | |
|---|---|---|---|
| NOV11h, CG51027-01 | SEQ ID NO: 296 | 319 aa | MW at 35169.4kD |
| Protein Sequence | | | |

LAGLAVISRGLLSQSLEFNSPADNYTVCEGDNATLSCFIDEHVTRVAWLNRSNILYAGNDRWTSDPRV
RLLINTPEEFSILITEVGLGDEGLYTCSFQTRHQPYTTQVYLIVHVPARIVNISSPVTVNEGGNVNLL
CLAVGRPEPTVTWRQLRDGFTSEGEILEISDIQRGQAGEYECVTHNGVNSAPDSRRVLVTVNYPPTIT
DVTSARTALGRTALLRCEAMAVPPADFQWYKDDRLLSSGTAEGLKVQTERTRSMLLFANVSARHYGNY
TCRAANRLGASSASMRLLRPGSLENSAPRPPGLLALLSALGWLWWRM

| | | |
|---|---|---|
| NOV11i, CG51027-02 | SEQ ID NO: 297 | 926 bp |
| DNA Sequence | ORF Start: at 1 | ORF Stop: TAG at 922 |

TCCCAGAGCCTGGAGTTCAACTCTCCTGCCGACAACTACACAGTGTGTGAAGGTGACAACGCCACCCT
CAGCTGCTTCATCGACGAGCACGTGACCCGCGTGGCCTGGCTGAACCGCTCCAACATCCTGTATGCCG
GCAATGACCGCTGGACCAGCGACCCGCGGGTGCGGCTGCTCATCAACACCCCCGAGGAGTTCTCCATC
CTCATCACCGAGGTGGGGCTCGGCGACGAGGGCCTCTACACCTGCTCCTTCCAGACCCGCCACCAGCC
GTACACCACTCAGGTCTACCTCATTGTCCACGTCCCTGCCCGCATTGTGAACATCTCGTCGCCTGTGA
CGGTGAATGAGGGGGGCAATGTGAACCTGCTTTGCCTGGCCGTGGGGCGGCCAGAGCCCACGGTCACC
TGGAGACAGCTCCGAGACGGCTTCACCTCGGAGGGAGAGATCCTGGAGATCTCTGACATCCAGCGGGG
CCAGGCCGGGGAGTATGAGTGCGTGACTCACAACGGGGTTAACTCGGCGCCCGACAGCCGCCGCGTGC
TGGTCACAGTCAACTATCCTCCGACCATCACGGACGTGACCAGCGCCCGCACCGCGCTGGGCCGGGCC
GCCCTCCTGCGCTGCGAAGCCATGGCGGTTCCCCCCGCGGATTTCCAGTGGTACAAGGATGACAGACT
GCTGAGCAGCGGCACGGCCGAAGGCCTGAAGGTGCAGACGGAGCGCACCCGCTCGATGCTTCTCTTTG
CCAACGTGAGCGCCCGGCATTACGGCAACTATACGTGTCGCGCCGCCAACCGACTGGGAGCGTCCAGC
GCCTCCATGCGGCTCCTGCGCCCAGGATCCCTGGAGAACTCAGCCCCGAGGCCCCCAGGGCTCCTGGC
CCTCCTCTCCGCCCTGGGCTGGCTGTGGTGGAGAATGTAGGC

| | | | |
|---|---|---|---|
| NOV11i, CG51027-02 | SEQ ID NO: 298 | 307 aa | MW at 33974.9kD |
| Protein Sequence | | | |

SQSLEFNSPADNYTVCEGDNATLSCFIDEHVTRVAWLNRSNILYAGNDRWTSDPRVRLLINTPEEFSI
LITEVGLGDEGLYTCSFQTRHQPYTTQVYLIVHVPARIVNISSPVTVNEGGNVNLLCLAVGRPEPTVT
WRQLRDGFTSEGEILEISDIQRGQAGEYECVTHNGVNSAPDSRRVLVTVNYPPTITDVTSARTALGRA
ALLRCEAMAVPPADFQWYKDDRLLSSGTAEGLKVQTERTRSMLLFANVSARHYGNYTCRAANRLGASS
ASMRLLRPGSLENSAPRPPGLLALLSALGWLWWRM

| | | |
|---|---|---|
| NOV11j, CG51027-03 | SEQ ID NO: 299 | 1171 bp |
| DNA Sequence | ORF Start: at 1 | ORF Stop: TAG at 958 |

CTGGCCGGCTTGGCCGTCATCAGCCGAGGGCTGCTCTCCCAGAGCCTGGAGTTCAACTCTCCTGCCGA
CAACTACACAGTGTGTGAAGGTGACAACGCCACCCTCAGCTGCTTCATCGACGAGCACGTGACCCGCG
TGGCCTGGCTGAACCGCTCCAACATCCTGTATGCCGGCAATGACCGCTGGACCAGCGACCCGCGGGTG
TGGCTGCTCATCAACACCCCCGAGGAGTTCTCCATCCTCATCACCGAGGTGGGGCTCGGCGACGAGGG
CCTCTACACCTGCTCCTTCCAGACCCGCCACCAGCCGTACACCACTCAGGTCTACCTCATTGTCCACG
TCCCTGCCCGCATTGTGAACATCTCGTCGCCTGTGATGGTGAATGAGGGGGGCAATGTGAACCTGCTT
TGCCTGGCCGTGGGGCGGCCAGAGCCCACGGTCACCTGGAGACAGCTCCGAGACGGCTTCACCTCGGA
GGGAGAGATCCTGGAGATCTCTGACATCCAGCGGGGCCAGGCCGGGGAGTATGAGTGCGTGACTCACA
ACGGGGTTAACTCGGCGCCCGACAGCCGCCGCGTGCTGGTCACAGTCAACTATCCTCCGACCATCACG
GACGTGACCAGCGCCCGCACCGCGCTGGGCCGGGCCGCCCTCCTGCGCTGCGAAGCCATGGCGGTTCC
CCCCGCGGATTTCCAGTGGTATAAGGATGACAGACTGCTGAGCAGCGGCACGGCCGAAGGCCTGAAGG
TGCAGATGGAGCGCACCCGCTCGATGCTTCTCTTTGCCAACGTGAGCGCCCGGCATTACGGCAACTAT
ACGTGTCGCGCCGCCAACCGACTGGGAGCGTCCAGCGCCTCCATGCGGCTCCTGCGCCCAGGATCCCT
GGAGAACTCAGCCCCGAGGCCCCCAGGGCTCCTGGCCCTCCTCTCCGCCCTGGGCTGGCTGTGGTGGA
GAATGTAGGCGCAACCCAGTGGAGCTCACCTCCCCCTGCAGGGGGCCTCAGGCCAAGAGTGAGAGAAA
CGGGGGAGCAAGAGCCGTGGGTCTCGTGGGGGCAGAAGAGCTCTCGGCCACCAAGGAAGAAGAGAGAG
GAGAAGAGGAGGAGGCAGAGGAAGAAAGATCTTCAGAGAACCCATCACTGTGAGGGATAACGCAAAAT
TATGCATCTTTCTAC

| | | | |
|---|---|---|---|
| NOV11j, CG51027-03 | SEQ ID NO: 300 | 319 aa | MW at 35229.6kD |
| Protein Sequence | | | |

LAGLAVISRGLLSQSLEFNSPADNYTVCEGDNATLSCFIDEHVTRVAWLNRSNILYAGNDRWTSDPRV
WLLINTPEEFSILITEVGLGDEGLYTCSFQTRHQPYTTQVYLIVHVPARIVNISSPVMVNEGGNVNLL
CLAVGRPEPTVTWRQLRDGFTSEGEILEISDIQRGQAGEYECVTHNGVNSAPDSRRVLVTVNYPPTIT
DVTSARTALGRAALLRCEAMAVPPADFQWYKDDRLLSSGTAEGLKVQMERTRSMLLFANVSARHYGNY
TCRAACRLGASSASMRLLRPGSLENSAPRPPGLLALLSALSWLWWRM

| | | |
|---|---|---|
| NOV11k, CG51027-04 | SEQ ID NO: 301 | 927 bp |
| DNA Sequence | ORF Start: at 1 | ORF Stop: end of sequence |

TTGGCCGTCATCAGCCGAGGGCTGCTCTCCCAGAGCCTGGAGTTCAACTCTCCTGCCGACAACTACAC
AGTGTGTGAAGGTGACAACGCCACCCTCAGCTGCTTCATCGACGAGCACGTGACCCGCGTGGCCTGGC
TGAACCGCTCCAACATCCTGTATGCCGGCAATGACCGCTGGACCAGCGACCCGCGGGTGCGGCTGCTC
ATCAACACCCCCGAGGAGTTCTCCATCCTCATCACCGAGGTGGGGCTCGGCGACGAGGGCCTCTACAC
CTGCTCCTTCCAGACCCGCCACCAGCCGTACACCACTCAGGTCTACCTCATTGTCCACGTCCCTGCCC
GCATTGTGAACATCTCGTCGCCTGTGACGGTGAATGAGGGGGGCAATGTGAACCTGCTTTGCCTGGCC
GTGGGGCGGCCAGAGCCCACGGTCACCTGGAGACAGCTCCGAGACGGCTTCACCTCGGAGGGAGAGAT
CCTGGAGATCTCTGACATCCAGCGGGGCCAGGCCGGGGAGTATGAGTGCGTGACTCACAACGGGGTTA
ACTCGGCGCCCGACAGCCGCCGCGTGCTGGTCACAGTCAACTATCCTCCGACCATCACGGACGTGACC
AGCGCCCGCACCGCGCTGGGCCGGGCCGCCCTCCTGCGCTGCGAAGCCATGGCGGTTCCCCCCGCGGA
TTTCCAGTGGTACAAGGATGACAGACTGCTGAGCAGCGGCACGGCCGAAGGCCTGAAGGTGCAGACGG
AGCGCACCCGCTCGATGCTTCTCTTTGCCAACGTGAGCGCCCGGCATTACGGCAACTATACGTGTCGC
GCCGCCAACCGACTGGGAGCGTCCAGCGCCTCCATGCGGCTCCTGCGCCCAGGATCCCTGGAGAACTC
AGCCCCGAGGCCCCCAGGGCTCCTGGCCCTCCTCTCCGCCCTG

| | | | |
|---|---|---|---|
| NOV11k, CG51 027-04 | SEQ ID NO: 302 | 309 aa | MW at 33881.8kD |
| Protein Sequence | | | |

LAVISRGLLSQSLEFNSPADNYTVCEGDNATLSCFIDEHVTRVAWLNRSNILYAGNDRWTSDPRVRLL
INTPEEFSILITEVGLGDEGLYTCSFQTRHQPYTTQVYLIVHVPARIVNISSPVTVNEGGNVNLLCLA
VGRPEPTVTWRQLRDGFTSEGEILEISDIQRGQAGEYECVTHNGVNSAPDSRRVLVTVNYPPTITDVT
SARTALGRAALLRCEAMAVPPADFQWYKDDRLLSSGTAEGLKVQTERTRSMLLFANVSARHYGNYTCR
AANRLGASSASMRLLRPGSLENSAPRPPGLLALLSAL

| | | |
|---|---|---|
| NOV11l, CG51027-05 | SEQ ID NO: 303 | 1169 bp |
| DNA Sequence | ORF Start: ATG at 71 | ORF Stop: TAG at 1079 |

CTCCAACAAGCATAGCGCGCCCCGGACCGGCCCCCCCTTTCCCCTCCCCCTCCGTGCCGCCTCTGCCG
CGATGCCCCCCGCTGCGCCCGGGGCCCGGCTCCGGCTTCTCGCCGCCGCCGCCCTGGCCGGCTTGGCC
GTCATCAGCCGGGGGCTGCTCTCCCAGAGGCTGGAGTTCAACTCTCCTGCCGACAACTACACAGTGTG
TGAAGGTGACAACGCCACCCTCAGCTGCTTCATGGACGAGCATGTGACCCGCGTGGCCTGGCTGAACC
GCTCCAACATCCTGTACGCCGGCAACGACCGCAGGACCAGGGACCCGCGGGTGCGGCTGCTCATCAAC
ACCCCCGAGGAGTTCTCCATCCTCGTCACCGAGGTGGGGCTCGGCGACGAGGGCCTCTACACCTGCTC
CTTCCAGACCCGCCACCAGCCGTACACCACTCAGGTCTACCTCATTGTCCACGTCCCTGCCCGCGTTG
TGAACATCTCGTCGCCTGTGATGGTGAATGAGGGAGGTAATGTGAACCTGCTTTGCCTGGCCGTGGGG
CGGCCAGAGCCCACGGTCACCTGGAGACAGCTCCGAGACGGCTTCACCTCGGAGGGAGAGATCCTGGA
GATCTCTGACATCCTGCGGGGCCAGGCCGGGGAGTATGAGTGCGTGACTCACAACGGGGTTAACTCGG
CGCCCGACAGCCGCCGCGTGCTGGTCACAGTCAACTATCCTCCGACCATCACGGACGT9ACCAGCGCC
CGCACCGCGCTGGGGCCGGGCCGCCTACTGCGCTGCGAAGCCATGGCGGTTTCCCCCGCGGATTTCCA
GTGGTATAAGGATGACAGACTACTGAGCAGCGGCACGGCCGAGGGCCTGAAGGTGCAGATGGAGCGCA
CTCGCTCGATGCTTCTCTTTGCCAACATGAGCGCCCGGCATTACGGCAACTATACGTGTTGCGCCGCC
AACCGGCTGGGAGCGTCCAGCGCCTCCATGCGGCTCCTGTGCCCAGGATCCCTGGAGAACTCAGCCCC
GAGGCCCCCAGGGCCCCTGGCCCTCCTCTCCGCCCTGGGCTGGCTGTGGTGGAGAATGTAGGCGCAAC
CCAGTGGAGCTCGCCTCCCCCTGCAGGGGGCCTCAGGCCAAGAGTGAGAGAAAAGGGGGAGCAAGAGC
CCTGGGTCTCGTG

| | | | |
|---|---|---|---|
| NOV11l, CG51027-05 | SEQ ID NO: 304 | 336 aa | MW at 36823.6kD |
| Protein Sequence | | | |

MPPAAPGARLRLLAAAALAGLAVISRGLLSQRLEFNSPADNYTVCEGDNATLSCFMDEHVTRVAWLNR
SNILYAGNDRRTRDPRVRLLINTPEEFSILVTEVGLGDEGLYTCSFQTRHQPYTTQVYLIVHVPARVV
NISSPVMVNEGGNVNLLCLAVGRPEPTVTWRQLRDGFTSEGEILEISDILRGQAGEYECVTHNGVNSA
PDSRRVLVTVNYPPTITDVTSARTALGPGRLLRCEAMAVSPADFQWYKDDRLLSSGTAEGLKVQMERT
RSMLLFANMSARHYGNYTCCAANRLGAS SASMRLLCPGSLENSAPRPPGPLALLSALGWLWWRM

| | | |
|---|---|---|
| NOV11m, CG51027-07 | SEQ ID NO: 305 | 946 bp |
| DNA Sequence | ORF Start: at 11 | ORF Stop: at 938 |

CACCAAGCTTCTGCTCTCCCAGAGCCTGGAGTTCAACTCTCCTGCCGACAACTACACAGTGTGTGAAG
GTGACAACGCCACCCTCAGCTGCTTCATCGACGAGCACGTGACCCGCGTGGCCTGGCTGAACCGCTCC
AACATCCTGTATGCCGGCAATGACCGCTGGACCAGCGACCCGCGGGTGCGGCTGCTCATCAACACCCC
CGAGGAGTTCTCCATCCTCATCACCGAGGTGGGGCTCGGCGACGAGGGCCTCTACACCTGCTCCTTCC
AGACCCGCCACCAGCCGTACACCACTCAGGTCTACCTCATTGTCCACGTCCCTGCCCGCATTGTGAAC
ATCTCGTCGCCTGTGACGGTGAATGAGGGGGGCAATGTGAACCTGCTTTGCCTGGCCGTGGGGCGGCC
AGAGCCCACGGTCACCTGGAGACAGCTCCGAGACGGCTTCACCTCGGAGGGAGAGATCCTGGAGATCT
CTGACATCCAGCGGGGCCAGGCCGGGGAGTATGAGTGCGTGACTCACAACGGGGTTAACTCGGCGCCC
GACAGCCGCCGCGTGCTGGTCACAGTCAACTATCCTCCGACCATCACGGACGTGACCAGCGCCCGCAC
CGCGCTGGGCCGGGCCGCCCTCCTGCGCTGCGAAGCCATGGCGGTTCCCCCCGCGGATTTCCAGTGGT
ACAAGGATGACAGACTGCTGAGCAGCGGCACGGCCGAAGGCCTGAAGGTGCAGACGGAGCGCACCCGC
TCGATGCTTCTCTTTGCCAACGTGAGCGCCCGGCATTACGGCAACTATACGTGTCGCGCCGCCAACCG
ACTGGGAGCGTCCAGCGCCTCCATGCGGCTCCTGCGCCCAGGATCCCTGGAGAACTCAGCCCCGAGGC
CCCCAGGGCTCCTGGCCCTCCTCTCCGCCCTGGGCTGGCTGTGGTGGAGAATGCTCGAGGGC

| | | | |
|---|---|---|---|
| NOV11m, CG51027-07 | SEQ ID NO: 306 | 309 aa | MW at 34201.2kD |
| Protein Sequence | | | |

LLSQSLEFNSPADNYTVCEGDNATLSCFIDEHVTRVAWLNRSMILYAGNDRWTSDPRVRLLINTPEEF
SILITEVGLGDEGLYTCSFQTRHQPYTTQVYLIVHVPARIVNISSPVTVNEGGNVNLLCLAVGRPEPT
VTWRQLRDGFTSEGEILEISDIQRGQAGEYECVTHNGVNSAPDSRRVLVTVNYPPTITDVTSARTALG
RAALLRCEAMAVPPADFQWYKDDRLLSSGTAEGLKVQTERTRSMLLFANVSARHYGNYTCRAANRLGA
SSASMRLLRPGSLENSAPRPPGLLALLSALGWLWWRM

| | | |
|---|---|---|
| NOV11n, CG51027-08 | SEQ ID NO: 307 | 1033 bp |
| DNA Sequence | ORF Start: ATG at 17 | ORF Stop: at 1025 |

CACCAAGCTTCCCACCATGCCCCCCCCTGCGCCCGGGGCCCGGCTCCGGCTTCTCGCCGCCGCCGCCC
TGGCCGGCTTGGCCGTCATCAGCCGAGGGCTGCTCTCCCAGAGCCTGGAGTTCAACTCTCCTGCCGAC
AACTACACAGTGTGTGAAGGTGACAACGCCACCCTCAGCTGCTTCATCGACGAGCACGTGACCCGCGT
GGCCTGGCTGAACCGCTCCAACATCCTGTATGCCGGCAATGACCGCTGGACCAGCGACCCGCGGGTGC
GGCTGCTCATCAACACCCCCGAGGAGTTCTCCATCCTCATCACCGAGGTGGGGCTCGGCGACGAGGGC
CTCTACACCTGCTCCTTCCAGACCCGCCACCAGCCGTACACCACTCAGGTCTACCTCATTGTCCACGT
CCCTGCCCGCATTGTGAACATCTCGTCGCCTGTGACGGTGAATGAGGGGGGCAATGTGAACCTGCTTT
GCCTGGCCGTGGGGCGGCCAGAGCCCACGGTCACCTGGAGACAGCTCCGAGACGGCTTCACCTCGGAG
GGAGAGATCCTGGAGATCTCTGACATCCAGCGGGGCCAGGCCGGGGAGTATGAGTGCGTGACTCACAA
CGGGGTTAACTCGGCGCCCGACAGCCGCCGCGTGCTGGTCACAGTCAACTATCCTCCGACCATCACGG
ACGTGACCAGCGCCCGCACCGCGCTGGGCCGGGCCGCCCTCCTGCGCTGCGAAGCCATGGCGGTTCCC
CCCGCGGATTTCCAGTGGTACAAGGATGACAGACTGCTGAGCAGCGGCACGGCCGAAGGCCTGAAGGT
GCAGACGGAGCGCACCCGCTCGATGCTTCTCTTTGCCAACGTGAGCGCCCGGCATTACGGCAACTATA
CGTGTCGCGCCGCCAACCGACTGGGAGCGTCCAGCGCCTCCATGCGGCTCCTGCGCCCAGGATCCCTG
GAGAACTCAGCCCCGAGGCCCCCAGGGCTCCTGGCCCTCCTCTCCGCCCTGGGCTGGCTGTGGTGGAG
AATGCTCGAGGGC

| | | | |
|---|---|---|---|
| NOV11n, CG51027-08 | SEQ ID NO: 308 | 336 aa | MW at 36794.3kD |
| Protein Sequence | | | |

MPPPAPGARLRLLAAAALAGLAVISRGLLSQSLEFNSPADNYTVCEGDNATLSCFIDEHVTRVAWLNR
SNILYAGNDRWTSDPRVRLLINTPEEFSILITEVGLGDEGLYTCSFQTRHQPYTTQVYLIVHVPARIV
NISSPVTVNEGGNVNLLCLAVGRPEPTVTWRQLRDGFTSEGEILEISDIQRGQAGEYECVTHNGVNSA
PDSRRVLVTVNYPPTITDVTSARTALGRAALLRCEAMAVPPADFQWYKDDRLLSSGTAEGLKVQTERT
RSMLLFANVSARHYGNYTCRAANPLGASSASMRLLRPGSLENSAPRPPGLLALLSALGWLWWPM

| | | |
|---|---|---|
| NOV11o, CG51027-10 | SEQ ID NO: 309 | 1035 bp |
| DNA Sequence | ORF Start: ATG at 16 | ORF Stop: TAG at 1024 |

CACCGCGGCCGCACCATGCCCCCCCCTGCGCCCGGGGCCCGGCTCCGGCTTCTCGCCGCCGCCGCCCT
GGCCGGCTTGGCCGTCATCAGCCGAGGGCTGCTCTCCCAGAGCCTGGAGTTCAACTCTCCTGCCGACA
ACTACACAGTGTGTGAAGGTGACAACGCCACCCTCAGCTGCTTCATCGACGAGCACGTGACCCGCGTG
GCCTGGCTGAACCGCTCCAACATCCTGTATGCCGGCAATGACCGCTGGACCAGCGACCCGCGGGTGCG
GCTGCTCATCAACACCCCCGAGGAGTTCTCCATCCTCATCACCGAGGTGGGGCTCGGCGACGAGGGCC
TCTACACCTGCTCCTTCCAGACCCGCCACCAGCCGTACACCACTCAGGTCTACCTCATTGTCCACGTC
CCTGCCCGCATTGTGAACATCTCGTCGCCTGTGACGGTGAATGAGGGGGGCAATGTGAACCTGCTTTG
CCTGGCCGTGGGGCGGCCAGAGCCCACGGTCACCTGGAGACAGCTCCGAGACGGCTTCACCTCGGAGG
GAGAGATCCTGGAGATCTCTGACATCCAGCGGGGCCAGGCCGGGGAGTATGAGTGCGTGACTCACAAC
GGGGTTAACTCGGCGCCCGACAGCCGCCGCGTGCTGGTCACAGTCAACTATCCTCCGACCATCACGGA
CGTGACCAGCGCCCGCACCGCGCTGGGCCGGGCCGCCCTCCTGCGCTGCGAAGCCATGGCGGTTCCCC
CCGCGGATTTCCAGTGGTACAAGGATGACAGACTGCTGAGCAGCGGCACGGCCGAAGGCCTGAAGGTG
CAGACGGAGCGCACCCGCTCGATGCTTCTCTTTGCCAACGTGAGCGCCCGGCATTACGGCAACTATAC
GTGTCGCGCCGCCAACCGACTGGGAGCGTCCAGCGCCTCCATGCGGCTCCTGCGCCCAGGATCCCTGG
AGAACTCAGCCCCGAGGCCCCCAGGGCTCCTGGCCCTCCTCTCCGCCCTGGGCTGGCTGTGGTGGAGA
ATGTAGGTCGACGGC

| | | | |
|---|---|---|---|
| NOV11o, CG51027-10 | SEQ ID NO: 310 | 336 aa | MW at 36794.3kD |
| Protein Sequence | | | |

MPPPAPGARLRLLAAAALAGLAVISRGLLSQSLEFNSPADNYTVCEGDNATLSCFIDEHVTRVAWLNR
SNILYAGNDRWTSDPRVRLLINTPEEFSILITEVGLGDEGLYTCSFQTRHQPYTTQVYLIVHVPARIV
NISSPVTVNEGGNVNLLCLAVGRPEPTVTWRQLRDGFTSEGEILEISDIQRGQAGEYECVTHNGVNSA
PDSRRVLVTVNYPPTITDVTSARTALGRAALLRCEAMAVPPALFQWYKDDRLLSSGTAEGLKVQTERT
RSMLLFANVSARHYGNYTCRAANRLGASSASMRLLRPGSLENSAPRPPGLLALLSALGWLWWRM

| | | |
|---|---|---|
| NOV11p, CG51027-11 | SEQ ID NO: 311 | 976 bp |
| DNA Sequence | ORF Start: at 11 | ORF Stop: at 971 |

CACCTCGCGACTGGCCGGCTTGGCCGTCATCAGCCGAGGGCTGCTCTCCCAGAGCCTGGAGTTCAACT
CTCCTGCCGACAACTACACAGTGTGTGAAGGTGACAACGCCACCCTCAGCTGCTTCATCGACGAGCAC
GTGACCCGCGTGGCCTGGCTGAACCGCTCCAACATCCTGTATGCCGGCAATGACCGCTGGACCAGCGA
CCCGCGGGTGCGGCTGCTCATCAACACCCCCGAGGAGTTCTCCATCCTCATCACCGAGGTGGGGCTCG
GCGACGAGGGCCTCTACACCTGCTCCTTCCAGACCCGCCACCAGCCGTACACCACTCAGGTCTACCTC
ATTGTCCACGTCCCTGCCCGCATTGTGAACATCTCGTCGCCTGTGACGGTGAATGAGGGGGGCAATGT
GAACCTGCTTTGCCTGGCCGTGGGGCGGCCAGAGCCCACGGTCACCTGGAGACAGCTCCGAGACGGCT
TCACCTCGGAGGGAGAGATCCTGGAGATCTCTGACATCCAGCGGGGCCAGGCCGGGGAGTATGAGTGC
GTGACTCACAACGGGGTTAACTCGGCGCCCGACAGCCGCCGCGTGCTGGTCACAGTCAACTATCCTCC
GACCATCACGGACGTGACCAGCGCCCGCACCGCGCTGGGCCGGGCCGCCCTCCTGCGCTGCGAAGCCA
TGGCGGTTCCCCCCGCGGATTTCCAGTGGTACAAGGATGACAGACTGCTGAGCAGCGGCACGGCCGAA
GGCCTGAAGGTGCAGACGGAGCGCACCCGCTCGATGCTTCTCTTTGCCAACGTGAGCGCCCGGCATTA
CGGCAACTATACGTGTCGCGCCGCCAACCGACTGGGAGCGTCCAGCGCCTCCATGCGGCTCCTGCGCC
CAGGATCCCTGGAGAACTCAGCCCCGAGGCCCCCAGGGCTCCTGGCCCTCCTCTCCGCCCTGGGCTGG
CTGTGGTGGAGAATGGTCGACGGC

| | | | |
|---|---|---|---|
| NOV11p, CG51027-11 | SEQ ID NO: 312 | 320 aa | MW at 35238.5kD |
| Protein Sequence | | | |

LAGLAVISRGLLSQSLEFNSPADNYTVCEGDNATLSCFIDEHVTRVAWLNRSNILYAGNDRWTSDPRV
RLLINTPEEFSILITEVGLGDEGLYTCSFQTRHQPYTTQVYLIVHVPARIVNISSPVTVNEGGNVNLL
CLAVGRPEPTVTWRQLRDGFTSEGEILEISDIQRGQAGEYECVTHNGVNSAPDSRRVLVTVNYPPTIT
DVTSARTALGRAALLRCEAMAVPPADFQWYKDDRLLSSGTAEGLKVQTERTRSMLLFANVSARHYGNY
TCRAANRLGASSASMRLLRPGSLENSAPRPPGLLALLSALGWLWWRMV

| | | |
|---|---|---|
| NOV11q, CG51027-12 | SEQ ID NO: 313 | 1030 bp |
| DNA Sequence | ORF Start: ATG at 14 | ORF Stop: at 1022 |

CACCTCGCGAACCATGCCCCCCCCTGCGCCCGGGGCCCGGCTCCGGCTTCTCGCCGCCGCCGCCCTGG
CCGGCTTGGCCGTCATCAGCCGAGGGCTGCTCTCCCAGAGCCTGGAGTTCAACTCTCCTGCCGACAAC
TACACAGTGTGTGAAGGTGACAACGCCACCCTCAGCTGCTTCATCGACGAGCACGTGACCCGCGTGGC
CTGGCTGAACCGCTCCAACATCCTGTATGCCGGCAATGACCGCTGGACCAGCGACCCGCGGGTGCGGC
TGCTCATCAACACCCCCGAGGAGTTCTCCATCCTCATCACCGAGGTGGGGCTCGGCGACGAGGGCCTC
TACACCTGCTCCTTCCAGACCCGCCACCAGCCGTACACCACTCAGGTCTACCTCATTGTCCACGTCCC
TGCCCGCATTGTGAACATCTCGTCGCCTGTGACGGTGAATGAGGGGGGCAATGTGAACCTGCTTTGCC
TGGCCGTGGGGCGGCCAGAGCCCACGGTCACCTGGAGACAGCTCCGAGACGGCTTCACCTCGGAGGGA
GAGATCCTGGAGATCTCTGACATCCAGCGGGGCCAGGCCGGGGAGTATGAGTGCGTGACTCACAACGG
GGTTAACTCGGCGCCCGACAGCCGCCGCGTGCTGGTCACAGTCAACTATCCTCCGACCATCACGGACG
TGACCAGCGCCCGCACCGCGCTGGGCCGGGCCGCCCTCCTGCGCTGCGAAGCCATGGCGGTTCCCCCC
GCGGATTTCCAGTGGTACAAGGATGACAGACTGCTGAGCAGCGGCACGGCCGAAGGCCTGAAGGTGCA
GACGGAGCGCACCCGCTCGATGCTTCTCTTTGCCAACGTGAGCGCCCGGCATTACGGCAACTATACGT
GTCGCGCCGCCAACCGACTGGGAGCGTCCAGCGCCTCCATGCGGCTCCTGCGCCCAGGATCCCTGGAG
AACTCAGCCCCGAGGCCCCCAGGGCTCCTGGCCCTCCTCTCCGCCCTGGGCTGGCTGTGGTGGAGAAT
GGTCGACGGC

| | | | |
|---|---|---|---|
| NOV11q, CG51027-12 | SEQ ID NO: 314 | 336 aa | MW at 36794.3kD |
| Protein Sequence | | | |

MPPPAPGARLRLLAAAALAGLAVISRGLLSQSLEFNSPADNYTVCEGDNATLSCFIDEHVTRVAWLNR
SNILYAGNDRWTSDPRVRLLINTPEEFSILITEVGLGDEGLYTCSFQTRHQPYTTQVYLIVHVPARIV
NISSPVTVNEGGNVNLLCLAVGRPEPTVTWRQLRDGFTSEGEILEISDIQRGQAGEYECVTHNGVNSA
PDSRRVLVTVNYPPTITDVTSARTALGRAALLRCEAMAVPPADFQWYKDDRLLSSGTAEGLKVQTERT
RSMLLFANVSARHYGNYTCRAANRLGASSASMRLLRPGSLENSAPRPPGLLALLSALGWLWWRM

| | | |
|---|---|---|
| NOV11r, SNP13376340 | SEQ ID NO: 315 | 1271 bp |
| of CG51027-06, | ORF Start: ATG at 48 | ORF Stop: TAG at 1056 |
| DNA Sequence | SNP Pos: 888 | SNP Change: G to A |

GCGCAGTGTGCTGGAATTCGCCCTTCCTCCGTGCCGCCTCTGCCGCGATGCCCCCCCCTGCGCCCGGG
GCCCGGCTCCGGCTTCTCGCCGCCGCCGCCCTGGCCGGCTTGGCCGTCATCAGCCGAGGGCTGCTCTC
CCAGAGCCTGGAGTTCAACTCTCCTGCCGACAACTACACAGTGTGTGAAGGTGACAACGCCACCCTCA
GCTGCTTCATCGACGAGCACGTGACCCGCGTGGCCTGGCTGAACCGCTCCAACATCCTGTATGCCGGC
AATGACCGCTGGACCAGCGACCCGCGGGTGCGGCTGCTCATCAACACCCCCGAGGAGTTCTCCATCCT
CATCACCGAGGTGGGGCTCGGCGACGAGGGCCTCTACACCTGCTCCTTCCAGACCCGCCACCAGCCGT
ACACCACTCAGGTCTACCTCATTGTCCACGTCCCTGCCCGCATTGTGAACATCTCGTCGCCTGTGATG
GTGAATGAGGGGGGCAATGTGAACCTGCTTTGCCTGGCCGTGGGGCGGCCAGAGCCCACGGTCACCTG
GAGACAGCTCCGAGACGGCTTCACCTCGGAGGGAGAGATCCTGGAGATCTCTGACATCCAGCGGGGCC
AGGCCGGGGAGTATGAGTGCGTGACTCACAACGGGGTTAACTCGGCGCCCGACAGCCGCCGCGTGCTG
GTCACAGTCAACTATCCTCCGACCATCACGGACGTGACCAGCGCCCGCACCGCGCTGGGCCGGGCCGC
CCTCCTGCGCTGCGAAGCCATGGCGGTTCCCCCCGCGGATTTCCAGTGGTATAAGGATGACAGACTGC
TGAGCAGCGGCACGGCCGAAGGCCTGAAGGTGCAGATGGAGCGCACCCGCTCGATGCTTCTCTTTGCC
AACATGAGCGCCCGGCATTACGGCAACTATACGTGTTGCGCCGCCAACCGACTGGGAGCGTCCAGCGC
CTCCATGCGGCTCCTGTGCCCAGGATCCCTGGAGAACTCAGCCCCGAGGCCCCCAGGGCTCCTGGCCC
TCCTCTCCGCCCTGGGCTGGCTGTGGTGGAGAATGTAGGCGCAACCCAGTGGAGCTCGCCTCCCCCTG
CAGGGGGCCTCAGGCCAAGAGTGAGAGAAAAGGGGGAGCAAGAGCCCTGGGTCTCGTGGGGGCAGAAG
GGCTCTCGGCCACCAAGGAAGAAGAGAAGAGGAGAAAGAGGAGGAGGCAGAGGAAGAAAGATCTTTAG
AGAACCCATCACTGTGAGGGATAACGCAAAATTATGCATCTTTCTAC

| | | | |
|---|---|---|---|
| NOV11r, SNP13376340 | SEQ ID NO: 316 | 336 aa | MW at 36780.5kD |
| of CG51027-06, | SNP Pos: 281 | | SNP Change: |
| Protein Sequence | | | Val to Met |

MPPPAPGARLRLLAAAALAGLAVISRGLLSQSLEFNSPADNYTVCEGDNATLSCFIDEHVTRVAWLNR
SNILYAGNDRWTSDPRVRLLINTPEEFSILITEVGLGDEGLYTCSFQTRHQPYTTQVYLIVHVPARTV
NISSPVMVNEGGNVNLLCLAVGRPEPTVTWRQLRDGFTSEGEILEISDIQRGQAGEYECVTHNGVNSA
PDSRRVLVTVNYPPTITDVTSARTALGRAALLRCEAMAVPPADFQWYKDDRLLSSGTAEGLKVQMERT
RSMLLFANMSARHYGNYTCCAANPLGASSASMRLLCPGSLENSAPRPPGLLALLSALGWLWWRM

| | | |
|---|---|---|
| NOV11s, SNP13376341 | SEQ ID NO: 317 | 1271 bp |
| of CG51027-06, | ORF Start: ATG at 48 | ORF Stop: TAG at 1056 |
| DNA Sequence | SNP Pos: 941 | SNP Change: A to G |

GCGCAGTGTGCTGGAATTCGCCCTTCCTCCGTGCCGCCTCTGCCGCGATGCCCCCCCCTGCGCCCGGG
GCCCGGCTCCGGCTTCTCGCCGCCGCCGCCCTGGCCGGCTTGGCCGTCATCAGCCGAGGGCTGCTCTC
CCAGAGCCTGGAGTTCAACTCTCCTGCCGACAACTACACAGTGTGTGAAGGTGACAACGCCACCCTCA
GCTGCTTCATCGACGAGCACGTGACCCGCGTGGCCTGGCTGAACCGCTCCAACATCCTGTATGCCGGC
AATGACCGCTGGACCAGCGACCCGCGGGTGCGGCTGCTCATCAACACCCCCGAGGAGTTCTCCATCCT
CATCACCGAGGTGGGGCTCGGCGACGAGGGCCTCTACACCTGCTCCTTCCAGACCCGCCACCAGCCGT
ACACCACTCAGGTCTACCTCATTGTCCACGTCCCTGCCCGCATTGTGAACATCTCGTCGCCTGTGATG
GTGAATGAGGGGGGCAATGTGAACCTGCTTTGCCTGGCCGTGGGGCGGCCAGAGCCCACGGTCACCTG
GAGACAGCTCCGAGACGGCTTCACCTCGGAGGGAGAGATCCTGGAGATCTCTGACATCCAGCGGGGCC
AGGCCGGGGAGTATGAGTGCGTGACTCACAACGGGGTTAACTCGGCGCCCGACAGCCGCCGCGTGCTG
GTCACAGTCAACTATCCTCCGACCATCACGGACGTGACCAGCGCCCGCACCGCGCTGGGCCGGGCCGC
CCTCCTGCGCTGCGAAGCCATGGCGGTTCCCCCCGCGGATTTCCAGTGGTATAAGGATGACAGACTGC
TGAGCAGCGGCACGGCCGAAGGCCTGAAGGTGCAGATGGAGCGCACCCGCTCGATGCTTCTCTTTGCC
AACGTGAGCGCCCGGCATTACGGCAACTATACGTGTTGCGCCGCCAACCGACTGGGGGCGTCCAGCGC
CTCCATGCGGCTCCTGTGCCCAGGATCCCTGGAGAACTCAGCCCCGAGGCCCCCAGGGCTCCTGGCCC
TCCTCTCCGCCCTGGGCTGGCTGTGGTGGAGAATGTAGGCGCAACCCAGTGGAGCTCGCCTCCCCCTG
CAGGGGGCCTCAGGCCAAGAGTGAGAGAAAAGGGGGAGCAAGAGCCCTGGGTCTCGTGGGGGCAGAAG
GGCTCTCGGCCACCAAGGAAGAAGAGAAGAGGAGAAAGAGGAGGAGGCAGAGGAAGAAAGATCTTTAG
AGAACCCATCACTGTGAGGGATAACGCAAAATTATGCATCTTTCTAC

| | | | |
|---|---|---|---|
| NOV11s, SNP13376341 | SEQ ID NO: 318 | 336 aa | MW at 36748.4kD |
| of CG51027-06, | SNP Pos: 298 | | SNP Change: |
| Protein Sequence | | | Gly to Gly |

MPPPAPGARLRLLAAAALAGLAVISRGLLSQSLEFNSPADNYTVCEGDNATLSCFIDEHVTRVAWLNR
SNILYAGNDRWTSDPRVRLLINTPEEFSILITEVGLGDEGLYTCSFQTRHQPYTTQVYLIVHVPARIV
NISSPVMVNEGGNVNLLCLAVGRPEPTVTWRQLRDGFTSEGEILEISDIQRGQAGEYECVTHNGVNSA
PDSRRVLVTVNYPPTITDVTSARTALGRAALLRCEAMAVPPADFQWYKDDRLLSSGTAEGLKVQMERT
RSMLLFAIVSARHYGNYTCCAANPLGASSASMRLLCPGSLENSAPRPPGLEALLSALGWLWWRM

A ClustalW comparison of the above protein sequences yields the following sequence alignment shown in Table 11B.

TABLE 11B
Comparison of the NOV11 protein sequences.

NOV11a -----MPPPAPGARLRLLAAAALAGLAVISRGLLSQSLEFNSPADNYTVCEGDNATLSCF
NOV11b TKLPTMPPPAPGARLRLLAAAALAGLAVISRGLLSQSLEFNSPADNYTVCEGDNATLSCF
NOV11c -------------------TKLLAGLAVISRGLLSQSLEFNSPADNYTVCEGDNATLSCF
NOV11d ------------------------------------------------TKLGDNATLSCF
NOV11e -----------------------------TKLLLSQSLEFNSPADNYTVCEGDNATLSCF
NOV11f ----------------------LAGLAVISRGLLSQSLEFNSPADNYTVCEGDNATLSCF
NOV11g -------------------TSRLAGLAVISRGLLSQSLEFNSPADNYTVCEGDNATLSCF
NOV11h ----------------------LAGLAVISRGLLSQSLEFNSPADNYTVCEGDNATLSCF
NOV11i ----------------------------------SQSLEFNSPADNYTVCEGDNATLSCF
NOV11j ----------------------LAGLAVISRGLLSQSLEFNSPADNYTVCEGDNATLSCF
NOV11k -------------------------LAVISRGLLSQSLEFNSPADNYTVCEGDNATLSCF
NOV11l -----MPPAAPGARLRLLAAAALAGLAVISRGLLSQRLEFNSPADNYTVCEGDNATLSCF
NOV11m --------------------------------LLSQSLEFNSPADNYTVCEGDNATLSCF
NOV11n -----MPPPAPGARLRLLAAAALAGLAVISRGLLSQSLEFNSPADNYTVCEGDNATLSCF
NOV11o -----MPPPAPGARLRLLAAAALAGLAVISRGLLSQSLEFNSPADNYTVCEGDNATLSCF
NOV11p ----------------------LAGLAVISRGLLSQSLEFNSPADNYTVCEGDNATLSCF
NOV11q -----MPPPAPGARLRLLAAAALAGLAVISRGLLSQSLEFNSPADNYTVCEGDNATLSCF
NOV11a IDEHVTRVAWLNRSNILYAGNDRWTSDPRVRLLINTPEEFSILITEVGLGDEGLYTCSFQ
NOV11b IDEHVTRVAWLNRSNILYAGNDRWTSDPRVRLLINTPEEFSILITEVGLGDEGLYTCSFQ
NOV11c IDEHVTRVAWLNRSNILYAGNDRWTSDPRVRLLINTPEEFSILITEVGLGDEGLYTCSFQ
NOV11d IDEHVTRVAWLNRSNTLYAGNDRWTSDPRVRLLINTPEEFSILITEVGLGDEGLYTCSFQ
NOV11e IDEHVTRVAWLNRSNILYAGNDRWTSDPRVRLLINTPEEFSILITEVGLGDEGLYTCSFQ
NOV11f IDEHVTRVAWLNRSNTLYAGNDRWTSDPRVRLLINTPEEFSILITEVGLGDEGLYTCSFQ
NOV11g IDEBVTRVAWLNRSNILYAGNDRWTSDPRVRLLINTPEEFSILITEVGLGDEGLYTCSFQ
NOV11h IDEHVTRVAWLNRSNILYAGNDRWTSDPRVRLLINTPEEFSILITEVGLGDEGLYTCSFQ
NOV11i IDEHVTRVAWLNRSNILYAGNDRWTSDPRVRLLINTPEEFSILITEVGLGDEGLYTCSFQ
NOV11j IDEHVTRVAWLNRSNILYAGNDRWTSDPRVWLLINTPEEFSILITEVGLGDEGLYTCSFQ
NOV11k IDENVTRVAWLNRSNILYAGNDRWTSDPRVRLLINTPEEFSILITEVGLGDEGLYTCSFQ
NOV11l MDEHVTRVAWLNRSNILYAGNDRRTRDPRVRLLINTPEEFSILVTEVGLGDEGLYTCSFQ
NOV11m IDENVTRVAWLNRSNILYAGNDRWTSDPRVRLLINTPEEFSILITEVGLGDEGLYTCSFQ
NOV11n IDEHVTRVAWLNRSNILYAGNDRWTSDPRVRLLINTPEEFSILITEVGLGDEGLYTCSFQ
NOV11o IDEHVTRVAWLNRSNILYAGNDRWTSDPRVRLLINTPEEFSILITEVGLGDEGLYTCSFQ
NOV11p IDEHVTRVAWLNRSNILYAGNDRWTSDPRVRLLINTPEEFSILITEVGLGDEGLYTCSFQ
NOV11q IDEHVTRVAWLNRSNILYAGNDRWTSDPRVRLLINTPEEFSILITEVGLGDEGLYTCSFQ
NOV11a TRHQPYTTQVYLIVHVPARIVNISSPVMVNEGGNVNLLCLAVGRPEPTVTWRQLRDGFTS
NOV11b TRHQPYTTQVYLIVHVPARIVNISSPVTVNEGGNVNLLCLAVGRPEPTVTWRQLRDGFTS
NOV11c TRHQPYTTQVYLIVHVPARIVNISSPVTVNEGGNVNLLCLAVGRPEPTVTWRQLRDGFTS
NOV11d TRHQPYTTQVYLIVHVPARIVNISSPVTVNEGGNVNLLCLAVGRPEPTVTWRQLRDGFTS
NOV11e TRHQPYTTQVYLIVHVPARIVNISSPVTVNEGGNVNLLCLAVGRPEPTVTWRQLRDGFTS
NOV11f TRHQPYTTQVYLIVHVPARIVNISSPVTVNEGGNVNLLCLAVGRPEPTVTWRQLRDGFTS
NOV11g TRHQPYTTQVYLIVHVPARIVNISSPVTVNEGGNVNLLCLAVGRPEPTVTWRQLRDGFTS
NOV11h TRHQPYTTQVYLIVHVPARIVNISSPVRTNEGGNVNLLCLAVGRPEPTVTWRQLRDGFTS
NOV11i TRHQPYTTQVYLIVHVPARIVNISSPVTVNEGGNVNLLCLAVGRPEPTVTWRQLRDGFTS
NOV11j TRHQPYTTQVYLIVHVPARIVNISSPVMVNEGGNVNLLCLAVGRPEPTVTWRQLRDGFTS
NOV11k TRHQPYTTQVYLIVHVPARIVNISSPVTVNEGGNVNLLCLAVGRPEPTVTWRQLRDGFTS
NOV11l TRHQPYTTQVYLIVHVPARVVNISSPVMVNEGGNVNLLCLAVGRPEPTVTWRQLRDGFTS
NOV11m TRHQPYTTQVYLIVHVPARIVNISSPVTVNEGGNVNLLCLAVGRPEPTVTWRQLRDGFTS
NOV11n TRHQPYTTQVYLIVHVPARIVNISSPVTVNEGGNVNLLCIAVGRPEPTVTWRQLRDGFTS
NOV11o TRHQPYTTQVYLIVHVPARIVNISSPVTVNEGGNVNLLCLAVGRPEPTVTWRQLRDGFTS
NOV11p TRHQPYTTQVYLIVHVPARIVNISSPVTVNEGGNVNLLCLAVGRPEPTVTWRQLRDGFTS
NOV11q TRHQPYTTQVYLIVHVPARIVNISSPVTVNEGGNVNLLCLAVGRPEPTVTWRQLRDGFTS
NOV11a EGEILEISDIQRGQAGEYECVTHNGVNSAPDSRRVLVTVNYPPTITDVTSARTALGRAAL
NOV11b EGEILEISDIQRGQAGEYECVTHNGVNSAPDSRRVLVTVNYPPTITDVTSARTALGRAAL
NOV11c EGEILEISDIQRGQAGEYECVTHNGVNSAPDSRRVLVTVNYPPTITDVTSARTALGRAAL
NOV11d EGEILEISDIQRGQAGEYECVTHNGVNSAPDSRRVLVTVNYPPTITDVTSARTALGRAAL
NOV11e EGEILEISDIQRGQAGEYECVTHNGVNSAPDSRRVLVTVNYPPTITDVTSARTALGRAAL
NOV11f EGEILEISDIQRGQAGEYECVTHNGVNSAPDSRRVLVTVNYPPTITDVTSARTALGRAAL
NOV11g EGEILEISDIQRGQAGEYECVTHNGVNSAPDSRRVLVTVNYPPTITDVTSARTALGRAAL
NOV11h EGEILEISDIQRGQAGEYECVTHNGVNSAPDSRRVLVTVNYPPTITDVTSARTALGRTAL
NOV11i EGEILEISDIQRGQAGEYECVTHNGVNSAPDSRRVLVTVNYPPTITDVTSARTALGRAAL
NOV11j EGEILEISDIQRGQAGEYECVTHNGVNSAPDSRRVLVTVNYPPTITDVTSARTALGRAAL
NOV11k EGEILEISDIQRGQAGEYECVTHNGVNSAPDSRRVLVTVNYPPTITDVTSARTALGRAAL
NOV11l EGEILEISDILRGQAGEYECVTHNGVNSAPDSRRVLVTVNYPPTITDVTSARTALGPGRL
NOV11m EGEILEISDIQRGQAGEYECVTHNGVNSAPDSRRVLVTVNYPPTITDVTSARTALGRAAL
NOV11n EGEILEISDIQRGQAGEYECVTHNGVNSAPDSRRVLVTVNYPPTITDVTSARTALGRAAL
NOV11o EGEILEISDIQRGQAGEYECVTHNGVNSAPDSRRVLVTVNYPPTITDVTSARTALGRAAL
NOV11p EGEILEISDIQRGQAGEYECVTHNGVNSAPDSRRVLVTVNYPPTITDVTSARTALGRAAL
NOV11q EGEILEISDIQRGQAGEYECVTHNGVNSAPDSRRVLVTVNYPPTITDVTSARTALGRAAL
NOV11a LRCEAMAVPPADFQWYKDDRLLSSGTAEGLKVQMERTRSMLLFANVSARHYGNYTCCAAN
NOV11b LRCEAMAVPPADFQWYKDDRLLSSGTAEGLKVQTERTRSMLLFANVSARHYGNYTCRAAN
NOV11c LRCEAMAVPPADFQWYKDDRLLSSGTAEGLKVQTERTRSMLLFANVSARHYGNYTCRAAN
NOV11d LRCEAMAVPPADFQWYKDDRLLSSGTAEGLKVQTERTRSMLLFANVSARHYGNYTCRALE
NOV11e LRCEAMAVPPADFQWYKDDRLLSSGTAEGLKVQTERTRSMLLFANVSARHYGNYTCRAAN
NOV11f LRCEAMAVPPADFQWYKDDRLLSSGTAEGLKVQTERTRSMLLFANVSARHYGNYTCRAAN
NOV11g LRCEAMAVPPADFQWYKDDRLLSSGTAEGLKVQTERTRSMLLFANVSARHYGNYTCRAAN
NOV11h LRCEAMAVPPADFQWYKDDRLLSSGTAEGLKVQTERTRSMLLFANVSARHYGNYTCRAAN
NOV11i LRCEAMAVPPADFQWYKDDRLLSSGTAEGLKVQTERTRSMLLFANVSARHYGNYTCRAAN
NOV11j LRCEAMAVPPADFQWYKDDRLLSSGTAEGLKVQMERTRSMLLFANVSARHYGNYTCRAAN
NOV11k LRCEAMAVPPADFQWYKDDRLLSSGTAEGLKVQTERTRSMLLFANVSARHYGNYTCRAAN
NOV11l LRCEAMAVSPADFQWYKDDRLLSSGTAEGLKVQMERTRSMLLFANMSARHYGNYTCCAAN
NOV11m LRCEAMAVPPADFQWYKDDRLLSSGTAEGLKVQTERTRSMLLFANVSARHYGNYTCRAAN
NOV11n LRCEAMAVPPADFQWYKDDRLLSSGTAEGLKVQTERTRSMLLFANVSARHYGNYTCPAAN
NOV11o LRCEAMAVPPADFQWYKDDRLLSSGTAEGLKVQTERTRSMLLFANVSARHYGNYTCRAAN
NOV11p LRCEAMAVPPADFQWYKDDRLLSSGTAEGLKVQTERTRSMLLFANVSARHYGNYTCRAAN
NOV11q LRCEAMAVPPADFQWYKDDRLLSSGTAEGLKVQTERTRSMLLFANVSARHYGNYTCRAAN
NOV11a RLGASSASMRLLCPGSLENSAPRPPGLLALLSALGWLWWRM---
NOV11b RLGASSASMPLLRPGSLENSAPRPPGLLALLSALGWLWWRMLEG
NOV11c RLGASSASMRLLRPGSLENSAPRPPGLLALLSALGWLWWRMLEG
NOV11d G-------------------------------------------
NOV11e RLGASSASMRLLRPGSLENSAPRPPGLLALLSALGWLWWRMLEG
NOV11f RLGASSASMRLLRPGSLENSAPRPPGLLALLSALGWLWWPM---
NOV11g RLGASSASMRLLRPGSLENSAPRPPGLLALLSALGWLWWRMVDG
NOV11h RLGASSASMRLLRPGSLENSAPRPPGLLALLSALGWLWWRM---
NOV11i RLGASSASMRLLRPGSLENSAPRPPGLLALLSALGWLWWRM---
NOV11j RLGASSASMRLLRPGSLENSAPRPPGLLALLSALGWLWWRM---
NOV11k RLGASSASMRLLRPGSLENSAPRPPGLLALLSAL----------
NOV11l RLGASSASMRLLCPGSLENSAPRPPGPLALLSALGWLWWRM---
NOV11m RLGASSASMRLLRPGSLENSAPRPPGLLALLSALGWLWWRM---
NOV11n RLGASSASMRLLRPGSLENSAPRPPGLLALLSALGWLWWRM---
NOV11o RLGASSASMRLLRPGSLENSAPRPPGLLALLSALGWLWWRM---
NOV11p RLGASSASMRLLRPGSLENSAPRPPGLLALLSALGWLWWPMV--
NOV11q RLGASSASMRLLRPGSLENSAPRPPGLLALLSALGWLWWRM---
NOV11a (SEQ ID NO: 282)
NOV11b (SEQ ID NO: 284)
NOV11c (SEQ ID NO: 286)
NOV11d (SEQ ID NO: 288)
NOV11e (SEQ ID NO: 290)
NOV11f (SEQ ID NO: 292)
NOV11g (SEQ ID NO: 294)
NOV11h (SEQ ID NO: 296)
NOV11i (SEQ ID NO: 298)
NOV11j (SEQ ID NO: 300)
NOV11k (SEQ ID NO: 302)
NOV11l (SEQ ID NO: 304)
NOV11m (SEQ ID NO: 306)
NOV11n (SEQ ID NO: 308)
NOV11o (SEQ ID NO: 310)
NOV11p (SEQ ID NO: 312)
NOV11q (SEQ ID NO: 314)

Further analysis of the NOV11a protein yielded the following properties shown in Table 11C.

TABLE 11C
Protein Sequence Properties NOV11a

| | |
|---|---|
| SignalP analysis: | Cleavage site between residues 28 and 29 |
| PSORT II analysis: | |

| PSG: | a new signal peptide prediction method | |
|---|---|---|
| | N-region: | length 11; pos. chg 2; neg. chg 0 |
| | H-region: | length 14; peak value 8.95 |
| | PSG score: | 4.55 |

| | |
|---|---|
| GvH: | von Heijne's method for signal seq. recognition |
| | GvH score (threshold: −2.1): −2.40 |
| | possible cleavage site: between 22 and 23 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 1 | | |
| | Number of TMS(s) for threshold 0.5: 1 | | |
| | INTEGRAL | Likelihood = | Transmembrane 12-28 |
| | | −2.92 | |

| | |
|---|---|
| PERIPHERAL | Likelihood = 2.49 (at 128) |

| | |
|---|---|
| | ALOM score: −2.92 (number of TMSs: 1) |
| MTOP: | Prediction of membrane topology (Hartmann et al.) |
| | Center position for calculation: 19 |
| | Charge difference: −4.0 C(−1.0)-N( 3.0) |
| | N >= C: N-terminal side will be inside |

>>> membrane topology: type 2 (cytoplasmic tail 1 to 12)

| MITDISC: | discrimination of mitochondrial targeting seq | | | |
|---|---|---|---|---|
| | R content: | 3 | Hyd Moment (75): | 1.42 |
| | Hyd Moment (95): | 2.81 | G content: | 3 |
| | D/E content: | 1 | S/T content: | 3 |

| | |
|---|---|
| | Score: −3.82 |
| Gavel: | prediction of cleavage sites for mitochondrial preseq |
| | R-2 motif at 36 SRG\|LL |

NUCDISC: discrimination of nuclear localization signals

| | |
|---|---|
| | pat4: none |
| | pat7: none |
| | bipartite: none |
| | content of basic residues: 8.6% |
| | NLS Score: −0.47 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 76.7 |
| COIL: | Lupas's algorithm to detect coiled-coil regions |
| | total: 0 residues |

Final Results (k = 9/23):

```
34.8%: cytoplasmic
30.4%: mitochondrial
13.0%: Golgi
8.7%: endoplasmic reticulum
  4.3%: extracellular, including cell wall
  4.3%: nuclear
  4.3%: vesicles of secretory system
```

>> prediction for CG51027-06 is cyt (k = 23)

A search of the NOV11a protein against the Geneseq database, a proprietary database that contains sequences published in patents and patent publication, yielded several homologous proteins shown in Table 11D.

TABLE 11D
Geneseq Results for NOV11a

| | | NOV11a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE29924 | Human LP289 protein - Homo sapiens, | 1 . . . 336 | 332/336 (98%) | 0.0 |
| | 336 aa. [WO200274906-A2, 26-SEP-2002] | 1 . . . 336 | 332/336 (98%) | |
| ABG96271 | Human immunoglobulin superfamily | 1 . . . 336 | 332/336 (98%) | 0.0 |
| | protein IGSFP-9 - Homo sapiens, 336 | 1 . . . 336 | 332/336 (98%) | |
| | aa. [WO200272794-A2, 19-SEP-2002] | | | |
| AAU84386 | Novel human secreted or membrane- | 1 . . . 336 | 325/336 (96%) | 0.0 |
| | associated protein #25 - Homo sapiens, | 1 . . . 336 | 329/336 (97%) | |
| | 336 aa. [WO200204600-A2, 17-JAN-2002] | | | |
| ABG94639 | Human NOV5d protein - Homo sapiens, | 1 . . . 336 | 322/336 (95%) | 0.0 |
| | 336 aa. [WO200266643-A2, 29-AUG-2002] | 1 . . . 336 | 326/336 (96%) | |
| ABG94637 | Human NOV5b protein - Homo sapiens, | 18 . . . 336 | 315/319 (98%) | 0.0 |
| | 319 aa. [WO200266643-A2, 29-AUG-2002] | 1 . . . 319 | 315/319 (98%) | |

In a BLAST search of public sequence databases, the NOV11a protein was found to have homology to the proteins shown in the BLASTP data in Table 11E.

TABLE 11E
Public BLASTP Results for NOV11a

| | | NOV11a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8HW98 | Adult male hypothalamus cDNA, RIKEN | 12 . . . 336 | 313/325 (96%) | 0.0 |
| | full-length enriched library, | 1 . . . 325 | 318/325 (97%) | |
| | clone: A230106M20 product: weakly similar | | | |
| | to limbic system-associated membrane | | | |
| | protein precursor (E19S) (CHLAMP, G19- | | | |
| | isoform) - Mus musculus (Mouse), 325 aa | | | |
| | (fragment). | | | |
| P11834 | Opioid binding protein/cell adhesion | 11 . . . 329 | 168/328 (51%) | 1e−84 |
| | molecule precursor (OBCAM) (Opioid- | 12 . . . 337 | 224/328 (68%) | |
| | binding cell adhesion molecule) (OPCML) - | | | |
| | Bos taurus (Bovine), 345 aa. | | | |
| Q14982 | Opioid binding protein/cell adhesion | 11 . . . 329 | 166/328 (50%) | 3e−84 |
| | molecule precursor (OBCAM) (Opioid- | 12 . . . 337 | 224/328 (67%) | |
| | binding cell adhesion molecule) (OPCML) - | | | |
| | Homo sapiens (Human), 345 aa. | | | |
| JC1238 | opioid-binding protein (clone DUZ1) - rat, | 12 . . . 333 | 163/326 (50%) | 5e−83 |
| | 338 aa. | 8 . . . 325 | 221/326 (67%) | |
| P32736 | Opioid binding protein/cell adhesion | 11 . . . 333 | 164/329 (49%) | 5e−83 |
| | molecule precursor (OBCAM) (Opioid- | 12 . . . 332 | 224/329 (67%) | |
| | binding cell adhesion molecule) (OPCML) - | | | |
| | Rattus norvegicus (Rat), 345 aa. | | | |

PFam analysis predicts that the NOV11a protein contains the domains shown in the Table 11F.

TABLE 11F
Domain Analysis of NOV11a

| | | Identities/ | |
|---|---|---|---|
| | | Similarities | |
| Pfam | NOV11a | for the | Expect |
| Domain | Match Region | Matched Region | Value |
| ig | 47 . . . 114 | 16/72 (22%) | 0.00065 |
| | | 40/72 (56%) | |
| ig | 147 . . . 197 | 16/54 (30%) | 0.00057 |
| | | 35/54 (65%) | |
| ig | 231 . . . 293 | 12/66 (18%) | 2.7e-05 |
| | | 45/66 (68%) | |

### Example 12.

The NOV12 clone was analyzed, and the nucleotide and encoded polypeptide sequences are shown in Table 12A.

TABLE 12A
NOV12 Sequence Analysis

| | | |
|---|---|---|
| NOV12a, CG51373-11 | SEQ ID NO: 319 | 3212 bp |
| DNA Sequence | ORF Start: ATG at 1 | ORF Stop: TAG at 2272 |

ATGCTGAGCCTCCTCGTCTGGATCCTCACTCTCTCCGATACTTTCTCCCAAGGGACCCAGACCCGCTT
CAGCCAGGAGCCAGCTGACCAGACGGTGGTGGCTGGACAGCGGGCCGTGCTCCCCTGTGTGCTGCTCA
ACTACTCTGGAATTGTGCAATGGACCAAGGACGGGCTGGCCCTGGGCATGGGCCAGGGCCTCAAAGCC
TGGCCACGGTACCGGGTTGTGGGCTCCGCAGACGCTGGGCAGTACAACCTGGAGATCACAGATGCTGA
GCTCTCTGACGACGCCTCTTACGAGTGCCAGGCCACGGAGGCCGCCCTGCGCTCTCGGCGGGCCAAAC
TCACCGTGCTCATCCCCCCAGAGGACACCAGGATTGACGGAGGCCCTGTGATTCTACTGCAGGCAGGC
ACCCCCCACAACCTCACATGCCGGGCCTTCAATGCGAAGCCTGCTGCCACCATCATCTGGTTCCGGGA
CGGGACGCAGCAGGAGGGCGCTGTGGCCAGCACGGAATTGCTGAAGGATGGGAAGAGGGAGACCACCG
TGAGCCAACTGCTTATTAACCCCACGGACCTGGACATAGGGCGTGTCTTCACTTGCCGAAGCATGAAC
GAAGCCATCCCTAGTGGCAAGGAGACTTCCATCGAGCTGGATGTGCACCACCCTCCTACAGTGACCCT
GTCCATTGAGCCACAGACGGTGCAGGAGGGTGAGCGTGTTGTCTTTACCTGCCAGGCCACAGCCAACC
CCGAGATCTTGGGCTACAGGTGGGCCAAAGGGGGTTTCTTGATTGAAGACGCCCACGAGAGTCGCTAT
GAGACAAATGTGGATTATTCCTTTTTCACGGAGCCTGTGTCTTGTGAGGTTCACAACAAAGTGGGAAG
CACCAATGTCAGCACTTTAGTAAATGTCCACTTTGCTCCCCGGATTGTAGTTGACCCCAAACCCACAA
CCACAGACATTGGCTCTGATGTGACCCTTACCTGTGTCTGGGTTGGGAATCCCCCCCTCACTCTCACC
TGGACCAAAAAGGACTCAAATATGGTCCTGAGTAACAGCAACCAGCTGCTGCTGAAGTCGGTGACTCA
GGCAGACGCTGGCACCTACACCTGCCGGGCCATCGTGCCTCGAATCGGAGTGGCTGAGCGGGAGGTGC
CGCTCTATGTGAACGGGCCCCCCATCATCTCCAGTGAGGCAGTGCAGTATGCTGTGAGGGGTGACGGT
GGCAAGGTGGAGTGTTTCATTGGGAGCACACCACCCCCAGACCGCATAGCATGGGCCTGGAAGGAGAA
CTTCTTGGAGGTGGGGACCCTGGAACGCTATACAGTGGAGAGGACCAACTCAGGCAGTGGGGTGCTAT
CCACGCTCACCATCAACAATGTCATGGAGGCCGACTTTCAGACTCACTACAACTGCACCGCCTGGAAC
AGCTTCGGGCCAGGCACAGCCATCATCCAGCTGGAAGAGCGAGAGGTGTTACCTGTGGGCATCATAGC
TGGGGCCACCATCGGCGCGAGCATCCTGCTCATCTTCTTCTTCATCGCCTTGGTATTCTTCCTCTACC
GGCGCCGCAAAGGCAGTCGCAAAGACGTGACCCTGAGGAAGCTGGATATCAAGGTGGAGACAGTGAAC
CGAGAGCCACTTACGATGCATTCTGACCGGGAGGATGACACCGCCAGCGTCTCCACAGCAACCCGGGT
CATGAAGGCCATCTACTCGTCGTTTAAGGATGATGTGGATCTGAAGCAGGACCTGCGCTGCGACACCA
TCGACACCCGGGAGGAGTATGAGATGAAGGACCCCACCAATGGCTACTACAACGTGCGTGCCCATGAA
GACCGCCCGTCTTCCAGGGCAGTGCTCTATGCTGACTACCGTGCCCCTGGCCCTGCCCGCTTCGACGG
CCGCCCCTCATCCCGTCTCTCCCACTCCAGCGGCTATGCCCAGCTCAACACCTATAGCCGGGGCCCTG
CCTCTGACTATGGCCCTGAGCCCACACCCCCTGGCCCTGCTGCCCCAGCTGGCACTGACACAACCAGC
CAGCTGTCCTACGAGAACTATGAGAAGTTCAACTCCCATCCCTTCCCTGGGGCAGCTGGGTACCCCAC
CTACCGACTGGGCTACCCCCAGGCCCCACCCTCTGGCCTGGAGCGGACCCCATATGAGGCGTATGACC
CCATTGGCAAGTACGCCACAGCCACTCGATTCTCCTACACCTCCCAGCACTCGGACTACGGCCAGCGA
TTCCAGCAGCGCATGCAGACTCACGTGTAGGGGCCAGAGCCTGGCTGGGGCATCTCTGCGGGGCAGAG
GAGAAGGCTTTCGCAGCTGTTCCCTGATATTCAGGGACATTGCTCATTGCTCCCTTCTCGGACCAGCC
TTCTTCCTCCCACCATGGCAGGTGGGGAGCAGGTCTCCCAGAGACACCCCGTCCCGAGGATGGTGCTC
TGTGCATGCCCCAGCCTCCTGGGCCTGCCCTTCCCTCTTCTTCGGGAGGATGTGTCTCTTCTGACCTG
CACTCTTGCCTGACCCTAGAATGGGGACAGGGAAAGTGAAGGTTAGGGAAAGCAGAGGGGGGCACTTT
TTAGCATTCCCTTTCTATCCCACCCCTCTGATCTCCCATAAGTGGAAATGGGGGTACCCAGGGATGGG
CAGGCTTTGGCCTAGGGACATGAAGTATGGGAGTGGGTGGCTGTGGCACAGACAGGTGGAAAACGGGA
TAGCCTGGCCAGTCCCTCTGTTGTCTGCATTCGTGCCCTGGGTGCCTCTCTCCTTCCTCAGGGTACTG
CAGAAGGGAGCGAACAGGGTACTGTTCGCTCTTGTCTACAGAACAGCCCTGGCACTGCATTCAAATCC
AGTCTTCATTCAGCTGGGATCAAAATGCCAGTCACCTTGGCTACCCACTGTGGACAGCTGTCTGTCAG
CATGCAGAGGGATCCAGGAATCCCCCCGGCAGCACGGCCCGCTTTCCTTCTCCTCCATGCTGGGCCAG
CCAGATAAGTCAGGGTCCTGGTGGAGAAAGAAAGGCTAGGACCATGTCCTCATTGACCCAGATACTGC
TGTGTGCTGCACAGCAGTGAACCAACACTAGAGGGAGCCACACAAGCCTCCTCTCCCCAGTCTGCCCC
ACTTCCTGGCTTTAACTCTTGAGCTGGTTTGGGGAGTGGTGAGGTAGGGGTGGGGGTGCTGTAGGCTC
TTTTTCAAAAAAAAAC

| | | | |
|---|---|---|---|
| NOV12a, CG51373-11 | SEQ ID NO: 320 | 757 aa | MW at 83534.8kD |
| Protein Sequence | | | |

MLSLLVWILTLSDTFSQGTQTRFSQEPADQTVVAGQRAVLPCVLLNYSGIVQWTKDGLALGMGQGLKA
WPRYRVVGSADAGQYNLEITDAELSDDASYECQATEAALRSRRAKLTVLIPPEDTRIDGGPVILLQAG
TPHNLTCRAFNAKPAATIIWFRDGTQQEGAVASTELLKDGKRETTVSQLLINPTDLDIGRVFTCRSMN
EAIPSGKETSIELDVHHPPTVTLSIEPQTVQEGERVVFTCQATANPEILGYRWAKGGFLIEDAHESRY
ETNVDYSFFTEPVSCEVHNKVGSTNVSTLVNVHFAPRIVVDPKPTTTDIGSDVTLTCVWVGNPPLTLT
WTKKDSNMVLSNSNQLLLKSVTQADAGTYTCRAIVPRIGVAEREVPLYVNGPPIISSEAVQYAVRGDG
GKVECFIGSTPPPDRIAWAWKENFLEVGTLERYTVERTNSGSGVLSTLTINNVMEADFQTHYNCTAWN
SFGPGTAIIQLEEREVLPVGIIAGATIGASILLIFFFIALVFFLYRRRKGSRKDVTLRKLDIKVETVN
REPLTMHSDREDDTASVSTATRVMKAIYSSFKDDVDLKQDLRCDTIDTREEYEMKDPTNGYYNVRAHE
DRPSSRAVLYADYRAPGPARFDGRPSSRLSHSSGYAQLNTYSRGPASDYGPEPTPPGPAAPAGTDTTS
QLSYENYEKFNSHPFPGAAGYPTYRLGYPQAPPSGLERTPYEAYDPIGKYATATRFSYTSQHSDYGQR
FQQRIYIQTHV

| | | |
|---|---|---|
| NOV12b, CG51373-10 | SEQ ID NO: 321 | 3430 bp |
| DNA Sequence | ORF Start: ATG at 351 | ORF Stop: TAG at 2490 |

CTCTCCGATACTTTCTCCCAAGGGTCAGCTGCTTCTTCATTCCAAGTGGACAAGGAGCCAGCTGCTCA
CTGTCCTTGAGAGACTTCAGCGAGAGACCAGGGTGTCCAGGCTCCATGCAGGAAAGCCATGCGTATAA
ATTCCACCTCTGAGCCAGGCCTCACCAGCAAGCCCACTCTTAAGCCCTTGACTTGGGCTCCAGGGGCC
ATGGGAAGGAGAAACGGACCCAGACCCGCTTCAGCCAGGAGCCAGCTGACCAGACGGTGGTGGCTGGA
CAGCGGGCCGTGCTCCCCTGTGTGCTGCTCAACTACTCTGGAATTGTGCAATGGACCAAGGACGGGCT
GGCCCTGGGCATGGGCCAGGCCCTCAAAGCCTGGCCACGGTACCGGGTTGTGGGCTCCGCAGACGCTG
GGCAGTACAACCTGGAGATCACAGATGCTGAGCTCTCTGACGACGCCTCTTACGAGTGCCAGGCCACG
GAGGCCGCCCTGCGCTCTCGGCGGGCCAAACTCACCGTGCTCATCCCCCCAGAGGACACCAGGATTGA
CGGAGGCCCTGTGATTCTACTGCAGGCAGGCACCCCCCACAACCTCACATGCCGGGCCTTCAATGCGA
AGCCTGCTGCCACCATCATCTGGTTCCGGGACGGGACGCAGCAGGAGGGCGCTGTGGCCAGCACGGAA
TTGCTGAAGGATGGGAAGAGGGAGACCACCGTGAGCCAACTGCTTATTAACCCCACGGACCTGGACAT
AGGGCGTGTCTTCACTTGCCGAAGCATGAACGAAGCCATCCCTAGTGGCAAGGAGACTTCCATCGAGC
TGGATGTGCACCACCCTCCTACAGTGACCCTGTCCATTGAGCCACAGACGGTGCAGGAGGGTGAGCGT
GTTGTCTTTACCTGCCAGGCCACAGCCAACCCCGAGATCTTGGGCTACAGGTGGGCCAAAGGGGGTTT
CTTGATTGAAGACGCCCACGAGAGTCGCTATGAGACAAATGTGGATTATTCCTTTTTCACGGAGCCTG
TGTCTTGTGAGGTTCACAACAAAGTGGGAAGCACCAATGTCAGCACTTTAGTAAATGTCCACTTTGCT
CCCCGGATTGTAGTTGACCCCAAACCCACAACCACAGACATTGGCTCTGATGTGACCCTTACCTGTGT
CTGGGTTGGGGAAATCCCCCCCTCACTCTCACCTGGACCAAAAAGGACTCAAATATTGGGGCCCTGGC
TTCTTGGTTCCCCACCCGAGGCTGCTCTCTCTGCCCAGGTCCTGAGTAACAGCAACCAGCTGCTGCTG
AAGTCGGTGACTCAGGCAGACGCTGGCACCTACACCTGCCGGGCCATCGTGCCTCGAATCGGAGTGGC
TGAGCGGGAGGTGCCGCTCTATGTGAACGGGCCCCCCATCATCTCCAGTGAGGCAGTGCAGTATGCTG
TGAGGGGTGACGGTGGCAAGGTGGAGTGTTTCATTGGGAGCACACCACCCCCAGACCGCATAGCATGG
GCCTGGAAGGAGAACTTCTTGGAGGTGGGGACCCTGGAACGCTATACAGTGGAGAGGACCAACTCAGG
CAGTGGGGTGCTATCCACGCTCACCATCAACAATGTCATGGAGGCCGACTTTCAGACTCACTACAACT
GCACCGCCTGGAACAGCTTCGGGCCAGGCACAGCCATCATCCAGCTGGAAGAGCGAGAGGTGTTACCT
GTGGGCATCATAGCTGGGGCCACCATCGGCGCGAGCATCCTGCTCATCTTCTTCTTCATCGCCTTGGT
ATTCTTCCTCTACCGGCGCCGCAAAGGCAGTCGCAAAGACGTGACCCTGAGGAAGCTGGATATCAAGG
TGGAGACAGTGAACCGAGAGCCACTTACGATGCATTCTGACCGGGAGGATGACACCGCCAGCGTCTCC
ACAGCAACCCGGGTCATGAAGGCCATCTACTCGTCGTTTAAGGATGATGTGGATCTGAAGCAGGACCT
GCGCTGCGACACCATCGACACCCGGGAGGAGTATGAGATGAAGGACCCCACCAATGGCTACTACAACG
TGCGTGCCCATGAAGACCGCCCGTCTTCCAGGGCAGTGCTCTATGCTGACTACCGTGCCCCTGGCCCT
GCCCGCTTCGACGGCCGCCCCTCATCCCGTCTCTCCCACTCCAGCGGCTATGCCCAGCTCAACACCTA
TAGCCGGGGCCCTGCCTCTGACTATGGCCCTGAGCCCACACCCCCTGGCCCTGCTGCCCCAGCTGGCA
CTGACACAACCAGCCAGCTGTCCTACGAGAACTATGAGAAGTTCAACTCCCATCCCTTCCCTGGGGCA
GCTGGGTACCCCACCTACCGACTGGGCTACCCCCAGGCCCCACCCTCTGGCCTGGAGCGGACCCCATA
TGAGGCGTATGACCCCATTGGCAAGTACGCCACAGCCACTCGATTCTCCTACACCTCCCAGCACTCGG
ACTACGGCCAGCGATTCCAGCAGCGCATGCAGACTCACGTGTAGGGGCCAGAGCCTGGCTGGGGCATC
TCTGCGGGGCAGAGGAGAAGGCTTTCGCAGCTGTTCCCTGATATTCAGGGACATTGCTCATTGCTCCC
TTCTCGGACCAGCCTTCTTCCTCCCACCATGGCAGGTGGGGAGCAGGTCTCCCAGAGACACCCCGTCC
CGAGGATGGTGCTCTGTGCATGCCCCAGCCTCCTGGGCCTGCCCTTCCCTCTTCTTCGGGAGGATGTG
TCTCTTCTGACCTGCACTCTTGCCTGACCCTAGAATGGGGACAGGGAAAGTGAAGGTTAGGGAAAGCA
GAGGGGGGCACTTTTTAGCATTCCCTTTCTATCCCACCCCTCTGATCTCCCATAAGTGGAAATGGGGC
TACCCAGGGATGGGCAGGCTTTGGCCTAGGGACATGAAGTATGGGAGTGGGTGGCTGTGGCACAGACA
GGTGGAAAACGGGATAGCCTGGCCAGTCCCTCTGTTGTCTGCATTCGTGCCCTGGGTGCCTCTCTCCT
TCCTCAGGGTACTGCAGAAGGGAGCGAACAGGGTACTGTTCGCTCTTGTCTACAGAACAGCCCTGGCA
CTGCATTCAAATCCAGTCTTCATTCAGCTGGGATCAAAATGCCAGTCACCTTGGCTACCCACTGTGGA
CAGCTGTCTGTCAGCATGCAGAGGGATCCAGGAATCCCCCCGGCAGCACGGCCCGCTTTCCTTCTCCT
CCATGCTGGGCCAGCCAGATAAGTCAGGGTCCTGGTGGAGAAAGAAAGGCTAGGACCATGTCCTCATT
GACCCAGATACTGCTGTGTGCTGCACAGCAGTGAACCAACACTAGAGGGAGCCACACAAGCCTCCTCT
CCCCAGTCTGCCCCACTTCCTGGCTTTAACTCTTGAGCTGGTTTGGGGAGTGGTGAGGTAGGGGTGGG
GGTGCTGTAGGCTCTTTTTCAAAAAAAAAC

| | | | |
|---|---|---|---|
| NOV12b, CG51373-10 | SEQ ID NO: 322 | 713 aa | MW at 78491.0kD |
| Protein Sequence | | | |

MGQALKAWPRYRVVGSADAGQYNLEITDAELSDDASYECQATEAALRSRRAKLTVLIPPEDTRIDGGP
VILLQAGTPHNLTCRAFNAKPAATIIWFRDGTQQEGAVASTELLKDGKRETTVSQLLINPTDLDIGRV
FTCRSMNEAIPSGKETSIELDVHHPPTVTLSIEPQTVQEGERVVFTCQATANPETLGYRWAKGGFLIE
DAHESRYETNVDYSFFTEPVSCEVHNKVGSTNVSTLVNVHFAPRIVVDPKPTTTDIGSDVTLTCVWVG
EIPPSLSPGPKRTQILGPWLLGSPPEAALSAQVLSNSNQLLLKSVTQADAGTYTCRAIVPRIGVAERE
VPLYVNGPPIISSEAVQYAVRGDGGKVECFIGSTPPPDRIAWAWKENFLEVGTLERYTVERTNSGSGV
LSTLTINNVMEADFQTHYNCTAWNSFGPGTAIIQLEEREVLPVGIIAGATIGASTLLIFFFIALVFFL
YRRRKGSRKDVTLRKLDIKVETVNREPLTMHSDREDDTASVSTATRVMKAIYSSFKDDVDLKQDLRCD
TIDTREEYEMKDPTNGYYNVRAHEDRPSSRAVLYADYRAPGPARFDGRPSSRLSHSSGYAQLNTYSRG
PASDYGPEPTPPGPAAPAGTDTTSQLSYENYEKFNSHPFPGAAGYPTYRLGYPQAPPSGLERTPYEAY
DPIGKYATATRFSYTSQHSDYGQRFQQRMQTHV

| | | |
|---|---|---|
| NOV12c, CG51373-08 | SEQ ID NO: 323 | 2128 bp |
| DNA Sequence | ORF Start: ATG at 31 | ORF Stop: TAG at 2119 |

CAATGGACCAAGGACGGGCTGGCCCTGGGCATGGGCCAGGGCCTCAAAGCCTGGCCACGGTACCGGGT
TGTGGGCTCCGCAGACGCTGGGCAGTACAACCTGGAGATCACAGATGCTGAGCTCTCTGACGACGCCT
CTTACGAGTGCCAGGCCACGGAGGCCGCCCTGCGCTCTCGGCGGGCCAAACTCACCGTGCTCATCCCC
CCAGAGGACACCAGGATTGACGGAGGCCCTGTGATTCTACTGCAGGCAGGCACCCCCCACAACCTCAC
ATGCCGGGCCTTCAATGCGAAGCCTGCTGCCACCATCATCTGGTTCCGGGACGGGACGCAGCAGGAGG
GCGCTGTGGCCAGCACGGAATTGCTGAAGGATGGGAAGAGGGAGACCACCGTGAGCCAACTGCTTATT
AACCCCACGGACCTGGACATAGGGCGTGTCTTCACTTGCCGAAGCATGAACGAAGCCATCCCTAGTGG
CAAGGAGACTTCCATCGAGCTGGATGTGCACCACCCTCCTACAGTGACCCTGTCCATTGAGCCACAGA
CGGTGCAGGAGGGTGAGCGTGTTGTCTTTACCTGCCAGGCCACAGCCAACCCCGAGATCTTGGACTAC
AGGTGGGCCAAAGGGGGTTTCTTGATTGAAGACGCCCACGAGAGTCGCTATGAGACAAATGTGGATTA
TTCCTTTTTCACGGAGCCTGTGTCTTGTGAGGTTCACAACAAAGTGGGAAGCACCAATGTCAGCACTT
TAGTAAATGTCCACTTTGCTCCCCGGATTGTAGTTGACCCCAAACCCACAACCACAGACATTGGCTCT
GATGTGACCCTTACCTGTGTCTGGGTTGGGAATCCCCCCCTCACTCTCACCTGGACCAAAAAGGACTC
AAATATGGTCCTGAGTAACAGCAACCAGCTGCTGCTGAAGTCGGTGACTCAGGCAGACGCTGGCACCT
ACACCTGCCGGGCCATCGTGCCTCGAATCGGAGTGGCTGAGCGGGAGGTGCCGCTCTATGTGAACGGG
CCCCCCATCATCTCCAGTGAGGCAGTGCAGTATGCTGTGAGGGGTGACGGTGGCAAGGTGGAGTGTTT
CATTGGGAGCACACCACCCCCAGACCGCATAGCATGGGCCTGGAAGGAGAACTTCTTGGAGGTGGGGA
CCCTGGAACGCTATACAGTGGAGAGGACCAACTCAGGCAGTGGGGTGCTATCCACGCTCACCATCAAC
AATGTCATGGAGGCCGACTTTCAGACTCACTACAACTGCACCGCCTGGAACAGCTTCGGGCCAGGCAC
AGCCATCATCCAGCTGGAAGAGCGAGAGGTGTTACCTGTGGGCATCATAGCTGGGGCCACCATCGGCG
CGAGCATCCTGCTCATCTTCTTCTTCATCGCCTTGGTATTCTTCCTCTACCGGCGCCGCAAAGGCAGT
CGCAAAGACGTGACCCTGAGGAAGCTGGATATCAAGGTGGAGACAGTGAACCGAGAGCCACTTACGAT
GCATTCTGACCGGGAGGATGACACCGCCAGCGTCTCCACAGCAACCCGGGTCATGAAGGCCATCTACT
CGTCGTTTATGGATGATGTGGATCTGAAGCAGGACCTGCGCTGCGACACCATCGACACCCGGGAGGAG
TATGAGATGAAGGACCCCACCAATGGCTACTACAACGTGCGTGCCCATGAAGACCGCCCGTCTTCCAG
GGCAGTGCTCTATGCTGACTACCGTGCCCCTGGCCCTGCCCGCTTCGACGGCCGCCCCTCATCCCGTC
TCTCCCACTCCAGCGGCTATGCCCAGCTCAACACCTATAGCCGGGGCCCTGCCTCTGACTATGGCCCT
GAGCCCACACCCCCTGGCCCTGCTGCCCCAGCTGGCACTGACACAACCAGCCAGCTGTCCTACGAGAA
CTATGAGAAGTTCAACTCCCATCCCTTCCCTGGGGCAGCTGGGTACCCCACCTACCGACTGGGCTACC
CCCAGGCCCCACCCTCTGGCCTGGAGCGGACCCCATATGAGGCGTATGACCCCATTGGCAAGTACGCC
ACAGCCACTCGATTCTCCTACACCTCCCAGCACTCGGACTACGGCCAGCGATTCCAGCAGCGCATGCA
GACTCACGTGTAGGGGCCAG

| | | | |
|---|---|---|---|
| NOV12c, CG51373-08 | SEQ ID NO: 324 | 696 aa | MW at 76975.3kD |
| Protein Sequence | | | |

MGQGLKAWPRYRVVGSADAGQYNLEITDAELSDDASYECQATEAALRSRRAKLTVLIPPEDTRIDGGP
VILLQAGTPHNLTCRAFNAKPAATIIWFRDGTQQEGAVASTELLKDGKRETTVSQLLINPTDLDIGRV
FTCRSMNEAIPSGKETSIELDVHHPPTVTLSIEPQTVQEGERVVFTCQATANPEILDYRWAKGGFLIE
DAHESRYETNVDYSFFTEPVSCEVHNKVGSTNVSTLVNVHFAPRIVVDPKPTTTDIGSDVTLTCVWVG
NPPLTLTWTKKDSNNVLSNSNQLLLKSVTQADAGTYTCHAIVPRIGVAEREVPLYVNGPPIISSEAVQ
YAVRGDGGKVECFIGSTPPPDRIAWAWKENFLEVGTLERYTVERTNSGSGVLSTLTINNVMEADFQTH
YNCTAWNSFGPGTAIIQLEEREVLPVGIIAGATIGASILLIFFFIALVFFLYRRRKGSRKDVTLRKLD
IKVETVNREPLTMHSDREDDTASVSTATRVMKAIYSSFMDDVDLKQDLRCDTIDTREEYEMKDPTNGY
YNVRAHEDRPSSRAVLYADYRAPGPARFDGRPSSRLSHSSGYAQLNTYSRGPASDYGPEPTPPGPAAP
AGTDTTSQLSYENYEKFNSHPFPGAAGYPTYRLGYPQAPPSGLERTPYEAYDPIGKYATATRFSYTSQ
HSDYGQRFQQRMQTHV

| | | |
|---|---|---|
| NOV12d, 237582097 | SEQ ID NO: 325 | 2100 bp |
| DNA Sequence | ORF Start: at 1 | ORF Stop: end of sequence |

GGATCCATGGGCCAGGGCCTCAAAGCCTGGCCACGGTACCGGGTTGTGGGCTCCGCAGACGCTGGGCA
GTACAACCTGGAGATCACAGATGCTGAGCTCTCTGACGACGCCTCTTACGAGTGCCAGGCCACGGAGG
CCGCCCTGCGCTCTCGGCGGGCCAAACTCACCGTGCTCATCCCCCCAGAGGACACCAGGATTGACGGA
GGCCCTGTGATTCTACTGCAGGCAGGCACCCCCCACAACCTCACATGCCGGGCCTTCAATGCGAAGCC
TGCTGCCACCATCATCTGGTTCCGGGACGGGACGCAGCAGGAGGGCGCTGTGGCCAGCACGGAATTGC
TGAAGGATGGGAAGAGGGAGAACACCGTGAGCCAACTGCTTATTAACCCCACGGACCTGGACATAGGG
CGTGTCTTCACTTGCCGAAGCATGAACGAAGCCATCCCTAGTGGCAAGGAGACTTCCATCGAGCTGGA
TGTGCACCACCCTCCTACAGTGACCCTGTCCATTGAGCCACAGACGGTGCAGGAGGGTGAGCGTGTTG
TCTTTACCTGCCAGGCCACAGCCAACCCCGAGATCTTGGACTACAGGTGGGCCAAAGGGGGTTTCTTG
ATTGAAGACGCCCACGAGAGTCGCTATGAGACAAATGTGGATTATTCCTTTTTCACGGAGCCTGTGTC
TTGTGAGGTTCACAACAAAGTGGGAAGCACCAATGTCAGCACTTTAGTAAATGTCCACTTTGCTCCCC
GGATTGTAGTTGACCCCAAACCCACAACCACAGACATTGGCTCTGATGTGACCCTTACCTGTGTCTGG
GTTGGGAATCCCCCCCTCACTCTCACCTGGACCAAAAAGGACTCAAATATGGTCCTGAGTAACAGCAA
CCAGCTGCTGCTGAAGTCGGTGACTCAGGCAGACGCTGGCACCTACACCTGCCGGGCCATCGTGCCTC
GAATCGGAGTGGCTGAGCGGGAGGTGCCGCTCTATGTGAACGGGCCCCCCATCATCTCCAGTGAGGCA
GTGCAGTATGCTGTGAGGGGTGACGGTGGCAAGGTGGAGTGTTTCATTGGGAGCACACCACCCCCAGA
CCGCATAGCATGGGCCTGGAAGGAGAACTTCTTGGAGGTGGGGACCCTGGAACGCTATACAGTGGAGA
GGACCAACTCAGGCAGTGGGGTGCTATCCACGCTCACCATCAACAATGTCATGGAGGCCGACTTTCAG
ACTCACTACAACTGCACCGCCTGGAACAGCTTCGGGCCAGGCACAGCCATCATCCAGCTGGAAGAGCG
AGAGGTGTTACCTGTGGGCATCATAGCTGGGGCCACCATCGGCGCGAGCATCCTGCTCATCTTCTTCT
TCATCGCCTTGGTATTCTTCCTCTACCGGCGCCGCAAAGGCAGTCGCAAAGACGTGACCCTGAGGAAG
CTGGATATCAAGGTGGAGACAGTGAACCGAGAGCCACTTACGATGCATTCTGACCGGGAGGATGACAC
CGCCAGCGTCTCCACAGCAACCCGGGTCATGAAGGCCATCTACTCGTCGTTTATGGATGATGTGGATC
TGAAGCAGGACCTGCGCTGCGACACCATCGACACCCGGGAGGAGTATGAGATGAAGGACCCCACCAAT
GGCTACTACAACGTGCGTGCCCATGAAGACCGCCCGTCTTCCAGGGCAGTGCTCTATGCTGACTACCG
TGCCCCTGGCCCTGCCCGCTTCGACGGCCGCCCCTCATCCCGTCTCTCCCACTCCAGCGGCTATGCCC
AGCTCAACACCTATAGCCGGGGCCCTGCCTCTGACTATGGCCCTGAGCCCACACCCCCTGGCCCTGCT
GCCCCAGCTGGCACTGACACAACCAGCCAGCTGTCCTACGAGAACTATGAGAAGTTCAACTCCCATCC
CTTCCCTGGGGCAGCTGGGTACCCCACCTACCGACTGGGCTACCCCCAGGCCCCACCCTCTGGCCTGG
AGCGGACCCCATATGAGGCGTATGACCCCATTGGCAAGTACGCCACAGCCACTCGATTCTCCTACACC
TCCCAGCACTCGGACTACGGCCAGCGATTCCAGCAGCGCATGCAGACTCACGTGCTCGAG

| | | | |
|---|---|---|---|
| NOV12d, 237582097 | SEQ ID NO: 326 | 700 aa | MW at 77361.7kD |
| Protein Sequence | | | |

GSMGQGLKAWPRYRVVGSADAGQYNLEITDAELSDDASYECQATEAALRSRPAKLTVLIPPEDTRIDG
GPVILLQAGTPHNLTCRAFNAKPAATIIWFRDGTQQEGAVASTELLKDGKRETTVSQLLINPTDLDIG
RVFTCRSMNEAIPSGKETSIELDVHHPPTVTLSIEPQTVQEGERVVFTCQATANPEILDYRWAKGGFL
IEDAHESRYETNVDYSFFTEPVSCEVHNKVGSTNVSTLVNVHFAPRIVVDPKPTTTDIGSDVTLTCVW
VGNPPLTLTWTKKDSNMVLSNSNQLLLKSVTQADAGTYTCRAIVPRIGVAEREVPLYVNGPPIISSEA
VQYAVRGDGGKVECFIGSTPPPDRIAWAWKENFLEVGTLERYTVERTNSGSGVLSTLTINNVMEADFQ
THYNCTAWNSFGPGTAIIQLEEREVLPVGIIAGATIGASILLIFFFIALVFFLYRRRKGSRKDVTLRK
LDIKVETVNREPLTMHSDREDDTASVSTATRVMKAIYSSFMDDVDLKQDLRCDTIDTREEYEMKDPTN
GYYNVRAHEDRPSSRAVLYADYRAPGPARFDGRPSSRLSHSSGYAQLNTYSRGPASDYGPEPTPPGPA
APAGTDTTSQLSYENYEKFNSHPFPGAAGYPTYRLGYPQAPPSGLERTPYEAYDPIGKYATATRFSYT
SQHSDYGQRFQQRMQTHVLE

| | | |
|---|---|---|
| NOV12e, 294385961 | SEQ ID NO: 327 | 2290 bp |
| DNA Sequence | ORF Start: at 2 | ORF Stop: end of sequence |

CACCAAGCTTATGCTGAGCCTCCTCGTCTGGATCCTCACTCTCTCCGATACTTTCTCCCAAGGGACCC
AGACCCGCTTCAGCCAGGAGCCAGCTGACCAGACGGTGGTGGCTGGACAGCGGGCCGTGCTCCCCTGT
GTGCTGCTCAACTACTCTGGAATTGTGCAATGGACCAAGGACGGGCTGGCCCTGGGCATGGGCCAGGG
CCTCAAAGCCTGGCCACGGTACCGGGTTGTGGGCTCCGCAGACGCTGGGCAGTACAACCTGGAGATCA
CAGATGCTGAGCTCTCTGACGACGCCTCTTACGAGTGCCAGGCCACGGAGGCCGCCCTGCGCTCTCGG
CGGGCCAAACTCACCGTGCTCATCCCCCCAGAGGACACCAGGATTGACGGAGGCCCTGTGATTCTACT
GCAGGCAGGCACCCCCCACAACCTCACATGCCGGGCCTTCAATGCGAAGCCTGCTGCCACCATCATCT
GGTTCCGGGACGGGACGCAGCAGGAGGGCGCTGTGGCCAGCACGGAATTGCTGAAGGATGGGAAGAGG
GAGACCACCGTGAGCCAACTGCTTATTAACCCCACGGACCTGGACATAGGGCGTGTCTTCACTTGCCG
AAGCATGAACGAAGCCATCCCTAGTGGCAAGGAGACTTCCATCGAGCTGGATGTGCACCACCCTCCTA
CAGTGACCCTGTCCATTGAGCCACAGACGGTGCAGGAGGGTGAGCGTGTTGTCTTTACCTGCCAGGCC
ACAGCCAACCCGGAGATCTTGGGCTACAGGTGGGCCAAAGGGGGTTTCTTGATTGAAGACGCCCACGA
GAGTCGCTATGAGACAAATGTGGATTATTCCTTTTTCACGGAGCCTGTGTCTTGTGAGGTCCACAACA
AAGTGGGAAGCACCAATGTCAGCACTTTAGTAAATGTCCACTTTGCTCCCCGGATTGTAGTTGACCCC
AAACCCACAACCACAGACATTGGCTCTGATGTGACCCTTACCTGTGTCTGGGTTGGGAATCCCCCCCT
CACTCTCACCTGGACCAAAAAGGACTCAAATATGGTCCTGAGTAACAGCAACCAGCTGCTGCTGAAGT
CGGTGACTCAGGCAGACGCTGGCACCTACACCTGCCGGGCCATCGTGCCTCGAATCGGAGTGGCTGAG
CGGGAGGTGCCGCTCTATGTGAACGGGCCCCCCATCATCTCCAGTGAGGCAGTGCAGTATGCTGTGAG
GGGTGACGGTGGCAAGGTGGAGTGTTTCATTGGGAGCACACCACCCCCAGACCGCATAGCATGGGCCT
GGAAGGAGAACTTCTTGGAGGTGGGGACCCTGGAACGCTATACAGTGGAGAGGACCAACTCAGGCAGT
GGGGTGCTATCCACGCTCACCATCAACAATGTCATGGAGGCCGACTTTCAGACTCACTACAACTGCAC
CGCCTGGAACAGCTTCGGGCCAGGCACAGCCATCATCCAGCTGGAAGAGCGAGAGGTGTTACCTGTGG
GCATCATAGCTGGGGCCACCATCGGCGCGAGCATCCTGCTCATCTTCTTCTTCATCGCCTTGGTATTC
TTCCTCTACCGGCGCCGCAAAGGCAGTCGCAAAGACGTGACCCTGAGGAAGCTGGATATCAAGGTGGA
GACAGTGAACCGAGAGCCACTTACGATGCATTCTGACCGGGAGGATGACACCGCCAGCGTCTCCACAG
CAACCCGGGTCATGAAGGCCATCTACTCGTCGTTTAAGGATGATGTGGATCTGAAGCAGGACCTGCGC
TGCGACACCATCGACACCCGGGAGGAGTATGAGATGAAGGACCCCACCAATGGCTACTACAACGTGCG
TGCCCATGAAGACCGCCCGTCTTCCAGGGCAGTGCTCTATGCTGACTACCGTGCCCCTGGCCCTGCCC
GCTTCGACGGCCGCCCCTCATCCCGTCTCTCCCACTCCAGCGGCTATGCCCAGCTCAACACCTATAGC
CGGGGCCCTGCCTCTGACTATGGCCCTGAGCCCACACCCCCTGGCCCTGCTGCCCCAGCTGGCACTGA
CACAACCAGCCAGCTGTCCTACGAGAACTATGAGAAGTTCAACTCCCATCCCTTCCCTGGGGCAGCTG
GGTACCCCACCTACCGACTGGGCTACCCCCAGGCCCCACCCTCTGGCCTGGAGCGGACCCCATATGAG
GCGTATGACCCCATTGGCAAGTACGCCACAGCCACTCGATTCTCCTACACCTCCCAGCACTCGGACTA
CGGCCAGCGATTCCAGCAGCGCATGCAGACTCACGTGGTCGACGGC

| | | | |
|---|---|---|---|
| NOV12e, 294385961 | SEQ ID NO: 328 | 763 aa | MW at 84148.5kD |
| Protein Sequence | | | |

TKLMLSLLVWILTLSDTFSQGTQTRFSQEPADQTVVAGQRAVLPCVLLNYSGIVQWTKDGLALGMGQG
LKAWPRYRVVGSADAGQYNLEITDAELSDDASYECQATEAALRSRRAKLTVLIPPEDTRIDGGPVILL
QAGTPHNLTCRAFNAKPAATIIWFRDGTQQEGAVASTELLKDGKRETTVSQLLINPTDLDIGRVFTCR
SMNEAIPSGKETSIELDVHHPPTVTLSIEPQTVQEGERVVFTCQATANPEILGYRWAKGGFLIEDAHE
SRYETNVDYSFFTEPVSCEVHNKVGSTNVSTLVNVHFAPRIVVDPKPTTTDIGSDVTLTCVWVGNPPL
TLTWTKKDSNNVLSNSNQLLLKSVTQADAGTYTCRAIVPRIGVAEREVPLYVNGPPIISSEAVQYAVR
GDGGKVECFIGSTPPPDRIAWAWKENFLEVGTLERYTVERTNSGSGVLSTLTINNVMEADFQTHYNCT
AWNSFGPGTAIIQLEEREVLPVGIIAGATIGASILLIFFFIALVFFLYRRRKGSRKDVTLRKLDIKVE
TVNREPLTMHSDREDDTASVSTATRVMKAIYSSFKDDVDLKQDLRCDTIDTREEYEMKDPTNGYYNVR
AHEDRPSSRAVLYADYRAPGPARFDGRPSSRLSHSSGYAQLNTYSRGPASDYGPEPTPPGPAAPAGTD
TTSQLSYENYEKFNSHPFPGAAGYPTYRLGYPQAPPSGLERTPYEAYDPIGKYATATRFSYTSQHSDY
GQRFQQRMQTHVVDG

| | | |
|---|---|---|
| NOV12f, 294398021 | SEQ ID NO: 329 | 2336 bp |
| DNA Sequence | ORF Start: at 2 | ORF Stop: at 2336 |

CACCAAGCTTATGCTGAGCCTCCTCGTCTGGATCCTCACTCTCTCCGATACTTTCTCCCAAGGGACCC
AGACCCGCTTCAGCCAGGAGCCAGCTGACCAGACGGTGGTGGCTGGACAGCGGGCCGTGCTCCCCTGT
GTGCTGCTCAACTACTCTGGAATTGTGCAATGGACCAAGGACGGGCTGGCCCTGGGCATGGGCCAGGG
CCTCAAAGCCTGGCCACGGTACCGGGTTGTGGGCTCCGCAGACGCTGGGCAGTACAACCTGGAGATCA
CAGATGCTGAGCTCTCTGACGACGCCTCTTACGAGTGCCAGGCCACGGAGGCCGCCCTGCGCTCTCGG
CGGGCCAAACTCACCGTGCTCATCCCCCCAGAGGACACCAGGATTGACGGAGGCCCTGTGATTCTACT
GCAGGCAGGCACCCCCCACAACCTCACATGCCGGGCCTTCAATGCGAAGCCTGCTGCCACCATCATCT
GGTTCCGGGACGGGACGCAGCAGGAGGGCGCTGTGGCCAGCACGGAATTGCTGAAGGATGGGAAGAGG
GAGACCACCGTGAGCCAACTGCTTATTAACCCCACGGACCTGGACATAGGGCGTGTCTTCACTTGCCG
AAGCATGAACGAAGCCATCCCTAGTGGCAAGGAGACTTCCATCGAGCTGGATGTGCACCACCCTCCTA
CAGTGACCCTGTCCATTGAGCCACAGACGGTGCAGGAGGGTGAGCGTGTTGTCTTTACCTGCCAGGCC
ACAGCCAACCCCGAGATCTTGGGCTACAGGTGGGCCAAAGGGGGTTTCTTGATTGAAGACGCCCACGA
GAGTCGCTATGAGACAAATGTGGATTATTCCTTTTTCACGGAGCCTGTGTCTTGTGAGGTTCACAACA
AAGTGGGAAGCACCAATGTCAGCACTTTAGTAAATGTCCACTTTGCTCCCCGGATTGTAGTTGACCCC
AAACCCACAACCACAGACATTGGCTCTGATGTGACCCTTACCTGTGTCTGGGTTGGGAATCCCCCCCT
CACTCTCACCTGGACCAAAAAGGACTCAAATATGGGGCCCAGGCCTCCTGGCTCCCCACCCGAGGCTG
CTCTCTCTGCCCAGGTCCTGAGTAACAGCAACCAGCTGCTGCTGAAGTCGGTGACTCAGGCAGACGCT
GGCACCTACACCTGCCGGGCCATCGTGCCTCGAATCGGAGTGGCTGAGCGGGAGGTGCCGCTCTATGT
GAACGGGCCCCCCATCATCTCCAGTGAGGCAGTGCAGTATGCTGTGAGGGGTGACGGTGGCAAGGTGG
AGTGTTTCATTGGGAGCACACCACCCCCAGACCGCATAGCATGGGCCTGGAAGGAGAACTTCTTGGAG
GTGGGGACCCTGGAACGCTATACAGTGGAGAGGACCAACTCAGGCAGTGGGGTGCTATCCACGCTCAC
CATCAACAATGTCATGGAGGCCGACTTTCAGACTCACTACAACTGCACCGCCTGGAACAGCTTCGGGC
CAGGCACAGCCATCATCCAGCTGGAAGAGCGAGAGGTGTTACCTGTGGGCATCATAGCTGGGGCCACC
ATCGGCGCGAGCATCCTGCTCATCTTCTTCTTCATCGCCTTGGTATTCTTCCTCTACCGGCGCCGCAA
AGGCAGTCGCAAAGACGTGACCCTGAGGAAGCTGGATATCAAGGTGGAGACAGTGAACCGAGAGCCAC
TTACGATGCATTCTGACCGGGAGGATGACACCGCCAGCGTCTCCACAGCAACCCGGGTCATGAAGGCC
ATCTACTCGTCGTTTAAGGATGATGTGGATCTGAAGCAGGACCTGCGCTGCGACACCATCGACACCCG
GGAGGAGTATGAGATGAAGGACCCCACCAATGGCTACTACAACGTGCGTGCCCATGAAGACCGCCCGT
CTTCCAGGGCAGTGCTCTATGCTGACTACCGTGCCCCTGGCCCTGCCCGCTTCGACGGCCGCCCCTCA
TCCCGTCTCTCCCACTCCAGCGGCTATGCCCAGCTCAACACCTATAGCCGGGGCCCTGCCTCTGACTA
TGGCCCTGAGCCCACACCCCCTGGCCCTGCTGCCCCAGCTGGCACTGACACAACCAGCCAGCTGTCCT
ACGAGAACTATGAGAAGTTCAACTCCCATCCCTTCCCTGGGGCAGCTGGGTACCCCACCTACCGACTG
GGCTACCCCCAGGCCCCACCCTCTGGCCTGGAGCGGACCCCATATGAGGCGTATGACCCCATTGGCAA
GTACGCCACAGCCACTCGATTCTCCTACACCTCCCAGCACTCGGACTACGGCCAGCGATTCCAGCAGC
GCATGCAGACTCACGTGGTCGACG

| | | | |
|---|---|---|---|
| NOV12f, 294398021 | SEQ ID NO: 330 | 778 aa | MW at 85605.1kD |
| Protein Sequence | | | |

TKLMLSLLVWILTLSDTFSQGTQTRFSQEPADQTVVAGQPAVLPCVLLNYSGIVQWTKDGLALGMGQG
LKAWPRYRVVGSADAGQYNLEITDAELSDDASYECQATEAALRSRRAKLTVLIPPEDTRIDGGPVILL
QAGTPHNLTCRAFNAKPAATIIWFRDGTQQEGAVASTELLKDGKRETTVSQLLINPTDLDIGRVFTCR
SMNEAIPSGKETSIELDVHHPPTVTLSIEPQTVQEGERVVFTCQATANPEILGYRWAKGGFLIEDAHE
SRYETNVDYSFFTEPVSCEVHNKVGSTNVSTLVNVHFAPRIVVDPKPTTTDIGSDVTLTCVWVGNPPL
TLTWTKKDSNMGPRPPGSPPEAALSAQVLSNSNQLLLKSVTQADAGTYTCRAIVPRIGVAEREVPLYV
NGPPIISSEAVQYAVRGDGGKVECFIGSTPPPDRIAWAWKENFLEVGTLERYTVERTNSGSGVLSTLT
INNVMEADFQTHYNCTAWNSFGPGTAIIQLEEREVLPVGIIAGATIGASILLIFFFIALVFFLYRRRK
GSRKDVTLRKLDIKVETVNREPLTMHSDREDDTASVSTATRVMKAIYSSFKDDVDLKQDLRCDTIDTR
EEYEMKDPTNGYYNVRAHEDRPSSRAVLYADYRAPGPARFDGRPSSRLSHSSGYAQLNTYSRGPASDY
GPEPTPPGPAAPAGTDTTSQLSYENYEKFNSHPFPGAAGYPTYRLGYPQAPPSGLERTPYEAYDPIGK
YATATRFSYTSQHSDYGQRFQQRMQTHVVD

| | | |
|---|---|---|
| NOV12g, CG51373-01 | SEQ ID NO: 331 | 1230 bp |
| DNA Sequence | ORF Start: ATG at 1 | ORF Stop: TAA at 1003 |

ATGCATTTGACTCTGGAAGTCTTAAACCATGGCCCCTTCCCTCTAAACCTTTCCTCCATTGCTTACAA
TCATGGAACTGTGTTTGGCCACTGGAAGAATAACGTCACTCGGGAAACGCTGGTGAAAGTAAAAGATG
CTGAAGATCAGTTGGGTGCACGAGTGGGTTACATCGAACTGGATCTCAACAGCGGGAAGGAAACATTT
CTGGTGAATGAGGAGGCAACGGGCGAGACCTCAGGAGACAATGTTGTTCATTCTAGGAATCTGTCTCA
GACAATCTTCATCACCCGGAAACGATGGGAGGGGACCCAGACCCGCTTCAGCCAGGAGCCAGCTGACC
AGACGGTGGTGGCTGGACAGCGGGCCGTGCTCCCCTGTGTGCTGCTCAACTACTCTGGAATTGTGCAA
TGGACCAAGGACGGGCTGGCCCTGGGCATGGGCCAGGCCCTCAAAGCCTGGCCACGGTACCGGGTTGT
GGGCTCCGCAGACGCTGGGCAGTACAACCTGGAGATCACAGATGCTGAGCTCTCTGACGACGCCTCTT
ACGAGTGCCAGGCCACGGAGGCCGCCCTGCGCTCTCGGCGGGCCAAACTCACCGTGCTCATCCCCCCA
GAGGACACCAGGATTGACGGAGGCCCTGTGATTCTACTGCAGGCAGGCACCCCCCACAACCTCACATG
CCGGGCCTTCAATGCGAAGCCTGCTGCCACCATCATCTGGTTCCGGGACGGGACGCAGCAGGAGGGCG
CTGTGGCCAGCACGGAATTGCTGAAGGATGGGAAGAGGGAGACCACCGTGAGCCAACTGCTTATTAAC
CCCACGGACCTGGACATAGGGCGTGTCTTCACTTGCCGAAGCATGAACGAAGCCATCCCTAGTGGCAA
GGAGACTTCCATCGAGCTGGATGTGCACCGTGAGTGGGCTGGGGGGAGCAGTCTGGAGCAGGGGGGTG
GAAGAAGGGGTGTGTTTGAGAAGCACACTCTTAGTTTGAGAAACACAAACTAAGAGTCCCCCTATGGT
CCCCAGGACAAACGCTTGCCTTCTTCACATCTTTCATTCCCTGGATTGAACCATGGGGACTAAGGGCT
GGTAGAGCATTGGCTGTGGAGTCAGGCAGTCCCCAGGTCTAAACCAGCCTGTTATTAGTCAATGGTTT
ACACTCTCTGGGCCTCGGTTTCCAGTTCTGTATACTGTATATTGCAAAAGATAAAATACTGGCCTACA
GCCCCA

| | | | |
|---|---|---|---|
| NOV12g, CG51373-01 | SEQ ID NO: 332 | 334 aa | MW at 36655.8kD |
| Protein Sequence | | | |

MHLTLEVLNHGPFPLNLSSIAYNHGTVFGHWKNNVTRETLVKVKDAEDQLGARVGYIELDLNSGKETF
LVNEEATGETSGDNVVHSRNLSQTIFITRKRWEGTQTRFSQEPADQTVVAGQRAVLPCVLLNYSGIVQ
WTKDGLALGMGQALKAWPRYRVVGSADAGQYNLEITDAELSDDASYECQATEAALRSRRAKLTVLIPP
EDTRIDGGPVILLQAGTPHNLTCRAFNAKPAATIIWFRDGTQQEGAVASTELLKDGKRETTVSQLLIN
PTDLDIGRVFTCRSMNEAIPSGKETSIELDVHREWAGGSSLEQGGGRRGVFEKHTLSLRNTN

| | | |
|---|---|---|
| NOV12h, CG51373-02 | SEQ ID NO: 333 | 3464 bp |
| DNA Sequence | ORF Start: ATG at 1 | ORF Stop: TAG at 2524 |

ATGCATTTGACTCTGGAAGTCTTAAACCATGGCCCCTTCCCTCTAAACCTTTCCTCCATTGCTTACAA
TCATGGAACTGTGTTTGGCCACTGGAAGAATAACGTCACTCGGGAAACGCTGGTGAAAGTAAAAGATG
CTGAAGATCAGTTGGGTGCACGAGTGGGTTACATCGAACTGGATCTCAACAGCGGGAAGGAAACATTT
CTGGTGAATGAGGAGGCAACGGGCGAGACCTCAGGAGACAATGTTGTTCATTCTAGGAATCTGTCTCA
GACAATCTTCATCACCCGGAAACGATGGGAGGGGACCCAGACCCGCTTCAGCCAGGAGCCAGCTGACC
AGACGGTGGTGGCTGGACAGCGGGCCGTGCTCCCCTGTGTGCTGCTCAACTACTCTGGAATTGTGCAA
TGGACCAAGGACGGGCTGGCCCTGGGCATGGGCCAGGCCCTCAAAGCCTGGCCACGGTACCGGGTTGT
GGGCTCCGCAGACGCTGGGCAGTACAACCTGGAGATCACAGATGCTGAGCTCTCTGACGACGCCTCTT
ACGAGTGCCAGGCCACGGAGGCCGCCCTGCGCTCTCGGCGGGCCAAACTCACCGTGCTCATCCCCCCA
GAGGACACCAGGATTGACGGAGGCCCTGTGATTCTACTGCAGGCAGGCACCCCCCACAACCTCACATG
CCGGGCCTTCAATGCGAAGCCTGCTGCCACCATCATCTGGTTCCGGGACGGGACGCAGCAGGAGGGCG
CTGTGGCCAGCACGGAATTGCTGAAGGATGGGAAGAGGGAGACCACCGTGAGCCAACTGCTTATTAAC
CCCACGGACCTGGACATAGGGCGTGTCTTCACTTGCCGAAGCATGAACGAAGCCATCCCTAGTGGCAA
GGAGACTTCCATCGAGCTGGATGTGCACCACCCTCCTACAGTGACCCTGTCCATTGAGCCACAGACGG
TGCAGGAGGGTGAGCGTGTTGTCTTTACCTGCCAGGCCACAGCCAACCCCGAGATCTTGGGCTACAGG
TGGGCCAAAGGGGGTTTCTTGATTGAAGACGCCCACGAGAGTCGCTATGAGACAAATGTGGATTATTC
CTTTTTCACGGAGCCTGTGTCTTGTGAGGTTCACAACAAAGTGGGAAGCACCAATGTCAGCACTTTAG
TAAATGTCCACTTTGCTCCCCGGATTGTAGTTGACCCCAAACCCACAACCACAGACATTGGCTCTGAT
GTGACCCTTACCTGTGTCTGGGTTGGGAATCCCCCCCTCACTCTCACCTGGACCAAAAAGGACTCAAA
TATGGTCCTGAGTAACAGCAACCAGCTGCTGCTGAAGTCGGTGACTCAGGCAGACGCTGGCACCTACA
CCTGCCGGGCCATCGTGCCTCGAATCGGAGTGGCTGAGCGGGAGGTGCCGCTCTATGTGAACGGGCCC
CCCATCATCTCCAGTGAGGCAGTGCAGTATGCTGTGAGGGGTGACGGTGGCAAGGTGGAGTGTTTCAT
TGGGAGCACACCACCCCCAGACCGCATAGCATGGGCCTGGAAGGAGAACTTCTTGGAGGTGGGGACCC
TGGAACGCTATACAGTGGAGAGGACCAACTCAGGCAGTGGGGTGCTATCCACGCTCACCATCAACAAT
GTCATGGAGGCCGACTTTCAGACTCACTACAACTGCACCGCCTGGAACAGCTTCGGGCCAGGCACAGC
CATCATCCAGCTGGAAGAGCGAGAGGTGTTACCTGTGGGCATCATAGCTGGGGCCACCATCGGCGCGA
GCATCCTGCTCATCTTCTTCTTCATCGCCTTGGTATTCTTCCTCTACCGGCGCCGCAAAGGCAGTCGC
AAAGACGTGACCCTGAGGAAGCTGGATATCAAGGTGGAGACAGTGAACCGAGAGCCACTTACGATGCA
TTCTGACCGGGAGGATGACACCGCCAGCGTCTCCACAGCAACCCGGGTCATGAAGGCCATCTACTCGT
CGTTTAAGGATGATGTGGATCTGAAGCAGGACCTGCGCTGCGACACCATCGACACCCGGGAGGAGTAT
GAGATGAAGGACCCCACCAATGGCTACTACAACGTGCGTGCCCATGAAGACCGCCCGTCTTCCAGGGC
AGTGCTCTATGCTGACTACCGTGCCCCTGGCCCTGCCCGCTTCGACGGCCGCCCCTCATCCCGTCTCT
CCCACTCCAGCGGCTATGCCCAGCTCAACACCTATAGCCGGGGCCCTGCCTCTGACTATGGCCCTGAG
CCCACACCCCCTGGCCCTGCTGCCCCAGCTGGCACTGACACAACCAGCCAGCTGTCCTACGAGAACTA
TGAGAAGTTCAACTCCCATCCCTTCCCTGGGGCAGCTGGGTACCCCACCTACCGACTGGGCTACCCCC
AGGCCCCACCCTCTGGCCTGGAGCGGACCCCATATGAGGCGTATGACCCCATTGGCAAGTACGCCACA
GCCACTCGATTCTCCTACACCTCCCAGCACTCGGACTACGGCCAGCGATTCCAGCAGCGCATGCAGAC
TCACGTGTAGGGGCCAGAGCCTGGCTGGGGCATCTCTGCGGGGCAGAGGAGAAGGCTTTCGCAGCTGT
TCCCTGATATTCAGGGACATTGCTCATTGCTCCCTTCTCGGACCAGCCTTCTTCCTCCCACCATGGCA
GGTGGGGAGCAGGTCTCCCAGAGACACCCCGTCCCGAGGATGGTGCTCTGTGCATGCCCCAGCCTCCT
GGGCCTGCCCTTCCCTCTTCTTCGGGAGGATGTGTCTCTTCTGACCTGCACTCTTGCCTGACCCTAGA
ATGGGGACAGGGAAAGTGAAGGTTAGGGAAAGCAGAGGGGGGCACTTTTTAGCATTCCCTTTCTATCC
CACCCCTCTGATCTCCCATAAGTGGAAATGGGGGTACCCAGGGATGGGCAGGCTTTGGCCTAGGGACA
TGAAGTATGGGAGTGGGTGGCTGTGGCACAGACAGGTGGAAAACGGGATAGCCTGGCCAGTCCCTCTG
TTGTCTGCATTCGTGCCCTGGGTGCCTCTCTCCTTCCTCAGGGTACTGCAGAAGGGAGCGAACAGGGT
ACTGTTCGCTCTTGTCTACAGAACAGCCCTGGCACTGCATTCAAATCCAGTCTTCATTCAGCTGGGAT
CAAAATGCCAGTCACCTTGGCTACCCACTGTGGACAGCTGTCTGTCAGCATGCAGAGGGATCCAGGAA
TCCCCCCGGCAGCACGGCCCGCTTTCCTTCTCCTCCATGCTGGGCCAGCCAGATAAGTCAGGGTCCTG
GTGGAGAAAGAAAGGCTAGGACCATGTCCTCATTGACCCAGATACTGCTGTGTGCTGCACAGCAGTGA
ACCAACACTAGAGGGAGCCACACAAGCCTCCTCTCCCCAGTCTGCCCCACTTCCTGGCTTTAACTCTT
GAGCTGGTTTGGGGAGTGGTGAGGTAGGGGTGGGGGTGCTGTAGGCTCTTTTTCAAAAAAAAAC

| | | | |
|---|---|---|---|
| NOV12h, CG51373-02 | SEQ ID NO: 334 | 841 aa | MW at 92988.2kD |
| Protein Sequence | | | |

MHLTLEVLNHGPFPLNLSSIAYNHGTVFGHWKNNVTRETLVKVKDAEDQLGARVGYIELDLNSGKETF
LVNEEATGETSGDNVVHSRNLSQTIFITRKRWEGTQTRFSQEPADQTVVAGQRAVLPCVLLNYSGIVQ
WTKDGLALGMGQALKAWPRYRVVGSADAGQYNLEITDAELSDDASYECQATEAALRSRRAKLTVLIPP
EDTRIDGGPVILLQAGTPHNLTCRAFNAKPAATIIWFRDGTQQEGAVASTELLKDGKRETTVSQLLIN
PTDLDIGRVFTCRSMNEAIPSGKETSIELDVHHPPTVTLSIEPQTVQEGERVVFTCQATANPEILGYR
WAKGGFLIEDAHESRYETNVDYSFFTEPVSCEVHNKVGSTNVSTLVNVHFAPRIVVDPKPTTTDIGSD
VTLTCVWVGNPPLTLTWTKKDSNMVLSNSNQLLLKSVTQADAGTYTCRAIVPRIGVAEREVPLYVNGP
PIISSEAVQYAVRGDGGKVECFIGSTPPPDRIAWAWKENFLEVGTLERYTVERTNSGSGVLSTLTINN
VMEADFQTHYNCTAWNSFGPGTAIIQLEEREVLPVGIIAGATIGASILLIFFFIALVFFLYRRRKGSR
KDVTLRKLDIKVETVNREPLTMHSDREDDTASVSTATRVMKAIYSSFKDDVDLKQDLRCDTIDTREEY
EMKDPTNGYYNVRAHEDRPSSRAVLYADYRAPGPARFDGRPSSRLSHSSGYAQLNTYSRGPASDYGPE
PTPPGPAAPAGTDTTSQLSYENYEKFNSHPFPGAAGYPTYRLGYPQAPPSGLERTPYEAYDPIGKYAT
ATRFSYTSQHSDYGQRFQQRMQTHV

| | | |
|---|---|---|
| NOV12i, CG51373-03 | SEQ ID NO: 335 | 3379 bp |
| DNA Sequence | ORF Start: ATG at 351 | ORF Stop: TAG at 2439 |

CTCTCCGATACTTTCTCCCAAGGGTCAGCTGCTTCTTCATTCCAAGTGGACAAGGAGCCAGCTGCTCA
CTGTCCTTGAGAGACTTCAGCGAGAGACCAGGGTGTCCAGGCTCCATGCAGGAAAGCCATGCGTATAA
ATTCCACCTCTGAGCCAGGCCTCACCAGCAAGCCCACTCTTAAGCCCTTGACTTGGGCTCCAGGGGCC
ATGGGAAGGAGAAACGGACCCAGACCCGCTTCAGCCAGGAGCCAGCTGACCAGACGGTGGTGGCTGGA
CAGCGGGCCGTGCTCCCCTGTGTGCTGCTCAACTACTCTGGAATTGTGCAATGGACCAAGGACGGGCT
GGCCCTGGGCATGGGCCAGGCCCTCAAAGCCTGGCCACGGTACCGGGTTGTGGGCTCCGCAGACGCTG
GGCAGTACAACCTGGAGATCACAGATGCTGAGCTCTCTGACGACGCCTCTTACGAGTGCCAGGCCACG
GAGGCCGCCCTGCGCTCTCGGCGGGCCAAACTCACCGTGCTCATCCCCCCAGAGGACACCAGGATTGA
CGGAGGCCCTGTGATTCTACTGCAGGCAGGCACCCCCCACAACCTCACATGCCGGGCCTTCAATGCGA
AGCCTGCTGCCACCATCATCTGGTTCCGGGACGGGACGCAGCAGGAGGGCGCTGTGGCCAGCACGGAA
TTGCTGAAGGATGGGAAGAGGGAGACCACCGTGAGCCAACTGCTTATTAACCCCACGGACCTGGACAT
AGGGCGTGTCTTCACTTGCCGAAGCATGAACGAAGCCATCCCTAGTGGCAAGGAGACTTCCATCGAGC
TGGATGTGCACCACCCTCCTACAGTGACCCTGTCCATTGAGCCACAGACGGTGCAGGAGGGTGAGCGT
GTTGTCTTTACCTGCCAGGCCACAGCCAACCCCGAGATCTTGGGCTACAGGTGGGCCAAAGGGGGTTT
CTTGATTGAAGACGCCCACGAGAGTCGCTATGAGACAAATGTGGATTATTCCTTTTTCACGGAGCCTG
TGTCTTGTGAGGTTCACAACAAAGTGGGAAGCACCAATGTCAGCACTTTAGTAAATGTCCACTTTGCT
CCCCGGATTGTAGTTGACCCCAAACCCACAACCACAGACATTGGCTCTGATGTGACCCTTACCTGTGT
CTGGGTTGGGAATCCCCCCCTCACTCTCACCTGGACCAAAAAGGACTCAAATATGGTCCTGAGTAACA
GCAACCAGCTGCTGCTGAAGTCGGTGACTCAGGCAGACGCTGGCACCTACACCTGCCGGGCCATCGTG
CCTCGAATCGGAGTGGCTGAGCGGGAGGTGCCGCTCTATGTGAACGGGCCCCCCATCATCTCCAGTGA
GGCAGTGCAGTATGCTGTGAGGGGTGACGGTGGCAAGGTGGAGTGTTTCATTGGGAGCACACCACCCC
CAGACCGCATAGCATGGGCCTGGAAGGAGAACTTCTTGGAGGTGGGGACCCTGGAACGCTATACAGTG
GAGAGGACCAACTCAGGCAGTGGGGTGCTATCCACGCTCACCATCAACAATGTCATGGAGGCCGACTT
TCAGACTCACTACAACTGCACCGCCTGGAACAGCTTCGGGCCAGGCACAGCCATCATCCAGCTGGAAG
AGCGAGAGGTGTTACCTGTGGGCATCATAGCTGGGGCCACCATCGGCGCGAGCATCCTGCTCATCTTC
TTCTTCATCGCCTTGGTATTCTTCCTCTACCGGCGCCGCAAAGGCAGTCGCAAAGACGTGACCCTGAG
GAAGCTGGATATCAAGGTGGAGACAGTGAACCGAGAGCCACTTACGATGCATTCTGACCGGGAGGATG
ACACCGCCAGCGTCTCCACAGCAACCCGGGTCATGAAGGCCATCTACTCGTCGTTTAAGGATGATGTG
GATCTGAAGCAGGACCTGCGCTGCGACACCATCGACACCCGGGAGGAGTATGAGATGAAGGACCCCAC
CAATGGCTACTACAACGTGCGTGCCCATGAAGACCGCCCGTCTTCCAGGGCAGTGCTCTATGCTGACT
ACCGTGCCCCTGGCCCTGCCCGCTTCGACGGCCGCCCCTCATCCCGTCTCTCCCACTCCAGCGGCTAT
GCCCAGCTCAACACCTATAGCCGGGGCCCTGCCTCTGACTATGGCCCTGAGCCCACACCCCCTGGCCC
TGCTGCCCCAGCTGGCACTGACACAACCAGCCAGCTGTCCTACGAGAACTATGAGAAGTTCAACTCCC
ATCCCTTCCCTGGGGCAGCTGGGTACCCCACCTACCGACTGGGCTACCCCCAGGCCCCACCCTCTGGC
CTGGAGCGGACCCCATATGAGGCGTATGACCCCATTGGCAAGTACGCCACAGCCACTCGATTCTCCTA
CACCTCCCAGCACTCGGACTACGGCCAGCGATTCCAGCAGCGCATGCAGACTCACGTGTAGGGGCCAG
AGCCTGGCTGGGGCATCTCTGCGGGGCAGAGGAGAAGGCTTTCGCAGCTGTTCCCTGATATTCAGGGA
CATTGCTCATTGCTCCCTTCTCGGACCAGCCTTCTTCCTCCCACCATGGCAGGTGGGGAGCAGGTCTC
CCAGAGACACCCCGTCCCGAGGATGGTGCTCTGTGCATGCCCCAGCCTCCTGGGCCTGCCCTTCCCTC
TTCTTCGGGAGGATGTGTCTCTTCTGACCTGCACTCTTGCCTGACCCTAGAATGGGGACAGGGAAAGT
GAAGGTTAGGGAAAGCAGAGGGGGGCACTTTTTAGCATTCCCTTTCTATCCCACCCCTCTGATCTCCC
ATAAGTGGAAATGGGGGTACCCAGGGATGGGCAGGCTTTGGCCTAGGGACATGAAGTATGGGAGTGGG
TGGCTGTGGCACAGACAGGTGGAAAACGGGATAGCCTGGCCAGTCCCTCTGTTGTCTGCATTCGTGCC
CTGGGTGCCTCTCTCCTTCCTCAGGGTACTGCAGAAGGGAGCGAACAGGGTACTGTTCGCTCTTGTCT
ACAGAACAGCCCTGGCACTGCATTCAAATCCAGTCTTCATTCAGCTGGGATCAAAATGCCAGTCACCT
TGGCTACCCACTGTGGACAGCTGTCTGTCAGCATGCAGAGGGATCCAGGAATCCCCCCGGCAGCACGC
CCCGCTTTCCTTCTCCTCCATGCTGGGCCAGCCAGATAAGTCAGGGTCCTGGTGGAGAAAGAAAGGCT
AGGACCATGTCCTCATTGACCCAGATACTGCTGTGTGCTGCACAGCAGTGAACCAACACTAGAGGGAG
CCACACAAGCCTCCTCTCCCCAGTCTGCCCCACTTCCTGGCTTTAACTCTTGAGCTGGTTTGGGGAGT
GGTGAGGTAGGGGTGGGGGTGCTGTAGGCTCTTTTTCAAAAAAAAAC

| | | | |
|---|---|---|---|
| NOV12i, CG51373-03 | SEQ ID NO: 336 | 696 aa | MW at 76928.3kD |
| Protein Sequence | | | |

MGQALKAWPRYRVVGSADAGQYNLEITDAELSDDASYECQATEAALRSRRAKLTVLIPPEDTRIDGGP
VILLQAGTPHNLTCRAFNAKPAATIIWFRDGTQQEGAVASTELLKDGKRETTVSQLLINPTDLDIGRV
FTCRSMNEAIPSGKETSIELDVHHPPTVTLSIEPQTVQEGERVVFTCQATANPEILGYRWAKGGFLIE
DAHESRYETNVDYSFFTEPVSCEVHNKVGSTNVSTLVNVHFAPRIVVDPKPTTTDIGSDVTLTCVWVG
NPPLTLTWTKKDSNMVLSNSNQLLLKSVTQADAGTYTCRAIVPRIGVAEREVPLYVNGPPIISSEAVQ
YAVRGDGGKVECFIGSTPPPDRIAWAWKENFLEVGTLERYTVERTNSGSGVLSTLTINNVMEADFQTH
YNCTAWNSFGPGTAIIQLEEREVLPVGIIAGATIGASILLIFFFIALVFFLYRRRKGSRKDVTLRKLD
IKVETVNREPLTMHSDREDDTASVSTATRVMKAIYSSFKDDVDLKQDLRCDTIDTREEYEMKDPTNGY
YNVRAHEDRPSSRAVLYADYRAPGPARFDQRPSSRLSHSSGYAQLNTYSRGPASDYGPEPTPPGPAAP
AGTDTTSQLSYENYEKFNSHPFPGAAGYPTYRLGYPQAPPSGLERTPYEAYDPIGKYATATRFSYTSQ
HSDYGQRFQQRMQTHV

| | | |
|---|---|---|
| NOV12i, CG51373-04 | SEQ ID NO: 337 | 1145 bp |
| DNA Sequence | ORF Start: ATG at 351 | ORF Stop: TAA at 918 |

CTCTCCGATACTTTCTCCCAAGGGTCAGCTGCTTCTTCATTCCAAGTGGACAAGGAGCCAGCTGCTCA
CTGTCCTTGAGAGACTTCAGCGAGAGACCAGGGTGTCCAGGCTCCATGCAGGAAAGCCATGCGTATAA
ATTCCACCTCTGAGCCAGGCCTCACCAGCAAGCCCACTCTTAAGCCCTTGACTTGGGCTCCAGGGGCC
ATGGGAAGGAGAAACGGACCCAGACCCGCTTCAGCCAGGAGCCAGCTGACCAGACGGTGGTGGCTGGA
CAGCGGGCCGTGCTCCCCTGTGTGCTGCTCAACTACTCTGGAATTGTGCAATGGACCAAGGACGGGCT
GGCCCTGGGCATGGGCCAGGCCCTCAAAGCCTGGCCACGGTACCGGGTTGTGGGCTCCGCAGACGCTG
GGCAGTACAACCTGGAGATCACAGATGCTGAGCTCTCTGACGACGCCTCTTACGAGTGCCAGGCCACG
GAGGCCGCCCTGCGCTCTCGGCGGGCCAAACTCACCGTGCTCATCCCCCCAGAGGACACCAGGATTGA
CGGAGGCCCTGTGATTCTACTGCAGGCAGGCACCCCCCACAACCTCACATGCCGGGCCTTCAATGCGA
AGCCTGCTGCCACCATCATCTGGTTCCGGGACGGGACGCAGCAGGAGGGCGCTGTGGCCAGCACGGAA
TTGCTGAAGGATGGGAAGAGGGAGACCACCGTGAGCCAACTGCTTATTAACCCCACGGACCTGGACAT
AGGGCGTGTCTTCACTTGCCGAAGCATGAACGAAGCCATCCCTAGTGGCAAGGAGACTTCCATCGAGC
TGGATGTGCACCGTGAGTGGGCTGGGGGGAGCAGTCTGGAGCAGGGGGGTGGAAGAAGGGGTGTGTTT
GAGAAGCACACTCTTAGTTTGAGAAACACAAACTAAGAGTCCCCCTATGGTCCCCAGGACAAACGCTT
GCCTTCTTCACATCTTTCATTCCCTGGATTGAACCATGGGGACTAAGGGCTGGTAGAGCATTGGCTGT
GGAGTCAGGCAGTCCCCAGGTCTAAACCAGCCTGTTATTAGTCAATGGTTTACACTCTCTGGGCCTCG
GTTTCCAGTTCTGTATACTGTATATTGCAAAAGATAAAATACTGGCCTACAGCCCCA

| | | | |
|---|---|---|---|
| NOV12j, CG51373-04 | SEQ ID NO: 338 | 189 aa | MW at 20595.8kD |
| Protein Sequence | | | |

MGQALKAWPRYRVVGSADAGQYNLEITDAELSDDASYECQATEAALRSRRAKLTVLIPPEDTRIDGGP
VILLQAGTPHNLTCRAFNAKPAATIIWFRDGTQQEGAVASTELLKDGKRETTVSQLLINPTDLDIGRV
FTCRSMNEAIPSGKETSIELDVHREWAGGSSLEQGGGRRGVFEKHTLSLRNTN

| | | |
|---|---|---|
| NOV12k, CG51373-05 | SEQ ID NO: 339 | 1148 bp |
| DNA Sequence | ORF Start: ATG at 351 | ORF Stop: TAA at 918 |

CTCTCCGATACTTTCTCCCAAGGGTCAGCTGCTTCTTCATTCCAAGTGGACAAGGAGCCAGCTGCTCA
CTGTCCTTGAGAGACTTCAGCGAGAGACCAGGGTGTCCAGGCTCCATGCAGGAAAGCCATGCGTATAA
ATTCCACCTCTGAGCCAGGCCTCACCAGCAAGCCCACTCTTAAGCCCTTGACTTGGGCTCCAGGGGCC
ATGGGAAGGAGAAACGGACCCAGACCCGCTTCAGCCAGGAGCCAGCTGACCAGACGGTGGTGGCTGGA
CAGCGGGCCGTGCTCCCCTGTGTGCTGCTCAACTACTCTGGAATTGTGCAATGGACCAAGGACGGGCT
GGCCCTGGGCATGGGCCAGGCCCTCAAAGCCTGGCCACGGTACCGGGTTGTGGGCTCCGCAGACGCTG
GGCAGTACAACCTGGAGATCACAGATGCTGAGCTCTCTGACGACGCCTCTTACGAGTGCCAGGCCACG
GAGGCCGCCCTGCGCTCTCGGCGGGCCAAACTCACCGTGCTCATCCCCCCAGAGGACACCAGGATTGA
CGGAGGCCCTGTGATTCTACTGCAGGCAGGCACCCCCCACAACCTCACATGCCGGGCCTTCAATGCGA
AGCCTGCTGCCACCATCATCTGGTTCCGGGACGGGACGCAGCAGGAGGGCGCTGTGGCCAGCACGGAA
TTGCTGAAGGATGGGAAGAGGGAGACCACCGTGAGCCAACTGCTTATTAACCCCACGGACCTGGACAT
AGGGCGTGTCTTCACTTGCCGAAGCATGAACGAAGCCATCCCTAGTGGCAAGGAGACTTCCATCGAGC
TGGATGTGCACCGTGAGTGGGCTGGGGGGAGCAGTCTGGAGCAGGGGGGTGGAAGAAGGGGTGTGTTT
GAGAAGCACACTCTTAGTTTGAGAAACACAAACTAAGAGTCCCCCTATGGTCCCCAGGACAAACGCTT
GCCTTCTTCACATCTTTCATTCCCTGGATTGAACCATGGGGACTAAGGGCTGGTAGAGCATTGGCTGT
GGAGTCAGGCAGTCCCCAGGTCTAAACCAGCCTGTTATTAGTCAATGGTTTACACTCTCTGGGCCTCG
GTTTCCAGTTCTGTATACTGTATATTGCAAAAGATAAAATACTGGCCTACAGCCCCAAAA

| | | | |
|---|---|---|---|
| NOV12k, CG51373-05 | SEQ ID NO: 340 | 189 aa | MW at 20595.8kD |
| Protein Sequence | | | |

MGQALKAWPRYRVVGSADAGQYNLEITDAELSDDASYECQATEAALRSRRAKLTVLIPPEDTRIDGGP
VILLQAGTPHNLTCRAFNAKPAATIIWFRDGTQQEGAVASTELLKDGKRETTVSQLLINPTDLDIGRV
FTCRSMNEAIPSGKETSIELDVHREWAGGSSLEQGGGRRGVFEKHTLSLRNTN

| | | |
|---|---|---|
| NOV12l, CG51373-06 | SEQ ID NO: 341 | 1148 bp |
| DNA Sequence | ORF Start: ATG at 351 | ORF Stop: TAA at 918 |

CTCTCCGATACTTTCTCCCAAGGGTCAGCTGCTTCTTCATTCCAAGTGGACAAGGAGCCAGCTGCTCA
CTGTCCTTGAGAGACTTCAGCGAGAGACCAGGGTGTCCAGGCTCCATGCAGGAAAGCCATGCGTATAA
ATTCCACCTCTGAGCCAGGCCTCACCAGCAAGCCCACTCTTAAGCCCTTGACTTGGGCTCCAGGGGCC
ATGGGAAGGAGAAACGGACCCAGACCCGCTTCAGCCAGGAGCCAGCTGACCAGACGGTGGTGGCTGGA
CAGCGGGCCGTGCTCCCCTGTGTGCTGCTCAACTACTCTGGAATTGTGCAATGGACCAAGGACGGGCT
GGCCCTGGGCATGGGCCAGGCCCTCAAAGCCTGGCCACGGTACCGGGTTGTGGGCTCCGCAGACGCTG
GGCAGTACAACCTGGAGATCACAGATGCTGAGCTCTCTGACGACGCCTCTTACGAGTGCCAGGCCACG
GAGGCCGCCCTGCGCTCTCGGCGGGCCAAACTCACCGTGCTCATCCCCCCAGAGGACACCAGGATTGA
CGGAGGCCCTGTGATTCTACTGCAGGCAGGCACCCCCCACAACCTCACATGCCGGGCCTTCAATGCGA
AGCCTGCTGCCACCATCATCTGGTTCCGGGACGGGACGCAGCAGGAGGGCGCTGTGGCCAGCACGGAA
TTGCTGAAGGATGGGAAGAGGGAGACCACCGTGAGCCAACTGCTTATTAACCCCACGGACCTGGACAT
AGGGCGTGTCTTCACTTGCCGAAGCATGAACGAAGCCATCCCTAGTGGCAAGGAGACTTCCATCGAGC
TGGATGTGCACCGTGAGTGGGCTGGGGGGAGCAGTCTGGAGCAGGGGGGTGGAAGAAGGGGTGTGTTT
GAGAAGCACACTCTTAGTTTGAGAAACACAAACTAAGAGTCCCCCTATGGTCCCCAGGACAAACGCTT
GCCTTCTTCACATCTTTCATTCCCTGGATTGAACCATGGGGACTAAGGGCTGGTAGAGCATTGGCTGT
GGAGTCAGGCAGTCCCCAGGTCTAAACCAGCCTGTTATTAGTCAATGGTTTACACTCTCTGGGCCTCG
GTTTCCAGTTCTGTATACTGTATATTGCAAAAGATAAAATACTGGCCTACAGCCCCAAAA

| | | | |
|---|---|---|---|
| NOV12l, CG51373-06 | SEQ ID NO: 342 | 189 aa | MW at 20595.8kD |
| Protein Sequence | | | |

MGQALKAWPRYRVVGSADAGQYNLEITDAELSDDASYECQATEAALRSRRAKLTVLIPPEDTRIDGGP
VILLQAGTPHNLTCRAFNAKPAATIIWFRDGTQQEGAVASTELLKDGKRETTVSQLLINPTDLDIGRV
FTCRSMNEAIPSGKETSIELDVHREWAGGSSLEQGGGRRGVFEKHTLSLRNTN

| | | |
|---|---|---|
| NOV12m, CG51373-07 | SEQ ID NO: 343 | 1407 bp |
| DNA Sequence | ORF Start: at 1 | ORF Stop: end of sequence |

CGCTTCAGCCAGGAGCCAGCTGACCAGACGGTGGTGGCTGGACAGCGGGCCGTGCTCCCCTGTGTGCT
GCTCAACTACTCTGGAATTGTGCAATGGACCAAGGACGGGCTGGCCCTGGGCATGGGCCAGGCCCTCA
AAGCCTGGCCACGGTACCGGGTTGTGGGCTCCGCAGACGCTGGGCAGTACAACCTGGAGATCACAGAT
GCTGAGCTCTCTGACGACGCCTCTTACGAGTGCCAGGCCACGGAGGCCGCCCTGCGCTCTCGGCGGGC
CAAACTCACCGTGCTCATCCCCCCAGAGGACACCAGGATTGACGGAGGCCCTGTGATTCTACTGCAGG
CAGGCACCCCCCACAACCTCACATGCCGGGCCTTCAATGCGAAGCCTGCTGCCACCATCATCTGGTTC
CGGGACGGGACGCAGCAGGAGGGCGCTGTGGCCAGCACGGAATTGCTGAAGGATGGGAAGAGGGAGAC
CACCGTGAGCCAACTGCTTATTAACCCCACGGACCTGGACATAGGGCGTGTCTTCACTTGCCGAAGCA
TGAACGAAGCCATCCCTAGTGGCAAGGAGACTTCCATCGAGCTGGATGTGCACCACCCTCCTACAGTG
ACCCTGTCCATTGAGCCACAGACGGTGCAGGAGGGTGAGCGTGTTGTCTTTACCTGCCAGGCCACAGC
CAACCCCGAGATCTTGGGCTACAGGTGGGCCAAAGGGGGTTTCTTGATTGAAGACGCCCACGAGAGTC
GCTATGAGACAAATGTGGATTATTCCTTTTTCACGGAGCCTGTGTCTTGTGAGGTTCACAACAAAGTG
GGAAGCACCAATGTCAGCACTTTAGTAAATGTCCACTTTGCTCCCCGGATTGTAGTTGACCCCAAACC
CACAACCACAGACATTGGCTCTGATGTGACCCTTACCTGTGTCTGGGTTGGGAATCCCCCCCTCACTC
TCACCTGGACCAAAAAGGACTCAAATATGGTCCTGAGTAACAGCAACCAGCTGCTGCTGAAGTCGGTG
ACTCAGGCAGACGCTGGCACCTACACCTGCCGGGCCATCGTGCCTCGAATCGGAGTGGCTGAGCGGGA
GGTGCCGCTCTATGTGAACGGGCCCCCCATCATCTCCAGTGAGGCAGTGCAGTATGCTGTGAGGGGTG
ACGGTGGCAAGGTGGAGTGTTTCATTGGGAGCACACCACCCCCAGACCGCATAGCATGGGCCTGGAAG
GAGAACTTCTTGGAGGTGGGGACCCTGGAACGCTATACAGTGGAGAGGACCAACTCAGGCAGTGGGGT
GCTATCCACGCTCACCATCAACAATGTCATGGAGGCCGACTTTCAGACTCACTACAACTGCACCGCCT
GGAACAGCTTCGGGCCAGGCACAGCCATCATCCAGCTGGAAGAGCGA

| | | | |
|---|---|---|---|
| NOV12m, CG51373-07 | SEQ ID NO: 344 | 469 aa | MW at 51246.2kD |
| Protein Sequence | | | |

RFSQEPADQTVVAGQRAVLPCVLLNYSGIVQWTKDGLALGMGQALKAWPRYRVVGSADAGQYNLEITD
AELSDDASYECQATEAALRSRRAKLTVLIPPEDTRIDGGPVILLQAGTPHNLTCRAFNAKPAATIIWF
RDGTQQEGAVASTELLKDGKRETTVSQLLINPTDLDIGRVFTCRSMNEAIPSGKETSIELDVHHPPTV
TLSIEPQTVQEGERVVFTCQATANPEILGYRWAKGGFLIEDAHESRYETNVDYSFFTEPVSCEVHNKV
GSTNVSTLVNVHFAPRIVVDPKPTTTDIGSDVTLTCVWVGNPPLTLTWTKKDSNMVLSNSNQLLLKSV
TQADAGTYTCRAIVPRIGVAEREVPLYVNGPPIISSEAVQYAVRGDGGKVECFIGSTPPPDRIAWAWK
ENFLEVGTLERYTVERTNSGSGVLSTLTINNVMEADFQTHYNCTAWNSFGPGTAIIQLEER

| | | |
|---|---|---|
| NOV12n, CG51373-09 | SEQ ID NO: 345 | 2128 bp |
| DNA Sequence | ORF Start: ATG at 31 | ORF Stop: TAG at 2119 |

CAATGGACCAAGGACGGGCTGGCCCTGGGCATGGGCCAGGGCCTCAAAGCCTGGCCACGGTACCGGGT
TGTGGGCTCCGCAGACGCTGGGCAGTACAACCTGGAGATCACAGATGCTGAGCTCTCTGACGACGCCT
CTTACGAGTGCCAGGCCACGGAGGCCGCCCTGCGCTCTCGGCGGGCCAAACTCACCGTGCTCATCCCC
CCAGAGGACACCAGGATTGACGGAGGCCCTGTGATTCTACTGCAGGCAGGCACCCCCCACAACCTCAC
ATGCCGGGCCTTCAATGCGAAGCCTGCTGCCACCATCATCTGGTTCCGGGACGGGACGCAGCAGGAGG
GCGCTGTGGCCAGCACGGAATTGCTGAAGGATGGGAAGAGGGAGACCACCGTGAGCCAACTGCTTATT
AACCCCACGGACCTGGACATAGGGCGTGTCTTCACTTGCCGAAGCATGAACGAAGCCATCCCTAGTGG
CAAGGAGACTTCCATCGAGCTGGATGTGCACCACCCTCCTACAGTGACCCTGTCCATTGAGCCACAGA
CGGTGCAGGAGGGTGAGCGTGTTGTCTTTACCTGCCAGGCCACAGCCAACCCCGAGATCTTGGACTAC
AGGTGGGCCAAAGGGGGTTTCTTGATTGAAGACGCCCACGAGAGTCGCTATGAGACAAATGTGGATTA
TTCCTTTTTCACGGAGCCTGTGTCTTGTGAGGTTCACAACAAAGTGGGAAGCACCAATGTCAGCACTT
TAGTAAATGTCCACTTTGCTCCCCGGATTGTAGTTGACCCCAAACCCACAACCACAGACATTGGCTCT
GATGTGACCCTTACCTGTGTCTGGGTTGGGAATCCCCCCCTCACTCTCACCTGGACCAAAAAGGACTC
AAATATGGTCCTGAGTAACAGCAACCAGCTGCTGCTGAAGTCGGTGACTCAGGCAGACGCTGGCACCT
ACACCTGCCGGGCCATCGTGCCTCGAATCGGAGTGGCTGAGCGGGAGGTGCCGCTCTATGTGAACGGG
CCCCCCATCATCTCCAGTGAGGCAGTGCAGTATGCTGTGAGGGGTGACGGTGGCAAGGTGGAGTGTTT
CATTGGGAGCACACCACCCCCAGACCGCATAGCATGGGCCTGGAAGGAGAACTTCTTGGAGGTGGGGA
CCCTGGAACGCTATACAGTGGAGAGGACCAACTCAGGCAGTGGGGTGCTATCCACGCTCACCATCAAC
AATGTCATGGAGGCCGACTTTCAGACTCACTACAACTGCACCGCCTGGAACAGCTTCGGGCCAGGCAC
AGCCATCATCCAGCTGGAAGAGCGAGAGGTGTTACCTGTGGGCATCATAGCTGGGGCCACCATCGGCG
CGAGCATCCTGCTCATCTTCTTCTTCATCGCCTTGGTATTCTTCCTCTACCGGCGCCGCAAAGGCAGT
CGCAAAGACGTGACCCTGAGGAAGCTGGATATCAAGGTGGAGACAGTGAACCGAGAGCCACTTACGAT
GCATTCTGACCGGGAGGATGACACCGCCAGCGTCTCCACAGCAACCCGGGTCATGAAGGCCATCTACT
CGTCGTTTATGGATGATGTGGATCTGAAGCAGGACCTGCGCTGCGACACCATCGACACCCGGGAGGAG
TATGAGATGAAGGACCCCACCAATGGCTACTACAACGTGCGTGCCCATGAAGACCGCCCGTCTTCCAG
GGCAGTGCTCTATGCTGACTACCGTGCCCCTGGCCCTGCCCGCTTCGACGGCCGCCCCTCATCCCGTC
TCTCCCACTCCAGCGGCTATGCCCAGCTCAACACCTATAGCCGGGGCCCTGCCTCTGACTATGGCCCT
GAGCCCACACCCCCTGGCCCTGCTGCCCCAGCTGGCACTGACACAACCAGCCAGCTGTCCTACGAGAA
CTATGAGAAGTTCAACTCCCATCCCTTCCCTGGGGCAGCTGGGTACCCCACCTACCGACTGGGCTACC
CCCAGGCCCCACCCTCTGGCCTGGAGCGGACCCCATATGAGGCGTATGACCCCATTGGCAAGTACGCC
ACAGCCACTCGATTCTCCTACACCTCCCAGCACTCGGACTACGGCCAGCGATTCCAGCAGCGCATGCA
GACTCACGTGTAGGGGCCAG

| | | | |
|---|---|---|---|
| NOV12n, CG51373-09 | SEQ ID NO: 346 | 696 aa | MW at 76975.3kD |
| Protein Sequence | | | |

MGQGLKAWPRYRVVGSADAGQYNLEITDAELSDDASYECQATEAALRSRRAKLTVLIPPEDTRIDGGP
VILLQAGTPHNLTCRAFNAKPAATIIWFRDGTQQEGAVASTELLKDGKRETTVSQLLINPTDLDIGRV
FTCRSMNEAIPSGKETSIELDVHHPPTVTLSIEPQTVQEGERVVFTCQATANPEILDYRWAKGGFLIE
DAHESRYETNVDYSFFTEPVSCEVHNKVGSTNVSTLVNVHFAPRIVVDPKPTTTDIGSDVTLTCVWVG
NPPLTLTWTKKDSNMVLSNSNQLLLKSVTQADAGTYTCRAIVPRIGVAEREVPLYVNGPPIISSEAVQ
YAVRGDGGKVECFIGSTPPPDRIAWAWKENFLEVGTLERYTVERTNSGSGVLSTLTINNVMEADFQTH
YNCTAWNSFGPGTAIIQLEEREVLPVGIIAGATIGASILLIFFFIALVFFLYRRRKGSRKDVTLRKLD
IKVETVNREPLTMHSDREDDTASVSTATRVMKAIYSSFMDDVDLKQDLRCDTIDTREEYEMKDPTNGY
YNVRAHEDRPSSRAVLYADYRAPGPARFDGRPSSRLSHSSGYAQLNTYSRGPASDYGPEPTPPGPAAP
AGTDTTSQLSYENYEKFNSHPFPGAAGYPTYRLGYPQAPPSGLERTPYEAYDPIGKYATATRFSYTSQ
HSDYGQRFQQRMQTNV

| | | |
|---|---|---|
| NOV12o, CG51373-12 | SEQ ID NO: 347 | 2336 bp |
| DNA Sequence | ORF Start: ATG at 11 | ORF Stop: at 2330 |

CACCAAGCTTATGCTGAGCCTCCTCGTCTGGATCCTCACTCTCTCCGATACTTTCTCCCAAGGGACCC
AGACCCGCTTCAGCCAGGAGCCAGCTGACCAGACGGTGGTGGCTGGACAGCGGGCCGTGCTCCCCTGT
GTGCTGCTCAACTACTCTGGAATTGTGCAATGGACCAAGGACGGGCTGGCCCTGGGCATGGGCCAGGG
CCTCAAAGCCTGGCCACGGTACCGGGTTGTGGGCTCCGCAGACGCTGGGCAGTACAACCTGGAGATCA
CAGATGCTGAGCTCTCTGACGACGCCTCTTACGAGTGCCAGGCCACGGAGGCCGCCCTGCGCTCTCGG
CGGGCCAAACTCACCGTGCTCATCCCCCCAGAGGACACCAGGATTGACGGAGGCCCTGTGATTCTACT
GCAGGCAGGCACCCCCCACAACCTCACATGCCGGGCCTTCAATGCGAAGCCTGCTGCCACCATCATCT
GGTTCCGGGACGGGACGCAGCAGGAGGGCGCTGTGGCCAGCACGGAATTGCTGAAGGATGGGAAGAGG
GAGACCACCGTGAGCCAACTGCTTATTAACCCCACGGACCTGGACATAGGGCGTGTCTTCACTTGCCG
AAGCATGAACGAAGCCATCCCTAGTGGCAAGGAGACTTCCATCGAGCTGGATGTGCACCACCCTCCTA
CAGTGACCCTGTCCATTGAGCCACAGACGGTGCAGGAGGGTGAGCGTGTTGTCTTTACCTGCCAGGCC
ACAGCCAACCCCGAGATCTTGGGCTACAGGTGGGCCAAAGGGGGTTTCTTGATTGAAGACGCCCACGA
GAGTCGCTATGAGACAAATGTGGATTATTCCTTTTTCACGGAGCCTGTGTCTTGTGAGGTTCACAACA
AAGTGGGAAGCACCAATGTCAGCACTTTAGTAAATGTCCACTTTGCTCCCCGGATTGTAGTTGACCCC
AAACCCACAACCACAGACATTGGCTCTGATGTGACCCTTACCTGTGTCTGGGTTGGGAATCCCCCCCT
CACTCTCACCTGGACCAAAAAGGACTCAAATATGGGGCCCAGGCCTCCTGGCTCCCCACCCGAGGCTG
CTCTCTCTGCCCAGGTCCTGAGTAACAGCAACCAGCTGCTGCTGAAGTCGGTGACTCAGGCAGACGCT
GGCACCTACACCTGCCGGGCCATCGTGCCTCGAATCGGAGTGGCTGAGCGGGAGGTGCCGCTCTATGT
GAACGGGCCCCCCATCATCTCCAGTGAGGCAGTGCAGTATGCTGTGAGGGGTGACGGTGGCAAGGTGG
AGTGTTTCATTGGGAGCACACCACCCCCAGACCGCATAGCATGGGCCTGGAAGGAGAACTTCTTGGAG
GTGGGGACCCTGGAACGCTATACAGTGGAGAGGACCAACTCAGGCAGTGGGGTGCTATCCACGCTCAC
CATCAACAATGTCATGGAGGCCGACTTTCAGACTCACTACAACTGCACCGCCTGGAACAGCTTCGGGC
CAGGCACAGCCATCATCCAGCTGGAAGAGCGAGAGGTGTTACCTGTGGGCATCATAGCTGGGGCCACC
ATCGGCGCGAGCATCCTGCTCATCTTCTTCTTCATCGCCTTGGTATTCTTCCTCTACCGGCGCCGCAA
AGGCAGTCGCAAAGACGTGACCCTGAGGAAGCTGGATATCAAGGTGGAGACAGTGAACCGAGAGCCAC
TTACGATGCATTCTGACCGGGAGGATGACACCGCCAGCGTCTCCACAGCAACCCGGGTCATGAAGGCC
ATCTACTCGTCGTTTAAGGATGATGTGGATCTGAAGCAGGACCTGCGCTGCGACACCATCGACACCCG
GGAGGAGTATGAGATGAAGGACCCCACCAATGGCTACTACAACGTGCGTGCCCATGAAGACCGCCCGT
CTTCCAGGGCAGTGCTCTATGCTGACTACCGTGCCCCTGGCCCTGCCCGCTTCGACGGCCGCCCCTCA
TCCCGTCTCTCCCACTCCAGCGGCTATGCCCAGCTCAACACCTATAGCCGGGGCCCTGCCTCTGACTA
TGGCCCTGAGCCCACACCCCCTGGCCCTGCTGCCCCAGCTGGCACTGACACAACCAGCCAGCTGTCCT
ACGAGAACTATGAGAAGTTCAACTCCCATCCCTTCCCTGGGGCAGCTGGGTACCCCACCTACCGACTG
GGCTACCCCCAGGCCCCACCCTCTGGCCTGGAGCGGACCCCATATGAGGCGTATGACCCCATTGGCAA
GTACGCCACAGCCACTCGATTCTCCTACACCTCCCAGCACTCGGACTACGGCCAGCGATTCCAGCAGC
GCATGCAGACTCACGTGGTCGACG

| | | | |
|---|---|---|---|
| NOV12o, CG51373-12 | SEQ ID NO: 348 | 773 aa | MW at 85048.5kD |
| Protein Sequence | | | |

MLSLLVWILTLSDTFSQGTQTRFSQEPADQTVVAGQRAVLPCVLLNYSGIVQWTKDGLALGMGQGLKA
WPRYRVVGSADAGQYNLEITDAELSDDASYECQATEAALRSRHAKLTVLIPPEDTRIDGGPVILLQAG
TPHNLTCRAFNAKPAATIIWFRDGTQQEGAVASTELLKDGKRETTVSQLLINPTDLDIGRVFTCRSMN
EAIPSGKETSIELDVHHPPTVTLSIEPQTVQEGERVVFTCQATANPEILGYRWAKGGFLIEDAHESRY
ETNVDYSFFTEPVSCEVHNKVGSTNVSTLVNVHFAPRIVVDPKPTTTDTGSDVTLTCVWVGNPPLTLT
WTKKDSNMGPRPPGSPPEAALSAQVLSNSNQLLLKSVTQADAGTYTCRAIVPRIGVAEREVPLYVNGP
PIISSEAVQYAVRGDGGKVECFIGSTPPPDRIAWAWKENFLEVGTLERYTVERTNSGSGVLSTLTINN
VMEADFQTHYNCTAWNSFGPGTAIIQLEEREVLPVGIIAGATIGASILLIFFFIALVFFLYRPRKGSR
KDVTLRXLDIKVETVNREPLTMHSDREDDTASVSTATRVMKAIYSSFKDDVDLKQDLRCDTIDTREEY
EMKDPTNGYYNVRAHEDRPSSRAVLYADYRAPGPARFDGRPSSRLSHSSGYAQLNTYSRGPASDYGPE
PTPPGPAAPAGTDTTSQLSYENYEKFNSHPFPGAAGYPTYRLGYPQAPPSGLERTPYEAYDPIGKYAT
ATRFSYTSQHSDYGQRFQQRMQTHV

| | | |
|---|---|---|
| NOV12p, CG51373-13 | SEQ ID NO: 349 | 2290 bp |
| DNA Sequence | ORF Start: ATG at 11 | ORF Stop: at 2282 |

CACCAAGCTTATGCTGAGCCTCCTCGTCTGGATCCTCACTCTCTCCGATACTTTCTCCCAAGGGACCC
AGACCCGCTTCAGCCAGGAGCCAGCTGACCAGACGGTGGTGGCTGGACAGCGGGCCGTGCTCCCCTGT
GTGCTGCTCAACTACTCTGGAATTGTGCAATGGACCAAGGACGGGCTGGCCCTGGGCATGGGCCAGGG
CCTCAAAGCCTGGCCACGGTACCGGGTTGTGGGCTCCGCAGACGCTGGGCAGTACAACCTGGAGATCA
CAGATGCTGAGCTCTCTGACGACGCCTCTTACGAGTGCCAGGCCACGGAGGCCGCCCTGCGCTCTCGG
CGGGCCAAACTCACCGTGCTCATCCCCCCAGAGGACACCAGGATTGACGGAGGCCCTGTGATTCTACT
GCAGGCAGGCACCCCCCACAACCTCACATGCCGGGCCTTCAATGCGAAGCCTGCTGCCACCATCATCT
GGTTCCGGGACGGGACGCAGCAGGAGGGCGCTGTGGCCAGCACGGAATTGCTGAAGGATGGGAAGAGG
GAGACCACCGTGAGCCAACTGCTTATTAACCCCACGGACCTGGACATAGGGCGTGTCTTCACTTGCCG
AAGCATGAACGAAGCCATCCCTAGTGGCAAGGAGACTTCCATCGAGCTGGATGTGCACCACCCTCCTA
ACAGCCAACCCGGAGATCTTGGGCTACAGGTGGGCCAAAGGGGGTTTCTTGATTGAAGACGCCCACGA
GAGTCGCTATGAGACAAATGTGGATTATTCCTTTTTCACGGAGCCTGTGTCTTGTGAGGTCCACAACA
AAGTGGGAAGCACCAATGTCAGCACTTTAGTAAATGTCCACTTTGCTCCCCGGATTGTAGTTGACCCC
AAACCCACAACCACAGACATTGGCTCTGATGTGACCCTTACCTGTGTCTGGGTTGGGAATCCCCCCCT
CACTCTCACCTGGACCAAAAAGGACTCAAATATGGTCCTGAGTAACAGCAACCAGCTGCTGCTGAAGT
CGGTGACTCAGGCAGACGCTGGCACCTACACCTGCCGGGCCATCGTGCCTCGAATCGGAGTGGCTGAG
CGGGAGGTGCCGCTCTATGTGAACGGGCCCCCCATCATCTCCAGTGAGGCAGTGCAGTATGCTGTGAG
GGGTGACGGTGGCAAGGTGGAGTGTTTCATTGGGAGCACACCACCCCCAGACCGCATAGCATGGGCCT
GGAAGGAGAACTTCTTGGAGGTGGGGACCCTGGAACGCTATACAGTGGAGAGGACCAACTCAGGCAGT
GGGGTGCTATCCACGCTCACCATCAACAATGTCATGGAGGCCGACTTTCAGACTCACTACAACTGCAC
CGCCTGGAACAGCTTCGGGCCAGGCACAGCCATCATCCAGCTGGAAGAGCGAGAGGTGTTACCTGTGG
GCATCATAGCTGGGGCCACCATCGGCGCGAGCATCCTGCTCATCTTCTTCTTCATCGCCTTGGTATTC
TTCCTCTACCGGCGCCGCAAAGGCAGTCGCAAAGACGTGACCCTGAGGAAGCTGGATATCAAGGTGGA
GACAGTGAACCGAGAGCCACTTACGATGCATTCTGACCGGGAGGATGACACCGCCAGCGTCTCCACAG
CAACCCGGGTCATGAAGGCCATCTACTCGTCGTTTAAGGATGATGTGGATCTGAAGCAGGACCTGCGC
TGCGACACCATCGACACCCGGGAGGAGTATGAGATGAAGGACCCCACCAATGGCTACTACAACGTGCG
TGCCCATGAAGACCGCCCGTCTTCCAGGGCAGTGCTCTATGCTGACTACCGTGCCCCTGGCCCTGCCC
GCTTCGACGGCCGCCCCTCATCCCGTCTCTCCCACTCCAGCGGCTATGCCCAGCTCAACACCTATAGC
CGGGGCCCTGCCTCTGACTATGGCCCTGAGCCCACACCCCCTGGCCCTGCTGCCCCAGCTGGCACTGA
CACAACCAGCCAGCTGTCCTACGAGAACTATGAGAAGTTCAACTCCCATCCCTTCCCTGGGGCAGCTG
GGTACCCCACCTACCGACTGGGCTACCCCCAGGCCCCACCCTCTGGCCTGGAGCGGACCCCATATGAG
GCGTATGACCCCATTGGCAAGTACGCCACAGCCACTCGATTCTCCTACACCTCCCAGCACTCGGACTA
CGGCCAGCGATTCCAGCAGCGCATGCAGACTCACGTGGTCGACGGC

| | | | |
|---|---|---|---|
| NOV12p, CG51373-13 | SEQ ID NO: 350 | 757 aa | MW at 83534.8kD |
| Protein Sequence | | | |

MLSLLVWILTLSDTFSQGTQTRFSQEPADQTVVAGQRAVLPCVLLNYSGIVQWTKDGLALGMGQGLKA
WPRYRVVGSADAGQYNLEITDAELSDDASYECQATEAALRSRRAKLTVLIPPEDTRIDGGPVILLQAG
TPHNLTCRAFNAXPAATIIWFRDGTQQEGAVASTELLKDGKRETTVSQLLINPTDLDIGRVFTCRSMN
EAIPSGKETSIELDVHHPPTVTLSIEPQTVQEGERVVFTCQATANPEILGYRWAKGGFLIEDAHESRY
ETNVDYSFFTEPVSCEVHNKVGSTNVSTLVNVHFAPRIVVDPKPTTTDIGSDVTLTCVWVGNPPLTLT
WTKKDSNMVLSNSNQLLLKSVTQADAGTYTCRAIVPRIGVAEREVPLYVNGPPIISSEAVQYAVRGDG
GKVECFIGSTPPPDRIAWAWKENFLEVGTLERYTVERTNSGSGVLSTLTINNVMEADFQTHYNCTAWN
SFGPGTAIIQLEEREVLPVGIIAGATIGASILLIFFFIALVFFLYRRRKGSRKDVTLRKLDIKVETVN
REPLTMHSDREDDTASVSTATRVMKAIYSSFKDDVDLKQDLRCDTIDTREEYEMKDPTNGYYNVRAHE
DRPSSRAVLYADYRAPGPARFDGRPSSRLSHSSGYAQLNTYSRGPASDYGPEPTPPGPAAPAGTDTTS
QLSYENYEKFNSHPFPGAAGYPTYRLGYPQAPPSGLERTPYEAYDPIGKYATATRFSYTSQHSDYGQR
FQQRMQTHV

| | | |
|---|---|---|
| NOV12q, CG51373-14 | SEQ ID NO: 351 | 2290 bp |
| DNA Sequence | ORF Start: at 11 | ORF Stop: at 2282 |

CACCAAGCTTCAAGGGACCCAGACCCGCTTCAGCCAGGAGCCAGCTGACCAGACGGTGGTGGCTGGAC
AGCGGGCCGTGCTCCCCTGTGTGCTGCTCAACTACTCTGGAATTGTGCAATGGACCAAGGACGGGCTG
GCCCTGGGCATGGGCCAGGGCCTCAAAGCCTGGCCACGGTACCGGGTTGTGGGCTCCGCAGACGCTGG
GCAGTACAACCTGGAGATCACAGATGCTGAGCTCTCTGACGACGCCTCTTACGAGTGCCAGGCCACGG
AGGCCGCCCTGCGCTCTCGGCGGGCCAAACTCACCGTGCTCATCCCCCCAGAGGACACCAGGATTGAC
GGAGGCCCTGTGATTCTACTGCAGGCAGGCACCCCCCACAACCTCACATGCCGGGCCTTCAATGCGAA
GCCTGCTGCCACCATCATCTGGTTCCGGGACGGGACGCAGCAGGAGGGCGCTGTGGCCAGCACGGAAT
TGCTGAAGGATGGGAAGAGGGAGACCACCGTGAGCCAACTGCTTATTAACCCCACGGACCTGGACATA
GGGCGTGTCTTCACTTGCCGAAGCATGAACGAAGCCATCCCTAGTGGCAAGGAGACTTCCATCGAGCT
GGATGTGCACCACCCTCCTACAGTGACCCTGTCCATTGAGCCACAGACGGTGCAGGAGGGTGAGCGTG
TTGTCTTTACCTGCCAGGCCACAGCCAACCCGGAGATCTTGGGCTACAGGTGGGCCAAAGGGGGTTTC
TTGATTGAAGACGCCCACGAGAGTCGCTATGAGACAAATGTGGATTATTCCTTTTTCACGGAGCCTGT
GTCTTGTGAGGTTCACAACAAAGTGGGAAGCACCAATGTCAGCACTTTAGTAAATGTCCACTTTGCTC
CCCGGATTGTAGTTGACCCCAAACCCACAACCACAGACATTGGCTCTGATGTGACCCTTACCTGTGTC
TGGGTTGGGAATCCCCCCCTCACTCTCACCTGGACCAAAAAGGACTCAAATATGGGGCCCAGGCCTCC
TGGCTCCCCACCCGAGGCTGCTCTCTCTGCCCAGGTCCTGAGTAACAGCAACCAGCTGCTGCTGAAGT
CGGTGACTCAGGCAGACGCTGGCACCTACACCTGCCGGGCCATCGTGCCTCGAATCGGAGTGGCTGAG
CGGGAGGTGCCGCTCTATGTGAACGGGCCCCCCATCATCTCCAGTGAGGCAGTGCAGTATGCTGTGAG
GGGTGACGGTGGCAAGGTGGAGTGTTTCATTGGGAGCACACCACCCCCAGACCGCATAGCATGGGCCT
GGAAGGAGAACTTCTTGGAGGTGGGGACCCTGGAACGCTATACAGTGGAGAGGACCAACTCAGGCAGT
GGGGTGCTATCCACGCTCACCATCAACAATGTCATGGAGGCCGACTTTCAGACTCACTACAACTGCAC
CGCCTGGAACAGCTTCGGGCCAGGCACAGCCATCATCCAGCTGGAAGAGCGAGAGGTGTTACCTGTGG
GCATCATAGCTGGGGCCACCATCGGCGCGAGCATCCTGCTCATCTTCTTCTTCATCGCCTTGGTATTC
TTCCTCTACCGGCGCCGCAAAGGCAGTCGCAAAGACGTGACCCTGAGGAAGCTGGATATCAAGGTGGA
GACAGTGAACCGAGAGCCACTTACGATGCATTCTGACCGGGAGGATGACACCGCCAGCGTCTCCACAG
CAACCCGGGTCATGAAGGCCATCTACTCGTCGTTTAAGGATGATGTGGATCTGAAGCAGGACCTGCGC
TGCGACACCATCGACACCCGGGAGGAGTATGAGATGAAGGACCCCACCAATGGCTACTACAACGTGCG
TGCCCATGAAGACCGCCCGTCTTCCAGGGCAGTGCTCTATGCTGACTACCGTGCCCCTGGCCCTGCCC
GCTTCGACGGCCGCCCCTCATCCCGTCTCTCCCACTCCAGCGGCTATGCCCAGCTCAACACCTATAGC
CGGGGCCCTGCCTCTGACTATGGCCCTGAGCCCACACCCCCTGGCCCTGCTGCCCCAGCTGGCACTGA
CACAACCAGCCAGCTGTCCTACGAGAACTATGAGAAGTTCAACTCCCATCCCTTCCCTGGGGCAGCTG
GGTACCCCACCTACCGACTGGGCTACCCCCAGGCCCCACCCTCTGGCCTGGAGCGGACCCCATATGAG
GCGTATGACCCCATTGGCAAGTACGCCACAGCCACTCGATTCTCCTACACCTCCCAGCACTCGGACTA
CGGCCAGCGATTCCAGCAGCGCATGCAGACTCACGTGGTCGACGGC

| | | | |
|---|---|---|---|
| NOV12q, CG51373-14 | SEQ ID NO: 352 | 757 aa | MW at 83227.3kD |
| Protein Sequence | | | |

QGTQTRFSQEPADQTVVAGQRAVLPCVLLNYSGIVQWTKDGLALGMGQGLKAWPRYRVVGSADAGQYN
LEITDAELSDDASYECQATEAALRSRPAKLTVLIPPEDTRIDGGPVILLQAGTPHNLTCRAFNAKPAA
TIIWFRDGTQQEGAVASTELLKDGKRETTVSQLLINPTDLDIGRVFTCRSMNEAIPSGKETSIELDVH
HPPTVTLSIEPQTVQEGERVVFTCQATANPEILGYRWAKGGFLIEDAHESRYETNVDYSFFTEPVSCE
VHNKVGSTNVSTLVNVHFAPRIVVDPKPTTTDIGSDVTLTCVWVGNPPLTLTWTKKDSNMGPRPPGSP
PEAALSAQVLSNSNQLLLKSVTQADAGTYTCRAIVPRIGVAEREVPLYVNGPPIISSEAVQYAVRGDG
GKVECFIGSTPPPDRIAWAWKENFLEVGTLERYTVERTNSGSGVLSTLTINNVMEADFQTHYNCTAWN
SFGPGTAIIQLEEREVLPVGIIAGATIGASILLIFFFIALVFFLYRRRKGSRKDVTLRKLDIKVETVN
REPLTMHSDREDDTASVSTATRVMKAIYSSFKDDVDLKQDLRCDTIDTREEYEMKDPTNGYYNVRAHE
DRPSSRAVLYADYRAPGPARFDGRPSSRLSHSSGYAQLNTYSRGPASDYGPEPTPPGPAAPAGTDTTS
QLSYENYEKFNSHPFPGAAGYPTYRLGYPQAPPSGLERTPYEAYDPIGKYATATRFSYTSQHSDYGQR
FQQRMQTHV

| | | |
|---|---|---|
| NOV12r, SNP13377352 of | SEQ ID NO: 353 | 2128 bp |
| CG51373-08, DNA Sequence | ORF Start: ATG at 31 | ORF Stop: TAG at 2119 |
| | SNP Pos: 487 | SNP Change: T to C |

CAATGGACCAAGGACGGGCTGGCCCTGGGCATGGGCCAGGGCCTCAAAGCCTGGCCACGGTACCGGGT
TGTGGGCTCCGCAGACGCTGGGCAGTACAACCTGGAGATCACAGATGCTGAGCTCTCTGACGACGCCT
CTTACGAGTGCCAGGCCACGGAGGCCGCCCTGCGCTCTCGGCGGGCCAAACTCACCGTGCTCATCCCC
CCAGAGGACACCAGGATTGACGGAGGCCCTGTGATTCTACTGCAGGCAGGCACCCCCCACAACCTCAC
ATGCCGGGCCTTCAATGCGAAGCCTGCTGCCACCATCATCTGGTTCCGGGACGGGACGCAGCAGGAGG
GCGCTGTGGCCAGCACGGAATTGCTGAAGGATGGGAAGAGGGAGACCACCGTGAGCCAACTGCTTATT
AACCCCACGGACCTGGACATAGGGCGTGTCTTCACTTGCCGAAGCATGAACGAAGCCATCCCTAGTGG
CAAGGAGACTCCCATCGAGCTGGATGTGCACCACCCTCCTACAGTGACCCTGTCCATTGAGCCACAGA
CGGTGCAGGAGGGTGAGCGTGTTGTCTTTACCTGCCAGGCCACAGCCAACCCCGAGATCTTGGACTAC
AGGTGGGCCAAAGGGGGTTTCTTGATTGAAGACGCCCACGAGAGTCGCTATGAGACAAATGTGGATTA
TTCCTTTTTCACGGAGCCTGTGTCTTGTGAGGTTCACAACAAAGTGGGAAGCACCAATGTCAGCACTT
GATGTGACCCTTACCTGTGTCTGGGTTGGGAATCCCCCCCTCACTCTCACCTGGACCAAAAAGGACTC
AAATATGGTCCTGAGTAACAGCAACCAGCTGCTGCTGAAGTCGGTGACTCAGGCAGACGCTGGCACCT
ACACCTGCCGGGCCATCGTGCCTCGAATCGGAGTGGCTGAGCGGGAGGTGCCGCTCTATGTGAACGGG
CCCCCCATCATCTCCAGTGAGGCAGTGCAGTATGCTGTGAGGGGTGACGGTGGCAAGGTGGAGTGTTT
CATTGGGAGCACACCACCCCCAGACCGCATAGCATGGGCCTGGAAGGAGAACTTCTTGGAGGTGGGGA
CCCTGGAACGCTATACAGTGGAGAGGACCAACTCAGGCAGTGGGGTGCTATCCACGCTCACCATCAAC
AATGTCATGGAGGCCGACTTTCAGACTCACTACAACTGCACCGCCTGGAACAGCTTCGGGCCAGGCAC
AGCCATCATCCAGCTGGAAGAGCGAGAGGTGTTACCTGTGGGCATCATAGCTGGGGCCACCATCGGCG
CGAGCATCCTGCTCATCTTCTTCTTCATCGCCTTGGTATTCTTCCTCTACCGGCGCCGCAAAGGCAGT
CGCAAAGACGTGACCCTGAGGAAGCTGGATATCAAGGTGGAGACAGTGAACCGAGAGCCACTTACGAT
GCATTCTGACCGGGAGGATGACACCGCCAGCGTCTCCACAGCAACCCGGGTCATGAAGGCCATCTACT
CGTCGTTTATGGATGATGTGGATCTGAAGCAGGACCTGCGCTGCGACACCATCGACACCCGGGAGGAG
TATGAGATGAAGGACCCCACCAATGGCTACTACAACGTGCGTGCCCATGAAGACCGCCCGTCTTCCAG
GGCAGTGCTCTATGCTGACTACCGTGCCCCTGGCCCTGCCCGCTTCGACGGCCGCCCCTCATCCCGTC
TCTCCCACTCCAGCGGCTATGCCCAGCTCAACACCTATAGCCGGGGCCCTGCCTCTGACTATGGCCCT
GAGCCCACACCCCCTGGCCCTGCTGCCCCAGCTGGCACTGACACAACCAGCCAGCTGTCCTACGAGAA
CTATGAGAAGTTCAACTCCCATCCCTTCCCTGGGGCAGCTGGGTACCCCACCTACCGACTGGGCTACC
CCCAGGCCCCACCCTCTGGCCTGGAGCGGACCCCATATGAGGCGTATGACCCCATTGGCAAGTACGCC
ACAGCCACTCGATTCTCCTACACCTCCCAGCACTCGGACTACGGCCAGCGATTCCAGCAGCGCATGCA
GACTCACGTGTAGGGGCCAG

| | | | |
|---|---|---|---|
| NOV12r, SNP13377352 of | SEQ ID NO: 354 | 696 aa | MW at 76985.3kD |
| CG51373-08, Protein | SNP Pos: 153 | | SNP Change: Ser to Pro |
| Sequence | | | |

MGQGLKAWPRYRVVGSADAGQYNLEITDAELSDDASYECQATEAALRSRRAKLTVLIPPEDTRIDGGP
VILLQAGTPHNLTCRAFNAKPAATIIWFRDGTQQEGAVASTELLKDGKRETTVSQLLINPTDLDIGRV
FTCRSMNEAIPSGKETPIELDVHHPPTVTLSIEPQTVQEGERVVFTCQATANPEILDYRWAKGGFLIE
DAHESRYETNVDYSFFTEPVSCEVHNKVGSTNVSTLVNVHFAPRIVVDPKPTTTDIGSDVTLTCVWVG
NPPLTLTWTKKDSNMVLSNSNQLLLKSVTQADAGTYTCRAIVPRIGVAEREVPLYVNGPPIISSEAVQ
YAVRGDGGKVECFIGSTPPPDRIAWAWKENFLEVGTLERYTVERTNSGSGVLSTLTINNVMEADFQTH
YNCTAWNSFGPGTAIIQLEEREVLPVGIIAGATIGASILLIFFFIALVFFLYRRRKGSRKDVTLRKLD
IKVETVNREPLTMHSDREDDTASVSTATRVMKAIYSSFMDDVDLKQDLRCDTIDTREEYEMKDPTNGY
YNVRAHEDRPSSRAVLYADYEAPGPARFDGRPSSRLSHSSGYAQLNTYSRGPASDYGPEPTPPGPAAP
AGTDTTSQLSYENYEKFNSHPFPGAAGYPTYRLGYPQAPPSGLERTPYEAYDPIGKYATATRFSYTSQ
HSDYGQRFQQRMQTHV

| | | |
|---|---|---|
| NOV12s, SNP13379746 of | SEQ ID NO: 355 | 2128 bp |
| CG51373-08, DNA Sequence | ORF Start: ATG at 31 | ORF Stop: TAG at 2119 |
| | SNP Pos: 597 | SNP Change: C to G |

CAATGGACCAAGGACGGGCTGGCCCTGGGCATGGGCCAGGGCCTCAAAGCCTGGCCACGGTACCGGGT
TGTGGGCTCCGCAGACGCTGGGCAGTACAACCTGGAGATCACAGATGCTGAGCTCTCTGACGACGCCT
CTTACGAGTGCCAGGCCACGGAGGCCGCCCTGCGCTCTCGGCGGGCCAAACTCACCGTGCTCATCCCC
CCAGAGGACACCAGGATTGACGGAGGCCCTGTGATTCTACTGCAGGCAGGCACCCCCCACAACCTCAC
ATGCCGGGCCTTCAATGCGAAGCCTGCTGCCACCATCATCTGGTTCCGGGACGGGACGCAGCAGGAGG
GCGCTGTGGCCAGCACGGAATTGCTGAAGGATGGGAAGAGGGAGACCACCGTGAGCCAACTGCTTATT
AACCCCACGGACCTGGACATAGGGCGTGTCTTCACTTGCCGAAGCATGAACGAAGCCATCCCTAGTGG
CAAGGAGACTTCCATCGAGCTGGATGTGCACCACCCTCCTACAGTGACCCTGTCCATTGAGCCACAGA
CGGTGCAGGAGGGTGAGCGTGTTGTCTTTACCTGCCAGGCCACAGCCAACCCGGAGATCTTGGACTAC
AGGTGGGCCAAAGGGGGTTTCTTGATTGAAGACGCCCACGAGAGTCGCTATGAGACAAATGTGGATTA
TTCCTTTTTCACGGAGCCTGTGTCTTGTGAGGTTCACAACAAAGTGGGAAGCACCAATGTCAGCACTT
TAGTAAATGTCCACTTTGCTCCCCGGATTGTAGTTGACCCCAAACCCACAACCACAGACATTGGCTCT
GATGTGACCCTTACCTGTGTCTGGGTTGGGAATCCCCCCCTCACTCTCACCTGGACCAAAAAGGACTC
AAATATGGTCCTGAGTAACAGCAACCAGCTGCTGCTGAAGTCGGTGACTCAGGCAGACGCTGGCACCT
ACACCTGCCGGGCCATCGTGCCTCGAATCGGAGTGGCTGAGCGGGAGGTGCCGCTCTATGTGAACGGG
CCCCCCATCATCTCCAGTGAGGCAGTGCAGTATGCTGTGAGGGGTGACGGTGGCAAGGTGGAGTGTTT
CATTGGGAGCACACCACCCCCAGACCGCATAGCATGGGCCTGGAAGGAGAACTTCTTGGAGGTGGGGA
CCCTGGAACGCTATACAGTGGAGAGGACCAACTCAGGCAGTGGGGTGCTATCCACGCTCACCATCAAC
AATGTCATGGAGGCCGACTTTCAGACTCACTACAACTGCACCGCCTGGAACAGCTTCGGGCCAGGCAC
AGCCATCATCCAGCTGGAAGAGCGAGAGGTGTTACCTGTGGGCATCATAGCTGGGGCCACCATCGGCG
CGAGCATCCTGCTCATCTTCTTCTTCATCGCCTTGGTATTCTTCCTCTACCGGCGCCGCAAAGGCAGT
CGCAAAGACGTGACCCTGAGGAAGCTGGATATCAAGGTGGAGACAGTGAACCGAGAGCCACTTACGAT
GCATTCTGACCGGGAGGATGACACCGCCAGCGTCTCCACAGCAACCCGGGTCATGAAGGCCATCTACT
CGTCGTTTATGGATGATGTGGATCTGAAGCAGGACCTGCGCTGCGACACCATCGACACCCGGGAGGAG
TATGAGATGAAGGACCCCACCAATGGCTACTACAACGTGCGTGCCCATGAAGACCGCCCGTCTTCCAG
GGCAGTGCTCTATGCTGACTACCGTGCCCCTGGCCCTGCCCGCTTCGACGGCCGCCCCTCATCCCGTC
TCTCCCACTCCAGCGGCTATGCCCAGCTCAACACCTATAGCCGGGGCCCTGCCTCTGACTATGGCCCT
GAGCCCACACCCCCTGGCCCTGCTGCCCCAGCTGGCACTGACACAACCAGCCAGCTGTCCTACGAGAA
CTATGAGAAGTTCAACTCCCATCCCTTCCCTGGGGCAGCTGGGTACCCCACCTACCGACTGGGCTACC
CCCAGGCCCCACCCTCTGGCCTGGAGCGGACCCCATATGAGGCGTATGACCCCATTGGCAAGTACGCC
ACAGCCACTCGATTCTCCTACACCTCCCAGCACTCGGACTACGGCCAGCGATTCCAGCAGCGCATGCA
GACTCACGTGTAGGGGCCAG

| | | | |
|---|---|---|---|
| NOV12s, SNP13379746 of | SEQ ID NO: 356 | 696 aa | MW at 76975.3kD |
| CG51373-08, Protein | SNP Pos: 189 | | SNP Change: Pro to Pro |
| Sequence | | | |

MGQGLKAWPRYRVVGSADAGQYNLEITDAELSDDASYECQATEAALRSRRAKLTVLIPPEDTRIDGGP
VILLQAGTPHNLTCRAFNAKPAATIIWFRDGTQQEGAVASTELLKDGKRETTVSQLLINPTDLDIGRV
FTCRSMNEAIPSGKETSIELDVHHPPTVTLSIEPQTVQEGERVVFTCQATANPEILDYRWAKGGFLIE
DAHESRYETNVDYSFFTEPVSCEVHNKVGSTNVSTLVNVHFAPRIVVDPKPTTTDIGSDVTLTCVWVG
NPPLTLTWTKKDSNMVLSNSNQLLLKSVTQADAGTYTCRAIVPRIGVAEREVPLYVNGPPIISSEAVQ
YAVRGDGGKVECFIGSTPPPDRIAWAWKENFLEVGTLERYTVERTNSGSGVLSTLTINNVMEADFQTH
YNCTAWNSFGPGTAIIQLEEREVLPVGIIAGATIGASILLIFFFIALVFFLYRRRKGSRKDVTLRKLD
IKVETVNREPLTMHSDREDDTASVSTATRVMKAIYSSFMDDVDLKQDLRCDTIDTREEYEMKDPTNGY
YNVRAHEDRPSSRAVLYADYEAPGPARFDGRPSSRLSHSSGYAQLNTYSRGPASDYGPEPTPPGPAAP
AGTDTTSQLSYENYEKFNSHPFPGAAGYPTYRLGYPQAPPSGLERTPYEAYDPIGKYATATRFSYTSQ
HSDYGQRFQQRMQTHV

| | | |
|---|---|---|
| NOV12t, SNP13374859 of | SEQ ID NO: 357 | 2128 bp |
| CG51373-08, DNA Sequence | ORF Start: ATG at 31 | ORF Stop: TAG at 2119 |
| | SNP Pos: 961 | SNP Change: C to T |

CAATGGACCAAGGACGGGCTGGCCCTGGGCATGGGCCAGGGCCTCAAAGCCTGGCCACGGTACCGGGT
TGTGGGCTCCGCAGACGCTGGGCAGTACAACCTGGAGATCACAGATGCTGAGCTCTCTGACGACGCCT
CTTACGAGTGCCAGGCCACGGAGGCCGCCCTGCGCTCTCGGCGGGCCAAACTCACCGTGCTCATCCCC
CCAGAGGACACCAGGATTGACGGAGGCCCTGTGATTCTACTGCAGGCAGGCACCCCCCACAACCTCAC
ATGCCGGGCCTTCAATGCGAAGCCTGCTGCCACCATCATCTGGTTCCGGGACGGGACGCAGCAGGAGG
GCGCTGTGGCCAGCACGGAATTGCTGAAGGATGGGAAGAGGGAGACCACCGTGAGCCAACTGCTTATT
AACCCCACGGACCTGGACATAGGGCGTGTCTTCACTTGCCGAAGCATGAACGAAGCCATCCCTAGTGG
CAAGGAGACTTCCATCGAGCTGGATGTGCACCACCCTCCTACAGTGACCCTGTCCATTGAGCCACAGA
CGGTGCAGGAGGGTGAGCGTGTTGTCTTTACCTGCCAGGCCACAGCCAACCCCGAGATCTTGGACTAC
AGGTGGGCCAAAGGGGGTTTCTTGATTGAAGACGCCCACGAGAGTCGCTATGAGACAAATGTGGATTA
TTCCTTTTTCACGGAGCCTGTGTCTTGTGAGGTTCACAACAAAGTGGGAAGCACCAATGTCAGCACTT
TAGTAAATGTCCACTTTGCTCCCCGGATTGTAGTTGACCCCAAACCCACAACCACAGACATTGGCTCT
GATGTGACCCTTACCTGTGTCTGGGTTGGGAATCCCCCCCTCACTCTCACCTGGACCAAAAAGGACTC
AAATATGGTCCTGAGTAACAGCAACCAGCTGCTGCTGAAGTCGGTGACTCAGGCAGACGCTGGCACCT
ACACCTGCTGGGCCATCGTGCCTCGAATCGGAGTGGCTGAGCGGGAGGTGCCGCTCTATGTGAACGGG
CCCCCCATCATCTCCAGTGAGGCAGTGCAGTATGCTGTGAGGGGTGACGGTGGCAAGGTGGAGTGTTT
CATTGGGAGCACACCACCCCCAGACCGCATAGCATGGGCCTGGAAGGAGAACTTCTTGGAGGTGGGGA
CCCTGGAACGCTATACAGTGGAGAGGACCAACTCAGGCAGTGGGGTGCTATCCACGCTCACCATCAAC
AATGTCATGGAGGCCGACTTTCAGACTCACTACAACTGCACCGCCTGGAACAGCTTCGGGCCAGGCAC
AGCCATCATCCAGCTGGAAGAGCGAGAGGTGTTACCTGTGGGCATCATAGCTGGGGCCACCATCGGCG
CGAGCATCCTGCTCATCTTCTTCTTCATCGCCTTGGTATTCTTCCTCTACCGGCGCCGCAAAGGCAGT
CGCAAAGACGTGACCCTGAGGAAGCTGGATATCAAGGTGGAGACAGTGAACCGAGAGCCACTTACGAT
GCATTCTGACCGGGAGGATGACACCGCCAGCGTCTCCACAGCAACCCGGGTCATGAAGGCCATCTACT
CGTCGTTTATGGATGATGTGGATCTGAAGCAGGACCTGCGCTGCGACACCATCGACACCCGGGAGGAG
TATGAGATGAAGGACCCCACCAATGGCTACTACAACGTGCGTGCCCATGAAGACCGCCCGTCTTCCAG
GGCAGTGCTCTATGCTGACTACCGTGCCCCTGGCCCTGCCCGCTTCGACGGCCGCCCCTCATCCCGTC
TCTCCCACTCCAGCGGCTATGCCCAGCTCAACACCTATAGCCGGGGCCCTGCCTCTGACTATGGCCCT
GAGCCCACACCCCCTGGCCCTGCTGCCCCAGCTGGCACTGACACAACCAGCCAGCTGTCCTACGAGAA
CTATGAGAAGTTCAACTCCCATCCCTTCCCTGGGGCAGCTGGGTACCCCACCTACCGACTGGGCTACC
CCCAGGCCCCACCCTCTGGCCTGGAGCGGACCCCATATGAGGCGTATGACCCCATTGGCAAGTACGCC
ACAGCCACTCGATTCTCCTACACCTCCCAGCACTCGGACTACGGCCAGCGATTCCAGCAGCGCATGCA
GACTCACGTGTAGGGGCCAG

| | | | |
|---|---|---|---|
| NOV12t, SNP13374859 of | SEQ ID NO: 358 | 696 aa | MW at 77005.3kD |
| CG51373-08, Protein | SNP Pos: 311 | | SNP Change: Arg to Trp |
| Sequence | | | |

MGQGLKAWPRYRVVGSADAGQYNLEITDAELSDDASYECQATEAALRSRRAKLTVLIPPEDTRIDGGP
VILLQAGTPHNLTCRAFNAKPAATIIWFRDGTQQEGAVASTELLKDGKRETTVSQLLINPTDLDIGRV
FTCRSMNEAIPSGKETSIELDVHHPPTVTLSIEPQTVQEGERVVFTCQATANPEILDYRWAKGGFLIE
DAHESRYETNVDYSFFTEPVSCEVHNKVGSTNVSTLVNVHFAPRIVVDPKPTTTDIGSDVTLTCVWVG
NPPLTLTWTKKDSNMVLSNSNQLLLKSVTQADAGTYTCWAIVPRIGVAEREVPLYVNGPPIISSEAVQ
YAVRGDGGKVECFIGSTPPPDRIAWAWKENFLEVGTLERYTVERTNSGSGVLSTLTINNVMEADFQTH
YNCTAWNSFGPGTAIIQLEEREVLPVGIIAGATIGASILLIFFFIALVFFLYRRRKGSRKDVTLRKLD
IKVETVNREPLTMHSDREDDTASVSTATRVMKAIYSSFMDDVDLKQDLRCDTIDTREEYEMKDPTNGY
YNVRAHEDRPSSRAVLYADYRAPGPARFDGRPSSRLSHSSGYAQLNTYSRGPASDYGPEPTPPGPAAP
AGTDTTSQLSYENYEKFNSHPFPGAAGYPTYRLGYPQAPPSGLERTPYEAYDPIGKYATATRFSYTSQ
HSDYGQRFQQRMQTHV

| | | |
|---|---|---|
| NOV12u, SNP13374858 of | SEQ ID NO: 359 | 2128 bp |
| CG51373-08, DNA Sequence | ORF Start: ATG at 31 | ORF Stop: TAG at 2119 |
| | SNP Pos: 1340 | SNP Change: T to C |

CAATGGACCAAGGACGGGCTGGCCCTGGGCATGGGCCAGGGCCTCAAAGCCTGGCCACGGTACCGGGT
TGTGGGCTCCGCAGACGCTGGGCAGTACAACCTGGAGATCACAGATGCTGAGCTCTCTGACGACGCCT
CTTACGAGTGCCAGGCCACGGAGGCCGCCCTGCGCTCTCGGCGGGCCAAACTCACCGTGCTCATCCCC
CCAGAGGACACCAGGATTGACGGAGGCCCTGTGATTCTACTGCAGGCAGGCACCCCCCACAACCTCAC
ATGCCGGGCCTTCAATGCGAAGCCTGCTGCCACCATCATCTGGTTCCGGGACGGGACGCAGCAGGAGG
GCGCTGTGGCCAGCACGGAATTGCTGAAGGATGGGAAGAGGGAGACCACCGTGAGCCAACTGCTTATT
AACCCCACGGACCTGGACATAGGGCGTGTCTTCACTTGCCGAAGCATGAACGAAGCCATCCCTAGTGG
CAAGGAGACTTCCATCGAGCTGGATGTGCACCACCCTCCTACAGTGACCCTGTCCATTGAGCCACAGA
CGGTGCAGGAGGGTGAGCGTGTTGTCTTTACCTGCCAGGCCACAGCCAACCCCGAGATCTTGGACTAC
AGGTGGGCCAAAGGGGGTTTCTTGATTGAAGACGCCCACGAGAGTCGCTATGAGACAAATGTGGATTA
TTCCTTTTTCACGGAGCCTGTGTCTTGTGAGGTTCACAACAAAGTGGGAAGCACCAATGTCAGCACTT
TAGTAAATGTCCACTTTGCTCCCCGGATTGTAGTTGACCCCAAACCCACAACCACAGACATTGGCTCT
GATGTGACCCTTACCTGTGTCTGGGTTGGGAATCCCCCCCTCACTCTCACCTGGACCAAAAAGGACTC
AAATATGGTCCTGAGTAACAGCAACCAGCTGCTGCTGAAGTCGGTGACTCAGGCAGACGCTGGCACCT
ACACCTGCCGGGCCATCGTGCCTCGAATCGGAGTGGCTGAGCGGGAGGTGCCGCTCTATGTGAACGGG
CCCCCCATCATCTCCAGTGAGGCAGTGCAGTATGCTGTGAGGGGTGACGGTGGCAAGGTGGAGTGTTT
CATTGGGAGCACACCACCCCCAGACCGCATAGCATGGGCCTGGAAGGAGAACTTCTTGGAGGTGGGGA
CCCTGGAACGCTATACAGTGGAGAGGACCAACTCAGGCAGTGGGGTGCTATCCACGCTCACCATCAAC
AATGTCATGGAGGCCGACTTTCAGACTCACTACAACTGCACCGCCTGGAACAGCTTCGGGCCAGGCAC
AGCCATCATCCAGCTGGAAGAGCGAGAGGTGTTACCTGTGGGCATCACAGCTGGGGCCACCATCGGCG
CGAGCATCCTGCTCATCTTCTTCTTCATCGCCTTGGTATTCTTCCTCTACCGGCGCCGCAAAGGCAGT
CGCAAAGACGTGACCCTGAGGAAGCTGGATATCAAGGTGGAGACAGTGAACCGAGAGCCACTTACGAT
GCATTCTGACCGGGAGGATGACACCGCCAGCGTCTCCACAGCAACCCGGGTCATGAAGGCCATCTACT
CGTCGTTTATGGATGATGTGGATCTGAAGCAGGACCTGCGCTGCGACACCATCGACACCCGGGAGGAG
TATGAGATGAAGGACCCCACCAATGGCTACTACAACGTGCGTGCCCATGAAGACCGCCCGTCTTCCAG
GGCAGTGCTCTATGCTGACTACCGTGCCCCTGGCCCTGCCCGCTTCGACGGCCGCCCCTCATCCCGTC
TCTCCCACTCCAGCGGCTATGCCCAGCTCAACACCTATAGCCGGGGCCCTGCCTCTGACTATGGCCCT
GAGCCCACACCCCCTGGCCCTGCTGCCCCAGCTGGCACTGACACAACCAGCCAGCTGTCCTACGAGAA
CTATGAGAAGTTCAACTCCCATCCCTTCCCTGGGGCAGCTGGGTACCCCACCTACCGACTGGGCTACC
CCCAGGCCCCACCCTCTGGCCTGGAGCGGACCCCATATGAGGCGTATGACCCCATTGGCAAGTACGCC
ACAGCCACTCGATTCTCCTACACCTCCCAGCACTCGGACTACGGCCAGCGATTCCAGCAGCGCATGCA
GACTCACGTGTAGGGGCCAG

| | | | |
|---|---|---|---|
| NOV12u, SNP13374858 of | SEQ ID NO: 360 | 696 aa | MW at 76963.2kD |
| CG51373-08, Protein | SNP Pos: 437 | | SNP Change: Ile to Thr |
| Sequence | | | |

MGQGLKAWPRYRVVGSADAGQYNLEITDAELSDDASYECQATEAALRSRRAKLTVLIPPEDTRIDGGP
VILLQAGTPHNLTCRAFNAKPAATIIWFRDGTQQEGAVASTELLKDGKRETTVSQLLINPTDLDIGRV
FTCRSMNEAIPSGKETSIELDVHHPPTVTLSIEPQTVQEGERVVFTCQATANPEILDYRWAKGGFLIE
DAHESRYETNVDYSFFTEPVSCEVHNKVGSTNVSTLVNVHFAPRIVVDPKPTTTDIGSDVTLTCVWVG
NPPLTLTWTKKDSNNVLSNSNQLLLKSVTQADAGTYTCRAIVPRIGVAEREVPLYVNGPPIISSEAVQ
YAVRGDGGKVECFIGSTPPPDRIAWAWKENFLEVGTLERYTVERTNSGSGVLSTLTINNVMEADFQTH
YNCTAWNSFGPGTAIIQLEEREVLPVGITAGATIGASILLIFFFIALVFFLYRRRKGSRKDVTLRKLD
IKVETVNREPLTMHSDREDDTASVSTATRVMKAIYSSFMDDVDLKQDLRCDTIDTREEYEMKDPTNGY
YNVRAHEDRPSSRAVLYADYRAPGPARFDGRPSSRLSHSSGYAQLNTYSRGPASDYGPEPTPPGPAAP
AGTDTTSQLSYENYEKFNSHPFPGAAGYPTYRLGYPQAPPSGLERTPYEAYDPIGKYATATRFSYTSQ
HSDYGQRFQQRMQTHV

| | | |
|---|---|---|
| NOV12v, SNP13374856 of | SEQ ID NO: 361 | 2128 bp |
| CG51373-08, DNA Sequence | ORF Start: ATG at 31 | ORF Stop: TAG at 2119 |
| | SNP Pos: 1418 | SNP Change: G to A |

CAATGGACCAAGGACGGGCTGGCCCTGGGCATGGGCCAGGGCCTCAAAGCCTGGCCACGGTACCGGGT
TGTGGGCTCCGCAGACGCTGGGCAGTACAACCTGGAGATCACAGATGCTGAGCTCTCTGACGACGCCT
CTTACGAGTGCCAGGCCACGGAGGCCGCCCTGCGCTCTCGGCGGGCCAAACTCACCGTGCTCATCCCC
CCAGAGGACACCAGGATTGACGGAGGCCCTGTGATTCTACTGCAGGCAGGCACCCCCCACAACCTCAC
ATGCCGGGCCTTCAATGCGAAGCCTGCTGCCACCATCATCTGGTTCCGGGACGGGACGCAGCAGGAGG
GCGCTGTGGCCAGCACGGAATTGCTGAAGGATGGGAAGACGGAGACCACCGTGAGCCAACTGCTTATT
AACCCCACGGACCTGGACATAGGGCGTGTCTTCACTTGCCGAAGCATGAACGAAGCCATCCCTAGTGG
CAAGGAGACTTCCATCGAGCTGGATGTGCACCACCCTCCTACAGTGACCCTGTCCATTGAGCCACAGA
CGGTGCAGGAGGGTGAGCGTGTTGTCTTTACCTGCCAGGCCACAGCCAACCCCGAGATCTTGGACTAC
AGGTGGGCCAAAGGGGGTTTCTTGATTGAAGACGCCCACGAGAGTCGCTATGAGACAAATGTGGATTA
TTCCTTTTTCACGGAGCCTGTGTCTTGTGAGGTTCACAACAAAGTGGGAAGCACCAATGTCAGCACTT
TAGTAAATGTCCACTTTGCTCCCCGGATTGTAGTTGACCCCAAACCCACAACCACAGACATTGGCTCT
GATGTGACCCTTACCTGTGTCTGGGTTGGGAATCCCCCCCTCACTCTCACCTGGACCAAAAAGGACTC
AAATATGGTCCTGAGTAACAGCAACCAGCTGCTGCTGAAGTCGGTGACTCAGGCAGACGCTGGCACCT
ACACCTGCCGGGCCATCGTGCCTCGAATCGGAGTGGCTGAGCGGGAGGTGCCGCTCTATGTGAACGGG
CCCCCCATCATCTCCAGTGAGGCAGTGCAGTATGCTGTGAGGGGTGACGGTGGCAAGGTGGAGTGTTT
CATTGGGAGCACACCACCCCCAGACCGCATAGCATGGGCCTGGAAGGAGAACTTCTTGGAGGTGGGGA
CCCTGGAACGCTATACAGTGGAGAGGACCAACTCAGGCAGTGGGGTGCTATCCACGCTCACCATCAAC
AATGTCATGGAGGCCGACTTTCAGACTCACTACAACTGCACCGCCTGGAACAGCTTCGGGCCAGGCAC
AGCCATCATCCAGCTGGAAGAGCGAGAGGTGTTACCTGTGGGCATCATAGCTGGGGCCACCATCGGCG
CGAGCATCCTGCTCATCTTCTTCTTCATCGCCTTGGTATTCTTCCTCTACCGGCGCCACAAAGGCAGT
CGCAAAGACGTGACCCTGAGGAAGCTGGATATCAAGGTGGAGACAGTGAACCGAGAGCCACTTACGAT
GCATTCTGACCGGGAGGATGACACCGCCAGCGTCTCCACAGCAACCCGGGTCATGAAGGCCATCTACT
CGTCGTTTATGGATGATGTGGATCTGAAGCAGGACCTGCGCTGCGACACCATCGACACCCGGGAGGAG
TATGAGATGAAGGACCCCACCAATGGCTACTACAACGTGCGTGCCCATGAAGACCGCCCGTCTTCCAG
GGCAGTGCTCTATGCTGACTACCGTGCCCCTGGCCCTGCCCGCTTCGACGGCCGCCCCTCATCCCGTC
TCTCCCACTCCAGCGGCTATGCCCAGCTCAACACCTATAGCCGGGGCCCTGCCTCTGACTATGGCCCT
GAGCCCACACCCCCTGGCCCTGCTGCCCCAGCTGGCACTGACACAACCAGCCAGCTGTCCTACGAGAA
CTATGAGAAGTTCAACTCCCATCCCTTCCCTGGGGCAGCTGGGTACCCCACCTACCGACTGGGCTACC
CCCAGGCCCCACCCTCTGGCCTGGAGCGGACCCCATATGAGGCGTATGACCCCATTGGCAAGTACGCC
ACAGCCACTCGATTCTCCTACACCTCCCAGCACTCGGACTACGGCCAGCGATTCCAGCAGCGCATGCA
GACTCACGTGTAGGGGCCAG

| | | | |
|---|---|---|---|
| NOV12v, SNP13374856 of | SEQ ID NO: 362 | 696 aa | MW at 76956.2kD |
| CG51373-08, Protein | SNP Pos: 463 | | SNP Change: Arg to His |
| Sequence | | | |

MGQGLKAWPRYRVVGSADAGQYNLEITDAELSDDASYECQATEAALRSRPAKLTVLIPPEDTRIDGGP
VILLQAGTPHNLTCRAFNAKPAATIIWFRDGTQQEGAVASTELLKDGKRETTVSQLLINPTDLDIGRV
FTCRSMNEAIPSGKETSIELDVHHPPTVTLSIEPQTVQEGERVVFTCQATANPEILDYRWAKGGFLIE
DAHESRYETNVDYSFFTEPVSCEVHNKVGSTNVSTLVNVHFAPRIVVDPKPTTTDIGSDVTLTCVWVG
NPPLTLTWTKKDSNMVLSNSNQLLLKSVTQADAGTYTCEAIVPRIGVAEREVPLYVNGPPIISSEAVQ
YAVRGDGGKVECFIGSTPPPDRIAWAWKENFLEVGTLERYTVERTNSGSGVLSTLTINNVMEADFQTH
YNCTAWNSFGPGTAIIQLEEREVLPVGIIAGATIGASILLIFFFIALVFFLYRRHKGSRKDVTLRKLD
IKVETVNREPLTMHSDREDDTASVSTATRVMKAIYSSFMDDVDLKQDLRCDTIDTREEYEMKDPTNGY
YNVRAHEDRPSSRAVLYADYRAPGPARFDGRPSSRLSHSSGYAQLNTYSRGPASDYGPEPTPPGPAAP
AGTDTTSQLSYENYEKFNSHPFPGAAGYPTYRLGYPQAPPSGLERTPYEAYDPIGKYATATRFSYTSQ
HSDYGQRFQQRMQTHV

| | | |
|---|---|---|
| NOV12w, SNP13374855 of | SEQ ID NO: 363 | 2128 bp |
| CG51373-08, DNA Sequence | ORF Start: ATG at 31 | ORF Stop: TAG at 2119 |
| | SNP Pos: 1421 | SNP Change: A to G |

CAATGGACCAAGGACGGGCTGGCCCTGGGCATGGGCCAGGGCCTCAAAGCCTGGCCACGGTACCGGGT
TGTGGGCTCCGCAGACGCTGGGCAGTACAACCTGGAGATCACAGATGCTGAGCTCTCTGACGACGCCT
CTTACGAGTGCCAGGCCACGGAGGCCGCCCTGCGCTCTCGGCGGGCCAAACTCACCGTGCTCATCCCC
CCAGAGGACACCAGGATTGACGGAGGCCCTGTGATTCTACTGCAGGCAGGCACCCCCCACAACCTCAC
ATGCCGGGCCTTCAATGCGAAGCCTGCTGCCACCATCATCTGGTTCCGGGACGGGACGCAGCAGGAGG
GCGCTGTGGCCAGCACGGAATTGCTGAAGGATGGGAAGAGGGAGACCACCGTGAGCCAACTGCTTATT
AACCCCACGGACCTGGACATAGGGCGTGTCTTCACTTGCCGAAGCATGAACGAAGCCATCCCTAGTGG
CAAGGAGACTTCCATCGAGCTGGATGTGCACCACCCTCCTACAGTGACCCTGTCCATTGAGCCACAGA
CGGTGCAGGAGGGTGAGCGTGTTGTCTTTACCTGCCAGGCCACAGCCAACCCCGAGATCTTGGACTAC
AGGTGGGCCAAAGGGGGTTTCTTGATTGAAGACGCCCACGAGAGTCGCTATGAGACAAATGTGGATTA
TTCCTTTTTCACGGAGCCTGTGTCTTGTGAGGTTCACAACAAAGTGGGAAGCACCAATGTCAGCACTT
TAGTAAATGTCCACTTTGCTCCCCGGATTGTAGTTGACCCCAAACCCACAACCACAGACATTGGCTCT
GATGTGACCCTTACCTGTGTCTGGGTTGGGAATCCCCCCCTCACTCTCACCTGGACCAAAAAGGACTC
AAATATGGTCCTGAGTAACAGCAACCAGCTGCTGCTGAAGTCGGTGACTCAGGCAGACGCTGGCACCT
ACACCTGCCGGGCCATCGTGCCTCGAATCGGAGTGGCTGAGCGGGAGGTGCCGCTCTATGTGAACGGG
CCCCCCATCATCTCCAGTGAGGCAGTGCAGTATGCTGTGAGGGGTGACGGTGGCAAGGTGGAGTGTTT
CATTGGGAGCACACCACCCCCAGACCGCATAGCATGGGCCTGGAAGGAGAACTTCTTGGAGGTGGGGA
CCCTGGAACGCTATACAGTGGAGAGGACCAACTCAGGCAGTGGGGTGCTATCCACGCTCACCATCAAC
AATGTCATGGAGGCCGACTTTCAGACTCACTACAACTGCACCGCCTGGAACAGCTTCGGGCCAGGCAC
AGCCATCATCCAGCTGGAAGAGCGAGAGGTGTTACCTGTGGGCATCATAGCTGGGGCCACCATCGGCG
CGAGCATCCTGCTCATCTTCTTCTTCATCGCCTTGGTATTCTTCCTCTACCGGCGCCGCAGAGGCAGT
CGCAAAGACGTGACCCTGAGGAAGCTGGATATCAAGGTGGAGACAGTGAACCGAGAGCCACTTACGAT
GCATTCTGACCGGGAGGATGACACCGCCAGCGTCTCCACAGCAACCCGGGTCATGAAGGCCATCTACT
CGTCGTTTATGGATGATGTGGATCTGAAGCAGGACCTGCGCTGCGACACCATCGACACCCGGGAGGAG
TATGAGATGAAGGACCCCACCAATGGCTACTACAACGTGCGTGCCCATGAAGACCGCCCGTCTTCCAG
GGCAGTGCTCTATGCTGACTACCGTGCCCCTGGCCCTGCCCGCTTCGACGGCCGCCCCTCATCCCGTC
TCTCCCACTCCAGCGGCTATGCCCAGCTCAACACCTATAGCCGGGGCCCTGCCTCTGACTATGGCCCT
GAGCCCACACCCCCTGGCCCTGCTGCCCCAGCTGGCACTGACACAACCAGCCAGCTGTCCTACGAGAA
CTATGAGAAGTTCAACTCCCATCCCTTCCCTGGGGCAGCTGGGTACCCCACCTACCGACTGGGCTACC
CCCAGGCCCCACCCTCTGGCCTGGAGCGGACCCCATATGAGGCGTATGACCCCATTGGCAAGTACGCC
ACAGCCACTCGATTCTCCTACACCTCCCAGCACTCGGACTACGGCCAGCGATTCCAGCAGCGCATGCA
GACTCACGTGTAGGGGCCAG

| | | | |
|---|---|---|---|
| NOV12w, SNP13374855 of | SEQ ID NO: 364 | 696 aa | MW at 77003.3kD |
| CG51373-08, Protein | SNP Pos: 464 | | SNP Change: Lys to Arg |
| Sequence | | | |

MGQGLKAWPRYRVVGSADAGQYNLEITDAELSDDASYECQATEAALRSRRAKLTVLIPPEDTRIDGGP
VILLQAGTPHNLTCRAFNAKPAATIIWFRDGTQQEGAVASTELLKDGKRETTVSQLLINPTDLDIGRV
FTCRSMNEAIPSGKETSIELDVHHPPTVTLSIEPQTVQEGERVVFTCQATANPEILDYRWAKGGFLIE
DAHESRYETNVDYSFFTEPVSCEVHNKVGSTNVSTLVNVHFAPRIVVDPKPTTTDIGSDVTLTCVWVG
NPPLTLTWTKKDSNMVLSNSNQLLLKSVTQADAGTYTCRAIVPRIGVAEREVPLYVNGPPIISSEAVQ
YAVRGDGGKVECFIGSTPPPDRIAWAWKENFLEVGTLERYTVERTNSGSGVLSTLTINNVMEADFQTH
YNCTAWNSFGPGTAIIQLEEREVLPVGIIAGATIGASILLIFFFIALVFFLYRRRRGSRKDVTLRKLD
IKVETVNREPLTMHSDREDDTASVSTATRVMKAIYSSFMDDVDLKQDLRCDTIDTREEYEMKDPTNGY
YNVRAEEDRPSSEAVLYADYRAPGPARFDGRPSSRLSHSSGYAQLNTYSRGPASDYGPEPTPPGPAAP
AGTDTTSQLSYENYEKFNSHPFPGAAGYPTYRLGYPQAPPSGLERTPYEAYDPIGKYATATRFSYTSQ
HSDYGQRFQQRMQTHVJ

| | | |
|---|---|---|
| NOV12x, SNP13374854 of | SEQ ID NO: 365 | 2128 bp |
| CG51373-08, DNA Sequence | ORF Start: ATG at 31 | ORF Stop: TAG at 2119 |
| | SNP Pos: 1426 | SNP Change: A to T |

CAATGGACCAAGGACGGGCTGGCCCTGGGCATGGGCCAGGGCCTCAAAGCCTGGCCACGGTACCGGGT
TGTGGGCTCCGCAGACGCTGGGCAGTACAACCTGGAGATCACAGATGCTGAGCTCTCTGACGACGCCT
CTTACGAGTGCCAGGCCACGGAGGCCGCCCTGCGCTCTCGGCGGGCCAAACTCACCGTGCTCATCCCC
CCAGAGGACACCAGGATTGACGGAGGCCCTGTGATTCTACTGCAGGCAGGCACCCCCCACAACCTCAC
ATGCCGGGCCTTCAATGCGAAGCCTGCTGCCACCATCATCTGGTTCCGGGACGGGACGCAGCAGGAGG
GCGCTGTGGCCAGCACGGAATTGCTGAAGGATGGGAAGAGGGAGACCACCGTGAGCCAACTGCTTATT
AACCCCACGGACCTGGACATAGGGCGTGTCTTCACTTGCCGAAGCATGAACGAAGCCATCCCTAGTGG
CAAGGAGACTTCCATCGAGCTGGATGTGCACCACCCTCCTACAGTGACCCTGTCCATTGAGCCACAGA
CGGTGCAGGAGGGTGAGCGTGTTGTCTTTACCTGCCAGGCCACAGCCAACCCCGAGATCTTGGACTAC
AGGTGGGCCAAAGGGGGTTTCTTGATTGAAGACGCCCACGAGAGTCGCTATGAGACAAATGTGGATTA
TTCCTTTTTCACGGAGCCTGTGTCTTGTGAGGTTCACAACAAAGTGGGAAGCACCAATGTCAGCACTT
TAGTAAATGTCCACTTTGCTCCCCGGATTGTAGTTGACCCCAAACCCACAACCACAGACATTGGCTCT
GATGTGACCCTTACCTGTGTCTGGGTTGGGAATCCCCCCCTCACTCTCACCTGGACCAAAAAGGACTC
AAATATGGTCCTGAGTAACAGCAACCAGCTGCTGCTGAAGTCGGTGACTCAGGCAGACGCTGGCACCT
ACACCTGCCGGGCCATCGTGCCTCGAATCGGAGTGGCTGAGCGGGAGGTGCCGCTCTATGTGAACGGG
CCCCCCATCATCTCCAGTGAGGCAGTGCAGTATGCTGTGAGGGGTGACGGTGGCAAGGTGGAGTGTTT
CATTGGGAGCACACCACCCCCAGACCGCATAGCATGGGCCTGGAAGGAGAACTTCTTGGAGGTGGGGA
CCCTGGAACGCTATACAGTGGAGAGGACCAACTCAGGCAGTGGGGTGCTATCCACGCTCACCATCAAC
AATGTCATGGAGGCCGACTTTCAGACTCACTACAACTGCACCGCCTGGAACAGCTTCGGGCCAGGCAC
AGCCATCATCCAGCTGGAAGAGCGAGAGGTGTTACCTGTGGGCATCATAGCTGGGGCCACCATCGGCG
CGAGCATCCTGCTCATCTTCTTCTTCATCGCCTTGGTATTCTTCCTCTACCGGCGCCGCAAAGGCTGT
CGCAAAGACGTGACCCTGAGGAAGCTGGATATCAAGGTGGAGACAGTGAACCGAGAGCCACTTACGAT
GCATTCTGACCGGGAGGATGACACCGCCAGCGTCTCCACAGCAACCCGGGTCATGAAGGCCATCTACT
CGTCGTTTATGGATGATGTGGATCTGAAGCAGGACCTGCGCTGCGACACCATCGACACCCGGGAGGAG
TATGAGATGAAGGACCCCACCAATGGCTACTACAACGTGCGTGCCCATGAAGACCGCCCGTCTTCCAG
GGCAGTGCTCTATGCTGACTACCGTGCCCCTGGCCCTGCCCGCTTCGACGGCCGCCCCTCATCCCGTC
TCTCCCACTCCAGCGGCTATGCCCAGCTCAACACCTATAGCCGGGGCCCTGCCTCTGACTATGGCCCT
GAGCCCACACCCCCTGGCCCTGCTGCCCCAGCTGGCACTGACACAACCAGCCAGCTGTCCTACGAGAA
CTATGAGAAGTTCAACTCCCATCCCTTCCCTGGGGCAGCTGGGTACCCCACCTACCGACTGGGCTACC
CCCAGGCCCCACCCTCTGGCCTGGAGCGGACCCCATATGAGGCGTATGACCCCATTGGCAAGTACGCC
ACAGCCACTCGATTCTCCTACACCTCCCAGCACTCGGACTACGGCCAGCGATTCCAGCAGCGCATGCA
GACTCACGTGTAGGGGCCAG

| | | | |
|---|---|---|---|
| NOV12x, SNP13374854 of | SEQ ID NO: 366 | 696 aa | MW at 76991.4kD |
| CG51373-08, Protein | SNP Pos: 466 | | SNP Change: Ser to Cys |
| Sequence | | | |

MGQGLKAWPRYRVVGSADAGQYNLEITDAELSDDASYECQATEAALRSRRAKLTVLIPPEDTRIDGGP
VILLQAGTPHNLTCRAFNAKPAATIIWFRDGTQQEGAVASTELLKDGKRETTVSQLLINPTDLDIGRV
FTCRSMNEAIPSGKETSIELDVHHPPTVTLSIEPQTVQEGERVVFTCQATANPEILDYRWAKGGFLIE
DAHESRYETNVDYSFFTEPVSCEVHNKVGSTNVSTLVNVHFAPRIVVDPKPTTTDIGSDVTLTCVWVG
NPPLTLTWTKKDSNMVLSNSNQLLLKSVTQADAGTYTCRAIVPRIGVAEREVPLYVNGPPIISSEAVQ
YAVRGDGGKVECFIGSTPPPDRIAWAWKENFLEVGTLERYTVERTNSGSGVLSTLTINNVMEADFQTH
YNCTAWNSFGPGTAIIQLEEREVLPVGIIAGATIGASILLIFFFIALVFFLYRRRKGCRKDVTLRKLD
IKVETVNREPLTMHSDREDDTASVSTATRVMKAIYSSFMDDVDLKQDLRCDTIDTREEYEMKDPTNGY
YNVRAHEDRPSSRAVLYADYRAPGPARFDGRPSSRLSHSSGYAQLNTYSRGPASDYGPEPTPPGPAAP
AGTDTTSQLSYENYEKFNSHPFPGAAGYPTYRLGYPQAPPSGLERTPYEAYDPIGKYATATRFSYTSQ
HSDYGQRFQQRMQTHV

| | | |
|---|---|---|
| NOV12y, SNP13374853 of | SEQ ID NO: 367 | 2128 bp |
| CG51373-08, DNA Sequence | ORF Start: ATG at 31 | ORF Stop: TAG at 2119 |
| | SNP Pos: 1436 | SNP Change: A to G |

CAATGGACCAAGGACGGGCTGGCCCTGGGCATGGGCCAGGGCCTCAAAGCCTGGCCACGGTACCGGGT
TGTGGGCTCCGCAGACGCTGGGCAGTACAACCTGGAGATCACAGATGCTGAGCTCTCTGACGACGCCT
CTTACGAGTGCCAGGCCACGGAGGCCGCCCTGCGCTCTCGGCGGGCCAAACTCACCGTGCTCATCCCC
CCAGAGGACACCAGGATTGACGGAGGCCCTGTGATTCTACTGCAGGCAGGCACCCCCCACAACCTCAC
ATGCCGGGCCTTCAATGCGAAGCCTGCTGCCACCATCATCTGGTTCCGGGACGGGACGCAGCAGGAGG
GCGCTGTGGCCAGCACGGAATTGCTGAAGGATGGGAAGAGGGAGACCACCGTGAGCCAACTGCTTATT
AACCCCACGGACCTGGACATAGGGCGTGTCTTCACTTGCCGAAGCATGAACGAAGCCATCCCTAGTGG
CAAGGAGACTTCCATCGAGCTGGATGTGCACCACCCTCCTACAGTGACCCTGTCCATTGAGCCACAGA
CGGTGCAGGAGGGTGAGCGTGTTGTCTTTACCTGCCAGGCCACAGCCAACCCCGAGATCTTGGACTAC
AGGTGGGCCAAAGGGGGTTTCTTGATTGAAGACGCCCACGAGAGTCGCTATGAGACAAATGTGGATTA
TTCCTTTTTCACGGAGCCTGTGTCTTGTGAGGTTCACAACAAAGTGGGAAGCACCAATGTCAGCACTT
TAGTAAATGTCCACTTTGCTCCCCGGATTGTAGTTGACCCCAAACCCACAACCACAGACATTGGCTCT
GATGTGACCCTTACCTGTGTCTGGGTTGGGAATCCCCCCCTCACTCTCACCTGGACCAAAAAGGACTC
AAATATGGTCCTGAGTAACAGCAACCAGCTGCTGCTGAAGTCGGTGACTCAGGCAGACGCTGGCACCT
ACACCTGCCGGGCCATCGTGCCTCGAATCGGAGTGGCTGAGCGGGAGGTGCCGCTCTATGTGAACGGG
CCCCCCATCATCTCCAGTGAGGCAGTGCAGTATGCTGTGAGGGGTGACGGTGGCAAGGTGGAGTGTTT
CATTGGGAGCACACCACCCCCAGACCGCATAGCATGGGCCTGGAAGGAGAACTTCTTGGAGGTGGGGA
CCCTGGAACGCTATACAGTGGAGAGGACCAACTCAGGCAGTGGGGTGCTATCCACGCTCACCATCAAC
AATGTCATGGAGGCCGACTTTCAGACTCACTACAACTGCACCGCCTGGAACAGCTTCGGGCCAGGCAC
AGCCATCATCCAGCTGGAAGAGCGAGAGGTGTTACCTGTGGGCATCATAGCTGGGGCCACCATCGGCG
CGAGCATCCTGCTCATCTTCTTCTTCATCGCCTTGGTATTCTTCCTCTACCGGCGCCGCAAAGGCAGT
CGCAAAGGCGTGACCCTGAGGAAGCTGGATATCAAGGTGGAGACAGTGAACCGAGAGCCACTTACGAT
GCATTCTGACCGGGAGGATGACACCGCCAGCGTCTCCACAGCAACCCGGGTCATGAAGGCCATCTACT
CGTCGTTTATGGATGATGTGGATCTGAAGCAGGACCTGCGCTGCGACACCATCGACACCCGGGAGGAG
TATGAGATGAAGGACCCCACCAATGGCTACTACAACGTGCGTGCCCATGAAGACCGCCCGTCTTCCAG
GGCAGTGCTCTATGCTGACTACCGTGCCCCTGGCCCTGCCCGCTTCGACGGCCGCCCCTCATCCCGTC
TCTCCCACTCCAGCGGCTATGCCCAGCTCAACACCTATAGCCGGGGCCCTGCCTCTGACTATGGCCCT
GAGCCCACACCCCCTGGCCCTGCTGCCCCAGCTGGCACTGACACAACCAGCCAGCTGTCCTACGAGAA
CTATGAGAAGTTCAACTCCCATCCCTTCCCTGGGGCAGCTGGGTACCCCACCTACCGACTGGGCTACC
CCCAGGCCCCACCCTCTGGCCTGGAGCGGACCCCATATGAGGCGTATGACCCCATTGGCAAGTACGCC
ACAGCCACTCGATTCTCCTACACCTCCCAGCACTCGGACTACGGCCAGCGATTCCAGCAGCGCATGCA
GACTCACGTGTAGGGGCCAG

| | | | |
|---|---|---|---|
| NOV12y, SNP13374853 of | SEQ ID NO: 368 | 696 aa | MW at 76917.3kD |
| CG51373-08, Protein | SNP Pos: 469 | | SNP Change: Asp to Gly |
| Sequence | | | |

MGQGLKAWPRYRVVGSADAGQYNLEITDAELSDDASYECQATEAALRSRRAKLTVLIPPEDTRIDGGP
VILLQAGTPHNLTCRAFNAKPAATIIWFRDGTQQEGAVASTELLKDGKRETTVSQLLINPTDLDIGRV
FTCRSNNEAIPSGKETSIELDVHHPPTVTLSIEPQTVQEGERVVFTCQATANPEILDYRWAKGGFLIE
DAHESRYETNVDYSFFTEPVSCEVHNKVGSTNVSTLVNVHFAPRIVVDPKPTTTDIGSDVTLTCVWVG
NPPLTLTWTKKDSNMVLSNSNQLLLKSVTQADAGTYTCRAIVPRIGVAEREVPLYVNGPPIISSEAVQ
YAVRGDGGKVECFIGSTPPPDRIAWAWKENFLEVGTLERYTVERTNSGSGVLSTLTINNVMEADFQTH
YNCTAWNSFGPGTAIIQLEEREVLPVGIIAGATIGASILLIFFFIALVFFLYRRRKGSRKGVTLRKLD
IKVETVNREPLTMHSDREDDTASVSTATRVMKAIYSSFMDDVDLKQDLRCDTIDTREEYEMKDPTNGY
YNVEAHEDRPSSRAVLYADYRAPGPARFDGRPSSRLSHSSGYAQLNTYSRGPASDYGPEPTPPGPAAP
AGTDTTSQLSYENYEKFNSHPFPGAAGYPTYRLGYPQAPPSGLERTPYEAYDPIGKYATATRFSYTSQ
HSDYGQRFQQRMQTHV

A ClustalW comparison of the above protein sequences yields the following sequence alignment shown in Table 12B.

TABLE 12B
Comparison of the NOV12 protein sequences.

NOV12a ------------------------------------------------------------
NOV12b ------------------------------------------------------------
NOV12c ------------------------------------------------------------
NOV12d ------------------------------------------------------------
NOV12e ------------------------------------------------------------
NOV12f ------------------------------------------------------------
NOV12g MHLTLEVLNHGPFPLNLSSIAYNHGTVFGHWKNNVTRETLVKVKDAEDQLGARVGYIELD
NOV12h MHLTLEVLNHGPFPLNLSSIAYNHGTVFGHWKNNVTRETLVKVKDAEDQLGARVGYIELD
NOV12i ------------------------------------------------------------
NOV12j ------------------------------------------------------------
NOV12k ------------------------------------------------------------
NOV12l ------------------------------------------------------------
NOV12m ------------------------------------------------------------
NOV12n ------------------------------------------------------------
NOV12o ------------------------------------------------------------
NOV12p ------------------------------------------------------------
NOV12q ------------------------------------------------------------
NOV12a -------------------------MLSLLVWILTLSDTFSQGTQTRFSQEPADQWVAGQ
NOV12b ------------------------------------------------------------
NOV12c ------------------------------------------------------------
NOV12d ------------------------------------------------------------
NOV12e ---------------------TKLMLSLLVWILTLSDTFSQGTQTRFSQEPADQTVVAGQ
NOV12f ---------------------TKLMLSLLVWILTLSDTFSQGTQTRFSQEPADQTVVAGQ
NOV12g LNSGKETFLVNEEATGETSGDNVVBSRNLSQTIFITRKRWEGTQTRFSQEPADQTVVAGQ
NOV12h LNSGKETFLVNEEATGETSGDNVVHSRNLSQTIFITRKRWEGTQTRFSQEPADQTVVAGQ
NOV12i ------------------------------------------------------------
NOV12j ------------------------------------------------------------
NOV12k ------------------------------------------------------------
NOV12l ------------------------------------------------------------
NOV12m ---------------------------------------------RFSQEPADQTVVAGQ
NOV12n ------------------------------------------------------------
NOV12o ------------------------MLSLLVWILTLSDTFSQGTQTRFSQEPADQTVVAGQ
NOV12p ------------------------MLSLLVWILTLSDTFSQGTQTRFSQEPADQTVVAGQ
NOV12q ----------------------------------------QGTQTRFSQEPADQTVVAGQ
NOV12a RAVLPCVLLNYSGIVQWTKDGLALGMGQGLKAWPRYRVVGSADAGQYNLEITDAELSDDA
NOV12b -------------------------MGQALKAWPRYRVVGSADAGQYNLEITDAELSDDA
NOV12c -------------------------MGQGLKAWPRYRVVGSADAGQYNLEITDAELSDDA
NOV12d -----------------------GSMGQGLKAWPRYRVVGSADAGQYNLEITDAELSDDA
NOV12e RAVLPCVLLNYSGIVQWTKDGLALGMGQGLKAWPRYRVVGSADAGQYNLEITDAELSDDA
NOV12f RAVLPCVLLNYSGIVQWTKDGLALGMGQGLKAWPRYRVVGSADAGQYNLEITDAELSDDA
NOV12g RAVLPCVLLNYSGIVQWTKDGLALGMGQALKAWPRYRVVGSADAGQYNLEITDAELSDDA
NOV12h RAVLPCVLLNYSGIVQWTKDGLALGMGQALKAWPRYRVVGSADAGQYNLEITDAELSDDA
NOV12i -------------------------MGQALKAWPRYRVVGSADAGQYNLEITDAELSDDA
NOV12j -------------------------MGQALKAWPRYRVVGSADAGQYNLEITDAELSDDA
NOV12k -------------------------MGQALKAWPRYRVVGSADAGQYNLEITDAELSDDA
NOV12l -------------------------MGQALKAWPRYRVVGSADAGQYNLEITDAELSDDA
NOV12m RAVLPCVLLNYSGIVQWTKDGLALGMGQALKAWPRYRVVGSADAGQYNLEITDAELSDDA
NOV12n -------------------------MGQGLKAWPRYRVVGSADAGQYNLEITDAELSDDA
NOV12o RAVLPCVLLNYSGIVQWTKDGLALGMGQGLKAWPRYRVVGSADAGQYNLEITDAELSDDA
NOV12p RAVLPCVLLNYSGIVQWTKDGLALGMGQGLKAWPRYRVVGSADAGQYNLEITDAELSDDA
NOV12q RAVLPCVLLNYSGIVQWTKDGLALGMGQGLKAWPRYRVVGSADAGQYNLEITDAELSDDA
NOV12a SYECQATEAALRSRRAKLTVLIPPEDTRIDGGPVILLQAGTPHNLTCRAFNAKPAATIIW
NOV12b SYECQATEAALRSRRAKLTVLIPPEDTRIDGGPVILLQAGTPHNLTCRAFNAKPAATIIW
NOV12c SYECQATEAALRSRRAXLTVLIPPEDTRIDGGPVILLQAGTPHNLTCRAFNAKPAATIIW
NOV12d SYECQATEAALRSRRAKLTVLIPPEDTRIDGGPVILLQAGTPHNLTCEAFNAKPAATIIW
NOV12e SYECQATEAALRSRRAKLTVLIPPEDTRIDGGPVILLQAGTPHNLTCEAFNAKPAATIIW
NOV12f SYECQATEAALRSRRAKLTVLIPPEDTRIDGGPVILLQAGTPHNLTCRAFNAKPAATIIW
NOV12g SYECQATEAALRSRRAXLTVLIPPEDTRIDGGPVILLQAGTPHNLTCRAFNAKPAATIIW
NOV12h SYECQATEAALRSRRAKLTVLIPPEDTRIDGGPVILLQAGTPHNLTCEAFNAKPAATIIW
NOV12i SYECQATEAALRSRRAKLTVLIPPEDTRIDGGPVILLQAGTPHNLTCRAFNAKPAATIIW
NOV12j SYECQATEAALRSRRAKLTVLIPPEDTRIDGGPVILLQAGTPHNLTCRAFNAKPAATIIW
NOV12k SYECQATEAALRSRPAKLTVLIPPEDTRIDGGPVILLQAGTPHNLTCRAFNAKPAATIIW
NOV12l SYECQATEAALRSRRAKLTVLIPPEDTRIDGGPVILLQAGTPHNLTCRAFNAKPAATIIW
NOV12m SYECQATEAALRSRRAKLTVLIPPEDTRIDGGPVILLQAGTPHNLTCRAFNAKPAATIIW
NOV12n SYECQATEAALRSRRAKLTVLIPPEDTRIDGGPVILLQAGTPHNLTCRAFNAKPAATIIW
NOV12o SYECQATEAALRSRRAKLTVLIPPEDTRIDGGPVILLQAGTPHNLTCRAFNAKPAATIIW
NOV12p SYECQATEAALRSRRAKLTVLIPPEDTRIDGGPVILLQAGTPHNLTCHAFNAKPAATIIW
NOV12q SYECQATEAALRSRRAKLTVLIPPEDTRIDGGPVILLQAGTPHNLTCRAFNAKPAATIIW
NOV12a FRDGTQQEGAVASTELLKDGKRETTVSQLLINPTDLDIGRVFTCRSMNEAIPSGKETSIE
NOV12b FRDGTQQEGAVASTELLKDGKRETTVSQLLINPTDLDIGRVFTCRSMNEAIPSGKETSIE
NOV12c FRDGTQQEGAVASTELLKDGKRETTVSQLLINPTDLDIGRVFTCRSMNEAIPSGKETSIE
NOV12d FRDGTQQEGAVASTELLKDGKRETTVSQLLINPTDLDIGRVFTCRSMNEAIPSGKETSIE
NOV12e FRDGTQQEGAVASTELLKDGKRETTVSQLLINPTDLDIGRVFTCRSMNEAIPSGKETSIE
NOV12f FRDGTQQEGAVASTELLKDGKRETTVSQLLINPTDLDIGRVFTCRSMNEAIPSGKETSIE
NOV12g FRDGTQQEGAVASTELLKDGKRETTVSQLLINPTDLDIGRVFTCRSMNEAIPSGKETSIE
NOV12h FRDGTQQEGAVASTELLKDGKRETTVSQLLINPTDLDIGRVFTCRSMNEAIPSGKETSIE
NOV12i FRDGTQQEGAVASTELLKDGKRETTVSQLLINPTDLDIGRVFTCRSMNEAIPSGKETSIE
NOV12j FRDGTQQEGAVASTELLKDGKRETTVSQLLINPTDLDIGRVFTCRSMNEAIPSGKETSIE
NOV12k FRDGTQQEGAVASTELLKDGKRETTVSQLLINPTDLDIGRVFTCRSMNEAIPSGKETSIE
NOV12l FRDGTQQEGAVASTELLKDGKRETTVSQLLINPTDLDIGRVFTCRSMNEAIPSGKETSIE
NOV12m FRDGTQQEGAVASTELLKDGKRETTVSQLLINPTDLDIGRVFTCRSMNEAIPSGKETSIE
NOV12n FRDGTQQEGAVASTELLKDGKRETTVSQLLINPTDLDIGRVFTCRSNNEAIPSGKETSIE
NOV12o FRDGTQQEGAVASTELLKDGKRETTVSQLLINPTDLDIGRVFTCRSMNEAIPSGKETSIE
NOV12p FRDGTQQEGAVASTELLKDGKRETTVSQLLINPTDLDIGRVFTCRSMNEAIPSGKETSIE
NOV12q FRDGTQQEGAVASTELLKDGKRETTVSQLLINPTDLDIGRVFTCRSMNEAIPSGKETSIE
NOV12a LDVHHPPTVTLSIEPQTVQEGERVVFTCQATANPEILGYRWAKGGFLIEDAHESRYETNV
NOV12b LDVHHPPTVTLSIEPQTVQEGERVVFTCQATANPEILGYRWAKGGFLIEDAHESRYETNV
NOV12c LDVHHPPTVTLSIEPQTVQEGERVVFTCQATANPEILDYRWAKGGFLIEDAHESRYETNV
NOV12d LDVHHPPTVTLSIEPQTVQEGERVVFTCQATANPEILDYRWAKGGFLIEDAHESRYETNV
NOV12e LDVHHPPTVTLSIEPQTVQEGERVVFTCQATANPEILGYRWAKGGFLIEDAHESRYETNV
NOV12f LDVHHPPTVTLSIEPQTVQEGERVVFTCQATANPEILGYRWAKGGFLIEDAHESRYETNV
NOV12g LDVHREWAGGSSLEQGGGRRGVFEKHTLSLRNTN--------------------------
NOV12h LDVHHPPTVTLSIEPQTVQEGERVVFTCQATANPEILGYRWAKGGFLIEDAHESRYETNV
NOV12i LDVHHPPTVTLSIEPQTVQEGERVVFTCQATANPEILGYRWAKGGFLIEDAHESRYETNV
NOV12j LDVHREWAGGSSLEQGGGRRGVFEKHTLSLRNTN--------------------------
NOV12k LDVHREWAGGSSLEQGGGRRGVFEKHTLSLRNTN--------------------------
NOV12l LDVHREWAGGSSLEQGGGRRGVFEKHTLSLRNTN--------------------------
NOV12m LDVHHPPTVTLSIEPQTVQEGERVVFTCQATANPEILGYRWAKGGFLIEDAHESRYETNV
NOV12n LDVHHPPTVTLSIEPQTVQEGERVVFTCQATANPEILDYRWAKGGFLIEDAHESRYETNV
NOV12o LDVHHPPTVTLSIEPQTVQEGERVVFTCQATANPEILGYRWAKGGFLIEDAHESRYETNV
NOV12p LDVHHPPTVTLSIEPQTVQEGERVVFTCQATANPEILGYRWAKGGFLIEDAHESRYETNV
NOV12q LDVHHPPTVTLSIEPQTVQEGERVVFTCQATANPEILGYRWAKGGFLIEDAHESRYETNV
NOV12a DYSFFTEPVSCEVHNKVGSTNVSTLVNVHFAPRIVVDPKPTTTDIGSDVTLTCVWVGNPP
NOV12b DYSFFTEPVSCEVHNKVGSTNVSTLVNVHFAPRIVVDPKPTTTDIGSDVTLTCVWVGEIP
NOV12c DYSFFTEPVSCEVHNKVGSTNVSTLVNVHFAPRIVVDPKPTTTDIGSDVTLTCVWVGNPP
NOV12d DYSFFTEPVSCEVHNKVGSTNVSTLVNVHFAPRIVVDPKPTTTDIGSDVTLTCVWVGNPP
NOV12e DYSFFTEPVSCEVHNKVGSTNVSTLVNVHFAPRIVVDPKPTTTDIGSDVTLTCVWVGNPP
NOV12f DYSFFTEPVSCEVHNKVGSTNVSTLVNVHFAPRIVVDPKPTTTDIGSDVTLTCVWVGNPP
NOV12g ------------------------------------------------------------
NOV12h DYSFFTEPVSCEVHNKVGSTNVSTLVNVHFAPRIVVDPKPTTTDIGSDVTLTCVWVGNPP
NOV12i DYSFFTEPVSCEVHNKVGSTNVSTLVNVHFAPRIVVDPKPTTTDIGSDVTLTCVWVGNPP
NOV12j ------------------------------------------------------------
NOV12k ------------------------------------------------------------
NOV12l ------------------------------------------------------------
NOV12m DYSFFTEPVSCEVHNKVGSTNVSTLVNVHFAPRIVVDPKPTTTDIGSDVTLTCVWVGNPP
NOV12n DYSFFTEPVSCEVHNKVGSTNVSTLVNVHFAPRIVVDPKPTTTDIGSDVTLTCVWVGNPP
NOV12o DYSFFTEPVSCEVHNKVGSTNVSTLVNVHFAPRIVVDPKPTTTDIGSDVTLTCVWVGNPP
NOV12p DYSFFTEPVSCEVHNKVGSTNVSTLVNVHFAPRIVVDPKPTTTDIGSDVTLTCVWVGNPP
NOV12q DYSFFTEPVSCEVHNKVGSTNVSTLVNVHFAPRIVVDPKPTTTDIGSDVTLTCVWVGNPP
NOV12a LTLTWTKKD-----------------SNMVLSNSNQLLLKSVTQADAGTYTCPAIVPRIG
NOV12b PSLSPGPKRTQILGPWLLGSPPEAALSAQVLSNSNQLLLKSVTQADAGTYTCRAIVPRIG
NOV12c LTLTWTKKD-----------------SNMVLSNSNQLLLKSVTQADAGTYTCRAIVPRIG
NOV12d LTLTWTKKD-----------------SNMVLSNSNQLLLKSVTQADAGTYTCRAIVPRIG
NOV12e LTLTWTKKD-----------------SNMVLSNSNQLLLKSVTQADAGTYTCRAIVPRIG
NOV12f LTLTWTKKDSN-MGPRPPGSPPEAALSAQVLSNSNQLLLKSVTQADAGTYTCPAIVPRIG
NOV12g ------------------------------------------------------------
NOV12h LTLTWTKKD-----------------SNMVLSNSNQLLLKSVTQADAGTYTCRAIVPRIG
NOV12i LTLTWTKK-----------------DSNMVLSNSNQLLLKSVTQADAGTYTCRAIVPRIG
NOV12j ------------------------------------------------------------
NOV12k ------------------------------------------------------------
NOV12l ------------------------------------------------------------
NOV12m LTLTWTKKD-----------------SNMVLSNSNQLLLKSVTQADAGTYTCRATVPRIG
NOV12n LTLTWTKKD-----------------SNMVLSNSNQLLLKSVTQADAGTYTCRAIVPRIG
NOV12o LTLTWTKKDSN-MGPRPPGSPPEAALSAQVLSNSNQLLLKSVTQADAGTYTCRAIVPRIG
NOV12p LTLTWTKKD-----------------SNMVLSNSNQLLLKSVTQADAGTYTCEAIVPRIG
NOV12q LTLTWTKKDSN-MGPRPPGSPPEAALSAQVLSNSNQLLLKSVTQADAGTYTCRAIVPRIG
NOV12a VAEREVPLYVNGPPIISSEAVQYAVRGDGGKVECFIGSTPPPDRIAWAWKENFLEVGTLE
NOV12b VAEREVPLYVNGPPIISSEAVQYAVRGDGGKVECFIGSTPPPDRIAWAWKENFLEVGTLE
NOV12c VAEREVPLYVNGPPIISSEAVQYAVRGDGGKVECFIGSTPPPDRIAWAWKENFLEVGTLE
NOV12d VAEREVPLYVNGPPIISSEAVQYAVRGDGGKVECFIGSTPPPDRIAWAWKENFLEVGTLE
NOV12e VAEREVPLYVNGPPIISSEAVQYAVRGDGGKVECFIGSTPPPDRIAWAWKENFLEVGTLE
NOV12f VAEREVPLYVNGPPIISSEAVQYAVRGDGGKVECFIGSTPPPDRIAWAWKENFLEVGTLE
NOV12g ------------------------------------------------------------
NOV12h VAEREVPLYVNGPPIISSEAVQYAVRGDGGKVECFIGSTPPPDRIAWAWKENFLEVGTLE
NOV12i VAEREVPLYVNGPPIISSEAVQYAVRGDGGKVECFIGSTPPPDRIAWAWKENFLEVGTLE
NOV12j ------------------------------------------------------------
NOV12k ------------------------------------------------------------
NOV12l ------------------------------------------------------------
NOV12m VAEREVPLYVNGPPIISSEAVQYAVRGDGGKVECFIGSTPPPDRIAWAWKENFLEVGTLE
NOV12n VAEREVPLYVNGPPIISSEAVQYAVRGDGGKVECFIGSTPPPDRIAWAWKENFLEVGTLE
NOV12o VAEREVPLYVNGPPIISSEAVQYAVRGDGGKVECFIGSTPPPDRIAWAWKENFLEVGTLE
NOV12p VAEREVPLYVNGPPIISSEAVQYAVRGDGGKVECFIGSTPPPDRIAWAWKENFLEVGTLE
NOV12q VAEREVPLYVNGPPIISSEAVQYAVRGDGGKVECFIGSTPPPDRIAWAWKENFLEVGTLE
NOV12a RYTVERTNSGSGVLSTLTINNVMEADFQTHYNCTAWNSFGPGTAIIQLEEREVLPVGIIA
NOV12b RYTVERTNSGSGVLSTLTINNVMEADFQTHYNCTAWNSFGPGTAIIQLEEREVLPVGIIA
NOV12c RYTVERTNSGSGVLSTLTINNVMEADFQTHYNCTAWNSFGPGTAIIQLEEREVLPVGIIA
NOV12d RYTVERTNSGSGVLSTLTINNVMEADFQTHYNCTAWNSFGPGTAIIQLEEREVLPVGIIA
NOV12e RYTVERTNSGSGVLSTLTINNVMEADFQTHYNCTAWNSFGPGTAIIQLEEREVLPVGIIA
NOV12f RYTVERTNSGSGVLSTLTINNVMEADFQTHYNCTAWNSFGPGTAIIQLEEREVLPVGIIA
NOV12g ------------------------------------------------------------
NOV12h RYTVERTNSGSGVLSTLTINNVMEADFQTHYNCTAWNSFGPGTAIIQLEEREVLPVGIIA
NOV12i RYTVERTNSGSGVLSTLTINNVMEADFQTHYNCTAWNSFGPGTAIIQLEEREVLPVGIIA
NOV12j ------------------------------------------------------------
NOV12k ------------------------------------------------------------
NOV12l ------------------------------------------------------------
NOV12m RYTVERTNSGSGVLSTLTINNVMEADFQTHYNCTAWNSFGPGTAIIQLEER---------
NOV12n RYTVERTNSGSGVLSTLTINNVMEADFQTHYNCTAWNSFGPGTAIIQLEEREVLPVGIIA
NOV12o RYTVERTNSGSGVLSTLTINNVMEADFQTHYNCTAWNSFGPGTAIIQLEEREVLPVGIIA
NOV12p RYTVERTNSGSGVLSTLTINNVMEADFQTHYNCTAWNSFGPGTAIIQLEEREVLPVGIIA
NOV12q RYTVERTNSGSGVLSTLTINNVMEADFQTHYNCTAWNSFGPGTAIIQLEEREVLPVGIIA
NOV12a GATIGASILLIFFFIALVFFLYRRRKGSRKDVTLRKLDIKVETVNREPLTMHSDREDDTA
NOV12b GATIGASILLIFFFIALVFFLYRRRKGSRKDVTLRKLDIKVETVNREPLTMHSDREDDTA
NOV12c GATIGASILLIFFFIALVFFLYRRRKGSRKDVTLRKLDIKVETVNREPLTMHSDREDDTA
NOV12d GATIGASILLIFFFIALVFFLYRRRKGSRKDVTLRKLDIKVETVNREPLTMHSDREDDTA
NOV12e GATIGASILLIFFFIALVFFLYRRRKGSRKDVTLRKLDIKVETVNREPLTMHSDREDDTA
NOV12f GATIGASILLIFFFIALVFFLYRRRKGSRKDVTLRKLDIKVETVNREPLTMHSDREDDTA
NOV12g ------------------------------------------------------------
NOV12h GATIGASILLIFFFIALVFFLYRRRKGSRKDVTLRKLDIKVETVNREPLTMHSDREDDTA
NOV12i GATIGASILLIFFFIALVFFLYRRRKGSRKDVTLRKLDIKVETVNREPLTMHSDREDDTA
NOV12j ------------------------------------------------------------
NOV12k ------------------------------------------------------------
NOV12l ------------------------------------------------------------
NOV12m ------------------------------------------------------------
NOV12n GATIGASILLIFFFIALVFFLYRRRKGSRKDVTLRKLDIKVETVNREPLTMHSDREDDTA
NOV12o GATIGASILLIFFFIALVFFLYRRRKGSRKDVTLRKLDIKVETVNREPLTMHSDREDDTA
NOV12p GATIGASILLIFFFIALVFFLYRRRKGSRKDVTLRKLDIKVETVNREPLTMHSDREDDTA
NOV12q GATIGASILLIFFFIALVFFLYRRRKGSRKDVTLRKLDIKVETVNREPLTMHSDREDDTA
NOV12a SVSTATRVMKAIYSSFKDDVDLKQDLRCDTIDTREEYEMKDPTNGYYNVRAHEDRPSSRA
NOV12b SVSTATRVMKAIYSSFKDDVDLKQDLRCDTIDTREEYEMKDPTNGYYNVPAHEDRPSSRA
NOV12c SVSTATRVMKAIYSSFMDDVDLKQDLRCDTIDTREEYEMKDPTNGYYNVRAHEDRPSSRA
NOV12d SVSTATRVMKAIYSSFMDDVDLKQDLRCDTIDTREEYEMKDPTNGYYNVRAHEDRPSSEA
NOV12e SVSTATRVMKAIYSSFKDDVDLKQDLRCDTIDTREEYEMKDPTNGYYNVRAHEDRPSSEA
NOV12f SVSTATRVMKAIYSSFKDDVDLKQDLRCDTIDTREEYEMKDPTNGYYNVRAHEDRPSSRA
NOV12g ------------------------------------------------------------
NOV12h SVSTATRVMKAIYSSFKDDVDLKQDLRCDTIDTREEYEMKDPTNGYYNVRAHEDRPSSRA
NOV12i SVSTATRVMKAIYSSFKDDVDLKQDLRCDTIDTREEYEMKDPTNGYYNVRAHEDRPSSRA
NOV12j ------------------------------------------------------------
NOV12k ------------------------------------------------------------
NOV12l ------------------------------------------------------------
NOV12m ------------------------------------------------------------
NOV12n SVSTATRVMKAIYSSFMDDVDLKQDLRCDTIDTREEYEMKDPTNGYYNVRAHEDRPSSRA
NOV12o SVSTATRVMKAIYSSFKDDVDLKQDLRCDTIDTREEYEMKDPTNGYYNVRAHEDRPSSRA
NOV12p SVSTATRVMKAIYSSFKDDVDLKQDLRCDTIDTREEYEMKDPTNGYYNVRAHEDRPSSRA
NOV12q SVSTATRVMKAIYSSFKDDVDLKQDLRCDTIDTREEYEMKDPTNGYYNVRAHEDRPSSRA
NOV12a VLYADYRAPGPARFDGRPSSRLSHSSGYAQLNTYSRGPASDYGPEPTPPGPAAPAGTDTT
NOV12b VLYADYPAPGPARFDGRPSSRLSHSSGYAQLNTYSRGPASDYGPEPTPPGPAAPAGTDTT
NOV12c VLYADYRAPGPARFDGRPSSRLSHSSGYAQLNTYSRGPASDYGPEPTPPGPAAPAGTDTT
NOV12d VLYADYRAPGPARFDGRPSSRLSHSSGYAQLNTYSRGPASDYGPEPTPPGPAAPAGTDTT
NOV12e VLYADYRAPGPARFDGRPSSRLSHSSGYAQLNTYSRGPASDYQPEPTPPGPAAPAGTDTT
NOV12f VLYADYRAPGPARFDGRPSSRLSHSSGYAQLNTYSRGPASDYGPEPTPPGPAAPAGTDTT
NOV12g ------------------------------------------------------------
NOV12h VLYADYRAPGPARFDGRPSSRLSHSSGYAQLNTYSRGPASDYGPEPTPPGPAAPAGTDTT
NOV12i VLYADYRAPGPARFDGRPSSRLSHSSGYAQLNTYSRGPASDYGPEPTPPGPAAPAGTDTT
NOV12j ------------------------------------------------------------
NOV12k ------------------------------------------------------------
NOV12l ------------------------------------------------------------
NOV12m ------------------------------------------------------------
NOV12n VLYADYRAPGPARFDGRPSSRLSHSSGYAQLNTYSRGPASDYGPEPTPPGPAAPAGTDTT
NOV12o VLYADYRAPGPARFDGRPSSRLSHSSGYAQLNTYSRGPASDYGPEPTPPGPAAPAGTDTT
NOV12p VLYADYRAPGPARFDGRPSSRLSHSSGYAQLNTYSRGPASDYGPEPTPPGPAAPAGTDTT
NOV12q VLYADYRAPGPARFDGRPSSRLSHSSGYAQLNTYSRGPASDYGPEPTPPGPAAPAGTDTT
NOV12a SQLSYENYEKFNSHPFPGAAGYPTYRLGYPQAPPSGLERTPYEAYDPIGKYATATRFSYT
NOV12b SQLSYENYEKFNSHPFPGAAGYPTYRLGYPQAPPSGLERTPYEAYDPIGKYATATRFSYT
NOV12c SQLSYENYEKFNSHPFPGAAGYPTYRLGYPQAPPSGLERTPYEAYDPIGKYATATRFSYT
NOV12d SQLSYENYEKFNSHPFPGAAGYPTYRLGYPQAPPSGLERTPYEAYDPIGKYATATRFSYT
NOV12e SQLSYENYEKFNSHPFPGAAGYPTYRLGYPQAPPSGLERTPYEAYDPIGKYATATRFSYT
NOV12f SQLSYENYEKFNSHPFPGAAGYPTYRLGYPQAPPSGLERTPYEAYDPIGKYATATRFSYT
NOV12g ------------------------------------------------------------
NOV12h SQLSYENYEKFNSHPFPGAAGYPTYRLGYPQAPPSGLERTPYEAYDPIGKYATATRFSYT
NOV12i SQLSYENYEKFNSHPFPGAAGYPTYRLGYPQAPPSGLERTPYEAYDPIGKYATATRFSYT
NOV12j ------------------------------------------------------------
NOV12k ------------------------------------------------------------
NOV12l ------------------------------------------------------------
NOV12m ------------------------------------------------------------
NOV12n SQLSYENYEKFNSHPFPGAAGYPTYRLGYPQAPPSGLERTPYEAYDPIGKYATATRFSYT
NOV12o SQLSYENYEKFNSHPFPGAAGYPTYRLGYPQAPPSGLERTPYEAYDPIGKYATATRFSYT
NOV12p SQLSYENYEKFNSHPFPGAAGYPTYRLGYPQAPPSGLERTPYEAYDPIGKYATATRFSYT
NOV12q SQLSYENYEKFNSHPFPGAAGYPTYRLGYPQAPPSGLERTPYEAYDPIGKYATATRFSYT
NOV12a SQHSDYGQRFQQRMQTHV---
NOV12b SQHSDYGQRFQQRMQTHV---
NOV12c SQHSDYGQRFQQRMQTHV---
NOV12d SQHSDYGQRFQQRMQTHVLE-
NOV12e SQHSDYGQRFQQRMQTHVVDG
NOV12f SQHSDYGQRFQQRMQTHVVD-
NOV12g ---------------------
NOV12h SQHSDYGQRFQQRMQTHV---
NOV12i SQHSDYGQRFQQRMQTHV---
NOV12j ---------------------
NOV12k ---------------------
NOV12l ---------------------
NOV12m ---------------------
NOV12n SQHSDYGQRFQQRMQTHV---
NOV12o SQHSDYGQRFQQRMQTHV---
NOV12p SQHSDYGQRFQQRMQTHV---
NOV12q SQHSDYGQRFQQRMQTHV---
NOV12a (SEQ ID NO: 320)
NOV12b (SEQ ID NO: 322)
NOV12c (SEQ ID NO: 324)
NOV12d (SEQ ID NO: 326)
NOV12e (SEQ ID NO: 328)
NOV12f (SEQ ID NO: 330)
NOV12g (SEQ ID NO: 332)
NOV12h (SEQ ID NO: 334)
NOV12i (SEQ ID NO: 336)
NOV12j (SEQ ID NO: 338)
NOV12k (SEQ ID NO: 340)
NOV12l (SEQ ID NO: 342)
NOV12m (SEQ ID NO: 344)
NOV12n (SEQ ID NO: 346)
NOV12o (SEQ ID NO: 348)
NOV12p (SEQ ID NO: 350)
NOV12q (SEQ ID NO: 352)

Further analysis of the NOV12a protein yielded the following properties shown in Table 12C.

TABLE 12C
Protein Sequence Properties NOV12a

| | |
|---|---|
| SignalP analysis: | Cleavage site between residues 17 and 18 |
| PSORT II analysis: | |

| PSG: | a new signal peptide prediction method | |
|---|---|---|
| | N-region: | length 0; pos. chg 0; neg. chg 0 |
| | H-region: | length 12; peak value 10.45 |
| | PSG score: | 6.05 |

| | |
|---|---|
| GvH: | von Heijne's method for signal seq. recognition |
| | GvH score (threshold: −2.1): 0.73 |
| | possible cleavage site: between 16 and 17 |

>>> Seems to have a cleavable signal peptide (1 to 16)

| ALOM: | Klein et al's method for TM region allocation | | |
|---|---|---|---|
| | Init position for calculation: 17 | | |
| | Tentative number of TMS(s) for the threshold 0.5: 1 | | |
| | Number of TMS(s) for threshold 0.5: 1 | | |
| | INTEGRAL | Likelihood = | Transmembrane 503-519 |
| | | −12.26 | |

| | |
|---|---|
| PERIPHERAL | Likelihood = 3.61 (at 38) |

| | |
|---|---|
| | ALOM score: −12.26 (number of TMSs: 1) |
| MTOP: | Prediction of membrane topology (Hartmann et al.) |
| | Center position for calculation: 8 |
| | Charge difference: −2.0 C(−1.0)-N(1.0) |
| | N >= C: N-terminal side will be inside |

>>> membrane topology: type 1a (cytoplasmic tail 520 to 757)

| MITDISC: | discrimination of mitochondrial targeting seq | | | |
|---|---|---|---|---|
| | R content: | 1 | Hyd Moment(75): | 1.68 |
| | Hyd Moment(95): | 2.60 | G content: | 1 |
| | D/E content: | 2 | S/T content: | 8 |

| | |
|---|---|
| | Score: −5.22 |
| Gavel: | prediction of cleavage sites for mitochondrial preseq |
| | R-2 motif at 32 TRF\|SQ |

NUCDISC: discrimination of nuclear localization signals

| | |
|---|---|
| | pat4: RRRK (5) at 522 |
| | pat7: none |
| | bipartite: none |
| | content of basic residues: 9.6% |
| | NLS Score: −0.16 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 70.6 |
| COIL: | Lupas's algorithm to detect coiled-coil regions |
| | total: 0 residues |

Final Results (k = 9/23):

44.4%: endoplasmic reticulum
22.2%: Golgi
22.2%: extracellular, including cell wall
11.1%: plasma membrane

>> prediction for CG51373-11 is end (k = 9)

A search of the NOV12a protein against the Geneseq database, a proprietary database that contains sequences published in patents and patent publication, yielded several homologous proteins shown in Table 12D.

TABLE 12D
Geneseq Results for NOV12a

| | | NOV12a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB05749 | Human G protein-coupled receptor | 17 . . . 757 | 739/741 (99%) | 0.0 |
| | NOV1a protein SEQ ID NO: 2 - Homo | 101 . . . 841 | 740/741 (99%) | |
| | sapiens, 841 aa. [WO200200691-A2, | | | |
| | 03-JAN-2002] | | | |
| ABP66884 | Human polypeptide SEQ ID NO 605 - | 57 . . . 757 | 701/717 (97%) | 0.0 |
| | Homo sapiens, 749 aa. | 33 . . . 749 | 701/717 (97%) | |
| | [US2002090672-A1, 11-JUL-2002] | | | |
| ABB10297 | Human cDNA SEQ ID NO: 605 - Homo | 57 . . . 757 | 701/717 (97%) | 0.0 |
| | sapiens, 749 aa. [WO200154474-A2, | 33 . . . 749 | 701/717 (97%) | |
| | 02-AUG-2001] | | | |
| ABB05762 | Human G protein-coupled receptor | 62 . . . 757 | 695/696 (99%) | 0.0 |
| | NOV7b protein SEQ ID NO: 26 - Homo | 1 . . . 696 | 695/696 (99%) | |
| | sapiens, 696 aa. [WO200200691-A2, | | | |
| | 03-JAN-2002] | | | |
| ABG65107 | Human albumin fusion protein #1782 - | 62 . . . 757 | 696/712 (97%) | 0.0 |
| | Homo sapiens, 712 aa. [WO200177137- | 1 . . . 712 | 696/712 (97%) | |
| | A1, 18-OCT-2001] | | | |

In a BLAST search of public sequence databases, the NOV12a protein was found to have homology to the proteins shown in the BLASTP data in Table 12E.

TABLE 12E
Public BLASTP Results for NOV12a

| | | NOV12a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAD23338 | Sequence 1 from Patent | 17 . . . 757 | 739/741 (99%) | 0.0 |
| | WO0200691 - Homo sapiens | 101 . . . 841 | 740/741 (99%) | |
| | (Human), 841 aa. | | | |
| AAO91769 | NEPH1 - Mus musculus (Mouse), | 18 . . . 757 | 717/740 (96%) | 0.0 |
| | 789 aa. | 50 . . . 789 | 728/740 (97%) | |
| Q8CJ59 | NEPH1 - Mus musculus (Mouse), | 18 . . . 757 | 714/740 (96%) | 0.0 |
| | 789 aa. | 50 . . . 789 | 726/740 (97%) | |
| CAD23349 | Sequence 25 from Patent | 62 . . . 757 | 695/696 (99%) | 0.0 |
| | WO0200691 - Homo sapiens | 1 . . . 696 | 695/696 (99%) | |
| | (Human), 696 aa. | | | |
| AAP12626 | NEPH1 - Rattus norvegicus (Rat), | 53 . . . 754 | 678/702 (96%) | 0.0 |
| | 702 aa (fragment). | 1 . . . 702 | 688/702 (97%) | |

PFam analysis predicts that the NOV12a protein contains the domains shown in the Table 12F.

TABLE 12F
Domain Analysis of NOV12a

| | | Identities/ | |
|---|---|---|---|
| | | Similarities | |
| Pfam | NOV12a | for the Matched | Expect |
| Domain | Match Region | Region | Value |
| ig | 35 . . . 102 | 12/72 (17%) | 1.3e−05 |
| | | 47/72 (65%) | |
| ig | 136 . . . 202 | 14/69 (20%) | 0.0023 |
| | | 48/69 (70%) | |
| ig | 322 . . . 373 | 21/55 (38%) | 2.2e−09 |
| | | 43/55 (78%) | |

### Example 13

The NOV13 clone was analyzed, and the nucleotide and encoded polypeptide sequences are shown in Table 13A.

TABLE 13A

NOV13 Sequence Analysis

| | | |
|---|---|---|
| NOV13a, CG51622-04 | SEQ ID NO: 369 | 1826 bp |
| DNA Sequence | ORF Start: ATG at 167 | ORF Stop: TGA at 1721 |

AGGACTCCAGCGTGCCCAGTGACATAAACCCACTCTGCACATACCAGTTGCTCTGTCCCAGCTGTGCT
GCGGTGGTGCTGGGACAAAAGAAGCATGAGACGCAGACTCTGTCCCTCAAAGAACTCCCAGTGCAGGA
CAATGTGGTGCCCAAGGAAGACCCAGGGACATGGGAGTGGAGGACAGTCAAAGAAGGGATCCTGGAAG
AGGTCTGGCATCCTGCACTTGCTGCCCTCTGACACCTGGGAAGATGGCCGGCCCGTGGACCTTCACCC
TTCTCTGTGGTTTGCTGGCAGCCACCTTGATCCAAGCCACCCTCAGTCCCACTGCAGTTCTCATCCTC
GGCCCAAAAGTCATCAAAGAAAAGCTGACACAGGAGCTGAAGGACCACAACGCCACCAGCATCCTGCA
GCAGCTGCCGCTGCTCAGTGCCATGCGGGAAAAGCCAGCCGGAGGCATCCCTGTGCTGGGCAGCCTGG
TGAACACCGTCCTGAAGCACATCATCTGGCTGAAGGTCATCACAGCTAACATCCTCCAGCTGCAGGTG
AAGCCCTCGGCCAATGACCAGGAGCTGCTAGTCAAGATCCCCCTGGACATGGTGGCTGGATTCAACAC
GCCCCTGGTCAAGACCATCGTGGAGTTCCACATGACGACTGAGGCCCAAGCCACCATCCGCATGGACA
CCAGTGCAAGTGGCCCCACCCGCCTGGTCCTCAGTGACTGTGCCACCAGCCATGGGAGCCTGCGCATC
CAACTGCTGCATAAGCTCTCCTTCCTGGTGAACGCCTTAGCTAAGCAGGTCATGAACCTCCTAGTGCC
ATCCCTGCCCAATCTAGTGAAAAACCAGCTGTGTCCCGTGATCGAGGCTTCCTTCAATGGCATGTATG
CAGACCTCCTGCAGCTGGTGAAGGTGCCCATTTCCCTCAGCATTGACCGTCTGGAGTTTGACCTTCTG
TATCCTGCCATCAAGGGTGACACCATTCAGCTCTACCTGGGGGCCAAGTTGTTGGACTCACAGGGAAA
GGTGACCAAGTGGTTCAATAACTCTGCAGCTTCCCTGACAATGCCCACCCTGGACAACATCCCGTTCA
GTCCTGTTGGACTCTGTGCTTCCTGAGAGTGCCCATCGGCTGAAGTCAAGCATCGGGCTGATCAATGA
AAAGGCTGCAGATAAGCTGGGATCTACCCAGATCGTGAAGATCCTAACTCAGGACACTCCCGAGTTTT
TTATAGACCAAGGCCATGCCAAGGTGGCCCAACTGATCGTGCTGGAAGTGTTTCCCTCCAGTGAAGCC
CTCCGCCCTTTGTTCACCCTGGGCATCGAAGCCAGCTCGGAAGCTCAGTTTTACACCAAAGGTGACCA
ACTTATACTCAACTTGAATAACATCAGCTCTGATCGGATCCAGCTGATGAACTCTGGGATTGGCTGGT
TCCAATGCCCCTTCTCTCTCTCACAGCCTGATGTTCTGAAAAACATCATCACTGAGATCATCCACTCC
ATCCTGCTGCCGAACCAGAATGGCAAATTAAGATCTGGGGTCCCAGTGTCATTGGTGAAGGCCTTGGG
CCAGCTCTCCTGTCTCCCAGTGAAGACTTGGATGGCAGCCATCAGGGAAGGCTGGGTCCCAGCTGGGA
GTATGGGTGTGAGCTCTATAGACCATCCCTCTCTGCAATCAATAAACACTTGCCTGTG

| | | | |
|---|---|---|---|
| NOV13a, CG51622-04 | | | |
| Protein Sequence | SEQ ID NO: 370 | 518 aa | MW at 56020.9kD |

MGVEDSQRRDPGRGLASCTCCPLTPGKMAGPWTFTLLCGLLAATLIQATLSPTAVLILGPKVIKEKLT
QELKDHNATSILQQLPLLSAMREKPAGGIPVLGSLVNTVLKHIIWLKVITANILQLQVKPSANDQELL
VKIPLDMVAGFNTPLVKTIVEFHMTTEAQATIRMDTSASGPTRLVLSDCATSHGSLRIQLLHKLSFLV
NALAKQVMNLLVPSLPNLVKNQLCPVIEASFNGMYADLLQLVKVPISLSIDRLEFDLLYPAIKGDTIQ
LYLGAKLLDSQGKVTKWFNNSAASLTMPTLDNIPFSLIVSQDVVKAAVAAVLSPEEFMVLLDSVLPES
AHRLKSSIGLINEKAADKLGSTQIVKILTQDTPEFFIDQGHAKVAQLIVLEVFPSSEALRPLFTLGIE
ASSEAQFYTKGDQLILNLNNISSDRIQLMNSGIGWFQCPFSLSQPDVLKNIITEIIHSILLPNQNGKL
RSGVPVSLVKALGFEAAESSLTKDALVLTPASLWKPSSPVSQ

| | | |
|---|---|---|
| NOV13b, CG51622-03 | SEQ ID NO: 371 | 1452 bp |
| DNA Sequence | ORF Start: ATG at 44 | ORF Stop: TGA at 1346 |

AGGTCTGCCATCCTGCACTTGCTGCCCTCTGACACCTGGGAAGATGGCCGGCCCGTGGACCTTCACCC
CTCTCTGTGGTTTGCTGGCAGCCACCTTGATCCAAGCCACCCTCAGTCCCACTGCAGTTCTCATCCTC
GGCCCAAAAGTCATCAAAGAAAAGCTGACACAGGAGCTGAAGGACCACAACGCCACCAGCATCCTGCA
GCAGCTGCCGCTGCTCAGTGCCATGCGGGAAAAGCCAGCCGGAGGCATCCCTGTGCTGGGCAGCCTGG
TGAACACCGTCCTGAAGCACGTCATCTGGCTGAAGGTCATCACAGCTAACATCCTCCAGCTGCAGGTG
AAGCCCTCGGCCAATGACCAGGAGCTGCTAGTCAAGATCCCCCTGGACATGGTGGCTGGATTCAACAC
GCTCTCCTTCCTGGTGAACGCCTTAGCTAAGCAGGTCATGAACCTCCTAGTGCCATCCCTGCCCAATC
TAGTGAAAAACCAGCTGTGTCCCGTGATCGAGGCTTCCTTCAATGGCATGTATGCAGACCTCCTGCAG
CTGGTGAAGGTGCCCATTTCCCTCAGCATTGACCGTCTGGAGTTTGACCTTCTGTATCCTGCCATCAA
GGGTGACACCATTCAGCTCTACCTGGGGGCCAAGTTGTTGGACTCACAGGGAAAGGTGACCAAGTGGT
TCAATAACTCTGCAGCTTCCCTGACAATGCCCACCCTGGACAACATCCCGTTCAGCCTCATCGTGAGT
CAGGACGTGGTGAAAGCTGCAGTGGCTGCTGTGCTCTCTCCAGAAGAATTCATGGTCCTGTTGGACTC
CATGCCAAGGTGGCCCAACTGATCGTGCTGGAAGTGTTTCCCTCCAGTGAAGCCCTCCGCCCTTTGTT
CACCCTGGGCATCGAAGCCAGCTCGGAAGCTCAGTTTTACACCAAAGGTGACCAACTTATACTCAACT
TGAATAACATCAGCTCTGATCGGATCCAGCTGATGAACTCTGGGATTGGCTGGTTCCAACCTGATGTT
CTGAAAAACATCATCACTGAGATCATCCACTCCATCCTGCTGCCGAACCAGAATGGCAAATTAAGATC
TGGGGTCCCAGTGTCATTGGTGAAGGCCTTGGGATTCGAGGCAGCTGAGTCCTCACTGAGCAAGGATG
CCCTTGTGCTTACTCCAGCCTCCTTGTGGAAACCCACCTCTCCTGTCTCCCAGTGAAGACTTGGATGG
CAGCCATCAGGGAAGGCTGGGTCCCAGCTGGGAGTATGGGTGTGAGCTCTATAGACCATCCCTCTCTG
CAATCAATAAACACTTGCCTGTGA

| | | | |
|---|---|---|---|
| NOV13b, CG51622-03 | | | |
| Protein Sequence | SEQ ID NO: 372 | 1434 aa | MW at 46965.5kD |

MAGPWTFTPLCGLLAATLIQATLSPTAVLILGPKVIKEKLTQELKDHNATSILQQLPLLSAMREKPAG
GIPVLGSLVNTVLKHVIWLKVITANILQLQVKPSANDQELLVKIPLDMVAGFNTLSFLVNALAKQVMN
LLVPSLPNLVKNQLCPVIEASFNGMYADLLQLVKVPISLSIDRLEFDLLYPAIKGDTIQLYLGAKLLD
SQGKVTKWFNNSAASLTMPTLDNIPFSLIVSQDVVKAAVAAVLSPEEFMVLLDSVLPESAHRLKSSIG
LINEKAADKLGSTQIVKILTQDTPEFFIDQGHAKVAQLIVLEVFPSSEALRPLFTLGIEASSEAQFYT
KGDQLILNLNNISSDRIQLNNSGIGWFQPDVLKNIITEIIHSILLPNQNGKLRSGVPVSLVKALGFEA
AESSLSKDALVLTPASLWKPTSPVSQ

| | | |
|---|---|---|
| NOV13c, CG51622-05 | SEQ ID NO: 373 | 1580 bp |
| DNA Sequence | ORF Start: ATG at 77 | ORF Stop: TGA at 1475 |

AGAGAGGAGCGGGCCGAGGACTCCAGCGTGCCCAGGTCTGGCATCCTGCACTTGCTGCCCTCTGACAC
CTGGGAAGATGGCCGGCCCGTGGACCTTCACCCTTCTCTGTGGTTTGCTGGCAGCCACCTTGATCCAA
GCCACCCTCAGTCCCACTGCAGTTCTCATCCTCGGCCCAAAAGTCATCAAAGAAAAGCTGACACAGGA
GCTGAAGGACCACAACGCCACCAGCATCCTGCAGCAGCTGCCGCTGCTCAGTGCCATGCGGGAAAAGC
CAGCCGGAGGCATCCCTGTGCTGGGCAGCCTGGTGAACACCGTCCTGAAGCACGTCATCTGGCTGAAG
GTCATCACAGCTAACATCCTCCAGCTGCAGGTGAAGCCCTCGGCCAATGACCAGGAGCTGCTAGTCAA
GATCCCCCTGGACATGGTGGCTGGATTCAACACGCCCCTGGTCAAGACCATCGTGGAGTTCCACATGA
CGACTGAGGCCCAAGCCACCATCCGCATGGACACCAGTGCAAGTGGCCCCACCCGCCTGGTCCTCAGT
GACTGTGCCACCAGCCATGGGAGCCTGCGCATCCAACTGCTGCATAAGCTCTCCTTCCTGGTGAACGC
CTTAGCTAAGCAGGTCATGAACCTCCTAGTGCCATCCCTGCCCAATCTAGTGAAAAACCAGCTGGTGA
AGGTGCCCATTTCCCTCAGCATTGACCGTCTGGAGTTTGACCTTCTGTATCCTGCCATCAAGGGTGAC
ACCATTCAGCTCTACCTGGGGGCCAAGTTGTTGGACTCACAGGGAAAGGTGACCAAGTGGTTCAATAA
CTCTGCAGCTTCCCTGACAATGCCCACCCTGGACAACATCCCGTTCAGCCTCATCGTGAGTCAGGACG
TGGTGAAAGCTGCAGTGGCTGCTGTGCTCTCTCCAGAAGAATTCATGGTCCTGTTGGACTCTGTGCTT
CCTGAGAGTGCCCATCGGCTGAAGTCAAGCATCGGGCTGATCAATGAAAAGGCTGCAGATAAGCTGGG
ATCTACCCAGATCGTGAAGATCCTAACTCAGGACACTCCCGAGTTTTTTATAGACCAAGGCCATGCCA
AGGTGGCCCAACTGATCGTGCTGGAAGTGTTTCCCTCCAGTGAAGCCCTCCGCCCTTTGTTCACCCTG
GGCATCGAAGCCAGCTCGGAAGCTCAGTTTTACACCAAAGGTGACCAACTTATACTCAACTTGAATAA
CATCAGCTCTGATCGGATCCAGCTGATGAACTCTGGGATTGGCTGGTTCCAACCTGATGTTCTGAAAA
ACATCATCACTGAGATCATCCACTCCATCCTGCTGCCGAACCAGAATGGCAAATTAAGATCTGGGGTC
CCAGTGTCATTGGTGAAGGCCTTGGGATTCGAGGCAGCTGAGTCCTCACTGAGCAAGGATGCCCTTGT
GCTTACTCCAGCCTCCTTGTGGAAACCCACCTCTCCTGTCTCCCAGTGAAGACTTGGATGGCAGCCAT
AGGGAAGGCTGGGTCCCAGCTGGGAGTATGGGTGTGAGCTCTATAGACCATCCCTCTCTGCAATCAA
TAAACACTTGCCTGTG

| | | | |
|---|---|---|---|
| NOV13c, CG51622-05 | | | |
| Protein Sequence | SEQ ID NO: 374 | 466 aa | MW at 50460.5kD |

MAGPWTFTLLCGLLAATLIQATLSPTAVLILGPKVIKEKLTQELKDHNATSILQQLPLLSAMREKPAG
GIPVLGSLVNTVLKHVIWLKVITANILQLQVKPSANDQELLVKIPLDMVAGFNTPLVKTIVEFHMTTE
AQATIRMDTSASGPTRLVLSDCATSHGSLRIQLLHKLSFLVNALAKQVMNLLVPSLPNLVKNQLVKVP
ISLSIDRLEFDLLYPAIKGDTIQLYLGAKLLDSQGKVTKWFNNSAASLTMPTLDNIPFSLIVSQDVVK
AAVAAVLSPEEFMVLLDSVLPESAHRLKSSIGLINEKAADKLGSTQIVKILTQDTPEFFIDQGHAKVA
QLIVLEVFPSSEALRPLFTLGIEASSEAQFYTKGDQLILNLNNISSDRIQLMNSGIGWFQPDVLKNII
TEIIHSILLPNQNGKLRSGVPVSLVKALGFEAAESSLSKDALVLTPASLWKPTSPVSQ

| | | |
|---|---|---|
| NOV13d, CG51622-01 | SEQ ID NO: 375 | 963 bp |
| DNA Sequence | ORF Start: ATG at 1 | ORF Stop: TAA at 961 |

ATGGCCGGCCCGTGGACCTTCACCCTTCTCTGTGGTTTGCTGGCAGCCACCTTGATCCAAGCCACCCT
CAGTCCCACTGCAGTTCTCATCCTCGGCCCAAAAGTCATCAAAGAAAGTCTGACACAGGAGCTGAAGG
ACCACAACGCCACCAGCATCCTGCAGCAGCTGCCGCTGCTCAGTGCCATGCGGGAAAAGCCAGCCGGA
GGCATCCCTGTGCTGGGCAGCCTGGTGAACACCGTCCTGAAGCACATCACCCCATCCAGGCTGAAGGT
CATCACAGCTAACATCCTCCAGCTGCAGGTGAAGCCCTCGGCCAATGACCAGGAGCTGCTAGTCAAGA
TCCCCCTGGACATGGTGGCTGGATTCAACACGCCCCTGGTCAAGACCATCGTGGAGTTCCACATGACG
ACTGAGGCCCAAGCCACCATCCGCATGGACACCAGTGCAAGTGGCCCCACCCGCCTGGTCCTCAGTGA
CTGTGCCACCAGCCATGGGAGCCTGCGCATCCAACTGCTGCATAAGCTCTCCTTCCTGGTGAACGCCT
TAGCTAAGCAGGTCATGAACCTCCTAGTGCCATCCCTGCCCAATCTAGTGAAAAACCAGCTGTGTCCC
GTGATCGAGGCTTCCTTCAATGGCATGTATGCAGACCTCCTGCAGCTGGTGAAGGGTAGGTGCTCTGC
TCTCTCTCCCACTTTTTCCTTTACTACGGAGCTGGCCTCCAGACCCGGAAAGGTGACCAAGTGGTTCA
ATAACTCTGCAGCTTCCCTGACAATGCCCACCCTGGACAACATCCCGTTCAGCCTCATCGTGAGTCAG
GACGTGGTGAAAGCTGCAGTGGCTGCTGTGCTCTCTCCAGAAGAATTCATGGTCCTGTTGGACTCTGT
GGTAAACCTCAGCACAAGGCAGAGAATAGGGCCGCCCAGGCCACATCATAGGAATTTCCTGAACACAG
GGTGCCCCTAA

| | | | |
|---|---|---|---|
| Nov13d, CG51622-01 | | | |
| Protein Sequence | SEQ ID NO: 376 | 320 aa | MW at 34533.2kD |

MAGPWTFTLLCGLLAATLIQATLSPTAVLILGPKVIKESLTQELKDHNATSILQQLPLLSAMREKPAG
GIPVLGSLVNTVLKHITPSRLKVITANILQLQVKPSANDQELLVKIPLDMVAGFNTPLVKTIVEFHMT
TEAQATIRMDTSASGPTRLVLSDCATSHGSLRIQLLHKLSFLVNALAKQVMNLLVPSLPNLVKNQLCP
VIEASFNGMYADLLQLVKGRCSALSPTFSFTTELASRPGKVTKWFNNSAASLTMPTLDNIPFSLIVSQ
DVVKAAVAAVLSPEEFMVLLDSVVNLSTRQRIGPPRPHHRNFLNTGCP

| | | |
|---|---|---|
| NOV13e, 275698370 | SEQ ID NO: 377 | 1024 bp |
| DNA Sequence | ORF Start: at 2 | ORF Stop: end of sequence |

CACCGGATCCACCATGGCCGGCCCGTGGACCTTCACCCTTCTCTGTGGTTTGCTGGCAGCCACCTTGA
TCCAAGCCACCCTCAGTCCCACTGCAGTTCTCATCCTCGGCCCAAAAGTCATCAAAGAAAAGCTGACA
CAGGAGCTGAAGGACCACAACGCCACCAGCATCCTGCAGCAGCTGCCGCTGCTCAGTGCCATGCGGGA
AAAGCCAGCCGGAGGCATCCCTGTGCTGGGCAGCCTGGTGAACACCGTCCTGAAGCACGTCATCTGGC
TGAAGGTCATCACAGCTAACATCCTCCAGCTGCAGGTGAAGCCCTCGGCCAATGACCAGGAGCTGCTA
GTCAAGATCCCCCTGGACATGGTGGCTGGATTCAACACGCCCCTGGTCAAGACCATCGTGGAGTTCCA
CATGACGACTGAGGCCCAAGCCACCATCCGCATGGACACCAGTGCAAGTGGCCCCACCCGCCTGGTCC
TCAGTGACTGTGCCACCAGCCATGGGAGCCTGCGCATCCAACTGCTGCATAAGCTCTCCTTCCTGGTG
GTGTCCCGTGATCGAGGCTTCCTTCAATGGCATGTATGCAGACCTCCTGCAGCTGGTGAAGGTGCCCA
TTTCCCTCAGCATTGACCGTCTGGAGTTTGACCTTCTGTATCCTGCCATCAAGGGTGACACCATTCAG
TTCCCTGACAATGCCCACCCTGGACAACATCCCGTTCAGCCTCATCGTGAGTCAGGACGTGGTGAAAG
CTGCAGTGGCTGCTGTGCTCTCTCCAGAAGAATTCATGGTCCTGTTGGACTCTGTGGTAAACCTCAGC
ACAAGGCAGAGAATAGGGCCGCCCAGGCCACATCATAGGAATTTCCTGAACACAGGGTGCCCCCTCGA
GGGC

| | | | |
|---|---|---|---|
| NOV13e, 275698370 | | | |
| Protein Sequence | SEQ ID NO: 378 | 341 aa | MW at 36955.1kD |

TGSTMAGPWTFTLLCGLLAATLIQATLSPTAVLILGPKVIKEKLTQELKDHNATSILQQLPLLSAMRE
KPAGGIPVLGSLVNTVLKHVIWLKVITANILQLQVKPSANDQELLVKIPLDMVAGFNTPLVKTIVEFH
MTTEAQATIRMDTSASGPTRLVLSDCATSHGSLRIQLLHKLSFLVNALAKQVMNLLVPSLPNLVKNQL
CPVIEASFNGMYADLLQLVKVPISLSIDRLEFDLLYPAIKGDTIQLYLGAKLLDSQGKVTKWFNNSAA
SLTMPTLDNIPFSLIVSQDVVKAAVAAVLSPEEFMVLLDSVVNLSTRQRIGPPRPHHRNFLNTGCPLE
IG

| | | |
|---|---|---|
| NOV13f, 274054187 | SEQ ID NO: 379 | 946 bp |
| DNA Sequence | ORF Start: at 2 | ORF Stop: end of sequence |

CACCGGATCCCCCACTGCAGTTCTCATCCTCGGCCCAAAAGTCATCAAAGAAAAGCTGACACAGGAGC
GCCGGAGGCATCCCTGTGCTGGGCAGCCTGGTGAACACCGTCCTGAAGCACGTCATCTGGCTGAAGGT
TCCCCCTGGACATGGTGGCTGGATTCAACACGCCCCTGGTCAAGACCATCGTGGAGTTCCACATGACG
ACTGAGGCCCAAGCCACCATCCGCATGGACACCAGTGCAAGTGGCCCCACCCGCCTGGTCCTCAGTGA
CTGTGCCACCAGCCATGGGAGCCTGCGCATCCAACTGCTGCATAAGCTCTCCTTCCTGGTGAACGCCT
TAGCTAAGCAGGTCATGAACCTCCTAGTGCCATCCCTGCCCAATCTAGTGAAAAACCAGCTGTGTCCC
GTGATCGAGGCTTCCTTCAATGGCATGTATGCAGACCTCCTGCAGCTGGTGAAGGTGCCCATTTCCCT
CAGCATTGACCGTCTGGAGTTTGACCTTCTGTATCCTGCCATCAAGGGTGACACCATTCAGCTCTACC
TGGGGGCCAAGTTGTTGGACTCACAGGGAAAGGTGACCAAGTGGTTCAATAACTCTGCAGCTTCCCTG
ACAATGCCCACCCTGGACAACATCCCGTTCAGCCTCATCGTGAGTCAGGACGTGGTGAAAGCTGCAGT
GGCTGCTGTGCTCTCTCCAGAAGAATTCATGGTCCTGTTGGACTCTGTGGTAAACCTCAGCACAAGGC
AGAGAATAGGGCCGCCCAGGCCACATCATAGGAATTTCCTGAACACAGGGTGCCCCCTCGAG

| | | | |
|---|---|---|---|
| NOV13f, 274054187 | | | |
| Protein Sequence | SEQ ID NO: 380 | 315 aa | MW at 34322.0kD |

TGSPTAVLILGPKVIKEKLTQELKDHNATSILQQLPLLSAMREKPAGGIPVLGSLVNTVLKHVIWLKV
ITANILQLQVKPSANDQELLVKIPLDMVAGFNTPLVKTIVEFHMTTEAQATIRMDTSASGPTRLVLSD
CATSHGSLRIQLLHKLSFLVNALAKQVMNLLVPSLPNLVKNQLCPVIEASFNGMYADLLQLVKVPISL
SIDRLEFDLLYPAIKGDTIQLYLGAKLLDSQGKVTKWFNNSAASLTMPTLDNIPFSLIVSQDVVKAAV
AAVLSPEEFMVLLDSVVNLSTRQRIGPPRPHHRNFLNTGCPLE

| | | |
|---|---|---|
| NOV13g, CG51622-02 | SEQ ID NO: 381 | 1035 bp |
| DNA Sequence | ORF Start: ATG at 79 | ORF Stop: TAA at 1033 |

AGAGCGTTGGGTCACGTGAGGACTCCAGCGTGCCCAGGTCTGGCATCCTGCACTTACTGCCCTCTGAC
ACCTGGGAAGATGGCCGGCCCGTGGACCTTCACCCTTCTCTGTGGTTTGCTGGCAGCCACCTTGATCC
AAGCCACCCTCAGTCCCACTGCAGTTCTCATCCTCGGCCCAAAAGTCATCAAAGAAAAGCTGACACAG
GAGCTGAAGGACCACAACGCCACCAGCATCCTGCAGCAGCTGCCGCTGCTCAGTGCCATGCGGGAAAA
GCCAGCCGGAGGCATCCCTGTGCTGGGCAGCCTGGTGAACACCGTCCTGAAGCACATCATCTGGCTGA
AGGTCATCACAGCTAACATCCTCCAGCTGCAGGTGAAGCCCTCGGCCAATGACCAGGAGCTGCTAGTC
AAGATCCCCCTGGACATGGTGGCTGGATTCAACACGCCCCTGGTCAAGACCATCGTGGAGTTCCACAT
GACGACTGAGGCCCAAGCCACCATCCGCATGGACACCAGTGCAAGTGGCCCCACCCGCCTGGTCCTCA
GTGACTGTGCCACCAGCCATGGGAGCCTGCGCATCCAACTGCTGCATAAGCTCTCCTTCCTGGTGAAC
GCCTTAGCTAAGCAGGTCATGAACCTCCTAGTGCCATCCCTGCCCAATCTAGTGAAAAACCAGCTGTG
TCCCGTGATCGAGGCTTCCTTCAATGGCATGTATGCAGACCTCCTGCAGCTGGTGAAGGGTAGGTGCT
CTGCTCTCTCTCCCACTTTTTCCTTTACTACGGAGCTGGCCTCCAGACCCGGAAAGGTGACCAAGTGG
TTCAATAACTCTGCAGCTTCCCTGACAATGCCCACCCTGGACAACATCCCGTTCAGCCTCATCGTGAG
TCAGGACGTGGTGAAAGCTGCAGTGGCTGCTGTGCTCTCTCCAGAAGAATTCATGGTCCTGTTGGACT
CTGTGGTAAACCTCAGCACAAGGCAGAGAATAGGGCCGCCCAGGCCACATCATAGGAATTTCCTGAAC
ACAGGGTGCCCCTAA

| | | | |
|---|---|---|---|
| NOV13g, CG51622-02 | | | |
| Protein Sequence | SEQ ID NO: 382 | 318 aa | MW at 34432.2kD |

MAGPWTFTLLCGLLAATLIQATLSPTAVLILGPKVIKEKLTQELKDHNATSILQQLPLLSAMREKPAG
GIPVLGSLVNTVLKHIIWLKVITANILQLQVKPSANDQELLVKIPLDMVAGFNTPLVKTIVEFHMTTE
AQATIRMDTSASGPTRLVLSDCATSHGSLRIQLLHKLSFLVNALAKQVMNLLVPSLPNLVKNQLCPVI
EASFNGMYADLLQLVKGRCSALSPTFSFTTELASRPGKVTKWFNNSAASLTMPTLDNIPFSLIVSQDV
IVKAAVAAVLSPEEFMVLLDSVVNLSTRQRIGPPRPHHRNFLNTGCP

| | | |
|---|---|---|
| NOV13h, CG51622-06 | SEQ ID NO: 383 | 1572 bp |
| DNA Sequence | ORF Start: ATG at 124 | ORF Stop: TGA at 1441 |

GGTGTGCAGGATATAAGGTTGGACTTCCAGACCCACTGCCCGGGAGAGGAGAGGAGCGGGCCGAGGAC
TCCAGCGTGCCCAGGTCTGGCATCCTGCACTTGCTGCCCTCTGACACCTGGGAAGATGGCCGGCCCGT
GGACCTTCACCCTTCTCTGTGGTTTGCTGGCAGCCACCTTGATCCAAGCCACCCTCAGTCCCACTGCA
GTTCTCATCCTCGGCCCAAAAGTCATCAAAGAAAAGCTGACACAGGAGCTGAAGGACCACAACGCCAC
CAGCATCCTGCAGCAGCTGCCGCTGCTCAGTGCCATGCGGGAAAAGCCAGCCGGAGGCATCCCTGTGC
TGGGCAGCCTGGTGAACACCGTCCTGAAGCACATCATCTGGCTGAAGGTCATCACAGCTAACATCCTC
CAGCTGCAGGTGAAGCCCTCGGCCAATGACCAGGAGCTGCTAGTCAAGATCCCCCTGGACATGGTGGC
TGGATTCAACACGCCCCTGGTCAAGACCATCGTGGAGTTCCACATGACGACTGAGGCCCTTGCCACCA
TCCGCATGGACACCAGTGCAAGTGGCCCCACCCGCCTGGTCCTCAGTGACTGTGCCACCAGCCATGGG
AGCCTGCGCATCCAACTGCTGCATAAGCTCTCCTTCCTGGTGAACGCCTTAGCTAAGCAGGTCATGAA
CCTCCTAGTGCCATCCCTGCCCAATCTAGTGAAAAACCAGCTGTGTCCCGTGATCGAGGCTTCCTTCA
ATGGCATGTATGCAGACCTCCTGCAGCTGGTGAAGGTGCCCATTTCCCTCAGCATTGACCGTCTGGAG
TTTGACCTTCTGTATCCTGCCATCAAGGGTGACACCATTCAGCTCTACCTGGGGGCCAAGTTGTTGGA
CTCACAGGGAAAGGTGACCAAGTGGTTCAATAACTCTGCAGCTTCCCTGACAATGCCCACCCTGGACA
ACATCCCGTTCAGCCTCATCGTGAGTCACCCGTTCAGCCTCATCGTGAGTCAGGACGTGGTGAAAGCT
GCAGTGGCTGCTGTGCTCTCTCCAGAAGAATTCATGGTCCTGTTGGACTCTGTGCTTCCTGAGAGTGC
CCATCGGCTGAAGTCAAGCATCGGGCTGATCAATGAAAAGGAAGCCAGCTCGGAAGCTCAGTTTTACA
CCAAAGGTGACCAACTTATACTCAACTTGAATAACATCAGCTCTGATCGGATCCAGCTGATGAACTCT
GGGATTGGCTGGTTCCAACCTGATGTTCTGAAAAACATCATCACTGAGATCATCCACTCCATCCTGCT
CAGCTGAGTCCTCACTGACCAAGGATGCCCTTGTGCTTACTCCAGCCTCCTTGTGGAAACCCAGCTCT
CCTGTCTCCCAGTGAAGACTTGGATGGCAGCCATCAGGGAAGGCTGGGTCCCAGTTGGGAGTATGGGT
GTGAGCTCTATAGACCATCCCTCTCTGCAATCAATAAACACTTGCCTGTGAAAAAAAAAAAAAAATAA
AAAAAAAA

| | | | |
|---|---|---|---|
| NOV13h, CG51622-06 | | | |
| Protein Sequence | SEQ ID NO: 384 | 439 aa | MW at 47572.2kD |

MAGPWTFTLLCGLLAATLIQATLSPTAVLILGPKVIKEKLTQELKDHNATSILQQLPLLSAMREKPAG
GIPVLGSLVNTVLKHIIWLKVITANILQLQVKPSANDQELLVKIPLDMVAGFNTPLVKTIVEFHMTTE
AQATIRMDTSASGPTRLXTSDCATSHGSLRIQLLHKLSFLVNALAKQVMNLLVPSLPNLVKNQLCPVI
EASFNGMYADLLQLVKVPISLSIDRLEFDLLYPAIKGDTIQLYLGAKLLDSQGKVTKWFNNSAASLTM
PTLDNIPFSLIVSHPFSLIVSQDVVKAAVAAVLSPEEFMVLLDSVLPESAHRLKSSIGLINEKEASSE
LGFEAAESSLTKDALVLTPASLWKPSSPVSQ

| | | |
|---|---|---|
| NOV13i, CG51622-07 | SEQ ID NO: 385 | 1023 bp |
| DNA Sequence | ORF Start: ATG at 19 | ORF Stop: TAA at 1021 |

CCTCTGACACCTGGGAAGATGGCCGGCCCGTGGACCTTCACCCTTCTCTGTGGTTTGCTGGCAGCCAC
CTTGATCCAAGCCACCCTCAGTCCCACTGCAGTTCTCATCCTCGGCCCAAAAGTCATCAAAGAAAAGC
TGACACAGGAGCTGAAGGACCACAACGCCACCAGCATCCTGCAGCAGCTGCCGCTGCTCAGTGCCATG
CGGGAAAAGCCAGCCGGAGGCATCCCTGTGCTGGGCAGCCTGGTGAACACCGTCCTGAAGCACGTCAT
CTGGCTGAAGGTCATCACAGCTAACATCCTCCAGCTGCAGGTGAAGCCCTCGGCCAATGACCAGGAGC
TGCTAGTCAAGATCCCCCTGGACATGGTGGCTGGATTCAACACGCCCCTGGTCAAGACCATCGTGGAG
TTCCACATGACGACTGAGGCCCAAGCCACCATCCGCATGGACACCAGTGCAAGTGGCCCCACCCGCCT
GGTCCTCAGTGACTGTGCCACCAGCCATGGGAGCCTGCGCATCCAACTGCTGCATAAGCTCTCCTTCC
TGGTGAACGCCTTAGCTAAGCAGGTCATGAACCTCCTAGTGCCATCCCTGCCCAATCTAGTGAAAAAC
GCCCATTTCCCTCAGCATTGACCGTCTGGAGTTTGACCTTCTGTATCCTGCCATCAAGGGTGACACCA
TTCAGCTCTACCTGGGGGCCAAGTTGTTGGACTCACAGGGAAAGGTGACCAAGTGGTTCAATAACTCT
GCAGCTTCCCTGACAATGCCCACCCTGGACAACATCCCGTTCAGCCTCATCGTGAGTCAGGACGTGGT
GAAAGCTGCAGTGGCTGCTGTGCTCTCTCCAGAAGAATTCATGGTCCTGTTGGACTCTGTGGTAAACC
TCAGCACAAGGCAGAGAATAGGGCCGCCCAGGCCACATCATAGGAATTTCCTGAACACAGGGTGCCCC
TAA

| | | | |
|---|---|---|---|
| NOV13l, CG51622-07 | | | |
| Protein Sequence | SEQ ID NO: 386 | 334 aa | MW at 36309.5kD |

MAGPWTFTLLCGLLAATLIQATLSPTAVLILGPKVIKEKLTQELKDHNATSILQQLPLLSAMREKPAG
GIPVLGSLVNTVLKHVIWLKVITANILQLQVKPSANDQELLVKIPLDMVAGFNTPLVKTIVEFHMTTE
AQATIRMDTSASGPTRLVLSDCATSHGSLRIQLLHKLSFLVNALAKQVMNLLVPSLPNLVKNQLCPVI
EASFNGMYADLLQLVKVPISLSIDRLEFDLLYPAIKGDTIQLYLGAKLLDSQGKVTKWFNNSAASLTM
PTLDNIPFSLIVSQDVVKAAVAAVLSPEEFMVLLDSVVNLSTRQRIGPPRPHHRNFLNTGCP

| | | |
|---|---|---|
| NOV13j, CG51622-08 | SEQ ID NO: 387 | 1509 bp |
| DNA Sequence | ORF Start: ATG at 31 | ORF Stop: TGA at 1480 |

TGCACTTGCTGCCCTCTGACACCTGGGAAGATGGCCGGCCCGTGGACCTTCACCCTTCTCTGTGGTTT
GCTGGCAGCCACCTTGATCCAAGCCACCCTCAGTCCCACTGCAGTTCTCATCCTCGGCCCAAAAGTCA
TCAAAGAAAAGCTGACACAGGAGCTGAAGGACCACAACGCCACCAGCATCCTGCAGCAGCTGCCGCTG
CTCAGTGCCATGCGGGAAAAGCCAGCCGGAGGCATCCCTGTGCTGGGCAGCCTGGTGAACACCGTCCT
GAAGCACATCATCTGGCTGAAGGTCATCACAGCTAACATCCTCCAGCTGCAGGTGAAGCCCTCGGCCA
ATGACCAGGAGCTGCTAGTCAAGATCCCCCTGGACATGGTGGCTGGATTCAACACGCCCCTGGTCAAG
ACCATCGTGGAGTTCCACATGACGACTGAGGCCCAAGCCACCATCCGCATGGACACCAGTGCAAGTGG
CCCCACCCGCCTGGTCCTCAGTGACTGTGCCACCAGCCATGGGAGCCTGCGCATCCAACTGCTGCATA
AGCTCTCCTTCCTGGTGAACGCCTTAGCTAAGCAGGTCATGAACCTCCTAGTGCCATCCCTGCCCAAT
CTAGTGAAAAACCAGCTGTGTCCCGTGATCGAGGCTTCCTTCAATGGCATGTATGCAGACCTCCTGCA
GCTGGTGAAGGTGCCCATTTCCCTCAGCATTGACCGTCTGGAGTTTGACCTTCTGTATCCTGCCATCA
AGGGTGACACCATTCAGCTCTACCTGGGGGCCAAGTTGTTGGACTCACAGGGAAAGGTGACCAAGTGG
TTCAATAACTCTGCAGCTTCCCTGACAATGCCCACCCTGGACAACATCCCGTTCAGCCTCGTGAGTCA
GGACGTGGTGAAAGCTGCAGTGGCTGCTGTGCTCTCTCCAGAAGAATTCATGGTCCTGTTGGACTCTG
TGCTTCCTGAGAGTGCCCATCGGCTGAAGTCAAGCATCGGGCTGATCAATGAAAAGGCTGCAGATAAG
CTGGGATCTACCCAGATCGTGAAGATCCTAACTCAGGACACTCCCGAGTTTTTTATAGACCAAGGCCA
TGCCAAGGTGGCCCAACTGATCGTGCTGGAAGTGTTTCCCTCCAGTGAAGCCCTCCGCCCTTTGTTCA
AATAACATCAGCTCTGATCGGATCCAGCTGATGAACTCTGGGATTGGCTGGTTCCAACCTGATGTTCT
GAAAAACATCATCACTGAGATCATCCACTCCATCCTGCTGCCGAACCAGAATGGCAAATTAAGATCTG
GGGTCCCAGTGTCATTGGTGAAGGCCTTGGGATTCGAGGCAGCTGAGTCCTCACTGACCAAGGATGCC
CTTGTGCTTACTCCAGCCTCCTTGTGGAAACCCAGCTCTCCTGTCTCCCAGTGAAGACTTGGATGGCA
GCCATCAGGGAAG

| | | | |
|---|---|---|---|
| NOV13j, CG51622-08 | | | |
| Protein Sequence | SEQ ID NO: 388 | 1483 aa | MW at 52327.7kD |

MAGPWTFTLLCGLLAATLIQATLSPTAVLILGPKVTKEKLTQELKDHNATSILQQLPLLSAMREKPAG
GIPVLGSLVNTVLKHIIWLKVITANILQLQVKPSANDQELLVKIPLDMVAGFNTPLVKTIVEFHMTTE
AQATIRMDTSASGPTRLVLSDCATSHGSLRIQLLHKLSFLVNALAKQVMNLLVPSLPNLVKNQLCPVI
EASFNGMYADLLQLVKVPISLSIDRLEFDLLYPAIKGDTIQLYLGAKLLDSQGKVTKWFNNSAASLTM
PTLDNIPFSLVSQDVVKAAVAAVLSPEEFMVLLDSVLPESAHRLKSSIGLINEKAADKLGSTQIVKIL
TQDTPEFFIDQGHAKVAQLIVLEVFPSSEALRPLFTLGIEASSEAQFYTKGDQLILNLNNISSDRIQL
MNSGIGWFQPDVLKNIITEIIHSILLPNQNGKLRSGVPVSLVKALGFEAAESSLTKDALVLTPASLWK
PSSPVSQ

| | | |
|---|---|---|
| NOV13k, CG51622-09 | SEQ ID NO: 389 | 1392 bp |
| DNA Sequence | ORF Start: at 7 | ORF Stop: at 1387 |

AAGCTTCCCACTGCAGTTCTCATCCTCGGCCCAAAAGTCATCAAAGAAAAGCTGACACAGGAGCTGAA
GGACCACAACGCCACCAGCATCCTGCAGCAGCTGCCGCTGCTCAGTGCCATGCGGGAAAAGCCAGCCG
GAGGCATCCCTGTGCTGGGCAGCCTGGTGAACACCGTCCTGAAACACATCATCTGGCTGATGGTCATC
ACAGCTAACATCCTCCAGCTGCAGGTGAAGCCCTCGGCCAATGACCAGGAGCTGCTAGTCAAGATCCC
CCTGGACATGGTGGCTGGATTCAACACGCCCCTGGTCAAGACCATCGTGGAGTTCCACATGACGACTG
AGGCCCAAGCCACCATCCGCATGGACACCAGTGCAAGTGGCCCCACCCGCCTGGTCCTCAGTGACTGT
GCCACCAGCCATGGGAGCCTGCGCATCCAACTGCTGCATAAGCTCTCCTTCCTGGTGAACGCCTTAGC
TAAGCAGGTCATGAACCTCCTAGTGCCGTCCCTGCCCAATCTAGTGAAAAACCAGCTGTGTCCCGTGA
TCGAGGCTTCCTTCAATGGCATGTATGCAGACCTCCTGCAGCTGGTGAAGGTGCCCATTTCCCTCAGC
ATTGACCGTCTGGAGTTTGACCTTCTGTATCCTGCCATCAAGGGTGACACCATTCAGCTCTACCTGGG
GGCCAAGTTGTTGGACTCACAGGGAAAGGTGACCAAGTGGTTCAATAACTCTGCAGCTTCCCTGACAA
TGCCCACCCTGGACAACATCCCGTTCAGCCTCATCGTGAGTCAGGACGTGGTGAAAGCTGCAGTGGCT
GCTGTGCTCTCTCCAGAAGAATTCATGGTCCTGTTGGACTCTGTGCTTCCTGAGAGTGCCCATCGGCT
GAAGTCAAGCATCGGGCTGATCAATGAAAAGGCTGCAGATAAGCTGGGACCTACCCAGATCGTGAAGA
TCCTAACTCAGGACACTCCCGAGTTTTTTATAGACCAAGGCCATGCCAAGGTGGCCCAACTGATCGTG
CTGGAAGTGTTTCCCTCCAGTGAAGCCCTCCGCCCTTTGTTCACCCTGGGCATCGAAGCCAGCTCGGA
AGCTCAGTTTTACACCAAAGGTGACCAACTTATACTCAACTTGAATAACATCAGCTCTGATCGGATCC
AGCTGATGAACTCTGGGATTGGCTGGTTCCAACCTGATGTTCTGAAAAACATCATCACTGAGATCATC
CACTCCATCCTGCTGCCGAACCAGAATGGCAAATTAAGATCTGGGGTCCCAGTGTCATTGGTGAAGGC
CTTGGGATTCGAGGCAGCTGAGTCCTCACTGACCAAGGATGCCCTTGTGCTTACTCCAGCCTCCTTGT
GGAAACCCAGCTCTCCTGTCTCCCAGCTCGAG

| | | | |
|---|---|---|---|
| NOV13k, CG51622-09 | | | |
| Protein Sequence | SEQ ID NO: 390 | 460 aa | MW at 49975.9kD |

PTAVLILGPKVIKEKLTQELKDHNATSILQQLPLLSANREKPAGGIPVLGSLVNTVLKHIIWLKVITA
NILQLQVKPSANDQELLVKIPLDMVAGFNTPLVKTIVEFHMTTEAQATIRMDTSASGPTRLVLSDCAT
SHGSLRIQLLHKLSFLVNALAKQVMNLLVPSLPNLVKNQLCPVIEASFNGMYADLLQLVKVPISLSID
RLEFDLLYPAIKGDTIQLYLGAKLLDSQGKVTKWFNNSAASLTMPTLDNIPFSLIVSQDVVKAAVAAV
LSPEEFMVLLDSVLPESAHRLKSSIGLINEKAADKLGPTQIVKILTQDTPEFFIDQGHAKVAQLIVLE
VFPSSEALRPLFTLGIEASSEAQFYTKGDQLILNLNNISSDRIQLMNSGIGWFQPDVLKNIITEIIHS
ILLPNQNGKLRSGVPVSLVKALGFEAAESSLTKDALVLTPASLWKPSSPVSQ

| | | |
|---|---|---|
| NOV13l, CG51622-10 | SEQ ID NO: 391 | 1684 bp |
| DNA Sequence | ORF Start: ATG at 77 | ORF Stop: TGA at 1529 |
| AGAGAGGAGCGGGCCGAGGACTCCAGCGTGCCCAGGTCTGGCATCCTGCACTTGCTGCCCTCTGACAC | | |
| CTGGGAAGATGGCCGGCCCGTGGACCTTCACCCTTCTCTGTGGTTTGCTGGCAGCCACCTTGATCCAA | | |
| GCCACCCTCAGTCCCACTGCAGTTCTCATCCTCGGCCCAAAAGTCATCAAAGAAAAGCTGACACAGGA | | |
| GCTGAAGGACCACAACGCCACCAGCATCCTGCAGCAGCTGCCGCTGCTCAGTGCCATGCGGGAAAAGC | | |
| CAGCCGGAGGCATCCCTGTGCTGGGCAGCCTGGTGAACACCGTCCTGAAGCACGTCATCTGGCTGAAG | | |
| GTCATCACAGCTAACATCCTCCAGCTGCAGGTGAAGCCCTCGGCCAATGACCAGGAGCTGCTAGTCAA | | |
| GATCCCCCTGGACATGGTGGCTGGATTCAACACGCCCCTGGTCAAGACCATCGTGGAGTTCCACATGA | | |
| CGACTGAGGCCCAAGCCACCATCCGCATGGACACCAGTGCAAGTGGCCCCACCCGCCTGGTCCTCAGT | | |
| GACTGTGCCACCAGCCATGGGAGCCTGCGCATCCAACTGCTGCATAAGCTCTCCTTCCTGGTGAACGC | | |
| CTTAGCTAAGCAGGTCATGAACCTCCTAGTGCCATCCCTGCCCAATCTAGTGAAAAACCAGCTGTGTC | | |
| CGTGATCTGAGGCTTCCTTCAATGGCATGTATGCAGACCTCCTGCAGCTGGTGAAGGTGCCCATTTCC | | |
| TCAGCATTGAACCGTCTGGAGTTTGACCTTCTGTATCCTGCCATCAAGGGTGACACCATTCAGCTCTA | | |
| CCTGGGGGCCAAGTTGTTGGACTCACAGGGAAAGGTGACCAAGTGGTTCAATAACTCTGCAGCTTCCC | | |
| TGACAATGCCCACCCTGGACAACATCCCGTTCAGCCTCATCGTGAGTCAGGACGTGGTGAAAGCTGCA | | |
| GTGGCTGCTGTGCTCTCTCCAGAAGAATTCATGGTCCTGTTGGACTCTGTGCTTCCTGAGAGTGCCCA | | |
| TCGGCTGAAGTCAAGCATCGGGCTGATCAATGAAAAGGCTGCAGATAAGCTGGGATCTACCCAGATCG | | |
| TGAAGATCCTAACTCAGGACACTCCCGAGTTTTTTATAGACCAAGGCCATGCCAAGGTGGCCCAACTG | | |
| ATCGTGCTGGAAGTGTTTCCCTCCAGTGAAGCCCTCCGCCCTTTGTTCACCCTGGGCATCGAAGCCAG | | |
| CTCGGAAGCTCAGTTTTACACCAAAGGTGACCAACTTATACTCAACTTGAATAACATCAGCTCTGATC | | |
| GGATCCAGCTGATGAACTCTGGGATTGGCTGGTTCCAACCTGATGTTCTGAAAAACATCATCACTGAG | | |
| ATCATCCACTCCATCCTGCTGCCGAACCAGAATGGCAAATTAAGATCTGGGGTCCCAGTGTCATTGGT | | |
| GAAGGCCTTGGGATTCGAGGCAGCTGAGTCCTCACTGAGCAAGGATGCCCTTGTGCTTACTCCAGCCT | | |
| CCTTGTGGAAACCCACCTCTCCTGTCTCCCAGTGAAGACTTGGATGGCAGCCATCAGGGAAGGCTGGG | | |
| TCCCAGCTGGGAGTATGGGTGTGAGCTCTATAGACCATCCCTCTCTGCAATCAATAAACACTTGCCTG | | |
| TGAAAAAAAAAAAAAAAAAAAAAAAAAAAAAAAAAAAAAAAAAAAAAAAAAA | | |

| | | | |
|---|---|---|---|
| NOV13l, CG51622-10 | | | |
| Protein Sequence | SEQ ID NO: 392 | 484 aa | MW at 52426.8kD |

MAGPWTFTLLCGLLAATLIQATLSPTAVLILGPKVIKEKLTQELKDHNATSILQQLPLLSAMREKPAG
GIPVLGSLVNTVLKHVIWLKVITANILQLQVKPSANDQELLVKIPLDMVAGFNTPLVKTIVEFHMTTE
AQATIRMDTSASGPTRLVLSDCATSHGSLRIQLLHKLSFLVNALAKQVMNLLVPSLPNLVKNQLCPVI
EASFNGMYADLLQLVKVPISLSIDRLEFDLLYPAIKGDTIQLYLGAKLLDSQGKVTKWFNNSAASLTM
PTLDNIPFSLIVSQDVVKAAVAAVLSPEEFMVLLDSVLPESAHRLKSSIGLINEKAADKLGSTQIVKI
LTQDTPEFFIDQGHAKVAQLIVLEVFPSSEALRPLFTLGIEASSEAQFYTKGDQLILNLNNISSDRIQ
LMNSGIGWFQPDVLLNIITEIIHSILLPNQNGKLRSGVPVSLVKALGFEAAESSLSKDALVLTPASLW
KPTSPVSQ

| | | |
|---|---|---|
| NOV13m, | | |
| SNP13375774 of | SEQ ID NO: 393 | 963 bp |
| CG51622-01, | ORF Start: ATG at 1 | ORF Stop: TAA at 961 |
| DNA Sequence | SNP Pos: 248 | SNP Change: A to G |

ATGGCCGGCCCGTGGACCTTCACCCTTCTCTGTGGTTTGCTGGCAGCCACCTTGATCCAAGCCACCCT
CAGTCCCACTGCAGTTCTCATCCTCGGCCCAAAAGTCATCAAAGAAAGTCTGACACAGGAGCTGAAGG
ACCACAACGCCACCAGCATCCTGCAGCAGCTGCCGCTGCTCAGTGCCATGCGGGAAAAGCCAGCCGGA
GGCATCCCTGTGCTGGGCAGCCTGGTGAACACCGTCCTGAAGCGCATCACCCCATCCAGGCTGAAGGT
CATCACAGCTAACATCCTCCAGCTGCAGGTGAAGCCCTCGGCCAATGACCAGGAGCTGCTAGTCAAGA
TCCCCCTGGACATGGTGGCTGGATTCAACACGCCCCTGGTCAAGACCATCGTGGAGTTCCACATGACG
ACTGAGGCCCAAGCCACCATCCGCATGGACACCAGTGCAAGTGGCCCCACCCGCCTGGTCCTCAGTGA
CTGTGCCACCAGCCATGGGAGCCTGCGCATCCAACTGCTGCATAAGCTCTCCTTCCTGGTGAACGCCT
TAGCTAAGCAGGTCATGAACCTCCTAGTGCCATCCCTGCCCAATCTAGTGAAAAACCAGCTGTGTCCC
GTGATCGAGGCTTCCTTCAATGGCATGTATGCAGACCTCCTGCAGCTGGTGAAGGGTAGGTGCTCTGC
TCTCTCTCCCACTTTTTCCTTTACTACGGAGCTGGCCTCCAGACCCGGAAAGGTGACCAAGTGGTTCA
ATAACTCTGCAGCTTCCCTGACAATGCCCACCCTGGACAACATCCCGTTCAGCCTCATCGTGAGTCAG
GGTAAACCTCAGCACAAGGCAGAGAATAGGGCCGCCCAGGCCACATCATAGGAATTTCCTGAACACAG
GGTGCCCCTAA

| | | | |
|---|---|---|---|
| NOV13m, | | | |
| SNP13375774 of | | | |
| CG51622-01, | SEQ ID NO: 394 | | MW at 34552.3kD |
| Protein Sequence | SNP Pos: 83 | 320 aa | SNP Change: His to Arg |

MAGPWTFTLLCGLLAATLIQATLSPTAVLILGPKVIKESLTQELKDHNATSILQQLPLLSAMREKPAG
GIPVLGSLVNTVLKRITPSRLKVITANILQLQVKPSANDQELLVKIPLDMVAGFNTPLVKTIVEFHNT
TEAQATIRMDTSASGPTRLVLSDCATSHGSLRIQLLHKLSFLVNALAKQVMNLLVPSLPNLVKNQLCP
VIEASFNGMYADLLQLVKGRCSALSPTFSFTTELASRPGKVTKWFNNSAASLTMPTLDNIPFSLIVSQ
DVVKAAVAAVLSPEEFMVLLDSVVNLSTRQRIGPPRPHHRNFLNTGCP

| | | |
|---|---|---|
| NOV13n, | | |
| SNP1 3375775 of | SEQ ID NO: 395 | 963 bp |
| CG51622-01, | ORF Start: ATG at 1 | ORF Stop: TAA at 961 |
| DNA Sequence | SNP Pos: 250 | SNP Change: A to G |

ATGGCCGGCCCGTGGACCTTCACCCTTCTCTGTGGTTTGCTGGCAGCCACCTTGATCCAAGCCACCCT
CAGTCCCACTGCAGTTCTCATCCTCGGCCCAAAAGTCATCAAAGAAAGTCTGACACAGGAGCTGAAGG
ACCACAACGCCACCAGCATCCTGCAGCAGCTGCCGCTGCTCAGTGCCATGCGGGAAAAGCCAGCCGGA
GGCATCCCTGTGCTGGGCAGCCTGGTGAACACCGTCCTGAAGCACGTCACCCCATCCAGGCTGTTGGT
CATCACAGCTAACATCCTCCAGCTGCAGGTGAAGCCCTCGGCCAATGACCAGGAGCTGCTAGTCAAGA
TCCCCCTGGACATGGTGGCTGGATTCAACACGCCCCTGGTCAAGACCATCGTGGAGTTCCACATGACG
ACTGAGGCCCAAGCCACCATCCGCATGGACACCAGTGCAAGTGGCCCCACCCGCCTGGTCCTCAGTGA
CTGTGCCACCAGCCATGGGAGCCTGCGCATCCAACTGCTGCATAAGCTCTCCTTCCTGGTGAACGCCT
TAGCTAAGCAGGTCATGAACCTCCTAGTGCCATCCCTGCCCAATCTAGTGAAAAACCAGCTGTGTCCC
GTGATCGAGGCTTCCTTCAATGGCATGTATGCAGACCTCCTGCAGCTGGTGAAGGGTAGGTGCTCTGC
TCTCTCTCCCACTTTTTCCTTTACTACGGAGCTGGCCTCCAGACCCGGAAAGGTGACCAAGTGGTTCA
ATAACTCTGCAGCTTCCCTGACAATGCCCACCCTGGACAACATCCCGTTCAGCCTCATCGTGAGTCAG
GACGTGGTGAAAGCTGCAGTGGCTGCTGTGCTCTCTCCAGAAGAATTCATGGTCCTGTTGGACTCTGT
GGTAAACCTCAGCACAAGGCAGAGAATAGGGCCGCCCAGGCCACATCATAGGAATTTCCTGAACACAG
GGTGCCCCTAA

| | | | |
|---|---|---|---|
| NOV13n, | | | |
| SNP13375775 of | | | |
| CG51622-01, | SEQ ID NO: 396 | | MW at 3451 9.2kD |
| Protein Sequence | SNP Pos: 84 | 320 aa | SNP Change: Ile to Val |

MAGPWTFTLLCGLLAATLIQATLSPTAVLILGPKVIKESLTQELKDHNATSILQQLPLLSAMREKPAG
GIPVLGSLVNTVLKHVTPSRLKVITANILQLQVKPSANDQELLVKIPLDMVAGFNTPLVKTIVEFHMT
TEAQATIPMDTSAsGPTRLVLSDCATSHGSLRIQLLHKLSFLVNALAKQVNNLLVPSLPNLVKNQLCP
VIEASFNGMYADLLQLVKGRCSALSPTFSFTTELASRPGKVTKWFNNSAASLTMPTLDNIPFSLIVSQ
DVVKAAVAAVLSPEEFMVLLDSVVNLSTRQRIGPPRPHHRNFLNTGCP

A ClustalW comparison of the above protein sequences yields the following sequence alignment shown in Table 13B.

TABLE 13B
Comparison of the NOV13 protein sequences.

| | |
|---|---|
| NOV13a | MGVEDSQRRDPGRGLASCTCCPLTPGKMAGPWTFTLLCGLLAATLIQATLSPTAVLILGP |
| NOV13b | ---------------------------MAGPWTFTPLCGLLAATLIQATLSPTAVLILGP |
| NOV13c | ---------------------------MAGPWTFTLLCGLLAATLIQATLSPTAVLILGP |
| NOV13d | ---------------------------MAGPWTFTLLCGLLAATLIQATLSPTAVLILGP |
| NOV13e | -----------------------TGSTMAGPWTFTLLCGLLAATLIQATLSPTAVLILGP |
| NOV13f | ------------------------------------------------TGSPTAVLILGP |
| NOV13g | ---------------------------MAGPWTFTLLCGLLAATLIQATLSPTAVLILGP |
| NOV13h | ---------------------------MAGPWTFTLLCGLLAATLIQATLSPTAVLILGP |
| NOV13i | ---------------------------MAGPWTFTLLCGLLAATLIQATLSPTAVLILGP |
| NOV13j | ---------------------------MAGPWTFTLLCGLLAATLIQATLSPTAVLILGP |
| NOV13k | ---------------------------------------------------PTAVLILGP |
| NOV13l | ---------------------------MAGPWTFTLLCGLLAATLIQATLSPTAVLILGP |
| NOV13a | KVIKEKLTQELKDHNATSILQQLPLLSAMREKPAGGIPVLGSLVNTVLKHII--WLKVIT |
| NOV13b | KVIKEKLTQELKDHNATSILQQLPLLSAMREKPAGGIPVLGSLVNTVLKHVI--WLKVIT |
| NOV13c | KVIKEKLTQELKDHNATSILQQLPLLSAMREKPAGGIPVLGSLVNTVLKHVI--WLKVIT |
| NOV13d | KVIKESLTQELKDHNATSILQQLPLLSAMREKPAGGIPVLGSLVNTVLKHITPSRLKVIT |
| NOV13e | KVIKEKLTQELKDHNATSILQQLPLLSAMREKPAGGIPVLGSLVNTVLKHVI--WLKVIT |
| NOV13f | KVIKEKLTQELKDHNATSILQQLPLLSAMREKPAGGIPVLGSLVNTVLKHVI--WLKVIT |
| NOV13g | KVIKEKLTQELKDHNATSILQQLPLLSAMREKPAGGIPVLGSLVNTVLKHII--WLKVIT |
| NOV13h | KVIKEKLTQELKDHNATSILQQLPLLSAMREKPAGGIPVLGSLVNTVLKHII--WLKVIT |
| NOV13i | KVIKEKLTQELKDHNATSILQQLPLLSAMREKPAGGIPVLGSLVNTVLKHVI--WLKVIT |
| NOV13j | KVIKEKLTQELKDHNATSILQQLPLLSAMREKPAGGIPVLGSLVNTVLKHII--WLKVIT |
| NOV13k | KVIKEKLTQELKDHNATSILQQLPLLSAMREKPAGGIPVLGSLVNTVLKHII--WLKVIT |
| NOV13l | KVIKEKLTQELKDHNATSILQQLPLLSAMREKPAGGIPVLGSLVNTVLKHVI--WLKVIT |
| NOV13a | ANILQLQVKPSANDQELLVKIPLDMVAGFNTPLVKTIVEFHMTTEAQATIRMDTSASGPT |
| NOV13b | ANILQLQVKPSANDQELLVKIPLDMVAGFNT----------------------------- |
| NOV13c | ANILQLQVKPSANDQELLVKIPLDMVAGFNTPLVKTIVEFHMTTEAQATIRMDTSASGPT |
| NOV13d | ANILQLQVKPSANDQELLVKIPLDMVAGFNTPLVKTIVEFHMTTEAQATIRMDTSASGPT |
| NOV13e | ANILQLQVKPSANDQELLVKIPLDMVAGFNTPLVKTIVEFHMTTEAQATIRMDTSASGPT |
| NOV13f | ANILQLQVKPSANDQELLVKIPLDMVAGFNTPLVKTIVEFHMTTEAQATIRMDTSASGPT |
| NOV13g | ANILQLQVKPSANDQELLVKIPLDMVAGFNTPLVKTIVEFHMTTEAQATIRMDTSASGPT |
| NOV13h | ANILQLQVKPSANDQELLVKIPLDMVAGFNTPLVKTIVEFHMTTEAQATIRMDTSASGPT |
| NOV13i | ANILQLQVKPSANDQELLVKIPLDMVAGFNTPLVKTIVEFHMTTEAQATIRMDTSASGPT |
| NOV13j | ANILQLQVKPSANDQELLVKIPLDMVAGFNTPLVKTIVEFHMTTEAQATIRMDTSASGPT |
| NOV13k | ANILQLQVKPSANDQELLVKIPLDMVAGFNTPLVKTIVEFHMTTEAQATIRMDTSASGPT |
| NOV13l | ANILQLQVKPSANDQELLVKIPLDMVAGFNTPLVKTIVEFHMTTEAQATIRMDTSASGPT |
| NOV13a | RLVLSDCATSHGSLRIQLLHKLSFLVNALAKQVMNLLVPSLPNLVKNQLCPVIEASFNGM |
| NOV13b | ---------------------LSFLVNALAKQVMNLLVPSLPNLVKNQLCPVIEASFNGM |
| NOV13c | RLVLSDCATSHGSLRIQLLHKLSFLVNALAKQVMNLLVPSLPNLVKN------------- |
| NOV13d | RLVLSDCATSHGSLRIQLLHKLSFLVNALAKQVMNLLVPSLPNLVKNQLCPVIEASFNGM |
| NOV13e | RLVLSDCATSHGSLRIQLLHKLSFLVNALAKQVMNLLVPSLPNLVKNQLCPVIEASFNGM |
| NOV13f | RLVLSDCATSHGSLRIQLLHKLSFLVNALAKQVMNLLVPSLPNLVKNQLCPVIEASFNGM |
| NOV13g | RLVLSDCATSHGSLRIQLLHKLSFLVNALAKQVMNLLVPSLPNLVKNQLCPVIEASFNGM |
| NOV13h | RLVLSDCATSHGSLRIQLLHKLSFLVNALAKQVMNLLVPSLPNLVKNQLCPVIEASFNGM |
| NOV13i | RLVLSDCATSHGSLRIQLLHKLSFLVNALAKQVMNLLVPSLPNLVKNQLCPVIEASFNGM |
| NOV13j | RLVLSDCATSHGSLRIQLLHKLSFLVNALAKQVMNLLVPSLPNLVKNQLCPVIEASFNGM |
| NOV13k | RLVLSDCATSHGSLRIQLLHKLSFLVNALAKQVMNLLVPSLPNLVKNQLCPVIEASFNGM |
| NOV13l | RLVLSDCATSHGSLRIQLLHKLSFLVNALAKQVMNLLVPSLPNLVKNQLCPVIEASFNGM |
| NOV13a | YADLLQLVKVPISLSIDRLEFDLLYPAIKGDTIQLYLGAKLLDSQGKVTKWFNNSAASLT |
| NOV13b | YADLLQLVKVPISLSIDRLEFDLLYPAIKGDTIQLYLGAKLLDSQGKVTKWFNNSAASLT |
| NOV13c | -----QLVKVPISLSIDRLEFDLLYPAIKGDTIQLYLGAKLLDSQGKVTKWFNNSAASLT |
| NOV13d | YADLLQLVKGRCSALSPTFSFTTELASRP------------G----KVTKWFNNSAASLT |
| NOV13e | YADLLQLVKVPISLSIDRLEFDLLYPAIKGDTIQLYLGAKLLDSQGKVTKWFNNSAASLT |
| NOV13f | YADLLQLVKVPISLSIDRLEFDLLYPAIKGDTIQLYLGAKLLDSQGKVTKWFNNSAASLT |
| NOV13g | YADLLQLVKGRCSALSPTFSFTTELASRP------------G----KVTKWFNNSAASLT |
| NOV13h | YADLLQLVKVPISLSIDRLEFDLLYPAIKGDTIQLYLGAKLLDSQGKVTKWFNNSAASLT |
| NOV13i | YADLLQLVKVPISLSIDRLEFDLLYPAIKGDTIQLYLGAKLLDSQGKVTKWFNNSAASLT |
| NOV13j | YADLLQLVKVPISLSIDRLEFDLLYPAIKGDTIQLYLGAKLLDSQGKVTKWFNNSAASLT |
| NOV13k | YADLLQLVKVPISLSIDRLEFDLLYPAIKGDTIQLYLGAKLLDSQGKVTKWFNNSAASLT |
| NOV13l | YADLLQLVKVPISLSIDRLEFDLLYPAIKGDTIQLYLGAKLLDSQGKVTKWFNNSAASLT |
| NOV13a | MPTLDNIPFSLIVS--------QDVVKAAVAAVLSPEEFMVLLDSVLPESAHRLKSSIGL |
| NOV13b | MPTLDNIPFSLIVS--------QDVVKAAVAAVLSPEEFMVLLDSVLPESAHRLKSSIGL |
| NOV13c | MPTLDNIPFSLIVS--------QDVVKAAVAAVLSPEEFMVLLDSVLPESAHRLKSSIGL |
| NOV13d | MPTLDNIPFSLIVS--------QDVVKAAVAAVLSPEEFMVLLDSVVNLSTRQRIG---- |
| NOV13e | MPTLDNIPFSLIVS--------QDVVKAAVAAVLSPEEFMVLLDSVVNLSTRQRIG---- |
| NOV13f | MPTLDNIPFSLIVS--------QDVVKAAVAAVLSPEEFMVLLDSVVNLSTRQRIG---- |
| NOV13g | MPTLDNIPFSLIVS--------QDVVKAAVAAVLSPEEFMVLLDSVVNLSTRQRIG---- |
| NOV13h | MPTLDNIPFSLIVSHPFSLIVSQDVVKAAVAAVLSPEEFMVLLDSVLPESAHRLKSSIGL |
| NOV13i | MPTLDNIPFSLIVS--------QDVVKAAVAAVLSPEEFMVLLDSVVNLSTRQRIG---- |
| NOV13j | MPTLDNIPFSLVS---------QDVVKAAVAAVLSPEEFMVLLDSVLPESAHRLKSSIGL |
| NOV13k | MPTLDNIPFSLIVS--------QDVVKAAVAAVLSPEEFMVLLDSVLPESAHRLKSSIGL |
| NOV13l | MPTLDNIPFSLIVS--------QDVVKAAVAAVLSPEEFMVLLDSVLPESAHRLKSSIGL |
| NOV13a | INEKAADKLGSTQIVKILTQDTPEFFIDQGHAKVAQLIVLEVFPSSEALRPLFTLGIEAS |
| NOV13b | INEKAADKLGSTQIVKILTQDTPEFFIDQGHAKVAQLIVLEVFPSSEALRPLFTLGIEAS |
| NOV13c | INEKAADKLGSTQIVKILTQDTPEFFIDQGHAKVAQLIVLEVFPSSEALRPLFTLGIEAS |
| NOV13d | ----------------------P--------------------P-----RP--------- |
| NOV13e | ----------------------P--------------------P-----RP--------- |
| NOV13f | ----------------------P--------------------P-----RP--------- |
| NOV13g | ----------------------P--------------------P-----RP--------- |
| NOV13h | INEK-----------------------------------------------------EAS |
| NOV13i | ----------------------P--------------------P-----RP--------- |
| NOV13j | INEKAADKLGSTQIVKILTQDTPEFFIDQGHAKVAQLIVLEVFPSSEALRPLFTLGIEAS |
| NOV13k | INEKAADKLGPTQIVKILTQDTPEFFIDQGHAKVAQLIVLEVFPSSEALRPLFTLGIEAS |
| NOV13l | INEKAADKLGSTQIVKILTQDTPEFFIDQGHAKVAQLIVLEVFPSSEALRPLFTLGIEAS |
| NOV13a | SEAQFYTKGDQLILNLNNISSDRIQLMNSGIGWFQCPFSLSQPDVLKNIITEIIHSILLP |
| NOV13b | SEAQFYTKGDQLILNLNNISSDRIQLMNSGIGWFQ-------PDVLKNIITEIIHSILLP |
| NOV13c | SEAQFYTKGDQLILNLNNISSDRIQLMNSGIGWFQ-------PDVLKNIITEIIHSILLP |
| NOV13d | HHRNFLNTGCP------------------------------------------------- |
| NOV13e | HHRNFLNTGCPLEG---------------------------------------------- |
| NOV13f | HHRNFLNTGCPLE----------------------------------------------- |
| NOV13g | HHRNFLNTGCP------------------------------------------------- |
| NOV13h | SEAQFYTKGDQLILNLNNISSDRIQLMNSGIGWFQ-------PDVLKNIITEIIHSILLP |
| NOV13i | HHRNFLNTGCP------------------------------------------------- |
| NOV13j | SEAQFYTKGDQLILNLNNISSDRIQLMNSGIGWFQ-------PDVLKNIITEIIHSILLP |
| NOV13k | SEAQFYTKGDQLILNLNNISSDRIQLMNSGIGWFQ-------PDVLKNIITEIIHSILLP |
| NOV13l | SEAQFYTKGDQLILNLNNISSDRIQLMNSGIGWFQ-------PDVLKNIITEIIHSILLP |
| NOV13a | NQNGKLRSGVPVSLVKALGFEAAESSLTKDALVLTPASLWKPSSPVSQ |
| NOV13b | NQNGKLRSGVPVSLVKALGFEAAESSLSKDALVLTPASLWKPTSPVSQ |
| NOV13c | NQNGKLRSGVPVSLVKALGFEAAESSLSKDALVLTPASLWKPTSPVSQ |
| NOV13d | ------------------------------------------------ |
| NOV13e | ------------------------------------------------ |
| NOV13f | ------------------------------------------------ |
| NOV13g | ------------------------------------------------ |
| NOV13h | NQNGKLRSGVPVSLVKALGFEAAESSLTKDALVLTPASLWKPSSPVSQ |
| NOV13i | ------------------------------------------------ |
| NOV13j | NQNGKLRSGVPVSLVKALGFEAAESSLTKDALVLTPASLWKPSSPVSQ |
| NOV13k | NQNGKLRSGVPVSLVKALGFEAAESSLTKDALVLTPASLWKPSSPVSQ |
| NOV13l | NQNGKLRSGVPVSLVKALGFEAAESSLSKDALVLTPASLWKPTSPVSQ |
| NOV13a | (SEQ ID NO: 370) |
| NOV13b | (SEQ ID NO: 372) |
| NOV13c | (SEQ ID NO: 374) |
| NOV13d | (SEQ ID NO: 376) |
| NOV13e | (SEQ ID NO: 378) |
| NOV13f | (SEQ ID NO: 380) |
| NOV13g | (SEQ ID NO: 382) |
| NOV13h | (SEQ ID NO: 384) |
| NOV13i | (SEQ ID NO: 386) |
| NOV13j | (SEQ ID NO: 388) |
| NOV13k | (SEQ ID NO: 390) |
| NOV13l | (SEQ ID NO: 392) |

Further analysis of the NOV13a protein yielded the following properties shown in Table 13C.

TABLE 13C
Protein Sequence Properties NOV13a

| | |
|---|---|
| SignalP analysis: | Cleavage site between residues 52 and 53 |
| PSORT II analysis: | |

| PSG: | a new signal peptide prediction method | |
|---|---|---|
| | N-region: | length 10; pos. chg 2; neg. chg 3 |
| | H-region: | length 2; peak value 0.00 |
| | PSG score: | −4.40 |

| | |
|---|---|
| GvH: | von Heijne's method for signal seq. recognition |
| | GvH score (threshold: −2.1): 1.17 |
| | possible cleavage site: between 48 and 49 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 2 | | |
| | Number of TMS(s) for threshold 0.5: 1 | | |
| | INTEGRAL | Likelihood = | Transmembrane 34-50 |
| | | −2.71 | |

| | |
|---|---|
| PERIPHERAL | Likelihood = 1.16 (at 200) |

| | |
|---|---|
| | ALOM score: −2.71 (number of TMSs: 1) |
| MTOP: | Prediction of membrane topology (Hartmann et al.) |
| | Center position for calculation: 41 |
| | Charge difference: −0.5 C(1.5)-N(2.0) |
| | N >= C: N-terminal side will be inside |

>>> membrane topology: type 2 (cytoplasmic tail 1 to 34)

| MITDISC: | discrimination of mitochondrial targeting seq | | | |
|---|---|---|---|---|
| | R content: | 0 | Hyd Moment(75): | 8.50 |
| | Hyd Moment(95): | 5.94 | G content: | 1 |
| | D/E content: | 2 | S/T content: | 0 |

| | |
|---|---|
| | Score: −7.21 |
| Gavel: | prediction of cleavage sites for mitochondrial preseq |
| | cleavage site motif not found |

NUCDISC: discrimination of nuclear localization signals

| | |
|---|---|
| | pat4: none |
| | pat7: none |
| | bipartite: none |
| | content of basic residues: 8.3% |
| | NLS Score: −0.47 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: MGVEDSQ
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 76.7 |
| COIL: | Lupas's algorithm to detect coiled-coil regions |
| | total: 0 residues |

Final Results (k = 9/23):

```
30.4%: mitochondrial
26.1%: cytoplasmic
17.4%: Golgi
  8.7%: endoplasmic reticulum
  4.3%: vacuolar
  4.3%: extracellular, including cell wall
  4.3%: nuclear
  4.3%: vesicles of secretory system
```

>> prediction for CG51622-04 is mit (k = 23)

A search of the NOV13a protein against the Geneseq database, a proprietary database that contains sequences published in patents and patent publication, yielded several homologous proteins shown in Table 13D.

TABLE 13D
Geneseq Results for NOV13a

| | | NOV13a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAY77126 | Human neurotransmission-associated | 28 . . . 518 | 484/491 (98%) | 0.0 |
| | protein (NTAP) 2799056 - Homo | 1 . . . 484 | 484/491 (98%) | |
| | sapiens, 484 aa. [WO200001821-A2, | | | |
| | 13-JAN-2000] | | | |
| AAG63976 | Amino acid sequence of a human | 28 . . . 518 | 483/491 (98%) | 0.0 |
| | Lng103 polypeptide - Homo sapiens, | 1 . . . 484 | 484/491 (98%) | |
| | 484 aa. [WO200161055-A2, 23-AUG- | | | |
| | 2001] | | | |
| ABU58539 | Human PRO polypeptide #140 - Homo | 28 . . . 518 | 483/491 (98%) | 0.0 |
| | sapiens, 484 aa. [US2003027272-A1, | 1 . . . 484 | 484/491 (98%) | |
| | 06-FEB-2003] | | | |
| ABU57070 | Human PRO polypeptide #140 - Homo | 28 . . . 518 | 483/491 (98%) | 0.0 |
| | sapiens, 484 aa. [US2003027280-A1, | 1 . . . 484 | 484/491 (98%) | |
| | 06-FEB-2003] | | | |
| ABU56075 | Human secreted/transmembrane | 28 . . . 518 | 483/491 (98%) | 0.0 |
| | protein, PRO1357 - Homo sapiens, 484 | 1 . . . 484 | 484/491 (98%) | |
| | aa. [US2003022298-A1, 30-JAN-2003] | | | |

In a BLAST search of public sequence databases, the NOV13a protein was found to have homology to the proteins shown in the BLASTP data in Table 13E.

TABLE 13E
Public BLASTP Results for NOV13a

| | | NOV13a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8TDL5 | Von Ebner minor protein - Homo | 28 . . . 518 | 484/491 (98%) | 0.0 |
| | sapiens (Human), 484 aa. | 1 . . . 484 | 484/491 (98%) | |
| Q96HK6 | Similar to DNA segment, Chr 2, | 28 . . . 518 | 481/491 (97%) | 0.0 |
| | Massachusetts Institute of Technology | 1 . . . 484 | 484/491 (97%) | |
| | 19 - Homo sapiens (Human), 484 aa. | | | |
| Q9BWZ6 | DJ1187J4.1.1 (Novel protein similar to | 227 . . . 518 | 285/292 (97%) | e−154 |
| | mouse von Ebner salivary gland | 1 . . . 285 | 285/292 (97%) | |
| | protein, isoform 1.) - Homo sapiens | | | |
| | (Human), 285 aa (fragment). | | | |
| CAD13090 | Sequence 15 from Patent WO0174851 - | 28 . . . 337 | 276/310 (89%) | e−146 |
| | Homo sapiens (Human), 318 aa. | 1 . . . 294 | 278/310 (89%) | |
| Q8SPF8 | Von Ebner minor salivary gland protein- | 28 . . . 508 | 269/481 (55%) | e−144 |
| | like protein - Bos taurus (Bovine), 473 aa. | 1 . . . 472 | 353/481 (72%) | |

PFam analysis predicts that the NOV13a protein contains the domains shown in the Table 13F.

TABLE 13F
Domain Analysis of NOV13a

| | | | |
|---|---|---|---|
| Pfam | NOV13a | Identities/ | Expect |
| Domain | Match Region | Similarities | Value |
| | | for the | |
| | | Matched Region | |

### Example 14.

The NOV14 clone was analyzed, and the nucleotide and encoded polypeptide sequences are shown in Table 14A.

TABLE 14A
NOV14 Sequence Analysis

| | | |
|---|---|---|
| NOV14a, CG51821-01 | SEQ ID NO: 397 | 5103 bp |
| DNA Sequence | ORF Start: ATG at 1 | ORF Stop: TGA at 5101 |

ATGGGCTTCTTGCCCAAGCTTCTCCTCCTGGCCTCAGCCGTTCTTCCCCCAGGCCAGGCCTCATGGGG
CGTCTCCAGTCCCCAGGACGTGCAGGGTGTGAAGGGGTCTTGCCTGCTTATCCCCTGCATCTTCAGCT
TCCCTGCCGACGTGGAGGTGCCCGACGGCATCACGGCCATCTGGTACTACGACTACTCGGGCCAGCGG
CAGGTGGTGAGCCACTCGGCGGACCCCAAGCTGGTGGAGGCCCGCTTCCGCGGCCGCACCGAGTTCAT
ACAACTTCCGCTTCGAGATCAGTGAGGTCAACCGCTGGTCAGATGTGAAAGGCACCTTGGTCACAGTA
ACAGGTGATCCCAGGGTGCCCACCATTGCCTCCCCGGTGGAGCTTCTCGAGGGCACAGAGGTGGACTT
CAACTGCTCCACTCCCTACGTATGCCTGCAGGAGCAGGTCAGACTGCAGTGGCAAGGCCAGGACCCTG
CTCGCTCTGTCACCTTCAACAGCCAGAAGTTTGAGCCCACCGGCGTCGGCCACCTGGAGACCCTCCAC
ATGGCCATGTCCTGGCAGGACCACGGCCGGATCCTGCGCTGCCAGCTCTCCGTGGCCAATCACAGGGC
TCAGAACGAGATTCACCTCCAAGTGAAGTGTGCCCCCAAGGGTGTGAAGATCCTCCTCAGCCCCTCGG
GGAGGAACATCCTTCCAGGTGAGCTGGTCACACTCACCTGCCAGGTGAACAGCAGCTACCCTGCAGTC
AGTTCCATTAAGTGGCTCAAGGATGGGGTACGCCTCCAAACCAAGACTGGTGTGCTGCACCTGCCCCA
GGCAGCCTGGAGCGATGCTGGCGTCTACACCTGCCAAGCTGAGAACGGCGTGGGCTCTTTGGTCTCAC
CCCCCATCAGCCTCCACATCTTCGTGGCTGAGGTCCAGGTGAGCCCAGCAGGTCCCATCCTGGAGAAC
CAGACAGTGACACTAGTCTGCAACACACCCAATGAGGCACCCAGTGATCTCCGCTACAGCTGGTACAA
GAACCATGTCCTGCTGGAGGATGCCCACTCCCATACCCTCCGGCTGCACTTGGCCACTAGGGCTGATA
CTGGCTTCTACTTCTGTGAGGTGCAGAACGTCCATGGCAGCGAGCGCTCGGGCCCTGTCAGCGTGGTA
GTCACAGACCCGCCTCTCACTCCAGTCCTGACAGCCTTCCTGGAGACCCAGGCGGGACTTGTGGGCAT
CCTTCACTGCTCTGTGGTCAGTGAGCCCCTGGCCACACTGGTGCTGTCACATGGGGGTCATATCCTGG
CCTCCACCTCCGGGGACAGTGATCACAGCCCACGCTTCAGTGGTACCTCTGGTCCCAACTCCCTGCGC
CTGGAGATCCGAGACCTGGAGGAAACTGACAGTGGGGAGTACAAGTGCTCAGCCACCAACTCCCTTGG
AAATGCAACCTCCACCCTGGACTTCCATGCCAATGTCGCCCGTCTCCTCATCAGCCCGGCAGCCGAGG
TGGTGGAAGGACAGGCAGTGACACTGAGCTGCAGAAGCGGCCTAAGCCCCACACCTGATGCCCGCTTC
TCCTGGTACCTGAATGGAGCCCTGCTTCACGAGGGTCCCGGCAGCAGCCTCCTGCTCCCCGCGGCCTC
CAGCACTGACGCCGGCTCATACCACTGCCGGGCCCGGGACGGCCACAGTGCCAGTGGCCCCTCTTCGC
CAGCTGTTCTCACTGTGCTCTGTGAGCAGCCACCACGACAACCAACATTCACCACCAGGCTGGACCTT
GATGCCGCTGGGGCCGGGGCTGGACGGCGAGGCCTCCTTTTGTGCCGTGTGGACAGCGACCCCCCCGC
CAGGCTGCAGCTGCTCCACAAGGACCGTGTTGTGGCCACTTCCCTGCCATCAGGGGGTGGCTGCAGCA
CCTGTGGGGGCTGTTCCCCACGCATGAAGGTCACCAAAGCCCCCAACTTGCTGCGTGTGGAGATTCAC
AACCCTTTGCTGGAAGAGGAGGGCTTGTACCTCTGTGAGGCCAGCAATGCCCTGGGCAACGCCTCCAC
CTCAGCCACCTTCAATGGCCAGGCCACTGTCCTGGCCATTGCACCATCACACACACTTCAGGAGGGCA
AGAAGCACAACTTGACTTGCAACGTGAGCCGGGAAGCTGCTGGCAGCCCTGCTAACTTCTCCTGGTTC
CGAAATGGGGTGCTGTGGGCCCAGGGTCCCCTGGAGACCGTGACACTGCTGCCCGTGGCCAGAACTGA
TGCTGCCCTTTACGCCTGCCGCATCCTGACTGAGGCTGGTGCCCAGCTCTCCACTCCCGTGCTCCTGA
GTGTACTCTATCCCCCGGACCGTCCAAAGCTGTCAGCCCTCCTAGACATGGGCCAGGGCCACATGGCT
CTGTTCATCTGCACTGTGGACAGCCGCCCCCTGGCCTTGCTGGCCTTGTTCCATGGGGAGCACCTCCT
GGCCACCAGCCTGGGTCCCCAGGTCCCATCCCATGGTCGGTTCCAGGCTAAAGCTGAGGCCAACTCCC
TGAAGTTAGAGGTCCGAGAACTGGGCCTTGGGGACTCTGGCAGCTACCGCTGTGAGGCCACAAATGTT
TGAGCTCCAAGAGGGCCAGGCTGTGGTCCTGAGCTGCCAGGTACACACAGGAGTCCCAGAGGGGACCT
CATATCGTTGGTATCGGGATGGCCAGCCCCTCCAGGAGTCGACCTCGGCCACGCTCCGCTTTGCAGCC
AGCTGCACCCATCAGCCTCCACGTGTCCTGTAAGGATGCCCCACGCCACGTCACACTCACTACCCTGA
TGGACACAGGCCCTGGACGACTGGGCCTCCTCCTGTGCCGTGTGGACAGTGACCCTCCGGCCCAGCTG
TCCCAGGCTGCATGTGGCTGTGGCCCCCAACACACTGCGTCTGGAGATCCACGGGGCTATGCTGGAGG
ATGAGGGTGTCTATATCTGTGAGGCCTCCAACACCCTGGGCCAGGCCTCGGCCTCAGCTGACTTCGAC
GCTCAAAGCTGTGAATGTGCAGGTGTGGCCCGGGGCTACCGTGCGGGAGGGGCAGCTGGTGAACCTGA
CCTGCGCCTGACCTACCTCCTGGAGAGCCATGGCGGGCAGCTGGCCCTGGTACTGTGCACTGTGGACA
GCCGCCCGCCCGCCCAGCTGGCCCTCAGCCACGCCGGTCGCCTCTTGGCCTCCTCGACAGCAGCCTCT
GTCCCCAACACCCTGCGCCTGGAGCTGCGAGGGCCACAGCCCAGGGATGAGGGTTTCTACAGCTGCTC
TGCCCGCAGCCCTCTGGGCCAGGCCAACACGTCCCTGGAGCTGCGGCTGGAGGTGCGGGTGATCCTGG
CTCCGGAGGCTGCCGTGCCTGAAGGTGCCCCCATCACAGTGACCTGTGCGGACCCTGCTGCCCACGCA
CCCACACTCTATACTTGGTACCACAACGGTCGTTGGCTGCAGGAGGGTCCAGCTGCCTCACTCTCATT
CCTGGTGGCCACGCGGGCTCATGCAGGCGCCTACTCTTGCCAGGCCCAGGATGCCCAGGGCACCCGCA
GCTCCCGTCCTGCTGCCCTGCAAGTCCTCTGTGCCCCTCAGGACGCTGTCCTGTCCTCCTTCCGGGAC
TCCAGGGCCAGATCCATGGCTGTGATACAGTGCACTGTGGACAGTGAGCCACCTGCTGAGCTGGCCCT
ATCTCATGATGGCAAGGTGCTGGCCACGAGCAGCGGGGTCCACAGCTTGGCATCAGGGACAGGCCATG
TCCAGGTGGCCCGAAACGCCCTACGGCTGCAGGTGCAAGATGTGCCTGCAGGTGATGACACCTATGTT
TGCACAGCCCAAAACTTGCTGGGCTCAATCAGCACCATCGGGCGGTTGCAGGTAGAAGGTGAGTGGCG
CGTGGTGGCAGAGCCTGGCCTGGACGTGCCTGAGGGCGCTGCCCTGAACCTCAGCTGCCGCCTCCTGG
GTGGCCCTGGGCCTGTGGGCAACTCCACCTTTGCATGGTTCTGGAATGACCGGCGGCTGCACGCGGAG
CCTGTGCCCACTCTCGCCTTCACCCACGTGGCTCGTGCTCAAGCTGGGATGTACCACTGCCTGGCTGA
GCTCCCCACTGGGGCTGCTGCCTCTGCTCCAGTCATGCTCCGTGTGCTCTACCCTCCCAAGACGCCCA
CCATGATGGTCTTCGTGGAGCCTGAGGGTGGCCTCCGGGGCATCCTGGATTGCCGAGTGGACAGCGAG
CCGCTCGCCAGCCTGACTCTCCACCTTGGCAGTCGACTGGTGGCCTCCAGTCAGCCCCAGGGTGCTCC
TGCAGAGCCACACATCCATGTCCTGGCTTCCCCCAATGCCCTGAGGGTGGACATCGAGGCGCTGAGGC
CCAGCGACCAAGGGGAATACATCTGTTCTGCCTCAAATGTCCTGGGCCCTGCCTCTACCTCCACCTAC
TTTGGGGTCAGAGCCCTGCACCGCCTGCATCAGTTCCAGCAGCTGCTCTGGGTCCTGGGACTGCTGGT
GGGCCTCCTGCTCCTGCTGTTGGGCCTGGGGGCCTGCTACACCTGGAGAAGGAGGCGTGTTTGTAAGC
AGAGCATGGGCGAGAATCCAGGGCGGGCATCGTCTTTCCATTTACTGCCTCTAGCTGGGTCTTCAAGG
TGA

| | | | |
|---|---|---|---|
| NOV14a, CG51821-01 | | | |
| Protein Sequence | SEQ ID NO: 398 | 1700 aa | MW at 180833.0kD |

MGFLPKLLLLASAVLPPGQASWGVSSPQDVQGVKGSCLLIPCIFSFPADVEVPDGITAIWYYDYSGQR
QVVSHSADPKLVEARFRGRTEFMGNPEHRVCNLLLKDLQPEDSGSYNFRFEISEVNRWSDVKGTLVTV
TGDPRVPTIASPVELLEGTEVDFNCSTPYVCLQEQVRLQWQGQDPARSVTFNSQKFEPTGVGHLETLH
MAMSWQDHGRILRCQLSVANHRAQSEIHLQVKCAPKGVKILLSPSGRNILPGELVTLTCQVNSSYPAV
SSIKWLKDGVRLQTKTGVLHLPQAAWSDAGVYTCQAENGVGSLVSPPISLHIFVAEVQVSPAGPILEN
QTVTLVCNTPNEAPSDLRYSWYKNHVLLEDAHSHTLRLHLATRADTGFYFCEVQNVHGSERSGPVSVV
VTDPPLTPVLTAFLETQAGLVGILHCSVVSEPLATLVLSHGGHILASTSGDSDHSPRFSGTSGPNSLR
LEIRDLEETDSGEYKCSATNSLGNATSTLDFHAIVARLLISPAAEVVEGQAVTLSCRSGLSPTPDARF
SWYLNGALLHEGPGSSLLLPAASSTDAGSYHCRARDGHSASGPSSPAVLTVLCEQPPRQPTFTTRLDL
DAAGAGAGRRGLLLCRVDSDPPARLQLLHKDRVVATSLPSGGGCSTCGGCSPRMKVTKAPNLLRVEIH
NPLLEEEGLYLCEASNALGNASTSATFNGQATVLAIAPSHTLQEGTEANLTCNVSREAAGSPANFSWF
RNGVLWAQGPLETVTLLPVARTDAALYACRILTEAGAQLSTPVLLSVLYPPDRPKLSALLDMGQGHMA
LFICTVDSRPLALLALFHGEHLLATSLGPQVPSHGRFQAKAEANSLKLEVRELGLGDSGSYRCEATNV
LGSSNTSLFFQVRGAWVQVSPSPELQEGQAVVLSCQVHTGVPEGTSYRWYRDGQPLQESTSATLRFAA
ITLTQAGAYHCQAQAPGSATTSLAAPISLHVSCKDAPRHVTLTTLMDTGPGRLGLLLCRVDSDPPAQL
RLLHGDRLVASTLQGVGGPEGSSPRLHVAVAPNTLRLEIHGAMLEDEGVYICEASNTLGQASASADFD
AQSCECAGVARGYRAGGAAGEPDLPCVDHSPGPAHLHMVPGWAAAPGCPLHPPAQRHSQGCHLLPLRC
GPPWSGTPPLQTYHLGRPHAPRNLRLTYLLESHGGQLALvLCTVDSRPPAQLALSHAGRLLASSTAAS
VPNTLRLELRGPQPRDEGFYSCSARSPLGQANTSLELRLEVRVILAPEAAVPEGAPITVTCADPAAHA
PTLYTWYHNGRWLQEGPAASLSFLVATRAHAGAYSCQAQDAQGTRSSRPAALQVLCAPQDAVLSSFRD
SRARSMAVIQCTVDSEPPAELALSHDGKVLATSSGVHSLASGTGHVQVARNALRLQVQDVPAGDDTYV
CTAQNLLGSISTIAGRLQVEGEWRWAEPGLDVPEGAALNLSCRLLGGPGPVGNSTFAWFWNDRRLHAE
PVPTLAFTHVARAQAGMYHCLAELPTGAAASAPVMLRVLYPPKTPTMMVFVEPEGGLRGILDCRVDSE
PLASLTLHLGSRLVASSQPQGAPAEPHIHVLASPNALRVDIEALRPSDQGEYICSASNVLGPASTSTY
FGVRALHRLHQFQQLLWVLGLLVGLLLLLLGLGACYTWRRRRVCKQSMGENPGRASSFHLLPLAGSSR

| | | |
|---|---|---|
| NOV14b, 229823091 | SEQ ID NO: 399 | 717 bp |
| DNA Sequence | ORF Start: at 1 | ORF Stop: end of sequence |

AGATCTTCATGGGGCGTCTCCAGTCCCCAGGACGTGCAGGGTGTGAAGGGGTCTTGCCTGCTTATCCC
CTGCATCTTCAGCTTCCCTGCCGACGTGGAGGTGCCCGACGGCATCACGGCCATCTGGTACTACGACT
ACTCGGGCCAGCGGCAGGTGGTGAGCCACTCGGCGGACCCCAAGCTGGTGGAGGCCCGCTTCCGCGGC
CGCACCGAGTTCATGGGGAACCCCGAGCACAGGGTGTGCAACCTGCTGCTGAAGGACCTGCAGCCCGA
GGACTCTGGTTCCTACAACTTCCGCTTCGAGATCAGTGAGGTCAACCGCTGGTCAGATGTGAAAGGCA
CCTTGGTCACAGTAACAGAGGAGCCCAGGGTGCCCACCATTGCCTCCCCGGTGGAGCTTCTCGAGGGC
ACAGAGGTGGACTTCAACTGCTCCACTCCCTACGTATGCCTGCAGGAGCAGGTCAGACTGCAGTGGCA
AGGCCAGGACCCTGCTCGCTCTGTCACCTTCAACAGCCAGAAGTTTGAGCCCACCGGCGTCGGCCACC
TGGAGACCCTCCACATGGCCATGTCCTGGCAGGACCACGGCCGGATCCTGCGCTGCCAGCTCTCCGTG
GCCAATCACAGGGCTCAGAGCGAGATTCACCTCCAAGTGAAGTATGCCCCCAGGGGTGTGAAGATCCT
CCTCAGCCCCTCGGGGAGGAACATCCTTCCAGTCGAC

| | | | |
|---|---|---|---|
| NOV14b, 229823091 | | | |
| Protein Sequence | SEQ ID NO: 400 | 239 aa | Mw at 26882.1kD |

RSSWGVSSPQDVQGVKGSCLLIPCIFSFPADVEVPDGITAIWYYDYSGQRQVVSHSADPKLVEARFRG
RTEFMGNPEHRVCNLLLKDLQPEDSGSYNFRFEISEVNRWSDVKGTLVTVTEEPRVPTIASPVELLEG
TEVDFNCSTPYVCLQEQVRLQWQGQDPARSVTFNSQKFEPTGVGHLETLHMAMSWQDHGRILRCQLSV
ANHRAQSEIHLQVKYAPRGVKILLSPSGRNILPVD

| | | |
|---|---|---|
| NOV14c, 229823107 | SEQ ID NO: 401 | 717 bp |
| DNA Sequence | ORF Start: at 1 | ORF Stop: end of sequence |

AGATCTTCATGGGGCGTCTCCAGTCCCCAGGACGTGCAGGGTGTGAAGGGGTCTTGCCTGCTTATCCC
CTGCATCTTCAGCTTCCCTGCCGACGTGGAGGTGCCCGACGGCATCACGGCCATCTGGTACTACGACT
ACTCGGGCCAGCGGCAGGTGGTGAGCCACTCGGCGGACCCCAAGCTGGTGGAGGCCCGCTTCCGCGGC
CGCACCGAGTTCATGGGGAACCCCGAGCACAGGGTGTGCAACCTGCTGCTGAAGGACCTGCAGCCCGA
GGACTCTGGTTCCTACAACTTCCGCTTCGAGATCAGTGAGGTCAACCGCTGGTCAGATGTGAAAGGCA
CCTTGGTCACAGTAACAGAGGAGCCCAGGGTGCCCACCATTGCCTCCCCGGTGGAGCTTCTCGAGGGC
ACAGAGGTGGACTTCAACTGCTCCACTCCCTACGTATGCCTGCAGGAGCAGGTCAGACTGCAGTGGCA
AGGCCAGGACCCTGCTCGCTCTGTCACCTTCAACAGCCAGAAGTTTGAGCCCACCGGCGTCGGCCACC
TGGAGACCCTCCACATGGCCATGTCCTGGCAGGACCACGGCCGGATCCTGCGCTGCCAGCTCTCCATG
GCCAATCACAGGGCTCAGAGCGAGATTCACCTCCAAGTGAAGTATGCCCCCAGGGGTGTGAAGATCCT
CCTCAGCCCCTCGGGGAGGAACATCCTTCCAGTCGAC

| | | | |
|---|---|---|---|
| NOV14c, 229823107 | | | |
| Protein Sequence | SEQ ID NO: 402 | 239 aa | MW at 26914.2kD |

RSSWGVSSPQDVQGVKGSCLLIPCIFSFPADVEVPDGITAIWYYDYSGQRQVVSHSADPKLVEARFRG
RTEFMGNPEHRVCNLLLKDLQPEDSGSYNFRFETSEVNRWSDVKGTLVTVTEEPRVPTIASPVELLEG
TEVDFNCSTPYVCLQEQVRLQWQGQDPARSVTFNSQKFEPTGVGHLETLHMAMSWQDHGRILRCQLSM
ANHRAQSEIHLQVKYAPRGVKILLSPSGRNILPVD

| | | |
|---|---|---|
| NOV14d, 229823095 | SEQ ID NO: 403 | 717 bp |
| DNA Sequence | ORF Start: at 1 | ORF Stop: end of sequence |

AGATCTTCATGGGGCGTCTCCAGTCCCCAGGACGTGCAGGGTGTGAAGGGGTCTTGCCTGCTTATCCC
CTGCATCTTCAGCTTCCCTGCCGACGTGGAGGTGCCCGACGGCATCACGGCCATCTGGTACTACGACT
ACTCGGGCCAGCGGCAGGTGGTGAGCCACTCGGTGGACCCCAAGCTGGTGGAGGCCCGCTTCCGCGGC
CGCACCGAGTTCATGGGGAACCCCGAGCACAGGGTGTGCAACCTGCTGCTGAAGGACCTGCAGCCCGA
GGACTCTGGTTCCTACAACTTCCGCTTCGAGATCAGTGAGGTCAACCGCTGGTCAGATGTGAAAGGCA
CCTTGGTCACAGTAACAGAGGAGCCCAGGGTGCCCACCATTGCCTCCCCGGTGGAGCTTCTCGAGGGC
ACAGAGGTGGACTTCAACTGCTCCACTCCCTACGTATGCCTGCAGGAGCAGGTCAGACTGCAGTGGCA
AGGCCAGGACCCTGCTCGCTCTGTCACCTTCAACAGCCAGAAGTTTGAGCCCACCGGCGTCGGCCACC
TGGAGACCCTCCACATGGCCATGTCCTGGCAGGACCACGGCCGGATCCTGCGCTGCCAGCTCTCCATG
GCCAATCACAGGGCTCAGAGCGAGATTCACCTCCAAGTGAAGTATGCCCCCAAGGGTGTGAAGATCCT
CCTCAGCCCCTCGGGGAGGAACATCCTTCCAGTCGAC

| | | | |
|---|---|---|---|
| NOV14d, 229823095 | | | |
| Protein Sequence | SEQ ID NO: 404 | 239 aa | Mw at 26914.2kD |

RSSWGVSSPQDVQGVKGSCLLIPCIFSFPADVEVPDGITAIWYYDYSGQRQVVSHSVDPKLVEARFRG
RTEFMGNPEHRVCNLLLKDLQPEDSGSYNFRFEISEVNRWSDVKGTLVTVTEEPRVPTIASPVELLEG
TEVDFNCSTPYVCLQEQVRLQWQGQDPARSVTFNSQKFEPTGVGHLETLHMAMSWQDHGRILRCQLSM
ANHRAQSEIHLQVKYAPKGVKILLSPSGRNILPVD

| | | |
|---|---|---|
| NOV14e, CG51821-02 | SEQ ID NO: 405 | 717 bp |
| DNA Sequence | ORF Start: at 7 | ORF Stop: at 712 |

AGATCTTCATGGGGCGTCTCCAGTCCCCAGGACGTGCAGGGTGTGAAGGGGTCTTGCCTGCTTATCCC
CTGCATCTTCAGCTTCCCTGCCGACGTGGAGGTGCCCGACGGCATCACGGCCATCTGGTACTACGACT
ACTCGGGCCAGCGGCAGGTGGTGAGCCACTCGGCGGACCCCAAGCTGGTGGAGGCCCGCTTCCGCGGC
CGCACCGAGTTCATGGGGAACCCCGAGCACAGGGTGTGCAACCTGCTGCTGAAGGACCTGCAGCCCGA
GGACTCTGGTTCCTACAACTTCCGCTTCGAGATCAGTGAGGTCAACCGCTGGTCAGATGTGAAAGGCA
CCTTGGTCACAGTAACAGAGGAGCCCAGGGTGCCCACCATTGCCTCCCCGGTGGAGCTTCTCGAGGGC
ACAGAGGTGGACTTCAACTGCTCCACTCCCTACGTATGCCTGCAGGAGCAGGTCAGACTGCAGTGGCA
AGGCCAGGACCCTGCTCGCTCTGTCACCTTCAACAGCCAGAAGTTTGAGCCCACCGGCGTCGGCCACC
TGGAGACCCTCCACATGGCCATGTCCTGGCAGGACCACGGCCGGATCCTGCGCTGCCAGCTCTCCGTG
GCCAATCACAGGGCTCAGAGCGAGATTCACCTCCAAGTGAAGTATGCCCCCAGGGGTGTGAAGATCCT
CCTCAGCCCCTCGGGGAGGAACATCCTTCCAGTCGAC

| | | | |
|---|---|---|---|
| NOV14e, CG51821 -02 | | | |
| Protein Sequence | SEQ ID NO: 406 | 235 aa | MW at 26424.7kD |

SWGVSSPQDVQGVKGSCLLIPCIFSFPADVEVPDGITAIWYYDYSGQRQVVSHSADPKLVEARFRGRT
EFMGNPEHRVCNLLLKDLQPEDSGSYNFRFEISEVNRWSDVKGTLVTVTEEPRVPTIASPVELLEGTE
VDFNCSTPYVCLQEQVRLQWQGQDPARSVTFNSQKFEPTGVGHLETLHMAMSWQDHGRILRCQLSVAN
HRAQSEIHLQVKYAPRGVKILLSPSGRNILP

| | | |
|---|---|---|
| NOV14f, CG51821-03 | SEQ ID NO: 407 | 717 bp |
| DNA Sequence | ORF Start: at 7 | ORF Stop: at 712 |

AGATCTTCATGGGGCGTCTCCAGTCCCCAGGACGTGCAGGGTGTGAAGGGGTCTTGCCTGCTTATCCC
CTGCATCTTCAGCTTCCCTGCCGACGTGGAGGTGCCCGACGGCATCACGGCCATCTGGTACTACGACT
CGCACCGAGTTCATGGGGAACCCCGAGCACAGGGTGTGCAACCTGCTGCTGAAGGACCTGCAGCCCGA
CCTTGGTCACAGTAACAGAGGAGCCCAGGGTGCCCACCATTGCCTCCCCGGTGGAGCTTCTCGAGGGC
ACAGAGGTGGACTTCAACTGCTCCACTCCCTACGTATGCCTGCAGGAGCAGGTCAGACTGCAGTGGCA
AGGCCAGGACCCTGCTCGCTCTGTCACCTTCAACAGCCAGAAGTTTGAGCCCACCGGCGTCGGCCACC
TGGAGACCCTCCACATGGCCATGTCCTGGCAGGACCACGGCCGGATCCTGCGCTGCCAGCTCTCCATG
CCTCAGCCCCTCGGGGAGGAACATCCTTCCAGTCGAC

| | | | |
|---|---|---|---|
| NOV14f, CG51821-03 | | | |
| Protein Sequence | SEQ ID NO: 408 | 235 aa | MW at 26456.8kD |

SWGVSSPQDVQGVKGSCLLIPCIFSFPADVEVPDGITAIWYYDYSGQRQVVSHSVDPKLVEARFRGRT
EFMGNPEHRVCNLLLKDLQPEDSGSYNFRFEISEVNRWSDVKGTLVTVTEEPRVPTIASPVELLEGTE
VDFNCSTPYVCLQEQVRLQWQGQDPARSVTFNSQKFEPTGVGHLETLHMAMSWQDHGRILRCQLSMAN
HRAQSEIHLQVKYAPKGVKILLSPSGRNILP

| | | |
|---|---|---|
| NOV14g, CG51821-04 | SEQ ID NO: 409 | 5130 bp |
| DNA Sequence | ORF Start: ATG at 1 | ORF Stop: TGA at 5128 |

ATGGGCTTCTTGCCCAAGCTTCTCCTCCTGGCCTCATTCTTCCCAGCAGGCCAGGCCTCATGGGGCGT
CTCCAGTCCCCAGGACGTGCAGGGTGTGAAGGGGTCTTGCCTGCTTATCCCCTGCATCTTCAGCTTCC
CTGCCGACGTGGAGGTGCCCGACGGCATCACGGCCATCTGGTACTACGACTACTCGGGCCAGCGGCAG
GTGGTGAGCCACTCGGCGGACCCCAAGCTGGTGGAGGCCCGCTTCCGCGGCCGCACCGAGTTCATGGG
GAACCCCGAGCACAGGGTGTGCAACCTGCTGCTGAAGGACCTGCAGCCCGAGGACTCTGGTTCCTACA
ACTTCCGCTTCGAAATCAGTGAGGTCAACCGCTGGTCAGATGTGAAAGGCACCTTGGTCACAGTAACA
GAGGAGCCCAGGGTGCCCACCATTGCCTCCCCGGTGGAGCTTCTCGAGGGCACAGAGGTGGACTTCAA
CTGCTCCACTCCCTACGTATGCCTGCAGGAGCAGGTCAGACTGCAGTGGCAAGGCCAGGACCCTGCTC
GCTCTGTCACCTTCAACAGCCAGAAGTTTGAGCCCACCGGCGTCGGCCACCTGGAGACCCTCCACATG
GCCATGTCCTGGCAGGACCACGGCCGGATCCTGCGCTGCCAGCTCTCCGTGGCCAATCACAGGGCTCA
GAGCGAGATTCACCTCCAAGTGAAGTATGCCCCCAAGGGTGTGAAGATCCTCCTCAGCCCCTCGGGGA
GGAACATCCTTCCAGGTGAGCTGGTCACACTCACCTGCCAGGTGAACAGCAGCTACCCTGCAGTCACT
TCCATTAAGTGGCTCAAGGATGGGGTACGCCTCCAAACCAAGACTGGTGTGCTGCACCTGCCCCAGGC
AGCCTGGAGCGATGCTGGCGTCTACACCTGCCAAGCTGAGAACGGCGTGGGCTCTTTGGTCTCACCCC
CCATCAGCCTCCACATCTTCATGGCTGAGGTCCAGGTGAGCCCAGCAGGTCCCATCCTGGAGAACCAG
ACAGTGACACTAGTCTGCAACACACCCAATGAGGCACCCAGTGATCTCCGCTACAGCTGGTACAAGAA
CCATGTCCTGCTGGAGGATGCCCACTCCCATACCCTCCGGCTGCACTTGGCCACTAGGGCTGATACTG
GCTTCTACTTCTGTGAGGTGCAGAACGTCCATGGCAGCGAGCGCTCGGGCCCTGTCAGCGTGGTAGTC
AACCACCCGCCTCTCACTCCAGTCCTGACAGCCTTCCTGGAGACCCAGGCGGGACTTGTGGGCATCCT
TCACTGCTCTGTGGTCAGTGAGCCCCTGGCCACACTGGTGCTGTCACATGGGGGTCATATCCTGGCCT
CCACCTCCGGGGACAGTGATCACAGCCCACGCTTCAGTGGTACCTCTGGTCCCAACTCCCTGCGCCTG
GAGATCCGAGACCTGGAGGAAACTGACAGTGGGGAGTACAAGTGCTCAGCCACCAACTCCCTTGGAAA
TGCAACCTCCACCCTGGACTTCCATGCCAATGCCGCCCGTCTCCTCATCAGCCCGGCAGCCGAGGTGG
TGGAAGGACAGGCAGTGACACTGAGCTGCAGAAGCGGCCTAAGCCCCACACCTGATGCCCGCTTCTCC
TGGTACCTGAATGGAGCCCTGCTTCACGAGGGTCCCGGCAGCAGCCTCCTGCTCCCCGCGGCCTCCAG
CACTGACGCCGGCTCATACCACTGCCGGGCCCGGGACGGCCACAGTGCCAGTGGCCCCTCTTCGCCAG
GGGGCCGGGGCTGGACGGCGAGGCCTCCTTTTGTGCCGTGTGGACAGCGACCCCCCCGCCAGGCTGCA
GCTGCTCCACAAGGACCGTGTTGTGGCCACTTCCCTGCCATCAGGGGGTGGCTGCAGCACCTGTGGGG
GCTGTTCCCCACGCATGAAGGTCACCAAAGCCCCCAACTTGCTGCGTGTGGAGATTCACAACCCTTTG
CTGGAAGAGGAGGGCTTGTACCTCTGTGAGGCCAGCAATGCCCTGGGCAACGCCTCCACCTCAGCCAC
CTTCAATGGCCAGGCCACTGTCCTGGCCATTGCACCATCACACACACTTCAGGAGGGCACAGAAGCCA
ACTTGACTTGCAACGTGAGCCGGGAAGCTGCTGGCAGCCCTGCTAACTTCTCCTGGTTCCGAAATGGG
GTGCTGTGGGCCCAGGGTCCCCTGGAGACCGTGACACTGCTGCCCGTGGCCAGAACTGATGCTGCCCT
TTACGCCTGCCGCATCCTGACTGAGGCTGGTGCCCAGCTCTCCACTCCCGTGCTCCTGAGTGTACTCT
ATCCCCCGGACCGTCCAAAGCTGTCAGCCCTCCTAGACATGGGCCAGGGCCACATGGCTCTGTTCATC
TGCACTGTGGACAGCCGCCCCCTGGCCTTGCTGGCCTTGTTCCATGGGGAGCACCTCCTGGCCACCAG
CCTGGGTCCCCAGGTCCCATCCCATGGTCGGTTCCAGGCTAAAGCTGAGGCCAACTCCCTGAAGTTAG
AGGTCCGAGAACTGGGCCTTGGGGACTCTGGCAGCTACCGCTGTGAGGCCACAAATGTTCTTGGATCA
TCCAACACCTCACTCTTCTTCCAGGTCCGAGGAGCCTGGGTCCAGGTGTCACCATCACCTGAGCTCCA
AGAGGGCCAGGCTGTGGTCCTGAGCTGCCAGGTACACACAGGAGTCCCAGAGGGGACCTCATATCGTT
GGTATCGGGATGGCCAGCCCCTCCAGGAGTCGACCTCGGCCACGCTCCGCTTTGCAGCCATAACTTTG
ACACAAGCTGGGGCCTATCATTGCCAAGCCCAGGCCCCAGGCTCAGCCACCACGAGCCTAGCTGCACC
CATCAGCCTCCACGTGTCCTATGCCCCACGCCACGTCACACTCACTACCCTGATGGACACAGGCCCTG
GACGACTGGGCCTCCTCCTGTGCCGTGTGGACAGTGACCCTCCGGCCCAGCTGCGGCTGCTCCACGGG
GATCGCCTTGTGGCCTCCACCCTACAAGGTGTGGGGGGACCCGAAGGCAGCTCTCCCAGGCTGCATGT
GGCTGTGGCCCCCAACACACTGCGTCTGGAGATCCACGGGGCTATGCTGGAGGATGAGGGTGTCTATA
TCTGTGAGGCCTCCAACACCCTGGGCCAGGCCTCGGCCTCAGCTGACTTCGACGCTCAAGCTGTGAAT
GTGCAGGTGTGGCCCGGGGCTACCGTGCGGGAGGGGCAGCTGGTGAACCTGACCTGCCTTGTGTGGAC
CACTCACCCGGCCCAGCTCACCTACACATGGTACCAGGATGGGCAGCAGCGCCTGGATGCCCACTCCA
GCACCCCGCCTCTCCAGACCTATCACCTTGGACGTCCTCTACGCGCCCCGCAACCTGCGCCTGACCTA
CCTCCTGGAGAGCCATGGCGGGCAGCTGGCCCTGGTACTGTGCACTGTGGACAGCCGCCCGCCCGCCC
AGCTGGCCCTCAGCCACGCCGGTCGCCTCTTGGCCTCCTCGACAGCAGCCTCTGTCCCCAACACCCTG
CGCCTGGAGCTGCGAGGGCCACAGCCCAGGGATGAGGGTTTCTACAGCTGCTCTGCCCGCAGCCCTCT
GGGCCAGGCCAACACGTCCCTGGAGCTGCGGCTGGAGGGTGTGCGGGTGATCCTGGCTCCGGAGGCTG
CCGTGCCTGAAGGTGCCCCCATCACAGTGACCTGTGCGGACCCTGCTGCCCACGCACCCACACTCTAT
ACTTGGTACCACAACGGTCGTTGGCTGCAGGAGGGTCCAGCTGCCTCACTCTCATTCCTGGTGGCCAC
GCGGGCTCATGCAGGCGCCTACTCTTGCCAGGCCCAGGATGCCCAGGGCACCCGCAGCTCCCGTCCTG
CTGCCCTGCAAGTCCTCTATGCCCCTCAGGACACTGTCCTGTCCTCCTTCCGGGACTCCAGGGCCAGA
TCCATGGCTGTGATACAGTGCACTGTGGACAGTGAGCCACCTGCTGAGCTGGCCCTATCTCATGATGG
CAAGGTGCTGGCCACGAGCAGCGGGGTCCACAGCTTGGCATCAGGGACAGGCCATGTCCAGGTGGCCC
GAAACGCCCTACGGCTGCAGGTGCAAGATGTGCCTGCAGGTGATGACACCTATGTTTGCACAGCCCAA
AACTTGCTGGGCTCAATCAGCACCATCGGGCGGTTGCAGGTAGAAGGTGCACGCGTGGTGGCAGAGCC
TGGGCAACTCCACCTTTGCATGGTTCTGGAATGACCGGCGGCTGCACGCGGAGCCTGTGCCCACTCTC
GCCTTCACCCACGTGGCTCGTGCTCAAGCTGGGATGTACCACTGCCTGGCTGAGCTCCCCACTGGGGC
TGCTGCCTCTGCTCCAGTCATGCTCCGTGTGCTCTACCCTCCCAAGACGCCCACCATGATGGTCTTCG
TGGAGCCTGAGGGTGGCCTCCGGGGCATCCTGGATTGCCGAGTGGACAGCGAGCCGCTCGCCAGCCTG
ACTCTCCACCTTGGCAGTCGACTGGTGGCCTCCAGTCAGCCCCAGGGTGCTCCTGCAGAGCCACACAT
CCATGTCCTGGCTTCCCCCAATGCCCTGAGGGTGGACATCGAGGCGCTGAGGCCCAGCGACCAAGGGG
AATACATCTGTTCTGCCTCAAATGTCCTGGGCTCTGCCTCTACCTCCACCTACTTTGGGGTCAGAGCC
CTGCACCGCCTGCATCAGTTCCAGCAGCTGCTCTGGGTCCTGGGACTGCTGGTGGGCCTCCTGCTCCT
GCTGTTGGGCCTGGGGGCCTGCTACACCTGGAGAAGGAGGCGTGTTTGTAAGCAGAGCATGGGCGAGA
ATTCGGTGGAGATGGCTTTTCAGAAAGAGACCACGCAGCTCATTGATCCTGATGCAGCCACATGTGAG
ACCTCAACCTGTGCCCCACCCCTGGGCTGA

| | | | |
|---|---|---|---|
| NOV14g, CG51821-04 | | | |
| Protein Sequence | SEQ ID NO: 410 | 1709 aa | Mw at 182651.9kD |

MGFLPKLLLLASFFPAGQASWGVSSPQDVQGVKGSCLLIPCIFSFPADVEVPDGITAIWYYDYSGQRQ
VVSHSADPKLVEARFRGRTEFMGNPEHRVCNLLLKDLQPEDSGSYNFRFEISEVNRWSDVKGTLVTVT
EEPRVPTIASPVELLEGTEVDFNCSTPYVCLQEQVRLQWQGQDPARSVTFNSQKFEPTGVGHLETLHM
AMSWQDHGRILRCQLSVANHRAQSEIHLQVKYAPKGVKILLSPSGRNILPGELVTLTCQVNSSYPAVS
SIKWLKDGVRLQTKTGVLHLPQAAWSDAGVYTCQAENGVGSLVSPPISLHIFMAEVQVSPAGPILENQ
TVTLVCNTPNEAPSDLRYSWYKNHVLLEDAHSHTLRLHLATRADTGFYFCEVQNVHGSERSGPVSVVV
NHPPLTPVLTAFLETQAGLVGILHCSVTSEPLATLVLSHGGHILASTSGDSDHSPRFSGTSGPNSLRL
EIRDLEETDSGEYKCSATNSLGNATSTLDFHANAARLLISPAAEVVEGQAVTLSCRSGLSPTPDARFS
WYLNGALLHEGPGSSLLLPAASSTDAGSYHCRARDGHSASGPSSPAVLTVLYPPRQPTFTTRLDLDAA
GAGAGRRGLLLCRVDSDPPAPLQLLHKDRVVATSLPSGGGCSTCGGCSPRNKVTKAPNLLRVEIHNPL
LEEEGLYLCEASNALGNASTSATFNGQATVLAIAPSHTLQEGTEANLTCNVSREAAGSPANFSWFRNG
VLWAQGPLETVTLLPVARTDAALYACRILTEAGAQLSTPVLLSVLYPPDRPKLSALLDMGQGHMALFI
CTVDSRPLALLALFHGEHLLATSLGPQVPSHGRFQAKAEANSLKLEVRELGLGDSGSYRCEATNVLGS
SNTSLFFQVRGAWVQVSPSPELQEGQAVVLSCQVHTGVPEGTSYRWYRDGQPLQESTSATLRFAAITL
TQAGAYHCQAQAPGSATTSLAAPISLHVSYAPRHVTLTTLMDTGPGRLGLLLCRVDSDPPAQLRLLHG
DRLVASTLQGVGGPEGSSPRLHVAVAPNTLRLEIHGAMLEDEGVYICEASNTLGQASASADFDAQAVN
VQVWPGATVREGQLVNLTCLVWTTHPAQLTYTWYQDGQQRLDAHSIPLPNVTVRDATSYRCGVGPPGR
APRLSRPITLDVLYAPRNLRLTYLLESHGGQLALVLCTVDSRPPAQLALSHAGRLLASSTAASVPNTL
RLELRGPQPRDEGFYSCSARSPLGQANTSLELRLEGVRVILAPEAAVPEGAPMMVTCADPAAHAPTLY
TWYHNGRWLQEGPAASLSFLVATRAHAGAYSCQAQDAQGTRSSRPAALQVLYAPQDTVLSSFRDSPAR
SMAVIQCTVDSEPPAELALSHDGKVLATSSGVHSLASGTGHVQVARNALRLQVQDVPAGDDTYVCTAQ
NLLGSISTIGRLQVEGARVVAEPGLDVPEGAALNLSCRLLGGPGPVGNSTFAWFWNDRRLHAEPVPTL
AFTHVARAQAGMYHCLAELPTGAAASAPVMLRVLYPPKTPTMMVFVEPEGGLRGILDCRVDSEPLASL
TLHLGSRLVASSQPQGAPAEPHIHVLASPNALRVDIEALRPSDQGEYICSASNVLGSASTSTYFGVRA
LHRLHQFQQLLWVLGLLVGLLLLLLGLGACYTWRRRRVCKQSMGENSVEMAFQKETTQLIDPDAATCE
TSTCAPPLG

| | | |
|---|---|---|
| NOV14h, | | |
| SNP1 3382490 | SEQ ID NO: 411 | 5103 bp |
| of CG51821-01, | ORF Start: ATG at 1 | ORF Stop: TGA at 5101 |
| DNA Sequence | SNP Pos: 4056 | SNP Change: C to T |

ATGGGCTTCTTGCCCAAGCTTCTCCTCCTGGCCTCAGCCGTTCTTCCCCCAGGCCAGGCCTCATGGGG
CGTCTCCAGTCCCCAGGACGTGCAGGGTGTGAAGGGGTCTTGCCTGCTTATCCCCTGCATCTTCAGCT
TCCCTGCCGACGTGGAGGTGCCCGACGGCATCACGGCCATCTGGTACTACGACTACTCGGGCCAGCGG
CAGGTGGTGAGCCACTCGGCGGACCCCAAGCTGGTGGAGGCCCGCTTCCGCGGCCGCACCGAGTTCAT
GGGGAACCCCGAGCACAGGGTGTGCAACCTGCTGCTGAAGGACCTGCAGCCCGAGGACTCTGGTTCCT
ACAACTTCCGCTTCGAGATCAGTGAGGTCAACCGCTGGTCAGATGTGAAAGGCACCTTGGTCACAGTA
ACAGGTGATCCCAGGGTGCCCACCATTGCCTCCCCGGTGGAGCTTCTCGAGGGCACAGAGGTGGACTT
CAACTGCTCCACTCCCTACGTATGCCTGCAGGAGCAGGTCAGACTGCAGTGGCAAGGCCAGGACCCTG
CTCGCTCTGTCACCTTCAACAGCCAGAAGTTTGAGCCCACCGGCGTCGGCCACCTGGAGACCCTCCAC
ATGGCCATGTCCTGGCAGGACCACGGCCGGATCCTGCGCTGCCAGCTCTCCGTGGCCAATCACAGGGC
TCAGAGCGAGATTCACCTCCAAGTGAAGTGTGCCCCCAAGGGTGTGAAGATCCTCCTCAGCCCCTCGG
GGAGGAACATCCTTCCAGGTGAGCTGGTCACACTCACCTGCCAGGTGAACAGCAGCTACCCTGCAGTC
GGCAGCCTGGAGCGATGCTGGCGTCTACACCTGCCAAGCTGAGAACGGCGTGGGCTCTTTGGTCTCAC
CCCCCATCAGCCTCCACATCTTCGTGGCTGAGGTCCAGGTGAGCCCAGCAGGTCCCATCCTGGAGAAC
CAGACAGTGACACTAGTCTGCAACACACCCAATGAGGCACCCAGTGATCTCCGCTACAGCTGGTACAA
GAACCATGTCCTGCTGGAGGATGCCCACTCCCATACCCTCCGGCTGCACTTGGCCACTAGGGCTGATA
CTGGCTTCTACTTCTGTGAGGTGCAGAACGTCCATGGCAGCGAGCGCTCGGGCCCTGTCAGCGTGGTA
GTCACAGACCCGCCTCTCACTCCAGTCCTGACAGCCTTCCTGGAGACCCAGGCGGGACTTGTGGGCAT
CCTTCACTGCTCTGTGGTCAGTGAGCCCCTGGCCACACTGGTGCTGTCACATGGGGGTCATATCCTGG
CCTCCACCTCCGGGGACAGTGATCACAGCCCACGCTTCAGTGGTACCTCTGGTCCCAACTCCCTGCGC
CTGGAGATCCGAGACCTGGAGGAAACTGACAGTGGGGAGTACAAGTGCTCAGCCACCAACTCCCTTGG
AAATGCAACCTCCACCCTGGACTTCCATGCCAATGTCGCCCGTCTCCTCATCAGCCCGGCAGCCGAGG
TGGTGGAAGGACAGGCAGTGACACTGAGCTGCAGAAGCGGCCTAAGCCCCACACCTGATGCCCGCTTC
TCCTGGTACCTGAATGGAGCCCTGCTTCACGAGGGTCCCGGCAGCAGCCTCCTGCTCCCCGCGGCCTC
CAGCACTGACGCCGGCTCATACCACTGCCGGGCCCGGGACGGCCACAGTGCCAGTGGCCCCTCTTCGC
CAGCTGTTCTCACTGTGCTCTGTGAGCAGCCACCACGACAACCAACATTCACCACCAGGCTGGACCTT
GATGCCGCTGGGGCCGGGGCTGGACGGCGAGGCCTCCTTTTGTGCCGTGTGGACAGCGACCCCCCCGC
CAGGCTGCAGCTGCTCCACAAGGACCGTGTTGTGGCCACTTCCCTGCCATCAGGGGGTGGCTGCAGCA
CCTGTGGGGGCTGTTCCCCACGCATGAAGGTCACCAAAGCCCCCAACTTGCTGCGTGTGGAGATTCAC
AACCCTTTGCTGGAAGAGGAGGGCTTGTACCTCTGTGAGGCCAGCAATGCCCTGGGCAACGCCTCCAC
CTCAGCCACCTTCAATGGCCAGGCCACTGTCCTGGCCATTGCACCATCACACACACTTCAGGAGGGCA
CAGAAGCCAACTTGACTTGCAACGTGAGCCGGGAAGCTGCTGGCAGCCCTGCTAACTTCTCCTGGTTC
CGAAATGGGGTGCTGTGGGCCCAGGGTCCCCTGGAGACCGTGACACTGCTGCCCGTGGCCAGAACTGA
TGCTGCCCTTTACGCCTGCCGCATCCTGACTGAGGCTGGTGCCCAGCTCTCCACTCCCGTGCTCCTGA
GTGTACTCTATCCCCCGGACCGTCCAAAGCTGTCAGCCCTCCTAGACATGGGCCAGGGCCACATGGCT
CTGTTCATCTGCACTGTGGACAGCCGCCCCCTGGCCTTGCTGGCCTTGTTCCATGGGGAGCACCTCCT
GGCCACCAGCCTGGGTCCCCAGGTCCCATCCCATGGTCGGTTCCAGGCTAAAGCTGAGGCCAACTCCC
TGAAGTTAGAGGTCCGAGAACTGGGCCTTGGGGACTCTGGCAGCTACCGCTGTGAGGCCACAAATGTT
CTTGGATCATCCAACACCTCACTCTTCTTCCAGGTCCGAGGTGCCTGGGTCCAGGTGTCACCATCACC
TGAGCTCCAAGAGGGCCAGGCTGTGGTCCTGAGCTGCCAGGTACACACAGGAGTCCCAGAGGGGACCT
CATATCGTTGGTATCGGGATGGCCAGCCCCTCCAGGAGTCGACCTCGGCCACGCTCCGCTTTGCAGCC
ATAACTTTGACACAAGCTGGGGCCTATCATTGCCAAGCCCAGGCCCCAGGCTCAGCCACCACGAGCCT
AGCTGCACCCATCAGCCTCCACGTGTCCTGTAAGGATGCCCCACGCCACGTCACACTCACTACCCTGA
TGGACACAGGCCCTGGACGACTGGGCCTCCTCCTGTGCCGTGTGGACAGTGACCCTCCGGCCCAGCTG
CGGCTGCTCCACGGGGATCGCCTTGTGGCCTCCACCCTACAAGGTGTGGGGGGACCCGAAGGCAGCTC
CCCACGGCTGCATGTGGCTGTGGCCCCCAACACACTGCGTCTGGAGATCCACGGGGCTATGCTGGAGG
ATGAGGGTGTCTATATCTGTGAGGCCTCCAACACCCTGGGCCAGGCCTCGGCCTCAGCTGACTTCGAC
CCTGCCTTGTGTGGACCACTCACCCGGCCCAGCTCACCTACACATGGTACCAGGATGGGCAGCAGCGC
CTGGATGCCCACTCCATCCCCCTGCCCAACGTCACAGTCAGGGATGCCACCTCCTACCGCTGCGGTGT
GGGCCCCCCTGGTCGGGCACCCCGCCTCTCCAGACCTATCACCTTGGACGTCCTCACGCGCCCCGCAA
CCTGCGCCTGACCTACCTCCTGGAGAGCCATGGCGGGCAGCTGGCCCTGGTACTGTGCACTGTGGACA
GCCGCCCGCCCGCCCAGCTGGCCCTCAGCCACGCCGGTCGCCTCTTGGCCTCCTCGACAGCAGCCTCT
GTCCCCAACACCCTGCGCCTGGAGCTGCGAGGGCCACAGCCCAGGGATGAGGGTTTCTACAGCTGCTC
TGCCCGCAGCCCTCTGGGCCAGGCCAACACGTCCCTGGAGCTGCGGCTGGAGGTGCGGGTGATCCTGG
CTCCGGAGGCTGCCGTGCCTGAAGGTGCCCCCATCACAGTGACCTGTGCGGACCCTGCTGCCCACGCA
CCCACACTCTATACTTGGTACCACAACGGTCGTTGGCTGCAGGAGGGTCCAGCTGCCTCACTCTCATT
CCTGGTGGCCACGCGGGCTCATGCAGGCGCCTACTCTTGCCAGGCCCAGGATGCCCAGGGCACCCGCA
GCTCCCGTCCTGCTGCCCTGCAAGTCCTCTGTGCCCCTCAGGATGCTGTCCTGTCCTCCTTCCGGGAC
TCCAGGGCCAGATCCATGGCTGTGATACAGTGCACTGTGGACAGTGAGCCACCTGCTGAGCTGGCCCT
ATCTCATGATGGCAAGGTGCTGGCCACGAGCAGCGGGGTCCACAGCTTGGCATCAGGGACAGGCCATG
TCCAGGTGGCCCGAAACGCCCTACGGCTGCAGGTGCAAGATGTGCCTGCAGGTGATGACACCTATGTT
TGCACAGCCCAAAACTTGCTGGGCTCAATCAGCACCATCGGGCGGTTGCAGGTAGAAGGTGAGTGGCG
GTGGCCCTGGGCCTGTGGGCAACTCCACCTTTGCATGGTTCTGGAATGACCGGCGGCTGCACGCGGAG
CCTGTGCCCACTCTCGCCTTCACCCACGTGGCTCGTGCTCAAGCTGGGATGTACCACTGCCTGGCTGA
GCTCCCCACTGGGGCTGCTGCCTCTGCTCCAGTCATGCTCCGTGTGCTCTACCCTCCCAAGACGCCCA
CCATGATGGTCTTCGTGGAGCCTGAGGGTGGCCTCCGGGGCATCCTGGATTGCCGAGTGGACAGCGAG
CCGCTCGCCAGCCTGACTCTCCACCTTGGCAGTCGACTGGTGGCCTCCAGTCAGCCCCAGGGTGCTCC
TGCAGAGCCACACATCCATGTCCTGGCTTCCCCCAATGCCCTGAGGGTGGACATCGAGGCGCTGAGGC
TTTGGGGTCAGAGCCCTGCACCGCCTGCATCAGTTCCAGCAGCTGCTCTGGGTCCTGGGACTGCTGGT
GGGCCTCCTGCTCCTGCTGTTGGGCCTGGGGGCCTGCTACACCTGGAGAAGGAGGCGTGTTTGTAAGC
AGAGCATGGGCGAGAATCCAGGGCGGGCATCGTCTTTCCATTTACTGCCTCTAGCTGGGTCTTCAAGG
TGA

| | | | |
|---|---|---|---|
| NOV14h, | | | |
| SNP13382490 of | | | |
| CG51821-01, | SEQ ID NO: 412 | | MW at 180833.0kD |
| Protein Sequence | SNP Pos: 1352 | 1700 aa | SNP Change: Asp to Asp |

MGFLPKLLLLASAVLPPGQASWGVSSPQDVQGVKGSCLLIPCIFSFPADVEVPDGITAIWYYDYSGQR
QVVSHSADPKLVEARFRGRTEFMGNPEHRVCNLLLKDLQPEDSGSYNFRFEISEVNRWSDVKGTLVTV
TGDPRVPTIASPVELLEGTEVDFNCSTPYVCLQEQVRLQWQGQDPARSVTFNSQKFEPTGVGHLETLH
MAMSWQDHGRILRCQLSVANHRAQSEIHLQVKCAPKGVKILLSPSGRNILPGELVTLTCQVNSSYPAV
SSIKWLKDGVRLQTKTGVLHLPQAAWSDAGVYTCQAENGVGSLVSPPISLHIFVAEVQVSPAGPILEN
QTVTLVCNTPNEAPSDLRYSWYKNHVLLEDAHSHTLRLHLATRADTGFYFCEVQNVHGSERSGPVSVV
VTDPPLTPVLTAFLETQAGLVGILHCSVVSEPLATLVLSHGGHILASTSGDSDHSPRFSGTSGPNSLR
LEIRDLEETDSGEYKCSATNSLGNATSTLDFHANVARLLISPAAEVVEGQAVTLSCRSGLSPTPDARF
SWYLNGALLHEGPGSSLLLPAASSTDAGSYHCRARDGHSASGPSSPAVLTVLCEQPPRQPTFTTRLDL
DAAGAGAGPRGLLLCRVDSDPPARLQLLHKDRVVATSLPSGGGCSTCGGCSPRMKVTKAPNLLRVEIH
NPLLEEEGLYLCEASNALGNASTSATFNGQATVLAIAPSHTLQEGTEANLTCNVSREAAGSPANFSWF
RNGVLWAQGPLETVTLLPVARTDAALYACRILTEAGAQLSTPVLLSVLYPPDRPKLSALLDMGQGHMA
LFICTVDSRPLALLALFHGEHLLATSLGPQVPSHGRFQAKAEANSLKLEVRELGLGDSGSYRCEATNV
LGSSNTSLFFQVRGAWVQVSPSPELQEGQAVVLSCQVHTGVPEGTSYRWYRDGQPLQESTSATLRFAA
ITLTQAGAYHCQAQAPGSATTSLAAPISLHVSCKDAPRHVTLTTLMDTGPGRLGLLLCRVDSDPPAQL
RLLHGDRLVASTLQGVGGPEGSSPRLHVAVAPNTLRLEIHGAMLEDEGVYICEASNTLGQASASADFD
GPPWSGTPPLQTYHLGRPHAPRNLRLTYLLESHGGQLALVLCTVDSRPPAQLALSHAGRLLASSTAAS
VPNTLRLELRGPQPRDEGFYSCSARSPLGQANTSLELRLEVRVILAPEAAVPEGAPITVTCADPAAHA
PTLYTWYHNGRWLQEGPAASLSFLVATRAHAGAYSCQAQDAQGTRSSRPAALQVLCAPQDAVLSSFRD
SRARSMAVIQCTVDSEPPAELALSHDGKVLATSSGVHSLASGTGHVQVARNALRLQVQDVPAGDDTYV
CTAQNLLGSISTIGRLQVEGEWRVVAEPGLDVPEGAALNLSCRLLGGPGPVGNSTFAWFWNDRRLHAE
PVPTLAFTHVARAQAGMYHCLAELPTGAAASAPVMLRVLYPPKTPTMMVFVEPEGGLRGILDCRVDSE
PLASLTLHLGSRLVASSQPQGAPAEPHIHVLASPNALRVDIEALRPSDQGEYICSASNVLGPASTSTY
FGVRALHRLHQFQQLLWVLGLLVGLLLLLLGLGACYTWRRRRVCKQSMGENPGPASSFHLLPLAGSSR

| | | |
|---|---|---|
| NOV14i, | | |
| SNP1 3382489 of | SEQ ID NO: 413 | 5103 bp |
| CG51821-01, | ORF Start: ATG at 1 | ORF Stop: TGA at 5101 |
| DNA Sequence | SNP Pos: 4245 | SNP Change: G to A |

ATGGGCTTCTTGCCCAAGCTTCTCCTCCTGGCCTCAGCCGTTCTTCCCCCAGGCCAGGCCTCATGGGG
CGTCTCCAGTCCCCAGGACGTGCAGGGTGTGAAGGGGTCTTGCCTGCTTATCCCCTGCATCTTCAGCT
TCCCTGCCGACGTGGAGGTGCCCGACGGCATCACGGCCATCTGGTACTACGACTACTCGGGCCAGCGG
CAGGTGGTGAGCCACTCGGCGGACCCCAAGCTGGTGGAGGCCCGCTTCCGCGGCCGCACCGAGTTCAT
GGGGAACCCCGAGCACAGGGTGTGCAACCTGCTGCTGAAGGACCTGCAGCCCGAGGACTCTGGTTCCT
ACAACTTCCGCTTCGAGATCAGTGAGGTCAACCGCTGGTCAGATGTGAAAGGCACCTTGGTCACAGTA
ACAGGTGATCCCAGGGTGCCCACCATTGCCTCCCCGGTGGAGCTTCTCGAGGGCACAGAGGTGGACTT
CAACTGCTCCACTCCCTACGTATGCCTGCAGGAGCAGGTCAGACTGCAGTGGCAAGGCCAGGACCCTG
CTCGCTCTGTCACCTTCAACAGCCAGAAGTTTGAGCCCACCGGCGTCGGCCACCTGGAGACCCTCCAC
ATGGCCATGTCCTGGCAGGACCACGGCCGGATCCTGCGCTGCCAGCTCTCCGTGGCCAATCACAGGGC
TCAGAGCGAGATTCACCTCCAAGTGAAGTGTGCCCCCAAGGGTGTGAAGATCCTCCTCAGCCCCTCGG
GGAGGAACATCCTTCCAGGTGAGCTGGTCACACTCACCTGCCAGGTGAACAGCAGCTACCCTGCAGTC
AGTTCCATTAAGTGGCTCAAGGATGGGGTACGCCTCCAAACCAAGACTGGTGTGCTGCACCTGCCCCA
GGCAGCCTGGAGCGATGCTGGCGTCTACACCTGCCAAGCTGAGAACGGCGTGGGCTCTTTGGTCTCAC
CCCCCATCAGCCTCCACATCTTCGTGGCTGAGGTCCAGGTGAGCCCAGCAGGTCCCATCCTGGAGAAC
CAGACAGTGACACTAGTCTGCAACACACCCAATGAGGCACCCAGTGATCTCCGCTACAGCTGGTACAA
GAACCATGTCCTGCTGGAGGATGCCCACTCCCATACCCTCCGGCTGCACTTGGCCACTAGGGCTGATA
CTGGCTTCTACTTCTGTGAGGTGCAGAACGTCCATGGCAGCGAGCGCTCGGGCCCTGTCAGCGTGGTA
GTCACAGACCCGCCTCTCACTCCAGTCCTGACAGCCTTCCTGGAGACCCAGGCGGGACTTGTGGGCAT
CCTTCACTGCTCTGTGGTCAGTGAGCCCCTGGCCACACTGGTGCTGTCACATGGGGGTCATATCCTGG
CCTCCACCTCCGGGGACAGTGATCACAGCCCACGCTTCAGTGGTACCTCTGGTCCCAACTCCCTGCGC
CTGGAGATCCGAGACCTGGAGGAAACTGACAGTGGGGAGTACAAGTGCTCAGCCACCAACTCCCTTGG
AAATGCAACCTCCACCCTGGACTTCCATGCCAATGTCGCCCGTCTCCTCATCAGCCCGGCAGCCGAGG
TGGTGGAAGGACAGGCAGTGACACTGAGCTGCAGAAGCGGCCTAAGCCCCACACCTGATGCCCGCTTC
TCCTGGTACCTGAATGGAGCCCTGCTTCACGAGGGTCCCGGCAGCAGCCTCCTGCTCCCCGCGGCCTC
CAGCACTGACGCCGGCTCATACCACTGCCGGGCCCGGGACGGCCACAGTGCCAGTGGCCCCTCTTCGC
CAGCTGTTCTCACTGTGCTCTGTGAGCAGCCACCACGACAACCAACATTCACCACCAGGCTGGACCTT
CAGGCTGCAGCTGCTCCACAAGGACCGTGTTGTGGCCACTTCCCTGCCATCAGGGGGTGGCTGCAGCA
CCTGTGGGGGCTGTTCCCCACGCATGAAGGTCACCAAAGCCCCCAACTTGCTGCGTGTGGAGATTCAC
AACCCTTTGCTGGAAGAGGAGGGCTTGTACCTCTGTGAGGCCAGCAATGCCCTGGGCAACGCCTCCAC
CTCAGCCACCTTCAATGGCCAGGCCACTGTCCTGGCCATTGCACCATCACACACACTTCAGGAGGGCA
CAGAAGCCAACTTGACTTGCAACGTGAGCCGGGAAGCTGCTGGCAGCCCTGCTAACTTCTCCTGGTTC
CGAAATGGGGTGCTGTGGGCCCAGGGTCCCCTGGAGACCGTGACACTGCTGCCCGTGGCCAGAACTGA
TGCTGCCCTTTACGCCTGCCGCATCCTGACTGAGGCTGGTGCCCAGCTCTCCACTCCCGTGCTCCTGA
GTGTACTCTATCCCCCGGACCGTCCAAAGCTGTCAGCCCTCCTAGACATGGGCCAGGGCCACATGGCT
CTGTTCATCTGCACTGTGGACAGCCGCCCCCTGGCCTTGCTGGCCTTGTTCCATGGGGAGCACCTCCT
GGCCACCAGCCTGGGTCCCCAGGTCCCATCCCATGGTCGGTTCCAGGCTAAAGCTGAGGCCAACTCCC
TGAAGTTAGAGGTCCGAGAACTGGGCCTTGGGGACTCTGGCAGCTACCGCTGTGAGGCCACAAATGTT
CTTGGATCATCCAACACCTCACTCTTCTTCCAGGTCCGAGGTGCCTGGGTCCAGGTGTCACCATCACC
TGAGCTCCAAGAGGGCCAGGCTGTGGTCCTGAGCTGCCAGGTACACACAGGAGTCCCAGAGGGGACCT
CATATCGTTGGTATCGGGATGGCCAGCCCCTCCAGGAGTCGACCTCGGCCACGCTCCGCTTTGCAGCC
ATACTaTTGACACAAGCTGGGGCCTATCATTGCCAAGCCCAGGCCCCAGGCTCAGCCACCACGAGCCT
AGCTGCACCCATCAGCCTCCACGTGTCCTGTAAGGATGCCCCACGCCACGTCACACTCACTACCCTGA
TGGACACAGGCCCTGGACGACTGGGCCTCCTCCTGTGCCGTGTGGACAGTGACCCTCCGGCCCAGCTG
CGGCTGCTCCACGGGGATCGCCTTGTGGCCTCCACCCTACAAGGTGTGGGGGGACCCGAAGGCAGCTC
TCCCAGGCTGCATGTGGCTGTGGCCCCCAACACACTGCGTCTGGAGATCCACGGGGCTATGCTGGAGG
ATGAGGGTGTCTATATCTGTGAGGCCTCCAACACCCTGGGCCAGGCCTCGGCCTCAGCTGACTTCGAC
GCTCAAAGCTGTGAATGTGCAGGTGTGGCCCGGGGCTACCGTGCGGGAGGGGCAGCTGGTGAACCTGA
CCTGGATGCCCACTCCATCCCCCTGCCAACGTCACAGTCAGGGATGCCACCTCCTACCGCTGCGGTGT
GGGCCCCCCTGGTCGGGCACCCCGCCTCTCCAGACCTATCACCTTGGACGTCCTCACGCGCCCCGCAA
CCTGCGCCTGACCTACCTCCTGGAGAGCCATGGCGGGCAGCTGGCCCTGGTACTGTGCACTGTGGACA
GCCGCCCGCCCGCCCAGCTGGCCCTCAGCCACGCCGGTCGCCTCTTGGCCTCCTCGACAGCAGCCTCT
GTCCCCAACACCCTGCGCCTGGAGCTGCGAGGGCCACAGCCCAGGGATGAGGGTTTCTACAGCTGCTC
TGCCCGCAGCCCTCTGGGCCAGGCCAACACGTCCCTGGAGCTGCGGCTGGAGGTGCGGGTGATCCTGG
CTCCGGAGGCTGCCGTGCCTGAAGGTGCCCCCATCACAGTGACCTGTGCGGACCCTGCTGCCCACGCA
CCCACACTCTATACTTGGTACCACAACGGTCGTTGGCTGCAGGAGGGTCCAGCTGCCTCACTCTCATT
CCTGGTGGCCACGCGGGCTCATGCAGGCGCCTACTCTTGCCAGGCCCAGGATGCCCAGGGCACCCGCA
GCTCCCGTCCTGCTGCCCTGCAAGTCCTCTGTGCCCCTCAGGACGCTGTCCTGTCCTCCTTCCGGGAC
TCCAGGGCCAGATCCATGGCTGTGATACAGTGCACTGTGGACAGTGAGCCACCTGCTGAGCTGGCCCT
ATCTCATGATGGCAAGGTGCTGGCCACGAGCAGCGGGGTCCACAGCTTGGCATCAGGGACAGGCCATG
TCCAGGTGGCCCGAAACGCCCTACGGCTACAGGTGCAAGATGTGCCTGCAGGTGATGACACCTATGTT
TGCACAGCCCAAAACTTGCTGGGCTCAATCAGCACCATCGGGCGGTTGCAGGTAGAAGGTGAGTGGCG
CGTGGTGGCAGAGCCTGGCCTGGACGTGCCTGAGGGCGCTGCCCTGAACCTCAGCTGCCGCCTCCTGG
GTGGCCCTGGGCCTGTGGGCAACTCCACCTTTGCATGGTTCTGGAATGACCGGCGGCTGCACGCGGAG
CCTGTGCCCACTCTCGCCTTCACCCACGTGGCTCGTGCTCAAGCTGGGATGTACCACTGCCTGGCTGA
GCTCCCCACTGGGGCTGCTGCCTCTGCTCCAGTCATGCTCCGTGTGCTCTACCCTCCCAAGACGCCCA
CCATGATGGTCTTCGTGGAGCCTGAGGGTGGCCTCCGGGGCATCCTGGATTGCCGAGTGGACAGCGAG
CCGCTCGCCAGCCTGACTCTCCACCTTGGCAGTCGACTGGTGGCCTCCAGTCAGCCCCAGGGTGCTCC
TGCAGAGCCACACATCCATGTCCTGGCTTCCCCCAATGCCCTGAGGGTGGACATCGAGGCGCTGAGGC
CCAGCGACCAAGGGGAATACATCTGTTCTGCCTCAAATGTCCTGGGCCCTGCCTCTACCTCCACCTAC
TTTGGGGTCAGAGCCCTGCACCGCCTGCATCAGTTCCAGCAGCTGCTCTGGGTCCTGGGACTGCTGGT
GGGCCTCCTGCTCCTGCTGTTGGGCCTGGGGGCCTGCTACACCTGGAGAAGGAGGCGTGTTTGTAAGC
AGAGCATGGGCGAGAATCCAGGGCGGGCATCGTCTTTCCATTTACTGCCTCTAGCTGGGTCTTCAAGG
TGA

| | | | |
|---|---|---|---|
| NOV14i, | | | |
| SNP1 3382489 of | | | |
| CG51821-01, | SEQ ID NO: 414 | | MW at 180833.0kD |
| Protein Sequence | SNP Pos: 1415 | 1700 aa | SNP Change: Leu to Leu |

MGFLPKLLLLASAVLPPGQASWGVSSPQDVQGVKGSCLLIPCIFSFPADVEVPDGITAIWYYDYSGQR
QVVSHSADPKLVEARFRGRTEFMGNPEHRVCNLLLKDLQPEDSGSYNFRFEISEVNRWSDVKGTLVTV
TGDPRVPTIASPVELLEGTEVDFNCSTPYVCLQEQVRLQWQGQDPARSVTFNSQKFEPTGVGHLETLH
MAMSWQDHGRILRCQLSVANHRAQSEIHLQVKCAPKGVKILLSPSGRNILPGELVTLTCQVNSSYPAV
SSIKWLKDGVRLQTKTGVLHLPQAAWSDAGVYTCQAENGVGSLVSPPISLHIFVAEVQVSPAGPILEN
QTVTLVCNTPNEAPSDLRYSWYKNHVLLEDAHSHTLRLHLATEADTGFYFCEVQNVHGSERSGPVSVV
VTDPPLTPVLTAFLETQAGLVGILHCSVVSEPLATLVLSHGGHILASTSGDSDHSPRFSGTSGPNSLR
LEIRDLEETDSGEYKCSATNSLGNATSTLDFHANVARLLISPAAEVVEGQAVTLSCRSGLSPTPDARF
SWYLNGALLHEGPGSSLLLPAASSTDAGSYHCRARDGHSASGPSSPATLTVLCEQPPRQPTFTTRLDL
DAAGAGAGRRGLLLCRVDSDPPARLQLLHKDRVVATSLPSGGGCSTCGGCSPRMKVTKAPNLLRVEIH
NPLLEEEGLYLCEASNALGNASTSATFNGQATVLAIAPSHTLQEGTEANLTCNVSREAAGSPANFSWF
RNGVLWAQGPLETVTLLPVARTDAALYACRILTEAGAQLSTPVLLSVLYPPDRPKLSALLDMGQGHMA
LFICTVDSRPLALLALFHGEHLLATSLGPQVPSHGRFQAKAEANSLKLEVRELGLGDSGSYRCEATNV
LGSSNTSLFFQVRGAWVQVSPSPELQEGQAVVLSCQVHTGVPEGTSYRWYRDGQPLQESTSATLRFAA
ITLTQAGAYHCQAQAPGSATTSLAAPISLHVSCKDAPRHVTLTTLMDTGPGRLGLLLCRVDSDPPAQL
RLLHGDRLVASTLQGVGGPEGSSPRLHVAVAPNTLRLEIHGAMLEDEGVYICEASNTLGQASASADFD
AQSCECAGVARGYRAGGAAGEPDLPCVDHSPGPAHLHMVPGWAAAPGCPLHPPAQRHSQGCHLLPLRC
GPPWSGTPPLQTYHLGRPHAPRNLRLTYLLESHGGQLALVLCTVDSRPPAQLALSHAGRLLASSTAAS
VPNTLRLELRGPQPRDEGFYSCSARSPLGQANTSLELRLEVRVILAPEAAVPEGAPITVTCADPAAHA
PTLYTWYHNGRWLQEGPAASLSFLVATRAHAGAYSCQAQDAQGTRSSRPAALQTLCAPQDAVLSSFRD
SRARSMAVIQCTVDSEPPAELALSHDGKVLATSSGVHSLASGTGHVQVARNALRLQVQDVPAGDDTYV
CTAQNLLGSISTIGRLQVEGEWRVVAEPGLDVPEGAALNLSCRLLGGPGPVGNSTFAWFWNDRRLHAE
PVPTLAFTHVARAQAGMYHCLAELPTGAAASAPVMLRVLYPPKTPTMMVFVEPEGGLRGILDCRVDSE
PLASLTLHLGSRLVASSQPQGAPAEPHIHVLASPNALRVDIEALRPSDQGEYICSASNVLGPASTSTY
FGVRALHRLHQFQQLLWVLGLLVGLLLLLLGLGACYTWRRRRVCKQSMGENPGRASSFHLLPLAGSSR

| | | |
|---|---|---|
| NOV14j, | | |
| SNP1 3377616 of | SEQ ID NO: 415 | 5103 bp |
| CG51821-01, | ORF Start: ATG at 1 | ORF Stop: TGA at 5101 |
| DNA Sequence SNP Pos: 4768 | SNP Change: C to T | |

ATGGGCTTCTTGCCCAAGCTTCTCCTCCTGGCCTCAGCCGTTCTTCCCCCAGGCCAGGCCTCATGGGG
CGTCTCCAGTCCCCAGGACGTGCAGGGTGTGAAGGGGTCTTGCCTGCTTATCCCCTGCATCTTCAGCT
TCCCTGCCGACGTGGAGGTGCCCGACGGCATCACGGCCATCTGGTACTACGACTACTCGGGCCAGCGG
GGGGAACCCCGAGCACAGGGTGTGCAACCTGCTGCTGAAGGACCTGCAGCCCGAGGACTCTGGTTCCT
ACAACTTCCGCTTCGAGATCAGTGAGGTCAACCGCTGGTCAGATGTGAAAGGCACCTTGGTCACAGTA
ACAGGTGATCCCAGGGTGCCCACCATTGCCTCCCCGGTGGAGCTTCTCGAGGGCACAGAGGTGGACTT
CAACTGCTCCACTCCCTACGTATGCCTGCAGGAGCAGGTCAGACTGCAGTGGCAAGGCCAGGACCCTG
CTCGCTCTGTCACCTTCAACAGCCAGAAGTTTGAGCCCACCGGCGTCGGCCACCTGGAGACCCTCCAC
TCAGAGCGAGATTCACCTCCAAGTGAAGTGTGCCCCCAAGGGTGTGAAGATCCTCCTCAGCCCCTCGG
GGAGGAACATCCTTCCAGGTGAGCTGGTCACACTCACCTGCCAGGTGAACAGCAGCTACCCTGCAGTC
AGTTCCATTAAGTGGCTCAAGGATGGGGTACGCCTCCAAACCAAGACTGGTGTGCTGCACCTGCCCCA
GGCAGCCTGGAGCGATGCTGGCGTCTACACCTGCCAAGCTGAGAACGGCGTGGGCTCTTTGGTCTCAC
CCCCCATCAGCCTCCACATCTTCGTGGCTGAGGTCCAGGTGAGCCCAGCAGGTCCCATCCTGGAGAAC
CAGACAGTGACACTAGTCTGCAACACACCCAATGAGGCACCCAGTGATCTCCGCTACAGCTGGTACAA
GAACCATGTCCTGCTGGAGGATGCCCACTCCCATACCCTCCGGCTGCACTTGGCCACTAGGGCTGATA
CTGGCTTCTACTTCTGTGAGGTGCAGAACGTCCATGGCAGCGAGCGCTCGGGCCCTGTCAGCGTGGTA
GTCACAGACCCGCCTCTCACTCCAGTCCTGACAGCCTTCCTGGAGACCCAGGCGGGACTTGTGGGCAT
CCTTCACTGCTCTGTGGTCAGTGAGCCCCTGGCCACACTGGTGCTGTCACATGGGGGTCATATCCTGG
CCTCCACCTCCGGGGACAGTGATCACAGCCCACGCTTCAGTGGTACCTCTGGTCCCAACTCCCTGCGC
CTGGAGATCCGAGACCTGGAGGAAACTGACAGTGGGGAGTACAAGTGCTCAGCCACCAACTCCCTTGG
AAATGCAACCTCCACCCTGGACTTCCATGCCAATGTCGCCCGTCTCCTCATCAGCCCGGCAGCCGAGG
TGGTGGAAGGACAGGCAGTGACACTGAGCTGCAGAAGCGGCCTAAGCCCCACACCTGATGCCCGCTTC
TCCTGGTACCTGAATGGAGCCCTGCTTCACGAGGGTCCCGGCAGCAGCCTCCTGCTCCCCGCGGCCTC
CAGCACTGACGCCGGCTCATACCACTGCCGGGCCCGGGACGGCCACAGTGCCAGTGGCCCCTCTTCGC
CAGCTGTTCTCACTGTGCTCTGTGAGCAGCCACCACGACAACCAACATTCACCACCAGGCTGGACCTT
GATGCCGCTGGGGCCGGGGCTGGACGGCGAGGCCTCCTTTTGTGCCGTGTGGACAGCGACCCCCCCGC
CAGGCTGCAGCTGCTCCACAAGGACCGTGTTGTGGCCACTTCCCTGCCATCAGGGGGTGGCTGCAGCA
CCTGTGGGGGCTGTTCCCCACGCATGAAGGTCACCAAAGCCCCCAACTTGCTGCGTGTGGAGATTCAC
AACCCTTTGCTGGAAGAGGAGGGCTTGTACCTCTGTGAGGCCAGCAATGCCCTGGGCAACGCCTCCAC
CTCAGCCACCTTCAATGGCCAGGCCACTGTCCTGGCCATTGCACCATCACACACACTTCAGGAGGGCA
CAGAAGCCAACTTGACTTGCAACGTGAGCCGGGAAGCTGCTGGCAGCCCTGCTAACTTCTCCTGGTTC
CGAAATGGGGTGCTGTGGGCCCAGGGTCCCCTGGAGACCGTGACACTGCTGCCCGTGGCCAGAACTGA
TGCTGCCCTTTACGCCTGCCGCATCCTGACTGAGGCTGGTGCCCAGCTCTCCACTCCCGTGCTCCTGA
CTGTTCATCTGCACTGTGGACAGCCGCCCCCTGGCCTTGCTGGCCTTGTTCCATGGGGAGCACCTCCT
GGCCACCAGCCTGGGTCCCCAGGTCCCATCCCATGGTCGGTTCCAGGCTAAAGCTGAGGCCAACTCCC
TGAAGTTAGAGGTCCGAGAACTGGGCCTTGGGGACTCTGGCAGCTACCGCTGTGAGGCCACAAATGTT
CTTGGATCATCCAACACCTCACTCTTCTTCCAGGTCCGAGGTGCCTGGGTCCAGGTGTCACCATCACC
TGAGCTCCAAGAGGGCCAGGCTGTGGTCCTGAGCTGCCAGGTACACACAGGAGTCCCAGAGGGGACCT
CATATCGTTGGTATCGGGATGGCCAGCCCCTCCAGGAGTCGACCTCGGCCACGCTCCGCTTTGCAGCC
AGCTGCACCCATCAGCCTCCACGTGTCCTGTAAGGATGCCCCACGCCACGTCACACTCACTACCCTGA
TGGACACAGGCCCTGGACGACTGGGCCTCCTCCTGTGCCGTGTGGACAGTGACCCTCCGGCCCAGCTG
TCCCAGGCTGCATGTGGCTGTGGCCCCCAACACACTGCGTCTGGAGATCCACGGGGCTATGCTGGAGG
GCTCAAAGCTGTGAATGTGCAGGTGTGGCCCGGGGCTACCGTGCGGGAGGGGCAGCTGGTGAACCTGA
CCTGCCTTGTGTGGACCACTCACCCGGCCCAGCTCACCTACACATGGTACCAGGATGGGCAGCAGCGC
CTGGATGCCCACTCCATCCCCCTGCCCAACGTCACAGTCAGGGATGCCACCTCCTACCGCTGCGGTGT
GGGCCCCCCTGGTCGGGCACCCCGCCTCTCCAGACCTATCACCTTGGACGTCCTCACGCGCCCCGCAA
CCTGCGCCTGACCTACCTCCTGGAGAGCCATGGCGGGCAGCTGGCCCTGGTACTGTGCACTGTGGACA
GCCGCCCGCCCGCCCAGCTGGCCCTCAGCCACGCCGGTCGCCTCTTGGCCTCCTCGACAGCAGCCTCT
GTCCCCAACACCCTGCGCCTGGAGCTGCGAGGGCCACAGCCCAGGGATGAGGGTTTCTACAGCTGCTC
TGCCCGCAGCCCTCTGGGCCAGGCCAACACGTCCCTGGAGCTGCGGCTGGAGGTGCGGGTGATCCTGG
CTCCGGAGGCTGCCGTGCCTGAAGGTGCCCCCATCACAGTGACCTGTGCGGACCCTGCTGCCCACGCA
CCCACACTCTATACTTGGTACCACAACGGTCGTTGGCTGCAGGAGGGTCCAGCTGCCTCACTCTCATT
CCTGGTGGCCACGCGGGCTCATGCAGGCGCCTACTCTTGCCAGGCCCAGGATGCCCAGGGCACCCGCA
GCTCCCGTCCTGCTGCCCTGCAAGTCCTCTGTGCCCCTCAGGACGCTGTCCTGTCCTCCTTCCGGGAC
TCCAGGGCCAGATCCATGGCTGTGATACAGTGCACTGTGGACAGTGAGCCACCTGCTGAGCTGGCCCT
ATCTCATGATGGCAAGGTGCTGGCCACGAGCAGCGGGGTCCACAGCTTGGCATCAGGGACAGGCCATG
TGCACAGCCCAAAACTTGCTGGGCTCAATCAGCACCATCGGGCGGTTGCAGGTAGAAGGTGAGTGGCG
CGTGGTGGCAGAGCCTGGCCTGGACGTGCCTGAGGGCGCTGCCCTGAACCTCAGCTGCCGCCTCCTGG
GTGGCCCTGGGCCTGTGGGCAACTCCACCTTTGCATGGTTCTGGAATGACCGGCGGCTGCACGCGGAG
CCTGTGCCCACTCTCGCCTTCACCCACGTGGCTCGTGCTCAAGCTGGGATGTACCACTGCCTGGCTGA
GCTCCCCACTGGGGCTGCTGCCTCTGCTCCAGTCATGCTCCGTGTGCTCTACCCTCCCAAGACGCCCA
CCGCTCGCCAGCCTGACTCTCCACCTTGGCAGTCGACTGGTGGCCTCCAGTCAGCCCCAGGGTGCTCC
TGCAGAGTCACACATCCATGTCCTGGCTTCCCCCAATGCCCTGAGGGTGGACATCGAGGCGCTGAGGC
CCAGCGACCAAGGGGAATACATCTGTTCTGCCTCAAATGTCCTGGGCCCTGCCTCTACCTCCACCTAC
TTTGGGGTCAGAGCCCTGCACCGCCTGCATCAGTTCCAGCAGCTGCTCTGGGTCCTGGGACTGCTGGT
GGGCCTCCTGCTCCTGCTGTTGGGCCTGGGGGCCTGCTACACCTGGAGAAGGAGGCGTGTTTGTAAGC
AGAGCATGGGCGAGAATCCAGGGCGGGCATCGTCTTTCCATTTACTGCCTCTAGCTGGGTCTTCAAGG
TGA

| | | | |
|---|---|---|---|
| NOV14j, | | | |
| SNP13377616 of | | | |
| CG51821-01, | SEQ ID NO: 416 | | MW at 180822.9kD |
| Protein Sequence | SNP Pos: 1590 | 1700 aa | SNP Change: Pro to Ser |

MGFLPKLLLLASAVLPPGQASWGVSSPQDVQGVKGSCLLIPCIFSFPADVEVPDGITAIWYYDYSGQR
QVVSHSADPKLVEARFRGRTEFMGNPEHRVCNLLLKDLQPEDSGSYNFRFEISEVNRWSDVKGTLVTV
TGDPRVPTIASPVELLEGTEVDFNCSTPYVCLQEQVRLQWQGQDPARSVTFNSQKFEPTGVGHLETLH
MAMSWQDHGRILRCQLSVANHRAQSEIHLQVKCAPKGVKILLSPSGPNILPGELVTLTCQVNSSYPAV
SSIKWLKDGVRLQTKTGVLHLPQAAWSDAGVYTCQAENGVGSLVSPPISLHIFVAEVQVSPAGPILEN
QTVTLVCNTPNEAPSDLRYSWYKNHVLLEDAHSHTLRLHLATRADTGFYFCEVQNVHGSERSGPVSVV
VTDPPLTPVLTAFLETQAGLVGILHCSVVSEPLATLVLSHGGHILASTSGDSDHSPRFSGTSGPNSLR
LEIRDLEETDSGEYKCSATNSLGNATSTLDFHANVARLLISPAAEVVEGQAVTLSCRSGLSPTPDARF
SWYLNGALLHEGPGSSLLLPAASSTDAGSYHCRARDGHSASGPSSPAVLTVLCEQPPRQPTFTTRLDL
DAAGAGAGRRGLLLCRVDSDPPARLQLLHKDRVVATSLPSGGGCSTCGGCSPRMKVTKAPNLLRVEIH
NPLLEEEGLYLCEASNALGNASTSATFNGQATVLAIAPSHTLQEGTEANLTCNVSREAAGSPANFSWF
RNGVLWAQGPLETVTLLPVARTDAALYACRILTEAGAQLSTPVLLSVLYPPDRPKLSALLDMGQGHMA
LFICTVDSRPLALLALFHGEHLLATSLGPQVPSHGRFQAKAEANSLKLEVRELGLGDSGSYRCEATNV
LGSSNTSLFFQVRGAWVQVSPSPELQEGQAVVLSCQVHTGVPEGTSYRWYRDGQPLQESTSATLRFAA
TLTQAGAYHCQAQAPGSATTSLAAPISLHVSCKDAPRHVTLTTLLMDTGPGRLGLLLCRVDSDPPAQL
RLLHGDRLVASTLQGVGGPEGSSPRLHVAVAPNTLRLEIHGAMLEDEGVYICEASNTLGQASASADFD
AQSCECAGVARGYRAGGAAGEPDLPCVDHSPGPAHLHMVPGWAAAPGCPLHPPAQRHSQGCHLLPLRC
GPPWSGTPPLQTYHLGRPHAPRNLRLTYLLESHGGQLALVLCTVDSRPPAQLALSHAGRLLASSTAAS
VPNTLRLELRGPQPRDEGFYSCSARSPLGQANTSLELRLEVRVILAPEAAVPEGAPITVTCADPAAHA
PTLYTWYHNGRWLQEGPAASLSFLVATRAHAGAYSCQAQDAQGTRSSRPAALQVLCAPQDAVLSSFRD
SRARSMAVIQCTVDSEPPAELALSHDGKVLATSSGVHSLASGTGHVQVARNALRLQVQDVPAGDDTYV
CTAQNLLGSISTIGRIJQVEGEWRWAEPGLDVPEGAALNLSCRLLGGPGPVGNSTFAWFWNDRRLHAE
PVPTLAFTHVARAQAGMYHCLAELPTGAAASAPVMLRVLYPPKTPTMMVFVEPEGGLRGILDCRVDSE
PLASLTLHLGSPLVASSQPQGAPAESHIHVLASPNALRVDIEALRPSDQGEYICSASNVLGPASTSTY
FGVRALHRLHQFQQLLWVLGLLVGLLLLLLGLGACYTWRRRRVCKQSMGENPGRASSFHLLPLAGSSR

| | | |
|---|---|---|
| NOV14k, | | |
| SNP13377617 of | SEQ ID NO: 417 | 5103 bp |
| CG51821-01, | ORF Start: ATG at 1 | ORF Stop: TGA at 5101 |
| DNA Sequence | SNP Pos: 4881 | SNP Change: C to T |

ATGGGCTTCTTGCCCAAGCTTCTCCTCCTGGCCTCAGCCGTTCTTCCCCCAGGCCAGGCCTCATGGGG
CGTCTCCAGTCCCCAGGACGTGCAGGGTGTGAAGGGGTCTTGCCTGCTTATCCCCTGCATCTTCAGCT
CAGGTGGTGAGCCACTCGGCGGACCCCAAGCTGGTGGAGGCCCGCTTCCGCGGCCGCACCGAGTTCAT
GGGGAACCCCGAGCACAGGGTGTGCAACCTGCTGCTGAAGGACCTGCAGCCCGAGGACTCTGGTTCCT
ACAACTTCCGCTTCGAGATCAGTGAGGTCAACCGCTGGTCAGATGTGAAAGGCACCTTGGTCACAGTA
ACAGGTGATCCCAGGGTGCCCACCATTGCCTCCCCGGTGGAGCTTCTCGAGGGCACAGAGGTGGACTT
CAACTGCTCCACTCCCTACGTATGCCTGCAGGAGCAGGTCAGACTGCAGTGGCAAGGCCAGGACCCTG
CTCGCTCTGTCACCTTCAACAGCCAGAAGTTTGAGCCCACCGGCGTCGGCCACCTGGAGACCCTCCAC
ATGGCCATGTCCTGGCAGGACCACGGCCGGATCCTGCGCTGCCAGCTCTCCGTGGCCAATCACAGGGC
TCAGAGCGAGATTCACCTCCAAGTGAAGTGTGCCCCCAAGGGTGTGAAGATCCTCCTCAGCCCCTCGG
GGAGGAACATCCTTCCAGGTGAGCTGGTCACACTCACCTGCCAGGTGAACAGCAGCTACCCTGCAGTC
AGTTCCATTAAGTGGCTCAAGGATGGGGTACGCCTCCAAACCAAGACTGGTGTGCTGCACCTGCCCCA
GGCAGCCTGGAGCGATGCTGGCGTCTACACCTGCCAAGCTGAGAACGGCGTGGGCTCTTTGGTCTCAC
CCCCCATCAGCCTCCACATCTTCGTGGCTGAGGTCCAGGTGAGCCCAGCAGGTCCCATCCTGGAGAAC
CAGACAGTGACACTAGTCTGCAACACACCCAATGAGGCACCCAGTGATCTCCGCTACAGCTGGTACAA
CTGGCTTCTACTTCTGTGAGGTGCAGAACGTCCATGGCAGCGAGCGCTCGGGCCCTGTCAGCGTGGTA
GTCACAGACCCGCCTCTCACTCCAGTCCTGACAGCCTTCCTGGAGACCCAGGCGGGACTTGTGGGCAT
CCTTCACTGCTCTGTGGTCAGTGAGCCCCTGGCCACACTGGTGCTGTCACATGGGGGTCATATCCTGG
CCTCCACCTCCGGGGACAGTGATCACAGCCCACGCTTCAGTGGTACCTCTGGTCCCAACTCCCTGCGC
CTGGAGATCCGAGACCTGGAGGAAACTGACAGTGGGGAGTACAAGTGCTCAGCCACCAACTCCCTTGG
AAATGCAACCTCCACCCTGGACTTCCATGCCAATGTCGCCCGTCTCCTCATCAGCCCGGCAGCCGAGG
TGGTGGAAGGACAGGCAGTGACACTGAGCTGCAGAAGCGGCCTAAGCCCCACACCTGATGCCCGCTTC
TCCTGGTACCTGAATGGAGCCCTGCTTCACGAGGGTCCCGGCAGCAGCCTCCTGCTCCCCGCGGCCTC
CAGCACTGACGCCGGCTCATACCACTGCCGGGCCCGGGACGGCCACAGTGCCAGTGGCCCCTCTTCGC
CAGCTGTTCTCACTGTGCTCTGTGAGCAGCCACCACGACAACCAACATTCACCACCAGGCTGGACCTT
GATGCCGCTGGGGCCGGGGCTGGACGGCGAGGCCTCCTTTTGTGCCGTGTGGACAGCGACCCCCCCGC
CAGGCTGCAGCTGCTCCACAAGGACCGTGTTGTGGCCACTTCCCTGCCATCAGGGGGTGGCTGCAGCA
CCTGTGGGGGCTGTTCCCCACGCATGAAGGTCACCAAAGCCCCCAACTTGCTGCGTGTGGAGATTCAC
AACCCTTTGCTGGAAGAGGAGGGCTTGTACCTCTGTGAGGCCAGCAATGCCCTGGGCAACGCCTCCAC
CTCAGCCACCTTCAATGGCCAGGCCACTGTCCTGGCCATTGCACCATCACACACACTTCAGGAGGGCA
CAGAAGCCAACTTGACTTGCAACGTGAGCCGGGAAGCTGCTGGCAGCCCTGCTAACTTCTCCTGGTTC
CGAAATGGGGTGCTGTGGGCCCAGGGTCCCCTGGAGACCGTGACACTGCTGCCCGTGGCCAGAACTGA
TGCTGCCCTTTACGCCTGCCGCATCCTGACTGAGGCTGGTGCCCAGCTCTCCACTCCCGTGCTCCTGA
GTGTACTCTATCCCCCGGACCGTCCAAAGCTGTCAGCCCTCCTAGACATGGGCCAGGGCCACATGGCT
CTGTTCATCTGCACTGTGGACAGCCGCCCCCTGGCCTTGCTGGCCTTGTTCCATGGGGAGCACCTCCT
GGCCACCAGCCTGGGTCCCCAGGTCCCATCCCATGGTCGGTTCCAGGCTAAAGCTGAGGCCAACTCCC
TGAAGTTAGAGGTCCGAGAACTGGGCCTTGGGGACTCTGGCAGCTACCGCTGTGAGGCCACAAATGTT
CTTGGATCATCCAACACCTCACTCTTCTTCCAGGTCCGAGGTGCCTGGGTCCAGGTGTCACCATCACC
TGAGCTCCAAGAGGGCCAGGCTGTGGTCCTGAGCTGCCAGGTACACACAGGAGTCCCAGAGGGGACCT
CATATCGTTGGTATCGGGATGGCCAGCCCCTCCAGGAGTCGACCTCGGCCACGCTCCGCTTTGCAGCC
ATAACTTTGACACAAGCTGGGGCCTATCATTGCCAAGCCCAGGCCCCAGGCTCAGCCACCACGAGCCT
AGCTGCACCCATCAGCCTCCACGTGTCCTGTAAGGATGCCCCACGCCACGTCACACTCACTACCCTGA
TGGACACAGGCCCTGGACGACTGGGCCTCCTCCTGTGCCGTGTGGACAGTGACCCTCCGGCCCAGCTG
CGGCTGCTCCACGGGGATCGCCTTGTGGCCTCCACCCTACAAGGTGTGGGGGGACCCGAAGGCAGCTC
TCCCAGGCTGCATGTGGCTGTGGCCCCCAACACACTGCGTCTGGAGATCCACGGGGCTATGCTGGAGG
ATGAGGGTGTCTATATCTGTGAGGCCTCCAACACCCTGGGCCAGGCCTCGGCCTCAGCTGACTTCGAC
GCTCAAAGCTGTGAATGTGCAGGTGTGGCCCGGGGCTACCGTGCGGGAGGGGCAGCTGGTGAACCTGA
CCTGCCTTGTGTGGACCACTCACCCGGCCCAGCTCACCTACACATGGTACCAGGATGGGCAGCAGCGC
GGGCCCCCCTGGTCGGGCACCCCGCCTCTCCAGACCTATCACCTTGGACGTCCTCACGCGCCCCGCAA
CCTGCGCCTGACCTACCTCCTGGAGAGCCATGGCGGGCAGCTGGCCCTGGTACTGTGCACTGTGGACA
CCGCCCGCACCGCCCAGCTGGCCCTCAGCCACGCCGGTCGCCTCTTGGCCTCCTCGACAGCAGCCTCT
GTCCCCAACACCCTGCGCCTGGAGCTGCGAGGGCCACAGCCCAGGGATGAGGGTTTCTACAGCTGCTC
TGCCCGCAGCCCTCTGGGCCAGGCCAACACGTCCCTGGAGCTGCGGCTGGAGGTGCGGGTGATCCTGG
CCCACACTCTATACTTGGTACCACAACGGTCGTTGGCTGCAGGAGGGTCCAGCTGCCTCACTCTCATT
CCTGGTGGCCACGCGGGCTCATGCAGGCGCCTACTCTTGCCAGGCCCAGGATGCCCAGGGCACCCGCA
GCTCCCGTCCTGCTGCCCTGCAAGTCCTCTGTGCCCCTCAGGACGCTGTCCTGTCCTCCTTCCGGGAC
TCCAGGGCCAGATCCATGGCTGTGATACAGTGCACTGTGGACAGTGAGCCACCTGCTGAGCTGGCCCT
ATCTCATGATGGCAAGGTGCTGGCCACGAGCAGCGGGGTCCACAGCTTGGCATCAGGGACAGGCCATG
TCCAGGTGGCCCGAAACGCCCTACGGCTGCAGGTGCAAGATGTGCCTGCAGGTGATGACACCTATGTT
TGCACAGCCCAAAACTTGCTGGGCTCAATCAGCACCATCGGGCGGTTGCAGGTAGAAGGTGAGTGGCG
CGTGGTGGCAGAGCCTGGCCTGGACGTGCCTGAGGGCGCTGCCCTGAACCTCAGCTGCCGCCTCCTGG
GTGGCCCTGGGCCTGTGGGCAACTCCACCTTTGCATGGTTCTGGAATGACCGGCGGCTGCACGCGGAG
CCTGTGCCCACTCTCGCCTTCACCCACGTGGCTCGTGCTCAAGCTGGGATGTACCACTGCCTGGCTGA
GCTCCCCACTGGGGCTGCTGCCTCTGCTCCAGTCATGCTCCGTGTGCTCTACCCTCCCAAGACGCCCA
CCGCTCGCCAGCCTGACTCTCCACCTTGGCAGTCGACTGGTGGCCTCCAGTCAGCCCCAGGGTGCTCC
TGCAGAGCCACACATCCATGTCCTGGCTTCCCCCAATGCCCTGAGGGTGGACATCGAGGCGCTGAGGC
CCAGCGACCAAGGGGAATACATCTGTTCTGCCTCAAATGTCCTGGGCCCTGCTTCTACCTCCACCTAC
TTTGGGGTCAGAGCCCTGCACCGCCTGCATCAGTTCCAGCAGCTGCTCTGGGTCCTGGGACTGCTGGT
GGGCCTCCTGCTCCTGCTGTTGGGCCTGGGGGCCTGCTACACCTGGAGAAGGAGGCGTGTTTGTAAGC
AGAGCATGGGCGAGAATCCAGGGCGGGCATCGTCTTTCCATTTACTGCCTCTAGCTGGGTCTTCAAGG
TGA

| | | | |
|---|---|---|---|
| NOV14k, | | | |
| SNP1 3377617 of | | | |
| CG51821-01, | SEQ ID NO: 4181 | | MW at 180833.0kD |
| Protein Sequence | SNP Pos: 1627 | 1700 aa | SNP Change: Ala to Ala |

MGFLPKLLLLASAVLPPGQASWGVSSPQDVQGVKGSCLLIPCIFSFPADVEVPDGITAIWYYDYSGQR
QVVSHSADPKLVEARFRGRTEFMGNPEHRVCNLLLKDLQPEDSGSYNFRFEISEVNRWSDVKGTLVTV
TGDPRVPTIASPVELLEGTEVDFNCSTPYVCLQEQVRLQWQGQDPARSVTFNSQKFEPTGVGHLETLH
MAMSWQDHGRILRCQLSVANHRAQSEIHLQVKCAPKGVKILLSPSGRNILPGELVTLTCQVNSSYPAV
SSIKWLKDGVRLQTKTGVLHLPQAAWSDAGVYTCQAENGVGSLVSPPISLHIFVAEVQVSPAGPILEN
QTVTLVCNTPNEAPSDLRYSWYKNHVLLEDAHSHTLRLHLATRADTGFYFCEVQNVHGSERSGPVSVV
VTDPPLTPVLTAFLETQAGLVGILHCSVVSEPLATLVLSHGGHILASTSGDSDHSPRFSGTSGPNSLR
LEIRDLEETDSGEYKCSATNSLGNATSTLDFHANVARLLISPAAEVVEGQAVTLSCRSGLSPTPDARF
SWYLNGALLHEGPGSSLLLPAASSTDAGSYHCPARDGHSASGPSSPAVLTVLCEQPPRQPTFTTRLDL
DAAGAGAGRRGLLLCRVDSDPPARLQLLHKDRVVATSLPSGGGCSTCGGCSPRMKVTKAPNLLRVEIH
NPLLEEEGLYLCEASNALGNASTSATFNGQATVLAIAPSHTLQEGTEANLTCNVSREAAGSPANFSWF
RNGVLWAQGPLETVTLLPVARTDAALYACRILTEAGAQLSTPVLLSVLYPPDRPKLSALLDMGQGHMA
LFICTVDSRPLALLALFHGEHLLATSLGPQVPSHGRFQAKAEANSLKLEVRELGLGDSGSYRCEATNV
LGSSNTSLFFQVRGAWVQVSPSPELQEGQAVVLSCQVHTGVPEGTSYRWYRDGQPLQESTSATLRFAA
ITLTQAGAYHCQAQAPGSATTSLAAPISLHVSCKDAPRHVTLTTLMDTGPGRLGLLLCRVDSDPPAQL
RLLHGDRLVASTLQGVGGPEGSSPRLHVAVAPNTLRLEIHGANLEDEGVYICEASNTLGQASASADFD
AQSCECAGVARGYRAGGAAGEPDLPCVDHSPGPAHLHMVPGWAAAPGCPLHPPAQRHSQGCHLLPLRC
GPPWSGTPPLQTYHLGRPHAPRNLRLTYLLESHGGQLALVLCTVDSRPPAQLALSHAGRLLASSTAAS
VPNTLRLELRGPQPRDEGFYSCSARSPLGQANTSLELRLEVRVILAPEAAVPEGAPITVTCADPAAHA
PTLYTWYHNGRWLQEGPAASLSFLVATRAHAGAYSCQAQDAQGTRSSRPAALQVLCAPQDAVLSSFRD
SRARSMAVIQCTVDSEPPAELALSHDGKVLATSSGVHSLASGTGHVQVAPNALRLQVQDVPAGDDTYV
CTAQNLLGSISTIGRLQVEGEWRVVAEPGLDVPEAGAALNLSCRLLGPGPVGNSTFAWFWNDRRLHAE
PVPTLAFTHVARAQAGMYHCLAELPTGAAASAPVMLRVLYPPKTPTMMVFVEPEGGLRGILDCRVDSE
PLASLTLHLGSRLVASSQPQGAPAEPHIHVLASPNALRVDIEALRPSDQGEYICSASNVLGPASTSTY
FGVRALHRLHQFQQLLWVLGLLVGLLLLLLGLGACYTWRRRRVCKQSMGENPGRASSFHLLPTAGSSR

A ClustalW comparison of the above protein sequences yields the following sequence alignment shown in Table 14B.

TABLE 14B

Comparison of the NOV14 protein sequences.

| | |
|---|---|
| NOV14a | MGFLPKLLLLASAVLPPGQASWGVSSPQDVQGVKGSCLLIPCIFSFPADVEVPDGITAIW |
| NOV14b | ------------------RSSWGVSSPQDVQGVKGSCLLIPCIFSFPADVEVPDGITAIW |
| NOV14c | ------------------RSSWGVSSPQDVQGVKGSCLLIPCIFSFPADVEVPDGITAIW |
| NOV14d | ------------------RSSWGVSSPQDVQGVKGSCLLIPCIFSFPADVEVPDGITAIW |
| NOV14e | --------------------SWGVSSPQDVQGVKGSCLLIPCIFSFPADVEVPDGITAIW |
| NOV14f | --------------------SWGVSSPQDVQGVKGSCLLIPCIFSFPADVEVPDGITAIW |
| NOV14g | MGFLPKLLLLAS-FFPAGQASWGVSSPQDVQGVKGSCLLIPCIFSFPADVEVPDGITAIW |
| NOV14a | YYDYSGQRQVVSHSADPKLVEARFRGRTEFMGNPEHRVCNLLLKDLQPEDSGSYNFRFEI |
| NOV14b | YYDYSGQRQVVSHSADPKLVEARFRGRTEFMGNPEHRVCNLLLKDLQPEDSGSYNFRFEI |
| NOV14c | YYDYSGQRQVVSHSADPKLVEARFRGRTEFMGNPEHRVCNLLLKDLQPEDSGSYNFRFEI |
| NOV14d | YYDYSGQRQVVSHSVDPKLVEARFRGRTEFMGNPEHRVCNLLLKDLQPEDSGSYNFRFEI |
| NOV14e | YYDYSGQRQVVSHSADPKLVEARFRGRTEFMGNPEHRVCNLLLKDLQPEDSGSYNFRFEI |
| NOV14f | YYDYSGQRQVVSHSVDPKLVEARFRGRTEFMGNPEHRVCNLLLKDLQPEDSGSYNFRFEI |
| NOV14g | YYDYSGQRQVVSHSADPKLVEARFRGRTEFMGNPEHRVCNLLLKDLQPEDSGSYNFRFEI |
| NOV14a | SEVNRWSDVKGTLVTVTGDPRVPTIASPVELLEGTEVDFNCSTPYVCLQEQVRLQWQGQD |
| NOV14b | SEVNRWSDVKGTLVTVTEEPRVPTIASPVELLEGTEVDFNCSTPYVCLQEQVRLQWQGQD |
| NOV14c | SEVNRWSDVKGTLVTVTEEPRVPTIASPVELLEGTEVDFNCSTPYVCLQEQVRLQWQGQD |
| NOV14d | SEVNRWSDVKGTLVTVTEEPRVPTIASPVELLEGTEVDFNCSTPYVCLQEQVRLQWQGQD |
| NOV14e | SEVNRWSDVKGTLVTVTEEPRVPTIASPVELLEGTEVDFNCSTPYVCLQEQVRLQWQGQD |
| NOV14f | SEVNRWSDVKGTLVTVTEEPRVPTIASPVELLEGTEVDFNCSTPYVCLQEQVRLQWQGQD |
| NOV14g | SEVNRWSDVKGTLVTVTEEPRVPTIASPVELLEGTEVDFNCSTPYVCLQEQVRLQWQGQD |
| NOV14a | PARSVTFNSQKFEPTGVGHLETLHMAMSWQDHGRILRCQLSVANHRAQSEIHLQVKCAPK |
| NOV14b | PARSVTFNSQKFEPTGVGHLETLHMAMSWQDHGRILRCQLSVANHRAQSEIHLQVKYAPR |
| NOV14c | PARSVTFNSQKFEPTGVGHLETLHMAMSWQDHGRILRCQLSMANHRAQSEIHLQVKYAPR |
| NOV14d | PARSVTFNSQKFEPTGVGHLETLHMAMSWQDHGRILRCQLSMANHRAQSEIHLQVKYAPK |
| NOV14e | PARSVTFNSQKFEPTGVGHLETLHMAMSWQDHGRILRCQLSVANHRAQSEIHLQVKYAPR |
| NOV14f | PARSVTFNSQKFEPTGVGHLETLHNAMSWQDHGRILRCQLSMANHRAQSEIHLQVKYAPK |
| NOV14g | PARSVTFNSQKFEPTGVGHLETLHMAMSWQDHGRILRCQLSVANHRAQSEIHLQVKYAPK |
| NOV14a | GVKILLSPSGRNILPGELVTLTCQVNSSYPAVSSIKWLKDGVRLQTKTGVLHLPQAAWSD |
| NOV14b | GVKILLSPSGRNILPVD------------------------------------------- |
| NOV14c | GVKILLSPSGRNILPVD------------------------------------------- |
| NOV14d | GVKILLSPSGRNILPVD------------------------------------------- |
| NOV14e | GVKILLSPSGRNILP--------------------------------------------- |
| NOV14f | GVKILLSPSGRNILP--------------------------------------------- |
| NOV14g | GVKILLSPSGRNILPGELVTLTCQVNSSYPAVSSIKWLKDGVRLQTKTGVLHLPQAAWSD |
| NOV14a | AGVYTCQAENGVGSLVSPPISLHIFVAEVQVSPAGPILENQTVTLVCNTPNEAPSDLRYS |
| NOV14b | ------------------------------------------------------------ |
| NOV14c | ------------------------------------------------------------ |
| NOV14d | ------------------------------------------------------------ |
| NOV14e | ------------------------------------------------------------ |
| NOV14f | ------------------------------------------------------------ |
| NOV14g | AGVYTCQAENGVGSLVSPPISLHIFMAEVQVSPAGPILENQTVTLVCNTPNEAPSDLRYS |
| NOV14a | WYKNHVLLEDAHSHTLRLHLATRADTGFYFCEVQNVHGSERSGPVSVVVTDPPLTPVLTA |
| NOV14b | ------------------------------------------------------------ |
| NOV14c | ------------------------------------------------------------ |
| NOV14d | ------------------------------------------------------------ |
| NOV14e | ------------------------------------------------------------ |
| NOV14f | ------------------------------------------------------------ |
| NOV14g | WYKNHVLLEDAHSHTLRLHLATRADTGFYFCEVQNVHGSERSGPVSVVVNHPPLTPVLTA |
| NOV14a | FLETQAGLVGILHCSVVSEPLATLVLSHGGHILASTSGDSDHSPRFSGTSGPNSLRLEIR |
| NOV14b | ------------------------------------------------------------ |
| NOV14c | ------------------------------------------------------------ |
| NOV14d | ------------------------------------------------------------ |
| NOV14e | ------------------------------------------------------------ |
| NOV14f | ------------------------------------------------------------ |
| NOV14g | FLETQAGLVGILHCSVVSEPLATLVLSHGGHILASTSGDSDHSPRFSGTSGPNSLRLEIR |
| NOV14a | DLEETDSGEYKCSATNSLGNATSTLDFHANAARLLISPAAEVVEGQAVTLSCRSGLSPTP |
| NOV14b | ------------------------------------------------------------ |
| NOV14c | ------------------------------------------------------------ |
| NOV14d | ------------------------------------------------------------ |
| NOV14e | ------------------------------------------------------------ |
| NOV14f | ------------------------------------------------------------ |
| NOV14g | DLEETDSGEYKCSATNSLGNATSTLDFHANAARLLISPAAEVVEGQAVTLSCRSGLSPTP |
| NOV14a | DARFSWYLNGALLHEGPGSSLLLPAASSTDAGSYHCRARDGHSASGPSSPAVLTVLCEQP |
| NOV14b | ------------------------------------------------------------ |
| NOV14c | ------------------------------------------------------------ |
| NOV14d | ------------------------------------------------------------ |
| NOV14e | ------------------------------------------------------------ |
| NOV14f | ------------------------------------------------------------ |
| NOV14g | DARFSWYLNGALLHEGPGSSLLLPAASSTDAGSYHCRARDGHSASGPSSPAVLTVLY--P |
| NOV14a | PRQPTFTTRLDLDAAGAGAGRRGLLLCRVDSDPPARLQLLHKDRVVATSLPSGGGCSTCG |
| NOV14b | ------------------------------------------------------------ |
| NOV14c | ------------------------------------------------------------ |
| NOV14d | ------------------------------------------------------------ |
| NOV14e | ------------------------------------------------------------ |
| NOV14f | ------------------------------------------------------------ |
| NOV14g | PRQPTFTTRLDLDAAGAGAGRRGLLLCRVDSDPPARLQLLHKDRVVATSLPSGGGCSTCG |
| NOV14a | GCSPRMKVTKAPNLLRVEIHNPLLEEEGLYLCEASNALGNASTSATFNGQATVLAIAPSH |
| NOV14b | ------------------------------------------------------------ |
| NOV14c | ------------------------------------------------------------ |
| NOV14d | ------------------------------------------------------------ |
| NOV14e | ------------------------------------------------------------ |
| NOV14f | ------------------------------------------------------------ |
| NOV14g | GCSPRMKVTKAPNLLRVEIHNPLLEEEGLYLCEASNALGNASTSATFNGQATVLAIAPSH |
| NOV14a | TLQEGTEANLTCNVSREAAGSPANFSWFRNGVLWAQGPLETVTLLPVARTDAALYACRIL |
| NOV14b | ------------------------------------------------------------ |
| NOV14c | ------------------------------------------------------------ |
| NOV14d | ------------------------------------------------------------ |
| NOV14e | ------------------------------------------------------------ |
| NOV14f | ------------------------------------------------------------ |
| NOV14g | TLQEGTEANLTCNVSREAAGSPANFSWFRNGVLWAQGPLETVTLLPVARTDAALYACRIL |
| NOV14a | TEAGAQLSTPVLLSVLYPPDRPKLSALLDMGQGHMALFICTVDSRPLALLALFHGEHLLA |
| NOV14b | ------------------------------------------------------------ |
| NOV14c | ------------------------------------------------------------ |
| NOV14d | ------------------------------------------------------------ |
| NOV14e | ------------------------------------------------------------ |
| NOV14f | ------------------------------------------------------------ |
| NOV14g | TEAGAQLSTPVLLSVLYPPDRPKLSALLDMGQGHMALFICTVDSRPLALLALFHGEHLLA |
| NOV14a | TSLGPQVPSHGRFQAKAEANSLKLEVRELGLGDSGSYRCEATNVLGSSNTSLFFQVRGAW |
| NOV14b | ------------------------------------------------------------ |
| NOV14c | ------------------------------------------------------------ |
| NOV14d | ------------------------------------------------------------ |
| NOV14e | ------------------------------------------------------------ |
| NOV14f | ------------------------------------------------------------ |
| NOV14g | TSLGPQVPSHGRFQAKAEANSLKLEVRELGLGDSGSYRCEATNVLGSSNTSLFFQVRGAW |
| NOV14a | VQVSPSPELQEGQAVVLSCQVHTGVPEGTSYRWYRDGQPLQESTSATLRFAAITLTQAGA |
| NOV14b | ------------------------------------------------------------ |
| NOV14c | ------------------------------------------------------------ |
| NOV14d | ------------------------------------------------------------ |
| NOV14e | ------------------------------------------------------------ |
| NOV14f | ------------------------------------------------------------ |
| NOV14g | VQVSPSPELQEGQAVVLSCQVHTGVPEGTSYRWYRDGQPLQESTSATLRFAAITLTQAGA |
| NOV14a | YHCQAQAPGSATTSLAAPISLBVSCKDAPRHVTLTTLMDTGPGRLGLLLCRVDSDPPAQL |
| NOV14b | ------------------------------------------------------------ |
| NOV14c | ------------------------------------------------------------ |
| NOV14d | ------------------------------------------------------------ |
| NOV14e | ------------------------------------------------------------ |
| NOV14f | ------------------------------------------------------------ |
| NOV14g | YHCQAQAPGSATTSLAAPISLHVSY--APRHVTLTTLMDTGPGRLGLLLCRVDSDPPAQL |
| NOV14a | RLLHGDRLVASTLQGVGGPEGSSPRLHVAVAPNTLRLEIHGAMLEDEGVYICEASNTLGQ |
| NOV14b | ------------------------------------------------------------ |
| NOV14c | ------------------------------------------------------------ |
| NOV14d | ------------------------------------------------------------ |
| NOV14e | ------------------------------------------------------------ |
| NOV14f | ------------------------------------------------------------ |
| NOV14g | RLLHGDRLVASTLQGVGGPEGSSPRLHVAVAPNTLRLETHGAMLEDEGVYICEASNTLGQ |
| NOV14a | ASASADFDAQSCECAGVARGYRAGGAAGEPDLPCVDHSPGPAHLHMVPGWAAAPGCPLHP |
| NOV14b | ------------------------------------------------------------ |
| NOV14c | ------------------------------------------------------------ |
| NOV14d | ------------------------------------------------------------ |
| NOV14e | ------------------------------------------------------------ |
| NOV14f | ------------------------------------------------------------ |
| NOV14g | ASASADFDAQAVNVQVWPGATVREGQLVN--LTCLVWTTHPAQLTYTWYQDGQQRLDAHS |
| NOV14a | PAQRHSQGCHLLPLRCG--PPWSGTPPLQTYHLGRPHAPRNLRLTYLLESHGGQLALVLC |
| NOV14b | ------------------------------------------------------------ |
| NOV14c | ------------------------------------------------------------ |
| NOV14d | ------------------------------------------------------------ |
| NOV14e | ------------------------------------------------------------ |
| NOV14f | ------------------------------------------------------------ |
| NOV14g | IPLPNVTVRDATSYRCGVGPPGRAPRLSRPITLDVLYAPRNLRLTYLLESHGGQLALVLC |
| NOV14a | TVDSRPPAQLALSHAGRLLASSTAASVPNTLRLELRGPQPRDEGFYSCSARSPLGQANTS |
| NOV14b | ------------------------------------------------------------ |
| NOV14c | ------------------------------------------------------------ |
| NOV14d | ------------------------------------------------------------ |
| NOV14e | ------------------------------------------------------------ |
| NOV14f | ------------------------------------------------------------ |
| NOV14g | TVDSRPPAQLALSHAGRLLASSTAASVPNTLRLELRGPQPRDEGFYSCSARSPLGQANTS |
| NOV14a | LELRLE-VRVILAPEAAVPEGAPITVTCADPAAHAPTLYTWYHNGRWLQEGPAASLSFLV |
| NOV14b | ------------------------------------------------------------ |
| NOV14c | ------------------------------------------------------------ |
| NOV14d | ------------------------------------------------------------ |
| NOV14e | ------------------------------------------------------------ |
| NOV14f | ------------------------------------------------------------ |
| NOV14g | LELRLEGVRVILAPEAAVPEGAPITVTCADPAAHAPTLYTWYHNGRWLQEGPAASLSFLV |
| NOV14a | ATRAHAGAYSCQAQDAQGTRSSRPAALQVLCAPQDAVLSSFRDSRARSMAVIQCTVDSEP |
| NOV14b | ------------------------------------------------------------ |
| NOV14c | ------------------------------------------------------------ |
| NOV14d | ------------------------------------------------------------ |
| NOV14e | ------------------------------------------------------------ |
| NOV14f | ------------------------------------------------------------ |
| NOV14g | ATPAHAGAYSCQAQDAQGTRSSRPAALQVLYAPQDAVLSSFRDSRARSMAVIQCTVDSEP |
| NOV14a | PAELALSHDGKVLATSSGVHSLASGTGHVQVARNALRLQVQDVPAGDDTYVCTAQNLLGS |
| NOV14b | ------------------------------------------------------------ |
| NOV14c | ------------------------------------------------------------ |
| NOV14d | ------------------------------------------------------------ |
| NOV14e | ------------------------------------------------------------ |
| NOV14f | ------------------------------------------------------------ |
| NOV14g | PAELALSHDGKVLATSSGVHSLASGTGHVQVARNALRLQVQDVPAGDDTYVCTAQNLLGS |
| NOV14a | ISTIGRLQVEGEWRVVAEPGLDVPEGAALNLSCRLLGGPGPVGNSTFAWFWNDRRLHAEP |
| NOV14b | ------------------------------------------------------------ |
| NOV14c | ------------------------------------------------------------ |
| NOV14d | ------------------------------------------------------------ |
| NOV14e | ------------------------------------------------------------ |
| NOV14f | ------------------------------------------------------------ |
| NOV14g | ISTIGRLQVEGAR-VVAEPGLDVPEGAALNLSCRLLGGPGPVGNSTFAWFWNDRRLHAEP |
| NOV14a | VPTLAFTHVARAQAGMYHCLAELPTGAAASAPVMLRVLYPPKTPTMMVFVEPEGGLRGIL |
| NOV14b | ------------------------------------------------------------ |
| NOV14c | ------------------------------------------------------------ |
| NOV14d | ------------------------------------------------------------ |
| NOV14e | ------------------------------------------------------------ |
| NOV14f | ------------------------------------------------------------ |
| NOV14g | VPTLAFTHVARAQAGMYHCLAELPTGAAASAPVMLRVLYPPKTPTNMVFVEPEGGLRGIL |
| NOV14a | DCRVDSEPLASLTLHLGSRLVASSQPQGAPAEPHIHVLASPNALRVDIEALRPSDQGEYI |
| NOV14b | ------------------------------------------------------------ |
| NOV14c | ------------------------------------------------------------ |
| NOV14d | ------------------------------------------------------------ |
| NOV14e | ------------------------------------------------------------ |
| NOV14f | ------------------------------------------------------------ |
| NOV14g | DCRVDSEPLASLTLHLGSRLVASSQPQGAPAEPHIHVLASPNALRVDIEALRPSDQGEYI |
| NOV14a | CSASNVLGPASTSTYFGVPALHRLHQFQQLLWVLGLLVGLLLLLLGLGACYTWRRRRVCK |
| NOV14b | ------------------------------------------------------------ |
| NOV14c | ------------------------------------------------------------ |
| NOV14d | ------------------------------------------------------------ |
| NOV14e | ------------------------------------------------------------ |
| NOV14f | ------------------------------------------------------------ |
| NOV14g | CSASNVLGSASTSTYFGVPALHRLHQFQQLLWVLGLLVGLLLLLLGLGACYTWRRRRVCK |
| NOV14a | QSMGENP-----GEASSFHLLPLAGSSR--------- |
| NOV14b | ------------------------------------- |
| NOV14c | ------------------------------------- |
| NOV14d | ------------------------------------- |
| NOV14e | ------------------------------------- |
| NOV14f | ------------------------------------- |
| NOV14g | QSMGENSVEMAFQKETTQLIDPDAATCETSTCAPPLG |
| NOV14a | (SEQ ID NO: 398) |
| NOV14b | (SEQ ID NO: 400) |
| NOV14c | (SEQ ID NO: 402) |
| NOV14d | (SEQ ID NO: 404) |
| NOV14e | (SEQ ID NO: 406) |
| NOV14f | (SEQ ID NO: 408) |
| NOV14g | (SEQ ID NO: 410) |

Further analysis of the NOV14a protein yielded the following properties shown in Table 14C.

TABLE 14C
Protein Sequence Properties NOV14a

| | |
|---|---|
| SignalP analysis: | Cleavage site between residues 21 and 22 |
| PSORT II analysis: | |

| PSG: | a new signal peptide prediction method | |
|---|---|---|
| | N-region: | length 6; pos. chg 1; neg. chg 0 |
| | H-region: | length 22; peak value 9.20 |
| | PSG score: | 4.80 |

| | |
|---|---|
| GvH: | von Heijne's method for signal seq. recognition |
| | GvH score (threshold: −2.1): 2.74 |
| | possible cleavage site: between 20 and 21 |

>>> Seems to have a cleavable signal peptide (1 to 20)

| ALOM: | Klein et al's method for TM region allocation | | |
|---|---|---|---|
| | Init position for calculation: 21 | | |
| | Tentative number of TMS(s) for the threshold 0.5: 3 | | |
| | Number of TMS(s) for threshold 0.5: 1 | | |
| | INTEGRAL | Likelihood = | Transmembrane |
| | | −11.25 | 1647-1663 |

| | |
|---|---|
| PERIPHERAL | Likelihood =0.69 (at 36) |

| | |
|---|---|
| | ALOM score: −11.25 (number of TMSs: 1) |
| MTOP: | Prediction of membrane topology (Hartmann et al.) |
| | Center position for calculation: 10 |
| | Charge difference: −2.0 C(0.0)-N(2.0) |
| | N >= C: N-terminal side will be inside |

>>> membrane topology: type 1a (cytoplasmic tail 1664 to 1700)

| MITDISC: | discrimination of mitochondrial targeting seq | | | |
|---|---|---|---|---|
| | R content: | 0 | Hyd Moment(75): | 6.81 |
| | Hyd Moment(95): | 7.54 | G content: | 3 |
| | D/E content: | 1 | S/T content: | 4 |

| | |
|---|---|
| | Score: −4.95 |
| Gavel: | prediction of cleavage sites for mitochondrial preseq |
| | cleavage site motif not found |

NUCDISC: discrimination of nuclear localization signals

| | |
|---|---|
| | pat4: RRRR (5) at 1671 |
| | pat7: none |
| | bipartite: none |
| | content of basic residues: 7.3% |
| | NLS Score: −0.16 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: 1667
Dileucine motif in the tail: found

LL at 1692

checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 55.5 |
| COIL: | Lupas's algorithm to detect coiled-coil regions |
| | total: 0 residues |

Final Results (k = 9/23):

33.3%: endoplasmic reticulum
22.2%: Golgi
22.2%: extracellular, including cell wall
11.1%: plasma membrane
11.1%: vesicles of secretory system

>> prediction for CG51821-01 is end (k = 9)

A search of the NOV14a protein against the Geneseq database, a proprietary database that contains sequences published in. patents and patent publication, yielded several homologous proteins shown in Table 14D.

TABLE 14D
Geneseq Results for NOV14a

| | | NOV14a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB05044 | Human NOV6 protein SEQ ID NO: 24 - | 1 . . . 1700 | 1700/1700 (100%) | 0.0 |
| | Homo sapiens, 1700 aa. | 1 . . . 1700 | 1700/1700 (100%) | |
| | [WO200190155-A2, 29-NOV-2001] | | | |
| AAE09448 | Human sbg24878Sla protein #1 - | 1 . . . 1683 | 1588/1695 (93%) | 0.0 |
| | Homo sapiens, 1709 aa. | 1 . . . 1672 | 1598/1695 (93%) | |
| | [WO200160850-A1, 23-AUG-2001] | | | |
| ME09449 | Human sbg24878Sla protein #2 - | 1 . . . 1671 | 1576/1683 (93%) | 0.0 |
| | Homo sapiens, 1694 aa. | 1 . . . 1660 | 1586/1683 (93%) | |
| | [WO200160850-A1, 23-AUG-2001] | | | |
| ABG10466 | Novel human diagnostic protein | 18 . . . 1671 | 1562/1666 (93%) | 0.0 |
| | #10457 - Homo sapiens, 1839 aa. | 162 . . . 1805 | 1573/1666 (93%) | |
| | [WO200175067-A2, 11-OCT-2001] | | | |
| ABP69461 | Human polypeptide SEQ ID NO | 482 . . . 1683 | 1118/1214 (92%) | 0.0 |
| | 1508 - Homo sapiens, 1263 aa. | 35 . . . 1232 | 1126/1214 (92%) | |
| | [WO200270539-A2, 12-SEP-2002] | | | |

In a BLAST search of public sequence databases, the NOV14a protein was found to have homology to the proteins shown in the BLASTP data in Table 14E.

TABLE 14E
Public BLASTP Results for NOV14a

| | | NOV14a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| Q9BZZ2 | Sialoadhesin precursor (Sialic acid | 1 . . . 1683 | 1594/1695 | (94%) | 0.0 |
| | binding Ig-like lectin-1) (Siglec- 1) | 1 . . . 1678 | 1603/1695 | (94%) | |
| | (CD169 antigen) - Homo sapiens | | | | |
| | (Human), 1709 aa. | | | | |
| Q62230 | Sialoadhesin precursor (Sialic acid | 8 . . . 1683 | 1167/1688 | (69%) | 0.0 |
| | binding Ig-like lectin-1) (Siglec- 1) | 7 . . . 1675 | 1333/1688 | (78%) | |
| | (Sheep erythrocyte receptor) (SER) - | | | | |
| | Mus musculus (Mouse), 1694 aa. | | | | |
| BAC56927 | FLJ00411 protein - Homo sapiens | 798 . . . 1091 | 288/294 | (97%) | e−162 |
| | (Human), 437 aa (fragment). | 17 . . . 308 | 289/294 | (97%) | |
| P35329 | B-cell receptor CD22 precursor (Leu- | 22 . . . 634 | 167/649 | (25%) | 5e−37 |
| | 14) (B-lymphocyte cell adhesion | 26 . . . 638 | 274/649 | (41%) | |
| | molecule) (BL-CAM) (Siglec-2) - Mus | | | | |
| | musculus (Mouse), 862 aa. | | | | |
| A46512 | CD22 homolog/B lymphocyte- | 22 . . . 634 | 163/653 | (24%) | 6e−35 |
| | restricted adhesion molecule - mouse, | 26 . . . 644 | 271/653 | (40%) | |
| | 868 aa. | | | | |

PFam analysis predicts that the NOV14a protein contains the domains shown in the Table 14F.

TABLE 14F
Domain Analysis of NOV14a

| | | Identities/ | |
|---|---|---|---|
| | | Similarities | |
| Pfam | NOV14a | for the | Expect |
| Domain | Match Region | Matched Region | Value |
| ig | 256 . . . 308 | 20/54 (37%) | 1.5e−11 |
| | | 43/54 (80%) | |
| ig | 340 . . . 393 | 17/55 (31%) | 4.3e−10 |
| | | 44/55 (80%) | |
| ig | 427 . . . 494 | 17/71 (24%) | 0.0082 |
| | | 51/71 (72%) | |
| ig | 525 . . . 578 | 16/55 (29%) | 2.1e−06 |
| | | 39/55 (71%) | |
| ig | 620 . . . 694 | 12/78 (15%) | 0.92 |
| | | 53/78 (68%) | |
| ig | 725 . . . 779 | 15/56 (27%) | 0.027 |
| | | 35/56 (62%) | |
| ig | 813 . . . 881 | 13/72 (18%) | 0.022 |
| | | 47/72 (65%) | |
| ig | 912 . . . 965 | 16/55 (29%) | 2.1e−07 |
| | | 39/55 (71%) | |
| ig | 1003 . . . 1074 | 14/75 (19%) | 0.023 |
| | | 52/75 (69%) | |
| ig | 1191 . . . 1248 | 13/61 (21%) | 0.0048 |
| | | 40/61 (66%) | |
| ig | 1278 . . . 1330 | 15/55 (27%) | 0.16 |
| | | 34/55 (62%) | |
| ig | 1364 . . . 1431 | 13/72 (18%) | 0.00084 |
| | | 50/72 (69%) | |

### Example 15

The NOV15 clone was analyzed, and the nucleotide and encoded polypeptide sequences are shown in Table 15A.

TABLE 15A
NOV15 Sequence Analysis

| | | |
|---|---|---|
| NOV15a, CG51992-05 | SEQ ID NO: 419 | 1511 bp |
| DNA Sequence | ORF Start: ATG at 339 | ORF Stop: TGA at 1203 |

AGAGAAGGAGCTAAGAAGCCTGTGGCTACTGAGAAGAGGCCAGCTAAGTGATGCTGGCATGTAGGAAG
CAGGCACACTACTTGTGGATGGGGAGCATGGTGACTGGAGAGACCAGTGAGGGCAATTTGCATCCTTT
GAGGGCAATTTGGTCTCCAAAGAACAGAGTGATCTGGAGGGAGTGACAGCCAAGAGTCAGCCAGAGCA
TCTCAGCCATCCCAGGAATCAGAGAAATCCAGGGAAGCCAGTCTTAATATGATGGAAACATCTCTGAA
CTTCTAAAAGACCAAGGTTGGCGTTTTAGCTCTATTAATTTTACTTCGTCTTGGCCAGAATTCACAAT
GACAACAGTGACAGTGACCACAGAAATTCCCCCAAGGGATAAGATGGAAGATAATTCTGCCTTGTATG
AGTCTACGTCCGCTCACATTATTGAAGAAACCGAGTATGTGAAAAAGATTCGAACTACTCTGCAAAAG
ATCAGGACCCAGATGTTTAAAGATGAAATAAGACATGACAGTACAAATCACAAACTAGATGCAAAGCT
GCAAGCCTCCAGAGAAGCAGGAGCAGCAGCTCTGAGAAACGTGGCCCAGAGATTATTTGAAAACTACC
AAACGCAATCTGAAGAAGTGAGAAAGAAGCAGGAGGACAGTAAACAATTACTCCAGGTTAACAAGCTT
GAAAAAGAACAGAAATTGAAACAACATGTTGAAAATCTGAATCAAGTTGCTGAAAAACTTGAAGAAAA
ACACAGTCAAATTACAGAATTGGAGAACCTTGTACAGAGAATGGAAAAGGAAAAGAGAACACTACTAG
AAAGAAAACTGTCTTTGGAAAACAAGCTACTGCAACTCAAATCCAGTGCTACATATGGAAAAAGTTGC
CAGGATCTTCAGAGGGAGATTTCCATTCTCCAGGAGCAGATCTCTCATCTGCAGTTTGTGATTCACTC
CCAACATCAGAACCTGCGCAGTGTCATCCAGGAGATGGAAGGATTAAAAAATAATTTAAAAGAACAAG
ACAAAAGAATTGAAAATCTCAGAGAAAAGGTTAACATACTTGAAGCCCAGAATAAAGAACTAAAAACC
CAGGTAGCACTTTCATCTGAAACTCCTAGGACAAAGGTATCTAAGGCTGTCTCTACAAGTGAATTGAA
GACCGAAGGTGTTTCCCCTTATTTAATGTTGATTAGGTTACGGAAATGAACTGGCTGGATGAAGATCT
GATTTAGAAAGACTGCGTGAGTCTTATTTATTCTCTGAAACACAGCCCAAGTTTCATGTTAAAATGGC
AAAATGCCATTATTTAAATGGAACTTATTACATACCAATGGCTTTGCAAGAAGATGACATTTCAGAAA
ATCAAACAAATCTATATTTAATGGATGGACTCTTCAAAACTTACCAAATAGTTGAAGAAACCAGGTGC
CTTCTCATGATGGAAGACAGATTCTGCTTTAAATTAAAAAAAAAAAAATCTGAATCTTGTTTTCAGAT
TTTTTTTTCTACTGG

| | | | |
|---|---|---|---|
| NOV15a, CG51992-05 | SEQ ID NO: 420 | 288 aa | MW at 33581.0kD |
| Protein Sequence | | | |

MTTVTVTTEIPPRDKMEDNSALYESTSAHIIEETEYVKKIRTTLQKIRTQMFKDEIRHDSTNHKLDAK
LQASREAGAAALRNVAQRLFENYQTQSEEVRKKQEDSKQLLQVNKLEKEQKLKQHVENLNQVAEKLEE
KHSQITELENLVQRMEKEKRTLLERKLSLENKLLQLKSSATYGKSCQDLQREISILQEQISHLQFVIH
SQHQNLRSVIQEMEGLKNNLKEQDKRIENLREKVNILEAQNKELKTQVALSSETPRTKVSKAVSTSEL
KTEGVSPYLMLIRLRK

| | | |
|---|---|---|
| NOV15b, CG51992-01 | SEQ ID NO: 421 | 705 bp |
| DNA Sequence | ORF Start: at 3 | ORF Stop: TGA at 423 |

TACAAAGAATGGAAAAGGAAAAGAGAACACTACTAGAAAGAAAACTGTCTTTGGAAAACAAGCTACTG
CAACTCAAATCCAGTGCTACATATGGAAAAAGTTGCCAGGATCTTCAGAGGGAGATTTCCATTCTCCA
GGAGCAGATCTCTCATCTGCAGTTTGTGATTCACTCCCAACATCAGAACCTGCGCAGTGTCATCCAGG
AGATGGAAGGATTAAAAAATAATTTAAAAGAACAAGACAAAAGAATTGAAAATCTCAGAGAAAAGGTT
AACATACTTGAAGCCCAGAATAAAGAACTAAAAACCCAGGTAGCACTTTCATCTGAAACTCCTAGGAC
AAAGGTATCTAAGGCTGTCTCTACAAGTGAATTGAAGACCGAAGGTGTTTCCCCTTATTTAATGTTGA
TTAGGTTACGGAAATGAACTGGCTGGATGAAGATCTGATTTAGAAAGACTGCGTGAGTCTTATTTATT
CTCTGAAACACACCCCAAGTTTCATGTTAAAATGGCAAAATGCCATTATTTAAATGGAACTTATTACA
TACCAATGGCTTTGCAAGAAGATGACATTTCAGAAAATCAAACAAATCTATATTTAATGGATGGACTC
TTCAAAACTTACCAAATAGTTGAAGAAACCAGGTGCCTTCTCATGATGGAAGACAGATTCTGCTTTAA
ATTAAAAAAAAAAAAATCTGAAAAA

| | | | |
|---|---|---|---|
| NOV15b, CG51992-01 | SEQ ID NO: 422 | 140 aa | MW at 16324.7kD |
| Protein Sequence | | | |

QRMEKEKRTLLERKLSLENKLLQLKSSATYGKSCQDLQREISILQEQISHLQFVIHSQHQNLRSVIQE
MEGLKNNLKEQDKRIENLREKVNILEAQNKELKTQVALSSETPRTKVSKAVSTSELKTEGVSPYLMLI
RLRK

| | | |
|---|---|---|
| NOV15c, CG51992-02 | SEQ ID NO: 423 | 1080 bp |
| DNA Sequence | ORF Start: ATG at 45 | ORF Stop: TGA at 798 |

GTTTAAAGATGAAATGAGACATGACAGTACAAATCACAAACTAGATGCAAAGTTTGGATTTGCTTATG
AAAAAGATAAAAGGAAAAGACCTACAGCTCTTAGAAATGAACAAAGAGAATGAAGTATTGAAAATCAA
GCTGCAAGCCTCCAGAGAAGCAGGAGCAGCAGCTCTGAGAAACGTGGCCCAGAGATTATTTGAAAACT
ACCAAACGCAATCTGAAGAAGTGAGAAAGAAGCAGGAGGGCAGTAAACAATTACTCCAGGTTAACAAG
CTTGAAAAAGAACAGAAATTGAAACAACATGTTGAAAATCTGAATCAAGTTGCTGAAAAACTTGAAGA
AAAACACAGTCAAATTACAGAATTGGAGAACCTTGTACAGAGAATGGAAAAGGAAAAGAGAACACTAC
TAGAAAGAAAACTGTCTTTGGAAAACAAGCTACTGCAACTCAAATCCAGTGCTACATATGGAAAAAGT
TGCCAGGATCTTCAGAGGGAGATTTCCATTCTCCAGGAGCAGATCTCTCATCTGCAGTTTGTGATTCA
CTCCCAACATCAGAACCTGCGCAGTGTCATCCAGGAGATGGAAGGATTAAAAAATAATTTAAAAGAAC
AAGACAAAAGAATTGAAAATCTCAGAGAAAAGGTTAACATACTTGAAGCCCAGAATAAAGAACTAAAA
ACCCAGGTAGCACTTTCATCTGAAACTCCTAGGACAAAGGTATCTAAGGCTGTCTCTACAAGTGAATT
GAAGACCGAAGGTGTTTCCCCTTATTTAATGTTGATTAGGTTACGGAAATGAACTGGCTGGATGAAGA
TCTGATTTAGAAAGACTGCGTGAGTCTTATTTATTCTCTGAAACACAGCCCAAGTTTCATGTTAAAAT
GGCAAAATGCCATTATTTAAATGGAACTTATTACATACCAATGGCTTTGCAAGAAGATGACATTTCAG
AAAATCAAACAAATCTATATTTAATGGATGGACTCTTCAAAACTTACCAAATAGTTGAAGAAACCAGG
TGCCTTCTCATGATGGAAGACAGATTCTGCTTTAAATTAAAAAAAAAAAAATCTGAAAAA

| | | | |
|---|---|---|---|
| NOV15c, CG51992-02 | SEQ ID NO: 424 | 251 aa | MW at 29229.5kD |
| Protein Sequence | | | |

MQSLDLLMKKIKGKDLQLLEMNKENEVLKIKLQASREAGAAALRNVAQRLFENYQTQSEEVRKKQEGS
KQLLQVNKLEKEQKLKQHVENLNQVAEKLEEKHSQITELENLVQRMEKEKRTLLERKLSLENKLLQLK
SSATYGKSCQDLQREISILQEQISHLQFVIHSQHQNLRSVIQEMEGLKNNLKEQDKRIENLREKVNIL
EAQNKELKTQVALSSETPRTKVSKAVSTSELKTEGVSPYLMLIRLRK

| | | |
|---|---|---|
| NOV15d, CG51992-03 | SEQ ID NO: 425 | 1442 bp |
| DNA Sequence | ORF Start: ATG at 155 | ORF Stop: TGA at 1160 |

GCCCAGGGGAGGAGCAGCACCGGGACCCCGCGTCGGCTGGGCGCCCCACAAGGGAAGCCAGTCTTAAT
ATGATGGAAACATCTCTGAACTTCTAAAAGACCAAGGTTGGCGTTTTAGCTCTATTAATTTTACTTCG
TCTTGGCCAGAATTCACAATGACAACAGTGACAGTGACCACAGAAATTCCCCCAAGGGATAAGATGGA
AGATAATTCTGCCTTGTATGAGTCTACGTCCGCTCACATTATTGAAGAAACCGAGTATGTGAAAAAGA
TTCGAACTACTCTGCAAAAGATCAGGACCCAGATGTTTAAAGATGAAATAAGACATGACAGTACAAAT
CACAAACTAGATGCAAAGCACTGTGGAAACCTTCAACAGGGCTCTGATTCTGAAATGGATCCTTCTTG
TTGCAGTTTGGATTTGCTTATGAAAAAGATAAAAGGAAAAGACCTACAGCTCTTAGAAATGAACAAAG
AGAATGAAGTATTGAAAATCAAGCTGCAAGCCTCCAGAGAAGCAGGAGCAGCAGCTCTGAGAAACGTG
GCCCAGAGATTATTTGAAAACTACCAAACGCAATCTGAAGAAGTGAGAAAGAAGCAGGAGGACAGTAA
ACAATTACTCCAGGTTAACAAGCTTGAAAAAGAACAGAAATTGAAACAACATGTTGAAAATCTGAATC
AAGTTGCTGAAAAACTTGAAGAAAAACACAGTCAAATTACAGAATTGGAGAACCTTGTACAGAGAATG
GAAAAGGAAAAGAGAACACTACTAGAAAGAAAACTGTCTTTGGAAAACAAGCTACTGCAACTCAAATC
CAGTGCTACATATGGAAAAAGTTGCCAGGATCTTCAGAGGGAGATTTCCATTCTCCAGGAGCAGATCT
CTCATCTGCAGTTTGTGATTCACTCCCAACATCAGAACCTGCGCAGTGTCATCCAGGAGATGGAAGGA
TTAAAAAATAATTTAAAAGAACAAGACAAAAGAATTGAAAATCTCAGAGAAAAGGTTAACATACTTGA
AGCCCAGAATAAAGAACTAAAAACCCAGGTAGCACTTTCATCTGAAACTCCTAGGACAAAGGTATCTA
AGGCTGTCTCTACAAGTGAATTGAAGACCGAAGGTGTTTCCCCTTATTTAATGTTGATTAGGTTACGG
AAATGAACTGGCTGGATGAAGATCTGATTTAGAAAGACTGCGTGAGTCTTATTTATTCTCTGAAACAC
AGCCCAAGTTTCATGTTAAAATGGCAAAATGCCATTATTTAAATGGAACTTATTACATACCAATGGCT
TTGCAAGAAGATGACATTTCAGAAAATCAAACAAATCTATATTTAATGGATGGACTCTTCAAAACTTA
CCAAATAGTTGAAGAAACCAGGTGCCTTCTCATGATGGAAGACAGATTCTGCTTTAAATTAAAAAAAA
AAAAATCTGAAAAA

| | | | |
|---|---|---|---|
| NOV15d, CG51992-03 | SEQ ID NO: 426 | 335 aa | MW at 38869.2kD |
| Protein Sequence | | | |

MTTVTVTTEIPPRDKMEDNSALYESTSAHIIEETEYVKKIRTTLQKIRTQMFKDEIRHDSTNHKLDAK
HCGNLQQGSDSEMDPSCCSLDLLMKKIKGKDLQLLENNKENEVLKIKLQASREAGAAALRNVAQRLFE
NYQTQSEEVRKKQEDSKQLLQVNKLEKEQKLKQHVENLNQVAEKLEEKHSQITELENLVQRMEKEKRT
LLERKLSLENKLLQLKSSATYGKSCQDLQREISILQEQISHLQFVIHSQHQNLRSVIQEMEGLKNNLK
EQDKRIENLREKVNILEAQNKELKTQVALSSETPRTKVSKAVSTSELKTEGVSPYLMLIRLRK

| | | |
|---|---|---|
| NOV15e, CG51992-04 | SEQ ID NO: 427 | 1482bp |
| DNA Sequence | ORF Start: ATG at 195 | ORF Stop: TGA at 1200 |

GTGTGTGGGTGTCCAGGTGCCTTTCCAGCGGCTTCCCCAGTGGAGTTCCTGGCATCAAGGACATTTCC
TGTAAAAGGGTCCTTGTTGAAGAGGGAAGCCAGTCTTAATATGATGGAAACATCTCTGAACTTCTAAA
AGACCAAGGTTGGCGTTTTAGCTCTATTAATTTTACTTCGTCTTGGCCAGAATTCACAATGACAACAG
TGGCAGTGACCACAGAAATTCCCCCAAGGGATAAGATGGAAGATAATTCTGCCTTGTATGAGTCTACG
TCCGCTCACATTATTGAAGAAACCGAGTATGTGAAAAAGATTCGAACTACTCTGCAAAAGATCAGGAC
CCAGATGTTTAAAGATGAAATAAGACATGACAGTACAAATCACAAACTAGATGCAAAGCACTGTGGAA
ACCTTCAACAGGGCTCTGATTCTGAAATGGATCCTTCTTGTTGCAGTTTGGATTTGCTTATGAAAAAG
ATAAAAGGAAAAGACCTACAGCTCTTAGAAATGAACAAAGAGAATGAAGTATTGAAAATCAAGCTGCA
AGCCTCCAGAGAAGCAGGAGCAGCAGCTCTGAGAAACGTGGCCCAGAGATTATTTGAAAACTACCAAA
CGCAATCTGAAGAAGTGAGAAAGAAGCAGGAGGACAGTAAACAATTACTCCAGGTTAACAAGCTTGAA
AAAGAACAGAAATTGAAACAACATGTTGAAAATCTGAATCAAGTTGCTGAAAAACTTGAAGAAAAACA
CAGTCAAATTACAGAATTGGAGAACCTTGTACAGAGAATGGAAAAGGAAAAGAGAACACTACTAGAAA
GAAAACTGTCTTTGGAAAACAAGCTACTGCAACTCAAATCCAGTGCTACATATGGAAAAAGTTGCCAG
GATCTTCAGAGGGAGATTTCCATTCTCCAGGAGCAGATCTCTCATCTGCAGTTTGTGATTCACTCCCA
ACATCAGAACCTGCGCAGTGTCATCCAGGAGATGGAAGGATTAAAAAATAATTTAAAAGAACAAGACA
AAAGAATTGAAAATCTCAGAGAAAAGGTTAACATACTTGAAGCCCAGAATAAAGAACTAAAAACCCAG
GTAGCACTTTCATCTGAAACTCCTAGGACAAAGGTATCTAAGGCTGTCTCTACAAGTGAATTGAAGAC
CGAAGGTGTTTCCCCTTATTTAATGTTGATTAGGTTACGGAAATGAACTGGCTGGATGAAGATCTGAT
TTAGAAAGACTGCGTGAGTCTTATTTATTCTCTGAAACACAGCCCAAGTTTCATGTTAAAATGGCAAA
ATGCCATTATTTAAATGGAACTTATTACATACCAATGGCTTTGCAAGAAGATGACATTTCAGAAAATC
AAACAAATCTATATTTAATGGATGGACTCTTCAAAACTTACCAAATAGTTGAAGAAACCAGGTGCCTT
+E,UNA CTCATGATGGAAGACAGATTCTGCTTTAAATTAAAAAAAAAAAAATCTGAAAAA

| | | | |
|---|---|---|---|
| NOV15e, CG51992-04 | SEQ ID NO: 428 | 335 aa | MW at 38839.1kD |
| Protein Sequence | | | |

MTTVAVTTEIPPRDKMEDNSALYESTSAHIIEETEYVKKIRTTLQKIRTQMFKDEIRHDSTNHKLDAK
HCGNLQQGSDSEMDPSCCSLDLLMKKIKGKDLQLLEMNKENEVLKIKLQASREAGAAALRINAQRLFE
NYQTQSEEVRKKQEDSKQLLQVNKLEKEQKLKQHVENLNQVAEKLEEKHSQITELENLVQRMEKEKRT
LLERKLSLENKLLQLKSSATYGKSCQDLQREISILQEQISHLQFVIHSQHQNLRSVIQEMEGLKNNLK
EQDKRIENLREKVNILEAQNKELKTQVALSSETPRTKVSKAVSTSELKTEGVSPYLMLIRLRK

| | | |
|---|---|---|
| NOV15f, SNP13382486 | SEQ ID NO: 429 | 1511 bp |
| of CG51992-05, | ORF Start: ATG at 339 | ORF Stop: TGA at 1203 |
| DNA Sequence | SNP Pos: 506 | SNP Change: A to G |

AGAGAAGGAGCTAAGAAGCCTGTGGCTACTGAGAAGAGGCCAGCTAAGTGATGCTGGCATGTAGGAAG
CAGGCACACTACTTGTGGATGGGGAGCATGGTGACTGGAGAGACCAGTGAGGGCAATTTGCATCCTTT
GAGGGCAATTTGGTCTCCAAAGAACAGAGTGATCTGGAGGGAGTGACAGCCAAGAGTCAGCCAGAGCA
TCTCAGCCATCCCAGGAATCAGAGAAATCCAGGGAAGCCAGTCTTAATATGATGGAAACATCTCTGAA
CTTCTAAAAGACCAAGGTTGGCGTTTTAGCTCTATTAATTTTACTTCGTCTTGGCCAGAATTCACAAT
GACAACAGTGACAGTGACCACAGAAATTCCCCCAAGGGATAAGATGGAAGATAATTCTGCCTTGTATG
AGTCTACGTCCGCTCACATTATTGAAAGAAACCGAGTATGTGAAAAAGATTCGAACTACTCTGCAAAG
ATCAGGACCCAGATGTTTAAAGATGAAATGAGACATGACAGTACAAATCACAAACTAGATGCAAAGCT
GCAAGCCTCCAGAGAAGCAGGAGCAGCAGCTCTGAGAAACGTGGCCCAGAGATTATTTGAAAACTACC
AAACGCAATCTGAAGAAGTGAGAAAGAAGCAGGAGGACAGTAAACAATTACTCCAGGTTAACAAGCTT
GAAAAAGAACAGAAATTGAAACAACATGTTGAAAATCTGAATCAAGTTGCTGAAAAACTTGAAGAAAA
ACACAGTCAAATTACAGAATTGGAGAACCTTGTACAGAGAATGGAAAAGGAAAAGAGAACACTACTAG
AAAGAAAACTGTCTTTGGAAAACAAGCTACTGCAACTCAAATCCAGTGCTACATATGGAAAAAGTTGC
CAGGATCTTCAGAGGGAGATTTCCATTCTCCAGGAGCAGATCTCTCATCTGCAGTTTGTGATTCACTC
CCAACATCAGAACCTGCGCAGTGTCATCCAGGAGATGGAAGGATTAAAAAATAATTTAAAAGAACAAG
ACAAAAGAATTGAAAATCTCAGAGAAAAGGTTAACATACTTGAAGCCCAGAATAAAGAACTAAAAACC
CAGGTAGCACTTTCATCTGAAACTCCTAGGACAAAGGTATCTAAGGCTGTCTCTACAAGTGAATTGAA
GACCGAAGGTGTTTCCCCTTATTTAATGTTGATTAGGTTACGGAAATGAACTGGCTGGATGAAGATCT
GATTTAGAAAGACTGCGTGAGTCTTATTTATTCTCTGAAACACAGCCCAAGTTTCATGTTAAAATGGC
AAAATGCCATTATTTAAATGGAACTTATTACATACCAATGGCTTTGCAAGAAGATGACATTTCAGAAA
ATCAAACAAATCTATATTTAATGGATGGACTCTTCAAAACTTACCAAATAGTTGAAGAAACCAGGTGC
CTTCTCATGATGGAAGACAGATTCTGCTTTAAATTAAAAAAAAAAAAATCTGAATCTTGTTTTCAGAT
TTTTTTTTCTACTGG

| | | | |
|---|---|---|---|
| NOV15f, SNP13382486 | SEQ ID NO: 430 | 288 aa | MW at 33599.1kD |
| of CG51992-05, | SNP Pos: 56 | | SNP Change: |
| Protein Sequence | | | Ile to Met |

MTTVTVTTEIPPRDKMEDNSALYESTSAHIIEETEYVKKIRTTLQKIRTQMFKDEMRHDSTNHKLDAK
LQASREAGAAALRNVAQRLFENYQTQSEEVRKKQEDSKQLLQVNKLEKEQKLKQHVENLNQVAEKLEE
KHSQITELENLVQRMEKEKRTLLERKLSLENKLLQLKSSATYGKSCQDLQREISILQEQISHLQFVIH
SQHQNLRSVIQEMEGLKNNLKEQDKRIENLREKVNILEAQNKELKTQVALSSETPRTKVSKAVSTSEL
KTEGVSPYLMLIRLRK

| | | |
|---|---|---|
| NOV15g, SNP13382487 | SEQ ID NO: 431 | 1511 bp |
| of CG51992-05, | ORF Start: ATG at 339 | ORF Stop: TGA at 1203 |
| DNA Sequence | SNP Pos: 1360 | SNP Change: A to G |

AGAGAAGGAGCTAAGAAGCCTGTGGCTACTGAGAAGAGGCCAGCTAAGTGATGCTGGCATGTAGGAAC
CAGGCACACTACTTGTGGATGGGGAGCATGGTGACTGGAGAGACCAGTGAGGGCAATTTGCATCCTTT
GAGGGCAATTTGGTCTCCAAAGAACAGAGTGATCTGGAGGGAGTGACAGCCAAGAGTCAGCCAGAGCA
TCTCAGCCATCCCAGGAATCAGAGAAATCCAGGGAAGCCAGTCTTAATATGATGGAAACATCTCTGAA
CTTCTAAAAGACCAAGGTTGGCGTTTTAGCTCTATTAATTTTACTTCGTCTTGGCCAGAATTCACAAT
GACAACAGTGACAGTGACCACAGAAATTCCCCCAAGGGATAAGATGGAAGATAATTCTGCCTTGTATG
AGTCTACGTCCGCTCACATTATTGAAGAAACCGAGTATGTGAAAAAGATTCGAACTACTCTGCAAAAG
ATCAGGACCCAGATGTTTAAAGATGAAATAAGACATGACAGTACAAATCACAAACTAGATGCAAAGCT
GCAAGCCTCCAGAGAAGCAGGAGCAGCAGCTCTGAGAAACGTGGCCCAGAGATTATTTGAAAACTACC
AAACGCAATCTGAAGAAGTGAGAAAGAAGCAGGAGGACAGTAAACAATTACTCCAGGTTAACAAGCTT
GAAAAAGAACAGAAATTGAAACAACATGTTGAAAATCTGAATCAAGTTGCTGAAAAACTTGAAGAAAA
ACACAGTCAAATTACAGAATTGGAGAACCTTGTACAGAGAATGGAAAAGGAAAAGAGAACACTACTAG
AAAGAAAACTGTCTTTGGAAAACAAGCTACTGCAACTCAAATCCAGTGCTACATATGGAAAAAGTTGC
CAGGATCTTCAGAGGGAGATTTCCATTCTCCAGGAGCAGATCTCTCATCTGCAGTTTGTGATTCACTC
CCAACATCAGAACCTGCGCAGTGTCATCCAGGAGATGGAAGGATTAAAAAATAATTTAAAAGAACAAG
ACAAAAGAATTGAAAATCTCAGAGAAAAGGTTAACATACTTGAAGCCCAGAATAAAGAACTAAAAACC
CAGGTAGCACTTTCATCTGAAACTCCTAGGACAAGGTATCTAAAGGCTGTCTCTACAAGTGAATTGAA
GACCGAAGGTGTTTCCCCTTATTTAATGTTGATTAGGTTACGGAAATGAACTGGCTGGATGAAGATCT
GATTTAGAAAGACTGCGTGAGTCTTATTTATTCTCTGAAACACAGCCCAAGTTTCATGTTAAAATGGC
AAAATGCCATTATTTAAATGGAACTTATTACATACCAATGGCTTTGCAAGAAGATGACATTTCAGAAG
ATCAAACAAATCTATATTTAATGGATGGACTCTTCAAAACTTACCAAATAGTTGAAGAAACCAGGTGC
CTTCTCATGATGGAAGACAGATTCTGCTTTAAATTAAAAAAAAAAAAATCTGAATCTTGTTTTCAGAT
TTTTTTTTCTACTGG

| | | | |
|---|---|---|---|
| NOV15g, SNP13382487 | SEQ ID NO: 432 | 1288 aa | MW at 33581.0kD |
| of CG51992-05, | | | SNP Change: |
| Protein Sequence | | | no change |

MTTVTVTTEIPPRDKMEDNSALYESTSAHIIEETEYVKKIRTTLQKIRTQMFKDEIRHDSTNBKLDAK
LQASREAGAAALRNVAQRLFENYQTQSEEVRKKQEDSKQLLQVNKLEKEQKLKQHVENLNQVAEKLEE
KHSQITELENLVQRMEKEKRTLLERKLSLENKLLQLKSSATYGKSCQDLQREISILQEQISHLQFVIH
SQHQNLRSVIQEMEGLKNNLKEQDKRIENLREKVNILEAQNKELKTQVALSSETPRTKVSKAVSTSEL
KTEGVSPYLMLIRLRK

A ClustalW comparison of the above protein sequences yields the following sequence alignment shown in Table 15B.

TABLE 15B
Comparison of the NOV15 protein sequences.

| | |
|---|---|
| NOV15a | MTTVTVTTEIPPRDKMEDNSALYESTSAHIIEETEYVKKIRTTLQKIRTQMFKDEIRHDS |
| NOV15b | ------------------------------------------------------------ |
| NOV15c | ------------------------------------MQSLDLLMKKIKGKDLQ-LLEMNK |
| NOV15d | MTTVTVTTEIPPRDKMEDNSALYESTSAHIIEETEYVKKIRTTLQKIRTQMFKDEIRHDS |
| NOV15e | MTTVAVTTEIPPRDKMEDNSALYESTSAHIIEETEYVKKIRTTLQKIRTQMFKDEIRHDS |
| NOV15a | TNHKLDAK-----------------------------------------------LQASR |
| NOV15b | ------------------------------------------------------------ |
| NOV15c | ENEVLKIK-----------------------------------------------LQASR |
| NOV15d | TNHKLDAKHCGNLQQGSDSEMDPSCCSLDLLMKKIKGKDLQLLEMNKENEVLKIKLQASR |
| NOV15e | TNHKLDAKHCGNLQQGSDSEMDPSCCSLDLLMKKIKGKDLQLLEMNKENEVLKIKLQASR |
| NOV15a | EAGAAALRNVAQRLFENYQTQSEEVRKKQEDSKQLLQVNKLEKEQKLKQHVENLNQVAEK |
| NOV15b | ------------------------------------------------------------ |
| NOV15c | EAGAAALRNVAQRLFENYQTQSEEVRKKQEGSKQLLQVNKLEKEQKLKQHVENLNQVAEK |
| NOV15d | EAGAAALRNVAQRLFENYQTQSEEVRKKQEDSKQLLQVNKLEKEQKLKQHVENLNQVAEK |
| NOV15e | EAGAAALRNVAQRLFENYQTQSEEVRKKQEDSKQLLQVNKLEKEQKLKQHVENLNQVAEK |
| NOV15a | LEEKHSQITELENLVQRMEKEKRTLLERYLSLENKLLQLKSSATYGKSCQDLQREISILQ |
| NOV15b | ---------------QRMEKEKRTLLERKLSLENKLLQLKSSATYGKSCQDLQREISILQ |
| NOV15c | LEEKHSQITELENLVQRMEKEKRTLLERKLSLENKLLQLKSSATYGKSCQDLQREISILQ |
| NOV15d | LEEKHSQITELENLVQRMEKEKRTLLERKLSLENKLLQLKSSATYGKSCQDLQREISILQ |
| NOV15e | LEEKHSQITELENLVQRMEKEKRTLLERKLSLENKLLQLKSSATYGKSCQDLQREISILQ |
| NOV15a | EQISHLQFVIHSQHQNLRSVIQEMEGLKNNLKEQDKRIENLREKVNILEAQNKELKTQVA |
| NOV15b | EQISHLQFVIHSQHQNLRSVIQEMEGLKNNLKEQDKRIENLREKVNILEAQNKELKTQVA |
| NOV15c | EQISHLQFVIHSQHQNLRSVIQEMEGLKNNLKEQDKRIENLREKVNILEAQNKELKTQVA |
| NOV15d | EQISHLQFVIHSQHQNLRSVIQEMEGLKNNLKEQDKRIENLREKVNILEAQNKELKTQVA |
| NOV15e | EQISHLQFVIHSQHQNLRSVIQEMEGLKNNLKEQDKRIENLREKVNILEAQNKELKTQVA |
| NOV15a | LSSETPRTKVSKAVSTSELKTEGVSPYLMLIRLRK |
| NOV15b | LSSETPRTKVSKAVSTSELKTEGVSPYLMLIRLRK |
| NOV15c | LSSETPRTKVSKAVSTSELKTEGVSPYLMLIRLRK |
| NOV15d | LSSETPRTKVSKAVSTSELKTEGVSPYLMLIRLRK |
| NOV15e | LSSETPRTKVSKAVSTSELKTEGVSPYLMLIRLRK |
| NOV15a | (SEQ ID NO: 420) |
| NOV15b | (SEQ ID NO: 422) |
| NOV15c | (SEQ ID NO: 424) |
| NOV15d | (SEQ ID NO: 426) |
| NOV15e | (SEQ ID NO: 428) |

Further analysis of the NOV15a protein yielded the following properties shown in Table 15C.

TABLE 15C
Protein Sequence Properties NOV15a

| | |
|---|---|
| SignalP analysis: | No Known Signal Sequence Predicted |
| PSORT II analysis: | |

| PSG: | a new signal peptide prediction method | |
|---|---|---|
| | N-region: | length 9; pos. chg 0; neg. chg 1 |
| | H-region: | length 3; peak value 0.00 |
| | PSG score: | −4.40 |

| | |
|---|---|
| GvH: | von Heijne's method for signal seq. recognition |
| | GvH score (threshold: −2.1): −11.91 |
| | possible cleavage site: between 49 and 50 |

>>> Seems to have no N-terminal signal peptide

| ALOM: | Klein et al's method for TM region allocation | | | |
|---|---|---|---|---|
| | Init position for calculation: 1 | | | |
| | Tentative number of TMS(s) for the threshold 0.5: 0 | | | |
| | number of TMS(s) . . . fixed | | | |
| | PERIPHERAL Likelihood = 9.07 (at 189) | | | |
| | ALOM score: 9.07 (number of TMSs: 0) | | | |
| MITDISC: | discrimination of mitochondrial targeting seq | | | |
| | R content: | 1 | Hyd Moment(75): | 1.56 |
| | Hyd Moment(95): | 1.30 | G content: | 0 |
| | D/E content: | 2 | S/T content: | 5 |

| | |
|---|---|
| | Score: −5.82 |
| Gavel: | prediction of cleavage sites for mitochondrial preseq |
| | cleavage site motif not found |

NUCDISC: discrimination of nuclear localization signals

| | |
|---|---|
| | pat4: none |
| | pat7: none |
| | bipartite: none |
| | content of basic residues: 17.4% |
| | NLS Score: −0.47 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 94.1 |
| COIL: | Lupas's algorithm to detect coiled-coil regions |
| 76 A | 0.56 |
| 77 A | 0.56 |
| 78 A | 0.56 |
| 79 L | 0.56 |
| 80 R | 0.56 |
| 81 N | 0.59 |
| 82 V | 0.67 |
| 83 A | 0.67 |
| 84 Q | 0.67 |
| 85 R | 0.67 |
| 86 L | 0.67 |
| 87 F | 0.67 |
| 88 E | 0.67 |
| 89 N | 0.67 |
| 90 Y | 0.67 |
| 91 Q | 0.67 |
| 92 T | 0.67 |
| 93 Q | 0.80 |
| 94 S | 0.80 |
| 95 E | 0.80 |
| 96 E | 0.80 |
| 97 V | 0.80 |
| 98 R | 0.80 |
| 99 K | 0.81 |
| 100 K | 0.81 |
| 101 Q | 0.81 |
| 102 E | 0.81 |
| 103 D | 0.81 |
| 104 S | 0.81 |
| 105 K | 0.81 |
| 106 Q | 0.81 |
| 107 L | 0.86 |
| 108 L | 0.98 |
| 109 Q | 0.98 |
| 110 V | 0.98 |
| 111 N | 0.98 |
| 112 K | 0.98 |
| 113 L | 0.98 |
| 114 E | 0.98 |
| 115 K | 0.98 |
| 116 E | 0.99 |
| 117 Q | 0.99 |
| 118 K | 0.99 |
| 119 L | 0.99 |
| 120 K | 0.99 |
| 121 Q | 0.99 |
| 122 H | 0.99 |
| 123 V | 0.99 |
| 124 E | 1.00 |
| 125 N | 1.00 |
| 126 L | 1.00 |
| 127 N | 1.00 |
| 128 Q | 1.00 |
| 129 V | 1.00 |
| 130 A | 1.00 |
| 131 E | 1.00 |
| 132 K | 1.00 |
| 133 L | 1.00 |
| 134 E | 1.00 |
| 135 E | 1.00 |
| 136 K | 1.00 |
| 137 H | 1.00 |
| 138 S | 1.00 |
| 139 Q | 1.00 |
| 140 I | 1.00 |
| 141 T | 1.00 |
| 142 E | 1.00 |
| 143 L | 1.00 |
| 144 E | 1.00 |
| 145 N | 1.00 |
| 146 L | 1.00 |
| 147 V | 1.00 |
| 148 Q | 1.00 |
| 149 R | 1.00 |
| 150 M | 1.00 |
| 151 E | 1.00 |
| 152 K | 1.00 |
| 153 E | 1.00 |
| 154 K | 1.00 |
| 155 R | 1.00 |
| 156 T | 1.00 |
| 157 L | 1.00 |
| 158 L | 1.00 |
| 159 E | 1.00 |
| 160 R | 1.00 |
| 161 K | 1.00 |
| 162 L | 1.00 |
| 163 S | 1.00 |
| 164 L | 1.00 |
| 165 E | 1.00 |
| 166 N | 1.00 |
| 167 K | 1.00 |
| 168 L | 1.00 |
| 169 L | 1.00 |
| 170 Q | 1.00 |
| 171 L | 1.00 |
| 172 K | 1.00 |
| 173 S | 1.00 |
| 174 S | 1.00 |
| 175 A | 1.00 |
| 176 T | 0.99 |
| 177 Y | 0.88 |
| 178 G | 0.83 |
| 179 K | 0.83 |
| 180 S | 0.83 |
| 181 C | 0.83 |
| 182 Q | 0.83 |
| 183 D | 0.83 |
| 184 L | 0.83 |
| 185 Q | 0.83 |
| 186 R | 0.83 |
| 187 E | 0.83 |
| 188 I | 0.83 |
| 189 S | 0.83 |
| 190 I | 0.83 |
| 191 L | 0.83 |
| 192 Q | 0.83 |
| 193 E | 0.83 |
| 194 Q | 0.83 |
| 195 I | 0.83 |
| 196 S | 0.83 |
| 197 H | 0.83 |
| 198 L | 0.83 |
| 199 Q | 0.83 |
| 200 F | 0.62 |
| 201 V | 0.62 |
| 202 I | 0.87 |
| 203 H | 0.87 |
| 204 S | 0.99 |
| 205 Q | 0.99 |
| 206 H | 1.00 |
| 207 Q | 1.00 |
| 208 N | 1.00 |
| 209 L | 1.00 |
| 210 R | 1.00 |
| 211 S | 1.00 |
| 212 V | 1.00 |
| 213 I | 1.00 |
| 214 Q | 1.00 |
| 215 E | 1.00 |
| 216 M | 1.00 |
| 217 E | 1.00 |
| 218 G | 1.00 |
| 219 L | 1.00 |
| 220 K | 1.00 |
| 221 N | 1.00 |
| 222 N | 1.00 |
| 223 L | 1.00 |
| 224 K | 1.00 |
| 225 E | 1.00 |
| 226 Q | 1.00 |
| 227 D | 1.00 |
| 228 K | 1.00 |
| 229 R | 1.00 |
| 230 I | 1.00 |
| 231 E | 1.00 |
| 232 N | 1.00 |
| 233 L | 1.00 |
| 234 R | 1.00 |
| 235 E | 1.00 |
| 236 K | 1.00 |
| 237 V | 1.00 |
| 238 N | 1.00 |
| 239 I | 1.00 |
| 240 L | 1.00 |
| 241 E | 1.00 |
| 242 A | 1.00 |
| 243 Q | 1.00 |
| 244 N | 1.00 |
| 245 K | 1.00 |
| 246 E | 1.00 |
| 247 L | 1.00 |
| 248 K | 1.00 |
| 249 T | 1.00 |
| 250 Q | 1.00 |
| 251 V | 1.00 |
| 252 A | 1.00 |
| 253 L | 1.00 |
| 254 S | 1.00 |
| 255 S | 1.00 |
| 256 E | 1.00 |
| 257 T | 1.00 |
| | total: 182 residues |

Final Results (k = 9/23):

```
69.6%: nuclear
13.0%: cytoplasmic
  8.7%: peroxisomal
  4.3%: cytoskeletal
  4.3%: mitochondrial
```

>> prediction for CG51992-05 is nuc (k = 23)

A search of the NOV15a protein against the Geneseq database, a proprietary se that contains sequences published in patents and patent publication, yielded homologous proteins shown in Table 15D.

TABLE 15D
Geneseq Results for NOV15a

| | | NOV15a | Identities/ | | |
|---|---|---|---|---|---|
| | | Residues/ | Similarities for | | |
| Geneseq | Protein/Organism/Length | Match | the Matched | | Expect |
| Identifier | [Patent #, Date] | Residues | Region | | Value |
| ABB77426 | Human tumour marker protein se57-1 - | 1 . . . 288 | 288/335 | (85%) | e−151 |
| | Homo sapiens, 335 aa. [WO200238803- | 1 . . . 335 | 288/335 | (85%) | |
| | A2, 16-MAY-2002] | | | | |
| AAU08663 | Human NOV7 protein - Homo sapiens, | 1 . . . 288 | 288/335 | (85%) | e−151 |
| | 335 aa. [WO200168851-A2, 20-SEP- | 1 . . . 335 | 288/335 | (85%) | |
| | 2001] | | | | |
| AAU08662 | Human NOV6 protein - Homo sapiens, | 1 . . . 288 | 287/335 | (85%) | e−150 |
| | 335 aa. [WO200168851-A2, 20-SEP- | 1 . . . 335 | 287/335 | (85%) | |
| | 2001] | | | | |
| AAU08661 | Human NOV5 protein - Homo sapiens, | 62 . . . 288 | 222/227 | (97%) | e−117 |
| | 251 aa. [WO200168851-A2, 20-SEP- | 25 . . . 251 | 222/227 | (97%) | |
| | 2001] | | | | |
| ABG79634 | Human novel secreted protein SECP10, | 43 . . . 258 | 67/221 | (30%) | 1e−21 |
| | Incyte ID No. 7939381CD1 - Homo | 202 . . . 415 | 125/221 | (56%) | |
| | sapiens, 466 aa. [WO200262841-A2, | | | | |
| | 15-AUG-2002] | | | | |

In a BLAST search of public sequence databases, the NOV15a protein was found to have homology to the proteins shown in the BLASTP data in Table 15E.

TABLE 15E
Public BLASTP Results for NOV15a

| | | NOV15a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| Q9H2F9 | CTCL tumor antigen se57-1 - | 1 . . . 288 | 288/335 | (85%) | e−150 |
| | Homo sapiens (Human), 335 aa. | 1 . . . 335 | 288/335 | (85%) | |
| Q8BVC4 | Similar to CTCL tumor antigen | 1 . . . 288 | 213/333 | (63%) | e−106 |
| | SE57-1 - Mus musculus (Mouse), | 1 . . . 333 | 241/333 | (71%) | |
| | 333 aa. | | | | |
| AAH46343 | Hypothetical protein - Mus | 1 . . . 155 | 102/200 | (51%) | 3e−43 |
| | musculus (Mouse), 242 aa. | 1 . . . 200 | 120/200 | (60%) | |
| Q96NF5 | Hypothetical protein FLJ30973 - | 43 . . . 258 | 67/221 | (30%) | 5e−21 |
| | Homo sapiens (Human), 466 aa. | 202 . . . 415 | 125/221 | (56%) | |
| A40809 | enamelin, 44K - bovine, 389 aa. | 22 . . . 261 | 61/275 | (22%) | 6e−15 |
| | | 39 . . . 309 | 131/275 | (47%) | |

PFam analysis predicts that the NOV15a protein contains the domains shown in the Table 15F.

TABLE 15F
Domain Analysis of NOV15a

| | | | |
|---|---|---|---|
| Pfam | NOV15a | Identities/ | Expect |
| Domain | Match Region | Similarities | Value |
| | | for the | |
| | | Matched Region | |

### Example 16

The NOV16 clone was analyzed, and the nucleotide and encoded polypeptide sequences are shown in Table 16A.

TABLE 16A
NOV16 Sequence Analysis

| | | |
|---|---|---|
| NOV16a, CG52171-04 | SEQ ID NO: 433 | 2403 bp |
| DNA Sequence | ORF Start: ATG at 81 | ORF Stop: TGA at 2292 |

GGCACGAGAAGTTAAGCTGAAGACCGAAGCAAGAGCTGGTTCAGGTGGCAGCCACAGCAGCCTCAGGC
ACCTCAGCAACTATGCCCTCCTGCCCAGACTCTGATAATAGCTGGGTGCTTGCTGGCTCCGAGAGCCT
GCCAGTGGAGACACTGGGCCCGGCATCCAGGATGGACCCAGAATCTGAGAGAGCCCTGCAGGCCCCTC
ACAGCCCCTCCAAGACAGATGGGAAAGAATTAGCTGGGACCATGGATGGAGAAGGGACGCTCTTCCAG
ACTGAAAGCCCTCAGTCTGGCAGCATTCTAACAGAGGAGACTGAGGTCAAGGGCACCCTGGAAGGTGA
TGTTTGTGGTGTGGAGCCTCCTGGCCCAGGAGACACAGTAGTCCAGGGAGACCTGCAGGAGACCACCG
TGGTGACAGGCCTGGGACCAGACACACAGGACCTGGAAGGCCAGAGCCCTCCACAGAGCCTGCCTTCA
ACCCCCAAAGCAGCTTGGATCAGGGAGGAGGGCCGCTGCTCCAGCAGTGACGATGACACCGACGTGGA
CATGGAGGGTCTGCGGAGACGGCGGGGCCGGGAGGCCGGCCCACCTCAGCCCATGGTGCCCCTGGCTG
TGGAGAACCAGGCTGGGGGTGAGGGTGCAGGCGGGGAGCTGGGCATCTCCCTCAACATGTGCCTCCTT
GGGGCCCTGGTTCTGCTTGGCCTGGGGGTCCTCCTCTTCTCAGGTGGCCTCTCAGAGTCTGAGACTGG
GCCCATGGAGGAAGTGGAGCGGCAGGTCCTCCCAGACCCCGAGGTGCTGGAAGCTGTGGGGGACAGGC
AGGATGGGCTAAGGGAACAGCTGCAGGCCCCAGTGCCTCCTGACAGTGTCCCCAGCCTGCAAAACATG
GGTCTTCTGCTGGACAAGCTGGCCAAGGAGAACCAGGACATCCGGCTGCTGCAGGCCCAGCTGCAGGC
CCAAAAGGAAGAGCTTCAGAGCCTGATGCACCAGCCCAAAGGGCTAGAGGAGGAGAATGCCCAGCTCC
GGGGGGCTCTGCAGCAGGGCGAAGCCTTCCAGCGGGCTCTGGAGTCAGAGCTGCAGCAGCTGCGGGCC
CGGCTCCAGGGGCTGGAGGCCGACTGTGTCCGGGGCCCAGATGGGGTGTGCCTCAGTGGGGATAGAGG
CCCACAGGGTGACAAGGCCATCAGGGAGCAAGGCCCCAGGGAGCAGGAGCCAGAACTCAGCTTCCTGA
AGCAGAAGGAACAGCTGGAGGCTGAGGCACAGGCATTAAGGCAAGAGTTAGAGAGGCAGCGACGGCTG
CTGGGGTCTGTACAGCAGGATCTGGAGAGGAGCTTGCAGGATGCCAGCCGCGGGGACCCAGCTCATGC
TGGCTTGGCTGAGCTGGGCCACAGATTGGCCCAGAAACTGCAGGGCCTGGAGAACTGGGGCCAGGACC
CTGGGGTCTCTGCCAATGCCTCAAAGGCCTGGCACCAGAAGTCCCACTTCCAGAATTCTAGGGAGTGG
AGTGGAAAGGAAAAGTGGTGGGATGGGCAGAGAGACCGGAAGGCTGAGCACTGGAAACATAAGAAGGA
AGAATCTGGCCGGGAAAGGAAGAAGAACTGGGGAGGTCAGGAGGACAGGGAGCCAGCAGGAAGGTGGA
AGGAGGGCAGGCCAAGGGTGGAGGAGTCGGGGAGCAAGAAGGAGGGCAAGCGACAGGGCCCGAAGGAA
CCCCCAAGGAAAAGTGGTAGCTTCCACTCCTCTGGAGAAAAGCAGAAGCAACCTCGGTGGAGGGAAGG
GACTAAGGACAGCCATGACCCCCTGCCATCCTGGGCAGAGCTGTTGAGGCCCAAGTACCGGGCACCCC
AGGGCTGCTCAGGTGTGGACGAGTGTGCCCGGCAGGAGGGCTTGACTTTCTTTGGCACAGAGCTAGCC
CCAGTGCGGCAACAGGAGCTGGCCTCTCTGCTAAGAACATACTTGGCACGGCTGCCCTGGGCTGGGCA
GCTGACCAAGGAGCTACCCCTCTCACCTGCTTTCTTTGGTGAGGATGGCATCTTCCGTCATGACCGCC
TCCGCTTCCGGGATTTTGTGGATGCCCTGGAGGACAGCTTGGAGGAGGTGGCTGTGCAACAGACAGGT
GATGATGATGAAGTAGATGACTTTGAGGACTTCATCTTCAGCCACTTCTTTGGAGACAAAGCACTGAA
GAAGAGGCCTCTTCCTCCCCACAGGTCAGGGAAGAAGGACAAGCACTCACAGAGCCCAAGAGCTGCGG
GGCCCAGGGAGGGGCACAGCCATAGCCACCACCACCACCACCGGGGCTGACACCCTGCCCCACAGGGA
ATGGCCTTGGCCTGGCCCAGCCCAAGATCCCAGCGTTATCCAACTCCTGGAGGGTGGACTCTGTCCTG
GCTTGTTTGGTGTCCTCAGATAT

| | | | |
|---|---|---|---|
| NOV16a, CG52171-04 | SEQ ID NO: 434 | 737 aa | MW at 81398.4kD |
| Protein Sequence | | | |

MASCPDSDNSWVLAGSESLPVETLGPASRMDPESEEALQAPHSPSKTDGKELAGTMDGEGTLFQTESP
QSGSILTEETEVKGTLEGDVCGVEPPGPGDTVVQGDLQETTVVTGLGPDTQDLEGQSPPQSLPSTPKA
AWIREEGRCSSSDDDTDVDMEGLRRRRGREAGPPQPMVPLAVENQAGGEGAGGELGISLNMCLLGALV
LLGLGVLLFSGGLSESETGPMEEVERQVLPDPEVLEAVGDRQDGLREQLQAPVPPDSVPSLQNMGLLL
DKLAKENQDIRLLQAQLQAQKEELQSLMHQPKGLEEENAQLRGALQQGEAFQRALESELQQLRARLQG
LEADCVRGPDGVCLSGDRGPQGDKAIREQGPREQEPELSFLKQKEQLEAEAQALRQELERQRRLLGSV
QQDLERSLQDASRGDPAHAGLAELGHRLAQKLQGLENWGQDPGVSANASKAWHQKSHFQNSREWSGKE
KWWDGQRDRKAEHWKHKKEESGRERKKNWGGQEDREPAGRWKEGRPRVEESGSKKEGKRQGPKEPPRK
SGSFHSSGEKQKQPRWREGTKDSHDPLPSWAELLRPKYRAPQGCSGVDECARQEGLTFFGTEIAPVRQ
QELASLLRTYLARLPWAGQLTKELPLSPAFFGEDGIFRHDRLRFRDFVDALEDSLEEVAVQQTGDDDE
VDDFEDFIFSHFFGDKALKKRPLPPHRSGKKDKHSQSPRAAGPREGHSHSHHHHHRG

| | | |
|---|---|---|
| NOV16b, CG52171-01 | SEQ ID NO: 435 | 2739 bp |
| DNA Sequence | ORF Start: ATG at 670 | ORF Stop: TGA at 2275 |

CTGCAACAAGTTAAGCTGAAGACCGAAGCAAGAGCTGGTTCAGGTGGCAGCCACAGCAGCCTCAGGGA
CCTCAGCAACTATGGCCTCCTGCCCAGACTCTGATAATAGCTGGGTGCTTGCTGGCTCCGAGAGCCTC
CCAGTGGAGACACTGGGCCCGGCATCCAGGATGGACCCAGAATCTGAGAGAGCCCTGCAGGCCCCTCA
CAGCCCCTCCAAGACAGATGGGAAAGAATTAGCTGGGACCATGGATGGAGAAGGGACGCGCTTCCAGA
CTGAAAGCCCTCAGTCTGGCAGCATTCTAACAGAGGAGACTGAGGTCAAGGGCACCCTGGAAGGTGAT
GTTTGTGGTGTGGAGCCTCCTGGCCCAGGAGACACAGTAGTCCAGGGAGACCTGCAGGAGACCACCGT
GGTGACAGGCCTGGGACCAGACACACAGGACCTGGAAGGCCAGAGCCCTCCACAGAGCCTGCCTTCAA
CCCCCAAAGCAGCTTGGATCAGGGAGGAGGGCCGCTGCTCCAGCAGTGACGATGACACCGACGTGGAC
ATGGAGGGTCTGCGGAGACGGCGGGGCCGGGAGGCCGGCCCACCTCAGCCCATGGTGCCCCTGGCTGT
GGAGAACCAGGCTGGGGGTGAGGGTGCAGGCGGGGAGCTGGGCATTTTCCCCTCAACATGTGCCTCCT
TGGGGCCCTGGTTCTGCTTGGCCTGGGGGTCCTCCTCTTCTCAGGTGGCCTCTCAGAGTCTGAGACTG
GGCCCATGGAGGAAGTGGAGCGGCAGGTCCTCCCAGACCCCGAGGTGCTGGAAGCTGTGGGGGACAGG
CAGGATGGGCTAAGGGAACAGCTGCAGGCCCCAGTGCCTCCTGACAGTGTCCCCAGCCTGCAAAACAT
GGGTCTTCTGCTGGACAAGCTGGCCAAGGAGAACCAGGACATCCGGCTGCTGCAGGCCCAGCTGCAGG
CCCAAAAGGAAGAGCTTCAGAGCCTGATGCACCAGCCCAAAGGGCTAGAGGAGGAGAATGCCCAGCTC
CGGGGGGCTCTGCAGCAGGGCGAAGCCTTCCAGCGGGCTCTGGAGTCAGAGCTGCAGCAGCTGCGGGC
CCGGCTCCAGGGGCTGGAGGCCGACTGTGTCCGGGGCCCAGATGGGGTGTGCCTCAGTGGGGGTAGAG
GCCCACAGGGTGACAAGGCCATCAGGGAGCAAGGCCCCAGGGAGCAGGAGCCAGAACTCAGCTTCCTG
AAGCAGAAGGAACAGCTGGAGGCTGAGGCACAGGCATTAAGGCAAGAGTTAGAGAGGCAGCGACGGCT
GCTGGGGTCTGTACAGCAGGATCTGGAGAGGAGCTTGCAGGATGCCAGCCGCGGGGACCCAGCTCATG
CTGGCTTGGCTGAGCTGGGCCACAGATTGGCCCAGAAACTGCAGGGCCTGGAGAACTGGGGCCAGGAC
CCTGGGGTCTCTGCCAATGCCTCAAAGGCCTGGCACCAGAAGTCCCACTTCCAGAATTCTAGGGAGTG
GAGTGGAAAGGAAAAGTGGTGGGATGGGCAGAGAGACCGGAAGGCTGAGCACTGGAAACATAAGAAGG
AAGAATCTGGCCGGGAAAGGAAGAAGAACTGGGGAGGTCAGGAGGACAGGGAGCCAGCAGGAAGGTGG
AAGGAGGGCAGGCCAAGGGTGGAGGAGTCGGGGAGCAAGAAGGAGGGCAAGCGACAGGGCCCGAAGGA
ACCCCCAAGGAAAAGTGGTAGCTTCCACTCCTCTGGAGAAAAGCAGAAGCAACCTCGGTGGAGGGAAG
GGACTAAGGGCAGCCATGACCCCCTGCCATCCTGGGCAGAGCTGTTGAGGCCCAAGTACCGGGCACCC
CAGGGCTGCTCAGGTGTGGACGAGTGTGCCCGGCAGGAGGGCCTGACTTTCTTTGGCACAGAGCTAGC
CCCAGTGCGGCAACAGGAGCTGGCCTCTCTGCTAAGAACATACTTGGCACGGCTGCCCTGGGCTGGGC
AGCTGACCAAGGAGCTACCCCTCTCACCTGCTTTCTTTGGTGAGGATGGCATCTTCCGTCATGACCGC
CTCCGCTTCCGGGATTTTGTGGATGCCCTGGAGGACAGCTTGGAGGAGGTGGCTGTGCAACAGACAGG
TGATGATGATGAAGTAGATGACTTTGAGGACTTCATCTTCAGCCACTTCTTTGGAGACAAAGCACTGA
AGAAGAGGTCAGGGAAGAAGGACAAGCACTCACAGAGCCCAAGAGCTGCGGGGCCCAGGGAGGGGCAC
AGCCATAGCCACCACCACCACCACCGGGGCTGACACCCTGCCCCACAGGGAATGGCCTTGGCCTGGCC
CAGCCCAAGATCCCAGCGTTATCTAACTCCTGGAGGGTGGACTCTGTCCTGGCTTGTTTGGTGTCCTC
AGATATCTTTCACACAGTAGAGCAAAATCACCAGCCCTGCACTGATGTCACTTTATGTAGAAAAAGGC
CTTAGCTGGACCTGCGTTGCCGTCTATGCAAATGCATGCAAATACTCCAGGCCCTGGGATGTGGGCTT
GTGTTTTGTCACTGTGAAGGGGGAGATGGGAGAGGAGCCTGTTTTGGGGTGGGGTCTGGGGAAGGCAA
TCTGATTCTGAAGCTAAAGAGCTTTCATCCTCTTGAGTGTATGTCCCCATAGTGGGCCCCTTGACCCA
CATGCTGACCGGTGCCTTGGGATTTGACTAGAGTTGCTGCTTCCAAACCTAACAAAACCCCAGGGTAA
GTCCTCGTGCTGGGCCTCG

| | | | |
|---|---|---|---|
| NOV16b, CG52171-01 | SEQ ID NO: 436 | 535 aa | MW at 60290.7kD |
| Protein Sequence | | | |

MCLLGALVLLGLGVLLFSGGLSESETGPMEEVERQVLPDPEVLEAVGDRQDGLREQLQAPVPPDSVPS
LQNMGLLLDKLAKENQDIRLLQAQLQAQKEELQSLMHQPKGLEEENAQLRGALQQGEAFQRALESELQ
QLRARLQGLEADCVRGPDGVCLSGGRGPQGDKAIREQGPREQEPELSFLKQKEQLEAEAQALRQELER
QRRLLGSVQQDLERSLQDASRGDPAHAGLAELGHRLAQKLQGLENWGQDPGVSANASKAWHQKSHFQN
SREWSGKEKWWDGQRDRKAEHWKHKKEESGRERKKNWGGQEDREPAGRWKEGRPRVEESGSKKEGKRQ
GPKEPPRKSGSFHSSGEKQKQPRWREGTKGSHDPLPSWAELLRPKYRAPQGCSGVDECARQEGLTFFG
TELAPVRQQELASLLRTYLARLPWAGQLTKELPLSPAFFGEDGIFRHDRLRFRDFVDALEDSLEEVAV
QQTGDDDEVDDFEDFIFSHFFGDKALKKRSGKKDKHSQSPRAAGPREGHSHSHHHHHRG

| | | |
|---|---|---|
| NOV16c, CG52171-02 | SEQ ID NO: 437 | 1665 bp |
| DNA Sequence | ORF Start: ATG at 77 | ORF Stop: TGA at 1388 |

CAACAAGTTAAGCTGAAGACCGAAGCAAGAGCTGGTTCAGGTGGCAGCCACAGCAGCCTCAGGGACCT
CAGCAACTATGGCCTCCTGCCCAGACTCTGATAATAGCTGGGTGCTTGCTGGCTCCGAGAGCCTGCCA
GTGGAGACACTGGGCCCGGCATCCAGGATGGACCCAGAATCTGAGAGAGCCCTGCAGGCCCCTCACAG
CCCCTCCAAGACAGATGGGAAAGAATTAGCTGGGACCATGGATGGAGAAGGGACGCTCTTCCAGACTG
AAAGCCCTCAGTCTGGCAGCATTCTAACAGAGGAGACTGAGGTCAAGGGCACCCTGGAAGGTGATGTT
TGTGGTGTGGAGCCTCCTGGCCCAGGAGACACAGTAGTCCAGGGAGACCTGCAGGAGACCACCGTGGT
GACAGGCCTGGGACCAGACACACAGGACCTGGAAGGCCAGAGCCCTCCACAGAGCCTGCCTTCAACCC
CCAAAGCAGCTTGGATCAGGGAGGAGGGCCGCTGCTCCAGCAGTGACGATGACACCGACGTGGACATG
GAGGGTCTGCGGAGACGGCGGGGCCGGGAGGCCGGCCCACCTCAGCCCATGGTGCCCCTGGCTGTGGA
GAACCAGGCTGGGGGTGAGGGTGCAGGCGGGGAGCTGGGCATCTCCCTCAACATGTGCCTCCTTGGGG
CCCTGGGTCTGCTTGGCCTGGGGGTCCTCCTCTTCTCAGGTGGCCTCTCAGAGTCTGAGACTGGGCCC
ATGGAGGAAGTGGAGCGGCAGGTCCTCCCAGACCCCGAGGTGCTGGAAGCTGTGGGGGACAGGCAGGA
TGGGCTAAGGGAACAGCTGCAGGCCCCAGTGCCTCCTGACAGTGTCCCCAGCCTGCAAAACATGGGTC
TTCTGCTGGACAAGCTGGCCAAGGAGAACCAGGACATCCGGCTGCTGCAGGCCCAGCTGCAGGCCCAA
AAGGAAGAGCTTCAGAGCCTGATGCACCAGCCCAAAGGGCTAGAGGAGGAGAATGCCCAGCTCCGGGG
GGCTCTGCAGCAGGGCGAAGCCTTCCAGCGGGCTCTGGAGTCAGAGCTGCAGCAGCTGCGGGCCCGGC
TCCAGGGGCTGGAGGCCGACTGTGTCCGGGGCCCAGATGGGGATGGCATCTTCCGTCATGACCGCCTC
CGCTTCCGGGATTTTGTGGATGCCCTGGAGGACAGCTTGGAGGAGGTGGCTGTGCAACAGACAGGTGA
TGATGATGAAGTAGATGACTTTGAGGACTTCATCTTCAGCCACTTCTTTGGAGACAAAGCACTGAAGA
AGAGGTCAGGGAAGAAGGACAAGCACTCACAGAGCCCAAGAGCTGCGGGGCCCAGGGAGGGGCACAGC
CATAGCCACCACCACCACCACCGGGGCTGACACCCTGCCCCACAGGGAATGGCCTTGGCCTGGCCCAG
CCCAAGATCCCAGCGTTATCTAACTCCTGGAGGGTGGACTCTGTCCTGGCTTGTTTGGTGTCCTCAGA
TATCTTTCACACAGTAGAGCAAAATCACCAGCCCTGCACTGATGTCACTTTATGTAGAAAAAGGCCTT
AGCTGGACCTGCGTTGCCGTCTATGCAAATGCATGCAAATACTCCAGGCCCTGGGATGTGGGCTTGTG
TTTTGTCACTGTGAAGGGGGAGATGGGAGAGGA

| | | | |
|---|---|---|---|
| NOV16c, CG52171-02 | SEQ ID NO: 438 | 437 aa | MW at 47136.5kD |
| Protein Sequence | | | |

MASCPDSDNSWVLAGSESLPVETLGPASRMDPESEHALQAPHSPSKTDGKELAGTMDGEGTLFQTESP
QSGSILTEETEVKGTLEGDVCGVEPPGPGDTVVQGDLQETTVVTGLGPDTQDLEGQSPPQSLPSTPKA
AWIREEGRCSSSDDDTDVDMEGLPRRRGREAGPPQPMVPLAVENQAGGEGAGGELGISLNMCLLGALG
LLGLGVLLFSGGLSESETGPMEEVERQVLPDPEVLEAVGDRQDGLREQLQAPVPPDSVPSLQNMGLLL
DKLAKENQDIRLLQAQLQAQKEELQSLMHQPKGLEEENAQLRGALQQGEAFQRALESELQQLPARLQG
LEADCVRGPDGDGIFRHDRLRFRDFVDALEDSLEEVAVQQTGDDDEVDDFEDFIFSHFFGDKALKKRS
GKKDKHSQSPPAAGPREGHSHSHHHHHRG

| | | |
|---|---|---|
| NOV16d, CG52171-03 | SEQ ID NO: 439 | 2104 bp |
| DNA Sequence | ORF Start: ATG at 76 | ORF Stop: at 2104 |

AACAAGTTAAGCTGAAGACCGAAGCAAGAGCTGGTTCAGGTGGCAGCCACAGCAGCCTCAGGGACCTC
AGCAACTATGGCCTCCTGCCCAGACTCTGATAATAGCTGGGTGCTTGCTGGCTCCGAGAGCCTGCCAG
TGGAGACACTGGGCCCGGCATCCAGGATGGACCCAGAATCTGAGAGAGCCCTGCAGGCCCCTCACAGC
CCCTCCAAGACAGATGGGAAAGAATTAGCTGGGACCATGGATGGAGAAGGGACGCTCTTCCAGACTGA
AAGCCCTCAGTCTGGCAGCATTCTAACAGAGGAGACTGAGGTCAAGGGCACCCTGGAAGGTGATGTTT
GTGGTGTGGAGCCTCCTGGCCCAGGAGACACAGTAGTCCAGGGAGACCTGCAGGAGACCACCGTGGTG
ACAGGCCTGGGACCAGACACACAGGACCTGGAAGGCCAGAGCCCTCCACAGAGCCTGCCTTCAACCCC
CAAAGCAGCTTGGATCAGGGAGGAGGGCCGCTGCTCCAGCAGTGACGATGACACCGACGTGGACATGG
AGGGTCTGCGGAGACGGCGGGGCCGGGAGGCCGGCCCACCTCAGCCCATGGTGCCCCTGGCTGTGGAG
AACCAGGCTGGGGGTGAGGGTGCAGGCGGGGAGCTGGGCATCTCCCTCAACATGTGCCTCCTTGGGGC
CCTGGGTCTGCTTGGCCTGGGGGTCCTCCTCTTCTCAGGTGGCCTCTCAGAGTCTGAGACTGGGCCCA
TGGAGGAAGTGGAGCGGCAGGTCCTCCCAGACCCCGAGGTGCTGGAAGCTGTGGGGGACAGGCAGGAT
GGGCTAAGGGAACAGCTGCAGGCCCCAGTGCCTCCTGACAGTGTCCCCAGCCTGCAAAACATGGGTCT
TCTGCTGGACAAGCTGGCCAAGGAGAACCAGGACATCCGGCTGCTGCAGGCCCAGCTGCAGGCCCAAA
AGGAAGAGCTTCAGAGCCTGATGCACCAGCCCAAAGGGCTAGAGGAGGAGAATGCCCAGCTCCGGGGG
GCTCTGCAGCAGGGCGAAGCCTTCCAGCGGGCTCTGGAGTCAGAGCTGCAGCAGCTGCGGGCCCGGCT
CCAGGGGCTGGAGGCCGACTGTGTCCGGGGCCCAGATGGGGTGTGCCTCAGTGGGGGTAGAGGCCCAC
AGGGTGACAAGGCCATCAGGGAGCAAGGCCCCAGGGAGCAGGAGCCAGAACTCAGCTTCCTGAAGCAG
AAGGAACAGCTGGAGGCTGAGGCACAGGCATTAAGGCAAGAGTTAGAGAGGCAGCGACGGCTGCTGGG
GTCTGTACAGCAGGATCTGGAGAGGAGCTTGCAGGATGCCAGCCGCGGGGACCCAGCTCATGCTGGCT
TGGCTGAGCTGGGCCACAGATTGGCCCAGAAACTGCAGGGCCTGGAGAACTGGGGCCAGGACCCTGGG
GTCTCTGCCAATGCCTCAAAGGCCTGGCACCAGAAGTCCCACTTCCAGAATTCTAGGGAGTGGAGTGG
AAAGGAAAAGTGGTGGGATGGGCAGAGAGACCGGAAGGCTGAGCACTGGAAACATAAGAAGGAAGAAT
CTGGCCGGGAAAGGAAGAAGAACTGGGGAGGTCAGGAGGACAGGGAGCCAGCAGGAAGGTGGAAGGAG
GGCAGGCCAAGGGTGGAGGAGTCGGGGAGCAAGAAGGAGGGCAAGCGACAGGGCCCGAAGGAACCCCC
AAGGAAAAGTGGTAGCTTCCACTCCTCTGGAGAAAAGCAGAAGCAACCTCGGTGGAGGGAAGGGACTA
AGGGCAGCCATGACCCCCTGCCATCCTGGGCAGAGCTGTTGAGGCCCAAGTACCGGGCACCCCAGGGC
TGCTCAGGTGTGGACGAGTGTGCCCGGCAGGAGGGCCTGACTTTCTTTGGCACAGAGCTAGCCCCAGT
GCGGCAACAGGAGCTGGCCTCTCTGCTAAGAACATACTTGGCACGGCTGCCCTGGGCTGGGCAGCTGA
CCAAGGAGCTACCCCTCTCACCTGCTTTCTTTGGTGAGGATGGCATCTTCCGTCATGACCGCCTCCGC
TTCCGGGATTTTGTGGATGCCCTGGAGGACAGCTTGGAGGAGGTTCTAGAATTCAGCGGCCGCT

| | | | |
|---|---|---|---|
| NOV16d, CG52171-03 | SEQ ID NO: 440 | 676 aa | MW at 74306.8kD |
| Protein Sequence | | | |

MASCPDSDNSWVLAGSESLPVETLGPASRMDPESERALQAPHSPSKTDGKELAGTMDGEGTLFQTESP
QSGSILTEETEVKGTLEGDVCGVEPPGPGDTVVQGDLQETTVVTGLGPDTQDLEGQSPPQSLPSTPKA
AWIREEGRCSSSDDDTDVDMEGLRRRRGREAGPPQPMVPLAVENQAGGEGAGGELGISLNMCLLGALG
LLGLGVLLFSGGLSESETGPMEEVERQVLPDPEVLEAVGDRQDGLREQLQAPVPPDSVPSLQNMGLLL
DKLAKENQDIRLLQAQLQAQKEELQSLMHQPKGLEEENAQLRGALQQGEAFQRALESELQQLRARLQG
LEADCVRGPDGVCLSGGRGPQGDKAIREQGPREQEPELSFLKQKEQLEAEAQALRQELERQRRLLGSV
QQDLERSLQDASRGDPAHAGLAELGHRLAQKLQGLENWGQDPGVSANASKAWHQKSHFQNSREWSGKE
KWWDGQRDRKAEHWKHKKEESGRERKKNWGGQEDREPAGRWKEGRPRVEESGSKKEGKRQGPKEPPRK
SGSFHSSGEKQKQPRWREGTKGSHDPLPSWAELLRPKYRAPQGCSGVDECARQEGLTFFGTELAPVRQ
QELASLLRTYLARLPWAGQLTKELPLSPAFFGEDGIFRHDRLRFRDFVDALEDSLEEVLEFSGR

| | | |
|---|---|---|
| NOV16e, CG52171-05 | SEQ ID NO: 441 | 1539 bp |
| DNA Sequence | ORF Start: at 1 | ORF Stop: end of sequence |

GAGTCTGAGACTGGGCCCATGGAGGAAGTGGAGCGGCAGGTCCTCCCAGACCCCGAGGTGCTGGAAGC
TGTGGGGGACAGGCAGGATGGGCTAAGGGAACAGCTGCAGGCCCCAGTGCCTCCTGACAGTGTCCCCA
GCCTGCAAAACATGGGTCTTCTGCTGGACAAGCTGGCCAAGGAGAACCAGGACATCCGGCTGCTGCAG
GCCCAGCTGCAGGCCCAAAAGGAAGAGCTTCAGAGCCTGATGCACCAGCCCAAAGGGCTAGAGGAGGA
GAATGCCCAGCTCCGGGGGGCTCTGCAGCAGGGCGAAGCCTTCCAGCGGGCTCTGGAGTCAGAGCTGC
AGCAGCTGCGGGCCCGGCTCCAGGGGCTGGAGGCCGACTGTGTCCGGGGCCCAGATGGGGTGTGCCTC
AGTGGGGATAGAGGCCCACAGGGTGACAAGGCCATCAGGGAGCAAGGCCCCAGGGAGCAGGAGCCAGA
ACTCAGCTTCCTGAAGCAGAAGGAACAGCTGGAGGCTGAGGCACAGGCATTAAGGCAAGAGTTAGAGA
GGCAGCGACGGCTGCTGGGGTCTGTACAGCAGGATCTGGAGAGGAGCTTGCAGGATGCCAGCCGCGGG
GACCCAGCTCATGCTGGCTTGGCTGAGCTGGGCCACAGATTGGCCCAGAAACTGCAGGGCCTGGAGAA
CTGGGGCCAGGACCCTGGGGTCTCTGCCAATGCCTCAAAGGCCTGGCACCAGAAGTCCCACTTCCAGA
ATTCTAGGGAGTGGAGTGGAAAGGAAAAGTGGTGGGATGGGCAGAGAGACCGGAAGGCTGAGCACTGG
AAACATAAGAAGGAAGAATCTGGCCGGGAAAGGAAGAAGAACTGGGGAGGTCAGGAGGACAGGGAGCC
AGCAGGAAGGTGGAAGGAGGGCAGGCCAAGGGTGGAGGAGTCGGGGAGCAAGAAGGAGGGCAAGCGAC
AGGGCCCGAAGGAACCCCCAAGGAAAAGTGGTAGCTTCCACTCCTCTGGAGAAAAGCAGAAGCAACCT
CGGTGGAGGGAAGGGACTAAGGACAGCCATGACCCCCTGCCATCCTGGGCAGAGCTGTTGAGGCCCAA
GTACCGGGCACCCCAGGGCTGCTCAGGTGTGGACGAGTGTGCCCGGCAGGAGGGCCTGACTTTCTTTG
GCACAGAGCTAGCCCCAGTGCGGCAACAGGAGCTGGCCTCTCTGCTAAGAACATACTTGGCACGGCTG
CCCTGGGCTGGGCAGCTGACCAAGGAGCTACCCCTCTCACCTGCTTTCTTTGGTGAGGATGGCATCTT
CCGTCATGACCGCCTCCGCTTCCGGGATTTTGTGGATGCCCTGGAGGACAGCTTGGAGGAGGTGGCTG
TGCAACAGACAGGTGATGATGATGAAGTAGATGACTTTGAGGACTTCATCTTCAGCCACTTCTTTGGA
GACAAAGCACTGAAGAAGAGGTCAGGGAAGAAGGACAAGCACTCACAGAGCCCAAGAGCTGCGGGGCC
CAGGGAGGGGCACAGCCATAGCCACCACCACCACCACCGGGGC

| | | | |
|---|---|---|---|
| NOV16e, CG52171-05 | SEQ ID NO: 442 | 513 aa | MW at 58278.1kD |
| Protein Sequence | | | |

ESETGPMEEVERQVLPDPEVLEAVGDRQDGLREQLQAPVPPDSVPSLQNMGLLLDKLAKENQDIRLLQ
AQLQAQKEELQSLMHQPKGLEEENAQLRGALQQGEAFQRALESELQQLRARLQGLEADCVRGPDGVCL
SGDRGPQGDKAIREQGPREQEPELSFLKQKEQLEAEAQALRQELERQRRLLGSVQQDLERSLQDASRG
DPAHAGLAELGHRLAQKLQGLENWGQDPGVSANASKAWHQKSHFQNSREWSGKEKWWDGQRDRKAEHW
KHKKEESGRERKKNWGGQEDREPAGRWKEGRPRVEESGSKKEGKRQGPKEPPRKSGSFHSSGEKQKQP
RWREGTKDSHDPLPSWAELLRPKYPAPQGCSGVDECARQEGLTFFGTELAPVRQQELASLLRTYLARL
PWAGQLTKELPLSPAFFGEDGIFRHDRLRFRDFVDALEDSLEEVAVQQTGDDDEVDDFEDFIFSHFFG
DKALKKRSGKKDKHSQSPPAAGPREGHSHSHHHHHRG

| | | |
|---|---|---|
| NOV16f, CG52171-06 | SEQ ID NO: 443 | 2366 bp |
| DNA Sequence | ORF Start: ATG at 76 | ORF Stop: TGA at 2266 |

GAGAAGTTAAGCTGAAGACCGAAGCAAGAGCTGGTTCAGGTGGCAGCCACAGCAGCCTCAGGGACCTC
AGCAACTATGGCCTCCTGCCCAGACTCTGATAATAGCTGGGTGCTTGCTGGCTCCGAGAGCCTGCCAG
TGGAGACACTGGGCCCGGCATCCAGGATGGACCCAGAATCTGAGAGAGCCCTGCAGGCCCCTCACAGC
CCCTCCAAGACAGATGGGAAAGAATTAGCTGGGACCATGGATGGAGAAGGGACGCTCTTCCAGACTGA
AAGCCCTCAGTCTGGCAGCATTCTAACAGAGGAGACTGAGGTCAAGGGCACCCTGGAAGGTGATGTTT
GTGGTGTGGAGCCTCCTGGCCCAGGAGACACAGTAGTCCAGGGAGACCTGCAGGAGACCACCGTGGTG
ACAGGCCTGGGACCAGACACACAGGACCTGGAAGGCCAGAGCCCTCCACAGAGCCTGCCTTCAACCCC
CAAAGCAGCTTGGATCAGGGAGGAGGGCCGCTGCTCCAGCAGTGACGATGACACCGACGTGGACATGG
AGGGTCTGCGGAGACGGCGGGGCCGGGAGGCCGGCCCACCTCAGCCCATGGTGCCCCTGGCTGTGGAG
AACCAGGCTGGGGGTGAGGGTGCAGGCGGGGAGCTGGGCATCTCCCTCAACATGTGCCTCCTTGGGGC
CCTGGTTCTGCTTGGCCTGGGGGTCCTCCTCTTCTCAGGTGGCCTCTCAGAGTCTGAGACTGGGCCCA
TGGAGGAAGTGGAGCGGCAGGTCCTCCCAGACCCCGAGGTGCTGGAAGCTGTGGGGGACAGGCAGGAT
GGGCTAAGGGAACAGCTGCAGGCCCCAGTGCCTCCTGACAGTGTCCCCAGCCTGCAAAACATGGGTCT
TCTGCTGGACAAGCTGGCCAAGGAGAACCAGGACATCCGGCTGCTGCAGGCCCAGCTGCAGGCCCAAA
AGGAAGAGCTTCAGAGCCTGATGCACCAGCCCAAAGGGCTAGAGGAGGAGAATGCCCAGCTCCGGGGG
GCTCTGCAGCAGGGCGAAGCCTTCCAGCGGGCTCTGGAGTCAGAGCTGCAGCAGCTGCGGGCCCGGCT
CCAGGGGCTGGAGGCCGACTGTGTCCGGGGCCCAGATGGGGTGTGCCTCAGTGGGGATAGAGGCCCAC
AGGGTGACAAGGCCATCAGGGAGCAAGGCCCCAGGGAGCAGGAGCCAGAACTCAGCTTCCTGAATCAG
AAGGAACAGCTGGAGGCTGAGGCACAGGCATTAAGGCAAGAGTTAGAGAGGCAGCGACGGCTGCTGGG
GTCTGTACAGCAGGATCTGGAGAGGAGCTTGCAGGATGCCAGCCGCGGGGACCCAGCTCATGCTGGCT
TGGCTGAGCTGGGCCACAGATTGGCCCAGAAACTGCAGGGCCTGGAGAACTGGGGCCAGGACCCTGGG
GTCTCTGCCAATGCCTCAAAGGCCTGGCACCAGAAGTCCCACTTCCAGAATTCTAGGGAGTGGAGTGG
AAAGGAAAAGTGGTGGGATGGGCAGAGAGACCGGAAGGCTGAGCACTGGAAACATAAGAAGGAAGAAT
CTGGCCGGGAAAGGAAGAAGAACTGGGGAGGTCAGGAGGACAGGGAGCCAGCAGGAAGGTGGAAGGAG
GGCAGGCCAAGGGTGGAGGAGTCGGGGAGCAAGAAGGAGGGCAAGCGACAGGCCCCGAAGGAACCCCC
AAGGAAAAGTGGTAGCTTCCACTCCTCTGGAGAAAAGCAGAAGCAACCTCGGTGGAGGGAAGGGACTA
AGGACAGCCATGACCCCCTGCCATCCTGGGCAGAGCTGTTGAGGCCCAAGTACCGGGCACCCCAGGGC
TGCTCAGGTGTGGACGAGTGTGCCCGGCAGGAGGGCCTGACTTTCTTTGGCACAGAGCTAGCCCCAGT
GCGGCAACAGGAGCTGGCCTCTCTGCTAAGAACATACTTGGCACGGCTGCCCTGGGCTGGGCAGCTGA
CCAAGGAGCTACCCCTCTCACCTGCTTTCTTTGGTGAGGATGGCATCTTCCGTCATGACCGCCTCCGC
TTCCGGGATTTTGTGGATGCCCTGGAGGACAGCTTGGAGGAGGTGGCTGTGCAACAGACAGGTGATGA
TGATGAAGTAGATGACTTTGAGGACTTCATCTTCAGCCACTTCTTTGGAGACAAAGCACTGAAGAAGA
GGTCAGGGAAGAAGGACAAGCACTCACAGAGCCCAAGAGCTGCGGGGCCCAGGGAGGGGCACAGCCAT
AGCCACCACCACCACCGGGGCTGACACCCTGCCCCACAGGGAATGGCCTTGGCCTGGCCCAGCCCAAG
ATCCCAGCGTTATCTAACTCCTGGAGGGTGGACTCTGTCCTGGCTTGTTTGGTG

| | | | |
|---|---|---|---|
| NOV16f, CG52171-06 | SEQ ID NO: 444 | 730 aa | MW at 80563.4kD |
| Protein Sequence | | | |

MASCPDSDNSWVLAGSESLPVETLGPASRMDPESERALQAPHSPSKTDGKELAGTMDGEGTLFQTESP
QSGSILTEETEVKGTLEGDVCGVEPPGPGDTVVQGDLQETTVVTGLGPDTQDLEGQSPPQSLPSTPKA
AWIREEGRCSSSDDDTDVDMEGLRRRRGREAGPPQPMVPLAVENQAGGEGAGGELGISLNMCLLGALV
LLGLGVLLFSGGLSESETGPMEEVERQVLPDPEVLEAVGDRQDGLREQLQAPVPPDSVPSLQNMGLLL
DKLAKENQDIRLLQAQLQAQKEELQSLMHQPKGLEEENAQLRGALQQGEAFQRALESELQQLRARLQG
LEADCVRGPDGVCLSGDRGPQGDKAIREQGPREQEPELSFLNQKEQLEAEAQALRQELERQRRLLGSV
QQDLERSLQDASRGDPAHAGLAELGHRLAQKLQGLENWGQDPGVSANASKAWHQKSHFQNSREWSGKE
KWWDGQRDRKAEHWKHKKEESGRERKKNWGGQEDREPAGRWKEGRPRVEESGSKKEGKRQAPKEPPRK
SGSFHSSGEKQKQPRWREGTKDSHDPLPSWAELLRPKYRAPQGCSGVDECARQEGLTFFGTELAPVRQ
QELASLLRTYLARLPWAGQLTKELPLSPAFFGEDGIFRHDRLRFRDFVDALEDSLEEVAVQQTGDDDE
VDDFEDFIFSHFFGDKALKKRSGKKDKHSQSPRAAGPREGHSHSHHHHRG

| | | |
|---|---|---|
| NOV16g, 5NP13374628 | SEQ ID NO: 445 | 2403 bp |
| of CG52171-04, | ORF Start: ATG at 81 | ORF Stop: TGA at 2292 |
| DNA Sequence | SNP Pos: 373 | SNP Change: A to G |

GGCACGAGAAGTTAAGCTGAAGACCGAAGCAAGAGCTGGTTCAGGTGGCAGCCACAGCAGCCTCAGGG
ACCTCAGCAACTATGGCCTCCTGCCCAGACTCTGATAATAGCTGGGTGCTTGCTGGCTCCGAGAGCCT
GCCAGTGGAGACACTGGGCCCGGCATCCAGGATGGACCCAGAATCTGAGAGAGCCCTGCAGGCCCCTC
ACAGCCCCTCCAAGACAGATGGGAAAGAATTAGCTGGGACCATGGATGGAGAAGGGACGCTCTTCCAG
ACTGAAAGCCCTCAGTCTGGCAGCATTCTAACAGAGGAGACTGAGGTCAAGGGCACCCTGGAAGGTGA
TGTTTGTGGTGTGGAGCCTCCTGGCCCAGGAGGCACAGTAGTCCAGGGAGACCTGCAGGAGACCACCG
TGGTGACAGGCCTGGGACCAGACACACAGGACCTGGAAGGCCAGAGCCCTCCACAGAGCCTGCCTTCA
ACCCCCAAAGCAGCTTGGATCAGGGAGGAGGGCCGCTGCTCCAGCAGTGACGATGACACCGACGTGGA
CATGGAGGGTCTGCGGAGACGGCGGGGCCGGGAGGCCGGCCCACCTCAGCCCATGGTGCCCCTGGCTG
TGGAGAACCAGGCTGGGGGTGAGGGTGCAGGCGGGGAGCTGGGCATCTCCCTCAACATGTGCCTCCTT
GGGGCCCTGGTTCTGCTTGGCCTGGGGGTCCTCCTCTTCTCAGGTGGCCTCTCAGAGTCTGAGACTGG
GCCCATGGAGGAAGTGGAGCGGCAGGTCCTCCCAGACCCCGAGGTGCTGGAAGCTGTGGGGGACAGGC
AGGATGGGCTAAGGGAACAGCTGCAGGCCCCAGTGCCTCCTGACAGTGTCCCCAGCCTGCAAAACATG
GGTCTTCTGCTGGACAAGCTGGCCAAGGAGAACCAGGACATCCGGCTGCTGCAGGCCCAGCTGCAGGC
CCAAAAGGAAGAGCTTCAGAGCCTGATGCACCAGCCCAAAGGGCTAGAGGAGGAGAATGCCCAGCTCC
GGGGGGCTCTGCAGCAGGGCGAAGCCTTCCAGCGGGCTCTGGAGTCAGAGCTGCAGCAGCTGCGGGCC
CGGCTCCAGGGGCTGGAGGCCGACTGTGTCCGGGGCCCAGATGGGGTGTGCCTCAGTGGGGATAGAGG
CCCACAGGGTGACAAGGCCATCAGGGAGCAAGGCCCCAGGGAGCAGGAGCCAGAACTCAGCTTCCTGA
AGCAGAAGGAACAGCTGGAGGCTGAGGCACAGGCATTAAGGCAAGAGTTAGAGAGGCAGCGACGGCTG
CTGGGGTCTGTACAGCAGGATCTGGAGAGGAGCTTGCAGGATGCCAGCCGCGGGGACCCAGCTCATGC
TGGCTTGGCTGAGCTGGGCCACAGATTGGCCCAGAAACTGCAGGGCCTGGAGAACTGGGGCCAGGACC
CTGGGGTCTCTGCCAATGCCTCAAAGGCCTGGCACCAGAAGTCCCACTTCCAGAATTCTAGGGAGTGG
AGTGGAAAGGAAAAGTGGTGGGATGGGCAGAGAGACCGGAAGGCTGAGCACTGGAAACATAAGAAGGA
AGAATCTGGCCGGGAAAGGAAGAAGAACTGGGGAGGTCAGGAGGACAGGGAGCCAGCAGGAAGGTGGA
AGGAGGGCAGGCCAAGGGTGGAGGAGTCGGGGAGCAAGAAGGAGGGCAAGCGACAGGGCCCGAAGGAA
CCCCCAAGGAAAAGTGGTAGCTTCCACTCCTCTGGAGAAAAGCAGAAGCAACCTCGGTGGAGGGAAGG
GACTAAGGACAGCCATGACCCCCTGCCATCCTGGGCAGAGCTGTTGAGGCCCAAGTACCGGGCACCCC
AGGGCTGCTCAGGTGTGGACGAGTGTGCCCGGCAGGAGGGCTTGACTTTCTTTGGCACAGAGCTAGCC
CCAGTGCGGCAACAGGAGCTGGCCTCTCTGCTAAGAACATACTTGGCACGGCTGCCCTGGGCTGGGCA
GCTGACCAAGGAGCTACCCCTCTCACCTGCTTTCTTTGGTGAGGATGGCATCTTCCGTCATGACCGCC
TCCGCTTCCGGGATTTTGTGGATGCCCTGGAGGACAGCTTGGAGGAGGTGGCTGTGCAACAGACAGGT
GATGATGATGAAGTAGATGACTTTGAGGACTTCATCTTCAGCCACTTCTTTGGAGACAAAGCACTGAA
GAAGAGGCCTCTTCCTCCCCACAGGTCAGGGAAGAAGGACAAGCACTCACAGAGCCCAAGAGCTGCGG
GGCCCAGGGAGGGGCACAGCCATAGCCACCACCACCACCACCGGGGCTGACACCCTGCCCCACAGGGA
ATGGCCTTGGCCTGGCCCAGCCCAAGATCCCAGCGTTATCCAACTCCTGGAGGGTGGACTCTGTCCTG
GCTTGTTTGGTGTCCTCAGATAT

| | | | |
|---|---|---|---|
| NOV16g, SNP13374628 | SEQ ID NO: 446 | 737 aa | MW at 81340.4kD |
| of CG52171-04, | SNP Pos: 98 | | SNP Change: |
| Protein Sequence | | | Asp to Gly |

MASCPDSDNSWVLAGSESLPVETLGPASRMDPESERALQAPHSPSKTDGKELAGTMDGEGTLFQTESP
QSGSILTEETEVKGTLEGDVCGVEPPGPGGTVVQGDLQETTVVTGLGPDTQDLEGQSPPQSLPSTPKA
AWIREEGRCSSSDDDTDVDMEGLRRRRGREAGPPQPMVPLAVENQAGGEGAGGELGISLNMCLLGALV
LLGLGVLLFSGGLSESETGPMEEVERQVLPDPEVLEAVGDRQDGLREQLQAPVPPDSVPSLQNMGLLL
DKLAKENQDIRLLQAQLQAQKEELQSLMHQPKGLEEENAQLRGALQQGEAFQRALEsELQQLRARLQG
LEADCVRGPDGVCLSGDRGPQGDKAIREQGPREQEPELSFLKQKEQLEAEAQALRQELERQRPLLGSV
QQDLERSLQDASRGDPAHAGLAELGHPLAQKLQGLENWGQDPGVSANASKAWHQKSHFQNSREWSGKE
KWWDGQRDRKAEHWKHKKEESGRERKKNWGGQEDREPAGRWKEGRPRVEESGSKKEGKRQGPKEPPRK
SGSFHSSGEKQKQPRWREGTKDSHDPLPSWAELLRPKYRAPQGCSGVDECARQEGLTFFGTELAPVRQ
QELASLLRTYLARLPWAGQLTKELPLSPAFFGEDGIFRHDRLRFRDFVDALEDSLEEVAVQQTGDDDE
VDDFEDFIFSHFFGDKALKKRPLPPHRSGKKDKHSQSPRAAGPREGHSHSHHHHHRG

| | | |
|---|---|---|
| NOV16h, SNP13377329 | SEQ ID NO: 447 | 2403 bp |
| of CG52171-04, | ORF Start: ATG at 81 | ORF Stop: TGA at 2292 |
| DNA Sequence | SNP Pos: 1150 | SNP Change: A to G |

GGCACGAGAAGTTAAGCTGAAGACCGAAGCAAGAGCTGGTTCAGGTGOCAGCCACAGCAGCCTCAGGG
ACCTCAGCAACTATGGCCTCCTGCCCAGACTCTGATAATAGCTGGGTGCTTGCTGGCTCCGAGAGCCT
GCCAGTGGAGACACTGGGCCCGGCATCCAGGATGGACCCAGAATCTGAGAGAGCCCTGCAGGCCCCTC
ACAGCCCCTCCAAGACAGATGGGAAAGAATTAGCTGGGACCATGGATGGAGAAGGGACGCTCTTCCAG
ACTGAAAGCCCTCAGTCTGGCAGCATTCTAACAGAGGAGACTGAGGTCAAGGGCACCCTGGAAGGTGA
TGTTTGTGGTGTGGAGCCTCCTGGCCCAGGAGACACAGTAGTCCAGGGAGACCTGCAGGAGACCACCG
TGGTGACAGGCCTGGGACCAGACACACAGGACCTGGAAGGCCAGAGCCCTCCACAGAGCCTGCCTTCA
ACCCCCAAAGCAGCTTGGATCAGGGAGGAGGGCCGCTGCTCCAGCAGTGACGATGACACCGACGTGGA
CATGGAGGGTCTGCGGAGACGGCGGGGCCGGGAGGCCGGCCCACCTCAGCCCATGGTGCCCCTGGCTG
TGGAGAACCAGGCTGGGGGTGAGGGTGCAGGCGGGGAGCTGGGCATCTCCCTCAACATGTGCCTCCTT
GGGGCCCTGGTTCTGCTTGGCCTGGGGGTCCTCCTCTTCTCAGGTGGCCTCTCAGAGTCTGAGACTGG
GCCCATGGAGGAAGTGGAGCGGCAGGTCCTCCCAGACCCCGAGGTGCTGGAAGCTGTGGGGGACAGGC
AGGATGGGCTAAGGGAACAGCTGCAGGCCCCAGTGCCTCCTGACAGTGTCCCCAGCCTGCAAAACATG
GGTCTTCTGCTGGACAAGCTGGCCAAGGAGAACCAGGACATCCGGCTGCTGCAGGCCCAGCTGCAGGC
CCAAAAGGAAGAGCTTCAGAGCCTGATGCACCAGCCCAAAGGGCTAGAGGAGGAGAATGCCCAGCTCC
GGGGGGCTCTGCAGCAGGGCGAAGCCTTCCAGCGGGCTCTGGAGTCAGAGCTGCAGCAGCTGCGGGCC
CGGCTCCAGGGGCTGGAGGCCGACTGTGTCCGGGGCCCAGATGGGGTGTGCCTCAGTGGGGGTAGAGG
CCCACAGGGTGACAAGGCCATCAGGGAGCAAGGCCCCAGGGAGCAGGAGCCAGAACTCAGCTTCCTGA
AGCAGAAGGAACAGCTGGAGGCTGAGGCACAGGCATTAAGGCAAGAGTTAGAGAGGCAGCGACGGCTG
CTGGGGTCTGTACAGCAGGATCTGGAGAGGAGCTTGCAGGATGCCAGCCGCGGGGACCCAGCTCATGC
TGGCTTGGCTGAGCTGGGCCACAGATTGGCCCAGAAACTGCAGGGCCTGGAGAACTGGGGCCAGGACC
CTGGGGTCTCTGCCAATGCCTCAAAGGCCTGGCACCAGAAGTCCCACTTCCAGAATTCTAGGGAGTGG
AGTGGAAAGGAAAAGTGGTGGGATGGGCAGAGAGACCGGAAGGCTGAGCACTGGAAACATAAGAAGGA
AGAATCTGGCCGGGAAAGGAAGAAGAACTGGGGAGGTCAGGAGGACAGGGAGCCAGCAGGAAGGTGGA
AGGAGGGCAGGCCAAGGGTGGAGGAGTCGGGGAGCAAGAAGGAGGGCAAGCGACAGGGCCCGAAGGAA
CCCCCAAGGAAAAGTGGTAGCTTCCACTCCTCTGGAGAAAAGCAGAAGCAACCTCGGTGGAGGGAAGG
GACTAAGGACAGCCATGACCCCCTGCCATCCTGGGCAGAGCTGTTGAGGCCCAAGTACCGGGCACCCC
AGGGCTGCTCAGGTGTGGACGAGTGTGCCCGGCAGGAGGGCTTGACTTTCTTTGGCACAGAGCTAGCC
CCAGTGCGGCAACAGGAGCTGGCCTCTCTGCTAAGAACATACTTGGCACGGCTGCCCTGGGCTGGGCA
GCTGACCAAGGAGCTACCCCTCTCACCTGCTTTCTTTGGTGAGGATGGCATCTTCCGTCATGACCGCC
TCCGCTTCCGGGATTTTGTGGATGCCCTGGAGGACAGCTTGGAGGAGGTGGCTGTGCAACAGACAGGT
GATGATGATGAAGTAGATGACTTTGAGGACTTCATCTTCAGCCACTTCTTTGGAGACAAAGCACTGAA
GAAGAGGCCTCTTCCTCCCCACAGGTCAGGGAAGAAGGACAAGCACTCACAGAGCCCAAGAGCTGCGG
GGCCCAGGGAGGGGCACAGCCATAGCCACCACCACCACCACCGGGGCTGACACCCTGCCCCACAGGGA
ATGGCCTTGGCCTGGCCCAGCCCAAGATCCCAGCGTTATCCAACTCCTGGAGGGTGGACTCTGTCCTG
GCTTGTTTGGTGTCCTCAGATAT

| | | | |
|---|---|---|---|
| NOV16h, SNP13377329 | SEQ ID NO: 448 | 737 aa | MW at 81340.4kD |
| of CG52171-04, | SNP Pos: 357 | | SNP Change: |
| Protein Sequence | | | Asp to Gly |

MASCPDSDNSWVLAGSESLPVETLGPASRMDPESERALQAPHSPSKTDGKELAGTMDGEGTLFQTESP
QSGSILTEETEVKGTLEGDVCGVEPPGPGDTVVQGDLQETTVVTGLGPDTQDLEGQSPPQSLPSTPKA
AWIREEGRCSSSDDDTDVDMEGLRRRRGREAGPPQPMVPLAVENQAGGEGAGGELGISLNMCLLGALV
LLGLGVLLFSGGLSESETGPMEEVERQVLPDPEVLEAVGDRQDGLREQLQAPVPPDSVPSLQNMGLLL
DKLAKENQDIRLLQAQLQAQKEELQSLMHQPKGLEEENAQLRGALQQGEAFQRALESELQQLRARLQG
LEADCVRGPDGVCLSGGRGPQGDKAIRBQGPREQEPELSFLKQKEQLEAEAQALRQELERQRRLLGSV
QQDLERSLQDASRGDPAHAGLAELGHRLAQKLQGLENWGQDPGVSANASKAWHQKSHFQNSREWSGKE
KWWDGQRDRKAEHWKHKKEESGRERKKNWGGQEDREPAGRWKEGRPRVEESGSKKEGKRQGPKEPPRK
SGSFHSSGEKQKQPRWREGTKDSHDPLPSWAELLRPKYRAPQGCSGVDECARQEGLTFFGTELAPVRQ
QELASLLRTYLARLPWAGQLTKELPLSPAFFGEDGIFRHDRLRFRDFVDALEDSLEEVAVQQTGDDDE
VDDFEDFIFSHFFGDKALKKRPLPPHRSGKKDKHSQSPRAAGPREGHSHSHHHHHRG

| | | |
|---|---|---|
| NOV16i, SNP13374627 | SEQ ID NO: 449 | 2403 bp |
| of CG52171-04, | ORF Start: ATG at 81 | ORF Stop: TGA at 2292 |
| DNA Sequence | SNP Pos: 1903 | SNP Change: C to T |

GGCACGAGAAGTTAAGCTGAAGACCGAAGCAAGAGCTGGTTCAGGTGGCAGCCACAGCAGCCTCAGGG
ACCTCAGCAACTATGGCCTCCTGCCCAGACTCTGATAATAGCTGGGTGCTTQCTGGCTCCGAGAGCCT
GCCAGTGGAGACACTGGGCCCGGCATCCAGGATGGACCCAGAATCTGAGAGAGCCCTGCAGGCCCCTC
ACAGCCCCTCCAAGACAGATGGGAAAGAATTAGCTGGGACCATGGATGGAGAAGGGACGCTCTTCCAG
ACTGAAAGCCCTCAGTCTGGCAGCATTCTAACAGAGGAGACTGAGGTCAAGGGCACCCTGGAAGGTGA
TGTTTGTGGTGTGGAGCCTCCTGGCCCAGGAGACACAGTAGTCCAGGGAGACCTGCAGGAGACCACCG
TGGTGACAGGCCTGGGACCAGACACACAGGACCTGGAAGGCCAGAGCCCTCCACAGAGCCTGCCTTCA
ACCCCCAAAGCAGCTTGGATCAGGGAGGAGGGCCGCTGCTCCAGCAGTGACGATGACACCGACGTGGA
CATGGAGGGTCTGCGGAGACGGCGGGGCCGGGAGGCCGGCCCACCTCAGCCCATGGTGCCCCTGGCTG
TGGAGAACCAGGCTGGGGGTGAGGGTGCAGGCGGGGAGCTGGGCATCTCCCTCAACATGTGCCTCCTT
GGGGCCCTGGTTCTGCTTGGCCTGGGGGTCCTCCTCTTCTCAGGTGGCCTCTCAGAGTCTGAGACTGG
GCCCATGGAGGAAGTGGAGCGGCAGGTCCTCCCAGACCCCGAGGTGCTGGAAGCTGTGGGGGACAGGC
AGGATGGGCTAAGGGAACAGCTGCAGGCCCCAGTGCCTCCTGACAGTGTCCCCAGCCTGCAAAACATG
GGTCTTCTGCTGGACAAGCTGGCCAAGGAGAACCAGGACATCCGGCTGCTGCAGGCCCAGCTGCAGGC
CCAAAAGGAAGAGCTTCAGAGCCTGATGCACCAGCCCAAAGGGCTAGAGGAGGAGAATGCCCAGCTCC
GGGGGGCTCTGCAGCAGGGCGAAGCCTTCCAGCGGGCTCTGGAGTCAGAGCTGCAGCAGCTGCGGGCC
CGGCTCCAGGGGCTGGAGGCCGACTGTGTCCGGGGCCCAGATGGGGTGTGCCTCAGTGGGGATAGAGG
CCCACAGGGTGACAAGGCCATCAGGGAGCAAGGCCCCAGGGAGCAGGAGCCAGAACTCAGCTTCCTGA
AGCAGAAGGAACAGCTGGAGGCTGAGGCACAGGCATTAAGGCAAGAGTTAGAGAGGCAGCGACGGCTG
CTGGGGTCTGTACAGCAGGATCTGGAGAGGAGCTTGCAGGATGCCAGCCGCGGGGACCCAGCTCATGC
TGGCTTGGCTGAGCTGGGCCACAGATTGGCCCAGAAACTGCAGGGCCTGGAGAACTGGGGCCAGGACC
CTGGGGTCTCTGCCAATGCCTCAAAGGCCTGGCACCAGAAGTCCCACTTCCAGAATTCTAGGGAGTGG
AGTGGAAAGGAAAAGTGGTGGGATGGGCAGAGAGACCGGAAGGCTGAGCACTGGAAACATAAGAAGGA
AGAATCTGGCCGGGAAAGGAAGAAGAACTGGGGAGGTCAGGAGGACAGGGAGCCAGCAGGAAGGTGGA
AGGAGGGCAGGCCAAGGGTGGAGGAGTCGGGGAGCAAGAAGGAGGGCAAGCGACAGGGCCCGAAGGAA
CCCCCAAGGAAAAGTGGTAGCTTCCACTCCTCTGGAGAAAAGCAGAAGCAACCTCGGTGGAGGGAAGG
GACTAAGGACAGCCATGACCCCCTGCCATCCTGGGCAGAGCTGTTGAGGCCCAAGTACCGGGCACCCC
AGGGCTGCTCAGGTGTGGACGAGTGTGCCCGGCAGGAGGGCTTGACTTTCTTTGGCACAGAGCTAGTC
CCAGTGCGGCAACAGGAGCTGGCCTCTCTGCTAAGAACATACTTGGCACGGCTGCCCTGGGCTGGGCA
GCTGACCAAGGAGCTACCCCTCTCACCTGCTTTCTTTGGTGAGGATGGCATCTTCCGTCATGACCGCC
TCCGCTTCCGGGATTTTGTGGATGCCCTGGAGGACAGCTTGGAGGAGGTGGCTGTGCAACAGACAGGT
GATGATGATGAAGTAGATGACTTTGAGGACTTCATCTTCAGCCACTTCTTTGGAGACAAAGCACTGAA
GAAGAGGCCTCTTCCTCCCCACAGGTCAGGGAAGAAGGACAAGCACTCACAGAGCCCAAGAGCTGCGG
GGCCCAGGGAGGGGCACAGCCATAGCCACCACCACCACCACCGGGGCTGACACCCTGCCCCACAGGGA
ATGGCCTTGGCCTGGCCCAGCCCAAGATCCCAGCGTTATCCAACTCCTGGAGGGTGGACTCTGTCCTG
GCTTGTTTGGTGTCCTCAGATAT

| | | | |
|---|---|---|---|
| NOV16i, SNP13374627 | SEQ ID NO: 450 | 737 aa | MW at 81426.5kD |
| of CG52171-04, | SNP Pos: 608 | | SNP Change: |
| Protein Sequence | | | Ala to Val |

MASCPDSDNSWVLAGSESLPVETLGPASRMDPESERALQAPHSPSKTDGKELAGTMDGEGTLFQTESP
QSGSILTEETEVKGTLEGDVCGVEPPGPGDTVVQGDLQETTVVTGLGPDTQDLEGQSPPQSLPSTPKA
AWIREEGRCSSSDDDTDVDMEGLRRRRGREAGPPQPMVPLAVENQAGGEGAGGELGISLNMCLLGALV
LLGLGVLLFSGGLSESETGPMEEVERQVLPDPEVLEAVGDRQDGLREQLQAPVPPDSVPSLQNMGLLL
DKLAKENQDIRLLQAQLQAQKEELQSLMHQPKGLEEENAQLRGALQQGEAFQRALESELQQLRARLQG
LEADCVRGPDGVCLSGDRGPQGDKAIREQGPREQEPELSFLKQKEQLEAEAQALRQELERQRRLLGSV
QQDLERSLQDASRGDPAHAGLAELGHRLAQKLQGLENWGQDPGVSANASKAWHQKSHFQNSREWSGKE
KWWDGQRDRKAEHWKHKKEESGRERKKNWGGQEDREPAGRWKEGRPRVEESGSKKEGKRQGPKEPPRK
SGSFHSSGEKQKQPRWREGTKDSHDPLPSWAELLRPKYRAPQGCSGVDECARQEGLTFFGTELVPVRQ
QELASLLRTYLARLPWAGQLTKELPLSPAFFGEDGIFRHDRLRFRDFVDALEDSLEEVAVQQTGDDDE
VDDFEDFIFSHFFGDKALKKRPLPPHRSGKKDKHSQSPRAAGPREGHSHSHHHHHRG

| | | |
|---|---|---|
| NOV16j, SNP13374626 | SEQ ID NO: 451 | 2403 bp |
| of CG52171-04, | ORF Start: ATG at 81 | ORF Stop: TGA at 2292 |
| DNA Sequence | SNP Pos: 1918 | SNP Change:A to G |

GGCACGAGAAGTTAAGCTGAAGACCGAAGCAAGAGCTGGTTCAGGTGGCAGCCACAGCAGCCTCAGGG
ACCTCAGCAACTATGGCCTCCTGCCCAGACTCTGATAATAGCTGGGTGCTTGCTGGCTCCGAGAGCCT
GCCAGTGGAGACACTGGGCCCGGCATCCAGGATGGACCCAGAATCTGAGAGAGCCCTGCAGGCCCCTC
ACAGCCCCTCCAAGACAGATGGGAAAGAATTAGCTGGGACCATGGATGGAGAAGGGACGCTCTTCCAG
ACTGAAAGCCCTCAGTCTGGCAGCATTCTAACAGAGGAGACTGAGGTCAAGGGCACCCTGGAAGGTGA
TGTTTGTGGTGTGGAGCCTCCTGGCCCAGGAGACACAGTAGTCCAGGGAGACCTGCAGGAGACCACCG
TGGTGACAGGCCTGGGACCAGACACACAGGACCTGGAAGGCCAGAGCCCTCCACAGAGCCTGCCTTCA
ACCCCCAAAGCAGCTTGGATCAGGGAGGAGGGCCGCTGCTCCAGCAGTGACGATGACACCGACGTGGA
CATGGAGGGTCTGCGGAGACGGCGGGGCCGGGAGGCCGGCCCACCTCAGCCCATGGTGCCCCTGGCTG
TGGAGAACCAGGCTGGGGGTGAGGGTGCAGGCGGGGAGCTGGGCATCTCCCTCAACATGTGCCTCCTT
GGGGCCCTGGTTCTGCTTGGCCTGGGGGTCCTCCTCTTCTCAGGTGGCCTCTCAGAGTCTGAGACTGG
GCCCATGGAGGAAGTGGAGCGGCAGGTCCTCCCAGACCCCGAGGTGCTGGAAGCTGTGGGGGACAGGC
AGGATGGGCTAAGGGAACAGCTGCAGGCCCCAGTGCCTCCTGACAGTGTCCCCAGCCTGCAAAACATG
GGTCTTCTGCTGGACAAGCTGGCCAAGGAGAACCAGGACATCCGGCTGCTGCAGGCCCAGCTGCAGGC
CCAAAAGGAAGAGCTTCAGAGCCTGATGCACCAGCCCAAAGGGCTAGAGGAGGAGAATGCCCAGCTCC
GGGGGGCTCTGCAGCAGGGCGAAGCCTTCCAGCGGGCTCTGGAGTCAGAGCTGCAGCAGCTGCGGGCC
CGGCTCCAGGGGCTGGAGGCCGACTGTGTCCGGGGCCCAGATGGGGTGTGCCTCAGTGGGGATAGAGG
CCCACAGGGTGACAAGGCCATCAGGGAGCAAGGCCCCAGGGAGCAGGAGCCAGAACTCAGCTTCCTGA
AGCAGAAGGAACAGCTGGAGGCTGAGGCACAGGCATTAAGGCAAGAGTTAGAGAGGCAGCGACGGCTG
CTGGGGTCTGTACAGCAGGATCTGGAGAGGAGCTTGCAGGATGCCAGCCGCGGGGACCCAGCTCATGC
TGGCTTGGCTGAGCTGGGCCACAGATTGGCCCAGAAACTGCAGGGCCTGGAGAACTGGGGCCAGGACC
CTGGGGTCTCTGCCAATGCCTCAAAGGCCTGGCACCAGAAGTCCCACTTCCAGAATTCTAGGGAGTGG
AGTGGAAAGGAAAAGTGGTGGGATGGGCAGAGAGACCGGAAGGCTGAGCACTGGAAACATAAGAAGGA
AGAATCTGGCCGGGAAAGGAAGAAGAACTGGGGAGGTCAGGAGGACAGGGAGCCAGCAGGAAGGTGGA
AGGAGGGCAGGCCAAGGGTGGAGGAGTCGGGGAGCAAGAAGGAGGGCAAGCGACAGGGCCCGAAGGAA
CCCCCAAGGAAAAGTGGTAGCTTCCACTCCTCTGGAGAAAAGCAGAAGCAACCTCGGTGGAGGGAAGG
GACTAAGGACAGCCATGACCCCCTGCCATCCTGGGCAGAGCTGTTGAGGCCCAAGTACCGGGCACCCC
AGGGCTGCTCAGGTGTGGACGAGTGTGCCCGGCAGGAGGGCTTGACTTTCTTTGGCACAGAGCTAGCC
CCAGTGCGGCAACGGGAGCTGGCCTCTCTGCTAAGAACATACTTGGCACGGCTGCCCTGGGCTGGGCA
GCTGACCAAGGAGCTACCCCTCTCACCTGCTTTCTTTGGTGAGGATGGCATCTTCCGTCATGACCGCC
TCCGCTTCCGGGATTTTGTGGATGCCCTGGAGGACAGCTTGGAGGAGGTGGCTGTGCAACAGACAGGT
GATGATGATGAAGTAGATGACTTTGAGGACTTCATCTTCAGCCACTTCTTTGGAGACAAAGCACTGAA
GAAGAGGCCTCTTCCTCCCCACAGGTCAGGGAAGAAGGACAAGCACTCACAGAGCCCAAGAGCTGCGG
GGCCCAGGGAGGGGCACAGCCATAGCCACCACCACCACCACCGGGGCTGACACCCTGCCCCACAGGGA
ATGGCCTTGGCCTGGCCCAGCCCAAGATCCCAGCGTTATCCAACTCCTGGAGGGTGGACTCTGTCCTG
GCTTGTTTGGTGTCCTCAGATAT

| | | | |
|---|---|---|---|
| NOV16j, SNP13374626 | SEQ ID NO: 452 | 737 aa | MW at 81426.5kD |
| of CG52171-04, | SNP Pos: 613 | | SNP Change: |
| Protein Sequence | | | Gln to Arg |

MASCPDSDNSWVLAGSESLPVETLGPASRMDPESERALQAPHSPSKTDGKELAGTMDGEGTLFQTESP
QSGSILTEETEVKGTLEGDVCGVEPPGPGDTVVQGDLQETTVVTGLGPDTQDLEGQSPPQSLPSTPKA
AWIREEGRCSSSDDDTDVDMEGLRRRRGREAGPPQPMVPLAVENQAGGEGAGGELGISLNMCLLGALV
LLGLGVLLFSGGLSESETGPMEEVERQVLPDPEVLEAVGDRQDGLREQLQAPVPPDSVPSLQNMGLLL
DKLAKENQDIRLLQAQLQAQKEELQSLMHQPKGLEEENAQLRGALQQGEAFQRALESELQQLRARLQG
LEADCVRGPDGVCLSGDRGPQGDKAIREQGPREQEPELSFLKQKEQLEAEAQALRQELERQRRLLGSV
QQDLERSLQDASRGDPAHAGLAELGHRLAQKLQGLENWGQDPGVSANASKAWHQKSHFQNSREWSGKE
KWWDGQRDRKAEHWKHKKEESGRERKKNWGGQEDREPAGRWKEGRPRVEESGSKKEGKRQGPKEPPRK
SGSFHSSGEKQKQPRWREGTKDSHDPLPSWAELLRPKYHAPQGCSGVDECARQEGLTFFGTELAPVRQ
RELASLLRTYLARLPWAGQLTKELPLSPAFFGEDGIFRHDRLRFRDFVDALEDSLEEVAVQQTGDDDE
VDDFEDFIFSHFFGDKALKKRPLPPHRSGKKDKHSQSPRAAGPREGHSHSHHHHHRG

| | | |
|---|---|---|
| NOV16k, SNP13374625 | SEQ ID NO: 453 | 2403 bp |
| of CG52171-04, | ORF Start: ATG at 81 | ORF Stop: TGA at 2292 |
| DNA Sequence | SNP Pos: 1951 | SNP Change: C to T |

GGCACGAGAAGTTAAGCTGAAGACCGAAGCAAGAGCTGGTTCAGGTGGCAGCCACAGCAGCCTCAGGG
ACCTCAGCAACTATGGCCTCCTGCCCAGACTCTQATAATAGCTGGGTGCTTGCTGGCTCCGAGAGCCT
GCCAGTGGAGACACTGGGCCCGGCATCCAGGATGGACCCAGAATCTGAGAGAGCCCTGCAGGCCCCTC
ACAGCCCCTCCAAGACAGATGGGAAAGAATTAGCTGGGACCATGGATGGAGAAGGGACGCTCTTCCAG
ACTGAAAGCCCTCAGTCTGGCAGCATTCTAACAGAGGAGACTGAGGTCAAGGGCACCCTGGAAGGTGA
TGTTTGTGGTGTGGAGCCTCCTGGCCCAGGAGACACAGTAGTCCAGGGAGACCTGCAGGAGACCACCG
TGGTGACAGGCCTGGGACCAGACACACAGGACCTGGAAGGCCAGAGCCCTCCACAGAGCCTGCCTTCA
ACCCCCAAAGCAGCTTGGATCAGGGAGGAGGGCCGCTGCTCCAGCAGTGACGATGACACCGACGTGGA
CATGGAGGGTCTGCGGAGACGGCGGGGCCGGGAGGCCGGCCCACCTCAGCCCATGGTGCCCCTGGCTG
TGGAGAACCAGGCTGGGGGTGAGGGTGCAGGCGGGGAGCTGGGCATCTCCCTCAACATGTGCCTCCTT
GGGGCCCTGGTTCTGCTTGGCCTGGGGGTCCTCCTCTTCTCAGGTGGCCTCTCAGAGTCTGAGACTGG
GCCCATGGAGGAAGTGGAGCGGCAGGTCCTCCCAGACCCCGAGGTGCTGGAAGCTGTGGGGGACAGGC
AGGATGGGCTAAGGGAACAGCTGCAGGCCCCAGTGCCTCCTGACAGTGTCCCCAGCCTGCAAAACATG
GGTCTTCTGCTGGACAAGCTGGCCAAGGAGAACCAGGACATCCGGCTGCTGCAGGCCCAGCTGCAGGC
CCAAAAGGAAGAGCTTCAGAGCCTGATGCACCAGCCCAAAGGGCTAGAGGAGGAGAATGCCCAGCTCC
GGGGGGCTCTGCAGCAGGGCGAAGCCTTCCAGCGGGCTCTGGAGTCAGAGCTGCAGCAGCTGCGGGCC
CGGCTCCAGGGGCTGGAGGCCGACTGTGTCCGGGGCCCAGATGGGGTGTGCCTCAGTGGGGATAGAGG
CCCACAGGGTGACAAGGCCATCAGGGAGCAAGGCCCCAGGGAGCAGGAGCCAGAACTCAGCTTCCTGA
AGCAGAAGGAACAGCTGGAGGCTGAGGCACAGGCATTAAGGCAAGAGTTAGAGAGGCAGCGACGGCTG
CTGGGGTCTGTACAGCAGGATCTGGAGAGGAGCTTGCAGGATGCCAGCCGCGGGGACCCAGCTCATGC
TGGCTTGGCTGAGCTGGGCCACAGATTGGCCCAGAAACTGCAGGGCCTGGAGAACTGGGGCCAGGACC
CTGGGGTCTCTGCCAATGCCTCAAAGGCCTGGCACCAGAAGTCCCACTTCCAGAATTCTAGGGAGTGG
AGTGGAAAGGAAAAGTGGTGGGATGGGCAGAGAGACCGGAAGGCTGAGCACTGGAAACATAAGAAGGA
AGAATCTGGCCGGGAAAGGAAGAAGAACTGGGGAGGTCAGGAGGACAGGGAGCCAGCAGGAAGGTGGA
AGGAGGGCAGGCCAAGGGTGGAGGAGTCGGGGAGCAAGAAGGAGGGCAAGCGACAGGGCCCGAAGGAA
CCCCCAAGGAAAAGTGGTAGCTTCCACTCCTCTGGAGAAAAGCAGAAGCAACCTCGGTGGAGGGAAGG
GACTAAGGACAGCCATGACCCCCTGCCATCCTGGGCAGAGCTGTTGAGGCCCAAGTACCGGGCACCCC
AGGGCTGCTCAGGTGTGGACGAGTGTGCCCGGCAGGAGGGCTTGACTTTCTTTGGCACAGAGCTAGCC
CCAGTGCGGCAACAGGAGCTGGCCTCTCTGCTAAGAACATACTTGGTACGGCTGCCCTGGGCTGGGCA
GCTGACCAAGGAGCTACCCCTCTCACCTGCTTTCTTTGGTGAGGATGGCATCTTCCGTCATGACCGCC
TCCGCTTCCGGGATTTTGTGGATGCCCTGGAGGACAGCTTGGAGGAGGTGGCTGTGCAACAGACAGGT
GATGATGATGAAGTAGATGACTTTGAGGACTTCATCTTCAGCCACTTCTTTGGAGACAAAGCACTGAA
GAAGAGGCCTCTTCCTCCCCACAGGTCAGGGAAGAAGGACAAGCACTCACAGAGCCCAAGAGCTGCGG
GGCCCAGGGAGGGGCACAGCCATAGCCACCACCACCACCACCGGGGCTGACACCCTGCCCCACAGGGA
ATGGCCTTGGCCTGGCCCAGCCCAAGATCCCAGCGTTATCCAACTCCTGGAGGGTGGACTCTGTCCTG
GCTTGTTTGGTGTCCTCAGATAT

| | | | |
|---|---|---|---|
| NOV16k, SNP13374625 | SEQ ID NO: 454 | 737 aa | MW at 81426.5kD |
| of CG52171-04, | SNP Pos: 624 | | SNP Change: |
| Protein Sequence | | | Ala to Val |

MASCPDSDNSWVLAGSESLPVETLGPASRMDPESERALQAPHSPSKTDGKELAGTMDGEGTLFQTESP
QSGSILTEETEVKGTLEGDVCGVEPPGPGDTVVQGDLQETTVVTGLGPDTQDLEGQSPPQSLPSTPKA
AWIREEGRCSSSDDDTDVDMEGLRRRRGREAGPPQPMVPLAVENQAGGEGAGGELGISLNMCLLGALV
LLGLGVLLFSGGLSESETGPMEEVERQVLPDPEVLEAVGDRQDGLREQLQAPVPPDSVPSLQNMGLLL
DKLAKENQDIRLLQAQLQAQKEELQSLMHQPKGLEEENAQLRGALQQGEAFQRALESELQQLRARLQG
LEADCVRGPDGVCLSGDRGPQGDKAIREQGPREQEPELSFLKQKEQLEAEAQALRQELERQRRLLGSV
QQDLERSLQDASRGDPAHAGLAELGHRLAQKLQGLENWGQDPGVSANASKAWHQKSHFQNSREWSGKE
KWWDGQRDRKAEHWKHKKEESGRERKKNWGGQEDREPAGRWKEGRPRVEESGSKKEGKRQGPKEPPRK
SGSFHSSGEKQKQPRWREGTKDSHDPLPSWAELLRPKYRAPQGCSGVDECARQEGLTFFGTELAPVRQ
QELASLLRTYLVRLPWAGQLTKELPLSPAFFGEDGIFRHDRLRFRDFVDALEDSLEEVAVQQTGDDDE
VDDFEDFIFSHFFGDKALKKRPLPPHRSGKKDKHSQSPRAAGPREGHSHSHHHHHRG

| | | |
|---|---|---|
| NOV16l, SNP13374624 | SEQ ID NO: 455 | 2403 bp |
| of CG52171-04, | ORF Start: ATG at 81 | ORF Stop: TGA at 2292 |
| DNA Sequence | SNP Pos: 2124 | SNP Change: G to T |

GGCACGAGAAGTTAAGCTGAAGACCGAAGCAAGAGCTGGTTCAGGTGGCAGCCACAGCAGCCTCAGGC
ACCTCAGCAACTATGGCCTCCTGCCCAGACTCTGATAATAGCTGGGTGCTTGCTGGCTCCGAGAGCCT
GCCAGTGGAGACACTGGGCCCGGCATCCAGGATGGACCCAGAATCTGAGAGAGCCCTGCAGGCCCCTC
ACAGCCCCTCCAAGACAGATGGGAAAGAATTAGCTGGGACCATGGATGGAGAAGGGACGCTCTTCCAG
ACTGAAAGCCCTCAGTCTGGCAGCATTCTAACAGAGGAGACTGAGGTCAAGGGCACCCTGGAAGGTGA
TGTTTGTGGTGTGGAGCCTCCTGGCCCAGGAGACACAGTAGTCCAGGGAGACCTGCAGGAGACCACCG
TGGTGACAGGCCTGGGACCAGACACACAGGACCTGGAAGGCCAGAGCCCTCCACAGAGCCTGCCTTCA
ACCCCCAAAGCAGCTTGGATCAGGGAGGAGGGCCGCTGCTCCAGCAGTGACGATGACACCGACGTGGA
CATGGAGGGTCTGCGGAGACGGCGGGGCCGGGAGGCCGGCCCACCTCAGCCCATGGTGCCCCTGGCTG
TGGAGAACCAGGCTGGGGGTGAGGGTGCAGGCGGGGAGCTGGGCATCTCCCTCAACATGTGCCTCCTT
GGGGCCCTGGTTCTGCTTGGCCTGGGGGTCCTCCTCTTCTCAGGTGGCCTCTCAGAGTCTGAGACTGG
GCCCATGGAGGAAGTGGAGCGGCAGGTCCTCCCAGACCCCGAGGTGCTGGAAGCTGTGGGGGACAGGC
AGGATGGGCTAAGGGAACAGCTGCAGGCCCCAGTGCCTCCTGACAGTGTCCCCAGCCTGCAAAACATG
GGTCTTCTGCTGGACAAGCTGGCCAAGGAGAACCAGGACATCCGGCTGCTGCAGGCCCAGCTGCAGGC
CCAAAAGGAAGAGCTTCAGAGCCTGATGCACCAGCCCAAAGGGCTAGAGGAGGAGAATGCCCAGCTCC
GGGGGGCTCTGCAGCAGGGCGAAGCCTTCCAGCGGGCTCTGGAGTCAGAGCTGCAGCAGCTGCGGGCC
CGGCTCCAGGGGCTGGAGGCCGACTGTGTCCGGGGCCCAGATGGGGTGTGCCTCAGTGGGGATAGAGG
CCCACAGGGTGACAAGGCCATCAGGGAGCAAGGCCCCAGGGAGCAGGAGCCAGAACTCAGCTTCCTGA
AGCAGAAGGAACAGCTGGAGGCTGAGGCACAGGCATTAAGGCAAGAGTTAGAGAGGCAGCGACGGCTG
CTGGGGTCTGTACAGCAGGATCTGGAGAGGAGCTTGCAGGATGCCAGCCGCGGGGACCCAGCTCATGC
TGGCTTGGCTGAGCTGGGCCACAGATTGGCCCAGAAACTGCAGGGCCTGGAGAACTGGGGCCAGGACC
CTGGGGTCTCTGCCAATGCCTCAAAGGCCTGGCACCAGAAGTCCCACTTCCAGAATTCTAGGGAGTGG
AGTGGAAAGGAAAAGTGGTGGGATGGGCAGAGAGACCGGAAGGCTGAGCACTGGAAACATAAGAAGGA
AGAATCTGGCCGGGAAAGGAAGAAGAACTGGGGAGGTCAGGAGGACAGGGAGCCAGCAGGAAGGTGGA
AGGAGGGCAGGCCAAGGGTGGAGGAGTCGGGGAGCAAGAAGGAGGGCAAGCGACAGGGCCCGAAGGAA
CCCCCAAGGAAAAGTGGTAGCTTCCACTCCTCTGGAGAAAAGCAGAAGCAACCTCGGTGGAGGGAAGG
GACTAAGGACAGCCATGACCCCCTGCCATCCTGGGCAGAGCTGTTGAGGCCCAAGTACCGGGCACCCC
AGGGCTGCTCAGGTGTGGACGAGTGTGCCCGGCAGGAGGGCTTGACTTTCTTTGGCACAGAGCTAGCC
CCAGTGCGGCAACAGGAGCTGGCCTCTCTGCTAAGAACATACTTGGCACGGCTGCCCTGGGCTGGGCA
GCTGACCAAGGAGCTACCCCTCTCACCTGCTTTCTTTGGTGAGGATGGCATCTTCCGTCATGACCGCC
TCCGCTTCCGGGATTTTGTGGATGCCCTGGAGGACAGCTTGGAGGAGGTGGCTGTGCAACAGACAGGT
GATGATGATGAAGTATATGACTTTGAGGACTTCATCTTCAGCCACTTCTTTGGAGACAAAGCACTGAA
GAAGAGGCCTCTTCCTCCCCACAGGTCAGGGAAGAAGGACAAGCACTCACAGAGCCCAAGAGCTGCGG
GGCCCAGGGAGGGGCACAGCCATAGCCACCACCACCACCACCGGGGCTGACACCCTGCCCCACAGGGA
ATGGCCTTGGCCTGGCCCAGCCCAAGATCCCAGCGTTATCCAACTCCTGGAGGGTGGACTCTGTCCTG
GCTTGTTTGGTGTCCTCAGATAT

| | | | |
|---|---|---|---|
| NOV16l, SNP13374624 | SEQ ID NO: 456 | 737 aa | MW at 81446.5kD |
| of CG52171-04, | SNP Pos: 682 | | SNP Change: |
| Protein Sequence | | | Asp to Tyr |

MASCPDSDNSWVLAGSESLPVETLGPASRMDPESEHALQAPHSPSKTDGKELAGTMDGEGTLFQTESP
QSGSILTEETEVKGTLEGDVCGVEPPGPGDTVVQGDLQETTVVTGLGPDTQDLEGQSPPQSLPSTPKA
AWIREEGRCSSSDDDTDVDMEGLRRRRGREAGPPQPMVPLAVENQAGGEGAGGELGISLNMCLLGALV
LLGLGVLLFSGGLSESETGPMEEVERQVLPDPEVLEAVGDRQDGLREQLQAPVPPDSVPSLQNMGLLL
DKLAKENQDIRLLQAQLQAQKEELQSLMHQPKGLEEENAQLRGALQQGEAFQRALESELQQLRARLQG
LEADCVRGPDGVCLSGDRGPQGDKAIREQGPREQEPELSFLKQKEQLEAEAQALRQELERQRRLLGSV
KWWDGQRDRKAEHWKHKKEESGRERKKNWGGQEDREPAGRWKEGRPRVEESGSKKEGKRQGPKEPPRK
SGSFHSSGEKQKQPRWREGTKDSHDPLPSWAELLRPKYRAPQGCSGVDECARQEGLTFFGTELAPVRQ
QELASLLRTYLARLPWAGQLTKELPLSPAFFGEDGIFRHDRLRFRDFVDALEDSLEEVAVQQTGDDDE
VYDFEDFIFSHFFGDKALKKRPLPPHRSGKKDKHSQSPRAAGPREGHSHSHHHHHRG

A ClustalW comparison of the above protein sequences yields the following sequence alignment shown in Table 16B.

TABLE 16B
Comparison of the NOV16 protein sequences.

| | |
|---|---|
| NOV16a | MASCPDSDNSWVLAGSESLPVETLGPASRMDPESERALQAPHSPSKTDGKELAGTNDGEG |
| NOV16b | ------------------------------------------------------------ |
| NOV16c | MASCPDSDNSWVLAGSESLPVETLGPASRMDPESERALQAPHSPSKTDGKELAGTMDGEG |
| NOV16d | MASCPDSDNSWVLAGSESLPVETLGPASRMDPESERALQAPHSPSKTDGKELAGTMDGEG |
| NOV16e | ------------------------------------------------------------ |
| NOV16f | MASCPDSDNSWVLAGSESLPVETLGPASRMDPESERALQAPHSPSKTDGKELAGTMDGEG |
| NOV16a | TLFQTESPQSGSILTEETEVKGTLEGDVCGVEPPGPGDTVVQGDLQETTVVTGLGPDTQD |
| NOV16b | ------------------------------------------------------------ |
| NOV16c | TLFQTESPQSGSILTEETEVKGTLEGDVCGVEPPGPGDTVVQGDLQETTVVTGLGPDTQD |
| NOV16d | TLFQTESPQSGSILTEETEVKGTLEGDVCGVEPPGPGDTVVQGDLQETTVVTGLGPDTQD |
| NOV16e | ------------------------------------------------------------ |
| NOV16f | TLFQTESPQSGSILTEETEVKGTLEGDVCGVEPPGPGDTVVQGDLQETTVVTGLGPDTQD |
| NOV16a | LEGQSPPQSLPSTPKAAWIREEGRCSSSDDDTDVDMEGLRRRRGREAGPPQPMVPLAVEN |
| NOV16b | ------------------------------------------------------------ |
| NOV16c | LEGQSPPQSLPSTPKAAWIREEGRCSSSDDDTDVDMEGLRRRRGREAGPPQPMVPLAVEN |
| NOV16d | LEGQSPPQSLPSTPKAAWIREEGRCSSSDDDTDVDMEGLRRPRGREAGPPQPMVPLAVEN |
| NOV16e | ------------------------------------------------------------ |
| NOV16f | LEGQSPPQSLPSTPKAAWIREEGRCSSSDDDTDVDMEGLRRRRGREAGPPQPMVPLAVEN |
| NOV16a | QAGGEGAGGELGISLNMCLLGALVLLGLGVLLFSGGLSESETGPMEEVERQVLPDPEVLE |
| NOV16b | ----------------MCLLGALVLLGLGVLLFSGGLSESETGPMEEVERQVLPDPEVLE |
| NOV16c | QAGGEGAGGELGISLNMCLLGALGLLGLGVLLFSGGLSESETGPMEEVERQVLPDPEVLE |
| NOV16d | QAGGEGAGGELGISLNMCLLGALGLLGLGVLLFSGGLSESETGPMEEVERQVLPDPEVLE |
| NOV16e | --------------------------------------ESETGPMEEVERQVLPDPEVLE |
| NOV16f | QAGGEGAGGELGISLNMCLLGALVLLGLGVLLFSGGLSESETGPMEEVERQVLPDPEVLE |
| NOV16a | AVGDRQDGLREQLQAPVPPDSVPSLQNMGLLLDKLAKENQDIRLLQAQLQAQKEELQSLM |
| NOV16b | AVGDRQDGLREQLQAPVPPDSVPSLQNMGLLLDKLAKENQDIRLLQAQLQAQKEELQSLM |
| NOV16c | AVGDRQDGLREQLQAPVPPDSVPSLQNMGLLLDKLAKENQDIRLLQAQLQAQKEELQSLM |
| NOV16d | AVGDRQDGLREQLQAPVPPDSVPSLQNMGLLLDKLAKENQDIRLLQAQLQAQKEELQSLM |
| NOV16e | AVGDRQDGLREQLQAPVPPDSVPSLQNMGLLLDKLAKENQDIRLLQAQLQAQKEELQSLM |
| NOV16f | AVGDRQDGLREQLQAPVPPDSVPSLQNMGLLLDKLAKENQDIRLLQAQLQAQKEELQSLM |
| NOV16a | HQPKGLEEENAQLRGALQQGEAFQRALESELQQLRARLQGLEADCVRGPDGVCLSGDRGP |
| NOV16b | HQPKGLEEENAQLRGALQQGEAFQRALESELQQLRARLQGLEADCVRGPDGVCLSGGRGP |
| NOV16c | HQPKGLEEENAQLRGALQQGEAFQRALESELQQLRARLQGLEADCVRGPD---------- |
| NOV16d | HQPKGLEEENAQLRGALQQGEAFQRALESELQQLRARLQGLEADCVRGPDGVCLSGGRGP |
| NOV16e | HQPKGLEEENAQLRGALQQGEAFQRALESELQQLRARLQGLEADCVRGPDGVCLSGDRGP |
| NOV16f | HQPKGLEEENAQLRGALQQGEAFQRALESELQQLRARIQGLEADCVRGPDGVCLSGDRGP |
| NOV16a | QGDKAIREQGPREQEPELSFLKQKEQLEAEAQALRQELERQRRLLGSVQQDLERSLQDAS |
| NOV16b | QGDKAIREQGPREQEPELSFLKQKEQLEAEAQALRQELERQRRLLGSVQQDLERSLQDAS |
| NOV16c | ------------------------------------------------------------ |
| NOV16d | QGDKAIREQGPREQEPELSFLKQKEQLEAEAQALRQELERQRRLLGSVQQDLERSLQDAS |
| NOV16e | QGDKAIREQGPREQEPELSFLKQKEQLEAEAQALRQELERQRRLLGSVQQDLERSLQDAS |
| NOV16f | QGDKAIREQGPREQEPELSFLNQKEQLEAEAQALRQELERQRRLLGSVQQDLERSLQDAS |
| NOV16a | RGDPAHAGLAELGHRLAQKLQGLENWGQDPGVSANASKAWHQKSHFQNSREWSGKEKWWD |
| NOV16b | RGDPAHAGLAELGHRLAQKLQGLENWGQDPGVSANASKAWHQKSHFQNSREWSGKEKWWD |
| NOV16c | ------------------------------------------------------------ |
| NOV16d | RGDPAHAGLAELGHRLAQKLQGLENWGQDPGVSANASKAWHQKSHFQNSREWSGKEKWWD |
| NOV16e | RGDPAHAGLAELGHRLAQKLQGLENWGQDPGVSANASKAWHQKSHFQNSREWSGKEKWWD |
| NOV16f | RGDPAHAGLAELGHRLAQKLQGLENWGQDPGVSANASKAWHQKSHFQNSREWSGKEKWWD |
| NOV16a | GQRDRKAEHWKHKKEESGRERKKNWGGQEDREPAGRWKEGRPRVEESGSKKEGKRQGPKE |
| NOV16b | GQRDRKAEHWKHKKEESGRERKKNWGGQEDREPAGRWKEGRPRVEESGSKKEGKRQGPKE |
| NOV16c | ------------------------------------------------------------ |
| NOV16d | GQRDRKAEHWKHKKEESGRERKKNWGGQEDREPAGRWKEGRPRVEESGSKKEGKRQGPKE |
| NOV16e | GQRDRKAEHWKHKKEESGRERKKNWGGQEDREPAGRWKEGRPRVEESGSKKEGKRQGPKE |
| NOV16f | GQRDRKAEHWKHKKEESGRERKKNWGGQEDREPAGRWKEGRPRVEESGSKKEGKRQAPKE |
| NOV16a | PPRKSGSFHSSGEKQKQPRWREGTKDSHDPLPSWAELLRPKYRAPQGCSGVDECARQEGL |
| NOV16b | PPRKSGSFHSSGEKQKQPRWREGTKGSHDPLPSWAELLRPKYRAPQGCSGVDECARQEGL |
| NOV16c | ------------------------------------------------------------ |
| NOV16d | PPRKSGSFHSSGEKQKQPRWREGTKGSHDPLPSWAELLRPKYRAPQGCSGVDECARQEGL |
| NOV16e | PPRKSGSFHSSGEKQKQPRWREGTKDSHDPLPSWAELLRPKYRAPQGCSGVDECARQEGL |
| NOV16f | PPRKSGSFHSSGEKQKQPRWREGTKDSHDPLPSWAELLRPKYRAPQGCSGVDECARQEGL |
| NOV16a | TFFGTELAPVRQQELASLLRTYLARLPWAGQLTKELPLSPAFFGEDGIFRHDRLRFRDFV |
| NOV16b | TFFGTELAPVRQQELASLLRTYLARLPWAGQLTKELPLSPAFFGEDGIFRHDRLRFRDFV |
| NOV16c | --------------------------------------------GDGIFRHDRLRFRDFV |
| NOV16d | TFFGTELAPVRQQELASLLRTYLARLPWAGQLTKELPLSPAFFGEDGIFRHDRLRFRDFV |
| NOV16e | TFFGTELAPVRQQELASLLRTYLARLPWAGQLTKELPLSPAFFGEDGIFRHDRLRFRDFV |
| NOV16f | TFFGTELAPVRQQELASLLRTYLARLPWAGQLTKELPLSPAFFGEDGIFRHDRLRFRDFV |
| NOV16a | DALEDSLEEVAVQQTGDDDEVDDFEDFIFSHFFGDKALKKRPLPPHRSGKKDKHSQSPPA |
| NOV16b | DALEDSLEEVAVQQTGDDDEVDDFEDFIFSHFFGDKALKKR------SGKKDKHSQSPRA |
| NOV16c | DALEDSLEEVAVQQTGDDDEVDDFEDFIFSHFFGDKALKKR------SGKKDKHSQSPRA |
| NOV16d | DALEDSLEEVLEFSGR-------------------------------------------- |
| NOV16e | DALEDSLEEVAVQQTGDDDEVDDFEDFIFSHFFGDKALKKR------SGKKDKHSQSPRA |
| NOV16f | DALEDSLEEVAVQQTGDDDEVDDFEDFIFSHFFGDKALKKR------SGKKDKHSQSPPA |
| NOV16a | AGPREGHSHSHHHHHRG |
| NOV16b | AGPREGHSHSHHHHHRG |
| NOV16c | AGPREGHSHSHHHHHRG |
| NOV16d | ----------------- |
| NOV16e | AGPREGHSHSHHHHHRG |
| NOV16f | AGPREGHSHSHHHHRG- |
| NOV16a | (SEQ ID NO: 434) |
| NOV16b | (SEQ ID NO: 436) |
| NOV16c | (SEQ ID NO: 438) |
| NOV16d | (SEQ ID NO: 440) |
| NOV16e | (SEQ ID NO: 442) |
| NOV16f | (SEQ ID NO: 444) |

Further analysis of the NOV16a protein yielded the following properties shown in Table 16C.

TABLE 16C
Protein Sequence Properties NOV16a

| | |
|---|---|
| SignalP analysis: | No Known Signal Sequence Predicted |
| PSORT II analysis: | |

| PSG: | a new signal peptide prediction method | |
|---|---|---|
| | N-region: | length 8; pos. chg 0; neg. chg 2 |
| | H-region: | length 8; peak value 0.00 |
| | PSG score: | −4.40 |

| | |
|---|---|
| GvH: | von Heijne's method for signal seq. recognition |
| | GvH score (threshold: −2.1): −7.85 |
| | possible cleavage site: between 23 and 24 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 1 | | |
| | Number of TMS(s) for threshold 0.5: 1 | | |
| | INTEGRAL | Likelihood = | Transmembrane 197-213 |
| | | −10.46 | |

| | |
|---|---|
| PERIPHERAL | Likelihood = 9.34 (at 12) |

| | |
|---|---|
| | ALOM score: −10.46 (number of TMSs: 1) |
| MTOP: | Prediction of membrane topology (Hartmann et al.) |
| | Center position for calculation: 204 |
| | Charge difference: −1.0 C(−4.0)-N(−3.0) |
| | N >= C: N-terminal side will be inside |

>>> membrane topology: type 2 (cytoplasmic tail 1 to 197)

| MITDISC: | discrimination of mitochondrial targeting seq | | | |
|---|---|---|---|---|
| | R content: | 0 | Hyd Moment(75): | 2.09 |
| | Hyd Moment(95): | 1.02 | G content: | 0 |
| | D/E content: | 2 | S/T content: | 2 |

| | |
|---|---|
| | Score: −7.60 |
| Gavel: | prediction of cleavage sites for mitochondrial preseq |
| | cleavage site motif not found |

NUCDISC: discrimination of nuclear localization signals

| | |
|---|---|
| | pat4: RRRR (5) at 160 |
| | pat4: KHKK (3) at 491 |
| | pat4: KKRP (4) at 699 |
| | pat7: none |
| | bipartite: none |
| | content of basic residues: 12.9% |
| | NLS Score: 0.28 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 89 |
| COIL: | Lupas's algorithm to detect coiled-coil regions |
| 263 S | 0.81 |
| 264 L | 0.87 |
| 265 Q | 0.96 |
| 266 N | 0.97 |
| 267 M | 0.98 |
| 268 G | 0.99 |
| 269 L | 1.00 |
| 270 L | 1.00 |
| 271 L | 1.00 |
| 272 D | 1.00 |
| 273 K | 1.00 |
| 274 L | 1.00 |
| 275 A | 1.00 |
| 276 K | 1.00 |
| 277 E | 1.00 |
| 278 N | 1.00 |
| 279 Q | 1.00 |
| 280 D | 1.00 |
| 281 I | 1.00 |
| 282 R | 1.00 |
| 283 L | 1.00 |
| 284 L | 1.00 |
| 285 Q | 1.00 |
| 286 A | 1.00 |
| 287 Q | 1.00 |
| 288 L | 1.00 |
| 289 Q | 1.00 |
| 290 A | 1.00 |
| 291 Q | 1.00 |
| 292 K | 1.00 |
| 293 E | 1.00 |
| 294 E | 1.00 |
| 295 L | 1.00 |
| 296 Q | 1.00 |
| 297 S | 1.00 |
| 298 L | 1.00 |
| 299 M | 1.00 |
| 300 H | 1.00 |
| 301 Q | 1.00 |
| 303 K | 0.71 |
| 304 G | 0.71 |
| 305 L | 0.87 |
| 306 E | 0.87 |
| 307 E | 0.87 |
| 308 E | 0.87 |
| 309 N | 0.87 |
| 310 A | 0.87 |
| 311 Q | 0.87 |
| 312 L | 0.87 |
| 313 R | 0.87 |
| 314 G | 0.87 |
| 315 A | 0.90 |
| 316 L | 0.94 |
| 317 Q | 0.94 |
| 318 Q | 0.94 |
| 319 G | 0.94 |
| 320 E | 0.94 |
| 321 A | 0.94 |
| 322 F | 0.94 |
| 323 Q | 0.94 |
| 324 R | 0.94 |
| 325 A | 0.94 |
| 326 L | 0.94 |
| 327 E | 0.94 |
| 328 S | 0.94 |
| 329 E | 0.94 |
| 330 L | 0.94 |
| 331 Q | 0.94 |
| 332 Q | 0.94 |
| 333 L | 0.94 |
| 334 R | 0.94 |
| 335 A | 0.94 |
| 336 R | 0.94 |
| 337 L | 0.94 |
| 338 Q | 0.94 |
| 339 G | 0.94 |
| 340 L | 0.94 |
| 341 E | 0.94 |
| 342 A | 0.94 |
| 343 D | 0.94 |
| 344 C | 0.79 |
| 376 E | 0.95 |
| 377 L | 1.00 |
| 378 S | 1.00 |
| 379 F | 1.00 |
| 380 L | 1.00 |
| 381 K | 1.00 |
| 382 Q | 1.00 |
| 383 K | 1.00 |
| 384 E | 1.00 |
| 385 Q | 1.00 |
| 386 L | 1.00 |
| 387 E | 1.00 |
| 388 A | 1.00 |
| 389 E | 1.00 |
| 390 A | 1.00 |
| 391 Q | 1.00 |
| 392 A | 1.00 |
| 393 L | 1.00 |
| 394 R | 1.00 |
| 395 Q | 1.00 |
| 396 E | 1.00 |
| 397 L | 1.00 |
| 398 E | 1.00 |
| 399 R | 1.00 |
| 400 Q | 1.00 |
| 401 R | 1.00 |
| 402 R | 1.00 |
| 403 L | 1.00 |
| 404 L | 1.00 |
| 405 G | 1.00 |
| 406 S | 1.00 |
| 407 V | 1.00 |
| 408 Q | 1.00 |
| 409 Q | 1.00 |
| 410 D | 1.00 |
| 411 L | 1.00 |
| 412 E | 1.00 |
| 413 R | 1.00 |
| 414 S | 0.99 |
| 415 L | 0.94 |
| 416 Q | 0.94 |
| 417 D | 0.92 |
| 418 A | 0.92 |
| 419 S | 0.78 |
| 420 R | 0.78 |
| 421 G | 0.54 |
| | total: 127 residues |

Final Results (k = 9/23):

```
39.1%: nuclear
26.1%: mitochondrial
  8.7%: cytoplasmic
  8.7%: Golgi
  4.3%: plasma membrane
  4.3%: vesicles of secretory system
  4.3%: extracellular, including cell wall
  4.3%: peroxisomal
```

>> prediction for CG52171-04 is nuc (k = 23)

A search of the NOV16a protein against the Geneseq database, a proprietary database that contains sequences published in patents and patent publication, yielded several homologous proteins shown in Table 16D.

TABLE 16D
Geneseq Results for NOV16a

| | | NOV16a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB93999 | Human protein sequence SEQ ID | 30 . . . 737 | 700/708 (98%) | 0.0 |
| | NO: 14107 - Homo sapiens, 702 aa. | 1 . . . 702 | 700/708 (98%) | |
| | [EP1074617-A2, 07-FEB-2001] | | | |
| ABG20800 | Novel human diagnostic protein | 7 . . . 700 | 624/697 (89%) | 0.0 |
| | #20791 - Homo sapiens, 669 aa. | 2 . . . 668 | 634/697 (90%) | |
| | [WO200175067-A2, 11-OCT-2001] | | | |
| AAB95336 | Human protein sequence SEQ ID | 1 . . . 566 | 564/566 (99%) | 0.0 |
| | NO: 17611 - Homo sapiens, 662 aa. | 1 . . . 566 | 566/566 (99%) | |
| | [EP1074617-A2, 07-FEB-2001] | | | |
| AAB62402 | Human MBSP6 polypeptide (clone | 197 . . . 737 | 533/541 (98%) | 0.0 |
| | 3207791.0.128) - Homo sapiens, 535 | 1 . . . 535 | 533/541 (98%) | |
| | aa. [WO200127277-A2, 19-APR-2001] | | | |
| AAB80449 | Gene #23 associated peptide #1 - | 17 . . . 397 | 375/381 (98%) | 0.0 |
| | Homo sapiens, 451 aa. | 8 . . . 388 | 377/381 (98%) | |
| | [WO200107459-A1, 01-FEB-2001] | | | |

In a BLAST search of public sequence databases, the NOV16a protein was found to have homology to the proteins shown in the BLASTP data in Table 16E.

TABLE 16E
Public BLASTP Results for NOV16a

| | | NOV16a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9HD85 | Hematopoietic PBX-interacting | 1 . . . 737 | 730/737 (99%) | 0.0 |
| | protein - Homo sapiens (Human), | 1 . . . 731 | 730/737 (99%) | |
| | 731 aa. | | | |
| Q96AQ6 | Hematopoietic PBX-interacting | 1 . . . 737 | 730/737 (99%) | 0.0 |
| | protein - Homo sapiens (Human), | 1 . . . 731 | 730/737 (99%) | |
| | 731 aa. | | | |
| Q9HA02 | Hypothetical protein FLJ12435 - | 30 . . . 737 | 700/708 (98%) | 0.0 |
| | Homo sapiens (Human), 702 aa. | 1 . . . 702 | 700/708 (98%) | |
| Q9H8X6 | Hypothetical protein FLJ13157 - | 1 . . . 566 | 564/566 (99%) | 0.0 |
| | Homo sapiens (Human), 662 aa. | 1 . . . 566 | 566/566 (99%) | |
| CAC43890 | Sequence 11 from Patent | 197 . . . 737 | 533/541 (98%) | 0.0 |
| | WO0127277 - Homo sapiens | 1 . . . 535 | 533/541 (98%) | |
| | (Human), 535 aa. | | | |

PFam analysis predicts that the NOV16a protein contains the domains shown in the Table 16F.

TABLE 16F
Domain Analysis of NOV16a

| | | | |
|---|---|---|---|
| Pfam | NOV16a | Identities/ | Expect |
| Domain | Match Region | Similarities | Value |
| | | for the | |
| | | Matched Region | |

### Example 17.

The NOV17 clone was analyzed, and the nucleotide and encoded polypeptide sequences are shown in Table 17A.

TABLE 17A
NOV17 Sequence Analysis

| | | |
|---|---|---|
| NOV17a, CG52534-06 | SEQ ID NO: 457 | 1275 bp |
| DNA Sequence | ORF Start: ATG at 1 | ORF Stop: TGA at 1273 |

ATGGAGCGGCTTTGGGGTCTATTCCAGAGAGCGCAACAACTGTCCCCAAGATCCTCTCAGACCGTCTA
CCAGCGTGTGGAAGGCCCCCGGAAAGGGCACCTGGAGGAGGAAGAGGAAGACGGGGAGGAGGGGGCGG
AGACATTGGCCCACTTCTGCCCCATGGAGCTGAGGGGCCCTGAGCCCCTGGGCTCTAGACCCAGGCAG
CCAAACCTCATTCCCTGGGCGGCAGCAGGACGGAGGGCTGCCCCCTACCTGGTCCTGACGGCCCTGCT
GATCTTCACTGGGGCCTTCCTACTGGGCTACGTCGCCTTCCGAGGGTCCTGCCAGGCGTGCGGAGACT
CTGTGTTGGTGGTCAGTGAGGATGTCAACTATGAGCCTGACCTGGATTTCCACCAGGGCAGACTCTAC
TGGAGCGACCTCCAGGCCATGTTCCTGCAGTTCCTGGGGGAGGGGCGCCTGGAGGACACCATCAGGCA
AACCAGCCTTCGGGAACGGGTGGCAGGCTCGGCCGGGATGGCCGCTCTGACTCAGGACATTCGCGCGG
CGCTCTCCCGCCAGAAGCTGGACCACGTGTGGACCGACACGCACTACGTGGGGCTGCAATTCCCGGAT
CCGGCTCACCCCAACACCCTGCACTGGGTCGATGAGGCCGGGAAGGTCGGAGAGCAGCTGCCGCTGGA
GGACCCTGACGTCTACTGCCCCTACAGCGCCATCGGCAACGTCACGGGAGAGCTGGTGTACGCCCACT
ACGGGCGGCCCGAAGACCTGCAGGACCTGCGGGCCAGGGGCGTGGATCCAGTGGGCCGCCTGCTGCTG
GTGCGCGTGGGGGTGATCAGCTTCGCCCAGAAGGTGACCAATGCTCAGGACTTCGGGGCTCAAGGAGT
GCTCATATACCCAGAGCCAGCGGACTTCTCCCAGGACCCACCCAAGCCAAGCCTGTCCAGCCAGCAGG
CAGTGTATGGACATGTGCACCTGGGAACTGGAGACCCCTACACACCTGGCTTCCCTTCCTTCAATCAA
ACCCAGTTCCCTCCAGTTGCATCATCAGGCCTTCCCAGCATCCCAGCCCAGCCCATCAGTGCAGACAT
TGCCTCCCGCCTGCTGGACCACCTGCGGCTGCTGCGCTCCAACAGCTCCGGGACCCCCGGGGCCACCT
CCTCCACTGGCTTCCAGGAGAGCCGTTTCCGGCGTCAGCTAGCCCTGCTCACCTGGACGCTGCAAGGG
GCAGCCAATGCGCTTAGCGGGGATGTCTGGAACATTGATAACAACTTCTGA

| | | | |
|---|---|---|---|
| NOV17a, CG52534-06 | SEQ ID NO: 458 | 424aa | MW at 46603.7kD |
| Protein Sequence | | | |

MERLWGLFQRAQQLSPRSSQTVYQRVEGPRKGHLEEEEEDGEEGAETLAHFCPMELRGPEPLGSRPRQ
PNLIPWAAAGRRAAPYLVLTALLIFTGAFLLGYVAFRGSCQACGDSVLVVSEDVNYEPDLDFHQGRLY
WSDLQAMFLQFLGEGRLEDTIRQTSLRERVAGSAGMAALTQDIPAALSRQKLDHVWTDTHYVGLQFPD
PAHPNTLHWVDEAGKVGEQLPLEDPDVYCPYSAIGNVTGELVYAHYGRPEDLQDLRARGVDPVGRLLL
VRVGVISFAQKVTNAQDFGAQGVLIYPEPADFSQDPPKPSLSSQQAVYGHVHLGTGDPYTPGFPSFNQ
TQFPPVASSGLPSIPAQPISADIASRLLDHLRLLRSNSSGTPGATSSTGFQESRFRRQLALLTWTLQG
AANALSGDVWNIDNNF

| | | |
|---|---|---|
| NOV17b, CG52534-01 | SEQ ID NO: 459 | 2857 bp |
| DNA Sequence | ORF Start: ATG at 28 | ORF Stop: TGA at 1495 |

CTGCAGGCTTCAGGAGGGGACACAAGCATGGAGCGGCTTTGGGGTCTATTCCAGAGAGCGCAACAACT
GTCCCCAAGATCCTCTCAGACCGTCTACCAGCGTGTGGAAGGCCCCCGGAAAGGGCACCTGGAGGAGG
AAGAGGAAGACGGGGAGGAGGGGGCGGAGACATTGGCCCACTTCTGCCCCATGGAGCTGAGGGGCCCT
GAGCCCCTGGGCTCTAGACCCAGGCAGCCAAACCTCATTCCCTGGGCGGCAGCAGGACGGAGGGCTGC
CCCCTACCTGGTCCTGACGGCCCTGCTGATCTTCACTGGGGCCTTCCTACTGGGCTACGTCGCCTTCC
GAGGGTCCTGCCAGGCGTGCGGAGACTCTGTGTTGGTGGTCAGTGAGGATGTCAACTATGAGCCTGAC
CTGGATTTCCACCAGGGCAGACTCTACTGGAGCGACCTCCAGGCCATGTTCCTGCAGTTCCTGGGGGA
GGGGCGCCTGGAGGACACCATCAGGCAAACCAGCCTTCGGGAACGGGTGGCAGGCTCGGCCGGGATGG
CCGCTGCTGACTCAGGACATTCGCGCGGCCTCTCCCGCCAGAAGCTGGACCACGTGTGGACCGACACG
CACTACGTGGGGCTGCAATTCCCGGATCCGGCTCACCCCAACACCCTGCACTGGGTCGATGAGGCCGG
GAAGGTCGGAGAGCAGCTGCCGCTGGAGGACCCTGACGTCTACTGCCCCTACAGCGCCATCGGCAACG
TCACGGGAGAGCTGGTGTACGCCCACTACGGGCGGCCCGAAGACCTGCAGGACCTGCGGGCCAGGGGC
GTGGATCCAGTGGGCCGCCTGCTGCTGGTGCGCGTGGGGGTGATCAGCTTCGCCCAGAAGGTGACCAA
TGCTCAGGACTTCGGGGCTCAAGGAGTGCTCATATACCCAGAGCCAGCGGACTTCTCCCAGGACCCAC
CCAAGCCAAGCCTGTCCAGCCAGCAGGCAGTGTATGGACATGTGCACCTGGGAACTGGAGACCCCTAC
ACACCTGGCTTCCCTTCCTTCAATCAAACCCAGTTCCCTCCAGTTGCATCATCAGGCCTTCCCAGCAT
CCCACGCCGAGCCCTATGGAGTCTCAAAAAAAAAAAGGGCCCCGGGCCACGACTGCGGCTAGTGGTCA
ACAATCACAGGACCTCCACCCCCATCAACAACATCTTCGGCTGCATCGAAGGCCGCTCAGAGCCAGAT
CACTACGTTGTCATCGGGGCCCAGAGGGATGCATGGGGCCCAGGAGCAGCTAAATCCGCTGTGGGGAC
GGCTATACTCCTGGAGCTGGTGCGGACCTTTTCCTCCATGGTGAGCAACGGCTTCCGGCCCCGCAGAA
GTCTCCTCTTCATCAGCTGGGACGGTGGTGACTTTGGAAGCGTGGGCTCCACGGAGTGGCNTAGAGGG
CTACCTCAGCGTGCTGCACCTCAAAGCCGTAGTGTACGTGAGCCTGGACAACGCAGTGAAGGGGGATG
ACAAGTTTCATGCCAAGACCAACCCCCTTCTGACAAGTCTCATTGAGAAGTGTCCTGAAGCAGATGGA
TTCTCCCAACCACAGTGGGCAGACTCTCTATGAACAGGTGGGGTTCACCAATTCCCAGCTGGGATTGC
TGAGGTGATCCGGCCCCTACCCATGGGACAGCAGTGCCTATTTCCTTCACGGGCCTTTGTTGGGGAGT
CCCTTGCCCTTCGAGTTCTTCCTTTTATTGGAGGGACGGACCAGGCCTTACCCATTTCCTTGCACAAA
AAGGGAGGGACACTTATTGGAGGAACCTGCAGTAAGGTGCTGCAAGGCCGCCTGCCCGCCGTGGCCCA
GGCCGTGGCCCAGCTCGCAGGGCAGCTCCTCATCCGGCTCAGCCACGATCGCCTGCTGCCCCTCGACT
TCGGCCGCTACGGGGACGTCGTCCTCAGGCACATCGGGAACCTCAACGAGTTCTCTGGGGACCTCAAG
CCCGCGGGCTGACCCTGCAGTGGGTGTACTCGGCGCGGGGGGACTACATCCGGGCGGCGGAAAAGCTG
CGGCAGGTAGATCTACTTGCCGCAGCTCGGAGGAGAGAGACGAGCGACTGACACGCATGTACAACGTC
CGCATAATGCGGGTGGAGTTCTACTTCCTTTCCCAGTACGTGTCGCCAGCCGACTCCCCGTTCCGCCA
CATCTTCATGGGCCGTGGAGACCACACGCTGGGCGCCCTGCTGGACCACCTGCGGCTGCTGCACTCCA
ACAGCTCCGGGACCCCCGGGGCCACCTCCTCCACTGGCTTCCAGGGAGAGCCGTTTCCGGGTCGTCAG
CTAGCCCTGCTCACCTGGACGCTGCAAGGGGCAGCCAATGCGCTTAGCGGGGATGTCTGGAACATTGA
TAACAACTTCTGAGGCCCTGGGGATCCTCAAATCCCCGTCCCCCAGTCAAGAGCTCCTCTGCTCCTCG
CTTGAATGATTCAGGGTCAGGGAGGTGGCTCAGAGTCCACCTCTCATTGCTGATCAATTTCTCATTAC
CCCTACACATCTCTCCACGGAGCCCAGACCCCAGCACAGATATCCACACACCCCAGCCCTGCAGTGTA
GCTGACCCTAATGTGACGGTCATACTGTCGGTTAATCAGAGAGTAGCATCCCTTCAATCACAGCCCCT
TCCCCTTTCTGGGGTCCTCCATACCTAGAGACCACTCTGGGAGGTTTGCTAGGCCCTGGGACCTGGCC
AGCTCTGTTAGTGGGAGAGATCGCTGGCACCATAGCCTTATGGCCAACAGGTGGTCTGTGGTGAAAGG
GGCGTGGAGTTTCAATATCAATAAACCACCTGATATCAATAAAAAAAAAAAAAAAAAAAAAAAAAAAA
A

| | | | |
|---|---|---|---|
| NOV17b, CG52534-01 | SEQ ID NO: 460 | 489 aa | MW at 53555.7kD |
| Protein Sequence | | | |

MERLWGLFQRAQQLSPRSSQTVYQRVEGPRKGHLEEEEEDGEEGAETLAHFCPMELRGPEPLGSRPRQ
PNLIPWAAAGRRAAPYLVLTALLIFTGAFLLGYVAFRGSCQACGDSVLVVSEDVNYEPDLDFHQGRLY
WSDLQAMFLQFLGEGRLEDTIRQTSLRERVAGSAGMAAADSGHSRGLSRQKLDHVWTDTHYVGLQFPD
PAHPNTLHWVDEAGKVGEQLPLEDPDVYCPYSAIGNVTGELVYAHYGRPEDLQDLRARGVDPVGRLLL
VRVGVISFAQKVTNAQDFGAQGVLIYPEPADFSQDPPKPSLSSQQAVYGHVHLGTGDPYTPGFPSFNQ
TQFPPVASSGLPSIPRRALWSLKKKKGPGPRLRLVVNNHRTSTPINNIFGCIEGRSEPDHYVVIGAQR
DAWGPGAAKSAVGTAILLELVRTFSSMVSNGFRPRRSLLFISWDGGDFGSVGSTEWXRGLPQRAAPQS
RSVREPGQRSEGG

| | | |
|---|---|---|
| NOV17c, CG52534-02 | SEQ ID NO: 461 | 865 bp |
| DNA Sequence | ORF Start: ATG at 27 | ORF Stop: TGA at 837 |

TGCGCGCGGATTCTGCTTCTCAGAAGATGCACTATTATAGATACTCTAACGCCAAGGTCAGCTGCTGG
TACAAGTACCTCCTTTTCAGCTACAACATCATCTTCTGGTTGGCTGGAGTTGTCTTCCTTGGAGTCGG
GCTGTGGGCATGGAGCGAAAAGGGTGTGCTGTCCGACCTCACCAAAGTGACCCGGATGCATGGAATCG
ACCCTGTGGTGCTGGTCCTGATGGTGGGCGTGGTGATGTTCACCCTGGGGTTCGCCGGCTGCGTGGGG
GCTCTGCGGGAGAATATCTGCTTGCTCAACTTTTTCTGTGGCACCATCGTGCTCATCTTCTTCCTGGA
GCTGGCTGTGGCCGTGCTGGCCTTCCTGTTCCAGGACTGGGTGAGGGACCGGTTCCGGGAGTTCTTCG
AGAGCAACATCAAGTCCTACCGGGACGATATCGATCTGCAAAACCTCATCGACTCCCTTCAGAAAGCT
AACCAGTGCTGTGGCGCATATGGCCCTGAAGACTGGGACCTCAACGTCTACTTCAATTGCAGCGGTGC
CAGCTACAGCCGAGAGAAGTGCGGGGTCCCCTTCTCCTGCTGCGTGCCAGATCCTGTGCAAAAAGTTG
TGAACACACAGTGTGGATATGATGTCAGGATTCAGCTGAAGAGCAAGTGGGATGAGTCCATCTTCACG
AAAGGCTGCATCCAGGCGCTGGAAAGCTGGCTCCCGCGGAACATTTACATTGTGGCTGGCGTCTTCAT
CGCCATCTCGCTGTTGCAGATATTTGGCATCTTCCTGGCAAGGACGCTGATCTCAGACATCGAGGCAG
TGAAGGCCGGCCATCACTTCTGAGGAGCAGAGTTGAGGGAGCCGAGCTA

| | | | |
|---|---|---|---|
| NOV17c, CG52534-02 | SEQ ID NO: 462 | 270 aa | MW at 30718.5kD |
| Protein Sequence | | | |

MHYYRYSNAKVSCWYKYLLFSYNIIFWLAGVVFLGVGLWAWSEKGVLSDLTKVTRMHGIDPVVLVLMV
GVVMFTLGFAGCVGALRENICLLNFFCGTIVLIFFLELAVAVLAFLFQDWVRDRFREFFESNIKSYRD
DIDLQNLIDSLQKANQCCGAYGPEDWDLNVYFNCSGASYSREKCGVPFSCCVPDPVQKVVNTQCGYDV
RIQLKSKWDESIFTKGCIQALESWLPRNIYIVAGVFIAISLLQIFGIFLARTLISDIEAVKAGHHF

| | | |
|---|---|---|
| NOV17d, CG52534-03 | SEQ ID NO: 463 | 863 bp |
| DNA Sequence | ORF Start: ATG at 26 | ORF Stop: TGA at 836 |

GCGCGCGGATTCTGCTTCTCAGAAGATGCACTATTATAGATACTCTAACGCCAAGGTCAGCTGCTGGT
ACAAGTACCTCCTTTTCAGCTACAACATCATCTTCTGGTTGGCTGGAGTTGTCTTCCTTGGAGTCGGG
CTGTGGGCATGGAGCGAAAAGGGTGTGCTGTCCGACCTCACCAAAGTGACCCGGATGCATGGAATCGA
CCCTGTGGTGCTGGTCCTGATGGTGGGCGTGGTGATGTTCACCCTGGGGTTCGCCGGCTGCGTGGGGG
CTCTGCGGGAGAATATCTGCTTGCTCAACTTTTTCTGTGGCACCATCGTGCTCATCTTCTTCCTGGAG
CTGGCTGTGGCCGTGCTGGCCTTCCTGTTCCAGGACTGGGTGAGGGACCGGTTCCGGGAGTTCTTCGA
GAGCAACATCAAGTCCTACCGGGACGATATCGATCTGCAAAACCTCATCGACTCCCTTCAGAAAGCTA
ACCAGTGCTGTGGCGCATATGGCCCTGAAGACTGGGACCTCAACGTCTACTTCAATTGCAGCGGTGCC
AGCTACAGCCGAGAGAAGTGCGGGGTCCCCTTCTCCTGCTGCGTGCCAGATCCTGCGCAAAAAGTTGT
GAACACACAGTGTGGATATGATGTCAGGATTCAGCTGAAGAGCAAGTGGGATGAGTCCATCTTCACGA
AAGGCTGCATCCAGGCGCTGGAAAGCTGGCTCCCGCGGAACATTTACATTGTGGCTGGCGTCTTCATC
GCCATCTCGCTGTTGCAGATATTTGGCATCTTCCTGGCAAGGACGCTGATCTCAGACATCGAGGCAGT
GAAGGCCGGCCATCACTTCTGAGGAGCAGAGTTGAGGGAGCCGAGCT

| | | | |
|---|---|---|---|
| NOV17d, CG52534-03 | SEQ ID NO: 464 | 270 aa | MW at 30690.5kD |
| Protein Sequence | | | |

MHYYRYSNAKVSCWYKYLLFSYNIIFWLAGVVFLGVGLWAWSEKGVLSDLTKVTRMHGIDPVVLVLMV
GVVMFTLGFAGCVGALRENICLLNFFCGTIVLIFFLELAVAVLAFLFQDWVRDRFREFFESNIKSYRD
DIDLQNLIDSLQKANQCCGAYGPEDWDLNVYFNCSGASYSREKCGVPFSCCVPDPAQKVVNTQCGYDV
RIQLKSKWDESIFTKGCIQALESWLPRNIYIVAGVFIAISLLQIFGIFLARTLISDIEAVKAGHHF

| | | |
|---|---|---|
| NOV17e, CG52534-04 | SEQ ID NO: 465 | 2406 bp |
| DNA Sequence | ORF Start: ATG at 1 | ORF Stop: TGA at 2404 |

ATGGAGCGGCTTTGGGGTCTATTCCAGAGAGCGCAACAACTGTCCCCAAGATCCTCTCAGACCGTCTA
CCAGCGTGTGGAAGGCCCCCGGAAAGGGCACCTGGAGGAGGAAGAGGAAGACGGGGAGGAGGGGGCGG
AGACATTGGCCCACTTCTGCCCCATGGAGCTGAGGGGCCCTGAGCCCCTGGGCTCTAGACCCAGGCAG
CCAAACCTCATTCCCTGGGCGGCAGCAGGACGGAGGGCTGCCCCCTACCTGGTCCTGACGGCCCTGCT
GATCTTCACTGGGGCCTTCCTACTGGGCTACGTCGCCTTCCGAGGGTCCTGCCAGGCGTGCGGAGACT
CTGTGTTGGTGGTCAGTGAGGATGTCAACTATGAGCCTGACCTGGATTTCCACCAGGGCAGACTCTAC
TGGAGCGACCTCCAGGCCATGTTCCTGCAGTTCCTGGGGGAGGGGCGCCTGGAGGACACCATCAGGCA
AACCAGCCTTCGGGAACGGGTGGCAGGCTCGGCCGGGATGGCCGCTCTGACTCAGGACATTCGCGCGG
CGCTCTCCCGCCAGAAGCTGGACCACGTGTGGACCGACACGCACTACGTGGGGCTGCAATTCCCGGAT
CCGGCTCACCCCAACACCCTGCACTGGGTCGATGAGGCCGGGAAGGTCGGAGAGCAGCTGCCGCTGGA
GGACCCTGACGTCTACTGCCCCTACAGCGCCATCGGCAACGTCACGGGAGAGCTGGTGTACGCCCACT
ACGGGCGGCCCGAAGACCTGCAGGACCTGCGGGCCAGGGGCGTGGATCCAGTGGGCCGCCTGCTGCTG
GTGCGCGTGGGGGTGATCAGCTTCGCCCAGAAGGTGACCAATGCTCAGGACTTCGGGGCTCAAGGAGT
GCTCATATACCCAGAGCCAGCGGACTTCTCCCAGGACCCACCCAAGCCAAGCCTGTCCAGCCAGCAGG
CAGTGTATGGACATGTGCACCTGGGAACTGGAGACCCCTACACACCTGGCTTCCCTTCCTTCAATCAA
ACCCAGTTCCCTCCAGTTGCATCATCAGGCCTTCCCAGCATCCCAGCCCAGCCCATCAGTGCAGACAT
TGCCTCCCGCCTGCTGAGGAAGCTCAAAGGCCCTGTGGCCCCCCAAGAATGGCAGGGGAGCCTCCTAG
GCTCCCCTTATCACCTGGGCCCCGGGCCACGACTGCGGCTAGTGGTCAACAATCACAGGACCTCCACC
CCCATCAACAACATCTTCGGCTGCATCGAAGGCCGCTCAGAGCCAGATCACTACGTTGTCATCGGGGC
CCAGAGGGATGCATGGGGCCCAGGAGCAGCTAAATCCGCTGTGGGGACGGCTATACTCCTGGAGCTGG
TGCGGACCTTTTCCTCCATGGTGAGCAACGGCTTCCGGCCCCGCAGAAGTCTCCTCTTCATCAGCTGG
GACGGTGGTGACTTTGGAAGCGTGGGCTCCACGGAGTGGCTAGAAGGCTACCTCAGCGTGCTGCACCT
CAAAGCCGTAGTGTACGTGAGCCTGGACAACGCAGTGCTGGGGGATGACAAGTTTCATGCCAAGACCA
GCCCCCTTCTGACAAGTCTCATTGAGAGTGTCCTGAAGCAGGTGGATTCTCCCAACCACAGTGGGCAG
ACTCTCTATGAACAGGTGGTGTTCACCAATCCCAGCTGGGATGCTGAGGTGATCCGGCCCCTACCCAT
GGACAGCAGTGCCTATTCCTTCACGGCCTTTGTGGGAGTCCCTGCCGTCGAGTTCTCCTTTATGGAGG
ACGACCAGGCCTACCCATTCCTGCACACAAAGGAGGACACTTATGAGAACCTGCATAAGGTGCTGCAA
GGCCGCCTGCCCGCCGTGGCCCAGGCCGTGGCCCAGCTCGCAGGGCAGCTCCTCATCCGGCTCAGCCA
CGATCGCCTGCTGCCCCTCGACTTCGGCCGCTACGGGGACGTCGTCCTCAGGCACATCGGGAACCTCA
ACGAGTTCTCTGGGGACCTCAAGGCCCGCGGGCTGACCCTGCAGTGGGTGTACTCGGCGCGGGGGGAC
TACATCCGGGCGGCGGAAAAGCTGCGGCAGGAGATCTACAGCTCGGAGGAGAGAGACGAGCGACTGAC
ACGCATGTACAACGTGCGCATAATGCGGGTGGAGTTCTACTTCCTTTCCCAGTACGTGTCGCCAGCCG
ACTCCCCGTTCCGCCACATCTTCATGGGCCGTGGAGACCACACGCTGGGCGCCCTGCTGGACCACCTG
CGGCTGCTGCGCTCCAACAGCTCCGGGACCCCCGGGGCCACCTCCTCCACTGGCTTCCAGGAGAGCCG
TTTCCGGCGTCAGCTAGCCCTGCTCACCTGGACGCTGCAAGGGGCAGCCAATGCGCTTAGCGGGGATG
TCTGGAACATTGATAACAACTTCTGA

| | | | |
|---|---|---|---|
| NOV17e, CG52534-04 | SEQ ID NO: 466 | 801 aa | MW at 88754.1kD |
| Protein Sequence | | | |

MERLWGLFQRAQQLSPRSSQTVYQRVEGPRKGHLEEEEEDGEEGAETLAHFCPMELRGPEPLGSRPRQ
PNLIPWAAAGRRAAPYLVLTALLIFTGAFLLGYVAFRGSCQACGDSVLVVSEDVNYEPDLDFHQGRLY
WSDLQAMFLQFLGEGRLEDTIRQTSLRERVAGSAGMAALTQDIPAALSRQKLDHVWTDTHYVGLQFPD
PAHPNTLHWVDEAGKVGEQLPLEDPDVYCPYSAIGNVTGELVYAHYGRPEDLQDLRARGVDPVGRLLL
VRVGVISFAQKVTNAQDFGAQGVLIYPEPADFSQDPPKPSLSSQQAVYGHVHLGTGDPYTPGFPSFNQ
TQFPPVASSGLPSIPAQPISADIASRLLRKLKGPVAPQEWQGSLLGSPYHLGPGPRLRLVVNNHRTST
PINNIFGCIEGRSEPDHYVVIGAQRDAWGPGAAKSAVGTAILLELVRTFSSMVSNGFRPRRSLLFISW
DGGDFGSVGSTEWLEGYLSVLHLKAVVYVSLDNAVLGDDKFHAKTSPLLTSLIESVLKQVDSPNHSGQ
TLYEQVVFTNPSWDAEVIRPLPMDSSAYSFTAFVGVPAVEFSFMEDDQAYPFLHTKEDTYENLHKVLQ
GRLPAVAQAVAQLAGQLLIRLSHDRLLPLDFGRYGDVVLRHIGNLNEFSGDLKARGLTLQWVYSARGD
YIRAAEKLRQEIYSSEERDERLTRMYNVRIMRVEFYFLSQYVSPADSPFRHIFMGRGDHTLGALLDHL
RLLRSNSSGTPGATSSTGFQESRFRRQLALLTWTLQGAANALSGDVWNIDNNF

| | | |
|---|---|---|
| NOV17f, CG52534-05 | SEQ ID NO: 467 | 2531 bp |
| DNA Sequence | ORF Start: ATG at 65 | ORF Stop: TGA at 2468 |

CTGCAGGCTTCAGGAGGGGTACTCTGCCTTCTGGACACTGCAGGCTTCAGGAGGGGACACAAGCATGG
AGCGGCTTTGGGGTCTATTCCAGAGAGCGCAACAACTGTCCCCAAGATCCTCTCAGACCGTCTACCAG
CGTGTGGAAGGCCCCCGGAAAGGGCACCTGGAGGAGGAAGAGGAAGACGGGGAGGAGGGGGCGGAGAC
ATTGGCCCACTTCTGCCCCATGGAGCTGAGGGGCCCTGAGCCCCTGGGCTCTAGACCCAGGCAGCCAA
ACCTCATTCCCTGGGCGGCAGCAGGACGGAGGGCTGCCCCCTACCTGGTCCTGACGGCCCTGCTGATC
TTCACTGGGGCCTTCCTACTGGGCTACGTCGCCTTCCGAGGGTCCTGCCAGGCGTGCGGAGACTCTGT
GTTGGTGGTCAGTGAGGATGTCAACTATGAGCCTGACCTGGATTTCCACCAGGGCAGACTCTACTGGA
GCGACCTCCAGGCCATGTTCCTGCAGTTCCTGGGGGAGGGGCGCCTGGAGGACACCATCAGGCAAACC
AGCCTTCGGGAACGGGTGGCAGGCTCGGCCGGGATGGCCGCTCTGACTCAGGACATTCGCGCGGCGCT
CTCCTGCCAGAAGCTGGACCACGTGTGGACCGACACGCACTACGTGGGGCTGCAATTCCCGGATCCGG
CTCACCCCAACACCCTGCACTGGGTCGATGAGGCCGGGAAGGTCGGAGAGCAGCTGCCGCTGGAGGAC
CCTGACGTCTACTGCCCCTACAGCGCCATCGGCAACGTCACGGGAGAGCTGGTGTACGCCCACTACGG
GCGGCCCGAAGACTTGCAGGACCTGCGGGCCAGGGGCGTGGATCCAGTGGGCCGCCTGCTGCTGGTGC
GCGTGGGGGTGATCAGCTTCGCCCAGAAGGTGACCAATGCTCAGGACTTCGGGGCTCAAGGAGTGCTC
ATATACCCAGAGCCAGCGGACTTCTCCCAGGACCCACCCAAGCCAAGCCTGTCCAGCCAGCAGGCAGT
GTATGGACATGTGCACCTGGGAACTGGGGACCCCTACACACCTGGCTTCCCTTCCTTCAATCAAACCC
AGTTCCCTCCAGTTGCATCATCAGGCCTTCCCAGCATCCCAGCCCAGCCCATCAGTGCAGACATTGCC
TCCCGCCTGCTGAGGAAGCTCAAAGGCCCTGTGGCCCCCCAAGAATGGCAGGGGAGCCTCCTAGGCTC
CCCTTATCACCTGGGCCCCGGGCCACGACTGCGGCTAGTGGTCAACAATCACAGGACCTCCACCCCCA
TCAACAACATCTTCGGCTGCATCGAAGGCCGCTCAGAGCCAGATCACTACGTTGTCATCGGGGCCCAG
AGGGATGCATGGGGCCCAGGAGCAGCTAAATCCGCTGTGGGGACGGCTATACTCCTGGAGCTGGTGCG
GACCTTTTCCTCCATGGTGAGCAACGGCTTCCGGCCCCGCAGAAGTCTCCTCTTCATCAGCTGGGACG
GTGGTGACTTTGGAAGCGTGGGCTCCACGGAGTGGCTAGAGGGCTACCTCAGCGTGCTGCACCTCAAA
GCCGTAGTGTACGTGAGCCTGGACAACGCAGTGCTGGGGGATGACAAGTTTCATGCCAAGACCAGCCC
CCTTCTGACAAGTCTCATTGAGAGTGTCCTGAAGCAGGTGGATTCTCCCAACCACAGTGGGCAGACTC
TCTATGAACAGGTGGTGTTCACCAATCCCAGCTGGGATGCTGAGGTGATCCGGCCCCTACCCATGGAC
AGCAGTGCCTATTCCTTCACGGCCTTTGTGGGAGTCCCTGCCGTCGAGTTCTCCTTTATGGAGGACGA
CCAGGCCTACCCATTCCTGCACACAAAGGAGGACACTTATGAGAACCTGCATAAGGTGCTGCAAGGCC
GCCTGCCCGCTGTGGCCCAGGCCGTGGCCCAGCCCGCAGGGCAGCTCCTCATCCGGCTCAGCCACGAT
CGCCTGCTGCCCCTCGACTTCGGCCGCTACGGGGACGTCGTCCTCAGGCACATCGGGAACCTCAACGA
GTTCTCTGGGGACCTCAAGGCCCGCGGGCTGACCCTGCAGTGGGTGTACTCGGCGCGGGGGGACTACA
TCCGGGCGGCGGAAAAGCTGCGGCAGGAGATCTACAGCTCGGAGGAGAGAGACGAGCGACTGACACGC
ATGTACAACGTGCGCATAATGCGGGTGGAGTTCTACTTCCTTTCCCAGTACGTGTCGCCAGCCGACTC
CCCGTTCCGCCACATCTTCATGGGCCGTGGAGACCACACGCTGGGCGCCCTGCTGGACCACCTGCGGC
TGCTGCGCTCCAACAGCTCCGGGACCCCCGGGGCCACCTCCTCCACTGGCTTCCAGGAGAGCCGTTTC
CGGCGTCAGCTAGCCCTGCTCACCTGGACGCTGCAAGGGGCAGCCAATGCGCTTAGCGGGGATGTCTG
GAACATTGATAACAACTTCTGAGGCCCTGGGGATCCTCACATCCCCGTCCCCCAGTCAAGAGCTCCTC
TGCACCTCGACTTGA

| | | | |
|---|---|---|---|
| NOV17f, CG52534-05 | SEQ ID NO: 468 | 801 aa | MW at 88685.0kD |
| Protein Sequence | | | |

MERLWGLFQRAQQLSPRSSQTVYQRVEGPRKGHLEEEEEDGEEGAETLAHFCPMELRGPEPLGSRPRQ
PNLIPWAAAGRRAAPYLVLTALLIFTGAFLLGYVAFRGSCQACGDSVLVVSEDVNYEPDLDFHQGRLY
WSDLQAMFLQFLGEGRLEDTIRQTSLRERVAGSAGMAALTQDIRAALSCQKLDHVWTDTHYVGLQFPD
PAHPNTLHWVDEAGKVGEQLPLEDPDVYCPYSAIGNVTGELVYAHYGRPEDLQDLRARGVDPVGRLLL
VRVGVISFAQKVTNAQDFGAQGVLIYPEPADFSQDPPKPSLSSQQAVYGHVHLGTGDPYTPGFPSFNQ
TQFPPVASSGLPSIPAQPISADIASRLLRKLKGPVAPQEWQGSLLGSPYHLGPGPRLRLVVNNHRTST
PINNIFGCIEGRSEPDHYVVIGAQRDAWGPGAAKSAVGTAILLELVRTFSSMVSNGFRPRRSLLFISW
DGGDFGSVGSTEWLEGYLSVLHLKAVVYVSLDNAVLGDDKFHAKTSPLLTSLIESVLKQVDSPNHSGQ
TLYEQVVFTNPSWDAEVIRPLPMDSSAYSFTAFVGVPAVEFSFMEDDQAYPFLHTKEDTYENLHKVLQ
GRLPAVAQAVAQPAGQLLIRLSHDRLLPLDFGRYGDVVLRHIGNLNEFSGDLKARGLTLQWVYSARGD
YIRAAEKLRQEIYSSEERDEPLTRMYNVRIMRVEFYFLSQYVSPADSPFRHIFMGRGDHTLGALLDHL
RLLRSNSSGTPGATSSTGFQESRFRRQLALLTWTLQGAANALSGDVWNIDNNF

A ClustalW comparison of the above protein sequences yields the following sequence alignment shown in Table 17B.

TABLE 17B
Comparison of the NOV17 protein sequences.

| | |
|---|---|
| NOV17a | MERLWGLFQRAQQLSPRSSQTVYQRVEGPRKGHLEEEEEDGEEGAETLAHFCPMELRGPE |
| NOV17b | MERLWGLFQRAQQLSPRSSQTVYQRVEGPRKGHLEEEEEDGEEGAETLAHFCPMELRGPE |
| NOV17c | --------MHYYRYSNAKVSCWYKYLLFSYNIIFWLA---G-------VVFLGVGLWAWS |
| NOV17d | --------MHYYRYSNAKVSCWYKYLLFSYNIIFWLA---G-------VVFLGVGLWAWS |
| NOV17e | MERLWGLFQRAQQLSPRSSQTVYQRVEGPRKGHLEEEEEDGEEGAETLAHFCPMELRGPE |
| NOV17f | MERLWGLFQRAQQLSPRSSQTVYQRVEGPRKGHLEEEEEDGEEGAETLAHFCPMELRGPE |
| NOV17a | PLGSRPRQPNLIPWAAAGRRAAPYLVLTALLIFTGAFLLGYVAFRG-SCQA---CGDSVL |
| NOV17b | PLGSRPRQPNLIPWAAAGRRAAPYLVLTALLIFTGAFLLGYVAFRG-SCQA---CGDSVL |
| NOV17c | EKGVLSDLTKVT--RMHGIDPVVLVLMVGVVMFTLGFAGCVGALRENICLLNFFCGTIVL |
| NOV17d | EKGVLSDLTKVT--RMHGIDPVVLVLMVGVVMFTLGFAGCVGALRENICLLNFFCGTIVL |
| NOV17e | PLGSRPRQPNLIPWAAAGRRAAPYLVLTALLIFTGAFLLGYVAFRG-SCQA---CGDSVL |
| NOV17f | PLGSRPRQPNLIPWAAAGRRAAPYLVLTALLIFTGAFLLGYVAFRG-SCQA---CGDSVL |
| NOV17a | VVSEDVNYEPDLDFHQGRLYWSDLQAMFLQFLGEGRLEDTIRQTSLRERVAGSAGMAALT |
| NOV17b | VVSEDVNYEPDLDFHQGRLYWSDLQAMFLQFLGEGRLEDTIRQTSLRERVAGSAGMAAAD |
| NOV17c | IFFLELAVA-VLAFLF--QDW--VRDRFREFF-----ESNIK--SYRDDIDLQNLIDSLQ |
| NOV17d | IFFLELAVA-VLAFLF--QDW--VRDRFREFF-----ESNIK--SYRDDIDLQNLIDSLQ |
| NOV17e | VVSEDVNYEPDLDFHQGRLYWSDLQAMFLQFLGEGRLEDTIRQTSLRERVAGSAGMAALT |
| NOV17f | VVSEDVNYEPDLDFHQGRLYWSDLQAMFLQFLGEGRLEDTIRQTSLRERVAGSAGMAALT |
| NOV17a | QDIRAALSRQKLDHVWTDTHYVGLQFPDPAHPNTLHWVDEAGKVGEQLPLEDPDVYCPYS |
| NOV17b | SGHSRGLSRQKLDHVWTDTHYVGLQFPDPAHPNTLHWVDEAGKVGEQLPLEDPDVYCPYS |
| NOV17c | KANQCCGAYGPED--WDLNVYFNCSGASYSREKCGVPFSCCVPDPVQKVV---NTQCGYD |
| NOV17d | KANQCCGAYGPED--WDLNVYFNCSGASYSREKCGVPFSCCVPDPAQKVV---NTQCGYD |
| NOV17e | QDIRAALSRQKLDHVWTDTHYVGLQFPDPAHPNTLHWVDEAGKVGEQLPLEDPDVYCPYS |
| NOV17f | QDIRAALSCQKLDHVWTDTHYVGLQFPDPAHPNTLHWVDEAGKVGEQLPLEDPDVYCPYS |
| NOV17a | AIGNVTGELVYAHYGRPEDLQDLRARGVDPVGRLLLVRVGVISFAQKVTNAQDFGAQGVL |
| NOV17b | AIGNVTGELVYAHYGRPEDLQDLRARGVDPVGRLLLVRVGVISFAQKVTNAQDFGAQGVL |
| NOV17c | VRIQLKSKWDESIFTKGC-IQALESW--LP--RNIYIVAGV--FIA-ISLLQIFG----- |
| NOV17d | VRIQLKSKWDESIFTKGC-IQALESW--LP--RNIYIVAGV--FIA-ISLLQIFG----- |
| NOV17e | AIGNVTGELVYAHYGRPEDLQDLRARGVDPVGRLLLVRVGVISFAQKVTNAQDFGAQGVL |
| NOV17f | AIGNVTGELVYAHYGRPEDLQDLRARGVDPVGRLLLVRVGVISFAQKVTNAQDFGAQGVL |
| NOV17a | IYPEPADFSQDPPKPSLSSQQAVYGHVHLGTGDPYTPGFPSFNQTQFPPVASSGLPSIPA |
| NOV17b | IYPEPADFSQDPPKPSLSSQQAVYGHVHLGTGDPYTPGFPSFNQTQFPPVASSGLPSIPR |
| NOV17c | IFLARTLIS-D--IEAVK--A---GH-HF------------------------------- |
| NOV17d | IFLARTLIS-D--IEAVK--A---GH-HF------------------------------- |
| NOV17e | IYPEPADFSQDPPKPSLSSQQAVYGHVHLGTGDPYTPGFPSFNQTQFPPVASSGLPSIPA |
| NOV17f | IYPEPADFSQDPPKPSLSSQQAVYGHVHLGTGDPYTPGFPSFNQTQFPPVASSGLPSIPA |
| NOV17a | QPISADIASRLLDHLRLLRSNSSGTPGATSSTGFQESRFRRQLALLTWTLQGAANALSGD |
| NOV17b | R------ALWSLKKKKGP---------------G----PRLRLVVNNHRTSTPINNIFGC |
| NOV17c | ------------------------------------------------------------ |
| NOV17d | ------------------------------------------------------------ |
| NOV17e | QPISADIASRLLRKLKGPVAPQEWQGSLLGSPYHLGPGPRLRLVVNNHRTSTPINNIFGC |
| NOV17f | QPISADIASRLLRKLKGPVAPQEWQGSLLGSPYHLGPGPRLRLVVNNHRTSTPINNIFGC |
| NOV17a | VWNIDNNF---------------------------------------------------- |
| NOV17b | IEGRSEPDHYVVIGAQRDAWGPGAAKSAVGTAILLELVRTFSSMVSNGFRPRRSLLFISW |
| NOV17c | ------------------------------------------------------------ |
| NOV17d | ------------------------------------------------------------ |
| NOV17e | IEGRSEPDHYVVIGAQRDAWGPGAAXSAVGTAILLELVRTFSSMVSNGFRPRRSLLFISW |
| NOV17f | IEGRSEPDHYVVIGAQRDAWGPGAAKSAVGTAILLELVRTFSSMVSNGFRPRRSLLFISW |
| NOV17a | ------------------------------------------------------------ |
| NOV17b | DGGDFGSVGSTEWXRGLPQRAAPQSRSVREPGQRSEGG---------------------- |
| NOV17c | ------------------------------------------------------------ |
| NOV17d | ------------------------------------------------------------ |
| NOV17e | DGGDFGSVGSTEWLEGYLSVLHLKAVVYVSLDNAVLGDDKFHAKTSPLLTSLIESVLKQV |
| NOV17f | DGGDFGSVGSTEWLEGYLSVLHLKAVVYVSLDNAVLGDDKFHAKTSPLLTSLIESVLKQV |
| NOV17a | ------------------------------------------------------------ |
| NOV17b | ------------------------------------------------------------ |
| NOV17c | ------------------------------------------------------------ |
| NOV17d | ------------------------------------------------------------ |
| NOV17e | DSPNHSGQTLYEQVVFTNPSWDAEVIRPLPMDSSAYSFTAFVGVPAVEFSFMEDDQAYPF |
| NOV17f | DSPNHSGQTLYEQVVFTNPSWDAEVIRPLPMDSSAYSFTAFVGVPAVEFSFMEDDQAYPF |
| NOV17a | ------------------------------------------------------------ |
| NOV17b | ------------------------------------------------------------ |
| NOV17c | ------------------------------------------------------------ |
| NOV17d | ------------------------------------------------------------ |
| NOV17e | LHTKEDTYENLHKVLQGRLPAVAQAVAQLAGQLLIRLSHDRLLPLDFGRYGDVVLRHIGN |
| NOV17f | LHTKEDTYENLHKVLQGRLPAVAQAVAQPAGQLLIRLSHDRLLPLDFGRYGDVVLRHIGN |
| NOV17a | ------------------------------------------------------------ |
| NOV17b | ------------------------------------------------------------ |
| NOV17c | ------------------------------------------------------------ |
| NOV17d | ------------------------------------------------------------ |
| NOV17e | LNEFSGDLKARGLTLQWVYSARGDYIRAAEKLRQEIYSSEERDERLTRMYNVRIMRVEFY |
| NOV17f | LNEFSGDLKARGLTLQWVYSARGDYIRAAEKLRQEIYSSEERDERLTRMYNVRIMRVEFY |
| NOV17a | ------------------------------------------------------------ |
| NOV17b | ------------------------------------------------------------ |
| NOV17c | ------------------------------------------------------------ |
| NOV17d | ------------------------------------------------------------ |
| NOV17e | FLSQYVSPADSPFRHIFMGRGDHTLGALLDHLRLLRSNSSGTPGATSSTGFQESRFRRQL |
| NOV17f | FLSQYVSPADSPFRHIFMGRGDHTLGALLDHLRLLRSNSSGTPGATSSTGFQESRFRRQL |
| NOV17a | ------------------------- |
| NOV17b | ------------------------- |
| NOV17c | ------------------------- |
| NOV17d | ------------------------- |
| NOV17e | ALLTWTLQGAANALSGDVWNIDNNF |
| NOV17f | ALLTWTLQGAANALSGDVWNIDNNF |
| NOV17a | (SEQ ID NO: 458) |
| NOV17b | (SEQ ID NO: 460) |
| NOV17c | (SEQ ID NO: 462) |
| NOV17d | (SEQ ID NO: 464) |
| NOV17e | (SEQ ID NO: 466) |
| NOV17f | (SEQ ID NO: 468) |

Further analysis of the NOV17a protein yielded the following properties shown in Table 17C.

TABLE 17C
Protein Sequence Properties NOV17a

| | |
|---|---|
| SignalP analysis: | No Known Signal Sequence Predicted |
| PSORT II analysis: | |

| PSG: | a new signal peptide prediction method | |
|---|---|---|
| | N-region: | length 10; pos. chg 2; neg. chg 1 |
| | H-region: | length 6; peak value −7.91 |
| | PSG score: | −12.31 |

| | |
|---|---|
| GvH: | von Heijne's method for signal seq. recognition |
| | GvH score (threshold: −2.1): −6.46 |
| | possible cleavage site: between 16 and 17 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 1 | | |
| | Number of TMS(s) for threshold 0.5: 1 | | |
| | INTEGRAL | Likelihood = | Transmembrane 86-102 |
| | | −5.68 | |

| | |
|---|---|
| PERIPHERAL | Likelihood = 1.54 (at 264) |

| | |
|---|---|
| | ALOM score: −5.68 (number of TMSs: 1) |
| MTOP: | Prediction of membrane topology (Hartmann et al.) |
| | Center position for calculation: 93 |
| | Charge difference: −3.0 C(0.0)-N(3.0) |
| | N >= C: N-terminal side will be inside |

>>> membrane topology: type 2 (cytoplasmic tail 1 to 86)

| MITDISC: | discrimination of mitochondrial targeting seq | | | |
|---|---|---|---|---|
| | R content: | 4 | Hyd Moment(75): | 8.36 |
| | Hyd Moment(95): | 11.20 | G content: | 1 |
| | D/E content: | 2 | S/T content: | 4 |

| | |
|---|---|
| | Score: −1.76 |
| Gavel: | prediction of cleavage sites for mitochondrial preseq |
| | R-2 motif at 35 QRV\|EG |

NUCDISC: discrimination of nuclear localization signals

| | |
|---|---|
| | pat4: none |
| | pat7: none |
| | bipartite: none |
| | content of basic residues: 8.0% |
| | NLS Score: −0.47 |
| KDEL: | ER retention motif in the C-terminus: none |

ER Membrane Retention Signals:

XXRR-like motif in the N-terminus: ERLW

none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 89 |
| COIL: | Lupas's algorithm to detect coiled-coil regions |
| | total: 0 residues |

Final Results (k = 9/23):

```
47.8%: mitochondrial
26.1%: cytoplasmic
  8.7%: Golgi
  4.3%: vacuolar
  4.3%: vesicles of secretory system
  4.3%: nuclear
  4.3%: endoplasmic reticulum
```

>> prediction for CG52534-06 is mit (k = 23)

A search of the NOV17a protein against the Geneseq database, a proprietary database that contains sequences published in patents and patent publication, yielded several homologous proteins shown in Table 17D.

TABLE 17D
Geneseq Results for NOV17a

| | | NOV17a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAY96211 | Transferrin receptor-like (TfR2) protein - | 1 . . . 372 | 369/372 (99%) | 0.0 |
| | Homo sapiens, 801 aa. | 1 . . . 372 | 370/372 (99%) | |
| | [WO200027874-A2, 18-MAY-2000] | | | |
| ABB11600 | Human transferrin receptor homologue, | 1 . . . 372 | 366/372 (98%) | 0.0 |
| | SEQ ID NO: 1970 - Homo sapiens, 802 | 1 . . . 372 | 367/372 (98%) | |
| | aa. [WO200157188-A2, 09-AUG-2001] | | | |
| AAB93873 | Human protein sequence SEQ ID | 1 . . . 158 | 158/158 (100%) | 9e−90 |
| | NO: 13773 - Homo sapiens, 158 aa. | 1 . . . 158 | 158/158 (100%) | |
| | [EP1074617-A2, 07-FEB-2001] | | | |
| ABP56714 | Human CD71 protein SEQ ID NO: 28 - | 84 . . . 371 | 112/299 (37%) | 1e−42 |
| | Homo sapiens, 760 aa. | 68 . . . 349 | 159/299 (52%) | |
| | [WO2002100336-A2, 19-DEC-2002] | | | |
| ABU04022 | Human expressed protein tag (EPT) | 84 . . . 371 | 112/299 (37%) | 1e−42 |
| | #688 - Homo sapiens, 760 aa. | 68 . . . 349 | 159/299 (52%) | |
| | [WO200278524-A2, 10-OCT-2002] | | | |

In a BLAST search of public sequence databases, the NOV17a protein was found to have homology to the proteins shown in the BLASTP data in Table 17E.

TABLE 17E
Public BLASTP Results for NOV17a

| | | NOV17a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9UP52 | Transferrin receptor protein 2 (TfR2) - | 1 . . . 372 | 369/372 (99%) | 0.0 |
| | Homo sapiens (Human), 801 aa. | 1 . . . 372 | 370/372 (99%) | |
| Q9JKX3 | Transferrin receptor protein 2 (TfR2) - | 1 . . . 371 | 293/371 (78%) | e−166 |
| | Mus musculus (Mouse), 798 aa. | 1 . . . 366 | 313/371 (83%) | |
| Q07891 | Transferrin receptor protein 1 (TfR1) | 87 . . . 371 | 112/296 (37%) | 6e−45 |
| | (TR) (TfR) (Trfr) - Cricetulus griseus | 71 . . . 346 | 160/296 (53%) | |
| | (Chinese hamster), 757 aa. | | | |
| P02786 | Transferrin receptor protein 1 (TfR1) | 84 . . . 371 | 112/299 (37%) | 3e−42 |
| | (TR) (TfR) (Trfr) (CD71 antigen) (T9) | 68 . . . 349 | 159/299 (52%) | |
| | (p90) - Homo sapiens (Human), 760 aa. | | | |
| Q8C872 | Transferrin receptor - Mus musculus | 84 . . . 367 | 107/292 (36%) | 4e−42 |
| | (Mouse), 515 aa. | 68 . . . 347 | 152/292 (51%) | |

PFam analysis predicts that the NOV17a protein contains the domains shown in the Table 17F.

Table 17F. Domain Analysis of NOV17a

TABLE 17F
Domain Analysis of NOV17a

| | | Identities/ | |
|---|---|---|---|
| | | Similarities | |
| Pfam | NOV17a | for the | Expect |
| Domain | Match Region | Matched Region | Value |
| PA | 236 . . . 335 | 36/112 (32%) | 8.7e−14 |
| | | 70/112 (62%) | |
| TFR_dimer | 247 . . . 416 | 34/188 (18%) | 0.45 |
| | | 121/188 (64%) | |

### Example 18

The NOV18 clone was analyzed, and the nucleotide and encoded polypeptide sequences are shown in Table 18A.

TABLE 18A
NOV18 Sequence Analysis

| | | |
|---|---|---|
| NOV18a, CG52979-03 | SEQ ID NO: 469 | 458 bp |

| | | |
|---|---|---|
| DNA Sequence | ORF Start: ATG at 61 | Stop: TAG at 343 |

TCTTCATTCTTTCCGCCATCTTGATTCTTTCTCACTGACCAAGACTCAGCCGTGGGAAATATGAGTGA
GCTTGTAAGAACAACATCCCAATCCTCAGAAAGAGGAAATGACCAAGAGTCTTCCCAGCCAGTTGGAT
CTGTGATTGTCCAGGAGCCCACTGAGGAAAAGCGTCAAGAAGAGGAACCACCAACTGATAATCAGGGG
TGATGTCAGGGAGGGTATTATGCCCACTTTTGATCTCACTAAAGTGCTGGAAGCAGGTGATGCGCAAC
CATAGGTTTCAAGCAAGACAAATGAAGACTGAAACCAAGAACGTTATTCTTAATCTGGAAATTTGACT
GATAATATTCTCTTAATAAAGTTTTAAGTTTTCTGCAAAGAATCCTTAAA

NOV18a, CG52979-03

| | | | |
|---|---|---|---|
| Protein Sequence | SEQ ID NO: 470 | 94 aa | MW at 10341.1kD |

MSELVRTTSQSSERGNDQESSQPVGSVIVQEPTEEKRQEEEPPTDNQGPDMEAFQQELALLKIEDEPG
DGPDVREGIMPTFDLTKVLEAGDAQP

| | | |
|---|---|---|
| NOV18b, CG52979-01 | SEQ ID NO: 471 | 458 bp |

| | | |
|---|---|---|
| DNA Sequence | ORF Start: ATG at 61 | ORF Stop: TAG at 343 |

TCTTCATTCTTTCCGCCATCTTGATTCTTTCTCACTGACCAAAGACTCAGCCGTGGGAAAATATGTGA
GCTTGTAAGAGCAAGATCCCAATCCTCAGAAAGAGGAAATGACCAAGAGTCTTCCCAGCCGGTTGGAT
CTGTGATTGTCCAGGAGCCCACTGAGGAAAAACGTCAAGAAGAGGAACCACCAACTGATAATCAGGGG
CCTGACATGGAAGCTTTTCAACAGGAACTGGCTCTGCTTAAGATAGAGGATGAGCCTGGAGATGGTCC
TGATGTCAGGGAGGGTATTATGCCCACTTTTGATCTCACTAAAGTGCTGGAAGCAGGTGATGCGCAAC
CATAGGTTTCAAGCAAGACAAATGAAGACTGAAACCAAGAACGTTATTCTTAATCTGGAAATTTGACT
GATAATATTCTCTTAATAAAGTTTTAAGTTTTCTGCAAAGAATCCTTAAA

NOV18b, CG52979-01

| | | | |
|---|---|---|---|
| Protein Sequence | SEQ ID NO: 472 | 94 aa | MW at 10366.1kD |

MSELVRTTSQSSERGNDQESSQPVGSVIVQEPTEEKRQEEEPPTDNQGPDMEAFQQELALLKIEDEPG
DGPDVREGIMPTFDLTKVLEAGDAQP

| | | |
|---|---|---|
| NOV18c, CG52979-02 | SEQ ID NO: 473 | 336 bp |

| | | |
|---|---|---|
| DNA Sequence | ORF Start: at 1 | ORF Stop: at 112 |

TGGTTGCGCATCACCTGCTTCCAGCACTTTAGTGAGATCAAAAGTGGGCATAATACCCTCCCTGACAT
CAGGACCATCTCCAGGCTCATCCTCTATCTTAAGCAGAGCCAGTTCCTGTTGAAAAGCTTCCATGTCA
GGCCCTTGAAAAGCAGGCACTGCTTGATTTTCAATCTCCCCACTAGGTGCAATACCCTGATTATCAGT
TGGTGGTTCCTCTTCTTGACGTTTTTCCTCAGTGGGCTCCTGGACAATCACAGATCCAACCGGCTGGG
AAGACTCTTGGTCATTTCCTCTTTCTGAGGATTGGGATCTTGCTCTTACAAGCTCACTCATGGT

NOV18c, CG52979-02

| | | | |
|---|---|---|---|
| Protein Sequence | SEQ ID NO: 474 | 37 aa | MW at 4444.1kD |

WLRITCFQHFSEIKSGHNTLPDIRTISRLILYLKQSQ

A ClustalW comparison of the above protein sequences yields the following sequence alignment shown in Table 18B.

TABLE 18B
Comparison of the NOV18 protein sequences.

| | |
|---|---|
| NOV18a | MSELVRTTSQSSERGNDQESSQPVGSVIVQEPTEEKRQEEEPPTDNQGPDMEAFQQELAL |
| NOV18b | MSELVRARSQSSERGNDQESSQPVGSVIVQEPTEEKRQEEEPPTDNQGPDMEAFQQELAL |
| NOV18c | ----------------------------WLRITCFQHFSEIKSGHNTLPDIRTISRLILY |
| NOV18a | LKIEDEPGDGPDVREGIMPTFDLTKVLEAGDAQP |
| NOV18b | LKIEDEPGDGPDVREGIMPTFDLTKVLEAGDAQP |
| NOV18c | LKQSQ----------------------------- |
| NOV18a (SEQ ID NO: 470) | |
| NOV18b (SEQ ID NO: 472) | |
| NOV18c (SEQ ID NO: 474) | |

Further analysis of the NOV18a protein yielded the following properties shown in Table 18C.

TABLE 18C
Protein Sequence Properties NOV18a

| | |
|---|---|
| SignalP analysis: | No Known Signal Sequence Predicted |
| PSORT II analysis: | |

| PSG: | a new signal peptide prediction method | |
|---|---|---|
| | N-region: | length 6; pos. chg 1; neg. chg 1 |
| | H-region: | length 6; peak value −10.69 |
| | PSG score: | −15.09 |

| | |
|---|---|
| GvH: | von Heijne's method for signal seq. recognition |
| | GvH score (threshold: −2.1): −14.03 |
| | possible cleavage site: between 14 and 15 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 0 | |
| | number of TMS(s) . . . fixed | |
| | PERIPHERAL | Likelihood = 10.87 (at 76) |

| | ALOM score: 10.87 (number of TMSs: 0) | | | |
|---|---|---|---|---|
| MITDISC: | discrimination of mitochondrial targeting seq | | | |
| | R content: | 1 | Hyd Moment(75): | 5.19 |
| | Hyd Moment(95): | 12.09 | G content: | 0 |
| | D/E content: | 2 | S/T content: | 6 |

| | |
|---|---|
| | Score: −3.75 |
| Gavel: | prediction of cleavage sites for mitochondrial preseq |
| | cleavage site motif not found |

NUCDISC: discrimination of nuclear localization signals

| | |
|---|---|
| | pat4: none |
| | pat7: none |
| | bipartite: none |
| | content of basic residues: 7.4% |
| | NLS Score: −0.47 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 94.1 |
| COIL: | Lupas's algorithm to detect coiled-coil regions |
| | total: 0 residues |

Final Results (k = 9/23):

78.3%: nuclear
21.7%: mitochondrial

>> prediction for CG52979-03 is nuc (k = 23)

A search of the NOV18a protein against the Geneseq database, a proprietary database that contains sequences published in patents and patent publication, yielded several homologous proteins shown in Table 18D.

TABLE 18D
Geneseq Results for NOV18a

| | | NOV18a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE08580 | Human NOV1 protein - Homo sapiens, | 1 . . . 94 | 92/94 (97%) | 1e−47 |
| | 94 aa. [WO200161009-A2, 23-AUG- | 1 . . . 94 | 92/94 (97%) | |
| | 2001] | | | |
| AAE08593 | Protein encoded by pCR2.1-83350421- | 1 . . . 94 | 92/111 (82%) | 2e−44 |
| | S747-3A insert - Homo sapiens, 111 aa. | 1 . . . 111 | 92/111 (82%) | |
| | [WO200161009-A2, 23-AUG-2001] | | | |
| AAE08582 | Human NOV3 protein - Homo sapiens, | 1 . . . 94 | 92/111 (82%) | 2e−44 |
| | 111 aa. [WO200161009-A2, 23-AUG- | 1 . . . 111 | 92/111 (82%) | |
| | 2001] | | | |
| ABJ19255 | Human cancer/testis antigen - SEQ ID | 1 . . . 94 | 92/111 (82%) | 4e−44 |
| | No 37 - Homo sapiens, 111 aa. | 1 . . . 111 | 92/111 (82%) | |
| | [WO200278526-A2, 10-OCT-2002] | | | |
| AAE08581 | Human NOV2 protein - Homo sapiens, | 1 . . . 94 | 92/111 (82%) | 4e−44 |
| | 111 aa. [WO200161009-A2, 23-AUG- | 1 . . . 111 | 92/111 (82%) | |
| | 2001] | | | |

In a BLAST search of public sequence databases, the NOV18a protein was found to have homology to the proteins shown in the BLASTP data in Table 18E.

TABLE 18E
Public BLASTP Results for NOV18a

| | | NOV18a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8WWL9 | Page-5 protein - Homo sapiens | 1 . . . 93 | 80/110 (72%) | 4e−35 |
| | (Human), 130 aa. | 21 . . . 129 | 84/110 (75%) | |
| Q96GU1 | Hypothetical protein - Homo sapiens | 1 . . . 93 | 80/110 (72%) | 4e−35 |
| | (Human), 110 aa. | 1 . . . 109 | 84/110 (75%) | |
| O60829 | G antigen family C 1 protein (Prostate- | 6 . . . 94 | 39/104 (37%) | 1e−06 |
| | associated gene protein 4) (PAGE-4) | 4 . . . 102 | 47/104 (44%) | |
| | (PAGE-1) (JM27) (GAGE-9) - Homo | | | |
| | sapiens (Human), 102 aa. | | | |
| Q8WTP9 | Placenta-specific gene 6 protein (XAGE- | 31 . . . 94 | 31/82 (37%) | 4e−06 |
| | 3 protein) (pp9012) - Homo sapiens | 28 . . . 109 | 43/82 (51%) | |
| | (Human), 111 aa. | | | |
| Q8WWM1 | XAGE-5 protein - Homo sapiens | 21 . . . 94 | 32/92 (34%) | 7e−06 |
| | (Human), 108 aa. | 17 . . . 106 | 49/92 (52%) | |

PFam analysis predicts that the NOV18a protein contains the domains shown in the Table 18F.

TABLE 18F
Domain Analysis of NOV18a

| | | | |
|---|---|---|---|
| Pfam | NOV18a Match Region | Identities/ | Expect |
| Domain | | Similarities for | Value |
| | | the Matched | |
| | | Region | |

### Example 19.

The NOV19 clone was analyzed, and the nucleotide and encoded polypeptide sequences are shown in Table 19A.

TABLE 19A
NOV19 Sequence Analysis

| | | |
|---|---|---|
| NOV19a, CG52988-02 | SEQ ID NO: 475 | 501 bp |
| DNA Sequence | ORF Start: ATG at 25 | ORF Stop: TAG at 358 |

GGGAAGAGACCATGTGTGGGAAATATGAGTGAGCTTGTAAGAACAAGATCCCAATCCTCAGAAAGAGG
AAATGACCAAGAGTCTTCCCAGCCGGTTGGATCTGTGATTGTCCAGGAGCCCACTGAGGAAAAACGTC
AAGAAGAGGAACCACCAACTGATAATCAGGGTATTGCACCTAGTGGGGAGATTGAAAATCAAGCAGTG
CCTGCTTTTCAAGGGCCTGACATGGAAGCTTTTCAACAGGAACTGGCTCTGCTTAAGATAGAGGATGA
GCCTGGAGATGGTCCTGATGTCAGGGAGGGTATTATGCCCACTTTTGATCTCACTAAAGTGCTGGAAG
CAGGTGATGCGCAACCATAGGTTTCAAGCAAGACAAATGAAGACTGAAACCAAGAACGTTATTCTTAA
TCTGGAAATTTGACTGATAATATTCTCTTAATAAAGTTTTAAGTTTTCTGCAAAGAATCCTCAAAAAA
AAAAAAAAAAAAAAAAAAAAAGGG

| | | | |
|---|---|---|---|
| NOV19a, CG52988-02 | SEQ ID NO: 476 | 111 aa | MW at 12106.0kD |
| Protein Sequence | | | |

MSELVRTRSQSSERGNDQESSQPVGSVIVQEPTEEKRQEEEPPTDNQGIAPSGEIENQAVPAFQGPDM
EAFQQELALLKIEDEPGDGPDVREGIMPTFDLTKVLEAGDAQP

| | | |
|---|---|---|
| NOV19b, CG52988-03 | SEQ ID NO: 477 | 374 bp |
| DNA Sequence | ORF Start: ATG at 23 | ORF Stop: TAG at 356 |

GAAGAGACCATGTGTGGGAAATATGAGTGAGCTTGTAAGAGCAAGATCCCAATCCTCAGAAAGAGGAA
ATGACCAAGAGTCTTCCCAGCCGGTTGGATCTGTGATTGTCCAGGAGCCCACTGAGGAAAACGTCA
GAAGAGGAACCACCAACTGATAATCAGGGTATTGCACCTAGTGGGGAGATTGAAAATCAAGCAGTGCC
TGCTTTTCAAGGGCCTGACATGGAAGCTTTTCAACAGGAACTGGCTCTGCTTAAGATAGAGGATGAGC
CTGGAGATGGTCCTGATGTCAGGGAGGGTATTATGCCCACTTTTGATCTCACTAA~GTGCTGGAAGCA
IGGTGATGCGCAACCATAQGTTTCAAGCAAGACAA

| | | | |
|---|---|---|---|
| NOV19b, CG52988-03 | SEQ ID NO: 478 | 111 aa | MW at 12076 0kD |
| Protein Sequence | | | |

MSELVRARSQSSERGNDQESSQPVGSVIVQEPTEEKRQEEEPPTDNQGIAPSGEIENQAVPAFQGPDM
EAFQQELALLKIEDEPGDGPDVREGIMPTEDLTKVLEAGDAQP

| | | |
|---|---|---|
| NOV19c, CG52988-04 | SEQ ID NO: 479 | 1337 bp |
| DNA Sequence | ORF Start: ATG at 1 | ORF Stop: TAG at 334 |

ATGAGTGAGCATGTGAGAACAACATCCCAATCCTCAGAAGAGGAAATGACCAAGAGTCTTCCCAGCC
AGTTGGATCTGTGATTGTCCAGGAGCCCACTGAGGAAAAACGTCAAGAAGAGGAACCACCAACTGATA
ATCAGGGTATTGCACCTAGTGGGGAGATCGAAAATGAAGGAGCACCTGCCGTTCAAGGGCCTGACATG
GAAGCTTTTCAAACAGGAACTGGCTCTGCTTAAGATAGAGGATGAGCCTGGAGATGGTCCTGATGTCAG
GGAGGGGATTATGCCCACTTTTGATCTCACTAAAGTGCTGGAAGCAGGTGATGCGCAACCATAGT

| | | | |
|---|---|---|---|
| NOV19c, CG52988-04 | SEQ ID NO: 480 | 111 a | MW at 11985.8kD |
| Protein Sequence | | | |

MSEAAAATTSQSSERGNDQESSQPVGSVIVQEPTEEKRQEEEPPTDNQGIAPSGEIENEGAPAVQGPDM
EAFQQELALLKIEDEPGDGPDVREGIMPTFDLTKVLEAGDAQP

| | | |
|---|---|---|
| NOV19d, CG52988-06 | SEQ ID NO: 481 | 344 bp |
| DNA Sequence | ORF Start: ATG at 9 | ORF Stop: TAG at 342 |

GTGGAATATGATTGAGCATGTGAGAACAATATCCCAATCCTCAGAAGAGGAAATGACCAAAGAGTCT
AACTGATAATCAGGGTATTGCACCTAGTGGGGAGATCGAAAATGAAGGAGCACCTGCCGTTCAAGGGC
CTGACATGGAAGCTTTTCAACAGGAACTGGCTCTGCTTAAGATAGAGGATGAGCCTGGAGATGGTCCT
GATGTCAGGGAGGGGATTATGCCCACTTTTGATCTCACTAGAGTGCTGGAAGCAGGTGATGCGCAACC
ATAG

| | | | |
|---|---|---|---|
| NOV19d, CG52988-06 | SEQ ID NO: 482 | 111 aa | MW at 12061 .0kD |
| Protein Sequence | | | |

MIEHVRTSQSSERGNDQESSHPVGSVIVQEPTEEKRQEEEPPTDNQGIAPSGEIENEGAPAVQGPDM
EAFQQELALLKIEDEPGDGPDVREGIMPTFDLTRVLEAGDAQP

| | | |
|---|---|---|
| lNOV19e, CG52988-07 | SEQ ID NO: 483 | 1343 bp |
| DNA Sequence | ORF Start: ATG at 8 | ORF Stop: TAG at 341 |

GGGAAATATGAGTGAGCATGTGAGAACAAGATCCCAATCCTCAGAAAGAGGAAATGACCAAGAGTCTT
ACTGATAATCAGGGTATTGCGCCTAGTGGGGAGATCGAAAATGAAGGAGCACCTGCCGTTCAAGGGCC
TGACATGGAAGCTTTTCAACAGGAACTGGCTCTGCTTAAGATAGAGGATGAGCCTGGAGATGGTCCTG
ATGTCAGGGAGGGGATTATGCCCACTTTTGATCTCACTAAAGTGCTGGAAGCAGGTGATGCGCAACCA
TAG

| | | | |
|---|---|---|---|
| NOV19e, CG52988-07 | SEQ ID NO: 484 | 111 aa | MW at 12040.9kD |
| Protein Sequence | | | |

MSEHVRTRSQSSERGNDQESSQPVGSVTVQEPTEEKRQEEEPTDNQGIAGEIENEGAPAVQGPDM
EAFQQELALLKIEDEPGDGPDVREGIMPTFDLTKVLEAGDAQ

| | | |
|---|---|---|
| NOV19f, CG52988-01 | SEQ ID NO: 485 | 475 bp |
| DNA Sequence | ORF Start: ATG at 25 | ORF Stop: TAG at 358 |

AAATGACCAAGAGTCTTCCCAGCCAGTTGGATCTGTGATTGTCCAGGAGCCCACTGAGGAAAAAAACGTC
AAGAAGAGGAACCACCAACTGATAATCAGGGTATTGCACCTAGTGGGGAGATCGAATGAAAGGAGCA
CCTGCCGTTCAAGGGCCTGACATGGAAGCTTTTCACAGGAAACTGGCTCTGCTTAAAGATAGAGGATGA
GCCTGGAGATGGTCCTGATGTCAGGGAGGGGATTATGCCCACTTTTGATCTCACTAAAGTGCTGGAAG
CAGGTGATGCGCAACCATAGGTTTCAAGCAGACATGAAGACTGAACCAGAACGTTATTCTTA14
TCTGGAAATTTGACTGATAATATTCTCTTAATAGTTTTAGTTTTCTGCAGA

| | | | |
|---|---|---|---|
| NOV19f,CG52988-01 | SEQ ID NO: 486 | 111 aa | MW at 12040.9kD |
| Protein Sequence | | | |

MSEHVRTRSQSSERGNDQESSQPVGSVIVQEPTEEKRQEEEPPTDNQGIAPSGEIENEGAPAVQGPDM
IEAFQQELALLKIEDEPGDGPDVREGIMPTFDLTKvLEAGDAQP

| | | |
|---|---|---|
| NOV19g, CG52988-05 | SEQ ID NO: 487 | 1344 bp |
| DNA Sequence | ORF Start: ATG at 9 | ORF Stop: TAG at 342 |

TGGGAAATATGAGTGAGCATGTGAGAACAACATCCCAATCCTCAGAAAGAGGAAATGACCAAGAGTCT
CTGACATGGAAGCTTTTCAACAGGAACTGGCTCTGCTTAAGATAGAGGATGAGCCTGGAGATGGTCCT
GATGTCAGGGAGGGGATTATGCCCACTTTTGATCTCACTAAAGTGCTGGAAGCAGGTGATGCGCAACC
ATAG

| | | | |
|---|---|---|---|
| NOV19g, CG52988-05 | SEQ ID NO: 488 | 111 aaa | MW at 12027.9kD |
| Protein Sequence | | | |

MSEHVRTTSQSSERGNDQESSQPVVSVIVQEPTEEKRQEEEPPTDNQGIApSGEIENEGAPAVQGPDM
EAFQQELALLKIEDEPGDGPDVREGIMPTFDLTKVLEAGDAQP

A ClustalW comparison of the above protein sequences yields the following sequence alignment shown in Table 19B.

TABLE 19B
Comparison of the NOV19 protein sequences.

| | |
|---|---|
| NOV19a | MSELVRTRSQSSERGNDQESSQPVGSVIVQEPTEEKRQEEEPPTDNQGIAPSGEIENQAV |
| NOV19b | MSELVRARSQSSERGNDQESSQPVGSVIVQEPTEEKRQEEEPPTDNQGIAPSGEIENQAV |
| NOV19c | MSEHVRTTSQSSERGNDQESSQPVGSVIVQEPTEEKRQEEEPPTDNQGIAPSGEIENEGA |
| NOV19d | MIEHVRTISQSSERGNDQESSHPVGSVIVQEPTEEKRQEEEPPTDNQGIAPSGEIENEGA |
| NOV19e | MSEHVRTRSQSSERGNDQESSQPVGSVIVQEPTEEKRQEEEPPTDNQGIAPSGEIENEGA |
| NOV19f | MSEHVRTRSQSSERGNDQESSQPVGSVIVQEPTEEKRQEEEPPTDNQGIAPSGEIENEGA |
| NOV19g | MSEHVRTTSQSSERGNDQESSQPVVSVIVQEPTEEKRQEEEPPTDNQGIAPSGEIENEGA |
| NOV19a | PAFQGPDMEAFQQELALLKIEDEPGDGPDVREGIMPTFDLTKVLEAGDAQP |
| NOV19b | PAFQGPDMEAFQQELALLKIEDEPGDGPDVREGIMPTFDLTKVLEAGDAQP |
| NOV19c | PAVQGPDMEAFQQELALLKIEDEPGDGPDVREGIMPTFDLTKVLEAGDAQP |
| NOV19d | PAVQGPDMEAFQQELALLKIEDEPGDGPDVREGIMPTFDLTRVLEAGDAQP |
| NOV19e | PAVQGPDMEAFQQELALLKIEDEPGDGPDVREGIMPTFDLTKVLEAGDAQP |
| NOV19f | PAVQGPDMEAFQQELALLKIEDEPGDGPDVREGIMPTFDLTKVLEAGDAQP |
| NOV19g | PAVQGPDMEAFQQELALLKIEDEPGDGPDVREGIMPTFDLTKVLEAGDAQP |
| NOV19a | (SEQ ID NO: 476) |
| NOV19b | (SEQ ID NO: 478) |
| NOV19c | (SEQ ID NO: 480) |
| NOV19d | (SEQ ID NO: 482) |
| NOV19e | (SEQ ID NO: 484) |
| NOV19f | (SEQ ID NO: 486) |
| NOV19g | (SEQ ID NO: 488) |

Further analysis of the NOV19a protein yielded the following properties shown in Table 19C.

TABLE 19C
Protein Sequence Properties NOV19a

| | |
|---|---|
| SignalP analysis: | No Known Signal Sequence Predicted |
| PSORT II analysis: | |

| PSG: | a new signal peptide prediction method | |
|---|---|---|
| | N-region: | length 8; pos. chg 2; neg. chg 1 |
| | H-region: | length 4; peak value −13.93 |
| | PSG score: | −18.33 |

| | |
|---|---|
| GvH: | von Heijne's method for signal seq. recognition |
| | GvH score (threshold: −2.1): −15.73 |
| | possible cleavage site: between 14 and 15 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 0 | |
| | number of TMS(s) . . . fixed | |
| | PERIPHERAL | Likelihood = 10.87 (at 93) |

| | ALOM score: 10.87 (number of TMSs: 0) | | | |
|---|---|---|---|---|
| MITDISC: | discrimination of mitochondrial targeting seq | | | |
| | R content: | 2 | Hyd Moment(75): | 8.54 |
| | Hyd Moment(95): | 6.16 | G content: | 0 |
| | D/E content: | 2 | S/T content: | 5 |

| | |
|---|---|
| | Score: −3.37 |
| Gavel: | prediction of cleavage sites for mitochondrial preseq |
| | cleavage site motif not found |

NUCDISC: discrimination of nuclear localization signals

| | |
|---|---|
| | pat4: none |
| | pat7: none |
| | bipartite: none |
| | content of basic residues: 7.2% |
| | NLS Score: −0.47 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 89 |
| COIL: | Lupas's algorithm to detect coiled-coil regions |
| | total: 0 residues |

Final Results (k = 9/23):

69.6%: nuclear
26.1%: mitochondrial
4.3%: cytoplasmic

>> prediction for CG52988-02 is nuc (k = 23)

A search of the NOV19a protein against the Geneseq database, a proprietary database that contains sequences published in patents and patent publication, yielded several homologous proteins shown in Table 19D.

TABLE 19D
Geneseq Results for NOV19a

| | | NOV19a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE08593 | Protein encoded by pCR2.1-83350421- | 1 . . . 111 | 110/111 (99%) | 3e−58 |
| | S747-3A insert - Homo sapiens, 111 | 1 . . . 111 | 110/111 (99%) | |
| | aa. [WO200161009-A2, 23-AUG-2001] | | | |
| AAE08582 | Human NOV3 protein - Homo sapiens, | 1 . . . 111 | 110/111 (99%) | 3e−58 |
| | 111 aa. [WO200161009-A2, 23-AUG- | 1 . . . 111 | 110/111 (99%) | |
| | 2001] | | | |
| ABJ19255 | Human cancer/testis antigen - SEQ ID | 1 . . . 111 | 106/111 (95%) | 6e−56 |
| | No 37 - Homo sapiens, 111 aa. | 1 . . . 111 | 107/111 (95%) | |
| | [WO200278526-A2, 10-OCT-2002] | | | |
| AAE08581 | Human NOV2 protein - Homo sapiens, | 1 . . . 111 | 106/111 (95%) | 6e−56 |
| | 111 aa. [WO200161009-A2, 23-AUG- | 1 . . . 111 | 107/111 (95%) | |
| | 2001] | | | |
| ABJ19247 | Human cancer/testis antigen - SEQ ID | 1 . . . 110 | 93/110 (84%) | 7e−47 |
| | No 21 - Homo sapiens, 110 aa. | 1 . . . 109 | 99/110 (89%) | |
| | [WO200278526-A2, 10-OCT-2002] | | | |

In a BLAST search of public sequence databases, the NOV19a protein was found to have homology to the proteins shown in the BLASTP data in Table 19E.

TABLE 19E
Public BLASTP Results for NOV19a

| | | NOV19a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| Q8WWL9 | Page-5 protein - Homo sapiens | 1 . . . 110 | 93/110 | (84%) | 2e−46 |
| | (Human), 130 aa. | 21 . . . 129 | 99/110 | (89%) | |
| Q96GU1 | Hypothetical protein - Homo sapiens | 1 . . . 110 | 93/110 | (84%) | 2e−46 |
| | (Human), 110 aa. | 1 . . . 109 | 99/110 | (89%) | |
| O60829 | G antigen family C 1 protein (Prostate- | 6 . . . 111 | 45/106 | (42%) | 5e−11 |
| | associated gene protein 4) (PAGE-4) | 4 . . . 102 | 53/106 | (49%) | |
| | (PAGE-1) (JM27) (GAGE-9) - Homo | | | | |
| | sapiens (Human), 102 aa. | | | | |
| Q8WTP9 | Placenta-specific gene 6 protein (XAGE- | 8 . . . 111 | 42/108 | (38%) | 3e−10 |
| | 3 protein) (pp9012) - Homo sapiens | 4 . . . 109 | 62/108 | (56%) | |
| | (Human), 111 aa. | | | | |
| Q8WWM1 | XAGE-5 protein - Homo sapiens | 21 . . . 111 | 36/92 | (39%) | 5e−09 |
| | (Human), 108 aa. | 17 . . . 106 | 55/92 | (59%) | |

PFam analysis predicts that the NOV19a protein contains the domains shown in the Table 19F.

TABLE 19F
Domain Analysis of NOV19a

| | | | |
|---|---|---|---|
| Pfam | NOV19a | Identities/ | Expect |
| Domain | Match Region | Similarities | Value |
| | | for the | |
| | | Matched Region | |

### Example 20

The NOV20 clone was analyzed, and the nucleotide and encoded polypeptide sequences are shown in Table 20A.

TABLE 20A
NOV20 Sequence Analysis

| | | |
|---|---|---|
| NOV20a, CG53449-04 | SEQ ID NO: 489 | 2165 bp |

| | | |
|---|---|---|
| DNA Sequence | ORF Start: ATG at 153 | ORF Stop: TGA at 1194 |

AGAACAGCGCAGTTTGCCCTCCGCTCACGCAGAGCCTCTCCGTGGCCTCCGCACCTTGAGCATTAGGC
CAGTTCTCCTCTTCTCTCTAATCCATCCGTCACCTCTCCTGTCATCCGTTTCCATGCCGTGAGGTCCA
TTCACAGAACACATCCATGGCTCTCATGCTCAGTTTGGTTCTGAGTCTCCTCAAGCTGGGATCAGGGC
TTCCTGTCTCCTAAGACCAATGCAGAGGCCATGGAAGTGCGGTTCTTCAGGGGCCAGTTCTCTAGCGT
GGTCCACCTCTACAGGGACGGGAAGGACCAGCCATTTATGCAGATGCCACAGTATCAAGGCAGGACAA
AACTGGTGAAGGATTCTATTGCGGAGGGGCGCATCTCTCTGAGGCTGGAAAACATTACTGTGTTGGAT
GCTGGCCTCTATGGGTGCAGGATTAGTTCCCAGTCTTACTACCAGAAGGCCATCTGGGAGCTACAGGT
GTCAGCACTGGGCTCAGTTCCTCTCATTTCCATCACGGGATATGTTGATAGAGACATCCAGCTACTCT
GTCAGTCCTCGGGCTGGTTCCCCCGGCCCACAGCGAAGTGGAAAGGTCCACAAGGACAGGATTTGTCC
ACAGACTCCAGGACAAACAGAGACATGCATGGCCTGTTTGATGTGGAGATCTCTCTGACCGTCCAAGA
GAACGCCGGGAGCATATCCTGTTCCATGCGGCATGCTCATCTGAGCCGAGAGGTGGAATCCAGGGTAC
AGATAGGAGATACCTTTTTCGAGCCTATATCGTGGCACCTGGCTACCAAAGTACTGGGAATACTCTGC
TGTGGCCTATTTTTTGGCATTGTTGGACTGAAGATTTTCTTCTCCAAATTCCAGTGTAAGCGAGAGAG
AGAAGCATGGGCCGGTGCCTTATTCATGGTTCCAGCAGGGACAGGATCAGAGATGCTCCCACATCCAG
CTGCTTCTCTTCTTCTAGTCCTAGCCTCCAGGGGCCCAGGCCCAAAAAAGGAAAATCCAGGCGGAACT
GGACTGGAGAAGAAAGCACGGACAGGCAGAATTGAGAGACGCCCGGAAACACGCAGTGGAGGTGACTC
TGGATCCAGAGACGGCTCACCCGAAGCTCTGCGTTTCTGATCTGAAAACTGTAACCCATAGAAAAGCT
CCCCAGGAGGTGCCTCACTCTGAGAAGAGATTTACAAGGAAGAGTGTGGTGGCTTCTCAGAGTTTCCA
AGCAGGGAAACATTACTGGGAGGTGGACGGAGGACACAATAAAAGGTGGCGCGTGGGAGTGTGCCGGG
AATGGAGAACATTTGTATTTCACATTAAATCCCCGTTTTATCAGCGTCTTCCCCAGGACCCCACCTAC
AAAAATAGGGGTCTTCCTGGACTATGAGTGTGGGACCATCTCCTTCTTCAACATAAATGACCAGTCCC
TTATTTATACCCTGACATGTCGGTTTGAAGGCTTATTGAGGCCCTACATTGAGTATCCGTCCTATAAT
GAGCAAAATGGAACTCCCATAGTCATCTGCCCAGTCACCCAGGAATCAGAGAAAGAGGCCTCTTGGCA
AAGGGCCTCTGCAATCCCAGAGACAAGCAACAGTGAGTCCTCCTCACAGGCAACCACGCCCTTCCTCC
CCAGGGGTGAAATGTAGGATGAATCACATCCCACATTCTTCTTTAGGGATATTAAGGTCTCTCTCCCA
GATCCAAAGTCCCGCAGCAGCCGGCCAAGGTGGCTTCCAGATGAAGGGGGACTGGCCTGTCCACATGG
CTCCATCTGGCTAAGTGATCTTGAAATACCACCTCTCAGGTGAAGAACCGTCAGGAATTCCCATCTCA
TATCCTAATGGTTTGTTCATTATATTACACTTTCAGTAAAAAAAAAAAAAAAAAAA

NOV20a, CG53449-04

| | | | |
|---|---|---|---|
| Protein Sequence | SEQ ID NO: 490 | 347 aa | MW at 38208.5kD |

MALMLSLVLSLLKLGSGQWQVFGPDKPVQALVGEDAAFSCFLSPKTNAEAMEVRFFRGQFSSVVHLYR
DGKDQPFMQMPQYQGRTKLVKDSIAEGRISLRLENITVLDAGLYGCRISSQSYYQKAIWELQVSALGS
VPLISITGYVDPDIQLLCQSSGWFPRPTAKWKGPQGQDLSTDSRTNRDMHGLFDVEISLTVQENAGSI
SCSMRHAHLSREVESRVQIGDTFFEPISWHLATKVLGILCCGLFFGIVGLKIFFSKFQCKREREAWAG
ALFMVPAGTGSEMLPHPAASLLLVLASRGPGPKKENPGGTGLEKKARTGRIERRPETRSGGDSGSRDG
SPEALRF

| | | |
|---|---|---|
| NOV20b, CG53449-03 | SEQ ID NO: 491 | 1969 bp |

| | | |
|---|---|---|
| DNA Sequence | ORF Start: ATG at 95 | ORF Stop: TAG at 1595 |

AGCATTAGGCCAGTTCTCCTCTTCTCTCTAATCCATCCGTCACCTCTCCTGTCATCCGTTTCCATGCC
GTGAGGTCCATTCACAGAACACATCCATGGCTCTCATGCTCAGTTTGGTTCTGAGTCTCCTCAAGCTG
GGATCAGGGCAGTGGCAGGTGTTTGGGCCAGACAAGCCTGTCCAGGCCTTGGTGGGGGAGGACGCAGC
ATTCTCCTGTTTCCTGTCTCCTAAGACCAATGCAGAGGCCATGGAAGTGCGGTTCTTCAGGGGCCAGT
TCTCTAGCGTGGTCCACCTCTACAGGGACGGGAAGGACCAGCCATTTATGCAGATGCCACAGTATCAA
GGCAGGACAAAACTGGTGAAGGATTCTATTGCGGAGGGGCGCATCTCTCTGAGGCTGGAAAACATTAC
TGTGTTGGATGCTGGCCTCTATGGGTGCAGGATTAGTTCCCAGTCTTACTACCAGAAGGCCATCTGGG
AGCTACAGGTGTCAGCACTGGGCTCAGTTCCTCTCATTTCCATCACGGGATATGTTGATAGAGACATC
CAGCTACTCTGTCAGTCCTCGGGCTGGTTCCCCCGGCCCACAGCGAAGTGGAAAGGTCCACAAGGACA
GGATTTGTCCACAGACTCCAGGACAAACAGAGACATGCATGGCCTGTTTGATGTGGAGATCTCTCTGA
CCGTCCAAGAGAACGCCGGGAGCATATCCTGTTCCATGCGGCATGCTCATCTGAGCCGAGAGGTGGAA
TCCAGGGTACAGATAGGAGATACCTTTTTCGAGCCTATATCGTGGCACCTGGCTACCAAAGTACTGGG
AATACTCTGCTGTGGCCTATTTTTTGGCATTGTTGGACTGAAGATTTTCTTCTCCAAATTCCAGTGGA
AAATCCAGGCGGAACTGGACTGGAGAAGAAAGCACGGACAGGCAGAATTGAGAGACGCCCGGAAACAC
GCAGTGGAGGTGACTCTGGATCCAGAGACGGCTCACCCGAAGCTCTGCGTTTCTGATCTGAAPACTGT
AACCCATAGAAAAGCTCCCCAGGAGGTGCCTCACTCTGAGAAGAGATTTACAAGGAAGAGTGTGGTGG
CTTCTCAGAGTTTCCAAGCAGGGAAACATTACTGGGAGGTGGACGGAGGACACAATAAAAGGTGGCGC
GTGGGAGTGTGCCGGGATGATGTGGACAGGAGGAAGGAGTACGTGACTTTGTCTCCCGATCATGGGTA
CTGGGTCCTCAGACTGAATGGAGAACATTTGTATTTCACATTAAATCCCCGTTTTATCAGCGTCTTCC
CCAGGACCCCACCTACAAAAATAGGGGTCTTCCTGGACTATGAGTGTGGGACCATCTCCTTCTTCAAC
ATAAATGACCAGTCCCTTATTTATACCCTGACATGTCGGTTTGAAGGCTTATTGAGGCCCTACATTGA
GTATCCGTCCTATAATGAGCAAAATGGAACTCCCATAGTCATCTGCCCAGTCACCCAGGAATCAGAGA
ACCACGCCCTTCCTCCCCAGGGGTGAAATGTAGGATGAATCACATCCCACATTCTTCTTTAGGGATAT
TAAGGTCTCTCTCCCAGATCCAAAGTCCCGCAGCAGCCGGCCAAGGTGGCTTCCAGATGAAGGGGGAC
TGGCCTGTCCACATGGGAGTCAGGTGTCATGGCTGCCCTGAGCTGGGAGGGAAGAAGGCTGACATTAC
ATTTAGTTTGCTCTCACTCCATCTGGCTAAGTGATCTTGAAATACCACCTCTCAGGTGAAGAACCGTC
AGGAATTCCCATCTCACAGGCTGTGGTGTAGATTAAGTAGACAAGGAATGTGAATAATGCTTAGATCT
TATTGATGACAGAGTGTATCCTAATGGTTTGTTCATTATATTACACTTTCAGTAAAAAAAAAAAA

NOV20b, CG53449-03

| | | | |
|---|---|---|---|
| Protein Sequence | SEQ ID NO: 492 | 500 aa | MW at 56747.1kD |

MALMLSLVLSLLKLGSGQWQVFGPDKPVQALVGEDAAFSCFLSPKTNAEAMEVRFFRGQFSSVVHLYR
DGKDQPFMQMPQYQGRTKLVKDSIAEGRISLRLENITVLDAGLYGCRISSQSYYQKAIWELQVSALGS
VPLISITGYVDRDIQLLCQSSGWFPRPTAKWKGPQGQDLSTDSRTNRDMHGLFDVEISLTVQENAGSI
SCSMRHAHLSREVESRVQIGDTFFEPISWHLATKVLGILCCGLFFGIVGLKIFFSKFQWKIQAELDWR
HYWEVDGGHNKRWRVGVCRDDVDRRKEYVTLSPDHGYWVLRLNGEHLYFTLNPRFISVFPRTPPTKIG
VFLDYECGTISFFNINDQSLIYTLTCRFEGLLRPYIEYPSYNEQNGTPIVICPVTQESEKEASWQRAS
AIPETSNSESSSQATTPFLPRGEM

| | | |
|---|---|---|
| NOV20c, CG53449-01 | SEQ ID NO: 493 | 252 bp |

| | | |
|---|---|---|
| DNA Sequence | ORF Start: ATG at 1 | ORF Stop: end of sequence |

ATGAACCCGGTACCTCAGATGGAAATGCAGAAATCACCCATGTTCTGCGTCGCTCACTCTGGGAGCTG
TAGACCGGAGCTGTTCCTATTCGGCCATCTTGGCTCCTCCCCCTCACTACAAAGGGTTTTGATGATTT
ATCTTTTGTATGTCTTCCCACAGGGCAGTGGCAGGTGTTTGGGCCAGACAAGCCTGTCCAGGCCTTGG
TGGGGGAGGACGCAGCATTCTCCTGTTTCCTGTCTCCTAAAACCAATG

NOV20c, CG53449-01

| | | | |
|---|---|---|---|
| Protein Sequence | SEQ ID NO: 494 | 84 aa | MW at 9453.1kD |

MNPVPQMEMQKSPMFCVAHSGSCRPELFLFGHLGSSPSLQRVLMYLLYVFPQGSGRCLGQTSLSRPW
WGRTQHSPVSCLLKPM

| | | |
|---|---|---|
| NOV20d, CG53449-02 | SEQ ID NO: 495 | 1495 bp |

| | | |
|---|---|---|
| DNA Sequence | ORF Start: ATG at 1 | ORF Stop: TGA at 1492 |

ATGAACCCGGTACCTCAGATGGAAATGCAGAAATCACCCATGTTCTGCGTCGCTCACTCTGGGAGCTG
TAGACCGGAGCTGTTCCTATTCGGCCATCTTGGCTCCTCCCCCTCACTACAAAGGGTTTTGATGATTT
ATCTTTTGTATGTCTTCCCACAGGGCAGTGGCAGGTGTTTGGGCCAGACAAGCCTGTCCAGGCCTTGG
TGGGGGAGGACGCAGCATTCTCCTGTTTCCTGTCTCCTAAAACCAATGCAGAGGCCATGGAAGTGCGG
TTCTTCAGGGGCCAGTTCTCTAGCGTGGTCCACCTCTACAGGGACGGGAAGGACCAGCCATTTATGCA
GATGCCACAGTATCAAGGCAGGACAAAACTGTGAAGGATTCTATTGCGGAGGGGCGCATCTCTCGAGG
CTGGAAAACATTACTGTGTTGGATGCTGGCCTCTATGGGTGCAGGATTAGTTCCCAGTCTTACTACCA
GAAGGCCATCTGGGAGCTACAGGTGTCAGCACTGGGCTCAGTTCCTCTCATTTCCATCACGGGATATG
TTGATAGAGACATCCAGCTACTCTGTCAGTCCTCGGGCTGGTTCCCCCGGCCCGTGCAAGGAGCCAGC
GTCGTGTTTGTGCCTTGTACACTCCTGTGTCCACCACTGAATATACTGTTTCTGTTTCAGGGAAAATC
CAGGCGGAACTGGGTAAGTATGTGTCATGTCCTGAGCCTCCCACACATGGTTCTCCCGGGTCCCTCCC
TGATCCACAGTTTGAGCCTCTGGACGACCCTGGCTGCAGGCTGGACAGGAAGCACCGACTGGAGAAGA
AAGCACGGACAGGCAGAATTGAGAGACGCCCGGAAACACGCAGTGGAGGTGACTCTGGATCCAGAGAC
GGCTCACCCGAAGCTCTGCGTTTCTGATCTGAAAACTGTAACCCATAGAAAAGCTCCCCAGGAGGTGC
CTCACTCTGAGAAGAGATTTACAAGGAAGAGTGTGGTGGCTTCTCAGAGTTTCCAAGCAGGGAAACAT
TACTGGGAGGTGGACGGAGGACACAATAAAAGGTGGCGCGTGGGAGTGTGCCGGGATGATGTGGACAG
GAGGAAGGAGTACGTGACTTTGTCTCCCGATCATGGGTACTGGGTCCTCAGACTGAATGGAGAACATT
TGTATTTCACATTAAATCCCCGTTTTATCAGCGTCTTCCCCAGGACCCCACCTACAAAAATAGGGGTC
TTCCTGGACTATGAGTGTGGGACCATCTCCTTCTTCAACATAAATGACCAGTCCCTTATTTATACCCT
GACATGTCGGTTTGAAGGCTTATTGAGGCCCTACATTGAGTATCCGTCCTATAATGAGCAAAATGGAA
CTCCCATAGTCATCTGCCCAGTCACCCAGGAATCAGAGAAAGAGGCCTCTTGGCAAAGGGCCTCTGCA
ATCCCAGAGACAAGCAACAGTGAAGTCCTCCTCACAGGCAACCACGCCCTTCCTCCCCAGGGGTGAA

NOV20d, CG53449-02

| | | | |
|---|---|---|---|
| Protein Sequence | SEQ ID NO: 496 | 497 aa | MW at 55724.3kD |

MNPVPQMEMQKSPMFCVAHSGSCRPELFLFGHLGSSPSLQRVLMTYLLYVFPQGSGRCLGQTSLSRPW
WGRTQHSPVSCLLKPMQRPWKCGSSGASSLAWSTSTGTGRTSHLCRCHSIKAGQNCEGFYCGGAHLSR
LENITVLDAGLYGCRISSQSYYQKAIWELQVSALGSVPLISITGYVDRDIQLLCQSSGWFPRPVQGAS
VVFVPCTLLCPPLNILFLFQGKSRRNWVSMCHVLSLPHNVLPGPSLIHSLSLWTTLAAGWTGSTDWRR
KHGQAELRDARKHAVEVTLDPETAHPKLCVSDLKTVTHRKAPQEVPHSEKRFTRKSVVASQSFQAGKH
YWEVDGGHNKRWRVGVCRDDVDRRKEYVTLSPDHGYWVLRLNGEHLYFTLNPRFISVFPRTPPTKIGV
FLDYECGTISFFNINDQSLIYTLTCRFEGLLRPYIEYPSYNEQNGTPIVICPVTQESEKEASWQRASA
IPETSNSEVLLTGNHALPPQG

| | | |
|---|---|---|
| NOV20e, CG53449-05 | SEQ ID NO: 497 | 661 bp |

| | | |
|---|---|---|
| DNA Sequence | ORF Start: at 11 | ORF Stop: at 653 |

CACCAAGCTTCAGTGGCAGGTGTTTGGGCCAGACAAGCCTGTCCAGGCCTTGGTGGGGGAGGACGCAG
CATTCTCCTGTTTCCTGTCTCCTAAGACCAATGCAGAGGCCATGGAAGTGCGGTTCTTCAGGGGCCAG
TTCTCTAGCGTGGTCCACCTCTACAGGGACGGGAAGGACCAGCCATTTATGCAGATGCCACAGTATCA
AGGCAGGACAAAACTGGTGAAGGATTCTATTGCGGAGGGGCGCATCTCTCTGAGGCTGGAAAACATTA
CTGTGTTGGATGCTGGCCTCTATGGGTGCAGGATTAGTTCCCAGTCTTACTACCAGAAGGCCATCTGG
GAGCTACAGGTGTCAGCACTGGGCTCAGTTCCTCTCATTTCCATCACGGGATATGTTGATAGAGACAT
CCAGCTACTCTGTCAGTCCTCGGGCTGGTTCCCCCGGCCCACAGCGAAGTGGAAAGGTCCACAAGGAC
AGGATTTGTCCACAGACTCCAGGACAAACAGAGACATGCATGGCCTGTTTGATGTGGAGATCTCTCTG
ACCGTCCAAGAGAACGCCGGGAGCATATCCTGTTCCATGCGGCATGCTCATCTGAGCCGAGAGGTGGA
ATCCAGGGTACAGATAGGAGATACCTTTTTCGAGCCTATACTCGAGGGC

NOV20e, CG53449-05

| | | | |
|---|---|---|---|
| Protein Sequence | SEQ ID NO: 498 | 214 aa | MW at 24072.0kD |

QWQVFGPDKPVQALVGEDAAFSCFLSPKTNAEAMEVRFFRGQFSSVVHLYRDGKDQPFMQMPQYQGRT
KLVKDSIAEGRISLRLENITVLDAGLYGCRISSQSYYQKAIWELQVSALGSVPLISITGYVDRDIQLL
CQSSGWFPRPTAKWKGPQGQDLSTDSRTNRDMHGLFDVEISLTVQENAGSISCSMRHAHLSREVESRV
QIGDTFFEPI

| | | |
|---|---|---|
| NOV20f, CG53449-06 | SEQ ID NO: 499 | 1066 bp |

| | | |
|---|---|---|
| DNA Sequence | ORF Start: ATG at 17 | ORF Stop: at 1058 |

CACCAAGCTTCCCACCATGGCTCTCATGCTCAGTTTGGTTCTGAGTCTCCTCAAGCTGGGATCAGGGC
AGTGGCAGGTGTTTGGGCCAGACAAGCCTGTCCAGGCCTTGGTGGGGGAGGACGCAGCATTCTCCTGT
TTCCTGTCTCCTAAGACCAATGCAGAGGCCATGGAAGTGCGGTTCTTCAGGGGCCAGTTCTCTAGCGT
GGTCCACCTCTACAGGGACGGGAAGGACCAGCCATTTATGCAGATGCCACAGTATCAAGGCAGGACAA
AACTGGTGAAGGATTCTATTGCGGAGGGGCGCATCTCTCTGAGGCTGGAAAACATTACTGTGTTGGAT
GCTGGCCTCTATGGGTGCAGGATTAGTTCCCAGTCTTACTACCAGAAGGCCATCTGGGAGCTACAGGT
GTCAGCACTGGGCTCAGTTCCTCTCATTTCCATCACGGGATATGTTGATAGAGACATCCAGCTACTCT
GTCAGTCCTCGGGCTGGTTCCCCCGGCCCACAGCGAAGTGGAAAGGTCCACAAGGACAGGATTTGTCC
ACAGACTCCAGGACAAACAGAGACATGCATGGCCTGTTTGATGTGGAGATCTCTCTGACCGTCCAAGA
GAACGCCGGGAGCATATCCTGTTCCATGCGGCATGCTCATCTGAGCCGAGAGGTGGAATCCAGGGTAC
AGATAGGAGATACCTTTTTCGAGCCTATATCGTGGCACCTGGCTACCAAAGTACTGGGAATACTCTGC
TGTGGCCTATTTTTTGGCATTGTTGGACTGAAGATTTTCTTCTCCAAATTCCAGTGTAAGCGAGAGAG
AGAAGCATGGGCCGGTGCCTTATTCATGGTTCCAGCAGGGACAGGATCAGAGATGCTCCCACATCCAG
CTGCTTCTCTTCTTCTAGTCCTAGCCTCCAGGGGCCCAGGCCCAAAAAAGGAAAATCCAGGCGGAACT
GGACTGGAGAAGAAAGCACGGACAGGCAGAATTGAGAGACGCCCGGAAACACGCAGTGGAGGTGACTC
TGGATCCAGAGACGGCTCACCCGAAGCTCTGCGTTTCCTCGAGGGC

NOV20f, CG53449-06

| | | | |
|---|---|---|---|
| Protein Sequence | SEQ ID NO: 500 | 347 aa | MW at 38208.5kD |

MALMLSLVLSLLKLGSGQWQVFGPDKPVQALVGEDAAFSCFLSPKTNAEAMEVRFFRGQFSSVVHLYR
DGKDQPFMQMPQYQGRTKLVKDSIAEGRISLRLENITVLDAGLYGCRISSQSYYQKAIWELQVSALGS
VPLISITGYVDRDIQLLCQSSGWFPRPTAKWKGPQGQDLSTDSRTNRDMHGLFDVEISLTVQENAGSI
SCSMRHAHLSREVESRVQIGDTFFEPISWHLATKVLGILCCGLFFGIVGLKIFFSKFQCKREREAWAG
ALFMVPAGTGSEMLPHPAASLLLVLASRGPGPKKENPGGTGLEKKARTGRIERRPETRSGGDSGSRDG
SPEALRF

| | |
|---|---|
| SEQ ID NO: 501 | 1969 bp |

| | | |
|---|---|---|
| NOV20g, SNP13382434 of | ORF Start: ATG at 95 | ORF Stop: TAG at 1595 |

| | | |
|---|---|---|
| CG53449-03, DNA Sequence | SNP Pos: 396 | SNP Change: T to C |

AGCATTAGGCCAGTTCTCCTCTTCTCTCTAATCCATCCGTCACCTCTCCTGTCATCCGTTTCCATGCC
GTGAGGTCCATTCACAGAACACATCCATGGCTCTCATGCTCAGTTTGGTTCTGAGTCTCCTCAAGCTG
GGATCAGGGCAGTGGCAGGTGTTTGGGCCAGACAAGCCTGTCCAGGCCTTGGTGGGGGAGGACGCAGC
ATTCTCCTGTTTCCTGTCTCCTAAGACCAATGCAGAGGCCATGGAAGTGCGGTTCTTCAGGGGCCAGT
TCTCTAGCGTGGTCCACCTCTACAGGGACGGGAAGGACCAGCCATTTATGCAGATGCCACAGTATCAA
GGCAGGACAAAACTGGTGAAGGATTCTATTGCGGAGGGGCGCATCTCTCTGAGGCCGGAAAACATTAC
TGTGTTGGATGCTGGCCTCTATGGGTGCAGGATTAGTTCCCAGTCTTACTACCAGAAGGCCATCTGGG
AGCTACAGGTGTCAGCACTGGGCTCAGTTCCTCTCATTTCCATCACGGGATATGTTGATAGAGACATC
CAGCTACTCTGTCAGTCCTCGGGCTGGTTCCCCCGGCCCACAGCGAAGTGGAAAGGTCCACAAGGACA
GGATTTGTCCACAGACTCCAGGACAAACAGAGACATGCATGGCCTGTTTGATGTGGAGATCTCTCTGA
CCGTCCAAGAGAACGCCGGGAGCATATCCTGTTCCATGCGGCATGCTCATCTGAGCCGAGAGGTGGAA
TCCAGGGTACAGATAGGAGATACCTTTTTCGAGCCTATATCGTGGCACCTGGCTACCAAAGTACTGGG
AAATCCAGGCGGAACTGGACTGGAGAAGAAAGCACGGACAGGCAGAATTGAGAGACGCCCGGAAACAC
GCAGTGGAGGTGACTCTGGATCCAGAGACGGCTCACCCGAAGCTCTGCGTTTCTGATCTGAAAACTGT
AACCCATAGAAAAGCTCCCCAGGAGGTGCCTCACTCTGAGAAGAGATTTACAAGGAAGAGTGTGGTGG
CTTCTCAGAGTTTCCAAGCAGGGAAACATTACTGGGAGGTGGACGGAGGACACAATAAAAGGTGGCGC
GTGGGAGTGTGCCGGGATGATGTGGACAGGAGGAAGGAGTACGTGACTTTGTCTCCCGATCATGGGTA
CTGGGTCCTCAGACTGAATGGAGAACATTTGTATTTCACATTAAATCCCCGTTTTATCAGCGTCTTCC
CCAGGACCCCACCTACAAAAATAGGGGTCTTCCTGGACTATGAGTGTGGGACCATCTCCTTCTTCAAC
ATAAATGACCAGTCCCTTATTTATACCCTGACATGTCGGTTTGAAGGCTTATTGAGGCCCTACATTGA
GTATCCGTCCTATAATGAGCAAAATGGAACTCCCATAGTCATCTGCCCAGTCACCCAGGAATCAGAGA
AAGAGGCCTCTTGGCAAAGGGCCTCTGCAATCCCAGAGACAAGCAACAGTGAATCCTCCTCACAGGCA
ACCACGCCCTTCCTCCCCAGGGGTGAAATGTAGGATGAATCACATCCCACATTCTTCTTTAGGGATAT
TAAGGTCTCTCTCCCAGATCCAAAGTCCCGCAGCAGCCGGCCAAGGTGGCTTCCAGATGAAGGGGGAC
TGGCCTGTCCACATGGGAGTCAGGTGTCATGGCTGCCCTGAGCTGGGAGGGAAGAAGGCTGACATTAC
ATTTAGTTTGCTCTCACTCCATCTGGCTAAGTGATCTTGAAATACCACCTCTCAGGTGAAGAACCGTC
AGGAATTCCCATCTCACAGGCTGTGGTGTAGATTAAGTAGACAAGGAATGTGAATAATGCTTAGATCT
TATTGATGACAGAGTGTATCCTAATGGTTTGTTCATTATATTACACTTTCAGTAAAAAAAAAAAA

| | | |
|---|---|---|
| NOV20g, SNP13382434 of | SEQ ID NO: 502 | MW at 56731.1kD |

| | | | |
|---|---|---|---|
| CG53449-03, Protein Sequence | SNP Pos: 101 | 500 aa | SNP Change: Leu to Pro |

MALMLSLVLSLLKLGSGQWQVFGPDKPVQALVGEDAAFSCFLSPKTNAEAMEVRFFRGQFSSVVHLYR
DGKDQPFMQMPQYQGRTKLVKDSIAEGRISLRPENITVLDAGLYGCRISSQSYYQKAIWELQVSALGS
VPLISITGYVDRDIQLLCQSSGWFPRPTAKWKGPQGQDLSTDSRTNRDMHGLFDVEISLTVQENAGSI
SCSMRHAHLSREVESRVQIGDTFFEPISWHLATKVLGILCCGLFFGIVGLKIFFSKFQWKIQAELDWR
RKNGQAELRDARKHAVEVTLDPETAHPKLCVSDLKTVTHRKAPQEVPHSEKRFTRKSVVASQSFQAGK
HYWEVDGGHNKRWRVGVCRDDVDRRKEYVTLSPDHGYWVLRLNGEHLYFTLNPRFISVFPRTPPTKIG
VFLDYECGTISFFNINDQSLIYTLTCRFEGLLRPYIEYPSYNEQNGTPIVICPVTQESEKEASWQRAS
AIPETSNSESSSQATTPFLPRGEM

| | |
|---|---|
| SEQ ID NO: 503 | 1969 bp |

| | | |
|---|---|---|
| NOV20h, SNP13382441 of | ORF Start: ATG at 95 | ORF Stop: TAG at 1595 |

| | | |
|---|---|---|
| CG53449-03, DNA Sequence | SNP Pos: 1549 | SNP Change: A to G |

AGCATTAGGCCAGTTCTCCTCTTCTCTCTAATCCATCCGTCACCTCTCCTGTCATCCGTTTCCATGCC
GTGAGGTCCATTCACAGAACACATCCATGGCTCTCATGCTCAGTTTGGTTCTGAGTCTCCTCAAGCTG
GGATCAGGGCAGTGGCAGGTGTTTGGGCCAGACAAGCCTGTCCAGGCCTTGGTGGGGGAGGACGCAGC
ATTCTCCTGTTTCCTGTCTCCTAAGACCAATGCAGAGGCCATGGAAGTGCGGTTCTTCAGGGGCCAGT
TCTCTAGCGTGGTCCACCTCTACAGGGACGGGAAGGACCAGCCATTTATGCAGATGCCACAGTATCAA
GGCAGGACAAAACTGGTGAAGGATTCTATTGCGGAGGGGCGCATCTCTCTGAGGCTGGAAAACATTAC
TGTGTTGGATGCTGGCCTCTATGGGTGCAGGATTAGTTCCCAGTCTTACTACCAGAAGGCCATCTGGG
AGCTACAGGTGTCAGCACTGGGCTCAGTTCCTCTCATTTCCATCACGGGATATGTTGATAGAGACATC
CAGCTACTCTGTCAGTCCTCGGGCTGGTTCCCCCGGCCCACAGCGAAGTGGAAAGGTCCACAAGGACA
GGATTTGTCCACAGACTCCAGGACAAACAGAGACATGCATGGCCTGTTTGATGTGGAGATCTCTCTGA
CCGTCCAAGAGAACGCCGGGAGCATATCCTGTTCCATGCGGCATGCTCATCTGAGCCGAGAGGTGGAA
TCCAGGGTACAGATAGGAGATACCTTTTTCGAGCCTATATCGTGGCACCTGGCTACCAAAGTACTGGG
AATACTCTGCTGTGGCCTATTTTTTGGCATTGTTGGACTGAAGATTTTCTTCTCCAAATTCCAGTGGA
AAATCCAGGCGGAACTGGACTGGAGAAGAAAGCACGGACAGGCAGAATTGAGAGACGCCCGGAAACAC
GCAGTGGAGGTGACTCTGGATCCAGAGACGGCTCACCCGAAGCTCTGCGTTTCTGATCTGAAAACTGT
AACCCATAGAAAAGCTCCCCAGGAGGTGCCTCACTCTGAGAAGAGATTTACAAGGAAGAGTGTGGTGG
CTTCTCAGAGTTTCCAAGCAGGGAAACATTACTGGGAGGTGGACGGAGGACACAATAAAAGGTGGCGC
GTGGGAGTGTGCCGGGATGATGTGGACAGGAGGAAGGAGTACGTGACTTTGTCTCCCGATCATGGGTA
CTGGGTCCTCAGACTGAATGGAGAACATTTGTATTTCACATTAAATCCCCGTTTTATCAGCGTCTTCC
ATAAATGACCAGTCCCTTATTTATACCCTGACATGTCGGTTTGAAGGCTTATTGAGGCCCTACATTGA
GTATCCGTCCTATAATGAGCAAAATGGAACTCCCATAGTCATCTGCCCAGTCACCCAGGAATCAGAGA
AAGAGGCCTCTTGGCAAAGGGCCTCTGCAATCCCAGAGACAAGCAACAGTGAGTCCTCCTCACAGGCA
TGGCCTGTCCACATGGGAGTCAGGTGTCATGGCTGCCCTGAGCTGGGAGGGAAGAAGGCTGACATTAC
ATTTAGTTTGCTCTCACTCCATCTGGCTAAGTGATCTTGAAATACCACCTCTCAGGTGAAGAACCGTC
AGGAATTCCCATCTCACAGGCTGTGGTGTAGATTAAGTAGACAAGGAATGTGAATAATGCTTAGATCT
TATTGATGACAGAGTGTATCCTAATGGTTTGTTCATTATATTACACTTTCAGTAAAAAAAAAAAA

| | | |
|---|---|---|
| NOV20h, SNP13382441 of | SEQ ID NO: 504 | MW at 56747.1kD |

| | | | |
|---|---|---|---|
| CG53449-03, Protein Sequence | 500 aa | SNP Pos: 485 | SNP Change: Glu to Glu |

MALMLSLVLSLLKLGSGQWQVFGPDKPVQALVGEDAAFSCFLSPKTNAEAMEVRFFRGQFSSVVHLYR
DGKDQPFMQMPQYQGRTKLVKDSIAEGRISLRLENITVLDAGLYGCRISSQSYYQKAIWELQVSALGS
VPLISITGYVDRDIQLLCQSSGWFPRPTAKWKGPQGQDLSTDSRTNRDMHGLFDVEISLTVQENAGSI
SCSMRHAHLSREVESRVQIGDTFFEPISWHLATKVLGILCCGLFFGIVGLKIFFSKFQWKIQAELDWR
RKHGQAELRDARKHAVEVTLDPETAHPKLCVSDLKTVTHRKAPQEVPHSEKRFTRKSVVASQSFQAGK
HYWEVDGGHNKRWRVGVCRDDVDRRKEYVTLSPDHGYWVLRLNGEHLYFTLNPRFISVFPRTPPTKIG
VFLDYECGTISFFNINDQSLIYTLTCRFEGLLRPYIEYPSYNEQNGTPIVICPVTQESEKEASWQRAS
AIPETSNSESSSQATTPFLPRGEM

A ClustalW comparison of the above protein sequences yields the following sequence alignment shown in Table 20B.

TABLE 20B
Comparison of the NOV20 protein sequences.

| | |
|---|---|
| NOV20a | MALMLSLVLSLLKLGSGQWQVFGPDKPVQALVGEDAAFSCFLSPKTNAEAMEVRFFRGQF |
| NOV20b | MALMLSLVLSLLKLGSGQWQVFGPDKPVQALVGEDAAFSCFLSPKTNAEAMEVRFFRGQF |
| NOV20c | --MNPVPQMEMQKSPMFCVAHSGSCRPELFLFGHLGSSPSLQRVLMIYLLYVFPQ |
| NOV20d | --MNPVPQMEMQKSPMFCVAHSGSCRPELFLFGHLGSSPSLQRVLMIYLLYVFPQ |
| NOV20e | -----------------QWQVFGPDKPVQALVGEDAAFSCFLSPKTNAEAMEVRFFRGQF |
| NOV20f | MALMLSLVLSLLKLGSGQWQVFGPDKPVQALVGEDAAFSCFLSPKTNAEAMEVRFFRGQF |
| NOV20a | SSVVHLYRDGKDQPFMQMPQYQGRTKLVKDSIAEGRIS---------------------- |
| NOV20b | SSVVHLYRDGKDQPFMQMPQYQGRTKLVKDSIAEGRIS---------------------- |
| NOV20c | GSGRCLGQTSLSRPWWGRTQHSPVSCLLKPM----------------------------- |
| NOV20d | GSGRCLGQTSLSRPWWGRTQHSPVSCLLKPMQRPWKCGSSGASSLAWSTSTGTGRTSHLC |
| NOV20e | SSVVHLYRDGKDQPFMQMPQYQGRTKLVKDSIAEGRIS---------------------- |
| NOV20f | SSVVHLYRDGKDQPFMQMPQYQGRTKLVKDSIAEGRIS---------------------- |
| NOV20a | ---------------------LRLENITVLDAGLYGCRISSQSYYQKAIWELQVSALGSV |
| NOV20b | ---------------------LRLENITVLDAGLYGCRISSQSYYQKAIWELQVSALGSV |
| NOV20c | ------------------------------------------------------------ |
| NOV20d | RCHSIKAGQNCEGFYCGGAHLSRLENITVLDAGLYGCRISSQSYYQKAIWELQVSALGSV |
| NOV20e | ---------------------LRLENITVLDAGLYGCRISSQSYYQKAIWELQVSALGSV |
| NOV20f | ---------------------LRLENITVLDAGLYGCRISSQSYYQKAIWELQVSALGSV |
| NOV20a | PLISITGYVDRDIQLLCQSSGWFPRPTAKWKGPQGQDLSTDSRTNRDMHGLFDVEISLTV |
| NOV20b | PLISITGYVDRDIQLLCQSSGWFPRPTAKWKGPQGQDLSTDSRTNRDMHGLFDVEISLTV |
| NOV20c | ------------------------------------------------------------ |
| NOV20d | PLISITGYVDRDIQLLCQSSGWFPRPVQGAS------VVFVPCTLLCPPLNILFLFQGKS |
| NOV20e | PLISITGYVDRDIQLLCQSSGWFPRPTAKWKGPQGQDLSTDSRTNRDMHGLFDVEISLTV |
| NOV20f | PLISITGYVDRDIQLLCQSSGWFPRPTAKWKGPQGQDLSTDSRTNRDMHGLFDVEISLTV |
| NOV20a | QENAGSISCSMRHAHLSREVESRVQIGDTFFEPISWHLATKVLGILCCGLFFGIVGLKIF |
| NOV20b | QENAGSISCSMRHAHLSREVESRVQIGDTFFEPISWHLATKVLGILCCGLFFGIVGLKIF |
| NOV20c | ------------------------------------------------------------ |
| NOV20d | RRNWVSMCHVLSLPHMVLPGPSLIHSLSLWTTLAAGWTGSTDWRRKHGQAELRDARKHAV |
| NOV20e | QENAGSISCSMRHAHLSREVESRVQIGDTFFEPI-------------------------- |
| NOV20f | QENAGSISCSMRHAHLSREVESRVQIGDTFFEPISWHLATKVLGILCCGLFFGIVGLKIF |
| NOV20a | FSKFQCKRERE--------------------------------AWAGALFMVPAGTGSEM |
| NOV20b | FSKFQWKIQAELDWRRKHGQAELRDARKHAVEVTLDPETAHPKLCVSDLKTVTHRKAPQE |
| NOV20c | ------------------------------------------------------------ |
| NOV20d | EVTLDPETAHP-------------------------------KLCVSDLKTVTHRKAPQE |
| NOV20e | ------------------------------------------------------------ |
| NOV20f | FSKFQCKRERE--------------------------------AWAGALFMVPAGTGSEM |
| NOV20a | LPHP---AASLLLVLASRGPGPKKENPGGTGLEKKARTGRIERRPETRSGGDSGSRDGSP |
| NOV20b | VPHSEKRFTRKSVVASQSFQAGKHYWEVDGGHNKRWRVGVCRDDVDRRKEYVTLSPDHGY |
| NOV20c | ------------------------------------------------------------ |
| NOV20d | VPHSEKRFTRKSVVASQSFQAGKHYWEVDGGHNKRWRVGVCRDDVDRRKEYVTLSPDHGY |
| NOV20e | ------------------------------------------------------------ |
| NOV20f | LPHP---AASLLLVLASRGPGPKKENPGGTGLEKKARTGRIERRPETRSGGDSGSRDGSP |
| NOV20a | EALRF------------------------------------------------------- |
| NOV20b | WVLRLNGEHLYFTLNPRFISVFPRTPPTKIGVFLDYECGTISFFNINDQSLIYTLTCRFE |
| NOV20c | ------------------------------------------------------------ |
| NOV20d | WVLRLNGEHLYFTLNPRFISVFPRTPPTKIGVFLDYECGTISFFNINDQSLIYTLTCRFE |
| NOV20e | ------------------------------------------------------------ |
| NOV20f | EALRF------------------------------------------------------- |
| NOV20a | ------------------------------------------------------------ |
| NOV20b | GLLRPYIEYPSYNEQNGTPIVICPVTQESEKEASWQRASAIPETSNSESSSQATTPFLPR |
| NOV20c | ------------------------------------------------------------ |
| NOV20d | GLLRPYIEYPSYNEQNGTPIVICPVTQESEKEASWQRASAIPETSNSEVLLTGNHALPPQ |
| NOV20e | ------------------------------------------------------------ |
| NOV20f | ------------------------------------------------------------ |
| NOV20a | --- |
| NOV20b | GEM |
| NOV20c | --- |
| NOV20d | G-- |
| NOV20e | --- |
| NOV20f | --- |
| NOV20a | (SEQ ID NO: 490) |
| NOV20b | (SEQ ID NO: 492) |
| NOV20c | (SEQ ID NO: 494) |
| NOV20d | (SEQ ID NO: 496) |
| NOV20e | (SEQ ID NO: 498) |
| NOV20f | (SEQ ID NO: 500) |

Further analysis of the NOV20a protein yielded the following properties shown in Table 20C.

TABLE 20C
Protein Sequence Properties NOV20a

| | |
|---|---|
| SignalP analysis: | Cleavage site between residues 18 and 19 |
| PSORT II analysis: | |

| PSG: | a new signal peptide prediction method | |
|---|---|---|
| | N-region: | length 0; pos. chg 0; neg. chg 0 |
| | H-region: | length 12; peak value 10.45 |
| | PSG score: | 6.05 |

| | |
|---|---|
| GvH: | von Heijne's method for signal seq. recognition |
| | GvH score (threshold: −2.1): −0.59 |
| | possible cleavage site: between 17 and 18 |

>>> Seems to have a cleavable signal peptide (1 to 17)

| ALOM: | Klein et al's method for TM region allocation | | |
|---|---|---|---|
| | Init position for calculation: 18 | | |
| | Tentative number of TMS(s) for the threshold 0.5: 1 | | |
| | Number of TMS(s) for threshold 0.5: 1 | | |
| | INTEGRAL | Likelihood = | Transmembrane 242-258 |
| | | −7.06 | |

| | |
|---|---|
| PERIPHERAL | Likelihood = 2.17 (at 126) |

| | |
|---|---|
| | ALOM score: −7.06 (number of TMSs: 1) |
| MTOP: | Prediction of membrane topology (Hartmann et al.) |
| | Center position for calculation: 8 |
| | Charge difference: 0.0 C(1.0)-N(1.0) |
| | N >= C: N-terminal side will be inside |

>>> membrane topology: type 1a (cytoplasmic tail 259 to 347)

| MITDISC: | discrimination of mitochondrial targeting seq | | | |
|---|---|---|---|---|
| | R content: | 0 | Hyd Moment(75): | 1.22 |
| | Hyd Moment(95): | 2.65 | G content: | 3 |
| | D/E content: | 1 | S/T content: | 3 |

| | |
|---|---|
| | Score: −6.60 |
| Gavel: | prediction of cleavage sites for mitochondrial preseq |
| | cleavage site motif not found |

NUCDISC: discrimination of nuclear localization signals

| | |
|---|---|
| | pat4: none |
| | pat7: none |
| | bipartite: none |
| | content of basic residues: 11.8% |
| | NLS Score: −0.47 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: found

LL at 293

checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 76.7 |
| COIL: | Lupas' s algorithm to detect coiled-coil regions |
| | total: 0 residues |

Final Results (k = 9/23):

44.4%: endoplasmic reticulum
22.2%: Golgi
22.2%: extracellular, including cell wall
11.1%: plasma membrane

>> prediction for CG53449-04 is end (k = 9)

A search of the NOV20a protein against the Geneseq database, a proprietary database that contains sequences published in patents and patent publication, yielded several homologous proteins shown in Table 20D.

TABLE 20D
Geneseq Results for NOV20a

| | | NOV20a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU83681 | Human PRO protein, Seq ID No 180 - | 1 . . . 268 | 264/268 (98%) | e−152 |
| | Homo sapiens, 500 aa. | 1 . . . 268 | 265/268 (98%) | |
| | [WO200208288-A2, 31-JAN-2002] | | | |
| ABB84917 | Human PRO1347 protein sequence | 1 . . . 268 | 264/268 (98%) | e−152 |
| | SEQ ID NO: 202 - Homo sapiens, 500 | 1 . . . 268 | 265/268 (98%) | |
| | aa. [WO200200690-A2, 03-JAN-2002] | | | |
| AAE20317 | Human B7-H13 protein #1 - Homo | 1 . . . 268 | 264/268 (98%) | e−152 |
| | sapiens, 461 aa. [WO200202587-A1 , | 1 . . . 268 | 265/268 (98%) | |
| | 10-JAN-2002] | | | |
| ABB95523 | Human angiogenesis related protein | 1 . . . 268 | 264/268 (98%) | e−152 |
| | PRO1347 SEQ ID NO: 202 - Homo | 1 . . . 268 | 265/268 (98%) | |
| | sapiens, 500 aa. [WO200208284-A2, | | | |
| | 31-JAN-2002] | | | |
| ABG95892 | Human secreted/transmembrane | 1 . . . 268 | 264/268 (98%) | e−152 |
| | protein PRO1347 - Homo sapiens, 500 | 1 . . . 268 | 265/268 (98%) | |
| | aa. [US2002119130-A1, 29-AUG-2002] | | | |

In a BLAST search of public sequence databases, the NOV20a protein was found to have homology to the proteins shown in the BLASTP data in Table 20E.

TABLE 20E
Public BLASTP Results for NOV20a

| | | NOV20a | | | |
|---|---|---|---|---|---|
| Protein | | Residues/ | Identities/ | | |
| Accession | | Match | Similarities for the | | Expect |
| Number | Protein/Organism/Length | Residues | Matched Portion | | Value |
| Q9H730 | Hypothetical protein FLJ21458 - | 1 . . . 347 | 347/347 | (100%) | 0.0 |
| | Homo sapiens (Human), 347 aa. | 1 . . . 347 | 347/347 | (100%) | |
| Q9Y2C7 | Butyrophilin like receptor - Homo | 1 . . . 157 | 111/157 | (70%) | 5e−55 |
| | sapiens (Human), 432 aa. | 1 . . . 157 | 126/157 | (79%) | |
| O73716 | B-G-like protein - Grus americana | 7 . . . 268 | 103/266 | (38%) | 3e−45 |
| | (whooping crane), 280 aa. | 17 . . . 280 | 154/266 | (57%) | |
| Q96DM5 | Hypothetical protein FLJ32535 - | 2 . . . 252 | 104/259 | (40%) | 3e−44 |
| | Homo sapiens (Human), 315 aa. | 19 . . . 277 | 159/259 | (61%) | |
| P18892 | Butyrophilin precursor (BT) - Bos | 3 . . . 249 | 96/254 | (37%) | 3e−42 |
| | taurus (Bovine), 526 aa. | 13 . . . 266 | 153/254 | (59%) | |

PFam analysis predicts that the NOV20a protein contains the domains shown in the Table 20F.

TABLE 20F
Domain Analysis of NOV20a

| | | | |
|---|---|---|---|
| Pfam | NOV20a | Identities/ | Expect |
| Domain | Match Region | Similarities | Value |
| | | for the | |
| | | Matched Region | |

### Example 21

The NOV21 clone was analyzedi and the nucleotide and encoded polypeptide sequences are shown in Table 21A.

TABLE 21A
NOV21 Sequence Analysis

| | | |
|---|---|---|
| NOV21a, CG53908-01 | SEQ ID NO: 505 | 2881 bp |

| | | |
|---|---|---|
| DNA Sequence | ORF Start: ATG at 87 | ORF Stop: TGA at 2784 |

AGCTGGGGCTCCGGGCTGAGGCGCTAAAGCCGCCCTCCCGCCCGCGGGGCCCCGCGCCCGGCCCGCCC
GCCTGCCCGCCCGCGGCCATGGCCGTCCGGCCCGGCCTGTGGCCAGCGCTCCTGGGCATAGTCCTCGC
CGGACCTGCTTCCCCACTTCCTGGTGGAGCCCGAGGATGTGTACATCGTCAAGAACAAGCCAGTGCTG
CTTGTGTGCAAGGCCGTGCCCGCCACGCAGATCTTCTTCAAGTGCAACGGGGAGTGGGTGCGCCAGGT
GGACCACGTGATCGAGCGCAGCACAGACGGGAGCAGTGGTGAGCCGACCATGGAGGTCCGCATTAATG
TCTCAAGGCAGCAGGTCGAGAAGGTGTTCGGGCTGGAGGAATACTGGTGCCAGTGCGTGGCATGGAGC
TCCTCGGGCACCACCAAGAGTCAGAAGGCCTACATCCGCATAGCCAGATTGCGCAAGAACTTCGAGCA
GGAGCCGCTGGCCAAGGAGGTGTCCCTGGAGCAGGGCATCGTGCTGCCCTGCCGTCCACCGGAGGGAA
TCCCTCCAGCCGAGGTGGAGTGGCTCCGGAACGAGGACCTGGTGGACCCGTCCCTGGACCCCAATGTA
TACATCACGCGGGAGCACAGCCTGGTGGTGCGACAGGCCCGCCTTGCTGACACGGCCAACTACACCTG
CGTGGCCAAGAACATCGTGGCACGTCGCCGCAGCGCCTCCGCTGCTGTCATCGTCTACGTGAACGGTG
GGTGGTCGACGTGGACCGAGTGGTCCGTCTGCAGCGCCAGCTGTGGGCGCGGCTGGCAGAAACGGAGC
CGGAGCTGCACCAACCCGGCGCCTCTCAACGGGGGCGCTTTCTGTGAGGGGCAGAATGTCCATGACCG
CACCGTCTCCTCTCTGCTTGTCTCTGTGGACGGCAGCTGGAGCCCGTGGAGCAAGTGGTCGGCCTGTG
GGCTGGACTGCACCCACTGGCGGAGCCGTGAGTGCTCTGACCCAGCACCCCGCAACGGAGGGGAGGAG
TGCCAGGGCACTGACCTGGACACCCGCAACTGTACCAGTGACCTCTGTGTACACAGTGCTTCTGGCCC
TGAGGACGTGGCCCTCTATGTGGGCCTCATCGCCGTGGCCGTCTGCCTGGTCCTGCTGCTGCTTGTCC
TCATCCTCGTTTATTGCCGGAAGAAGGAGGGGCTGGACTCAGATGTGGCTGACTCGTCCATTCTCACC
TCAGGCTTCCAGCCCGTCAGCATCAAGCCCAGCAAAGCAGACAACCCCCATCTGCTCACCATCCAGCC
GGACCTCAGCACCACCACCACCTACCAGGGCAGTCTCTGTCCCCGGCAGGATGGGCCCAGCCCCAAGT
TCCAGCTCACCAATGGGCACCTGCTCAGCCCCCTGGGTGGCGGCCGCCACACACTGCACCACAGCTCT
CCCACCTCTGAGGCCGAGGAGTTCGTCTCCCGCCTCTCCACCCAGAACTACTTCCGCTCCCTGCCCCG
AGGCACCAGCAACATGACCTATGGGACCTTCAACTTCCTCGGGGGCCGGCTGATGATCCCTAATACAG
GTATCAGCCTCCTCATCCCCCCAGATGCCATACCCCGAGGGAAGATCTATGAGATCTACCTCACGCTG
CACAAGCCGGAAGACGTGAGGTTGCCCCTAGCTGGCTGTCAGACCCTGCTGAGTCCCATCGTTAGCTG
TGGACCCCCTGGCGTCCTGCTCACCCGGCCAGTCATCCTGGCTATGGACCACTGTGGGGAGCCCAGCC
CTGACAGCTGGAGCCTGCGCCTCAAAAAGCAGTCGTGCGAGGGCAGCTGGGAGCAGGATGTGCTGCAC
CTGGGCGAGGAGGCGCCCTCCCACCTCTACTACTGCCAGCTGGAGGCCAGTGCCTGCTACGTCTTCAC
CGAGCAGCTGGGCCGCTTTGCCCTGGTGGGAGAGGCCCTCAGCGTGGCTGCCGCCAAGCGCCTCAAGC
TGCTTCTGTTTGCGCCGGTGGCCTGCACCTCCCTCGAGTACAACATCCGGGTCTACTGCCTGCATGAC
ACCCACGATGCACTCAAGGAGGTGGTGCAGCTGGAGAAGCAGCTGGGGGGACAGCTGATCCAGGAGCC
ACGGGTCCTGCACTTCAAGGACAGTTACCACAACCTGCGCCTATCCATCCACGATGTGCCCAGCTCCC
TGTGGAAGAGTAAGCTCCTTGTCAGCTACCAGGAGATCCCCTTTTATCACATCTGGAATGGCACGCAG
CGGTACTTGCACTGCACCTTCACCCTGGAGCGTGTCAGCCCCAGCACTAGTGACCTGGCCTGCAAGCT
GTGGGTGTGGCAGGTGGAGGGCGACGGGCAGAGCTTCAGCATCAACTTCAACATCACCAAGGACACAA
GGTTTGCTGAGCTGCTGGCTCTGGAGAGTGAAGCGGGGGTCCCAGCCCTGGTGGGCCCCAGTGCCTTC
AAGATCCCCTTCCTCATTCGGCAGAAGATAATTTCCAGCCTGGACCCACCCTGTAGGCGGGGTGCCGA
CTGGCGGACTCTGGCCCAGAAACTCCACCTGGACAGCCATCTCAGCTTCTTTGCCTCCAAGCCCAGCC
CCACAGCCATGATCCTCAACCTGTGGGAGGCGCGGCACTTCCCCAACGGCAACCTCAGCCAGCTGGCT
GCAGCAGTGGCTGGACTGGGCCAGCCAGACGCTGGCCTCTTCACAGTGTCGGAGGCTGAGTGCTGAGG
CCGGCCAGGCCCGACACCTACACTCTCACCAGCTTTGGCACCCACCAAGGACAGGCAGAAGCCGGACA
GGGGCCCTTCCCCACACCGGGGAGA

NOV21a, CG53908-01

| | | | |
|---|---|---|---|
| Protein Sequence | SEQ ID NO: 506 | 899 aa | MW at 99063.0kD |

MAVRPGLWPALLGIVLAAWLRGSGAQQSATVANPVPGANPDLLPHFLVEPEDVYIVKNKPVLLVCKAV
PATQIFFKCNGEWVRQVDHVIERSTDGSSGEPTMEVRINVSRQQVEKVFGLEEYWCQCVAWSSSGTTK
SQKAYIRIARLRKNFEQEPLAKEVSLEQGIVLPCRPPEGIPPAEVEWLRNEDLVDPSLDPNVYITREH
SLVVRQARLADTANYTCVAKNIVARRRSASAAVIVYVNGGWSTWTEWSVCSASCGRGWQKRSRSCTNP
APLNGGAFCEGQNVHDRTVSSLLVSVDGSWSPWSKWSACGLDCTHWRSRECSDPAPRNGGEECQGTDL
DTRNCTSDLCVHSASGPEDVALYVGLIAVAVCLVLLLLVLILVYCRKKEGLDSDVADSSILTSGFQPV
SIKPSKADNPHLLTIQPDLSTTTTYQGSLCPRQDGPSPKFQLTNGHLLSPLGGGRHTLHHSSPTSEAE
EFVSRLSTQNYFRSLPRGTSNMTYGTFNFLGGRLMIPNTGISLLIPPDAIPRGKIYEIYLTLHKPEDV
RLPLAGCQTLLSPIVSCGPPGVLLTRPVILAMDHCGEPSPDSWSLRLKKQSCEGSWEQDVLHLGEEAP
SHLYYCQLEASACYVFTEQLGRFALVGEALSVAAAKRLKLLLFAPVACTSLEYNIRVYCLHDTHDALK
EVVQLEKQLGGQLIQEPRVLHFKDSYHNLRLSIHDVPSSLWKSKLLVSYQEIPFYHIWNGTQRYLHCT
FTLERVSPSTSDLACKLWVWQVEGDGQSFSINFNITKDTRFAELLALESEAGVPALVGPSAFKIPFLI
RQKIISSLDPPCRRGADWRTLAQKLHLDSHLSFFASKPSPTAMILNLWEARHFPNGNLSQLAAAVAGL
GQPDAGLFTVSEAEC

| | | |
|---|---|---|
| NOV21b, CG53908-02 | SEQ ID NO: 507 | 993 bp |

| | | |
|---|---|---|
| DNA Sequence | ORF Start: at 1 | ORF Stop: end of sequence |

CAGCAGAGTGCCACCGTGGCCAACCCAGTGCCTGGTGCCAACCCGGACCTGCTTCCCCACTTCCTGGT
GGAGCCCGAGGATGTGTACATCGTCAAGAACAAGCCAGTGCTGCTTGTGTGCAAGGCCGTGCCCGCCA
CGCAGATCTTCTTCAAGTGCAACGGGGAGTGGGTGCGCCAGGTGGACCACGTGATCGAGCGCAGCACA
GACGGGAGCAGTGGTGAGCCGACCATGGAGGTCCGCATTAATGTCTCAAGGCAGCAGGTCGAGAAGGT
GTTCGGGCTGGAGGAATACTGGTGCCAGTGCGTGGCATGGAGCTCCTCGGGCACCACCAAGAGTCAGA
AGGCCTACATCCGCATAGCCAGATTGCGCAAGAACTTCGAGCAGGAGCCGCTGGCCAAGGAGGTGTCC
CTGGAGCAGGGCATCGTGCTGCCCTGCCGTCCACCGGAGGGCATCCCTCCAGCCGAGGTGGAGTGGCT
CCGGAACGAGGACCTGGTGGACCCGTCCCTGGACCCCAATGTATACATCACGCGGGAGCACAGCCTGG
TGGTGCGACAGGCCCGCCTTGCTGACACGGCCAACTACACCTGCGTGGCCAAGAACATCGTGGCACGT
CGCCGCAGCGCCTCCGCTGCTGTCATCGTCTACGTGAACGGTGGGTGGTCGACGTGGACCGAGTGGTC
CGTCTGCAGCGCCAGCTGTGGGCGCGGCTGGCAGAAACGGAGCCGGAGCTGCACCAACCCGGCGCCTC
TCAACGGGGGCGCTTTCTGTGAGGGGCAGAATGTCCATGACCGCACCGTCTCCTCTCTGCTTGTCTCT
GTGGACGGCAGCTGGAGCCCGTGGAGCAAGTGGTCGGCCTGTGGGCTGGACTGCACCCACTGGCGGAG
CCGTGAGTGCTCTGACCCAGCACCCCGCAACGGAGGGGAGGAGTGCCAGGGCACTGACCTGGACACCC
GCAACTGTACCAGTGACCTCTGTGTACACAGTGCTTCTGGC

NOV21b, CG53908-02

| | | | |
|---|---|---|---|
| Protein Sequence | SEQ ID NO: 508 | 331 aa | MW at 36604.8kD |

QQSATVANPVPGANPDLLPHFLVEPEDVYIVKNKPVLLVCKAVPATQIFFKCNGEWVRQVDHVIERST
DGSSGEPTMEVRINVSRQQVEKVFGLEEYWCQCVAWSSSGTTKSQKAYIRIARLRKNFEQEPLAKEVS
LEQGIVLPCRPPEGIPPAEVEWLRNEDLVDPSLDPNVYITREHSLVVRQARLADTANYTCVAKNIVAR
RRSASAAVIVYVNGGWSTWTEWSVCSASCGRGWQKRSRSCTNPAPLNGGAFCEGQNVHDRTVSSLLVS
VDGSWSPWSKWSACGLDCTHWRSRECSDPAPRNGGEECQGTDLDTRNCTSDLCVHSASG

| | | |
|---|---|---|
| NOV21c, 306075989 | SEQ ID NO: 509 | 1009 bp |

| | | |
|---|---|---|
| DNA Sequence | ORF Start: at 2 | ORF Stop: end of sequence |

CACCGGATCCCAGCAGAGTGCCACCGTGGCCAACCCAGTGCCTGGTGCCAACCCGGACCTGCTTCCCC
ACTTCCTGGTGGAGCCCGAGGATGTGTACATCGTCAAGAACAAGCCAGTGCTGCTTGTGTGCAAGGCC
GTGCCCGCCACGCAGATCTTCTTCAAGTGCAACGGGGAGTGGGTGCGCCAGGTGGACCACGTGATCGA
GCGCAGCACAGACGGGAGCAGTGGGCTGCCCACCATGGAGGTCCGCATTAATGTCTCAAGGCAGCAGG
TCGAGAAGGTGTTCGGGCTGGAGGAATACTGGTGCCAGTGCGTGGCATGGAGCTCCTCGGGCACCACC
AAGAGTCAGAAGGCCTACATCCGCATAGCCTATTTGCGCAAGAACTTCGAGCAGGAGCCACTGGCCAA
GGAGGTGTCCCTGGAGCAGGGCATCGTGCTGCCCTGCCGTCCACCGGAGGGCATCCCTCCAGCCGAGG
TGGAGTGGCTCCGGAACGAGGACCTGGTGGACCCGTCCCTGGACCCCAATGTATACATCACGCGGGAG
CACAGCCTGGTGGTGCGACAGGCCCGCCTTGCTGACACGGCCAACTACACCTGCGTGGCCAAGAACAT
CGTGGCACGTCGCCGCAGCGCCTCCGCTGCTGTCATCGTCTACGTGAACGGTGGGTGGTCGACGTGGA
CCGAGTGGTCCGTCTGCAGCGCCAGCTGTGGGCGCGGCTGGCAGAAACGGAGCCGGAGCTGCACCAAC
CCGGCGCCTCTCAACGGGGGCGCTTTCTGTGAGGGGCAGAATGTCCAGAAAACAGCCTGCGCCACCCT
GTGCCCAGTGGACGGCAGCTGGAGCCCGTGGAGCAAGTGGTCGGCCTGTGGGCTGGACTGCACCCACT
GGCGGAGCCGTGAGTGCTCTGACCCAGCACCCCGCAACGGAGGGGAGGAGTGCCAGGGCACTGACCTG
GACACCCGCAACTGTACCAGTGACCTCTGTGTACACAGTGCTTCTGGCGAATTCGGC

NOV21c, 306075989

| | | | |
|---|---|---|---|
| Protein Sequence | SEQ ID NO: 510 | 336 aa | MW at 36996.3kD |

TGSQQSATVANPVPGANPDLLPHFLVEPEDVYIVKNKPVLLVCKAVPATQIFFKCNGEWVRQVDHVIE
RSTDGSSGLPTMEVRINVSRQQVEKVFGLEEYWCQCVAWSSSGTTKSQKAYIRIAYLRKNFEQEPLAK
EVSLEQGIVLPCRPPEGIPPAEVEWLPNEDLVDPSLDPNVYITREHSLVVRQARLADTANYTCVAKNI
VARRRSASAAVIVYVNGGWSTWTEWSVCSASCGRGWQKRSRSCTNPAPLNGGAFCEGQNVQKTACATL
CPVDGSWSPWSKWSACGLDCTHWRSRECSDPAPRNGGEECQGTDLDTRNCTSDLCVHSASGEFG

| | | |
|---|---|---|
| NOV21d, CG53908-03 | SEQ ID NO: 511 | 1009 bp |

| | | |
|---|---|---|
| DNA Sequence | ORF Start: at 1 | ORF Stop: at 1006 |

CAGCAGAGTGCCACCGTGGCCAACCCAGTGCCTGGTGCCAACCCGGACCTGCTTCCCCACTTCCTGGT
GGAGCCCGAGGATGTGTACATCGTCAAGAACAAGCCAGTGCTGCTTGTGTGCAAGGCCGTGCCCGCCA
GACGGGAGCAGTGGTGAGCCGACCATGGAGGTCCGCATTAATGTCTCAAGGCAGCAGGTCGAGAAGGT
GTTCGGGCTGGAGGAATACTGGTGCCAGTGCGTGGCATGGAGCTCCTCGGGCACCACCAAGAGTCAGA
AGGCCTACATCCGCATAGCCAGATTGCGCAAGAACTTCGAGCAGGAGCCGCTGGCCAAGGAGGTGTCC
CTGGAGCAGGGCATCGTGCTGCCCTGCCGTCCACCGGAGGGCATCCCTCCAGCCGAGGTGGAGTGGCT
CCGGAACGAGGACCTGGTGGACCCGTCCCTGGACCCCAATGTATACATCACGCGGGAGCACAGCCTGG
TGGTGCGACAGGCCCGCCTTGCTGACACGGCCAACTACACCTGCGTGGCCAAGAACATCGTGGCACGT
CGCCGCAGCGCCTCCGCTGCTGTCATCGTCTACGTGAACGGACGGCAGCTGGAGCCCGTGGAGCAAGT
GGTCGGCCTGTGGGCTGGACTGCACCCACTGGCGGAGCCGTGAGTGCTCTGACCCAGCACCCCGCAAC
GGAGGGGAGGAGTGCCAGGGCACTGACCTGGACACCCGCAACTGTACCAGTGACCTCTGTGTACACAG
TGCTGCTTGTCCTCATCCTCGTTTATTGCCGGAAGAAGGAGGGGCTGGACTCAGATGTGGCTGACTCG
TCCATTCTCACCTCAGGCTTCCAGCCCGTCAGCATCAAGCCCAGCAAAGCAGACAAC

NOV21d, CG53908-03

| | | | |
|---|---|---|---|
| Protein Sequence | SEQ ID NO: 512 | 335 aa | MW at 36949.6kD |

QQSATVANPVPGANPDLLPHFLVEPEDVYIVKNKPVLLVCKAVPATQIFFKCNGEWVRQVDHVIERST
DGSSGEPTMEVRINVSRQQVEKVFGLEEYWCQCVAWSSSGTTKSQKAYIRIARLRKNFEQEPLAKEVS
LEQGIVLPCRPPEGIPPAEVEWLRNEDLVDPSLDPNVYITREHSLVVRQARLADTANYTCVAKNIVAR
RRSASAAVIVYVDGSWSPWSKWSACGLDCTHWRSRECSDPAPRNGGEECQGTDLDTRNCTSDLCVHSA
SGPEDVALYVGLIAVAVCLVLLLLVLILVYCRKKEGLDSDVADSSILTSGFQPVSIKPSKADN

| | | |
|---|---|---|
| NOV21e, CG53908-04 | SEQ ID NO: 513 | 2752 bp |

| | | |
|---|---|---|
| DNA Sequence | ORF Start: ATG at 46 | ORF Stop: TGA at 2740 |

CCGCGGGGCCCCGCGCCCGGCCCGCCCGCCTGCCCGCCCGCGGCCATGGCCGTCCGGCCCGGCCTGTG
GCCAGCGCTCCTGGGCATAGTCCTCGCCGCTTGGCTCCGCGGCTCGGGTGCCCAGCAGAGTGCCACCG
TGGCCAACCCAGTGCCTGGTGCCAACCCGGACCTGCTTCCCCACTTCCTGGTGGAGCCCGAGGATGTG
TACATCGTCAAGAACAAGCCAGTGCTGCTTGTGTGCAAGGCCGTGCCCGCCACGCAGATCTTCTTCAA
GTGCAACGGGGAGTGGGTGCGCCAGGTGGACCACGTGATCGAGCGCAGCACAGACGGGAGCAGTGGGC
TGCCCACCATGGAGGTCCGCATTAATGTCTCAAGGCAGCAGGTCGAGAAGGTGTTCGGGCTGGAGGAA
TACTGGTGCCAGTGCGTGGCATGGAGCTCCTCGGGCACCACCAAGAGTCAGAAGGCCTACATCCGCAT
AGCCAGATTGCGCAAGAACTTCGAGCAGGAGCCGCTGGCCAAGGAGGTGTCCCTGGAGCAGGGCATCG
TGCTGCCCTGCCGTCCACCGGAGGGCATCCCTCCAGCCGAGGTGGAGTGGCTCCGGAACGAGGACCTG
GTGGACCCGTCCCTGGACCCCAATGTATACATCACGCGGGAGCACAGCCTGGTGGTGCGACAGGCCCG
CCTTGCTGACACGGCCAACTACACCTGCGTGGCCAAGAACATCGTGGCACGTCGCCGCAGCGCCTCCG
CTGCTGTCATCGTCTACGTGAACGGTGGGTGGTCGACGTGGACCGAGTGGTCCGTCTGCAGCGCCAGC
TGTGGGCGCGGCTGGCAGAAACGGAGCCGGAGCTGCACCAACCCGGCGCCTCTCAACGGGGGCGCTTT
CTGTGAGGGGCAGAATGTCCAGAAAACAGCCTGCGCCACCCTGTGCCCAGTAGACGGCAGCTGGAGCC
CGTGGAGCAAGTGGTCGGCCTGTGGGCTGGACTGCACCCACTGGCGGAGCCGTGAGTGCTCTGACCCA
GCACCCCGCAACGGAGGGGAGGAGTGCCAGGGCACTGACCTGGACACCCGCAACTGTACCAGTGACCT
CTGTGTACACAGTGCTTCTGGCCCTGAGGACGTGGCCCTCTATGTGGGCCTCATCGCCGTGGCCGTCT
GCCTGGTCCTGCTGCTGCTTGTCCTCATCCTCGTTTATTGCCGGAAGAAGGAGGGGCTGGACTCAGAT
GTGGCTGACTCGTCCATTCTCACCTCAGGCTTCCAGCCCGTCAGCATCAAGCCCAGCAAGCAGACAAA
CCCCCATCTGCTCACCATCCAGCCGGACCTCAGCACCACCACCACCACCTACCAGGGCAGTCTCTGTC
CCCGGCAGGATGGGCCCAGCCCCAAGTTCCAGCTCACCAATGGGCACCTGCTCAGCCCCCTGGGTGGC
GGCCGCCACACACTGCACCACAGCTCTCCCACCTCTGAGGCCGAGGAGTTCGTCTCCCGCCTCTCCAC
CCAGAACTACTTCCGCTCCCTGCCCCGAGGCACCAGCAACATGACCTATGGGACCTTCAACTTCCTCG
GGGGCCGGCTGATGATCCCTAATACAGGTATCAGCCTCCTCATCCCCCCAGATGCCATACCCCGAGGG
AAGATCTATGAGATCTACCTCACGCTGCACAAGCCGGAAGACGTGAGGTTGCCCCTAGCTGGCTGTCA
CTATGGACCACTGTGGGGAGCCCAGCCCTGACAGCTGGAGCCTGCGCCTCAAAAAGCAGTCGTGCGAG
GGCAGCTGGGAGGATGTGCTGCACCTGGGCGAGGAGGCGCCCTCCCACCTCTACTACTGCCAGCTGGA
GGCCAGTGCCTGCTACGTCTTCACCGAGCAGCTGGGCCGCTTTGCCCTGGTGGGAGAGGCCCTCAGCG
TGGCTGCCGCCAAGCGCCTCAAGCTGCTTCTGTTTGCGCCGGTGGCCTGCACCTCCCTCGAGTACAAC
ATCCGGGTCTACTGCCTGCATGACACCCACGATGCACTCAAGGAGGTGGTGCAGCTGGAGAAGCAGCT
GGGGGGACAGCTGATCCAGGAGCCACGGGTCCTGCACTTCAAGGACAGTTACCACAACCTGCGCCTAT
CCATCCACGATGTGCCCAGCTCCCTGTGGAAGAGTAAGCTCCTTGTCAGCTACCAGGAGATCCCCTTT
TATCACATCTGGAATGGCACGCAGCGGTACTTGCACTGCACCTTCACCCTGGAGCGTGTCAGCCCCAG
CACTAGTGACCTGGCCTGCAAGCTGTGGGTGTGGCAGGTGGAGGGCGACGGGCAGAGCTTCAGCATCA
ACTTCAACATCACCAAGGACACAAGGTTTGCTGAGCTGCTGGCTCTGGAGAGTGAAGCGGGGGTCCCA
GCCCTGGTGGGCCCCAGTGCCTTCAAGATCCCCTTCCTCATTCGGCAGAAGATAATTTCCAGCCTGGA
CCCACCCTGTAGGCGGGGTGCCGACTGGCGGACTCTGGCCCAGAAACTCCACCTGGACAGCCATCTCA
GCTTCTTTGCCTCCAAGCCCAGCCCCACAGCCATGATCCTCAACCTGTGGGAGGCGCGGCACTTCCCC
AACGGCAACCTCAGCCAGCTGGCTGCAGCAGTGGCTGGACTGGGCCAGCCAGACGCTGGCCTCTTCAC
AGTGTCGGAGGCTGAGTGCTGAGGCCGGCCAG

NOV21e, CG53908-04

| | | | |
|---|---|---|---|
| Protein Sequence | SEQ ID NO: 514 | 898 aa | MW at 98841.9kD |

MAVRPGLWPALLGIVLAAWLRGSGAQQSATVANPVPGANPDLLPHFLVEPEDVYIVKNKPVLLVCKAV
PATQIFFKCNGEWVRQVDHVIERSTDGSSGLPTMEVRINVSRQQVEKVFGLEEYWCQCVAWSSSGTTK
SQKAYIRIARLRKNFEQEPLAKEVSLEQGIVLPCRPPEGIPPAEVEWLRNEDLVDPSLDPNVYITREH
SLVVRQARLADTANYTCVAKNIVARRRSASAAVIVYVNGGWSTWTEWSVCSASCGRGWQKRSRSCTNP
APLNGGAFCEGQNVQKTACATLCPVDGSWSPWSKWSACGLDCTHWRSRECSDPAPRNGGEECQGTDLD
TRNCTSDLCVHSASGPEDVALYVGLIAVAVCLVLLLLVLILVYCRKKEGLDSDVADSSILTSGFQPVS
IKPSKADNPHLLTIQPDLSTTTTTYQGSLCPRQDGPSPKFQLTNGHLLSPLGGGRHTLHHSSPTSEAE
EFVSRLSTQNYFRSLPRGTSNMTYGTFNFLGGRLMIPNTGISLLIPPDAIPRGKIYEIYLTLHKPEDV
RLPLAGCQTLLSPIVSCGPPGVLLTRPVILAMDHCGEPSPDSWSLRLKKQSCEGSWEDVLHLGEEAPS
HLYYCQLEASACYVFTEQLGRFALVGEALSVAAAKRLKLLLFAPVACTSLEYNIRVYCLHDTHDALKE
VVQLEKQLGGQLIQEPRVLHFKDSYHNLRLSIHDVPSSLWKSKLLVSYQEIPFYHIWNGTQRYLHCTF
TLERVSPSTSDLACKLWVWQVEGDGQSFSINFNITKDTRFAELLALESEAGVPALVGPSAFKIPFLIR
QKIISSLDPPCRRGADWRTLAQKLHLDSHLSFFASKPSPTAMILNLWEARHFPNGNLSQLAAAVAGLG
QPDAGLFTVSEAEC

| | | |
|---|---|---|
| NOV21f, CG53908-05 | SEQ ID NO: 515 | 1002 bp |

| | | |
|---|---|---|
| DNA Sequence | ORF Start: at 7 | ORF Stop: at 997 |

GGATCCCAGCAGAGTGCCACCGTGGCCAACCCAGTGCCTGGTGCCAACCCGGACCTGCTTCCCCACTT
CCTGGTGGAGCCCGAGGATGTGTACATCGTCAAGAACAAGCCAGTGCTGCTTGTGTGCAAGGCCGTGC
CCGCCACGCAGATCTTCTTCAAGTGCAACGGGGAGTGGGTGCGCCAGGTGGACCACGTGATCGAGCGC
AGCACAGACGGGAGCAGTGGGCTGCCCACCATGGAGGTCCGCATTAATGTCTCAAGGCAGCAGGTCGA
GAAGGTGTTCGGGCTGGAGGAATACTGGTGCCAGTGCGTGGCATGGAGCTCCTCGGGCACCACCAAGA
GTCAGAAGGCCTACATCCGCATAGCCTATTTGCGCAAGAACTTCGAGCAGGAGCCACTGGCCAAGGAG
GTGTCCCTGGAGCAGGGCATCGTGCTGCCCTGCCGTCCACCGGAGGGCATCCCTCCAGCCGAGGTGGA
GTGGCTCCGGAACGAGGACCTGGTGGACCCGTCCCTGGACCCCAATGTATACATCACGCGGGAGCACA
GCCTGGTGGTGCGACAGGCCCGCCTTGCTGACACGGCCAACTACACCTGCGTGGCCAAGAACATCGTG
GCACGTCGCCGCAGCGCCTCCGCTGCTGTCATCGTCTACGTGAACGGTGGGTGGTCGACGTGGACCGA
GTGGTCCGTCTGCAGCGCCAGCTGTGGGCGCGGCTGGCAGAAACGGAGCCGGAGCTGCACCAACCCGG
CGCCTCTCAACGGGGGCGCTTTCTGTGAGGGGCAGAATGTCCAGAAAACAGCCTGCGCCACCCTGTGC
CCAGTGGACGGCAGCTGGAGCCCGTGGAGCAAGTGGTCGGCCTGTGGGCTGGACTGCACCCACTGGCG
GAGCCGTGAGTGCTCTGACCCAGCACCCCGCAACGGAGGGGAGGAGTGCCAGGGCACTGACCTGGACA
CCCGCAACTGTACCAGTGACCTCTGTGTACACAGTGCTTCTGGCCTCGAG

NOV21f, CG53908-05

| | | | |
|---|---|---|---|
| Protein Sequence | SEQ ID NO: 516 | 330 aa | MW at 36417.7kD |

QQSATVANPVPGANPDLLPHFLVEPEDVYIVKNKPVLLVCKAVPATQIFFKCNGEWVRQVDHVIERST
DGSSGLPTMEVRINVSRQQVEKVFGLEEYWCQCVAWSSSGTTKSQKAYIRIAYLRKNFEQEPLAKEVS
LEQGIVLPCRPPEGIPPAEVEWLRNEDLVDPSLDPNVYITREHSLVVRQARLADTANYTCVAKNIVAR
RRSASAAVIVYVNGGWSTWTEWSVCSASCGRGWQKRSRSCTNPAPLNGGAFCEGQNVQKTACATLCPV
DGSWSPWSKWSACGLDCTHWRSRECSDPAPRNGGEECQGTDLDTRNCTSDLCVHSASG

| | | |
|---|---|---|
| NOV21g, CG53908-06 | SEQ ID NO: 517 | 2575 bp |

| | | |
|---|---|---|
| DNA Sequence | ORF Start: at 3 | ORF Stop: TGA at 2502 |

CAGCGCTCCTGGGCATAGTCCTCGCCGCTTGGCTCCGCGGCTCGGGTGCCCAGCAGAGTGCCACCGTG
GCCAACCCAGTGCCTGGTGCCAACCCGGACCTGCTTCCCCACTTCCTGGTGGAGCCCGAGGATGTGTA
CATCGTCAAGAACAAGCCAGTGCTGCTTGTGTGCAAGGCCGTGCCCGCCACGCAGATCTTCTTCAAGT
GCAACGGGGAGTGGGTGCGCCAGGTGGACCACGTGATCGAGCGCAGCACAGACGGGAGCAGTGGGCTG
CCCACCATGGAGGTCCGCATTAATGTCTCAAGGCAGCAGGTCGAGAAGGTGTTCGGGCTGGAGGAATA
CTGGTGCCAGTGCGTGGCATGGAGCTCCTCGGGCACCACCAAGAGTCAGAAGGCCTACATCCGCATAG
CCTATTTGCGCAAGAACTTCGAGCAGGAGCCGCTGGCCAAGGAGGTGTCCCTGGAGCAGGGCATCGTG
CTGCCCTGCCGTCCACCGGAGGGCATCCCTCCAGCCGAGGTGGAGTGGCTCCGGAACGAGGACCTGGT
GGACCCGTCCCTGGACCCCAATGTATACATCACGCGGGAGCACAGCCTGGTGGTGCGACAGGCCCGCC
TTGCTGACACGGCCAACTACACCTGCGTGGCCAAGAACATCGTGGCACGTCGCCGCAGCGCCTCCGCT
GCTGTCATCGTCTACGTGGACGGCAGCTGGAGCCCGTGGAGCAAGTGGTCGGCCTGTGGGCTGGACTG
CACCCACTGGCGGAGCCGTGAGTGCTCTGACCCAGCACCCCGCAACGGAGGGGAGGAGTGCCAGGGCA
CTGACCTGGACACCCGCAACTGTACCAGTGACCTCTGTGTACACACTGCTTCTGGCCCTGAGGACGTG
GCCCTCTATGTGGGCCTCATCGCCGTGGCCGTCTGCCTGGTCCTGCTGCTGCTTGTCCTCATCCTCGT
TTATTGCCGGAAGAAGGAGGGGCTGGACTCAGATGTGGCTGACTCGTCCATTCTCACCTCAGGCTTCC
ACCACCACCACCACCTACCAGGGCAGTCTCTGTCCCCGGCAGGATGGGCCCAGCCCCAAGTTCCAGCT
CACCAATGGGCACCTGCTCAGCCCCCTGGGTGGCGGCCGCCACACACTGCACCACAGCTCTCCCACCT
CTGAGGCCGAGGAGTTCGTCTCCCGCCTCTCCACCCAGAACTACTTCCGCTCCCTGCCCCGAGGCACC
AGCAACATGACCTATGGGACCTTCAACTTCCTCGGGGGCCGGCTGATGATCCCTAATACAGGAATCAG
CCTCCTCATCCCCCCAGATGCCATACCCCGAGGGAAGATCTATGAGATCTACCTCACGCTGCACAAGC
CGGAAGACGTGAGGTTGCCCCTAGCTGGCTGTCAGACCCTGCTGAGTCCCATCGTTAGCTGTGGACCC
CCTGGCGTCCTGCTCACCCGGCCAGTCATCCTGGCTATGGACCACTGTGGGGAGCCCAGCCCTGACAG
CTGGAGCCTGCGCCTCAAAAAGCAGTCGTGCGAGGGCAGCTGGGAGGATGTGCTGCACCTGGGCGAGG
AGGCGCCCTCCCACCTCTACTACTGCCAGCTGGAGGCCAGTGCCTGCTACGTCTTCACCGAGCAGCTG
GGCCGCTTTGCCCTGGTGGGAGAGGCCCTCAGCGTGGCTGCCGCCAAGCGCCTCAAGCTGCTTCTGTT
TGCGCCGGTGGCCTGCACCTCCCTCGAGTACAACATCCGGGTCTACTGCCTGCATGACACCCACGATG
CACTCAAGGAGGTGGTGCAGCTGGAGAAGCAGCTGGGGGGACAGCTGATCCAGGAGCCACGGGTCCTG
CACTTCAAGGACAGTTACCACAACCTGCGCCTATCCATCCACGATGTGCCCAGCTCCCTGTGGAAGAG
TAAGCTCCTTGTCAGCTACCAGGAGATCCCCTTTTATCACATCTGGAATGGCACGCAGCGGTACTTGC
ACTGCACCTTCACCCTGGAGCGTGTCAGCCCCAGCACTAGTGACCTGGCCTGCAAGCTGTGGGTGTGG
CAGGTGGAGGGCGACGGGCAGAGCTTCAGCATCAACTTCAACATCACCAAGGACACAAGGTTTGCTGA
GCTGCTGGCTCTGGAGAGTGAAGCGGGGGTCCCAGCCCTGGTGGGCCCCAGTGCCTTCAAGATCCCCT
TCCTCATTCGGCAGAAGATAATTTCCAGCCTGGACCCACCCTGTAGGCGGGGTGCCGACTGGCGGACT
CTGGCCCAGAAACTCCACCTGGACAGCCATCTCAGCTTCTTTGCCTCCAAGCCCAGCCCCACAGCCAT
GATCCTCAACCTGTGGGAGGCGCGGCACTTCCCCAACGGCAACCTCAGCCAGCTGGCTGCAGCAGTGG
CTGGACTGGGCCAGCCAGACGCTGGCCTCTTCACAGTGTCGGAGGCTGAGTGCTGAGGCCGGCCAGGC
CCGACACCTACACTCTCACCAGCTTTGGCACCCACCAAGGACAGGCAGAAGCCGGACAG

NOV21g, CG53908-06

| | | | |
|---|---|---|---|
| Protein Sequence | SEQ ID NO: 518 | 833 aa | MW at 91922.0kD |

ALLGIVLAAWLRGSGAQQSATVANPVPGANPDLLPHFLVEPEDVYIVKNKPVLLVCKAVPATQIFFKC
NGEWVRQVDHVTERSTDGSSGLPTMEVRINVSRQQVEKVFGLEEYWCQCVAWSSSGTTKSQKAYIRIA
YLRKNFEQEPLAKEVSLEQIVLPCRPPEGIPPAEVEWLRNEDLVDPSLDPNVYITREHSLVVRQARL
ADTANYTCVAKNIVARRRSASAAVIVYVDGSWSPWSKWSACGLDCTHWRSRECSDPAPRNGGEECQGT
DLDTRNCTSDLCVHTASGPEDVALYVGLIAVAVCLVLLLLVLILVYCRKKEGLDSDVADSSILTSGFQ
PVSIKPSKADNPHLLTIQPDLSTTTTTYQGSLCPRQDGPSPKFQLTNGHLLSPLGGGRHTLHHSSPTS
EAEEFVSRLSTQNYFRSLPRGTSNMTYGTFNFLGGRLMIPNTGISLLIPPDAIPRGKIYEIYLTLHKP
EDVRLPLAGCQTLLSPIVSCGPPGVLLTRPVILAMDHCGEPSPDSWSLRLKKQSCEGSWEDVLHLGEE
APSHLYYCQLEASACYVFTEQLGRFALVGEALSVAAAKRLKLLLFAPVACTSLEYNIRVYCLHDTHDA
LKEVVQLEKQLGGQLIQEPRVLHFKDSYHNLRLSIHDVPSSLWKSKLLVSYQEIPFYHIWNGTQRYLH
CTFTLERVSPSTSDLACKLWVWQVEGDGQSFSINFNITKDTRFAELLALESEAGVPALVGPSAFKIPF
LIRQKIISSLDPPCRRGADWRTLAQKLHLDSHLSFFASKPSPTAMILNLWEARHFPNGNLSQLAAAVA
GLGQPDAGLFTVSEAEC

| | | |
|---|---|---|
| NOV21h, CG53908-07 | SEQ ID NO: 519 | 2463 bp |

| | | |
|---|---|---|
| DNA Sequence | ORF Start: at 7 | ORF Stop: at 2458 |

GGATCCCAGCAGAGTGCCACCGTGGCCAACCCAGTGCCTGGTGCCAACCCGGACCTGCTTCCCCACTT
CCTGGTGGAGCCCGAGGATGTGTACATCGTCAAGAACAAGCCAGTGCTGCTTGTGTGCAAGGCCGTGC
CCGCCACGCAGATCTTCTTCAAGTGCAACGGGGAGTGGGTGCGCCAGGTGGACCACGTGATCGAGCGC
AGCACAGACGGGAGCAGTGGGCTGCCCACCATGGAGGTCCGCATTAATGTCTCAAGGCAGCAGGTCGA
GAAGGTGTTCGGGCTGGAGGAATACTGGTGCCAGTGCGTGGCATGGAGCTCCTCGGGCACCACCAAGA
GTCAGAAGGCCTACATCCGCATAGCCTATTTGCGCAAGAACTTCGAGCAGGAGCCGCTGGCCAAGGAG
GTGTCCCTGGAGCAGGGCATCGTGCTGCCCTGCCGTCCACCGGAGGGCATCCCTCCAGCCGAGGTGGA
GTGGCTCCGGAACGAGGACCTGGTGGACCCGTCCCTGGACCCCAATGTATACATCACGCGGGAGCACA
GCACGTCGCCGCAGCGCCTCCGCTGCTGTCATCGTCTACGTGGACGGCAGCTGGAGCCCGTGGAGCAA
GTGGTCGGCCTGTGGGCTGGGCTGCACCCACTGGCGGAGCCGTGAGTGCTCTGACCCAGCACCCCGCA
ACGGAGGGGAGGAGTGCCAGGGCACTGACCTGGACACCCGCAACTGTACCAGTGACCTCTGTGTACAC
ACTGCTTCTGGCCCTGAGGACGTGGCCCTCTATGTGGGCCTCATCGCCGTGGCCGTCTGCCTGGTCCT
GCTGCTGCTTGTCCTCATCCTCGTTTATTGCCGGAAGAAGGAGGGGCTGGACTCAGATGTGGCTGACT
CGTCCATTCTCACCTCAGGCTTCCAGCCCGTCAGCATCAAGCCCAGCAAAGCAGACAACCCCCATCTG
CTCACCATCCAGCCGGACCTCAGCACCACCACCACCACCTACCAGGGCAGTCTCTGTCCCCGGCAGGA
TGGGCCCAGCCCCAAGTTCCAGCTCACCAATGGGCACCTGCTCAGCCCCCTGGGTGGCGGCCGCCACA
CACTGCACCACAGCTCTCCCACCTCTGAGGCCGAGGAGTTCGTCTCCCGCCTCTCCACCCAGAACTAC
TTCCGCTCCCTGCCCCGAGGCACCAGCAACATGACCTATGGGACCTTCAACTTCCTCGGGGGCCGGCT
GATGATCCCTAATACAGGAATCAGCCTCCTCATCCCCCCAGATGCCATACCCCGAGGGAAGATCTATG
AGATCTACCTCACGCTGCACAAGCCGGAAGACGTGAGGTTGCCCCTAGCTGGCTGTCAGACCCTGCTG
AGTCCCATCGTTAGCTGTGGACCCCCTGGCGTCCTGCTCACCCGGCCAGTCATCCTGGCTATGGACCA
AGGATGTGCTGCACCTGGGCGAGGAGGCGCCCTCCCACCTCTACTACTGCCAGCTGGAGGCCAGTGCC
TGCTACGTCTTCACCGAGCAGCTGGGCCGCTTTGCCCTGGTGGGAGAGGCCCTCAGCGTGGCTGCCGC
CAAGCGCCTCAAGCTGCTTCTGTTTGCGCCGGTGGCCTGCACCTCCCTCGAGTACAACATCCGGGTCT
CTGATCCAGGAGCCACGGGTCCTGCACTTCAAGGACAGTTACCACAACCTGCGCCTATCCATCCACGA
TGTGCCCAGCTCCCTGTGGAAGAGTAAGCTCCTTGTCAGCTACCAGGAGATCCCCTTTTATCACATCT
GGAATGGCACGCAGCGGTACTTGCACTGCACCTTCACCCTGGAGCGTGTCAGCCCCAGCACTAGTGAC
CTGGCCTGCAAGCTGTGGGTGTGGCAGGTGGAGGGCGACGGGCAGAGCTTCAGCATCAACTTCAACAT
CACCAAGGACACAAGGTTTGCTGAGCTGCTGGCTCTGGAGAGTGAAGCGGGGGTCCCAGCCCTGGTGG
GCCCCAGTGCCTTCAAGATCCCCTTCCTCATTCGGCAGAAGATAATTTCCAGCCTGGACCCACCCTGT
AGGCGGGGTGCCGACTGGCGGACTCTGGCCCAGAAACTCCACCTGGACAGCCATCTCAGCTTCTTTGC
GCTGAGTGCAAGCTT

NOV21h, CG53908-07

| | | | |
|---|---|---|---|
| Protein Sequence | SEQ ID NO: 520 | 817 aa | MW at 90314.2kD |

QQSATVANPVPGANPDLLPHFLVEPEDVYIVKNKPVLLVCKAVPATQIFFKCNGEWVRQVDHVIERST
DGSSGLPTMEVRINVSRQQVEKVFGLEEYWCQCVAWSSSGTTKSQKAYIRIAYLRKNFEQEPLAKEVS
LEQGIVLPCRPPEGIPPAEVEWLRNEDLVDPSLDPNVYITREHSLVVRQARLADTANYTCVAKNIVAR
RRSASAAVIVYVDGSWSPWSKWSACGLGCTHWRSRECSDPAPRNGGEECQGTDLDTRNCTSDLCVHTA
SGPEDVALYVGLIAVAVCLVLLLLVLILVYCRKKEGLDSDVADSSILTSGFQPVSIKPSKADNPHLLT
IQPDLSTTTTTYQGSLCPRQDGPSPKFQLTNGHLLSPLGGGRHTLHHSSPTSEAEEFVSRLSTQNYFR
SLPRGTSNMTYGTFNFLGGRLMIPNTGISLLIPPDAIPRGKIYEIYLTLHKPEDVRLPLAGCQTLLSP
IVSCGPPGVLLTRPVILAMDHCGEPSPDSWSLRLKKQSCEGSWEDVLHLGEEAPSHLYYCQLEASACY
VFTEQLGRFALVGEALSVAAAKRLKLLLFAPVACTSLEYNIRVYCLHDTHDALKEVVQLEKQLGGQLI
QEPRVLHFKDSYHNLRLSIHDVPSSLWKSKLLVSYQEIPFYHIWNGTQRYLHCTFTLERVSPSTSDLA
CKLWVWQVEGDGQSFSINFNITKDTRFAELLALESEAGVPALVGPSAFKIPFLIRQKIISSLDPPCRR
GADWRTLAQKLHLDSHLSFFASKPSPTAMILNLWEARHFPNGNLSQLAAAVAGLGQPDAGLFTVSEAE
C

| | |
|---|---|
| SEQ ID NO: 521 | 2881 bp |

| | | |
|---|---|---|
| NOV21i, SNP13382444 of | ORF Start: at 423 | ORF Stop: end of sequence |

| | | |
|---|---|---|
| CG53908-01, DNA Sequence | SNP Pos: 420 | SNP Change: C to T |

AGCTGGGGCTCCGGGCTGAGGCGCTAAAGCCGCCCTCCCGCCCGCGGGGCCCCGCGCCCGGCCCGCCC
GCCTGCCCGCCCGCGGCCATGGCCGTCCGGCCCGGCCTGTGGCCAGCGCTCCTGGGCATAGTCCTCGC
CGCTTGGCTCCGCGGCTCGGGTGCCCAGCAGAGTGCCACCGTGGCCAACCCAGTGCCTGGTGCCAACC
CGGACCTGCTTCCCCACTTCCTGGTGGAGCCCGAGGATGTGTACATCGTCAAGAACAAGCCAGTGCTG
CTTGTGTGCAAGGCCGTGCCCGCCACGCAGATCTTCTTCAAGTGCAACGGGGAGTGGGTGCGCCAGGT
GGACCACGTGATCGAGCGCAGCACAGACGGGAGCAGTGGTGAGCCGACCATGGAGGTCCGCATTAATG
TCTCAAGGCAGTAGGTCGAGAAGGTGTTCGGGCTGGAGGAATACTGGTGCCAGTGCGTGGCATGGAGC
TCCTCGGGCACCACCAAGAGTCAGAAGGCCTACATCCGCATAGCCAGATTGCGCAAGAACTTCGAGCA
GGAGCCGCTGGCCAAGGAGGTGTCCCTGGAGCAGGGCATCGTGCTGCCCTGCCGTCCACCGGAGGGCA
TCCCTCCAGCCGAGGTGGAGTGGCTCCGGAACGAGGACCTGGTGGACCCGTCCCTGGACCCCAATGTA
TACATCACGCGGGAGCACAGCCTGGTGGTGCGACAGGCCCGCCTTGCTGACACGGCCAACTACACCTG
CGTGGCCAAGAACATCGTGGCACGTCGCCGCAGCGCCTCCGCTGCTGTCATCGTCTACGTGAACGGTG
GGTGGTCGACGTGGACCGAGTGGTCCGTCTGCAGCGCCAGCTGTGGGCGCGGCTGGCAGAAACGGAGC
CACCGTCTCCTCTCTGCTTGTCTCTGTGGACGGCAGCTGGAGCCCGTGGAGCAAGTGGTCGGCCTGTG
GGCTGGACTGCACCCACTGGCGGAGCCGTGAGTGCTCTGACCCAGCACCCCGCAACGGAGGGGAGGAG
TGAGGACGTGGCCCTCTATGTGGGCCTCATCGCCGTGGCCGTCTGCCTGGTCCTGCTGCTGCTTGTCC
TCATCCTCGTTTATTGCCGGAAGAAGGAGGGGCTGGACTCAGATGTGGCTGACTCGTCCATTCTCACC
TCAGGCTTCCAGCCCGTCAGCATCAAGCCCAGCAAAGCAGACAACCCCCATCTGCTCACCATCCAGCC
GGACCTCAGCACCACCACCACCTACCAGGGCAGTCTCTGTCCCCGGCAGGATGGGCCCAGCCCCAAGT
TCCAGCTCACCAATGGGCACCTGCTCAGCCCCCTGGGTGGCGGCCGCCACACACTGCACCACAGCTCT
CCCACCTCTGAGGCCGAGGAGTTCGTCTCCCGCCTCTCCACCCAGAACTACTTCCGCTCCCTGCCCCG
AGGCACCAGCAACATGACCTATGGGACCTTCAACTTCCTCGGGGGCCGGCTGATGATCCCTAATACAG
GTATCAGCCTCCTCATCCCCCCAGATGCCATACCCCGAGGGAAGATCTATGAGATCTACCTCACGCTG
CACAAGCCGGAAGACGTGAGGTTGCCCCTAGCTGGCTGTCAGACCCTGCTGAGTCCCATCGTTAGCTG
CTGACAGCTGGAGCCTGCGCCTCAAAAAGCAGTCGTGCGAGGGCAGCTGGGAGCAGGATGTGCTGCAC
CTGGGCGAGGAGGCGCCCTCCCACCTCTACTACTGCCAGCTGGAGGCCAGTGCCTGCTACGTCTTCAC
CGAGCAGCTGGGCCGCTTTGCCCTGGTGGGAGAGGCCCTCAGCGTGGCTGCCGCCAAGCGCCTCAAGC
TGCTTCTGTTTGCGCCGGTGGCCTGCACCTCCCTCGAGTACAACATCCGGGTCTACTGCCTGCATGAC
ACCCACGATGCACTCAAGGAGGTGGTGCAGCTGGAGAAGCAGCTGGGGGGACAGCTGATCCAGGAGCC
ACGGGTCCTGCACTTCAAGGACAGTTACCACAACCTGCGCCTATCCATCCACGATGTGCCCAGCTCCC
TGTGGAAGAGTAAGCTCCTTGTCAGCTACCAGGAGATCCCCTTTTATCACATCTGGAATGGCACGCAG
GTGGGTGTGGCAGGTGGAGGGCGACGGGCAGAGCTTCAGCATCAACTTCAACATCACCAAGGACACAA
GGTTTGCTGAGCTGCTGGCTCTGGAGAGTGAAGCGGGGGTCCCAGCCCTGGTGGGCCCCAGTGCCTTC
AAGATCCCCTTCCTCATTCGGCAGAAGATAATTTCCAGCCTGGACCCACCCTGTAGGCGGGGTGCCGA
CTGGCGGACTCTGGCCCAGAAACTCCACCTGGACAGCCATCTCAGCTTCTTTGCCTCCAAGCCCAGCC
CCACAGCCATGATCCTCAACCTGTGGGAGGCGCGGCACTTCCCCAACGGCAACCTCAGCCAGCTGGCT
GCAGCAGTGGCTGGACTGGGCCAGCCAGACGCTGGCCTCTTCACAGTGTCGGAGGCTGAGTGCTGAGG
CCGGCCAGGCCCGACACCTACACTCTCACCAGCTTTGGCACCCACCAAGGACAGGCAGAAGCCGGACA
GGGGCCCTTCCCCACACCGGGGAGA

| | | |
|---|---|---|
| NOV21i, SNP13382444 of | SEQ ID NO: 522 | MW at 98934.8kD |

| | | | |
|---|---|---|---|
| CG53908-01, Protein Sequence | SNP Pos: 112 | 898 aa | SNP Change: Gln to End |

MAVRPGLWPALLGIVLAAWLRGSGAQQSATVANPVPGANPDLLPHFLVEPEDVYIVKNKPVLLVCKAV
PATQIFFKCNGEWVRQVDHVIERSTDGSSGEPTMEVRINVSRQVEKVFGLEEYWCQCVAWSSSGTTKS
QKAYIRIARLRKNFEQEPLAKEVSLEQGIVLPCRPPEGIPPAEVEWLRNEDLVDPSLDPNVYITREHS
LVVRQARLADTANYTCVAKNIVARRRSASAAVIVYVNGGWSTWTEWSVCSASCGRGWQKRSRSCTNPA
PLNGGAFCEGQNVHDRTVSSLLVSVDGSWSPWSKWSACGLDCTHWRSRECSDPAPRNGGEECQGTDLD
TRNCTSDLCVHSASGPEDVALYVGLIAVAVCLVLLLLVLILVYCRKKEGLDSDVADSSILTSGFQPVS
IKPSKADNPHLLTIQPDLSTTTTYQGSLCPRQDGPSPKFQLTNGHLLSPLGGGRHTLHHSSPTSEAEE
FVSRLSTQNYFRSLPRGTSNMTYGTFNFLGGRLMIPNTGISLLIPPDAIPRGKIYEIYLTLHKPEDVR
LPLAGCQTLLSPIVSCGPPGVLLTRPVILAMDHCGEPSPDSWSLRLKKQSCEGSWEQDVLHLGEEAPS
HLYYCQLEASACYVFTEQLGRFALVGEALSVAAAKRLKLLLFAPVACTSLEYNIRVYCLHDTHDALKE
VVQLEKQLGGQLIQEPRVLHFKDSYHNLRLSIHDVPSSLWKSKLLVSYQEIPEYNIWNGTQRYLHCTF
TLERVSPSTSDLACKLWVWQVEGDGQSFSINFNITKDTRFAELLALESEAGVPALVGPSAFKIPELIR
QKIISSLDPPCRRGADWRTLAQKLHLDSHLSFFASKPSPTAMILNLWEARHFPNGNLSQLAAAVAGLG
QPDAGLFTVSEAEC

| | |
|---|---|
| SEQ ID NO: 523 | 2881 bp |

| | | |
|---|---|---|
| NOV21j, SNP13375491 of | ORF Start: ATG at 87 | ORF Stop: TGA at 2784 |

| | | |
|---|---|---|
| CG53908-01, DNA Sequence | SNP Pos: 1139 | SNP Change: A to G |

AGCTGGGGCTCCGGGCTGAGGCGCTAAAGCCGCCCTCCCGCCCGCGGGGCCCCGCGCCCGGCCCGCCC
GCCTGCCCGCCCGCGGCCATGGCCGTCCGGCCCGGCCTGTGGCCAGCGCTCCTGGGCATAGTCCTCGC
CGCTTGGCTCCGCGGCTCGGGTGCCCAGCAGAGTGCCACCGTGGCCAACCCAGTGCCTGGTGCCAACC
CGGACCTGCTTCCCCACTTCCTGGTGGAGCCCGAGGATGTGTACATCGTCAAGAACAAGCCAGTGCTG
CTTGTGTGCAAGGCCGTGCCCGCCACGCAGATCTTCTTCAAGTGCAACGGGGAGTGGGTGCGCCAGGT
TCTCAAGGCAGCAGGTCGAGAAGGTGTTCGGGCTGGAGGAATACTGGTGCCAGTGCGTGGCATGGAGC
GGAGCCGCTGGCCAAGGAGGTGTCCCTGGAGCAGGGCATCGTGCTGCCCTGCCGTCCACCGGAGGGCA
TCCCTCCAGCCGAGGTGGAGTGGCTCCGGAACGAGGACCTGGTGGACCCGTCCCTGGACCCCAATGTA
CGTGGCCAAGAACATCGTGGCACGTCGCCGCAGCGCCTCCGCTGCTGTCATCGTCTACGTGAACGGTG
GGTGGTCGACGTGGACCGAGTGGTCCGTCTGCAGCGCCAGCTGTGGGCGCGGCTGGCAGAAACGGAGC
CGGAGCTGCACCAACCCGGCGCCTCTCAACGGGGGCGCTTTCTGTGAGGGGCAGAATGTCCATGACCG
CACCGTCTCCTCTCTGCTTGTCTCTGTGGACGGCAGCTGGAGCCCGTGGAGCAAGTGGTCGGCCTGTG
GGCTGGACTGCACCCACTGGCGGAGCCGTGAGTGCTCTGACCCAGCACCCCGCAACGGAGGGGAGGAG
TGCCAGGGCACTGACCTGGACACCCGCAACTGTACCAGTGACCTCTGTGTGCACAGTGCTTCTGGCCC
TGAGGACGTGGCCCTCTATGTGGGCCTCATCGCCGTGGCCGTCTGCCTGGTCCTGCTGCTGCTTGTCC
TCATCCTCGTTTATTGCCGGAAGAAGGAGGGGCTGGACTCAGATGTGGCTGACTCGTCCATTCTCACC
TCAGGCTTCCAGCCCGTCAGCATCAAGCCCAGCAAAGCAGACAACCCCCATCTGCTCACCATCCAGCC
GGACCTCAGCACCACCACCACCTACCAGGGCAGTCTCTGTCCCCGGCAGGATGGGCCCAGCCCCAAGT
TCCAGCTCACCAATGGGCACCTGCTCAGCCCCCTGGGTGGCGGCCGCCACACACTGCACCACAGCTCT
CCCACCTCTGAGGCCGAGGAGTTCGTCTCCCGCCTCTCCACCCAGAACTACTTCCGCTCCCTGCCCCG
AGGCACCAGCAACATGACCTATGGGACCTTCAACTTCCTCGGGGGCCGGCTGATGATCCCTAATACAG
GTATCAGCCTCCTCATCCCCCCAGATGCCATACCCCGAGGGAAGATCTATGAGATCTACCTCACGCTG
CACAAGCCGGAAGACGTGAGGTTGCCCCTAGCTGGCTGTCAGACCCTGCTGAGTCCCATCGTTAGCTG
TGGACCCCCTGGCGTCCTGCTCACCCGGCCAGTCATCCTGGCTATGGACCACTGTGGGGAGCCCAGCC
CTGACAGCTGGAGCCTGCGCCTCAAAAAGCAGTCGTGCGAGGGCAGCTGGGAGCAGGATGTGCTGCAC
CTGGGCGAGGAGGCGCCCTCCCACCTCTACTACTGCCAGCTGGAGGCCAGTGCCTGCTACGTCTTCAC
CGAGCAGCTGGGCCGCTTTGCCCTGGTGGGAGAGGCCCTCAGCGTGGCTGCCGCCAAGCGCCTCAAGC
TGCTTCTGTTTGCGCCGGTGGCCTGCACCTCCCTCGAGTACAACATCCGGGTCTACTGCCTGCATGAC
ACCCACGATGCACTCAAGGAGGTGGTGCAGCTGGAGAAGCAGCTGGGGGGACAGCTGATCCAGGAGCC
ACGGGTCCTGCACTTCAAGGACAGTTACCACAACCTGCGCCTATCCATCCACGATGTGCCCAGCTCCC
TGTGGAAGAGTAAGCTCCTTGTCAGCTACCAGGAGATCCCCTTTTATCACATCTGGAATGGCACGCAG
CGGTACTTGCACTGCACCTTCACCCTGGAGCGTGTCAGCCCCAGCACTAGTGACCTGGCCTGCAAGCT
GGTTTGCTGAGCTGCTGGCTCTGGAGAGTGAAGCGGGGGTCCCAGCCCTGGTGGGCCCCAGTGCCTTC
AAGATCCCCTTCCTCATTCGGCAGAAGATAATTTCCAGCCTGGACCCACCCTGTAGGCGGGGTGCCGA
CTGGCGGACTCTGGCCCAGAAACTCCACCTGGACAGCCATCTCAGCTTCTTTGCCTCCAAGCCCAGCC
CCACAGCCATGATCCTCAACCTGTGGGAGGCGCGGCACTTCCCCAACGGCAACCTCAGCCAGCTGGCT
GCAGCAGTGGCTGGACTGGGCCAGCCAGACGCTGGCCTCTTCACAGTGTCGGAGGCTGAGTGCTGAGG
CCGGCCAGGCCCGACACCTACACTCTCACCAGCTTTGGCACCCACCAAGGACAGGCAGAAGCCGGACA
GGGGCCCTTCCCCACACCGGGGAGA

| | | |
|---|---|---|
| NOV21j, SNP13375491 of | SEQ ID NO: 524 | MW at 99063.0kD |

| | | | |
|---|---|---|---|
| CG53908-01, Protein Sequence | SNP Pos: 351 | 899 aa | SNP Change: Val to Val |

MAVRPGLWPALLGIVLAAWLRGSGAQQSATVANPVPGANPDLLPHFLVEPEDVYIVKNKPVLLVCKAV
PATQIFFKCNGEWVRQVDHVIERSTDGSSGEPTMEVRINVSRQQVEKVFGLEEYWCQCVAWSSSGTTK
SQKAYIRIARLRKNFEQEPLAKEVSLEQGIVLPCRPPEGIPPAEVEWLRNEDLVDPSLDPNVYITREH
SLVVRQARLADTANYTCVAKNIVARRRSASAAVIVYVNGGWSTWTEWSVCSASCGRGWQKRSRSCTNP
APLNGGAFCEGQNVHDRTVSSLLVSVDGSWSPWSKWSACGLDCTHWRSRECSDPAPRNGGEECQGTDL
DTRNCTSDLCVHSASGPEDVALYVGLIAVAVCLVLLLLVLILVYCRKKEGLDSDVADSSILTSGFQPV
SIKPSKADNPHLLTIQPDLSTTTTYQGSLCPRQDGPSPKFQLTNGHLLSPLGGGRHTLHHSSPTSEAE
EFVSRLSTQNYFRSLPRGTSNMTYGTFNFLGGRLMIPNTGISLLIPPDAIPRGKIYEIYLTLHKPEDV
RLPLAGCQTLLSPIVSCGPPGVLLTRPVILAMDHCGEPSPDSWSLRLKKQSCEGSWEQDVLHLGEEAP
SHLYYCQLEASACYVFTEQLGRFALVGEALSVAAAKRLKLLLFAPVACTSLEYNIRVYCLHDTHDALK
EVVQLEKQLGGQLIQEPRVLHFKDSYHNLRISIHDVPSSLWKSKLLVSYQEIPFYHIWNGTQRYLHCT
FTLERVSPSTSDLACKLWVWQVEGDGQSFSINFNITKDTRFAELLALESEAGVPALVGPSAFKIPELI
RQKIISSLDPPCRRGADWRTLAQKLHLDSHLSFFASKPSPTAMILNLWEARHFPNGNLSQLAAAVAGL
GQPDAGLFTVSEAEC

| | |
|---|---|
| SEQ ID NO: 525 | 2881 bp |

| | | |
|---|---|---|
| NOV21k, SNP13375492 of | ORF Start: ATG at 87 | ORF Stop: TGA at 2784 |

| | | |
|---|---|---|
| CG53908-01, DNA Sequence | SNP Pos: 1225 | SNP Change: T to C |

AGCTGGGGCTCCGGGCTGAGGCGCTAAAGCCGCCCTCCCGCCCGCGGGGCCCCGCGCCCGGCCCGCCC
GCCTGCCCGCCCGCGGCCATGGCCGTCCGGCCCGGCCTGTGGCCAGCGCTCCTGGGCATAGTCCTCGC
CGCTTGGCTCCGCGGCTCGGGTGCCCAGCAGAGTGCCACCGTGGCCAACCCAGTGCCTGGTGCCAACC
CGGACCTGCTTCCCCACTTCCTGGTGGAGCCCGAGGATGTGTACATCGTCAAGAACAAGCCAGTGCTG
CTTGTGTGCAAGGCCGTGCCCGCCACGCAGATCTTCTTCAAGTGCAACGGGGAGTGGGTGCGCCAGGT
GGACCACGTGATCGAGCGCAGCACAGACGGGAGCAGTGGTGAGCCGACCATGGAGGTCCGCATTAATG
TCTCAAGGCAGCAGGTCGAGAAGGTGTTCGGGCTGGAGGAATACTGGTGCCAGTGCGTGGCATGGAGC
TCCTCGGGCACCACCAAGAGTCAGAAGGCCTACATCCGCATAGCCAGATTGCGCAAGAACTTCGAGCA
GGAGCCGCTGGCCAAGGAGGTGTCCCTGGAGCAGGGCATCGTGCTGCCCTGCCGTCCACCGGAGGGCA
TCCCTCCAGCCGAGGTGGAGTGGCTCCGGAACGAGGACCTGGTGGACCCGTCCCTGGACCCCAATGTA
TACATCACGCGGGAGCACAGCCTGGTGGTGCGACAGGCCCGCCTTGCTGACACGGCCAACTACACCTG
CGTGGCCAAGAACATCGTGGCACGTCGCCGCAGCGCCTCCGCTGCTGTCATCGTCTACGTGAACGGTG
GGTGGTCGACGTGGACCGAGTGGTCCGTCTGCAGCGCCAGCTGTGGGCGCGGCTGGCAGAAACGGAGC
CGGAGCTGCACCAACCCGGCGCCTCTCAACGGGGGCGCTTTCTGTGAGGGGCAGAATGTCCATGACCG
CACCGTCTCCTCTCTGCTTGTCTCTGTGGACGGCAGCTGGAGCCCGTGGAGCAAGTGGTCGGCCTGTG
GGCTGGACTGCACCCACTGGCGGAGCCGTGAGTGCTCTGACCCAGCACCCCGCAACGGAGGGGAGGAG
TGCCAGGGCACTGACCTGGACACCCGCAACTGTACCAGTGACCTCTGTGTACACAGTGCTTCTGGCCC
TGAGGACGTGGCCCTCTATGTGGGCCTCATCGCCGTGGCCGTCTGCCTGGTCCTGCTGCTGCTTGTCC
CCATCCTCGTTTATTGCCGGAAGAAGGAGGGGCTGGACTCAGATGTGGCTGACTCGTCCATTCTCACC
TCAGGCTTCCAGCCCGTCAGCATCAAGCCCAGCAAAGCAGACAACCCCCATCTGCTCACCATCCAGCC
GGACCTCAGCACCACCACCACCTACCAGGGCAGTCTCTGTCCCCGGCAGGATGGGCCCAGCCCCAAGT
TCCAGCTCACCAATGGGCACCTGCTCAGCCCCCTGGGTGGCGGCCGCCACACACTGCACCACAGCTCT
CCCACCTCTGAGGCCGAGGAGTTCGTCTCCCGCCTCTCCACCCAGAACTACTTCCGCTCCCTGCCCCG
GTATCAGCCTCCTCATCCCCCCAGATGCCATACCCCGAGGGAAGATCTATGAGATCTACCTCACGCTG
CACAAGCCGGAAGACGTGAGGTTGCCCCTAGCTGGCTGTCAGACCCTGCTGAGTCCCATCGTTAGCTG
TGGACCCCCTGGCGTCCTGCTCACCCGGCCAGTCATCCTGGCTATGGACCACTGTGGGGAGCCCAGCC
CTGACAGCTGGAGCCTGCGCCTCAAAAAGCAGTCGTGCGAGGGCAGCTGGGAGCAGGATGTGCTGCAC
CTGGGCGAGGAGGCGCCCTCCCACCTCTACTACTGCCAGCTGGAGGCCAGTGCCTGCTACGTCTTCAC
CGAGCAGCTGGGCCGCTTTGCCCTGGTGGGAGAGGCCCTCAGCGTGGCTGCCGCCAAGCGCCTCAAGC
TGCTTCTGTTTGCGCCGGTGGCCTGCACCTCCCTCGAGTACAACATCCGGGTCTACTGCCTGCATGAC
ACCCACGATGCACTCAAGGAGGTGGTGCAGCTGGAGAAGCAGCTGGGGGGACAGCTGATCCAGGAGCC
ACGGGTCCTGCACTTCAAGGACAGTTACCACAACCTGCGCCTATCCATCCACGATGTGCCCAGCTCCC
TGTGGAAGAGTAAGCTCCTTGTCAGCTACCAGGAGATCCCCTTTTATCACATCTGGAATGGCACGCAG
CGGTACTTGCACTGCACCTTCACCCTGGAGCGTGTCAGCCCCAGCACTAGTGACCTGGCCTGCAAGCT
GTGGGTGTGGCAGGTGGAGGGCGACGGGCAGAGCTTCAGCATCAACTTCAACATCACCAAGGACACAA
GGTTTGCTGAGCTGCTGGCTCTGGAGAGTGAAGCGGGGGTCCCAGCCCTGGTGGGCCCCAGTGCCTTC
AAGATCCCCTTCCTCATTCGGCAGAAGATAATTTCCAGCCTGGACCCACCCTGTAGGCGGGGTGCCGA
CCACAGCCATGATCCTCAACCTGTGGGAGGCGCGGCACTTCCCCAACGGCAACCTCAGCCAGCTGGCT
GCAGCAGTGGCTGGACTGGGCCAGCCAGACGCTGGCCTCTTCACAGTGTCGGAGGCTGAGTGCTGAGG
GGGGCCCTTCCCCACACCGGGGAGA

| | | |
|---|---|---|
| NOV21k, SNP13375492 of | SEQ ID NO: 526 | MW at 99046.9kD |

| | | | |
|---|---|---|---|
| CG53908-1, Protein Sequence | SNP Pos: 380 | 899 aa | SNP Change: Leu to Pro |

MAVRPGLWPALLGIVLAAWLRGSGAQQSATVANPVPGANPDLLPHFLVEPEDVYIVKNKPVLLVCKAV
PATQIFFKCNGEWVRQVDHVIERSTDGSSGEPTMEVRINVSRQQVEKVFGLEEYWCQCVAWSSSGTTK
SQKAYIRIARLRKNFEQEPLAKEVSLEQGIVLPCRPPEGIPPAEVEWLRNEDLVDPSLDPNVYITREH
SLVVRQARLADTANYTCVAKNIVARRRSASAAVIVYVNGGWSTWTEWSVCSASCGRGWQKRSRSCTNP
APLNGGAFCEGQNVHDRTVSSLLVSVDGSWSPWSKWSACGLDCTHWRSRECSDPAPRNGGEECQGTDL
DTRNCTSDLCVHSASGPEDVALYVGLIAVAVCLVLLLLVPILVYCRKKEGLDSDVADSSILTSGFQPV
SIKPSKADNPHLLTIQPDLSTTTTYQGSLCPRQDGPSPKFQLTNGHLLSPLGGGRHTLHHSSPTSEAE
EFVSRLSTQNYFRSLPRGTSNMTYGTFNFLGGRLMIPNTGISLLIPPDAIPRGKIYEIYLTLHKPEDV
RLPLAGCQTLLSPIVSCGPPGVLLTRPVILAMDHCGEPSPDSWSLRLKKQSCEGSWEQDVLHLGEEAP
SHLYYCQLEASACYVFTEQLGRFALVGEALSVAAAKRLKLLLFAPVACTSLEYNTRVYCLHDTHDALK
EVVQLEKQLGGQLIQEPRVLHFKDSYHNLRLSIHDVPSSLWKSKLLVSYQEIPFYHIWNGTQRYLHCT
FTLERVSPSTSDLACKLWVWQVEGDGQSFSINFNITKDTRFAELLALESEAGVPALVGPSAFKIPFLI
RQKIISSLDPPCRRGADWRTLLAQKLHLDSHLSFFASKPSPTAMILNLWEARHFPNGNLSQLAAAVAGL
GQPDAGLFTVSEAEC

| | |
|---|---|
| SEQ ID NO: 527 | 2881 bp |

| | | |
|---|---|---|
| NOV21l, SNP13375493 of | ORF Start: ATG at 87 | ORF Stop: TGA at 2784 |

| | | |
|---|---|---|
| CG53908-01, DNA Sequence | SNP Pos: 1660 | SNP Change: C to T |

AGCTGGGGCTCCGGGCTGAGGCGCTAAAGCCGCCCTCCCGCCCGCGGGGCCCCGCGCCCGGCCCGCCC
GCCTGCCCGCCCGCGGCCATGGCCGTCCGGCCCGGCCTGTGGCCAGCGCTCCTGGGCATAGTCCTCGC
CGCTTGGCTCCGCGGCTCGGGTGCCCAGCAGAGTGCCACCGTGGCCAACCCAGTGCCTGGTGCCAACC
CGGACCTGCTTCCCCACTTCCTGGTGGAGCCCGAGGATGTGTACATCGTCAAGAACAAGCCAGTGCTG
CTTGTGTGCAAGGCCGTGCCCGCCACGCAGATCTTCTTCAAGTGCAACGGGGAGTGGGTGCGCCAGGT
GGACCACGTGATCGAGCGCAGCACAGACGGGAGCAGTGGTGAGCCGACCATGGAGGTCCGCATTAATG
TCTCAAGGCAGCAGGTCGAGAAGGTGTTCGGGCTGGAGGAATACTGGTGCCAGTGCGTGGCATGGAGC
TCCTCGGGCACCACCAAGAGTCAGAAGGCCTACATCCGCATAGCCAGATTGCGCAAGAACTTCGAGCA
GGAGCCGCTGGCCAAGGAGGTGTCCCTGGAGCAGGGCATCGTGCTGCCCTGCCGTCCACCGGAGGGCA
TCCCTCCAGCCGAGGTGGAGTGGCTCCGGAACGAGGACCTGGTGGACCCGTCCCTGGACCCCAATGTA
TACATCACGCGGGAGCACAGCCTGGTGGTGCGACAGGCCCGCCTTGCTGACACGGCCAACTACACCTG
CGTGGCCAAGAACATCGTGGCACGTCGCCGCAGCGCCTCCGCTGCTGTCATCGTCTACGTGAACGGTG
GGTGGTCGACGTGGACCGAGTGGTCCGTCTGCAGCGCCAGCTGTGGGCGCGGCTGGCAGAAACGGAGC
CGGAGCTGCACCAACCCGGCGCCTCTCAACGGGGGCGCTTTCTGTGAGGGGCAGAATGTCCATGACCG
CACCGTCTCCTCTCTGCTTGTCTCTGTGGACGGCAGCTGGAGCCCGTGGAGCAAGTGGTCGGCCTGTG
GGCTGGACTGCACCCACTGGCGGAGCCGTGAGTGCTCTGACCCAGCACCCCGCAACGGAGGGGAGGAG
TGCCAGGGCACTGACCTGGACACCCGCAACTGTACCAGTGACCTCTGTGTACACAGTGCTTCTGGCCC
TGAGGACGTGGCCCTCTATGTGGGCCTCATCGCCGTGGCCGTCTGCCTGGTCCTGCTGCTGCTTGTCC
TCATCCTCGTTTATTGCCGGAAGAAGGAGGGGCTGGACTCAGATGTGGCTGACTCGTCCATTCTCACC
GGACCTCAGCACCACCACCACCTACCAGGGCAGTCTCTGTCCCCGGCAGGATGGGCCCAGCCCCAAGT
TCCAGCTCACCAATGGGCACCTGCTCAGCCCCCTGGGTGGCGGCCGCCACACACTGCACCACAGCTCT
GTATCAGCCTCCTCATCCCCCCAGATGTCATACCCCGAGGGAAGATCTATGAGATCTACCTCACGCTG
CACAAGCCGGAAGACGTGAGGTTGCCCCTAGCTGGCTGTCAGACCCTGCTGAGTCCCATCGTTAGCTG
TGGACCCCCTGGCGTCCTGCTCACCCGGCCAGTCATCCTGGCTATGGACCACTGTGGGGAGCCCAGCC
CTGACAGCTGGAGCCTGCGCCTCAAAAAGCAGTCGTGCGAGGGCAGCTGGGAGCAGGATGTGCTGCAC
CTGGGCGAGGAGGCGCCCTCCCACCTCTACTACTGCCAGCTGGAGGCCAGTGCCTGCTACGTCTTCAC
CGAGCAGCTGGGCCGCTTTGCCCTGGTGGGAGAGGCCCTCAGCGTGGCTGCCGCCAAGCGCCTCAAGC
TGCTTCTGTTTGCGCCGGTGGCCTGCACCTCCCTCGAGTACAACATCCGGGTCTACTGCCTGCATGAC
ACCCACGATGCACTCAAGGAGGTGGTGCAGCTGGAGAAGCAGCTGGGGGGACAGCTGATCCAGGAGCC
ACGGGTCCTGCACTTCAAGGACAGTTACCACAACCTGCGCCTATCCATCCACGATGTGCCCAGCTCCC
TGTGGAAGAGTAAGCTCCTTGTCAGCTACCAGGAGATCCCCTTTTATCACATCTGGAATGGCACGCAG
CGGTACTTGCACTGCACCTTCACCCTGGAGCGTGTCAGCCCCAGCACTAGTGACCTGGCCTGCAAGCT
GTGGGTGTGGCAGGTGGAGGGCGACGGGCAGAGCTTCAGCATCAACTTCAACATCACCAAGGACACAA
GGTTTGCTGAGCTGCTGGCTCTGGAGAGTGAAGCGGGGGTCCCAGCCCTGGTGGGCCCCAGTGCCTTC
AAGATCCCCTTCCTCATTCGGCAGAAGATAATTTCCAGCCTGGACCCACCCTGTAGGCGGGGTGCCGA
CCACAGCCATGATCCTCAACCTGTGGGAGGCGCGGCACTTCCCCAACGGCAACCTCAGCCAGCTGGCT
GCAGCAGTGGCTGGACTGGGCCAGCCAGACGCTGGCCTCTTCACAGTGTCGGAGGCTGAGTGCTGAGG
CCGGCCAGGCCCGACACCTACACTCTCACCAGCTTTGGCACCCACCAAGGACAGGCAGAAGCCGGACA
GGGGCCCTTCCCCACACCGGGGAGA

| | | |
|---|---|---|
| NOV21l, SNP13375493 of | SEQ ID NO: 528 | MW at 99091.0kD |

| | | | |
|---|---|---|---|
| CG53908-01, Protein Sequence | SNP Pos: 525 | 899 aa | SNP Change: Ala to Val |

MARRPGLWPALLGIVLAAWLRGSGAQQSATVANPVPGANPDLLPHFLVEPEDVYIVKNKPVLLVCKAV
PATQIFFKCNGEWVRQVDHVIERSTDGSSGEPTMEVRINVSRQQVEKVFGLEEYWCQCVAWSSSGTTK
SQKAYIRIARLRKNFEQEPLAKEVSLEQGIVLPCRPPEGIPPAEVEWLRNEDLVDPSLDPNVYITREH
SLVVRQARLADTANYTCVAKNIVARRRSASAAVIVYVNGGWSTWTEWSVCSASCGRGWQKRSRSCTNP
APLNGGAFCEGQNVHDRTVSSLLVSVDGSWSPWSKWSACGLDCTHWRSRECSDPAPRNGGEECQGTDL
DTRNCTSDLCVHSASGPEDVALYVGLIAVAVCLVLLLLVILVYCRKKEGLDSDVADSSILTSGFQPV
SIKPSKADNPHLLTIQPDLSTTTTYQGSLCPRQDGPSPKFQLTNGHLLSPLGGGRHTLHHSSPTSEAE
EFVSRLSTQNYFRSLPRGTSNMTYGTFNFLGGRLMIPNTGISLLIPPDVIPRGKIYEIYLTLHKPEDV
RLPLAGCQTLLSPIVSCGPPGVLLTRPVILAMDHCGEPSPDSWSLRLKKQSCEGSWEQDVLHLGEEAP
SHLYYCQLEASACYVFTEQLGRFALVGEALSVAAAKRLKLLLFAPVACTSLEYNIRVYCLHDTHDALK
EVVQLEKQLGGQLIQEPRVLHFKDSYHNLRLSIHDVPSSLWKSKLLVSYQEIPFYHIWNGTQRYLHCT
FTLERVSPSTSDLACKLWVWQVEGDGQSFSINFNITKDTRFAELLALESEAGVPALVGPSAFKTPFLI
RQKIISSLDPPCRRGADWRTLAQKLHLDSHLSFFASKPSPTAMILNLWEARHFPNGNLSQLAAAVAGL
GQPDAGLFTVSEAEC

| | |
|---|---|
| SEQ ID NO: 529 | 2881 bp |

| | | |
|---|---|---|
| NOV21m, SNP13375135 of | ORF Start: ATG at 87 | ORF Stop: TGA at 2784 |

| | | |
|---|---|---|
| CG53908-01, DNA Sequence | SNP Pos: 1855 | SNP Change: G to A |

AGCTGGGGCTCCGGGCTGAGGCGCTAAAGCCGCCCTCCCGCCCGCGGGGCCCCGCGCCCGGCCCGCCC
GCCTGCCCGCCCGCGGCCATGGCCGTCCGGCCCGGCCTGTGGCCAGCGCTCCTGGGCATAGTCCTCGC
CGCTTGGCTCCGCGGCTCGGGTGCCCAGCAGAGTGCCACCGTGGCCAACCCAGTGCCTGGTGCCAACC
CGGACCTGCTTCCCCACTTCCTGGTGGAGCCCGAGGATGTGTACATCGTCAAGAACAAGCCAGTGCTG
CTTGTGTGCAAGGCCGTGCCCGCCACGCAGATCTTCTTCAAGTGCAACGGGGAGTGGGTGCGCCAGGT
GGACCACGTGATCGAGCGCAGCACAGACGGGAGCAGTGGTGAGCCGACCATGGAGGTCCGCATTAATG
TCTCAAGGCAGCAGGTCGAGAAGGTGTTCGGGCTGGAGGAATACTGGTGCCAGTGCGTGGCATGGAGC
TCCTCGGGCACCACCAAGAGTCAGAAGGCCTACATCCGCATAGCCAGATTGCGCAAGAACTTCGAGCA
GGAGCCGCTGGCCAAGGAGGTGTCCCTGGAGCAGGGCATCGTGCTGCCCTGCCGTCCACCGGAGGGCA
TACATCACGCGGGAGCACAGCCTGGTGGTGCGACAGGCCCGCCTTGCTGACACGGCCAACTACACCTG
CGTGGCCAAGAACATCGTGGCACGTCGCCGCAGCGCCTCCGCTGCTGTCATCGTCTACGTGAACGGTG
GGTGGTCGACGTGGACCGAGTGGTCCGTCTGCAGCGCCAGCTGTGGGCGCGGCTGGCAGAAACGGAGC
CACCGTCTCCTCTCTGCTTGTCTCTGTGGACGGCAGCTGGAGCCCGTGGAGCAAGTGGTCGGCCTGTG
TGCCAGGGCACTGACCTGGACACCCGCAACTGTACCAGTGACCTCTGTGTACACAGTGCTTCTGGCCC
TGAGGACGTGGCCCTCTATGTGGGCCTCATCGCCGTGGCCGTCTGCCTGGTCCTGCTGCTGCTTGTCC
TCATCCTCGTTTATTGCCGGAAGAAGGAGGGGCTGGACTCAGATGTGGCTGACTCGTCCATTCTCACC
TCAGGCTTCCAGCCCGTCAGCATCAAGCCCAGCAAAGCAGACAACCCCCATCTGCTCACCATCCAGCC
GGACCTCAGCACCACCACCACCTACCAGGGCAGTCTCTGTCCCCGGCAGGATGGGCCCAGCCCCAAGT
TCCAGCTCACCAATGGGCACCTGCTCAGCCCCCTGGGTGGCGGCCGCCACACACTGCACCACAGCTCT
CCCACCTCTGAGGCCGAGGAGTTCGTCTCCCGCCTCTCCACCCAGAACTACTTCCGCTCCCTGCCCCG
AGGCACCAGCAACATGACCTATGGGACCTTCAACTTCCTCGGGGGCCGGCTGATGATCCCTAATACAG
GTATCAGCCTCCTCATCCCCCCAGATGCCATACCCCGAGGGAAGATCTATGAGATCTACCTCACGCTG
CACAAGCCGGAAGACGTGAGGTTGCCCCTAGCTGGCTGTCAGACCCTGCTGAGTCCCATCGTTAGCTG
TGGACCCCCTGGCGTCCTGCTCACCCGGCCAGTCATCCTGGCTATGGACCACTGTGGGGAGCCCAGCC
CTGACAGCTGGAGCCTGCACCTCAAAAAGCAGTCGTGCGAGGGCAGCTGGGAGCAGGATGTGCTGCAC
CTGGGCGAGGAGGCGCCCTCCCACCTCTACTACTGCCAGCTGGAGGCCAGTGCCTGCTACGTCTTCAC
CGAGCAGCTGGGCCGCTTTGCCCTGGTGGGAGAGGCCCTCAGCGTGGCTGCCGCCAAGCGCCTCAAGC
TGCTTCTGTTTGCGCCGGTGGCCTGCACCTCCCTCGAGTACAACATCCGGGTCTACTGCCTGCATGAC
ACCCACGATGCACTCAAGGAGGTGGTGCAGCTGGAGAAGCAGCTGGGGGGACAGCTGATCCAGGAGCC
ACGGGTCCTGCACTTCAAGGACAGTTACCACAACCTGCGCCTATCCATCCACGATGTGCCCAGCTCCC
TGTGGAAGAGTAAGCTCCTTGTCAGCTACCAGGAGATCCCCTTTTATCACATCTGGAATGGCACGCAG
CGGTACTTGCACTGCACCTTCACCCTGGAGCGTGTCAGCCCCAGCACTAGTGACCTGGCCTGCAAGCT
GTGGGTGTGGCAGGTGGAGGGCGACGGGCAGAGCTTCAGCATCAACTTCAACATCACCAAGGACACAA
GGTTTGCTGAGCTGCTGGCTCTGGAGAGTGAAGCGGGGGTCCCAGCCCTGGTGGGCCCCAGTGCCTTC
AAGATCCCCTTCCTCATTCGGCAGAAGATAATTTCCAGCCTGGACCCACCCTGTAGGCGGGGTGCCGA
CTGGCGGACTCTGGCCCAGAAACTCCACCTGGACAGCCATCTCAGCTTCTTTGCCTCCAAGCCCAGCC
CCACAGCCATGATCCTCAACCTGTGGGAGGCGCGGCACTTCCCCAACGGCAACCTCAGCCAGCTGGCT
GCAGCAGTGGCTGGACTGGGCCAGCCAGACGCTGGCCTCTTCACAGTGTCGGAGGCTGAGTGCTGAGG
CCGGCCAGGCCCGACACCTACACTCTCACCAGCTTTGGCACCCACCAAGGACAGGCAGAAGCCGGACT
GGGGCCCTTCCCCACACCGGGGAGA

| | | |
|---|---|---|
| NOV21m, SNP13375135 of | SEQ ID NO:530 | MW at 99043.9kD |

| | | | |
|---|---|---|---|
| CG53908-01, Protein Sequence | SNP Pos: 590 | 899 aa | SNP Change: Arg to His |

MAVRPGLWPALLGIVLAAWLRGSGAQQSATVANPVPGANPDLLPHFLVEPEDVYIVKNKPVLLVCKAV
PATQIFFKCNGEWVRQVDHVIERSTDGSSGEPTMEVRINVSRQQVEKVFGLEEYWCQCVAWSSSGTTK
SQKAYIRIARLRKNFEQEPLAKEVSLEQGIVLPCRPPEGIPPAEVEWLRNEDLVDPSLDPNVYITREH
SLVVRQARLADTANYTCVAKNIVARRRSASAAVIVYVNGGWSTWTEWSVCSASCGRGWQKRSRSCTNP
APLNGGAFCEGQNVHDRTVSSLLVSVDGSWSPWSKWSACGLDCTHWRSRECSDPAPRNGGEECQGTDL
DTRNCTSDLCHSASGPEDVALYVGLIAVAVCLVLLLLVLILVYCRKKEGLDSDVPINSSILTSGFQPV
SIKPSKADNPHLLTIQPDLSTTTTYQGSLCPRQDGPSPKFQLTNGHLLSPLGGGRHTLHHSSPTSEAE
EFVSRLSTQNYFRSLPRGTSNMTYGTFNFLGGRLMIPNTGISLLIPPDAIPRGKIYEIYLTLHKPEDV
RLPLAGCQTLLSPIVSCGPPGVLLTRPVILAMDHCGEPSPDSWSLHLKKQSCEGSWEQDVLHLGEEAP
SHLYYCQLEASACYVFTEQLGRFALVGEALSVAAAKRLKLLLFAPVACTSLEYNIRVYCLHDTHDALK
EVVQLEKQLGGQLIQEPRVLHFKDSYHNLRLSIHDVPSSLWKSKLLVSYQEIPFYHIWNGTQRYLHCT
FTLERVSPSTSDLACKLWVWQVEGDGQSFSINFNITKDTRFAELLALESEAGVPALVGPSAFKIPFLI
RQKIISSLDPPCRRGADWRTLAQKLHLDSHLSFFASKPSPTAMILNLWEARHFPNGNLSQLAAAVAGL
GQPDAGLFTVSEAEC

| | |
|---|---|
| SEQ ID NO: 531 | 2881 bp |

| | | |
|---|---|---|
| NOV21n, SNP13375136 of | ORF Start: ATG at 87 | ORF Stop: TGA at 2784 |

| | | |
|---|---|---|
| CG53908-01, DNA Sequence | SNP Pos: 1965 | SNP Change: G to A |

AGCTGGGGCTCCGGGCTGAGGCGCTAAAGCCGCCCTCCCGCCCGCGGGGCCCCGCGCCCGGCCCGCCC
CGCTTGGCTCCGCGGCTCGGGTGCCCAGCAGAGTGCCACCGTGGCCAACCCAGTGCCTGGTGCCAACC
CGGACCTGCTTCCCCACTTCCTGGTGGAGCCCGAGGATGTGTACATCGTCAAGAACAAGCCAGTGCTG
CTTGTGTGCAAGGCCGTGCCCGCCACGCAGATCTTCTTCAAGTGCAACGGGGAGTGGGTGCGCCAGGT
GGACCACGTGATCGAGCGCAGCACAGACGGGAGCAGTGGTGAGCCGACCATGGAGGTCCGCATTAATG
TCTCAAGGCAGCAGGTCGAGAAGGTGTTCGGGCTGGAGGAATACTGGTGCCAGTGCGTGGCATGGAGC
TCCTCGGGCACCACCAAGAGTCAGAAGGCCTACATCCGCATAGCCAGATTGCGCAAGAACTTCGAGCA
GGAGCCGCTGGCCAAGGAGGTGTCCCTGGAGCAGGGCATCGTGCTGCCCTGCCGTCCACCGGAGGGCA
TCCCTCCAGCCGAGGTGGAGTGGCTCCGGAACGAGGACCTGGTGGACCCGTCCCTGGACCCCAATGTA
TACATCACGCGGGAGCACAGCCTGGTGGTGCGACAGGCCCGCCTTGCTGACACGGCCAACTACACCTG
CGTGGCCAAGAACATCGTGGCACGTCGCCGCAGCGCCTCCGCTGCTGTCATCGTCTACGTGAACGGTG
GGTGGTCGACGTGGACCGAGTGGTCCGTCTGCAGCGCCAGCTGTGGGCGCGGCTGGCAGAAACGGAGC
CGGAGCTGCACCAACCCGGCGCCTCTCAACGGGGGCGCTTTCTGTGAGGGGCAGAATGTCCATGACCG
CACCGTCTCCTCTCTGCTTGTCTCTGTGGACGGCAGCTGGAGCCCGTGGAGCAAGTGGTCGGCCTGTG
GGCTGGACTGCACCCACTGGCGGAGCCGTGAGTGCTCTGACCCAGCACCCCGCAACGGAGGGGAGGAG
TGCCAGGGCACTGACCTGGACACCCGCAACTGTACCAGTGACCTCTGTGTACACAGTGCTTCTGGCCC
TGAGGACGTGGCCCTCTATGTGGGCCTCATCGCCGTGGCCGTCTGCCTGGTCCTGCTGCTGCTTGTCC
TCATCCTCGTTTATTGCCGGAAGAAGGAGGGGCTGGACTCAGATGTGGCTGACTCGTCCATTCTCACC
TCAGGCTTCCAGCCCGTCAGCATCAAGCCCAGCAAAGCAGACAACCCCCATCTGCTCACCATCCAGCC
GGACCTCAGCACCACCACCACCTACCAGGGCAGTCTCTGTCCCCGGCAGGATGGGCCCAGCCCCAAGT
TCCAGCTCACCAATGGGCACCTGCTCAGCCCCCTGGGTGGCGGCCGCCACACACTGCACCACAGCTCT
CCCACCTCTGAGGCCGAGGAGTTCGTCTCCCGCCTCTCCACCCAGAACTACTTCCGCTCCCTGCCCCG
AGGCACCAGCAACATGACCTATGGGACCTTCAACTTCCTCGGGGGCCGGCTGATGATCCCTAATACAG
CACAAGCCGGAAGACGTGAGGTTGCCCCTAGCTGGCTGTCAGACCCTGCTGAGTCCCATCGTTAGCTG
TGGACCCCCTGGCGTCCTGCTCACCCGGCCAGTCATCCTGGCTATGGACCACTGTGGGGAGCCCAGCC
CTGACAGCTGGAGCCTGCGCCTCAAAAAGCAGTCGTGCGAGGGCAGCTGGGAGCAGGATGTGCTGCAC
CTGGGCGAGGAGGCGCCCTCCCACCTCTACTACTGCCAGCTGGAGGCCAGTGCCTGCTACATCTTCAC
CGAGCAGCTGGGCCGCTTTGCCCTGGTGGGAGAGGCCCTCAGCGTGGCTGCCGCCAAGCGCCTCAAGC
ACCCACGATGCACTCAAGGAGGTGGTGCAGCTGGAGAAGCAGCTGGGGGGACAGCTGATCCAGGAGCC
TGTGGAAGAGTAAGCTCCTTGTCAGCTACCAGGAGATCCCCTTTTATCACATCTGGAATGGCACGCAG
CGGTACTTGCACTGCACCTTCACCCTGGAGCGTGTCAGCCCCAGCACTAGTGACCTGGCCTGCAAGCT
GTGGGTGTGGCAGGTGGAGGGCGACGGGCAGAGCTTCAGCATCAACTTCAACATCACCAAGGACACAA
GGTTTGCTGAGCTGCTGGCTCTGGAGAGTGAAGCGGGGGTCCCAGCCCTGGTGGGCCCCAGTGCCTTC
AAGATCCCCTTCCTCATTCGGCAGAAGATAATTTCCAGCCTGGACCCACCCTGTAGGCGGGGTGCCGA
CTGGCGGACTCTGGCCCAGAAACTCCACCTGGACAGCCATCTCAGCTTCTTTGCCTCCAAGCCCAGCC
CCACAGCCATGATCCTCAACCTGTGGGAGGCGCGGCACTTCCCCAACGGCAACCTCAGCCAGCTGGCT
GCAGCAGTGGCTGGACTGGGCCAGCCAGACGCTGGCCTCTTCACAGTGTCGGAGGCTGAGTGCTGAGG
CCGGCCAGGCCCGACACCTACACTCTCACCAGCTTTGGCACCCACCAAGGACAGGCAGAAGCCGGACA
GGGGCCCTTCCCCACACCGGGGAGA

| | | |
|---|---|---|
| NOV21n, SNP13375136 of | SEQ ID NO: 532 | MW at 99077.0kD |

| | | | |
|---|---|---|---|
| CG53908-01, Protein Sequence | SNP Pos: 627 | 899 aa | SNP Change: Val to Ile |

+TL,1, MARVPGLWPALLGIVLAAWLRGSGAQQSATVANPVPGANPDLLPHFLVEPEDVYIVKNKPVLLVCKAV
PATQIFFKCNGEWVRQVDHVIERSTDGSSGEPTMEVRINVSRQQVEKVFGLEEYWCQCVAWSSSGTTK
SQKAYIRIARLRKNFEQEPLAKEVSLEQGIVLPCRPPEGIPPAEVEWLRNEDLVDPSLDPNVYITREH
DTRNCTSDLCVHSASGPEDVALYVGLIAVAVCLVLLLLVLILVYCRKKEGLDSDVADSSILTSGFQPV
SIKPSKADNPHLLTIQPDLSTTTTYQGSLCPRQDGPSPKFQLTNGHLLSPLGGGRHTLHHSSPTSEAE
EFVSRLSTQNYFRSLPRGTSNMTYGTFNFLGGRLMIPNTGISLLIPPDAIPRGKIYEIYLTLHKPEDV
RLPLAGCQTLLSPIVSCGPPGVLLTRPVILAMDHCGEPSPDSWSLRLKYQSCEGSWEQDVLHLGEEAP
SHLYYCQLEASACYIFTEQLGRFALVGEALSVAAAKRLKLLLFAPVACTSLEYNIRVYCLHDTHDALK
EVVQLEKQLGGQLIQEPRVLHFKDSYHNLRLSIHDVPSSLWKSKLLVSYQEIPFYHIWNGTQRYLHCT
FTLERVSPSTSDLACKLWVWQVEGDGQSFSINFNITKDTRFAELLALESEAGVPALVGPSAFKIPFLI
RQKIISSLDPPCRRGADWRTLAQKLHLDSHLSFFASKPSPTAMILNLWEARHFPNGNLSQLAAAVAGL
GQPDAGLFTVSEAEC

| | |
|---|---|
| SEQ ID NO: 533 | 2881 bp |

| | | |
|---|---|---|
| NOV21o, SNP13375137 of | ORF Start: ATG at 87 | ORF Stop: TGA at 2784 |

| | | |
|---|---|---|
| CG53908-01, DNA Sequence | SNP Pos: 2025 | SNP Change: G to A |

AGCTGGGGCTCCGGGCTGAGGCGCTAAAGCCGCCCTCCCGCCCGCGGGGCCCCGCGCCCGGCCCGCCC
GCCTGCCCGCCCGCGGCCATGGCCGTCCGGCCCGGCCTGTGGCCAGCGCTCCTGGGCATAGTCCTCGC
CGCTTGGCTCCGCGGCTCGGGTGCCCAGCAGAGTGCCACCGTGGCCAACCCAGTGCCTGGTGCCAACC
CGGACCTGCTTCCCCACTTCCTGGTGGAGCCCGAGGATGTGTACATCGTCAAGAACAAGCCAGTGCTG
CTTGTGTGCAAGGCCGTGCCCGCCACGCAGATCTTCTTCAAGTGCAACGGGGAGTGGGTGCGCCAGGT
GGACCACGTGATCGAGCGCAGCACAGACGGGAGCAGTGGTGAGCCGACCATGGAGGTCCGCATTAATG
TCTCAAGGCAGCAGGTCGAGAAGGTGTTCGGGCTGGAGGAATACTGGTGCCAGTGCGTGGCATGGAGC
TCCTCGGGCACCACCAAGAGTCAGAAGGCCTACATCCGCATAGCCAGATTGCGCAAGAACTTCGAGCA
GGAGCCGCTGGCCAAGGAGGTGTCCCTGGAGCAGGGCATCGTGCTGCCCTGCCGTCCACCGGAGGGCA
TCCCTCCAGCCGAGGTGGAGTGGCTCCGGAACGAGGACCTGGTGGACCCGTCCCTGGACCCCAATGTA
TACATCACGCGGGAGCACAGCCTGGTGGTGCGACAGGCCCGCCTTGCTGACACGGCCAACTACACCTG
CGTGGCCAAGAACATCGTGGCACGTCGCCGCAGCGCCTCCGCTGCTGTCATCGTCTACGTGAACGGTG
GGTGGTCGACGTGGACCGAGTGGTCCGTCTGCAGCGCCAGCTGTGGGCGCGGCTGGCAGAAACGGAGC
CGGAGCTGCACCAACCCGGCGCCTCTCAACGGGGGCGCTTTCTGTGAGGGGCAGAATGTCCATGACCG
CACCGTCTCCTCTCTGCTTGTCTCTGTGGACGGCAGCTGGAGCCCGTGGAGCAAGTGGTCGGCCTGTG
GGCTGGACTGCACCCACTGGCGGAGCCGTGAGTGCTCTGACCCAGCACCCCGCAACGGAGGGGAGGAG
TGCCAGGGCACTGACCTGGACACCCGCAACTGTACCAGTGACCTCTGTGTACACAGTGCTTCTGGCCC
TGAGGACGTGGCCCTCTATGTGGGCCTCATCGCCGTGGCCGTCTGCCTGGTCCTGCTGCTGCTTGTCC
TCATCCTCGTTTATTGCCGGAAGAAGGAGGGGCTGGACTCAGATGTGGCTGACTCGTCCATTCTCACC
TCAGGCTTCCAGCCCGTCAGCATCAAGCCCAGCAAAGCAGACAACCCCCATCTGCTCACCATCCAGCC
GGACCTCAGCACCACCACCACCTACCAGGGCAGTCTCTGTCCCCGGCAGGATGGGCCCAGCCCCAAGT
TCCAGCTCACCAATGGGCACCTGCTCAGCCCCCTGGGTGGCGGCCGCCACACACTGCACCACAGCTCT
CCCACCTCTGAGGCCGAGGAGTTCGTCTCCCGCCTCTCCACCCAGAACTACTTCCGCTCCCTGCCCCG
AGGCACCAGCAACATGACCTATGGGACCTTCAACTTCCTCGGGGGCCGGCTGATGATCCCTAATACAG
GTATCAGCCTCCTCATCCCCCCAGATGCCATACCCCGAGGGAAGATCTATGAGATCTACCTCACGCTG
TGGACCCCCTGGCGTCCTGCTCACCCGGCCAGTCATCCTGGCTATGGACCACTGTGGGGAGCCCAGCC
CTGACAGCTGGAGCCTGCGCCTCAAAAAGCAGTCGTGCGAGGGCAGCTGGGAGCAGGATGTGCTGCAC
CTGGGCGAGGAGGCGCCCTCCCACCTCTACTACTGCCAGCTGGAGGCCAGTGCCTGCTACGTCTTCAC
CGAGCAGCTGGGCCGCTTTGCCCTGGTGGGAGAGGCCCTCAGCGTGGCTGCCACCAAGCGCCTCAAGC
TGCTTCTGTTTGCGCCGGTGGCCTGCACCTCCCTCGAGTACAACATCCGGGTCTACTGCCTGCATGAC
ACCCACGATGCACTCAAGGAGGTGGTGCAGCTGGAGAAGCAGCTGGGGGGACAGCTGATCCAGGAGCC
ACGGGTCCTGCACTTCAAGGACAGTTACCACAACCTGCGCCTATCCATCCACGATGTGCCCAGCTCCC
TGTGGAAGAGTAAGCTCCTTGTCAGCTACCAGGAGATCCCCTTTTATCACATCTGGAATGGCACGCAG
CGGTACTTGCACTGCACCTTCACCCTGGAGCGTGTCAGCCCCAGCACTAGTGACCTGGCCTGCAAGCT
GTGGGTGTGGCAGGTGGAGGGCGACGGGCAGAGCTTCAGCATCAACTTCAACATCACCAAGGACACAA
GGTTTGCTGAGCTGCTGGCTCTGGAGAGTGAAGCGGGGGTCCCAGCCCTGGTGGGCCCCAGTGCCTTC
AAGATCCCCTTCCTCATTCGGCAGAAGATAATTTCCAGCCTGGACCCACCCTGTAGGCGGGGTGCCGA
CTGGCGGACTCTGGCCCAGAAACTCCACCTGGACAGCCATCTCAGCTTCTTTGCCTCCAAGCCCAGCC
CCACAGCCATGATCCTCAACCTGTGGGAGGCGCGGCACTTCCCCAACGGCAACCTCAGCCAGCTGGCT
GCAGCAGTGGCTGGACTGGGCCAGCCAGACGCTGGCCTCTTCACAGTGTCGGAGGCTGAGTGCTGAGG
CCGGCCAGGCCCGACACCTACACTCTCACCAGCTTTGGCACCCACCAAGGACAGGCAGAAGCCGGACA
GGGGCCCTTCCCCACACCGCGGAGA

| | | |
|---|---|---|
| NOV21o, SNP13375137 of | SEQ ID NO: 534 | MW at 99093.0kD |

| | | | |
|---|---|---|---|
| CG53908-01, Protein Sequence | SNP Pos: 647 | 899 aa | SNP Change: Ala to Thr |

MAVRPGLWPALLGIVLAAWLRGSGAQQSATVANPVPGANPDLLPHFLVEPEDVYIVKNKPVLLVCKAV
PATQIFFKCNGEWVRQVDHVIERSTDGSSGEPTMEVRINVSRQQVEKVFGLEEYWCQCVAWSSSGTTK
SQKAYIRIARLRKNFEQEPLAKEVSLEQGIVLPCRPPEGIPPAEVEWLRNEDLVDPSLDPNVYITREH
SLVVRQARLADTANYTCVAKNIVARRRSASAAVIVYVNGGWSTWTEWSVCSASCGRGWQKRSRSCTNP
APLNGGAFCEGQNVHDRTVSSLLVSVDGSWSPWSKWSACGLDCTHWRSRECSDPAPRNGGEECQGTDL
DTRNCTSDLCVHSASGPEDVALYVGLIAVAVCLVLLLLVLILVYCRKKEGLDSDVADSSILTSGFQPV
SIKPSKADNPHLLTIQPDLSTTTTYQGSLCPRQDGPSPKFQLTNGHLLSPLGGGRHTLHHSSPTSEAE
EFVSRLSTQNYFRSLPRGTSNMTYGTFNFLGGPLMIPNTGISLLIPPDAIPRGKIYEIYLTLHKPEDV
RLPLAGCQTLLSPIVSCGPPGVLLTRPVILAMDHCGEPSPDSWSLRLKKQSCEGSWEQDVLHLGEEAP
SHLYYCQLEASACYVFTEQLGRFALVGEALSVAATKRLKLLLFAPVACTSLEYNIRVYCLHDTHDALK
EVVQLEKQLGGQLIQEPRVLHFKDSYHNLRLSIHDVPSSLWKSKLLVSYQEIPFYHIWNGTQRYLHCT
FTLERVSPSTSDLACKLWVWQVEGDGQSFSINFNITKDTRFAELLALESEAGVPALVGPSAFKIPFLI
RQKIISSLDPPCRRGADWRTLAQKLHLDSHLSFFASKPSPTAMILNLWEARHFPNGNLSQLAAAVAGL
GQPDAGLFTVSEAEC

| | |
|---|---|
| SEQ ID NO: 535 | 2881 bp |

| | | |
|---|---|---|
| NOV21p, SNP13375138 of | ORF Start: ATG at 87 | ORF Stop: TGA at 2784 |

| | | |
|---|---|---|
| CG53908-01, DNA Sequence | SNP Pos: 2038 | SNP Change: A to G |

AGCTGGGGCTCCGGGCTGAGGCGCTAAAGCCGCCCTCCCGCCCGCGGGGCCCCGCGCCCGGCCCGCCC
GCCTGCCCGCCCGCGGCCATGGCCGTCCGGCCCGGCCTGTGGCCAGCGCTCCTGGGCATAGTCCTCGC
CGCTTGGCTCCGCGGCTCGGGTGCCCAGCAGAGTGCCACCGTGGCCAACCCAGTGCCTGGTGCCAACC
CGGACCTGCTTCCCCACTTCCTGGTGGAGCCCGAGGATGTGTACATCGTCAAGAACAAGCCAGTGCTG
CTTGTGTGCAAGGCCGTGCCCGCCACGCAGATCTTCTTCAAGTGCAACGGGGAGTGGGTGCGCCAGGT
GGACCACGTGATCGAGCGCAGCACAGACGGGAGCAGTGGTGAGCCGACCATGGAGGTCCGCATTAATG
TCTCAAGGCAGCAGGTCGAGAAGGTGTTCGGGCTGGAGGAATACTGGTGCCAGTGCGTGGCATGGAGC
TCCTCGGGCACCACCAAGAGTCAGAAGGCCTACATCCGCATAGCCAGATTGCGCAAGAACTTCGAGCA
GGAGCCGCTGGCCAAGGAGGTGTCCCTGGAGCAGGGCATCGTGCTGCCCTGCCGTCCACCGGAGGGCA
TCCCTCCAGCCGAGGTGGAGTGGCTCCGGAACGAGGACCTGGTGGACCCGTCCCTGGACCCCAATGTA
CGTGGCCAAGAACATCGTGGCACGTCGCCGCAGCGCCTCCGCTGCTGTCATCGTCTACGTGAACGGTG
GGTGGTCGACGTGGACCGAGTGGTCCGTCTGCAGCGCCAGCTGTGGGCGCGGCTGGCAGAAACGGAGC
CGGAGCTGCACCAACCCGGCGCCTCTCAACGGGGGCGCTTTCTGTGAGGGGCAGAATGTCCATGACCG
CACCGTCTCCTCTCTGCTTGTCTCTGTGGACGGCAGCTGGAGCCCGTGGAGCCCGTGGTCGGCCTGTG
GGCTGGACTGCACCCACTGGCGGAGCCGTGAGTGCTCTGACCCAGCACCCCGCAACGGAGGGGAGGAG
TGCCAGGGCACTGACCTGGACACCCGCAACTGTACCAGTGACCTCTGTGTACACAGTGCTTCTGGCCC
TGAGGACGTGGCCCTCTATGTGGGCCTCATCGCCGTGGCCGTCTGCCTGGTCCTGCTGCTGCTTGTCC
TCATCCTCGTTTATTGCCGGAAGAAGGAGGGGCTGGACTCAGATGTGGCTGACTCGTCCATTCTCACC
TCAGGCTTCCAGCCCGTCAGCATCAAGCCCAGCAAAGCAGACAACCCCCATCTGCTCACCATCCAGCC
GGACCTCAGCACCACCACCACCTACCAGGGCAGTCTCTGTCCCCGGCAGGATGGGCCCAGCCCCAAGT
TCCAGCTCACCAATGGGCACCTGCTCAGCCCCCTGGGTGGCGGCCGCCACACACTGCACCACAGCTCT
CCCACCTCTGAGGCCGAGGAGTTCGTCTCCCGCCTCTCCACCCAGAACTACTTCCGCTCCCTGCCCCG
AGGCACCAGCAACATGACCTATGGGACCTTCAACTTCCTCGGGGGCCGGCTGATGATCCCTAATACAG
GTATCAGCCTCCTCATCCCCCCAGATGCCATACCCCGAGGGAAGATCTATGAGATCTACCTCACGCTG
CACAAGCCGGAAGACGTGAGGTTGCCCCTAGCTGGCTGTCAGACCCTGCTGAGTCCCATCGTTAGCTG
TGGACCCCCTGGCGTCCTGCTCACCCGGCCAGTCATCCTGGCTATGGACCACTGTGGGGAGCCCAGCC
CTGACAGCTGGAGCCTGCGCCTCAAAAAGCAGTCGTGCGAGGGCAGCTGGGAGCAGGATGTGCTGCAC
CTGGGCGAGGAGGCGCCCTCCCACCTCTACTACTGCCAGCTGGAGGCCAGTGCCTGCTACGTCTTCAC
CGAGCAGCTGGGCCGCTTTGCCCTGGTGGGAGAGGCCCTCAGCGTGGCTGCCGCCAAGCGCCTCAGGC
TGCTTCTGTTTGCGCCGGTGGCCTGCACCTCCCTCGAGTACAACATCCGGGTCTACTGCCTGCATGAC
ACCCACGATGCACTCAAGGAGGTGGTGCAGCTGGAGAAGCAGCTGGGGGGACAGCTGATCCAGGAGCC
ACGGGTCCTGCACTTCAAGGACAGTTACCACAACCTGCGCCTATCCATCCACGATGTGCCCAGCTCCC
TGTGGAAGAGTAAGCTCCTTGTCAGCTACCAGGAGATCCCCTTTTATCACATCTGGAATGGCACGCAG
CGGTACTTGCACTGCACCTTCACCCTGGAGCGTGTCAGCCCCAGCACTAGTGACCTGGCCTGCAAGCT
GTGGGTGTGGCAGGTGGAGGGCGACGGGCAGAGCTTCAGCATCAACTTCAACATCACCAAGGACACAA
AAGATCCCCTTCCTCATTCGGCAGAAGATAATTTCCAGCCTGGACCCACCCTGTAGGCGGGGTGCCGA
CTGGCGGACTCTGGCCCAGAAACTCCACCTGGACAGCCATCTCAGCTTCTTTGCCTCCAAGCCCAGCC
CCACAGCCATGATCCTCAACCTGTGGGAGGCGCGGCACTTCCCCAACGGCAACCTCAGCCAGCTGGCT
GCAGCAGTGGCTGGACTGGGCCAGCCAGACGCTGGCCTCTTCACAGTGTCGGAGGCTGAGTGCTGAGG
CCGGCCAGGCCCGACACCTACACTCTCACCAGCTTTGGCACCCACCAAGGACAGGCAGAAGCCGGACA
GGGGCCCTTCCCCACACCGGGGAGA

| | | |
|---|---|---|
| NOV21p, SNP13375138 of | SEQ ID NO: 536 | MW at 99091.0kD |

| | | | |
|---|---|---|---|
| CG53908-01, Protein Sequence | SNP Pos: 651 | 899 aa | SNP Change: Lys to Arg |

MAVRPGLWPALLGIVLAAWLRGSGAQQSATVANPVPGANPDLLPHFLVEPEDVYIVKNKPVLLVCKAV
PATQIFFKCNGEWVRQVDHVIERSTDGSSGEPTMEVRINVSRQQVEKVFGLEEYWCQCVAWSSSGTTK
SQKAYIRIARLRKNFEQEPLAKEVSLEQGIVLPCRPPEGIPPAEVEWLRNEDLVDPSLDPNVYITREH
SLVVRQARLADTANYTCVAKNIVARRRSASAAVIVYVNGGWSTWTEWSVCSASCGRGWQKRSRSCTNP
APLNGGAFCEGQNVHDRTVSSLLVSVDGSWSPWSKWSACGLDCTHWRSRECSDPAPRNGGEECQGTDL
DTPNCTSDLCVHSASGPEDVALYVGLIAVAVCLVLLLLVLILVYCRKKEGLDSDVADSSILTSGFQPV
SIKPSKADNPHLLTIQPDLSTTTTYQGSLCPRQDGPSPKFQLTNGHLLSPLGGGRHTLHHSSPTSEAE
EFVSRLSTQNYFRSLPRGTSNMTYGTFNFLGGRLMIPNTGISLLIPPDAIPRGKIYEIYLTLHKPEDV
RLPLAGCQTLLSPIVSCGPPGVLLTRPVIIAMDHCGEPSPDSWSLRLKKQSCEGSWEQDVLHLGEEAP
SHLYYCQLEASACYVFTEQLGRFALVGEALSVAAAKRLRLLLFAPVACTSLEYNIRVYCLHDTHDALK
EVVQLEKQLGGQLIQEPRVLHFKDSYHNLRLSIHDVPSSLWKSKLLVSYQEIPFYHIWNGTQRYLHCT
FTLERVSPSTSDLACKLWVWQVEGDGQSFSINFNITKDTRFAELLALESEAGVPALVGPSAFKIPFLI
RQKIISSLDPPCRRGADWRTLAQKLHLDSHLSFFASKPSPTAMILNLWEARHFPNGNLSQLAAAVAGL
GQPDAGLFTVSEAEC

| | |
|---|---|
| SEQ ID NO: 537 | 2881 bp |

| | | |
|---|---|---|
| NOV21q, SNP13375494 of | ORF Start: ATG at 87 | ORF Stop: TGA at 2784 |

| | | |
|---|---|---|
| CG53908-01, DNA Sequence | SNP Pos: 2130 | SNP Change: G to C |

AGCTGGGGCTCCGGGCTGAGGCGCTAAAGCCGCCCTCCCGCCCGCGGGGCCCCGCGCCCGGCCCGCCC
GCCTGCCCGCCCGCGGCCATGGCCGTCCGGCCCGGCCTGTGGCCAGCGCTCCTGGGCATAGTCCTCGC
CGCTTGGCTCCGCGGCTCGGGTGCCCAGCAGAGTGCCACCGTGGCCAACCCAGTGCCTGGTGCCAACC
CGGACCTGCTTCCCCACTTCCTGGTGGAGCCCGAGGATGTGTACATCGTCAAGAACAAGCCAGTGCTG
CTTGTGTGCAAGGCCGTGCCCGCCACGCAGATCTTCTTCAAGTGCAACGGGGAGTGGGTGCGCCAGGT
GGACCACGTGATCGAGCGCAGCACAGACGGGAGCAGTGGTGAGCCGACCATGGAGGTCCGCATTAATG
TCTCAAGGCAGCAGGTCGAGAAGGTGTTCGGGCTGGAGGAATACTGGTGCCAGTGCGTGGCATGGAGC
TCCTCGGGCACCACCAAGAGTCAGAAGGCCTACATCCGCATAGCCAGATTGCGCAAGAACTTCGAGCA
GGAGCCGCTGGCCAAGGAGGTGTCCCTGGAGCAGGGCATCGTGCTGCCCTGCCGTCCACCGGAGGGCA
TCCCTCCAGCCGAGGTGGAGTGGCTCCGGAACGAGGACCTGGTGGACCCGTCCCTGGACCCCAATGTA
TACATCACGCGGGAGCACAGCCTGGTGGTGCGACAGGCCCGCCTTGCTGACACGGCCAACTACACCTG
CGTGGCCAAGAACATCGTGGCACGTCGCCGCAGCGCCTCCGCTGCTGTCATCGTCTACGTGAACGGTG
GGTGGTCGACGTGGACCGAGTGGTCCGTCTGCAGCGCCAGCTGTGGGCGCGGCTGGCAGAAACGGAGC
CGGAGCTGCACCAACCCGGCGCCTCTCAACGGGGGCGCTTTCTGTGAGGGGCAGAATGTCCATGACCG
CACCGTCTCCTCTCTGCTTGTCTCTGTGGACGGCAGCTGGAGCCCGTGGAGCAAGTGGTCGGCCTGTG
GGCTGGACTGCACCCACTGGCGGAGCCGTGAGTGCTCTGACCCAGCACCCCGCAACGGAGGGGAGGAG
TGCCAGGGCACTGACCTGGACACCCGCAACTGTACCAGTGACCTCTGTGTACACAGTGCTTCTGGCCC
TGAGGACGTGGCCCTCTATGTGGGCCTCATCGCCGTGGCCGTCTGCCTGGTCCTGCTGCTGCTTGTCC
TCATCCTCGTTTATTGCCGGAAGAAGGAGGGGCTGGACTCAGATGTGGCTGACTCGTCCATTCTCACC
TCAGGCTTCCAGCCCGTCAGCATCAAGCCCAGCAAAGCAGACAACCCCCATCTGCTCACCATCCAGCC
GGACCTCAGCACCACCACCACCTACCAGGGCAGTCTCTGTCCCCGGCAGGATGGGCCCAGCCCCAAGT
TCCAGCTCACCAATGGGCACCTGCTCAGCCCCCTGGGTGGCGGCCGCCACACACTGCACCACAGCTCT
AGGCACCAGCAACATGACCTATGGGACCTTCAACTTCCTCGGGGGCCGGCTGATGATCCCTAATACAG
CACAAGCCGGAAGACGTGAGGTTGCCCCTAGCTGGCTGTCAGACCCTGCTGAGTCCCATCGTTAGCTG
TGGACCCCCTGGCGTCCTGCTCACCCGGCCAGTCATCCTGGCTATGGACCACTGTGGGGAGCCCAGCC
CTGACAGCTGGAGCCTGCGCCTCAAAAAGCAGTCGTGCGAGGGCAGCTGGGAGCAGGATGTGCTGCAC
CTGGGCGAGGAGGCGCCCTCCCACCTCTACTACTGCCAGCTGGAGGCCAGTGCCTGCTACGTCTTCAC
CGAGCAGCTGGGCCGCTTTGCCCTGGTGGGAGAGGCCCTCAGCGTGGCTGCCGCCAAGCGCCTCAAGC
TGCTTCTGTTTGCGCCGGTGGCCTGCACCTCCCTCGAGTACAACATCCGGGTCTACTGCCTGCATGAC
ACCCACGATGCACTCAAGGAGCTGGTGCAGCTGGAGAAGCAGCTGGGGGGACAGCTGATCCAGGAGCC
ACGGGTCCTGCACTTCAAGGACAGTTACCACAACCTGCGCCTATCCATCCACGATGTGCCCAGCTCCC
CGGTACTTGCACTGCACCTTCACCCTGGAGCGTGTCAGCCCCAGCACTAGTGACCTGGCCTGCAAGCT
GTGGGTGTGGCAGGTGGAGGGCGACGGGCAGAGCTTCAGCATCAACTTCAACATCACCAAGGACACAA
GGTTTGCTGAGCTGCTGGCTCTGGAGAGTGAAGCGGGGGTCCCAGCCCTGGTGGGCCCCAGTGCCTTC
AAGATCCCCTTCCTCATTCGGCAGAAGATAATTTCCAGCCTGGACCCACCCTGTAGGCGGGGTGCCGA
CTGGCGGACTCTGGCCCAGAAACTCCACCTGGACAGCCATCTCAGCTTCTTTGCCTCCAAGCCCAGCC
CCACAGCCATGATCCTCAACCTGTGGGAGGCGCGGCACTTCCCCAACGGCAACCTCAGCCAGCTGGCT
GCAGCAGTGGCTGGACTGGGCCAGCCAGACGCTGGCCTCTTCACAGTGTCGGAGGCTGAGTGCTGAGG
CCGGCCAGGCCCGACACCTACACTCTCACCAGCTTTGGCACCCACCAAGGACAGGCAGAAGCCGGACA
GGGGCCCTTCCCCACACCGGGGAGA

| | | |
|---|---|---|
| NOV21q, SNP13375494 of | SEQ ID NO: 538 | MW at 99077.0kD |

| | | | |
|---|---|---|---|
| CG53908-01, Protein Sequence | SNP Pos: 682 | 899 aa | SNP Change: Val to Leu |

MAVRPGLWPALLGIVLAAWLRGSGAQQSATVANPVPGANPDLLPHFLVEPEDVYIVKNKPVLLVCKAV
PATQIFFKCNGEWVRQVDHVIERSTDGSSGEPTMEVRINVSRQQVEKVFGLEEYWCQCVAWSSSGTTK
SQKAYIRIARLRKNFEQEPLAKEVSLEQGIVLPCRPPEGIPPAEVEWLRNEDLVDPSLDPNVYITREH
SLVVRQARLADTANYTCVAKNTVARRRSASAAVIVYVNGGWSTWTEWSVCSASCGRGWQKRSRSCTNP
APLNGGAFCEGQNVHDRTVSSLLVSVDGSWSPWSKWSACGLDCTHWRSRECSDPAPRNGGEECQGTDL
DTRNCTSDLCVHSASGPEDVALYVGLIAVAVCLVLLLLVLILVYCRKKEGLDSDVADSSILTSGFQPV
SIKPSKADNPHLLTIQPDLSTTTTYQGSLCPRQDGPSPKFQLTNGHLLSPLGGGRHTLHHSSPTSEAE
EFVSRLSTQNYFRSLPRGTSNMTYGTFNFLGGRLMIPNTGISLLIPPDAIPRGKIYEIYLTLHKPEDV
RLPLAGCQTLLSPIVSCGPPGVLLTRPVILAMDHCGEPSPDSWSLRLKKQSCEGSWEQDVLHLGEEAP
SHLYYCQLEASACYVFTEQLGRFALVGEALSVAAAKRLKLLLFAPVACTSLEYNIRVYCLHDTHDALK
ELVQLEKQLGGQLIQEPRVLHFKDSYHNLRLSIHDVPSSLWKSKLLVSYQEIPFYHIWNGTQRYLHCT
FTLERVSPSTSDLACKLWVWQVEGDGQSFSINFNITKDTRFAELLALESEAGVPALVGPSAFKIPFLI
RQKIISSLDPPCRRGADWRTLAQKLHLDSHLSFFASKPSPTAMILNLWEARHFPNGNLSQLAAAVAGL
GQPDAGLFTVSEAEC

| | |
|---|---|
| SEQ ID NO: 539 | 2881 bp |

| | | |
|---|---|---|
| NOV21r, SNP13375495 of | ORF Start: ATG at 87 | ORF Stop: TGA at 2784 |

| | | |
|---|---|---|
| CG53908-01, DNA Sequence | SNP Pos: 2305 | SNP Change: G to A |

AGCTGGGGCTCCGGGCTGAGGCGCTAAAGCCGCCCTCCCGCCCGCGGGGCCCCGCGCCCGGCCCGCCC
GCCTGCCCGCCCGCGGCCATGGCCGTCCGGCCCGGCCTGTGGCCAGCGCTCCTGGGCATAGTCCTCGC
CGCTTGGCTCCGCGGCTCGGGTGCCCAGCAGAGTGCCACCGTGGCCAACCCAGTGCCTGGTGCCAACC
CGGACCTGCTTCCCCACTTCCTGGTGGAGCCCGAGGATGTGTACATCGTCAAGAACAAGCCAGTGCTG
CTTGTGTGCAAGGCCGTGCCCGCCACGCAGATCTTCTTCAAGTGCAACGGGGAGTGGGTGCGCCAGGT
GGACCACGTGATCGAGCGCAGCACAGACGGGAGCAGTGGTGAGCCGACCATGGAGGTCCGCATTAATG
TCTCAAGGCAGCAGGTCGAGAAGGTGTTCGGGCTGGAGGAATACTGGTGCCAGTGCGTGGCATGGAGC
TCCTCGGGCACCACCAAGAGTCAGAAGGCCTACATCCGCATAGCCAGATTGCGCAAGAACTTCGAGCA
GGAGCCGCTGGCCAAGGAGGTGTCCCTGGAGCAGGGCATCGTGCTGCCCTGCCGTCCACCGGAGGGCA
TCCCTCCAGCCGAGGTGGAGTGGCTCCGGAACGAGGACCTGGTGGACCCGTCCCTGGACCCCAATGTA
TACATCACGCGGGAGCACAGCCTGGTGGTGCGACAGGCCCGCCTTGCTGACACGGCCAACTACACCTG
CGTGGCCAAGAACATCGTGGCACGTCGCCGCAGCGCCTCCGCTGCTGTCATCGTCTACGTGAACGGTG
GGTGGTCGACGTGGACCGAGTGGTCCGTCTGCAGCGCCAGCTGTGGGCGCGGCTGGCAGAAACGGAGC
CGGAGCTGCACCAACCCGGCGCCTCTCAACGGGGGCGCTTTCTGTGAGGGGCAGAATGTCCATGACCG
CACCGTCTCCTCTCTGCTTGTCTCTGTGGACGGCAGCTGGAGCCCGTGGAGCAAGTGGTCGGCCTGTG
GGCTGGACTGCACCCACTGGCGGAGCCGTGAGTGCTCTGACCCAGCACCCCGCAACGGAGGGGAGGAG
TGCCAGGGCACTGACCTGGACACCCGCAACTGTACCAGTGACCTCTGTGTACACAGTGCTTCTGGCCC
TCATCCTCGTTTATTGCCGGAAGAAGGAGGGGCTGGACTCAGATGTGGCTGACTCGTCCATTCTCACC
TCAGGCTTCCAGCCCGTCAGCATCAAGCCCAGCAAAGCAGACAACCCCCATCTGCTCACCATCCAGCC
GGACCTCAGCACCACCACCACCTACCAGGGCAGTCTCTGTCCCCGGCAGGATGGGCCCAGCCCCAAGT
TCCAGCTCACCAATGGGCACCTGCTCAGCCCCCTGGGTGGCGGCCGCCACACACTGCACCACAGCTCT
CCCACCTCTGAGGCCGAGGAGTTCGTCTCCCGCCTCTCCACCCAGAACTACTTCCGCTCCCTGCCCCG
AGGCACCAGCAACATGACCTATGGGACCTTCAACTTCCTCGGGGGCCGGCTGATGATCCCTAATACAG
GTATCAGCCTCCTCATCCCCCCAGATGCCATACCCCGAGGGAAGATCTATGAGATCTACCTCACGCTG
CACAAGCCGGAAGACGTGAGGTTGCCCCTAGCTGGCTGTCAGACCCTGCTGAGTCCCATCGTTAGCTG
TGGACCCCCTGGCGTCCTGCTCACCCGGCCAGTCATCCTGGCTATGGACCACTGTGGGGAGCCCAGCC
CTGACAGCTGGAGCCTGCGCCTCAAAAAGCAGTCGTGCGAGGGCAGCTGGGAGCAGGATGTGCTGCAC
CTGGGCGAGGAGGCGCCCTCCCACCTCTACTACTGCCAGCTGGAGGCCAGTGCCTGCTACGTCTTCAC
CGAGCAGCTGGGCCGCTTTGCCCTGGTGGGAGAGGCCCTCAGCGTGGCTGCCGCCAAGCGCCTCAAGC
TGCTTCTGTTTGCGCCGGTGGCCTGCACCTCCCTCGAGTACAACATCCGGGTCTACTGCCTGCATGAC
ACCCACGATGCACTCAAGGAGGTGGTGCAGCTGGAGAAGCAGCTGGGGGGACAGCTGATCCAGGAGCC
ACGGGTCCTGCACTTCAAGGACAGTTACCACAACCTGCGCCTATCCATCCACGATGTGCCCAGCTCCC
TGTGGAAGAGTAAGCTCCTTGTCAGCTACCAGGAGATCCCCTTTTATCACATCTGGAATGACACGCAG
CGGTACTTGCACTGCACCTTCACCCTGGAGCGTGTCAGCCCCAGCACTAGTGACCTGGCCTGCAAGCT
GTGGGTGTGGCAGGTGGAGGGCGACGGGCAGAGCTTCAGCATCAACTTCAACATCACCAAGGACACAA
AAGATCCCCTTCCTCATTCGGCAGAAGATAATTTCCAGCCTGGACCCACCCTGTAGGCGGGGTGCCGA
CTGGCGGACTCTGGCCCAGAAACTCCACCTGGACAGCCATCTCAGCTTCTTTGCCTCCAAGCCCAGCC
CCACAGCCATGATCCTCAACCTGTGGGAGGCGCGGCACTTCCCCAACGGCAACCTCAGCCAGCTGGCT
GCAGCAGTGGCTGGACTGGGCCAGCCAGACGCTGGCCTCTTCACAGTGTCGGAGGCTGAGTGCTGAGG
CCGGCCAGGCCCGACACCTACACTCTCACCAGCTTTGGCACCCACCAAGGACAGGCAGAAGCCGGACA
GGGGCCCTTCCCCACACCGGGGAGA

| | | |
|---|---|---|
| NOV21r, SNP13375495 of | SEQ ID NO: 540 | MW at 99121.0kD |

| | | | |
|---|---|---|---|
| CG53908-01, Protein Sequence | SNP Pos: 740 | 899 aa | SNP Change: Gly to Asp |

MAVRPGLWPALLGIVLAAWLRGSGAQQSATVANPVPGANPDLLPHFLVEPEDVYIVKNKPVLLVCKAV
PATQIFFKCNGEWVRQVDHVIERSTDGSSGEPTMEVRINVSRQQVEKVFGLEEYWCQCVAWSSSGTTK
SQKAYIRIARLRKNFEQEPLAKEVSLEQGIVLPCRPPEGIPPAEVEWLRNEDLVDPSLDPNVYITREH
SLVVRQARLADTANYTCVAKNIVARPRSASAAVIVYVNGGWSTWTEWSVCSASCGRGWQKRSRSCTNP
APLNGGAFCEGQNVHDRTVSSLLVSVDGSWSPWSKWSACGLDCTHWRSRECSDPAPRNGGEECQGTDL
DTRNCTSDLCVHSASGPEDVALYVGLIAVAVCLVLLLLVLILVYCRKKEGLDSDVADSSILTSGFQPV
SIKPSKADNPHLLTIQPDLSTTTTYQGSLCPRQDGPSPKFQLTNGHLLSPLGGGRHTLHHSSPTSEAE
EFVSRLSTQNYFRSLPRGTSNMTYGTFNFLGGRLMIPNTGISLLIPPDAIPRGKIYEIYLTLHKPEDV
RLPLAGCQTLLSPIVSCGPPGVLLTRPVILAMDHCGEPSPDSWSLRLKKQSCEGSWEQDVLHLGEEAP
SHLYYCQLEASACYVFTEQLGRFALVGEALSVAAAKRLKLLLFAPVACTSLEYNIRVYCLHDTHDALK
EVVQLEKQLGGQLIQEPRVLHFKDSYHNLRLSIHDVPSSLWKSKLLVSYQEIPFYHIWNDTQRYLHCT
FTLERVSPSTSDLACKLWVWQVEGDGQSFSINFNITKDTRFAELLALESEAGVPALVGPSAFKIPFLI
RQKIISSLDPPCRRGADWRTLAQKLHLDSHLSFFASKPSPTAMILNLWEARHFPNGNLSQLAAAVAGL
GQPDAGLFTVSEAEC

| | |
|---|---|
| SEQ ID NO: 541 | 2881 bp |

| | | |
|---|---|---|
| NOV21s, SNP13375140 of | ORF Start: ATG at 87 | ORF Stop: TGA at 2784 |

| | | |
|---|---|---|
| CG53908-01, DNA Sequence | SNP Pos: 2325 | SNP Change: T to C |

AGCTGGGGCTCCGGGCTGAGGCGCTAAAGCCGCCCTCCCGCCCGCGGGGCCCCGCGCCCGGCCCGCCC
GCCTGCCCGCCCGCGGCCATGGCCGTCCGGCCCGGCCTGTGGCCAGCGCTCCTGGGCATAGTCCTCGC
CGCTTGGCTCCGCGGCTCGGGTGCCCAGCAGAGTGCCACCGTGGCCAACCCAGTGCCTGGTGCCAACC
CGGACCTGCTTCCCCACTTCCTGGTGGAGCCCGAGGATGTGTACATCGTCAAGAACAAGCCAGTGCTG
CTTGTGTGCAAGGCCGTGCCCGCCACGCAGATCTTCTTCAAGTGCAACGGGGAGTGGGTGCGCCAGGT
GGACCACGTGATCGAGCGCAGCACAGACGGGAGCAGTGGTGAGCCGACCATGGAGGTCCGCATTAATG
TCTCAAGGCAGCAGGTCGAGAAGGTGTTCGGGCTGGAGGAATACTGGTGCCAGTGCGTGGCATGGAGC
TCCTCGGGCACCACCAAGAGTCAGAAGGCCTACATCCGCATAGCCAGATTGCGCAAGAACTTCGAGCA
GGAGCCGCTGGCCAAGGAGGTGTCCCTGGAGCAGGGCATCGTGCTGCCCTGCCGTCCACCGGAGGGCA
TCCCTCCAGCCGAGGTGGAGTGGCTCCGGAACGAGGACCTGGTGGACCCGTCCCTGGACCCCAATGTA
TACATCACGCGGGAGCACAGCCTGGTGGTGCGACAGGCCCGCCTTGCTGACACGGCCAACTACACCTG
CGTGGCCAAGAACATCGTGGCACGTCGCCGCAGCGCCTCCGCTGCTGTCATCGTCTACGTGAACGGTG
GGTGGTCGACGTGGACCGAGTGGTCCGTCTGCAGCGCCAGCTGTGGGCGCGGCTGGCAGAAACGGAGC
CGGAGCTGCACCAACCCGGCGCCTCTCAACGGGGGCGCTTTCTGTGAGGGGCAGAATGTCCATGACCG
CACCGTCTCCTCTCTGCTTGTCTCTGTGGACGGCAGCTGGAGCCCGTGGAGCAAGTGGTCGGCCTGTG
TGCCAGGGCACTGACCTGGACACCCGCAACTGTACCAGTGACCTCTGTGTACACAc3TGCTTCTGGCCC
TGAGGACGTGGCCCTCTATGTGGGCCTCATCGCCGTGGCCGTCTGCCTGGTCCTGCTGCTGCTTGTCC
TCATCCTCGTTTATTGCCGGAAGAAGGAGGGGCTGGACTCAGATGTGGCTGACTCGTCCATTCTCACC
TCAGGCTTCCAGCCCGTCAGCATCAAGCCCAGCAAAGCAGACAACCCCCATCTGCTCACCATCCAGCC
GGACCTCAGCACCACCACCACCTACCAGGGCAGTCTCTGTCCCCGGCAGGATGGGCCCAGCCCCAAGT
TCCAGCTCACCAATGGGCACCTGCTCAGCCCCCTGGGTGGCGGCCGCCACACACTGCACCACAGCTCT
CCCACCTCTGAGGCCGAGGAGTTCGTCTCCCGCCTCTCCACCCAGAACTACTTCCGCTCCCTGCCCCG
AGGCACCAGCAACATGACCTATGGGACCTTCAACTTCCTCGGGGGCCGGCTGATGATCCCTAATACAG
GTATCAGCCTCCTCATCCCCCCAGATGCCATACCCCGAGGGAAGATCTATGAGATCTACCTCACGCTG
CACAAGCCGGAAGACGTGAGGTTGCCCCTAGCTGGCTGTCAGACCCTGCTGAGTCCCATCGTTAGCTG
TGGACCCCCTGGCGTCCTGCTCACCCGGCCAGTCATCCTGGCTATGGACCACTGTGGGGAGCCCAGCC
CTGACAGCTGGAGCCTGCGCCTCAAAAAGCAGTCGTGCGAGGGCAGCTGGGAGCAGGATGTGCTGCAC
CTGGGCGAGGAGGCGCCCTCCCACCTCTACTACTGCCAGCTGGAGGCCAGTGCCTGCTACGTCTTCAC
CGAGCAGCTGGGCCGCTTTGCCCTGGTGGGAGAGGCCCTCAGCGTGGCTGCCGCCAAGCGCCTCAAGC
TGCTTCTGTTTGCGCCGGTGGCCTGCACCTCCCTCGAGTACAACATCCGGGTCTACTGCCTGCATGAC
ACCCACGATGCACTCAAGGAGGTGGTGCAGCTGGAGAAGCAGCTGGGGGGACAGCTGATCCAGGAGCC
ACGGGTCCTGCACTTCAAGGACAGTTACCACAACCTGCGCCTATCCATCCACGATGTGCCCAGCTCCC
TGTGGAAGAGTAAGCTCCTTGTCAGCTACCAGGAGATCCCCTTTTATCACATCTGGAATGGCACGCAG
CGGTACTTGCACCGCACCTTCACCCTGGAGCGTGTCAGCCCCAGCACTAGTGACCTGGCCTGCAAGCT
GTGGGTGTGGCAGGTGGAGGGCGACGGGCAGAGCTTCAGCATCAACTTCAACATCACCAAGGACACAA
AAGATCCCCTTCCTCATTCGGCAGAAGATAATTTCCAGCCTGGACCCACCCTGTAGGCGGGGTGCCGA
CTGGCGGACTCTGGCCCAGAAACTCCACCTGGACAGCCATCTCAGCTTCTTTGCCTCCAAGCCCAGCC
CCACAGCCATGATCCTCAACCTGTGGGAGGCGCGGCACTTCCCCAACGGCAACCTCAGCCAGCTGGCT
GCAGCAGTGGCTGGACTGGGCCAGCCAGACGCTGGCCTCTTCACAGTGTCGGAGGCTGAGTGCTGAGG
CCGGCCAGGCCCGACACCTACACTCTCACCAGCTTTGGCACCCACCAAGGACAGGCAGAAGCCGGACA
GGGGCCCTTCCCCACACCGGGGAGA

| | | |
|---|---|---|
| NOV21s, SNP13375140 of | SEQ ID NO: 542 | MW at 99116.0kD |

| | | | |
|---|---|---|---|
| CG53908-01, Protein Sequence | SNP Pos: 747 | 899 aa | SNP Change: Cys to Arg |

MAVRPGLWPALLGIVLAAWLRGSGAQQSATVANPVPGANPDLLPHFLVEPEDVYIVKNKPVLLVCKAV
PATQIFFKCNGEWVRQVDHVIERSTDGSSGEPTMEVRINVSRQQVEKVFGLEEYWCQCVAWSSSGTTK
SQKAYIRIARLRKNFEQEPLAKEVSLEQGIVLPCRPPEGIPPAEVEWLRNEDLVDPSLDPNVYITREH
SLVVRQARLADTANYTCVAKNIVARRRSASAAVIVYVNGGWSTWTEWSVCSASCGRGWQKRSRSCTNP
APLNGGAFCEGQNVHDRTVSSLLVSVDGSWSPWSKWSACGLDCTHWRSRECSDPAPRNGGEECQGTDL
DTRNCTSDLCVHSASGPEDVALYVGLIAVAVCLVLLLLVLILVYCRKKEGLDSDVADSSILTSGFQPV
SIKPSKADNPHLLTIQPDLSTTTTYQGSLCPRQDGPSPKFQLTNGHLLSPLGGGRHTLHHSSPTSFAE
EFVSRLSTQNYFRSLPRGTSNMTYGTFNFLGGRLMIPNTGISLLIPPDAIPRGKIYEIYLTLHKPEDV
RLPLAGCQTLLSPIVSCGPPGVLLTRPVILAMDHCGEPSPDSWSLRLKKQSCEGSWEQDVLHLGEEAP
EVVQLEKQLGGQLIQEPRVLHFKDSYHNLRLSIHDVPSSLWKSKLLVSYQEIPFYHIWNGTQRYLHRT
FTLERVSPSTSDLACKLWVWQVEGDGQSFSINFNITKDTRFAELLALESEAGVPALVGPSAFKIPFLI
RQKIISSLDPPCRRGADWRTLAQKLHLDSHLSFFASKPSPTAMILNLWEARHFPNGNLSQLAAAVAGL
GQPDAGLFTVSEAEC

| | |
|---|---|
| SEQ ID NO: 543 | 2881 bp |

| | | |
|---|---|---|
| NOV21t, SNP13375139 of | ORF Start: ATG at 87 | ORF Stop: TGA at 2784 |

| | | |
|---|---|---|
| CG53908-01, DNA Sequence | SNP Pos: 2404 | Change: A to G |

GCCTGCCCGCCCGCGGCCATGGCCGTCCGGCCCGGCCTGTGGCCAGCGCTCCTGGGCATAGTCCTCGC
CGGACCTGCTTCCCCACTTCCTGGTGGAGCCCGAGGATGTGTACATCGTCAAGAACAAGCCAGTGCTG
CTTGTGTGCAAGGCCGTGCCCGCCACGCAGATCTTCTTCAAGTGCAACGGGGAGTGGGTGCGCCAGGT
GGACCACGTGATCGAGCGCAGCACAGACGGGAGCAGTGGTGAGCCGACCATGGAGGTCCGCATTAATG
TCTCAAGGCAGCAGGTCGAGAAGGTGTTCGGGCTGGAGGAATACTGGTGCCAGTGCGTGGCATGGAGC
TCCTCGGGCACCACCAAGAGTCAGAAGGCCTACATCCGCATAGCCAGATTGCGCAAGAACTTCGAGCA
GGAGCCGCTGGCCAAGGAGGTGTCCCTGGAGCAGGGCATCGTGCTGCCCTGCCGTCCACCGGAGGGCA
TCCCTCCAGCCGAGGTGGAGTGGCTCCGGAACGAGGACCTGGTGGACCCGTCCCTGGACCCCAATGTA
TACATCACGCGGGAGCACAGCCTGGTGGTGCGACAGGCCCGCCTTGCTGACACGGCCAACTACACCTG
CGTGGCCAAGAACATCGTGGCACGTCGCCGCAGCGCCTCCGCTGCTGTCATCGTCTACGTGAACGGTG
GGTGGTCGACGTGGACCGAGTGGTCCGTCTGCAGCGCCAGCTGTGGGCGCGGCTGGCAGAAACGGAGC
CGGAGCTGCACCAACCCGGCGCCTCTCAACGGGGGCGCTTTCTGTGAGGGGCAGAATGTCCATGACCG
CACCGTCTCCTCTCTGCTTGTCTCTGTGGACGGCAGCTGGAGCCCGTGGAGCAAGTGGTCGGCCTGTG
GGCTGGACTGCACCCACTGGCGGAGCCGTGAGTGCTCTGACCCAGCACCCCGCAACGGAGGGGAGGAG
TGCCAGGGCACTGACCTGGACACCCGCAACTGTACCAGTGACCTCTGTGTACACAGTGCTTCTGGCCC
TGAGGACGTGGCCCTCTATGTGGGCCTCATCGCCGTGGCCGTCTGCCTGGTCCTGCTGCTGCTTGTCC
TCATCCTCGTTTATTGCCGGAAGAAGGAGGGGCTGGACTCAGATGTGGCTGACTCGTCCATTCTCACC
TCAGGCTTCCAGCCCGTCAGCATCAAGCCCAGCAAAGCAGACAACCCCCATCTGCTCACCATCCAGCC
GGACCTCAGCACCACCACCACCTACCAGGGCAGTCTCTGTCCCCGGCAGGATGGGCCCAGCCCCAAGT
TCCAGCTCACCAATGGGCACCTGCTCAGCCCCCTGGGTGGCGGCCGCCACACACTGCACCACAGCTCT
AGGCACCAGCAACATGACCTATGGGACCTTCAACTTCCTCGGGGGCCGGCTGATGATCCCTAATACAG
GTATCAGCCTCCTCATCCCCCCAGATGCCATACCCCGAGGGAAGATCTATGAGATCTACCTCACGCTG
CACAAGCCGGAAGACGTGAGGTTGCCCCTAGCTGGCTGTCAGACCCTGCTGAGTCCCATCGTTAGCTG
TGGACCCCCTGGCGTCCTGCTCACCCGGCCAGTCATCCTGGCTATGGACCACTGTGGGGAGCCCAGCC
CTGACAGCTGGAGCCTGCGCCTCAAAAAGCAGTCGTGCGAGGGCAGCTGGGAGCAGGATGTGCTGCAC
CTGGGCGAGGAGGCGCCCTCCCACCTCTACTACTGCCAGCTGGAGGCCAGTGCCTGCTACGTCTTCAC
CGAGCAGCTGGGCCGCTTTGCCCTGGTGGGAGAGGCCCTCAGCGTGGCTGCCGCCAAGCGCCTCAAGC
TGCTTCTGTTTGCGCCGGTGGCCTGCACCTCCCTCGAGTACAACATCCGGGTCTACTGCCTGCATGAC
ACCCACGATGCACTCAAGGAGGTGGTGCAGCTGGAGAAGCAGCTGGGGGGACAGCTGATCCAGGAGCC
ACGGGTCCTGCACTTCAAGGACAGTTACCACAACCTGCGCCTATCCATCCACGATGTGCCCAGCTCCC
TGTGGAAGAGTAAGCTCCTTGTCAGCTACCAGGAGATCCCCTTTTATCACATCTGGAATGGCACGCAG
CGGTACTTGCACTGCACCTTCACCCTGGAGCGTGTCAGCCCCAGCACTAGTGACCTGGCCTGCAAGCT
GTGGGTGTGGCAGGTGGAGGGCGGCGGGCAGAGCTTCAGCATCAACTTCAACATCACCAAGGACACAA
GGTTTGCTGAGCTGCTGGCTCTGGAGAGTGAAGCGGGGGTCCCAGCCCTGGTGGGCCCCAGTGCCTTC
CTGGCGGACTCTGGCCCAGAAACTCCACCTGGACAGCCATCTCAGCTTCTTTGCCTCCAAGCCCAGCC
CCACAGCCATGATCCTCAACCTGTGGGAGGCGCGGCACTTCCCCAACGGCAACCTCAGCCAGCTGGCT
GCAGCAGTGGCTGGACTGGGCCAGCCAGACGCTGGCCTCTTCACAGTGTCGGAGGCTGAGTGCTGAGG
CCGGCCAGGCCCGACACCTACACTCTCACCAGCTTTGGCACCCACCAAGGACAGGCAGAAGCCGGACA
GGGGCCCTTCCCCACACCGGGGAGA

| | | |
|---|---|---|
| NOV21t, SNP13375139 of | SEQ ID NO: 544 | MW at 99005.0kD |

| | | | |
|---|---|---|---|
| CG53908-01, Protein Sequence | SNP Pos: 773 | 899 aa | SNP Change: Asp to Gly |

MAVRPGLWPALLGIVLAAWLRGSGAQQSATVANPVPGANPDLLPHFLVEPEDVYTVKNKPVLLVCKAV
PATQIFFKCNGEWVRQVDHVIERSTDGSSGEPTMEVRINVSRQQVEKVFGLEEYWCQCVAWSSSGTTK
SQKAYIRIARLRKNFEQEPLAKEVSLEQGIVLPCRPPEGIPPAEVEWLRNEDLVDPSLDPNVYITREH
SLVVRQARLADTANYTCVAKNIVARRRSASAAVIVYVNGGWSTWTEWSVCSASCGRGWQKRSRSCTNP
APLNGGAFCEGQNVHDRTVSSLLVSVDGSWSPWSKWSACGLDCTHWRSRECSDPAPRNGGEECQGTDL
DTRNCTSDLCVHSASGPEDVALYVGLIAVAVCLVLLLLVLILVYCRKKEGLDSDVADSSILTSGFQPV
SIKPSKADNPHLLTIQPDLSTTTTYQGSLCPRQDGPSPKFQLTNGHLLSPLGGGRHTLHHSSPTSEAE
EFVSRLSTQNYFRSLPRGTSNMTYGTFNFLGGRLMIPNTGISLLIPPDAIPRGKIYEIYLTLHKPEDV
RLPLAGCQTLLSPIVSCGPPGVLLTRPVILAMDHCGEPSPDSWSLRLKKQSCEGSWEQDVLHLGEEAP
SHLYYCQLEASACYVFTEQLGRFALVGEALSVAAAKRLKLLLFAPVACTSLEYNIRVYCLHDTHDALK
EVVQLEKQLGGQLIQEPRVLHFKDSYHNLRLSIHDVPSSLWKSKLLVSYQEIPFYHIWNGTQRYLHCT
FTLERVSPSTSDLACKLWVWQVEGGGQSFSINFNITKDTRFAELLALESEAGVPALVGPSAFKIPFLI
RQKIISSLDPPCRRGADWRTLAQKLHLDSHLSFFASKPSPTAMILNLWEARHFPNGNLSQLAAAVAGL
GQPDAGLFTVSEAEC

| | |
|---|---|
| SEQ ID NO: 545 | 2881 bp |

| | | |
|---|---|---|
| NOV21u, SNP13375496 of | ORF Start: ATG at 87 | ORF Stop: TGA at 2784 |

| | | |
|---|---|---|
| CG53908-01, DNA Sequence | SNP Pos: 2676 | SNP Change: T to C |

AGCTGGGGCTCCGGGCTGAGGCGCTAAAGCCGCCCTCCCGCCCGCGGGGCCCCGCGCCCGGCCCGCCC
GCCTGCCCGCCCGCGGCCATGGCCGTCCGGCCCGGCCTGTGGCCAGCGCTCCTGGGCATAGTCCTCGC
CGCTTGGCTCCGCGGCTCGGGTGCCCAGCAGAGTGCCACCGTGGCCAACCCAGTGCCTGGTGCCAACC
CGGACCTGCTTCCCCACTTCCTGGTGGAGCCCGAGGATGTGTACATCGTCAAGAACAAGCCAGTGCTG
CTTGTGTGCAAGGCCGTGCCCGCCACGCAGATCTTCTTCAAGTGCAACGGGGAGTGGGTGCGCCAGGT
TCTCAAGGCAGCAGGTCGAGAAGGTGTTCGGGCTGGAGGAATACTGGTGCCAGTGCGTGGCATGGAGC
TCCTCGGGCACCACCAAGAGTCAGAAGGCCTACATCCGCATAGCCAGATTGCGCAAGAACTTCGAGCA
GGAGCCGCTGGCCAAGGAGGTGTCCCTGGAGCAGGGCATCGTGCTGCCCTGCCGTCCACCGGAGGGCA
TCCCTCCAGCCGAGGTGGAGTGGCTCCGGAACGAGGACCTGGTGGACCCGTCCCTGGACCCCAATGTA
TACATCACGCGGGAGCACAGCCTGGTGGTGCGACAGGCCCGCCTTGCTGACACGGCCAACTACACCTG
CGTGGCCAAGAACATCGTGGCACGTCGCCGCAGCGCCTCCGCTGCTGTCATCGTCTACGTGAACGGTG
GGTGGTCGACGTGGACCGAGTGGTCCGTCTGCAGCGCCAGCTGTGGGCGCGGCTGGCAGAAACGGAGC
CGGAGCTGCACCAACCCGGCGCCTCTCAACGGGGGCGCTTTCTGTGAGGGGCAGAATGTCCATGACCG
CACCGTCTCCTCTCTGCTTGTCTCTGTGGACGGCAGCTGGAGCCCGTGGAGCAAGTGGTCGGCCTGTG
GGCTGGACTGCACCCACTGGCGGAGCCGTGAGTGCTCTGACCCAGCACCCCGCAACGGAGGGGAGGAG
TGCCAGGGCACTGACCTGGACACCCGCAACTGTACCAGTGACCTCTGTGTACACAGTGCTTCTGGCCC
TGAGGACGTGGCCCTCTATGTGGGCCTCATCGCCGTGGCCGTCTGCCTGGTCCTGCTGCTGCTTGTCC
TCATCCTCGTTTATTGCCGGAAGAAGGAGGGGCTGGACTCAGATGTGGCTGACTCGTCCATTCTCACC
TCCAGCTCACCAATGGGCACCTGCTCAGCCCCCTGGGTGGCGGCCGCCACACACTGCACCACAGCTCT
CCCACCTCTGAGGCCGAGGAGTTCGTCTCCCGCCTCTCCACCCAGAACTACTTCCGCTCCCTGCCCCG
AGGCACCAGCAACATGACCTATGGGACCTTCAACTTCCTCGGGGGCCGGCTGATGATCCCTAATACAG
GTATCAGCCTCCTCATCCCCCCAGATGCCATACCCCGAGGGAAGATCTATGAGATCTACCTCACGCTG
TGGACCCCCTGGCGTCCTGCTCACCCGGCCAGTCATCCTGGCTATGGACCACTGTGGGGAGCCCAGCC
CTGACAGCTGGAGCCTGCGCCTCAAAAAGCAGTCGTGCGAGGGCAGCTGGGAGCAGGATGTGCTGCAC
CTGGGCGAGGAGGCGCCCTCCCACCTCTACTACTGCCAGCTGGAGGCCAGTGCCTGCTACGTCTTCAC
CGAGCAGCTGGGCCGCTTTGCCCTGGTGGGAGAGGCCCTCAGCGTGGCTGCCGCCAAGCGCCTCAAGC
TGCTTCTGTTTGCGCCGGTGGCCTGCACCTCCCTCGAGTACAACATCCGGGTCTACTGCCTGCATGAC
ACCCACGATGCACTCAAGGAGGTGGTGCAGCTGGAGAAGCAGCTGGGGGGACAGCTGATCCAGGAGCC
ACGGGTCCTGCACTTCAAGGACAGTTACCACAACCTGCGCCTATCCATCCACGATGTGCCCAGCTCCC
TGTGGAAGAGTAAGCTCCTTGTCAGCTACCAGGAGATCCCCTTTTATCACATCTGGAATGGCACGCAG
CGGTACTTGCACTGCACCTTCACCCTGGAGCGTGTCAGCCCCAGCACTAGTGACCTGGCCTGCAAGCT
GTGGGTGTGGCAGGTGGAGGGCGACGGGCAGAGCTTCAGCATCAACTTCAACATCACCAAGGACACAA
GGTTTGCTGAGCTGCTGGCTCTGGAGAGTGAAGCGGGGGTCCCAGCCCTGGTGGGCCCCAGTGCCTTC
AAGATCCCCTTCCTCATTCGGCAGAAGATAATTTCCAGCCTGGACCCACCCTGTAGGCGGGGTGCCGA
CTGGCGGACTCTGGCCCAGAAACTCCACCTGGACAGCCATCTCAGCTTCTTTGCCTCCAAGCCCAGCC
CCACAGCCATGATCCTCAACCTGCGGGAGGCGCGGCACTTCCCCAACGGCAACCTCAGCCAGCTGGCT
GCAGCAGTGGCTGGACTGGGCCAGCCAGACGCTGGCCTCTTCACAGTGTCGGAGGCTGAGTGCTGAGG
CCGGCCAGGCCCGACACCTACACTCTCACCAGCTTTGGCACCCACCAAGGACAGGCAGAAGCCGGACA
GGGGCCCTTCCCCACACCGGGGAGA

| | | |
|---|---|---|
| NOV21u, SNP13375496 of | SEQ ID NO: 546 | MW at 99032.9kD |

| | | | |
|---|---|---|---|
| CG53908-01, Protein Sequence | SNP Pos: 864 | 899 aa | SNP Change: Trp to Arg |

MAVRPGLWPALLGIVLAAWLRGSGAQQSATVANPVPGANPDLLPHFLVEPEDVYIVKNKPVLLVCKAV
PATQIFFKCNGEWVRQVDHVTERSTDGSSGEPTMEVRINVSRQQVEKVFGLEEYWCQCVAWSSSGTTK
SQKAYIRIARLRKNFEQEPLAKEVSLEQGIVLPCRPPEGIPPAEVEWLRNEDLVDPSLDPNVYITREH
SLVVRQARLADTANYTCVAKNIVARRRSASAAVIVYVNGGWSTWTEWSVCSASCGRGWQKRSRSCTNP
APLNGGAFCEGQNVHDRTVSSLLVSVDGSWSPWSKWSACGLDCTHWRSRECSDPAPRNGGEECQGTDL
DTRNCTSDLCVHSASGPEDVALYVGLIAVAVCLVLLLLVLILVYCRKKEGLDSDVADSSILTSGFQPV
SIKPSKADNPHLLTIQPDLSTTTTYQGSLCPRQDGPSPKFQLTNGHLLSPLGGGRHTLHHSSPTSEAE
EFVSRLSTQNYFRSLPRGTSNMTYGTFNFLGGRLMIPNTGISLLIPPDAIPRGKIYEIYLTLHKPEDV
RLPLAGCQTLLSPIVSCGPPGVLLTRPVILAMDHCGEPSPDSWSLRLKKQSCEGSWEQDVLHLGEEAP
SHLYYCQLEASACYVFTEQLGRFALVGEALSVAAAKRLKLLLFAPVACTSLEYNIRVYCLHDTHDALK
EVVQLEKQLGGQLIQEPRVLHFKDSYHNLRLSIHDVPSSLWKSKLLVSYQEIPFYHIWNGTQRYLHCT
FTLERVSPSTSDLACKLWVWQVEGDGQSFSINFNITKDTRFAELLALESEAGVPALVGPSAFKIPFLI
RQKIISSLDPPCRRGADWRTLAQKLHLDSHLSFFASKPSPTAMILNLREARHFPNGNLSQLAAAVAGL
GQPDAGLFTVSEAEC

A ClustalW comparison of the above protein sequences yields the following sequence alignment shown in Table 21B.

TABLE 21 B

Comparison of the NOV21 protein sequences.

| | |
|---|---|
| NOV21a | MAVRPGLWPALLGIVLAAWLRGSGAQQSATVANPVPGANPDLLPHFLVEPEDVYIVKNKP |
| NQV21b | -------------------------QQSATVANPVPGANPDLLPHFLVEPEDVYIVKNKP |
| NOV21c | ----------------------TGSQQSATVANPVPGANPDLLPHFLVEPEDVYIVKNKP |
| NOV21d | -------------------------QQSATVANPVPGANPDLLPHFLVEPEDVYIVKNKP |
| NOV21e | MAVRPGLWPALLGIVLAAWLRGSGAQQSATVANPVPGANPDLLPHFLVEPEDVYIVKNKP |
| NOV21f | -------------------------QQSATVANPVPGANPDLLPHFLVEPEDVYIVKNKP |
| NOV21g | ---------ALLGIVLAAWLRGSGAQQSATVANPVPGANPDLLPHFLVEPEDVYIVKNKP |
| NOV21h | -------------------------QQSATVANPVPGANPDLLPHFLVEPEDVYIVKNKP |
| NOV21a | VLLVCKAVPATQIFFKCNGEWVRQVDHVIERSTDGSSGEPTMEVRINVSRQQVEKVFGLE |
| N0V21b | VLLVCKAVPATQIFFKCNGEWVRQVDHVIERSTDGSSGEPTMEVRINVSRQQVEKVFGLE |
| N0V21c | VLLVCKAVPATQIFFKCNGEWVRQVDHVIERSTDGSSGLPTMEVRINVSRQQVEKVFGLE |
| NOV21d | VLLVCKAVPATQIFFKCNGEWVRQVDHVIERSTDGSSGEPTMEVRINVSRQQVEKVFGLE |
| NOV21e | VLLVCKAVPATQIFFKCNGEWVRQVDHVIERSTDGSSGLPTMEVRINVSRQQVEKVFGLE |
| NOV21f | VLLVCKAVPATQIFFKCNGEWVRQVDHVIERSTDGSSGLPTMEVRINVSRQQVEKVFGLE |
| NOV21g | VLLVCKAVPATQIFFKCNGEWVRQVDHVIERSTDGSSGLPTMEVRINVSRQQVEKVFGLE |
| NOV21h | VLLVCKAVPATQIFFKCNGEWVRQVDHVIERSTDGSSGLPTMEVRINVSRQQVEKVFGLE |
| NOV21a | EYWCQCVAWSSSGTTKSQKAYIRIARLRKNFEQEPLAKEVSLEQGIVLPCRPPEGIPPAE |
| NOV21b | EYWCQCVAWSSSGTTKSQKAYIRIARLRKNFEQEPLAKEVSLEQGIVLPCRPPEGIPPAE |
| NOV21c | EYWCQCVAWSSSGTTKSQKAYIRIAYLRKNFEQEPLAKEVSLEQGIVLPCRPPEGIPPAE |
| NOV21d | EYWCQCVAWSSSGTTKSQKAYIRIARLRKNFEQEPLAKEVSLEQGIVLPCRPPEGIPPAE |
| NOV21e | EYWCQCVAWSSSGTTKSQKAYIRIARLRKNFEQEPLAKEVSLEQGIVLPCRPPEGIPPAE |
| NOV21f | EYWCQCVAWSSSGTTKSQKAYIRIAYLRKNFEQEPLAKEVSLEQGIVLPCRPPEGIPPAE |
| NOV21g | EYWCQCVAWSSSGTTKSQKAYIRIAYLRKNFEQEPLAKEVSLEQGIVLPCRPPEGIPPAE |
| NOV21h | EYWCQCVAWSSSGTTKSQKAYIRIAYLRKNFEQEPLAKEVSLEQGIVLPCRPPEGIPPAE |
| NOV21a | VEWLRNEDLVDPSLDPNVYITREHSLVVRQARLADTANYTCVAXNIVARRRSASAAVIVY |
| NOV21b | VEWLRNEDLVDPSLDPNVYITREHSLVVRQAPLADTANYTCVAKNIVARRRSASAAVIVY |
| NOV21c | VEWLRNEDLVDPSLDPNVYITREHSLVVRQARLADTANYTCVAKNIVARRRSASAAVIVY |
| NOV21d | VEWLRNEDLVDPSLDPNVYITREHSLVVRQARLADTANYTCVAKNIVARRRSASAAVIVY |
| NOV21e | VEWLRNEDLVDPSLDPNVYITREHSLVVRQARLADTANYTCVAKNIVARRRSASAAVIVY |
| NOV21t | VEWLRNEDLVDPSLDPNVYITREHSLVVRQARLADTANYTCVAKNIVARRRSASAAVIVY |
| NOV21g | VEWLRNEDLVDPSLDPNVYITREHSLVVRQARLADTANYTCVAKNIVARRRSASAAVIVY |
| NOV21h | VEWLRNEDLVDPSLDPNVYITREHSLVVRQARLADTANYTCVAKNIVARRRSASAAVIVY |
| NOV21a | VNGGWSTWTEWSVCSASCGRGWQKRSRSCTNPAPLNGGAFCEGQNVHDRTVSSLLVSVDG |
| NOV21b | VNGGWSTWTEWSVCSASCGRGWQKRSRSCTNPAPLNGGAFCEGQNVHDRTVSSLLVSVDG |
| NOV21c | VNGGWSTWTEWSVCSASCGRGWQKRSRSCTNPAPLNGGAFCEGQNVQK-TACATLCPVDG |
| NOV21d | VDG--------------------------------------------------------- |
| NOV21e | VNGGWSTWTEWSVCSASCGRGWQKRSRSCTNPAPLNGGAFCEGQ-NVQKTACATLCPVDG |
| NOV21f | VNGGWSTWTEWSVCSASCGRGWQKRSRSCTNPAPLNGGAFCEGQNVQK-TACATLCPVDG |
| NOV21g | VDG--------------------------------------------------------- |
| NOV21h | VDG--------------------------------------------------------- |
| NOV21a | SWSPWSKWSACGLDCTHWRSRECSDPAPRNGGEECQGTDLDTPNCTSDLCVHSASGPEDV |
| NOV21b | SWSPWSKWSACGLDCTHWRSRECSDPAPRNGGEECQGTDLDTRNCTSDLCVHSASG---- |
| NOV21c | SWSPWSKWSACGLDCTHWRSRECSDPAPRNGGEECQGTDLDTRNCTSDLCVHSASGEFG- |
| NOV21d | SWSPWSKWSACGLDCTHWRSRECSDPAPRNGGEECQGTDLDTRNCTSDLCVHSASGPEDV |
| NOV21e | SWSPWSKWSACGLDCTHWRSRECSDPAPRNGGEECQGTDLDTRNCTSDLCVHSASGPEDV |
| NOV21f | SWSPWSKWSACGLDCTHWRSRECSDPAPRNGGEECQGTDLDTRNCTSDLCVHSASG---- |
| NOV21g | SWSPWSKWSACGLDCTHWRSRECSDPAPRNGGEECQGTDLDTRNCTSDLCVHTASGPEDV |
| NOV21h | SWSPWSKWSACGLGCTHWRSRECSDPAPRNGGEECQGTDLDTRNCTSDLCVHTASGPEDV |
| NOV21a | ALYVGLIAVAVCLVLLLLVLILVYCRKKEGLDSDVADSSILTSGFQPVSIKPSKADNPHL |
| NOV21b | ------------------------------------------------------------ |
| NOV21c | ------------------------------------------------------------ |
| NOV21d | ALYVGLIAVAVCLVLLLLVLILVYCRKKEGLDSDVADSSTLTSGFQPVSIKPSKADN--- |
| NOV21e | ALYVGLIAVAVCLVLLLLVLILVYCRKKEGLDSDVADSSILTSGFQPVSIKPSKADNPHL |
| NOV21f | ------------------------------------------------------------ |
| NOV21g | ALYVGLIAVAVCLVLLLLVLILVYCRKKEGLDSDVADSSILTSGFQPVSIKPSKADNPHL |
| NOV21h | ALYVGLIAVAVCLVLLLLVLILVYCRKKEGLDSDVADSSILTSGFQPVSIKPSKADNPHL |
| NOV21a | LTIQPDLS-TTTTYQGSLCPRQDGPSPKFQLTNGHLLSPLGGGRHTLHHSSPTSEAEEFV |
| NOV21b | ------------------------------------------------------------ |
| NOV21c | ------------------------------------------------------------ |
| NOV21d | ------------------------------------------------------------ |
| NOV21e | LTIQPDLSTTTTTYQGSLCPRQDGPSPKFQLTNGHLLSPLGGGRHTLHHSSPTSEAEEFV |
| NOV21f | ------------------------------------------------------------ |
| NOV21g | LTIQPDLSTTTTTYQGSLCPRQDGPSPKFQLTNGHLLSPLGGGRHTLHHSSPTSEAEEFV |
| NOV21h | LTIQPDLSTTTTTYQGSLCPRQDGPSPKFQLTNGHLLSPLGGGRHTLHHSSPTSEAEEFV |
| NOV21a | SRLSTQNYFRSLPRGTSNNTYGTFNFLGGRLMIPNTGISLLIPPDAIPRGKIYEIYLTLH |
| NOV21b | ------------------------------------------------------------ |
| NOV21c | ------------------------------------------------------------ |
| NOV21d | ------------------------------------------------------------ |
| NOV21e | SRLSTQNYFRSLPRGTSNNTYGTFNFLGGRLMIPNTGISLLIPPDAIPRGKIYEIYLTLH |
| NOV21f | ------------------------------------------------------------ |
| NOV21g | SRLSTQNYFRSLPRGTSNMTYGTFNFLGGRLMIPNTGISLLIPPDAIPRGKIYEIYLTLH |
| NOV21h | SRLSTQNYFRSLPRGTSNMTYGTFNFLGGRLMIPNTGISLLIPPDAIPRGKIYEIYLTLH |
| NOV21a | KPEDVRLPLAGCQTLLSPIVSCGPPGVLLTRPVILAMDHCGEPSPDSWSLRLKKQSCEGS |
| NOV21b | ------------------------------------------------------------ |
| NOV21c | ------------------------------------------------------------ |
| NOV21d | ------------------------------------------------------------ |
| NOV21e | KFEDVRLPLAGCQTLLSPIVSCGPPGVLLTRPVILAMDHCGEPSPDSWSLRLKKQSCEGS |
| NOV21f | ------------------------------------------------------------ |
| NOV21g | KPEDVRLPLAGCQTLLSPIVSCGPPGVLLTRPVILAMDHCGEPSPDSWSLRLKKQSCEGS |
| NOV21h | KPEDVRLPLAGCQTLLSPIVSCGPPGVLLTRPVILAMDHCGEPSPDSWSLRLKKQSCEGS |
| NOV21a | WEQDVLHLGEEAPSHLYYCQLEASACYVFTEQLGRFALVGEALSVAAAKRLKLLLFAPVA |
| NOV21b | ------------------------------------------------------------ |
| NOV21c | ------------------------------------------------------------ |
| NOV21d | ------------------------------------------------------------ |
| NOV21e | WED-VLHLGEEAPSHLYYCQLEASACYVFTEQLGRFALVGEALSVAAAKRLKLLLFAPVA |
| NOV21f | ------------------------------------------------------------ |
| NOV21g | WED-VLHLGEEAPSHLYYCQLEASACYVFTEQLGRFALVGEALSVAAAKRLKLLLFAPVA |
| NOV21h | WED-VLHLGEEAPSHLYYCQLEASACYVFTEQLGRFALVGEALSVAAAKRLKLLLFAPVA |
| NOV21a | CTSLEYNIRVYCLHDTHDALKEVVQLEKQLGGQLIQEPRVLHFKDSYHNLRLSIHDVPSS |
| NOV21b | ------------------------------------------------------------ |
| NOV21c | ------------------------------------------------------------ |
| NOV21d | ------------------------------------------------------------ |
| NOV21e | CTSLEYNIRVYCLHDTHDALKEVVQLEKQLGGQLIQEPRVLHFKDSYHNLRLSIHDVPSS |
| NOV21f | ------------------------------------------------------------ |
| NOV21g | CTSLEYNIRVYCLHDTHDALKEVVQLEKQLGGQLIQEPRVLHFKDSYHNLRLSIHDVPSS |
| NOV21h | CTSLEYNIRVYCLHDTHDALKEVVQLEKQLGGQLIQEPRVLHFKDSYHNLRLSIHDVPSS |
| NOV21a | LWKSKLLVSYQEIPFYHIWNGTQRYLHCTFTLERVSPSTSDLACKLWVWQVEGDGQSFSI |
| NOV21b | ------------------------------------------------------------ |
| NOV21c | ------------------------------------------------------------ |
| NOV21d | ------------------------------------------------------------ |
| NOV21e | LWKSKLLVSYQEIPFYHIWNGTQRYLHCTFTLERVSPSTSDLACKLWVWQVEGDGQSFSI |
| NOv21f | ------------------------------------------------------------ |
| NOV21g | LWKSKLLVSYQEIPFYHIWNGTQRYLHCTFTLERVSPSTSDLACKLWVWQVEGDGQSFSI |
| NOV21h | LWKSKLLVSYQEIPFYHIWNGTQRYLHCTFTLERVSPSTSDLACKLWVWQVEGDGQSFSI |
| NOV21a | NFNITKDTRFAELLALESEAGVPALVGPSAFKIPFLIRQKIISSLDPPCRRGADWRTLAQ |
| NOV21b | ------------------------------------------------------------ |
| NOV21c | ------------------------------------------------------------ |
| NOV21d | ------------------------------------------------------------ |
| NOV21e | NFNITKDTRFAELLALESEAGVPALVGPSAFKIPFLIRQKIISSLDPPCRRGADWRTLAQ |
| NOV21f | ------------------------------------------------------------ |
| NOV21g | NFNITKDTRFAELLALESEAGVPALVGPSAFKIPFLIRQKIISSLDPPCRRGADWRTLAQ |
| NOV21h | NFNITKDTRFAELLALESEAGVPALVGPSAFKIPFLIRQKIISSLDPPCRRGADWRTLAQ |
| NOV21a | KLHLDSHLSFFASKPSPTAMILNLWEARHFPNGNLSQLAAAVAGLGQPDAGLFTVSEAEC |
| NOV21b | ------------------------------------------------------------ |
| NOV21c | ------------------------------------------------------------ |
| NOV21d | ------------------------------------------------------------ |
| NOV21e | KLHLDSHLSFFASKPSPTAMILNLWEARHFPNGNLSQLAAAVAGLGQPDAGLFTVSEAEC |
| NOV21f | ------------------------------------------------------------ |
| NOV21g | KLHLDSHLSFFASKPSPTAMILNLWEARHFPNGNLSQLAAAvAGLGQPDAGLFTVSEAEC |
| NOV21h | KLHLDSHLSFFASKPSPTAMILNLWEARHFPNGNLSQLAAAVAGLGQPDAGLFTVSEAEC |
| NOV21a | (SEQ ID NO: 506) |
| NOV21b | (SEQ ID NO: 508) |
| NOV21c | (SEQ ID NO: 510) |
| NOV21d | (SEQ ID NO: 512) |
| NOV21e | (SEQ ID NO: 514) |
| NOV21f | (SEQ ID NO: 516) |
| NOV21g | (SEQ ID NO: 518) |
| NOV21h | (SEQ ID NO: 520) |

Further analysis of the NOV21a protein yielded the following properties shown in Table 21C.

TABLE 21C
Protein Sequence Properties NOV21a

| | |
|---|---|
| SignalP analysis: | Cleavage site between residues 26 and 27 |
| PSORT II analysis: | |

| PSG: | a new signal peptide prediction method | |
|---|---|---|
| | N-region: | length 4; pos. chg 1; neg. chg 0 |
| | H-region: | length 16; peak value 9.57 |
| | PSG score: | 5.17 |

| | |
|---|---|
| GvH: | von Heijne's method for signal seq. recognition |
| | GvH score (threshold: −2.1): 1.79 |
| | possible cleavage site: between 25 and 26 |

>>> Seems to have a cleavable signal peptide (1 to 25)

| ALOM: | Klein et al's method for TM region allocation | | |
|---|---|---|---|
| | Init position for calculation: 26 | | |
| | Tentative number of TMS(s) for the threshold 0.5: 1 | | |
| | Number of TMS(s) for threshold 0.5: 1 | | |
| | INTEGRAL | Likelihood = | Transmembrane 367-383 |
| | | −19.00 | |

| | |
|---|---|
| PERIPHERAL | Likelihood = 0.85 (at 61) |

| | |
|---|---|
| | ALOM score: −19.00 (number of TMSs: 1) |
| MTOP: | Prediction of membrane topology (Hartmann et al.) |
| | Center position for calculation: 12 |
| | Charge difference: −1.0 C(1.0)-N(2.0) |
| | N >= C: N-terminal side will be inside |

>>> membrane topology: type 1a (cytoplasmic tail 384 to 899)

| MITDISC: | discrimination of mitochondrial targeting seq | | | |
|---|---|---|---|---|
| | R content: | 2 | Hyd Moment(75): | 9.35 |
| | Hyd Moment(95): | 6.17 | G content: | 5 |
| | D/E content: | 1 | S/T content: | 3 |

| | |
|---|---|
| | Score: −4.19 |
| Gavel: | prediction of cleavage sites for mitochondrial preseq |
| | R-2 motif at 31 LRG\|SG |

NUCDISC: discrimination of nuclear localization signals

| | |
|---|---|
| | pat4: none |
| | pat7: none |
| | bipartite: none |
| | content of basic residues: 9.2% |
| | NLS Score: −0.47 |
| KDEL: | ER retention motif in the C-terminus: none |

ER Membrane Retention Signals:

XXRR-like motif in the N-terminus: AVRP

none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: found

LL at 420
LL at 455
LL at 519
LL at 554
LL at 567
LL at 652
LL at 653
LL at 725

checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 70.6 |
| COIL: | Lupas's algorithm to detect coiled-coil regions |
| | total: 0 residues |

Final Results (k = 9/23):

44.4%: endoplasmic reticulum
22.2%: Golgi
11.1%: plasma membrane
11.1%: vesicles of secretory system
11.1%: extracellular, including cell wall

>> prediction for CG53908-01 is end (k = 9)

A search of the NOV21 a protein against the Geneseq database, a proprietary database that contains sequences published in patents and patent publication, yielded several homologous proteins shown in Table 21D.

TABLE 21D
Geneseq Results for NOV21a

| | | NOV21a | Identities/ | | |
|---|---|---|---|---|---|
| | | Residues/ | Similarities for | | |
| Geneseq | Protein/Organism/Length | Match | the Matched | | Expect |
| Identifier | [Patent #, Date] | Residues | Region | | Value |
| AAU79939 | Human UNC5-like protein NOV1 - | 1 . . . 899 | 899/899 | (100%) | 0.0 |
| | Homo sapiens, 899 aa. | 1 . . . 899 | 899/899 | (100%) | |
| | [WO200229038-A2, 11-APR-2002] | | | | |
| AAU85403 | Human protein NOV1 - Homo sapiens, | 1 . . . 899 | 888/900 | (98%) | 0.0 |
| | 898 aa. [WO200210216-A2, 07-FEB- | 1 . . . 898 | 890/900 | (98%) | |
| | 2002] | | | | |
| AAU97899 | Human netrin binding membrane | 1 . . . 899 | 886/900 | (98%) | 0.0 |
| | receptor UNC5H-1 protein - Homo | 1 . . . 898 | 889/900 | (98%) | |
| | sapiens, 898 aa. [WO200233080-A2, | | | | |
| | 25-APR-2002] | | | | |
| AAU10543 | Rat netrin receptor UNC5H1 (YSG7) | 1 . . . 899 | 852/900 | (94%) | 0.0 |
| | polypeptide - Rattus sp, 898 aa. | 1 . . . 898 | 871/900 | (96%) | |
| | [WO200175440-A2, 11-OCT-2001] | | | | |
| AAU97900 | Rat netrin binding membrane receptor | 1 . . . 899 | 852/900 | (94%) | 0.0 |
| | UNC5H-1 protein - Rattus sp, 898 aa. | 1 . . . 898 | 871/900 | (96%) | |
| | [WO200233080-A2, 25-APR-2002] | | | | |

In a BLAST search of public sequence databases, the NOV21a protein was found to have homology to the proteins shown in the BLASTP data in Table 21E.

TABLE 21E
Public BLASTP Results for NOV21a

| | | NOV21a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8K1S4 | Netrin receptor Unc5h1 - Mus | 1 . . . 899 | 858/900 (95%) | 0.0 |
| | musculus (Mouse), 898 aa. | 1 . . . 898 | 879/900 (97%) | |
| O08721 | Transmembrane receptor UNC5H1 - | 1 . . . 899 | 852/900 (94%) | 0.0 |
| | Rattus norvegicus (Rat), 898 aa. | 1 . . . 898 | 871/900 (96%) | |
| Q96GP4 | Similar to transmembrane receptor | 359 . . . 899 | 540/542 (99%) | 0.0 |
| | Unc5H1 - Homo sapiens (Human), | 4 . . . 544 | 540/542 (99%) | |
| | 544 aa (fragment). | | | |
| O08747 | Rostral cerebellar malformation | 9 . . . 898 | 515/913 (56%) | 0.0 |
| | protein - Mus musculus (Mouse), 931 | 26 . . . 930 | 668/913 (72%) | |
| | aa. | | | |
| Q8CD16 | Unc5 homolog - Mus musculus | 9 . . . 898 | 515/932 (55%) | 0.0 |
| | (Mouse), 950 aa. | 26 . . . 949 | 668/932 (71%) | |

PFam analysis predicts that the NOV21a protein contains the domains shown in the Table 21F.

TABLE 21F
Domain Analysis of NOV21a

| | | Identities/ | | |
|---|---|---|---|---|
| | | Similarities | | |
| Pfam | NOV21a | for the | | Expect |
| Domain | Match Region | Matched Region | | Value |
| ig | 163 . . . 223 | 17/63 | (27%) | 0.0015 |
| | | 38/63 | (60%) | |
| tsp_1 | 246 . . . 287 | 20/53 | (38%) | 0.00036 |
| | | 30/53 | (57%) | |
| tsp_1 | 303 . . . 350 | 21/53 | (40%) | 0.0017 |
| | | 32/53 | (60%) | |
| ZU5 | 495 . . . 598 | 47/115 | (41%) | 6.3e−56 |
| | | 103/115 | (90%) | |
| death | 818 . . . 898 | 26/88 | (30%) | 3.5e−14 |
| | | 64/88 | (73%) | |

### Example 22.

The NOV22 clone was analyzed, and the nucleotide and encoded polypeptide sequences are shown in Table 22A.

TABLE 22A
NOV22 Sequence Analysis

| | | |
|---|---|---|
| NOV22a, CG53944-02 | SEQ ID NO: 547 | 1887 bp |
| DNA Sequence | ORF Start: ATG at 1 | ORF Stop: TAG at 1885 |

ATGGCCATCCTCCCGTTGCTCCTGTGCCTGCTGCCGCTGGCCCCTGCCTCATCCCCACCCCAGTCAGC
CACACCCAGCCCATGTCCCCGCCGCTGCCGCTGCCAGACACAGTCGCTGCCCCTAAGCGTGCTGTGCC
CAGGGGCAGGCCTCCTGTTCGTGCCACCCTCGCTGGACCGCCGGGCAGCCGAGCTGCGGCTGGCAGAC
AACTTCATCGCCTCCGTGCGCCGCCGCGACCTGGCCAACATGACAGGCCTGCTGCATCTGAGCCTGTC
GCGGAACACCATCCGCCACGTGGCTGCCGGCGCCTTCGCCGACCTGCGGGCCCTGCGTGCCCTGCACC
ATCCTCAGCAACAACCAGCTGGCAGCGCTGGCGGCCGGCGCCCTGGATGATTGTGCCGAGACACTGGA
GGACCTCGACCTCTCCTACAACAACCTCGAGCAGCTGCCCTGGGAGGCCCTGGGCCGCCTGGGCAACG
TCAACACGTTGGGCCTCGACCACAACCTGCTGGCTTCTGTGCCCGCCGGCGCTTTTTCCCGCCTGCAC
AAGCTGGCCCGGCTGGACATGACCTCCAACCGCCTGACCACAATCCCACCCGACCCACTCTTCTCCCG
CCTGCCCCTGCTCGCCAGGCCCCGGGGCTCGCCCGCCTCTGCCCTGGTGCTGGCCTTTGGCGGGAACC
CCCTGCACTGCAACTGCGAGCTGGTGTGGCTGCGTCGCCTGGCGCGGGAGGACGACCTCGAGGCCTGC
GCGTCCCCACCTGCTCTGGGCGGCCGCTACTTCTGGGCGGTGGGCGAGGAGGAGTTTGTCTGCGAGCC
GCCCGTGGTGACTCACCGCTCACCACCTCTGGCTGTGCCCGCAGGTCGGCCGGCTGCCCTGCGCTGCC
GGGCAGTGGGGGACCCAGAGCCCCGTGTGCGTTGGGTGTCACCCCAGGGCCGGCTGCTAGGCAACTCA
AGCCGTGCCCGCGCCTTCCCCAATGGGACGCTGGAGCTGCTGGTCACCGAGCCGGGTGATGGTGGCAT
CTTCACCTGCATTGCGGCCAATGCAGCTGGCGAGGCCACAGCTGCTGTGGAGCTGACTGTGGGTCCCC
CACCACCTCCTCAGCTAGCCAACAGCACCAGCTGTGACCCCCCGCGGGACGGGGATCCTGATGCTCTC
ACCCCACCCTCCGCTGCCTCTGCTTCTGCCAAGGTGGCCGACACTGGGCCCCCTACCGACCGTGGCGT
CCAGGTGACTGAGCACGCGGCCACAGCTGCTCTTGTCCAGTGGCCGGATCAGCGGCCTATCCCGGGCA
GAGAGCCGCTCGTTCCTGCTGACGGACCTGGCGTCAGGCCGGACCTACGATCTGTGCGTGCTCGCCGT
GTATGAGGACAGCGCCACGGGGCTCACGGCCACGCGGCCTGTGGGCTGCGCCCGCTTCTCCACCGAAC
CTGCGCTGCGGCCATGCGGGGCGCCGCACGCTCCCTTCCTGGGCGGCACGATGATCATCGCGCTGGGC
GGCGTCATCGTAGCCTCGGTACTGGTCTTCATCTTCGTGCTGCTAATGCGCTACAAGGTGCACGGCGG
CCAGCCCCCCGGCAAGGCCAAGATTCCCGCGCCTGTTAGCAGCGTTTGCTCCCAGACCAACGGCGCCC
TGGGCCCCACGCCCACGCCCGCCCCGCCCGCCCCGGAGCCCGCGGCGCTCAGGGCCCACACCGTGGTC
CAGCTGGACTGCGAGCCCTGGGGGCCCGGCCACGAACCTGTGGGACCCTAG

| | | | |
|---|---|---|---|
| NOV22a, CG53944-02 | | | |
| Protein Sequence | SEQ ID NO: 548 | 1628 aa | MW at 66259.1kD |

MAILPLLLCLLPLAPASSPPQSATPSPCPPRCRCQTQSLPLSVLCPGAGLLFVPPSLDRRAAELRLAD
NFIASVRRRDLANMTGLLHLSLSRNTIRHVAAGAFADLPALRALHLDGNRLTSLGEGQLRGLVNLRHL
ILSNNQLAALAAGALDDCAETLEDLDLSYNNLEQLPWEALGRLGNVNTLGLDHNLLASVPAGAFSRLH
KLARLDMTSNRLTTIPPDPLFSRLPLLARPRGSPASALVLAFGGNPLHCNCELVWLRRLAREDDLEAC
ASPPALGGRYFWAVGEEEFVCEPPVVTHRSPPLAVPAGRPAALRCRAVGDPEPRVRWVSPQGRLLGNS
SRAPAFPNGTLELLVTEPGDGGIFTCIAANAAGEATAAVELTVGPPPPPQLANSTSCDPPRDGDPDAL
TPPSAASASAKVADTGPPTDRGVQVTEHGATAALVQWPDQRPIPGIRMYQIQYNSSADDILVYRMIPA
ESRSFLLTDLASGRTYDLCVLAVYEDSATGLTATRPVGCARFSTEPALRPCGAPHAPFLGGTMIIALG
GVIVASVLVFIFVLLMRYKVHGGQPPGKAKIPAPVSSVCSQTNGALGPTPTPAPPAPEPAALRAHTVV
QLDCEPWGPGHEPVGP

| | | |
|---|---|---|
| NOV22b, CG53944-01 | SEQ ID NO: 549 | 1887 bp |
| DNA Sequence | ORF Start: ATG at 1 | ORF Stop: TAG at 1885 |

ATGGCCATCCTCCCGTTGCTCCTGTGCCTGCTGCCGCTGGCCCCTGCCTCATCCCCACCCCAGTCAGC
CACACCCAGCCCATGTCCCCGCCGCTGCCGCTGCCAGACACAGTCGCTGCCCCTAAGCGTGCTGTGCC
AACTTCATCGCCTCCGTGCGCCGCCGCGACCTGGCCAACATGACAGGCCTGCTGCATCTGAGCCTGTC
GCGGAACACCATCCGCCACGTGGCTGCCGGCGCCTTCGCCGACCTGCGGGCCCTGCGTGCCCTGCACC
TGGATGGCAACCGGCTGACCTCACTGGGCGAGGGCCAGCTGCGCGGCCTGGTCAACTTGCGCCACCTC
ATCCTCAGCAACAACCAGCTGGCAGCGCTGGCGGCCGGCGCCCTGGATGATTGTGCCGAGACACTGGA
GGACCTCGACCTCTCCTACAACAACCTCGAGCAGCTGCCCTGGGAGGCCCTGGGCCGCCTGGGCAACG
TCAACACGTTGGGCCTCGACCACAACCTGCTGGCTTCTGTGCCCGCCGGCGCTTTTTCCCGCCTGCAC
AAGCTGGCCCGGCTGGACATGACCTCCAACCGCCTGACCACAATCCCACCCGACCCACTCTTCTCCCG
CCTGCCCCTGCTCGCCAGGCCCCGGGGCTCGCCCGCCTCTGCCCTGGTGCTGGCCTTTGGCGGGAACC
CCCTGCACTGCAACTGCGAGCTGGTGTGGCTGCGTCGCCTGGCGCGGGAGGACGACCTCGAGGCCTGC
GCGTCCCCACCTGCTCTGGGCGGCCGCTACTTCTGGGCGGTGGGCGAGGAGGAGTTTGTCTGCGAGCC
GCCCGTGGTGACTCACCGCTCACCACCTCTGGCTGTGCCCGCAGGTCGGCCGGCTGCCCTGCGCTGCC
GGGCAGTGGGGGACCCAGAGCCCCGTGTGCGTTGGGTGTCACCCCAGGGCCGGCTGCTAGGCAACTCA
AGCCGTGCCCGCGCCTTCCCCAATGGGACGCTGGAGCTGCTGGTCACCGAGCCGGGTGATGGTGGCAT
CTTCACCTGCATTGCGGCCAATGCAGCTGGCGAGGCCACAGCTGCTGTGGAGCTGACTGTGGGTCCCC
CACCACCTCCTCAGCTAGCCAACAGCACCAGCTGTGACCCCCCGCGGGACGGGGATCCTGATGCTCTC
ACCCCACCCTCCGCTGCCTCTGCTTCTGCCAAGGTGGCCGACACTGGGCCCCCTACCGACCGTGGCGT
CCAGGTGACTGAGCACGGGGCCACAGCTGCTCTTGTCCAGTGGCCGGATCAGCGGCCTATCCCGGGCA
TCCGCATGTACCAGATCCAGTACAACAGCTCGGCTGATGACATCCTCGTCTACAGGATGATCCCGGCG
GAGAGCCGCTCGTTCCTGCTGACGGACCTGGCGTCAGGCCGGACCTACGATCTGTGCGTGCTCGCCGT
GTATGAGGACAGCGCCACGGGGCTCACGGCCACGCGGCCTGTGGGCTGCGCCCGCTTCTCCACCGAAC
CTGCGCTGCGGCCATGCGGGGCGCCGCACGCTCCCTTCCTGGGCGGCACGATGATCATCGCGCTGGGC
GGCGTCATCGTAGCCTCGGTACTGGTCTTCATCTTCGTGCTGCTAATGCGCTACAAGGTGCACGGCGG
CCAGCCCCCCGGCAAGGCCAAGATTCCCGCGCCTGTTAGCAGCGTTTGCTCCCAGACCAACGGCGCCC
TGGGCCCCACGCCCACGCCCGCCCCGCCCGCCCCGGAGCCCGCGGCGCTCAGGGCCCACACCGTGGTC
CAGCTGGACTGCGAGCCCTGGGGGCCCGGCCACGAACCTGTGGGACCCTAG

| | | | |
|---|---|---|---|
| NOV22b, CG53944-01 | | | |
| Protein Sequence | SEQ ID NO: 550 | 628 aa | MW at 66259.1kD |

MAILPLLLCLLPLAPASSPPQSATPSPCPRRCRCQTQSLPLSVLCPGAGLLFVPPSLDRRAAELRLAD
NFIASVRRRDLANMTGLLHLSLSRNTIRHVAAGAFADLRALRALHLDGNRLTSLGEGQLRGLVNLRHL
ILSNNQLAALAAGALDDCAETLEDLDLSYNNLEQLPWEALGRLGNVNTLGLDHNLLASVPAGAFSRLH
KLARLDMTSNRLTTIPPDPLFSRLPLLARPRGSPASALVLAFGGNPLHCNCELVWLRRLAREDDLEAC
ASPPALGGRYFWAVGEEEFVCEPPVVTHRSPPLAVPAGRPAALRCRAVGDPEPRVRWVSPQGRLLGNS
SRAPAFPNGTLELLVTEPGDGGIFTCIAANAAGEATAAVELTVGPPPPPQLANSTSCDPPRDGDPDAL
TPPSAASASAKVADTGPPTDRGVQVTEHGATAAIJVQWPDQRPIPGIRMYQIQYNSSADDILVYRMPA
ESRSFLLTDLASGRTYDLCVLAVYEDSATGLTATRPVGCARFSTEPALRPCGAPHAPFLGGTMIIALG
GVIVASVLVFIFVLLMRYKVHGGQPPGKAKIPAPVSSVCSQTNGALGPTPTPAPPAPEPAALRAHTVV
QLDCEPWGPGHEPVGP

A ClustalW comparison of the above protein sequences yields the following sequence alignment shown in Table 22B.

TABLE 22B

Comparison of the NOV22 protein sequences.

| | |
|---|---|
| NOV22a | MAILPLLLCLLPLAPASSPPQSATPSPCPRRCRCQTQSLPLSVLCPGAGLLFVPPSLDRR |
| NOV22b | MAILPLLLCLLPLAPASSPPQSATPSPCPRRCRCQTQSLPLSVLCPGAGLLFVPPSLDRR |
| NOV22a | AAELRLADNFIASVRPRDLANMTGLLHLSLSRNTIRHVAAGAFADLEALRALHLDGNRLT |
| NOV22b | AAELRLADNFIASVRRRDLANMTGLLHLSLSRNTIRHVAAGAFADLRALRALHLDGNRLT |
| NOV22a | SLGEGQLRGLVNLRHLILSNNQLAALAAGALDDCAETLEDLDLSYNNLEQLPWEALGRLG |
| NOV22b | SLGEGQLRGLVNLRHLILSNNQLAALAAGALDDCAETLEDLDLSYNNLEQLPWEALGRLG |
| NOV22a | NVNTLGLDHNLLASVPAGAFSRLHKLARLDMTSNRLTTIPPDPLFSRLPLLARPRGSPAS |
| NOV22b | NVNTLGLDHNLLASVPAGAFSRLHKLARLDMTSNRLTTIPPDPLFSRLPLLARPRGSPAS |
| NOV22a | ALVLAFGGNPLHCNCELVWLRRLAREDDLEACASPPALGGRYFWAVGEEEFVCEPPVVTH |
| NOV22b | ALVLAFGGNPLHCNCELVWLRRLAREDDLEACASPPALGGRYFWAVGEEEFVCEPPVVTH |
| NOV22a | RSPPLAVPAGRPAALRCRAVGDPEPRVRWVSPQGRLLGNSSRARAFPNGTLELLVTEPGD |
| NOV22b | RSPPLAVPAGRPAALRCRAVGDPEPRVRWVSPQGRLLGNSSRARAFPNGTLELLVTEPGD |
| NOV22a | GGIFTCIAANAAGEATAAVELTVGPPPPPQLANSTSCDPPRDGDPDALTPPSAASASAKV |
| NOV22b | GGIFTCIAANAAGEATAAVELTVGPPPPPQLANSTSCDPPRDGDPDALTPPSAASASAKV |
| NOV22a | ADTGPPTDRGVQVTEHGATAALVQWPDQRPIPGIRMYQIQYNSSADDILVYRMIPAESRS |
| NOV22b | ADTGPPTDRGVQVTEHGATAALVQWPDQRPIPGIRMYQIQYNSSADDILVYRMIPAESRS |
| NOV22a | FLLTDLASGRTYDLCVLAVYEDSATGLTATRPVGCARFSTEPALRPCGAPHAPFLGGTMI |
| NOV22b | FLLTDLASGRTYDLCVLAVYEDSATGLTATRPVGCARFSTEPALRPCGAPHAPFLGGTMI |
| NOV22a | IALGGVIVASVLVFIFVLLMRYKVHGGQPPGKAKIPAPVSSVCSQTNGALGPTPTPAPPA |
| NOV22b | IALGGVIVASVLVFIFVLLMRYKVHGGQPPGKAKIPAPVSSVCSQTNGALGPTPTPAPPA |
| NOV22a | PEPAALRAHTVVQLDCEPWGPGHEPVGP |
| NOV22b | PEPAALRAHTVVQLDCEPWGPGHEPVGP |
| NOV22a | (SEQ ID NO: 548) |
| NOV22b | (SEQ ID NO: 550) |

Further analysis of the NOV22a protein yielded the following properties shown in Table 22C.

TABLE 22C
Protein Sequence Properties NOV22a

| | |
|---|---|
| SignalP analysis: | Cleavage site between residues 17 and 18 |
| PSORT II analysis: | |

| PSG: | a new signal peptide prediction method | |
|---|---|---|
| | N-region: | length 0; pos. chg 0; neg. chg 0 |
| | H-region: | length 29; peak value 9.99 |
| | PSG score: | 5.59 |

| | |
|---|---|
| GvH: | von Heijne's method for signal seq. recognition |
| | GvH score (threshold: −2.1): 4.65 |
| | possible cleavage site: between 16 and 17 |

>>> Seems to have a cleavable signal peptide (1 to 16)

| ALOM: | Klein et al's method for TM region allocation | | |
|---|---|---|---|
| | Init position for calculation: 17 | | |
| | Tentative number of TMS(s) for the threshold 0.5: 2 | | |
| | Number of TMS(s) for threshold 0.5: 1 | | |
| | INTEGRAL | Likelihood = | Transmembrane 543-559 |
| | | −12.79 | |

| | |
|---|---|
| PERIPHERAL | Likelihood = 2.44 (at 363) |

| | |
|---|---|
| | ALOM score: −12.79 (number of TMSs: 1) |
| MTOP: | Prediction of membrane topology (Hartmann et al.) |
| | Center position for calculation: 8 |
| | Charge difference: 2.0 C(3.0)-N(1.0) |
| | C > N: C-terminal side will be inside |

>>>Caution: Inconsistent mtop result with signal peptide
>>> membrane topology: type 1a (cytoplasmic tail 560 to 628)

| MITDISC: | discrimination of mitochondrial targeting seq | | | |
|---|---|---|---|---|
| | R content: | 3 | Hyd Moment (75): | 2.94 |
| | Hyd Moment (95): | 1.64 | G content: | 2 |
| | D/E content: | 1 | S/T content: | 9 |

| | |
|---|---|
| | Score: −1.46 |
| Gavel: | prediction of cleavage sites for mitochondrial preseq |
| | R-2 motif at 43 CRC\|QT |

NUCDISC: discrimination of nuclear localization signals

| | |
|---|---|
| | pat 4: none |
| | pat7: PCPRRCR (3) at 27 |
| | pat7: PRRCRCQ (5) at 29 |
| | bipartite: none |
| | content of basic residues: 8.4% |
| | NLS Score: 0.22 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 55.5 |
| COIL: | Lupas's algorithm to detect coiled-coil regions |
| | total: 0 residues |

Final Results (k = 9/23):

44.4%: extracellular, including cell wall
22.2%: Golgi
22.2%: endoplasmic reticulum
11.1%: plasma membrane

>> prediction for CG53944-02 is exc (k = 9)

A search of the NOV22a protein against the Geneseq database, a proprietary database that contains sequences published in patents and patent publication, yielded several homologous proteins shown in Table 22D.

TABLE 22D
Geneseq Results for NOV22a

| | | NOV22a | Identities/ | | |
|---|---|---|---|---|---|
| | | Residues/ | Similarities for | | |
| Geneseq | Protein/Organism/Length | Match | the Matched | | Expect |
| Identifier | [Patent #, Date] | Residues | Region | | Value |
| ABP69326 | Human polypeplide SEQ ID NO 1373 - | 1 . . . 628 | 628/628 | (100%) | 0.0 |
| | Homo sapiens, 628 aa. | 1 . . . 628 | 628/628 | (100%) | |
| | [WO200270539-A2, 12-SEP-2002] | | | | |
| AAG67523 | Amino acid sequence of a human | 1 . . . 628 | 628/628 | (100%) | 0.0 |
| | secreted polypeptide - Homo sapiens, | 1 . . . 628 | 628/628 | (100%) | |
| | 628 aa. [WO200166690-A2, 13-SEP-2001] | | | | |
| AAB84469 | Amino acid sequence of an interferon | 1 . . . 628 | 628/628 | (100%) | 0.0 |
| | omega-1 like protein NOV2 - Homo | 1 . . . 628 | 628/628 | (100%) | |
| | sapiens, 628 aa. [WO200142471-A2, | | | | |
| | 14-JUN-2001] | | | | |
| AAG65805 | Human leucine-rich repeat (LRR) family | 1 . . . 628 | 626/628 | (99%) | 0.0 |
| | member, 33395 polypeptide - Homo | 1 . . . 628 | 627/628 | (99%) | |
| | sapiens, 628 aa. [WO200172827-A2, | | | | |
| | 04-OCT-2001] | | | | |
| ABG34079 | Human Pro peptide #51 - Homo | 1 . . . 628 | 627/628 | (99%) | 0.0 |
| | sapiens, 627 aa. [WO200224888-A2, | 1 . . . 627 | 627/628 | (99%) | |
| | 28-MAR-2002] | | | | |

In a BLAST search of public sequence databases, the NOV22a protein was found to have homology to the proteins shown in the BLASTP data in Table 22E.

TABLE 22E
Public BLASTP Results for NOV22a

| | | NOV22a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| Q9BTNO | Hypothetical protein - Homo sapiens | 1 . . . 628 | 628/628 | (100%) | 0.0 |
| | (Human), 628 aa. | 1 . . . 628 | 628/628 | (100%) | |
| CAD10239 | Sequence 1 from Patent WO0172827 - | 1 . . . 628 | 626/628 | (99%) | 0.0 |
| | Homo sapiens (Human), 628 aa. | 1 . . . 628 | 627/628 | (99%) | |
| Q8BLY3 | Hypothetical leucine-rich repeat | 1 . . . 628 | 603/628 | (96%) | 0.0 |
| | containing protein - Mus musculus | 1 . . . 626 | 611/628 | (97%) | |
| | (Mouse), 626 aa. | | | | |
| Q9ULH4 | Hypothetical protein KIAA1246 - | 23 . . . 603 | 310/585 | (52%) | e−174 |
| | Homo sapiens (Human), 832 aa | 59 . . . 639 | 398/585 | (67%) | |
| | (fragment). | | | | |
| Q9BE71 | Hypothetical 84.7 kDa protein - | 23 . . . 603 | 309/585 | (52%) | e−174 |
| | Macaca fascicularis (Crab eating | 16 . . . 596 | 397/585 | (67%) | |
| | macaque) (Cynomolgus monkey), | | | | |
| | 789 aa. | | | | |

PFam analysis predicts that the NOV22a protein contains the domains shown in the Table 22F.

TABLE 22F
Domain Analysis of NOV22a

| | | Identities/ | |
|---|---|---|---|
| | | Similarities | |
| Pfam | NOV22a Match | for the Matched | |
| Domain | Region | Region | Expect Value |
| LRR | 84 . . . 107 | 8/25 (32%) | 0.86 |
| | | 21/25 (84%) | |
| LRR | 132 . . . 155 | 10/25 (40%) | 0.27 |
| | | 20/25 (80%) | |
| LRR | 157 . . . 180 | 12/25 (48%) | 0.026 |
| | | 19/25 (76%) | |
| LRRCT | 249 . . . 294 | 21/55 (38%) | 4.3e−05 |
| | | 37/55 (67%) | |
| ig | 310 . . . 368 | 15/62 (24%) | 1.2e−05 |
| | | 44/62 (71%) | |
| fn3 | 425 . . . 505 | 18/84 (21%) | 7.1e−05 |
| | | 55/84 (65%) | |

### Example 23.

The NOV23 clone was analyzed, and the nucleotide and encoded polypeptide sequences are shown in Table 23A.

TABLE 23A

NOV23 Sequence Analysis

| | | |
|---|---|---|
| NOV23a, CG54308-04 | SEQ ID NO: 551 | 1284 bp |
| DNA Sequence | ORF Start: ATG at 1 | ORF Stop: TAA at 1276 |

ATGGACTCTCTTGTTACAGCAAACACCAAATTTTGCTTTGATCTTTTTCAAGAGATAGGCAAAGATGA
TCGTCATAAAAACATATTTTTCTCTCCCCTGAGCCTCTCAGCTGCCCTTGGTATGGTACGCTTGGGTG
CTAGAAGTGACAGTGCACATCAGATTGATGAGGTACTACACTTCGACGAATTTTCCCAGAATGAAAGC
AAAGAACCTGACCCTTGTCTGAAAAGCAACAAACAAAAGTGCTGGCTGACAGCTCTCTGGAGGGGGCA
GAAAAAAACGACAGAGCCTCTGGATCAGCAGGCTGGGTCCTTAAACAATGAGAGCGGACTGGTCAGCT
GCTACTTTGGGCAGCTTCTCTCCAAATTAGACAGGATCAAGACTGATTACACACTGAGTATTGCCAAC
AGGCTTTATGGAGAGCAGGAATTCCCAATCTGTCAGGAATACTTAGATGGTGTGATTCAATTTTACCA
CACGACGATTGAAAGTGTTGATTTCCAAAAAAACCCTGAAAAATCCAGACAAGAGATTAACTTCTGGG
TTGAATGTCAATCCCAAGGTAAAATCAAGGAACTCTTCAGCAAGGACGCTATTAATGCTGAGACTGTG
CTGGTACTGGTGAATGCTGTTTACTTCAAGGCCAAATGGGAAACATACTTTGACCATGAAAACACGGT
GGATGCACCTTTCTGTCTAAATGCGAATGAAAACAAGAGTGTGAAGATGATGATGCAAAAAGGCCTCT
ACAGAATTGGCTTCATAGAGGAGGTGAAGGCACAGATCCTGGAAATGAGGTACACCAAGGGGAAGCTC
AGCATGTTCGTGCTGCTGCCATCTCACTCTAAAGATAACCTGAAGGGTCTGGAAGAGCTTGAAAGGAA
AATCACCTATGAAAAAATGGTGGCTGGAGCAGCTCAGAAAACATGTCAGAAGAAATCGGTGGTCCTGT
CCTTCCCCCGGTTCACCCTGGAAGACAGCTATGATCTCAATTCCATTTTACAAGACATGGGCATTACG
GATATCTTTGATGAAACGAGGGCTGATCTTACTGGAATCTCTCCAAGTCCCAATTTGTACTTGTCAAA
AATTATCCACAAAACCTTTGTGGAGGTGGATGAAAACGGTACCCAGGCAGCTGCAGCCACTGGGGCTG
TTGTCTCGGAAAGGTCACTACGATCTTGGGTGGAGTTTAATGCCAACCACCCTTTTCTCTTTTTCATT
AGACACAACAAAACCCAAACCATTCTCTTTTATGGAAGGGTCTGCTCTCCTTAAAAGGGG

| | | | |
|---|---|---|---|
| NOV23a, CG54308-04 | | | |
| Protein Sequence | SEQ ID NO: 552 | 425 aa | MW at 48476.3kD |

MDSLVTANTKFCFDLFQEIGKDDRHKNIFFSPLSLSAALGMVRLGARSDSAHQIDEVLHFDEFSQNES
KEPDPCLKSNKQKVLADSSLEGQKKTTEPLDQQAGSLNNESGLVSCYFGQLLSJHDRIKTDYTLSIAN
RLYGEQEFPICQEYLDGVIQFYHTTIESVDFQKNPEKSRQEINFWVECQSQGKIKELFSKDAINAETV
LVLVNAVYFKAKWETYFDHENTVDAPFCLNANENKSVKMMMQKGLYRIGFIEEVKAQILEMRYTKGKL
SMFVLLPSHSKDNLKGLEELERKITYEKMVAWSSSENMSEESVVLSFPRFTLEDSYDLNSILQDMGIT
DIFDETRADLTGISPSPNLYLSKIIHKTFVEVDENGTQAAAATGAVVSERSLRSWVEFNANHPFLFFI
RHNKTQTILFYGRVCSP

| | | |
|---|---|---|
| NOV23b, CG54308-01 | SEQ ID NO: 553 | 1200 bp |
| DNA Sequence | ORF Start: ATG at 7 | ORF Stop: TAA at 1192 |

TTTACAATGGACTCTCTTGTTACAGCAAACACCAAATTTTGCTTTGATCTTTTTCAAGAGATAGGCAA
AGATGATCGTCATAAAAACATATTTTTCTCTCCCCTGAGCCTCTCAGCTGCCCTTGGTATGGTACGCT
GAAAGCAAAGAACCTGCTGGGTCCTTAAACAATGAGAGCGGACTGGTCAGCTGCTACTTTGGGCAGCT
TCTCTCCAAATTAGACAGGATCAAGACTGATTACACACTGAGTATTGCCAACAGGCTTTATGGAGAGC
AGGAATTCCCAATCTGTCAGGAATACTTAGATGGTGTGATTCAATTTTACCACACGACGATTGAAAGT
GTTGATTTCCAAAAAAACCCTGAAAAATCCAGACAAGAGATTAACTTCTGGGTTGAATGTCAATCCCA
AGGTAAATCAAGGACCTCTTCAGCAAGGACGCTATTAATGCTGAGACTGTGCTGGTACTGGGTGAATG
CTGTTTACTTCAAGGCCAAATGGGAAACATACTTTGACCATGAAAACACGGTGGATGCACCTTTCTGT
CTAAATCAGAATGAAAACAAGAGTGTGAAGATGATGACGCAAAAAGGCCTCTACAGAATTGGCTTCAT
AGAGGAGGTGAAGGCACAGATCCTGGAAATGAGGTACACCAAGGGGAAGCTCAGCATGTTCGTGCTGC
ATGGTGGCCTGGAGCAGCTCAGAAAACATGTCAGAAGAATCGGTGGTCCTGTCCTTCCCCCGGTTCAC
CCTGGAAGACAGCTATGATCTCAATTCCATTTTACAAGACATGGGCATTACGGATATCTTTGATGAAA
CGAGGGCTGATCTTACTGGAATCTCTCCAAGTCCCAATTTGTACTTGTCAAAAATTATCCACAAAACC
TTTGTGGAGGTGGATGAAAACGGTACCCAGGCAGCTGCAGCCACTGGGGCTGTTGTCTCGGAAAGGTC
AAACCATTCTCTTTTATGGCAGGGTCTGCTCTCCTTAAAAGGGG

| | | | |
|---|---|---|---|
| NOV23b, CG54308-01 | | | |
| Protein Sequence | SEQ ID NO: 554 | 395 aa | MW at 45176.6kD |

MDSLVTANTKFCFDLFQEIGKDDRHKNIFFSPLSLSAALGMVRLGARSDSAHQIDEVLHFNEFSQNES
KEPAGSLNNESGLVSCYFGQLLSKLDRIKTDYTLSIANRLYGEQEFPICQEYLDGVIQFYHTTIESVD
FQKNPEKSRQEINFWVECQSQGKIKDLFSKDAINAETVLVLVNAVYFKAKEWTYFDHENTVDAPFCLN
QNENKSVKMMTQKGLYRIGFIEEVKAQILEMRYTKGKLSMFVLLPSHSKDNLKGLEELERKITYEKMV
AWSSSENMSEESVVLSFPRFTLEDSYDLNSILQDMGITDIFDETRADLTGISPSPNLYLSKIIHKTFV
EVDENGTQAAAATGAVVSERSLRSWVEFNANHPFLFFIRHNKTQTILFYGRVCSP

| | | |
|---|---|---|
| NOV23c, CG54308-02 | SEQ ID NO: 555 | 1278 bp |
| DNA Sequence | ORF Start: at 1 | ORF Stop: end of sequence |

ACCATGGACTCTCTTGTTACAGCAAACACCAAATTTTGCTTTGATCTTTTTCAAGAGATAGGCAAAGA
TGATCGTCATAAAAACATATTTTTCTCTCCCCTGAGCCTCTCAGCTGCCCTTGGTATGGTACGCTTGG
GTGCTAGAAGTGACAGTGCACATCAGATTGATGAGGTACTACACTTCAACGAATTTTCCCAGAATGAA
AGCAAAGAACCTGACCCTTGTCTGAAAGCAACAAACAAAAAGTGCTGGACTGACAGCTCTCTGGAGGG
GCAGAAAAAAACGACAGAGCCTCTGGATCAGCAGGCTGGGTCCTTAAACAATGAGAGcGGACTGGTCA
GCTGCTACTTTGGGCAGCTTCTCTCCAAATTAGACAGGATCAAGACTGATTACACACTGAGTATTGCC
AACAGGCTTTATGGAGGGCAGGAATTCCCAATCTGTCAGGAATACTTAGATGGTGTGATTCAATTTTA
CCACACGACGATTGAAAGTGTTGATTTCCAAAAAAACCCTGAAAAATCCAGACAAGAGATTAACTTCT
GGGTTGAATGTCAATCCCAAGGTAAAATCAAGGAACTCTTCAGCAAGGACGCTATTAATGCTGAGACT
GTGCTGGTACTGGTGAATGCTGTTTACTTCAAGGCCAAATGGGAAACATACTTTGACCATGAAAACAC
GGTGGATGCACCTTTCTGTCTAAATGCGAATGAAAACAAGAGTGTGAAGATGATGACGCAAAAAGGCC
TCTACAGAATTGGCTTCATAGAGGAGGTGAAGGCACAGATCCTGGAAATGAGGTACACCAAGGGGAAG
CTCAGCATGTTCGTGCTGCTGCCATCTCACTCTAAAGATAACCTGAAGGGTCTGGAAGAGCTTGAAAG
GAAAATCACCTATGAAAAAATGGTGGCCTGGAGCAGCTCAGAAAACATGTCAGAAGAATCGGTGGTCC
TGTCCTTCCCCCGGTTCACCCTGGAAGACAGCTATGATCTCAATTCCATTTTACAAGACATGGGCATT
ACGGATATCTTTGATGAAACGAGGGCTGATCTTACTGGAATCTCTCCAAGTCCCAATTTGTACTTGTC
AAAAATTATCCACAAAACCTTTGTGGAGGTGGATGAAAACGGTACCCAGGCAGCTGCAGCCACTGGGG
CTGTTGTCTCGGAAAGGTCACTACGATCTTGGGTGGAGTTTAATGCCAACCACCCTTTTCTCTTTTTC
ATTAGACACAACAAAACCCAAACCATTCTCTTTTATGGCAGGGTCTGCTCTCCT

| | | | |
|---|---|---|---|
| NOV23c, CG54308-02 | | | |
| Protein Sequence | SEQ ID NO: 556 | 426 aa | MW at 48474.3kD |

TMDSLVTANTKFCFDLFQEIGKDDRHKNIFFSPLSLSAALGMVRLGARSDSAHQIDEVLHFNEFSQNE
SKEPDPCLKSNKQKVLADSSLEGQKKTTEPLDQQAGSLNNESGLVSCYFGQLLSKLDRIKTDYTLSIA
NRLYGGQEFPICQEYLDGVIQFYHTTIESVDFQKNPEKSRQEINFWVECQSQGKIKELFSKDAINAET
VLVLVNAVYFKAKWETYFDHENTVDAPFCLNANENKSVKMMTQKGLYRIGFIEEVKAQILEMRYTKGK
LSMFVLLPSHSKDNLKGLEELERKITYEKMVAWSSSENMSEESVVLSFPRFTLEDSYDLNSILQDMGI
TDIFDETRADLTGISPSPNLYLSKIIHKTFVEVDENGTQAAAATGAVVSERSLRSWVEFNANHPFLFF
IRHNKTQTILFYGRVCSP

| | | |
|---|---|---|
| NOV23d, CG54308-03 | SEQ ID NO: 557 | 1285 bp |
| DNA Sequence | ORF Start: ATG at 8 | ORF Stop: TAA at 1277 |

TTTTACAATGGACTCTCTTGTTACAGCAAACACCAAATTTTGCTTTGATCTTTTTCAAGAGATAGGCA
AAGATGATCGTCATAAAAACATATTTTTCTCTCCCCTGAGCCTCTCAGCTGCCCTTGGTATGGTACGC
TTGGGTGCTATAAGTGACAGTGCACATCACATTGATGAGGTACGTGTCCACTATTCCCAGAATGAAAG
CAAAGAACCTGACCCTTGTCTGAAAAGCAACAAACAAAAAGTGCTGGCTGACAGCTCTCTGGAGGGGC
AGAAAAAAACGACAGAGCCTCTGGATCAGCAGGCTGGGTCCTTAAACAATGGGAGCGGACTGGTCAGC
CAGGCTTTATGGAGAGCGGGAATTCCCAATCTGTCAGGAATACTTAGATGGTGTGATTCAATTTTACC
ACACGACGATTGAAAGTGTTGATTTCCAAAAAAACCCTGAAAAATCCAGACAAGAGATTAACTTCTGG
GTTGAATGTCAATCCCAAGGTAAAATCAAGGAACTCTTCAGCAAGGACGCTATTAATGCTGAGACTGT
GCTGGTACTGGTGAATGCTGTTTACTTCAAGGCCAAATGGGAAAACATACTTTGACCATGAAACACGG
TGGATGCACCTTTCTGTCTAAATGCGAATGAAAACAAGAGTGTGAAGATGATGACGCAAAAAGGCCTC
CAGCATGTTCGTGCTGCTGCCATCTCACTCTAAAGATAACCTGAAGGGTCTGGAAGAGCTTGAAAGGA
AAATCACCTATGAAAAAATGGTGGCCTGGAGCAGCTCAGAAAACATGTCAGAAGAATCGGTGGTCCTG
TCCTTCCCCCGGTTCACCCTGGAAGACAGCTATGATCTCAATTCCATTTTACAAGACATGGGCATTAC
GGATATCTTTGATGAAACGAGGGCTGATCTTACTGGAATCTCTCCAAGTCCCAATTTGTACTTGTCAA
AAATTATCCACAAAACCTTTGTGGAGGTGGATGAAAACGGTACCCAGGCAGCTGCAGCCACTGGGGCT
GTTGTCTCGGAAAGGTCACTACGATCTTGGGTGGAGTTTAATGCCAACCACCCTTTTCTCTTTTTCAT
TAGACACAACAAAACCCAAACCATTCTCTTTTATGGAAGGGTCTGCTCTCCTTAAAAGGGG

| | | | |
|---|---|---|---|
| NOV23d, CG54308-03 | | | |
| Protein Sequence | SEQ ID NO: 558 | 423 aa | MW at 48136.0kD |

MDSLVTANTKFCFDLFQEIGKDDRHKNIFFSPLSLSAALGMVRLGAISDSAHHIDEVRVHYSQNESKE
PDPCLKSNKQKVLADSSLEGQKKTTEPLDQQAGSLNNGSGLVSCYFGQLLSKLDRIKTDYTLSIADRL
LVNAVYFKAXWETYFDHENTVDAPFCLNANENKSVKMMTQKGLYRIGFIEEVKAQILEMRYTKGKLSM
FVLLPSHSKDNLKGLEELERKITYEKMVAWSSSENMSEESVVLSFPRFTLEDSYDLNSILQDMGITDI
FDETRADLTGISPSPNLYLSKIIHKTFVEVDENGTQAAAATGAVVSERSLRSWVEFNANHPFLFFIRH
NKTQTILFYGRVCSP

| | | |
|---|---|---|
| NOV23e, CG54308-05 | SEQ ID NO: 559 | 1196 bp |
| DNA Sequence | ORF Start: ATG at 60 | ORF Stop: TAA at 1155 |

CCAATCACCATTTTCTCTTTCTCCTTTTTTTTTGGTTTTAGATCGTTATAAGTTTTACAATGGACTCT
CTTGTTACAGCAAACACCAAATTTTGCTTTGATCTTTTTCAAGAGATAGGCAAAGATGATCGTCATAA
AAACATATTTTTCTCTCCCCTGAGCCTCTCAGCTGCCCTTGGTATGGTACGCTTGGGTGCTAGAAGTG
ACAGTGCACATCAGATTGATGAGGTACGTTCCTTAAACAATGAGAGCGGACTGGTCAGCTGCTACTTT
GGGCAGCTTCTCTCCAAATTAGACAGGATCAAGACTGATTACACACTGAGTATTGCCAACAGGCTTTA
TGGAGAGTCCAGCCTGGGAGACAAGAGCGAAACTCTGTCTCAAAAAAAAAAAAAAAAAATTATCTACA
CAAATGCTTTTGATACAATTCATACTCAGGATATTCTCTGGGATCTTTTTTTTAGGTAAATCAAGGAA
CTCTTCAGCAAGGACGCTATTAATGCTGAGACTGTGCTGGTACTGGTGATTGCTGTTTACTTCAAGGC
CAAATGGGAACATACTTTGACCATGAAAACACGGTGGATGCACCTTTCTGTCTLAATCAGAATGAAAA
ACAAGAGTGTGAAGATGATGACGCAAAAAGGCCTCTACAGAATTGGCTTCATAGAGGAGGTGAAGGCA
CAGATCCTGGAAATGAGGTACACCAAGGGGAAGCTCAGCATGTTCGTGCTGCTGCCATCTCACTCTAA
AGATAACCTGAAGGGTATCACCTATGAAAAAATGGTGGCCTGGAGCAGCTCAGAAAACATGTCAGAAG
AATCGGTGGTCCTGTCCTTCCCCCGGTTCACCCTGGAAGACAGCTATGATCTCAATTCCATTTTACAA
GACATGGGCATTACGGATATCTTTGATGAAACGAGGGCTGATCTTACTGGAATCTCTCCAAGTCCCAA
TTTGTACTTGTCAAAAATTATCCACAAAACCTTTGTGGAGGTGGATGAAAACGGTACCCAGGCAGCTG
CAGCCACTGGGGCTGTTGTCTCGGAAAGGTCACTACGATCTTGGGTGGAGTTTAATGCCAACCACCCT
TTTCTCTTTTTCATTAGACACAACAAAACCCAAACCATTCTCTTTTATGGCAGGGTCTGCTCTCCTTA
AAAGGGGAGCAGTGTCTAGTACTTTGGAGCTGGAGGAAAA

| | | | |
|---|---|---|---|
| NOV23e, | | | |
| Protein Sequence | SEQ ID NO: 560 | 365 aa | MW at 41476.8kD |

MDSLVTANTKFCFDLFQEIGKDDRhKNIFFSPLSLSAALGMVRLGARSDSAHQIDEVRSLNNESGLVS
CYFGQLLSKLDRIKTDYTLSIANPLYGESSLGDKSETLSQKKKKKIIYTNAFDTIHTQDILWDLFLGK
IKELFSKDAINAETVLVLVNAVYFKACWETYFDHENTVDAPFCLNQNENKSVKMMTQKGLYRIGFIEE
VKAQILEMRYTKGKLSMFVLLPSHSKDNLKGITYEKMVAWSSSENMSEESVVLSFPRFTLEDSYDLNS
ILQDMGITDIFDETPADLTGISPSPNLYLSKIIHKTFVEVDENGTQAAAATGAVVSERSLRSWVEFNA
NHPFLFFIRHNKTQTILFYGRVCSP

A ClustalW comparison of the above protein sequences yields the following sequence alignment shown in Table 23B.

TABLE 23B

Comparison of the NOV23 protein sequences.

| | |
|---|---|
| NOV23a | -MDSLVTANTKFCFDLFQEIGKDDRHKNIFFSPLSLSAALGMVRLGARSDSAHQIDEVLH |
| NOV23b | -MDSLVTANTKFCFDLFQEIGKDDRHKNIFFSPLSLSAALGMVRLGARSDSAHQIDEVLH |
| NOV23c | TMDSLVTANTKFCFDLFQEIGKDDRHKNIFFSPLSLSAALGMVRLGARSDSAHQIDEVLH |
| NOV23d | -MDSLVTANTKFCFDLFQEIGKDDRHKNIFFSPLSLSAAIGMVRLGAISDSAHHIDEVR- |
| NOV23e | -MDSLVTANTKFCFDLFQEIGKDDRHKNIFFSPLSLSAALGMVRLGARSDSAHQIDEVR- |
| NOV23a | FDEFSQNESKEPDPCLKSNKQKVLADSSLEGQKKTTEPLDQQAGSLNNESGLVSCYFGQL |
| NOV23b | FNEFSQNESKEP------------------------------AGSLNNESGLVSCYFGQL |
| NOV23c | FNEFSQNESKEPDPCLKSNKQKVLADSSLEGQKKTTEPLDQQAGSLNNESGLVSCYFGQL |
| NOV23d | -VHYSQNESKEPDPCLKSNKQKVLADSSLEGQKKTTEPLDQQAGSLNNGSGLVSCYFGQL |
| NOV23e | --------------------------------------------SLNNESGLVSCYFGQL |
| NOV23a | LSKLDRIKTDYTLSIANRLYGEQEFPICQEYLDGVIQFYHTTIESVDFQKNPEKSRQEIN |
| NOV23b | LSKLDRIKTDYTLSIANRLYGEQEFPICQEYLDGVIQFYHTTIESVDFQKNPEKSRQEIN |
| NOV23c | LSKLDRIKTDYTLSIANRLYGGQEFPICQEYLDGVIQFYHTTIESVDFQKNPEKSRQEIN |
| NOV23d | LSKLDRIKTDYTLSIADRLYGEREFPICQEYLDGVIQFYHTTIESVDFQKNPEKSRQEIN |
| NOV23e | LSKLDRIKTDYTLSIANRLYGESSLGDKSETLS------QKKKKKIIYThAFDTIHTQDI |
| NOV23a | FWVECQSQGKIKELFSKDAINAETVLVLVNAVYFKAKWETYFDHENTVDAPFCLNANENK |
| NOV23b | FWVECQSQGKIKDLFSKDAINAETVLVLVNAVYFKAKWETYFDHENTVDAPFCLNQNENK |
| NOV23c | FWVECQSQGKIKELFSKDAINAETVLVLVNAVYFKAKWETYFDHENTVDAPFCLNANENK |
| NOV23d | FWVECQSQGKIKELFSKDAINAETVLVLVNAVYFKAKWETYFDHENTVDAPFCLNANENK |
| NOV23e | LWDLF--LGKIKELFSKDAINAETVLVLVNAVYFKAKWETYFDHENTVDAPFCLNQNENK |
| NOV23a | SVKMMMQKGLYRIGFIEEVKAQILEMRYTKGKLSMFVLLPSHSKDNLKGLEELERKITYE |
| NOV23b | SVKMMTQKGLYRIGFIEEVKAQILENRYTKGKLSMFVLLPSHSKDNLKGLEELERKITYE |
| NOV23c | SVKNMTQKGLYRIGFIEEVKAQILEMRYTKGKLSMFVLLPSHSKDNLKGLEELERKITYE |
| NOV23d | SVKMMTQKGLYRIGFIEEVKAQILEMRYTKGKLSMFVLLPSHSKDNLKGLEELERKITYE |
| NOV23e | SVKMMTQKGLYRIGFIEEVKAQILEMRYTKGKLSMFVLLPSHSKDNLKG-------ITYE |
| NOV23a | KMVAWSSSENMSEESVVLSFPRFTLEDSYDLNSILQDMGITDIFDETRADLTGISPSPNL |
| NOV23b | KMVAWSSSENMSEESVVLSFPRFTLEDSYDLNSILQDMGITDIFDETRADLTGISPSPNL |
| NOV23c | KMVAWSSSENMSEESVVLSFPRFTLEDSYDLNSILQDMGITDIFDETRADLTGISPSPNL |
| NOV23d | KMVAWSSSENMSEESVVLSFPRFTLEDSYDLNSILQDMGITDIFDETRADLTGISPSPNL |
| NOV23e | KMVAWSSSENMSEESVVLSFPRFTLEDSYDLNSILQDMGITDIFDETRADLTGISPSPNL |
| NOV23a | YLSKIIHKTFVEVDENGTQAAAATGAVVSERSLRSWVEFNANHPFLFFIRHNKTQTILFY |
| NOV23b | YLSKIIHKTFVEVDENGTQAAAATGAVVSERSLRSWVEFNANHPFLFFIRHNKTQTILFY |
| NOV23c | YLSKIIHKTFVEVDENGTQAAAATGAVVSERSLRSWVEFNANHPFLFFIRHNKTQTILFY |
| NOV23d | YLSKIIHKTFVEVDENGTQAAAATGAVVSERSLRSWVEFNANHPFLFFIRHNKTQTILFY |
| NOV23e | YLSKIIHKTFVEVDENGTQAAAATGAVVSERSLRSWVEFNANHPFLFFIPHNKTQTILFY |
| NOV23a | GRVCSP |
| NOV23b | GRVCSP |
| NOV23c | GRVCSP |
| NOV23d | GRVCSP |
| NOV23e | GRVCSP |
| NOV23a | (SEQ ID NO: 552) |
| NOV23b | (SEQ ID NO: 554) |
| NOV23c | (SEQ ID NO: 556) |
| NOV23d | (SEQ ID NO: 558) |
| NOV23e | (SEQ ID NO: 560) |

Further analysis of the NOV23a protein yielded the following properties shown in Table 23C.

TABLE 23C
Protein Sequence Properties NOV23a

| | |
|---|---|
| SignalP analysis: | No Known Signal Sequence Predicted |
| PSORT II analysis: | |

| PSG: | a new signal peptide prediction method | |
|---|---|---|
| | N-region: | length 10; pos. chg 1; neg. chg 1 |
| | H-region: | length 3; peak value −2.92 |
| | PSG score: | −7.33 |

| | |
|---|---|
| GvH: | von Heijne's method for signal seq. recognition |
| | GvH score (threshold: −2.1): −2.16 |
| | possible cleavage site: between 40 and 41 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 1 | |
| | Number of TMS(s) for threshold 0.5: 0 | |
| | PERIPHERAL | Likelihood = 3.07 (at 197) |

| | ALOM score: 0.42 (number of TMSs: 0) | | | |
|---|---|---|---|---|
| MITDISC: | discrimination of mitochondrial targeting seq | | | |
| | R content: | 0 | Hyd Moment (75): | 7.00 |
| | Hyd Moment (95): | 3.76 | G content: | 0 |
| | D/E content: | 2 | S/T content: | 3 |

| | |
|---|---|
| | Score: −6.29 |
| Gavel: | prediction of cleavage sites for mitochondrial preseq |
| | cleavage site motif not found |

NUCDISC: discrimination of nuclear localization signals

| | |
|---|---|
| | pat4: none |
| | pat7: none |
| | bipartite: none |
| | content of basic residues: 10.8% |
| | NLS Score: −0.47 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear |
| | discrimination |
| | Prediction: cytoplasmic |
| | Reliability: 70.6 |
| COIL: | Lupas's algorithm to detect coiled-coil regions |
| | total: 0 residues |

Final Results (k = 9/23):

47.8%: mitochondrial
30.4%: cytoplasmic
17.4%: nuclear
4.3%: vacuolar

>> prediction for CG54308-04 is mit (k = 23)

A search of the NOV23a protein against the Geneseq database, a proprietary database that contains sequences published in patents and patent publication, yielded several homologous proteins shown in Table 23D.

TABLE 23D
Geneseq Results for NOV23a

| | | NOV23a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABG70630 | Amino acid sequence for novel human | 1 . . . 425 | 423/425 (99%) | 0.0 |
| | thypin - Homo sapiens, 425 aa. | 1 . . . 425 | 424/425 (99%) | |
| | [WO200272769-A2, 19-SEP-2002] | | | |
| ABB53280 | Human polypeptide #20 - Homo | 1 . . . 425 | 392/425 (92%) | 0.0 |
| | sapiens, 394 aa. [WO200181363-A1, | 1 . . . 394 | 393/425 (92%) | |
| | 01-NOV-2001] | | | |
| AAM47207 | Human NOV1 protein - Homo sapiens, | 1 . . . 425 | 391/425 (92%) | 0.0 |
| | 395 aa. [WO200174851-A2, 11-OCT-2001] | 1 . . . 395 | 393/425 (92%) | |
| ABG28280 | Novel human diagnostic protein | 1 . . . 394 | 388/394 (98%) | 0.0 |
| | #28271 - Homo sapiens, 617 aa. | 109 . . . 502 | 389/394 (98%) | |
| | [WO200175067-A2, 11-OCT-2001] | | | |
| ABG70294 | Human novel polypeptide #10 - Homo | 1 . . . 425 | 327/425 (76%) | e−172 |
| | sapiens, 365 aa. [WO200257452-A2, | 1 . . . 365 | 332/425 (77%) | |
| | 25-JUL-2002] | | | |

In a BLAST search of public sequence databases, the NOV23a protein was found to have homology to the proteins shown in the BLASTP data in Table 23E.

TABLE 23E
Public BLASTP Results for NOV23a

| | | NOV23a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96P63 | Serpin B12 - Homo sapiens | 1 . . . 425 | 403/425 (94%) | 0.0 |
| | (Human), 405 aa. | 1 . . . 405 | 404/425 (94%) | |
| CAD13083 | Sequence 1 from Patent | 1 . . . 425 | 391/425 (92%) | 0.0 |
| | WO0174851 - Homo sapiens | 1 . . . 395 | 393/425 (92%) | |
| | (Human), 395 aa. | | | |
| Q9D7P9 | 2300003F07Rik protein (Weakly | 1 . . . 425 | 303/425 (71%) | e−174 |
| | similar to serpin B12) - Mus | 1 . . . 423 | 354/425 (83%) | |
| | musculus (Mouse), 423 aa. | | | |
| I38202 | leupin precursor - human, 390 aa. | 1 . . . 425 | 196/426 (46%) | 4e−95 |
| | | 1 . . . 390 | 267/426 (62%) | |
| P48594 | Squamous cell carcinoma antigen 2 | 1 . . . 425 | 196/426 (46%) | 4e−95 |
| | (SCCA-2) (Leupin) - Homo sapiens | 1 . . . 390 | 267/426 (62%) | |
| | (Human), 390 aa. | | | |

PFam analysis predicts that the NOV23a protein contains the domains shown in the Table 23F.

TABLE 23F
Domain Analysis of NOV23a

| | | Identities/ | |
|---|---|---|---|
| Pfam | NOV23a Match | Similarities for | |
| Domain | Region | the Matched Region | Expect Value |
| serpin | 1 . . . 425 | 161/441 (37%) | 1.9e−147 |
| | | 350/441 (79%) | |

### Example 24

The NOV24 clone was analyzed, and the nucleotide and encoded polypeptide sequences are shown in Table 24A.

TABLE 24A

NOV24 Sequence Analysis

| | | |
|---|---|---|
| NOV24a, CG54764-02 | SEQ ID NO: 561 | 339 bp |
| DNA Sequence | ORF Start: ATG at 1 | ORF Stop: TGA at 337 |

ATGAGCCCCTTTGGCAGTCTGGCGAAGCTCTTGGGTCCTTCTCAGATTGCATGGTGGTGCATCACGAC
CCTGTGCTGTTTTCCAGAGAGGTATGCTGGACGGGACCATAACAGCTGCAAACTCTCCCAGAGGGGGT
TCCTAAACTTCATGAACACTGTACTGGTTGCCTTCACAAAGAACCAGAAGGGCTCTGGTGCCCTTGAC
TGCATGATGAAGAAACTGGACTTCAACTGTGATGGGCAGCTAGATTTTCAGGACTTTCTCAGTCTTAC
TGATGGTGTAGCTGTGGCTTGCCCTGACTCCTTCATCCCGGCTGGCCATGCCCATGAGAGAATCTGA

| | | | |
|---|---|---|---|
| NOV24a, CG54764-02 | | | |
| Protein Sequence | SEQ ID NO: 562 | 112 aa | MW at 12368.1kD |

MSPFGSLAKLLGPSQIAWWCITTLCCFPERYAGRDHNSCKLSQRGFLNFMNTVLVAFTKNQKGSGALD
CMMKKLDFNCDGQLDFQDFLSLTDGVAVACPDSFIPAGHAHERI

| | | |
|---|---|---|
| NOV24b, CG54764-01 | SEQ ID NO: 563 | 348 bp |
| DNA Sequence | ORF Start: ATG at 5 | ORF Stop: TGA at 341 |

TGCCATGAGCCCCTTTGGCAGTCTGGCGAAGCTCTTGGGTCCTTCTCAGATTGCATGGTGGTGCATCA
CGACCTGTGCTGTTTTCCAGAGAGGGTATGCTGGACGGGACCATAACAGCTGCAAACTCTCCCAGAGG
GGGTTCCTAAACTTCATGAACACTGTACTGGTTGCCTTCACAAAGAACCAGAAGGGCTCTGGTGCCCT
TGACTGCATGATGAAGAAACTGGACTTCAACTGTGATGGGCAGCTAGATTTTCAGGACTTTCTCAGTC
TTACTGATGGTGTAGCTGTGGCTTGCCCTGACTCCTTCATCCCGGCTGGCCATGCCCATGAGAGAATC
TGAGGTGC

| | | | |
|---|---|---|---|
| NOV24b, 0G54764-01 | | | |
| Protein Sequence | SEQ ID NO: 564 | 112 aa | MW at 12281.0kD |

MSPFGSLAKLLGPSQIAWWCITTCAVFQRGYAGRDHNSCKLSQRGFLNFMNTVLVAFTKNQKGSGALD
CMMKKLDFNCDGQLDFQDFLSLTDGVAVACPDSFIPAGHAHERI

A ClustalW comparison of the above protein sequences yields the following sequence alignment shown in Table 24B.

TABLE 24B

Comparison of the NOV24 protein sequences.

| | |
|---|---|
| NOV24a | MSPFGSLAKLLGPSQIAWWCITTLCCFPERYAGRDHNSCKLSQRGFLNFMNTVLVAFTKN |
| NOV24b | MSPFGSLAKLLGPSQIAWWCITTCAVFQRGYAGRDHNSCKLSQRGFLNFMNTVLVAFTKN |
| NOV24a | QKGSGALDCMMKKLDFNCDGQLDFQDFLSLTDGVAVACPDSFIPAGHAHERI |
| NOV24b | QKGSGALDCMMKKLDFNCDGQLDFQDFLSLTDGVAVACPDSFIPAGHAHERI |
| NOV24a | (SEQ ID NO: 562) |
| NOV24b | (SEQ ID NO: 564) |

Further analysis of the NOV24a protein yielded the following properties shown in Table 24C.

TABLE 24C
Protein Sequence Properties NOV24a

| | |
|---|---|
| SignalP analysis: | Cleavage site between residues 33 and 34 |
| PSORT II analysis: | |

| PSG: | a new signal peptide prediction method | |
|---|---|---|
| | N-region: | length 9; pos. chg 1; neg. chg 0 |
| | H-region: | length 19; peak value 9.43 |
| | PSG score: | 5.03 |

| | |
|---|---|
| GvH: | von Heijne's method for signal seq. recognition |
| | GvH score (threshold: −2.1): −5.43 |
| | possible cleavage site: between 28 and 29 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 0 | |
| | number of TMS(s) . . . fixed | |
| | PERIPHERAL | Likelihood = 3.18 (at 87) |

| | ALOM score: 3.18 (number of TMSs: 0) | | | |
|---|---|---|---|---|
| MTOP: | Prediction of membrane topology (Hartmann et al.) | | | |
| | Center position for calculation: 6 | | | |
| | Charge difference: 0.0 C(1.0)-N(1.0) | | | |
| | N >= C: N-terminal side will be inside | | | |
| MITDISC: | discrimination of mitochondrial targeting seq | | | |
| | R content: | 0 | Hyd Moment (75): | 0.38 |
| | Hyd Moment (95): | 3.15 | G content: | 2 |
| | D/E content: | 1 | S/T content: | 5 |

| | |
|---|---|
| | Score: −5.58 |
| Gavel: | prediction of cleavage sites for mitochondrial preseq |
| | cleavage site motif not found |

NUCDISC: discrimination of nuclear localization signals

| | |
|---|---|
| | pat4: none |
| | pat7: none |
| | bipartite: none |
| | content of basic residues: 8.9% |
| | NLS Score: −0.47 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear |
| | discrimination |
| | Prediction: cytoplasmic |
| | Reliability: 89 |
| COIL: | Lupas's algorithm to detect coiled-coil regions |
| | total: 0 residues |

Final Results (k = 9/23):

```
43.5%: cytoplasmic
21.7%: mitochondrial
21.7%: nuclear
  4.3%: extracellular, including cell wall
  4.3%: vacuolar
  4.3%: endoplasmic reticulum
```

>> prediction for CG54764-02 is cyt (k = 23)

A search of the NOV24a protein against the Geneseq database, a proprietary database that contains sequences published in patents and patent publication, yielded several homologous proteins shown in Table 24D.

TABLE 24D
Geneseq Results for NOV24a

| | | NOV24a | Identities/ | | |
|---|---|---|---|---|---|
| | | Residues/ | Similarities for | | |
| Geneseq | Protein/Organism/Length | Match | the Matched | | Expect |
| Identifier | [Patent #, Date] | Residues | Region | | Value |
| AAE16342 | Human calgizzarin-like-like protein, | 1 . . . 112 | 106/112 | (94%) | 4e−59 |
| | POLY6 - Homo sapiens, 112 aa. | 1 . . . 112 | 106/112 | (94%) | |
| | [WO200185767-A2, 15-NOV-2001] | | | | |
| AAG65905 | Amino acid sequence of GSK gene Id | 7 . . . 112 | 100/106 | (94%) | 2e−57 |
| | 35812 - Homo sapiens, 106 aa. | 1 . . . 106 | 104/106 | (97%) | |
| | [WO200172961-A2, 04-OCT-2001] | | | | |
| AAG65904 | Amino acid sequence of GSK gene Id | 20 . . . 108 | 88/89 | (98%) | 2e−47 |
| | 35812 - Homo sapiens, 114 aa. | 26 . . . 113 | 88/89 | (98%) | |
| | [WO200172961-A2, 04-OCT-2001] | | | | |
| AAB58356 | Lung cancer associated polypeptide | 6 . . . 111 | 54/106 | (50%) | 6e−22 |
| | sequence SEQ ID 694 - Homo sapiens, | 29 . . . 133 | 73/106 | (67%) | |
| | 134 aa. [WO200055180-A2, 21-SEP-2000] | | | | |
| ABG96407 | Human ovarian cancer marker M471 - | 7 . . . 111 | 54/105 | (51%) | 7e−22 |
| | Homo sapiens, 105 aa. | 1 . . . 104 | 72/105 | (68%) | |
| | [WO200271928-A2, 19-SEP-2002] | | | | |

In a BLAST search of public sequence databases, the NOV24a protein was found to have homology to the proteins shown in the BLASTP data in Table 24E.

TABLE 24E
Public BLASTP Results for NOV24a

| | | NOV24a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| P50543 | Calgizzarin (Endothelial monocyte- | 20 . . . 103 | 52/84 | (61%) | 9e−24 |
| | activating polypeptide) (EMAP) - Mus | 8 . . . 91 | 65/84 | (76%) | |
| | musculus (Mouse), 98 aa. | | | | |
| P24480 | Calgizzarin (S100C protein) - | 20 . . . 111 | 53/92 | (57%) | 1e−22 |
| | Oryctolagus cuniculus (Rabbit), 102 | 10 . . . 101 | 66/92 | (71%) | |
| | aa. | | | | |
| BAC56502 | Similar to S100 calcium-binding | 7 . . . 110 | 55/104 | (52%) | 2e−22 |
| | protein A11 - Bos taurus (Bovine), 103 | 1 . . . 103 | 73/104 | (69%) | |
| | aa (fragment). | | | | |
| P31950 | Calgizzarin (S100C protein) - Sus | 20 . . . 103 | 50/84 | (59%) | 2e−21 |
| | scrofa (Pig), 99 aa. | 11 . . . 94 | 63/84 | (74%) | |
| P31949 | Calgizzarin (S100C protein) (MLN 70) - | 7 . . . 111 | 54/105 | (51%) | 2e−21 |
| | Homo sapiens (Human), 105 aa. | 1 . . . 104 | 72/105 | (68%) | |

PFam analysis predicts that the NOV24a protein contains the domains shown in the Table 24F.

TABLE 24F
Domain Analysis of NOV24a

| | | Identities/ | |
|---|---|---|---|
| Pfam | NOV24a | Similarities for | Expect |
| Domain | Match Region | the Matched Region | Value |
| S_100 | 20 . . . 60 | 11/44 (25%) | 0.23 |
| | | 28/44 (64%) | |
| efhand | 66 . . . 94 | 7/29 (24%) | 0.22 |
| | | 22/29 (76%) | |

### Example 25

The NOV25 clone was analyzed, and the nucleotide and encoded polypeptide sequences are shown in Table 25A.

TABLE 25A

NOV25 Sequence Analysis

| | | |
|---|---|---|
| NOV25a, CG55033-04 | SEQ ID NO: 565 | 1722 bp |
| DNA Sequence | ORF Start: ATG at 1 | ORF Stop: TAG at 1591 |

ATGAAGCTGTGGATTCATCTCTTTTATTCATCTCTCCTTGCCTGTATATCTTTACACTCCCAAACTCC
AGTGCTCTCATCCAGAGGCTCTTGTGATTCTCTTTGCAATTGTGAGGAAAAAGATGGCACAATGCTAA
TAAATTGTGAAGCAAAAGGTATCAAGATGGTATCTGAAATAAGTGTGCCACCATCACGACCTTTCCAA
CTAAGCTTATTAAATAACGGCTTGACGATGCTTCACACAAATGACTTTTCTGGGCTTACCAATGCTAT
TTCAATACACCTTGGATTTAACAATATTGCAGATATTGAGATAGGTGCATTTAATGGCCTTGGCCTCC
TGAAACAACTTCATATCAATCACAATTCTTTAGAAATTCTTAAAGAGGATACTTTCCATGGACTGGAA
CAACAGACTCAAAGTGTTAATTTTAAATGACAATGCTATTGAGAGTCTTCCTCCAAACATCTTCCGAT
TTGTTCCTTTAACCCATCTAGATCTTCGTGGAAATCAATTACAAACATTGCCTTATGTTGGTTTTCTC
GAACACATTGGCCGAATATTGGATCTTCAGTTGGAGGACAACAAATGGGCCTGCAATTGTGACTTATT
GCAGTTAAAAACTTGGTTGGAGAACATGCCTCCACAGTCTATAATTGGTGATGTTGTCTGCAACAGCC
CTCCATTTTTTAAAGGAAGTATACTCAGTAGACTAAAGAAGGAATCTATTTGCCCTACTCCACCAGTG
TATGAAGAACATGAGGATCCTTCAGGATCATTACATCTGGCAGCAACATCTTCAATAAATGATAGTCG
CATGTCAACTAAGACCACGTCCATTCTAAAACTACCCACCAAAGCACCAGGTTTGATACCTTATATTA
CAAAGCCATCCACTCAACTTCCAGGACCTTACTGCCCTATTCCTTGTAACTGCAAAGTCCTATCCCCA
TCAGGACTTCTAATACATTGTCAGGAGCGCAACATTGAAAGCTTATCAGATCTGAGACCTCCTCCGCA
AAATCCTAGAAAGCTCATTCTAGCGGGAAATATTATTCACAGTTTAATGAAGAGTCCATCCTTTGGTC
CAAAGCATCTGGAAGAGGAAGAAGAGAGGAATGAGAAAGAAGGAAGTGATGCAAAACATCTCCAAAGA
AGTCTTTTGGAACAGGAAAATCATTCACCACTCACAGGGTCAAATATGAAATACAAAACCACGAACCA
ATCAACAGAATTTTTATCCTTCCAAGATGCCAGCTCATTGTACAGAAACATTTTAGAAAAAGAAAGGG
AACTTCAGCAACTGGGAATCACAGAATACCTAAGGAAAAACATTGCTCAGCTCCAGCCTGATATGGAG
GGTATTAGTGGAACAGACAAAAAATGAGTATTTTGAACTTAAAGCTAATTTACATGCTGAACCTGACT
ATTTAGAAGTCCTGGAGCAGCAAACATAGATGGAGAGTTTGAGGGCTTTCGCAGAAATGCTGTGATTC
TGTTTTAAGTCCATACCTTGTAAATTAGTGCCTTACGTGAGTGTGTCATCCATCAGAACCTAAGCACA
GCAGTAAACTATGGAGAAAAAA

| | | | |
|---|---|---|---|
| NOV25a, CG55033-04 | | | |
| Protein Sequence | SEQ ID NO: 566 | 530 aa | MW at 59730.7kD |

MKLWIHLFYSSLLACISLHSQTPVLSSRGSCDSLCNCEEKDGTMLINCEAKGIKMVSEISVPPSRPFQ
LSLLNNGLTMLHTNDFSGLTNAISIHLGFNNIADIEIGAFNGLGLLKQLHINHNSLEILKEDTFHGLE
NLEFLQADNNFITVIEPSAFSKLNRLKVLILNDNAIESLPPNIFRFVPLTHLDLRGNQLQTLPYVGFL
EHIGRILDLQLEDNKWACNCDLLQLKTWLENMPPQSIIGDVVCNSPPFFKGSILSRLKKESICPTPPV
YEEHEDPSGSLHLAATSSINDSRMSTKTTSILKLPTKAPGLIPYITKPSTQLPGPYCPIPCNCKVLSP
SGLLIHCQERNIESLSDLRPPPQNPRKLILAGNIIHSLMKSPSFGPKHLEEEEERNEKEGSDAKHLQR
SLLEQENHSPLTGSNMKYKTTNQSTEFLSFQDASSLYRNILEKERELQQLGITEYLRNKIAQLQPDME
AHYPGAHEELKLMETLMYSRPRKVLVEQTKNEYFELKANLHAEPDYLEVLEQQT

| | | |
|---|---|---|
| NOV25b, CG55033-05 | SEQ ID NO: 567 | 2058 bp |
| Sequence | ORF Start: ATG at 1 | ORF Stop: TAG at 1927 |

ATGAAGCTGTGGATTCATCTCTTTTATTCATCTCTCCTTGCCTGTATATCTTTACACTCCCAAACTCC
AGTGCTCTCATCCAGAGGCTCTTGTGATTCTCTTTGCAATTGTGAGGAAAAAGATGGCACAATGCTAA
TAAATTGTGAAGCAAAAGGTATCAAGATGGTATCTGAAATAAGTGTGCCACCATCACGACCTTTCCAA
CTAAGCTTATTAAATAACGGCTTGACGATGCTTCACACAAATGACTTTTCTGGGCTTACCAATGCTAT
TTCAATACACCTTGGATTTAACAATATTGCAGATATTGAGATAGGTGCATTTAATGGCCTTGGCCTCC
TGAAACAACTTCATATCAATCACAATTCTTTAGAAATTCTTAAAGAGGATACTTTCCATGGACTGGAA
AACCTGGAATTCCTGCAAGCAGATAACAATTTTATCACAGTGATTGAACCAAGTGCCTTTAGCAAGCT
CAACAGACTCAAAGTGTTAATTTTAAATGACAATGCTATTGAGAGTCTTCCTCCAAACATCTTCCGAT
TTGTTCCTTTAACCCATCTAGATCTTCGTGGAAATCAATTACAAACATTGCCTTATGTTGGTTTTCTC
GCAGTTAAAACTTGGTTGGAGAACATGCCTCCACAGTCTATAATTGGTGATGTTGTCTGCAAACAGCC
CTCCATTTTTTAAAGGAAGTATACTCAGTAGACTAAAGAAGGAATCTATTTGCCCTACTCCACCAGTG
TATGAAGAACATGAGGATCCTTCAGGATCATTACATCTGGCAGCAACATCTTCAATAAATGATAGTCG
CATGTCAACTAAGACCACGTCCATTCTAAACTACCCACCAAAGCACCACAGATTGACCTTGAGAGATA
ACCCCTGGGACTGCTCCTGTGACCTGGTTGGACTGCAGCAATGGATACAAAAGTTAAGCAAGAACACA
GTGACAGATGACATCCTCTGCACTTCCCCCGGGCATCTCGACAAAAAGGAATTGAAAGCCCTAAATAG
CCACTCCTGCAACAACAACAAATACGGCTGATACTATTTTACGATCTCTTACGGACGCTGTGCCACTG
TCTGTTCTAATATTGGGACTTCTGATTATGTTCATCACTATTGTTTTCTGTGCTGCAGGGATAGTGGT
TCTTGTTCTTCACCGCAGGAGAAGATACAAAAAGAAACAAGTAGATGAGCAAATGAGAGACAACAGTC
CTGTGCATCTTCAGTACAGCATGTATGGCCATALAACCACTCATCACACTACTGAAAGACCCTCTGCC
TCACTCTATGAACAGCACATGGTGAGCCCCATGGTTCATGTCTATAGAAGTCCATCCTTTGGTCCAAA
GCATCTGGAAGAGGAAGAAGAGAGGAATGAGAAAGAAGGAAGTGATGCAAAACATCTCCAAAGAAGTC
TTTTGGAACAGGAAAATCATTCACCACTCACAGGGTCAAATATGAAATACAAAACCACGAACCAATCA
ACAGAATTTTTATCCTTCCAAGATGCCAGCTCATTGTACAGAAACATTTTAGAAAAAGAAAGGGAACT
TCAGCAACTGGGAATCACAGAATACCTAAGGAAAAACATTGCTCAGCTCCAGCCTGATATGGAGGCAC
ATTATCCTGGAGCCCACGAAGAGCTGAAGTTAATGGAAACATTAATGTACTCACGTCCAAGGAAGGTA
TTAGTGGAACAGACAAAAAATGAGTATTTTGAACTTAAAGCTAATTTACATGCTGAACCTGACTATTT
AGAAGTCCTGGAGCAGCAAACATAGATGGAGAGTTTGAGGGCTTTCGCAGAAATGCTGTGATTCTGTT
TTAAGTCCATACCTTGTAAATTAGTGCCTTACGTGAGTGTGTCATCCATCAGAACCTAAGCACAGCAG
TAAACTATGGAGAAAAAA

| | | | |
|---|---|---|---|
| NOV25b, CG55033-05 | | | |
| Protein Sequence | SEQ ID NO: 568 | 642 aa | MW at 72606.2kD |

MKLWIHLFYSSLLACISLHSQTPVLSSRGSCDSLCNCEEKDGTMLINCEAKGIKMVSEISVPPSRPFQ
LSLLNNGLTMLHTNDFSGLTNAISIHLGFNNIADIEIGAFNGLGLLKQLHINHNSLEILKEDTFHGLE
NLEFLQADNNFITVIEPSAFSKLNRLKVLILNDNAIESLPPNIFRFVPLTHLDLRGNQLQTLPYVGFL
EHIGRILDLQLEDNKWACNCDLLQLKTWLENMPPQSIIGDVVCNSPPFFKGSILSRLKKESICPTPPV
YEEHEDPSGSLHLAATSSINDSRMSTKTTSILKLPTKAPQIDLEDNPWDCSCDLVGLQQWIQKLSKNT
VTDDILCTSPGHLDKKELKALNSEILCPGLVNNPSMPTQTSYLMVTTPATTTNTADTILRSLTDAVPL
SVLILGLLIMFITIVFCAAGIVVLVLHRRRRYKKKQVDEQMRDNSPVHLQYSMYGHKTTHHTTERPSA
SLYEQHMVSPMVHVYRSPSFGPKHLEEEEERNEKEGSDAKHLQRSLLEQENHSPLTGSNMKYKTTNQS
TEFLSFQDASSLYRNILEKERELQQLGITEYLRKNIAQLQPDMEAHYPGAHEELKLMETLMYSRPRKV
LVEQTKNEYFELKANLHAEPDYLEVLEQQT

| | | |
|---|---|---|
| NOV25c, CG55033-01 | SEQ ID NO: 569 | 2397 bp |
| DNA Sequence | ORF Start: ATG at 1 | ORF Stop: TAG at 2395 |

ATGCTAATAAATTGTGAAGCAAAAGGTATCAAGATGGTATCTGAAATAAGTGTGCCACCATCACGACC
TTTCCAACTAAGCTTATTAAATAACGGCTTGACGATGCTTCACACAAATGACTTTTCTGGGCTTACCA
ATGCTATTTCAATACACCTTGGATTTAACAATATTGCAGATATTGAGATAGGTGCATTTAATGGCCTT
GGCCTCCTGAAACAACTTCATATCAATCACAATTCTTTAGAAATTCTTAAAGAGGATACTTTCCATGG
ACTGGAAAACCTGGAATTCCTGCAAGCAGATAACAATTTTATCACAGTGATTGAACCAAGTGCCTTTA
GCAAGCTCAACAGACTCAAAGTGTTAATTTTAAATGACAATGCTATTGAGAGTCTTCCTCCAAACATC
TTCCGATTTGTTCCTTTAACCCATCTAGATCTTCGTGGAAATCAATTACAAACATTGCCTTATGTTGG
TTTTCTCGAACACATTGGCCGAATATTGGATCTTCAGTTGGAGGACAACAAATGGGCCTGCAATTGTG
ACTTATTGCAGTTAAAAACTTGGTTGGAGAACATGCCTCCACAGTCTATAATTGGTGATGTTGTCTGC
AACAGCCCTCCATTTTTTAAAGGAAGTATACTCAGTAGACTAAAGAAGGAATCTATTTGCCCTACTCC
ACCAGTGTATGAAGAACATGAGGATCCTTCAGGATCATTACATCTGGCAGCAACATCTTCAATAAATG
ATAGTCGCATGTCAACTAAGACCACGTCCATTCTAAAACTACCCACCAAAGCACCAGGTTTGATACCT
TATATTACAAAGCCATCCACTCAACTTCCAGGACCTTACTGCCCTATTCCTTGTAACTGCAAAGTCCT
ATCCCCATCAGGACTTCTAATACATTGTCAGGAGCGCAACATTGAAAGCTTATCAGATCTGAGACCTC
CTCCGCAAAATCCTAGAAAGCTCATTCTAGCGGGAAATATTATTCACAGTTTAATGAAGTCTGATCTA
GTGGAATATTTCACTTTGGAAATGCTTCACTTGGGAAACAATCGTATTGAAGTTCTTGAAGAAGGATC
GTTTATGAACCTAACGAGATTACAAAAACTCTATCTAAATGGTAACCACCTGACCAAATTAAGTAAAG
GCATGTTCCTTGGTCTCCATAATCTTGAATACTTATATCTTGAATACAATGCCATTAAGGAAATACTG
CCAGGAACCTTTAATCCAATGCCTAAACTTAAAGTCCTGTATTTAAATAACAACCTCCTCCAAGTTTT
ACCACCACATATTTTTTCAGGGGTTCCTCTAACTAAGGTAAATCTTAAAACAAACCAGTTTACCCATC
TACCTGTAAGTAATATTTTGGATGATCTTGATTTGCTAACCCAGATTGACCTTGAGGATAACCCCTGG
GACTGCTCCTGTGACCTGGTTGGACTGCAGCAATGGATACAAAAGTTAAGCAAGAACACAGTGACAGA
TGACATCCTCTGCACTTCCCCCGGGCATCTCGACAAAAAGGAATTGAAAGCCCTAAATAGTGAAATTC
TCTGTCCAGGTTTAGTAAATAACCCATCCATGCCAACACAGACTAGTTACCTTATGGTCACCACTCCT
GCAACAACAACAAATACGGCTGATACTATTTTACGATCTCTTACGGACGCTGTGCCACTGTCTGTTCT
AATATTGGGACTTCTGATTATGTTCATCACTATTGTTTTCTGTGCTGCAGGGATAGTGGTTCTTGTTC
TTCACCGCAGGAGAAGATACAAAAAGAAACAAGTAGATGAGCAAATGAGAGACAACAGTCCTGTGCAT
CTTCAGTACAGCATGTATGGCCATAAAACCACTCATCACACTACTGAAAGACCCTCTGCCTCACTCTA
TGAACAGCACATGGTGAGCCCCATGGTTCATGTCTATAGAAGTCCATCCTTTGGTCCAAAGCATCTGG
AAGAGGAAGAAGAGAGGAATGAGAAAGAAGGAAGTGATGCAAAACATCTCCAAAGAAGTCTTTTGGAA
CAGGAAAATCATTCACCACTCACAGGGTCAAATATGAAATACAAAACCACGAACCAATCAACAGAATT
TTTATCCTTCCAAGATGCCAGCTCATTGTACAGAAACATTTTAGAAAAAGAAAGGGAACTTCAGCAAC
TGGGAATCACAGAATACCTAAGGAAAAACATTGCTCAGCTCCAGCCTGATATGGAGGCACATTATCCT
GGAGCCCACGAAGAGCTGAAGTTAATGGAAACATTAATGTACTCACGTCCAAGGAAGGTATTAGTGGA
ACAGACAAAAAATGAGTATTTTGAACTTAAAGCTAATTTACATGCTGAACCTGACTATTTAGAAGTCC
TGGAGCAGCAAACATAG

| | | |
|---|---|---|
| NOV25c, CG55033-01 | | |
| Protein Sequence | SEQ ID NO: 570 798 aa | MW at 90352.1kD |

MLINCEAKGIKMVSEISVPPSRPFQLSLLNNGLTMLHTNDFSGLTNAISIHLGFNNIADIEIGAFNGL
GLLKQLHINHNSLEILKEDTFHGLENLEFLQADNNFITVIEPSAFSKLNRLKVLILNDNAIESLPPNI
FRFVPLTHLDLRGNQLQTLPYVGFLEHIGRILDLQLEDNKWACNCDLLQLKTWLENMPPQSIIGDVVC
NSPPFFKGSILSRLKKESICPTPPVYEEHEDPSGSLHLAATSSINDSRNSTKTTSILKLPTKAPGLIP
YITKPSTQLPGPYCPIPCNCKVLSPSGLLIHCQERNIESLSDLRPPPQNPRKLILAGNIIHSLMKSDL
VEYFTLEMLHLGNNRIEVLEEGSFMNLTRLQKLYLNGNHLTKLSKGMFLGLHNLEYLYLEYNAIKEIL
PGTFNPMPKLKVLYLNNNLLQVLPPHIFSGVPLTKVNLKTNQFTHLPVSNILDDLDLLTQIDLEDNPW
DCSCDLVGLQQWIQKLSKNTVTDDILCTSPGHLDKKELKALNSEILCPGLVNNPSMPTQTSYLMVTTP
ATTTNTADTILRSLTDAVPLSVLILGLLIMFITIVFCAAGIVVLVLHRRRRYKKKQVDEQMRDNSPVH
LQYSMYGHKTTHHTTERPSASLYEQHMVSPMVHVYRSPSFGPKHLEEEEERNEKEGSDAKHLQRSLLE
QENHSPLTGSNMKYKTTNQSTEFLSFQDASSLYRNILEKERELQQLGITEYLRKNIAQLQPDMEAHYP
GAHEELKLMETLMYSRPRKVLVEQTKNEYFELKANLEAEPDYLEVLEQQT

| | | |
|---|---|---|
| NOV25d, 237376826 | SEQ ID NO: 571 | 990 bp |
| DNA Sequence | ORF Start: at 1 | ORF Stop: end of sequence |

CGCGGTACCCCCTGGGACTGCTCCTGTGACCTGGTTGGACTGCAGCAATGGATACAAAAGTTAAGCAA
GAACACAGTGACAGATGACATCCTCTGCACTTCCCCCGGGCATCTCGACAAAAAGGAATTGAAAGCCC
TAAATAGTGAAATTCTCTGTCCAGGTTTAGTAAATAACCCATCCATGCCAACACAGACTAGTTACCTT
ATGGTCACCACTCCTGCAACAACAACAAATACGGCTGATACTATTTTACGATCTCTTACGGACGCTGT
GCCACTGTCTGTTCTAATATTGGGACTTCTGATTATGTTCATCACTATTGTTTTCTGTGCTGCAGGGA
TAGTGGTTCTTGTTCTTCACCGCAGGAGAAGATACAAAAAGAAACAAGTAGATGAGCAAATGAGAGAC
AACAGTCCTGTGCATCTTCAGTACAGCATGTATGGCCATAAAACCACTCATCACACTACTGAAAGACC
CTCTGCCTCACTCTATGAACAGCACATGGTGAGCCCCATGGTTCATGTCTATAGAAGTCCATCCTTTG
AGAAGTCTTTTGGAACAGGAAAATCATTCACCACTCACAGGGTCAAATATGAAATACAAAACCACGAA
CCAATCAACAGAATTTTTATCCTTCCAAGATGCCAGCTCATTGTACAGAAACATTTTAGAAAAAGAAA
GGGAACTTCAGCAACTGGGAATCACAGAATACCTAAGGAAAAACATTGCTCAGCTCCAGCCTGATATG
GAGGCACATTATCCTGGAGCCCACGAAGAGCTGAAGTTAATGGAAACATTAATGTACTCACGTCCAAG
GAAGGTATTAGTGGAACAGACAAAAAATGAGTATTTTGAACTTAAAGCTAATTTACATGCTGAACCTG
ACTATTTAGAAGTCCTGGAGCAGCAAACACTCGAGGCG

| | | | |
|---|---|---|---|
| NOV25d, 237376826 | | | |
| Protein Sequence | SEQ ID NO: 572 | 330 aa | MW at 37853.8kD |

RGTPWDCSCDLVGLQQWIQKLSKNTVTDDILCTSPGHLDKKELKALNSEILCPGLVNNPSMPTQTSYL
MVTTPATTTNTADTILRSLTDAVPLSVLILGLLIMFITIVFCAAGIVVLVLHRRRRYKKKQVDEQMRD
NSPVHLQYSMYGHKTTHHTTERPSASLYEQHMVSPMVHVYRSPSFGPKHLEEEEERNEKEGSDAKHLQ
EAHYPGAHEELKLMETLMYSRPRKVLVEQTKNEYFELKANLHAEPDYLEVLEQQTLEA

| | | |
|---|---|---|
| NOV25e, 310658403 | SEQ ID NO: 573 | 1648 bp |
| DNA Sequence | ORF Start: at 2 | ORF Stop: end of sequence |

CACCTCGCGAACCATGCTAATAAATTGTGAAGCAAAAGGTATCAAGATGGTATCTGAAATAAGTGTGC
CACCATCACGACCTTTCCAACTAAGCTTATTAAATAACGGCTTGACGATGCTTCACACAAATGACTTT
TCTGGGCTTACCAATGCTATTTCAATACACCTTGGATTTAACAATATTGCAGATATTGAGATAGGTGC
ATTTAATGGCCTTGGCCTCCTGAAACAACTTCATATCAATCACAATTCTTTAGAAATTCTTAAAGAGG
ATACTTTCCATGGACTGGAAAACCTGGAATTCCTGCAAGCAGATAACAATTTTATCACAGTGATTGAA
CCAAGTGCCTTTAGCAAGCTCAACAGACTCAAAGTGTTAATTTTAAATGACAATGCTATTGAGAGTCT
TCCTCCAAACATCTTCCGATTTGTTCCTTTAACCCATCTAGATCTTCGTGGAAATCAATTACAAACAT
TGCCTTATGTTGGTTTTCTCGAACACATTGGCCGAATATTGGATCTTCAGTTGGAGGACAACAAATGG
GCCTGCAATTGTGACTTATTGCAGTTAAAAACTTGGTTGGAGAACATGCCTCCACAGTCTATAATTGG
TGATGTTGTCTGCAACAGCCCTCCATTTTTTAAAGGAAGTATACTCAGTAGACTAAAGAAGGAATCTA
TTTGCCCTACTCCACCAGTGTATGAAGAACATGAGGATCCTTCAGGATCATTACATCTGGCAGCAACA
TCTTCAATAAATGATAGTCGCATGTCAACTAAGACCACGTCCATTCTAAAACTACCCACCAAAGCACC
AGGTTTGATACCTTATATTACAAAGCCATCCACTCAACTTCCAGGACCTTACTGCCCTATTCCTTGTA
ACTGCAAAGTCCTATCCCCATCAGGACTTCTAATACATTGTCAGGAGCGCAACATTGAAAGCTTATCA
GATCTGAGACCTCCTCCGCAAAATCCTAGAAAGCTCATTCTAGCGGAAAATATTATTCACAGTTTAAT
GAAGTCTGATCTAGTGGAATATTTCACTTTGGAAATGCTTCACTTGGGAAACAATCGTATTGAAGTTC
TTGAAGAAGGATCGTTTATGAACCTAACGAGATTACAAAAACTCTATCTAAATGGTAACCACCTGACC
AAATTAAGTAAAGGCATGTTCCTTGGTCTCCATAATCTTGAATACTTATATCTTGAATACAATGCCAT
TAAGGAAATACTGCCAGGAACCTTTAATCCAATGCCTAAACTTAAAGTCCTGTATTTAAATAACAACC
TCCTCCAAGTTTTACCACCACATATTTTTTCAGGGGTTCCTCTAACTAAGGTAAATCTTAAAACAAAC
CAGTTTACCCATCTACCTGTAAGTAATATTTTGGATGATCTTGATTTACTAACCCAGATTGACCTTGA
GGATAACCCCTGGGACTGCTCCTGTGACCTGGTTGGACTGCAGCAATGGATACAAAAGTTAAGCAAGA
ACACAGTGACAGATGACATCCTCTGCACTTCCCCCGGGCATCTCGACAAAAAGGAATTGAAAGCCCTA
AATAGTGAAATTCTCTGTCCAGGTTTAGTAAATAACCCATCCATGCCAACACAGACTAGTTACCTTAT
GGTCACCGTCGACGGC

| | | | |
|---|---|---|---|
| NOV25e, 310658403 | | | |
| Protein Sequence | SEQ ID NO: 574 | 549 aa | MW at 61376.4kD |

TSRTMLINCEAKGIKMVSEISVPPSRPFQLSLLNNGLTMLHTNDFSGLTNAISIHLGFNNIADIEIGA
FNGLGLLKQLHINHNSLEILKEDTFHGLENLEFLQADNNFITVIEPSAFSKLNRLKVLILNDNAIESL
PPNIFRFVPLTHLDLRGNQLQTLPYVGFLEHIGRILDLQLEDNKWACNCDLLQLKTWLENMPPQSIIG
DVVCNSPPFFKGSILSRLKKESICPTPPVYEEHEDPSGSLHLAATSSINDSRMSTKTTSILKLPTKAP
GLIPYITKPSTQLPGPYCPIPCNCKVLSPSGLLIHCQERNIESLSDLRPPPQNPRKLILAGNIIHSLM
KSDLVEYFTLEMLHLGNNRIEVLEEGSFMNLTRLQKLYLNGNHLTKLSKGMFLGLHNLEYLYLEYNAI
KEILPGTFNPMPKLKVLYLNNNLLQVLPPHIFSGVPLTKVNLKTNQFTHLPVSNILDDLDLLTQIDLE
DNPWDCSCDLVGLQQWIQKLSKNTVTDDILCTSPGHLDKKELKALNSEILCPGLVNNPSMPTQTSYLM
VTVDG

| | | |
|---|---|---|
| NOV25f, 317980876 | SEQ ID NO: 575 | 1887 bp |
| DNA Sequence | ORF Start: at 1 | ORF Stop: end of sequence |

ACAGTCTATAATTGGTGATGTTGTCTGCAACAGCCCTCCATTTTTTAAAGGAAGTATACTCAGTAGAC
TAAAGAAGGAATCTATTTGCCCTACTCCACCAGTGTATGAAGAACATGAGGATCCTTCAGGATCATTA
TAAAGAAGGAATCTATTTGCCCTACTCCACCAGTGTATGAAGAACATGAGGATCCTTCAGGATCATTA
ACCCACCAAAGCACCAGGTTTGATACCTTATATTACAAAGCCATCCACTCAACTTCCAGGACCTTACT
GCCCTATTCCTTGTAACTGCAAAGTCCTATCCCCATCAGGACTTCTAATACATTGTCAGGAGCGCAAC
ATTGAAAGCTTATCAGATCTGAGACCTCCTCCGCAAAATCCTAGAAAGCTCATTCTAGCGGGAAATAT
TATTCACAGTTTAATGAAGTCTGATCTAGTGGAATATTTCACTTTGGAAATGCTTCACTTGGGAAACA
GGTAACCACCTGACCAAATTAAGTAAAGGCATGTTCCTTGGTCTCCATAATCTTGAATACTTATATCT
ATTTAAATAACAACCTCCTCCAAGTTTTACCACCACATATTTTTTCAGGGGTTCCTCTAACTAAGGTA
CCAGATTGACCTTGAGGATAACCCCTGGGACTGCTCCTGTGACCTGGTTGGACTGCAGCAATGGATAC
AAAAGTTAAGCAAGAACACAGTGACAGATGACATCCTCTGCACTTCCCCCGGGCATCTCGACAAAAAG
GAATTGAAAGCCCTAAATAGTGAAATTCTCTGTCCAGGTTTAGTAAATAACCCATCCATGCCAACACA
GACTAGTTACCTTATGGTCACCACTCCTGCAACAACAACAAATACGGCTGATACTATTTTACGATCTC
TTACGGACGCTGTGCCACTGTCTGTTCTAATATTGGGACTTCTGATTATGTTCATCACTATTGTTTTC
TGTGCTGCAGGGATAGTGGTTCTTGTTCTTCACCGCAGGAGAAGATACAAAAAGAAACAAGTAGATGA
GCAAATGAGAGACAACAGTCCTGTGCATCTTCAGTACAGCATGTATGGCCATAAAACCACTCATCACA
AAAACATCTCCAAAGAAGTCTTTTGGAACAGGAAAATCATTCACCACTCACAGGGTCAAATATGAAAT
ACAAAACCACGAACCAATCAACAGAATTTTTATCCTTCCAAGATGCCAGCTCATTGTACAGAAACATT
TTAGAAAAAGAAAGGGAACTTCAGCAACTGGGAATCACAGAATACCTAAGGAAAAACATTGCTCAGCT
CCAGCCTGATATGGAGGCACATTATCCTGGAGCCCACGAAGAGCTGAAGTTAATGGAAACATTAATGT
ACTCACGTCCAAGGAAGGTATTAGTGGAACAGACAAAAAATGAGTATTTTGAACTTAAAGCTAATTTA
CATGCTGAACCTGACTATTTAGAAGTCCTGGAGCAGCAAACACTCGAGGCG

| | | | |
|---|---|---|---|
| NOV25f, 317980876 | | | |
| Protein Sequence | SEQ ID NO: 576 | 629 aa | MW at 71406.5kD |

RGTKWACNCDLLQLKTWLENMPPQSIIGDVVCNSPPFFKGSILSRLKKESICPTPPVYEEHEDPSGSL
HLAATSSINDSRMSTKTTSILKLPTKAPGLIPYITKPSTQLPGPYCPIPCNCKVLSPSGLLIHCQERN
IESLSDLRPPPQNPRKLILAGNIIHSLMKSDLVEYFTLEMLHLGNNRIEVLEEGSFMNLTRLQKLYLN
GNHLTKLSKGMFLGLHNLEYLYLEYNAIKEILPGTFNPMPKLKVLYLNNNLLQVLPPHIFSGVPLTKV
NLKTNQFTHLPVSNILDDLDLLTQIDLEDNPWDCSCDLVGLQQWIQKLSKNTVTDDILCTSPGHLDKK
ELKALNSEILCPGLVNNPSMPTQTSYLMVTTPATTTNTADTILRSLTDAVPLSVLILGLLIMFITIVF
CAAGIVVLVLHRRRRYKKKQVDEQMRDNSPVHLQYSMYGHKTTHHTTERPSASLYEQHMVSPMVHVYR
SPSFGPKHLEEEEERNEKEGSDAKILQRSLLEQENHSPLTGSNNKYKTTNQSTEFLSFQDASSLYRNI
LEKERELQQLGITEYLRKNIAQLQPDMEAHYPGAHEELKLMETLMYSRPRKVLVEQTKNEYFELKANL
HAEPDYLEVLEQQTLEA

| | | |
|---|---|---|
| NOV25g, 318018151 | SEQ ID NO: 577 | 1887 bp |
| DNA Sequence | ORF Start: at 1 | ORF Stop: end of sequence |

CGCGGTACCAAATGGGCCTGCAATTGTGACTTATTGCAGTTAAAAACTTGGTTGGAGAACATGCCCCC
ACAGTCTATAATTGGTGATGTTGTCTGCAACAGCCCTCCATTTTTTAAAGGAAGTATACTCAGTAGAC
TAAAGAAGGAATCTATTTGCCCTACTCCACCAGTGTATGAAGAACATGAGGATCCTTCAGGATCATTA
CATCTGGCAGCAACATCTTCAATAAATGATAGTCGCATGTCAACTAAGACCACGTCCATTCTAAAACT
ACCCACCAAAGCACCAGGTTTGATACCTTATATTACAAAGCCATCCACTCAACTTCCAGGACCTTACT
GCCCTATTCCTTGTAACTGCAAAGTCCTATCCCCATCAGGACTTCTAATACATTGTCAGGAGCGCAAC
ATTGAAAGCTTATCAGATCTGAGAGCCTCCTCCGCAAAATCCTAGAAGCTCATTCTAGCGGGAAATAT
TATTCACAGTTTAATGAAGTCTGATCTAGTGGAATATTTCACTTTGGAAATGCTTCACTTGGGAAACA
ATCGTATTGAAGTTCTTGAAGAAGGATCGTTTATGAACCTAACGAGATTACAAAAACTCTATCTAAAT
GGTAACCACCTGACCAAATTAAGTAAAGGCATGTTCCTTGGTCTCCATAATCTTGAATACTTATATCT
TGAATACAATGCCATTAAGGAAATACTGCCAGGAACCTTTAATCCAATGCCTAAACTTAAAGTCCTGT
ATTTAAATAACAACCTCCTCCAAGTTTTACCACCACATATTTTTTCAGGGGTTCCTCTAACTAAGGTA
AATCTTAAAACAAACCAGTTTACCCATCTACCTGTAAGTAATATTTTGGATGATCTTGATTTACTAAC
CCAGATTGACCTTGAGGATAACCCCTGGGACTGCTCCTGTGACCTGGTTGGACTGCAGCAATGGATAC
AAAAGTTAAGCAAGAACACAGTGACAGATGACATCCTCTGCACTTCCCCCGGGCATCTCGACAAAAAG
GAATTGAAAGCCCTAAATAGTGAAATTCTCTGTCCAGGTTTAGTAAATAACCCATCCATGCCAACACA
GACTAGTTACCTTATGGTCACCACTCCTGCAACAACAACAAATACGGCTGATACTATTTTACGATCTC
TGTGCTGCAGGGATAGTGGTTCTTGTTCTTCACCGCAGGAGAAGATACAAAAAGAAACAAGTAGATGA
GCAAATGAGAGACAACAGTCCTGTGCATCTTCAGTACAGCATGTATGGCCATAAAACCACTCATCACA
CTACTGAAAGACCCTCTGCCTCACTCTATGAACAGCACATGGTGAGCCCCATGGTTCATGTCTATAGA
AGTCCATCCTTTGGTCCAAAGCATCTGGAAGAGGAAGAAGAGAGGAATGAGAAAGAAGGAAGTGATGC
AAAACATCTCCAAAGAAGTCTTTTGGAACAGGAAAATCATTCACCACTTACAGGGTCAAATATGAAAT
ACAAAACCACGAACCAATCAACAGAATTTTTATCCTTCCAAGATGCCAGCTCATTGTACAGAAACATT
TTAGAAAAAGAAAGGGAACTTCAGCAACTGGGAATCACAGAATACCTAAGGAAAAACATTGCTCAGCT
CCAGCCTGATATGGAGGCACATTATCCTGGAGCCCACGAAGAGCTGAAGTTAATGGAAACATTAATGT
ACTCACGTCCAAGGAAGGTATTAGTGGAACAGACAAAAAATGAGTATTTTGAACTTAAAGCTAATTTA
CATGCTGAACCTGACTATTTAGAAGTCCTGGAGCAGCAAACACTCGAGGCG

| | | | |
|---|---|---|---|
| NOV25g, 318018151 | | | |
| Protein Sequence | SEQ ID NO: 578 | 629 aa | MW at 71406.5kD |

RGTKWACNCDLLQLKTWLENMPPQSIIGDVVCNSPPFFKGSILSRLKKESICPTPPVYEEHEDPSGSL
HLAATSSINDSRMSTKTTSILKLPTKAPGLIPYITKPSTQLPGPYCPIPCNCKVLSPSGLLIHCQERN
IESLSDLRPPPQNPRKLILAGNIIHSLMKSDLVEYFTLEMLHLGNNRIEVLEEGSFMNLTRLQKLYLN
GNHLTKLSKGMFLGLHNLEYLYLEYNAIKEILPGTFNPMPKLKVLYLNNNLLQVLPPHIFSGVPLTKV
NLKTNQFTHLPVSNILDDLDLLTQIDLEDNPWDCSCDLVGLQQWIQKLSKNTVTDDILCTSPGHLDKK
ELKALNSEILCPGLVNNPSMPTQPSYLMVTTPATTTNTADTILRSLTDAVPLSVLILGLLIMFITVIF
CAAGIVVLVLHRRRRYKKKQVDEQMRDNSPVHLQYSMYGHKTTHHTTERPSASLYEQHMVSPMVHVYR
SPSFGPKHLEEEEERNEKEGSDAKHLQRSLLEQENHSPLTGSNMKYKTTNQSTEFLSFQDASSLYRNI
LEKERELQQLGITEYLRKNIAQLQPDMEAHYPGAHEELKLMETLMYSRPRKVLVEQTKNEYFELKANL
HAEPDYLEXTLEQQTLEA

| | | |
|---|---|---|
| NOV25h, 318176316 | SEQ ID NO: 579 | 2433 bp |
| DNA Sequence | ORF Start: at 1 | ORF Stop: end of sequence |

CGCGGTACCACCATGCTAATAAATTGTGAAGCAAAAGGTATCAAGATGGTATCTGAAATAAGTGTGCC
ACCATCACGACCTTTCCAACTAAGCTTATTAAATAACGGCTTGACGATGCTTCACACAAATGACTTTT
CTGGGCTTACCAATGCTATTTCAATACACCTTGGATTTAACAATATTGCAGATATTGAGATAGGTGCA
TTTAATGGCCTTGGCCTCCTGAAACAACTTCATATCAATCACAATTCTTTAGAAATTCTTAAAGAGGA
CAAGTGCCTTTAGCAAGCTCAACAGACTCAAAGTGTTAATTTTAAATGACAATGCTATTGAGAGTCTT
CCTCCAAACATCTTCCGATTTGTTCCTTTAACCCATCTAGATCTTCGTGGAAATCAATTACAAACATT
GCCTTATGTTGGTTTTCTCGAACACATTGGCCGAATATTGGATCTTCAGTTGGAGGACAACAAATGGG
CCTGCAATTGTGACTTATTGCAGTTAAAAACTTGGTTGGAGAACATGCCTCCACAGTCTATAATTGGT
CTTCAATAAATGATAGTCGCATGTCAACTAAGACCACGTCCATTCTAAAACTACCCACCAAAGCACCA
CCTGCAAAGTCCTATCCCCATCAGGACTTCTAATACATTGTCAGGAGCGCAACATTGAAGCTTATCAG
GATGTTGTCTGCAACAGCCCTCCATTTTTTAAAGGAAGTATACTCAGTAGACTAAAGAAGGAATCTAT
TTGCCCTACTCCACCAGTGTATGAAGAACATGAGGATCCTTCAGGATCATTACATCTGGCAGCAACAT
CTTCAATAAATGATAGTCGCATGTCAACTAAGACCACGTCCATTCTAAAACTACCCACCAAAGCACCA
GGTTTGATACCTTATATTACAAAGCCATCCACTCAACTTCCAGGACCTTACTGCCCTATTCCTTGTAA
CTGCAAAGTCCTATCCCCATCAGGACTTCTAATACATTGTCAGGAGCGCAACATTGAAAGCTTATCAG
ATCTGAGACCTCCTCCGCAAAATCCTAGAAAGCTCATTCTAGCGGGAAATATTATTCACAGTTTAATG
AAGTTTACCCATCTACCTGTAAGTAATATTTTGGATGATCTTGATTTACTACCCAGATTGACCTTGAG
TGAAGAAGGATCGTTTACGAACCTAACGAGATTACAAAAACTCTATCTAAATGGTAACCACCTGACCA
AATTAAGTAAAGGCATGTTCCTTGGTCTCCATAATCTTGAATACTTATATCTTGAATACAATGCCATT
AAGGAAATACTGCCAGGAACCTTTAATCCAATGCCTAAACTTAAAGTCCTGTATTTAAATAACAACCT
GTCACCACTCCTGCAACAACAACAAATACGGCTGATACTATTTTACGATCTCTTACGGACGCTGTGCC
ACTGTCTGTTCTAATATTGGGACTTCTGATTATGTTCATCACTATTGTTTTCTGTGCTGCAGGGATAG
TGGTTCTTGTTCTTCACCGCAGGAGAAGATACAAGAAGAAACAAGTAGATGAGCAAATGAGAGACAAC
AGTCCTGTGCATCTTCAGTACAGCATGTATGGCCATAAAACCACTCATCACACTACTGAAAGACCCTC
TGCCTCACTCTATGAACAGCACATGGTGAGCCCCATGGTTCATGTCTATAGAAGTCCATCCTTTGGTC
CAAAGCATCTGGAAGAGGAAGAAGAGAGGAATGAGAAAGAAGGAAGTGATGCAAAACATCTCCAAAGA
AGTCTTTTGGAACAGGAAAATCATTCACCACTCACAGGGTCAAATATGAAATACAAAACCACAGAACC
ATCAACAGAATTTTTATCCTTCCAAGATGCCAGCTCATTGTACAGAAACATTTTAGAAAAAGAAAGGG
AACTTCAGCAACTGGGAATCACAGAATACCTAAGGAAAAACATTGCTCAGCTCCAGCCTGATATGGAG
GCACATTATCCTGGAGCCCACGAAGAGCTGAAGTTAATGGAAACATTAATGTACTCACGTCCAAGGAA
GGTATTAGTGGAGCAGACAAAAAATGAGTATTTTGAACTTAAAGCTAATTTACATGCTGAACCTGACT
ATTTAGAAGTCCTGGAGCAGCAAACACTCGAGGCGAAGGGCGAATTCCAGCAC

| | | | |
|---|---|---|---|
| NOV25h, 318176316 | | | |
| Protein Sequence | SEQ ID NO: 580 | 811 aa | MW at 91777.6kD |

RGTTMLINCEAKGIKMVSIESVPPSRPFQLSLLNNGLTMLHTNDFSGLTNAISIHLGFNNIADIEIGA
FNGLGLLKQLHINHNSLEILKEDTFHGLENLEFLQADNNFITVIEPSAFSKLNRLKVLILNDNAIESL
PPNIFRFVPLTHLDLRGNQLQTLPYVGFLEHIGRILDLQLEDNKWACNCDLLQLKTWLENMPPQSIIG
DVVCNSPPFFKGSILSRLKKESICPTPPVYEEHEDPSGSLHLAATSSINDSRMSTKTTSILKLPTKAP
GLIPYITKPSTQLPGPYCPIPCNCKVLSPSGLLIHCQERNIESLSDLRPPPQNPRKLILAGNIIHSLM
KSDLVEYFTLEMLHLGNNRIEVLEEGSFTNLTRLQKLYLNGNHLTKLSKGMFLGLHNLEYLYLEYNAI
KEILPGTFNPMPKLKVLYLNNNLLQVLPPHIFSGVPLTKVNLKTNQFTHLPVSNILDDLDLLTQIDLE
DNPWDCSCDLVGLQQWIQKLSKNTVTDDILCTSPGHLDKKELKALNSEILCPGLVNNPSMPTQTSYTM
VTTPATTTNTADTILRSLTDAVPLSVTILGLLIMFITIVFCAAGIVVLVLHRRRRYKKKQVDEQMRDN
SPVHLQYSMYGHKTTHHTTERPSASLYEQHMVSPMVHVYRSPSFGPKHLEEEEERNEKEGSDAKHLQR
SLLEQENHSPLTGSNMKYKTTNQSTEFLSFQDASSLYRNILEKERELQQLGITEYLRKNIAQLQPDME
AHYPGAHEELKLMETLMYSRPRKVLVEQTKNEYFELKANLHAEPDYLEVLEQQTLEAKGEFQH

| | | |
|---|---|---|
| NOV25i, CG55033-02 | SEQ ID NO: 581 | 1668 bp |
| DNA Sequence | ORF Start: ATG at 1 | ORE Stop: at 1627 |

ATGCTAATAAATTGTGAAGCAAAAGGTATCAAGATGGTATCTGAAATAAGTGTGCCACCATCACGACC
TTTCCAACTAAGCTTATTAAATAACGGCTTGACGATGCTTCACACAAATGACTTTTCTGGGCTTACCA
ATGCTATTTCAATACACCTTGGATTTAACAATATTGCAGATATTGAGATAGGTGCATTTAATGGCCTT
GGCCTCCTGAAACAACTTCATATCAATCACAATTCTTTAGAAATTCTTAAAGAGGATACTTTCCATGG
ACTGGAAAACCTGGAATTCCTGCAAGCAGATAACAATTTTATCACAGTGATTGAACCAAGTGCCTTTA
GCAAGCTCAACAGACTCAAAGTGTTAATTTTAAATGACAATGCTATTGAGAGTCTTCCTCCAAACATC
TTCCGATTTGTTCCTTTAACCCATCTAGATCTTCGTGGAAATCAATTACAAACATTGCCTTATGTTGG
TTTTCTCGAACACATTGGCCGAATATTGGATCTTCAGTTGGAGGACAACAAATGGGCCTGCAATTGTG
ACTTATTGCAGTTAAAAACTTGGTTGGAGAACATGCCTCCACAGTCTATAATTGGTGATGTTGTCTGC
AACAGCCCTCCATTTTTTAAAGGAATATACTCAGTAGACTAAAGAAGGAATCTATTTGCCCATACTCC
ACCAGTGTATGAAGAACATGAGGATCCTTCAGGATCATTACATCTGGCAGCAACATCTTCAATAAATG
ATAGTCGCATGTCAACTAAGACCACGTCCATTCTAAAACTACCCACCAAAGCACCAGGTTTGATACCT
TATATTACAAAGCCATCCACTCAACTTCCAGGACCTTACTGCCCTATTCCTTGTAACTGCAAAGTCCT
ATCCCCATCAGGACTTCTAATACATTGTCAGGAGCGCAACATTGAAAGCTTATCAGATCTGAGACCTC
CTCCGCAAAATCCTAGAAAGCTCATTCTAGCGGGAAATATTATTCACAGTTTAATGAAGTCTGATCTA
GTGGAATATTTCACTTTGGAAATGCTTCACTTGGGAAACAATCGTATTGAAGTTCTTGAAGAAGGATC
GTTTATGAACCTAACGAGATTACAAAAACTCTATCTAAATGGTAACCACCTGACCAAATTAAGTAAAG
GCATGTTCCTTGGTCTCCATAATCTTGAATACTTATATCTTGAATACAATGCCATTAAGGAAATACTG
CCAGGAACCTTTAATCCAATGCCTAAACTTAAAGTCCTGTATTTAAATAACAACCTCCTCCAAGTTTT
ACCACCACATATTTTTTCAGGGGTTCCTCTAACTAAGGTAAATCTTAAAACAAACCAGTTTACCCATC
TACCTGTAAGTAATATTTTGGATGATCTTGATTTGCTAACCCAGATTGACCTTGAGGATAACCCCTGG
GACTGCTCCTGTGACCTGGTTGGACTGCAGCAATGGATACAAAAGTTAAGCAAGAACACAGTGACAGA
TGACATCCTCTGCACTTCCCCCGGGCATCTCGACAAAAAGGAATTGAAAGCCCTAAATAGTGAAATTC
TCTGTCCAGGTTTAGTAAATAACCCATCCATGCCAACACAGACTAGTTACCTTATGGTCACCACTCCT
GCAACAACAACAAATACGGCTGATACTATTTTACGA

| | | | |
|---|---|---|---|
| NOV25i, CG55033-02 | | | |
| Protein Sequence | SEQ ID NO: 582 | 542 aa | MW at 60659.6kD |

MLINCEAKGIKMVSEISVPPSRPFQLSLLNNGLTMLHTNDFSGLTNAISIHLGFNNIADIEIGAFNGL
GLLKQLHINHNSLEILKEDTFHGLENLEFLQADNNFITVIEPSAFSKLNRLKVLILNDNAIESLPPNI
FRFVPLTHLDLRGNQLQTLPYVGFLEHIGRILDLQLEDNKWACNCDLLQLKTWLENMPPQSIIGDVVC
NSPPFFKGSILSRLKKESICPTPPVYEEHEDPSGSLHLAATSSINDSRMSTKTTSILKLPTKAPGLIP
YITKPSTQLPGPYCPIPCNCKVLSPSGLLIHCQERNIESLSDLRPPPQNPRXLILAGNIIHSLMKSDL
VEYFTLEMLHLGNNRIEVLEEGSFMNLTRLQKLYLNGNHLTKLSKGMFLGLHNLEYLYLEYNAIKEIL
PGTFNPMPKLKVLYLNNNLLQVLPPHIFSGVPLTKVNLKTNQFTHLPVSNILDDLDLLTQIDLEDNPW
DCSCDLVGLQQWIQKLSKNTVTDDILCTSPGHLDKKELKALNSEILCPGLVNNPSMPTQTSYLMVT

| | | |
|---|---|---|
| NOV25j, CG55033-03 | SEQ ID NO: 583 | 1668 bp |
| DNA Sequence | ORF Start: ATG at 1 | ORF Stop: end of sequence |

ATGCTAATAAATTGTGAAGCAAAAGGTATCAAGATGGTATCTGAAATAAGTGTGCCACCATCACGACC
TTTCCAACTAAGCTTATTAAATAACGGCTTGACGATGCTTCACACAAATGACTTTTCTGGGCTTACCA
ATGCTATTTCAATACACCTTGGATTTAACAATATTGCAGATATTGAGATAGGTGCATTTAATGGCCTT
GGCCTCCTGAAACAACTTCATATCAATCACAATTCTTTAGAAATTCTTAAAGAGGATACTTTCCATGG
ACTGGAAAACCTGGAATTCCTGCAAGCAGATAACAATTTTATCACAGTGATTGAACCAAGTGCCTTTA
GCAAGCTCAACAGACTCAAAGTGTTAATTTTAAATGACAATGCTATTGAGAGTCTTCCTCCAAACATC
TTCCGATTTGTTCCTTTAACCCATCTAGATCTTCGTGGAAATCAATTACAAACATTGCCTTATGTTGG
TTTTCTCGAACACATTGGCCGAATATTGGATCTTCAGTTGGAGGACAACAAATGGGCCTGCAATTGTG
ACTTATTGCAGTTAAAAACTTGGTTGGAGAACATGCCTCCACAGTCTATAATTGGTGATGTTGTCTGC
AACAGCCCTCCATTTTTTAAAGGAAGTATACTCAGTAGACTAAAGAAGGAATCTATTTGCCCTACTCC
ACCAGTGTATGAAGAACATGAGGATCCTTCAGGATCATTACATCTGGCAGCAACATCTTCAATAAATG
ATAGTCGCATATCAACTAAGACCACGTCCATTCTAAAACTACCCACCAAGGCACCAGGTTTGATACCT
TATATTACAAAGCCATCCACTCAACTTCCAGGACCTTACTGCCCTATTCCTTGTAACTGCAAAGTCCT
ATCCCCATCAGGACTTCTAATACATTGTCAGGAGCGCAACATTGAAAGCTTATCAGATCTGAGACCTC
CTCCGCAAAATCCTAGAAAGCTCATTCTAGCGGGAAATATTATTCACAGTTTAATGAAGTCTGATCTA
GTGGAATATTTCACCTTGGAAATGCTTCACTTGGGAAACAATCGTATTGAAGTTCTTGAAGAAGGATC
GTTTATGAACCTAACGAGATTACAAAAACTCTATCTAAATGGTAACCACCTGACCAAATTAAGTAAAG
GCATGTTCCTTGGTCTCCATAATCTTGAATACTTATATCTTGAATACAATGCCATTAAGGAAATACTG
CCAGGAACCTTTAATCCAATGCCTAAACTTAAAGTCCTGTATTTAAATAACAACCTCCTCCAAGTTTT
TACCTGTAAGTAATATTTTGGATGATCTTGATTTACTAACCCAGATTGACCTTGAGGATAACCCCTGG
GACTGCTCCTGTGACCTGGTTGGACTGCAGCAATGGATACAAAAGTTAAGCAAGAACACAGTGACAGA
TCTGTCCAQGTTTAGTAAATAACCCATCCATGCCAACACAGACTAGTTACCTTATGGTCACCACTCCT
GCAACAACAACAAATACGGCTGATACTATTTTACGA

| | | | |
|---|---|---|---|
| NOV25j, | | | |
| CG55033-03 | | | |
| Sequence | SEQ ID NO: 584 | 556 aa | MW at 62099.2kD |

MLINCEAKGIKMVSEISVPPSRPFQLSLLNNGLTMLHTNDFSGLTNAISIHLGFNNIADIEIFAFNGL
GLLKQLHINHNSLEILKEDTFHGLENLEFLQADNNFITVIEPSAFSKLNPLKVLILNDNAIESLPPNI
FRFVPLTHLDLRGNQLQTLPYVGFLEHIGRILDLQLEDNKWACNCDLLQLKTWLENMPPQSIIGDVVC
NSPPFFKGSILSRLKKESICPTPPVYEEHEDPSGSLHLAATSSINDSRISTKTTSILKLPTKAPGLIP
YITKPSTQLPGPYCPIPCNCKVLSPSGLLIHCQERNIESLSDLRPPPQNPRKLILAGNIIHSLMKSDL
VEYFTLEMLHLGNNRIEVLEEGSFMNLTRLQKLYLNGNHLTKLSKGMFLGLHNLEYLYLEYNAIKEIL
PGTFNPMPKLKVLYLNNNLLQVLPPHIFSGVPLTKVNLKTNQFTHLPVSNILDDLDLLTQIDLEDNPW
DCSCDLVGLQQWIQKLSKNTVTDDILCTSPGHLDKKELKALNSEILCPGLVNNPSMPTQTSYLMVTTP
ATTTNTADTILR

| | | |
|---|---|---|
| NOV25k, CG5503306 | SEQ ID NO: 585 | 2539 bp |
| DNA Sequence | ORF Start: ATG at 5 | ORF Stop: TAG at 2528 |

CAACATGAAGCTGTGGATTCATCTCTTTTATTCATCTCTCCTTGCCTGTATATCTTTACACTCCCAAA
CTCCAGTGCTCTCATCCAGAGGCTCTTGTGATTCTCTTTGCAATTGTGAGGAAAAAGATGGCACAATG
CTAATAAATTGTGAAGCAAAAGGTATCAAGATGGTATCTGAAATAAGTGTGCCACCATCACGACCTTT
CCAACTAAGCTTATTAAATAACGGCTTGACGATGCTTCACACAAATGACTTTTCTGGGCTTACCAATG
CTATTTCAATACACCTTGGATTTAACAATATTGCAGATATTGAGATAGGTGCATTTAATGGCCTTGGC
CTCCTGAAACAACTTCATATCAATCACAATTCTTTAGAAATTCTTAAAGAGGATACTTTCCATGGACT
GGAAAACCTGGAATTCCTGCAAGCAGATAACAATTTTATCACAGTGATTGAACCAAGTGCCTTTAGCA
AGCTCAACAGACTCAAAGTGTTAATTTTAAATGACAATGCTATTGAGAGTCTTCCTCCAAACATCTTC
CGATTTGTTCCTTTAACCCATCTAGATCTTCGTGGAAATCAATTACAAACATTGCCTTATGTTGGTTT
TCTCGAACACATTGGCCGAATATTGGATCTTCAGTTGGAGGACAACAAATGGGCCTGCAATTGTGACT
TATTGCAGTTAAAAACTTGGTTGGAGAACATGCCTCCACAGTCTATAATTGGTGATGTTGTCTGCAAC
AGCCCTCCATTTTTTAAAGGAAGTATACTCAGTAGACTAAAGAAGGAATCTATTTGCCCTACTCCACC
AGTGTATGAAGAACATGAGGATCCTTCAGGATCATTACATCTGGCAGCAACATCTTCAATAAATGATA
GTCGCATGTCAACTAAGACCACGTCCATTCTAAAACTACCCACCAAAGCACCAGGTTTGATACCTTAT
ATTACAAAGCCATCCACTCAACTTCCAGGACCTTACTGCCCTATTCCTTGTAACTGCAAAGTCCTATC
CCCATCAGGACTTCTAATACATTGTCAGGAGCGCAACATTGAAAGCTTATCAGATCTGAGACCTCCTC
CGCAAAATCCTAGAAAGCTCATTCTAGCGGGAAATATTATTCACAGTTTAATGAAGTCTGATCTAGTG
GAATATTTCACTTTGGAAATGCTTCACTTGGGAAACAATCGTATTGAAGTTCTTGAAGAAGGATCGTT
TATGAACCTAACGAGATTACAAAAACTCTATCTAAATGGTAACCACCTGACCAAATTAAGTAAAGGCA
TGTTCCTTGGTCTCCATAATCTTGAATACTTATATCTTGAATACAATGCCATTAAGGAAATACTGCCA
GGAACCTTTAATCCAATGCCTAAACTTAAAGTCCTGTATTTAAATAACAACCTCCTCCAAGTTTTACC
ACCACATATTTTTTCAGGGGTTCCTCTAACTAAGGTAAATCTTAAAACAAACCAGTTTACCCATCTAC
CTGTAAGTAATATTTTGGATGATCTTGATTTACTAACCCAGATTGACCTTGAGGATAACCCCTGGGAC
TGCTCCTGTGACCTGGTTGGACTGCAGCAATGGATACAAAAGTTAAGCAAGAACACAGTGACAGATGA
CATCCTCTGCACTTCCCCCGGGCATCTCGACAAAAAGGAATTGAAAGCCCTAAATAGTGAAATTCTCT
GTCCAGGTTTAGTAAATAACCCATCCATGCCAACACAGACTAGTTACCTTATGGTCACCACTCCTGCA
ACAACAACAAATACGGCTGATACTATTTTACGATCTCTTACGGACGCTGTGCCACTGTCTGTTCTAAT
ATTGGGACTTCTGATTATGTTCATCACTATTGTTTTCTGTGCTGCAGGGATAGTGGTTCTTGTTCTTC
ACCGCAGGAGAAGATACAAAAAGAAACAAGTAGATGAGCAAATGAGAGACAACAGTCCTGTGCATCTT
CAGTACAGCATGTATGGCCATAAAACCACTCATCACACTACTGAAAGACCCTCTGCCTCACTCTATGA
ACAGCACATGGTGAGCCCCATGGTTCATGTCTATAGAAGTCCATCCTTTGGTCCAAAGCATCTGGAAG
AGGAAGAAGAGAGGAATGAGAAAGAAGGAAGTGATGCAAAACATCTCCAAAGAAGTCTTTTGGAACAG
GAAAATCATTCACCACTCACAGGGTCAAATATGAAATACAAAACCACGAACCAATCAACAGAATTTTT
ATCCTTCCAAGATGCCAGCTCATTGTACAGAAACATTTTAGAAAAAGAAAGGGAACTTCAGCAACTGG
GAATCACAGAATACCTAAGGAAAAACATTGCTCAGCTCCAGCCTGATATGGAGGCACATTATCCTGGA
GACAAAAAATGAGTATTTTGAACTTAAAGCTAATTTACATGCTGAACCTGACTATTTAGAAGTCCTGG
AGCAGCAAACATAGATGGAGAGT

| | | | |
|---|---|---|---|
| NOV25k, CG55033-06 | | | |
| Protein Sequence | SEQ ID NO: 586 | 841 aa | MW at 95108.5kD |

MKLWIHLFYSSLLACISLHSQTPVLSSRGSCDSLCNCEEKDGTMLINCEAKGIKMVSEISVPPSRPFQ
LSLLNNGLTMLHTNDFSGLTNAISIHLGFNNIADIEIGAFNGLGLLKQLHINHNSLEILKEDTFHGLE
NLEFLQADNNFITVIEPSAFSKLNRLKVLILNDNAIESLPPNIFRFVPLTHLDLRGNQLQTLPYVGFL
EHIGRILDLQLEDNKWACNCDLLQLKTWLENMPPQSIIGDVVCNSPPFFKGSILSRLKKESICPTPPV
YEEHEDPSGSLHLAATSSINDSRMSTKTTSILKLPTKAPGLIPYITKPSTQLPGPYCPIPCNCKVLSP
SGLLIHCQERNIESLSDLRPPPQNPRKLILAGNIIHSLMKSDLVEYFTLEMLHLGNNRIEVLEEGSFM
NLTRLQKLYLNGNHLTKLSKGMFLGLHNLEYLYLEYNAIKEILPGTFNPMPKLKSLYLNNNLLQVLPP
HIFSGVPLTKVNLKTNQFTHLPVSNILDDLDLLTQIDLEDNPWDCSCDLVGLQQWIQKLSKNTVTDDI
LCTSPGHLDKKELKALNSEILCPGLVNNPSMPTQTSYLMVTTPATTTNTADTILRSLTDAVPLSVLIL
GLLIMFITIVFCAAGIVVLVLHRPRRYKKKQVDEQMRDNSPVHLQYSMYGHKTTHHTTERPSASLYEQ
HMVSPMVHVYRSPSFGPKHLEEEEERNEKEGSDAKHLQRSLLEQENHSPLTGSNMKYKTTNQSTEFLS
FQDASSLYRNILEKERELQQLGITEYLRKNIAQLQPDMEAHYPGAHEELKLMETLMYSRPRKVLVEQT
KNEYFELKANLHAEPDYLVELEQQT

| | | |
|---|---|---|
| NOV25l, CG55033-07 | SEQ ID NO: 587 | 1648 bp |
| DNA Sequence | ORF Start: ATG at 14 | ORF Stop: at 1640 |

CACCTCGCGAACCATGCTAATAAATTGTGAAGCAAAAGGTATCAAGATGGTATCTGAAATAAGTGTGC
CACCATCACGACCTTTCCAACTAAGCTTATTAAATAACGGCTTGACGATGCTTCACACAAATGACTTT
TCTGGGCTTACCAATGCTATTTCAATACACCTTGGATTTAACAATATTGCAGATATTGAGATAGGTGC
ATTTAATGGCCTTGGCCTCCTGAAACAACTTCATATCAATCACAATTCTTTAGAAATTCTTAAAGAGG
TCCTCCAAACATCTTCCGATTTGTTCCTTTAACCCATCTAGATCTTCGTGGAAATCAATTACAAACAT
TGCCTTATGTTGGTTTTCTCGAACACATTGGCCGAATATTGGATCTTCAGTTGGAGGACAACAAATGG
GCCTGCAATTGTGACTTATTGCAGTTAAAAACTTGGTTGGAGAACATGCCTCCACAGTCTATAATTGG
TGATGTTGTCTGCAACAGCCCTCCATTTTTTAAAGGAAGTATACTCAGTAGACTAAAGAAGGAATCTA
TTTGCCCTACTCCACCAGTGTATGAAGAACATGAGGATCCTTCAGGATCATTACATCTGGCAGCAACA
TCTTCAATAAATGATAGTCGCATGTCAACTAAGACCACGTCCATTCTAAAACTACCCACCAAAGCACC
AGGTTTGATACCTTATATTACAAAGCCATCCACTCAACTTCCAGGACCTTACTGCCCTATTCCTTGTA
ACTGCAAAGTCCTATCCCCATCAGGACTTCTAATACATTGTCAGGAGCGCAACATTGAAAGCTTATCA
GATCTGAGACCTCCTCCGCAAAATCCTAGAAAGCTCATTCTAGCGGGAAATATTATTCACAGTTTAAT
GAAGTCTGATCTAGTGGAATATTTCACTTTGGAAATGCTTCACTTGGGAAACAATCGTATTGAAGTTC
TTGAAGAAGGATCGTTTATGAACCTAACGAGATTACAAAAACTCTATCTAAATGGTAACCACCTGACC
AAATTAAGTAAAGGCATGTTCCTTGGTCTCCATAATCTTGAATACTTATATCTTGAATACAATGCCAT
TAAGGAAATACTGCCAGGAACCTTTAATCCAATGCCTAAACTTAAAGTCCTGTATTTAAATAACAACC
TCCTCCAAGTTTTACCACCACATATTTTTTCAGGGGTTCCTCTAACTAAGGTAAATCTTAAAACAAAC
CAGTTTACCCATCTACCTGTAAGTAATATTTTGGATGATCTTGATTTACTAACCCAGATTGACCTTGA
GGATAACCCCTGGGACTGCTCCTGTGACCTGGTTGGACTGCAGCAATGGATACAAAAGTTAAGCAAGA
ACACAGTGACAGATGACATCCTCTGCACTTCCCCCGGGCATCTCGACAAAAAGGAATTGAAAGCCCTA
AATAGTGAAATTCTCTGTCCAGGTTTAGTAAATAACCCATCCATGCCAACACAGACTAGTTACCTTAT
GGTCACCGTCGACGGC

| | | | |
|---|---|---|---|
| NOV25l, CG55033-07 | | | |
| Protein Sequence | SEQ ID NO: 588 | 542 aa | MW at 60659.6kD |

MLINCEAKGIKMVSEISVPPSRPFQLSLLNNGLTMLHTNDFSGLTNAISIHLGFNNIADIEIGAFNGL
GLLKQLHINHNSLEILKEDTFHGLENLEFLQADNNFITVIEPSAFSKLNRLKVLILNDNAIESLPPNI
FRFVPLTHLDLRGNQLQTLPYVGFLEHIGRILDLQLEDNKWACNCDLLQLKTWLENMPPQSIIGDVVC
NSPPFFKGSILSRLKKESICPTPPVYEEHEDPSGSLHLAATSSINDSRMSTKTTSILKLPTKAPGLIP
YITKPSTQLPGPYCPIPCNCKVLSPSGLLIHCQERNIESLSDLRPPPQNPRKLILAGNIIHSLMKSDL
VEYFTLEMLHLGNNRIEVLEEGSFMNLTRLQKLYLNGNHLTKLSKGMFLGLHNLEYLYLEYNAIKEIL
PGTFNPMPKLKTLYLNNNLLQVLPPHIFSGVPLTKVNLKTNQFTHLPVSNILDDLDLLTQIDLEDNPW
DCSCDLVGLQQWIQKLSKNTVTDDILCTSPGHLDKKELKALNSEILCPGLVNNPSMPTQTSYLMVT

| | | |
|---|---|---|
| NOV25m, CG55033-08 | SEQ ID NO: 589 | 643 bp |
| DNA Sequence | ORF Start: ATG at 9 | ORF Stop: at 1635 |

GCGGCCGCATGCTAATAAATTGTGAAGCAAAAGGTATCAAGATGGTATCTGAAATAAGTGTGCCACCA
TCACGACCTTTCCAACTAAGCTTATTAAATAACGGCTTGACGATGCTTCACACAAATGACTTTTCTGG
GCTTACCAATGCTATTTCAATACACCTTGGATTTAACAATATTGCAGATATTGAGATAGGTGCATTTA
ATGGCCTTGGCCTCCTGAAACAACTTCATATCAATCACAATTCTTTAGAAATTCTTAAAGAGGATACT
TTCCATGGACTGGAAAACCTGGAATTCCTGCAAGCAGATAACAATTTTATCACAGTGATTGAACCAAG
TGCCTTTAGCAAGCTCAACAGACTCAAAGTGTTAATTTTAAATGACAATGCTATTGAGAGTCTTCCTC
CAAACATCTTCCGATTTGTTCCTTTAACCCATCTAGATCTTCGTGGAAATCAATTACAAACATTGCCT
TATGTTGGTTTTCTCGAACACATTGGCCGAATATTGGATCTTCAGTTGGAGGACAACAAATGGGCCTG
CAATTGTGACTTATTGCAGTTAAAAACTTGGTTGGAGAACATGCCTCCACAGTCTATAATTGGTGATG
TTGTCTGCAACAGCCCTCCATTTTTTAAAGGAAGTATACTCAGTAGACTAAAGAAGGAATCTATTTGC
CCTACTCCACCAGTGTATGAAGAACATGAGGATCCTTCAGGATCATTACATCTGGCAGCAACATCTTC
AATAAATGATAGTCGCATGTCAACTAAGACCACGTCCATTCTAAAACTACCCACCAAAGCACCAGGTT
TGATACCTTATATTACAAAGCCATCCACTCAACTTCCAGGACCTTACTGCCCTATTCCTTGTAACTGC
AAAGTCCTATCCCCATCAGGACTTCTAATACATTGTCAGGAGCGCAACATTGAAAGCTTATCAGATCT
GAGACCTCCTCCGCAAAATCCTAGAAAGCTCATTCTAGCGGGAAATATTATTCACAGTTTAATGAAGT
CTGATCTAGTGGAATATTTCACTTTGGAAATGCTTCACTTGGGAAACAATCGTATTGAAGTTCTTGAA
GAAGGATCGTTTATGAACCTAACGAGATTACAAAAACTCTATCTAAATGGTAACCACCTGACCAAATT
AAGTAAAGGCATGTTCCTTGGTCTCCATAATCTTGAATACTTATATCTTGAATACAATGCCATTAAGG
CAAGTTTTACCACCACATATTTTTTCAGGGGTTCCTCTAACTAAGGTAAATCTTAAAACAAACCAGTT
TACCCATCTACCTGTAAGTAATATTTTGGATGATCTTGATTTACTAACCCAGATTGACCTTGAGGATA
ACCCCTGGGACTGCTCCTGTGACCTGGTTGGACTGCAGCAATGGATACAAAAGTTAAGCAAGAACACA
GTGACAGATGACATCCTCTGCACTTCCCCCGGGCATCTCGACAAAAGGAAATTGAAAGCCCTAAATAG
TGAAATTCTCTGTCCAGGTTTAGTAAATAACCCATCCATGCCAACACAGACTAGTTACCTTATGGTCA
CCCTCGAGGGC

| | | | |
|---|---|---|---|
| NOV25m, CG55033-08 | | | |
| Protein Sequence | SEQ ID NO: 590 | 542 aa | MW at 60659.6kD |

MLINCEAKGIKMVSEISVPPSRPFQLSLLNNGLTMLHTNDFSGLTNAISIHLGFNNIADIEIGAFNGL
GLLKQLHINHNSLEILKEDTFHGLENLEFLQADNNFITVIEPSAFSKLNRLKVLILNDNAIESLPPNI
FRFVPLTHLDLRGNQLQTLPYVGFLEHIGRILDLQLEDNKWACNCDLLQLKTWLENMPPQSIIGDVVC
NSPPFFKGSILSRLKKESICPTPPVYEEHEDPSGSLHLAATSSINDSRMSTKTTSILKLPTKAPGLIP
YITKPSTQLPGPYCPIPCNCKVLSPSGLLIHCQERNIESLSDLRPPPQNPRKLILAGNIIHSLMKSDL
VEYFTLEMLHLGNNRIEVLEEGSFMNLTRLQKLYLNGNHLTKLSKGMFLGLHNLEYLYLEYNAIKEIL
PGTFNPMPKLKVLYLNNNLLQVLPPHIFSGVPLTKVNLKTNQFTHLPVSNILDDLDLLTQIDLEDNPW
DCSCDLVGLQQWIQKLSKNTVTDDILCTSPGHLDKKELKALNSETLCPGLVNNPSMPTQTSYLMVT

| | | |
|---|---|---|
| NOV25n, CG55033-09 | SEQ ID NO: 591 | 2838 bp |
| DNA Sequence | ORF Start: ATG at 184 | ORF Stop: TAG at 2707 |

AACTTTATGAAGCTATGGGACTTGACAAAAAGTGATATTTGAGAAGAAAGTACGCAGTGGTTGGTGTT
TTCTTTTTTTTAATAAAGGAATTGAATTACTTTGAACACCTCTTCCAGCTGTGCATTACAGATAACGT
CAGGAAGAGTCTCTGCTTTACAGAATCGGATTTCATCACATGACAACATGAAGCTGTGGATTCATCTC
TTTTATTCATCTCTCCTTGCCTGTATATCTTTACACTCCCAAACTCCAGTGCTCTCATCCAGAGGCTC
TCAAGATGGTATCTGAAATAAGTGTGCCACCATCACGACCTTTCCAACTAAGCTTATTAAATAACGGC
TTGACGATGCTTCACACAAATGACTTTTCTGGGCTTACCAATGCTATTTCAATACACCTTGGATTTAA
CAATATTGCAGATATTGAGATAGGTGCATTTAATGGCCTTGGCCTCCTGAAACAACTTCATATCAATC
ACAATTCTTTAGAAATTCTTAAAGAGGATACTTTCCATGGACTGGAAAACCTGGAATTCCTGCAAGCA
GATAACAATTTTATCACAGTGATTGAACCAAGTGCCTTTAGCAAGCTCAACAGACTCAAAGTGTTAAT
TTTAAATGACAATGCTATTGAGAGTCTTCCTCCAAACATCTTCCGATTTGTTCCTTTAACCCATCTAG
ATCTTCGTGGAAATCAATTACAAACATTGCCTTATGTTGGTTTTCTCGAACACATTGGCCGAATATTG
GATCTTCAGTTGGAGGACAACAAATGGGCCTGCAATTGTGACTTATTGCAGTTAAAAACTTGGTTGGA
GAACATGCCTCCACAGTCTATAATTGGTGATGTTGTCTGCAACAGCCCTCCATTTTTTAAAGGAAGTA
TACTCAGTAGACTAAAGAAGGAATCTATTTGCCCTACTCCACCAGTGTATGAAGAACATGAGGATCCT
TCAGGATCATTACATCTGGCAGCAACATCTTCAATAAATGATAGTCGCATGTCAACTAAGACCACGTC
CATTCTAAAACTACCCACCAAAGCACCAGGTTTGATACCTTATATTACAAAGCCATCCACTCAACTTC
CAGGACCTTACTGCCCTATTCCTTGTAACTGCAAAGTCCTATCCCCATCAGGACTTCTAATACATTGT
CAGGAGCGCAACATTGAAAGCTTATCAGATCTGAGACCTCCTCCGCAAAATCCTAGAAAGCTCATTCT
AGCGGGAAATATTATTCACAGTTTAATGAAGTCTGATCTAGTGGAATATTTCACTTTGGAAATGCTTC
ACTTGGGAAACAATCGTATTGAAGTTCTTGAAGAAGGATCGTTTATGAACCTAACGAGATTACAAAAA
CTCTATCTAAATGGTAACCACCTGACCAAATTAAGTAAAGGCATGTTCCTTGGTCTCCATAATCTTGA
ATACTTATATCTTGAATACAATGCCATTAAGGAAATACTGCCAGGAACCTTTAATCCAATGCCTAAAC
TTAAAGTCCTGTATTTAAATAACAACCTCCTCCAAGTTTTACCACCACATATTTTTTCAGGGGTTCCT
CTAACTAAGGTAAATCTTAAAACAAACCAGTTTACCCATCTACCTGTAAGTAATATTTTGGATGATCT
TGATTTACTAACCCAGATTGACCTTGAGGATAACCCCTGGGACTGCTCCTGTGACCTGGTTGGACTGC
CTCGACAAAAAGGAATTGAAAGCCCTALATAGTGAAATTCTCTGTCCAGGTTTAGTAAATAACCCATC
CATGCCAACACAGACTAGTTACCTTATGGTCACCACTCCTGCAACAACAACAAATACGGCTGATACTA
TTTTACGATCTCTTACGGACGCTGTGCCACTGTCTGTTCTAATATTGGGACTTCTGATTATGTTCATC
ACTATTGTTTTCTGTGCTGCAGGGATAGTGGTTCTTGTTCTTCACCGCAGGAGAAGATACAAAAAGAA
ACAAGTAGATGAGCAAATGAGAGACAACAGTCCTGTGCATCTTCAGTACAGCATGTATGGCCATAAAA
CCACTCATCACACTACTGAAAGACCCTCTGCCTCACTCTATGAACAGCACATGGTGAGCCCCATGGTT
CATGTCTATAGAAGTCCATCCTTTGGTCCAAAGCATCTGGAAGAGGAAGAAGAGAGGAATGAGAAAGA
AGGAAGTGATGCAAAACATCTCCAAAGAAGTCTTTTGGAACAGGAAAATCATTCACCACTCACAGGGT
TACAGAAACATTTTAGAAAAAGAAAGGGAACTTCAGCAACTGGGAATCACAGAATACCTAAGGAAAAA
CATTGCTCAGCTCCAGCCTGATATGGAGGCACATTATCCTGGAGCCCACGAAGAGCTGAAGTTAATGG
AAACATTAATGTACTCACGTCCAAGGAAGGTATTAGTGGAACAGACAAAAAATGAGTATTTTGAACTT
AAAGCTAATTTACATGCTGAACCTGACTATTTAGAAGTCCTGGAGCAGCAAACATAGATGGAGAGTAT
GAGGGCTTTCGCAGAAATGCTGTGATTCTGTTTTAAGTCCATACCTTGTAAATTAGTGCCTTACGTGA
GTGTGTCATCCATCAGAACCTAAGCACAGCAGTAAACTATGGAGAAAAAA

| | | | |
|---|---|---|---|
| NOV25n, CG55033-09 | | | |
| Protein Sequence | SEQ ID NO: 592 | 841 aa | MW at 95108.5kD |

MKLWIHLFYSSLLACISLHSQTPVLSSRGSCDSLCNCEEKDGTMLINCEAKGIKMVSEISVPPSRPFQ
LSLLNNGLTMLHTNDFSGLTNAISIHLGFNNIADIEIGAFNGLGLLKQLHINHNSLEILKEDTFNGLE
NLEFLQADNNFITVIEPSAFSKLNRLKVLILNDNAIESLPPNIFRFVPLTHLDLRGNQLQTLPYVGFL
EHIGRILDLQLEDNKWACNCDLLQLKTWLENMPPQSIIGDVVCNSPPFFKGSILSRLKKESICPTPPV
YEEHEDPSGSLHLAATSSINDSRMSTKTTSILKLPTKAPGLIPYITKPSTQLPGPYCPIPCNCKVLSP
SGLLIHCQERNIESLSDLRPPPQNPRKLILAGNIIHSLMKSDLVEYFTLEMLHLGNNRIEVLEEGSFM
NLTRLQKLYLNGNHLTKLSKGMFLGLHNLEYLYLEYNAIKEILPGTFNPMPKLKVLYLNNNLLQVLPP
HIFSGVPLTKVNLKTNQFTHLPVSNILDDLDLLTQIDLEDNPWDCSCDLVGLQQWIQKLSKNTVTDDI
LCTSPGHLDKKELKALNSEILCPGLVNNPSMPTQTSYLMVTTPATTTNTADTILRSLTDAVPLSVLIL
GLLIMFITIVFCAAGIVVLVLHRRRRYKKKQVDEQMRDNSPVHLQYSMYGHKTTHHTTERPSASLYEQ
FQDASSLYRNILEKERELQQLGITEYLRKNIAQLQPDMEAHYPGAHEELKLMETLMYSRPRKVLVEQT
KNEYFELKANLHAEPDYLEVLEQQT

| | | |
|---|---|---|
| NOV25o, CG55033-10 | SEQ ID NO: 593 | 2531 bp |
| DNA Sequence | ORF Start: at 2 | ORF Stop: TAA at 2513 |

GGATTCTCTCTTTTATTCATCTCTCCTTGCCTGTATATCTTTACACTCCCAAACTCCAGTGCTCTCAT
CCAGAGGCTCTTGTGATTCTCTTTGCAATTGTGAGGAAAAAGATGGCACAATGCTAATAAATTGTGAA
GCAAAAGGTATCAAGATGGTATCTGAAATAAGTGTGCTACCATCACGACCTTTCCAACTAAGCTTATT
AAATAACGGCTTGACGATGCTTCACACAAATGACTTTTCTGGGCTTACCAATGCTATTTCAATACACC
TTGGATTTAACAATATTGCAGATATTGAGATAGGTGCATTTAATGGCCTTGGCCTCCTGAAACAACTT
CATATCAATCACAATTCTTTAGAAATTCTTAAAGAGGATACTTTCCATGGACTGGAAAACCTGGAATT
CCTGCAAGCAGATAACAATTTTATCACAGTGATTGAACCAAGTGCCTTTAGCAAGCTCAACAGACTCA
AAGTGTTAATTTTAAATGACAATGCTATTGAGAGTCTTCCTCCAAACATCTTCCGATTTGTTCCTTTA
ACCCATCTAGATCTTCGTGGAAATCAATTACAAACATTGCCTTATGTTGGTTTTCTCGAACACATTGG
CCGAATATTGGATCTTCAGTTGGAGGACAACAAATGGGCCTGCAATTGTGACTTATTGCAGTTAAAAA
CTTGGTTGGAGAACATGCCTCCACAGTCTATAATTGGTGATGTTGTCTGCAACAGCCCTCCATTTTTT
AAAGGAAGTATACTCAGTAGACTAAAGAAGGAATCTATTTGCCCTACTCCACCAGTGTATGAAGAACA
TGAGGATCCTTCAGGATCATTACATCTGGCAGCAACATCTTCAATAAATGATAGTCGCATGTCAACTA
AGACCACGTCCATTCTAAAACTACCCACCAAAGCACCAGGTTTGATACCTTATATTACAAAGCCATCC
ACTCAACTTCCAGGACCTTACTGCCCTATTCCTTGTAACTGCAAAGTCCTATCCCCATCAGGACTTCT
AATACATTGTCAGGAGCGCAACAATTGAAAGCTTATCAGATCTGAGACCTCCTCCGCAAATCCTAGAA
AGCTCATTCTAGCGGGAAATATTATTCACAGTTTAATGAAGTCTGATCTAGTGGAATATTTCACTTTG
GAATGCTTCACTTGGGAACAATCGTATTGAAGTTCTTGAAGAAGGATCGTTTATGAACCTAAAACGAG
ATTACAAAAACTCTATCTAAATGGTAACCACCTGACCAAATTAAGTAAAGGCATGTTCCTTGGTCTCC
ATAATCTTGAATACTTATATCTTGAATACAATGCCATTAAGGAAATACTGCCAGGAACCTTTAATCCA
ATGCCTAAACTTAAAGTCCTGTATTTAAATAACAACCTCCTCCAAGTTTTACCACCACATATTTTTTC
AGGGGTTCCTCTAACTAAGGTAAATCTTAAAACAAACCAGTTTACCCATCTACCTGTAAGTAATATTT
TGGATGATCTTGATTTGCTAACCCAGATTGACCTTGAGGATAACCCCTGGGACTGCTCCTGTGACCTG
GTTGGACTGCAGCAATGGATACAAAAGTTAAGCAAGAACACAGTGACAGATGACATCCTCTGCACTTC
CCCCGGGCATCTCGACAAAAAGGAATTGAAAGCCCTAAATAGTGAAATTCTCTGTCCAGGTTTAGTAA
ATAACCCATCCATGCCAACACAGACTAGTTACCTTATGGTCACCACTCCTGCAACAACAACAAATACG
GCTGATACTATTTTACGATCTCTTACGGACGCTGTGCCACTGTCTGTTCTAATATTGGGACTTCTGAT
TATGTTCATCACTATTGTTTTCTGTGCTGCAGGGATAGTGGTTCTTGTTCTTCACCGCAGGAGAAGAT
ACAAAAAGAAACAAGTAGATGAGCAAATGAGAGACAACAGTCCTGTGCATCTTCAGTACAGCATGTAT
GGCCATAAAACCACTCATCACACTACTGAAAGACCCTCTGCCTCACTCTATGAACAGCACATGGTGAG
CCCCATGGTTCATGTCTATAGAAGTCCATCCTTTGGTCCAAAGCATCTGGAAGAGGAAGAAGAGAGGA
ATGAGAAAGAAGGAAGTGATGCAAAACATCTCCAAAGAAGTCTTTTGGAACAGGAAAATCATTCACCA
CTCACAGGGTCAAATATGAAATACAAAACCACGAACCAATCAACAGAATTTTTATCCTTCCAAGATGC
CAGCTCATTGTACAGAAACATTTTAGAAAAAGAAAGGGAACTTCAGCAACTGGGAATCACAGAATACC
TAAGGAAAAAACATTGCTCAGCTCCAGCCTGATATGGAGGCACATTATCCTGGAGCCACGAAGAGCTG
AAGTTAATGGAACATTAATGTACTCACGTCCAAGGAAGGTATTAGTGGAACAGACAAAAAAATGAGTA
TTTTGAACTTAAAGCTAATTTACATGCTGAACCTGACTATTTAGAAGTCCTGGAGCAGCAAACATAAG
GGCGAATTCTGCTGT

| | | | |
|---|---|---|---|
| NOV25o, CG55033-10 | | | |
| Protein Sequence | SEQ ID NO: 594 | 837 aa | MW at 94517.6kD |

DSLFYSSLLACISLHSQTPVLSSRGSCDSLCNCEEKDGTMLINCEAKGIKMVSEISVLPSRPFQLSLL
NNGLTMLHTNDFSGLTNAISIHLGFNNIADIEIGAFNGLGLLKQLHINHNSLEILKEDTFHGLENLEF
LQADNNFITVIEPSAFSKLNRLKVLILNDNAIESLPPNIFRFVPLTHLDLRGNQLQTLPYVGFLEHIG
RILDLQLEDNKWACNCDLLQLKTWLENMPPQSIIGDVVCNSPPFFKGSILSRLKKESICPTPPVYEEH
EDPSGSLHLAATSSINDSRMSTKTTSILKLPTKAPGLIPYITKPSTQLPGPYCPIPCNCKVLSPSGLL
IHCQERNIESLSDLRPPPQNPRKLILAGNIIHSLMKSDLVEYFTLEMLHLGNNRIEVLEEGSFMNLTR
LQKLYLNGNHLTKLSKGMFLGLHNLEYLYLEYNAIKEILPGTFNPMPKLKVLYLNNNLLQVLPPHIFS
GVPLTKVNLKTNQFTHLPVSNILDDLDLLTQIDLEDNPWDCSCDLVGLQQWIQKLSKNTVTDDILCTS
PGHLDKKELKALNSEILCPGLVNNPSMPTQTSYLMVTTPATTTNTADTILRSLTDAVPLSVLILGLLI
MFITIVFCAAGIVVLVLHRRRRYKKKQVDEQMRDNSPVHLQYSMYGHKTTHHTTERPSASLYEQHMVS
PMVHVYRSPSFFGPKHLEEEEERNEKEGSDAKHLQRSLLEQENHSPLTGSNMYKTTNQSTEFLSFQDA
SSLYRNILEKERELQQLGITEYLRKNIAQLQPDMEAHYPGAHEELKLMETLMYSRPRKVLVEQTKNEY
FELKANLHAEPDYLEVLEQQT

| | | |
|---|---|---|
| NOV25p, CG55033-11 | SEQ ID NO: 595 | 1758 bp |
| DNA Sequence | ORF Start: at 10 | ORF Stop: at 1750 |

CGCGGATCCCAAACTCCAGTGCTCTCATCCAGAGGCTCTTGTGATTCTCTTTGCAATTGTGAGGAAAA
AGATGGCACAATGCTAATAAATTGTGAAGCAAAAGGTATCAAGATGGTATCTGAAATAAGTGTGCCAC
CATCACGACCTTTCCAACTAAGCTTATTAAATAACGGCTTGACGATGCTTCACACAAATGACTTTTCT
GGGTTACCAATGCTATTTCAATACACCTTGGATTTAACAATATTGCAGATATTGAGATAGGTAGCATT
TAATGGCCTTGGCCTCCTGAAACAACTTCATATCAATCACAATTCTTTAGAAATTCTTAAAGAGGATA
CTTTCCATGGACTGGAAAACCTGGAATTCCTGCAAGCAGATAACAATTTTATCACAGTGATTGAACCA
AGTGCCTTTAGCAAGCTCAACAGACTCAAAGTGTTAATTTTAAATGACAATGCTATTGAGAGTCTTCC
TCCAAACATCTTCCGATTTGTTCCTTTAACCCATCTAGATCTTCGTGGAAATCAATTACAAACATTGC
CTTATGTTGGTTTTCTCGAACACATTGGCCGAATATTGGATCTTCAGTTGGAGGACAACAAATGGGCC
TGCAATTGTGACTTATTGCAGTTAAAAACTTGGTTGGAGAACATGCCTCCACAGTCTATAATTGGTGA
TGTTGTCTGCAACAGCCCTCCATTTTTTAAAGGAAGTATACTCAGTAGACTAAGAAGGAATCTAATTT
GCCCTACTCCACCAGTGTATGAAGAACATGAGGATCCTTCAGGATCATTACATCTGGCAGCAACATCT
TCAATAAATGATAGTCGCATGTCAACTAAGACCACGTCCATTCTAAAACTACCCACCAAAGCACCAGG
TTTGATACCTTATATTACAAAGCCATCCACTCAACTTCCAGGACCTTACTGCCCTATTCCTTGTAACT
GCAAAGTCCTATCCCCATCAGGACTTCTAATACATTGTCAGGAGCGCAACATTGAAAGCTTATCAGAT
CTGAGACCTCCTCCGCAAAATCCTAGAAAGCTCATTCTAGCGGGAAATATTATTCACAGTTTAATGAA
GTCTGAGCTAGTGGAATATTTCACTTTGGAAATGCTTCACTTGGGAAACAATCGTATTGAAGTTCTTG
AAGAAGGATCGTTTATGAACCTAACGAGATTACAAAAACTCTATCTAAATGGTAACCACCTGACCAAA
TTAAGTAAAGGCATGTTCCTTGGTCTCCATAATCTTGAATACTTATATCTTGAATACAATGCCATTAA
GGAATACTGCCAGGAACCTTTAATCCAATGCCTAAACTTAAAGTCCTGTATTTAAATAAACAACCTCC
TCCAAGTTTTACCACCACATATTTTTTCAGGGGTTCCTCTAACTAAGGTALATCTTAAAACAAACCAG
TTTACCCATCTACCTGTAAGTAATATTTTGGATGATCTTGATTTACTAACCCAGATTGACCTTGAGGA
TAACCCCTGGGACTGCTCCTGTGACCTGGTTGGACTGCAGCAATGGATACAAAAGTTAAGCAAGAACA
CAGTGACAGATGACATCCTCTGCACTTCCCCCGGGCATCTCGACAAAAAGGAATTGAAAGCCCTAAAT
AGTGAAATTCTCTGTCCAGGTTTAGTAAATAACCCATCCATGCCAACACAGACTAGTTACCTTATGGT
CACCACTCCTGCAACAACAACAAATACGGCTGATACTATTTTACGATCTCTCGAGGCG

| | | | |
|---|---|---|---|
| NOV25p, CG55033-11 | | | |
| Protein Sequence | SEQ ID NO: 596 | 580 aa | MW at 64615.9kD |

QTPVLSSRGSCDSLCNCEEKDGTMLINCEAKGIKMVSEISVPPSRPFQLSLLNNGLTMLHTNDFSGLT
NAISIHLGFNNIADIEIGAFNGLGLLKQLHINHNSLEILKEDTFHGLENLEFLQADNNFITVIEPSAF
SKLNRLKVLILNDNAIESLPPNIFRFVPLTHLDLRGNQLQTLPYVGFLEHIGRILDLQLEDNKWACNC
DLLQLKTWLENMPPQSIIGDVVCNSPPFFKGSILSRLKKESICPTPPVYEEHEDPSGSLHLAATSSIN
DSRMSTKTTSILKLPTKAPGLIPYITKPSTQLPGPYCPIPCNCKVLSPSGLLIHCQERNIESLSDLRP
PPQNPRKLILAGNIIHSLMKSDLVEYFTLEMLHLGNNRIEVLEEGSFMNLTRLQKLYLNGNHLTKLSK
GMFLGLHNLEYLYLEYNAIKEILPGTFNPMPKLKVLYLNNNLLQVLPPHIFSGVPLTKVNLKTNQFTH
LPVSNILDDLDLLTQIDLEDNPWDCSCDLVGLQQWIQKLSKNTVTDDILCTSPGHLDKKELKALNSEI
LCPGLVNNPSMPTQTSYLMVTTPATTTNTADTILRS

| | | |
|---|---|---|
| NOV25q, CG55033-12 | SEQ ID NO: 597 | 1762 bp |
| DNA Sequence | ORF Start: at 14 | ORF Stop: at 1754 |

CACCTCGCGAACCCAAACTCCAGTGCTCTCATCCAGAGGCTCTTGTGATTCTCTTTGCAATTGTGAGG
AAAAAGATGGCACAATGCTAATAAATTGTGAAGCAAAAGGTATCAAGATGGTATCTGAAATAAGTGTG
CCACCATCACGACCTTTCCAACTAAGCTTATTAAATAACGGCTTGACGATGCTTCACACAAATGACTT
CATTTAATGGCCTTGGCCTCCTGAAACAACTTCATATCAATCACAATTCTTTAGAAATTCTTAAAGAG
GATACTTTCCATGGACTGGAAAACCTGGAATTCCTGCAAGCAGATAACAATTTTATCACAGTGATTGA
ACCAAGTGCCTTTAGCAAGCTCAACAGACTCAAAGTGTTAATTTTAAATGACAATGCTATTGAGAGTC
TTCCTCCAAACATCTTCCGATTTGTTCCTTTAACCCATCTAGATCTTCGTGGAAATCAATTACAAACA
TTGCCTTATGTTGGTTTTCTCGAACACATTGGCCGAATATTGGATCTTCAGTTGGAGGACAACAAATG
GGCCTGCAATTGTGACTTATTGCAGTTAAAAACTTGGTTGGAGAACATGCCTCCACAGTCTATAATTG
GTGATGTTGTCTGCAACAGCCCTCCATTTTTTAAAGGAAGTATACTCAGTAGACTAAAGAAGGAATCT
ATTTGCCCTACTCCACCAGTGTATGAAGAACATGAGGATCCTTCAGGATCATTACATCTGGCAGCAAC
ATCTTCAATAAATGATAGTCGCATGTCAACTAAGACCACGTCCATTCTAAAACTACCCACCAAAGCAC
CAGGTTTGATACCTTATATTACAAAGCCATCCACTCAACTTCCAGGACCTTACTGCCCTATTCCTTGT
AACTGCAAAGTCCTATCCCCATCAGGACTTCTAATACATTGTCAGGAGCGCAACATTGAAAGCTTATC
AGATCTGAGACCTCCTCCGCAAAATCCTAGAAAGCTCATTCTAGCGGGAAATATTATTCACAGTTTAA
TGAAGTCTGATCTAGTGGAATATTTCACTTTGGAAATGCTTCACTTGGGAAACAATCGTATTGAAGTT
CAAATTAAGTAAAGGCATGTTCCTTGGTCTCCATAATCTTGAATACTTATATCTTGAATACAATGCCA
TTAAGGAAATACTGCCAGGAACCTTTAATCCAATGCCTAAACTTAAAGTCCTGTATTTAAATAACAAC
CTCCTCCAAGTTTTACCACCACATATTTTTTCAGGGGTTCCTCTAACTAAGGTAAATCTTAAAACAAA
CCAGTTTACCCATCTACCTGTAAGTAATATTTTGGATGATCTTGATTTACTAACCCAGATTGACCTTG
AGGATAACCCCTGGGACTGCTCCTGTGACCTGGTTGGACTGCAGCAATGGATACAAAAGTTAAGCAAG
AACACAGTGACAGATGACATCCTCTGCACTTCCCCCGGGCATCTCGACAAAAAGGAATTGAAAGCCCT
AAATAGTGAAATTCTCTGTCCAGGTTTAGTAAATAACCCATCCATGCCAACACAGACTAGTTACCTTA
TGGTCACCACTCCTGCAACAACAACAAATACGGCTGATACTATTTTACGATCTGTCGACGGC

| | | | |
|---|---|---|---|
| NOV25q, CG55033-12 | | | |
| Protein Sequence | SEQ ID NO: 598 | 580 aa | MW at 64615.9kD |

QTPVLSSRGSCDSLCNCEEKDGTMLINCEAKGIKMVSEISVPPSRPFQLSLLNNGLTMLHTNDFSGLT
NAISIHLGFNNIADIEIGAFNGLGLLKQLHINhNSLEILKEDTFHGLENLEFLQADNNFITVIEPSAF
SKLNRLKVLILNDNAIESLPPNIFRFVPLTHLDLRGNQLQTLPYVGFLEHIGRILDLQLEDNKWACNC
DLLQLKTWLENMPPQSIIGDVVCNSPPFFKGSILSRLKKESICPTPPVYEEHEDPSGSLHLAATSSIN
DSRMSTKTTSILKLPTKAPGLIPYITKPSTQLPGPYCPIPCNCKVLSPSGLLIHCQERNIESLSDLRP
PPQNPRKLILAGNIIHSLMKSDLVEYFTLEMLHLGNNRIEVLEEGSFMNLTRLQKLYLNGNHLTKLSK
GMFLGLHNLEYLYLEYNAIKEILPGTFNPMPKLKVLYLNNNLLQVLPPHIFSGVPLTKVNLKTNQFTH
LPVSNILDDLDLLTQIDLEDNPWDCSCDLVGLQQWIQKLSKNTVTDDILCTSPGHLDKKELKALNSEI
LCPGLVNNPSMPTQTSYLMVTTPATTTNTADTILRS

| | | |
|---|---|---|
| NOV25r, CG55033-13 | SEQ ID NO: 599 | 2481 bp |
| DNA Sequence | ORF Start: at 10 | ORF Stop: at 2473 |

CGCGGATCCCAAACTCCAGTGCTCTCATCCAGAGGCTCTTGTGATTCTCTTTGCAATTGTGAGGAAAA
AGATGGCACAATGCTAATAAATTGTGAAGCAAAAGGTATCAAGATGGTATCTGAAATAAGTGTGCCAC
CATCACGACCTTTCCAACTAAGCTTATTAAATAACGGCTTGACGATGCTTCACACAAATGACTTTTCT
GGGCTTACCAATGCTATTTCAATACACCTTGGATTTAACAATATTGCAGATATTGAGATAGGTGCATT
TAATGGCCTTGGCCTCCTGAAACAACTTCATATCAATCACAATTCTTTAGAAATTCTTAAAGAGGATA
CTTTCCATGGACTGGAAAACCTGGAATTCCTGCAAGCAGATAACAATTTTATCACAGTGATTGAACCA
AGTGCCTTTAGCAAGCTCAACAGACTCAAAGTGTTAATTTTAAATGACAATGCTATTGAGAGTCTTCC
TCCAAACATCTTCCGATTTGTTCCTTTAACCCATCTAGATCTTCGTGGAAATCAATTACAAACATTGC
CTTATGTTGGTTTTCTCGAACACATTGGCCGAATATTGGATCTTCAGTTGGAGGACAACAAATGGGCC
TGCAATTGTGACTTATTGCAGTTAAAAACTTGGTTGGAGAACATGCCTCCACAGTCTATAATTGGTGA
TGTTGTCTGCAACAGCCCTCCATTTTTTAAAGGAAGTATACTCAGTAGACTAAAGAAGGAATCTATTT
GCCCTACTCCACCAGTGTATGAAGAACATGAGGATCCTTCAGGATCATTACATCTGGCAGCAACATCT
TCAATAAATGATAGTCGCATGTCAACTAAGACCACGTCCATTCTAAAACTACCCACCAAAGCACCAGG
TTTGATACCTTATAAATTACAGCCATCCACTCAACTTCCAGGACCTTACTGCCCTATTCCTTGTAACT
GCAAAGTCCTATCCCCATCAGGACTTCTAATACATTGTCAGGAGCGCAACATTGAAAGCTTATCAGAT
GCAAAGTCCTATCCCCATCAGGACTTCTAATACATTGTCAGGAGCGCAACATTGAAAGCTTATCAGAT
CTGAGACCTCCTCCGCAAAATCCTAGAAAGCTCATTCTAGCGGGAAATATTATTCACAGTTTAATGAA
GTCTGATCTAGTGGAATATTTCACTTTGGAAATGCTTCACTTGGGAAACAATCGTATTGAAGTTCTTG
AAGAAGGATCGTTTATGAACCTAACGAGATTACAAAAACTCTATCTAAATGGTAACCACCTGACCAAA
TTAAGTAAAGGCATGTTCCTTGGTCTCCATAATCTTGAATACTTATATCTTGAATACAATGCCATTAA
GGAAATACTGCCAGGAACCTTTAATCCAATGCCTAAACTTAAAGTCCTGTATTTAAATAACAACCTCC
TCCAAGTTTTACCACCACATATTTTTTCAGGGGTTCCTCTAACTAAGGTAAATCTTAAAACAAACCAG
TTTACCCATCTACCTGTAAGTAATATTTTGGATGATCTTGATTTACTAACCCAGATTGACCTTGAGGA
TAACCCCTGGGACTGCTCCTGTGACCTGGTTGGACTGCAGCAATGGATACAAAAGTTAAGCAAGAACA
CAGTGACAGATGACATCCTCTGCACTTCCCCCGGGCATCTCGACAAAAAGGAATTGAAAGCCCTAAAT
AGTGAAATTCTCTGTCCAGGTTTAGTAAATAACCCATCCATGCCAACACAGACTAGTTACCTTATGGT
CACCACTCCTGCAACAACAACAAATACGGCTGATACTATTTTACGATCTCTTACGGACGCTGTGCCAC
TGTCTGTTCTAATATTGGGACTTCTGATTATGTTCATCACTATTGTTTTCTGTGCTGCAGGGATAGTG
GTTCTTGTTCTTCACCGCAGGAGAAGATACAAAAAGAAACAAGTAGATGAGCAAATGAGAGACAACAG
TCCTGTGCATCTTCAGTACAGCATGTATGGCCATAAAACCACTCATCACACTACTGAAAGACCCTCTG
CCTCACTCTATGAACAGCACATGGTGAGCCCCATGGTTCATGTCTATAGAAGTCCATCCTTTGGTCCA
TCTTTTGGAACAGGAAAATCATTCACCACTCACAGGGTCAAATATGAAATACAAAACCACGAACCAAT
CAACAGAATTTTTATCCTTCCAAGATGCCAGCTCATTGTACAGAAACATTTTAGAAAAAGAAAGGGAA
ACATTATCCTGGAGCCCACGAAGAGCTGAAGTTAATGGACATTAATGTACTCACGTCACAAGGAAAGG
TATTAGTGGAACAGACAAAAAATGAGTATTTTGAACTTAAAGCTAATTTACATGCTGAACCTGACTAT
TTAGAAGTCCTGGAGCAGCAAACACTCGAGGCG

| | | | |
|---|---|---|---|
| NOV25r, CG55033-13 | | | |
| Protein Sequence | SEQ ID NO: 600 | 821 aa | MW at 92763.6kD |

QTPVLSSRGSCDSLCNCEEKDGTMLINCEAKGIKMVSEISVPPSRPFQLSLLNNGLTMLHTNDFSGLT
NAISIHLGFNNIADIEIGAFNGLGLLKQLHINHNSLEILKEDTFHGLENLEFLQADNNFITVIEPSAF
SKLNRLKVLILNDNAIESLPPNIFRFVPLTHLDLRGNQLQTLPYVGFLEHIGRILDLQLEDNKWACNC
DLLQLKTWLENMPPQSIIGDVVCNSPPFFKGSILSPLKKESICPTPPVYEEHEDPSGSLHLAATSSIN
DSRNSTKTTSILKLPTKAPGLIPYITKPSTQLPGPYCPIPCNCKVLSPSGLLIHCQERNIESLSDLRP
PPQNPRKLILAGNIIHSLMKSDLVEYFTLEMLHLGNNRIEVLEEGSFMNLTRLQKLYLNGNHLTKLSK
GMFLGLHNLEYLYLEYNAIKEILPGTFNPMPKLKVLYLNNNLLQVLPPHIFSGVPLTKVNLKTNQFTH
LPVSNILDDLDLLTQIDLEDNPWDCSCDLVGLQQWIQKLSKNTVTDDILCTSPGHLDKKELKALNSEI
LCPGLVNNPSMPTQTSYLMVTTPATTTNTADTILRSLTDAVPLSVLILGLLIMFITIVFCAAGIVVLV
LHRRRRYKKKQVDEQMRDNSPVHLQYSMYGHKTTHHTTERPSASLYEQHMVSPMVHVYRSPSFGPKHL
EEEEERNEKEGSDAKHLQRSLLEQENHSPLTGSNMKYKTTNQSTEFLSFQDASSLYRNILEKERELQQ
LGITEYLRKNIAQLQPDMEAHYPGAHEELKLMETLMYSRPRKVLVEQTKNEYFELKANLHAEPDYLEV
LEQQT

| | | |
|---|---|---|
| NOV25s, CG55033-14 | SEQ ID NO: 601 | 2548 bp |
| DNA Sequence | ORF Start: ATG at 14 | ORF Stop: TGA at 2537 |

CACCGGTACCACCATGAAGCTGTGGATTCATCTCTTTTATTCATCTCTCCTTGCCTGTATATCTTTAC
ACTCCCAAACTCCAGTGCTCTCATCCAGAGGCTCTTGTGATTCTCTTTGCAATTGTGAGGAAAAAGAT
GGCACAATGCTAATAAATTGTGAAGCAAAAGGTATCAAGATGGTATCTGAAATAAGTGTGCCACCATC
ACGACCTTTCCAACTAAGCTTATTAAATAACGGCTTGACGATGCTTCACACAAATGACTTTTCTGGGC
TTACCAATGCTATTTCAATACACCTTGGATTTAACAATATTGCAGATATTGAGATAGGTGCATTTAAT
GGCCTTGGCCTCCTGAAACAACTTCATATCAATCACAATTCTTTAGAAATTCTTAAAGAGGATACTTT
CCATGGACTGGAAAACCTGGAATTCCTGCAAGCAGATAACAATTTTATCACAGTGATTGAACCAAGTG
CCTTTAGCAAGCTCAACAGACTCAAAGTGTTAATTTTAAATGACAATGCTATTGAGAGTCTTCCTCCA
AACATCTTCCGATTTGTTCCTTTAACCCATCTAGATCTTCGTGGAAATCAATTACAAACATTGCCTTA
TGTTGGTTTTCTCGAACACATTGGCCGAATATTGGATCTTCAGTTGGAGGACAACAAATGGGCCTGCA
ATTGTGACTTATTGCAGTTAAAAACTTGGTTGGAGAACATGCCTCCACAGTCTATAATTGGTGATGTT
GTCTGCAACAGCCCTCCATTTTTTAAAGGAAGTATACTCAGTAGACTAAAGAAGGAATCTATTTGCCC
TACTCCACCAGTGTATGAAGAACATGAGGATCCTTCAGGATCATTACATCTGGCAGCAACATCTTCAA
TAAATGATAGTCGCATGTCAACTAAGACCACGTCCATTCTAAAACTACCCACCAAAGCACCAGGTTTG
ATACCTTATATTACAAAGCCATCCACTCAACTTCCAGGACCTTACTGCCCTATTCCTTGTAACTGCAA
AGTCCTATCCCCATCAGGACTTCTAATACATTGTCAGGAGCGCAACATTGAAAGCTTATCAGATCTGA
GACCTCCTCCGCAAAATCCTAGAAAGCTCATTCTAGCGGGAAATATTATTCACAGTTTAATGAAGTCT
GATCTAGTGGAATATTTCACTTTGGAAATGCTTCACTTGGGAAACAATCGTATTGAAGTTCTTGAAGA
AGGATCGTTTATGAACCTAACGAGATTGCAAAAACTCTATCTAAATGGTAACCACCTGACCAAATTAA
GTAAAGGCATGTTCCTTGGTCTCCATAATCTTGAATACTTATATCTTGAATACAATGCCATTAAGGAA
ATACTGCCAGGAACCTTTAATCCAATGCCTAAACTTAAAGTCCTGTATTTAAATAACAACCTCCTCCA
AGTTTTACCACCACATATTTTTTCAGGGGTTCCTCTAACTAAGGTAAATCTTAAAACAAACCAGTTTA
CCCATCTACCTGTAAGTAATATTTTGGATGATCTTGATTTACTAACCCAGATTGACCTTGAGGATAAC
CCCTGGGACTGCTCCTGTGACCTGGTTGGACTGCAGCAATGGATACAAAAGTTAAGCAAGAACACAGT
GACAGATGACATCCTCTGCACTTCCCCCGGGCATCTCGACAAAAAGGAATTGAAAGCCCTAAATAGTG
AAATTCTCTGTCCAGGTTTAGTAAATAACCCATCCATGCCAACACAGACTAGTTACCTTATGGTCACC
ACTCCTGCAACAACAACAAATACGGCTGATACTATTTTACGATCTCTTACGGACGCTGTGCCACTGTC
TGTTCTAATATTGGGACTTCTGATTATGTTCATCACTATTGTTTTCTGTGCTGCAGGGATAGTGGTTC
TTGTTCTTCACCGCAGGAGAAGATACAAAAAGAAACAAGTAGATGAGCAAATGAGAGACAACAGTCCT
GTGCATCTTCAGTACAGCATGTATGGCCATAAAACCACTCATCACACTACTGAAAGACCCTCTGCCTC
ACTCTATGAACAGCACATGGTGAGCCCCATGGTTCATGTCTATAGAAGTCCATCCTTTGGTCCAAAGC
ATCTGGAAGAGGAAGAAGAGAGGAATGAGAAAGAAGGAAGTGATGCAAAACATCTCCAAAGAAGTCTT
TTGGAACAGGAAAATCATTCACCACTCACAGGGTCAAATATGAAATACAAAACCACGAACCAATCAAC
AGAATTTTTATCCTTCCAAGATGCCAGCTCATTGTACAGAAACATTTTAGAAAAAGAAAGGGAACTTC
AGCAACTGGGAAATCACAGAATACCTAAGGAAAAACATTGCTCAGCTCCAGCCTGATATGGAGAACAT
TATCCTGGAGCCCACGAAGAGCTGAAGTTAATGGAAACATTAATGTACTCACGTCCAAGGAAGGTATT
AGTGGAACAGACAAAAAATGAGTATTTTGAACTTAAAGCTAATTTACATGCTGAACCTGACTATTTAG
AAGTCCTGGAGCAGCAAACATGACTCGAGGGC

| | | | |
|---|---|---|---|
| NOV25s, CG55033-14 | | | |
| Protein Sequence | SEQ ID NO: 602 | 1841 aa | MW at 95108.5kD |

MKLWIHLFYSSLLACISLHSQTPVLSSRGSCDSLCNCEEKDGTMLINCEAKGIKMVSEISVPPSRPFQ
LSLLNNGLTMLHTNDFSGLTNAISIHLGFNNIADIEIGAFNGLGLLKQLHINHNSLEILKEDTFHGLE
NLEFLQADNNFITVIEPSAFSKLNRLKVLILNDNAIESLPPNIFRFVPLTHLDLRGNQLQTLPYVGFL
EHIGRILDLQLEDNKWACNCDLLQLKTWLENMPPQSIIGDVVCNSPPFFKGSILSRLKKESICPTPPV
YEEHEDPSGSLHLAATSSINDSRMSTKTTSILKLPTKAPGLIPYITKPSTQLPGPYCPIPCNCKVLSP
SGLLIHCQERNIESLSDLRPPPQNPRKLILAGNIIHSLMKSDLVEYFTLEMLHLGNNRIEVLEEGSFM
NLTRLQKLYLNGNHLTKLSKGMFLGLHNLEYLYEYNAIKEILPGATFNPMPKLKVLYLNNNLLQVLPP
HIFSGVPLTKVNLKTNQFTHLPVSNILDDLDLLTQIDLEDNPWDCSCDLVGLQQWIQKLSKNTVTDDI
LCTSPGHLDKKELKALNSEILCPGLVNNPSMPTQTSYLMVTTPATTTNTADTILRSLTDAVPLSVLIL
GLLIMFITIVFCAAGIVVLVLHRRRRYKKKQVDEQMRDNSPVHLQYSMYGHKTTHHTTERPSASLYEQ
HMVSPMVHVYRSPSFGPKHLEEEEERNEKEGSDAKHLQRSLLEQENHSPLTGSNMKYKTTNQSTEFLS
FQDASSLYRNILEKERELQQLGITEYLRKNIAQLQPDMEAHYPGAHEELKLMETLMYSRPRKVLVEQT
KNEYFELKANLHAEPDYLEVLEQQT

A ClustalW comparison of the above protein sequences yields the following sequence alignment shown in Table 25B.

TABLE 25B
Comparison of the NOV25 protein sequences.

NOV25a ------------------------------------------------------------
NOV25b MKLWIHLFYSSLLACISLHSQTPVLSSRGSCDSLCNCEEKDGTMLINCEAKGIKIMVSEIS
NOV25c ------------------------------------------------------------
NOV25d ------------------------------------------------------------
NOV25e ------------------------------------------------------------
NOV25f ----------------RGTKWACNCDLLQLKTWLENMPPQSIIGDVVCNSPPFFKGSILS
NOV25g ----------------RGTKWACNCDLLQLKTWLENMPPQSIIGDVVCNSPPFFKGSILS
NOV25h ------------------------------------------------------------
NOV25i ------------------------------------------------------------
NOV25j ------------------------------------------------------------
NOV25k ------------------------------------------------------------
NOV25l ------------------------------------------------------------
NOV25m ------------------------------------------------------------
NOV25n ------------------------------------------------------------
NOV25o ------------------------------------------------------------
NOV25p ------------------------------------------------------------
NOV25q ------------------------------------------------------------
NOV25r ------------------------------------------------------------
NOV25s ------------------------------------------------------------
NOV25a ----------------------------------------MKLWIHLFYSSLLACISLHS
NOV25b VPPSRPFQLSLLNNGLTMLHTNDFSGLTNAISIHLGFNNIADIEIGAFNGLGLLKQLHIN
NOV25c ------------------------------------------------------------
NOV25d ------------------------------------------------------------
NOV25e ------------------------------------------------------------
NOV25f RLKKESICPTPPVYEEHEDPSGSLHLAATSSINDSRMSTKTTSILKLPTKAPGLIPYITK
NOV25g RLKKESICPTPPVYEEHEDPSGSLHLAATSSINDSRMSTKTTSILKLPTKAPGLIPYITK
NOV25h RLKKESICPTPPVYEEHEDPSGSLHLAATSSINDSRMSTKTTSILKLPTKAPGLIPYITK
NOV25i RLKKESICPTPPVYEEHEDPSGSLHLAATSSINDSRMSTKTTSILKLPTKAPGLIPYITK
NOV25j RLKKESICPTPPVYEEHEDPSGSLHLAATSSINDSRMSTKTTSILKLPTKAPGLIPYITK
NOV25k ----------------------------------------MKLWIHLFYSSLLACISLHS
NOV25l ------------------------------------------------------------
NOV25m ------------------------------------------------------------
NOV25n ----------------------------------------MKLWIHLFYSSLLACISLHS
NOV25o --------------------------------------------DSLFYSSLLACISLHS
NOV25p ------------------------------------------------------------
NOV25q ------------------------------------------------------------
NOV25r ------------------------------------------------------------
NOV25s ----------------------------------------MKLWIHLFYSSLLACISLHS
NOV25a QTPVLSSRGSCDSLCNCEEKDGTMLINCEAKGIKMVSEISVPPSRPFQLSLLNNGLTMLH
NOV25b HNSLEILKEDTFHGLENLEFLQADNNFITVIEPSAFSKLNRLKVLILNDNAIESLPPNIF
NOV25c -----------------------MLINCEAKGIKMVSEISVPPSRPFQLSLLNNGLTMLH
NOV25d ------------------------------------------------------------
NOV25e -------------------TSRTMLINCEAKGIKMVSEISVPPSRPFQLSLLNNGLTMLH
NOV25f PSTQLPGPYCPIPCNCKVLSPSGLLIHCQERNIESLSDLRPPPQNPRKLILAGNIIHSLM
NOV25g PSTQLPGPYCPIPCNCKVLSPSGLLIHCQERNIESLSDLRPPPQNPRKLILAGNIIHSLM
NOV25h -------------------RGTTMLINCEAKGIKMVSEISVPPSRPFQLSLLNNGLTMLH
NOV25i -----------------------MLINCEAKGIKMVSEISVPPSRPFQLSLLNNGLTMLH
NOV25j -----------------------MLINCEAKGIKMVSEISVPPSRPFQLSLLNNGLTMLH
NOV25k QTPVLSSRGSCDSLCNCEEKDGTMLINCEAKGIKMVSEISVPPSRPFQLSLLNNGLTMLH
NOV25l -----------------------MLINCEAKGIKMVSEISVPPSRPFQLSLLNNGLTMLH
NOV25m -----------------------MLINCEAKGIKMVSEISVPPSRPFQLSLLNNGLTMLH
NOV25n QTPVLSSRGSCDSLCNCEEKDGTMLINCEAKGIKMVSEISVPPSRPFQLSLLNNGLTMIH
NOV25o QTPVLSSRGSCDSLCNCEEKDGTMLINCEAKGIKMVSEISVLPSRPFQLSLLNNGLTMLH
NOV25p QTPVLSSRGSCDSLCNCEEKDGTMLINCEAKGIKMVSEISVPPSRPFQLSLLNNGLTMLH
NOV25q QTPVLSSRGSCDSLCNCEEKDGTMLINCEAKGIKMVSEISVPPSRPFQLSLLNNGLTMLH
NOV25r QTPVLSSRGSCDSLCNCEEKDGTMLINCEAKGIKMVSEISVPPSRPFQLSLLNNGLTMLH
NOV25s QTPVLSSRGSCDSLCNCEEKDGTMLINCEAKGIKMVSEISVPPSRPFQLSLLNNGLTMLH
NOV25a TNDFSGLTNAISIHLGFNNIADIEIGAFNGLGLLKQLHINHNSLEILKEDTFHGLENLEF
NOV25b RFVPLTHLDLRGNQLQTLPYVGFLEHIGRILDLQLEDNKWACNCDLLQLKTWLENMPPQS
NOV25c TNDFSGLTNAISIHLGFNNIADIEIGAFNGLGLLKQLHINHNSLEILKEDTFHGLENLEF
NOV25d ------------------------------------------------------------
NOV25e TNDFSGLTNAISIHLGFNNIADIEIGAFNGLGLLKQLHINHNSLEILKEDTFHGLENLEF
NOV25f KSDLVEYFTLEMLHLGNNRIEVLEEGSFMNLTRLQKLYLNGNHLTKLSKGMFLGLHNLEY
NOV25g KSDLVEYFTLEMLHLGNNRIEVLEEGSFMNLTRLQKLYLNGNHLTKLSKGMFLGLHNLEY
NOV25h TNDFSGLTNAISIHLGFNNIADIEIGAFNGLGLLKQLHINHNSLEILKEDTFHGLENLEF
NOV25i TNDFSGLTNAISIHLGFNNIADIEIGAFNGLGLLKQLHINHNSLEILKEDTFHGLENLEF
NOV25j TNDFSGLTNAISIHLGFNNIADIEIGAFNGLGLLKQLHINNNSLEILKEDTFHGLENLEF
NOV25k TNDFSGLTNAISIHLGFNNIADIEIGAFNGLGLLKQLHINHNSLEILKEDTFHGLENLEF
NOV25l TNDFSGLTNAISIHLGFNNIADIEIGAFNGLGLLKQLHINHNSLEILKEDTFHGLENLEF
NOV25m TNDFSGLTNAISIHLGFNNIADIEIGAFNGLGLLKQLHINHNSLEILKEDTFHGLENLEF
NOV25n TNDFSGLTNAISIHLGFNNIADIEIGAFNGLGLLKQLHINHNSLEILKEDTFHGLENLEF
NOV25o TNDFSGLTNAISIHLGFNNIADIEIGAFNGLGLLKQLHINHNSLEILKEDTFHGLENLEF
NOV25p ThDFSGLTNAISIHLGFNNIADIEIGAFNGLGLLKQLHINHNSLEILKEDTFHGLENLEF
NOV25q TNDFSGLTNAISIHLGFNNIADIEIGAFNGLGLLKQLHINNNSLEILKEDTFHGLENLEF
NOV25r TNDFSGLTNAISIHLGFNNIADIEIGAFNGLGLLKQLHINHNSLEILKEDTFHGLENLEF
NOV25s TNDFSGLTNAISIHLGFNNIADIEIGAFNGLGLLKQLHINHNSLEILKEDTFHGLENLEF
NOV25a LQADNNFITVIEPSAFSKLNRLKVLILNDNATESLPPNIFRFVPLTHLDLRGNQLQTLPY
NOV25b IIGDVVCNSPPFFKGSILSRLKKESICPTPPVYEEHEDPSGSLHLAATSSINDSRMSTKT
NOV25c LQADNNFITVIEPSAFSKLNRLKVLILNDNAIESLPPNIFRFVPLTHLDLRGNQLQTLPY
NOV25d ------------------------------------------------------------
NOV25e LQADNNFITVIEPSAFSKLNRLKVLILNDNAIESLPPNIFRFVPLTHLDLRGNQLQTLPY
NOV25f LYLEYNAIKEILPGTFNPMPKLKVLYLNNNLLQVLPPHIFSGVPLTKVNLKTNQFTHLPV
NOV25g LYLEYNAIKEILPGTFNPMPKLKVLYLNNNLLQVLPPHIFSGVPLTKVNLKTNQFTHLPV
NOV25h LQADNNFITVIEPSAFSKLNRLKVLILNDNAIESLPPNIFRFVPLTHLDLRGNQLQTLPY
NOV25i LQADNNFITVIEPSAFSKLNRLKVLILNDNAIESLPPNIFRFVPLTHLDLRGNQLQTLPY
NOV25j LQADNNFITVIEPSAFSKLNRLKVLILNDNAIESLPPNIFRFVPLTHLDLRGNQLQTLPY
NOV25k LQADNNFITVIEPSAFSKLNRLKVLILNDNAIESLPPNIFRFVPLTHLDLRGNQLQTLPY
NOV25l LQADNNFITVIEPSAFSKLNRLKVLILNDNAIESLPPNIFRFVPLTHLDLRGNQLQTLPY
NOV25m LQADNNFITVIEPSAFSKLNRLKVLILNDNAIESLPPNIFRFVPLTHLDLRGNQLQTLPY
NOV25n LQADNNFITVIEPSAFSKLNRLKVLILNDNAIESLPPNIFRFVPLTHLDLRGNQLQTLPY
NOV25o LQADNNFITVIEPSAFSKLNRLKVLILNDNAIESLPPNIFRFVPLTHLDLRGNQLQTLPY
NOV25p LQADNNFITVIEPSAFSKLNRLKVLILNDNAIESLPPNIFRFVPLTHLDLRGNQLQTLPY
NOV25q LQADNNFITVIEPSAFSKLNRLKVLILNDNAIESLPPNIFRFVPLTHLDLRGNQLQTLPY
NOV25r LQADNNFITVIEPSAFSKLNRLKVLILNDNAIESLPPNIFRFVPLTHLDLRGNQLQTLPY
NOV25s LQADNNFITVIEPSAFSKLNRLKVLILNDNAIESLPPNIFRFVPLTHLDLRGNQLQTLPY
NOV25a VGFLEHIGRILDLQLEDNKWACNCDLLQLKTWLENMPPQSIIGDVVCNSPPFFKGSILSR
NOV25b TSILKLPTKAPQIDLEDNPWDCSCDLVGLQQWIQKLSKNTVTDDILCTSPGHLDKKELKA
NOV25c VGFLEHIGRILDLQLEDNKWACNCDLLQLKTWLENMPPQSIIGDVVCNSPPFFKGSILSR
NOV25d ---------------RGTPWDCSCDLVGLQQWIQKLSKNTVTDDILCTSPGHLDKKELKA
NOV25e VGFLEHIGRILDLQLEDNKWACNCDLLQLKTWLENMPPQSIIGDVVCNSPPFFKGSILSR
NOV25f SNILDDLDLLTQIDLEDNPWDCSCDLVGLQQWIQKLSKNTVTDDILCTSPGHLDKKELKA
NOV25g SNILDDLDLLTQIDLEDNPWDCSCDLVGLQQWIQKLSKNTVTDDILCTSPGHLDKKELKA
NOV25h VGFLEHIGRILDLQLEDNKWACNCDLLQLKTWLENMPPQSIIGDVVCNSPPFFKGSILSR
NOV25i VGFLEHIGRILDLQLEDNKWACNCDLLQLKTWLENMPPQSIIGDVVCNSPPFFKGSILSR
NOV25j VGFLEHIGRILDLQLEDNKWACNCDLLQLKTWLENMPPQSIIGDVVCNSPPFFKGSILSR
NOV25k VGFLEHIGRILDLQLEDNKWACNCDLLQLKTWLENMPPQSIIGDVVCNSPPFFKGSILSR
NOV25l VGFLEHIGRILDLQLEDNKWACNCDLLQLKTWLENMPPQSIIGDVVCNSPPFFKGSILSR
NOV25m VGFLEHIGRILDLQLEDNKWACNCDLLQLKTWLENMPPQSIIGDVVCNSPPFFKGSILSR
NOV25n VGFLEHIGRILDLQLEDNKWACNCDLLQLKTWLENMPPQSIIGDVVCNSPPFFKGSILSR
NOV25o VGFLEHIGRILDLQLEDNKWACNCDLLQLKTWLENMPPQSIIGDVVCNSPPFFKGSILSR
NOV25p VGFLEHIGRILDLQLEDNKWACNCDLLQLKTWLENMPPQSIIGDVVCNSPPFFKGSILSR
NOV25q VGFLEHIGRILDLQLEDNKWACNCDLLQLKTWLENMPPQSIIGDVVCNSPPFFKGSILSR
NOV25r VGFLEHIGRILDLQLEDNKWACNCDLLQLKTWLENMPPQSIIGDVVCNSPPFFKGSILSR
NOV25s VGFLEHIGRILDLQLEDNKWACNCDLLQLKTWLENMPPQSIIGDVVCNSPPFFKGSILSR
NOV25a LKKESICPTPPVYEEHEDPSGSLHLAATSSINDSRMSTKTTSILKLPTKAPGLIPYITKP
NOV25b LNSEILCPGLVNNPSMPTQTSYLMVTTPATTTNTADTILRSLTDAVPLSVLILGLLIMFI
NOV25c LKKESICPTPPVYEEHEDPSGSLHIAATSSINDSRMSTKTTSILKLPTKAPGLIPYITKP
NOV25d LNSEILCPGLVNNPSMPTQTSYLMVTTPATTTNTADTILRSLTDAVPLSVLILGLLIMFI
NOV25e LKKESICPTPPVYEEHEDPSGSLHLAATSSINDSRMSTKTTSILKLPTKAPGLIPYITKP
NOV25f LNSEILCPGLVNNPSMPTQTSYLMVTTPATTTNTADTILRSLTDAVPLSVLILGLLIMFI
NOV25g LNSEILCPGLVNNPSMPTQTSYLMVTTPATTTNTADTILRSLTDAVPLSVLILGLLIMFI
NOV25h LKKESICPTPPVYEEHEDPSGSLHLAATSSINDSRMSTKTTSILKLPTKAPGLIPYITKP
NOV25i LKKESICPTPPVYEEHEDPSGSLHLAATSSINDSRMSTKTTSILKLPTKAPGLIPYITKP
NOV25j LKKESICPTPPVYEEHEDPSGSLHLAATSSINDSRISTKTTSILKLPTKAPGLIPYITKP
NOV25k LKKESICPTPPVYEEHEDPSGSLHLAATSSINDSRMSTKTTSILKLPTKAPGLIPYITKP
NOV25l LKKESICPTPPVYEEHEDPSGSLHLAATSSINDSRMSTKTTSILKLPTKAPGLIPYITKP
NOV25m LKKESICPTPPVYEEHEDPSGSLHLAATSSINDSRMSTKTTSILKLPTKAPGLIPYITKP
NOV25n LKKESICPTPPVYEEHEDPSGSLHTAATSSINDSRMSTKTTSILKLPTKAPGLIPYITKP
NOV25o LKKESICPTPPVYEEHEDPSGSLHLAATSSINDSRMSTKTTSILKLPTKAPGLIPYITKP
NOV25p LKKESICPTPPVYEEHEDPSGSLHLAATSSINDSRMSTKTTSILKLPTKAPGLIPYITKP
NOV25q LKKESICPTPPVYEEHEDPSGSLHLAATSSINDSRMSTKTTSILKLPTKAPGLIPYITKP
NOV25r LKKESICPTPPVYEEHEDPSGSLHLAATSSINDSRMSTKTTSILKLPTKAPGLIPYITKP
NOV25s LKKESICPTPPVYEEHEDPSGSLHLAATSSINDSRMSTKTTSILKLPTKAPGLIPYITKP
NOV25a STQLPGPYCPIPCNCKVLSPSGLLIHCQERN---------IESLSDLRPPPQNPRKLILA
NOV25b TIVFCAAGIVVLVLHRRRRYKKKQVDEQMRDNSPVHLQYSMYGHKTTHHTTERPSASLYE
NOV25c STQLPGPYCPIPCNCKVLSPSGLLIHCQERN---------IESLSDLRPPPQNPRKLILA
NOV25d TIVFCAAGIVVLVLHRRRRYKKKQVDEQMRDNSPVHLQYSMYGHKTTHHTTERPSASLYE
NOV25e STQLPGPYCPIPCNCKVLSPSGLLIHCQERN---------IESLSDLRPPPQNPRKLILA
NOV25f TIVFCAAGIVVLVLHRRRRYKKKQVDEQMRDNSPVHLQYSMYGHKTTHHTTERPSASLYE
NOV25g TIVFCAAGIVVLVLHRRRRYKKKQVDEQMRDNSPVHLQYSMYGHKTTHHTTERPSASLYE
NOV25h STQLPGPYCPIPCNCKVLSPSGLLIHCQERN---------IESLSDLRPPPQNPRKLILA
NOV25i STQLPGPYCPIPCNCKVLSPSGLLIHCQEPN---------IESLSDLRPPPQNPRKLILA
NOV25j STQLPGPYCPIPCNCKVLSPSGLLIHCQERN---------IESLSDLRPPPQNPRKLILA
NOV25k STQLPGPYCPIPCNCKVLSPSGLLIHCQERN---------IESLSDLRPPPQNPRKLILA
NOV25l STQLPGPYCPIPCNCKVLSPSGLLIHCQERN---------IESLSDLRPPPQNPRKLILA
NOV25m STQLPGPYCPIPCNCKVLSPSGLLIHCQERN---------IESLSDLRPPPQNPRKLILA
NOV25n STQLPGPYCPIPCNCKVLSPSGLLIHCQERN---------IESLSDLRPPPQNPRXLILA
NOV25o STQLPGPYCPIPCNCKVLSPSGLLIHCQERN---------IESLSDLRPPPQNPRKLILA
NOV25p STQLPGPYCPIPCNCKVLSPSGLLIHCQERN---------IESLSDLRPPPQNPRKLILA
NOV25q STQLPGPYCPIPCNCKVLSPSGLLIHCQERN---------IESLSDLRPPPQNPRKLILA
NOV25r STQLPGPYCPIPCNCKVLSPSGLLIHCQERN---------IESLSDLRPPPQNPRKLILA
NOV25s STQLPGPYCPIPCNCKVLSPSGLLIHCQERN---------IESLSDLRPPPQNPRKLILA
NOV25a GNIIHSLMK---------------------------------------------------
NOV25b QHMVSPMVHVYR------------------------------------------------
NOV25c GNIIHSLMKSDLVEYFTLEMLHLGNNRIEVLEEGSFNNLTRLQKLYLNGNHLTKLSKGMF
NOV25d QHMVSPMVHVYR------------------------------------------------
NOV25e GNIIHSLMKS--------------------------------------------------
NOV25f QHMVSPMVHVYR------------------------------------------------
NOV25g QHMVSPMVHVYR------------------------------------------------
NOV25h GNIIHSLMKSDLVEYFTLEMLHLGNNRIEVLEEGSFTNLTRLQKLYLNGNHLTKLSKGMF
NOV25i GNIIHSLNKS--------------------------------------------------
NOV25j GNIIHSLMKS--------------------------------------------------
NOV25k GNIIHSLMKSDLVEYFTLEMLHLGNNRIEVLEEGSFTNLTRLQKLYLNGNHLTKLSKGMF
NOV25l GNIIHSLMKS--------------------------------------------------
NOV25m GNIIHSLMKS--------------------------------------------------
NOV25n GNIIHSLMKS--------------------------------------------------
NOV25o GNIIHSLMKS--------------------------------------------------
NOV25p GNIIHSLMKS--------------------------------------------------
NOV25q GNIIHSLMKS--------------------------------------------------
NOV25r GNIIHSLMKS--------------------------------------------------
NOV25s GNIIHSLMKS--------------------------------------------------
NOV25a ------------------------------------------------------------
NOV25b ------------------------------------------------------------
NOV25c LGLHNLEYLYLEYNAIKEILPGTFNPMPKLKVLYLNNNLLQVLPPHIFSGVPLTKVNLKT
NOV25d ------------------------------------------------------------
NOV25e ------------------------------------------------------------
NOV25f ------------------------------------------------------------
NOV25g ------------------------------------------------------------
NOV25h LGLHNLEYLYLEYNAIKEILPGTFNPMPKLKVLYLNNNLLQVLPPHIFSGVPLTKVNLKT
NOV25i ------------------------------------------------------------
NOV25j ------------------------------------------------------------
NOV25k LGLHNLEYLYLEYNAIKEILPGTFNPMPKLKVLYLNNNLLQVLPPHIFSGVPLTKVNLKT
NOV25l ------------------------------------------------------------
NOV25m ------------------------------------------------------------
NOV25n ------------------------------------------------------------
NOV25o ------------------------------------------------------------
NOV25p ------------------------------------------------------------
NOV25q ------------------------------------------------------------
NOV25r ------------------------------------------------------------
NOV25s ------------------------------------------------------------
NOV25a ------------------------------------------------------------
NOV25b ------------------------------------------------------------
NOV25c NQFTHLPVSNILDDLDLLTQIDLEDNPWDCSCDLVGLQQWIQKLSKNTVTDDILCTSPGH
NOV25d ------------------------------------------------------------
NOV25e ------------------------------------------------------------
NOV25f ------------------------------------------------------------
NOV25g ------------------------------------------------------------
NOV25h NQFTHLPVSNILDDLDLLTQIDLEDNPWDCSCDLVGLQQWIQKLSKNTVTDDILCTSPGH
NOV25i ------------------------------------------------------------
NOV25j ------------------------------------------------------------
NOV25k NQFTHLPVSNILDDLDLLTQIDLEDNPWDCSCDLVGLQQWIQKLSKNTVTDDILCTSPGH
NOV25l ------------------------------------------------------------
NOV25m ------------------------------------------------------------
NOV25n ------------------------------------------------------------
NOV25o ------------------------------------------------------------
NOV25p ------------------------------------------------------------
NOV25q ------------------------------------------------------------
NOV25r ------------------------------------------------------------
NOV25s ------------------------------------------------------------
NOV25a ------------------------------------------------------------
NOV25b ------------------------------------------------------------
NOV25c LDKKELKALNSEILCPGLVNNPSMPTQTSYLMVTTPATTTNTADTILRSLTDAVPLSVLI
NOV25d ------------------------------------------------------------
NOV25e ------------------------------------------------------------
NOV25f ------------------------------------------------------------
NOV25g ------------------------------------------------------------
NOV25h LDKKELKALNSEILCPGLVNNPSMPTQTSYLNVTTPATTTNTADTILRSLTDAVPLSVLI
NOV25i ------------------------------------------------------------
NOV25j ------------------------------------------------------------
NOV25k LDKKELKALNSEILCPGLVNNPSMPTQTSYLMVTTPATTTNTADTILRSLTDAVPLSVLI
NOV25l ------------------------------------------------------------
NOV25m ------------------------------------------------------------
NOV25n ------------------------------------------------------------
NOV25o ------------------------------------------------------------
NOV25p ------------------------------------------------------------
NOV25q ------------------------------------------------------------
NOV25r ------------------------------------------------------------
NOV25s ------------------------------------------------------------
NOV25a ------------------------------------------------------------
NOV25b ------------------------------------------------------------
NOV25c LGLLIMFITIVFCAAGIVVLVLHRRRRYKKKQVDEQMRDNSPVHLQYSMYGHKTTHHTTE
NOV25d ------------------------------------------------------------
NOV25e ------------------------------------------------------------
NOV25f ------------------------------------------------------------
NOV25g ------------------------------------------------------------
NOV25h LGLLIMFITIVFCAAGIVVLVLHRRRRYKKKQVDEQMRDNSPVHLQYSMYGHKTTHHTTE
NOV25i ------------------------------------------------------------
NOV25j ------------------------------------------------------------
NOV25k LGLLIMFITIVFCAAGIVVLVLHRRRRYKKKQVDEQMRDNSPVHLQYSMYGHKTTHHTTE
NOV25l ------------------------------------------------------------
NOV25m ------------------------------------------------------------
NOV25n ------------------------------------------------------------
NOV25o ------------------------------------------------------------
NOV25p ------------------------------------------------------------
NOV25q ------------------------------------------------------------
NOV25r ------------------------------------------------------------
NOV25s ------------------------------------------------------------
NOV25a --------------------SPSFGPKHLEEEEE---RN----EKEGSDAKHLQRSLLEQ
NOV25b --------------------SPSFGPKHLEEEEE---RN----EKEGSDAKHLQRSLLEQ
NOV25c RPSASLYEQHMVSPMVHVYRSPSFGPKHLEEEEE---RN----EKEGSDAKHLQRSLLEQ
NOV25d --------------------SPSFGPKHLEEEEE---RN----EKEGSDAKHLQRSLLEQ
NOV25e -----------DLVEYFTLEMLHLGNNRIEVLEEGSFMNLTRLQKLYLNGNHLTKLSKGM
NOV25f --------------------SPSFGPKHLEEEEE---RN----EKEGSDAKHLQRSLLEQ
NOV25g --------------------SPSFGPKHLEEEEE---RN----EKEGSDAKHLQRSLLEQ
NOV25h RPSASLYEQHMVSPMVHVYRSPSFGPKHLEEEEE---RN----EKEGSDAKHLQRSLLEQ
NOV25i -----------DLVEYFTLEMLHLGNNRIEVLEEGSFMNLTRLQKLYLNGNHLTKLSKGM
NOV25j -----------DLVEYFTLEMLHLGNNRIEVLEEGSFMNLTRLQKLYLNGNHLTKLSKGM
NOV25k RPSASLYEQHMVSPMVHVYRSPSFGPKHLEEEEE---RN----EKEGSDAKHLQRSLLEQ
NOV25l -----------DLVEYFTLEMLHLGNNRIEVLEEGSFMNLTRLQKLYLNGNHLTKLSKGM
NOV25m -----------DLVEYFTLEMLHLGNNRIEVLEEGSFMNLTRLQKLYLNGNHLTKLSKGM
NOV25n -----------DLVEYFTLEMLHLGNNRIEVLEEGSFMNLTRLQKLYLNGNHLTKLSKGM
NOV25o -----------DLVEYFTLEMLHLGNNRIEVLEEGSFMNLTRLQKLYLNGNHLTKLSKGM
NOV25p -----------DLVEYFTLEMLHLGNNRIEVLEEGSFMNLTRLQKLYLNGNHLTKLSKGM
NOV25q -----------DLVEYFTLEMLHLGNNRIEVLEEGSFMNLTRLQKLYLNGNHLTKLSKGM
NOV25r -----------DLVEYFTLEMLHLGNNRIEVLEEGSFMNLTRLQKLYLNGNHLTKLSKGM
NOV25s -----------DLVEYFTLEMLHLGNNRIEVLEEGSFMNLTRLQKLYLNGNHLTKLSKGM
NOV25a EN----------------------HSPLTGSNMKYKTTNQSTEFLSFQDASSLYRNILEK
NOV25b EN----------------------HSPLTGSNMKYKTTNQSTEFLSFQDASSLYRNILEK
NOV25c EN----------------------HSPLTGSNNKYKTTNQSTEFLSFQDASSLYRNILEK
NOV25d EN----------------------HSPLTGSNMKYKTTNQSTEFLSFQDASSLYENILEK
NOV25e FLGLHNLEYLYLEYNAIKEILPGTFNPMPKLKVLYLNNNLLQVLPPHIFSGVPLTKVNLK
NOV25f EN----------------------HSPLTGSNMKYKTTNQSTEFLSFQDASSLYRNILEK
NOV25g EN----------------------HSPLTGSNMKYKTTNQSTEFLSFQDASSLYRNILEK
NOV25h EN----------------------HSPLTGSNMKYKTTNQSTEFLSFQDASSLYRNILEK
NOV25i FLGLHNLEYLYLEYNAIKEILPGTFNPMPKLKVLYLNNNLLQVLPPHIFSGVPLTKVNLK
NOV25j FLGLHNLEYLYLEYNAIKEILPGTFNPMPKLKVLYLNNNLLQVLPPHIFSGVPLTKVNLK
NOV25k EN----------------------HSPLTGSNMKYKTTNQSTEFLSFQDASSLYRNILEK
NOV25l FLGLHNLEYLYLEYNAIKEILPGTFNPMPKLKVLYLNNNLLQVLPPHIFSGVPLTKVNMC
NOV25m FLGLHNLEYLYLEYNAIKEILPGTFNPMPKLKVLYLNNNLLQVLPPHIFSGVPLTKVNLK
NOV25n FLGLHNLEYLYLEYNAIKEILPGTFNPMPKLKVLYLNNNLLQVLPPHIFSGVPLTKVNLK
NOV25o FLGLHNLEYLYLEYNAIKEILPGTFNPMPKLKVLYLNNNLLQVLPPHIFSGVPLTKVNLK
NOV25p FLGLHNLEYLYLEYNAIKEILPGTFNPMPKLKVLYLNNNLLQVLPPHIFSGVPLTKVNLK
NOV25q FLGLHNLEYLYLEYNAIKEILPGTFNPMPKLKVLYLNNNLLQVLPPHIFSGVPLTKVNLK
NOV25r FLGLHNLEYLYLEYNAIKEILPGTFNPMPKLKVLYLNNNLLQVLPPHIFSOVPLTKVNLK
NOV25s FLGLHNLEYLYLEYNAIKEILPGTFNPMPKLKVLYLNNNLLQVLPPHIFSGVPLTKVNLK
NOV25a ERELQQLGITEYLRKNIAQLQPDMEAHYPGAHEELKLMETLMYSRPR-------------
NOV25b ERELQQLGITEYLRKNIAQLQPDMEAHYPGAHEELKLMETLMYSRPR-------------
NOV25c ERELQQLGITEYLRKNIAQLQPDMEAHYPGAHEELKLMETLMYSRPR-------------
NOV25d ERELQQLGITEYLRKNIAQLQPDMEAHYPGAHEELKLMETLMYSRPR-------------
NOV25e TNQFTHLPVSNILDDLDLLTQIDLEDNPWDCSCDLVGLQQWIQKLSKNTVTDDILCTSPG
NOV25f ERELQQLGITEYLRKNIAQLQPDMEAHYPGAHEELKLMETLMYSRPR-------------
NOV25g ERELQQLGITEYLRKNIAQLQPDMEAHYPGAHEELKLMETLMYSRPR-------------
NOV25h ERELQQLGITEYLRKNIAQLQPDMEAHYPGAHEELKLMETLMYSRPR-------------
NOV25i TNQFTHLPVSNILDDLDLLTQIDLEDNPWDCSCDLVGLQQWIQKLSKNTVTDDILCTSPG
NOV25j TNQFTHLPVSNILDDLDLLTQIDLEDNPWDCSCDLVGLQQWIQKLSKNTVTDDILCTSPG
NOV25k ERELQQLGITEYLRKNIAQLQPDMEAHYPGAHEELKLMETLMYSRPR-------------
NOV25l TNQFTHLPVSNILDDLDLLTQIDLEDNPWDCSCDLVGLQQWIQKLSKNTVTDDILCTSPG
NOV25m TNQFTHLPVSNILDDLDLLTQIDLEDNPWDCSCDLVGLQQWIQKLSKNTVTDDILCTSPG
NOV25n TNQFTHLPVSNILDDLDLLTQIDLEDNPWDCSCDLVGLQQWIQKLSKNTVTDDILCTSPG
NOV25o TNQFTHLPVSNILDDLDLLTQIDLEDNPWDCSCDLVGLQQWIQKLSKNTVTDDILCTSPG
NOV25p TNQFTHLPVSNILDDLDLLTQIDLEDNPWDCSCDLVGLQQWIQKLSKNTVTDDILCTSPG
NOV25q TNQFTHLPVSNILDDLDLLTQIDLEDNPWDCSCDLVGLQQWIQKLSKNTVTDDILCTSPG
NOV25r TNQFTHLPVSNILDDLDLLTQIDLEDNPWDCSCDLVGLQQWIQKLSKNTVTDDILCTSPG
NOV25s TNQFTHLPVSNILDDLDLLTQIDLEDNPWDCSCDLVGLQQWIQKLSKNTVTDDILCTSPG
NOV25a -------KVLVEQTKNEYFELKANLHAEPDYLEVLEQQT---------------------
NOV25b -------KVLVEQTKNEYFELKANLHAEPDYLEVLEQQT---------------------
NOV25c -------KVLVEQTKNEYFELKANLHAEPDYLEVLEQQT---------------------
NOV25d -------KVLVEQTKNEYFELKANLHAEPDYLEVLEQQTLEA------------------
NOV25e HLDKKELKALNSEILCPGLVNNPSMPTQTSYLMVTVDG----------------------
NOV25f -------KVLVEQTKNEYFELKANLHAEPDYLEVLEQQTLEA------------------
NOV25g -------KVLVEQTKNEYFELKANLHAEPDYLEVLEQQTLEA------------------
NOV25h -------KVLVEQTKNEYFELKANLHAEPDYLEVLEQQTLEAKGEFQH------------
NOV25i HLDKKELKALNSEILCPGLVNNPSMPTQTSYLMBT-------------------------
NOV25j HLDKKELKALNSETLCPGLVNNPSMPTQTSYLMVTTPATTTNTADTILR-----------
NOV25k -------KVLVEQTKNEYFELKANLHAEPDYLEVLEQQT---------------------
NOV25l HLDKKELKALNSEILCPGLVNNPSMPTQTSYLMVT-------------------------
NOV25m HLDKKELKALNSEILCPGLVNNPSMPTQTSYLMVT-------------------------
NOV25n HLDKKELKALNSEILCPGLVNNPSMPTQTSYLMVTTPATTTNTADTILRSLTDAVPLSVL
NOV25o HLDKKELKALNSEILCPGLVNNPSMPTQTSYLMVTTPATTTNTADTILRSLTDAVPLSVL
NOV25p HLDKKELKALNSEILCPGLVNNPSMPTQTSYLMVTTPATTTNTADTILRS----------
NOV25q HLDKKELKALNSETLCPGLVNNPSMPTQTSYLMVTTPATTTNTADTILRS----------
NOV25r HLDKKELKALNSEILCPGLVNNPSMPTQTSYLMVTTPATTTNTADTILRSLTDAVPLSVL
NOV25s HLDKKELKALNSEILCPGLVNNPSMPTQTSYLMVTTPATTTNTADTILRSLTDAVPLSVL
NOV25a ------------------------------------------------------------
NOV25b ------------------------------------------------------------
NOV25c ------------------------------------------------------------
NOV25d ------------------------------------------------------------
NOV25e ------------------------------------------------------------
NOV25f ------------------------------------------------------------
NOV25g ------------------------------------------------------------
NOV25h ------------------------------------------------------------
NOV25i ------------------------------------------------------------
NOV25j ------------------------------------------------------------
NOV25k ------------------------------------------------------------
NOV25l ------------------------------------------------------------
NOV25m ------------------------------------------------------------
NOV25n ILGLLIMFITIVFCAAGIVVLVLHRRRRYKKKQVDEQMRDNSPVHLQYSMYGHKTTHHTT
NOV25o ILGLLIMFITIVFCAAGIVVLVLHRRRRYKKKQVDEQMRDNSPVHLQYSMYGHKTTHHTT
NOV25p ------------------------------------------------------------
NOV25q ------------------------------------------------------------
NOV25r ILGLLIMFITIVFCAAGIVVLVLHRRRRYKKKQVDEQMRDNSPVHLQYSMYGHKTTHHTT
NOV25s ILGLLIMFITIVFCAAGIVVLVLHRRRRYKKKQVDEQMRDNSPVHLQYSMYGHKTTHHTT
NOV25a ------------------------------------------------------------
NOV25b ------------------------------------------------------------
NOV25c ------------------------------------------------------------
NOV25d ------------------------------------------------------------
NOV25e ------------------------------------------------------------
NOV25f ------------------------------------------------------------
NOV25g ------------------------------------------------------------
NOV25h ------------------------------------------------------------
NOV25i ------------------------------------------------------------
NOV25j ------------------------------------------------------------
NOV25k ------------------------------------------------------------
NOV25l ------------------------------------------------------------
NOV25m ------------------------------------------------------------
NOV25n ERPSASLYEQHMVSPMVHVYRSPSFGPKHLEEEEERNEKEGSDAKHLQRSLLEQENHSPL
NOV25o ERPSASLYEQHMVSPMVHVYRSPSFGPKHLEEEEERNEKEGSDAKHLQRSLLEQENHSPL
NOV25p ------------------------------------------------------------
NOV25q ------------------------------------------------------------
NOV25r ERPSASLYEQHMVSPMVHVYRSPSFGPKHLEEEEERNEKEGSDAKHLQRSLLEQENHSPL
NOV25s ERPSASLYEQHMVSPMVHVYRSPSFGPKHLEEEEERNEKEGSDAKHLQRSLLEQENHSPL
NOV25a ------------------------------------------------------------
NOV25b ------------------------------------------------------------
NOV25c ------------------------------------------------------------
NOV25d ------------------------------------------------------------
NOV25e ------------------------------------------------------------
NOV25f ------------------------------------------------------------
NOV25g ------------------------------------------------------------
NOV25h ------------------------------------------------------------
NOV25i ------------------------------------------------------------
NOV25j ------------------------------------------------------------
NOV25k ------------------------------------------------------------
NOV25l ------------------------------------------------------------
NOV25m ------------------------------------------------------------
NOV25n TGSNMKYKTTNQSTEFLSFQDASSLYRNILEKERELQQLGITEYLRKNIAQLQPDMEAHY
NOV25o TGSNMKYKTTNQSTEFLSFQDASSLYRNILEKERELQQLGITEYLRKNIAQLQPDMEAHY
NOV25p ------------------------------------------------------------
NOV25q ------------------------------------------------------------
NOV25r TGSNNKYKTTNQSTEFLSFQDASSLYRNILEKERELQQLGITEYLRKNIAQLQPDMEAHY
NOV25s TGSNMKYKTTNQSTEFLSFQDASSLYRNILEKERELQQLGITEYLRKNIAQLQPDMEAHY
NOV25a ---------------------------------------------------
NOV25b ---------------------------------------------------
NOV25c ---------------------------------------------------
NOV25d ---------------------------------------------------
NOV25e ---------------------------------------------------
NOV25f ---------------------------------------------------
NOV25g ---------------------------------------------------
NOV25h ---------------------------------------------------
NOV25i ---------------------------------------------------
NOV25j ---------------------------------------------------
NOV25k ---------------------------------------------------
NOV25l ---------------------------------------------------
NOV25m ---------------------------------------------------
NOV25n PGAHEELKLMETLMYSRPRKVLVEQTKNEYFELKANLHAEPDYLEVLEQQT
NOV25o PGAHEELKLMETLMYSRPRXVLVEQTKNEYFELKANLHAEPDYLEVLEQQT
NOV25p ---------------------------------------------------
NOV25q ---------------------------------------------------
NOV25r PGAHEELKLMETLMYSRPRXVLVEQTKNEYFELKANLHAEPDYLEVLEQQT
NOV25s PGAHEELKLMETLMYSRPRXVLVEQTKNEYFELKANLHAEPDYLEVLEQQT
NOV25a (SEQ ID NO: 566)
NOV25b (SEQ ID NO: 568)
NOV25c (SEQ ID NO: 570)
NOV25d (SEQ ID NO: 572)
NOV25e (SEQ ID NO: 574)
NOV25f (SEQ ID NO: 576)
NOV25g (SEQ ID NO: 578)
NOV25h (SEQ ID NO: 580)
NOV25i (SEQ ID NO: 582)
NOV25j (SEQ ID NO: 584)
NOV25k (SEQ ID NO: 586)
NOV25l (SEQ ID NO: 588)
NOV25m (SEQ ID NO: 590)
NOV25n (SEQ ID NO: 592)
NOV25o (SEQ ID NO: 594)
NOV25p (SEQ ID NO: 596)
NOV25q (SEQ ID NO: 598)
NOV25r (SEQ ID NO: 600)
NOV25s (SEQ ID NO: 602)

Further analysis of the NOV25a protein yielded the following properties shown in Table 25C.

TABLE 25C
Protein Sequence Properties NOV25a

| | |
|---|---|
| SignalP analysis: | Cleavage site between residues 21 and 22 |
| PSORT II analysis: | |

| PSG: | a new signal peptide prediction method | |
|---|---|---|
| | N-region: | length 2; pos. chg 1; neg. chg 0 |
| | H-region: | length 25; peak value 8.53 |

| | |
|---|---|
| | PSG score: 4.13 |
| GvH: | von Heijne's method for signal seq. recognition |
| | GvH score (threshold: −2.1): −3.75 |
| | possible cleavage site: between 15 and 16 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 0 | |
| | number of TMS(s) . . . fixed | |
| | PERIPHERAL | Likelihood = 1.48 (at 3) |

| | ALOM score: 1.48 (number of TMSs: 0) | | | |
|---|---|---|---|---|
| MTOP: | Prediction of membrane topology (Hartmann et al.) | | | |
| | Center position for calculation: 6 | | | |
| | Charge difference: −2.0 C(0.5) − N(2.5) | | | |
| | N >= C: N-terminal side will be inside | | | |
| MITDISC: | discrimination of mitochondrial targeting seq | | | |
| | R content: | 1 | Hyd Moment(75): | 5.82 |
| | Hyd Moment(95): | 9.48 | G content: | 1 |
| | D/E content: | 1 | S/T content: | 8 |

| | |
|---|---|
| | Score: −1.79 |
| Gavel: | prediction of cleavage sites for mitochondrial preseq |
| | R-2 motif at 38 SRG\|SC |

NUCDISC: discrimination of nuclear localization signals

| | |
|---|---|
| | pat4: RPRK (4) at 496 |
| | pat7: none |
| | bipartite: none |
| | content of basic residues: 9.2% |
| | NLS Score: −0.22 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 94.1 |
| COIL: | Lupas's algorithm to detect coiled-coil regions |
| 386 K | 0.97 |
| 387 H | 0.98 |
| 388 L | 0.98 |
| 389 E | 0.98 |
| 390 E | 0.98 |
| 391 E | 0.98 |
| 392 E | 0.98 |
| 393 E | 0.98 |
| 394 R | 0.98 |
| 395 N | 0.98 |
| 396 E | 0.98 |
| 397 K | 0.98 |
| 398 E | 0.98 |
| 399 G | 0.98 |
| 400 S | 0.98 |
| 401 D | 0.98 |
| 402 A | 0.98 |
| 403 K | 0.98 |
| 404 H | 0.98 |
| 405 L | 0.98 |
| 406 Q | 0.98 |
| 407 R | 0.98 |
| 408 S | 0.98 |
| 409 L | 0.98 |
| 410 L | 0.98 |
| 411 E | 0.98 |
| 412 Q | 0.98 |
| 413 E | 0.98 |
| 414 N | 0.98 |
| 415 H | 0.98 |
| 416 S | 0.83 |
| | total: 31 residues |

Final Results (k = 9/23):

52.2%: nuclear
26.1%: extracellular, including cell wall
21.7%: mitochondrial

>> prediction for CG55033-04 is nuc (k = 23)

A search of the NOV25a protein against the Geneseq database, a proprietary database that contains sequences published in patents and patent publication, yielded several homologous proteins shown in Table 25D.

TABLE 25D
Geneseq Results for NOV25a

| | | NOV25a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| ABB53270 | Human polypeptide #10 - Homo | 1 . . . 473 | 401/480 (83%) | 0.0 |
| | sapiens, 841 aa. [WO200181363-A1, | 1 . . . 475 | 412/480 (85%) | |
| | 01-NOV-2001] | | | |
| AAE22222 | Human toll like receptor like molecule 5 | 1 . . . 473 | 401/480 (83%) | 0.0 |
| | (TLR-L5) protein - Homo sapiens, 841 | 1 . . . 475 | 412/480 (85%) | |
| | aa. [WO200220569-A2, 14-MAR-2002] | | | |
| ABJ01956 | 158P1D7 SSH protein sequence SEQ | 1 . . . 473 | 401/480 (83%) | 0.0 |
| | ID No 657 - Unidentified, 841 aa. | 1 . . . 475 | 412/480 (85%) | |
| | [WO200216593-A2, 28-FEB-2002] | | | |
| ABJ05539 | Breast cancer-associated protein 4 - | 1 . . . 473 | 401/480 (83%) | 0.0 |
| | Unidentified, 841 aa. [WO200259377- | 1 . . . 475 | 412/480 (85%) | |
| | A2, 01-AUG-2002] | | | |
| ABG78359 | Human protein, homologous to IGFBP- | 1 . . . 473 | 401/480 (83%) | 0.0 |
| | ALS, designated NOV1a - Homo | 1 . . . 475 | 412/480 (85%) | |
| | sapiens, 841 aa. [WO200226826-A2, | | | |
| | 04-APR-2002] | | | |

In a BLAST search of public sequence databases, the NOV25a protein was found to have homology to the proteins shown in the BLASTP data in Table 25E.

TABLE 25E
Public BLASTP Results for NOV25a

| | | NOV25a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| CAD42380 | Sequence 656 from Patent | 1 . . . 473 | 401/480 | (83%) | 0.0 |
| | WO0216593 - Homo sapiens | 1 . . . 475 | 412/480 | (85%) | |
| | (Human), 841 aa. | | | | |
| Q8BLLO | Hypothetical cysteine-rich flanking | 1 . . . 381 | 339/381 | (88%) | 0.0 |
| | region - Mus musculus (Mouse), | 1 . . . 380 | 358/381 | (92%) | |
| | 425 aa. | | | | |
| Q8C110 | Hypothetical cysteine-rich flanking | 1 . . . 381 | 339/381 | (88%) | 0.0 |
| | region - Mus musculus (Mouse), | 1 . . . 380 | 358/381 | (92%) | |
| | 840 aa. | | | | |
| Q9H5Y7 | Hypothetical protein FLJ22774 - | 44 . . . 381 | 338/338 | (100%) | 0.0 |
| | Homo sapiens (Human), 440 aa. | 1 . . . 338 | 338/338 | (100%) | |
| BAC67207 | Neuronal transmembrane protein | 1 . . . 468 | 189/491 | (38%) | 2e−85 |
| | Slitrk4 - Mus musculus (Mouse), | 30 . . . 493 | 265/491 | (53%) | |
| | 866 aa. | | | | |

PFam analysis predicts that the NOV25a protein contains the domains shown in the Table 25F.

TABLE 25F
Domain Analysis of NOV25a

| | | Identities/ | |
|---|---|---|---|
| | | Similarities | |
| Pfam | NOV25a | for the | Expect |
| Domain | Match Region | Matched Region | Value |
| LRR | 137 . . . 160 | 10/25 (40%) | 0.13 |
| | | 19/25 (76%) | |
| LRRCT | 218 . . . 268 | 17/54 (31%) | 2.6e−08 |
| | | 39/54 (72%) | |

### Example 26

The NOV26 clone was analyzed, and the nucleotide and encoded polypeptide sequences are shown in Table 26A.

TABLE 26A
NOV26 Sequence Analysis

| | | |
|---|---|---|
| NOV26a, CG55117-04 | SEQ ID NO: 603 | 3766 bp |
| DNA Sequence | ORF Start: ATG at 10 | ORF Stop: TGA at 2605 |

TTGCTAGCTATGGCCCTCGTACTCGGCTCCCTGTTGCTGCTGGGGCTGTGCGGGAACTCCTTTTCAGG
AGGGCAGCCTTCATCCACAGATGCTCCTAAGGCTTGGAATTATGAATTGCCTGCAACAAATTATGAGA
CCCAAGACTCCCATAAAGCTGGACCCATTGGCATTCTCTTTGAACTAGTGCATATCTTTCTCTATGTG
GTACAGCCGCGTGATTTCCCAGAAGATACTTTGAGAAAATTCTTACAGAAGGCATATGAATCCAAAAT
TGATTATGACAAGCCAGAAACTGTAATCTTAGGTCTAAAGATTGTCTACTATGAAGCAGGGATTATTC
TATGCTGTGTCCTGGGGCTGCTGTTTATTATTCTGATGCCTCTGGTGAAGTATTTCTTTTGTATGTGT
CGTTGCTGTAACAAATGTGGTGGAGAAATGCACCAGCGACAGAAGGAAAATGGGCCCTTCCTGAGGAA
ATGCTTTGCAATCTCCCTGTTGGTGATTTGTATAATAATAAGCATTGGCATCTTCTATGGTTTTGTGG
CAAATCACCAGGTAAGAACCCGGATCAAAAGGAGTCGGAAACTGGCAGATAGCAATTTCAAGGACTTG
CGAACTCTCTTGAATGAAACTCCAGAGCAAATCAAATATATATTGGCCCAGTACAACACTACCAAGGA
CAAGGCGTTCACAGATCTGAACAGTATCAATTCAGTGCTAGGAGGCGGAATTCTTGACCGACTGAGAC
CCAACATCATCCCTGTTCTTGATGAGATTAAGTCCATGGCAACAGCGATCAAGGAGACCAAAGAGGCG
TTGGAGAACATGAACAGCACCTTGAAGAGCTTGCACCAACAAAGTACACAGCTTAGCAGCAGTCTGAC
CAGCGTGAAAACTAGCCTGCGGTCATCTCTCAATGACCCTCTGTGCTTGGTGCATCCATCAAGTGAAA
CCTGCAACAGCATCAGATTGTCTCTAAGCCAGCTGAATAGCAACCCTGAACTGAGGCAGCTTCCACCC
GTGGATGCAGAACTTGACAACGTTAATAACGTTCTTAGGACAGATTTGGATGGCCTGGTCCAACAGGG
CTATCAATCCCTTAATGATATACCTGACAGAGTACAACGCCAAACCACGACTGTCGTAGCAGGTATCA
AAAGGGTCTTGAATTCCATTGGTTCAGATATCGACAATGTAACTCAGCGTCTTCCTATTCAGGATATA
CTCTCAGCATTCTCTGTTTATGTTAATAACACTGAAAGTTACATCCACAGAAATTTACCTACATTGGA
AGAGTATGATTCATACTGGTGGCTGGGTGGCCTGGTCATCTGCTCTCTGCTGACCCTCATCGTGATTT
TTTACTACCTGGGCTTACTGTGTGGCGTGTGCGGCTATGACAGGCATAACACCCCGACCACCCGAGGC
TGTGTCTCCAACACCGGAGGCGTCTTCCTCATGGTTGGAGTTGGATTAAGTTTCCTCTTTTGCTGGAT
ATTGATGATCATTGTGGTTCTTACCTTTGTCTTTGGTGCAAATGTGGAAAAACTGATCTGTGAACCTT
ACACGAGCAAGGAATTATTCCGGGTTTTGGATACACCCTACTTACTAAATGAAGACTGGGAATACTAT
CTCTCTGGGAAGCTATTTAATAAATCAAAAATGAAGCTCACTTTTGAACAAGTTTACAGTGACTGCAA
AAAAAATAGAGGCACTTACGGCACTCTTCACCTGCAGAACAGCTTCAATATCAGTGAACATCTCAACA
TTAATGAGCATACTGGAAGCATAAGCAGTGAATTGGAAAGTCTGAAGGTAAATCTTAATATCTTTCTG
TTGGGTGCAGCAGGAAGAAAAAACCTTCAGGATTTTGCTGCTTGTGGAATAGACAGAATGAATTATGA
CAGCTACTTGGCTCAGACTGGTAAATCCCCCGCAGGAGTGAATCTTTTATCATTTGCATATGATCTAG
AAGCAAAAGCAAACAGTTTGCCCCCAGGAAATTTGAGGAACTCCCTGAAAAGAGATGCACAAACTATT
AAACAATTCACCAGCAACGAGTCCTTCCTATAGAACAATCACTGAGCACTCTATACCAAAAGCGTCAA
GATACTTCAACGCACAGGGAATGGATTGTTGGAGAGAGTAACTAGGATTCTAGCTTCTCTGGATTTTG
CTCAGAACTTCATCACAAACAATACTTCCTCTGTTATTATTGAGGAAACTAAGAAGTATGGGAGAACA
ATAATAGGATATTTTGAACATTATCTGCAGTGGATCGAGTTCTCTATCAGTGAGAAAGTGGCATCGTG
CAAACCTGTGGCCACCGCTCTAGATACTGCTGTTGATGTCTTTCTGTGTAGCTACATTATCGACCCCT
TGAATTTGTTTTGGTTTGGCATAGGAAAAGCTACTGTATTTTTACTTCCGGCTCTAATTTTTGCGGTA
AAACTGGCTAAGTACTATCGTCGAATGGATTCGGAGGACGTGTACGATGATGTTGAAACTATACCCAT
GAAAAATATGGAAAATGGTAATAATGGTTATCATAAAGATCATGTATATGGTATTCACAATCCTGTTA
TGACAAGCCCATCACAACATTGATAGCTGATGTTGAAACTGCTTGAGCATCAGGATACTCAAAGTGGA
AAGGATCACAGATTTTTGGTAGTTTCTGGGTCTACAAGGACTTTCCAAATCCAGGAGCAACGCCAGTG
GCAACGTAGTGACTCAGGCGGGCACCAAGGCAACGGCACCATTGGTCTCTGGGTAGTGCTTTAAGAAT
GAACACAATCACGTTATAGTCCATGGTCCATCACTATTCAAGGATGACTCCCTCCCTTCCTGTCTATT
TTTGTTTTTTACTTTTTTACACTGAGTTTCTATTTAGACACTACAACATATGGGGTGTTTGTTCCCAT
TGGATGCATTTCTATCAAAACTCTATCAAATGTGATGGCTAGATTCTAACATATTGCCATGTGTGGAG
TGTGCTGAACACACACCAGTTTACAGGAAAGATGCATTTTGTGTACAGTAAACGGTGTATATACCTTT
TGTTACCACAGAGTTTTTTAAACAAATGAGTATTATAGGACTTTCTTCTAAATGAGCTAAATAAGTCA
CCATTGACTTCTTGGTGCTGTTGAAAATAATCCATTTTCACTAAAAGTGTGTGAAACCTACAGCATAT
TCTTCACGCAGAGATTTTCATCTATTATACTTTATCAAAGATTGGCCATGTTCCACTTGGAAATGGCA
TGCAAAAGCCATCATAGAGAAACCTGCGTAACTCCATCTGACAAATTCAAAAGAGAGAGAGAGATCTT
GAGAGAGAAATGCTGTTCGTTCAAAAGTGGAGTTGTTTTAACAGATGCCAATTACGGTGTACAGTTTA
ACAGAGTTTTCTGTTGCATTAGGATAAACATTAATTGGAGTGCAGCTAACATGAGTATCATCAGACTA
GTATCAAGTGTTCTAAAATGAAATATGAGAAGATCCTGTCACAATTCTTAGATCTGGTGTCCAGCATG
GATGAAACCTTTGAGTTTGGTCCCTAAATTTGCATGAAAGCACAAGGTAAATATTCATTTGCTTCAGG
AGTTTCATGTTGGATCTGTCATTATCAAAAGTGATCAGCAATGAAGAACTGGTCGGACAAAATTTAAC
GTTGATGTAATGGAATTCCAGATGTAGGCATTCCCCCCAGGTCTTTTCATGTGCAGATTGCAGTTCTG
ATTCATTTGAATAAAAAGGAACTTGG

| | | | |
|---|---|---|---|
| NOV26a, CG55117-04 | SEQ ID NO: 604 | 865 aa | MW at 97200.8kD |
| Protein Sequence | | | |

MALVLGSLLLLGLCGNSFSGGQPSSTDAPKAWNYELPATNYETQDSHKAGPIGILFELVHIFLYVVQP
RDFPEDTLRKFLQKAYESKIDYDKPETVILGLKIVYYEAGIILCCVLGLLFIILMPLVGYFFCMCRCC
NKCGGEMHQRQKENGPFLRKCFAISLLVICIIISIGIFYGFVANHQVRTRIKRSRKLADSNFKDLRTL
LNETPEQIKYILAQYNTTKDKAFTDLNSINSVLGGGILDRLRPNIIPVLDEIKSMATAIKETKEALEN
MNSTLKSLHQQSTQLSSSLTSVKTSLRSSLNDPLCLVHPSSETCNSIRLSLSQLNSNPELRQLPPVDA
ELDNVNNVLRTDLDGLVQQGYQSLNDIPDRVQRQTTTVVAGIKRVLNSIGSDIDNVTQRLPIQDILSA
FSVYVNNTESYIHRNLPTLEEYDSYWWLGGLVICSLLTLIVIFYYLGLLCGVCGYDRHATPTTRGCVS
NTGGVFLMVGVGLSFLFCWILMIIVVLTFVFGANVEKLICEPYTSKELFRVLDTPYLLNEDWEYYLSG
KLFNKSKMKLTFEQVYSDCKKNRGTYGTLHLQNSFNISEHLNINEHTGSISSELESLKVNLNIFLLGA
AGRKNLQDFAACGIDRMNYDSYLAQTGKSPAGVNLLSFAYDLEAKANSLPPGNLRNSLKRDAQTIKTI
HQQRVLPIEQSLSTLYQSVKILQRTGNGLLERVTRILASLDFAQNFITNNTSSVIIEETKKYGRTIIG
YFEHYLQWIEFSISEKVASCKPVATALDTAVDVFLCSYIIDPLNLFWFGIGKATVFLLPALIFAVKWA
KYYRRMDSEDVYDDVETIPMKNMENGNNGYHKDHVYGIHNPVMTSPSQH

| | | |
|---|---|---|
| NOV26b, CG55117-01 | SEQ ID NO: 605 | 3825 bp |
| DNA Sequence | ORF Start: ATG at 69 | ORF Stop: TGA at 2664 |

GNNNNNNANNNNATTCNTNCANTGNACNNNACCAAGTTCTACCTCATGTTTGGAGGATCTTGCTAGCT
ATGGCCCTCGTACTCGGCTCCCTGTTGCTGCTGGGGCTGTGCGGGAACTCCTTTTCAGGAGGGCAGCC
TTCATCCACAGATGCTCCTAAGGCTTGGAATTATGAATTGCCTGCAACAAATTATGAGACCCAAGACT
CCCATAAAGCTGGACCCATTGGCATTCTCTTTGAACTAGTGCATATCTTTCTCTATGTGGTACAGCCG
CGTGATTTCCCAGAAGATACTTTGAGAAAATTCTTACAGAAGGCATATGAATCCAAAATTGATTATGA
CAAGCCAGAAACTGTAATCTTAGGTCTAAAGATTGTCTACTATGAAGCAGGGATTATTCTATGCTGTG
TCCTGGGGCTGCTGTTTATTATTCTGATGCCTCTGGTGGGGTATTTCTTTTGTATGTGTCGTTGCTGT
AACAAATGTGGTGGAGAAATGCACCAGCGACAGAAGGAAAATGGGCCCTTCCTGAGGAAATGCTTTGC
AATCTCCCTGTTGGTGATTTGTATAATAATAAGCATTGGCATCTTCTATGGTTTTGTGGCAAATCACC
AGGTAAGAACCCGGATCAAAAGGAGTCGGAAACTGGCAGATAGCAATTTCAAGGACTTGCGAACTCTC
TTGAATGAAACTCCAGAGCAAATCAAATATATATTGGCCCAGTACAACACTACCAAGGACAAGGCGTT
CACAGATCTGAACAGTATCAATTCAGTGCTAGGAGGCGGAATTCTTGACCGACTGAGACCCAACATCA
TCCCTGTTCTTGATGAGATTAAGTCCATGGCAACAGCGATCAAGGAGACCAAAGAGGCGTTGGAGAAC
ATGAACAGCACCTTGAAGAGCTTGCACCAACAAAGTACACAGCTTAGCAGCAGTCTGACCAGCGTGAA
AACTAGCCTGCGGTCATCTCTCAATGACCCTCTGTGCTTGGTGCATCCATCAAGTGAAACCTGCAACA
GCATCAGATTGTCTCTAAGCCAGCTGAATAGCAACCCTGAACTGAGGCAGCTTCCACCCGTGGATGCA
GAACTTGACAACGTTAATAACGTTCTTAGGACAGATTTGGATGGCCTGGTCCAACAGGGCTATCAATC
CCTTAATGATATACCTGACAGAGTACAACGCCAAACCACGACTGTCGTAGCAGGTATCAAAAGGGTCT
TGAATTCCATTGGTTCAGATATCGACAATGTAACTCAGCGTCTTCCTATTCAGGATATACTCTCAGCA
TTCTCTGTTTATGTTAATAACACTGAAAGTTACATCCACAGAAATTTACCTACATTGGAAGAGTATGA
TTCATACTGGTGGCTGGGTGGCCTGGTCATCTGCTCTCTGCTGACCCTCATCGTGATTTTTTACTACC
TGGGCTTACTGTGTGGCGTGTGCGGCTATGACAGGCATGCCACCCCGACCACCCGAGGCTGTGTCTCC
AACACCGGAGGCGTCTTCCTCATGGTTGGAGTTGGATTAAGTTTCCTCTTTTGCTGGATATTGATGAT
CATTGTGGTTCTTACCTTTGTCTTTGGTGCAAATGTGGAAAAACTGATCTGTGAACCTTACACGAGCA
AGGAATTATTCCGGGTTTTGGATACACCCTACTTACTAAATGAAGACTGGGAATACTATCTCTCTGGG
AAGCTATTTAATAAATCAAAAATGAAGCTCACTTTTGAACAAGTTTACAGTGACTGCAAAAAAAATAG
AGGCACTTACGGCACTCTTCACCTGCAGAACAGCTTCAATATCAGTGAACATCTCAACATTAATGAGC
ATACTGGAAGCATAAGCAGTGAATTGGAAAGTCTGAAGGTAAATCTTAATATCTTTCTGTTGGGTGCA
GCAGGAAGAAAAAACCTTCAGGATTTTGCTGCTTGTGGAATAGACAGAATGAATTATGACAGCTACTT
GGCTCAGACTGGTAAATCCCCCGCAGGAGTGAATCTTTTATCATTTGCATATGATCTAGAAGCAAAAG
CAAACAGTTTGCCCCCAGGAAATTTGAGGAACTCCCTGA~AAGAGATGCACAAACTATTAAAACAATT
CACCAGCAACGAGTCCTTCCTATAGAACAATCACTGAGCACTCTATACCAAAGCGTCAAGATACTTCA
ACGCACAGGGAATGGATTGTTGGAGAGAGTAACTAGGATTCTAGCTTCTCTGGATTTTGCTCAGAACT
TCATCACAAACAATACTTCCTCTGTTATTATTGAGGAAACTAAGAAGTATGGGAGAACAATAATAGGA
TATTTTGAACATTATCTGCAGTGGATCGAGTTCTCTATCAGTGAGAAAGTGGCATCGTGCAAACCTGT
GGCCACCGCTCTAGATACTGCTGTTGATGTCTTTCTGTGTAGCTACATTATCGACCCCTTGAATTTGT
TTTGGTTTGGCATAGGAAAAGCTACTGTATTTTTACTTCCGGCTCTAATTTTTGCGGTAAAACTGGCT
AAGTACTATCGTCGAATGGATTCGGAGGACGTGTACGATGATGTTGAAACTATACCCATGAAAAATAT
GGAAAATGGTAATAATGGTTATCATAAAGATCATGTATATGGTATTCACAATCCTGTTATGACAAGCC
CATCACAACATTGATAGCTGATGTTGAAACTGCTTGAGCATCAGGATACTCAAAGTGGAAAGGATCAC
AGATTTTTGGTAGTTTCTGGGTCTACAAGGACTTTCCAAATCCAGGAGCAACGCCAGTGGCAACGTAG
TGACTCAGGCGGGCACCAAGGCAACGGCACCATTGGTCTCTGGGTAGTGCTTTAAGAATGAACACAAT
CACGTTATAGTCCATGGTCCATCACTATTCAAGGATGACTCCCTCCCTTCCTGTCTATTTTTGTTTTT
TACTTTTTTACACTGAGTTTCTATTTAGACACTACAACATATGGGGTGTTTGTTCCCATTGGATGCAT
TTCTATCAAAACTCTATCAAATGTGATGGCTAGATTCTAACATATTGCCATGTGTGGAGTGTGCTGAA
CACACACCAGTTTACAGGAAAGATGCATTTTGTGTACAGTAAACGGTGTATATACCTTTTGTTACCAC
AGAGTTTTTTAAACAAATGAGTATTATAGGACTTTCTTCTAAATGAGCTAAATAAGTCACCATTGACT
TCTTGGTGCTGTTGAAAATAATCCATTTTCACTAAAAGTGTGTGAAACCTACAGCATATTCTTCACGC
AGAGATTTTCATCTATTATACTTTATCAAAGATTGGCCATGTTCCACTTGGAAATGGCATGCAAAAGC
CATCATAGAGAAACCTGCGTAACTCCATCTGACAAATTCAAAAGAGAGAGAGAGATCTTGAGAGAGAA
ATGCTGTTCGTTCAAAAGTGGAGTTGTTTTAACAGATGCCAATTACGGTGTACAGTTTAACAGAGTTT
TCTGTTGCATTAGGATAAACATTAATTGGAGTGCAGCTAACATGAGTATCATCAGACTAGTATCAAGT
GTTCTAAAATGAAATATGAGAAGATCCTGTCACAATTCTTAGATCTGGTGTCCAGCATGGATGAAACC
TTTGAGTTTGGTCCCTAAATTTGCATGAAAGCACAAGGTAAATATTCATTTGCTTCAGGAGTTTCATG
TTGGATCTGTCATTATCAAAAGTGATCAGCAATGAAGAACTGGTCGGACAAAATTTAACGTTGATGTA
ATGGAATTCCAGATGTAGGCATTCCCCCCAGGTCTTTTCATGTGCAGATTGCAGTTCTGATTCATTTG
AATAAAAAGGAACTTGG

| | | | |
|---|---|---|---|
| NOV26b, CG55117-01 | SEQ ID NO: 606 | 865 aa | MW at 97200.8kD |
| Protein Sequence | | | |

MALVLGSLLLLGLCGNSFSGGQPSSTDAPKAWNYELPATNYETQDSHKAGPIGILFELVHIFLYVVQP
RDFPEDTLRKFLQKAYESKIDYDKPETVILGLKIVYYEAGIILCCVLGLLFIILMPLVGYFFCNCRCC
NKCGGEMHQRQKENGPFLRKCFAISLLVICIIISIGIFYGFVANHQVRTRIKRSRKLADSNFKDLRTL
LNETPEQIKYILAQYNTTKDKAFTDLNSINSVLGGGILDRLRPNIIPVLDEIKSMATAIKETKEALEN
MNSTLKSLHQQSTQLSSSLTSVKTSLRSSLNDPLCLVHPSSETCNSIRLSLSQLNSNPELRQLPPVDA
ELDNVNNVLRTDLDGLVQQGYQSLNDIPDRVQRQTTTVVAGIKRVLNSIGSDIDNVTQRLPIQDILSA
FSVYVNNTESYIHRNLPTLEEYDSYWWLGGLVICSLLTLIVIFYYLGLLCGVCGYDRHATPTTRGCVS
NTGGVFLMVGVGLSFLFCWILMIIVVLTFVFGANVEKLICEPYTSKELFRVLDTPYLLNEDWEYYLSG
KLFNKSKNKLTFEQVYSDCKKNRGTYGTLHLQNSFNISEHLNINEHTGSISSELESLKVNLNIFLLGA
AGRKNLQDFAACGIDRMNYDSYLAQTGKSPAGVNLLSFAYDLEAKANSLPPGNLRNSLKRNAQTIKTI
HQQRVLPIEQSLSTLYQSVKILQRTGNGLLERVTRILASLDFAQNFITNNTSSVIIEETKKYGRTIIG
YFEHYLQWIEFSISEKVASCKPVATALDTAVDVFLCSYIIDPLNLFWFGIGKATVFLLPALIFAVKLA
KYYRRMDSEDVYDDVETIPMMENGNNGYHKDHVYGIHNPVMTSPSQH

| | | |
|---|---|---|
| NOV26c, CG55117-02 | SEQ ID NO: 607 | 756 bp |
| DNA Sequence | ORF Start: at 1 | ORF Stop: end of sequence |

CAGGTAAGAACCCGGATCAAAAGGAGTCGGAAACTGGCAGATAGCAATTTCAAGGACTTGCGAACTCT
CTTGAATGAAACTCCAGAGCAAATCAAATATATATTGGCCCAGTACAACACTACCAAGGACAAGGCGT
TCACAGATCTGAACAGTATCAATTCAGTGCTAGGAGGCGGAATTCTTGACCGACTGAGACCCAACATC
ATCCCTGTTCTTGATGAGATTAAGTCCATGGCAACAGCGATCAAGGAGACCAAAGAGGCGTTGGAGAA
CATGAACAGCACCTTGAAGAGCTTGCACCAACAAAGTACACAGCTTAGCAGCAGTCTGACCAGCGTGA
AAACTAGCCTGCGGTCATCTCTCAATGACCCTCTGTGCTTGGTGCATCCATCAAGTGAAACCTGCAAC
AGCATCAGATTGTCTCTAAGCCAGCTGAATAGCAACCCTGAACTGAGGCAGCTTCCACCCGTGGATGC
AGAACTTGACAACGTTAATAACGTTCTTAGGACAGATTTGGATGGCCTGGTCCAACAGGGCTATCAAT
CCCTTAATGATATACCTGACAGAGTACAACGCCAAACCACGACTGTCGTAGCAGGTATCAAAAGGGTC
TTGAATTCCATTGGTTCAGATATCGACAATGTAACTCAGCGTCTTCCTATTCAGGATATACTCTCAGC
ATTCTCTGTTTATGTTAATAACACTGAAAGTTACATCCACAGAAATTTACCTACATTGGAAGAGTATG
ATTCATAC

| | | | |
|---|---|---|---|
| NOV26c, CG55117-02 | SEQ ID NO: 608 | 252 aa | MW at 28267.5kD |
| Protein Sequence | | | |

QVRTRIKRSRKLADSNFKDLRTLLNETPEQIKYILAQYNTTKDKAFTDLNSINSVLGGGILDRLRPNI
IPVLDEIKSMATAIKETKEALENMNSTLKSLHQQSTQLSSSLTSVKTSLRSSLNDPLCLVHPSSETCN
SIRLSLSQLNSNPELRQLPPVDAELDNVNNVLRTDLDGLVQQGYQSLNDIPDRVQRQTTTVVAGIKRV
LNSIGSDIDNVTQRLPIQDILSAFSVYVNNTESYIHRNLPTLEEYDSY

| | | |
|---|---|---|
| NOV26d, CG55117-03 | SEQ ID NO: 609 | 765 bp |
| DNA Sequence | ORF Start: at 1 | ORF Stop: end of sequence |

TGTGAACCTTACACGAGCAAGGAATTATTCCGGGTTTTGGATACACCCTACTTACTAAATGAAGACTG
GGAATACTATCTCTCTGGGAAGCTATTTAATAAATCAAAAATGAAGCTCACTTTTGAACAAGTTTACA
GTGACTGCAAAAAAAATAGAGGCACTTACGGCACTCTTCACCTGCAGAACAGCTTCAATATCAGTGAA
TATCTTTCTGTTGGGTGCAGCAGGAAGAAAAAACCTTCAGGATTTTGCTGCTTGTGGAATAGACAGAA
TGAATTATGACAGCTACTTGGCTCAGACTGGTAAATCCCCCGCAGGAGTGAATCTTTTATCATTTGCA
TATGATCTAGAAGCAAAAGCAAACAGTTTGCCCCCAGGAAATTTGAGGAACTCCCTGAAAAGAGATGC
ACAAACTATTAAAACAATTCACCAGCAACGAGTCCTTCCTATAGAACAATCACTGAGCACTCTATACC
AAAGCGTCAAGATACTTCAACGCACAGGGAATGGATTGTTGGAGAGAGTAACTAGGATTCTAGCTTCT
CTGGATTTTGCTCAGAACTTCATCACAAACAATACTTCCTCTGTTATTATTGAGGAAACTAAGAAGTA
TGGGAGAACAATAATAGGATATTTTGAACATTATCTGCAGTGGATCGAGTTCTCTATCAGTGAGAAAG
TGGCATCGTGCAAACCT

| | | | |
|---|---|---|---|
| NOV26d, CG55117-03 | SEQ ID NO: 610 | 255 aa | MW at 28999.6kD |
| Protein Sequence | | | |

CEPYTSKELFRVLDTPYLLNEDWEYYLSGKLFNKSKMKLTFEQVYSDCKKNRGTYGTLHLQNSFNISE
HLNINEHTGSISSELESLKVNLNIFLLGAAGRKNLQDFAACGIDRMNYDSYLAQTGKSPAGVNLLSFA
YDLEAKANSLPPGNLRNSLKRDAQTIKTIHQQRVLPIEQSLSTLYQSVKILQRTGNGLLERVTRILAS
LDFAQNFITNNTSSVIIEETKKYGRTIIGYFEHYLQWIEFSISEKVASCKP

| | | |
|---|---|---|
| NOV26e, CG55117-05 | SEQ ID NO: 611 | 2523 bp |
| DNA Sequence | ORF Start: at 7 | ORF Stop: at 2518 |

GGATCCGGAGGGCAGCCTTCATCCACAGATGCTCCTAAGGCTTGGAATTATGAATTGCCTGCAACAAA
TTATGAGACCCAAGACTCCCATAAAGCTGGACCCATTGGCATTCTCTTTGAACTAGTGCATATCTTTC
TCTATGTGGTACAGCCGCGTGATTTCCCAGAAGATACTTTGAGAAAATTCTTACAGAAGGCATATGAA
TCCAAAATTGATTATGACAAGATTGTCTACTATGAAGCAGGGATTATTCTATGCTGTGTCCTGGGGCT
GCTGTTTATTATTCTGATGCCTCTGGTGGGGTATTTCTTTTGTATGTGTCGTTGCTGTAACAAATGTG
GTGGAGAAATGCACCAGCGACAGAAGGAAAATGGGCCCTTCCTGAGGAAATGCTTTGCAATCTCCCTG
TTGGTGATTTGTATAATAATAAGCATTGGCATCTTCTATGGTTTTGTGGCAAATCACCAGGTAAGAAC
CCGGATCAAAAGGAGTCGGAAACTGGCAGATAGCAATTTCAAGGACTTGCGAACTCTCTTGAATGAAA
CTCCAGAGCAAATCAAATATATATTGGCCCAGTACAACACTACCAAGGACAAGGCGTTCACAGATCTG
AACAGTATCAATTCAGTGCTAGGAGGCGGAATTCTTGACCGACTGAGACCCAACATCATCCCTGTTCT
TGATGAGATTAAGTCCATGGCAACAGCGATCAAGGAGACCAAAGAGGCGTTGGAGAACATGAACAGCA
CCTTGAAGAGCTTGCACCAACAAAGTACACAGCTTAGCAGCAGTCTGACCAGCGTGAAAACTAGCCTG
CGGTCATCTCTCAATGACCCTCTGTGCTTGGTGCATCCATCAAGTGAAACCTGCAACAGCATCAGATT
GTCTCTAAGCCAGCTGAATAGCAACCCTGAACTGAGGCAGCTTCCACCCGTGGATGCAGAACTTGACA
ACGTTAATAACGTTCTTAGGACAGATTTGGATGGCCTGGTCCAACAGGGCTATCAATCCCTTAATGAT
ATACCTGACAGAGTACAACGCCAAACCACGACTGTCGTAGCAGGTATCAAAAGGGTCTTGAATTCCAT
TGGTTCAGATATCGACAATGTAACTCAGCGTCTTCCTATTCAGGATATACTCTCAGCATTCTCTGTTT
ATGTTAATAACACTGAAAGTTACATCCACAGAAATTTACCTACATTGGAAGAGTATGATTCATACTGG
TGGCTGGGTGGCCTGGTCATCTGCTCTCTGCTGACCCTCATCGTGATTTTTTACTACCTGGGCTTACT
GTGTGGCGTGTGCGGCTATGACAGGCATGCCACCCCGACCACCCGAGGCTGTGTCTCCAACACCGGAG
GCGTCTTCCTCATGGTTGGAGTTGGATTAAGTTTCCTCTTTTGCTGGATATTGATGATCATTGTGGTT
CTTACCTTTGTCTTTGGTGCAAATGTGGAAAAACTGATCTGTGAACCTTACACGAGCAAGGAATTATT
CCGGGTTTTGGATACACCCTACTTACTAAATGAAGACTGGGAATACTATCTCTCTGGGAAGCTATTTA
ATAAATCAAAAATGAAGCTCACTTTTGAACAAGTTTACAGTGACTGCAAAAAAAATAGAGGCACTTAC
GGCACTCTTCACCTGCAGAACAGCTTCAATATCAGTGAACATCTCAACATTAATGAGCATACTGGAAG
CATAAGCAGTGAATTGGAAAGTCTGAAGGTAAATCTTAATATCTTTCTGTTGGGTGCAGCAGGAAGAA
AAAACCTTCAGGATTTTGCTGCTTGTGGAATAGACAGAATGGATTATGACAGCTACTTGGCTCAGACT
GGTAAATCCCCCGCAGGAGTGAATCTTTTATCATTTGCATATGATCTAGAAGCAAAAGCAAACAGTTT
GCCCCCAGGAAATTTGAGGAACTCCCTGAAAAGAGATGCACAAACTATTAAAACAATTCACCAGCAAC
GAGTCCTTCCTATAGAACAATCACTGAGCACTCTATACCAAAGCGTCAAGATACTTCAACGCACAGGG
AATGGATTGTTGGAGAGAGTAACTAGGACTCTAGCTTCTCTGGATTTTGCTCAGAACTTCATCACAAA
CAATACTTCCTCTGTTATTATTGAGGAAACTAAGAAGTATGGGAGGACAATAATAGGATATTTTGAAC
ATTATCTGCAGTGGATCGAGTTCTCTATCAGTGAGAAAGTGGCATCGTGCAAACCTGTGGCCACCGCT
CTAGATACTGCTGTTGATGTCTTTCTGTGTAGCTACATTATCGACCCCTTGAATTTGTTTTGGTTTGG
CATAGGAAAAGCTACTGTATTTTTACTTCCGGCTCTAATTTTTGCGGTAAAACTGGCTAAGTACTATC
GTCGAATGGATTCGGAGGACGTGTACGATGATGTTGAAACTATACCCATGAAAAATATGGAAAATGGT
AATAATGGTTATCATAAAGATCATGTATATGGTATTCACAATCCTGTTATGACAAGCCCATCACAACA
TCTCGAG

| | | | |
|---|---|---|---|
| NOV26e, CG55117-05 | SEQ ID NO: 612 | 837 aa | MW at 94348.3kD |
| Protein Sequence | | | |

GGQPSSTDAPKAWNYELPATNYETQDSHKAGPIGILFELVHIFLYVVQPRDFPEDTLRKFLQKAYESK
IDYDKIVYYEAGIILCCVLGLLFIILMPLVGYFFCMCRCCNKCGGEMHQRQKENGPFLRKCFAISLLV
ICIIISIGIFYGFVANHQVRTRIKRSRKLADSNFKDLRTLLNETPEQIKYILAQYNTTKDKAFTDLNS
INSVLGGGILDRLRPNIIPVLDEIKSMATAIKETKEALENMNSTLKSLHQQSTQLSSSLTSVKTSLRS
SLNDPLCLVHPSSETCNSIRLSLSQLNSNPELRQLPPVDAELDNVNNVLRTDLDGLVQQGYQSLNDIP
DRVQRQTTTVVAGIKRVLNSIGSDIDNVTQRLPIQDILSAFSVYVNNTESYIHRNLPTLEEYDSYWWL
GGLVICSLLTLIVIFYYLGLLCGVCGYDRHATPTTRGCVSNTGGVFLMVGVGLSFLFCWILMIIVVLT
FVFGANVEKLICEPYTSKELFRVLDTPYLLNEDWEYYLSGKLFNKSKMKLTFEQVYSDCKKNRGTYGT
LHLQNSFNISEHLNINEHTGSISSELESLKVNLNIFLLGAAGRKNLQDFAACGIDRMDYDSYLAQTGK
SPAGVNLLSFAYDLEAKANSLPPGNLRNSLKRDAQTIKTIHQQRVLPIEQSLSTLYQSVKILQRTGNG
LLERVTRTLASLDFAQNFITNNTSSVIIEETKKYGRTIIGYFEHYLQWIEFSISEKVASCKPVATALD
TAVDVFLCSYIIDPLNLFWFGIGKATVFLLPALIFAVKLAKYYRRMDSEDVYDDVETIPMKNMENGNN
GYHKDHVYGIHNPVMTSPSQH

| | | |
|---|---|---|
| NOV26f, SNP13382440 of | SEQ ID NO: 613 | 3766 bp |
| CG55117-04, DNA Sequence | ORF Start: ATG at 10 | ORF Stop: TGA at 2605 |
| | SNP Pos: 3274 | SNP Change: C to A |

TTGCTAGCTATGGCCCTCGTACTCGGCTCCCTGTTGCTGCTGGGGCTGTGCGGGAACTCCTTTTCAGG
AGGGCAGCCTTCATCCACAGATGCTCCTAAGGCTTGGAATTATGAATTGCCTGCAACAAATTATGAGA
CCCAAGACTCCCATAAAGCTGGACCCATTGGCATTCTCTTTGAACTAGTGCATATCTTTCTCTATGTG
GTACAGCCGCGTGATTTCCCAGAAGATACTTTGAGAAAATTCTTACAGAAGGCATATGAATCCAAAAT
TGATTATGACAAGCCAGAAACTGTAATCTTAGGTCTAAAGATTGTCTACTATGAAGCAGGGATTATTC
TATGCTGTGTCCTGGGGCTGCTGTTTATTATTCTGATGCCTCTGGTGGGGTATTTCTTTTGTATGTGT
CGTTGCTGTAACAAATGTGGTGGAGAAATGCACCAGCGACAGAAGGAAAATGGGCCCTTCCTGAGGAA
ATGCTTTGCAATCTCCCTGTTGGTGATTTGTATAATAATAAGCATTGGCATCTTCTATGGTTTTGTGG
CAAATCACCAGGTAAGAACCCGGATCAAAAGGAGTCGGAAACTGGCAGATAGCAATTTCAAGGACTTG
CGAACTCTCTTGAATGAAACTCCAGAGCAAATCAAATATATATTGGCCCAGTACAACACTACCAAGGA
CAAGGCGTTCACAGATCTGAACAGTATCAATTCAGTGCTAGGAGGCGGAATTCTTGACCGACTGAGAC
CCAACATCATCCCTGTTCTTGATGAGATTAAGTCCATGGCAACAGCGATCAAGGAGACCAAAGAGGCG
TTGGAGAACATGAACAGCACCTTGAAGAGCTTGCACCAACAAAGTACACAGCTTAGCAGCAGTCTGAC
CAGCGTGAAAACTAGCCTGCGGTCATCTCTCAATGACCCTCTGTGCTTGGTGCATCCATCAAGTGAAA
CCTGCAACAGCATCAGATTGTCTCTAAGCCAGCTGAATAGCAACCCTGAACTGAGGCAGCTTCCACCC
GTGGATGCAGAACTTGACAACGTTAATAACGTTCTTAGGACAGATTTGGATGGCCTGGTCCAACAGGG
CTATCAATCCCTTAATGATATACCTGACAGAGTACAACGCCAAACCACGACTGTCGTAGCAGGTATCA
AAAGGGTCTTGAATTCCATTGGTTCAGATATCGACAATGTAACTCAGCGTCTTCCTATTCAGGATATA
CTCTCAGCATTCTCTGTTTATGTTAATAACACTGAAAGTTACATCCACAGAAATTTACCTACATTGGA
AGAGTATGATTCATACTGGTGGCTGGGTGGCCTGGTCATCTGCTCTCTGCTGACCCTCATCGTGATTT
TTTACTACCTGGGCTTACTGTGTGGCGTGTGCGGCTATGACAGGCATGCCACCCCGACCACCCGAGGC
TGTGTCTCCAACACCGGAGGCGTCTTCCTCATGGTTGGAGTTGGATTAAGTTTCCTCTTTTGCTGGAT
ATTGATGATCATTGTGGTTCTTACCTTTGTCTTTGGTGCAAATGTGGAAAAACTGATCTGTGAACCTT
ACACGAGCAAGGAATTATTCCGGGTTTTGGATACACCCTACTTACTAAATGAAGACTGGGAATACTAT
CTCTCTGGGAAGCTATTTAATAAATCAAAAATGAAGCTCACTTTTGAACAAGTTTACAGTGACTGCAA
AAAAAATAGAGGCACTTACGGCACTCTTCACCTGCAGAACAGCTTCAATATCAGTGAACATCTCAACA
TTAATGAGCATACTGGAAGCATAAGCAGTGAATTGGAAAGTCTGAAGGTAAATCTTAATATCTTTCTG
TTGGGTGCAGCAGGAAGAAAAAACCTTCAGGATTTTGCTGCTTGTGGAATAGACAGAATGAATTATGA
CAGCTACTTGGCTCAGACTGGTAAATCCCCCGCAGGAGTGAATCTTTTATCATTTGCATATGATCTAG
AAGCAAAAGCAAACAGTTTGCCCCCAGGAAATTTGAGGAACTCCCTGAAAAGAGATGCACAAACTATT
AAAACAATTCACCAGCAACGAGTCCTTCCTATAGAACAATCACTGAGCACTCTATACCAAAGCGTCAA
GATACTTCAACGCACAGGGAATGGATTGTTGGAGAGAGTAACTAGGATTCTAGCTTCTCTGGATTTTG
CTCAGAACTTCATCACAAACAATACTTCCTCTGTTATTATTGAGGAAACTAAGAAGTATGGGAGAACA
ATAATAGGATATTTTGAACATTATCTGCAGTGGATCGAGTTCTCTATCAGTGAGAAAGTGGCATCGTG
CAAACCTGTGGCCACCGCTCTAGATACTGCTGTTGATGTCTTTCTGTGTAGCTACATTATCGACCCCT
TGAATTTGTTTTGGTTTGGCATAGGAAAAGCTACTGTATTTTTACTTCCGGCTCTAATTTTTGCGGTA
AAACTGGCTAAGTACTATCGTCGAATGGATTCGGAGGACGTGTACGATGATGTTGAAACTATACCCAT
GAAAAATATGGAAAATGGTAATAATGGTTATCATAAAGATCATGTATATGGTATTCACAATCCTGTTA
TGACAAGCCCATCACAACATTGATAGCTGATGTTGAAACTGCTTGAGCATCAGCATACTCAAAGTGGA
AAGGATCACAGATTTTTGGTAGTTTCTGGGTCTACAAGGACTTTCCAAATCCAGGAGCAACGCCAGTG
GCAACGTAGTGACTCAGGCGGGCACCAAGGCAACGGCACCATTGGTCTCTGGGTAGTGCTTTAAGAAT
GAACACAATCACGTTATAGTCCATGGTCCATCACTATTCAAGGATGACTCCCTCCCTTCCTGTCTATT
TTTGTTTTTTACTTTTTTACACTGAGTTTCTATTTAGACACTACAACATATGGGGTGTTTGTTCCCAT
TGGATGCATTTCTATCAAAACTCTATCAAATGTGATGGCTAGATTCTAACATATTGCCATGTGTGGAG
TGTGCTGAACACACACCAGTTTACAGGAAAGATGCATTTTGTGTACAGTAAACGGTGTATATACCTTT
TGTTACCACAGAGTTTTTTAAACAAATGAGTATTATAGGACTTTCTTCTAAATGAGCTAAATAAGTCA
CCATTGACTTCTTGGTGCTGTTGAAAATAATCCATTTTCACTAAAAGTGTGTGAAACCTACAGCATAT
TCTTCACGCAGAGATTTTCATCTATTATACTTTATCAAAGATTGGCCATGTTCCACTTGGAAATGGCA
TGCAAAAGCAATCATAGAGAAACCTGCGTAACTCCATCTGACAAATTCAAAAGAGAGAGAGAGATCTT
GAGAGAGAAATGCTGTTCGTTCAAAAGTGGAGTTGTTTTAACAGATGCCAATTACGGTGTACAGTTTA
ACAGAGTTTTCTGTTGCATTAGGATAAACATTAATTGGAGTGCAGCTAACATGAGTATCATCAGACTA
GTATCAAGTGTTCTAAAATGALATATGAGAAGATCCTGTCACAATTCTTAGATCTGGTGTCCAGCATG
GATGAAACCTTTGAGTTTGGTCCCTAAATTTGCATGAAAGCACAAGGTAAATATTCATTTGCTTCAGG
AGTTTCATGTTGGATCTGTCATTATCAAAAGTGATCAGCAATGAAGAACTGGTCGGACAAAATTTAAC
GTTGATGTAATGGAATTCCAGATGTAGGCATTCCCCCCAGGTCTTTTCATGTGCAGATTGCAGTTCTG
ATTCATTTGAATAAAAAGGAACTTGG

| | | | |
|---|---|---|---|
| NOV26f, SNP13382440 of | SEQ ID NO: 614 | 865 aa | MW at 97200.8kD |
| CG55117-04, Protein | | | SNP Change: no change |
| Sequence | | | |

MALVLGSLLLLGLCGNSFSGGQPSSTDAPKAWNYELPATNYETQDSHKAGPIGILFELVHIFLYVVQP
RDFPEDTLRXFLQKAYESKIDYDKPETVILGLKIVYYEAGIILCCVLGLLFIILMPLVGYFFCMCRCC
NKCGGEMHQRQKENGPFLRKCFAISLLVICIIISIGIFYGFVANHQVRTRIKRSRKLADSNFKDLRTL
LNETPEQIKYILAQYNTTKDKAFTDLNSINSVLGGGILDRLRPNIIPVLDEIKSMATAIKETKEALEN
MNSTLKSLHQQSTQLSSSLTSVKTSLRSSLNDPLCLVHPSSETCNSIRLSLSQLNSNPELRQLPPVDA
ELDNVNNVLRTDLDGLVQQGYQSLNDIPDRVQRQTTTVVAGIKRVLNSIGSDIDNVTQRLPIQDILSA
FSVYVNNTESYIHRNLPTLEEYDSYWWLGGLVICSLLTLIVIFYYLGLLCGVCGYDRHATPTTRGCVS
NTGGVFLMVGVGLSFLFCWILMIIVVLTFVFGANVEKLICEPYTSKELFRVLDTPYLLNEDWEYYLSG
KLFNKSKMKLTFEQVYSDCKKNRGTYGTLHLQNSFNISEHLNINEHTGSISSELESLKVNLNIFLLGA
AGRKNLQDFAACGIDRMNYDSYLAQTGKSPAGVNLLSFAYDLEAKANSLPPGNLRNSLKRDAQTIKTI
HQQRVLPIEQSLSTLYQSVKILQRTGNGLLERVTRILASLDFAQNFITNNTSSVIIEETKKYGRTIIG
YFEHYLQWIEFSISEKVASCKPVATALDTAVDVFLCSYIIDPLNLFWFGIGKATVFLLPALIFAVKLA
KYYRRMDSEDVYDDVETIPMKNMENGNNGYHKDHVYGIHNPVMTSPSQH

| | | |
|---|---|---|
| NOV26g, SNP13382439 of | SEQ ID NO: 615 | 3766 bp |
| CG55117-04, DNA Sequence | ORF Start: ATG at 10 | ORF Stop: TGA at 2605 |
| | SNP Pos: 3685 | SNP Change: G to A |

TTGCTAGCTATGGCCCTCGTACTCGGCTCCCTGTTGCTGCTGGGGCTGTGCGGGAACTCCTTTTCAGG
AGGGCAGCCTTCATCCACAGATGCTCCTAAGGCTTGGAATTATGAATTGCCTGCAACAAATTATGAGA
CCCAAGACTCCCATAAAGCTGGACCCATTGGCATTCTCTTTGAACTAGTGCATATCTTTCTCTATGTG
GTACAGCCGCGTGATTTCCCAGAAGATACTTTGAGAAAATTCTTACAGAAGGCATATGAATCCAAAAT
TGATTATGACAAGCCAGAAACTGTAATCTTAGGTCTAAAGATTGTCTACTATGAAGCAGGGATTATTC
TATGCTGTGTCCTGGGGCTGCTGTTTATTATTCTGATGCCTCTGGTGGGGTATTTCTTTTGTATGTGT
CGTTGCTGTAACAAATGTGGTGGAGAAATGCACCAGCGACAGAAGGAAAATGGGCCCTTCCTGAGGAA
ATGCTTTGCAATCTCCCTGTTGGTGATTTGTATAATAATAAGCATTGGCATCTTCTATGGTTTTGTGG
CAAATCACCAGGTAAGAACCCGGATCAAAAGGAGTCGGAAACTGGCAGATAGCAATTTCAAGGACTTG
CGAACTCTCTTGAATGAAACTCCAGAGCPAATCAAATATATATTGGCCCAGTACAACACTACCAAGGA
CAAGGCGTTCACAGATCTGAACAGTATCAATTCAGTGCTAGGAGGCGGAATTCTTGACCGACTGAGAC
CCAACATCATCCCTGTTCTTGATGAGATTAAGTCCATGGCAACAGCGATCAAGGAGACCAAAGAGGCG
TTGGAGAACATGAACAGCACCTTGAAGAGCTTGCACCAACAAAGTACACAGCTTAGCAGCAGTCTGAC
CAGCGTGAAAACTAGCCTGCGGTCATCTCTCAATGACCCTCTGTGCTTGGTGCATCCATCAAGTGAAA
CCTGCAACAGCATCAGATTGTCTCTAAGCCAGCTGAATAGCAACCCTGAACTGAGGCAGCTTCCACCC

Further analysis of the NOV26a protein yielded the following properties shown in Table 26C.

TABLE 26C
Protein Sequence Properties NOV26a

| | |
|---|---|
| SignalP analysis: | Cleavage site between residues 20 and 21 |
| PSORTII analysis: | |

| PSG: | a new signal peptide prediction method | |
|---|---|---|
| | N-region: | length 0; pos. chg 0; neg. chg 0 |
| | H-region: | length 26; peak value 9.51 |
| | PSG score: | 5.11 |

| | |
|---|---|
| GvH: | von Heijne's method for signal seq. recognition |
| | GvH score (threshold: −2.1): 2.78 |
| | possible cleavage site: between 19 and 20 |

>>> Seems to have a cleavable signal peptide (1 to 19)

| ALOM: | Klein et al's method for TM region allocation | | |
|---|---|---|---|
| | Init position for calculation: 20 | | |
| | Tentative number of TMS(s) for the threshold 0.5: 6 | | |
| | INTEGRAL | Likelihood = | Transmembrane 50-66 |
| | | −3.08 | |
| | INTEGRAL | Likelihood = | Transmembrane 109-125 |
| | | −13.69 | |
| | INTEGRAL | Likelihood = | Transmembrane 158-174 |
| | | −12.90 | |
| | INTEGRAL | Likelihood = | Transmembrane 435-451 |
| | | −8.55 | |
| | INTEGRAL | Likelihood = | Transmembrane 491-507 |
| | | −13.06 | |
| | INTEGRAL | Likelihood = | Transmembrane 797-813 |
| | | −3.13 | |

| | |
|---|---|
| PERIPHERAL | Likelihood = 0.90 (at 772) |

| | |
|---|---|
| | ALOM score: −13.69 (number of TMSs: 6) |
| MTOP: | Prediction of membrane topology (Hartmann et al.) |
| | Center position for calculation: 9 |
| | Charge difference: −2.0 C(−1.0)-N(1.0) |
| | N >= C: N-terminal side will be inside |

>>> membrane topology: type 3a

| MITDISC: | discrimination of mitochondrial targeting seq | | | |
|---|---|---|---|---|
| | R content: | 0 | Hyd Moment(75): | 0.43 |
| | Hyd Moment(95): | 2.59 | G content: | 5 |
| | D/E content: | 1 | S/T content: | 6 |

| | |
|---|---|
| | Score: −6.77 |
| Gavel: | Prediction of cleavage sites for mitochondrial preseq |
| | cleavage site motif not found |

NUCDISC: discrimination of nuclear localization signals

| | |
|---|---|
| | pat4: none |
| | pat7: none |
| | bipartite: none |
| | content of basic residues: 9.5% |
| | NLS Score: −0.47 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs:

Leucine zipper pattern (PS00029): *** found ***
LPPVDAELDNVNNVLRTDLDGL at 335

none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 76.7 |
| COIL: | Lupas's algorithm to detect coiled-coil regions |
| 251 V | 0.95 |
| 252 L | 0.99 |
| 253 D | 0.99 |
| 254 E | 0.99 |
| 255 I | 0.99 |
| 256 K | 0.99 |
| 257 S | 0.99 |
| 258 M | 0.99 |
| 259 A | 0.99 |
| 260 T | 0.99 |
| 261 A | 0.99 |
| 262 I | 0.99 |
| 263 K | 0.99 |
| 264 E | 0.99 |
| 265 T | 0.99 |
| 266 K | 0.99 |
| 267 E | 0.99 |
| 268 A | 0.99 |
| 269 L | 0.99 |
| 270 E | 0.99 |
| 271 N | 0.99 |
| 272 M | 0.99 |
| 273 N | 0.99 |
| 274 S | 0.99 |
| 275 T | 0.99 |
| 276 L | 0.99 |
| 277 K | 0.99 |
| 278 S | 0.99 |
| 279 L | 0.99 |
| 280 H | 0.98 |
| 281 Q | 0.98 |
| 282 Q | 0.97 |
| 283 S | 0.90 |
| 284 T | 0.90 |
| 285 Q | 0.90 |
| 286 L | 0.90 |
| 287 S | 0.90 |
| 288 S | 0.90 |
| 289 S | 0.89 |
| 290 L | 0.89 |
| 291 T | 0.67 |
| 292 S | 0.51 |
| 293 V | 0.51 |
| 294 K | 0.51 |
| | total: 44 residues |

Final Results (k = 9/23):

77.8%: endoplasmic reticulum
22.2%: mitochondrial

>> prediction for CG55117-04 is end (k = 9)

A search of the NOV26a protein against the Geneseq database, a proprietary database that contains sequences published in patents and patent publication, yielded several homologous proteins shown in Table 26D.

TABLE 26D
Geneseq Results for NOV26a

| | | NOV26a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABJ37079 | Human breast cancer/ovarian cancer | 1 . . . 865 | 865/865 (100%) | 0.0 |
| | related protein #55 - Homo sapiens, | 1 . . . 865 | 865/865 (100%) | |
| | 865 aa. [WO2003000012-A2, 03-JAN- | | | |
| | 2003] | | | |
| ABB82955 | Human PROML related protein | 1 . . . 865 | 865/865 (100%) | 0.0 |
| | (GenBank Identifier No. GI#131214442) - | 1 . . . 865 | 865/865 (100%) | |
| | Homo sapiens, 865 aa. | | | |
| | [WO200299047-A2, 12-DEC-2002] | | | |
| ABB82954 | Human PROML related protein | 1 . . . 865 | 865/865 (100%) | 0.0 |
| | (GenBank Identifier No. GI#5174387) - | 1 . . . 865 | 865/865 (100%) | |
| | Homo sapiens, 865 aa. | | | |
| | [WO200299047-A2, 12-DEC-2002] | | | |
| ABB82953 | Human PROML related protein | 1 . . . 865 | 865/865 (100%) | 0.0 |
| | (GenBank Identifier No. GI#7512271) - | 1 . . . 865 | 865/865 (100%) | |
| | Homo sapiens, 865 aa. | | | |
| | [WO200299047-A2, 12-DEC-2002] | | | |
| AAU79943 | Human AC133 antigen-like protein | 1 . . . 865 | 865/865 (100%) | 0.0 |
| | NOV5 - Homo sapiens, 865 aa. | 1 . . . 865 | 865/865 (100%) | |
| | [WO200229038-A2, 11-APR-2002] | | | |

In a BLAST search of public sequence databases, the NOV26a protein was found to have homology to the proteins shown in the BLASTP data in Table 26E.

TABLE 26E
Public BLASTP Results for NOV26a

| | | NOV26a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| O43490 | Prominin-like protein 1 precursor | 1 . . . 865 | 865/865 | (100%) | 0.0 |
| | (Antigen AC133) (CD133 antigen) - | 1 . . . 865 | 865/865 | (100%) | |
| | Homo sapiens (Human), 865 aa. | | | | |
| Q96EN6 | Similar to prominin (Mouse)-like 1 | 1 . . . 865 | 856/865 | (98%) | 0.0 |
| | (AC133-2 antigen) (MSTP061) - | 1 . . . 856 | 856/865 | (98%) | |
| | Homo sapiens (Human), 856 aa. | | | | |
| Q91XN5 | Prominin - Rattus norvegicus (Rat), | 1 . . . 865 | 528/866 | (60%) | 0.0 |
| | 857 aa. | 1 . . . 857 | 681/866 | (77%) | |
| O54990 | Prominin-like protein 1 precursor | 1 . . . 865 | 526/867 | (60%) | 0.0 |
| | (Antigen AC133 homolog) - Mus | 1 . . . 867 | 683/867 | (78%) | |
| | musculus (Mouse), 867 aa. | | | | |
| T08881 | prominin - mouse, 858 aa. | 1 . . . 865 | 521/867 | (60%) | 0.0 |
| | | 1 . . . 858 | 676/867 | (77%) | |

PFam analysis predicts that the NOV26a protein contains the domains shown in the Table 26F.

TABLE 26F
Domain Analysis of NOV26a

| | | | |
|---|---|---|---|
| Pfam | NOV26a | Identities/ | Expect |
| Domain | Match Region | Similarities for | Value |
| | | the Matched Region | |

### Example 27

The MOV27 clone was analyzed, and the nucleotide and encoded polypeptide sequences are shown in Table 27A.

TABLE 27A
NOV27 Sequence Analysis

| | | |
|---|---|---|
| NOV27a, CG55193-04 | SEQ ID NO: 617 | 1336 bp |
| DNA Sequence | ORF Start: ATG at 1 | ORF Stop: TGA at 1327 |

ATGGGTCTGGCCATGGAGCACGGAGGGTCCTACGCTCGGGCGGGGGGCAGCTCTCGGGGCTGCTGGTA
TTACCTGCGCTACTTCTTCCTCTTCGTCTCCCTCATCCAATTCCTCATCATCCTGGGGCTCGTGCTCT
TCATGGTCTATGGCAACGTGCACGTGAGCACAGAGTCCAACCTGCAGGCCACCGAGCGCCGAGCCGAG
GGCCTATACAGTCAGCTCCTAGGGCTCACGGCCTCCCAGTCCAACTTGACCAAGGAGCTCAACTTCAC
CACCCGCGCCAAGGATGCCATCATGCAGATGTGGCTGAATGCTCGCCGCGACCTGGACCGCATCAATG
CCAGCTTCCGCCAGTGCCAGGGTGACCGGGTAATCTACACGAACAATCAGAGGTACATGGCTGCCATC
ATCTTGAGTGAGAAGCAATGCAGAGATCAATTCAAGGACATGAACAAGAGCTGCGATGCCTTGCTCTT
CATGCTGAATCAGAAGGTGAAGACGCTGGAGGTGGAGATAGCCAAGGAGAAGACCATTTGCACTAAGG
ATAAGGAAAGCGTGCTGCTGAACAAACGCGTGGCGGAGGAACAGCTGGTTGAATGCGTGAAAACCCGG
GAGCTGCAGCACCAAGAGCGCCAGCTGGCCAAGGAGCAACTGCAAAAGGTGCAAGCCCTCTGCCTGCC
CCTGGACAAGGACAAGTTTGAGATGGACCTTCGTAACCTGTGGAGGGACTCCATTATCCCACGCAGCC
TGGACAACCTGGGTTACAACCTCTACCATCCCCTGGGCTCGGAATTGGCCTCCATCCGCAGAGCCTGC
GACCACATGCCCAGCCTCATGAGCTCCAAGGTGGAAGGTCAGTGCCGGAGCCTCCGGGCGGATATCGA
ACGCGTGGCCCGCGAGAACTCAGACCTCCAACGCCAGAAGCTGGAAGCCCAGCAGGGCCTGCGGGCCA
GTCAGGAGGCGAAACAGAAGGTGGAGAAGGAGGCTCAGGCCCGGGAGGCCAAGCTCCAAGCTGAATGC
TCCCGGCAGACCCAGCTAGCGCTGGAGGAGAAGGCGGTGCTGCGGAAGGAACGAGACAACCTGGCCAA
GGAGCTGGAAGAGAAGAAGAGGGAGGCGGAGCAGCTCAGGATGGAGCTGGCCATCAGAAACTCAGCCC
TGGACACCTGCATCAAGACCAAGTCGCAGCCGATGATGCCAGTGTCAAGGCCCATGGGCCCTGTCCCC
AACCCCCAGCCCATCGACCCAGCTAGCCTGGAGGAGTTCAAGAGGAAGATCCTGGAGTCCCAGAGGCC
CCCTGCAGGCATCCCTGTAGCCCCATCCAGTGGCTGAGGAGGCT

| | | | |
|---|---|---|---|
| NOV27a, CG55193-04 | SEQ ID NO: 618 | 442 aa | MW at 50568.7kD |
| Protein Sequence | | | |

MGLAMEHGGSYARAGGSSRGCWYYLRYFFLFVSLIQFLIILGLVLFMVYGNVHVSTESNLQATERRAE
GLYSQLLGLTASQSNLTKELNFTTPAKDAIMQMWLNARPDLDRINASFRQCQGDRVIYTNNQRYMAAI
ILSEKQCRDQFKDMNKSCDALLFMLNQKVKTLEVEIAKEKTICTKDKESVLLNKRVAEEQLVECVKTR
ELQHQERQLAKEQLQKVQALCLPLDKDKFEMDLRNLWRDSIIPRSLDNLGYNLYHPLGSELASIRRAC
DHMPSLMSSKVEGQCRSLRADIERVARENSDLQRQKLEAQQGLRASQEAKQKVEKEAQAREAKLQAEC
SRQTQLALEEKAVLRKERDNLAKELEEKKREAEQLRMELAIRNSALDTCIKTKSQPMMPVSRPMGPVP
NPQPIDPASLEEFKRKILESQRPPAGIPVAPSSG

| | | |
|---|---|---|
| NOV27b, 214575880 | SEQ ID NO: 619 | 1188 bp |
| DNA Sequence | ORF Start: at 1 | ORF Stop: end of sequence |

GGATCCAACGTGCACGTGAGCACAGAGTCCAACCTGCAGGCCACCGAGCACCGAGCCGAGGGCCTATA
CAGTCAGCTCCTAGGGCTCACGGCCTCCCAGTCCAACTTGACCAAGGAGCTCAACTTCACCACCCGCG
CCAAGGATGCCATCATGCAGATGTGGCTGAATGCTCGCCGCGACCTGGACCGCATCAATGCCAGCTTC
CGCCAGTGCCAGGGTGACCGGGTCATCTACACGAACAATCAGAGGTACATGGCTGCCATCATCTTGAG
TGAGAAGCAATGCAGAGATCAATTCAAGGACATGAACAAGAGCTGCGATGCCTTGCTCTTCATGCTGA
ATCAGAAGGTGAAGACGCTGGAGGTGGAGATAGCCAAGGAGAAGACCATTTGCACTAAGGATAAGGAA
AGCGTGCTGCTGAACAAACGCGTGGCGGAGGAACAGCTGGTTGAATGCGTGAAAACCCGGGAGCTGCA
GCACCAAGAGCGCCAGCTGGTCAAGGAGCAACTGCAAAAGGTGCAAGCCCTCTGCCTGCCCCTGGACA
AGGACAAGTTTGAGATGGACCTTCGTAACCTGTGGAGGGACTCCATTATCCCACGCAGCCTGGACAAC
CTGGGTTACAACCTCTACCATCCCCTGGGCTCGGAATTGGCCTCCATCCGCAGAGCCTGCGACCACAT
GCCCAGCCTCATGAGCTCCAAGGTGGAGGAGCTGGCCCGGAGCCTCCGGGCGGATATCGAACGCGTGG
TCCGCGAGAACTCAGACCTCCAACGCCAGAAGCTGGAAGCCCAGCAGGGCCTGCGGGCCAGTCAGGAG
GCGAAACAGAAGGTGGAGAAGGAGGCTCAGGCCCGGGAGGCCAAGCTCCAAGCTGAATGCTCCCGGCA
GACCCAGCTAGCGCTGGAGGAGAAGGCGGTGCTGCGGAGGGAACGAGACAACCTGGCCAAGGAGCTGG
AAGAGAAGAAGAGGGAGGCGGAGCAGCTCAGGATGGAGCTGGCCATCAGAAACTCAGCCCTGGACACC
TGCATCAAGACCAAGTCGCAGCCGATGATGCCAGTGTCAAGGCCCATGGGCCCTGTCCCCAACCCCCA
GCCCATCGACCCAGCTAGCCTGGAGGAGTTCAAGAGGAAGATCCTGGAGTCCCAGAGGCCCCCTGCAG
GCATCCCTGTAGCCCCATCCAGTGGCCTCGAG

| | | | |
|---|---|---|---|
| NOV27b, 214575880 | SEQ ID NO: 620 | 396 aa | MW at 45387.5kD |
| Protein Sequence | | | |

GSNVHVSTESNLQATEHRAEGLYSQLLGLTASQSNLTKELNFTTRAKDAIMQMWLNARRDLDRINASF
RQCQGDRVIYTNNQRYMAAIILSEKQCRDQFKDMNKSCDALLFMLNQKVKTLEVEIAKEKTICTKDKE
SVLLNKRVAEEQLVECVKTRELQHQERQLVKEQLQKVQALCLPLDKDKFEMDLRNLWRDSIIPRSLDN
LGYNLYHPLGSELASIRRACDHMPSLMSSKVEELARSLRADIERVVRENSDLQRQKLEAQQGLRASQE
AKQKVEKEAQAREAKLQAECSRQTQLALEEKAVLRRERDNLAKELEEKKREAEQLRMELAIRNSALDT
CIKTKSQPMMPVSRPMGPVPNPQPIDPASLEEFKRKILESQRPPAGIPVAPSSGLE

| | | |
|---|---|---|
| NOV27c, 214458684 | SEQ ID NO: 621 | 1419 bp |
| DNA Sequence | ORF Start: at 1 | ORF Stop: end of sequence |

GAGATTCCCCCCCCTCGGGTCTCTTGGGCCTGTCCATGCGCCCCCCCCTCCGTGCTCCATGGCCAGAC
CCATGGATCCATGGGTCTGGCCATGGAGCACGGAGGGTCCTACGCTCGGGCGGGGGGCAGCTCTCGGG
GCTGCTGGTATTACCTGCGCTACTTCTTCCTCTTCGTCTCCCTCATCCAATTCCTCATCATCCTGGGG
CTCGTGCTCTTCATGGTCTATGGCAACGTGCACGTGAGCACAGAGTCCAACCTGCAGGCCACCGAGCG
CCGAGCCGAGGGCCTATACAGTCAGCTCCTAGGGCTCACGGCCTCCCAGTCCAACTTGACCAAGGAGC
TCAACTTCACCACCCGCGCCAAGGATGCCATCATGCAGATGTGGCTGAATGCTCGCCGCGACCTGGAC
CGCATCAATGCCAGCTTCCGCCAGTGCCAGGGTGACCGGGTCATCTACACGAACAATCAGAGGTACAT
GGCTGCCATCATCTTGAGTGAGAAGCAATGCAGAGATCAATTCAAGGACATGAACAAGAGCTGCGATG
CCTTGCTCTTCATGCTGAATCAGAAGGTGAAGACGCTGGAGGTGGAGATAGCCAAGGAGAAGACCATT
TGCACTAAGGATAAGGAAAGCGTGCTGCTGAACAAACGCGTGGCGGAGGAACAGCTGGTTGAATGCGT
GAAAACCCGGGAGCTGCAGCACCAAGAGCGCCAGCTGGCCAAGGAGCAACTGCAAAAGGTGCAAGCCC
TCTGCCTGCCCCTGGACAAGGACAAGTTTGAGATGGACCTTCGTAACCTGTGGAGGGACTCCATTATC
CCACGCAGCCTGGACAACCTGGGTTACAACCTCTACCATCCCCTGGGCTCGGAATTGGCCTCCATCCG
CAGAGCCTGCGACCACATGCCCAGCCTCATGAGCTCCAAGGTGGAGGAGCTGGCCCGGAGCCTCCGGG
CGGATATCGAACGCGTGGCCCGCGAGAACTCAGACCTCCAACGCCAGAAGCTGGAAGCCCAGCAGGGC
CTGCGGGCCAGTCAGGAGGCGAAACAGAAGGTGGAGAAGGAGGCTCAGGCCCGGGAGGCCAAGCTCCA
AGCTGAATGCTCCCGGCAGACCCAGCTAGCGCTGGAGGAGAAGGCGGTGCTGCGGAAGGAACGAGACA
ACCTGGCCAAGGAGCTGGAAGAGAAGAAGAGGGAGGCGGAGCAGCTCAGGATGGAGCTGGCCATCAGA
AACTCAGCCCTGGACACCTGCATCAAGACCAAGTCGCAGCCGATGATGCCAGTGTCAAGGCCCATGGG
CCCTGTCCCCAACCCCCAGCCCATCGACCCAGCTAGCCTGGAGGAGTTCAAGAGGAAGATCCTGGAGT
CCCAGAGGCCCCCTGCAGGCATCCCTGTAGCCCCATCCAGTGGCCTCGAG

| | | | |
|---|---|---|---|
| NOV27c, 214458684 | SEQ ID NO: 622 | 470 aa | MW at 53542.0kD |
| Protein Sequence | | | |

EIPPPRVSWACPCAPPSVLHGQTHGSMGLAMEHGGSYARAGGSSRGCWYYLRYFFLFVSLIQFLIILG
LVLFMVYGNVHVSTESNLQATERRAEGLYSQLLGLTASQSNLTKELNFTTRAKDAIMQMWLNARRDLD
RINASFRQCQGDRVIYTNNQRYMAAIILSEKQCRDQFKDMNKSCDALLFMLNQKVKTLEVEIAKEKTI
CTKDKESVLLNKRVAEEQLVECVKTRELQHQERQLAKEQLQKVQALCLPLDKDKFEMDLRNLWRDSII
PRSLDNLGYNLYHPLGSELASIRRACDHMPSLMSSKVEELARSLRADIERVARENSDLQRQKLEAQQG
LRASQEAKQKVEKEAQAREAKLQAECSRQTQLALEEKAVLRKERDNLAKELEEKKREAEQLRMELAIR
NSALDTCIKTKSQPMMPVSRPMGPVPNPQPIDPASLEEFKRKILESQRPPAGIPVAPSSGLE

| | | |
|---|---|---|
| NOV27d, 214458688 | SEQ ID NO: 623 | 1404 bp |
| DNA Sequence | ORF Start: at 1 | ORF Stop: end of sequence |

TTCGCCCTTGGATCCATGGGTCTGGCCATGGAGCACGGAGGGTCCTACGCTCGGGCGGGGGGCAGCTC
TCGGGGCTGCTGGTATTACCTGCGCTACTTCTTCCTCTTCGTCTCCCTCATCCAATTCCTCATCATCC
TGGGGCTCGTGCTCTTCATGGTCTATGGCAACGTGCACGTGAGCACAGAGTCCAACCTGCAGGCCACC
GAGCGCCGAGCCGAGGGCCTATACAGTCAGCTCCTAGGGCTCACGGCCTCCCAGTCCAACTTGACCAA
GGAGCTCAACTTCACCACCCGCGCCAAGGATGCCATCATGCAGATGTGGCTGAATGCTCGCCGCGACC
TGGACCGCATCAATGCCAGCTTCCGCCAGTGCCAGGGTGACCGGGTCATCTACACGAACAATCAGAGG
TACATGGCTGCCATCATCTTGAGTGAGAAGCAATGCAGAGATCAATTCAAGGACATGAACAAGAGCTG
CGATGCCTTGCTCTTCATGCTGAATCAGAAGGTGAAGACGCTGGAGGTGGAGATAGCCAAGGAGAAGA
CCATTTGCACTAAGGATAAGGAAAGCGTGCTGCTGAACAAACGCGTGGCGGAGGAACAGCTGGTTGAA
TGCGTGAAAACCCGGGAGCTGCGGCACCAAGAGCGCCAGCTGGCCAAGGAGCAACTGCAAAAGGTGCA
AGCCCTCTGCCTGCCCCTGGACAAGGACAAGTTTGAGATGGACCTTCGTAACCTGTGGAGGGACTCCA
TTATCCCACGCAGCCTGGACAACCTGGGTTACAACCTCTACCATCCCCTGGGCTCGGAATTGGCCTCC
ATCCGCAGAGCCTGCGACCACATGCCCAGCCTCATGAGCTCCAAGGTGGAGGAGCTGGCCCGGAGCCT
CCGGGCGGATATCGAACGCGTGGCCCGCGAGAACTCAGACCTCCAACGCCAGAAGCTGGAAGCCCAGC
AGGGCCTGCGGGCCAGTCAGGAGGCGAAACAGAAGGTGGAGAAGGAGGCTCAGGCCCGGGAGGCCAAG
CTCCAAGCTGAATGCTCCCGGCAGACCCAGCTAGCGCTGGAGGAGAAGGCGGTGCTGCGGAAGGAACG
AGACAACCTGGCCAAGGAGCTGGAAGAGAAGAAGAGGGAGGCGGAGCAGCTCAGGATGGAGCTGGCCA
TCAGAAACTCAGCCCTGGACACCTGCATCAAGACCAAGTCGCAGCCGATGATGCCAGTGTCAAGGCCC
ATGGGCCCTGTCCCCAACCCCCAGCCCATCGACCCAGCTAGCCTGGAGGAGTTCAAGAGGAAGATCCT
GGAGTCCCAGAGGCCCCCTGCAGGCATCCCTGTAGCCCCATCCAGTGGCCTCGAGGCCACTGGATGGG
GCTACAGGGATGCCTCCGTGCTCCATGGCCAGACCCATGGATCC

| | | | |
|---|---|---|---|
| NOV27d, 214458688 | SEQ ID NO: 624 | 468 aa | MW at 53321.6kD |
| Protein Sequence | | | |

FALGSMGLAMEHGGSYARAGGSSRGCWYYLRYFFLFVSLIQFLITLGLVLFMVYGNVHVSTESNLQAT
ERRAEGLYSQLLGLTASQSNLTKELNFTTRAKDAIMQMWLNARRDLDRINASFRQCQGDRVIYTNNQR
YMAAIILSEKQCRDQFKDMNKSCDALLFMLNQKVKTLEVEIAKEKTICTKDKESVLLNKRVAEEQLVE
CVKTRELRHQERQLAKEQLQKVQALCLPLDKDKFEMDLRNLWRDSIIPRSLDNLGYNLYHPLGSELAS
IRRACDHMPSLMSSKVEELARSLRADIERVARENSDLQRQKLEAQQGLRASQEAKQKVEKEAQAREAK
LQAECSRQTQLALEEKAVLRKERDNLAKELEEKKREAEQLRMELAIRNSALDTCIKTKSQPMMPVSRP
MGPVPNPQPIDPASLEEFKRKILESQRPPAGIPVAPSSGLEATGWGYPDASVLHGQTHGS

| | | |
|---|---|---|
| NOV27e, CG55193-01 | SEQ ID NO: 625 | 1366 bp |
| DNA Sequence | ORF Start: ATG at 1 | ORF Stop: TGA at 1327 |

ATGGGTCTGGCCATGGAGCACGGAGGGTCCTACGCTCGGGCGGGGGGCAGCTCTCGGGGCTGCTGGTA
TTACCTGCGCTACTTCTTCCTCTTCGTCTCCCTCATCCAATTCCTCATCATCCTGGGGCTCGTGCTCT
TCATGGTCTATGGCAACGTGCACGTGAGCACAGAGTCCAACCTGCAGGCCACCGAGCGCCGAGCCGAG
GGCCTATACAGTCAGCTCCTAGGGCTCACGGCCTCCCAGTCCAACTTGACCAAGGAGCTCAACTTCAC
CACCCGCGCCAAGGATGCCATCATGCAGATGTGGCTGAATGCTCGCCGCGACCTGGACCGCATCAATG
CCAGCTTCCGCCAGTGCCAGGGTGACCGGGTAATCTACACGAACAATCAGAGGTACATGGCTGCCATC
ATCTTGAGTGAGAAGCAATGCAGAGATCAATTCAAGGACATGAACAAGAGCTGCGATGCCTTGCTCTT
CATGCTGAATCAGAAGGTGAAGACGCTGGAGGTGGAGATAGCCAAGGAGAAGACCATTTGCACTAAGG
ATAAGGAAAGCGTGCTGCTGAACAAACGCGTGGCGGAGGAACAGCTGGTTGAATGCGTGAAAACCCGG
GAGCTGCAGCACCAAGAGCGCCAGCTGGCCAAGGAGCAACTGCAAAAGGTGCAAGCCCTCTGCCTGCC
CCTGGACAAGGACAAGTTTGAGATGGACCTTCGTAACCTGTGGAGGGACTCCATTATCCCACGCAGCC
TGGACAACCTGGGTTACAACCTCTACCATCCCCTGGGCTCGGAATTGGCCTCCATCCGCAGAGCCTGC
GACCACATGCCCAGCCTCATGAGCTCCAAGGTGGAAGGTCAGTGCCGGAGCCTCCGGGCGGATATCGA
ACGCGTGGCCCGCGAGAACTCAGACCTCCAACGCCAGAAGCTGGAAGCCCAGCAGGGCCTGCGGGCCA
GTCAGGAGGCGAAACAGAAGGTGGAGAAGGAGGCTCAGGCCCGGGAGGCCAAGCTCCAAGCTGAATGC
TCCCGGCAGACCCAGCTAGCGCTGGAGGAGAAGGCGGTGCTGCGGAAGGAACGAGACAACCTGGCCAA
GGAGCTGGAAGAGAAGAAGAGGGAGGCGGAGCAGCTCAGGATGGAGCTGGCCATCAGAAACTCAGCCC
TGGACACCTGCATCAAGACCAAGTCGCAGCCGATGATGCCAGTGTCAAGGCCCATGGGCCCTGTCCCC
AACCCCCAGCCCATCGACCCAGCTAGCCTGGAGGAGTTCAAGAGGAAGATCCTGGAGTCCCAGAGGCC
CCCTGCAGGCATCCCTGTAGCCCCATCCAGTGGCTGAGGAGGCTCCGGCACTGACCTAAGGGCGAATC
CCAGGA

| | | | |
|---|---|---|---|
| NOV27e, CG55193-01 | SEQ ID NO: 626 | 442 aa | MW at 50568.7kD |
| Protein Sequence | | | |

MGLAMEHGGSYARAGGSSRGCWYYLRYFFLFVSLIQFLIILGLVLFMVYGNVHVSTESNLQATERPAE
GLYSQLLGLTASQSNLTKELNFTTRAKDAIMQMWLNARRDLDRINASFRQCQGDRVIYTNNQRYMAAI
ILSEKQCRDQFKDMNKSCDALLFMLNQKVKTLEVEIAKEKTICTKDKESVLLNKRVAEEQLVECVKTR
ELQHQERQLAKEQLQKVQALCLPLDKDKFEMDLRNLWRDSIIPRSLDNLGYNLYHPLGSELASIRRAC
DHMPSLMSSKVEGQCRSLRADIERVARENSDLQRQKLEAQQGLHASQEAKQKVEKEAQAREAKLQAEC
SRQTQLALEEKAVLRKERDNLAKELEEKKREAEQLRMELAIRNSALDTCIKTKSQPMMPVSRPMGPVP
NPQPIDPASLEEFKRKILESQRPPAGIPVAPSSG

| | | |
|---|---|---|
| NOV27f, CG55193-02 | SEQ ID NO: 627 | 1367 bp |
| DNA Sequence | ORF Start: ATG at 1 | ORF Stop: TGA at 1327 |

ATGGGTCTGGCCATGGAGCACGGAGGGTCCTACGCTCGGGCGGGGGGCAGCTCTCGGGGCTGCTGGTA
TTACCTGCGCTACTTCTTCCTCTTCGTCTCCCTCATCCAATTCCTCATCATCCTGGGGCTCGTGCTCT
TCATGGTCTATGGCAACGTGCACGTGAGCACAGAGTCCAACCTGCAGGCCACCGAGCGCCGAGCCGAG
GGCCTATACAGTCAGCTCCTAGGGCTCACGGCCTCCCAGTCCAACTTGACCAAGGAGCTCAACTTCAC
CACCCGCGCCAAGGATGCCATCATGCAGATGTGGCTGAATGCTCGCCGCGACCTGGACCGCATCAATG
CCAGCTTCCGCCAGTGCCAGGGTGACCGGGTAATCTACACGAACAATCAGAGGTACATGGCTGCCATC
ATCTTGAGTGAGAAGCAATGCAGAGATCAATTCAAGGACATGAACAAGAGCTGCGATGCCTTGCTCTT
CATGCTGAATCAGAAGGTGAAGACGCTGGAGGTGGAGATAGCCAAGGAGAAGACCATTTGCACTAAGG
ATAAGGAAAGCGTGCTGCTGAACAAACGCGTGGCGGAGGAACAGCTGGTTGAATGCGTGAAAACCCGG
GAGCTGCAGCACCAAGAGCGCCAGCTGGCCAAGGAGCAACTGCAAAAGGTGCAAGCCCTCTGCCTGCC
CCTGGACAAGGACAAGTTTGAGATGGACCTTCGTAACCTGTGGAGGGACTCCATTATCCCACGCAGCC
TGGACAACCTGGGTTACAACCTCTACCATCCCCTGGGCTCGGAATTGGCCTCCATCCGCAGAGCCTGC
GACCACATGCCCAGCCTCATGAGCTCCAAGGTGGAAGGTCAGTGCCGGAGCCTCCGGGCGGATATCGA
ACGCGTGGCCCGCGAGAACTCAGACCTCCAACGCCAGAAGCTGGAAGCCCAGCAGGGCCTGCGGGCCA
GTCAGGAGGCGAAACAGAAGGTGGAGAAGGAGGCTCAGGCCCGGGAGGCCAAGCTCCAAGCTGAATGC
TCCCGGCAGACCCAGCTAGCGCTGGAGGAGAAGGCGGTGCTGCGGAAGGAACGAGACAACCTGGCCAA
GGAGCTGGAAGAGAAGAAGAGGGAGGCGGAGCAGCTCAGGATGGAGCTGGCCATCAGAAACTCAGCCC
TGGACACCTGCATCAAGACCAAGTCGCAGCCGATGATGCCAGTGTCAAGGCCCATGGGCCCTGTCCCC
AACCCCCAGCCCATCGACCCAGCTAGCCTGGAGGAGTTCAAGAGGAAGATCCTGGAGTCCCAGAGGCC
CCCTGCAGGCATCCCTGTAGCCCCATCCAGTGGCTGAGGAGGCTCCGGCACTGACCTAAGGGCGAATC
CCAGCAA

| | | | |
|---|---|---|---|
| NOV27f, CG55193-02 | SEQ ID NO: 628 | 442 aa | MW at 50568.7kD |
| Protein Sequence | | | |

MGLAMEHGGSYAPAGGSSRGCWYYLRYFFLFVSLIQFLIILGLVLFMVYGNVHVSTESNLQATERRAE
GLYSQLLGLTASQSNLTKELNFTTRAKDAIMQMWLNARRDLDRINASFRQCQGDRVIYTNNQRYMAAI
ILSEKQCRDQFKDMNKSCDALLFMLNQKVKTLEVEIAKEKTICTKDKESVLLNKRVAEEQLVECVKTR
ELQHQERQLAKEQLQKVQALCLPLDKDKFEMDLRNLWRDSIIPRSLDNLGYNLYHPLGSELASIRRAC
DHMPSLMSSKVEGQCRSLRADIERVARENSDLQRQKLEAQQGLRASQEAKQKVEKEAQAREAKLQAEC
SRQTQLALEEKAVLRKERDNLAKELEEKKREAEQLRMELAIRNSALDTCIKTKSQPMMPVSRPMGPVP
NPQPIDPASLEEFKRKILESQRPPAGIPVAPSSG

| | | |
|---|---|---|
| NOV27g, CG55193-03 | SEQ ID NO: 629 | 1367 bp |
| DNA Sequence | ORF Start: ATG at 1 | ORF Stop: TGA at 1327 |

ATGGGTCTGGCCATGGAGCACGGAGGGTCCTACGCTCGGGCGGGGGGCAGCTCTCGGGGCTGCTGGTA
TTACCTGCGCTACTTCTTCCTCTTCGTCTCCCTCATCCAATTCCTCATCATCCTGGGGCTCGTGCTCT
TCATGGTCTATGGCAACGTGCACGTGAGCACAGAGTCCAACCTGCAGGCCACCGAGCGCCGAGCCGAG
GGCCTATACAGTCAGCTCCTAGGGCTCACGGCCTCCCAGTCCAACTTGACCAAGGAGCTCAACTTCAC
CACCCGCGCCAAGGATGCCATCATGCAGATGTGGCTGAATGCTCGCCGCGACCTGGACCGCATCAATG
CCAGCTTCCGCCAGTGCCAGGGTGACCGGGTAATCTACACGAACAATCAGAGGTACATGGCTGCCATC
ATCTTGAGTGAGAAGCAATGCAGAGATCAATTCAAGGACATGAACAAGAGCTGCGATGCCTTGCTCTT
CATGCTGAATCAGAAGGTGAAGACGCTGGAGGTGGAGATAGCCAAGGAGAAGACCATTTGCACTAAGG
ATAAGGAAAGCGTGCTGCTGAACAAACGCGTGGCGGAGGAACAGCTGGTTGAATGCGTGAAAACCCGG
GAGCTGCAGCACCAAGAGCGCCAGCTGGCCAAGGAGCAACTGCAAAAGGTGCAAGCCCTCTGCCTGCC
CCTGGACAAGGACAAGTTTGAGATGGACCTTCGTAACCTGTGGAGGGACTCCATTATCCCACGCAGCC
TGGACAACCTGGGTTACAACCTCTACCATCCCCTGGGCTCGGAATTGGCCTCCATCCGCAGAGCCTGC
GACCACATGCCCAGCCTCATGAGCTCCAAGGTGGAAGGTCAGTGCCGGAGCCTCCGGGCGGATATCGA
ACGCGTGGCCCGCGAGAACTCAGACCTCCAACGCCAGAAGCTGGAAGCCCAGCAGGGCCTGCGGGCCA
GTCAGGAGGCGAAACAGAAGGTGGAGAAGGAGGCTCAGGCCCGGGAGGCCAAGCTCCAAGCTGAATGC
TCCCGGCAGACCCAGCTAGCGCTGGAGGAGAAGGCGGTGCTGCGGAAGGAACGAGACAACCTGGCCAA
GGAGCTGGAAGAGAAGAAGAGGGAGGCGGAGCAGCTCAGGATGGAGCTGGCCATCAGAAACTCAGCCC
TGGACACCTGCATCAAGACCAAGTCGCAGCCGATGATGCCAGTGTCAAGGCCCATGGGCCCTGTCCCC
AACCCCCAGCCCATCGACCCAGCTAGCCTGGAGGAGTTCAAGAGGAAGATCCTGGAGTCCCAGAGGCC
CCCTGCAGGCATCCCTGTAGCCCCATCCAGTGGCTGAGGAGGCTCCGGCACTGACCTAAGGGCGAATC
CCAGCAA

| | | | |
|---|---|---|---|
| NOV27g, CG55193-03 | SEQ ID NO: 630 | 442 aa | MW at 50568.7kD |
| Protein Sequence | | | |

MGLAMEHGGSYARAGGSSRGCWYYLRYFFLFVSLIQFLIILGLVLFMVYGNVHVSTESNLQATERRAE
GLYSQLLGLTASQSNLTKELNFTTRAKDAIMQMWLNARRDLDRINASFRQCQGDRVIYTNNQRYMAAI
ILSEKQCRDQFKDMNKSCDALLFMLNQKVKTLEVEIAKEKTICTKDKESVLLNKRVAEEQLVECVKTR
ELQHQERQLAKEQLQKVQALCLPLDKDKFEMDLRNLWRDSIIPRSLDNLGYNLYHPLGSELASIRRAC
DHMPSLMSSKVEGQCRSLRADIERVARENSDLQRQKLEAQQGLRASQEAKQKVEKEAQAREAKLQAEC
SRQTQLALEEKAVLRKERDNLAKELEEKKREAEQLRMELAIRNSALDTCIKTKSQPMMPVSRPMGPVP
NPQPIDPASLEEFKRKILESQRPPAGIPVAPSSG

| | | |
|---|---|---|
| NOV27h, CG55193-05 | SEQ ID NO: 631 | 1188 bp |
| DNA Sequence | ORF Start: at 7 | ORF Stop: at 1183 |

GGATCCAACGTGCACGTGAGCACAGAGTCCAACCTGCAGGCCACCGAGCACCGAGCCGAGGGCCTATA
CAGTCAGCTCCTAGGGCTCACGGCCTCCCAGTCCAACTTGACCAAGGAGCTCAACTTCACCACCCGCG
CCAAGGATGCCATCATGCAGATGTGGCTGAATGCTCGCCGCGACCTGGACCGCATCAATGCCAGCTTC
CGCCAGTGCCAGGGTGACCGGGTCATCTACACGAACAATCAGAGGTACATGGCTGCCATCATCTTGAG
TGAGAAGCAATGCAGAGATCAATTCAAGGACATGAACAAGAGCTGCGATGCCTTGCTCTTCATGCTGA
ATCAGAAGGTGAAGACGCTGGAGGTGGAGATAGCCAAGGAGAAGACCATTTGCACTAAGGATAAGGAA
AGCGTGCTGCTGAACALACGCGTGGCGGAGGAACAGCTGGTTGAATGCGTGAAAACCCGGGAGCTGCA
GCACCAAGAGCGCCAGCTGGTCAAGGAGCAACTGCAAAAGGTGCAAGCCCTCTGCCTGCCCCTGGACA
AGGACAAGTTTGAGATGGACCTTCGTAACCTGTGGAGGGACTCCATTATCCCACGCAGCCTGGACAAC
CTGGGTTACAACCTCTACCATCCCCTGGGCTCGGAATTGGCCTCCATCCGCAGAGCCTGCGACCACAT
GCCCAGCCTCATGAGCTCCAAGGTGGAGGAGCTGGCCCGGAGCCTCCGGGCGGATATCGAACGCGTGG
TCCGCGAGAACTCAGACCTCCAACGCCAGAAGCTGGAAGCCCAGCAGGGCCTGCGGGCCAGTCAGGAG
GCGAAACAGAAGGTGGAGAAGGAGGCTCAGGCCCGGGAGGCCAAGCTCCAAGCTGAATGCTCCCGGCA
GACCCAGCTAGCGCTGGAGGAGAAGGCGGTGCTGCGGAGGGAACGAGACAACCTGGCCAAGGAGCTGG
AAGAGAAGAAGAGGGAGGCGGAGCAGCTCAGGATGGAGCTGGCCATCAGAAACTCAGCCCTGGACACC
TGCATCAAGACCAAGTCGCAGCCGATGATGCCAGTGTCAAGGCCCATGGGCCCTGTCCCCAACCCCCA
GCCCATCGACCCAGCTAGCCTGGAGGAGTTCAAGAGGAAGATCCTGGAGTCCCAGAGGCCCCCTGCAG
GCATCCCTGTAGCCCCATCCAGTGGCCTCGAG

| | | | |
|---|---|---|---|
| NOV27h, CG55193-05 | SEQ ID NO: 632 | 392 aa | MW at 45001.1kD |
| Protein Sequence | | | |

NVHVSTESNLQATEHRAEGLYSQLLGLTASQSNLTKELNFTTRAKDAIMQMWLNARRDLDRINASFRQ
CQGDRVIYTNNQRYMAAIILSEKQCRDQFKDMNKSCDALLFMLNQKVKTLEVEIAKEKTICTKDKESV
LLNKRVAEEQLVECVKTRELQHQERQLVKEQLQKVQALCLPLDKDKFEMDLRNLWRDSIIPRSLDNLG
YNLYHPLGSELASIRRACDHMPSLMSSKVEELARSLRADIERVVRENSDLQRQKLEAQQGLRASQEAK
QKVEKEAQAREAKLQAECSRQTQLALEEKAVLRRERDNLAKELEEKKREAEQLRMELAIRNSALDTCI
KTKSQPMMPVSRPMGPVPNPQPIDPASLEEFKRKILESQRPPAGIPVAPSSG

| | | |
|---|---|---|
| NOV27i, CG55193-06 | SEQ ID NO: 633 | 1188 bp |
| DNA Sequence | ORF Start: at 7 | ORF Stop: at 1183 |

GGATCCAACGTGCACGTGAGCACAGAGTCCAACCTGCAGGCCACCGAGCGCCGAGCCGAGGGCCTATA
CAGTCAGCTCCTAGGGCTCACGGCCTCCCAGTCCAACTTGACCAAGGAGCTCAACTTCACCACCCGCG
CCAAGGATGCCATCATGCAGATGTGGCTGAATGCTCGCCGCGACCTGGACCGCATCAATGCCAGCTTC
CGCCAGTGCCAGGGTGACCGGGTCATCTACACGAACAATCAGAGGTACATGGCTGCCATCATCTTGAG
TGAGAAGCAATGCAGAGATCAATTCAAGGACATGAACAAGAGCTGCGATGCCTTGCTCTTCATGCTGA
ATCAGAAGGTGAAGACGCTGGAGGTGGAGATAGCCAAGGAGAAGACCATTTGCACTAAGGATAAGGAA
AGCGTGCTGCTGAACAAACGCGTGGCGGAGGAACAGCTGGTTGAATGCGTGAAAACCCGGGAGCTGCA
GCACCAAGAGCGCCAGCTGGCCAAGGAGCAACTGCAAAAGGTGCAAGCCCTCTGCCTGCCCCTGGACA
AGGACAAGTTTGAGATGGACCTTCGTAACCTGTGGAGGGACTCCATTATCCCACGCAGCCTGGACAAC
CTGGGTTACAACCTCTACCATCCCCTGGGCTCGGAATTGGCCTCCATCCGCAGAGCCTGCGACCACAT
GCCCAGCCTCATGAGCTCCAAGGTGGAGGAGCTGGCCCGGAGCCTCCGGGCGGATATCGAACGCGTGG
CCCGCGAGAACTCAGACCTCCAACGCCAGAAGCTGGAAGCCCAGCAGGGCCTGCGGGCCAGTCAGGAG
GCGAAACAGAAGGTGGAGAAGGAGGCTCAGGCCCGGGAGGCCAAGCTCCAAGCTGAATGCTCCCGGCA
GACCCAGCTAGCGCTGGAGGAGAAGGCGGTGCTGCGGAAGGAACGAGACAACCTGGCCAAGGAGCTGG
AAGAGAAGAAGAGGGAGGCGGAGCAGCTCAGGATGGAGCTGGCCATCAGAAACTCAGCCCTGGACACC
TGCATCAAGACCAAGTCGCAGCCGATGATGCCAGTGTCAAGGCCCATGGGCCCTGTCCCCAACCCCCA
GCCCATCGACCCAGCTAGCCTGGAGGAGTTCAAGAGGAAGATCCTGGAGTCCCAGAGGCCCCCTGCAG
GCATCCCTGTAGCCCCATCCAGTGGCCTCGAG

| | | | |
|---|---|---|---|
| NOV27i, CG55193-06 | SEQ ID NO: 634 | 392 aa | MW at 44936.0kD |
| Protein Sequence | | | |

NVHVSTESNLQATERRAEGLYSQLLGLTASQSNLTKELNFTTEAKDAIMQMWLNARRDLDRINASFRQ
CQGDRVIYTNNQRYMAAIILSEKQCRDQFKDMNKSCDALLFMLNQKVKTLEVEIAKEKTICTKDKESV
LLNKRVAEEQLVECVKTRELQHQERQLAKEQLQKVQALCLPLDKDKFEMDLRNLWRDSIIPRSLDNLG
YNLYHPLGSELASIRRACDHMPSLMSSKVEELARSLRADIERVARENSDLQRQKLEAQQGLRASQEAK
QKVEKEAQAREAKLQAECSRQTQLALEEKAVLRKERDNLAKELEEKKREAEQLRMELAIRNSALDTCI
KTKSQPMNPVSRPMGPVPNPQPIDPASLEEFKRKILESQRPPAGIPVAPSSG

| | | |
|---|---|---|
| NOV27j, CG55193-07 | SEQ ID NO: 635 | 1410 bp |
| DNA Sequence | ORF Start: ATG at 79 | ORF Stop: at 1405 |

GAGATTCCCCCCCCTCGGGTCTCTTGGGCCTGTCCATGCGCCCCCCCCTCCGTGCTCCATGGCCAGAC
CCATGGATCCATGGGTCTGGCCATGGAGCACGGAGGGTCCTACGCTCGGGCGGGGGGCAGCTCTCGGG
GCTGCTGGTATTACCTGCGCTACTTCTTCCTCTTCGTCTCCCTCATCCAATTCCTCATCATCCTGGGG
CTCGTGCTCTTCATGGTCTATGGCAACGTGCACGTGAGCACAGAGTCCAACCTGCAGGCCACCGAGCG
CCGAGCCGAGGGCCTATACAGTCAGCTCCTAGGGCTCACGGCCTCCCAGTCCAACTTGACCAAGGAGC
TCAACTTCACCACCCGCGCCAAGGATGCCATCATGCAGATGTGGCTGAATGCTCGCcGCGACCTGGAC
CGCATCAATGCCAGCTTCCGCCAGTGCCAGGGTGACCGGGTCATCTACACGAACAATCAGAGGTACAT
GGCTGCCATCATCTTGAGTGAGAAGCAATGCAGAGATCAATTCAAGGACATGAACAAGAGCTGCGATG
CCTTGCTCTTCATGCTGAATCAGAAGGTGAAGACGCTGGAGGTGGAGATAGCCAAGGAGAAGACCATT
TGCACTAAGGATAAGGAAAGCGTGCTGCTGAACAAACGCGTGGCGGAGGAACAGCTGGTTGAATGCGT
GAAAACCCGGGAGCTGCAGCACCAAGAGCGCCAGCTGGCCAAGGAGCAACTGCAAAAGGTGCAAGCCC
TCTGCCTGCCCCTGGACAAGGACAAGTTTGAGATGGACCTTCGTAACCTGTGGAGGGACTCCATTATC
CCACGCAGCCTGGACAACCTGGGTTACAACCTCTACCATCCCCTGGGCTCGGAATTGGCCTCCATCCG
CAGAGCCTGCGACCACATGCCCAGCCTCATGAGCTCCAAGGTGGAGGAGCTGGCCCGGAGCCTCCGGG
CGGATATCGAACGCGTGGCCCGCGAGAACTCAGACCTCCAACGCCAGAAGCTGGAAGCCCAGCAGGGC
CTGCGGGCCAGTCAGGAGGCGAAACAGAAGGTGGAGAAGGAGGCTCAGGCCCGGGAGGCCAAGCTCCA
AGCTGAATGCTCCCGGCAGACCCAGCTAGCGCTGGAGGAGAAGGCGGTGCTGCGGAAGGAACGAGACA
ACCTGGCCAAGGAGCTGGAAGAGAAGAAGAGGGAGGCGGAGCAGCTCAGGATGGAGCTGGCCATCAGA
AACTCAGCCCTGGACACCTGCATCAAGACCAAGTCGCAGCCGATGATGCCAGTGTCAAGGCCCATGGG
CCCTGTCCCCAACCCCCAGCCCATCGACCCAGCTAGCCTGGAGGAGTTCAAGAGGAAGATCCTGGAGT
CCCAGAGGCCCCCTGCAGGCATCCCTGTAGCCCCATCCAGTGGCCTCGAG

| | | | |
|---|---|---|---|
| NOV27j, CG55193-07 | SEQ ID NO: 636 | 442 aa | MW at 50593.7kD |
| Protein Sequence | | | |

MGLAMEHGGSYARAGGSSRGCWYYLRYFFLFVSLIQFLIILGLVLFMVYGNVHVSTESNLQATERRAE
GLYSQLLGLTASQSNLTKELNFTTRAKDAIMQMWLNARRDLDRINASFRQCQGDRVIYTNNQRYMAAI
ILSEKQCRDQFKDMNKSCDALLFMLNQKVKTLEVEIAKEKTICTKDKESvLLNKRvAEEQLVECVKTR
ELQHQERQLAKEQLQKVQALCLPLDKDKFEMDLRNLWRDSIIPRSLDNLGYNLYHPLGSELASIRRAC
DHMPSLMSSKVEELARSLRADIERVARENSDLQRQKLEAQQGLRASQEAKQKVEKEAQAREAKLQAEC
SRQTQLALEEKAVLRKERDNLAKELEEKKREAEQLRMELAIRNSALDTCIKTKSQPMMPVSRPMGPVP
NPQPIDPASLEEFKRKTLESQRPPAGIPVAPSSG

A ClustalW comparison of the above protein sequences yields the following sequence alignment shown in Table 27B.

TABLE 27B
Comparison of the NOV27 protein sequences.

NOV27a --------------------------MGLAMEHGGSYARAGGSSRGCWYYLRYFFLFVSL
NOV27b ------------------------------------------------------------
NOV27c EIPPPRVSWACPCAPPSVLHGQTHGSMGLAMEHGGSYARAGGSSRGCWYYLRYFFLFVSL
NOV27d ---------------------FALGSMGLAMEHGGSYARAGGSSRGCWYYLRYFFLFVSL
NOV27e --------------------------MGLAMEHGGSYARAGGSSRGCWYYLRYFFLFVSL
NOV27f --------------------------MGLAMEHGGSYAHAGGSSRGCWYYLRYFFLFVSL
NOV27g --------------------------MGLAMEHGGSYARAGGSSRGCWYYLRYFFLFVSL
NOV27h ------------------------------------------------------------
NOV27i ------------------------------------------------------------
NOV27j --------------------------MGLAMEHGGSYARAGGSSRGCWYYLRYFFLFVSL
NOV27a IQFLIILGLVLFMVYGNVHVSTESNLQATERRAEGLYSQLLGLTASQSNLTKELNFTTRA
NOV27b --------------GSNVHVSTESNLQATEHRAEGLYSQLLGLTASQSNLTKELNFTTRA
NOV27c IQFLIILGLVLFMVYGNVHVSTESNLQATERRAEGLYSQLLGLTASQSNLTKELNFTTRA
NOV27d IQFLIILGLVLFMVYGNVHVSTESNLQATERRAEGLYSQLLGLTASQSNLTKELNFTTRA
NOV27e IQFLIILGLVLFMVYGNVHVSTESNLQATERRAEGLYSQLLGLTASQSNLTKELNFTTRA
NOV27f IQFLIILGLVLFMVYGNVHVSTESNLQATERRAEGLYSQLLGLTASQSNLTKELNFTTRA
NOV27g IQFLIILGLVLFMVYGNVHVSTESNLQATERRAEGLYSQLLGLTASQSNLTKELNFTTRA
NOV27h ----------------NVHVSTESNLQATEHRAEGLYSQLLGLTASQSNLTKELNFTTRA
NOV27i ----------------NVHVSTESNLQATERRAEGLYSQLLGLTASQSNLTKELNFTTRA
NOV27j IQFLIILGLVLFMVYGNVHVSTESNLQATERRAEGLYSQLLGLTASQSNLTKELNFTTRA
NOV27a KDAIMQMWLNARRDLDRINASFRQCQGDRVIYTNNQRYMAAIILSEKQCRDQFKDMNKSC
NOV27b KDAIMQMWLNARRDLDRINASFRQCQGDRVIYTNNQRYMAAIILSEKQCRDQFKDMNKSC
NOV27c KDAIMQMWLNAREDLDRINASFRQCQGDRVIYTNNQRYMAAIILSEKQCRDQFKDMNKSC
NOV27d KDAIMQMWLNARRDLDRINASFRQCQGDRVIYTNNQRYMAAIILSEKQCRDQFKDMNKSC
NOV27e KDAIMQMWLNARPDLDRINASFRQCQGDRVIYTNNQRYMAAIILSEKQCRDQFKDMNKSC
NOV27f KDAIMQMWLNARRDLDRINASFRQCQGDRVIYTNNQRYMAAIILSEKQCRDQFKDMNKSC
NOV27g KDAIMQMWLNARRDLDRINASFRQCQGDRVIYTNNQRYMAAIILSEKQCRDQFKDMNKSC
NOV27h KDAIMQMWLNARRDLDRINASFRQCQGDRVIYTNNQRYNAAIILSEKQCRDQFKDMNKSC
NOV27i KDAIMQMWLNARRDLDRINASFRQCQGDRVIYTNNQRYMAAIILSEKQCRDQFKDMNKSC
NOV27j KDAIMQMWLNARPDLDRINASFRQCQGDRVIYTNNQRYMAAIILSEKQCRDQFKDMNKSC
NOV27a DALLFMLNQKVKTLEVEIAKEKTICTKDKESVLLNKRVAEEQLVECVKTRELQHQERQLA
NOV27b DALLFMLNQKVKTLEVEIAKEKTICTKDKESVLLNKRVAEEQLVECVKTRELQHQERQLV
NOV27c DALLFMLNQKVKTLEVEIAKEKTICTKDKESVLLNKRVAEEQLVECVKTRELQHQERQLA
NOV27d DALLFMLNQKVKTLEVEIAKEKTICTKDKESVLLNKRVAEEQLVECVKTRELRHQERQLA
NOV27e DALLFMLNQKVKTLEVEIAKEKTICTKDKESVLLNKRVAEEQLVECVKTRELQHQERQLA
NOV27f DALLFMLNQKVKTLEVEIAKEKTICTKDKESVLLNKRVAEEQLVECVKTRELQHQERQLA
NOV27g DALLFMLNQKVKTLEVEIAKEKTICTKDKESVLLNKRVAEEQLVECVKTRELQHQERQLA
NOV27h DALLFMLNQKVKTLEVEIAKEKTICTKDKESVLLNKRVAEEQLVECVKTRELQHQERQLV
NOV27i DALLFMLNQKVKTLEVEIAKEKTICTKDKESVLLNKRVAEEQLVECVKTRELQHQERQLA
NOV27j DALLFMLNQKVKTLEVEIAKEKTICTKDKESVLLNKRVAEEQLVECVKTRELQHQERQLA
NOV27a KEQLQKVQALCLPLDKDKFEMDLRNLWRDSIIPRSLDNLGYNLYHPLGSELASIRRACDH
NOV27b KEQLQKVQALCLPLDKDKFEMDLRNLWRDSIIPRSLDNLGYNLYHPLGSELASIRRACDH
NOV27c KEQLQKVQALCLPLDKDKFEMDLRNLWRDSIIPRSLDNLGYNLYHPLGSELASIRRACDH
NOV27d KEQLQKVQALCLPLDKDKFEMDLRNLWRDSIIPRSLDNLGYNLYHPLGSELASIRRACDH
NOV27e KEQLQKVQALCLPLDKDKFEMDLRNLWRDSIIPRSLDNLGYNLYHPLGSELASIRPACDH
NOV27f KEQLQKVQALCLPLDKDKFEMDLRNLWRDSIIPRSLDNLGYNLYHPLGSELASIRRACDH
NOV27g KEQLQKVQALCLPLDKDKFEMDLRNLWRDSIIPRSLDNLGYNLYHPLGSELASIRPACDH
NOV27h KEQLQKVQALCLPLDKDKFEMDLRNLWRDSIIPRSLDNLGYNLYHPLGSELASIRPACDH
NOV27i KEQLQKVQALCLPLDKDKFEMDLRNLWRDSIIPRSLDNLGYNLYHPLGSELASIRPACDH
NOV27j KEQLQKVQALCLPLDKDKFEMDLRNLWRDSIIPRSLDNLGYNLYHPLGSELASIRRACDH
NOV27a MPSLMSSKVEGQCRSLRADIERVAPENSDLQRQKLEAQQGLEASQEAKQKVEKEAQAREA
NOV27b MPSLMSSKVEELARSLRADIERVVRENSDLQRQKLEAQQGLRASQEAKQKVEKEAQAREA
NOV27c MPSLMSSKVEELARSLRADIERVARENSDLQRQKLEAQQGLRASQEAKQKVEKEAQAREA
NOV27d MPSLMSSKVEELARSLRADIERVARENSDLQRQKLEAQQGLEASQEAKQKVEKEAQAREA
NOV27e MPSLMSSKVEGQCRSLRADIERVARENSDLQRQKLEAQQGLEASQEAKQKVEKEAQAREA
NOV27f MPSLMSSKVEGQCRSLRADIERVARENSDLQRQKLEAQQGLPASQEAKQKVEKEAQAREA
NOV27g MPSLMSSKVEGQCRSLRADIERVARENSDLQRQKLEAQQGLPASQEAKQKVEKEAQAREA
NOV27h MPSLMSSKVEELARSLHADIERVVRENSDLQRQKLEAQQGLEASQEAKQKVEKEAQAREA
NOV27i MPSLMSSKVEELARSLRADIERVARENSDLQRQKLEAQQGLRASQEAKQKVEKEAQAREA
NOV27j MPSLMSSKVEELARSLEADIERVARENSDLQRQKLEAQQGLPASQEAKQKVEKEAQAREA
NOV27a KLQAECSRQTQLALEEKAVLRKERDNLAKELEEKKREAEQLRMETAIRNSALDTCIKTKS
NOV27b KLQAECSRQTQLALEEKAVLRRERDNLAKELEEKKREAEQLRMELAIRNSALDTCIKTKS
NOV27c KLQAECSRQTQLALEEKAVLRKERDNLAKELEEKKREAEQLRMELAIRNSALDTCIKTKS
NOV27d KLQAECSRQTQLALEEKAVLRKERDNLAKELEEKKREAEQLRMELAIRNSALDTCIKTKS
NOV27e KLQAECSRQTQLALEEKAVLRKERDNLAKELEEKKREAEQLPMELAIRNSALDTCIKTKS
NOV27f KLQAECSRQTQLALEEKAVLRKERDNLAKELEEKKREAEQLRMELAIRNSALDTCIKTKS
NOV27g KLQAECSRQTQLALEEKAVLRKERDNLAKELEEKKREAEQLRNELAIRNSALDTCIKTKS
NOV27h KLQAECSRQTQLALEEKAVLRRERDNLAKELEEKKREAEQLRMELAIRNSALDTCIKTKS
NOV27i KLQAECSRQTQLALEEKAVLRKERDNLAKELEEKKREAEQLRMELAIRNSALDTCIKTKS
NOV27j KLQAECSRQTQLALEEKAVLRKERDNLAKELEEKKREAEQLRMELAIRNSALDTCIKTKS
NOV27a QPMMPVSRPMGPVPNPQPIDPASLEEFKRKILESQRPPAGIPVAPSSG------------
NOV27b QPMMPVSRPMGPVPNPQPIDPASLEEFKRKILESQRPPAGIPVAPSSGLE----------
NOV27c QPMMPVSRPMGPVPNPQPIDPASLEEFKRKILESQRPPAGIPVAPSSGLE----------
NOV27d QPMMPVSRPMGPVPNPQPIDPASLEEFKRKILESQRPPAGIPVAPSSGLEATGWGYRDAS
NOV27e QPMMPVSRPMGPVPNPQPIDPASLEEFKRKILESQRPPAGIPVAPSSG------------
NOV27f QPMMPVSRPMGPVPNPQPIDPASLEEFKRKILESQRPPAGIPVAPSSG------------
NOV27g QPMMPVSRPMGPVPNPQPIDPASLEEFKRKILESQRPPAGIPVAPSSG------------
NOV27h QPMMPVSRPMGPVPNPQPIDPASLEEFKRKILESQRPPAGIPVAPSSG------------
NOV27i QPMMPVSRPMGPVPNPQPIDPASLEEFKRKILESQRPPAGIPVAPSSG------------
NOV27j QPMMPVSRPMGPVPNPQPIDPASLEEFKRKILESQRPPAGIPVAPSSG------------
NOV27a ---------
NOV27b ---------
NOV27c ---------
NOV27d VLHGQTHGS
NOV27e ---------
NOV27f ---------
NOV27g ---------
NOV27h ---------
NOV27i ---------
NOV27j ---------
NOV27a (SEQ ID NO: 618)
NOV27b (SEQ ID NO: 620)
NOV27c (SEQ ID NO: 622)
NOV27d (SEQ ID NO: 624)
NOV27e (SEQ ID NO: 626)
NOV27f (SEQ ID NO: 628)
NOV27g (SEQ ID NO: 630)
NOV27h (SEQ ID NO: 632)
NOV27i (SEQ ID NO: 634)
NOV27j (SEQ ID NO: 636)

Further analysis of the NOV27a protein yielded the following properties shown in Table 27C.

TABLE 27C
Protein Sequence Properties NOV27a

| | |
|---|---|
| SignalP analysis: | Cleavage site between residues 51 and 52 |
| PSORT II analysis: | |

| PSG: | a new signal peptide prediction method | |
|---|---|---|
| | N-region: | length 6; pos. chg 0; neg. chg 1 |
| | H-region: | length 6; peak value 0.00 |
| | PSG score: | −4.40 |

| | |
|---|---|
| GvH: | von Heijne's method for signal seq. recognition |
| | GvH score (threshold: −2.1): −1.10 |
| | possible cleavage site: between 50 and 51 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 1 | | |
| | Number of TMS(s) for threshold 0.5: 1 | | |
| | INTEGRAL | Likelihood = | Transmembrane 29-25 |
| | | −11.41 | |

| | |
|---|---|
| PERIPHERAL | Likelihood = 7.85 (at 156) |

| | |
|---|---|
| | ALOM score: −11.41 (number of TMSs: 1) |
| MTOP: | Prediction of membrane topology (Hartmann et al.) |
| | Center position for calculation: 36 |
| | Charge difference: −2.5 C(0.5)-N(3.0) |
| | N >= C: N-terminal side will be inside |

>>> membrane topology: type 2 (cytoplasmic tail 1 to 29)

| MITDISC: | discrimination of mitochondrial targeting seq | | | |
|---|---|---|---|---|
| | R content: | 3 | Hyd Moment(75): | 5.06 |
| | Hyd Moment (95): | 6.63 | G content: | 8 |
| | D/E content: | 2 | S/T content: | 6 |

| | |
|---|---|
| | Score: −6.39 |
| Gavel: | prediction of cleavage sites for mitochondrial preseq |
| | R-2 motif at 36 LRY\|FF |

NUCDISC: discrimination of nuclear localization signals

| | |
|---|---|
| | pat4: none |
| | pat7: none |
| | bipartite: RKERDNLAKELEEKKRE at 355 |
| | content of basic residues: 15.6% |
| | NLS Score: 0.02 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: 10, 22, 23, 26
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 89 |
| COIL: | Lupas's algorithm to detect coiled-coil regions |
| 278 M | 0.60 |
| 279 S | 0.85 |
| 280 S | 0.96 |
| 281 K | 0.96 |
| 282 V | 0.96 |
| 283 E | 0.98 |
| 284 G | 0.98 |
| 285 Q | 0.99 |
| 286 C | 0.99 |
| 287 R | 0.99 |
| 288 S | 0.99 |
| 289 L | 0.99 |
| 290 R | 0.99 |
| 291 A | 0.99 |
| 292 D | 0.99 |
| 293 I | 0.99 |
| 294 E | 0.99 |
| 295 R | 0.99 |
| 296 V | 0.99 |
| 297 A | 0.99 |
| 298 R | 0.99 |
| 299 E | 0.99 |
| 300 N | 0.99 |
| 301 S | 0.99 |
| 302 D | 1.00 |
| 303 L | 1.00 |
| 304 Q | 1.00 |
| 305 R | 1.00 |
| 306 Q | 1.00 |
| 307 K | 1.00 |
| 308 L | 1.00 |
| 309 E | 1.00 |
| 310 A | 1.00 |
| 311 Q | 1.00 |
| 312 Q | 1.00 |
| 313 G | 1.00 |
| 314 L | 1.00 |
| 315 R | 1.00 |
| 316 A | 1.00 |
| 317 S | 1.00 |
| 318 Q | 1.00 |
| 319 E | 1.00 |
| 320 A | 1.00 |
| 321 K | 1.00 |
| 322 Q | 1.00 |
| 323 K | 1.00 |
| 324 V | 1.00 |
| 325 E | 1.00 |
| 326 K | 1.00 |
| 327 E | 1.00 |
| 328 A | 1.00 |
| 329 Q | 1.00 |
| 330 A | 0.99 |
| 331 R | 0.96 |
| 332 E | 0.96 |
| 333 A | 0.96 |
| 334 K | 0.96 |
| 335 L | 0.96 |
| 336 Q | 0.96 |
| 337 A | 0.87 |
| 338 E | 0.72 |
| 339 C | 0.71 |
| 340 S | 0.75 |
| 341 R | 0.92 |
| 342 Q | 0.92 |
| 343 T | 0.93 |
| 344 Q | 0.99 |
| 345 L | 1.00 |
| 346 A | 1.00 |
| 347 L | 1.00 |
| 348 E | 1.00 |
| 349 E | 1.00 |
| 350 K | 1.00 |
| 351 A | 1.00 |
| 352 V | 1.00 |
| 353 L | 1.00 |
| 354 R | 1.00 |
| 355 K | 1.00 |
| 356 E | 1.00 |
| 357 R | 1.00 |
| 358 D | 1.00 |
| 359 N | 1.00 |
| 360 L | 1.00 |
| 361 A | 1.00 |
| 362 K | 1.00 |
| 363 E | 1.00 |
| 364 L | 1.00 |
| 365 E | 1.00 |
| 366 E | 1.00 |
| 367 K | 1.00 |
| 368 K | 1.00 |
| 369 R | 1.00 |
| 370 E | 1.00 |
| 371 A | 1.00 |
| 372 E | 1.00 |
| 373 Q | 1.00 |
| 374 L | 1.00 |
| 375 R | 1.00 |
| 376 M | 1.00 |
| 377 E | 1.00 |
| 378 L | 1.00 |
| 379 A | 1.00 |
| 380 I | 1.00 |
| 381 R | 0.97 |
| 382 N | 0.97 |
| 383 S | 0.97 |
| 384 A | 0.97 |
| 385 L | 0.97 |
| 386 D | 0.97 |
| 387 T | 0.91 |
| 388 C | 0.64 |
| 389 I | 0.64 |
| 390 K | 0.64 |
| | total: 113 residues |

Final Results (k = 9/23):

```
39.1%: nuclear
26.1%: mitochondrial
17.4%: cytoplasmic
  4.3%: extracellular, including cell wall
  4.3%: Golgi
  4.3%: plasma membrane
  4.3%: peroxisomal
```

>> prediction for CG55193-04 is nuc (k = 23)

A search of the NOV27a protein against the Geneseq database, a proprietary se that contains sequences published in patents and patent publication, yielded homologous proteins shown in Table 27D.

TABLE 27D
Geneseq Results for NOV27a

| | | NOV27a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABG78367 | Human protein, homologous to PV-1 | 1 . . . 442 | 442/442 (100%) | 0.0 |
| | receptor, designated NOV7a - Homo | 1 . . . 442 | 442/442 (100%) | |
| | sapiens, 442 aa. [WO200226826-A2, | | | |
| | 04-APR-2002] | | | |
| AAE06600 | Human protein having hydrophobic | 1 . . . 442 | 439/442 (99%) | 0.0 |
| | domain, HP10787 - Homo sapiens, 442 aa. | 1 . . . 442 | 439/442 (99%) | |
| | [WO200149728-A2, 12-JUL-2001] | | | |
| AAM41619 | Human polypeptide SEQ ID NO 6550 - | 1 . . . 442 | 439/442 (99%) | 0.0 |
| | Homo sapiens, 457 aa. | 16 . . . 457 | 439/442 (99%) | |
| | [WO200153312-A1, 26-JUL-2001] | | | |
| AAB42154 | Human ORFX ORF1918 polypeptide sequence | 1 . . . 442 | 439/442 (99%) | 0.0 |
| | SEQ ID NO: 3836 - Homo | 38 . . . 479 | 439/442 (99%) | |
| | sapiens, 479 aa. [WO200058473-A2, | | | |
| | 05-OCT-2000] | | | |
| ABG78369 | Human protein, homologous to PV-1 | 1 . . . 442 | 436/442 (98%) | 0.0 |
| | receptor, designated NOV7c - Homo | 1 . . . 442 | 439/442 (98%) | |
| | sapiens, 442 aa. [WO200226826-A2, | | | |
| | 04-APR-2002] | | | |

In a BLAST search of public sequence databases, the NOV27a protein was found to have homology to the proteins shown in the BLASTP data in Table 27E.

TABLE 27E
Public BLASTP Results for NOV27a

| | | NOV27a | Identities/ | |
|---|---|---|---|---|
| Protein | | Residues/ | Similarities for | |
| Accession | | Match | the Matched | Expect |
| Number | Protein/Organism/Length | Residues | Portion | Value |
| AAH50365 | PLVAP protein - Homo sapiens | 1 . . . 442 | 439/442 (99%) | 0.0 |
| | (Human), 455 aa (fragment). | 14 . . . 455 | 439/442 (99%) | |
| Q9BX97 | PV1 protein - Homo sapiens | 1 . . . 442 | 439/442 (99%) | 0.0 |
| | (Human), 442 aa. | 1 . . . 442 | 439/442 (99%) | |
| Q8ND68 | Hypothetical protein - Homo sapiens | 1 . . . 442 | 439/442 (99%) | 0.0 |
| | (Human), 456 aa (fragment). | 15 . . . 456 | 439/442 (99%) | |
| Q9BZD5 | Fenestrated-endothelial linked | 1 . . . 442 | 438/442 (99%) | 0.0 |
| | structure protein - Homo sapiens | 1 . . . 442 | 438/442 (99%) | |
| | (Human), 442 aa. | | | |
| Q8N8Y0 | Hypothetical protein FLJ38711 - | 1 . . . 442 | 433/442 (97%) | 0.0 |
| | Homo sapiens (Human), 437 aa. | 1 . . . 437 | 433/442 (97%) | |

PFam analysis predicts that the NOV27a protein contains the domains shown in the Table 27F.

TABLE 27F
Domain Analysis of NOV27a

| | | | |
|---|---|---|---|
| Pfam | NOV27a | Identities/ | Expect |
| Domain | Match Region | Similarities | Value |
| | | for the | |
| | | Matched Region | |

### Example 28

The NOV28 clone was analyzed, and the nucleotide and encoded polypeptide sequences are shown in Table 28A.

TABLE 28A
NOV28 Sequence Analysis

| | | |
|---|---|---|
| NOV28a, CG55256-07 | SEQ ID NO:637 | 4294 bp |
| DNA Sequence | ORF Start: ATG at 103 | ORF Stop: TGA at 2818 |

GCAGCGGCAACCCCGGCGCCGCGGCAAGGACTCGGAGGGCTGAGACGCGGCGGCGGCGGCGCGGGGAG
CGCGGGGCGCGGCGGCCGGAGCCCCGGGCCCGCCATGGGCCTCCCCGAGCCGGGCCCTCTCCGGCTTC
GATCCGCTGCTCGGCGGCCAAGGGCCGGCCAAGGAGTGCGAAAAGGACCAATTCCAGTGCCGGAACGA
GCGCTGCATCCCCTCTGTGTGGAGATGCGACGAGGACGATGACTGCTTAGACCACAGCGACGAGGACG
ACTGCCCCAAGAAGACCTGTGCAGACAGTGACTTCACCTGTGACAACGGCCACTGCATCCACGAACGG
TGGAAGTGTGACGGCGAGGAGGAGTGTCCTGATGGCTCCGATGAGTCCGAGGCCACTTGCACACTGGG
CACCTGCCGTGGGGACGAGTTCCAGTGTGGGGATGGGACATGTGTCCTTGCAATCAAGCACTGCAACC
AGGAGCAGGACTGTCCAGATGGGAGTGATGAAGCTGGCTGCCTACAGGAGTCACCCTGTGAGGGTCCC
CGCAGATTTCAGTGTAAGAGTGGCAAGCGCGTGGACGGCGGGAAAGTGTGTGATGTGCAGAGGGACTG
CCGGGACTGGTCGGATGAGCTTCTGAAAGTGTGGTGCGGTGCCTGTCTACGCCCACTGGCTGGACTCA
GTCTCCTACCATCCCCCTCCTGGTATCTAGGCTCAAGGCCCTCCAGTGCCCCCTGCCCTGACACTTTC
TGCTCTGACCCTCTCTTTGGATTCATGTGCCGTCCTATGGCTTCACATGGGGCTTTTCGCCCCCAGGC
CTCTGGTCTACATCTCTACAAAGTGCTAAGAGCTTGTCCATCCCAGAGGAGGCTGAACGAGTGTCTGC
ACAACAATGGCGGCTGCTCACACATCTGCACTGACCTCAAGATTGGCTTTGAATGCACGTGCCCAGCA
GGCTTCCAGCTCCTGGACCAGAAGACCTGTGGCGACATTGATGAGTGCAAGGACCCAGATGCCTGCAG
CCAGATCTGTGTCAATTACAAGGGCTATTTTAAGTGTGAGTGCTACCCTGGCTACGAGATGGACCTAC
TGACCAAGAACTGCCAAGCTGCTGCTGGAAAGAGCCCATCCCTAATCTTCACCAACCGGTACGAGGTG
CGGAGGATCGACCTGGTGAAGCGGAACTATTCACGCCTCATCCCCATGCTCAAGAATGTCGTGGCACT
AGATGTGGAAGTTGCCACCAATCGCATCTACTGGTGTGACCTCTCCTACCGTAAGATCTATAGCGCCT
ACATGGACAAGGCCAGTGACCCGAAAGAGCAGGAGGTCCTCATTGACGAGCAGTTGCACTCTCCAGAG
GGCCTGGCAGTGGACTGGGTCCACAAGCACATCTACTGGACTGACTCGGGCAATAAGACCATCTCAGT
GGCCACAGTTGATGGTGGCCGCCGACGCACTCTCTTCAGCCGTAACCTCAGTGAACCCCGGGCCATCG
CTGTTGACCCCCTGCGAGGGTTCATGTATTGGTCTGACTGGGGGGACCAGGCCAAGATTGAGAAATCT
GGGCTCAACGGTGTGGACCGGCAAACACTGGTGTCAGACAATATTGAATGGCCCAACGGAATCACCCT
GGATCTGCTGAGCCAGCGCTTGTACTGGGTAGACTCCAAGCTACACCAACTGTCCAGCATTGACTTCA
GTGGAGGCAACAGAAAGACGCTGATCTCCTCCACTGACTTCCTGAGCCACCCTTTTGGGATAGCTGTG
TTTGAGGACAAGGTGTTCTGGACAGACCTGGAGAACGAGGCCATTTTCAGTGCAAATCGGCTCAATGG
CCTGGAAATCTCCATCCTGGCTGAGAACCTCAACAACCCACATGACATTGTCATCTTCCATGAGCTGA
AGCAGCCAAGAGCTCCAGATGCCTGTGAGCTGAGTGTCCAGCCTAATGGAGGCTGTGAATACCTGTGC
CTTCCTGCTCCTCAGATCTCCAGCCACTCTCCCAAGTACACATGTGCCTGTCCTGACACAATGTGGCT
GGGTCCAGACATGAAGAGGTGCTACCGAGCACCTCAATCTACCTCAACTACGACGTTAGCTTCTACCA
TGACGAGGACAGTACCTGCCACCACAAGAGCCCCCGGGACCACCGTCCACAGATCCACCTACCAGAAC
CACAGCACAGAGACACCAAGCCTGACAGCTGCAGTCCCAAGCTCAGTTAGTGTCCCCAGGGCTCCCAG
CATCAGCCCGTCTACCCTAAGCCCTGCAACCAGCAACCACTCCCAGCACTATGCAAATGAAGACAGTA
AGATGGGCTCAACAGTCACTGCCGCTGTTATCGGGATCATCGTGCCCATAGTGGTGATAGCCCTCCTG
TGCATGAGTGGATACCTGATCTGGAGAAACTGGAAGCGGAAGAACACCAAAAGCATGAATTTTGACAA
CCCAGTCTACAGGAAACAAACAGAAGAAGAAGATGAAGATGAGCTCCATATAGGGAGAACTGCTCAGA
TTGGCCATGTCTATCCTGCAGCAATCAGCAGCTTTGATCGCCCACTGTGGGCAGAGCCCTGTCTTGGG
GAGACCAGAGAACCGGAAGACCCAGCCCCTGCCCTCAAGGAGCTTTTTGTCTTGCCGGGGGAACCAAG
GTCACAGCTGCACCAACTCCCGAAGAACCCTCTTTCCGAGCTGCCTGTCGTCAAATCCAAGCGAGTGG
CATTAAGCCTTGAAGATGATGGACTACCCTGAGGATGGGATCACCCCCTTCGTGCCTCATGGAATTCA
GTCCCATGCACTACACTCTGGATGGTGTATGACTGGATGAATGGGTTTCTATATATGGGTCTGTGTGA
GTGTATGTGTGTGTGTGATTTTTTTTTTAAATTTATGTTGCGGAAAGGTAACCACAAAGTTATGATGA
ACTGCAAACATGGAAAGGATGTGAGAGTTTTTCTATGTATAATGTTTTATACACTTTTTAACTGGTTG
CACTACCCATGAGGAATTCGTGGAATGGCTACTGCTGACTAACATGATGCACATAACCAAATGGGGGC
CAATGGCACAGTACCTTACTCATCATTTAAAAACTATATTTACAGAAGATGTTTGGTTGCTGGGGGGG
CTTTTTTGGGTTTTGGGGCATTTGTTTTTTGTAAATAAGATGATTATGCTTTGTGGCTATCCATCAAC
ATAAGTAAAAAAAAAAAAAAAACACTTCAACTCCCTCCCCCATTTAGATTATTTATTAACATATTTTA
AAAATCAGATGAGTTCTATAAATAATTTAGAGAAGTGAGAGTATTTATTTTTGGCATGTTTGGCCCAC
CACACAGACTCTGTGTGTGTATGTGTGTGTTTATATGTGTATGTGTGTGACAGGAAAATCTGTAGAGA
AGAGGCACATCTATGGCTACTGTTCAAATACATAAAGATAAATTTATTTTCACACAGTCCACAAGGGC
TATATCTTGTAGTTTTCAGAAAAGCCTTTGGAAATCTGGATCAGGAAATAGATACCATGGTTTGTGCA
ATTATGTAGTAAAAAGGCAAATCTTTTCACCTCTGGCTATTCCTGAGACCCCAGGAAGTCAGGAAAAA
GCCTTTCAGCTCACCCATGGCTGCTGTGACTCCTACCAGGGCTTTCTTGGCTTTGGCGAAGGTCAGTG
TACAGACATTCCATGGTACCAGAGTGCTCAGAAAGTCAAGATAGGATATGCCTCACCCTCAGCTACTC
CTTGTTTTAAAGTTCAGCTCTTTGAGTAACTTCTTCAATTTCTTTCAGGACACTTGGGTTGAATTCAG
TAAGTTTCCTCTGAAGCACCCTGAAGGGTGCCATCCTTACAGAGCTAAGTGGAGACGTTTCCAGATCA
GCCCAAGTTTACTATAGAGACTGGCCCAGGCACTGAATGTCTAGGACATGCTGTGGATGAAGATAAAG
ATGGTGGAATAGGTTTTATCACATCTCTTATTTCTCTTTTCCCCTTACTCTCTACCATTTCCTTTATG
TGGGGAAACATTTTAAGGTAATAAATAGGTTACTTACCATCATATGTTCATATAGATGAAACTAATTT
TTGGCTTAAGTCAGAACAACTGGCCCCCAATTGAAGTCATATTTGTGGGGGGAAATGGCATACGCAAT
ATTATATTATATTGGATATTTATGTTCACACAGGAATTTGGTTTACTGCTTTGTAAATAAAAGGGAAA
ACTCCGGGTA

| | | | |
|---|---|---|---|
| NOV28a, CG55256-07 | SEQ ID NO:638 | 905 aa | MW at 100461.4 kD |
| Protein Sequence | | | |

MGLPEPGPLRLLALLLLLLLLLLLRLQHLAAAAADPLLGGQGPAKECEKDQFQCRNERCIPSVWRCDE
DDDCLDHSDEDDCPKKTCADSDFTCDNGHCIHERWKCDGEEECPDGSDESEATCTLGTCRGDEFQCGD
GTCVLAIKHCNQEQDCPDGSDEAGCLQESPCEGPRRFQCKSGKRVDGGKVCDVQRDCRDWSDELLKVW
CGACLRPLAGLSLLPSPSWYLGSRPSSAPCPDTFCSDPLFGFMCRPMASHGAFRPQASGLHLYKVLRA
CPSQRRLNECLHNNGGCSHICTDLKIGFECTCPAGFQLLDQKTCGDIDECKDPDACSQICVNYKGYFK
CECYPGYEMDLLTKNCQAAACKSPSLIFTNRYEVRRIDLVKRNYSRLIPMLKNVVALDVEVATNRIYW
CDLSYRKIYSAYMDKASDPKEQEVLIDEQLHSPEGLAVDWVHKHIYWTDSGNKTISVATVDGGRRRTL
FSRNLSEPPAIAVDPLRGFMYWSDWGDQAKIEKSGLNGVDRQTLVSDNIEWPNGITLDLLSQRLYWVD
SKLHQLSSIDFSGGNRKTLISSTDFLSHPFGIAVFEDKVFWTDLENEAIFSANRLNGLEISILAENLN
NPHDIVIFHELKQPHAPDACELSVQPNGGCEYLCLPAPQISSHSPKYTCACPDTMWLGPDMKRCYRAP
QSTSTTTLASTMTRTVPATTRAPGTTVHRSTYQNHSTETPSLTAAVPSSVSVPEAPSISPSTLSPATS
INHSQHYANEDSKMGSTVTAAVIGIIVPIVVIALLCMSGYLIWRAWKRATKSDAFDNPVYRKTTEEED
EDELHIGRTAQIGHVYPAAISSFDRPLWAEPCLGETREPEDPAPALKELFVLPGEPRSQLHQLPKNPL
SELPVVKSKRVALSLEDDGLP

| | | |
|---|---|---|
| NOV28b, CG55256-10 | SEQ ID NO:639 | 1079 bp |
| DNA Sequence | ORF Start: at 1 | ORF Stop: TGA at 1009 |

GCGGCTGATCCGCTGCTCGGCGGCCAAGGGCCGGCCAAGGATTGCGAAAAGGACCAATTCCAGTGCCG
GAACGAGCGCTGCATCCCCTCTGTGTGGAGATGCGACGAGGACGATGACTGCTTAGACCACAGCGACG
AGGACGACTGCCCCAAGAAGACCTGTGCAGACAGTGACTTCACCTGTGACAACGGCCACTGCATCCAC
GAACGGTGGAAGTGTGACGGCGAGGAGGAGTGTCCTGATGGCTCCGATGAGTCCGAGGCCACTTGCAC
CAAGCAGGTGTGTCCTGCAGAGAAGCTGGGTCCAGACATGAAGAGGTGCTACCGAGCACCTCAATCTA
CCTCAACTACGACGTTAGCTTCTACCATGACGAGGACAGTACCTGCCACCACAAGAGCCCCCGGGACC
ACCGTCCACAGATCCACCTACCAGAACCACAGCACAGAGACACCAAGCCTGACAGCTGCAGTCCCAAG
CTCAGTTAGTGTCCCCAGGGCTCCCAGCATCAGCCCGTCTACCCTAAGCCCTGCAACCAGCAACCACT
CCCAGCACTATGCAAATGAAGACAGTAAGATGGGCTCAACAGTCACTGCCGCTGTTATCGGGATCATC
GTGCCCATAGTGGTGATAGCCCTCCTGTGCATGAGTGGATACCTGATCTGGAGAAACTGGAAGCGGAA
GAACACCAAAAGCATGAATTTTGACAACCCAGTCTACAGGAAAACAACAGAAGAAGAAGACGAAGATG
AGCTCCATATAGGGAGAACTGCTCAGATTGGCCATGTCTATCCTGCAGCAATCAGCAGCTTTGATCGC
CCACTGTGGGCAGAGCCCTGTCTTGGGGAGACCAGAGAACCGGAAGACCCAGCCCCTGCCCTCAAGGA
GCTTTTTGTCTTGCCGGGGGAACCAAGGTCACAGCTGCACCAACTCCCGAAGAACCCTCTTTCCGAGC
TGCCTGTCGTCAAATCCAAGCGAGTGGCATTAAGCCTTGAAGATGATGGACTACCCTGAGGATGGGAT
CACCCCCTTCGTGCCTCATGGAATTCAGTCCCATGCACTACACTCTGGATGGTGTATGA

| | | | |
|---|---|---|---|
| NOV28b, CG55256-10 | SEQ ID NO:640 | 336 aa | MW at 36894.7 kD |
| Protein Sequence | | | |

AADPLLGGQGPAKDCEKDQFQCRNERCIPSVWRCDEDDDCLDHSDEDDCPKKTCADSDFTCDNGHCIH
ERWKCDGEEECPDGSDESEATCTKQVCPAEKLGPDMKRCYRAPQSTSTTTLASTMTRTVPATTRAPGT
VPIVVIALLCMSGYLIWRNWKRKNTKSMNFDNPVYRKTTEEEDEDELHIGRTAQIGHVYPAAISSFDR
PLWAEPCLGETREPEDPAPALKELFVLPGEPRSQLHQLPKNPLSELPVVKSKRVALSLEDDGLP

| | | |
|---|---|---|
| NOV28c, 272511714 | SEQ ID NO:641 | 1983 bp |
| DNA Sequence | ORF Start: at 1 | ORF Stop: end of sequence |

GGATCCGCGGCTGATCCGCTGCTCGGCGGCCAAGGGCCGGCCAAGGATTGCGAAAAGGACCAATTCCA
GTGCCGGAACGAGCGCTGCATCCCCTCTGTGTGGAGATGCGACGAGGACGATGACTGCTTAGACCACA
GCGACGAGGACGACTGCCCCAAGAAGACCTGTGCAGACAGTGACTTCACCTGTGACAACGGCCACTGC
ATCCACGAACGGTGGAAGTGTGACGGCGAGGAGGAGTGTCCTGATGGCTCCGATGAGTCCGAGGCCAC
TTGCACCAAGCAGGTGTGTCCTGCAGAGAAGCTGAGCTGTGGACCCACCAGCCACAAGTGTGTACCTG
CCTCGTGGCGCTGCGACGGGGAGAAGGACTGCGAGGGTGGAGCGGATGAGGCCGGCTGTGCTACCTCA
CTGGGCACCTGCCGTGGGGACGAGTTCCAGTGTGGGGATGGGACATGTGTCCTTGCAATCAAGCACTG
CAACCAGGAGCAGGACTGTCCAGATGGGAGTGATGAAGCTGGCTGCCTACAGGGGCTGAACGAGTGTC
GCAGGCTTCCAGCTCCTGGACCAGAAGACCTGTGGCGACATTGATGAGTGCAAGGACCCAGATGCCTG
CAGCCAGATCTGTGTCAATTACAAGGGCTATTTTAAGTGTGAGTGCTACCCTGGCTACGAGATGGACC
TACTGACCAAGAACTGCAAGGCTGCTGCTGGCAAGAGCCCATCCCTAATCTTCACCAACCGGCACGAG
GTGCGGAGGATCGACCTGATGAAGCGGAACTATTCACGCCTCATCCCCATGCTCAAGAATGTCGTGGC
ACTAGATGTGGAAGTTGCCACCAATCGCATCTACTGGTGTGACCTCTCCTACCGTAAGATCTATAGCG
CCTACATGGACAAGGCCAGTGACCCGAAAGAGCAGGAGGTCCTCATTGACGAGCAGTTGCACTCTCCA
GAGGGCCTGGCAGTGGACTGGGTCCACAAGCACATCTACTGGACTGACTCGGGCAATAAGACCATCTC
AGTGGCCACAGTTGATGGTGGCCGCCGACGCACTCTCTTCAGCCGTAACCTCAGTGAACCCCGGGCCA
TCGCTGTTGACCCCCTGCGAGGGTTCATGTATTGGTCTGACTGGGGGGACCAGGCCAAGATTGAGAAA
TCTGGGCTCAACGGTGTGGACCGGCAAACACTGGTGTCAGACAATATTGAATGGCCCAACGGAATCAC
CCTGGATCTGCTGAGCCAGCGCTTGTACTGGGTAGACTCCAAGCTACACCAACTGTCCAGCATTGACT
TCAGTGGAGGCAACAGAAAGACGCTGATCTCCTCCACTGACTTCCTGAGCCACCCTTTTGGGATAGCT
GTGTTTGAGGACAAGGTGTTCTGGACAGACCTGGAGAACGAGGCCATTTTCAGTGCAAATCGGCTCAA
TGGCCTGGAAATCTCCATCCTGGCTGAGAACCTCAACAACCCACATGACATTGTCATCTTCCATGAGC
TGAAGCAGCCAAGAGCTCCAGATGCCTGTGAGCTGAGTGTCCAGCCTAATGGAGGCTGTGAATACCTG
TGCCTTCCTGCTCCTCAGATCTCCAGCCACTCTCCCAAGTACACATGTGCCTGTCCTGACACAATGTG
CCATGACGAGGACAGTACCTGCCACCACAAGAGCCCCCGGGACCACCGTCCACAGATCCACCTACCAG
AACCACAGCACAGAGACACCAAGCCTGACAGCTGCAGTCCCAAGCTCAGTTAGTGTCCCCAGGGTTCC
CAGCATCAGCCCGTCTACCCTAAGACCTGCAACCAGCAACCACTCCCAGCACTATGCAAATGAAGACA
GTAAGCTCGAG

| | | | |
|---|---|---|---|
| NOV28c, 272511714 | SEQ ID NO:642 | 661 aa | MW at 73025.5 kD |
| Protein Sequence | | | |

GSAADPLLGGQGPAKDCEKDQFQCRNERCIPSVWRCDEDDDCLDHSDEDDCPKKTCADSDFTCDNGHC
IHERWKCDGEEECPDGSDESEATCTKQVCPAEKLSCGPTSHKCVPASWRCDGEKDCEGGADEAGCATS
LGTCRGDEFQCGDGTCVLAIKHCNQEQDCPDGSDEAGCLQGLNECLHNNGGCSHICADLKIGFECTCP
AGFQLLDQKTCGDIDECKDPDACSQICVNYKGYFKCECYPGYEMDLLTKNCKAAAGKSPSLIFTNRHE
VRRIDLMKHNYSRLIPMLKNVVALDVEVATNRIYWCDLSYRKIYSAYMDKASDPKEQEVLIDEQLHSP
EGLAVDWVHKHIYWTDSGNKTISVATVDGGRRRTLFSRNLSEPRAIAVDPLRGFMYWSDWGDQAKIEK
SGLNGVDRQTLVSDNIEWPNGITLDLLSQPLYWVDSKLHQLSSIDFSGGNRKTLISSTDFLSHPFGIA
VFEDKVFWTDLENEAIFSANRLNGLEISILAENLNNPHDIVIFHELKQPRAPDACELSVQPNGGCEYL
CLPAPQISSHSPKYTCACPDTMWLGPDMKRCYRAPQSTSTTTLASTMTRTVPATTRAPGTTVHRSTYQ
NHSTETPSLTAAVPSSVSVPRVPSISPSTLRPATSNHSQHYANEDSKLE

| | | |
|---|---|---|
| NOV28d, CG55256-01 | SEQ ID NO:643 | 2537 bp |
| DNA Sequence | ORF Start: ATG at 30 | ORF Stop: TGA at 2505 |

TGTTTTTTTTCTTCAGTACTTTAAATGTCATGCCACTCTCCTGGCCTGTAAGCTTTCCACAGAGAAGC
CTGCTGCCAGACCCATTGGAGCTTCTTTGTGTGTTATTTGTTTCTTTTTTCTTGCTGCTTTTACAGCT
CCAGCATCTTGCGGCGGCAGCGGCTGATCCGCTGCTCGGCGGCCAAGGGCCGGCCAAGGATTGCGAAA
AGGACCAATTCCAGTGCCGGAACGAGCGCTGCATCCCCTCTGTGTGGAGATGCGACGAGGACGATGAC
TGCTTAGACCACAGCGACGAGGACGACTGCCCTGCAGCCAAGAAGACACCTGTGAGAAGTGACTTCAC
CTGTGACAACGGCCACTGCATCCACGAACGGTGGAAGTGTGACGGCGAGGAGGAGTGTCCTGATGGCT
CCGATGAGTCCGAGGCCACTTGCACCAAGCAGGTGTGTCCTGCAGAGAAGCTGAGCTGTGGACCCACT
AGCCACAAGTGTGTACCTGCCTCGTGGCGCTGCGACGGGGAGAAGGACTGCGAGGGTGGAGCGGATGA
GGCCGGCTGTGCTACCTCACTGGGCACCTGCCGTGGGGACGAGTTCCAGTGTGGGGATGGGACATGTG
TCCTTGCAATCAAGCACTGCAACCAGGAGCAGGACTGTCCAGATGGGAGTGATGAAGCTGGCTGCCTA
CAGGGGCTGAACGAGTGTCTGCACAACAATGGCGGCTGCTCACACATCTGCACTGACCTCAAGATTGG
CTTTGAATGCACGTGCCCAGCAGGCTTCCAGCTCCTGGACCAGAAGACCTGTGGCGCTGGCAAGAGCC
CATCCCTAATCTTCACCAACCGGCACGAGGTGCGGAGGATCGACCTGGTGAAGCGGAACTATTCACGC
CTCATCCCCATGCTCAAGAATGTCGTGGCACTAGATGTGGAAGTTGCCACCAATCGCATCTACTGGTG
TGACCTCTCCTACCGTAAGATCTATAGCGCCTACATGGACAAGGCCAGTGACCCGAAAGAGCAGGAGG
TCCTCATTGACGAGCAGTTGCACTCTCCAGAGGGCCTGGCAGTGGACTGGGTCCACAAGCACATCTAC
TGGACTGACTCGGGCAATAAGACCATCTCAGTGGCCACAGTTGATGGTGGCCGCCGACGCACTCTCTT
CAGCCGTAACCTCAGTGAACCCCGGGCCATCGCTGTTGACCCCCTGCGAGGGTTCATGTATTGGTCTG
ACTGGGGGGACCAGGCCAAGATTGAGAAATCTGGGCTCAACGGTGTGGACCGGCAAACACTGGTGTCA
GACAATATTGAATGGCCCAACGGAATCACCTTGGATCTGCTGAGCCAGCGCTTGTACTGGGTAGACTC
CAAGCTACACCAACTGTCCAGCATTGACTTCAGTGGAGGCAACAGAAAGACGCTGATCTCCTCCACTG
ACTTCCTGAGCCACCCTTTTGGGATAGCTGTGTTTGAGGACAAGGTGTTCTGGACAGACCTGGAGAAC
GAGGCCATTTTCAGTGCAAATCGGCTCAATGGCCTGGAAATCTCCATCCTGGCTGAGAACCTGAACAA
CCCACATGACATTGTCATCTTCCATGAGCTGAAGCAGCCAAGAGCTCCAGATGCCTGTGAGCTGAGTG
TCCAGCCTAATGGAGGCTGTGAATACCTGTGCCTTCCTGCTCCTCAGATCTCCAGCCACTCTCCCAAG
TACACATGTGCCTGTCCTGACACAATGTGGCTGGGTCCAGACATGAAGAGGTGCTACCGAGCACCTCA
ATCTACCTCAACTACGACGTTAGCTTCTACCATGACGAGGACAGTACCTGCCACCACAAGAGCCCCCG
GGACCACCGTCCACAGATCCACCTACCAGAACCACAGCACAGAGACACCAAGCCTGACAGCTGCAGTC
CCAAGCTCAGTTAGTGTCCCCAGGGCTCCCAGCATCAGCCCGTCTACCCTAAGCCCTGCAACCAGCAA
CCACTCCCAGCACGCAAATGAAGACAGTAAGATGGGCTCAACAGTCACTGCCGCTGTTATCGGGATCA
TCGTGCCCATAGCAAATGAAGACAGTAAGATGGGCTCAACAGTCACTGCCGCTGTTATCGGGATCATC
GTGCCCATAGGTGATAGCCCTCCTGTGCATGAGTGGATACCTGATCTGGAGAAACTGGAAGCGGAAGA
ACACCAAAAGCATGAATTTGACAACCCAGTCTACAGGAAAACAACAGAAGAAGAAGACGAAGATGAGC
TCCATATAGGGAGAACTGCTCAGATTGGCCATGTCTATCCTGCAGCAATCAGCAGCTTTGATCGCCCA
CTGTGGGCAGAGCCCTGTCTTGGGGAGACCAGAGAACCAGAAGACCCAGCCCCTGCCCTGAAGGAGCT
TTTTGTCTTGCCGGGGGAACCAAGGTCACAGCTGCACCAACTCCCGAAGAACCCTCTTTCCGAGCTGC
CTGTCGTCAAATCCAAGCAGCGAGTGGCATTAAGCCTTGAAGATGATGGACTACCCTGAGGATGGGAT
CACCCCCTTCGTGCCTCATGG

| | | | |
|---|---|---|---|
| NOV28d, CG55256-01 | SEQ ID NO:644 | 1825 aa | MW at 90830.8 kD |
| Protein Sequence | | | |

MPLSWPVSFPQRSLLPDPLELLCVLFVSFFLLLLQLQHLAAAAADPLLGGQGPAKDCEKDQFQCRNER
CIPSVWRCDEDDDCLDHSDEDDCPAAKKTPVRSDFTCDNGHCIHERWKCDGEEECPDGSDESEATCTK
QVCPAEKLSCGPTSHKCVPASWRCDGEKDCEGGADAGCATSLGTCRGDEFQCGDGTCVIAAIAACNQE
QDCPDGSDEAGCLQGLNECLHNNGGCSHICTDLKIGFECTCPAGFQLLDQKTCGAGKSPSLIFTNRHE
VRRIDLVKRNYSRLIPMLKNVVALDVEVATNRIYWCDLSYRKIYSAYMDKASDPKEQEVLIDEQLHSP
EGLAVDWVHKHIYWTDSGNKTISVATVDGGRPRTLFSRNLSEPRAIAVDPLRGFMYWSDWGDQAKIEK
SGLNGVDRQTLVSDNIEWPNGITLDLLSQRLYWVDSKLHQLSSIDFSGGNRKTLISSTDFLSHPFGIA
VFEDKVFWTDLENEAIFSANRLNGLEISILAENLNNPHDIVIFHELKQPRAPDACELSVQPNGGCEYL
CLPAPQISSHSPKYTCACPDTMWLGPDMKRCYRAPQSTSTTTLASTMTRTVPATTRAPGTTVHRSTYQ
NHSTETPSLTAAVPSSVSVPRAPSISPSTLSPATSNHSQHANEDSKMGSTVTAAVIGIIVPIANEDSK
MGSTVTAAVIGIIVPIGDSPPVHEWIPDLEKLEAEEHQKHEFDNPVYRKTTEEEDEDELHIGRTAQIG
HVYPAAISSFDRPLWAEPCLGETREPEDPAPALKELFVLPGEPRSQLHQLPKNPLSELPVVKSKQRVA
LSLEDDGLP

| | | |
|---|---|---|
| NOV28e, CG55256-02 | SEQ ID NO:645 | 3039 bp |
| DNA Sequence | ORF Start: ATG at 1 | ORF Stop: TGA at 3037 |

ATGGGCCTCCCCGAGCCGGGCCCTCTCCGGCTTCTGGCGCTGCTGCTGCTGCTGCTGCTGCTGCTGCT
AGTGCGAAAAGGACCAATTCCAGTGCCGGAACGAGCGCTGCATCCCCTCTGTGTGGAGATGCGACGAG
GACGATGACTGCTTAGACCACAGCGACGAGGACGACTGCCCCAAGAAGACCTGTGCAGACAGTGACTT
CACCTGTGACAACGGCCACTGCATCCACGAACGGTGGAAGTGTGACGGCGAGGAGGAGTGTCCTGATG
GCTCCGATGAGTCCGAGGCCACTTGCACCAAGCAGGTGTGTCCTGCAGAGAAGCTGAGCTGTGGACCC
ACCAGCCACAAGTGTGTACCTGCCTCGTGGCGCTGCGACGGGGAGAAGGACTGCGAGGGTGGAGCGGA
TGAGGCCGGCTGTGCTACCTTGTGCGCCCCGCACGAGTTCCAGTGCGGCAACCGCTCGTGCCTGGCCG
CCGTGTTCGTGTGCGACGGCGATGACGACTGTGGTGACGGCAGCGATGAGCGCGGCTGTGCAGACCCG
GCCTGCGGGCCCCGCGAGTTCCGCTGCGGCGGCGATGGCGGCGGCGCCTGCATCCCGGAGCGCTGGGT
CTGCGACCGCCAGTTTGACTGCGAGGACCGCTCGGACGAGGCAGCCGAGCTCTGCGGCCGCCCGGGCC
CCGGGGCCACGTCCGCGCCCGCCGCCTGCGCCACCGTCTCCCAGTTCGCCTGCCGCAGCGGCGAGTGC
ACTGGGCACCTGCCGTGGGGACGAGTTCCAGTGTGGGGATGGGACATGTGTCCTTGCAATCAAGCACT
GGTCCCCGCAGATTTCAGTGTAAGAGTGGCAAGCGCGTGGACGGCGGGAAAGTGTGTGATGTGCAGAG
GGACTGCCGGGACTGGTCGGATGAGCTTCTGAAAGTGTGGTGCGTCTCCTACCATCCCCCTCCTGGGC
TGAACGAGTGTCTGCACAACAATGGCGGCTGCTCACACATCTGCACTGACCTCAAGATTGGCTTTGAA
TGCACGTGCCCAGCAGGCTTCCAGCTCCTGGACCAGAAGACCTGTGGCGACATTGATGAGTGCAAGGA
CCCAGATGCCTGCAGCCAGATCTGTGTCAATTACAAGGGCTATTTTAAGTGTGAGTGCTACCCTGGCT
ACGAGATGGACCTACTGACCAAGAACTGCAAGGCTGCTGCTGGAAAGAGCCCATCCCTAATCTTCACC
AACCGGCACGAGGTGCGGAGGATCGACCTGGTGAAGCGGAACTATTCACGCCTCATCCCCATGCTCAA
GAATGTCGTGGCACTAGATGTGGAAGTTGCCACCAATCGCATCTACTGGTGTGACCTCTCCTACCGTA
AGATCTATAGCGCCTACATGGACAAGGCCAGTGACCCGAAAGAGCAGGAGGTCCTCATTGACGAGCAG
TTGCACTCTCCAGAGGGCCTGGCAGTGGACTGGGTCCACAAGCACATCTACTGGACTGACTCGGGCAA
TAAGACCATCTCAGTGGCCACAGTTGATGGTGGCCGCCGACGCACTCTCTTCAGCCGTAACCTCAGTG
AACCCCGGGCCATCGCTGTTGACCCCCTGCGAGGGTTCATGTATTGGTCTGACTGGGGGGACCAGGCC
AAGATTGAGAAATCTGGGCTCAACGGTGTGGACCGGCAAACACTGGTGTCAGACAATATTGAATGGCC
CAACGGAATCACCCTGGATCTGCTGAGCCAGCGCTTGTACTGGGTAGACTCCAAGCTACACCAACTGT
CCAGCATTGACTTCAGTGGAGGCAACAGAAAGACGCTGATCTCCTCCACTGACTTCCTGAGCCACCCT
TTTGGGATAGCTGTGTTTGAGGACAAGGTGTTCTGGACAGACCTGGAGAACGAGGCCATTTTCAGTGC
AAATCGGCTCAATGGCCTGGAAATCTCCATCCTGGCTGAGAACCTCAACAACCCACATGACATTGTCA
TCTTCCATGAGCTGAAGCAGCCAAGAGCTCCAGATGCCTGTGAGCTGAGTGTCCAGCCTAATGGAGGC
TGTGAATACCTGTGCCTTCCTGCTCCTCAGATCTCCAGCCACTCTCCCAAGTACACATGTGCCTGTCC
TGACACAATGTGGCTGGGTCCAGACATGAAGAGGTGCTACCGAGCACCTCAATCTACCTCAACTACGA
CGTTAGCTTCTACCATGACGAGGACAGTACCTGCCACCACAAGAGCCCCCGGGACCACCGTCCACAGA
TCCACCTACCAGAACCACAGCACAGAGACACCAAGCCTGACAGCTGCAGTCCCAAGCTCAGTTAGTGT
CAAATGAAGACAGTAAGATGGGCTCAACAGTCACTGCCGCTGTTATCGGGATCATCGTGCCCATAGTG
GTGATAGCCCTCCTGTGCATGAGTGGATACCTGATCTGGAGAAACTGGAAGCGGAAGAACACCAAAAG
CATGAATTTTGACAACCCAGTCTACAGGAAAACAACAGAAGAAGAAGATGAAGATGAGCTCCATATAG
GAGCCCTGTCTTGGGGAGACCAGAGAACCGGAAGACCCAGCCCCTGCCCTCAAGGAGCTTTTTGTCTT
GCCGGGGGAACCAAGGTCACAGCTGCACCAACTCCCGAAGAACCCTCTTTCCGAGCTGCCTGTCGTCA
AATCCAAGCGAGTGGCATTAAGCCTTGAAGATGATGGACTACCCTGA

| | | | |
|---|---|---|---|
| NOV28e, CG55256-02 | SEQ ID NO:646 | 1012 aa | MW at 111334.6 kD |
| Protein Sequence | | | |

MGLPEPGPLRLLALLLLLLLLLLLRLQHLAAAAADPLLGGQGPAKECEKDQFQCRNERCIPSVWRCDE
DDDCLDHSDEDDCPKKTCADSDFTCDNGHCIHERWKCDGEEECPDGSDESEATCTKQVCPAEKLSCGP
TSHKCVPASWRCDGEKDCEGGADEAGCATLCAPHEFQCGNRSCLAAVFVCDGDDDCGDGSDERGCADP
ACGPREFRCGGDGGGACIPERWVCDRQFDCEDRSDEAAELCGRPGPGATSAPAACATVSQFACRSGEC
VHLGWRCDGDRDCKDKSDEADCPLGTCRGDEFQCGDGTCVLAIKHCNQEQDCPDGSDEAGCLQESPCE
GPRRFQCKSGKRVDGGKVCDVQRDCRDWSDELLKVWCVSYHPPPGLNECLHNNGGCSHICTDLKIGFE
CTCPAGFQLLDQKTCGDIDECKDPDACSQICVNYKGYFKCECYPGYEMDLLTKNCKAAAGKSPSLIFT
NRHEVRRIDLVKRNYSRLIPMLKNVVALDVEVATNRIYWCDLSYRKIYSAYMDKASDPKEQEVLIDEQ
LHSPEGLAVDWVHKHIYWTDSGNKTISVATVDGGRRRTLFSRNLSEPAAIAVDPLRGFMYWSDWGDQA
KIEKSGLNGVDRQTLVSDNIEWPNGITLDLLSQRLYWVDSKLHQLSSIDFSGGNRKTLISSTDFLSHP
FGIAVFEDKVFWTDLENEAIFSANRLNGLEISILAENLNNPHDIVIFHELKQPRAPDACELSVQPNGG
CEYLCLPAPQISSHSPKYTCACPDTMWLGPDMKRCYRAPQSTSTTTLASTMTRTVPATTRAPGTTVHR
STYQNHSTETPSLTAAVPSSVSVPRAPSISPSTLSPATSNHSQHYANEDSKMGSTVTAAVIGIIVPIV
VIALLCMSGYLIWRNWKRKNTKSMNFDNPVYRKTTEEEDEDELHIGRTAQIGHVYPAAISSFDRPLWA
EPCLGETREPEDPAPALKELFVLPGEPRSQLHQLPKNPLSELPVVKSKRVALSLEDDGLP

| | | |
|---|---|---|
| NOV28f, CG55256-03 | SEQ ID NO:647 | 2537 bp |
| DNA Sequence | ORF Start: ATG at 1 | ORF Stop: TGA at 2086 |

ATGGGCCTCCCCGAGCCGGGCCCTCTCCGGCTTCTGGCGCTGCTGCTGCTGCTGCTGCTGCTGCTGCT
GCTGCGGCTCCAGCATCTTGCGGCGGCAGCGGCTGATCCGCTGCTCGGCGGCCAAGGGCCGGCCAAGG
ATTGCGAAAAGGACCAATTCCAGTGCCGGAACGAGCGCTGCATCCCCTCTGTGTGGAGATGCGACGAG
GACGATGACTGCTTAGACCACAGCGACGAGGACGACTGCCCCAAGAAGACCTGTGCAGACAGTGACTT
CACCTGTGACAACGGCCACTGCATCCACGAACGGTGGAAGTGTGACGGCGAGGAGGAGTGTCCTGATG
GCTCCGATGAGTCCGAGGCCACTTGCACCAAGCAGGTGTGTCCTGCAGAGAAGCTGAGCTGTGGACCC
ACCAGCCACAAGTGTGTACCTGCCTCGTGGCGCTGCGACGGGGAGAAGGACTGCGAGGGTGGAGCGGA
TGAGGCCGGCTGTGCTACCTCACTGGGCACCTGCCGTGGGGACGAGTTCCAGTGTGGGGATGGGACAT
GTGTCCTTGCAATCAAGCACTGCAACCAGGAGCAGGACTGTCCAGATGGGAGTGATGAAGCTGGCTGC
CTACAGGGGCTGAACGAGTGTCTGCACAACAATGGCGGCTGCTCACACATCTGCACTGACCTCAAGAT
AGTGCAAGGACCCAGATGOCTGCAGCCAGATCTGTGTCAATTACAAGGGCTATTTTAAGTGTGAGTGC
TACCCTGGCTACGAGATGGACCTACTGACCAAGAACTGCAAGGCTGCTGCTGGAAAGAGCCCATCCCT
AATCTTCACCAACCGGCACGAGGTGCGGAGGATCGACCTGGTGAAGCGGAACTATTCACGCCTCATCC
CCATGCTCAAGAATGTCGTGGCACTAGATGTGGAAGTTGCCACCAATCGCATCTACTGGTGTGACCTC
TCCTACCGTAAGATCTATAGCGCCTACATGGACAAGGCCAGTGACCCGAAAGAGCAGGAGGTCCTCAT
TGACGAGCAGTTGCACTCTCCAGAGGGCCTGGCAGTGGACTGGGTCCACAAGCACATCTACTGGACTG
ACTCGGGCAATAAGAACATCTCTGGTGGCCGCCGACGCACTCTCTTCAGCCGTAACCTCAGTGAACCC
CGGGCCATCGCTGTTGACCCCCTGCGAGGGTTCATGTATTGGTCTGACTGGGGGGACCAGGCCAAGAT
TGAGAAATCTGGGCTCAACGGTGTGGACCGGCAAACACTGGTGTCAGACAATATTGAATGGCCCAACG
GAATCACCCTGGATCTGCTGAGCCAGCGCTTGTACTGGGTAGACTCCAAGCTACACCAACTGTCCAGC
ATTGACTTCAGTGGAGGCAACAGAAAGACGCTGATCTCCTCCACTGACTTCCTGAGCCACCCTTTTGG
GATAGCTGTGTTTGAGGACAAGGTGTTCTGGACAGACCTGGAGAACGAGGCCATTTTCAGTGCAAATC
GGCTCAATGGCCTGGAAATCTCCATCCTGGCTGAGAACCTCAACAACCCACATGACATTGTCATCTTC
CATGAGCTGAAGCAGCCAAGAGCTCCAGATGCCTGTGAGCTGAGTGTCCAGCCTAATGGAGGCTGTGA
ATACCTGTGCCTTCCTGCTCCTCAGATCTCCAGCCACTCTCCCAAGTACACATGTGCCTGTCCTGACA
CAATGTGGCTGGGTCCAGACATGAAGAGGTGCTACCGAGATGCAAATGAAGACAGTAAGATGGGCTCA
ACAGTCACTGCCGCTGTTATCGGGATCATCGTGCCCATAGTGGTGATAGCCCTCCTGTGCATGAGTGG
ATACCTGATCTGGAGAAACTGGAAGCGGAAGAACACCAAAAGCATGAATTTTGACAACCCAGTCTACA
GGAAAACAACAGAAGAAGAAGATGAAGATGAGCTCCATATAGGGAGAACTGCTCAGATTGGCCATGTC
TATCCTGCACGAGTGGCATTAAGCCTTGAAGATGATGGACTACCCTGAGGATGGGATCACCCCCTTCG
TGCCTCATGGAATTCAGTCCCATGCACTACACTCCGGATGGTGTATGACTGGATGAATGGGTTTCTAT
ATATGGGTCTGTGTGAGTGTATGTGTGTGTGTGATTTTTTTTTTTTAAATTATGTTGCGGAAAGGTAA
CCACAAAGTTATGATGAACTGCAAACATCCAAAGGATGTGAGAGTTTTTCTATGTATAATGTTTTATA
CACTTTTTAACTGGTTGCACTACCCATGAGGAATTCGTGGAATGGCTACTGCTGACTAACATGATGCA
CATAACCAAATGGGGGCCAATGGCACAGTACCTTACTCATCATTTAAAAACTATATTTACAGAAGATG
TTTGGTTGCTGGGGGGCTTTTTTAGGTTTTGGGCATTTGTTTTTTGTAAATAAGATGATTATGCTTTG
TGGCTATCCATCAACATAAGT

| | | | |
|---|---|---|---|
| NOV28f, CG55256-03 | SEQ ID NO:648 | 695 aa | MW at 77399.4 kD |
| Protein Sequence | | | |

MGLPEPGPLRLLALLLLLLLLLLLRLQHLAAAAADPLLGGQGPAKDCEKDQFQCRNERCIPSVWRCDE
DDDCLDHSDEDDCPKKTCADSDFTCDNGHCIHERWKCDGEEECPDGSDESEATCTKQVCPAEKLSCGP
TSHKCVPASWRCDGEKDCEGGADEAGCATSLGTCRGDEFQCGDGTCVLAIKHCNQEQDCPDGSDEAGC
LQGLNECLHNNGGCSHICTDLKIGFECTCPAGFQLLDQKTCGDIDECKDPDACSQICVNYKGYFKCEC
YPGYEMDLLTKNCKAAAGKSPSLIFTNRHEVRRIDLVKRNYSRLIPMLKNVVALDVEVATNRIYWCDL
SYRKIYSAYMDKASDPKEQEVLIDEQLHSPEGLAVDWVHKHIYWTDSGNKNISGGRRRTLFSRNLSEP
RAIAVDPLRGFMYWSDWGDQAKIEKSGLNGVDRQTLVSDNIEWPNGITLDLLSQRLYWVDSKLHQLSS
IDFSGGNRKTLISSTDFLSHPFGIAVFEDKVFWTDLENEAIFSANRLNGLEISILAENLNNPHDIVIF
HELKQPRAPDACELSVQPNGGCEYLCLPAPQISSHSPKYTCACPDTMWLGPDMKRCYRDANEDSKMGS
TVTAAVIGIIVPIVVIALLCMSGYLIWRNWKRKNTKSMNFDNPVYRKTTEEEDEDELHIGRTAQIGHV
YPARVALSLEDDGLP

| | | |
|---|---|---|
| NOV28g, CG55256-04 | SEQ ID NO:649 | 2474 bp |
| DNA Sequence | ORF Start: ATG at 30 | ORF Stop: TGA at 2442 |

TGTTTTTTTTCTTCAGTACTTTAAATGTCATGCCACTCTCCTGGCCTGTAAGCTTTCCACAGAGAAGC
CTGCTGCCAGACCCATTGGAGCTTCTTTGTGTGTTATTTGTTTCTTTTTTCTTGCTGCTTTTACAGCT
CCAGCATCTTGCGGCGGCAGCGGCTGATCCGCTGCTCGGCGGCCAAGGGCCGGCCAAGGATTGCGAAA
AGGACCAATTCCAGTGCCGGAACGAGCGCTGCATCCCCTCTGTGTGGAGATGCGACGAGGACGATGAC
TGCTTAGACCACAGCGACGAGGACGACTGCCCTGCAGCCAAGAAGACACCTGTGAGAAGTGACTTCAC
CTGTGACAACGGCCACTGCATCCACGAACGGTGGAAGTGTGACGGCGAGGAGGAGTGTCCTGATGGCT
CCGATGAGTCCGAGGCCACTTGCACCAAGCAGGTGTGTCCTGCAGAGAAGCTGAGCTGTGGACCCACT
AGCCACAAGTGTGTACCTGCCTCGTGGCGCTGCGACGGGGAGAAGGACTGCGAGGGTGGAGCGGATGA
TCCTTGCAATCAAGCACTGCAACCAGGAGCAGGACTGTCCAGATGGGAGTGATGAAGCTGGCTGCCTA
CTTTGAATGCACGTGCCCAGCAGGCTTCCAGCTCCTGGACCAGAAGACCTGTGGCGCTGGCAAGAGCC
CATCCCTAATCTTCACCAACCGGCACGAGGTGCGGAGGATCGACCTGGTGAAGCGGAACTATTCACGC
CTCATCCCCATGCTCAAGAATGTCGTGGCACTAGATGTGGAAGTTGCCACCAATCGCATCTACTGGTG
TGACCTCTCCTACCGTAAGATCTATAGCGCCTACATGGACAAGGCCAGTGACCCGAAAGAGCAGGAGG
TCCTCATTGACGAGCAGTTGCACTCTCCAGAGGGCCTGGCAGTGGACTGGGTCCACAAGCACATCTAC
TGGACTGACTCGGGCAATAAGACCATCTCAGTGGCCACAGTTGATGGTGGCCGCCGACGCACTCTCTT
CAGCCGTAACCTCAGTGAACCCCGGGCCATCGCTGTTGACCCCCTGCGAGGGTTCATGTATTGGTCTG
ACTGGGGGGACCAGGCCAAGATTGAGAAATCTGGGCTCAACGGTGTGGACCGGCAAACACTGGTGTCA
GACAATATTGAATGGCCCAACGGAATCACCTTGGATCTGCTGAGCCAGCGCTTGTACTGGGTAGACTC
CAAGCTACACCAACTGTCCAGCATTGACTTCAGTGGAGGCAACAGAAAGACGCTGATCTCCTCCACTG
ACTTCCTGAGCCACCCTTTTGGGATAGCTGTGTTTGAGGACAAGGTGTTCTGGACAGACCTGGAGAAC
GAGGCCATTTTCAGTGCAAATCGGCTCAATGGCCTGGAAATCTCCATCCTGGCTGAGAACCTGAACAA
CCCACATGACATTGTCATCTTCCATGAGCTGAAGCAGCCAAGAGCTCCAGATGCCTGTGAGCTGAGTG
TCCAGCCTAATGGAGGCTGTGAATACCTGTGCCTTCCTGCTCCTCAGATCTCCAGCCACTCTCCCAAG
TACACATGTGCCTGTCCTGACACAATGTGGCTGGGTCCAGACATGAAGAGGTGCTACCGAGCACCTCA
ATCTACCTCAACTACGACGTTAGCTTCTACCATGACGAGGACAGTACCTGCCACCACAAGAGCCCCCG
GGACCACCGTCCACAGATCCACCTACCAGAACCACAGCACAGAGACACCAAGCCTGACAGCTGCAGTC
CCAAGCTCAGTTAGTGTCCCCAGGGCTCCCAGCATCAGCCCGTCTACCCTAAGCCCTGCAACCAGCAA
CCACTCCCAGCACTATGCAAATGAAGACAGTAAGATGGGCTCAACAGTCACTGCCGCTGTTATCGGGA
TCATCGTGCCCATAGTGGTGATAGCCCTCCTGTGCATGAGTGGATACCTGATCTGGAGAAACTGGAAG
CGGAAGAACACCAAAAGCATGAATTTTGACAACCCAGTCTACAGGAAAACAACAGAAGAAGAAGACGA
AGATGAGCTCCATATAGGGAGAACTGCTCAGATTGGCCATGTCTATCCTGCAGCAATCAGCAGCTTTG
ATCGCCCACTGTGGGCAGAGCCCTGTCTTGGGGAGACCAGAGAACCGGAAGACCCAGCCCCTGCCCTC
AAGGAGCTTTTTGTCTTGCCGGGGGAACCAAGGTCACAGCTGCACCAACTCCCGAAGAACCCTCTTTC
CGAGCTGCCTGTCGTCAAATCCAAGCGAGTGGCATTAAGCCTTGAAGATGATGGACTACCCTGAGGAT
GGGATCACCCCCTTCGTGCCTCATGG

| | | | |
|---|---|---|---|
| NOV28g, CG55256-04 | SEQ ID NO:650 | 804 aa | MW at 88873.0 kD |
| Protein Sequence | 1804 | | |

MPLSWPVSFPQRSLLPDPLELLCVLFVSFFLLLLQLQHLAAAAADPLLGGQGPAKDCEKDQFQCRNER
CIPSVWRCDEDDDCLDHSDEDDCPAAKKTPVRSDFTCDNGHCIHERWKCDGEEECPDGSDESEATCTK
QVCPAEKLSCGPTSHKCVPASWRCDGEKDCEGGADEAGCATSLGTCRGDEFQCGDGTCVLAIKHCNQE
QDCPDGSDEAGCLQGLNECLHNNGGCSHICTDLKIGFECTCPAGFQLLDQKTCGAGKSPSLIFTNRHE
VRRIDLVKRNYSRLIPMLKNVVALDVEVATNRIYWCDLSYRKIYSAYMDKASDPKEQEVLIDEQLHSP
EGLAVDWVHKHIYWTDSGNKTISVATVDGGRRRTLFSRNLSEPRAIAVDPLRGFMYWSDWGDQAKIEK
SGLNGVDRQTLVSDNIEWPNGITLDLLSQRLYWVDSKLHQLSSIDFSGGNRKTLISSTDFLSHPFGIA
VFEDKVFWTDLENEAIFSANRLNGLEISILAENLNNPHDIVIFHELKQPRAPDACELSVQPNGGCEYL
CLPAPQISSHSPKYTCACPDTMWLGPDMKRCYRAPQSTSTTTLASTMTRTVPATTAAPGTTVHRSTYQ
NHSTETPSLTAAVPSSVSVPPAPSISPSTLSPATSNHSQHYANEDSKMGSTVTAAVIGIIVPIVVIAL
LCMSGYLIWRNWKRKNTKSMNFDNPVYRKTTEEEDEDELHIGRTAQIGHVYPAAISSFDRPLWAEPCL
GETREPEDPAPALKELFVLPGEPRSQLHQLPKNPLSELPVVKSKRVALSLEDDGLP

| | | |
|---|---|---|
| +TL,1NOV28h, CG55256-05 | SEQ ID NO:651 | 4150 bp |
| DNA Sequence | ORF Start: ATG at 103 | ORF Stop: TGA at 2674 |

GCAGCGGCAACCCCGGCGCCGCGGCAAGGACTCGGAGGGCTGAGACGCGGCGGCGGCGGCGCGGGGAC
CGCGGGGCGCGGCGGCCGGAGCCCCGGGCCCGCCATGGGCCTCCCCGAGCCGGGCCCTCTCCGGCTTC
TGGCGCTGCTGCTGCTGCTGCTGCTGCTGCTGCTGCTGCGGCTCCAGCATCTTGCGGCGGCAGCGGCT
GATCCGCTGCTCGGCGGCCAAGGGCCGGCCAAGGAGTGCGAAAAGGACCAATTCCAGTGCCGGAACGA
GCGCTGCATCCCCTCTGTGTGGAGATGCGACGAGGACGATGACTGCTTAGACCACAGCGACGAGGACG
ACTGCCCCAAGAAGACCTGTGCAGACAGTGACTTCACCTGTGACAACGGCCACTGCATCCACGAACGG
TGGAAGTGTGACGGCGAGGAGGAGTGTCCTGATGGCTCCGATGAGTCCGAGGCCACTTGCACACTGGG
CACCTGCCGTGGGGACGAGTTCCAGTGTGGGGATGGGACATGTGTCCTTGCAATCAAGCACTGCAACC
AGGAGCAGGACTGTCCAGATGGGAGTGATGAAGCTGGCTGCCTACAGGAGTCACCCTGTGAGGGTCCC
CGCAGATTTCAGTGTAAGAGTGGCAAGCGCGTGGACGGCGGGAAAGTGTGTGATGTGCAGAGGGACTG
CCGGGACTGGTCGGATGAGCTTCTGAAAGTGTGGTGCGGTGCCTGTCTACGCCCACTGGCTGGACTCA
GTCTCCTACCATCCCCCTCCTGGAGCCCATGTCCCTCCCCAGGGCTGAACGAGTGTCTGCACAACAAT
GGCGGCTGCTCACACATCTGCACTGACCTCAAGATTGGCTTTGAATGCACGTGCCCAGCAGGCTTCCA
GCTCCTGGACCAGAAGACCTGTGGCGACATTGATGAGTGCAAGGACCCAGATGCCTGCAGCCAGATCT
AACTGCAAGGCTGCTGCTGGCAAGAGCCCATCCCTAATCTTCACCAACCGGCACGAGGTGCGGAGGAT
CGACCTGGTGAAGCGGAACTATTCACGCCTCATCCCCATGCTCAAGAATGTCGTGGCACTAGATGTGG
AAGTTGCCACCAATCGCATCTACTGGTGTGACCTCTCCTACCGTAAGATCTATAGCGCCTACATGGAC
AGTGGACTGGGTCCACAAGCACATCTACTGGACTGACTCGGGCAATAAGACCATCTCAGTGGCCACAG
TTGATGGTGGCCGCCGACGCACTCTCTTCAGCCGTAACCTCAGTGAACCCCGGGCCATCGCTGTTGAC
CCCCTGCGAGGGTTCATGTATTGGTCTGACTGGGGGGACCAGGCCAAGATTGAGAAATCTGGGCTCAA
CGGTGTGGACCGGCAAACACTGGTGTCAGACAATATTGAATGGCCCAACGGAATCACCCTGGATCTGC
TGAGCCAGCGCTTGTACTGGGTAGACTCCAAGCTACACCAACTGTCCAGCATTGACTTCAGTGGAGGC
AACAGAAAGACGCTGATCTCCTCCACTGACTTCCTGAGCCACCCTTTTGGGATAGCTGTGTTTGAGGA
CAAGGTGTTCTGGACAGACCTGGAGAACGAGGCCATTTTCAGTGCAAATCGGCTCAATGGCCTGGAAA
AGAGCTCCAGATGCCTGTGAGCTGAGTGTCCAGCCTAATGGAGGCTGTGAATACCTGTGCCTTCCTGC
TCCTCAGATCTCCAGCCACTCTCCCAAGTACACATGTGCCTGTCCTGACACAATGTGGCTGGGTCCAG
ACATGAAGAGGTGCTACCGAGCACCTCAATCTACCTCAACTACGACGTTAGCTTCTACCATGACGAGG
ACAGTACCTGCCACCACAAGAGCCCCCGGGACCACCGTCCACAGATCCACCTACCAGAACCACAGCAC
AGAGACACCAAGCCTGACAGCTGCAGTCCCAAGCTCAGTTAGTGTCCCCAGGGCTCCCAGCATCAGCC
CGTCTACCCTAAGCCCTGCAACCAGCAACCACTCCCAGCACTATGCAAATGAAGACAGTAAGATGGGC
TCAACAGTCACTGCCGCTGTTATCGGGATCATCGTGCCCATAGTGGTGATAGCCCTCCTGTGCATGAG
TGGATACCTGATCTGGAGAAACTGGAAGCGGAAGAACACCAAAAGCATGAATTTTGACAACCCAGTCT
ACAGGAAAACAACAGAAGAAGAAGATGAAGATGAGCTCCATATAGGGAGAACTGCTCAGATTGGCCAT
GTCTATCCTGCAGCAATCAGCAGCTTTGATCGCCCACTGTGGGCAGAGCCCTGTCTTGGGGAGACCAG
AGAACCGGAAGACCCAGCCCCTGCCCTCAAGGAGCTTTTTGTCTTGCCGGGGGAACCAAGGTCACAGC
TGCACCAACTCCCGAAGAACCCTCTTTCCGAGCTGCCTGTCGTCAAATCCAAGCGAGTGGCATTAAGC
CTTGAAGATGATGGACTACCCTGAGGATGGGATCACCCCCTTCGTGCCTCATGGAATTCAGTCCCATG
CACTACACTCTGGATGGTGTATGACTGGATGAATGGGTTTCTATATATGGGTCTGTGTGAGTGTATGT
GTGTGTGTGATTTTTTTTTTAAATTTATGTTGCGGAAAGGTAACCACAAAGTTATGATGAACTGCAAA
CATCCAAAGGATGTGAGAGTTTTTCTATGTATAATGTTTTATACACTTTTTAACTGGTTGCACTACCC
ATGAGGAATTCGTGGAATGGCTACTGCTGACTAACATGATGCACATAACCAAATGGGGGCCAATGGCA
CAGTACCTTACTCATCATTTAAAAACTATATTTACAGAAGATGTTTGGTTGCTGGGGGGGCTTTTTTG
GGTTTTGGGGCATTTGTTTTTTGTAAATAAGATGATTATGCTTTGTGGCTATCCATCAACATAAGTAA
AAAAAAAAAAAAAACACTTCAACTCCCTCCCCCATTTAGATTATTTATTAACATATTTTAAAAATCAG
ATGAGTTCTATAAATAATTTAGAGAAGTGAGAGTATTTATTTTTGGCATGTTTGGCCCACCACACAGA
CTCTGTGTGTGTATGTGTGTGTTTATATGTGTATGTGTGTGACAGGAAAATCTGTAGAGAAGAGGCAC
ATCTATGGCTACTGTTCAAATACATAAAGATAAATTTATTTTCACACAGTCCACAAGGGGTATATCTT
GTAGTTTTCAGAAAAGCCTTTGGAAATCTGGATCAGGAAATAGATACCATGGTTTGTGCAATTATGTA
GTAAAAAAGGCAAATCTTTTCACCTCTGGCTATTCCTGAGACCCCAGGAAGTCAGGAAAAGCCTTTCA
GCTCACCCATGGCTGCTGTGACTCCTACCAGGGCTTTCTTGGCTTTGGCGAAGGTCAGTGTACAGACA
TTCCATGGTACCAGAGTGCTCAGAAAGTCAAGATAGGATATGCCTCACCCTCAGCTACTCCTTGTTTT
CTCTGAAGCACCCTGAAGGGTGCCATCCTTACAGAGCTAAGTGGAGACGTTTCCAGATCAGCCCAAGT
TTACTATAGAGACTGGCCCAGGCACTGAATGTCTAGGACATGCTGTGGATGAAGATAAAGATGGTGGA
ATAGGTTTTATCACATCTCTTATTTCTCTTTTCCCCTTACTCTCTACCATTTCCTTTATGTGGGGAAA
CATTTTAAGGTAATAAATAGGTTACTTACCATCATATGTTCATATAGATGAAACTAATTTTTGGCTTA
AGTCAGAACAACTGGCCCCCAATTGAAGTCATATTTGTGGGGGGAAATGGCATACGCAATATTATATT
ATATTGGATATTTATGTTCACACAGGAATTTGGTTTACTGCTTTGTAAATAAAAGGGAAAACTCCGGG
TA

| | | | |
|---|---|---|---|
| NOV28h, CG55256-05 | SEQ ID NO:652 | 857 aa | MW at 95006.1 kD |
| Protein Sequence | | | |

+TL,1MGLPEPGPLRLLALLLLLLLLLLLRLQHLAAAAADPLLGGQGPAKECEKDQFQCRNERCIPSVWRCDE
DDDCLDHSDEDDCPKKTCADSDFTCDNGHCIHERWKCDGEEECPDGSDESEATCTLGTCRGDEFQCGD
GTCVLAIKHCNQEQDCPDGSDEAGCLQESPCEGPRRFQCKSGKRVDGGKVCDVQRDCRDWSDELLKVW
CGACLRPLAGLSLLPSPSWSPCPSPGLNECLHNNGGCSHICTDLKIGFECTCPAGFQLLDQKTCGDID
ECKDPDACSQICVNYKGYFKCECYPGYEMDLLTKNCKAAAGKSPSLIFTNRHEVRRIDLVKRNYSRLI
PMLKNVVALDVEVATNRIYWCDLSYRKIYSAYMDKASDPKEQEVLIDEQLHSPEGLAVDWVHKHIYWT
DSGNKTISVATVDGGRRRTLFSRNLSEPRAIAVDPLRGFMYWSDWGDQAKIEKSGLNGVDRQTLVSDN
IEWPNGITLDLLSQRLYWVDSKLHQLSSIDFSGGNRKTLISSTDFLSHPFGIAVFEDKVFWTDLENEA
IFSANRLNGLEISILAENLNNPHDIVIFHELKQPRAPDACELSVQPNGGCEYLCLPAPQISSHSPKYT
SVSVPRAPSISPSTLSPATSNHSQHYANEDSKMGSTVTAAVIGIIVPIVVIALLCMSGYLIWRNWKRK
NTKSMNFDNPVYRKTTEEEDEDELHIGRTAQIGHVYPAAISSFDRPLWAEPCLGETREPEDPAPALKE
LFVLPGEPRSQLHQLPKNPLSELPVVKSKRVALSLEDDGLP

| | | |
|---|---|---|
| NOV28i, CG55256-06 | SEQ ID NO:653 | 4180 bp |
| DNA Sequence | ORF Start: ATG at 163 | ORF Stop: TGA at 2704 |

ATGGCCTCCCCGAACGTATGGGCCTCCCCGAGCGTATGGGCCTCCCCGAGCGTATGGGCCTCCCCGAG
CGTATGGGCCTCACCCGAGCGTATGGGCCTCCCCGAGCGTATGGGCCTCCCCGAGCGTATGGGCCTCC
CCGAGCGTATGGGCCTCCCCGAGCGTATGGGCCTCCCCGAGCCGGGCCCTCTCCGGCTTCTGGCGCTG
CTGCTGCTGCTGCTGCTGCTGCTGCTGCTGCAGCTCCAGCATCTTGCGGCGGCAGCGGCTGATCCGCT
GCTCGGCGGCCAAGGGCCGGCCAAGGATTGCGAAAAGGACCAATTCCAGTGCCGGAACGAGCGCTGCA
TCCCCTCTGTGTGGAGATGCGACGAGGACGATGACTGCTTAGACCACAGCGACGAGGACGACTGCCCC
AAGAAGACCTGTGCAGACAGTGACTTCACCTGTGACAACGGCCACTGCATCCACGAACGGTGGAAGTG
TGACGGCGAGGAGGAGTGTCCTGATGGCTCCGATGAGTCCGAGGCCACTTGCACCAAGCAGGTGTGTC
CTGCAGAGAAGCTGAGCTGTGGACCCACCAGCCACAAGTGTGTACCTGCCTCGTGGCGCTGCGACGGG
GAGAAGGACTGCGAGGGTGGAGCGGATGAGGCCGGCTGTGCTACCTCACTGGGCACCTGCCGTGGGGA
CGAGTTCCAGTGTGGGGATGGGACATGTGTCCTTGCAATCAAGCACTGCAACCAGGAGCAGGACTGTC
AGGGGGCTGAACGAGTGTCTGCACAACAATGGCGGCTGCTCACACATCTGCACTGACCTCAAGATTGG
CTTTGAATGCACGTGCCCAGCAGGCTTCCAGCTCCTGGACCAGAAGACCTGTGGCGACATTGATGAGT
GCAAGGACCCAGATGCCTGCAGCCAGATCTGTGTCAATTACAAGGGCTATTTTAAGTGTGAGTGCTAC
CCTGGCTACGAGATGGACCTACTGACCAAGAACTGCAAGGCTGCTGCTGGAAAGAGCCCATCCCTAAT
CTTCACCAACCGGCACGAGGTGCGGAGGATCGACCTGGTGAAGCGGAACTATTCACGCCTCATCCCCA
TGCTCAAGAATGTCGTGGCACTAGATGTGGAAGTTGCCACCAATCGCATCTACTGGTGTGACCTCTCC
TACCGTAAGATCTATAGCGCCTACATGGACAAGGCCAGTGACCCGAAAGAGCAGGAGGTCCTCATTGA
CGAGCAGTTGCACTCTCCAGAGGGCCTGGCAGTGGACTGGGTCCACAAGCACATCTACTGGACTGACT
CGGGCAATAAGACCATCTCAGTGGCCACAGTTGATGGTGGCCGCCGACGCACTCTCTTCAGCCGTAAC
CTCAGTGAACCCCGGGCCATCGCTGTTGACCCCCTGCGAGGGTTCATGTATTGGTCTGACTGGGGGGA
CCAGGCCAAGATTGAGAAATCTGGGCTCAACGGTGTGGACCGGCAAACACTGGTGTCAGACAATATTG
AATGGCCCAACGGAATCACCCTGGATCTGCTGAGCCAGCGCTTGTACTGGGTAGACTCCAAGCTACAC
CAACTGTCCAGCATTGACTTCAGTGGAGGCAACAGAAAGACGCTGATCTCCTCCACTGACTTCCTGAG
CCACCCTTTTGGGATAGCTGTGTTTGAGGACAAGGTGTTCTGGACAGACCTGGAGAACGAGGCCATTT
TCAGTGCAAATCGGCTCAATGGCCTGGAAATCTCCATCCTGGCTGAGAACCTCAACAACCCACATGAC
ATTGTCATCTTCCATGAGCTGAAGCAGCCAAGAGCTCCAGATGCCTGTGAGCTGAGTGTCCAGCCTAA
TGGAGGCTGTGAATACCTGTGCCTTCCTGCTCCTCAGATCTCCAGCCACTCTCCCAAGTACACATGTG
CCTGTCCTGACACAATGTGGCTGGGTCCAGACATGAAGAGGTGCTACCGAGCACCTCAATCTACCTCA
ACTACGACGTTAGCTTCTACCATGACGAGGACAGTACCTGCCACCACAAGAGCCCCCGGGACCACCGT
CCACAGATCCACCTACCAGAACCACAGCACAGAGACACCAAGCCTGACAGCTGCAGTCCCAAGCTCAG
TTAGTGTCCCCAGGGCTCCCAGCATCAGCCCGTCTACCCTAAGCCCTGCAACCAGCAACCACTCCCAG
CACTATGCAAATGAAGACAGTAAGATGGGCTCAACAGTCACTGCCGCTGTTATCGGGATCATCGTGCC
CATAGTGGTGATAGCCCTCCTGTGCATGAGTGGATACCTGATCTGGAGAAACTGGAAGCGGAAGAACA
CCAAAAGCATGAATTTTGACAACCCAGTCTACAGGAAACAACAGAAGAAGAAGATGAAGATGAGCTC
CATATAGGGAGAACTGCTCAGATTGGCCATGTCTATCCTGCAGCAATCAGCAGCTTTGATCGCCCACT
GTGGGCAGAGCCCTGTCTTGGGGAGACCAGAGAACCGGAAGACCCAGCCCCTGCCCTCAAGGAGCTTT
TTGTCTTGCCGGGGGAACCAAGGTCACAGCTGCACCAACTCCCGAAGAACCCTCTTTCCGAGCTGCCT
GTCGTCAAATCCAAGCGAGTGGCATTAAGCCTTGAAGATGATGGACTACCCTGAGGATGGGATCACCC
TTCTATATATGGGTCTGTGTGAGTGTATGTGTGTGTGTGATTTTTTTTTTAAATTTATGTTGCGGAAA
GGTAACCACAAAGTTATGATGAACTGCAAACATCCAAAGGATGTGAGAGTTTTTCTATGTATAATGTT
TTATACACTTTTTAACTGGTTGCACTACCCATGAGGAATTCGTGGAATGGCTACTGCTGACTAACATG
ATGCACATAACCAAATGGGGGCCAATGGCACAGTACCTTACTCATCATTTAAAAACTATATTTACAGA
AGATGTTTGGTTGCTGGGGGGGCTTTTTTGGGTTTTGGGGCATTTGTTTTTTGTAAATAAGATGATTA
TGCTTTGTGGCTATCCATCAACATAAGTAAAAAAAAAAAAAAAACACTTCAACTCCCTCCCCCATTTA
GATTATTTATTAACATATTTTAAAAATCAGATGAGTTCTATAAATAATTTAGAGAAGTGAGAGTATTT
ATTTTTGGCATGTTTGGCCCACCACACAGACTCTGTGTGTGTATGTGTGTGTTTATATGTGTATGTGT
GTGACAGGAAAATCTGTAGAGAAGAGGCACATCTATGGCTACTGTTCAAATACATAAAGATAAATTTA
TTTTCACACAGTCCACAAGGGGTATATCTTGTAGTTTTCAGAAAAGCCTTTGGAAATCTGGATCAGGA
AATAGATACCATGGTTTGTGCAATTATGTAGTAAAAAAGGCAAATCTTTTCACCTCTGGCTATTCCTG
AGACCCCAGGAAGTCAGGAAAAGCCTTTCAGCTCACCCATGGCTGCTGTGACTCCTACCAGGGCTTTC
TTGGCTTTGGCGAAGGTCAGTGTACAGACATTCCATGGTACCAGAGTGCTCAGAAAGTCAAGATAGGA
AGGACACTTGGGTTGAATTCAGTAAGTTTCCTCTGAAGCACCCTGAAGGGTGCCATCCTTACAGAGCT
AAGTGGAGACGTTTCCAGATCAGCCCAAGTTTACTATAGAGACTGGCCCAGGCACTGAATGTCTAGGA
CATGCTGTGGATGAAGATAAAGATGGTGGAATAGGTTTTATCACATCTCTTATTTCTCTTTTCCCCTT
ACTCTCTACCATTTCCTTTATGTGGGGAAACATTTTAAGGTAATAAATAGGTTACTTACCATCATATC
TTCATATAGATGAAACTAATTTTTGGCTTAAGTCAGAACAACTGGCCCCCAATTGAAGTCATATTTGT
GGGGGGAAATGGCATACGCAATATTATATTATATTGGATATTTATGTTCACACAGGAATTTGGTTTAC

| | | | |
|---|---|---|---|
| NOV28i, CG55256-06 | SEQ ID NO:654 | 847 aa | MW at 93663.5 kD |
| Protein Sequence | | | |

+TL,1MGLPEPGPLRLLALLLLLLLLLLLQLQHLAAAAADPLLGGQGPAKDCEKDQFQCRNERCIPSVWRCDE
DDDCLDHSDEDDCPKKTCADSDFTCDNGHCIHERWKCDGEEECPDGSDESEATCTKQVCPAEKLSCGP
TSHKCVPASWRCDGEKDCEGGADEAGCATSLGTCRGDEFQCGDGTCVIAIKHCNQEQDCPDGSDEAGC
LQVPPTFLGNRRRPRGLNECLHNNGGCSHICTDLKIGFECTCPAGFQLLDQKTCGDIDECKDPDACSQ
ICVKGYFKCECYPGYEMDLLTKNGACKAAAGKSPSLIFTNRHEVRRIDLVKRNYSRLIPMLKNVVALD
TVDGGRRRTLFSRNLSEPRAIAVDPLRGFMYWSDWGDQAKIEKSGLNGVDRQTLVSDNIEWPNGITLD
LLSQRLYWVDSKLHQLSSIDFSGGNRKTLISSTDFLSHPFGIAVFEDKVFWTDLENEAIFSANRLNGL
EISILAENLNNPHDIVIFHELKQPRAPDACELSVQPNGGCEYLCLPAPQISSHSPKYTCACPDTMWLG
PDMKRCYRAPQSTSTTTLASTMTRTVPATTRAPGTTVHRSTYQNHSTETPSLTAAVPSSVSVPPAPSI
SPSTLSPATSNHSQHYANEDSKMGSTVTAAVIGIIVPIVVIALLCMSGYLIWRNWKRKNTKSMNFDNP
VYRKTTEEEDEDELHIGRTAQIGHVYPAAISSFDRPLWAEPCLGETREPEDPAPALKELFVLPGEPRS
QLHQLPAPLSELPWKSKRVALSLEDDGLP

| | | |
|---|---|---|
| NOV28, CG55256-08 | SEQ ID NO:655 | 2656 bp |
| DNA Sequence | ORF Start: ATG at 103 | ORF Stop: TGA at 2428 |

GCAGCGGCAACCCCGGCGCCGCGGCAAGGACTCGGAGGGCTGAGACGCGGCGGCGGCGGCGCGGGGAG
CGCGGGGCGCGGCGGCCGGAGCCCCGGGCCCGCCATGGGCCTCCCCGAGCCGGGCCCTCTCCGGCTTC
TGGCGCTGCTGCTGCTGCTGCTGCTGCTGCTGCTGCTGCGGCTCCAGCATCTTGCGGCGGCAGCGGCT
GATCCGCTGCTCGGCGGCCAAGGGCCGGCCAAGGAGTGCGAAAAGGACCAATTCCAGTGCCGGAACGA
GCGCTGCATCCCCTCTGTGTGGAGATGCGACGAGGACGATGACTGCTTAGACCACAGCGACGAGGACG
ACTGCCCCAAGAAGACCTGTGCAGACAGTGACTTCACCTGTGACAACGGCCACTGCATCCACGAACGG
TGGAAGTGTGACGGCGAGGAGGAGTGTCCTGATGGCTCCGATGAGTCCGAGGCCACTTGCACCAAGCA
GGTGTGTCCTGCAGAGAAGCTGAGCTGTGGACCCACCAGCCACAAGTGTGTACCTGCCTCGTGGCGCT
GCGACGGGGAGAAGGACTGCGAGGGTGGAGCGGATGAGGCCGGCTGTGCTACCTCACTGGGCACCTGC
CGTGGGGACGAGTTCCAGTGTGGGGATGGGACATGTGTCCTTGCAATCAAGCACTGCAACCAGGAGCA
GGACTGTCCAGATGGGAGTGATGAAGCTGGCTGCCTACAGGGGCTGAACGAGTGTCTGCACAACAATG
GCGGCTGCTCACACATCTGCACTGACCTCAAGATTGGCTTTGAATGCACGTGCCCAGCAGGCTTCCAG
CTCCTGGACCAGAAGACCTGTGGCGACATTGATGAGTGCAAGGACCCAGATGCCTGCAGCCAGATCTG
TGTCAATTACAAGGGCTATTTTAAGTGTGAGTGCTACCCTGGCTACGAGATGGACCTACTGACCAAGA
ACTGCAAGGCTGCTGCTGGCAAGAGCCCATCCCTAATCTTCACCAACCGGCACGAGGTGCGGAGGATC
GACCTGGTGAAGCGGAACTATTCACGCCTCATCCCCATGCTCAAGAATGTCGTGGCACTAGATGTGGA
AGTTGCCACCAATCGCATCTACTGGTGTGACCTCTCCTACCGTAAGATCTATAGCGCCTACATGGACA
AGGCCAGTGACCCGAAAGAGCAGGAGGTCCTCATTGACGAGCAGTTGCACTCTCCAGAGGGCCTGGCA
GTGGACTGGGTCCACAAGCACATCTACTGGACTGACTCGGGCAATAAGACCATCTCAGTGGCCACAGT
TGATGGTGGCCGCCGACGCACTCTCTTCAGCCGTAACCTCAGTGAACCCCGGGCCATCGCTGTTGGCC
CCCTGCGAGGGTTCATGTATTGGTCTGACTGGGGGGACCAGGCCAAGATTGAGAAATCTGGGCTCAAC
GGTGTGGACCGGCAAACACTGGTGTTAGACAATATTGAATGGCCCAACGGAATCACCCTGGATCTGCT
GAGCCAGCGCTTGTACTGGGTAGACTCCAAGCTACACCAACTGTCCAGCATTGACTTCAGTGGAGGCA
ACAGAAAGACGCTGATCTCCTCCACTGACTTCCTGAGCCACCCTTTTGGGATAGCTGTGTTTGAGGAC
AAGGTGTTCTGGACAGACCTGGAGAACGAGGCCATTTTCAGTGCAAATCGGCTCAATGGCCTGGAAAT
CTCCATCCTGGCTGAGAACCTCAACAACCCACATGACATTGTCATCTTCCATGAGCTGAAGCAGCCAA
GAGCTCCAGATGCCTGTGAGCTGAGTGTCCAGCCTAATGGAGGCTGTGAATACCTGTGCCTTCCTGCT
CCTCAGATCTCCAGCCACTCTCCCAAGTACACATGTGCCTGTCCTGACACAATGTGGCTGGGCCCAGA
CATGAAGAGGTGCTACCGAGCACCTCAATCTACCTCAACTACGACGTTAGCTTCTACCATGACGAGGA
CAGTACCTGCCACCACAAGAGCCCCCGGGACCACCGTCCACAGATCCACCTACCAGAACCACAGCACA
GAGACACCAAGCCTGACAGCTGCAGTCCCAAGCTCAGTTAGTGTCCCCAGGGCTCCCAGCATCAGCCC
GTCTACCCTAAGCCCTGCAACCAGCAACCACTCCCAGCACTATGCAAATGAAGACAGTAAGATGGGCT
CAACAGTCACTGCCGCTGTTATCGGGATCATCGTGCCCATAGTGGTGATAGCCCTCCTGTGCATGAGT
GGATACCTGATCTGGAGAAACTGGAAGCGGAAGAACACCAAAAGCATGAATTTTGACAACCCAGTCTA
CAGGAAAACAACAGAAGAAGAAGACGAAGATGAGCTCCATATAGGGAGAACTGCTCAGATTGGCCATG
TCTATCCTGCACGAGTGGCATTAAGCCTTGAAGATGATGGACTACCCTGAGGATGGGATCACCCCCTT
CGTGCCTCATGGAATTCAGTCCCATGCACTACACTCTGGAGGGTGTATGACTGGATGAATGGGTTTCT
ATATATGGGTCTGTGTGAGTGTATGTGTGTGTGTGATTTTTTTTTTAAATTTATGTTGCGGAAAGGTA
ACCACAAAGTTATGATGAACTGCAAACATCCAAAGGATGTGAGAGTTTTTCTATGTATAATGTTTTAT
ACAC

| | | | |
|---|---|---|---|
| NOV28, CG55256-08 | SEQ ID NO:656 | 775 aa | MW at 85584.3 kD |
| Protein Sequence | | | |

+TL,1MGLPEPGPLRLLALLLLLLLLLLLRLQHLAAAAADPLLGGQGPAKECEKDQFQCRNERCIPSVWRCDE
DDDCLDHSDEDDCPKKTCADSDFTCDNGHCIHERWKCDGEEECPDGSDESEATCTKQVCPAEKLSCGP
TSHKCVPASWRCDGEKDCEGGADEAGCATSLGTCRGDEFQCGDGTCVLAIKHCNQEQDCPDGSDEAGC
LQGLNECLHNNGGCSHICTDLKIGFECTCPAGFQLLDQKTCGDIDECKDPDACSQICVNYKGYFKCEC
YPGYEMDLLTKNCKAAAGKSPSLIFTNRHEVRRIDLVKRNYSRLIPMLKNVVALDVEVATNRIYWCDL
SYRKIYSAYMDKASDPKEQEVLIDEQLHSPEGLAVDWVHKHIYWTDSGNKTISVATVDGGRRRTLFSR
NLSEPRAIAVGPLRGFMYWSDWGDQAKIEKSGLNGVDRQTLVLDNIEWPNGITLDLLSQRLYWVDSKL
HQLSSIDFSGGNRKTLISSTDFLSHPFGIAVFEDKVFWTDLENEAIFSANRLNGLEISILAENLNNPH
DIVIFHELKQPRAPDACELSVQPNGGCEYLCLPAPQISSHSPKYTCACPDTMWLGPDMKRCYPAPQST
STTTLASTMTRTVPATTRAPGTTVHRSTYQNHSTETPSLTAAVPSSVSVPRAPSISPSTLSPATSNHS
QHYANEDSKMGSTVTAAVIGIIVPIVVIALLCMSGYLIWRNWKRKNTKSNNFDNPVYRKTTEEEDEDE
LHIGRTAQIGHVYPARVALSLEDDGLP

| | | |
|---|---|---|
| NOV28k, CG55256-09 | SEQ ID NO:657 | 2623 bp |
| DNA Sequence | ORF Start: ATG at 103 | ORF Stop: TGA at 2605 |

GCAGCGGCAACCCCGGCGCCGCGGCAAGGACTCGGAGGGCTGAGACGCGGCGGCGGCGGCGCGGGGAG
CGCGGGGCGCGGCGGCCGGAGCCCCGGGCCCGCCATGGGCCTCCCCGAGCCGGGCCCTCTCCGGCTTC
TGGCGCTGCTGCTGCTGCTGCTGCTGCTGCTGCTGCTGCGGCTCCAGCATCTTGCGGCGGCAGCGGCT
GATCCGCTGCTCGGCGGCCAAGGGCCGGCCAAGGATTGCGAAAAGGACCAATTCCAGTGCCGGAACGA
GCGCTGCATCCCCTCTGTGTGGAGATGCGACGAGGACGATGACTGCTTAGACCACAGCGACGAGGACG
ACTGCCCCAAGAAGACCTGTGCAGACAGTGACTTCACCTGTGACAACGGCCACTGCATCCACGAACGG
TGGAAGTGTGACGGCGAGGAGGAGTGTCCTGATGGCTCCGATGAGTCCGAGGCCACTTGCACCAAGCA
GGTGTGTCCTGCAGAGAAGCTGAGCTGTGGACCCACCAGCCACAAGTGTGTACCTGCCTCGTGGCGCT
GCGACGGGGAGAAGGACTGCGAGGGTGGAGCGGATGAGGCCGGCTGTGCTACCTCACTGGGCACCTGC
CGTGGGGACGAGTTCCAGTGTGGGGATGGGACATGTGTCCTTGCAATCAAGCACTGCAACCAGGAGCA
GGACTGTCCAGATGGGAGTGATGAAGCTGGCTGCCTACAGGGGCTGAACGAGTGTCTGCACAACAATG
GCGGCTGCTCACACATCTGCACTGACCTCAAGATTGGCTTTGAATGCACGTGCCCAGCAGGCTTCCAG
CTCCTGGACCAGAAGACCTGTGGCGACATTGATGAGTGCAAGGACCCAGATGCCTGCAGCCAGATCTG
TGTCMATTACAAGGGCTATTTTAAGTGTGAGTGCTACCCTGGCTACGAGATGGACCTACTGACCAAGA
ACTGCAAGGCTGCTGCTGGCAAGAGCCCATCCCTAATCTTCACCAACCGGCACGAGGTGCGGAGGATC
GACCTGGTGAAGCGGAACTATTCACGCCTCATCCCCATGCTCAAGAATGTCGTGGCACTAGATGTGGA
AGTTGCCACCAATCGCATCTACTGGTGTGACCTCTCCTACCGTAAGATCTATAGCGCCTACATGGACA
AGGCCAGTGACCCGAAAGAGCAGGAGGTCCTCATTGACGAGCAGTTGCACTCTCCAGAGGGCCTGGCA
GTGGACTGGGTCCACAAGCACATCTACTGGACTGACTCGGGCAATAAGACCATCTCAGTGGCCACAGT
TGATGGTGGCCGCCGACGCACTCTCTTCAGCCGTAACCTCAGTGAACCCCGGGCCATCGCTGTTGACC
CCCTGCGAGGGTTCATGTATTGGTCTGACTGGGGGGACCAGGCCAAGATTGAGAAATCTGGGCTCAAC
GGTGTGGACCGGCAAACACTGGTGTCAGACAATATTGAATGGCCCAACGGAATCACCCTGGATCTGCT
GAGCCAGCGCTTGTACTGGGTAGACTCCAAGCTACACCAACTGTCCAGCATTGACTTCAGTGGAGGCA
ACAGAAAGACGCTGATCTCCTCCACTGACTTCCTGAGCCACCCTTTTGGGATAGCTGTGTTTGAGGAC
CTCCATCCTGGCTGAGAACCTCAACAACCCACATGACATTGTCATCTTCCATGAGCTGAAGCAGCCAA
CCTCAGATCTCCAGCCACTCTCCCAAGTACACATGTGCCTGTCCTGACACAATGTGGCTGGGTCCAGA
CATGAAGAGGTGCTACCGAGCACCTCAATCTACCTCAACTACGACGTTAGCTTCTACCATGACGAGGA
CAGTACCTGCCACCACAAGAGCCCCCGGGACCACCGTCCACAGATCCACCTACCAGAACCACAGCACA
GAGACACCAAGCCTGACAGCTGCAGTCCCAAGCTCAGTTAGTGTCCCCAGGGCTCCCAGCATCAGCCC
GTCTACCCTAAGCCCTGCAACCAGCAACCACTCCCAGCACTATGCAAATGAAGACAGTAAGATGGGCT
CAACAGTCACTGCCGCTGTTATCGGGATCATCGTGCCCATAGTGGTGATAGCCCTCCTGTGCATGAGT
GGATACCTGATCTGGAGAAACTGGAAGCGGAAGAACACCAAAAGCATGAATTTTGCCAACCCAGTCTA
CAGGAAAACAACAGAAGAAGAAGACGAAGATGAGCTCCATATAGGGAGAACTGCTCAGATTGGCCATG
TCTATCCTGCAGCAATCAGCAGCTTTGATCGCCCACTGTGGGCAGAGCCCTGTCTTGGGGAGACCAGA
GAACCGGAAGACCCAGCCCCTGCCCTCAAGGAGCTTTTTGTCTTGCCGGGGGAACCAAGGTCACAGCT
GCACCAACTCCCGAAGAACCCTCTTTCCGAGCTGCCTGTCGTCAAATCCAAGCGAGTGGCATTAAGCC
TTGAAGATGATGGACTACCCTGAGGATGGGATCACCCCC

| | | | |
|---|---|---|---|
| NOV28k, CG55256-09 | SEQ ID NO:658 | 834 aa | MW at 92099.7 kD |
| Protein Sequence | | | |

MGLPEPGPLRLLALLLLLLLLLLLRLQHLAAAAADPLLGGQGPAKDCEKDQFQCRNERCIPSVWRCDE
DDDCLDHSDEDDCPKKTCADSDFTCDNGHCIHERWKCDGEEECPDGSDESEATCTKQVCPAEKLSCGP
TSHKCVPASWRCDGEKDCEGGADEAGCATSLGTCRGDEFQCGDGTCVLAIKHCNQEQDCPDGSDEAGC
LQGLNECLHNNGGCSHICTDLKIGFECTCPAGFQLLDQKTCGDIDECKDPDACSQICVNYKGYFKCEC
YPGYEMDLLTKNCKAAAGKSPSLIFTNRHEVRRIDLVKRNYSRLIPMLKNVVALDVEVATNRIYWCDL
SYRKIYSAYMDKASDPKEQEVLIDEQLHSPEGLAVDWVHKHIYWTDSGNKTISVATVDGGRRRTLFSR
NLSEPRAIAVDPLRGFMYWSDWGDQAKIEKSGLNGVDRQTLVSDNIEWPNGITLDLLSQRLYWVDSKL
HQLSSIDFSGGNRKTLISSTDFLSHPFGIAVFEDKVFATDLENEAIFSANRLNGLEISILAENLNNPH
DIVIFHELKQPRAPDACELSVQPNGGCEYLCLPAPQISSHSPKYTCACPDTMWLGPDMKRCYRAPQST
STTTLASTMTRTVPATTRAPGTTVHRSTYQNHSTETPSLTAAVPSSVSVPRAPSISPSTLSPATSNHS
QHYANEDSKMGSTVTAAVIGIIVPIVVIALLCMSGYLIWRNWKRKNTKSMNFANPVYRKTTEEEDEDE
LHIGRTAQIGHVYPAAISSFDRPLWAEPCLGETREPEDPAPALKELFVLPGEPRSQLHQLPKNPLSEL
PVVKSKRVALSLEDDGLP

| | | |
|---|---|---|
| NOV281, CG55256-11 | SEQ ID NO:659 | 4607 bp |
| DNA Sequence | ORF Start: ATG at 237 | ORF Stop: TGA at 3126 |

GCTGGCGGCGGCCGCCCAGGGCCGGGGCCGCGCGCCCAGCCTGAGCCCGCCCCGCCGCCGAGCGTCAC
CGAACCTGCTTGAAATGCAGCCGAGGAGCCGGGGCGGGCGGCAGCGGCGGCGGCGGCGGCGGCGGGGG
CAGCGGCAACCCCGGCGCCGCGGCAAGGACTCGGAGGGCTGAGACGCGGCGGCGGCGGCGCGGGGAGC
GCGGGGCGCGGCGGCCGGAGCCCGGGCCCGCCATGGGCCTCCCCGAGCCGGGCCCTCTCCGGCTTCTG
GCGCTGCTGCTGCTGCTGCTGCTGCTGCTGCTGCTGCGGCTCCAGCATCTTGCGGCGGCAGCGGCTGA
TCCGCTGCTCGGCGGCCAAGGGCCGGCCAAGGAGTGCGAAAAGGACCAATTCCAGTGCCGGAACGAGC
GCTGCATCCCCTCTGTGTGGAGATGCGACGAGGACGATGACTGCTTAGACCACAGCGACGAGGACGAC
TGCCCCAAGAAGACCTGTGCAGACAGTGACTTCACCTGTGACAACGGCCACTGCATCCACGAACGGTG
GAAGTGTGACGGCGAGGAGGAGTGTCCTGATGGCTCCGATGAGTCCGAGGCCACTTGCACCAAGCAGG
TGTGTCCTGCAGAGAAGCTGAGCTGTGGACCCACCAGCCACAAGTGTGTACCTGCCTCGTGGCGCTGC
GACGGGGAGAAGGACTGCGAGGGTGGAGCGGATGAGGCCGGCTGTGCTACCTTGTGCGCCCCGCACGA
GTTCCAGTGCGGCAACCGCTCGTGCCTGGCCGCCGTGTTCGTGTGCGACGGCGATGACGACTGTGGTG
ACGGCAGCGATGAGCGCGGCTGTGCAGACCCGGCCTGCGGGCCCCGCGAGTTCCGCTGCGGCGGCGAT
GGCGGCGGCGCCTGCATCCCGGAGCGCTGGGTCTGCGACCGCCAGTTTGACTGCGAGGACCGCTCGGA
CGAGGCAGCCGAGCTCTGCGGCCGCCCGGGCCCCGGGGCCACGTCCGCGCCCGCCGCCTGCGCCACCG
TCTCCCAGTTCGCCTGCCGCAGCGGCGAGTGCGTGCACCTGGGCTGGCGCTGCGACGGCGACCGCGAC
TGCAAAGACAAATCGGACGAGGCCGACTGCCCACTGGGCACCTGCCGTGGGGACGAGTTCCAGTGTGG
GGATGGGACATGTGTCCTTGCAATCAAGCACTGCAACCAGGAGCAGGACTGTCCAGATGGGAGTGATG
AAGCTGGCTGCCTACAGGGGCTGAACGAGTGTCTGCACAACAATGGCGGCTGCTCACACATCTGCACT
GACCTCAAGATTGGCTTTGAATGCACGTGCCCAGCAGGCTTCCAGCTCCTGGACCAGAAGACCTGTGG
CGACATTGATGAGTGCAAGGACCCAGATGCCTGCAGCCAGATCTGTGTCAATTACAAGGGCTATTTTA
AGTGTGAGTGCTACCCTGGCTACGAGATGGACCTACTGACCAAGAACTGCAAGGCTGCTGGTGGAAAG
AGCCCATCCCTAATCTTCACCAACCGGTACGAGGTGCGGAGGATCGACCTGGTGAAGCGGAACTATTC
ACGCCTCATCCCCATGCTCAAGAATGTCGTGGCACTAGATGTGGAAGTTGCCACCAATCGCATCTACT
GGTGTGACCTCTCCTACCGTAAGATCTATAGCGCCTACATGGACAAGGCCAGTGACCcGAAAGAGcAG
GAGGTCCTCATTGACGAGCAGTTGCACTCTCCAGAGGGCCTGGCAGTGGACTGGGTCCACAAGCACAT
CTACTGGACTGACTCGGGCAATAAGACCATCTCAGTGGCCACAGTTGATGGTGGCCGCCGACGCACTC
TCTTCAGCCGTAACCTCAGTGAACCCCGGGCCATCGCTGTTGACCCCCTGCGAGGGTTCATGTATTGG
TCTGACTGGGGGGACCAGGCCAAGATTGAGAAATCTGGGCTCAACGGTGTGGACCGGCAAACACTGGT
GTCAGACAATATTGAATGGCCCAACGGAATCACCCTGGATCTGCTGAGCCAGCGCTTGTACTGGGTAG
ACTCCAAGCTACACCAACTGTCCAGCATTGACTTCAGTGGAGGCAACAGAAAGACGCTGATCTCCTCC
ACTGACTTCCTGAGCCACCCTTTTGGGATAGCTGTGTTTGAGGACAAGGTGTTCTGGACAGACCTGGA
GAACGAGGCCATTTTCAGTGCAAATCGGCTCAATGGCCTGGAAATCTCCATCCTGGCTGAGAACCTCA
ACAACCCACATGACATTGTCATCTTCCATGAGCTGAAGCAGCCAAGAGCTCCAGATGCCTGTGAGCTG
AGTGTCCAGCCTAATGGAGGCTGTGAATACCTGTGCCTTCCTGCTCCTCAGATCTCCAGCCACTCTCC
CAAGTACACATGTGCCTGTCCTGACACAATGTGGCTGGGTCCAGACATGAAGAGGTGCTACCGAGCAC
CTCAATCTACCTCAACTACGACGTTAGCTTCTACCATGACGAGGACAGTACCTGCCACCACAAGAGCC
CCCGGGACCACCGTCCACAGATCCACCTACCAGAACCACAGCACAGAGACACCAAGCCTGACAGCTGC
AGTCCCAAGCTCAGTTAGTGTCCCCAGGGCTCCCAGCATCAGCCCGTCTACCCTAAGCCCTGCAACCA
GGGATCATCGTGCCCATAGTGGTGATAGCCCTCCTGTGCATGAGTGGATACCTGATCTGGAGAAACTG
GAAGCGGAAGAACACCAAAAGCATGAATTTTGACAACCCAGTCTACAGGAAAACAACAGAAGAAGAAG
ATGAAGATGAGCTCCATATAGGGAGAACTGCTCAGATTGGCCATGTCTATCCTGCAGCAATCAGCAGC
TTTGATCGCCCACTGTGGGCAGAGCCCTGTCTTGGGGAGACCAGAGAACCGGAAGACCCAGCCCCTGC
CCTCAAGGAGCTTTTTGTCTTGCCGGGGGAACCAAGGTCACAGCTGCACCAACTCCCGAAGAACCCTC
TTTCCGAGCTGCCTGTCGTCAAATCCAAGCGAGTGGCATTAAGCCTTGAAGATGATGGACTACCCTGA
GGATGGGATCACCCCCTTCGTGCCTCATGGAATTCAGTCCCATGCACTACACTCTGGATGGTGTATGA
CTGGATGAATGGGTTTCTATATATGGGTCTGTGTGAGTGTATGTGTGTGTGTGATTTTTTTTTTAAAT
TTATGTTGCGGAAAGGTAACCACAAAGTTATGATGAACTGCAAACATCCAAAGGATGTGAGAGTTTTT
CTATGTATAATGTTTTATACACTTTTTAACTGGTTGCACTACCCATGAGGAATTCGTGGAATGGCTAC
TGCTGACTAACATGATGCACATAACCAAATGGGGGCCAATGGCACAGTACCTTACTCATCATTTAAAA
ACTATATTTACAGAAGATGTTTGGTTGCTGGGGGGGCTTTTTTGGGTTTTGGGGCATTTGTTTTTTGT
AAATAAGATGATTATGCTTTGTGGCTATCCATCAACATAAGTAAAAAAAAAAAAAAAACACTTCAACT
CCCTCCCCCATTTAGATTATTTATTAACATATTTTAAAAATCAGATGAGTTCTATAAATAATTTAGAG
AAGTGAGAGTATTTATTTTTGGCATGTTTGGCCCACCACACAGACTCTGTGTGTGTATGTGTGTGTTT
ATATGTGTATGTGTGTGACAGGAAAATCTGTAGAGAAGAGGCACATCTATGGCTACTGTTCAAATACA
TAAAGATAAATTTATTTTCACACAGTCCACAAGGGGTATATCTTGTAGTTTTCAGAAAAGCCTTTGGA
AATCTGGATCAGGAAATAGATACCATGGTTTGTGCAATTATGTAGTAAAAAAGGCAAATCTTTTCACC
TCTGGCTATTCCTGAGACCCCAGGAAGTCAGGAAAAGCCTTTCAGCTCACCCATGGCTGCTGTGACTC
CTACCAGGGCTTTCTTGGCTTTGGCCAAGGTCAGTGTACAGACATTCCATGGTACCAGAGTGCTCAGA
AAGTCAAGATAGGATATGCCTGACCCTCAGCTACTCCTTGTTTTAAAGTTCAGCTCTTTGAGTAACTT
CTTCAATTTCTTTCAGGACACTTGGGTTGAATTCAGTAAGTTTCCTCTGAAGCACCCTGAAGGGTGCC
ATCCTTACAGAGCTAAGTGGAGACGTTTCCAGATCAGCCCAAGTTTACTATAGAGACTGGCCCAGGCA
CTGAATGTCTAGGACATGCTGTGGATGAAGATAAAGATGGTGGAATAGGTTTTATCACATCTCTTATT
TCTCTTTTCCCCTTACTCTCTACCATTTCCTTTATGTGGGGAAACATTTTAAGGTAATAAATAGGTTA
CTTACCATCATATGTTCATATAGATGAAACTAATTTTTGGCTTAAGTCAGAACAACTGGCCCCCAATT
GAAGTCATATTTGTGGGGGGAAATGGCATACGCAATATTATATTATATTGGATATTTATGTTCACACA
GGAATTTGGTTTACTGCTTTGTAAATAAAAGGGAAAACTCCGGGTATATGT

| | | | | |
|---|---|---|---|---|
| NOV281, CG55256-11 | SEQ ID NO:660 | 963 aa | MW at 105714.3 kD | I |
| Protein Sequence | | | | |

MGLPEPGPLRLLALLLLLLLLLLLRLQHLAAAAADPLLGGQGPAKECEKDQFQCRNERCIPSVWRCDE
DDDCLDHSDEDDCPKKTCADSDFTCDNGHCIHERWKCDGEEECPDGSDESEATCTKQVCPAEKLSCGP
TSHKCVPASWRCDGEKDCEGGADEAGCATLCAPHEFQCGNRSCLAAVFVCDGDDDCGDGSDERGCADP
ACGPREFRCGGDGGGACIPERWVCDRQFDCEDRSDEAAELCGRPGPGATSAPAACATVSQFACRSGEC
VHLGWRCDGDRDCKDKSDEADCPLGTCRGDEFQCGDGTCVLAIKHCNQEQDCPDGSDEAGCLQGLNEC
LHNNGGCSHICTDLKIGFECTCPAGFQLLDQKTCGDIDECKDPDACSQICVNYKGYFKCECYPGYEMD
LLTKNCKAAGGKSPSLIFTNRYEVRRIDLVKRNYSRLIPMLKNVVALDVEVATNRIYWCDLSYRKIYS
AYMDKASDPKEQEVLIDEQLHSPEGLAVDWVHKHIYWTDSGNKTISVATVDGGRRRTLFSRNLSEPRA
IAVDPLRGFMYWSDWGDQAKIEKSGLNGVDRQTLVSDNIEWPNGITLDLLSQRLYWVDSKLHQLSSID
FSGGNRKTLISSTDFLSHPFGIAVFEDKVFWTDLENEAIFSANRLNGLEISILAENLNNPHDIVIFHE
LKQPRAPDACELSVQPNGGCEYLCLPAPQISSHSPKYTCACPDTMWLGPDMKRCYPAPQSTSTTTLAS
TMTRTVPATTRAPGTTVHRSTYQNHSTETPSLTAAVPSSVSVPAAPSISPSTLSPATSNHSQHYANED
SKMGSTVTAAVIGIIVPIVVIALLCMSGYLIWRWKRKNTKSMNFDNPVYR1CTTEEEDEDELHIGRTA
QIGHVYPAAISSFDRPLWAEPCLGETREPEDPAPALKELFVLPGEPRSQLHQLPKNPLSELPVVKSKR
VALSLEDDGLP

| | | |
|---|---|---|
| NOV28m, SNP13382497 of | SEQ ID NO:661 | 4294 bp |
| CG55256-07, | ORF Start: ATG at 103 | ORF Stop: TGA at 2818 |
| DNA Sequence | SNP Pos: 2550 | SNP Change: T to C |

GCAGCGGCAACCCCGGCGCCGCGGCAAGGACTCGGAGGGCTGAGACGCGGCGGCGGCGGCGCGGGGAG
CGCGGGGCGCGGCGGCCGGAGCCCCGGGCCCGCCATGGGCCTCCCCGAGCCGGGCCCTCTCCGGCTTC
TGGCGCTGCTGCTGCTGCTGCTGCTGCTGCTGCTGCTGCGGCTCCAGCATCTTGCGGCGGCAGCGGCT
ACTGCCCCAAGAAGACCTGTGCAGACAGTGACTTCACCTGTGACAACGGCCACTGCATCCACGAACGG
TGGAAGTGTGACGGCGAGGAGGAGTGTCCTGATGGCTCCGATGAGTCCGAGGCCACTTGCACACTGGG
CACCTGCCGTGGGGACGAGTTCCAGTGTGGGGATGGGACATGTGTCCTTGCAATCAAGCACTGCAACC
AGGAGCAGGACTGTCCAGATGGGAGTGATGAAGCTGGCTGCCTACAGGAGTCACCCTGTGAGGGTCCC
CGCAGATTTCAGTGTAAGAGTGGCAAGCGCGTGGACGGCGGGAAAGTGTGTGATGTGCAGAGGGACTG
CCGGGACTGGTCGGATGAGCTTCTGAAAGTGTGGTGCGGTGCCTGTCTACGCCCACTGGCTGGACTCA
GTCTCCTACCATCCCCCTCCTGGTATCTAGGCTCAAGGCCCTCCAGTGCCCCCTGCCCTGACACTTTC
TGCTCTGACCCTCTCTTTGGATTCATGTGCCGTCCTATGGCTTCACATGGGGCTTTTCGCCCCCAGGC
CTCTGGTCTACATCTCTACAAAGTGCTAAGAGCTTGTCCATCCCAGAGGAGGCTGAACGAGTGTCTGC
ACAACAATGGCGGCTGCTCACACATCTGCACTGACCTCAAGATTGGCTTTGAATGCACGTGCCCAGCA
GGCTTCCAGCTCCTGGACCAGAAGACCTGTGGCGACATTGATGAGTGCAAGGACCCAGATGCCTGCAG
CCAGATCTGTGTCAATTACAAGGGCTATTTTAAGTGTGAGTGCTACCCTGGCTACGAGATGGACCTAC
TGACCAAGAACTGCCAAGCTGCTGCTGGAAAGAGCCCATCCCTAATCTTCACCAACCGGTACGAGGTG
CGGAGGATCGACCTGGTGAAGCGGAACTATTCACGCCTCATCCCCATGCTCAAGAATGTCGTGGCACT
ACATGGACAAGGCCAGTGACCCGAAAGAGCAGGAGGTCCTCATTGACGAGCAGTTGCACTCTCCAGAG
GGCCTGGCAGTGGACTGGGTCCACAAGCACATCTACTGGACTGACTCGGGCAATAAGACCATCTCAGT
GGGCTCAACGGTGTGGACCGGCAAACACTGGTGTCAGACAATATTGAATGGCCCAACGGAATCACCCT
GGATCTGCTGAGCCAGCGCTTGTACTGGGTAGACTCCAAGCTACACCAACTGTCCAGCATTGACTTCA
GTGGAGGCAACAGAAAGACGCTGATCTCCTCCACTGACTTCCTGAGCCACCCTTTTGGGATAGCTGTG
TTTGAGGACAAGGTGTTCTGGACAGACCTGGAGAACGAGGCCATTTTCAGTGCAAATCGGCTCAATGG
CCTGGAAATCTCCATCCTGGCTGAGAACCTCAACAACCCACATGACATTGTCATCTTCCATGAGCTGA
AGCAGCCAAGACCTCCAGATGCCTGTGAGCTGAGTGTCCAGCCTAATGGAGGCTGTGAATACCTGTGC
CTTCCTGCTCCTCAGATCTCCAGCCACTCTCCCAAGTACACATGTGCCTGTCCTGACACAATGTGGCT
GGGTCCAGACATGAAGAGGTGCTACCGAGCACCTCAATCTACCTCAACTACGACGTTAGCTTCTACCA
TGACGAGGACAGTACCTGCCACCACAAGAGCCCCCGGGACCACCGTCCACAGATCCACCTACCAGAAC
CACAGCACAGAGACACCAAGCCTGACAGCTGCAGTCCCAAGCTCAGTTAGTGTCCCCAGGGCTCCCAG
CATCAGCCCGTCTACCCTAAGCCCTGCAACCAGCAACCACTCCCAGCACTATGCAAATGAAGACAGTA
AGATGGGCTCAACAGTCACTGCCGCTGTTATCGGGATCATCGTGCCCATAGTGGTGATAGCCCTCCTG
TGCATGAGTGGATACCTGATCTGGAGAAACTGGAAGCGGAAGAACACCAAAAGCATGAATTTTGACAA
CCCAGTCTACAGGAAAACAACAGAAGAAGAAGACGAAGATGAGCTCCATATAGGGAGAACTGCTCAGA
TTGGCCATGTCTATCCTGCAGCAATCAGCAGCTTTGATCGCCCACTGTGGGCAGAGCCCTGTCTTGGG
GAGACCAGAGAACCGGAAGACCCAGCCCCTGCCCTCAAGGAGCTTTTTGTCTTGCCGGGGGAACCAAG
GTCACAGCTGCACCAACTCCCGAAGAACCCTCTTTCCGAGCTGCCTGTCGTCAAATCCAAGCGAGTGG
CATTAAGCCTTGAAGATGATGGACTACCCTGAGGATGGGATCACCCCCTTCGTGCCTCATGGAATTCA
GTCCCATGCACTACACTCTGGATGGTGTATGACTGGATGAATGGGTTTCTATATATGGGTCTGTGTGA
GTGTATGTGTGTGTGTGATTTTTTTTTTAAATTTATGTTGCGGAAAGGTAACCACAAAGTTATGATGA
ACTGCAAACATCCAAAGGATGTGAGAGTTTTTCTATGTATAATGTTTTATACACTTTTTAACTGGTTG
CACTACCCATGAGGAATTCGTGGAATGGCTACTGCTGACTAACATGATGCACATAACCAAATGGGGGC
CAATGGCACAGTACCTTACTCATCATTTAAAAACTATATTTACAGAAGATGTTTGGTTGCTGGGGGGG
CTTTTTTGGGTTTTGGGGCATTTGTTTTTTGTAAATAAGATGATTATGCTTTGTGGCTATCCATCAAC
ATAAGTAAAAAAAAAAAAAAAACACTTCAACTCCCTCCCCCATTTAGATTATTTATTAACATATTTTA
AAAATCAGATGAGTTCTATAAATAATTTAGAGAAGTGAGAGTATTTATTTTTGGCATGTTTGGCCCAC
CACACAGACTCTGTGTGTGTATGTGTGTGTTTATATGTGTATGTGTGTGACAGGAAAATCTGTAGAGA
AGAGGCACATCTATGGCTACTGTTCAAATACATAAAGATAAATTTATTTTCACACAGTCCACAAGGGG
TATATCTTGTAGTTTTCAGAAAAGCCTTTGGAAATCTGGATCAGGAAATAGATACCATGGTTTGTGCA
ATTATGTAGTAAAAAAGGCAAATCTTTTCACCTCTGGCTATTCCTGAGACCCCAGGAAGTCAGGAAAA
GCCTTTCAGCTCACCCATGGCTGCTGTGACTCCTACCAGGGCTTTCTTGGCTTTGGCGAAGGTCAGTG
TACAGACATTCCATGGTACCAGAGTGCTCAGAAAGTCAAGATAGGATATGCCTCACCCTCAGCTACTC
CTTGTTTTAAAGTTCAGCTCTTTGAGTAACTTCTTCAATTTCTTTCAGGACACTTGGGTTGAATTCAG
TAAGTTTCCTCTGAAGCACCCTGAAGGGTGCCAATCCTTACAGAGCTAAGTAAGACGTTTCCAGATCA
GCCCAAGTTTACTATAGAGACTGGCCCAGGCACTGAATGTCTAGGACATGCTGTGGATGAAGATAAAG
ATGGTGGAATAGGTTTTATCACATCTCTTATTTCTCTTTTCCCCTTACTCTCTACCATTTCCTTTATG
TGGGGAAACATTTTAAGGTAATAAATAGGTTACTTACCATCATATGTTCATATAGATGAAACTAATTT
TTGGCTTAAGTCAGAACAACTGGCCCCCAATTGAAGTCATATTTGTGGGGGGAAATGGCATACGCAAT
ATTATATTATATTGGATATTTATGTTCACACAGGAATTTGGTTTACTGCTTTGTAAATAAAAGGGAAA
ACTCCGGGTA

| | | | |
|---|---|---|---|
| NOV28m, SNP13382497 of | SEQ ID NO:662 | 905 aa | MW at 100461.4 kD |
| CG55256-07, Protein | SNP Pos: 816 | | SNP Change: Asp to Asp |
| Sequence | | | |

MGLPEPGPLRLLALLLLLLLLLLLRLQHLAAAAADPLLGGQGPAKECEKDQFQCRNERCIPSVWRCDE
DDDCLDHSDEDDCPKKTCADSDFTCDNGHCIHERWKCDGEEECPDGSDESEATCTLGTCRGDEFQCGD
GTCVNAIKHCNQEQDCPDGSDEAGCLQESPCEGPRRFQCKSGKRVDGGKVCDVQRDCRDWSDELLKVW
CGACLRPLAGLSLLPSPSWYLGSRPSSAPCPDTFCSDPLFGFMCRPMASHGAFRPQASGLHLYKVLRA
CPSQRRLNECLHNNGGCSHICTDLKIGFECTCPAGFQLLDQKTCGDIDECKDPDACSQICVNYKGYFK
CECYPGYEMDLLTKNCQAAAGKSPSLIFTNRYEVRRIDLVKRNYSRLIPMLKNVVALDVEVATNRIYW
CDLSYRKIYSAYMDKASDPKEQEVLIDEQLHSPEGLAVDWVHKHIYWTDSGNKTISVATVDGGRRRTL
FSRNLSEPRAIAVDPLRGFMYWSDWGDQAKIEKSGLNGVDRQTLVSDNIEWPNGITLDLLSQRLYWVD
SKLHQLSSIDFSGGNRKTLISSTDFLSHPFGIAVFEDKVFWTDLENEAIFSANRLNGLEISILAENLN
NPHDIVIFHELKQPAAPDACELSVQPNGGCEYLCLPAPQISSHSPKYTCACPDTMWLGPDMKRCYRAP
QSTSTTTLASTMTRTVPATTRAPGTTVHRSTYQNHSTETPSLTAAVPSSVSVPRAPSISPSTLSPATS
NHSQHYANEDSKMGSTVTAAVIGIIVPIVVIALLCMSGYLIWRNWKRKNTKSMNFDNPVYRKTTEEED
EDELHIGRTAQIGHVYPAAISSFDRPLWAEPCLGETREPEDPAPALKELFVLPGEPRSQLHQLPKNPL
SELPVVKSKRVALSLEDDGLP

| | | |
|---|---|---|
| NOV28n, SNP13382498 of | SEQ ID NO:663 | 4294 bp |
| CG55256-07, | ORF Start: ATG at 103 | ORF Stop: TGA at 2818 |
| DNA Sequence | SNP Pos: 2783 | SNP Change: G to A |

GCAGCGGCAACCCCGGCGCCGCGGCAAGGACTCGGAGGGCTGAGACGAGGCGGCGGCGGCGCGGGGAG
CGCGGGGCGCGGCGGCCGGAGCCCCGGGCCCGCCATGGGCCTCCCCGAGCCGGGCCCTCTCCGGCTTC
TGGCGCTGCTGCTGCTGCTGCTGCTGCTGCTGCTGCTGCGGCTCCAGCATGTTGCGGCGGCAGCGGCT
GATCCGCTGCTCGGCGGCCAAGGGCCGGCCAAGGAGTGCGAAAAGGACCAATTCCAGTGCCGGAACGA
GCGCTGCATCCCCTCTGTGTGGAGATGCGACGAGGACGATGACTGCTTAGACCACACAGACGAGGACG
ACTGCCCCAAGAAGACCTGTGCAGACAGTGACTTCACCTGTGACAACGGCCACTGCATCCACGAACGG
TGGAAGTGTGACGGCGAGGAGGAGTGTCCTGATGGCTCCGATGAGTCCGAGGCCACTTGCACACTGGG
CACCTGCCGTGGGGACGAGTTCCAGTGTGGGGATGGGACATGTGTCCTTGCATCAAAGCACTGCAACC
AGGAGCAGGACTGTCCAGATGGGAGTGATGAAGCTGGCTGCCTACAGGAGTCACCCTGTGAGGGTCCC
CGCAGATTTCAGTGTAAGAGTGGCAAGCGCGTGGACGGCGGGAAAGTGTGTGATGTGCAGAGGGACTG
CCGGGACTGGTCGGATGAGCTTCTGAAAGTGTGGTGCGGTGCCTGTCTACGCCCACTGGCTGGACTCA
GTCTCCTACCATCCCCCTCCTGGTATCTAGGCTCAAGGCCCTCCAGTGCCCCCTGCCCTGACACTTTC
TGCTCTGACCCTCTCTTTGGATTCATGTGCCGTCCTATGGCTTCACATGGGGCTTTTCGCCCCCAGGC
CTCTGGTCTACATCTCTACAAAGTGCTAAGAGCTTGTCCATCCCAGAGGAGGCTGAACGAGTGTCTGC
ACAACAATGGCGGCTGCTCACACATCTGCACTGACCTCAAGATTGGCTTTGAATGCACGTGCCCAGCA
GGCTTCCAGCTCCTGGACCAGAAGACCTGTGGCGACATTGATGAGTGCAAGGACCCAGATGCCTGCAG
CCAGATCTGTGTCAATTACAAGGGCTATTTTAAGTGTGAGTGCTACCCTGGCAACGAGATGGACCTAC
TGACCAAGAACTGCCAAGCTGCTGCTGGAAAGAGCCCATCCCTAATCTTCACCAACCGGTACGAGGTG
CGGAGGATCGACCTGGTGAAGCGGAACTATTCACGCCTCATCCCCATGCTCAAGAATGTCGTGGCACT
AGATGTGGAAGTTGCCACCAATCGCATCTACTGGTGTGACCTCTCCTACCGTAAGATCTATAGCGCCT
ACATGGACAAGGCCAGTGACCCGAAAGAGCAGGAGGTCCTCATTGACGAGCAGTTGCACTCTCCAGAG
GGCCTGGCAGTGGACTGGGTCCACAAGCACATGTACTGGACTGACTCGGGCAATAAGACCATCTCAGT
GGCCACAGTTGATGGTGGCCGCCGACGCACTCTCTTCAGCCGTAACCTCAGTGAACCCCGGGCCATCG
CTGTTGACCCCCTGCGAGGGTTCATGTATTGGTCTGACTGGGGGGACCAGGCCAAGATTGAGAAATCT
GGGCTCAACGGTGTGGACCGGCAAACACTGGTGTCAGACAATATTGAATGGCCCAACGGAATCACCCT
GGATCTGCTGAGCCAGCGCTTGTACTGGGTAGACTCCAAGCTACACCAACTGTCCAGCATTGACTTCA
GTGGAGGCAACAGAAAGACGCTGATCTCCTCCACTGACTTCCTGAGCCACCCTTTTGGGATAGCTGTG
TTTGAGGACAAGGTGTTCTGGACAGACCTGGAGAACGAGGCCATTTTCAGTGCAAATCGGCTCAATGG
CCTGGAAATCTCCATCCTGGCTGAGAACCTCAACAACCCACATGACATTGTCATCTTCCATGAGCTGA
AGCAGCCAAGAGCTGGAGATGCCTGTGAGCTGAGTGTCCAGCCTAATGGAGGCTGTGAATACCTGTGC
CTTCCTGCTCCTCAGATCTCCAGCCACTCTCCCAAGTACACATGTGCCTGTCCTGACACAATGTGGCT
GGGTCCAGACATGAAGAGGTGCTACCGAGCACCTCAATCTACCTCAACTACGACGTTAGCTTCTACCA
TGACGAGGACAGTACCTGCCACCACAAGAGCCCCCGGGACCACCGTCCACAGATGGACCTACCAGAAC
CACAGCACAGAGACACCAAGCCTGACAGCTGCAGTCCCAAGCTCAGTTAGTGTCCCCAGGGCTCCCAG
CATCAGCCCGTCTACCCTAAGCCCTGCAACCAGCAACCACTCCCAGCACTATGCAAATGAGAACAGTA
AGATGGGCTCAACAGTCACTGCCGCTGTTATCGGGATCATCGTGCCCATAGTGGTGATAGCCCTCCTG
TGCATGAGTGGATACCTGATCTGGAGAAACTGGAAGCGGAAGAACACCAAAAGCATGAATTTTGACAA
CCCAGTCTACAGGAAAACAACAGAAGAAGAAGATGAAGATGAGCTCCATATAGGGAGAACTGCTCAGA
TTGGCCATGTCTATCCTGCAGCAATGAGCAGCTTTGATCGCCCACTGTGGGCAGAGCCCTGTCTTGGG
GAGACCAGAGAACCGGAAGACCCAGCCCCTGCCCTCAAGGAGCTTTTTGTCTTGCCGGGGGAACCAAG
GTCACAGCTGCACCAACTCCCGAAGAACCCTCTTTCCGAGCTGCCTGTCGTCAAATCCAAGCAAGTGG
CATTAAGCCTTGAAGATGATGGACTACCCTGAGGATGGGATCACCCCCTTCGTGCCTCATGGAATTCA
GTCCCATGCACTACACTCTGGATGGTGTATGACTGGATGAATGGGTTTCTATATATGGGTCTGTGTGA
GTGTATGTGTGTGTGTGATTTTTTTTTTAAATTTATGTTGCGGAAAGGTAACCACAAAGTTATGATGA
ACTGCAAACATCCAAAGGATGTGAGAGTTTTTCTATGTATAATGTTTTATACACTTTTTAACTGGTTG
CACTACCCATGAGGAATTCGTGGAATGGCTACTGCTGACTAACATGATGCACATAACCAAATGGGGGC
CAATGGCACAGTACCTTACTCATCATTTAAAAACTATATTTACAGAGATGTTTGGTTGCTGGGGGGGG
CTTTTTTGGGTTTTGGGGCATTTGTTTTTTGTAAATAAGATGATTATGCTTTGTGGCTATCCATCAAC
ATAAGTAAAAAAAAAAAAAAAACACTTCAACTCCCTCCCCCATTTAGATTATTTATTAACATATTTTA
AAAATCAGATGAGTTCTATAAATAATTTAGAGAAGTGAGAGTATTTATTTTTGGCATGTTTGGCCCAC
CACACAGACTCTGTGTGTGTATGTGTGTGTTTATATGTGTATGTGTGTGACAGGAAAATCTGTAGAGA
AGAGGCACATCTATGGCTACTGTTCAAATACATAAAGATAAATTTATTTTCACACAGTCCACAAGGGG
TATATCTTGTAGTTTTCAGAAAAGCCTTTGGAAATCTGGATCAGGAAATAGATACCATGGTTTGTGCA
ATTATGTAGTAAAAAAGGCAAATCTTTTCACCTCTGGCTATTCCTGAGACCCCAGGAAGTCAGGAAAA
GCCTTTCAGCTCACCCATGGCTGCTGTGACTCCTACCAGGGCTTTCTTGGCTTTGGCGAAGGTCAGTG
TACAGACATTCCATGGTACCAGAGTGCTCAGAAAGTCAAGATAGGATATGCCTCACCCTCAGCTACTC
CTTGTTTTAAAGTTCAGCTCTTTGAGTAACTTCTTCAATTTCTTTCAGGACACTTGGGTTGAATTCAG
TAAGTTTCCTCTGAAGCACCCTGAAGGGTGCCATCCTTACAGAGCTAAGTGGAGACGTTTCCAGATCA
GCCCAAGTTTACTATAGAGACTGGCCCAGGCACTGATGTCTAGGACATGCTGTGGATGAAGGATAAAG
ATGGTGGAATAGGTTTTATCACATCTCTTATTTCTCTTTTCCCCTTACTCTCTACCATTTCCTTTATG
TGGGGAAACATTTTAAGGTAATAAATAGGTTACTTACCATCATATGTTCATATAGATGAAACTAATTT
TTGGCTTAAGTCAGAACAACTGGCCCCCAATTGAAGTCATATTTGTGGGGGGAAATGGCATACGCAAT
ATTATATTATATTGGATATTTATGTTCACACAGGAATTTGGTTTACTGCTTTGTAAATAAAAGGGAAA
ACTCCGGGTA

| | | | |
|---|---|---|---|
| NOV28n, SNP13382498 of | SEQ ID NO:664 | 905 aa | MW at 100433.3 kD |
| CG55256-07, | SNP Pos: 894 | SNP Change: Arg to Gln | |
| Protein Sequence | | | |

MGLPEPGPLRLLALLLLLLLLLLLRLQHLAAAAADPLLGGQGPAKECEKDQFQCRNERCIPSVWRCDE
DDDCLDHSDEDDCPKKTCADSDFTCDNGHCIHERWKCDGEEECPDGSDESEATCTLGTCRGDEFQCGD
GTCVLAIKHCNQEQDCPDGSDEAGCLQESPCEGPRRFQCKSGKRVDGGKVCDVQRDCRDWSDELLKVW
CGACLRPLAGLSLLPSPSWYLGSRPSSAPCPDTFCSDPLFGFMCRPMASHGAFRPQASGLHLYKVLRA
CPSQRRLNECLHNNGGCSHICTDLKIGFECTCPAGFQLLDQKTCGDIDECKDPDACSQICVNYKGYFK
CECYPGYEMDLLTKNCQAAAGKSPSLIFTNRYEVRRIDLVKRNYSRLIPMLKNVVALDVEVATNRIYW
CDLSYRKIYSAYMDKASDPKEQEVLIDEQLHSPEGLAVDWVHKHIYWTDSGNKTISVATVDGGRRRTL
FSRNLSEPAAIAVDPLRGFMYWSDWGDQAKIEKSGLNGVDRQTLVSDNIEWPNGITLDLLSQRLYWVD
SKLHQLSSIDFSGGNRKTLISSTDFLSHPFGIAVFEDKVFWTDLENEAIFSANRLNGLEISILAENLN
NPHDIVIFHELKQPRAPDACELSVQPNGGCEYLCLPAPQISSHSPKYTCACPDTMWLGPDMKRCYRAP
QSTSTTTLASTMTRTVPATTRAPGTTVHRSTYQNHSTETPSLTAAVPSSVSVPRAPSISPSTLSPATS
NHSQHYANEDSKMGSTVTAAVIGIIVPIVVIALLCMSGYLIWRNWKRKNTKSMNFDNPVYRKTTEEED
EDELHIGRTAQIGHVYPAAISSFDRPLWAEPCLGETREPEDPAPALKELFVLPGEPRSQLHQLPKNPL
SELPVVKSKQVALSLEDDGLP

A ClustalW comparison of the above protein sequences yields the following sequence alignment shown in Table 28B.

TABLE 28B
Comparison of the NOV28 protein sequences.

| | | |
|---|---|---|
| NOV28a | ----------MGLPEPGPLRLLALLLLLLLLLLLRLQHLAAAAADPLLGGQGPAKECEKD | (SEQ ID NO:638) |
| NOV28b | ------------------------------------------AADPLLGGQGPAKDCEKD | (SEQ ID NO:640) |
| NOV28c | ----------------------------------------GSAADPLLGGQGPAKDCEKD | (SEQ ID NO:642) |
| NOV28d | MPLSWPVSFPQRSLLPDPLELLCVLFVSFFLLLLQLQHLAAAAADPLLGGQGPAKDCEKD | (SEQ ID NO:644) |
| NOV28e | ------MGLPE----PGPLRLLALLLLLLLLLLLRLQHLAAAAADPLLGGQGPAKECEKD | (SEQ ID NO:646) |
| NOV28f | ------MGLPE----PGPLRLLALLLLLLLLLLLRLQHLAAAAADPLLGGQGPAKDCEKD | (SEQ ID NO:648) |
| NOV28g | MPLSWPVSFPQRSLLPDPLELLCVLFVSFFLLLLQLQHLAAAAADPLLGGQGPAKDCEKD | (SEQ ID NO:650) |
| NOV28h | ----------MGLPEPGPLRLLALLLLLLLLLLLRLQHLAAAAADPLLGGQGPAKECEKD | (SEQ ID NO:652) |
| NOV28i | ------MGLPE----PGPLRLLALLLLLLLLLLLQLQHLAAAAADPLLGGQGPAKDCEKD | (SEQ ID NO:654) |
| NOV28 | ------MGLPE----PGPLRLLALLLLLLLLLLLRLQHLAAAAADPLLGGQGPAKECEKD | (SEQ ID NO:656) |
| NOV28k | ------MGLPE----PGPLRLLALLLLLLLLLLLRLQHLAAAAADPLLGGQGPAKDCEKD | (SEQ ID NO:658) |
| NOV281 | ------MGLPE----PGPLRLLALLLLLLLLLLLRLQHLAAAAADPLLGGQGPAKECEKD | (SEQ ID NO:660) |
| NOV28a | QFQCRNERCIPSVWRCDEDDDCLDHSDEDDCP--KKTCADSDFTCDNGHCIHERWKCDGE | |
| NOV28b | QFQCRNERCIPSVWRCDEDDDCLDHSDEDDCP--KKTCADSDFTCDNGHCIHERWKCDGE | |
| NOV28c | QFQCRNERCIPSVWRCDEDDDCLDHSDEDDCP--KKTCADSDFTCDNGHCIHERWKCDGE | |
| NOV28d | QFQCRNERCIPSVWRCDEDDDCLDHSDEDDCPAAKKTPVRSDFTCDNGHCIHERWKCDGE | |
| NOV28e | QFQCRNERCIPSVWRCDEDDDCLDHSDEDDCP--KKTCVRSDFTCDNGHCIHERWKCDGE | |
| NOV28f | QFQCRNERCIPSVWRCDEDDDCLDHSDEDDCP--KKTCVRSDFTCDNGHCIHERWKCDGE | |
| NOV28g | QFQCRNERCIPSVWRCDEDDDCLDHSDEDDCPAAKKTPVRSDFTCDNGHCIHERWKCDGE | |
| NOV28h | QFQCRNERCIPSVWRCDEDDDCLDHSDEDDCP--KKTCVRSDFTCDNGHCIHERWKCDGE | |
| NOV28i | QFQCRNERCIPSVWRCDEDDDCLDHSDEDDCP--KKTCADSDFTCDNGHCIHERWKCDGE | |
| NOV28 | QFQCRNERCIPSVWRCDEDDDCLDHSDEDDCP--KKTCADSDFTCDNGHCIHERWKCDGE | |
| NOV28k | QFQCRNERCIPSVWRCDEDDDCLDHSDEDDCP--KKTCVRSDFTCDNGHCIHERWKCDGE | |
| NOV28l | QFQCRNERCIPSVWRCDEDDDCLDHSDEDDCP--KKTCADSDFTCDNGHCIHERWKCDGE | |
| NOV28a | EECPDGSDESEATCTLGTCRGDEFQC--GDGTCVLAIKHCNQEQDCPDGSDEAGCLQE-- | |
| NOV28b | EECPDGSDESEATCTKQVC----------------------------------------- | |
| NOV28c | EECPDGSDESEATCTKQVCPAEKLSCGPTSHKCVPASWRCDGEKDCEGGADEAGCATS-- | |
| NOV28d | EECPDGSDESEATCTKQVCPAEKLSCGPTSHKCVPASWRCDGEKDCEGGADEAGCATS-- | |
| NOV28e | EECPDGSDESEATCTKQVCPAEKLSCGPTSHKCVPASWRCDGEKDCEGGADEAGCATLCA | |
| NOV28f | EECPDGSDESEATCTKQVCPAEKLSCGPTSHKCVPASWRCDGEKDCEGGADEAGCATS-- | |
| NOV28g | EECPDGSDESEATCTKQVCPAEKLSCGPTSHKCVPASWRCDGEKDCEGGADEAGCATS-- | |
| NOV28h | EECPDGSDESEATCTLGTCRGDEFQC--GDGTCVLAIKHCNQEQDCPDGSDEAGCLQE-- | |
| NOV28l | EECPDGSDESEATCTKQVCPAEKLSCGPTSHKCVPASWRCDGEKDCEGGADEAGCATS-- | |
| NOV28 | EECPDGSDESEATCTKQVCPAEKLSCGPTSHKCVPASWRCDGEKDCEGGADEAGCATS-- | |
| NOV28k | EECPDGSDESEATCTKQVCPAEKLSCGPTSHKCVPASWRCDGEKDCEGGADEAGCATS-- | |
| NOV28l | EECPDGSDESEATCTKQVCPAEKLSCGPTSHKCVPASWRCDGEKDCEGGADEAGCATLCA | |
| NOV28a | ------------------------------------------------------------ | |
| NOV28b | ------------------------------------------------------------ | |
| NOV28c | ------------------------------------------------------------ | |
| NOV28d | ------------------------------------------------------------ | |
| NOV28e | PHEFQCGNRSCLAAVFVCDGDDDCGDGSDERGCADPACGPREFRCGGDGGGACIPERWVC | |
| NOV28f | ------------------------------------------------------------ | |
| NOV28g | ------------------------------------------------------------ | |
| NOV28h | ------------------------------------------------------------ | |
| NOV28i | ------------------------------------------------------------ | |
| NOV28 | ------------------------------------------------------------ | |
| NOV28k | ------------------------------------------------------------ | |
| NOV28l | PHEFQCGNRSCLAAVFVCDGDDDCGDGSDERGCADPACGPREFRCGGDGGGACIPERWVC | |
| NOV28a | ------------------------------------------------------------ | |
| NOV28b | ------------------------------------------------------------ | |
| NOV28c | ------------------------------------------------------------ | |
| NOV28d | ------------------------------------------------------------ | |
| NOV28e | DRQFDCEDRSDEAAELCGRPGPGATSAPAACATVSQFACRSGECVHLGWRCDGDRDCKDK | |
| NOV28f | ------------------------------------------------------------ | |
| NOV28g | ------------------------------------------------------------ | |
| NOV28h | ------------------------------------------------------------ | |
| NOV28i | ------------------------------------------------------------ | |
| NOV28 | ------------------------------------------------------------ | |
| NOV28k | ------------------------------------------------------------ | |
| NOV28l | DRQFDCEDRSDEAAELCGRPGPGATSAPAACATVSQFACRSGECVHLGWRCDGDRDCKDK | |
| NOV28a | -------SPCEGPRRFQCKSGKRVDGGKVCDVQRDCRDWSDELLKVWCGACLRPLAGLSL | |
| NOV28c | -------LGTCRGDEFQCGDGTCVLAIKHCNQEQDCPDGSDEAGCLQG------------ | |
| NOV28d | -------LGTCRGDEFQCGDGTCVLAIKHCNQEQDCPDGSDEAGCLQG------------ | |
| NOV28e | SDEADCPLGTCRGDEFQCGDGTCVLAIKHCNQEQDCPDGSDEAGCLQESPCEGPRRFQCK | |
| NOV28f | -------LGTCRGDEFQCGDGTCVLAIKHCNQEQDCPDGSDEAGCLQG------------ | |
| NOV28g | -------LGTCRGDEFQCGDGTCVLAIKHCNQEQDCPDGSDEAGCLQG------------ | |
| NOV28h | -------SPCEGPRRFQCKSGKRVDGGKVCDVQRDCRDWSDELLKVWCGACLRPLAGLSL | |
| NOV28i | -------LGTCRGDEFQCGDGTCVLAIKHCNQEQDCPDGSDEAGCLQG------------ | |
| NOV28 | -------LGTCRGDEFQCGDGTCVLAIKHCNQEQDCPDGSDEAGCLQG------------ | |
| NOV28k | -------LGTCRGDEFQCGDGTCVLAIKHCNQEQDCPDGSDEAGCLQG------------ | |
| NOV28a | LPSPSWYLGSRPSSAPCPDTFCSDPLFGFMCRPMASHGAFRPQASGLHLYKVLRACPSQR | |
| NOV28b | ------------------------------------------------------------ | |
| NOV28c | ------------------------------------------------------------ | |
| NOV28d | ------------------------------------------------------------ | |
| NOV28e | SGKRVDGGKVCDVQRDCRDWSDELLKVWCVSYHPPPG----------------------- | |
| NOV28f | ------------------------------------------------------------ | |
| NOV28g | ------------------------------------------------------------ | |
| NOV28h | LPSPSWSPCP----SP-------------------G------------------------ | |
| NOV28i | G----------------------------------------------------------- | |
| NOV28 | ------------------------------------------------------------ | |
| NOV28k | ------------------------------------------------------------ | |
| NOV28l | ------------------------------------------------------------ | |
| NOV28a | RLNECLHNNGGCSHICTDLKIGFECTCPAGFQLLDQKTCGDIDECKDPDACSQICVNYKG | |
| NOV28b | ------------------------------------------------------------ | |
| NOV28c | -LNECLHNNGGCSHICADLKIGFECTCPAGFQLLDQKTCGDIDECKDPDACSQICVNYKG | |
| NOV28d | -LNECLHNNGGCSHICTDLKIGFECTCPAGFQLLDQKTCG-------------------- | |
| NOV28e | -LNECLHNNGGCSHICTDLKIGFECTCPAGFQLLDQKTCGDIDECKDPDACSQICVNYKG | |
| NOV28f | -LNECLHNNGGCSHICTDLKIGFECTCPAGFQLLDQKTCGDIDECKDPDACSQICVNYKG | |
| NOV28g | -LNECLHNNGGCSHICTDLKIGFECTCPAGFQLLDQKTCG-------------------- | |
| NOV28h | -LNECLHNNGGCSHICTDLKIGFECTCPAGFQLLDQKTCGDIDECKDPDACSQICVNYKG | |
| NOV28i | -LNECLHNNGGCSHICTDLKIGFECTCPAGFQLLDQKTCGDIDECKDPDACSQTCVNYKG | |
| NOV28 | -LNECLHNNGGCSHICTDLKIGFECTCPAGFQLLDQKTCGDIDECKDPDACSQICVNYKG | |
| NOV28k | -LNECLHNNGGCSHICTDLKIGFECTCPAGFQLLDQKTCGDIDECKDPDACSQICVNYKG | |
| NOV28l | -LNECLHNNGGCSHICTDLKIGFECTCPAGFQLLDQKTCGDIDECKDPDACSQICVNYKG | |
| NOV28a | YFKCECYPGYEMDLLTKNCKAAAGKSPSLIFTNRYEVRRIDLVKRNYSRLIPMLKNVVAL | |
| NOV28b | ------------------------------------------------------------ | |
| NOV28c | YFKCECYPGYEMDLLTKNCKAAAGKSPSLIFTNRYEVRRIDLVKRNYSRLIPMLKNVVAL | |
| NOV28d | ----------------------AGKSPSLIFTNRHEVRRIDLVKRNYSRLIPMLKNVVAL | |
| NOV28e | YFKCECYPGYEMDLLTKNCKAAAGKSPSLIFTNRYEVRRIDLVKRNYSRLIPMLKNVVAL | |
| NOV28f | YFKCECYPGYEMDLLTKNCKAAAGKSPSLIFTNRYEVRRIDLVKRNYSRLIPMLKNVVAL | |
| NOV28g | ----------------------AGKSPSLIFTNRYEVRRIDLVKRNYSRLIPMLKNVVAL | |
| NOV28h | YFKCECYPGYEMDLLTKNCKAAAGKSPSLIFTNRYEVRRIDLVKRNYSRLIPMLKNVVAL | |
| NOV28i | YFKCECYPGYEMDLLTKNCKAAAGKSPSLIFTNRYEVRRIDLVKRNYSRLIPMLKNVVAL | |
| NOV28 | YFKCECYPGYEMDLLTKNCKAAAGKSPSLIFTNRYEVRRIDLVKRNYSRLIPMLKNVVAL | |
| NOV28k | YFKCECYPGYEMDLLTKNCKAAAGKSPSLIFTNRYEVRRIDLVKRNYSRLIPMLKNVVAL | |
| NOV28l | YFKCECYPGYEMDLLTKNCKAAAGKSPSLIFTNRYEVRRIDLVKRNYSRLIPMLKNVVAL | |
| NOV28a | DVEVATNRIYWCDLSYRKIYSAYMDKASDPKEQEVLIDEQLHSPEGLAVDWVHKHIYWTD | |
| NOV28b | -----------------------------------------------------P--AEK- | |
| NOV28c | DVEVATNRIYWCDLSYRKIYSAYMDKASDPKEQEVLIDEQLHSPEGLAVDWVHKHIYWTD | |
| NOV28d | DVEVATNRIYWCDLSYRKIYSAYMDKASDPKEQEVLIDEQLHSPEGLAVDWVHKHIYWTD | |
| NOV28e | DVEVATNRIYWCDLSYRKIYSAYMDKASDPKEQEVLIDEQLHSPEGLAVDWVHKHIYWTD | |
| NOV28f | DVEVATNRIYWCDLSYRKIYSAYMDKASDPKEQEVLIDEQLHSPEGLAVDWVHKHIYWTD | |
| NOV28g | DVEVATNRIYWCDLSYRKIYSAYMDKASDPKEQEVLIDEQLHSPEGLAVDWVHKHIYWTD | |
| NOV28h | DVEVATNRIYWCDLSYRKIYSAYMDKASDPKEQEVLIDEQLHSPEGLAVDWVHKHIYWTD | |
| NOV28i | DVEVATNRIYWCDLSYRKIYSAYMDKASDPKEQEVLIDEQLHSPEGLAVDWVHKHIYWTD | |
| NOV28 | DVEVATNRIYWCDLSYRKIYSAYMDKASDPKEQEVLIDEQLHSPEGLAVDWVHKHIYWTD | |
| NOV28k | DVEVATNRIYWCDLSYRKIYSAYMDKASDPKEQEVLIDEQLHSPEGLAVDWVHKHIYWTD | |
| NOV28l | DVEVATNRIYWCDLSYRKIYSAYMDKASDPKEQEVLIDEQLHSPEGLAVDWVHKHIYWTD | |
| NOV28a | SGNKTISVATVDGGRRRTLFSRNLSEPRAIAVDPLRGFMYWSDWGDQAKIEKSGLNGVDR | |
| NOV28b | ------------------------------------------------------------ | |
| NOV28c | SGNKTISVATVDGGRRRTLFSRNLSEPRAIAVDPLRGFMYWSDWGDQAKIEKSGLNGVDR | |
| NOV28d | SGNKTISVATVDGGRRRTLFSRNLSEPRAIAVDPLRGFMYWSDWGDQAKIEKSGLNGVDR | |
| NOV28e | SGNKTISVATVDGGRPRTLFSRNLSEPBAIAVDPLRGFMYWSDWGDQAKIEKSGLNGVDR | |
| NOV28f | SGN-----KNISGGRRRTLFSRNLSEPPAIAVDPLRGFMYWSDWGDQAKIEKSGLNGVDR | |
| NOV28g | SGNKTISVATVDGGRRRTLFSRNLSEPRAIAVDPLRGFMYWSDWGDQAKIEKSGLNGvDR | |
| NOV28h | SGNKTISVATVDGGRRRTLFSRNLSEPRAIAVDPLRGFMYWSDWGDQAKIEKSGLNGVDR | |
| NOV28i | SGNKTISVATVDGGRRRTLFSRNLSEPRAIAVDPLRGFMYWSDWGDQAKIEKSGLNGVDR | |
| NOV28 | SGNKTISVATVDGGRPRTLFSRNLSEPRAIAVGPLRGFMYWSDWGDQAKIEKSGLNGVDR | |
| NOV28k | SGNKTISVATVDGGRRRTLFSRNLSEPRAIAVDPLRGFMYWSDWGDQAKIEKSGLNGVDR | |
| NOV28l | SGNKTISVATVDGGRRRTLFSRNLSEPRAIAVDPLRGFMYWSDWGDQAKIEKSGLNGVDR | |
| NOV28a | QTLVSDNIEWPNGITLDLLSQRLYWVDSKLHQLSSIDFSGGNRKTLISSTDFLSHPFGIA | |
| NOV28b | ------------------------------------------------------------ | |
| NOV28c | QTLVSDNIEWPNGITLDLLSQRLYWVDSKLHQLSSIDFSGGNRKTLISSTDFLSHPFGIA | |
| NOV28d | QTLVSDNIEWPNGITLDLLSQRLYWVDSKLHQLSSIDFSGGNRKTLISSTDFLSHPFGIA | |
| NOV28e | QTLVSDNIEWPNGITLDLLSQRLYWVDSKLHQLSSIDFSGGNRKTLISSTDFLSHPFGIA | |
| NOV28f | QTLVSDNIEWPNGITLDLLSQRLYWVDSKLHQLSSIDFSGGNRKTLISSTDFLSHPFGIA | |
| NOV28g | QTLVSDNIEWPNGITLDLLSQRLYWVDSKLHQLSSIDFSGGNRKTLISSTDFLSHPFGIA | |
| NOV28h | QTLVSDNIEWPNGITLDLLSQRLYWVDSKLHQLSSIDFSGGNRKTLISSTDFLSHPFGIA | |
| NOV28i | QTLVSDNIEWPNGITLDLLSQRLYWVDSKLHQLSSIDFSGGNRKTLISSTDFLSHPFGIA | |
| NOV28 | QTLVSDNIEWPNGITLDLLSQRLYWVDSKLHQLSSIDFSGGNRKTLISSTDFLSHPFGIA | |
| NOV28k | QTLVSDNIEWPNGITLDLLSQRLYWVDSKLHQLSSIDFSGGNRKTLISSTDFLSHPFGIA | |
| NOV28l | QTLVSDNIEWPNGITLDLLSQRLYWVDSKLHQLSSIDFSGGNRKTLISSTDFLSHPFGIA | |
| NOV28a | VFEDKVFWTDLENEAIFSANRLNGLEISILAENLNNPHDIVIFHELKQPRAPDACELSVQ | |
| NOV28b | ------------------------------------------------------------ | |
| NOV28c | VFEDKVFWTDLENEAIFSANRLNGLEISILAENLNNPHDIVIFHELKQPRAPDACELSVQ | |
| NOV28d | VFEDKVFWTDLENEAIFSANRLNGLEISILAENLNNPHDIVIFHELKQPEAPDACELSVQ | |
| NOV28e | VFEDKVFWTDLENEAIFSANRLNGLEISILAENLNNPHDIVIFHELKQPRAPDACELSVQ | |
| NOV28f | VFEDKVFWTDLENEAIFSANRLNGLEISILAENLNNPHDIVIFHELKQPRAPDACELSVQ | |
| NOV28g | VFEDKVFWTDLENEAIFSANRLNGLEISILAENLNNPHDIVIFHELKQPPAPDACELSVQ | |
| NOV28h | VFEDKVFWTDLENEAIFSANRLNGLEISILAENLNNPHDIVIFHELKQPRAPDACELSVQ | |
| NOV28i | VFEDKVFWTDLENEAIFSANRLNGLEISILAENLNNPHDIVIFHELKQPRAPDACELSVQ | |
| NOV28 | VFEDKVFWTDLENEAIFSANRLNGLEISILAENLNNPHDIVIFHELKQPRAPDACELSVQ | |
| NOV28k | VFEDKVFWTDLENEAIFSANRLNGLEISILAENLNNPHDIVIFHELKQPRAPDACELSVQ | |
| NOV28l | VFEDKVFWTDLENEAIFSANRLNGLEISILAENLNNPHDIVIFHELKQPRAPDACELSVQ | |
| NOV28a | PNGGCEYLCLPAPQISSHSPKYTCACPDTMWLGPDMKRCYRAP----------------- | |
| NOV28b | -------------------------------LGPDMKRCYFAP----------------- | |
| NOV28c | PNGGCEYLCLPAPQISSHSPKYTCACPDTMWLGPDMKRCYRAP----------------- | |
| NOV28d | PNGGCEYLCLPAPQISSHSPKYTCACPDTMWLGPDMKRCYEAP----------------- | |
| NOV28e | PNGGCEYLCLPAPQISSHSPKYTCACPDTMWLGPDMKRCYEAPQSTSTTTLASTMTRTVP | |
| NOV28f | PNGGCEYLCLPAPQISSHSPKYTCACPDTMWLGPDMKRCYRD------------------ | |
| NOV28g | PNGGCEYLCLPAPQISSHSPKYTCACPDTMWLGPDMKRCYRAP----------------- | |
| NOV28h | PNGGCEYLCLPAPQISSHSPKYTCACPDTMWLGPDMKRCYRAP----------------- | |
| NOV28i | PNGGCEYLCLPAPQISSHSPKYTCACPDTMWLGPDMKRCYRAP----------------- | |
| NOV28 | PNGGCEYLCLPAPQISSHSPKYTCACPDTMWLGPDMKRCYRAPQSTSTTTLASTMTRTVP | |
| NOV28k | PNGGCEYLCLPAPQISSHSPKYTCACPDTMWLGPDMKRCYRAP----------------- | |
| NOV28l | PNGGCEYLCLPAPQISSHSPKYTCACPDTMWLGPDMKRCYRAPQSTSTTTLASTMTRTVP | |
| NOV28a | ------------------------------------------------------------ | |
| NOV28b | ------------------------------------------------------------ | |
| NOV28c | ------------------------------------------------------------ | |
| NOV28d | ------------------------------------------------------------ | |
| NOV28e | ATTRAPGTTVHRSTYQNHSTETPSLTAAVPSSVSVPRAPSISPSTLSPATSNHSQHYANE | |
| NOV28f | ---------------------------------------------------------ANE | |
| NOV28g | ------------------------------------------------------------ | |
| NOV28h | ------------------------------------------------------------ | |
| NOV28i | ------------------------------------------------------------ | |
| NOV28 | ATTRAPGTTVHRSTYQNHSTETPSLTAAVPSSVSVPRAPSISPSTLSPATSNHSQHYANE | |
| NOV28k | ------------------------------------------------------------ | |
| NOV28l | ATTRAPGTTVHRSTYQNHSTETPSLTAAVPSSVSVPRAPSISPSTLSPATSNHSQHYANE | |
| NOV28a | -QSTSTTTLASTMTRTVPATTRAPGTTVHRSTYQNHSTETPSLTAAVPSSVSVPRAPSIS | |
| NOV28b | -QSTSTTTLASTMTRTVPATTRAPGTTVHRSTYQNHSTETPSLTAAVPSSVSVPPAPSIS | |
| NOV28c | -QSTSTTTLASTMTRTVPATTRAPGTTVHRSTYQNHSTETPSLTAAVPSSVSVPRVPSIS | |
| NOV28d | -QSTSTTTLASTMTRTVPATTRAPGTTVHRSTYQNHSTETPSLTAAVPSSVSVPRAPSIS | |
| NOV28e | DSKMGSTVTAAVIGIIVPIVVIALLCMSGYLIWRNWKRKN-TKSMMFDNPVYRKTTEEED | |
| NOV28f | DSKMGSTVTAAVIGIIVPIVVIALLCMSGYLIWRNWKRKN-TKSMMFDNPVYRKTTEEED | |
| NOV28g | -QSTSTTTLASTMTRTVPATTRAPGTTVHRSTYQNHSTETPSLTAAVPSSVSVPRAPSIS | |
| NOV28h | -QSTSTTTLASTMTRTVPATTRAPGTTVHRSTYQNHSTETPSLTAAVPSSVSVPRAPSIS | |
| NOV28i | -QSTSTTTLASTMTRTVPATTRAPGTTVHRSTYQNHSTETPSLTAAVPSSVSVPRAPSIS | |
| NOV28 | DSKMGSTVTAAVIGIIVPIVVIALLCMSGYLIWRNWKRKN-TKSMMFDNPVYRKTTEEED | |
| NOV28k | -QSTSTTTLASTMTRTVPATTRAPGTTVHRSTYQNHSTETPSLTAAVPSSVSVPRAPSIS | |
| NOV28l | DSKMGSTVTAAVIGIIVPIVVIALLCMSGYLIWRNWKRKN-TKSMMFDNPVYRKTTEEED | |
| NOV28a | PSTLSPATSNHSQHYANEDSKNGSTVTAAVIGIIVPIVVIALLCMSGYLIWRNWK----- | |
| NOV28b | PSTLSPATSNHSQHYANEDSKMGSTVTAAVIGIIVPIVVTALLCMSGYLIWRNWK----- | |
| NOV28c | P----------------------------------------------------------- | |
| NOV28d | PSTLSPATSNHSQHAN-EDSKMGSTVTAAVIGIIVPIANEDSKMGSTVTAAVIGIIVPIG | |
| NOV28e | E----------------------------------------------------------- | |
| NOV28f | E----------------------------------------------------------- | |
| NOV28g | PSTLSPATSNHSQHYANEDSKMGSTVTAAVIGIIVPIV-------------VIALLCMSG | |
| NOV28h | PSTLSPATSNHSQHYANEDSKMGSTVTAAVIGIIVPIVVIALLCMSGYLIWRNWK----- | |
| NOV28i | P----------------------------------------------------------- | |
| NOV28 | E----------------------------------------------------------- | |
| NOV28k | P----------------------------------------------------------- | |
| NOV28l | E----------------------------------------------------------- | |
| NOV28a | ----------------RKNTKSMNFDNPVYRKTTEEEDEDELHIGRTAQIGHVYPAAISS | |
| NOV28b | ----------------RKNTKSMNFDNPVYRKTTEEEDEDELHIGRTAQIGHVYPAAISS | |
| NOV28c | ---------------------------------------STLRPATSNHSQHYAN----- | |
| NOV28d | DSPPVHEWIPDLEKLEAEEHQKHEFDNPVYRKTTEEEDEDELHIGRTAQIGHVYPAAISS | |
| NOV28e | ---------------------------------------DELHIGRTAQIGHVYPAAISS | |
| NOV28f | ---------------------------------------DELHIGRTAQIGHVYP----- | |
| NOV28g | -------YLIWRNWKRKNTKSMNFDNPVYRICTTEEEDEDELHIGRTAQIGHVYPAAISS | |
| NOV28h | ---------------RKNTKSMNFDNPVYRICTTEEEDEDELHIGRTAQIGHVYPAAISS | |
| NOV28i | ---------------------------------------STLSPATSNHSQHYAN----- | |
| NOV28 | ---------------------------------------DELHIGRTAQIGHVYP----- | |
| NOV28k | ---------------------------------------STLSPATSNHSQHYAN----- | |
| NOV28l | ---------------------------------------DELHIGRTAQIGHVYPAAISS | |
| NOV28a | FDRPLWAEPCLGETREPEDPAPALKELFVLPGEPRSQLHQLPKNPLSELPVVKSK-RVAL | |
| NOV28b | FDRPLWAEPCLGETREPEDPAPALKELFVLPGEPRSQLHQLPKNPLSELPVVKSK-RVAL | |
| NOV28c | ------------------------------------------------------------ | |
| NOV28d | FDRPLWAEPCLGETREPEDPAPALKELFVLPGEPRSQLHQLPKNPLSELPVVKSKQRVAL | |
| NOV28e | FDRPLWAEPCLGETREPEDPAPALKELFVLPGEPRSQLHQLPKNPLSELPVVKSK-RVAL | |
| NOV28f | ------------------------------------------------------A-RVAL | |
| NOV28g | FDRPLWAEPCLGETREPEDPAPALKELFVLPGEPRSQLHQLPKNPLSELPVVKSK-RVAL | |
| NOV28h | FDRPLWAEPCLGETREPEDPAPALKELFVLPGEPRSQLHQLPKNPLSELPVVKSK-RVAL | |
| NOV28i | ------------------------------------------------------------ | |
| NOV28 | ------------------------------------------------------A-RVAL | |
| NOV28k | ------------------------------------------------------------ | |
| NOV28l | FDRPLWAEPCLGETREPEDPAPALKELFVLPGEPRSQLHQLPKNPLSELPVVKSK-RVAL | |
| NOV28a | SLEDDGLP---------------------------------------------------- | |
| NOV28b | SLEDDGLP---------------------------------------------------- | |
| NOV28c | --EDSKLE---------------------------------------------------- | |
| NOV28d | SLEDDGLP---------------------------------------------------- | |
| NOV28e | SLEDDGLP---------------------------------------------------- | |
| NOV28f | SLEDDGLP---------------------------------------------------- | |
| NOV28g | SLEDDGLP---------------------------------------------------- | |
| NOV28h | SLEDDGLP---------------------------------------------------- | |
| NOV28i | --EDSKMGSTVTAAVIGIIVPIVVIALLCMSGYLIWRNWKRKNTKSMNFDNPVYRKTTEE | |
| NOV28 | SLEDDGLP---------------------------------------------------- | |
| NOV28k | --EDSKMGSTVTAAVIGIIVPIVVIALLCMSGYLIWRNWKRKNTKSMNFDNPVYRKTTEE | |
| NOV28l | SLEDDGLP---------------------------------------------------- | |
| NOV28a | ------------------------------------------------------------ | |
| NOV28b | ------------------------------------------------------------ | |
| NOV28d | ------------------------------------------------------------ | |
| NOV28e | ------------------------------------------------------------ | |
| NOV28f | ------------------------------------------------------------ | |
| NOV28g | ------------------------------------------------------------ | |
| NOV28h | ------------------------------------------------------------ | |
| NOV28i | EDEDELHIGRTAQIGHVYPAAISSFDRPLWAEPCLGETREPEDPAPALKELFVLPGEPRS | |
| NOV28 | ------------------------------------------------------------ | |
| NOV28k | EDEDELHIGRTAQIGHVYPAAISSFDRPLWAEPCLGETREPEDPAPALKELFVLPGEPRS | |
| NOV28l | ------------------------------------------------------------ | |
| NOV28a | ------------------------------- | |
| NOV28d | ------------------------------- | |
| NOV28e | ------------------------------- | |
| NOV28f | ------------------------------- | |
| NOV28g | ------------------------------- | |
| NOV28h | ------------------------------- | |
| NOV28i | QLHQLPKNPLSELPVVKSKRVALSLEDDGLP | |
| NOV28 | ------------------------------- | |
| NOV28k | QLHQLPKNPLSELPVVKSKRVALSLEDDGLP | |
| NOV28l | ------------------------------- | |

Further analysis of the NOV28a protein yielded the following properties shown in Table 28C.

TABLE 28C
Protein Sequence Properties NOV28a

| | |
|---|---|
| SignalP analysis: | Cleavage site between residues 33 and 34 |
| PSORT II analysis: | |

| PSG: | a new signal peptide prediction method | |
|---|---|---|
| | N-region: | length 10; pos. chg 1; neg. chg 1 |
| | H-region: | length 14; peak value 11.70 |
| | PSG score: | 7.30 |

| | |
|---|---|
| GvH: | von Heijne's method for signal seq. recognition |
| | GvH score (threshold: −2.1): 4.70 |
| | possible cleavage site: between 26 and 27 |

>>> Seems to have a cleavable signal peptide (1 to 26)

| ALOM: | Klein et al's method for TM region allocation | | |
|---|---|---|---|
| | Init position for calculation: 27 | | |
| | Tentative number of TMS(s) for the threshold 0.5: 1 | | |
| | Number of TMS(s) for threshold 0.5: 1 | | |
| | INTEGRAL | Likelihood = | Transmembrane 768-784 |
| | | −13.80 | |

| | |
|---|---|
| PERIPHERAL | Likelihood = 2.81 (at 203) |

| | |
|---|---|
| | ALOM score: −13.80 (number of TMSs: 1) |
| MTOP: | Prediction of membrane topology (Hartmann et al.) |
| | Center position for calculation: 13 |
| | Charge difference: −0.5 C(0.5)-N(1.0) |
| | N >= C: N-terminal side will be inside |

>>> membrane topology: type 1a (cytoplasmic tail 785 to 905)

| MITDISC: | discrimination of mitochondrial targeting seq | | | |
|---|---|---|---|---|
| | R content: | 2 | Hyd Moment(75): | 5.68 |
| | Hyd Moment(95): | 4.45 | G content: | 2 |
| | D/E content: | 2 | S/T content: | 0 |

| | |
|---|---|
| | Score: −6.43 |
| Gavel: | prediction of cleavage sites for mitochondrial preseq |
| | R-2 motif at 35 LRL\|QH |

NUCDISC: discrimination of nuclear localization signals

| | |
|---|---|
| | pat4: none |
| | pat7: PVVKSKR (3) at 888 |
| | bipartite: none |
| | content of basic residues: 10.3% |
| | NLS Score: −0.22 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 89 |
| COIL: | Lupas's algorithm to detect coiled-coil regions |
| | total: 0 residues |

Final Results (k = 9/23):

44.4%: extracellular, including cell wall
22.2%: Golgi
22.2%: endoplasmic reticulum
11.1%: plasma membrane

>> prediction for CG55256-07 is exc (k = 9)

A search of the NOV28a protein against the Geneseq database, a proprietary database that contains sequences published in patents and patent publication, yielded several homologous proteins shown in Table 28D.

TABLE 28D
Geneseq Results for NOV28a

| | | NOV28a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU91290 | Human NOV5i protein - Homo sapiens, | 1 . . . 905 | 903/905 (99%) | 0.0 |
| | 905 aa. [WO200216600-A2, 28-FEB-2002] | 1 . . . 905 | 903/905 (99%) | |
| AAU91288 | Human NOV5g protein - Homo sapiens, 857 aa. | 1 . . . 905 | 851/905 (94%) | 0.0 |
| | [WO200216600-A2, 28-FEB-2002] | 1 . . . 857 | 854/905 (94%) | |
| ABP56838 | Human apolipoprotein E receptor 2 protein | 1 . . . 905 | 800/973 (82%) | 0.0 |
| | SEQ ID NO: 5 - Homo sapiens, | 1 . . . 963 | 817/973 (83%) | |
| | 963 aa. [WO200299438-A2, 12-DEC-2002] | | | |
| AAU91289 | Human NOV5h protein - Homo sapiens, | 1 . . . 905 | 786/908 (86%) | 0.0 |
| | 847 aa. [WO200216600-A2, 28-FEB-2002] | 1 . . . 847 | 802/908 (87%) | |
| AAU91285 | Human NOV5d protein - Homo sapiens, | 42 . . . 905 | 704/884 (79%) | 0.0 |
| | 1012 aa. [WO200216600-A2, 28-FEB-2002] | 162 . . . 1012 | 724/884 (81%) | |

In a BLAST search of public sequence databases, the NOV28a protein was found to have homology to the proteins shown in the BLASTP data in Table 28E.

TABLE 28E
Public BLASTP Results for NOV28a

| | | NOV28a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| Q14114 | Apolipoprotein E receptor 2 | 1 . . . 905 | 800/973 | (82%) | 0.0 |
| | precursor - Homo sapiens | 1 . . . 963 | 817/973 | (83%) | |
| | (Human), 963 aa. | | | | |
| Q921B6 | ApoE receptor-2 - Mus musculus | 1 . . . 905 | 719/912 | (78%) | 0.0 |
| | (Mouse), 870 aa. | 1 . . . 870 | 766/912 | (83%) | |
| O14968 | ApoER2delta4-7 - Homo sapiens | 1 . . . 905 | 721/907 | (79%) | 0.0 |
| | (Human), 793 aa. | 1 . . . 793 | 736/907 | (80%) | |
| Q924X6 | Apolipoprotein E receptor 2 | 1 . . . 905 | 689/1011 | (68%) | 0.0 |
| | precursor - Mus musculus | 1 . . . 996 | 743/1011 | (73%) | |
| | (Mouse), 996 aa. | | | | |
| JE0237 | apolipoprotein E receptor 2 | 1 . . . 905 | 689/1009 | (68%) | 0.0 |
| | precursor- mouse, 996 aa. | 1 . . . 996 | 742/1009 | (73%) | |

PFam analysis predicts that the NOV28a protein contains the domains shown in the Table 28F.

TABLE 28F
Domain Analysis of NOV28a

| | | Identities/ | |
|---|---|---|---|
| | | Similarities | |
| | NOV28a Match | for the | Expect |
| Pfam Domain | Region | Matched Region | Value |
| Idl_recept_a | 45 . . . 83 | 20/43 (47%) | 7.7e−13 |
| | | 32/43 (74%) | |
| Idl_recept_a | 84 . . . 124 | 19/43 (44%) | 2.8e−13 |
| | | 32/43 (74%) | |
| Idl_recept_a | 125 . . . 163 | 19/43 (44%) | 1.2e−11 |
| | | 32/43 (74%) | |
| Idl_recept_a | 165 . . . 207 | 20/44 (45%) | 1.3e−07 |
| | | 33/44 (75%) | |
| EGF | 282 . . . 316 | 14/47 (30%) | 9e−06 |
| | | 27/47 (57%) | |
| TIL | 273 . . . 322 | 13/67 (19%) | 0.33 |
| | | 29/67 (43%) | |
| EGF | 322 . . . 356 | 15/47 (32%) | 0.5 |
| | | 27/47 (57%) | |
| Idl_recept_b | 404 . . . 449 | 17/48 (35%) | 1.2e−10 |
| | | 40/48 (83%) | |
| Idl_recept_b | 451 . . . 492 | 23/47 (49%) | 2.3e−15 |
| | | 40/47 (85%) | |
| Idl_recept_b | 494 . . . 536 | 19/47 (40%) | 2e−15 |
| | | 38/47 (81%) | |
| Idl_recept_b | 538 . . . 581 | 21/47 (45%) | 2.9e−12 |
| | | 39/47 (83%) | |
| Idl_recept_b | 582 . . . 622 | 12/47 (26%) | 1.5e−08 |
| | | 37/47 (79%) | |

### Example 29

The NOV29 clone was analyzed, and the nucleotide and encoded polypeptide sequences are shown in Table 29A.

TABLE 29A
NOV29 Sequence Analysis

| | | |
|---|---|---|
| NOV29a, CG55776-01 | SEQ ID NO:665 | 7876 bp |
| DNA Sequence | ORF Start: ATG at 6 | ORF Stop: TGA at 7857 |

TCAGGATGAAGGTAAAAGGCAGAGGAATCACCTGCTTGCTGGTCTCCTTTGCTGTGATCTGCCTGGTC
GCCACCCCTGGGGGCAAGGCCTGTCCTCGCCGCTGTGCCTGTTATATGCCTACGGAGGTACACTGCAC
ATTTCGGTACCTGACTTCCATCCCAGACAGCATCCCGCCCAATGTGAACGCAATGAATTTAGGGTACA
ACAGCTTGGTTAGATTGATGGAAACAGATTTTTCTGGCCTGACCAAACTGGAGTTACTCATGCTTCAC
AGCAATGGCATTCACACAATCCCTGACAAGACCTTCTCAGATTTGCAGGCCTTGCAGGTGAGACTGAT
GGTCTTAAAAATGAGCTATAATAAAGTCCGAAAACTTCAGAAAGATACTTTTTATGGCCTCAGGAGCT
TGACACGATTGCACATGGACCACAACAATATTGAGTTTATAAACCCAGAGGTTTTTTATGGGCTCAAC
TTTCTCCGCCTGGTGCACTTGGAAGGAAATCAGCTCACTAAGCTCCACCCAGATACATTTGTCTCTTT
GAGCTACCTCCAGATATTTAAAATCTCTTTCATTAAGTTCCTATACTTGTCTGATAACTTCCTGACCT
CCCTCCCTCAAGAGATGGTCTCCTATATGCCTGACCTAGACAGCCTTTACCTGCATGGAAACCCATGG
ACCTGTGATTGCCATTTAAAGTGGTTGTCTGACTGGATACAGGAGAAGCCAGGTATCTATATTGTNTT
ACCAGATGTAATAAAATGCAAAAAAGATAGAAGTCCCTCTAGTGCTCAGCAGTGTCCACTTTGCATGA
ACCCTAGGACTTCTAAAGGCAAGCCGTTAGCTATGGTCTCAGCTGCAGCTTTCCAGTGTACAAAGCCA
ACCATTGACTCATCCCTGAAATCAAAGAGCCTGACTATTCTGGAAGACAGTAGTTCTGCTTTCATCTC
TCCCCAAGGTTTCATGGCACCCTTTGGCTCCCTCACTTTGAATATGACAGATCAGTCTGGAAATGAAG
CTAACATGGTCTGCAGTATTCAAAAGCCCTCAAGGACATCACCCATTGCATTCACTGAAGAAAATGAC
TACATCGTGCTAAATACTTCAATTTTCACATTTTTGGTGTGCAACATAGATTACGGTCACATTCAGCC
AGTGTGGCAAAATTTTGGCTTTGTACAGTGATTCTCCTCTGATACTAGAAAGGAACACTTGCTTAGTG
AAACACCGCAGCTCTATTACAAATATAAACAGGTGGCTCCTAAGCCTGAAGACATTTTTACCAACATA
GAGGCAGATCTCAGAGCAGATCCCTCTTGGTTAATGCAAGACCAAATTTCCTTGCAGCTGAACAGAAC
TGCCACCACATTCAGTACATTACAGATCCAGTACTCCAGTGATGCTCAAATCACTTTACCAAGAGCAG
AGATGAGGCCAGTGAAACACAAATGGACTATGATTTCAAGGGATAACAATACTAAGCTGGAACATACT
GTCTTGGTAGGTGGAACCGTTGGCCTGAACTGCCCAGGCCAAGGAGACCCCACCCCACACGTGGATTG
GCTTCTAGCTGATGGAAGTAAAGTGAGAGCCCCTTATGTCAGTGAGGATGGACGGATCCTAATAGACA
AAAGTGGAAAATTGGAACTCCAGATGGCTGATAGTTTTGACACAGGCGTATATCACTGTATAAGCAGC
AATTATGATGATGCAGATATTCTCACCTATAGGATAACTGTGGTAGAACCTTTGGTCGAAGCCTATCA
GGAAAATGGGATTCATCACACAGTTTTCATTGGTGAAACACTTGATCTTCCATGCCATTCTACTGGTA
TCCCAGATGCCTCTATTAGCTGGGTTATTCCAGGACAATGTGCTCTATCAGTCATCAAAGAGACAAAG
AAAGTTCTAAACAATGGCACATTAAGAATATTACAGGTCACCCCGAAAGACCAAGGTTATTATCGCTG
TGTGGCAGCCAACCCATCAGGGGTTGATTTTTTGATTTTCCAAGTTTCAGTCAAGATGAAAGGACAAA
GGCCCTTGGAGCATGATGGAGAAACAGAGGGATCTGGACTTGATGAGTCCAATCCTATTGCTCATCTT
AAGGAGCCACCAGGTGCACAACTCCGTACATCTGCTCTGATGGAGGCTGAGGTTGGAAAACACACCTC
AAGCAACAAGTAAGAGGCACACTATCGGGAATTAACACTCCAGCGACGTGGAGATTCAACACATCGAC
GTTTTAGGGAGAATAGGAGGCATTTCCCTCCCTCTGCTAGGAGAATTGACCCACAACATTGGGCGGCA
CTGTTGGAGAAAGCTAAAAAGAATGCTATGCCAGACAAGCGAGAAAATACCACAGTGAGCCCACCCCC
AGGAATTTATGGTCCCGGCCACTAAAGCTTTGAACCTTCCAGCAAGGACAGTGACTGCTGACTCCAGA
ACAATATCTGATAGTCCTATGACAAACATAAATTATGGCACAGAATTCTCTCCTGTTGTGAATTCACA
AATACTACCACCTGAAGAACCCACAGATTTCAAACTGTCTACTGCTATTAAACTACAGCCATGTCAAA
AGAATATAAACCCAACCATGTCAAGCCAAATACAAGGCACAACCAATCAACATTCATCCACTGTCTTT
CCACTGCTACTTGGAGCAAACTGAATTTCAGGACTCTGACCAGATGGGAAGAGGAGAGAGCATTTCCA
AAGTAGACCCCCAATAACAAGTAAGACTATGATCAAAGATGTCAATGTCAAAATGCTTAGTAGCACCA
CCAACAAACTATTATTAGAGTCAGTAAATACCACAAATAGTCATCAGACATCTGTAAGAGAAGTGAGT
GAACCCAGGCACAATCACTTCTATTCTCACACTACTCAAATACTTAGCACCTCCACGTTCCCTTCAGA
TCCACACACAGCTGCTCATTCTCAGTTTCCGATCCCTAGAAATAGTACAGTTAACATCCCGCTGTTCA
GACGCTTTGGGAGGCAGAGGAAAATTGGCGGAAGGGGGCGGATTATCAGCCCATATAGAACTCCAGTT
CTGCGACGGCATAGATACAGCATTTTCAGGTCAACAACCAGAGGTTCTTCTGAAAAAAGCACTACTGC
ATTCTCAGCCACAGTGCTCAATGTGACATGTCTGTCCTGTCTTCCCAGGGAGAGGCTCACCACTGCCA
CAGCAGCATTGTCTTTTCCAAGTGCTGCTCCCATCACCTTCCCCAAAGCTGACATTGCTAGAGTCCCA
TCAGAAGAGTCTACAACTCTAGTCCAGAATCCACTATTACTACTTGAGAACAAACCCAGTGTAGAGAA
AACAACACCCACAATAAAATATTTCAGGACTGAAATTTCCCAAGTGACTCCAACTGGTGCAGTCATGA
AGCACCAATGAAGCTAAAAGAGATTCAGTGATTACATCGTCACTTTCAGGTGCTATCACCAAGCCACC
ATTGACTATTATAGCCATTACAAGGTTTTCAAGAAGGAAAATTCCCTGGCAACAGAACTTTGTAAATA
ACCATAACCCAAAAGGCAGATTAAGGAATCAACATAAGTTAGTTTACAAAAAAGCACAACATGTGATG
CTTCCTAAAACATCTCCTGCTTTACCACAGAGACAAAGTCTCCCCTCGCACCACACTACGACCAAAAC
ACACAATCCTGGAAGTCTTCCAACAAAGAAGGAGCTTCCCTTCCCACCCCTTAACCCTATGCTTCCTA
GTATTATAAGCAAAGACTCAAGTACAAAAAGCATCATATCAACGCAAACAGCAATACCAGCAACAACT
CCTACCTTCCCTGCATCTGTCATCACTTATGAAACCCAAACAGAGAGATCTAGAGCACAAACAATACA
TCACTACACCCACTGCTATGACACCTCCTGTTCTAACCACAGCCGAAACTTCAGTCAAGCCCAGTGTC
TCTGCATTCACTCATTCCCCACCAGAAAACACAACTGGGATTTCAAGCACAATCAGTTTTCATTCAAG
AACTCTTAATCTGACAGATGTGATTGAAGAACTAGCCCAAGCAAGTACTCAGACTTTGAAGAGCACAA
TTGCTTCTGAAACAACTTTGTCCAGCAAATCACACCAGAGTACCAACACTAGGAAAGCAATCATTAGA
CACTCAACCATACCACCATTCTTGAGCAGCAGTGCTACTCTAATGCCAGTTCCCATCTCCCCTCCCTT
TACTCAGAGAGCAGTTACTGACAACGTGGCGACTCCCATTTCCGGGCTTATGACAAATACAGTGGTCA
AAGGTTCACCCAAATGCCAAGTTCACAATTGGAACCACTCACTTCATCTACTCTAATCTGTTACATTC
TACTCCCATGCCAGCACTAACAACAGTTAAATCACAGAATTCTAAATTAACTCCATCTCCCTGGGCAG
AAAACCAATTTTGGCACAAACCATACTCAGAAATTGCTGAAAAAGGCAAAAAGCCAGAAGTAAGCATG
TTGGCTACTACAGGCCTGTCCGAGGCCACCACTCTTGTTTCAGATTGGGATGGACAGAAGAACACAAA
GAAGAGTGACTTTGATAAGAAACCAGTTCAAGAAGCAACAACTTCCAAACTCCTTCCCTTTGACTCTT
TGTCTAGGTATATATTTGAAAAGCCCAGGATAGTTGGAGGAAAAGCTGCAAGTTTTACTATTCCAGCT
AACTCAGATGCCTTTCTTCCCTGTGAAGCTGTTGGAAATCCCCTGCCCACCATTCATTGGACCAGAGT
CCCATCAGGTATGTCAGGACTTGATTTATCTAAGAGGAAACAGAATAGCAGGGTCCAGGTTCTCCCCA
ATGGTACCCTGTCCATCCAGAGGGTGGAAATTCAGGACCGCGGACAGTACTTGTGTTCCGCATCCAAT
CTGTTTGGCACAGACCACCTTCATGTCACCTTGTCTGTGGTTTCCTATCCTCCCAGGATCCTGGAGAG
ACGTACCAAAGAGATCACAGTTCATTCCGGAAGCACTGTGGAACTGAAGTGCAGAGCAGAAGGTAGGC
CAAGCCCTACAGTTACCTGGATTCTTGCAAACCAAIACAGTTGTCTCAGAATCATCCCAGGGAGTAGG
CAGGCTGTGGTGACGGTTGACGGAACATTGGTCCTCCACAATCTCAGTATTTATGACCGTGGCTTTTA
CAAATGTGTGGCCAGCAACCCAGGTGGCCAGGATTCACTGCTGGTTAAAATACAAGTCATTGCAGCAC
CACCTGTTATTCTAGAGCAAAGGAGGCAAGTCATTGTAGGCACTTGGGGTGAAAGTTTAAAACTGCCC
TGTACTGCAAAGGAACTCCTCAGCCCAGCGTTTACTGGGTCCTCTCTGATGGCACTGAAGTGAAAACC
ATTACAGTTTACCAATTCCAAGTTGTTCTTATTTTCAAATGGGACTTTGTATATAAGAAACCTAGCCT
CTTCAGACAGGGGCACTTATGAATGCATTGCTACCAGTTCCACTGATTCGGAGCGAAGAGTAGTAATG
CTTACAATGGAAGAGCGAGTGACCAGCCCCAGGATAGAAGCTGCATCCCAGAAAGGACTGAAGTGAAA
TTTTGGGGACAAAATTACTACTGAACTGCTCAGCCACTGGGGAGCCCAAACCCAAATAATGTGGAGGT
TACCATCCAAGGCTGTGGTCGACCAGCAGCATAGGGTGGGCAGCTGGATCCACGTCTACCCTTATGGA
TCCCTGTTTATTGGATCAGTAACAGAAAAAGACAGTGGTGTCTACTTGTGTGTGGCAAGAAACAAAAT
GGGGGAATGATCTGATACTGATGCATGTTAGCCTAAGACTGAPAACCTGCCAATTGACCACAAGCAGT
ATTTTAGAAAGCAAGTGCTCCATGGGAAAGATTTCCAAGTAGATTGCAAAGCTTCCGGCTCCCCAGTG
CCAGAGATATCTTGGAGTTTGCCTGATGGAACCATGATCAACAATGCAATGCAAGCCGATGACAGTGG
CCACAGGACTAGGAGATATACCCTTTTCAACAATGGAACTTTATACTTCAACAAAGTTGGGGTAGCGG
AGGAAGGAGATTATACTTGCTATGCCCAGAACACCCTAGGGAAGATGAAATGAAAGGTCCACTTAACA
GTTATAACAGCTGCTCCCCGGATAAGGCAGAGTAACAACCAAACAAGAGAATCAAAAGCTGGAGACAC
ACATGATTTCCTTCTCCATTGATAGGTACACATTTCATGCCAAATGGGTCTTTGACCAATCAACAGTG
AAACTGCTCGATTCTGGAGAGTACGTATGTGTAGCCCGAAATCCCAGTGGGGATGACACCAAAATGTA
CAAACTGGATGTGGTCTCTAAACCTCCATTAATCAATGGTCTGTATACAAACAGAACTGTTATTAAAG
CCACAGCTGTGAGACATTCCAAAAACACTTTGACTGCAGAGCTGAAGGGACACCATCTCCTGAAAGTC
ATGTGGATCATGCCAGACAATATTTTCCTCACAGCCCCATACTATGGAAGCAGAATCACAGTCCATAA
AAATGGAACCTTGGAAATTAGGAATGTGAGGCTTTCAGATTCAGCCGACTTTATCTGTGTGGCCCGAA
ATGAAGGTGGAGAGAGCGTGTTGGTAGTACAGTTAGAAGTACTGGAAATGCTGAGAAGACCGACATTT
AGAAATCCATTTAATGAAAAAATAGTTGCCCAGCTGGGAAAGTCCACAGCATTGAATTGCTCTGTTGA
TGGTAACCCACCACCTGAAATAATCTGGATTTTACCAAATGGCACACGATTTTCCAATGGACCACAAA
GTTATCAGTATCTGATAGCAAGCAATGGTTCTTTTATCATTTCTAAAACAACTCGGGAGGATGCAGGA
AAATATCGCTGTGCAGCTAGGAATAAAGTTGGCTATATTGAGAAATTAGTCATATTAGAAATTGGCCA
GAAGCCAGTTATTCTTACCTATGCACCAGGGACAGTAAAAGGCATCAGTGGAGAATCTCTATCACTGC
ATTGTGTGTCTGATGGAATCCCTAAGCCAAATATCAAATGGACTATGCCAAGTGGTTATGTAGTAGAC
AGGCCTCAAATTAATGGGAAATACATATTGCATGACAATGGCACCTTAGTCATTAAAGAAGCAACAGC
TTATGACAGAGGAAACTATATCTGTAAGGCTCAAAATAGTGTTGGTCATACACTGATTACTGTTCCAG
TAATGATTGTAGCCTACCCTCCCCGAATTACAAATCGTCCACCCAGGAGTATTGTCACCAGGACAGGG
GCAGCCTTTCAGCTCCACTGTGTGGCCTTGGGAGTTCCCAAGCCAGAAATCACATGGGAGATGCCTGA
CCACTCCCTTCTCTCAACGGCAAGTAAAGAGAGGACACATGGAAGTGAGCAGCTTCACTTACAAGGTA
CCCTAGTCATTCAGAATCCCCAAACCTCCGATTCTGGGATATACAAATGCACAGCAAAGAACCCACTT
GGTAGTGATTATGCAGCAACGTATATTCAAGTAATCTGACATGAAATAATAAAGTC

| | | | |
|---|---|---|---|
| NOV29a, CG55776-01 | SEQ ID NO:666 | 2617 aa | MW at 290200.2 kD |
| Protein Sequence | | | |

MKVKGRGITCLLVSFAVICLVATPGGKACPRRCACYMPTEVHCTFRYLTSIPDSIPPNVERINLGYNS
LVRLMETDFSGLTKLELLMLHSNGIHTIPDKTFSDLQALQVRLMVLKMSYNKVRKLQKDTFYGLRSLT
RLHNDHIANIEFINPEVFYGLNFLRLVHLEGNQLTKLHPDTFVSLSYLQIFKSFIKFLYLSDNFLTSL
PQEMVSYMPDLDSLYLHGNPWTCDCHLKWLSDWIQEKPGIYIVLPDVIKCKKDRSPSSAQQCPLCMNP
RTSKGKPLAMVSAAAFQCAKPTIDSSLKSKSLTILEDSSSAFISPQGFMAPFGSLTLNNTDQSGNEAN
MVCSIQKPSRTSPIAFTEENDYIVLNTSFSTFLVCNIDYGHIQPVWQILALYSDSPLILERSHLLSET
PQLYYKYKQVAPKPEDIFTNIEADLRADPSWLMQDQISLQLNRTATTFSTLQIQYSSDAQITLPRAEM
RPVKHKWTMISRDNNTKLEHTVLVGGTVGLNCPGQGDPTPHVDWLLADGSKKAAPYVSEDGRILIDKS
GKLELQMADSFDTGVYHCISSNYDDADILTYRITVVEPLVEAYQENGIHHTVFIGETLDLPCHSTGIP
DASISWVIPGNNVLYQSSRDKKVLNNGTLRTLQVTPKDQGYYRCVAPAPSGVDFLIFQVSVKMKGQRP
LEHDGETEGSGLDESNPIAHLKEPPGAQLRTSALMEAEVGKHTSSTSKRHNYRELTLQRRGDSTHRRF
RENRRHFPPSARRIDPQHWAALLEKAKKNAMPDKRENTTVSPPPVVTQLPNIPGEEDDSSGMLALHEE
FMVPATKALNLPARTVTADSRTISDSPMTNINYGTEFSPVVNSQILPPEEPTDFKLSTAIKTTAMSKN
INPTMSSQIQGTTNQHSSTVFPLLLGATEFQDSDQMGRGREHFQSRPPITVRTMIKDVNVKMLSSTTN
KLLLESVNTTNSHQTSVREVSEPRHNHFYSHTTQILSTSTFPSDPHTAAHSQFPIPRNSTVNIPLFRR
FGRQRKIGGRGRIISPYRTPVLPRHRYSIFRSTTRGSSEKSTTAFSATVLNVTCLSCLPRERATTATA
ALSFPSAAPITFPKADIARVPSEESTTLVQNPLLLLENKPSVEKTTPTIKYFRTEISQVTPTGAVMTY
APTSIPMEKTHKVNASYPRVSSTNEAKRDSVITSSLSGAITKPPMTIIAITRFSRRKIPWQQNFVNNH
NPKGRLRNQHKVSLQKSTAVMLPKTSPALPQRQSLPSHHTTTKTHNPGSLPTKKELPFPPLNPMLPSI
ISKDSSTKSIISTQTAIPATTPTFPASVITYETQTERSRAQTIQREQEPQKKNRTDPNISPDQSSGFT
TPTAMTPPVLTTAETSVKPSVSAFTHSPPENTTGISSTISFHSRTLNLTDVIEELAQASTQTLKSTIA
SETTLSSKSHQSTTTRKAIIRHSTIPPFLSSSATLMPVPISPPFTQRAVTDNVATPISGLMTNTVVKL
HESSRHNAKPQQLVAEVATSPKVHPNAKFTIGTTHFIYSNLLHSTPMPALTTVKSQNSKLTPSPWAEN
QFWHKPYSEIAEKGKKPEVSMLATTGLSEATTLVSDWDGQKNTKKSDFDKKPVQEATTSKLLPFDSLS
RYIFEKPRIVGGKAASFTIPANSDAFLPCEAVGNPLPTIHWTRVPSGMSGLDLSKRKQNSRVQVLPNG
TLSIQRVEIQDRGQYLCSASNLFGTDHLHVTLSVVSYPPRILERRTKEITVHSGSTVELKCRAEGRPS
PTVTWILANQTVVSESSQGSRQAVVTVDGTLVLHNLSIYDRGFYKCVASNPGGQDSLLVKIQVIAAPP
VILEQRRQVIVGTWGESLKLPCTAKGTPQPSVYWVLSDGTEVKPLQFTNSKLFLFSNGTLYIAILASS
DRGTYECIATSSTGSERRVVMLTMEERVTSPRIEAASQKRTEVNFGDKLLLNCSATGEPKPQIMARLP
SKAVVDQQHRVGSWIHVYPNGSLFIGSVTEKDSGVYLCVARNKMGDDLILMHVSLRLKPAKIDHKQYF
RKQVLHGKDFQVDCKASGSPVPEISWSLPDGTMINNAMQADDSGHRTRRYTLFNNGTLYFNKVGVAEE
GDYTCYAQNTLGKDEMKVHLnTITAAPRIRQSNKTNKRIKAGDTAVLDCEVTGDPKPKIFWLLPSNDM
ISFSIDRYTFHANGSLTINKNKLLDSGEVCVARNPSGDDDTKMYKLDVVSKPPLINGLYTNRTVIKAT
AVRHSKKHFDCRAEGTPSPEVMWIMPDNIFLTAPYYGSRITVHKNGTLEIRNVRLSDSADFICVARNE
GGESVLVVQLEVLAEMLRRPTFRNPFNEKMAQLGKSTALNCSVDGNPPPEIIWILPNGTRFSNGPQSY
QYLIASNGSFIISKTTREDAGKYRCAARNKVGYIEKLVILEIGQKPVILTYAPGTVKGISGESLSLHC
VSDGIPKPNIKWTMPSGYVVDRPQINGKYILHDNGTLVIKEATAYDRGNYICKAQNSVGHTLITVPVM
IVAYPPRITRPPRSIVTRTGAAFQLHCVALAGVPKPEITWEMPDHSLLSTASKERTHGSEQLHLQGTL
VIQNPQTSDSGIYKCTAKNPLGSDYAATYIQVI

| | | |
|---|---|---|
| NOV29b, 248210167 | SEQ ID NO:667 | 475 bP |
| DNA Sequence | ORF Start: at 2 | ORF Stop: end of Sequence |

CACCCTCGAGAATGTGGAACGCATCAATTTAGGGTACAACAGCTTGGTTAGATTGATGGAAACAGATT
TTTCTGGCCTGACCAAACTGGACTTACTCATGCTTCACAGCAATGGCATTCACACAATCCCTGACAAG
AGATACTTTTTATGGCCTCAGGAGCTTGACACGATTGCACATGGACCACAACAATATTGAGTTTATAA
ACCCAGAGGTTTTTGATGGGCTCAACTTTCTCCGCCTGGTGCACTTGGAAGGAAATCAGCTCACTAAG
CTCCACCCAGATACATTTGTCTCTTTGAGCTACCTCCAGATATTTAAAATCTCTTTCATTAAGTTCCT
ATACTTGTCTGATAACTTCCTGACCTCCCTCCCTCAAGAGATGGTCTCCTATATGCCTCTCGAGGGC

| | | | |
|---|---|---|---|
| NOV29b, 248210167 | SEQ ID NO:668 | 158 aa | MW at 18415.2 kD |
| Protein Sequence | | | |

TLENVERINLGYNSLVRLMETDFSGLTKLELLMLHSNGIHTIPDKTFSDLQALQVLKMSYNKVRKLQK
DTFYGLRSLTRLHMDHNNIEFINPEVFDGLNFLRLVHLEGNQLTKLHPDTFVSLSYLQIFKISFIKFL
YLSDNFLTSLPQEMVSYMPLEG

| | | |
|---|---|---|
| NOV29c, 247679561 | SEQ ID NO:669 | 2011 bP |
| DNA Sequence | ORF Start: at 2 | ORF Stop: end of Sequence |

AGGCTCCGCGGCCGCCCCCTTCACCCTCGAGGGGGACAATTACTACTGAAACTGCTCAGCCACTGGGG
AGCCCAAACCCCAAATAATGTGGAGGTTACCATCCAAGGCTGTGGTCGACCAGCAGCATAGAGTGGGC
AGCTGGATCCACGTCTACCCTAATGGATCCCTGTTTATTGGATCAGTACAGAAAAAAGACAGTGGTGT
CTACTTGTGTGTGGCAAGAAACAAAATGGGGGATGATCTGATACTGATGCATGTTAGCCTAAGACTGA
AACCTGCCAAAATTGACCACAAGCAGTATTTTAGAAAGCAAGTGCTCCATGGGAAAGATTTCCAAGTA
GATTGCAAAGCTTCCGGCTCCCCAGTGCCAGAGATATCTTGGAGTTTGCCTGATGGAACCATGATCAA
CAATGGATGCAAGCCGATGACAGTGGCCACAGGACTAGGAGATATACCCTTTTCAAACAATGGAACTT
TATACTTCAACAAAGTTGGGGTAGCGGAGGAAGGAGATTATACTTGCTATGCCCAGAACACCCTAGGG
AAAGATGAAATGAAGGTCCACTTAACAGTTATAACAGCTGCTCCCCGGATAAGGCAGAGTAACAAAAC
CAACAAGAGAATCAAGCTGGAGACACAGCTGTCCTTGACTGTGAGGTCACTGAGATCCCAAAACCAAA
AAATATTTTGGTTGCTGCCTTCCAATGACATGATTTCCTTCTCCATTGATAGGTACACATTTCATGCC
AATGGGTCTTTGACCATCAACAAAGTGAAACTGCTCGATTCTGGAGAGTACGTATGTGTAGCCCGAAA
TCCCAGTGGGGATGACACCAATGTACAAACTGGATGTGGTCTCTAAACCTCCAATTAATCAAATGGTC
TGTATACAAACAGAACTGTTATTAAAGCCACAGCTGTGAGACATTCCAAAAAACACTTTGACTGCAGA
GCTGAAGGGACACCATCTCCTGAAGTCATGTGGATCATGCCAGACAATATTTTCCTCACAGCCCCATA
CTATGGAAGCAGAATCACAGTCCATAAAAATGGAACCTTGGAAATTAGGAATGTGAGGCTTTCAGATT
CAGCCGACTTTATCTGTGTGGCCCGAAATGAAGGTGGAGAGAGCGTGTTGGTAGTACAGTTAGAAGTA
CTGGAAATGCTGAGAAGACCGACATTTAGAAATCCATTTAATGAAAAAATAGTTGCCCAGCTGGGAAA
GTCCACAGCATTGAATTGCTCTGTTGATGGTAACCCACCACCTGAAATAATCTGGATTTTACCAAATG
GCACACGATTTTCCAATGGACCACAAAGTTATCAGTATCTGATAGCAAGCAATGGTTCTTTTATCATT
TCTAAAACAACTCGGGAGGATGCAGGAAATATCGCTGTGCAGCTAGGAATAAAGTTGGCTATATTGAA
GAAATTAGTCATATTAGAAATTGGCCAGAAGCCAGTTATTCTTACCTATGCACCAGGGACAGTAAAAG
GCATCAGTGGAGAATCTCTATCACTGCATTGTGTGTCTGATGGAATCCCTAAGCCAAATATCAAATGG
ACTATGCCAAGTGGTTATGTAGTAGACAGGCCTCAAATTAATGGGAAATACATATTGCATGACAATGG
CACCTTAGTCATTAAAGAAGCAACAGCTTATGACAGAGGAACTATATCTGTAAGGCTCAAAAAAAGTG
TTGGTCATACACTGATTACTGTTCCAGTAATGATTGTAGCCTACCCTCCCCGAATTACAATCGTCCAA
CCCAGGAGTATTGTCACCAGGACAGGGGCAGCCTTTCAGCTCCACTGTGTGGcCTTGGGAGTTCCCAA
GCCAGAATCACATGGGAQATGCCTGACCACTCCCTTCTCTCAAACGGCAAAGTAAGAGAGGACACATG
GAAAGTGAGCAGCTTCACTTACAAGGTACCCTAGTCATTCAGATCCCCAAAQCTCCGATTCTGGGATA
TACAAATGCACAGCACTCGAGGGCAAGGGTGGGCGCGCC

| | | | |
|---|---|---|---|
| NOV29c, 247679561 | SEQ ID NO:670 | 670 aa | MW at 73997.1 kD |
| Protein Sequence | | | |

GSAAAPFTLEGDKLLLNCSATGEPKPQIMWRLPSKAVVDQQHRVGSWIHVYPNGSLFIGSVTEKDSGV
YLCVARNKMGDDLILMHVSLRLKPAKIDHKQYFRKQVLHGKDFQVDCKASGSPVPEISWSLPDGTMIN
NAMQADDSGHRTRRYTLFNNGTLYFNKVGVAEEGDYTCYAQNTLGKDEMKVHLTVITAAPRIRQSNKT
NKRIKAGDTAVLDCEVTGDPKPKIFWLLPSNDMISFSIDRYTFHANGSLTINKVKLLDSGEYVCVARN
PSGDDTKMYKLDVVSKPPLINGLYTNRTVIKATAVRHSKKHFDCRAEGTPSPEVMWIMPDNIFLTAPY
YGSRIIVHKNGTLERIAVRLSDSADFICVARNEGGESVLVVQLEVLEMIRRPTFRNPFNEKIVAQLGK
STALNCSVDGNPPPEIIWILPNGTRFSNGPQSYQYLIASNGSFIISKTTREDAGKYRCAARNKVGYIE
KLVILEIGQKPVILTYAPGTVKGISGESLSLHCVSDGIPKPNIKWTMPSGYVVDRPQINGKYILHDNG
TLVIKATAYDRGNYICKAQNSVGHTLITVPVMIVAYPPRIITNRPPRSIVTRTGAAFQLHCVALGVPK
PEITWEMPDHSLLSTASKERTHGSEQLHLQGTLVIQNPQTSDSGIYKCTALEGKGGRA

| | | |
|---|---|---|
| NOV29d, 248057904 | SEQ ID NO:671 | 771 bP |
| DNA Sequence | ORF Start: at 1 | ORF Stop: end of Sequence |

AAGCTTGCCTGTCCTCGCCGCTGTGCCTGTTATATGCCTACGGAGGTACACTGCACATTTCGGTACCT
GACTTCCATCCCAGACAGCATCCCGCCCAATGTGGAACGCATCAATTTAGGATACAACAGCTTGGTTA
GATTGATGGAAACAGATTTTTCTGGCCTGACCAAACTGGAGTTACTCATGCTTCACAGCAATGGCATT
CACACAATCCCTGACAAGACCTTCTCAGATTTGCAGGCCTTGCAGGTCTTAAAAATGAGCTATAATAA
AGTCCGAAAACTTCAGAAAGATACTTTTTATGGCCTCAGGAGCTTGACACGATTGCACATGGACCACA
ACAATATTGAGTTTATAAACCCAGAGGTTTTTTATGGGCTCAACTTTCTCCGCCTGGTGCACTTGGAA
GGAAATCAGCTCACTAAGCTCCACCCAGATACATTTGTCTCTTTGAGCTACCTCCAGATATTTAAAAT
CTCTTTCATTAAGTTCCTATACTTGTCTGATAACTTCCTGACCTCCCTCCCTCAAGAGATGGTCTCCT
ATATGCCTGACCTAGACAGCCTTTACCTGCATAAACCCATGGAAACCTGTGATTGCCATTTAAAGTGG
TTGTCTGACTGGATACAGGAGAAGCCAGATGTATAAATGCAAAAAGAATAAGAAAGTCCCTCTAGTGC
TCAGCAGTGTCCACTTTGCATGAACCCTAGGACTTCTAAAGGCAAGCCGTTAGCTATGGTCTCAGCTG
CAGCTTTCCAGTGTGCCCTCGAG

| | | | |
|---|---|---|---|
| NOV29d, 248057904 | SEQ ID NO:672 | 257 aa | MW at 29661.2 kD |
| Protein Sequence | | | |

KLACPRRCACYMPTEVHCTFRYLTSIPDSIPPNVERINLGYNSLVRLMETDFSGLTKLELLMLHSNGI
HTIPDKTFSDLQALQVLKMSYNKVRKLQKDTFYGLRSLTRLHMDHNNIEFINPEVFYGLNFLRLVHLE
GNQLTKLHPDTFVSLSYLQIFKISFIKFLYLSDNFLTSLPQEMVSYNPDLDSLYLHGNPWTCDCHLKW
LSDWIQEKPDVIKCKKDRSPSSAQQCPLCMNPRTSKGKPLAMVSAAAFQCALE

| | | |
|---|---|---|
| NOV29e, 248057927 | SEQ ID NO:673 | 771 bP |
| DNA Sequence | ORF Start: at 1 | ORF Stop: end of Sequence |

AAGCTTGCCTGTCCTCGCCGCTGTGCCTGTTATATGCCTACGGAGGTACACTGCACATTTCGGTACCT
GACTTCCATCCCAGACAGCATCCCGCCCAATGTGGAACGCATCAATTTAGGATACAACAGCTTGGTTA
GATTGATGGAAACAGATTTTTCTGGCCTGACCAAACTGGAGTTACTCATGCTTCACAGCAATGGCATT
CACACAATCCCTGACAAGACCTTCTCAGATTTGCAGGCCTTGCAGGTCTTAAAAATGAGCTATAACAA
AGTCCGAAAACTTCAGAAAGATACTTTTTATGGCCTCAGGAGCTTGACACGATTGCACATGGACCACA
ACAATATTGAGTTTATAAACCCAGAGGTTTTTTATGGGCTCAACTTTCTCCGCCTGGTGCACTTGGAA
GGAAATCAGCTCACTAAGCTCCACCCAGATACATTTGTCTCTTTGAGCTACCTCCAGATATTTAAAAT
CTCTTTCATTAAGTTCCTATACTTGTCTGATAACTTCCTGACCTCCCTCCCTCAAGAGATGGTCTCCT
ATATGCCTGACCTAGACAGCCTTTACCTGCATGGAAACCCATGGACCTGTGATTGCCATTTAAAGTGG
TTGTCTGACTGGATACAGGAGAIAGCCAGATGTAATAAATGCAAAAAAGATAGAAGTCCCTCTAGTGC
TCAGTAGTGTCCACTTTGCATGAACCCTAGGACTTCTAAAGGCAAGCCGTTAGCTATGGTCTCAGCTG
CAGCTTTCCAGTGTGCCCTCGAG

| | | | |
|---|---|---|---|
| NOV29e, 248057927 | SEQ ID NO:674 | 257 aa | MW at 29661.2 kD |
| Protein Sequence | | | |

KLACPPRCACYMPTEVHCTFRYLTSIPDSIPPNVERINLGYNSLVRLMETDFSGLTKLELLMLHSNGI
HTIPDKTFSDLQALQVLKMSYNKVRKLQKDTFYGLRSLTRLHMDHNNIEFINPEVFYGLNFLRLVHLE
GNQLTKAHPDTFVSLSYLQIFKISFIKFLYLSDNFLTSLPQEMVSYMPDLDSLYLHGNPWTCDCHLKW
LSDWIQEKPDVIKCKKDRSPSSAQQCPLCNNPRTSKGKPLAMVSAAAFQCALE

| | | |
|---|---|---|
| NOV29f, 249239821 | SEQ ID NO:675 | 771 bP |
| DNA Sequence | ORF Start: at 1 | ORF Stop: end of Sequence |

AAGCTTGCCTGTCCTCGCCGCTGTGCCTGTTATATGCCTACGGAGGTACACTGCACATTTCGGTACCT
GACTTCCATCCCAGACAGCATCCCGCCCATGTGGAACGCATCAATTTAAGGAATAACACAATTGGTTA
GATTGATGGAAACAGATTTTTCTGGCCTGACCAAACTGGAGTTACTCATGCTTCACAGCAATGGCATT
CACACAATCCCTGACAAGACCTTCTCAGATTTGCAGGCCTTGCAGGTCTTAAATGAAGCTATAAACAA
AGTCCGAAAACTTCAGAAAGATACTTTTTATGGCCTCAGGAGCTTGACACGATTGCACATGGACCACA
ACAATATTGAGTTTATAAACCCAGAGGTTTTTGATGGGCTCAACTTTCTCCGCCTGGTGCACTTGGAA
GGAAATCAGCTCACTAAGCTCCACCCAGATACATTTGTCTCTTTGAGCTACCTCCAGATATTTAAAAT
CTCTTTCATTAAGTTCCTATACTTGTCTGATAACTTCCTGACCTCCCTCCCTCMAGAGATGGTCTCCT
ATATGCCTGACCTAGACAGCCTTTACCTGCATGGAAACCCATGGACCTGTGATTGCCATTTAAAGTGG
TTGTCTGACTGGATACAGGAGAAGCCAGATGTAATAAATGCAAAAAGATAAGAGTCCCTCTAAAGTGC
TCCGCAGTGTCCACTTTGCATGAACCCTAGGACTTCTAAAGGCAAGCCGTTAGCTATGGTCTCAGCTG
AGCTTTCCAGTGTGCCCTCGAG

| | | | |
|---|---|---|---|
| NOV29f, 249239821 | SEQ ID NO:676 | 257 aa | MW at 29582.1 kD |
| Protein Sequence | | | |

KLACPRRCACYMPTEVHCTFRYLTSIPDSIPPNVERINLGYNSLVRLMETDFSGLTKLELLMLHSNGI
HTIPDKTFSDLQALQVLKMSYNKVRKLQKDTFYGLRSLTRLHMDHINNIFINPEVFDGLNFLRLVHLE
GNQLTKLHPDTFVSLSYLQIFKISFIKFLYLSDNFLTSLPQEMVSYMPDLDSLYLHGNPWTCDCHLKW
LSDWIQEKPDVIKCKKDRSPSSAPQCPLCMNPRTSKGKPLAMVSAAAFQCALE

| | | |
|---|---|---|
| NOV29g, 248057920 | SEQ ID NO:677 | 771 bP |
| DNA Sequence | ORF Start: at 1 | ORF Stop: end of Sequence |

AAGCTTGCCTGTCCTCGCCGCTGTGCCTGTTATATGCCTACGGAGGTACACTGCACATTTCGGTACCT
GACTTCCATCCCAGACAGCATCCCGCCCAATGTGGAACGCATCAATTTAGGATACAACAGCTTGGTTA
CACACAATCCCTGACAAGACCTTCTCAGATTTGCAGGCCTTGCAGGTCTTAAAAATGAGCTATAACAA
AGTCCGAAAACTTCAGAAAGATACTTTTTATGGCCTCAGGAGCTTGACACGATTGCACATGGACCACA
ACAATATTGAGTTTATAAACCCAGAGGTTTTTGATGGGCTCAACTTTCTCCGCCTGGTGCACTTGGAA
CTCTTTCATTAAGTTCCTATACTTGTCTGATAACTTCCTGACCTCCCTCCCTCAAGAGATGGTCTCCT
ATATGCCTGACCTAGACAGCCTTTACCTGCATGGAAACCCATGGAACCTGTGATTGCCATTTAAGTGG
TTGTTTGACTGGATACAGGAGAAGCCAGATGTAATAAAATGCAAAAAAGATAGAAGTCCCTCTAGTGC
TCAGCAGTGTCCACTTTGCATGAACCCTAGGACTTCTAAAGGCAAGCCGTTAGCTATGGTCTCAGCTG
CAGCTTTCCAGTGTGCCCTCGAG

| | | | |
|---|---|---|---|
| NOV29g, 248057920 | SEQ ID NO:678 | 257aa | MW at 29673.2 kD |
| Protein Sequence | | | |

KLACPRRCACYMPTEVHCTFRYLTSIPDSIPPNVERINLGYNSLVRLMETDFSGLTKLELLMLHSNGI
HTIPDKTFSDLQALQVLAMSYNKVRKLQKDTFYGLRSLTRLHMDHNNIEFINPEVFDGLNFLRLVHLE
GNQLTKLHPDTFVSLSYLQIFKISFIKFLYLSDNFLTSLPQEMVSYMPDLDSLYLHGNPWTCDCHLKW
LFDWIQEKPDVIKCKKDRSPSSAQQCPLCAPRTSKGKPLAMVSAAAFQCALE

| | | |
|---|---|---|
| NOV29h, 247679541 | SEQ ID NO:679 | 2059 bP |
| DNA Sequence | ORF Start: at 2 | ORF Stop: end of Sequence |

AGGCTCCGCGGCCGCCCCCTTCACCCTCGAGGGGGACAAATTACTACTGAACTGCTCAGCCACTGGGG
AGCCCAAACCCCAAATAATGTGGAGGTTACCATCCAAGGCTGTGGTCGACCAGCAGCATAGAGTGGGC
AGCTGGATCCACGTCTACCCTAATGGATCCCTGTTTATTGGATCAGTAACAGAAAAAGACAGTGGTGT
CTACTTGTGTGTGGCAAGAAACAAAGGGGGATGATCTGATACTGATGCATGTTAGCCTAAGAAACTGA
AACCTGCCAAATTGACCACAAGCAGTATTTTAGAAAGCAAGTGCTCCATGGGAAAGATTTCCAAAGTA
GATTGCALAGCTTCCGGCTCCCCAGTGCCAGAGATATCTTGGAGTTTGCCTGATGGAACCATGATCAA
CAATGCAATGCAAGCCGATGACAGTGGCCACAGGACTAGGAGATATACCCTTTTCAACAATGGAACTT
TATACTTCAACAAAGTTGGGGTAAGCGGAGGAGGAGATTATACTTGCTATGCCCAGAACACCCTAGGG
AAAGATGAAATGAAGGTCCACTTAACAGTTATAACAGCTGCTCCCCGGATAAGGCAGAGTAACAAAAC
CAACAAGAGAATCAAAAGCTGGAGACACAGCTGTCCTTGACTGTGAGGTCACTGAGATCCCAAACCAA
AAATATTTTGGTTGCTGCCTTCCAATGACATGATTTCCTTCTCCATTGATAGGTACACATTTCATGCC
AATGGGTCTTTGACCATCAACAAGTGAAACTGCTCGATTCTGGAAGAGTACGTATGTGTAGCCCGAAA
TCCCAGTGGGGAATGACACCAAAATGTAACAACTGGAATGTGGTCTCTAACCTCCATTATCAATGGTC
TGTATACAAATAGAACTGTTATTAAAGCCACAGCTGTGAGACATTCCAAAAAACACTTTGACTGCAGA
GCTGAAGGGACACCATCTCCTGAAGTCATGTGGATCATGCCAGACAATATTTTCCTCACAGCCCCATA
CTATGGAAGCAGAATCACAGTCCATAAAAATGGAACCTTGGAAATTAGGAATGTGAGGCTTTCAGATT
CAGCCGACTTTATCTGTGTGGCCCGAAATGAAGGTGGAGAGAGCGTGTTGGTAGTACAGTTAGAAGTA
CTGGAAATGCTGAGAAGACCGACATTTAGAAATCCATTTAATGAAAAAATAGTTGCCCAGCTGGGAAA
GTCCACAGCATTGAATTGCTCTGTTGATGGTAACCCACCACCTGAAATATCTGGATTTTAACCAAATG
GCACACGATTTTCCAATGGACCACAAAGTTATCAGTATCTGATAGCAAGCAATGGTTCTTTTATCATT
TCTAAAACAACTCGGGAGGATGCAGGAAAATATCGCTGTGCAGCTAGGAATAAAGTTGGCTATATTGA
GAAATTAGTCATATTAGAAATTGGCCAGAAGCCAGTTATTCTTACCTATGCACCAGGGACAGTAAAAG
GCATCAGTGGAGAATCTCTATCACTGCATTGTGTGTCTGATGGAATCCCTAAGCCAAATATCAAATGG
ACTATGCCAAGTGGTTATGTAGTAGACAGGCCTCAAATTAATGGGAAATACATATTGCATGACAATGG
CACCTTAGTCATTAAAGAAGCAACAGCTTATGACAGAGGAAACTATATCTGTAAGGCTCAAAATAGTG
TTGGTCATACACTGATTACTGTTCCAGTAATGATTGTAGCCTACCCTCCCCGAATTACAAATCGTCCA
CCCAGGAGTATTGTCACCAGGACAGGGGCAGCCTTTCAGCTCCACTGTGTGGCCTTGGGAGTTCCCAA
GCCAGAAATCACGTGGGAGATGCCTGACCACTCCCTTCTCTCAACGGCAAGTAAAGAGAGGACACATG
GAAGTGAGCAGCTTCACTTACAAGGTACCCTAGTCATTCAGAATCCCCAAACCTCCGATTCTGGGATA
TACAAATGCACAGCAAAGAACCCACTTGGTAGTGATTATGCAGCAACGTATATTCAAGTAATCCTCGA
GGGCAAGGGTGGGCGCGCC

| | | | |
|---|---|---|---|
| NOV29h, 247679541 | SEQ ID NO:680 | 686 aa | MW at 75732.1 kD |
| Protein Sequence | | | |

GSAAAPFTLEGDKLLLNCSATGEPKPQIMWRLPSKAVVDQQHRVGSWIHVYPNGSLFIGSVTEKDSGV
YLCVAPNKMGDDLILMHVSLRLKPAKIDHKQYFRKQVLHGKDFQVDCKASGSPVPEISWSLPDGTMIN
NKRIKAGDTAVLDCEVTGDPKPKIFWLLPSNDMISFSIDRYTFHANGSLTINKVKLLDSGEYVCVARN
PSGDDTKMYKLDVVSKPPLINGLYTNRTVIKATAVRHSKKHFDCRAEGTPSPEVMWIMPDNIFLTAPY
YGSRITVHKNGTLEIRNVRLSDSADFICVARNEGGESVLVVQLEVLEMLRRPTFRNPFNEKIVAQLGK
STALNCSVDGNPPPEIIWILPNGTRFSNGPQSYQYLIASNGSFTISKTTREDAGKYRCAARNKVGYIE
KLVILEIGQKPVILTYAPGTVKGISGESLSLHCVSDGIPKPNIKWTMPSGYVVDRPQINGKYILHDNG
TLVIKEATAYDRGNYICKAQNSVGHTLITVPVMIVAYPPRITNRPPRSIVTRTGAAFQLHCVALGVPK
PEITWEMPDHSLLSTASKERTHGSEQLHLQGTLVIQNPQTSDSGIYKCTAIAPLGSDYAATYIQVILE
GKGGRA

| | | |
|---|---|---|
| NOV291, 249116954 | SEQ ID NO:681 | 2011 bP |
| DNA Sequence | ORF Start: at 2 | ORF Stop: end of Sequence |

AGGCTCCGCGGCCGCCCCCTTCACCCTCGAGGGGGACAAATTACTACTGAACTGCTCAGCCACTGGGG
AGCCCAAACCCCAAATAATGTGGAGGTTACCATCCAAGGCTGTGGTCGACCAGCAGCATAGAGTGGGC
AGCTGGATCCACGTCTACCCTAATGGATCCCTGTTTATTGGATCAGTAACAGAAAAAGACAGTGGTGT
CTACTTGTGTGTGGCAAGAAACAAAATGGGGGATGATCTGATACTGATGCATGTTAGCCTAAGACTGA
AACCTGCCAAATTGACCACAAGCAGTATTTTAGAAAGCAAGTGCTCCATGGGAAAGATTTCCAAAGTA
TATACTTCAACAAAGTTGGGGTAGCGGAGGAAGGAGATTATACTTGCTATGCCCAGAACACCCTAGGG
CAACAAGAGAATCAAAGCTGGAGACACAGCTGTCCTTGACTGTGAGGTCACTGGGGATCCCAAACCAA
AAATATTTTGGTTGCTGCCTTCCAATGACATGATTTCCTTCTCCATTGATAGGTACACATTTCATGCC
AATGGGTCTTTGACCATCAACAAAGTGAAACTGCTCGATTCTGGAGAGTACGTATGTGTAGCCCGAAA
TCCCAGTGGGGATGACACCAAAATGTACAAACTGGATGTGGTCTCTAAACCTCCATTAATCAATGGTC
TGTATACAAATAGAACTGTTATTAAAGCCACAGCTGTGAGACATTCCAAAAAACACTTTGACTGCAGA
GCTGAAGGGACACCATCTCCTGAAGTCATGTGGATCATGCCAGACAATATTTTCCTCACAGCCCCATA
CTATGGAAGCAGAATCACAGTCCATAAAAATGGAACCTTGGAAATTAGGAATGTGAGGCTTTCAGATT
CAGCCGACTTTATCTGTGTGGCCCGAAATGAAGGTGGAGAGAGCGTGTTGGTAGTACAGTTAGAAGTA
CTGGAAATGCTGAGAAGACCGACATTTAGAAATCCATTTAATGAAAAAATAGTTGCCCAGCTGGGAAA
GTCCACAGCATTGAATTGCTCTGTTGATGGTAACCCACCACCTGAAATAATCTGGATTTTACCAAATG
GCACACGATTTTCCAATGGACCACAGTTATCAGTATCTGATAGCAAGCAAATGGTTCTTTTATCAATT
TCTAAACAACTCGGGAGGATGCAGGAAAATATCGCTGTGCAGCTAGGAATAAAAGTTGGCTATATTGA
GAAATTAGTCATATTAGAAATTGGCCAGAAGCCAGTTATTCTTACCTATGCACCAGGGACAGTAAAAG
GCATCAGTGGAGAATCTCTATCACTGCATTGTGTGTCTGATGGAATCCCTAAGCCAAATATCAAATGG
ACTATGCCAAGTGGTTATGTACTAGACAGGCCTCAAATTAATGGGAAATACATATTGCATGACAATGG
CACCTTAGTCATTAAAGAAGCAACAGCTTATGACAGAGGAAACTATATCTGTAAGGCTCAAAATAGTG
TTGGTCATACACTGATTACTGTTCCAGTAATGATTGTAGCCTACCCTCCCCGAATTACAAATCGTCCA
CCCAGGAGTATTGTCACCAGGACAGGGGCAGCCTTTCAGCTCCACTGTGTGGCCTTGGGAGTTCCCAA
GCCAGAAATCACGTGGGAGATGCCTGACCACTCCCTTCTCTCAACGGCAAGTAAAGAGAGGACACATG
GAAGTGAGCAGCTTCACTTACAAGGTACCCTAGTCATTCAGAATCCCCAAACCTCCGATTCTGGGATA
TACAAATGCACAGCACTCGAGGGCAAGGGTGGGCGCGCC

| | | | |
|---|---|---|---|
| NOV29i, 249116954 | SEQ ID NO:682 | 670 aa | MW at 73997.1 kD |
| Protein Sequence | | | |

GSAAAPFTLEGDKLLLNCSATGEPKPQIMWRLPSKAVVDQQHRVGSWIHVYPNGSLFIGSVTEKDSGV
YLCVARNKMGDDLILMHVSLRLKPAKIDHKQYFRKQVLHGKDFQVDCKASGSPVPEISWSLPDGTMIN
NAMQADDSGHRTRRYTLFNNGTLYFNKVGVAEEGDYTCYAQNTLGKDEMKVHLTVITAAPRIRQSNKT
NKRIKAGDTAVLDCEVTGDPKPKIFWLLPSNDMTSFSIDRYTFHANGSLTINKVKLLDSGEYVCVAAA
PSGDDTKMYKLDVVSKPPLINGLYTNRTVIKATAVRHSKKHFDCRAEGTPSPEVMWTMPDNIFLTAPY
YGSRITVHINGTLEIRNVRLSDSALFICVARIEGGESVLVVQLEVLEMLRRPTFPAPFNEKIVAQLGK
STALNCSVDGNPPPEIIWILPNGTRFSNGPQSYQYLIASNGSFIISKTTREDAGKYRCAAAAKVGYIE
KLVILEIGQKPVILTYAPGTVKGISGESLSLHCVSDGIPKPNIKWTMPSGYVVDRPQINGKYILHDNG
TLVIKEATAYDRGNYICKAQNSVGHTLITVPVMIVAYPPRITNRPPRSIVTRTGAAFQLHCVALGVPK
PEITWEMPDHSLLSTASKERTHGSEQLHLQGTLVIQNPQTSDSGIYKCTALEGKGGRA

| | | |
|---|---|---|
| NOV29, 248210155 | SEQ ID NO:683 | 1420 bP |
| DNA Sequence | ORF Start: at 2 | ORF Stop: end of Sequence |

CACCCTCGAGGGGGACAAATTACTACTGAACTGCTCAGCCACTGGGGAGCCCAAACCCCAAATAATGT
GGAGGTTACCATCCAAGGCTGTGGTCGACCAGCAGCATAGGTACACATTTCATGCCAATGGGTCTTTG
ACCATCAACAAAGTGAAACTGCTCGAATTCTGGAGAGTACGTATGTGTAGCCCGAATCCCAGTGGGGA
TGACACCAAAATGTACAAACTGGATGTGGTCTCTAAACCTCCATTAATCAATGGTCTGTATACAAATA
GAACTGTTATTAAAGCCACAGCTGTGAGACATTCCAAAAACAACTTTGAACTGCAGAGCTGAGGGACA
CCATCTCCTGAAAGTCATGTGGATCATGCCAGACAATATTTTCCTCACAGCCCCATACTATGGAGCAG
AATCACAGTCCATAAAAATGGAACCTTGGAAATTAGGAATGTGAGGCTTTCAGATTCAGCCGACTTTA
AGAAGAACCGACATTTAGAAATCCATTTAATGAAAAAATAGTTGCCCAGCTGGGAAGTCCACAGCATT
GAATTGCTCTGTTGATGGTAACCCACCACCTGAAATAATCTGGATTTTACCAAATGGCACACGATTTT
ATTAGAAATTGGCCAGAAGCCAGTTATTCTTACCTATGCACCAGGGACAGTAAAAGGCATCAGTGGAG
ATCTCTATCACTGCATTGTGTGTCTGATGGAAATCCCTAAGCCAAAATATCATGGACTAAATGCCAGT
GGTTATGTAGTAGACAGGCCTCAAATTAATGGGAAATACATATTGCAATGACATGGCACCTTAGTCAT
TAAAGAAGCAACAGCTTATGACAGAGGAAACTATATCTGTAAGGCTCAAATAAGTGTTGGTCATACAC
AGTCACCAGGACAGGGGCAGCCTTTCAGCTCCACTGTGTGGCCTTGGGAGTTCCCAGCCAGAAATCAC
GTGGGAGATGCCTGACCACTCCCTTCTCTCAACGGCAAGTAAAGAGAGGACACATGGAGTGAGCAAGC
TTCACTTACAAGGTACCCTAGTCATTCAGAATCCCCAAACCTCCGATTCTGGGATATACAAATGCACA
GCAAAGAACCCACTTGGTAGTGATTATGCAGCAACGTATATTCAAGTAATCCTCGAGGGC

| | | | |
|---|---|---|---|
| NOV29, 248210155 | SEQ ID NO:684 | 473 aa | MW at 52119.3 kD |
| Protein Sequence | | | |

TLEGDKLLLNCSATGEPKPQIMWRLPSKAVVDQQHRYTFHANGSLTINKVKLLDSGEYVCVARNPSGD
DTIYKLDVVSKPPLINGLYTNRTVIKATAVRHSKKHFDCRAEGTPSPEVMWIVVPDNIFLTAPYYGSR
TVHKNGTLEIRNVRLSDSADFICVARNEGGESVLWQLEVLEMLRRPTFRIAAPFNEKIVAQLGKSTAL
NCSVDGNPPPEIIWILPNGTRFSNGPQSYQYLIASNGSFIISKTTREDAG1ARCAARNKVGYIEKLVI
LEIGQKPVILTYAPGTVKGISGESLSLHCVSDGIPKPNIKWTMPSGYVVDRPQINGKYILHDNGTLVI
KEATAYDRGNYICKAQNSVGHTLITVPVMIVAYPPRITNRPPRSIVTRTGAAFQLHCVALGVPKPEIT
WEMPDHSLLSTASKERTHGSEQLHLQGTLVIQNPQTSDSGIYKCTAKNPLGSDYAATYIQVILEG

| | | |
|---|---|---|
| NOV29k, 248213764 | SEQ ID NO:685 | 1372 bP |
| DNA Sequence | ORF Start: at 2 | ORF Stop: end of Sequence |

CACCCTCGAGGGGGACAAATTACTACTGAACTGCTCAGCCACTGGGGAGCCCAAACCCCAAATAATGT
GGAGGTTACCATCCAAGGCTGTGGTCGACCAGCAGCATAGGTACACATTTCATGCCAATGGGTCTTTG
ACCATCAACAAAGTGAAACTGCTCGAATTCTGGAAGAGTACGTATGTGTAGCCCGATCCCAGTGGGGA
TGACACCAAAATGTACAAACTGGATGTGGTCTCTAAACCTCCATTAATCAATGGTCTGTATACAAACA
GAACTGTTATTAAAGCCACAGCTGTGAGACATTCCAAAAAACACTTTGACTGCAGAGCTGAAGGGACA
CCATCTCCTGAAGTCATGTGGATCATGCCAGACAATATTTTCCTCACAGCCCCATACTATGGAAGCAG
AATCACAGTCCATAAAAATGGAACCTTGGAAATTAGGAATGTGAGGCTTTCAGATTCAGCCGACTTTA
TCTGTGTGGCCCGAAATGAAGGTGGAGAGAGCGTGTTGGTAGTACAGTTAGAAGTACTGGAAATGCTG
AGAAGACCGACATTTAGAAATCCAATTTAAATGAAATAAAGTTGCCCAGCTAGA3AGTCCACAGCATT
GAATTGCTCTGTTGATGGTAACCCACCACCTGAAATAATCTGGATTTTACCAAATGGCACACGATTTT
CCAATGGACCACAAAGTTATCAGTATCTGATAGCAAGCAATGGTTCTTTTAATCATTTCTAAAACACT
CGGGAGGATGCAGGAATATCGCTGTGCAGCTAGGAATAAAGTTGGCTATATTGAGAAAAATTAGTCAT
ATTAGAATTGGCCAGAAGCCAGTTATTCTTACCTATGCACCAAGGGACAGTAAAAGGCATCAGTGGAG
AATCTCTATCACTGCATTGTGTGTCTGATGGAATCCCTAAGCCAAATATCAAATGGACTATGCCAAGT
GGTTATGTAGTAGACAGGCCTCAAATTAATGGGAAATACATATTGCATGACAATGGCACCTTAGTCAT
TAAAGAAGCAACAGCTTATGACAGAGGAAACTATATCTGTAAGGCTCAAAATAGTGTTGGTCATACAC
TGATTACTGTTCCAGTAATGATTGTAGCCTACCCTCCCCGAATTACATCGTCCACCCAAGGAAGTATT
GTCACCAGGACAGGGGCAGCCTTTCAGCTCCACTGTGTGGCCTTGGGAGTTCCCAAGCCAGAAATCAC
ATGGGAGATGCCTGACCACTCCCTTCTCTCACGGCAAGTAAAGAGAGGACACATCGAAGTGAAGCAGC
TTCACTTACAAGGTACCCTAGTCATTCAGAATCCCCAAACCTCCGATTCTGGGATATACAAATGCACA
GCACTCGAGGGC

| | | | |
|---|---|---|---|
| NOV29k, 248213764 | SEQ ID NO:686 | 457 aa | MW at 50532.6 kD |
| Protein Sequence | | | |

TLEGDKLLLNCSATGEPKPQIMWRLPSKAVVDQQHRYTFHANGSLTINKVKLLDSGEYVCVARNPSGD
DTKMYKLDVVSKPPLINGLYTNRTVIKATAVRHHSKKHFDCRAGTPSPEVMWIMPDNIFLTAPYYGSR
ITVHKNGTLEIRNVRLSDSADFICVARNEGGESVLVVQLEVLEMLRRPTFPNPFNEKIVAQLGKSTAL
NCSVDGNPPPEIIWILPNGTRFSNGPQSYQYLIASNGSFIISKTTREDAGKYRCAARNKVGYIEKLVI
LEIGQKPVILTYAPGTVKGISGESLSLHcVSDGIPKPNIKWTMPSGYVVDRPQINGKYILHDNGTLVI
KEATAYDRGNYICKAQNSVGHTLITVPVMIVAYPPRITNRPPRSIVTRTGAAFQLHCVALGVPKPEIT
WEMPDHSLLSTASKERTHRSEQLHLQGTLVIQNPQTSDSGIYKCTALEG

| | | |
|---|---|---|
| NOV29l, 248213768 | SEQ ID NO:687 | 1383 bP |
| DNA Sequence | ORF Start: at 2 | ORF Stop: at 1382 |

CACCCTCGAGGGGGACAAATTACTACTGAACTGCTCAGCCACTGGGGAGCCCAAACCCCAAATAATGT
GGAGGTTACCATCCAAGGCTGTGGTCGACCAGCAGCATAGGTACACATTTCATGCCAATGGGTCTTTG
ACCATCAACAAAGTGAAACTGCTCGATTCTGGAGAGTACGTATGTGTAGCCCGAAATCCCAGTGGGGA
TGACACCAAATGTACAAACTGGAATGTGGTCTCTAAACCTCCATTAATCAAATGGTCTGTAATACATA
GAACTGTTATTAAAGCCACAGCTGTGAGACATTCCAAAAACACTTTGACTGCAGAGCTGAAAGGGACA
CCATCTCCTGAAGTCATGTGGATCATGCCAGACAATATTTTCCTCACAGCCCCATACTATGGAAGCAG
AATCACAGTCCATAAAAATGGAACCTTGGAAATTAGGAATGTGAGGCTTTCAGATTCAGCCGACTTTA
TCTGTGTGGCCCGAAATGAAGGTGGAGAGAGCGTGTTGGTAGTACAGTTAGAAGTACTGGAAATGCTG
AGAAGACCGACATTTAGAAATCCATTTAATGAAAAAATAGTTGCCCAGCTGGGAAAGTCCACAGCATT
GAATTGCTCTGTTGATGGTAACCCACCACCGGAAATMATCTGGATTTTACCAAATGGCACACGATTTT
CCAATGGACCACAAGTTATCAGTATCTGATAGCAAGCAATGGTTCTTTTAATCATTTCTAAACAAACT
CGGGAGGATGCAGGAAAATATCGCTGTGCAGCTAGGAATAAAGTTGGCTATATTGAGAAATTAGTCAT
ATTAGAAATTGGCCAGAAGCCAGTTATTCTTACCTATGCACCAGGGACAGTAAAAGGCATCAGTGGAG
AATCTCTATCACTGCATTGTGTGTCTGATGGAATCCCTAAGCCAAATATCAAATGGACTATGCCAAGT
GGTTATGTAGTAGACAGGCCTCAAATTAATGGGAAATACATATTGCATGACAATGGCACCTTAGTCAT
TAAAGAAGCAACAGCTTATGACAGAGGAAACTATATCTGTAAGGCTCAAAATAGTGTTGGTCATACAC
TGATTACTGTTCCAGTAATGATTGTAGCCTACCCTCCCCGAATTACAAATCGTCCACCCAGGAGTATT
GTCACCAGGACAGGGGCAGCCTTTCAGCTCCACTGTGTGGCCTTGGGAGTTCCCAAGCCAGAAATCAC
GTGGGAGATGCCTGACCACTCCCTTCTCTCAACGGCAAGTAAAGAGAGGACACATGGAAGTGAGCAGC
TTCACTTACAAGGTACCCTAGTCATTCAGAATCCCCAAACCTCCGATTCTGGGATATACAAATGCACA
GCACTCGAGGGCAAGGGCGAATC

| | | | |
|---|---|---|---|
| NOV29l, 248213768 | SEQ ID NO:688 | 460 aa | MW at 50747.8 kD |

TLEGDKLLLNCSATGEPKPQIMWRLPSKAVVDQQHRYTFHANGSLTINKVKLLDSGEYVCVARNPSGD
DTKMYKLDVVSKPPLINGLYTNRTVIKATAVRHSKKHFDCRAEGTPSPEVMWIMPDNIFLTAPYYGSR
ITVHKNGTLEIRNLVRLSDSADFICVAPNEGGESVLWQLEVLEMLRRPTFRNPFNEKIVAQLGKSTAL
NCSVDGNPPPEIIWILPNGTRFSNGPQSYQYLIASNGSFIISKTTREDAGKYRCAARNKVGYIEKLVI
LEIGQKPVILTYAPGTVKGISGESLSLHVSDGIPKPNIKWTMPSGYAATDRPQINGKYILHDNGTLVI
KEATAYDRGNYICKAQNSVGHTLITVPVAMVAYPPRITNRPPRSIVTRTGAAFQLHCVALGVPKPEIT
WEMPDHSLLSTASKERTHGSEQLHLQGTLVIQNPQTSDSGIYKCTALEGKGE

| | | |
|---|---|---|
| NOV29m, 248213772 | SEQ ID NO:689 | 1372 bP |
| DNA Sequence | ORF Start: at 2 | ORF Stop: end of Sequence |

CACCCTCGAGGGGGACAAAATTACTACTGAAACTGCTCAGCCACTGGGGAGCCCACCCCAAATAATGT
GGAGGTTACCATCCAAGGCTGTGGTCGACCAGCAGCATAGGTACACATTTCATGCCAATGGGTCGTTG
ACCATCAACAAAGTGAAACTGCTCGATTCTGGAGAGTACGTATGTGTAGCCCGAAATCCCAGTGGGGA
GAACTGTTATTAAAGCCACAGCTGTGAGACATTCCAAAAAACACTTTGACTGCAGAGCTGAAGGGACA
CCATCTCCTGAAGTCATGTGGATCATGCCAGACAATATTTTCCTCACAGCCCCATACTATGGAAGCAG
AATCACAGTCCATAAAAATGGAACCTTGGAAATTAGGAATGTGAGGCTTTCAGATTCAGCCGACTTTA
TCTGTGTGGCCCGAAATGAAGGTGGAGAGAGCGTGTTGGTAGTACAGTTAGAAGTACTGGAAATGCTG
AGAAGACCGACATTTAGAAATCCATTTAATGAAAAAATAGTTGcCCAGCTGGGAAAAGTCCACAGATT
GAATTGCTCTGTTGATGGTAACCCACCACCGGAAATAATCTGGATTTTACCAAATGGCACACGATTTT
CCAATGGACCACAAAGTTATCAGTATCTGATAGCAAGCAATGATTCTTTTATCATTTCTAAAACAACT
CGGGAGGATGCAGGAAAATATCGCTGTGCAGCTAGGAATAAAGTTGGCTATATTGAGAAATTAGTCAT
ATTAGAAATTGGCCAGAAGCCAGTTATTCTTACCTATGCAACCAGGGACAGTAAAGAATCAAGTGGAG
AATCTCTATCACTGCATTGTGTGTCTGATGGAATCCCTAAGCCAAATATCAATGGACTATGCCAAAGT
GGTTATGTAGTAGACAGGCCTCAAATTAATGGGAAATACATATTGCATGACAATGGCACCTTAGTCAT
TAAAGAAGCAACAGCTTATGACAGAGGAAACTATATCTGTAAGGCTCAAAATAGTGTTGGTCATACAC
TGATTACTGTTCCAGTAATGATTGTAGCCTACCCTCCCCGAATTACAAATCGTCCACCCAGGAGTATT
GTCACCAGGACAGGGGCAGCCTTTCAGCTCCACTGTGTGGCCTTGGGAGTTCCCAAAGCCAGAATCAC
TTCACTTACAAGGTACCCTAGTCATTCAGAATCCCCAACCTCCGATTCTGGGATATACATGCACA
GCACTCGAGGGC

| | | | |
|---|---|---|---|
| NOV29m, 248213772 | SEQ ID NO:690 | 457 aa | MW at 50433.5 kD |
| Protein Sequence | | | |

TLEGDKLLLNCSATGEPKPQIMWRLPSKAVVDQQHRYTFGSLTINKKAKAALDSGEYVCVAPANPSGD
DTKMAYKLDVVSKPPLINGLYTNRTVIKATAVRHSKKHFDCAGTPSPEVMWIMPDNIFLTAAPYYGSR
ITVKKNGTLEIRNVRISDSADFICVARNEGGESVLVVQLEVLEMLRRPTFRNPFNEKIVAQLGKSTAL
NCSVDGNPPPEIIWILPNGTRFSNGPQSYQYLIASNGSFIISKTTREDAGKYRCAARNKVGYIEKLVI
LEIGQKPVILTYAPGTVKGISGESLSLHCVSDGIPKPNIKWTMPSGYVVDRPQINGKYILHDNGTLVI
IWEMPDHSLLSTASKERTHGSEQLHLQGTLVTQNPQTSDSGTYKCTALEG

| | | |
|---|---|---|
| NOV29n, 248586774 | SEQ ID NO:691 | 1420 bP |
| DNA Sequence | ORF Start: at 2 | ORF Stop: end of Sequence |

CACCCTCGAGGGGGACAATTACTACTGAACTGCTCAGCCACTGGGGAGCCCCCCCAAAAAAATAATGT
GGAGGTTACCATCCAAGGCTGTGGTCGACCAGCAGCATAGGTACACATTTCATGCCAATGGGTCTTTG
ACCAATCAACAAAGTGAAACTGCTCGATTCTGGAGAGTACGTATGTGTAGCCCGAATCCCAGTGGGGA
TGACAACCAAATGTACAAACTGGAATGTGGTCTCTAAACCTCCAATTAAATCATGGTCTGTATACATA
GAACTGTTATTAAAGCCACAGCTGTGAGACATTCCAAAAAACACTTTGACTGCAGAGCTGAAGGGACA
AATCACAGTCCATAAAAATGGAACCTTGGAAATTAGGAATGTGAGGCTTTCAGATTCAGCCGACTTTA
TCTGTGTGGCCCGAAAATGAAAGGTGGAGAGAGCGTGTTGGTAGTACAGTTAGAAGTACTGGATGCTG
AGAAGACCGACATTTAGAAATCCATTTAATGAAAAAATAGTTGCCCAGCTGGGAAAGTCCACAGCATT
GAATTGCTCTGTTGATGGTAAACCCACCACCTGAAATAATCTGGATTTTACCAATGGCACACGATTTT
CCAATGGACCACAAAGTTATCAGTATCTGATAGCAAGCAATGGTTCTTTTATCATTTCTAAAACAACT
CGGGAGGATGCAGGAAAAATATCGCTGTGCAGCTAGGAATAAGTTGGCTATATTGAGAAATTAGTCAT
ATTAGAAATTGGCCAGAAGCCAGTTATTCTTACCTATGCACCAGGGACAGTAAAAGGCATCAGTGGAG
ATCTCTATCACTGCATTGTGTGTCTGATGGAATCCCTAAGCCAPAATATCAAATGGACTATGCCAAGT
GGTTATGTAGTAGACAGGCCTCAAATTAATGGGAAATACATATTGCATGACAATGGCACCTTAGTCAT
TAAAGAAGCAACAGCTTATGACAGAGGAAACTATATCTGTAAGGCTCAAAATAGTGTTGGTCATACAC
TGATTACTGTTCCAGTAATGATTGTAGCCTACCCTCCCCGAATTACAAATCGTCCACCCAGGAGTATT
GTCACCAGGACAGGGGCAGCCTTTCAGCTCCACTGTGTGGCCTTGGGAGTTCCCAAGCCAGAAATCAC
GTGGGAGATGCCTGACCACTCCCTTCTCTCAACGGCAAGTAAAGAGAGGACACATGGAAGTGAGCAGC
TTCACTTACAAGGTACCCTAGTCATTCAGAATCCCCAAACCTCCGATTCTGGGATATACAAATGCACA
GCAAAGAACCCACTTGGTAGTGATTATGCAGCAACGTATATTCAAGTAATCCTCGAGGGC

| | | | |
|---|---|---|---|
| NOV29n, 248586774 | SEQ ID NO:692 | 473aa | MW at 52168.4 kD |
| Protein Sequence | | | |

TLEGDKLLLNCSATGEPKPQIMWRLPSKAVVDQQHRYTFHANGSLTINKVKLLDSGEYVCVARNPSGD
DTKMYKLDVVVSKPPLINGLYTNRTVIKATAVPASKAFDCFAAGTPSPEVMWIMPDNIFLTAPYYGSR
ITVHKNGTLEIRVRLSDSADFICVAR1AIEGGESVLVVQLEVLEMLRKPTFRNPFNEIVAQLGKSTAL
INCSVDGNPPPEIIWILPNGTRFSNGPQSYQYLIASNGSFIISKTTREDGKYRCAARNKVGYIEKLVI
LEIGQKPVILTYAPGTVKGISGESLSLHCVSDGIPKPNIKWTMPSGYVVDRPQINGKYILHDNGTLVI
KEATAYDRGNYICKAQNSVGHTLITVPVMIVAYPPRITNRPPRSIVTRTGAAFQLHCVALGVPKPEIT
WEMPDHSLLSTASKERTHGSEQLHLQGTLVIQNPQTSDSGIYKCTAKNPLGSDYAATYIQVILEG

| | | |
|---|---|---|
| NOV29o, 248586793 | SEQ ID NO:693 | 1420 bP |
| DNA Sequence | ORF Start: at 2 | ORF Stop: end of Sequence |

CACCCTCGAGGGGGACAAATTACTACTGAACTGCTCAGCCACTGGGGAGCCCAAACCCCAAATAATGT
GGAGGTTACCATCCAAGGCTGTGGTCGACCAGCAGCATAGGTACACATTTCATGCCAATGGGTCGTTG
ACCATCAACAAAGTGAAACTGCTCGATTCTGGAGAGTACGTATGTGTAGCCCGAAATCCCAGTGGGGA
TGACACCAAAATGTACAACTGGATGTGGTCTCTAACCTCCATTAATCAATGGTCTGTATACCAAAACA
GAACTGTTATTAAAGCCACAGCTATGAGACATTCCAPAAACACTTTGACTGCAGAGCTGAAGGGAACA
CCATCTCCTGAAGTCATGTGGATCATGCCAGACAATATTTTCCTCACAGCCCCATACTATGGAAGCAG
AATCACAGTCCATAAAAATGGAACCTTGGAAATTAGGAATGTGAGGCTTTCAGATTCAGCCGACTTTA
TCTGTGTGGCCCGATGAAGGTGGAGAGAGCGTGTTGGTAGTACAGTTAGAAGATACTGGAAAATGCTG
AGAAGACCGACATTTAGAAATCCATTTAATGAAAAAATAGTTGCCCAGCTGGGAAAGTCCACAGCATT
GAATTGCTCTGTTGATGGTAACCCACCACCGGAAATAATCTGGATTTTACCAAATGGCACACGATTTT
CCAATGGACCACAAAGTTATCAGTATCTGATAGCAAGCAATGGTTCTTTTATCATTTCTAAAACAACT
CGGGAGGATGCAGGAAATATCGCTGTGCAGCTAGGAATAAAGTTGGCTATATTGAAAGAATTAGTCAT
ATTAGAAATTGGCCAGAAGCCAGTTATTCTTACCTATGCACCAGGGACAGTAAGGCATCAAGTGGAAG
AATCTCTATCACTGCATTGTGTGTCTGATGGAATCCCTAAGCCAAATATCAAATGGACTATGCCAAGT
GGTTATGTAGTAGACAGGCCTCAAATTAATGGGAAATACATATTGCATGACATGGCACCTTAGTCAAT
TAAAGAAGCAACAGCTTATGACAGAGGAAACTATATCTGTAAGGCTCAAAATAGTGTTGGTCATACAC
TGATTACTGTTCCAGTAATGATTGTAGCCTACCCTCCCCGAATTACAAATCGTCCACCCAGGAGTATT
GTCACCAGGACAGGGGCAGCCTTTCAGCTCCACTGTGTGGCCTTGGGAGTTCCCAGCCAGAAATCAAC
GTGGGAGATGCCTGACCACTGGGTTCTCTCAACGGCAAGTAAAGAGAGGACACATGGAAGTGAGCAGC
TTCACTTACAAGGTACCCTAGTCATTCAGAATCCCCAAACCTCCGATTCTAGATAATACAAATGCACA
GCAAAGAACCCACTTGGTAGTGATTATGCAGCAACGTATATTCAAGTAATCCTCGAGGGC

| | | | |
|---|---|---|---|
| NOV29o, 248586793 | SEQ ID NO:694 | 473 aa | MW at 52200.5 kD |
| Protein Sequence | | | |

TLEGDKLLLNCSATGEPKPQIMWRTLPSKAVVDQQHRYTFHANGSLTINKVKLLDSGEYVCVARPSGD
DTKMYKLDVVSKPPLINGLYTNRTVIKATAMRHSKKIFDCRAEGTPSPEVMWIMPDNIFLTAPYYGSR
ITVHNGTLEIRNKTRLSDSADFICVARNEGGESVLVQLEVLEMLRRPTFRIAPFNEKIVAQLGKSTAL
NCSVDGNPPPEIIWILPNGTRFSNGPQSYQYLIASNGSFIISKTTREDAGKYRCAARNKVGYIEKLVI
LEIGQKPVILTYAPGTVKGISGESLSLHCVSDGIPKPNIKWTMPSGYVVDRPQINGKYILHDNGTLVI
KEATAYDRGNYICKAQNSVGHTLITVPVMIVAYPPRITNRPPRSIVTRTGAAFQLHCVALGVPKPEIT
WEMPDHSLLSTASKERTHGSEQLHLQGTLVIQNPQTSDSGIYKCTAKNPLGSDYAATYIQVILEG

| | | |
|---|---|---|
| NOV29p, 248586820 | SEQ ID NO:695 | 1420 bP |
| DNA Sequence | ORF Start: at 2 | ORF Stop: end of Sequence |

CACCCTCGAGGGGGACAAATTACTACTGAACTGCTCAGCCACTGGGGAGCCCAAACCCCAAATAATGT
ACCATCAACAAAGTGAAACTGCTCGATTCTGGAGAGTACGTATGTGTAGCCCGAAATCCCAGTGGGGA
GAACTGTTATTAAAGCCACAGCTGTGAGACATTCCAAAAAACACTTTGACTGCAGAGCTGAAGGGACA
CCATCTCCTGAGTCATGTGGATCATGCCAGACATATTTTCCTCACAGCCCCAATAACTATGGAAGCAG
AATCACAGTCCATATATGGAACCTTGGAATTAGGAATGTGAGGCTTTCAGAATTCAAGCCGAACTTTA
TCTGTGTGGCCCGAAATGAAGGTGGAGAGAGCGTGTTGGTAGTACAGTTAGAAGTACTGGAAATGCTG
AGAAGACCGGCATTTAGAAATCCATTTAATGAAAAAATAGTTGCCCAGCTGGGAAAGTCCACAGCATT
GAATTGCTCTGTTGATGGTAACCCACCACCGGAAATAATCTGGATTTTACCAAATGGCACACGATTTT
CGGGAGGATGCAGGAAAATATCGCTGTGCAGCTAGGAATAAAGTTGGCTATATTGAGAAATTAGTCAT
ATTAGAAATTGGCCAGAAGCCAGTTATTCTTACCTATGCACCAGGGACAGTAAAAGGCATCAGTGGAG
AATCTCTATCACTGCATTGTGTGTCTGATGGAATCCCTAAGCCAAATATCAAATGGACTATGCCAAGT
TAAAGAAGCAACAGCTTATGACAGAGGAAACTATATCTGTAAGGCTCAAAATAGTGTTGGTCATACAC
TGATTACTGTTCCAGTAATGATTGTAGCCTACCCTCCCCGATTACAATCGTCCACCCAGGAGTAAATT
GTCACCAGGACAGGGGCAGCCTTTCAGCTCCACTGTGTGGCCTTGGGAGTTCCCAAGCCAGAAATCAC
GTGGGAGATGCCTGACCACTCCCTTCTCTCAACGGCAAGTAAAGAGAGGACACATGGAAGTGAGCAGC
TTCACTTACAAGGTACCCTAGTCATTCAGAATCCCCAAACCTCCGATTCTGGGATATACAAATGCACA

| | | | |
|---|---|---|---|
| NOV29p, 248586820 | SEQ ID NO:696 | 473 aa | MW at 52169.4 kD |
| Protein Sequence | | | |

TLEGDKLLLNCSATGEPKPQIMWRLQSKAVVDQQHRYTFHANGSLTINKVKLLDSGEYVCVARNPSGD
DTKMYKLDVVSKPPLINGLYTNRTVIKATAVAATSKKHFDCPAGTPSPEVMWIMPDNIFLTAPYYGSR
ITVHKNGTLEIRNVRLSDSADFICVARIAIEGGESVLWQLEVLEMLRRPFRNPFNEKIVAQLGKSTAL
NCSVDGNPPPEIIWILPNGTRFSNGPQSYQYLIASNGSFIISKTTREDAGKYRCAARNKVGYIEKLVI
LEIGQKPVILTYAPGTVKGISGESLSLHCVSDGIPKPNIKWTMPSGYVVDRPQINGKYILHDNGTLVI
KEATAYDRGNYICKAQNSVGHTLITVPVMIVAYPPRITNRPPRSIVTRTGAAFQLHCVALGVPKPEIT
WEMPDHSLLSTASKERTHGSEQLHLQGTLVIQNPQTSDSGIYKCTAKNPLGSDYAATYIQVILEG

| | | |
|---|---|---|
| NOV29q, 248586824 | SEQ ID NO:697 | 1420 bP |
| DNA Sequence | ORF Start: at 2 | ORF Stop: end of Sequence |

CACCCTCGAGGGGGACAATTACTACTGAACTGCTCAGCCACTGGGGAGCCCAAAAACACCAATAATGT
GGAGGTTACCATCCAAGGCTGTGGTCGACCAGCAGCATAGGTACACATTTCATGCCAATAAGTCTTTG
ACCATCAACAAAGTGAAACTGCTCGATTCTGGAGAGTACGTATGTGTAGCCCGAATCCCAAGTGGGGA
GAACTGTTATTAAAGCCACAGCTGTGAAGACAAATTCCAACAACTTTGAAACTGCAGAAATGAAGACA
CCATCTCCTGAAGTCATGTGGATCAATGCCAGACAATATTTTCCTCACAGCCCCATACTATGGAGCAG
AATCACAGTCCATAAAAAAATGGAACCTTGGATTAGGAATGTGAGGCTTTCAGATTCAGCCGACTTTA
AGAAGACCGACATTTAGAAAATCCAATTTAATGAAAAAATAGTTGCCCAGCTGGGAGTCCACAGCATT
GATTGCTCTGTTGATGGTAACCCACCAAACCTGAAAAATCTGGATTTTACCAAATGGCACACGATTTT
CCAATGGACCACAAAGTTATCAGTATCTGATAGCAAGCAATGGTTCTTTTATCATTTCTAAACAAACT
CGGGAGGATGCAGGAATATCGCTGTGCAGCTAGGAATAAGTTGGCTAAATAAATTGAGATTAAGTAAT
ATTAGAAATTGGCCAGAAGCCAGTTATTCTTACCTATGCACCAGGGACAAGTAAAGGCATCAGTGGAG
GGTTATGTAGTAGACAGGCCTCAATTAATGGGAATACATATTGACATGACAAATGGCACCTTAGTCAT
TAAAGAAGCAACAGCTTATGACAGAGGAAACTATATATGTAAGGCTCAAAATAGTGTTGGTCATACAC
TGATTACTGTTCCAGTAATGATTGTAGCCTACCCTCCCCGAAATTACAATCGTCCACCCAGGAGTATT
GTCACCAGGACAGGGGCAGCCTTTCAGCTCCACTGTGTGGCCTTGGGAGTTCCCAAAGCCAGAATCAC
GTGGGAGATGTCTGACCACTCCCTTCTCTCAACGGCAAGTAAAGAGAGGACACATGGAAGTGAGCAGC
GCAAAGAACCCACTTGGTAGTGATTATGCAGCAACGTATATTCAAGTAATCCTCGAGGGC

| | | | |
|---|---|---|---|
| NOV29q, 248586824 | SEQ ID NO:698 | 473 aa | MW at 52198.4 kD |
| Protein Sequence | | | |

TLEGDKLLLNCSATGEPKHQIMWRLPSKAVVDQQHRYTFHANGSLTINKVKLLDSGEYVCVARNPSGD
ITVHKNGTLEIRNVRLSDSADFICVARNEGGESVLVVQLEVLEMLRRPTFRNPFNEKIVAQLGKSTAL
INCSVDGNPPPEIIWILPNGTRFSNGPQSYQYLISNGSFIISKTTREDAGKYRCAARNKVGYIEKLVI
LEIGQKPVILTYAPGTVKGISGESLSLHCVSDGIPKPNIKWTMPSGYVVDRPQINGKYILHDNGTLVI
KEATAYDRGNYICKAQNSVGHTLITVPVMIVAYPPRITNRPPRSIVTRTGAAFQLHCVALGVPKPEIT
WEMPDHSLLSTASKERTHGSEQLHLQGTLVIQNPQTSDSGIYKCTAKNPLGSDYAATYIQVILEG

| | | |
|---|---|---|
| NOV29r, 247679817 | SEQ ID NO:699 | 635 bP |
| DNA Sequence | ORF Start: at 3 | ORF Stop: end of Sequence |

AGCTCCGCGGCCGCCCCCTTCACCCTCGAGTGTCCTCGCCGCTGTGCCCTGTTATATGCACCCTCGAG
TGTCCTCGCCGCTGTGCCTGTTATATGCCTACGGAGGTACACTGCACATTTCGGTACCTGACTTCCAT
CCCAGACAGCATCCCGCCCAATGTGGAACGCATCAATTTAGGATACAACAGCTTGGTTAGATTGATGG
CCTGACAAGACCTTCTCAGATTTGCAAAGGCCTTGCAAGGTCTTAAATGAAGCTATAACAAAGTCAAA
ACTTCAGAAAGATACTTTTTATGGCCTCAGGAGCTTGACACGATTGCACATGGACCACAACAATATTG
AGTTTATACCCAAGAAAGGTTTTTGAAAAGGGCTCAACTTTCTCCGCCTGGTGCACTTGGAGGATCAG
CTCACTAAGCTCCACCCAGATACAAATTTGTCTCTTTGAGCTACCTCCAGATATTTAATCTCTTTCAT
TAAGTTCCTATACTTGTCTGATAACTTCCTGACCTCCCTCCCTCAAGAGATGGTCTCCTATATGCCTC
TCGAGGGCAAGGGTGGGCGCGCC

| | | | |
|---|---|---|---|
| NOV29r, 247679817 | SEQ ID NO:700 | 211 aa | MW at 24064.7 kD |
| Protein Sequence | | | |

APRPPPSPSSVLAAVPVICTLEPPACACYMPTEKAACTFRYLTSIPDSIPPNVERINLGYNSLVRLME
TDFSGLTKLELLMLHSNGIHTIPDKTFSDLQALQVLKMSYNKVRKLQKDTFYGLRSLTRLHMDHNNIE
FINPEVFDGLNFLRLVHLEGNQLTKLHPDTFVSLSYLQIFKISFIKFLYLSDNFLTSLPQEMVSYMPL
EGKGGRA

| | | |
|---|---|---|
| NOV29s, 248210264 | SEQ ID NO:701 | 646 bP |
| DNA Sequence | ORF Start: at 2 | ORF Stop: end of Sequence |

AACCCTCGAGATGAAGGTAAAAGGCAGAGGAATCACCTGCTTGCTGGTCTCCTTTGCTGTGATCTGCC
TGGTCGCCACCCCTGGGGGCAAGGCCTGTCCTCGCCGCTGTGCCTGTTATATGCCTACGGAGGTACAC
TGCACATTTCGGTACCTGACTTCCATCCCAGACAGCATCCCGCCCA1ATGTGGAACGCATCATTTAGG
ATACAACAGCTTGGTTAGATTGATGGAAACAGATTTTTCTGGCCTGACCAAACTGGAGTTACTCATGC
TTCACAGCAATGGCATTCACACAATCCCTGACAAGACCTTCTCAGATTTGCAGGCCTTGcAGGTCTTA
AAAATGAGCTATAACAAAGTCCGAAAACTTCAGAAAGATACTTTTTATGGCCTCAGGAGCTTGACACG
ATTGCACATGGACCACAACAATATTGAGTTTATAAACCCAGAGGTTTTTTATGGGCTCAACTTTCTCC
GCCTGGTGCACTTGGAAGGAAATCAGCTCACTAAGCTCCACCCAGATACATTTGTCTCTTTGAGCTAC
CTCCAGATATTTAAAATCTCTTTCATTAAGTTCCTATACTTGTCTGATAACTTCCTGACCTCCCTCCC
TCAAGAGATGGTCTCCTATATGCCTCTCGAGGGC

| | | | |
|---|---|---|---|
| NOV29s, 248210264 | SEQ ID NO:702 | 215 aa | MW at 24618.6 kD |
| Protein Sequence | | | |

ATLENKVKGRGITCLLVSFAVICLVATPGGKACPRRCACYMPEVHCTFRYLTSIPDSIPPNVERINLG
YNSLVRLMETDFSGLTKLELLMLHSNGIHTIPDKTFSDLQALQVLKMSYNKVRKLQKDTFYGLRSLTR
LHMDHNNIEFINPEVFYGLNFLRLVHLEGNQLTKLHPDTFVSLSYLQTFKISFIKFLYLSDNFLTSLP
QEMVSYMPLEG

| | | |
|---|---|---|
| NOV29t, 248210551 | SEQ ID NO:703 | 646 bP |
| DNA Sequence | ORF Start: at 2 | ORF Stop: end of Sequence |

CACCCTCGAGATGAAGGTAAAAGGCAGAGGAATCACCTGCTTGCTGGTCTCCTTTGCTGTGATCTGCC
TGGTCGCCACCCCTGGGGGCAAGGCCTGTCCTCGCCGCTGTGCCTGTTATATGCCTACGGAGGTACAC
TGCACATTTCGGTACCTGACTTCCATCCCAGACAGCATCCCGCCCAATGTGGAACGCATCAATTTAGG
ATACAACAGCTTGGTTAGATTGATGGAAACAGATTTTTCTGGCCTGACCAAACTGGAGTTACTCATGC
TTCACAGCAATGGCATTCACACAATCCCTGACAAGACCTTCTCAGATTTGCAGGCCTTGCAGGTCTTA
AATGAGCTATAAACAAAAGTCCGAAACTTCAGAAAAGATACTTTTTATGGCCTCAGGAGCTTGACACG
ATTGCACATGGACCACAACAATATTGAGTTTATAAACCCAGAGGTTTTTTATGGGCTCAACTTTCTCC
GCCTGGTGCACTTGGAAGGAAATCAGCTCACTAAGCTCCACCCAGATACATTTGTCTCTTTGAGCTAC
CTCCAGATATTTAAAATCTCTTTCATTAAGTTCCTATACTTGTCTGATAACTTCCTGACCTCCCTCCC
TCAAGAGATGGTCTCCTATATGCCTCTCGAGGGC

| | | | |
|---|---|---|---|
| NOV29t, 248210551 | SEQ ID NO:704 | 215 aa | MW at 24618.6 kD |
| Protein Sequence | | | |

TLEMKVKGRGITCLLVSFAVICLVATPGGKACPRRCACYMPTEVHCTFRYLTSIPDSIPPNVERINLG
YNSLVRLMETDFSGLTKLELLMLHSNGIHTIPDKTFSDLQALQVLKMSYNKVRKLQKDTFYGLRSLTR
LHMDHNNEFINPEVFYGLIAFLRLVHLEGNQLTKLHPDTFVSLSYLQIFKISFIKFLYLSDNFLTSLP
QEMVSYMPLEG

| | | |
|---|---|---|
| NOV29u, 248210824 | SEQ ID NO:705 | 646 bP |
| DNA Sequence | ORF Start: at 2 | ORF Stop: end of Sequence |

CACCCTCGAGATGAAGGTAAAAGGCAGAGGAATCACCTGCTTGCTGGTCTCCTTTGCTGTGATCTGCC
TGGTCGCCACCCCTGGGGGCAAGGCCTGTCCTCGCCGCTGTGCCTGTTATATGCCTACGGAGGTACAC
TGCACATTTCGGTACCTGACTTCCATCCCAGACAGCATCCCGCCCAATGTGGAACGCATCAATTTAGG
TTCACAGCAATGGCATTCACACAATCCCTGACAAGACCTTCTCAGATTTGCAGGCCTTGCAGGTCTTA
AAAATGAGCTATAACAAAGTCCGAAAACTTCAGAAAGATACTTTTTATGGCCTCAGGAGCTTGACACG
ATTGCACATGGACCACAACAATATTGAGTTTATAAACCCAGAGGTTTTTTATGGGCTCAACTTTCTCC
GCCTGGTGCACTTGGAAGGAAATCAGCTCACTAAGCTCCACCCAGATACATTTGTCTCTTTGAGCTAC
CTCCAGATATTTAATCTCTTTCATTAGTTCCTATACTTGTCTGAT1AAACTTCCTGACCTCCCTCCC
TCAAGAGATGGTCTCCTATATGCCTCTCGAGGGC

| | | | |
|---|---|---|---|
| NOV29u, 248210824 | SEQ ID NO:706 | 215 aa | MW at 24618.6 kD |
| Protein Sequence | | | |

TLEMKVKGRGITCLLVSFAVICLVATPGGKACPRRCACYMPTEVHCTFRYLTSIPDSIPPNVERINLG
YNSLVRLMETDFSGLTKLELLMLHSNGIHTIPDKTFSDLQALQVLKMSYNKVRKLQKDTFYGLRSLTR
LHMDHNNIEFINPEVFYGLNFLRLVHLEGNQLTKLHPDTFVSLSYLQIFKISFIKFLYLSDNFLTSLP
QEMVSYMPLEG

| | | |
|---|---|---|
| NOV29v, 248679541 | SEQ ID NO:707 | 646 bP |
| DNA Sequence | ORF Start: at 2 | ORF Stop: end of Sequence |

CACCCTCGAGATGAAGGTAAAAGGCAGAGGAATCACCTGCTTGCTGGTCTCCTTTGCTGTGATCTGCC
TGGTCGCCACCCCTGGGGGCAAGGCCTGTCCTCGCCGCTGTGCCTGTTATATGCCTACGGAGGTACAC
TGCACATTTCGGTACCTGACTTCCATCCCAGACAGCATCCCGCCCAATGTGGAACGCATCAATTTAGG
ATACAACAGCTTGGTTAGATTGATGGAAACAGATTTTTCTGGCCTGACCAAACTGGAGTTACTCATGC
TTCACAGCAATGGCATTCACACAATCCCTGACAAGACCTTCTCAGATTTGCAGGCCTTGCAGGTCTTA
AAAATGAGCTATAACAAAGTCCGAAAACTTCAGAAAGATACTTTTTATGGCCTCAGGAGCTTGACACG
ATTGCACATGGACCACAACAATATTGAGTTTATAAACCCAGAGGTTTTTTATGGGCTCAACTTTCTCC
GCCTGGTGCACTTGGAAGGAAATCAGCTCACTAAGCTCCACCCAGATACATTTGTCTCTTTGAGCTAC
CTCCAGATATTTAAAATCTCTTTCATTAAGTTCCTATACTTGTCTGATAACTTCCTGACCTCCCTCCC
TCAAGAGATGGTCTCCTATATGCCTCTCGAGGA

| | | | |
|---|---|---|---|
| NOV29v, 248679541 | SEQ ID NO:708 | 215 aa | MW at 24618.6 kD |
| Protein Sequence | | | |

TLEMKVKGRGITCLLVSFAVICLVATPGGKACPRRCACYMPTEVHCTFRYLTSIPDSIPPNVERINLG
YNSLVRLMETDFSGLTKLELLMLHSNGIHTIPDKTFSDLQALQVLKMSYNKVRKLQKDTFYGLRSLTR
LHMDHNNIEFINPEVFYGLNFLRLVHLEGNQLTKLHPDTFVSLSYLQIFKISFIKFLYLSDNFLTSLP
QEMVSYMPLEG

| | | |
|---|---|---|
| NOV29w, 247679454 | SEQ ID NO:709 | 2011 bP |
| DNA Sequence | ORF Start: at 2 | ORF Stop: end of Sequence |

AGGCTCCGCGGCCGCCCCCTTCACCCTCGAGGGGGACAAATTACTACTGAACTGCTCAGCCACTGGGG
AGCCCAAACCCCAAATAATGTGGAGGTTACCATCCAAGGCTGTGGTCGACCAGCAGCATAGAGTGGGC
AGCTGGATCCACGTCTACCCTAATGGATCCCTGTTTATTGGATCAGTAACAGAAAAAGACAGTGGTGT
CTACTTGTGTGTGGCAAGAAACAAAATGGGGGATGATCTGATACTGATGCATGTTAGCCTAGAACTGA
AACCTGCCAAAATTGACCACAAGCAGTATTTTAGAAAGCAAGTGCTCCATGGGAAAGATTTCCAAGTA
GATTGCAAGCTTCCGGCTCCCCAGTGCCAGAGATATCTTGGAGTTTGCCTGATGGAACCATGATCAAA
TATACTTCAACAAAGTTGGGGTAGCGGAGGAAGGAGATTATACTTGCTATGCCCAGAACACCCTAGGG
AAAGATGAAATGAAGGTCCACTTAACAGTTATAACAGCTGCTCCCCGGATAAGGCAGAGTAACAAAAC
AATGGGTCTTTGACCATCAACAAAGTGAAACTGCTCGATTCTGGAGAGTACGTATGTGTAGCCCGAAA
TGTATACAAATAGAACTGTTATTAAAGCCACAGCTGTGAGACATTCCAAAAAACACTTTGACTGCAGA
GCTGAAGGGACACCATCTCCTGAAGTCATGTGGATCATGCCAGACAATATTTTCCTCACAGCCCCATA
CTATGGAAGCAGAATCACAGTCCATAAAAATGGAACCTTGGAAATTAGGTATGTGAGGCTTTCAGATT
GTCCACAGCATTGAATTGCTCTGTTGATGGTAACCCACCACCTGAAATAATCTGGATTTTACCAAATG
GCACACGATTTTCCAATGGAACCACAAGTTATCAGTATCTGAATAGCAGCAATGGTTCTTTTATCATT
TCTAAAACAACTCGGGAGGATGCAGGAAAATATCGCTGTGCAGCTAGGAATAAAGTTGGCTATATTGA
GAAATTAGTCATATTAGAAATTGGCCAGAAGCCAGTTATTCTTACCTATGCAACCAAGGGACAGTAAG
CATCAGTGGAGAATCTCTATCACTGCATTGTGTGTCTGATGGAATCCCTAAAGCCAAATATCAAATGG
AACTATGCCAAGTGGTTATGTAGTAGACAGGCCTCAAATTAATGGGAATACATATTGCATGACAATGG
ACCTTAGTCATTAAAGAAGCAACAGCTTATGACAGAGGAAACTATAATCTGTAAGGCTCAAAATAGTG
TGGTCATACACTGATTACTGTTCCAGTAATGATTGTAGCCTACCCTCCCCGAATTACAAAATCGTCCA
CCCAGGAGTATTGTCACCAGGACAGGGGCAGCCTTTCAGCTCCACTGTGTGGCCTTGGGAGTTCCCAA
GCCAGAAATCACGTGGGAGATGCCTGACCACTCCCTTCTCTCAACGGCAAGTAAAGAGAGGACACATG
GAAGTGAGCAGCTTCACTTACAAGGTACCCTAGTCATTCAGAATCCCCAAACCTCCGATTCTGGGATA
TACAAATGCACAGCACTCGAGGGCAAGGGTGGGCGCGCC

| | | | |
|---|---|---|---|
| NOV29w, 247679454 | SEQ ID NO:710 | 670 aa | MW at 74046.2 kD |
| Protein Sequence | | | |

GSAAAPFTLEGDKLLLNCSATGEPKPQIMWRLPSKAVVDQQHRVGSWIHVYPNGSLFIGSVTEKDSGV
NAMQADDSGHRTRRYTLFNNGTLYFNKVGVAEEGDYTCYAQNTLGKDENKVHLTVITAAPRIRQSNKT
NKRIKAGDTAVLDCEVTGDPKPKIFWLLPSNDMISFSIDRYTFHANGSLTINKVKLLDSGEYVCVARN
PSGDDTKMYKLDVVSKPPLINGLYTNRTVIKATAVRHSKKHFDCRAEGTPSPEVMWIMPDNIFLTAPY
YGSRITVHKNGTLEIRYVRLSDSADFICVARNEGGESVLVVQLEVLEMLRRPTFRNPFNEKIVAQLGK
STALNCSVDGNPPPEIIWILPNGTRFSNGPQSYQYLIASNGSFIISKTTREDAGKYRCAARNKVGYIE
IKLVILEIGQKPVILTYAPGTVKGISGESLSLHCVSDGIPKPNKWTMPSGYVVDRPQINGKYILHDNG
TLVIKEATAYDRGNYICKAQNSVGHTLITVPVMIVAYPPRITNRPPRSIVTRTGAAFQLHCVALGVPK
PEITWEMPDHSLLSTASKERTHGSEQLHLQGTLVIQNPQTSDSGIYKCTMAEGKGGRA

| | | |
|---|---|---|
| NOV29x, 314361407 | SEQ ID NO:711 | 2902 bP |
| DNA Sequence | ORF Start: at 2 | ORF Stop: end of Sequence |

CACCCTCGAGAACTCAGATGCCTTTCTTCCCTGTGAAGCTGTTGGAAATCCCCTGCCCACCATTCATT
GGACCAGAGTCCCATCAGGACTTGATTTATCTAAAGAGGAACAGAATAGCAGGGTCCAGGTTCTCCCC
AATGGTACCCTGTCCATCCAGAGGGTGGAAATTCAGGACCGCGGACAGTACTTGTGTTCCGCATCCAA
TCTGTTTGGCACAGACCACCTTCATGTCACCTTGTCTGTGGTTTCCTATCCTCCCAGGATCCTGGAGA
GACGTAACCAAGAGATCACAGTTCATTCCGGAAGCACTGTGGAACTGAAGTGCAGAGCAGAAGGTAGG
CCAAGCCCTACAGTTACCTGGATTCTTGCAAACCAAACAGTTGTCTCAGAATCATCCCAGGGAAGTAG
GCAGGCTGTGGTGACGGTTGACGGAACATTGGTCCTCCACAATCTCAGTATTTATGACCGTGGCTTTT
ACAAATGTGTGGCCAGCAACCCAGGTGGCCAGGATTCACTGCTGGTTAAAATACAAGTCATTGCAGCA
CCACCTGTTAATTCTAAAGAGCAAGGAGGCAAGTCATTGTAGGCACTTGGGGTGAAAGTTTAACTGCC
CTGTACTGCAAAAGGAACTCCTCAGCCCAGCGTTTACTGGGTCCTCTCTGATGGCACTGAAGTGAAAC
CATTACAGTTTACCAATTCCAAAGTTGTTCTTATTTTCAAATGGGACTTTGTATATAAGAACCTAGCC
TCTTCAGACAGGGGCACTTATGAATGCATTGCTACCAGTTCCACTGGTTCGGAGCGAGAGTAGTAAAT
ATTTTGGGGACAATTACTACTGAAACTGCTCAAGCCACTGGGGAGCCCAAACCCCAAATATGTGGAGG
TTACCATCCAAGGCTGTGGTCGACCAGCAGCATAGAGTGGGCAGCTGGATCCACGTCTAACCCTATGG
TGGGGGATGATCTGATACTGATGCATGTTAAGCCTAAGACTGACCTGCCAAATTGAACCAAACAGCAG
TATTTTAGAAAGCAAGTGCTCCATGGGAAAGAATTTCCAAGTAGATTGCAAGCTTCCGGCTCCCCAGT
GCCACAGGACTAGGAGATATACCCTTTTCAACAATGGAACTTTATACTTCAACAAAGTTGGGGTAGCG
GAGGAAGGAGATTATACTTGCTATGCCCAGAACAACCCTAGGGAAGAATGATGAAAGGTCCAACTTAC
AGTTATAACAGCTGCTCCCCGGATAAGGCAGAGTAACAAAAACCAACAAAGAGAATCAGCTGGAGACA
CAGCTGTCCTTGACTGTGAGGTCACTGGGGATCCCAACCAAAATATTTTGGTTGCTGCCTTCCATAAT
GACATGATTTCCTTCTCCATTGATAGGTACACATTTCATGCCATGGGTCTTTGACCATCAAACAAAGT
GAAACTGCTCGATTCTGGAGAGTACGTATGTGTAGCCCGAAATCCCAGTGGGGATGACACCAAAATGT
ACAAACTGGATGTGGTCTCTAAACCTCCATTAATCAATGGTCTGTATACAAATAGAACTGTTATTAAA
CATGTGGATCATGCCAGACAATATTTTCCTCACAGCCCCATACTATGGAAGCAGAATCACAGTCCATA
AAAATGGAACCTTGGAAATTAGGAATGTGAGGCTTTCAGATTCAGCCGACTTTATCTGTGTGGCCCGA
AATGAAGGTGGAGAGAGCGTGTTGGTAGTACAGTTAGAAGTACTGAAAATGCTGAGAAGACCGACATT
TAGAAATCCATTTAATGAAAAATAGTTGCCCAGCTGGGAAAGTCCACAGCATTGAAATTGCTCTGTTG
AAAATATCGCTGTGCAGCTAGGAATAAAGTTGGCTATATTGAGAAATTAGTCATATTAGAAATTGGCC
AGAAGCCAGTTATTCTTACCTATGCACCAGGGACAGTAAAAGGCATCAGTGGAGATCTCTATCAACTG
CATTGTGTGTCTGATGGAATCCCTAAGCCAAATATCAAATGGACTATGCCAGTGGTTATGTAGTAAGA
CAGGCCTCAAATTAATGGGAAATACATATTGCATGACAATGGCACCTTAGTCATTAAAGAAGCAACAG
CTTATGACAGAGGAAACTATATCTGTAAGGCTCAAAATAGTGTTGGTCATACACTGATTACTGTTCCA
GTAATGATTGTAGCCTACCCTCCCCGAATTACAAATCGTCCACCCAGGAGTATTGTCACCAGGACAGG
GGCAGCCTTTCAGCTCCACTGTGTGGCCTTGGGAGTTCCCAAGCCAGAAATCACGTGGGAGATGCCTG
ACCACTCCCTTCTCTCAACGGCAAGTAAAGAGAGGACACATGGAAGTGAGCAGCTTCACTTACAAGGT
ACCCTAGTCATTCAGAATCCCCAAACCTCCGATTCTGGGATATACAAATGCACAGCAAAGAACCCACT
TGGTAGTGATTATGCAGCAACGTATATTCAAGTAATCCTCGAGGGC

| | | | |
|---|---|---|---|
| NOV29x, 314361407 | SEQ ID NO:712 | 967 aa | MW at 106911.3 kD |
| Protein Sequence | | | |

TLENSDAFLPCEAVGNPLPTIHWTRVPSGLDLSKRKQNSRVQVLPNGTLSIQRVEIQDRGQYLCSASN
QAVVTVDGTLVLHNLSIYDRGFYKCVASNPGGQDSLLVKIQVIAAPPVILEQRRQVIVGTWGESLKLP
CTAKGTPQPSVYWVLSDGTEVKPLQFTNSKLFLFSNGTLYIRNLASSDRGTYECIATSSTGSERRVVM
LTMEERVTSPRIEAASQKRTEVNFGDKLLLNCSATGEPKPQIMWRLPSKAVVDQQHRVGSWIHVYPNG
SLFIGSVTEKDSGVYLCVARNKMGDDLILMHVSLRLKPAKIDHKQYFRKQVLHGKDFQVDCKASGSPV
PEISWSLPDGTMINNAMQADDSGHRTRRYTLFNNGTLYFNKVGVAEEGDYTCYAQNTLGKDEMKVHLT
VITAAPRIRQSNKTNKRIKAGDTAVLDCEVTGDPKPKIFWLLPSNDMISFSIDRYTFHANGSLTINKV
KLLDSGEYVCVARNPSGDDTAMYKLDVVSKPPLINGLYTNRTVIKATAVRHSKKHFDCRAEGTPSPEV
MWIMPDNIFLTAPYYGSRITVHKNGTLEIRAVRLSDSADFICVARNEGGESVLVVQLEVLEMLRRPTF
RNPFNEKIVAQLGKSTALNCSVDGNPPPEIIWILPNGTRFSNGPQSYQYLIASNGSFIISKTTREDAG
KYRCAARNKVGYIEKLVILEIGQKPVILTYAPGTVKGISGESLSLHCVSDGIPKPNIKWTMPSGYVVD
RPQINGKYILHDNGTLVIKEATAYDRGNYICKAQNSVGHTLITVPVMIVAYPPRITNRPPRSIVTRTG
AAFQLHCVALGVPKPEITWEMPDHSLLSTASKERTHGSEQLHLQGTLVIQNPQTSDSGIYKCTAKAPL
GSDYAATYIQVILEG

| | | |
|---|---|---|
| NOV29y, 317803448 | SEQ ID NO:713 | 2902 bP |
| DNA Sequence | ORF Start: at 2 | ORF Stop: end of Sequence |

CACCAGATCTAACTCAGATGCCTTTCTTCCCTGTGAAGCTGTTGGAAATCCCCTGCCCACCATTCATT
GGACCAGAGTCCCATCAGGACTTGATTTATCTAAGAGGAAACAGAATAGCAGGGTCCAGGTTCTCCCC
AATGGTACCCTGTCCATCCAGAGGGTGGAAATTCAGGACCGCGGACAGTACTTGTGTTCCGCATCCAA
TCTGTTTGGCACAGACCACCTTCATGTCACCTTGTCTGTGGTTTCCTATCCTCCCAGGATCCTGGAGA
GACGTACCAAGAGATCACAGTTCATTCCGGAGCACTGTGGAAACTGAAGTGCAGAAGCAGAGGTAAGG
GCAGGCTGTGGTGACGGTTGACGGAACATTGGTCCTCCACAAATCTCAGTATTTATGACCGTGATTTT
ACAAATGTGTGGCCAGCAACCCAGGTGGCCAGGATTCACTGCTGGTTAAATACAAAGTCATTGCAGCA
TCTTCAGACAGGGGCACTTATGAATGCATTGCTACCAGTTCCACTGGTTCAAGCGAAGAGTAGTAAAT
GCTTACAATGGAAGAGCGAGTGACCAGCCCCAAGGATAGAAGCTGCAATCCCAGAGGAAACTGAGTGA
ATTTTGGGGACAAATTACTACTGAACTGCTCAGCCACTGGGGAGCCCAAACCCCAAATAATGTGGAGG
TTACCATCCAAGGCTGTGGTCGACCAGCAGCATAGAGTGGGCAGCTGGATCCACGTCTACCCTAATGG
TATTTTAGAAAGCAAAAGTGCTCCATGGGAAAGATTTCCAAGTAGATTGCAGCTTCCGGCTCCCCAGT
GAGGAAGGAAGAAATTATACTTGCTATGCCCAAAAGAACACCCTAGGGAAGATGTGAGGTCCACTTAC
AGTTATAACAGCTGCTCCCCGGATAAGGCAGAGTAACAAAACCAACAAGAGAATCAAAGCTGGAGACA
GACATGATTTCCTTCTCCATTGATAGGTACACAAAATTTCAATGCCATGGGTCTTTGACCATCACAGT
GAAACTGCTCGATTCTGGAGAGTACGTATGTGTAGCCCGAAAATCCCAGTGGGGATGACACCAAATGT
GCCACAGCTGTGAGACATTCCMACACTTTGACTGCAGAGCTGAAAAGGGAACAAACCATCTCCTGAGT
AAAATGGAACCTTGGAAATTAGGAATGTGAGGCTTTCAGATTCAGCCGACTTTATCTGTGTGGCCCGA
ATGGTAACCCACCACCTGAATAATCTGGATTTTACCATACACAACGATTTTCCAATGGAACCAAACAA
AGTTATCAGTATCTGATAGCAAAGCAAATGGTTCTTTTAATCAAAATTTCTAACACTCGGGACATGCA
AGAAGCCAGTTATTCTTACCTAATGCAACCAAGGGACAGTAAGGCATCAGTGGAGATCTCTATCACTG
CAGGCCTCAAATTAATGGGAAATACATATTGCATGACAATGGCACCTTAGTCATTAAAGAAGCAACAG
CTTAATGACAGAGGAAACTATATCTGTAAGGCTCAAATAGTGTTGGTCATACACTGATTACTGTTCCA
GTAATGATTGTAGCCTACCCTCCCCGAATTACAAATCGTCCACCCAGGAGTATTGTCACCAGGACAGG
GGCAGCCTTTCAGCTCCACTGTGTGGCCTTGGGAGTTCCCAAAGCCAGAAACACGTGGGAGATGCCTG
ACCACTCCCTTCTCTCAACGGCAAGTAAGAGAGGACACATAAGTGAGCAGCTTCACTTACGAATAAAT
ACCCTAGTCATTCAGAATCCCCAAACCTCCGATTCTGGGAATATAAATGCAAACAGCAAGAACCCACT
TGGTAGTGATTATGCAGCAACGTATATTCAAGTATCCTCGAGGGC

| | | | |
|---|---|---|---|
| NOV29y, 317803448 | SEQ ID NO:714 | 967 aa | MW at 106912.3 kD |
| Protein Sequence | | | |

TRSNSDAFLPCEAVGNPLPTIHWTRVPSGLDLSKRKQNSRVQVLPNGTLSIQRAEIQDRGQYLCSASN
LFGTDHLHTLSVVSYPPRILERRATKEITVHSGSTVELKCPGRPSPTVTVVIAAAAQTVVSESSQGSR
QAVATVDGTLVLHNLSIYDRGFYKCVASNPGGQDSLLVKIQVIAAPPVILEQRRQVIVGTWGESLKLP
CTAKGTPQPSVYWVLSDGTEVKPLQFTNSKLFLFSNGTLYIPNAASSDRGTYECIATSSTGSERRVVM
LTMEERVTSPRIEAAASQKATEVNFGDKLLLNCSATGEPKPQIMIPSAWDQQHRVGSWIHVYAAYPNG
SLFIGSVTEKDSGVYLCVARNKMGDDLILMHVSLRLKPAKIDHKQYFRKQVLHGKDFQVDCKASGSPV
PEISWSLPDGTMINNAMQADDSGHRTRRYTLFNNGTLYFNKVGVAEEGDYTCYAQNTLGKDEMKVHLT
VITAAPRTRQSNKTNKRIKAGDTAVLDCEVTGDPKPKIFWLLPSNDMISFSIDRYTFHANGSLTINKV
MWIMPDNIFLTAPYYGSRITVHKNGTLEIRNVRLSDSADFICVARNEGGESVLVVQLEVLEMLRRPTF
RNPFNEKIVAQLGKSTALNCSVDGNPPPEIIWILPNGTRFSNGPQSYQYLIASNGSFIISKTTREDAG
KYRCAARNKVGYIEKLVILEIGQKPVILTYAPGTVKGISGESLSLHCVSDGIPKPNIKWTMPSGYVVD
RPQINGKYILHDNGTLVIKEATAYDRGNYICKAQNSVVGHTLITVPVMIVAYPPRITAPPRSIVTRTG
AAFQLHCVALGVPKPEITWEMPDHSLLSTASKERTHGSEQLHLQGTLVIQNPQTSDSGIYKCTAKNPL
GSDYAATYIQVILEG

| | | |
|---|---|---|
| NOV29z, CG55776-02 | SEQ ID NO:715 | 7831 bP |
| DNA Sequence | ORF Start: ATG at 6 | ORF Stop: TGA at 7812 |

TCAGGATGAAGGTAAAAGGCAGAGGAATCACCTGCTTGCTGGTCTCCTTTGCTGTGATCTGCCTGGTC
GCCACCCCTGGGGGCAAGGCCTGTCCTCGCCGCTGTGCCTGTTATATGCCTACGGAGGTACACTGCAC
ACAGCTTGGTTAGATTGATGGAAACAGATTTTTCTGGCCTGACCAAACTGGAGTTACTCATGCTTCAC
AGCAATGGCATTCACACAATCCCTGACAAAGACCTTCTCAGATTTGCAGGCCTTGCAATGAGACTGAT
GGTCTTAAAAATGAGCTATAATAAAGTCCGAAAAACTTCAGAAGATACTTTTTATGGCCTCAGGAGCT
TGACACGATTGCACATGGACCACAACAATATTGAGTTTATAAACCCAGAGGTTTTTTATGGGCTCAAC
TTTCTCCGCCTGGTGCACTTGGAAGGAAATCAGCTCACTAAGCTCCACCCAGATACATTTGTCTCTTT
GAGCTACCTCCAGATATTTAAAATCTCTTTCATTAAGTTCCTATACTTGTCTGATAACTTCCTGACCT
CCCTCCCTCAAGAGATGGTCTCCTATATGCCTGACCTAGACAAAGCCTTTACCTGCATGGACCCATGG
ACCTGTGATTGCCATTTAAAGTGGTTGTCTGACTGGATACAGGAGAAGCCAGGTATCTATATTGTTTT
ACCAGATGTAATAATGCAAAAAGATAGAAGTCCCTCTAGTGCTCAAAGCAAGTGTCCAACTTTAATGA
ACCCTAGGACTTCTAAAGGCAAGCCGTTAGCTATGGTCTCAGCTGCAGCTTTCCAGTGTGCCAAGCCA
ACCATTGACTCATCCCTGAAATCAAAGAGCCTGACTATTCTGGAAGACAGTAGTTCTGCTTTCATCTC
TACATCGTGCTAAATACTTCATTTTCAACATTTTTGGTGTGCAACATAGATTACGGTCACATTCAGCC
AGTGTGGCAAATTTTGGCTTTGTACAGTGATTCTCCTCTGATACTAGAAAGGAGCCACTTGCTTAGTG
GAGGCAAAGATCTCAAGAAGCAGATCCCTCTTGGTTATGCAGACCTTTCCTTGCAAGCTGAAACAGAC
TGCCACCACATTCAGTACATTACAGATCCAGTACTCCAGTGATGCTCAAATCACTTTACCAAGAGCAG
GTCTTGGTAGGTGGACCGTTGGCCTGAACTGCCCAGGCCAGGAGACCCCACCCCAACACGTGGAATTG
GCTTCTAGCTGATGGAAGTAGTGAGAGCCCCTTATGTCAGTGAGGATGGACAATCCTAAATAAGAACA
AAAGTGGAAAATTGGAACTCCAGATGGCTGATAGTTTTGACACAGGCGTATATCACTGTATAAGCAGC
ATTATGATGATGCAGATATTCTCACCTATAGGATACTGTGGTAAGAAACCTTTGGTCGAGCCTAATCA
GGAAAATGGGATTCATCACACAGTTTTCATTGGTGAAACACTTGATCTTCCATGCCATTCTACTGGTA
AAAGTTCTAAACAATGGCACATTAAGAATATTACAGGTCACCCCGAAAGACCAAGGTTATTATCGCTG
TGTGGCAGCCAACCCATCAGGGGTTGATTTTTTGATTTTCCAAGTTTCAGTCA7AGATGAGGACAAAA
GGCCCTTGGAGCATGATGGAGAAACAGAGGGATCTGGACTTGATGAGTCCAATCCTATTGCTCATCTT
AAGGAGCCACCAGGTGCACAACTCCGTACATCTGCTCTGATGGAGGCTGAGGTTGGAAAACACACCTC
AAGCACAAGTAAGAGGCACAACTATCGGGAATTAACACTCCAGCGACGTGGAGATTCAACACATCGAC
GTTTTAGGGAGAATAGGAGGCATTTCCCTCCCTCTGCTAGGAGAATTGACCCACAACATTGGGCGGCA
CTGTTGGAGAAAGCTAAAAAGAATGCTATGCCAGACAAGCGAGAAAATACCACAGTGAGCCCACCCCC
AGTGGTCACCCAACTCCCAAACATACCTGGTGAAGAAGACGATTCCTCAGGCATGCTCGCTCTACATG
AGGAATTTATGGTCCCGGCCACTAAAGCTTTGAACCTTCCAGCAAGGACAGTGACTGCTGACTCCAGA
ACAATATCTGATAGTCCTATGACAAACATAAATTATGGCACAGAATTCTCTCCTGTTGTGAATTCACA
AATACTACCACCTGAAGAACCCACAGATTTCAAACTGTCTACTGCTATTAAAACTACAGCCATGTCAA
AGAATATAAACCCAACCATGTCAAGCCAAATACAAGGCACAACCAATCAACATTCATCCACTGTCTTT
AAGTAGACCCCCAATAACAGTAAGGACTATGATCAAAGATGTCAATGTCAAAATGCTTAGTAGCACCA
GAACCCAGGCACAATCACTTCTATTCTCACACTACTCAAATACTTAGCACCTCCACGTTCCCTTCAGA
TCCACACACAGCTGCTCATTCTCAGTTTCCGATCCCTAGAAATAGTACAGTTAACATCCCGCTGTTCA
GACGCTTTGGGAGGCAGAGGAAAATTGGCGGAAGGGGGCGGATTATCAGCCCATATAGAACTCCAGTT
CTGCGACGGCATAGATACAGCATTTTCAGGTCAACAACCAGAGGTTCTTCTGAAAAAAGCACTACTGC
ATTCTCAGCCACAGTGCTCAATGTGACATGTCTGTCCTGTCTTCCCAGGGAGAGGCTCACCACTGCCA
CAGCAGCATTGTCTTTTCCAAGTGCTGCTCCCATCACCTTCCCCAAAGCTGACATTGCTAGAGTCCCA
TCAGAAGAGTCTACAACTCTAGTCCAGAATCCACTATTACTACTTGAGAACAAACCCAGTGTAGAGAA
AACAACACCCACAATAAAATATTTCAGGACTGAAATTTCCCAAGTGACTCCAACTGGTGCAGTCATGA
CATATGCTCCAACATCCATACCCATGGAAAAAACTCACAAAGTAAACGCCAGTTACCCACGTGTGTCT
AGCACCAATGAAGCTAAAAGAGATTCAGTGATTACATCGTCACTTTCAGGTGCTATCACCAAGCCACC
AATGACTATTATAGCCATTACAAGGTTTTCAAGAAAGGAAATTCCCTGGCAAACAGAAACTTTGTATA
ACCATAACCCAAAGGCAGATTAAGGAATCAACATAAAGTTAAAGTTTACAAAAAGCACAGCTGTGATG
CTTCCTAAACATCTCCTGCTTTACCACAAGAGACAAAGTCTCCCCTCGCACCACACTACGACCAAAAC
ACACAATCCTGGAAGTCTTCCAACAAAGAAGGAGCTTCCCTTCCCACCCCTTAACCCTATGCTTCCTA
GTATTATAAGCAAAGACTCAAGTACAAAAAGCATCATATCAACGCAAACAGCAATACCAGCAACAACT
AAGAGAACAGGAGCCTCAAAAGAAGAACAGGACTGACCCAAACATCTCTCCAGACCAGAGTTCTGGCT
TCACTACACCCACTGCTATGACACCTCCTGTTCTAACCACAGCCGAAACTTCAGTCAAGCCCAGTGTC
AACTCTTAATCTGACAGATGTGATTGAGAACTAGCCCAAGCAAGTACTCAAGACTTTGAAGAGCACAA
CACTCAACCATACCACCATTCTTGAGCAGCAGTGCTACTCTAATGCCAGTTCCCATCTCCCCTCCCTT
TACTCAGAGAGCAGTTACTGACAACGTGGCGACTCCCATTTCCGGGCTTATGACAAATACAGTGGTCA
AGCTGCACGAATCCTCAAGGCACAATGCTACCACAGCATTAGTAAGCAGAAGGTTAAAAACATCCCCC
AAGGTTCACCCAATGCCAAGTTCACAATTGGAACCACTCACTTCATCTACTCTAAATCTGTTACATTC
TTGGCTACTACAGGCCTGTCCGAGGCCACCACTCTTGTTTCAGATTGGGAATGGAACAGAGACACAAA
GAAGAGTGACTTTGATAAGAACCAGTTCAAGAAGCAACAACTTCCAAAACTCCTTCCCTTTGACTCTT
CCCATCAGGTATGTCAGGACTTGAATTTAAATCTAAGAGGCAGAAATAGCAAGTCCAGGTTCTCCCCA
ATGGTACCCTGTCCATCCAGAGGGTGGAATTCAGGACCGCGGACAGTACTTGTGTTCCGCATCCAAAT
CTGTTTGGCACAGACCACCTTCATGTCACCTTGTCTGTGGTTTCCTATCCTCCCAGGATCCTGGAGAG
CAGGCTGTGGTGACGGTTGACGGAACATTGGTCCTCCACAATCTCAGTATTTATGACCGTGGCTTTTA
CAAATGTGTGGCCAGCAACCCAGGTGGCCAGGATTCACTGCTGGTTAAAAACAAAGTCATTGCAGCAC
TGTACTGCAAAGGAACTCCTCAAGCCCAAGCGTTTAAACTGGGTCCTCTCTGATGGCACTGAGTGACC
ATTACAGTTTACCATTCCAAGTTGTTCTTAATTTTCAAATGGGAACTTTGAATATAGAAAQCTAGCCT
CTTCAGACAGGGGCACTTATGATGCATTGCTACCAGTTCCAACTGGTTCGGAAGCGAGAGTAAGTATG
CTTACAATGGAAGAGCGAGTGACCAGCCCCAGGTAAGAAGCTGCATCCCAGAAAAGGACTGAAGTGAA
TTTTGGGGACAAATTACTACTGAACTGCTCAGCCACTGGGGAGCCCAAACCCCAAATAATGTGGAGGT
TACCATCCAAGGCTGTGGTCGACCAGCAGCATAGGGTGGGCAGCTGGATCCACGTCTACCCTTATGGA
TCCCTGTTTATTGGATCAGTAACAGAAAAAGACAGTGGTGTCTACTTGTGTGTGGCAAGAAACAAAAT
GGGGGATGATCTGATACTGATGCATGTTAGCCTAAGACTGACCTGCCAAATTGAACCAACAAGCAAGT
ATTTTAGAAAGCAAGTGCTCCATGGGAAGATTTCCAAGTAGATTGCAAAGCTTCCGGCTCCCCAAGTG
CCAGAGATATCTTGGAGTTTGCCTGATGGAACCATGATCAACAATGCAATGCAAGCCGATGACAGTGG
CCACAGGACTAGGAGATATCCCTTTTCAACAATGGACTTTATACTTCAAACAAAGTTGGGGTAAGCGG
AGGAAGGAGATTATACTTGCTATGCCCAGAACACCCTAGGGAAAGATGAAATGAAGGTCCACTTAACA
GTTATAACAGCTGCTCCCCGGATAAGGCAGAGTAACAAAACCAACAAGAGAATCAAAGCTGGAGACAC
AGCTGTCCTTGACCGTGAGGTGACTGGGGATCCCAAACCAAAAATATTTTGGTTGCTGCCTTCCAATG
GGTCTTTGACCATCAAACAAAGTGAAACTGCTCGATTCTGGAGAGTACGTATGTGTAGCCCGAATCCC
AGTGGGGATGACACCAAATGTAACAAACTGGAAATGTGGTCTCTACCTCCATTATCAAATGGTCTGTA
TACAAATAGAACTGTTATTAAAGCCACAGCTGTGAAGAACAATTCCAAACACTTTGACTGCAGAGCTG
GGAAGCAGAATCACAGTCCATAAAAATGGAACCTTGGAAATTAGGAATGTGAGGCTTTCAGATTCAGC
CGACTTTATCTGTGTGGCCCGAAATGAAGGTGGAGAGAAGCGTGTTGGTAGTACAGTTAGAGTACTGG
AAATGCTGAGAAGACCGACATTTAGAATCCATTTAATGAAATAAAGTTACCAGCTGGGATAAAAGTCC
ACAGCATTGAATTGCTCTGTTGATGGTACCCACCACCTGTAATCTGGAAATTTTAAACCAAATGGCAC
ACGATTTTCCAATGGACCACAGTTATCAGTATCTGATAGCAGCAATGGTTCTTTTAATCAATTTCTAA
AAACAACTCGGGAGGATGCAGGAATATCGCTGTGCAGCTAGGAATAAAGTTGGCTAATATTGAGAAAA
TTAGTCATATTAGAATTGGCCAGAAGCCAGTTATTCTTACCTATGCAACCAGGGACAGTAAAGGCAAT
TCATACACTGATTACTGTTCCAGTAATGATTGTAGCCTACCCTCCCCGAATTACAATCGTCCAACCCA
TGCCAAGTGGTTATGTAGTAGACAGGCCTCATTATGGGATACATAATTGCATGAAACAATAAACAACC
TTAGTCATTAAAGAAGCAACAGCTTATGACAGAGGAACTATATCTGTA3AGGCTCAAATAAGTGTTGG
TCATACACTGATTACTGTTCCAGTAATGATTGTAGCCTACCCTCCCCGAAATTACATCGTCCAACCCA
GGAGTATTGTCACCAGGACAGGGGCAGCCTTTCAGCTCCACTGTGTGGCCTTGGGAGTTCCCAAGCCA
GAAATCACATGGGAGATGCCTGACCACTCCCTTCTCTCAACGGCAAGTAAAGAGAGGACACATGGAAG
TGAGCAGCTTCACTTACAAGGTACCCTAGTCATTCAGAATCCCCAAACCTCCGATTCTGGGATATACA
AATGCACAGCAAAGAACCCACTTGGTAGTGATTATGCAGCAACGTATATTCAAGTAATGTGACATGAA
ATAATAAAGTC

| | | | |
|---|---|---|---|
| NOV29z, CG55776-02 | SEQ ID NO:716 | 2602 aa | MW at 288454.2 kD |
| Protein Sequence | | | |

MKVKGRGITCLLVSFAVICLVATPGGKACPRRCACYMPTEAHCTFRYLTSIPDSIPPNVERINLGYNS
LVRLMETDFSGLTKLELLMLHSNGIHIPDKTFSDLQALQVRLMVLKMSYNKKTRKLQKDTFYGLRSLT
RLHMDHNNIEFINPEVFYGLNFLRLVAHLEGNQLTKLHPAFVSLSYLQIFKISFIKFLYLSDNFLTSL
PQEASYMPDLDSLYLHGNPWTCDCHLKWLSDWIQEKPGIYNTAPDVIKCYAADRSPSSAQQCPLCMNP
RTSKGKPLAMKTSAAAFQCAKPTIDSSLKSKSLTILEDSSSAFSPQGFMAPFGSLTLNMTDQSGNEAN
MVCSIQKPSRTSPIAFTEENDYIVLNTSFSTFLVCNIDYGHIQPKAWQILAYSDSPLILERSHLLSET
PQLYYKYKQVAPKPEDIFTNIEADLRADPSWLMQDQISLQLNRTATTFSTLQIQYSSDAQITLPRAEM
RPVKHKWTMISRDNNTKLEHTVLVGGTVGLNCPGQGDPTPHVDWLLADGSKVRAPYVSEDGRILIDKS
GKLELQMADSFDTGVYHCISSNYDDADILTYRITVVEPLVEAYQENGIHHTVFIGETLDLPCHSTGIP
DASISWVIPGNNVLYQSSRDKKVLNNGTLRILQVTPKDQGYYRCVAANPSGVDFLIFQVSVKMKGQRP
LEHDGETEGSGLDESNPIAHLKEPPGAQLRTSALMEAEVGKAHSSTSKRHNYRELTLQRRGDSTHRRF
RENRRHFPPSARRIDPQBWAALLEKAKKNAMPDKRENTTVSPPPVVTQLPNIPGEEDDSSGMLALHEE
FMVPATKALNLPARTVTADSRTISDSPMTNINYGTEFSPVVNSQILPPEEPTDFKLSTAIKTTAMSKN
INPTMSSQIQGTTNQHSSTVFPLLLGATEFQDSDQMGRGREHFQSRPPITVRTMIKDVNVKMLSSTTN
KLLLESVNTTNSHQTSVREVSEPRHNHFYSHTTQILSTSTFPSDPHTAAHSQFPIPRNSTVNIPLFRR
FGRQRKIGGRGRIISPYRTPVLRRHRYSIFRSTTRGSSEKSTTAFSATVLNVTCLSCLPRERLTTATA
ALSFPSAAPITFPKADIARVPSEESTTLVQNPLLLLENKPSVEKTTPTIKYFRTEISQVTPTGAVMTY
APTSIPMEKTHKVNASYPRVSSTNEAKRDSVITSSLSGAITKPPMTIIAITRFSRRKIPWQQNFVNNH
NPKGRLRNQHKVSLQKSTAVMLPKTSPALPQRQSLPSHHTTTKTHNPGSLPTKKELPFPPLNPMLPSI
ISKDSSTKSIISTQTAIPATTPTFPASVITYETQTERSRAQTIQREQEPQKKNRTDPNISPDQSSGFT
TPTAMTPPVLTTAETSVKPSVSAFTHSPPENTTGISSTISFHSRTLNLTDVIEELAQASTQTLKSTIA
SETTLSSKSHQSTTTRKAIRHSTIPPFLSSSATLMPVPISPPFTQRAKTTDNVATPISGLMTNTVVKL
HESSRHNAKPQQLVAEVATSPKVHPNAKFTIGTTHFIYSNLLHSTPMPALTTVKSQNSKLTPSPWAEN
QFWHKPYSEIAEKGKKPEVSMLATTGLSEATTLVSDWDGQKNTKKSDFDKKPVQEATTSKLLPFDSLS
RYIFEKPRIVGGKAASFTIPANSDAFLPCEAVGNPLPTIHWTRVPSGMSGLDLSKRKQNSRVQVLPNG
TLSIQRVEIQDRGQYLCSASNLFGTDHLHVTLSVVSYPPRILERRTKEITVHSGSTVELKCRAEGRPS
PTVTWILANQTVVSESSQGSRQAVVTVDGTLVLHNLSIYDRGFYKCVASNPGGQDSLLVKIQVIAAPP
DRGTYECIATSSTGSERRVVMLTMEERVTSPRIEAASQKRTEVNFGDKLLLNCSATGEPKPQIMWRLP
SKAVVDQQHRVGSWIHVYPNGSLFIGSVTEKDSGVYLCVARNKMGDDLILMHVSLRLKPAKIDHKQYF
RKQVLHGKDFQVDCKASGSPVPEISWSLPDGTMINNNMQADDSGHRTRRYTLFNNGTLYFNKVGVAEE
GDYTCYAQNTLGKDEMKVHLTVITAAPRIRQSNKTNKRIKAGDTAVLDREVTGDPKPKIFWLLPSNGS
LTINKVKLLDSGEYVCVARNPSGDDTKMYKLDVVSKPPLINGLYTNRTVIKATAVRHSKKHFDCRAEG
TPSPEVMWIMPDNIFLTAPYYGSRITVHKNGTLEIRNVRLSDSADFICVARNEGGESVLVVQLEVLEM
LRRPTFRNPFNEKIVAQLGKSTALNCSVDGNPPPEIIWILPNGTRFSNGPQSYQYLIASNGSFIISKT
TREDAGKYRCAARNKVGYIEKLVILEIGQKPVILTYAPGTVKGISGESLSLHCVSDGIPKPNIKWTMP
SGYVVDRPQINGKYILHDNGTLVIKEATAYDRGNYICKAQNSVGHTLITVPVMIVAYPPRITNRPPRS
IVTRTGAAFQLHCVALGVPKPEITWEMPDHSLLSTASKERTHGSEQLHLQGTLVIQNPQTSDSGIYKC
TAKNPLGSDYAATYIQVI

| | | |
|---|---|---|
| NOV29aa, CG55776-03 | SEQ ID NO:717 | 8270 bP |
| DNA Sequence | ORF Start: ATG at 6 | ORF Stop: TGA at 7779 |

TCAGGATGAAGGTAAAAGGCAGAGGAATCACCTGCTTGCTGGTCTCCTTTGCTGTGATCTGCCTGGTC
ATTTCGGTACCTGACTTCCATCCCAGACAGCATCCCGCCCAATGTGGAACGCATCAATTTAGGGTACA
ACAGCTTGGTTAGATTGATGGAAACAGATTTTTCTGGCCTGACCAAACTGGAGTTACTCATGCTTCAC
AGCAATGGCATTCACACAATCCCTGACAAGACCTTCTCAGATTTGCAGGCCTTGCAGGTGAGACTGAT
GGTCTTAATGAGCTATAATAAAGTCCGAAAACTTCAGAAAGATAACTTTTTAATGGCCTCAAGGAGCT
TGACACGATTGCACATGGACCACAACAATATTGAGTTTATAAACCCAGAGGTTTTTTATGGGCTCAAC
TTTCTCCGCCTGGTGCACTTGGAAGGAAATCAGCTCACTAAGCTCCACCCAGATACATTTGTCTCTTT
CCCTCCCTCAAGAGATGGTCTCCTATATGCCTGACCTAGACAGCCTTTACCTGCATGGAAACCCATGG
ACCTGTGATTGCCATTTAAAGTGGTTGTCTGACTGGATACAGGAGAAGCCAGGTATCTATATTGTNTT
ACCAGATGTAATAAAATGCAAAAAAGATAGAAGTCCCTCTAGTGCTCAGCAGTGTCCACTTTGCATGA
ACCCTAGGACTTCTAAAGGCAAGCCGTTAGCTATGGTCTCAGCTGCAGCTTTCCAGTGTGCCAAGCCA
ACCATTGACTCATCCCTGAAATCAAAGAGCCTGACTATTCTGGAAGACAGTAGTTCTGCTTTCATCTC
TCCCCAAGGTTTCATGGCACCCTTTGGCTCCCTCACTTTGAATATGACAGATCAGTCTGGAAATGAAG
CTAACATGGTCTGCAGTATTCAAAAGCCCTCAAGGACATCACCCATTGCATTCACTGAAGAAAATGAC
TACATCGTGCTAAATACTTCAATTTTCAACATTTTTGGTGTGCACATAGATTACGGTCACATTCAGCC
AGTGTGGCAAATTTTGGCTTTGTACAGTGATTCTCCTCTGATACTAGAAAGGAGCCACTTGCTTAGTG
AAACACCGCAGCTCTATTACAAATATAAACAGGTGGCTCCTAAGCCTGAAGACATTTTTACCAACATA
GAGGCAGATCTCAGAGCAGATCCCTCTTGGTTAATGCAAGACCAAATTTCCTTGCAGCTGAACAGAAC
TGCCACCACATTCAGTACATTACAGATCCAGTACTCCAGTGATGCTCAAATCACTTTACCAAGAGCAG
AGATGAGGCCAGTGAAACACAAATGGACTATGATTTCAAGGGATAACAATACTAAGCTGGAACATACT
GTCTTGGTAGGTGGAACCGTTGGCCTGAACTGCCCAGGCCAAGGAGACCCCACCCCACACGTGGATTG
GCTTCTAGCTGATGGAGTAAAGTGAGAGCCCCTTATGTCAGTGAGGATGGACGGATCCTAATAGAACA
AAAGTGGAAAATTGGAACTCCAGATGGCTGATAGTTTTGACACAGGCGTATATCACTGTATAAGCAGC
AATTATGATGATGCAGATATTCTCACCTATAGGATAACTGTGGTAGAACCTTTGGTCGAAGCCTATCA
GGAAAATGGGATTCATCACACAGTTTTCATTGGTGAAACACTTGATCTTCCATGCCATTCTACTGGTA
TCCCAGATGCCTCTATTAGCTGGGTTATTCCAGGAAACAATGTGCTCTATCAGTCATCAAGAGACAAG
AAAGTTCTAAACAATGGCACATTAAGAATATTACAGGTCACCCCGAAAGACCAAGGTTATTATCGCTG
TGTGGCAGCCAACCCATCAGGGGTTGATTTTTTGATTTTCCAAGTTCAGTCAAGATGAAAGGAACAAA
GGCCCTTGGAGCATGATGGAGAAACAGAGGGATCTGGACTTGATGAGTCCAATCCTATTGCTCATCTT
AAGGAGCCACCAGGTGCACAACTCCGTACATCTGCTCTGATGGAGGCTGAAGGTTGGAAACAAACCTC
AAGCACAAGTAAGAGGCACAACTATCGGGAATTAACACTCCAGCGACGTGGAGATTCAACACATCGAC
GTTTTAGGGAGAATAGGAGGCATTTCCCTCCCTCTGCTAGGAGAATTGACCCACAACATTGGGCGGCA
CTGTTGGAGAAAGCTAAAAAGAATGCTATGCCAGACAAGCGAGAAAATACCACAGTGAGCCCACCCCC
AGTGGTCACCCAACTCCCAAACATACCTGGTGAAGAAGACGATTCCTCAGGCATGCTCGCTCTACATG
AGGATTTATGGTCCCGGCCACTAAAGCTTTGAACCTTCCAGCAAGGACAAGTGACTGCTGACTCCAGA
ACAATAATCTGATAGTCCTATGACAACATAAATTATGGCACAGAAATTCTCTCCTGTTGTGATTCACA
AATACTACCACCTGAAGAACCCACAGATTTCAAAGTCTCTACTGCTATTAAAACTACAGCCATGTCAA
AGAATATAAACCCAACCATGTCAAGCCAAATACAAGGCACAACCAATCAACATTCATCCACTGTCTTT
CCACTGCTACTTGGAGCAACTGAATTTCAGGACTCTGACCAGATGGGAAGAGGAAGAGAGCATTTCCA
AAGTAGACCCCCAATAACAGTAAGGACTATGATCAGATGTCAATGTCAAAATGCTTAAGTAAGCACCA
CCAACAAACTATTATTAGAGTCAGTAAATACCACAAATAGTCATCAGACATCTGTAAGAGAAGTGAGT
GAACCCAGGCACAATCACTTCTATTCTCACACTACTCAAATACTTAGCACCTCCACGTTCCCTTCAGA
TCCACACACAGCTGCTCATTCTCAGTTTCCGATCCCTAGAAATAGTACAGTTAACATCCCGCTGTTCA
GACGCTTTGGGAGGCAGAGGAAAATTGGCGGAAGGGGGCGGATTATCAGCCCATATAGAACTCCAGTT
CTGCGACGGCATAGATACAGCATTTTCAGGTCAACAACCAGAGGTTCTTCTGAAAAAAGCACTACTGC
ATTCTCAGCCACAGTGCTCAATGTGACATGTCTGTCCTGTCTTCCCAGGGAGAGGCTCACCACTGCCA
CAGCAGCATTGTCTTTTCCAAGTGCTGCTCCCATCACCTTCCCCAAAGCTGACATTGCTAGAGTCCCA
TCAGAAGAGTCTACAACTCTAGTCCAGAATCCACTATTACTACTTGAGAACAAACCCAGTGTAGAGAA
CATATGCTCCAACATCCATACCCATGGAAAAAACTCACAAAGTAAACGCCAGTTACCCACGTGTGTCT
AGCACCAATGAAGCTAAAAGAGATTCAGTGATTACATCGTCACTTTCAGGTGCTATCACCAAGCCACC
ACCATAACCCAAAAGGCAGATTAAAGGAAATCAACAATAAAGTTAGTTTACAAAGCACAGCTGTGATG
CTTCCTAAAACATCTCCTGCTTTACCACAGAGACAAAGTCTCCCCTCGCACCACACTACGACCAAAAC
ACACAATCCTGGAAGTCTTCCAACAAAGAAGGAGCTTCCCTTCCCACCCCTTAACCCTATGCTTCCTA
GTATTATAAGCAAGACTCAAGTAACAAGCAATCAATAATCAACGCAAACAAGCAAATACCAGCACACT
CCTACCTTCCCTGCATCTGTCATCACTTATGAAACCCAAACAGAGAGATCTAGAGCACAAACAATACA
AAGAGAACAGGAGCCTCAAAAGAAGAACAGGACTGACCCAAACATCTCTCCAGACCAGAGTTCTGGCT
TCAACTACACCCACTGCTATGACACCTCCTGTTCTAACCACAGCCGAAACTTCAGTCAGCCCAGTGTC
TCTGCATTCACTCATTCCCCACCAGAAACAACAACTGGGAATTTCAAGCACAATCAGTTTTCATTCAG
AACTCTTATCTGACAGATGTGATTGAAAGAAACTAGCCCAAGCAAGTACTCAGACTTTGAAGAGCACA
TTGCTTCTGAAACAACTTTGTCCAGCAATCACACCAGAGTACCACAACTAGGAAAGCAAATCATTAGA
CACTCAACCATACCACCATTCTTGAGCAGCAGTGCTACTCTAATGCCAGTTCCCATCTCCCCTCCCTT
TACTCAGAGAGCAGTTACTGACAACGTGGCGACTCCCATTTCCGGGCTTATGACAAATACAGTGGTCA
AGCTGCACGAATCCTCAAGGCACAATGCTACCAACAAGCAATTAGTAGCAGAGGTTGCAACATCCCCC
AAGGTTCACCCAAATGCCAAGTTCACAATTGGAACCACTCACTTCATCTACTCTAATCTGTTACATTC
TACTCCCATGCCAGCACTACAACAGTTAAATCAACAGAATTCTAAATTAACTCCATCTCCCTGGGCAG
AAAACCAATTTTGGCACAAACCATACTCAAGAAATTGCTGAAAAGGCAAAAAGCCAGAAGTAAGCATG
TTGGCTACTACAGGCCTGTCCGAGGCCACCACTCTTGTTTCAGATTGGGATGGACAGAAGAACACAAA
GAAGAGTGACTTTGATAAGAAACCAGTTCAAGAAGCAACAACTTCCAAACTCCTTCCCTTTGACTCTT
TGTCTAGGTATATATTTGAAGCCCAGGAAAATAGTTGGAAAAGAGCTGCAAGTTTTAACTATTCCAAT
AACTCAGATGCCTTTCTTCCCTGTGAAGCTGTTGGAAATCCCCTGCCCACCATTCATTGGACCAGAGT
CCCATCAGGTATGTCAGGACTTGATTTATCTAAGAGGAAACAGAATAGCAGGGTCCAGGTTCTCCCCA
ATGGTACCCTGTCCATCCAGAGGGTGGAAATTCAGGACCGCGGACAGTACTTGTGTTCCGCATCCAAT
ACGTACCAAAGAGATCACAGTTCATTCCGGAAGCACTGTGGAACTGAAGTGCAGAGCAGAAGGTAGGC
CAAGCCCTACAGTTACCTGGATTCTTGCAAACCAAACAGTTGTCTCAGAATCATCCCAGGGAAGTAGG
AGGCTGTGGTGACGGTTGACGGAACATTGGTCCTCCACAATCTCAGTATTTAATGACCGTGGCTTTTA
CAAATGTGTGGCCAGCAACCCAGGTGGCCAGGATTCACTGCTGGTTAAATAACAAGTCATTGCAGCAC
CACCTGTTATTCTAGAGCAAGGAGGCAAGTCATTGTAAGGCAACTTGGGGTGAAAGTTTAAACTGCCC
TGTACTGCAAAAGGAACTCCTCAGCCCAGCGTTTACTGGGTCCTCTCTGATGGCACTGAAGTGAAACC
ATTACAGTTTACCAATTCCAAGTTGTTCTTATTTTCAAATGGGACTTTGTATATAAGAAACCTAGCCT
CTTCAGACAGGGGCACTTATGAATGCATTGCTACCAGTTCCACTGGTTCGGAGCGAAGAGTAGTAATG
CTTACAATGGAAGAGCGAGTGACCAGCCCCAGGATAGAAAGCTGCATCCCAGAAGGACTGAAAGTGAA
TTTTGGGGACATTACTACTGAACTGCTCAGCCACTGGGGAAGCCCAAAACCCCAAATAATGTGGAGGT
TACCATCCAAGGCTGTGGTCGACCAGCAGCATAGAGTGGGCAGCTGGATCCACGTCTACCCTAATGGA
TCCCTGTTTATTGGATCAGTAACAGAAGACAAGTGGTGTCTAACTTGTGTGTGGCAAAGAAACAAAAT
GGGGGATGATCTGATACTGATGCATGTTAGCCTAGAACTGAAACCTGCCAAAATTGACCACAAGCAGT
ATTTTAGAAAGCAAGTGCTCCATGGGAAAGATTTCCAAGTAGATTGCAAAGCTTCCGGCTCCCCAGTG
CCAGAGATATCTTGGAGTTTGCCTGATGGAACCATGATCAACAATGCAAATGCAGCCGATGACAGTGG
CCACAGGACTAGGAGATATACCCTTTTCAACAATGGAACTTTATACTTCAACAAAGTTGGGGTAGCGG
AGGAAGGAGATTATACTTGCTATGCCCAGAACACCCTAGGGAAAGATGAAATGAAGGTCCACTTAACA
GTTATAACAGCTGCTCCCCGGATAAGGCAGAGTAACAAACCMACAAGAGAAATCAAAGCTGGAGACAC
AGCTGTCCTTGACTGTGAGGTCATTCATGCCAAATGGGTCTTTGAACCATCAACAAGTGAACTGCTCG
ATTCTGGAGAGTACGTATGTGTAGCCCGAAATCCCAGTGGGGAATGACACCAAATGTACAAACTGGAT
GTGGTCTCTAAACCTCCATTAATCAATGGTCTGTATACAAATAGAACTGTTATTAAAGCCACAGCTGT
GAGACATTCCAAAAAACACTTTGACTGCAGAGCTGAAGGGACACCATCTCCTGAAGTCATGTGGATCA
TGCCAGACAATATTTTCCTCACAGCCCCATACTATGGAAGCAGAATCACAGTCCATAAAAATGGAACC
TTGGAAATTAGGAATGTGAGGCTTTCAGATTCAGCCGACTTTAATCTGTGTGGCCCGAAATGAGGTGG
AGAGAGCGTGTTGGTAGTACAGTTAGAAGTACTGGAAAATGCTGAAGAAGACCGACATTTAGATCCAT
TTAATGAAAAAATAGTTGCCCAGCTGGGAAAGTCCACAGCATTGAATTGCTCTGTTGATGGTAACCCA
CCACCTGAAATAATCTGGATTTTACCAATGGCACACGATTTTCCAATGGACCACAAAAGTTATCAGTA
TCTGATAGCAAGCAATGGTTCTTTTATCATTTCTAACAACTCGGGAGGATGCAGGAAAAAATCATGCT
GTGCAGCTAGGAATAAAGTTGGCTATATTGAGAMATTAGTCATATTAGAAATTGGCCAGAAGCCAGTT
ATTCTTACCTATGCACCAGGGACAGTAAAGGCATCAGTGGAGAAACTCTAATCAACTAATTGTGTGTC
TGATGGAATCCCTAAGCCAAATATCAAATGGACTATGCCAAGTGGTTATGTAGTAGACAGGCCTCAAA
TTAATGGGAAATACATATTGCATGACAATGGCACCTTAGTCATTAAAGAAGCAACAGCTTATGACAGA
GGAAACTATATCTGTAAGGCTCAAAATAGTGTTGGTCATACACTGATTACTGTTCCAGTAATGATTGT
AGCCTACCCTCCCCGAATTACAAATCGTCCACCCAGGAGTATTGTCACCAGGACAGGGGCAGCCTTTC
AGCTCCACTGTGTGGCCTTGGGAGTTCCCAAGCCAGAAATCACGTGGGAGATGCCTGACCACTCCCTT
CTCTCAACGGCAAGTAAAGAGAGGACACATGGAAGTGAGCAGCTTCACTTACAGGTACCCTAAGTCAT
TCAGAATCCCCAAACCTCCGATTCTGGGATATACAAATGCACAGCAAAGAACCCACTTGGTAGTGATT
ATGCAGCAACGTATATTCAAGTAATCTGACATGAAATAATAAAGTCAACAACATCTGGGCAGAATTTA
TTTTTTGGAAGAAGTTTAATCAAGGCAGCCATAGGCATGTAAATGATTTGAATAACAATTTACAGTAT
TAAATTTACAATGAACATGCAAAATAAAAGGACTTGTAAATAAATGCATTATGAACTGATGATACTGA
TTTATTTAATGGATCTCAAAACAAACTTTTAACTTAAGGCACTTTTATTTTGCCAACAAATAACAATA
AACAAACATTGAAACGGTTCACTATAAAATAACAAATGGCTAATGTACCTGAATTTTTCAGTAAAAAA
ATGAACTTCTAATACCAGTTGCCTAGTGTCCACCTCCTATCAATGTTACAAGCATGGCACTCAGAACA
GAGACAATGGAAAATATTAAATCTGCAATCTTTATGATGTAAATTTACCATCCTGATGTATAAATATT
TTGTGGTTTATAAATTTTTTTGCTAAAACCTACAGAAAAAAA

| | | | |
|---|---|---|---|
| NOV29aa, CG55776-03 | SEQ ID NO:718 | 2591 aa | MW at 287114.6 kD |
| Protein Sequence | | | |

MKVKGRGITCLLVSFAVICLVATPGGKACPRRCACYMPTEVHCTFRYLTSIPDSIPPNTERINLGYNS
LVRLMETDFSGLTKLELLMLHSNGIHTIPDKTFSDLQALQVRLMVLKMSYNKVRKLQKDTFYGLRSLT
RLHMDHNNIEFINPEVFYGLNFLRLVHLEGNQLTKLHPDTFVSLSYLQIFKISFIKFLYLSDNFLTSL
PQEMVSYMPDLDSLYLHGNPWTCDCHLKWLSDWIQEKPGIYIVLPDVIKCKKDRSPSSAQQCPLCDAP
ARTSKGKPLAMVSAAFQCAKPTIDSSLKSKSLTILEDSSSAFISPQGFMAPFGSLTLNMTDQSGNEAN
PQLYYKYKQVAPKPEDIFTNIEADLRADPSWLMQDQISLQLNRTATTFSTLQIQYSSDAQITLPRAEM
RPVKHKWTMISRDNNTKLEHTVLVGGTVGLNCPGQGDPTPHVDWLLADGSKVRAPYVSEDGRILIDKS
GKLELQMADSFDTGVYHCISSNYDDADILTYRITVVEPLVEAYQENGIHHTVFIGETLDLPCHSTGIP
DASISWVIPGNNVLYQSSRDKKVLNNGTLRILQVTPKDQGYYRCVAANPSGVDFLIFQVSVKMKGQRP
RENRRHFPPSARRIDPQHWAALLEKAKKNAMPDKRENTTVSPPPVVTQLPNIPGEEDDSSGMLALHEE
FMVPATKALNLPARTVTADSRTISDSPMTNINYGTEFSPVVNSQILPPEEPTDFKLSTAIKTTAMSKN
INPTMSSQIQGTTNQHSSTVFPLLLGATEFQDSDQMGRGREHFQSRPPITVRTMIKDVNVKMLSSTTN
KLLLESVNTTNSHQTSVREVSEPRHNHFYSHTTQILSTSTFPSDPHTAAHSQFPTPRNSTVNIPLFRR
FGRQRKIGGRGRIISPYRTPVLRRIRYSIFRSTTRGSSEKSTTAFSATKLNVTCLSCLPRERLTTATA
ALSFPSAAPITFPKADIARVPSEESTTLVQNPLLLLENKPSVEKTTPTIKYFRTEISQVTPTGAVMTY
APTSIPMEKTHKNASYPRVSSTNEAKRDSVITSSLSGAITKPPMTIIAITRFSRRKIPWQQNFVNNNH
NPKGRLRNQHKVSLQKSTAVMLPKTSPALPQRQSLPSHHTTTKTHNPGSLPTKKELPFPPLNPMLPSI
ISKDSSTKSISTQTAIPATTPTFPASVITYETQTERSIAAQTIQREQEPQKKNRTDPNISPDQSSGFT
TPTAMTPPVLTTAETSVKPSVSAFTHSPPENTTGISSTISFHSRTLNLTDVIEELAQASTQTLKSTIA
SETTLSSKSHQSTTTRKAIIRHSTIPPFLSSSATLMPVPISPPFTQRAVTDNVATPISGLMTNTVVKL
HESSRHNAKPQQLVAEVATSPKVHPNAKFTIGTTHFIYSNLLHSTPMPALTTVKSQNSKLTPSPWAEN
QFWHKPYSEIAEKGKKPEVSMLATTGLSEATTLVSDWDGQKNTKKSDFDKKPVQEATTSKLLPFDSLS
RYIFEKPRIVGGKAASFTIPANSDAFLPCEAVGNPLPTIHWTRVPSGMSGLDLSKRKQNSRVQVLPNG
TLSIQRVEIQDRGQYLCSASNLFGTDHLHVTLSVVSYPPRILEPRTKEITVHSGSTVELKCRAEGRPS
PTVTWILANQTVVSESSQGSRQAVVTVDGTLVLHNLSIYDRGFYKCVASNPGGQDSLLVKIQVIAAPP
VILEQRRQVIVGTWGESLKLPCTAKGTPQPSVYWVLSDGTEVKPLQFTNSKLFLFSNGTLYIPNLASS
DRGTYECIATSSTGSERRVVMLTMEERVTSPRIEAASQKRTEVNFGDKLLLNCSATGEPKPQIMWRLP
SKAVVDQQHRVGSWIHVYPNGSLFIGSVTEKDSGVYLCVARNKMGDDLILMHVSLELKPAKIDHKQYF
RKQVLHGKDFQVDCKASGSPVPEISWSLPDGTMINNAMQADDSGHRTRRYTLFNNGTLYFNKVGVAEE
GDYTCYAQNTLGKDEMKVHLTVITAAPRIRQSNKTNKRIKAGDTAVLDCEVIHANGSLTINKVKLLDS
GEYVCVARNPSGDDTKMYKLDVVSKPPLINGLYTNRTVIKATAVRHSKKHFDCRAEGTPSPEVMWIMP
DNIFLTAPYYGSRITVHKNGTLIRNVRLSDSADFICVARNEGGESVLLVVQLEVLEMLRRPTFRNPFN
EKIVAQLGKSTALNCSVDGNPPPEIIWILPNGTRFSNGPQSYQYLIASNGSFIISKTTREDAGKYRCA
ARNKVGYIEKLVILEIGQKPVILTYAPGTKTKGISGESLSLHCVSDGPKPNTKWTMPSGYVVDRPQIN
GKYILHDNGTLVIKEATAYDRGNYICKAQNSVGHTLITVPVMIVAYPPRRITNRPPRSIVTRTGAFQL
HCVALGVPKPEITWEMPDHSLLSTASKERTHGSEQLHLQGTLVIQNPQTSDSGIYKCTAKNPLGSDYA
ATYIQVI

| | | |
|---|---|---|
| NOV29ab, CG55776-04 | SEQ ID NO:719 | 771 bP |
| DNA Sequence | ORF Start: at 7 | ORF Stop: at 766 |

AAGCTTGCCTGTCCTCGCCGCTGTGCCTGTTATATGCCTACGGAGGTACACTGCACATTTCGGTACCT
GACTTCCATCCAGACAGCATCCCGCCCAATGTGGAACGCATCAATTTAGGATACAACAAGCTTGGTTA
GATTGATGGAAACAGATTTTTCTGGCCTGACCAAACTGGAGTTACTCATGCTTCACAGCAATGGCATT
CACACAATCCCTGACAAGACCTTCTCAGATTTGCAGGCCTTGCAGGTCTTAAAAATGAGCTATAACAA
AGTCCGAAAACTTCAGAAAGATACTTTTTATGGCCTCAGGAGCTTGACACGATTGCACATGGACCACA
ACAATATTGAGTTTATAAACCCAGAGGTTTTTGATGGGCTCAACTTTCTCCGCCTGGTGCACTTGGAA
GGAAATCAGCTCACTAAGCTCCACCCAGATACATTTGTCTCTTTGAGCTACCTCCAGATATTTAAAAT
CTCTTTCATTAAGTTCCTATACTTGTCTGATAACTTCCTGACCTCCCTCCCTCAAGAGATGGTCTCCT
ATATGCCTGACCTAGACAGCCTTTACCTGCATGGAAACCCATGGACCTGTGATTGCCATTTAAAGTGG
TTGTCTGACTGGATACAGGAGAAGCCAGATGTAATAAAATGCAAAAAAAGATAGAGTCCCTCTAGTGC
TCAGCACTGTCCACTTTGCATGAACCCTAGGACTTCTAAAGGCAAGCCGTTAGCTATGGTCTCAGCTG
CAGCTTTCCAGTGTGCCCTCGAG

| | | | |
|---|---|---|---|
| NOV29ab, CG55776-04 | SEQ ID NO:720 | 253 aa | MW at 29129.5 kD |
| Protein Sequence | | | |

ACPRRCACYMPTEVHCTFRYLTSIPDSIPPNVERINLGYNSLVRLMETDFSGLTKLELLMLHSNGIHT
IPDKTFSDLQALQVLKMSYNKVRKLQKDTFYGLRSLTRLHMDHNNIEFINPEVFDGLNFLRLVHLEGN
QLTKLHPDTFVSLSYLQIFKISFIKFLYLSDNFLTSLPQEMVSYMPDLDSLYLHGNPWTCDCHLKWLS
DWIQEKPDVIKCKKDRSPSSAQQCPLCMNPRTSKGKPLAMVSAAAFQCA

| | | |
|---|---|---|
| NOV29ac, CG55776-05 | SEQ ID NO:721 | 2011 bP |
| DNA Sequence | ORF Start: at 32 | ORF Stop: at 1988 |

AGGCTCCGCGGCCGCCCCCTTCACCCTCGAGGGGGACAAATTACTACTGAACTGCTCAGCCACTGGGG
AGCCCAAACCCCAAATAATGTGGAGGTTACCATCCAAGGCTGTGGTCGACCAGCAGCATAGAGTGGGC
AGCTGGATCCACGTCTACCCTAATGGATCCCTGTTTATTGGATCAGTAACAGAAAAAGACAGTGGTGT
CTACTTGTGTGTGGCAAGAAACAAAATGGGGGATGATCTGATACTGATGCATGTTAGCCTAAGACTGA
AACCTGCCAAATTGACCACAAGCAGTATTTTAGAPAAGCAAGTGCTCCATGGGAAGATTTCCAAAGTA
GATTGCAAGCTTCCGGCTCCCCAGTGCCAGAGATATCTTGGAAGTTTGCCTGAATGGACCATGAATCA
CATGCAAATGCAAGCCGATGACAGTGGCCACAGGACTAGGAGATATACCCTTTTCAACAATGGAACTT
TATACTTCAACAAAGTTGGGGTAAGCGGAAGGAAGGAGATTATACTTGCTATACCAGAACACCCTAAG
AAAGATGAAATGAAGGTCCACTTAACAGTTATAACAGCTGCTCCCCGGATAAGGCAGAGTAACAAAAC
CAACAAGAGAATCAAAGCTGGAGACACAGCTGTCCTTGACTGTGAGGTCACTGGGGATCCCAAACCAA
AAATATTTTGGTTGCTGCCTTCCAATGACATGATTTCCTTCTCCATTGATAGGTACACATTTCATGCC
AATGGGTCTTTGACCATCAACAAAGTGAAACTGCTCGATTCTGGAGAGTACGTATGTGTAGCCCGAAA
TCCCAGTGGGGATGAACACCAAAATGTACAACTGGATGTGGTCTCTAAACCTCCATTAATCAATGGTC
TGTATACAAACAGAAACTGTTATTAAAGCCACAGCTGTGAGACATTCCAAAAACACTTTGACTGCAGA
GCTGAAGGGACACCATCTCCTGAAGTCATGTGGATCATGCCAGACAATATTTTCCTCACAGCCCCATA
CTATGGAAGCAGAATCACAGTCCATAAAAATGGAACCTTGGAAATTAGGAATGTGAGGCTTTCAGATT
CAGCCGACTTTATCTGTGTGGCCCGAAATGAAGGTGGAGAGAGCGTGTTGGTAGTACAGTTAGAAGTA
CTGGAAATGCTGAGAAGACCGACATTTAGAATCCATTTAATGAAAAAAATAGTTGCCCAGCTGGGAAA
GTCCACAGCATTGAATTGCTCTGTTGATGGTAACCCACCACCTGAATAAATCTGGATTTTACCAAATG
GCACACGATTTTCCAATGGACCACAAAGTTATCAGTATCTGATAGCAGCAAATGGTTCTTTTATCATT
TCTAAAACAACTCGGGAGGATGCAGGAAAATATCGCTGTGCAGCTAGGAATAAAGTTGGCTATATTGA
GAAATTAGTCATATTAGAAAATTGGCCAGAAGCCAGTTATTCTTACCTATGCACCAGGGACAGTAAAG
GCATCAGTGGAGAATCTCTATCACTGCATTGTGTGTCTGATGGAATCCCTAAGCCAAATATCAAATGG
ACTATGCCAAGTGGTTATGTAGTAGACAGGCCTCAAATTAATGGGAAATACATATTGCATGACAATGG
CACCTTAGTCATTAAAGAAGCAACAGCTTATGACAGAGGAAACTATATCTGTAAGGCTCAAAATAGTG
TTGGTCATACACTGATTACTGTTCCAGTAATGATTGTAGCCTACCCTCCCCGAATTACAAATCGTCCA
CCCAGGAGTATTGTCACCAGGACAGGGGCAGCCTTTCAGCTGGACTGTGTGGCCTTGGGAGTTCCCAA
GCCAGAAATCACAATGGGAGATGCCTGACCACTCCCTTCTCTCAACGGCAGTAAAGAGAGGACACATG
GAAGTGAGCAGCTTCACTTACAAGGTACCCTAGTCATTCAGAATCCCCAAACCTCCGATTCTGGGATA
TACAAATGCACAGCACTCGAGGGCAAGGGTGGGCGCGCC

| | | | |
|---|---|---|---|
| NOV29aC, CG55776-05 | SEQ ID NO:722 | 652 aa | MW at 72283.3 kD |
| Protein Sequence | | | |

GDKLLLNCSATGEPKPQIMWRLPSKAVVDQQHRVGSWIHVYPNGSLFIGSVTEKDSGVYLCVARNKMG
DDLILMHVSLRLKPAKDHKQYFRKQKTLHGKDFQVDCKASGSPVPEISWSLPDGTMINNAMQADDSGH
RTRRYTLFNNGTLYFNKVGVAEEGDYTCYAQNTLGKDEMVHLTVITAAPRIRQSNKTNKRIKAGDTPA
VLDCEVTGDPKPKIFWLLPSNDMISFSIDRYTFHANGSLTINKVKLLDSGEYVCVARNPSGDDTKAYK
LDVVSKPPLINGLYTNRTVIKATAVRHSKKHFDCRAEGTPSPEVMWIMPDNIFLTAPYYGSRITVHKN
GTLEIRNVRLSDSADFICVAPNEGGESVLVVQLEVLEMLRRPTFRNPFNEKIVAQLGKSTALNCSVDG
NPPPEIIWILPNGTRFSNGPQSYQYLIASNGSFIISKTTREDAGKYRCAARNKVGYIEKLVILEIGQK
PVILTYAPGTVKGISGESLSLHCVSDGIPKPNIKWTMPSGYVVDRPQINGKYILHDNGTLVIKEATAY
DRGNYICKAQNSVGHTLITVPVMIVAYPPRITNRPPRSIVTRTGAAFQLHCVALGVPKPEITWEMPDH
SLLSTASKERTHGSEQLHLQGTLVIQNPQTSDSGIYKCTA

| | | |
|---|---|---|
| NOV29ad, CG55776-06 | SEQ ID NO:723 | 1975 bP |
| DNA Sequence | ORF Start: at 11 | ORF Stop: at 1967 |

CACCAGATCTGGGGACAAATTACTACTGAACTGCTCAGCCACTGGGGAGCCCAAACCCCAAATAATGT
GGAGGTTACCATCCAAGGCTGTGGTCGACCAGCAGCATAGAGTGGGCAGCTGGATCCACGTCTACCCT
AATGGATCCCTGTTTATTGGATCAGTAACAGAAAAAGACAGTGGTGTCTACTTGTGTGTGGCAAGAAA
CAAAATGGGGGATGATCTGATACTGATGCATGTTAAAGCCTAAGACTGACCTGCCAAAATTGACCACA
AGCAGTATTTAGAAAGCAAGTGCTCCATGGGAAAGATTTCCAAGTAGAATTGCAAAGCTTCCGGCTCC
CCAGTGCCAGAGATATCTTGGAGTTTGCCTGATGGAACCATGATCAACAATGCAATGCAAGCCGATGA
CAGTGGCCACAGGACTAGGAGATATACCCTTTTCACATGGAACAATTTAATACTTCAACAAGTTGGGG
TAGCGGAGGAAGGAGATTATACTTGCTATGCCCAGAACACCCTAGGGAAAGATGAAATGAAGGTCCAC
TTAACAGTTATAACAGCTGCTCCCCGGATAAGGCAGAGTAACAAAACCAACAAGAGAATCAAAGCTGG
AGACACAGCTGTCCTTGACTGTGAGGTCACTGGGGAATCCCAAACCAAAATATTTTGGTTGCTGCCTT
CCAATGACATGATTTCCTTCTCCATTGATAGGTACAACATTTCATGCCAATGGGTCTTTGACCATCAC
AAAGTGAAACTGCTCGATTCTGGAGAGTACGTAATGTGTAGCCCGATCCCAGTGGGGATGACACCAAA
AAATGTACAAACTGGATGTGGTCTCTAACCTCCAATTAATCAATGGTCTGTAATAACACAGACTGTTA
TTAAAGCCACAGCTGTGAGACATTCCAAAAAACACTTTGACTGCAGAGCTGAAGGGACACCATCTCCT
CCATAAAATGGAACCTTGGAAATTAGGAATGTGAGGCTTTCAGATTCAGCCGACTTTAATCTGTGTGG
CCCGAAATGAAGGTGGAGAGAGCGTGTTGGTAGTACAGTTAGAAGTACTGGAAATGCTGAGAAGACCG
ACATTTAGAAATCCATTTAATGAAAAAATAGTTGCCCAGCTGGGAAAGTCCACAGCATTGAATTGCTC
TGTTGATGGTAACCCACCACCTGAUATAATCTGGATTTTACCAAATGGCACACGATTTTCCAATGGAC
CACAAAGTTATCAGTATCTGATAGCAAGCAATGGTTCTTTTATCATTTCTA1KAACACTCGGGAGGAT
GCAGGAAAATATCGCTGTGCAGCTAGGAATAAAGTTGGCTATATTGAAAGATTAGTCATATTAGAAAT
TGGCCAGAAGCCAGTTATTCTTACCTATGCACCAGGGACAGTAAAAGGCATCAGTGGAGAATCTCTAT
CACTGCATTGTGTGTCTGATGGAATCCCTAAGCCAATATCAAATGGACTATGCCAAAGTGGTTATGTA
GTAGACAGGCCTCAATTAATGGGAAATAAACATATTGCATGACATGGCACCTTAGTCATTAAAGAAGC
AACAGCTTATGACAGAGGAAACTATATCTGTAAGGCTCAAATAGTGTTGGTCATACAACTGATTACTG
TTCCAGTAATGATTGTAGCCTACCCTCCCCGAATTACAAATCGTCCACCCAGGAGTATTGTCACCAGG
ACAGGGGCAGCCTTTCAGCTCCACTGTGTGGCCTTGGGAGTTCCCAAGCCAGAAATCACATGGGAGAT
AAGGTACCCTAGTCATTCAGAATCCCCAAACCTCCGATTCTGGGATATACAAATGCACAGCAGAATTC
GGC

| | | | |
|---|---|---|---|
| NOV29ad, CG55776-06 | SEQ ID NO:724 | 652 aa | MW at 72283.3 kD |
| Protein Sequence | | | |

GDKLLLNCSATGEPKPQIMWRLPSKAVVDQQHRVGSWIHVYPNGSLFIGSVTEKDSGVYLCVARNKAG
DDLILMHVSLRLKPAKIDHKQYFRKQVLHGKDFQVDCKASGSPVPEISWSLPDGTMINNAMQADDSGH
RTRRYTLFNNGTLYFNKVGVAEEGDYTCYAQNTLGKDEMKVHLTVITAAPRIRQSNKTNKRIKAGDTA
VLDCEVTGDPKPKIFWLLPSNDMISFSIDRYTFHANGSLTINKVKLLDSGEYVCVARNPSGDDTKMYK
LDWSKPPLINGLYTNRTVIKATAVRHSKKIHFDCRAEGTPSPEVMWIMPDNIFLTAPYYGSRITVHKN
GTLEIRNVRLSDSADFICVARNEGGESVLVVQLEVLEMLRRPTFRNPFNEKIVAQLGKSTALNCSVDG
NPPPEIIWILPNGTRFSNGPQSYQYLIASNGSFIISKTTREDAGKYRCAARNKVGYIEKLVILEIGQK
PVILTYAPGTVKGISGESLSLHCVSDGIPKPNIKWTMPSGYVVDRPQINGKYILHDNGTLVIKEATAY
DRGNYICKAQNSVGHTLITVPVMIVAYPPRITNRPPRSIVTRTGAAFQLHCVALGVPKPEITWEMPDH
SLLSTASKERTHGSEQLHLQGTLVTQNPQTSDSGIYKCTA

| | | |
|---|---|---|
| NOV29ae, CG55776-07 | SEQ ID NO:725 | 2902 bP |
| DNA Sequence | ORF Start: at 11 | ORF Stop: at 2894 |

CACCCTCGAGAACTCAGATGCCTTTCTTCCCTGTGAAGCTGTTGGAAATCCCCTGCCCACCATTCATT
GGACCAGAGTCCCATCAGGACTTGATTTATCTAAGAGGAAACAGAATAGCAGGGTCCAGGTTCTCCCC
AATGGTACCCTGTCCATCCAGAGGGTGGAAATTCAGGACCGCGGACAGTACTTGTGTTCCGCATCCAA
TCTGTTTGGCACAGACCACCTTCATGTCACCTTGTCTGTGGTTTCCTATCCTCCCAGGATCCTGGAGA
GACGTACCAAGAGATCACAGTTCATTCCGGAAGCACTGTGGACTGAAGTGCAGAGCAGAAAAGGTAGG
CCAAGCCCTACAGTTACCTGGATTCTTGCAAACCAAACAGTTGTCTCAGAATCATCCCAGGGAAGTAG
GCAGGCTGTGGTGACGGTTGACGGAACATTGGTCCTCCACAATCTCAGTATTTATGACCGTGGCTTTT
ACAAATGTGTGGCCAGCAACCCAGGTGGCCAGGATTCACTGCTGGTTAAAATACAAGTCATTGCAGCA
CCACCTGTTATTCTAGAGCAAAGGAGGCAAGTCATTGTAGGCACTTGGGGTGAAAGTTTAAAACTGCC
CTGTACTGCAAAGGAACTCCTCAGCCCAGCGTTTACTGGGTCCTCTCTGATGGCACTGAAAGTGAAAC
CATTACAGTTTACCAATTCCAAGTTGTTCTTATTTTCAAATGGGACTTTGTATATAAGAAACCTAGCC
TCTTCAGACAGGGGCACTTATGAATGCATTGCTACCAGTTCCACTGGTTCGGAGCGAAGAGTAGTAAT
GCTTACAATGGAAGAGCGAGTGACCAGCCCCAGGATAGAAGCTGCATCCCAGAAAAGGACTGAAGTGA
ATTTTGGGGACAAATTACTACTGAACTGCTCAGCCACTGGGGAGCCCAAACCCCAAATAATGTGGAGG
TTACCATCCAAGGCTGTGGTCGACCAGCAGCATAGAGTGGGCAGCTGGATCCACGTCTACCCTAATGG
ATCCCTGTTTATTGGATCAGTAACAGAAGACAAAGTGGTGTCTAACTTGTGTGTGGCAAGAAACAAAA
TGGGGGATGATCTGATACTGATGCATGTTAGCCTAAGACTGAAACCTGCCAAAATTGACCACAAGCAG
TATTTTAGAAAGCAAGTGCTCCATGGGAAAGATTTCCAAGTAGATTGCAAAGCTTCCGGCTCCCCAGT
GCCAGAGATATCTTGGAGTTTGCCTGATGGAACCATGATCAACAATGCAATGCAAGCCGATGACAGTG
GCCACAGGACTAGGAGATATACCCTTTTCAACAATGGAACTTTATACTTCAACAAAGTTGGGGTAGCG
GAGGAAGGAGATTATACTTGCTATGCCCAGAACACCCTAGGGAAAGATGAAATGAAGGTCCACTTAAC
CAGCTGTCCTTGACTGTGAGGTCACTGGGGATCCCAAACCAAAAATATTTTGGTTGCTGCCTTCCAAT
GACATGATTTCCTTCTCCATTGATAGGTACACATTTCATGCCAATGGGTCTTTGACCATCAAACAAGT
GAAACTGCTCGATTCTGGAGAGTACGTATGTGTAGCCCGAAATCCCAGTGGGGATGACACCAAAATGT
GCCACAGCTGTGAGACATTCCAAAAAACACTTTGACTGCAGAGCTGAAGGGACACCATCTCCTGAAGT
CATGTGGATCATGCCAGACAATATTTTCCTCACAGCCCCATACTATGGAAGCAGAATCACAGTCCATA
AAAATGGAACCTTGGAAATTAGGAATGTGAGGCTTTCAGATTCAGCCGACTTTATCTGTGTGGCCCGA
AATGAAGGTGGAGAGAGCGTGTTGGTAGTACAGTTAGAAGTACTGGAAATGCTGAGAAGACCGACATT
TAGAAATCCATTTAATGAAAAAATAGTTGCCCAGCTGGGAAAGTCCACAGCATTGAATTGCTCTGTTG
AGTTATCAGTATCTGATAGCAAGCAATGGTTCTTTTATCATTTCTAAAACAACTCGGGAGGATGCAGG
AAAATATCGCTGTGCAGCTAGGAATAAAGTTGGCTATATTGAGAAATTAGTCATATTAGAAATTGGCC
AGAAGCCAGTTATTCTTACCTATGCACCAGGGACAGTAAAAGGCATCAGTGGAGAATCTCTATCACTG
CATTGTGTGTCTGATGGAATCCCTAAGCCAAATATCAAATGGACTATGCCAAGTGGTTATGTAGTAGA
GTAATGATTGTAGCCTACCCTCCCCGAATTACAAATCGTCCACCCAGGAGTATTGTCACCAGGACAGG
GGCAAGCCTTTCAGCTCCACTGTGTGGCCTTGGGAGTTCCCAAGCCAGAATCACGTGGGAGATGCCTG
ACCACTCCCTTCTCTCAACGGCAAGTAAAGAGAGGACACATGGAAGTGAGCAGCTTCACTTAACAGGT
ACCCTAGTCATTCAGAATCCCCAAACCTCCGATTCTGGGATATACAAATGCACAGCAAAGAACCCACT
TGGTAGTGATTATGCAGCAACGTATATTCAAGTAATCCTCGAGGGC

| | | | |
|---|---|---|---|
| NOV29ae, CG55776-07 | SEQ ID NO:726 | 961 aa | MW at 106268.6 kD |
| Protein Sequence | | | |

NSDAFLPCEAVGNPLPTIHWTRVPSGLDLSKRKQNSRVQVLPNGTLSIQRVEIQDRGQYLCSASNLFG
TDHLHVTLSVVSYPPRILERRTKEITVHSGSTVELKCRAEGRPSPTVTWILANQTVTSESSQGSRQAV
VTVDGTLVLHNLSIYDRGFYKCVASNPGGQDSLLVKIQVIAAPPVILEQRRQVIVGTWGESLKLPCTA
KGTPQPSVYWVLSDGTEVKPLQFTNSKLFLFSNGTLYIRNLASSDRGTYECIATSSTGSERRVVMLTM
EERVTSPRIEAASQKRTEVNFGDKLLLNCSATGEPKPQIMWRLPSKAVVDQQHRVGSWIHVYPNGSLF
IGSVTEKDSGVYLCVARNKMGDDLILMHVSLRLKPAKIDHKQYFRKQVLHGKDFQVDCKASGSPVPEI
SWSLPDGTMINNAMQADDSGHRTRRYTLFNNGTLYFNKVGVAEEGDYTCYAQNTLGKDEMKVHLTVIT
AAPRIRQSNKTNKRIKAGDTAVLDCEVTGDPKPKIFWLLPSNDMISFSIDRYTFHANGSLTINKVKLL
DSGEYVCVARNPSGDDTKMYKLDVVSKPPLINGLYTNRTVIKATAVRHSKKHFDCREAGTPSPEVMWI
MPDNIFLTAPYYGSRITVHKINGTLEIRNVRLSDSADFICVARNEGGESVLVQLEVLEMLRRPTFRNP
FNEKIVAQLGKSTALNCSVDGNPPPEIIWILPNGTRFSNGPQSYQYLIASNGSFIISKTTREDAGKYR
CAARNKVGYIEKLVILEIGQKPVILTYAPGTVKGISGESLSLHCVSDGIPKPNIKWTMPSGYVVDRPQ
INGKYILHDNGTLVIKEATAYDRGNYICKAQNSVGHTLITVPVMIVAYPPRITNRPPRSIVTRTGAAF
QLHCVALGVPKPEITWEMPDHSLLSTASKERTHGSEQLHLQGTLVIQNPQTSDSGIYKCTAKNPLGSD
YAATYIQVI

| | | |
|---|---|---|
| NOV29af, CG55776-08 | SEQ ID NO:727 | 2902 bP |
| DNA Sequence | ORF Start: at 11 | ORF Stop: at 2894 |

CACCAGATCTAACTCAGATGCCTTTCTTCCCTGTGAAGCTGTTGGAAATCCCCTGCCCACCATTCATT
GGACCAGAGTCCCATCAGGACTTGATTTATCTAAGAGGAAACAGAATAGCAGGGTCCAGGTTCTCCCC
AATGGTACCCTGTCCATCCAGAGGGTGGAAATTCAGGACCGCGGACAGTACTTGTGTTCCGCATCCAA
TCTGTTTGGCACAGACCACCTTCATGTCACCTTGTCTGTGGTTTCCTATCCTCCCAGGATCCTGGAGA
GACGTACCAAAGAGATCACAGTTCATTCCGGAAGCACTGTGGAACTGAAGTGCAGAGCAGAAGGTAGG
CCAAGCCCTACAGTTACCTGGATTCTTGCAAACCAAACAGTTGTCTCAGAATCATCCCAGGGAAGTAG
GCAGGCTGTGGTGACGGTTGACGGAACATTGGTCCTCCACAATCTCAGTATTTATGACCGTGGCTTTT
ACAAATGTGTGGCCAGCAACCCAGGTGGCCAGGATTCACTGCTGGTTATAATACAAGTCATTGCAGCA
CCACCTGTTATTCTAGAGCAAAGGAGGCAAGTCATTGTAGGCACTTGGGGTGAAAGTTTAAAACTGCC
CTGTACTGCAAAAGGAACTCCTCAGCCCAGCGTTTACTGGGTCCTCTCTGATGGCACTGAAGTGAAAC
CATTACAGTTTACCAATTCCAAGTTGTTCTTATTTTCAAATGGGACTTTGTATATAAGAAACCTAGCC
TCTTCAGACAGGGGCACTTATGAATGCATTGCTACCAGTTCCACTGGTTCGGAGCGAAGAGTAGTAAT
GCTTACAATGGAAGAGCGAGTGACCAGCCCCAGGATAGAAGCTGCATCCCAGAAAAGGACTGAAGTGA
ATTTTGGGGACAAATTACTACTGAACTGCTCAGCCACTGGGGAGCCCAAACCCCAAATAATGTGGAGG
TTACCATCCAAGGCTGTGGTCGACCAGCAGCATAGAGTGGGCAGCTGGATCCACGTCTACCCTAATGG
ATCCCTGTTTATTGGATCAGTAACAGAAAAAGACAGTGGTGTCTACTTGTGTGTGGCAAGAAACAAAA
TGGGGGATGATCTGATACTGATGCATGTTAGCCTAAGACTGAAACCTGCCAAAATTGACCACAAGCAG
TATTTTAGAAAGCAAGTGCTCCATGGGAAAGATTTCCAAGTAGATTGCAAAGCTTCCGGCTCCCCAGT
GCCAGAGATATCTTGGAGTTTGCCTGATGGAACCATGATCAACAATGCAATGCAAGCCGATGACAGTG
GCCACAGGACTAGGAGATATACCCTTTTCAACAATGGAACTTTATACTTCAACAAAGTTGGGGTAGCG
GAGGAAGGAGATTATACTTGCTATGCCCAGAACACCCTAGGGAAAGATGAAATGAAGGTCCACTTAAC
AGTTATAACAGCTGCTCCCCGGATAAGGCAGAGTAACAAAACCAACAAGAGAATCAAAGCTGGAGACA
CAGCTGTCCTTGACTGTGAGGTCACTGGGGATCCCAAACCAAAAATATTTTGGTTGCTGCCTTCCAAT
GACATGATTTCCTTCTCCATTGATAGGTACACATTTCATGCCAATGGGTCTTTGACCATCAACAAAGT
GAAACTGCTCGATTCTGGAGAGTACGTATGTGTAGCCCGAAATCCCAGTGGGGATGACACCAAAATGT
ACAAACTGGATGTGGTCTCTAAACCTCCATTAATCAATGGTCTGTATACAAATAGAACTGTTATTAAA
CATGTGGATCATGCCAGACAATATTTTCCTCACAGCCCCATACTATGGAAGCAGAATCACAGTCCATA
AAAATGGAACCTTGGAAATTAGGAATGTGAGGCTTTCAGATTCAGCCGACTTTATCTGTGTGGCCCGA
AATGAAGGTGGAGAGAGCGTGTTGGTAGTACAGTTAGAAGTACTGGAAATGCTGAGAAGACCGACATT
TAGAAATCCATTTAATGAAAAAATAGTTGCCCAGCTGGGAAAGTCCACAGCATTGAATTGCTCTGTTG
ATGGTAACCCACCACCTGAAATAATCTGGATTTTACCAAATGGCACACGATTTTCCAATGGACCACAA
AAAATATCGCTGTGCAGCTAGGAATAAAGTTGGCTATATTGAGAAATTAGTCATATTAGAAATTGGCC
AGAAGCCAGTTATTCTTACCTATGCACCAGGGACAGTAAAAGGCATCAGTGGAGAATCTCTATCACTG
CATTGTGTGTCTGATGGAATCCCTAAGCCAAATATCAAATGGACTATGCCAAGTGGTTATGTAGTAGA
CAGGCCTCAAATTAATGGGAAATACATATTGCATGACAATGGCACCTTAGTCATTAAAGAAGCAACAG
CTTATGACAGAGGAAACTATATCTGTAAGGCTCAAAATAGTGTTGGTCATACACTGATTACTGTTCCA
GTAATGATTGTAGCCTACCCTCCCCGAATTACAAATCGTCCACCCAGGAGTATTGTCACCAGGACAGG
GGCAGCCTTTCAGCTCCACTGTGTGGCCTTGGGAGTTCCCAAGCCAGAAATCACGTGGGAGATGCCTG
ACCACTCCCTTCTCTCAACGGCAAGTAAAGAGAGGACACATGGAAGTGAGCAGCTTCACTTACAAGGT
ACCCTAGTCATTCAGAATCCCCAAACCTCCGATTCTGGGATATACAAATGCACAGCAAAGAACCCACT
TGGTAGTGATTATGCAGCAACGTATATTCAAGTAATCCTCGAGGGC

| | | | |
|---|---|---|---|
| NOV29af, CG55776-08 | SEQ ID NO:728 | 961 aa | MW at 106268.6 kD |
| Protein Sequence | | | |

NSDAFLPCEAVGNPLPTIHWTRVPSGLDLSKRKQNSRVQVLPNGTLSIQRVEIQDRGQYLCSASNLFG
TDHLHVTLSVVSYPPRILERRTKEITVHSGSTVELKCEAEGRPSPTVTWILANQTVVSESSQGSRQAV
VTVDGTLVLHNLSIYDRGFYKCVASNPGGQDSLLVKIQVIAAPPVILEQRRQVIVGTWGESLKLPCTA
KGTPQPSVYWVLSDGTEVKPLQFTNSKLFLFSNGTLYIENLASSDRGTYECIATSSTGSERRVVMLTM
EERVTSPRIEAASQKRTEVNFGDKLLLNCSATGEPKPQIMWRLPSKAVVDQQHRVGSWIHVYPNGSLF
IGSVTEKDSGVYLCVARNKMGDDLILMHVSLRLKPAKDHKQYFRKQKTLHGKDFQVDCKASGSPVPEI
SWSLPDGTMINNAMQADDSGHRTRRYTLFNNGTLYFNKVGVAEEGDYTCYAQNTLGKDEMKVHLTVIT
AAPRIRQSNKTNKRIKAGDTAVLDCEVTGDPKPKIFWLLPSNDMISFSIDRYTFHANGSLTINKVKLL
DSGEYVCVARAPSGDDTKMYKLDVVSKPPLINGLYTNRTVIKATAVRHSKKHFDCRAEGTPSPEVMWI
MPDNIFLTAPYYGSRITVHKNGTLEIRNVRLSDSADFICVARNEGGESVLVVQLEVLEMLRRPTFANP
FNEKIVAQLGKSTALNCSVDGNPPPEIIWILPNGTRFSNGPQSYQYLIASNGSFIISKTTREDAGKYR
CAARNKVGYIEKLVILEIGQKPVILTYAPGTVKGISGESLSLHCVSDGIPKPNIKWTMPSGYVVDRPQ
INGKYILHDNGTLVIKEATAYDRGNYICKAQNSVGHTLITVPVMIVAYPPRITNRPPRSIVTRTGAAF
QLHCVALGVPKPEITWEMPDHSLLSTASKERTHGSEQLHLQGTLVIQNPQTSDSGIYKCTAKNPLGSD
YAATYIQVI

| | | |
|---|---|---|
| NOV29ag, CG55776-09 | SEQ ID NO:729 | 778 bP |
| DNA Sequence | ORF Start: at 11 | ORF Stop: at 770 |

CACCGGATCCGCCTGTCCTCGCCGCTGTGCCTGTTATATGCCTACGGAGGTACACTGCACATTTCGGT
ACCTGACTTCCATCCCAGACAGCATCCCGCCCAATGTGGAACGCATCAATTTAGGATACAACAGCTTG
GTTAGATTGATGGAAACAGATTTTTCTGGCCTGACCAAACTGGAGTTACTCATGCTTCACAGCAATGG
CATTCACACAATCCCTGACAAGACCTTCTCAGATTTGCAGGCCTTGCAGGTCTTAAAAATGAGCTATA
ACAAAGTCCGAAAACTTCAGAAAGATACTTTTTATGGCCTCAGGAGCTTGACACGATTGCACATGGAC
CACAACAATATTGAGTTTATAAACCCAGAGGTTTTTGATGGGCTCAACTTTCTCCGCCTGGTGCACTT
GGAAGGAAATCAGCTCACTAAGCTCCACCCAGATACATTTGTCTCTTTGAGCTACCTCCAGATATTTA
AAATCTCTTTCATTAAGTTCCTATACTTGTCTGATAACTTCCTGACCTCCCTCCCTCAAGAGATGGTC
TCCTATATGCCTGACCTAGACAGCCTTTACCTGCATGGAAACCCATGGACCTGTGATTGCCATTTAAA
GTGGTTGTCTGACTGGATACAGGAGAAGCCAGATGTAATAAAATGCAAAAAAGATAGAAGTCCCTCTA
GTGCTCAGCAGTGTCCACTTTGCATGAACCCTAGGACTTCTAAAGGCAAGCCGTTAGCTATGGTCTCA
GCTGCAGCTTTCCAGTGTGCCCTCGAGGGC

| | | | |
|---|---|---|---|
| NOV29ag, CG55776-09 | SEQ ID NO:730 | 253 aa | MW at 29129.5 kD |

ACPRRCACYMPTEVHCTFRYLTSIPDSIPPNVERINLGYNSLVRLMETDFSGLTKLELLMLHSNGIHT
IPDKTFSDLQALQVLKMSYNKVRKLQKDTFYGLRSLTRLHMDHNNIEFINPEVFDGLNFLRLVHLEGN
QLTKLHPDTFVSLSYLQIFKISFIKFLYLSDNFLTSLPQEMVSYMPDLDSLYLHGNPWTCDCHLKWLS
DWIQEKPDVIKCKKDRSPSSAQQCPLCMNPRTSKGKPLAMVSAAAFQCA

| | | |
|---|---|---|
| NOV29ah, SNP13376522 of | SEQ ID NO:731 | 7876 bP |
| CG55776-01, | ORF Start: ATG at 6 | ORF Stop: TGA at 7857 |
| DNA Sequence | SNP Pos: 87 | SNP Change: G to A |

TCAGGATGAAGGTAAAAGGCAGAGGAATCACCTGCTTGCTGGTCTCCTTTGCTGTGATCTGCCTGGTC
GCCACCCCTGGGGGCAAGACCTGTCCTCGCCGCTGTGCCTGTTATATGCCTACGGAGGTACACTGCAC
ATTTCGGTACCTGACTTCCATCCCAGACAGCATCCCGCCCAATGTGGAACGCATCAATTTAGGGTACA
ACAGCTTGGTTAGATTGATGGAAACAGATTTTTCTGGCCTGACCAAACTGGAGTTACTCATGCTTCAC
AGCAATGGCATTCACACAATCCCTGACAAGACCTTCTCAGATTTGCAGGCCTTGCAGGTGAGACTGAT
GGTCTTAAAAATGAGCTATAATAAAGTCCGAAAACTTCAGAAAGATACTTTTTATGGCCTCAGGAGCT
TGACACGATTGCACATGGACCACAACAATATTGAGTTTATAAACCCAGAGGTTTTTTATGGGCTCAAC
TTTCTCCGCCTGGTGCACTTGGAAGGAAATCAGCTCACTAAGCTCCACCCAGATACATTTGTCTCTTT
GAGCTACCTCCAGATATTTAAAATCTCTTTCATTAAGTTCCTATACTTGTCTGATAACTTCCTGACCT
CCCTCCCTCAAGAGATGGTCTCCTATATGCCTGACCTAGACAGCCTTTACCTGCATGGAAACCCATGG
ACCTGTGATTGCCATTTAAAGTGGTTGTCTGACTGGATACAGGAGAAGCCAGGTATCTATATTGTNTT
ACCAGATGTAATAAAATGCAAAAAAGATAGAAGTCCCTCTAGTGCTCAGCAGTGTCCACTTTGCATGA
ACCCTAGGACTTCTAAAGGCAAGCCGTTAGCTATGGTCTCAGCTGCAGCTTTCCAGTGTGCCAAGCCA
ACCATTGACTCATCCCTGAAATCAAAGAGCCTGACTATTCTGGAAGACAGTAGTTCTGCTTTCATCTC
TCCCCAAGGTTTCATGGCACCCTTTGGCTCCCTCACTTTGAATATGACAGATCAGTCTGGAAATGAAG
CTAACATGGTCTGCAGTATTCAAAAGCCCTCAAGGACATCACCCATTGCATTCACTGAAGAAAATGAC
TACATCGTGCTAAATACTTCATTTTCAACATTTTTGGTGTGCAACATAGATTACGGTCACATTCAGCC
AGTGTGGCAAATTTTGGCTTTGTACAGTGATTCTCCTCTGATACTAGAAAGGAGCCACTTGCTTAGTG
AAACACCGCAGCTCTATTACAAATATAAACAGGTGGCTCCTAAGCCTGAAGACATTTTTACCAACATA
GAGGCAGATCTCAGAGCAGATCCCTCTTGGTTAATGCAAGACCAAATTTCCTTGCAGCTGAACAGAAC
TGCCACCACATTCAGTACATTACAGATCCAGTACTCCAGTGATGCTCAAATCACTTTACCAAGAGCAG
AGATGAGGCCAGTGAAACACAAATGGACTATGATTTCAAGGGATAACAATACTAAGCTGGAACATACT
GTCTTGGTAGGTGGAACCGTTGGCCTGAACTGCCCAGGCCAAGGAGACCCCACCCCACACGTGGATTG
GCTTCTAGCTGATGGAAGTAAAGTGAGAGCCCCTTATGTCAGTGAGGATGGACGGATCCTAATAGACA
AAAGTGGAAAATTGGAACTCCAGATGGCTGATAGTTTTGACACAGGCGTATATCACTGTATAAGCAGC
AATTATGATGATGCAGATATTCTCACCTATAGGATAACTGTGGTAGAACCTTTGGTCGAAGCCTATCA
GGAAAATGGGATTCATCACACAGTTTTCATTGGTGAAACACTTGATCTTCCATGCCATTCTACTGGTA
TCCCAGATGCCTCTATTAGCTGGGTTATTCCAGGAAACAATGTGCTCTATCAGTCATCAAGAGACAAG
AAAGTTCTAAACAATGGCACATTAAGAATATTACAGGTCACCCCGAAAGACCAAGGTTATTATCGCTG
TGTGGCAGCCAACCCATCAGGGGTTGATTTTTTGATTTTCCAAGTTTCAGTCA7AGATGAAAGGACAA
GGCCCTTGGAGCATGATGGAGAAACAGAGGGATCTGGACTTGATGAGTCCAATCCTATTGCTCATCTT
AAGGAGCCACCAGGTGCACAACTCCGTACATCTGCTCTGATGGAGGCTGAGGTTGGAAAACACACCTC
AAGCACAAGTAAGAGGCACAACTATCGGGAATTAACACTCCAGCGACGTGGAGATTCAACACATCGAC
GTTTTAGGGAGAATAGGAGGCATTTCCCTCCCTCTGCTAGGAGAATTGACCCACAACATTGGGCGGCA
CTGTTGGAGAAAGCTAAAAAGAATGCTATGCCAGACAAGCGAGAAAATACCACAGTGAGCCCACCCCC
AGTGGTCACCCAACTCCCAAACATACCTGGTGAAGAAGACGATTCCTCAGGCATGCTCGCTCTACATG
AGGAATTTATGGTCCCGGCCACTAAAGCTTTGAACCTTCCAGCAAGGACAGTGACTGCTGACTCCAGA
ACAATATCTGATAGTCCTATGACAAACATAAATTATGGCACAGAATTCTCTCCTGTTGTGAATTCACA
AATACTACCACCTGAAGAACCCACAGATTTCAAACTGTCTACTGCTATTAAAACTACAGCCATGTCAA
AGAATATAAACCCAACCATGTCAAGCCAAATACAAGGCACAACCAATCAACATTCATCCACTGTCTTT
CCACTGCTACTTGGAGCAACTGAATTTCAGGACTCTGACCAGATGGGAAGAGGAAGAGAGCATTTCCA
AAGTAGACCCCCAATAACAGTAAGGACTATGATCAAAGATGTCAATGTCAAAATGCTTAGTAGCACCA
CCAACAAACTATTATTAGAGTCAGTAAATACCACAAATAGTCATCAGACATCTGTAAGAGAAGTGAGT
GAACCCAGGCACAATCACTTCTATTCTCACACTACTCAAATACTTAGCACCTCCACGTTCCCTTCAGA
TCCACACACAGCTGCTCATTCTCAGTTTCCGATCCCTAGAAATAGTACAGTTAACATCCCGCTGTTCA
GACGCTTTGGGAGGCAGAGGAAAATTGGCGGAAGGGGGCGGATTATCAGCCCATATAGAACTCCAGTT
CTGCGACGGCATAGATACAGCATTTTCAGGTCAACAACCAGAGGTTCTTCTGAAAAAAGCACTACTGC
ATTCTCAGCCACAGTGCTCAATGTGACATGTCTGTCCTGTCTTCCCAGGGAGAGGCTCACCACTGCCA
CAGCAGCATTGTCTTTTCCAAGTGCTGCTCCCATCACCTTCCCCAAAGCTGACATTGCTAGAGTCCCA
TCAGAAGAGTCTACAACTCTAGTCCAGAATCCACTATTACTACTTGAGAACAAACCCAGTGTAGAGAA
AACAACACCCACAATAAAATATTTCAGGACTGAAATTTCCCAAGTGACTCCAACTGGTGCAGTCATGA
CATATGCTCCAACATCCATACCCATGGAAAAAACTCACAAAGTAAACGCCAGTTACCCACGTGTGTCT
AGCACCAATGAAGCTAAAAGAGATTCAGTGATTACATCGTCACTTTCAGGTGCTATCACCAAGCCACC
AATGACTATTATAGCCATTACAAGGTTTTCAAAGAAGGAAATTCCCTGGCAACAGAACTTTGTAAATA
ACCATAACCCAAAGGCAGATTAAGGAATCAAACATAAAGTTAGTTTACAA3AAAGCACAGCTGTGATG
CTTCCTAAACATCTCCTGCTTTACCACAGAGAACAAAGTCTCCCCTCGCACCACACTACGACCAAAAC
ACACAATCCTGGAAGTCTTCCAACAAAGAAGGAGCTTCCCTTCCCACCCCTTAACCCTATGCTTCCTA
GTATTATAAGCAAAGACTCAAGTACAAIAAGCATCATATCAACGCAAACAGCAATACCAGCAACAACT
CCTACCTTCCCTGCATCTGTCATCACTTATGAAACCCAAACAGAGAGATCTAGAGCACAAACAATACA
AAGAGAACAGGAGCCTCAAGAAGAACAGGAACTGAACCCAAACATCTCTCCAGACCAGAGTTCTGGCT
TCACTACACCCACTGCTATGACACCTCCTGTTCTAACCACAGCCGAAACTTCAGTCAAGCCCAGTGTC
TCTGCATTCACTCATTCCCCACCAGAAAACACAACTGGGATTTCAAGCACAATCAGTTTTCATTCAAG
ACTCTTAATCTGACAGATGTGATTGAIAGAACTAGCCCAAGCAAGTACTCAGACTTTGAAGAGCACAA
TTGCTTCTGAACAACTTTGTCCAGCAAATCAACACCAGAGTACCACAACTAGGAAAGCAATCATTAGA
CACTCAACCATACCACCATTCTTGAGCAGCAGTGCTACTCTAATGCCAGTTCCCATCTCCCCTCCCTT
AAGGTTCACCCAAATGCCAAGTTCACAATTGGAACCACTCACTTCATCTACTCTAATCTGTTACATTC
TACTCCCATGCCAGCAACTAACAACAGTTAATCACAGAATTCTAAATTAACTCCATCTCCCTGGGCAG
AAAACCAATTTTGGCACAAACCATACTCAGAAATTGCTGAAAAAGGCAAAAAGCCAGAAGTAAGCATG
TTGGCTACTACAGGCCTGTCCGAGGCCACCACTCTTGTTTCAGATTGGGATGGACAGAAGAACACAAA
GAAGAGTGACTTTGATAAGAAACCAGTTCAAGAAGCAACAACTTCCAAACTCCTTCCCTTTGACTCTT
TGTCTAGGTATATATTTGAAAAGCCCAGGATAGTTGGAGGAAAAGCTGCAAGTTTTACTATTCCAGCT
CCCATCAGGTATGTCAGGACTTGATTTATCTAAGAGGAAACAGAATAGCAGGGTCCAGGTTCTCCCCA
ATGGTACCCTGTCCATCCAGAGGGTGGAAATTCAGGACCGCGGACAGTACTTGTGTTCCGCATCCAAT
CTGTTTGGCACAGACCACCTTCATGTCACCTTGTCTGTGGTTTCCTATCCTCCCAGGATCCTGGAGAG
ACGTACCAAAGAGATCACAGTTCATTCCGGAAGCACTGTGGAACTGAAGTGCAGAGCAGAAGGTAGGC
CAAGCCCTACAGTTACCTGGATTCTTGCACCAAACAAAGTTGTCTCAGAATCATCCCAGGGAAGTAGG
CAGGCTGTGGTGACGGTTGACGGAACATTGGTCCTCCACAATCTCAGTATTTATGACCGTGGCTTTTA
CAAATGTGTGGCCAGCAACCCAGGTGGCCAGGATTCACTGCTGGTTAAAATACAAGTCATTGCAGCAC
CACCTGTTATTCTAGAGCAAAGGAGGCAAGTCATTGTAGGCACTTGGGGTGAAAGTTTAAAACTGCCC
TGTACTGCAAAAGGAACTCCTCAGCCCAGCGTTTACTGGGTCCTCTCTGATGGCACTGAAGTGAAACC
ATTACAGTTTACCAATTCCAAGTTGTTCTTATTTTCAAATGGGACTTTGTATATAAGAAACCTAGCCT
CTTCAGACAGGGGCACTTATGAATGCATTGCTACCAGTTCCACTGGTTCGGAGCGAAGAGTAGTAATG
CTTACAATGGAAGAGCGAGTGACCAGCCCCAGGATAGAAGCTGCATCCCAGAAAAGGACTGAAGTGAA
TTTTGGGGACAAATTACTACTGAACTGCTCAGCCACTGGGGAGCCCAAACCCCAAATAATGTGGAGGT
TACCATCCAAGGCTGTGGTCGACCAGCAGCATAGGGTGGGCAGCTGGATCCACGTCTACCCTAATGGA
GGGGGATGATCTGATACTGATGCATGTTAGCCTAAGACTGAAACCTGCCAAAATTGACCACAAGCAGT
ATTTTAGAAAGCAAGTGCTCCATGGGAAAGATTTCCAAGTAGATTGCAAAGCTTCCGGCTCCCCAGTG
CCAGAGATATCTTGGAGTTTGCCTGATGGAACCATGATCAACAATGCAATGCAAGCCGATGACAGTGG
AGGAAGGAGATTATACTTGCTATGCCCAGAACACCCTAGGGAAAGATGAAATGAAGGTCCACTTAACA
GTTATAACAGCTGCTCCCCGGATAAGGCAGAGTAACAAAACCAACAAGAGAATCAAAGCTGGAGACAC
AGCTGTCCTTGACTGTGAGGTCACTGGGGATCCCAAACCAAAAATATTTTGGTTGCTGCCTTCCAATG
ACATGATTTCCTTCTCCATTGATAGGTACACATTTCATGCCAATGGGTCTTTGACCATCAACAAAGTG
AAACTGCTCGATTCTGGAGAGTACGTATGTGTAGCCCGAAATCCCAGTGGGGATGACACCAAAATGTA
CAAACTGGATGTGGTCTCTAAACCTCCATTAATCAATGGTCTGTATACAAACAGAACTGTTATTAAAG
CCACAGCTGTGAGACATTCCAAAAAACACTTTGACTGCAGAGCTGAAGGGACACCATCTCCTGAAGTC
ATGTGGATCATGCCAGACAATATTTTCCTCACAGCCCCATACTATGGAAGCAGAATCACAGTCCATAA
AAATGGAACCTTGGAAATTAGGAATGTGAGGCTTTCAGATTCAGCCGACTTTATCTGTGTGGCCCGAA
ATGAAGGTGGAGAGAGCGTGTTGGTAGTACAGTTAGAAGTACTGGAAATGCTGAGAAGACCGACATTT
AGAAATCCATTTAATGAAAAAATAGTTGCCCAGCTGGGAAAGTCCACAGCATTGAATTGCTCTGTTGA
TGGTAACCCACCACCTGAAATAATCTGGATTTTACCAAATGGCACACGATTTTCCAATGGACCACAAA
GTTATCAGTATCTGATAGCAAGCAATGGTTCTTTTATCATTTCTAAAACAACTCGGGAGGATGCAGGA
AAATATCGCTGTGCAGCTAGGAATAAAGTTGGCTATATTGAGAAATTAGTCATATTAGAAATTGGCCA
GAAGCCAGTTATTCTTACCTATGCACCAGGGACAGTAAAAGGCATCAGTGGAGAATCTCTATCACTGC
ATTGTGTGTCTGATGGAATCCCTAAGCCAAATATCAAATGGACTATGCCAAGTGGTTATGTAGTAGAC
AGGCCTCAAATTAATGGGAAATACATATTGCATGACAATGGCACCTTAGTCATTAAAGAAGCAACAGC
TTATGACAGAGGAAACTATATCTGTAAGGCTCAAAATAGTGTTGGTCATACACTGATTACTGTTCCAG
TAATGATTGTAGCCTACCCTCCCCGAATTACAAATCGTCCACCCAGGAGTATTGTCACCAGGACAGGG
GCAGCCTTTCAGCTCCACTGTGTGGCCTTGGGAGTTCCCAAGCCAGAAATCACATGGGAGATGCCTGA
CCACTCCCTTCTCTCAACGGCAAGTAAAGAGAGGACACATGGAAGTGAGCAGCTTCACTTACAAGGTA
CCCTAGTCATTCAGAATCCCCAAACCTCCGATTCTGGGATATACAAATGCACAGCAAAGAACCCACTT
GGTAGTGATTATGCAGCAACGTATATTCAAGTAATCTGACATGAAATAATAAAGTC

| | | | |
|---|---|---|---|
| NOV29ah, SNP13376522 of | SEQ ID NO:732 | 2617 aa | MW at 290230.2 kD |
| CG55776-01, Protein Sequence | SNP Pos: 28 | | SNP Change: |
| | | | Ala to Thr |

MKVKGRGITCLLVSFAVICLVATPGGKTCPRRCACYMPTEVHCTFRYLTSIPDSIPPNVERINLGYNS
LVRLMETDFSGLTKLELLMLHSNGIHTIPDKTFSDLQALQVRLMVLKNSYNKVRKLQKDTFYGLRSLT
RLHMDHNNIEFINPEVFYGLNFLRLVHLEGNQLTKLHPDTFVSLSYLQIFKISFIKFLYLSDNFLTSL
PQEMVSYMPDLDSLYLHGNPWTCDCHLKWLSDWIQEKPGIYIVLPDVIKCKKDRSPSSAQQCPLCMNP
RTSKGKPLAMVSAAAFQCAKPTIDSSLKSKSLTILEDSSSAFISPQGFMAPFGSLTLNMTDQSGNEAN
MVCSIQKPSRTSPIAFTEENDYIVLNTSFSTFLVCNIDYGHIQPVWQILALYSDSPLILERSHLLSET
PQLYYKYKQVAPKPEDIFTNIEADLRADPSWLMQDQISLQLNRTATTFSTLQIQYSSDAQITLPRAEM
RPVKHKWTMISRDNNTKLEHTVLVGGTVGLNCPGQGDPTPHVDWLLADGSKVEAPYVSEDGRILIDKS
GKLELQMADSFDTGVYHCISSNYDDADILTYRITVVEPLVEAYQENGIHHTVFIGETLDLPCHSTGIP
DASISWVIPGNAVLYQSSRDKKVLNNGTLRILQVTPKDQGYYRCVAANPSGVDFLIFQVSVKMKGQRP
LEHDGETEGSGLDESNPIAHLKEPPGAQLRTSALMEAEVGKHTSSTSKRHNYRELTLQRRGDSTHRRF
RENRRHFPPSARRIDPQHWAALLEKAKKNAMPDKRENTTVSPPPVVTQLPNIPGEEDDSSGMLALHEE
FMVPATKALNLPARTVTADSRTISDSPMTNINYGTEFSPVVNSQILPPEEPTDFKLSTAIKTTAMSKN
INPTMSSQIQGTTNQHSSTVFPLLLGATEFQDSDQMGRGREHFQSRPPITVRTMIKDVNVKMLSSTTN
KLLLESVNTTNSHQTSVREVSEPRHNHFYSHTTQILSTSTFPSDPHTAAHSQFPIPRNSTVNIPLFRR
FGRQRKIGGRGRIISPYRTPVLRRHRYSIFRSTTRGSSEKSTTAFSATVLNVTCLSCLPRERLTTATA
ALSFPSAAPITFPKADIARVPSEESTTLVQNPLLLLENKPSVEKTTPTIKYFRTEISQVTPTGAVMTY
APTSIPMEKTHKVNASYPRVSSTNEAKRDSVITSSLSGAITKPPMTIIAITRFSRRKIPWQQNFVNNH
NPKGRLRNQHKVSLQKSTAVMLPKTSPALPQRQSLPSHHTTTKTHNPGSLPTKKELPFPPLNPMLPSI
ISKDSSTKSIISTQTAIPATTPTFPASVITYETQTERSRAQTIQREQEPQKKNRTDPNISPDQSSGFT
TPTAMTPPVLTTAETSVKPSVSAFTHSPPENTTGISSTISFHSRTLNLTDVIEELAQASTQTLKSTIA
SETTLSSKSHQSTTTRKAIIRHSTIPPFLSSSATLMPVPISPPFTQRAVTDNVATPISGLMTNTVVKL
HESSRHNAKPQQLVAEVATSPKVHPNAKFTIGTTHFIYSNLLHSTPMPALTTVKSQNSKLTPSPWAEN
QFWHKPYSEIAEKGKKPEVSMLATTGLSEATTLVSDWDGQKNTKKSDFDKKPVQEATTSKLLPFDSLS
RYIFEKPRIVGGKAASFTIPANSDAFLPCEAVGNPLPTIHWTRVPSGMSGLDLSKRKQNSRVQVLPNG
TLSIQRVEIQDRGQYLCSASNLFGTDHLHVTLSVVSYPPRILERRTKEITVHSGSTVELKCRAEGRPS
PTVTWILANQTVVSESSQGSRQAVVTVDGTLVLHNLSIYDRGFYKCVASNPGGQDSLLVKTQVIAAPP
DRGTYECIATSSTGSERRVVMLTMEERVTSPRIEAASQKRTEVNFGDKLLLNCSATGEPKPQIMWRLP
SKAVVDQQHRVGSWIHVYPNGSLFIGSVTEKDSGVYLCVARNKMGDDLILMHVSLRLKPAKIDHKQYF
RKQVLHGKDFQVDCKASGSPVPEISWSLPDGTMINNAMQADDSGHRTRRYTLFNNGTLYFNKVGVAEE
GDYTCYAQNTLGKDEMKVHLTVITAAPRIRQSNKTNKRIKAGDTAVLDCEVTGDPKPKIFWLLPSNDM
ISFSIDRYTFHANGSLTINKVKLLDSGEYVCVARNPSGDDTKMYKLDVVSKPPLINGLYTNRTVIKAT
AVRHSKKHFDCRAEGTPSPEVMWIMPDNIFLTAPYYGSRITVHKNGTLEIRNVRLSDSADFICVARNE
GGESVLVVQLEVLEMLRRPTFRNPFNEKIVAQLGKSTALNCSVDGNPPPEIIWILPNGTRFSNGPQSY
QYLIASNGSFIISKTTREDAGKYRCAARNKVGYIEKLVILEIGQKPVILTYAPGTVKGISGESLSLHC
VSDGIPKPNIKWTMPSGYVVDRPQINGKYILHDNGTLVIKEATAYDRGNYICKAQNSVGHTLITVPVM
IVAYPPRITNRPPRSIVTRTGAAFQLHCVALGVPKPEITWEMPDHSLLSTASKERTHGSEQLHLQGTL
VIQNPQTSDSGIYKCTAKNPLGSDYAATYIQVI

| | | |
|---|---|---|
| NOV29ai, SNP13377624 | SEQ ID NO:733 | 7876 bP |
| of CG55776-01, | ORF Start: ATG at 6 | ORF Stop: TGA at 7857 |
| DNA Sequence | SNP Pos: 113 | SNP Change: T to C |

TCAGGATGAAGGTAAAAGGCAGAGGAATCACCTGCTTGCTGGTCTCCTTTGCTGTGATCTGCCTGGTC
GCCACCCCTGGGGGCAAGGCCTGTCCTCGCCGCTGTGCCTGTTACATGCCTACGGAGGTACACTGCAC
ATTTCGGTACCTGACTTCCATCCCAGACAGCATCCCGCCCAATGTGGAACGCATCAATTTAGGGTACA
ACAGCTTGGTTAGATTGATGGAAACAGATTTTTCTGGCCTGACCAAACTGGAGTTACTCATGCTTCAC
AGCAATGGCATTCACACAATCCCTGACAAGACCTTCTCAGATTTGCAGGCCTTGCAGGTGAGACTGAT
GGTCTTAIAAATGAGCTATAATAAAGTCCGAAAACTTCAGAAAGATACTTTTTATGGCCTCAGGAGCT
TGACACGATTGCACATGGACCACAACAATATTGAGTTTATAAACCCAGAGGTTTTTTATGGGCTCAAC
TTTCTCCGCCTGGTGCACTTGGAAGGAAATCAGCTCACTAAGCTCCACCCAGATACATTTGTCTCTTT
GAGCTACCTCCAGATATTTAAAATCTCTTTCATTAAGTTCCTATACTTGTCTGATAACTTCCTGACCT
CCCTCCCTCAAGAGATGGTCTCCTATATGCCTGACCTAGACAGCCTTTACCTGCATGGAAACCCATGG
ACCTGTGATTGCCATTTAAAGTGGTTGTCTGACTGGATACAGGAGAAGCCAGGTATCTATATTGTNTT
ACCAGATGTAATAAAATGCAAAAAAGATAGAAGTCCCTCTAGTGCTCAGCAGTGTCCACTTTGCATGA
ACCCTAGGACTTCTAAAGGCAAGCCGTTAGCTATGGTCTCAGCTGCAGCTTTCCAGTGTGCCAAGCCA
ACCATTGACTCATCCCTGAAATCAAAGAGCCTGACTATTCTGGAAGACAGTAGTTCTGCTTTCATCTC
TCCCCAAGGTTTCATGGCACCCTTTGGCTCCCTCACTTTGAATATGACAGATCAGTCTGGAAATGAAG
CTAACATGGTCTGCAGTATTCAAAAGCCCTCAAGGACATCACCCATTGCATTCACTGAAGAAAATGAC
TACATCGTGCTAAATACTTCATTTTCAACATTTTTGGTGTGCAACATAGATTACGGTCACATTCAGCC
AGTGTGGCAAATTTTGGCTTTGTACAGTGATTCTCCTCTGATACTAGAAAGGAGCCACTTGCTTAGTG
AAACACCGCAGCTCTATTACAAATATAAACAGGTGGCTCCTAAGCCTGAAGACATTTTTACCAACATA
GAGGCAGATCTCAGAGCAGATCCCTCTTGGTTAATGCAAGACCAAATTTCCTTGCAGCTGAACAGAAC
TGCCACCACATTCAGTACATTACAGATCCAGTACTCCAGTGATGCTCAAATCACTTTACCAAGAGCAG
AGATGAGGCCAGTGAAACACAAATGGACTATGATTTCAAGGGATAACAATACTAAGCTGGAACATACT
GTCTTGGTAGGTGGAACCGTTGGCCTGAACTGCCCAGGCCAAGGAGACCCCACCCCACACGTGGATTG
AAAGTGGAAAATTGGAACTCCAGATGGCTGATAGTTTTGACACAGGCGTATATCACTGTATAAGCAGC
AATTATGATGATGCAGATATTCTCACCTATAGGATAACTGTGGTAGAACCTTTGGTCGAAGCCTATCA
GGAAAATGGGATTCATCACACAGTTTTCATTGGTGAAACACTTGATCTTCCATGCCATTCTACTGGTA
TCCCAGATGCCTCTATTAGCTGGGTTATTCCAGGAAACAATGTGCTCTATCAGTCATCAAGAGACAAG
TGTGGCAGCCAACCCATCAGGGGTTGATTTTTTGATTTTCCAAGTTTCAGTCAAGATGAAAGGACAAA
GGCCCTTGGAGCATGATGGAGAAACAGAGGGATCTGGACTTGATGAGTCCAATCCTATTGCTCATCTT
AAGGAGCCACCAGGTGCACAACTCCGTACATCTGCTCTGATGGAGGCTGAGGTTGGAAAACACACCTC
GTTTTAGGGAGAATAGGAGGCATTTCCCTCCCTCTGCTAGGAGAATTGACCCACAACATTGGGCGGCA
CTGTTGGAGAAAGCTAAAAAGAATGCTATGCCAGACAAGCGAGAAAATACCACAGTGAGCCCACCCCC
AGTGGTCACCCAACTCCCAAACATACCTGGTGAAGAAGACGATTCCTCAGGCATGCTCGCTCTACATG
AGGAATTTATGGTCCCGGCCACTAAAGCTTTGAACCTTCCAGCAAGGACAGTGACTGCTGACTCCAGA
ACAATATCTGATAGTCCTATGACAAACATAAATTATGGCACAGAATTCTCTCCTGTTGTGAATTCACA
AATACTACCACCTGAAGAACCCACAGATTTCAAACTGTCTACTGCTATTAAAACTACAGCCATGTCAA
AGAATATAAACCCAACCATGTCAAGCCAAATACAAGGCACAACCAATCAACATTCATCCACTGTCTTT
CCACTGCTACTTGGAGCAACTGAATTTCAGGACTCTGACCAGATGGGAAGAGGAAGAGAGCATTTCCA
AAGTAGACCCCCAATAACAGTAAGGACTATGATCAAAGATGTCAATGTCAAAATGCTTAGTAGCACCA
GAACCCAGGCACAATCACTTCTATTCTCACACTACTCAAATACTTAGCACCTCCACGTTCCCTTCAGA
TCCACACACAGCTGCTCATTCTCAGTTTCCGATCCCTAGAAATAGTACAGTTAACATCCCGCTGTTCA
GACGCTTTGGGAGGCAGAGGAAAATTGGCGGAAGGGGGCGGATTATCAGCCCATATAGAACTCCAGTT
CTGCGACGGCATAGATACAGCATTTTCAGGTCAACAACCAGAGGTTCTTCTGAAAAAAGCACTACTGC
ATTCTCAGCCACAGTGCTCAATGTGACATGTCTGTCCTGTCTTCCCAGGGAGAGGCTCACCACTGCCA
CAGCAGCATTGTCTTTTCCAAGTGCTGCTCCCATCACCTTCCCCAAAGCTGACATTGCTAGAGTCCCA
TCAGAAGAGTCTACAACTCTAGTCCAGAATCCACTATTACTACTTGAGAACAAACCCAGTGTAGAGAA
AACAACACCCACAATAAAATATTTCAGGACTGAAATTTCCCAAGTGACTCCAACTGGTGCAGTCATGA
CATATGCTCCAACATCCATACCCATGGAIAAAACTCACAAAGTAAACGCCAGTTACCCACGTGTGTCT
AGCACCAATGAAGCTAAAAGAGATTCAGTGATTACATCGTCACTTTCAGGTGCTATCACCAAGCCACC
AATGACTATTATAGCCATTACAAGGTTTTCAAGAAGGAAAATTCCCTGGCAACAGAACTTTGTAAATA
ACCATAACCCAAAAGGCAGATTAAGGAATCAACATAAAGTTAGTTTACAAAAAAGCACAGCTGTGATG
CTTCCTAAAACATCTCCTGCTTTACCACAGAGACAAAGTCTCCCCTCGCACCACACTACGACCAAAAC
ACACAATCCTGGAAGTCTTCCAACAAAGAAGGAGCTTCCCTTCCCACCCCTTAACCCTATGCTTCCTA
GTATTATAAGCAAAGACTCAAGTACAAAAAGCATCATATCAACGCAAACAGCAATACCAGCAACAACT
CCTACCTTCCCTGCATCTGTCATCACTTATGAAACCCAAACAGAGAGATCTAGAGCACAAACAATACA
AAGAGAACAGGAGCCTCAAAAGAAGAACAGGACTGACCCAAACATCTCTCCAGACCAGAGTTCTGGCT
TCACTACACCCACTGCTATGACACCTCCTGTTCTAACCACAGCCGAAACTTCAGTCAAGCCCAGTGTC
TCTGCATTCACTCATTCCCCACCAGAAAACACAACTGGGATTTCAAGCACAATCAGTTTTCATTCAAG
AACTCTTAATCTGACAGATGTGATTGAAGAACTAGCCCAAGCAAGTACTCAGACTTTGAAGAGCACAA
TTGCTTCTGAAACAACTTTGTCCAGCAAATCACACCAGAGTACCACAACTAGGAAAGCAATCATTAGA
CACTCAACCATACCACCATTCTTGAGCAGCAGTGCTACTCTAATGCCAGTTCCCATCTCCCCTCCCTT
TACTCAGAGAGCAGTTACTGACAACGTGGCGACTCCCATTTCCGGGCTTATGACAAATACAGTGGTCA
AGCTGCACGAATCCTCAAGGCACAATGCTAAACCACAGCAATTAGTAGCAGAGGTTGCAACATCCCCC
AAGGTTCACCCAAATGCCAAGTTCACAATTGGAACCACTCACTTCATCTACTCTAATCTGTTACATTC
TACTCCCATGCCAGCACTAACAACAGTTAAATCACAGAATTCTAAATTAACTCCATCTCCCTGGGCAG
AAAACCAATTTTGGCACAAACCATACTCAGAAATTGCTGAAAAAGGCAAAAAGCCAGAAGTAAGCATG
TTGGCTACTACAGGCCTGTCCGAGGCCACCACTCTTGTTTCAGATTGGGATGGACAGAAGAACACAAA
GAAGAGTGACTTTGATAAGAAACCAGTTCAAGAAGCAACAACTTCCAAACTCCTTCCCTTTGACTCTT
TGTCTAGGTATATATTTGAAAAGCCCAGGATAGTTGGAGGAAAAGCTGCAAGTTTTACTATTCCAGCT
AACTCAGATGCCTTTCTTCCCTGTGAAGCTGTTGGAAATCCCCTGCCCACCATTCATTGGACCAGAGT
CCCATCAGGTATGTCAGGACTTGATTTATCTAAGAGGAAACAGAATAGCAGGGTCCAGGTTCTCCCCA
ATGGTACCCTGTCCATCCAGAGGGTGGAAATTCAGGACCGCGGACAGTACTTGTGTTCCGCATCCAAT
CTGTTTGGCACAGACCACCTTCATGTCACCTTGTCTGTGGTTTCCTATCCTCCCAGGATCCTGGAGAG
ACGTACCAAAGAGATCACAGTTCATTCCGGAAGCACTGTGGAACTGAAGTGCAGAGCAGAAGGTAGGC
CAAGCCCTACAGTTACCTGGATTCTTGCAAACCAAACAGTTGTCTCAGAATCATCCCAGGGAAATAGG
CAGGCTGTGGTGACGGTTGACGGAACATTGGTCCTCCACAATCTCAGTATTTATGACCGTGGCTTTTA
CAAATGTGTGGCCAGCAACCCAGGTGGCCAGGATTCACTGCTGGTTAAAATACAAGTCATTGCAGCAC
CACCTGTTATTCTAGAGCAAAGGAGGCAAGTCATTGTAGGCACTTGGGGTGAAAGTTTAAAACTGCCC
TGTACTGCAAAAGGAACTCCTCAGCCCAGCGTTTACTGGGTCCTCTCTGATGGCACTGAAGTGAAACC
ATTACAGTTTACCAATTCCAAGTTGTTCTTATTTTCAAATGGGACTTTGTATATAAGAAACCTAGCCT
CTTCAGACAGGGGCACTTATGAATGCATTGCTACCAGTTCCACTGGTTCGGAGCGAAGAGTAGTAATG
CTTACAATGGAAGAGCGAGTGACCAGCCCCAGGATAGAAGCTGCATCCCAGAAAAGGACTGAAGTGAA
TTTTGGGGACAAATTACTACTGAACTGCTCAGCCACTGGGGAGCCCAAACCCCAAATAATGTGGAGGT
TACCATCCAAGGCTGTGGTCGACCAGCAGCATAGGGTGGGCAGCTGGATCCACGTCTACCCTAATGGA
TCCCTGTTTATTGGATCAGTAACAGAAAAAGACAGTGGTGTCTACTTGTGTGTGGCAAGAAACAAAAT
GGGGGATGATCTGATACTGATGCATGTTAGCCTAAGACTGAAACCTGCCAAAATTGACCACAAGCAGT
ATTTTAGAAAGCAAGTGCTCCATGGGAAAGATTTCCAAGTAGATTGCAAAGCTTCCGGCTCCCCAGTG
CCAGAGATATCTTGGAGTTTGCCTGATGGAACCATGATCAACAATGCAATGCAAGCCGATGACAGTGG
CCACAGGACTAGGAGATATACCCTTTTCAACAATGGAACTTTATACTTCAACAAAGTTGGGGTAGCGG
AGGAAGGAGATTATACTTGCTATGCCCAGAACACCCTAGGGAAAGATGAAATGAAGGTCCACTTAACA
GTTATAACAGCTGCTCCCCGGATAAGGCAGAGTAACAAAACCAACAAGAGAATCAAAGCTGGAGACAC
ACATGATTTCCTTCTCCATTGATAGGTACACATTTCATGCCAATGGGTCTTTGACCATCAACAAAGTG
AAACTGCTCGATTCTGGAGAGTACGTATGTGTAGCCCGAAATCCCAGTGGGGATGACACCAAAATGTA
CAAACTGGATGTGGTCTCTAAACCTCCATTAATCAATGGTCTGTATACAAACAGAACTGTTATTAAAG
CCACAGCTGTGAGACATTCCAAAAAACACTTTGACTGCAGAGCTGAAGGGACACCATCTCCTGAAGTC
ATGTGGATCATGCCAGACAATATTTTCCTCACAGCCCCATACTATGGAAGCAGAATCACAGTCCATAA
AAATGGAACCTTGGAAATTAGGAATGTGAGGCTTTCAGATTCAGCCGACTTTATCTGTGTGGCCCGAA
ATGAAGGTGGAGAGAGCGTGTTGGTAGTACAGTTAGAAGTACTGGAAATGCTGAGAAGACCGACATTT
TGGTAACCCACCACCTGAAATAATCTGGATTTTACCAAATGGCACACGATTTTCCAATGGACCACAAA
GTTATCAGTATCTGATAGCAAGCAATGGTTCTTTTATCATTTCTAAAACAACTCGGGAGGATGCAGGA
AAATATCGCTGTGCAGCTAGGAATAAAGTTGGCTATATTGAGAAATTAGTCATATTAGAAATTGGCCA
GAAGCCAGTTATTCTTACCTATGCACCAGGGACAGTAAAAGGCATCAGTGGAGAATCTCTATCACTGC
ATTGTGTGTCTGATGGAATCCCTAAGCCAAATATCAAATGGACTATGCCAAGTGGTTATGTAGTAGAC
AGGCCTCAAATTAATGGGAAATACATATTGCATGACAATGGCACCTTAGTCATTAAAGAAGCAACAGC
TTATGACAGAGGAAACTATATCTGTAAGGCTCAAAATAGTGTTGGTCATACACTGATTACTGTTCCAG
TAATGATTGTAGCCTACCCTCCCCGAATTACAAATCGTCCACCCAGGAGTATTGTCACCAGGACAGGG
GCAGCCTTTCAGCTCCACTGTGTGGCCTTGGGAGTTCCCAAGCCAGAAATCACATGGGAGATGCCTGA
CCACTCCCTTCTCTCAACGGCAAGTAAAGAGAGGACACATGGAAGTGAGCAGCTTCACTTACAAGGTA
CCCTAGTCATTCAGAATCCCCAAACCTCCGATTCTGGGATATACAAATGCACAGCAAAGAACCCACTT
GGTAGTGATTATGCAGCAACGTATATTCAAGTAATCTGACATGAAATAATAAAGTC

| | | | |
|---|---|---|---|
| NOV29ai, SNP13377624 of | SEQ ID NO:734 | 2617 aa | MW at 2902002 kD |
| CG55776-01, Protein Sequence | SNP Pos:36 | | SNP Change: Tyr to Tyr |

MKVKGRGITCLLVSFAVICLVATPGGKACPRRCACYMPTEVHCTFRYLTSIPDSIPPNVERINLGYNS
LVRLMETDFSGLTKLELLMLHSNGIHTIPDKTFSDLQALQVRLMVLKMSYNKVRKLQKDTFYGLRSLT
RLHMDHNNIEFINPEVFYGLNFLRLVHLEGNQLTKLHPDTFVSLSYLQIFKISFIKFLYLSDNFLTSL
PQEMVSYMPDLDSLYLHGNPWTCDCHLKWLSDWIQEKPGIYIVLPDVIKCKKDRSPSSAQQCPLCDAP
RTSKGKPLAMVSAAAFQCAKPTIDSSLKSKSLTILEDSSSAFISPQGFMAPFGSLTLNMTDQSGNEAN
MVCSIQKPSRTSPIAFTEENDYIVLNTSFSTFLVCNIDYGHIQPVWQILALYSDSPLILERSHLLSET
PQLYYKYKQVAPKPEDIFTNIEADLRADPSWLMQDQISLQLNRTATTFSTLQIQYSSDAQITLPRAEM
RPVKHKWTMISRDNNTKLEHTVLVGGTVGLNCPGQGDPTPHVDWLLADGSKVRAPYVSEDGRILIDKS
GKLELQMADSFDTGVYHCISSNYDDADILTYRITVVEPLVEAYQENGIHHTVFIGETLDLPCHSTGIP
DASISWVIPGNNVLYQSSRDKKVLNNGTLRILQVTPKDQGYYRCVAANPSGVDFLIFQVSVKMKGQRP
LEHDGETEGSGLDESNPIAHLKEPPGAQLRTSALMEAEVGKHTSSTSKRHNYRELTLQRRGDSTHRRF
RENRRHFPPSARRIDPQHWAALLEKAKKNAMPDKRENTTVSPPPVVTQLPNIPGEEDDSSGMLALHEE
FMVPATKALNLPARTVTADSRTISDSPMTNINYGTEFSPVVNSQILPPEEPTDFKLSTAIKTTAMSKN
INPTMSSQIQGTTNQHSSTVFPLLLGATEFQDSDQMGRGREHFQSRPPITVRTMIKDVNVKMLSSTTN
KLLLESVNTTNSHQTSVREVSEPRHNHFYSHTTQILSTSTFPSDPHTAAHSQFPIPRNSTVNIPLFRR
FGRQRKIGGRGRIISPYRTPVLRRHRYSIFRSTTRGSSEKSTTAFSATVLNVTCLSCLPRERLTTATA
ALSFPSAAPITFPKADIARVPSEESTTLVQNPLLLLENKPSVEKTTPTIKYFRTEISQVTPTGAVMTY
APTSIPMEKTHKVNASYPRVSSTNEAKRDSVITSSLSGAITKPPMTIIAITRFSRRKIPWQQNFVNNH
NPKGRLPNQHKVSLQKSTAVMLPKTSPALPQRQSLPSHHTTTKTHNPGSLPTKKELPFPPLNPMLPSI
ISKDSSTKSIISTQTAIPATTPTFPASVITYETQTERSRAQTIQREQEPQKKNRTDPNISPDQSSGFT
TPTAMTPPVLTTAETSVKPSVSAFTHSPPENTTGISSTISFHSRTLNLTDVIEELAQASTQTLKSTIA
SETTLSSKSHQSTTTRKAIIRHSTIPPFLSSSATLMPVPISPPFTQRAVTDNVATPISGLMTNTVVKL
HESSRHNAKPQQLVAEVATSPKVHPNAKFTIGTTHFIYSNLLHSTPMPALTTVKSQNSKLTPSPWAEN
QFWHKPYSEIAEKGKKPEVSMLATTGLSEATTLVSDWDGQKNTKKSDFDKKPVQEATTSKLLPFDSLS
RYIFEKPRIVGGKAASFTIPANSDAFLPCEAVGNPLPTIHWTRVPSGMSGLDLSKRKQNSRVQVLPNG
TLSIQRVEIQDRGQYLCSASNLFGTDHLHVTLSVVSYPPRILERRTKEITVHSGSTVELKCRAEGRPS
PTVTWILANQTVVSESSQGSRQAVVTVDGTLVLHNLSIYDRGFYKCVASNPGGQDSLLVKIQVIAAPP
VILEQRRQVIVGTWGESLKLPCTAKGTPQPSVYWVLSDGTEVKPLQFTNSKLFLFSNGTLYIPNLASS
DRGTYECIATSSTGSERRVVMLTMEERVTSPRIEAASQKRTEVNFGDKLLLNCSATGEPKPQIMWRLP
SKAVVDQQHRVGSWIHVYPNGSLFIGSVTEKDSGVYLCVARNKMGDDLILMHVSLRLKPAKIDHKQYF
RKQVLHGKDFQVDCKASGSPVPEISWSLPDGTMINNAMQADDSGHRTRRYTLFNNGTLYFNKVGVAEE
GDYTCYAQNTLGKDEMKVHLTVITAAPRIRQSNKTNKRIKAGDTAVLDCEVTGDPKPKIFWLLPSNDM
ISFSIDRYTFHANGSLTINKVKLLDSGEYVCVARNPSGDDTKMYKLDVVSKPPLINGLYThRTVIKAT
AVRHSKKHFDCRAEGTPSPEVMWIMPDNIFLTAPYYGSRITVHKNGTLEIRNVRLSDSADFICVARNE
GGESVLVVQLEVLEMLRRPTFRNPFNEKIVAQLGKSTALNCSVDGNPPPEIIWILPNGTRFSNGPQSY
QYLIASNGSFIISKTTREDAGKYRCAARNKVGYIEKLVILEIGQKPVILTYAPGTVKGISGESLSLHC
VSDGIPKPNIKWTMPSGYVVDRPQINGKYILHDNGTLVIKEATAYDRGNYICKAQNSVGHTLITVPVM
IVAYPPRITNRPPRSIVTRTGAAFQLHCVALGVPKPEITWEMPDHSLLSTASKERTHGSEQLHLQGTL
VIQNPQTSDSGIYKCTAKNPLGSDYAATYIQVI

| | | |
|---|---|---|
| NOV29a, SNP13377625 | SEQ ID NO:735 | 7876 bP |
| of CG55776-01, | ORF Start: ATG at 6 | ORF Stop: TGA at 7857 |
| DNA Sequence | SNP PoS: 161 | SNP Change: A to G |

TCAGGATGAAGGTAAAAGGCAGAGGAATCACCTGCTTGCTGGTCTCCTTTGCTGTGATCTGCCTGGTC
GCCACCCCTGGGGGCAAGGCCTGTCCTCGCCGCTGTGCCTGTTATATGCCTACGGAGGTACACTGCAC
ATTTCGGTACCTGACTTCCATCCCGGACAGCATCCCGCCCAATGTGGAACGCATCAATTTAGGGTACA
ACAGCTTGGTTAGATTGATGGAAACAGATTTTTCTGGCCTGACCAAACTGGAGTTACTCATGCTTCAC
TGACACGATTGCACATGGACCACAACAATATTGAGTTTATAAACCCAGAGGTTTTTTATGGGCTCAAC
TTTCTCCGCCTGGTGCACTTGGAAGGAAATCAGCTCACTAAGCTCCACCCAGATACATTTGTCTCTTT
GAGCTACCTCCAGATATTTAAAATCTCTTTCATTAAGTTCCTATACTTGTCTGATAACTTCCTGACCT
ACCTGTGATTGCCATTTAAAGTGGTTGTCTGACTGGATACAGGAGAAGCCAGGTATCTATATTGTNTT
ACCAGATGTAATAAAATGCAAAAAAGATAGAAGTCCCTCTAGTGCTCAGCAGTGTCCACTTTGCATGA
ACCCTAGGACTTCTAAAGGCAAGCCGTTAGCTATGGTCTCAGCTGCAGCTTTCCAGTGTGCCAAGCCA
ACCATTGACTCATCCCTGAAATCAAAGAGCCTGACTATTCTGGAAGACAGTAGTTCTGCTTTCATCTC
TCCCCAAGGTTTCATGGCACCCTTTGGCTCCCTCACTTTGAATATGACAGATCAGTCTGGAAATGAAG
CTAACATGGTCTGCAGTATTCAAAAGCCCTCAAGGACATCACCCATTGCATTCACTGAAGAAAATGAC
TACATCGTGCTAAATACTTCATTTTCACAATTTTTGGTGTGCAACATAGATTACGGTCACATTCAGCC
AGTGTGGCAAATTTTGGCTTTGTACAGTGATTCTCCTCTGATACTAGAAAGGAGCCACTTGCTTAGTG
AAACACCGCAGCTCTATTACAAATATAAACAGGTGGCTCCTAAGCCTGAAGACATTTTTACCAACATA
GAGGCAGATCTCAGAGCAGATCCCTCTTGGTTAATGCAAGACCAAATTTCCTTGCAGCTGAACAGAAC
TGCCACCACATTCAGTACATTACAGATCCAGTACTCCAGTGATGCTCAAATCACTTTACCAAGAGCAG
AGATGAGGCCAGTGAAACACAAATGGACTATGATTTCAAGGGATAACAATACTAAGCTGGAACATACT
GTCTTGGTAGGTGGAACCGTTGGCCTGAACTGCCCAGGCCAAGGAGACCCCACCCCACACGTGGATTG
GCTTCTAGCTGATGGAAGTAAAGTGAGAGCCCCTTATGTCAGTGAGGATGGACGGATCCTAATAGACA
AAAGTGGAAAATTGGAACTCCAGATGGCTGATAGTTTTGACACAGGCGTATATCACTGTATAAGCAGC
GGAAAATGGGATTCATCACACAGTTTTCATTGGTGAAACACTTGATCTTCCATGCCATTCTACTGGTA
TCCCAGATGCCTCTATTAGCTGGGTTATTCCAGGAAACAATGTGCTCTATCAGTCATCAAGAGACAAG
AAAGTTCTAAACAATGGCACATTAAGAATATTACAGGTCACCCCGAAAGACCAAGGTTATTATCGCTG
TGTGGCAGCCAACCCATCAGGGGTTGATTTTTTGATTTTCCAAGTTTCAGTCAAGATGAAAGGACAAA
GGCCCTTGGAGCATGATGGAGAAACAGAGGGATCTGGACTTGATGAGTCCAATCCTATTGCTCATCTT
AAGGAGCCACCAGGTGCACAACTCCGTACATCTGCTCTGATGGAGGCTGAGGTTGGAAAACACACCTC
AAGCACAAGTAAGAGGCACAACTATCGGGAATTAACACTCCAGCGACGTGGAGATTCAACACATCGAC
GTTTTAGGGAGAATAGGAGGCATTTCCCTCCCTCTGCTAGGAGAATTGACCCACAACATTGGGCGGCA
CTGTTGGAGAAAGCTAAAAAGAATGCTATGCCAGACAAGCGAGAAAATACCACAGTGAGCCCACCCCC
AGTGGTCACCCAACTCCCAAACATACCTGGTGAAGAAGACGATTCCTCAGGCATGCTCGCTCTACATG
AGGAATTTATGGTCCCGGCCACTAAAGCTTTGAACCTTCCAGCAAGGACAGTGACTGCTGACTCCAGA
ACAATATCTGATAGTCCTATGACAAACATAAATTATGGCACAGAATTCTCTCCTGTTGTGAATTCACA
AATACTACCACCTGAAGAACCCACAGATTTCAAACTGTCTACTGCTATTAAAACTACAGCCATGTCAA
AGAATATAAACCCAACCATGTCAAGCCAMATACAAGGCACAACCAATCAACATTCATCCACTGTCTTT
CCACTGCTACTTGGAGCAACTGAATTTCAGGACTCTGACCAGATGGGAAGAGGAAGAGAGCATTTCCA
AAGTAGACCCCCAATAACAGTAAGGACTATGATCAAAGATGTCAATGTCAAAATGCTTAGTAGCACCA
CCAACAAACTATTATTAGAGTCAGTAAATACCACAAATAGTCATCAGACATCTGTAAGAGAAGTGAGT
GAACCCAGGCACAATCACTTCTATTCTCACACTACTCAAATACTTAGCACCTCCACGTTCCCTTCAGA
TCCACACACAGCTGCTCATTCTCAGTTTCCGATCCCTAGAAATAGTACAGTTAACATCCCGCTGTTCA
GACGCTTTGGGAGGCAGAGGAAAATTGGCGGAAGGGGGCGGATTATCAGCCCATATAGAACTCCAGTT
CTGCGACGGCATAGATACAGCATTTTCAGGTCAACAACCAGAGGTTCTTCTGAAAAAAGCACTACTGC
ATTCTCAGCCACAGTGCTCAATGTGACATGTCTGTCCTGTCTTCCCAGGGAGAGGCTCACCACTGCCA
CAGCAGCATTGTCTTTTCCAAGTGCTGCTCCCATCACCTTCCCCAAAGCTGACATTGCTAGAGTCCCA
TCAGAAGAGTCTACAACTCTAGTCCAGAATCCACTATTACTACTTGAGAACAAACCCAGTGTAGAGAA
AACAACACCCACAATAAAATATTTCAGGACTGAAATTTCCCAAGTGACTCCAACTGGTGCAGTCATGA
CATATGCTCCAACATCCATACCCATGGAAAAAACTCACAAAGTAAACGCCAGTTACCCACGTGTGTCT
AGCACCAATGAAGCTAAAAGAGATTCAGTGATTACATCGTCACTTTCAGGTGCTATCACCAAGCCACC
AATGACTATTATAGCCATTACAAGGTTTTCAAGAAGGAAAATTCCCTGGCAACAGAACTTTGTAAATA
ACCATAACCCAAAAGGCAGATTAAGGAATCAACATAAAGTTAGTTTACAAAAAAGCACAGCTGTGATG
CTTCCTAAAACATCTCCTGCTTTACCACAGAGACAAAGTCTCCCCTCGCACCACACTACGACCAAAAC
ACACAATCCTGGAAGTCTTCCAACAAAGAAGGAGCTTCCCTTCCCACCCCTTAACCCTATGCTTCCTA
GTATTATAAGCAAAGACTCAAGTACAAAAAGCATCATATCAACGCAAACAGCAATACCAGCAACAACT
AAGAGAACAGGAGCCTCAAAAGAAGAACAGGACTGACCCAAACATCTCTCCAGACCAGAGTTCTGGCT
TCACTACACCCACTGCTATGACACCTCCTGTTCTAACCACAGCCGAAACTTCAGTCAAGCCCAGTGTC
AACTCTTAATCTGACAGATGTGATTGAAGAACTAGCCCAAGCAAGTACTCAGACTTTGAAGAGCACAA
TTGCTTCTGAAACAACTTTGTCCAGCAAATCACACCAGAGTACCACAACTAGGAAAGCAATCATTAGA
TACTCAGAGAGCAGTTACTGACAACGTGGCGACTCCCATTTCCGGGCTTATGACAAATACAGTGGTCA
AGCTGCACGAATCCTCAAGGCACAATGCTAAACCACAGCAATTAGTAGCAGAGGTTGCAACATCCCCC
AAGGTTCACCCAAATGCCAAGTTCACAATTGGAACCACTCACTTCATCTACTCTAATCTGTTACATTC
TACTCCCATGCCAGCACTAACAACAGTTAAATCACAGAATTCTAAATTAACTCCATCTCCCTGGGCAG
AAAACCAATTTTGGCACAAACCATACTCAGAAATTGCTGAAAAAGGCAAAAAGCCAGAAGTAAGCATG
TTGGCTACTACAGGCCTGTCCGAGGCCACCACTCTTGTTTCAGATTGGGATGGACAGAAGAACACAAA
GAAGAGTGACTTTGATAAGAAACCAGTTCAAGAAGCAACAACTTCCAAACTCCTTCCCTTTGACTCTT
TGTCTAGGTATATATTTGAAAAGCCCAGGATAGTTGGAGGAAAAGCTGCAAGTTTTACTATTCCAGCT
CCCATCAGGTATGTCAGGACTTGATTTATCTAAGAGGAAACAGAATAGCAGGGTCCAGGTTCTCCCCA
ATGGTACCCTGTCCATCCAGAGGGTGGAAATTCAGGACCGCGGACAGTACTTGTGTTCCGCATCCAAT
CTGTTTGGCACAGACCACCTTCATGTCACCTTGTCTGTGGTTTCCTATCCTCCCAGGATCCTGGAGAG
ACGTACCAAAGAGATCACAGTTCATTCCGGAAGCACTGTGGAACTGAAGTGCAGAGCAGAAGGTAGGC
CAAGCCCTACAGTTACCTGGATTCTTGCAAACCAAACAGTTGTCTCAGAATCATCCCAGGGAAGTAGG
CAGGCTGTGGTGACGGTTGACGGAACATTGGTCCTCCACAATCTCAGTATTTATGACCGTGGCTTTTA
CAAATGTGTGGCCAGCAACCCAGGTGGCCAGGATTCACTGCTGGTTAAAATACAAGTCATTGCAGCAC
CACCTGTTATTCTAGAGCAAAGGAGGCAAGTCATTGTAGGCACTTGGGGTGAAAGTTTAAAACTGCCC
TGTACTGCAAAAGGAACTCCTCAGCCCAGCGTTTACTGGGTCCTCTCTGATGGCACTGAAGTGAAACC
ATTACAGTTTACCAATTCCAAGTTGTTCTTATTTTCAAATGGGACTTTGTATATAAGAAACCTAGCCT
CTTCAGACAGGGGCACTTATGAATGCATTGCTACCAGTTCCACTGGTTCGGAGCGAAGAGTAGTAATG
CTTACAATGGAAGAGCGAGTGACCAGCCCCAGGATAGAAGCTGCATCCCAGAkAAGGACTGAAGTGAA
TTTTGGGGACAAATTACTACTGAACTGCTCAGCCACTGGGGAGCCCAAACCCCAAATAATGTGGAGGT
TACCATCCAAGGCTGTGGTCGACCAGCAGCATAGGGTGGGCAGCTGGATCCACGTCTACCCTAATGGA
TCCCTGTTTATTGGATCAGTAACAGAAAAAGACAGTGGTGTCTACTTGTGTGTGGCAAGAAACAAAAT
GGGGGATGATCTGATACTGATGCATGTTAGCCTAAGACTGAAACCTGCCAAAATTGACCACAAGCAGT
ATTTTAGAAAGCAAGTGCTCCATGGGAAAGATTTCCAAGTAGATTGCAAAGCTTCCGGCTCCCCAGTG
CCAGAGATATCTTGGAGTTTGCCTGATGGAACCATGATCAACAATGCAATGCAAGCCGATGACAGTGG
CCACAGGACTAGGAGATATACCCTTTTCAACAATGGAACTTTATACTTCAACAAAGTTGGGGTAGCGG
AGGAAGGAGATTATACTTGCTATGCCCAGAACACCCTAGGGAAAGATGAAATGAAGGTCCACTTAACA
GTTATAACAGCTGCTCCCCGGATAAGGCAGAGTAACAAAACCAACAAGAGAATCAAAGCTGGAGACAC
AGCTGTCCTTGACTGTGAGGTCACTGGGGATCCCAAACCAALAATATTTTGGTTGCTGCCTTCCAATG
ACATGATTTCCTTCTCCATTGATAGGTACACATTTCATGCCAATGGGTCTTTGACCATCAACAAAGTG
AAACTGCTCGATTCTGGAGAGTACGTATGTGTAGCCCGAAATCCCAGTGGGGATGACACCAAAATGTA
CAAACTGGATGTGGTCTCTAAACCTCCATTAATCAATGGTCTGTATACAAACAGAACTGTTATTAAAG
CCACAGCTGTGAGACATTCCAAAAAACACTTTGACTGCAGAGCTGAAGGGACACCATCTCCTGAAGTC
ATGTGGAATCATGCCAGACATATTTTCCTCACAGCCCCATACTATGGAAAGCAGATCACAGTCCATAA
AAATGGAACCTTGGAAATTAGGAATGTGAGGCTTTCAGATTCAGCCGACTTTATCTGTGTGGCCCGAA
ATGAAGGTGGAGAGAGCGTGTTGGTAGTACAGTTAGAAGTACTGGAAATGCTGAGAAGACCGACATTT
AGAAATCCATTTAATGAAAAAATAGTTGCCCAGCTGGGAAAGTCCACAGCATTGAATTGCTCTGTTGA
TGGTAACCCACCACCTGAAATAATCTGGATTTTACCAAATGGCACACGATTTTCCAATGGACCACAAA
GTTATCAGTATCTGATAGCAAGCAATGGTTCTTTTATCATTTCTAAAACAACTCGGGAGGATGCAGGA
AAATATCGCTGTGCAGCTAGGAATAAAGTTGGCTATATTGAGAAATTAGTCATATTAGAAATTGGCCA
GAAGCCAGTTATTCTTACCTATGCACCAGGGACAGTAAAAGGCATCAGTGGAGAATCTCTATCACTGC
ATTGTGTGTCTGATGGAATCCCTAAGCCAAATATCAAATGGACTATGCCAAGTGGTTATGTAGTAGAC
AGGCCTCATTAIATGGGAAATACATATTGCATGACAATGGCACCTTAGTCATTAAAGAAAGCAACAGC
TTATGACAGAGGAAACTATATCTGTAAGGCTCAAAATAGTGTTGGTCATACACTGATTACTGTTCCAG
TAATGATTGTAGCCTACCCTCCCCGAATTACAAATCGTCCACCCAGGAGTATTGTCACCAGGACAGGG
GCAGCCTTTCAGCTCCACTGTGTGGCCTTGGGAGTTCCCAAGCCAGAAATCACATGGGAGATGCCTGA
CCACTCCCTTCTCTCAACGGCAAGTAAAGAGAGGACACATGGAAGTGAGCAGCTTCACTTACAAGGTA
CCCTAGTCATTCAGAATCCCCAAACCTCCGATTCTGGGATATACAAATGCACAGCAAAGAACCCACTT
GGTAGTGATTATGCAGCAACGTATATTCAGTAATCTGACATGAATAATAGTC

| | | | |
|---|---|---|---|
| NOV29a, SNP13377625 of | SEQ ID NO:736 | 2617 aa | MW at 290200.2 kD |
| CG55776-01, Protein Sequence | ASNP PoS: 52 | | SNP Change: |
| | | | Pro to Pro |

MKVKGRGITCLLVSFAVICLVATPGGKACPRRCACYMPTEVHCTFRYLTSIPDSIPPNVERINLGYNS
LVRLMETDFSGLTKLELLMLHSNGIHTIPDKTFSDLQALQVRLMVLKMSYNKVRKLQKDTFYGLRSLT
RLHMDHNNIEFINPEVFYGLNFLRLVHLEGNQLTKLHPDTFVSLSYLQIFKISFIKFLYLSDNFLTSL
PQEMVSYMPDLDSLYLHGNPWTCDCHLKWLSDWIQEKPGIYIVLPDVIKCKKDRSPSSAQQCPLCMNP
RTSKGKPLAMVSAAAFQCAKPTIDSSLKSKSLTILEDSSSAFISPQGFMAPFGSLTLNMTDQSGNEAN
MVCSIQKPSRTSPIAFTEENDYIVLNTSFSTFLVCNIDYGHIQPVWQILALYSDSPLILERSHLLSET
PQLYYKYKQVAPKPEDIFTNIEADLRADPSWLMQDQISLQLNRTATTFSTLQIQYSSDAQITLPRAEM
RPVKHKWTMISRDNNTKLEHTVLVGGTVGLNCPGQGDPTPHVDWLLADGSKVRAPYVSEDGRILIDKS
GKLELQMADSFDTGVYHCISSNYDDADILTYRITVVEPLVEAYQENGIHHTVFIGETLDLPCHSTGIP
DASISWVIPGNNVLYQSSRDKKVLNNGTLRILQVTPKDQGYYRCVAANPSGVDFLIFQVSVKMKGQRP
LEHDGETEGSGLDESNPIAHLKEPPGAQLRTSALMEAEVGKHTSSTSKRHNYRELTLQRRGDSTHRRF
RENRRHFPPSARRIDPQHWAALLEKAKKNAMPDKRENTTVVSPPPWTQLPNIPGEEDDSSGMLALHEE
FMVPATKALNLPARTVTADSRTISDSPMTNINYGTEFSPVVNSQILPPEEPTDFKLSTAIKTTAMSKN
INPTMSSQIQGTTNQHSSTVFPLLLGATEFQDSDQMGRGREHFQSRPPITVRTMIKDVNVKMLSSTTN
KLLLESVNTTNSHQTSVREVSEPRHNHFYSHTTQILSTSTFPSDPHTAAHSQFPIPRNSTVNIPLFRR
FGRQRKIGGRGRIISPYRTPVLRRHRYSIFRSTTRGSSEKSTTAFSATVLNVTCLSCLPRERLTTATA
ALSFPSAAPITFPKADIARVPSEESTTLVQNPLLLLENKPSVEKTTPTIKYFRTEISQVTPTGAVMTY
APTSIPMEKTHKVNASYPRVSSTNEAKRDSVITSSLSGAITKPPMTIIAITRFSRRKIPWQQNFVNNH
NPKGRLRNQHKVSLQKSTAVMLPKTSPALPQRQSLPSHHTTTKTHNPGSLPTKKELPFPPLNPMLPSI
ISKDSSTKSIISTQTAIPATTPTFPASVITYETQTERSRAQTIQREQEPQKKNRTDPNISPDQSSGFT
TPTAMTPPVLTTAETSVKPSVSAFTHSPPENTTGISSTISFHSRTLNLTDVIEELAQASTQTLKSTIA
SETTLSSKSHQSTTTRKAIIRHSTIPPFLSSSATLMPVPISPPFTQRAVTDNVATPISGLMTNTVVKL
HESSRHNAKPQQLVAEVATSPKVHPNAKFTIGTTHFIYSNLLHSTPMPALTTVKSQNSKLTPSPWAEN
QFWHKPYSEIAEKGKKPEVSMLATTGLSEATTLVSDWDGQKNTKKSDFDKKPVQEATTSKLLPFDSLS
RYIFEKPRIVGGKAASFTIPANSDAFLPCEAVGNPLPTIHWTRVPSGMSGLDLSKRKQNSRVQVLPNG
TLSIQRVEIQDRGQYLCSASNLFGTDHLHVTLSVVSYPPRILERRTKEITVHSGSTVELKCRAEGRPS
PTVTWILANQTVVSESSQGSRQAVVTVDGTLVLHNLSIYDRGFYKCVASNPGGQDSLLVKIQVIAAPP
VILEQRRQVIVGTWGESLKLPCTAKGTPQPSVYWVLSDGTEVKPLQFTNSKLFLFSNGTLYIRNLASS
DRGTYECIATSSTGSERRVVMLTMEERVTSPRIEAASQKRTEVNFGDKLLLNCSATGEPKPQIMWRLP
SKAVVDQQHRVGSWIHVYPNGSLFIGSVTEKDSGVYLCVARNKMGDDLILMHVSLRLKPAKIDHKQYF
RKQVLHGKDFQVDCKASGSPVPEISWSLPDGTMINNAMQADDSGHRTRRYTLFNNGTLYFNKVGVAEE
GDYTCYAQNTLGKDEMKVHLTVITAAPRIRQSNKTNKRIKAGDTAVLDCEVTGDPKPKIFWLLPSNDM
ISFSIDRYTFHANGSLTINKVKLLDSGEYVCVARNPSGDDTKMYKLDVVSKPPLINGLYTNRTVIKAT
AVRHSKKHFDCPAEGTPSPEVMWIMPDNIFLTAPYYGSRITVHKNGTLEIRNVRLSDSADFICVARNE
GGESVLVVQLEVLEMLRRPTFRNPFNEKIVAQLGKSTALNCSVDGNPPPEIIWILPNGTRFSNGPQSY
QYLIASNGSFIISKTTREDAGKYRCAARNKVGYIEKLVILEIGQKPVILTYAPGTVKGISGESLSLHC
VSDGIPKPNTKWTMPSGYVVDRPQINGKYILHDNGTLVIKEATAYDRGNYICKAQNSVGHTLITVPVM
IVAYPPRITNRPPRSIVTRTGAAFQLHCVALGVPKPEITWEMPDHSLLSTASKERTHGSEQLHLQGTL
VIQNPQTSDSGIYKCTAKNPLGSDYAATYIQVI

| | | |
|---|---|---|
| NOV29ak, SNP13376523 of | SEQ ID NO:737 | 7876 bP |
| CG55776-01, | ORF Start: ATG at 6 | ORF Stop: TGA at 7857 |
| DNA Sequence | SNP Pos: 187 | SNP Change: G to A |

TCAGGATGAAGGTAAAAGGCAGAGGAATCACCTGCTTGCTGGTCTCCTTTGCTGTGATCTGCCTGGTC
GCCACCCCTGGGGGCAAGGCCTGTCCTCGCCGCTGTGCCTGTTATATGCCTACGGAGGTACACTGCAC
ATTTCGGTACCTGACTTCCATCCCAGACAGCATCCCGCCCAATGTGGAACACATCAATTTAGGGTACA
ACAGCTTGGTTAGATTGATGGAAACAGATTTTTCTGGCCTGACCAAACTGGAGTTACTCATGCTTCAC
AGCAATGGCATTCACACAATCCCTGACAAGACCTTCTCAGATTTGCAGGCCTTGCAGGTGAGACTGAT
GGTCTTAAAAATGAGCTATAATAAAGTCCGAAAACTTCAGAAAGATACTTTTTATGGCCTCAGGAGCT
TGACACGATTGCACATGGACCACAACAATATTGAGTTTATAAACCCAGAGGTTTTTTATGGGCTCAAC
TTTCTCCGCCTGGTGCACTTGGAAGGAAATCAGCTCACTAAGCTCCACCCAGATACATTTGTCTCTTT
GAGCTACCTCCAGATATTTAAAATCTCTTTCATTAAGTTCCTATACTTGTCTGATAACTTCCTGACCT
CCCTCCCTCAAGAGATGGTCTCCTATATGCCTGACCTAGACAGCCTTTACCTGCATGGAAACCCATGG
ACCTGTGATTGCCATTTAAAGTGGTTGTCTGACTGGATACAGGAGAAGCCAGGTATCTATATTGTNTT
ACCAGATGTAATAAAATGCAAAAAAGATAGAAGTCCCTCTAGTGCTCAGCAGTGTCCACTTTGCATGA
ACCCTAGGACTTCTAAAGGCAAGCCGTTAGCTATGGTCTCAGCTGCAGCTTTCCAGTGTGCCAAGCCA
ACCATTGACTCATCCCTGAAATCAAAGAGCCTGACTATTCTGGAAGACAGTAGTTCTGCTTTCATCTC
TCCCCAAGGTTTCATGGCACCCTTTGGCTCCCTCACTTTGAATATGACAGATCAGTCTGGAAATGAAG
CTAACATGGTCTGCAGTATTCAAAAGCCCTCAAGGACATCACCCATTGCATTCACTGAAGAAAATGAC
TACATCGTGCTAAATACTTCATTTTCAACATTTTTGGTGTGCAACATAGATTACGGTCACATTCAGCC
AGTGTGGCAAATTTTGGCTTTGTACAGTGATTCTCCTCTGATACTAGAAAGGAGCCACTTGCTTAGTG
AAACACCGCAGCTCTATTACAAATATAAACAGGTGGCTCCTAAGCCTGAAGACATTTTTACCAACATA
TGCCACCACATTCAGTACATTACAGATCCAGTACTCCAGTGATGCTCAAATCACTTTACCAAGAGCAG
AGATGAGGCCAGTGAAACACAAATGGACTATGATTTCAAGGGATAACAATACTAAGCTGGAACATACT
GTCTTGGTAGGTGGAACCGTTGGCCTGAACTGCCCAGGCCAAGGAGACCCCACCCCACACGTGGATTG
GCTTCTAGCTGATGGAAGTAAAGTGAGAGCCCCTTATGTCAGTGAGGATGGACGGATCCTAATAGACA
AAAGTGGAAAATTGGAACTCCAGATGGCTGATAGTTTTGACACAGGCGTATATCACTGTATAAGCAGC
AATTATGATGATGCAGATATTCTCACCTATAGGATAACTGTGGTAGAACCTTTGGTCGAAGCCTATCA
TCCCAGATGCCTCTATTAGCTGGGTTATTCCAGGAAACAATGTGCTCTATCAGTCATCAAGAGACAAG
AAAGTTCTAAACAATGGCACATTAAGAATATTACAGGTCACCCCGAAAGACCAAGGTTATTATCGCTG
TGTGGCAGCCAACCCATCAGGGGTTGATTTTTTGATTTTCCAAGTTTCAGTCAAGATGAAAGGACAAA
GGCCCTTGGAGCATGATGGAGAAACAGAGGGATCTGGACTTGATGAGTCCAATCCTATTGCTCATCTT
AAGGAGCCACCAGGTGCACAACTCCGTACATCTGCTCTGATGGAGGCTGAGGTTGGAAAACACACCTC
AAGCACAAGTAAGAGGCACAACTATCGGGAATTAACACTCCAGCGACGTGGAGATTCAACACATCGAC
GTTTTAGGGAGAATAGGAGGCATTTCCCTCCCTCTGCTAGGAGAATTGACCCACAACATTGGGCGGCA
CTGTTGGAGAAAGCTAAAAAGAATGCTATGCCAGACAAGCGAGAAAATACCACAGTGAGCCCACCCCC
AGTGGTCACCCAACTCCCAAACATACCTGGTGAAGAAGACGATTCCTCAGGCATGCTCGCTCTACATG
AGGAATTTATGGTCCCGGCCACTAAAGCTTTGAACCTTCCAGCAAGGACAGTGACTGCTGACTCCAGA
ACAATATCTGATAGTCCTATGACAAACATAAATTATGGCACAGAATTCTCTCCTGTTGTGAATTCACA
AGAATATAAACCCAACCATGTCAAGCCAAATACAAGGCACAACCAATCAACATTCATCCACTGTCTTT
CCACTGCTACTTGGAGCAACTGAATTTCAGGACTCTGACCAGATGGGAAGAGGAAGAGAGCATTTCCA
AAGTAGACCCCCAATAACAGTAAGGACTATGATCAAAGATGTCAATGTCAAAATGCTTAGTAGCACCA
CCAACAAACTATTATTAGAGTCAGTAAATACCACAAATAGTCATCAGACATCTGTAAGAGAAGTGAGT
GAACCCAGGCACAATCACTTCTATTCTCACACTACTCAAATACTTAGCACCTCCACGTTCCCTTCAGA
TCCACACACAGCTGCTCATTCTCAGTTTCCGATCCCTAGAAATAGTACAGTTAACATCCCGCTGTTCA
GACGCTTTGGGAGGCAGAGGAAAATTGGCGGAAGGGGGCGGATTATCAGCCCATATAGAACTCCAGTT
CTGCGACGGCATAGATACAGCATTTTCAGGTCAACAACCAGAGGTTCTTCTGAAAAAAGCACTACTGC
ATTCTCAGCCACAGTGCTCAATGTGACATGTCTGTCCTGTCTTCCCAGGGAGAGGCTCACCACTGCCA
CAGCAGCATTGTCTTTTCCAAGTGCTGCTCCCATCACCTTCCCCAAAGCTGACATTGCTAGAGTCCCA
TCAGAAGAGTCTACAACTCTAGTCCAGAATCCACTATTACTACTTGAGAACAAACCCAGTGTAGAGAA
AACAACACCCACAATAAAATATTTCAGGACTGAAATTTCCCAAGTGACTCCAACTGGTGCAGTCATGA
CATATGCTCCAACATCCATACCCATGGAAAAAACTCACAAAGTAAACGCCAGTTACCCACGTGTGTCT
AGCACCAATGAAGCTAAAAGAGATTCAGTGATTACATCGTCACTTTCAGGTGCTATCACCAAGCCACC
AATGACTATTATAGCCATTACAAGGTTTTCAAGAAGGAAAATTCCCTGGCAACAGAACTTTGTAAATA
ACCATAACCCAAAAGGCAGATTAAGGAATCAACATAAAGTTAGTTTACAAAAAAGCACAGCTGTGATG
CTTCCTAAAACATCTCCTGCTTTACCACAGAGACAAAGTCTCCCCTCGCACCACACTACGACCAAAAC
ACACAATCCTGGAAGTCTTCCAACAAAGAAGGAGCTTCCCTTCCCACCCCTTAACCCTATGCTTCCTA
GTATTATAAGCAAAGACTCAAGTACAAAAAGCATCATATCAACGCAAACAGCAATACCAGCAACAACT
CCTACCTTCCCTGCATCTGTCATCACTTATGAAACCCAAACAGAGAGATCTAGAGCACAAACAATACA
AAGAGAACAGGAGCCTCAAAAGAAGAACAGGACTGACCCAAACATCTCTCCAGACCAGAGTTCTGGCT
TCACTACACCCACTGCTATGACACCTCCTGTTCTAACCACAGCCGAAACTTCAGTCAAGCCCAGTGTC
TCTGCATTCACTCATTCCCCACCAGAAAACACAACTGGGATTTCAAGCACAATCAGTTTTCATTCAAG
AACTCTTAATCTGACAGATGTGATTGAAGAACTAGCCCAAGCAAGTACTCAGACTTTGAAGAGCACAA
TTGCTTCTGAAACAACTTTGTCCAGCAAATCACACCAGAGTACCACAACTAGGAAAGCAATCATTAGA
CACTCAACCATACCACCATTCTTGAGCAGCAGTGCTACTCTAATGCCAGTTCCCATCTCCCCTCCCTT
TACTCAGAGAGCAGTTACTGACAACGTGGCGACTCCCATTTCCGGGCTTATGACAAATACAGTGGTCA
AGCTGCACGAATCCTCAAGGCACAATGCTAAACCACAGCAATTAGTAGCAGAGGTTGCAACATCCCCC
AAGGTTCACCCAAATGCCAAGTTCACAATTGGAACCACTCACTTCATCTACTCTAATCTGTTACATTC
TACTCCCATGCCAGCACTAACAACAGTTAAATCACAGAATTCTAAATTAACTCCATCTCCCTGGGCAG
AAAACCAATTTTGGCACAAACCATACTCAGAAATTGCTGAAAAAGGCAAAAAGCCAGAAGTAAGCATG
TTGGCTACTACAGGCCTGTCCGAGGCCACCACTCTTGTTTCAGATTGGGATGGACAGAAGAACACAAA
GAAGAGTGACTTTGATAAGAAACCAGTTCAAGAAGCAACAACTTCCAAACTCCTTCCCTTTGACTCTT
TGTCTAGGTATATATTTGAAAAGCCCAGGATAGTTGGAGGAAAAGCTGCAAGTTTTACTATTCCAGCT
AACTCAGATGCCTTTCTTCCCTGTGAAGCTGTTGGAAATCCCCTGCCCACCATTCATTGGACCAGAGT
CCCATCAGGTATGTCAGGACTTGATTTATCTAAGAGGAAACAGAATAGCAGGGTCCAGGTTCTCCCCA
ATGGTACCCTGTCCATCCAGAGGGTGGAAATTCAGGACCGCGGACAGTACTTGTGTTCCGCATCCAAT
CTGTTTGGCACAGACCACCTTCATGTCACCTTGTCTGTGGTTTCCTATCCTCCCAGGATCCTGGAGAG
ACGTACCAAAGAGATCACAGTTCATTCCGGAAGCACTGTGGAACTGAAGTGCAGAGCAGAAGGTAGGC
CAAGCCCTACAGTTACCTGGATTCTTGCAAACCAAACAGTTGTCTCAGAATCATCCCAGGGAACTAGG
CAGGCTGTGGTGACGGTTGACGGAACATTGGTCCTCCACAATCTCAGTATTTATGACCGTGGCTTTTA
CAAATGTGTGGCCAGCAACCCAGGTGGCCAGGATTCACTGCTGGTTAAAATACAAGTCATTGCAGCAC
CACCTGTTATTCTAGAGCAAAGGAGGCAAGTCATTGTAGGCACTTGGGGTGAAAGTTTAAAACTGCCC
ATTACAGTTTACCAATTCCAAGTTGTTCTTATTTTCAAATGGGACTTTGTATATAAGAAACCTAGCCT
CTTCAGACAGGGGCACTTATGAATGCATTGCTACCAGTTCCACTGGTTCGGAGCGAAGAGTAGTAATG
CTTACAATGGAAGAGCGAGTGACCAGCCCCAGGATAGAAGCTGCATCCCAGAAAAGGACTGAAGTGAA
TTTTGGGGACAAATTACTACTGAACTGCTCAGCCACTGGGGAGCCCAAACCCCAAATAATGTGGAGGT
TACCATCCAAGGCTGTGGTCGACCAGCAGCATAGGGTGGGCAGCTGGATCCACGTCTACCCTAATGGA
TCCCTGTTTATTGGATCAGTAACAGAAAAAGACAGTGGTGTCTACTTGTGTGTGGCAAGAAACAAAAT
ATTTTAGAAAGCAAGTGCTCCATGGGAAAGATTTCCAAGTAGATTGCAMAGCTTCCGGCTCCCCAGTG
CCAGAGATATCTTGGAGTTTGCCTGATGGAACCATGATCAACAATGCAATGCAAGCCGATGACAGTGG
CCACAGGACTAGGAGATATACCCTTTTCAACAATGGAACTTTATACTTCAACAAAGTTGGGGTAGCGG
AGGAAGGAGATTATACTTGCTATGCCCAGAACACCCTAGGGAAAGATGAAATGAAGGTCCACTTAACA
GTTATAACAGCTGCTCCCCGGATAAGGCAGAGTAACAAAACCAACAAGAGAATCAAAGCTGGAGACAC
AGCTGTCCTTGACTGTGAGGTCACTGGGGATCCCAAACCAAAAATATTTTGGTTGCTGCCTTCCAATG
ACATGATTTCCTTCTCCATTGATAGGTACACATTTCATGCCAATGGGTCTTTGACCATCAACAAAGTG
AAACTGCTCGATTCTGGAGAGTACGTATGTGTAGCCCGAAATCCCAGTGGGGATGACACCAAAATGTA
CAAACTGGATGTGGTCTCTAAACCTCCATTAATCAATGGTCTGTATACAAACAGAACTGTTATTAAAG
CCACAGCTGTGAGACATTCCAAAAAACACTTTGACTGCAGAGCTGAAGGGACACCATCTCCTGAAGTC
ATGTGGATCATGCCAGACAATATTTTCCTCACAGCCCCATACTATGGAAGCAGAATCACAGTCCATAA
AAATGGAACCTTGGAAATTAGGAATGTGAGGCTTTCAGATTCAGCCGACTTTATCTGTGTGGCCCGAA
ATGAAGGTGGAGAGAGCGTGTTGGTAGTACAGTTAGAAGTACTGGAAATGCTGAGAAGACCGACATTT
AGAAATCCATTTAATGAAAAAATAGTTGCCCAGCTGGGAAAGTCCACAGCATTGAATTGCTCTGTTGA
TGGTAACCCACCACCTGAAATAATCTGGATTTTACCAAATGGCACACGATTTTCCAATGGACCACAAA
GTTATCAGTATCTGATAGCAAGCAATGGTTCTTTTATCATTTCTAAAACAACTCGGGAGGATGCAGGA
AAATATCGCTGTGCAGCTAGGAATAAAGTTGGCTATATTGAGAAATTAGTCATATTAGAAATTGGCCA
GAAGCCAGTTATTCTTACCTATGCACCAGGGACAGTAAAAGGCATCAGTGGAGAATCTCTATCACTGC
ATTGTGTGTCTGATGGAATCCCTAAGCCAAATATCAAATGGACTATGCCAAGTGGTTATGTAGTAGAC
AGGCCTCAAATTAATGGGAAATACATATTGCATGACAATGGCACCTTAGTCATTAAAGAAGCAACAGC
TTATGACAGAGGAAACTATATCTGTAAGGCTCAAAATAGTGTTGGTCATACACTGATTACTGTTCCAG
TAATGATTGTAGCCTACCCTCCCCGAATTACAAATCGTCCACCCAGGAGTATTGTCACCAGGACAGGG
GCAGCCTTTCAGCTCCACTGTGTGGCCTTGGGAGTTCCCAAGCCAGAAATCACATGGGAGATGCCTGA
CCACTCCCTTCTCTCAACGGCAAGTAAAGAGAGGACACATGGAAGTGAGCAGCTTCACTTACAAGGTA
CCCTAGTCATTCAGAATCCCCAAACCTCCGATTCTGGGATATACAAATGCACAGCAAAGAACCCACTT
GGTAGTGATTATGCAGCAACGTATATTCAAGTAATCTGACATGAAATAATAAAGTC

| | | | |
|---|---|---|---|
| NOV29ak, SNP13376523 | SEQ ID NO:738 | 2617 aa | MW at 290181.1 kD |
| of CG55776-01, | SNP Pos: 61 | | SNP Change: |
| Protein Sequence | | | Arg to His |

MKVKGRGITCLLVSFAVICLVATPGGKACPRRCACYMPTEVHCTFRYLTSIPDSIPPNVEHINLGYNS
LVRLMETDFSGLTKLELLMLHSNGIHTIPDKTFSDLQALQVRLMVLKMSYNKVRKLQKDTFYGLRSLT
RLHMDHNNIEFINPEVFYGLNFLRLVHLEGNQLTKLHPDTFVSLSYLQIFKISFIKFLYLSDNFLTSL
PQEMVSYMPDLDSLYLHGNPWTCDCHLKWLSDWIQEKPGIYIVLPDVIKCKKDRSPSSAQQCPLCMNP
RTSKGKPLAMVSAAAFQCAKPTIDSSLKSKSLTILEDSSSAFISPQGFMAPFGSLTLNMTDQSGNEAN
MVCSIQKPSRTSPIAFTEENDYIVLNTSFSTFLVCNIDYGHIQPVWQILALYSDSPLILERSHLLSET
PQLYYKYKQVAPKPEDIFTNIEADLRADPSWLMQDQISLQLNRTATTFSTLQIQYSSDAQITLPRAEM
RPVKHKWTMISRDNNTKLEHTVLVGGTVGLNCPGQGDPTPHVDWLLADGSKVRAPYVSEDGRILIDKS
GKLELQMADSFDTGVYHCISSNYDDADILTYRITVVEPLVEAYQENGIHHTVFIGETLDLPCHSTGIP
DASISWVIPGNNVLYQSSRDKKVLNNGTLRILQVTPKDQGYYRCVAANPSGVDFLIFQVSVKMKGQRP
LEHDGETEGSGLDESNPIAHLKEPPGAQLRTSALMEAEVGKHTSSTSKRHNYRELTLQRRGDSTHRRF
RENRRHFPPSARRIDPQHWAALLEKAKKNAMPDKRENTTVSPPPVVTQLPNIPGEEDDSSGMLALHEE
FMVPATKALNLPARTVTADSRTISDSPMTNINYGTEFSPVVNSQILPPEEPTDFKLSTAIKTTAMSKN
INPTMSSQIQGTTNQHSSTVFPLLLGATEFQDSDQMGRGREHFQSRPPITVRTMIKDVNVKMLSSTTN
KLLLESVNTTNSHQTSVREVSEPRHNHFYSHTTQILSTSTFPSDPHTAAHSQFPIPRNSTVNIPLFRR
FGRQRKIGGRGRIISPYRTPVLRRHRYSIFRSTTRGSSEKSTTAFSATVLNVTCLSCLPRERLTTATA
ALSFPSAAPITFPKADIARVPSEESTTLVQNPLLLLENKPSVEKTTPTIKYFRTEISQVTPTGAVMTY
APTSIPMEKTHKVNASYPRVSSTNEAKRDSVITSSLSGAITKPPMTTIAITRFSRRKIPWQQNFVNNH
NPKGRLRNQHKVSLQKSTAVMLPKTSPALPQRQSLPSHHTTTKTHNPGSLPTKKELPFPPLNPMLPSI
ISKDSSTKSIISTQTAIPATTPTFPASVITYETQTERSRAQTIQREQEPQKKNRTDPNISPDQSSGFT
TPTAMTPPVLTTAETSVKPSVSAFTHSPPENTTGISSTISFHSRTLNLTDVIEELAQASTQTLKSTIA
SETTLSSKSHQSTTTRKAIIRHSTIPPFLSSSATLMPVPISPPFTQRAVTDNVATPISGLMTNTVVKL
HESSRHNAKPQQLVAEVATSPKVHPNAKFTIGTTHFIYSNLLHSTPMPALTTVKSQNSKLTPSPWAEN
QFWHKPYSEIAEKGKKPEVSMLATTGLSEATTLVSDWDGQKNTKKSDFDKKPVQEATTSKLLPFDSLS
PTVTWILANQTVVSESSQGSRQAVVTVDGTLVLHNLSIYDRGFYKCVASNPGGQDSLLVKIQVIAAPP
VILEQRRQVIVGTWGESLKLPCTAKGTPQPSVYWVLSDGTEVKPLQFTNSKLFLFSNGTLYIRNLASS
DRGTYECIATSSTGSERRVVMLTMEERVTSPRIEAASQKRTEVNFGDKLLLNCSATGEPKPQIMWRLP
SKAVVDQQHRVGSWIHVYPNGSLFIGSVTEKDSGVYLCVARNKMGDDLILMHVSLRLKPAKIDHKQYF
RKQVLHGKDFQVDCKASGSPVPEISWSLPDGTMINNAMQADDSGHRTRRYTLFNNGTLYFNKVGVAEE
GDYTCYAQNTLGKDEMKVHLTVITAAPRIRQSNKTNKRIKAGDTAVLDCEVTGDPKPKIFWLLPSNDM
ISFSIDRYTFHANGSLTINKVKLLDSGEYVCVARNPSGDDTKMYKLDVVSKPPLINGLYTNRTVIKAT
AVRHSKKHFDCRAEGTPSPEVMWIMPDNIFLTAPYYGSRITVHKNGTLEIPNVRLSDSADFICVARNE
GGESVLVVQLEVLEMLARRPTFAPFNEKIVAQLGKSTAIACSVDGNPPPEIIWILPNGTRFSNGPQSY
QYLIASNGSFIISKTTREDAGKYRCAARNKVGYIEKLVILEIGQKPVILTYAPGTVKGISGESLSLHC
VSDGIPKPNIKWTMPSGYVVDRPQINGKYILHDNGTLVIKEATAYDRGNYICKAQNSVGHTLITVPVM
IVAYPPRITNRPPRSIVTRTGAAFQLHCVALGVPKPEITWEMPDHSLLSTASKERTHGSEQLHLQGTL
VIQNPQTSDSGIYKCTAKNPLGSDYAATYIQVI

| | | |
|---|---|---|
| NOV29al, SNP13376524 of | SEQ ID NO:739 | 7876 bP |
| CG55776-01, | ORF Start: ATG at 6 | ORF Stop: TGA at 7857 |
| DNA Sequence | SNP Pos: 251 | SNP Change: A to T |

TCAGGATGAAGGTAAAAGGCAGAGGAATCACCTGCTTGCTGGTCTCCTTTGCTGTGATCTGCCTGGTC
GCCACCCCTGGGGGCAAGGCCTGTCCTCGCCGCTGTGCCTGTTATATGCCTACGGAGGTACACTGCAC
ATTTCGGTACCTGACTTCCATCCCAGACAGCATCCCGCCCAATGTGGAACGCATCAATTTAGGGTACA
ACAGCTTGGTTAGATTGATGGAAACAGATTTTTCTGGCCTGACCAATCTGGAGTTACTCATGCTTCAC
AGCAATGGCATTCACACAATCCCTGACAAGACCTTCTCAGATTTGCAGGCCTTGCAGGTGAGACTGAT
GGTCTTAAAAATGAGCTATAATAAAGTCCGAAAACTTCAGAAAGATACTTTTTATGGCCTCAGGAGCT
TGACACGATTGCACATGGACCACAACAATATTGAGTTTATAAACCCAGAGGTTTTTTATGGGCTCAAC
TTTCTCCGCCTGGTGCACTTGGAAGGAAATCAGCTCACTAAGCTCCACCCAGATACATTTGTCTCTTT
GAGCTACCTCCAGATATTTAAAATCTCTTTCATTAAGTTCCTATACTTGTCTGATAACTTCCTGACCT
CCCTCCCTCAAGAGATGGTCTCCTATATGCCTGACCTAGACAGCCTTTACCTGCATGGAAACCCATGG
ACCTGTGATTGCCATTTAAAGTGGTTGTCTGACTGGATACAGGAGAAGCCAGGTATCTATATTGTNTT
ACCAGATGTAATAAAATGCAAAAAAGATAGAAGTCCCTCTAGTGCTCAGCAGTGTCCACTTTGCATGA
ACCCTAGGACTTCTAAAGGCAAGCCGTTAGCTATGGTCTCAGCTGCAGCTTTCCAGTGTGCCAAGCCA
ACCATTGACTCATCCCTGAAATCAAAGAGCCTGACTATTCTGGAAGACAGTAGTTCTGCTTTCATCTC
TCCCCAAGGTTTCATGGCACCCTTTGGCTCCCTCACTTTGAATATGACAGATCAGTCTGGAAATGAAG
CTAACATGGTCTGCAGTATTCAAAAGCCCTCAAGGACATCACCCATTGCATTCACTGAAGAAAATGAC
TACATCGTGCTAAATACTTCATTTTCAACATTTTTGGTGTGCAACATAGATTACGGTCACATTCAGCC
AGTGTGGCAAATTTTGGCTTTGTACAGTGATTCTCCTCTGATACTAGAAAGGAGCCACTTGCTTAGTG
AAACACCGCAGCTCTATTACAAATATAAACAGGTGGCTCCTAAGCCTGAAGACATTTTTACCAACATA
GAGGCAGATCTCAGAGCAGATCCCTCTTGGTTAATGCAAGACCAAATTTCCTTGCAGCTGAACAGAAC
TGCCACCACATTCAGTACATTACAGATCCAGTACTCCAGTGATGCTCAAATCACTTTACCAAGAGCAG
AGATGAGGCCAGTGAAAACACAAATGGACTATGATTTCAAGGGATACAATACTAAGCTGGAACATACT
GTCTTGGTAGGTGGAACCGTTGGCCTGAACTGCCCAGGCCAGGAGACCCCACCCCACAACGTGGATTG
GCTTCTAGCTGATGGAAGTAAAGTGAGAGCCCCTTATGTCAGTGAGGATGGACGGATCCTAATAGACA
AATTATGATGATGCAGATATTCTCACCTATAGGATAACTGTGGTAGAACCTTTGGTCGAAGCCTATCA
GGAAAATGGGATTCATCACACAGTTTTCATTGGTGAAACACTTGATCTTCCATGCCATTCTACTGGTA
TCCCAGATGCCTCTATTAGCTGGGTTATTCCAGGAAACAATGTGCTCTATCAGTCATCAAGAGACAAG
TGTGGCAGCCAACCCATCAGGGGTTGATTTTTTGATTTTCCAAGTTTCAGTCAAGATGAAAGGACAAA
GGCCCTTGGAGCATGATGGAGAAACAGAGGGATCTGGACTTGATGAGTCCAATCCTATTGCTCATCTT
AAGGAGCCACCAGGTGCACAACTCCGTACATCTGCTCTGATGGAGGCTGAGGTTGGAAAACACACCTC
AGCACAGTAAGAGGCACA1ACTATCGGGAAATTAACACTCCAGCGACGTGGAGATTCAACACATCGAC
GTTTTAGGGAGAATAGGAGGCATTTCCCTCCCTCTGCTAGGAGAATTGACCCACAACATTGGGCGGCA
CTGTTGGAGAAAGCTAAAAAGAATGCTATGCCAGACAAGCGAGAAAATACCACAGTGAGCCCACCCCC
AGTGGTCACCCAACTCCCAAACATACCTGGTGAAGAAGACGATTCCTCAGGCATGCTCGCTCTACATG
AGGAATTTATGGTCCCGGCCACTAAAGCTTTGAACCTTCCAGCAAGGACAGTGACTGCTGACTCCAGA
ACAATATCTGATAGTCCTATGACAAACATAAATTATGGCACAGAATTCTCTCCTGTTGTGAATTCACA
AATACTACCACCTGAAGAACCCACAGATTTCAAACTGTCTACTGCTATTAAAACTACAGCCATGTCAA
AGAATATAAACCCAACCATGTCAAGCCAAATACAAGGCACAACCAATCAACATTCATCCACTGTCTTT
AAGTAGACCCCCAATAACAGTAAGGACTATGATCAAAGATGTCAATGTCAAAATGCTTAGTAGCACCA
CCAACAAACTATTATTAGAGTCAGTAAATACCACAAATAGTCATCAGACATCTGTAAGAGAAGTGAGT
GAACCCAGGCACAATCACTTCTATTCTCACACTACTCAAATACTTAGCACCTCCACGTTCCCTTCAGA
TCCACACACAGCTGCTCATTCTCAGTTTCCGATCCCTAGAAATAGTACAGTTAACATCCCGCTGTTCA
GACGCTTTGGGAGGCAGAGGAAAATTGGCGGAAGGGGGCGGATTATCAGCCCATATAGAACTCCAGTT
CTGCGACGGCATAGATACAGCATTTTCAGGTCAACAACCAGAGGTTCTTCTGAAAAAAGCACTACTGC
ATTCTCAGCCACAGTGCTCAATGTGACATGTCTGTCCTGTCTTCCCAGGGAGAGGCTCACCACTGCCA
CAGCAGCATTGTCTTTTCCAAGTGCTGCTCCCATCACCTTCCCCAAAGCTGACATTGCTAGAGTCCCA
TCAGAAGAGTCTACAACTCTAGTCCAGAATCCACTATTACTACTTGAGAACAAACCCAGTGTAGAGAA
AACAACACCCACAATAAATATTTCAGGACTGAAATTTCCCAAAGTGACTCCAACTGGTGCAGTCATGA
CATATGCTCCAAACATCCATACCCATGGAAACTCACAAAGTAAACGCCAAGTTACCCAACGTGTGTCT
AGCACCAATGAAGCTAAAAGAGATTCAGTGATTACATCGTCACTTTCAGGTGCTATCACCAAGCCACC
AATGACTATTATAGCCATTACAAGGTTTTCAAGAAGGAAAATTCCCTGGCAACAGAACTTTGTAAATA
ACCATAACCCAAAAGGCAGATTAAGGAATCAACATAAAGTTAGTTTACAAAAAAGCACAGCTGTGATG
CTTCCTAAAACATCTCCTGCTTTACCACAGAGACAAAGTCTCCCCTCGCACCACACTACGACCAAAAC
ACACAATCCTGGAAGTCTTCCAACAAAGAAGGAGCTTCCCTTCCCACCCCTTAACCCTATGCTTCCTA
GTATTATAAGCAAAGACTCAAGTACAAAAGCATCAATATCAACGCAAACAGCAATACCAGCAACAACT
CCTACCTTCCCTGCATCTGTCATCACTTATGACCCAAACAAGAGAGATCTAGAGCACAAAACAATACA
AAGAGAACAGGAGCCTCAAAGAAAGAAkCAGGACTGACCCAACATCTCTCCAGACCAGAGTTCTGGCT
TCACTACACCCACTGCTATGACACCTCCTGTTCTAACCACAGCCGAAACTTCAGTCAAGCCCAGTGTC
TCTGCATTCACTCATTCCCCACCAGAACACAAACTGGGAATTTCAAGCACAATCAGTTTTCATTCAAG
AACTCTTAATCTGACAGATGTGATTGAAGAACTAGCCCAGCAAAGTACTCAGACTTTGAAGAGCACAA
TTGCTTCTGAAACAACTTTGTCCAGCAAATCACACCAGAGTACCACAACTAGGAAAGCAATCATTAGA
CACTCAACCATACCACCATTCTTGAGCAGCAGTGCTACTCTAATGCCAGTTCCCATCTCCCCTCCCTT
TACTCAGAGAGCAGTTACTGACAACGTGGCGACTCCCATTTCCGGGCTTATGACAAATACAGTGGTCA
AGCTGCACGAATCCTCAAGGCACAATGCTAAACCACAGCAATTAGTAGCAGAGGTTGCAACATCCCCC
AAGGTTCACCCAAATGCCAAGTTCACAATTGGAACCACTCACTTCATCTACTCTAATCTGTTACATTC
TACTCCCATGCCAGCACTAACAACAGTTAAATCACAGAATTCTAAATTAACTCCATCTCCCTGGGCAG
AAAACCAATTTTGGCACAAACCATACTCAGAAATTGCTGAAAAAGGCAAAAAGCCAGAAGTAAGCATG
TTGGCTACTACAGGCCTGTCCGAGGCCACCACTCTTGTTTCAGATTGGGATGGACAGAAGAACACAAA
GAAGAGTGACTTTGATAAGAAACCAGTTCAAGAAGCAACAACTTCCAAACTCCTTCCCTTTGACTCTT
TGTCTAGGTATATATTTGAAAAGCCCAGGATAGTTGGAGGAAAAGCTGCAAGTTTTACTATTCCAGCT
AACTCAGATGCCTTTCTTCCCTGTGAAGCTGTTGGAAATCCCCTGCCCACCATTCATTGGACCAGAGT
CCCATCAGGTATGTCAGGACTTGATTTATCTAAGAGGAAACAGAATAGCAGGGTCCAGGTTCTCCCCA
ATGGTACCCTGTCCATCCAGAGGGTGGAAATTCAGGACCGCGGACAGTACTTGTGTTCCGCATCCAAT
CTGTTTGGCACAGACCACCTTCATGTCACCTTGTCTGTGGTTTCCTATCCTCCCAGGATCCTGGAGAG
ACGTACCAAGAGATCACAGTTCATTCCGGAAGCACTGTGGAACTGAAGTGCAGAGCAGAAGGTAAGGC
CAAGCCCTACAGTTACCTGGATTCTTGCAAACCAAACAGTTGTCTCAGAATCATCCCAGGGAAGTAGG
CAGGCTGTGGTGACGGTTGACGGAACATTGGTCCTCCACAATCTCAGTATTTATGACCGTGGCTTTTA
CAAATGTGTGGCCAGCAACCCAGGTGGCCAGGATTCACTGCTGGTTAAAATACAAGTCATTGCAGCAC
CACCTGTTATTCTAGAGCATAGGAGGCAAGTCATTGTAGGCACTTGGGGTGAAAGTTTAAAACTGCCC
TGTACTGCAAAAGGAACTCCTCAGCCCAGCGTTTACTGGGTCCTCTCTGATGGCACTGAAGTGAAACC
ATTACAGTTTACCAATTCCAAGTTGTTCTTATTTTCAAATGGGACTTTGTATATAAGAAACCTAGCCT
CTTCAGACAGGGGCACTTATGAATGCATTGCTACCAGTTCCACTGGTTCGGAGCGAAGAGTAGTAATG
CTTACAATGGAAGAGCGAGTGACCAGCCCCAGGATAGAAGCTGCATCCCAGAAAAGGACTGAAGTGAA
TTTTGGGGACAAATTACTACTGAACTGCTCAGCCACTGGGGAGCCCAAACCCCAAATAATGTGGAGGT
GGGGGATGATCTGATACTGATGCATGTTAGCCTAAGACTGAAACCTGCCAAAATTGACCACAAGCAGT
ATTTTAGAAAGCAAGTGCTCCATGGGAAAGATTTCCAAGTAGATTGCAAAGCTTCCGGCTCCCCAGTG
CCAGAGATATCTTGGAGTTTGCCTGATGGAACCATGATCAACAATGCAATGCAAGCCGATGACAGTGG
CCACAGGACTAGGAGATATACCCTTTTCAACAATGGAACTTTATACTTCAACAAAGTTGGGGTAGCGG
AGGAAGGAGATTATACTTGCTATGCCCAGAACACCCTAGGGAAAAGATGAATGAAGGTCCACTTAACA
GTTATAACAGCTGCTCCCCGGATAAGGCAGAGTAACAAAACCAACAAGAGAATCAAAGCTGGAGACAC
AGCTGTCCTTGACTGTGAGGTCACTGGGGATCCCAAACCAAAAATATTTTGGTTGCTGCCTTCCAATG
AAACTGCTCGATTCTGGAGAGTACGTATGTGTAGCCCGAAATCCCAGTGGGGATGACACCAAAATGTA
CAAACTGGATGTGGTCTCTAAACCTCCATTAATCAATGGTCTGTATACAAACAGAACTGTTATTAAAG
CCACAGCTGTGAGACATTCCAAAAAACACTTTGACTGCAGAGCTGAAGGGACACCATCTCCTGAAGTC
ATGTGGATCATGCCAGACAATATTTTCCTCACAGCCCCATACTATGGAAGCAGAATCACAGTCCATAA
AAATGGAACCTTGGAAATTAGGAATGTGAGGCTTTCAGATTCAGCCGACTTTATCTGTGTGGCCCGAA
ATGAAGGTGGAGAGAGCGTGTTGGTAGTACAGTTAGAAGTACTGGAAATGCTGAGAAGACCGACATTT
AGAAATCCATTTAATGAAAAAATAGTTGCCCAGCTGGGAAAGTCCACAGCATTGAATTGCTCTGTTGA
TGGTAACCCACCACCTGAAATAATCTGGATTTTACCAAATGGCACACGATTTTCCAATGGACCACAAA
GTTATCAGTATCTGATAGCAAGCAATGGTTCTTTTATCATTTCTAAAACAACTCGGGAGGATGCAGGA
AAATATCGCTGTGCAGCTAGGAATAA)GTTGGCTATATTGAGAAATTAGTCATATTAGAAATTGGCCA
GAAGCCAGTTATTCTTACCTATGCACCAGGGACAGTAAAAGGCATCAGTGGAGAATCTCTATCACTGC
ATTGTGTGTCTGATGGAATCCCTAAGCCAAATATCAAATGGACTATGCCAAGTGGTTATGTAGTAGAC
AGGCCTCAAATTAATGGGAAATACATATTGCATGACAATGGCACCTTAGTCATTAAAGAAGCAACAGC
TTATGACAGAGGAAACTATATCTGTAAGGCTCAAAATAGTGTTGGTCATACACTGATTACTGTTCCAG
TAATATGATTGTAGCCTACCCTCCCCG1ATTACATCGTCCACCCAGGAGTATTGTCACCAGGACAGGG
GCAGCCTTTCAGCTCCACTGTGTGGCCTTGGGAGTTCCCAAGCCAGAAATCACATGGGAGATGCCTGA
CCCTAGTCATTCAGAATCCCCAAACCTCCGATTCTGGGATATACAAATGCACAGCAAAGAACCCACTT
GGTAGTGATTATGCAGCAACGTATATTCAAGTAATCTGACATGAAATAATAAAGTC

| | | | |
|---|---|---|---|
| NOV29al, SNP13376524 of | SEQ ID NO:740 | 2617 aa | MW at 290186.1 kD |
| CG55776-01, Protein Sequence | SNP Pos: 82 | | ISNP Change: |
| | | | LYS to ASn |

MKVKGRGITCLLVSFAVICLVATPGGKACPRRCACYMPTEVHCTFRYLTSIPDSIPPNVERINLGYNS
LVRLMETDFSGLTNLELLMLHSNGIHTIPDKTFSDLQALQVRIMVLKMSYNKVRKLQKDTFYGLRSLT
RLHMDHNNIEFINPEVFYGLNFLRLVHLEGNQLTKLHPDTFVSLSYLQIFKISFIKFLYLSDNFLTSL
PQEMVSYMPDLDSLYLHGNPWTCDCHLKWLSDWIQEKPGIYIVLPDVIKCKKDRSPSSAQQCPLCMNP
RTSKGKPLAMVSAAAFQCAKPTIDSSLKSKSLTILEDSSSAFISPQGFMAPFGSLTLNMTDQSGNEAN
MVCSIQKPSRTSPIAFTEENDYIVLNTSFSTFLVCNIDYGHIQPVWQILALYSDSPLILERSHLLSET
PQLYYKYKQVAPKPEDIFTNIEADLRADPSWLMQDQISLQLNRTATTFSTLQIQYSSDAQITLPRAEM
RPVKHKWTMISRDNNTKLEHTVLVGGTVGLNCPGQGDPTPHVDWLLADGSKVPAPYVSEDGRILIDKS
GKLELQMADSFDTGVYHCISSNYDDADILTYRITVVEPLVEAYQENGIHHTVFIGETLDLPCHSTGIP
DASISWVIPGNNVLYQSSRDKKVLNNGTLRILQVTPKDQGYYRCVAANPSGVDFLIFQVSVKMKGQRP
LEHDGETEGSGLDESNPIAHLKEPPGAQLRTSALMEAEVGKHTSSTSKRHNYRELTLQRRGDSTHRRF
RENRRHFPPSARRIDPQHWAALLEKAKKNAMPDKRENTNTSPPPVVTQLPNIPGEEDDSSGMLALHEE
FMVPATKALNLPARTVTADSRTISDSPMTNTNYGTEFSPVVNSQILPPEEPTDFKLSTAIKTTAMSKN
INPTMSSQIQGTTNQHSSTVFPLLLGATEFQDSDQMGRGREHFQSRPPITVRTMIKDVNVKMLSSTTN
KLLLESVNTTNSHQTSVREVSEPRHNHFYSHTTQILSTSTFPSDPHTAAHSQFPIPPNSTVNIPLFRR
FGRQRKIGGRGRIISPYRTPKTLRHRYSIFRSTTRGSSEKSTTAFSATVLNVTCLSCLPRERLTTATA
ALSFPSAAPITFPKADIARVPSEESTTLVQNPLLLLENKPSVEKTTPTIKYFRTEISQVTPTGAVMTY
APTSIPMEKTHKVNASYPRVSSTNEAKRDSVITSSLSGAITKPPMTIIAITRFSRRKIPWQQNFVNNH
NPKGRLRNQHKVSLQKSTAVMLPKTSPALPQRQSLPSHHTTTKTHNPGSLPTKKELPFPPLNPMLPSI
ISKDSSTKSIISTQTAIPATTPTFPASVITYETQTERSRAQTIQREQEPQKKNRTDPNISPDQSSGFT
TPTAMTPPVLTTAETSVKPSVSAFTHSPPENTTGISSTISFHSRTLNLTDVIEELAQASTQTLKSTIA
SETTLSSKSHQSTTTRKAIIRHSTIPPFLSSSATLMPVPISPPFTQRAVTDNVATPISGLMTNTVVKL
HESSRHNAKPQQLVAEVATSPKVHPNAKFTIGTTHFIYSNLLHSTPMPALTTVKSQNSKLTPSPWAEN
QFWHKPYSEIAEKGKKPEVSMLATTGLSEATTLVSDWDGQKATKKSDFDKKPVQEATTSKLLPFDSLS
RYIFEKPRIVGGKAASFTIPANSDAFLPCEAVGNPLPTIHWTRVPSGMSGLDLSKRKQNSRVQVLPNG
TLSIQRVEIQDRGQYLCSASNLFGTDHLHVTLSVVSYPPRILERRTKEITVHSGSTVELKCRAEGRPS
PTVTWILANQTVVSESSQGSRQAVVTVDGTLVLHNLSIYDRGFYKCVASNPGGQDSLLVKIQVIAAPP
VILEQRRQVIVGTWGESLKLPCTAKGTPQPSVYWVLSDGTEVKPLQFTNSKLFLFSNGTLYIRNLASS
DRGTYECIATSSTGSERRVVMLTMEERVTSPRIEAASQKRTEVNFGDKLLLNCSATGEPKPQIMWRLP
SKAVVDQQHRVGSWIHVYPNGSLFIGSVTEKDSGVYLCVARNKMGDDLILMHVSLRLKPAKIDHKQYF
RKQVLHGKDFQVDCKASGSPVPEISWSLPDGTMINNAMQADDSGHRTRRYTLFNNGTLYFNKVGVAEE
GDYTCYAQNTLGKDENKVHLTVITAAPRIRQSNKTAKRIKAGDTAVLDCEVTGDPKPKIFWLLPSNDM
ISFSIDRYTFHANGSLTINKVKLLDSGEYVCVARNPSGDDTKMYKLDVVSKPPLINGLYTNRTVIKAT
AVRHSKKHFDCRAEGTPSPEVMWIMPDNIFLTAPYYGSRITVHKAGTLEIRNVRLSDSADFICVARNE
GGESVLVVQLEVLEMLRRPTFRNPFNEKIVAQLGKSTALNCSVDGNPPPEIIWILPNGTRFSNGPQSY
QYLIASNGSFIISKTTREDAGKYRCAARNKVGYIEKLVILEIGQKPVILTYAPGTVKGISGESLSLHC
VSDGIPKPNIKWTMPSGYVVDRPQINGKYILHDNGTLVIKEATAYDRGNYICKAQNSVGHTLITVPVM
IVAYPPRITNRPPRSIVTRTGAAFQLHCVALGVPKPEITWEMPDHSLLSTASKERTHGSEQLHLQGTL
VIQNPQTSDSGIYKCTAKNPLGSDYAATYIQVI

| | | |
|---|---|---|
| NOV29am, SNP13376525 of | SEQ ID NO:741 | 7876 bP |
| CG55776-01, | ORF Start: ATG at 6 | ORF Stop: TGA at 7857 |
| DNA Sequence | SNP Pos: 493 | SNP Change: A to G |

TCAGGATGAAGGTAAAAGGCAGAGGAATCACCTGCTTGCTGGTCTCCTTTGCTGTGATCTGCCTGGTC
GCCACCCCTGGGGGCAAGGCCTGTCCTCGCCGCTGTGCCTGTTATATGCCTACGGAGGTACACTGCAC
ATTTCGGTACCTGACTTCCATCCCAGACAGCATCCCGCCCAATGTGGAACGCATCAATTTAGGGTACA
ACAGCTTGGTTAGATTGATGGAAACAGATTTTTCTGGCCTGACCAAACTGGAGTTACTCATGCTTCAC
AGCAATGGCATTCACACAATCCCTGACAAGACCTTCTCAGATTTGCAGGCCTTGCAGGTGAGACTGAT
GGTCTTAAAAATGAGCTATAATAAAGTCCGAAAACTTCAGAAAGATACTTTTTATGGCCTCAGGAGCT
TGACACGATTGCACATGGACCACAACAATATTGAGTTTATAAACCCAGAGGTTTTTTATGGGCTCAAC
TTTCTCCGCCTGGTGCGCTTGGAAGGAAATCAGCTCACTAAGCTCCACCCAGATACATTTGTCTCTTT
GAGCTACCTCCAGATCTTTAAAATCTCTTTCATTAAGTTCCTATACTTGTCTGATAACTTCCTGACCT
CCCTCCCTCAAGAGATGGTCTCCTATATGCCTGACCTAGACAGCCTTTACCTGCATGGAAACCCATGG
ACCTGTGATTGCCATTTAAAGTGGTTGTCTGACTGGATACAGGAGAAGCCAGGTATCTATATTGTNTT
ACCAGATGTAATAAATGCAA3AAAAGATAGAAGTCCCTCTAGTGCTCAGCAGTGTCCACTTTGCATGA
ACCCTAGGACTTCTAAAGGCAAGCCGTTAGCTATGGTCTCAGCTGCAGCTTTCCAGTGTGCCAAGCCA
ACCATTGACTCATCCCTGAAATCAAAGAGCCTGACTATTCTGGAAGACAGTAGTTCTGCTTTCATCTC
TCCCCAAGGTTTCATGGCACCCTTTGGCTCCCTCACTTTGAATATGACAGATCAGTCTGGAAATGAAG
CTAACATGGTCTGCAGTATTCAAAAGCCCTCAAGGACATCACCCATTGCATTCACTGAAGAAAATGAC
TACATCGTGCTAAATACTTCATTTTCAACATTTTTGGTGTGCAACATAGATTACGGTCACATTCAGCC
AGTGTGGCAAATTTTGGCTTTGTACAGTGATTCTCCTCTGATACTAGAAAGGAGCCACTTGCTTAGTG
AAACACCGCAGCTCTATTACAAATATAAACAGGTGGCTCCTAAGCCTGAAGACATTTTTACCAACATA
GAGGCAGATCTCAGAGCAGATCCCTCTTGGTTAATGCAAGACCAAATTTCCTTGCAGCTGAACAGAAC
TGCCACCACATTCAGTACATTACAGATCCAGTACTCCAGTGATGCTCAAATCACTTTACCAAGAGCAG
AGATGAGGCCAGTGAAACACAAATGGACTATGATTTCAAGGGATAACAATACTAAGCTGGAACATACT
GTCTTGGTAGGTGGAACCGTTGGCCTGAACTGCCCAGGCCAAGGAGACCCCACCCCACACGTGGATTG
GCTTCTAGCTGATGGAAGTAAAGTGAGAGCCCCTTATGTCAGTGAGGATGGACGGATCCTAATAGACA
AAAGTGGAAAATTGGAACTCCAGATGGCTGATAGTTTTGACACAGGCGTATATCACTGTATAAGCAGC
AATTATGATGATGCAGATATTCTCACCTATAGGATAACTGTGGTAGAACCTTTGGTCGAAGCCTATCA
GGAAAATGGGATTCATCACACAGTTTTCATTGGTGAAACACTTGATCTTCCATGCCATTCTACTGGTA
TCCCAGATGCCTCTATTAGCTGGGTTATTCCAGGAAACAATGTGCTCTATCAGTCATCAAGAGACAAG
AAAGTTCTAAACAATGGCACATTAAGAATATTACAGGTCACCCCGAAAGACCAAGGTTATTATCGCTG
TGTGGCAGCCAACCCATCAGGGGTTGATTTTTTGATTTTCCAAGTTTCAGTCAAGATGAAAGGACAAA
GGCCCTTGGAGCATGATGGAGAAACAGAGGGATCTGGACTTGATGAGTCCAATCCTATTGCTCATCTT
AAGGAGCCACCAGGTGCACAACTCCGTACATCTGCTCTGATGGAGGCTGAGGTTGGAAAACACACCTC
AAGCACkAGTAAGAGGCACAACTATCGGGAATTAACACTCCAGCGACGTGGAGATTCAACACATCGAC
GTTTTAGGGAGAATAGGAGGCATTTCCCTCCCTCTGCTAGGAGAATTGACCCACAACATTGGGCGGCA
CTGTTGGAGAAAGCTAAAAAGAATGCTATGCCAGACAAGCGAGAAAATACCACAGTGAGCCCACCCCC
AGTGGTCACCCAACTCCCAAACATACCTGGTGAAGAAGACGATTCCTCAGGCATGCTCGCTCTACATG
AGGAATTTATGGTCCCGGCCACTAAAGCTTTGAACCTTCCAGCAAGGACAGTGACTGCTGACTCCAGA
ACAATATCTGATAGTCCTATGACAAACATAAATTATGGCACAGAATTCTCTCCTGTTGTGAATTCACA
AATACTACCACCTGAAGAACCCACAGATTTCAAACTGTCTACTGCTATTAAAACTACAGCCATGTCAA
AGAATATAAACCCAACCATGTCAAGCCAAATACAAGGCACAACCAATCAACATTCATCCACTGTCTTT
CCACTGCTACTTGGAGCAACTGAATTTCAGGACTCTGACCAGATGGGAAGAGGAAGAGAGCATTTCCA
AAGTAGACCCCCAATAACAGTAAGGACTATGATCAAAGATGTCAATGTCAAAATGCTTAGTAGCACCA
CCAACAAACTATTATTAGAGTCAGTAAATACCACAAATAGTCATCAGACATCTGTAAGAGAAGTGAGT
GAACCCAGGCACAATCACTTCTATTCTCACACTACTCAAATACTTAGCACCTCCACGTTCCCTTCAGA
TCCACACACAGCTGCTCATTCTCAGTTTCCGATCCCTAGAAATAGTACAGTTAACATCCCGCTGTTCA
GACGCTTTGGGAGGCAGAGGAAAATTGGCGGAAGGGGGCGGATTATCAGCCCATATAGAACTCCAGTT
CTGCGACGGCATAGATACAGCATTTTCAGGTCAACAACCAGAGGTTCTTCTGAAAAAAGCACTACTGC
ATTCTCAGCCACAGTGCTCAATGTGACATGTCTGTCCTGTCTTCCCAGGGAGAGGCTCACCACTGCCA
CAGCAGCATTGTCTTTTCCAAGTGCTGCTCCCATCACCTTCCCCAAAGCTGACATTGCTAGAGTCCCA
TCAGAAGAGTCTACAACTCTAGTCCAGAATCCACTATTACTACTTGAGAACAAACCCAGTGTAGAGAA
AACAACACCCACAATAAAATATTTCAGGACTGAAATTTCCCAAGTGACTCCAACTGGTGCAGTCATGA
CATATGCTCCAACATCCATACCCATGGAAIAAACTCACAAAGTAAACGCCAGTTACCCACGTGTGTCT
AATGACTATTATAGCCATTACAAGGTTTTCAAGAAGGAAAATTCCCTGGCAACAGAACTTTGTAAATA
ACCATAACCCAAAAGGCAGATTAAGGAATCAACATAAAGTTAGTTTACAAAAAAGCACAGCTGTGATG
CTTCCTAAAACATCTCCTGCTTTACCACAGAGACAAAGTCTCCCCTCGCACCACACTACGACCAAAAC
ACACAATCCTGGAAGTCTTCCAACAAAGAAGGAGCTTCCCTTCCCACCCCTTAACCCTATGCTTCCTA
GTATTATAAGCAAAGACTCAAGTAACAAAGCATCAATATCAACGCAAACAGCAATACCAGCAACAACT
CCTACCTTCCCTGCATCTGTCATCACTTATGAAACCCAAACAGAGAGATCTAGAGCACAAACAATACA
AAGAGAACAGGAGCCTCAAAAGAAGAACAGGACTGACCCAAACATCTCTCCAGACCAGAGTTCTGGCT
TCACTACACCCACTGCTATGACACCTCCTGTTCTAACCACAGCCGAAACTTCAGTCAAGCCCAGTGTC
TCTGCATTCACTCAATTCCCCACCAGAAACACAACTGGGATTTCAAGCACAATCAGTTTTCATTCAAG
TTGCTTCTGAAACAACTTTGTCCAGCAAATCACACCAGAGTACCACAACTAGGAAAGCAATCATTAGA
CACTCAACCATACCACCATTCTTGAGCAGCAGTGCTACTCTAATGCCAGTTCCCATCTCCCCTCCCTT
TACTCAGAGAGCAGTTACTGACAACGTGGCGACTCCCATTTCCGGGCTTATGACAAATACAGTGGTCA
AGCTGCACGAATCCTCAAGGCACAATGCTAAACCACAGCAATTAGTAGCAGAGGTTGCAACATCCCCC
AAGGTTCACCCAAATGCCAAGTTCACAATTGGAACCACTCACTTCATCTACTCTAATCTGTTACATTC
TACTCCCATGCCAGCACTAACAACAGTTAAATCACAGAATTCTAAATTAACTCCATCTCCCTGGGCAG
AAAACCAATTTTGGCACAAACCATACTCAGAAATTGCTGAAAAAGGCAAAAAGCCAGAAGTAAGCATG
TTGGCTACTACAGGCCTGTCCGAGGCCACCACTCTTGTTTCAGATTGGGATGGACAGAAGAACACAAA
GAAGAGTGACTTTGATAAGAAACCAGTTCAAGAAGCAACAACTTCCAAACTCCTTCCCTTTGACTCTT
TGTCTAGGTATATATTTGAAAAGCCCAGGATAGTTGGAGGAAAAGCTGCAAGTTTTACTATTCCAGCT
AACTCAGATGCCTTTCTTCCCTGTGAAGCTGTTGGAAATCCCCTGCCCACCATTCATTGGACCAGAGT
CCCATCAGGTATGTCAGGACTTGATTTATCTAAGAGGAAACAGAATAGCAGGGTCCAGGTTCTCCCCA
ATGGTACCCTGTCCATCCAGAGGGTGGAAATTCAGGACCGCGGACAGTACTTGTGTTCCGCATCCAAT
CTGTTTGGCACAGACCACCTTCATGTCACCTTGTCTGTGGTTTCCTATCCTCCCAGGATCCTGGAGAG
ACGTACCAAAGAGATCACAGTTCATTCCGGAAGCACTGTGGAACTGAAGTGCAGAGCAGAAGGTAGGC
CAAGCCCTACAGTTACCTGGATTCTTGCAAACCAAACAGTTGTCTCAGAATCATCCCAGGGAAGTAGG
CAGGCTGTGGTGACGGTTGACGGAACATTGGTCCTCCACAATCTCAGTATTTATGACCGTGGCTTTTA
CAAATGTGTGGCCAGCAACCCAGGTGGCCAGGATTCACTGCTGGTTAAAATACAAGTCATTGCAGCAC
CACCTGTTATTCTAGAGCAAAGGAGGCAAGTCATTGTAGGCACTTGGGGTGAAAGTTTAAAACTGCCC
TGTACTGCAAAAGGAACTCCTCAGCCCAGCGTTTACTGGGTCCTCTCTGATGGCACTGAAGTGAAACC
ATTACAGTTTACCAATTCCAAGTTGTTCTTATTTTCAAATGGGACTTTGTATATAAGAAACCTAGCCT
CTTCAGACAGGGGCACTTATGAATGCATTGCTACCAGTTCCACTGGTTCGGAGCGAAGAGTAGTAATG
CTTACAATGGAAGAGCGAGTGACCAGCCCCAGGATAGAAGCTGCATCCCAGAAAAGGACTGAAGTGAA
TTTTGGGGACAAATTACTACTGAACTGCTCAGCCACTGGGGAGCCCAAACCCCAAATAATGTGGAGGT
TACCATCCAAGGCTGTGGTCGACCAGCAGCATAGGGTGGGCAGCTGGATCCACGTCTACCCTAATGGA
TCCCTGTTTATTGGATCAGTAACAGAAAAAGACAGTGGTGTCTACTTGTGTGTGGCAAGAAACAAAAT
GGGGGATGATCTGATACTGATGCATGTTAGCCTAAGACTGAAACCTGCCAAAATTGACCACAAGCAGT
ATTTTAGAAAGCAAGTGCTCCATGGGAAAGATTTCCAAGTAGATTGCAAAGCTTCCGGCTCCCCAGTG
CCAGAGATATCTTGGAGTTTGCCTGATGGAACCATGATCAACAATGCAATGCAAGCCGATGACAGTGG
CCACAGGACTAGGAGATATACCCTTTTCAACAATGGAACTTTATACTTCAACAAAGTTGGGGTAGCGG
AGGAAGGAGATTATACTTGCTATGCCCAGAACAACCCTAAAGGGAAAGATGATGAGGTCCACTTAACA
GTTATAACAGCTGCTCCCCGGATAAGGCAGAGTAACAAAACCAACAAGAGAATCAAAGCTGGAGACAC
AGCTGTCCTTGACTGTGAGGTCACTGGGGATCCCAAAACCAAAATATTTTCATTGCTGCCTTCCAATG
ACATGATTTCCTTCTCCATTGATAGGTACACATTTCATGCCAATGGGTCTTTGACCATCAACAAAGTG
AAACTGCTCGATTCTGGAGAGTACGTATGTGTAGCCCGAAATCCCAGTGGGGATGACACCAAAATGTA
CAACTGGATGTGGTCTCTAAACCTCCATTAATCAAATGGTCTGTATACAAACAGAACTGTTATTAAAG
CCACAGCTGTGAGACATTCCAAAAAACACTTTGACTGCAGAGCTGAAGGGACACCATCTCCTGAAGTC
ATGTGGATCATGCCAGACAATATTTTCCTCACAGCCCCATACTATGGAAGCAGAATCACAGTCCATAA
AAATGGAACCTTGGAAATTAGGAATGTGAGGCTTTCAGATTCAGCCGACTTTATCTGTGTGGCCCGAA
ATGAAGGTGGAGAGAGCGTGTTGGTAGTACAGTTAGAAGTACTGGAAATGCTGAGAAGACCGACATTT
AGAAATCCATTTAATGAAAAAATAGTTGCCCAGCTGGGAAAGTCCACAGCATTGAATTGCTCTGTTGA
TGGTAACCCACCACCTGAAATAATCTGGATTTTACCAAATGGCACACGATTTTCCAATGGACCACAAA
GTTATCAGTATCTGAATAGCAAGCAATGGTTCTTTTATCATTTCTAAACAACTCGGGAGGATGCAGGA
AAATATCGCTGTGCAGCTAGGAATAAAGTTGGCTATATTGAGAAATTAGTCATATTAGAAATTGGCCA
GAAGCCAGTTATTCTTACCTATGCACCAGGGACAGTAAAAGGCATCAGTGGAGAATCTCTATCACTGC
ATTGTGTGTCTGATGGAATCCCTAAGCCAAATATCAAATGGACTATGCCAAGTGGTTATGTAGTAGAC
AGGCCTCAAATTAATGGGAAATACATATTGCATGACAATGGCACCTTAGTCATTAAAGAAGCAAGAGC
GCAGCCTTTCAGCTCCACTGTGTGGCCTTGGGAGTTCCCAAGCCAGAAATCACATGGGAGATGCCTGA
CCACTCCCTTCTCTCAACGGCAAGTAAAGAGAGGACACATGGAGTGAAGCAGCTTCACTTACAAGGTA
CCCTAGTCATTCAGAATCCCCALACCTCCGATTCTGGGATATACAAATGCACAGCAAAGAACCCACTT
GGTAGTGATTATGCAGCAACGTATATTCAAGTAAAATCTGACATGAATAATAAAGTC

| | | | |
|---|---|---|---|
| NOV29am, SNP13376525 of | SEQ ID NO:742 | 2617 aa | MW at 290219.2 kD |
| CG55776-01, Protein Sequence | SNP Pos: 163 | | SNP Change: His to Arg |

MKVKGRGITCLLVSFAVICLVATPGGKACPRRCACYMPTEVHCTFRYLTSIPDSIPPNVERINLGYNS
LVRLMETDFSGLTKLELLMLHSNGIHTPDKTFSDLQALQVRLMVLKMSYIAKVRKLQKDTFYGLRSLT
RLHMDHNNIEFINPEVFYGLNFLRLVRLEGNQLTKLHPDTFVSLSYLQIFKISFIKFLYLSDNFLTSL
PQEMVSYMPDLDSLYLHGNPWTCDCHLKWLSDWIQEKPGIYIVLPDVIKCKKDRSPSSAQQCPLCMNP
RTSKGKPLAMVSAAAFQCAKPTIDSSLKSKSLTILEDSSSAFISPQGFMAPFGSLTLNMTDQSGNEAN
PQLYYKYKQVAPKPEDIFTNIEADLRADPSWLMQDQISLQLNRTATTFSTLQIQYSSDAQITLPRAEM
RPVKHKWTMISRDNNTKLEHTVLVGGTVGLNCPGQGDPTPHVDWLLADGSKVPAPYVSEDGRILIDKS
GKLELQMADSFDTGVYHCISSNYDDADILTYRITVVEPLVEAYQENGIHHTVFIGETLDLPCHSTGIP
DASISWVIPGNNVLYQSSRDKKVLNNGTLRILQVTPKDQGYYRCVAANPSGVDFLIFQVSVKMKGQRP
LEHDGETEGSGLDESNPIAHLKEPPGAQLRTSALMEAEVGKHTSSTSKRHNYRELTLQRRGDSTHRRF
RENRRHFPPSARRIDPQHWAALLEKAKKNAMPDKRENTTVSPPPVVTQLPNIPGEEDDSSGMLALHEE
FMVPATKALNLPARTVTADSRTISDSPMTNINYGTEFSPVVNSQILPPEEPTDFKLSTAIKTTAMSKN
INPTMSSQIQGTTNQHSSTVFPLLLGATEFQDSDQMGRGREHFQSRPPITVRTMIKDVNVKNLSSTTN
KLLLESVNTTNSHQTSVREVSEPRHNHFYSHTTQILSTSTFPSDPHTAAHSQFPIPRNSTVNIPLFRR
FGRQRKIGGRGRIISPYRTPVLRRHRYSIFRSTTRGSSEKSTTAFSATVLNVTCLSCLPRERLTTATA
ALSFPSAAPITFPKADIARVPSEESTTLVQNPLLLLENKPSVEKTTPTIKYFRTEISQVTPTGAVMTY
APTSIPMEKTHKVNASYPRVSSTNEAKRDSVITSSLSGAITKPPMTIIAITRFSRRKIPWQQNFVNNH
ISKDSSTKSIISTQTAIPATTPTFPASVITYETQTERSRAQTIQREQEPQKKNRTDPNISPDQSSGFT
TPTAMTPPVLTTAETSVKPSVSAFTHSPPENTTGISSTISFHSRTLNLTDVIEELAQASTQTLKSTIA
SETTLSSKSHQSTTTRKAIIRHSTIPPFLSSSATLMPVPISPPFTQEAVTDNVATPISGLMTNTVVKL
HESSRHNAKPQQLVAEVATSPKVHPNAKFTIGTTHFIYSNLLHSTPMPALTTVKSQNSKLTPSPWAEN
QFWHKPYSEIAEKGKKPEVSMLATTGLSEATTLVSDWDGQKNTKKSDFDKKPVQEATTSKLLPFDSLS
RYIFEKPRIVGGKAASFTIPANSDAFLPCEAVGNPLPTIHWTRVPSGMSGLDLSKRKQNSRVQVLPNG
TLSQRVEIQDRGQYLCSASNLFGTDHLHVTLSKTVSYPPRILERRTKEITVHSGSTVELKCRAEGRPS
PTVTWILANQTVVSESSQGSRQAVVTVDGTLVLHNLSIYDRGFYKCVASNPGGQDSLLVKIQVIAAPP
VILEQRRQVIVGTWGESLKLPCTAKGTPQPSVYWVLSDGTEVKPLQFTNSKLFLFSNGTLYIRNLASS
DRGTYECIATSSTGSERRVVMLTMEERVTSPRIEAASQKRTEVNFGDKLLLNCSATGEPKPQIMWRLP
SKAVVDQQHRVGSWIHVYPNGSLFIGSVTEKDSGVYLCVARNKMGDDLILMHVSLRLKPAKIDHKQYF
RKQVLHGKDFQVDCKASGSPVPEISWSLPDGTMINNAMQADDSGHRTRRYTLFNNGTLYFNKVGVAEE
GDYTCYAQNTLGKDEMKVHLTVITAAPRIRQSNKTNKRIKAGDTAVLDCEVTGDPKPKIFWLLPSNDM
ISFSIDRYTFHANGSLTINKVKLLDSGEYVCVARNPSGDDTKMYKLDVVSKPPLINGLYTNRTVIKAT
AVRHSKKHFDCRAEGTPSPEVMWIMPDNIFLTAPYYGSRITVHKNGTLEIRNVRLSDSADFICVARNE
GGESVLVVQLEVLEMLRRPTFRNPFNEKIVAQLGKSTALNCSVDGNPPPEIIWILPNGTRFSNGPQSY
QYLIASNGSFIISKTTREDAGKYRCAARNKVGYIEKLVILEIGQKPVILTYAPGTVKGISGESLSLHC
VSDGIPKPNIKWTMPSGYVVDRPQINGKYILHDNGTLVIKEATAYDRGNYICKAQNSVGHTLITVPVM
IVAYPPRITNRPPRSIVTRTGAAFQLHCVALGVPKPEITWEMPDHSLLSTASKERTHGSEQLHLQGTL
VIQNPQTSDSGIYKCTAKNPLGSDYAATYIQVI

| | | |
|---|---|---|
| NOV29an, SNP13376526 | SEQ ID NO:743 | 7876 bP |
| of CG55776-01, | ORF Start: ATG at 6 | ORF Stop: TGA at 7857 |
| DNA Sequence | SNP Pos: 547 | SNP Change: G to A |

TCAGGATGAAGGTAAAAGGCAGAGGAATCACCTGCTTGCTGGTCTCCTTTGCTGTGATCTGCCTGGTC
GCCACCCCTGGGGGCAAGGCCTGTCCTCGCCGCTGTGCCTGTTATATGCCTACGGAGGTACACTGCAC
ATTTCGGTACCTGACTTCCATCCCAGACAGCATCCCGCCCAATGTGGAACGCATCAATTTAGGGTACA
ACAGCTTGGTTAGATTGATGGAAACAGATTTTTCTGGCCTGACCAAACTGGAGTTACTCATGCTTCAC
AGCAATGGCATTCACACAATCCCTGACAAGACCTTCTCAGATTTGCAGGCCTTGCAGGTGAGACTGAT
GGTCTTAAAAATGAGCTATAATAAAGTCCGAAAACTTCAGAAAGATACTTTTTATGGCCTCAGGAGCT
TGACACGATTGCACATGGACCACAACAATATTGAGTTTATAAACCCAGAGGTTTTTTATGGGCTCAAC
TTTCTCCGCCTGGTGCACTTGGAAGGAAATCAGCTCACTAAGCTCCACCCAGATACATTTGTCTCTTT
GAACTACCTCCAGATATTTAAAATCTCTTTCATTAAGTTCCTATACTTGTCTGATAACTTCCTGACCT
CCCTCCCTCAAGAGATGGTCTCCTATATGCCTGACCTAGACAGCCTTTACCTGCATGGAAACCCATGG
ACCTGTGATTGCCATTTAAAGTGGTTGTCTGACTGGATACAGGAGAAGCCAGGTATCTATATTGTNTT
ACCCTAGGACTTCTAAAGGCAAGCCGTTAGCTATGGTCTCAGCTGCAGCTTTCCAGTGTGCCAAGCCA
ACCATTGACTCATCCCTGAAATCAAAGAGCCTGACTATTCTGGAAGACAGTAGTTCTGCTTTCATCTC
TCCCCAAGGTTTCATGGCACCCTTTGGCTCCCTCACTTTGAATATGACAGATCAGTCTGGAAATGAAG
CTAACATGGTCTGCAGTATTCAAAAGCCCTCAAGGACATCACCCATTGCATTCACTGAAGAAAATGAC
TACATCGTGCTAAATACTTCATTTTCAACATTTTTGGTGTGCAACATAGATTACGGTCACATTCAGCC
AGTGTGGCAAATTTTGGCTTTGTACAGTGATTCTCCTCTGATACTAGAAAGGAGCCACTTGCTTAGTG
AAACACCGCAGCTCTATTACAAATATAAACAGGTGGCTCCTAAGCCTGAAGACATTTTTACCAACATA
GAGGCAGATCTCAGAGCAGATCCCTCTTGGTTAATGCAAGACCAAATTTCCTTGCAGCTGAACAGAAC
TGCCACCACATTCAGTACATTACAGATCCAGTACTCCAGTGATGCTCAAATCACTTTACCAAGAGCAG
AGATGAGGCCAGTGAAACACAAATGGACTATGATTTCAAGGGATAACAATACTAAGCTGGAACATACT
GTCTTGGTAGGTGGAACCGTTGGCCTGAACTGCCCAGGCCAAGGAGACCCCACCCCACACGTGGATTG
GCTTCTAGCTGATGGAAGTAAAGTGAGAGCCCCTTATGTCAGTGAGGATGGACGGATCCTAATAGACA
AAAGTGGAAAATTGGAACTCCAGATGGCTGATAGTTTTGACACAGGCGTATATCACTGTATAAGCAGC
AATTATGATGATGCAGATATTCTCACCTATAGGATAACTGTGGTAGAACCTTTGGTCGAAGCCTATCA
GGAAAATGGGATTCATCACACAGTTTTCATTGGTGAAACACTTGATCTTCCATGCCATTCTACTGGTA
AAAGTTCTAAACAATGGCACATTAAGAATATTACAGGTCACCCCGAAAGACCAAGGTTATTATCGCTG
TGTGGCAGCCAACCCATCAGGGGTTGATTTTTTGATTTTCCAAGTTTCAGTCAAGATGAAAGGACAAA
GGCCCTTGGAGCATGATGGAGAAACAGAGGGATCTGGACTTGATGAGTCCAATCCTATTGCTCATCTT
AAGGAGCCACCAGGTGCACAACTCCGTACATCTGCTCTGATGGAGGCTGAGGTTGGAAAACACACCTC
AAGCACAAGTAAGAGGCACAACTATCGGGAATTAACACTCCAGCGACGTGGAGATTCAACACATCGAC
GTTTTAGGGAGAATAGGAGGCATTTCCCTCCCTCTGCTAGGAGAATTGACCCACAACATTGGGCGGCA
CTGTTGGAGAAAGCTAAAAAGAATGCTATGCCAGACAAGCGAGAAAATACCACAGTGAGCCCACCCCC
AGTGGTCACCCAACTCCCAAACATACCTGGTGAAGAAGACGATTCCTCAGGCATGCTCGCTCTACATG
AGGAATTTATGGTCCCGGCCACTAAAGCTTTGAACCTTCCAGCAAGGACAGTGACTGCTGACTCCAGA
ACAATATCTGATAGTCCTATGACAAACATAAATTATGGCACAGAATTCTCTCCTGTTGTGAATTCACA
AATACTACCACCTGAAGAACCCACAGATTTCAAACTGTCTACTGCTATTAAAACTACAGCCATGTCAA
AGAATATAAACCCAACCATGTCAAGCCAAATACAAGGCACAACCAATCAACATTCATCCACTGTCTTT
CCACTGCTACTTGGAGCAACTGAATTTCAGGACTCTGACCAGATGGGAAGAGGAAGAGAGCATTTCCA
AAGTAGACCCCCAATAACAGTAAGGACTATGATCAAAGATGTCAATGTCAAAATGCTTAGTAGCACCA
CCAACAAACTATTATTAGAGTCAGTAAATACCACAAATAGTCATCAGACATCTGTAAGAGAAGTGAGT
GAACCCAGGCACAATCACTTCTATTCTCACACTACTCAAATACTTAGCACCTCCACGTTCCCTTCAGA
TCCACACACAGCTGCTCATTCTCAGTTTCCGATCCCTAGAAATAGTACAGTTAACATCCCGCTGTTCA
GACGCTTTGGGAGGCAGAGGAAAATTGGCGGAAGGGGGCGGATTATCAGCCCATATAGAACTCCAGTT
CTGCGACGGCATAGATACAGCATTTTCAGGTCAACAACCAGAGGTTCTTCTGAAAAAAGCACTACTGC
ATTCTCAGCCACAGTGCTCAATGTGACATGTCTGTCCTGTCTTCCCAGGGAGAGGCTCACCACTGCCA
CAGCAGCATTGTCTTTTCCAAGTGCTGCTCCCATCACCTTCCCCAAAGCTGACATTGCTAGAGTCCCA
TCAGAAGAGTCTACAACTCTAGTCCAGAATCCACTATTACTACTTGAGAACAAACCCAGTGTAGAGAA
AACAACACCCACAATAAAATATTTCAGGACTGAAATTTCCCAAGTGACTCCAACTGGTGCAGTCATGA
CATATGCTCCAACATCCATACCCATGGAAAAAACTCACAAAGTAAACGCCAGTTACCCACGTGTGTCT
AGCACCAATGAAGCTAAAAGAGATTCAGTGATTACATCGTCACTTTCAGGTGCTATCACCAAGCCACC
AATGACTATTATAGCCATTACAAGGTTTTCAAGAAGGAAAATTCCCTGGCAACAGAACTTTGTAAATA
ACCATAACCCAAAAGGCAGATTAAGGAATCAACATAAAGTTAGTTTACAAAAAAGCACAGCTGTGATG
CTTCCTAAAACATCTCCTGCTTTACCACAGAGACAAAGTCTACCCTCGCACCACACTACGACCAAAAC
ACACAATCCTGGAAGTCTTCCAACAAAGAAGGAGCTTCCCTTCCCACCCCTTAACCCTATGCTTCCTA
GTATTATAAGCAAAGACTCAAGTACAAAAAGCATCATATCAACGCAAACAGCAATACCAGCAACAACT
CCTACCTTCCCTGCATCTGTCATCACTTATGAAACCCAAACAGAGAGATCTAGAGCACAAACAATACA
AAGAGAACAGGAGCCTCAAAAGAAGAACAGGACTGACCCAAACATCTCTCCAGACCAGAGTTCTGGCT
TCACTACACCCACTGCTATGACACCTCCTGTTCTAACCACAGCCGAAACTTCAGTCAAGCCCAGTGTC
TCTGCATTCACTCATTCCCCACCAGAAAACACAACTGGGATTTCAAGCACAATCAGTTTTCATTCAAG
AACTCTTAATCTGACAGATGTGATTGAAGAACTAGCCCAAGCAAGTACTCAGACTTTGAAGAGCACAA
TTGCTTCTGAAACAACTTTGTCCAGCAAATCACACCAGAGTACCACAACTAGGAAAGCAATCATTAGA
CACTCAACCATACCACCATTCTTGAGCAGCAGTGCTACTCTAATGCCAGTTCCCATCTCCCCTCCCTT
TACTCAGAGAGCAGTTACTGACAACGTGGCGACTCCCATTTCCGGGCTTATGACAAATACAGTGGTCA
AGCTGCACGAATCCTCAAGGCACAATGCTAAACCACAGCAATTAGTAGCAGAGGTTGCAACATCCCCC
AAGGTTCACCCAAATGCCAAGTTCACAATTGGAACCACTCACTTCATCTACTCTAATCTGTTACATTC
TACTCCCATGCCAGCACTAACAACAGTTAAATCACAGAATTCTAAATTAACTCCATCTCCCTGGGCAG
AAAACCAATTTTGGCACAAACCATACTCAGAAATTGCTGAAAAAGGCAAAAAGCCAGAAGTAAGCATG
TTGGCTACTACAGGCCTGTCCGAGGCCACCACTCTTGTTTCAGATTGGGATGGACAGAAGAACACAAA
GAAGAGTGACTTTGATAAGAAACCAGTTCAAGAAGCAACAACTTCCAAACTCCTTCCCTTTGACTCTT
TGTCTAGGTATATATTTGAAAAGCCCAGGATAGTTGGAGGAAAAGCTGCAAGTTTTACTATTCCAGCT
AACTCAGATGCCTTTCTTCCCTGTGAAGCTGTTGGAAATCCCCTGCCCACCATTCATTGGACCAGAGT
CCCATCAGGTATGTCAGGACTTGATTTATCTAAGAGGAAACAGAATAGCAGGGTCCAGGTTCTCCCCA
ATGGTACCCTGTCCATCCAGAGGGTGGAAATTCAGGACCGCGGACAGTACTTGTGTTCCGCATCCAAT
CTGTTTGGCACAGACCACCTTCATGTCACCTTGTCTGTGGTTTCCTATCCTCCCAGGATCCTGGAGAG
ACGTACCAAAGAGATCACAGTTCATTCCGGAAGCACTGTGGAACTGAAGTGCAGAGCAGAAGGTAGGC
CAGGCTGTGGTGACGGTTGACGGAACATTGGTCCTCCACAATCTCAGTATTTATGACCGTGGCTTTTA
CAAATGTGTGGCCAGCAACCCAGGTGGCCAGGATTCACTGCTGGTTAAAATACAAGTCATTGCAGCAC
CACCTGTTATTCTAGAGCAAAGGAGGCAAGTCATTGTAGGCACTTGGGGTGAAAGTTTAAAACTGCCC
TGTACTGCAAAAGGAACTCCTCAGCCCAGCGTTTACTGGGTCCTCTCTGATGGCACTGAAGTGAAACC
ATTACAGTTTACCAATTCCAAGTTGTTCTTATTTTCAAATGGGACTTTGTATATAAGAAACCTAGCCT
CTTCAGACAGGGGCACTTATGAATGCATTGCTACCAGTTCCACTGGTTCGGAGCGAAGAGTAGTAATG
CTTACAATGGAAGAGCGAGTGACCAGCCCCAGGATAGAAGCTGCATCCCAGAAAAGGACTGAAGTGAA
TTTTGGGGACAAATTACTACTGAACTGCTCAGCCACTGGGGAGCCCAAACCCCAAATAATGTGGAGGT
TACCATCCAAGGCTGTGGTCGACCAGCAGCATAGGGTGGGCAGCTGGATCCACGTCTACCCTAATGGA
TCCCTGTTTATTGGATCAGTAACAGAAAAAGACAGTGGTGTCTACTTGTGTGTGGCAAGAAACAAAAT
GGGGGATGATCTGATACTGATGCATGTTAGCCTAAGACTGAAACCTGCCAAAATTGACCACAAGCAGT
ATTTTAGAAAGCAAGTGCTCCATGGGAAAGATTTCCAAGTAGATTGCAAAGCTTCCGGCTCCCCAGTG
CCAGAGATATCTTGGAGTTTGCCTGATGGAACCATGATCAACAATGCAATGCAAGCCGATGACAGTGG
CCACAGGACTAGGAGATATACCCTTTTCAACAATGGAACTTTATACTTCAACAAAGTTGGGGTAGCGG
AGGAAGGAGATTATACTTGCTATGCCCAGAACACCCTAGGGAAAGATGAAATGAAGGTCCACTTAACA
GTTATAACAGCTGCTCCCCGGATAAGGCAGAGTAACAAAACCAACAAGAGAATCAAAGCTGGAGACAC
AGCTGTCCTTGACTGTGAGGTCACTGGGGATCCCAAACCAAAAATATTTTGGTTGCTGCCTTCCAATG
ACATGATTTCCTTCTCCATTGATAGGTACACATTTCATGCCAATGGGTCTTTGACCATCAACAAAGTG
AAACTGCTCGATTCTGGAGAGTACGTATGTGTAGCCCGAAATCCCAGTGGGGATGACACCAAAATGTA
CAAACTGGATGTGGTCTCTAAACCTCCATTAATCAATGGTCTGTATACAAACAGAACTGTTATTAAAG
CCACAGCTGTGAGACATTCCAAAAAACACTTTGACTGCAGAGCTGAAGGGACACCATCTCCTGAAGTC
ATGTGGATCATGCCAGACAATATTTTCCTCACAGCCCCATACTATGGAAGCAGAATCACAGTCCATAA
AAATGGAACCTTGGAAATTAGGAATGTGAGGCTTTCAGATTCAGCCGACTTTATCTGTGTGGCCCGAA
ATGAAGGTGGAGAGAGCGTGTTGGTAGTACAGTTAGAAGTACTGGAAATGCTGAGAAGACCGACATTT
AGAAATCCATTTAATGAAAAAATAGTTGCCCAGCTGGGAAAGTCCACAGCATTGAATTGCTCTGTTGA
TGGTAACCCACCACCTGAAATAATCTGGATTTTACCAAATGGCACACGATTTTCCAATGGACCACAAA
GTTATCAGTATCTGATAGCAAGCAATGGTTCTTTTATCATTTCTAAAACAACTCGGGAGGATGCAGGA
AAATATCGCTGTGCAGCTAGGAATAAAGTTGGCTATATTGAGAAATTAGTCATATTAGAAATTGGCCA
GAAGCCAGTTATTCTTACCTATGCACCAGGGACAGTAAAAGGCATCAGTGGAGAATCTCTATCACTGC
ATTGTGTGTCTGATGGAATCCCTAAGCCAAATATCAAATGGACTATGCCAAGTGGTTATGTAGTAGAC
AGGCCTCAAATTAATGGGAAATACATATTGCATGACAATGGCACCTTAGTCATTAAAGAAGCAACAGC
TTATGACAGAGGAAACTATATCTGTAAGGCTCAAAATAGTGTTGGTCATACACTGATTACTGTTCCAG
TAATGATTGTAGCCTACCCTCCCCGAATTACAAATCGTCCACCCAGGAGTATTGTCACCAGGACAGGG
GCAGCCTTTCAGCTCCACTGTGTGGCCTTGGGAGTTCCCAAGCCAGAAATCACATGGGAGATGCCTGA
CCACTCCCTTCTCTCAACGGCAAGTAAAGAGAGGACACATGGAAGTGAGCAGCTTCACTTACAAGGTA
CCCTAGTCATTCAGAATCCCCAAACCTCCGATTCTGGGATATACAAATGCACAGCAAAGAACCCACTT
GGTAGTGATTATGCAGCAACGTATATTCAAGTAATCTGACATGAAATAATAAAGTC

| | | | |
|---|---|---|---|
| NOV29an, SNP13376526 of | SEQ ID NO:744 | 2617 aa | MW at 290227.2 kD |
| CG55776-01, | SNP Pos: 181 | | SNP Change: |
| Protein Sequence | | | Ser to Asn |

MKVKGRGITCLLVSFAVICLVATPGGKACPRRCACYMPTEVHCTFRYLTSTPDSIPPNVERINLGYNS
LVRLMETDFSGLTKLELLMLHSNGIHTIPDKTFSDLQALQVRLMVLKMSYNKVRKLQKDTFYGLRSLT
RLHMDHNNIEFINPEVFYGLNFLRLVHLEGNQLTKLHPDTFVSLNYLQIFKISFIKFLYLSDNFLTSL
PQEMVSYMPDLDSLYLHGNPWTCDCHLKWLSDWIQEKPGIYIVLPDVIKCKKDRSPSSAQQCPLCMNP
RTSKGKPLAMVSAAAFQCAKPTIDSSLKSKSLTILEDSSSAFISPQGFMAPFGSLTLNMTDQSGNEAN
MVCSIQKPSRTSPIAFTEENDYIVLNTSFSTFLVCNIDYGHIQPVWQILALYSDSPLILERSHLLSET
PQLYYKYKQVAPKPEDIFTNIEADLRADPSWLMQDQISLQLNRTATTFSTLQIQYSSDAQITLPRAEM
RPVKHKWTMISRDNNTKLEHTVLVGGTVGLNCPGQGDPTPHVDWLLADGSKVRAPYVSEDGRILIDKS
GKLELQMADSFDTGVYHCISSNYDDADILTYRITVVEPLVEAYQENGIHHTVFIGETLDLPCHSTGIP
DASISWVIPGNNVLYQSSRDKKVLNNGTLRILQVTPKDQGYYRCVAANPSGVDFLIFQVSVKMKGQRP
LEHDGETEGSGLDESNPIAHLKEPPGAQLRTSALMEAEVGKHTSSTSKRHNYRELTLQRRGDSTHRRF
RENRRHFPPSARRIDPQHWAALLEKAKKNAMPDKRENTTVSPPPVVTQLPNIPGEEDDSSGMLALHEE
FMVPATKALNLPARTVTADSRTISDSPMTNINYGTEFSPVVNSQILPPEEPTDFKLSTAIKTTAMSKN
INPTMSSQIQGTTNQHSSTVFPLLLGATEFQDSDQMGRGREHFQSRPPITVRTMIKDVNVKMLSSTTN
KLLLESVNTTNSHQTSVREVSEPRHNHFYSHTTQILSTSTFPSDPHTAAHSQFPIPRNSTVNIPLFRR
FGRQRKIGGRGRIISPYRTPVLRRHRYSIFRSTTRGSSEKSTTAFSATVLNVTCLSCLPRERLTTATA
ALSFPSAAPITFPKADIARVPSEESTTLVQNPLLLLENKPSVEKTTPTIKYFRTEISQVTPTGAVMTY
APTSIPMEKTHKVNASYPRVSSTNEAKRDSVITSSLSGAITKPPMTIIAITRFSRRKIPWQQNFVNNH
NPKGRLRNQHKVSLQKSTAVMLPKTSPALPQRQSLPSHHTTTKTHNPGSLPTKKELPFPPLNPMLPSI
ISKDSSTKSIISTQTAIPATTPTFPASVITYETQTERSRAQTIQREQEPQKKNRTDPNISPDQSSGFT
TPTAMTPPVLTTAETSVKPSVSAFTHSPPENTTGISSTISFHSRTLNLTDVIEELAQASTQTLKSTIA
SETTLSSKSHQSTTTRKAIIRHSTIPPFLSSSATLMPVPISPPFTQRAVTDNVATPISGLMTNTVVKL
HESSRHNAKPQQLVAEVATSPKVHPNAKFTIGTTHFIYSNLLHSTPMPALTTVKSQNSKLTPSPWAEN
QFWHKPYSEIAEKGKKPEVSMLATTGLSEATTLVSDWDGQKNTKKSDFDKKPVQEATTSKLLPFDSLS
RYIFEKPRIVGGKAASFTIPANSDAFLPCEAVGNPLPTIHWTRVPSGMSGLDLSKRKQNSRVQVLPNG
TLSIQRVEIQDRGQYLCSASNLFGTDHLHVTLSVVSYPPRILERRTKEITVHSGSTVELKCRAEGRPS
PTVTWILANQTVVSESSQGSRQAVVTVDGTLVLHNLSIYDRGFYKCVASNPGGQDSLLVKIQVIAAPP
VILEQRRQVIVGTWGESLKLPCTAKGTPQPSVYWVLSDGTEVKPLQFTNSKLFLFSNGTLYIRNLASS
DRGTYECIATSSTGSERRVVMLTMEERVTSPRIEAASQKRTEVNFGDKLLLNCSATGEPKPQIMWRLP
SKAVVDQQHRVGSWIHVYPNGSLFIGSVTEKDSGVYLCVAAAKMGDDLILMHVSLRLKPAKIDHKQYF
RKQVLHGKDFQVDCKASGSPVPEISWSLPDGTMINNAMQADDSGHRTRRYTLFNNGTLYFNKVGVAEE
GDYTCYAQNTLGKDEMKVHLTVITAAPRIRQSNKTNKRIKAGDTAVLDCEVTGDPKPKIFWLLPSNDM
ISFSIDRYTFHANGSLTINKVKLLDSGEYVCVARNPSGDDTKMYKLDVVSKPPLINGLYTNRTVIKAT
AVRHSKKHFDCRAEGTPSPEVMWIMPDNIFLTAPYYGSRITVHKNGTLEIRNVRLSDSADFICVARNE
GGESVLVVQLEVLEMLRRPTFRNPFNEKIVAQLGKSTALNCSVDGNPPPEIIWILPNGTRFSNGPQSY
QYLIASNGSFIISKTTREDAGKYRCAARNKVGYIEKLVILEIGQKPVILTYAPGTVKGISGESLSLHC
VSDGIPKPNIKWTMPSGYVVDRPQINGKYILHDNGTLVIKEATAYDRGNYICKAQNSVGHTLITVPVM
IVAYPPRITNRPPRSIVTRTGAAFQLHCVALGVPKPEITWEMPDHSLLSTASKERTHGSEQLHLQGTL
IVIQNPQTSDSGIYKCTAKNPLGSDYAATYIQVI

| | | |
|---|---|---|
| NOV29ao, SNP13377626 | SEQ ID NO:745 | 7876 bP |
| of CG55776-01, | ORF Start: ATG at 6 | ORF Stop: TGA at 7857 |
| DNA Sequence | SNP Pos: 4405 | SNP Change: C to T |

TCAGGATGAAGGTAAAAGGCAGAGGAATCACCTGCTTGCTGGTCTCCTTTGCTGTGATCTGCCTGGTC
GCCACCCCTGGGGGCAAGGCCTGTCCTCGCCGCTGTGCCTGTTATATGCCTACGGAGGTACACTGCAC
ATTTCGGTACCTGACTTCCATCCCAGACAGCATCCCGCCCAATGTGGAACGCATCAATTTAGGGTACA
ACAGCTTGGTTAGATTGATGGAAACAGATTTTTCTGGCCTGACCAAACTGGAGTTACTCATGCTTCAC
AGCAATGGCATTCACACAATCCCTGACAAGACCTTCTCAGATTTGCAGGCCTTGCAGGTGAGACTGAT
TGACACGATTGCACATGGACCACAACAATATTGAGTTTATAAACCCAGAGGTTTTTTATGGGCTCAAC
TTTCTCCGCCTGGTGCACTTGGAAGGAAATCAGCTCACTAAGCTCCACCCAGATACATTTGTCTCTTT
GAGCTACCTCCAGATATTTAAAATCTCTTTCATTAAGTTCCTATACTTGTCTGATAACTTCCTGACCT
CCCTCCCTCAAGAGATGGTCTCCTATATGCCTGACCTAGACAGCCTTTACCTGCATGGAAACCCATGG
ACCTGTGATTGCCATTTAAAGTGGTTGTCTGACTGGATACAGGAGAAGCCAGGTATCTATATTGTNTT
ACCAGATGTAAAAAAATGCAAAAAAGATAGAAGTCCCTCTAGTGCTCAGCAGTGTCCACTTTGCATGA
ACCCTAGGACTTCTAAAGGCAAGCCGTTAGCTATGGTCTCAGCTGCAGCTTTCCAGTGTGCCAAGCCA
ACCATTGACTCATCCCTGAAATCAAAGAGCCTGACTATTCTGGAAGACAGTACTTCTGCTTTCATCTC
TCCCCAAGGTTTCATGGCACCCTTTGGCTCCCTCACTTTGAATATGACAGATCAGTCTGGAAATGAAG
CTAACATGGTCTGCAGTATTCAAAAGCCCTCAAGGACATCACCCATTGCATTCACTGAAGAAAATGAC
TACATCGTGCTAAATACTTCATTTTCAACATTTTTGGTGTGCAACATAGATTACGGTCACATTCAGCC
AGTGTGGCAAATTTTGGCTTTGTACAGTGATTCTCCTCTGATACTAGAAAGGAGCCACTTGCTTAGTG
AAACACCGCAGCTCTATTACAAATATAAACAGGTGGCTCCTAAGCCTGAAGACATTTTTACCAACATA
GAGGCAGATCTCAGAGCAGATCCCTCTTGGTTAATGCAAGACCAAATTTCCTTGCAGCTGAACAGAAC
TGCCACCACATTCAGTACATTACAGATCCAGTACTCCAGTGATGCTCAAATCACTTTACCAAGAGCAG
AGATGAGGCCAGTGAAACACAAATGGACTATGATTTCAAGGGATAACAATACTAAGCTGGAACATACT
GTCTTGGTAGGTGGAACCGTTGGCCTGAACTGCCCAGGCCAAGGAGACCCCACCCCACACGTGGATTG
GCTTCTAGCTGATGGAAGTAAAGTGAGAGCCCCTTATGTCAGTGAGGATGGACGGATCCTAATAGACA
AAAGTGGAAAATTGGAACTCCAGATGGCTGATAGTTTTGACACAGGCGTATATCACTGTATAAGCAGC
AATTATGATGATGCAGATATTCTCACCTATAGGATAACTGTGGTAGAACCTTTGGTCGAAGCCTATCA
GGAAAATGGGATTCATCACACAGTTTTCATTGGTGAAACACTTGATCTTCCATGCCATTCTACTGGTA
TCCCAGATGCCTCTATTAGCTGGGTTATTCCAGGAAACAATGTGCTCTATCAGTCATCAAGAGACAAG
AAAGTTCTAAACAATGGCACATTAAGAATATTACAGGTCACCCCGAAAGACCAAGGTTATTATCGCTG
TGTGGCAGCCAACCCATCAGGGGTTGATTTTTTGATTTTCCAAGTTTCAGTCAAGATGAAAGGACAAA
GGCCCTTGGAGCATGATGGAGAAACAGAGGGATCTGGACTTGATGAGTCCAATCCTATTGCTCATCTT
AAGGAGCCACCAGGTGCACAACTCCGTACATCTGCTCTGATGGAGGCTGAGGTTGGAAAACACACCTC
AAGCACAAGTAAGAGGCACAACTATCGGGAATTAACACTCCAGCGACGTGGAGATTCAACACATCGAC
GTTTTAGGGAGAATAGGAGGCATTTCCCTCCCTCTGCTAGGAGAATTGACCCACAACATTGGGCGGCA
CTGTTGGAGAAAGCTAAAAAGAATGCTATGCCAGACAAGCGAGAAAATACCACAGTGAGCCCACCCCC
AGTGGTCACCCAACTCCCAAACATACCTGGTGAAGAAGACGATTCCTCAGGCATGCTCGCTCTACATG
AGGAATTTATGGTCCCGGCCACTAAAGCTTTGAACCTTCCAGCAAGGACAGTGACTGCTGACTCCAGA
ACAATATCTGATAGTCCTATGACAAACATAAATTATGGCACAGAATTCTCTCCTGTTGTGAATTCACA
AATACTACCACCTGAAGAACCCACAGATTTCAAACTGTCTACTGCTATTAAAACTACAGCCATGTCAA
AGAATATAAACCCAACCATGTCAAGCCAAATACAAGGCACAACCAATCAACATTCATCCACTGTCTTT
CCACTGCTACTTGGAGCAACTGAATTTCAGGACTCTGACCAGATGGGAAGAGGAAGAGAGCATTTCCA
AAGTAGACCCCCAATAACAGTAAGGACTATGATCAAAGATGTCAATGTCAAAATGCTTAGTAGCACCA
CCAACAAACTATTATTAGAGTCAGTAAATACCACAAATAGTCATCAGACATCTGTAAGAGAAGTGAGT
GAACCCAGGCACAATCACTTCTATTCTCACACTACTCAAATACTTAGCACCTCCACGTTCCCTTCAGA
TCCACACACAGCTGCTCATTCTCAGTTTCCGATCCCTAGAAATAGTACAGTTAACATCCCGCTGTTCA
GACGCTTTGGGAGGCAGAGGAAAATTGGCGGAAGGGGGCGGATTATCAGCCCATATAGAACTCCAGTT
CTGCGACGGCATAGATACAGCATTTTCAGGTCAACAACCAGAGGTTCTTCTGAAAAAAGCACTACTGC
ATTCTCAGCCACAGTGCTCAATGTGACATGTCTGTCCTGTCTTCCCAGGGAGAGGCTCACCACTGCCA
CAGCAGCATTGTCTTTTCCAAGTGCTGCTCCCATCACCTTCCCCAAAGCTGACATTGCTAGAGTCCCA
TCAGAAGAGTCTACAACTCTAGTCCAGAATCCACTATTACTACTTGAGAACAAACCCAGTGTAGAGAA
AACAACACCCACAATAAAATATTTCAGGACTGAAATTTCCCAAGTGACTCCAACTGGTGCAGTCATGA
CATATGCTCCAACATCCATACCCATGGAAAAAACTCACAAAGTAAACGCCAGTTACCCACGTGTGTCT
AATGACTATTATAGCCATTACAAGGTTTTCAAGAAGGAAAATTCCCTGGCAACAGAACTTTGTAAATA
ACCATAACCCAAAAGGCAGATTAAGGAATCAACATAAAGTTAGTTTACAAAAAAGCACAGCTGTGATG
CTTCCTAAAACATCTCCTGCTTTACCACAGAGACAAAGTCTCCCCTCGCACCACACTACGACCAAAAC
ACACAATCCTGGAAGTCTTCCAACAAAGAAGGAGCTTCCCTTCCCACCCCTTAACCCTATGCTTCCTA
GTATTATAAGCAAAGACTCAAGTACAAAAAGCATCATATCAACGCAAACAGCAATACCAGCAACAACT
CCTACCTTCCCTGCATCTGTCATCACTTATGAAACCCAAACAGAGAGATCTAGAGCACAAACAATACA
AAGAGAACAGGAGCCTCAAAAGAAGAACAGGACTGACCCAAACATCTCTCCAGACCAGAGTTCTGGCT
TCACTACACCCACTGCTATGACACCTCCTGTTCTAACCACAGCCGAAACTTCAGTCAAGCCCAGTGTC
TCTGCATTCACTCATTCCCCACCAGAAAACACAACTGGGATTTCAAGCACAATCAGTTTTCATTCAAG
AACTCTTAATCTGACAGATGTGATTGAAGAACTAGCCCAAGCAAGTACTCAGACTTTGAAGAGCACAA
TTGCTTCTGAAACAACTTTGTCCAGCAAATCACACCAGAGTACCACAACTAGGAAAGCAATCATTAGA
CACTCAACCATACCACCATTCTTGAGCAGCAGTGCTACTCTAATGCCAGTTCTCATCTCCCCTCCCTT
TACTCAGAGAGCAGTTACTGACAACGTGGCGACTCCCATTTCCGGGCTTATGACAAATACAGTGGTCA
AGCTGCACGAATCCTCAAGGCACAATGCTAAACCACAGCAATTAGTAGCAGAGGTTGCAACATCCCCC
AAGGTTCACCCAAATGCCAAGTTCACAATTGGAACCACTCACTTCATCTACTCTAATCTGTTACATTC
AAAACCAATTTTGGCACAAACCATACTCAGAAATTGCTGAAAAAGGCAAAAAGCCAGAAGTAAGCATG
TTGGCTACTACAGGCCTGTCCGAGGCCACCACTCTTGTTTCAGATTGGGATGGACAGAAGAACACAAA
GAAGAGTGACTTTGATAAGAAACCAGTTCAAGAAGCAACAACTTCCAAACTCCTTCCCTTTGACTCTT
CCCATCAGGTATGTCAGGACTTGATTTATCTAAGAGGAAACAGAATAGCAGGGTCCAGGTTCTCCCCA
ATGGTACCCTGTCCATCCAGAGGGTGGAAATTCAGGACCGCGGACAGTACTTGTGTTCCGCATCCAAT
CTGTTTGGCACAGACCACCTTCATGTCACCTTGTCTGTGGTTTCCTATCCTCCCAGGATCCTGGAGAG
ACGTACCAAAGAGATCACAGTTCATTCCGGAAGCACTGTGGAACTGAAGTGCAGAGCAGAAGGTAGGC
CAAGCCCTACAGTTACCTGGATTCTTGCAAACCAAACAGTTGTCTCAGAATCATCCCAGGGAAGTAGG
CAGGCTGTGGTGACGGTTGACGGAACATTGGTCCTCCACAATCTCAGTATTTATGACCGTGGCTTTTA
CAAATGTGTGGCCAGCAACCCAGGTGGCCAGGATTCACTGCTGGTTAAAATACAAGTCATTGCAGCAC
CACCTGTTATTCTAGAGCAAAGGAGGCAAGTCATTGTAGGCACTTGGGGTGAAAGTTTAAAACTGCCC
TGTACTGCAAAAGGAACTCCTCAGCCCAGCGTTTACTGGGTCCTCTCTGATGGCACTGAAGTGAAACC
ATTACAGTTTACCAATTCCAAGTTGTTCTTATTTTCAAATGGGACTTTGTATATAAGAAACCTAGCCT
CTTCAGACAGGGGCACTTATGAATGCATTGCTACCAGTTCCACTGGTTCGGAGCGAAGAGTAGTAATG
CTTACAATGGAAGAGCGAGTGACCAGCCCCAGGATAGAAGCTGCATCCCAGAAAAGGACTGAAGTGAA
TTTTGGGGACAAATTACTACTGAACTGCTCAGCCACTGGGGAGCCCAAACCCCAAATAATGTGGAGGT
TACCATCCAAGGCTGTGGTCGACCAGCAGCATAGGGTGGGCAGCTGGATCCACGTCTACCCTAATGGA
TCCCTGTTTATTGGATCAGTAACAGAAAAAGACAGTGGTGTCTACTTGTGTGTGGCAAGAAACAAAAT
GGGGGATGATCTGATACTGATGCATGTTAGCCTAAGACTGAAACCTGCCAAAATTGACCACAAGCAGT
ATTTTAGAAAGCAAGTGCTCCATGGGAAAGATTTCCAAGTAGATTGCAAAGCTTCCGGCTCCCCAGTG
CCAGAGATATCTTGGAGTTTGCCTGATGGAACCATGATCAACAATGCAATGCAAGCCGATGACAGTGG
CCACAGGACTAGGAGATATACCCTTTTCAACAATGGAACTTTATACTTCAACAAAGTTGGGGTAGCGG
AGGAAGGAGATTATACTTGCTATGCCCAGAACACCCTAGGGAAAGATGAAATGAAGGTCCACTTAACA
GTTATAACAGCTGCTCCCCGGATAAGGCAGAGTAACAAAACCAACAAGAGAATCAAAGCTGGAGACAC
AGCTGTCCTTGACTGTGAGGTCACTGGGGATCCCAAACCAAAAATATTTTGGTTGCTGCCTTCCAATG
ACATGATTTCCTTCTCCATTGATAGGTACACATTTCATGCCAATGGGTCTTTGACCATCAACAAAGTG
AAACTGCTCGATTCTGGAGAGTACGTATGTGTAGCCCGAAATCCCAGTGGGGATGACACCAAAATGTA
CAAACTGGATGTGGTCTCTAAACCTCCATTAATCAATGGTCTGTATACAAACAGAACTGTTATTAAAG
CCACAGCTGTGAGACATTCCAAAAAACACTTTGACTGCAGAGCTGAAGGGACACCATCTCCTGAAGTC
ATGTGGATCATGCCAGACAATATTTTCCTCACAGCCCCATACTATGGAAGCAGAATCACAGTCCATAA
AAATGGAACCTTGGAAATTAGGAATGTGAGGCTTTCAGATTCAGCCGACTTTATCTGTGTGGCCCGAA
ATGAAGGTGGAGAGAGCGTGTTGGTAGTACAGTTAGAAGTACTGGAAATGCTGAGAAGACCGACATTT
AGAAATCCATTTAATGAAAAAATAGTTGCCCAGCTGGGAAAGTCCACAGCATTGAATTGCTCTGTTGA
TGGTAACCCACCACCTGAAATAATCTGGATTTTACCAAATGGCACACGATTTTCCAATGGACCACAAA
GTTATCAGTATCTGATAGCAAGCAATGGTTCTTTTATCATTTCTAAAACAACTCGGGAGGATGCAGGA
AAATATCGCTGTGCAGCTAGGAATAAAGTTGGCTATATTGAGAAATTAGTCATATTAGAAATTGGCCA
GAAGCCAGTTATTCTTACCTATGCACCAGGGACAGTAAAAGGCATCAGTGGAGAATCTCTATCACTGC
ATTGTGTGTCTGATGGAATCCCTAAGCCAAATATCAAATGGACTATGCCAAGTGGTTATGTAGTAGAC
AGGCCTCAAATTAATGGGAAATACATATTGCATGACAATGGCACCTTAGTCATTAAAGAAGCAACAGC
TTATGACAGAGGAAACTATATCTGTAAGGCTCAAAATAGTGTTGGTCATACACTGATTACTGTTCCAG
TAATGATTGTAGCCTACCCTCCCCGAATTACAAATCGTCCACCCAGGAGTATTGTCACCAGGACAGGG
GCAGCCTTTCAGCTCCACTGTGTGGCCTTGGGAGTTCCCAAGCCAGAAATCACATGGGAGATGCCTGA
CCACTCCCTTCTCTCAACGGCAAGTAAAGAGAGGACACATGGAAGTGAGCAGCTTCACTTACAAGGTA
CCCTAGTCATTCAGAATCCCCAAACCTCCGATTCTGGGATATACAAATGCACAGCAAAGAACCCACTT
GGTAGTGATTATGCAGCAACGTATATTCAAGTAATCTQACATGAAATAATAAAGTC

| | | | |
|---|---|---|---|
| NOV29ao, SNP13377626 of | SEQ ID NO:746 | 2617 aa | MW at 290216.2 kD |
| CG55776-01, Protein Sequence | SNP Pos: 1467 | | SNP Change: Pro to Leu |

MKVKGRGITCLLVSFAVICLVATPGGKACPRRCACYMPTEVHCTFRYLTSIPDSIPPNVERINLGYNS
LVRLMETDFSGLTKLELLMLHSNGIHTIPDKTFSDLQALQVRLMVLKMSYNKVRKLQKDTFYGLRSLT
RLHMDHNNIEFINPEVFYGLNFLRLVHLEGNQLTKLHPDTFVSLSYLQIFKISFIKFLYLSDNFLTSL
RTSKGKPLAMVSAAAFQCAKPTIDSSLKSKSLTILEDSSSAFTSPQGFMAPFGSLTLNNTDQSGNEAN
MVCSIQKPSRTSPIAFTEENDYIVLNTSFSTFLVCNIDYGHIQPVWQILALYSDSPLILERSHLLSET
PQLYYKYKQVAPKPEDIFTNIEADLRADPSWLMQDQISLQLNRTATTFSTLQIQYSSDAQITLPRAEM
RPVKHKWTMISRDNNTKLEHTVLVGGTVGLNCPGQGDPTPHVDWLLADGSKVRAPYVSEDGRILIDKS
GKLELQMADSFDTGVYHCISSNYDDADILTYRITVVEPLVEAYQENGIHHTVFIGETLDLPCHSTGIP
DASISWVIPGNNVLYQSSRDKKVLNNGTLRILQVTPKDQGYYRCVAANPSGVDFLIFQVSVKMKGQRP
LEHDGETEGSGLDESNPIAHLKEPPGAQLRTSALMEAEVGKHTSSTSKRHNYRELTLQRRGDSTHRRF
RENRRHFPPSARRIDPQHWAALLEKAKKNAMPDKRENTTVSPPPVVTQLPNIPGEEDDSSGMLALHEE
FMVPATKALNLPARTVTADSRTISDSPMTNINYGTEFSPVVNSQILPPEEPTDFKLSTAIKTTAMSKN
KLLLESVNTTNSHQTSVREVSEPRHNHFYSHTTQILSTSTFPSDPHTAAHSQFPIPRNSTVNIPLFRR
FGRQRKIGGRGRIISPYRTPVLRRHRYSIFRSTTRGSSEKSTTAFSATVLNVTCLSCLPRERLTTATA
ALSFPSAAPITFPKADIARVPSEESTTLVQNPLLLLENKPSVEKTTPTIKYFRTEISQVTPTGAVMTY
APTSIPMEKTHKVNASYPRVSSTNEAKRDSVITSSLSGAITKPPMTIIAITRFSRRKIPWQQNFVNNH
NPKGRLRNQHKVSLQKSTAVMLPKTSPALPQRQSLPSHHTTTKTHNPGSLPTKKELPFPPLNPMLPSI
ISKDSSTKSIISTQTAIPATTPTFPASVITYETQTERSRAQTIQREQEPQKKNRTDPNISPDQSSGFT
TPTAMTPPVLTTAETSVKPSVSAFTHSPPENTTGISSTISFHSRTLNLTDVIEELAQASTQTLKSTIA
SETTLSSKSHQSTTTRKAIIRHSTIPPFLSSSATLMPVLISPPFTQRAVTDNVATPISGLMTNTVVKL
HESSRHNAKPQQLVAEVATSPKVHPNAKFTIGTTHFIYSNLLHSTPMPALTTVKSQNSKLTPSPWAEN
QFWHKPYSEIAEKGKKPEVSMLATTGLSEATTLVSDWDGQKNTKKSDFDKKPVQEATTSKLLPFDSLS
RYIFEKPRIVGGKAASFTIPANSDAFLPCEAVGNPLPTIHWTRVPSGMSGLDLSKRKQNSRVQVLPNG
TLSIQRVEIQDRGQYLCSASNLFGTDHLHVTLSVVSYPPRILERRTKEITVHSGSTVELKCRAEGRPS
PTVTWILANQTVVSESSQGSRQAVVTVDGTLVLHNLSIYDRGFYKCVASNPGGQDSLLVKIQVIAAPP
VILEQRRQVIVGTWGESLKLPCTAKGTPQPSVYWVLSDGTEVKPLQFTNSKLFLFSNGTLYIRNLASS
DRGTYECIATSSTGSERRVVMLTMEERVTSPRIEAASQKRTEVNFGDKLLLNCSATGEPKPQIMWRLP
SKAVVDQQHRVGSWIHVYPNGSLFIGSVTEKDSGVYLCVARNKMGDDLILMHVSLRLKPAKIDHKQYF
RKQVLHGKDFQVDCKASGSPVPEISWSLPDGTMINNAMQADDSGHRTRRYTLFNNGTLYFNKVGVAEE
GDYTCYAQNTLGKDEMKVHLTVITAAPRIRQSNKTNKRIKAGDTAVLDCEVTGDPKPKIFWLLPSNDM
ISFSIDRYTFHANGSLTINKVKLLDSGEYVCVARNPSGDDTKMYKLDVVSKPPLINGLYTNRTVIKAT
AVRHSKKHFDCRAEGTPSPEVMWIMPDNIFLTAPYYGSRITVHKNGTLEIRNVRLSDSADFICVARNE
GGESVLVVQLEVLEMLRRTFRNPFNIEKIVAQLGKSTALNCSVDGNPPPEIIWILPNGTRFSNGPQSY
QYLIASNGSFIISKTTREDAGKYRCAARNKVGYIEKLVILEIGQKPVILTYAPGTVKGISGESLSLHC
VSDGIPKPNIKWTMPSGYVVDRPQINGKYILHDNGTLVIKEATAYDRGNYICKAQNSVGHTLITVPVM
IVAYPPRITNRPPRSIVTRTGAAFQLHCVALGVPKPEITWEMPDHSLLSTASKERTHGSEQLHLQGTL
VIQNPQTSDSGIYKCTAKNPLGSDYAATYIQVI

| | | | |
|---|---|---|---|
| NOV29ap, SNP13377641 | SEQ ID NO:747 | 7876 bP | |
| of CG55776-01, | ORF Start: ATG at 6 | ORF Stop: TGA at 7857 | |
| DNA Sequence | | | SNP Pos: 6716 ISNP Change: C to T |

TCAGGATGAAGGTAAAAGGCAGAGGAATCACCTGCTTGCTGGTCTCCTTTGCTGTGATCTGCCTGGTC
GCCACCCCTGGGGGCAAGGCCTGTCCTCGCCGCTGTGCCTGTTATATGCCTACGGAGGTACACTGCAC
ATTTCGGTACCTGACTTCCATCCCAGACAGCATCCCGCCCAATGTGGAACGCATCAATTTAGGGTACA
ACAGCTTGGTTAGATTGATGGAAACAGATTTTTCTGGCCTGACCAAACTGGAGTTACTCATGCTTCAC
AGCAATGGCATTCACACAATCCCTGACAAGACCTTCTCAGATTTGCAGGCCTTGCAGGTGAGACTGAT
GGTCTTAAAAATGAGCTATAATAAAGTCCGAAAACTTCAGAAAGATACTTTTTATGGCCTCAGGAGCT
TGACACGATTGCACATGGACCACAACAATATTGAGTTTATAAACCCAGAGGTTTTTTATGGGCTCAAC
TTTCTCCGCCTGGTGCACTTGGAAGGAAATCAGCTCACTAAGCTCCACCCAGATACATTTGTCTCTTT
GAGCTACCTCCAGATATTTAAAATCTCTTTCATTAAGTTCCTATACTTGTCTGATAACTTCCTGACCT
CCCTCCCTCAAGAGATGGTCTCCTATATGCCTGACCTAGACAGCCTTTACCTGCATGGAAACCCATGG
ACCTGTGATTGCCATTTAAAGTGGTTGTCTGACTGGATACAGGAGAAGCCAGGTATCTATATTGTNTT
ACCAGATGTAATAAAATGCAAAAAAGATAGAAGTCCCTCTAGTGCTCAGCAGTGTCCACTTTGCATGA
ACCCTAGGACTTCTAAAGGCAAGCCGTTAGCTATGGTCTCAGCTGCAGCTTTCCAGTGTGCCAAGCCA
ACCATTGACTCATCCCTGAAATCAAAGAGCCTGACTATTCTGGAAGACAGTAGTTCTGCTTTCATCTC
TCCCCAAGGTTTCATGGCACCCTTTGGCTCCCTCACTTTGAATATGACAGATCAGTCTGGAAATGAAG
CTAACATGGTCTGCAGTATTCAAAAGCCCTCAAGGACATCACCCATTGCATTCACTGAAGAAAATGAC
TACATCGTGCTAAATACTTCATTTTCAACATTTTTGGTGTGCAACATAGATTACGGTCACATTCAGCC
AGTGTGGCAAATTTTGGCTTTGTACAGTGATTCTCCTCTGATACTAGAAAGGAGCCACTTGCTTAGTG
AAACACCGCAGCTCTATTACAAATATAAACAGGTGGCTCCTAAGCCTGAAGACATTTTTACCAACATA
GAGGCAGATCTCAGAGCAGATCCCTCTTGGTTAATGCAAGACCAAATTTCCTTGCAGCTGAACAGAAC
TGCCACCACATTCAGTACATTACAGATCCAGTACTCCAGTGATGCTCAAATCACTTTACCAAGAGCAG
AGATGAGGCCAGTGAAACACAAATGGACTATGATTTCAAGGGATAACAATACTAAGCTGGAACATACT
GTCTTGGTAGGTGGAACCGTTGGCCTGAACTGCCCAGGCCAAGGAGACCCCACCCCACACGTGGATTG
GCTTCTAGCTGATGGAAGTAAAGTGAGAGCCCCTTATGTCAGTGAGGATGGACGGATCCTAATAGACA
AAAGTGGAAAATTGGAACTCCAGATGGCTGATAGTTTTGACACAGGCGTATATCACTGTATAAGCAGC
AATTATGATGATGCAGATATTCTCACCTATAGGATAACTGTGGTAGAACCTTTGGTCGAAGCCTATCA
GGAAAATGGGATTCATCACACAGTTTTCATTGGTGAAACACTTGATCTTCCATGCCATTCTACTGGTA
TCCCAGATGCCTCTATTAGCTGGGTTATTCCAGGAAACAATGTGCTCTATCAGTCATCAAGAGACAAG
AAAGTTCTAAACAATGGCACATTAAGAATATTACAGGTCACCCCGAAAGACCAAGGTTATTATCGCTG
TGTGGCAGCCAACCCATCAGGGGTTGATTTTTTGATTTTCCAAGTTTCAGTCAAGATGAAAGGACAAA
GGCCCTTGGAGCATGATGGAGAAACAGAGGGATCTGGACTTGATGAGTCCAATCCTATTGCTCATCTT
AAGGAGCCACCAGGTGCACAACTCCGTACATCTGCTCTGATGGAGGCTGAGGTTGGAAAACACACCTC
AAGCACAAGTAAGAGGCACAACTATCGGGAATTAACACTCCAGCGACGTGGAGATTCAACACATCGAC
GTTTTAGGGAGAATAGGAGGCATTTCCCTCCCTCTGCTAGGAGAATTGACCCACAACATTGGGCGGCA
CTGTTGGAGAAAGCTAAAAAGAATGCTATGCCAGACAAGCGAGAAAATACCACAGTGAGCCCACCCCC
AGTGGTCACCCAACTCCCAAACATACCTGGTGAAGAAGACGATTCCTCAGGCATGCTCGCTCTACATG
AGGAATTTATGGTCCCGGCCACTAAAGCTTTGAACCTTCCAGCAAGGACAGTGACTGCTGACTCCAGA
ACAATATCTGATAGTCCTATGACAAACATAAATTATGGCACAGAATTCTCTCCTGTTGTGAATTCACA
AATACTACCACCTGAAGAACCCACAGATTTCAAACTGTCTACTGCTATTAAAACTACAGCCATGTCAA
AGAATATAAACCCAACCATGTCAAGCCAAATACAAGGCACAACCAATCAACATTCATCCACTGTCTTT
CCACTGCTACTTGGAGCAACTGAATTTCAGGACTCTGACCAGATGGGAAGAGGAAGAGAGCATTTCCA
AAGTAGACCCCCAATAACAGTAAGGACTATGATCAAAGATGTCAATGTCAAAATGCTTAGTAGCACCA
CCAACAAACTATTATTAGAGTCAGTAAATACCACAAATAGTCATCAGACATCTGTAAGAGAAGTGAGT
GAACCCAGGCACAATCACTTCTATTCTCACACTACTCAAATACTTAGCACCTCCACGTTCCCTTCAGA
TCCACACACAGCTGCTCATTCTCAGTTTCCGATCCCTAGAAATAGTACAGTTAACATCCCGCTGTTCA
GACGCTTTGGGAGGCAGAGGAAAATTGGCGGAAGGGGGCGGATTATCAGCCCATATAGAACTCCAGTT
CTGCGACGGCATAGATACAGCATTTTCAGGTCAACAACCAGAGGTTCTTCTGAAAAAAGCACTACTGC
ATTCTCAGCCACAGTGCTCAATGTGACATGTCTGTCCTGTCTTCCCAGGGAGAGGCTCACCACTGCCA
CAGCAGCATTGTCTTTTCCAAGTGCTGCTCCCATCACCTTCCCCAAAGCTGACATTGCTAGAGTCCCA
TCAGAAGAGTCTACAACTCTAGTCCAGAATCCACTATTACTACTTGAGAACAAACCCAGTGTAGAGAA
AACAACACCCACAATAAAATATTTCAGGACTGAAATTTCCCAAGTGACTCCAACTGGTGCAGTCATGA
CATATGCTCCAACATCCATACCCATGGAAAAAACTCACAAAGTAAACGCCAGTTACCCACGTGTGTCT
AGCACCAATGAAGCTAAAAGAGATTCAGTGATTACATCGTCACTTTCAGGTGCTATCACCAAGCCACC
AATGACTATTATAGCCAATTACAAGGTTTTCAAGAAGGAAATTCCCTGGCAACAGAACTTTGTAAATA
ACCATAACCCAAAAGGCAGATTAAGGAATCAACATAAAGTTAGTTTACAAAAAAGCACAGCTGTGATG
CTTCCTAAAACATCTCCTGCTTTACCACAGAGACAAAGTCTCCCCTCGCACCACACTACGACCAAAAC
ACACAATCCTGGAAGTCTTCCAACAAAGAAGGAGCTTCCCTTCCCACCCCTTAACCCTATGCTTCCTA
GTATTATAAGCAAAGACTCAAGTACAAAAAGCATCATATCAACGCAAACAGCAATACCAGCAACAACT
CCTACCTTCCCTGCATCTGTCATCACTTATGAAACCCAAACAGAGAGATCTAGAGCACAAACAATACA
AAGAGAACAGGAGCCTCAAAAGAAGAACAGGACTGACCCAAACATCTCTCCAGACCAGAGTTCTGGCT
TCACTACACCCACTGCTATGACACCTCCTGTTCTAACCACAGCCGAAACTTCAGTCAAGCCCAGTGTC
TCTGCATTCACTCATTCCCCACCAGAAAACACAACTGGGATTTCAAGCACAATCAGTTTTCATTCAAG
AACTCTTAATCTGACAGATGTGATTGAAGAACTAGCCCAAGCAAGTACTCAGACTTTGAAGAGCACAA
TTGCTTCTGAAACAACTTTGTCCAGCAAATCACACCAGAGTACCACAACTAGGAAAGCAATCATTAGA
CACTCAACCATACCACCATTCTTGAGCAGCAGTGCTACTCTAATGCCAGTTCCCATCTCCCCTCCCTT
TACTCAGAGAGCAGTTACTGACAACGTGGCGACTCCCATTTCCGGGCTTATGACAAATACAGTGGTCA
AGCTGCACGAATCCTCAAGGCACAATGCTAAACCACAGCAATTAGTAGCAGAGGTTGCAACATCCCCC
AAGGTTCACCCAAATGCCAAGTTCACAATTGGAACCACTCACTTCATCTACTCTAATCTGTTACATTC
TACTCCCATGCCAGCACTAACAACAGTTAAATCACAGAATTCTAAATTAACTCCATCTCCCTGGGCAG
AAAACCAATTTTGGCACAAACCATACTCAGAAATTGCTGAAAAAGGCAAAAAGCCAGAAGTAAGCATG
TTGGCTACTACAGGCCTGTCCGAGGCCACCACTCTTGTTTCAGATTGGGATGGACAGAAGAACACAAA
GAAGAGTGACTTTGATAAGAAACCAGTTCAAGAAGCAACAACTTCCAAACTCCTTCCCTTTGACTCTT
TGTCTAGGTATATATTTGAAAAGCCCAGGATAGTTGGAGGAAAAGCTGCAAGTTTTACTATTCCAGCT
AACTCAGATGCCTTTCTTCCCTGTGAAGCTGTTGGAAATCCCCTGCCCACCATTCATTGGACCAGAGT
CCCATCAGGTATGTCAGGACTTGATTTATCTAAGAGGAAACAGAATAGCAGGGTCCAGGTTCTCCCCA
ATGGTACCCTGTCCATCCAGAGGGTGGAAATTCAGGACCGCGGACAGTACTTGTGTTCCGCATCCAAT
ACGTACCAAAGAGATCACAGTTCATTCCGGAAGCACTGTGGAACTGAAGTGCAGAGCAGAAGGTAGGC
CAAGCCCTACAGTTACCTGGATTCTTGCAAACCAAACAGTTGTCTCAGAATCATCCCAGGGAAGTAGG
CAGGCTGTGGTGACGGTTGACGGAACATTGGTCCTCCACAATCTCAGTATTTATGACCGTGGCTTTTA
CAAATGTGTGGCCAGCAACCCAGGTGGCCAGGATTCACTGCTGGTTAAAATACAAGTCATTGCAGCAC
TGTACTGCAAAAGGAACTCCTCAGCCCAGCGTTTACTGGGTCCTCTCTGATGGCACTGAAGTGAAACC
ATTACAGTTTACCAATTCCAAGTTGTTCTTATTTTCAAATGGGACTTTGTATATAAGAAACCTAGCCT
CTTACAATGGAAGAGCGAGTGACCAGCCCCAGGATAGAAGCTGCATCCCAGAAAAGGACTGAAGTGAA
TTTTGGGGACAAATTACTACTGAACTGCTCAGCCACTGGGGAGCCCAAACCCCAAATAATGTGGAGGT
TACCATCCAAGGCTGTGGTCGACCAGCAGCATAGGGTGGGCAGCTGGATCCACGTCTACCCTAATGGA
TCCCTGTTTATTGGATCAGTAACAGAAAAAGACAGTGGTGTCTACTTGTGTGTGGCAAGAAACAAAAT
GGGGGATGATCTGATACTGATGCATGTTAGCCTAAGACTGAAACCTGCCAAAATTGACCACAAGCAGT
ATTTTAGAAAGCAAGTGCTCCATGGGAAAGATTTCCAAGTAGATTGCAAAGCTTCCGGCTCCCCAGTG
CCAGAGATATCTTGGAGTTTGCCTGATGGAACCATGATCAACAATGCAATGCAAGCCGATGACAGTGG
CCACAGGACTAGGAGATATACCCTTTTCAACAATGGAACTTTATACTTCAACAAAGTTGGGGTAGCGG
AGGAAGGAGATTATACTTGCTATGCCCAGAACACCCTAGGGAAAGATGAAATGAAGGTCCACTTAACA
GTTATAACAGCTGCTCCCCGGATAAGGCAGAGTAACAAAACCAACAAGAGAATCAAAGCTGGAGACAC
AGCTGTCCTTGACTGTGAGGTCACTGGGGATCCCAAACCAAAAATATTTTGGTTGCTGCCTTCCAATG
ACATGATTTCCTTCTCCATTGATAGGTACACATTTCATGCCAATGGGTCTTTGACCATCAACAAAGTG
AAACTGCTCGATTCTGGAGAGTACGTATGTGTAGCCCGAAATCCCAGTGGGGATGACACCAAAATGTA
CAAACTGGATGTGGTCTCTAAACCTCCATTAATCAATGGTCTGTATACAAATAGAACTGTTATTAAAG
CCACAGCTGTGAGACATTCCAAAAAACACTTTGACTGCAGAGCTGAAGGGACACCATCTCCTGAAGTC
ATGTGGATCATGCCAGACAATATTTTCCTCACAGCCCCATACTATGGAAGCAGAATCACAGTCCATAA
AAATGGAACCTTGGAAATTAGGAATGTGAGGCTTTCAGATTCAGCCGACTTTATCTGTGTGGCCCGAA
ATGAAGGTGGAGAGAGCGTGTTGGTAGTACAGTTAGAAGTACTGGAAATGCTGAGAAGACCGACATTT
AGAAATCCATTTAATGAAAAAATAGTTGCCCAGCTGGGAAAGTCCACAGCATTGAATTGCTCTGTTGA
TGGTAACCCACCACCTGAAATAATCTGGATTTTACCAAATGGCACACGATTTTCCAATGGACCACAAA
GTTATCAGTATCTGATAGCAAGCAATGGTTCTTTTATCATTTCTAAAACAACTCGGGAGGATGCAGGA
AAATATCGCTGTGCAGCTAGGAATAAAGTTGGCTATATTGAGAAATTAGTCATATTAGAAATTGGCCA
GAAGCCAGTTATTCTTACCTATGCACCAGGGACAGTAAAAGGCATCAGTGGAGAATCTCTATCACTGC
ATTGTGTGTCTGATGGAATCCCTAAGCCAAATATCAAATGGACTATGCCAAGTGGTTATGTAGTAGAC
AGGCCTCAAATTAATGGGAAATACATATTGCATGACAATGGCACCTTAGTCATTAAAGAAGCAACAGC
TTATGACAGAGGAAACTATATCTGTAAGGCTCAAAATAGTGTTGGTCATACACTGATTACTGTTCCAG
TAATGATTGTAGCCTACCCTCCCCGAATTACAAATCGTCCACCCAGGAGTATTGTCACCAGGACAGGG
GCAGCCTTTCAGCTCCACTGTGTGGCCTTGGGAGTTCCCAAGCCAGAAATCACATGGGAGATGCCTGA
CCACTCCCTTCTCTCAACGGCAAGTAAAGAGAGGACACATGGAAGTGAGCAGCTTCACTTACAAGGTA
CCCTAGTCATTCAGAATCCCCAAACCTCCGATTCTGGGATATACAAATGCACAGCAAAGAACCCACTT
GGTAGTGATTATGCAGCAACGTATATTCAAGTAATCTGACATGAAATAATAAAGTC

| | | | |
|---|---|---|---|
| NOV29ap, SNP13377641 of | SEQ ID NO:748 | 2617 aa | MW at 290200.2 kD |
| CG55776-01, Protein Sequence | SNP Pos: 2237 | | SNP Change: Asn to Asn |

MKVKGRGITCLLVSFAVICLVATPGGKACPRRCACYMPTEVHCTFRYLTSIPDSIPPNVERINLGYNS
LVRLMETDFSGLTKLELLMLHSNGIHTIPDKTFSDLQALQVRLMVLKMSYNKVRKLQKDTFYGLRSLT
RLHMDHNNIEFINPEVFYGLNFLRLVHLEGNQLTKLHPDTFVSLSYLQIFKISFIKFLYLSDNFLTSL
PQEMVSYMPDLDSLYLHGNPWTCDCHLKWLSDWIQEKPGIYIVLPDVIKCKKDRSPSSAQQCPLCMNP
RTSKGKPLAMVSAAAFQCAKPTIDSSLKSKSLTILEDSSSAFISPQGFMAPFGSLTLNMTDQSGNEAN
MVCSIQKPSRTSPIAFTEENDYIVLNTSFSTFLVCNIDYGHIQPVWQILALYSDSPLILERSHLLSET
PQLYYKYKQVAPKPEDIFTNIEADLRADPSWLMQDQISLQLNRTATTFSTLQIQYSSDAQITLPRAEM
RPVKHKWTMISRDNNTKLEHTVLVGGTVGLNCPGQGDPTPHVDWLLADGSKVRAPYVSEDGRILIDKS
GKLELQMADSFDTGVYHCISSNYDDADILTYRITVVEPLVEAYQENGIHHTVFIGETLDLPCHSTGTP
DASISWVIPGNNVLYQSSRDKKVLNNGTLRILQVTPKDQGYYRCVAANPSGVDFLIFQVSVKMKGQRP
LEHDGETEGSGLDESNPIAHLKEPPGAQLRTSALMEAEVGKHTSSTSKRHNYRELTLQRRGDSTHRRF
RENRRHFPPSARRIDPQHWAALLEKAKKNAMPDKRENTTVSPPPVVTQLPNIPGEEDDSSGMLALHEE
FMVPATKALNLPARTVTADSRTISDSPMTNINYGTEFSPVVNSQILPPEEPTDFKLSTAIKTTAMSKN
INPTMSSQIQGTTNQHSSTVFPLLLGATEFQDSDQMGRGREHFQSRPPITVRTMIKDVNVKMLSSTTN
KLLLESVNTTNSHQTSVREVSEPRHNHFYSHTTQILSTSTFPSDPHTAAHSQFPIPRNSTVNIPLFRR
FGRQRKIGGRGRIISPYRTPVLRPHRYSIFRSTTRGSSEKSTTAFSATVLNVTCLSCLPRERLTTATA
ALSFPSAAPITFPKADIARVPSEESTTLVQNPLLLLENKPSVEKTTPTIKYFRTEISQVTPTGAVMTY
APTSIPMEKTHKVNASYPRVSSTNEAKRDSVITSSLSGAITKPPMTIIAITRFSRRKIPWQQNFVNNH
NPKGRLRNQHKVSLQKSTAVMLPKTSPALPQRQSLPSHHTTTKTHNPGSLPTKKELPFPPLNPMLPSI
ISKDSSTKSIISTQTAIPATTPTFPASVITYETQTERSRAQTIQREQEPQKKNRTDPNISPDQSSGFT
TPTAMTPPVLTTAETSVKPSVSAFTHSPPENTTGISSTISFHSRTLNLTDVIEELAQASTQTLKSTIA
SETTLSSKSHQSTTTRKAIIRHSTIPPFLSSSATLMPVPISPPFTQRAVTDNVATPISGLMTNTVVKL
HESSRHNAKPQQLVAEVATSPKVHPNAKFTIGTTHFIYSNLLHSTPMPALTTVKSQNSKLTPSPWAEN
QFWHKPYSEIAEKGKKPEVSMLATTGLSEATTLVSDWDGQKNTKKSDFDKKPVQEATTSKLLPFDSLS
YIFEKPRIVGGKAASFTIPAIASDAFLPCEAVGNPLPTIHWTRVPSGMSGLDLSKRKQNSRVQVLPNG
TLSIQRVEIQDRGQYLCSASNLFGTDHLHVTLSVVSYPPRILERRTKEITVHSGSTVELKCRAEGRPS
PTVTWILANQTVVSESSQGSRQAVVTVDGTLVLHNLSIYDRGFYKCVASNPGGQDSLLVKIQVIAAPP
VILEQRRQVIVGTWGESLKLPCTAKGTPQPSVYWVLSDGTEVKPLQFTNSKLFLFSNGTLYIRNLASS
DRGTYECIATSSTGSERRVVMLTMEERVTSPRIEAASQKRTEVNFGDKLLLNCSATGEPKPQIMWRLP
SKAVVDQQHRVGSWIHVYPNGSLFIGSVTEKDSGVYLCVARNKMGDDLILMHVSLRLKPAKIDHKQYF
RKQVLHGKDFQVDCKASGSPVPEISWSLPDGTMINNAMQADDSGHRTRRYTLFNNGTLYFNKVGVAEE
GDYTCYAQNTLGKDEMKVHLTVITAAPRIRQSNKTNKRIKAGDTAVLDCEVTGDPKPKIFWLLPSNDM
ISFSIDRYTFHANGSLTINKVKLLDSGEYVCVARNPSGDDTKMYKLDVVSKPPLINGLYTNRTVIKAT
AVRHSKKHFDCRAEGTPSPEVMWIMPDNIFLTAPYYGSRITVHKNGTLEIRNVRLSDSADFICVARNE
GGESVLVVQLEVLEMLRRPTFRNPFNEKIVAQLGKSTALNCSVDGNPPPEIIWILPNGTRFSNGPQSY
QYLIASNGSFIISKTTREDAGKYRCAARNKVGYIEKLVILEIGQKPVILTYAPGTVKGISGESLSLHC
VSDGIPKPNIKWTMPSGYVVDRPQINGKYILHDNGTLVIKEATAYDRGNYICKAQNSVGHTLITVPVM
IVAYPPRITNRPPRSIVTRTGAAFQLHCVALGVPKPEITWEMPDHSLLSTASKERTHGSEQLHLQGTL
VIQNPQTSDSGIYKCTAKNPLGSDYAATYIQVI

| | | |
|---|---|---|
| NOV29aq, SNP13377640 | SEQ ID NO:749 | 7876 bP |
| of CG55776-01, | ORF Start: ATG at 6 | ORF Stop: TGA at 7857 |
| DNA Sequence | SNP Pos: 7218 | SNP Change: T to C |

TCAGGATGAAGGTAAAAGGCAGAGGAATCACCTGCTTGCTGGTCTCCTTTGCTGTGATCTGCCTGGTC
GCCACCCCTGGGGGCAAGGCCTGTCCTCGCCGCTGTGCCTGTTATATGCCTACGGAGGTACACTGCAC
ATTTCGGTACCTGACTTCCATCCCAGACAGCATCCCGCCCAATGTGGAACGCATCAATTTAGGGTACA
ACAGCTTGGTTAGATTGATGGAAACAGATTTTTCTGGCCTGACCAAACTGGAGTTACTCATGCTTCAC
AGCAATGGCATTCACACAATCCCTGACAAGACCTTCTCAGATTTGCAGGCCTTGCAGGTGAGACTGAT
GGTCTTAAAAATGAGCTATAATAAAGTCCGAAAACTTCAGAAAGATACTTTTTATGGCCTCAGGAGCT
TGACACGATTGCACATGGACCACAACAATATTGAGTTTATAAACCCAGAGGTTTTTTATGGGCTCAAC
TTTCTCCGCCTGGTGCACTTGGAAGGAAATCAGCTCACTAAGCTCCACCCAGATACATTTGTCTCTTT
GAGCTACCTCCAGATATTTAAAATCTCTTTCATTAAGTTCCTATACTTGTCTGATAACTTCCTGACCT
CCCTCCCTCAAGAGATGGTCTCCTATATGCCTGACCTAGACAGCCTTTACCTGCATGGAAACCCATGG
ACCTGTGATTGCCATTTAAAGTGGTTGTCTGACTGGATACAGGAGAAGCCAGGTATCTATATTGTNTT
ACCAGATGTAATAAAATGCAAAAAAGATAGAAGTCCCTCTAGTGCTCAGCAGTGTCCACTTTGCATGA
ACCCTAGGACTTCTAAAGGCAAGCCGTTAGCTATGGTCTCAGCTGCAGCTTTCCAGTGTGCCAAGCCA
ACCATTGACTCATCCCTGAAATCAAAGAGCCTGACTATTCTGGAAGACAGTAGTTCTGCTTTCATCTC
TCCCCAAGGTTTCATGGCACCCTTTGGCTCCCTCACTTTGAATATGACAGATCAGTCTGGAAATGAAG
CTAACATGGTCTGCAGTATTCAAAAGCCCTCAAGGACATCACCCATTGCATTCACTGAAGAAAATGAC
TACATCGTGCTAAATACTTCATTTTCAACATTTTTGGTGTGCAACATAGATTACGGTCACATTCAGCC
AGTGTGGCAAATTTTGGCTTTGTACAGTGATTCTCCTCTGATACTAGAAAGGAGCCACTTGCTTAGTG
AAACACCGCAGCTCTATTACAAATATAAACAGGTGGCTCCTAAGCCTGAAGACATTTTTACCAACATA
GAGGCAGATCTCAGAGCAGATCCCTCTTGGTTAATGCAAGACCAAATTTCCTTGCAGCTGAACAGAAC
TGCCACCACATTCAGTACATTACAGATCCAGTACTCCAGTGATGCTCAAATCACTTTACCAAGAGCAG
AGATGAGGCCAGTGAAACACAAATGGACTATGATTTCAAGGGATAACAATACTAAGCTGGAACATACT
GCTTCTAGCTGATGGAAGTAAAGTGAGAGCCCCTTATGTCAGTGAGGATGGACGGATCCTAATAGACA
AAAGTGGAAAATTGGAACTCCAGATGGCTGATAGTTTTGACACAGGCGTATATCACTGTATAAGCAGC
AATTATGATGATGCAGATATTCTCACCTATAGGATAACTGTGGTAGAACCTTTGGTCGAAGCCTATCA
GGAAAATGGGATTCATCACACAGTTTTCATTGGTGAAACACTTGATCTTCCATGCCATTCTACTGGTA
TCCCAGATGCCTCTATTAGCTGGGTTATTCCAGGAAACAATGTGCTCTATCAGTCATCAAGAGACAAG
AAAGTTCTAAACAATGGCACATTAAGAATATTACAGGTCACCCCGAAAGACCAAGGTTATTATCGCTG
TGTGGCAGCCAACCCATCAGGGGTTGATTTTTTGATTTTCCAAGTTTCAGTCAAGATGAAAGGACAAA
AAGGAGCCACCAGGTGCACAACTCCGTACATCTGCTCTGATGGAGGCTGAGGTTGGAAAACACACCTC
AAGCACAAGTAAGAGGCACAACTATCGGGAATTAACACTCCAGCGACGTGGAGATTCAACACATCGAC
GTTTTAGGGAGAATAGGAGGCATTTCCCTCCCTCTGCTAGGAGAATTGACCCACAACATTGGGCGGCA
CTGTTGGAGAAAGCTAAAAAGAATGCTATGCCAGACAAGCGAGAAAATACCACAGTGAGCCCACCCCC
AGTGGTCACCCAACTCCCAAACATACCTGGTGAAGAAGACGATTCCTCAGGCATGCTCGCTCTACATG
AGGAATTTATGGTCCCGGCCACTAAAGCTTTGAACCTTCCAGCAAGGACAGTGACTGCTGACTCCAGA
ACAATATCTGATAGTCCTATGACAAACATAAATTATGGCACAGAATTCTCTCCTGTTGTGAATTCACA
AATACTACCACCTGAAGAACCCACAGATTTCAAACTGTCTACTGCTATTAAAACTACAGCCATGTCAA
AGAATATAAACCCAACCATGTCAAGCCAAATACAAGGCACAACCAATCAACATTCATCCACTGTCTTT
CCACTGCTACTTGGAGCAACTGAATTTCAGGACTCTGACCAGATGGGAAGAGGAAGAGAGCATTTCCA
AAGTAGACCCCCAATAACAGTAAGGACTATGATCAAAGATGTCAATGTCAAAATGCTTAGTAGCACCA
CCAACAAACTATTATTAGAGTCAGTAAATACCACAAATAGTCATCAGACATCTGTAAGAGAAGTGAGT
GAACCCAGGCACAATCACTTCTATTCTCACACTACTCAAATACTTAGCACCTCCACGTTCCCTTCAGA
TCCACACACAGCTGCTCATTCTCAGTTTCCGATCCCTAGAAATAGTACAGTTAACATCCCGCTGTTCA
GACGCTTTGGGAGGCAGAGGAAAATTGGCGGAAGGGGGCGGATTATCAGCCCATATAGAACTCCAGTT
CTGCGACGGCATAGATACAGCATTTTCAGGTCAACAACCAGAGGTTCTTCTGAAAAAAGCACTACTGC
ATTCTCAGCCACAGTGCTCAATGTGACATGTCTGTCCTGTCTTCCCAGGGAGAGGCTCACCACTGCCA
CAGCAGCATTGTCTTTTCCAAGTGCTGCTCCCATCACCTTCCCCAAAGCTGACATTGCTAGAGTCCCA
TCAGAAGAGTCTACAACTCTAGTCCAGAATCCACTATTACTACTTGAGAACAAACCCAGTGTAGAGAA
AACAACACCCACAATAAAATATTTCAGGACTGAAATTTCCCAAGTGACTCCAACTGGTGCAGTCATGA
CATATGCTCCAACATCCATACCCATGGAAAAAACTCACAAAGTAAACGCCACTTACCCACGTGTGTCT
AGCACCAATGAAGCTAAAAGAGATTCAGTGATTACATCGTCACTTTCAGGTGCTATCACCAAGCCACC
AATGACTATTATAGCCATTACAAGGTTTTCAAGAAGGAAAATTCCCTGGCAACAGAACTTTGTAAATA
ACCATAACCCAAAAGGCAGATTAAGGAATCAACATAAAGTTAGTTTACAAAAAAGCACAGCTGTGATG
ACACAATCCTGGAAGTCTTCCAACAAAGAAGGAGCTTCCCTTCCCACCCCTTAACCCTATGCTTCCTA
GTATTATAAGCAAAGACTCAAGTACAAAAAGCATCATATCAACGCAAACAGCAATACCAGCAACAACT
CCTACCTTCCCTGCATCTGTCATCACTTATGAAACCCAAACAGAGAGATCTAGAGCACAAACAATACA
AAGAGAACAGGAGCCTCAAAAGAAGAACAGGACTGACCCAAACATCTCTCCAGACCAGAGTTCTGGCT
TCACTACACCCACTGCTATGACACCTCCTGTTCTAACCACAGCCGAAACTTCAGTCAAGCCCAGTGTC
TCTGCATTCACTCATTCCCCACCAGAAAACACAACTGGGATTTCAAGCACAATCAGTTTTCATTCAAG
AACTCTTAATCTGACAGATGTGATTGAAGAACTAGCCCAAGCAAGTACTCAGACTTTGAAGAGCACAA
TTGCTTCTGAAACAACTTTGTCCAGCAAATCACACCAGAGTACCACAACTAGGAAAGCAATCATTAGA
CACTCAACCATACCACCATTCTTGAGCAGCAGTGCTACTCTAATGCCAGTTCCCATCTCCCCTCCCTT
TACTCAGAGAGCAGTTACTGACAACGTGGCGACTCCCATTTCCGGGCTTATGACAAATACAGTGGTCA
AGCTGCACGAATCCTCAAGGCACAATGCTAACCACAGCAATTAGTAGCAGAGGTTGCAAACATCCCCC
AAGGTTCACCCAAATGCCAAGTTCACAATTGGAACCACTCACTTCATCTACTCTAATCTGTTACATTC
TACTCCCATGCCAGCACTAACAACAGTTAAATCACAGAATTCTAAATTAACTCCATCTCCCTGGGCAG
AAAACCAATTTTGGCACAAACCATACTCAGAAATTGCTGAAAAAGGCAAAAAGCCAGAAGTAAGCATG
TTGGCTACTACAGGCCTGTCCGAGGCCACCACTCTTGTTTCAGATTGGGATGGACAGAAGAACACAAA
GAAGAGTGACTTTGATAAGAAACCAGTTCAAGAAGCAACAACTTCCAAACTCCTTCCCTTTGACTCTT
TGTCTAGGTATATATTTGAAAAGCCCAGGATAGTTGGAGGAAAAGCTGCAAGTTTTACTATTCCAGCT
AACTCAGATGCCTTTCTTCCCTGTGAAGCTGTTGGAAATCCCCTGCCCACCATTCATTGGACCAGAGT
CCCATCAGGTATGTCAGGACTTGATTTATCTAAGAGGAAACAGAATAGCAGGGTCCAGGTTCTCCCCA
ATGGTACCCTGTCCATCCAGAGGGTGGAAATTCAGGACCGCGGACAGTACTTGTGTTCCGCATCCAAT
CTGTTTGGCACAGACCACCTTCATGTCACCTTGTCTGTGGTTTCCTATCCTCCCAGGATCCTGGAGAG
ACGTACCAAAGAGATCACAGTTCATTCCGGAAGCACTGTGGAACTGAAGTGCAGAGCAGAAGGTAGGC
CAAGCCCTACAGTTACCTGGATTCTTGCAAACCAAACAGTTGTCTCAGAATCATCCCAGGGAAGTAGG
CAGGCTGTGGTGACGGTTGACGGAACATTGGTCCTCCACAATCTCAGTATTTATGACCGTGGCTTTTA
CAAATGTGTGGCCAGCAACCCAGGTGGCCAGGATTCACTGCTGGTTAAAATACAAGTCATTGCAGCAC
CACCTGTTATTCTAGAGCAAAGGAGGCAAGTCATTGTAGGCACTTGGGGTGAAAGTTTAAAACTGCCC
TGTACTGCAAAAGGAACTCCTCAGCCCAGCGTTTACTGGGTCCTCTCTGATGGCACTGAAGTGAAACC
ATTACAGTTTACCAATTCCAAGTTGTTCTTATTTTCAAATGGGACTTTGTATATAAGAAACCTAGCCT
CTTCAGACAGGGGCACTTATGAATGCATTGCTACCAGTTCCACTGGTTCGGAGCGAAGAGTAGTAATG
CTTACAATGGAAGAGCGAGTGACCAGCCCCAGGATAGAAGCTGCATCCCAGAAAAGGACTGAAGTGAA
TTTTGGGGACAAATTACTACTGAACTGCTCAGCCACTGGGGAGCCCAAACCCCAAATAATGTGGAGGT
TACCATCCAAGGCTGTGGTCGACCAGCAGCATAGGGTGGGCAGCTGGATCCACGTCTACCCTAATGGA
TCCCTGTTTATTGGATCAGTAACAGAAAAAGACAGTGGTGTCTACTTGTGTGTGGCAAGAAACAAAAT
GGGGGATGATCTGATACTGATGCATGTTAGCCTAAGACTGAAACCTGCCAAAATTGACCACAAGCAGT
ATTTTAGAAAGCAAGTGCTCCATGGGAAAGATTTCCAAGTAGATTGCAAAGCTTCCGGCTCCCCAGTG
CCAGAGATATCTTGGAGTTTGCCTGATGGAACCATGATCAACAATGCAATGCAAGCCGATGACAGTGG
CCACAGGACTAGGAGATATACCCTTTTCAACAATGGAACTTTATACTTCAACAAAGTTGGGGTAGCGG
AGGAAGGAGATTATACTTGCTATGCCCAGAACACCCTAGGGAAAGATGAAATGAAGGTCCACTTAACA
GTTATAACAGCTGCTCCCCGGATAAGGCAGAGTAACAAAACCAACAAGAGAATCAAAGCTGGAGACAC
ACATGATTTCCTTCTCCATTGATAGGTACACATTTCATGCCAATGGGTCTTTGACCATCAACAAAGTG
AAACTGCTCGATTCTGGAGAGTACGTATGTGTAGCCCGAAATCCCAGTGGGGATGACACCAAAATGTA
CAAACTGGATGTGGTCTCTAAACCTCCATTAATCAATGGTCTGTATACAAACAGAACTGTTATTAAAG
CCACAGCTGTGAGACATTCCAAAAAACACTTTGACTGCAGAGCTGAAGGGACACCATCTCCTGAAGTC
AAATGGAACCTTGGAAATTAGGAATGTGAGGCTTTCAGATTCAGCCGACTTTATCTGTGTGGCCCGAA
AGAAATCCATTTAATGAAAAAATAGTTGCCCAGCTGGGAAAGTCCACAGCATTGAATTGCTCTGTTGA
TGGTAACCCACCACCTGAAATAATCTGGATTTTACCAAATGGCACACGATTTTCCAATGGACCACAAA
AAATATCGCCGTGCAGCTAGGAATAAAGTTGGCTATATTGAGAAATTAGTCATATTAGAAATTGGCCA
GAAGCCAGTTATTCTTACCTATGCACCAGGGACAGTAAAAGGCATCAGTGGAGAATCTCTATCACTGC
ATTGTGTGTCTGATGGAATCCCTAAGCCAAATATCAAATGGACTATGCCAAGTGGTTATGTAGTAGAC
AGGCCTCAAATTAATGGGAAATACATATTGCATGACAATGGCACCTTAGTCATTAAAGAAGCAACAGC
TTATGACAGAGGAAACTATATCTGTAAGGCTCAAAATAGTGTTGGTCATACACTGATTACTGTTCCAG
TAATGATTGTAGCCTACCCTCCCCGAATTACAAATCGTCCACCCAGGAGTATTGTCACCAGGACAGGG
GCAGCCTTTCAGCTCCACTGTGTGGCCTTGGGAGTTCCCAAGCCAGAAATCACATGGGAGATGCCTGA
CCACTCCCTTCTCTCAACGGCAAGTAAAGAGAGGACACATGGAAGTGAGCAGCTTCACTTACAAGGTA
CCCTAGTCATTCAGAATCCCCAAACCTCCGATTCTGGGATATACAAATGCACAGCAAAGAACCCACTT
GGTAGTGATTATGCAGCAACGTATATTCAAGTAATCTGACATGAAATAATAAAGTC

| | | | |
|---|---|---|---|
| NOV29aq, SNP13377640 of | SEQ ID NO:750 | 2617 aa | MW at 290253.2 kD |
| CG55776-01, Protein Sequence | SNP Pos: 2405 | | SNP Change: Cys to Arg |

MKVKGRGITCLLVSFAVICLVATPGGKACPRRCACYMPTEVHCTFRYLTSIPDSIPPNVERINLGYNS
LVRLMETDFSGLTKLELLMLHSNGIHTIPDKTFSDLQALQVRLMVLKMSYNKVRKLQKDTFYGLRSLT
RLHMDHNNIEFINPEVFYGLNFLRLVHLEGNQLTKLHPDTFVSLSYLQIFKISFIKFLYLSDNFLTSL
PQEMKTSYMPDLDSLYLHGNPWTCDCHLKWLSDWIQEKPGIYIVLPDVIKCKKDRSPSSAQQCPLCAP
RTSKGKPLAMVSAAAFQCAKPTIDSSLKSKSLTILEDSSSAFISPQGFMAPFGSLTLNMTDQSGNEAN
MVCSIQKPSRTSPIAFTEENDYIVLNTSFSTFLVCNIDYGHIQPVWQILALYSDSPLILERSHLLSET
PQLYYKYKQVAPKPEDIFTNIEADLRADPSWLMQDQISLQLNRTATTFSTLQIQYSSDAQITLPEAEM
RPVKHKWTMISRDNNTKLEHTVLVGGTVGLNCPGQGDPTPNVDWLLADGSKVRAPYVSEDGRILIDKS
GKLELQMADSFDTGVYHCISSNYDDADILTYRITVVEPLVEAYQENGIHHTVFIGETLDLPCHSTGIP
DASISWVIPGNNVLYQSSRDKKVLNNGTLRILQVTPKDQGYYRCVAANPSGVDFLIFQVSVKMKGQRP
LEHDGETEGSGLDESNPIAHLKEPPGAQLRTSALMEAEVGKHTSSTSKRHNYRELTLQRRGDSTHRRF
RENRRHFPPSARRIDPQHWAALLEKAKKNAMPDKRENTTVSPPPVVTQLPNIPGEEDDSSGMLALHEE
FMVPATKALNLPARTVTADSRTISDSPMTNINYGTEFSPVVNSQILPPEEPTDFKLSTAIKTTAMSKN
INPTMSSQIQGTTNQHSSTVFPLLLGATEFQDSDQMGRGREHFQSRPPITVRTMIKDVNVKMLSSTTN
KLLLESVNTTNSHQTSVREVSEPRHNHFYSHTTQILSTSTFPSDPHTAAHSQFPIPRNSTVNIPLFRR
FGRQRKIGGRGRIISPYRTPVLRRHRYSIFRSTTRGSSEKSTTAFSATVLNVTCLSCLPRERLTTATA
ALSFPSAAPITFPKADIARVPSEESTTLVQNPLLLLENKPSVEKTTPTIKYFRTEISQVTPTGAVMTY
APTSIPMEKTHKVNASYPRVSSTNEAKRDSVITSSLSGAITKPPMTIIAITRFSRRKIPWQQNFVNNH
NPKGRLRNQHKVSLQKSTAVMLPKTSPALPQRQSLPSHHTTTKTHNPGSLPTKKELPFPPLNPMLPSI
ISKDSSTKSIISTQTAIPATTPTFPASVITYETQTERSRAQTIQREQEPQKKNRTDPNISPDQSSGFT
TPTAMTPPVLTTAETSVKPSVSAFTHSPPENTTGISSTISFHSRTLNLTDVIEELAQASTQTLKSTIA
SETTLSSKSHQSTTTRKAIIRHSTIPPFLSSSATLMPVPISPPFTQRAVTDNVATPISGLMTNTVVKL
HESSRHNAKPQQLVAEVATSPKVHPNAKFTIGTTHFIYSNLLHSTPMPALTTVKSQNSKLTPSPWAEN
QFWHKPYSEIAEKGKKPEVSMLATTGLSEATTLVSDWDGQKNTKKSDFDKKPVQEATTSKLLPFDSLS
RYIFEKPRIVGGKAASFTIPANSDAFLPCEAVGNPLPTIHWTRVPSGMSGLDLSKRKQNSRVQVLPNG
TLSIQRVEIQDRGQYLCSASNLFGTDHLHVTLSVVSYPPRILERRTKEITVHSGSTVELKCRAEGRPS
PTVTWILANQTVVSESSQGSRQAVVTVDGTLVLHNLSIYDRGFYKCVASNPGGQDSLLVKIQVIAAPP
VILEQRRQVIVGTWGESLKLPCTAKGTPQPSVYWVLSDGTEVKPLQFTNSKLFLFSNGTLYIRNLASS
DRGTYECIATSSTGSERRVVMLTMEERVTSPRIEAASQKRTEVNFGDKLLLNCSATGEPKPQIMWRLP
SKAVVDQQHRVGSWIHVYPNGSLFIGSVTEKDSGVYLCVARNKMGDDLILMHVSLRLKPAKIDHKQYF
RKQVLHGKDFQVDCKASGSPVPEISWSLPDGTMINNAMQADDSGHRTRRYTLFNNGTLYFNKVGVAEE
GDYTCYAQNTLGKDEMKVHLTVITAAPRIRQSNKTNKRIKAGDTAVLDCEVTGDPKPKIFWLLPSNDM
ISFSIDRYTFHANGSLTINKVKLLDSGEYVCVARNPSGDDTKMYKLDVVSKPPLINGLYTNRTVIKAT
AVRHSKKHFDCRAEGTPSPEVMWIMPDNIFLTAPYYGSRITVHKNGTLEIRNVRLSDSADFICVARNE
QYLIASNGSFIISKTTREDAGKYPRAARNKVGYIEKLVILEIGQKPVILTYAPGTVKGISGESLSLHC
VSDGIPKPNIKWTMPSGYVVDRPQINGKYILHDNGTLVIKEATAYDRGNYICKAQNSVGHTLITVPVM
IVAYPPRITNRPPRSIVTRTGAAFQLHCVALGVPKPEITWEMPDHSLLSTASKERTHGSEQLHLQGTL
VIQNPQTSDSGIYKCTAKNPLGSDYAATYIQVI

A ClustalW comparison of the above protein sequences yields the following sequence alignment shown in Table 29B.

TABLE 29B
Comparison of the NOV29 protein sequences.

| | |
|---|---|
| NOV29a | MKVKGRGITCLLVSFAVICLVATPGGKACPRRCACYMPTEVHCTFRYLTSIPDSIPPNVE |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | MKVKGRGITCLLVSFAVICLVATPGGKACPRRCACYMPTEVHCTFRYLTSIPDSIPPNVE |
| NOV29aa | MKVKGRGITCLLVSFAVICLVATPGGKACPRRCACYMPTEVHCTFRYLTSIPDSIPPNVE |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | RINLGYNSLVRLMETDFSGLTKLELLMLHSNGIHTIPDKTFSDLQALQVRLMVLKMSYNK |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | RINLGYNSLVRLMETDFSGLTKLELLMLHSNGIHTIPDKTFSDLQALQVRLMVLKMSYNK |
| NOV29aa | RINLGYNSLVRLMETDFSGLTKLELLMLHSNGIHTIPDKTFSDLQALQVRLMVLKMSYNK |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | VRKLQKDTFYGLRSLTRLHNDHNNIEFINPEVFYGLNFLRLVHLEGNQLTKLHPDTFVSL |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | VRKLQKDTFYGLRSLTRLHMDHNNIEFINPEVFYGLNFLRLVHLEGNQLTKLHPDTFVSL |
| NOV29aa | VRKLQKDTFYGLRSLTRLHMDHNNIEFINPEVFYGLNFLRLVHLEGNQLTKLHPDTFVSL |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | SYLQIFKISFIKFLYLSDNFLTSLPQEMVSYMPDLDSLYLHGNPWTCDCHLKWLSDWIQE |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | SYLQIFKISFIKFLYLSDNFLTSLPQEMVSYMPDLDSLYLHGNPWTCDCHLKWLSDWIQE |
| NOV29aa | SYLQIFKISFIKFLYLSDNFLTSLPQEMVSYMPDLDSLYLHGNPWTCDCHLKWLSDWIQE |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | KPGIYIVLPDVIKCKKDRSPSSAQQCPLCMNPRTSKGKPLAMVSAAAFQCAKPTIDSSLK |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | KPGIYIVLPDVIKCKKDRSPSSAQQCPLCMNPRTSKGKPLAMVSAAAFQCAKPTIDSSLK |
| NOV29aa | KPGIYIVLPDVIKCKKDRSPSSAQQCPLCMNPRTSKGKPLAMVSAAAFQCAKPTIDSSLK |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | SKSLTILEDSSSAFISPQGFMAPFGSLTLNMTDQSGNEANMVCSIQKPSRTSPIAFTEEN |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | SKSLTILEDSSSAFISPQGFMAPFGSLTLNMTDQSGNEANMVCSIQKPSRTSPIAFTEEN |
| NOV29aa | SKSLTILEDSSSAFISPQGFMAPFGSLTLNMTDQSGNEANMVCSIQKPSRTSPIAFTEEN |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | DYIVLNTSFSTFLVCNIDYGHIQPVWQILALYSDSPLILERSHLLSETPQLYYKYKQVAP |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | DYIVLNTSFSTFLVCNIDYGHTQPVWQILALYSDSPLILERSHLLSETPQLYYKYKQVAP |
| NOV29aa | DYIVLNTSFSTFLVCNIDYGHTQPVWQILALYSDSPLILERSHLLSETPQLYYKYKQVAP |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | KPEDIFTNIEADLRADPSWLMQDQISLQLNRTATTFSTLQIQYSSDAQITLPRAEMRPVK |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | KPEDIFTNIEADLRADPSWLMQDQISLQLNRTATTFSTLQIQYSSDAQITLPPAENRPVK |
| NOV29aa | KPEDIFTNIEADLRADPSWLMQDQISLQLNRTATTFSTLQIQYSSDAQITLPEAENRPVK |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | HKWTMISRDNNTKLEHTVLVGGTVGLNCPGQGDPTPHVDWLLADGSKVRAPYVSEDGRIL |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | HKWTMISRDNNTKLEHTVLVGGTVGLNCPGQGDPTPHVDWLLADGSKVRAPYVSEDGRIL |
| NOV29aa | HKWTMISRDNNTKLEHTVLVGGTVGLNCPGQGDPTPHVDWLLADGSKVRAPYVSEDGRIL |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | IDKSGKLELQMADSFDTGVYHCISSNYDDADILTYRITVVEPLVEAYQENGIHHTVFIGE |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | IDKSGKLELQMADSFDTGVYHCISSNYDDADILTYRITVVEPLVEAYQENGIHHTVFIGE |
| NOV29aa | IDKSGKLELQMADSFDTGVYHCISSNYDDADILTYRITVVEPLVEAYQENGIHHTVFIGE |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | TLDLPCHSTGIPDASISWVIPGNNVLYQSSRDKKVLNNGTLRILQVTPKDQGYYRCVAAN |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | TLDLPCHSTGIPDASISWVIPGNNVLYQSSRDKKVLNNGTLRILQVTPKDQGYYRCVAAN |
| NOV29aa | TLDLPCHSTGIPDASISWVIPGNNVLYQSSRDKKVLNNGTLRILQVTPKDQGYYRCVAAN |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | PSGVDFLIFQVSVKMKGQRPLEHDGETEGSGLDESNPIAHLKEPPGAQLRTSALMEAEVG |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | PSGVDFLIFQVSVKMKGQRPLEHDGETEGSGLDESNPIAHLKEPPGAQLRTSALMEAEVG |
| NOV29aa | PSGVDFLIFQVSVKMKGQRPLEHDGETEGSGLDESNPIAHLKEPPGAQLRTSALMEAEVG |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | KHTSSTSKRHNYRELTLQRRGDSTHRRFRENRRHFPPSARRIDPQHWAALLEKAKKNAMP |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | KHTSSTSKRHNYRELTLQRRGDSTHRRFRENRRHFPPSARRIDPQHWAALLEKAKKNAMP |
| NOV29aa | KHTSSTSKRHNYRELTLQRRGDSTHRRFRENRRHFPPSARRIDPQHWAALLEKAKKNAMP |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | DKRENTTVSPPPVVTQLPNIPGEEDDSSGMLALHEEFMVPATKALNLPARTVTADSRTIS |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | DKRENTTVSPPPVVTQLPNIPGEEDDSSGMLALHEEFMVPATKALNLPARTVTADSRTIS |
| NOV29aa | DKRENTTVSPPPVVTQLPNIPGEEDDSSGMLALHEEFMVPATKALNLPARTVTADSRTIS |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | DSPMTNINYGTEFSPVVNSQILPPEEPTDFKLSTAIKTTAMSKNINPTMSSQIQGTTNQH |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | DSPMTNINYGTEFSPVVNSQILPPEEPTDFKLSTAIKTTAMSKNINPTMSSQIQGTTNQH |
| NOV29aa | DSPMTNINYGTEFSPVVNSQILPPEEPTDFKLSTAIKTTAMSKNINPTMSSQIQGTTNQH |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | SSTVFPLLLGATEFQDSDQMGRGREHFQSRPPITVRTMIKDVNVKMLSSTTNKLLLESVN |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | SSTVFPLLLGATEFQDSDQMGRGREHFQSRPPITVRTMIKDVNVKNLSSTTNKLLLESVN |
| NOV29aa | SSTVFPLLLGATEFQDSDQMGRGREHFQSRPPITVRTMIKDVNVKNLSSTTNKLLLESVN |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | TTNSHQTSVREVSEPRHNHFYSHTTQILSTSTFPSDPHTAAHSQFPIPRNSTVNIPLFRR |
| NOV29b | ------------------------------------------------------------ |
| KOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | TTNSHQTSVREVSEPRHNHFYSHTTQILSTSTFPSDPHTAAHSQFPIPRNSTVNIPLFRR |
| NOV29aa | TTNSHQTSVREVSEPRENHFYSHTTQILSTSTFPSDPHTAAHSQFPIPRNSTVNIPLFRR |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | FGRQRKIGGRGRIISPYRTPVLRRHRYSIFRSTTRGSSEKSTTAFSATVLNVTCLSCLPR |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | FGRQRKIGGRGRIISPYRTPVLRRHRYSIFRSTTRGSSEKSTTAFSATVLNVTCLSCLPR |
| NOV29aa | FGRQRKIGGRGRIISPYRYPVLRRHRYSIFRSTTRGSSEKSTTAFSATVLNVTCLSCLPR |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | ERLTTATAALSFPSAAPITFPKADIARVPSEESTTLVQNPLLLLENKPSVEKTTPTIKYF |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | ERLTTATAALSFPSAAPITFPKADIARVPSEESTTLVQNPLLLLENKPSVEKTTPTIKYF |
| NOV29aa | ERLTTATAALSFPSAAPITFPKADIARVPSEESTTLVQNPLLLLENKPSVEKTTPTIKYF |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | RTEISQVTPTGAVMTYAPTSIPMEKTHKVNASYPRVSSTNEAKRDSVITSSLSGAITKPP |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | RTEISQVTPTGAVMTYAPTSIPMEKTHKVNASYPRVSSTNEAKRDSVITSSLSGAITKPP |
| NOV29aa | RTEISQVTPTGAVMTYAPTSIPMEKTHKVNASYPRVSSTNEAKRDSVITSSLSGAITKPP |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | MTIIAITRFSRRKIPWQQNFVNNHNPKGRLRNQHKVSLQKSTAVMLPKTSPALPQRQSLP |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | MTIIAITRFSRRKIPWQQNFVNNHNPKGRLRNQHKVSLQKSTAVMLPKTSPALPQRQSLP |
| NOV29aa | MTIIAITRFSRRKIPWQQNFVNNHNPKGRLRNQHKVSLQKSTAVMLPKTSPALPQRQSLP |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | SHHTTTKTHNPGSLPTKKELPFPPLNPMLPSIISKDSSTKSIISTQTAIPATTPTFPASV |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | SHHTTTKTHNPGSLPTKKELPFPPLNPMLPSIISKDSSTKSIISTQTAIPATTPTFPASV |
| NOV29aa | SHHTTTKTHNPGSLPTKKELPFPPLNPMLPSIISKDSSTKSIISTQTAIPATTPTFPASV |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | ITYETQTERSRAQTIQREQEPQKKNRTDPNISPDQSSGFTTPTAMTPPVLTTAETSVKPS |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | ITYETQTERSEAQTIQREQEPQKKNRTDPNISPDQSSGFTTPTAMTPPVLTTAETSVKPS |
| NOV29aa | ITYETQTERSRAQTIQREQEPQKKNRTDPNISPDQSSGFTTPTAMTPPVLTTAETSVKPS |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | VSAFTHSPPENTTGISSTISFHSRTLNLTDVIEELAQASTQTLKSTIASETTLSSKSHQS |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | VSAFTHSPPENTTGISSTISFHSRTLNLTDVIEELAQASTQTLKSTIASETTLSSKSHQS |
| NOV29aa | VSAFTHSPPENTTGISSTISFHSRTLNLTDVIEELAQASTQTLKSTIASETTLSSKSHQS |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | TTTRKAIIRHSTIPPFLSSSATLMPVPISPPFTQPAVTDNVATPISGLMTNTVVKLHESS |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | TTTRKAIIRHSTIPPFLSSSATLMPVPISPPFTQRAVTDNVATPISGLMTNTVVKLHESS |
| NOV29aa | TTTRKAIIRHSTIPPFLSSSATLMPVPISPPFTQRAVTDNVATPISGLMTNTVVKLHESS |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | RHNAKPQQLVAEVATSFKVHPNAKFTIGTTHFIYSNLLHSTPMPALTTVKSQNSKLTPSP |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | RHNAKPQQLVAEVATSPKVHPNAKFTIGTTHFIYSNLLHSTPMPALTTVKSQNSKLTPSP |
| NOV29aa | RHNAKPQQLVAEVATSFKVHPNAKFTIGTTHFIYSNLLHSTPMPALTTVKSQNSKLTPSP |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | WAENQFWHKPYSEIAEKGKKPEVSMLATTGLSEATTLVSDWDGQKNTKKSDFDKKPVQEA |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------------------------------------ |
| NOV29y | ------------------------------------------------------------ |
| NOV29z | WAENQFWHKPYSEIAEKGKKPEVSMLATTGLSEATTLVSDWDGQKNTKKSDFDKKPVQEA |
| NOV29aa | WAENQFWHKPYSEIAEKGKKPEVSMLATTGLSEATTLVSDWDGQKNTKKSDFDKKPVQEA |
| NOV29ab | ------------------------------------------------------------ |
| NOV29ac | ------------------------------------------------------------ |
| NOV29ad | ------------------------------------------------------------ |
| NOV29ae | ------------------------------------------------------------ |
| NOV29af | ------------------------------------------------------------ |
| NOV29ag | ------------------------------------------------------------ |
| NOV29a | TTSKLLPFDSLSRYIFEKPRIVGGKAASFTIPANSDAFLPCEAVGNPLPTIHWTRVPSGM |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ------------------------------TLENSDAFLPCEAVGNPLPTIHWTRVPS-- |
| NOV29y | ------------------------------TRSNSDAFLPCEAVGNPLPTIHWTRVPS-- |
| NOV29z | TTSKLLPFDSLSRYIFEKPRIVGGKAASFTIPANSDAFLPCEAVGNPLPTIHWTRVPSGM |
| NOV29aa | TTSKLLPFDSLSRYIFEKPRIVGGKAASFTIPANSDAFLPCEAVGNPLPTIHWTRVPSGM |
| NOV29ab | ------------------------------------------------------------ |
| NOV29ac | ------------------------------------------------------------ |
| NOV29ad | ------------------------------------------------------------ |
| NOV29ae | ---------------------------------NSDAFLPCEAVGNPLPTIHWTRVPS-- |
| NOV29af | ---------------------------------NSDAFLPCEAVGNPLPTIHWTRVPS-- |
| NOV29ag | ------------------------------------------------------------ |
| NOV29a | SGLDLSKRKQNSRVQVLPNGTLSIQRVEIQDRGQYLCSASNLFGTDHLHVTLSVVSYPPR |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | -GLDLSKRKQNSRVQVLPNGTLSIQRVEIQDRGQYLCSASNLFGTDHLHVTLSVVSYPPR |
| NOV29y | -GLDLSKRKQNSRVQVLPNGTLSIQRVEIQDRGQYLCSASNLFGTDHLHVTLSVVSYPPR |
| NOV29z | SGLDLSKRKQNSRVQVLPNGTLSIQRVEIQDRGQYLCSASNLFGTDHLHVTLSVVSYPPR |
| NOV29aa | SGLDLSKRKQNSRVQVLPNGTLSIQRVEIQDRGQYLCSASNLFGTDHLHVTLSVVSYPPR |
| NOV29ab | ------------------------------------------------------------ |
| NOV29ac | ------------------------------------------------------------ |
| NOV29ad | ------------------------------------------------------------ |
| NOV29ae | -GLDLSKRKQNSRVQVLPNGTLSIQRVEIQDRGQYLCSASNLFGTDHLHVTLSVVSYPPR |
| NOV29af | -GLDLSKRKQNSRVQVLPNGTLSIQRVEIQDRGQYLCSASNLFGTDHLHVTLSVVSYPPR |
| NOV29ag | ------------------------------------------------------------ |
| NOV29a | ILERRTKEITVHSGSTVELKCRAEGRPSPTVTWILANQTVVSESSQGSRQAVVTVDGTLV |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | ILERRTKEITVHSGSTVELKCRAEGRPSPTVTWILANQTVVSESSQGSRQAVVTVDGTLV |
| NOV29y | ILERRTKEITVHSGSTVELKCRAEGRPSPTVTWILANQTVVSESSQGSRQAVVTVDGTLV |
| NOV29z | ILERRTKEITVHSGSTVELKCRAEGRPSPTVTWILANQTVVSESSQGSRQAVVTVDGTLV |
| NOV29aa | ILERRTKEITVHSGSTVELKCRAEGRPSPTVTWILANQTVVSESSQGSRQAVVTVDGTLV |
| NOV29ab | ------------------------------------------------------------ |
| NOV29ac | ------------------------------------------------------------ |
| NOV29ad | ------------------------------------------------------------ |
| NOV29ae | ILERRTKEITVHSGSTVELKCRAEGRPSPTVTWILANQTVVSESSQGSRQAVVTVDGTLV |
| NOV29af | ILERRTKEITVHSGSTVELKCRAEGRPSPTVTWILANQTVVSESSQGSRQAVVTVDGTLV |
| NOV29ag | ------------------------------------------------------------ |
| NOV29a | LHNLSIYDRGFYKCVASNPGGQDSLLVKIQVIAAPPVILEQRRQVIVGTWGESLKLPCTA |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | LHNLSIYDRGFYKCVASNPGGQDSLLVKIQVIAAPPVILEQRRQVIVGTWGESLKLPCTA |
| NOV29y | LHNLSIYDRGFYKCVASNPGGQDSLLVKIQVIAAPPVILEQRRQVIVGTWGESLKLPCTA |
| NOV29z | LHNLSIYDRGFYKCVASNPGGQDSLLVKIQVIAAPPVILEQRRQVIVGTWGESLKLPCTA |
| NOV29aa | LHNLSIYDRGFYKCVASNPGGQDSLLVKIQVIAAPPVILEQPRQVIVGTWGESLKLPCTA |
| NOV29ab | ------------------------------------------------------------ |
| NOV29ac | ------------------------------------------------------------ |
| NOV29ad | ------------------------------------------------------------ |
| NOV29ae | LHNLSIYDRGFYKCVASNPGGQDSLLVKIQVIAAPPVILEQRRQVIVGTWGESLKLPCTA |
| NOV29af | LHNLSIYDRGFYKCVASNPGGQDSLLVKIQVIAAPPVILEQRRQVIVGTWGESLKLPCTA |
| NOV29ag | ------------------------------------------------------------ |
| NOV29a | KGTPQPSVYWVLSDGTEVKPLQFTNSKLFLFSNGTLYIRNLASSDRGTYECIATSSTGSE |
| NOV29b | ------------------------------------------------------------ |
| NOV29c | ------------------------------------------------------------ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | ------------------------------------------------------------ |
| NOV29i | ------------------------------------------------------------ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | ------------------------------------------------------------ |
| NOV29x | KGTPQPSVYWVLSDGTEVKFLQFTNSKLFLFSNGTLYIRNLASSDRGTYECIATSSTGSE |
| NOV29y | KGTPQPSVYWVLSDGTEVKPLQFTNSKLFLFSNGTLYIRNLASSDRGTYECIATSSTGSE |
| NOV29z | KGTPQPSVYWVLSDGTEVKPLQFTNSKLFLFSNGTLYIRNLASSDRGTYECIATSSTGSE |
| NOV29aa | KGTPQPSVYWVLSDGTEVKPLQFTNSKLFLFSNGTLYIRNLASSDRGTYECIATSSTGSE |
| NOV29ab | ------------------------------------------------------------ |
| NOV29ac | ------------------------------------------------------------ |
| NOV29ad | ------------------------------------------------------------ |
| NOV29ae | KGTPQPSVYWVLSDGTEVKPLQFTNSKLFLFSNGTLYIRNLASSDRGTYECIATSSTGSE |
| NOV29af | KGTPQPSVYWVLSDGTEVKPLQFTNSKLFLFSNGTLYIRNLASSDRGTYECIATSSTGSE |
| NOV29ag | ------------------------------------------------------------ |
| NOV29a | RRVVMLTMEERVTSPRIEAASQKRTEVNFGDKLLLNCSATGEPKPQIMWRLPSKAVVDQQ |
| NOV29b | ------------------------------------------------------------ |
| NOV29c | -------------------GSAAAPFTLEGDKLLLNCSATGEPKPQIMWRLPSKAVVDQQ |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | -------------------GSAAAPFTLEGDKLLLNCSATGEPKPQIMWRLPSKAVVDQQ |
| NOV29i | -------------------GSAAAPFTLEGDKLLLNCSATGEPKPQIMWRLPSKAVVDQQ |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | -------------------GSAAAPFTLEGDKLLLNCSATGEPKPQIMWRLPSKAVVDQQ |
| NOV29x | RRVVMLTMEERVTSPRIEAASQKRTEVNFGDKLLLNCSATGEPKPQIMWRLPSKAVVDQQ |
| NOV29y | RRVVMLTMEERVTSPRIEAASQKRTEVNFGDKLLLNCSATGEPKPQTMWRLPSKAVVDQQ |
| NOV29z | RRVVMLTMEERVTSPRIEAASQKRTEVNFGDKLLLNCSATGEPKPQTMWRLPSKAVVDQQ |
| NOV29aa | RRVVMLTMEERVTSPRIEAASQKRTEVNFGDKLLLNCSATGEPKPQTMWRLPSKAVVDQQ |
| NOV29ab | ------------------------------------------------------------ |
| NOV29ac | -----------------------------GDKLLLNCSATGEPKPQIMWRLPSKAVVDQQ |
| NOV29ad | -----------------------------GDKLLLNCSATGEPKPQIMWRLPSKAVVDQQ |
| NOV29ae | RRVVMLTMEERVTSPRIEAASQKRTEVNFGDKLLLNCSATGEPKPQIMWRLPSKAVVDQQ |
| NOV29af | RRVVMLTMEERVTSPRIEAASQKRTEVNFGDKLLLNCSATGEPKPQIMWRLPSKAVVDQQ |
| NOV29ag | ------------------------------------------------------------ |
| NOV29a | HRVGSWIHVYPNGSLFIGSVTEKDSGVYLCVARNKMGDDLILMHVSLRLKPAKIDHKQYF |
| NOV29b | ------------------------------------------------------------ |
| NOV29c | HRVGSWIHVYPNGSLFIGSVTEKDSGVYLCVARNKMGDDLILMHVSLRLKPAKIDHKQYF |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | HRVGSWIHVYPNGSLFIGSVTEKDSGVYLCVARNKMGDDLILMHVSLRLKPAKIDHKQYF |
| NOV29i | HRVGSWIHVYPNGSLFIGSVTEKDSGVYLCVARNKMGDDLILMHVSLRLKPAKIDHKQYF |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | HRVGSWIHVYPNGSLFIGSVTEKDSGVYLCVARNKMGDDLILMHVSLRLKPAKIDHKQYF |
| NOV29x | HRVGSWIHVYPNGSLFIGSVTEKDSGVYLCVARNKMGDDLILMHVSLRLKPAKIDHKQYF |
| NOV29y | HRVGSWIHVYPNGSLFIGSVTEKDSGVYLCVARNKMGDDLILMHVSLRLKPAKIDHKQYF |
| NOV29z | HRVGSWIHVYPNGSLFIGSVTEKDSGVYLCVARNKMGDDLILMHVSLRLKPAKIDHKQYF |
| NOV29aa | HRVGSWIHVYPNGSLFIGSVTEKDSGVYLCVARNKMGDDLILMHVSLELKPAKIDHKQYF |
| NOV29ab | ------------------------------------------------------------ |
| NOV29ac | HRVGSWIHVYPNGSLFIGSVTEKDSGVYLCVARNKMGDDLILMHVSLRLKPAKIDHKQYF |
| NOV29ad | HRVGSWIHVYPNGSLFIGSVTEKDSGVYLCVARNKMGDDLILMHVSLRLKPAKIDHKQYF |
| NOV29ae | HRVGSWIHVYPNGSLFIGSVTEKDSGVYLCVARNKMGDDLILMHVSLRLKPAKIDHKQYF |
| NOV29af | HRVGSWIHVYPNGSLFIGSVTEKDSGVYLCVARNKMGDDLILMHVSLRLKPAKIDHKQYF |
| NOV29ag | ------------------------------------------------------------ |
| NOV29a | RKQVLHGKDFQVDCKASGSPVPEISWSLPDGTMINNAMQADDSGHRTRRYTLFNNGTLYF |
| NOV29b | ------------------------------------------------------------ |
| NOV29c | RKQVLHGKDFQVDCKASGSPVPEISWSLPDGTMINNAMQADDSGHRTRRYTLFNNGTLYF |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | RKQVLHGKDFQVDCKASGSPVPEISWSLPDGTMINNAMQADDSGHRTRRYTLFNNGTLYF |
| NOV29i | RKQVLHGKDFQVDCKASGSPVPEISWSLPDGTMINNAMQADDSGHRTRRYTLFNNGTLYF |
| NOV29j | ------------------------------------------------------------ |
| NOV29k | ------------------------------------------------------------ |
| NOV29l | ------------------------------------------------------------ |
| NOV29m | ------------------------------------------------------------ |
| NOV29n | ------------------------------------------------------------ |
| NOV29o | ------------------------------------------------------------ |
| NOV29p | ------------------------------------------------------------ |
| NOV29q | ------------------------------------------------------------ |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | RKQVLHGKDFQVDCKASGSPVPEISWSLPDGTMINNAMQADDSGHRTRRYTLFNNGTLYF |
| NOV29x | RKQVLHGKDFQVDCKASGSPVPEISWSLPDGTMINNAMQADDSGHRTRRYTLFNNGTLYF |
| NOV29y | RKQVLHGKDFQVDCKASGSPVPEISWSLPDGTMINNAMQADDSGHRTRRYTLFNNGTLYF |
| NOV29z | RKQVLHGKDFQVDCKASGSPVPEISWSLPDGTMINNAMQADDSGHRTRRYTLFNNGTLYF |
| NOV29aa | RKQVLHGKDFQVDCKASGSPVPEISWSLPDGTMINNAMQADDSGHRTRRYTLFNNGTLYF |
| NOV29ab | ------------------------------------------------------------ |
| NOV29ac | RKQVLHGKDFQVDCKASGSPVPEISWSLPDGTMINNAMQADDSGHRTRRYTLFNNGTLYF |
| NOV29ad | RKQVLHGKDFQVDCKASGSPVPEISWSLPDGTMINNAMQADDSGHRTRRYTLFNNGTLYF |
| NOV29ae | RKQVLHGKDFQVDCKASGSPVPEISWSLPDGTMINNAMQADDSGHRTRRYTLFNNGTLYF |
| NOV29af | RKQVLHGKDFQVDCKASGSPVPEISWSLPDGTMINNAMQADDSGHRTRRYTLFNNGTLYF |
| NOV29ag | ------------------------------------------------------------ |
| NOV29a | NKVGVAEEGDYTCYAQNTLGKDEMKVHLTVITAAPRIRQSNKTNKRIKAGDTAVLDCEVT |
| NOV29b | ------------------------------------------------------------ |
| NOV29c | NKVGVAEEGDYTCYAQNTLGKDEMKVHLTVITAAPRIRQSNKTNKRIKAGDTAVLDCEVT |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | NKVGVAEEGDYTCYAQNTLGKDEMKVHLTVITAAPRIRQSNKTNKRIKAGDTAVLDCEVT |
| NOV29i | NKVGVAEEGDYTCYAQNTLGKDEMKVHLTVITAAPRIRQSNKTNKRIKAGDTAVLDCEVT |
| NOV29j | ----------------------------------------------TLEGDKLLLNCSAT |
| NOV29k | ----------------------------------------------TLEGDKLLLNCSAT |
| NOV29l | ----------------------------------------------TLEGDKLLLNCSAT |
| NOV29m | ----------------------------------------------TLEGDKLLLNCSAT |
| NOV29n | ----------------------------------------------TLEGDKLLLNCSAT |
| NOV29o | ----------------------------------------------TLEGDKLLLNCSAT |
| NOV29p | ----------------------------------------------TLEGDKLLLNCSAT |
| NOV29q | ----------------------------------------------TLEGDKLLLNCSAT |
| NOV29r | -------------------------------------------------------APRPP |
| NOV29s | ----------------------------------------------TLEMKVKGRGITCL |
| NOV29t | ----------------------------------------------TLEMKVKGRGITCL |
| NOV29u | ----------------------------------------------TLEMKVKGRGITCL |
| NOV29v | ----------------------------------------------TLEMKVKGRGITCL |
| NOV29w | NKVGVAEEGDYTCYAQNTLGKDEMKVHLTVITAAPRIRQSNKTNKRIKAGDTAVLDCEVT |
| NOV29x | NKVGVAEEGDYTCYAQNTLGKDEMKVHLTVITAAPRIRQSNKTNKRIKAGDTAVLDCEVT |
| NOV29y | NKVGVAEEGDYTCYAQNTLGKDEMKVHLTVITAAPRIRQSNKTNKRIKAGDTAVLDCEVT |
| NOV29z | NKVGVAEEGDYTCYAQNTLGKDEMKVHLTVITAAPRIRQSNKTNKRIKAGDTAVLDREVT |
| NOV29aa | NKVGVAEEGDYTCYAQNTLGKDEMKVHLTVITAAPRIRQSNKTNKRIKAGDTAVLDCEVT |
| NOV29ab | ------------------------------------------------------------ |
| NOV29ac | NKVGVAEEGDYTCYAQNTLGKDEMKVHLTVITAAPRIRQSNKTNKRIKAGDTAVLDCEVT |
| NOV29ad | NKVGVAEEGDYTCYAQNTLGKDEMKVHLTVITAAPRIRQSNKTNKRIKAGDTAVLDCEVT |
| NOV29ae | NKVGVAEEGDYTCYAQNTLGKDEMKVHLTVITAAPRIRQSNKTNKRIKAGDTAVLDCEVT |
| NOV29af | NKVGVAEEGDYTCYAQNTLGKDEMKVHLTVITAAPRIRQSNKTNKRIKAGDTAVLDCEVT |
| NOV29ag | ------------------------------------------------------------ |
| NOV29a | GDPKPKIFWLLPSNDMISFSIDRYTFHANGSLTINKVKLLDSGEYVCVARNPSGDDTKMY |
| NOV29b | -------------------------------------------TLENVERINLGYNSLVR |
| NOV29c | GDPKPKIFWLLPSNDMISFSIDRYTFHANGSLTINKVKLLDSGEYVCVARNPSGDDTKMY |
| NOV29d | --------------KLACPRRCACYMPTEVHCTFRYLTSIPDSIPPNVERINLGYNSLVR |
| NOV29e | --------------KLACPRRCACYMPTEVHCTFRYLTSIPDSIPPNVERINLGYNSLVR |
| NOV29f | --------------KLACPRRCACYMPTEVHCTFRYLTSIPDSIPPNVERINLGYNSLVR |
| NOV29g | --------------KLACPRRCACYMPTEVHCTFRYLTSIPDSIPPNVERINLGYNSLVR |
| NOV29h | GDPKPKIFWLLPSNDMISFSIDRYTFHANGSLTINKVKLLDSGEYVCVARNPSGDDTKMY |
| NOV29i | GDPKPKIFWLLPSNDMISFSIDRYTFHANGSLTINKVKLLDSGEYVCVARNPSGDDTKMY |
| NOV29j | GEPKPQIMWRLPS-KAVVDQQHRYTFHANGSLTINKVKLLDSGEYVCVARNPSGDDTKMY |
| NOV29k | GEPKPQIMWRLPS-KAVVDQQHRYTFHANGSLTINKVKLLDSGEYVCVARNPSGDDTKMY |
| NOV29l | GEPKPQIMWRLPS-KAVVDQQHRYTFHANGSLTINKVKLLDSGEYVCVARNPSGDDTKMY |
| NOV29m | GEPKPQIMWRLPS-KAVVDQQHRYTFHANGSLTINKVKLLDSGEYVCVARNPSGDDTKMY |
| NOV29n | GEPKPQIMWRLPS-KAVVDQQHRYTFHANGSLTINKVKLLDSGEYVCVARNPSGDDTKMY |
| NOV29o | GEPKPQIMWRLPS-KAVVDQQHRYTFHANGSLTINKVKLLDSGEYVCVARNPSGDDTKMY |
| NOV29p | GEPKPQIMWRLQS-KAVVDQQHRYTFHANGSLTINKVKLLDSGEYVCVARNPSGDDTKMY |
| NOV29q | GEPKHQIMWRLPS-KAVVDQQHRYTFHANGSLTINKVKLLDSGEYVCVARNPSGDDTKMY |
| NOV29r | PSPSSVLAAVPVICTLECPRRCACYMPTEVHCTFRYLTSIPDSIPPNVERINLGYNSLVR |
| NOV29s | LVSFAVICLVATPGGKACPRRCACYMPTEVHCTFRYLTSIPDSIPPNVERINLGYNSLVR |
| NOV29t | LVSFAVICLVATPGGKACPRRCACYMPTEVHCTFRYLTSIPDSIPPNVERINLGYNSLVR |
| NOV29u | LVSFAVICLVATPGGKACPRRCACYMPTEVHCTFRYLTSIPDSIPPNVERINLGYNSLVR |
| NOV29v | LVSFAVICLVATPGGKACPRRCACYMPTEVHCTFRYLTSIPDSIPPNVERINLGYNSLVR |
| NOV29w | GDPKPKIFWLLPSNDMISFSIDRYTFHANGSLTINKVKLLDSGEYVCVARNPSGDDTKMY |
| NOV29x | GDPKPKIFWLLPSNDMISFSIDRYTFHANGSLTINKVKLLDSGEYVCVARNPSGDDTKMY |
| NOV29y | GDPKPKIFWLLPSNDMISFSIDRYTFHANGSLTINKVKLLDSGEYVCVARNPSGDDTKMY |
| NOV29z | GDPKPKIFWLLPS---------------NGSLTINKVKLLDSGEYVCVARNPSGDDTKMY |
| NOV29aa | --------------------------HANGSLTINKVKLLDSGEYVCVARNPSGDDTKMY |
| NOV29ab | ----------------ACPRRCACYMPTEVHCTFRYLTSIPDSIPPNVERINLGYNSLVR |
| NOV29ac | GDPKPKIFWLLPSNDMISFSIDRYTFHANGSLTINKVKLLDSGEYVCVARNPSGDDTKIY |
| NOV29ad | GDPKPKIFWLLPSNDMISFSIDRYTFHANGSLTINKVKLLDSGEYVCVARNPSGDDTKIY |
| NOV29ae | GDPKPKIFWLLPSNDMISFSIDRYTFHANGSLTINKVKLLDSGEYVCVARNPSGDDTKIY |
| NOV29af | GDPKPKIFWLLPSNDMISFSIDRYTFHANGSLTINKVKLLDSGEYVCVARNPSGDDTKIY |
| NOV29ag | ----------------ACPRRCACYMPTEVHCTFRYLTSIPDSIPPNVERINLGYNSLVR |
| NOV29a | KLDVVSKPPLINGLYTNRTVIKATAVRHSKKHFDCPAEGTPSPEVMWIMPDNIFLTAPYY |
| NOV29b | LMETDFSGLTKLELLMLHSNGIHTIPDKTFSDLQALQVLKMSYNKVRKLQKDTFYGLRSL |
| NOV29c | KLDVVSKPPLINGLYTNRTVIKATAVRHSKKHFDCRAEGTPSPEVMWIMPDNIFLTAPYY |
| NOV29d | LMETDFSGLTKLELLMLHSNGIHTIPDKTFSDLQALQVLKMSYNKVRKLQKDTFYGLRSL |
| NOV29e | LMETDFSGLTKLELLMLHSNGIHTIPDKTFSDLQALQVLKMSYNKVRKLQKDTFYGLRSL |
| NOV29f | LMETDFSGLTKLELLMLHSNGIHTIPDKTFSDLQALQVLKMSYNKVRKLQKDTFYGLRSL |
| NOV29g | LMETDFSGLTKLELLMLHSNGIHTIPDKTFSDLQALQVLKMSYNKVRKLQKDTFYGLRSL |
| NOV29h | KLDVVSKPPLINGLYTNRTVIKATAVRHSKKHFDCRAEGTPSPEVMWIMPDNIFLTAPYY |
| NOV29i | KLDVVSKPPLINGLYTNRTVIKATAVRHSKKHFDCRAEGTPSPEVMWIMPDNIFLTAPYY |
| NOV29j | KLDVVSKPPLINGLYTNRTVIKATAVRHSKKHFDCRAEGTPSPEVMWIMPDNIFLTAPYY |
| NOV29k | KLDVVSKPPLINGLYThRTVIKATAVRHSKKHFDCPAEGTPSPEVMWIMPDNIFLTAPYY |
| NOV29l | KLDVVSKPPLINGLYTNRTVIKATAVRHSKKHFDCRAEGTPSPEVMWIMPDNIFLTAPYY |
| NOV29m | KLDVVSKPPLINGLYTNRTVIKATAVRHSKKHFDCRAEGTPSPEVMWIMPDNIFLTAPYY |
| NOV29n | KLDVVSKPPLINGLYTNRTVIKATAVRHSKKHFDCRAEGTPSPEVMWIMPDNIFLTAPYY |
| NOV29o | KLDVVSKPPLINGLYTNRTVIKATAMRHSKKHFDCRAEGTPSPEVMWIMPDNIFLTAPYY |
| NOV29p | KLDVVSKPPLINGLYTNRTVIKATAVRHSKKHFDCRAEGTPSPEVMWIMPDNIFLTAPYY |
| NOV29q | KLDVVSKPPLINGLYTNRTVIKATAVRHSKKHFDCRAEGTPSPEVMWIMPDNIFLTAPYY |
| NOV29r | LMETDFSGLTKLELLMLHSNGIHTIPDKTFSDLQALQVLKMSYNKVRKLQKDTFYGLRSL |
| NOV29s | LMETDFSGLTKLELLMLHSNGIHTIPDKTFSDLQALQVLKMSYNKVRKLQKDTFYGLRSL |
| NOV29t | LMETDFSGLTKLELLMLHSNGIHTIPDKTFSDLQALQVLKMSYNKVRKLQKDTFYGLRSL |
| NOV29u | LMETDFSGLTKLELLMLHSNGIHTIPDKTFSDLQALQVLKMSYNKVRKLQKDTFYGLRSL |
| NOV29v | LMETDFSGLTKLELLMLHSNGIHTIPDKTFSDLQALQVLKMSYNKVRKLQKDTFYGLRSL |
| NOV29w | KLDVVSKPPLINGLYTNRTVIKATAVRHSKKHFDCRAEGTPSPEVMWIMPDNIFLTAPYY |
| NOV29x | KLDVVSKPPLINGLYTNRTVIKATAVRHSKKHFDCRAEGTPSPEVMWIMPDNIFLTAPYY |
| NOV29y | KLDVVSKPPLINGLYTNRTVIKATAVRHSKKHFDCRAEGTPSPEVMWIMPDNIFLTAPYY |
| NOV29z | KLDVVSKPPLINGLYTNRTVIKATAVRHSKKHFDCRAEGTPSPEVMWIMPDNIFLTAPYY |
| NOV29aa | KLDVVSKPPLINGLYTNRTVIKATAVRHSKKHFDCRAEGTPSPEVMWIMPDNIFLTAPYY |
| NOV29ab | LMETDFSGLTKLELLMLHSNGIHTIPDKTFSDLQALQVLKMSYNKVRKLQKDTFYGLRSL |
| NOV29ac | KLDVVSKPPLINGLYTNRTVIKATAVRHSKKHFDCRAEGTPSPEVMWIMPDNIFLTAPYY |
| NOV29ad | KLDVVSKPPLINGLYThRTVIKATAVRHSKKHFDCRAEGTPSPEVMWIMPDNIFLTAPYY |
| NOV29ae | KLDVVSKPPLINGLYTNRTVIKATAVRHSKKHFDCRAEGTPSPEVMWIMPDNIFLTAPYY |
| NOV29af | KLDVVSKPPLINGLYTNRTVIKATAVRHSKKHFDCPAEGTPSPEVMWIMPDNIFLTAPYY |
| NOV29ag | LMETDFSGLTKLELLMLHSNGIHTIPDKTFSDLQALQVLKMSYNKVRKLQKDTFYGLRSL |
| NOV29a | GSRITVHKNGTLEIRNVRLSDSADFICVARNEGGESVLVVQLEVLEMLRRPTFRNPFNEK |
| NOV29b | TRLHMDHNN--IEFINPEVFDGLNFLRLVHLEGNQLTKLHPDTFVSLSYLQIFKISFIKF |
| NOV29c | GSRITVHKNGTLEIRNVRLSDSADFICVARNEGGESVLVVQLEVLEMLRRPTFRNPFNEK |
| NOV29d | TRLHMDHNN--IEFINPEVFYGLNFLRLVHLEGNQLTKLHPDTFVSLSYLQIFKISFIKF |
| NOV29e | TRLHMDHNN--IEFINPEVFYGLNFLRLVHLEGNQLTKLHPDTFVSLSYLQIFKISFIKF |
| NOV29f | TRLHMDHNN--IEFINPEVFDGLNFLRLVHLEGNQLTKLHPDTFVSLSYLQIFKISFIKF |
| NOV29g | TRLHMDHNN--IEFINPEVFDGLNFLRLVHLEGNQLTKLHPDTFVSLSYLQIFKISFIKF |
| NOV29h | GSRITVHKNGTLEIRNRLSDSADFICVARNEGGESVLVVQLEVLEMLRRPTFRNPFNEK |
| NOV29i | GSRITVHKNGTLEIRNRLSDSADFICVARNEGGESVLVVQLEVLEMLRRPTFRNPFNEK |
| NOV29j | GSRITVHKNGTLEIRNVRLSDSADFICVARNEGGESVLVVQLEVLEMLRRPTFRNPFNEK |
| NOV29k | GSRITVHKNGTLEIRNVRLSDSADFICVARNEGGESVLVVQLEVLEMLRRPTFRNPFNEK |
| NOV29l | GSRITVHKNGTLEIRNVRLSDSADFICVARNEGGESVLVVQLEVLEMLRRPTFRNPFNEK |
| NOV29m | GSRITVHKNGTLEIRNVRLSDSADFICVARNEGGESVLVVQLEVLEMLRRPTFRNPFNEK |
| NOV29n | GSRITVHKNGTLEIRNVRLSDSADFICVARNEGGESVLVVQLEVLEMLRRPTFRNPFNEK |
| NOV29o | GSRITVHKNGTLEIRNVRLSDSADFICVARNEGGESVLVVQLEVLEMLRRPTFRNPFNEK |
| NOV29p | GSRITVHKNGTLEIRNVRLSDSADFICVARNEGGESVLVVQLEVLEMLRRPAFRNPFNEK |
| NOV29q | GSRITVHKNGTLEIRNVRLSDSADFICVARNEGGESVLVVQLEVLEMLRRPTFRNPFNEK |
| NOV29r | TRLHMDHNN--IEFINPEVFDGLNFLRLVHLEGNQLTKLHPDTFVSLSYLQIFKISFIKF |
| NOV29s | TRLHMDHNN--IEFINPEVFYGLNFLRLVHLEGNQLTKLHPDTFVSLSYLQIFKISFIKF |
| NOV29t | TRLHMDHNN--IEFINPEVFYGLNFLRLVHLEGNQLTKLHPDTFVSLSYLQIFKISFIKF |
| NOV29u | TRLHMDHNN--IEFINPEVFYGLNFLRLVHLEGNQLTKLHPDTFVSLSYLQIFKISFIKF |
| NOV29v | TRLHMDHNN--IEFINPEVFYGLNFLRLVHLEGNQLTKLHPDTFVSLSYLQIFKISFIKF |
| NOV29w | GSRITVHKNGTLEIRYVRLSDSADFICVARNEGGESVLVVQLEVLEMLRRPTFRNPFNEK |
| NOV29x | GSRITVHKNGTLEIRNVRLSDSADFICVARNEGGESVLVVQLEVLEMLRRPTFRNPFNEK |
| NOV29y | GSRITVHKNGTLEIRNVRLSDSADFICVARNEGGESVLVVQLEVLEMLRRPTFRNPFNEK |
| NOV29z | GGRITVHKNGTLEIRNVRLSDSADFICVARNEGGESVLVVQLEVLEMLRRPTFRNPFNEK |
| NOV29aa | GSRITVHKNGTLEIRNVRLSDSADFICVARNEGGESVLVVQLEVLEMLRRPTFRNPFNEK |
| NOV29ab | TRLHMDHNN--IEFINPEVFDGLNFLRLVHLEGNQLTKLHPDTFVSLSYLQIFKISFIKF |
| NOV29ac | GSRITVHKNGTLEIRNVRLSDSADFICVARNEGGESVLVVQLEVLEMLRRPTFRNPFNEK |
| NOV29ad | GSRITVHKNGTLEIRNVRLSDSADFICVARNEGGESVLVVQLEVLEMLRRPTFRNPFNEK |
| NOV29ae | GSRITVHKNGTLEIRNVRLSDSADFICVARNEGGESVLVVQLEVLEMLRRPTFRNPFNEK |
| NOV29af | GSRITVHKNGTLEIRNVRLSDSADFICVARNEGGESVLVVQLEVLEMLRRPTFRNPFNEK |
| NOV29ag | TRLHMDHNN--IEFINPEVFDGLNFLRLVHLEGNQLTKLHPDTFVSLSYLQIFKISFIKF |
| NOV29a | IVAQLGKSTALNCSVDGNPPPEIIWILPNGTRFSNGPQSYQYLIASNGSFIISKTTREDA |
| NOV29b | LYLSDNFLTSLPQEMVSYMP------LEG------------------------------- |
| NOV29c | IVAQLGKSTALNCSVDGNPPPEIIWILPNGTRFSNGPQSYQYLIASNGSFIISKTTREDA |
| NOV29d | LYLSDNFLTSLPQEMVSYMPDLDSLYLHGNPWTCDCHLKWLSDWIQEKPDVIKCKKDRSP |
| NOV29e | LYLSDNFLTSLPQEMVSYMPDLDSLYLHGNPWTCDCHLKWLSDWIQEKPDVIKCKKDRSP |
| NOV29f | LYLSDNFLTSLPQEMVSYMPDLDSLYLHGNPWTCDCHLKWLSDWIQEKPDVIKCKKDRSP |
| NOV29g | LYLSDNFLTSLPQEMVSYMPDLDSLYLHGNPWTCDCHLKWLFDWIQEKPDVIKCKKDRSP |
| NOV29h | IVAQLGKSTALNCSVDGNPPPEIIWILPNGTRFSNGPQSYQYLIASNGSFIISKTTREDA |
| NOV29i | IVAQLGKSTALNCSVDGNPPPEIIWILPNGTRFSNGPQSYQYLIASNGSFIISKTTREDA |
| NOV29j | IVAQLGKSTALNCSVDGNPPPEIIWILPNGTRFSNGPQSYQYLIASNGSFIISKTTREDA |
| NOV29k | IVAQLGKSTALNCSVDGNPPPEIIWILPNGTRFSNGPQSYQYLIASNGSFIISKTTREDA |
| NOV29l | IVAQLGKSTALNCSVDGNPPPEIIWILPNGTRFSNGPQSYQYLIASNGSFIISKTTREDA |
| NOV29m | IVAQLGKSTALNCSVDGNPPPEIIWILPNGTRFSNGPQSYQYLIASNGSFIISKTTREDA |
| NOV29n | IVAQLGKSTALNCSVDGNPPPEIIWILPNGTRFSNGPQSYQYLIASNGSFIISKTTREDA |
| NOV29o | IVAQLGKSTALNCSVDGNPPPEIIWILPNGTRFSNGPQSYQYLIASNGSFIISKTTREDA |
| NOV29p | IVAQLGKSTALNCSVDGNPPPEIIWILPNGTRFSNGPQSYQYLIASNGSFIISKTTREDA |
| NOV29q | IVAQLGKSTALNCSVDGNPPPEIIWILPNGTRFSNGPQSYQYLIASNGSFIISKTTREDA |
| NOV29r | LYLSDNFLTSLPQEMVSYMP------LEGKGGRA-------------------------- |
| NOV29s | LYLSDNFLTSLPQEMVSYMP------LEG------------------------------- |
| NOV29t | LYLSDNFLTSLPQEMVSYMP------LEG------------------------------- |
| NOV29u | LYLSDNFLTSLPQEMVSYMP------LEG------------------------------- |
| NOV29v | LYLSDNFLTSLPQEMVSYMP------LEG------------------------------- |
| NOV29w | IVAQLGKSTALNCSVDGNPPPEIIWILPNGTRFSNGPQSYQYLIASNGSFIISKTTREDA |
| NOV29x | IVAQLGKSTALNCSVDGNPPPEIIWILPNGTRFSNGPQSYQYLIASNGSFIISKTTREDA |
| NOV29y | IVAQLGKSTALNCSVDGNPPPEIIWILPNGTRFSNGPQSYQYLIASNGSFIISKTTREDA |
| NOV29z | IVAQLGKSTALNCSVDGNPPPEIIWILPNGTRFSNGPQSYQYLIASNGSFIISKTTREDA |
| NOV29aa | IVAQLGKSTALNCSVDGNPPPEIIWILPNGTRFSNGPQSYQYLIASNGSFIISKTTREDA |
| NOV29ab | LYLSDNFLTSLPQEMVSYMPDLDSLYLHGNPWTCDCHLKWLSDWIQEKPDVIKCKKDRSP |
| NOV29ac | IVAQLGKSTALNCSVDGNPPPEIIWILPNGTRFSNGPQSYQYLIASNGSFIISKTTREDA |
| NOV29ad | IVAQLGKSTALNCSVDGNPPPEIIWILPNGTRFSNGPQSYQYLIASNGSFIISKTTREDA |
| NOV29ae | IVAQLGKSTALNCSVDGNPPPEIIWILPNGTRFSNGPQSYQYLIASNGSFIISKTTREDA |
| NOV29af | IVAQLGKSTALNCSVDGNPPPEIIWILPNGTRFSNGPQSYQYLIASNGSFIISKTTREDA |
| NOV29ag | LYLSDNFLTSLPQEMVSYMPDLDSLYLHGNPWTCDCHLKWLSDWIQEKPDVIKCKKDRSP |
| NOV29a | GKYRCAARNKVGYIEKLVILEIGQKPVILTYAPGTVKGISGESLSLHCVSDGIPKPNIKW |
| NOV29b | ------------------------------------------------------------ |
| NOV29c | GKYRCAARNKVGYIEKLVILEIGQKPVILTYAPGTVKGISGESLSLHCVSDGIPKPNIKW |
| NOV29d | SSAQQCPLCMNPRTSKGKPLAMVSAAAFQCALE--------------------------- |
| NOV29e | SSAQQCPLCMNPRTSKGKPLAMVSAAAFQCALE--------------------------- |
| NOV29f | SSAPQCPLCNNPRTSKGKPLAMVSAAAFQCALE--------------------------- |
| NOV29g | SSAQQCPLCMNPRTSKGKPLAMVSAAAFQCALE--------------------------- |
| NOV29h | GKYRCAARNKVGYIEKLVILEIGQKPVILTYAPGTVKGISGESLSLHCVSDGIPKPNIKW |
| NOV29i | GKYRCAARNKVGYIEKLVILEIGQKPVILTYAPGTVKGISGESLSLHCVSDGIPKPNIKW |
| NOV29j | GKNRCAARNKVGYIEKLVILEIGQKPVILTYAPGTVKGISGESLSLHCVSDGIPKPNIKW |
| NOV29k | GKYRCAARNKVGYIEKLVILEIGQKPVILTYAPGTVKGISGESLSLHCVSDGIPKPNIKW |
| NOV29l | GKYRCAARNKVGYIEKLVILEIGQKPVILTYAPGTVKGISGESLSLHCVSDGIPKPNIKW |
| NOV29m | GKYRCAARNKVGYIEKLVILEIGQKPVILTYAPGTVKGISGESLSLHCVSDGIPKPNIKW |
| NOV29n | GKYRCAARNKVGYIEKLVILEIGQKPVILTYAPGTVKGISGESLSLHCVSDGIPKPNIKW |
| NOV29o | GKYRCAARNKVGYIEKLVILEIGQKPVILTYAPGTVKGISGESLSLHCVSDGIPKPNIKW |
| NOV29p | GKYRCAAPNKVGYIEKLVILEIGQKPVILTYAPGTVKGISGESLSLHCVSDGIPKPNIKW |
| NOV29q | GKYRCAARNKVGYIEKLVILEIGQKPVILTYAPGTVKGISGESLSLHCVSDGIPKPNIKW |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | GKYRCAARNKVGYIEKLVILEIGQKPVILTYAPGTVKGISGESLSLHCVSDGIPKPNIKW |
| NOV29x | GKYRCAARNKVGYIEKLVILEIGQKPVILTYAPGTVKGISGESLSLHCVSDGIPKPNIKW |
| NOV29y | GKYRCAARNKVGYIEKLVILEIGQKPVILTYAPGTVKGISGESLSLHCVSDGIPKPNIKW |
| NOV29z | GKYRCAARNKVGYIEKLVILEIGQKPVILTYAPGTVKGISGESLSLHCVSDGIPKPNIKW |
| NOV29aa | GKYRCAARNKVGYIEKLVILEIGQKPVILTYAPGTVKGISGESLSLHCVSDGIPKPNIKW |
| NOV29ab | SSAQQCPLCMNPRTSKGKPLAMVSAAAFQCA----------------------------- |
| NOV29ac | GKYRCAARNKVGYIEKLVILEIGQKPVILTYAPGTVKGISGESLSLHCVSDGIPKPNIKW |
| NOV29ad | GKYRCAARNKVGYIEKLVILEIGQKPVILTYAPGTVKGISGESLSLHCVSDGIPKPNIKW |
| NOV29ae | GKYRCAARNKVGYIEKLVILEIGQKPVILTYAPGTVKGISGESLSLHCVSDGIPKPNIKW |
| NOV29af | GKYRCAARNKVGYIEKLVILEIGQKPVILTYAPGTVKGISGESLSLHCVSDGIPKPNIKW |
| NOV29ag | SSAQQCPLCMNPRTSKGKPLAMVSAAAFQCA----------------------------- |
| NOV29a | TMPSGYVVDRPQINGKYILHDNGTLVIKEATAYDRGNYICKAQNSVGHTLITVPVMIVAY |
| NOV29b | ------------------------------------------------------------ |
| NOV29c | TMPSGYVVDRPQINGKYILHDNGTLVIKEATAYDRGNYICKAQNSVGHTLITVPVMIVAY |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | TMPSGYVVDRPQINGKYILHDNGTLVIKEATAYDRGNYICKAQNSVGHTLITVPVMIVAY |
| NOV29i | TMPSGYVVDRPQINGKYILHDNGTLVIKEATAYDRGNYICKAQNSVGHTLITVPVMIVAY |
| NOV29j | TMPSGYVVDRPQINGKYILHDNGTLVIKEATAYDRGNYICKAQNSVGHTLITVPVMIVAY |
| NOV29k | TMPSGYVVDRPQINGKYILHDNGTLVIKEATAYDRGNYICKAQNSVGHTLITVPVMIVAY |
| NOV29l | TMPSGYVVDRPQINGKYILHDNGTLVIKEATAYDRGNYICKAQNSVGHTLITVPVMIVAY |
| NOV29m | TMPSGYVVDRPQINGKYILHDNGTLVIKEATAYDRGNYICKAQNSVGHTLITVPVMIVAY |
| NOV29n | TMPSGYVVDRPQINGKYILHDNGTLVIKEATAYDRGNYICKAQNSVGHTLITVPVMIVAY |
| NOV29o | TMPSGYVVDRPQINGKYILHDNGTLVIKEATAYDRGNYICKAQNSVGHTLITVPVMIVAY |
| NOV29p | TMPSGYVVDRPQINGKYILHDNGTLVIKEATAYDRGNYICKAQNSVGHTLITVPVMIVAY |
| NOV29q | TMPSGYVVDRPQINGKYILHDNGTLVIKEATAYDRGNYICKAQNSVGHTLITVPVMIVAY |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | TMPSGYVVDRPQINGKYILHDNGTLVIKEATAYDRGNYICKAQNSVGHTLITVPVMIVAY |
| NOV29x | TMPSGYVVDRPQINGKYILHDNGTLVIKEATAYDRGNYICKAQNSVGHTLITVPVMIVAY |
| NOV29y | TMPSGYVVDRPQINGKYILHDNGTLVIKEATAYDRGNYICKAQNSVGHTLITVPVMIVAY |
| NOV29z | TMPSGYVVDRPQINGKYILHDNGTLVIKEATAYDRGNYICKAQNSVGHTLITVPVMIVAY |
| NOV29aa | TMPSGYVVDRPQINGKYILHDNGTLVIKEATAYDRGNYICKAQNSVGHTLITVPVMIVAY |
| NOV29ab | ------------------------------------------------------------ |
| NOV29ac | TMPSGYVVDRPQINGKYILHDNGTLVIKEATAYDRGNYICKAQNSVGHTLITVPVMIVAY |
| NOV29ad | TMPSGYVVDRPQINGKYILHDNGTLVIKEATAYDRGNYICKAQNSVGHTLITVPVMIVAY |
| NOV29ae | TMPSGYVVDRPQINGKYILHDNGTLVIKEATAYDRGNYICKAQNSVGHTLITVPVMIVAY |
| NOV29af | TMPSGYVVDRPQINGKYILHDNGTLVIKEATAYDRGNYICKAQNSVGHTLITVPVMIVAY |
| NOV29ag | ------------------------------------------------------------ |
| NOV29a | PPRITNRPPRSIVTRTGAAFQLHCVALGVPKPEITWEMPDHSLLSTASKERTHGSEQLHL |
| NOV29b | ------------------------------------------------------------ |
| NOV29c | PPRITNRPPRSIVTRTGAAFQLHCVALGVPKPEITWEMPDHSLLSTASKERTHGSEQLHL |
| NOV29d | ------------------------------------------------------------ |
| NOV29e | ------------------------------------------------------------ |
| NOV29f | ------------------------------------------------------------ |
| NOV29g | ------------------------------------------------------------ |
| NOV29h | PPRITNRPPRSIVTRTGAAFQLHCVALGVPKPEITWEMPDHSLLSTASKERTHGSEQLHL |
| NOV29i | PPRITNRPPRSIVTRTGAAFQLHCVALGVPKPEITWEMPDHSLLSTASKERTHGSEQLHL |
| NOV29j | PPRITNRPPRSIVTRTGAAFQLHCVALGVPKPEITWEMPDHSLLSTASKERTHGSEQLHL |
| NOV29k | PPRITNRPPRSIVTRTGAAFQLHCVALGVPKPEITWEMPDHSLLSTASKERTHRSEQLHL |
| NOV29l | PPRITNRPPRSIVTRTGAAFQLHCVALGVPKPEITWEMPDHSLLSTASKERTHGSEQLHL |
| NOV29m | PPRITNRPPRSIVTRTGAAFQLHCVALGVPKPEITWEMPDHSLLSTASKERTHGSEQLHL |
| NOV29n | PPRITNRPPRSIVTRTGAAFQLHCVALGVPKPEITWEMPDHSLLSTASKERTHGSEQLHL |
| NOV29o | PPRITNRPPRSIVTRTGAAFQLHCVALGVPKPEITWEMPDHSLLSTASKERTHGSEQLHL |
| NOV29p | PPRITNRPPRSIVTRTGAAFQLHCVALGVPKPEITWEMPDHSLLSTASKERTHGSEQLHL |
| NOV29q | PPRITNRPPRSIVTRTGAAFQLHCVALGVPKPEITWEMSDHSLLSTASKERTHGSEQLHL |
| NOV29r | ------------------------------------------------------------ |
| NOV29s | ------------------------------------------------------------ |
| NOV29t | ------------------------------------------------------------ |
| NOV29u | ------------------------------------------------------------ |
| NOV29v | ------------------------------------------------------------ |
| NOV29w | PPRITNRPPRSIVTRTGAAFQLHCVALGVPKPEITWEMPDHSLLSTASKERTHGSEQLHL |
| NOV29x | PPRITNRPPRSIVTRTGAAFQLHCVALGVPKPEITWEMPDHSLLSTASKERTHGSEQLHL |
| NOV29y | PPRITNRPPRSIVTRTGAAFQLHCVALGVPKPEITWEMPDHSLLSTASKERTHGSEQLHL |
| NOV29z | PPRITNRPPRSIVTRTGAAFQLHCVALGVPKPEITWEMPDHSLLSTASKERTHGSEQLHL |
| NOV29aa | PPRITNRPPRSIVTRTGAAFQLHCVALGVPKPEITWEMPDHSLLSTASKERTHGSEQLHL |
| NOV29ab | ------------------------------------------------------------ |
| NOV29ac | PPRITNRPPRSIVTRTGAAFQLHCVALGVPKPEITWEMPDHSLLSTASKERTHGSEQLHL |
| NOV29ad | PPRITNRPPRSIVTRTGAAFQLHCVALGVPKPEITWEMPDHSLLSTASKERTHGSEQLHL |
| NOV29ae | PPRITNRPPRSIVTRTGAAFQLHCVALGVPKPEITWEMPDHSLLSTASKERTHGSEQLHL |
| NOV29af | PPRITNRPPRSIVTRTGAAFQLHCVALGVPKPEITWEMPDHSLLSTASKERTHGSEQLHL |
| NOV29ag | ------------------------------------------------------------ |
| NOV29a | QGTLVIQNPQTSDSGIYKCTAKNPLGSDYAATYIQVI-------- |
| NOV29b | --------------------------------------------- |
| NOV29c | QGTLVIQNPQTSDSGIYKCTALE----------------GKGGRA |
| NOV29d | --------------------------------------------- |
| NOV29e | --------------------------------------------- |
| NOV29f | --------------------------------------------- |
| NOV29g | --------------------------------------------- |
| NOV29h | QGTLVIQNPQTSDSGIYKCTAKNPLGSDYAATYIQVILEGKGGRA |
| NOV29i | QGTLVIQNPQTSDSGIYKCTALE----------------GKGGRA |
| NOV29j | QGTLVIQNPQTSDSGIYKCTAKNPLGSDYAATYIQVILEG----- |
| NOV29k | QGTLVIQNPQTSDSGIYKCTALEG--------------------- |
| NOV29l | QGTLVIQNPQTSDSGIYKCTALEGKGE------------------ |
| NOV29m | QGTLVIQNPQTSDSGIYKCTALEG--------------------- |
| NOV29n | QGTLVIQNPQTSDSGIYKCTAKNPLGSDYAATYIQVILEG----- |
| NOV29o | QGTLVIQNPQTSDSGIYKCTAKNPLGSDYAATYIQVILEG----- |
| NOV29p | QGTLVIQNPQTSDSGIYKCTAKNPLGSDYAATYIQVILEG----- |
| NOV29q | QGTLVIQNPQTSDSGIYKCTAKNPLGSDYAATYIQVILEG----- |
| NOV29r | --------------------------------------------- |
| NOV29s | --------------------------------------------- |
| NOV29t | --------------------------------------------- |
| NOV29u | --------------------------------------------- |
| NOV29v | --------------------------------------------- |
| NOV29w | QGTLVIQNPQTSDSGIYKCTALE----------------GKGGRA |
| NOV29x | QGTLVIQNPQTSDSGIYKCTAKNPLGSDYAATYIQVILEG----- |
| NOV29y | QGTLVIQNPQTSDSGIYKCTAKNPLGSDYAATYIQVILEG----- |
| NOV29z | QGTLVIQNPQTSDSGIYKCTAKNPLGSDYAATYIQVI-------- |
| NOV29aa | QGTLVIQNPQTSDSGIYKCTAKNPLGSDYAATYIQVI-------- |
| NOV29ab | --------------------------------------------- |
| NOV29ac | QGTLVIQNPQTSDSGIYKCTA------------------------ |
| NOV29ad | QGTLVIQNPQTSDSGIYKCTA------------------------ |
| NOV29ae | QGTLVIQNPQTSDSGIYKCTAKNPLGSDYAATYIQVI-------- |
| NOV29af | QGTLVIQNPQTSDSGIYKCTAKNPLGSDYAATYIQVI-------- |
| NOV29ag | --------------------------------------------- |
| NOV29a | (SEQ ID NO: 666) |
| NOV29b | (SEQ ID NO: 668) |
| NOV29c | (SEQ ID NO: 670) |
| NOV29d | (SEQ ID NO: 672) |
| NOV29e | (SEQ ID NO: 674) |
| NOV29f | (SEQ ID NO: 676) |
| NOV29g | (SEQ ID NO: 678) |
| NOV29h | (SEQ ID NO: 680) |
| NOV29i | (SEQ ID NO: 682) |
| NOV29j | (SEQ ID NO: 684) |
| NOV29k | (SEQ ID NO: 686) |
| NOV29l | (SEQ ID NO: 688) |
| NOV29m | (SEQ ID NO: 690) |
| NOV29n | (SEQ ID NO: 692) |
| NOV29o | (SEQ ID NO: 694) |
| NOV29p | (SEQ ID NO: 696) |
| NOV29q | (SEQ ID NO: 698) |
| NOV29r | (SEQ ID NO: 700) |
| NOV29s | (SEQ ID NO: 702) |
| NOV29t | (SEQ ID NO: 704) |
| NOV29u | (SEQ ID NO: 706) |
| NOV29v | (SEQ ID NO: 708) |
| NOV29w | (SEQ ID NO: 710) |
| NOV29x | (SEQ ID NO: 712) |
| NOV29y | (SEQ ID NO: 714) |
| NOV29z | (SEQ ID NO: 716) |
| NOV29aa | (SEQ ID NO: 718) |
| NOV29ab | (SEQ ID NO: 720) |
| NOV29ac | (SEQ ID NO: 722) |
| NOV29ad | (SEQ ID NO: 724) |
| NOV29ae | (SEQ ID NO: 726) |
| NOV29af | (SEQ ID NO: 728) |
| NOV29ag | (SEQ ID NO: 730) |

Further analysis of the NOV29a protein yielded the following properties shown in Table 29C.

TABLE 29C
Protein Sequence Properties NOV29a

| | |
|---|---|
| SignalP analysis: | Cleavage site between residues 29 and 30 |
| PSORT II analysis: | |

| PSG: | a new signal peptide prediction method | |
|---|---|---|
| | N-region: | length 6; pos. chg 3; neg. chg 0 |
| | H-region: | length 20; peak value 10.48 |
| | PSG score: | 6.08 |

| | |
|---|---|
| GvH: | von Heijne's method for signal seq. recognition |
| | GvH score (threshold: −2.1): −1.23 |
| | possible cleavage site: between 28 and 29 |

>>> Seems to have a cleavable signal peptide (1 to 28)

| ALOM: | Klein et al's method for TM region allocation | |
|---|---|---|
| | Init position for calculation: 29 | |
| | Tentative number of TMS(s) for the threshold 0.5: 1 | |
| | Number of TMS(s) for threshold 0.5: 0 | |
| | PERIPHERAL | Likelihood = 1.91 (at 1062) |

| | ALOM score: 0.37 (number of TMSs: 0) | | | |
|---|---|---|---|---|
| MTOP: | Prediction of membrane topology (Hartmann et al.) | | | |
| | Center position for calculation: 14 | | | |
| | Charge difference: −2.0 C(2.0)-N(4.0) | | | |
| | N >= C: N-terminal side will be inside | | | |
| MITDISC: | discrimination of mitochondrial targeting seq | | | |
| | R content: | 3 | Hyd Moment(75): | 5.15 |
| | Hyd Moment(95): | 8.32 | G content: | 4 |
| | D/E content: | 1 | S/T content: | 4 |

| | |
|---|---|
| | Score: −2.83 |
| Gavel: | prediction of cleavage sites for mitochondrial preseq |
| | R-2 motif at 56 FRY\|LT |

NUCDISC: discrimination of nuclear localization signals

| | |
|---|---|
| | pat4: RRHR (3) at 1043 |
| | pat7: PVLRRHR (3) at 1040 |
| | pat7: PQKKNRT (4) at 1341 |
| | bipartite: RRIDPQHWAALLEKAKK at 760 |
| | content of basic residues: 11.1% |
| | NLS Score: 0.81 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: found

ILPN at 2366

RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 89 |
| COIL: | Lupas's algorithm to detect coiled-coil regions |
| | total: 0 residues |

Final Results (k = 9/23):

```
73.9%: nuclear
  8.7%: mitochondrial
  8.7%: extracellular, including cell wall
  4.3%: cytoplasmic
  4.3%: vacuolar
```

>> prediction for CG55776-01 is nuc (k = 23)

A search of the NOV29a protein against the Geneseq database, a proprietary database that contains sequences published in patents and patent publication, yielded several homologous proteins shown in Table 29D.

TABLE 29D
Geneseq Results for NOV29a

| | | NOV29a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| ABG31323 | Human OCP protein - Homo sapiens, | 1 . . . 2617 | 2535/2637 (96%) | 0.0 |
| | 2589 aa. [WO200246364-A2, | 1 . . . 2589 | 2541/2637 (96%) | |
| | 13-JUN-2002] | | | |
| AAB47935 | Human OCP - Homo sapiens, | 1 . . . 2617 | 2534/2637 (96%) | 0.0 |
| | 2587 aa. [US2002022026-A1, | 1 . . . 2587 | 2540/2637 (96%) | |
| | 21-FEB-2002] | | | |
| ABG32896 | Human osteoclast protein (OCP) #2 - | 1 . . . 2617 | 2534/2637 (96%) | 0.0 |
| | Homo sapiens, 2587 aa. | 1 . . . 2587 | 2540/2637 (96%) | |
| | [US2002086825-A1, 04-JUL-2002] | | | |
| ABG32891 | Human osteoclast protein (OCP) #1 - | 1 . . . 2616 | 2533/2636 (96%) | 0.0 |
| | Homo sapiens, 2586 aa. | 1 . . . 2586 | 2539/2636 (96%) | |
| | [US2002086825-A1, 04-JUL-2002] | | | |
| ABG31317 | Human 5 + 3 corrected OCP protein - | 1 . . . 2616 | 2526/2636 (95%) | 0.0 |
| | Homo sapiens, 2586 aa. | 1 . . . 2586 | 2534/2636 (95%) | |
| | [WO200246364-A2, 13-JUN-2002] | | | |

In a BLAST search of public sequence databases, the NOV29a protein was found to have homology to the proteins shown in the BLASTP data in Table 29E.

TABLE 29E
Public BLASTP Results for NOV29a

| | | NOV29a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| Q8NA84 | Hypothetical protein FLJ35750 - Homo | 1982 . . . 2617 | 636/636 | (100%) | 0.0 |
| | sapiens (Human), 650 aa. | 15 . . . 650 | 636/636 | (100%) | |
| AAH31063 | Similar to hypothetical protein | 2016 . . . 2617 | 601/602 | (99%) | 0.0 |
| | 9030224D03 - Homo sapiens (Human), | 1 . . . 602 | 602/602 | (99%) | |
| | 602 aa. | | | | |
| Q9NR99 | Adlican - Homo sapiens (Human), 2828 | 1637 . . . 2616 | 451/980 | (46%) | 0.0 |
| | aa. | 1851 . . . 2827 | 640/980 | (65%) | |
| BAC28314 | Adult male colon cDNA, RIKEN full-length | 2079 . . . 2617 | 419/539 | (77%) | 0.0 |
| | enriched library, clone: 9030224D03 | 1 . . . 539 | 479/539 | (88%) | |
| | product: hypothetical Immunoglobulin and | | | | |
| | major histocompatibility complex | | | | |
| | domain/Immunoglobulin C-2 | | | | |
| | type/Immunoglobulin-like/Immunoglobulin | | | | |
| | subtype containing protein, full insert | | | | |
| | sequence - Mus musculus (Mouse), 539 | | | | |
| | aa (fragment). | | | | |
| Q8N772 | Hypothetical protein FLJ25972 - Homo | 2219 . . . 2617 | 399/399 | (100%) | 0.0 |
| | sapiens (Human), 399 aa. | 1 . . . 399 | 399/399 | (100%) | |

PFam analysis predicts that the NOV29a protein contains the domains shown in the Table 29F.

TABLE 29F
Domain Analysis of NOV29a

| | | Identities/ | |
|---|---|---|---|
| | | Similarities | |
| Pfam | NOV29a | for the | Expect |
| Domain | Match Region | Matched Region | Value |
| LRRNT | 28 . . . 56 | 12/35 (34%) | 0.00015 |
| | | 23/35 (66%) | |
| LRR | 82 . . . 105 | 8/25 (32%) | 0.25 |
| | | 19/25 (76%) | |
| LRR | 134 . . . 157 | 8/25 (32%) | 0.088 |
| | | 21/25 (84%) | |
| LRR | 158 . . . 181 | 12/25 (48%) | 0.32 |
| | | 19/25 (76%) | |
| LRRCT | 223 . . . 291 | 17/69 (25%) | 8e−10 |
| | | 52/69 (75%) | |
| ig | 501 . . . 564 | 16/67 (24%) | 0.59 |
| | | 40/67 (60%) | |
| ig | 599 . . . 658 | 18/63 (29%) | 1.9e−07 |
| | | 44/63 (70%) | |
| ig | 1654 . . . 1719 | 14/69 (20%) | 0.002 |
| | | 46/69 (67%) | |
| ig | 1754 . . . 1816 | 17/66 (26%) | 5.7e−05 |
| | | 45/66 (68%) | |
| ig | 1851 . . . 1913 | 19/66 (29%) | 1e−07 |
| | | 47/66 (71%) | |
| ig | 1950 . . . 2012 | 21/66 (32%) | 5.1e−08 |
| | | 46/66 (70%) | |
| ig | 2047 . . . 2115 | 13/72 (18%) | 0.0002 |
| | | 44/72 (61%) | |
| ig | 2150 . . . 2209 | 20/63 (32%) | 6e−09 |
| | | 45/63 (71%) | |
| ig | 2248 . . . 2309 | 13/65 (20%) | 0.36 |
| | | 38/65 (58%) | |
| ig | 2346 . . . 2407 | 21/65 (32%) | 1.3e−06 |
| | | 44/65 (68%) | |
| ig | 2441 . . . 2502 | 16/65 (25%) | 0.022 |
| | | 41/65 (63%) | |
| ig | 2537 . . . 2601 | 16/68 (24%) | 1.1e−05 |
| | | 46/68 (68%) | |

### Example 30

The NOV30 clone was analyzed, and the nucleotide and encoded polypeptide sequences are shown in Table 30A.

TABLE 30A

NOV30 Sequence Analysis

| | | |
|---|---|---|
| NOV30a, CG55784-03 | SEQ ID NO: 751 | 3772 bp |
| DNA Sequence | ORF Start: ATG at 251 | ORF Stop: TAA at 2549 |

CTTCTCCGAGTGGGGACATTGCTGACAATCCCGGCTTCCCGAGGCGGCTAAGAACAGGCAGTTTGTGT
CGGCTGGCTGCAGATACCCAGAGGCACAAAGAGACCGAAGCCACCCGGAGGGACCCACGGACGGACAG
ATGGTAGGCGCGAACCCGAGAGGACCGGCGGAGGCTGAGCACCGAGAGCCGCCAAGGAAGAGAAACTA
ACCACAGCCAAGTTACCCCGCCGGCTTTCCTTCGCTGCGCTAAGGAATGAAACCCTTCCAGCTCGATC
TGCTCTTCGTCTGCTTCTTCCTCTTCAGTCAAGAGCTGGGCCTCCAGAAGAGAGGATGCTGTCTGGTG
CTGGGCTACATGGCCAAGGACAAGTTTCGGAGAATGAATGAAGGCCAAGTCTATTCCTTCAGCCAGCA
GCCCCAGGACCAGGTGGTGGTGTCGGGACAGCCAGTGACGCTACTTTGCGCCATCCCCGAATACGATG
TACCTGGTGGTAGGGAACCACCTGTCAGGGGAGCACCACCTGAAGATCCTGAGGGCAGAGCTGCAAGA
CGATGCGGTGTACGAGTGCCAGGCCATCCAGGCCGCCATCCGCTCCCGCCCCGCACGCCTCACAGTCC
TGGTGCCGCCTGATGACCCCGTCATCCTGGGGGGCCCTGTGATCAGCCTGCGTGCGGGGGACCCTCTC
AACCTCACCTGCCACGCAGACAATGCCAAGCCTGCAGCCTCCATCATCTGGTTGCGAAAGGGAGAGGT
CATCAATGGGGCCACCTACTCCAAGACCCTGCTTCGGGACGGCAAGCGGGAGAGCATCGTCAGCACCC
TCTTCATCTCCCCTGGTGACGTGGAGAATGGCCAGAGCATCGTGTGTCGTGCCACCAACAAAGCCATC
GCCACAGCCAGTGCTGGAGGACAACGTCGTCACTTTCCACTGCTCTGCAAAGGCCAACCCAGCTGTCA
GTGGACTACACGTACTTCTCAGAGCCCGTCTCCTGTGAGGTGACCAACGCCCTGGGCAGCACCAACCT
CAGCCGCACGGTTGACGTCTACTTTGGGCCCCGGATGACCACAGAACCCCAATCCTTGCTCGTGGATC
TGGGCTCTGATGCCATCTTCAGCTGCGCCTGGACCGGCAACCCATCCCTGACCATCGTCTGGATGAAG
CGGGGCTCCGGAGTGGTCCTGAGCAATGAGAAGACCCTGACCCTCAAATCCGTGCGCCAGGAGGACGC
GGGCAAGTACGTGTGCCGGGCTGTGGTGCCCCGTGTGGGAGCCGGGGAGAGAGAGGTGACCCTGACCG
TCAATGGACCCCCCATCATCTCCAGCACCCAGACCCAGCACGCCCTCCACGGCGAGAAGGGCCAGATC
AAGTGCTTCATCCGGAGCACGCCGCCGCCGGACCGCATCGCCTGGTCCTGGAAGGAGAACGTTCTGGA
GTCGGGCACATCGGGGCGCTATACGGTGGAGACCATCAGCACCGAGGAGGGCGTCATCTCCACCCTGA
CCATCAGCAACATCGTGCGGGCCGACTTCCAGACCATCTACAACTGCACGGCCTGGAACAGCTTCGGC
TCCGACACTGAGATCATCCGGCTCAAGGAGCAAGAGTCTGTGCCGATGGCCGTCATCATTGGGGTGGC
CGTAGGAGCTGGTGTGGCCTTCCTCGTCCTTATGGCAACCATCGTGGCGTTCTGCTGTGCCCGTTCCC
GCCTCTGGTCGGGAGGGTGAGGAGCACTCCACCATCAAGCAGCTGATGATGGACCGGGGTGAATTCCA
GCAAGACTCAGTCCTGAAACAGCTGGAGGTCCTCAAAGAAGAGGAGAAAGAGTTTCAGAACCTGAAGG
ACCCCACCAATGGCTACTACAGCGTCAACACCTTCAAAGAGCACCACTCAACCCCGACCATCTCCCTC
TCCAGCTGCCAGCCCGACCTGCGTCCTGCGGGTAAGCAGCGTGTGCCCACAGGCATGTCCTTCACCAA
CATCTACAGCACCCTGAGCGGCCAGGGCCGCCTCTACGACTACGGGCAGCGGTTTGTGCTGGGCATGG
GCAGCTCGTCCATCGAGCTTTGTGAGCGGGAGTTCCAGAGAGGCTCCCTCAGCGACAGCAGCTCCTTC
CTGGACACGCAGTGTGACAGCAGCGTCAGCAGCAGCGGCAAGCAGGATGGCTATGTGCAGTTCGACAA
GACCCCTGCAGCGGCGGATGCAGACTCACGTCTAAGGATCACACACCGCGGGTGGGGACGGGCCAGGC
AAGAGGTCAGGGCACGTTCTGGTTGTCCAGGGACGAGGGGTACTTTGCAGAGGACACCAGAATTGGCC
ACTTCCAGGACAGCCTCCCAGCGCCTCTGCCACTGCCTTCCTTCGAAGCTCTGATCAAGCACAAATCT
GGGTCCCCAGGTGCTGTGTGCCAGAGGTGGGCGGGTGGGGAGACAGACAGAGGCTGCGGCTGAGTGCG
CTGTGCTTAGTGCTGGACACCCGTGTCCCCGGCCCTTTCCTGGAGGCCCCTCTACCACCTGCTCTGCC
CACAGGCACAAGTGGCAGCTATAACTCTGCTTTCATGAAACTGCGGTCCACTCTCTGGTCTCTCTGTG
GGCTCTACCCCTCACTGACCACAAGCTCTACCTACCCCTGTGCCTGTGCTCCCATACAGCCCTGGGGG
CCATACCCTGGAGGTTGACAAGCCAGAAATGGCCTTGGCTAAAGGAGCCTCTCTCTCACCAGGCTGGC
CGGGAGCCCACCCCCAATTTGTTTGGTGTTTTGTGTCCATACTCTTGCAGTTCTGTCCTTGGACTTGA
TGCCGCTGAACTCTGCGGTGGGACCGGTCCCGTCAGAGCCTGGTGTACTGGGGGGAGGGAGGGAGGAG
GGAGCCTGTGCTGACGGAGCACCTCGCCGGGTGTGCCCCTCCTGGGCTGTGTGACCCCAGCCTCCCCA
CCCACCTCCTGCTTTGTGTACTCCTCCCCTCCCCCTCAGCACAATCGGAGTTCATATAAGAAGTGCGC
GAGCTTCTCTGGTCAGGGTTCTCTGAACACTTATGGAGAGAGTGCTTCCTGGGAAGTGTGGCGTTTGA
AGGGGCTGGAGGGCAGGTCTTTAAGATGGCGAGACTGCCCTTCTCAGCTGATAAACACAAGAACGGCC
ATCCTGTCTTCAGTAAGGCTCCACGAGAAGAGAGGAAGTATATCTACACCTCAACCCTCCTAGTCACC
ACCTGAAATAATGTTAGGGACACTACTCCAACATGTTTGTTCTGTTCTTTTGTTCCTACAAAAGCCAC
AGGAAGAACCCAAGAGCTCATAGAATGCGTTGGGAACCCAAGGTTCTCTGCCCTCCTTTGATTCAATC
ATCCTAGACAATAAAGGCAGTTGATAGCTCTG

| | | | |
|---|---|---|---|
| NOV30a, CG55784-03 | | | |
| Protein Sequence | SEQ ID NO: 752 | 766 aa | MW at 84135.5kD |

MKPFQLDLLFVCFFLFSQELGLQKRGCCLVLGYMAKDKFRRMNEGQVYSFSQQPQDQVVVSGQPVTLL
CAIPEYDGFVLWIKDGLALGVGRDLSSYPQYLVVGNHLSGEHHLKILRAELQDDAVYECQAIQAAIRS
RPARLTVLVPPDDPVILGGPVISLRAGDPLNLTCHADNAKPAASIIWLRKGEVINGATYSKTLLRDGK
RESIVSTLFISPGDVENGQSIVCRATNKAIPGGKETSVTIDIQHPPLVNLSVEPQPVLEDNVVTFHCS
AKANPAVTQYRWAKRGQIIKEASGEVYRTTVDYTYFSEPVSCEVTNALGSTNLSRTVDVYFGPRMTTE
PQSLLVDLGSDAIFSCAWTGNPSLTIVWMKRGSGVVLSNEKTLTLKSVRQEDAGKYVCRAVVPRVGAG
EREVTLTVNGPPIISSTQTQHALHGEKGQIKCFIRSTPPPDRIAWSWKENVLESGTSGRYTVETISTE
EGVISTLTISNIVRADFQTIYNCTAWNSFGSDTEIIRLKEQESVPMAVIIGVAVGAGVAFLVLMATIV
AFCCARSQRNLKGVVSAKNDIRVEIVHKEPASGREGEEHSTIKQLMMDRGEFQQDSVLKQLEVLKEEE
KEFQNLKDPTNGYYSVNTFKEHHSTPTISLSSCQPDLRPAGKQRVPTGMSFTNIYSTLSGQGRLYDYG
QRFVLGMGSSSIELCEREFQRGSLSDSSSFLDTQCDSSVSSSGKQDGYVQFDKASKASASSSHHSQSS
SQNSDPSRPLQRRMQTHV

| | | |
|---|---|---|
| NOV30b, CG55784-01 | SEQ ID NO: 753 | 1806 bp |
| DNA Sequence | ORF Start: at 1 | ORF Stop: TAA at 1645 |

AACAAAGCCATCCCCGGAGGAAAGGAGACGTCGGTCACCATTGACATCCAGCACCCTCCACTGGTCAA
CCTCTCGGTGGAGCCACAGCCAGTGCTGGAGGACAACGTCGTCACTTTCCACTGCTCTGCAAAGGCCA
ACCCAGCTGTCACCCAGTACAGGTGGGCCAAGCGGGGCCAGATCATCAAGGAGGCATCTGGAGAGGTG
TACAGGACCACAGTGGACTACACGTACTTCTCAGAGCCCGTCTCCTGTGAGGTGACCAAAGCCCTGGG
CAGCACCAACCTCAGCCGCACGGTTGACGTCTACTTTGGGCCCCGGATGACCACAGAACCCCAATCCT
TGCTCGTGGATCTGGGCTCTGATGCCATCTTAAGCTGCGCCTGGACCGGAAACCCATCCCTGACCATC
TGACCCTGACCGTCAATGGACCCCCCATCATCTCCAGCACCCAGACCCAGCACGCCCTCCACGGCGAG
AAGGGCCAGATCAAGTGCTTCATCCGGAGCACGCCGCCGCCGGACCGCATCGCCTGGTCCTGGAAGGA
GAACGTTCTGGAGTCGGGCACATCGGGGCGCTATACGGTGGAGACCATCAGCACCGAGGAGGGCGTCA
TCTCCACCCTGACCATCAGCAACATCGTGCGGGCCGACTTCCAGACCATCTACAACTGCACGGCCTGG
AACAGCTTCGGCTCCGACACTGAGATCATCCGGCTCAAGGAGCAAGGTTCGGAAATGAAGTCGGGAGC
CGGGCTGGAAGCAGAGTCTGTGCCGATGGCCGTCATCATTGGGGTGGCCGTAGGAGCTGGTGTGGCCT
TCCTCGTCCTTATGGCAACCATCGTGGCGTTCTGCTGTGCCCGTTCCCAGAGAAATCTCAAAGGTGTT
GTGTCAGCCAAAAATGATATCCGAGTGGAAATTGTCCACAAGGAACCAGCCTCTGGTCGGGAGGGTGA
GGAGCACTCCACCATCAAGCAGCTGATGATGGACCGGGGTGAATTCCAGCAAGACTCAGTCCTGAAAC
AGCTGGAAGGTCCTCAAAGAAGAGGAGAAGAGTTTCAGAACCTGAAGGACCCCACCAATGGCTACTAC
AGCGTCAACACCTTCAAAGAGCACCACTCAACCCCGACCATCTCCCTCTCCAGCTGCCAGCCCGACCT
GCGTCCTGCGGGCAAGCAGCGTGTGCCCACAGGCATGTCCTTCACCAACATCTACAGCACCCTGAGCG
GCCAGGGCCGCCTCTACGACTACGGGCAGCGGTTTGTGCTGGGCATGGGCAGCTCGTCCATCGAGCTT
TGTGAGCGGGAGTTCCAGAGAGGCTCCCTCAGCGACAGCAGCTCCTTCCTGGACACGCAGTGTGACAG
CAGCGTCAGCAGCAGCGGCAAGCAGGATGGCTATGTGCAGTTCGACAAGGCCAGCAAGGCTTCTGCTT
CCTCCTCCCACCACTCCCAGTCCTCGTCCCAGAACTCTGACCCCAGTCGACCCCTGCAGCGGCGGATG
CAGACTCACGTCTAAGGATCACACACCGCGGGTGGGGACGGGCCAGGGAAGAGGTCAGGGCACGTTCT
GGTTGTCCAGGGACTGTGGGGTACTTTACAGAGGACACCAGAATGGCCCACTTCCAGGACAGCCTCCC
AGCGCCTCTGCCACTGCCTTCCTTCGAAGCTCTGATCA

| | | | |
|---|---|---|---|
| NOV30b, CG55784-01 | | | |
| Protein Sequence | SEQ ID NO: 754 | 548 aa | MW at 59933.8kD |

NKAIPGGKETSVTIDIQHPPLVNLSVEPQPVLEDNVVTFHCSAKANPAVTQYRWAKRGQIIKEASGEV
YRTTVDYTYFSEPVSCEVTKALGSTNLSRTVDVYFGPRMTTEPQSLLVDLGSDAILSCAWTGNPSLTI
VWMKRGSGVVLSNEKTLTLKSVRQEDAGKYVCRAVVPRVGAGEREVTLTVNGPPIISSTQTQHALHGE
KGQIKCFIRSTPPPDRIAWSWKENVLESGTSGRYTVETISTEEGVISTLTISNIVPADFQTIYNCTAW
NSFGSDTEIIRLKEQGSEMKSGAGLEAESVPMAVIIGVAVGAGVAFLVLMATIVAFCCARSQRNLKGV
VSAKNDIRVEIVHKEPASGREGEEHSTIKQLMMDRGEFQQDSVLKQLEVLKEEEKEFQNLKDPTNGYY
SVNTFKEHHSTPTISLSSCQPDLRPAGKQRVPTGMSFTNIYSTLSGQGRLYDYGQRFVLGMGSSSIEL
CEREFQRGSLSDSSSFLDTQCDSSVSSSGKQDGYVQFDKASKASASSSHHSQSSSQNSDPSRPLQRRM
QTHV

| | | |
|---|---|---|
| NOV30c, 312000579 | SEQ ID NO: 755 | 1630 bp |
| DNA Sequence | ORF Start: at 2 | ORF Stop: end of sequence |

CACCGGATCCACCAACAAAGCCATCCCCGGAGGAAAGGAGACGTCGGTCACCATTGACATCCAGCACC
CTCCACTGGTCAACCTCTCGGTGGAGCCACAGCCAGTGCTGGAGGACAACGTCGTCACTTTCCACTGC
TCTGCAAAGGCCAACCCAGCTGTCACCCAGTACAGGTGGGCCAAGCGGGGCCAGATCATCAAGGAGGC
ATCTGGAGAGGTGTACAGGACCACAGTGGACTACACGTACTTCTCAGAGCCCGTCTCCTGTGAGGTGA
CCAACGCCCTGGGCAGCACCAACCTCAGCCGCACGGTTGACGTCTACTTTGGGCCCCGGATGACCACA
GAACCCCAATCCTTGCTCGTGGATCTGGGCTCTGATGCCATCTTCAGCTGCGCCTGGACCGGCAACCC
ATCCCTGACCATCGTCTGGATGAAGCGGGGCTCCGGAGTGGTCCTGAGCAATGAGAAGACCCTGACCC
TCAAATCCGTGCGCCAGGAGGACGCGGGCAAGTACGTGTGCCGGGCTGTGGTGCCCCGTGTGGGAGCC
GGGGAGAGAGAGGTGACCCTGACCGTCAATGGACCCCCCATCATCTCCAGCACCCAGACCCAGCACGC
CCTCCACGGCGAGAAGGGCCAGATCAAGTGCTTCATCCGGAGCACGCCGCCGCCGGACCGCATCGCCT
GGTCCTGGAAGGAGAACGTTCTGGAGTCGGGCACATCGGGGCGCTATACGGTGGAGACCATCAGCACC
GAGGAGGGCGTCATCTCCACCCTGACCATCAGCAACATCGTGCGGGCCGACTTCCAGACCATCTACAA
CTGCACGGCCTGGAACAGCTTCGGCTCCGACACTGAGATCATCCGGCTCAAGGAGCAAGAGTCTGTGC
CGATGGCCGTCATCATTGGGGTGGCCGTAGGAGCTGGTGTGGCCTTCCTCGTCCTTATGGCAACCATC
GTGGCGTTCTGCTGTGCCCGTTCCCAGAGAAATCTCAAAGGTGTTGTGTCAGCCAAAAATGATATCCG
AGTGGAAATTGTCCACAAGGAACCAGCCTCTGGTCGGGAGGGTGAGGAGCACTCCACCATCAAGCAGC
TGATGATGGACCGGGGTGAATTCCAGCAAGACTCAGTCCTGAAACAGCTGGAGGTCCTCAAAGAAGAG
GAGAAGAGTTTCAGAACCTGAAGGACCCCACCAATGGCTACTACAGCGTCAACACCTTCAAAAGAGCA
CCACTCAACCCCGACCATCTCCCTCTCCAGCTGCCAGCCCGACCTGCGTCCTGCGGGTAAGCAGCGTG
TGCCCACAGGCATGTCCTTCACCAACATCTACAGCACCCTGAGCGGCCAGGGCCGCCTCTACGACTAC
GGGCAGCGGTTTGTGCTGGGCATGGGCAGCTCGTCCATCGAGCTTTGTGAGCGAAAGTTCCAGAGAGG
CTCCCTCAGCGACAGCAGCTCCTTCCTGGACACGCAGTGTGACAGCAGCGTCAGCAGCAGCGGCAAGC
AGGATGGCTATGTGCAGTTCGACAAGGCCAGCAAGGCTTCTGCTTCCTCCTCCCACCACTCCCAGTCC
TCGTCCCAGAACTCTGACCCCAGTCGACCCCTGCAGCGGCGGATGCAGACTCACGTCCCCGGGGGC

| | | | |
|---|---|---|---|
| NOV30c, 312000579 | | | |
| Protein Sequence | SEQ ID NO: 756 | 543 aa | MW at 59393.1kD |

TGSTNKAIPGGKETSVTIDIQHPPLVNLSVEPQPVLEDNVVTFHCSAKANPAVTQYRWAKRGQIIKEA
SGEVYRTTVDYTYFSEPVSCEVTNALGSTNLSRTVDVYFGPRNTTEPQSLLVDLGSDAIFSCAWTGNP
SLTIVWMKRGSGVVLSNEKTLTLKSVRQEDAGKYVCRAVVPRVGAGEREVTLTVNGPPIISSTQTQHA
LHGEKGQIKCFIRSTPPPDRIAWSWKENVLESGTSGRYTVETISTEEGVISTLTISNIVRADFQTIYN
CTAWNSFGSDTEIIRLKEQESVPMAVIIGVAVGAGVAFLVLMATIVAFCCARSQRNLKGVVSAKNDIR
VEIVHKEPASGREGEEHSTIKQLMMDRGEFQQDSVLKQLEVLKEEEKEFQNLKDPTNGYYSVNTFKEH
HSTPTISLSSCQPDLRPAGKQRVPTGMSFTNTYSTLSGQGRLYDYGQRFVLGMGSSSIELCEREFQRG

| | | |
|---|---|---|
| NOV30d, 311333341 | SEQ ID NO: 757 | 1036 bp |
| DNA Sequence | ORF Start: at 2 | ORF Stop: end of sequence |

CACCGGATCCACCAACAAAGCCATCCCCGGAGGAAAGGAGACGTCGGTCACCATTGACATCCAGCACC
CTCCACTGGTCAACCTCTCGGTGGAGCCACAGCCAGTGCTGGAGGACAACGTCGTCACTTTCCACTGC
TCTGCAAAGGCCAACCCAGCTGTCACCCAGTACAGGTGGGCCAAGCGGGGCCAGATCATCAAGGAGGC
ATCTGGAGAGGTGTACAGGACCACAGTGGACTACACGTACTTCTCAGAGCCCGTCTCCTGTGAGGTGA
CCAACGCCCTGGGCAGCACCAACCTCAGCCGCACGGTTGACGTCTACTTTGGGCCCCGGATGACCACA
GAACCCCAATCCTTGCTCGTGGATCTGGGCTCTGATGCCATCTTCAGCTGCGCCTGGACCGGCAACCC
ATCCCTGACCATCGTCTGGATGAAGCGGGGCTCCGGAGTGGTCCTGAGCAATGAGAAGACCCTGACCC
TCAAATCCGTGCGCCAGGAGGACGCGGGCAAGTACGTGTGCCGGGCTGTGGTGCCCCGTGTGGGAGCC
GGGGAGAGAGAGGTGACCCTGACCGTCAATGGACCCCCCATCATCTCCAGCACCCAGACCCAGCACGC
CCTCCACGGCGAGAAGGGCCAGATCAAGTGCTTCATCCGGAGCACGCCGCCGCCGGACCGCATCGCCT
GGTCCTGGAAGGAGAACGTTCTGGAGTCGGGCACATCGGGGCGCTATACGGTGGAGACCATCAGCACC
GAGGAGGGCGTCATCTCCACCCTGACCATCAGCAACATCGTGCGGGCCGACTTCCAGACCATCTACAA
CTGCACGGCCTGGAACAGCTTCGGCTCCGACACTGAGATCATCCGGCTCAAGGAGCAAGAGTCTGTGC
CGATGGCCGTCATCATTGGGGTGGCCGTAGGAGCTGGTGTGGCCTTCCTCGTCCTTATGGCAACCATC
GTGGCGTTCTGCTGTGCCCGTTCCCAGAGAAATCTCAAAGGTGTTGTGTCAGCCAAAAATGATATCCG
AGTGGAACCCGGGGGC

| | | | |
|---|---|---|---|
| NOV30d, 311333341 | | | |
| Protein Sequence | SEQ ID NO: 758 | 345 aa | MW at 37329.1kD |

TGSTNKAIPGGKETSVTIDIQHPPLVNLSVEPQPVLEDNVVTFHCSAKANPAVTQYRWAKRGQIIKEA
SGEVYRTTVDYTYFSEPVSCEVTNALGSTNLSRTVDVYFGPRMTTEPQSLLVDLGSDAIFSCAWTGNP
SLTIVWMKRGSGVVLSNEKTLTLKSVRQEDAGKYVCRAVVPRVGAGEREVTLTVNGPPIISSTQTQHA
LHGEKGQIKCFIRSTPPPDRIAWSWKENVLESGTSGRYTVETISTEEGVISTLTISNIVRADFQTIYN
CTAWNSFGSDTEIIRLKEQESVPMAVIIGVAVGAGVAFLVLMATIVAFCCARSQRNLKGVVSAKNDIR
VEPGG

| | | |
|---|---|---|
| NOV30e, 311333338 | SEQ ID NO: 759 | 1072 bp |
| DNA Sequence | ORF Start: at 2 | ORF Stop: end of sequence |

CACCGGATCCACCAACAAAGCCATCCCCGGAGGAAAGGAGACGTCGGTCACCATTGACATCCAGCACC
CTCCACTGGTCAACCTCTCGGTGGAGCCACAGCCAGTGCTGGAGGACAACGTCGTCACTTTCCACTGC
TCTGCAAAGGCCAACCCAGCTGTCACCCAGTACAGGTGGGCCAAGCGGGGCCAGATCATCAAGGAGGC
ATCTGGAGAGGTGTACAGGACCACAGTGGACTACACGTACTTCTCAGAGCCCGTCTCCTGTGAGGTGA
CCAACGCCCTGGGCAGCACCAACCTCAGCCGCACGGTTGACGTCTACTTTGGGCCCCGGATGACCACA
GAACCCCAATCCTTGCTCGTGGATCTGGGCTCTGATGCCATCTTCAGCTGCGCCTGGACCGGCAACCC
ATCCCTGACCATCGTCTGGATGAAGCGGGGCTCCGGAGTGGTCCTGAGCAATGAGAAGACCCTGACCC
TCAAATCCGTGCGCCAGGAGGACGCGGGCAAGTACGTGTGCCGGGCTGTGGTGCCCCGTGTGGGAGCC
GGGGAGAGAGAGGTGACCCTGACCGTCAATGGACCCCCCATCATCTCCAGCACCCAGACCCAGCACGC
CCTCCACGGCGAGAAGGGCCAGATCAAGTGCTTCATCCGGAGCACGCCGCCGCCGGACCGCATCGCCT
GGTCCTGGAAGGAGAACGTTCTGGAGTCGGGCACATCGGGGCGCTATACGGTGGAGACCATCAGCACC
GAGGAGGGCGTCATCTCCACCCTGACCATCAGCAACATCGTGCGGGCCGACTTCCAGACCATCTACAA
CTGCACGGCCTGGAACAGCTTCGGCTCCGACACTGAGATCATCCGGCTCAAGGAGCAAGGTTCGGAAA
TGAAGTCGGGAGCCGGGCTGGAAGCAGAGTCTGTGCCGATGGCCGTCATCATTGGGGTGGCCGTAGGA
GCTGGTGTGGCCTTCCTCGTCCTTATGGCAACCATCGTGGCGTTCTGCTGTGCCCGTTCCCAGAGAAA
TCTCAAAGGTGTTGTGTCAGCCAAAAATGATATCCGAGTGGAACCCGGGGGC

| | | | |
|---|---|---|---|
| NOV30e, 311333338 | | | |
| Protein Sequence | SEQ ID NO: 760 | 357 aa | MW at 38447.3kD |

TGSTNKAIPGGKETSVTIDIQHPPLVNLSVEPQPVLEDNVVTFHCSAKANPAVTQYRWAKRGQIIKEA
SGEVYRTTVDYTYFSEPVSCEVTNALGSTNLSRTVDVYFGPRMTTEPQSLLVDLGSDAIFSCAWTGNP
SLTIVWMKRGSGVVLSNEKTLTLKSVRQEDAGKYVCRAVVPRVGAGEREVTLTVNGPPIISSTQTQHA
LHGEKGQIKCFIRSTPPPDRIAWSWKENVLESGTSGRYTVETISTEEGVISTLTISNIVRADFQTIYN
CTAWNSFGSDTEIIRLKEQGSEMKSGAGLEAESVPMAVIIGVAVGAGVAFLVLMATIVAFCCARSQRN
LKGVVSAKNDIRVEPGG

| | | |
|---|---|---|
| NOV30f, 311346885 | SEQ ID NO: 761 | 616 bp |
| DNA Sequence | ORF Start: at 2 | ORF Stop: end of sequence |

CACCGGATCCGAAATTGTCCACAAGGAACCAGCCTCTGGTCGGGAGGGTGAGGAGCACTCCACCATCA
AGCAGCTGATGATGGACCGGGGTGAATTCCAGCAAGACTCAGTCCTGAAACAGCTGGAGGTCCTCAAA
GAAGAGGAGAAAGAGTTTCAGAACCTGAAGGACCCCACCAATGGCTACTACAGCGTCAACACCTTCAA
AGAGCACCACTCAACCCCGACCATCTCCCTCTCCAGCTGCCAGCCCGACCTGCGTCCTGCGGGCAAGC
AGCGTGTGCCCACAGGCATGTCCTTCACCAACATCTACAGCACCCTGAGCGGCCAGGGCCGCCTCTAC
GACTACGGGCAGCGGTTTGTGCTGGGCATGGGCAGCTCGTCCATCGAGCTTTGTGAGCGGGAGTTCCA
GCAAGCAGGATGGCTATGTGCAGTTCGACAAGGCCAGCAAGGCTTCTGCTTCCTCCTCCCACCACTCC
GGGC

| | | | |
|---|---|---|---|
| NOV30f, 311346885 | | | |
| Protein Sequence | SEQ ID NO: 762 | 205 aa | MW at 22667.6kD |

TGSEIVHKEPASGREGEEHSTIKQLMMDRGEFQQDSVLKQLEVLKEEEKEFQNLKDPTNGYYSVNTFK
EHHSTPTISLSSCQPDLRPAGKQRVPTGMSFTNTYSTLSGQGRLYDYGQRFVLGMGSSSIELCEREFQ
RGSLSDSSSFLDTQCDSSVSSSGKQDGYVQFDKASKASASSSHHSQSSSQNSDPSRPLQRRMQTHVPG

| | | |
|---|---|---|
| NOV30g, 311333299 | SEQ ID NO: 763 | 1666 bp |
| DNA Sequence | ORF Start: at 2 | ORF Stop: end of sequence |

CACCGGATCCACCAACAAAGCCATCCCCGGAGGAAAGGAGACGTCGGTCACCATTGACATCCAGCACC
CTCCACTGGTCAACCTCTCGGTGGAGCCACAGCCAGTGCTGGAGGACAACGTCGTCACTTTCCACTGC
TCTGCAAAGGCCAACCCAGCTGTCACCCAGTACAGGTGGGCCAAGCGGGGCCAGATCATCAAGGAGGC
ATCTGGAGAGGTGTACAGGACCACAGTGGACTACACGTACTTCTCAGAGCCCGTCTCCTGTGAGGTGA
CCAACGCCCTGGGCAGCACCAACCTCAGCCGCACGGTTGACGTCTACTTTGGGCCCCGGATGACCACA
GAACCCCAATCCTTGCTCGTGGATCTGGGCTCTGATGCCATCTTCAGCTGCGCCTGGACCGGCAACCC
ATCCCTGACCATCGTCTGGATGAAGCGGGGCTCCGGAGTGGTCCTGAGCAATGAGAAGACCCTGACCC
TCAAATCCGTGCGCCAGGAGGACGCGGGCAAGTACGTGTGCCGGGCTGTGGTGCCCCGTGTGGGAGCC
GGGGAGAGAGAGGTGACCCTGACCGTCAATGGACCCCCCATCATCTCCAGCACCCAGACCCAGCACGC
CCTCCACGGCGAGAAGGGCCAGATCAAGTGCTTCATCCGGAGCACGCCGCCGCCGGACCGCATCGCCT
GGTCCTGGAAGGAGAACGTTCTGGAGTCGGGCACATCGGGGCGCTATACGGTGGAGACCATCAGCACC
GAGGAGGGCGTCATCTCCACCCTGACCATCAGCAACATCGTGCGGGCCGACTTCCAGACCATCTACAA
CTGCACGGCCTGGAACAGCTTCGGCTCCGACACTGAGATCATCCGGCTCAAGGAGCAAGGTTCGGAAA
TGAAGTCGGGAGCCGGGCTGGAAGCAGAGTCTGTGCCGATGGCCGTCATCATTGGGGTGGCCGTAGGA
GCTGGTGTGGCCTTCCTCGTCCTTATGGCAACCATCGTGGCGTTCTGCTGTGCCCGTTCCCAGAGAAA
TCTCAAAGGTGTTGTGTCAGCCAAAAATGATATCCGAGTGGAAATTGTCCACAAGGAACCAGCCTCTG
GTCGGGAGGGTGAGGAGCACTCCACCATCAAGCAGCTGATGATGGACCGGGGTGAATTCCAGCAAGAC
TCAGTCCTGAAACAGCTGGAGGTCCTCAAAGAAGAGGAGAAAGAGTTTCAGAACCTGAAGGACCCCAC
CAATGGCTACTACAGCGTCAACACCTTCAAAGAGCACCACTCAACCCCGACCATCTCCCTCTCCAGCT
GCCAGCCCGACCTGCGTCCTGCGGGCAAGCAGCGTGTGCCCACAGGCATGTCCTTCACCAACATCTAC
AGCACCCTGAGCGGCCAGGGCCGCCTCTACGACTACGGGCAGCGGTTTGTGCTGGGCATGGGCAGCTC
GTCCATCGAGCTTTGTGAGCGGGAGTTCCAGAGAGGCTCCCTCAGCGACAGCAGCTCCTTCCTGGACA
CGCAGTGTGACAGCAGCGTCAGCAGCAGCGGCAAGCAGGATGGCTATGTGCAGTTCGACAAGGCCAGC
AAGGCTTCTGCTTCCTCCTCCCACCACTCCCAGTCCTCGTCCCAGAACTCTGACCCCAGTCGACCCCT
GCAGCGGCGGATGCAGACTCACGTCCCCGGGGGC

| | | | |
|---|---|---|---|
| NOV30g, 311333299 | | | |
| Protein Sequence | SEQ ID NO: 764 | 555 aa | MW at 60511.3kD |

TGSTNKAIPGGKETSVTIDIQHPPLVNLSVEPQPVLEDNVVTFHCSAKANPAVTQYRWAKRGQIIKEA
SGEVYRTTVDYTYFSEPVSCEVTNALGSTNLSRTVDVYFGPRMTTEPQSLLVDLGSDAIFSCAWTGNP
SLTIVWMKRGSGVVLSNEKTLTLKSVRQEDAGKYVCPAVVPRVGAGEREVTLTVNGPPIISSTQTQHA
LHGEKGQIKCFIRSTPPPDRIAWSWKENVLESGTSGRYTVETISTEEGVISTLTISNIVRADFQTIYN
CTAWNSFGSDTEIIRLKEQGSEMKSGAGLEAESVPMAVIIGVAVGAGVAFLVLMATIVAFCCARSQRN
LKGVVSAKNDIRVEIVHKEPASGREGEEHSTIKQLMMDRGEFQQDSVLKQLEVLKEEEKEFQNLKDPT
NGYYSVNTFKEHHSTPTISLSSCQPDLRPAGKQRVPTGMSFTNIYSTLSGQGRLYDYGQRFVLGMGSS
SIELCEREFQRGSLSDSSSFLDTQCDSSVSSSGKQDGYVQFDKASKASASSSHHSQSSSQNSDPSRPL
QRRMQTHVPGG

| | | |
|---|---|---|
| NOV30h, CG55784-02 | SEQ ID NO: 765 | 891 bp |
| DNA Sequence | ORF Start: at 1 | ORF Stop: end of sequence |

AACAAAGCCATCCCCGGAGGAAAGGAGACGTCGGTCACCATTGACATCCAGCACCCTCCACTGGTCAA
CCTCTCGGTGGAGCCACAGCCAGTGCTGGAGGACAACGTCGTCACTTTCCACTGCTCTGCAAAGGCCA
ACCCAGCTGTCACCCAGTACAGGTGGGCCAAGCGGGGCCAGATCATCAAGGAGGCATCTGGAGAGGTG
TACAGGACCACAGTGGACTACACGTACTTCTCAGAGCCCGTCTCCTGTGAGGTGACCAACGCCCTGGG
CAGCACCAACCTCAGCCGCACGGTTGACGTCTACTTTGGGCCCCGGATGACCACAGAACCCCAATCCT
TGCTCGTGGATCTGGGCTCTGATGCCATCTTCAGCTGCGCCTGGACCGGCAACCCATCCCTGACCATC
GTCTGGATGAAGCGGGGCTCCGGAGTGGTCCTGAGCAATGAGAAGACCCTGACCCTCAAATCCGTGCG
CCAGGAGGACGCGGGCAAGTACGTGTGCCGGGCTGTGGTGCCCCGTGTGGGAGCCGGGGAGAGAGAGG
TGACCCTGACCGTCAATGGACCCCCCATCATCTCCAGCACCCAGACCCAGCACGCCCTCCACGGCGAG
AAGGGCCAGATCAAGTGCTTCATCCGGAGCACGCCGCCGCCGGACCGCATCGCCTGGTCCTGGAAGGA
GAACGTTCTGGAGTCGGGCACATCGGGGCGCTATACGGTGGAGACCATCAGCACCGAGGAGGGCGTCA
TCTCCACCCTGACCATCAGCAACATCGTGCGGGCCGACTTCCAGACCATCTACAACTGCACGGCCTGG
AACAGCTTCGGCTCCGACACTGAGATCATCCGGCTCAAGGAGCAAGGTTCGGAAATGAAGTCGGGAGC
CGGGCTG

| | | | |
|---|---|---|---|
| NOV30h, CG55784-02 | | | |
| Protein Sequence | SEQ ID NO: 766 | 297 aa | MW at 32432.3kD |

NKAIPGGKETSVTIDIQHPPLVNLSVEPQPVLEDNVVTFHCSAKANPAVTQYRWAKRGQIIKEASGEV
YRTTVDYTYFSEPVSCEVTNALGSTNLSRTVDVYFGPRMTTEPQSLLVDLGSDAIFSCAWTGNPSLTI
VWMKRGSGVVLSNEKTLTLKSVRQEDAGKYVCEAVVPRVGAGEREVTLTVNGPPIISSTQTQHALHGE
KGQIKCFIRSTPPPDRIAWSWKENVLESGTSGRYTVETISTEEGVISTLTISNIVRADFQTIYNCTAW
NSFGSDTEIIRLKEQGSEMKSGAGL

| | | |
|---|---|---|
| NOV30i, | | |
| SNP13376439 of | SEQ ID NO: 767 | 1806 bp |
| CG55784-01, | ORF Start: at 1 | ORF Stop: TAA at 1645 |
| DNA Sequence | SNP Pos: 1305 | SNP Change: C to T |

AACAAAGCCATCCCCGGAGGAAAGGAGACGTCGGTCACCATTGACATCCAGCACCCTCCACTGGTCAA
CCTCTCGGTGGAGCCACAGCCAGTGCTGGAGGACAACGTCGTCACTTTCCACTGCTCTGCAAAGGCCA
ACCCAGCTGTCACCCAGTACAGGTGGGCCAAGCGGGGCCAGATCATCAAGGAGGCATCTGGAGAGGTG
TACAGGACCACAGTGGACTACACGTACTTCTCAGAGCCCGTCTCCTGTGAGGTGACCAAAGCCCTGGG
CAGCACCAACCTCAGCCGCACGGTTGACGTCTACTTTGGGCCCCGGATGACCACAGAACCCCAATCCT
TGCTCGTGGATCTGGGCTCTGATGCCATCTTAAGCTGCGCCTGGACCGGCAACCCATCCCTGACCATC
GTCTGGATGAAGCGGGGCTCCGGAGTGGTCCTGAGCAATGAGAAGACCCTGACCCTCAAATCCGTGCG
CCAGGAGGACGCGGGCAAGTACGTGTGCCGGGCTGTGGTGCCCCGTGTGGGAGCCGGGGAGAGAGAGG
TGACCCTGACCGTCAATGGACCCCCCATCATCTCCAGCACCCAGACCCAGCACGCCCTCCACGGCGAG
AAGGGCCAGATCAAGTGCTTCATCCGGAGCACGCCGCCGCCGGACCGCATCGCCTGGTCCTGGAAGGA
GAACGTTCTGGAGTCGGGCACATCGGGGCGCTATACGGTGGAGACCATCAGCACCGAGGAGGGCGTCA
TCTCCACCCTGACCATCAGCAACATCGTGCGGGCCGACTTCCAGACCATCTACAACTGCACGGCCTGG
AACAGCTTCGGCTCCGACACTGAGATCATCCGGCTCAAGGAGCAAGGTTCGGAAATGAAGTCGGGAGC
CGGGCTGGAAGCAGAGTCTGTGCCGATGGCCGTCATCATTGGGGTGGCCGTAGGAGCTGGTGTGGCCT
TCCTCGTCCTTATGGCAACCATCGTGGCGTTCTGCTGTGCCCGTTCCCAGAGAAATCTCAAAGGTGTT
GTGTCAGCCAAAAATGATATCCGAGTGGAAATTGTCCACAAGGAACCAGCCTCTGGTCGGGAGGGTGA
GGAGCACTCCACCATCAAGCAGCTGATGATGGACCGGGGTGAATTCCAGCAAGACTCAGTCCTGAAAC
AGCTGGAGGTCCTCAAAGAAGAGGAGAAAGAGTTTCAGAACCTGAAGGACCCCACCAATGGCTACTAC
AGCGTCAACACCTTCAAAGAGCACCACTCAACCCCGACCATCTCCCTCTCCAGCTGCCAGCCCGACCT
GCGTCCTGCGGGTAAGCAGCGTGTGCCCACAGGCATGTCCTTCACCAACATCTACAGCACCCTGAGCG
GCCAGGGCCGCCTCTACGACTACGGGCAGCGGTTTGTGCTGGGCATGGGCAGCTCGTCCATCGAGCTT
TGTGAGCGGGAGTTCCAGAGAGGCTCCCTCAGCGACAGCAGCTCCTTCCTGGACACGCAGTGTGACAG
CAGCGTCAGCAGCAGCGGCAAGCAGGATGGCTATGTGCAGTTCGACAAGGCCAGCAAGGCTTCTGCTT
CCTCCTCCCACCACTCCCAGTCCTCGTCCCAGAACTCTGACCCCAGTCGACCCCTGCAGCGGCGGATG
CAGACTCACGTCTAAGGATCACACACCGCGGGTGGGGACGGGCCAGGGAAGAGGTCAGGGCACGTTCT
GGTTGTCCAGGGACTGTGGGGTACTTTACAGAGGACACCAGAATGGCCCACTTCCAGGACAGCCTCCC
AGCGCCTCTGCCACTGCCTTCCTTCGAAGCTCTGATCA

| | | | |
|---|---|---|---|
| Nov30i, | | | |
| SNP1 3376439 of | | | |
| CG55784-01, | SEQ ID NO: 768 | | MW at 59933.8kD |
| Protein Sequence | SNP Pos: 435 | 548 aa | SNP Change: Gly to Gly |

NKAIPGGKETSVTIDIQHPPLVNLSVEPQPVLEDNVVTFHCSAKANPAVTQYRWAKRGQIIKEASGEV
YRTTVDYTYFSEPVSCEVTKALGSTNLSRTVDVYFGPRMTTEPQSLLVDLGSDAILSCAWTGNPSLTI
VWMKRGSGVVLSNEKTLTLKSVRQEDAGKYVCRAVVPRVGAGEREVTLTVNGPPIISSTQTQHALHGE
KGQIKCFIRSTPPPDRIAWSWKENVLESGTSGRYTVETISTEEGVISTLTISNIVRADFQTIYNCTAW
NSFGSDTEIIRLKEQGSEMKSGAGLEAESVPMAVIIGVAVGAGVAFLVLMATIVAFCCARSQRNLKGV
VSAKNDIRVEIVHKEPASGREGEEHSTIKQLMMDRGEFQQDSVLKQLEVLKEEEKEFQNLKDPTNGYY
SVNTFKEHHSTPTISLSSCQPDLRPAGKQRVPTGMSFTNIYSTLSGQGRLYDYGQRFVLGMGSSSIEL
CEREFQRGSLSDSSSFLDTQCDSSVSSSGKQDGYVQFDKASKASASSSHHSQSSSQNSDPSRPLQRRM
QTHV

A ClustalW comparison of the above protein sequences yields the following sequence alignment shown in Table 30B.

TABLE 30B

Comparison of the NOV30 protein sequences.

| | |
|---|---|
| NOV30a | MKPFQLDLLFVCFFLFSQELGLQKRGCCLVLGYMAKDKFRRMNEGQVYSFSQQPQDQVVV |
| NOV30b | ------------------------------------------------------------ |
| NOV30c | ------------------------------------------------------------ |
| NOV30d | ------------------------------------------------------------ |
| NOV30e | ------------------------------------------------------------ |
| NOV30f | ------------------------------------------------------------ |
| NOV30g | ------------------------------------------------------------ |
| NOV30h | ------------------------------------------------------------ |
| NOV30a | SGQPVTLLCAIPEYDGFVLWIKDGLALGVGRDLSSYPQYLVVGNHLSGEHHLKILRAELQ |
| NOV30b | ------------------------------------------------------------ |
| NOV30c | ------------------------------------------------------------ |
| NOV30d | ------------------------------------------------------------ |
| NOV30e | ------------------------------------------------------------ |
| NOV30f | ------------------------------------------------------------ |
| NOV30g | ------------------------------------------------------------ |
| NOV30h | ------------------------------------------------------------ |
| NOV30a | DDAVYECQAIQAAIRSRPARLTVLVPPDDPVILGGPVISLRAGDPLNLTCHADNAKPAAS |
| NOV30b | ------------------------------------------------------------- |
| NOV30c | ------------------------------------------------------------- |
| NOV30d | ------------------------------------------------------------- |
| NOV30e | ------------------------------------------------------------- |
| NOV30f | ------------------------------------------------------------- |
| NOV30g | ------------------------------------------------------------- |
| NOV30h | ------------------------------------------------------------- |
| NOV30a | IIWLRKGEVINGATYSKTLLRDGKRESIVSTLFISPGDVENGQSIVCRATNKAIPGGKET |
| NOV30b | --------------------------------------------------NKAIPGGKET |
| NOV30c | ----------------------------------------------TGSTNKAIPGGKET |
| NOV30d | ----------------------------------------------TGSTNKAIPGGKET |
| NOV30e | ----------------------------------------------TGSTNKAIPGGKET |
| NOV30f | -------------------------------------------------TGSEIVHKEPA |
| NOV30g | ----------------------------------------------TGSTNKAIPGGKET |
| NOV30h | --------------------------------------------------NKAIPGGKET |
| NOV30a | SVTIDIQHPPLVNLSVEPQPVLEDNVVTFHCSAKANPAVTQYRWAKRGQIIKEASGEVYR |
| NOV30b | SVTIDIQHPPLVNLSVEPQPVLEDNVVTFHCSAKANPAVTQYRWAKRGQIIKEASGEVYR |
| NOV30c | SVTIDIQHPPLVNLSVEPQPVLEDNVVTFHCSAKANPAVTQYRWAKRGQIIKEASGEVYR |
| NOV30d | SVTIDIQHPPLVNLSVEPQPVLEDNVVTFHCSAKANPAVTQYRWAKRGQIIKEASGEVYR |
| NOV30e | SVTIDIQHPPLVNLSVEPQPVLEDNVVTFHCSAKANPAVTQYRWAKRGQIIKEASGEVYR |
| NOV30f | SGREGEEHSTIKQLMMDRGEFQQDSVLKQLEVLKEEEKEFQNLKDPTNGYYSVNTFKEHH |
| NOV30g | SVTIDIQHPPLVNLSVEPQPVLEDNVVTFHCSAKANPAVTQYRWAKRGQIIKEASGEVYR |
| NOV30h | SVTIDIQHPPLVNLSVEPQPVLEDNVVTFHCSAKANPAVTQYRWAKRGQIIKEASGEVYR |
| NOV30a | TTVDYTYFSEPVSCEVTNALGSTNLSRTVDVYFGPRMTTEPQSLLVDLGSDAIFSCAWTG |
| NOV30b | TTVDYTYFSEPVSCEVTKALGSTNLSRTVDVYFGPRMTTEPQSLLVDLGSDAILSCAWTG |
| NOV30c | TTVDYTYFSEPVSCEVTNALGSTNLSRTVDVYFGPRMTTEPQSLLVDLGSDAIFSCAWTG |
| NOV30d | TTVDYTYFSEPVSCEVTNALGSTNLSRTVDVYFGPRMTTEPQSLLVDLGSDAIFSCAWTG |
| NOV30e | TTVDYTYFSEPVSCEVTNALGSThLSRTVDVYFGPRMTTEPQSLLVDLGSDAIFSCAWTG |
| NOV30f | STPTISLSSCQPDLRPAGKQRVPTGMSFTNIYSTLSGQGR----LYDYGQRFVLGMGSS- |
| NOV30g | TTVDYTYFSEPVSCEVTNALGSTNLSRTVDVYFGPRMTTEPQSLLVDLGSDAIFSCAWTG |
| NOV30h | TTVDYTYFSEPVSCEVTNALGSTNLSRnTDVYFGPRMTTEPQSLLVDLGSDAIFSCAWTG |
| NOV30a | NPSLTIVWMKRGSGVVLSNEKTLTLKSVRQEDAGKYVCBAVVPRVGAGEREVTLTVNGPP |
| NOV30b | NPSLTIVWMKRGSGVVLSNEKTLTLKSVRQEDAGKYVCRAVVPRVGAGEREVTLTVNGPP |
| NOV30c | NPSLTIVWMKRGSGVVLSNEKTLTLKSVRQEDAGKYVCRAVVPRVGAGEREVTLTVNGPP |
| NOV30d | NPSLTIVWMKRGSGVVLSNEKTLTLKSVRQEDAGKYVCRAVVPRVGAGEREVTLTVNGPP |
| NOV30e | NPSLTIVWMKRGSGVVLSNEKTLTLKSVRQEDAGKYVCRAVVPRVGAGEREVTLTVNGPP |
| NOV30f | SIELCEREFQRGS---LSDSSSFLDTQCDSSVSSSGKQDGYVQFDKASKASASSSHHSQ- |
| NOV30g | NPSLTIVWMKRGSGVVLSNEKTLTLKSVRQEDAGKYVCRAVVPRVGAGEREVTLTVNGPP |
| NOV30h | NPSLTIVWMKRGSGVVLSNEKTLTLKSVRQEDAGKYVCRAVVPRVGAGEREVTLTVNGPP |
| NOV30a | IISSTQTQHALHGEKGQIKCFIRSTPPPDRIAWSWKENVLESGTSGRYTVETISTEEGVI |
| NOV30b | IISSTQTQHALHGEKGQIKCFIRSTPPPDRIAWSWKENVLESGTSGRYTVETISTEEGVI |
| NOV30c | IISSTQTQHALHGEKGQIKCFIRSTPPPDRIAWSWKENVLESGTSGRYTVETISTEEGVI |
| NOV30d | IISSTQTQHALHGEKGQIKCFIRSTPPPDRIAWSWKENVLESGTSGRYTVETISTEEGVI |
| NOV30e | IISSTQTQHALHGEKGQIKCFIRSTPPPDRIAWSWKENVLESGTSGRYTVETISTEEGVI |
| NOV30f | -S-SSQNSDPSRPLQRRMQTHVPGG----------------------------------- |
| NOV30g | IISSTQTQHALHGEKGQIKCFIRSTPPPDRIAWSWKENVLESGTSGRYTVETTSTEEGVI |
| NOV30h | IISSTQTQHALHGEKGQIKCFIRSTPPPDRIAWSWKENVLESGTSGRYTVETTSTEEGVI |
| NOV30a | STLTISNIVRADFQTIYNCTAWNSFGSDTEIIRLKEQ------------ESVPMAVIIGV |
| NOV30b | STLTISNIVPADFQTIYNCTAWNSFGSDTEIIRLKEQGSEMKSGAGLEAESVPMAVIIGV |
| NOV30c | STLTISNIVRADFQTIYNCTAWNSFGSDTEIIRLKEQ------------ESVPMAVIIGV |
| NOV30d | STLTISNIVRADFQTIYNCTAWNSFGSDTEIIRLKEQ------------ESVPMAVIIGV |
| NOV30e | STLTISNIVRADFQTIYNCTAWNSFGSDTEIIRLKEQGSEMKSGAGLEAESVPMAVIIGV |
| NOV30f | ------------------------------------------------------------ |
| NOV30g | STLTISNIVPADFQTIYNCTAWNSFGSDTEIIRLKEQGSEMKSGAGLEAESVPMAVIIGV |
| NOV30h | STLTISNIVHADFQTIYNCTAWNSFGSDTEIIRLKEQG-------------------SEM |
| NOV30a | AVGAGVAFLVLMATIVAFCCARSQRNLKGVVSAKNDIRVEIVHKEPASGREGEEHSTIKQ |
| NOV30b | AVGAGVAFLVLMATIVAFCCARSQRNLKGVVSAKNDIRVEIVHKEPASGREGEEHSTIKQ |
| NOV30c | AVGAGVAFLVLMATIVAFCCARSQRNLKGVVSAKNDIRVEIVHKEPASGREGEEHSTIKQ |
| NOV30d | AVGAGVAFLVLMATIVAFCCARSQHNLKGVVSAKNDIRVEPGG----------------- |
| NOV30e | AVGAGVAFLVLMATIVAFCCARSQRNLKGVVSAKNDIRVEPGG----------------- |
| NOV30f | ------------------------------------------------------------ |
| NOV30g | AVGAGVAFLVLMATIVAFCCARSQRNLKGVVSAKNDIRVEIVHKEPASGREGEEHSTIKQ |
| NOV30h | KSGAGL------------------------------------------------------ |
| NOV30a | LMMDRGEFQQDSVLKQLEVLKEEEKEFQNLKDPTNGYYSVNTFKEHHSTPTISLSSCQPD |
| NOV30b | LMMDRGEFQQDSVLKQLEVLKEEEKEFQNLKDPTNGYYSVNTFKEHHSTPTISLSSCQPD |
| NOV30c | LMMDRGEFQQDSVLKQLEVLKEEEKEFQNLKDPTNGYYSVNTFKEHHSTPTISLSSCQPD |
| NOV30d | ------------------------------------------------------------ |
| NOV30e | ------------------------------------------------------------ |
| NOV30f | ------------------------------------------------------------ |
| NOV30g | LMMDRGEFQQDSVLKQLEVLKEEEKEFQNLKDPTNGYYSVNTFKEHHSTPTISLSSCQPD |
| NOV30h | ------------------------------------------------------------ |
| NOV30a | LRPAGKQRVPTGMSFTNIYSTLSGQGRLYDYGQRFVLGMGSSSIELCEREFQRGSLSDSS |
| NOV30b | LRPAGKQRVPTGMSFTNIYSTLSGQGRLYDYGQRFVLGMGSSSIELCEREFQRGSLSDSS |
| NOV30c | LRPAGKQRVPTGMSFTNIYSTLSGQGRLYDYGQRFVLGMGSSSIELCEREFQRGSLSDSS |
| NOV30d | ------------------------------------------------------------ |
| NOV30e | ------------------------------------------------------------ |
| NOV30f | ------------------------------------------------------------ |
| NOV30g | LRPAGKQRVPTGMSFTNIYSTLSGQGRLYDYGQRFVLGMGSSSIELCEREFQRGSLSDSS |
| NOV30h | ------------------------------------------------------------ |
| NOV30a | SFLDTQCDSSVSSSGKQDGYVQFDKASKASASSSHHSQSSSQNSDPSRPLQRRMQTHV-- |
| NOV30b | SFLDTQCDSSVSSSGKQDGYVQFDKASKASASSSHHSQSSSQNSDPSRPLQRRMQTHV-- |
| NOV30c | SFLDTQCDSSVSSSGKQDGYVQFDKASKASASSSHHSQSSSQNSDPSRPLQRRMQTHVPG |
| NOV30d | ------------------------------------------------------------ |
| NOV30e | ------------------------------------------------------------ |
| NOV30f | ------------------------------------------------------------ |
| NOV30g | SFLDTQCDSSVSSSGKQDGYVQFDKASKASASSSHHSQSSSQNSDPSRPLQRRMQTHVPG |
| NOV30h | ------------------------------------------------------------ |
| NOV30a | - |
| NOV30b | - |
| NOV30c | G |
| NOV30d | - |
| NOV30e | - |
| NOV30f | - |
| NOV30g | G |
| NOV30h | - |
| NOV30a | (SEQ ID NO: 752) |
| NOV30b | (SEQ ID NO: 754) |
| NOV30c | (SEQ ID NO: 756) |
| NOV30d | (SEQ ID NO: 758) |
| NOV30e | (SEQ ID NO: 760) |
| NOV30f | (SEQ ID NO: 762) |
| NOV30g | (SEQ ID NO: 764) |
| NOV30h | (SEQ ID NO: 766) |

Further analysis of the NOV30a protein yielded the following properties shown in Table 30C.

TABLE 30C
Protein Sequence Properties NOV30a

| | |
|---|---|
| SignalP analysis: | Cleavage site between residues 24 and 25 |
| PSORT II analysis: | |

| PSG: | a new signal peptide prediction method | |
|---|---|---|
| | N-region: | length 7; pos. chg 1; neg. chg 1 |
| | H-region: | length 11; peak value 13.09 |
| | PSG score: | 8.69 |

| | |
|---|---|
| GvH: | von Heijne's method for signal seq. recognition |
| | GvH score (threshold: −2.1): −3.29 |
| | possible cleavage site: between 17 and 18 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 2 | | |
| | Number of TMS(s) for threshold 0.5: 1 | | |
| | INTEGRAL | Likelihood = | Transmembrane 524-540 |
| | | −10.40 | |

| | |
|---|---|
| PERIPHERAL | Likelihood = 1.54 (at 65) |

| | |
|---|---|
| | ALOM score: −10.40 (number of TMSs: 1) |
| MTOP: | Prediction of membrane topology (Hartmann et al.) |
| | Center position for calculation: 531 |
| | Charge difference: 5.0 C(3.0)-N(−2.0) |
| | C > N: C-terminal side will be inside |

>>>Caution: Inconsistent mtop result with signal peptide
>>> membrane topology: type 1b (cytoplasmic tail 524 to 766)

| MITDISC: | discrimination of mitochondrial targeting seq | | | |
|---|---|---|---|---|
| | R content: | 0 | Hyd Moment(75): | 8.25 |
| | Hyd Moment(95): | 6.93 | G content: | 0 |
| | D/E content: | 2 | S/T content: | 1 |

| | |
|---|---|
| | Score: −6.35 |
| Gavel: | prediction of cleavage sites for mitochondrial preseq |
| | cleavage site motif not found |

NUCDISC: discrimination of nuclear localization signals

| | |
|---|---|
| | pat4: none |
| | pat7: none |
| | bipartite: KRGCCLVLGYMAKDKFR at 24 |
| | content of basic residues: 10.2% |
| | NLS Score: 0.02 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: none
checking 63 PROSITE DNA binding motifs:

Leucine zipper pattern (PS00029): *** found ***
LLFVCFFLFSQELGLQKRGCCL at 8

none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 55.5 |
| COIL: | Lupas's algorithm to detect coiled-coil regions |
| | total: 0 residues |

Final Results (k = 9/23):

```
39.1%: nuclear
21.7%: cytoplasmic
17.4%: mitochondrial
  8.7%: vesicles of secretory system
  4.3%: vacuolar
  4.3%: peroxisomal
  4.3%: endoplasmic reticulum
```

>> prediction for CG55784-03 is nuc (k = 23)

A search of the NOV30a protein against the Geneseq database, a proprietary database that contains sequences published in patents and patent publication, yielded several homologous proteins shown in Table 30D.

TABLE 30D
Geneseq Results for NOV30a

| | | NOV30a | Identities/ | | |
|---|---|---|---|---|---|
| | | Residues/ | Similarities for | | |
| Geneseq | Protein/Organism/Length | Match | the Matched | | Expect |
| Identifier | [Patent #, Date] | Residues | Region | | Value |
| ABG96273 | Human immunoglobulin superfamily | 1 . . . 766 | 766/766 | (100%) | 0.0 |
| | protein IGSFP-11 - Homo sapiens, 766 | 1 . . . 766 | 766/766 | (100%) | |
| | aa. [WO200272794-A2, 19-SEP-2002] | | | | |
| ABU58692 | Human PRO polypeptide #293 - Homo | 1 . . . 557 | 554/569 | (97%) | 0.0 |
| | sapiens, 600 aa. [US2003027272-A1, | 1 . . . 569 | 555/569 | (97%) | |
| | 06-FEB-2003] | | | | |
| ABU57223 | Human PRO polypeptide #293 - Homo | 1 . . . 557 | 554/569 | (97%) | 0.0 |
| | sapiens, 600 aa. [US2003027280-A1, | 1 . . . 569 | 555/569 | (97%) | |
| | 06-FEB-2003] | | | | |
| ABU56228 | Human secreted/transmembrane | 1 . . . 557 | 554/569 | (97%) | 0.0 |
| | protein, PRO19814 - Homo sapiens, | 1 . . . 569 | 555/569 | (97%) | |
| | 600 aa. [US2003022298-A1, 30-JAN- | | | | |
| | 2003] | | | | |
| ABU10802 | Human secreted/transmembrane | 1 . . . 557 | 554/569 | (97%) | 0.0 |
| | protein #293 - Homo sapiens, 600 aa. | 1 . . . 569 | 555/569 | (97%) | |
| | [US2002127584-A1, 12-SEP-2002] | | | | |

In a BLAST search of public sequence databases, the NOV30a protein was found to have homology to the proteins shown in the BLASTP data in Table 30E.

TABLE 30E
Public BLASTP Results for NOV30a

| | | NOV30a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8IZU9 | NPEH2 - Homo sapiens (Human), | 1 . . . 766 | 766/778 (98%) | 0.0 |
| | 778 aa. | 1 . . . 778 | 766/778 (98%) | |
| Q96JG0 | Hypothetical protein KIAA1867 - | 1 . . . 766 | 766/778 (98%) | 0.0 |
| | Homo sapiens (Human), 779 aa | 2 . . . 779 | 766/778 (98%) | |
| | (fragment). | | | |
| AAO41835 | Membrane protein mKirre - Mus | 1 . . . 766 | 750/766 (97%) | 0.0 |
| | musculus (Mouse), 766 aa. | 1 . . . 766 | 762/766 (98%) | |
| Q8BR86 | Weakly similar to NEPH1 - Mus | 1 . . . 766 | 750/778 (96%) | 0.0 |
| | musculus (Mouse), 778 aa. | 1 . . . 778 | 762/778 (97%) | |
| Q8BGQ5 | Weakly similar to NEPH1 - Mus | 1 . . . 591 | 575/591 (97%) | 0.0 |
| | musculus (Mouse), 612 aa. | 1 . . . 591 | 588/591 (99%) | |

PFam analysis predicts that the NOV30a protein contains the domains shown in the Table 30F.

TABLE 30F
Domain Analysis of NOV30a

| | | Identities/ | |
|---|---|---|---|
| | | Similarities | |
| Pfam | NOV30a | for the | Expect |
| Domain | Match Region | Matched Region | Value |
| ig | 62 . . . 129 | 14/72 (19%) | 0.00022 |
| | | 41/72 (57%) | |
| ig | 163 . . . 229 | 13/69 (19%) | 0.0045 |
| | | 43/69 (62%) | |
| ig | 349 . . . 400 | 15/55 (27%) | 1.6e−06 |
| | | 39/55 (71%) | |
| ig | 433 . . . 501 | 12/70 (17%) | 0.31 |
| | | 45/70 (64%) | |

### Example 31

The NOV31 clone was analyzed, and the nucleotide and encoded polypeptide sequences are shown in Table 31A.

TABLE 31A

NOV31 Sequence Analysis

| | | |
|---|---|---|
| NOV31a, CG55790-02 | SEQ ID NO: 769 | 8250 bp |
| DNA Sequence | ORF Start: ATG at 4 | ORF Stop: TAG at 1423 |

ACCATGCGGCTGGGCAGTCCTGGACTGCTCTTCCTGCTCTTCAGCAGCCTTCGAGCTGATACTCAGGA
GAAGGAAGTCAGAGCGATGGTAGGCAGCGACGTGGAGCTCAGCTGCGCTTGCCCTGAAGGAAGCCGTT
TTGATTTAAATGATGTTTACGTATATTGGCAAACCAGTGAGTCGAAAACCGTGGTGACCTACCACATC
CCACAGAACAGCTCCTTGGAAAACGTGGACAGCCGCTACCGGAACCGAGCCCTGATGTCACCGGCCGG
CATGCTGCGGGCGACTTCTCCCTGCGCTTGTTCAACGTCACCCCCCCAGGACGAGCAGAAGTTTCACT
GCCTGGTGTTGAGCCAATCCCTGGGATTCCAGGAGGTTTTGAGCGTTGAGGTTACACTGCATGTGGCA
GCAAACTTCAGCGTGCCCGTCGTCAGCGCCCCCCACAGCCCCTCCCAGGATGAGCTCACCTTCACGTG
TACATCCATAAACGGCTACCCCAGGCCCAACGTGTACTGGATCAATAAGACGGACAACAGCCTGCTGG
ACCAGGCTCTGCAGAATGACACCGTCTTCTTGAACATGCGGGGCTTGTATGACGTGGTCAGCGTGCTG
AGGATCGCACGGACCCCCAGCGTGAACATTGGCTGCTGCATAGAGAACGTGCTTCTGCAGCAGAACCT
GACTGTCGGCAGCCAGACAGGAAATGACATCGGAGAGAGAGACAAGATCACAGAGAATCCAGTCAGTA
CCGGCGAGAAAAACGCGGCCACGTGGAGCATCCTGGCTGTCCTGTGCCTGCTTGTGGTCGTGGCGGTG
GCCATAGGCTGGGTGTGCAGGGACCGATGCCTCCAACACAGCTATGCAGGTGCCTGGGCTGTGAGTCC
GGAGACAGAGCTCACTGGTGAGTTTGCCGTGGGAAGCAGCAGGTTCTGGGGGGCCCAGGGGAGGCTTG
GCTGCCAGCTGTCTTTCAGAGTTTCAAAAAACTTTCAAAAGGCAAAAGTCCCTTGCCTTGAACAACTG
TTGTTCCTGGAGACGCAGCGAAGCCCTCGATGGTGCGCATGGCATTTCCTGCAGCCTCCCCTTGGCAT
GGGATGGCATCCTGGTGTGCACTTTGTCACACTGCGATGGGATTTTCCCAACATGCACAGAAGCAGAG
AGACGAGTGCTAGACCCCCGCGCTCCCCAGTGCCCAGCCCCGACCAGGGTGTCCAGGGCGGGTCCAGG
CACCGGCGCCCAGCCCCCATGGGGTGTCCGGAGTGGGTCCAGGCACCGGCGCCCAGCCCCCGTGGGGT
GTCCAGGGCGGGTCCAGGCACCGGCGCCCAGCCCCTGTGGGGTGTCCGGAGTGGGTCCGGGCACCGCC
AGCTTCTCTCTGTGGCAGCCACTCCTGCAGCTCTCGTTTGCCCCTCAGTTCCAGGAGCAACATAGATG
TGGATTCCTGTCCAATTTGGGAAAAATGTCCACACACGGTCACCCACCTGGCAGGTGCCTCTGGCTGC
AAGGGGCGCTGGGCTTCGCAGGCAGGCCAGCCGGGCTCCCCGCCATGGGCCAGGATCCCCTCCGAGCG
CTGTTTGCCGCCCAGGAGAAGGGGTTCCCCGGGGACAGTGGGCTCAGGGTGTGCGCAGCCACCATGCT
GTGGTGTCACCTGTGGACCCAGGCGAGCTGATGGCCGACCGCAGAAACGCACTTCCAAGGCCAGGTCG
GCCCATCCAGATGATGCAGGAACACAGCTTGCTAAAAACACGGCCGGCCTGTTCCCGTCGGAGCCAGT
CGAAGTTCCCTGAACAGGCCGCTGTTTCCGAAGCTTTAAACCCTGTGTTTCCACCAAGCTGAGTCCTG
AGAAAACCGACGTCTGCCTGCAGAAGGGAAAGGGGTGCTTCATGTTCCTCTCTCTCCTTCATCTCCCT
TCCAAGGCCACGTTTGACCGGAGCTCACCGCCCAGAGCGTGGACAGGGCTTCCGTGAGACGCCACCGT
GAGAGGCCAGGTGGCAGCTTGAGCATGGACTCCCAGACTGCAGGGGAGCACTTGGGGCAGCCCCCAGA
AGGACCACTGCTGGATCCCAGGGAGAACCTGCTGGCGTTGGCTGTGATCCTGGAATGAGGCCCTTTCA
AAAGCGTCATCCACACCAAAGGCAAATGTCCCCAAGTGAGTGGGCTCCCCGCTGTCACTGCCAGTCAC
CCACAGGAAGGGACTGGTGATGGGCTGTCTCTACCCGGAGCGTGCGGGATTCAGCACCAGGCTCTTCC
CAGTACCCCAGACCCACTGTGGGTCTTCCCGTGGGATGCGGGATCCTGAGACCGAAGGGTGTTTGGTT
TAAAAAGAAGACTGGGCGTCCGCTCTTCCAGGACGGCCTCTGTGCTGCTGGGGTCACGCGAGGCTGTT
TGCAGGGGACACGGTCACAGGAGCTCTTCTGCCCTGAACGCTCCCAACCTGCCTCCCGCCCGGAAGCC
ACAGGACCCACTCATGTGTGTGCCCACAAGTGTAGTTAGCCGTCCACACCGAGGAGCCCCCGGAAGTC
CCCACTGGGCTTCAGTGTCCTCTGCCACATTCCCTGGGAGGAACAATGTCCCTCGGCTGTTCCGGTGA
AAAGTTGAGCCACCTTTGGAAGACGCACGGGTGGAGTTTGCCAGAAGAAAGGCTGTGCCAGGGCCGTG
TTTGGCTACAGGGGCTGCCGGGGCTCTTGGCTCTGCAGCGAGAAAGACACAGCCCAGCAGGGCTGGAG
ACGCCCATGTCCAGCAGGCGCAGGCCTGGCAACACGGTCCCCAGAGTCCTGAGCAGCAGTTAGGTGCA
TGGAGAGGGTATCACCTGGTGGCCACAGTCCCCCTTCTCACCTCAGCAATGATCCCCAAAGTGAGAGC
TGGCTCCCCCGGCCCCCACCACCCTCAGCAGCCCCACCCCACTCAACCCTGAGGGTCCCCAGGGTCCT
GATGAAGACCTCCGACCCCAGCGCCAGGCTCCTCGGAGCCCAACAGTCCCAAGGGGGCAGGAGACGGG
GTGGTCCAGTGCTGAGGGGTACAGCCCTGGGCCCTGACCAGCCCCGGCACCTGCCATGCTGGTTCCCC
GAATGAATCAGCTGCTGACTGTCTCCAGAAGGGCTGGAAAGGATGCTGCCAGGTGACCCGAGGTGCAC
TCGCCCCAGGGAGATGGAGTAGACAGCCTGGCCTGGCCCTCGGGACACATTGTCTGCCCCGGGGCTAT
GGGCAAATGCCCCTCCTTCTTACTTCCCAGAATCCCCTGACATTCCCAGGGTCAGCCAGGACCTGTTA
AAGCCCTGGTCACTTGGAACTGACAGCTGTGTGAGGCCTGCACTTCTCAGACCCAGACTTAGAACAAA
AGGAGGAGTGAGGACTCAAGGCTACAATGAGGTTCCAGTACTTGTTACAAGAAATTGGTTTTCTGCAA
AAGATGTGTGGTGTTTATAAAAGAACAGTTGTCATCACCGGGCATTGATTGGCAGGGACAAGGAGCTG
CTTGGGTGTGGAAAGTTGGGGCGTTGGAAAGTGGGCTGTGGTGCCCATTTGCAGTGACTGTGAAGTGA
CTCCAGGACGGACCTGCGGGGGCACCCAGAGGTCCTAAGCCCCAGGACTGAGGGTCGTGCATCACCAC
TCGGGTGTCCCGGGAGGTGCCCTGGGCCCGGGGACCTCACAGGCAGGACGGCGACACTAATGCAGGGA
GAGGGAGTCTGGCCCCAGCTTTTCCTATCAGAGGCGATTTTCCTTCACCAGGGGATGGGCAGGAAAGA
GGCAGGGGCCCCAGAAGCTTCTGTCCCTCATGCCTGAGGGCACGGGGGACACTTGGAGGCTGCTGTCA
CCACTGTGCGTCCAAGGCCATGCTCTCTGCGGGTCAGTGCCTGAGTCTCGCCTCCCTGCTGGTCCCTG
AAGCCCCCTCAGAAGCCCTGCCTGTCACGTCGGCATTTGTGAGACCTACCCTGTAACGCCTGCCCCTC
TCAGCCCAACATCAGCTTCCTCTTTCTCCCTTGCTGTAGACAGGCTGGATTCCAGTGTTGGGACAGCC
ATCTCCAGAAACCTGACTTAAGAGAGTAAGATGCAAATCGTGCCTGTATCCAGTGGCTTTGGTGGGTC
CAGGGAGTCTTGGGCACAGCCAGCTCAGCTGTCTGTGGTATGAGCAGGAACAGGTGCCACTCCTGCTC
AGGGGACCCTGCCCTACACCAGGCTGTTCCGTCCCCCTGGAGGACATGGGGCCAGGTCTGGAGGCATT
TTGGGTTGTCACAGCTGGGGGCTGTTCCTCGGCTTCAGCGGGTGGAAGCCTCAGATGCTGTTCAACAT
CTTCTGGACACGGGAGGCCCCGACAGAGAGAAGCGTCCACCCGCAAGTCCACAGTCTGAGGTCTCCCC
TCAGAGACCCTGCCCTGCACACCCACCTCCAGCCAAAGGTCCTGCCTGCCCCAGGGCTCAGGGGAACC
TTGCCGGTCTGTGGAACAGGAGAGGGGACTCTCGCCAGCTGCACCACCCTGCACGTAGTAGGTGTGCG
GTAAACATCCACCAGGGAGGCTCCAGTCAAGGCTGGCAGATGGGGCGGTCCATCCCTAGGGCAGGTGA
CAGAAGGGAAAAGGCTGCCTGCTGGCCCCCGAGCCAGGTAGCACATGCTTGTGCCTCAGTTTCCCCTC
CTGTAAAGTGAGGCGCTGGATCCAGGTTCTGTCTACTGGGCTCTGCAGCTTGGACGCTCCTAAGACCA
AGCGACCCACCCTGGGGAGGGCAGCTATGGCTTTGGAATAGCTGTCCAGGCCCGGGTGCCTCCAAGAC
GGCCACCACACCCTGCCTGTGCTGCAGGGGTGCAGGGGTAAGGGGCAAGACTCCAGAGGCCTCCTCTC
TGCATCTCCTTGTCTTCAGTGGCCGGAGGTGAGGCCTGAGCTCAGGGGAGGGGCTTCTGCCACGAACC
CTATGGCGGGGCACAGCACACTTTTCCCAGGGAGGACCCCTGGGCCCCCTGCATTATCCCCAGCGGAC
TGTGGGGTCACCTTCCAAGAGCGACATTGAGAAGCTCCAGCTCTAGGAGTGTGCAGACTCTTAACCAG
GCAGGCCCAGGCCCTGGGGCACACAAAGGCGGGGCCTGCTCTCCCCAGCTGCCCCTGCCAATGGGGGC
TGGACTGTCCTACCCTCCTCCCTTCTACCTCCCCACTGTCTTCCCTCTCCACTGTCACCACTGCCTCC
CTCTTCCACTGTCCTCCATGCACTGCCCTCCCTCCACCTTCCCCCACCCCCACCACTCCCCATGCTGT
CCCCAGGCTCCCCCCGCTCTCCCCCCTCCCCACTGTCCCCCTCCCCATGCTGTACCCAGCTCACCCCC
CTCTCCCCTCTCCCCACTGTCCCCCCTCCCACTCCCCATGCTGTCCCCAGCTCACCCTACATGGACTT
GGCGATGTCCTTCCATGGCTCACCGGTCTGAATTTCCATGATGAGCCGGGCCTGCAGCTTTGCTCCCC
TATCCCTGCCCAGGCTGCAGCTGTCCATGCAGGGAGCGAGCTCCAGCACCTGCGGAGTCCTTCCGTGC
GGGCCTCTCCGTGCCACAGCAGCCAGGGACCTCAGGTGCCTGTGCATGACACCACCGCCCATCCTCAT
CCTGAGCCAGCCTCTCAGGATCAGGACTTGGTTTGGCGGCGTTAACCTTAGAGCCTGCAAGGGGCTTC
CTCCTGGTGGGTCTGGCCGTAGCCTGGGGAGGCCACAGCTCCAGGCCACTCCAGACCTCCCTTCCTCT
GGGCCTTCCATGTGGTGGCAACCACCGCAGCTGTAAGGGAGGGAAAATGGAGCGTTTGTTCTCGGGCT
GGGCTGGGGTCTGGGGGAAGCCATGGGCGTGAAGACTGGAGTATTATTTGATGGAGAAGCGGCCACTC
CTGGAGACCGGCGGCAAACACAGAAGCACAGCGTGGAAGGTGCTGGTGTCAGCCCACACGGGTGATGG
AGCCCCAGACACAATGCATTCCTGTTGCCCTCGCCCTCAGCCAGGCCCCACGCAGGCCAGGGAGCACT
GGCAAAGCTTGGCAACCCTCTGGGGGCCAGCCTTCATCCAGGCCGAAGGTGGTCAGTGGCCCACCATG
GCCCAGGTAGAAAACTCACGGATTAAGATTTCATGCCCGACTCCAAAGGCAAGAGACTTTATTATTTT
ATTTTTTTTGAGCCAGAGTATCGCTCTGTCACCTAGGCTGGAGTGCAATCTCTGCTCATTGCAACATC
TGCCTCCCGAACTCAAGCAATTCTGCCTCAGCCTCCCAAGTAGCTGGGATTACAGGTGTGCGCCACCA
TGCCCAGGTAATTGTATTTTTAGTAGAGACAGGGTTTCACCATGTTGGTCAGGCTGGTTTCAAACTCC
TGACCTCAAATGATCTGCCCACCTCGACCTCCCAAAGTGCTGGGATTACAGGTGCGAGCCACCGCACC
TGGCTACCAGACACTTCAGAGTTACAGGTTAGTTTTTCTTTTTCTTTTATTTTTTTTTTTTTGGCGGA
GGTGCAGGGGGAGTTAAACAAACAAACAAAATAAACAGGCCGGGTGCGGTGGCTCATGCCTGTAATCC
CAGCACTTTAGGAGGCCTAGGTGGGTGGATCACGAGATCAGGGGTTCAAGACCAGCCTGGCCGAGATG
GTAAAACCCCGTCTCCACTAAAAATACAAAAATTGGCCAGGCACGGTGGCTCACACCTGTAATCCCAG
TACTTTGGGAGGCTGAGGTGGGCAGATCACCTGAGGTCAGGAGTTCAAGACCAACCTGACCAACATGG
AGAAACCCCATCTCTACTAAAAATACAkAATTAGCCAGGTGTGGTGGTGCATGCCTGTAATTCCAGCT
GCCATTGCACTCCAGCCTGGGAACAAGAGCGAAACTCTGACTAAAAAAGAAAGAAAGAAAGAAAAAAA
TTAGTTGGGCACGGTGGCAGGCGCCTGTAATCCCAGGTACTCAGGAGGCTGAGGCAGGAGAATTGCTT
GAACCCGGGAGGCAGAGGTCGCAGTGAGCCGAGATTGCACCACTGCCCTCCAGCCTGGGTGACAGAGC
AAGACTCCGTCTCAAAAAAAAAAAAAAAAAAAATTGGATACATTGTAATACCTCAAATACTTGTAAGT
GAAGCACCCCAGTTCCCATAGAGCTGCCGCACTCAGAGGCTTCTGTAACCTGCCTGCTCCCAGCATTC
TATTTAGGGTCTGGTATGTCCAGAATTTGCAGACACAGCAATTCCTGCAGCAGCAGTGCACCATGTGC
AAGGGGCCCCATGACCAGCCCACTGTGAGCTCACACGTGATGACTGAGGCTTCTTCACACAGCAGGGC
TCTGGGTGTGATACCCAGGGCACACGCGTTTGCACAGGCACAGGCCACACAAGTTCTCACATGCTCAG
CCCCATAAGCCGTGCTGGACAGGCATGGCCATTTACACCCAGGATCCTGCTGAGAACAGCAACCAACT
CACCACCCTCGCATCATGATCCTTGCCACACAGGGGCTCTGGTGGCTTTGGTGGCCTGGGCTGTGGCT
CTGCTGCCAGCCACCTTGAGTGAAGATCCGGGTTCTCTGGGTGCTACTCAGCTGCTATGTGGGGAGCT
CTGTCTGCATACAGACTTCTCAGGGGAGTTCTCAGCTTGGACCCTTATCTCCCCAGAATCCTGGAACC
TGCTCCTTCTGCTCTCGTGACTGACTGTGTTCTCTATGCAACTTCCAATAAAACCTCTTCATTTGAAA
GGAAAAAAGTCTGCATTATCTGTTTAGGAAGGGAGAGAGTTCATATTGCAATCTTTTTTTTTTTAATT
AAAATAATCTCAGCCTGGGCAACATGGTGAGACCCCATCTCTGTAAAACATTTTTAAAAAATTAGCCG
GGTATGGTGGCGCACACTTGTAGTCCCAGCTACTCAGGAGGCTGAAGCGGGAGGATCCATTGAACCTG
AGAAGTCGAAGCTGCAGTGAGCTGTGATTGTGCCACTGTACTCCAGCCTGGACAACAGAGTGAGACGC
CGTCTCAAATAAATAAATACAT

| | | | |
|---|---|---|---|
| NOV31a, CG55790-02 | | | |
| Protein Sequence | SEQ ID NO: 770 | 1473 aa | MW at 51924.6kD |

MRLGSPGLLFLLFSSLEADTQEKEVRAMVGSDVELSCACPEGSRFDLNDVYVYWQTSESKTVVTYHIP
NSSLENVDSRYRNRALMSPAGMLRGDFSLRLFNVTPQDEQKFHCLVLSQSLGFQEVTLSVEVTLHVAA
NFSVPVVSAPHSPSQDELTFTCTSINGYPRPNVYWINKTDNSLLDQALQNDTVFLNNRGLYDVVSVLR
IARTPSVNIGCCIENVLLQQNLTVGSQTGNDIGERDKITENPVSTGEKNAATWSILAVLCLLVVVAVA
TGWVCRDRCLQHSYAGAWAVSPETELTGEFAVGSSRFWGAQGRLGCQLSFRVSKNFQKAKVPCLEQLL
FLETQRSPRWCAWHFLQPPLGMGWHPGVHFVTLRWDFPNMHRSRETSARPPRSPVPSPDQGVQGGSRH
RRPAPMGCPEWVQAPAPSPRGVSPAGPGTGAQPLWGVRSGSGHRQLLSVAATPAALVCPSVPGAT

| | | |
|---|---|---|
| NOV31b, 258668431 | SEQ ID NO: 771 | 1393 bp |
| DNA Sequence | ORF Start: at 2 | ORF Stop: end of sequence |

CACCAGATCTCTTCGAGCTGATACTCAGGAGAAGGAAGTCAGAGCGATGGTAGGCAGCGACGTGGAGC
TCAGCTGCGCTTGCCCTGAAGGAAGCCGTTTTGATTTAAATGATGTTTACGTATATTGGCAAACCAGT
GAGTCGAAAACCGTGGTGACCTACCACATCCCACAGAACAGCTCCTTGGAAAACGTGGACAGCCGCTA
CCGGAACCGAGCCCTGATGTCACCGGCCGGCATGCTGCGGGGCGACTTCTCCCTGCGCTTGTTCAACG
TCACCCCCCAGGACGAGCAGAAGTTTCACTGCCTGGTGTTGAGCCAATCCCTGGGATTCCAGGAGGTT
TTGAGCATTGAGGTTACACTGCATGTGGCAGCAAACTTCAGCGTGCCCGTCGTCAGCGCCCCCCACAG
CCCCTCCCAGGATGAGCTCACCTTCACGTGTACATCCATAAACGGCTACCCCAGGCCCAACGTGTACT
GGATCAATAAGACGGACAACAGCCTGCTGGACCAGGCTCTGCAGAATGACACCGTCTTCTTGAACATG
CGGGGCTTGTATGACGTGGTCAGCGTGCTGAGGATCGCACGGACCCCCAGCGTGAACATTGGCTGCTG
CATAGAGAACGTGCTTCTGCAGCAGAACCTGACTGTCGGCAGCCAGACAGGAAATGACATCGGAGAGA
GAGACAAGATCACAGAGAATCCAGTCAGTACCGGCGAGAAAAACGCGGCCACGTGGAGCATCCTGGCT
GTCCTGTGCCTGCTTGTGGTCGTGGCGGTGGCCATAGGCTGGGTGTGCAGGGACCGATGCCTCCAACA
CAGCTATGCAGGTGCCTGGGCTGTGAGTCCGGAGACAGAGCTCACTGGTGAGTTTGCCGTGGGAAGCA
GCAGGTTCTGGGGGGCCCAGGGGAGGCTTGGCTGCCAGCTGTCTTTCAGAGTTTCAAAAAACTTTCAA
AAGGCAAAAGTCCCTTGCCTTGAACAACTGTTGTTCCTGGAGACGCAGCGAAGCCCTCGATGGTGCGC
ATGGCATTTCCTGCAGCCTCCCCTTGGCATGGGATGGCATCCTGGTGTGCACTTTGTCACACTGCGAT
GGGATTTTCCCAACATGCACAGAAGCAGAGAGACGAGTGCTAGACCCCCGCGCTCCCCAGTGCCCAGC
CCCAACCAGGGTGTCCAGGGCGGGTCCAGGCACCGGCGCCCAGCCCCCATGGGGTGTCCGGAGTGGGT
CCAGGCACCGGCGCCCAGCCCCCGTGGGGTGTCCAGGGCGGGTCCAGGCACCGGCGCCCAGCCCCTGT
GGGGTGTCCGGAGCGGGTCCGGGCACCGCCAGCTTCTCTCTGTGGCAGCCACTCCTGCAGCTCTCGTT
TGCCCCTCAGTTCCAGGAGCAACACTCGAGGGC

| | | | |
|---|---|---|---|
| NOV31b, 258668431 | | | |
| Protein Sequence | SEQ ID NO: 772 | 464 aa | MW at 50961.4kD |

TRSLRADTQEKEVRAMVGSDVELSCACPEGSRFDLNDVYVYWQTSESKTVVTYHIPQNSSLENVDSRY
RNRALMSPAGMLRGDFSLRLFNVTPQDEQKFHCLVLSQSLGFQEVLSIEVTLHVAANFSVPVVSAPHS
PSQDELTFTCTSINGYPRPNVYWINKTDNSLLDQALQNDTVFLNNRGLYDVVSVLRIARTPSVNIGCC
IENVLLQQNLTVGSQTGNDIGERDKITENPVSTGEIGAATWSILAVLCLLVVVAVAIGIWCRDRCLQH
SYAGAWAVSPETELTGEFAVGSSRFWGAQGRLGCQLSFRVSKNFQKAKVPCLEQLLFLETQRSPRWCA
WHFLQPPLGMGWHPGVHFVTLRWDFPNMHRSRETSARPPRSPVPSPNQGVQGGSRHRRPAPMGCPEWV
QAPAPSPRGVSRAGPGTGAQPLWGVRSGSGHRQLLSVAATPAALVCPSVPGATLEG

| | | |
|---|---|---|
| NOV31c, 309303509 | SEQ ID NO: 773 | 1393 bp |
| DNA Sequence | ORF Start: at 2 | ORF Stop: end of sequence |

CACCAGATCTCTTCGAGCTGATACTCAGGAGAAGGAAGTCAGAGCGATGGTAGGCAGCGACGTGGAGC
TCAGCTGCGCTTGCCCTGAAGGAAGCCGTTTTGATTTAAATGATGTTTACGTATATTGGCAAACCAGT
GAGTCGAAAACCGTGGTGACCTACCACATCCCACAGAACAGCTCCTTGGAAAACGTGGACAGCCGCTA
CCGGAACCGAGCCCTGATGTCACCGGCCGGCATGCTGCGGGGCGACTTCTCCCTGCGCTTGTTCAACG
TCACCCCCCAGGACGAGCAGAAGTTTCACTGCCTGGTGTTGAGCCAATCCCTGGGATTCCAGGAGGTT
TTGAGCATTGAGGTTACACTGCATGTGGCAGCAAACTTCAGCGTGCCCGTCGTCAGCGCCCCCCACAG
CCCCTCCCAGGATGAGCTCACCTTCACGTGTACATCCATAAACGGCTACCCCAGGCCCAACGTGTACT
GGATCAATAAGACGGACAACAGCCTGCTGGACCAGGCTCTGCAGAATGACACCGTCTTCTTGAACATG
CGGGGCTTGTATGACGTGGTCAGCGTGCTGAGGATCGCACGGACCCCCAGCGTGAACATTGGCTGCTG
CATAGAGAACGTGCTTCTGCAGCAGAACCTGACTGTCGGCAGCCAGACAGGAAATGACATCGGAGAGA
GAGACAAGATCACAGAGAATCCAGTCAGTACCGGCGAGAAAAACGCGGCCACGTGGAGCATCCTGGCT
GTCCTGTGCCTGCTTGTGGTCGTGGCGGTGGCCATAGGCTGGGTGTGCAGGGACCGATGCCTCCAACA
CAGCTATGCAGGTGCCTGGGCTGTGAGTCCGGAGACAGAGCTCACTGGTGAGTTTGCCGTGGGAAGCA
GCAGGTTCTGGGGGGCCCAGGGGAGGCTTGGCTGCCAGCTGTCTTTCAGAGTTTCAAAAAACTTTCAA
AAGGCAAAAGTCCCTTGCCTTGAACAACTGTTGTTCCTGGAGACGCAGCGAAGCCCTCGATGGTGCGC
ATGGCATTTCCTGCAGCCTCCCCTTGGCATGGGATGGCATCCTGGTGTGCACTTTGTCACACTGCGAT
GGGATTTTCCCAACATGCACAGAAGCAGAGAGACGAGTGCTAGACCCCCGCGCTCCCCAGTGCCCAGC
CCCGACCAGGGTGTCCAGGGCGGGTCCAGGCACCGGCGCCCAGCCCCCATGGGGTGTCCGGAGTGGGT
CCAGGCACCGGCGCCCAGCCCCCGTGGGGTGTCCAGGGCGGGTCCAGGCACCGGCGCCCAGCCCCTGT
GGGGTGTCCGGAGCGGGTCCGGGCACCGCCAGCTTCTCTCTGTGGCAGCCACTCCTGCAGCTCTCGTT
TGCCCCTCAGTTCCAGGAGCAACACTCGAGGGC

| | | | |
|---|---|---|---|
| NOV31c, 309303509 | | | |
| Protein Sequence | SEQ ID NO: 774 | 464 aa | MW at 50962.4kD |

TRSLRADTQEKEVRAMVGSDVELSCACPEGSRFDLNDVYVYWQTSESKTVVTYHIPQNSSLENVDSRY
RNRALMSPAGMLRGDFSLRLFNVTPQDEQKFHCLVLSQSLGFQEVLSIEVTLHVAANFSVPVVSAPHS
PSQDELTFTCTSINGYPRPNVYWINKTDNSLLDQALQNDTVFLNMRGLYDVVSVLRIARTPSVNIGCC
IENVLLQQNLTVGSQTGNDIGERDKITENPVSTGEKAAATWSILAVLCLLVVVAVAIGWVCRDRCLQH
SYAGAWAVSPETELTGEFAVGSSRFWGAQGRLGCQLSFRVSKNFQKAKVPCLEQLLFLETQRSPRWCA
WHFLQPPLGMGWHPGVHFVTLRWDFPNMHRSRETSARPPRSPVPSPDQGVQGGSRHRRPAPMGCPEWV
QAPAPSPRGVSRAGPGTGAQPLWGVRSGSGHRQLLSVAATPAALVCPSVPGATLEG

| | | |
|---|---|---|
| NOV31d, 315925314 | SEQ ID NO: 775 | 871 bp |
| DNA Sequence | ORF Start: at 2 | ORF Stop: end of sequence |

CACCGGATCCGATACTCAGGAGAAGGAAGTCAGAGCGATGGTAGGCAGCGACGTGGAGCTCAGCTGCG
CTTGCCCTGAAGGAAGCCGTTTTGATTTAAATGATGTTTACGTATATTGGCAAACCAGTGAGTCGAAA
ACCGTGGTGACCTACCACATCCCACAGAACAGCTCCTTGGAAAACGTGGACAGCCGCTACCGGAACCG
AGCCCTGATGTCACCGGCCGGCATGCTGCGGGGCGACTTCTCCCTGCGCTTGTTCAACGTCACCCCCC
AGGACGAGCAGAAGTTTCACTGCCTGGTGTTGAGCCAATCCCTGGGATTCCAGGAGGTTTTGAGCGTT
GAGGTTACACTGCATGTGGCGGCAAACTTCAGCGTGCCCGTCGTCAGCGCCCCCCACAGCCCCTCCCA
GGATGAGCTCACCTTCACGTGTACATCCATAAACGGCTACCCCAGGCCCAACGTGTACTGGATCAATA
AGACGGACAACAGCCTGCTGGACCAGGCTCTGCAGAATGACACCGTCTTCTTGAACATGCGGGGCTTG
TATGACGTGGTCAGCGTGCTGAGGATCGCACGGACCCCCAGCGTGAACATTGGCTGCTGCATAGAGAA
CGTGCTTCTGCAGCAGAACCTGACTGTCGGCAGCCAGACAGGAAATGACATCGGAGAGAGAGACAAGA
TCACAGAGAATCCAGTCAGTACCGGCGAGAAAAACGCGGCCACGTGGAGCATCCTGGCTGTCCTGTGC
CTGCTTGTGGTCGTGGCGGTGGCCATAGGCTGGGTGTGCAGGGACCGATGCCTCCAACACAGCTATGC
AGGTGCCTGGGCTGTGAGTCCGGAGACAGAGCTCACTGGTCACCTGCTCGAGGGC

| | | | |
|---|---|---|---|
| NOV31d, 315925314 | | | |
| Protein Sequence | SEQ ID NO: 776 | 290 aa | MW at 31946.7kD |

TGSDTQEKEVRAMVGSDVELSCACPEGSRFDLNDVYVYWQTSESKTVVTYHIPQNSSLENVDSRYRNR
ALMSPAGMLRGDFSLRLFNVTPQDEQKFHCLVLSQSLGFQEVLSVEVTLHVAANFSVPVVSAPHSPSQ
DELTFTCTSINGYPRPNVYWINKTDNSLLDQALQNDTVFLNMRGLYDVVSVLRIARTPSVNIGCCIEN
VLLQQNLTVGSQTGNDIGERDKITENPVSTGEKNAATWSILAVLCLLVVVAVAIGWVCRDRCLQHSYA
GAWAVSPETELTGHLLEG

| | | |
|---|---|---|
| NOV31e, 315970230 | SEQ ID NO: 777 | 928 bp |
| DNA Sequence | ORF Start: at 2 | ORF Stop: end of sequence |

CACCGGATCCACCATGCGGCTGGGCAGTCCTGGACTGCTCTTCCTGCTCTTCAGCAGCCTTCGAGCTG
ATACTCAGGAGAAGGAAGTCAGAGCGATGGTAGGCAGCGACGTGGAGCTCAGCTGCGCTTGCCCTGAA
GGAAGCCGTTTTGATTTAAATGATGTTTACGTATATTGGCAAACCAGTGAGTCGAAAACCGTGGTGAC
CTACCACATCCCACAGAACAGCTCCTTGGAAAACGTGGACAGCCGCTACCGGAACCGAGCCCTGATGT
CACCGGCCGGCATGCTGCGGGGCGACTTCTCCCTGCGCTTGTTCAACGTCACCCCCCAGGACGAGCAG
AAGTTTCACTGCCTGGTGTTGAGCCAATCCCTGGGATTCCAGGAGGTTTTGAGCGTTGAGGTTACACT
GCATGTGGCGGCAAACTTCAGCGTGCCCGTCGTCAGCGCCCCCCACAGCCCCTCCCAGGATGAGCTCA
CCTTCACGTGTACATCCATAAACGGCTACCCCAGGCCCAACGTGTACCGGATCAATAAGACGGACAAC
AGCCTGCTGGACCAGGCTCTGCAGAATGACACCGTCTTCTTGAACATGCGGGGCTTGTATGACGTGGT
CAGCGTGCTGAGGATCGCACGGACCCCCAGCGTGAACATTGGCTGCTGCATAGAGAACGTGCTTCTGC
AGCAGAACCTGACTGTCGGCAGCCAGACAGGAAATGACATCGGAGAGAGAGACAAGATCACAGAGAAT
CCAGTCAGTACCGGCGAGAAAAACGCGGCCACGTGGAGCATCCTGGCTGTCCTGTGCCTGCTTGTGGT
CGTGGCGGTGGCCATAGGCTGGGTGTGCAGGGACCGATGCCTCCAACACAGCTATGCAGGTGCCTGGG
CTGTGAGTCCGGAGACAGAGCTCACTGGTCACCTGCTCGAGGGC

| | | | |
|---|---|---|---|
| NOV31e, 315970230 | | | |
| Protein Sequence | SEQ ID NO: 778 | 309 aa | MW at 33978.1kD |

TGSTMRLGSPGLLFLLFSSLEADTQEKEVRAMVGSDVELSCACPEGSRFDLNDVYVYWQTSESKTVVT
YHIPQNSSLENVDSRYRNRALMSPAGMLRGDFSLRLFNVTPQDEQKFHCLVLSQSLGFQEVLSVEVTL
HVAANFSVPVVSAPHSPSQDELTFTCTSINGYPRPNVYRINKTDNSLLDQALQNDTVFLNMRGLYDVV
SVLRIARTPSVNIGCCIENVLLQQNLTVGSQTGNDIGERDKITENPVSTGEKNAATWSILAVLCLLVV
VAVAIGWVCRDRCLQHSYAGAWAVSPETELTGHLLEG

| | | |
|---|---|---|
| NOV31f, CG55790-01 | SEQ ID NO: 779 | 912 bp |
| DNA Sequence | ORF Start: ATG at 4 | ORF Stop: TAA at 910 |

ACCATGCGGCTGGGCAGTCCTGGACTGCTCTTCCTGCTCTTCAGCAGCCTTCGAGCTGATACTCAGGA
GAAGGAAGTCAGAGCGATGGTAGGCAGCGACGTGGAGCTCAGCTGCGCTTGCCCTGAAGGAAGCCGTT
TTGATTTAAATGATGTTTACGTATATTGGCAAACCAGTGAGTCGAAAACCGTGGTGACCTACCACATC
CCACAGAACAGCTCCTTGGAAAACGTGGACAGCCGCTACCGGAACCGAGCCCTGATGTCACCGGCCGG
CATGCTGCGGGGCGACTTCTCCCTGCGCTTGTTCAACGTCACCCCCCAGGACGAGCAGAAGTTTCACT
GCCTGGTGTTGAGCCAATCCCTGGGATTCCAGGAGGTTTTGAGCGTTGAGGTTACACTGCATGTGGCA
GCAAACTTCAGCGTGCCCGTCGTCAGCGCCCCCCACAGCCCCTCCCAGGATGAGCTCACCTTCACGTG
TACATCCATAAACGGCTACCCCAGGCCCAACGTGTACTGGATCAATAAGACGGACAACAGCCTGCTGG
ACCAGGCTCTGCAGAATGACACCGTCTTCTTGAACATGCGGGGCTTGTATGACGTGGTCAGCGTGCTG
AGGATCGCACGGACCCCCAGCGTGAACATTGGCTGCTGCATAGAGAACGTGCTTCTGCAGCAGAACCT
GACTGTCGGCAGCCAGACAGGAAATGACATCGGAGAGAGAGACAAGATCACAGAGAATCCAGTCAGTA
CCGGCGAGAAAAACGCGGCCACGTGGAGCATCCTGGCTGTCCTGTGCCTGCTTGTGGTCGTGGCGGTG
GCCATAGGCTGGGTGTGCAGGGACCGATGCCTCCAACACAGCTATGCAGGTGCCTGGGCTGTGAGTCC
GGAGACAGAGCTCACTGGTCACCTGTAA

| | | | |
|---|---|---|---|
| NOV31f, CG55790-01 | | | |
| Protein Sequence | SEQ ID NO: 780 | 302 aa | MW at 33362.5kD |

MRLGSPGLLFLLFSSLRADTQEKEVRAMVGSDVELSCACPEGSRFDLNDVYVYWQTSESKTVVTYHIP
QNSSLENVDSRYRNRALMSPAGMLRGDFSLRLFNVTPQDEQKFHCLVLSQSLGFQEVLSVEVTLHVAA
NFSVPVVSAPHSPSQDELTFTCTSINGYPRPNVYWINKTDNSLLDQALQNDTVFLNNRGLYDVVSVLR
IARTPSVNIGCCIENVLLQQNLTVGSQTGNDIGERDKITENPVSTGEKNAATWSILAVLCLLVVVAVA
IGWVCPDRCLQHSYAGAWAVSPETELTGHL

| | | |
|---|---|---|
| NOV31g, 0G55790-03 | SEQ ID NO: 781 | 910 bp |
| DNA Sequence | ORF Start: ATG at 2 | ORF Stop: TAA at 908 |

CATGCGGCTGGGCAGTCCTGGACTGCTCTTCCTGCTCTTCAGCAGCCTTCGAGCTGATACTCAGGAGA
AGGAAGTCAGAGCGATGGTAGGCAGCGACGTGGAGCTCAGCTGCGCTTGCCCTGAAGGAAGCCGTTTT
GATTTAAATGATGTTTACGTATATTGGCAAACCAGTGAGTCGAAAACCGTGGTGACCTACCATATCCC
ACAGAACAGCTCCTTGGAAAACGTGGACAGCCGCTACCGGAACCGAGCCCTGATGTCACCGGCCGGCA
TGCTGCGGGGCGACTTCTCCCTGCGCTTGTTCAACGTCACCCCCCAGGACGAGCAGAAGTTTCACTGC
CTGGTGTTGAGCCAATCCCTGGGATTCCAGGAGGTTTTGAGCGTTGAGGTTACACTGCATGTGGCAGC
AAACTTCAGCGTGCCCGTCGTCAGCGCCCCCCACAGCCCCTCCCAGGATGAGCTCACCTTCACGTGTA
CATCCATAAACGGCTACCCCAGGCCCAACGTGTACTGGATCAATAAGACGGACAACAGCCTGCTGGAC
CAGGCTCTGCAGAATGACACCGTCTTCTTGAACATGCGGGGCTTGTATGACGTGGTCAGCGTGCTGAG
GATCGCACGGACCCCCAGCGTGAACATTGGCTGCTGCATAGAGAACGTGCTTCTGCAGCAGAACCTGA
GGCGAGAAAAACGCGGCCACGTGGAGCATCCTGGCTGTCCTGTGCCTGCTTGTGGTCGTGGCGGTGGC
CATAGGCTGGGTGTGCAGGGACCGATGCCTCCAACACAGCTATGCAGGTGCCTGGGCTGTGAGTCCGG
AGACAGAGCTCACTGGTCACCTGTAA

| | | | |
|---|---|---|---|
| NOV31g, CG55790-03 | | | |
| Protein Sequence | SEQ ID NO: 782 | 302 aa | MW at 33362.5kD |

MRLGSPGLLFLLFSSLRADTQEKEVRAMVGSDVELSCACPEGSRFDLNDVYVYWQTSESKTVVTYHIP
QNSSLENVDSRYRNEALMSPAGMLRGDFSLRLFNVTPQDEQKFHCLVLSQSLGFQEVLSVEVTLHVAA
NFSVPWSAPHSPSQDELTFTCTSINGYPRPNVYVVINKTDNSLLDQALQNDTVFLNMRGLYDVVSVLR
IARTPSVNIGCCIENVLLQQNLTVGSQTGNDIGERDKITENPVSTGEKNAATWSILAVLCLLVVVAVA
IGWVCRDRCLQHSYAGAWAVSPETELTGHL

| | | |
|---|---|---|
| NOV31h, CG55790-04 | SEQ ID NO: 783 | 705 bp |
| DNA Sequence | ORF Start: at 1 | ORF Stop: end of sequence |

GATACTCAGGAGAAGGAAGTCAGAGCGATGGTAGGCAGCGACGTGGAGCTCAGCTGCGCTTGCCCTGA
AGGAAGCCGTTTTGATTTAAATGATGTTTACGTATATTGGCAAACCAGTGAGTCGAAAACCGTGGTGA
CCTACCACATCCCACAGAACAGCTCCTTGGAAAACGTGGACAGCCGCTACCGGAACCGAGCCCTGATG
TCACCGGCCGGCATGCTGCGGGGCGACTTCTCCCTGCGCTTGTTCAACGTCACCCCCCAGGACGAGCA
GAAGTTTCACTGCCTGGTGTTGAGCCAATCCCTGGGATTCCAGGAGGTTTTGAGCGTTGAGGTTACAC
TGCATGTGGCAGCAAACTTCAGCGTGCCCGTCGTCAGCGCCCCCCACAGCCCCTCCCAGGATGAGCTC
ACCTTCACGTGTACATCCATAAACGGCTACCCCAGGCCCAACGTGTACTGGATCAATAAGACGGACAA
CAGCCTGCTGGACCAGGCTCTGCAGAATGACACCGTCTTCTTGAACATGCGGGGCTTGTATGACGTGG
TCAGCGTGCTGAGGATCGCACGGACCCCCAGCGTGAACATTGGCTGCTGCATAGAGAACGTGCTTCTG
CAGCAGAACCTGACTGTCGGCAGCCAGACAGGAAATGACATCGGAGAGAGAGACAAGATCACAGAGAA
TCCAGTCAGTACCGGCGAGAAAAAC

| | | | |
|---|---|---|---|
| NOV31h, CG55790-04 | | | |
| Protein Sequence | SEQ ID NO: 784 | 235 aa | MW at 26169.1kD |

DTQEKEVRAMVGSDVELSCACPEGSRFDLNDVYVYWQTSESKTVVTYHIPQNSSLENVDSRYRNRALM
SPAGMLRGDFSLRLFNVTPQDEQKFHCLVLSQSLGFQEVLSVEVTLHVAANFSVPVVSAPHSPSQDEL
TFTCTSINGYPRPNVYWINKTDNSLLDQALQNDTVFLNMRGLYDVVSVLRIARTPSVNIGCCIENVLL
QQNLTVGSQTGNDIGERDKITENPVSTGEKN

| | | |
|---|---|---|
| NOV31i, CG55790-05 | SEQ ID NO: 785 | 393 bp |
| DNA Sequence | ORF Start: at 11 | ORF Stop: at 1385 |

CACCAGATCTCTTCGAGCTGATACTCAGGAGAAGGAAGTCAGAGCGATGGTAGGCAGCGACGTGGAGC
TCAGCTGCGCTTGCCCTGAAGGAAGCCGTTTTGATTTAAATGATGTTTACGTATATTGGCAAACCAGT
GAGTCGAAAACCGTGGTGACCTACCACATCCCACAGAACAGCTCCTTGGAAAACGTGGACAGCCGCTA
CCGGAACCGAGCCCTGATGTCACCGGCCGGCATGCTGCGGGGCGACTTCTCCCTGCGCTTGTTCAACG
TCACCCCCCAGGACGAGCAGAAGTTTCACTGCCTGGTGTTGAGCCAATCCCTGGGATTCCAGGAGGTT
TTGAGCATTGAGGTTACACTGCATGTGGCAGCAAACTTCAGCGTGCCCGTCGTCAGCGCCCCCCACAG
CCCCTCCCAGGATGAGCTCACCTTCACGTGTACATCCATAAACGGCTACCCCAGGCCCAACGTGTACT
GGATCAATAAGACGGACAACAGCCTGCTGGACCAGGCTCTGCAGAATGACACCGTCTTCTTGAACATG
CGGGGCTTGTATGACGTGGTCAGCGTGCTGAGGATCGCACGGACCCCCAGCGTGAACATTGGCTGCTG
CATAGAGAACGTGCTTCTGCAGCAGAACCTGACTGTCGGCAGCCAGACAGGAAATGACATCGGAGAGA
GAGACAAGATCACAGAGAATCCAGTCAGTACCGGCGAGAAAAACGCGGCCACGTGGAGCATCCTGGCT
GTCCTGTGCCTGCTTGTGGTCGTGGCGGTGGCCATAGGCTGGGTGTGCAGGGACCGATGCCTCCAACA
CAGCTATGCAGGTGCCTGGGCTGTGAGTCCGGAGACAGAGCTCACTGGTGAGTTTGCCGTGGGAAGCA
GCAGGTTCTGGGGGGCCCAGGGGAGGCTTGGCTGCCAGCTGTCTTTCAGAGTTTCAAAAAACTTTCAA
AAGGCAAAAGTCCCTTGCCTTGAACAACTGTTGTTCCTGGAGACGCAGCGAAGCCCTCGATGGTGCGC
ATGGCATTTCCTGCAGCCTCCCCTTGGCATGGGATGGCATCCTGGTGTGCACTTTGTCACACTGCGAT
GGGATTTTCCCAACATGCACAGAAGCAGAGAGACGAGTGCTAGACCCCCGCGCTCCCCAGTGCCCAGC
CCCAACCAGGGTGTCCAGGGCGGGTCCAGGCACCGGCGCCCAGCCCCCATGGGGTGTCCGGAGTGGGT
CCAGGCACCGGCGCCCAGCCCCCGTGGGGTGTCCAGGGCGGGTCCAGGCACCGGCGCCCAGCCCCTGT
GGGGTGTCCGGAGCGGGTCCGGGCACCGCCAGCTTCTCTCTGTGGCAGCCACTCCTGCAGCTCTCGTT
TGCCCCTCAGTTCCAGGAGCAACACTCGAGGGC

| | | | |
|---|---|---|---|
| NOV31i, CG55790-05 | | | |
| Protein Sequence | SEQ ID NO: 786 | 458 aa | MW at 50317.7kD |

LRADTQEKEVRAMVGSDVELSCACPEGSRFDLNDVYVYWQTSESKTVVTYHIPQNSSLENVDSRYRNR
ALMSPAGMLRGDFSLRLFNVTPQDEQKFHCLVLSQSLGFQEVLSIEVTLHVAANFSVPVVSAPHSPSQ
DELTFTCTSINGYPRPNVYWINKTDNSLLDQALQNDTVFLNMRGLYDVVSVLRIARTPSVNIGCCIEN
VLLQQNLTVGSQTGNDIGERDKITENPVSTGEKNAATWSITAVLCLLVVVAVAIGWVCRDRCLQHSYA
GAWAVSPETELTGEFAVGSSRFWGAQGRLGCQLSFRVSKNFQKAKVPCLEQLLFLETQRSPRWCAWHF
LQPPLGMGWHPGVHFVTLRWDFPNNHRSRETSARPPRSPVPSPNQGVQGGSRHRRPAPMGCPEWVQAP
APSPRGVSRAGPGTGAQPLWGVRSGSGHRQLLSVAATPAALVCPSVPGAT

| | | |
|---|---|---|
| NOV31j, CG55790-06 | SEQ ID NO: 787 | 928 bp |
| DNA Sequence | ORF Start: ATG at 14 | ORF Stop: at 920 |

CACCGGATCCACCATGCGGCTGGGCAGTCCTGGACTGCTCTTCCTGCTCTTCAGCAGCCTTCGAGCTG
ATACTCAGGAGAAGGAAGTCAGAGCGATGGTAGGCAGCGACGTGGAGCTCAGCTGCGCTTGCCCTGAA
GGAAGCCGTTTTGATTTAAATGATGTTTACGTATATTGGCAAACCAGTGAGTCGAAAACCGTGGTGAC
CTACCACATCCCACAGAACAGCTCCTTGGAAAACGTGGACAGCCGCTACCGGAACCGAGCCCTGATGT
AAGTTTCACTGCCTGGTGTTGAGCCAATCCCTGGGATTCCAGGAGGTTTTGAGCATTGAGGTTACACT
CCTTCACGTGTACATCCATAAACGGCTACCCCAGGCCCAACGTGTACTGGATCAATAAGACGGACAAC
CACGTGCTGAGGAATCGCACGGACCCCCAGCGTGAACATTGGCTGCTGCATAGAGAACGTGCTTCTGC
AGCAGAACCTGACTGTCGGCAGCCAGACAGGAAATGACATCGGAGAGAGAGACAAGATCACAGAGAAT
CCAGTCAGTACCGGCGAGAAAAACGCGGCCACGTGGAGCATCCTGGCTGTCCTGTGCCTGCTTGTGGT
CGTGGCGGTGGCCATAGGCTGGGTGTGCAGGGACCGATGCCTCCAACACAGCTATGCAGGTGCCTGGG
CTGTGAGTCCGGAGACAGAGCTCACTGGTCACCTGCTCGAGGGC

| | | | |
|---|---|---|---|
| NOV31j, CG55790-06 | | | |
| Protein Sequence | SEQ ID NO: 788 | 1302 aa | MW at 33376.5kD |

MRLGSPGLLFLLFSSLRADTQEKEVRAMVGSDVELSCACPEGSRFDLNDVYVYWQTSESKTVVTYHIP
QNSSLENVDSRYRNRALMSPAGMLRGDFSLRLFNVTPQDEQKFHCLVLSQSLGFQEVLSIEVTLHVAA
NFSVPVVSAPHSPSQDELTFTCTSINGYPRPNVYWINKTDNSLLDQALQNDTVFLNMRGLYDVVSVLR
IARTPSVNIGCCIENVLLQQNLTVGSQTGNDIGERDKITENPVSTGEKNAATWSILAVLCLLVVVAVA
IGWVCRDRCLQHSYAGAWAVSPETELTGHL

| | | |
|---|---|---|
| NOV31k, CG55790-07 | SEQ ID NO: 789 | 871 bp |
| DNA Sequence | ORF Start: at 11 | ORF Stop: at 863 |

CACCGGATCCGATACTCAGGAGAAGGAAGTCAGAGCGATGGTAGGCAGCGACGTGGAGCTCAGCTGCG
CTTGCCCTGAAGGAAGCCGTTTTGATTTAAATGATGTTTACGTATATTGGCAAACCAGTGAGTCGAAA
ACCGTGGTGACCTACCACATCCCACAGAACAGCTCCTTGGAAAACGTGGACAGCCGCTACCGGAACCG
AGCCCTGATGTCACCGGCCGGCATGCTGCGGGGCGACTTCTCCCTGCGCTTGTTCAACGTCACCCCCC
AGGACGAGCAGAAGTTTCACTGCCTGGTGTTGAGCCAATCCCTGGGATTCCAGGAGGTTTTGAGCGTT
GAGGTTACACTGCATGTGGCGGCAAACTTCAGCGTGCCCGTCGTCAGCGCCCCCCACAGCCCCTCCCA
GGATGAGCTCACCTTCACGTGTACATCCATAAACGGCTACCCCAGGCCCAACGTGTACTGGATCAATA
AGACGGACAACAGCCTGCTGGACCAGGCTCTGCAGAATGACACCGTCTTCTTGAACATGCGGGGCTTG
TATGACGTGGTCAGCGTGCTGAGGATCGCACGGACCCCCAGCGTGAACATTGGCTGCTGCATAGAGAA
CGTGCTTCTGCAGCAGAACCTGACTGTCGGCAGCCAGACAGGAAATGACATCGGAGAGAGAGACAAGA
TCACAGAGAATCCAGTCAGTACCGGCGAGAAAAACGCGGCCACGTGGAGCATCCTGGCTGTCCTGTGC
CTGCTTGTGGTCGTGGCGGTGGCCATAGGCTGGGTGTGCAGGGACCGATGCCTCCAACACAGCTATGC
AGGTGCCTGGGCTGTGAGTCCGGAGACAGAGCTCACTGGTCACCTGCTCGAGGGC

| | | | |
|---|---|---|---|
| NOV31k, CG55790-07 | | | |
| Protein Sequence | SEQ ID NO: 790 | 284 aa | MW at 31402.1kD |

DTQEKEVRAMVGSDVELSCACPEGSRFDLNDVYVYWQTSESKTVVTYHIPQNSSLENVDSRYRNPALM
SPAGMLRGDFSLRLFNVTPQDEQKFHCLVLSQSLGFQEVLSVEVTLHVAANFSVPVVSAPHSPSQDEL
TFTCTSINGYPRPNVYWINKTDNSLLDQALQNDTVFLNMRGLYDVVSVLRIARTPSVNIGCCIENVLL
AVSPETELTGHL

| | | |
|---|---|---|
| NOV31l, | | |
| SNP1 3374852 of | SEQ ID NO: 791 | 912 bp |
| CG55790-01, | ORF Start: ATG at 4 | ORF Stop: TAA at 910 |
| DNA Sequence | SNP Pos: 296 | SNP Change: T to C |

ACCATGCGGCTGGGCAGTCCTGGACTGCTCTTCCTGCTCTTCAGCAGCCTTCGAGCTGATACTCAGGA
GAAGGAAGTCAGAGCGATGGTAGGCAGCGACGTGGAGCTCAGCTGCGCTTGCCCTGAAGGAAGCCGTT
TTGATTTAAATGATGTTTACGTATATTGGCAAACCAGTGAGTCGAAAACCGTGGTGACCTACCACATC
CCACAGAACAGCTCCTTGGAAAACGTGGACAGCCGCTACCGGAACCGAGCCCTGATGTCACCGGCCGG
CATGCTGCGGGGCGACTTCTCCCAGCGCTTGTTCAACGTCACCCCCCAGGACGAGCAGAAGTTTCACT
GCCTGGTGTTGAGCCAATCCCTGGGATTCCAGGAGGTTTTGAGCGTTGAGGTTACACTGCATGTGGCA
GCAAACTTCAGCGTGCCCGTCGTCAGCGCCCCCCACAGCCCCTCCCAGGATGAGCTCACCTTCACGTG
ACCAGGCTCTGCAGAATGACACCGTCTTCTTGAACATGCGGGGCTTGTATGACGTGGTCAGCGTGCTG
AGGATCGCACGGACCCCCAGCGTGAACATTGGCTGCTGCATAGAGAACGTGCTTCTGCAGCAGAACCT
GACTGTCGGCAGCCAGACAGGAAATGACATCGGAGAGAGAGACAAGATCACAGAGAATCCAGTCAGTA
CCGGCGAGAAAAACGCGGCCACGTGGAGCATCCTGGCTGTCCTGTGCCTGCTTGTGGTCGTGGCGGTG
GCCATAGGCTGGGTGTGCAGGGACCGATGCCTCCAACACAGCTATGCAGGTGCCTGGGCTGTGAGTCC
GGAGACAGAGCTCACTGGTCACCTGTAA

| | | | |
|---|---|---|---|
| NOV31l, | | | |
| SNP13374852 of | | | |
| CG55790-01, | SEQ ID NO: 792 | | MW at 33346.5kD |
| Protein Sequence | SNP Pos: 98 | 302 aa | SNP Change: Leu to Pro |

MRLGSPGLLFLLFSSLRADTQEKEVRAMVGSDVELSCACPEGSRFDLNDVYVYWQTSESKTVVTYHIP
QNSSLENVDSRYRNRALMSPAGMLRGDFSPRLFNVTPQDEQKFHCLVLSQSLGFQEVLSVEVTLHVAA
NFSVPVVSAPHSPSQDELTFTCTSINGYPRPNVYWINKTDNSLLDQALQNDTVFLNDRGLYDVVSVLR
IARTPSVNIGCCIENVLLQQNLTVGSQTGNDIGERDKITENPVSTGEKNAATWSILAVLCLLVVVAVA
IGWVCRDRCLQHSYAGAWAVSPETELTGHL

A ClustalW comparison of the above protein sequences yields the following sequence alignment shown in Table 31B.

TABLE 31B

Comparison of the NOV31 protein sequences.

| | |
|---|---|
| NOV31a | ----MRLGSPGLLFLLFSSLRADTQEKEVRAMVGSDVELSCACPEGSRFDLNDVYVYWQT |
| NOV31b | ----------------TRSLPADTQEKEVRANVGSDVELSCACPEGSRFDLNDVYVYWQT |
| NOV31c | ----------------TRSLRADTQEKEVPAMVGSDVELSCACPEGSRFDLNDVYVYWQT |
| NOV31d | -------------------TGSDTQEKEVRAMVGSDVELSCACPEGSRFDLNDVYVYWQT |
| NOV31e | TGSTMRLGSPGLLFLLFSSLRADTQEKEVRAMVGSDVELSCACPEGSRFDLNDVYVYWQT |
| NOV31f | ----MRLGSPGLLFLLFSSLRADTQEKEVRAMVGSDVELSCACPEGSRFDLNDVYVYWQT |
| NOV31g | ----MRLGSPGLLFLLFSSLRADTQEKEVRANVGSDVELSCACPEGSRFDLNDVYVYWQT |
| NOV31h | ----------------------DTQEKEVRAMVGSDVELSCACPEGSRFDLNDVYVYWQT |
| NOV31i | -------------------LRADTQEKEVRAMVGSDVELSCACPEGSRFDLNDVYVYWQT |
| NOV31j | ----MRLGSPGLLFLLFSSLRADTQEKEVRAMVGSDVELSCACPEGSRFDLNDVYVYWQT |
| NOV3lk | ----------------------DTQEKEVRAMVGSDVELSCACPEGSRFDLNDVYVYWQT |
| NOV31a | SESKTVVTYHIPQNSSLENVDSRYRNRALMSPAGMLRGDFSLRLFNVTPQDEQKFHCLVL |
| NOV31b | SESKTVVTYHIPQNSSLENVDSRYPNRALMSPAGMLRGDFSLRLFNVTPQDEQKFHCLVL |
| NOV31c | SESKTVVTYHIPQNSSLENVDSRYPNRALMSPAGMLRGDFSLRLFNVTPQDEQKFHCLVL |
| NOV31d | SESKTVVTYHIPQNSSLENVDSRYRNRALMSPAGMLRGDFSLRLFNVTPQDEQKFHCLVL |
| NOV31e | SESKTVVTYHIPQNSSLENVDSRYRNRALMSPAGMLRGDFSLRLFNVTPQDEQKFHCLVL |
| NOV31f | SESKTVVTYHIPQNSSLENVDSRYRNRALMSPAGMLRGDFSLRLFNVTPQDEQKFHCLVL |
| NOV31g | SESKTVVTYHIPQNSSLENVDSRYRNRALMSPAGMLRGDFSLRLFNVTPQDEQKFHCLVL |
| NOV31h | SESKTVVTYHIPQNSSLENVDSRYRNRALMSPAGMLRGDFSLRLFNVTPQDEQKFHCLVL |
| NOV31i | SESKTVVTYHIPQNSSLENVDSRYRNRALMSPAGMLRGDFSLRLFNVTPQDEQKFHCLVL |
| NOV31j | SESKTVVTYHIPQNSSLENVDSRYRNRALMSPAGMLRGDFSLRLFNVTPQDEQKFHCLVL |
| NOV31k | SESKTVVTYHIPQNSSLENVDSRYRNRALMSPAGMLRGDFSLRLFNVTPQDEQKFHCLVL |
| NOV31a | SQSLGFQEVLSVEVTLHVAANFSVPVVSAPHSPSQDELTFTCTSINGYPRPNVYWINKTD |
| NOV31b | SQSLGFQEVLSIEVTLHVAANFSVPVVSAPHSPSQDELTFTCTSINGYPRPNVYWINKTD |
| NOV31c | SQSLGFQEVLSIEVTLHVAANFSVPVVSAPHSPSQDELTFTCTSINGYPRPNVYWINKTD |
| NOV31d | SQSLGFQEVLSVEVTLHVAANFSVPVVSAPHSPSQDELTFTCTSINGYPRPNVYWINKTD |
| NOV31e | SQSLGFQEVLSVEVTLHVAANFSVPVVSAPHSPSQDELTFTCTSINGYPRPNVYRINKTD |
| Nov31f | SQSLGFQEVLSVEVTLHVAANFSVPVVSAPHSPSQDELTFTCTSINGYPRPNVYWINKTD |
| NOV31g | SQSLGFQEVLSVEVTLHVAANFSVPVVSAPHSPSQDELTFTCTSINGYPRPNVYWINKTD |
| NOV31h | SQSLGFQEVLSVEVTLHVAANFSVPVVSAPHSPSQDELTFTCTSINGYPRPNVYWINKTD |
| NOV31i | SQSLGFQEVLSIEVTLHVAANFSVPVVSAPHSPSQDELTFTCTSINGYPRPNVYWINKTD |
| NOV31j | SQSLGFQEVLSIEVTLHVAANFSVPVVSAPHSPSQDELTFTCTSINGYPRPNVYWINKTD |
| NOV31k | SQSLGFQEVLSVEVTLHVAANFSVPVVSAPHSPSQDELTFTCTSINGYPRPNVYWINKTD |
| NOV31a | NSLLDQALQNDTVFLNMRGLYDVVSVLRIARTPSVNIGCCIENVLLQQNLTVGSQTGNDI |
| NOV31b | NSLLDQALQNDTVFLNMRGLYDVVSVLRIARTPSVNIGCCIENVLLQQNLTVGSQTGNDI |
| NOV31c | NSLLDQALQNDTVFLNMRGLYDVVSVLRIARTPSVNIGCCIENVLLQQNLTVGSQTGNDI |
| NOV31d | NSLLDQALQNDTVFLNNRGLYDVVSVLRIARTPSVNIGCCIENVLLQQNLTVGSQTGNDI |
| NOV31e | NSLLDQALQNDTVFLNMRGLYDVVSVLRIARTPSVNIGCCIENVLLQQNLTVGSQTGNDI |
| Nov31f | NSLLDQALQNDTVFLNMRGLYDVVSVLRIARTPSVNIGCCIENVLLQQNLTVGSQTGNDI |
| NOV31g | NSLLDQALQNDTVFLNMRGLYDVVSVLRIARTPSVNIGCCIENVLLQQNLTVGSQTGNDI |
| NOV31h | NSLLDQALQNDTVFLNMRGLYDVVSVLRIARTPSVNIGCCIENVLLQQNLTVGSQTGNDI |
| NOV31i | NSLLDQALQNDTVFLNMRGLYDVVSVLRIARTPSVNIGCCIENVLLQQNLTVGSQTGNDI |
| NOV31j | NSLLDQALQNDTVFLNMRGLYDVVSVLRIARTPSVNIGCCIENVLLQQNLTVGSQTGNDI |
| NOV31k | NSLLDQALQNDTVFLNNRGLYDVVSVLRIARTPSVNIGCCIENVLLQQNLTVGSQTGNDI |
| NOV31a | GERDKITENPVSTGEKNAATWSILAVLCLLVVVAVAIGWVCRDRCLQHSYAGAWAVSPET |
| NOV31b | GERDKITENPVSTGEKNAATWSILAVLCLLVVVAVAIGWVCRDRCLQHSYAGAWAVSPET |
| NOV31c | GERDKITENPVSTGEKNAATWSILAVLCLLVVVAVAIGWVCRDRCLQHSYAGAWAVSPET |
| NOV31d | GERDKITENPVSTGEKNAATWSILAVLCLLVVVAVAIGWVCRDRCLQHSYAGAWAVSPET |
| NOV31e | GERDKITENPVSTGEKNAATWSILAVLCLLVVVAVAIGWVCRDRCLQHSYAGAWAVSPET |
| NOV31f | GERDKITENPVSTGEKNAATWSILAVLCLLVVVAVAIGWVCRDRCLQHSYAGAWAVSPET |
| NOV31g | GERDKITENPVSTGEKNAATWSILAVLCLLVVVAVAIGWVCRDRCLQHSYAGAWAVSPET |
| NOV31h | GERDKITENPVSTGEKN------------------------------------------- |
| NOV31i | GERDKITENPVSTGEKNAATWSILAVLCLLVVVAVAIGWVCRDRCLQHSYAGAWAVSPET |
| NOV31j | GERDKITENPVSTGEKNAATWSILAVLCLLVVVAVAIGWVCRDRCLQHSYAGAWAVSPET |
| NOV31k | GERDKITENPVSTGEKNAATWSILAVLCLLVVVAVAIGWVCRDRCLQHSYAGAWAVSPET |
| NOV31a | ELTGEFAVGSSRFWGAQGRLGCQLSFRVSKNFQKAKVPCLEQLLFLETQRSPRWCAWHFL |
| NOV31b | ELTGEFAVGSSRFWGAQGRLGCQLSFRVSKNFQKAKVPCLEQLLFLETQRSPRWCAWHFL |
| NOV31c | ELTGEFAVGSSRFWGAQGRLGCQLSFRVSKNFQKAKVPCLEQLLFLETQRSPRWCAWHFL |
| NOV31d | ELTGHLLEG--------------------------------------------------- |
| NOV31e | ELTGHLLEG--------------------------------------------------- |
| NOV31f | ELTGHL------------------------------------------------------ |
| NOV31g | ELTGHL------------------------------------------------------ |
| NOV31h | ------------------------------------------------------------ |
| NOV31i | ELTGEFAVGSSRFWGAQGRLGCQLSFRVSKNFQKAKVPCLEQLLFLETQRSPRWCAWHFL |
| NOV31j | ELTGHL------------------------------------------------------ |
| NOV31k | ELTGHL------------------------------------------------------ |
| NOV31a | QPPLGMGWHPGVHFVTLRWDFPNMHRSRETSARPPRSPVPSPDQGVQGGSRHRRPAPMGC |
| NOV31b | QPPLGMGWHPGVHFVTLRWDFPNMHRSRETSARPPRSPVPSPNQGVQGGSRHRRPAPMGC |
| NOV31c | QPPLGMGWHPGVHFVTLRWDFPNMHRSRETSARPPRSPVPSPDQGVQGGSRHRRPAPMGC |
| NOV31d | ------------------------------------------------------------ |
| NOV31e | ------------------------------------------------------------ |
| NOV31f | ------------------------------------------------------------ |
| NOV31g | ------------------------------------------------------------ |
| NOV31h | ------------------------------------------------------------ |
| NOV31i | QPPLGMGWHPGVHFVTLRWDFPNNHRSRETSARPPRSPVPSPNQGVQGGSRHRRPAPMGC |
| NOV31j | ------------------------------------------------------------ |
| NOV31k | ------------------------------------------------------------ |
| NOV31a | PEWVQAPAPSPRGVSEAGPGTGAQPLWGVRSGSGHRQLLSVAATPAALVCPSVPGAT--- |
| NOV31b | PEWVQAPAPSPRGVSRAGPGTGAQPLWGVRSGSGHRQLLSVAATPAALVCPSVPGATLEG |
| NOV31c | PEWVQAPAPSPRGVSRAGPGTGAQPLWGVRSGSGHRQLLSVAATPAALVCPSVPGATLEG |
| NOV31d | ------------------------------------------------------------ |
| NOV31e | ------------------------------------------------------------ |
| NOV3lf | ------------------------------------------------------------ |
| NOV31g | ------------------------------------------------------------ |
| NOV31h | ------------------------------------------------------------ |
| NOV31i | PEWVQAPAPSPRGVSPAGPGTGAQPLWGVRSGSGHRQLLSVAATPAALVCPSVPGAT--- |
| NOV31j | ------------------------------------------------------------ |
| NOV31k | ------------------------------------------------------------ |
| NOV31a | (SEQ ID NO: 770) |
| NOV31b | (SEQ ID NO: 772) |
| NOV31c | (SEQ ID NO: 774) |
| NOV31d | (SEQ ID NO: 776) |
| NOV31e | (SEQ ID NO: 778) |
| NOV3lf | (SEQ ID NO: 780) |
| NOV3lg | (SEQ ID NO: 782) |
| NOV3lh | (SEQ ID NO: 784) |
| NOV31i | (SEQ ID NO: 786) |
| NOV31j | (SEQ ID NO: 788) |
| NOV31k | (SEQ ID NO: 790) |

Further analysis of the NOV31 a protein yielded the following properties shown in Table 31C.

TABLE 31C
Protein Sequence Properties NOV31a

| | |
|---|---|
| SignalP analysis: | Cleavage site between residues 19 and 20 |
| PSORT II analysis: | |

| PSG: | a new signal peptide prediction method | |
|---|---|---|
| | N-region: | length 2; pos. chg 1; neg. chg 0 |
| | H-region: | length 14; peak value 10.56 |
| | PSG score: | 6.16 |

| | |
|---|---|
| GvH: | von Heijne's method for signal seq. recognition |
| | GvH score (threshold: −2.1): 1.98 |
| | possible cleavage site: between 20 and 21 |

>>> Seems to have a cleavable signal peptide (1 to 20)

| ALOM: | Klein et al's method for TM region allocation | | |
|---|---|---|---|
| | Init position for calculation: 21 | | |
| | Tentative number of TMS(s) for the threshold 0.5: 2 | | |
| | Number of TMS(s) for threshold 0.5: 1 | | |
| | INTEGRAL | Likelihood = | Transmembrane 258-274 |
| | | −13.27 | |

| | |
|---|---|
| PERIPHERAL | Likelihood = 2.97 (at 127) |

| | |
|---|---|
| | ALOM score: −13.27 (number of TMSs: 1) |
| MTOP: | Prediction of membrane topology (Hartmann et al.) |
| | Center position for calculation: 10 |
| | Charge difference: −2.0 C(0.0)-N(2.0) |
| | N >= C: N-terminal side will be inside |

>>> membrane topology: type 1a (cytoplasmic tail 275 to 473)

| MITDISC: | discrimination of mitochondrial targeting seq | | | |
|---|---|---|---|---|
| | R content: | 2 | Hyd Moment(75): | 5.99 |
| | Hyd Moment(95): | 8.79 | G content: | 2 |
| | D/E content: | 1 | S/T content: | 3 |

| | |
|---|---|
| | Score: −2.92 |
| Gavel: | prediction of cleavage sites for mitochondrial preseq |
| | R-2 motif at 27 LRA\|DT |

NUCDISC: discrimination of nuclear localization signals

| | |
|---|---|
| | pat4: RHRR (3) at 407 |
| | pat7: none |
| | bipartite: none |
| | content of basic residues: 8.9% |
| | NLS Score: −0.29 |
| KDEL: | ER retention motif in the C-terminus: none |

ER Membrane Retention Signals:

XXRR-like motif in the N-terminus: RLGS

none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: found

LL at 339
LL at 454

checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 76.7 |
| COIL: | Lupas's algorithm to detect coiled-coil regions |
| | total: 0 residues |

Final Results (k = 9/23):

55.6%: endoplasmic reticulum
22.2%: Golgi
11.1%: plasma membrane
11.1%: extracellular, including cell wall

>> prediction for CG55790-02 is end (k = 9)

A search of the NOV31a protein against the Geneseq database, a proprietary database that contains sequences published in patents and patent publication, yielded several homologous proteins shown in Table 31D.

TABLE 31D
Geneseq Results for NOV31a

| | | NOV31a | Identities/ | | |
|---|---|---|---|---|---|
| | | Residues/ | Similarities for | | |
| Geneseq | Protein/Organism/Length | Match | the Matched | | Expect |
| Identifier | [Patent #, Date] | Residues | Region | | Value |
| ABG30731 | Human B7-H2 transcript 2 (B7-H2 V2) | 1 . . . 473 | 471/473 | (99%) | 0.0 |
| | polypeptide - Homo sapiens, 473 aa. | 1 . . . 473 | 473/473 | (99%) | |
| | [WO200253733-A2, 11-JUL-2002] | | | | |
| AAU00422 | Human B7-3 protein - Homo sapiens, | 1 . . . 473 | 471/516 | (91%) | 0.0 |
| | 516 aa. [WO200112658-A2, 22-FEB- | 1 . . . 516 | 471/516 | (91%) | |
| | 2001] | | | | |
| AAU99789 | Human B7 related protein-1 (B7RP1) | 1 . . . 300 | 300/300 | (100%) | e−174 |
| | #2 - Homo sapiens, 302 aa. | 1 . . . 300 | 300/300 | (100%) | |
| | [WO200244364-A2, 06-JUN-2002] | | | | |
| AAG67292 | Amino acid sequence of a human hB7- | 1 . . . 300 | 300/300 | (100%) | e−174 |
| | H2 polypeptide - Homo sapiens, 302 | 1 . . . 300 | 300/300 | (100%) | |
| | aa. [WO200164704-A1, 07-SEP-2001] | | | | |
| AAB08729 | Amino acid sequence of a human | 1 . . . 300 | 300/300 | (100%) | e−174 |
| | B7RP1 polypeptide - Homo sapiens, | 1 . . . 300 | 300/300 | (100%) | |
| | 302 aa. [WO200046240-A2, 10-AUG- | | | | |
| | 2000] | | | | |

In a BLAST search of public sequence databases, the NOV31 a protein was found to have homology to the proteins shown in the BLASTP data in Table 31E.

TABLE 31E
Public BLASTP Results for NOV31a

| | | NOV31a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| O75144 | ICOS ligand precursor (B7 homolog 2) | 1 . . . 300 | 300/300 | (100%) | e−174 |
| | (B7-H2) (B7-like protein GI50) (B7- | 1 . . . 300 | 300/300 | (100%) | |
| | related protein-1) (B7RP-1) - Homo | | | | |
| | sapiens (Human), 302 aa. | | | | |
| CAD57064 | Sequence 11 from Patent WO0244364 - | 1 . . . 288 | 288/288 | (100%) | e−166 |
| | Mus musculus (Mouse), 288 aa | 1 . . . 288 | 288/288 | (100%) | |
| | (fragment). | | | | |
| Q9JHJ8 | ICOS ligand precursor (B7 homolog 2) | 7 . . . 295 | 135/291 | (46%) | 2e−58 |
| | (B7-H2) (B7-like protein GI50) (B7- | 32 . . . 317 | 172/291 | (58%) | |
| | related protein-1) (B7RP-1) (LICOS) - | | | | |
| | Mus musculus (Mouse), 322 aa. | | | | |
| O42404 | CD80-like protein - Gallus gallus | 2 . . . 283 | 99/291 | (34%) | 6e−33 |
| | (Chicken), 296 aa. | 3 . . . 288 | 153/291 | (52%) | |
| Q9BXR1 | Costimulatory molecule - Homo | 2 . . . 277 | 89/292 | (30%) | 3e−21 |
| | sapiens (Human), 316 aa. | 4 . . . 270 | 131/292 | (44%) | |

PFam analysis predicts that the NOV31a protein contains the domains shown in the Table 31F.

TABLE 31F
Domain Analysis of NOV31a

| | | Identities/ | |
|---|---|---|---|
| | | Similarities | |
| Pfam | NOV31a | for the | Expect |
| Domain | Match Region | Matched Region | Value |
| ig | 30 . . . 115 | 16/87 (18%) | 0.011 |
| | | 60/87 (69%) | |

### Example 32

The NOV32 clone was analyzed, and the nucleotide and encoded polypeptide sequences are shown in Table 32A.

TABLE 32A

NOV32 Sequence Analysis

| | | |
|---|---|---|
| NOV32a, CG55906-04 | SEQ ID NO: 793 | 4119 bp |
| DNA Sequence | ORF Start: ATG at 1 | ORF Stop: TAG at 4117 |

ATGTCTGCTCCAGACGAAGGGAGACGGGATCCCCCCAAACCGAAGGGCAAGACCCTGGGCAGCTTCTT
TGGGTCCCTGCCTGGCTTCAGCTCTGCCCGGAACCTGGTGGCCAACGCACATAGCTCGGCGAGAGCCC
GGCCGGCCGCTGACCCCACAGGAGCGCCTGCTGCCGAGGCTGCCCAACCACAGGCTCAGGTGGCTGCC
CACCCAGAGCAGACGGCCCCATGGACGGAGAAGGAGCTGCAACCTTCGGAAAAGCAGATGGTGTCCGG
GGCCAAAGACCTGGTGTGTTCCAAGATGTCCAGGGCCAAGGATGCCGTGTCCTCCGGGGTGGCCAGCG
TGGTGGACGTGGCTAAGGGAGTGGTCCAGGGAGGCCTGGACACCACTCGGTCTGCACTTACGGGCACC
GGACACCTCGAAGGCTGTCCTCACCGGCACCAAGGACACGGTGTCCACTGGGCTCACGGGGGCAGTGA
ATGTGGCCAAAGGGACCGTACAGGCCGGTGTGGACACCACCAAGACTGTGCTGACCGGCACCAAAGAC
ACAGTGACTACTGGGGTCATGGGGGCAGTGAACTTGGCCAAAGGGACTGTCCAGACTGGCGTGGAAAC
CTCCAAGGCTGTGCTGACCGGCACCAAAGATGCTGTGTCCACTGGGCTCACAGGGGCAGTGAATGTGG
CCAGAGGAAGCATTCAGACCGGTGTGGACACCAGTAAGACTGTCCTAACAGGTACCAAGGACACCGTC
TGTAGTGGGGTGACTGGTGCCATGAATGTGGCCAAAGGAACCATCCAGACCGGCGTGGACACCAGTAA
GACTGTCCTAACAGGTACCAAGGACACCGTCTGTAGTGGGGTGACTGGTGCCATGAATGTGGCCAAAG
GAACCATCCAGACCGGCGTGGACACCAGTAAGACTGTCCTAACAGGTACCAAGGACACCGTCTGTAGT
GGGGTGACTGGTGCCATGAATGTGGCCAAAGGAACCATCCAGACCGGCGTGGACACCACCAAGACTGT
CCTAACTGGCACCAAGAACACTGTCTGCAGTGGGGTGACCGGTGCCGTGAACTTGGCCAAAGAGGCCA
TCCAGGGGGGCCTGGATACCACCAAGTCTATGGTCATGGGTACGAAAGACACGATGTCCACTGGGCTC
ACAGGGGCAGCGAATGTGGCCAAGGGGGCCATGCAAACTGGGCTGAACACAACCCAAAATATCGCAAC
AGGTACAAAGGACACCGTCTGCAGTGGGGTGACTGGTGCCATGAATTTGGCCAGAGGAACCATCCAGA
CAGGCGTGGACACCACCAAGATCGTTCTAACTGGTACCAAGGACACTGTCTGCAGTGGGGTCACCGGT
GCTGCGAATGTGGCCAAAGGGGCCGTCCAGGGCGGCCTGGACACTACAAAGTCTGTCCTGACTGGCAC
TAAAGATGCTGTGTCCACTGGGCTCACAGGGGCTGTGAACGTGGCCAAAGGGACCGTCCAGACCGGCG
TAGACACCACCAAGACTGTCCTAACCGGCACCAAGGACACCGTCTGCAGTGGGGTGACCAGTGCTGTG
AACGTGGCCAAAGGGGCCGTCCAGGGGGGCCTGGACACCACCAAGTCTGTGGTCATAGGTACAAAAGA
CACGATGTCCACTGGGCTCACGGGGGCAGCGAATGTGGCCAAGGGGGCTGTCCAGACAGGTGTAGACA
CAGCCAAGACCGTGCTGACCGGCACCAAGGACACAGTGACTACTGGGCTCGTGGGGGCAGTGAATGTC
CTACAGTGGGGTCACCAGTGCCGTGAACGTGGCCAAGGGGGCTGTGCAAACTGGGCTGAAAACGACCC
AAAATATCGCGACAGGTACAAAGAACACCTTTGGCAGTGGGGTGACCAGTGCTGTGAATGTGGCCAAA
GGGGCTGCCCAGACAGGTGTAGACACGGCCAAGACCGTGCTGACCGGCACCAAGGACACAGTCACTAC
TGGGCTCATGGGGGCAGTGAATGTCGCCAAAGGGACTGTCCAGACCAGTGTGGACACCACCAAGACTG
TCCTAACTGGTACCAAGGACACCGTCTGCAGTGGGGTGACCGGTGCTGCGAATGTGGCCAAAGGGGCC
ATCCAAGGGGGCCTGGACACTACAAAGTCTGTCCTGACTGGCACTAAAGATGCTGTGTCCACTGGGCT
CACAGGGGCTGTGAAGTTGGCCAAAGGGACTGTCCAGACCGGCATGGACACCACCAAGACTGTGTTAA
CTGGTACCAAGGATGCTGTGTGCAGTGGGGTGACCGGTGCTGCGAATGTGGCCAAGGGGGCCGTCCAG
ATGGGTGTAGACACGGCCAAGACCGTGCTGACCGGTACCAAGGACACTGTCTGCAGTGGGGTCACCGG
TGCTGCGAACGTGGCCAAGGGTGCTGTGCAAACTGGGCTGAAAACGACCCAAAATATCGCAACAGGTA
CAAAGAACACCCTTGGCAGTGGGGTGACCGGTGCTGCGAAAGTGGCCAAAGGGGCCGTCCAGGGGGGC
CTGGACACTACAAAGTCTGTCCTGACTGGCACTAAAGATGCCGTGTCCACTGGGCTCACAGGGGCTGT
GAACTTGGCCAAAGGGACTGTCCAGACCGGCGTGGACACCAGCAAGACTGTCCTGACCGGTACCAAGG
ACACCGTCTGCAGTGGAGTCACTGGTGCCGTAAATGTGGCCAAAGGGACCGTCCAGACAGGTGTGGAC
ACAGCCAAGACGGTGCTGAGTGGCGCTAAGGATGCAGTGACTACTGGAGTCACGGGGGCAGTGAATGT
GGCCAAAGGAACCGTGCAGACCGGCGTGGACGCCTCCAAGGCTGTGCTTATGGGTACCAAGGACACTG
TCTTCAGTGGGGTTACCGGTGCCATGAGCATGGCCAAAGGGGCCGTCCAGGGGGGCCTGGACACCACC
AAGACAGTGCTGACCGGAACCAAAGACGCAGTGTCCGCTGGGCTCATGGGGTCAGGGAACGTGGCGAC
AGGGGCCACCCACACTGGCCTCAGCACCTTCCAGAACTGGTTACCTAGTACCCCCGCCACCTCCTGGG
GTGGACTCACCAGTTCCAGGACCACAGACAATGGTGGGGAGCAGACTGCCCTGAGCCCCCAAGAGGCC
AGCCACTACCAAGGGCCTTGCGACTGACGTGGCGACGTTCACCCAAGGGGCCGCCCCAGGCAGGGAGG
ACACGGGGCTTTTGGCCACCACACACGGCCCCGAAGAAGCCCCACGCTTGGCAATGCTGCAGAATGAG
TTGGAGGGGCTGGGGGACATCTTCCACCCCATGAATGCGGAGGAGCAAGCTCAGCTGGCTGCCTCCCA
GCCTGGGCCAAAGGTGCTGTCGGCGGAACAGGGGAGCTACTTCGTTCGTTTAGGTGACCTGGGTCCCA
GCTTCCGCCAGCGGGCATTTGAACACGCGGTGAGCCACCTGCAGCACGGCCAGTTCCAAGCCAGGGAC
ACTCTGGCCCAGCTCCAGGACTGCTTCAGGCTGATTGAAAAGGCCCAGCAGGCTCCAGAAGGGCAGCC
ACGTCTGGACCAGGGCTCAGGTGCCAGTGCGGAGGACGCTGCTGTCCAGGAGGAGCGGGATGCCGGGG
TTCTGTCCAGGGTCTGCGGCCTTCTCCGGCAGCTGCACACGGCCTACAGTGGCCTGGTCTCCAGCCTC
CAGGGCCTGCCCGCCGAGCTCCAGCAGCCAGTGGGGCGGGCGCGGCACAGCCTCTGTGAGCTCTATGG
CATCGTGGCCTCAGCTGGCTCTGTAGAGGAGCTGCCCGCAGAGCGGCTGGTGCAGAGCCGCGAGGGTG
GCACCAGGCTTGGCAGGGGTTAGAGCAGCTGCTGGAGGGCCTACAGCACAATCCCCCGCTCAGCTGG
CTGGTAGGGCCCTTCGCCTTGCCCGCTGGCGGGCAGTAG

| | | | |
|---|---|---|---|
| NOV32a, CG55906-04 | | | |
| Protein Sequence | SEQ ID NO: 794 | 1372 aa | MW at 136019.4kD |

MSAPDEGRRDPPKPKGKTLGSFFGSLPGFSSARNLVANAHSSARARPAADPTGAPAAEAAQPQAQVAA
HPEQTAPWTEKELQPSEKQMVSGAKDLVCSKMSRAKDAVSSGVASVVDVAKGVVQGGLDTTRSALTGT
KEVVSSGVTGAMDMAKGAVQGGLDTSKAVLTGTKDTVSTGLTGAVNVAKGTVQAGVDTTKTVLTGTKD
TVTTGVMGAVNLAKGTVQTGVETSKAVLTGTKDAVSTGLTGAVNVARGSIQTGVDTSKTVLTGTKDTV
CSGVTGAMNVAKGTIQTGVDTSKTVLTGTKDTVCSGVTGAMNVAKGTIQTGVDTSKTVLTGTKDTVCS
GVTGAMNVAKGTIQTGVDTTKTVLTGTKNTVCSGVTGAVNLAKEAIQGGLDTTKSMVMGTKDTMSTGL
TGAANVAKGAMQTGLNTTQNIATGTKDTVCSGVTGAMNLARGTIQTGVDTTKIVLTGTKDTVCSGVTG
AANVAKGAVQGGLDTTKSVLTGTKDAVSTGLTGAVNVAKGTVQTGVDTTKTVLTGTKDTVCSGVTSAV
NVAKGAVQGGLDTTKSVVIGTKDTMSTGLTGAANVAKGAVQTGVDTAKTVLTGTKDTVTTGLVGAVNV
AKGTVQTGMDTTKTVLTGTKDTIYSGVTSAVNVAKGAVQTGLKTTQNIATGTKNTFGSGVTSAVNVAK
GAAQTGVDTAKTVLTGTKDTVTTGLMGAVNVAKGTVQTSVDTTKTTLTGTKDTVCSGVTGAANVAKGA
IQGGLDTTKSVLTGTKDAVSTGLTGAVKLAXGTVQTGMDTTKTVLTGTKDAVCSGVTGAANVAKGAVQ
MGVDTAKTVLTGTKDTVCSGVTGAANVAKGAVQTGLKTTQNIATGTKNTLGSGVTGAAKVAKGAVQGG
LDTTKSVLTGTKDAVSTGLTGAVNLAKGTVQTGVDTSKTVLTGTKDTVCSGVTGAVNVAKGTVQTGVD
TAKTVLSGAKDAVTTGVTGAVNVAKGTVQTGVDASKAVLMGTKDTVFSGVTGAMSMAKGAVQGGLDTT
KTVLTGTKDAVSAGLMGSGNVATGATHTGLSTFQNWLPSTPATSWGGLTSSRTTDNGGEQTALSPQEA
PFSGISTPPDVLSVGPEPAWEAAATTKGLATDVATFTQGAAPGREDTGLLATTHGPEEAPRLAMLQNE
LEGLGDIFHPMNAEEQAQLAASQPGPKVLSAEQGSYFVRLGDLGPSFRQPAFEHAVSHLQHGQFQARD
TLAQLQDCFRLIEKAQQAPEGQPRLDQGSGASAEDAAVQEERDAGVLSRVCGLLRQLHTAYSGLVSSL
QGLPAELQQPVGRARHSLCELYGIVASAGSVEELPAERLVQSREGVHQAWQGLEQLLEGLQHNPPLSW
LVGPFALPAGGQ

| | | |
|---|---|---|
| NOV32b, CG55906-05 | SEQ ID NO: 795 | 6600 bp |
| DNA Sequence | ORF Start: ATG at 23 | ORF Stop: TAG at 4337 |

TCCAGCTTCTCACGTTCTCACTATGTCTGCTCCAGACGAAGGGAGACGGGATCCCCCCAAACCGAAGG
GCAAGACCCTGGGCAGCTTCTTTGGGTCCCTGCCTGGCTTCAACTCTGCCCGGAACCTGGTGGCCAAC
GCACATAGCTCGGCGAGAGCCCGGCCGGCCGCTGACCCCACAGGAGCGCCTGCTGCCGAGGCTGCCCA
ACCACAGGCTCAGGTGGCTGCCCACCCAGAGCAGACGGCCCCATGGACGGAGAAGGAGCTGCAACCTT
CGGAAAAGCAGATGGTGTCCGGGGCCAAAGACCTGGTGTGTTCCAAGATGTCCAGGGCCAAGGATGCC
GTGTCCTCCGGGGTGGCCAGCGTGGTGGACGTGGCTAAGGGAGTGGTCCAGGGAGGCCTGGACACCAC
TCGGTCTGCACTTACGGGCACCAAGGAGGCGGTGTCCAGCGGGGTCACAGGGGCCATGGACATGGCTA
AGGGGGCCGTCCAAGGGGGTCTGGACACCTCGAAGGCTGTCCTCACCGGCACCAAGGACACGGTGTCC
ACTGGGCTCACGGGGGCAGTGAATGTGGCCAAAGGGACCGTACAGGCCGGTGTGGACACCACCAAGAC
TGTGCTGACCGGCACCAAAGACACAGTGACTACTGGGGTCATGGGGGCAGTGAACTTGGCCAAAGGGA
CTGTCCAGACTGGCGTGGAAACCTCCAAGGCTGTGCTGACCGGCACCAAAGATGCTGTGTCCACTGGG
CTCACAGGGGCAGTGAATGTGGCCAGAGGAAGCATTCAGACCGGTGTGGACACCAGTAAGACTGTCCT
AACAGGTACCAAGGACACCGTCTGTAGTGGGGTGACCAGTGCCATGAATGTGGCCAAAGGAACCATCC
AGACCGGCGTGGACACCAGTAAGACTGTCCTAACAGGTACCAAGGACACCGTCTGTAGTGGGGTGACT
GGTGCCATGAATGTGGCCAAAGGAACCATCCAGACCGGCGTGGACACCAGTAAGACTGTCCTAACAGG
TACCAAGGACACCGTCTGTAGTGGGGTGACTGGTGCCATGAATGTGGCCAAAGGAACCATCCAGACCG
GCGTGGACACCACCAAGACTGTCCTAACTGGCACCAAGAACACTGTCTGCAGTGGGGTGACCGGTGCC
GTGAACTTGGCCAAAGAGGCCATCCAGGGGGGCCTGGATACCACCAAGTCTATGGTCATGGGTACGAA
AGACACGATGTCCACTGGGCTCACGGGGGCAGCGAATGTGGCCAAGGGGGCCATGCAAACTGGGCTGA
ACACAACCCAAAATATCGCAACAGGTACAAAGGACACCGTCTGCAGTGGGGTGACTGGTGCCATGAAT
TTGGCCAGAGGAACCATCCAGACAGGCGTGGACACCACCAAGATCGTTCTAACTGGTACCAAGGACAC
TGTCTGCAGTGGGGTCACCGGTGCTGCGAATGTGGCCAAAGGGGCCGTCCAGGGGGGCCTGGACACTA
CAAAGTCTGTCCTGACTGGCACTAAAGATGCTGTGTCCACTGGGCTCACAGGGGCTGTGAACGTGGCC
AAAGGGACCGTCCAGACCGGCGTAGACACCACCAAGACTGTCCTAACCGGCACCAAGGACACCGTCTG
CAGTGGGGTGACCAGTGCTGTGAACGTGGCCAAAGGGGCCGTCCAGGGGGGCCTGGACACCACCAAGT
CTGTGGTCATAGGTACAAAAGACACGATGTCCACTGGGCTCACGGGGGCAGCGAATGTGGCCAAGGGG
GCTGTCCAGACAGGTGTAGACACAGCCAAGACCGTGCTGACCGGCACCAAGGACACAGTGACTACTGG
GCTCGTGGGGGCAGTGAATGTCGCCAAAGGGACCGTCCAGACAGGCATGGACACCACCAAAACTGTCC
TAACCGGTACCAAGGACACCATCTACAGTGGGGTCACCAGTGCCGTGAACGTGGCCAAGGGGGCTGTG
CAAACTGGGCTGAAAACGACCCAAAATATCGCGACAGGTACAAAGAACACCTTTGGCAGTGGGGTGAC
CGGTGCTGTGAATGTGGCCAAAGGGGCCGTCCAGACAGGTGTAGACACAGCCAAGACCGTGCTGACCG
GCACCAAGGACACAGTCACTACTGGGCTCATGGGGGCAGTGAATGTCGCCAAAGGGACTGTCCAGACC
AGTGTGGACACCACCAAGACTGTCCTAACTGGTACCAAGGACACCGTCTGCAGTGGGGTGACCGGTGC
TGCGAATGTGGCCAAGGGGGCCGTCCAGACGGGTGTAGACACGGCCAAGACCGTGCTGACCGGTACCA
AGGACACTGTCTGCAGTGGGGTCACCGGTGCTGTGAACGTGGCCAAGGGTGCTGTGCAAACTGGGCTG
AAAACGACCCAAAATATCGCAACAGGTACAAAGAACACCCTTGGCAGTGGGGTGACCGGTGCTGCGAA
TGTGGCCAAAGGGGCCGTCCAGGGGGGCCTGGACACTACAAAGTCTGTCCTGACTGGCACTAAAGATG
CTGTGTCCACTGGGCTCACAGGGGCTGTGAACTTGGCCAAAGGGACTGTCCAGACCGGCATGGACACC
ACCAAGACTGTGTTAACTGGTACCAAGGATGCTGTGTGCAGTGGGGTGACCGGTGCTGCGAATGTGGC
CAAGGGGGCCGTCCAGACGGGTGTAGACACGGCCAAGACCGTGCTGACCGGCACCAAGGACACAGTCA
CTACTGGGCTCATGGGGGCAGTGAATGTCGCCAAAGGGACCGTCCAGACCAGTGTGGACACCACCAAG
ACTGTCCTAACTGGTACCAAGGACACCGTCTGCAGTGGGGTGACCGGTGCTGCGAATGTGGCCAAGGG
GGCCGTCCAGGGGGGCCTGGACACTACAAAGTCTGTCCTGACTGGCACTAAAGACACCGTATCCACTG
GGCTCACAGGGGCTGTGAACTTGGCCAAAGGGACTGTCCAGACCGGCGTGGACACCAGCAAGACTGTC
CTGACCGGTACCAAGGACACCGTCTGCAGTGGAGTCACTGGTGCCGTAAATGTGGCCAAAGGCACCGT
CCAGACAGGTGTGGACACAGCCAAGACGGTGCTGAGTGGCGCTAAGGATGCAGTGACTACTGGAGTCA
CGGGGGCAGTGAATGTGGCCAAAGGAACCGTGCAGACCGGCGTGGACGCCTCCAAGGCTGTGCTTATG
GGTACCAAGGACACTGTCTTCAGTGGGGTTACCGGTGCCATGAGCATGGCCAAAGGGGCCGTCCAGGG
GGGCCTGGACACCACCAAGACAGTGCTGACCGGAACCAAW3ACGCAGTGTCCGCTGGGCTCATGGGGT
CAGGGAACGTGGCGACAGGGGCCACCCACACTGGCCTCAGCACCTTCCAGAACTGGTTACCTAGTACC
CCCGCCACCTCCTGGGGTGGACTCACCAGTTCCAGGACCACAGACAATGGTGGGGAGCAGACTGCCCT
GAGCCCCCAAGAGGCCCCGTTCTCTGGCATCTCCACGCCCCCGGATGTGCTCAGTGTAGGCCCGGAGC
CTGCCTGGGAAGCCGCAGCCACTACCAAGGGCCTTGCGACTGACGTGGCGACGTTCACCCAAGGGGCC
GCCCCAGGCAGGGAGGACACGGGGCTTTTGACCACCACACACGGCCCCGAAGAAGCCCCACGCTTGGC
AATGCTGCAGAATGAGTTGGAGGGGCTGGGGGACATCTTCCACCCCATGAATGCGGAGGAGCAAGCTC
AGCTGGCTGCCTCCCAGCCCGGGCCAAAGGTGCTGTCGGCGGAACAGGGGAGCTACTTCGTTCGTTTA
GGTGACCTGGGTCCCAGCTTCCGCCAGCGGGCATTTGAACACGCGGTGAGCCACCTGCAGCACGGCCA
GTTCCAAGCCAGGGACACTCTGGCCCAGCTCCAGGACTGCTTCAGGCTGATTGAAAAGGCCCAGCAGG
CTCCAGAAGGGCAGCCACGTCTGGACCAGGGCTCAGGTGCCAGTGCGGAGGACGCTGCTGTCCAGGAG
GAGCGGGATGCCGGGGTTCTGTCCAGGGTCTGCGGCCTTCTCCGGCAGCTGCACACGGCCTACAGTGG
CCTGGTCTCCAGCCTCCAGGGCCTGCCCGCCGAGCTCCAGCAGCCAGTGGGGCGGGCGCGGCACAGCC
TCTGTGAGCTCTATGGCATCGTGGCCTCAGCTGGCTCTGTAGAGGAGCTGCCCGCAGAGCGGCTGGTG
CAGAGCCGCGAGGGTGTGCACCAGGCTTGGCAGGGGTTAGAGCAGCTGCTGGAGGGCCTACAGCACAA
TCCCCCGCTCAGCTGGCTGGTAGGGCCCTTCGCCTTGCCCGCTGGCGGGCAGTAGCTGTAGGAGCCTG
CAGGCCCGGCGCGGGGTCGCCCTGCTCTGTCCAGGGAGGAGCTGCCTCAGAACTTTCTCCCCGCCCCC
AAACCTGGATCGGTTCCCTAAAGCCCTAGACCTTTGGGGCTGCAGCTGGCTGAGCGCCGAGGGGCTGC
GGAGGCAGTGACCTTCTTAACTGAGCCACCCCACGCCCTGCTCCGGGCCTGCCTGCATCTCCCACCTC
CTCCCCAGCGCTGCCTGCCCCTCTCGGAGCCTGGGGTCACTCAGACCACCAGCCAAGAGCCTTCCCTT
GAAGTCCCCAAGCAAGCACTGCAATTAGGAAAGAGAAAAAGCAGCGTGCCCAGCCTGGAAGGGGATCT
GTTTGCCCCGCTAGCAACCCTTTTATATCTAGCAGGGCTCTTCCAGTCCTGCAGCACGGGCCCCCAGC
TATCAGCGGTGCAGGCAGTGCTGTGGCATCCCAGGCTCCGGGCAGCTCCGTTCTCATGCTGAAAGTGG
GTCTCCGGCCTTAGCACACACACCTTGAGGGTCTTAAGAACCACATTCCCTCATAGTAGAAAGTACTA
GAAAAAGCGACACTGCCATCATCATCCCAAGGCAGGCTGCTACTGCCTTTGCTGACCCCCGGGGTGGC
CTCACGGTGGGGACAAAGCTGCCAGGAGCCACAGCAGCCACAGCTGGGGCTTTGCACCAGCCTGGCTT
GAGACTGAGCAGTTTGCAGGGGGTGGGGGGTGCAAAAAACAAGCAAACAGGCTGCTGCTGCCTCCAGC
TGCCCACCACAGGCCTGCCCCAGGCACCTGGGGCTCTGAGGCCCCTGGGGAGGCTGGGCCCAAAAGCT
GCCCCTGGAGACACAGACAAAGGACTTCCCCGCAGGGAACTGTGCCCTATGGAGGGATCAGACAGGAA
CTGGGAACAGCCACAGAGGCTGCGTGCCTATGGCACAGCCCTTCCTCCGCCGCACACTCCCCCTGAAT
CCTCAGGCCCACCCAAGCGCCGGGCTGCAGAGGAAGCGGGGCTGGGGAGGCTGCAGGCATCAGAGACA
CTGGTGGTGGCGGACCCGGCCGCCGGGCCCCGTGCTCTCAGGCTAGCCCAGGTCGTGGAGGCTGGCAG
GCTCAGGTCGGGTGTGAGACGTGCCGTGGCTGCGCTCAGTCCAGCGGGGAGGAGCCGTTCAGCCCAAC
CTCCCCAGGAAGCCATATCCCCACTCACCCGGTAAGAGAACCTTGTCGTCCCCTTTCCATGCTCTCCT
AGGACACGAGCCCAGGAACCCCAGACCCAGGGGGAGGAAGGGTGGAGGGGCCCCAGGGGTCACCATGT
GCACCAGGGGCCGTGAGGGGCCGGGGCATTCAGCTCAGCTCTGAACCGGGGAAGCTGGCACGGCAAGG
ACTGCCTCAGGTGACGGGCCGTGAGAGGGGACGGGTCAGGAGCCTTCCCAAGCCTTCTCCTCAGCCCG
ACACCCATGGCCATCGGAGGCTAGGATGCCAGACACAGCCATTTGCAGAAATCAGGCACAGTGACTGC
AGCTCACGTCCAGCCAACCAAGCATGGGGCCGCAGCTCAGGAAGTCCCTTCCCGCCACACCACAGCCT
AATTCTTACTGGGACGGAGGCAACTCGGCTACGCTGGGCAGGACGACAAACACGAGACGCCACTGTGG
AATGAGCAACTTCGGAGCACGGGGTGACTTGCTTGGGACCGTGCCCACGTGACAGCCCCTTATGCAGA
GGAGGAAAGAGAAGCCCCGAGTGGGAGGGGAACCTGTCCAAAGTCACACGGTGTGTGGGTGACACAGC
TGGGGTGAGTCGAGGCTGGCCCCTGAGGCCCATGCTCCCTGAACGCTGGAGACCACTGTCGGCTAGCC
GCGGCTCTCAGGGAAGGCCTGGTCTCCACCCTCCCAGCCTAGCCTCGCGGACCCTCGTCCTCCCCACA
TCGGACCTGCTCACCTGCCTGGACCCTGGGCTGCCAGATGCAGGAAGCATCAAACCCCCCAGCCTCGT
GGGTGCGGGGCAGGGCGCAGGCAGCACAGCTTAGATGCCCTGGTTTGTCCCTCTTGTCTCCTGGGAAG
AGCTTGCTCCCGCCCAGCTCTCCTGCCACTGGCCTTTCAGGGTTGGGCTGGGCCCAGAGTGCCTTTTA
GTCGCTTCTCACGGTGGCCTGATGGCTCAACCCAGTCCCAAACGGGCCCAGTGACACTGCCGACTGCG
CCCCAGCTCAGGCCCCCACTGCACCAGCAATGCTAGAAAACCAAGCCAATAAAAGTGATTTCTTTTTT
CATT

| | | | |
|---|---|---|---|
| NOV32b, CG55906-05 | | | |
| Protein Sequence | SEQ ID NO: 796 | 1438 aa | MW at 142443.5kD |

MSAPDEGRRDPPKPKGKTLGSFFGSLPGFNSARNLVANAHSSARARPAADPTGAPAAEAAQPQAQVAA
HPEQTAPWTEKELQPSEKQMVSGAKDLVCSKMSRAKDAVSSGVASVVDVAKGVVQGGLDTTRSALTGT
KEAVSSGVTGAMDMAKGAVQGGLDTSKAVLTGTKDTVSTGLTGAVNVAKGTVQAGVDTTKTVLTGTKD
TVTTGVMGAVNLAKGTVQTGVETSKAVLTGTKDAVSTGLTGAVNVARGSIQTGVDTSKTVLTGTKDTV
CSGVTSAMNVAKGTIQTGVDTSKTVLTGTKDTVCSGVTGAMNVAKGTIQTGVDTSKTVLTGTKDTVCS
GVTGAMNVAKGTIQTGVDTTKTVLTGTKNTVCSGVTGAVNLAKEAIQGGLDTTKSMVMGTKDTMSTGL
TGAANVAKGAMQTGLNTTQNIATGTKDTVCSGVTGAMNLARGTIQTGVDTTKIVLTGTKDTVCSGVTG
AANVAKGAVQGGLDTTKSVLTGTKDAVSTGLTGAVNVAKGTVQTGVDTTKTVLTGTKDTVCSGVTSAV
NVAKGAVQGGLDTTKSVVIGTKDTMSTGLTGAANVAKGAVQTGVDTAKTVLTGTKDTVTTGLVGAVNV
AKGTVQTGMDTTKTVLTGTKDTIYSGTSAVNVAKGAVQTGLKTTQNIATGTKNTFGSGVTGAAVNVAK
GAVQTGVDTAKTVLTGTKDTVTTGLMGAVNVAKGTVQTSVDTTKTVLTGTKDTVCSGVTGAANVAXGA
VQTGVDTAKTVLTGTKDTVCSGVTGAVVAKGAVQTGLKTTQNIATGTKNTLGSGVTGAAANVAKGAVQ
GGLDTTKSVLTGTKDAVSTGLTGAVNLAKGTVQTGMDTTKTVLTGTKDAVCSGVTGAANVAKGAVQTG
VDTAKTVLTGTKDTVTTGLMGAVNVAKGTVQTSVDTTKTVLTGTKDTVCSGVTGAANVAKGAVQGGLD
TTKSVLTGTKDTVSTGLTGAVNLAKGTVQTGDVTSKTVLTGTKDTVCSGVTGAVNVAKGTVQTGVDTA
KTVLSGAKDAVTTGVTGAVNVAKGTVQTGVDASKAVLMGTKDTVFSGVTGAMSMAKGAVQGGLDTTKT
VLTGTKDAVSAGLMGSGNVATGATHTGLSTFQNWLPSTPATSWGGLTSSRTTDNGGEQTALSPQEAPF
SGISTPPDVLSVGPEPAVVEAATTKGLATDVATFTQGAAPGREDTGLLTTTHGPEEAPRLAMLQNELE
GLDIFHPMNAEEQAQLAASQPGPKVLSAEQGSYFVRLGDLGPSFRQRAFEHAVSHLQHGAQFQARDTL
AQLQDCFRLIEKAQQAPEGQPRLDQGSGASAEDAAVQEERDAGVLSRVCGLLRQLHTAYSGLVSSLQG
LPAELQQPVGRARHSLCELYGIVASAGSVEELPAERLVQSREGVHQAWQGLEQLLEGLQHNPPLSWLV
GPFALPAGGQ

| | | |
|---|---|---|
| NOV32c, CG55906-01 | SEQ ID NO: 797 | 3839 bp |
| DNA Sequence | ORF Start: ATG at 131 | ORF Stop: TAG at 3806 |

GTGAGGCCAGGCCTGCAGGTGGGTGTCGGGCTGCTCAGGCTTTCAGTGGGGAGTGGGTCTGGGATGGG
AGGCTAGGGAACCCCCATTCACGCACCTTCTCTGCCCCCTTCCAGCTTCTCACGTTCTCACTATGTCT
GCTCCAGACGAAGGGAGACGGGATCCCCCCAAACCGAAGGGCAAGCCCCCCGCCCCCATGCAGACCCT
GGGCAGCTTCTTTGGGTCCCTGCCTGGCTTCAGCTCTGCCCGGAACCTGGTGGCCAACGCACATAGCT
CGGTCGGGGCCAAAGACCTGGTGTGTTCCAAGATGTCCAGGGCCAAGGATGCCGTGTCCTCCGGGGTG
GCCAGCGTGGTGGACGTGGCTAAGGGAGTGGTCCAGGGAGGCCTGGACACCACTCGGTCTGCACTTAC
GGGCACCAAGGAGGTGGTGTCCAGCGGGGTCACAGGGGCCATGGACATGGCTAAGGGGGCCGTCCAAG
GGGGTCTGGACACCTCGAAGGCTGTCCTCACCGGCACCAAGGACACGGTGTCCACTGGGCTCACGGGG
GCAGTGAATGTGGCCAAAGGGACCGTACAGGCCGGTGTGGACACCACCAAGACTGTGCTGACCGGCAC
CAAAGACACAGTGACTACTGGGGTCATGGGGGCAGTGAACTTGGCCAAAGGGACTGTCCAGACTGGCG
TGGAAACCTCCAAGGCTGTGCTGACCGGCACCAAAGATGCTGTGTCCACTGGGCTCACAGGGGCAGTG
AATGTGGCCAGAGGAAGCATTCAGACCGGTGTGGACACCAGTAAGACTGTCCTAACAGGTACCAAGGA
CACCGTCTGTAGTGGGGTGACTGGTGCCATGAATGTGGCCAAAGGAACCATCCAGACCGGCGTGGACA
CCAGTAAGACTGTCCTAACAGGTACCAAGGACACCGTCTGTAGTGGGGTGACTGGTGCCATGAATGTG
GCCAAAGGAACCATCCAGACCGGCGTGGACACCAGTAAGACTGTCCTAACAGGTACCAAGGACACCGT
CTGTAGTGGGGTGACTGGTGCCATGAATGTGGCCAAAGGAACCATCCAGACCGGCGTGGACACCACCA
AGACTGTCCTAACTGGCACCAAGAACACTGTCTGCAGTGGGGTGACCGGTGCCGTGAACTTGGCCAAA
GAGGCCATCCAGGGGGGCCTGGATACCACCAAGTCTATGGTCATGGGTACGAAAGACACGATGTCCAC
TGGGCTCACAGGGGCAGCGAATGTGGCCAAGGGGGCCATGCAAACTGGGCTGAACACAACCCAAAATA
TCGCAACAGGTACAAAGGACACCGTCTGCAGTGGGGTGACTGGTGCCATGAATTTGGCCAGAGGAACC
ATCCAGACAGGCGTGGACACCACCAAGATCGTTCTAACTGGTACCAAGGACACTGTCTGCAGTGGGGT
CACCGGTGCTGCGAATGTGGCCAAAGGGGCCGTCCAGGGCGGCCTGGACACTACAAAGTCTGTCCTGA
CTGGCACTAAAGATGCTGTGTCCACTGGGCTCACAGGGGCTGTGAACGTGGCCAAAGGGACCGTCCAG
ACCGGCGTAGACACCACCAAGACTGTCCTAACCGGCACCAAGGACACCGTCTGCAGTGGGGTGACCAG
TGCTGTGAACGTGGCCAAAGGGGCCGTCCAGGGGGGCCTGGACACCACCAAGTCTGTGGTCATAGGTA
CAAAAGACACGATGTCCACTGGGCTCACGGGGGCAGCGAATGTGGCCAAGGGGGCTGTCCAGACAGGT
GTAGACACAGCCAAGACCGTGCTGACCGGCACCAAGGACACAGTGACTACTGGGCTCGTGGGGGCAGT
GAATGTCGCCAAAGGGACCGTCCAGACAGGCATGGACACCACCAAAACTGTCCTAACCGGTACCAAGG
ACACCATCTACAGTGGGGTCACCAGTGCCGTGAACGTGGCCAAGGGGGCTGTGCAAACTGGGCTGAAA
ACGACCCAAAATATCGCGACAGGTACAAAGAACACCTTTGGCAGTGGGGTGACCAGTGCTGTGAATGT
GGCCAAAGGGGCTGCCCAGACAGGTGTAGACACGGCCAAGACCGTGCTGACCGGCACCAAGGACACAG
TCACTACTGGGCTCATGGGGGCAGTGAATGTCGCCAAAGGGACTGTCCAGACCAGTGTGGACACCACC
AAGACTGTCCTAACTGGTACCAAGGACACCGTCTGCAGTGGGGTGACCGGTGCTGCGAATGTGGCCAA
AGGGGCCATCCAAGGGGGCCTGGACACTACAAAGTCTGTCCTGACTGGCACTAAAGATGCTGTGTCCA
CTGGGCTCACAGGGGCTGTGAAGTTGGCCAAAGGGACTGTCCAGACCGGCATGGACACCACCAAGACT
GTGTTAACTGGTACCAAGGATGCTGTGTGCAGTGGGGTGACCGGTGCTGCGAATGTGGCCAAGGGGGC
CGTCCAGATGGGTGTAGACACGGCCAAGACCGTGCTGACCGGTACCAAGGACACTGTCTGCAGTGGGG
TCACCGGTGCTGCGAACGTGGCCAAGGGTGCTGTGCAAACTGGGCTGAAAACGACCCAAAATATCGCA
ACAGGTACAAAGAACACCCTTGGCAGTGGGGTGACCGGTGCTGCGAAAGTGGCCAAAGGGGCCGTCCA
GGGGGGCCTGGACACTACAAAGTCTGTCCTGACTGGCACTAAAGATGCCGTGTCCACTGGGCTCACAG
GGGCTGTGAACTTGGCCAAAGGGACTGTCCAGACCGGCGTGGACACCAGCAAGACTGTCCTGACCGGT
ACCAAGGACACCGTCTGCAGTGGAGTCACTGGTGCCGTAAATGTGGCCAAAGGGACCGTCCAGACAGG
TGTGGACACAGCCAAGACGGTGCTGAGTGGCGCTAAGGATGCAGTGACTACTGGAGTCACGGGGGCAG
TGAATGTGGCCAAAGGAACCGTGCAGACCGGCGTGGACGCCTCCAAGGCTGTGCTTATGGGTACCAAG
GACACTGTCTTCAGTGGGGTTACCGGTGCCATGAGCATGGCCAAAGGGGCCGTCCAGGGGGGCCTGGA
CACCACCAAGACAGTGCTGACCGGAACCAAAGACGCAGTGTCCGCTGGGCTCATGGGGTCAGGGAACG
TGGCGACAGGGGCCACCCACACTGGCCTCAGCACCTTCCAGAACTGGTTACCTAGTACCCCCGCCACC
TCCTGGGGTGGACTCACCAGTTCCAGGACCACAGCTCAGCTGGCTGCCTCCCAGCCTGGGCCAAAGGT
GCTGTCGGCGGAACAGGGGAGCTACTTCGTTCGTTTAGGTGACCTGGGTCCCAGCTTCCGCCAGCGGG
CATTTGAACACGCGGTGAGCCACCTGCAGCACGGCCAGTTCCAAGCCAGGGACACTCTGGCCCAGCTC
CAGGACTGCTTCAGGCTGATTGAAAAGGCCCAGCAGGCTCCAGAAGGGCAGCCACGTCTGGACCAGGG
CTCAGGTGCCAGTGCGGAGGACGCTGCTGTCCAGGAGAGGGTCTGCGGCCTTCTCCGGCAGCTGCACA
CGGCCTACAGTGGCCTGGTCTCCAGCCTCCAGGGCCTGCCCGCCGAGCTCCAGCAGCCAGTGGGGCGG
GCGCGGCACAGCCTCTGTGAGCTCTATGGCATCGTGGCCTCAGCTGGCTCTGTAGAGGAGCTGCCCGC
AGAGCGGCTGGTGCAGAGCCGCGAGGGTGTGCACCAGGCTTGGCAGGGGTTAGAGCAGCTGCTGGAGG
GCCTACAGCACAATCCCCCGCTCAGCTGGCTGGTAGGGCCCTTCGCCTTGCCCGCTGGCGGGCAGTAG
CTGTAGGAGCCTGCAGGCCCGGCGCGGGGTC

| | | | |
|---|---|---|---|
| NOV32c, CG55906-01 | | | |
| Protein Sequence | SEQ ID NO: 798 | 1225 aa | MW at 120831.1kD |

MSAPDEGRPDPPKPKGKPPAPMQTLGSFFGSLPGFSSARNLVANAHSSVGAKDLVCSKMSRAKDAVSS
GVASVVDVAKGVVQGGLDTTRSALTGTKEVVSSGVTGAMDMAKGAVQGGLDTSKAVLTGTKDTVSTGL
TGAVNVAKGTVQAGVDTTKTVLTGTKDTVTTGVMGAVNLAKGTVQTGVETSKAVLTGTKDAVSTGLTG
AVNVARGSIQTGVDTSKTVLTGTKDTVCSGVTGAMNVAKGTIQTGVDTSKTVLTGTKDTVCSGVTGAM
NVAKGTIQTGVDTSKTVLTGTKDTVCSGVTGAMNVAKGTIQTGVDTTKTVLTGTKNTVCSGVTGAVNL
AKEAIQGGLDTTKSMVMGTKDTMSTGLTGAANVAKGAMQTGLNTTQNIATGTKDTVCSGVTGAMNLAR
GTIQTGVDTTKIVLTGTKDTVCSGVTGAANVAKGAVQGGLDTTKSVLTGTKDAVSTGLTGAVNVAKGT
VQTGVDTTKTVLTGTKDTVCSGVTSAVNVAKGAVQGGLDTTKSVVIGTKDTMSTGLTGAANVAKGAVQ
TGVDTAKTVLTGTKDTVTTGLVGAVNVAKGTVQTGMDTTKTVLTGTKDTIYSGVTSAVNVAKGAVQTG
LKTTQNIATGTKNTFGSGVTSAVNVAKGAAQTGVDTAKTVLTGTKDTVTTGLMGAVNVAKGTVQTSVD
TTKTVLTGTKDTVCSGVTGAANVAKGAIQGGLDTTKSVLTGTKDAVSTGLTGAVKVAKGTVQTGMDTT
KTVLTGTKDAVCSGVTGAANVAKGAVQMGVDTAXTVLTGTKDTVCSGVTGAANVAKGAVQTGLKTTQN
IATGTKNTLGSGVTGAAKVAXGAVQGGLDTTKSVLTGTKDAVSTGLTGAVNLAKGTVQTGVDTSKTVL
TGTKDTVCSGVTGAVNVAKGTVQTGVDTAKTVLSGAKDAVTTGVTGAVNVAKGTVQTGVDASKAVLMG
TKDTVFSGVTGAMSMAKGAVQGGLDTTKTVLTGTKDAVSAGLMGSGNVATGATHTGLSTFQNWLPSTP
ATSWGGLTSSRTTAQLAASQPGPKVLSAEQGSYFVRRGDLGPSFRQRAFEHAVSHLQHGQFQARDTLA
QLQDCFRLIEKAQQAPEGQPRLDQGSGASAEDAAVQERVCGLLRQLHTAYSGLVSSLQGLPAELQQPV
GRARHSLCELYGIVASAGSVEELPAERLVQSREGVHQAWQGLEQLLEGLQHNPPLSWLVGPFALPAGG
Q

| | | |
|---|---|---|
| NOV32d, 277901794 | SEQ ID NO: 799 | 2247 bp |
| DNA Sequence | ORF Start: at 1 | ORF Stop: end of sequence |

AGATCTATCATGTCTGCTCCAGACGAAGGGAGACGGGATCCCCCCAAACCGAAGGGCAAGACCCTGGG
CAGCTTCTTTGGGTCCCTGCCTGGCTTCAGCTCTGCCCGGAACCTGGTGGCCAACGCACATAGCTCGG
GTGGCTGCCCACCCAGAGCAGACGGCCCCATGGACGGAGAAGGAGCTGCAACCTTCGGAAAAGATGGT
GTCCGGGGCCAAAGACCTGGTGTGTTCCAAGATGTCCAGGGCCAAGGATGCCGTGTCCTCCGGGGTGG
CCAGCGTGGTGGACGTGGCTACGGGAGTGGTCCAGGGAGGCCTGGACACCACTCGGTCTGCACTTACG
GGCACCAAGGAGGCGGTGTCCAGCGGGTTCACAGGGGCCATGGACATGGCTAAGGGGGCCGTCCAAGG
GGGTCTGGACACCTCGAAGGCTGTCCTCACCGGCACCAAGGACACGGTGTCCACTGGGCTCACGGGGG
CAGTGAATGTGGCCAAAGGGACCGTACAGGCCGGTGTGGACACCACCAAGACTGTGCTGACCGGCACC
AAAGACACAGTGACTACTGGGGTCATGGGGGCAGTGAACTTGGCCAAAGGGACTGTCCAGACTGGCGT
GGAAACCTCCAAGGCTGTGCTGACCGGCACCAAAGATGCTGTGTCCACTGGGCTCACAGGGGCAGTGA
ATGTGGCCAGAGGAAGCATTCAGACCGGTGTGGACACCAGTAAGACTGTCCTAACAGGTACCAAGGAC
ACCGTCTGTAGTGGGGTGACCAGTGCCATGAATGTGGCCAAAGGAACCATCCAGACCGGCGTGGACAC
CAGCAAGACTGTCCTGACCGGTACCTTGGACACCGTCTGCAGTGGAGTCACTGGTGCCGTAAATGTGG
CCAAAGGCACCGTCCAGACAGGTGTGGACACAGCCAAGACGGTGCTGAGTGGCGCTAAGGATGCAGTG
GGCTGTGCTTATGGGTACCAAGGACACTGTCTTCAGTGGGGTTACCGGTGCCATGAGCATGGCCAAAG
GGGCCGTCCAGGGGGGCCTGGACACCACCAAGACAGTGCTGACCGGAACCAAAGACGCAGTGTCCGCT
GGGCTCATGGGGTCAGGGAACGTGGCGACAGGGGCCACCCACACTGGCCTCAGCACCTTCCAGAACTG
GTTACCTAGTACCCCCGCCACCTCCTGGGGTGGACTCACCAGTTCCAGGACCACAGACAATGGTGGGG
AGCAGACTGCCCTGAGCCCCCAAGAGGCCCCGTTCTCTGGCATCTCCACGCCCCCGGATGTGCTCAGT
GTAGGCCCGGAGCCTGCCTGGGAAGCCGCAGCCACTACCAAGGGCCTTGCGACTGACGTGGCGACGTT
CACCCAAGGGGCCGCCCCAGGCAGGGAGGACACGGGGCTTTTGACCACCACACACGGCCCCGAAGAAG
CCCCACGCTTGGCAATGCTGCAGAATGAGTTGGAGGGGCTGGGGGACATCTTCCACCCCATGAATGCG
GAGGAGCAAGCTCAGCTGGCTGCCTCCCAGCCCGGGCCAAAGGTGCTGTCGGCGGAACAGGGGAGCTA
TGCAGCACGGCCAGTTCCAAGCCAGGGACACTCTGGCCCAGCTCCAGGACTGCTTCAGGCTGATTGAA
TGCTGTCCAGGAGGAGCGGGATGCCGGGGTTCTGTCCAGGGTCTGCGGCCTTCTCCGGCAGCTGCACA
CGGCCTACAGTGGCCTGGTCTCCAGCCTCCAGGGCCTGCCCGCCGAGCTCCAGCAGCCAGTGGGGCGG
GCGCGGCACAGCCTCTGTGAGCTCTATGGCATCGTGGCCTCAGCTGGCTCTGTAGAGGAGCTGCCCGC
AGAGCGGCTGGTGCAGAGCCGCGAGGGTGTGCACCAGGCTTGGCAGGGGTTAGAGCAGCTGCTGGAGG
GCCTACAGCACAATCCCCCGCTCAGCTGGCTGGTAGGGCCCTTCGCCTTGCCCGCTGGCGGGCAGCTC
GAG

| | | | |
|---|---|---|---|
| NOV32d, 277901794 | | | |
| Protein Sequence | SEQ ID NO: 800 | 749 aa | MW at 75993.5kD |

RSIMSAPDEGRRDPPKPKGKTLGSFFGSLPGFSSARNLVANAHSSARARPAADPTGAPAAEAAQPQAQ
VAAHPEQTAPWTEKELQPSEKMVSGAKDLVCSKMSRAKDAVSSGVASVVDVATGVVQGGLDTTRSALT
GTKEAVSSGFTGAMDMAKGAVQGGLDTSKAVLTGTKDTVSTGLTGAVNVAKGTVQAGVDTTKTVLTGT
KDTVTTGVMGAVNLAKGTVQTGVETSKAVLTGTKDAVSTGLTGAVNVARGSIQTGVDTSKTVLTGTKD
TVCSGVTSAMNVAKGTIQTGVDTSKTVLTGTKDTVCSGVTGAVNVAKGTVQTGVDTAKTVLSGAKDAV
GLMGSGNVATGATHTGLSTFQNWLPSTPATSWGGLTSSRTTDNGGEQTALSPQEAPFSGISTPPDVLS
VGPEPAWEAAATTKGLATDVATFTQGAAPGREDTGLLTTTHGPEEAPRLAMLQNELEGLGDIFHPNNA
EEQAQLAASQPGPKVLSAEQGSYFVRLGDLGPSFRQRAFEHAVSHLQHGQFQARDTLAQLQDCFRLIE
KAQQAPEGQPRLDQGSGASAEDAAVQEERDAGVLSRVCGLLRQLHTAYSGLVSSLQGLPAELQQPVGR
ARHSLCELYGIVASAGSVEELPAERLVQSREGVHQAWQGLEQLLEGLQHNPPLSWLVGPFALPAGGQL
E

| | | |
|---|---|---|
| NOV32e, CG55906-02 | SEQ ID NO: 801 | 810 bp |
| DNA Sequence | ORF Start: ATG at 123 | ORF Stop: TAG at 792 |

AGGCCTGCAGGTGGGTGTCGGGCTGCTCAGGCTTTCAGTGGGGAGTGGGTGTGGGATGGGAGGCTAGG
GAACCCCCATTCACGCACCTTCTCTGCCCCCTTCCAGCTTCTCACGTTCTCACTATGTCTGCTCCAGA
CGAAGGGAGACGGGATCCCCCCAAACCGAAGGGCAAGACCCTGGGCAGCTTCTTTGGGTCCCTGCCTG
GCTTCAACTCTGCCCGGAACCTGGTGGCCAACGCACATAGCTCGGCGAGAGCCCGGCCGGCCGCTGAC
CCCACAGGAGCGCCTGCTGCCGAGGCTGCCCAACCACAGGCTCAGGTGGCTGCCCACCCAGAGCAGAC
GGCCCCATGGACGGAGAAGGAGCTGCAACCTTCGGAAAAGATTGAAAAGGCCCAGCAGGCTCCAGAAG
GGCAGCCACGTCTGGACCAGGGCTCAGGTGCCAGTGCGGAGGACGCTGCTGTCCAGGAGGAGCGGGAT
GCCGGGGTTCTGTCCAGGGTCTGCGGCCTTCTCCGGCAGCTGCACACGGCCTACAGTGGCCTGGTCTC
CAGCCTCCGGGGCCTGCCCGCCGAGCTCCAGCAGCCAGTGGGGCGGGCGCGGCACAGCCTCTGTGAGC
TCTATGGCATCGTGGCCTCAGCTGGCTCTGTAGAGGAGCTGCCCGCAGAGCGGCTGGTGCAGAGCCGC
GAGGGTGTGCACCAGGCTTGGCAGGGGTTAGAGCAGCTGCTGGAGGGCCTACAGCACAATCCCCCGCT
CAGCTGGCTGGTAGGGCCCTTCGCCTTGCCCGCTGGCGGGCAGTAGCTGTAGGAGCCTGCAG

| | | | |
|---|---|---|---|
| NOV32e, CG55906-02 | | | |
| Protein Sequence | SEQ ID NO: 802 | 223 aa | MW at 23486.9kD |

MSAPDEGRRDPPKPKGKTLGSFFGSLPGFNSARNLVANAHSSARARPAADPTGAPAAEAAQPQAQVAA
HPEQTAPWTEKELQPSEKIEKAQQAPEGQPRLDQGSGASAEDAAVQEERDAGVLSRVCGLLRQLHTAY
SGLVSSLRGLPAELQQPVGRARHSLCELYGIVASAGSVEELPAERLVQSREGVHQAWQGLEQLLEGLQ
HNPPLSWLVGPFALPAGGQ

| | | |
|---|---|---|
| NOV32f, 230272941 | SEQ ID NO: 803 | 714 bp |
| DNA Sequence | ORF Start: at 2 | ORF Stop: TGA at 692 |

GGCGCGCCCACCCTTCACCACCATGTCTGCTCCAGACGAAGGGAGACGGGATCCCCCCAAACCGAAGG
GCAAGACCCTGGGCAGCTTCTTTGGGTCCCTGCCTGGCTTCAGCTCTGCCCGGAACCTGGTGGCCAAC
GCACATAGCTCGGCGAGAGCCCGGCCGGCCGCTGACCCCACAGGAGCGCCTGCTGCCGAGGCTGCCCA
ACCACAGGCTCAGGTGGCTGCCCACCCAGAGCAGACGGCCCCATGGACGGAGAAGGAGCTGCAACCTT
CGGAAAAGATTGAAAAGGCCCAGCAGGCTCCAGAAGGGCAGCCACGTCTGGACCAGGGCTCAGGTGCC
AGTGCGGAGGACGCTGCTGTCCAGGAGGAGCGGGATGCCGGGGTTCTGTCCAGGGTCTGCGGCCTTCT
CCGGCAGCTGCACACGGCCTACAGTGGCCTGGTCTCCAGCCTCCAGGGCCTGCCCGCCGAGCTCCAGC
AGCCAGTGGGGCGGGCGCGGCACAGCCTCTGTGAGCTCTATGGCATCGTGGCCTCAGCTGGCTCTGTA
GAGGAGCTGCCCGCAGAGCGGCTGGTGCAGAGCCGCGAGGGTGTGCACCAGGCTTGGCAGGGGTTAGA
GCAGCTGCTGGAGGGCCTACAGCACAATCCCCCGCTCAGCTGGCTGGTAGGGCCCTTCGCCTTGCCCG
CTGGCGGGCAGTGAAGGGGGCGGCCGCGGAGCCT

| | | | |
|---|---|---|---|
| NOV32f, 230272941 | | | |
| Protein Sequence | SEQ ID NO: 804 | 230 aa | MW at 24202.7kD |

ARPPFTTMSAPDEGRRDPPKPKGKTLGSFFGSLPGFSSARNLVANAHSSAPARPAADPTGAPAAEAAQ
PQAQVAAHPEQTAPWTEKELQPSEKIEKAQQAPEGQPRLDQGSGASAEDAAVQEERDAGVLSRVCGLL
RQLHTAYSGLVSSLQGLPAELQQPVGRARHSLCELYGIVASAGSVEELPAERLVQSREGVHQAWQGLE
QLLEGLQHNPPLSWLVGPFALPAGGQ

| | | |
|---|---|---|
| NOV32g, CG55906-03 | SEQ ID NO: 805 | 714 bp |
| DNA Sequence | ORF Start: ATG at 23 | ORF Stop: TGA at 692 |

GGCGCGCCCACCCTTCACCACCATGTCTGCTCCAGACGAAGGGAGACGGGATCCCCCCAAACCGAAGG
GCAAGACCCTGGGCAGCTTCTTTGGGTCCCTGCCTGGCTTCAGCTCTGCCCGGAACCTGGTGGCCAAC
GCACATAGCTCGGCGAGAGCCCGGCCGGCCGCTGACCCCACAGGAGCGCCTGCTGCCGAGGCTGCCCA
ACCACAGGCTCAGGTGGCTGCCCACCCAGAGCAGACGGCCCCATGGACGGAGAAGGAGCTGCAACCTT
CGGAAAAGATTGAAAAGGCCCAGCAGGCTCCAGAAGGGCAGCCACGTCTGGACCAGGGCTCAGGTGCC
AGTGCGGAGGACGCTGCTGTCCAGGAGGAGCGGGATGCCGGGGTTCTGTCCAGGGTCTGCGGCCTTCT
CCGGCAGCTGCACACGGCCTACAGTGGCCTGGTCTCCAGCCTCCAGGGCCTGCCCGCCGAGCTCCAGC
AGCCAGTGGGGCGGGCGCGGCACAGCCTCTGTGAGCTCTATGGCATCGTGGCCTCAGCTGGCTCTGTA
GAGGAGCTGCCCGCAGAGCGGCTGGTGCAGAGCCGCGAGGGTGTGCACCAGGCTTGGCAGGGGTTAGA
GCAGCTGCTGGAGGGCCTACAGCACAATCCCCCGCTCAGCTGGCTGGTAGGGCCCTTCGCCTTGCCCG
CTGGCGGGCAGTGAAGGGGGCGGCCGCGGAGCCT

| | | | |
|---|---|---|---|
| NOV32g, CG55906-03 | | | |
| Sequence | SEQ ID NO: 806 | 223 aa | MW at 23431.8kD |

MSAPDEGRRDPPKPKGKTLGSFFGSLPGFSSARNLVANAHSSARARPAADPTGAPAAEAAQPQAQVAA
HPEQTAPWTEKELQPSEKIEKAQQAPEGQPRLDQGSGASAEDAAVQEERDAGVLSRVCGLLRQLHTAY
SGLVSSLQGLPAELQQPVGRARHSLCELYGIVASAGSVEELPAERLVQSREGVHQAWQGLEQLLEGLQ
HNPPLSWLVGPFALPAGGQ

| | | |
|---|---|---|
| NOV32h, | | |
| SNP13382505 of | SEQ ID NO: 807 | 3839 bp |
| CG55906-01, | ORF Start: ATG at 131 | ORF Stop: TAG at 3806 |
| DNA Sequence | SNP Pos: 3393 | SNP Change: C to T |

GTGAGGCCAGGCCTGCAGGTGGGTGTCGGGCTGCTCAGGCTTTCAGTGGGGAGTGGGTGTGGGATGGC
AGGCTAGGGAACCCCCATTCACGCACCTTCTCTGCCCCCTTCCAGCTTCTCACGTTCTCACTATGTCT
GCTCCAGACGAAGGGAGACGGGATCCCCCCAAACCGAAGGGCAAGCCCCCCGCCCCCATGCAGACCCT
GGGCAGCTTCTTTGGGTCCCTGCCTGGCTTCAGCTCTGCCCGGAACCTGGTGGCCAACGCACATAGCT
CGGTCGGGGCCAAAGACCTGGTGTGTTCCAAGATGTCCAGGGCCAAGGATGCCGTGTCCTCCGGGGTG
GCCAGCGTGGTGGACGTGGCTAAGGGAGTGGTCCAGGGAGGCCTGGACACCACTCGGTCTGCACTTAC
GGGCACCAAGGAGGTGGTGTCCAGCGGGGTCACAGGGGCCATGGACATGGCTAAGGGGGCCGTCCAAG
GGGGTCTGGACACCTCGAAGGCTGTCCTCACCGGCACCAAGGACACGGTGTCCACTGGGCTCACGGGG
GCAGTGAATGTGGCCAAAGGGACCGTACAGGCCGGTGTGGACACCACCAAGACTGTGCTGACCGGCAC
CAAAGACACAGTGACTACTGGGGTCATGGGGGCAGTGAACTTGGCCAAAGGGACTGTCCAGACTGGCG
TGGAAACCTCCAAGGCTGTGCTGACCGGCACCAAAGATGCTGTGTCCACTGGGCTCACAGGGGCAGTG
AATGTGGCCAGAGGAAGCATTCAGACCGGTGTGGACACCAGTAAGACTGTCCTAACAGGTACCAAGGA
CACCGTCTGTAGTGGGGTGACTGGTGCCATGAATGTGGCCAAAGGAACCATCCAGACCGGCGTGGACA
CCAGTAAGACTGTCCTAACAGGTACCAAGGACACCGTCTGTAGTGGGGTGACTGGTGCCATGAATGTG
GCCAAAGGAACCATCCAGACCGGCGTGGACACCAGTAAGACTGTCCTAACAGGTACCAAGGACACCGT
CTGTAGTGGGGTGACTGGTGCCATGAATGTGGCCAAAGGAACCATCCAGACCGGCGTGGACACCACCA
AGACTGTCCTAACTGGCACCAAGAACACTGTCTGCAGTGGGGTGACCGGTGCCGTGAACTTGGCCAAA
GAGGCCATCCAGGGGGGCCTGGATACCACCAAGTCTATGGTCATGGGTACGAAAGACACGATGTCCAC
GGGACTCACAGGGGCAGCGAATGTGGCCAAGGGGGCCATGCAAACTGGGCTGAACACAACCCAAAATA
TCGCAACAGGTACAAAGGACACCGTCTGCAGTGGGGTGACTGGTGCCATGAATTTGGCCAGAGGAACC
ATCCAGACAGGCGTGGACACCACCAAGATCGTTCTAACTGGTACCAAGGACACTGTCTGCAGTGGGGT
CACCGGTGCTGCGAATGTGGCCAAAGGGGCCGTCCAGGGCGGCCTGGACACTACAAAGTCTGTCCTGA
CTGGCACTAAAGATGCTGTGTCCACTGGGCTCACAGGGGCTGTGAACGTGGCCAAAGGGACCGTCCAG
ACCGGCGTAGACACCACCAAGACTGTCCTAACCGGCACCAAGGACACCGTCTGCAGTGGGGTGACCAG
TGCTGTGAACGTGGCCAAAGGGGCCGTCCAGGGGGGCCTGGACACCACCAAGTCTGTGGTCATAGGTA
CAAAAGACACGATGTCCACTGGGCTCACGGGGGCAGCGAATGTGGCCAAGGGGGCTGTCCAGACAGGT
GTAGACACAGCCAAGACCGTGCTGACCGGCACCAAGGACACAGTGACTACTGGGCTCGTGGGGGCAGT
GAATGTCGCCAAAGGGACCGTCCAGACAGGCATGGACACCACCAAAACTGTCCTAACCGGTACCAAGG
ACGACCCAAAATATCGCGACAGGTACAAAGAACACCTTTGGCAGTGGGGTGACCAGTGCTGTGAATGT
GGCCAAAGGGGCTGCCCAGACAGGTGTAGACACGGCCAAGACCGTGCTGACCGGCACCAAGGACACAG
TCACTACTGGGCTCATGGGGGCAGTGAATGTCGCCAAAGGGACTGTCCAGACCAGTGTGGACACCACC
AAGACTGTCCTAACTGGTACCAAGGACACCGTCTGCAGTGGGGTGACCGGTGCTGCGAATGTGGCCAA
AGGGGCCATCCAAGGGGGCCTGGACACTACAAAGTCTGTCCTGACTGGCACTAAAGATGCTGTGTCCA
CTGGGCTCACAGGGGCTGTGAAGTTGGCCAAAGGGACTGTCCAGACCGGCATGGACACCACCAAGACT
GTGTTAACTGGTACCAAGGATGCTGTGTGCAGTGGGGTGACCGGTGCTGCGAATGTGGCCAAGGGGGC
CGTCCAGATGGGTGTAGACACGGCCAAGACCGTGCTGACCGGTACCAAGGACACTGTCTGCAGTGGGG
TCACCGGTGCTGCGAACGTGGCCAAGGGTGCTGTGCAAACTGGGCTGAAAACGACCCAAAATATCGCA
ACAGGTACAAAGAACACCCTTGGCAGTGGGGTGACCGGTGCTGCGAAAGTGGCCAAAGGGGCCGTCCA
GGGGGGCCTGGACACTACAAAGTCTGTCCTGACTGGCACTAAAGATGCCGTGTCCACTGGGCTCACAG
GGGCTGTGAACTTGGCCAAAGGGACTGTCCAGACCGGCGTGGACACCAGCAAGACTGTCCTGACCGGT
ACCAAGGACACCGTCTGCAGTGGAGTCACTGGTGCCGTAAATGTGGCCAAAGGGACCGTCCAGACAGG
TGTGGACACAGCCAAGACGGTGCTGAGTGGCGCTAAGGATGCAGTGACTACTGGAGTCACGGGGGCAG
TGAATGTGGCCAAAGGAACCGTGCAGACCGGCGTGGACGCCTCCAAGGCTGTGCTTATGGGTACCAAG
GACACTGTCTTCAGTGGGGTTACCGGTGCCATGAGCATGGCCAAAGGGGCCGTCCAGGGGGGCCTGGA
CACCACC)AGACAGTGCTGACCGGAACCAAAGACGCAGTGTCCGCTGGGCTCATGGGGTCAGGGAACG
TGGCGACAGGGGCCACCCACACTGGCCTCAGCACCTTCCAGAACTGGTTACCTAGTACCCCCGCCACC
TCCTGGGGTGGACTCACCAGTTCCAGGACCACAGCTCAGCTGGCTGCCTCCCAGCCTGGGCCAAAGGT
GCTGTCGGCGGAACAGGGGAGCTACTTCGTTCGTTTAGGTGACCTGGGTCCCAGCTTCCGCCAGCGGG
CATTTGAACACGCGGTGAGCCACCTGCAGCACGGCCAGTTCCAAGCCAGGGACACTCTGGTCCAGCTC
CAGGACTGCTTCAGGCTGATTGAAAAGGCCCAGCAGGCTCCAGAAGGGCAGCCACGTCTGGACCAGGG
CTCAGGTGCCAGTGCGGAGGACGCTGCTGTCCAGGAGAGGGTCTGCGGCCTTCTCCGGCAGCTGCACA
CGGCCTACAGTGGCCTGGTCTCCAGCCTCCAGGGCCTGCCCGCCGAGCTCCAGCAGCCAGTGGGGCGG
GCGCGGCACAGCCTCTGTGAGCTCTATGGCATCGTGGCCTCAGCTGGCTCTGTAGAGGAGCTGCCCGC
AGAGCGGCTGGTGCAGAGCCGCGAGGGTGTGCACCAGGCTTGGCAGGGGTTAGAGCAGCTGCTGGAGG
GCCTACAGCACAATCCCCCGCTCAGCTGGCTGGTAGGGCCCTTCGCCTTGCCCGCTGGCGGGCAGTAG
CTGTAGGAGCCTGCAGGCCCGGCGCGGGGTC

| | | | |
|---|---|---|---|
| NOV32h, | | | |
| SNP13382505 of | | | |
| CG55906-01, | SEQ ID NO: 808 | | MW at 120859.1kD |
| Protein Sequence | SNP Pos: 1088 | 1225 aa | SNP Change: Ala to Val |

MSAPDEGRRDPPKPKGKPPAPMQTLGSFFGSLPGFSSARNLVANAHSSVGAKDLVCSKMSRAKDAVSS
GVASVVDVAKGVVQGGLDTTRSALTGTKEVVSSGVTGAMDMAKGAVQGGLDTSKAVLTGTKDTVSTGL
TGAVNVAKGTVQAGVDTTKTVLTGTKDTVTTGVMGAVNLAKGTVQTGVETSKAVLTGTKDAVSTGLTG
AVNVARGSIQTGVDTSKTVLTGTKDTVCSGVTGAMNVAKGTIQTGVDTSKTVLTGTKDTVCSGVTGAM
NVAKGTIQTGVDTSKTVLTGTKDTVCSGVTGANNVAKGTIQTGVDTTKTVLTGTKNTVCSGVTGAVNL
AKEAIQGGLDTTKSMVMGTKDTMSTGLTGAANVAKGAMQTGLNTTQNIATGTKDTVCSGVTGAMNLAR
GTIQTGVDTTKIVLTGTKDTVCSGVTGAANVAKGAVQGGLDTTKSVLTGTKDAVSTGLTGAVNVAKGT
VQTGVDTTKTVLTGTKDTVCSGVTSAVNVAKGAVQGGLDTTKSVVIGTKDTMSTGLTGAANVAKGAVQ
TGVDTAIKTVLTGTKDTVTTGLVGAVNVAKGTVQTGDTTKTVLTGTKDTIYSGVTSAVNVAKGAVQTG
LKTTQNIATGTKNTFGSGVTSAVNVAXGAAQTGVDTAKTVLTGTKDTVTTGLMGAVNVAKGTVQTSVD
TTKTVLTGTKDTVCSGVTGAANVAKGAQGGLDTTKSVLTGTKDAVSTGLTGAVKLAIKGTVQTGMDTT
KTVLTGTKDAVCSGVTGAANVAKGAVQMGVDTAKTVLTGTKDTVCSGVTGAANVAKGAVQTGLKTTQN
IATGTKNTLGSGVTGAAKVAKGAVQGGLDTTKSVLTGTKDAVSTGLTGAVNLAKGTVQTGVDTSKTVL
TGTKDTVCSGVTGAVNVAKGTVQTGVDTAKTVLSGAXDAVTTGVTGAVNVAKGTVQTGVDASKAVLMG
TKDTVFSGVTGAMSMAKGAVQGGLDTTKTVLTGTKDAVSAGLMGSGNVATGATHTGLSTFQNWLPSTP
ATSWGGLTSSRTTAQLAASQPGPKVLSAEQGSYFVRLGDLGPSFRQRAFEHAVSHLQHGQFQARDTLV
QLQDCFRLIEKAQQAPEGQPRLDQGSGASAEDAAVQERVCGLLRQLHTAYSGLVSSLQGLPAELQQPV
GRARHSLCELYGIVASAGSVEELPAERLVQSREGVHQAWQGLEQLLEGLQHNPPLSWLVGPFALPAGG

| | | |
|---|---|---|
| SNP1 3382504 of | SEQ ID NO: 809 | 3839 bp |
| G55906-01, | ORF Start: ATG at 131 | ORF Stop: TAG at 3806 |
| DNA Sequence | SNP Pos: 3409 | SNP Change: C to T |

GTGAGGCCAGGCCTGCAGGTGGGTGTCGGGCTGCTCAGGCTTTCAGTGGGGAGTGGGTGTGGGATCGG
AGGCTAGGGAACCCCCATTCACGCACCTTCTCTGCCCCCTTCCAGCTTCTCACGTTCTCACTATGTCT
GCTCCAGACGAAGGGAGACGGGATCCCCCCAAACCGAAGGGCAAGCCCCCCGCCCCCATGCAGACCCT
GGGCAGCTTCTTTGGGTCCCTGCCTGGCTTCAGCTCTGCCCGGAACCTGGTGGCCAACGCACATAGCT
CGGTCGGGGCCAAAGACCTGGTGTGTTCCAAGATGTCCAGGGCCAAGGATGCCGTGTCCTCCGGGGTG
GCCAGCGTGGTGGACGTGGCTAAGGGAGTGGTCCAGGGAGGCCTGGACACCACTCGGTCTGCACTTAC
GGGCACCAAGGAGGTGGTGTCCAGCGGGGTCACAGGGGCCATGGACATGGCTAAGGGGGCCGTCCAAG
GGGGTCTGGACACCTCGAAGGCTGTCCTCACCGGCACCAAGGACACGGTGTCCACTGGGCTCACGGGG
GCAGTGAATGTGGCCAAAGGGACCGTACAGGCCGGTGTGGACACCACCAAGACTGTGCTGACCGGCAC
CAAAGACACAGTGACTACTGGGGTCATGGGGGCAGTGAACTTGGCCAAAGGGACTGTCCAGACTGGCG
TGGAAACCTCCAAGGCTGTGCTGACCGGCACCAAAGATGCTGTGTCCACTGGGCTCACAGGGGCAGTG
AATGTGGCCAGAGGAAGCATTCAGACCGGTGTGGACACCAGTAAGACTGTCCTAACAGGTACCAAGGA
CACCGTCTGTAGTGGGGTGACTGGTGCCATGAATGTGGCCAAAGGAACCATCCAGACCGGCGTGGACA
CCAGTAAGACTGTCCTAACAGGTACCAAGGACACCGTCTGTAGTGGGGTGACTGGTGCCATGAATGTG
GCCAAAGGAACCATCCAGACCGGCGTGGACACCAGTAAGACTGTCCTAACAGGTACCAAGGACACCGT
CTGTAGTGGGGTGACTGGTGCCATGAATGTGGCCAAAGGAACCATCCAGACCGGCGTGGACACCACCA
AGACTGTCCTAACTGGCACCAAGAACACTGTCTGCAGTGGGGTGACCGGTGCCGTGAACTTGGCCAAA
GAGGCCATCCAGGGGGGCCTGGATACCACCAAGTCTATGGTCATGGGTACGAAAGACACGATGTCCAC
TGGGCTCACAGGGGCAGCGAATGTGGCCAAGGGGGCCATGCAAACTGGGCTGAACACAACCCAAAATA
TCGCAACAGGTACAAAGGACACCGTCTGCAGTGGGGTGACTGGTGCCATGAATTTGGCCAGAGGAACC
ATCCAGACAGGCGTGGACACCACCAAGATCGTTCTAACTGGTACCAAGGACACTGTCTGCAGTGGGGT
CACCGGTGCTGCGAATGTGGCCAAAGGGGCCGTCCAGGGCGGCCTGGACACTACAAAGTCTGTCCTGA
CTGGCACTAAAGATGCTGTGTCCACTGGGCTCACAGGGGCTGTGAACGTGGCCAAAGGGACCGTCCAG
ACCGGCGTAGACACCACCAAGACTGTCCTAACCGGCACCAAGGACACCGTCTGCAGTGGGGTGACCAG
TGCTGTGAACGTGGCCAAAGGGGCCGTCCAGGGGGGCCTGGACACCACCAAGTCTGTGGTCATAGGTA
CAAAAGACACGATGTCCACTGGGCTCACGGGGGCAGCGAATGTGGCCAAGGGGGCTGTCCAGACAGGT
GTAGACACAGCCAAGACCGTGCTGACCGGCACCAAGGACACAGTGACTACTGGGCTCGTGGGGGCAGT
GAATGTCGCCAAAGGGACCGTCCAGACAGGCATGGACACCACCAAAACTGTCCTAACCGGTACCAAGG
ACACCATCTACAGTGGGGTCACCAGTGCCGTGAACGTGGCCAAGGGGGCTGTGCAAACTGGGCTGAAA
ACGACCCAAAATATCGCGACAGGTACAAAGAACACCTTTGGCAGTGGGGTGACCAGTGCTGTGAATGT
GGCCAAAGGGGCTGCCCAGACAGGTGTAGACACGGCCAAGACCGTGCTGACCGGCACCAAGGACACAG
TCACTACTGGGCTCATGGGGGCAGTGAATGTCGCCAAAGGGACTGTCCAGACCAGTGTGGACACCACC
AAGACTGTCCTAACTGGTACCAAGGACACCGTCTGCAGTGGGGTGACCGGTGCTGCGAATGTGGCCAA
AGGGGCCATCCAAGGGGGCCTGGACACTACAAAGTCTGTCCTGACTGGCACTAAAGATGCTGTGTCCA
CTGGGCTCACAGGGGCTGTGAAGTTGGCCAAAGGGACTGTCCAGACCGGCATGGACACCACCAAGACT
GTGTTAACTGGTACCAAGGATGCTGTGTGCAGTGGGGTGACCGGTGCTGCGAATGTGGCCAAGGGGGC
CGTCCAGATGGGTGTAGACACGGCCAAGACCGTGCTGACCGGTACCAAGGACACTGTCTGCAGTGGGG
TCACCGGTGCTGCGAACGTGGCCAAGGGTGCTGTGCAAACTGGGCTGAAAACGACCCAAAATATCGCA
ACAGGTACAAAGAACACCCTTGGCAGTGGGGTGACCGGTGCTGCGAAAGTGGCCAAAGGGGCCGTCCA
GGGGGGCCTGGACACTACAAAGTCTGTCCTGACTGGCACTAAAGATGCCGTGTCCACTGGGCTCACAG
GGGCTGTGAACTTGGCCAAAGGGACTGTCCAGACCGGCGTGGACACCAGCAAGACTGTCCTGACCAAT
ACCAAGGACACCGTCTGCAGTGGAGTCACTGGTGCCGTAAATGTGGCCAAAGGGACCGTCCAGACAGG
TGTGGACACAGCCAAGACGGTGCTGAGTGGCGCTAAGGATGCAGTGACTACTGGAGTCACGGGGGCAG
TGAATGTGGCCAAAGGAACCGTGCAGACCGGCGTGGACGCCTCCAAGGCTGTGCTTATGGGTACCAAG
GACACTGTCTTCAGTGGGGTTACCGGTGCCATGAGCATGGCCAAAGGGGCCGTCCAGGGGGGCCTGGA
CACCACCAAGACAGTGCTGACCGGAACCAAAGACGCAGTGTCCGCTGGGCTCATGGGGTCAGGGAACG
TGGCGACAGGGGCCACCCACACTGGCCTCAGCACCTTCCAGAACTGGTTACCTAGTACCCCCGCCACC
TCCTGGGGTGGACTCACCAGTTCCAGGACCACAGCTCAGCTGGCTGCCTCCCAGCCTGGGCCAAAGGT
GCTGTCGGCGGAACAGGGGAGCTACTTCGTTCGTTTAGGTGACCTGGGTCCCAGCTTCCGCCAGCGGG
CATTTGAACACGCGGTGAGCCACCTGCAGCACGGCCAGTTCCAAGCCAGGGACACTCTGGCCCAGCTC
CAGGACTGTTTCAGGCTGATTGAAAAGGCCCAGCAGGCTCCAGAAGGGCAGCCACGTCTGGACCAGGG
CTCAGGTGCCAGTGCGGAGGACGCTGCTGTCCAGGAGAGGGTCTGCGGCCTTCTCCGGCAGCTGCACA
CGGCCTACAGTGGCCTGGTCTCCAGCCTCCAGGGCCTGCCCGCCGAGCTCCAGCAGCCAGTGGGGCGG
GCGCGGCACAGCCTCTGTGAGCTCTATGGCATCGTGGCCTCAGCTGGCTCTGTAGAGGAGCTGCCCGC
AGAGCGGCTGGTGCAGAGCCGCGAGGGTGTGCACCAGGCTTGGCAGGGGTTAGAGCAGCTGCTGGAGG
GCCTACAGCACAATCCCCCGCTCAGCTGGCTGGTAGGGCCCTTCGCCTTGCCCGCTGGCGGGCAGTAG
CTGTAGGAGCCTGCAGGCCCGGCGCGGGGTC

| | | | |
|---|---|---|---|
| NOV32i, | | | |
| SNP13382504 of | | | |
| CG559O6-01, | SEQ ID NO: 810 | | MW at 120831.1kD |
| Protein Sequence | SNP Pos: 1093 | 1225 aa | SNP Change: Cys to Cys |

MSAPDEGRRDPPKPKGKPPAPMQTLGSFFGSLPGFSSAENLVANAHSSVGAKDLVCSKMSRAKDAVSS
GVASVVDVAKGVVQGGLDTTRSALTGTKEVVSSGVTGAMDMAKGAVQGGLDTSKAVLTGTKDTVSTGL
TGAVNVAKGTVQAGVDTTKTVLTGTKDTVTTGVMGAVNLAKGTVQTGVETSKAVLTGTKDAVSTGLTG
AVNVARGSIQTGVDTSKTVLTGTKDTVCSGVTGANNVAKGTIQTGVDTSKTVLTGTKDTVCSGVTGAM
NVAKGTIQTGVDTSKTVLTGTKDTVCSGVTGANNVAXGTIQTGVDTTKTVLTGTKNTVCSGVTGAVNL
AKEAIQGGLDTTKSMVMGTKDTMSTGLTGAANVAKGAMQTGLNTTQNIATGTKDTVCSGVTGAMNTAR
GTIQTGVDTTKIVLTGTKDTVCSGVTGAANVAKGAVQGGLDTTKSVLTGTKDAVSTGLTGAVNVAKGT
VQTGVDTTKTVLTGTKDTVCSGVTSAVNVAKGAVQGGLDTTKSVVIGTKDTMSTGLTGAANVAKGAVQ
TGVDTAKTVLTGTKDTVTTGLVGAVNVAKGTVQTGMDTTKTVLTGTKDTIYSGVTSAVNVAKGAVQTG
LKTTQNIATGTKNTFGSGVTSAVVAKGAAQTGVDTAKTXTLTGTKDTVTTGLMGAVNVAKGTVQTSVD
TTKTVLTGTKDTVCSGVTGAANVAKGAIQGGLDTTKSVLTGTKDAVSTGLTGAVKLAKGTVQTGMDTT
KTVLTGTKDAVCSGVTGAAAVAKGAVQMGVDTAKTVLTGTKDTVCSGVTGAAINAKGAVQTGLKTTQN
IATGTKNTLGSGVTGAAKVAKGAVQGGLDTTKSVLTGTKDAVSTGLTGAVNLAKGTVQTGVDTSKTVL
TGTKDTVCSGVTGAVNVAKGTVQTGVDTAKTVLSGAKDAVTTGVTGAVNVAKGTVQTGVDASKAVLMG
TKDTVFSGVTGAMSMAKGAVQGGLDTTKTVLTGTKDAVSAGLMGSGNVATGATHTGLSTFQNWLPSTP
ATSWGGLTSSRTTAQLAASQPGPKVLSAEQGSYFVRLGDLGPSFRQRAFEHAVSHLQHGQFQARDTLA
QLQDCFRLIEKAQQAPEGQPRLDQGSGASAEDAAVQERVCGLLRQLHTAYSGLVSSLQGLPAELQQPV
GRARHSLCELYGIVASAGSVEELPAERLVQSREGVHQAWQGLEQLLEGLQHNPPLSWLVGPFALPAGG
Q

| | | |
|---|---|---|
| NOV32j, | | |
| SNP13376440 of | SEQ ID NO: 811 | 3839 bp |
| CG55906-01, | ORF Start: ATG at 131 | ORF Stop: TAG at 3806 |
| DNA Sequence SNP Pos: 3681 | SNP Change: T to C | |

GTGAGGCCAGGCCTGCAGGTGGGTGTCGGGCTGCTCAGGCTTTCAGTGGGGAGTGGGTGTGGGATGGG
AGGCTAGGGAACCCCCATTCACGCACCTTCTCTGCCCCCTTCCAGCTTCTCACGTTCTCACTATGTCT
GCTCCAGACGAAGGGAGACGGGATCCCCCCAAACCGAAGGGCAAGCCCCCCGCCCCCATGCAGACCCT
GGGCAGCTTCTTTGGGTCCCTGCCTGGCTTCAGCTCTGCCCGGAACCTGGTGGCCAACGCACATAGCT
CGGTCGGGGCCAAAGACCTGGTGTGTTCCAAGATGTCCAGGGCCAAGGATGCCGTGTCCTCCGGGGTG
GCCAGCGTGGTGGACGTGGCTAAGGGAGTGGTCCAGGGAGGCCTGGACACCACTCGGTCTGCACTTAC
GGGCACCAAGGAGGTGGTGTCCAGCGGGGTCACAGGGGCCATGGACATGGCTAAGGGGGCCGTCCAAG
GGGGTCTGGACACCTCGAAGGCTGTCCTCACCGGCACCAAGGACACGGTGTCCACTGGGCTCACGGGG
GCAGTGAATGTGGCCAAAGGGACCGTACAGGCCGGTGTGGACACCACCAAGACTGTGCTGACCGGCAC
CAAAGACACAGTGACTACTGGGGTCATGGGGGCAGTGAACTTGGCCAAAGGGACTGTCCAGACTGGCG
TGGAAACCTCCAAGGCTGTGCTGACCGGCACCAAAGATGCTGTGTCCACTGGGCTCACAGGGGCAGTG
AATGTGGCCAGAGGAAGCATTCAGACCGGTGTGGACACCAGTAAGACTGTCCTAACAGGTACCAAGGA
CACCGTCTGTAGTGGGGTGACTGGTGCCATGAATGTGGCCAAAGGAACCATCCAGACCGGCGTGGACA
CCAGTAAGACTGTCCTAACAGGTACCAAGGACACCGTCTGTAGTGGGGTGACTGGTGCCATGAATGTG
GCCAAAGGAACCATCCAGACCGGCGTGGACACCAGTAAGACTGTCCTAACAGGTACCAAGGACACCGT
CTGTAGTGGGGTGACTGGTGCCATGAATGTGGCCAAAGGAACCATCCAGACCGGCGTGGACACCACCA
AGACTGTCCTAACTGGCACCAAGAACACTGTCTGCAGTGGGGTGACCGGTGCCGTGAACTTGGCCAAA
GAGGCCATCCAGGGGGGCCTGGATACCACCAAGTCTATGGTCATGGGTACGAAAGACACGATGTCCAC
TGGGCTCACAGGGGCAGCGAATGTGGCCAAGGGGGCCATGCAAACTGGGCTGAACACAACCCAAAATA
TCGCAACAGGTACAAAGGACACCGTCTGCAGTGGGGTGACTGGTGCCATGAATTTGGCCAGAGGAACC
ATCCAGACAGGCGTGGACACCACCAAGATCGTTCTAACTGGTACCAAGGACACTGTCTGCAGTGGGGT
CACCGGTGCTGCGAATGTGGCCAAAGGGGCCGTCCAGGGCGGCCTGGACACTACAAAGTCTGTCCTGA
CTGGCACTAAAGATGCTGTGTCCACTGGGCTCACAGGGGCTGTGAACGTGGCCAAAGGGACCGTCCAG
ACCGGCGTAGACACCACCAAGACTGTCCTAACCGGCACCAAGGACACCGTCTGCAGTGGGGTGACCAG
TGCTGTGAACGTGGCCAAAGGGGCCGTCCAGGGGGGCCTGGACACCACCAAGTCTGTGGTCATAGGTA
CAAAAGACACGATGTCCACTGGGCTCACGGGGGCAGCGAATGTGGCCAAGGGGGCTGTCCAGACAGGT
GTAGACACAGCCAAGACCGTGCTGACCGGCACCAAGGACACAGTGACTACTGGGCTCGTGGGGGCAGT
GAATGTCGCCAAAGGGACCGTCCAGACAGGCATGGACACCACCAAAACTGTCCTAACCGGTACCAAGG
ACACCATCTACAGTGGGGTCACCAGTGCCGTGAACGTGGCCAAGGGGGCTGTGCAAACTGGGCTGAAA
ACGACCCAAAATATCGCGACAGGTACAAAGAACACCTTTGGCAGTGGGGTGACCAGTGCTGTGAATGT
GGCCAAAGGGGCTGCCCAGACAGGTGTAGACACGGCCAAGACCGTGCTGACCGGCACCAAGGACACAG
TCACTACTGGGCTCATGGGGGCAGTGAATGTCGCCAAAGGGACTGTCCAGACCAGTGTGGACACCACC
ATGACTGTCCTAACTGGTACCAAGGACACCGTCTGCAGTGGGGTGACCGGTGCTGCGAATGTGGCCAA
AGGGGCCATCCAAGGGGGCCTGGACACTACAAAGTCTGTCCTGACTGGCACTAAAGATGCTGTGTCCA
CTGGGCTCACAGGGGCTGTGAAGTTGGCCAAAGGGACTGTCCAGACCGGCATGGACACCACCAAGACT
GTGTTAACTGGTACCAAGGATGCTGTGTGCAGTGGGGTGACCGGTGCTGCGAATGTGGCCAAGGGGGC
CGTCCAGATGGGTGTAGACACGGCCAAGACCGTGCTGACCGGTACCAAGGACACTGTCTGCAGTGGGG
TCACCGGTGCTGCGAACGTGGCCAAGGGTGCTGTGCAAACTGGGCTGAAAACGACCCAAAATATCGCA
ACAGGTACAAAGAACACCCTTGGCAGTGGGGTGACCGGTGCTGCGAAAGTGGCCAAAGGGGCCGTCCA
GGGGGGCCTGGACACTACAAAGTCTGTCCTGACTGGCACTAAAGATGCCGTGTCCACTGGGCTCACAG
GGGCTGTGAACTTGGCCAAAGGGACTGTCCAGACCGGCGTGGACACCAGCAAGACTGTCCTGACCGGT
ACCAAGGACACCGTCTGCAGTGGAGTCACTGGTGCCGTAAATGTGGCCAAAGGGACCGTCCAGACAGG
TGTGGACACAGCCAAGACGGTGCTGAGTGGCGCTAAGGATGCAGTGACTACTGGAGTCACGGGGGCAG
TGAATGTGGCCAAAGGAACCGTGCAGACCGGCGTGGACGCCTCCAAGGCTGTGCTTATGGGTACCAAG
GACACTGTCTTCAGTGGGGTTACCGGTGCCATGAGCATGGCCAAAGGGGCCGTCCAGGGGGGCCTGGA
CACCACCAAGACAGTGCTGACCGGAACCAAAGACGCAGTGTCCGCTGGGCTCATGGGGTCAGGGAACG
TGGCGACAGGGGCCACCCACACTGGCCTCAGCACCTTCCAGAACTGGTTACCTAGTACCCCCGCCACC
TCCTGGGGTGGACTCACCAGTTCCAGGACCACAGCTCAGCTGGCTGCCTCCCAGCCTGGGCCAAAGGT
GCTGTCGGCGGAACAGGGGAGCTACTTCGTTCGTTTAGGTGACCTGGGTCCCAGCTTCCGCCAGCGGG
CATTTGAACACGCGGTGAGCCACCTGCAGCACGGCCAGTTCCAAGCCAGGGACACTCTGGCCCAGCTC
CAGGACTGTTTCAGGCTGATTGAAAAGGCCCAGCAGGCTCCAGAAGGGCAGCCACGTCTGGACCAGGG
CTCAGGTGCCAGTGCGGAGGACGCTGCTGTCCAGGAGAGGGTCTGCGGCCTTCTCCGGCAGCTGCACA
CGGCCTACAGTGGCCTGGTCTCCAGCCTCCAGGGCCTGCCCGCCGAGCTCCAGCAGCCAGTGGGGCGG
GCGCGGCACAGCCTCTGTGAGCTCTATGGCATCGTGGCCTCAGCTGGCTCTGTAGAGGAGCTGCCCGC
AGAGCGGCCGGTGCAGAGCCGCGAGGGTGTGCACCAGGCTTGGCAGGGGTTAGAGCAGCTGCTGGAGG
GCCTACAGCACAATCCCCCGCTCAGCTGGCTGGTAGGGCCCTTCGCCTTGCCCGCTGGCGGGCAGTAG
CTGTAGGAGCCTGCAGGCCCGGCGCGGGGTC

| | | | |
|---|---|---|---|
| NOV32j, | | | |
| SNP13376440 of | | | |
| CG55906-01, | SEQ ID NO: 812 | | MW at 120815.0kD |
| Protein Sequence | SNP Pos: 1184 | 1225 aa | SNP Change: Leu to Pro |

MSAPDEGRPDPPKPKGKPPAPMQTLGSFFGSLPGFSSARNLVANAHSSVGAKDLVCSKMSRAKDAVSS
GVASVVDVAXGVVQGGLDTTRSALTGTKEVVSSGVTGANDMAKGAVQGGLDTSKAVLTGTKDTVSTGL
TGAVNVAKGTVQAGVDTTKTVLTGTKDTVTTGVMGAVNLAKGTVQTGVETSKAVLTGTKDAVSTGLTG
AVNVARGSIQTGVDTSKTVLTGTKDTVCSGVTGAMNVAKGTIQTGVDTSKTVLTGTKDTVCSGVTGAM
NVAKGTIQTGVDTSKTVLTGTKDTTCSGVTGAMNVAKGTIQTGVDTTKTVLTGTKNTVCSGVTGAVNL
AKEAIQGGLDTTKSMVMGTKDTMSTGLTGAANVAKGAMQTGLNTTQNIATGTKDTVCSGVTGANNLAR
GTIQTGVDTTKIVLTGTKDTVCSGVTGAANVAKGAVQGGLDTTKSVLTGTKDAVSTGLTGAVNVAKGT
VQTGVDTTKTVLTGTKDTVCSGVTSAVNVAKGAVQGGLDTTKSVVIGTKDTMSTGLTGAANVAXGAVQ
TGVDTAKTVLTGTKDTVTTGLVGAVNVAKGTVQTGMDTTKTVLTGTKDTIYSGVTSAVNVAKGAVQTG
LKTTQNIATGTKNTFGSGVTSAVNVAKGAAQTGVDTAKTVLTGTKDTVTTGLMGAVNVAKGTVQTSVD
TTKTVLTGTKDTVCSGVTGAANVAKGATQGGLDTTKSVLTGTKDAVSTGLTGAVKLAKGTVQTGMDTT
KTVLTGTKDAVCSGVTGAANVAKGAVQMGVDTAKTVLTGTKDTVCSGVTGAANVAKGAVQTGLKTTQN
IATGTKNTLGSGVTGAAKVAKGAVQGGLDTTKSVLTGTKDAVSTGLTGAVNLAKGTVQTGVDTSKTVL
TGTKDTVCSGVTGAVNVAKGTVQTGVDTAKTVLSGAKDAVTTGVTGAVNVAKGTVQTGVDASKAVLMG
TKDTVFSGVTGAMSMAKGAVQGGLDTTKTVLTGTKDAVSAGLMGSGNVATGATHTGLSTFQNWLPSTP
ATSWGGLTSSRTTAQLAASQPGPKTLSAEQGSYFVRLGDLGPSFRQPAFEHAVSHLQHGQFQARDTLA
QLQDCFRLIEKAQQAPEGQPRLDQGSGASAEDAAVQERVCGLLRQLHTAYSGLVSSLQGLPAELQQPV
GRARHSLCELYGIVASAGSVEELPAERPVQSREGVHQAWQGLEQLLEGLQHNPPLSWLVGPFALPAGG
Q

A ClustalW comparison of the above protein sequences yields the following sequence alignment shown in Table 32B.

TABLE 32B
Comparison of the NOV32 protein sequences.

| | | |
|---|---|---|
| NOV32a | ------------------------------------------------------------ | (SEQ ID NO:794) |
| NOV32b | MSAPDEGRRDPPKPKGKTLGSFFGSLPGFNSARNLVANAHSSARARPAADPTGAPAAEAA | (SEQ ID NO:796) |
| NOV32c | ------------------------------------------------------------ | (SEQ ID NO:798) |
| NOV32d | ------------------------------------------------------------ | (SEQ ID NO:800) |
| NOV32e | ------------------------------------------------------------ | (SEQ ID NO:802) |
| NOV32f | ------------------------------------------------------------ | (SEQ ID NO:804) |
| NOV32g | ------------------------------------------------------------ | (SEQ ID NO:806) |
| NOV32a | ------------------------------------------------------------ | |
| NOV32b | QPQAQVAAHPEQTAPWTEKELQPSEKQMVSGAKDLVCSKNSRAKDAVSSGVASVVDVAKG | |
| NOV32c | ------------------------------------------------------------ | |
| NOV32d | ------------------------------------------------------------ | |
| NOV32e | ------------------------------------------------------------ | |
| NOV32f | ------------------------------------------------------------ | |
| NOV32g | ------------------------------------------------------------ | |
| NOV32a | ------------------------------------------------------------ | |
| NOV32b | VVQGGLDTTRSALTGTKEAVSSGVTGAMDMAKGAVQGGLDTSKAVLTGTKDTVSTGLTGA | |
| NOV32c | ------------------------------------------------------------ | |
| NOV32d | ------------------------------------------------------------ | |
| NOV32e | ------------------------------------------------------------ | |
| NOV32f | ------------------------------------------------------------ | |
| NOV32g | ------------------------------------------------------------ | |
| NOV32a | ------------------------------------------------------------ | |
| NOV32b | VNVAKGTVQAGVDTTKTVLTGTKDTVTTGVMGAVNLAKGTVQTGVETSKAVLTGTKDAVS | |
| NOV32c | ------------------------------------------------------------ | |
| NOV32d | ------------------------------------------------------------ | |
| NOV32e | ------------------------------------------------------------ | |
| NOV32f | ------------------------------------------------------------ | |
| NOV32g | ------------------------------------------------------------ | |
| NOV32a | ------------------------------------------------------------ | |
| NOV32b | TGLTGAVNVARGSIQTGVDTSKTVLTGTKDTVCSGVTSAMNVAKGTIQTGVDTSKTVLTG | |
| NOV32c | ------------------------------------------------------------ | |
| NOV32d | ------------------------------------------------------------ | |
| NOV32e | ------------------------------------------------------------ | |
| NOV32f | ------------------------------------------------------------ | |
| NOV32g | ------------------------------------------------------------ | |
| NOV32a | ------------------------------------------------------------ | |
| NOV32b | TKDTVCSGVTGAMNVAKGTIQTGVDTSKTVLTGTKDTVCSGVTGAMNVAKGTIQTGVDTT | |
| NOV32c | ------------------------------------------------------------ | |
| NOV32d | ------------------------------------------------------------ | |
| NOV32e | ------------------------------------------------------------ | |
| NOV32f | ------------------------------------------------------------ | |
| NOV32g | ------------------------------------------------------------ | |
| NOV32a | ------------------------------------------------------------ | |
| NOV32b | KTVLTGTKNTVCSGVTGAVNLAKEAIQGGLDTTKSMVMGTKDTMSTGLTGAANVAKGAMQ | |
| NOV32c | ------------------------------------------------------------ | |
| NOV32d | ------------------------------------------------------------ | |
| NOV32e | ------------------------------------------------------------ | |
| NOV32f | ------------------------------------------------------------ | |
| NOV32g | ------------------------------------------------------------ | |
| NOV32a | ------------------------------------------------------------ | |
| NOV32b | TGLNTTQNIATGTKDTVCSGVTGANNLARGTIQTGVDTTKIVLTGTKDTVCSGVTGAANV | |
| NOV32c | ------------------------------------------------------------ | |
| NOV32d | ------------------------------------------------------------ | |
| NOV32e | ------------------------------------------------------------ | |
| NOV32f | ------------------------------------------------------------ | |
| NOV32g | ------------------------------------------------------------ | |
| NOV32a | ------------------------------------------------------------ | |
| NOV32b | AKGAVQGGLDTTKSVLTGTKDAVSTGLTGAVNVAKGTVQTGVDTTKTVLTGTKDTVCSGV | |
| NOV32c | ------------------------------------------------------------ | |
| NOV32d | ------------------------------------------------------------ | |
| NOV32e | ------------------------------------------------------------ | |
| NOV32f | ------------------------------------------------------------ | |
| NOV32g | ------------------------------------------------------------ | |
| NOV32a | ------------------------------------------------------------ | |
| NOV32b | TSAVNVAKGAVQGGLDTTKSVVIGTKDTMSTGLTGAANVAKGAVQTGVDTAKTVLTGTKD | |
| NOV32c | ------------------------------------------------------------ | |
| NOV32d | ------------------------------------------------------------ | |
| NOV32e | ------------------------------------------------------------ | |
| NOV32f | ------------------------------------------------------------ | |
| NOV32g | ------------------------------------------------------------ | |
| NOV32a | ------------------------------------------------------------ | |
| NOV32b | TVTTGLVGAVNVAKGTVQTGMDTTKTVLTGTKDTIYSGVTSAVNVAKGAVQTGLKTTQNI | |
| NOV32c | ------------------------------------------------------------ | |
| NOV32d | ------------------------------------------------------------ | |
| NOV32e | ------------------------------------------------------------ | |
| NOV32f | ------------------------------------------------------------ | |
| NOV32g | ------------------------------------------------------------ | |
| NOV32a | ----------------------------------MSAPDEGRRDPPKPKGK------TLG | |
| NOV32b | ATGTKNTFGSGVTGAVNVAKGAVQTGVDTAKTVLTGTKDTVTTGLMGAVNV------AKG | |
| NOV32c | ----------------------------------MSAPDEGRPDPPKPKGKPPAPMQTLG | |
| NOV32d | -------------------------------RSIMSAPDEGRRDPPKPKGK------TLG | |
| NOV32e | ----------------------------------MSAPDEGRRDPPKPKGK------TLG | |
| NOV32f | ---------------------------ARPPFTTMSAPDEGRPDPPKPKGK------TLG | |
| NOV32g | ----------------------------------MSAPDEGRRDPPKPKGK------TLG | |
| NOV32a | SFFGSLPGFSSARNLVANAHSS-----------ARARPAADPTGAPAAEAAQPQAQVAAH | |
| NOV32b | TVQTSVDTTKTVLTGTKDTVCS-----------GVTGAANVAKGAVQTGVDTAKTVLTGT | |
| NOV32c | SFFGSLPGFSSAPNLVANAHSSVGAKDLVCSKMSRAKDAVSSGVASVVDVAKGVVQGGLD | |
| NOV32d | SFFGSLPGFSSARNLVANAHSS-----------ARARPAADPTGAPAAEAAQPQAQVAAH | |
| NOV32e | SFFGSLPGFNSARNLVANAHSS-----------ARARPAADPTGAPAAEAAQPQAQVAAH | |
| NOV32f | SFFGSLPGFSSARNLVANAHSS-----------ARARPAADPTGAPAAEAAQPQAQVAAH | |
| NOV32g | SFFGSLPGFSSAPNLVANAHSS-----------ARARPAADPTGAPAAEAAQPQAQVAAH | |
| NOV32a | PEQTAPWTEKELQPSEKQ--------MVSGAKDLVCSKMSRAKDAVSSGVASVVDVAKGV | |
| NOV32b | KDTVCSGVTGAVNVAKG---------AVQTGLKTTQNIATGTKNTLGSGVTGAANVAKGA | |
| NOV32c | TTRSALTGTKEVVSSGVTGAMDMAKGAVQGGLDTSKAVLTGTKDTVSTGLTGAVNVAKGT | |
| NOV32d | PEQTAPWTEKELQPSEK---------MVSGAKDLVCSKMSRAYDAVSSGVASVVDVATGV | |
| NOV32e | PEQTAPWTEKELQPSE-------------------------------------------- | |
| NOV32f | PEQTAPWTEKELQPSE-------------------------------------------- | |
| NOV32g | PEQTAPWTEKELQPSE-------------------------------------------- | |
| NOV32a | VQGGLDTTRSALTGTKEVVSSGVTGAMDMAKGAVQGGLDTSKAVLTGTKDTVSTGLTGAV | |
| NOV32b | VQGGLDTTKSVLTGTKDAVSTGLTGAVNLAKGTVQTGMDTTKTVLTGTKDAVCSGVTGAA | |
| NOV32c | VQAGVDTTKTVLTGTKDTVTTGVMGAVNLAXGTVQTGVETSKAVLTGTKDAVSTGLTGAV | |
| NOV32d | VQGGLDTTRSALTGTKEAVSSGFTGAMDMAKGAVQGGLDTSKAVLTGTKDTVSTGLTGAV | |
| NOV32e | ------------------------------------------------------------ | |
| NOV32f | ------------------------------------------------------------ | |
| NOV32g | ------------------------------------------------------------ | |
| NOV32a | NVAKGTVQAGVDTTKTVLTGTKDTVTTGVMGAVNLAKGTVQTGVETSKAVLTGTKDAVST | |
| NOV32b | NVAKGAVQTGVDTAKTVLTGTKDTVTTGLMGAVNVAKGTVQTSVDTTKTVLTGTKDTVCS | |
| NOV32c | NVARGSIQTGVDTSKTVLTGTKDTVCSGVTGAMNVAKGTIQTGVDTSKTVLTGTKDTVCS | |
| NOV32d | NVAKGTVQAGVDTTKTVLTGTKDTVTTGVMGAVNLAKGTVQTGVETSKAVLTGTKDAVST | |
| NOV32e | ------------------------------------------------------------ | |
| NOV32f | ------------------------------------------------------------ | |
| NOV32g | ------------------------------------------------------------ | |
| NOV32a | GLTGAVNVARGSIQTGVDTSKTVLTGTKDTVCSGVTGAMNVAKGTIQTGVDTSKTVLTGT | |
| NOV32b | GVTGAANWAKGAVQGGLDTTKSVLTGTKDTVSTGLTGAVNLAKGTVQTGVDTSKTVLTGT | |
| NOV32c | GVTGAMNVAKGTIQTGVDTSKTVLTGTKDTVCSGVTGAMNVAKGTIQTGVDTTKTVLTGT | |
| NOV32d | GLTGAVNVARGSIQTGVDTSKTVLTGTKDTVCSGVTSAMNNAKGTIQTGVDTSKTVLTGT | |
| NOV32e | ------------------------------------------------------------ | |
| NOV32f | ------------------------------------------------------------ | |
| NOV32g | ------------------------------------------------------------ | |
| NOV32a | KDTVCSGVTGAMNVAKGTIQTGVDTSKTVLTGTKDTVCSGVTGAMNVAKGTIQTGVDTTK | |
| NOV32b | KDTVCSGVT--------------------------------------------------- | |
| NOV32c | KNTVCSGVTGAVNLAKEAIQGGLDTTKSMVMGTKDTMSTGLTGAANVAKGAMQTGLNTTQ | |
| NOV32d | KDTVCSGVT--------------------------------------------------- | |
| NOV32e | ------------------------------------------------------------ | |
| NOV32f | ------------------------------------------------------------ | |
| NOV32g | ------------------------------------------------------------ | |
| NOV32a | TVLTGTKNTVCSGVTGAVNLAKEAIQGGLDTTKSMVMGTKDTMSTGLTGAANVAKGAMQT | |
| NOV32b | ------------------------------------------------------------ | |
| NOV32c | NIATGTKDTVCSGVTGAMNLARGTIQTGVDTTKIVLTGTKDTVCSGVTGAANVAKGAVQG | |
| NOV32d | ------------------------------------------------------------ | |
| NOV32e | ------------------------------------------------------------ | |
| NOV32f | ------------------------------------------------------------ | |
| NOV32g | ------------------------------------------------------------ | |
| NOV32a | GLNTTQNIATGTKDTVCSGVTGAMNLARGTIQTGVDTTKIVLTGTKDTVCSGVTGAANVA | |
| NOV32b | ------------------------------------------------------------ | |
| NOV32c | G---LDTTKSVLTG---------------------------------------------- | |
| NOV32d | ------------------------------------------------------------ | |
| NOV32e | ------------------------------------------------------------ | |
| NOV32f | ------------------------------------------------------------ | |
| NOV32g | ------------------------------------------------------------ | |
| NOV32a | KGAVQGGLDTTKSVLTGTKDAVSTGLTGAVNVAKGTVQTGVDTTKTVLTGTKDTVCSGVT | |
| NOV32b | ------------------------------------------------------------ | |
| NOV32c | ------------------------------------------------------------ | |
| NOV32d | ------------------------------------------------------------ | |
| NOV32e | ------------------------------------------------------------ | |
| NOV32f | ------------------------------------------------------------ | |
| NOV32g | ------------------------------------------------------------ | |
| NOV32a | SAVNVAKGAVQGGLDTTKSVVIGTKDTMSTGLTGAANVAKGAVQTGVDTAKTVLTGTKDT | |
| NOV32b | ------------------------------------------------------------ | |
| NOV32c | --------------------------------------------------------TKDA | |
| NOV32d | ------------------------------------------------------------ | |
| NOV32e | ------------------------------------------------------------ | |
| NOV32f | ------------------------------------------------------------ | |
| NOV32g | ------------------------------------------------------------ | |
| NOV32a | VTTGLVGAVNVAKGTVQTGMDTTKTVLTGTKDTIYSGVTSAVNVAKGAVQTGLKTTQNIA | |
| NOV32b | ------------------------------------------------------------ | |
| NOV32c | VSTGLTGAVNVAKGTVQTGVDTTKTVLTGTKDTVCSGVTSAVNVAKGAVQGGLDTTKSVV | |
| NOV32d | ------------------------------------------------------------ | |
| NOV32f | ------------------------------------------------------------ | |
| NOV32g | ------------------------------------------------------------ | |
| NOV32a | TGTKNTFGSGVTSAVNVAKGAAQTGVDTAKTVLTGTKDTVTTGLMGAVNVAKGTVQTSVD | |
| NOV32b | ------------------------------------------------------------ | |
| NOV32c | IGTKDTMSTGLTGAANVAKGAVQTGVDTAKTVLTGTKDTVTTGLVGAVNVAKGTVQTGMD | |
| NOV32d | ------------------------------------------------------------ | |
| NOV32e | ------------------------------------------------------------ | |
| NOV32f | ------------------------------------------------------------ | |
| NOV32g | ------------------------------------------------------------ | |
| NOV32a | TTKTVLTGTKDTVCSGVTGAANVAKGAIQGGLDTTKSVLTGTKDAVSTGLTGAVKLAKGT | |
| NOV32b | ------------------------------------------------------------ | |
| NOV32c | TTKTVLTGTKDTIYSGVTSAVNVAKGAVQTGLKTTQNIATGTKNTFGSGVTSAVNVAKGA | |
| NOV32d | ------------------------------------------------------------ | |
| NOV32e | ------------------------------------------------------------ | |
| NOV32f | ------------------------------------------------------------ | |
| NOV32g | ------------------------------------------------------------ | |
| NOV32a | VQTGMDTTKTVLTGTKDAVCSGVTGAANVAKGAVQMGVDTAKTVLTGTKDTVCSGVTGAA | |
| NOV32b | ------------------------------------------------------------ | |
| NOV32c | AQTGVDTAKTVLTGTKDTVTTGLMGAVNVAKGTVQTSVDTTKTVLTGTKDTVCSGVTGAA | |
| NOV32d | ------------------------------------------------------------ | |
| NOV32e | ------------------------------------------------------------ | |
| NOV32f | ------------------------------------------------------------ | |
| NOV32g | ------------------------------------------------------------ | |
| NOV32a | NVAKGAVQTGLKTTQNTATGTKNTLGSGVTGAAKVAKGAVQGGLDTTKSVLTGTKDAVST | |
| NOV32b | ------------------------------------------------------------ | |
| NOV32c | NVAKGAIQGGLDTTKSVLTGTKDAVSTGLTGAVKLAKGTVQTGMDTTKTVLTGTKDAVCS | |
| NOV32d | ------------------------------------------------------------ | |
| NOV32e | ------------------------------------------------------------ | |
| NOV32f | ------------------------------------------------------------ | |
| NOV32g | ------------------------------------------------------------ | |
| NOV32a | GLTGAVNLAKGTVQTGVDTSKTVLTGTKDTVCSGVTGAVNVAKGTVQTGVDTAKTVLSGA | |
| NOV32b | ------------------------------------GAVNVAKGTVQTGVDTAKTVLSGA | |
| NOV32c | GVTGAANVAKGAVQMGVDTAKTVLTGTKDTVCSGVTGAANVAKGAVQTGLKTTQNIATGT | |
| NOV32d | ------------------------------------GAVNVAKGTVQTGVDTAKTVLSGA | |
| NOV32e | ------------------------------------------------------------ | |
| NOV32f | ------------------------------------------------------------ | |
| NOV32g | ------------------------------------------------------------ | |
| NOV32a | KDAVTTGVTGAVNVAKGTVQTGVDASKAVLMGTKDTVFSGVTGAMSMAKGAVQGGLDTTK | |
| NOV32b | KDAVTTGVTGAVNVAKGTVQTGVDASKAVLMGTKDTVFSGVTGAMSMAKGAVQGGLDTTK | |
| NOV32c | KNTLGSGVTGAAKVAKGAVQGGLDTTKSVLTGTKDAVSTGLTGAVNLAKGTVQTGVDTSK | |
| NOV32d | KDAVTTGVTGAVNVAKGTVQTGVDASKAVLMGTKDTVFSGVTGAMSMAKGAVQGGLDTTK | |
| NOV32e | ------------------------------------------------------------ | |
| NOV32f | ------------------------------------------------------------ | |
| NOV32a | TVLTGTKDAVSAGLMGSGNVATGATHTGLSTFQNWLPSTPATSWGGLTSSRTTDNGGEQT | |
| NOV32b | TVLTGTKDAVSAGLMGSGNVATGATHTGLSTFQNWLPSTPATSWGGLTSSRTTDNGGEQT | |
| NOV32c | TVLTGTKDTVCSGVTGAVNVAKGTVQTGVDTAKTVLSGAKDAVTTGVTGAVNVAKGTVQT | |
| NOV32d | TVLTGTKDAVSAGLMGSGNVATGATHTGLSTFQNWLPSTPATSWGGLTSSRTTDNGGEQT | |
| NOV32e | ------------------------------------------------------------ | |
| NOV32f | ------------------------------------------------------------ | |
| NOV32g | ------------------------------------------------------------ | |
| NOV32a | ALSPQEAPFSGISTPPDVLSVGPEPAWEAAATTKGLATDVATFTQGAAPGREDTGL-LAT | |
| NOV32b | ALSPQEAPFSGISTPPDVLSVGPEPAWEAAATTKGLATDVATFTQGAAPGREDTGL-LTT | |
| NOV32c | GVDASKAVLMGTKDTVFSGVTGAMSMAKGAVQGGLDTTKTVLTGTKDAVSAGLMGSGNVA | |
| NOV32d | ALSPQEAPFSGISTPPDVLSVGPEPAWEAAATTKGLATDVATFTQGAAPGREDTGL-LTT | |
| NOV32e | ------------------------------------------------------------ | |
| NOV32f | ------------------------------------------------------------ | |
| NOV32g | ------------------------------------------------------------ | |
| NOV32a | THGPEEAPRLAMLQNELEGLGDTFHPNNAEEQAQLAASQPGPKVLSAEQGSYFVRLGDLG | |
| NOV32b | THGPEEAPRLAMLQNELEGLGDIFHPNNAEEQAQLAASQPGPKVLSAEQGSYFVRLGDLG | |
| NOV32c | TGATHTGLSTFQNWLPSTPATSWGGLTSSRTTAQLAASQPGPKVLSAEQGSYFVRLGDLG | |
| NOV32d | THGPEEAPRLAMLQNELEGLGDIFHPMNAEEQAQLAASQPGPKVLSAEQGSYFVRLGDLG | |
| NOV32e | ------------------------------------------------------------ | |
| NOV32f | ------------------------------------------------------------ | |
| NOV32g | ------------------------------------------------------------ | |
| NOV32a | PSFRQRAFEHAVSHLQHGQFQARDTLAQLQDCFRLIEKAQQAPEGQPRLDQGSGASAEDA | |
| NOV32b | PSFRQRAFEHAVSHLQHGQFQARDTLAQLQDCFRLIEKAQQAPEGQPRLDQGSGASAEDA | |
| NOV32c | PSFRQRAFEHAVSHLQHGQFQARDTLAQLQDCFRLIEKAQQAPEGQPRLDQGSGASAEDA | |
| NOV32d | PSFRQRAFEHAVSHLQHGQFQARDTLAQLQDCFRLIEKAQQAPEGQPRLDQGSGASAEDA | |
| NOV32e | ----------------------------------KIEKAQQAPEGQPRLDQGSGASAEDA | |
| NOV32f | ----------------------------------KIEKAQQAPEGQPRLDQGSGASAEDA | |
| NOV32g | ----------------------------------KIEKAQQAPEGQPRLDQGSGASAEDA | |
| NOV32a | AVQEERDAGVLSRVCGLLRQLHTAYSGLVSSLQGLPAELQQPVGRARHSLCELYGIVASA | |
| NOV32b | AVQEERDAGVLSRVCGLLRQLHTAYSGLVSSLQGLPAELQQPVGRARHSLCELYGIVASA | |
| NOV32c | AVQER--------VCGLLRQLHTAYSGLVSSLQGLPAELQQPVGRARHSLCELYGIVASA | |
| NOV32d | AVQEERDAGVLSRVCGLLRQLHTAYSGLVSSLQGLPAELQQPVGRARHSLCELYGIVASA | |
| NOV32e | AVQEERDAGVLSRVCGLLRQLHTAYSGLVSSLRGLPAELQQPVGRARHSLCELYGIVASA | |
| NOV32f | AVQEERDAGVLSRVCGLLRQLHTAYSGLVSSLQGLPAELQQPVGRARHSLCELYGIVASA | |
| NOV32g | AVQEERDAGVLSRVCGLLRQLHTAYSGLVSSLQGLPAELQQPVGRARHSLCELYGIVASA | |
| NOV32a | GSVEELPAERLVQSREGVHQAWQGLEQLLEGLQHNPPLSWLVGPFALPAGGQ-- | |
| NOV32b | GSVEELPAERLVQSREGVHQAWQGLEQLLEGLQHNPPLSWLVGPFALPAGGQ-- | |
| NOV32c | GSVEELPAERLVQSREGVHQAWQGLEQLLEGLQHNPPLSWLVGPFALPAGGQ-- | |
| NOV32d | GSVEELPAERLVQSREGVHQAWQGLEQLLEGLQHNPPLSWLVGPFALPAGGQLE | |
| NOV32e | GSVEELPAERLVQSREGVHQAWQGLEQLLEGLQHNPPLSWLVGPFALPAGGQ-- | |
| NOV32f | GSVEELPAERLVQSREGVHQAWQGLEQLLEGLQHNPPLSWLVGPFALPAGGQ-- | |
| NOV32g | GSVEELPAERLVQSREGVHQAWQGLEQLLEGLQHNPPLSWLVGPFALPAGGQ-- | |

Further analysis of the NOV32a protein yielded the following properties shown in Table 32C.

TABLE 32C
Protein Sequence Properties NOV32a

| | |
|---|---|
| SignalP analysis: | No Known Signal Sequence Predicted |
| PSORT II analysis: | |

| PSG: | a new signal peptide prediction method | |
|---|---|---|
| | N-region: | length 10; pos. chg 2; neg. chg 3 |
| | H-region: | length 2; peak value 0.00 |
| | PSG score: | −4.40 |

| | |
|---|---|
| GvH: | von Heijne's method for signal seq. recognition |
| | GvH score (threshold: −2.1): −4.76 |
| | possible cleavage site: between 32 and 33 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 0 | |
| | number of TMS(s) . . . fixed | |
| | PERIPHERAL | Likelihood = 1.43 (at 106) |

| | ALOM score: 1.43 (number of TMSs: 0) | | | |
|---|---|---|---|---|
| MITDISC: | discrimination of mitochondrial targeting seq | | | |
| | R content: | 0 | Hyd Moment(75): | 6.59 |
| | Hyd Moment(95): | 5.57 | G content: | 0 |
| | D/E content: | 2 | S/T content: | 1 |

| | |
|---|---|
| | Score: −6.74 |
| Gavel: | prediction of cleavage sites for mitochondrial preseq |
| | cleavage site motif not found |

NUCDISC: discrimination of nuclear localization signals

| | |
|---|---|
| | pat4: none |
| | pat7: none |
| | bipartite: none |
| | content of basic residues: 8.6% |
| | NLS Score: −0.47 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear |
| | discrimination |
| | Prediction: cytoplasmic |
| | Reliability: 94.1 |
| COIL: | Lupas's algorithm to detect coiled-coil regions |
| | total: 0 residues |

Final Results (k = 9/23):

56.5%: cytoplasmic
26.1%: nuclear
13.0%: mitochondrial
4.3%: vesicles of secretory system

>> prediction for CG55906-04 is cyt (k = 23)

A search of the NOV32a protein against the Geneseq database, a proprietary database that contains sequences published in patents and patent publication, yielded several homologous proteins shown in Table 32D.

TABLE 32D
Geneseq Results for NOV32a

| | | NOV32a | Identities/ | | |
|---|---|---|---|---|---|
| | | Residues/ | Similarities for | | |
| Geneseq | Protein/Organism/Length | Match | the Matched | | Expect |
| Identifier | [Patent #, Date] | Residues | Region | | Value |
| AAO21705 | Human secreted protein SEQ ID No | 1 . . . 1372 | 1297/1372 | (94%) | 0.0 |
| | 47 - Homo sapiens, 1325 aa. | 1 . . . 1325 | 1306/1372 | (94%) | |
| | [WO200238602-A2, 16-MAY-2002] | | | | |
| ABB12254 | Human S3-12 homologue, SEQ ID | 1 . . . 1235 | 1234/1235 | (99%) | 0.0 |
| | NO: 2624 - Homo sapiens, 1250 aa. | 17 . . . 1250 | 1234/1235 | (99%) | |
| | [WO200157188-A2, 09-AUG-2001] | | | | |
| ABP53597 | Human NOV19 protein SEQ ID | 1 . . . 1074 | 1026/1080 | (95%) | 0.0 |
| | NO: 58 - Homo sapiens, 1225 aa. | 1 . . . 1033 | 1026/1080 | (95%) | |
| | [WO200262999-A2, 15-AUG-2002] | | | | |
| AAY95851 | Autoantigen diagnostic of | 314 . . . 749 | 431/436 | (98%) | 0.0 |
| | endometriosis - Homo sapiens, 439 | 4 . . . 439 | 432/436 | (98%) | |
| | aa. [WO200047739-A2, 17-AUG-2000] | | | | |
| AAY48492 | Human breast tumour-associated | 1049 . . . 1372 | 323/324 | (99%) | 0.0 |
| | protein 37 - Homo sapiens, 324 aa. | 1 . . . 324 | 323/324 | (99%) | |
| | [DE19813835-A1, 23-SEP-1999] | | | | |

In a BLAST search of public sequence databases, the NOV32a protein was found to have homology to the proteins shown in the BLASTP data in Table 32E.

TABLE 32E
Public BLASTP Results for NOV32a

| | | NOV32a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| Q96Q06 | Hypothetical protein KIAA1881 - | 91 . . . 1372 | 1256/1348 | (93%) | 0.0 |
| | Homo sapiens (Human), 1348 aa | 1 . . . 1348 | 1264/1348 | (93%) | |
| | (fragment). | | | | |
| O88492 | S3-12 - Mus musculus (Mouse), | 1 . . . 1370 | 843/1432 | (58%) | 0.0 |
| | 1403 aa. | 1 . . . 1401 | 1043/1432 | (71%) | |
| Q98MG7 | Hypothetical glycine-rich protein | 141 . . . 1141 | 352/1088 | (32%) | 5e−71 |
| | mlr0587 - Rhizobium loti | 1078 . . . 2144 | 387/1088 | (35%) | |
| | (Mesorhizobium loti), 3145 aa. | | | | |
| Q98MG8 | Hypothetical glycine-rich protein | 128 . . . 1141 | 325/1070 | (30%) | 8e−63 |
| | mlr0585 - Rhizobium loti | 329 . . . 1379 | 384/1070 | (35%) | |
| | (Mesorhizobium loti), 2147 aa. | | | | |
| Q96WU8 | Hypothetical 119.8 kDa protein - | 140 . . . 1088 | 265/996 | (26%) | 4e−42 |
| | Schizosaccharomyces pombe | 266 . . . 1173 | 348/996 | (34%) | |
| | (Fission yeast), 1195 aa (fragment). | | | | |

PFam analysis predicts that the NOV32a protein contains the domains shown in the Table 32F.

TABLE 32F
Domain Analysis of NOV32a

| | | | |
|---|---|---|---|
| | | Identities/ | |
| Pfam | NOV32a | Similarities for | Expect |
| Domain | Match Region | the Matched Region | Value |

| | | | | |
|---|---|---|---|---|
| LEA | 87 . . . 156 | 16/75 | (21%) | 0.00076 |
| | | 44/75 | (59%) | |
| LEA | 516 . . . 589 | 15/76 | (20%) | 0.96 |
| | | 46/76 | (61%) | |
| perilipin | 592 . . . 1356 | 89/788 | (11%) | 0.045 |
| | | 435/788 | (55%) | |

### Example 33

The NOV33 clone was analyzed, and the nucleotide and encoded polypeptide sequences are shown in Table 33A.

TABLE 33A

| | | |
|---|---|---|
| NOV33a,CG55908-01 | SEQ ID NO:813 | 840 bp |
| DNA Sequence | ORF Start: at 2 | ORF Stop: TAA at 836 |

GGCCTCCGGGATTTGCTACCTTTTTGGCTCCCTGCTCGTCGAACTGCTCTTCTCACGGGCTGTCGCCT
TCAATCTGGACGTGATGGGTGCCTTGCGCAAGGAGGGCGAGCCAGGCAGCCTCTTCGGCTTCTCTGTG
GCCCTGCACCGGCAGTTGCAGCCCCGACCCCAGAGCTGGCTGCTGGTGGGTGCTCCCCAGGCCCTGGC
TCTTCCTGGGCAGCAGGCGAATCGCACTGGAGGCCTCTTCGCTTGCCCGTTGAGCCTGGAGGAGACTG
ACTGCTACAGAGTGGACATCGACCAGGGAGCTGATATGCAAAAGGAAAGCAAGGAGAACCAGTGGTTG
GGAGTCAGTGTTCGGAGCCAGGGGCCTGGGGGCAAGATTGTTGACTGCGCCCGGGGCACGGCCAACTG
TGTGGTGTTCAGCTGCCCACTCTACAGCTTTGACCGCGCGGCTGTGCTGCATGTCTGGGGCCGTCTCT
GGAACAGCACCTTTCTGGAGGAGTACTCAGCTGTGAAGTCCCTGGAAGTGATTGTCCGGGCCAACATC
ACAGTGAAGTCCTCCATAAAGAACTTGATGCTCCGAGATGCCTCCACAGTGATCCCAGTGATGGTATA
CTTGGACCCCATGGCTGTGGTGGCAGAAGGAGTGCCCTGGTGGGTCATCCTCCTGGCTGTACTGGCTG
GGCTGCTGGTGCTAGCACTGCTGGTGCTGCTCCTGTGGAAGTGTGGCTTCTTCCATCGGAGCAGCCAG
AGCTCATCTTTTCCCACCAACTATCACCGGGCCTGTCTGGCTGTGCAGCCTTCAGCCATGGAAGTTGG
GGGTCCAGGGACTGTGGGGTAACT

| | | | |
|---|---|---|---|
| NOV33a, CG55908-01 | SEQ ID NO:814 | 278 aa | MW at 30129.7 kD |
| Protein Sequence | | | |

ASGTCYLFGSLLVELLFSRAVAFNLDVMGALRKEGEPGSLFGFSVALHRQLQPRPQSWLLVGAPQALA
LPGQQANRTGGLFACPLSLEETDCYRVDIDQGADMQKESKENQWLGVSVRSQGPGGKIVDCARGTANC
VVFSCPLYSFDPAAVLHVWGRLWNSTFLEEYSAVKSLEVIVRANITVKSSIKNLMLRDASTVIPVMVY
LDPMAVVAEGVPWWVILLAVLAGLLVLALLVLLLWKCGFFHRSSQSSSFPTNYHRACLAVQPSAMEVG
GPGTVG

| | | |
|---|---|---|
| NOV33b, 253116407 | SEQ ID NO:815 | 780 bp |
| DNA Sequence | ORF Start: at 1 | ORE Stop: end of sequence |

GGATCCTTCAATCTGGACGTGATGGGTGCCTTGCGCAAGGAGGGCGAGCCAGGCAGCCTCTTCGGCTT
CTCTGTGGCCCTGCACCGGCAGTTGCAGCCCCGACCCCAGAGCTGGCTGCTGGTGGGTGCTCCCCAGG
CCCTGGCTCTTCCTGGGCAGCAGGCGAATCGCACTGGAGGCCTCTTCGCTTGCCCGTTGAGCCTGGAG
GAGACTGACTGCTACAGAGTGGACATCGACCAGGGAGCTGATATGCAAAAGGAAAGCAAGGAGAACCA
GTGGTTGGGAGTCAGTGTTCGGAGCCAGGGGCCTGGGGGCAAGATTGTTGACTGCGCCCGGGGCACGG
CCAACTGTGTGGTGTTCAGCTGCCCACTCTACAGCTTTGACCGCGCGGCTGTGCTGCATGTCTGGGGC
CGTCTCTGGAACAGCACCTTTCTGGAGGAGTACTCAGCTGTGAAGTCCCTGGAAGTGATTGTCCGGGC
CAACATCACAGTGAAGTCCTCCATAAAGAACTTGATGCTCCGAGATGCCTCCACAGTGATCCCAGTGA
TGGTATACTTGGACCCCATGGCTGTGGTGGCAGAAGGAGTGCCCTGGTGGGTCATCCTCCTGGCTGTA
CTGGCTGGGCTGCTGGTGCTAGCACTGCTGGTGCTGCTCCTGTGGAAGTGTGGCTTCTTCCATCGGAG
CAGCCAGAGCTCATCTTTTCCCACCAACTATCACCGGGCCTGTCTGGCTGTGCAGCCTTCAGCCATGG
AAGTTGGGGGTCCAGGGACTGTGGGGCTCGAG

| | | | |
|---|---|---|---|
| NOV33b, 253116407 | SEQ ID NO:816 | 260 aa | MW at 28204.4 kD |
| Protein Sequence | | | |

GSFNLDVMGALRKEGEPGSLFGFSVALHRQLQPRPQSWLLVGAPQALALPGQQANRTGGLFACPLSLE
ETDCYRVDIDQGADMQKESKENQWLGVSVRSQGPGGKIVDCARGTANCVVFSCPLYSFDRAAVLHVWG
RLWNSTFLEEYSAVKSLEVIVRANITVKSSIKNLMLRDASTVIPVMVYLDPMAVVAEGVPWWVILLAV
LAGLLVLALLVLLLWKCGFFHRSSQSSSFPTNYHRACLAVQPSAMEVGGPGTVGLE

| | | |
|---|---|---|
| NOV33c, 253116412 | SEQ ID NO:817 | 567 bp |
| DNA Sequence | ORF Start: at 1 | ORF Stop: end of sequence |

GGATCCTTCAATCTGGACGTGATGGGTGCCTTGCGCAAGGAGGGCGAGCCAGGCAGCCTCTTCGGCTT
CTCTGTGGCCCTGCACCGGCAGTTGCAGCCCCGACCCCAGAGCTGGCTGCTGGTGGGTGCTCCCCAGG
CCCTGGCTCTTCCTGGGCAGCAGGCGAATCGCACTGGAGGCCTCTTCGCTTGCCCGTTGAGCCTGGAG
GAGACTGACTGCTACAGAGTGGACATCGACCAGGGAGCTGATATGCAAAAGGAAAGCAAGGAGAACCA
GTGGTTGGGAGTCAGTGTTCGGAGCCAGGGGCCTGGGGGCAAGATTGTTGACTGCGCCCGGGGCACGG
CCAACTGTGTGGTGTTCAGCTGCCCACTCTACAGCTTTGACCGCGCGGCTGTGCTGCATGTCTGGGGC
CGTCTCTGGAACAGCACCTTTCTGGAGGAGTACTCAGCTGTGAAGTCCCTGGAAGTGATTGTCCGGGC
CAACATCACAGTGAAGTCCTCCATAAAGAACTTGATGCTCCGAGATGCCTCCACAGTGATCCCAGTGA
TGGTATACTTGGACCCCCTCGAG

| | | | |
|---|---|---|---|
| NOV33c, 253116412 | SEQ ID NO:818 | 189 aa | MW at 20685.5 kD |
| Protein Sequence | | | |

GSFNLDVMGALRKEGEPGSLFGFSVALHRQLQPRPQSWLLVGAPQALALPGQQANRTGGLFACPLSLE
ETDCYRVDIDQGADMQKESKENQWLGVSVRSQGPGGKIVDCARGTANCVVFSCPLYSFDRAAVLHVWG
RLWNSTFLEEYSAVKSLEVIVRANTTVKSSIKNLMLRDASTVIPVMVYLDPLE

A ClustalW comparison of the above protein sequences yields the following sequence alignment shown in Table 33B.

TABLE 33B
Comparison of the NOV33 protein sequences.

| | | |
|---|---|---|
| NOV33a | ASGICYLFGSLLVELLFSRAVAFNLDVMGALRKEGEPGSLFGFSVALHRQLQPRPQSWLL | (SEQ ID NO:814) |
| NOV33b | --------------------GSFNLDVMGALRKEGEPGSLFGFSVALHRQLQPRPQSWLL | (SEQ ID NO:816) |
| NOV33c | --------------------GSFNLDVMGALRKEGEPGSLFGFSVALHRQLQPRPQSWLL | (SEQ ID NO:818) |
| NOV33a | VGAPQALALPGQQANRTGGLFACPLSLEETDCYRVDIDQGADMQKESKENQWLGVSVRSQ | |
| NOV33b | VGAPQALALPGQQANRTGGLFACPLSLEETDCYRVDIDQGADMQKESKENQWLGVSVRSQ | |
| NOV33c | VGAPQALALPGQQANRTGGLFACPLSLEETDCYRVDIDQGADMQKESKENQWLGVSVRSQ | |
| NOV33a | GPGGKIVDCARGTANCVVFSCPLYSFDRAAVLHVWGRLWNSTFLEEYSAVKSLEVIVRAN | |
| NOV33b | GPGGKIVDCARGTANCVVFSCPLYSFDRAAVUHVWGRLWNSTFLEEYSAVKSLEVIVRAN | |
| NOV33c | GPGGKIVDCARGTANCVVFSCPLYSFDRAAVLHVWGRLWNSTFLEEYSAVKSLEVIVRAN | |
| NOV33a | ITVKSSIKNLMLRDASTVIPVMVYLDPMAVVAEGVPWWVTLLAVLAGLLVLAILVLLLWK | |
| NOV33b | ITVKSSIKNLMLRDASTVIPVMVYLDPMAVVAEGVPWWVTLLAVLAGLLVLALLVLLLWK | |
| NOV33c | ITVKSSIKNLMLRDASTVIPVMVYLDPLE------------------------------- | |
| NOV33a | CGFFHRSSQSSSFPTNYHRACLAVQPSAMEVGGPGTVG-- | |
| NOV33b | CGFFHRSSQSSSFPTNYHRACLAVQPSAMEVGGPGTVGLE | |
| NOV33c | ---------------------------------------- | |

Further analysis of the NOV33a protein yielded the following properties shown in Table 33C.

TABLE 33C
Protein Sequence Properties NOV33a

| | |
|---|---|
| SignalP analysis: | Cleavage site between residues 23 and 24 |
| PSORT II analysis: | |

| PSG: | a new signal peptide prediction method | |
|---|---|---|
| | N-region: | length 0; pos. chg 0; neg. chg 0 |
| | H-region: | length 13; peak value 8.16 |
| | PSG score: | 3.76 |

| | |
|---|---|
| GvH: | von Heijne's method for signal seq. recognition |
| | GvH score (threshold: −2.1): −0.41 |
| | possible cleavage site: between 22 and 23 |

>>> Seems to have a cleavable signal peptide (1 to 22)

| ALOM: | Klein et al's method for TM region allocation | | |
|---|---|---|---|
| | Init position for calculation: 23 | | |
| | Tentative number of TMS(s) for the threshold 0.5: 2 | | |
| | Number of TMS(s) for threshold 0.5: 1 | | |
| | INTEGRAL | Likelihood = | Transmembrane 219-235 |
| | | −15.81 | |

| | |
|---|---|
| PERIPHERAL | Likelihood = 2.65 (at 136) |

| | |
|---|---|
| | ALOM score: −15.81 (number of TMSs: 1) |
| MTOP: | Prediction of membrane topology (Hartmann et al.) |
| | Center position for calculation: 11 |
| | Charge difference: −2.0 C(−1.0)-N( 1.0) |
| | N >= C: N-terminal side will be inside |

>>> membrane topology: type 1a (cytoplasmic tail 236 to 278)

| MITDISC: | discrimination of mitochondrial targeting seq | | | |
|---|---|---|---|---|
| | R content: | 1 | Hyd Moment(75): | 2.41 |
| | Hyd Moment(95): | 4.01 | G content: | 2 |
| | D/E content: | 2 | S/T content: | 3 |

| | |
|---|---|
| | Score: −6.94 |
| Gavel: | prediction of cleavage sites for mitochondrial preseq |
| | cleavage site motif not found |

NUCDISC: discrimination of nuclear localization signals

| | |
|---|---|
| | pat4: none |
| | pat7: none |
| | bipartite: none |
| | content of basic residues: 7.9% |
| | NLS Score: −0.471 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: 256
Dileucine motif in the tail: found

LL at 236

checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| | total: 0 residues |

Final Results (k = 9/23):

44.4%: endoplasmic reticulum
22.2%: Golgi
11.1%: plasma membrane
11.1%: vesicles of secretory system
11.1%: extracellular, including cell wall

>> prediction for CG55908-01 is end (k = 9)

A search of the NOV33a protein against the Geneseq database, a proprietary database that contains sequences published in patents and patent publication, yielded several homologous proteins shown in Table 33D.

TABLE 33D
Geneseq Results for NOV33a

| | | NOV33a | Identities/ | | |
|---|---|---|---|---|---|
| | | Residues/ | Similarities for | | |
| Geneseq | Protein/Organism/Length | Match | the Matched | | Expect |
| Identifier | [Patent #, Date] | Residues | Region | | Value |
| ABG69620 | Human NOV13 protein - Homo | 1 . . . 278 | 278/278 | (100%) | e−161 |
| | sapiens, 278 aa. [WO200250277-A2, | 1 . . . 278 | 278/278 | (100%) | |
| | 27-JUN-2002] | | | | |
| ABG61788 | Novel ITGA7 precursor-like receptor | 58 . . . 278 | 167/230 | (72%) | 3e−84 |
| | protein #1 - Homo sapiens, 1143 aa. | 917 . . . 1143 | 180/230 | (77%) | |
| | [WO200229058-A2, 11-APR-2002] | | | | |
| ABG61789 | Novel ITGA7 precursor-like receptor | 128 . . . 278 | 150/151 | (99%) | 4e−83 |
| | protein #2 - Homo sapiens, 1035 aa. | 885 . . . 1035 | 151/151 | (99%) | |
| | [WO200229058-A2, 11-APR-2002] | | | | |
| ABU58459 | Human PRO polypeptide #60 - Homo | 1 . . . 130 | 129/130 | (99%) | 3e−70 |
| | sapiens, 1141 aa. [US2003027272- | 12 . . . 141 | 129/130 | (99%) | |
| | A1, 06-FEB-2003] | | | | |
| ABU56990 | Human PRO polypeptide #60 - Homo | 1 . . . 130 | 129/130 | (99%) | 3e−70 |
| | sapiens, 1141 aa. [US2003027280- | 12 . . . 141 | 129/130 | (99%) | |
| | A1, 06-FEB-2003] | | | | |

In a BLAST search of public sequence databases, the NOV33a protein was found to have homology to the proteins shown in the BLASTP data in Table 33E.

TABLE 33E
Public BLASTP Results for NOV33a

| | | NOV33a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAD48689 | Sequence 41 from Patent | 58 . . . 278 | 167/230 (72%) | 1e−83 |
| | WO0229058 - Homo sapiens | 917 . . . 1143 | 180/230 (77%) | |
| | (Human), 1143 aa. | | | |
| JC5950 | integrin alpha-7 chain precursor - | 1 . . . 130 | 129/130 (99%) | 9e−70 |
| | human, 1137 aa. | 12 . . . 141 | 129/130 (99%) | |
| AAH50280 | Integrin, alpha 7 - Homo sapiens | 1 . . . 130 | 129/130 (99%) | 9e−70 |
| | (Human), 1137 aa. | 12 . . . 141 | 129/130 (99%) | |
| Q13683 | Integrin alpha-7 precursor - Homo | 1 . . . 130 | 129/130 (99%) | 9e−70 |
| | sapiens (Human), 1181 aa. | 12 . . . 141 | 129/130 (99%) | |
| S40148 | integrin alpha-7A chain - rat, 148 | 150 . . . 278 | 118/129 (91%) | 1e−61 |
| | aa. | 1 . . . 129 | 122/129 (94%) | |

PFam analysis predicts that the NOV33a protein contains the domains shown in the Table 33F.

TABLE 33F
Domain Analysis of NOV33a

| | | | |
|---|---|---|---|
| Pfam | NOV33a | Identities/ | Expect |
| Domain | Match Region | Similarities for | Value |
| | | the Matched Region | |

### Example 34

The NOV34 clone was analyzed, and the nucleotide and encoded polypeptide sequences are shown in Table 34A.

TABLE 34A
NOV34 Sequence Analysis

| | | |
|---|---|---|
| NOV34a, CG56077-06 | SEQ ID NO:819 | 2106 bp |
| DNA Sequence | ORF Start: at 1 | ORF Stop: TGA at 1636 |

TCCCGAGACTTTGGAAGTTCTCAGCTATTACTTTATTACATAGGATTTCTGTGTCTTTTCTCATCTCT
TTTCCTTTTGGAAAGACCCCCAAAGGGAAAGTGTCCCCTGCGCTGCTCCTGCTCTAAAGACAGCGCCC
TGTGTGAGGGCTCCCCGGACCTGCCCGTCAGCTTCTCTCCGACCCTGCTGTCACTGTCACTCGTCAGG
ACGGGAGTCACCCAGCTGAAGGCCGGCAGCTTCCTGAGAATTCCGTCTCTGCACCTGCTGCTCTTCAC
CTCCAACTCCTTCTCCGTGATTGAGGACGATGCATTTGCGGGCCTGTCCCACCTGCAGTACCTGTTCA
TCGAGGACAATGAGATTGGCTCCATCTCTAAGAATGCCCTCAGAGGACTTCGCTCGCTTACACACCTG
AGCCTGGCCAATAACCATCTGGAGACCCTCCCCAGATTCCTGTTCCGAGGCCTGGACACCCTTACTCA
TGTGGACCTCCGCGGGAACCCGTTCCAGTGTGACTGCCGCGTCCTCTGGCTCCTGCAGTGGATGCCCA
CCGTGAATGCCAGCGTGGGGACCGGCGCCTGTGCGGGCCCCGCCTCCCTGAGCCACATGCAGCTCCAC
CACCTCGACCCCAAGACTTTCAAGTGCCCACCCACAGAGCTGTCCTGGTTCCAGACGGTGGGGGAGTC
GGCACTGAGCGTAGAGCCCTTCTCCTACCAAGGGGAGCCTCACATTGTGCTGGCACAGCCCTTCGCCG
GCCGCTGCCTGATTCTCTCCTGGGACTACAGCCTGCAGCGCTTCCGGCCCGAGGAAGAGCTGCCCACA
GCGGCCTCCGTGGTGTCCTGCAAGCCACTGGTGCTGGGCCCGAGCCTCTTCGTGCTGGCTGCCCGCCT
GTGGGGGGGCTCACAGCTGTGGGCCCGGCCCAGTCCCGGCCTGCGCCTGGCCCCAACGCAGACCCTGG
CCCCGCGGCGGCTGCTGCGGCCCAATGACGCCGAGCTCCTGTGGCTGGAAGGGCAACCCTGCTTCGTG
GTGGCCGATGCCTCCAAGGCGGGCAGCACCACGCTGCTGTGCCGCGACGGGCCCGGCTTTTACCCGCA
CCAGAGCCTGCACGCCTGGCACCGGGACACGGACGCTGAGGCCCTGGAGCTGGACGGCCGGCCCCACC
TGCTGCTGGCCTCGGCTTCCCAGCGGCCCGTGCTCTTCCACTGGACCGGTGGCCGCTTCGAGAGACGC
ACGGACATCCCCGAGGCCGAGGATGTCTATGCCACACGCCACTTCCAGGCTGGTGGGGACGTGTTCCT
GTGCCTCACACGCTACATTGGGGACTCCATGGTGATGCGCTGGGACGGCTCCATGTTTCGTCTGCTGC
AGCAACTTCCCTCGCGCGGTGCCCACGTCTTCCAGCCACTGCTCATCGCCAGGGACCAGTTGGCCATC
CTAGGCAGCGACTTCGCCTTCAGCCAGGTCCTCCGCCTTGAGCCTGACAAGGGGCTCCTGGAGCCACT
GCAGGAGCTGGGGCCTCCGGCCCTGGTGGCCCCCCGTGCCTTTGCCCACATCACTATGGCCGGCAGAC
GCTTCCTCTTTGCTGCTTGCTTTAAGGGCCCCACACAGATCTACCAGCATCACGAGATCGACCTCAGT
GCCTGAGACCACCAACGGGACTCTGGGCATGGCTGGGGCCCCTGGACGGCCCCTTGGCTGGCTCCTGG
CCCTACTTGGGGTGATGGTCCGCCTGTGAGCTGCTGACCGTGGGCCACGTTCATCAGCCACACGTCTA
GGCCTTAAGCCCACTTCTTAAAGGATCTGCACCCATGGGGGGACATAGAGGGTCCCAGCCTACTGGAC
CTCCCGAGCTGCCCTTCAGGTCTAAAGCAACATCTGGACATCTCGGCTGGGGCAGATTCCCCATCAAA
GGCAACCTGGGGGGTGCAGGCTGGTGCACTGCATGGAGTCGAGGTGGAGGTCACGTGCAGAGCTCCAT
GAGGTCCCGGAGCCCCCCTCTCAACCCGCTCCTACCCCGCTGGTCCCTCTGAAAGCACCGAGGGGTTG
ATGGCATTCCTTTCCCATTCTAAACTCCACGCAGCCAATCGCTTTCCTGGAGGAGCGCCAACCTCG

| | | | |
|---|---|---|---|
| NOV34a, CG56077-06 | SEQ ID NO:820 | 545 aa | MW at 60447.7 kD |
| Protein Sequence | | | |

SRDFGSSQLLLYYIGFLCLFSSLFLLERPPKGKCPLRCSCSKDSALCEGSPDLPVSFSPTLLSLSLVR
TGVTQLKAGSFLRIPSLHLLLFTSNSFSVIEDDAFAGLSHLQYLFIEDNEIGSISKNALRGLRSLTHL
SLANNHLETLPRFLFRGLDTLTHVDLRGNPFQCDCRVLWLLQWMPTVNASVGTGACAGPASLSHMQLH
HLDPKTFKCPPTELSWFQTVGESALSVEPFSYQGEPHIVLAQPFAGRCLILSWDYSLQRFRPEEELPT
AASVVSCKPLVLGPSLFVLAARLWGGSQLWARPSPGLRLAPTQTLAPRRLLRPNDAELLWLEGQPCFV
VADASKAGSTTLLCRDGPGFYPHQSLHAWHRDTDAEALELDGRPHLLLASASQRPVLFHWTGGRFERR
TDIPEAEDVYATRHFQAGGDVFLCLTRYIGDSMVMRWDGSMFRLLQQLPSRGAHVFQPLLIARDQLAI
LGSDFAFSQVLRLEPDKGLLEPLQELGPPALVAPRAFAHITMAGRRFLFAACFKGPTQIYQ
HHEIDLSA

| | | |
|---|---|---|
| NOV34b, CG56077-01 | SEQ ID NO:821 | 1859 bp |
| DNA Sequence | ORF Start: ATG at 101 | ORF Stop: TGA at 1760 |

GTAACTCCTCTTCATCCACTGGGGAGGAAGGTGAGGCAGGCGGGCCCAATTCATTCGCCTCCGGTACT
TGCAAGCCTCGCTCAGTCTTAAGCAAGAGGGGATGGATTCGCCCGCAGCACTGAGAATCCAGGGGCAG
GCGGGATGGCGTTCAGGCGCTGTTGCTAGAAATCTCTGTCTTTACTCTGTTTTGGTCATTACGGAGGG
AAGACAGCCCCCAAAGGGAAAGTGTCCCCTGCGCTGCTCCTGCTCTAAAGACAGCGCCCTGTGTGAGG
GCTCCCCGGACCTGCCCGTCAGCTTCTCTCCGACCCTGCTGTCACTCTCACTCGTCAGGACGGGAGTC
ACCCAGCTGAAGGCCGGCAGCTTCCTGAGAATTCCGTCTCTGCACCTGCTCCTCTTCACCTCCAACTC
CTTCTCCGTGATTGAGGACGATGCATTTGCGGGCCTGTCCCACCTGCAGTACCTCTTCATCGAGGACA
ATGAGATTGGCTCCATCTCTAAGAATGCCCTCAGAGGACTTCGCTCGCTTACACACCTAAGCCTGGCC
AATAACCATCTGGAGACCCTCCCCAGATTCCTGTTCCGAGGCCTGGACACCCTTACTCATGTGGACCT
CCGCGGGAACCCGTTCCAGTGTGACTGCCGCGTCCTCTGGCTCCTGCAGTGGATGCCCACCGTGAATG
CCAGCGTGGGGACCGGCGCCTGTGCGGGCCCCGCCTCCCTGAGCCACATGCAGCTCCACCACCTCGAC
CCCAAGACTTTCAAGTGCAGAGCCATAGAGCTGTCCTGGTTCCAGACGGTGGGGGAGTCGGCACTGAG
CGTAGAGCCCTTCTCCTACCAAGGGGAGCCTCACATTGTGCTGGCACAGCCCTTCGCCGGCCGCTGCC
TGATTCTCTCCTGGGACTACAGCCTGCAGCGCTTCCGGCCCGAGGAAGAGCTGCCCGCGGCCTCCGTG
GTGTCCTGCAAGCCACTGGTGCTGGGCCCGAGCCTCTTCGTGCTGGCTGCCCGCCTGTGGGGGGGCTC
ACAGCTGTGGGCCCGGCCCAGTCCCGGCCTGCGCCTGGCCCCAACGCAGACCCTGGCCCCGCGGCGGC
TGCTGCGGCCCAATGACGCCGAGCTCCTGTGGCTGGAAGGGCAACCCTGCTTCGTGGTGGCCGATGCC
TCCAAGGCGGGCAGCACCACGCTGCTGTGCCGCGACGGGCCCGGCTTTTACCCGCACCAGAGCCTGCA
CGCCTGGCACCGGGACACGGACGCTGAGGCCCTGGAGCTGGACGGCCGGCCCCACCTGCTGCTGGCCT
CGGCTTCCCAGCGGCCCGTGCTCTTCCACTGGACCGGTGGCCGCTTCGAGAGACGCACAGACATCCCG
AGGGCCGAGGATGTCTATGCCACACGCCACTTCCAGGCTGGTGGGGACGTGTTCCTGTGCCTCACACG
CTACATTGGGGACTCCATGGTCATGCGCTGGGACGGCTCCATGTTTCGTCTGCTGCAGCAACTTCCCT
CGCGCGGTGCCCACGTCTTCCAGCCACTGCTCATCGCCAGGGACCAGCTGGCCATCCTAGGCAGCGAC
TTCGCCTTCAGCCAGGTCCTCCGCCTTGAGCCTGACAAGGGGCTCCTGGAGCCACTGCAGGAGCTGGG
GCCTCCGGCCCTGGTGGCCCCCCGTGCCTTTGCCCACATCACTATGGCCGGCAGACGCTTCCTCTTTG
CTGCTTGCTTTAAGGGCCCCACACAGATCTACCAGCATCACGAGATCGACCTCAGTGCCTGAGACCAC
CAACGGGACTCTGGGCATGGCTGGGGCCCCTGGACGGCCCCTTGGCTGGCTCCTGGCCCTACTTGGGG
TGATGGCCCGCCTGTGAGCTGCT

| | | | |
|---|---|---|---|
| NOV34b, CG56077-01 | SEQ ID NO:822 | 553 aa | MW at 61118.5 kD |
| Protein Sequence | | | |

MDSPAALRIQGQAGWRSGAVARNLCLYSVLVITEGRQPPKGKCPLRCSCSKDSALCEGSPDLPVSFSP
TLLSLSLVRTGVTQLKAGSFLRIPSLHLLLFTSNSFSVIEDDAFAGLSHLQYLFIEDNEIGSISKNAL
RGLRSLTHLSLANNHLETLPRFLFRGLDTLTNVDLRGNPFQCDCRVLWLLQWMPTVNASVGTGACAGP
ASLSHMQLHHLDPKTFKCRATELSWFQTVGESALSVEPFSYQGEPHIVLAQPFAGRCLILSWDYSLQR
FRPEEELPAASVVSCKPLVLGPSLFVLAARLWGGSQLWARPSPGLRLAPTQTLAPRRLLRPNDAELLW
LEGQPCFVVADASKAGSTTLLCRDGPGFYPHQSLHAWHRDTDAEALELDGRPHLLLASASQRPVLFHW
TGGRFERRTDIPRAEDVYATRHFQAGGDVFLCLTRYIGDSMVMRWDGSMFRLLQQLPSRGAHVFQPLL
IARDQLAILGSDFAFSQVLRLEPDKGLLEPLQELGPPALVAPRAFAHITMAGRRFLFAACFKGPTQIY
QHHEIDLSA

| | | |
|---|---|---|
| NOV34c, CG56077-02 | SEQ ID NO:823 | 1482 bp |
| DNA Sequence | ORF Start: at 1 | ORF Stop: TGA at 1480 |

TCCCGAGACTTTGGAAGTTCTCAGCTATTACTTTATTACATAGGATTTCTGTGTCTTTTCTCATCTCT
TTTCCTTTTGGAAATTGGAAGACCCCCAAAGGGAAAGTGTCCCCTGCGCTGCTCCTGCTCTAAAGACA
GCGCCCTGTGTGAGGGCTCCCCGGACCTGCCCGTCAGCTTCTCTCCGACCCTGCTGTCACTGACTGCC
CACATCCCCAGCTCACTCGTCAGGACGGGAGTCACCCAGCTGAAGGCCGGCAGCTTCCTGAGAATTCC
GTCTCTGCACCTGCTGCTCTTCACCTCCAACTCCTTCTCCGTGATTGAGGACGATGCATTTGCGGGCC
TGTCCCACCTGCAGTACCTGTTCATCGAGGACAATGAGATTGGCTCCATCTCTAAGAATGCCCTCAGA
GGACTTCGCTCGCTTACACACCTGAGCCTGGCCAATAACCATCTGGAGACCCTCCCCAGATTCCTGTT
CCGAGGCCTGGACACCCTTACTCATGTGGACCTCCGCGGGAACCCGTTCCAGTGTGACTGCCGCGTCC
TCTGGCTCCTGCAGTGGATGCCCACCGTGAATGCCAGCGTGGGGACCGGCGCCTGTGCGGGCCCCGCC
TCCCTGAGCCACATGCAGCTCCACCACCTCGACCCCAAGACTTTCAAGTGCACAGCGGCCTCCGTGGT
GTCCTGCAAGCCACTGGTGCTGGGCCCGAGCCTCTTCGTGCTGGCTGCCCGCCTGTGGGGGGGCTCAC
AGCTGTGGGCCCGGCCCAGTCCCGGCCTGCGCCTGGCCCCAACGCAGACCCTGGCCCCGCGGCGGCTG
CTGCGGCCCAATGACGCCGAGCTCCTGTGGCTGGAAGGGCAACCCTGCTTCGTGGTGGCCGATGCCTC
CAAGGCGGGCAGCACCACGTGCAGCGCTTCCGGCCCGAGGAAGAGCTGCCCGAGCCTGCACGCCTGGC
ACCGGGACACGGACGCTGAGGCCCTGGAGCTGGACGGCCGGCCCCACCTGCTGCTGGCCTCGGCTTCC
CAGCGGCCCGTGCTCTTCCACTGGACCGGTGGCCGCTTCGAGAGACGCACGGACATCCCCGAGGCCGA
GGATGTCTATGCCACACGCCACTTCCAGGCTGGTGGGGACGTGTTCCTGTGCCTCACACGCTACATTG
GGGACTCCATGGTCATGCGCTGGGACGGCTCCATGTTTCGTCTGCTGCAGCAACTTCCCTCGCGCGGT
GCCCACGTCTTCCAGCCACTGCTCATCGCCAGGGACCAATTGGCCATCCTAGGCAGCGACTTCGCCTT
CAGCCAGGTCCTCCGCCTTGAGCCTGACAAGGGGCTCCTGGAGCCACTGCAGGAGCTGGGGCCTCTGG
CCCTGGTGGCCCCCCGTGCCTTTGCCCACATCACTATGGCCGGCAGACGCTTCCTCTTTGCTGCTTGC
TTTAAGGGCCCCACACAGATCTACCAGCATCACGAGATCGACCTCAGTGCCTGA

| | | | |
|---|---|---|---|
| NOV34c, CG56077-02 | SEQ ID NO:824 | 493 aa | MW at 54197.8 kD |
| Protein Sequence | | | |

SRDFGSSQLLLYYIGFLCLFSSLFLLEIGRPPKGKCPLRCSCSKDSALCEGSPDLPVSFSPTLLSLTA
HIPSSLVRTGVTQLKAGSFLRIPSLHLLLFTSNSFSVIEDDAFAGLSHLQYLFIEDNEIGSISKNALR
GLRSLTHLSLANNHLETLPRFLFRGLDTLTHVDLRGNPFQCDCRVLWLLQWMPTVNASVGTGACAGPA
SLSHMQLHHLDPKTFKCTAASVVSCKPLVLGPSLFVLAARLWGGSQLWARPSPGLRLAPTQTLAPRRL
LRPNDAELLWLEGQPCFVVADASKAGSTTCSASGPRKSCPSLHAWHRDTDAEALELDGRPHLLLASAS
QRPVLFHWTGGRFERRTDIPEAEDVYATRHFQAGGDVFLCLTRYIGDSMVMRWDGSMFRLLQQLPSRG
AHVFQPLLIARDQLAILGSDFAFSQVLRLEPDKGLLEPLQELGPLALVAPRAFAHITMAGRRFLFAAC
FKGPTQIYQHHEIDLSA

| | | |
|---|---|---|
| NOV34d, CG56077-03 | SEQ ID NO:825 | 741 bp |
| DNA Sequence | ORF Start: at 1 | ORF Stop: end of sequence |

ACGGAGGGAAGACAGCCCCCAAAGGGAAAGTGTCCCCTGCGCTGCTCCTGCTCTAAAGACAGCGCCCT
GTGTGAGGGCTCCCCGGACCTGCCCGTCAGCTTCTCTCCGACCCTGCTGTCACTCTCACTCGTCAGGA
CGGGAGTCACCCAGCTGAAGGCCGGCAGCTTCCTGAGAATTCCGTCTCTGCACCTGCTCCTCTTCACC
TCCAACTCCTTCTCCGTGATTGAGGACGATGCATTTGCGGGCCTGTCCCACCTGCAGTACCTCTTCAT
CGAGGACAATGAGATTGGCTCCATCTCTAAGAATGCCCTCAGAGGACTTCGCTCGCTTACACACCTAA
GCCTGGCCAATAACCATCTGGAGACCCTCCCCAGATTCCTGTTCCGAGGCCTGGACACCCTTACTCAC
GTGGACCTCCGCGGGAACCCGTTCCAGTGTGACTGCCGCGTCCTCTGGCTCCTGCAGTGGATGCCCAC
CGTGAATGCCAGCGTGGGGACCGGCGCCTGTGCGGGCCCCGCCTCCCTGAGCCACATGCAGCTCCACC
ACCTCGACCCCAAGACTTTCAAGTGCAGAGCCATAGAGCTGTCCTGGTTCCAGACGGTGGGGGAGTCG
GCACTGAGCGTAGAGCCCTTCTCCTACCAAGGGGAGCCTCACATTGTGCTGGCACAGCCCTTCGCCGG
CCGCTGCCTGATTCTCTCCTGGGACTACAGCCTGCAGCGCTTCCGGCCCGAGGAAGAGCTG

| | | | |
|---|---|---|---|
| NOV34d, CG56077-03 | SEQ ID NO:826 | 247 aa | MW at 27380.0 kD |
| Protein Sequence | | | |

TEGRQPPKGKCPLRCSCSKDSALCEGSPDLPVSFSPTLLSLSLVRTGVTQLKAGSFLRIPSLHLLLFT
SNSFSVIEDDAFAGLSHLQYLFIEDNEIGSISKNALRGLRSLTHLSLANNHLETLPRFLFRGLDTLTH
VDLRGNPFQCDCRVLWLLQWMPTVNASVGTGACAGPASLSHMQLHHLDPKTFKCRAIELSWFQTVGES
ALSVEPFSYQGEPHIVLAQPFAGRCLILSWDYSLQRFRPEEEL

| | | |
|---|---|---|
| NOV34e, CG56077-04 | SEQ ID NO:827 | 741 bp |
| DNA Sequence | ORF Start: at 1 | ORF Stop: end of sequence |

ACGGAGGGAAGACAGCCCCCAAAGGGAAAGTGTCCCCTGCGCTGCTCCTGCTCTAAAGACAGCGCCCT
CGGGAGTCACCCAGCTGAAGGCCGGCAGCTTCCTGAGAATTCCGTCTCTGCACCTGCTCCTCTTCACC
TCCAACTCCTTCTCCGTGATTGAGGACGATGCATTTGCGGGCCTGTCCCACCTGCAGTACCTCTTCAT
CGAGGACAATGAGATTGGCTCCATCTCTAAGAATGCCCTCAGAGGACTTCGCTCGCTTACACACCTAA
GCCTGGCCAATAACCATCTGGAGACCCTCCCCAGATTCCTGTTCCGAGGCCTGGACACCCTTACTCAC
GTGGACCTCCGCGGGAACCCGTTCCAGTGTGACTGCCGCGTCCTCTGGCTCCTGCAGTGGATGCCCAC
CGTGAATGCCAGCGTGGGGACCGGCGCCTGTGCGGGCCCCGCCTCCCTGAGCCACATGCAGCTCCACC
ACCTCGACCCCAAGACTTTCAAGTGCAGAGCCATAGAGCTGTCCTGGTTCCAGACGGTGGGGGAGTCG
GCACTGAGCGTAGAGCCCTTCTCCTACCAAGGGGAGCCTCACATTGTGCTGGCACAGCCCTTCGCCGG
CCGCTGCCTGATTCTCTCCTGGGACTACAGCCTGCAGCGCTTCCGGCCCGAGGAAGAGCTG

| | | | |
|---|---|---|---|
| NOV34e, CG56077-04 | SEQ ID NO:828 | 247 aa | MW at 27380.0 kD |
| Protein Sequence | | | |

TEGRQPPKGKCPLRCSCSKDSALCEGSPDLPVSFSPTLLSLSLVRTGVTQLKAGSFLRIPSLHLLLFT
SNSFSVIEDDAFAGLSHLQYLFIEDNEIGSISKNALRGLRSLTHLSLANNHLETLPRFLFRGLDTLTH
VDLRGNPFQCDCRVLWLLQWMPTVNASVGTGACAGPASLSHMQLHHLDPKTFKCRAIELSWFQTVGES
ALSVEPFSYQGEPHIVLAQPFAGRCLILSWDYSLQRFRPEEEL

| | | |
|---|---|---|
| NOV34f, CG56077-05 | SEQ ID NO:829 | 741 bp |
| DNA Sequence | ORF Start: at 1 | ORF Stop: end of sequence |

ACGGAGGGAAGACAGCCCCCAAAGGGAAAGTGTCCCCTGCGCTGCTCCTGCTCTAAAGACAGCGCCCT
GTGTGAGGGCTCCCCGGACCTGCCCGTCAGCTTCTCTCCGACCCTGCTGTCACTCTCACTCGTCAGGA
CGGGAGTCACCCAGCTGAAGGCCGGCAGCTTCCTGAGAATTCCGTCTCTGCACCTGCTCCTCTTCACC
TCCAACTCCTTCTCCGTGATTGAGGACGATGCATTTGCGGGCCTGTCCCACCTGCAGTACCTCTTCAT
CGAGGACAATGAGATTGGCTCCATCTCTAAGAATGCCCTCAGAGGACTTCGCTCGCTTACACACCTAA
GCCTGGCCAATAACCATCTGGAGACCCTCCCCAGATTCCTGTTCCGAGGCCTGGACACCCTTACTCAC
GTGGACCTCCGCGGGAACCCGTTCCAGTGTGACTGCCGCGTCCTCTGGCTCCTGCAGTGGATGCCCAC
CGTGAATGCCAGCGTGGGGACCGGCGCCTGTGCGGGCCCCGCCTCCCTGAGCCACATGCAGCTCCACC
ACCTCGACCCCAAGACTTTCAAGTGCAGAGCCATAGAGCTGTCCTGGTTCCAGACGGTGGGGGAGTCG
GCACTGAGCGTAGAGCCCTTCTCCTACCAAGGGGAGCCTCACATTGTGCTGGCACAGCCCTTCGCCGG
CCGCTGCCTGATTCTCTCCTGGGACTACAGCCTGCAGCGCTTCCGGCCCGAGGAAGAGCTG

| | | | |
|---|---|---|---|
| NOV34f, CG56077-05 | SEQ ID NO:830 | 247 aa | MW at 27380.0 kD |
| Protein Sequence | | | |

TEGRQPPKGKCPLRCSCSKDSALCEGSPDLPVSFSPTLLSLSLVRTGVTQLKAGSFLRIPSLHLLLFT
SNSFSVIEDDAFAGLSHLQYLFIEDNEIGSISKNALRGLRSLTHLSLANNHLETLPRFLFRGLDTLTH
VDLRGNPFQCDCRVLWLLQWMPTVNASVGTGACAGPASLSHMQLHHLDPKTFKCRAIELSWFQTVGES
ALSVEPFSYQGEPHIVLAQPFAGRCLILSWDYSLQRFRPEEEL

| | | |
|---|---|---|
| NOV34g, SNP13374715 of | SEQ ID NO:831 | 2106 bp |
| CG56077-06, DNA Sequence | ORF Start: at 1 | ORF Stop: TGA at 1636 |
| | SNP Pos: 1263 | SNP Change: C to T |

TCCCGAGACTTTGGAAGTTCTCAGCTATTACTTTATTACATAGGATTTCTGTGTCTTTTCTCATCTCT
TTTCCTTTTGGAAAGACCCCCAAAGGGAAAGTGTCCCCTGCGCTGCTCCTGCTCTAAAGACAGCGCCC
TGTGTGAGGGCTCCCCGGACCTGCCCGTCAGCTTCTCTCCGACCCTGCTGTCACTGTCACTCGTCAGG
ACGGGAGTCACCCAGCTGAAGGCCGGCAGCTTCCTGAGAATTCCGTCTCTGCACCTGCTGCTCTTCAC
CTCCAACTCCTTCTCCGTGATTGAGGACGATGCATTTGCGGGCCTGTCCCACCTGCAGTACCTGTTCA
TCGAGGACAATGAGATTGGCTCCATCTCTAAGAATGCCCTCAGAGGACTTCGCTCGCTTACACACCTG
AGCCTGGCCAATAACCATCTGGAGACCCTCCCCAGATTCCTGTTCCGAGGCCTGGACACCCTTACTCA
TGTGGACCTCCGCGGGAACCCGTTCCAGTGTGACTGCCGCGTCCTCTGGCTCCTGCAGTGGATGCCCA
CCGTGAATGCCAGCGTGGGGACCGGCGCCTGTGCGGGCCCCGCCTCCCTGAGCCACATGCAGCTCCAC
CACCTCGACCCCAAGACTTTCAAGTGCCCACCCACAGAGCTGTCCTGGTTCCAGACGGTGGGGGAGTC
GGCACTGAGCGTAGAGCCCTTCTCCTACCAAGGGGAGCCTCACATTGTGCTGGCACAGCCCTTCGCCG
GCCGCTGCCTGATTCTCTCCTGGGACTACAGCCTGCAGCGCTTCCGGCCCGAGGAAGAGCTGCCCACA
GCGGCCTCCGTGGTGTCCTGCAAGCCACTGGTGCTGGGCCCGAGCCTCTTCGTGCTGGCTGCCCGCCT
GTGGGGGGGCTCACAGCTGTGGGCCCGGCCCAGTCCCGGCCTGCGCCTGGCCCCAACGCAGACCCTGG
CCCCGCGGCGGCTGCTGCGGCCCAATGACGCCGAGCTCCTGTGGCTGGAAGGGCAACCCTGCTTCGTG
GTGGCCGATGCCTCCAAGGCGGGCAGCACCACGCTGCTGTGCCGCGACGGGCCCGGCTTTTACCCGCA
CCAGAGCCTGCACGCCTGGCACCGGGACACGGACGCTGAGGCCCTGGAGCTGGACGGCCGGCCCCACC
TGCTGCTGGCCTCGGCTTCCCAGCGGCCCGTGCTCTTCCACTGGACCGGTGGCCGCTTCGAGAGACGC
ACGGACATCCCCGAGGCCGAGGATGTCTATGCCACACGTCACTTCCAGGCTGGTGGGGACGTGTTCCT
GTGCCTCACACGCTACATTGGGGACTCCATGGTGATGCGCTGGGACGGCTCCATGTTTCGTCTGCTGC
AGCAACTTCCCTCGCGCGGTGCCCACGTCTTCCAGCCACTGCTCATCGCCAGGGACCAGTTGGCCATC
CTAGGCAGCGACTTCGCCTTCAGCCAGGTCCTCCGCCTTGAGCCTGACAAGGGGCTCCTGGAGCCACT
GCAGGAGCTGGGGCCTCCGGCCCTGGTGGCCCCCCGTGCCTTTGCCCACATCACTATGGCCGGCAGAC
GCTTCCTCTTTGCTGCTTGCTTTAAGGGCCCCACACAGATCTACCAGCATCACGAGATCGACCTCAGT
GCCTGAGACCACCAACGGGACTCTGGGCATGGCTGGGGCCCCTGGACGGCCCCTTGGCTGGCTCCTGG
CCCTACTTGGGGTGATGGTCCGCCTGTGAGCTGCTGACCGTGGGCCACGTTCATCAGCCACACGTCTA
GGCCTTAAGCCCACTTCTTAAAGGATCTGCACCCATGGGGGGACATAGAGGGTCCCAGCCTACTGGAC
CTCCCGAGCTGCCCTTCAGGTCTAAAGCAACATCTGGACATCTCGGCTGGGGCAGATTCCCCATCAAA
GGCAACCTGGGGGGTGCAGGCTGGTGCACTGCATGGAGTCGAGGTGGAGGTCACGTGCAGAGCTCCAT
GAGGTCCCGGAGCCCCCCTCTCAACCCGCTCCTACCCCGCTGGTCCCTCTGAAAGCACCGAGGGGTTG
ATGGCATTCCTTTCCCATTCTAAACTCCACGCAGCCAATCGCTTTCCTGGAGGAGCGCCAACCTCG

| | | | |
|---|---|---|---|
| NOV34g, SNP13374715 of | SEQ ID NO:832 | 545 aa | MW at 60447.7 kD |
| CG56077-06, Protein Sequence | SNP Pos: 421 | | SNP Change: Arg to Arg |

SRDFGSSQLLLYYIGFLCLFSSLFLLERPPKGKCPLRCSCSKDSALCEGSPDLPVSFSPTLLSLSLVR
TGVTQLKAGSFLRIPSLHLLLFTSNSFSVIEDDAFAGLSHLQYLFIEDNEIGSISKNALRGLRSLTHL
SLANNHLETLPRFLFRGLDTLTHVDLRGNPFQCDCRVLWLLQWMPTVNASVGTGACAGPASLSHMQLH
HLDPKTFKCPPTELSWFQTVGESALSVEPFSYQGEPHIVLAQPFAGRCLILSWDYSLQRFRPEEELPT
AASVVSCKPLVLGPSLFVLAARLWGGSQLWARPSPGLRLAPTQTLAPRRLLRPNDAELLWLEGQPCFV
VADASKAGSTTLLCRDGPGFYPHQSLHAWHRDTDAEALELDGRPHLLLASASQRPVLFHWTGGRFERR
TDIPEAEDVYATRHFQAGGDVFLCLTRYIGDSMVMRWDGSMFRLLQQLPSRGABVFQPLLIARDQLAI
LGSDFAFSQVLRLEPDKGLLEPLQELGPPALVAPRAFAHITMAGRRFLFAACFKGPTQIYQ
HHEIDLSA

| | | |
|---|---|---|
| NOV34h, SNP13374714 of | SEQ ID NO:833 | 2106 bp |
| CG56077-06, DNA Sequence | ORF Start: at 1 | ORF Stop: TGA at 1636 |
| | SNP Pos: 1402 | SNP Change: C to T |

TCCCGAGACTTTGGAAGTTCTCAGCTATTACTTTATTACATAGGATTTCTGTGTCTTTTCTCATCTCT
TTTCCTTTTGGAAAGACCCCCAAAGGGAAAGTGTCCCCTGCGCTGCTCCTGCTCTAAAGACAGCGCCC
TGTGTGAGGGCTCCCCGGACCTGCCCGTCAGCTTCTCTCCGACCCTGCTGTCACTGTCACTCGTCAGG
ACGGGAGTCACCCAGCTGAAGGCCGGCAGCTTCCTGAGAATTCCGTCTCTGCACCTGCTGCTCTTCAC
CTCCAACTCCTTCTCCGTGATTGAGGACGATGCATTTGCGGGCCTGTCCCACCTGCAGTACCTGTTCA
TCGAGGACAATGAGATTGGCTCCATCTCTAAGAATGCCCTCAGAGGACTTCGCTCGCTTACACACCTG
AGCCTGGCCAATAACCATCTGGAGACCCTCCCCAGATTCCTGTTCCGAGGCCTGGACACCCTTACTCA
TGTGGACCTCCGCGGGAACCCGTTCCAGTGTGACTGCCGCGTCCTCTGGCTCCTGCAGTGGATGCCCA
CCGTGAATGCCAGCGTGGGGACCGGCGCCTGTGCGGGCCCCGCCTCCCTGAGCCACATGCAGCTCCAC
CACCTCGACCCCAAGACTTTCAAGTGCCCACCCACAGAGCTGTCCTGGTTCCAGACGGTGGGGGAGTC
GGCACTGAGCGTAGAGCCCTTCTCCTACCAAGGGGAGCCTCACATTGTGCTGGCACAGCCCTTCGCCG
GCCGCTGCCTGATTCTCTCCTGGGACTACAGCCTGCAGCGCTTCCGGCCCGAGGAAGAGCTGCCCACA
GCGGCCTCCGTGGTGTCCTGCAAGCCACTGGTGCTGGGCCCGAGCCTCTTCGTGCTGGCTGCCCGCCT
GTGGGGGGGCTCACAGCTGTGGGCCCGGCCCAGTCCCGGCCTGCGCCTGGCCCCAACGCAGACCCTGG
CCCCGCGGCGGCTGCTGCGGCCCAATGACGCCGAGCTCCTGTGGCTGGAAGGGCAACCCTGCTTCGTG
GTGGCCGATGCCTCCAAGGCGGGCAGCACCACGCTGCTGTGCCGCGACGGGCCCGGCTTTTACCCGCA
CCAGAGCCTGCACGCCTGGCACCGGGACACGGACGCTGAGGCCCTGGAGCTGGACGGCCGGCCCCACC
TGCTGCTGGCCTCGGCTTCCCAGCGGCCCGTGCTCTTCCACTGGACCGGTGGCCGCTTCGAGAGACGC
ACGGACATCCCCGAGGCCGAGGATGTCTATGCCACACGCCACTTCCAGGCTGGTGGGGACGTGTTCCT
GTGCCTCACACGCTACATTGGGGACTCCATGGTGATGCGCTGGGACGGCTCCATGTTTCGTCTGCTGC
AGCAACTTCCCTCGCGCGGTGCCCACGTCTTCCAGCCACTGTTCATCGCCAGGGACCAGTTGGCCATC
CTAGGCAGCGACTTCGCCTTCAGCCAGTCCTCCGCCTTGAGCCTGACAAGGGGCTCCTGGAGGCCACT
GCAGGAGCTGGGGCCTCCGGCCCTGGTGGCCCCCCGTGCCTTTGCCCACATCACTATGGCCGGCAGAC
GCTTCCTCTTTGCTGCTTGCTTTAAGGGCCCCACACAGATCTACCAGCATCACGAGATCGACCTCAGT
GCCTGAGACCACCACCGGGACTCTGGGCATGGCTGGGGCCCCTGGACGGCCCCTTGGCTGGCTCCTGG
CCCTACTTGGGGTGATGGTCCGCCTGTGAGCTGCTGACCGTGGGCCACGTTCATCAGCCACACGTCTA
GGCCTTAAGCCCACTTCTTAAAGGATCTGCACCCATGGGGGGACATAGAGGGTCCCAGCCTACTGGAC
CTCCCGAGCTGCCCTTCAGGTCTAAAGCAACATCTGGACATCTCGGCTGGGGCAGATTCCCCATCAAA
GGCAACCTGGGGGGTGCAGGCTGGTGCACTGCATGGAGTCGAGGTGGAGGTCACGTGCAGAGCTCCAT
GAGGTCCCGGAGCCCCCCTCTCAACCCGCTCCTACCCCGCTGGTCCCTCTGAAAGCACCGAGGGGTTG
ATGGCATTCCTTTCCCATTCTAAACTCCACGCAGCCAATCGCTTTCCTGGAGGAGCGCCAACCTCG

| | | | |
|---|---|---|---|
| NOV34h, SNP13374714 of | SEQ ID NO:834 | 545 aa | MW at 60481.7 kD |
| CG56077-06, Protein Sequence | SNP Pos: 468 | | SNP Change: Leu to Phe |

SRDFGSSQLLLYYIGFLCLFSSLFLLERPPKGKCPLRCSCSKDSALCEGSPDLPVSFSPTLLSLSLVR
TGVTQLKAGSFLRIPSLHLLLFTSNSFSVIEDDAFAGLSHLQYLFIEDNEIGSISKNALRGLRSLTHL
SLANNHLETLPRFLFRGLDTLTHVDLRGNPFQCDCRVLWLLQWMPTVNASVGTGACAGPASLSHMQLH
HLDPKTFKCPPTELSWFQTVGESALSVEPFSYQGEPHIVLAQPFAGRCLILSWDYSLQRFRPEEELPT
AASVVSCKPLVLGPSLFVLAARLWGGSQLWARPSPGLRLAPTQTLAPRRLLRPNDAELLWLEGQPCFV
VADASKAGSTTLLCRDGPGFYPHQSLHAWHRDTDAEALELDGRPHLLLASASQRPVLFHWTGGRFERR
TDIPEAEDVYATRHFQAGGDVFLCLTRYIGDSMVMRWDGSMFRLLQQLPSRGAHVFQPLFIARDQLAI
LGSDFAFSQVLRLEPDKGLLEPLQELGPPALVAPRAFAHITMAGRRFLFAACFKGPTQIYQ
HHEIDLSA

A ClustalW comparison of the above protein sequences yields the following sequence alignment shown in Table 34B.

TABLE 34B
Comparison of the NOV34 protein sequences.

| | | |
|---|---|---|
| NOV34a | ------------------------------SRDFGSSQLLLYYIGFLCLFSSLFLLERPP | (SEQ ID NO:820) |
| NOV34b | MDSPAALRIQGQAGWRSGAVARNLCLYSVLVITEG---------------------RQPP | (SEQ ID NO:822) |
| NOV34c | ----------------------------SRDFGSSQLLLYYIGFLCLFSSLFLLEIGRPP | (SEQ ID NO:824) |
| NOV34d | --------------------------------TEG---------------------RQPP | (SEQ ID NO:826) |
| NOV34e | --------------------------------TEG---------------------RQPP | (SEQ ID NO:828) |
| NOV34f | --------------------------------TEG---------------------RQPP | (SEQ ID NO:830) |
| NOV34a | KGKCPLRCSCSKDSALCEGSPDLPVSFSPTLLSL------SLVRTGVTQLKAGSFLRIPS | |
| NOV34b | KGKCPLRCSCSKDSALCEGSPDLPVSFSPTLLSL------SLVRTGVTQLKAGSFLRIPS | |
| NOV34c | KGKCPLRCSCSKDSALCEGSPDLPVSFSPTLLSLTAHIPSSLVRTGVTQLKAGSFLRIPS | |
| NOV34d | KGKCPLRCSCSKDSALCEGSPDLPVSFSPTLLSL------SLVRTGVTQLKAGSFLRIPS | |
| NOV34e | KGKCPLRCSCSKDSALCEGSPDLPVSFSPTLLSL------SLVRTGVTQLKAGSFLRIPS | |
| NOV34f | KGKCPLRCSCSKDSALCEGSPDLPVSFSPTLLSL------SLVRTGVTQLKAGSFLRIPS | |
| NOV34a | LHLLLFTSNSFSVIEDDAFAGLSHLQYLFIEDNEIGSISKNALRGLRSLTHLSLANNHLE | |
| NOV34b | LHLLLFTSNSFSVIEDDAFAGLSHLQYLFIEDNEIGSISKNALRGLRSLTHLSLANNHLE | |
| NOV34c | LHLLLFTSNSFSVIEDDAFAGLSHLQYLFIEDNEIGSISKNALRGLRSLTHLSLANNHLE | |
| NOV34d | LHLLLFTSNSFSVIEDDAFAGLSHLQYLFIEDNEIGSISKNALRGLRSLTHLSLANNHLE | |
| NOV34e | LHLLLFTSNSFSVIEDDAFAGLSHLQYLFIEDNEIGSISKNALRGLRSLTHLSLANNHLE | |
| NOV34f | LHLLLFTSNSFSVIEDDAFAGLSHLQYLFIEDNEIGSISKNALRGLRSLTHLSLANNHLE | |
| NOV34a | TLPRFLFRGLDTLTHVDLRGNPFQCDCRVLWLLQWMPTVNASVGTGACAGPASLSHMQLH | |
| NOV34b | TLPRFLFRGLDTLTHVDLRGNPFQCDCRVLWLLQWMPTVNASVGTGACAGPASLSHMQLH | |
| NOV34c | TLPRFLFRGLDTLTHVDLRGNPFQCDCRVLWLLQWMPTVNASVGTGACAGPASLSHMQLH | |
| NOV34d | TLPRFLFRGLDTLTHVDLRGNPFQCDCRVLWLLQWMPTVNASVGTGACAGPASLSHMQLH | |
| NOV34e | TLPRFLFRGLDTLTHVDLRGNPFQCDCRVLWLLQWMPTVNASVGTGACAGPASLSHMQLH | |
| NOV34f | TLPRFLFRGLDTLTHVDLRGNPFQCDCRVLWLLQWMPTVNASVGTGACAGPASLSHMQLH | |
| NOV34a | HLDPKTFKCPP-----------------------------------TELSWFQTVGESAL | |
| NOV34b | HLDPKTFKCRA-----------------------------------IELSWFQTVGESAL | |
| NOV34c | HLDPKTFKCTAASVVSCKPLVLGPSLFVLAARLWGGSQLWARPSPGLRLAPTQTLAPRRL | |
| NOV34d | HLDPKTFKCRA-----------------------------------IELSWFQTVGESAL | |
| NOV34e | HLDPKTFKCRA-----------------------------------IELSWFQTVGESAL | |
| NOV34f | HLDPKTFKCRA-----------------------------------IELSWFQTVGESAL | |
| NOV34a | S-------------------------------------------------VEPFSYQGEP | |
| NOV34b | S-------------------------------------------------VEPFSYQGEP | |
| NOV34c | LRPNDAELLWLEGQPCFVVADASKAGSTTCSASGPRKSCPSLHAWHRDTDAEALELDGRP | |
| NOV34d | S-------------------------------------------------VEPFSYQGEP | |
| NOV34e | S-------------------------------------------------VEPFSYQGEP | |
| NOV34f | S-------------------------------------------------VEPFSYQGEP | |
| NOV34a | HIVLAQP--------------------------------------------FAGRCLILS | |
| NOV34b | HIVLAQP--------------------------------------------FAGRCLILS | |
| NOV34c | HLLLASASQRPVLFHWTGGRFERRTDIPEAEDVYATRHFQAGGDVFLCLTRYIGDSMVMR | |
| NOV34d | HIVLAQP--------------------------------------------FAGRCLILS | |
| NOV34e | HIVLAQP--------------------------------------------FAGRCLILS | |
| NOV34f | HIVLAQP--------------------------------------------FAGRCLILS | |
| NOV34a | WDYSLQRFR--------------------------------------------PEEELPT | |
| NOV34b | WDYSLQRFR--------------------------------------------PEEELPA | |
| NOV34c | WDGSMFRLLQQLPSRGAHVFQPLLIARDQLAILGSDFAFSQVLRLEPDKGLLEPLQELGP | |
| NOV34d | WDYSLQRFR--------------------------------------------PEEEL-- | |
| NOV34e | WDYSLQRFR--------------------------------------------PEEEL-- | |
| NOV34f | WDYSLQRFR--------------------------------------------PEEEL-- | |
| NOV34a | AASVVSCKPLVLGPSLFVLAARLWGGSQLWARPSPGLRLAPTQTLAPRRLLRPNDAELLW | |
| NOV34b | ASVVSCKPLVLGPSLFVLAARLWGGSQLWARPSPGLRLAPTQTLAPRRLLRPNDAELLWL | |
| NOV34c | LALVAPRAFAHITMAGRRFLFAACFKGPTQIYQHHEIDLSA------------------- | |
| NOV34d | ------------------------------------------------------------ | |
| NOV34e | ------------------------------------------------------------ | |
| NOV34f | ------------------------------------------------------------ | |
| NOV34a | LEGQPCFVVADASKAGSTTLLCRDGPGFYPHQSLHAWHRDTDAEALELDGRPHLLLASAS | |
| NOV34b | EGQPCFVVADASKAGSTTLLCRDGPGFYPHQSLHAWHRDTDAEALELDGRPHLLLASASQ | |
| NOV34c | ------------------------------------------------------------ | |
| NOV34d | ------------------------------------------------------------ | |
| NOV34e | ------------------------------------------------------------ | |
| NOV34f | ------------------------------------------------------------ | |
| NOV34a | QRPVLFHWTGGRFERRTDIPEAEDVYATRHFQAGGDVFLCLTRYIGDSMVMRWDGSMFRL | |
| NOV34b | RPVLFHWTGGRFERRTDIPRAEDVYATRHFQAGGDVFLCLTRYIGDSMVMRWDGSMFRLL | |
| NOV34c | ------------------------------------------------------------ | |
| NOV34d | ------------------------------------------------------------ | |
| NOV34e | ------------------------------------------------------------ | |
| NOV34f | ------------------------------------------------------------ | |
| NOV34a | LQQLPSRGAHVFQPLLIARDQLAILGSDFAFSQVLRLEPDKGLLEPLQELGPPALVAPRA | |
| NOV34b | QQLPSRGAHVFQPLLIARDQLAILGSDFAFSQVLRLEPDKGLLEPLQELGPPALVAPRAF | |
| NOV34c | ------------------------------------------------------------ | |
| NOV34d | ------------------------------------------------------------ | |
| NOV34e | ------------------------------------------------------------ | |
| NOV34f | ------------------------------------------------------------ | |
| NOV34a | FAHITMAGRPFLFAACFKGPTQIYQHHEIDLSA | |
| NOV34b | AHITMAGRRFLFAACFKGPTQIYQHHEIDLSA- | |
| NOV34c | --------------------------------- | |
| NOV34d | --------------------------------- | |
| NOV34e | --------------------------------- | |
| NOV34f | --------------------------------- | |

Further analysis of the NOV34a protein yielded the following properties shown in Table 34C.

TABLE 34C
Protein Sequence Properties NOV34a

| | |
|---|---|
| SignalP analysis: | Cleavage site between residues 26 and 27 |
| PSORT II analysis: | |

| PSG: | a new signal peptide prediction method | |
|---|---|---|
| | N-region: | length 3; pos. chg 1; neg. chg 1 |
| | H-region: | length 23; peak value 10.35 |
| | PSG score: | 5.95 |

| | |
|---|---|
| GvH: | von Heijne's method for signal seq. recognition |
| | GvH score (threshold: −2.1): −5.07 |
| | possible cleavage site: between 25 and 26 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 2 | | |
| | Number of TMS(s) for threshold 0.5: 1 | | |
| | INTEGRAL | Likelihood = | Transmembrane 9-25 |
| | | −5.57 | |

| | |
|---|---|
| PERIPHERAL | Likelihood = 2.70 (at 82) |

| | |
|---|---|
| | ALOM score: −5.57 (number of TMSs: 1) |
| MTOP: | Prediction of membrane topology (Hartmann et al.) |
| | Center position for calculation: 16 |
| | Charge difference: 2.0 C(3.0)-N(1.0) |
| | C > N: C-terminal side will be inside |

>>>Caution: Inconsistent mtop result with signal peptide
>>> membrane topology: type 1b (cytoplasmic tail 9 to 545)

| MITDISC: | discrimination of mitochondrial targeting seq | | | |
|---|---|---|---|---|
| | R content: | 1 | Hyd Moment(75): | 11.37 |
| | Hyd Moment(95): | 7.33 | G content: | 2 |
| | D/E content: | 2 | S/T content: | 5 |

| | |
|---|---|
| | Score: −4.71 |
| Gavel: | prediction of cleavage sites for mitochondrial preseq |
| | R-2 motif at 47 LRC\|SC |

NUCDISC: discrimination of nuclear localization signals

| | |
|---|---|
| | pat4: none |
| | pat7: none |
| | bipartite: none |
| | content of basic residues: 8.8% |
| | NLS Score: −0.47 |
| KDEL: | ER retention motif in the C-terminus: none |

ER Membrane Retention Signals:

XXRR-like motif in the N-terminus: RDFG

none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: found

LL at 9
LL at 10
LL at 25
LL at 61
LL at 87
LL at 88
LL at 176
LL at 322
LL at 330
LL at 352
LL at 386
LL at 387

checking 63 PROSITE DNA binding motifs:

Leucine zipper pattern (PS00029): *** found ***
LSLANNHLETLPRFLFRGLDTL at 136

none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 89 |
| COIL: | Lupas's algorithm to detect coiled-coil regions |
| | total: 0 residues |

Final Results (k = 9/23):

```
34.8%: nuclear
21.7%: mitochondrial
21.7%: cytoplasmic
  8.7%: vesicles of secretory system
  4.3%: vacuolar
  4.3%: endoplasmic reticulum
  4.3%: peroxisomal
```

>> prediction for CG56077-06 is nuc (k = 23)

A search of the NOV34a protein against the Geneseq database, a proprietary database that contains sequences published in patents and patent publication, yielded several homologous proteins shown in Table 34D.

TABLE 34D
Geneseq Results for NOV34a

| | | NOV34a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB09515 | Leu-rich glioma-inactivated protein-like | 17 . . . 545 | 518/531 (97%) | 0.0 |
| | NOV7a protein, SEQ ID NO: 28 - Homo | 24 . . . 553 | 521/531 (97%) | |
| | sapiens, 553 aa. [WO200253742-A2, | | | |
| | 11-JUL-2002] | | | |
| ABB98246 | Human CGDD SEQ ID NO 4 - Homo | 5 . . . 545 | 519/541 (95%) | 0.0 |
| | sapiens, 537 aa. [WO200248368-A2, | 3 . . . 537 | 523/541 (95%) | |
| | 20-JUN-2002] | | | |
| AAE25347 | Human LP polypeptide, LP188 - Homo | 5 . . . 545 | 519/541 (95%) | 0.0 |
| | sapiens, 537 aa. [WO200248361-A2, | 3 . . . 537 | 523/541 (95%) | |
| | 20-JUN-2002] | | | |
| ABG91403 | Primate LP286 - Mammalia, 513 aa. | 5 . . . 545 | 495/541 (91%) | 0.0 |
| | [WO200263009-A2, 15-AUG-2002] | 3 . . . 513 | 499/541 (91%) | |
| ABU11596 | Human MDDT polypeptide SEQ ID 543 - | 55 . . . 545 | 482/493 (97%) | 0.0 |
| | Homo sapiens, 567 aa. | 76 . . . 567 | 483/493 (97%) | |
| | [WO200279449-A2, 10-OCT-2002] | | | |

In a BLAST search of public sequence databases, the NOV34a protein was found to have homology to the proteins shown in the BLASTP data in Table 34E.

TABLE 34E
Public BLASTP Results for NOV34a

| | | NOV34a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8N135 | LGI4 (Leucine-rich glioma inactivated | 5 . . . 545 | 519/541 (95%) | 0.0 |
| | protein 4) (LGI1-like protein 3) - Homo | 3 . . . 537 | 523/541 (95%) | |
| | sapiens (Human), 537 aa. | | | |
| Q8K1S1 | LGI1-like protein 3 - Mus musculus | 16 . . . 545 | 460/530 (86%) | 0.0 |
| | (Mouse), 537 aa. | 9 . . . 537 | 491/530 (91%) | |
| Q8K4Y8 | Leucine-rich glioma inactivated | 16 . . . 545 | 458/530 (86%) | 0.0 |
| | protein 4 - Mus musculus (Mouse), | 9 . . . 537 | 489/530 (91%) | |
| | 537 aa. | | | |
| Q9JIA1 | Leucine-rich glioma-inactivated 1 | 1 . . . 545 | 275/556 (49%) | e−152 |
| | protein - Mus musculus (Mouse), 557 aa. | 6 . . . 557 | 375/556 (66%) | |
| Q8K4Y5 | Leucine-rich glioma inactivated | 16 . . . 545 | 270/540 (50%) | e−151 |
| | protein 1 - Rattus norvegicus (Rat), | 22 . . . 557 | 367/540 (67%) | |
| | 557 aa. | | | |

PFam analysis predicts that the NOV34a protein contains the domains shown in the Table 34F.

TABLE 34F
Domain Analysis of NOV34a

| | | | |
|---|---|---|---|
| | | Identities/ | |
| | | Similarities for | |
| Pfam | NOV34a | the Matched | Expect |
| Domain | Match Region | Region | Value |

| | | | | |
|---|---|---|---|---|
| LRR | 108 . . . 131 | 7/25 | (28%) | 0.099 |
| | | 21/25 | (84%) | |
| LRR | 132 . . . 155 | 10/25 | (40%) | 0.058 |
| | | 20/25 | (80%) | |
| LRRCT | 165 . . . 214 | 21/54 | (39%) | 1.1e−10 |
| | | 36/54 | (67%) | |
| EPTP | 222 . . . 354 | 30/147 | (20%) | 1.7e−07 |
| | | 73/147 | (50%) | |
| EPTP | 405 . . . 537 | 58/144 | (40%) | 8e−39 |
| | | 90/144 | (62%) | |

### Example 35

The NOV35 clone was analyzed, and the nucleotide and encoded polypeptide sequences are shown in Table 35A.

TABLE 35A
NOV35 Sequence Analysis

| | | |
|---|---|---|
| NOV35a, CG56110-03 | SEQ ID NO:835 | 874 bp |
| DNA Sequence | ORF Start: ATG at 1 | ORF Stop: TAA at 871 |

ATGAGGATATTTGCTGTCTTTATATTCATGACCTACTGGCATTTGCTGAACGCATTTACTGTCACGGT
TCCCAAGGACCTATATGTGGTAGAGTATGGTAGCAATATGACAATTGAATGCAAATTCCCAGTAGAAA
AACAATTAGACCTGGCTGCACTAATTGTCTATTGGGAAATGGAGGATAAGAACATTATTCAATTTGTG
CATGGAGAGGAAGACCTGAAGGTTCAGCATAGTAGCTACAGACAGAGGGCCCGGCTGTTGAAGGACCA
GCTCTCCCTGGGAAATGCTGCACTTCAGATCACAGATGTGAAATTGCAGGATGCAGGGGTGTACCGCT
GCATGATCAGCTATGGTGGTGCCGACTACAAGCGAATTACTGTGAAAGTCAATGCCCCATACAACAAA
ATCAACCAAAGAATTTTGGTTGTGGATCCAGTCACCTCTGAACATGAACTGACATGTCAGGCTGAGGG
CTACCCCAAGGCCGAAGTCATCTGGACAAGCAGTGACCATCAAGTCCTGAGTGGTAAGACCACCACCA
CCAATTCCAAGAGAGAGGAGAAGCTTTTCAATGTGACCAGCACACTGAGAATCAACACAACAACTAAT
GAGATTTTCTACTGCACTTTTAGGAGATTAGATCCTGAGGAAAACCATACAGCTGAATTGGTCATCCC
AGAACTACCTCTGGCACATCCTCCAAATGAAAGGACTCACTTGGTAATTCTGGGAGCCATCTTATTAT
GCCTTGGTGTAGCACTGACATTCATCTTCCGTTTAAGAAAAGGGAGAATGATGGATGTGAAAAAATGT
GGCATCCAAGATACAAACTCAAAGAAGCAAAGTGATACACATTTGGAGGAGACGTAAA

| | | | |
|---|---|---|---|
| NOV35a, CG56110-03 | SEQ ID NO:835 | 290 aa | MW at 33275.0 kD |
| Protein Sequence | | | |

MRIFAVFIFMTYWHLLNAFTVTVPKDLYVVEYGSNMTIECKFPVEKQLDLAALIVYWEMEDKNIIQFV
HGEEDLKVQHSSYRQHARLLKDQLSLGNAALQITDVKLQDAGVYRCMISYGGADYKRITVKVNAPYNK
INQRILVVDPVTSEHELTCQAEGYPKAEVIWTSSDHQVLSGKTTTTNSKREEKLFNVTSTLRINTTTN
EIFYCTFRRLDPEENHTAELVIPELPLAHPPNERTHLVILGAILLCLGVALTFIFRLRKGRMMDVKKC
GIQDTNSKKQSDTHLEET

| | | |
|---|---|---|
| NOV35b, CG56110-07 | SEQ ID NO:837 | 720 bp |
| DNA Sequence | ORF Start: ATG at 3 | ORF Stop: TAG at 510 |

TCATGACCTACTGGCATTTGCTGAACGCATTTACTGTCACGGTTCCCAAGGACCTATATGTGGTAGAG
TATGGTAGCAATATGACAATTGAATGCAAATTCCCAGTAGAAAAACAATTAGACCTGGCTGCACTAAT
TGTCTATTGGGAAATGGAGGATAAGAACATTATTCAATTTGTGCATGGAGAGGAAGACCTGAAGGTTC
AGCATAGTAGCTACAGACAGAGGGCCCGGCTGTTGAAGGACCAGCTCTCCCTGGGAAATGCTGCACTT
CAGATCACAGATGTGAAATTGCAGGATGCAGGGGTGTACCGCTGCATGATCAGCTATGGTGGTGCCGA
CTACAAGCGAATTACTGTGAAAGTCAATGCCCCATACAACAAAATCAACCAAAGAATTTTGGTTGTGG
ATCCAGTCACCTCTGAACATGAACTGACATGTCAGGCTGAGGGCTACCCCAAGGCCGAAGTCATCTGG
ACAAGCAGTGACCATCAAGTCCTGAGTGGAGATTAGATCCTGAGGAAAACCATACAGCTGAATTGGTC
ATCCCAGAACTACCTCTGGCACATCCTCCAAATGAAAGGACTCACTTGGTAATTCTGGGAGCCATCTT
ATTATGCCTTGGTGTAGCACTGACATTCATCTTCCGTTTAAGAAAAGGGAGAATGATGGATGTGAAAA
AATGTGGCATCCAAGATACAAACTCAAAGAAGCAAAGTGG

| | | | |
|---|---|---|---|
| NOV35b, CG56110-07 | SEQ ID NO:838 | 169 aa | MW at 19327.9 kD |
| Protein Sequence | | | |

MTYWHLLNAFTVTVPKDLYVVEYGSNMTIECKFPVEKQLDLAALIVYWEMEDKNIIQFVHGEEDLKVQ
HSSYRQRARLLKDQLSLGNAALQITDVKLQDAGVYRCMISYGGADYKRITVKVNAPYNKINQRILVVD
PVTSEHELTCQAEGYPKARVIWTSSDHQVLSGD

| | | |
|---|---|---|
| NOV35c, 274082305 | SEQ ID NO:839 | 900 bp |
| DNA Sequence | ORF Start: at 2 | ORF Stop: TAA at 887 |

CACCAGATCTCCCACCATGAGGATATTTGCTGTCTTTATATTCATGACCTACTGGCATTTGCTGAACG
CATTTACTGTCACGGTTCCCAAGGACCTATATGTGGTAGAGTATGGTAGCAATATGACAATTGAATGC
AAATTCCCAGTAGAAAAACAATTAGACCTGGCTGCACTAATTGTCTATTGGGAAATGGAGGATAAGAA
CATTATTCAATTTGTGCATGGAGAGGAAGACCTGAAGGTTCAGCATAGTAGCTACAGACAGAGGGCCC
GGCTGTTGAAGGACCAGCTCTCCCTGGGAAATGCTGCACTTCAGATCACAGATGTGAAATTGCAGGAT
GCAGGGGTGTACCGCTGCATGATCAGCTATGGTGGTGCCGACTACAAGCGAATTACTGTGAAAGTCAA
TGCCCCATACAACAAAATCAACCAAAGAATTTTGGTTGTGGATCCAGTCACCTCTGAACATGAACTGA
CATGTCAGGCTGAGGGCTACCCCAAGGCCGAAGTCATCTGGACAAGCAGTGACCATCAAGTCCTGAGT
GGTAAGACCACCACCACCAATTCCAAGAGAGAGGAGAAGCTTTTCAATGTGACCAGCACACTGAGAAT
CAACACAACAACTAATGAGATTTTCTACTGCACTTTTAGGAGATTAGATCCTGAGGAAAACCATACAG
CTGAATTGGTCATCCCAGAACTACCTCTGGCACATCCTCCAAATGAAAGGACTCACTTGGTAATTCTG
GGAGCCATCTTATTATGCCTTGGTGTAGCACTGACATTCATCTTCCGTTTAAGAAAAGGGAGAATGAT
GGATGTGAAAAAATGTGGCATCCAAGATACAAACTCAAAGAAGCAAAGTGATACACATTTGGAGGAGA
CGTAAAGTCGACGGCC

| | | | |
|---|---|---|---|
| NOV35c, 274082305 | SEQ ID NO:840 | 295 aa | MW at 33817.6 kD |
| Protein Sequence | | | |

TRSPTMRIFAVFIFMTYWHLLNAFTVTVPKDLYVVEYGSNMTIECKFPVEKQLDLAALIVYWEMEDKN
IIQFVHGEEDLKVQHSSYRQRARLLKDQLSLGNAALQITDVKLQDAGVYRCMISYGGADYKRITVKVN
APYNKINQRILVVDPVTSEHELTCQAEGYPKAEVIWTSSDHQVLSGKTTTTNSKREEKLFNVTSTLRI
NTTTNEIFYCTFRRLDPEENHTAELVIPELPLAHPPNERTHLVILGAILLCLGVALTFIFRLRKGRMM
DVKKCGIQDTNSKKQSDTHLEET

| | | |
|---|---|---|
| NOV35d, CG56110-01 | SEQ ID NO:841 | 873 bp |
| DNA Sequence | ORF Start: ATG at 1 | ORF Stop: TAA at 871 |

ATGAGGATATTTGCTGTCTTTATATTCATGACCTACTGGCATTTGCTGAACGCATTTACTGTCACGGT
TCCCAAGGACCTATATGTGGTAGAGTATGGTAGCAATATGACAATTGAATGCAAATTCCCAGTAGAAA
AACAATTAGACCTGGCTGCACTAATTGTCTATTGGGAAATGGAGGATAAGAACATTATTCAATTTGTG
CATGGAGAGGAAGACCTGAAGGTTCAGCATAGTAGCTACAGACAGAGGGCCCGGCTGTTGAAGGACCA
GCTCTCCCTGGGAAATGCTGCACTTCAGATCACAGATGTGAAATTGCAGGATGCAGGGGTGTACCGCT
GCATGATCAGCTATGGTGGTGCCGACTACAAGCGAATTACTGTGAAAGTCAATGCCCCATACAACAAA
ATCAACCAAAGAATTTTGGTTGTGGATCCAGTCACCTCTGAACATGAACTGACATGTCAGGCTGAGGG
CTACCCCAAGGCCGAAGTCATCTGGACAAGCAGTGACCATCAAGTCCTGAGTGGTAAGACCACCACCA
CCAATTCCAAGAGAGAGGAGAAGCTTTTCAATGTGACCAGCACACTGAGAATCAACACAACAACTAAT
GAGATTTTCTACTGCACTTTTAGGAGATTAGATCCTGAGGAAAACCATACAGCTGAATTGGTCATCCC
AGAACTACCTCTGGCACATCCTCCAAATGAAAGGACTCACTTGGTAATTCTGGGAGCCATCTTATTAT
GCCTTGGTGTAGCACTGACATTCATCTTCCGTTTAAGAAAAGGGAGAATGATGGATGTGAAAAAATGT
GGCATCCAAGATACAAACTCAAAGAAGCAAAGTGATACACATTTGGAGGAGACGTAA

| | | | |
|---|---|---|---|
| NOV35d, CG56110-01 | SEQ ID NO:842 | 290 aa | MW at 33275.0 kD |
| Protein Sequence | | | |

MRIFAVFIFMTYWHLLNAFTVTVPKDLYVVEYGSNMTIECKFPVEKQLDLAALIVYWEMEDKNIIQFV
HGEEDLKVQHSSYRQRAELLKDQLSLGNAALQITDVKLQDAGVYRCMISYGGADYKRITVKVNAPYNK
INQRILVVDPVTSEHELTCQAEGYPKAEVIWTSSDHQVLSGKTTTTNSKREEKLFNVTSTLRINTTTN
EIFYCTFRRLDPEENHTAELVIPELPLAHPPNERTHLVILGAILLCLGVALTFIFRLRKGRNMDVKKC
GIQDTNSKKQSDTHLEET

| | | |
|---|---|---|
| NOV35e, CG56110-02 | SEQ ID NO:843 | 666 bp |
| DNA Sequence | ORF Start: at 1 | ORF Stop: end of sequence |

TTTACTGTCACGGTTCCCAAGGACCTATATGTGGTAGAGTATGGTAGCAATATGACAATTGAATGCAA
ATTCCCAGTAGAAAAACAATTAGACCTGGCTGCACTAATTGTCTATTGGGAAATGGAGGATAAGAACA
TTATTCAATTTGTGCATGGAGAGGAAGACCTGAAGGTTCAGCATAGTAGCTACAGACAGAGGGCCCGG
CTGTTGAAGGACCAGCTCTCCCTGGGAAATGCTGCACTTCAGATCACAGATGTGAAATTGCAGGATGC
AGGGGTGTACCGCTGCATGATCAGCTATGGTGGTGCCGACTACAAGCGAATTACTGTGAAAGTCAATG
CCCCATACAACAAAATCAACCAAAGAATTTTGGTTGTGGATCCAGTCACCTCTGAACATGAACTGACA
TGTCAGGCTGAGGGCTACCCCAAGGCCGAAGTCATCTGGACAAGCAGTGACCATCAAGTCCTGAGTGG
TAAGACCACCACCACCAATTCCAAGAGAGAGGAGAAGCTTTTCAATGTGACCAGCACACTGAGAATCA
ACACAACAACTAATGAGATTTTCTACTGCACTTTTAGGAGATTAGATCCTGAGGAAAACCATACAGCT
GAATTGGTCATCCCAGAACTACCTCTGGCACATCCTCCAAATGAAAGGACTCAC

| | | | |
|---|---|---|---|
| NOV35e, CG56110-02 | SEQ ID NO:844 | 222 aa | MW at 25431.6 kD |
| Protein Sequence | | | |

FTVTVPKDLYVVEYGSNMTIECKFPVEKQLDLAALIVYWEMEDKNIIQFVHGEEDLKVQHSSYRQRAR
LLKDQLSLGNAALQITDVKLQDAGVYRCMISYGGADYKRITVKVNAPYNKINQRILVVDPVTSEHELT
CQAEGYPKAEVIWTSSDHQVLSGKTTTTNSKREEKLFNVTSTLRINTTTNEIFYCTFRRLDPEENHTA
ELVIPELPLAHPPNERTH

| | | |
|---|---|---|
| NOV35f, CG56110-04 | SEQ ID NO:845 | 745 bp |
| DNA Sequence | ORF Start: ATG at 1 | ORF Stop: TAG at 535 |

ATGAGGATATTTGCTGTCTTTATATTCATGACCTACTGGCATTTGCTGAACGCATTTACTGTCACGGT
TCCCAAGGACCTATATGTGGTAGAGTATGGTAGCAATATGACAATTGAATGCAAATTCCCAGTAGAAA
AACAATTAGACCTGGCTGCACTAATTGTCTATTGGGAAATGGAGGATAAGAACATTATTCAATTTGTG
CATGGAGAGGAAGACCTGAAGGTTCAGCATAGTAGCTACAGACAGAGGGCCCGGCTGTTGAAGGACCA
GCTCTCCCTGGGAAATGCTGCACTTCAGATCACAGATGTGAAATTGCAGGATGCAGGGGTGTACCGCT
GCATGATCAGCTATGGTGGTGCCGACTACAAGCGAATTACTGTGAAAGTCAATGCCCCATACAACAAA
ATCAACCAAAGAATTTTGGTTGTGGATCCAGTCACCTCTGAACATGAACTGACATGTCAGGCTGAGGG
CTACCCCAAGGCCGAAGTCATCTGGACAAGCAGTGACCATCAAGTCCTGAGTGGAGATTAGATCCTGA
GGAAAACCATACAGCTGAATTGGTCATCCCAGAACTACCTCTGGCACATCCTCCAAATGAAAGGACTC
ACTTGGTAATTCTGGGAGCCATCTTATTATGCCTTGGTGTAGCACTGACATTCATCTTCCGTTTAAGA
AAAGGGAGAATGATGGATGTGAAAAAATGTGGCATCCAAGATACAAACTCAAAGAAGCAAAGTGG

| | | | |
|---|---|---|---|
| NOV35f, CG56110-04 | SEQ ID NO:846 | 178 aa | MW at 20453.3 kD |
| Protein Sequence | | | |

MRIFAVFIFMTYWHLLNAFTVTVPKDLYVVEYGSNMTIECKFPVEKQLDLAALIVYWEMEDKNIIQFV
HGEEDLKVQHSSYRQRARLLKDQLSLGNAALQITDVKLQDAGVYRCMISYGGADYKRITVKVNAPYNK
INQRILVVDPVTSEHELTCQAEGYPKAEVIWTSSDHQVLSGD

| | | |
|---|---|---|
| NOV35g, CG56110-05 | SEQ ID NO:847 | 7184 bp |
| DNA Sequence | ORF Start: ATG at 62 | ORF Stop: TAA at 932 |

GCAGCTTCCCGAGGCTCCGCACCAGCCGCGCTTCTGTCCGCCTGCAGGGCATTCCAGAAAGATGAGGA
TATTTGCTGTCTTTATATTCATGACCTACTGGCATTTGCTGAACGCATTTACTGTCACGGTTCCCAAG
GACCTATATGTGGTAGAGTATGGTAGCAATATGACAATTGAATGCAAATTCCCAGTAGAAAAACAATT
AGACCTGGCTGCACTAATTGTCTATTGGGAAATGGAGGATAAGAACATTATTCAATTTGTGCATGGAG
AGGAAGACCTGAAGGTTCAGCATAGTAGCTACAGACAGAGGGCCCGGCTGTTGAAGGACCAGCTCTCC
CTGGGAAATGCTGCACTTCAGATCACAGATGTGAAATTGCAGGATGCAGGGGTGTACCGCTGCATGAT
CAGCTATGGTGGTGCCGACTACAAGCGAATTACTGTGAAAGTCAATGCCCCATACAACAAAATCAACC
AAAGAATTTTGGTTGTGGATCCAGTCACCTCTGAACATGAACTGACATGTCAGGCTGAGGGCTACCCC
AAGGCCGAAGTCATCTGGACAAGCAGTGACCATCAAGTCCTGAGTGGTAAGACCACCACCACCAATTC
CAAGAGAGAGGAGAAGCTTTTCAATGTGACCAGCACACTGAGAATCAACACAACAACTAATGAGATTT
TCTACTGCACTTTTAGGAGATTAGATCCTGAGGAAAACCATACAGCTGAATTGGTCATCCCAGAACTA
CCTCTGGCACATCCTCCAAATGAAAGGACTCACTTGGTAATTCTGGGAGCCATCTTATTATGCCTTGG
TGTAGCACTGACATTCATCTTCCGTTTAAGAAAAGGGAGAATGATGGATGTGAAAAAATGTGGCATCC
AAGATACAAACTCAAAGAAGCAAAGTGATACACATTTGGAGGAGACGTAATCCAGCATTGGAACTTCT
GATCTTCAAGCAGGGATTCTCAACCTGTGGTTTAGGGGTTCATCGGGGCTGAGCGTGACAAGAGGAAG
GAATGGGCCCGTGGGATGCAGGCAATGTGGGACTTAAAAGGCCCAAGCACTGAAAATGGAACCTGGCG
AAAGCAGAGGAGGAGAATGAAGAAAGATGGAGTCAAACAGGGAGCCTGGAGGGAGACCTTGATACTTT
CAAATGCCTGAGGGGCTCATCGACGCCTGTGACAGGGAGAAAGGATACTTCTGAACAAGGAGCCTCCA
AGCAAATCATCCATTGCTCATCCTAGGAAGACGGGTTGAGAATCCCTAATTTGAGGGTCAGTTCCTGC
AGAAGTGCCCTTTGCCTCCACTCAATGCCTCAATTTGTTTTCTGCATGACTGAGAGTCTCAGTGTTGG
AACGGGACAGTATTTATGTATGAGTTTTTCCTATTTATTTTGAGTCTGTGAGGTCTTCTTGTCATGTG
AGTGTGGTTGTGAATGATTTCTTTTGAAGATATATTGTAGTAGATGTTACAATTTTGTCGCCAAACTA
AACTTGCTGCTTAATGATTTGCTCACATCTAGTAAAACATGGAGTATTTGTAAGGTGCTTGGTCTCCT
CTATAACTACAAGTATACATTGGAAGCATAAAGATCAAACCGTTGGTTGCATAGGATGTCACCTTTAT
TTAACCCATTAATACTCTGGTTGACCTAATCTTATTCTCAGACCTCAAGTGTCTGTGCAGTATCTGTT
CCATTTAAATATCAGCTTTACAATTATGTGGTAGCCTACACACATAATCTCATTTCATCGCTGTAACC
ACCCTGTTGTGATAACCACTATTATTTTACCCATCGTACAGCTGAGGAAGCAAACAGATTAAGTAACT
TGCCCAAACCAGTAAATAGCAGACCTCAGACTGCCACCCACTGTCCTTTTATAATACAATTTACAGCT
ATATTTTACTTTAAGCAATTCTTTTATTCAAAAACCATTTATTAAGTGCCCTTGCAATATCAATCGCT
GTGCCAGGCATTGAATCTACAGATGTGAGCAAGACAAAGTACCTGTCCTCAAGGAGCTCATAGTATAA
TGAGGAGATTAACAAGAAAATGTATTATTACAATTTAGTCCAGTGTCATAGCATAAGGATGATGCGAG
GGGAAAACCCGAGCAGTGTTGCCAAGAGGAGGAAATAGGCCAATGTGGTCTGGGACGGTTGGATATAC
TTAAACATCTTAATAATCAGAGTAATTTTCATTTACAAAGAGAGGTCGGTACTTAAAATAACCCTGAA
AAATAACACTGGAATTCCTTTTCTAGCATTATATTTATTCCTGATTTGCCTTTGCCATATAATCTAAT
GCTTGTTTATATAGTGTCTGGTATTGTTTAACAGTTCTGTCTTTTCTATTTAAATGCCACTAAATTTT
AAATTCATACCTTTCCATGATTCAAAATTCAAAAGATCCCATGGGAGATGGTTGGAAAATCTCCACTT
CATCCTCCAAGCCATTCAAGTTTCCTTTCCAGAAGCAACTGCTACTGCCTTTCATTCATATGTTCTTC
TAAAGATAGTCTACATTTGGAAATGTATGTTAAAAGCACGTATTTTTAAAATTTTTTTCCTAAATAGT
AACACATTGTATGTCTGCTGTGTACTTTGCTATTTTTATTTATTTTAGTGTTTCTTATATAGCAGATG
GAATGAATTTGAAGTTCCCAGGGCTGAGGATCCATGCCTTCTTTGTTTCTAAGTTATCTTTCCCATAC
CTTTTCATTATCTTTCATATGATCCAGTATATGTTAAATATGTCCTACATATACATTTAGACAACCAC
CATTTGTTAAGTATTTGCTCTAGGACAGAGTTTGGATTTGTTTATGTTTGCTCAAAAGGAGACCCATG
GGCTCTCCAGGGTGCACTGAGTCAATCTAGTCCTAAAAAGCAATCTTATTATTAACTCTGTATGACAG
AATCATGTCTGGAACTTTTGTTTTCTGCTTTCTGTCAAGTATAAACTTCACTTTGATGCTGTACTTGC
AAAATCACATTTTCTTTCTGGAAATTCCGGCAGTGTACCTTGACTGCTAGCTACCCTGTGCCAGAAAA
GCCTCATTCGTTGTGCTTGAACCCTTGAATGCCACCAGCTGTCATCACTACACAGCCCTCCTAAGAGC
CTTCCTGGAGGTTTCGAGATTCAGATGCCCTGGGAGATCCCAGAGTTTCCTTTCCCTCTTGGCCATAT
TCTGGTGTCAATGACAAGGAGTACCTTGGCTTTGCCACATGTCAAGGCTGAAGAAACAGTGTCTCCAA
CAGAGCTCCTTGTGTTATCTGTTTGTACATGTGCATTTGTACAGTAATTGGTGTGACAGTGTTCTTTG
TGTGAATTACAGGCAAGAATTGTGGCTGAGCAAGGCACATAGTCTACTCAGTCTATTCCTAAGTCCTA
ACTCCTCCTTGTGGTGTTGGATTTGTAAGGCACTTTATCCCTTTTGTCTCATGTTTCATCGTAAATGG
CATAGGCAGAGATGATACCTAATTCTGCATTTGATTGTCACTTTTTGTACCTGCATTAATTTAATAAA
ATATTCTTATTTATTTTGTTACTTGGTAAAAAAAAAAAAAAAGCTTCCCGAGGCTCCGCACCAGCCGC
GCTTCTGTCCGCCTGCAGGGCATTCCAGAAAGATGAGGATATTTGCTGTCTTTATATTCATGACCTAC
TGGCATTTGCTGAACGCATTTACTGTCACGGTTCCCAAGGACCTATATGTGGTAGAGTATGGTAGCAA
TATGACAATTGAATGCAAATTCCCAGTAGAAAAACAATTAGACCTGGCTGCACTAATTGTCTATTGGG
AAATGGAGGATAAGAACATTATTCAATTTGTGCATGGAGAGGAAGACCTGAAGGTTCAGCATAGTAGC
TACAGACAGAGGGCCCGGCTGTTGAAGGACCAGCTCTCCCTGGGAAATGCTGCACTTCAGATCACAGA
TGTGAAATTGCAGGATGCAGGGGTGTACCGCTGCATGATCAGCTATGGTGGTGCCGACTACAAGCGAA
TTACTGTGAAAGTCAATGCCCCATACAACAAAATCAACCAAAGAATTTTGGTTGTGGATCCAGTCACC
TCTGAACATGAACTGACATGTCAGGCTGAGGGCTACCCCAAGGCCGAAGTCATCTGGACAAGCAGTGA
CCATCAAGTCCTGAGTGGTAAGACCACCACCACCAATTCCAAGAGAGAGGAGAAGCTTTTCAATGTGA
CCAGCACACTGAGAATCAACACAACAACTAATGAGATTTTCTACTGCACTTTTAGGAGATTAGATCCT
GAGGAAAACCATACAGCTGAATTGGTCATCCCAGAACTACCTCTGGCACATCCTCCAAATGAAAGGAC
TCACTTGGTAATTCTGGGAGCCATCTTATTATGCCTTGGTGTAGCACTGACATTCATCTTCCGTTTAA
GAAAAGGGAGAATGATGGATGTGAAAAAATGTGGCATCCAAGATACAAACTCAAAGAAGCAAAGTGAT
ACACATTTGGAGGAGACGTAATCCAGCATTGGAACTTCTGATCTTCAAGCAGGGATTCTCAACCTGTG
GTTTAGGGGTTCATCGGGGCTGAGCGTGACAAGAGGAAGGAATGGACCCGTGGGATGCAGGCAATGTG
GGACTTAAAAGGCCCAAGCACTGAAAATGGAACCTGGCGAAAGCAGAGGAGGAGAATGAAGAAAGATG
GAGTCAAACAGGGAGCCTGGAGGGAGACCTTGATACTTTCAAATGCCTGAGGGGCTCATCGACGCCTG
TGACAGGGAGAAAGGATACTTCTGAACAAGGAGCCTCCAAGCAAATCATCCATTGCTCATCCTAGGAA
GACGGGTTGAGAATCCCTAATTTGAGGGTCAGTTCCTGCAGAAGTGCCCTTTGCCTCCACTCAATGCC
TCAATTTGTTTTCTGCATGACTGAGAGTCTCAGTGTTGGAACGGGACAGTATTTATGTATGAGTTTTT
CCTATTTATTTTGAGTCTGTGAGGTCTTCTTGTCATGTGAGTGTGGTTGTGAATGATTTCTTTTGAAC
ATATATTGTAGTAGATGTTACAATTTTGTCGCCAAACTAAACTTGCTGCTTAATGATTTGCTCACATC
TAGTAAAACATGGAGTATTTGTAAGGTGCTTGGTCTCCTCTATAACTACAAGTATACATTGGAAGCAT
AAAGATCAAACCGTTGGTTGCATAGGATGTCACCTTTATTTAACCCATTAATACTCTGGTTGACCTAA
TCTTATTCTCAGACCTCAAGTGTCTGTGCAGTATCTGTTCCATTTAAATATCAGCTTTACAATTATGT
GGTAGCCTACACACATAATCTCATTTCATCGCTGTAACCACCCTGTTGTGATAACCACTATTATTTTA
CCCATCGTACAGCTGAGGAAGCAAACAGATTAAGTAACTTGCCCAAACCAGTAAATAGCAGACCTCAG
ACTGCCACCCACTGTCCTTTTATAATACAATTTACAGCTATATTTTACTTTAAGCAATTCTTTTATTC
AAAAACCATTTATTAAGTGCCCTTGCAATATCAATCGCTGTGCCAGGCATTGAATCTACAGATGTGAG
CAAGACAAAGTACCTGTCCTCAAGGAGCTCATAGTATAATCAGGAGATTAACAAGAAAATGTATTATT
ACAATTTAGTCCAGTGTCATAGCATAAGGATGATGCGAGGGGAAAACCCGAGCAGTGTTGCCAAGAGC
AGGAAATAGGCCAATGTGGTCTGGGACGGTTGGATATACTTAAAGATCTTAATAATCAGAGTAATTTT
CATTTACAAAGAGAGGTCGGTACTTAAAATAACCCTGAAAAATAACACTGGAATTCCTTTTCTAGCAT
TATATTTATTCCTGATTTGCCTTTGCCATATAATCTAATGCTTGTTTATATAGTGTCTGGTATTGTTT
AACAGTTCTGTCTTTTCTATTTAAATGCCACTAAATTTTAAATTCATACCTTTCCATGATTCAAAATT
CAAAAGATCCCATGGGAGATGGTTGGAAAATCTCCACTTCATCCTCCAAGCCATTCAAGTTTCCTTTC
CAGAAGCAACTGCTACTGCCTTTCATTCATATGTTCTTCTAAAGATAGTCTACATTTGGAAATGTATG
TTAAAAGCACGTATTTTTAAAATTTTTTTCCTAAATAGTAACACATTGTATGTCTGCTGTGTACTTTC
CTATTTTTATTTATTTTAGTGTTTCTTATATAGCAGATGGAATGAATTTGAAGTTCCCAGGGCTGAGG
ATCCATGCCTTCTTTGTTTCTAAGTTATCTTTCCCATAGCTTTTCATTATCTTTCATATGATCCAGTA
TATGTTAAATATGTCCTACATATACATTTAGACAACCACCATTTGTTAAGTATTTGCTCTAGGACAGA
GTTTGGATTTGTTTATGTTTGCTCAAAAGGAGACCCATGGGCTCTCCAGGGTGCACTGAGTCAATCTA
GTCCTAAAAAGCAATCTTATTATTAACTCTGTATGACAGAATCATGTCTGGAACTTTTGTTTTCTGCT
TTCTGTCAAGTATAAACTTCACTTTGATGCTGTACTTGCAAAATCACATTTTCTTTCTGGAAATTCCG
GCAGTGTACCTTGACTGCTAGCTACCCTGTGCCAGAAAAGCCTCATTCGTTGTGCTTGAACCCTTGAA
TGCCACCAGCTGTCATCACTACACAGCCCTCCTAAGAGGCTTCCTGGAGGTTTCGAGATTCAGATGCC
CTGGGAGATCCCAGAGTTTCCTTTCCCTCTTGGCCATATTCTGGTGTCAATGACAAGGAGTACCTTGG
CTTTGCCACATGTCAAGGCTGAAGAAACAGTGTCTCCAACAGAGCTCCTTGTGTTATCTGTTTGTACA
TGTGCATTTGTACAGTAATTGGTGTGACAGTGTTCTTTGTGTGAATTACAGGCAAGAATTGTGGCTGA
GCAAGGCACATAGTCTACTCAGTCTATTCCTAAGTCCTAACTCCTCCTTGTGGTGTTGGATTTGTAAG
GCACTTTATCCCTTTTGTCTCATGTTTCATCGTAAATGGCATAGGCAGAGATGATACCTAATTCTGCA
TTTGATTGTCACTTTTTGTACCTGCATTAATTTAATAAAATATTCTTATTTATTTTGTTACTTGGTAC
ACCAGCATGTCCATTTTCTTGTTTATTTTGTGTTTAATAAAATG

| | | | |
|---|---|---|---|
| NOV35g, CG56110-05 | SEQ ID NO:848 | 290 aa | MW at 33275.0 kD |
| Protein Sequence | | | |

MRIFAVFIFMTYWHLLNAFTVTVPKDLYVVEYGSNMTIECKFPVEKQLDLAALIVYWEMEDKNIIQFV
HGEEDLKVQHSSYRQRARLLKDQLSLGNAALQITDVKLQDAGVYRCMISYGGADYKRITVKVNAPYNK
INQRILVVDPVTSEHELTCQAEGYPKAEVIWTSSDHQVLSGKTTTTNSKREEKLFNVTSTLRINTTTN
EIFYCTFRRLDPEENHTAELVIPELPLAHPPNERTHLVILGAILLCLGVALTFIFRLRKGRMMDVKKC
GIQDTNSKKQSDTHLEET

| | | |
|---|---|---|
| NOV35h, CG56110-06 | SEQ ID NO:849 | 900 bp |
| DNA Sequence | ORF Start: ATG at 17 | ORF Stop: at 866 |

CACCAGATCTCCCACCATGAGGATATTTGCTGTCTTTATATTCATGACCTACTGGCATTTGCTGAACG
CATTTACTGTCACGGTTCCCAAGGACCTATATGTGGTAGAGTATGGTAGCAATATGACAATTGAATGC
AAATTCCCAGTAGAAAAACAATTAGACCTGGCTGCACTAATTGTCTATTGGGAAATGGAGGATAAGAA
CATTATTCAATTTGTGCATGGAGAGGAAGACCTGAAGGTTCAGCATAGTAGCTACAGACAGAGGGCCC
GGCTGTTGAAGGACCAGCTCTCCCTGGGAAATGCTGCACTTCAGATCACAGATGTGAAATTGCAGGAT
GCAGGGGTGTACCGCTGCATGATCAGCTATGGTGGTGCCGACTACAAGCGAATTACTGTGAAAGTCAA
TGCCCCATACAACAAAATCAACCAAAGAATTTTGGTTGTGGATCCAGTCACCTCTGAACATGAACTGA
CATGTCAGGCTGAGGGCTACCCCAAGGCCGAAGTCATCTGGACAAGCAGTGACCATCAAGTCCTGAGT
GGTAAGACCACCACCACCAATTCCAAGAGAGAGGAGAAGCTTTTCAATGTGACCAGCACACTGAGAAT
CAACACAACAACTAATGAGATTTTCTACTGCACTTTTAGGAGATTAGATCCTGAGGAAAACCATACAG
CTGAATTGGTCATCCCAGAACTACCTCTGGCACATCCTCCAAATGAAAGGACTCACTTGGTAATTCTG
GGAGCCATCTTATTATGCCTTGGTGTAGCACTGACATTCATCTTCCGTTTAAGAAAAGGGAGAATGAT
GGATGTGAAAAAATGTGGCATCCAAGATACAAACTCAAAGAAGCAAAGTGATACACATTTGGAGGAGA
CGTAAAGTCGACGGCC

| | | | |
|---|---|---|---|
| NOV35h, CG56110-06 | SEQ ID NO:850 | 283 aa | MW at 32449.2 kD |
| Protein Sequence | | | |

MRIFAVFIFMTYWHLLNAFTVTVPKDLYVVEYGSNMTIECKFPVEKQLDLAALIVYWEMEDKNIIQFV
HGEEDLKVQHSSYRQRARLLKDQLSLGNAALQITDVKLQDAGVYRCMISYGGADYKRITVKVNAPYNK
INQRILVVDPVTSEHELTCQAEGYPKAEVIWTSSDHQVLSGKTTTTNSKREEKLFNVTSTLRINTTTN
EIFYCTFRRLDPEENHTAELVIPELPLAHPPNERTHLVILGAILLCLGVALTFIFRLRKGRMMDVKKC
GIQDTNSKKQS

A ClustalW comparison of the above protein sequences yields the following sequence alignment shown in Table 35B.

TABLE 35B
Comparison of the NOV35 protein sequences.

| | | |
|---|---|---|
| NOV35a | -----MRIFAVFIFMTYWHLLNAFTVTVPKDLYVVEYGSNMTIECKFPVEKQLDLAALIV | (SEQ ID NO:836) |
| NOV35b | --------------MTYWHLLNAFTVTVPKDLYVVEYGSNMTIECKFPVEKQLDLAALIV | (SEQ ID NO:838) |
| NOV35c | TRSPTMRIFAVFIFMTYWHLLNAFTVTVPKDLYVVEYGSNMTIECKFPVEKQLDLAALIV | (SEQ ID NO:840) |
| NOV35d | -----MRIFAVFIFMTYWHLLNAFTVTVPKDLYVVEYGSNMTIECKFPVEKQLDLAALIV | (SEQ ID NO:842) |
| NOV35e | -----------------------FTVTVPKDLYVVEYGSNMTIECKFPVEKQLDLAALIV | (SEQ ID NO:844) |
| NOV35f | -----MRIFAVFIFMTYWHLLNAFTVTVPKDLYVVEYGSNMTIECKFPVEKQLDLAALIV | (SEQ ID NO:846) |
| NOV35g | -----MRIFAVFIFMTYWHLLNAFTVTVPKDLYVVEYGSNMTIECKFPVEKQLDLAALIV | (SEQ ID NO:848) |
| NOV35h | -----MRIFAVFIFMTYWHLLNAFTVTVPKDLYVVEYGSNMTIECKFPVEKQLDLAALIV | (SEQ ID NO:850) |
| NOV35a | YWEMEDKNIIQFVHGEEDLKVQHSSYRQRARLLKDQLSLGNAALQITDVKLQDAGVYRCM | |
| NOV35b | YWEMEDKNIIQFVHGEEDLKVQHSSYRQRARLLKDQLSLGNAALQITDVKLQDAGVYRCM | |
| NOV35c | YWEMEDKNIIQFVHGEEDLKVQHSSYRQRARLLKDQLSLGNAALQITDVKLQDAGVYRCM | |
| NOV35d | YWEMEDKNIIQFVHGEEDLKVQHSSYRQRARLLKDQLSLGNAALQITDVKLQDAGVYRCM | |
| NOV35e | YWEMEDKNIIQFVHGEEDLKVQHSSYRQRARLLKDQLSLGNAALQITDVKLQDAGVYRCM | |
| NOV35f | YWEMEDKNIIQFVHGEEDLKVQHSSYRQRARLLKDQLSLGNAALQITDVKLQDAGVYRCM | |
| NOV35g | YWEMEDKNIIQFVHGEEDLKVQHSSYRQRARLLKDQLSLGNAALQITDVKLQDAGVYRCM | |
| NOV35h | YWEMEDKNIIQFVHGEEDLKVQHSSYRQRARLLKDQLSLGNAALQITDVKLQDAGVYRCM | |
| NOV35a | ISYGGADYKRITVKVNAPYNKINQRILVVDPVTSEHELTCQAEGYPKAEVIWTSSDHQVL | |
| NOV35b | ISYGGADYKRITVKVNAPYNKINQRILVVDPVTSEHELTCQAEGYPKAEVIWTSSDHQVL | |
| NOV35c | ISYGGADYKRITVKVNAPYNKINQRILVVDPVTSEHELTCQAEGYPKAEVIWTSSDHQVL | |
| NOV35d | ISYGGADYKRITVKVNAPYNKINQRILVVDPVTSEHELTCQAEGYPKAEVIWTSSDHQVL | |
| NOV35e | ISYGGADYKRITVKVNAPYNKINQRILVVDPVTSEHELTCQAEGYPKAEVIWTSSDHQVL | |
| NOV35f | ISYGGADYKRITVKVNAPYNKINQRILVVDPVTSEHELTCQAEGYPKAEVIWTSSDHQVL | |
| NOV35g | ISYGGADYKRITVKVNAPYNKINQRILVVDPVTSEHELTCQAEGYPKAEVIWTSSDHQVL | |
| NOV35h | ISYGGADYKRITVKVNAPYNKINQRILVVDPVTSEHELTCQAEGYPKAEVIWTSSDHQVL | |
| NOV35a | SGKTTTTNSKREEKLFNVTSTLRINTTTNEIFYCTFRRLDPEENHTAELVIPELPLAHPP | |
| NOV35b | SGD--------------------------------------------------------- | |
| NOV35c | SGKTTTTNSKREEKLFNVTSTLRINTTTNEIFYCTFRRLDPEENHTAELVIPELPLAHPP | |
| NOV35d | SGKTTTTNSKREEKLFNVTSTLRINTTTNEIFYCTFRRLDPEENHTAELVIPELPLAHPP | |
| NOV35e | SGKTTTTNSKREEKLFNVTSTLRINTTTNEIFYCTFRRLDPEENHTAELVIPELPLAHPP | |
| NOV35f | SGD--------------------------------------------------------- | |
| NOV35g | SGKTTTTNSKREEKLFNVTSTLRINTTTNEIFYCTFRRLDPEENHTAELVIPELPLAHPP | |
| NOV35h | SGKTTTTNSKREEKLFNVTSTLRINTTTNEIFYCTFRRLDPEENHTAELVIPELPLAHPP | |
| NOV35a | NERTHLVILGAILLCLGVALTFIFRLRKGRMMDVKKCGIQDTNSKKQSDTHLEET | |
| NOV35b | ------------------------------------------------------- | |
| NOV35c | NERTHLVILGAILLCLGVALTFIFRLRKGRMMDVKKCGIQDTNSKKQSDTHLEET | |
| NOV35d | NERTHLVILGAILLCLGVALTFIFRLRKGRMMDVKKCGIQDTNSKKQSDTHLEET | |
| NOV35e | NERTH-------------------------------------------------- | |
| NOV35f | ------------------------------------------------------- | |
| NOV35g | NERTHLVILGAILLCLGVALTFIFRLRKGRMMDVKKCGIQDTNSKKQSDTHLEET | |
| NOV35h | NERTHLVILGAILLCLGVALTFIFRLRKGRMMDVKKCGIQDTNSKKQS------- | |

Further analysis of the NOV35a protein yielded the following properties shown in Table 35C.

TABLE 35C
Protein Sequence Properties NOV35a

| | |
|---|---|
| SignalP analysis: | Cleavage site between residues 19 and 20 |
| PSORT II analysis: | |

| PSG: | a new signal peptide prediction method | |
|---|---|---|
| | N-region: | length 2; pos. chg 1; neg. chg 0 |
| | H-region: | length 22; peak value 13.04 |
| | PSG score: | 8.64 |

| | |
|---|---|
| GvH: | von Heijne's method for signal seq. recognition |
| | GvH score (threshold: −2.1): −3.72 |
| | possible cleavage site: between 18 and 19 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 2 | | |
| | Number of TMS(s) for threshold 0.5: 1 | | |
| | INTEGRAL | Likelihood = | Transmembrane 242-258 |
| | | −11.36 | |

| | |
|---|---|
| PERIPHERAL | Likelihood = 7.90 (at 40) |

| | |
|---|---|
| | ALOM score: −11.36 (number of TMSs: 1) |
| MTOP: | Prediction of membrane topology (Hartmann et al.) |
| | Center position for calculation: 249 |
| | Charge difference: 5.0 C(5.0)-N(0.0) |
| | C > N: C-terminal side will be inside |

>>>Caution: Inconsistent mtop result with signal peptide
>>> Single TMS is located near the C-terminus
>>> membrane topology: type Nt (cytoplasmic tail 1 to 241)

| MITDISC: | discrimination of mitochondrial targeting seq | | | |
|---|---|---|---|---|
| | R content: | 1 | Hyd Moment(75): | 5.40 |
| | Hyd Moment(95): | 9.37 | G content: | 0 |
| | D/E content: | 1 | S/T content: | 3 |

| | |
|---|---|
| | Score: −2.89 |
| Gavel: | prediction of cleavage sites for mitochondrial preseq |
| | R-2 motif at 12 MRI\|FA |

NUCDISC: discrimination of nuclear localization signals

| | |
|---|---|
| | pat4: none |
| | pat7: none |
| | bipartite: none |
| | content of basic residues: 11.7% |
| | NLS Score: −0.47 |
| KDEL: | ER retention motif in the C-terminus: none |

ER Membrane Retention Signals:

XXRR-like motif in the N-terminus: RIFA

none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: found

LL at 15
LL at 87

checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| | total: 0 residues |

Final Results (k = 9/23):

```
30.4%: nuclear
26.1%: cytoplasmic
13.0%: Golgi
13.0%: endoplasmic reticulum
  8.7%: mitochondrial
  4.3%: vesicles of secretory system
  4.3%: peroxisomal
```

>> prediction for CG56110-03 is nuc (k = 23)

A search of the NOV35a protein against the Geneseq database, a proprietary database that contains sequences published in patents and patent publication, yielded several homologous proteins shown in Table 35D.

TABLE 35D
Geneseq Results for NOV35a

| | | NOV35a | Identities/ | |
|---|---|---|---|---|
| | | Residues/ | Similarities for | |
| Geneseq | Protein/Organism/Length | Match | the Matched | Expect |
| Identifier | [Patent #,Date] | Residues | Region | Value |
| AAE33390 | Human membrane B7-4 (B7-4M) | 1 . . . 290 | 290/290 (100%) | e−169 |
| | protein - Homo sapiens, 290 aa. | 1 . . . 290 | 290/290 (100%) | |
| | [WO200279499-A1, 10-OCT-2002] | | | |
| ABJ18518 | Human PD-L1M protein - Homo | 1 . . . 290 | 290/290 (100%) | e−169 |
| | sapiens, 290 aa. [WO200278731-A1, | 1 . . . 290 | 290/290 (100%) | |
| | 10-OCT-2002] | | | |
| AAE16964 | Human PD-1 ligand (PD-L1) - Homo | 1 . . . 290 | 290/290 (100%) | e−169 |
| | sapiens, 290 aa. [WO200200730-A2, | 1 . . . 290 | 290/290 (100%) | |
| | 03-JAN-2002] | | | |
| AAU81007 | B7-related protein, BSL1 - Homo | 1 . . . 290 | 290/290 (100%) | e−169 |
| | sapiens, 290 aa. [WO200194413-A2, | 1 . . . 290 | 290/290 (100%) | |
| | 13-DEC-2001] | | | |
| ABG64016 | Human albumin fusion protein #691 - | 1 . . . 290 | 290/290 (100%) | e−169 |
| | Homo sapiens, 290 aa. | 1 . . . 290 | 290/290 (100%) | |
| | [WO200177137-A1, 18-OCT-2001] | | | |

In a BLAST search of public sequence databases, the NOV35a protein was found to have homology to the proteins shown in the BLASTP data in Table 35E.

TABLE 35E
Public BLASTP Results for NOV35a

| | | NOV35a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9NZQ7 | B7-H1 (PD-1-ligand precursor) - | 1 . . . 290 | 290/290 (100%) | e−168 |
| | Homo sapiens (Human), 290 aa. | 1 . . . 290 | 290/290 (100%) | |
| CAC34696 | Sequence 1 from Patent | 1 . . . 227 | 227/227 (100%) | e−130 |
| | WO0114557 - Homo sapiens | 1 . . . 227 | 227/227 (100%) | |
| | (Human), 245 aa. | | | |
| Q9EP73 | PD-1-ligand precursor (B7-H1 | 1 . . . 290 | 202/291 (69%) | e−114 |
| | protein) - Mus musculus (Mouse), | 1 . . . 290 | 236/291 (80%) | |
| | 290 aa. | | | |
| Q9NUZ5 | Hypothetical protein FLJ11032 - | 115 . . . 290 | 176/176 (100%) | 7e−99 |
| | Homo sapiens (Human), 176 aa. | 1 . . . 176 | 176/176 (100%) | |
| Q9BQ51 | Butyrophilin precursor B7-DC (PD- | 19 . . . 240 | 90/223 (40%) | 2e−35 |
| | 1-ligand 2 protein) - Homo sapiens | 21 . . . 218 | 123/223 (54%) | |
| | (Human), 273 aa. | | | |

PFam analysis predicts that the NOV35a protein contains the domains shown in the Table 35F.

TABLE 35F
Domain Analysis of NOV35a

| | | Identities/ | |
|---|---|---|---|
| Pfam | NOV35a | Similarities for | Expect |
| Domain | Match Region | the Matched Region | Value |
| ig | 33 . . . 116 | 13/85 (15%) | 0.13 |
| | | 54/85 (64%) | |

### Example 36

The NOV36 clone was analyzed, and the nucleotide and encoded polypeptide sequences are shown in Table 36A.

TABLE 36A
NOV36 Sequence Analysis

| | | |
|---|---|---|
| NOV36a, CG56383-02 | SEQ ID NO:851 | 2715 bp |
| DNA Sequence | ORF Start: ATG at 34 | ORF Stop: TAA at 2566 |

TGCCTGCACTGCAAGTGTCCCCAGGAGGAGCACATGGTGACAGTGATGCCGCTGGAGATGGAGAAGAC
CATCAGCAAACTCATGTTTGACTTTCAGAGGAACTCGACCTCAGATGATGACTCAGGCTGTGCTTTGG
AAGAGTATGCCTGGGTCCCGCCGGGTCTGAAGCCTGAACAGGTACACCAGTACTATAGCTGTCTCCCA
GAAGAGAAAGTCCCTTATGTCAACAGTCCTGGAGAGAAACTGCGAATCAAGCAGCTACTACACCAGCT
GCCGCCACATGACAATGAGGTAAGGTATTGCAACTCCCTGGATGAGGAAGAGAAGAGGGAGCTGAAGC
TTTTCAGCAGCCAGAGGAAACGCGAAAACTTGGGCCGCGGGAATGTCAGGCCTTTCCCAGTCACCATG
ACAGGAGCTATTTGTGAACAGTGCGGAGGCCAGATCAATGGTGGAGACATCGCTGTGTTTGCGTCACG
CGCTGGCCACGGCGTTTGCTGGCACCCGCCGTGCTTCGTATGCACTGTCTGCAATGAGCTCCTGGTGG
ATCTGATCTACTTTTACCAAGATGGGAAGATATACTGTGGCAGGCACCATGCTGAGTGCCTGAAGCCG
CGCTGTGCTGCCTGCGATGAGATCATCTTTGCAGATGAATGCACAGAAGCTGAGGGGCGACACTGGCA
CATGAAACACTTTTGCTGCTTCGAGTGTGAGACAGTGCTGGGCGGCCAGCGCTACATCATGAAGGAGG
GAAGACCCTACTGTTGCCACTGCTTCGAGTCCTTGTATGCAGAATATTGTGACACCTGTGCCCAACAT
ATAGGTATCGACCAAGGTCAAATGACCTATGATGGCCAACACTGGCATGCCACTGAGACCTGTTTCTG
CTGTGCTCACTGCAAGAAATCCCTCCTGGGGCGGCCATTCCTCCCGAAGCAGGGCCAGATATTCTGCT
CACGGGCCTGCAGTGCTGGGGAAGACCCCAATGGTTCTGACTCCTCTGATTCCGCCTTCCAGAACGCC
AGGGCCAAGGAGTCCCGGCGCAGTGCCAAAATTGGCAAGAACAAGGGCAAGACGGAGGAGCCCATGCT
GAACCAGCACAGCCAGCTGCAAGTGAGTTCTAACCGGCTGTCAGCCGACGTAGACCCCCTGTCACTGC
AGATGGACATGCTCAGCCTGTCCAGCCAGACACCCAGCCTCAACCGGGACCCCATCTGGAGGAGCCGG
GAAGAGCCCTACCATTATGGGAACAAGATGGAGCAGAACCAGACCCAGAGCCCTCTGCAGCTCCTCAG
CCAGTGCAACATCAGAACTTCCTACAGTCCAGGAGGGCAAGGGGCTGGGGCCCAGCCCGAAATGTGGG
GCAAGCACTTCAGCAACCCCAAAAGGAGCTCGTCACTGGCCATGACAGGACATGCTGGCAGCTTCATC
AAGGAATGCCGAGAAGACTATTACCCGGGGAGGCTGAGATCTCAGGAGAGCTACAGTGATATGTCTAG
TCAGAGTTTCAGTGAGACCCGAGGCAGCATCCAAGTCCCCAAATATGAGGAGGAAGAGGAAGAGGAAG
GGGGCTTGTCCACTCAGCAGTGTCGGACCCGTCATCCCATCAGTTCCCTGAAATACACAGAGGACATG
ACGCCCACAGAGCAGACCCCTCGGGGCTCCATGGAATCCCTGGCCCTGTCTAATGCAACAGGTCTCTC
TGCTGATGGTGGTGCCAAGCGCCAGGAGCACCTATCCCGATTTTCCATGCCTGACCTCAGCAAAGACT
CTGGAATGAATGTGTCTGAGAAGCTGAGCAACATGGGCACTCTTAACTCGTCCATGCAGTTCCGGAGC
GCAGAGTCAGTTCGCAGCCTGCTCTCTGCCCAGCAGTACCAGGAGATGGAGGGAAACCTCCACCAGCT
CAGCAACCCCATTGGCTACAGAGACCTGCAGTCCCACGGAAGGATGCATCAGAGCTTTGATTTTGATG
GAGGGATGGCGGGCAGCAAGCTGCCAGGGCAGGAGGGCGTGAGGATCCAGCCCATGAGTGAACGCACC
CGGAGAAGAGCTACTTCACGCGACGACAACCGCCGTTTCCGACCTCACAGGTCCAGGCGTTCCCGACG
CTCTCGCTCCGACAACGCCCTCCACCTGGCCAGCGAACGCGAGGCCATCTCCCGGTTAAAAGATAGGC
CCCCTCTGAGAGCCAGGGAGGACTATGACCAATTTATGCGCCAGCGGAGCTTCCAGGAGAGCATGGGG
CATGGGTCCCGGAGGGACCTGTACGGCCAGTGCCCTAGGACTGTGTCGGACCTGGCTTTGCAGAATGC
CTTTGGGGACCGCTGGGGACCCTACTTCGCCGAGTATGATTGGTGTTCCACCTGCTCCTCCTCTTCAG
AGTCTGACAACGAGGGCTATTTCCTAGGAGAACCCATCCCCCAGCCAGCGCGCCTGCGATACGTCACA
AGCGATGAGCTGCTGCACAAATACAGCTCCTACGGCCTCCCCAAATCTTCCACATTAGGTGGCAGAGG
ACAGTTGCACAGCAGGAAAAGACAGAAGAGCAAAAACTGTATCATTTCTTAATATGATTGGGATCAGG
GAATGGGAGAAGATGGGAGCTAAGAATGTAAAGTCAGAAACTTGCACTGTTTTAAATGTTAAAGCGCT
TTTGGGGGTGGCTTATGGGGGAGAAAAGGGAAAATGCTGTCAGTAGATGGAGGCAAGGTTACA

| | | | |
|---|---|---|---|
| NOV36a, CG56383-02 | SEQ ID NO:852 | 844 aa | MW at 95613.8 kD |
| Protein Sequence | | | |

MVTVMPLEMEKTISKLMFDFQRNSTSDDDSGCALEEYAWVPPGLKPEQVHQYYSCLPEEKVPYVNSPG
EKLRIKQLLHQLPPHDNEVRYCNSLDEEEKRELKLFSSQRKRENLGRGNVRPFPVTMTGAICEQCGGQ
INGGDIAVFASRAGHGVCWHPPCFVCTVCNELLVDLIYFYQDGKIYCGRHHAECLKPRCAACDEIIFA
DECTEAEGRHWHMKHFCCFECETVLGGQRYIMKEGRPYCCHCFESLYAEYCDTCAQHIGIDQGQMTYD
GQHWHATETCFCCAHCKKSLLGRPFLPKQGQIFCSRACSAGEDPNGSDSSDSAFQNARAKESRRSAKI
GKNKGKTEEPMLNQHSQLQVSSNRLSADVDPLSLQMDMLSLSSQTPSLNRDPIWRSREEPYHYGNKME
QNQTQSPLQLLSQCNIRTSYSPGGQGAGAQPEMWGKHFSNPKRSSSLAMTGHAGSFIKECREDYYPGR
LRSQESYSDMSSQSFSETRGSIQVPKYEEEEEEEGGLSTQQCRTRHPISSLKYTEDMTPTEQTPRGSM
ESLALSNATGLSADGGAKRQEHLSRFSMPDLSKDSGMINSEKLSNMGTLNSSMQFRSAESVRSLLSAQ
QYQEMEGNLHQLSNPIGURDLQSHGRMHQSFDFDGGMAGSKLPGQEGVRIQPMSERTRRRATSRDDNR
RFRPHRSRRSRRSRSDNALHLASEREAISRLKDRPPLRAREDYDQFMRQRSFQESMGHGSRRDLYGQC
PRTVSDLALQNAFGDRWGPYFAEYDWCSTCSSSSESDNEGYFLGEPIPQPARLRYVTSDELLHKYSSY
GLPKSSTLGGRGQLHSRKRQKSKNCIIS

| | | |
|---|---|---|
| NOV36b, CG56383-01 | SEQ ID NO:853 | 1190 bp |
| DNA Sequence | ORF Start: ATG at 54 | ORF Stop: TGA at 1014 |

TTATTGACAGTTTATCCTGCCGCACCTGGAATCCTGAGACAAACCAAGGTGCTATGTGTTTCACGTCC
CAGTGCAGAGCTCTGAGCAGCTCATCAGCCTCTCCAATGTCTCTCATTTTTTTAGGTATCGACCAAGG
TCAAATGACCTATGACGGCCAACACTGGCATGCCACTGAGACCTGTTTCTGCTGTGCTCACTGCAAGA
AATCCCTCCTGGGGCGGCCATTCCTCCCGAAGCAGGGCCAGATATTCTGCTCACGGGCCTGCAGTGCT
GGGGAAGACCCCAATGGTTCTGACTCCTCTGATTCCGCCTTCCAGAACGCCAGGGCCAAGGAGTCCCG
GCGCAGTGCCAAAATTGGCAAGAACAAGGGCAAGACGGAGGAGCCCATGCTGAACCAGCACAGCCAGC
TGCAAGTGAGTTCTAACCGGCTGTCAGCCGACGTAGACCCCCTGTCACTGCAGATGGACATGCTCAGC
CTGTCCAGCCAGACACCCAGCCTCAACCGGGACCCCATCTGGAGGAGCCGGGAAGAGCCCTACCATTA
AGGGAACAAGATGGAGCAGAACCAGACCCAGAGCCCTCTGCAGCTCCTCAGCCAGTGCAACATCAGAA
CTTCCTACAGTCCAGGAGGGCAAGGGGCTGGGGCCCAGCCCGAAATGTGGGGCAAGCACTTCAGCAAC
CCCAAAAGGAGCACGTCACTGGCCATGACAGGACATGCTGGCAGCTTCATCAAGGAATGCCGAGAAGA
CTATTACCCGGGGAGGCTGAGATCTCAGGAGAGCTACAGTGATATGTCTAGTCAGAGTTTCAGTGAGA
CCCGAGGCAGCATCCAAGTCCCCAAATATGAGGAGGAAGAGGAAGAGGAAGGGGGCTTGTCCACTCAG
CAGTGTCGGACCCGTCATCCCATCAGTTCCCTGAAATACACAGAGGACATGACGCCCACAGAGCAGAC
CCCTCGGGGCTCCATGGAATCCCTGGCCCTGTCTAATGCAACAGGTAGGTTCTGTTCACCTTGAAAAC
AGATAGAAGGGGGTAGTCTCTGGGTGACTGGATGCTGGTCCCCAGGAATTTTTTTTTTTTTGAAATGG
AGTCTCGCTCTGTCCCCCAGGCTGGAGTGCAGTGGCACGATCTCCGCTCACTGCAAGCTCCACCTCCC
GGGTTCACGCCATTCTCCTGCCTCAGCCTCACGA

| | | | |
|---|---|---|---|
| NOV36b, CG56383-01 | SEQ ID NO:854 | 320 aa | MW at 35524.0 kD |
| Protein Sequence | | | |

MCFTSQCRALSSSSASPMSLTFLGIDQGQMTYDGQHWHATETCFCCAHCKKSLLGRPFLPKQGQIFCS
EACSAGEDPNGSDSSDSAFQNARAXESRRSAKIGKNKGKTEEPMLNQHSQLQVSSNRLSADVDPLSLQ
MDMLSLSSQTPSLNRDPIWRSREEPYHYGNKMEQNQTQSPLQLLSQCNIRTSYSPGGQGAGAQPEMWG
KHFSNPKRSTSLAMTGHAGSFIKECREDYYPGRLRSQESYSDMSSQSFSETRGSIQVPKYEEEEEEEG
GLSTQQCRTRHPISSLKYTEDMTPTEQTPRGSMESLALSNATGRFCSP

| | | |
|---|---|---|
| NOV36c, SNP13382513 of | SEQ ID NO:855 | 2715 bp |
| CG56383-02, DNA Sequence | ORF Start: ATG at 34 | ORF Stop: TAA at 2566 |
| | SNP Pos: 989 | SNP Change: C to T |

TGCCTGCACTGCAAGTGTCCCCAGGAGGAGCACATGGTGACAGTGATGCCGCTGGAGATGGAGAAGAC
CATCAGCAAACTCATGTTTGACTTTCAGAGGAACTCGACCTCAGATGATGACTCAGGCTGTGCTTTGG
AAGAGTATGCCTGGGTCCCGCCGGGTCTGAAGCCTGAACAGGTACACCAGTACTATAGCTGTCTCCCA
GAAGAGAAAGTCCCTTATGTCAACAGTCCTGGAGAGAAACTGCGAATCAAGCAGCTACTACACCAGCT
GCCGCCACATGACAATGAGGTAAGGTATTGCAACTCCCTGGATGAGGAAGAGAAGAGGGAGCTGAAGC
TTTTCAGCAGCCAGAGGAAACGCGAAAACTTGGGCCGCGGGAATGTCAGGCCTTTCCCAGTCACCATG
ACAGGAGCTATTTGTGAACAGTGCGGAGGCCAGATCAATGGTGGAGACATCGCTGTGTTTGCGTCACG
CGCTGGCCACGGCGTTTGCTGGCACCCGCCGTGCTTCGTATGCACTGTCTGCAATGAGCTCCTGGTGG
ATCTGATCTACTTTTACCAAGATGGGAAGATATACTGTGGCAGGCACCATGCTGAGTGCCTGAAGCCG
CGCTGTGCTGCCTGCGATGAGATCATCTTTGCAGATGAATGCACAGAAGCTGAGGGGCGACACTGGCA
CATGAAACACTTTTGCTGCTTCGAGTGTGAGACAGTGCTGGGCGGCCAGCGCTACATCATGAAGGAGG
GAAGACCCTACTGTTGCCACTGCTTCGAGTCCTTGTATGCAGAATATTGTGACACCTGTGCCCAACAT
ATAGGTATCGACCAAGGTCAAATGACCTATGATGGCCAACACTGGCATGCCACTGAGACCTGTTTCTG
CTGTGCTCACTGCAAGAAATCCCTCCTGGGGCGGCCATTCCTCCCGAAGCAGGGCCAGATATTCTGCT
CACGGGCCTGCAGTGCTGGGGAAGACCCCAATGGTTTTGACTCCTCTGATTCCGCCTTCCAGAACGCC
AGGGCCAAGGAGTCCCGGCGCAGTGCCAAAATTGGCAAGAACAAGGGCAAGACGGAGGAGCCCATGCT
GAACCAGCACAGCCAGCTGCAAGTGAGTTCTAACCGGCTGTCAGCCGACGTAGACCCCCTGTCACTGC
AGATGGACATGCTCAGCCTGTCCAGCCAGACACCCAGCCTCAACCGGGACCCCATCTGGAGGAGCCGG
GAAGAGCCCTACCATTATGGGAACAAGATGGAGCAGAACCAGACCCAGAGCCCTCTGCAGCTCCTCAG
CCAGTGCAACATCAGAACTTCCTACAGTCCAGGAGGGCAAGGGGCTGGGGCCCAGCCCGAAATGTGGG
GCAAGCACTTCAGCAACCCCAAAAGGAGCTCGTCACTGGCCATGACAGGACATGCTGGCAGCTTCATC
AAGGAATGCCGAGAAGACTATTACCCGGGGAGGCTGAGATCTCAGGAGAGCTACAGTGATATGTCTAG
TCAGAGTTTCAGTGAGACCCGAGGCAGCATCCAAGTCCCCAAATATGAGGAGGAAGAGGAAGAGGAAG
GGGGCTTGTCCACTCAGCAGTGTCGGACCCGTCATCCCATCAGTTCCCTGAAATACACAGAGGACATG
ACGCCCACAGAGCAGACCCCTCGGGGCTCCATGGAATCCCTGGCCCTGTCTAATGCAACAGGTCTCTC
TGCTGATGGTGGTGCCAAGCGCCAGGAGCACCTATCCCGATTTTCCATGCCTGACCTCAGCAAAGACT
CTGGAATGAATGTGTCTGAGAAGCTGAGCAACATGGGCACTCTTAACTCGTCCATGCAGTTCCGGAGC
GCAGAGTCAGTTCGCAGCCTGCTCTCTGCCCAGCAGTACCAGGAGATGGAGGGAAACCTCCACCAGCT
CAGCAACCCCATTGGCTACAGAGACCTGCAGTCCCACGGAAGGATGCATCAGAGCTTTGATTTTGATG
GAGGGATGGCGGGCAGCAAGCTGCCAGGGCAGGAGGGCGTGAGGATCCAGCCCATGAGTGAACGCACC
CGGAGAAGAGCTACTTCACGCGACGACAACCGCCGTTTCCGACCTCACAGGTCCAGGCGTTCCCGACG
CTCTCGCTCCGACAACGCCCTCCACCTGGCCAGCGAACGCGAGGCCATCTCCCGGTTAAAAGATAGGC
CCCCTCTGAGAGCCAGGGAGGACTATGACCAATTTATGCGCCAGCGGAGCTTCCAGGAGAGCATGGGG
CATGGGTCCCGGAGGGACCTGTACGGCCAGTGCCCTAGGACTGTGTCGGACCTGGCTTTGCAGAATGC
CTTTGGGGACCGCTGGGGACCCTACTTCGCCGAGTATGATTGGTGTTCCACCTGCTCCTCCTCTTCAG
AGTCTGACAACGAGGGCTATTTCCTAGGAGAACCCATCCCCCAGCCAGCGCGCCTGCGATACGTCACA
AGCGATGAGCTGCTGCACAAATACAGCTCCTACGGCCTCCCCAAATCTTCCACATTAGGTGGCAGAGG
ACAGTTGCACAGCAGGAAAAGACAGAAGAGCAAAAACTGTATCATTTCTTAATATGATTGGGATCAGG
GAATGGGAGAAGATGGGAGCTAAGAATGTAAAGTCAGAAACTTGCACTGTTTTAAATGTTAAAGCGCT
TTTGGGGGTGGCTTATGGGGGAGAAAAGGGAAAATGCTGTCAGTAGATGGAGGCAAGGTTACA

| | | | |
|---|---|---|---|
| NOV36c, SNP13382513 of | SEQ ID NO:856 | 844 aa | MW at 95673.9 kD |
| CG56383-02, Protein Sequence | SNP Pos: 319 | | SNP Change: Ser to Phe |

MVTVMPLEMEKTISKLMFDFQRNSTSDDDSGCALEEYAWVPPGLKPEQVHQYYSCLPEEKVPYVNSPG
EKLRIKQLLHQLPPHDNEVRYCNSLDEEEKRELKLFSSQRKRENLGRGNVRPFPVTMTGAICEQCGGQ
INGGDIAVFASRAGHGVCWHPPCFVCTVCNELLVDLIYFYQDGKIYCGRHHAECLKPRCAACDEIIFA
DECTEAEGRHWHMKHFCCFECETVLGGQRYIMKEGRPYCCHCFESLYAEYCDTCAQHIGIDQGQMTYD
GQHWHATETCFCCAHCKKSLLGRPFLPKQGQIFCSRACSAGEDPNGFDSSDSAFQNARAKESRRSAKI
GKNKGKTEEPMLNQHSQLQVSSNRLSADVDPLSLQMDMLSLSSQTPSLNRDPIWRSREEPYHYGNKME
QNQTQSPLQLLSQCNIRTSYSPGGQGAGAQPEMWGKHFSNPKRSSSLAMTGHAGSFIKECREDYYPGR
LRSQESYSDMSSQSFSETRGSIQVPKYEEEEEEEGGLSTQQCRTRHPISSLKYTEDMTPTEQTPRGSM
ESLALSNATGLSADGGAKRQEHLSRFSMPDLSKDSGMNVSEKLSNMGTLNSSMQFRSAESVRSLLSAQ
QYQEMEGNLHQLSNPIGYRDLQSHGRMHQSFDFDGGMAGSKLPGQEGVRIQPMSERTRRRATSRDDNR
RFRPHRSRRSRRSRSDNALHLASEREAISRLKDRPPLRAREDYDQFMRQRSFQESMGHGSRRDLYGQC
PRTVSDLALQNAFGDRWGPYFAEYDWCSTCSSSSESDNEGYFLGEPIPQPARLRYVTSDELLHKYSSY
GLPKSSTLGGRGQLHSRKRQKSKNCIIS

A ClustalW comparison of the above protein sequences yields the following sequence alignment shown in Table 36B.

TABLE 36B

Comparison of the NOV36 protein sequences.

| | |
|---|---|
| NOV36a | MVTVMPLEMEKTISKLMFDFQRNSTSDDDSGCALEEYAWVPPGLKPEQVHQYYSCLPEEK |
| NOV36b | ------------------------------------------------------------ |
| NOV36a | VPYVNSPGEKLRIKQLLHQLPPHDNEVRYCNSLDEEEKRELKLFSSQRKRENLGRGNVRP |
| NOV36b | ------------------------------------------------------------ |
| NOV36a | FPVTMTGAICEQCGGQINGGDIAVFASRAGHGVCWHPPCFVCTVCNELLVDLIYFYQDGK |
| NOV36b | ------------------------------------------------------------ |
| NOV36a | IYCGRHHAECLKPRCAACDEIIFADECTEAEGRHWHMXHFCCFECETVLGGQRYIMKEGR |
| NOV36b | -----------------------------------------------------------M |
| NOV36a | PYCCHCFESLYAEYCDTCAQHIGIDQGQMTYDGQHWHATETCFCCAHCKKSLLGRPFLPK |
| NOV36b | CFTSQCRALSSSSASPMSLIFLGIDQGQMTYDGQHWHATETCFCCAHCKKSLLGRPFLPK |
| N0V36a | QGQIFCSRACSAGEDPNGSDSSDSAFQNARAKESRRSAKTGKNKGKTEEPMLNQHSQLQV |
| NOV36b | QGQIFCSRACSAGEDPNGSDSSDSAFQNARAKESRRSAKIGKNKGKTEEPMLNQHSQLQV |
| NOV36a | SSNRLSADVDPLSLQMDMLSLSSQTPSLNRDPIWRSREEPYHYGNKMEQNQTQSPLQLLS |
| NOV36b | SSNRLSADVDPLSLQMDMLSLSSQTPSLNRDPIWRSREEPYHYGNKMEQNQTQSPLQLLS |
| NOV36a | QCNIRTSYSPGGQGAGAQPEMWGKHFSNPKRSSSLAMTGHAGSFIKECREDYYPGRLRSQ |
| NOV36b | QCNIRTSYSPGGQGAGAQPEMWGKHFSNPKRSTSLAMTGHAGSFIKECREDYYPGRLRSQ |
| NOV36a | ESYSDMSSQSFSETRGSIQVPKYEEEEEEEGGLSTQQCRTRHPISSLKYTEDMTPTEQTP |
| NOV36b | ESYSDMSSQSFSETRGSIQVPKYEEEEEEEGGLSTQQCRTRHPISSLKYTEDMTPTEQTP |
| NOV36a | RGSMESLALSNATGLSADGGAKRQEHLSRFSMPDLSKDSGMNVSEKLSNMGTLNSSMQFR |
| NOV36b | RGSMESLALSNATGRFCSP----------------------------------------- |
| NOV36a | SAESVRSLLSAQQYQEMEGNLHQLSNPIGYRDLQSHGRMHQSFDFDGGMAGSKLPGQEGV |
| NOV36b | ------------------------------------------------------------ |
| NOV36a | RIQPMSERTRRRATSRDDNRRFRPHRSRRSRRSRSDNALHLASEREAISRLKDRPPLRAR |
| NOV36b | ------------------------------------------------------------ |
| NOV36a | EDYDQFMRQRSFQESMGHGSRRDLYGQCPRTVSDLALQNAFGDRWGPYFAEYDWCSTCSS |
| NOV36b | ------------------------------------------------------------ |
| NOV36a | SSESDNEGYFLGEPIPQPARLRYVTSDELLHKYSSYGLPKSSTLGGRGQLHSRKRQKSKN |
| NOV36b | ------------------------------------------------------------ |
| NOV36a | CIIS |
| NOV36b | ---- |
| NOV36a | (SEQ ID NO: 852) |
| NOV36b | (SEQ ID NO: 854) |

Further analysis of the NOV36a protein yielded the following properties shown in Table 36C.

TABLE 36C
Protein Sequence Properties NOV36a

| | |
|---|---|
| SignalP analysis: | No Known Signal Sequence Predicted |
| PSORT II analysis: | |

| PSG: | a new signal peptide prediction method | |
|---|---|---|
| | N-region: | length 11; pos. chg 1; neg. chg 2 |
| | H-region: | length 3; peak value 0.00 |
| | PSG score: | −4.40 |

| | |
|---|---|
| GvH: | von Heijne's method for signal seq. recognition |
| | GvH score (threshold: −2.1): −10.32 |
| | possible cleavage site: between 26 and 27 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 1 | |
| | Number of TMS(s) for threshold 0.5: 0 | |
| | PERIPHERAL | Likelihood = 8.54 (at 365) |

| | ALOM score: −1.17 (number of TMSs: 0) | | | |
|---|---|---|---|---|
| MITDISC: | discrimination of mitochondrial targeting seq | | | |
| | R content: | 0 | Hyd Moment(75): | 5.58 |
| | Hyd Moment(95): | 3.29 | G content: | 0 |
| | D/E content: | 2 | S/T content: | 1 |

| | |
|---|---|
| | Score: −7.15 |
| Gavel: | prediction of cleavage sites for mitochondrial preseq |
| | cleavage site motif not found |

NUCDISC: discrimination of nuclear localization signals

| | |
|---|---|
| | pat4: none |
| | pat7: PHRSRRS (4) at 684 |
| | bipartite: none |
| | content of basic residues: 12.6% |
| | NLS Score: −0.13 |
| KDEL: | ER retention motif in the C-terminus: none |

ER Membrane Retention Signals: none
SKL: peroxisomal targeting signal in the C-terminus: none
PTS2: 2nd peroxisomal targeting signal: found
RIKQLLHQL at 72
VAC: possible vacuolar targeting motif: none
RNA-binding motif: none
Actinin-type actin-binding motif:

type 1: none
type 2: none

NMYR: N-myristoylation pattern: none
Prenylation motif: CaaX motif in the C-terminus: CIIS

if X is S, A, or M, it will be farnesylated
otherwise, it will be geranylgeranylated

memYQRL: transport motif from cell surface to Golgi: none
Tyrosines in the tail: none
Dileucine motif in the tail: none
checking 63 PROSITE DNA binding motifs:

```
Leucine zipper pattern (PS00029): *** found ***
  LQVSSNRLSADVDPLSLQMDML at 358
LIM domain signature (PS00478): *** found ***
  CEQCGGQINGGDIAVFASRAGHGVCWHPPCFV
  CTVCNELL at 130
```

checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 94.1 |
| COIL: | Lupas's algorithm to detect coiled-coil regions |
| | total: 0 residues |

Final Results (k = 9/23):

```
73.9%: nuclear
  8.7%: mitochondrial
  8.7%: plasma membrane
  8.7%: cytoplasmic
```

>> prediction for CG56383-02 is nuc (k = 23)

A search of the NOV36a protein against the Geneseq database, a proprietary database that contains sequences published in patents and patent publication, yielded several homologous proteins shown in Table 36D.

TABLE 36D
Geneseq Results for NOV36a

| | | NOV36a | Identities/ | |
|---|---|---|---|---|
| | | Residues/ | Similarities for | |
| Geneseq | Protein/Organism/Length | Match | the Matched | Expect |
| Identifier | [Patent #,Date] | Residues | Region | Value |
| ABG07025 | Novel human diagnostic protein | 49 . . . 844 | 721/807 (89%) | 0.0 |
| | #7016 - Homo sapiens, 795 aa. | 1 . . . 795 | 737/807 (90%) | |
| | [WO200175067-A2, 11-OCT-2001] | | | |
| ABB82161 | Human NOV6b protein sequence - | 1 . . . 554 | 554/554 (100%) | 0.0 |
| | Homo sapiens, 559 aa. | 1 . . . 554 | 554/554 (100%) | |
| | [WO200270660-A2, 12-SEP-2002] | | | |
| ABU11832 | Human MDDT polypeptide SEQ ID | 5 . . . 844 | 429/851 (50%) | 0.0 |
| | 779 - Homo sapiens, 839 aa. | 9 . . . 839 | 542/851 (63%) | |
| | [WO200279449-A2, 10-OCT-2002] | | | |
| ABU11569 | Human MDDT polypeptide SEQ ID | 5 . . . 844 | 429/851 (50%) | 0.0 |
| | 516 - Homo sapiens, 839 aa. | 9 . . . 839 | 542/851 (63%) | |
| | [WO200279449-A2, 10-OCT-2002] | | | |
| ABG08405 | Novel human diagnostic protein | 49 . . . 350 | 302/302 (100%) | 0.0 |
| | #8396 - Homo sapiens, 311 aa. | 1 . . . 302 | 302/302 (100%) | |
| | [WO200175067-A2, 11-OCT-2001] | | | |

In a BLAST search of public sequence databases, the NOV36a protein was found to have homology to the proteins shown in the BLASTP data in Table 36E.

TABLE 36E
Public BLASTP Results for NOV36a

| | | NOV36a | Identities/ | |
|---|---|---|---|---|
| Protein | | Residues/ | Similarities for | |
| Accession | | Match | the Matched | Expect |
| Number | Protein/Organism/Length | Residues | Portion | Value |
| AAH50793 | Similar to prickle-like 1 (Drosophila) - | 1 . . . 844 | 803/845 (95%) | 0.0 |
| | Mus musculus (Mouse), 879 aa | 35 . . . 879 | 826/845 (97%) | |
| | (fragment). | | | |
| AAP33399 | Prickle2 - Brachydanio rerio | 5 . . . 844 | 569/852 (66%) | 0.0 |
| | (Zebrafish) (Danio rerio), 840 aa. | 1 . . . 840 | 664/852 (77%) | |
| Q96MT3 | Hypothetical protein FLJ31937 - | 5 . . . 844 | 428/851 (50%) | 0.0 |
| | Homo sapiens (Human), 831 aa. | 1 . . . 831 | 541/851 (63%) | |
| Q90WV2 | LIM protein prickle b - Xenopus | 5 . . . 844 | 413/861 (47%) | 0.0 |
| | laevis (African clawed frog), 866 aa. | 35 . . . 866 | 538/861 (61%) | |
| AAP33398 | Prickle1 - Brachydanio rerio | 7 . . . 844 | 387/853 (45%) | 0.0 |
| | (Zebrafish) (Danio rerio), 793 aa. | 9 . . . 793 | 499/853 (58%) | |

PFam analysis predicts that the NOV36a protein contains the domains shown in the Table 36F.

TABLE 36F
Domain Analysis of NOV36a

| | | Identities/ | |
|---|---|---|---|
| Pfam | NOV36a | Similarities for | |
| Domain | Match Region | the Matched Region | Expect Value |
| LIM | 130 . . . 192 | 19/66 (29%) | 2.9e−11 |
| | | 43/66 (65%) | |
| LIM | 195 . . . 252 | 17/62 (27%) | 3.9e−13 |
| | | 45/62 (73%) | |
| LIM | 255 . . . 315 | 16/66 (24%) | 0.022 |
| | | 34/66 (52%) | |

### Example 37

The NOV37 clone was analyzed, and the nucleotide and encoded polypeptide sequences are shown in Table 37A.

TABLE 37A

NOV37 Sequence Analysis

| | | |
|---|---|---|
| NOV37a, CG56449-04 | SEQ ID NO: 857 | 877 bp |
| DNA Sequence | ORF Start: ATG at 25 | ORF Stop: TAG at 535 |

CCGGAGCTGCCTGTGATGCCGTGAATGGCTCCTGCCTCTGCCCCGCTGGCCGCCGGGGCCCCCGCTGT
GCCGAGACCTGCCCTGCCGGCCTGTACGGCGACAACTGTCGGCATTCCTGCCTCTGCCAGAACGGAGG
GACCTGTGACCCTGTCTCAGGCCTGCGAGCACCCCTGTCCCCCTGGCTTCCACGGGGCTGGCCGCCAG
GGGTTGTGCTGGTGTCAACATGGAGCCCCCTGCGACCCCATCAGTGGCCGATGCCTCTGCCCTGCCGG
CTTCCACGGCCACTTCTGTGAGAGGGATTGCAGAAGGGGCCAGTTTGGGCCCAGCTGCACCCTGCACT
GTGACTGCGGGGGTGGGGCTGACTGCGACCCTGTCAGTGGGCAGTGTCACTGTGTGGATGGCTACATG
GGGCCCACGTGCCGGGAAGGTGGGCCCCTCCGGCTCCCCGAGAACCCGTCCTTAGCCCAGGGCTCAGC
GGGCACACTGCCCGCCTCCAGCAGACCCACATCCCGGAGCGGTGGACCAGCGAGGCACTAGTAGAGGC
AGTCCCGTGGAGCCCGCCTCTCCAGTCCCAGCCAGAGGGGACCCTGGCCTTTGGTGACCACTGAGAAC
GACACTTCACGGGCCCAGAGCTCCTGGTACTGCCCTTCCTTTGAGGGCCGTGGAGGGCTGTGGACAGC
CCAGCAACCTGTCGCTCTTGGAGGCTGGTGTGGCCTTGAGGAGGGAAGCCTCGCATGGCCGCTGGAAG
AGAGGCGTCTCCTGGCCTGGCTCTGCAGAACCCAGGGGCACGCTCTGGGCCTGGGCTGAGGAAGTCCC
GCTCTCCCGCGGCTCTGAGTTGGACTGAGGACAGGTGTGGGCGCCAGTGTGGGTGCAGGCG

| | | | |
|---|---|---|---|
| NOV37a, CG56449-04 | | | |
| Protein Sequence | SEQ ID NO: 858 | 170 aa | MW at 17123.1kD |

MAPASAPLAAGAPAVPRPALPACTATTVGIPASARTEGPVTLSQACEHPCPPGFHGAGRQGLCWCQHG
APCDPISGRCLCPAGFHGHFCERDCRRGQFGPSCTLHCDCGGGADCDPVSGQCHCVDGYMGPTCREGG
PLRLPENPSLAQGSAGTLPASSRPTSRSGGPARH

| | | |
|---|---|---|
| NOV37b, CG56449-09 | SEQ ID NO: 859 | 5172 bp |
| DNA Sequence | ORF Start: ATG at 16 | ORF Stop: TAG at 4798 |

GCACCGGCGCGCACGATGTCGTTCCTTGAAGAGGCGAGGGCAGCGGGGCGCGCGGTGGTCCTGGCGTT
GGTGCTGCTGCTGCTCCCCGCCGTGCCCGTGGGCGCCAGCGTTCCGCCGCGGCCCCTGCTCCCGCTGC
AGCCCGGCATGCCCCACGTGTGTGCTGAGCAGGAGCTGACCCTGGTGGGCCGCCGCCAGCCGTGCGTG
CAGGCCTTAAGCCACACGGTGCCGGTGTGGAGGGCCGGCTGTGGGTGGCAGGCGTGGTGCGTGGGTCA
TGAGCGGAGAACCGTCTACTACATGGGCTACAGGCAGGTGTATACCACGGAGGCCCGGACCGTGCTCA
GGTGCTGCCGAGGGTGGACGCAGCAGCCCGACGAGGAGGGCTGCCTCTCGGCTGAATGCAGCGCCAGC
CTCTGTTTTCACGGTGGCCGTTGTGTGCCAGGCTCAGCCCAGCCGTGTCACTGTCCCCCCGGCTTCCA
GGGACCCCGCTGTCAGTATGATGTGGACGAATGCCGAACCCACAACGGTGGCTGCCAGCACCGGTGCG
TGAACACCCCAGGCTCCTACCTCTGTGAGTGCAAGCCCGGCTTCCGGCTCCACACTGACAGCAGGACC
TGCCTGGCCATTAACTCCTGCGCCCTGGGCAATGGCGGCTGCCAGCACCACTGTGTCCAGCTCACAAT
CACTCGGCATCGCTGCCAGTGCCGGCCCGGGTTCCAGCTCCAGGAGGACGGCAGGCATTGTGTCCGTA
GAAGCCCGTGTGCCAACAGGAACGGCAGCTGCATGCACAGGTGCCAGGTGGTCCCGGGGCCTCGTCAG
TGTGAGTGCCACGTGGGCTATCAGCTAGCAGCGGACGGCAAGGCCTGTGAAGATGTGGACGAATGTGC
CGCAGGGCTGGCCCAGTGTGCCCATGGCTGCCTCAACACCCAGGGGTCCTTCAAGTGCGTGTGTCACG
CGGGCTATGAGCTGGGCGCCGATGGCCGGCAGTGCTACCGTATTGAGATGGAAATCGTGAACAGCTGT
GAGGCCAACAACGGCGGCTGCTCCCATGGCTGCAGCCACACCAGTGCTGGGCCCCTGTGCACCTGTCC
CCGCGGCTACGAGCTGGACACAGATCAGAGGACCTGCATCAGATGTCGACGACTGTGCAGACAGCCCG
TGCTGCAGCAGGTGTGCACCAACAACCCTGGCGGGTACGAGTGCGGCTGCTACGCCGGCTACCGGCTC
AGTGCCGATGGCTGCGGCTGCGAGGATGTGGATGAGTGCGCCTCCAGCCGTGGCGGCTGCGAGCACCA
CTGCACCAACCTGGCCGGCTCCTTCCAGTGCTCCTGCGAGGCCGGCTACCGGCTGCACGAGGACCGTA
GGGGCTGCAGCGCCCTGGAGGAGCCGATGGTGGACCTGGACGGCGAGCTGCCTTTCGTGCGGCCCCTG
CCCCACATTGCCGTGCTCCAGGACGAGCTGCCGCAACTCTTCCAGGATGACGACGTCGGGACCGATGA
GGAAGAGGCAGAGTTGCGGGGCGAACACACGCTCACAGAGAAGTTTGTCTGCCTGGATGACTCCTTTG
GCCATGACTGCAGCTTGACCTGTGATGACTGCAGGAACGGAGGGACCTGCCTCCTGGGCCTGGATGGC
TGTGATTGCCCCGAGGGCTGGACTGGGCTCATCTGCAATGAGAGTTGTCCTCCGGACACCTTTGGGAA
GAACTGCAGCTTCTCCTGCAGCTGTCAGAATGGTGGGACCTGCGACTCTGTCACGGGGGCCTGCCGCT
GCCCCCCGGGTGTCAGTGGAACTAACTGTGAGGATGGCTGCCCCAAGGGCTACTATGGCAAGCACTGT
CGCAAGAAATGCAACTGTGCCAACCGGGGCCGGTGCCACCGCCTCTACGGGGCCTGCCTCTGCGACCC
AGGGCTCTACGGCCGCTTCTGCCACCTCGCCTGCCCGCCGTGGGCCTTTGGGCCGGGCTGCTCGGAGG
AGTGCCAGTGTGTGCAGCCCCACACGCAGTCCTGTGACAAGAGGGATGGCAGCTGCTCCTGCAAGGCT
GGCTTCCGGGGCGAGCGCTGTCAGGCAGAGTGTGAGCCGGGCTACTTTGGGCCGGGGTGCTGGCAGGC
ATGCACCTGCCCAGTGGGCGTGGCCTGTGACTCCGTGAGCGGCGAGTGTGGGAAGCGGTGTCCTGCTG
GCTTCCAGGGAGAGGACTGTGGCCAAGAGTGCCCGGTGGGGACCTTTGGCGTGAACTGCTCGAGCTCC
TGCTCCTGTGGGGGGGCCCCCTGCCACGGGGTCACGGGGCAGTGCCGGTGTCCGCCGGGGAGGACTGG
GGAAGACTGTGAGGCAGGTGAGTGTGAGGGCCTCTGGGGGCTGGGCTGCCAGGAGATCTGCCCAGCAT
GCCATAACGCTGCTCGCTGCGACCCTGAGACCGGAGCCTGCCTGTGCCTCCCTGGCTTTGTCGGCAGC
CGCTGCCAGGACTGTGAGGCAGGCTGGTATGGTCCCAGCTGCCAGACAATGTGCTCTTGTGCCAATGA
TGGGGCACTGCCACCAAGACACGGGACACTGCAGCTGTGCCCCGGGTGGACCGGCTTTAGCTGCCAGA
GAGCCTGTGATACTGGGCACTGGGGACCTGACTGCAGCCACCCCTGCAACTGCAGCGCTGGCCACGGG
AGCTGTGATGCCATCAGCGGCCTGTGTCTGTGTGAGGCTGGCTACGTGGGCCCGCGGTGCGAGCAGTC
AGAGTGTCCCCAGGGCCACTTTGGGCCCGGCTGTGAGCAGCGGTGCCAGTGTCAGCATGGAGCAGCCT
GTGACCACGTCAGCGGGGCCTGCACCTGCCCGGCCGGCTGGAGGGGCACCTTCTGCGAGCATAACTGC
CCGGCCGGCTTCTTTGGATTGGACTGTCGCAGTGCCTGCAACTGCACCGCCGGAGCTGCCTGTGATGC
CGTGAATGGCTCCTGCCTCTGCCCCGCTGGCCGCCGGGGCCCCCGCTGTGCCGAGAGTGCCTGCCCAG
CCCACACCTACGGGCACAATTGCAGCCAGGCCTGTGCCTGCTTTAACGGGGCCTCCTGTGACCCTGTC
CACGGGCAGTGCCACTGTGCCCCTGGCTGGATGGGGCCCTCCTGCCTGCAGGCCTGCCCTGCCGGCCT
GTACGGCGACAACTGTCGGCATTCCTGCCTCTGCCAGAACGGAGGGACCTGTGACCCTGTCTCAGGCC
ACTGTGCGTGCCCAGAGGGCTGGGCCGGCCTGGCCTGTGAGGTAGAGTGCCTCCCCCGGGACGTCAGA
GCTGGCTGCCGGCACAGCGGCGGTTGCCTCAACGGGGGCCTGTGTGACCCGCACACGGGCCGCTGCCT
CTGCCCAGCCGGCTGGACTGGGGACAAGTGTCAGAGCCCTGCAGCCTGTGCCAAGGAAACATTCGGGC
CTCACTGTGAGGGGCGCTGTGCCTGCCGGTGGGGAGGCCCCTGCCACCTTGCCACCGGGGCCTGCCTC
TGCCCTCCGGGGTGGCGGGGGCCTCATCTTTCTGCAGCCTGCCTGCGAAGCTGGTTTGGAGAGGCCTG
TGCCCAGCGCTGCAGCTGCCCGCCTGGCGCTGCCTGCCACCACGTCACTGGGGCCTAACGCTGTCCCC
CTGGCTTCACTGGCTCCGGCTGCGAGCAGGCCTGCCCACCCGGCAGCTTTGGGGAGGACTGTGCGCAG
ATGTGCCAGTGTCCCGGTGAGAACCCGGCCTGCCACCCTGCCACCGGGACCTGCTCATGTGCTGCTGG
CTACCACGGCCCCAGCTGCCAGCAACGATGTCCGCCCGGGCGGTATGGGCCAGGCTGTGAACAGCTGT
GTGGGTGTCTCAACGGGGGCTCCTGTGATGCGGCCACGGGGGCCTGCCGCTGCCCCACTGGGTTCCTC
GGGACGGACTGCAACCTCACCTGTCCGCAGGGCCGCTTCGGCCCCAACTGCACCCACGTGTGTGGGTG
TGGGCAGGGGGCGGCCTGCGACCCTGTGACCGGCACCTGCCTCTGCCCCCCGGGGAGAGCCGGCGTCC
GCTGTGAGCGAGGCTGCCCCCAGAACCGGTTTGGCGTGGGCTGCGAGCACACCTGCTCCTGCAGAAAT
GGGGGCCTGTGCCACGCCAGCGGGGCCTGTGCCACGCCAGCAAGCGGCAGCTGCTCCTGTGGCCTGGG
CTGGACGGGGCGGCACTGCGAGCTGGCCTGTCCCCCTGGGCGCTACGGAGCCGCCTGCCATCTGGAGT
GCTCCTGCCACAACAACAGCACGTGTGAGCCTGCCACGGGCACCTGCCGCTGCGGCCCCGGCTTCTAT
GGCCAGGCCTGCGAGCACCCCTGTCCCCCTGGCTTCCACGGGGCTGGCTGCCAAAGGTTGTGCTGGTG
TCAACATGGAGCCCCCTGCGACCCCATCAGTGGCCGATGCCTCTGCCCTGCCGAATTCCACGAACACT
TCTGTGAGAGGGGGTGTGAGCCAGGTTCATTTGGAGAGGGCTGCCACCAGCGCTGTGACTGTGACGGG
GGGGCACCCTGTGACCCTGTCACCGGTCTCTGCCTTTGCCCACCAGGGCGCTCAGGAGCCACCTGTAA
CCTGGATTGCAGGGGGGGCCAGTTTGGGCCCAGCTGCACCCTGCACTGTGACTGCGGGGGTGGGGCTG
ACTGCGACCCTGTCAGTGGGCAGTGTCACTGTGTGGATGGCTACATGGGGCCCACGGGCCGGGGGGGT
GGGCCCCTCCGGCTCCCCGAGAACCCGTCCTTAGCCCAGGGCTCAGCGGGCACACTGCCCGCCTCCAG
CAGACCCACATCCCGGAGCGGTGGACCAGCGAGGCACTAGTAGAGGCAGTCCCGTGGAGCCCGCCTCT
CCAGTCCCAGCCAGAGGGGACTCTGGCCTTTGGTGACCACTGAGAAGGACACTTCACGGGCCCAGAGC
TCCTGGTACTGCCCTTCCTTTGAGGGCCGTGGAGGGCTGTGGACAGCCCAGCAACCTGTCGCTCTTGG
AGGCTGGTGTGGCCTTGAGGAGGGAAGCCTCGCATGGCCGCTGGAAGAGAGGCGCCTCCTGGCCTGGC
TCTGCAGAACCCAGGGGCACGCTCTGGGCCTGGGCTGAGGAAGTCCCGCTCTCCCCGCGGCTCTGAGT
TGGACTGAGGACAGGTGTGGGCGCCAGTGTGGGTGCAGTCACAGTGCAGGGTGCAGTCACAGTGCAGC
GTGC

| | | | |
|---|---|---|---|
| NOV37b, CG56449-09 | | | |
| Protein Sequence | SEQ ID NO: 860 | 1594 aa | MW at 166431.4kD |

MSFLEEARAAGRAVVLALVLLLLPAVPVGASVPPRPLLPLQPGMPHVCAEQELTLVGRRQPCVQALSH
TVPVWPAGCGWQAWCVGHERRTVYYMGYRQVYTTEARTVLRCCRGWTQQPDEEGCLSAECSASLCFHG
GRCVPGSAQPCHCPPGFQGPRCQYDVDECRTHNGGCQHRCVNTPGSYLCECKPGFRLHTDSRTCLAIN
SCALGNGGCQHHCVQLTITRHRCQCRPGFQLQEDGRHCVRRSPCANRNGSCMHRCQVVRGLARCECHV
GYQLAADGKACEDVDECAAGLAQCAHGCLNTQGSFKCVCHAGYELGADGRQCYRIEMEIVNSCEANNG
GCSHGCSHTSAGPLCTCPRGYELDTDQRTCIRCRRLCRQPVLQQVCTNNPGGYECGCYAGYRLSADGC
GCEDVDECASSRGGCEHHCTNLAGSFQCSCEAGYRLHEDRRGCSALEEPMVDLDGELPFVRPLPHIAV
LQDELPQLFQDDDVGADEEEAELRGEHTLTEKFVCLDDSFGHDCSLTCDDCRNGGTCLLGLDGCDCPE
GWTGLICNESCPPDTFGKNCSFSCSCQNGGTCDSVTGACRCPPGVSGTNCEDGCPKGYYGKHCRKKCN
CANRGRCHRLYGACLCDPGLYGRFCHLACPPWAFGPGCSEECQCVQPHTQSCDKRDGSCSCKAGFRGE
RCQAECEPGYFGPGCWQACTCPVGVACDSVSGECGKRCPAGFQGEDCGQECPVGTFGVNCSSSCSCGG
APCHGVTGQCRCPPGRTGEDCEAGECEGLWGLGCQEICPACHNAARCDPETGACLCLPGFVGSRCQDC
EAGWYGPSCQTMCSCANDGHCHQDTGHCSCAPGWTGFSCQRACDTGHWGPDCSHPCNCSAGHGSCDAI
SGLCLCEAGYVGPRCEQSECPQGHFGPGCEQRCQCQHGAACDHVSGACTCPAGWRGTFCEHACPAGFF
GLDCRSACNCTAGAACDAVTGSCLCPAGRRGPRCAESACPAHTYGHNCSQACACFNGASCDPVHGQCH
CAPGWMGPSCLQACPAGLYGDNCRHSCLCQNGGTCDPVSGHCACPEGWAGLACEVECLPRDVRAGCRH
SGGCLNGGLCDPHTGRCLCPAGWTGDKCQSPAACAKGTFGPHCEGRCACRWGGPCHLATGACLCPPGW
RGPHLSAACLRGWFGEACAQRCSCPPGAACHHVTGACRCPPGFTGSGCEQACPPGSFGEDCAQMCQCP
GENPACHPATGTCSCAAGYHGPSCQQRCPPGRYGPGCEQLCGCLNGGSCDAATGACRCPTGFLGTDCN
LTCPQGRFGPNCTHVCGCGQGAACDPVTGTCLCPPGRAGVRCERGCPQNRFGVGCEHTCSCRNGGLCH
ASGACATPASGSCSCGLGWTGRHCELACPPGRYGAACHLECSCHNNSTCEPATGTCRCGPGFYGQACE
HPCPPGFHGAGCQGLCWCQHGAPCDPISGRCLCPAGFHGHFCERGCEPGSFGEGCHQRCDCDGGAPCD
PVTGLCLCPPGRSGATCNLDCRRGQFGPSCTLHCDCGGGADCDPVSGQCHCVDGYMGPTCREGGPLRL
PENPSLAQGSAGTLPASSRPTSRSGGPARH

| | | |
|---|---|---|
| NOV37c, 191887507 | SEQ ID NO: 861 | 522 bp |
| DNA Sequence | ORF Start: at 2 | ORF Stop: at 521 |

GGATCCGTGCCTCGCTGGTCCACCGCTCATGGCTCCTGCCTCTGCCCCGCTGGCCGCCGGGGCCCCCG
CTGTGCCGAGACCTGCCCTGCCGGCCTGTACGGCGACAACTGTCGGCATTCCTGCCTCTGCCAGAACG
GAGGGACCTGTGACCCTGTCTCAGGCCTGCGAGCACCCCTGTCCCCCTGGCTTCCACGGGGCTGGCTG
CCAGGGGTTGTGCTGGTGTCAACATGGAGCCCCCTGCGACCCCATCAGTGGCCGATGCCTCTGCCCTG
CCGGCTTCCACGGCCACTTCTGTGAGAGGGATTGCAGAAGGGGCCAGTTTGGGCCCAGCTGCACCCTG
CACTGTGACTGCGGGGGTGGGGCTGACTGCGACCCTGTCAGTGGGCAGTGTCACTGTGTGGATGGCTA
CATGGGGCCCACGTGCCGGGAAGGTGGGCCCCTCCGGCTCCCCGAGAACCCGTCCTTAGCCCAGGGCT
CAGCGGGCACACTGCCCGCCTCCAGCAGACCCACATCCCGCTCGAG

| | | |
|---|---|---|
| NOV37c, 191887507 | | |
| Protein Sequence SEQ ID NO: 862 | 173 aa | MW at 17358.4kD |

DPCLAGPPLMAPASAPLAAGAPAVPRPALPACTATTVGIPASARTEGPVTLSQACEHPCPPGFHGAGC
QGLCWCQHGAPCDPISGRCLCPAGFHGHFCERDCRRGQFGPSCTLHCDCGGGADCDPVSGQCHCVDGY
MGPTCREGGPLRLPENPSLAQGSAGTLPASSRPTSRS

| | | |
|---|---|---|
| NOV37d, 316351371 | SEQ ID NO: 863 | 4255 bp |
| DNA Sequence | ORF Start: at 2 | ORF Stop: end of sequence |

CACCGGATCCACCATGTCGTTCCTTGAAGAGGCGAGGGCAGCGGGGCGCGCGGTGGTCCTGGCGTTGG
TGCTGCTGCTGCTCCCCGCCGTGCCCGTGGGCGCCAGCGTTCCGCCGCGGCCCCTGCTCCCGCTGCAG
CCCGGCATGCCCCACGTGTGTGCTGAGCAGGAGCTGACCCTGGTGGGCCGCCGCCAGCCGTGCGTGCA
GGCCTTAAGCCACACGGTGCCGGTGTGGAAGGCCGGCTGTGGGTGGCAGGCGTGGTGCGTGGGTCATG
AGCGGAGAACCGTCTACTACATGGGCTACAGGCAGGTGTATACCACGGAGGCCCGGACCGTGCTCAGG
TGCTGCCGAGGGTGGACGCAGCAGCCCGACGAGGAGGGCTGCCTCTCGGCTGAATGCAGCGCCGGCCT
CTGTTTTCACGGTGGCCGTTGTGTGCCAGGCTCAGCCCAGCCGTGTCACTGTCCCCCCGGCTTCCAGG
GACCCCGCTGTCAGTATGATGTGGACGAATGCCGAACCCACAACGGTGGCTGCCAGCACCGGTGCGTG
AACACCCCAGGCTCCTACCTCTGTGAGTGCAAGCCCGGCTTCCGGCTCCACACTGACAGCAGGACCTG
CCTGGCCATTAACTCCTGCGCCCTGGGCAATGGCGGCTGCCAGCACCACTGTGTCCAGCTCACAATCA
CTCGGCATCGCTGCCAGTGCCGGCCCGGGTTCCAGCTCCAGGAGGACGGCAGGCATTGTGTCCGTAGA
AGCCCGTGTGCCAACAGGAACGGCAGCTGCATGCACAGGTGCCAGGTGGTCCGGGGCCTCGCCCGCTG
TGAGTGCCACGTGGGCTATCAGCTAGCAGCGGACGGCAAGGCCTGTGAAGATGTGGACGAATGTGCCG
CAGGGCTGGCCCAGTGTGCCCATGGCTGCCTCAACACCCAGGGGTCCTTCAAGTGCGTGTGTCACGCG
GGCTATGAGCTGGGCGCCGATGGCCGGCAGTGCTACCGGATTGAGATGGAAATCGTGAACAGCTGTGA
GGCCAACAACGGCGGCTGCTCCCATGGCTGCAGCCACACCAGTGCTGGGCCCCTGTGCACCTGTCCCC
GCGGCTACGAGCTGGACACAGATCAGAGGACCTGCATCGATGTCGGCGACTGTGCAGACAGCCCGTGC
TGCCAGCAGGTGTGCACCAACAACCCTGGCGGGTACGAGTGCGGCTGCTACGCCGGCTACCGGCTCAG
TGCCGATGGCTGCGGCTGTGAGGATGTGGATGAGTGCGCCTCCAGCCGTGGCGGCTGCGAGCACCACT
GCACCAACCTGGCCGGCTCCTTCCAGTGCTCCTGCGAGGCCGGCTACCGGCTGCACGAGGACCGTAGG
GGCTGCAGCCCCCTGGAGGAGCCGATGGTGGACCTGGACGGCGAGCTGCCTTTCGTGCGGCCCCTGCC
CCACATTGCCGTGCTCCAGGACGAGCTGCCGCAACTCTTCCAGGATGACGACGTCGGGGCCGATGAGG
AAGAGGCAGAGTTGCGGGGCGAACACACGCTCACAGAGAAGTTTGTCTGCCTGGATGACTCCTTTGGC
CATGACTGCAGCTTGACCTGTGATGACTGCAGGAACGGAGGGACCTGTCTCCTGGGCCTGGATGGCTG
TGATTGCCCCGAGGGCTGGACTGGGCTCATCTGCAATGAGACTTGTCCTCCGGACACCTTTGGGAAGA
ACTGCAGCTTCTCCTGCAGCTGTCAGAATGGTGGGACCTGCGACTCTGTCACGGGGGCCTGCCGCTGC
CCCCCGGGTGTCAGTGGAACTAACTGTGAGGATGGCTGCCCCAAGGGCTACTATGGCAAGCACTGTCG
CAAGAAATGCAACTGTGCCAACCGGGGCCGGTGCCACCGCCTCTACGGGGCCTGCCTCTGCGACCCAG
GGCTCTACGGCCGCTTCTGCCACCTCACCTGCCCGCCGTGGGCCTTTGGGCCGGGCTGCTCGGAGGAG
TGCCAGTGTGTGCAGCCCCACACGCAGTCCTGTGACAAGAGGGATGGCAGCTGCTCCTGCAAGGCTGG
CTTCCGGGGCGAGCGCTGTCAGGCAGAGTGTGAGCCGGGCTACTTTGGGCCGGGGTGCTGGCAGGCAT
GCACCTGCCCAGTGGGCGTGGCCTGTGACTCCGTGAGCGGCGAGTGTGGGAAGCGGTGTCCTGCTGGC
TTCCAGGGAGAGGACTGTGGCCAAGAGTGCCCGGTGGGGACGTTTGGCGTGAACTGCTCGAGCTCCTG
CTCCTGTGGGGGGGCCCCCTGCCACGGGGTCACGGGGCAGTGCCGGTGTCCACCGGGGAGGACTGGGG
AAGACTGTGAGGCAGATTGTCCCGAGGGCCGCTGGGGGCTGGGCTGCCAGGAGATCTGCCCAGCATGC
CAGCACGCTGCCCGCTGCGACCCTGAGACCGGAGCCTGCCTGTGCCTCCCTGACTTCGTCGGCAGCCG
CTGCCAGGACGTGTGCCCAGCAGGCTGGTATGGTCCCAGCTGCCAGACAAGGTGCTCTTGTGCCAATG
ATGGGCACTGCCACCCAGCCACCGGACACTGCAGCTGTGCCCCCGGGTGGACCGGCTTTAGCTGCCAG
AGTGTCCCCAGGGCCACTTTGGGCCCGGCTGTGAGCAGCTGTGCCAGTGTCAGCATGGAGCAGCCTGT
GACCACGTCAGCGGGGCCTGCACCTGCCCGGCCGGCTGGAGGGGCACCTTCTGCGAGCATGCCTGCCC
GGCCGGCTTCTTTGGATTGGACTGTCGCAGTGCCTGCAACTGCACCGCCGGAGCTGCCTGTGATGCCG
TGAATGGCTCCTGCCTCTGCCCCGCTGGCCGCCGGGGCCCCCGCTGTGCCGAGAAGTGCCTCCCCCGG
CCGCTGCCTCTGCCCAGCCGGCTGGACTGGGGACAAGTGTCAGAGCCCCTGCCTGCGGGGCTGGTTTG
GAGAGGCCTGTGCCCAGCGCTGCAGCTGCCCGCCTGGCGCTGCCTGCCACCACGTCACTGGGGCCTGC
CGCTGTCCCCCTGGCTTCACTGGCTCCGGCTGCGAGCAGGGATGTCCGCCCGGGCGGTATGGGCCAGG
CTGTGAACAGCTGTGTGGGTGTCTCAACGGGGGCTCCTGTGATGCGGCCACGGGGGCCTGCCGCTGCC
CCACTGGGTTCCTCGGGACGGACTGCAACCTCACCTGTCCGCAGGGCCGCTTCGGCCCCAACTGCACC
GAGAGCCGGCGTCCGCTGTGAGCGAGGCTGCCCCCAGAACCGGTTTGGCGTGGGCTGCGAGCACACCT
GCTCCTGCAGAAATGGGGGCCTGTGCCACGCCAGCAACGGCAGCTGCTCCTGTGGCCTGGGCTGGACG
GGGCGGCACTGCGAGCTGGCCTGTCCCCCTGGGCGCTACGGAGCCGCCTGCCATCTGGAGTGCTCCTG
CCACAACAACAGCACGTGTGAGCCTGCCACGGGCACCTGCCGCTGCGGCCCCGGCTTCTATGGCCAGG
CCTGCGAGCACCCCTGTCCCCCTGGCTTCCACGGGGCTGGCTGCCAGGGGTTGTGCTGGTGTCAACAT
GGAGCCCCCTGCGACCCCATCAGTGGCCGATGCCTCTGCCCTGCCGGCTTCCACGGCCACTTCTGTGA
GAGGGGGTGTGAGCCAGGTTCATTTGGAGAGGGCTGCCACCAGCGCTGTGACTGTGACGGGGGGGCAC
CCTGTGACCCTGTCACCGGTCTCTGCCTTTGCCCACCAGGGCGCTCAGGAGCCACCTGTAACCTGGAT
TGCAGAAGGGGCCAGTTTGGGCCCAGCTGCACCCTGCACTGTGACTGCGGGGGTGGGGCTGACTGCGA
CCCTGTCAGTGGGCAGTGTCACTGTGTGGATGGCTACATGGGGCCCACGTGCCGGGAAGGTGGGCCCC
TCCGGCTCCCCGAGAACCCGTCCTTAGCCCAGGGCTCAGCGGGCACACTGCCCGCCTCCAGCAGACCC

| | | | |
|---|---|---|---|
| NOV37d, 316351371 | | | |
| Protein Sequence | SEQ ID NO: 864 | 1418 aa | MW at 148398.2kD |

TGSTMSFLEEARAAGRAVVLALVLLLLPAVPVGASVPPRPLLPLQPGMPHVCAEQELTLVGRRQPCVQ
ALSHTVPVWKAGCGWQAWCVGHERRTVYYMGYRQVYTTEARTVLRCCRGWTQQPDEEGCLSAECSAGL
CFHGGRCVPGSAQPCHCPPGFQGPRCQYDVDECRTHNGGCQHRCVNTPGSYLCECKPGFRLHTDSRTC
LAINSCALGNGGCQHHCVQLTITRHRCQCRPGFQLQEDGRHCVRRSPCANRNGSCMHRCQVVRGLARC
ECHVGYQLAADGKACEDVDECAAGLAQCAHGCLNTQGSFKCVCHAGYELGADGRQCYRIEMEIVNSCE
ANNGGCSHGCSHTSAGPLCTCPRGYELDTDQRTCIDVGDCADSPCCQQVCTNNPGGYECGCYAGYRLS
ADGCGCEDVDECASSRGGCEHHCTNLAGSFQCSCEAGYRLHEDRRGCSPLEEPMVDLDGELPFVRPLP
HIAVLQDELPQLFQDDDVGADEEEAELRGEHTLTEKFVCLDDSFGHDCSLTCDDCRNGGTCLLGLDGC
DCPEGWTGLICNETCPPDTFGKNCSFSCSCQNGGTCDSVTGACRCPPGVSGTNCEDGCPKGYYGKHCR
KKCNCANRGRCHRLYGACLCDPGLYGRFCHLTCPPWAFGPGCSEECQCVQPHTQSCDKRDGSCSCKAG
FRGERCQAECEPGYFGPGCWQACTCPVGVACDSVSGECGKRCPAGFQGEDCGQECPVGTFGVNCSSSC
SCGGAPCHGVTGQCRCPPGRTGEDCEADCPEGRWGLGCQEICPACQHAARCDPETGACLCLPDFVGSR
CQDVCPAGWYGPSCQTRCSCANDGHCHPATGHCSCAPGWTGFSCQRACDTGHWGPDCSHPCNCSAGHG
SCDAISGLCLCEAGYVGPRCEQQCPQGHFGPGCEQLCQCQHGAACDHVSGACTCPAGWRGTFCEHACP
AGFFGLDCRSACNCTAGAACDAVNGSCLCPAGRRGPRCAEKCLPRDVRAGCRHSGGCLNGGLCDPHTG
RCLCPAGWTGDKCQSPCLRGWFGEACAQRCSCPPGAACHHVTGACRCPPGFTGSGCEQGCPPGRYGPG
CEQLCGCLNGGSCDAATGACRCPTGFLGTDCNLTCPQGRFGPNCTHVCGCGQGAACDPVTGTCLCPPG
RAGVRCERGCPQNRFGVGCEHTCSCRNGGLCHASNGSCSCGLGWTGRHCETACPPGRYGAACHLECSC
HNNSTCEPATGTCRCGPGFYGQACEHPCPPGFHGAGCQGLCWCQHGAPCDPISGRCLCPAGFHGHFCE
RGCEPGSFGEGCHQRCDCDGGAPCDPVTGLCLCPPGRSGATCNLDCRPGQFGPSCTLHCDCGGGADCD
PVSGQCHCVDGYMGPTCREGGPLRLPENPSLAQGSAGTLPASSRPTSRSGGPARHGTG

| | | |
|---|---|---|
| NOV37e, 316935396 | SEQ ID NO: 865 | 5000 bp |
| DNA Sequence | ORF Start: at 28 | ORF Stop: TGA at 4987 |

TGCTGTTACAGGTGGAGGGCAGTGTAGTCTGAGCAGTACTCGTTGCTGCCGCGCGCGCCACCAGACAT
AATAGCTGACAGACTAACAGACTGTTCCTTTCCATGGGTCTTTTCTGCAGTCACCGTCCTTGACACGA
AGCTTTCTAGAAGATCTTCGCGAGGATCCACCATGTCGTTCCTTGAAGAGGCGAGGGCAGCGGGGCGC
GCGGTGGTCCTGGCGTTGGTGCTGCTGCTGCTCCCCGCCGTGCCCGTGGGCGCCAGCGTTCCGCCGCG
GCCCCTGCTCCCGCTGCAGCCCGGCATGCCCCACGTGTGTGCTGAGCAGGAGCTGACCCTGGTGGGCC
GCCGCCAGCCGTGCGTGCAGGCCTTAAGCCACACGGTGCCGGTGTGGAAGGCCGGCTGTGGGTGGCAG
GCGTGGTGCGTGGGTCATGAGCGGAGGACCGTCTACTACATGGGCTACAGGCAGGTGTATACCACGGA
GGCCCGGACCGTGCTCAGGTGCTGCCGAGGGTGGATGCAGCAGCCCGACGAGGAGGGCTGCCTCTCGG
ATGTGGGTGAGTGTGCCAACGCCAACGGGGGCTGTGCGGGTCGGTGCCGGGACACCGTGGGGGGCTTC
TACTGCCGCTGGCCCCCCCCCAGCCACCAGCTGCAGGGTGATGGCGAGACTTGCCAAGATGTGGACGA
ATGCCGAACCCACAACGGTGGCTGCCAGCACCGGTGCGTGAACACCCCAGGCTCCTACCTCTGTGAGT
GCAAGCCCGGCTTCCGGCTCCACACTGACAGCAGGACCTGCGCCATTAACTCCTGCGCCCTGGGCAAT
GGCGGCTGCCAGCACCACTGTGTCCAGCTCACAATCACTCGGCATCGCTGCCAGTGCCGGCCCGGGTT
CCAGCTCCAGGAGGACGGCAGGCATTGTGTCCGTAGAAGCCCGTGTGCCAACAGGAACGGCAGCTGCA
TGCACAGGTGCCAGGTGGTCCGGGGCCTCGCCCGCTGTGAGTGCCACGTGGGCTATCAGCTAGCAGCG
GACGGCAAGGCCTGTGAAGATGTGGACGAATGTGCCGCAGGGCTGGCCCAGTGTGCCCATGGCTGCCT
CAACACCCAGGGGTCCTTCAAGTGCGTGTGTCACGCGGGCTATGAGCTGGGCGCCGATGGCCGGCAGT
GCTACCGTATTGAGATGGAAATCGTGAACAGCTGTGAGGCCAACAACGGCGGCTGCTCCCATGGCTGC
AGCCACACCAGTGCTGGGCCCCTGTGCACCTGTCCCCGCGGCTACGAGCTGGACACAGATCAGAGGAC
CTGCATCAGATGTCGACGACTGTGCAGACAGCCCGTGCTGCAGCAGGTGTGCACCAACAACCCTGGCG
GGTACGAGTGCGGCTGCTACGCCGGCTACCGGCTCAGTGCCGATGGCTGCGGCTGCGAGGATGTGGAT
GAGTGCGCCTCCAGCCGTGGCGGCTGCGAGCACCACTGCACCAACCTGGCCGGCTCCTTCCAGTGCTC
CTGCGAGGCCGGCTACCGGCTGCACGAGGACCGTAGGGGCTGCAGCGCCCTGGAGGAGCCGATGGTGG
ACCTGGACGGCGAGCTGCCTTTCGTGCGGCCCCTGCCCCACATTGCCGTGCTCCAGGACGAGCTGCCG
CAACTCTTCCAGGATGACGACGTCGGGGCCGATGAGGAAGAGGCAGAGTTGCGGGGCGAACACACGCT
CACAGAGAAGTTTGTCTGCCTGGATGACTCCTTTGGCCATGACTGCAGCTTGACCTGTGATGACTGCA
GGAACGGAGGGACCTGCCTCCTGGGCCTGGATGGCTGTGATTGCCCCGAGGGCTGGACTGGGCTCATC
TGCAATGAGAGTTGTCCTCCGGACACCTTTGGGAAGAACTGCAGCTTCTCCTGCAGCTGTCAGAATGG
TGGGACCTGCGACTCTGTCACGGGGGCCTGCCGCTGCCCCCCGGGTGTCAGTGGAACTAACTGTGAGG
ATGGCTGCCCCAAGGGCTACTATGGCAAGCACTGTCGCAAGAAATGCAACTGTGCCAACCGGGGCCGG
TGCCACCGCCTCTACGGGGCCTGCCTCTGCGACCCAGGGCTCTACGGCCGCTTCTGCCACCTCGCCTG
CCCGCCGTGGGCCTTTGGGCCGGGCTGCTCGGAGGAGTGCCAGTGTGTGCAGCCCCACACGCAGTCCT
GTGACAAGAGGGATGGCAGCTGCTCCTGCAAGGCTGGCTTCCGGGGCGAGCGCTGTCAGGCAGAGTGT
GAGCCGGGCTACTTTGGGCCGGGGTGCTGGCAGGCATGCACCTGCCCAGTGGGCGTGGCCTGTGACTC
CGTGAGCGGCGAGTGTGGGAAGCGGTGTCCTGCTGGCTTCCAGGGAGAGGACTGTGGCCAAGAGTGCC
CGGTGGGGACCTTTGGCGTGAACTGCTCGAGCTCCTGCTCCTGTGGGGGGGCCCCCTGCCACGGGGTC
ACGGGGCAGTGCCGGTGTCCGCCGGGGAGGACTGGGGAAGACTGTGAGGCAGGTGAGTGTGAGGGCCT
CTGGGGGCTGGGCTGCCAGGAGATCTGCCCAGCATGCCATAACGCTGCTCGCTGCGACCCTGAGACCG
GAGCCTGCCTGTGCCTCCCTGGCTTTGTCGGCAGCCGCTGCCAGGACTGTGAGGCAGGCTGGTATGGT
CCCAGCTGCCAGACAATGTGCTCTTGTGCCAATGATGGGCACTGCCACCAAGACACGGGACACTGCAG
CTGTGCCCCCGGGTGGACCGGCTTTAGCTGCCAGAGAGCCTGTGATACTGGGCACTGGGGACCTGACT
GCAGCCACCCCTGCAACTGCAGCGCTGGCCACGGGAGCTGTGATGCCATCAGCGGCCTGTGTCTGTGT
GAGGCTGGCTACGTGGGCCCGCGGTGCGAGCAGTCAGAGTGTCCCCAGGGCCACTTTGGGCCCGGCTG
TGAGCAGCGGTGCCAGTGTCAGCATGGAGCAGCCTGTGACCACGTCAGCGGGGCCTGCACCTGCCCGG
CCGGCTGGAGGGGCACCTTCTGCGAGCATGCCTGCCCGGCCGGCTTCTTTGGATTGGACTGTCGCAGT
GCCTGCAACTGCACCGCCGGAGCTGCCTGTGATGCCGTGAATGGCTCCTGCCTCTGCCCCGCTGGCCG
CCGGGGCCCCCGCTGTGCCGAGAGTGCCTGCCCAGCCCACACCTACGGGCACAATTGCAGCCAGGCCT
GTGCCTGCTTTAACGGGGCCTCCTGTGACCCTGTCCACGGGCAGTGCCACTGTGCCCCTGGCTGGATG
GGGCCCTCCTGCCTGCAGGCCTGCCCTGCCGGCCTGTACGGCGACAACTGTCGGCATTCCTGCCTCTG
CCAGAACGGAGGGACCTGTGACCCTGTCTCAGGCCACTGTGCGTGCCCAGAGGGCTGGGCCGGCCTGG
CCTGTGAGGTAGAGTGCCTCCCCCGGGACGTCAGAGCTGGCTGCCGGCACAGCGGCGGTTGCCTCAAC
GGGGGCCTGTGTGACCCGCACACGGGCCGCTGCCTCTGCCCAGCCGGCTGGACTGGGGACAAGTGTCA
GAGCCCTGCAGCCTGTGCCAAGGGCACATTCGGGCCTCACTGTGAGGGGCGCTGTGCCTGCCGGTGGG
GAGGCCCCTGCCACCTTGCCACCGGGGCCTGCCTCTGCCCTCCGGGGTGGCGGGGGCCTCATCTTTCT
GCAGCCTGCCTGCGGGGCTGGTTTGGAGAGGCCTGTGCCCAGCGCTGCAGCTGCCCGCCTGGCGCTGC
CTGCCACCACGTCACTGGGGCCTGCCGCTGTCCCCCTGGCTTCACTGGCTCCGGCTGCGAGCAGGCCT
GCCCACCCGGCAGCTTTGGGGAGGACTGTGCGCAGATGTGCCAGTGTCCCGGTGAGAACCCGGCCTGC
CACCCTGCCACCGGGACCTGCTCATGTGCTGCTGGCTACCACGGCCCCAGCTGCCAGCAACGATGTCC
GCCCGGGCGGTATGGGCCAGGCTGTGAACAGCTGTGTGGGTGTCTCAACGGGGGCTCCTGTGATGCGG
CCACGGGGGCCTGCCGCTGCCCCACTGGGTTCCTCGGGACGGACTGCAACCTCACCTGTCCGCAGGGC
CGCTTCGGCCCCAACTGCACCCACGTGTGTGGGTGTGGGCAGGGGGCGGCCTGCGACCCTGTGACCGG
CACCTGCCTCTGCCCCCCGGGGAGAGCCGGCGTCCGCTGTGAGCGAGGCTGCCCCCAGAACCGGTTTG
GCGTGGGCTGCGAGCACACCTGCTCCTGCAGAAATGGGGGCCTGTGCCACGCCAGCAACGGCAGCTGC
TCCTGTGGCCTGGGCTGGACGGGGCGGCACTGCGAGCTGGCCTGTCCCCCTGGGCGCTACGGAGCCGC
CTGCCATCTGGAGTGCTCCTGCCACAACAACAGCACGTGTGAGCCTGCCACGGGCACCTGCCGCTGCG
GCCCCGGCTTCTATGGCCAGGCCTGCGAGCACCCCTGTCCCCCTGGCTTCCACGGGGCTGGCTGCCAG
GGGTTGTGCTGGTGTCAACATGGAGCCCCCTGCGACCCCATCAGTGGCCGATGCCTCTGCCCTGCCGG
CTTCCACGGCCACTTCTGTGAGAGGGGGTGTGAGCCAGGTTCATTTGGAGAGGGCTGCCACCAGCGCT
GTGACTGTGACGGGGGGGCACCCTGTGACCCTGTCACCGGTCTCTGCCTTTGCCCACCAGGGCGCTCA
GGAGCCACCTGTAACCTGGATTGCAGAAGGGGCCAGTTTGGGCCCAGCTGCACCCTGCACTGTGACTG
CGGGGGTGGGGCTGACTGCGACCCTGTCAGTGGGCAGTGTCACTGTGTGGATGGCTACATGGGGCCCA
CGTGCCGGGAAGCGGGCACACTGCCCGCCTCCAGCAGACCCACATCCCGGAGCGGTGGACCAGCGAGG
CACGTATTCCCCGGGCTCGAGGGTAAGCCTATCCCTAACCCTCTCCTCGGTCTCGATTCTACGCGTAC
CGGTCATCACCACCATCACCATTGAGTTTAATTCAT

| | | | |
|---|---|---|---|
| NOV37e, 316935396 | | | |
| Protein Sequence | SEQ ID NO: 866 | 1653 aa | MW at 173369.0kD |

SEQYSLLPRAPPDIIADRLTDCSFPWVFSAVTVLDTKLSRRSSRGSTMSFLEEARAAGRAVVLALVLL
LLPAVPVGASVPPRPLLPLQPGMPHVCAEQELTLVGRRQPCVQALSHTTPVWKAGCGWQAWCVGHERR
TVYYMGYRQVYTTEARTVLRCCRGWMQQPDEEGCLSDVGECANANGGCAGRCRDTVGGFYCRWPPPSH
QLQGDGETCQDVDECRTHNGGCQHRCVNTPGSYLCECKPGFRLHTDSRTCAINSCALGNGGCQHHCVQ
LTITRHRCQCRPGFQLQEDGRHCVRRSPCANRNGSCMHRCQVVRGLARCECHVGYQLAADGKACEDVD
ECAAGLAQCAHGCLNTQGSFKCVCHAGYELGADGRQCYRIEMEIVNSCEANNGGCSHGCSHTSAGPLC
TCPRGYELDTDQRTCIRCRRLCRQPVLQQVCTNNPGGYECGCYAGYRLSADGCGCEDVDECASSRGGC
EHHCTNLAGSFQCSCEAGYRLHEDRRGCSALEEPMVDLDGELPFVRPLPHIAVLQDELPQLFQDDDVG
ADEEEAELRGEHTLTEKFVCLDDSFGHDCSLTCDDCRNGGTCLLGLDGCDCPEGWTGLICNESCPPDT
FGKNCSFSCSCQNGGTCDSVTGACRCPPGVSGTNCEDGCPKGYYGKHCRKKCNCANRGRCHRLYGACL
CDPGLYGRFCHTACPPWAFGPGCSEECQCVQPHTQSCDKRDGSCSCKAGFRGERCQAECEPGYFGPGC
WQACTCPVGVACDSVSGECGKRCPAGFQGEDCGQECPVGTFGVNCSSSCSCGGAPCHGVTGQCRCPPG
RTGEDCEAGECEGLWGLGCQEICPACHNAARCDPETGACLCLPGFVGSRCQDCEAGWYGPSCQTMCSC
ANDGHCHQDTGHCSCAPGWTGFSCQRACDTGHWGPDCSHPCNCSAGHGSCDAISGLCLCEAGYVGPRC
EQSECPQGHFGPGCEQRCQCQHGAACDHVSGACTCPAGWRGTFCEHACPAGFFGLDCRSACNCTAGAA
CDAVNGSCLCPAGRRGPRCAESACPAETYGHNCSQACACFNGASCDPVHGQCHCAPGWMGPSCLQACP
AGLYGDNCRHSCLCQNGGTCDPVSGHCACPEGWAGLACEVECLPRDVRAGCRhSGGCLNGGLCDPHTG
RCLCPAGWTGDKCQSPAACAKGTFGPHCEGRCACRWGGPCHLATGACLCPPGWRGPHLSAACLRGWFG
EACAQRCSCPPGAACHHVTGACRCPPGFTGSGCEQACPPGSFGEDCAQMCQCPGENPACHPATGTCSC
AAGYHGPSCQQRCPPGRYGPGCEQLCGCLNGGSCDAATGACRCPTGFLGTDCNLTCPQGRFGPNCTHV
CGCGQGAACDPVTGTCLCPPGRAGVRCERGCPQNRFGVGCEHTCSCRNGGLCHASNGSCSCGLGWTGR
HCELACPPGRYGAACHLECSCHNNSTCEPATGTCRCGPGFYGQACEHPCPPGFHGAGCQGLCWCQHGA
PCDPISGRCLCPAGFHGHFCERGCEPGSFGEGCHQRCDCDGGAPCDPVTGLCLCPPGRSGATCNLDCR
RGQFGPSCTLHCDCGGGADCDPVSGQCHCVDGYMGPTCREAGTLPASSRPTSRSGGPARHVFPGLEGK
PIPNPLLGLDSTRTGHHHHHH

| | | |
|---|---|---|
| NOV37f, 317004318 | SEQ ID NO: 867 | 5005 bp |
| DNA Sequence | ORF Start: at 126 | ORF Stop: TGA at 4986 |

AACGGGTGGAGGGCAGTGTAGTCTGAGCAGTACTCGTTGCTGCCGCGCGCGCCACCAGACATAATAGC
TGACAGACTAACAGACTGTTCCTTTCCATGGGTCTTTTCTGCAGTCACCGTCCTTGACACGAAGCTCT
AGCCACCATGGAGACAGACACACTCCTGCTATGGGTACTGCTGCTCTGGGTTCCAGGTTCCACTGGTG
ACGCGGCCCAGCCGGCCAGGCGCGCGCGCCGTACGAAGCTTTCGCGAGGATCCAGCGTTCCGCCGCGG
CCCCTGCTCCCGCTGCAGCCCGGCATGCCCCACGTGTGTGCTGAGCAGGAGCTGACCCTGGTGGGCCG
CCGCCAGCCGTGCGTGCAGGCCTTAAGCCACACGGTGCCGGTGTGGAAGGCCGGCTGTGGGTGGCAGG
CGTGGTGCGTGGGTCATGAGCGGAGGACCGTCTACTACATGGGCTACAGGCAGGTGTATACCACGGAG
GCCCGGACCGTGCTCAGGTGCTGCCGAGGGTGGATGCAGCAGCCCGACGAGGAGGGCTGCCTCTCGGA
TGTGGGTGAGTGTGCCAACGCCAACGGGGGCTGTGCGGGTCGGTGCCGGGACACCGTGGGGGGCTTCT
ACTGCCGCTGGCCCCCCCCCAGCCACCAGCTGCAGGGTGATGGCGAGACTTGCCAAGATGTGGACGAA
TGCCGAACCCACAACGGTGGCTGCCAGCACCGGTGCGTGAACACCCCAGGCTCCTACCTCTGTGAGTG
CAAGCCCGGCTTCCGGCTCCACACTGACAGCAGGACCTGCGCCATTAACTCCTGCGCCCTGGGCAATG
GCGGCTGCCAGCACCACTGTGTCCAGCTCACAATCACTCGGCATCGCTGCCAGTGCCGGCCCGGGTTC
CAGCTCCAGGAGGACGGCAGGCATTGTGTCCGTAGAAGCCCGTGTGCCAACAGGAACGGCAGCTGCAT
GCACAGGTGCCAGGTGGTCCGGGGCCTCGCCCGCTGTGAGTGCCACGTGGGCTATCAGCTAGCAGCGG
ACGaCAAGGCCTGTGAAGATGTGGACGAATGTGCCGCAGGGCTGGCCCAGTGTGCCCATGGCTGCCTC
AACACCCAGGGGTCCTTCAAGTGCGTGTGTCACGCGGGCTATGAGCTGGGCGCCGATGGCCGGCAGTG
CTACCGTATTGAGATGGAAATCGTGAACAGCTGTGAGGCCAACAACGGCGGCTGCTCCCATGGCTGCA
GCCACACCAGTGCTGGGCCCCTGTGCACCTGTCCCCGCGGCTACGAGCTGGACACAGATCAGAGGACC
TGCATCAGATGTCGACGACTGTGCAGACAGCCCGTGCTGCAGCAGGTGTGCACCAACAACCCTGGCGG
GTACGAGTGCGGCTGCTACGCCGGCTACCGGCTCAGTGCCGATGGCTGCGGCTGCGAGGATGTGGATG
AGTGCGCCTCCAGCCGTGGCGGCTGCGAGCACCACTGCACCAACCTGGCCGGCTCCTTCCAGTGCTCC
TGCGAGGCCGGCTACCGGCTGCACGAGGACCGTAGGGGCTGCAGCGCCCTGGAGGAGCCGATGGTGGA
CCTGGACGGCGAGCTGCCTTTCGTGCGGCCCCTGCCCCACATTGCCGTGCTCCAGGACGAGCTGCCGC
AACTCTTCCAGGATGACGACGTCGGGGCCGATGAGGAAGAGGCAGAGTTGCGGGGCGAACACACGCTC
ACAGAGAAGTTTGTCTGCCTGGATGACTCCTTTGGCCATGACTGCAGCTTGACCTGTGATGACTGCAG
GAACGGAGGGACCTGCCTCCTGGGCCTGGATGGCTGTGATTGCCCCGAGGGCTGGACTGGGCTCATCT
GCAATGAGAGTTGTCCTCCGGACACCTTTGGGAAGAACTGCAGCTTCTCCTGCAGCTGTCAGAATGGT
GGGACCTGCGACTCTGTCACGGGGGCCTGCCGCTGCCCCCCGGGTGTCAGTGGAACTAACTGTGAGGA
TGGCTGCCCCAAGGGCTACTATGGCAAGCACTGTCGCAAGAAATGCAACTGTGCCAACCGGGGCCGGT
GCCACCGCCTCTACGGGGCCTGCCTCTGCGACCCAGGGCTCTACGGCCGCTTCTGCCACCTCGCCTGC
CCGCCGTGGGCCTTTGGGCCGGGCTGCTCGGAGGAGTGCCAGTGTGTGCAGCCCCACACGCAGTCCTG
TGACAAGAGGGATGGCAGCTGCTCCTGCAAGGCTGGCTTCCGGGGCGAGCGCTGTCAGGCAGAGTGTG
AGCCGGGCTACTTTGGGCCGGGGTGCTGGCAGGCATGCACCTGCCCAGTGGGCGTGGCCTGTGACTCC
GTGAGCGGCGAGTGTGGGAAGCGGTGTCCTGCTGGCTTCCAGGGAGAGGACTGTGGCCAAGAGTGCCC
GGTGGGGACCTTTGGCGTGAACTGCTCGAGCTCCTGCTCCTGTGGGGGGGCCCCCTGCCACGGGGTCA
CGGGGCAGTGCCGGTGTCCGCCGGGGAGGACTGGGGAAGACTGTGAGGCAGGTGAGTGTGAGGGCCTC
TGGGGGCTGGGCTGCCAGGAGATCTGCCCAGCATGCCATAACGCTGCTCGCTGCGACCCTGAGACCGG
AGCCTGCCTGTGCCTCCCTGGCTTTGTCGGCAGCCGCTGCCAGGACTGTGAGGCAGGCTGGTATGGTC
CCAGCTGCCAGACAATGTGCTCTTGTGCCAATGATGGGCACTGCCACCAAGACACGGGACACTGCAGC
TGTGCCCCCGGGTGGACCGGCTTTAGCTGCCAGAGAGCCTGTGATACTGGGCACTGGGGACCTGACTG
AGGCTGGCTACGTGGGCCCGCGGTGCGAGCAGTCAGAGTGTCCCCAGGGCCACTTTGGGCCCGGCTGT
GAGCAGCGGTGCCAGTGTCAGCATGGAGCAGCCTGTGACCACGTCAGCGGGGCCTGCACCTGCCCGGC
CGGCTGGAGGGGCACCTTCTGCGAGCATGCCTGCCCGGCCGGCTTCTTTGGATTGGACTGTCGCAGTG
CCTGCAACTGCACCGCCGGAGCTGCCTGTGATGCCGTGAATGGCTCCTGCCTCTGCCCCGCTGGCCGC
CGGGGCCCCCGCTGTGCCGAGAGTGCCTGCCCAGCCCACACCTACGGGCACAATTGCAGCCAGGCCTG
TGCCTGCTTTAACGGGGCCTCCTGTGACCCTGTCCACGGGCAGTGCCACTGTGCCCCTGGCTGGATGG
GGCCCTCCTGCCTGCAGGCCTGCCCTGCCGGCCTGTACGGCGACAACTGTCGGCATTCCTGCCTCTGC
GGGGCCTGTGTGACCCGCACACGGGCCGCTGCCTCTGCCCAGCCGGCTGGACTGGGGACAAGTGTCAG
AGCCCTGCAGCCTGTGCCAAGGGCACATTCGGGCCTCACTGTGAGGGGCGCTGTGCCTGCCGGTGGGG
AGGCCCCTGCCACCTTGCCACCGGGGCCTGCCTCTGCCCTCCGGGGTGGCGGGGGCCTCATCTTTCTG
CAGCCTGCCTGCGGGGCTGGTTTGGAGAGGCCTGTGCCCAGCGCTGCAGCTGCCCGCCTGGCGCTGCC
TGCCACCACGTCACTGGGGCCTGCCGCTGTCCCCCTGGCTTCACTGGCTCCGGCTGCGAGCAGGCCTG
CCCACCCGGCAGCTTTGGGGAGGACTGTGCGCAGATGTGCCAGTGTCCCGGTGAGAACCCGGCCTGCC
ACCCTGCCACCGGGACCTGCTCATGTGCTGCTGGCTACCACGGCCCCAGCTGCCAGCAACGATGTCCG
CCCGGGCGGTATGGGCCAGGCTGTGAACAGCTGTGTGGGTGTCTCAACGGGGGCTCCTGTGATGCGGC
CACGGGGGCCTGCCGCTGCCCCACTGGGTTCCTCGGGACGGACTGCAACCTCACCTGTCCGCAGGGCC
GCTTCGGCCCCAACTGCACCCACGTGTGTGGGTGTGGGCAGGGGGCGGCCTGCGACCCTGTGACCGGC
ACCTGCCTCTGCCCCCCGGGGAGAGCCGGCGTCCGCTGTGAGCGAGGCTGCCCCCAGAACCGGTTTGG
CGTGGGCTGCGAGCACACCTGCTCCTGCAGAAATGGGGGCCTGTGCCACGCCAGCAACGGCAGCTGCT
CCTGTGGCCTGGGCTGGACGGGGCGGCACTGCGAGCTGGCCTGTCCCCCTGGGCGCTACGGAGCCGCC
TGCCATCTGGAGTGCTCCTGCCACAACAACAGCACGTGTGAGCCTGCCACGGGCACCTGCCGCTGCGG
CCCCGGCTTCTATGGCCAGGCCTGCGAGCACCCCTGTCCCCCTGGCTTCCACGGGGCTGGCTGCCAGG
GGTTGTGCTGGTGTCAACATGGAGCCCCCTGCGACCCCATCAGTGGCCGATGCCTCTGCCCTGCCGGC
TTCCACGGCCACTTCTGTGAGAGGGGGTGTGAGCCAGGTTCATTTGGAGAGGGCTGCCACCAGCGCTG
TGACTGTGACGGGGGGGCACCCTGTGACCCTGTCACCGGTCTCTGCCTTTGCCCACCAGGGCGCTCAG
GAGCCACCTGTAACCTGGATTGCAGAAGGGGCCAGTTTGGGCCCAGCTGCACCCTGCACTGTGACTGC
GGGGGTGGGGCTGACTGCGACCCTGTCAGTGGGCAGTGTCACTGTGTGGATGGCTACATGGGGCCCAC
GTGCCGGGAAGCGGGCACACTGCCCGCCTCCAGCAGACCCACATCCCGGAGCGGTGGACCAGCGAGGC
ACGAATTCCCCGGGCTCGAGGGTAAGCCTATCCCTAACCCTCTCCTCGGTCTCGATTCTACGCGTACC
GGTCATCACCACCATCACCATTGAGTTTAATTCATTGATTT

| | | | |
|---|---|---|---|
| NOV37f, 317004318 | | | |
| Protein Sequence | SEQ ID NO: 868 | 1620 aa | MW at 169975.0kD |

HEALATMETDTLLLWVLLLWVPGSTGDAAQPARRARRTKLSRGSSVPPRPLLPLQPGMPHVCAEQELT
LVGRRQPCVQALSHTVPVWKAGCGWQAWCVGHERRTVYYMGYRQVYTTEARTVLRCCRGWMQQPDEEG
CLSDVGECANANGGCAGRCRDTVGGFYCRWPPPSHQLQGDGETCQDVDECRTHNGGCQHRCVNTPGSY
LCECKPGFRLHTDSRTCAINSCALGNGGCQHHCVQLTITRHRCQCRPGFQLQEDGRHCVRRSPCANRN
GSCMHRCQVVRGLARCECHVGYQLAADGKACEDVDECAAGLAQCAHGCLNTQGSFKCVCHAGYELGAD
GRQCYRIEMEIVNSCEANNGGCSHGCSHTSAGPLCTCPRGYELDTDQRTCIRCRRLCRQPVLQQVCTN
NPGGYECGCYAGYRLSADGCGCEDVDECASSRGGCEHHCTNLAGSFQCSCEAGYRLHEDRRGCSALEE
PMVDLDGELPFVRPLPHIAVLQDELPQLFQDDDVGADEEEAELRGEHTLTEKFVCLDDSFGHDCSLTC
DDCRNGGTCLLGLDGCDCPEGWTGLICNESCPPDTFGKNCSFSCSCQNGGTCDSVTGACRCPPGVSGT
NCEDGCPKGYYGKHCRKKCNCANRGRCHRLYGACLCDPGLYGRFCHLACPPWAFGPGCSEECQCVQPH
TQSCDKRDGSCSCKAGFRGERCQAECEPGYFGPGCWQACTCPVGVACDSVSGECGKRCPAGFQGEDCG
QECPVGTFGVNCSSSCSCGGAPCHGVTGQCRCPPGRTGEDCEAGECEGLWGLGCQEICPACHNAARCD
PETGACLCLPGFVGSRCQDCEAGWYGPSCQTMCSCANDGHCHQDTGHCSCAPGWTGFSCQRACDTGHW
GPDCSHPCNCSAGHGSCDAISGLCLCEAGYVGPRCEQSECPQGHFGPGCEQRCQCQHGAACDHVSGAC
TCPAGWRGTFCEHACPAGFFGLDCRSACNCTAGAACDAVNGSCLCPAGRRGPRCAESACPAHTYGHNC
SQACACFNGASCDPVHGQCHCAPGWMGPSCLQACPAGLYGDNCRHSCLCQNGGTCDPVSGHCACPEGW
AGLACEVECLPRDVRAGCRHSGGCLNGGLCDPHTGRCLCPAGWTGDKCQSPAACAKGTFGPHCEGRCA
CRWGGPCHLATGACLCPPGWRGPHLSAACLRGWFGEACAQRCSCPPGAACHHVTGACRCPPGFTGSGC
EQACPPGSFGEDCAQMCQCPGENPACHPATGTCSCAAGYHGPSCQQRCPPGRYGPGCEQLCGCLNGGS
CDAATGACRCPTGFLGTDCNLTCPQGRFGPNCTHVCGCGQGAACDPVTGTCLCPPGRAGVRCERGCPQ
NRFGVGCEHTCSCRNGGLCHASNGSCSCGLGWTGRHCELACPPGRYGAACHLECSCHNNSTCEPATGT
CRCGPGFYGQACEHPCPPGFHGAGCQGLCWCQHGAPCDPISGRCLCPAGFHGHFCERGCEPGSFGEGC
HQRCDCDGGAPCDPVTGLCLCPPGRSGATCNLDCRRGQFGPSCTLHCDCGGGADCDPVSGQCHCVDGY
MGPTCREAGTLPASSRPTSRSGGPARHEFPGLEGKPIPNPLLGLDSTRTGHHHHHH

| | | |
|---|---|---|
| NOV37g, CG56449-01 | SEQ ID NO: 869 | 7337 bp |
| DNA Sequence | ORF Start: ATG at 1 | ORF Stop: TGA at 4213 |

ATGCCCATGGGACATTCTGACAGGTGGTCTTGGCGTCTCCTGAGGCTGGCACTGCCACTCCCAGTCTG
GTTGCCGGCTGGGGGTGGCCGAGGCGCTGACTCTCCATGTCTCTGTTCCAGGCCCCACGTGTGTGCTG
AGCAGGAGCTGACCCTGGTGGGCCGCCGCCAGCCGTGCGTGCAGGCCTTAAGCCACACGGTGCCGGTG
TGGAAGGCCGGCTGTGGGTGGCAGGCGTGGTGCGTGGGTCATGAGCGGAGGACCGTCTACTACATGGG
CTACAGGCAGGTGTATACCACGGAGGCCCGGACCGTGCTCAGGTGCTGCCGAGGGTGGATGCAGCAGC
CCGACGAGGAGGGCTGCCTCTCGGATGTGGGTGAGTGTGCCAACGCCAACGGGGGCTGTGCGGGTCGG
TGCCGGGACACCGTGGGGGGCTTCTACTGCCGCTGGCCCCCCCCCAGCCACCAGCTGCAGGGTGATGG
CGAGACTTGCCAAGATGTGGACGAATGCCGAACCCACAACGGTGGCTGCCAGCACCGGTGCGTGAACA
CCCCAGGCTCCTACCTCTGTGAGTGCAAGCCCGGCTTCCGGCTCCACACTGACAGCAGGACCTGCGCC
ATTAACTCCTGCGCCCTGGGCAATGGCGGCTGCCAGCACCACTGTGTCCAGCTCACAATCACTCGGCA
TCGCTGCCAGTGCCGGCCCGGGTTCCAGCTCCAGGAGGACGGCAGGCATTGTGTCCGTAGAAGCCCGT
GTGCCAACAGGAACGGCAGCTGCATGCACAGGTGCCAGGTGGTCCGGGGCCTCGCCCGCTGTGAGTGC
CACGTGGGCTATCAGCTAGCAGCGGACGGCAAGGCCTGTGAAGATGTGGACGAATGTGCCGCAGGGCT
GGCCCAGTGTGCCCATGGCTGCCTCAACACCCAGGGGTCCTTCAAGTGCGTGTGTCACGCGGGCTATG
AGCTGGGCGCCGATGGCCGGCAGTGCTACCGTATTGAGATGGAAATCGTGAACAGCTGTGAGGCCAAC
AACGGCGGCTGCTCCCATGGCTGCAGCCACACCAGTGCTGGGCCCCTGTGCACCTGTCCCCGCGGCTA
CGAGCTGGACACAGATCAGAGGACCTGCATCAGATGTCGACGACTGTGCAGACAGCCCGTGCTGCAGC
AGGTGTGCACCAACAACCCTGGCGGGTACGAGTGCGGCTGCTACGCCGGCTACCGGCTCAGTGCCGAT
GGCTGCGGCTGCGAGGATGTGGATGAGTGCGCCTCCAGCCGTGGCGGCTGCGAGCACCACTGCACCAA
CCTGGCCGGCTCCTTCCAGTGCTCCTGCGAGGCCGGCTACCGGCTGCACGAGGACCGTAGGGGCTGCA
GCGCCCTGGAGGAGCCGATGGTGGACCTGGACGGCGAGCTGCCTTTCGTGCGGCCCCTGCCCCACATT
GCCGTGCTCCAGGACGAGCTGCCGCAACTCTTCCAGGATGACGACGTCGGGGCCGATGAGGAAGAGGC
AGAGTTGCGGGGCGAACACACGCTCACAGAGAAGTTTGTCTGCCTGGATGACTCCTTTGGCCATGACT
GCAGCTTGACCTGTGATGACTGCAGGAACGGAGGGACCTGCCTCCTGGGCCTGGATGGCTGTGATTGC
CCCGAGGGCTGGACTGGGCTCATCTGCAATGAGAGTTGTCCTCCGGACACCTTTGGGAAGAACTGCAG
CTTCTCCTAAAGCTGTCAGAATGGTGGGACCTGCGACTCTGTCACGGGGGCCTGCCGCTGCCCCCCGG
GTGTCAGTGGAACTAACTGTGAGGATGGCTGCCCCAAGGGCTACTATGGCAAGCACTGTCGCAAGAAA
TGCAACTGTGCCAACCGGGGCCGGTGCCACCGCCTCTACGGGGCCTGCCTCTGCGACCCAGGGCTCTA
CGAACGCTTCTGCCACCTCGCCTGCCCGCCGTGGGCCTTTGGGCCGGGCTGCTCGGAGGAGTGCCAGT
GTGTGCAGCCCCACACGCAGTCCTGTGACAAGAGGGATGGCAGCTGCTCCTGCAAGGCTAACTTCCGG
GGCGAGCGCTGTCAGGCAGAGTGTGAGCCGGGCTACTTTGGGCCGGGGTGCTGGCAGGCATGCACCTG
CCCAGTGGGCGTGGCCTGTGACTCCGTGAGCGGCGAGTGTGGGAAGCGGTGTCCTGCTGGCTTCCAGG
GAGAGGACTGTGGCCAAGAGTGCCCGGTGGGGACCTTTGGCGTGAACTGCTCGAGCTCCTGCTCCTGT
GGGGGGGCCCCCTGCCACGGGGTCACGGGGCAGTGCCGGTGTCCGCCGGGGAGGACTGGGAAAGACTG
TGAGGCAGGTGAGTGTGAGGGCCTCTGGGGGCTGGGCTGCCAGGAGATCTGCCCAGCATGCCATAACG
CTGCTCGCTGCGACCCTGAGACCGGAGCCTGCCTGTGCCTCCCTGGCTTTGTCGGCAGCCGCTGCCAG
GACTGTGAGGCAGGCTGGTATGGTCCCAGCTGCCAGACAATGTGCTCTTGTGCCGCTGATGGGCACTG
CCACCAAGACACGGGACACTGCAGCTGTGCCCCCGGGTGGACCGGCTTTAGCTGCCAGAGAGCCTGTG
ATACTGGGCACTGGGGACCTGACTGCAGCCACCCCTGCAACTGCAGCGCTGGCCACGGGAGCTGTGAT
GCCATCAGCGGCCTGTGTCTGTGTGAGGCTGGCTACGTGGGCCCGCGGTGCGAGCAGTCAGAGTGTCC
CCAGGGCCACTTTGGGCCCGGCTGTGAGCAGCGGTGCCAGTGTCAGCATGGAGCAGCCTGTGACCACG
TCAGCGGGGCCTGCACCTGCCCGGCCGGCTGGAGGGGCACCTTCTGCGAGCATGCCTGCCCGGCCGGC
TTCTTTGGATTGGACTGTCGCAGTGCCTGCAACTGCACCGCCGGAGCTGCCTGTGATGCCGTGAATGG
CTCCTGCCTCTGCCCCGCTGGCCGCCGGGGCCCCCGCTGTGCCGAGAGTGCCTGCCCAGCCCACACCT
ACGGGCACAATTGCAGCCAGGCCTGTGCCTGCTTTAACGGGGCCTCCTGTGACCCTGTCCACGGGCAG
TGCCACTGTGCCCCTGGCTGGATGGGGCCCTCCTGCCTGCAGGCCTGCCCTGCCGGCCTGTACGGCGA
CAACTGTCGGCATTCCTGCCTCTGCCAGAACGGAGGGACCTGTGACCCTGTCTCAGGCCACTGTGCGT
GCCCAGAGGGCTGGGCCGGCCTGGCCTGTGAGGTAGAGTGCCTCCCCCGGGACGTCAGAGCTGGCTGC
CGGCACAGCGGCGGTTGCCTCAACGGGGGCCTGTGTGACCCGCACACGGGCCGCTGCCTCTGCCCAGC
CGGCTGGACTGGGGACAAGTGTCAGAGCCCTGCAGCCTGTGCCAAGGGCACATTCGGGCCTCACTGTG
AGGGGCGCTGTGCCTGCCGGTGGGGAGGCCCCTGCCACCTTGCCACCGGGGCCTGCCTCTGCCCTCCG
GGGTGGCGGGGGCCTCATCTTTCTGCAGCCTGCCTGCGGGGCTGGTTTGGAGAGGCCTGTGCCCAGCG
CTGCAGCTGCCCGCCTGGCGCTGCCTGCCACCACGTCACTGGGGCCTGCCGCTGTCCCCCTGGCTTCA
CTGGCTCCGGCTGCGAGCAGGCCTGCCCACCCGGCAGCTTTGGGGAGGACTGTGCGCAGATGTGCCAG
TGTCCCGGTGAGAACCCGGCCTGCCACCCTGCCACCGGGACCTGCTCATGTGCTGCTGGCTACCACGG
CCCCAGCTGCCAGCAACGATGTCCGCCCGGGCGGTATGGGCCAGGCTGTGAACAGCTGTGTGGGTGTC
TCAACGGGGGCTCCTGTGATGCGGCCACGGGGGCCTGCCGCTGCCCCACTGGGTTCCTCGGGACGGAC
TGCAACCTCACCTGTCCGCAGGGCCGCTTCGGCCCCAACTGCACCCACGTGTGTGGGTGTGGGCAAAG
GGCGGCCTGCGACCCTGTGACCGGCACCTGCCTCTGCCCCCCGGGGAGAGCCGGCGTCCGCTGTGAGC
GAGGCTGCCCCCAGAACCGGTTTGGCGTGGGCTGCGAGCACACCTGCTCCTGCAGAAATGGGGGCCTG
TGCCACGCCAGCAAGCGGCAGCTGCTCCTGTGGCCTGGGCTGGACGGGGCGGCACTGCGAGCTGGCCT
GTCCCCCTGGGCGCTACGGAGCCGCCTGCCATCTGGAGTGCTCCTGCCACAACAACAGCACGTGTGAG
CCTGCCACGGGCACCTGCCGCTGCGGCCCCGGCTTCTATGGCCAGGCCTGCGAGCACCCCTGTCCCCC
TGGCTTCCACGGGGCTGGCTGCCAGGGGTTGTGCTGGTGTCAACATGGAGCCCCCTGCGACCCCATCA
GTGGCCGATGCCTCTGCCCTGCCGGCTTCCACGGCCACTTCTGTGAGAGGGGGTGTGAGCCAGGTTCA
CTGCCTTTGCCCACCAGGGCGCTCAGGAGCCACCTGTAACCTGGATTGCAGAAGGGGCCAGTTTGGGC
CCAGCTGCACCCTGCACTGTGACTGCGGGGGTGGGGCTGACTGCGACCCTGTCAGTGGGCAGTGTCAC
TGTGTGGATGGCTACATGGGGCCCACGTGCCGGGAAGGTGGGCCCCTCCGGCTCCCCGAGAACCCGTC
CTTAGCCCAGGGCTCAGCGGGCACACTGCCCGCCTCCAGCAGACCCACATCCCGGAGCGGTGGACCAG
CGAGGCACTAGTAGAGGCAGTCCCGTGGAGCCCGCCTCTCCAGTCCCAGCCAGAGGGGACCCTGGCCT
TTGGTGACCACTGAGAAGGACACTTCACGGGCCCAGAGCTCCTGGTACTGCCCTTCCTTTGAGGGCCG
AGGAGGGCTGTGGACAGCCCAGCAACCTGTCGCTCTTGGAGGCTGGTGTGGCCTTGAGGAGGGAAGAA
TCGCATGGCCGCTGGAAGAGAGGCGCCTCCTGGCCTGGCTCTGCAGAACCCAGGGGCACGCTCTGGGC
CTGGGCTGAGGAAGTCCCGCTCTCCCCGCGGCTCTGAGTTGGACTGAGGACAGGTGTGGGCGCCAGTG
TGGGTGCAGGCGCAGGTGCAGGCACAGGGCCACTGTCCTCCAGGCAGGCTTTTTGGTGCTAGGCCCTG
GGACTGGAAGTCGCCCAGCCCGTATTTATGTAAAGGTATTTATGGGCCACTGCACATGCCCGCTGCAG
CCCTGGGATCAGCTGGAAGCTGCCTGTCATCTCCTGCCCAATCCCCAGAAACCCTGATTCAGGTCTGC
AGGCTCCTGCGGGCTCACCAGGCTGCTGGCTCCGGTACCATGTACCTAAAGGTAAAAAAAGGAGCAGG
CAACCTCCTCGTGGCCTGTGTGTTTGCTGTGTTACGTGGACTCTGTGTGGGCTCCTCCCTGGGGCCCG
GCCAGCATAACGGTGCACCCAGGGACCTCCCAGTGCACCCGGGGCCCTTTGCAGGGGTGGGGGTGCCA
CACAAGTGAAGAAGTTGGGACTCATCTCAGTTCCCAGTGCTATTGAGGAGAACGCTGGGGCTGCATTC
ATTACCGCTGAGACCCAGAGACTGGCTGTTCCCAGAGAATGGCCCAGGGGGAGGAGAACTGGTGTAAA
GGGGCAACCTGGACTGAGGCCGAACTCCCTTGGGCTCACCCCACCCACCCCTACCTGAGCATCAGCAG
TGGGGGGAGGGCAGCATCGCAGGGGCAGGGACTCCCTGGGTGAGGACAGACCAGCCCTCCCGAGCACC
TGGCACTCATGGGCTGAGGCTGACTTCTCCTGGAAGAAGGGCCCAGAGTGGAAGGAAGAGGCAGAGGC
TAGAGGTGGTGGCTGGGGGCTCCTCTGCAGAGTGGGGTGGCCAATGGAGAGGGCTGCACTCACACCGC
ACATAGGAACTCTCTCTCCCTTAAGAAGGCCCCCTTAGGGTCTGGGCTGCCGCCCCCATCACCCTAAA
CCAGCCAAAGGTAGCTGAGGCCCCAGGGCAGACAATTTCACCAGCAGGANGAGGAGGAGTCCAGTGAG
CTTGGTTGCTCACAGACAGCAAGGGAGCTGTCACAGAGGAAGCTGATGAATGGACCGCTGTGGGGAGA
CTTTAAAGTAGAACAGTGATAAGGGAGGGCAGGATGGTGGGGATGCAGAAGCAGCAGCCAGAGAGAGA
CGGACTGGGGTGCAGACGGAGTGTGGAAAACGCATACCTTGAAATGAAGCATCCAGCAGATGGGGTGA
GTGGATACAGCTCAGGAGATTCTCCCAGGAATAGCAGGGAGGCGTAAAGAGAGACAACGTACAGAGAT
AGATGAATGGAAATGGGTAAGGGAGGTGTTCATTCACATCCATCTAACTGCAAAATACAAAAGTAAGA
AGTCATTGACATGAAGCAACGACGACCAAGACGTTCTCAGATCTAAAGGTGAATGATCTCAGTCAGCC
TGGAAATGCACAAGGTGGAAAAATAACATAAAAAAGCCATAAGACCTTGAAGAACATCAATGTCAAAG
ATAAATTCTAAAGTCCCAGAGAAAAAAGAATGGGAATCAAATTGACCTCAGACTATACGTGAGAAACA
CGGAGAGCCAGAAAACTGTGATGTTCCATCCTCAGAGTTTGAAGGAAATATTTGAAGGCTGAATTTTA
CATCCAGCTAAACTATCAAAGGCATGCAAAGTCCATGTTATTCTTAGGCCTTCAAGGCCTCGGCCATT
TTTCTACAGAAAAGCCTGATTTTAAAATGCTCTTAGAGACGTTCTCCAGCCAGAAGAGAAAGAAGCCA
GGAGGGTGCTCTGAGATATTCAGTCACCACAGTTCCCAAATGGCCTAGGAATTCAGAGAGTCAGAATA
TCACCATTACTCCCCAATGGGAACCCCCGACAGTCTCAGCATGGTGTGAGGGTGTGGACGGGGGGCCT
GGCAGGTACCAATCACTCATCCCGCTCAGTGAAGACACAGTGTTCAGCTACGGAAGCCATAAGGCAGG
CCGAGCTTCTGCCCATCCGGAGGAAATCTCAGCTATCCAACGGCGGTCAGGAGCAGAGGAAAATAAAG
CAGAATAACTAGAAAACACGCTCACAGATCCTAATGTTAACGGTTACAAATGACGACGGAAAAACAAA
CTCCTGACCATATATTATATAGTTTCAAGCAGCAAGAAGGAGGATATTGAACATTCTCAACACACATA
ATAAACGCTTGAGATGATGATATGCTCATTACCCTGATTTGATCACTAGACATACCATGTATCAAAAC
ATCACTGTGTATCCGATGAATATCTACAATTATTGTCAATTAAAACATCATTAAAAAACAA

| | | | |
|---|---|---|---|
| NOV37g, CG56449-01 | | | |
| Protein Sequence | SEQ ID NO: 870 | 1404 aa | MW at 147886.8kD |

MPMGHSDRWSWRLLRLALPLPVWLPAGGGRGADSPCLCSRPHVCAEQELTLVGRRQPCVQALSHTVPV
WKAGCGWQAWCVGHERRTVYYMGYRQVYTTEARTVLRCCRGWMQQPDEEGCLSDVGECANANGGCAGR
CRDTVGGFYCRWPPPSHQLQGDGETCQDVDECRTHNGGCQHRCVNTPGSYLCECKPGFRLHTDSRTCA
INSCALGNGGCQHHCVQLTITRHRCQCRPGFQLQEDGRHCVRRSPCANRNGSCMHRCQVVRGLARCEC
HVGYQLAADGKACEDVDECAAGLAQCAHGCLNTQGSFKCVCHAGYELGADGRQCYRIEMEIVNSCEAN
NGGCSHGCSHTSAGPLCTCPRGYELDTDQRTCIRCRRLCRQPVLQQVCTNTPGGYECGCYAGYRLSAD
GCGCEDVDECASSRGGCEHHCTNLAGSFQCSCEAGYRLHEDRRGCSALEEPMVDLDGELPFVRPLPHI
AVLQDELPQLFQDDDVGADEEEAELRGEHTLTEKFVCLDDSFGHDCSLTCDDCRNGGTCLLGLDGCDC
PEGWTGLICNESCPPDTFGKNCSFSCSCQNGGTCDSVTGACRCPPGVSGTNCEDGCPKGYYGKHCRKK
CNCANRGRCHRLYGACLCDPGLYGRFCHLACPPWAFGPGCSEECQCVQPHTQSCDKRDGSCSCKAGFR
GERCQAECEPGYFGPGCWQACTCPVGVACDSVSGECGKRCPAGFQGEDCGQECPVGTFGVNCSSSCSC
GGAPCHGVTGQCRCPPGRTGEDCEAGECEGLWGLGCQEICPACHNAARCDPETGACLCLPGFVGSRCQ
DCEAGWYGPSCQTMCSCANDGHCHQDTGHCSCAPGWTGFSCQPACDTGHWGPDCSHPCNCSAGHGSCD
ASGLCLCEAGYVTGPRCEQSECPQGHFGPGCEQRCQCQHGAACDHVSGACTCPAGWRGTFCEHACPAG
FFGLDCRSACNCTAGAACDAVNGSCLCPAGRRGPRCAESACPAHTYGHNCSQACACFNGASCDPVHGQ
CHCAPGWMGPSCLQACPAGLYGDNCRHSCLCQNGGTCDPVSGHCACPEGWAGLACEVECLPRDVRAGC
RHSGGCLNGGLCDPHTGRCLCPAGWTGDKCQSPAACAKGTFGPHCEGRCACRWGGPCHLATGACLCPP
GWRGPHLSAACLRGWFGEACAQRCSCPPGAACHHVTGACRCPPGFTGSGCEQACPPGSFGEDCAQMCQ
CPGENPACHPATGTCSCAAGYHGPSCQQRCPPGRYGPGCEQLCGCLNGGSCDAATGACRCPTGFLGTD
CNLTCPQGRFGPNCTHVCGCGQGAACDPVTGTCLCPPGRAGVRCERGCPQNRFGVGCEHTCSCRNGGL
CHASKRQLLLWPGLDGAALRAGLSPWALRSRLPSGVLLPQQQHV

| | | |
|---|---|---|
| NOV37h, CG56449-02 | SEQ ID NO: 871 | 7319 bp |
| DNA Sequence | ORF Start: ATG at 1 | ORF Stop: TGA at 4195 |

ATGCCCATGGGACATTCTGACAGGTGGTCTTGGCGTCTCCTGAGGCTGGCACTGCCACTCCCAGTCTG
GTTGCCGGCTGGGGGTGGCCGAGGCGCTGACTCTCCATGTCTCTGTTCCAGGCCCCACGTGTGTGCTG
AGCAGGAGCTGACCCTGGTGGGCCGCCGCCAGCCGTGCGTGCAGGCCTTAAGCCACACGGTGCCGGTG
TGGAAGGCCGGCTGTGGGTGGCAGGCGTGGTGCGTGGGTCATGAGCGGAGAACCGTCTACTACATGGG
CTACAGGCAGGTGTATACCACGGAGGCCCGGACCGTGCTCAGGTGCTGCCGAGGGTGGACGCAGCAGC
CCGACGAGGAGGGCTGCCTCTCGGCTGAATGCAGCGCCAGCCTCTGTTTTCACGGTGGCCGTTGTGTG
CCAGGCTCAGCCCAGCCGTGTCACTGTCCCCCCGGCTTCCAGGGACCCCGCTGTCAGTATGATGTGGA
CGAATGCCGAACCCACAACGGTGGCTGCCAGCACCGGTGCGTGAACACCCCAGGCTCCTACCTCTGTG
AGTGCAAGCCCGGCTTCCGGCTCCACACTGACAGCAGGACCTGCCTGGCCATTAACTCCTGCGCCCTG
GGCAATGGCGGCTGCCAGCACCACTGTGTCCAGCTCACAATCACTCGGCATCGCTGCCAGTGCCGGCC
CGGGTTCCAGCTCCAGGAGGACGGCAGGCATTGTGTCCGTAGAAGCCCGTGTGCCAACAGGAACGGCA
GCTGCATGCACAGGTGCCAGGTGGTCCGGGGCCTCGCCCGCTGTGAGTGCCACGTGGGCTATCAGCTA
GCAGCGGACGGCAAGGCCTGTGAAGATGTGGACGAATGTGCCGCAGGGCTGGCCCAGTGTGCCCATGG
CTGCCTCAACACCCAGGGGTCCTTCAAGTGCGTGTGTCACGCGGGCTATGAGCTGGGCGCCGATGGCC
GGCAGTGCTACCGTATTGAGATGGAAATCGTGAACAGCTGTGAGGCCAACAACGGCGGCTGCTCCCAT
GGCTGCAGCCACACCAGTGCTGGGCCCCTGTGCACCTGTCCCCGCGGCTACGAGCTGGACACAGATCA
GAGGACCTGCATCAGATGTCGACGACTGTGCAGACAGCCCGTGCTGCAGCAGGTGTGCACCAACAACC
CTGGCGGGTACGAGTGCGGCTGCTACGCCGGCTACCGGCTCAGTGCCGATGGCTGCGGCTGCGAGGAT
GTGGATGAGTGCGCCTCCAGCCGTGGCGGCTGCGAGCACCACTGCACCAACCTGGCCGGCTCCTTCCA
GTGCTCCTGCGAGGCCGGCTACCGGCTGCACGAGGACCGTAGGGGCTGCAGCGCCCTGGAGGAGCCGA
TGGTGGACCTGGACGGCGAGCTGCCTTTCGTGCGGCCCCTGCCCCACATTGCCGTGCTCCAGGACGAG
CTGCCGCAACTCTTCCAGGATGACGACGTCGGGGCCGATGAGGAAGAGGCAGAGTTGCGGGGCGAACA
CACGCTCACAGAGAAGTTTGTCTGCCTGGATGACTCCTTTGGCCATGACTGCAGCTTGACCTGTGATG
ACTGCAGGAACGGAGGGACCTGCCTCCTGGGCCTGGATGGCTGTGATTGCCCCGAGGGCTGGACTGGG
CTCATCTGCAATGAGAGTTGTCCTCCGGACACCTTTGGGAAGAACTGCAGCTTCTCCTGCAGCTGTCA
GAATGGTGGGACCTGCGACTCTGTCACGGGGGCCTGCCGCTGCCCCCCGGGTGTCAGTGGAACTAACT
GTGAGGATGGCTGCCCCAAGGGCTACTATGGCAAGCACTGTCGCAAGAAATGCAACTGTGCCAACCGG
GGCCGGTGCCACCGCCTCTACGGGGCCTGCCTCTGCGACCCAGGGCTCTACGGCCGCTTCTGCCACCT
CGCCTGCCCGCCGTGGGCCTTTGGGCCGGGCTGCTCGGAGGAGTGCCAGTGTGTGCAGCCCCACACGC
AGTCCTGTGACAAGAGGGATGGCAGCTGCTCCTGCAAGGCTGGCTTCCGGGGCGAGCGCTGTCAGGCA
GAGTGTGAGCCGGGCTACTTTGGGCCGGGGTGCTGGCAGGCATGCACCTGCCCAGTGGGCGTGGCCTG
TGACTCCGTGAGCGGCGAGTGTGGGAAGCGGTGTCCTGCTGGCTTCCAGGGAGAGGACTGTGGCCAAG
AGTGCCCGGTGGGGACCTTTGGCGTGAACTGCTCGAGCTCCTGCTCCTGTGGGGGGGCCCCCTGCCAC
GGGGTCACGGGGCAGTGCCGGTGTCCGCCGGGGAGGACTGGGGAAGACTGTGAGGCAGGTGAGTGTGA
GGGCCTCTGGGGGCTGGGCTGCCAGGAGATCTGCCCAGCATGCCATAACGCTGCTCGCTGCGACCCTG
AGACCGGAGCCTGCCTGTGCCTCCCTGGCTTTGTCGGCAGCCGCTGCCAGGACTGTGAGGCAGGCTGG
TATGGTCCCAGCTGCCAGACAATGTGCTCTTGTGCCAATGATGGGCACTGCCACCAAGACACGGGACA
CTGCAGCTGTGCCCCCGGGTGGACCGGCTTTAGCTGCCAGAGAGCCTGTGATACTGGGCACTGGGGAC
CTGACTGCAGCCACCCCTGCAACTGCAGCGCTGGCCACGGGAGCTGTGATGCCATCAGCGGCCTGTGT
CTGTGTGAGGCTGGCTACGTGGGCCCGCGGTGCGAGCAGTCAGAGTGTCCCCAGGGCCACTTTGGGCC
CGGCTGTGAGCAGCGGTGCCAGTGTCAGCATGGAGCAGCCTGTGACCACGTCAGCGGGGCCTGCACCT
GCCCGGCCGGCTGGAGGGGCACCTTCTGCGAGCATGCCTGCCCGGCCGGCTTCTTTGGATTGGACTGT
CGCAGTGCCTGCAACTGCACCGCCGGAGCTGCCTGTGATGCCGTGAATGGCTCCTGCCTCTGCCCCGC
TGGCCGCCGGGGCCCCCGCTGTGCCGAGAGTGCCTGCCCAGCCCACACCTACGGGCACAATTGCAGCC
AGGCCTGTGCCTGCTTTAACGGGGCCTCCTGTGACCCTGTCCACGGGCAGTGCCACTGTGCCCCTGGC
TGGATGGGGCCCTCCTGCCTGCAGGCCTGCCCTGCCGGCCTGTACGGCGACAACTGTCGGCATTCCTG
CCTCTGCCAGAACGGAGGGACCTGTGACCCTGTCTCAGGCCACTGTGCGTGCCCAGAGGGCTGGGCCG
GCCTGGCCTGTGAGGTAGAGTGCCTCCCCCGGGACGTCAGAGCTGGCTGCCGGCACAGCGGCGGTTGC
CTCAACGGGGGCCTGTGTGACCCGCACACGGGCCGCTGCCTCTGCCCAGCCGGCTGGACTGGGGACAA
GTGTCAGAGCCCTGCAGCCTGTGCCAAGGGCACATTCGGGCCTCACTGTGAGGGGCGCTGTGCCTGCC
GGTGGGGAGGCCCCTGCCACCTTGCCACCGGGGCCTGCCTCTGCCCTCCGGGGTGGCGGGGGCCTCAT
CTTTCTGCAGCCTGCCTGCGGGGCTGGTTTGGAGAGGCCTGTGCCCAGCGCTGCAGCTGCCCGCCTGG
CGCTGCCTGCCACCACGTCACTGGGGCCTGCCGCTGTCCCCCTGGCTTCACTGGCTCCGGCTGCGAGC
AGGCCTGCCCACCCGGCAGCTTTGGGGAGGACTGTGCGCAGATGTGCCAGTGTCCCGGTGAGAACCCG
GCCTGCCACCCTGCCACCGGGACCTGCTCATGTGCTGCTGGCTACCACGGCCCCAGCTGCCAGCAACG
ATGTCCGCCCGGGCGGTATGGGCCAGGCTGTGAACAGCTGTGTGGGTGTCTCAACGGGGGCTCCTGTG
ATGCGGCCACGGGGGCCTGCCGCTGCCCCACTGGGTTCCTCGGGACGGACTGCAACCTCACCTGTCCG
CAGGGCCGCTTCGGCCCCAACTGCACCCACGTGTGTGGGTGTGGGCAGGGGGCGGCCTGCGACCCTGT
GACCGGCACCTGCCTCTGCCCCCCGGGGAGAGCCGGCGTCCGCTGTGAGCGAGGCTGCCCCCAGAACC
GGTTTGGCGTGGGCTGCGAGCACACCTGCTCCTGCAGAAATGGGGGCCTGTGCCACGCCAGCAAGCGG
CAGCTGCTCCTGTGGCCTGGGCTGGACGGGGCGGCACTGCGAGCTGGCCTGTCCCCCTGGGCGCTACG
GAGCCGCCTGCCATCTGGAGTGCTCCTGCCACAACAACAGCACGTGTGAGCCTGCCACGGGCACCTGC
CGCTGCGGCCCCGGCTTCTATGGCCAGGCCTGCGAGCACCCCTGTCCCCCTGGCTTCCACGGGGCTGG
CTGCCAGGGGTTGTGCTGGTGTCAACATGGAGCCCCCTGCGACCCCATCAGTGGCCGATGCCTCTGCC
CTGCCGGCTTCCACGGCCACTTCTGTGAGAGGGGGTGTGAGCCAGGTTCATTTGGAGAGGGCTGCCAC
CAGCGCTGTGACTGTGACGGGGGGGCACCCTGTGACCCTGTCACCGGTCTCTGCCTTTGCCCACCAGG
GCGCTCAGGAGCCACCTGTAACCTGGATTGCAGAAGGGGCCAGTTTGGGCCCAGCTGCACCCTGCACT
GTGACTGCGGGGGTGGGGCTGACTGCGACCCTGTCAGTGGGCAGTGTCACTGTGTGGATGGCTACATG
GGCAACACTGCCCGCCTCCAGCAGACCCACATCCCGGAGCGGTGGACCAGCGAGGCACTAGTAGAGGC
AGTCCCGTGGAGCCCGCCTCTCCAGTCCCAGCCAGAGGGGACCCTGGCCTTTGGTGACCACTGAGAAG
GACACTTCACGGGCCCAGAGCTCCTGGTACTGCCCTTCCTTTGAGGGCCGTGGAGGGCTGTGGACAGC
CCAGCAACCTGTCGCTCTTGGAGGCTGGTGTGGCCTTGAGGAGGGAAGCCTCGCATGGCCGCTGGAAG
AGAGGCGCCTCCTGGCCTGGCTCTGCAGAACCCAGGGGCACGCTCTGGGCCTGGGCTGAGGAAGTCCC
GCTCTCCCCGCGGCTCTGAGTTGGACTGAGGACAGGTGTGGGCGCCAGTGTGGGTGCAGGCGCAGGTG
AGGCACAAGGGCCACTGTCCTCCAGGCAGGCTTTTTGGTGCTAGGCCCTGGGACTGGAAGTCGCCCAG
CCCGTATTTATGTAAAGGTATTTATGGGCCACTGCACATGCCCGCTGCAGCCCTGGGATCAGCTGGAA
GCTGCCTGTCATCTCCTGCCCAATCCCCAGAAACCCTGATTCAGGTCTGCAGGCTCCTGCGGGCTCAC
CAGGCTGCTGGCTCCGGTACCATGTAAACCTAGGAAGGTAAAGGAGCAGGCAACCTCCTCGTGGCCTG
TGTGTTTGCTGTGTTACGTGGACTCTGTGTGGGCTCCTCCCTGGGGCCCGGCCAGCATAACGGTGCAC
CCAGGGACCTCCCAGTGCACCCGGGGCCCTTTGCAGGGGTGGGGGTGCCACACAAGTGAAGAAGTTGG
GACTCATCTCAGTTCCCAGTGCTATTGAGGAGAACGCTGGGGCTGCATTCATTACCGCTGAGACCCAG
AGACTGGCTGTTCCCAGAGAATGGCCCAGGGGGAGGAGGGCTGGTGTGGAGGGGCAACCTGGACTGAG
GCCGAACTCCCTTGGGCTCACCCCACCCACCCCTACCTGAGCATCAGCAGTGGGGGGAGGGCAGCATC
GCAGGGGCAGGGACTCCCTGGGTGAGGACAGACCAGCCCTCCCGAGCACCTGGCACTCATGGGCTGAG
GCTGACTTCTCCTGGAAGAAGGGCCCAGAGTGGAAGGAAGAGGCAGAGGGTAGAGGTGGTGGCTGGGG
GCTCCTCTGCAGAGTGGGGTGGCCAATGGAGAGGGCTGCACTCACACCGCAACATAGGACTCTCTCTC
CCTTAAGAAGGCCCCCTTAGGGTCTGGGCTGCCGCCCCCATCACCCTAAAACCAGCCAAGGTAGCTGA
GGCCCCAGGGCAGACAATTTCACCAGCAGGANGAGGAGGAGTCCAGTGAGCTTGGTTGCTCACAGACA
GCAAGGGAGCTGTCACAGAGGAAGCTGATGAATGGACCGCTGTGGGGAGACTTTAAAGTAGAACAGTG
ATAAGGGAGGGCAGGATGGTGGGGATGCAGAAGCAGCAGCCAGAGAGAGACGGACTGGGGTGCAGACC
GAGTGTGGAAAACGCATACCTTGAAATGAAGCATCCAGCAGATGGGGTGAGTGGATACAGCTCAGGAG
ATTCTCCCAGGAATAGCAGGGAGGCGTAAAGAGAGACAACGTACAGAGATAGATGAATGGAAATGGGT
AAGGGAGGTGTTCATTCACATCCATCTAACTGCAAAATACAAAAGTAAGAAGTCATTGACATGAAGCA
ACGACGACCAAGACGTTCTCAGATCTAAAGGTGAATGATCTCAGTCAGCCTGGAAATGCACAAGGTGC
AAAAATAACATAAAAAAGCCATAAGACCTTGAAGAACATCAATGTCAAAGATAAATTCTAAAGTCCCA
GAGAAAAAAGAATGGGAATCAAATTGACCTCAGACTATACGTGAGAAACACGGAGAGCCAGAAAACTG
TGATGTTCCATCCTCAGAGTTTGAAGGAAATATTTGAAGGCTGAATTTTACATCCAGCTAAACTATCA
AAGGCATGCAAAGTCCATGTTATTCTTAGGCCTTCAAGGCCTCGGCCATTTTTCTACAGAAAAGCCTG
ATTTTAAAATGCTCTTAGAGACGTTCTCCAGCCAGAAGAGAAAGAAGCCAGGAGGGTGCTCTGAGATA
TTCAGTCACCACAGTTCCCAAATGGCCTAGGAATTCAGAGAGTCAGAATATCACCATTACTCCCCAAT
GGGAACCCCCGACAGTCTCAGCATGGTGTGAGGGTGTGGACGGGGGGCCTGGCAGGTACCAATCACTC
ATCCCGCTCAGTGAAGACACAGTGTTCAGCTACGGAAGCCATAAGGCAGGCCGAGCTTCTGCCCATCC
GGAGGAAATCTCAGCTATCCAACGGCGGTCAGGAGCAGAGGAAAATAAAGCAGAATAACTAGAAAACA
CGCTCACAGATCCTAATGTTAACGGTTACAAATGACGACGGAAAAACAAACTCCTGACCATATATTAT
ATAGTTTCAAGCAGCAAGAAGGAGGATATTGAACATTCTCAACACACATAATAAACGCTTGAGATGAT
GATATGCTCATTACCCTGATTTGATCACTAGACATNCCATGTATCAAAACATCACTGTGTATCCGATC
AATATCTACAATTATTGTCAATTAAAAACATCATTAAAAACAA

| | | | |
|---|---|---|---|
| NOV37h, CG56449-02 | | | |
| Protein Sequence | SEQ ID NO: 872 | 1398 aa | MW at 147208.2kD |

MPMGHSDRWSWRLLRLALPLPVWLPAGGGRGADSPCLCSRPHVCAEQELTLVGRRQPCVQALSHTVPV
WKAGCGWQAWCVGHERRTVYYMGYRQVYTTEARTVLRCCRGWTQQPDEEGCLSAECSASLCFHGGRCV
PGSAQPCHCPPGFQGPRCQYDVDECRTHNGGCQHRCVNTPGSYLCECKPGFRLHTDSRTCLAINSCAL
GNGGCQHHCVQLTITRHRCQCRPGFQLQEDGRHCVPRSPCANRNGSCMHRCQVVRGLARCECHVGYQL
AADGKACEDVDECAAGLAQCAHGCLNTQGSFKCVCHAGYELGADGRQCYRIEMEIVNSCEANNGGCSH
GCSHTSAGPLCTCPRGYELDTDQRTCIRCRRLCRQPVLQQVCTNNPGGYECGCYAGYRLSADGCGCED
VDECASSRGGCEHHCTNLAGSFQCSCEAGYRLHEDRRGCSALEEPMVDLDGELPFVRPLPHIAVLQDE
LPQLFQDDDVGADEEEAELRGEHTLTEKFVCLDDSFGHDCSLTCDDCRNGGTCLLGLDGCDCPEGWTG
LICNESCPPDTFGKNCSFSCSCQNGGTCDSVTGACRCPPGVSGTNCEDGCPKGYYGKHCRKKCNCANR
GRCHRLYGACLCDPGLYGRFCHLACPPWAFGPGCSEECQCVQPHTQSCDKRDGSCSCKAGFRGERCQA
ECEPGYFGPGCWQACTCPVGVACDSVSGECGKRCPAGFQGEDCGQECPVGTFGVNCSSSCSCGGAPCH
GVTGQCRCPPGRTGEDCEAGECEGLWGLGCQEICPACHNAARCDPETGACLCLPGFVGSRCQDCEAGW
YGPSCQTMCSCANDGHCHQDTGHCSCAPGWTGFSCQRACDTGHWGPDCSHPCNCSAGHGSCDAISGLC
LCEAGYVGPRCEQSECPQGHFGPGCEQRCQCQHGAACDHTSGACTCPAGWRGTFCEHACPAGFFGLDC
RSACNCTAGAACDAVNGSCLCPAGRRGPRCAESACPAHTYGHNCSQACACFNGASCDPVHGQCHCAPG
WMGPSCLQACPAGLYGDNCRHSCLCQNGGTCDPVSGHCACPEGWAGLACEVECLPRDVRAGCRHSGGC
LNGGLCDPHTGRCLCPAGWTGDKCQSPAACAKGTFGPFCEGRCACRWGGPCHLATGACLCPPGWRGPH
LSAACLRGWFGEACAQRCSCPPGAACHHVTGACRCPPGFTGSGCEQACPPGSFGEDCAQMCQCPGENP
ACHPATGTCSCAAGYHGPSCQQRCPPGRYGPGCEQLCGCLNGGSCDAATGACRCPTGFLGTDCNLTCP
QGRFGPNCTHCGCGQGAACDPVTGTCLCPPGRAGVRCERGCPQNRFGVGCEHTCSCRNGGLCHASKR
QLLLWPGLDGAALRAGLSPWALRSRLPSGVLLPQQQHV

| | | |
|---|---|---|
| NOV37i, CG56449-03 | SEQ ID NO: 873 | 4733 bp |
| DNA Sequence | ORF Start: ATG at 1 | ORF Stop: TAG at 4351 |

ATGCCCATGGGACATTCTGACAGGTGGTCTTGGCGTCTCCTGAGGCTGGCACTGCCACTCCCAGTCTG
GTTGCCGGCTGGGGGTGGCCGAGGCGCTGACTCTCCATGTCTCTGTTCCAGGCCCCACGTGTGTGCTG
TGGAAGGCCGGCTGTGGGTGGCAGGCGTGGTGCGTGGGTCATGAGCGGAGAACCGTCTACTACATGGG
CTACAGGCAGGTGTATACCACGGAGGCCCGGACCGTGCTCAGGTGCTGCCGAGGGTGGACGCAGCAGC
CCGACGAGGAGGGCTGCCTCTCGGCTGAATGCAGCGCCAGCCTCTGTTTTCACGGTGGCCGTTGTGTG
CCAGGCTCAGCCCAGCCGTGTCACTGTCCCCCCGGCTTCCAGGGACCCCGCTGTCAGTATGATGTGGA
CGAATGCCGAACCCACAACGGTGGCTGCCAGCACCGGTGCGTGAACACCCCAGGCTCCTACCTCTGTG
AGTGCAAGCCCGGCTTCCGGCTCCACACTGACAGCAGGACCTGCCTGGCCATTAACTCCTGCGCCCTG
GGCAATGGCGGCTGCCAGCACCACTGTGTCCAGCTCACAATCACTCGGCATCGCTGCCAGTGCCGGCC
CGGGTTCCAGCTCCAGGAGGACGGCAGGCATTGTGTCCGTAGAAGCCCGTGTGCCAACAGGAACGGCA
GCTGCATGCACAGGTGCCAGGTGGTCCGGGGCCTCGCCCGCTGTGAGTGCCACGTGGGCTATCAGCTA
GCAGCGGACGGCAAGGCCTGTGAAGATGTGGACGAATGTGCCGCAGGGCTGGCCCAGTGTGCCCATGG
CTGCCTCAACACCCAGGGGTCCTTCAAGTGCGTGTGTCACGCGGGCTATGAGCTGGGCGCCGATGGCC
GGCAGTGCTACCGTATTGAGATGGAAATCGTGAACAGCTGTGAGGCCAACAACGGCGGCTGCTCCCAT
GGCTGCAGCCACACCAGTGCTGGGCCCCTGTGCACCTGTCCCCGCGGCTACGAGCTGGACACAGATCA
GAGGACCTGCATCAGATGTCGACGACTGTGCAGACAGCCCGTGCTGCAGCAGGTGTGCACCAACAACC
CTGGCGGGTACGAGTGCGGCTGCTACGCCGGCTACCGGCTCAGTGCCGATGGCTGCGGCTGCGAGGAT
GTGGATGAGTGCGCCTCCAGCCGTGGCGGCTGCGAGCACCACTGCACCAACCTGGCCGGCTCCTTCCA
GTGCTCCTGCGAGGCCGGCTACCGGCTGCACGAGGACCGTAGGGGCTGCAGCGCCCTGGAGGAGCCGA
TGGTGGACCTGGACGGCGAGCTGCCTTTCGTGCGGCCCCTGCCCCACATTGCCGTGCTCCAGGACGAG
CTGCCGCAACTCTTCCAGGATGACGACGTCGGGGCCGATGAGGAAGAGGCAGAGTTGCGGGGCGAACA
CACGCTCACAGAGAAGTTTGTCTGCCTGGATGACTCCTTTGGCCATGACTGCAGCTTGACCTGTGATG
ACTGCAGGAACGGAGGGACCTGCCTCCTGGGCCTGGATGGCTGTGATTGCCCCGAGGGCTGGACTGGG
CTCATCTGCAATGAGAGTTGTCCTCCGGACACCTTTGGGAAGAACTGCAGCTTCTCCTGCAGCTGTCA
GAATGGTGGGACCTGCGACTCTGTCACGGGGGCCTGCCGCTGCCCCCCGGGTGTCAGTGGAACTAACT
GTGAGGATGGCTGCCCCAAGGGCTACTATGGCAAGCACTGTCGCAAGAAATGCAACTGTGCCAACCGG
GGCCGGTGCCACCGCCTCTACGGGGCCTGCCTCTGCGACCCAGGGCTCTACGGCCGCTTCTGCCACCT
CGCCTGCCCGCCGTGGGCCTTTGGGCCGGGCTGCTCGGAGGAGTGCCAGTGTGTGCAGCCCCACACGC
AGTCCTGTGACAAGAGGGATGGCAGCTGCTCCTGCAAGGCTGGCTTCCGGGGCGAGCGCTGTCAGGCA
GAGTGTGAGCTGGGCTACTTTGGGCCGGGGTGCTGGCAGGCATGCACCTGCCCAGTGGGCGTGGCCTG
TGACTCCGTGAGCGGCGAGTGTGGGAAGCGGTGTCCTGCTGGCTTCCAGGGAGAGGACTGTGGCCAAG
AGTGCCCGGTGGGGACGTTTGGCGTGAACTGCTCGAGCTCCTGCTCCTGTGGGGGGGCCCCCTGCCAC
GGGGTCACGGGGCAGTGCCGGTGTCCACCGGGGAGGACTGGGGAAGACTGTGAGGCAGATTGTCCCGA
GGGCCGCTGGGGGCTGGGCTGCCAGGAGATCTGCCCAGCATGCCAGCACGCTGCCCGCTGCGACCCTG
AGACCGGAGCCTGCCTGTGCCTCCCTGGCTTCGTCGGCAGCCGCTGCCAGGACGTGTGCCCAGCAGGC
TGGTATGGTCCCAGCTGCCAGACAAGGTGCTCTTGTGCCAATGATGGGCACTGCCACCCAGCCACCGG
ACACTGCAGCTGTGCCCCCGGGTGGACCGGCTTTAGCTGCCAGAGAGCCTGTGATACTGGGCACTGGG
GACCTGACTGCAGCCACCCCTGCAACTGCAGCGCTGGCCACGGGAGCTGTGATGCCATCAGCGGCCTG
TGTCTGTGTGAGGCTGGCTACGTGGGCCCGCGGTGCGAGCAGCAGTGTCCCCAGGGCCACTTTGGGCC
CGGCTGTGAGCAGCTGTGCCAGTGTCAGCATGGAGCAGCCTGTGACCACGTCAGCGGGGCCTGCACCT
GCCCGGCCGGCTGGAGGGGCACCTTCTGCGAGCATGCCTGCCCGGCCGGCTTCTTTGGATTGGACTGT
CGTAGTGCCTGCAACTGCACCGCCGGAGCTGCCTGTGATGCCGTGAATGGCTCCTGCCTCTGCCCCGC
TGGCCGCCGGGGCCCCCGCTGTGCCGAGACCTGCCCTGCCGGCCTGTACGGCGACAACTGTCGGCATT
CCTGCCTCTGCCAGAACGGAGGGACCTGTGACCCTGTCTCAGGCCACTGTGCGTGCCCAGAGGGCTGG
GCCGGCCTGGCCTGTGAGAAGGAGTGCCCCCCCCGGGACGTCAGAGCTGGCTGCCGGCACAGCGGTGG
TTGCCTCAACGGGGGCCTGTGTGACCCGCACACGGGCCGCTGCCTCTGCCCAGCCGGCTGGGCTGGGG
ACAAGTGTCAGAGCCCCTGCCTGCGGGGCTGGTTTGGAGAGGCCTGTGCCCAGCACTGCAGCTGCCCG
CCTGGCGCTGCCTGCCACCACGTCACTGGGGCCTGCCGCTGTCCCCCTGGCTTCACTGGCTCCGGCTG
CGAGCAGGGATGTCCGCCCGGGCGGTATGGGCCAGGCTGTGAACAGCTGTGTGGGTGTCTCAACGGGG
GCTCCTGTGATGCGGCCACGGGGGCCTGCCGCTGCCCCACTGGGTTCCTCGGGACGGACTGCAACCTC
ACCTGTCCGCAGGGCCGCTTCGGCCCCAACTGCACCCACGTGTGTGGGTGTGGGCAGGGGGCGGCCTG
CGACCCTGTGACCGGCACCTGCCTCTGCCCCCCGGGGAGAGCCGGCGTCCGCTGTGAGCGAGGCTGCC
CCCAGAACCGGTTTGGCGTGGGCTGCGAGCACACCTGCTCCTGCAGAAATGGGGGCCTGTGCCACGCC
AGCAACGGCAGCTGCTCCTGTGGCCTGGGCTGGACGGGGCGGCACTGCGAGCTGGCCTGTCCCCCTGG
GCGCTACGGAGCCGCCTGCCATCTGGAGTGCTCCTGCCACAACAACAGCACGGGTGAGCCTGCCACGG
GCACCTGCCGCTGCGGCCCCGGCTTCTATGGCCAGGCCTGCGAGCACCCCTGTCCCCCTGGCTTCCAC
GGGGCTGGCTGCCAGGGGTTGTGCTGGTGTCAACATGGAGCCCCCTGCGACCCCATCAGTGGCCGATG
CCTCTGCCCTGCCGGCTTCCACGGCCACTTCTGTGAGAGGGGGTGTGAGCCAGGTTCATTTGGAGAGG
GCTGCCACCAGCGCTGTGACTGTGACGGGGGGGCACCCTGTGACCCTGTCACCGGTCTCTGCCTTTGC
CCACCAGGGCGCTCAGGAGCCACCTGTAACCTGGATTGCAGAAGGGGCCAGTTTGGGCCCAGCTGCAC
CCTGCACTGTGACTGCGGGGGTGGGGCTGACTGCGACCCTGTCAGTGGGCAGTGTCACTGTGTGGATG
GCTACATGGGGCCCACGTGCCGGGAAGGTGGGCCCCTCCGGCTCCCCGAGAACCCGTCCTTAGCCCAG
GGCTCAGCGGGCACACTGCCCGCCTCCAGCAGACCCACATCCCGGAGCGGTGGACCAGCGAGGCACTA
GTAGAGGCAGTCCCGTGGAGCCCGCCTCTCCAGTCCCAGCCAGAGGGGACCCTGGCCTTTGGTGACCA
CTGAGAAGGACACTTCACGGGCCCAGAGCTCCTGGTACTGCCCTTCCTTTGAGGGCCGTGGAGGGCTC
TGGACAGCCCAGCAACCTGTCGCTCTTGGAGGCTGGTGTGGCCTTGAGGAGGGAAGCCTCGCATGGCC
GCTGGAAGAGAGGCGCCTCCTGGCCTGGCTCTGCAGAACCCAGGGGCACGCTCTGGGCCTGGGCTGAG
GAAGTCCCGCTCTCCCCGCGGCTCTGAGTTGGACTGAGGACAGGTGTGGGCGCCAGTGTGGGTGCAGG
CGCAGGTGCAGGCACAGGGCCACTGTCCTCCAGGCAGGCTT

| | | | |
|---|---|---|---|
| NOV37i, CG56449-03 | | | |
| Protein Sequence | SEQ ID NO: 874 | 1450 aa | MW at 152213.4kD |

MPMGHSDRWSWRLLRLALPLPVWLPAGGGRGADSPCLCSRPHVCAEQELTLVGRRQPCVQALSHTVPV
WKAGCGWQAWCVGHERRTVYYMGYRQVYTTEARTVLRCCRGWTQQPDEEGCLSAECSASLCFHGGRCV
PGSAQPCHCPPGFQGPRCQYDVDECRTHNGGCQHRCVNTPGSYLCECKPGFRLHTDSRTCLAINSCAL
GNGGCQHHCVQLTITRHRCQCRPGFQLQEDGRHCVRRSPCANRNGSCMHRCQVVRGILACECHVGYQL
AADGKACEDVDECAAGLAQCAHGCLNTQGSFKCVCHAGYELGADGRQCYRIEMEIVNSCEANNGGCSH
GCSHTSAGPLCTCPRGYELDTDQRTCIRCRRLCRQPVLQQVCTNNPGGYECGCYAGYRLSADGCGCED
VDECASSRGGCEHHCTNLAGSFQCSCEAGYRLHEDRRGCSALEEPMVDLDGELPFVRPLPHIAVLQDE
LPQLFQDDDVGADEEEAELRGEHTLTEKFVCLDDSFGHDCSLTCDDCRNGGTCLLGLDGCDCPEGWTG
LICNESCPPDTFGKNCSFSCSCQNGGTCDSVTGACRCPPGVSGTNCEDGCPKGYYGKHCRKKCNCANR
GRCHRLYGACLCDPGLYGRFCHLACPPWAFGPGCSEECQCVQPHTQSCDKRDGSCSCKAGFRGERCQA
ECELGYFGPGCWQACTCPVGVACDSVSGECGKRCPAGFQGEDCGQECPVGTFGVNCSSSCSCGGAPCH
GVTGQCRCPPGRTGEDCEADCPEGRWGLGCQEICPACQHAARCDPETGACLCLPGFVGSRCQDVCPAG
WYGPSCQTRCSCANDGHCHPATGHCSCAPGWTGFSCQRACDTGHWGPDCSHPCNCSAGHGSCDAISGL
CLCEAGYVGPRCEQQCPQGHFGPGCEQLCQCQHGAACDHVSGACTCPAGWRGTFCEHACPAGFFGLDC
RSACNCTAGAACDAVNGSCLCPAGRRGPRCAETCPAGLYGDNCRHSCLCQNGGTCDPVSGHCACPEGW
AGLLACEKECPPRDVPAGCRHSGGCLNGGCDPHTGRCLCPAGWAGDKCQSPCLRGWFGEACAQHCSCP
PGAACHHVTGACRCPPGFTGSGCEQGCPPGRYGPGCEQLCGCLNGGSCDAATGACRCPTGFLGTDCNL
TCPQGRFGPNCTHVCGCGQGAACDPVTGTCLCPPGRAGVRCERGCPQNRFGVGCEHTCSCRNGGLCHA
SNGSCSCGLGWTGRHCELACPPGRYGAACHLECSCHNNSTGEPATGTCRCGPGFYGQACEHPCPPGFH
GAGCQGLCWCQHGAPCDPISGRCLCPAGFHGHFCERGCEPGSFGEGCHQRCDCDGGAPCDPVTGLCLC
PPGRSGATCNLDCRRGQFGPSCTLHCDCGGGADCDPVSGQCNCVDGYMGPTCREGGPLRLPENPSLAQ
GSAGTLPASSRPTSRSGGPARH

| | | |
|---|---|---|
| NOV37j, CG56449-05 | SEQ ID NO: 875 | 522 bp |
| DNA Sequence | ORF Start: ATG at 29 | ORF Stop: at 515 |

GGATCCGTGCCTCGCTGGTCCACCGCTCATGGCTCCTGCCTCTGCCCCGCTGGCCGCCGGGGCCCCCG
CTGTGCCGAGACCTGCCCTGCCGGCCTGTACGGCGACAACTGTCGGCATTCCTGCCTCTGCCAGAACG
GAGGGACCTGTGACCCTGTCTCAGGCCTGCGAGCACCCCTGTCCCCCTGGCTTCCACGGGGCTGGCTG
CCAGGGGTTGTGCTGGTGTCAACATGGAGCCCCCTGCGACCCCATCAGTGGCCGATGCCTCTGCCCTG
CCGGCTTCCACGGCCACTTCTGTGAGAGGGATTGCAGAAGGGGCCAGTTTGGGCCCAGCTGCACCCTG
CACTGTGACTGCGGGGGTGGGGCTGACTGCGACCCTGTCAGTGGGCAGTGTCACTGTGTGGATGGCTA
CATGGGGCCCACGTGCCGGGAAGGTGGGCCCCTCCGGCTCCCCGAGAACCCGTCCTTAGCCCAGGGCT
CAGCGGGCACACTGCCCGCCTCCAGCAGACCCACATCCCGCTCGAG

| | | | |
|---|---|---|---|
| NOV37l, CG56449-05 | | | |
| Protein Sequence | SEQ ID NO: 876 | 162 aa | MW at 16251.2kD |

MAPASAPLAAGAPAVPRPALPACTATTVGIPASARTEGPVTLSQACEHPCPPGFHGAGCQGLCWCQHG
APCDPISGRCLCPAGFHGHFCERDCRRGQFGPSCTLHCDCGGGADCDPVSGQCHCVDGYMGPTCREGG
PLRLPENPSLAQGSAGTLPASSRPTS

| | | |
|---|---|---|
| NOV37k, CG56449-06 | SEQ ID NO: 877 | 7334 bp |
| DNA Sequence | ORF Start: ATG at 1 | ORF Stop: TGA at 4210 |

ATGCCCATGGGACATTCTGACAGGTGGTCTTGGCGTCTCCTGAGGCTGGCACTGCCACTCCCAGTCTG
GTTGCCGGCTGGGGGTGGCCGAGGCGCTGACTCTCCATGTCTCTGTTCCAGGCCCCACGTGTGTGCTG
AGCAGGAGCTGACCCTGGTGGGCCGCCGCCAGCCGTGCGTGCAGGCCTTAAGCCACACGGTGCCGGTG
TGGAAGGCCGGCTGTGGGTGGCAGGCGTGGTGCGTGGGTCATGAGCGGAGGACCGTCTACTACATGGG
CTACAGGCAGGTGTATACCACGGAGGCCCGGACCGTGCTCAGGTGCTGCCGAGGGTGGATGCAGCAGC
CCGACGAGGAGGGCTGCCTCTCGGATGTGGGTGAGTGTGCCAACGCCAACGGGGGCTGTGCGGGTCGG
TGCCGGGACACCGTGGGGGGCTTCTACTGCCGCTGGCCCCCCCCCAGCCACCAGCTGCAGGGTGATGG
CGAGACTTGCCAAGATGTGGACGAATGCCGAACCCACAACGGTGGCTGCCAGCACCGGTGCGTGAACA
CCCCAGGCTCCTACCTCTGTGAGTGCAAGCCCGGCTTCCGGCTCCACACTGACAGCAGGACCTGCGCC
ATTAACTCCTGCGCCCTGGGCAATGGCGGCTGCCAGCACCACTGTGTCCAGCTCACAATCACTCGGCA
TCGCTGCCAGTGCCGGCCCGGGTTCCAGCTCCAGGAGGACGGCAGGCATTGTGTCCGTAGAAGCCCGT
GTGCCAACAGGAACGGCAGCTGCATGCACAGGTGCCAGGTGGTCCGGGGCCTCGCCCGCTGTGAGTGC
CACGTGGGCTATCAGCTAGCAGCGGACGGCAAGGCCTGTGAAGATGTGGACGAATGTGCCGCAGGGCT
GGCCCAGTGTGCCCATGGCTGCCTCAACACCCAGGGGTCCTTCAAGTGCGTGTGTCACGCGGGCTATG
AGCTGGGCGCCGATGGCCGGCAGTGCTACCGTATTGAGATGGAAATCGTGAACAGCTGTGAGGCCAAC
AACGGCGGCTGCTCCCATGGCTGCAGCCACACCAGTGCTGGGCCCCTGTGCACCTGTCCCCGCGGCTA
CGAGCTGGACACAGATCAGAGGACCTGCATCAGATGTCGACGACTGTGCAGACAGCCCGTGCTGCAGC
AGGTGTGCACCAACAACCCTGGCGGGTACGAGTGCGGCTGCTACGCCGGCTACCGGCTCAGTGCCGAT
GGCTGCGGCTGCGAGGATGTGGATGAGTGCGCCTCCAGCCGTGGCGGCTGCGAGCACCACTGCACCAA
CCTGGCCGGCTCCTTCCAGTGCTCCTGCGAGGCCGGCTACCGGCTGCACGAGGACCGTAGGGGCTGCA
GCGCCCTGGAGGAGCCGATGGTGGACCTGGACGGCGAGCTGCCTTTCGTGCGGCCCCTGCCCCACATT
GCCGTGCTCCAGGACGAGCTGCCGCAACTCTTCCAGGATGACGACGTCGGGGCCGATGAGGAAGAGGC
AGAGTTGCGGGGCGAACACACGCTCACAGAGAAGTTTGTCTGCCTGGATGACTCCTTTGGCCATGACT
GCAGCTTGACCTGTGATGACTGCAGGAACGGAGGGACCTGCCTCCTGGGCCTGGATGGCTGTGATTGC
CCCGAGGGCTGGACTGGGCTCATCTGCAATGAGAGTTGTCCTCCGGACACCTTTGGGAAGAACTGCAG
CTTCTCCTGCAGCTGTCAGAATGGTGGGACCTGCGACTCTGTCACGGGGGCCTGCCGCTGCCCCCCGG
GTGTCAGTGGAACTAACTGTGAGGATGGCTGCCCCAAGGGCTACTATGGCAAGCACTGTCGCAAGAAA
TGCAACTGTGCCAACCGGGGCCGGTGCCACCGCCTCTACGGGGCCTGCCTCTGCGACCCAGGGCTCTA
CGGCCGCTTCTGCCACCTCGCCTGCCCGCCGTGGGCCTTTGGGCCGGGCTGCTCGGAGGAGTGCCAGT
GTGTGCAGCCCCACACGCAGTCCTGTGACAAGAGGGATGGCAGCTGCTCCTGCAAGGCTGGCTTCCGG
GGCGAGCGCTGTCAGGCAGAGTGTGAGCCGGGCTACTTTGGGCCGGGGTGCTGGCAGGCATGCACCTG
CCCAGTGGGCGTGGCCTGTGACTCCGTGAGCGGCGAGTGTGGGAAGCGGTGTCCTGCTGGCTTCCAGG
GAGAGGACTGTGGCCAAGAGTGCCCGGTGGGGACCTTTGGCGTGAACTGCTCGAGCTCCTGCTCCTGT
GGGGGGGCCCCCTGCCACGGGGTCACGGGGCAGTGCCGGTGTCCGCCGGGGAGGACTGGGGAAGACTG
TGAGGCAGGTGAGTGTGAGGGCCTCTGGGGGCTGGGCTGCCAGGAGATCTGCCCAGCATGCCATAACG
CTGCTCGCTGCGACCCTGAGACCGGAGCCTGCCTGTGCCTCCCTGGCTTTGTCGGCAGCCGCTGCCAG
GACTGTGAGGCAGGCTGGTATGGTCCCAGCTGCCAGACAATGTGCTCTTGTGCCAATGATGGGCACTG
CCACCAAGACACGGGACACTGCAGCTGTGCCCCCGGGTGGACCGGCTTTAGCTGCCAGAGAGCCTGTG
ATACTGGGCACTGGGGACCTGACTGCAGCCACCCCTGCAACTGCAGCGCTGGCCACGGGAGCTGTGAT
GCCATCAGCGGCCTGTGTCTGTGTGAGGCTGGCTACGTGGGCCCGCGGTGCGAGCAGTCAGAGTGTCC
CCAGGGCCACTTTGGGCCCGGCTGTGAGCAGCGGTGCCAGTGTCAGCATGGAGCAGCCTGTGACCACG
TCAGCGGGGCCTGCACCTGCCCGGCCGGCTGGAGGGGCACCTTCTGCGAGCATGCCTGCCCGGCCGGC
TTCTTTGGATTGGACTGTCGCAGTGCCTGCAACTGCACCGCCGGAGCTGCCTGTGATGCCGTGAATGG
CTCCTGCCTCTGCCCCGCTGGCCGCCGGGGCCCCCGCTGTGCCGAGACCTGCCCAGCCCACACCTACG
GGCACAATTGCAGCCAGGCCTGTGCCTGCTTTAACGGGGCCTCCTGTGACCCTGTCCACGGGCAGTGC
CACTGTGCCCCTGGCTGGATGGGGCCCTCCTGCCTGCAGGCCTGCCCTGCCGGCCTGTACGGCGACAA
CTGTCGGCATTCCTGCCTCTGCCAGAACGGAGGGACCTGTGACCCTGTCTCAGGCCACTGTGCGTGCC
CAGAGGGCTGGGCCGGCCTGGCCTGTGAGGTAGAGTGCCTCCCCCGGGACGTCAGAGCTGGCTGCCGG
CACAGCGGCGGTTGCCTCAACGGGGGCCTGTGTGACCCGCACACGGGCCGCTGCCTCTGCCCAGCCGG
CTGGACTGGGGACAAGTGTCAGAGCCCTGCAGCCTGTGCCAAGGGCACATTCGGGCCTCACTGTGAGG
GGCGCTGTGCCTGCCGGTGGGGAGGCCCCTGCCACCTTGCCACCGGGGCCTGCCTCTGCCCTCCGGGG
TGGCGGGGGCCTCATCTTTCTGCAGCCTGCCTGCGGGGCTGGTTTGGAGAGGCCTGTGCCCAGCGCTG
CAGCTGCCCGCCTGGCGCTGCCTGCCACCACGTCACTGGGGCCTGCCGCTGTCCCCCTGGCTTCACTG
GCTCCGGCTGCGAGCAGGCCTGCCCACCCGGCAGCTTTGGGGAGGACTGTGCGCAGATGTGCCAGTGT
CCCGGTGAGAACCCGGCCTGCCACCCTGCCACCGGGACCTGCTCATGTGCTGCTGGCTACCACGGCCC
CAGCTGCCAGCAACGATGTCCGCCCGGGCGGTATGGGCCAGGCTGTGAACAGCTGTGTGGGTGTCTCA
ACGGGGGCTCCTGTGATGCGGCCACGGGGGCCTGCCGCTGCCCCACTGGGTTCCTCGGGACGGACTGC
AACCTCACCTGTCCGCAGGGCCGCTTCGGCCCCAACTGCACCCACGTGTGTGGGTGTGGGCAGGGGGC
GGCCTGCGACCCTGTGACCGGCACCTGCCTCTGCCCCCCGGGGAGAGCCGGCGTCCGCTGTGAGCGAG
GCTGCCCCCAGAACCGGTTTGGCGTGGGCTGCGAGCACACCTGCTCCTGCAGAAATGGGGGCCTGTGC
CACGCCAGCAAGCGGCAGCTGCTCCTGTGGCCTGGGCTGGACGGGGCGGCACTGCGAGCTGGCCTGTC
CCCCTGGGCGCTACGGAGCCGCCTGCCATCTGGAGTGCTCCTGCCACAACAACAGCACGTGTGAGCCT
GCCACGGGCACCTGCCGCTGCGGCCCCGGCTTCTATGGCCAGGCCTGCGAGCACCCCTGTCCCCCTGG
CTTCCACGGGGCTGGCTGCCAGGGGTTGTGCTGGTGTCAACATGGAGCCCCCTGCGACCCCATCAGTG
GCCGATGCCTCTGCCCTGCCGGCTTCCACGGCCACTTCTGTGAGAGGGGGTGTGAGCCAGGTTCATTT
GGAGAGGGCTGCCACCAGCGCTGTGACTGTGACGGGGGGGCACCCTGTGACCCTGTCACCGGTCTCTG
CCTTTGCCCACCAGGGCGCTCAGGAGCCACCTGTAACCTGGATTGCAGAAGGGGCCAGTTTGGGCCCA
GCTGCACCCTGCACTGTGACTGCGGGGGTGGGGCTGACTGCGACCCTGTCAGTGGGCAGTGTCACTGT
GTGGATGGCTACATGGGGCCCACGTGCCGGGAAGGTGGGCCCCTCCGGCTCCCCGAGAACCCGTCCTT
AGCCCAGGGCTCAGCGGGCACACTGCCCGCCTCCAGCAGACCCACATCCCGGAGCGGTGGACCAGCGA
GGCACTAGTAGAGGCAGTCCCGTGGAGCCCGCCTCTCCAGTCCCAGCCAGAGGGGACCCTGGCCTTTG
GTGACCACTGAGAAGGACACTTCACGGGCCCAGAGCTCCTGGTACTGCCCTTCCTTTGAGGGCCGTGG
AGGGCTGTGGACAGCCCAGCAACCTGTCGCTCTTGGAGGCTGGTGTGGCCTTGAGGAGGGAAGCCTCG
CATGGCCGCTGQAAGAGAGGCGCCTCCTGGCCTGGCTCTGCAGAACCCAGGGGCACGCTCTGGGCCTG
GGCTGAGGAAGTCCCGCTCTCCCCGCGGCTCTGAGTTGGACTGAGGACAGGTGTGGGCGCCAGTGTGG
GTGCAGGCGCAGGTGCAGGCACAGGGCCACTGTCCTCCAGGCAGGCTTTTTGGTGCTAGGCCCTGGGA
CTGGAAGTCGCCCAGCCCGTATTTATGTAAAGGTATTTATGGGCCACTGCACATGCCCGCTGCAGCCC
TGGGATCAGCTGGAAGCTGCCTGTCATCTCCTGCCCAATCCCCAGAAACCCTGATTCAGGTCTGCAGG
CTCCTGCGGGCTCACCAGGCTGCTGGCTCCGGTACCATGTAAACCTAGGAAGGTAAAGGAGCAGGCAA
CCTCCTCGTGGCCTGTGTGTTTGCTGTGTTACGTGGACTCTGTGTGGGCTCCTCCCTGGGGCCCGGCC
AGCATAACGGTGCACCCAGGGACCTCCCAGTGCACCCGGGGCCCTTTGCAGGGGTGGGGGTGCCACAC
AAGTGAAGAAGTTGGGACTCATCTCAGTTCCCAGTGCTATTGAGGAGAACGCTGGGGCTGCATTCATT
ACCGCTGAGACCCAGAGACTGGCTGTTCCCAGAGAATGGCCCAGGGGGAGGAGGGCTGGTGTGGAGGC
GCAACCTGGACTGAGGCCGAACTCCCTTGGGCTCACCCCACCCACCCCTACCTGAGCATCAGCAGTGG
GGGGAGGGCAGCATCGCAGGGGCAGGGACTCCCTGGGTGAGGACAGACCAGCCCTCCCGAGCACCTGG
CACTCATGGGCTGAGGCTGACTTCTCCTGGAAGAAGGGCCCAGAGTGGAAGGAAGAGGCAGAGGGTAG
AGGTGGTGGCTGGGGGCTCCTCTGCAGAGTGGGGTGGCCAATGGAGAGGGCTGCACTCACACCGCAAC
ATAGGACTCTCTCTCCCTTAAGAAGGCCCCCTTAGGGTCTGGGCTGCCGCCCCCATCACCCTAAAACC
AGCCAAGGTAGCTGAGGCCCCAGGGCAGACAATTTCACCAGCAGGANGAGGAGGAGTCCAGTGAGCTT
GGTTGCTCACAGACAGCAAGGGAGCTGTCACAGAGGAAGCTGATGAATGGACCGCTGTGGGGAGACTT
TAAAGTAGAACAGTGATAAGGGAGGGCAGGATGGTGGGGATGCAGAAGCAGCAGCCAGAGAGAGACGC
ACTGGGGTGCAGACGGAGTGTGGAAAACGCATACCTTGAAATGAAGCATCCAGCAGATGGGGTGAGTC
GATACAGCTCAGGAGATTCTCCCAGGAATAGCAGGGAGGCGTAAAGAGAGACAACGTACAGAGATAGA
TGAATGGAAATGGGTAAGGGAGGTGTTCATTCACATCCATCTAACTGCAAAATACAAAAGTAAGAAGT
CATTGACATGAAGCAACGACGACCAAGACGTTCTCAGATCTAAAGGTGAATGATCTCAGTCAGCCTGG
AAATGCACAAGGTGGAAAAATAACATAAAAAAGCCATAAGACCTTGAAGAACATCAATGTCAAAGATA
AATTCTAAAGTCCCAGAGAAAAAAGAATGGGAATCAAATTGACCTCAGACTATACGTGAGAAACACGG
AGAGCCAGAAAACTGTGATGTTCCATCCTCAGAGTTTGAAGGAAATATTTGAAGGCTGAATTTTACAT
CCAGCTAAACTATCAAAGGCATGCAAAGTCCATGTTATTCTTAGGCCTTCAAGGCCTCGGCCATTTTT
CTACAGAAAAGCCTGATTTTAAAATGCTCTTAGAGACGTTCTCCAGCCAGAAGAGAAAGAAGCCAGGA
GGGTGCTCTGAGATATTCAGTCACCACAGTTCCCAAATGGCCTAGGAATTCAGAGAGTCAGAATATCA
CCATTACTCCCCAATGGGAACCCCCGACAGTCTCAGCATGGTGTGAGGGTGTGGACGGGGGGCCTGGC
AGGTACCAATCACTCATCCCGCTCAGTGAAGACACAGTGTTCAGCTACGGAAGCCATAAGGCAGGCCG
AGCTTCTGCCCATCCGGAGGAAATCTCAGCTATCCAACGGCGGTCAGGAGCAGAGGAAAATAAAGCAG
AATAACTAGAAAACACGCTCACAGATCCTAATGTTAACGGTTACAAATGACGACGGAAAAACAAACTC
CTGACCATATATTATATAGTTTCAAGCAGCAAGAAGGAGGATATTGAACATTCTCAACACACATAATA
AACGCTTGAGATGATGATATGCTCATTACCCTGATTTGATCACTAGACATACCATGTATCAAAACATC
ACTGTGTATCCGATGAATATCTACAATTATTGTCAATTAAAAACATCATTAAAAACAA

| | | | |
|---|---|---|---|
| NOV37k, CG56449-06 | | | |
| Protein Sequence | SEQ ID NO: 878 | 1403 aa | MW at 147829.8kD |

MPMGHSDRWSWRLLRLALPLPVWLPAGGGRGADSPCLCSRPHVCAEQELTLVGRRQPCVQALSHTVPV
WKAGCGWQAWCVGHERRTVYYMGYRQVYTTEARTVLRCCRGWMQQPDEEGCLSDVGECANANGGCAGR
CRDTVGGFYCRWPPPSHQLQGDGETCQDVDECRTHNGGCQHRCVNTPGSYLCECKPGFRLHTDSRTCA
INSCALGNGGCQHHCVQLTITRHRCQCRPGFQLQEDGRHCVRRSPCANRNGSCMHRCQVVRGIARCEC
HVGYQLAADGKACEDVDECAAGLAQCAHGCLNTQGSFKCVCHAGYELGADGRQCYRIEMEIVNSCEAI
NGGCSHGCSHTSAGPLCTCPRGYELDTDQRTCIRCRRLCRQPVLQQVCTNNPGGYECGCYAGYRLSAD
GCGCEDVDECASSRGGCEHHCTNLAGSFQCSCEAGYRLHEDRRGCSALEEPMVDLDGELPFVRPLPHI
AVLQDELPQLFQDDDVGADEEEAELRGEHTLTEKFVCLDDSFGHDCSLTCDDCRNGGTCLLGLDGCDC
PEGWTGLICNESCPPDTFGKNCSFSCSCQNGGTCDSVTGACRCPPGVSGTNCEDGCPKGYYGKHCRKK
CNCANRGRCHRLYGACLCDPGLYGRFCHLACPPWAFGPGCSEECQCVQPHTQSCDKRDGSCSCKAGFR
GERCQAECEPGYFGPGCWQACTCPVGVACDSVSGECGKRCPAGFQGEDCGQECPVGTFGVNCSSSCSC
GGAPCHGVTGQCRCPPGRTGEDCEAGECEGLWGLGCQEICPACHNAARCDPETGACLCLPGFVGSRCQ
DCEAGWYGPSCQTMCSCANDGHCHQDTGHCSCAPGWTGFSCQRACDTGHWGPDCSHPCNCSAGHGSCD
AISGLCLCEAGYVGPRCEQSECPQGHFGPGCEQRCQCQHGAACDHVSGACTCPAGWRGTFCEHACPAG
FFGLDCRSACNCTAGAACDAVNGSCLCPAGRRGPRCAETCPAHTYGHNCSQACACFNGASCDPVHGQC
HSGGCLNGGLCDPHTGRCLCPAGWTGDKCQSPAACAKGTFGPHCEGRCACRWGGPCHLATGACLCPPG
WRGPHLSAACLRGWFGEACAQRCSCPPGAACHHVTGACRCPPGFTGSGCEQACPPGSFGEDCAQMCQC
PGENPACHPATGTCSCAAGYHGPSCQQRCPPGRYGPGCEQLCGCLNGGSCDAATGACRCPTGFLGTDC
NLTCPQGRFGPNCTHVCGCGQGAACDPVTGTCLCPPGRAGVRCERGCPQNRFGVGCEHTCSCRNGGLC
HASKRQLLLWPGLDGAALAAGLSPWALRSRLPSGVLLPQQQHV

| | | |
|---|---|---|
| NOV37i, CG56449-07 | SEQ ID NO: 879 | 4783 bp |
| DNA Sequence | ORF Start: ATG at 1 | ORF Stop: TGA at 3595 |

ATGCCCATGGGACATTCTGACAGGTGGTCTTGGCGTCTCCTGAGGCTGGCACTGCCACTCCCAGTCTG
GTTGCCGGCTGGGGGTGGCCGAGGCGCTGACTCTCCATGTCTCTGTTCCAGGCCCCACGTGTGTGCTG
AGCAGGAGCTGACCCTGGTGGGCCGCCGCCAGCCGTGCGTGCAGGCCTTAAGCCACACGGTGCCGGTG
TGGAAGGCCGGCTGTGGGTGGCAGGCGTGGTGCGTGGGTCATGAGCGGAGAACCGTCTACTACATGGG
CTACAGGCAGGTGTATACCACGGAGGCCCGGACCGTGCTCAGGTGCTGCCGAGGGTGGACGCAGCAGC
CCGACGAGGAGGGCTGCCTCTCGGCTGAATGCAGCGCCAGCCTCTGTTTTCACGGTGGCCGTTGTGTG
CCAGGCTCAGCCCAGCCGTGTCACTGTCCCCCCGGCTTCCAGGGACCCCGCTGTCAGTATGATGTGGA
CGAATGCCGAACCCACAACGGTGGCTGCCAGCACCGGTGCGTGAACACCCCAGGCTCCTACCTCTGTG
AGTGCAAGCCCGGCTTCCGGCTCCACACTGACAGCAGGACCTGCCTGGCCATTAACTCCTGCGCCCTG
GGCAATGGCGGCTGCCAGCACCACTGTGTCCAGCTCACAATCACTCGGCATCGCTGCCAGTGCCGGCC
CGGGTTCCAGCTCCAGGAGGACGGCAGGCATTGTGTCCGTAGAAGCCCGTGTGCCAACAGGAACGGCA
GCTGCATGCACAGGTGCCAGGTGGTCCGGGGCCTCGCCCGCTGTGAGTGCCACGTGGGCTATCAGCTA
GCAGCGGACGGCAAGGCCTGTGAAGATGTGGACGAATGTGCCGCAGGGCTGGCCCAGTGTGCCCATGG
CTGCCTCAACACCCAGGGGTCCTTCAAGTGCGTGTGTCACGCGGGCTATGAGCTGGGCGCCGATGGCC
GGCAGTGCTACCGTATTGAGATGGAAATCGTGAACAGCTGTGAGGCCAACAACGGCGGCTGCTCCCAT
GGCTGCAGCCACACCAGTGCTGGGCCCCTGTGCACCTGTCCCCGCGGCTACGAGCTGGACACAGATCA
GAGGACCTGCATCAGATGTCGACGACTGTGCAGACAGCCCGTGCTGCAGCAGGTGTGCACCAACAACC
CTGGCGGGTACGAGTGCGGCTGCTACGCCGGCTACCGGCTCAGTGCCGATGGCTGCGGCTGCGAGGAT
GTGGATGAGTGCGCCTCCAGCCGTGGCGGCTGCGAGCACCACTGCACCAACCTGGCCGGCTCCTTCCA
GTGCTCCTGCGAGGCCGGCTACCGGCTGCACGAGGACCGTAGGGGCTGCAGCGCCCTGGAGGAGCCGA
TGGTGGACCTGGACGGCGAGCTGCCTTTCGTGCGGCCCCTGCCCCACATTGCCGTGCTCCAGGACGAG
CTGCCGCAACTCTTCCAGGATGACGACGTCGGGGCCGATGAGGAAGAGGCAGAGTTGCGGGGCGAACA
CACGCTCACAGAGAAGTTTGTCTGCCTGGATGACTCCTTTGGCCATGACTGCAGCTTGACCTGTGATG
ACTGCAGGAACGGAGGGACCTGCCTCCTGGGCCTGGATGGCTGTGATTGCCCCGAGGGCTGGACTGGG
CTCATCTGCAATGAGAGTTGTCCTCCGGACACCTTTGGGAAGAACTGCAGCTTCTCCTGCAGCTGTCA
GAATGGTGGGACCTGCGACTCTGTCACGGGGGCCTGCCGCTGCCCCCCGGGTGTCAGTGGAACTAACT
GTGAGGATGGCTGCCCCAAGGGCTACTATGGCAAGCACTGTCGCAAGAAATGCAACTGTGCCAACCGG
GGCCGGTGCCACCGCCTCTACGGGGCCTGCCTCTGCGACCCAGGGCTCTACGGCCGCTTCTGCCACCT
CGCCTGCCCGCCGTGGGCCTTTGGGCCGGGCTGCTCGGAGGAGTGCCAGTGTGTGCAGCCCCACACGC
AGTCCTGTGACAAGAGGGATGGCAGCTGCTCCTGCAAGGCTGGCTTCCGGGGCGAGCGCTGTCAGGCA
GAGTGTGAGCTGGGCTACTTTGGGCCGGGGTGCTGGCAGGCATGCACCTGCCCAGTGGGCGTGGCCTG
TGACTCCGTGAGCGGCGAGTGTGGGAAGCGGTGTCCTGCTGGCTTCCAGGGAGAGGACTGTGGCCAAG
AGTGCCCGGTGGGGACGTTTGGCGTGAACTGCTCGAGCTCCTGCTCCTGTGGGGGGGCCCCCTGCCAC
GGGGTCACGGGGCAGTGCCGGTGTCCACCGGGGAGGACTGGGGAAGACTGTGAGGCAGATTGTCCCGA
GGGCCGCTGGGGGCTGGGCTGCCAGGAGATCTGCCCAGCATGCCAGCACGCTGCCCGCTGCGACCCTG
AGACCGGAGCCTGCCTGTGCCTCCCTGGCTTCGTCGGCAGCCGCTGCCAGGACGTGTGCCCAGCAGGC
TGGTATGGTCCCAGCTGCCAGACAAGGTGCTCTTGTGCCAATGATGGGCACTGCCACCCAGCCACCGG
ACACTGCAGCTGTGCCCCCGGGTGGACCGGCTTTAGCTGCCAGAGAGCCTGTGATACTGGGCACTGGG
GACCTGACTGCAGCCACCCCTGCAACTGCAGCGCTGGCCACGGGAGCTGTGATGCCATCAGCGGCCTG
TGTCTGTGTGAGGCTGGCTACGTGGGCCCGCGGTGCGAGCAGCAGTGTCCCCAGGGCCACTTTGGGCC
CGGCTGTGAGCAGCTGTGCCAGTGTCAGCATGGAGCAGCCTGTGACCACGTCAGCGGGGCCTGCACCT
GCCCGGCCGGCTGGAGGGGCACCTTCTGCGAGCATGCCTGCCCGGCCGGCTTCTTTGGATTGGACTGT
CGTAGTGCCTGCAACTGCACCGCCGGAGCTGCCTGTGATGCCGTGAATGGCTCCTGCCTCTGCCCCGC
TGGCCGCCGGGGCCCCCGCTGTGCCGAGACCTGCCCTGCCGGCCTGTACGGCGACAACTGTCGGCATT
CCTGCCTCTGCCAGAACGGAGGGACCTGTGACCCTGTCTCAGGCCACTGTGCGTGCCCAGAGGGCTGG
GCCGGCCTGGCCTGTGAGAAGGAGTGCCCCCCCCGGGACGTCAGAGCTGGCTGCCGGCACAGCGGCGG
TTGCCTCAACGGGGGCCTGTGTGACCCGCACACGGGCCGCTGCCTCTGCCCAGCCGGCTGGACTGGGG
ACAAGTGTCAGAGCCATCCCCACGGGCCCCTCCTAGAAGCCTCCGCAGCCCTCATCTTTCTGCAGCCT
GCCTGCGGGGCTGGTTTGGAGAGGCCTGTGCCCAGCGCTGCAGCTGCCCGCCTGCCGCTGCCTGCCAC
CACGTCACTGGGGCCTGCCGCTGTCCCCCTGGCTTCACTGGCTCCGGCTGCGAGCAGGGATGTCCGCC
CGGGCGGTATGGGCCAGGCTGTGAACAGCTGTGTGGGTGTCTCAACGGGGGCTCCTGTGATGCGGCCA
CGGGGGCCTGCCGCTGCCCCACTGGGTTCCTCGGGACGGACTGCAACCTCACCTGTCCGCAGGGCCGC
TTCGGCCCCAACTGCACCCACGTGTGTGGTTGTGGGCAGGGGGCGGCCTGCGACCCTGTGACCGGCAC
CTGCCTCTGCCCCCCGGGGAGAGCCGGCGTCCGCTGTGAGCGAGGCTGCCCCCAGAACCGGTTTGGCG
TGGGCTGCGAGCACACCTGCTCCTCCAGAAATGGGGGCCTGTGCCACGCCAGCAACGGCAGCTGCTCC
TGTGGCCTGGGCTGGACGGGGCGGCACTGCGAGCTGGCCTGTCCCCCTGGGCGCTACGGAGCCGCCTG
CCATCTGGAGTGCTCCTGCCACAACAACAGCACGGGTGAGCCTGCCACGGGCACCTGCCGCTGCGGCC
CCGGCTTCTATGGCCAGGCCTGCGAGCACCCCTGTCCCCCTGGCTTCCACGGGGCTGGCTGCCAGGGG
TTGTGCTGGTGTCAACATGGAGCCCCCTGCGACCCCATCAGTGGCCGATGCCTCTGCCCTGCCGGCTT
CCACGGCCACTTCTGTGAGAGGGGGTGTGAGCCAGGTTCATTTGGAGAGGGCTGCCACCAGCGCTGTG
ACTGTGACGGGGGGGCACCCTGTGACCCTGTCACCGGTCTCTGCCTTTGCCCACCAGGGCGCTCAGGA
GCCACCTGTAACCTGGATTGCAGAAGGGGCCAGTTTGGGCCCAGCTGCACCCTGCACTGTGACTGCGG
GGGTGGGGCTGACTGCGACCCTGTCAGTGGGCAGTGTCACTGTGTGGATGGCTACATGGGGCCCACGT
GCCGGGAAGGTGGGCCCCTCCGGCTCCCCGAGAACCCGTCCTTAGCCCAGGGCTCAGCGGGCACACTC
CCCGCCTCCAGCAGACCCACATCCCGGAGCGGTGGACCAGCGAGGCACTAGTAGAGGCAGTCCCGTGG
AGCCCGCCTCTCCAGTCCCAGCCAGAGGGGACCCTGGCCTTTGGTGACCACTGAGAAGGACACTTCAC
GGGCCCAGAGCTCCTGGTACTGCCCTTCCTTTGAGGGCCGTGGAGGGCTGTGGACAGCCCAGCAACCT
GTCGCTCTTGGAGGCTGGTGTGGCCTTGAGGAGGGAAGCCTCGCATGGCCGCTGGAAGAGAGGCGCCT
CCTGGCCTGGCTCTGCAGAACCCAGGGGCACGCTCTGGGCCTGGGCTGAGGAAGTCCCGCTCTCCCCG
CGGCTCTGAGTTGGACTGAGGACAGGTGTGGGCGCCAGTGTGGGTGCAGGCGCAGGTGCAGGCACAGC
GCCACTGTCCTCCAGGCAGGCTT

| | | | |
|---|---|---|---|
| NOV371, CG56449-07 | | | |
| Protein Sequence | SEQ ID NO: 880 | 1198 aa | MW at 126170.7kD |

MPMGHSDRWSWRLLRLALPLPVWLPAGGGRGADSPCLCSRPHVCAEQELTLVGRRQPCVQALSHTVPV
WKAGCGWQAWCVGHERRTVYYMGYRQVYTTEARTVLRCCRGWTQQPDEEGCLSAECSASLCFHGGRCV
PGSAQPCHCPPGFQGPRCQYDVDECRTHNGGCQHRCVNTPGSYLCECKPGFRLHTDSRTCLAINSCAL
GNGGCQHHCVQLTITRHRCQCRPGFQLQEDGRHCVRRSPCANPNGSCMHRCQVVRGLARCECHVGYQL
AADGKACEDVDECAAGLAQCAHGCLNTQGSFKCVCHAGYELGADGRQCYRIEMEIVNSCEANNGGCSH
GCSHTSAGPLCTCPRGYELDTDQRTCIRCRRLCRQPVLQQVCTNNPGGYECGCYAGYRLSADGCGCED
VDECASSRGGCEHHCTNLAGSFQCSCEAGYRLHEDRRGCSALEEPMVDLDGELPFVRPLPHIAVLQDE
LPQLFQDDDVGADEEEAELRGEHTLTEKFVCLDDSFGHDCSLTCDDCRNGGTCLLGLDGCDCPEGWTG
LICNESCPPDTFGKNCSFSCSCQNGGTCDSVTGACRCPPGVSGTNCEDGCPKGYYGKHCRKKCNCANR
GRCHRLYGACLCDPGLYGRFCHLACPPWAFGPGCSEECQCVQPHTQSCDKRDGSCSCKAGFRGERCQA
ECELGYFGPGCWQACTCPVGVACDSVSGECGKRCPAGFQGEDCGQECPVGTFGVNCSSSCSCGGAPCH
GVTGQCRCPPGRTGEDCEADCPEGRWGLGCQEICPACQHAARCDPETGACLCLPGFVGSRCQDVCPAG
WYGPSCQTRCSCANDGHCHPATGHCSCAPGWTGFSCQRACDTGHWGPDCSHPCNCSAGHGSCDAISGL
CLCEAGYVGPRCEQQCPQGHFGPGCEQLCQCQHGAACDHVSGACTCPAGWRGTFCEHACPAGFFGLDC
RSACNCTAGAACDAVNGSCLCPAGRRGPRCAETCPAGLYGDNCRHSCLCQNGGTCDPVSGHCACPEGW
AGLACEKECPPRDVRAGCRHSGGCLNGGLCDPHTGRCLCPAGWTGDKCQSHPHGPLLEASAALIFLQP
ACGAGLERPVPSAAAARLPLPATTSLGPAAVPLASLAPAASRDVRPGGMGQAVNSCVGVSTGAPVMRP
RGPAAAPLGSSGRTATSPVRRAASAPTAPTCVVVGRGRPATL

| | | |
|---|---|---|
| NOV37m, CG56449-08 | SEQ ID NO: 881 | 4835 bp |
| DNA Sequence | ORF Start: ATG at 1 | ORF Stop: TAG at 4732 |

ATGTCGTTCCTTGAAGAGGCGAGGGCAGCGGGGCGCGCGGTGGTCCTGGCGTTGGTGCTGCTGCTGCT
CCCCGCCGTGCCCGTGGGCGCCAGCGTTCCGCCGCGGCCCCTGCTCCCGCTGCAGCCCGGCATGCCCC
ACGTGTGTGCTGAGCAGGAGCTGACCCTGGTGGGCCGCCGCCAGCCGTGCGTGCAGGCCTTAAGCCAC
ACGGTGCCGGTGTGGAAGGCCGGCTGTGGGTGGCAGGCGTGGTGCGTGGGTCATGAGCGGAGGACCGT
CTACTACATGGGCTACAGGCAGGTGTATACCACGGAGGCCCGGACCGTGCTCAGGTGCTGCCGAGGGT
GGATGCAGCAGCCCGACGAGGAGGGCTGCCTCTCGGATGTGGGTGAGTGTGCCAACGCCAACGGGGGC
TGTGCGGGTCGGTGCCGGGACACCGTGGGGGGCTTCTACTGCCGCTGGCCCCCCCCCAGCCACCAGCT
GCAGGGTGATGGCGAGACTTGCCAAGATGTGGACGAATGCCGAACCCACAACGGTGGCTGCCAGCACC
GGTGCGTGAACACCCCAGGCTCCTACCTCTGTGAGTGCAAGCCCGGCTTCCGGCTCCACACTGACAGC
AGGACCTGCGCCATTAACTCCTGCGCCCTGGGCAATGGCGGCTGCCAGCACCACTGTGTCCAGCTCAC
AATCACTCGGCATCGCTGCCAGTGCCGGCCCGGGTTCCAGCTCCAGGAGGACGGCAGGCATTGTGTCC
GTAGAAGCCCGTGTGCCAACAGGAACGGCAGCTGCATGCACAGGTGCCAGGTGGTCCGGGGCCTCGCC
CGCTGTGAGTGCCACGTGGGCTATCAGCTAGCAGCGGACGGCAAGGCCTGTGAAGATGTGGACGAATG
TGCCGCAGGGCTGGCCCAGTGTGCCCATGGCTGCCTCAACACCCAGGGGTCCTTCAAGTGCGTGTGTC
ACGCGGGCTATGAGCTGGGCGCCGATGGCCGGCAGTGCTACCGTATTGAGATGGAAATCGTGAACAGC
TGTGAGGCCAACAACGGCGGCTGCTCCCATGGCTGCAGCCACACCAGTGCTGGGCCCCTGTGCACCTG
TCCCCGCGGCTACGAGCTGGACACAGATCAGAGGACCTGCATCAGATGTCGACGACTGTGCAGACAGC
CCGTGCTGCAGCAGGTGTGCACCAACAACCCTGGCGGGTACGAGTGCGGCTGCTACGCCGGCTACCGG
CTCAGTGCCGATGGCTGCGGCTGCGAGGATGTGGATGAGTGCGCCTCCAGCCGTGGCGGCTGCGAGCA
CCACTGCACCAACCTGGCCGGCTCCTTCCAGTGCTCCTGCGAGGCCGGCTACCGGCTGCACGAGGACC
GTAGGGGCTGCAGCGCCCTGGAGGAGCCGATGGTGGACCTGGACGGCGAGCTGCCTTTCGTGCGGCCC
CTGCCCCACATTGCCGTGCTCCAGGACGAGCTGCCGCAACTCTTCCAGGATGACGACGTCGGGGCCGA
TGAGGAAGAGGCAGAGTTGCGGGGCGAACACACGCTCACAGAGAAGTTTGTCTGCCTGGATGACTCCT
TTGGCCATGACTGCAGCTTGACCTGTGATGACTGCAGGAACGGAGGGACCTGCCTCCTGGGCCTGGAT
GGCTGTGATTGCCCCGAGGGCTGGACTGGGCTCATCTGCAATGAGAGTTGTCCTCCGGACACCTTTGG
GAAGAACTGCAGCTTCTCCTGCAGCTGTCAGAATGGTGGGACCTGCGACTCTGTCACGGGGGCCTGCC
GCTGCCCCCCGGGTGTCAGTGGAACTAACTGTGAGGATGGCTGCCCCAAGGGCTACTATGGCAAGCAC
TGTCGCAAGAAATGCAACTGTGCCAACCGGGGCCGGTGCCACCGCCTCTACGGGGCCTGCCTCTGCGA
CCCAGGGCTCTACGGCCGCTTCTGCCACCTCGCCTGCCCGCCGTGGGCCTTTGGGCCGGGCTGCTCGG
AGGAGTGCCAGTGTGTGCAGCCCCACACGCAGTCCTGTGACAAGAGGGATGGCAGCTGCTCCTGCAAG
GCTGGCTTCCGGGGCGAGCGCTGTCAGGCAGAGTGTGAGCCGGGCTACTTTGGGCCGGGGTGCTGGCA
GGCATGCACCTGCCCAGTGGGCGTGGCCTGTGACTCCGTGAGCGGCGAGTGTGGGAAGCGGTGTCCTG
CTGGCTTCCAGGGAGAGGACTGTGGCCAAGAGTGCCCGGTGGGGACCTTTGGCGTGAACTGCTCGAGC
TCCTGCTCCTGTGGGGGGGCCCCCTGCCACGGGGTCACGGGGCAGTGCCGGTGTCCGCCGGGGAGGAC
TGGGGAAGACTGTGAGGCAGGTGAGTGTGAGGGCCTCTGGGGGCTGGGCTGCCAGGAGATCTGCCCAG
CATGCCATAACGCTGCTCGCTGCGACCCTGAGACCGGAGCCTGCCTGTGCCTCCCTGGCTTTGTCGGC
AGCCGCTGCCAGGACTGTGAGGCAGGCTGGTATGGTCCCAGCTGCCAGACAATGTGCTCTTGTGCCAA
TGATGGGCACTGCCACCAAGACACGGGACACTGCAGCTGTGCCCCCGGGTGGACCGGCTTTAGCTGCC
AGAGAGCCTGTGATACTGGGCACTGGGGACCTGACTGCAGCCACCCCTGCAACTGCAGCGCTGGCCAC
GGGAGCTGTGATGCCATCAGCGGCCTGTGTCTGTGTGAGGCTGGCTACGTGGGCCCGCGGTGCGAGCA
GTCAGAGTGTCCCCAGGGCCACTTTGGGCCCGGCTGTGAGCAGCGGTGCCAGTGTCAGCATGGAGCAG
CCTGTGACCACGTCAGCGGGGCCTGCACCTGCCCGGCCGGCTGGAGGGGCACCTTCTGCGAGCATGCC
TGCCCGGCCGGCTTCTTTGGATTGGACTGTCGCAGTGCCTGCAACTGCACCGCCGGAGCTGCCTGTGA
TGCCGTGAATGGCTCCTGCCTCTGCCCCGCTGGCCGCCGGGGCCCCCGCTGTGCCGAGAGTGCCTGCC
CAGCCCACACCTACGGGCACAATTGCAGCCAGGCCTGTGCCTGCTTTAACGGGGCCTCCTGTGACCCT
GTCCACGGGCAGTGCCACTGTGCCCCTGGCTGGATGGGGCCCTCCTGCCTGCAGGCCTGCCCTGCCGG
CCTGTACGGCGACAACTGTCGGCATTCCTGCCTCTGCCAGAACGGAGGGACCTGTGACCCTGTCTCAG
GCCACTGTGCGTGCCCAGAGGGCTGGGCCGGCCTGGCCTGTGAGGTAGAGTGCCTCCCCCGGGACGTC
AGAGCTGGCTGCCGGCACAGCGGCGGTTGCCTCAACGGGGGCCTGTGTGACCCGCACACGGGCCGCTG
CCTCTGCCCAGCCGGCTGGACTGGGGACAAGTGTCAGAGCCCTGCAGCCTGTGCCAAGGGCACATTCG
GGCCTCACTGTGAGGGGCGCTGTGCCTGCCGGTGGGGAGGCCCCTGCCACCTTGCCACCGGGGCCTGC
CTCTGCCCTCCGGGGTGGCGGGGGCCTCATCTTTCTGCAGCCTGCCTGCGGGGCTGGTTTGGAGAGGC
CTGTGCCCAGCGCTGCAGCTGCCCGCCTGGCGCTGCCTGCCACCACGTCACTGGGGCCTGCCGCTGTC
CCCCTGGCTTCACTGGCTCCGGCTGCGAGCAGGCCTGCCCACCCGGCAGCTTTGGGGAGGACTGTGCG
CAGATGTGCCAGTGTCCCGGTGAGAACCCGGCCTGCCACCCTGCCACCGGGACCTGCTCATGTGCTGC
TGGCTACCACGGCCCCAGCTGCCAGCAACGATGTCCGCCCGGGCGGTATGGGCCAGGCTGTGAACAGC
TGTGTGGGTGTCTCAACGGGGGCTCCTGTGATGCGGCCACGGGGGCCTGCCGCTGCCCCACTGGGTTC
CTCGGGACGGACTGCAACCTCACCTGTCCGCAGGGCCGCTTCGGCCCCAACTGCACCCACGTGTGTGG
GTGTGGGCAGGGGGCGGCCTGCGACCCTGTGACCGGCACCTGCCTCTGCCCCCCGGGGAGAGCCGGCG
TCCGCTGTGAGCGAGGCTGCCCCCAGAACCGGTTTGGCGTGGGCTGCGAGCACACCTGCTCCTGCAGA
AATGGGGGCCTGTGCCACGCCAGCAACGGCAGCTGCTCCTGTGGCCTGGGCTGGACGGGGCGGCACTG
CGAGCTGGCCTGTCCCCCTGGGCGCTACGGAGCCGCCTGCCATCTGGAGTGCTCCTGCCACAACAACA
GCACGTGTGAGCCTGCCACGGGCACCTGCCGCTGCGGCCCCGGCTTCTATGGCCAGGCCTGCGAGCAC
CCCTGTCCCCCTGGCTTCCACGGGGCTGGCTGCCAGGGGTTGTGCTGGTGTCAACATGGAGCCCCCTG
CGACCCCATCAGTGGCCGATGCCTCTGCCCTGCCGGCTTCCACGGCCACTTCTGTGAGAGGGGGTGTG
AGCCAGGTTCATTTGGAGAGGGCTGCCACCAGCGCTGTGACTGTGACGGGGGGGCACCCTGTGACCCT
GTCACCGGTCTCTGCCTTTGCCCACCAGGGCGCTCAGGAGCCACCTGTAACCTGGATTGCAGAAGGGG
CCAGTTTGGGCCCAGCTGCACCCTGCACTGTGACTGCGGGGGTGGGGCTGACTGCGACCCTGTCAGTG
GGCAGTGTCACTGTGTGGATGGCTACATGGGGCCCACGTGCCGGGAAGCGGGCACACTGCCCGCCTCC
AGCAGACCCACATCCCGGAGCGGTGGACCAGCGAGGCACTAGTAGAGGCAGTCCCGTGGAGCCCGCCT
CTCCAGTCCCAGCCAGAGGGGACCCTGGCCTTTGGTGACCACTGAGAAGGACACTTCACGGGCCCAGA
GCTCCTG

| | | | |
|---|---|---|---|
| NOV37m, CG56449-08 | | | |
| Protein Sequence | SEQ ID NO: 882 | 1577 aa | MW at 164962.6kD |

MSFLEEARAAGRAVVLALVLLLLPAVPVGASVPPRPLLPLQPGMPHVCAEQELTLVGRRQPCVQALSH
TVPVWKAGCGWQAWCVGHERRTVYYMGYRQVYTTEARTVLRCCRGWMQQPDEEGCLSDVGECANANGG
CAGRCRDTVGGFYCRWPPPSHQLQGDGETCQDVDECRTHNGGCQHRCVNTPGSYLCECKPGFRLHTDS
RTCAINSCALGNGGCQHHCVQLTITRHRCQCRPGFQLQEDGRHCVRRSPCANRNGSCMHRCQVVRGLA
RCECHVGYQLAADGKACEDVDECAAGLAQCAHGCLNTQGSFKCVCHAGYELGADGRQCYRIEMEIVNS
CEANNGGCSHGCSHTSAGPLCTCPRGYELDTDQRTCIRCRRLCRQPVLQQVCTNNPGGYECGCYAGYR
LSADGCGCEDVDECASSRGGCEHHCTNLAGSFQCSCEAGYRLHEDRRGCSALEEPMVDLDGELPFVRP
LPHIAVLQDELPQLFQDDDVGADEEEAELRGEHTLTEKFVCLDDSFGHDCSLTCDDCRNGGTCLLGLD
GCDCPEGWTGLICNESCPPDTFGKNCSFSCSCQNGGTCDSVTGACRCPPGVSGTNCEDGCPKGYYGKH
CRKKCNCANRGRCHRLYGACLCDPGLYGRFCHLACPPWAFGPGCSEECQCVQPHTQSCDKRDGSCSCK
AGFRGERCQAECEPGYFGPGCWQACTCPVGVACDSVSGECGKRCPAGFQGEDCGQECPVGTFGVNCSS
SCSCGGAPCHGVTGQCRCPPGRTGEDCEAGECEGLWGLGCQEICPACHNAARCDPETGACLCLPGFVG
SRCQDCEAGWYGPSCQTMCSCANDGHCHQDTGHCSCAPGWTGFSCQAACDTGHWGPDCSHPCNCSAGH
GSCDAISGLCLCEAGYVGPRCEQSECPQGHFGPGCEQRCQCQHGAACDHVSGACTCPAGWRGTFCEHA
CPAGFFGLDCRSACNCTAGAACDAVNGSCLCPAGRRGPRCAESACPAHTYGHNCSQACACFNGASCDP
JHGQCHCAPGWMGPSCLQACPAGLYGDNCRHSCLCQNGGTCDPVSGHCACPEGWAGLACEVECLPRDV
RAGCRHSGGCLNGGLCDPHTGRCLCPAGWTGDKCQSPAACAKGTFGPHCEGRCACRWGGPCHLATGAC
LCPPGWRGPHLSAACLRGWFGEACAQRCSCPPGAACHHVTGACRCPPGFTGSGCEQACPPGSFGEDCA
QMCQCPGENPACHPATGTCSCAAGYHGPSCQQRCPPGRYGPGCEQLCGCLNGGSCDAATGACRCPTGF
LGTDCNLTCPQGRFGPNCTHVCGCGQGAACDPVTGTCLCPPGRAGVRCERGCPQNRFGVGCEHTCSCR
NGGLCHASNGSCSCGLGWTGRHCELACPPGRYGAACHLECSCHNNSTCEPATGTCRCGPGFYGQACEH
PCPPGFHGAGCQGLCWCQHGAPCDPISGRCLCPAGFHGHFCERGCEPGSFGEGCHQRCDCDGGAPCDP
VTGLCLCPPGRSGATCNLDCRRGQFGPSCTLHCDCGGGADCDPVSGQCHCVDGYMGPTCREAGTLPAS
SRPTSRSGGPARH

| | | |
|---|---|---|
| NOV37n, CG56449-10 | SEQ ID NO: 883 | 5000 bp |
| DNA Sequence | ORF Start: ATG at 169 | ORF Stop: at 4900 |

TGCTGTTACAGGTGGAGGGCAGTGTAGTCTGAGCAGTACTCGTTGCTGCCGCGCGCGCCACCAGACAT
AATAGCTGACAGACTAACAGACTGTTCCTTTCCATGGGTCTTTTCTGCAGTCACCGTCCTTGACACGA
AGCTTTCTAGAAGATCTTCGCGAGGATCCACCATGTCGTTCCTTGAAGAGGCGAGGGCAGCGGGGCGC
GCGGTGGTCCTGGCGTTGGTGCTGCTGCTGCTCCCCGCCGTGCCCGTGGGCGCCAGCGTTCCGCCGCG
GCCCCTGCTCCCGCTGCAGCCCGGCATGCCCCACGTGTGTGCTGAGCAGGAGCTGACCCTGGTGGGCC
GCCGCCAGCCGTGCGTGCAGGCCTTAAGCCACACGGTGCCGGTGTGGAAGGCCGGCTGTGGGTGGCAG
GCGTGGTGCGTGGGTCATGAGCGGAGGACCGTCTACTACATGGGCTACAGGCAGGTGTATACCACGGA
aGGCCCGACCGTGCTCAGGTGCTGCCGAGGGTGGATGCAGCAGCCCGACGAGGAGGGCTGCCTCTCGG
ATGTGGGTGAGTGTGCCAACGCCAACGGGGGCTGTGCGGGTCGGTGCCGGGACACCGTGGGGGGCTTC
TACTGCCGCTGGCCCCCCCCCAGCCACCAGCTGCAGGGTGATGGCGAGACTTGCCAAGATGTGGACGA
ATGCCGAACCCACAACGGTGGCTGCCAGCACCGGTGCGTGAACACCCCAGGCTCCTACCTCTGTGAGT
GCAAGCCCGGCTTCCGGCTCCACACTGACAGCAGGACCTGCGCCATTAACTCCTGCGCCCTGGGCAAT
GGCGGCTGCCAGCACCACTGTGTCCAGCTCACAATCACTCGGCATCGCTGCCAGTGCCGGCCCGGGTT
CCAGCTCCAGGAGGACGGCAGGCATTGTGTCCGTAGAAGCCCGTGTGCCAACAGGAACGGCAGCTGCA
TGCACAGGTGCCAGGTGGTCCGGGGCCTCGCCCGCTGTGAGTGCCACGTGGGCTATCAGCTAGCAGCG
GACGGCAAGGCCTGTGAAGATGTGGACGAATGTGCCGCAGGGCTGGCCCAGTGTGCCCATGGCTGCCT
CAACACCCAGGGGTCCTTCAAGTGCGTGTGTCACGCGGGCTATGAGCTGGGCGCCGATGGCCGGCAGT
GCTACCGTATTGAGATGGAAATCGTGAACAGCTGTGAGGCCAACAACGGCGGCTGCTCCCATGGCTGC
AGCCACACCAGTGCTGGGCCCCTGTGCACCTGTCCCCGCGGCTACGAGCTGGACACAGATCAGAGGAC
CTGCATCAGATGTCGACGACTGTGCAGACAGCCCGTGCTGCAGCAGGTGTGCACCAACAACCCTGGCG
GGTACGAGTGCGGCTGCTACGCCGGCTACCGGCTCAGTGCCGATGGCTGCGGCTGCGAGGATGTGGAT
GAGTGCGCCTCCAGCCGTGGCGGCTGCGAGCACCACTGCACCAACCTGGCCGGCTCCTTCCAGTGCTC
CTGCGAGGCCGGCTACCGGCTGCACGAGGACCGTAGGGGCTGCAGCGCCCTGGAGGAGCCGATGGTGG
ACCTGGACGGCGAGCTGCCTTTCGTGCGGCCCCTGCCCCACATTGCCGTGCTCCAGGACGAGCTGCCG
CAACTCTTCCAGGATGACGACGTCGGGGCCGATGAGGAAGAGGCAGAGTTGCGGGGCGAACACACGCT
CACAGAGAAGTTTGTCTGCCTGGATGACTCCTTTGGCCATGACTGCAGCTTGACCTGTGATGACTGCA
GGAACGGAGGGACCTGCCTCCTGGGCCTGGATGGCTGTGATTGCCCCGAGGGCTGGACTGGGCTCATC
TGCAATGAGAGTTGTCCTCCGGACACCTTTGGGAAGAACTGCAGCTTCTCCTGCAGCTGTCAGAATGG
TGGGACCTGCGACTCTGTCACGGGGGCCTGCCGCTGCCCCCCGGGTGTCAGTGGAACTAACTGTGAGG
ATGGCTGCCCCAAGGGCTACTATGGCAAGCACTGTCGCAAGAAATGCAACTGTGCCAACCGGGGCCGG
TGCCACCGCCTCTACGGGGCCTGCCTCTGCGACCCAGGGCTCTACGGCCGCTTCTGCCACCTCGCCTG
CCCGCCGTGGGCCTTTGGGCCGGGCTGCTCGGAGGAGTGCCAGTGTGTGCAGCCCCACACGCAGTCCT
GTGACAAGAGGGATGGCAGCTGCTCCTGCAAGGCTGGCTTCCGGGGCGAGCGCTGTCAGGCAGAGTGT
GAGCCGGGCTACTTTGGGCCGGGGTGCTGGCAGGCATGCACCTGCCCAGTGGGCGTGGCCTGTGACTC
CGTGAGCGGCGAGTGTGGGAAGCGGTGTCCTGCTGGCTTCCAGGGAGAGGACTGTGGCCAAGAGTGCC
CGGTGGGGACCTTTGGCGTGAACTGCTCGAGCTCCTGCTCCTGTGGGGGGGCCCCCTGCCACGGGGTC
ACGGGGCAGTGCCGGTGTCCGCCGGGGAGGACTGGGGAAGACTGTGAGGCAGGTGAGTGTGAGGGCCT
CTGGGGGCTGGGCTGCCAGGAGATCTGCCCAGCATGCCATAACGCTGCTCGCTGCGACCCTGAGACCG
GAGCCTGCCTGTGCCTCCCTGGCTTTGTCGGCAGCCGCTGCCAGGACTGTGAGGCAGGCTGGTATGGT
CCCAGCTGCCAGACAATGTGCTCTTGTGCCAATGATGGGCACTGCCACCAAGACACGGGACACTGCAG
CTGTGCCCCCGGGTGGACCGGCTTTAGCTGCCAGAGAGCCTGTGATACTGGGCACTGGGGACCTGACT
GCAGCCACCCCTGCAACTGCAGCGCTGGCCACGGGAGCTGTGATGCCATCAGCGGCCTGTGTCTGTGT
GAGGCTGGCTACGTGGGCCCGCGGTGCGAGCAGTCAGAGTGTCCCCAGGGCCACTTTGGGCCCGGCTG
TGAGCAGCGGTGCCAGTGTCAGCATGGAGCAGCCTGTGACCACGTCAGCGGGGCCTGCACCTGCCCGG
CCGGCTGGAGGGGCACCTTCTGCGAGCATGCCTGCCCGGCCGGCTTCTTTGGATTGGACTGTCGCAGT
GCCTGCAACTGCACCGCCGGAGCTGCCTGTGATGCCGTGAATGGCTCCTGCCTCTGCCCCGCTGGCCG
CCGGGGCCCCCGCTGTGCCGAGAGTGCCTGCCCAGCCCACACCTACGGGCACAATTGCAGCCAGGCCT
GTGCCTGCTTTAACGGGGCCTCCTGTGACCCTGTCCACGGGCAGTGCCACTGTGCCCCTGGCTGGATG
GGGCCCTCCTGCCTGCAGGCCTGCCCTGCCGGCCTGTACGGCGACAACTGTCGGCATTCCTGCCTCTG
CCAGAACGGAGGGACCTGTGACCCTGTCTCAGGCCACTGTGCGTGCCCAGAGGGCTGGGCCGGCCTGG
CCTGTGAGGTAGAGTGCCTCCCCCGGGACGTCAGAGCTGGCTGCCGGCACAGCGGCGGTTGCCTCAAC
GGGGGCCTGTGTGACCCGCACACGGGCCGCTGCCTCTGCCCAGCCGGCTGGACTGGGGACAAGTGTCA
GAGCCCTGCAGCCTGTGCCAAGGGCACATTCGGGCCTCACTGTGAGGGGCGCTGTGCCTGCCGGTGGG
GAGGCCCCTGCCACCTTGCCACCGGGGCCTGCCTCTGCCCTCCGGGGTGGCGGGGGCCTCATCTTTCT
GCAGCCTGCCTGCGGGGCTGGTTTGGAGAGGCCTGTGCCCAGCGCTGCAGCTGCCCGCCTGGCGCTGC
CTGCCACCACGTCACTGGGGCCTGCCGCTGTCCCCCTGGCTTCACTGGCTCCGGCTGCGAGCAGGCCT
GCCCACCCGGCAGCTTTGGGGAGGACTGTGCGCAGATGTGCCAGTGTCCCGGTGAGAACCCGGCCTGC
CACCCTGCCACCGGGACCTGCTCATGTGCTGCTGGCTACCACGGCCCCAGCTGCCAGCAACGATGTCC
GCCCGGGCGGTATGGGCCAGGCTGTGAACAGCTGTGTGGGTGTCTCAACGGGGGCTCCTGTGATGCGG
CCACGGGGGCCTGCCGCTGCCCCACTGGGTTCCTCGGGACGGACTGCAACCTCACCTGTCCGCAGGGC
CGCTTCGGCCCCAACTGCACCCACGTGTGTGGGTGTGGGCAGGGGGCGGCCTGCGACCCTGTGACCGG
CACCTGCCTCTGCCCCCCGGGGAGAGCCGGCGTCCGCTGTGAGCGAGGCTGCCCCCAGAACCGGTTTG
GCGTGGGCTGCGAGCACACCTGCTCCTGCAGAAATGGGGGCCTGTGCCACGCCAGCAACGGCAGCTGC
TCCTGTGGCCTGGGCTGGACGGGGCGGCACTGCGAGCTGGCCTGTCCCCCTGGGCGCTACGGAGCCGC
CTGCCATCTGGAGTGCTCCTGCCACAACAACAGCACGTGTGAGCCTGCCACGGGCACCTGCCGCTGCG
GCCCCGGCTTCTATGGCCAGGCCTGCGAGCACCCCTGTCCCCCTGGCTTCCACGGGGCTGGCTGCCAG
GGGTTGTGCTGGTGTCAACATGGAGCCCCCTGCGACCCCATCAGTGGCCGATGCCTCTGCCCTGCCGG
CTTCCACGGCCACTTCTGTGAGAGGGGGTGTGAGCCAGGTTCATTTGGAGAGGGCTGCCACCAGCGCT
GTGACTGTGACGGGGGGGCACCCTGTGACCCTGTCACCGGTCTCTGCCTTTGCCCACCAGGGCGCTCA
GGAGCCACCTGTAACCTGGATTGCAGAAGGGGCCAGTTTGGGCCCAGCTGCACCCTGCACTGTGACTG
CGGGGGTGGGGCTGACTGCGACCCTGTCAGTGGGCAGTGTCACTGTGTGGATGGCTACATGGGGCCCA
CGTGCCGGGAAGCGGGCACACTGCCCGCCTCCAGCAGACCCACATCCCGGAGCGGTGGACCAGCGAGG
CACGTATTCCCCGGGCTCGAGGGTAAGCCTATCCCTAACCCTCTCCTCGGTCTCGATTCTACGCGTAC
CGGTCATCACCACCATCACCATTGAGTTTAATTCAT

| | | | |
|---|---|---|---|
| NOV37n, CG56449-10 | | | |
| Protein Sequence | SEQ ID NO: 884 | 1577 aa | MW at 164962.6kD |

MSFLEEARAAGRAVVLALVLLLLPAVPVGASVPPRPLLPLQPGMPHVCAEQELTLVGRRQPCVQALSH
TVPVWKAGCGWQAWCVGHERRTVYYMGYRQVYTTEARTVLRCCRGWMQQPDEEGCLSDVGECANANGG
CAGRCRDTVGGFYCRWPPPSHQLQGDGETCQDVDECRTHNGGCQHRCVNTPGSYLCECKPGFRLHTDS
RTCAINSCALGNGGCQHHCVQLTITRHRCQCRPGFQLQEDGRHCVRRSPCANRNGSCMHRCQVVRGLA
RCECHVGYQLAADGKACEDVDECAAGTAQCAHGCLNTQGSFKCVCHAGYELGADGRQCYRIEMETVNS
CEANNGGCSHGCSHTSAGPLCTCPRGYELDTDQRTCIRCRRLCRQPVLQQVCTNNPGGYECGCYAGYR
LSADGCGCEDVDECASSRGGCEHHCTNLAGSFQCSCEAGYRLHEDRRGCSALEEPMVDLDGELPFVRP
LPHIAVLQDELPQLFQDDDVGADEEEAELRGEHTLTEKFVCLDDSFGHDCSLTCDDCRIGGTCLLGLD
GCDCPEGWTGLICNESCPPDTFGKNCSFSCSCQNGGTCDSVTGACRCPPGVSGTNCEDGCPKGYYGKH
CRKKCNCANRGRCHRLYGACLCDPGLYGRFCHLACPPWAFGPGCSEECQCVQPHTQSCDKRDGSCSCK
AGFRGERCQAECEPGYFGPGCWQACTCPVGVACDSVSGECGKRCPAGFQGEDCGQECPVGTFGVNCSS
SCSCGGAPCHGVTGQCRCPPGRTGEDCEAGECEGLWGLGCQEICPACHNAARCDPETGACLCLPGFVG
GSCDAISGLCLCEAGYVGPRCEQSECPQGHFGPGCEQRCQCQHGAACDHVSGACTCPAGWRGTFCEHA
CPAGFFGLDCRSACNCTAGAACDAVNGSCLCPAGRRGPRCAESACPAHTYGHNCSQACACFNGASCDP
VHGQCHCAPGWMGPSCLQACPAGLYGDNCRHSCLCQNGGTCDPVSGHCACPEGWAGLACEVECLPRDV
RAGCRHSGGCLNGGLCDPHTGRCLCPAGWTGDKCQSPAACAKGTFGPHCEGRCACRWGGPCHLATGAC
LCPPGWRGPHLSAACLRGWFGEACAQRCSCPPGAACHHVTGACRCPPGFTGSGCEQACPPGSFGEDCA
QMCQCPGENPACHPATGTCSCAAGYHGPSCQQRCPPGRYGPGCEQLCGCLNGGSCDAATGACRCPTGF
LGTDCNLTCPQGRFGPNCTHVCGCGQGAACDPVTGTCLCPPGPAGVRCERGCPQNRFGVGCEHTCSCR
NGGLCHASNGSCSCGLGWTGRHCELACPPGRYGAACHLECSCHNNSTCEPATGTCRCGPGFYGQACEH
PCPPGFHGAGCQGLCWCQHGAPCDPISGRCLCPAGFHGHFCERGCEPGSFGEGCHQRCDCDGGAPCDP
VTGLCLCPPGRSGATCNLDCRRGQFGPSCTLHCDCGGGADCDPVSGQCHCVDGYMGPTCREAGTLPAS
SRPTSRSGGPARH

| | | |
|---|---|---|
| NOV37o, CG56449-1 | SEQ ID NO: 885 | 5005 bp |
| DNA Sequence | ORF Start: at 258 | ORF Stop: at 4899 |

AACGGGTGGAGGGCAGTGTAGTCTGAGCAGTACTCGTTGCTGCCGCGCGCGCCACCAGACATAATAGC
TGACAGACTAACAGACTGTTCCTTTCCATGGGTCTTTTCTGCAGTCACCGTCCTTGACACGAAGCTCT
AGCCACCATGGAGACAGACACACTCCTGCTATGGGTACTGCTGCTCTGGGTTCCAGGTTCCACTGGTG
ACGCGGCCCAGCCGGCCAGGCGCGCGCGCCGTACGAAGCTTTCGCGAGGATCCAGCGTTCCGCCGCGG
CCCCTGCTCCCGCTGCAGCCCGGCATGCCCCACGTGTGTGCTGAGCAGGAGCTGACCCTGGTGGGCCG
CCGCCAGCCGTGCGTGCAGGCCTTAAGCCACACGGTGCCGGTGTGGAAGGCCGGCTGTGGGTGGCAGG
CGTGGTGCGTGGGTCATGAGCGGAGGACCGTCTACTACATGGGCTACAGGCAGGTGTATACCACGGAG
GCCCGGACCGTGCTCAGGTGCTGCCGAGGGTGGATGCAGCAGCCCGACGAGGAGGGCTGCCTCTCGGA
TGTGGGTGAGTGTGCCAACGCCAACGGGGGCTGTGCGGGTCGGTGCCGGGACACCGTGGGGGGCTTCT
ACTGCCGCTGGCCCCCCCCCAGCCACCAGCTGCAGGGTGATGGCGAGACTTGCCAAGATGTGGACGAA
TGCCGAACCCACAACGGTGGCTGCCAGCACCGGTGCGTGAACACCCCAGGCTCCTACCTCTGTGAGTG
CAAGCCCGGCTTCCGGCTCCACACTGACAGCAGGACCTGCGCCATTAACTCCTGCGCCCTGGGCAATG
GCGGCTGCCAGCACCACTGTGTCCAGCTCACAATCACTCGGCATCGCTGCCAGTGCCGGCCCGGGTTC
CAGCTCCAGGAGGACGGCAGGCATTGTGTCCGTAGAAGCCCGTGTGCCAACAGGAACGGCAGCTGCAT
GCACAGGTGCCAGGTGGTCCGGGGCCTCGCCCGCTGTGAGTGCCACGTGGGCTATCAGCTAGCAGCGG
ACGGCAAGGCCTGTGAAGATGTGGACGAATGTGCCGCAGGGCTGGCCCAGTGTGCCCATGGCTGCCTC
AACACCCAGGGGTCCTTCAAGTGCGTGTGTCACGCGGGCTATGAGCTGGGCGCCGATGGCCGGCAGTG
CTACCGTATTGAGATGGAAATCGTGAACAGCTGTGAGGCCAACAACGGCGGCTGCTCCCATGGCTGCA
GCCACACCAGTGCTGGGCCCCTGTGCACCTGTCCCCGCGGCTACGAGCTGGACACAGATCAGAGGACC
TGCATCAGATGTCGACGACTGTGCAGACAGCCCGTGCTGCAGCAGGTGTGCACCAACAACCCTGGCGG
GTACGAGTGCGGCTGCTACGCCGGCTACCGGCTCAGTGCCGATGGCTGCGGCTGCGAGGATGTGGATG
AGTGCGCCTCCAGCCGTGGCGGCTGCGAGCACCACTGCACCAACCTGGCCGGCTCCTTCCAGTGCTCC
TGCGAGGCCGGCTACCGGCTGCACGAGGACCGTAGGGGCTGCAGCGCCCTGGAGGAGCCGATGGTGGA
CCTGGACGGCGAGCTGCCTTTCGTGCGGCCCCTGCCCCACATTGCCGTGCTCCAGGACGAGCTGCCGC
AACTCTTCCAGGATGACGACGTCGGGGCCGATGAGGAAGAGGCAGAGTTGCGGGGCGAACACACGCTC
GAACGGAGGGACCTGCCTCCTGGGCCTGGATGGCTGTGATTGCCCCGAGGGCTGGACTGGGCTCATCT
AGCATGAGAGTTGTCCTCCGGACACCTTTGGGAAGAACTGCAGCTTCTCCTGCAGCTGTCAGAATGGT
GGGACCTGCGACTCTGTCACGGGGGCCTGCCGCTGCCCCCCGGGTGTCAGTGGAACTAACTGTGAGGA
TGGCTGCCCCAAGGGCTACTATGGCAAGCACTGTCGCAAGAAATGCAACTGTGCCAACCGGGGCCGGT
GCCACCGCCTCTACGGGGCCTGCCTCTGCGACCCAGGGCTCTACGGCCGCTTCTGCCACCTCGCCTGC
CCGCCGTGGGCCTTTGGGCCGGGCTGCTCGGAGGAGTGCCAGTGTGTGCAGCCCCACACGCAGTCCTG
TGACAAGAGGGATGGCAGCTGCTCCTGCAAGGCTGGCTTCCGGGGCGAGCGCTGTCAGGCAGAGTGTG
AGCCGGGCTACTTTGGGCCGGGGTGCTGGCAGGCATGCACCTGCCCAGTGGGCGTGGCCTGTGACTCC
GTGAGCGGCGAGTGTGGGAAGCGGTGTCCTGCTGGCTTCCAGGGAGAGGACTGTGGCCAAGAGTGCCC
GGTGGGGACCTTTGGCGTGAACTGCTCGAGCTCCTGCTCCTGTGGGGGGGCCCCCTGCCACGGGGTCA
CGGGGCAGTGCCGGTGTCCGCCGGGGAGGACTGGGGAAGACTGTGAGGCAGGTGAGTGTGAGGGCCTC
TGGGGGCTGGGCTGCCAGGAGATCTGCCCAGCATGCCATAACGCTGCTCGCTGCGACCCTGAGACCGG
AGCCTGCCTGTGCCTCCCTGGCTTTGTCGGCAGCCGCTGCCAGGACTGTGAGGCAGGCTGGTATGGTC
CCAGCTGCCAGACAATGTGCTCTTGTGCCAATGATGGGCACTGCCACCAAGACACGGGACACTGCAGC
TGTGCCCCCGGGTGGACCGGCTTTAGCTGCCAGAGAGCCTGTGATACTGGGCACTGGGGACCTGACTG
CAGCCACCCCTGCAACTGCAGCGCTGGCCACGGGAGCTGTGATGCCATCAGCGGCCTGTGTCTGTGTG
AGGCTGGCTACGTGGGCCCGCGGTGCGAGCAGTCAGAGTGTCCCCAGGGCCACTTTGGGCCCGGCTGT
GAGCAGCGGTGCCAGTGTCAGCATGGAGCAGCCTGTGACCACGTCAGCGGGGCCTGCACCTGCCCGGC
CGGCTGGAGGGGCACCTTCTGCGAGCATGCCTGCCCGGCCGGCTTCTTTGGATTGGACTGTCGCAGTG
CCTGCAACTGCACCGCCGGAGCTGCCTGTGATGCCGTGAATGGCTCCTGCCTCTGCCCCGCTGGCCGC
CGGGGCCCCCGCTGTGCCGAGAGTGCCTGCCCAGCCCACACCTACGGGCACAATTGCAGCCAGGCCTG
TGCCTGCTTTAACGGGGCCTCCTGTGACCCTGTCCACGGGCAGTGCCACTGTGCCCCTGGCTGGATGG
GGCCCTCCTGCCTGCAGGCCTGCCCTGCCGGCCTGTACGGCGACAACTGTCGGCATTCCTGCCTCTGC
CTGTGAGGTAGAGTGCCTCCCCCGGGACGTCAGAGCTGGCTGCCGGCACAGCGGCGGTTGCCTCAACG
GGGGCCTGTGTGACCCGCACACGGGCCGCTGCCTCTGCCCAGCCGGCTGGACTGGGGACAAGTGTCAG
AGCCCTGCAGCCTGTGCCAAGGGCACATTCGGGCCTCACTGTGAGGGGCGCTGTGCCTGCCGGTGGGG
AGGCCCCTGCCACCTTGCCACCGGGGCCTGCCTCTGCCCTCCGGGGTGGCGGGGGCCTCATCTTTCTG
CAGCCTGCCTGCGGGGCTGGTTTGGAGAGGCCTGTGCCCAGCGCTGCAGCTGCCCGCCTGGCGCTGCC
TGCCACCACGTCACTGGGGCCTGCCGCTGTCCCCCTGGCTTCACTGGCTCCGGCTGCGAGCAGGCCTG
CCCACCCGGCAGCTTTGGGGAGGACTGTGCGCAGATGTGCCAGTGTCCCGGTGAGAACCCGGCCTGCC
ACCCTGCCACCGGGACCTGCTCATGTGCTGCTGGCTACCACGGCCCCAGCTGCCAGCAACGATGTCCG
CCCGGGCGGTATGGGCCAGGCTGTGAACAGCTGTGTGGGTGTCTCAACGGGGGCTCCTGTGATGCGGC
CACGGGGGCCTGCCGCTGCCCCACTGGGTTCCTCGGGACGGACTGCAACCTCACCTGTCCGCAGGGCC
GCTTCGGCCCCAACTGCACCCACGTGTGTGGGTGTGGGCAGGGGGCGGCCTGCGACCCTGTGACCGGC
ACCTGCCTCTGCCCCCCGGGGAGAGCCGGCGTCCGCTGTGAGCGAGGCTGCCCCCAGAACCGGTTTGG
CGTGGGCTGCGAGCACACCTGCTCCTGCAGAAATGGGGGCCTGTGCCACGCCAGCAACGGCAGCTGCT
CCTGTGGCCTGGGCTGGACGGGGCGGCACTGCGAGCTGGCCTGTCCCCCTGGGCGCTACGGAGCCGCC
TGCCATCTGGAGTGCTCCTGCCACAACAACAGCACGTGTGAGCCTGCCACGGGCACCTGCCGCTGCGG
CCCCGGCTTCTATGGCCAGGCCTGCGAGCACCCCTGTCCCCCTGGCTTCCACGGGGCTGGCTGCCAGG
GGTTGTGCTGGTGTCAACATGGAGCCCCCTGCGACCCCATCAGTGGCCGATGCCTCTGCCCTGCCGGC
TTCCACGGCCACTTCTGTGAGAGGGGGTGTGAGCCAGGTTCATTTGGAGAGGGCTGCCACCAGCGCTG
TGACTGTGACGGGGGGGCACCCTGTGACCCTGTCACCGGTCTCTGCCTTTGCCCACCAGGGCGCTCAG
GGGGGTGGGGCTGACTGCGACCCTGTCAGTGGGCAGTGTCACTGTGTGGATGGCTACATGGGGCCCAC
GTGCCGGGAAGCGGGCACACTGCCCGCCTCCAGCAGACCCACATCCCGGAGCGGTGGACCAGCGAGGC
ACGAATTCCCCGGGCTCGAGGGTAAGCCTATCCCTAACCCTCTCCTCGGTCTCGATTCTACGCGTACC
GGTCATCACCACCATCACCATTGAGTTTAATTCATTGATTT

| | | | |
|---|---|---|---|
| NOV37o, CG56449-11 | | | |
| Protein Sequence | SEQ ID NO: 886 | 1547 aa | MW at 161933.0kD |

SVPPRPLLPLQPGMPHVCAEQELTLVGRRQPCVQALSHTVPVWKAGCGWQAWCVGHERRTVYYMGYRQ
VYTTEARTVLRCCRGWMQQPDEEGCLSDVGECANANGGCAGRCRDTVGGFYCRWPPPSHQLQGDGETC
QDVDECRTHNGGCQHRCVNTPGSYLCECKPGFRLHTDSRTCAINSCALGNGGCQHHCVQLTITRHRCQ
CRPGFQLQEDGRHCVRRSPCANRNGSCMHRCQVVRGLARCECHVGYQLAADGKACEDVDECAAGLAQC
AHGCLNTQGSFKCVCHAGYELGADGRQCYRIEMEIVNSCEANNGGCSHGCSHTSAGPLCTCPRGYELD
TDQRTCIRCRRLCRQPVLQQVCTNNPGGYECGCYAGYRLSADGCGCEDVDECASSRGGCEHHCTNLAG
SFQCSCEAGYRLHEDRRGCSALEEPMVDLDGELPFVRPLPHIAVLQDELPQLFQDDDVGADEEEAELR
GEHTLTEKFVCLDDSFGHDCSLTCDDCRNGGTCLLGLDGCDCPEGWTGLICNESCPPDTFGKNCSFSC
SCQNGGTCDSVTGACRCPPGVSGTNCEDGCPKGYYGKHCRKKCNCANRGRCHRLYGACLCDPGLYGRF
CHLACPPWAFGPGCSEECQCVQPHTQSCDKRDGSCSCKAGFRGERCQAECEPGYFGPGCWQACTCPVG
VACDSVSGECGKRCPAGFQGEDCGQECPVGTFGVNCSSSCSCGGAPCHGVTGQCRCPPGRTGEDCEAG
ECEGLWGLGCQEICPACHNAARCDPETGACLCLPGFVGSRCQDCEAGWYGPSCQTMCSCANDGHCHQD
TGHCSCAPGWTGFSCQHACDTGHWGPDCSHPCNCSAGHGSCDAISGLCLCEAGYVGPRCEQSECPQGH
FGPGCEQRCQCQHGAACDHVSGACTCPAGWRGTFCEHACPAGFFGLDCRSACNCTAGAACDAVNGSCL
CPAGRRGPRCAESACPAHTYGHNCSQACACFNGASCDPVHGQCHCAPGWMGPSCLQACPAGLYGDNCR
HSCLCQNGGTCDPVSGHCACPEGWAGLACEVECLPRDVRAGCRHSGGCLNGGLCDPHTGRCLCPAGWT
GDKCQSPAACAKGTFGPHCEGRCACRWGGPCHLATGACLCPPGWRGPHLSAACLRGWFGEACAQRCSC
PPGAACHHVTGACRCPPGFTGSGCEQACPPGSFGEDCAQMCQCPGENPACHPATGTCSCAAGYHGPSC
QQRCPPGRYGPGCEQLCGCLNGGSCDAATGACRCPTGFLGTDCNLTCPQGRFGPNCTHVCGCGQGAAC
DPVTGTCLCPPGRAGVRCERGCPQNRFGVGCEHTCSCRNGGLCHASNGSCSCGLGWTGRHCELACPPG
RYGAACHLECSCHNNSTCEPATGTCRCGPGFYGQACEHPCPPGFHGAGCQGLCWCQHGAPCDPISGRC
LCPAGFHGHFCERGCEPGSFGEGCHQRCDCDGGAPCDPVTGLCLCPPGRSGATCNLDCRRGQFGPSCT
LHCDCGGGADCDPVSGQCHCVDGYMGPTCREAGTLPASSRPTSRSGGPARH

| | | |
|---|---|---|
| NOV37p, | | |
| SNP13382514 of | SEQ ID NO: 887 | 877 bp |
| CG56449-04, | ORF Start: ATG at 25 | ORF Stop: TAG at 535 |
| DNA Sequence | SNP Pos: 430 | SNP Change: G to A |

CCGGAGCTGCCTGTGATGCCGTGAATGGCTCCTGCCTCTGCCCCGCTGGCCGCCGGGGCCCCCGCTGT
GCCGAGACCTGCCCTGCCGGCCTGTACGGCGACAACTGTCGGCATTCCTGCCTCTGCCAGAACGGAGG
GACCTGTGACCCTGTCTCAGGCCTGCGAGCACCCCTGTCCCCCTGGCTTCCACGGGGCTGGCCGCCAG
GGGTTGTGCTGGTGTCAACATGGAGCCCCCTGCGACCCCATCAGTGGCCGATGCCTCTGCCCTGCCGG
CTTCCACGGCCACTTCTGTGAGAGGGATTGCAGAAGGGGCCAGTTTGGGCCCAGCTGCACCCTGCACT
GTGACTGCGGGGGTGGGGCTGACTGCGACCCTGTCAGTGGGCAGTGTCACTGTGTGGATGGCTACATG
GGGCCCACGTGCCGGGAAGGTAGGCCCCTCCGGCTCCCCGAGAACCCGTCCTTAGCCCAGGGCTCAGC
GGGCACACTGCCCGCCTCCAGCAGACCCACATCCCGGAGCGGTGGACCAGCGAGGCACTAGTAGAGGC
AGTCCCGTGGAGCCCGCCTCTCCAGTCCCAGCCAGAGGGGACCCTGGCCTTTGGTGACCACTGAGAAG
GACACTTCACGGGCCCAGAGCTCCTGGTACTGCCCTTCCTTTGAGGGCCGTGGAGGGCTGTGGACAGC
CCAGCAACCTGTCGCTCTTGGAGGCTGGTGTGGCCTTGAGGAGGGAAGCCTCGCATGGCCGCTGGAAG
AGAGGCGTCTCCTGGCCTGGCTCTGCAGAACCCAGGGGCACGCTCTGGGCCTGGGCTGAGGAAGTCCC
GCTCTCCCGCGGCTCTGAGTTGGACTGAGGACAGGTGTGGGCGCCAGTGTGGGTGCAGGCG

| | | | |
|---|---|---|---|
| NOV37p, | | | |
| SNP13382514 of | | | |
| CG56449-04, | SEQ ID NO: 888 | | MW at 17222.2kD |
| Protein Sequence | SNP Pos: 136 | 170 aa | SNP Change: Gly to Arg |

MAPASAPLAAGAPAVPRPALPACTATTVGIPASARTEGPVTLSQACEHPCPPGFHGAGRQGLCWCQHG
APCDPISGRCLCPAGFHGHFCERDCRRGQFGPSCTLHCDCGGGADCDPVSGQCHCVDGYMGPTCREGR
PLRLPENPSLAQGSAGTLPASSRPTSRSGGPARH

A ClustalW comparison of the above protein sequences yields the following sequence alignment shown in Table 37B.

TABLE 37B

Comparison of the NOV37 protein sequences.

| | |
|---|---|
| NOV37a | ------------------------------------------------------------ |
| NOV37b | -----------------------------------------------MSFLEEARAAGRA |
| NOV37c | ------------------------------------------------------------ |
| NOV37d | -------------------------------------------TGSTMSFLEEARAAGRA |
| NOV37e | SEQYSLLPRAPPDIIADRLTDCSFPWVFSAVTVLDTKLSRRSSRGSTMSFLEEAPAAGRA |
| NOV37f | --------------------------------------------HEALATMETDTLLLWV |
| NOV37g | -----------------------------------------------MPMGHSDRWSWRL |
| NOV37h | -----------------------------------------------MPMGHSDRWSWRL |
| NOV37i | -----------------------------------------------MPMGHSDRWSWRL |
| NOV37j | ------------------------------------------------------------ |
| NOV37k | -----------------------------------------------MPMGHSDRWSWRL |
| NOV37l | -----------------------------------------------MPMGHSDRWSWRL |
| NOV37m | -----------------------------------------------MSFLEEARAAGRA |
| NOV37n | -----------------------------------------------MSFLEEARAAGPA |
| NOV37o | ------------------------------------------------------------ |
| NOV37a | ------------------------------------------------------------ |
| NOV37b | VVLALVLLLLPAVPVGASVPPRPLLPLQPG-----------MPHVCAEQELTLVGRRQPC |
| NOV37c | ------------------------------------------------------------ |
| NOV37d | VVLALVLLLLPAVPVGASVPPRPLLPLQPG-----------MPHVCAEQELTLVGRRQPC |
| NOV37e | VVLALVLLLLPAVPVGASVPPRPLLPLQPG-----------MPHVCAEQELTLVGRRQPC |
| NOV37f | LLLWVPGSTGDAAQPARRARRTKLSRGSSVPPRPLLPLQPGMPHVCAEQELTLVGRRQPC |
| NOV37g | LRLALPLPVWLPAGGGRGADSPCLCS---------------RPHVCAEQELTLVGRRQPC |
| NOV37h | LRLALPLPVWLPAGGGRGADSPCLCS---------------RPHVCAEQELTLVGRRQPC |
| NOV37i | LRLALPLPVWLPAGGGRGADSPCLCS---------------RPHVCAEQELTLVGRRQPC |
| NOV37j | ------------------------------------------------------------ |
| NOV37k | LRLALPLPVWLPAGGGRGADSPCLCS---------------RPHVCAEQELTLVGRRQPC |
| NOV37l | LRLALPLPVWLPAGGGRGADSPCLCS---------------RPHVCAEQELTLVGRRQPC |
| NOV37m | VVLALVLLLLPAVPVGASVPPRPLLPLQPG-----------MPHVCAEQELTLVGRRQPC |
| NOV37n | VVLALVLLLLPAVPVGASVPPRPLLPLQPG-----------MPHVCAEQELTLVGRRQPC |
| NOV37o | ---SVPPRPLLPLQPG-------------------------MPHVCAEQELTLVGRRQPC |
| NOV37a | ------------------------------------------------------------ |
| NOV37b | VQALSHTVPVWRAGCGWQAWCVGHERRTVYYMGYRQVYTTEARTVLRCCRGWTQQPDEEG |
| NOV37c | ------------------------------------------------------------ |
| NOV37d | VQALSHTVPVWKAGCGWQAWCVGHERRTVYYMGYRQVYTTEARTVLRCCRGWTQQPDEEG |
| NOV37e | VQALSHTVPVWKAGCGWQAWCVGHERRTVYYMGYRQVYTTEARTVLRCCRGWMQQPDEEG |
| NOV37f | VQALSHTVPVWKAGCGWQAWCVGHERRTVYYMGYRQVYTTEARTVLRCCRGWMQQPDEEG |
| NOV37g | VQALSHTVPVWKAGCGWQAWCVGHERRTVYYMGYRQVYTTEARTVLRCCRGWMQQPDEEG |
| NOV37h | VQALSHTVPVWKAGCGWQAWCVGHERRTVYYMGYRQVYTTEARTVLRCCRGWTQQPDEEG |
| NOV37i | VQALSHTVPVWKAGCGWQAWCVGHERRTVYYMGYRQVYTTEARTVLRCCRGWTQQPDEEG |
| NOV37j | ------------------------------------------------------------ |
| NOV37k | VQALSHTVPVWKAGCGWQAWCVGHERRTVYYMGYRQVYTTEARTVLRCCRGWMQQPDEEG |
| NOV37l | VQALSHTVPVWKAGCGWQAWCVGHERRTVYYMGYRQVYTTEARTVLRCCRGWTQQPDEEG |
| NOV37ni VQALSHTVPVWKAGCGWQAWCVGHERRTVYYMGYRQVYTTEARTVLRCCRGWMQQPDEEG | |
| NOV37n | VQALSHTVPVWKAGCGWQAWCVGHERRTVYYMGYRQVYTTEARTVLRCCRGWMQQPDEEG |
| NOV37o | VQALSHTVPVWKAGCGWQAWCVGHERRTVYYMGYRQVYTTEARTVLRCCRGWMQQPDEEG |
| NOV37a | ------------------------------------------------------------ |
| NOV37b | CLS--AECSASLCFHGGRCVPGS-AQPCHCPP---GFQGP-RCQYDVDECRTHNGGCQHR |
| NOV37c | ------------------------------------------------------------ |
| NOV37d | CLS--AECSAGLCFHGGRCVPGS-AQPCHCPPG---FQGP-RCQYDVDECRTHNGGCQHR |
| NOV37e | CLSDVGECANANGGCAGRCRDTVGGFYCRWPPPSHQLQGDGETCQDVDECRTHNGGCQHR |
| NOV37f | CLSDVGECANANGGCAGRCRDTVGGFYCRWPPPSHQLQGDGETCQDVDECRTHNGGCQHR |
| NOV37g | CLSDVGECANANGGCAGRCRDTVGGFYCRWPPPSHQLQGDGETCQDVDECRTHNGGCQHR |
| NOV37h | CLS--AECSASLCFHGGRCVPGS-AQPCHCPP-G--FQGP-RCQYDVDECRTHNGGCQHR |
| NOV37i | CLS--AECSASLCFHGGRCVPGS-AQPCHCPPG---FQGP-RCQYDVDECRTHNGGCQHR |
| NOV37j | ------------------------------------------------------------ |
| NOV37k | CLSDVGECANANGGCAGRCRDTVGGFYCRWPPPSHQLQGDGETCQDVDECRTHNGGCQHR |
| NOV37l | CLS--AECSASLCFHGGRCVPGS-AQPCHCPP-G--FQGP-RCQYDVDECRTHNGGCQHR |
| NOV37m | CLSDVGECANANGGCAGRCRDTVGGFYCRWPPPSHQLQGDGETCQDVDECRTHNGGCQHR |
| NOV37n | CLSDVGECANANGGCAGRCRDTVGGFYCRWPPPSHQLQGDGETCQDVDECRTHNGGCQHR |
| NOV37o | CLSDVGECANANGGCAGRCRDTVGGFYCRWPPPSHQLQGDGETCQDVDECRTHNGGCQHR |
| NOV37a | ------------------------------------------------------------ |
| NOV37b | CVNTPGSYLCECKPGFRLHTDSRTCLAINSCALGNGGCQHHCVQLTITRHRCQCRPGFQL |
| NOV37c | ------------------------------------------------------------ |
| NOV37d | CVNTPGSYLCECKPGFRLHTDSRTCLAINSCALGNGGCQHHCVQLTITRHRCQCRPGFQL |
| NOV37e | CVNTPGSYLCECKPGFRLHTDSRTC-AINSCALGNGGCQHHCVQLTITRHRCQCRPGFQL |
| NOV37f | CVNTPGSYLCECKPGFRLHTDSRTC-AINSCALGNGGCQHHCVQLTITRHRCQCRPGFQL |
| NOV37g | CVNTPGSYLCECKPGFRLHTDSRTC-ATNSCALGNGGCQHHCVQLTITRHRCQCRPGFQL |
| NOV37h | CVNTPGSYLCECKPGFRLHTDSRTCLAINSCALGNGGCQHHCVQLTITRHRCQCRPGFQL |
| NOV37i | CVNTPGSYLCECKPGFRLHTDSRTCLAINSCALGNGGCQHHCVQLTITRHRCQCRPGFQL |
| NOV37j | ------------------------------------------------------------ |
| NOV37k | CVNTPGSYLCECKPGFRLHTDSRTC-AINSCALGNGGCQHHCVQLTITRHRCQCRPGFQL |
| NOV37l | CVNTPGSYLCECKPGFRLHTDSRTCLAINSCALGNGGCQHHCVQLTITRHRCQCRPGFQL |
| NOV37m | CVNTPGSYLCECKPGFRLHTDSRTC-AINSCALGNGGCQHHCVQLTITRHRCQCRPGFQL |
| NOV37n | CVNTPGSYLCECKPGFRLHTDSRTC-AINSCALGNGGCQHHCVQLTITRHRCQCRPGFQL |
| NOV37o | CVNTPGSYLCECKPGFRLHTDSRTC-AINSCALGNGGCQHHCVQLTITRHRCQCRPGFQL |
| NOV37a | ------------------------------------------------------------ |
| NOV37b | QEDGRHCVRRSPCANRNGSCMHRCQVVRGLARCECHVGYQLAADGKACEDVDECAAGLAQ |
| NOV37c | ------------------------------------------------------------ |
| NOV37d | QEDGRHCVRRSPCANRNGSCMHRCQVVRGLARCECHVGYQLAADGKACEDVDECAAGLAQ |
| NOV37e | QEDGRHCVRRSPCANRNGSCMHRCQVVRGLARCECHVGYQLAADGKACEDVDECAAGLAQ |
| NOV37f | QEDGRHCVRRSPCANRNGSCMHRCQVVRGLARCECHVGYQLAADGKACEDVDECAAGLAQ |
| NOV37g | QEDGRHCVRRSPCANRNGSCMHRCQVVRGLARCECHVGYQLAADGKACEDVDECAAGIAQ |
| NOV37h | QEDGRHCVRRSPCANRNGSCMHRCQVVRGLARCECHVGYQLAADGKACEDVDECAAGLAQ |
| NOV37i | QEDGRHCVRRSPCANRNGSCMHRCQVVRGLARCECHVGYQLAADGKACEDVDECAAGLAQ |
| NOV37j | ------------------------------------------------------------ |
| NOV37k | QEDGRHCVRRSPCANRNGSCMHRCQVVRGLARCECHVGYQLAADGKACEDVDECAAGLAQ |
| NOV37l | QEDGRHCVRRSPCANRNGSCMHRCQVVRGLARCECHVGYQLAADGKACEDVDECAAGLAQ |
| NOV37m | QEDGRHCVRRSPCANRNGSCMHRCQVVRGLARCECHVGYQLAADGKACEDVDECAAGLAQ |
| NOV37n | QEDGRHCVRRSPCANRNGSCMHRCQVVRGLARCECHVGYQLAADGKACEDVDECAAGLAQ |
| NOV37o | QEDGRHCVRRSPCANRNGSCMHRCQVVRGLARCECHVGYQLAADGKACEDVDECAAGLAQ |
| NOV37a | ------------------------------------------------------------ |
| NOV37b | CAHGCLNTQGSFKCVCHAGYELGADGRQCYRIEMEIVNSCEANNGGCSHGCSHTSAGPLC |
| NOV37c | ------------------------------------------------------------ |
| NOV37d | CAHGCLNTQGSFKCVCHAGYELGADGRQCYRIEMEIVNSCEANNGGCSHGCSHTSAGPLC |
| NOV37e | CAHGCLNTQGSFKCVCHAGYELGADGRQCYRIEMEIVNSCEANNGGCSHGCSHTSAGPLC |
| NOV37i | CAHGCLNTQGSFKCVCHAGYELGADGRQCYRIEMEIVNSCEANNGGCSHGCSHTSAGPLC |
| NOV37g | CAHGCLNTQGSFKCVCHAGYELGADGRQCYRIEMEIVNSCEANNGGCSHGCSHTSAGPLC |
| NOV37h | CAHGCLNTQGSFKCVCHAGYELGADGRQCYRIEMEIVNSCEANNGGCSHGCSHTSAGPLC |
| NOV37i | CAHGCLNTQGSFKCVCHAGYELGADGRQCYRIEMEIVNSCEANNGGCSHGCSHTSAGPLC |
| NOV37j | ------------------------------------------------------------ |
| NOV37k | CAHGCLNTQGSFKCVCHAGYELGADGRQCYRIEMEIVNSCEANNGGCSHGCSHTSAGPLC |
| NOV37l | CAHGCLNTQGSFKCVCHAGYELGADGRQCYRIEMEIVNSCEANNGGCSHGCSHTSAGPLC |
| NOV37m | CAHGCLNTQGSFKCVCHAGYELGADGRQCYRIEMEIVNSCEANNGGCSHGCSHTSAGPLC |
| NOV37n | CAHGCLNTQGSFKCVCHAGYELGADGRQCYRIEMEIVNSCEANNGGCSHGCSHTSAGPLC |
| NOV37o | CAHGCLNTQGSFKCVCHAGYELGADGRQCYRIEMEIVNSCEANNGGCSHGCSHTSAGPLC |
| NOV37a | ------------------------------------------------------------ |
| NOV37b | TCPRGYELDTDQRTCIRCPRLCRQPVLQQVCTNNPGGYECGCYAGYRLSADGCGCEDVDE |
| NOV37c | ------------------------------------------------------------ |
| NOV37d | TCPRGYELDTDQRTCIDVGDCADSPCCQQVCTNNPGGYECGCYAGYRLSADGCGCEDVDE |
| NOV37e | TCPRGYELDTDQRTCIRCRRLCRQPVLQQVCTNNPGGYECGCYAGYRLSADGCGCEDVDE |
| NOV37f | TCPRGYELDTDQRTCIRCRRLCRQPVLQQVCTNNPGGYECGCYAGYRLSADGCGCEDVDE |
| NOV37g | TCPRGYELDTDQRTCIRCRRLCRQPVLQQVCTNNPGGYECGCYAGYRLSADGCGCEDVDE |
| NOV37h | TCPRGYELDTDQRTCIRCRRLCRQPVLQQVCTNNPGGYECGCYAGYRLSADGCGCEDVDE |
| NOV37i | TCPRGYELDTDQRTCTRCRRLCRQPVLQQVCTNNPGGYECGCYAGYRLSADGCGCEDVDE |
| NOV37j | ------------------------------------------------------------ |
| NOV37k | TCPRGYELDTDQRTCIRCRRLCRQPVLQQVCTNNPGGYECGCYAGYRLSADGCGCEDVDE |
| NOV371 | TCPRGYELDTDQRTCIRCRRLCRQPVLQQVCTNNPGGYECGCYAGYRLSADGCGCEDVDE |
| NOV37m | TCPRGYELDTDQRTCIRCRRLCRQPVLQQVCTNNPGGYECGCYAGYRLSADGCGCEDVDE |
| NOV37n | TCPRGYELDTDQRTCIRCRRLCRQPVLQQVCTNNPGGYECGCYAGYRLSADGCGCEDVDE |
| NOV37o | TCPRGYELDTDQRTCIRCRRLCRQPVLQQVCTNNPGGYECGCYAGYRLSADGCGCEDVDE |
| NOV37a | ------------------------------------------------------------ |
| NOV37b | CASSRGGCEHHCTNLAGSFQCSCEAGYRLHEDRRGCSALEEPMVDLDGELPFVRPLPHIA |
| NOV37c | ------------------------------------------------------------ |
| NOV37d | CASSRGGCEHHCTNLAGSFQCSCEAGYRLHEDRRGCSPLEEPMVDLDGELPFVRPLPHIA |
| NOV37e | CASSRGGCEHHCTNLAGSFQCSCEAGYRLHEDRRGCSALEEPMVDLDGELPFVRPLPHIA |
| NOV37f | CASSRGGCEHHCTNLAGSFQCSCEAGYRLHEDRRGCSALEEPMVDLDGELPFVRPLPHIA |
| NOV37g | CASSRGGCEHHCTNLAGSFQCSCEAGYRLHEDRRGCSALEEPMVDLDGELPFVRPLPHIA |
| NOV37h | CASSRGGCEHHCTNLAGSFQCSCEAGYRLHEDRRGCSALEEPMVDLDGELPFVRPLPHIA |
| NOV37i | CASSRGGCEHHCTNLAGSFQCSCEAGYRLHEDRRGCSALEEPMVDLDGELPFVRPLPHIA |
| NOV37j | ------------------------------------------------------------ |
| NOV37k | CASSRGGCEHHCTNLAGSFQCSCEAGYRLHEDRRGCSALEEPMVDLDGELPFVRPLPHIA |
| NOV37l | CASSRGGCEHHCTNLAGSFQCSCEAGYRLHEDRRGCSALEEPMVDLDGELPFVRPLPHIA |
| NOV37m | CASSRGGCEHHCTNLAGSFQCSCEAGYRIHEDRRGCSALEEPMVDLDGELPFVRPLPHIA |
| NOV37n | CASSRGGCEHHCTNLAGSFQCSCEAGYRLHEDRRGCSALEEPMVDLDGELPFVRPLPHIA |
| NOV37o | CASSRGGCEHHCTNLAGSFQCSCEAGYRIHEDRRGCSALEEPMVDLDGELPFVRPLPHIA |
| NOV37a | ------------------------------------------------------------ |
| NOV37b | VLQDELPQLFQDDDVGADEEEAELRGEHTLTEKFVCLDDSFGHDCSLTCDDCRNGGTCLL |
| NOV37c | ------------------------------------------------------------ |
| NOV37d | VLQDELPQLFQDDDvGADEEEAELRGEHTLTEKFVCLDDSFGHDCSLTCDDCRNGGTCLL |
| NOV37e | VLQDELPQLFQDDDVGADEEEAELRGEHTLTEKFVCLDDSFGHDCSLTCDDCRNGGTCLL |
| NOV37f | VLQDELPQLFQDDDVGADEEEAELRGEHTLTEKFVCLDDSFGHDCSLTCDDCRNGGTCLL |
| NOV37g | VLQDELPQLFQDDDVGADEEEAELRGEHTLTEKFVCLDDSFGHDCSLTCDDCRNGGTCLL |
| NOV37h | VLQDELPQLFQDDDVGADEEEAELRGEHTLTEKFVCLDDSFGHDCSLTCDDCRNGGTCLL |
| NOV37i | VLQDELPQLFQDDDVGADEEEAELRGEHTLTEKFVCLDDSFGHDCSLTCDDCRNGGTCLL |
| NOV37j | ------------------------------------------------------------ |
| NOV37k | VLQDELPQLFQDDDVGADEEEAELRGEHTLTEKFVCLDDSFGHDCSLTCDDCRNGGTCLL |
| NOV37l | VLQDELPQLFQDDDVGADEEEAELRGEHTLTEKFVCLDDSFGHDCSLTCDDCRNGGTCLL |
| NOV37m | VLQDELPQLFQDDDVGADEEEAELRGEHTLTEKFVCLDDSFGHDCSLTCDDCRNGGTCLL |
| NOV37n | VLQDELPQLFQDDDVGADEEEAELRGEHTLTEKFVCLDDSFGHDCSLTCDDCRNGGTCLL |
| NOV37o | VLQDELPQLFQDDDVGADEEEAELRGEHTLTEKFVCLDDSFGHDCSLTCDDCRNGGTCLL |
| NOV37a | ------------------------------------------------------------ |
| NOV37b | GLDGCDCPEGWTGLICNESCPPDTFGKNCSFSCSCQNGGTCDSVTGACRCPPGVSGTNCE |
| NOV37c | ------------------------------------------------------------ |
| NOV37d | GLDGCDCPEGWTGLICNETCPPDTFGKNCSFSCSCQNGGTCDSVTGACRCPPGVSGTNCE |
| NOV37e | GLDGCDCPEGWTGLICNESCPPDTFGKNCSFSCSCQNGGTCDSVTGACRCPPGVSGTNCE |
| NOV37f | GLDGCDCPEGWTGLICNESCPPDTFGKNCSFSCSCQNGGTCDSVTGACRCPPGVSGTNCE |
| NOV37g | GLDGCDCPEGWTGLICNESCPPDTFGKNCSFSCSCQNGGTCDSVTGACRCPPGVSGTNCE |
| NOV37h | GLDGCDCPEGWTGLICNESCPPDTFGKNCSFSCSCQNGGTCDSVTGACRCPPGVSGTNCE |
| NOV37i | GLDGCDCPEGWTGLICNESCPPDTFGKNCSFSCSCQNGGTCDSVTGACRCPPGVSGTNCE |
| NOV37j | ------------------------------------------------------------ |
| NOV37k | GLDGCDCPEGWTGLICNESCPPDTFGKNCSFSCSCQNGGTCDSVTGACRCPPGVSGTNCE |
| NOV371 | GLDGCDCPEGWTGLICNESCPPDTFGKNCSFSCSCQNGGTCDSVTGACRCPPGVSGTNCE |
| NOV37m | GLDGCDCPEGWTGLICNESCPPDTFGKNCSFSCSCQNGGTCDSVTGACRCPPGVSGTNCE |
| NOV37n | GLDGCDCPEGWTGLICNESCPPDTFGKNCSFSCSCQNGGTCDSVTGACRCPPGVSGTNCE |
| NOV37o | GLDGCDCPEGWTGLICNESCPPDTFGKNCSFSCSCQNGGTCDSVTGACRCPPGVSGTNCE |
| NOV37a | ------------------------------------------------------------ |
| NOV37b | DGCPKGYYGKHCRKKCNCANRGRCHRLYGACLCDPGLYGRFCHLACPPWAFGPGCSEECQ |
| NOV37c | ------------------------------------------------------------ |
| NOV37d | DGCPKGYYGKHCRKKCNCANRGRCHRLYGACLCDPGLYGRFCHLTCPPWAFGPGCSEECQ |
| NOV37e | DGCPKGYYGKHCRKKCNCANRGRCHRLYGACLCDPGLYGRFCHLACPPWAFGPGCSEECQ |
| NOV37f | DGCPKGYYGKHCRKKCNCANRGRCHRLYGACLCDPGLYGRFCHLACPPWAFGPGCSEECQ |
| NOV37g | DGCPKGYYGKHCRKKCNCANRGRCHRLYGACLCDPGLYGRFCHLACPPWAFGPGCSEECQ |
| NOV37h | DGCPKGYYGKHCRKKCNCANRGRCHRLYGACLCDPGLYGRFCHLACPPWAFGPGCSEECQ |
| NOV37i | DGCPKGYYGKHCRKKCNCANRGRCHRLYGACLCDPGLYGRFCHLACPPWAFGPGCSEECQ |
| NOV37j | ------------------------------------------------------------ |
| NOV37k | DGCPKGYYGKHCRKKCNCANRGRCHRLYGACLCDPGLYGRFCHLACPPWAFGPGCSEECQ |
| NOV37l | DGCPKGYYGKHCRKKCNCANRGRCHRLYGACLCDPGLYGRFCHLACPPWAFGPGCSEECQ |
| NOV37m | DGCPKGYYGKHCRKKCNCANRGRCHRLYGACLCDPGLYGRFCHLACPPWAFGPGCSEECQ |
| NOV37n | DGCPKGYYGKHCRKKCNCANRGRCHRLYGACLCDPGLYGRFCHLACPPWAFGPGCSEECQ |
| NOV37o | DGCPKGYYGKHCRKKCNCANRGRCHRLYGACLCDPGLYGRFCHLACPPWAFGPGCSEECQ |
| NOV37a | ------------------------------------------------------------ |
| NOV37b | CVQPHTQSCDKRDGSCSCKAGFRGERCQAECEPGYFGPGCWQACTCPVGVACDSVSGECG |
| NOV37c | ------------------------------------------------------------ |
| NOV37d | CVQPHTQSCDKRDGSCSCKAGFRGERCQAECEPGYFGPGCWQACTCPVGVACDSVSGECG |
| NOV37e | CVQPHTQSCDKRDGSCSCKAGFRGERCQAECEPGYFGPGCWQACTCPVGVACDSVSGECG |
| NOV37f | CVQPHTQSCDKRDGSCSCKAGFRGERCQAECEPGYFGPGCWQACTCPVGVACDSVSGECG |
| NOV37g | CVQPHTQSCDKRDGSCSCKAGFRGERCQAECEPGYFGPGCWQACTCPVGVACDSVSGECG |
| NOV37h | CVQPHTQSCDKRDGSCSCKAGFRGERCQAECEPGYFGPGCWQACTCPVGVACDSVSGECG |
| NOV37i | CVQPHTQSCDKRDGSCSCKAGFRGERCQAECELGYFGPGCWQACTCPVGVACDSVSGECG |
| NOV371 | ------------------------------------------------------------ |
| NOV37k | CVQPHTQSCDKRDGSCSCKAGFRGERCQAECEPGYFGPGCWQACTCPVGVACDSVSGECG |
| NOV37l | CVQPHTQSCDKRDGSCSCKAGFRGERCQAECELGYFGPGCWQACTCPVGVACDSVSGECG |
| NOV37m | CVQPHTQSCDKRDGSCSCKAGFRGERCQAECEPGYFGPGCWQACTCPVGVACDSVSGECG |
| NOV37n | CVQPHTQSCDKRDGSCSCKAGFRGERCQAECEPGYFGPGCWQACTCPVGVACDSVSGECG |
| NOV37o | CVQPHTQSCDKRDGSCSCKAGFRGERCQAECEPGYFGPGCWQACTCPVGVACDSVSGECG |
| NOV37a | ------------------------------------------------------------ |
| NOV37b | KRCPAGFQGEDCGQECPVGTFGVNCSSSCSCGGAPCHGVTGQCRCPPGRTGEDCEAGECE |
| NOV37c | ------------------------------------------------------------ |
| NOV37d | KRCPAGFQGEDCGQECPVGTFGVNCSSSCSCGGAPCHGVTGQCRCPPGRTGEDCEADCPE |
| NOV37e | KRCPAGFQGEDCGQECPVGTFGVNCSSSCSCGGAPCHGVTGQCRCPPGRTGEDCEAGECE |
| NOV37f | KRCPAGFQGEDCGQECPVGTFGVNCSSSCSCGGAPCHGVTGQCRCPPGRTGEDCEAGECE |
| NOV37g | KRCPAGFQGEDCGQECPVGTFGVNCSSSCSCGGAPCHGVTGQCRCPPGRTGEDCEAGECE |
| NOV37h | KRCPAGFQGEDCGQECPVGTFGVNCSSSCSCGGAPCHGVTGQCRCPPGRTGEDCEAGECE |
| NOV37i | KRCPAGFQGEDCGQECPVGTFGVNCSSSCSCGGAPCHGVTGQCRCPPGRTGEDCEADCPE |
| NOV37j | ------------------------------------------------------------ |
| NOV37k | KRCPAGFQGEDCGQECPVGTFGVNCSSSCSCGGAPCHGVTGQCRCPPGRTGEDCEAGECE |
| NOV37l | KRCPAGFQGEDCGQECPVGTFGVNCSSSCSCGGAPCHGVTGQCRCPPGRTGEDCEADCPE |
| NOV37m | KRCPAGFQGEDCGQECPVGTFGVNCSSSCSCGGAPCHGVTGQCRCPPGRTGEDCEAGECE |
| NOV37n | KRCPAGFQGEDCGQECPVGTFGVNCSSSCSCGGAPCHGVTGQCRCPPGRTGEDCEAGECE |
| NOV37o | KRCPAGFQGEDCGQECPVGTFGVNCSSSCSCGGAPCHGVTGQCRCPPGRTGEDCEAGECE |
| NOV37a | ------------------------------------------------------------ |
| NOV37b | GLWGLGCQEICPACHNAARCDPETGACLCLPGFVGSRCQD-CEAGWYGPSCQTMCSCAND |
| NOV37c | ------------------------------------------------------------ |
| NOV37d | GRWGLGCQEICPACQHAARCDPETGACLCLPDFVGSRCQDVCPAGWYGPSCQTRCSCAND |
| NOV37e | GLWGLGCQEICPACHNAARCDPETGACLCLPGFVGSRCQD-CEAGWYGPSCQTMCSCAND |
| NOV37f | GLWGLGCQEICPACHNAARCDPETGACLCLPGFVGSRCQD-CEAGWYGPSCQTMCSCAND |
| NOV37g | GLWGLGCQEICPACHNAARCDPETGACLCLPGFVGSRCQD-CEAGWYGPSCQTMCSCAND |
| NOV37h | GLWGLGCQEICPACHNAARCDPETGACLCLPGFVGSRCQD-CEAGWYGPSCQTMCSCAND |
| NOV37i | GRWGLGCQEICPACQHAARCDPETGACLCLPGFVGSRCQDVCPAGWYGPSCQTRCSCAND |
| NOV37j | ------------------------------------------------------------ |
| NOV37k | GLWGLGCQEICPACHNAARCDPETGACLCLPGFVGSRCQD-CEAGWYGPSCQTMCSCAND |
| NOV37l | GRWGLGCQEICPACQHAARCDPETGACLCLPGFVGSRCQDVCPAGWYGPSCQTRCSCAND |
| NOV37m | GLWGLGCQEICPACHNAARCDPETGACLCLPGFVGSRCQD-CEAGWYGPSCQTMCSCAND |
| NOV37n | GLWGLGCQEICPACHNAARCDPETGACLCLPGFVGSRCQD-CEAGWYGPSCQTMCSCAND |
| NOV37o | GLWGLGCQEICPACHNAARCDPETGACLCLPGFVGSRCQD-CEAGWYGPSCQTMCSCAND |
| NOV37a | -------------------MAPASAPLAAGAPAVPRPALPACTATTVGIPASARTEGPVT |
| NOV37b | GHCHQDTGHCSCAPGWTGFSCQRACDTGHWGPDCSHPCNCSAGHGSCDAISGLCLCEAGY |
| NOV37c | ----------DPCLAGPPLMAPASAPLAAGAPAVPRPALPACTATTVGIPASARTEGPVT |
| NOV37d | GHCHPATGHCSCAPGWTGFSCQRACDTGHWGPDCSHPCNCSAGHGSCDAISGLCLCEAGY |
| NOV37e | GHCHQDTGHCSCAPGWTGFSCQRACDTGHWGPDCSHPCNCSAGHGSCDAISGLCLCEAGY |
| NOV37f | GHCHQDTGHCSCAPGWTGFSCQRACDTGHWGPDCSHPCNCSAGHGSCDAISGLCLCEAGY |
| NOV37g | GHCHQDTGHCSCAPGWTGFSCQRACDTGHWGPDCSHPCNCSAGHGSCDAISGLCLCEAGY |
| NOV37h | GHCHQDTGHCSCAPGWTGFSCQRACDTGHWGPDCSHPCNCSAGHGSCDAISGLCLCEAGY |
| NOV37i | GHCHPATGHCSCAPGWTGFSCQPACDTGHWGPDCSHPCNCSAGHGSCDAISGLCLCEAGY |
| NOV37j | -------------------MAPASAPLAAGAPAVPRPALPACTATTVGIPASARTEGPVT |
| NOV37k | GHCHQDTGHCSCAPGWTGFSCQEACDTGHWGPDCSHPCNCSAGHGSCDAISGLCLCEAGY |
| NOV37l | GHCHPATGHCSCAPGWTGFSCQEACDTGHWGPDCSHPCNCSAGHGSCDAISGLCLCEAGY |
| NOV37m | GHCHQDTGHCSCAPGWTGFSCQRACDTGHWGPDCSHPCNCSAGHGSCDAISGLCLCEAGY |
| NOV37n | GHCHQDTGHCSCAPGWTGFSCQRACDTGHWGPDCSHPCNCSAGHGSCDAISGLCLCEAGY |
| NOV37o | GHCHQDTGHCSCAPGWTGFSCQRACDTGHWGPDCSHPCNCSAGHGSCDAISGLCLCEAGY |
| NOV37a | LSQACEH-PCPPGFHGAGRQGLCWCQHGAPCDPISGRCLCPAGFHGHFCERDCRRGQFGP |
| NOV37b | VGPRCEQSECPQGHFGPGCEQRCQCQHGAACDHVSGACTCPAGWRGTFCEHACPAGFFGL |
| NOV37c | LSQACEH-PCPPGFHGAGCQGLCWCQHGAPCDPISGRCLCPAGFHGHFCERDCRRGQFGP |
| NOV37d | VGPRCEQ-QCPQGHFGPGCEQLCQCQHGAACDHVSGACTCPAGWRGTFCEHACPAGFFGL |
| NOV37e | VGPRCEQSECPQGHFGPGCEQRCQCQHGAACDHVSGACTCPAGWRGTFCEHACPAGFFGL |
| NOV37f | VGPRCEQSECPQGHFGPGCEQRCQCQHGAACDHVSGACTCPAGWRGTFCEHACPAGFFGL |
| NOV37g | VGPRCEQSECPQGHFGPGCEQRCQCQHGAACDHVSGACTCPAGWRGTFCEHACPAGFFGL |
| NOV37h | VGPRCEQSECPQGHFGPGCEQRCQCQHGAACDHVSGACTCPAGWRGTFCEHACPAGFFGL |
| NOV37i | VGPRCEQ-QCpQGHFGPGCEQLCQCQHGAACDHVSGACTCPAGWRGTFCEHACPAGFFGL |
| NOV37j | LSQACEH-PCPPGFHGAGCQGLCWCQHGAPCDPISGRCLCPAGFHGHFCERDCRRGQFGP |
| NOV37k | VGPRCEQSECPQGHFGPGCEQRCQCQHGAACDHVSGACTCPAGWRGTFCEHACPAGFFGL |
| NOV37l | VGPRCEQ-QCPQGHFGPGCEQLCQCQHGAACDHVSGACTCPAGWRGTFCEHACPAGFFGL |
| NOV37m | VGPRCEQSECPQGHFGPGCEQRCQCQHGAACDHVSGACTCPAGWRGTFCEHACPAGFFGL |
| NOV37n | VGPRCEQSECPQGHFGPGCEQRCQCQHGAACDHVSGACTCPAGWRGTFCEHACPAGFFGL |
| NOV37o | VGPRCEQSECPQGHFGPGCEQRCQCQHGAACDHVSGACTCPAGWRGTFCEHACPAGFFGL |
| NOV37a | SCTLHCDCGGGADCDPVSGQCHCVDGYMGPTCREG------------------------- |
| NOV37b | DCRSACNCTAGAACDAVNGSCLCPAGRRGPRCAESACPAHTYGHNCSQACACFNGASCDP |
| NOV37c | SCTLHCDCGGGADCDPVSGQCHCVDGYMGPTCREG------------------------- |
| NOV37d | DCRSACNCTAGAACDAVNGSCLCPAGRRGPRCAEK------------------------- |
| NOV37e | DCRSACNCTAGAACDAVNGSCLCPAGRRGPRCAESACPAHTYGHNCSQACACFNGASCDP |
| NOV37f | DCRSACNCTAGAACDAVNGSCLCPAGRRGPRCAESACPAHTYGHNCSQACACFNGASCDP |
| NOV37g | DCRSACNCTAGAACDAVNGSCLCPAGRRGPRCAESACPAHTYGHNCSQACACFNGASCDP |
| NOV37h | DCRSACNCTAGAACDAVNGSCLCPAGRRGPRCAESACPAHTYGHNCSQACACFNGASCDP |
| NOV37i | DCRSACNCTAGAACDAVNGSCLCPAGRRGPRCAET------------------------- |
| NOV37j | SCTLHCDCGGGADCDPVSGQCHCVDGYMGPTCREG------------------------- |
| NOV37k | DCRSACNCTAGAACDAVNGSCLCPAGRRGPRCAET-CPAHTYGHNCSQACACFNGASCDP |
| NOV37l | DCRSACNCTAGAACDAVNGSCLCPAGRRGPRCAE------T------------------- |
| NOV37m | DCRSACNCTAGAACDAVNGSCLCPAGRRGPRCAESACPAHTYGHNCSQACACFNGASCDP |
| NOV37n | DCRSACNCTAGAACDAVNGSCLCPAGRRGPRCAESACPAHTYGHNCSQACACFNGASCDP |
| NOV37o | DCRSACNCTAGAACDAVNGSCLCPAGRRGPRCAESACPAHTYGHNCSQACACFNGASCDP |
| NOV37a | ---------------------------------------------GPLRIPENPSLAQGS |
| NOV37b | VHGQCHCAPGWMGPSCLQACPAGLYGDNCRHSCLCQNGGTCDPVSGHCACPEGWAGLACE |
| NOV37c | ---------------------------------------------GPLRLPENPSLAQGS |
| NOV37d | ------------------------------------------------------------ |
| NOV37e | VHGQCHCAPGWMGPSCLQACPAGLYGDNCRHSCLCQNGGTCDPVSGHCACPEGWAGLACE |
| NOV37f | VHGQCHCAPGWMGPSCLQACPAGLYGDNCRHSCLCQNGGTCDPVSGHCACPEGWAGLACE |
| NOV37g | VHGQCHCAPGWMGPSCLQACPAGLYGDNCRHSCLCQNGGTCDPVSGHCACPEGWAGLACE |
| NOV37h | VHGQCHCAPGWMGPSCLQACPAGLYGDNCRHSCLCQNGGTCDPVSGHCACPEGWAGLACE |
| NOV37i | -------------------CPAGLYGDNCRHSCLCQNGGTCDPVSGHCACPEGWAGLACE |
| NOV37j | ---------------------------------------------GPLRLPENPSLAQGS |
| NOV37k | VHGQCHCAPGWMGPSCLQACPAGLYGDNCRHSCLCQNGGTCDPVSGHCACPEGWAGLACE |
| NOV37l | -------------------CPAGLYGDNCRHSCLCQNGGTCDPVSGHCACPEGWAGLACE |
| NOV37m | VHGQCHCAPGWMGPSCLQACPAGLYGDNCRHSCLCQNGGTCDPVSGHCACPEGWAGLACE |
| NOV37n | VHGQCHCAPGWMGPSCLQACPAGLYGDNCRHSCLCQNGGTCDPVSGHCACPEGWAGLACE |
| NOV37o | VHGQCHCAPGWMGPSCLQACPAGLYGDNCRHSCLCQNGGTCDPVSGHCACPEGWAGLACE |
| NOV37a | AGTLPASSRPTSRSGGPARH---------------------------------------- |
| NOV37b | VECLPRDVRAGCRHSGGCLNGGLCDPHTGRCLCPAGWTGDKCQSPAACAKGTFGPHCEGR |
| NOV37c | AGTLPASSRPTSRS---------------------------------------------- |
| NOV37d | --CLPRDVRAGCRHSGGCLNGGLCDPHTGRCLCPAGWTGDKCQSP--------------- |
| NOV37e | VECLPRDVRAGCRHSGGCLNGGLCDPHTGRCLCPAGWTGDKCQSPAACAKGTFGPHCEGR |
| NOV37f | VECLPRDVRAGCRHSGGCLNGGLCDPHTGRCLCPAGWTGDKCQSPAACAKGTFGPHCEGR |
| NOV37g | VECLPRDVRAGCRHSGGCLNGGLCDPHTGRCLCPAGWTGDKCQSPAACAKGTFGPHCEGR |
| NOV37h | VECLPRDVRAGCRHSGGCLNGGLCDPHTGRCLCPAGWTGDKCQSPAACAKGTFGPHCEGR |
| NOV37i | KECPPRDVRAGCRHSGGCLNGGLCDPHTGRCLCPAGWAGDKCQS---------------- |
| NOV37j | AGTLPASSRPTS------------------------------------------------ |
| NOV37k | VECLPRDVRAGCRHSGGCLNGGLCDPHTGRCLCPAGWTGDKCQSPAACAKGTFGPHCEGR |
| NOV37l | KECPPRDVRAGCRHSGGCLNGGLCDPHTGRCLCPAGWTGDKCQS---------------- |
| NOV37m | VECLPRDVRAGCRHSGGCLNGGLCDPHTGRCLCPAGWTGDKCQSPAACAKGTFGPHCEGR |
| NOV37n | VECLPRDVRAGCRHSGGCLNGGLCDPHTGRCLCPAGWTGDKCQSPAACAKGTFGPHCEGR |
| NOV37o | VECLPRDVRAGCRHSGGCLNGGLCDPHTGRCLCPAGWTGDKCQSPAACAKGTFGPHCEGR |
| NOV37a | ------------------------------------------------------------ |
| NOV37b | CACRWGGPCHLATGACLCPPGWRGPHLSAACLRGWFGEACAQRCSCPPGAACHHVTGACR |
| NOV37c | ------------------------------------------------------------ |
| NOV37d | ----------------------------CLRG--WFGEACAQRCSCPPGAACHHVTGACR |
| NOV37e | CACRWGGPCHLATGACLCPPGWRGPHLSAACLRGWFGEACAQRCSCPPGAACHHVTGACR |
| NOV37f | CACRWGGPCHLATGACLCPPGWRGPHLSAACLRGWFGEACAQRCSCPPGAACHHVTGACR |
| NOV37g | CACRWGGPCHLATGACLCPPGWRGPHLSAACLRGWFGEACAQRCSCPPGAACHHVTGACR |
| NOV37h | CACRWGGPCHLATGACLCPPGWRGPHLSAACLRGWFGEACAQRCSCPPGAACHHVTGACR |
| NOV37i | -----------P----------------CLRG--WFGEACAQHCSCPPGAACHHVTGACR |
| NOV37j | ------------------------------------------------------------ |
| NOV37k | CACRWGGPCHLATGACLCPPGWRGPHLSAACLRGWFGEACAQRCSCPPGAACHHVTGACR |
| NOV37l | --HP------------------HGPLLEASAALIFLQPACGAGLERPVPSAA-----AAR |
| NOV37m | CACRWGGPCHLATGACLCPPGWRGPHLSAACLRGWFGEACAQRCSCPPGAACHHVTGACR |
| NOV37n | CACRWGGPCHLATGACLCPPGWRGPHLSAACLRGWFGEACAQRCSCPPGAACHHVTGACR |
| NOV37o | CACRWGGPCHLATGACLCPPGWRGPHLSAACLRGWFGEACAQRCSCPPGAACHHVTGACR |
| NOV37a | ------------------------------------------------------------ |
| NOV37b | CPPGFTGSGCEQACPPGSFGEDCAQMCQCPGENPACHPATGTCSCAAGYHGPSCQQRCPP |
| NOV37c | ------------------------------------------------------------ |
| NOV37d | CPPGFTGSGCEQGCP--------------------------------------------P |
| NOV37e | CPPGFTGSGCEQACPPGSFGEDCAQMCQCPGENPACHPATGTCSCAAGYHGPSCQQRCPP |
| NOV37f | CPPGFTGSGCEQACPPGSFGEDCAQMCQCPGENPACHPATGTCSCAAGYHGPSCQQRCPP |
| NOV37g | CPPGFTGSGCEQACPPGSFGEDCAQMCQCPGENPACHPATGTCSCAAGYHGPSCQQRCPP |
| NOV37h | CPPGFTGSGCEQACPPGSFGEDCAQMCQCPGENPACHPATGTCSCAAGYHGPSCQQRCPP |
| NOV37i | CPPGFTGSGCEQGCP--------------------------------------------P |
| NOV37j | ------------------------------------------------------------ |
| NOV37k | CPPGFTGSGCEQACPPGSFGEDCAQMCQCPGENPACHPATGTCSCAAGYHGPSCQQRCPP |
| NOV37l | LPLPATTSLGPAAVPLASL---------------A----------------PAASRDVRP |
| NOV37m | CPPGFTGSGCEQACPPGSFGEDCAQMCQCPGENPACHPATGTCSCAAGYHGPSCQQRCPP |
| NOV37n | CPPGFTGSGCEQACPPGSFGEDCAQMCQCPGENPACHPATGTCSCAAGYHGPSCQQRCPP |
| NOV37o | CPPGFTGSGCEQACPPGSFGEDCAQMCQCPGENPACHPATGTCSCAAGYHGPSCQQRCPP |
| NOV37a | ------------------------------------------------------------ |
| NOV37b | GRYGPGCEQLCGCLNGGSCDAATGACRCPTGFLGTDCNLTCPQGRFGPNCTHVCGCGQGA |
| NOV37c | ------------------------------------------------------------ |
| NOV37d | GRYGPGCEQLCGCLNGGSCDAATGACRCPTGFLGTDCNLTCPQGRFGPNCTHVCGCGQGA |
| NOV37e | GRYGPGCEQLCGCLNGGSCDAATGACRCPTGFLGTDCNLTCPQGRFGPNCTHVCGCGQGA |
| NOV37f | GRYGPGCEQLCGCLNGGSCDAATGACRCPTGFLGTDCNLTCPQGRFGPNCTHVCGCGQGA |
| NOV37g | GRYGPGCEQLCGCLNGGSCDAATGACRCPTGFLGTDCNLTCPQGRFGPNCTHVCGCGQGA |
| NOV37h | GRYGPGCEQLCGCLNGGSCDAATGACRCPTGFLGTDCNLTCPQGRFGPNCTHVCGCGQGA |
| NOV37i | GRYGPGCEQLCGCLNGGSCDAATGACRCPTGFLGTDCNLTCPQGRFGPNCTHVCGCGQGA |
| NOV37j | ------------------------------------------------------------ |
| NOV37k | GRYGPGCEQLCGCLNGGSCDAATGACRCPTGFLGTDCNLTCPQGRFGPNCTHVCGCGQGA |
| NOV37l | GGMGQAVNSCVGVSTGAPVMRPRGPAAAPLGSSGR--TATSP--------------VRPA |
| NOV37m | GRYGPGCEQLCGCLNGGSCDAATGACRCPTGFLGTDCNLTCPQGRFGPNCTHVCGCGQGA |
| NOV37n | GRYGPGCEQLCGCLNGGSCDAATGACRCPTGFLGTDCNLTCPQGRFGPNCTHVCGCGQGA |
| NOV37o | GRYGPGCEQLCGCLNGGSCDAATGACRCPTGFLGTDCNLTCPQGRFGPNCTHVCGCGQGA |
| NOV37a | ------------------------------------------------------------ |
| NOV37b | ACDPVTGTCLCPPGRAGVRCERGCPQNRFGVGCEHTCSCRNGGLCHASGACATPASGSCS |
| NOV37c | ------------------------------------------------------------ |
| NOV37d | ACDPVTGTCLCPPGRAGVRCERGCPQNRFGVGCEHTCSCRNGGLCHAS-------NGSCS |
| NOV37e | ACDPVTGTCLCPPGRAGVRCERGCPQNRFGVGCEHTCSCRNGGLCHAS-------NGSCS |
| NOV37f | ACDPVTGTCLCPPGRAGVRCERGCPQNRFGVGCEHTCSCRNGGLCHAS-------NGSCS |
| NOV37g | ACDPVTGTCLCPPGRAGVRCERGCPQNRFGVGCEHTCSCRNGGLCHAS-------KRQLL |
| NOV37h | ACDPVTGTCLCPPGRAGVRCERGCPQNRFGVGCEHTCSCRNGGLCHAS-------KRQLL |
| NOV37i | ACDPVTGTCLCPPGRAGVRCERGCPQNRFGVGCEHTCSCRNGGLCHAS-------NGSCS |
| NOV37j | ------------------------------------------------------------ |
| NOV37k | ACDPVTGTCLCPPGRAGVRCERGCPQNRPGVGCEHTCSCRNGGLCHAS-------KRQLL |
| NOV37l | ASAPTAPTCVVVG--------RGRPATL-------------------------------- |
| NOV37m | ACDPVTGTCLCPPGRAGVRCERGCPQNRFGVGCEHTCSCRNGGLCHAS-------NGSCS |
| NOV37n | ACDPVTGTCLCPPGPAGVRCERGCPQNRFGVGCEHTCSCPNGGLCHAS-------NGSCS |
| NOV37o | ACDPVTGTCLCPPGRAGVRCERGCPQNRFGVGCEHTCSCPNGGLCHAS-------NGSCS |
| NOV37a | ------------------------------------------------------------ |
| NOV37b | CGLGWTGRHCELACPPGRYGAACHLECSCHNNSTCEPATGTCRCGPGFYGQACEHPCPPG |
| NOV37c | ------------------------------------------------------------ |
| NOV37d | CGLGWTGRHCELACPPGRYGAACHLECSCHNNSTCEPATGTCRCGPGFYGQACEHPCPPG |
| NOV37e | CGLGWTGRHCELACPPGRYGAACHLECSCHNNSTCEPATGTCRCGPGFYGQACEHPCPPG |
| NOV37f | CGLGWTGRHCELACPPGRYGAACHLECSCHNNSTCEPATGTCRCGPGFYGQACEHPCPPG |
| NOV37g | LWPGLDGAALRAGLSPWALRSRLPSGVLLPQQQHV------------------------- |
| NOV37h | LWPGLDGAALRAGLSPWALRSRLPSGVLLPQQQHV------------------------- |
| NOV37i | CGLGWTGRHCELACPPGRYGAACHLECSCHNNSTGEPATGTCRCGPGFYGQACEHPCPPG |
| NOV37j | ------------------------------------------------------------ |
| NOV37k | LWPGLDGAALRAGLSPWALRSRLPSGVLLPQQQHV------------------------- |
| NOV37l | ------------------------------------------------------------ |
| NOV37m | CGLGWTGRHCELACPPGRYGAACHLECSCHNNSTCEPATGTCRCGPGFYGQACEHPCPPG |
| NOV37n | CGLGWTGRHCELACPPGRYGAACHLECSCHNNSTCEPATGTCRCGPGFYGQACEHPCPPG |
| NOV37o | CGLGWTGRHCELACPPGRYGAACHLECSCHNNSTCEPATGTCRCGPGFYGQACEHPCPPG |
| NOV37a | ------------------------------------------------------------ |
| NOV37b | FHGAGCQGLCWCQHGAPCDPISGRCLCPAGFHGHFCERGCEPGSFGEGCHQRCDCDGGAP |
| NOV37c | ------------------------------------------------------------ |
| NOV37d | FHGAGCQGLCWCQHGAPCDPISGRCLCPAGFHGHFCERGCEPGSFGEGCHQRCDCDGGAP |
| NOV37e | FHGAGCQGLCWCQHGAPCDPISGRCLCPAGFHGHFCERGCEPGSFGEGCHQRCDCDGGAP |
| NOV37f | FHGAGCQGLCWCQHGAPCDPISGRCLCPAGFHGHFCERGCEPGSFGEGCHQRCDCDGGAP |
| NOV37g | ------------------------------------------------------------ |
| NOV37h | ------------------------------------------------------------ |
| NOV37i | FHGAGCQGLCWCQHGAPCDPISGRCLCPAGFHGHFCERGCEPGSFGEGCHQRCDCDGGAP |
| NOV37j | ------------------------------------------------------------ |
| NOV37k | ------------------------------------------------------------ |
| NOV37l | ------------------------------------------------------------ |
| NOV37m | FHGAGCQGLCWCQHGAPCDPISGRCLCPAGFHGHFCERGCEPGSFGEGCHQRCDCDGGAP |
| NOV37n | FHGAGCQGLCWCQHGAPCDPISGRCLCPAGFHGHFCERGCEPGSFGEGCHQRCDCDGGAP |
| NOV37o | FHGAGCQGLCWCQHGAPCDPISGRCLCPAGFHGHFCERGCEPGSFGEGCHQRCDCDGGAP |
| NOV37a | ------------------------------------------------------------ |
| NOV37b | CDPVTGLCLCPPGRSGATCNLDCRRGQFGPSCTLHCDCGGGADCDPVSGQCHCVDGYMGP |
| NOV37c | ------------------------------------------------------------ |
| NOV37d | CDPVTGLCLCPPGRSGATCNLDCRRGQFGPSCTLHCDCGGGADCDPVSGQCHCVDGYMGP |
| NOV37e | CDPVTGLCLCPPGRSGATCNLDCRRGQFGPSCTLHCDCGGGADCDPVSGQCHCVDGYMGP |
| NOV37f | CDPVTGLCLCPPGRSGATCNLDCRRGQFGPSCTLHCDCGGGADCDPVSGQCHCVDGYMGP |
| NOV37g | ------------------------------------------------------------ |
| NOV37h | ------------------------------------------------------------ |
| NOV37i | CDPVTGLCLCPPGRSGATCNLDCRRGQFGPSCTLHCDCGGGADCDPVSGQCHCVDGYMGP |
| NOV37j | ------------------------------------------------------------ |
| NOV37k | ------------------------------------------------------------ |
| NOV37l | ------------------------------------------------------------ |
| NOV37m | CDPVTGLCLCPPGRSGATCNLDCRRGQFGPSCTLHCDCGGGADCDPVSGQCHCVDGYMGP |
| NOV37n | CDPVTGLCLCPPGRSGATCNLDCRRGQFGPSCTLHCDCGGGADCDPVSGQCHCVDGYMGP |
| NOV37o | CDPVTGLCLCPPGRSGATCNLDCRRGQFGPSCTLHCDCGGGADCDPVSGQCHCVDGYMGP |
| NOV37a | ------------------------------------------------------------ |
| NOV37b | TCREGGPLRLPENPSLAQGSAGTLPASSRPTSRSGGPARH-------------------- |
| NOV37c | ------------------------------------------------------------ |
| NOV37d | TCREGGPLRLPENPSLAQGSAGTLPASSRPTSRSGGPARHGTG----------------- |
| NOV37e | TCREAG----------------TLPASSRPTSRSGGPARHVFPGLEGKPIPNPLLGLDST |
| NOV37f | TCREAG----------------TLPASSRPTSRSGGPARHEFPGLEGKPIPNPLLGLDST |
| NOV37g | ------------------------------------------------------------ |
| NOV37h | ------------------------------------------------------------ |
| NOV37i | TCREGGPLRLPENPSLLAQGSAGTLPASSRPTSRSGGPARH------------------- |
| NOV37j | ------------------------------------------------------------ |
| NOV37k | ------------------------------------------------------------ |
| NOV37l | ------------------------------------------------------------ |
| NOV37m | TCREAG----------TLPASSRPTSRSGGPARH-------------------------- |
| NOV37n | TCREAG----------TLPASSRPTSRSGGPARH-------------------------- |
| NOV37o | TCREAG----------TLPASSRPTSRSGGPARH-------------------------- |
| NOV37a | --------- |
| NOV37b | --------- |
| NOV37c | --------- |
| NOV37e | RTGHHHHHH |
| NOV37f | RTGHHHHHH |
| NOV37g | --------- |
| NOV37h | --------- |
| NOV37i | --------- |
| NOV37j | --------- |
| NOV37k | --------- |
| NOV37l | --------- |
| NOV37m | --------- |
| NOV37n | --------- |
| NOV37o | --------- |
| NOV37a | (SEQ ID NO: 858) |
| NOV37b | (SEQ ID NO: 860) |
| NOV37c | (SEQ ID NO: 862) |
| NOV37d | (SEQ ID NO: 864) |
| NOV37e | (SEQ ID NO: 866) |
| NOV37f | (SEQ ID NO: 868) |
| NOV37g | (SEQ ID NO: 870) |
| NOV37h | (SEQ ID NO: 872) |
| NOV37i | (SEQ ID NO: 874) |
| NOV37j | (SEQ ID NO: 876) |
| NOV37k | (SEQ ID NO: 878) |
| NOV37l | (SEQ ID NO: 880) |
| NOV37m | (SEQ ID NO: 882) |
| NOV37n | (SEQ ID NO: 884) |
| NOV37o | (SEQ ID NO: 886) |

Further analysis of the NOV37a protein yielded the following properties shown in Table 37C.

TABLE 37C
Protein Sequence Properties NOV37a

| | |
|---|---|
| SignalP analysis: | No Known Signal Sequence Predicted |
| PSORT II analysis: | |

| PSG: | a new signal peptide prediction method | |
|---|---|---|
| | N-region: | length 0; pos. chg 0; neg. chg 0 |
| | H-region: | length 16; peak value 6.60 |
| | PSG score: | 2.20 |

| | |
|---|---|
| GvH: | von Heijne's method for signal seq. recognition |
| | GvH score (threshold: −2.1): −5.33 |
| | possible cleavage site: between 14 and 15 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 0 | |
| | number of TMS(s) . . . fixed | |
| | PERIPHERAL | Likelihood = 5.83 (at 18) |

| | ALOM score: 5.83 (number of TMSs: 0) | | | |
|---|---|---|---|---|
| MTOP: | Prediction of membrane topology (Hartmann et al.) | | | |
| | Center position for calculation: 6 | | | |
| | Charge difference: 0.0 C(1.0)-N(1.0) | | | |
| | N >= C: N-terminal side will be inside | | | |
| MITDISC: | discrimination of mitochondrial targeting seq | | | |
| | R content: | 2 | Hyd Moment(75): | 1.11 |
| | Hyd Moment(95): | 2.89 | G content: | 2 |
| | D/E content: | 1 | S/T content: | 6 |

| | |
|---|---|
| | Score: −3.38 |
| Gavel: | prediction of cleavage sites for mitochondrial preseq |
| | R-2 motif at 45 ART\|EG |

NUCDISC: discrimination of nuclear localization signals

| | |
|---|---|
| | pat4: none |
| | pat7: none |
| | bipartite: none |
| | content of basic residues: 7.1% |
| | NLS Score: −0.47 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 94.1 |
| COIL: | Lupas's algorithm to detect coiled-coil regions |
| | total: 0 residues |

Final Results (k = 9/23):

```
65.2%: nuclear
21.7%: mitochondrial
  8.7%: cytoplasmic
  4.3%: extracellular, including cell wall
```

>> prediction for CG56449-04 is nuc (k = 23)

A search of the NOV37a protein against the Geneseq database, a proprietary database that contains sequences published in patents and patent publication, yielded several homologous proteins shown in Table 37D.

TABLE 37D
Geneseq Results for NOV37a

| | | NOV37a | Identities/ | | |
|---|---|---|---|---|---|
| | | Residues/ | Similarities for | | |
| Geneseq | Protein/Organism/Length | Match | the Matched | | Expect |
| Identifier | [Patent #, Date] | Residues | Region | | Value |
| AAY72716 | HMEIR04 clone human attractin-like | 38 . . . 170 | 126/176 | (71%) | 6e−73 |
| | protein - Homo sapiens, 310 aa. | 135 . . . 310 | 126/176 | (71%) | |
| | [WO200116156-A1, 08-MAR-2001] | | | | |
| AAY72715 | HFICU08 clone human attractin-like | 38 . . . 170 | 107/133 | (80%) | 4e−65 |
| | protein - Homo sapiens, 474 aa. | 342 . . . 474 | 108/133 | (80%) | |
| | [WO200116156-A1, 08-MAR-2001] | | | | |
| AAY72740 | HMEIR04 clone human attractin-like | 55 . . . 170 | 113/159 | (71%) | 1e−63 |
| | partial protein - Homo sapiens, 159 aa. | 1 . . . 159 | 113/159 | (71%) | |
| | [WO200116156-A1, 08-MAR-2001 ] | | | | |
| ABB72269 | Murine protein isolated from skin cells | 38 . . . 134 | 74/140 | (52%) | 4e−40 |
| | SEQ ID NO: 458 - Mus sp, 296 aa. | 135 . . . 274 | 79/140 | (55%) | |
| | [WO200190357-A1, 29-NOV-2001] | | | | |
| ABB72216 | Murine protein isolated from skin cells | 38 . . . 134 | 74/140 | (52%) | 4e−40 |
| | SEQ ID NO: 332 - Mus sp, 299 aa. | 138 . . . 277 | 79/140 | (55%) | |
| | [WO200190357-A1, 29-NOV-2001] | | | | |

In a BLAST search of public sequence databases, the NOV37a protein was found to have homology to the proteins shown in the BLASTP data in Table 37E.

TABLE 37E
Public BLASTP Results for NOV37a

| | | NOV37a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| O75095 | MEGF6 - Homo sapiens (Human), | 46 . . . 170 | 99/134 | (73%) | 5e−57 |
| | 1246 aa (fragment). | 1113 . . . 1246 | 105/134 | (77%) | |
| O88281 | MEGF6 - Rattus norvegicus (Rat), | 38 . . . 164 | 82/170 | (48%) | 1e−41 |
| | 1574 aa. | 1413 . . . 1571 | 93/170 | (54%) | |
| AAH39980 | Hypothetical protein - Mus musculus | 38 . . . 134 | 74/140 | (52%) | 1e−39 |
| | (Mouse), 546 aa. | 385 . . . 524 | 79/140 | (55%) | |
| Q8BKK7 | MEGF11 protein - Mus musculus | 46 . . . 137 | 45/92 | (48%) | 7e−23 |
| | (Mouse), 947 aa. | 183 . . . 274 | 54/92 | (57%) | |
| Q96KG6 | MEGF11 protein (Hypothetical | 46 . . . 137 | 45/92 | (48%) | 7e−23 |
| | protein KIAA1781) - Homo sapiens | 140 . . . 231 | 54/92 | (57%) | |
| | (Human), 969 aa. | | | | |

PFam analysis predicts that the NOV37a protein contains the domains shown in the Table 37F.

TABLE 37F
Domain Analysis of NOV37a

| | | Identities/ | |
|---|---|---|---|
| | | Similarities | |
| | NOV37a Match | for the | Expect |
| Pfam Domain | Region | Matched Region | Value |
| laminin_EGF | 63 . . . 102 | 17/60 (28%) | 0.37 |
| | | 30/60 (50%) | |
| EGF | 102 . . . 132 | 11/47 (23%) | 0.16 |
| | | 23/47 (49%) | |

### Example 38

The NOV38 clone was analyzed, and the nucleotide and encoded polypeptide sequences are shown in Table 38A.

TABLE 38A
NOV38 Sequence Analysis

| | | |
|---|---|---|
| NOV38a, CG56594-01 | SEQ ID NO: 889 | 695 bp |

| | | |
|---|---|---|
| DNA Sequence | ORF Start: ATG at 53 | ORF Stop: TGA at 662 |

GCACCCTGGCCCAGCTCTGAGTCCTGGGACCCTCGGTCCTCTCTCCTGGGCCATGGCCAACTCAGGCC
TCCAGCTCCTGGGCTACTTCTTGGCCCTGGGTGGCTGGGTGGGCATCATTGCTAGCACAGCCCTGCCA
CAGTGGAAGCAGTCTTCCTACGCAGGCGACGCCATCATCACTGCCGTGGGCCTCTATGAAGGGCTCTG
GATGTCCTGCGCCTCCCAGAGCACTGGGCAAGTGCAGTGCAAGCTCTACGACTCGCTGCTCGCCCTGG
ACGGTAGGCCCCAGGCCGCGCGGGCCCTGATGGTGGTGGCCGTGCTCCTGGGCTTCGTGGCCATGGTC
CTCAGCGTAGTTGGCATGAAGTGTACGCGGGTGGGAGACAGCAACCCCATTGCCAAGGGCCGTGTTGC
CCCTGGTGACCCAGGAGTTCTTCAACCCAGAATTTGGCCCAGCCCTGTTCGTGGGCTGGGCCTCAGCT
GGCCTGGCCGTGCTGGGCGGCTCCTTCCTCTGCTGCACATGCCCGGAGCCAGAGAGACCCAACAGCAG
CCCACAGCCCTATCGGCCTGGACCCTCTGCTGCTGCCCGAGAGTACGTCTGAGCTCCGCCTGCCCTGG
CCAGCCCCCCACCCA

NOV38a, CG56594-01

| | | | |
|---|---|---|---|
| Protein Sequence | SEQ ID NO: 890 | 203 aa | MW at 21174.4kD |

MANSGLQLLGYFLALGGWVGIIASTALPQWKQSSYAGDAIITAVGLYEGLWMSCASQSTGQVQCKLYD
SLLALDGRPQAARALMVVAVLLGFVAMVLSVVGMKCTRVGDSNPIAKGRVAIAGGALFILAGLCTLTA
VSWYATLVTQEFFNPEFGPALFVGWASAGLAVLGGSFLCCTCPEPERPNSSPQPYRPGPSAAAREYV

| | | |
|---|---|---|
| NOV38b, 277685552 | SEQ ID NO: 891 | 655 bp |

| | | |
|---|---|---|
| DNA Sequence | ORF Start: at 2 | ORF Stop: end of sequence |

CACCGGATCCACCATGGCCAACTCAGGCCTCCAGCTCCTGGGCTACTTCTTGGCCCTGGGTGGCTGGG
TGGGCATCATTGCTAGCACAGCCCTGCCACAGTGGAAGCAGTCTTCCTACGCAGGCGACGCCATCATC
ACTGCCGTGGGCCTCTATGAAGGGCTCTGGATGTCCTGCGCCTCCCAGAGCACTGGGCAAGTGCAGTG
CAAGCTCTACGACTCGCTGCTCGCCCTGGACGGTCACATCCAATCAGCGCGGGCCCTGATGGTGGTGG
CCGTGCTCCTGGGCTTCGTGGCCATGGTCCTCAGCGTAGTTGGCATGAAGTGTACGCGGGTGGGAGAC
AGCAACCCCATTGCCAAGGGCCGTGTTGCCATCGCCGGGGGAGCCCTCTTCATCCTGGCAGGCCTCTG
CACTTTGACTGCTGTCTCGTGGTATGCCACCCTGGTGACCCAGGAGTTCTTCAACCCAAGCACACCTG
TCAATGCCAGGTATGAATTTGGCCCAGCCCTGTTCGTGGGCTGGGCCTCAGCTGGCCTGGCCGTGCTG
GGCGGCTCCTTCCTCTGCTGCACATGCCCGGAGCCAGAGAGACCCAACAGCAGCCCACAGCCCTATCG
GCCTGGACCCTCTGCTGCTGCCCGAGAGTACGTCGTCGACGGC

NOV38b, 277685552

| | | | |
|---|---|---|---|
| Protein Sequence | SEQ ID NO: 892 | 218 aa | MW at 22694.0kD |

TGSTMANSGLQLLGYFLALGGWVGIIASTALPQWKQSSYAGDAIITAVGLYEGLWMSCASQSTGQVQC
KLYDSLLALDGHIQSARALMVVAVLLGFVAMVLSVVGMKCTRVGDSNPIAKGRVAIAGGALFILAGLC
TLTAVSWYATLVTQEFFNPSTPVNARYEFGPALFVGWASAGLAVLGGSFLCCTCPEPERPNSSPQPYR
PGPSAAAREYVVDG

| | | |
|---|---|---|
| NOV38c, 277685649 | SEQ ID NO: 893 | 583 bp |

| | | |
|---|---|---|
| DNA Sequence | ORF Start: at 2 | ORF Stop: end of sequence |

CACCGGATCCAGCACAGCCCTGCCACAGTGGAAGCAGTCTTCCTACGCAGGCGACGCCATCATCACTG
CCGTGGGCCTCTATGAAGGGCTCTGGATGTCCTGCGCCTCCCAGAGCACTGGGCAAGTGCAGTGCAAG
CTCTACGACTCGCTGCTCGCCCTGGACGGTCACATCCAATCAGCGCGGGCCCTGATGGTGGTGGCCGT
GCTCCTGGGCTTCGTGGCCATGGTCCTCAGCGTAGTTGGCATGAAGTGTACGCGGGTGGGAGACAGCA
ACCCCATTGCCAAGGGCCGTGTTGCCATCGCCGGGGGAGCCCTCTTCATCCTGGCAGGCCTCTGCACT
TTGACTGCTGTCTCGTGGTATGCCACCCTGGTGACCCAGGAGTTCTTCAACCCAAGCACACCTGTCAA
TGCCAGGTATGAATTTGGCCCAGCCCTGTTCGTGGGCTGGGCCTCAGCTGGCCTGGCCGTGCTGGGCG
GCTCCTTCCTCTGCTGCACATGCCCGGAGCCAGAGAGACCCAACAGCAGCCCACAGCCCTATCGGCCT
GGACCCTCTGCTGCTGCCCGAGAGTACGTCGTCGACGGC

NOV38c, 277685649

| | | | |
|---|---|---|---|
| Protein Sequence | SEQ ID NO: 894 | 194 aa | MW at 20246.1kD |

TGSSTALPQWKQSSYAGDAIITAVGLYEGLWMSCASQSTGQVQCKLYDSLLALDGHIQSARALMVVAV
LLGFVAMVLSVVGMKCTRVGDSNPIAKGRVAIAGGALFILAGLCTLTAVSWYATLVTQEFFNPSTPVN
ARYEFGPALFVGWASAGLAVLGGSFLCCTCPEPERPNSSPQPYRPGPSAAAREYVVDG

| | | |
|---|---|---|
| NOV38d, 277685616 | SEQ ID NO: 895 | 556 bp |

| | | |
|---|---|---|
| DNA Sequence | ORF Start: at 2 | ORF Stop: end of sequence |

CACCGGATCCTCAGGCCTCCAGCTCCTGGGCTACTTCTTGGCCCTGGGTGGCTGGGTGGGCATCATTG
CTAGCACAGCCCTGCCACAGTGGAAGCAGTCTTCCTACGCAGGCGACGCCATCATCACTGCCGTGGGC
CTCTATGAAGGGCTCTGGATGTCCTGCGCCTCCCAGAGCACTGGGCAAGTGCAGTGCAAGCTCTACGA
CTCGCTGCTCGCCCTGGACGGTCACATCCAATCAGCGCGGGCCCTGATGGTGGTGGCCGTGCTCCTGG
GCTTCGTGGCCATGGTCCTCAGCGTAGTTGGCATGAAGTGTACGCGGGTGGGAGACAGCAACCCCATT
GCCAAGGGCCGTGTTGCCATCGCCGGGGGAGCCCTCTTCATCCTGGCAGGCCTCTGCACTTTGACTGC
TGTCTCGTGGTATGCCACCCTGGTGACCCAGGAGTTCTTCAACCCAAGCACACCTGTCAATGCCAGGT
ATGAATTTGGCCCAGCCCTGTTCGTGGGCTGGGCCTCAGCTGGCCTGGCCGTGCTGGGCGGCTCCTTC
CTCGTCGACGGC

NOV38d, 277685616

| | | | |
|---|---|---|---|
| Protein Sequence | SEQ ID NO: 896 | 185 aa | MW at 19131.1kD |

TGSSGLQLLGYFLALGGWVGIIASTALPQWKQSSYAGDAIITAVGLYEGLWMSCASQSTGQVQCKLYD
SLLALDGHIQSARALMVVAVLLGFVAMVLSVVGMKCTRVGDSNPIAKGRVAIAGGALFILAGLCTLTA
VSWYATLVTQEFFNPSTPVNARYEFGPALFVGWASAGLAVLGGSFLVDG

A ClustalW comparison of the above protein sequences yields the following sequence alignment shown in Table 38B.

TABLE 38B
Comparison of the NOV38 protein sequences.

| | |
|---|---|
| NOV38a | ----MANSGLQLLGYFLALGGWVGIIASTALPQWKQSSYAGDAIITAVGLYEGLWMSCAS |
| NOV38b | TGSTMANSGLQLLGYFLALGGWVGIIASTALPQWKQSSYAGDAIITAVGLYEGLWMSCAS |
| NOV38c | ------------------------TGSSTALPQWKQSSYAGDAIITAVGLYEGLWMSCAS |
| NOV38d | ----TGSSGLQLLGYFLALGGWVGIIASTALPQWKQSSYAGDAIITAVGLYEGLWMSCAS |
| NOV38a | QSTGQVQCKLYDSLLALDGRPQAARALMVVAVLLGFVAMVLSVVGMKCTRVGDSNPIAKG |
| NOV38b | QSTGQVQCKLYDSLLALDGHIQSARALMVVAVLLGFVAMVLSVVGMKCTRVGDSNPIAKG |
| NOV38c | QSTGQVQCKLYDSLLALDGHIQSARALMVVAVLLGFVAMVLSVVGMKCTRVGDSNPIAKG |
| NOV38d | QSTGQVQCKLYDSLLALDGHIQSARALMVVAVLLGFVAMVLSVVGMKCTRVGDSNPIAKG |
| NOV38a | RVAIAGGALFILAGLCTLTAVSWYATLVTQEFFNP--------EFGPALFVGWASAGLAV |
| NOV38b | RVAIAGGALFILAGLCTLTAVSWYATLVTQEFFNPSTPVNARYEFGPALFVGWASAGLAV |
| NOV38c | RVAIAGGALFILGGLCTLTAVSWYATLVTQEFFNPSTPVNARYEFGPALFVGWASAGLAV |
| NOV38d | RVAIAGGALFILAGLCTLTAVSWYATLVTQEFFNPSTPVNARYEFGPALFVGWASAGLAV |
| NOV38a | LGGSFLCCTCPEPERPNSSPQPYRPGPSAAAREYV--- |
| NOV38b | LGGSFLCCTCPEPERPNSSPQPYRPGPSAAAREYVVDG |
| NOV38c | LGGSFLCCTCPEPERPNSSPQPYRPGPSAAAREYVVDG |
| NOV38d | LGGSFLVDG----------------------------- |
| NOV38a | (SEQ ID NO: 890) |
| NOV38b | (SEQ ID NO: 892) |
| NOV38c | (SEQ ID NO: 894) |
| NOV38d | (SEQ ID NO: 896) |

Further analysis of the NOV38a protein yielded the following properties shown in Table 38C.

TABLE 38C
Protein Sequence Properties NOV38a

| | |
|---|---|
| SignalP analysis: | Cleavage site between residues 24 and 25 |
| PSORT II analysis: | |

| PSG: | a new signal peptide prediction method | |
|---|---|---|
| | N-region: | length 0; pos. chg 0; neg. chg 0 |
| | H-region: | length 30; peak value 9.10 |
| | PSG score: | 4.70 |

| | |
|---|---|
| GvH: | von Heijne's method for signal seq. recognition |
| | GvH score (threshold: −2.1): −4.10 |
| | possible cleavage site: between 17 and 18 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 4 | | |
| | INTEGRAL | Likelihood = | Transmembrane 8-24 |
| | | −1.75 | |
| | INTEGRAL | Likelihood = | Transmembrane 84-100 |
| | | −12.15 | |
| | INTEGRAL | Likelihood = | Transmembrane 118-134 |
| | | −6.42 | |
| | INTEGRAL | Likelihood = | Transmembrane 153-169 |
| | | −0.64 | |

| | |
|---|---|
| PERIPHERAL | Likelihood = 2.28 (at 39) |

| | |
|---|---|
| | ALOM score: −12.15 (number of TMSs: 4) |
| MTOP: | Prediction of membrane topology (Hartmann et al.) |
| | Center position for calculation: 15 |
| | Charge difference: −1.0 C(0.0)-N(1.0) |
| | N >= C: N-terminal side will be inside |

>>> membrane topology: type 3a

| MITDISC: | discrimination of mitochondrial targeting seq | | | |
|---|---|---|---|---|
| | R content: | 0 | Hyd Moment(75): | 4.90 |
| | Hyd Moment(95): | 7.23 | G content: | 6 |
| | D/E content: | 1 | S/T content: | 5 |

| | |
|---|---|
| | Score: −6.38 |
| Gavel: | prediction of cleavage sites for mitochondrial preseq |
| | cleavage site motif not found |

NUCDISC: discrimination of nuclear localization signals

| | |
|---|---|
| | pat4: none |
| | pat7: none |
| | bipartite: none |
| | content of basic residues: 5.4% |
| | NLS Score: −0.47 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| | total: 0 residues |

Final Results (k = 9/23):

66.7%: endoplasmic reticulum
33.3%: mitochondrial

>> prediction for CG56594-01 is end (k = 9)

A search of the NOV38a protein against the Geneseq database, a proprietary database that contains sequences published in patents and patent publication, yielded several homologous proteins shown in Table 38D.

TABLE 38D
Geneseq Results for NOV38a

| | | NOV38a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABG27980 | Novel human diagnostic protein | 1 . . . 203 | 200/211 (94%) | e−111 |
| | #27971 - Homo sapiens, 254 aa. | 44 . . . 254 | 201/211 (94%) | |
| | [WO200175067-A2, 11-OCT-2001 ] | | | |
| AAO20545 | Human Claudin-19 protein - Homo | 1 . . . 203 | 197/211 (93%) | e−109 |
| | sapiens, 211 aa. [WO200214499-A2, | 1 . . . 211 | 198/211 (93%) | |
| | 21-FEB-2002] | | | |
| AAO20546 | Murine Claudin-19 protein (partial | 11 . . . 195 | 172/193 (89%) | 1e−94 |
| | sequence) - Mus sp, 193 aa. | 1 . . . 193 | 175/193 (90%) | |
| | [WO200214499-A2, 21-FEB-2002] | | | |
| AAU96166 | Human secreted protein, SEQ ID No | 84 . . . 203 | 120/128 (93%) | 2e−63 |
| | 68 - Homo sapiens, 128 aa. | 1 . . . 128 | 120/128 (93%) | |
| | [WO200224721-A1, 28-MAR-2002] | | | |
| ABG64826 | Human albumin fusion protein #1501 - | 84 . . . 203 | 120/128 (93%) | 2e−63 |
| | Homo sapiens, 128 aa. | 1 . . . 128 | 120/128 (93%) | |
| | [WO200177137-A1, 18-OCT-2001] | | | |

In a BLAST search of public sequence databases, the NOV38a protein was found to have homology to the proteins shown in the BLASTP data in Table 38E.

TABLE 38E
Public BLASTP Results for NOV38a

| | | NOV38a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8N6F1 | Claudin-19 - Homo sapiens (Human), | 1 . . . 203 | 200/211 (94%) | e−111 |
| | 211 aa. | 1 . . . 211 | 201/211 (94%) | |
| Q9ET38 | Claudin-19 - Mus musculus (Mouse), | 1 . . . 203 | 191/211 (90%) | e−105 |
| | 211 aa. | 1 . . . 211 | 195/211 (91%) | |
| O95832 | Claudin-1 (Senescence-associated | 1 . . . 203 | 115/212 (54%) | 1e−62 |
| | epithelial membrane protein) - Homo | 1 . . . 211 | 151/212 (70%) | |
| | sapiens (Human), 211 aa. | | | |
| AAP35475 | Claudin 7 - Homo sapiens (Human), | 1 . . . 203 | 112/211 (53%) | 3e−62 |
| | 211 aa. | 1 . . . 211 | 149/211 (70%) | |
| Q8AVG4 | Similar to claudin 1 - Xenopus laevis | 1 . . . 203 | 112/211 (53%) | 6e−62 |
| | (African clawed frog), 211 aa. | 1 . . . 211 | 142/211 (67%) | |

PFam analysis predicts that the NOV38a protein contains the domains shown in the Table 38F.

TABLE 38F
Domain Analysis of NOV38a

| | | Identities/ | | |
|---|---|---|---|---|
| | | Similarities | | |
| | NOV38a Match | for the | | Expect |
| Pfam Domain | Region | Matched Region | | Value |
| PMP22_Claudin | 4 . . . 174 | 61/188 | (32%) | 1.7e−56 |
| | | 142/188 | (76%) | |

### Example 39

The NOV39 clone was analyzed, and the nucleotide and encoded polypeptide sequences are shown in Table 39A.

TABLE 39A
NOV39 Sequence Analysis

| | | |
|---|---|---|
| NOV39a, CG56653-08 | SEQ ID NO: 897 | 1000 bp |

| | | |
|---|---|---|
| DNA Sequence | ORF Start: ATG at 2 | ORF Stop: TAG at 980 |

CATGGAGCTGAGTGGAGCCACCATGGCCCGGGGGCTCGCTGTCCTGCTAGTCTTGTTCCTGCATATCA
AGAACCTGCCTGCCCAGGCTGCGGACACATGTCCAGAGGTGAAGGTGGTGGGCCTGGAGGGCTCTGAC
AAGCTCACCATTCTCCGAGGCTGCCCGGGGCTGCCCGGGGCCCCAGGGCCAAAGGGAGAGGCAGGTGT
CATTGGAGAGAGAGGAGAACGCGGTCTCCCTGGAGCCCCTGGAAAGGCAGGACCAGTGGGGCCCAAGG
GAGACCGAGGAGAGAAGGGGATGCGTGGAGAGAAAGGAGACGCTGGGCAGTCTCAGTCGTGTGCGACA
GGCCCACGCAACTGCAAGGACCTGCTAGACCGGGGGTATTTCCTGAGCGGCTGGCACAATATCTACCT
GCCCGACTGCCGGCCCCTGACTGTGCTCTGTGACATGGACACGGACGGAGGGGGCTGGACCGTTTTCC
AGCGGAGGATGGATGGCTCTGTGGACTTCTATCGGGACTGGGCCGCATACAAGCAGGGCTTCGGCAGT
CAGCTGGGGGAGTTCTGGCTGGGGAACGACAACATCCACGCCCTGACTGCCCAGGGAAGCAGCGAGCT
CCGTGTAGACCTGGTGGACTTTGAGGGCAACCACCAGTTTGCTAAGTACAAATCATTCAAGGTGGCTG
ACGAGGCAGAGAAGTACAAGCTGGTACTGGGAGCCTTTGTCGGGGGCAGTGCGGGTAATTCTCTAACG
GGCCACAACAACAACTTCTTCTCCACCAAAGACCAAGACAATGATGTGAGTTCTTCGAATTGTGCTGA
GAAGTTCCAGGGAGCCTGGTGGTACGCCGACTGTCATGCTTCGAGCCTCAATGGTCTCTACCTCATGG
GTGTCAGAGATGAAGGTGCGGCCCGCCTAGACGGGCCAGGACCCCTAA

NOV39a, CG56653-08

| | | | |
|---|---|---|---|
| Protein Sequence | SEQ ID NO: 898 | 326 aa | MW at 35064.1kD |

MELSGATMARGLAVLLVLFLHIKNLPAQAADTCPEVKVVGLEGSDKLTILRGCPGLPGAPGPKGEAGV
IGERGERGLPGAPGKAGPVGPKGDRGEKGMRGEKGDAGQSQSCATGPRNCKDLLDRGYFLSGWHNIYL
PDCRPLTVLCDMDTDGGGWTVFQRRMDGSVDFYRDWAAYKQGFGSQLGEFWLGNDNIHALTAQGSSEL
RVDLVDFEGNHQFAKYKSFKVADEAEKYKLVLGAFVGGSAGNSLTGHNNNFFSTKDQDNDVSSSNCAE
KFQGAWWYADCHASSLNGLYLMGPHESYANGINWSAAKGYKYSYKVSEMKVRPA

| | | |
|---|---|---|
| NOV39b, 214374274 | SEQ ID NO: 899 | 651 bp |

| | | |
|---|---|---|
| DNA Sequence | ORF Start: at 1 | ORF Stop: end of sequence |

GGATCCGGCCCACGCAACTGCAAGGACCTGCTAGACCGGGGGTATTTCCTGAGCGGCTGGACACCAT
CTACCTGCCCGACTGCCGGCCCCTGACTGTGCTCTGTGACATGGACACGGACGGAGGGGGCTGGACCG
TTTTCCAGCGGAGGATGGATGGCTCTGTGGACTTCTATCGGGACTGGGCCGCATACAAGCAGGGCTTC
GGCAGTCAGCTGGGGGAGTTCTGGCTGGGGAACGACAACATCCACGCCCTGACTGCCCAGGGAAGCAG
CGAGCTCCGTGTAGACCTGGTGGACTTTGAGGGCAACCACCAGTTTGCTAAGTACAAATCATTCAAGG
TGGCTGACGAGGCAGAGAAGTACAAGCTGGTACTGGGAACCTTTGTCGGGGGCAGTGCGGGTAATTCT
CTAACGGGCCACAACAACAACTTCTTCTCCACCAAAGACCAAGACAATGATGTGAGTTCTTCGAATTG
TGCTGAGAAGTTCCAGGGAGCCTGGTGGTACGCCGACTGTCATGCTTCAAACCTCAATGGTCTCTACC
TCATGGGACCCCATGAGAGCTATGCCAATGGTATCAACTGGAGTGCGGCGAAGGGGTACAAATATAGC
TACAAGGTGTCAGAGATGAAGGTGCGGCCCGCCCTCGAG

NOV39b, 214374274

| | | | |
|---|---|---|---|
| Protein Sequence | SEQ ID NO: 900 | 217 bp | MW at 24272.7kD |

GSGPRNCKDLLDRGYFLSGWHTIYLPDCRPLTVLCDMDTDGGGWTVFQRRMDGSVDFYRDWAAYKQGF
GSQLGEFWLGNDNIHALTAQGSSELRVDLVDFEGNIIQFAKYKSFKVADEAEKYKLVLGTFVGGSAGNS
LTGHNNNFFSTKDQDNDVSSSNCAEKFQGAWWYADCHASNLNGLYLMGPHESYANGINWSAAKGYKYS
YKVSEMKVRPALE

| | | |
|---|---|---|
| NOV39c, 214374256 | SEQ ID NO: 901 | 651 bp |

| | | |
|---|---|---|
| DNA Sequence | ORF Start: at 1 | ORF Stop: end of sequence |

GGATCCGGCCCACGCAACTGCAAGGACCTGCTAGACCGGGGGTATTTCCTGAGCGGCTGGCACACCAT
CTACCTGCCCGACTGCCGGCCCCTGACTGTGCTCTGTGACATGGACACGGACGGAGGGGGCTGGACCG
TTTTCCAGCGGAGGATGGATGGCTCTGTGGACTTCTATCGGGACTGGGCCGCATACAAGCAGGGCTTC
GGCAGTCAGCTGGGGGAGTTCTGGCTGGGGAATGACAACATCCACGCCCTGACTGCCCAGGGAAGCAG
CGAGCTCCGTGTAGACCTGGTGGACTTTGAGGGCAACCACCAGTTTGCTAAGTACAAATCATTCAAGG
TGGCTGACGAGGCAGAGAAGTACAAGCTGGTACTGGGAGCCTTTGTCGGGGGCAGTGCGGGTAATTCT
CTAACGGGCCACAACAACAACTTCTTCTCCACCAAAGACCAAGACAATGATGTGAGTTCTTCGAATTG
TGCTGAGAAGTTCCAAGGAGCCTGGTGGTACGCCGACTGTCATGCTTCAAACCTCAATGGTCTCTACC
TCATGGGACCCCATGAGAGCTATGCCAATGGTATCAACTGGAGTGCGGCGAAGGGGTACAAATATAGC
TACAAGGTGTCAGAGATGAAGGTGCGGCCCGCCCTCGAG

NOV39c, 214374256

| | | | |
|---|---|---|---|
| Protein Sequence | SEQ ID NO: 902 | 217 aa | MW at 24242.6kD |

GSGPRNCKDLLDRGYFLSGWHTIYLPDCRPLTVLCDMDTDGGGWTVFQRPMDGSVDFYRDWAAYKQGF
LTGHNNNFFSTKDQDNDVSSSNCAEKFQGAWWYADCHASNLNGLYLMGPHESYANGINWSAAKGYKYS
YKVSEMKVRPALE

| | | |
|---|---|---|
| NOV39d, 214374252 | SEQ ID NO: 903 | 651 bp |

| | | |
|---|---|---|
| DNA Sequence | ORF Start: at 1 | ORF Stop: end of sequence |

GGATCCGGCCCACGCAACTGCAAGGACCTGCTAGACCGGGGGTATTTCCTGAGCGGCTGGCACACCAT
CTACCTGCCCGACTGCCGGCCCCTGACTGTGCTCTGTGACATGGACACGGACGGAGGGGGCTGGACCG
GTTTCCAGCGGAGGATGGATGGCTCTGTGGACTTCTATCGGGACTGGGCCGCATACAAGCAGGGCTTC
GGCAGTCAGCTGGGGGAGTTCTGGCTGGGGAACGACAACATCCACGCCCTGACTGCCCAGGGAAGCAG
CGAGCTCCGTGTAGACCTGGTGGACTTTGAGGGCAACCACCAGTTTGCTAAGTACAAATCATTCAAGG
TGGCTGACGAGGCAGAGAAGTACAAGCTGGTACTGGGAGCCTTTGTCGGGGGCAGTGCGGGTAATTCT
CTAACGGGCCACAACAACAACTTCTTCTCCACCAAAGACCAAGACAATGATGTGAGTTCTTCGAATTG
TCATGGGACCCCATGAGAGCTATGCCAATGGTATCAACTGGAGTGCGGCGAAGGGGTACAAATATAGC
TACAAGGTGTCAGAGATGAAGGTGCGGCCCGCCCTCGAG

NOV39d, 214374252

| | | | |
|---|---|---|---|
| Protein Sequence | SEQ ID NO: 904 | 217 aa | MW at 24200.6kD |

GSGPRNCKDLLDRGYFLSGWHTIYLPDCRPLTVLCDMDTDGGGWTGFQRRMDGSVDFYRDWAAYKQGF
GSQLGEFWLGNDNIHALTAQGSSELRVDLVDFEGNHQFAKYKSFKVADEAEKYKLVLGAFVGGSAGNS
LTGHNNNFFSTKDQDNDVSSSNCAEKFQGAWWYADCHASNLNGLYLMGPHESYANGINWSAAKGYKYS
YKVSEMKVRPALE

| | | |
|---|---|---|
| NOV39e, 214458492 | SEQ ID NO: 905 | 651 bp |

| | | |
|---|---|---|
| DNA Sequence | ORF Start: at 1 | ORF Stop: end of sequence |

GGATCCGGCCCACGCAACTGCAAGGACCTGCTAGACCGGGGGTATTTCCTGAGCGGCTGGCACACCAT
CTACCTGCCCGACTGCCGGCCCCTGACTGTGCTCTGTGACATGGACACGGACGGAGGGGGTTGGACCG
TTTTCCAGCGGAGGATGGATGGCTCTGTGGACTTCTATCGGGACTGGGCCGCATACAAGCAGGGCTTC
GGCAGTCGGCTGGGGGAGTTCTGGCTGGGGAACGACAACATCCACGCCCTGACTGCCCAGGGAAGCAG
CGAGCTCCGTGTAGACCTGGTGGACTTTGAGGGCAACCACCAGTTTGCTAAGTACAAATCATTCAAGG
TGGCTGACGAGGCAGAGAAGTACAAGCTGGTACTGGGAGCCTTTGTCGGGGGCAGTGCGGGTAATTCT
CTAACGGGCCACAACAACAACTTCTTCTCCACCAAAGACCAAGACAATGATGTGAGTTCTTCGAATTG
TGCTGAGAAGTTCCAGGGAGCCTGGTGGTACGCCGACTGTCATGCTTCAAACCTCAATGGTCTCTACC
TCATGGGACCCCATGAGAGCTATGCCAATGGTATCAACTGGAGTGCGGCGAAGGGGTACAAATATAGC
TACAAGGTGTCAGAGATGAAGGTGCGGCCCGCCCTCGAG

NOV39e, 214458492

| | | | |
|---|---|---|---|
| Protein Sequence | SEQ ID NO: 906 | 217 aa | MW at 24270.7kD |

GSRLGEFWLGNDNIHALTAQGSSELRVDLVDFEGNHQFAKYKSFKVADEAEKYKLVLGAFVGGSAGNS
LTGHNNNFFSTKDQDNDVSSSNCAEKFQGAWWYADCHASNLNGLYLMGPHESYANGINWSAAKGYKYS
YKVSEMKVRPALE

| | | |
|---|---|---|
| NOV39f, 214458488 | SEQ ID NO: 907 | 651 bp |

| | | |
|---|---|---|
| DNA Sequence | ORF Start: at 1 | ORF Stop: end of sequence |

GGATCCGGCCCACGCAACTGCAAGGACCTGCTAGACCGGGGGTATTTCCTGAGCGGCTGGCACACCAT
CTACCTGCCCGACTGCCGGCCCCTGACTGTGCTCTGTGACATGGACACGGACGGAGGGGGCTGGACCG
TTTTCCAGCGGAGGATGGATGGCTCTGTGGACTTCTATCGGGACTGGGCCGCATACAAGCAGGGCTTC
GGCAGTCAGCTGGGGGAGTTCTGGCTGGGGAATGACGACATCCACGCCCTGACTGCCCAGGGAAGCAG
CGAGCTCCGTGTAGACCTGGTGGACTTTGAGGGCAACCACCAGTTTGCTAAGTACAAATCATTCAAGG
TGGCTGACGAGGCAGAGAAGTACAAGCTGGTACTGGGAGCCTTTGTCGGGGGCAGTGCGGGTAATTCT
CTAACGGGCCACAACAACAACTTCTTCTCCACCAAAGACCAAGACAATGATGTGAGTTCTTCGAATTG
TGCTGAGAAGTTCCAAGGAGCCTGGTGGTACGCCGACTGTCATGCTTCAAACCTCAATGGTCTCTACC
TCATGGGACCCCATGAGAGCTATGCCAATGGTATCAACTGGAGTGCGGCGAAGGGGTACAAATATAGC
TACAAGGTGTCAGAGATGAAGGTGCGGCCCGCCCTCGAG

NOV39f, 214458488

| | | | |
|---|---|---|---|
| Protein Sequence | SEQ ID NO: 908 | 217 aa | MW at 24243.6kD |

GSGPRNCKDLLDRGYFLSGWHTIYLPDCRPLTVLCDMDTDGGGWTVFQRRMDGSVDFYRDWAAYKQGF
GSQLGEFWLGNDDIHALTAQGSSELRVDLVDFEGNHQFAKYKSFKVADEAEKYKLVLGAFVGGSAGNS
LTGHNNNFFSTKDQDNDVSSSNCAEKFQGAWWYADCHASNLNGLYLMGPHESYANGINWSAAKGYKYS
YKVSEMKVRPALE

| | | |
|---|---|---|
| NOV39g, 214374236 | SEQ ID NO: 909 | 651 bp |

| | | |
|---|---|---|
| DNA Sequence | ORF Start: at 1 | ORF Stop: end of sequence |

GGATCCGGCCCACGCAACTGCAAGGACCTGCTAGACCGAGGGCACTTCCTGAGCGGCTGGCACACCAT
CTACCTGCCCGACTGCCGGCCCCTGACTGTGCTCTGTGACATGGACACGGACGGAGGGGGCTGGACCG
TTTTCCAGCGGAGGATGGATGGCTCTGTGGACTTCTATCGGGACTGGGCCGCATACAAGCAGGGCTTC
GGCAGTCAGCTGGGGGAGTTCTGGCTGGGGAACGACAACATCCACGCCCTGACTGCCCAGGGAAGCAG
CGAGCTCCGTGTAGACCTGGTGGACTTTGAGGGCAACCACCAGTTTGCTAAGTACAAATCATTCAAGG
TGGCTGACGAGGCAGAGAAGTACAAGCTGGTACTGGGAGCCTTTGTCGGGGGCAGTGCGGGTAATTCT
CTAACGGGCCACAACAACAACTTCTTCTCCACCAAAGACCAAGACAATGATGTGAGTTCTTCGAATTG
TGCTGAGAAGTTCCAAGGAGCCTGGTGGTACGCCGACTGTCATGCTTCAAACCTCAATGGTCTCTACC
TCATGGGACCCCATGAGAGCTATGCCAATGGTATCAACTGGAGTGCGGCGAAGGGGTACAAATATAGC
TACAAGGTGTCAGAGATGAAGGTGCGGCCCGCCCTCGAG

NOV39g 214374236

| | | | |
|---|---|---|---|
| Protein Sequence | SEQ ID NO: 910 | 217 aa | MW at 24216.6kD |

GSGPRNCKDLLDRGHFLSGWHTIYLPDCRPLTVLCDMDTDGGGWTVFQRRMDGSVDFYRDWAAYKQGF
GSQLGEFWLGNDNIHALTAQGSSELRVDLVDFEGNHQFAKYKSFKVADEAEKYKLVLGAFVGGSAGNS
LTGHNNNFFSTKDQDNDVSSSNCAEKFQGAWWYADCHASNLNGLYLMGPHESYANGINWSAAKGYKYS
YKVSEMKVRPALE

| | | |
|---|---|---|
| NOV39h, CG56653-01 | SEQ ID NO: 911 | 1187 bp |

| | | |
|---|---|---|
| DNA Sequence | ORF Start: ATG at 16 | ORF Stop: TAG at 973 |

CTGAGTGGAGCCACCATGGCCCGGGGGCTCGCTGTCCTGCTAGTCTTGTTCCTGCATATCAAGAACCT
GCCTGCCCAGGCTGCGGACACATGTCCAGAGGTGAAGGTGGTGGGCCTGGAGGGCTCTGACAAGCTCA
CCATTCTCCGAGGCTGCCCGGGGCTGCCCGGGGCCCCAGGGCCAAAGGGAGAGGCAGGTGTCATTGGA
GAGAGAGGAGAACGCGGTCTCCCTGGAGCCCCTGGAAAGGCAGGACCAGTGGGGCCCAAAGGAGACCG
AGGAGAGAAGGGGATGCGTGGAGAGAAAGGAGACGCTGGGCAGTCTCAGTCGTGTGCGACAGGCCCAC
GCAACTGCAAGGACCTGCTAGACCGGGGGTATTTCCTGAGCGGCTGGCACACCATCTACCTGCCCGAC
TGCCGGCCCCTGACTGTGCTCTGTGACATGGACACGGACGGAGGGGGCTGGACCGTTTTCCAGCGGAG
GATGGATGGCTCTGTGGACTTCTATCGGGACTGGGCCGCATACAAGCAGGGCTTCGGCAGTCAGCTGG
GGGAGTTCTGGCTGGGGAACGACAACATCCACGCCCTGACTGCCCAGGGAAGCAGCGAGCTCCGTGTA
GACCTGGTGGACTTTGAGGGCAACCACCAGTTTGCTAAGTACAAATCATTCAAGGTGGCTGACGAGGC
AGAGAAGTACAAGCTGGTACTGGGAGCCTTTGTCGGGGGCAGTGCGGGTAATTCTCTAACGGGCCACA
ACAACAACTTCTTCTCCACCAAAGACCAAGACAATGATGTGAGTTCTTCGAATTGTGCTGAGAAGTTC
CAGGGAGCCTGGTGGTACGCCGACTGTCATGCTTCAAACCTCAATGGTCTCTACCTCATGGGACCCCA
TGAGAGCTATGCCAATGGTATCAACTGGAGTGCGGCGAAGGGGTACAAATATAGCTACAAGGTGTCAG
AGATGAAGGTGCGGCCCGCCTAGACGGGCCAGGACCCCTCCACATGCACCTGCTAGTGGGGAGGCCAC
ACCCACAAGCGCTGCGTCGTGGAAGTCACCCCATTTCCCCAGCCAGACACACTCCCATGACGCCCACA
GCTGCCCCTTTGCCCCCAGCTCAGTCAAGCCGCCACATGCCCACAACCTCACCAGAGGGAGAATTATG
TTTCTAAATATGTTTACTTTGGGACAGAAAA

NOV39h, CG56653-01

| | | | |
|---|---|---|---|
| Protein Sequence | SEQ ID NO: 912 | 319 aa | MW at 34388.3kD |

MARGLAVLLVLFLHIKNLPAQAADTCPEVKVVGLEGSDKLTILRGCPGLPGAPGPKGEAGVIGERGER
GLPGAPGKAGPVGPKGDRGEKGMRGEKGDAGQSQSCATGPRNCKDLLDRGYFLSGWHTIYLPDCRPLT
VLCDMDTDGGGWTVFQRRMDGSVDFYRDWAAYKQGFGSQLGEFWLGNDNIHALTAOGSSELRVDLVDF
EGNHQFAKYKSFKVADEAEKYKLVLGAFVGGSAGNSLTGHNNNFFSTKDQDNDVSSSNCAEKFQGAWW
YADCHASNLNGLYLMGPHESYANGINWSAAKGYKYSYKVSEMKVRPA

| | | |
|---|---|---|
| NOV39i, CG56653-02 | SEQ ID NO: 913 | 1332 bp |

| | | |
|---|---|---|
| DNA Sequence | ORF Start: ATG at 183 | ORF Stop: TAG at 1107 |

TTTTAGGTCTGTTTGTCGTAGGCAGATGGAGCTTGTTATAATTATGCCTCATAGGGATAGTACAAGGA
AGGGGTAGGCTATGTGTTTTGTCAGGGAGTTGAGAAACTGTGGCACAAGGCGAGAGCTGGTTTCCTCT
GCCCTGTTAGAGCTGGGGGACTCTTCAGAGTCAAAGGCCAGAGAGCATGGAGCTGAGTGGAGCCACCA
TGGCCCGGGGGCTCGCTGTCCTGCTAGTCTTGTTCCTGCATATCAAGAACCTGCCTGCCCAGGCTGCG
GACACATGTCCAGAGGTGAAGGTGGTGGGCCTGGAGGGCTCTGGCAAGCTCACCATTCTCCGAGGCTG
CCCGGGGCTGCCCGGGGCCCCAGGGCCAAAGGGAGAGGCAGGTGTCATTGGAGAGAGAGGAGACCGAG
GAGAGAAGGGGATGCGTGGAGAGAAAGGAGACGCTGGGCAGTCTCAGTCGTGTGCGACAGGCCCACGC
AACTGCAAGGACCTGCTAGACCGGGGGTATTTCCTGAGCGGCTGGCACACCATCTACCTGCCCGACTG
CCGGCCCCTGACTGTGCTCTGTGACATGGACACGGACGGAGGGGGCTGGACCGTTTTCCAGCGGAGGA
TGGATGGCTCTGTGGACTTCTATCGGGACTGGGCCGCATACAAGCAGGGCTTCGGCAGTCAGCTGGGG
GAGTTCTGGCTGGGGAATGACAACATCCACGCCCTGACTGCCCAGGGAAGCAGCGAGCTCCGTGTAGA
CCTGGTGGACTTTGAGGGCAACCACCAGTTTGCTAAGTACAAATCATTCAAGGTGGCTGACGAGGCAG
AGAAGTACAAGCTGGTACTGGGAGCCTTTGTCGGGGGCAGTGCGGGTAATTCTCTAACGGGCCACAAC
AACAACTTCTTCTCCACCAAAGACCAAGACAATGATGTGAGTTCTTCGAATTGTGCTGAGAAGTTCCA
AGGAGCCTGGTGGTACGCCGACTGTCATGCTTCAAACCTCAATGGTCTCTACCTCATGGGACCCCATG
AGAGCTATGCCAATGGTATCAACTGGAGTGCGGCGAAGGGGTACAAATATAGCTACAAGGTGTCAGAG
ATGAAGGTGCGGCCCGCCTAGACGGGCCAGGACCCCTCCACATGCACCTGCTAGTGGGGAGGCCACAC
CCACAAGCGCTGCGTCGTGGAAGTCACCCCATTTCCCCAGCCAGACACACTCCCATGACGCCCACAGC
TCTAATATGTTTACTTTTGGGGACAGAAAAAAAAAAAAA

NOV39i, CG56653-02

| | | | |
|---|---|---|---|
| Protein Sequence | SEQ ID NO: 914 | 308 aa | MW at 33393.2kD |

MELSGATMARGLAVLLVLFLHIKNLPAQAADTCPEVKVVGLEGSGKLTILRGCPGLPGAPGPKGEAGV
IGERGDRGEKGNRGEKGDAGQSQSCATGPRNCKDLLDRGYFLSGWHTIYLPDCRPLTVLCDMDTDGGG
WTVFQRRNDGSVDFYRDWAAYKQGFGSQLGEFWLGNDNIHALTAQGSSELRVDLVDFEGNHQFAKYKS
FKVADEAEKYKLVLGAFVGGSAGNSLTGHNNNFFSTKDQDNDVSSSNCAEKFQGAWWYADCHASNLNG
LYLMGPHESYANGINWSAAKGYKYSYKVSEMKVRPA

| | | |
|---|---|---|
| NOV39j, CG56653-03 | SEQ ID NO: 915 | 728 bp |

| | | |
|---|---|---|
| DNA Sequence | ORF Start: at 1 | ORF Stop: TAG at 574 |

CTGCATATCAAGAACCTGCCTGCCCAGGCTGCGGACACATGTCCAGAGGTGAAGGTGGTGGGCCTGGA
GGGCTCTGACAAGCTCACCATTCTCCGAGGCTGCCCGGGGCTGCCCGGGGCCCCAGGGCCAAAGGGAG
AGGCAGGTGTCATTGGAGAGAGAGGAGAACGCGGTCTCCCTGGAGCCCCTGGAAAGGCAGGACCAGTG
GGGCCCAAAGGAGACCGAGGAGAGAAGGGGATGCGTGGAGAGAAAGGAGACGCTGGGCAGTCTCAGTC
GTGTGCGACAGGCCCACGCAACTGCAAGGACCTGCTAGACCGGGGGTATTTCCTGAGCGGCTGGCACA
CCATCTACCTGCCCGACTGCCGGCCCCTGACTGTGCTCTGTGACATGGACACGGACGGAGGGGGCTGG
ACCGTTTTCCAGGGAGCCTGGTGGTACGCCGACTGTCATGCTTCAAACCTCAATGGTCTCTACCTCAT
GGGACCCCATGAGAGCTATGCCAATGGTATCAACTGGAGTGCGGCGAAGGGGTACAAATATAGCTACA
GGAGGCCACACCCACAAGCGCTGCGTCGTGGAAGTCACCCCATTTCCCCAGCCAGACACACTCCCATG
ACGCCCACAGCTGCCCCTTTGCCCCCAGCTCAGTCAAGCCGCCACATG

NOV39j, CG56653-03

| | | | |
|---|---|---|---|
| Protein Sequence | SEQ ID NO: 916 | 191 aa | MW at 20220.7kD |

LHIKNLPAQAADTCPEVKVVGLEGSDKLTILRGCPGLPGAPGPKGEAGVIGERGERGLPGAPGKAGPV
GPKGDRGEKGMRGEKGDAGQSQSCATGPRNCKDLLDRGYFLSGWHTIYLPDCRPLTVLCDMDTDGGGW
TVFQGAWWYADCHASNLNGLYLMGPHESYANGINWSAAKGYKYSYKVSEMKVRPA

| | | |
|---|---|---|
| NOV39k, CG56653-04 | SEQ ID NO: 917 | 1104 bp |

| | | |
|---|---|---|
| DNA Sequence | ORF Start: ATG at 16 | ORF Stop: TAG at 883 |

CTGAGTGGAGCCACCATGGCCCGGGGGCTCGCTGTCCTGCTAGTCTTGTTCCTGCATATCACGAACCT
GCCTGCCCAGGCTGCGGACACATGTCCAGAGGTGAAGGTGGTGGGCCTGGAGGGCTCTGACAAGCTCA
CCATTCTCCGAGGCTGCCCGGGGCTGCCCGGGGCCCCAGGACCAAAGGGAGAGGCAGGTGTCATTGGA
GAGAAAGGAGACGCTGGGCAGTCTCAGTCGTGTGCGACAGGCCCACGCAACTGCAAGGACCTGCTAGA
CCGAGGGTATTTCCTGAGCGGCTGGCACACCATCTACCTGCCCGACTGCCGGCCCCTTACTGTGCTCT
GTGACATGGATACGGACGGAGGGGGCTGGACCGTTTTCCAGCGGAGGATGGATGGCTCTGTGGACTTC
TATCGGGACTGGGCCGCATACAAGCAGGGCTTCGGCAGTCAGCTGGGGGAGTTCTGGCTGGGGAATGA
CAACATCCACGCCCTGACTGCCCAGGGAAGCAGCGAGCTCCGTGTAGACCTGGTGGACTTTGAGGGCA
ACCACCAGTTTGCTAAGTACAAATCATTCAAGGTGGCTGACGAGGCAGAGAAGTACAAGCTGGTACTG
GGAGCCTTTGTCGGGGGCAGTGCGGGTAATTCTCTAACGGGCCACAACAACAACTTCTTCTCCACCAA
AGACCAAGACAATGATGTGAGTTCTTCGAATTGTGCTGAGAAGTTCCAGGGAGCCTGGTGGTACGCCG
ACTGTCATGCTTCAAACCTCAATGGTCTCTACCTCATGGGACCCCATGAGAGCTATGCCAATGGTATC
AACTGGAGTGCGGCGAAGGGGTACAAATATAGCTACAAGGTGTCAGAGATGAAGGTGCGGCCCGCCTA
AAGTCACCCCATTTCCCCAGCCAGACACACTCCCATGACGCCCACAGCTGCCCCTTTGCCCCCAGCTC
AGTCAAGCCGCCACATGCCCACAACCTCACCAGAGGGAGAATTATGTTTCTAAATATGTTTACTTTGG
GACAGAAAAAAAAAAA

NOV39k, CG56653-04

| | | | |
|---|---|---|---|
| Protein Sequence | SEQ ID NO: 918 | 289 aa | MW at 31404.9kD |

MARGLAVLLVLFLHITNLPAQAADTCPEVKVVGLEGSDKLTILRGCPGLPGAPGPKGEAGVIGEKGDA
AYKQGFGSQLGEFWLGNDNIHALTAQGSSELRVDLVDFEGHQFAKYKSFKVADEAEKYKLVLGAFVG
GSAGNSLTGHNNNFFSTKDQDNDVSSSNCAEKFQGAWWYADCHASNLNGLYLMGPHESYANGINWSAA
KGYKYSYKVSEMKVRPA

| | | |
|---|---|---|
| NOV39l, CG56653-05 | SEQ ID NO: 919 | 900 bp |

| | | |
|---|---|---|
| DNA Sequence | ORF Start: at 7 | ORF Stop: at 895 |

GGATCCGCGGACACATGTCCAGAGGTGAAGGTGGTGGGCCTGGAGGGCTCTGACAAGCTCACCATTCT
CCGAGGCTGCCCGGGGCTGCCCGGGGCCCCAGGGCCAAAGGGAGAGGCAGGTGTCATTGGAGAGAGAG
GAGAACGCGGTCTCCCTGGAGCCCCTGGAAAGGCAGGACCAGTGGGGCCCAAAGGAGACCGAGGAGAG
AAGGGGATGCGTGGAGAGAAAGGAGACGCTGGGCAGTCTCAGTCGTGTGCGACAGGCCCACGCAACTG
CAAGGACCTGCTAGACCGGGGGTATTTCCTGAGCGGCTGGCACACCATCTACCTGCCCGACTGCCGGC
CCCTGACTGTGCTCTGTGACATGGACACGGACGGAGGGGGCTGGACCGTTTTCCAGCGGAGGATGGAT
GGCTCTGTGGACTTCTATCGGGACTGGGCCGCATACAAGCAGGGCTTCGGCAGTCAGCTGGGGGAGTT
CTGGCTGGGGAACGACAACATCCACGCCCTGACTGCCCAGGGAAGCAGCGAGCTCCGTGTAGACCTGG
TACAAGCTGGTACTGGGAGCCTTTGTCGGGGGCAGTGCGGGTAATTCTCTAACGGGCCACAACAACAA
CCTGGTGGTACGCCGACTGTCATGCTTCAAACCTCAATGGTCTCTACCTCATGGGACCCCATGAGAGC
TATGCCAATGGTATCAACTGGAGTGCGGCGAAGGGGTACAAATATAGCTACAAGGTGTCAGAGATGAA
GGGGCCCGCCCTCGAG

NOV39l, CG56653-05

| | | | |
|---|---|---|---|
| Protein Sequence | SEQ ID NO: 920 | 296 aa | MW at 31819.1kD |

ADTCPEVKVVGLEGSDKLTILRGCPGLPGAPGPKGEAGVIGERGERGLPGAPGKAGPVGPKGDRGEKG
MRGEKGDAGQSQSCATGPRNCKDLLDRGYFLSGWHTIYLPDCRPLTVLCDMDTDGGGWTVFQRRMDGS
VDFYRDWAAYKQGFGSQLGEFWLGNDNIHALTAQGSSELRVDLVDFEGNHQFAKYKSFKVADEAEKYK
LVLGAFVGGSAGNSLTGHNNNFFSTKDQDNDVSSSNCAEKFQGAWWYADCHASNLNGLYLMGPHESYA
NGINWSAAKGYKYSYKVSEMKGPA

| | | |
|---|---|---|
| NOV39m, CG56653-06 | SEQ ID NO: 921 | 988 bp |

| | | |
|---|---|---|
| DNA Sequence | ORF Start: ATG at 56 | ORF Stop: TAG at 905 |

GTTTCCTCTGCCCTGTTAGAGCTGGGGGACTCTTCAGAGTCAAAGGCCAGAGAGCATGGAGCTGAGTG
GAGCCACCATGGCCCGGGGGCTCGCTGTCCTGCTAGTCTTGTTCCTGCATATCAAGAACCTGCCTGCC
CAGGCTGCGGACACATGTCCAGAGGTGAAGGTGGTGGGCCTGGAGGGCTCTGACAAGCTCACCATTCT
CCGAGGCTGCCCGGGGCTGCCCGGGGCCCCAGGGCCAAAGGGAGAGGCAGGTGTCATTGGAGAGAGAG
GAGAACGCGGTCTCCCTGGAGCCCCTGGAAAGGCAGGACCAGTGGGGCCCAAAGGAGACCGAGGAGAG
AAGGGGATGCGTGGAGAGAAAGGAGACGCTGGGCAGTCTCAGTCGTGTGCGACAGGTCCACGCAACTG
CAAGGACCTGCTAGACCGGGGGTATTTCCTGAGCGGCTGGCACACCATCTACCTGCCCGACTGCCGGC
CCCTGACTGTGCTCTGTGACATGGACACGGACGGAGGGGGCTGGACCGTTTTCCAGCGGAGGATGGAT
GGCTCTGTGGACTTTGAGGGCAACCACCAGTTTGCTAAGTACAAATCATTCAAGGTGGCTGACGAGGC
AGAGAAGTACAAGCTGGTACTGGGAGCCTTTGTCGGGGGCAGTGCGGGTAATTCTCTAACGGGCCACA
ACAACAACTTCTTCTCCACCAAAGACCAAGACAATGATGTGAGTTCTTCGAATTGTGCTGAGAAGTTC
CAAGGAGCCTGGTGGTACGCCGACTGTCATGCTTCAAACCTCAATGGTCTCTACCTCATGGGACCCCA
TGAGAGCTATGCCAATGGTATCAACTGCAGTGCGGCGAAGGGGTACAAATATAGCTACAAGGTGTCAG
AGATGAAGGTGCGGCCCGCCTAGACGGGCCAGGACCCCTCCACATGCACCTGCTAGTGGGGAGGCCAC
ACCCACAAGCGCTGCGTCGTGGAAGTCACCCATTTC

NOV39m, CG56653-06

| | | | |
|---|---|---|---|
| Protein Sequence | SEQ ID NO: 922 | 283 aa | MW at 30136.7kD |

MELSGATMARGLAVLLVLFLHIKNLPAQAADTCPEVKVVGLEGSDKLTILRGCPGLPGAPGPKGEAGV
IGERGERGLPGAPGKAGPVGPKGDRGEKGMRGEKGDAGQSQSCATGPRNCKDLLDRGYFLSGWHTIYL
PDCRPLTVLCDMDTDGGGWTVFQRRMDGSVDFEGNHQFAKYKSFKVADEAEKYKLVLGAFVGGSAGNS
LTGHNNNFFSTKDQDNDVSSSNCAEKFQGAWWYADCHASNLNGLYLMGPHESYANGINCSAAKGYKYS
YKVSEMKVRPA

| | | |
|---|---|---|
| NOV39n, CG56653-07 | SEQ ID NO: 923 | 970 bp |

| | | |
|---|---|---|
| DNA Sequence | ORF Start: at 2 | ORF Stop: TAG at 950 |

GGGGCTCGCTGTCATGCTAGTCTTGTTCCTGCATATCAAGAACCTGCCTGCCCAGGCTGCGGACACAT
GTCCAGAGGTGAAGGTGGTGGGCCTGGAGGGCTCTGACAAGCTCACCATTCTCCGAGGCTGCCCGGGG
CTGCCCGGGGCCCCAGGGCCAAAGGGAGAGGCAGGTGTCATTGGAGAGAGAGGAGAACGCGGTCTCCC
TGGAGCCCCTGGAAAGGCAGGACCAGTGGGGCCCAAAGGAGACCGAGGAGAGAAGGGGATGCGTGGAG
AGAAAGGAGACGCTGGGCAGTCTCAGTCGTGTGCGACAGGCCCACGCAACTGCAAGGACCTGCTAGAC
CGGGGGTATTTCCTGAGCGGCTGGCACACCATCTACCTGCCCGACTGCCGGCCCCTGACTGTGCTCTG
TGACATGGACACGGACGGAGGGGGCTGGACCGTTTTCCAGCGGAGGATGGATGGCTCTGTGGACTTCT
ATCGGGACTGGGCCGCATACAAGCAGGGCTTCGGCAGTCAGCTGGGGGAGTTCTGGCTGGGGAATGAC
AACATCCACGCCTTGACTGCCCAGGGAAGCAGCGAGCTCCGTGTAGACCTGGTGGACTTTGAGGGCAA
CCACCAGTTTGCTAAGTACAAATCATTCAAGGTGGCTGACGAGGCAGAGAAGTACAAGCTGGTACTGG
GAGCCTTTGTCGGGGGCAGTGCGGGTAATTCTCTAACGGGCCACAACAACAACTTCTTCTCCACCAAA
GACCAAGACAATGATGTGAGTTCTTCGAATTGTGCTGAGAAGTTCCAAGGAGCCTGGTGGTACGCCGA
CTGTCATGCTTCAAACCTCAATGGTCTCTACCTCATGGGACCCCATGAGAGCTATGCCAATGGTATCA
ACTGGAGTGCGGCGAAGGGGTACAAATATAGCTACAAGGTGTCAGAGATGAAGGTGCGGCCCGCCTAG
ACGGGCCAGGACCCCTCC

NOV39n, CG56653-07

| | | | |
|---|---|---|---|
| Protein Sequence | SEQ ID NO: 924 | 316 aa | MW at 34047.9kD |

GLAVMLVLFLHIKNLPAQAADTCPEVKVVGLEGSDKLTTLRGCPGLPGAPGPKGEAGVIGERGERGLP
DMDTDGGGWTVFQRRMDGSVDFYRDWAAYKQGFGSQLGEFWLGNDNIHALTAQGSSELRVDLVDFEGN
HQFAKYKSFKVADEAEKYKLVLGAFVGGSAGNSLTGHNNNFFSTKDQDNDVSSSNCAEKFQGAWWYAD
CHASNLNGLYLMGPHESYANGINWSAAKGYKYSYKVSEMKVRPA

| | | |
|---|---|---|
| NOV39o, CG56653-09 | SEQ ID NO: 925 | 1144 bp |

| | | |
|---|---|---|
| DNA Sequence | ORF Start: ATG at 183 | ORF Stop: TAG at 981 |

TTTTAGGTCTGTTTGTCGTAGGCAGATGGAGCTTGTTATAATTATGCCTCATAGGGATAGTACAAGGA
GCCCTGTTAGAGCTGGGGGACTCTTCAGAGTCAAAGGCCAGAGAGCATGGAGCTGAGTGGAGCCACCA
TGGCCCGGGGGCTCGCTGTCCTGCTAGTCTTGTTCCTGCATATCAAGAACCTGCCTGCCCAGGCTGCG
GACACATGTCCAGAGGTGAAGGTGGTGGGCCTGGAGGGCTCTGACAAGCTCACCATTCTCCGAGGCTG
CCCGGGGCTGCCCGGGGCCCCAGGGCCAAAGGGAGAGGCAGGTGTCATTGGAGAGAGAGGAGAACGCG
GTCTCCCTGGAGCCCCTGGAAAGGCAGGACCAGTGGGGCCCAAAGGAGACCGAGGAGAGAAGGGGATG
CGTGGAGAGAAAGGAGACGCTGGGCAGTCTCAGTCGTGTGCGACAGGCCCACGCAACTGCAAGGACCT
GCTAGACCGGGGGTATTTCCTGAGCGGCTGGCACACCATCTACCTGCCCGACTGCCGGCCCCTGACTG
TGCTCTGTGACATGGACACGGACGGAGGGGGCTGGACCGTTTTCCAGCGGAGGATGGATGGCTCTGTG
GACTTCTATCGGGACTGGGCCGCATACAAGCAGGGCTTCGGCAGTCAGCTGGGGGGTAATTCTCTAAC
GGGCCACAACAACAACTTCTTCTCCACCAAAGACCAAGACAATGATGTGAGTTCTTCGAATTGTGCTG
AGAAGTTCCAAGGAGCCTGGTGGTACGCCGACTGTCATGCTTCAAACCTCAATGGTCTCTACCTCATG
GGACCCCATGAGAGCTATGCCAATGGTATCAACTGGAGTGCGGCGAAGGGGTACAAATATAGCTACAA
GGTGTCAGAGATGAAGGTGCGGCCCGCCTAQACGGGCCAGGACCCCTCCACATGCACCTGCTAGTGGG
GAGGCCACACCCACAAGCGCTGCGTCGTGGAAGTCACCCCATTTCCCCAGCCAGACACACTCCCATGA
CGCCCACAGCTGCCCCTTTGCCCCCAGCTCAGTCAAGCCGCCACATGCCCACAACC

NOV39o, CG56653-09

| | | | |
|---|---|---|---|
| Protein Sequence | SEQ ID NO: 926 | 266 aa | MW at 28476.8kD |

MELSGATMARGLAVLLVLFLHIKNLPAQAADTCPEVKVVGLEGSDKLTILRGCPGLPGAPGPKGEAGV
IGERGERGLPGAPGKAGPVGPKGDRGEKGMRGEKGDAGQSQSCATGPRNCKDLLDRGYFLSGWHTIYL
PDCRPLTVLCDMDTDGGGWTVFQRRMDGSVDFYRDWAAYKQGFGSQLGGNSLTGHNNNFFSTKDQDND
VSSSNCAEKFQGAWWYADCHASNLNGLYLMGPHESYANGINWSAAKGYKYSYKVSEMKVRPA

| | | |
|---|---|---|
| NOV39p, CG56653-10 | SEQ ID NO: 927 | 651 bp |

| | | |
|---|---|---|
| DNA Sequence | ORF Start: at 7 | ORF Stop: at 646 |

GGATCCGGCCCACGCAACTGCAAGGACCTGCTAGACCGAGGGCACTTCCTGAGCGGCTGGCACACCAT
CTACCTGCCCGACTGCCGGCCCCTGACTGTGCTCTGTGACATGGACACGGACGGAGGGGGCTGGACCG
TTTTCCAGCGGAGGATGGATGGCTCTGTGGACTTCTATCGGGACTGGGCCGCATACAAGCAGGGCTTC
GGCAGTCAGCTGGGGGAGTTCTGGCTGGGGAACGACAACATCCACGCCCTGACTGCCCAGGGAAGCAG
CGAGCTCCGTGTAGACCTGGTGGACTTTGAGGGCAACCACCAGTTTGCTAAGTACAAATCATTCAAGG
TGGCTGACGAGGCAGAGAAGTACAAGCTGGTACTGGGAGCCTTTGTCGGGGGCAGTGCGGGTAATTCT
CTAACGGGCCACAACAACAACTTCTTCTCCACCAAAGACCAAGACAATGATGTGAGTTCTTCGAATTG
TGCTGAGAAGTTCCAAGGAGCCTGGTGGTACGCCGACTGTCATGCTTCAAACCTCAATGGTCTCTACC
TCATGGGACCCCATGAGAGCTATGCCAATGGTATCAACTGGAGTGCGGCGAAGGGGTACAAATATAGC
TACAAGGTGTCAGAGATGAAGGTGCGGCCCGCCCTCGAG

NOV39p, CG56653-10

| | | | |
|---|---|---|---|
| Protein Sequence | SEQ ID NO: 928 | 213 aa | MW at 23830.2kD |

GPRNCKDLLDRGHFLSGWHTIYLPDCRPLTVLCDMDTDGGGWTVFQRRMDGSVDFYRDWAAYKQGFGS
QLGEFWLGNDNIHALTAQGSSELRVDLVDFEGNHQFAKYKSFKVADEAEKYKLVLGAFVGGSAGNSLT
GHNNNFFSTKDQDNDVSSSNCAEKFQGAWWYADCHASNLNGLYLMGPHESYANGINWSAAKGYKYSYK
VSEMKVRPA

| | | |
|---|---|---|
| NOV39q, CG56653-11 | SEQ ID NO: 929 | 1045 bp |

| | | |
|---|---|---|
| DNA Sequence | ORF Start: ATG at 38 | ORF Stop: TAG at 1016 |

GAGCTGGGGGACTCTTCAGAGTCAAAGGCCAGAGAGCATGGAGCTGAGTGGAGCCACCATGGCCCGGG
GGCTCGCTGTCCTGCTAGTCTTGTTCCTGCATATCAAGAACCTGCCTGCCCAGGCTGCGGACACATGT
CCAGAGGTGAAGGTGGTGGGCCTGGAGGGCTCTGACAAGCTCACCATTCTCCGAGGCTGCCCGGGGCT
GCCCGGGGCCCCAGGGCCAAAGGGAGAGGCAGGTGTCATTGGAGAGAGAGGAGAACGCGGTCTCCCTG
GAGCCCCTGGAAAGGCAGGACCAGTGGGGCCCAAAGGAGACCGAGGAGAGAAGGGGATGCGTGGAGAG
AAAGGAGACGCTGGGCAGTCTCAGTCGTGTGCGACAGGCCCACGCAACTGCAAGGACCTGCTAGACCG
GGGGTATTTCCTGAGCGGCTGGCACACCATCTACCTGCCCGACTGCCGGCCCCTGACTGTGCTCTGTG
ACATGGACACGGACGGAGGGGGCTGGACCGTTTTCCAGCGGAGGATGGATGGCTCTGTGGACTTCTAT
CGGGACTGGGCCGCATACAAGCAGGGCTTCGGCAGTCAGCTGGGGGAGTTCTGGCTGGGGAACGACAA
CATCCACGCCCTGACTGCCCAGGGAAGCAGCGAGCTCCGTGTAGACCTGGTGGACTTTGAGGGCAACC
ACCAGTTTGCTAAGTACAAATCATTCAAGGTGGCTGACGAGGCAGAGAAGTACAAGCTGGTACTGGGA
GCCTTTGTCGGGGGCAGTGCGGGTAATTCTCTAACGGGCCACAACAACAACTTCTTCTCCACCAAAGA
CCAAGACAATGATGTGAGTTCTTCGAATTGTGCTGAGAAGTTCCAGGGAGCCTGGTGGTACGCCGACT
GTCATGCTTCAAACCTCAATGGTCTCTACCTCATGGGACCCCATGAGAGCTATGCCAATGGTATCAAC
TGGAGTGCGGCGAAGGGGTACAAATATAGCTACAAGGTGTCAGAGATGAAGGTGCGGCCCGCCTAGAC
GGGCCAGGACCCCTCCACATGCACC

NOV39q, CG56653-11

| | | | |
|---|---|---|---|
| Protein Sequence | SEQ ID NO: 930 | 326 aa | MW at 35078.1kD |

MELSGATMARGLAVLLVLFLHIKNLPAQAADTCPEVKVVGLEGSDKLTILRGCPGLPGAPGPKGEAGV
IGERGERGLPGAPGKAGPVGPKGDRGEKGMRGEKGDAGQSQSCATGPRNCKDLLDRGYFLSGWHTIYL
PDCRPLTVLCDMDTDGGGWTVFQRRMDGSVDFYRDWAAYKQGFGSQLGEFWLGNDNIHALTAQGSSEL
RVDLVDFEGNHQFAKYKSFKVADEAEKYKLVLGAFVGGSAGNSLTGHNNNFFSTKDQDNDVSSSNCAE
KFQGAWWYADCHASNLNGLYLMGPHESYANGINWSAAKGYKYSYKVSEMKVRPA

| | | |
|---|---|---|
| NOV39r, CG56653-12 | SEQ ID NO: 931 | 972 bp |

| | | |
|---|---|---|
| DNA Sequence | ORF Start: ATG at 10 | ORF Stop: at 967 |

GGATCCACCATGGCCCGGGGGCTCGCTGTCCTGCTAGTCTTGTTCCTGCATATCAAGAACCTGCCTGC
CCAGGCTGCGGACACATGTCCAGAGGTGAAGGTGGTGGGCCTGGAGGGCTCTGACAAGCTCACCATTC
TCCGAGGCTGCCCGGGGCTGCCCGGGGCCCCAGGGCCAAAGGGAGAGGCAGGTGTCATTGGAGAGAGA
GGAGAACGCGGTCTCCCTGGAGCCCCTGGAAAGGCAGGACCAGTGGGGCCCAAAGGAGACCGAGGAGA
GAAGGGGATGCGTGGAGAGAAAGGAGACGCTGGGCAGTCTCAGTCGTGTGCGACAGGCCCACGCAACT
GCAAGGACCTGCTAGACCGGGGGTATTTCCTGAGCGGCTGGCACACCATCTACCTGCCCGACTGCCGG
CCCCTGACTGTGCTCTGTGACATGGACACGGACGGAGGGGGCTGGACCGTTTTCCAGCGGAGGATGGA
TGGCTCTGTGGACTTCTATCGGGACTGGGCCGCATACAAGCAGGGCTTCGGCAGTCAGCTGGGGGAGT
TCTGGCTGGGGAACGACAACATCCACGCCCTGACTGCCCAGGGAAGCAGCGAGCTCCGTGTAGACCTG
GTGGACTTTGAGGGCAACCACCAGTTTGCTAAGTACAAATCATTCAAGGTGGCTGACGAGGCAGAGAA
GTACAAGCTGGTACTGGGAGCCTTTGTCGGGGGCAGTGCGGGTAATTCTCTAACGGGCCACAACAACA
ACTTCTTCTCCACCAAAGACCAAGACAATGATGTGAGTTCTTCGAATTGTGCTGAGAAGTTCCAGGGA
GCCTGGTGGTACGCCGACTGTCATGCTTCAAACCTCAATGGTCTCTACCTCATGGGACCCCATGAGAG
CTATGCCAATGGTATCAACTGGAGTGCGGCGAAGGGGTACAAATATAGCTACAAGGTGTCAGAGATGA
AGGTGCGGCCCGCCCTCGAG

NOV39r, CG56653-12

| | | | |
|---|---|---|---|
| Protein Sequence | SEQ ID NO: 932 | 319 aa | MW at 34388.3kD |

MARGLAVLLVLFLHIKNLPAQAADTCPEVKVVGLEGSDKLTILRGCPGLPGAPGPKGEAGVIGERGER
GLPGAPGKAGPVGPKGDRGEKGMRGEKGDAGQSQSCATGPRNCKDLLDRGYFLSGWHTIYLPDCRPLT
VLCDMDTDGGGWTVFQRRMDGSVDFYRDWAAYKQGFGSQLGEFWLGNDNIHALTAQGSSELRVDLVDF
EGNHQFAKYKSFKVADEAEKYKLVLGAFVGGSAGNSLTGHNNNFFSTKDQDNDVSSSNCAEKFQGAWW
YADCHASNLNGLYLMGPHESYANGINWSAAKGYKYSYKVSEMKVRPA

A ClustalW comparison of the above protein sequences yields the following sequence alignment shown in Table 39B.

TABLE 39B
Comparison of the NOV39 protein sequences.

| | |
|---|---|
| NOV39a | MELSGATMARGLAVLLVLFLHIKNLPAQAADTCPEVKVVGLEGSDKLTILRGCPGLPGAP |
| NOV39b | ------------------------------------------------------------ |
| NOV39c | ------------------------------------------------------------ |
| NOV39d | ------------------------------------------------------------ |
| NOV39e | ------------------------------------------------------------ |
| NOV39f | ------------------------------------------------------------ |
| NOV39g | ------------------------------------------------------------ |
| NOV39h | -------MARGLAVLLVLFLHIKNLPAQAADTCPEVKVVGLEGSDKLTILRGCPGLPGAP |
| NOV39i | MELSGATMARGLAVLLVLFLHIKNLPAQAADTCPEVKVVGLEGSGKLTILRGCPGLPGAP |
| NOV39j | -------------------LHIKNLPAQAADTCPEVKVVGLEGSDKLTILRGCPGLPGAP |
| NOV39k | -------MARGLAVLLVLFLHITNLPAQAADTCPEVKVVGLEGSDKLTILRGCPGLPGAP |
| NOV39l | -----------------------------ADTCPEVKVVGLEGSDKLTILRGCPGLPGAP |
| NOV39m | MELSGATMARGLAVLLVLFLHIKNLPAQAADTCPEVKVVGLEGSDKLTILRGCPGLPGAP |
| NOV39n | ----------GLAVMLVLFLHIKNLPAQAADTCPEVKVVGLEGSDKLTTLRGCPGLPGAP |
| NOV39o | MELSGATMARGLAVLLVLFLHIKNLPAQAADTCPEVKVVGLEGSDKLTILRGCPGLPGAP |
| NOV39p | ------------------------------------------------------------ |
| NOV39q | MELSGATMARGLAVLLVLFLHIKNLPAQAADTCPEVKVVGLEGSDKLTILRGCPGLPGAP |
| NOV39r | -------MARGLAVLLVLFLHIKNLPAQAADTCPEVKVVGLEGSDKLTILRGCPGLPGAP |
| NOV39a | GPKGEAGVIGERGERGLPGAPGKAGPVGPKGDRGEKGMRGEKGDAGQSQSCATGPRNCKD |
| NOV39b | ---------------------------------------------------GSGPRNCKD |
| NOV39c | ---------------------------------------------------GSGPRNCKD |
| NOV39d | ---------------------------------------------------GSGPRNCKD |
| NOV39e | ---------------------------------------------------GSGPRNCKD |
| NOV39f | ---------------------------------------------------GSGPRNCKD |
| NOV39g | ---------------------------------------------------GSGPRNCKD |
| NOV39h | GPKGEAGVIGERGERGLPGAPGKAGPVGPKGDRGEKGMRGEKGDAGQSQSCATGPRNCKD |
| NOV39i | GPKGEAGVIGERG------------------DRGEKGMRGEKGDAGQSQSCATGPRNCKD |
| NOV39j | GPKGEAGVIGERGERGLPGAPGKAGPVGPKGDRGEKGMRGEKGDAGQSQSCATGPRNCKD |
| NOV39k | GPKGEAGVIGE------------------------------KGDAGQSQSCATGPRNCKD |
| NOV39l | GPKGEAGVIGERGERGLPGAPGKAGPVGPKGDRGEKGMRGEKGDAGQSQSCATGPRNCKD |
| NOV39m | GPKGEAGVIGERGERGLPGAPGKAGPVGPKGDRGEKGMRGEKGDAGQSQSCATGPRNCKD |
| NOV39n | GPKGEAGVIGERGERGLPGAPGKAGPVGPKGDRGEKGMRGEKGDAGQSQSCATGPRNCKD |
| NOV39o | GPKGEAGVIGERGERGLPGAPGKAGPVGPKGDRGEKGMRGEKGDAGQSQSCATGPRNCKD |
| NOV39p | -----------------------------------------------------GPRNCKD |
| NOV39q | GPKGEAGVIGERGERGLPGAPGKAGPVGPKGDRGEKGMRGEKGDAGQSQSCATGPRNCKD |
| NOV39r | GPKGEAGVIGERGERGLPGAPGKAGPVGPKGDRGEKGMRGEKGDAGQSQSCATGPRNCKD |
| NOV39a | LLDRGYFLSGWHNIYLPDCRPLTVLCDMDTDGGGWTVFQRRMDGSVDFYRDWAAYKQGFG |
| NOV39b | LLDRGYFLSGWHTIYLPDCRPLTVLCDMDTDGGGWTVFQRRMDGSVDFYRDWAAYKQGFG |
| NOV39c | LLDRGYFLSGWHTIYLPDCRPLTVLCDMDTDGGGWTVFQRRMDGSVDFYRDWAAYKQGFG |
| NOV39d | LLDRGYFLSGWHTIYLPDCRPLTVLCDMDTDGGGWTGFQRRMDGSVDFYRDWAAYKQGFG |
| NOV39e | LLDRGYFLSGWHTIYLPDCRPLTVLCDMDTDGGGWTVFQRRMDGSVDFYRDWAAYKQGFG |
| NOV39f | LLDRGYFLSGWHTIYLPDCRPLTVLCDMDTDGGGWTVFQRRMDGSVDFYRDWAAYKQGFG |
| NOV39g | LLDRGHFLSGWHTIYLPDCRPLTVLCDMDTDGGGWTVFQRRMDGSVDFYRDWAAYKQGFG |
| NOV39h | LLDRGYFLSGWHTIYLPDCRPLTVLCDMDTDGGGWTVFQRRMDGSVDFYRDWAAYKQGFG |
| NOV39i | LLDRGYFLSGWHTIYLPDCRPLTVLCDMDTDGGGWTVFQRRMDGSVDFYRDWAAYKQGFG |
| NOV39j | LLDRGYFLSGWHTIYLPDCRPLTVLCDMDTDGGGWTVFQ--------------------- |
| NOV39k | LLDRGYFLSGWHTIYLPDCRPLTVLCDMDTDGGGWTVFQRRMDGSVDFYRDWAAYKQGFG |
| NOV39l | LLDRGYFLSGWHTIYLPDCRPLTVLCDMDTDGGGWTVFQRRMDGSVDFYRDWAAYKQGFG |
| NOV39m | LLDRGYFLSGWHTIYLPDCRPLTVLCDMDTDGGGWTVFQRRMDGSVDFEGNHQFAKYKSF |
| NOV39n | LLDRGYFLSGWHTIYLPDCRPLTVLCDMDTDGGGWTVFQRRMDGSVDFYRDWAAYKQGFG |
| NOV39o | LLDRGYFLSGWHTIYLPDCRPLTVLCDMDTDGGGWTVFQRRMDGSVDFY----------- |
| NOV39p | LLDRGHFLSGWHTIYLPDCRPLTVLCDMDTDGGGWTVFQRRMDGSVDFYRDWAAYKQGFG |
| NOV39q | LLDRGYFLSGWHTIYLPDCRPLTVLCDMDTDGGGWTVFQRRMDGSVDFYRDWAAYKQGFG |
| NOV39r | LLDRGYFLSGWHTIYLPDCRPLTVLCDMDTDGGGWTVFQRRMDGSVDFYRDWAAYKQGFG |
| NOV39a | SQLGEFWLGNDNIHALTAQGSSELRVDLVDFEGNHQFAKYKSFKVADEAEKYKLVLGAFV |
| NOV39b | SQLGEFWLGNDNIHALTAQGSSELRVDLVDFEGNHQFAKYKSFKVADEAEKYKLVLGTFV |
| NOV39c | SQLGEFWLGNDNIHALTAQGSSELRVDLVDFEGNHQFAKYKSFKVADEAEKYKLVLGAFV |
| NOV39d | SQLGEFWLGNDNIHALTAQGSSELRVDLVDFEGNHQFAKYKSFKVADEAEKYKLVLGAFV |
| NOV39e | SRLGEFWLGNDNIHALTAQGSSELRVDLVDFEGNHQFAKYKSFKVADEAEKYKLVLGAFV |
| NOV39f | SQLGEFWLGNDDIHALTAQGSSELRVDLVDFEGNHQFAKYKSFKVADEAEKYKLVLGAFV |
| NOV39g | SQLGEFWLGNDNIHALTAQGSSELRVDLVDFEGNHQFAKYKSFKVADEAEKYKLVLGAFV |
| NOV39h | SQLGEFWLGNDNIHALTAQGSSELRVDLVDFEGNHQFAKYKSFKVADEAEKYKLVLGAFV |
| NOV39i | SQLGEFWLGNDNIHALTAQGSSELRVDLVDFEGNHQFAKYKSFKVADEAEKYKLVLGAFV |
| NOV39j | ------------------------------------------------------------ |
| NOV39k | SQLGEFWLGNDNIHALTAQGSSELRVDLVDFEGNHQFAKYKSFKVADEAEKYKLVLGAFV |
| NOV39l | SQLGEFWLGNDNIHALTAQGSSELRVDLVDFEGNHQFAKYKSFKVADEAEKYKLVLGAFV |
| NOV39m | KVADEAEKYKL------------------------------------V-------LGAFV |
| NOV39n | SQLGEFWLGNDNIHALTAQGSSELRVDLVDFEGNHQFAKYKSFKVADEAEKYKLVLGAFV |
| NOV39o | ------RDWAA------------------------------------Y-------KQGFG |
| NOV39p | SQLGEFWLGNDNIHALTAQGSSELRVDLVDFEGNHQFAKYKSFKVADEAEKYKLVLGAFV |
| NOV39q | SQLGEFWLGNDNIHALTAQGSSELRVDLVDFEGNHQFAKYKSFKVADEAEKYKLVLGAFV |
| NOV39r | SQLGEFWLGNDNIHALTAQGSSELRVDLVDFEGNHQFAKYKSFKVADEAEKYKLVLGAFV |
| NOV39a | GGSAGNSLTGHNNNFFSTKDQDNDVSSSNCAEKFQGAWWYADCHASSLNGLYLMGPHESY |
| NOV39b | GGSAGNSLTGHNNNFFSTKDQDNDVSSSNCAEKFQGAWWYADCHASNLNGLYLMGPHESY |
| NOV39c | GGSAGNSLTGHNNNFFSTKDQDNDVSSSNCAEKFQGAWWYADCHASNLNGLYLMGPHESY |
| NOV39d | GGSAGNSLTGHNNNFFSTKDQDNDVSSSNCAEKFQGAWWYADCHASNLNGLYLMGPHESY |
| NOV39e | GGSAGNSLTGHNNNFFSTKDQDNDVSSSNCAEKFQGAWWYADCHASNLNGLYLMGPHESY |
| NOV39f | GGSAGNSLTGHNNNFFSTKDQDNDVSSSNCAEKFQGAWWYADCHASNLNGLYLMGPHESY |
| NOV39g | GGSAGNSLTGHNNNFFSTKDQDNDVSSSNCAEKFQGAWWYADCHASNLNGLYLMGPHESY |
| NOV39h | GGSAGNSLTGHNNNFFSTKDQDNDVSSSNCAEKFQGAWWYADCHASNLNGLYLMGPHESY |
| NOV39i | GGSAGNSLTGHNNNFFSTKDQDNDVSSSNCAEKFQGAWWYADCHASNLNGLYLMGPHESY |
| NOV39j | ----------------------------------GAWWYADCHASNLNGLYLMGPHESY |
| NOV39k | GGSAGNSLTGHNNNFFSTKDQDNDVSSSNCAEKFQGAWWYADCHASNLNGLYLMGPHESY |
| NOV39l | GGSAGNSLTGHNNNFFSTKDQDNDVSSSNCAEKFQGAWWYADCHASNLNGLYLMGPHESY |
| NOV39m | GGSAGNSLTGHNNNFFSTKDQDNDVSSSNCAEKFQGAWWYADCHASNLNGLYLMGPHESY |
| NOV39n | GGSAGNSLTGHNNNFFSTKDQDNDVSSSNCAEKFQGAWWYADCHASNLNGLYLMGPHESY |
| NOV39o | SQLGGNSLTGHNNNFFSTKDQDNDVSSSNCAEKFQGAWWYADCHASNLNGLYLMGPHESY |
| NOV39p | GGSAGNSLTGHNNNFFSTKDQDNDVSSSNCAEKFQGAWWYADCHASNLNGLYLMGPHESY |
| NOV39q | GGSAGNSLTGHNNNFFSTKDQDNDVSSSNCAEKFQGAWWYADCHASNLNGLYLMGPHESY |
| NOV39r | GGSAGNSLTGHNNNFFSTKDQDNDVSSSNCAEKFQGAWWYADCHASNLNGLYLMGPHESY |
| NOV39a | ANGINWSAAKGYKYSYKVSEMKVRPA-- |
| NOV39b | ANGINWSAAKGYKYSYKVSEMKVRPALE |
| NOV39c | ANGINWSAAKGYKYSYKVSEMKVRPALE |
| NOV39d | ANGINWSAAKGYKYSYKVSEMKVRPALE |
| NOV39e | ANGINWSAAKGYKYSYKVSEMKVRPALE |
| NOV39f | ANGINWSAAKGYKYSYKVSEMKVRPALE |
| NOV39g | ANGINWSAAKGYKYSYKVSEMKVRPALE |
| NOV39h | ANGINWSAAKGYKYSYKVSEMKVRPA-- |
| NOV39i | ANGINWSAAKGYKYSYKVSEMKVRPA-- |
| NOV39j | ANGINWSAAKGYKYSYKVSEMKVRPA-- |
| NOV39k | ANGINWSAAKGYKYSYKVSEMKVRPA-- |
| NOV39l | ANGINWSAAKGYKYSYKVSEMKGPA--- |
| NOV39m | ANGINCSAAKGYKYSYKVSEMKVRPA-- |
| NOV39n | ANGINWSAAKGYKYSYKVSEMKVRPA-- |
| NOV39o | ANGINWSAAKGYKYSYKVSEMKVRPA-- |
| NOV39p | ANGINWSAAKGYKYSYKVSEMKVRPA-- |
| NOV39q | ANGINWSAAKGYKYSYKVSEMKVRPA-- |
| NOV39r | ANGINWSAAKGYKYSYKVSEMKVRPA-- |
| NOV39a | (SEQ ID NO: 898) |
| NOV39b | (SEQ ID NO: 900) |
| NOV39c | (SEQ ID NO: 902) |
| NOV39d | (SEQ ID NO: 904) |
| NOV39e | (SEQ ID NO: 906) |
| NOV39f | (SEQ ID NO: 908) |
| NOV39g | (SEQ ID NO: 910) |
| NOV39h | (SEQ ID NO: 912) |
| NOV39i | (SEQ ID NO: 914) |
| NOV39j | (SEQ ID NO: 916) |
| NOV39k | (SEQ ID NO: 918) |
| NOV39l | (SEQ ID NO: 920) |
| NOV39m | (SEQ ID NO: 922) |
| NOV39n | (SEQ ID NO: 924) |
| NOV39o | (SEQ ID NO: 926) |
| NOV39p | (SEQ ID NO: 928) |
| NOV39q | (SEQ ID NO: 930) |
| NOV39r | (SEQ ID NO: 932) |

Further analysis of the NOV39a protein yielded the following properties shown in Table 39C.

TABLE 39C
Protein Sequence Properties NOV39a

| | |
|---|---|
| SignalP analysis: | Cleavage site between residues 30 and 31 |
| PSORT II analysis: | |

| PSG: | a new signal peptide prediction method | |
|---|---|---|
| | N-region: | length 10; pos. chg 1; neg. chg 1 |
| | H-region: | length 12; peak value 11.35 |
| | PSG score: | 6.95 |

| | |
|---|---|
| GvH: | von Heijne's method for signal seq. recognition |
| | GvH score (threshold: −2.1): −3.79 |
| | possible cleavage site: between 29 and 30 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 1 | | |
| | Number of TMS(s) for threshold 0.5: 1 | | |
| | INTEGRAL | Likelihood = | Transmembrane 6-22 |
| | | −3.35 | |

| | |
|---|---|
| PERIPHERAL | Likelihood = 4.51 (at 234) |

| | |
|---|---|
| | ALOM score: −3.35 (number of TMSs: 1) |
| MTOP: | Prediction of membrane topology (Hartmann et al.) |
| | Center position for calculation: 13 |
| | Charge difference: −1.5 C(−0.5)-N(1.0) |
| | N >= C: N-terminal side will be inside |

>>> membrane topology: type 2 (cytoplasmic tail 1 to 6)

| MITDISC: | discrimination of mitochondrial targeting seq | | | |
|---|---|---|---|---|
| | R content: | 1 | Hyd Moment(75): | 3.15 |
| | Hyd Moment(95): | 5.83 | G content: | 2 |
| | D/E content: | 2 | S/T content: | 2 |

| | |
|---|---|
| | Score: −6.93 |
| Gavel: | prediction of cleavage sites for mitochondrial preseq |
| | R-2 motif at 20 ARG\|LA |

NUCDISC: discrimination of nuclear localization signals

| | |
|---|---|
| | pat4: none |
| | pat7: none |
| | bipartite: none |
| | content of basic residues: 10.7% |
| | NLS Score: −0.47 |
| KDEL: | ER retention motif in the C-terminus: none |

ER Membrane Retention Signals:

KKXX-like motif in the C-terminus: KVRP

SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 89 |
| COIL: | Lupas's algorithm to detect coiled-coil regions |
| | total: 0 residues |

Final Results (k = 9/23):

```
34.8%: mitochondrial
30.4%: cytoplasmic
  8.7%: Golgi
  8.7%: endoplasmic reticulum
  4.3%: vacuolar
  4.3%: extracellular, including cell wall
  4.3%: nuclear
  4.3%: vesicles of secretory system
```

>> prediction for CG56653-08 is mit (k = 23)

A search of the NOV39a protein against the Geneseq database, a proprietary database that contains sequences published in patents and patent publication, yielded several homologous proteins shown in Table 39D.

TABLE 39D
Geneseq Results for NOV39a

| | | NOV39a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAR94183 | Human 35 kDa opsonin protein P35 - | 1 . . . 326 | 248/326 (76%) | e−146 |
| | Homo sapiens, 313 aa. [JP08038182-A, | 1 . . . 313 | 266/326 (81%) | |
| | 13-FEB-1996] | | | |
| AAR30971 | TGF-beta-1 binding protein - Sus | 1 . . . 324 | 239/324 (73%) | e−145 |
| | scrofa, 324 aa. [WO9222319-A1, | 2 . . . 322 | 269/324 (82%) | |
| | 23-DEC-1992] | | | |
| AAR94179 | Human 35 kDa opsonin protein P35 | 28 . . . 326 | 235/299 (78%) | e−140 |
| | fragment (III) residues 26-313 - Homo | 2 . . . 288 | 252/299 (83%) | |
| | sapiens, 288 aa. [JP08038182-A, | | | |
| | 13-FEB-1996] | | | |
| AAR94178 | Human 35 kDa opsonin protein P35 | 109 . . . 326 | 180/218 (82%) | e−110 |
| | fragment (II) residues 96-313 - Homo | 1 . . . 218 | 194/218 (88%) | |
| | sapiens, 218 aa. [JP08038182-A, | | | |
| | 13-FEB-1996] | | | |
| AAB29658 | Human membrane-associated protein | 25 . . . 324 | 143/300 (47%) | 1e−77 |
| | HUMAP-15 - Homo sapiens, 299 aa. | 21 . . . 299 | 189/300 (62%) | |
| | [WO200065054-A2, 02-NOV-2000] | | | |

In a BLAST search of public sequence databases, the NOV39a protein was found to have homology to the proteins shown in the BLASTP data in Table 39E.

TABLE 39E
Public BLASTP Results for NOV39a

| | | NOV39a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| S61517 | ficolin-1 precursor - human, 326 aa. | 1 . . . 326 | 326/326 | (100%) | 0.0 |
| | | 1 . . . 326 | 326/326 | (100%) | |
| O00602 | Ficolin 1 precursor (Collagen/fibrinogen | 1 . . . 326 | 324/326 | (99%) | 0.0 |
| | domain-containing protein 1) (Ficolin-A) | 1 . . . 326 | 325/326 | (99%) | |
| | (Ficolin A) (M-Ficolin) - Homo sapiens | | | | |
| | (Human), 326 aa. | | | | |
| Q29042 | Ficolin - Sus scrofa (Pig), 326 aa. | 1 . . . 324 | 251/324 | (77%) | e−153 |
| | | 1 . . . 324 | 278/324 | (85%) | |
| P57756 | Ficolin 2 precursor (Collagen/fibrinogen | 8 . . . 324 | 242/317 | (76%) | e−150 |
| | domain-containing protein 2) (Ficolin-B) | 1 . . . 317 | 270/317 | (84%) | |
| | (Ficolin B) (Serum lectin p35) (EBP-37) | | | | |
| | (Hucolin) - Rattus norvegicus (Rat), 319 | | | | |
| | aa. | | | | |
| Q29041 | Ficolin - Sus scrofa (Pig), 323 aa. | 1 . . . 324 | 240/324 | (74%) | e−144 |
| | | 1 . . . 321 | 269/324 | (82%) | |

PFam analysis predicts that the NOV39a protein contains the domains shown in the Table 39F.

TABLE 39F
Domain Analysis of NOV39a

| | | Identities/ | | |
|---|---|---|---|---|
| | | Similarities | | |
| | NOV39a Match | for the | | Expect |
| Pfam Domain | Region | Matched Region | | Value |
| Collagen | 50 . . . 108 | 27/60 | (45%) | 3.1e−09 |
| | | 46/60 | (77%) | |
| fibrinogen_C | 114 . . . 326 | 116/272 | (43%) | 2.2e−91 |
| | | 159/272 | (58%) | |

### Example 40

The NOV40 clone was analyzed, and the nucleotide and encoded polypeptide sequences are shown in Table 40A.

TABLE 40A
NOV40 Sequence Analysis

| | | |
|---|---|---|
| NOV40a, CG56806-01 | SEQ ID NO: 933 | 1500 bp |

| | | |
|---|---|---|
| DNA Sequence | ORF Start: ATG at 74 | ORF Stop: TGA at 1490 |

CTTCCGAGCGGGCGCCCGTCCGCCCTGCCGCCGCCGCCGCCGCCGCTTCGCCTGCCGGCCTGAGAGCG
GGACCATGGATGAAAGGTTCAACAAGTGGCTGCTGACGCCGGTGCTCACTCTCCTCTTCGTGGTCATC
ATGTACCAGTACGTGTCCCCCTCCTGCACCAGCTCCTGCACCAACTTCGGGGAGCAGCCCCGCGCGGG
GGAGGCCGGCCCGCCCGCCGTCCCGGGTCCCGCCCGCCGGGCTCAGGCGCCGCCGGAGGAGTGGGAGC
AGAGGAGGCCCCAGTTGCCCCCGCCGCCCCGGGGGCCCCCCGAGGGACCTCGGGGGGCCGCGGCGCCG
GAGGAGGAGGACGAGGAGCCCGGAGACCCCCGGGAGGGGGAGGAAGAGGAGGAGGAAGACGAGCCGGA
CCCCGAGGCCCCGGAAAACGGCTCCCTGCCCCGATTCGTGCCGCGCTTCAACTTCAGCCTGAAGGACC
TGACCCGCTTCGTGGATTTCAACATCAAAGGGCGCGACGTGATCGTGTTCCTCCACATCCAGAAGACG
GGGGGCACCACTTTCGGCCGGCACCTGGTGAAGAACATCCGGCTGGAGCAGCCTTGTAGCTGCAAAGC
CCGGCTGGAGCTGCGGGCTGCACGCCGACTGGACGGAGCTCACCAACTGCGTGCCGGCCATCATGGAG
AAGAAGGACTGTCCCCGCAACCACAGCCACACCAGGAATTTCTATTACATCACAATGTTACGGGATCC
AGTGTCACGTTACCTGAGCGAGTGGAAACATGTCCAGAGAGGGGCCACTTGGAAAACCTCTCTTCATA
TGTGTGATGGAAGAAGCCCCACCCCAGATGAGCTGCCTACCTGCTACCCTGGGGATGACTGGTCTGGG
GTCAGCTTGCGGGAGTTTATGGATTGCACCTACAACCTGGCTAACAATCGCCAGGTGCGCATGCTGGC
TGACCTCAGCCTGGTGGGCTGCTATAACTTGACTTTCATGAACGAGAGTGAAAGAAACACCATCCTGT
TGCAGAGTGCAAAGAACAACCTGAAGAACATGGCCTTCTTTGGGCTCACTGAGTTCCAGAGGAAGACA
CAGTTTCTCTTTGAGAGAACATTCAACCTCAAGTTCATCTCCCCCTTCACACAGTTCAACATCACGCG
GGCTTCTAACGTGGAGATCAACGAGGGTGCCCGCCAACGCATTGAGGATCTAAACTTCCTGGACATGC
AGCTTTACGAGTATGCAAAAGATCTCTTCCAGCAGCGCTACCACCACACCAAGCAGCTAGAGCACCAG
AGGGACCGCCAGAAGCGGCGGGAGGAGCGGAGGCTGCAGCGAGAGCACAGGGACCACCAGTGGCCCAA
AGAAGATGGGGCTGCAGAAGGGACTGTCACCGAGGACTACAACAGCCAGGTGGTGAGATGGTGACCTC
CTGC

NOV40a, CG56806-01

| | | | |
|---|---|---|---|
| Protein Sequence | SEQ ID NO: 934 | 472 aa | MW at 54971.3kD |

MDERFNKWLLTPVLTLLFVVIMYQYVSPSCTSSCTNFGEQPRAGEAGPPAVPGPARRAQAPPEEWEQR
RPQLPPPPRGPPEGPRGAAAPEEEDEEPGDPREGEEEEEEDEPDPEAPENGSLPRFVPRFNFSLKDLT
RFVDFNIKGRDVIVFLHIQKTGGTTFGRHLVKNIRLEQPCSCKAGQKKCTCHRPGKKETWLFSRFSTG
WSCGLHADWTELTNCVPAIMEKKDCPRNHSHTRNFYYITMLRDPVSRYLSEWKHVQRGATWKTSLHMC
DGRSPTPDELPTCYPGDDWSGVSLREFMDCTYNLANNRQVRMLADLSLVGCYNLTFMNESERNTILLQ
SAKNNLKNMAFFGLTEFQRKTQFLFERTFNLKFISPFTQFNITRASNVEINEGARQRIEDLNFLDMQL
YEYAKDLFQQRYHHTKQLEHQRDRQKRREERRLQREHRDHQWPKEDGAAEGTVTEDYNSQVVRW

| | | |
|---|---|---|
| NOV40b, 248061366 | SEQ ID NO: 935 | 750 bp |

| | | |
|---|---|---|
| DNA Sequence | ORF Start: at 1 | ORF Stop: end of sequence |

CGCGGTACCTGTCCCCGCAACCACAGCCACACCAGGAATTTCTATTACATCACAATGTTACGGGATCC
AGTGTCACGTTACCTGAGCGAGTGGAAACATGTCCAGAGAGGGGCCACTTGGAAAACCTCTCTTCATA
TGTGTGATGGAAGAAGCCCCACCCCAGATGGGCTGCCTACCTGCTACCCTGGGGATGACTGGTCTGGG
GTCAGCTTGCGGGAGTTTATGGATTGCACCTACAACCTGGCTAACAATCGCCAGGTGCGCATGCTGGC
TGACCTCAGCCTGGTGGGCTGCTATAACTTGACTTTCATGAACGAGAGTGAAAGAAACACCATCCTGT
TGCAGAGTGCAAAGAACAACCTGAAGAACATGGCCTTCTTTGGGCTCACTGAGTTCCAGAGGAAGACA
CAGTTTCTCTTTGAGAGAACATTCAACCTCAAGTTCATCTCCCCCTTCACACAGTTCAACATCACGCG
GGCTTCTAACGTGGAGATCAACGAGGGTGCCCGCCAACGCATTGAGGATCTAAACTTCCTGGACATGC
AGCTTTACGAGTATGCAAAAGATCTCTTCCAGCAGCGCTACCACCACACCAAGCAGCTAGAGCACCAG
AGGGACCGCCAGAAGCGGCGGGAGGAGCGGAGGCTGCAGCGAGAGCACAGGGACCACCAGTGGCCCAA
AGAAGATGGGGCTGCAGAAGGGACTGTCACCGAGGACTACAACAGCCAGGTGGTGAGATGGCTCGAGG
CG

NOV40b, 248061366

| | | | |
|---|---|---|---|
| Protein Sequence | SEQ ID NO: 936 | 250 aa | MW at 29889.3kD |

RGTCPRNHSHTRNFYYITMLRDPVSRYLSEWKHVQRGATWKTSLHMCDGRSPTPDGLPTCYPGDDWSG
VSLREFMDCTYNLANNRQVRMLADLSLVGCYNLTFMNESERNTILLQSAKNNLKNMAFFGLTEFQRKT
QFLFERTFNLKFISPFTQFNITRASNVEINEGARQRIEDLNFLDMQLYEYAKDLFQQRYHHTKQLEHQ
RDRQKRREERRLQREHRDHQWPKEDGAAEGTVTEDYNSQVVRWLEA

| | | |
|---|---|---|
| NOV40c, 246837961 | SEQ ID NO: 937 | 750 bp |

| | | |
|---|---|---|
| DNA Sequence | ORF Start: at 1 | ORF Stop: end of sequence |

CGCGGTACCTGTCCCCGCAACCACAGCCACACCAGGAATTTCTATTACATCACAATGTTACGGGATCC
AGTGTCACGTTACCTGAGTGAGTGGAAACATGTCCAGAGAGGGGCCACTTGGAAAACCTCTCTTCATA
TGTGTGATGGAAGAAGCCCCACCCCAGATGAGCTGCCTACCTGCTACCCTGGGGATGACTGGTCTGGG
GTCAGCTTGCGGGAGTTTATGGATTGCACCTACAACCTGGCTAACAATCGCCAGGTGCGCATGCTGGC
TGACCTCAGCCTGGTGGGCTGCTATAACTTGACTTTCATGAACGAGAGTGAAAGAAACACCATCCTGT
TGCAGAGTGCAAAGAACAACCTGAAGAACATGGCCTTCTTTGGGCTCACTGAGTTCCAGAGGAAGACA
CAGTTTCTCTTTGAGAGAACATTCAACCTCAAGTTCATCTCCCCCTTCACACAGTTCAACATCACGCG
GGCTTCTAACGTGGAGATCAACGAGGGTGCCCGCCAACGCATTGAGGATCTAAACTTCCTGGACATGC
AGCTTTACGAGTATGCAAAAGATCTCTTCCAGCAGCGCTACCACCACACCAAGCAGCTAGAGCGCCAG
AGGGACCGCCAGAAGCGGCGGGAGGAGCGGAGGCTGCAGCGAGAGCACAGGGACCACCAGTGGCCCAA
AGAAGATGGGGCTGCAGAAGGGACTGTCACCGAGGACTACAACAGCCAGGTGGTGAGATGGCTCGAGG
CG

NOV40c, 246837961

| | | | |
|---|---|---|---|
| Protein Sequence | SEQ ID NO: 938 | 250 aa | MW at 29980.4kD |

RGTCPRNHSHTRNFYYITMLRDPVSRYLSEWKHVQRGATWKTSLHMCDGRSPTPDELPTCYPGDDWSG
VSLREFMDCTYNLANNRQVRLADLSLVGCYNLTFMNESERNTILLQSAKNNLKNMAFFGLTEFQRKT
QFLFERTFNLKFISPFTQFNITRASNVEINEGARQRIEDLNFLDMQLYEYAKDLFQQRYHHTKQLERQ
RDRQKRREERRLQREHRDHQWPKEDGAAEGTVTEDYNSQVVRWLEA

| | | |
|---|---|---|
| NOV40d, 246837965 | SEQ ID NO: 939 | 750 bp |

| | | |
|---|---|---|
| DNA Sequence | ORF Start: at 1 | ORF Stop: end of sequence |

CGCGGTACCTGTCCCCGCAACCACAGCCACACCAGGAATTTCTATTACATCACAATGTTACGGGATCC
AGTGTCACGTTACCTGAGTGAGTGGAAACATGCCCAGAGAGGGGCCACTTGGAAAACCTCTCTTCATA
TGTGTGATGGAAGAAGCCCCACCCCAGATGAGCTGCCTACCTGCTACCCTGGGGATGACTGGTCTGGG
GTCAGCTTGCGGGAGTTTATGGATTGCACCTACAACCTGGCTAACAATCGCCAGGTGCGCATGCTGGC
TGACCTCAGCCTGGTGGGCTGCTATAACTTGACTTTCATGAACGAGAGTGAAAGAAACACCATCCTGT
TGCAGAGTGCAAAGAACAACCTGAAGAACATGGCCTTCTTTGGGCTCACTGAGTTCCAGAGGAAGACA
CAGTTTCTCTTTGAGAGAACATTCAACCTCAAGTTCATCTCCCCCTTCACACAGTTCAACATCACGCG
GGCTTCTAACGTGGAGATCAACGAGGGTGCCCGCCAACGCATTGAGGATCTAAACTTCCTGGACATGC
AGCTTTACGAGTATGCAAAAGATCTCTTCCAGCAGCGCTACCACCACACCAAGCAGCTAGAGCACCAG
AGGGACCGCCAGAAGCGGCGGGAGGAGCGGAGGCTGCAGCGAGAGCACAGGGACCACCAGTGGCCCAA
AGAAGATGGGGCTGCAGAAGGGACTGTCACCGAGGACTACAACAGCCAGGTGGTGAGATGGCTCGAGG
CG

NOV40d, 246837965

| | | | |
|---|---|---|---|
| Protein Sequence | SEQ ID NO: 940 | 250 aa | MW at 29933.3kD |

RGTCPRNHSHTRNFYYITMLRDPVSRYLSEWKHAQRGATWKTSLHMCDGRSPTPDELPTCYPGDDWSG
VSLREFMDCTYNLANNRQVRMLADLSLVGCYNLTFMNESERNTILLQSAKNNLKNMAFFGLTEFQRKT
QFLFERTFNLKFISPFTQFNITRASNVEINEGARQRIEDLNFLDMQLYEYAKDLFQQRYHHTKQLEHQ
RDRQKRREERRLQREHRDHQWPKEDGAAEGTVTEDYNSQVVRWLEA

| | | |
|---|---|---|
| NOV40e, 246837975 | SEQ ID NO: 941 | 750 bp |

| | | |
|---|---|---|
| DNA Sequence | ORF Start: at 1 | ORF Stop: end of sequence |

CGCGGTACCTGTCCCCGCAACCACAGCCACACCAGGAATTTCTATTACATCACAATGTTACGGGATCC
AGTGTCACGTTACCTGAGTGAGTGGAAACATGTCCAGAGAGGGGCCACTTGGAAAACCTCTCTTCATA
TGTGTGATGGAAGAAGCCCCACCCCAGATGAGCTGCCTACCTGCTACCCTGGGGATGACTGGTCTGGG
GTCAGCTTGCGGGAGTTTATGGATTGCACCTACAACCTGGCTAACAATCGCCAGGTGCGCATGCTGGC
TGACCTCAGCCTGGTGGGCTGCTATAACTTGACTTTCATGAACGAGAGTGAAAGAAACACCATCCTGT
TGCAGAGTGCAAAGAACAACCTGAAGAACATGGCCTTCTTTGGGCTCACTGAGTTCCAGAGGAAGACA
CAGTTTCTCTTTGAGAGAGCATTCAACCTCAAGTTCATCTCCCCCTTCACACAGTTCAACATCACGCG
GGCTTCTAACGTGGAGATCAACGAGGGTGCCCACCAACGCATTGAGGATCTAAACTTCCTGGACATGC
AGCTTTACGAGTATGCAAAAGATCTCTTCCAGCAGCGCTACCACCACGCCAAGCAGCTAGAGCACCAG
AGGGACCGCCAGAAGCGGCGGGAGGAGCGGAGGCTGCAGCGAGAGCACAGGGACCACCAGTGGCCCAA
AGAAGATGGGGCTGCAGAAGGGACTGTCACCGAGGACTACAACAGCCAGGTGGTGAGATGGCTCGAGG
CG

NOV40e, 246837975

| | | | |
|---|---|---|---|
| Protein Sequence | SEQ ID NO: 942 | 250 aa | MW at 29882.2kD |

RGTCPRNHSHTPNFYYITMLRDPVSRYLSEWKHVQRGATWKTSLHMCDGRSPTPDELPTCYPGDDWSG
VSLREFMDCTYNLANNRQVRMLADLSLVGCYNLTFMNESERNTILLQSAKNNLKNMAFFGLTEFQRKT
QFLFERAFNLKFISPFTQFNITRASNVEINEGAHQRIEDLNFLDMQLYEYAKDLFQQRYHHAKQLEHQ
RDRQKRREERRLQREHRDHQWPKEDGAAEGTVTEDYNSQVVRWLEA

| | | |
|---|---|---|
| NOV40f, 248061376 | SEQ ID NO: 943 | 750 bp |

| | | |
|---|---|---|
| DNA Sequence | ORF Start: at 1 | ORF Stop: end of sequence |

CGCGGTACCTGTCCCCGCAACCACAGCCACACCAGGAATTTCTATTACATCACAATGTTACGGGATCC
AGTGTCACGTTACCTGAGCGAGTGGAAACATGTCCAGAGAGGGGCCACTTGGAAAACCTCTCTTCATA
TGTGTGATGGAAGAAGCCCCACCCCAGATGAGCTGCCTACCTGCTACCCTGGGGATGACTGGTCTGGG
GTCAGCTTGCGGGAGTTTATGGATTGCACCTACAACCTGGCTAACAATCGCCAGGTGCGCATGCTGGC
TGCAGAGTGCAAAGAACAACCTGAAGAACATGGCCTTCTTTGGGCTCACTGAGTTCCAGAGGAAGACA
CAGTTTCTCTTTGAGAGAACATTCAACCTCAAGTTCATCTCCCCCTTCACACAGTTCAACATCACGCG
GGCTTCTAACGTGGAGATCAACGAGGGTGCCCGCCAACGCATTGAGGATCTAAACTTCCTGGACATGC
AGCTTTACGAGTATGCAAAAGATCTCTTCCAGCAGCGCTACCACCACACCAAGCAGCTAGAGCACCAG
AGGGACCGCCAGAAGCGGCGGGAGGAGCGGAGGCTGCAGCGAGAGCACAGGGACCACCAGTGGCCCAA
AGAAGATGGGGCTGCAGAAGGGACTGTCACCGAGGACTACAACAGCCAGGTGGTGAGATGGCTCGAGG
CG

NOV40f, 248061376

| | | | |
|---|---|---|---|
| Protein Sequence | SEQ ID NO: 944 | 250 aa | MW at 29961.3kD |

RGTCPRNHSHTRNFYYITMLRDPVSRYLSEWKHVQRGATWKTSLHMCDGRSPTPDELPTCYPGDDWSG
VSLREFMDCTYNLANNRQVRMLADLSLVGCYNLTFMNESERNTILLQSAKNNLKNMAFFGLTEFQRKT
QFLFERTFNLKFISPFTQFNITRASNVEINEGARQRIEDLNFLDMQLYEYAKDLFQQRYHHTKQLEHQ
RDRQKRREERRLQREHRDHQWPKEDGAAEGTVTEDYNSQVVRWLEA

| | |
|---|---|
| SEQ ID NO: 945 | 1500 bp |

| | | |
|---|---|---|
| NOV40g, SNP13381685 of | ORF Start: ATG at 74 | ORF Stop: TGA at 1490 |

| | | |
|---|---|---|
| CG56806-01, DNA Sequence | SNP Pos: 870 | SNP Change: A to G |

CTTCCGAGCGGGCGCCCGTCCGCCCTGCCGCCGCCGCCGCCGCCGCTTCGCCTGCCGGCCTGAGAGCG
GGACCATGGATGAAAGGTTCAACAAGTGGCTGCTGACGCCGGTGCTCACTCTCCTCTTCGTGGTCATC
ATGTACCAGTACGTGTCCCCCTCCTGCACCAGCTCCTGCACCAACTTCGGGGAGCAGCCCCGCGCGGG
GGAGGCCGGCCCGCCCGCCGTCCCGGGTCCCGCCCGCCGGGCTCAGGCGCCGCCGGAGGAGTGGGAGC
AGAGGAGGCCCCAGTTGCCCCCGCCGCCCCGGGGGCCCCCCGAGGGACCTCGGGGGGCCGCGGCGCCG
GAGGAGGAGGACGAGGAGCCCGGAGACCCCCGGGAGGGGGAGGAAGAGGAGGAGGAAGACGAGCCGGA
CCCCGAGGCCCCGGAAAACGGCTCCCTGCCCCGATTCGTGCCGCGCTTCAACTTCAGCCTGAAGGACC
TGACCCGCTTCGTGGATTTCAACATCAAAGGGCGCGACGTGATCGTGTTCCTCCACATCCAGAAGACG
GGGGGCACCACTTTCGGCCGGCACCTGGTGAAGAACATCCGGCTGGAGCAGCCTTGTAGCTGCAAAGC
GGGTCAGAAGAAGTGCACCTGCCACCGGCCTGGCAAGAAGGAGACGTGGCTCTTCTCCCGCTTCTCCA
CCGGCTGGAGCTGCGGGCTGCACGCCGACTGGACGGAGCTCACCAACTGCGTGCCGGCCATCATGGAG
AAGAAGGACTGTCCCCGCAACCACAGCCACACCAGGAATTTCTATTACATCACAATGTTACGGGATCC
AGTGTCACGTTACCTGAGCGAGTGGAAACATGTCCAGAGAGGGGCCACTTGGAGAACCTCTCTTCATA
TGTGTGATGGAAGAAGCCCCACCCCAGATGAGCTGCCTACCTGCTACCCTGGGGATGACTGGTCTGGG
GTCAGCTTGCGGGAGTTTATGGATTGCACCTACAACCTGGCTAACAATCGCCAGGTGCGCATGCTGGC
TGACCTCAGCCTGGTGGGCTGCTATAACTTGACTTTCATGAACGAGAGTGAAAGAAACACCATCCTGT
CAGTTTCTCTTTGAGAGAACATTCAACCTCAAGTTCATCTCCCCCTTCACACAGTTCAACATCACGCG
GGCTTCTAACGTGGAGATCAACGAGGGTGCCCGCCAACGCATTGAGGATCTAAACTTCCTGGACATGC
AGCTTTACGAGTATGCAAAAGATCTCTTCCAGCAGCGCTACCACCACACCAAGCAGCTAGAGCACCAG
AGGGACCGCCAGAAGCGGCGGGAGGAGCGGAGGCTGCAGCGAGAGCACAGGGACCACCAGTGGCCCAA
AGAAGATGGGGCTGCAGAAGGGACTGTCACCGAGGACTACAACAGCCAGGTGGTGAGATGGTGACCTC
CTGC

| | | |
|---|---|---|
| NOV40g, SNP13381685 of | SEQ ID NO: 946 | MW at 54999.3kD |

| | | | |
|---|---|---|---|
| CG56806-01, Protein Sequence | SNP Pos: 266 | 472 aa | SNP Change: Lys to Arg |

MDERFNKWLLTPVLTLLFVVIMYQYVSPSCTSSCTNFGEQPRAGEAGPPAVPGPARRAQAPPEEWEQR
RPQLPPPPRGPPEGPRGAAAPEEEDEEPGDPREGEEEEEEDEPDPEAPENGSLPRFVPRFNFSLKDLT
RFVDFNIKGRDVIVFLHIQKTGGTTFGRHLVKNIRLEQPCSCKAGQKKCTCHRPGKKETWLFSRFSTG
WSCGLHADWTELTNCVPAIMEKKDCPRNHSHTRNFYYITMLRDPVSRYLSEWKHVQRGATWRTSLHMC
DGRSPTPDELPTCYPGDDWSGVSLREFMDCTYNLANNRQVRMLADLSLVGCYNLTFMNESERNTILLQ
SAKNNLKNMAFFGLTEFQRKTQFLFERTFNLKFISPFTQFNITRASNVEINEGARQRIEDLNFLDMQL
YEYAKDLFQQRYHHTKQLEHQRDRQKRREERRLQREHRDHQWPKEDGAAEGTVTEDYNSQVVRW

| | |
|---|---|
| SEQ ID NO: 947 | 1500 bp |

| | | |
|---|---|---|
| NOV40h, SNP13381686 of | ORF Start: ATG at 74 | ORF Stop: TGA at 1490 |

| | | |
|---|---|---|
| CG56806-01, DNA Sequence | SNP Pos: 1190 | SNP Change: T to C |

CTTCCGAGCGGGCGCCCGTCCGCCCTGCCGCCGCCGCCGCCGCCGCTTCGCCTGCCGGCCTGAGAGCG
GGACCATGGATGAAAGGTTCAACAAGTGGCTGCTGACGCCGGTGCTCACTCTCCTCTTCGTGGTCATC
ATGTACCAGTACGTGTCCCCCTCCTGCACCAGCTCCTGCACCAACTTCGGGGAGCAGCCCCGCGCGGG
GGAGGCCGGCCCGCCCGCCGTCCCGGGTCCCGCCCGCCGGGCTCAGGCGCCGCCGGAGGAGTGGGAGC
AGAGGAGGCCCCAGTTGCCCCCGCCGCCCCGGGGGCCCCCCGAGGGACCTCGGGGGGCCGCGGCGCCG
GAGGAGGAGGACGAGGAGCCCGGAGACCCCCGGGAGGGGGAGGAAGAGGAGGAGGAAGACGAGCCGGA
CCCCGAGGCCCCGGAAAACGGCTCCCTGCCCCGATTCGTGCCGCGCTTCAACTTCAGCCTGAAGGACC
TGACCCGCTTCGTGGATTTCAACATCAAAGGGCGCGACGTGATCGTGTTCCTCCACATCCAGAAGACG
GGGGGCACCACTTTCGGCCGGCACCTGGTGAAGAACATCCGGCTGGAGCAGCCTTGTAGCTGCAAAGC
GGGTCAGAAGAAGTGCACCTGCCACCGGCCTGGCAAGAAGGAGACGTGGCTCTTCTCCCGCTTCTCCA
CCGGCTGGAGCTGCGGGCTGCACGCCGACTGGACGGAGCTCACCAACTGCGTGCCGGCCATCATGGAG
AAGAAGGACTGTCCCCGCAACCACAGCCACACCAGGAATTTCTATTACATCACAATGTTACGGGATCC
AGTGTCACGTTACCTGAGCGAGTGGAAACATGTCCAGAGAGGGGCCACTTGGAAAACCTCTCTTCATA
TGTGTGATGGAAGAAGCCCCACCCCAGATGAGCTGCCTACCTGCTACCCTGGGGATGACTGGTCTGGG
GTCAGCTTGCGGGAGTTTATGGATTGCACCTACAACCTGGCTAACAATCGCCAGGTGCGCATGCTGGC
TGACCTCAGCCTGGTGGGCTGCTATAACTTGACTTTCATGAACGAGAGTGAAAGAAACACCATCCTGT
TGCAGAGTGCAAAGAACAACCTGAAGAACATGGCCTTCTTTGGGCTCACTGAGTTCCAGAGGAAGACA
CAGTTTCTCTTTGAGAGAACATTCAACCTCAAGCTCATCTCCCCCTTCACACAGTTCAACATCACGCG
GGCTTCTAACGTGGAGATCAACGAGGGTGCCCGCCAACGCATTGAGGATCTAAACTTCCTGGACATGC
AGCTTTACGAGTATGCAAAAGATCTCTTCCAGCAGCGCTACCACCACACCAAGCAGCTAGAGCACCAG
AGGGACCGCCAGAAGCGGCGGGAGGAGCGGAGGCTGCAGCGAGAGCACAGGGACCACCAGTGGCCCAA
AGAAGATGGGGCTGCAGAAGGGACTGTCACCGAGGACTACAACAGCCAGGTGGTGAGATGGTGACCTC
CTGC

| | | |
|---|---|---|
| NOV40h, SNP13381686 of | SEQ ID NO: 948 | MW at 54937.3kD |

| | | | |
|---|---|---|---|
| CG56806-01, Protein Sequence | SNP Pos: 373 | 472 aa | SNP Change: Phe to Leu |

MDERFNKWLLTPVLTLLFVVIMYQYVSPSCTSSCTNFGEQPRAGEAGPPAVPGPARPAQAPPEEWEQR
RPQLPPPPRGPPEGPRGAAAPEEEDEEPGDPREGEEEEEEDEPDPEAPENGSLPRFVPRFNFSLKDLT
RFVDFNIKGRDVIVFLHIQKTGGTTFGRHLVKNIRLEQPCSCKAGQKKCTCHRPGKKETWLFSRFSTG
WSCGLHADWTELTNCVPAIMEKKDCPRNHSHTRNFYYITMLRDPVSRYLSEWKHVQRGATWKTSLHMC
DGRSPTPDELPTCYPGDDWSGVSLREFMDCTYNLANNRQVRMLADLSLVGCYNLTFMNESERNTILLQ
SAKNNLKNMAFFGLTEFQRKTQFLFERTFNLKLISPFTQFNITRASNVEINEGARQRIEDLNFLDMQL
YEYAKDLFQQRYHHTKQLEHQRDRQKRREERRLQREHRDHQWPKEDGAAEGTVTEDYNSQVVRW

A ClustalW comparison of the above protein sequences yields the following sequence alignment shown in Table 40B.

TABLE 40B
Comparison of the NOV40 protein sequences.

| | |
|---|---|
| NOV40a | MDERFNKWLLTPVLTLLFVVIMYQYVSPSCTSSCTNFGEQPRAGEAGPPAVPGPARRAQA |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40a | PPEEWEQRRPQLPPPPRGPPEGPRGAAAPEEEDEEPGDPREGEEEEEEDEPDPEAPENGS |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40a | LPRFVPRFNFSLKDLTRFVDFNIKGRDVIVFLHIQKTGGTTFGRHLVKNIRLEQPCSCKA |
| NOV40b | ------------------------------------------------------------ |
| NOV40c | ------------------------------------------------------------ |
| NOV40d | ------------------------------------------------------------ |
| NOV40e | ------------------------------------------------------------ |
| NOV40f | ------------------------------------------------------------ |
| NOV40a | GQKKCTCHRPGKKETWLFSRFSTGWSCGLHADWTELTNCVPAIMEKKDCPRNHSHTRNFY |
| NOV40b | ---------------------------------------------RGTCPRNHSHTRNFY |
| NOV40c | ---------------------------------------------RGTCPRNHSHTRNFY |
| NOV40d | ---------------------------------------------RGTCPRNHSHTRNFY |
| NOV40e | ---------------------------------------------RGTCPRNHSHTRNFY |
| NOV40f | ---------------------------------------------RGTCPRNHSHTRNFY |
| NOV40a | YITMLRDPVSRYLSEWKHVQRGATWKTSLHMCDGRSPTPDELPTCYPGDDWSGVSLREFM |
| NOV40b | YITMLRDPVSRYLSEWKHVQRGATWKTSLHMCDGRSPTPDGLPTCYPGDDWSGVSLREFM |
| NOV40c | YITMLRDPVSRYLSEWKHVQRGATWKTSLHMCDGRSPTPDELPTCYPGDDWSGVSLREFM |
| NOV40d | YITMLRDPVSRYLSEWKHAQRGATWKTSLHMCDGRSPTPDELPTCYPGDDWSGVSLREFM |
| NOV40e | YITMLRDPVSRYLSEWKHVQRGATWKTSLHMCDGRSPTPDELPTCYPGDDWSGVSLREFM |
| NOV40f | YITMLRDPVSRYLSEWKHVQRGATWKTSLHMCDGRSPTPDELPTCYPGDDWSGVSLREFM |
| NOV40a | DCTYNLANNRQVRMLADLSLVGCYNLTFMNESERNTILLQSAKNNLKNMAFFGLTEFQRK |
| NOV40b | DCTYNLANNRQVPMLADLSLVGCYNLTFMNESEENTILLQSAKNNLKNMAFFGLTEFQRK |
| NOV40c | DCTYNLANNRQVPMLADLSLVGCYNLTFMNESEENTILLQSAKNNLKNMAFFGLTEFQRK |
| NOV40d | DCTYNLANNRQVRMLADLSLVGCYNLTFMNESERNTILLQSAKNNLKNMAFFGLTEFQRK |
| NOV40e | DCTYNLANNRQVRMLADLSLVGCYNLTFMNESEENTILLQSAKNNLKNMAFFGLTEFQRK |
| NOV40f | DCTYNLANNRQVRNLADLSLVGCYNLTFMNESERNTILLQSAKNNLKNNAFFGLTEFQRK |
| NOV40a | TQFLFERTFNLKFISPFTQFNITRASNVEINEGARQRIEDLNFLDMQLYEYAKDLFQQRY |
| NOV40b | TQFLFERTFNLKFISPFTQFNITPASNVEINEGARQRIEDLNFLDMQLYEYAKDLFQQRY |
| NOV40c | TQFLFERTFNLKFISPFTQFNITRASNVEINEGARQRIEDLNFLDMQLYEYAKDLFQQRY |
| NOV40d | TQFLFERTFNLKFISPFTQFNITEASNVEINEGARQRIEDLNFLDMQLYEYAKDLFQQRY |
| NOV40e | TQFLFERAFNLKFISPFTQFNITRASNVEINEGAHQRIEDLNFLDMQLYEYAKDLFQQRY |
| NOV40f | TQFLFERTFNLKFISPFTQFNITFASNVEINEGARQRIEDLNFLDMQLYEYAKDLFQQRY |
| NOV40a | HHTKQLEHQRDRQKRREERRLQREHRDHQWPKEDGAAEGTVTEDYNSQVVRW-- |
| NOV40b | HHTKQLEHQRDRQKEREERRLQREHRDHQWPKEDGAAEGTVTEDYNSQVVRWLEA |
| NOV40c | HHTKQLERQRDRQKRREERRLQREHRDHQWPKEDGAAEGTVTEDYNSQVVRWLEA |
| NOV40d | HHTKQLEHQRDRQKRREERRLQREHRDHQWPKEDGAAEGTVTEDYNSQVVRWLEA |
| NOV40e | HHAKQLEHQRDRQKEREERRLQREHRDHQWPKEDGAAEGTVTEDYNSQVVRWLEA |
| NOV40f | HHTKQLEHQRDRQKRREERRLQREHRDHQWPKEDGAAEGTVTEDYNSQVVRWLEA |
| NOV40a | (SEQ ID NO: 934) |
| NOV40b | (SEQ ID NO: 936) |
| NOV40c | (SEQ ID NO: 938) |
| NOV40d | (SEQ ID NO: 940) |
| NOV40e | (SEQ ID NO: 942) |
| NOV40f | (SEQ ID NO: 944) |

Further analysis of the NOV40a protein yielded the following properties shown in Table 40C.

TABLE 40C
Protein Sequence Properties NOV40a

| | |
|---|---|
| SignalP analysis: | Cleavage site between residues 29 and 30 |
| PSORT II analysis: | |

| PSG: | a new signal peptide prediction method | |
|---|---|---|
| | N-region: | length 7; pos. chg 2; neg. chg 2 |
| | H-region: | length 31; peak value 11.61 |
| | PSG score: | 7.21 |

| | |
|---|---|
| GvH: | von Heijne's method for signal seq. recognition |
| | GvH score (threshold: −2.1): −5.24 |
| | possible cleavage site: between 28 and 29 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 1 | | |
| | Number of TMS(s) for threshold 0.5: 1 | | |
| | INTEGRAL | Likelihood = | Transmembrane 8-24 |
| | | −2.76 | |

| | |
|---|---|
| PERIPHERAL | Likelihood =4.77 (at 312) |

| | |
|---|---|
| | ALOM score: −2.76 (number of TMSs: 1) |
| MTOP: | Prediction of membrane topology (Hartmann et al.) |
| | Center position for calculation: 15 |
| | Charge difference: −2.0 C(−1.0)-N(1.0) |
| | N >= C: N-terminal side will be inside |

>>> membrane topology: type 2 (cytoplasmic tail 1 to 8)

| MITDISC: | discrimination of mitochondrial targeting seq | | | |
|---|---|---|---|---|
| | R content: | 0 | Hyd Moment(75): | 8.01 |
| | Hyd Moment(95): | 7.59 | G content: | 0 |
| | D/E content: | 2 | S/T content: | 0 |

| | |
|---|---|
| | Score: −6.61 |
| Gavel: | prediction of cleavage sites for mitochondrial preseq |
| | cleavage site motif not found |

NUCDISC: discrimination of nuclear localization signals

| | |
|---|---|
| | pat4: none |
| | pat7: none |
| | bipartite: none |
| | content of basic residues: 13.6% |
| | NLS Score: −0.47 |
| KDEL: | ER retention motif in the C-terminus: none |

ER Membrane Retention Signals:

XXRR-like motif in the N-terminus: DERF

none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 76.7 |
| COIL: | Lupas's algorithm to detect coiled-coil regions |
| | total: 0 residues |

Final Results (k = 9/23):

```
39.1%: mitochondrial
34.8%: nuclear
13.0%: cytoplasmic
  4.3%: extracellular, including cell wall
  4.3%: Golgi
  4.3%: peroxisomal
```

>> prediction for CG56806-01 is mit (k = 23)

A search of the NOV40a protein against the Geneseq database, a proprietary database that contains sequences published in patents and patent publication, yielded several homologous proteins shown in Table 40D.

TABLE 40D
Geneseq Results for NOV40a

| | | NOV40a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAO26610 | Human 6-OST-3 protein sequence - | 1 . . . 472 | 471/472 (99%) | 0.0 |
| | Homo sapiens, 471 aa. | 1 . . . 471 | 471/472 (99%) | |
| | [WO200279258-A2, 10-OCT-2002] | | | |
| AAY83902 | Mouse HS6ST2 protein - Mus sp, 470 | 1 . . . 472 | 447/472 (94%) | 0.0 |
| | aa. [JP2000060566-A, 29-FEB-2000] | 1 . . . 470 | 458/472 (96%) | |
| ABP71364 | Mouse H6ST-3 protein - Mus sp, 469 | 1 . . . 471 | 446/471 (94%) | 0.0 |
| | aa. [WO2003004610-A2, 16-JAN-2003] | 1 . . . 469 | 457/471 (96%) | |
| ABP71362 | Human AK polypeptide - Homo | 111 . . . 440 | 242/330 (73%) | e−149 |
| | sapiens, 460 aa. [WO2003004610-A2, | 45 . . . 373 | 285/330 (86%) | |
| | 16-JAN-2003] | | | |
| AAO26608 | Human 6-OST-2A protein sequence - | 111 . . . 440 | 242/330 (73%) | e−149 |
| | Homo sapiens, 465 aa. | 51 . . . 379 | 285/330 (86%) | |
| | [WO200279258-A2, 10-OCT-2002] | | | |

In a BLAST search of public sequence databases, the NOV40a protein was found to have homology to the proteins shown in the BLASTP data in Table 40E.

TABLE 40E
Public BLASTP Results for NOV40a

| | | NOV40a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8IZP7 | Heparan sulfate 6-O- | 1 . . . 472 | 469/472 (99%) | 0.0 |
| | sulfotransferase 3 - Homo sapiens | 1 . . . 471 | 469/472 (99%) | |
| | (Human), 471 aa. | | | |
| Q9QYK4 | Heparan sulfate 6-sulfotransferase | 1 . . . 472 | 447/472 (94%) | 0.0 |
| | 3 - Mus musculus (Mouse), 470 aa. | 1 . . . 470 | 458/472 (96%) | |
| AAH47151 | Hs6st2 protein - Mus musculus | 111 . . . 440 | 244/330 (73%) | e−149 |
| | (Mouse), 535 aa (fragment). | 114 . . . 442 | 285/330 (85%) | |
| Q96SJ4 | Hypothetical protein FLJ14814 - | 111 . . . 440 | 242/330 (73%) | e−148 |
| | Homo sapiens (Human), 459 aa. | 45 . . . 373 | 285/330 (86%) | |
| Q8NBN4 | Hypothetical protein | 111 . . . 440 | 241/330 (73%) | e−148 |
| | NT2RP2000358 - Homo sapiens | 51 . . . 379 | 285/330 (86%) | |
| | (Human), 465 aa. | | | |

PFam analysis predicts that the NOV40a protein contains the domains shown in the Table 40F.

TABLE 40F
Domain Analysis of NOV40a

| | | | |
|---|---|---|---|
| Pfam Domain | NOV40a | Identities/ | Expect |
| | Match Region | Similarities | Value |
| | | for the | |
| | | Matched Region | |

### Example 41

The NOV41 clone was analyzed, and the nucleotide and encoded polypeptide sequences are shown in Table 41A.

TABLE 41A
NOV41 Sequence Analysis

| | | |
|---|---|---|
| NOV41a, CG56878-01 | SEQ ID NO: 949 | 2739 bp |

| | | |
|---|---|---|
| DNA Sequence | ORF Start: ATG at 86 | ORF Stop: TGA at 2090 |

TTGCACTCTCCCACACCCTTTTCTTTTCGTCCGCTCTTCGCTTATTTCTCCCGCCGTCTCCTCTGCAT
AAGAAGGGGAACGAAAGATGGCGGCGGAAACGCTGCTGTCCAGTTTGTTAGGACTGCTGCTTCTGGGA
CTCCTGTTACCCGCAAGTCTGACCGGCGGTGTCGGGAGCCTGAACCTGGAGGAGCTGAGTGAGATGCG
TTATGGGATCGAGATCCTGCCGTTGCCTGTCATGGGAGGGCAGAGCCAATCTTCGGACGTGGTGATTG
TCTCCTCTAAGTACAAACAGCGCTATGAGTGTCGCCTGCCAGCTGGAGCTATTCACTTCCAGCGTGAA
AGGGAGGAGGAAACACCTGCTTACCAAGGGCCTGGGATCCCTGAGTTGTTGAGCCCAATGAGAGATGC
TCCCTGCTTGCTGAAGACAAAGGACTGGTGGACATATGAATTCTGTTATGGACGCCACATCCAGCAAT
TGGGATGATGAAACAGCCAAGGCCTCCAAGCAGCATCGTCTTAAACGCTACCACAGCCAGACCTATGG
GTGCAGGTATCTCTGGGGACTACATCGATCGCGTGGACGAGCCCTTGTCCTGCTCTTATGTGCTGACC
ATTCGCACTCCTCGGCTCTGCCCCCACCCTCTCCTCCGGCCCCCACCCAGTGCTGCACCGCAGGCCAT
CCTCTGTCACCCTTCCCTACAGCCTGAGGAGTACATGGCCTACGTTCAGAGGCAAGCCGTAGACTCAA
AGCAGTATGGAGATAAAATCATAGAGGAGCTGCAAGATCTAGGCCCCCAAGTGTGGAGTGAGACCAAG
TCTGGGGTGGCACCCCAAAAGATGGCAGGTGCGAGCCCGACCAAGGATGACAGTAAGGACTCAGATTT
CTGGAAGATGCTTAATGAGCCAGAGGACCAGGCCCCAGGAGGGGAGGAGGTGCCGGCTGAGGAGCAGG
ACCCAAGCCCTGAGGCAGCAGATTCAGCTTCTGGTGCTCCCAATGATTTTCAGAACAACGTGCAGGTC
AAAGTCATTCGAAGCCCTGCGGATTTGATTCGATTCATAGAGGAGCTGAAAGGTGGAACAAAAAAGGG
GAAGCCAAATATAGGCCAAGAGCAGCCTGTGGATGATGCTGCAGAAGTCCCTCAGAGGGAACCAGAGA
AGGAAAGGGGTGATCCAGAACGGCAGAGAGAGATGGAAGAAGAGGAGGATGAGGATGAGGATGAGGAT
GAAGATGAGGATGAACGGCAGTTACTGGGAGAATTTGAGAAGGAACTGGAAGGGATCCTGCTTCCGTC
AGACCGAGACCGGCTCCGTTCGGAGGTGAAGGCTGGCATGGAGCGGGAACTGGAGAACATCATCCAGG
AGACAGAGAAAGAGCTGGACCCAGATGGGCTGAAGAAGGAGTCAGAGCGGGATCGGGCAATGCTGGCT
CTCACATCCACTCTCAACAAACTCATCAAAAGACTGGAGGAAAAACAGAGTCCAGAGCTGGTGAAGAA
GCACAAGAAAAAGAGGGTTGTCCCCAAAAAGCCTCCCCCATCACCCCAACCTACAGAGGAGGATCCTG
AGCACAGAGTCCGGGTCCGGGTCACCAAGCTCCGTCTCGGAGGCCCTAATCAGGATCTGACTGTCCTC
GAGATGAAACGGGAAAACCCACAGCTGAAACAAATCGAGGGGCTGGTGAAGGAGCTGCTGGAGAGGGA
GGGACTCACAGCTGCAGGGAAAATTGAGATCAAAATTGTCCGCCCATGGGCTGAAGGGACTGAAGAGG
GTGCACGTTGGCTGACTGATGAGGACACGAGAAACCTCAAGGAGATCTTCTTCAATATCTTGGTGCCG
GGAGCTGAAGAGGCCCAGAAGGAACGCCAGCGGCAGAAAGAGCTGGAGAGCAATTACCGCCGGGTGTG
GGGCTCTCCAGGTGGGGAGGGCACAGGGGACCTGGACGAATTTGACTTCTG&GACCAACACTACACTT
GACCCTTCACGGAATCCAGACTCTTCCTGGACTGGCTTGCCTCCTCCCCACCTCCCCACCCTGGAACC
CCTGAGGGCCAAACAGCAGAGTGGAGCTGAGCTGTGGACCTCTCGGGCAACTCTGTGGGTGTGGGGGC
CCTGGGTGAATGCTGCTGCCCCTGCTGGCAGCCACCTTGAGACCTCACCGGGCCTGTGATATTTGCTC
TCCTGAACTCTCACTCAATCCTCTTCCTCTCCTCTGTGGCTTTTCCTGTTATTGTCCCCTAATGATAC
GATATTCCCTGCTGCCTACCTGGAGATTCAGTAGGATCTTTTGAGTGGAGGTGGGTAGAGAGAGCAAC
GAGGGCAGGACACTTAGCAGGCACTGAGCAAGCAGGCCCCCACCTGCCCTTAGTGATGTTTGGAGTCG
TTTTACCCTCTTCTATTGAATTGCCTTGGGATTTCCTTCTCCCTTTCCCTGCCCACCCTGTCCCCTAC
AATTTGTGCTTCTGAGTTGAGGAGCCTTCACCTCTGTTGCTGAGGAAATGGTAGAATGCTGCCTATCA
CCTCCAGCACAATCCCAGCGAAAAAGGTGTGAAGCACCCACCATGTTCTTGAACAATCAGGTTTCTAP
ATAAACAACTGGACCATCA

NOV41a, CG56878-01

| | | | |
|---|---|---|---|
| Protein Sequence | SEQ ID NO: 950 | 668 aa | MW at 75659.9kD |

MAAETLLSSLLGLLLLGLLLPASLTGGVGSLNLEELSEMRYGIEILPLPVMGGQSQSSDVVIVSSKYK
QRYECRLPAGAIHFQREREEETPAYQGPGIPELLSPMRDAPCLLKTKDWWTYEFCYGRHIQQYHMEDS
EIKGEVLYLGYYQSAFDWDDETAKASKQHRLKRYHSQTYGNGSKCDLNGRPREAEVRFLCDEGAGISG
DYIDRVDEPLSCSYVLTIRTPRLCPHPLLRPPPSAAPQAILCHPSLQPEEYMAYVQRQAVDSKQYGDK
IIEELQDLGPQVWSETKSGVAPQKMAGASPTKDDSKDSDFWKMLNEPEDQAPGGEEVPAEEQDPSPEA
ADSASGAPNDFQNNVQVKVIRSPADLIRFIEELKGGTKKGKPNIGQEQPVDDAAEVPQREPEKERGDP
ERQREMEEEEDEDEDEDEDEDERQLLGEFEKELEGILLPSDRDRLRSEVKAGMERELENIIQETEKEL
DPDGLKKESERDRAMLALTSTLNKLIKRLEEKQSPELVKKHKKKRVVPKKPPPSPQPTEEDPEHRVRV
RVTKLRLGGPNQDLTVLEMKRENPQLKQIEGLVKELLEREGLTAAGKIEIKIVRPWAEGTEEGARWLT
DEDTRNLKEIFFNILVPGAEEAQKERQRQKELESNYRRVWGSPGGEGTGDLDEFDF

| | | |
|---|---|---|
| NOV41b, 175070399 | SEQ ID NO: 951 | 1773 bp |

| | | |
|---|---|---|
| DNA Sequence | ORF Start: at 1 | ORF Stop: end of sequence |

GGTACCGGCGGTGTCGGGAGCCTGAACCTGGAGGAGCTGAGTGAGATGCGTTATGGGATCGAGATCCT
GCCGTTGCCTGTCATGGGAGGGCAGAGCCAATCTTCGGACGTGGTGATTGTCTCCTCTAAGTACAAAC
AGCGCTATGAGTGTCGCCTGCCAGCTGGAGCTATTCACTTCCAGCGTGAAAGGGAGGAGGAAACACCT
GCTTACCAAGGGCCTGGGATCCCTGAGTTGTTGAGCCCAATGAGAGATGCTCCCTGCTTGCTGAAGAC
AAAGGACTGGTGGACATATGAATTCTGTTATGGACGCCACATCCAGCAATACCACATGGAAGATTCAG
AGATCAAAGGTGAAGTCCTCTATCTCGGCTACTACCAATCAGCCTTCGACTGGGATGATGAAACAGCC
AAGGCCTCCAAGCAGCATCGTCTTAAACGCTACCACAGCCAGACCTATGGCAATGGGTCCAAGTGCGA
CCTTAATGGGAGGCCCCGGGAGGCCGAGGTTCGGTTCCTCTGTGACGAGGGTGCAGGTATCTCTGGGG
ACTACATCGATCGCGTGGACGAGCCCTTGTCCTGCTCTTATGTGCTGACCATTCGCACTCCTCGGCTC
TGCCCCCACCCTCTCCTCCGGCCCCCACCCAGTGCTGCACCGCAGGCCATCCTCTGTCACCCTTCCCT
ACAGCCTGAGGAGTACATGGCCTACGTTCAGAGGCAAGCCGACTCAAAGCAGTATGGAGATAAAATCA
TAGAGGAGCTGCAAGATCTAGGCCCCCAAGTGTGGAGTGAGACCAAGTCTGGGGTGGCACCCCAAAAG
ATGGCAGGTGCGAGCCCGACCAAGGATGACAGTAAGGACTCAGATTTCTGGAAGATGCTTAATGAGCC
AGAGGACCAGGCCCCAGGAGGGGAGGAGGTGCCGGCTGAGGAGCAGGACCCAAGCCCTGAGGCAGCAG
ATTCAGCTTCTGGTGCTCCCAATGATTTTCAGAACAACGTGCAGGTCAAAGTCATTCGAAGCCCTGCG
GATTTGATTCGATTCATAGAGGAGCTGAAAGGTGGAACAAAAAAGGGGAAGCCAAATATGGACCAAGA
GCAGCCTGTGGATGATGCTGCAGAAGTCCCTCAGAGGGAACCAGAGAAGGAAAGGGGTGATCCAGAAC
GGCAGAGAGAGATGGAAGAAGAGGAGGATGAGGATGAGGATGAGGATGAAGATGAGGATGAACGGCAG
TTACTGGGAGAATTTGAGAAGGAACTGGAAGGGATCCTGCTTCCGTCAGACCGAGACCGGCTCCGTTC
GGAGGTGAAGGCTGGCATGGAGCGGGAACTGGAGAACATCATCCAGGAGACAGAGAAAGAGCTGGACC
CAGATGGGCTGAAGAAGGAGTCAGAGCGGGATCGGGCAATGCTGGCTCTCACATCCACTCTCAACAAA
CCCATCAAAAGACTGGAGGAAAAACAGAGTCCAGAGCTGGTGAAGAAGCACAAGAAAAAGAGGGTTGT
CCCCAAAAAGCCTCCCCCATCACCCCAACCTACAGGGAAAATTGAGATCAAAATTGTCCGCCCATGGG
CTGAAGGGACTGAAGAGGGTGCACGTTGGCTGACTGATGAGGACACGAGAAACCTCAAGGAGATCTTC
TTCAATATCTTGGTGCCGGGAGCTGAAGAGGCCCAGAAGGAACGCCAGCGGCAGAAAGAGCTGGAGAG
CAATTACCGCCGGGTGTGGGGCTCTCCAGGTGGGGAGGGCACAGGGGACCTGGACGAATTTGACTTCG
TCGAC

NOV41b, 175070399

| | | | |
|---|---|---|---|
| Protein Sequence | SEQ ID NO: 952 | 591 aa | MW at 67172.0kD |

GTGGVGSLNLEELSEMRYGIEILPLPVMGGQSQSSDVVIVSSKYKQRYECRLPAGAIHFQREREEETP
AYQGPGIPELLSPMRDAPCLLKTKDWWTYEFCYGRHIQQYHMEDSEIKGEVLYLGYYQSAFDWDDETA
KASKQHRLKRYHSQTYGNGSKCDLNGRPREAEVRFLCDEGAGISGDYIDRVDEPLSCSYVLTIRTPRL
CPHPLLRPPPSAAPQAILCHPSLQPEEYMAYVQRQADSKQYGDKTIEELQDLGPQVWSETKSGVAPQK
MAGASPTKDDSKDSDFWKMLNEPEDQAPGGEEVPAEEQDPSPEAADSASGAPNDFQNNVQVKVIRSPA
DLIRFIEELKGGTKKGKPNMDQEQPVDDAAEVPQREPEKERGDPERQREMEEEEDEDEDEDEDEDERQ
LLGEFEKELEGILLPSDRDRLRSEVKAGMERELENIIQETEKELDPDGLKKESERDRAMLALTSTLLNK
PIKRLEEKQSPELVKKHKKKRVVPKKPPPSPQPTGKIEIKIVRPWAEGTEEGARWLTDEDTRNLKEIF
FNILVPGAEEAQKERQRQKELESNYRRVWGSPGGEGTGDLDEFDFVD

| | | |
|---|---|---|
| NOV41c, 175070432 | SEQ ID NO: 953 | 1788 bp |

| | | |
|---|---|---|
| DNA Sequence | ORF Start: at 1 | ORF Stop: end of sequence |

TCAGAATTCGCCCTTGGTACCGGCGGTGTCGGGAGCCTGAACCTGGAGGAGCTGAGTGAGATGCGTTA
TGGGATCGAGATCCTGCCGTTGCCTGTCATGGGAGGGCAGAGCCAATCTTCGGACGTGGTGATTGTCT
CCTCTAAGTACAAACAGCGCTATGAGTGTCGCCTGCCAGCTGGAGCTATTCACTTCCAGCGTGAAAGG
GAGGAGGAAACACCTGCTTACCAAGGGCCTGGGATCCCTGAGTTGTTGAGCCCAATGAGAGATGCTCC
CTGCTTGCTGAAGACAAAGGACTGGTGGACATATGAATTCTGTTATGGACGCCACATCCAGCAATACC
ACATGGAAGATTCAGAGATCAAAGGTGAAGTCCTCTATCTCGGCTACTACCAATCAGCCTTCGACTGG
GATGATGAAACAGCCAAGGCCTCCAAGCAGCATCGTCTTAAACGCTACCACAGCCAGACCTATGGCAA
TGGGTCCAAGTGCGACCTTAATGGGAGGCCCCGGGAGGCCGAGGTTCGGTTCCTCTGTGACGAGGGTG
CAGGTATCTCTGGGGACTACATCGATCGCGTGGACGAGCCCTTGTCCTGCTCTTATGTGCTGACCATT
CGCACTCCTCGGCTCTGCCCCCACCCTCTCCTCCGGCCCCCACCCAGTGCTGCACCGCAGGCCATCCT
ATGGAGATAAAATCATAGAGGAGCTGCAAGATCTAGGCCCCCAAGTGTGGAGTGAGACCAAGTCTGGG
GTGGCACCCCAAAAGATGGCAGGTGCGAGCCCGACCAAGGATGACAGTAAGGACTCAGATTTCTGGAA
GATGCTTAATGAGCCAGAGGACCAGGCCCCAGGAGGGGAGGAGGTGCCGGCTGAGGAGCAGGACCCAA
GCCCTGAGGCAGCAGATTCAGCTTCTGGTGCTCCCAATGATTTTCAGAACAACGTGCAGGTCAAAGTC
AAATATAGGCCAAGAGCAGCCTGTGGATGATGCTGCAGAAGTCCCTCAGAGGGAACCAGAGAAGGAAA
GGGGTGATCCAGAACGGCAGAGAGAGATGGAAGAAGAGGAGGATGAGGATGAGGATGAGGATGAAGAT
GAGGATGAACGGCAGTTACTGGGAGAATTTGAGAAGGAACTGGAAGGGATCCTGCTTCCGTCAGACCG
AGGCCGGCTCCGTTCGGAGGTGAAGGCTGGCATGGAGCGGGAACTGGAAAACATCATCCAGGAGACAG
AGAAAGAGCTGGACCCAGATGGGCTGAAGAAGGAGTCAGAGCGGGATCGGGCAATGCTGGCTCTCACA
TCCACTCTCAACAAACTCATCAAAAGACTGGAGGAAAAACAGAGTCCAGAGCTGGTGAAGAAGCACAA
GAAAAAGAGGGTTGTCCCCAAAAAGCCTCCCCCATCACCCCAACCTACAGGGAAAATTGAGATCAAAA
TTGTCCGCCCATGGGCTGAAGGGACTGAAGAGGGTGCACGTTGGCTGACTGATGAGGACACGAGAAAC
CTCAAGGAGATCTTCTTCAATATCTTGGTGCCGGGAGCTGAAGAGGCCCAGAAGGAACGCCAGCGGCA
GAAAGAGCTGGAGAGCAATTACCGCCGGGTGTGGGGCTCTCCAGGTGGGGAGGGCACAGGGGACCTGG
ACGAATTTGACTTCGTCGAC

NOV41c, 175070432

| | | | |
|---|---|---|---|
| Protein Sequence | SEQ ID NO: 954 | 596 aa | MW at 67601.5kD |

SEFALGTGGVGSLNLEELSEMRYGIEILPLPVMGGQSQSSDVVIVSSKYKQRYECRLPAGAIHFQRER
EEETPAYQGPGIPELLSPMRDAPCLLKTKDWWTYEFCYGRHIQQYHMEDSEIKGEVLYLGYYQSAFDW
DDETAKASKQHRLKRYHSQTYGNGSKCDLNGRPREAEVRFLCDEGAGISGDYIDRVDEPLSCSYVLTI
RTPRLCPHPLLRPPPSAAPQAILCHPSLQPEEYMAYVQRQADSKQYGDKIIEELQDLGPQVWSETKSG
VAPQKMAGASPTKDDSKDSDFWKMLNEPEDQAPGGEEVPAEEQDPSPEAADSASGAPNDFQNNVQVKV
IRSPADLIRFIEELKGGTKKGKPNIGQEQPVDDAAEVPQREPEKERGDPERQREMEEEEDEDEDEDED
EDERQLLGEFEKELEGILLPSDRGRLRSEVKAGMERELENIIQETEKELDPDGLKKESERDRAMLALT
STLNKLIKRLEEKQSPELVKKHKKKRVVPKKPPPSPQPTGKIEIKIVRPWAEGTEEGARWLTDEDTRN
LKEIFFNILVPGAEEAQKERQRQKELESNYRRVWGSPGGEGTGDLDEFDFVD

| | | |
|---|---|---|
| NOV41d, 175070419 | SEQ ID NO: 955 | 1773 bp |

| | | |
|---|---|---|
| DNA Sequence | ORF Start: at 1 | ORF Stop: end of sequence |

GGTACCGGCGGTGTCGGGAGCCTGAACCTGGAGGAGCTGAGTGAGATGCGTTATGGGATCGAGATCCT
GCCGTTGCCTGTCATGGGAGGGCAGAGCCAATCTTCGGACGTGGTGATTGTCTCCTCTAAGTACAAAC
AGCGCTATGAGTGTCGCCTGCCAGCTGGAGCTATTCACTTCCAGCGTGAAAGGGAGGAGGAAACACCT
GCTTACCAAGGGCCTGGGATCCCTGAGTTGTTGAGCCCAATGAGAGATGCTCCCTGCTTGCTGAAGAC
AAAGGACTGGTGGACATATGAATTCTGTTATGGACGCCACATCCAGCAATACCACATGGAAGATTCAG
AGATCAAAGGTGAAGTCCTCTATCTCGGCTACTACCAATCAGCCTTCGACTGGGATGATGAAACAGCC
AAGGCCTCCAAGCAGCATCGTCTTAAACGCTACCACAGCCAGACCTATGGCAATGGGTCCAAGTGCGA
CCTTAATGGGAGGCCCCGGGAGGCCGAGGTTCGGTTCCTCTGTGACGAGGGTGCAGGTATCTCTGGGG
ACTACATCGATCGCGTGGACGAGCCCTTGTCCTGCTCTTATGTGCTGACCATTCGCACTCCTCGGCTC
TGCCCCCACCCTCTCCTCCGGCCCCCACCCAGTGCTGCACCGCAGGCCATCCTCTGTCACCCTTCCCT
ACAGCCTGAGGAGTACATGGCCTACGTTCAGAGGCAAGCCGACTCAAAGCAGTATGGAGATAAAATCA
TAGAGGAGCTGCAAGATCTAGGCCCCCAAGTGTGGAGTGAGACCAAGTCTGGGGTGGCACCCCAAAAG
ATGGCAGGTGCGAGCCCGACCAAGGATGACAGTAAGGACTCAGATTTCTGGAAGATGCTTAATGAGCC
AGAGGACCAGGCCCCAGGAGGGGAGGAGGTGCCGGCTGAGGAGCAGGACCCAAGCCCTGAGGCAGCAG
ATTCAGCTTCTGGTGCTCCCAATGATTTTCAGAACAACGTGCAGGTCAAAGTCATTCGAAGCCCTGCG
GATTTGATTCGATTCATAGAGGAGCTGAAAGGTGGAATAAAAAAGGGGAAGCCAAATATAGGCCAAGA
GCAGCCTGTGGATGATGCTGCAGAAGTCCCTCAGAGGGAACCAGAGAAGGAAAGGGGTGATCCAGAAC
GGCAGAGAGAGATGGAAGAAGAGGAGGATGAGGATGAGGATGAGGATGAAGATGAGGATGAACGGCAG
TTACTGGGAGAATTTGAGAAGGAACTGGAAGGGATCCTGCTTCCGTCAGACCGAGACCGGCTCCGTTC
GGAGGTGAAGGCTGGCATGGAGCGGGAACTGGAAAACATCATCCAGGAGACAGAGAAAGAGCTGGACC
CAGATGGGCTGAAGAAGGAGTCAGAGCGGGATCGGGCAATGCTGGCTCTCACATCCACTCTCAACAAA
CTCATCAAAAGACTGGAGGAAAAACAGAGTCCAGAGCTGGTGAAGAAGCACAAGAAAAAGAGGGTTGT
CCCCAAAAAGCCTCCCCCATCACCCCAACCTACAGGGAAAATTGAGATCAAAATTGTCCGCCCATGGG
CTGAAGGGACTGAAGAGGGTGCACGTTGGCTGACTGATGAGGACACGAGAAACCTCAAGGAGATCTTC
TTCAATATCTTGGTGCCGGGAGCTGAAGAGGCCCAGAAGGAACGCCAGCGGCAGAAAGAGCTGGAGAG
CAATTACCGCCGGGTGTGGGGCTCTCCAGGTGGGGAGGGCACAGGGGACCTGGACGAATTTGACTTCG
TCGAC

NOV41d, 175070419

| | | | |
|---|---|---|---|
| Protein Sequence | SEQ ID NO: 956 | 591 aa | MW at 67124.0kD |

GTGGVGSLNLEELSEMRYGIEILPLPVMGGQSQSSDVVIVSSKYKQRYECRLPAGAIHFQREREEETP
AYQGPGIPELLSPMRDAPCLLKTKDWWTYEFCYGRHIQQYHMEDSEIKGEVLYLGYYQSAFDWDDETA
KASKQHRLKRYHSQTYGNGSKCDLNGRPREAEVRFLCDEGAGISGDYIDRVDEPLSCSYVLTIRTPRL
CPHPLLRPPPSAAPQAILCHPSLQPEEYMAYVQRQADSKQYGDKIIEELQDLGPQVWSETKSGVAPQK
MAGASPTKDDSKDSDFWKMLNEPEDQAPGGEEVPAEEQDPSPEAADSASGAPNDFQNNVQVKVIRSPA
DLIRFIEELKGGIKKGKPNIGQEQPVDDAAEVPQREPEKERGDPERQREMEEEEDEDEDEDEDEDERQ
LLGEFEKELEGILLPSDPDRLRSEVKAGMERELENIIQETEKELDPDGLKKESERDRAMLALTSTLNK
LIKRLEEKQSPELVKKHKKKRVVPKKPPPSPQPTGKIEIKIVRPWAEGTEEGARWLTDEDTRNLKEIF
FNILVPGAEEAQKERQRQKELESNYRRVWGSPGGEGTGDLDEFDFVD

| | | |
|---|---|---|
| NOV41e, 175070438 | SEQ ID NO: 957 | 1938 bp |

| | | |
|---|---|---|
| DNA Sequence | ORF Start: at 1 | ORF Stop: end of sequence |

GGTACCGGCGGTGTCGGGAGCCTGAACCTGGAGGAGCTGAGTGAGATGCGTTATGGGATCGAGATCCT
GCCGTTGCCTGTCATGGGAGGGCAGAGCCAATCTTCGGACGTGGTGATTGTCTCCTCTAAGTACAAAC
AGCGCTATGAGTGTCGCCTGCCAGCTGGAGCTATTCACTTCCAGCGTGAAAGGGAGGAGGAAACACCT
GCTTACCAAGGGCCTGGGATCCCTGAGTTGTTGAGCCCAATGAGAGATGCTCCCTGCTTGCTGAAGAC
AAAGGACTGGTGGACATATGAATTCTGTTATGGACGCCACATCCAGCAATACCACATGGAAGATTCAG
AGATCAAAGGTGAAGTCCTCTATCTCGGCTACTACCAATCAGCCTTCGACTGGGATGATGAAACAGCC
AAGGCCTCCAAGCAGCATCGTCTTAAACGCTACCACAGCCAGACCTATGGCAATGGGTCCAAGTGCGA
CCTTAATGGGAGGCCCCGGGAGGCCGAGGTTCGGTTCCTCTGTGACGAGGGTGCAGGTATCTCTGGGG
ACTACATCGATCGCGTGGACGAGCCCTTGTCCTGCTCTTATGTGCTGACCATTCGCACTCCTCGGCTC
TGCCCCCACCCTCTCCTCCGGCCCCCACCCAGTGCTGCACCGCAGGCCATCCTCTGTCACCCTTCCCT
ACAGCCTGAGGAGTACATGGCCTACGTTCAGAGGCAAGCCGACTCAAAGCAGTATGGAGATAAAATCA
TAGAGGAGCTGCAAGATCTAGGCCCCCAAGTGTGGAGTGAGACCAAGTCTGGGGTGGCACCCCAAAAG
ATGGCAGGTGCGAGCCCGACCAAGGATGACAGTAAGGACTCAGATTTCTGGAAGATGCTTAATGAGCC
AGAGGACCAGGCCCCAGGAGGGGAGGAGGTGCCGGCTGAGGAGCAGGACCCAAGCCCTGAGGCAGCAG
ATTCAGCTTCTGGTGCTCCCAATGATTTTCAGAACAACGTGCAGGTCAAAGTCATTCGAAGCCCTGCG
GATTTGATTCGATTCATAGAGGAGCTGAAAGGTGGAACAAAAAAGGGGAAGCCAAATATAGGCCAAGA
GCAGCCTGTGGATGATGCTGCAGAAGTCCCTCAGAGGGAACCAGAGAAGGAAAGGGGTGATCCAGAAC
GGCAGAGAGAGATGGAAGAAGAGGAGGATGAGGATGAGGATGAGGATGAAGATGAGGATGAACGGCAG
TTACTGGGAGAATTTGAGAAGGAACTGGAAGGGATCCTGCTTCCGTCAGACCGAGACCGGCTCCGTTC
GGAGGTGAAGGCTGGCATGGAGCGGGAACTGGAGAACATCATCCAGGAGACAGAGAAAGAGCTGGACC
CAGATGGGCTGAAGAAGGAGTCAGAGCGGGATCGGGCAATGCTGGCTCTCACATCCACTCTCAACAAA
CTCATCAAAAGACTGGAGGAAAAACAGAGTCCAGAGCTGGTGAAGAAGCACAAGAAAAAGAGGGTTGT
CCCCAAAAAGCCTCCCCCATCACCCCAACCTACAGAGGAGGATCCTGAGCACAGAGTCCGGGTCCGGG
TCACCAAGCTCCGTCTCGGAGGCCCTAATCAGGATCTGACTGTCCTCGAGATGAAACGGGAAAACCCA
CAGCTGAAACAAATCGAGGGGCTGGTGAAGGAGCTGCTGGAGAGGGAGGGACTCACAGCTGCAGGGAA
AATTGAGATCAAAATTGTCCGCCCATGGGCTGAAGGGACTGAAGAGGGTGCACGTTGGCTGACTGATG
AGGACACGAGAAACCTCAAGGAGATCTTCTTCAATATCTTGGTGCCGGGAGCTGAAGAGGCCCAGAAG
GAACGCCAGCGGCAGAAAGAGCTGGAGAGCAATTACCGCCGGGTGTGGGGCTCTCCAGGTGGGGAGGG
CACAGGGGACCTGGACGAATTTGACTTCGTCGAC

NOV41e, 175070438

| | | | |
|---|---|---|---|
| Protein Sequence | SEQ ID NO: 958 | 646 aa | MW at 73427.1kD |

GTGGVGSLNLEELSEMRYGIEILPLPVMGGQSQSSDVVIVSSKYKQRYECRLPAGAIHFQREREEETP
AYQGPGIPELLSPMRDAPCLLKTKDWWTYEFCYGRHIQQYHMEDSEIKGEVLYLGYYQSAFDWDDETA
KASKQHRLKRYHSQTYGNGSKCDLNGRPREAEVRFLCDEGAGISGDYIDRVDEPLSCSYVLTIRTPRL
CPHPLLRPPPSAAPQAILCHPSLQPEEYMAYVQRQADSKQYGDKIIEELQDLGPQVWSETKSGVAPQK
MAGASPTKDDSKDSDFWKMLNEPEDQAPGGEEVPAEEQDPSPEAADSASGAPNDFQNNVQVKVIRSPA
DLIRFIEELKGGTKKGKPNIGQEQPVDDAAEVPQREPEKERGDPERQREMEEEEDEDEDEDEDEDERQ
LLGEFEKELEGILLPSDRDRLRSEVKAGMERELENIIQETEKELDPDGLKKESERDRAMLALTSTLNK
LIKRLEEKQSPELVKKHKKKRVVPKKPPPSPQPTEEDPEHRVRVRVTKLRLGGPNQDLTVLEMKRENP
QLKQIEGLVKELLEREGLTAAGKIEIKIVRPWAEGTEEGARWLTDEDTRNLKEIFFNILVPGAEEAQK
ERQRQKELESNYRRVWGSPGGEGTGDLDEFDFVD

| | | |
|---|---|---|
| NOV41f, 175070408 | SEQ ID NO: 959 | 1938 bp |

| | | |
|---|---|---|
| DNA Sequence | ORF Start: at 1 | ORF Stop: end of sequence |

GGTACCGGCGGTGTCGGGAGCCTGAACCTGGAGGAGCTGAGTGAGATGCGTTATGGGATCGAGATCCT
AGCGCTATGAGTGTCGCCTGCCAGCTGGAGCTATTCACTTCCAGCGTGAAAGGGAGGAGGAAACACCT
GCTTACCAAGGGCCTGGGATCCCTGAGTTGTTGAGCCCAATGAGAGATGCTCCCTGCTTGCTGAAGAC
AAAGGACTGGTGGACATATGAATTCTGTTATGGACGCCACATCCAGCAATACCACATGGAAGATTCAG
AGATCAAAGGTGAAGTCCTCTATCTCGGCTACTACCAATCAGCCTTCGACTGGGATGATGAAACAGCC
AAGGCCTCCAAGCAGCATCGTCTTAAACGCTACCACAGCCAGACCTATGGCAATGGGTCCAAGTGCGA
CCTTAATGGGAGGCCCCGGGAGGCCGAGGTTCGGTTCCTCTGTGACGAGGGTGCAGGTATCTCTGGGG
ACTACATCGATCGCGTGGACGAGCCCTTGTCCTGCTCTTATGTGCTGACCATTCGCACTCCTCGGCTC
TGCCCCCACCCTCTCCTCCGGCCCCCACCCAGTGCTGCACCGCAGGCCATCCTCTGTCACCCTTCCCT
ACAGCCTGAGGAGTACATGGCCTACGTTCAGAGGCAAGCCGACTCAAAGCAGTATGGAGATAAAATCA
TAGAGGAGCTGCAAGATCTAGGCCCCCAAGTGTGGAGTGAGACCAAGTCTGGGGTGGCACCCCAAAAG
ATGGCAGGTGCGAGCCCGACCAAGGATGACAGTAAGGACTCAGATTTCTGGAAGATGCTTAATGAGCC
AGAGGACCAGGCCCCAGGAGGGGAGGAGGTGCCGGCTGAGGAGCAGGACCCAAGCCCTGAGGCAGCAG
ATTCAGCTTCTGGTGCTCCCAATGATTTTCAGAACAACGTGCAGGTCAAAGTCATTCGAAGCCCTGCG
GATTTGATTCGATTCATAGAGGAGCTGAAAGGTGGAACAAAAAAGGGGAAGCCAAATATAGGCCAAGA
GCAGCCTGTGGATGATGCTGCAGAAGTCCCTCAGAGGGAACCAGAGAAGGAAAGGGGTGATCCAGAAC
GGCAGAGAGAGATGGAAGAAGAGGAGGATGAGGATGAGGATGAGGATGAAGATGAGGATGAACGGCAG
TTACTGGGAGAATTTGAGAAGGAACTGGAAGGGATCCTGCTTCCGTCAGACCGAGACCGGCTCCGTTC
GGAGGTGAAGGCTGGCATGGAGCGGGAACTGGAAAACATCATCCAGGAGACAGAGAAAGAGCTGGACC
CAGATGGGCTGAAGAAGGAGTCAGAGCGGGATCGGGCAATGCTGGCTCTCACATCCACTCTCAACAAA
CTCATCAAAAGACTGGAGGAAAAACAGAGTCCAGAGCTGGTGAAGAAGCACAAGAAAAAGAGGGTTGT
CCCCAAAAAGCCTCCCCCATCACCCCAACCTACAGAGGAGGATCCTGAGCACAGAGTCCGGGTCCGGG
TCACCAAGCTCCGTCTCGGAGGCCCTAATCAGGATCTGACTGTCCTCGAGATGAAACGGGAAAACCCA
CAGCTGAAACAAATCGAGGGGCTGGTGAAGGAGCTGCTGGAGAGGGAGGGACTCACAGCTGCAGGGAA
AATTGAGATCAAAATTGTCCGCCCATGGGCTGAAGGGACTGAAGAGGGTGCACGTTGGCTGACTGATG
AGGACACGAGAAACCTCAAGGAGATCTTCTTCAATATCTTGGTGCCGGGAGCTGAAGAGGCCCAGAAG
GAACGCCAGCGGCAGAAAGAGCTGGAGAGCAATTACCGCCGGGTGTGGGGCTCTCCAGGTGGGGAGGG
CACAGGGGACCTGGACGAATTTGACTTCGTCGAC

NOV41f, 175070408

| | | | |
|---|---|---|---|
| Protein Sequence | SEQ ID NO: 960 | 646 aa | MW at 73427.1kD |

GTGGVGSLNLEELSEMRYGIEILPLPVMGGQSQSSDVVIVSSKYKQRYECRLPAGAIHFQREREEETP
AYQGPGIPELLSPMRDAPCLLKTKDWWTYEFCYGRHIQQYHMEDSEIKGEVLYLGYYQSAFDWDDETA
KASKQHRLKRYHSQTYGNGSKCDLNGRPREAEVRFLCDEGAGISGDYIDRVDEPLSCSYVLTIRTPRL
CPHPLLRPPPSAAPQAILCHPSLQPEEYMAYVQRQADSKQYGDKIIEELQDLGPQVWSETKSGVAPQK
MAGASPTKDDSKDSDFWKMLNEPEDQAPGGEEVPAEEQDPSPEAADSASGAPNDFQNNVQVKVIRSPA
DLIRFIEELKGGTKKGKPNIGQEQPVDDAAEVPQREPEKERGDPERQREMEEEEDEDEDEDEDEDERQ
LLGEFEKELEGILLPSDRDRLRSEVKAGMERELENIIQETEKELDPDGLKKESERDRAMLALTSTLNK
LIKRLEEKQSPELVKKHKKKRVTPKKPPPSPQPTEEDPEHRVRVRVTKLRLGGPNQDLTVLEMKRENP
QLKQIEGLVKELLEREGLTAAGKIEIKIVRPWAEGTEEGARWLTDEDTRNLKEIFFNILVPGAEEAQK
ERQRQKELESNYRRVWGSPGGEGTGDLDEFDFVD

| | | |
|---|---|---|
| NOV41g, CG56878-02 | SEQ ID NO: 961 | 1938 bp |

| | | |
|---|---|---|
| DNA Sequence | ORF Start: at 7 | ORF Stop: at 1933 |

GGTACCGGCGGTGTCGGGAGCCTGAACCTGGAGGAGCTGAGTGAGATGCGTTATGGGATCGAGATCCT
GCCGTTGCCTGTCATGGGAGGGCAGAGCCAATCTTCGGACGTGGTGATTGTCTCCTCTAAGTACAAAC
AGCGCTATGAGTGTCGCCTGCCAGCTGGAGCTATTCACTTCCAGCGTGAAAGGGAGGAGGAAACACCT
GCTTACCAAGGGCCTGGGATCCCTGAGTTGTTGAGCCCAATGAGAGATGCTCCCTGCTTGCTGAAGAC
AAAGGACTGGTGGACATATGAATTCTGTTATGGACGCCACATCCAGCAATACCACATGGAAGATTCAG
AGATCAAAGGTGAAGTCCTCTATCTCGGCTACTACCAATCAGCCTTCGACTGGGATGATGAAACAGCC
AAGGCCTCCAAGCAGCATCGTCTTAAACGCTACCACAGCCAGACCTATGGCAATGGGTCCAAGTGCGA
CCTTAATGGGAGGCCCCGGGAGGCCGAGGTTCGGTTCCTCTGTGACGAGGGTGCAGGTATCTCTGGGG
ACTACATCGATCGCGTGGACGAGCCCTTGTCCTGCTCTTATGTGCTGACCATTCGCACTCCTCGGCTC
TGCCCCCACCCTCTCCTCCGGCCCCCACCCAGTGCTGCACCGCAGGCCATCCTCTGTCACCCTTCCCT
ACAGCCTGAGGAGTACATGGCCTACGTTCAGAGGCAAGCCGACTCAAAGCAGTATGGAGATAAAATCA
TAGAGGAGCTGCAAGATCTAGGCCCCCAAGTGTGGAGTGAGACCAAGTCTGGGGTGGCACCCCAAAAG
ATGGCAGGTGCGAGCCCGACCAAGGATGACAGTAAGGACTCAGATTTCTGGAAGATGCTTAATGAGCC
AGAGGACCAGGCCCCAGGAGGGGAGGAGGTGCCGGCTGAGGAGCAGGACCCAAGCCCTGAGGCAGCAG
ATTCAGCTTCTGGTGCTCCCAATGATTTTCAGAACAACGTGCAGGTCAAAGTCATTCGAAGCCCTGCG
GATTTGATTCGATTCATAGAGGAGCTGAAAGGTGGAACAAAAAAGGGGAAGCCAAATATAGGCCAAGA
GCAGCCTGTGGATGATGCTGCAGAAGTCCCTCAGAGGGAACCAGAGAAGGAAAGGGGTGATCCAGAAC
GGCAGAGAGAGATGGAAGAAGAGGAGGATGAGGATGAGGATGAGGATGAAGATGAGGATGAACGGCAG
TTACTGGGAGAATTTGAGAAGGAACTGGAAGGGATCCTGCTTCCGTCAGACCGAGACCGGCTCCGTTC
GGAGGTGAAGGCTGGCATGGAGCGGGAACTGGAGAACATCATCCAGGAGACAGAGAAAGAGCTGGACC
CAGATGGGCTGAAGAAGGAGTCAGAGCGGGATCGGGCAATGCTGGCTCTCACATCCACTCTCAACAAA
CTCATCAAAAGACTGGAGGAAAAACAGAGTCCAGAGCTGGTGAAGAAGCACAAGAAAAAGAGGGTTGT
CCCCAAAAAGCCTCCCCCATCACCCCAACCTACAGAGGAGGATCCTGAGCACAGAGTCCGGGTCCGGG
TCACCAAGCTCCGTCTCGGAGGCCCTAATCAGGATCTGACTGTCCTCGAGATGAAACGGGAAAACCCA
AATTGAGATCAAAATTGTCCGCCCATGGGCTGAAGGGACTGAAGAGGGTGCACGTTGGCTGACTGATG
AGGACACGAGAAACCTCAAGGAGATCTTCTTCAATATCTTGGTGCCGGGAGCTGAAGAGGCCCAGAAG
GAACGCCAGCGGCAGAAAGAGCTGGAGAGCAATTACCGCCGGGTGTGGGGCTCTCCAGGTGGGGAGGG
CACAGGGGACCTGGACGAATTTGACTTCGTCGAC

NOV41g, CG56878-02

| | | | |
|---|---|---|---|
| Protein Sequence | SEQ ID NO: 962 | 642 aa | MW at 73054.8kD |

GGVGSLNLEELSEMRYGIEILPLPVMGGQSQSSDVVIVSSKYKQRYECRLPAGAIHFQREREEETPAY
QGPGIPELLSPMRDAPCLLKTKDWWTYEFCYGRHIQQYHMEDSEIKGEVLYLGYYQSAFDWDDETAKA
SKQHRLKRYHSQTYGNGSKCDLNGRPREAEVRFLCDEGAGISGDYIDRVDEPLSCSYVLTIRTPRLCP
HPLLRPPPSAAPQAILCHPSLQPEEYMAYVQRQADSKQYGDKIIEELQDLGPQVWSETKSGVAPQKMA
GASPTKDDSKDSDFWKMLNEPEDQAPGGEEVPAEEQDPSPEAADSASGAPNDFQNINQVKVIRSPADL
IRFIEELKGGTKKGKPNIGQEQPVDDAAEVPQREPEKERGDPERQREMEEEEDEDEDEDEDEDERQLL
GEFEKELEGILLPSDRDRLRSEVKAGMERELENIIQETEKELDPDGLKKESERDRAMLALTSTLNKLI
KRLEEKQSPELVKKHKKKRVVPKKPPPSPQPTEEDPEHRVRVRVTKLRLGGPNQDLTVLEMKRENPQL
KQIEGLVKELLEREGLTAAGKIEIKIVRPWAEGTEEGARWLTDEDTRNLKEIFFNILVPGAEEAQKER
QRQKELESNYRRVWGSPGGEGTGDLDEFDF

| | | |
|---|---|---|
| NOV41h, CG56878-03 | SEQ ID NO: 963 | 1773 bp |

| | | |
|---|---|---|
| DNA Sequence | ORF Start: at 7 | ORF Stop: at 1768 |

GGTACCGGCGGTGTCGGGAGCCTGAACCTGGAGGAGCTGAGTGAGATGCGTTATGGGATCGAGATCCT
GCCGTTGCCTGTCATGGGAGGGCAGAGCCAATCTTCGGACGTGGTGATTGTCTCCTCTAAGTACAAAC
AGCGCTATGAGTGTCGCCTGCCAGCTGGAGCTATTCACTTCCAGCGTGAAAGGGAGGAGGAAACACCT
GCTTACCAAGGGCCTGGGATCCCTGAGTTGTTGAGCCCAATGAGAGATGCTCCCTGCTTGCTGAAGAC
AAAGGACTGGTGGACATATGAATTCTGTTATGGACGCCACATCCAGCAATACCACATGGAAGATTCAG
AGATCAAAGGTGAAGTCCTCTATCTCGGCTACTACCAATCAGCCTTCGACTGGGATGATGAAACAGCC
AAGGCCTCCAAGCAGCATCGTCTTAAACGCTACCACAGCCAGACCTATGGCAATGGGTCCAAGTGCGA
CCTTAATGGGAGGCCCCGGGAGGCCGAGGTTCGGTTCCTCTGTGACGAGGGTGCAGGTATCTCTGGGG
ACTACATCGATCGCGTGGACGAGCCCTTGTCCTGCTCTTATGTGCTGACCATTCGCACTCCTCGGCTC
TGCCCCCACCCTCTCCTCCGGCCCCCACCCAGTGCTGCACCGCAGGCCATCCTCTGTCACCCTTCCCT
ACAGCCTGAGGAGTACATGGCCTACGTTCAGAGGCAAGCCGACTCAAAGCAGTATGGAGATAAAATCA
TAGAGGAGCTGCAAGATCTAGGCCCCCAAGTGTGGAGTGAGACCAAGTCTGGGGTGGCACCCCAAAAG
ATGGCAGGTGCGAGCCCGACCAAGGATGACAGTAAGGACTCAGATTTCTGGAAGATGCTTAATGAGCC
AGAGGACCAGGCCCCAGGAGGGGAGGAGGTGCCGGCTGAGGAGCAGGACCCAAGCCCTGAGGCAGCAG
ATTCAGCTTCTGGTGCTCCCAATGATTTTCAGAACAACGTGCAGGTCAAAGTCATTCGAAGCCCTGCG
GATTTGATTCGATTCATAGAGGAGCTGAAAGGTGGAATAAAAAAGGGGAAGCCAAATATAGGCCAAGA
GCAGCCTGTGGATGATGCTGCAGAAGTCCCTCAGAGGGAACCAGAGAAGGAAAGGGGTGATCCAGAAC
GGCAGAGAGAGATGGAAGAAGAGGAGGATGAGGATGAGGATGAGGATGAAGATGAGGATGAACGGCAG
TTACTGGGAGAATTTGAGAAGGAACTGGAAGGGATCCTGCTTCCGTCAGACCGAGACCGGCTCCGTTC
GGAGGTGAAGGCTGGCATGGAGCGGGAACTGGAAAACATCATCCAGGAGACAGAGAAAGAGCTGGACC
CAGATGGGCTGAAGAAGGAGTCAGAGCGGGATCGGGCAATGCTGGCTCTCACATCCACTCTCAACAAA
CTCATCAAAAGACTGGAGGAAAAACAGAGTCCAGAGCTGGTGAAGAAGCACAAGAAAAAGAGGGTTGT
CCCCAAAAAGCCTCCCCCATCACCCCAACCTACAGGGAAAATTGAGATCAAAATTGTCCGCCCATGGG
CTGAAGGGACTGAAGAGGGTGCACGTTGGCTGACTGATGAGGACACGAGAAACCTCAAGGAGATCTTC
TTCAATATCTTGGTGCCGGGAGCTGAAGAGGCCCAGAAGGAACGCCAGCGGCAGAAAGAGCTGGAGAG
CAATTACCGCCGGGTGTGGGGCTCTCCAGGTGGGGAGGGCACAGGGGACCTGGACGAATTTGACTTCG
TCGAC

NOV41h, CG56878-03

| | | | |
|---|---|---|---|
| Protein Sequence | SEQ ID NO: 964 | 587 aa | MW at 66751.6kD |

GGVGSLNLEELSEMRYGIETLPLPVMGGQSQSSDVVIVSSKYKQRYECRLPAGAIHFQREREEETPAY
QGPGIPELLSPMRDAPCLLKTKDWWTYEFCYGRHIQQYHMEDSEIKGEVLYLGYYQSAFDWDDETAKA
SKQHRLKRYHSQTYGNGSKCDLNGRPREAEVRFLCDEGAGISGDYIDRVDEPLSCSYVLTIRTPRLCP
HPLLRPPPSAAPQAILCHPSLQPEEYMAYVQRQADSKQYGDKIIEELQDLGPQVWSETKSGVAPQKMA
GASPTKDDSKDSDFWKMLNEPEDQAPGGEEVPAEEQDPSPEAADSASGAPNDFQNNVQVKVIRSPADL
IRFIEELKGGIKKGKPNIGQEQPVDDAAEVPQREPEKERGDPERQREMEEEEDEDEDEDEDEDERQLL
GEFEKELEGILLPSDRDRLRSEVKAGMERELENIIQETEKELDPDGLKKESERDRAMLALTSTLNKLI
KRLEEKQSPELVKKHKKKRVVPKKPPPSPQPTGKIEIKIVRPWAEGTEEGARWLTDEDTHNLKEIFFN
ILVPGAEEAQKERQRQKELESNYRRVWGSPGGEGTGDLDEFDF

| | | |
|---|---|---|
| NOV41i, CG56878-04 | SEQ ID NO: 965 | 2702 bp |

| | | |
|---|---|---|
| DNA Sequence | ORF Start: ATG at 86 | ORF Stop: TGA at 2036 |

TTGCACTCTCCCACACCCTTTTCTTTTCGTCCGCTCTTCGCTTATTTCTCCCGCCGTCTCCTCTGCAT
AAGAAGGGGAACGAAAGATGGCGGCGGAAACGCTGCTGTCCAGTTTGTTAGGACTGCTGCTTCTGGGA
CTCCTGTTACCCGCAAGTCTGACCGGCGGTGTCGGGAGCCTGAACCTGGAGGAGCTGAGTGAGATGCG
TTATGGGATCGAGATCCTGCCGTTGCCTGTCATGGGAGGGCAGAGCCAATCTTCGGACGTGGTGATTG
TCTCCTCTAAGTACAAACAGCGCTATGAGTGTCGCCTGCCAGCTGGAGCTATTCACTTCCAGCGTGAA
AGGGAGGAGGAAACACCTGCTTACCAAGGGCCTGGGATCCCTGAGTTGTTGAGCCCAATGAGAGATGC
TCCCTGCTTGCTGAAGACAAAGGACTGGTGGACATATGAATTCTGTTATGGACGCCACATCCAGCAAT
ACCACATGGAAGATTCAGAGATCAAAGGTGAAGTCCTCTATCTCGGCTACTACCAATCAGCCTTCGAC
TGGGATGATGAAACAGCCAAGGCCTCCAAGCAGCATCGTCTTAAACGCTACCACAGCCAGACCTATGG
CAATGGGTCCAAGTGCGACCTTAATGGGAGGCCCCGGGAGGCCGAGGTTCGGTTCCTCTGTGACGAGG
GTGCAGGTATCTCTGGGGACTACATCGATCGCGTGGACGAGCCCTTGTCCTGCTCTTATGTGCTGACC
CCTCTGTCACCCTTCCCTACAGCCTGAGGAGTACATGGCCTACGTTCAGAGGCAAGCCGACTCAAAGC
AGTATGGAGATAAAATCATAGAGGAGCTGCAAGATCTAGGCCCCCAAGTGTGGAGTGAGACCAAGTCT
GGGGTGGCACCCCAAAAGATGGCAGGTGCGAGCCCGACCAAGGATGACAGTAAGGACTCAGATTTCTG
GAAGATGCTTAATGAGCCAGAGGACCAGGCCCCAGGAGGGGAGGAGGTGCCGGCTGAGGAGCAGGACC
GTCATTCGAAGCCCTGCGGATTTGATTCGATTCATAGAGGAGCTGAAAGGTGGAACAAAAAAGGGGAA
GCCAAATATAGGCCAAGAGCAGCCTGTGGATGATGCTGCAGAAGTCCCTCAGAGGGAACCAGAGAAGG
AAAGGGGTGATCCAGAACGGCAGAGAGAGATGGAAGAAGAGGAGGATGAGGATGAGGATAAGATGAGG
ATGAACGGCAGTTACTGGGGAGAATTTGAGAAGGAACTGGAAGGGATCCTGCTTCCGTCAGACCGAGA
CCGGCTCCGTTCGGAGACAGAGAAAGAGCTGGACCCAGATGGGCTGAAGAAGGAGTCAGAGCGGGATC
GGGCAATGCTGGCTCTCACATCCACTCTCAACAAACTCATCAAAAGACTGGAGGAAAAACAGAGTCCA
GAGCTGGTGAAGAAGCACAAGAAAAAGAGGGTTGTCCCCAAAAAGCCTCCCCCATCACCCCAACCTAC
AGAGGAGGATCCTGAGCACAGAGTCCGGGTCCGGGTCACCAAGCTCCGTCTCGGAGGCCCTAATCAGG
ATCTGACTGTCCTCGAGATGAAACGGGAAAACCCACAGCTGAAACAAATCGAGGGGCTGGTGAAGGAG
CTGCTGGAGAGGGAGGGACTCACAGCTGCAGGGAAAATTGAGATCAAAATTGTCCGCCCATGGGCTGA
AGGGACTGAAGAGGGTGCACGTTGGCTGACTGATGAGGACACGAGAAACCTCAAGGAGATCTTCTTCA
ATATCTTGGTGCCGGGAGCTGAAGAGGCCCAGAAGGAACGCCAGCGGCAGAAAGAGCTGGAGAGCAAT
TACCGCCGGGTGTGGGGCTCTCCAGGTGGGGAGGGCACAGGGGACCTGGACGAATTTGACTTCTGAGA
CCAACACTACACTTGACCCTTCACGGAATCCAGACTCTTCCTGGACTGGCTTGCCTCCTCCCCACCTC
CCCACCCTGGAACCCCTGAGGGCCAAACAGCAGAGTGGAGCTGAGCTGTGGACCTCTCGGGCAACTCT
GTGGGTGTGGGGGCCCTGGGTGAATGCTGCTGCCCCTGCTGGCAGCCACCTTGAGACCTCACCGGGCC
TGTGATATTTGCTCTCCTGAACTCTCACTCAATCCTCTTCCTCTCCTCTGTGGCTTTTCCTGTTATTG
TCCCCTAATGATAGGATATTCCCTGCTGCCTACCTGGAGATTCAGTAGGATCTTTTGAGTGGAGGTGG
GTAGAGAGAGCAAGGAGGGCAGGACACTTAGCAGGCACTGAGCAAGCAGGCCCCCACCTGCCCTTAGT
GATGTTTGGAGTCGTTTTACCCTCTTCTATGGAATTGCCTGTGGATTCCTTCTCCCTTCCCTGCCCAC
CGTGTCCTACAATTGTGCTCTGAGTGAGAGCCTTCCTCTCTGCTAGGAAGGTTATGTGCCTTACTCCG
CAATCGGAAAGTTAGCCACGTTCTAATCGTTATACAAGGCTAAAAAAAATAAATATTTATACCCGTTT
TTCCCTGATTTATTTTTAAATATTATATTATTTTTAATATAATTTGTGGG

NOV41i, CG56878-04

| | | | |
|---|---|---|---|
| Protein Sequence | SEQ ID NO: 966 | 650 aa | MW at 73601.9kD |

MAAETLLSSLLGLLLLGLLLPASLTGGVGSLNLEELSEMRYGIEILPLPVMGGQSQSSDVVIVSSKYK
QRYECRLPAGAIHFQREREEETPAYQGPGIPELLSPMRDAPCLLKTKDWWTYEFCYGRHIQQYHMEDS
EIKGEVLYLGYYQSAFDWDDETAKASKQHRLKRYHSQTYGNGSKCDLNGRPREAEVRFLCDEGAGISG
DYIDRVDEPLSCSYVLTIRTPRLCPHPLLRPPPSAAPQAILCHPSLQPEEYMAYVQRQADSKQYGDKI
IEELQDLGPQVWSETKSGVAPQKMAGASPTKDDSKDSDFWKMLNEPEDQAPGGEEVPAEEQDPSPEAA
DSASGAPNDFQNNVQVKVIRSPADLIRFIEELKGGTKKGKPNIGQEQPVDDAAEVPQREPEKERGDPE
RQREMEEEEDEDEDKMRMNGSYWGEFEKELEGILLPSDRDRLRSETEKELDPDGLKKESERDRAMLAL
TSTLNKLIKRLEEKQSPELVKKHKKKRVVPKKPPPSPQPTEEDPEHRVRVRVTKLRLGGPNQDLTVLE
MKRENPQLKQIEGLVKELLEREGLTAAGKIEIKIVRPWAEGTEEGARWLTDEDTRNLKEIFFNILVPG
AEEAQKERQRQKELESNYRRVWGSPGGEGTGDLDEFDF

| | |
|---|---|
| SEQ ID NO: 967 | 2739 bp |

| | | |
|---|---|---|
| NOV41j, SNP13382511 of | ORF Start: ATG at 86 | ORF Stop: TGA at 2090 |

| | | |
|---|---|---|
| CG56878-01, DNA Sequence | SNP Pos: 2596 | SNP Change: C to T |

TTGCACTCTCCCACACCCTTTTCTTTTCGTCCGCTCTTCGCTTATTTCTCCCGCCGTCTCCTCTGCAT
AAGAAGGGGAACGAAAGATGGCGGCGGAAACGCTGCTGTCCAGTTTGTTAGGACTGCTGCTTCTGGGA
CTCCTGTTACCCGCAAGTCTGACCGGCGGTGTCGGGAGCCTGAACCTGGAGGAGCTGAGTGAGATGCG
TTATGGGATCGAGATCCTGCCGTTGCCTGTCATGGGAGGGCAGAGCCAATCTTCGGACGTGGTGATTG
TCTCCTCTAAGTACAAACAGCGCTATGAGTGTCGCCTGCCAGCTGGAGCTATTCACTTCCAGCGTGAA
AGGGAGGAGGAAACACCTGCTTACCAAGGGCCTGGGATCCCTGAGTTGTTGAGCCCAATGAGAGATGC
TCCCTGCTTGCTGAAGACAAAGGACTGGTGGACATATGAATTCTGTTATGGACGCCACATCCAGCAAT
ACCACATGGAAGATTCAGAGATCAAAGGTGAAGTCCTCTATCTCGGCTACTACCAATCAGCCTTCGAC
TGGGATGATGAAACAGCCAAGGCCTCCAAGCAGCATCGTCTTAAACGCTACCACAGCCAGACCTATGG
CAATGGGTCCAAGTGCGACCTTAATGGGAGGCCCCGGGAGGCCGAGGTTCGGTTCCTCTGTGACGAGG
GTGCAGGTATCTCTGGGGACTACATCGATCGCGTGGACGAGCCCTTGTCCTGCTCTTATGTGCTGACC
ATTCGCACTCCTCGGCTCTGCCCCCACCCTCTCCTCCGGCCCCCACCCAGTGCTGCACCGCAGGCCAT
CCTCTGTCACCCTTCCCTACAGCCTGAGGAGTACATGGCCTACGTTCAGAGGCAAGCCGTAGACTCAA
AGCAGTATGGAGATAAAATCATAGAGGAGCTGCAAGATCTAGGCCCCCAAGTGTGGAGTGAGACCAAG
TCTGGGGTGGCACCCCAAAAGATGGCAGGTGCGAGCCCGACCAAGGATGACAGTAAGGACTCAGATTT
CTGGAAGATGCTTAATGAGCCAGAGGACCAGGCCCCAGGAGGGGAGGAGGTGCCGGCTGAGGAGCAGG
ACCCAAGCCCTGAGGCAGCAGATTCAGCTTCTGGTGCTCCCAATGATTTTCAGAACAACGTGCAGGTC
AAAGTCATTCGAAGCCCTGCGGATTTGATTCGATTCATAGAGGAGCTGAAAGGTGGAACAAAAAAGGG
GAAGCCAAATATAGGCCAAGAGCAGCCTGTGGATGATGCTGCAGAAGTCCCTCAGAGGGAACCAGAGA
AGGAAAGGGGTGATCCAGAACGGCAGAGAGAGATGGAAGAAGAGGAGGATGAGGATGAGGATGAGGAT
GAAGATGAGGATGAACGGCAGTTACTGGGAGAATTTGAGAAGGAACTGGAAGGGATCCTGCTTCCGTC
AGACCGAGACCGGCTCCGTTCGGAGGTGAAGGCTGGCATGGAGCGGGAACTGGAGAACATCATCCAGG
AGACAGAGAAAGAGCTGGACCCAGATGGGCTGAAGAAGGAGTCAGAGCGGGATCGGGCAATGCTGGCT
CTCACATCCACTCTCAACAAACTCATCAAAAGACTGGAGGAAAAACAGAGTCCAGAGCTGGTGAAGAA
GCACAAGAAAAAGAGGGTTGTCCCCAAAAAGCCTCCCCCATCACCCCAACCTACAGAGGAGGATCCTG
AGCACAGAGTCCGGGTCCGGGTCACCAAGCTCCGTCTCGGAGGCCCTAATCAGGATCTGACTGTCCTC
GAGATGAAACGGGAAAACCCACAGCTGAAACAAATCGAGGGGCTGGTGAAGGAGCTGCTGGAGAGGGA
GGGACTCACAGCTGCAGGGAAAATTGAGATCAAAATTGTCCGCCCATGGGCTGAAGGGACTGAAGAGG
GGAGCTGAAGAGGCCCAGAAGGAACGCCAGCGGCAGAAAGAGCTGGAGAGCAATTACCGCCGGGTGTG
GGGCTCTCCAGGTGGGGAGGGCACAGGGGACCTGGACGAATTTGACTTCTGAGACCAACACTACACTT
GACCCTTCACGGAATCCAGACTCTTCCTGGACTGGCTTGCCTCCTCCCCACCTCCCCACCCTGGAACC
CCTGAGGGCCAAACAGCAGAGTGGAGCTGAGCTGTGGACCTCTCGGGCAACTCTGTGGGTGTGGGGGC
CCTGGGTGAATGCTGCTGCCCCTGCTGGCAGCCACCTTGAGACCTCACCGGGCCTGTGATATTTGCTC
TCCTGAACTCTCACTCAATCCTCTTCCTCTCCTCTGTGGCTTTTCCTGTTATTGTCCCCTAATGATAC
GATATTCCCTGCTGCCTACCTGGAGATTCAGTAGGATCTTTTGAGTGGAGGTGGGTAGAGAGAGCAAG
GAGGGCAGGACACTTAGCAGGCACTGAGCAAGCAGGCCCCCACCTGCCCTTAGTGATGTTTGGAGTCG
TTTTACCCTCTTCTATTGAATTGCCTTGGGATTTCCTTCTCCCTTTCCCTGCCCACCCTGTCCCCTAC
AATTTGTGCTTTTGAGTTGAGGAGCCTTCACCTCTGTTGCTGAGGAAATGGTAGAATGCTGCCTATCA
CCTCCAGCACAATCCCAGCGACAAAGGTGTGAAGCACCCACCATGTTCTTGAACAATCAGGTTTCTAA
ATAAACAACTGGACCATCA

| | |
|---|---|
| NOV41j, SNP13382511 of | MW at 75659.9kD |

| | | | |
|---|---|---|---|
| CG56878-01 | SEQ ID NO: 968 | 668 aa | SNP Change: no change |

MAAETLLSSLLGLLLLGLLLPASLTGGVGSLNLEELSEMRYGIEILPLPVMGGQSQSSDVVIVSSKYK
QRYECRLPAGAIHFQREREEETPAYQGPGIPELLSPMRDAPCLLKTKDWWTYEFCYGRHIQQYHMEDS
EIKGEVLYLGYYQSAFDWDDETAKASKQHRLKRYHSQTYGNGSKCDLNGRPREAEVRFLCDEGAGISG
DYIDRVDEPLSCSYVLTIRTPRLCPHPLLRPPPSAAPQAILCHPSLQPEEYMAYVQRQAVDSKQYGDK
IIEELQDLGPQVWSETKSGVAPQKMAGASPTKDDSKDSDFWKMLNEPEDQAPGGEEVPAEEQDPSPEA
ADSASGAPNDFQNNVQVKVIRSPADLIRFIEELKGGTKKGKPNIGQEQPVDDAAEVPQREPEKERGDP
ERQREMEEEEDEDEDEDEDEDERQLLGEFEKELEGILLPSDRDRLRSEVKAGMERELENIIQETEKEL
DPDGLKKESERDRAMLALTSTLNKLIKRLEEKQSPELVKKHKKKRVVPKKPPPSPQPTEEDPEHRVRV
RVTKLRLGGPNQDLTVLEMKRENPQLKQIEGLVKELLEREGLTAAGKIEIKIVRPWAEGTEEGARWLT
DEDTRNLKEIFFNILVPGAEEAQKERQRQKELESNYRRVWGSPGGEGTGDLDEFDF

| | |
|---|---|
| SEQ ID NO: 969 | 2739 bp |

| | | |
|---|---|---|
| NOV41k, SNP13382506 of | ORF Start: ATG at 86 | ORF Stop: TGA at 2090 |

| | | |
|---|---|---|
| CG56878-01, DNA Sequence | SNP Pos: 2671 | SNP Change: C to T |

TTGCACTCTCCCACACCCTTTTCTTTTCGTCCGCTCTTCGCTTATTTCTCCCGCCGTCTCCTCTGCAT
CTCCTGTTACCCGCAAGTCTGACCGGCGGTGTCGGGAGCCTGAACCTGGAGGAGCTGAGTGAGATGCG
TTATGGGATCGAGATCCTGCCGTTGCCTGTCATGGGAGGGCAGAGCCAATCTTCGGACGTGGTGATTG
AGGGAGGAGGAAACACCTGCTTACCAAGGGCCTGGGATCCCTGAGTTGTTGAGCCCAATGAGAGATGC
TCCCTGCTTGCTGAAGACAAAGGACTGGTGGACATATGAATTCTGTTATGGACGCCACATCCAGCAAT
ACCACATGGAAGATTCAGAGATCAAAGGTGAAGTCCTCTATCTCGGCTACTACCAATCAGCCTTCGAC
TGGGATGATGAAACAGCCAAGGCCTCCAAGCAGCATCGTCTTAAACGCTACCACAGCCAGACCTATGG
CAATGGGTCCAAGTGCGACCTTAATGGGAGGCCCCGGGAGGCCGAGGTTCGGTTCCTCTGTGACGAGG
GTGCAGGTATCTCTGGGGACTACATCGATCGCGTGGACGAGCCCTTGTCCTGCTCTTATGTGCTGACC
ATTCGCACTCCTCGGCTCTGCCCCCACCCTCTCCTCCGGCCCCCACCCAGTGCTGCACCGCAGGCCAT
CCTCTGTCACCCTTCCCTACAGCCTGAGGAGTACATGGCCTACGTTCAGAGGCAAGCCGTAGACTCAA
AGCAGTATGGAGATAAAATCATAGAGGAGCTGCAAGATCTAGGCCCCCAAGTGTGGAGTGAGACCAAG
TCTGGGGTGGCACCCCAAAAGATGGCAGGTGCGAGCCCGACCAAGGATGACAGTAAGGACTCAGATTT
CTGGAAGATGCTTAATGAGCCAGAGGACCAGGCCCCAGGAGGGGAGGAGGTGCCGGCTGAGGAGCAGG
ACCCAAGCCCTGAGGCAGCAGATTCAGCTTCTGGTGCTCCCAATGATTTTCAGAACAACGTGCAGGTC
AAAGTCATTCGAAGCCCTGCGGATTTGATTCGATTCATAGAGGAGCTGAAAGGTGGAACAAAAAAGGG
GAAGCCAAATATAGGCCAAGAGCAGCCTGTGGATGATGCTGCAGAAGTCCCTCAGAGGGAACCAGAGA
AGGAAAGGGGTGATCCAGAACGGCAGAGAGAGATGGAAGAAGAGGAGGATGAGGATGAGGATGAGGAT
GAAGATGAGGATGAACGGCAGTTACTGGGAGAATTTGAGAAGGAACTGGAAGGGATCCTGCTTCCGTC
AGACCGAGACCGGCTCCGTTCGGAGGTGAAGGCTGGCATGGAGCGGGAACTGGAGAACATCATCCAGG
AGACAGAGAAAGAGCTGGACCCAGATGGGCTGAAGAAGGAGTCAGAGCGGGATCGGGCAATGCTGGCT
CTCACATCCACTCTCAACAAACTCATCAAAAGACTGGAGGAAAAACAGAGTCCAGAGCTGGTGAAGAA
GCACAAGAAAAAGAGGGTTGTCCCCAAAAAGCCTCCCCCATCACCCCAACCTACAGAGGAGGATCCTG
AGCACAGAGTCCGGGTCCGGGTCACCAAGCTCCGTCTCGGAGGCCCTAATCAGGATCTGACTGTCCTC
GAGATGAAACGGGAAAACCCACAGCTGAAACAAATCGAGGGGCTGGTGAAGGAGCTGCTGGAGAGGGA
GGGACTCACAGCTGCAGGGAAAATTGAGATCAAAATTGTCCGCCCATGGGCTGAAGGGACTGAAGAGG
GTGCACGTTGGCTGACTGATGAGGACACGAGAAACCTCAAGGAGATCTTCTTCAATATCTTGGTGCCG
GGAGCTGAAGAGGCCCAGAAGGAACGCCAGCGGCAGAAAGAGCTGGAGAGCAATTACCGCCGGGTGTG
GGGCTCTCCAGGTGGGGAGGGCACAGGGGACCTGGACGAATTTGACTTCTG&GACCAACACTACACTT
CCTGAGGGCCAAACAGCAGAGTGGAGCTGAGCTGTGGACCTCTCGGGCAACTCTGTGGGTGTGGGGGC
CCTGGGTGAATGCTGCTGCCCCTGCTGGCAGCCACCTTGAGACCTCACCGGGCCTGTGATATTTGCTC
TCCTGAACTCTCACTCAATCCTCTTCCTCTCCTCTGTGGCTTTTCCTGTTATTGTCCCCTAATGATAG
GATATTCCCTGCTGCCTACCTGGAGATTCAGTAGGATCTTTTGAGTGGAGGTGGGTAGAGAGAGCAAG
GAGGGCAGGACACTTAGCAGGCACTGAGCAAGCAGGCCCCCACCTGCCCTTAGTGATGTTTGGAGTCG
TTTTACCCTCTTCTATTGAATTGCCTTGGGATTTCCTTCTCCCTTTCCCTGCCCACCCTGTCCCCTAC
CCTCCAGCACAATCCCAGTGAAAAAGGTGTGAAGCACCCACCATGTTCTTGAACAATCAGGTTTCTAA
ATAAACAACTGGACCATCA

| | |
|---|---|
| NOV41k, SNP13382506 of | MW at 75659.9kD |

| | | | |
|---|---|---|---|
| CG56878-01, Protein Sequence | SEQ ID NO: 970 | 668 aa | SNP Change: no change |

MAAETLLSSLLGLLLLGLLLPASLTGGVGSLNLEELSEMRYGIEILPLPVMGGQSQSSDVVIVSSKYK
QRYECRLPAGAIHFQREREEETPAYQGPGIPELLSPMRDAPCLLKTKDWWTYEFCYGRHIQQYHMEDS
EIKGEVLYLGYYQSAFDWDDETAKASKQHRLKRYHSQTYGNGSKCDLNGRPREAEVRFLCDEGAGISG
DYIDRVDEPLSCSYVLTIRTPRLCPHPLLRPPPSAAPQAILCHPSLQPEEYMAYVQRQAVDSKQYGDK
IIEELQDLGPQVWSETKSGVAPQKMAGASPTKDDSKDSDFWKMLNEPEDQAPGGEEVPAEEQDPSPEA
ADSASGAPNDFQNNVQVKVIRSPADLIRFIEELKGGTKKGKPNIGQEQPVDDAAEVPQREPEKERGDP
ERQREMEEEEDEDEDEDEDEDERQLLGEFEKELEGILLPSDRDRLRSEVKAGMERELENIIQETEKEL
DPDGLKKESERDRAMLALTSTLNKLIKRLEEKQSPELVKKHKKKRVVPKKPPPSPQPTEEDPEHRVRV
RVTKLRLGGPNQDLTVLEMKRENPQLKQIEGLVKELLEREGLTAAGKIEIKIVRPWAEGTEEGARWLT
DEDTRNLKEIFFNILVPGAEEAQKERQRQKELESNYRRVWGSPGGEGTGDLDEFDF

A ClustalW comparison of the above protein sequences yields the following sequence alignment shown in Table 41B.

TABLE 41B
Comparison of the NOV41 protein sequences.

| | | |
|---|---|---|
| NOV41a | MAAETLLSSLLGLLLLGLLLPASLTGGVGSLNLEELSEMRYGIEILPLPVMGGQSQSSDV | (SEQ ID NO:950) |
| NOV4lb | -------G----------------TGGVGSLNLEELSEMRYGIEILPLPVMGGQSQSSDV | (SEQ ID NO:952) |
| NOV41c | --SEFALG----------------TGGVGSLNLEELSEMRYGIEILPLPVMGGQSQSSDV | (SEQ ID NO:954) |
| NOV41d | -------G----------------TGGVGSLNLEELSEMRYGIEILPLPVMGGQSQSSDV | (SEQ ID NO:956) |
| NOV41e | -------G----------------TGGVGSLNLEELSEMRYGIEILPLPVMGGQSQSSDV | (SEQ ID NO:958) |
| NOV41f | -------G----------------TGGVGSLNLEELSEMRYGIEILPLPVMGGQSQSSDV | (SEQ ID NO:960) |
| NOV41g | -------G------------------GVGSLNLEELSEMRYGIEILPLPVMGGQSQSSDV | (SEQ ID NO:960) |
| NOV41h | -------G------------------GVGSLNLEELSEMRYGIEILPLPVMGGQSQSSDV | (SEQ ID NO:962) |
| NOV41i | MAAETLLSSLLGLLLLGLLLPASLTGGVGSLNLEELSEMRYGIEILPLPVMGGQSQSSDV | (SEQ ID NO:964) |
| NOV41a | VIVSSKYKQRYECRLPAGAIHFQREREEETPAYQGPGIPELLSPMRDAPCLLKTKDWWTY | (SEQ ID NO:966) |
| NOV41b | VIVSSKYKQRYECRLPAGAIHFQREREEETPAYQGPGIPELLSPMRDAPCLLKTKDWWTY | |
| NOV41c | VIVSSKYKQRYECRLPAGAIHFQREREEETPAYQGPGIPELLSPMRDAPCLLKTKDWWTY | |
| NOV4ld | VIVSSKYKQRYECRLPAGAIHFQREREEETPAYQGPGIPELLSPMRDAPCLLKTKDWWTY | |
| NOV41e | VIVSSKYKQRYECRLPAGAIHFQREREEETPAYQGPGIPELLSPMPDAPCLLKTKDWWTY | |
| NOV41f | VIVSSKYKQRYECRLPAGAIHFQREREEETPAYQGPGIPELLSPMRDAPCLLKTKDWWTY | |
| NOV41g | VIVSSKYKQRYECRLPAGAIHFQREREEETPAYQGPGIPELLSPMPDAPCLLKTKDWWTY | |
| NOV41h | VIVSSKYKQRYECRLPAGAIHFQREREEETPAYQGPGIPELLSPMRDAPCLLKTKDWWTY | |
| NOV41i | VIVSSKYKQRYECRLPAGAIHFQREREEETPAYQGPGIPELLSPMRDAPCLLKTKDWWTY | |
| NOV41a | EFCYGRHIQQYHMEDSEIKGEVLYLGYYQSAFDWDDETAKASKQHRLKRYHSQTYGNGSK | |
| NOV41b | EFCYGRHIQQYHMEDSEIKGEVLYLGYYQSAFDWDDETAKASKQHRLKRYHSQTYGNGSK | |
| NOV41c | EFCYGRHIQQYHMEDSEIKGEVLYLGYYQSAFDWDDETAKASKQHRLKRYHSQTYGNGSK | |
| NOV41d | EFCYGRHIQQYHMEDSEIKGEVLYLGYYQSAFDWDDETAKASKQHRLKRYHSQTYGNGSK | |
| NOV41e | EFCYGRHIQQYHMEDSEIKGEVLYLGYYQSAFDWDDETAKASKQHRLKRYHSQTYGNGSK | |
| NOV41f | EFCYGRHIQQYHMEDSEIKGEVLYLGYYQSAFDWDDETAKASKQHRLKRYHSQTYGNGSK | |
| NOV41g | EFCYGRHIQQYHMEDSEIKGEVLYLGYYQSAFDWDDETAKASKQHRLKRYHSQTYGNGSK | |
| NOV41h | EFCYGRHIQQYHMEDSEIKGEVLYLGYYQSAFDWDDETAKASKQHRLKRYHSQTYGNGSK | |
| NOV41i | EFCYGRHIQQYHMEDSEIKGEVLYLGYYQSAFDWDDETAKASKQHRLKRYHSQTYGNGSK | |
| NOV41a | CDLNGRPREAEVRFLCDEGAGISGDYIDRVDEPLSCSYVLTIRTPRLCPHPLLRPPPSAA | |
| NOV41b | CDLNGRPREAEVRFLCDEGAGISGDYIDRVDEPLSCSYVLTIRTPRLCPHPLLRPPPSAA | |
| NOV41c | CDLNGRPREAEVRFLCDEGAGISGDYIDRVDEPLSCSYVLTIRTPRLCPHPLLRPPPSAA | |
| NOV41d | CDLNGRPREAEVRFLCDEGAGISGDYIDRVDEPLSCSYVLTIRTPRLCPHPLLRPPPSAA | |
| NOV41e | CDLNGRPREAEVRFLCDEGAGISGDYIDRVDEPLSCSYVLTIRTPRLCPHPLLRPPPSAA | |
| NOV41f | CDLNGRPREAIEVRFLCDEGAGISGDYIDRVDEPLSCSYVLTIRTPRLCPHPLLRPPPSAA | |
| NOV41g | CDLNGRPREAEVRFLCDEGAGISGDYIDRVDEPLSCSYVLTIRTPRLCPHPLLRPPPSAA | |
| NOV41h | CDLNGRPREAEVRFLCDEGAGISGDYIDRVDEPLSCSYVLTIRTPRLCPHPLLRPPPSAA | |
| NOV41i | CDLNGRPREAEVRFLCDEGAGISGDYIDRVDEPLSCSYVLTIRTPRLCPHPLLRPPPSAA | |
| NOV41a | PQAILCHPSLQPEEYMAYVQRQAVDSKQYGDKIIEELQDLGPQVWSETKSGVAPQKMAGA | |
| NOV41b | PQAILCHPSLQPEEYMAYVQRQAD-SKQYGDKIIEELQDLGPQVWSETKSGVAPQKMAGA | |
| NOV41c | PQAILCHPSLQPEEYMAYVQRQAD-SKQYGDKIIEELQDLGPQVWSETKSGVAPQKMAGA | |
| NOV41d | PQAILCHPSLQPEEYMAYVQRQAD-SKQYGDKIIEELQDLGPQVWSETKSGVAPQKMAGA | |
| NOV41e | PQAILCHPSLQPEEYMAYVQRQAD-SKQYGDKIIEELQDLGPQVWSETKSGVAPQKMAGA | |
| NOV41f | PQAILCHPSLQPEEYMAYVQRQAD-SKQYGDKIIEELQDLGPQVWSETKSGVAPQKMAGA | |
| NOV41g | PQAILCHPSLQPEEYMAYVQRQAD-SKQYGDKIIEELQDLGPQVWSETKSGVAPQKMAGA | |
| NOV41h | PQAILCHPSLQPEEYMAYVQRQAD-SKQYGDKIIEELQDLGPQVWSETKSGVAPQKMAGA | |
| NOV41i | PQAILCHPSLQPEEYMAYVQRQAD-SKQYGDKIIEELQDLGPQVWSETKSGVAPQKMAGA | |
| NOV41a | SPTKDDSKDSDFWKMLNEPEDQAPGGEEVPAEEQDPSPEAADSASGAPNDFQNNVQVKVI | |
| NOV41b | SPTKDDSKDSDFWKMLNEPEDQAPGGEEVPAEEQDPSPEAADSASGAPNDFQNNVQVKVI | |
| NOV41c | SPTKDDSKDSDFWKMLNEPEDQAPGGEEVPAEEQDPSPEAADSASGAPNDFQNNVQVKVI | |
| NOV41d | SPTKDDSKDSDFWKMLNEPEDQAPGGEEVPAEEQDPSPEAADSASGAPNDFQNNVQVKVI | |
| NOV41e | SPTKDDSKDSDFWKMLNEPEDQAPGGEEVPAEEQDPSPEAADSASGAPNDFQNNVQVKVI | |
| NOV41f | SPTKDDSKDSDFWKMLNEPEDQAPGGEEVPAEEQDPSPEAADSASGAPNDFQNNVQVKVI | |
| NOV41g | SPTKDDSKDSDFWKMLNEPEDQAPGGEEVPAEEQDPSPEAADSASGAPNDFQNNVQVKVI | |
| NOV41h | SPTKDDSKDSDFWKMLNEPEDQAPGGEEVPAEEQDPSPEAADSASGAPNDFQNNVQVKVI | |
| NOV41i | SPTKDDSKDSDFWKMLNEPEDQAPGGEEVPAEEQDPSPEAADSASGAPNDFQNNVQVKVI | |
| NOV41a | RSPADLIRFIEELKGGTKKGKPNIGQEQPVDDAAEVPQREPEKERGDPERQREMEEEEDE | |
| NOV41b | RSPADLIRFIEELKGGTKKGKPNMDQEQPVDDAAEVPQREPEKERGDPERQREMEEEEDE | |
| NOV41c | RSPADLIRFIEELKGGTKKGKPNIGQEQPVDDAAEVPQREPEKERGDPERQREMEEEEDE | |
| NOV41d | RSPADLIRFIEELKGGIKKGKPNIGQEQPVDDAAEVPQREPEKERGDPERQREMEEEEDE | |
| NOV41e | RSPADLIRFIEELKGGTKKGKPNIGQEQPVDDAAEVPQREPEKERGDPERQREMEEEEDE | |
| NOV41f | RSPADLIRFIEELKGGTKKGKPNIGQEQPVDDAAEVPQREPEKERGDPERQREMEEEEDE | |
| NOV41g | RSPADLIRFIEELKGGTKKGKPNIGQEQPVDDAAEVPQREPEKERGDPERQREMEEEEDE | |
| NOV41h | RSPADLIRFIEELKGGIKKGKPNIGQEQPVDDAAEVPQREPEKERGDPERQREMEEEEDE | |
| NOV41i | RSPADLIRFIEELKGGTKKGKPNIGQEQPVDDAAEVPQREPEKERGDPERQREMEEEEDE | |
| NOV4la | DEDEDEDEDERQLLGEFEKELEGILLPSDRDRLRSEVKAGMERELENIIQETEKELDPDG | |
| NOV41b | DEDEDEDEDERQLLGEFEKELEGILLPSDRDRLRSEVKAGMERELENIIQETEKELDPDG | |
| NOV41c | DEDEDEDEDERQLLGEFEKELEGILLPSDRGRLRSEVKAGMERELENIIQETEKELDPDG | |
| NOV41d | DEDEDEDEDERQLLGEFEKELEGILLPSDRDRLRSEVKAGMERELENIIQETEKELDPDG | |
| NOV41e | DEDEDEDEDERQLLGEFEKELEGILLPSDRDRLRSEVKAGMERELENIIQETEKELDPDG | |
| NOV41f | DEDEDEDEDERQLLGEFEKELEGILLPSDRDRLRSEVKAGMERELENIIQETEKELDPDG | |
| NOV41g | DEDEDEDEDERQLLGEFEKELEGILLPSDRDRLRSEVKAGMERELENIIQETEKELDPDG | |
| NOV41h | DEDEDEDEDERQLLGEFEKELEGILLPSDRDRLRSEVKAGMERELENIIQETEKELDPDG | |
| NOV41i | DED--KMPMNGSYWGEFEKELEGILLPSDRDRLR-------S--------ETEKELDPDG | |
| NOV41a | LKKESERDRAMLALTSTLNKLIKRLEEKQSPELVKKHKKKRVVPKKPPPSPQPTEEDPEH | |
| NOV41b | LKKESERDRAMLALTSTLNKPIKRLEEKQSPELVKKHKKKRVVPKKPPPSPQPT------ | |
| NOV41c | LKKESERDHAMLALTSTLNKLIKRLEEKQSPELVKKHKKKRVVPKKPPPSPQPT------ | |
| NOV41d | LKKESERDRAMLALTSTLNKLIKRLEEKQSPELVKKHKKKRVVPKKPPPSPQPT------ | |
| NOV41e | LKKESERDRAMLALTSTLNKLIKRLEEKQSPELVKKHKKKRVVPKKPPPSPQPTEEDPEH | |
| NOV41f | LKKESERDRAMLALTSTLNKLIKRLEEKQSPELVKKHKKKRVVPKKPPPSPQPTEEDPEH | |
| NOV41g | LKKESERDRAMLALTSTLNKLIKRLEEKQSPELVKKHKKKRVVPKKPPPSPQPTEEDPEH | |
| NOV41h | LKKESERDRAMLALTSTLNKLIKRLEEKQSPELVKKHKKKRVVPKKPPPSPQPT------ | |
| NOV41i | LKKESERDRAMLALTSTLNKLIKRLEEKQSPELVKKHKKKRVVPKKPPPSPQPTEEDPEH | |
| NOV41a | RVRVRVTKLRLGGPNQDLTVLEMKRENPQLKQIEGLVKELLEREGLTAAGKIEIKIVRPW | |
| NOV41b | -------------------------------------------------GKIEIKIVRPW | |
| NOV41c | -------------------------------------------------GKIEIKIVRPW | |
| NOV41d | -------------------------------------------------GKIEIKIVRPW | |
| NOV41e | RVRVRVTKLRLGGPNQDLTVLEMKRENPQLKQIEGLVKELLEREGLTAAGKIEIKIVRPW | |
| NOV41f | RVRVRVTKLRLGGPNQDLTVLEMKRENPQLKQIEGLVKELLEREGLTAAGKIEIKIVRPW | |
| NOV41g | RVRVRVTKLRLGGPNQDLTVLEMKRENPQLKQIEGLVKELLEREGLTAAGKIEIKIVRPW | |
| NOV41h | -------------------------------------------------GKIEIKIVRPW | |
| NOV41i | RVRVRVTKLRLGGPNQDLTVLEMKRENPQLKQIEGLVKELLEREGLTAAGKIEIKIVRPW | |
| NOV41a | AEGTEEGARWLTDEDTRNLKEIFFNILVPGAEEAQKERQRQKELESNYRRVWGSPGGEGT | |
| NOV41b | AEGTEEGARWLTDEDTRNLKEIFFNILVPGAEEAQKERQRQKELESNYRRVWGSPGGEGT | |
| NOV41c | AEGTEEGARWLTDEDTRNLKEIFFNILVPGAEEAQKERQRQKELESNYRRVWGSPGGEGT | |
| NOV41d | AEGTEEGARWLTDEDTRNLKEIFFNILVPGAEEAQKERQRQKELESNYRRVWGSPGGEGT | |
| NOV41e | AEGTEEGARWLTDEDTRNLKEIFFNILVPGAEEAQKERQRQKELESNYRRVWGSPGGEGT | |
| NOV41f | AEGTEEGARWLTDEDTRNLKEIFFNILVPGAEEAQKERQRQKELESNYRRVWGSPGGEGT | |
| NOV41g | AEGTEEGARWLTDEDTRNLKEIFFNILVPGAEEAQKERQRQKELESNYRRVWGSPGGEGT | |
| NOV41h | AEGTEEGARWLTDEDTRNLKEIFFNILVPGAEEAQKERQRQKELESNYRRVWGSPGGEGT | |
| NOV41i | AEGTEEGARWLTDEDTRNLKEIFFNILVPGAEEAQKERQRQKELESNYRRVWGSPGGEGT | |
| NOV41a | GDLDEFDF-- | |
| NOV41b | GDLDEFDFVD | |
| NOV41c | GDLDEFDFVD | |
| NOV41d | GDLDEFDFVD | |
| NOV41e | GDLDEFDFVD | |
| NOV41f | GDLDEFDFVD | |
| NOV41g | GDLDEFDF-- | |
| NOV41h | GDLDEFDF-- | |
| NOV41i | GDLDEFDF-- | |

Further analysis of the NOV41a protein yielded the following properties shown in Table 41C.

TABLE 41C
Protein Sequence Properties NOV41a

| | |
|---|---|
| SignalP analysis: | Cleavage site between residues 26 and 27 |
| PSORT II analysis: | |

| PSG: | a new signal peptide prediction method | |
|---|---|---|
| | N-region: | length 4; pos. chg 0; neg. chg 1 |
| | H-region: | length 29; peak value 0.00 |
| | PSG score: | −4.40 |

| | |
|---|---|
| GvH: | von Heijne's method for signal seq. recognition |
| | GvH score (threshold: −2.1): 3.65 |
| | possible cleavage site: between 25 and 26 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 1 | | |
| | Number of TMS(s) for threshold 0.5: 1 | | |
| | INTEGRAL | Likelihood = | Transmembrane 6-22 |
| | | −6.74 | |

| | |
|---|---|
| PERIPHERAL | Likelihood = 8.49 (at 48) |

| | |
|---|---|
| | ALOM score: −6.74 (number of TMSs: 1) |
| MTOP: | Prediction of membrane topology (Hartmann et al.) |
| | Center position for calculation: 13 |
| | Charge difference: −3.0 C(−3.0)-N(0.0) |
| | N >= C: N-terminal side will be inside |

>>> membrane topology: type 2 (cytoplasmic tail 1 to 6)

| MITDISC: | discrimination of mitochondrial targeting seq | | | |
|---|---|---|---|---|
| | R content: | 0 | Hyd Moment(75): | 8.03 |
| | Hyd Moment(95): | 5.55 | G content: | 5 |
| | D/E content: | 2 | S/T content: | 6 |

| | |
|---|---|
| | Score: −7.42 |
| Gavel: | prediction of cleavage sites for mitochondrial preseq |
| | cleavage site motif not found |

NUCDISC: discrimination of nuclear localization signals

| | |
|---|---|
| | pat4: KKHK (3) at 515 |
| | pat4: KHKK (3) at 516 |
| | pat4: HKKK (3) at 517 |
| | pat4: KKKR (5) at 518 |
| | pat7: none |
| | bipartite: KRLEEKQSPELVKKHKK at 503 |
| | content of basic residues: 13.6% |
| | NLS Score: 1.06 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 76.7 |
| COIL: | Lupas's algorithm to detect coiled-coil regions |
| 408 E | 0.52 |
| 409 R | 0.52 |
| 410 Q | 0.52 |
| 411 R | 0.52 |
| 412 E | 0.52 |
| 413 M | 0.74 |
| 414 E | 0.97 |
| 415 E | 0.97 |
| 416 E | 0.97 |
| 417 E | 0.97 |
| 418 D | 0.97 |
| 419 E | 0.97 |
| 420 D | 0.97 |
| 421 E | 0.97 |
| 422 D | 0.97 |
| 423 E | 0.97 |
| 424 D | 0.97 |
| 425 E | 0.97 |
| 426 D | 0.97 |
| 427 E | 0.97 |
| 428 D | 0.97 |
| 429 E | 0.97 |
| 430 R | 0.97 |
| 431 Q | 0.97 |
| 432 L | 0.97 |
| 433 L | 0.97 |
| 434 G | 0.97 |
| 435 E | 0.97 |
| 436 F | 0.97 |
| 437 E | 0.97 |
| 438 K | 0.97 |
| 439 E | 0.97 |
| 440 L | 0.97 |
| 441 E | 0.97 |
| 442 G | 0.84 |
| 443 I | 0.82 |
| | total: 36 residues |

Final Results (k = 9/23):

```
43.5%: nuclear
26.1%: mitochondrial
  8.7%: cytoplasmic
  4.3%: extracellular, including cell wall
  4.3%: Golgi
  4.3%: plasma membrane
  4.3%: vesicles of secretory system
  4.3%: peroxisomal
```

>> prediction for CG56878-01 is nuc (k = 23)

A search of the NOV41a protein against the Geneseq database, a proprietary database that contains sequences published in patents and patent publication, yielded several homologous proteins shown in Table 41D.

TABLE 41D
Geneseq Results for NOV41a

| | | NOV41a | Identities/ | | |
|---|---|---|---|---|---|
| | | Residues/ | Similarities for | | |
| Geneseq | Protein/Organism/Length | Match | the Matched | | Expect |
| Identifier | [Patent #, Date] | Residues | Region | | Value |
| ABP42584 | Human ovarian antigen HOFNF76, | 3 . . . 120 | 111/118 | (94%) | 8e−59 |
| | SEQ ID NO: 3716 - Homo sapiens, 140 | 6 . . . 123 | 115/118 | (97%) | |
| | aa. [WO200200677-A1 , 03-JAN-2002] | | | | |
| AAG76089 | Human colon cancer antigen protein | 508 . . . 668 | 100/161 | (62%) | 7e−45 |
| | SEQ ID NO: 6853 - Homo sapiens, 106 | 1 . . . 106 | 102/161 | (63%) | |
| | aa. [WO200122920-A2, 05-APR-2001] | | | | |
| ABP42294 | Human ovarian antigen HNHBC18, | 454 . . . 534 | 81/81 | (100%) | 5e−39 |
| | SEQ ID NO: 3426 - Homo sapiens, 93 | 1 . . . 81 | 81/81 | (100%) | |
| | aa. [WO200200677-A1, 03-JAN-2002] | | | | |
| AAG41826 | Arabidopsis thaliana protein fragment | 19 . . . 265 | 83/263 | (31%) | 1e−24 |
| | SEQ ID NO: 52087 - Arabidopsis | 26 . . . 273 | 124/263 | (46%) | |
| | thaliana, 282 aa. [EP1033405-A2, | | | | |
| | 06-SEP-2000] | | | | |
| AAG41828 | Arabidopsis thaliana protein fragment | 100 . . . 265 | 61/169 | (36%) | 6e−23 |
| | SEQ ID NO: 52089 - Arabidopsis | 48 . . . 208 | 90/169 | (53%) | |
| | thaliana, 217 aa. [EP1033405-A2, | | | | |
| | 06-SEP-2000] | | | | |

In a BLAST search of public sequence databases, the NOV41a protein was found to have homology to the proteins shown in the BLASTP data in Table 41E.

TABLE 41E
Public BLASTP Results for NOV41a

| | | NOV41a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q13438 | Protein OS-9 precursor - Homo | 1 . . . 668 | 667/668 (99%) | 0.0 |
| | sapiens (Human), 667 aa. | 1 . . . 667 | 667/668 (99%) | |
| Q8IZ58 | Hypothetical protein - Homo sapiens | 1 . . . 668 | 603/668 (90%) | 0.0 |
| | (Human), 615 aa (fragment). | 4 . . . 615 | 609/668 (90%) | |
| Q9BW99 | Similar to amplified in osteosarcoma - | 1 . . . 668 | 603/668 (90%) | 0.0 |
| | Homo sapiens (Human), 612 aa. | 1 . . . 612 | 609/668 (90%) | |
| Q8K2C7 | Hypothetical protein - Mus musculus | 1 . . . 668 | 489/676 (72%) | 0.0 |
| | (Mouse), 617 aa. | 1 . . . 617 | 544/676 (80%) | |
| Q922S5 | Unknown (Protein for | 47 . . . 668 | 447/630 (70%) | 0.0 |
| | IMAGE: 3598453) - Mus musculus | 1 . . . 571 | 501/630 (78%) | |
| | (Mouse), 571 aa (fragment). | | | |

PFam analysis predicts that the NOV41a protein contains the domains shown in the Table 41F.

TABLE 41F
Domain Analysis of NOV41a

| | | | |
|---|---|---|---|
| Pfam Domain | NOV41a | Identities/ | Expect |
| | Match Region | Similarities | Value |
| | | for the | |
| | | Matched Region | |

### Example 42

The NOV42 clone was analyzed, and the nucleotide and encoded polypeptide sequences are shown in Table 42A.

TABLE 42A
NOV42 Sequence Analysis

| | | |
|---|---|---|
| NOV42a, CG56904-01 | SEQ ID NO:971 | 1311 bp |
| DNA Sequence | ORF Start: ATG at 19 | ORF Stop: TAA at 1282 |

GGAGCTCCCACACTTTCAATGGGGAGGCCCACCCAGTGGCCGAGCCTGCTGCTGCTCCTGCTGTTGCC
GGGGCCCCCGCCCGTCGCCGGCTTGGAAGACGCTGCCTTCCCCCACCTGGGGGAGAGCTTGCAGCCCC
TGCCCCGGGCCTGTCCCCTGCGCTGCTCCTGCCCCCGAGTCGACACTGTGGACTGTGATGGCTTGGAC
CTTCGAGTGTTCCCGGACAACATCACCAGAGCCGCTCAGCACCTCTCCCTGCAGAACAACCAGCTCCA
GGAACTCCCCTACAATGAGCTGTCCCGCCTCAGTGGCCTGCGAACCCTCAACCTCCACAACAACCTCA
TCTCCTCCGAAGGCCTGCCTGACGAGGCCTTCGAGTCCCTCACCCAGCTGCAGCACCTCTGCGTGGCT
CACAACAAGAACAATCTCATCTCCAAGGTGCCCCGAGGAGCCCTGAGCCGCCAGACTCAACTCCGTGA
GCTCTACCTCCAGCACAACCAGCTGACAGACAGTGGCCTGGATGCCACCACCTTCAGCAAGCTGCATA
GCCTTGAATACCTGGATCTCTCCCACAACCAGCTGACCACAGTGCCCGCCGGCCTGCCCCGGACCCTG
GCTATCCTGCACCTGGGCCGCAACCGCATCCGGCAGGTGGAGGCGGCTCGGCTGCACGGGGCGCGTGG
TCTGCGCTATTTGTTGCTGCAGCACAACCAGCTGGGGAGCTCAGGGCTGCCCGCCGGGGCTCTGCGGC
CGCTGCGGGGCCTGCACACGCTGCACCTCGATGGCAATGGGCTGGACCGCGTGCCTCCAGCCCTGCCC
CGCCGCCTGCGTGCCCTGGTGCTGCCCCACAACCACGTGGCCGCGCTGGGTGCCCGTGACCTGGTCGC
CACACCGGGCCTGACGGAGCTTAACCTGGCCTATAACCGCCTGGCCAGCGCCCGTGTGCACCACCGGG
CCTTCCGCCGGTTGCGTGCCCTGCGCAGCCTCGACCTGGCAGGGAATCAGCTAACCCGGCTGCCCATG
GGCCTGCCCACTGGCCTGCGCACCCTGCAGCTGCAACGCAACCAGCTGCGGATGCTCGAGCCCGAGCC
TCTGGCCGGCCTGGACCAACTGCGGGAGCTCAGCCTGGCGCACAACCGGCTCCGGGTCGGCGACATCG
GGCCAGGCACCTGGCATGAGCTCCAAGCCCTCCAGGTCAGGCACAGGCTGGTTAGCCACACTGTCCCC
AGGGCCCCTCCATCCCCCTGCCTGCCCTGCCACGTCCCAAACATTCTAGTTAGCTGGTAAAGCAATCA
GAACAAGAAAATGATAAGA

| | | | |
|---|---|---|---|
| NOV42a, CG56904-01 | SEQ ID NO:972 | 421 aa | MW at 46667.3 kD |
| Protein Sequence | | | |

MGRPTQWPSLLLLLLLPGPPPVAGLEDAAFPHLGESLQPLPRACPLRCSCPRVDTVDCDGLDLRVFPD
NITRAAQHLSLQNNQLQELPYNELSRLSGLRTLNLHNNLISSEGLPDEAFESLTQLQHLCVAHNKNNL
ISKVPRGALSRQTQLRELYLQHNQLTDSGLDATTFSKLHSLEYLDLSHNQLTTVPAGLPRTLAILHLG
RNRIRQVEAARLHGARGLRYLLLQHNQLGSSGLPAGALRPLRGLHTLHLDGNGLDRVPPALPRRLRAL
VLPHNHVAALGARDLVATPGLTELNLAYNRLASARVHHRAFRRLPALRSLDLAGNQLTRLPMGLPTGL
RTLQLQRNQLRMLEPEPLAGLDQLRELSLAHNRLRVGDIGPGTWHELQALQVRHRLVSHTVPRAPPSP
CLPCHVPNILVSW

| | | |
|---|---|---|
| NOV42b, 272355087 | SEQ ID NO:973 | 1278 bp |
| DNA Sequence | ORF Start: at 1 | ORF Stop: end of sequence |

GGATCCACCATGGGGAGGCCCACCCAGTGGCCGAGCCTGCTGCTGCTCCTGCTGTTGCCGGGGCCCCC
GCCCGTCGCCGGCTTGGAAGACGCTGCCTTCCCCCACCTGGGGGAGAGCTTGCAGCCCCTGCCCCGGG
CCTGTCCCCTGCGCTGCTCCTGCCCCCGAGTCGACACTGTGGACTGTGATGGCTTGGACCTTCGAGTG
TTCCCGGACAACATCACCAGAGCCGCTCAGCACCTCTCCCTGCAGAACAACCAGCTCCAGGAACTCCC
CTACAATGAGCTGTCCCGCCTCAGTGGCCTGCGAACCCTCAACCTCCACAACAACCTCATCTCCTCCG
AAGGCCTGCCTGACGAGGCCTTCGAGTCCCTCACCCAGCTGCAGCACCTCTGCGTGGCTCACAACAAG
AACAATCTCATCTCCAAGGTGCCCCGAGGAGCCCTGAGCCGCCAGACTCAACTCCGTGAGCTCTACCT
CCAGCACAACCAGCTGACAGACAGTGGCCTGGATGCCACCACCTTCAGCAAGCTGCATAGCCTTGAAT
ACCTGGATCTCTCCCACAACCAGCTGACCACAGTGCCCGCCGGCCTGCCCCGGACCCTGGCTATCCTG
CACCTGGGCCGCAACCGCATCCGGCAGGTGGAGGCGGCTCGGCTGCACGGGGCGCGTGGTCTGCGCTA
TTTGTTGCTGCAGCACAACCAGCTGGGGAGCTCAGGGCTGCCCGCCGGGGCTCTGCGGCCGCTGCGGG
GCCTGCACACGCTGCACCTCTATGGCAATGGGCTGGACCGCGTGCCTCCAGCCCTGCCCCGCCGCCTG
CGTGCCCTGGTGCTGCCCCACAACCACGTGGCCGCGCTGGGTGCCCGTGACCTGGTCGCCACACCGGG
CCTGACGGAGCTTAACCTGGCCTATAACCGCCTGGCCAGCGCCCGTGTGCACCACCGGGCCTTCCGCC
GGTTGCGTGCCCTGCGCAGCCTCGACCTGGCAGGGAATCAGCTAACCCGGCTGCCCATGGGCCTGCCC
ACTGGCCTGCGCACCCTGCAGCTGCAACGCAACCAGCTGCGGATGCTCGAGCCCGAGCCTCTGGCCGG
CCTGGACCAACTGCGGGAGCTCAGCCTGGCGCACAACCGGCTCCGGGTCGGCGACATCGGGCCAGGCA
CCTGGCATGAGCTCCAAGCCCTCCAGGTCAGGCACAGGCTGGTTAGCCACACTGTCCCCAGGGCCCCT
CCATCCCCCTGCCTGCCCTGCCACGTCCCAAACATTCTAGTTAGCTGGGAATTC

| | | | |
|---|---|---|---|
| NOV42b, 272355087 | SEQ ID NO:974 | 426 aa | MW at 47236.9 kD |
| Protein Sequence | | | |

GSTMGRPTQWPSLLLLLLLPGPPPVAGLEDAAFPHLGESLQPLPRACPLRCSCPRVDTVDCDGLDLRV
FPDNITRAAQHLSLQNNQLQELPYNELSRLSGLRTLNLHNNLISSEGLPDEAFESLTQLQHLCVAHNK
NNLISKVPRGALSRQTQLRELYLQHNQLTDSGLDATTFSKLHSLEYLDLSHNQLTTVPAGLPRTLAIL
HLGRNRIRQVEAARLHGARGLRYLLLQHNQLGSSGLPAGALRPLRGLHTLHLYGNGLDRVPPALPRRL
RALVLPHNHVAALGARDLVATPGLTELNLAYNRLASARVHHRAFRRLRALRSLDLAGNQLTRLPMGLP
TGLRTLQLQRNQLRMLEPEPLAGLDQLRELSLAHNRLRVGDIGPGTWHELQALQVRHRLVSHTVPRAP
PSPCLPCHVPNILVSWEF

| | | |
|---|---|---|
| NOV42c, 246862506 | SEQ ID NO:975 | 1122 bp |
| DNA Sequence | ORF Start: at 3 | ORF Stop: at 1122 |

GGATCCACCATGGCTTGGACCTTCGAGTGTTCCCGGACAACATCACCAGAGCCGCTCAGCACCTCTCC
CTGCAGAACAACCAGCTCCAGGAACTCCCCTACAATGAGCTGTCCCGCCTCAGTGGCCTGCGAACCCT
CAACCTCCACAACAACCTCATCTCCTCCGAAGGCCTGCCTGACGAGGCCTTCGAGTCCCTCACCCAGC
TGCAGCACCTCTGCGTGGCTCACAACAAGAACAATCTCATCTCCAAGGTGCCCCGAGGAGCCCTGAGC
CGCCAGACTCAACTCCGTGAGCTCTACCTCCAGCACAACCAGCTGACAGACAGTGGCCTGGATGCCAC
CACCTTCAGCAAGCTGCATAGCCTTGAATACCTGGATCTCTCCCACAACCAGCTGACCACAGTGCCCG
CCGGCCTGCCCCGGACCCTGGCTATCCTGCACCTGGGCCGCAACCGCATCCGGCAGGTGGAGGCGGCT
CGGCTGCACGGGGCGCGTGGTCTGCGCTATTTGTTGCTGCAGCACAACCAGCTGGGGAGCTCAGGGCT
GCCCGCCGGGGCTCTGCGGCCGCTGCGGGGCCTGCACACGCTGCACCTCTATGGCAATGGGCTGGACC
GCGTGCCTCCAGCCCTGCCCCGCCGCCTGCGTGCCCTGGTGCTGCCCCACAACCACGTGGCCGCGCTG
GGTGCCCGTGACCTGGTCGCCACACCGGGCCTGACGGAGCTTAACCTGGCCTATAACCGCCTGGCCAG
CGCCCGTGTGCACCACCGGGCCTTCCGCCGGTTGCGTGCCCTGCGCAGCCTCGACCTGGCAGGGAATC
AGCTAACCCGGCTGCCCATGGGCCTGCCCACTGGCCTGCGCACCCTGCAGCTGCAACGCAACCAGCTG
CGGATGCTCGAGCCCGAGCCTCTGGCCGGCCTGGACCAACTGCGGGAGCTCAGCCTGGCGCACAACCG
GCTCCGGGTCGGCGACATCGGGCCAGGCACCTGGCATGAGCTCCAAGCCCTCCAGGTCAGGCACAGGC
TGGTTAGCCACACTGTCCCCAGGGCCCCTCCATCCCCCTGCCTGCCCTGCCACGTCCCAAACATTCTA
GTTAGCTGGGAATTCAAGGGCGAATTCCAAGCAC

| | | | |
|---|---|---|---|
| NOV42c, 246862506 | SEQ ID NO:976 | 373 aa | MW at 41726.5 kD |
| Protein Sequence | | | |

IHHGLDLRVFPDNITRAAQHLSLQNNQLQELPYNELSRLSGLRTLNLHNNLISSEGLPDEAFESLTQL
QHLCVAHNKNNLISKVPRGALSRQTQLRELYLQHNQLTDSGLDATTFSKLHSLEYLDLSHNQLTTVPA
GLPRTLAILHLGRNRIRQVEAARLHGARGLRYLLLQHNQLGSSGLPAGALRPLRGLHTLHLYGNGLDR
VPPALPRRLRALVLPHNHVAALGARDLVATPGLTELNLAYNRLASARVHHRAFRRLRALRSLDLAGNQ
LTRLPMGLPTGLRTLQLQRNQLRMLEPEPLAGLDQLRELSLAHNRLRVGDIGPGTWHELQALQVRHRL
VSHTVPRAPPSPCLPCHVPNILVSWEFKGEFQA

| | | |
|---|---|---|
| NOV42d, CG56904-04 | SEQ ID NO:977 | 1977 bp |
| DNA Sequence | ORF Start: ATG at 1 | ORF Stop: TGA at 1723 |

ATGTGGCCGAGCCTGCTGCTGCTCCTGCTGTTGCCGGGGCCCCCGCCCGTCGCCGGCTTGGAAGACGC
TGCCTTCCCCCACCTGGGGGAGAGCTTGCAGCCCCTGCCCCGGGCCTGTCCCCTGCGCTGCTCCTGCC
CCCGAGTCGACACTGTGGACTGTGATGGCTTGGACCTTCGAGTGTTCCCGGACAACATCACCAGAGCC
GCTCAGCACCTCTCCCTGCAGAACAACCAGCTCCAGGAACTCCCCTACAATGAGCTGTCCCGCCTCAG
TGGCCTGCGAACCCTCAACCTCCACAACAACCTCATCTCCTCCGAAGGTCTGCCTGACGAGGCCTTCG
AGTCCCTCACCCAGCTGCAGCACCTCTGCGTGGCTCACAACAAGCTCTCAGTGGCCCCTCAGTTTCTG
CCCCGGTCCCTCCGTGTCGCGGATCTGGCTGCCAACCAAGTGATGGAGATCTTCCCCCTCACCTTTGG
GGAGAAGCCGGCACTCAGGTCCGTGTACCTCCACAACAACCAGCTGAGCAACGCTGGCCTGCCCCCCG
ACGCCTTCCGCGGCTCCGAGGCCATCGCCACCCTCAGCCTCTCCAACAACCAGCTCAGCTACCTGCCG
CCCAGCCTGCCGCCCTCACTCGAGCGGCTCCACCTGCAGAACAATCTCATCTCCAAGGTGCCCCGAGG
AGCCCTGAGCCGCCAGACTCAACTCCGTGAGCTCTACCTCCAGCACAACCAGCTGACAGACAGTGGCC
TGGATGCCACCACCTTCAGCAAGCTGCATAGCCTTGAATACCTGGATCTCTCCCACAACCAGCTGACC
ACAGTGCCCGCCGGCCTGCCCCGGACCCTGGCTATCCTGCACCTGGGCCGCAACCGCATCCGGCAGGT
GGAGGCGGCTCGGCTGCACGGGGCGCGTGGTCTGCGCTATTTGTTGCTGCAGCACAACCAGCTGGGGA
GCTCAGGGCTGCCCGCCGGGGCTCTGCGGCCGCTGCGGGGCCTGCACACGCTGCACCTCTATGGCAAT
GGGCTGGACCGCGTGCCTCCAGCCCTGCCCCGCCGCCTGCGTGCCCTGGTGCTGCCCCACAACCACGT
GGCCGCGCTGGGTGCCCGTGACCTGGTCGCCACACCGGGCCTGACGGAGCTTAACCTGGCCTATAACC
GCCTGGCCAGCGCCCGTGTGCACCACCGGGCCTTCCGCCGGTTGCGTGCCCTGCGCAGCCTCGACCTG
GCAGGGAATCAGCTAACCCGGCTGCCCATGGGCCTGCCCACTGGCCTGCGCACCCTGCAGCTGCAACG
CAACCAGCTGCGGATGCTCGAGCCCGAGCCTCTGGCCGGCCTGGACCAACTGCGGGAGCTCAGCCTGG
CGCACAACCGGCTCCGGGTCGGCGACATCGGGCCAGGCACCTGGCATGAGCTCCAAGCCCTCCAGATG
CTGGACCTCAGCCACAATGAGCTGTCCTTTGTGCCCCCGGACCTGCCTGAGGCCCTAGAGGAGCTGCA
CCTCGAGGGCAACCGCATCGGCCACGTGGGCCCCGAGGCCTTCCTCAGCACACCCCGCCTGCGTGCCC
TCTTCCTCAGGGCCAACAGGCTTCACATGACGAGCATCGCGGCTGAGGCCTTCCTGGGGCTCCCAAAC
CTGCGTGTGGTGGACACGGCAGGGAATCCGGAGCAGGTCCTGATCCGGCTGCCTCCCACCACCCCACG
TGGGCCACGGGCAGGGGGCCCCTGATCCTAGAGAGGCCCAGCAGAGCAGCTCAGACTCCTGGGACTCC
GCTGGGCCGTGGACTGAGGAGACAACGCCCACCAGGGGCCCTTGGTCTGGCTCTCCTGGGCCTCCAGG
GCTGGGCCTGCTCTGCCTGCCACTGGCCGAGACACAGAGGCACACAGCTGGCATACTCCAGGCTCACA
GACCACGCCGGCCTGGCGGGACACACCCTACCCCAAACTCCCAACACAGATGGAGGCAGCAACAATAA
AGCCA

| | | | |
|---|---|---|---|
| NOV42d, CG56904-04 | SEQ ID NO:978 | 574 aa | MW at 63284.0 kD |
| Protein Sequence | | | |

MWPSLLLLLLLPGPPPVAGLEDAAFPHLGESLQPLPRACPLRCSCPRVDTVDCDGLDLRVFPDNITRA
AQHLSLQNNQLQELPYNELSRLSGLRTLNLHNNLISSEGLPDEAFESLTQLQHLCVAHNKLSVAPQFL
PRSLRVADTAANQVMEIFPLTFGEKPALRSVYLHNNQLSNAGLPPDAFRGSEAIATLSLSNNQLSYLP
PSLPPSLERLHLQNNLISKVPRGALSRQTQLRELYLQHNQLTDSGLDATTFSKLHSLEYLDLSNNQLT
TVPAGLPRTLAILHLGRNRIRQVEAARLHGARGLRYLLLQHNQLGSSGLPAGALRPLRGLHTLHLYGN
GLDRVPPALPRRLRALVLPHNHVAALGARDLVATPGLTELNLAYNRLASARVHHRAFRRLRALRSLDL
AGNQLTRLPMGLPTGLRTLQLQRNQLRMLEPEPLAGLDQLRELSLAHNRLRVGDIGPGTWHELQALQM

| | | |
|---|---|---|
| NOV42e, 268824694 | SEQ ID NO:979 | 1201 bp |
| DNA Sequence | ORF Start: at 2 | ORF Stop: end of sequence |

CACCGGATCCACCATGTGGCCGAGCCTGCTGCTGCTCCTGCTGTTGCCGGGGCCCCCGCCCGTCGCCG
GTTTGGAAGACGCTGCCTTCCCCCACCTGGGGGAGAGCTTGCAGCCCCTGCCCCGGGCCTGTCCCCTG
CGCTGCTCCTGCCCCCGAGTCGACACTGTGGACTGTGATGGCTTGGACCTTCGAGTGTTCCCGGACAA
CATCACCAGAGCCGCTCAGCACCTCTCCCTGCAGAACAACCAGCTCCAGGAACTCCCCTACAATGAGC
TGTCCCGCCTCAGTGGCCTGCGAACCCTCAACCTCCACAACAACCTCATCTCCTCCGAAGGCCTGCCT
GACGAGGCCTTCGAGTCCCTCACCCAGCTGCAGCACCTCTGCGTGGCTCACAACAAGCTCTCAGTGGC
CCCTCAGTTTCTGCCCCGGTCCCTCCGTGTCGCGGATCTGGCTGCCAACCAAGTGATGGAGATCTTCC
CCCTCACCTTTGGGGAGAAGCCGGCACTCAGGTCCGTGTACCTCCACAACAACCAGCTGAGCAACGCT
GGCCTGCCCCCCGACGCCTTCCGCGGCTCCGAGGCCATCGCCACCCTCAGCCTCTCCAACAACCAGCT
CAGCTACCTGCCGCCCAGCCTGCCGCCCTCACTCGAGCGGCTCCACCTGCAGAACAATCTCATCTCCA
AGGTGCCCCGAGGAGCCCTGAGCCGCCAGACTCAACTCCGTGAGCTCTACCTCCAGCACAACCAGCTG
ACAGACAGTGGCCTGGATGCCACCACCTTCAGCAAGCTGCATAGCCTTGAATACCTGGATCTCTCCCA
GCATCCGGCAGATGCTGGACCTCAGCCACAATGAGCTGTCCTTTGTGCCCCCGGACCTGCCTGAGGCC
CTAGAGGAGCTGCACCTCGAGGGCAACCGCATCGGCCACGTGGGCCCCGAGGCCTTCCTCAGCACACC
CCGCCTGCGTGCCCTCTTCCTCAGGGCCAACAGGCTTCACATGACGAGCATCGCGGCTGAGGCCTTCC
TGGGGCTCCCAAACCTGCGTGTGGTGGACACGGCAGGGAATCCGGAGCAGGTCCTGATCCGGCTGCCT
CCCACCACCCCACGTGGGCCACGGGCAGGGGGCCCCAAGCTTGGC

| | | | |
|---|---|---|---|
| NOV42e, 268824694 | SEQ ID NO:980 | 400 aa | MW at 43934.7 kD |
| Protein Sequence | | | |

TGSTMWPSLLLLLLLPGPPPVAGLEDAAFPHLGESLQPLPPACPLRCSCPRVDTVDCDGLDLRVFPDN
ITRAAQHLSLQNNQLQELPYNELSRLSGLRTLNLHNNLISSEGLPDEAFESLTQLQHLCVAHNKLSVA
PQFLPRSLRVADLAANQVMEIFPLTFGEKPALRSVYLHNNQLSNAGLPPDAFRGSEAIATLSLSNNQL
SYLPPSLPPSLERLHLQNNLISKVPRGALSRQTQLRELYLQHNQLTDSGLDATTFSKLHSLEYLDLSH
NQLTTVPAGLPRTLAILHLGRNRIRQMLDLSHNELSFVPPDLPEALEELHLEGNRIGHVGPEAFLSTP
RLRALFLRANRLHMTSIAAEAFLGLPNLRVVDTAGNPEQVLIRLPPTTPRGPRAGGPKLG

| | | |
|---|---|---|
| NOV42f, 283146542 | SEQ ID NO:981 | 1744 bp |
| DNA Sequence | ORF Start: at 2 | ORF Stop: end of sequence |

CACCGGATCCACCATGTGGCCGAGCCTGCTGCTGCTCCTGCTGTTGCCGGGGCCCCCGCCCGTCGCCG
GCTTGGAAGACGCTGCCTTCCCCCACCTGGGGGAGAGCTTGCAGCCCCTGCCCCGGGCCTGTCCCCTG
CGCTGCTCCTGCCCCCGAGTCGACACTGTGGACTGTGATGGCTTGGACCTTCGAGTGTTCCCGGACAA
CATCACCAGAGCCGCTCAGCACCTCTCCCTGCAGAACAACCAGCTCCAGGAACTCCCCTACAATGAGC
TGTCCCGCCTCAGTGGCCTGCGAACCCTCAACCTCCACAACAACCTCATCTCCTCCGAAGGCCTGCCT
GACGAGGCCTTCGAGTCCCTCACCCAGCTGCAGCACCTCTGCGTGGCTCACAACAAGCTCTCAGTGGC
CCCTCAGTTTCTGCCCCGGTCCCTCCGTGTCGCGGATCTGGCTGCCAACCAAGTGATGGAGATCTTCC
CCCTCACCTTTGGGGAGAAGCCGGCACTCAGGTCCGTGTACCTCCACAACAACCAGCTGAGCAACGCT
GGCCTGCCCCCCGACGCCTTCCGCGGCTCCGAGGCCATCGCCACCCTCAGCCTCTCCAACAACCAGCT
CAGCTACCTGCCGCCCAGCCTGCCGCCCTCACTCGAGCGGCTCCACCTGCAGAACAATCTCATCTCCA
AGGTGCCCCGAGGAGCCCTGAGCCGCCAGACTCAACTCCGTGAGCTCTACCTCCAGCACAACCAGCTG
ACAGACAGTGGCCTGGATGCCACCACCTTCAGCAAGCTGCATAGCCTTGAATACCTGGATCTCTCCCA
CAACCAGCTGACCACAGTGCCCGCCGGCCTGCCCCGGACCCTGGCTATCCTGCACCTGGGCCGCAACC
GCATCCGGCAGGTGGAGGCGGCTCGGCTGCACGGGGCGCGTGGTCTGCGCTATTTGTTGCTGCAGCAC
AACCAGCTGGGGAGCTCAGGGCTGCCCGCCGGGGCTCTGCGGCCGCTGCGGGGCCTGCACACGCTGCA
CCTCTATGGCAATGGGCTGGACCGCGTGCCTCCAGCCCTGCCCCGCCGCCTGCGTGCCCTGGTGCTGC
CCCACAACCACGTGGCCGCGCTGGGTGCCCGTGACCTGGTCGCCACACCGGGCCTGACGGAGCTTAAC
CTGGCCTATAACCGCCTGGCCAGCGCCCGTGTGCACCACCGGGCCTTCCGCCGGTTGCGTGCCCTGCG
CAGCCTCGACCTGGCAGGGAATCAGCTAACCCGGCTGCCCATGGGCCTGCCCACTGGCCTGCGCACCC
TGCAGCTGCAACGCAACCAGCTGCGGATGCTCGAGCCCGAGCCTCTGGCCGGCCTGGACCAACTGCGG
GAGCTCAGCCTGGCGCACAACCGGCTCCGGGTCGGCGACATCGGGCCAGGCACCTGGCATGAGCTCCA
AGCCCTCCAGATGCTGGACCTCAGCCACAATGAGCTGTCCTTTGTGCCCCCGGACCTGCCTGAGGCCC
TGGAGGAGCTGCACCTCGAGGGCAACCGCATCGGCCACGTGGGCCCCGAGGCCTTCCTCAGCACACCC
CGCCTGCGTGCCCTCTTCCTCAGGGCCAACAGGCTTCACATGACGAGCATCGCGGCTGAGGCCTTCCT
GGGGCTCCCAAACCTGCGTGTGGTGGACACGGCAGGGAATCCGGAGCAGGTCCTGATCCGGCTGCCTC
CCACCACCCCACGTGGGCCACGGGCAGGGGGCCCCAAGCTTGGC

| | | | |
|---|---|---|---|
| NOV42f, 283146542 | SEQ ID NO:982 | 581 aa | MW at 63928.7 kD |
| Protein Sequence | | | |

TGSTMWPSLLLLLLLPGPPPVAGLEDAAFPHLGESLQPLPRACPLRCSCPRVDTVDCDGLDLRVFPDN
ITRAAQHLSLQNNQLQELPYNELSRLSGLRTLNLHNNLISSEGLPDEAFESLTQLQHLCVAHNKLSVA
PQFLPRSLRVADLAANQVMEIFPLTFGEKPALRSVYLHNNQLSNAGLPPDAFRGSEAIATLSLSNNQL
SYLPPSLPPSLERLHLQNNLISKVPRGALSRQTQLRELYLQHNQLTDSGLDATTFSKLHSLEYLDLSH
NQLTTVPAGLPRTLAILHLGRNRIRQVEAARLHGARGLRYLLLQHNQLGSSGLPAGALRPLRGLHTLH
LYGNGLDRVPPALPRRLRALVLPHNHVAALGARDLVATPGLTELNLAYNRLASARVHHEAFRRLRALR
SLDLAGNQLTRLPMGLPTGLRTLQLQRNQLRMLEPEPLAGLDQLRELSLAHNRLRVGDIGPGTWHELQ
ALQMLDLSHNELSFVPPDLPEALEELHLEGNRIGHVGPEAFLSTPRLRALFLRANRLHMTSIAAEAFL
IGLPNLRVVDTAGNPEQVLIRLPPTTPRGPRAGGPKLG

| | | |
|---|---|---|
| NOV42g, 308521214 | SEQ ID NO:983 | 1744 bp |
| DNA Sequence | ORF Start: at 2 | ORF Stop: end of sequence |

CACCGGATCCACCATGTGGCCGAGCCTGCTGCTGCTCCTGCTGTTGCCGGGGCCCCCGCCCGTCGCCG
GCTTGGAAGACGCTGCCTTCCCCCACCTGGGGGAGAGCTTGCAGCCCCTGCCCCGGGCCTGTCCCCTG
CGCTGCTCCTGCCCCCGAGTCGACACTGTGGACTGTGATGGCTTGGACCTTCGAGTGTTCCCGGACAA
CATCACCAGAGCCGCTCAGCACCTCTCCCTGCAGAACAACCAGCTCCAGGAACTCCCCTACAATGAGC
TGTCCCGCCTCAGTGGCCTGCGAACCCTCAACCTCCACAACAACCTCATCTCCTCCGAAGGCCTGCCT
GACGAGGCCTTCGAGTCCCTCACCCAGCTGCAGCACCTCTGCGTGGCTCACAACAAGCTCTCAGTGGC
CCCTCAGTTTCTGCCCCGGTCCCTCCGTGTCGCGGATCTGGCTGCCAACCAAGTGATGGAGATCTTCC
CCCTCACCTTTGGGGAGAAGCCGGCACTCAGGTCCGTGTACCTCCACAACAACCAGCTGAGCAACGCT
GGCCTGCCCCCCGACGCCTTCCGCGGCTCCGAGGCCATCGCCACCCTCAGCCTCTCCAACAACCAGCT
CAGCTACCTGCCGCCCAGCCTGCCGCCCTCACTCGAGCGGCTCCACCTGCAGAACAATCTCATCTCCA
AGGTGCCCCGAGGAGCCCTGAGCCGCCAGACTCAACTCCGTGAGCTCTACCTCCAGCACAACCAGCTG
CAACCAGCTGACCACAGTGCCCGCCGGCCTGCCCCGGACCCTGGCTATCCTGCACCTGGGCCGCAACC
GCATCCGGCAGGTGGAGGCGGCTCGGCTGCACGGGGCGCGTGGTCTGCGCTATTTGTTGCTGCAGCAC
AACCAGCTGGGGAGCTCAGGGCTGCCCGCCGGGGCTCTGCGGCCGCTGCGGGGCCTGCACACGCTGCA
CCTCTATGGCAATGGGCTGGACCGCGTGCCTCCAGCCCTGCCCCGCCGCCTGCGTGCCCTGGTGCTGC
CCCACAACCACGTGGCCGCGCTGGGTGCCCGTGACCTGGTCGCCACACCGGGCCTGACGGAGCTTAAC
CTGGCCTATAACCGCCTGGCCAGCGCCCGTGTGCACCACCGGGCCTTCCGCCGGTTGCGTGCCCTGCG
CAGCCTCGACCTGGCAGGGAATCAGCTAACCCGGCTGCCCATGGGCCTGCCCACTGGCCTGCGCACCC
TGCAGCTGCAACGCAACCAGCTGCGGATGCTCGAGCCCGAGCCTCTGGCCGGCCTGGACCAACTGCGG
GAGCTCAGCCTGGCGCACAACCGGCTCCGGGTCGGCGACATCGGGCCAGGCACCTGGCATGAGCTCCA
AGCCCTCCAGATGCTGGACCTCAGCCACAATGAGCTGTCCTTTGTGCCCCCGGACCTGCCTGAGGCCC
TGGAGGAGCTGCACCTCGAGGGCAACCGCATCGGCCACGTGGGCCCCGAGGCCTTCCTCAGCACACCC
CGCCTGCGTGCCCTCTTCCTCAGGGCCAACAGGCTTCACATGACGAGCATCGCGGCTGAGGCCTTCCT
GGGGCTCCCAAACCTGCGTGTGGTGGACACGGCAGGGAATCCGGAGCAGGTCCTGATCCGGCTGCCTC
CCACCACCCCACGTGGGCCACGGGCAGGGGGCCCCGAATTCGGC

| | | | |
|---|---|---|---|
| NOV42g, 308521214 | SEQ ID NO:984 | 581 aa | MW at 63963.7 kD |
| Protein Sequence | | | |

TGSTMWPSLLLLLLLPGPPPVAGLEDAAFPHLGESLQPLPRACPLRCSCPRVDTVDCDGLDLRVFPDN
ITRAAQHLSLQNNQLQELPYNELSRLSGLRTLNLHNNLISSEGLPDEAFESLTQLQHLCVAHNKLSVA
PQFLPRSLRVADLAANQVMEIFPLTFGEKPALRSVYLHNNQLSNAGLPPDAFRGSEAIATLSLSNNQL
SYLPPSLPPSLERLHLQNNLISKVPRGALSRQTQLRELYLQHNQLTDSGLDATTFSKLHSLEYLDLSH
NQLTTVPAGLPRTLAILHLGRNRIRQVEAARLHGARGLRYLLLQHNQLGSSGLPAGALRPLRGLHTLH
LYGNGLDRVPPALPRRLRALVLPHNHVAALGARDLVATPGLTELNLAYNRLASARVHHRAFRRLRALR
SLDLAGNQLTRLPMGLPTGLRTLQLQPNQLRMLEPEPLAGLDQLRELSLAHNRLRVGDIGPGTWHELQ
ALQMLDLSHNELSFVPPDLPEALEELHLEGNRIGHVGPEAFLSTPRLRALFLRANRLHMTSIAAEAFL
GLPNLRVVDTAGNPEQVLIRLPPTTPRGPRAGGPEFG

| | | |
|---|---|---|
| NOV42h, CG56904-02 | SEQ ID NO:985 | 1506 bp |
| DNA Sequence | ORF Start: ATG at 1 | ORF Stop: TGA at 1504 |

ATGATGAACAACCAGCTCCAGGAACTCCCCTACAATGAGCTGTCCCGCCTCAGTGGCCTGCGAACCCT
CAACCTCCACAACAACCTCATCTCCTCCGAAGGCCTGCCTGACGAGGCCTTCGAGTCCCTCACCCAGC
TGCAGCACCTCTGCGTGGCTCACAACAAGCTCTCAGTGGCCCCTCAGTTTCTGCCCCGGTCCCTCCGT
GTCGCGGATCTGGCTGCCAACCAAGTGATGGAGATCTTCCCCCTCACCTTTGGGGAGAAGCCGGCACT
CAGGTCCGTGTACCTCCACAACAACCAGCTGAGCAACGCTGGCCTGCCCCCCGACGCCTTCCGCGGCT
CCGAGGCCATCGCCACCCTCAGCCTCTCCAACAACCAGCTCAGCTACCTGCCGCCCAGCCTGCCGCCC
TCACTCGAGCGGCTCCACCTGCAGAACAATCTCATCTCCAAGGTGCCCCGAGGAGCCCTGAGCCGCCA
GACTCAACTCCGTGAGCTCTACCTCCAGCACAACCAGCTGACAGACAGTGGCCTGGATGCCACCACCT
CTGCCCCGGACCCTGGCTATCCTGCACCTGGGCCGCAACCGCATCCGGCAGGTGGAGGCGGCTCGGCT
GCACGGGGCGCGTGGTCTGCGCTATTTGTTGCTGCAGCACAACCAGCTGGGGAGCTCAGGGCTGCCCG
CCGGGGCTCTGCGGCCGCTGCGGGGCCTGCACACGCTGCACCTCTATGGCAATGGGCTGGACCGCGTG
CCTCCAGCCCTGCCCCGCCGCCTGCGTGCCCTGGTGCTGCCCCACAACCACGTGGCCGCGCTGGGTGC
CCGTGACCTGGTCGCCACACCGGGCCTGACGGAGCTTAACCTGGCCTATAACCGCCTGGCCAGCGCCC
GTGTGCACCACCGGGCCTTCCGCCGGTTGCGTGCCCTGCGCAGCCTCGACCTGGCAGGGAATCAGCTA
ACCCGGCTGCCCATGGGCCTGCCCACTGGCCTGCGCACCCTGCAGCTGCAACGCAACCAGCTGCGGAT
GCTCGAGCCCGAGCCTCTGGCCGGCCTGGACCAACTGCGGGAGCTCAGCCTGGCGCACAACCGGCTCC
GGGTCGGCGACATCGGGCCAGGCACCTGGCATGAGCTCCAAGCCCTCCAGATGCTGGACCTCAGCCAC
AATGAGCTGTCCTTTGTGCCCCCGGACCTGCCTGAGGCCCTAGAGGAGCTGCACCTCGAGGGCAACCG
CATCGGCCACGTGGGCCCCGAGGCCTTCCTCAGCACACCCCGCCTGCGTGCCCTCTTCCTCAGGGCCA
ACAGGCTTCACATGACGAGCATCGCGGCTGAGGCCTTCCTGGGGCTCCCAAACCTGCGTGTGGTGGAC
GGGCCCCTGA

| | | | |
|---|---|---|---|
| NOV42h, CG56904-02 | SEQ ID NO:986 | 501 aa | MW at 55426.1 kD |
| Protein Sequence | | | |

MMNNQLQELPYNELSRLSGLRTLNLHNNLISSEGLPDEAFESLTQLQHLCVAHNKLSVAPQFLPRSLR
VADLAANQVMEIFPLTFGEKPALRSVYLHNNQLSNAGLPPDAFRGSEAIATLSLSNNQLSYLPPSLPP
SLERLHLQNNLISKVPRGALSRQTQLRELYLQHNQLTDSGLDATTFSKLHSLEYLDLSHNQLTTVPAG
LPRTLAILHLGRNRIRQVEAARLHGARGLRYLLLQHNQLGSSGLPAGALRPLRGLHTLHLYGNGLDRV
PPALPRRLRALVLPHNHVAALGARDLVATPGLTELNLAYNRLASARVHHRAFRRLRALRSLDLAGNQL
TRLPMGLPTGLRTLQLQRNQLRMLEPEPLAGLDQLRELSLAHNRLRVGDIGPGTWHELQALQMLDLSH
NELSFVPPDLPEALEELHLEGNRIGHVGPEAFLSTPRLRALFLRANRLHMTSIAAEAFLGLPNLRVVD
TAGNPEQVLIRLPPTTPRGPRAGGP

| | | |
|---|---|---|
| NOV42i, CG56904-03 | SEQ ID NO:987 | 1239 bp |
| DNA Sequence | ORF Start: ATG at 1 | ORF Stop: TGA at 1237 |

ATGATGAACAACCAGCTCCAGGAACTCCCCTACAATGAGCTGTCCCGCCTCAGTGGCCTGCGAACCCT
CAACCTCCACAACAACCTCATCTCCTCCGAAGGCCTGCCTGACGAGGCCTTCGAGTCCCTCACCCAGC
TGCAGCACCTCTGCGTGGCTCACAACAAGAACAATCTCATCTCCAAGGTGCCCCGAGGAGCCCTGAGC
CGCCAGACTCAACTCCGTGAGCTCTACCTCCAGCACAACCAGCTGACAGACAGTGGCCTGGATGCCAC
CACCTTCAGCAAGCTGCATAGCCTTGAATACCTGGATCTCTCCCACAACCAGCTGACCACAGTGCCCG
CCGGCCTGCCCCGGACCCTGGCTATCCTGCACCTGGGCCGCAACCGCATCCGGCAGGTGGAGGCGGCT
CGGCTGCACGGGGCGCGTGGTCTGCGCTATTTGTTGCTGCAGCACAACCAGCTGGGGAGCTCAGGGCT
GCCCGCCGGGGCTCTGCGGCCGCTGCGGGGCCTGCACACGCTGCACCTCTATGGCAATGGGCTGGACC
GCGTGCCTCCAGCCCTGCCCCGCCGCCTGCGTGCCCTGGTGCTGCCCCACAACCACGTGGCCGCGCTG
GGTGCCCGTGACCTGGTCGCCACACCGGGCCTGACGGAGCTTAACCTGGCCTATAACCGCCTGGCCAG
CGCCCGTGTGCACCACCGGGCCTTCCGCCGGTTGCGTGCCCTGCGCAGCCTCGACCTGGCAGGGAATC
AGCTAACCCGGCTGCCCATGGGCCTGCCCACTGGCCTGCGCACCCTGCAGCTGCAACGCAACCAGCTG
CGGATGCTCGAGCCCGAGCCTCTGGCCGGCCTGGACCAACTGCGGGAGCTCAGCCTGGCGCACAACCG
GCTCCGGGTCGGCGACATCGGGCCAGGCACCTGGCATGAGCTCCAAGCCCTCCAGATGCTGGACCTCA
GCCACAATGAGCTGTCCTTTGTGCCCCCGGACCTGCCTGAGGCCCTAGAGGAGCTGCACCTCGAGGGC
AACCGCATCGGCCACGTGGGCCCCGAGGCCTTCCTCAGCACACCCCGCCTGCGTGCCCTCTTCCTCAG
GGCCAACAGGCTTCACATGACGAGCATCGCGGCTGAGGCCTTCCTGGGGCTCCCAAACCTGCGTGTGG
TGGACACGGCAGGGAATCCGGAGCAGGTCCTGATCCGGCTGCCTCCCACCACCCCACGTGGGCCACGG
GCAGGGGGCCCCTGA

| | | | |
|---|---|---|---|
| NOV42i, CG56904-03 | SEQ ID NO:988 | 412 aa | MW at 45723.1 kD |
| Protein Sequence | | | |

MMNNQLQELPYNELSRLSGLRTLNLHNNLISSEGLPDEAFESLTQLQHLCVAHNKNNLISKVPRGALS
RQTQLRELYLQHNQLTDSGLDATTFSKLHSLEYLDLSHNQLTTVPAGLPRTLAILHLGRNRIRQVEAA
RLHGARGLRYLLLQHNQLGSSGLPAGALRPLRGLHTLHLYGNGLDRVPPALPRRLRALVLPHNHVAAL
GAPDLVATPGLTELNLAYNRLASARVHHRAFRRLRALRSLDLAGNQLTRLPMGLPTGLRTLQLQRNQL
RMLEPEPLAGLDQLRELSLAHKRLRVGDIGPGTWHELQALQMLDLSHNELSFVPPDLPEALEELHLEG
NRIGHVGPEAFLSTPRLRALFLRANRLHMTSIAAEAFLGLPNLRVVDTAGNPEQVLIRLPPTTPRGPR
AGGP

| | | |
|---|---|---|
| NOV42 , CG56904-05 | SEQ ID NO:989 | 1710 bp |
| DNA Sequence | ORF Start: ATG at 1 | ORF Stop: TGA at 1456 |

ATGTGGCCGAGCCTGCTGCTGCTCCTGCTGTTGCCGGGGCCCCCGCCCGTCGCCGGCTTGGAAGACGC
TGCCTTCCCCCACCTGGGGGAGAGCTTGCAGCCCCTGCCCCGGGCCTGTCCCCTGCGCTGCTCCTGCC
CCCGAGTCGACACTGTGGACTGTGATGGCTTGGACCTTCGAGTGTTCCCGGACAACATCACCAGAGCC
GCTCAGCACCTCTCCCTGCAGAACAACCAGCTCCAGGAACTCCCCTACAATGAGCTGTCCCGCCTCAG
TGGCCTGCGAACCCTCAACCTCCACAACAACCTCATCTCCTCCGAAGGTCTGCCTGACGAGGCCTTCG
AGTCCCTCACCCAGCTGCAGCACCTCTGCGTGGCTCACAACAAGAACAATCTCATCTCCAAGGTGCCC
CGAGGAGCCCTGAGCCGCCAGACTCAACTCCGTGAGCTCTACCTCCAGCACAACCAGCTGACAGACAG
TGGCCTGGATGCCACCACCTTCAGCAAGCTGCATAGCCTTGAATACCTGGATCTCTCCCACAACCAGC
TGACCACAGTGCCCGCCGGCCTGCCCCGGACCCTGGCTATCCTGCACCTGGGCCGCAACCGCATCCGG
CAGGTGGAGGCGGCTCGGCTGCACGGGGCGCGTGGTCTGCGCTATTTGTTGCTGCAGCACAACCAGCT
GGGGAGCTCAGGGCTGCCCGCCGGGGCTCTGCGGCCGCTGCGGGGCCTGCACACGCTGCACCTCTATG
GCAATGGGCTGGACCGCGTGCCTCCAGCCCTGCCCCGCCGCCTGCGTGCCCTGGTGCTGCCCCACAAC
CACGTGGCCGCGCTGGGTGCCCGTGACCTGGTCGCCACACCGGGCCTGACGGAGCTTAACCTGGCCTA
TAACCGCCTGGCCAGCGCCCGTGTGCACCACCGGGCCTTCCGCCGGTTGCGTGCCCTGCGCAGCCTCG
ACCTGGCAGGGAATCAGCTAACCCGGCTGCCCATGGGCCTGCCCACTGGCCTGCGCACCCTGCAGCTG
CAACGCAACCAGCTGCGGATGCTCGAGCCCGAGCCTCTGGCCGGCCTGGACCAACTGCGGGAGCTCAG
CCTGGCGCACAACCGGCTCCGGGTCGGCGACATCGGGCCAGGCACCTGGCATGAGCTCCAAGCCCTCC
AGATGCTGGACCTCAGCCACAATGAGCTGTCCTTTGTGCCCCCGGACCTGCCTGAGGCCCTAGAGGAG
CTGCACCTCGAGGGCAACCGCATCGGCCACGTGGGCCCCGAGGCCTTCCTCAGCACACCCCGCCTGCG
TGCCCTCTTCCTCAGGGCCAACAGGCTTCACATGACGAGCATCGCGGCTGAGGCCTTCCTGGGGCTCC
CAAACCTGCGTGTGGTGGACACGGCAGGGAATCCGGAGCAGGTCCTGATCCGGCTGCCTCCCACCACC
CCACGTGGGCCACGGGCAGGGGGCCCCTGATCCTAGAGAGGCCCAGCAGAGCAGCTCAGACTCCTGGG
ACTCCGCTGGGCCGTGGACTGAGGAGACAACGCCCACCAGGGGCCCTTGGTCTGGCTCTCCTGGGCCT
CCAGGGCTGGGCCTGCTCTGCCTGCCACTGGCCGAGACACAGAGGCACACAGCTGGCATACTCCAGGC
TCACAGACCACGCCGGCCTGGCGGGACACACCCTACCCCAAACTCCCAACACAGATGGAGGCAGCAAC
AATAAAGCCA

| | | | |
|---|---|---|---|
| NOV42 , CG56904-05 | SEQ ID NO:990 | 485aa | MW at 53581.10 kD |
| Protein Sequence | | | |

MWPSLLLLLLLPGPPPVAGLEDAAFPHLGESLQPLPRACPLRCSCPRVDTVDCDGLDLRVFPDNITRA
AQHLSLQNNQLQELPYNELSRLSGLRTLNLHNNLISSEGLPDEAFESLTQLQHLCVAHNKNNLISKVP
RGALSRQTQLRELYLQHNQLTDSGLDATTFSKLHSLEYLDLSHNQLTTVPAGLPRTLAILHLGRNRIR
QVEAARLHGARGLRYLLLQHNQLGSSGLPAGALRPLRGLHTLHLYGNGLDRVPPALPRRLRALVLPHN
HVAALGARDLVATPGLTELNLAYNRLASARVHHRAFRRLRALRSLDLAGNQLTRLPMGLPTGLRTLQL
QRNQLRMLEPEPLAGLDQLRELSLAHNRLRVGDIGPGTWJELQALQMLDLSHNELSFVPPDLPEALEE
LHLEGNRIGHVGPEAFLSTPRLRALFLRANRLHMTSIAAEAFLGLPNLRVVDTAGNPEQVLIRLPPTT
PRGPRAGGP

| | | |
|---|---|---|
| NOV42k, CG56904-06 | SEQ ID NO:991 | 1992 bp |
| DNA Sequence | ORF Start: ATG at 1 | ORF Stop: TGA at 1738 |

ATGGGGAGGCCCACCCAGTGGCCGAGCCTGCTGCTGCTCCTGCTGTTGCCGGGGCCCCCGCCCGTCGC
CGGCTTGGAAGACGCTGCCTTCCCCCACCTGGGGGAGAGCTTGCAGCCCCTGCCCCGGGCCTGTCCCC
TGCGCTGCTCCTGCCCCCGAGTCGACACTGTGGACTGTGATGGCTTGGACCTTCGAGTGTTCCCGGAC
AACATCACCAGAGCCGCTCAGCACCTCTCCCTGCAGAACAACCAGCTCCAGGAACTCCCCTACAATGA
GCTGTCCCGCCTCAGTGGCCTGCGAACCCTCAACCTCCACAACAACCTCATCTCCTCCGAAGGTCTGC
CTGACGAGGCCTTCGAGTCCCTCACCCAGCTGCAGCACCTCTGCGTGGCTCACAACAAGCTCTCAGTG
GCCCCTCAGTTTCTGCCCCGGTCCCTCCGTGTCGCGGATCTGGCTGCCAACCAAGTGATGGAGATCTT
CCCCCTCACCTTTGGGGAGAAGCCGGCACTCAGGTCCGTGTACCTCCACAACAACCAGCTGAGCAACG
CTGGCCTGCCCCCCGACGCCTTCCGCGGCTCCGAGGCCATCGCCACCCTCAGCCTCTCCAACAACCAG
CTCAGCTACCTGCCGCCCAGCCTGCCGCCCTCACTCGAGCGGCTCCACCTGCAGAACAATCTCATCTC
CAAGGTGCCCCGAGGAGCCCTGAGCCGCCAGACTCAACTCCGTGAGCTCTACCTCCAGCACAACCAGC
TGACAGACAGTGGCCTGGATGCCACCACCTTCAGCAAGCTGCATAGCCTTGAATACCTGGATCTCTCC
CACAACCAGCTGACCACAGTGCCCGCCGGCCTGCCCCGGACCCTGGCTATCCTGCACCTGGGCCGCAA
CCGCATCCGGCAGGTGGAGGCGGCTCGGCTGCACGGGGCGCGTGGTCTGCGCTATTTGTTGCTGCAGC
ACAACCAGCTGGGGAGCTCAGGGCTGCCCGCCGGGGCTCTGCGGCCGCTGCGGGGCCTGCACACGCTG
CACCTCTATGGCAATGGGCTGGACCGCGTGCCTCCAGCCCTGCCCCGCCGCCTGCGTGCCCTGGTGCT
GCCCCACAACCACGTGGCCGCGCTGGGTGCCCGTGACCTGGTCGCCACACCGGGCCTGACGGAGCTTA
ACCTGGCCTATAACCGCCTGGCCAGCGCCCGTGTGCACCACCGGGCCTTCCGCCGGTTGCGTGCCCTG
CGCAGCCTCGACCTGGCAGGGAATCAGCTAACCCGGCTGCCCATGGGCCTGCCCACTGGCCTGCGCAC
CCTGCAGCTGCAACGCAACCAGCTGCGGATGCTCGAGCCCGAGCCTCTGGCCGGCCTGGACCAACTGC
GGGAGCTCAGCCTGGCGCACAACCGGCTCCGGGTCGGCGACATCGGGCCAGGCACCTGGCATGAGCTC
CAAGCCCTCCAGATGCTGGACCTCAGCCACAATGAGCTGTCCTTTGTGCCCCCGGACCTGCCTGAGGC
CCTAGAGGAGCTGCACCTCGAGGGCAACCGCATCGGCCACGTGGGCCCCGAGGCCTTCCTCAGCACAC
CCCGCCTGCGTGCCCTCTTCCTCAGGGCCAACAGGCTTCACATGACGAGCATCGCGGCTGAGGCCTTC
CTGGGGCTCCCAAACCTGCGTGTGGTGGACACGGCAGGGAATCCGGAGCAGGTCCTGATCCGGCTGCC
TCCCACCACCCCACGTGGGCCACGGGCAGGGGGCCCCTGATCCTAGAGAGGCCCAGCAGAGCAGCTCA
GACTCCTGGGACTCCGCTGGGCCGTGGACTGAGGAGACAACGCCCACCAGGGGCCCTTGGTCTGGCTC
TCCTGGGCCTCCAGGGCTGGGCCTGCTCTGCCTGCCACTGGCCGAGACACAGAGGCACACAGCTGGCA
TACTCCAGGCTCACAGACCACGCCGGCCTGGCGGGACACACCCTACCCCAAACTCCCAACACAGATGC
AGGCAGCAACAATAAAGCCA

| | | | |
|---|---|---|---|
| NOV42k, CG56904-06 | SEQ ID NO:992 | 579 aa | MW at 63823.6 kD |
| Protein Sequence | | | |

MGRPTQWPSLLLLLLLPGPPPVAGLEDAAFPHLGESLQPLPPACPLRCSCPRVDTVDCDGLDLRVFPD
NITRAAQHLSLQNNQLQELPYNELSRLSGLRTLNLHNNLISSEGLPDEAFESLTQLQHLCVAHNKLSV
APQFLPRSLRVADLAANQVMEIFPLTFGEKPALRSVYLHNNQLSNAGLPPDAFRGSEAIATLSLSNNQ
LSYLPPSLPPSLERLHLQNNLISKVPRGALSRQTQLRELYLQHNQLTDSGLDATTFSKLHSLEYLDLS
HNQLTTVPAGLPRTLAILHLGRNRIRQVEAARLHGARGLRYLLLQHNQLGSSGLPAGALRPLRGLHTL
HLYGNGLDRVPPALPRRLRALVLPHNHVAALGARDLVATPGLTELNLAYNRLASARVHHRAFRRLRAL
RSLDLAGNQLTRLPMGLPTGLRTLQLQRNQLRMLEPEPLAGLDQLRELSLAHNRLRVGDIGPGTWHEL
QALQMLDLSHNELSFVPPDLPEALEELHLEGNRIGHVGPEAFLSTPRLRALFLRANRLHMTSIAAEAF
LGLPNLRVVDTAGNPEQVLIRLPPTTPRGPRAGGP

| | | |
|---|---|---|
| NOV42l, CG56904-07 | SEQ ID NO:993 | 1278 bp |
| DNA Sequence | ORF Start: ATG at 10 | ORF Stop: at 1273 |

GGATCCACCATGGGGAGGCCCACCCAGTGGCCGAGCCTGCTGCTGCTCCTGCTGTTGCCGGGGCCCCC
GCCCGTCGCCGGCTTGGAAGACGCTGCCTTCCCCCACCTGGGGGAGAGCTTGCAGCCCCTGCCCCGGG
CCTGTCCCCTGCGCTGCTCCTGCCCCCGAGTCGACACTGTGGACTGTGATGGCTTGGACCTTCGAGTG
TTCCCGGACAACATCACCAGAGCCGCTCAGCACCTCTCCCTGCAGAACAACCAGCTCCAGGAACTCCC
CTACAATGAGCTGTCCCGCCTCAGTGGCCTGCGAACCCTCAACCTCCACAACAACCTCATCTCCTCCG
AAGGCCTGCCTGACGAGGCCTTCGAGTCCCTCACCCAGCTGCAGCACCTCTGCGTGGCTCACAACAAG
AACAATCTCATCTCCAAGGTGCCCCGAGGAGCCCTGAGCCGCCAGACTCAACTCCGTGAGCTCTACCT
CCAGCACAACCAGCTGACAGACAGTGGCCTGGATGCCACCACCTTCAGCAAGCTGCATAGCCTTGAAT
CACCTGGGCCGCAACCGCATCCGGCAGGTGGAGGCGGCTCGGCTGCACGGGGCGCGTGGTCTGCGCTA
TTTGTTGCTGCAGCACAACCAGCTGGGGAGCTCAGGGCTGCCCGCCGGGGCTCTGCGGCCGCTGCGGG
GCCTGCACACGCTGCACCTCTATGGCAATGGGCTGGACCGCGTGCCTCCAGCCCTGCCCCGCCGCCTG
CGTGCCCTGGTGCTGCCCCACAACCACGTGGCCGCGCTGGGTGCCCGTGACCTGGTCGCCACACCGGG
CCTGACGGAGCTTAACCTGGCCTATAACCGCCTGGCCAGCGCCCGTGTGCACCACCGGGCCTTCCGCC
GGTTGCGTGCCCTGCGCAGCCTCGACCTGGCAGGGAATCAGCTAACCCGGCTGCCCATGGGCCTGCCC
ACTGGCCTGCGCACCCTGCAGCTGCAACGCAACCAGCTGCGGATGCTCGAGCCCGAGCCTCTGGCCGG
CCTGGACCAACTGCGGGAGCTCAGCCTGGCGCACAACCGGCTCCGGGTCGGCGACATCGGGCCAGGCA
CCTGGCATGAGCTCCAAGCCCTCCAGGTCAGGCACAGGCTGGTTAGCCACACTGTCCCCAGGGCCCCT
CCATCCCCCTGCCTGCCCTGCCACGTCCCAAACATTCTAGTTAGCTGGGAATTC

| | | | |
|---|---|---|---|
| NOV421, CG56904-07 | SEQ ID NO:994 | 421 aa | MW at 46715.4 kD |
| Protein Sequence | | | |

MGRPTQWPSLLLLLLLPGPPPVAGLEDAAFPHLGESLQPLPRACPLRCSCPRVDTVDCDGLDLRVFPD
NITRAAQHLSLQNNQLQELPYNELSRLSGLRTLNLHNNLISSEGLPDEAFESLTQLQHLCVAHNKNNL
ISKVPRGALSRQTQLRELYLQHNQLTDSGLDATTFSKLHSLEYLDLSHNQLTTVPAGLPRTLAILHLG
RNRIRQVEAARLHGARGLRYLLLQHNQLGSSGLPAGALRPLRGLHTLHLYGNGLDRVPPALPRRLRAL
VLPHNHVAALGARDLVATPGLTELNLAYNRLASARVHHRAFRRLRALRSLDLAGNQLTRLPMGLPTGL
RTLQLQRNQLRMLEPEPLAGLDQLRELSLAHNRLRVGDIGPGTWHELQALQVRHRLVSHTVPRAPPSP
CLPCHVPNILVSW

| | | |
|---|---|---|
| NOV42m, CG56904-08 | SEQ ID NO:995 | 1744 bp |
| DNA Sequence | ORF Start: ATG at 14 | ORF Stop: end of sequence |

CACCGGATCCACCATGTGGCCGAGCCTGCTGCTGCTCCTGCTGTTGCCGGGGCCCCCGCCCGTCGCCG
GCTTGGAAGACGCTGCCTTCCCCCACCTGGGGGAGAGCTTGCAGCCCCTGCCCCGGGCCTGTCCCCTG
CGCTGCTCCTGCCCCCGAGTCGACACTGTGGACTGTGATGGCTTGGACCTTCGAGTGTTCCCGGACAA
CATCACCAGAGCCGCTCAGCACCTCTCCCTGCAGAACAACCAGCTCCAGGAACTCCCCTACAATGAGC
TGTCCCGCCTCAGTGGCCTGCGAACCCTCAACCTCCACAACAACCTCATCTCCTCCGAAGGCCTGCCT
GACGAGGCCTTCGAGTCCCTCACCCAGCTGCAGCACCTCTGCGTGGCTCACAACAAGCTCTCAGTGGC
CCCTCAGTTTCTGCCCCGGTCCCTCCGTGTCGCGGATCTGGCTGCCAACCAAGTGATGGAGATCTTCC
CCCTCACCTTTGGGGAGAAGCCGGCACTCAGGTCCGTGTACCTCCACAACAACCAGCTGAGCAACGCT
GGCCTGCCCCCCGACGCCTTCCGCGGCTCCGAGGCCATCGCCACCCTCAGCCTCTCCAACAACCAGCT
CAGCTACCTGCCGCCCAGCCTGCCGCCCTCACTCGAGCGGCTCCACCTGCAGAACAATCTCATCTCCA
AGGTGCCCCGAGGAGCCCTGAGCCGCCAGACTCAACTCCGTGAGCTCTACCTCCAGCACAACCAGCTG
ACAGACAGTGGCCTGGATGCCACCACCTTCAGCAAGCTGCATAGCCTTGAATACCTGGATCTCTCCCA
CAACCAGCTGACCACAGTGCCCGCCGGCCTGCCCCGGACCCTGGCTATCCTGCACCTGGGCCGCAACC
GCATCCGGCAGGTGGAGGCGGCTCGGCTGCACGGGGCGCGTGGTCTGCGCTATTTGTTGCTGCAGCAC
AACCAGCTGGGGAGCTCAGGGCTGCCCGCCGGGGCTCTGCGGCCGCTGCGGGGCCTGCACACGCTGCA
CCTCTATGGCAATGGGCTGGACCGCGTGCCTCCAGCCCTGCCCCGCCGCCTGCGTGCCCTGGTGCTGC
CCCACAACCACGTGGCCGCGCTGGGTGCCCGTGACCTGGTCGCCACACCGGGCCTGACGGAGCTTAAC
CTGGCCTATAACCGCCTGGCCAGCGCCCGTGTGCACCACCGGGCCTTCCGCCGGTTGCGTGCCCTGCG
CAGCCTCGACCTGGCAGGGAATCAGCTAACCCGGCTGCCCATGGGCCTGCCCACTGGCCTGCGCACCC
TGCAGCTGCAACGCAACCAGCTGCGGATGCTCGAGCCCGAGCCTCTGGCCGGCCTGGACCAACTGCGG
GAGCTCAGCCTGGCGCACAACCGGCTCCGGGTCGGCGACATCGGGCCAGGCACCTGGCATGAGCTCCA
AGCCCTCCAGATGCTGGACCTCAGCCACAATGAGCTGTCCTTTGTGCCCCCGGACCTGCCTGAGGCCC
TGGAGGAGCTGCACCTCGAGGGCAACCGCATCGGCCACGTGGGCCCCGAGGCCTTCCTCAGCACACCC
CGCCTGCGTGCCCTCTTCCTCAGGGCCAACAGGCTTCACATGACGAGCATCGCGGCTGAGGCCTTCCT
GGGGCTCCCAAACCTGCGTGTGGTGGACACGGCAGGGAATCCGGAGCAGGTCCTGATCCGGCTGCCTC
CCACCACCCCACGTGGGCCACGGGCAGGGGGCCCCAAGCTTGGC

| | | | |
|---|---|---|---|
| NOV42m, CG56904-08 | SEQ ID NO:996 | 577 aa | MW at 63582.4 kD |
| Protein Sequence | | | |

MWPSLLLLLLLPGPPPVAGLEDAAFPHLGESLQPLPRACPLRCSCPRVDTVDCDGLDLRVFPDNITRA
AQHLSLQNNQLQELPYNELSRLSGLRTLNLHNNLISSEGLPDEAFESLTQLQHLCVAHNKLSVAPQFL
PRSLRVADLAANQVMEIFPLTFGEKPALRSVYLHNNQLSNAGLPPDAFRGSEAIATLSLSNNQLSYLP
PSLPPSLERLHLQNNLISKVPRGALSRQTQLRELYLQHNQLTDSGLDATTFSKLHSLEYLDLSHNQLT
TVPAGLPRTLAILHLGRNRIRQVEAARLHGARGLRYLLLQHNQLGSSGLPAGALRPLRGLHTLHLYGN
GLDRVPPALPRRLRALVLPHNHVAALGARDLVATPGLTELNLAYNRLASARVHHRAFRRLRALRSLDL
AGNQLTRLPMGLPTGLRTLQLQRNQLRMLEPEPLAGLDQLRELSLAHNRLRVGDIGPGTWHELQALQM
LDLSHNELSFVPPDLPEALEELHLEGNRIGHVGPEAFLSTPRLRALFLRANRLHMTSIAAEAFLGLPN
LRWDTAGNPEQVLIRLPPTTPRGPRAGGPKLG

A ClustalW comparison of the above protein sequences yields the following sequence alignment shown in Table 42B.

TABLE 42B
Comparison of the NOV42 protein sequences.

| | | |
|---|---|---|
| NOV42a | ---MGRPTQWPSLLLLLLLPGPPPVAGLEDAAFPHLGESLQPLPRACPLRCSCPRVDTVD | (SEQ ID NO:972) |
| NOV42b | GSTMGRPTQWPSLLLLLLLPGPPPVAGLEDAAFPHLGESLQPLPRACPLRCSCPRVDTVD | (SEQ ID NO:974) |
| NOV42c | -----------------------------------------------------------I | (SEQ ID NO:976) |
| NOV42d | --------MWPSLLLLLLLPGPPPVAGLEDAAFPHLGESLQPLPRACPLRCSCPRVDTVD | (SEQ ID NO:978) |
| NOV42e | ----TGSTMWPSLLLLLLLPGPPPVAGLEDAAFPHLGESLQPLPRACPLRCSCPRVDTVD | (SEQ ID NO:980) |
| NOV42f | ----TGSTMWPSLLLLLLLPGPPPvAGLEDAAFPHLGESLQPLPRACPLRCSCPRVDTVD | (SEQ ID NO:982) |
| NOV42g | ----TGSTMWPSLLLLLLLPGPPPVAGLEDAAFPHLGESLQPLPRACPLRCSCPRVDTVD | (SEQ ID NO:984) |
| NOV42h | ------------------------------------------------------------ | (SEQ ID NO:986) |
| NOV42i | ------------------------------------------------------------ | (SEQ ID NO:988) |
| NOV42j | --------MWPSLLLLLLLPGPPPVAGLEDAAFPHLGESLQPLPRACPLRCSCPRVDTVD | (SEQ ID NO:990) |
| NOV42k | ---MGRPTQWPSLLLLLLLPGPPPVAGLEDAAFPHLGESLQPLPRACPLRCSCPRVDTVD | (SEQ ID NO:992) |
| NOV42l | ---MGRPTQWPSLLLLLLLPGPPPVAGLEDAAFPHLGESLQPLPRACPLRCSCPRVDTVD | (SEQ ID NO:994) |
| NOV42m | --------MWPSLLLLLLLPGPPPVAGLEDAAFPHLGESLQPLPRACPLRCSCPRVDTVD | (SEQ ID NO:996) |
| NOV42a | CDGLDLRVFPDNITRAAQHLSLQNNQLQELPYNELSRLSGLRTLNLHNNLISSEGLPDEA | |
| NOV42b | CDGLDLRVFPDNITRAAQHLSLQNNQLQELPYNELSRLSGLRTLNLHNNLISSEGLPDEA | |
| NOV42c | HHGLDLRVFPDNITPAAQHLSLQNNQLQELPYNELSRLSGLRTLNLHNNLISSEGLPDEA | |
| NOV42d | CDGLDLRVFPDNITRAAQHLSLQNNQLQELPYNELSRLSGLRTLNLHNNLISSEGLPDEA | |
| NOV42e | CDGLDLRVFPDNITRAAQHLSLQNNQLQELPYNELSRLSGLRTLNLHNNLISSEGLPDEA | |
| NOV42f | CDGLDLRVFPDNITPAAQHLSLQNNQLQELPYNELSRLSGLRTLNLHNNLISSEGLPDEA | |
| NOV42g | CDGLDLRVFPDNITRAAQHLSLQNNQLQELPYNELSRLSGLRTLNLHNNLISSEGLPDEA | |
| NOV42h | ---------------------MMNNQLQELPYNELSRLSGLRTLNLHNNLISSEGLPDEA | |
| NOV42i | ---------------------MMNNQLQELPYNELSRLSGLRTLNLHNNLISSEGLPDEA | |
| NOV42j | CDGLDLRVFPDNITRAAQHLSLQNNQLQELPYNELSRLSGLRTLNLHNNLISSEGLPDEA | |
| NOV42k | CDGLDLRVFPDNITRAAQHLSLQNNQLQELPYNELSRLSGLRTLNLHNNLISSEGLPDEA | |
| NOV42l | CDGLDLRVFPDNITRAAQHLSLQNNQLQELPYNELSRLSGLRTLNLHNNLISSEGLPDEA | |
| NOV42m | CDGLDLRVFPDNITRAAQHLSLQNNQLQELPYNELSRLSGLRTLNLHNNLISSEGLPDEA | |
| NOV42a | FESLTQLQHLCVAHNK-------------------------------------------- | |
| NOV42b | FESLTQLQHLCVAHNK-------------------------------------------- | |
| NOV42c | FESLTQLQHLCVAHNK-------------------------------------------- | |
| NOV42d | FESLTQLQHLCVAHNKLSVAPQFLPRSLRVADLAANQVMEIFPLTFGEKPALRSVYLHNN | |
| NOV42e | FESLTQLQHLCVAHNKLSVAPQFLPRSLR------------------------------- | |
| NOV42f | FESLTQLQHLCVAHNKLSVAPQFLPRSLRVADLAANQVMEIFPLTFGEKPALRSVYLHNN | |
| NOV42g | FESLTQLQHLCVAHNKLSVAPQFLPRSLRVADLAANQVMEIFPLTFGEKPALRSVYLHNN | |
| NOV42h | FESLTQLQHLCVAHNKLSVAPQFLPRSLRVADLAANQVMEIFPLTFGEKPALRSVYLHNN | |
| NOV42i | FESLTQLQHLCVAHN--------------------------------------------- | |
| NOV42j | FESLTQLQHLCVAHNK-------------------------------------------- | |
| NOV42k | FESLTQLQHLCVAHNKLSVAPQFLPRSLRVADLAANQVMEIFPLTFGEKPALRSVYLHNN | |
| NOV42l | FESLTQLQHLCVAHNK-------------------------------------------- | |
| NOV42m | FESLTQLQHLCVAHNKLSVAPQFLPRSLRVADLAANQVMEIFPLTFGEKPALRSVYLHNN | |
| NOV42a | ---------------------------------------------NNLISKVPRGALSRQ | |
| NOV42b | ---------------------------------------------NNLISKVPRGALSRQ | |
| NOV42c | ---------------------------------------------NNLISKVPRGALSRQ | |
| NOV42d | QLSNAGLPPDAFRGSEAIATLSLSNNQLSYLPPSLPPSLERLHLQNNLISKVPRGALSRQ | |
| NOV42e | ----------------------------------------VADLAANQVMEIFPLTFGEK | |
| NOV42f | QLSNAGLPPDAFRGSEAIATLSLSNNQLSYLPPSLPPSLERLHLQNNLISKVPRGALSRQ | |
| NOV42g | QLSNAGLPPDAFRGSEAIATLSLSNNQLSYLPPSLPPSLERLHLQNNLISKVPRGALSRQ | |
| NOV42h | QLSNAGLPPDAFRGSEAIATLSLSNNQLSYLPPSLPPSLERLHLQNNLISKVPRGALSRQ | |
| NOV42i | ------------------------K--------------------NNLISKVPRGALSRQ | |
| NOV42j | ---------------------------------------------NNLISKVPRGALSRQ | |
| NOV42k | QLSNAGLPPDAFRGSEAIATLSLSNNQLSYLPPSLPPSLERLHLQNNLISKVPRGALSRQ | |
| NOV42l | ---------------------------------------------NNLISKVPRGALSRQ | |
| NOV42m | QLSNAGLPPDAFRGSEAIATLSLSNNQLSYLPPSLPPSLERLHLQNNLISKVPRGALSRQ | |
| NOV42a | TQLRELYLQHNQLTDSGLDATTFSKLHSLEYLDLSHNQLTTVPAGLPRTLAILHLGRNRI | |
| NOV42b | TQLRELYLQHNQLTDSGLDATTFSKLHSLEYLDLSHNQLTTVPAGLPRTLAILHLGRNRI | |
| NOV42c | TQLRELYLQHNQLTDSGLDATTFSKLHSLEYLDLSHNQLTTVPAGLPRTLAILHLGRNRI | |
| NOV42d | TQLRELYLQHNQLTDSGLDATTFSKLHSLEYLDLSHNQLTTVPAGLPRTLAILHLGRNRI | |
| NOV42e | PALRSVYLHNNQLSNAGLPPDAFRGSEAIATLSLSNNQLSYLPPSLPPSLERLHLQNNLI | |
| NOV42f | TQLRELYLQHNQLTDSGLDATTFSKLHSLEYLDLSHNQLTTVPAGLPRTLAILHLGRNRI | |
| NOV42g | TQLRELYLQHNQLTDSGLDATTFSKLHSLEYLDLSHNQLTTVPAGLPRTLAILHLGRNRI | |
| NOV42h | TQLRELYLQHNQLTDSGLDATTFSKLHSLEYLDLSHNQLTTVPAGLPRTLAILHLGPNRI | |
| NOV42i | TQLRELYLQHNQLTDSGLDATTFSKLHSLEYLDLSHNQLTTVPAGLPRTLAILHLGRNRI | |
| NOV42j | TQLRELYLQHNQLTDSGLDATTFSKLHSLEYLDLSHNQLTTVPAGLPRTLAILHLGRNRI | |
| NOV42k | TQLRELYLQHNQLTDSGLDATTFSKLHSLEYLDLSHNQLTTVPAGLPRTLAILHLGRNRI | |
| NOV42l | TQLRELYLQHNQLTDSGLDATTFSKLHSLEYLDLSHNQLTTVPAGLPRTLAILHLGRNRI | |
| NOV42m | TQLRELYLQHNQLTDSGLDATTFSKLHSLEYLDLSHNQLTTVPAGLPRTLAILHLGRNRI | |
| NOV42a | RQVEAARLHGARGLRYLLLQHNQLGSSGLPAGALRPLRGLHTLHLDGNGLDRVPPALPRR | |
| NOV42b | RQVEAARLHGARGLRYLLLQHNQLGSSGLPAGALRPLRGLHTLHLYGNGLDRVPPALPRR | |
| NOV42c | RQVEAARLHGARGLRYLLLQHNQLGSSGLPAGALRPLRGLHTLHLYGNGLDRVPPALPRR | |
| NOV42d | RQVEAARLHGARGLRYLLLQHNQLGSSGLPAGALRPLRGLHTLHLYGNGLDRVPPALPRR | |
| NOV42e | SKVPRGALSRQTQLRELYLQHNQLTDSGLDATTFSKLHSLEYLDLSHNQLTTVPAGLPRT | |
| NOV42f | RQVEAARLHGARGLRYLLLQHNQLGSSGLPAGALRPLRGLHTLHLYGNGLDRVPPALPRR | |
| NOV42g | RQVEAARLHGARGLRYLLLQHNQLGSSGLPAGALRPLRGLHTLHLYGNGLDRVPPALPRR | |
| NOV42h | RQVEAARLHGARGLRYLLLQHNQLGSSGLPAGALRPLRGLHTLHLYGNGLDRVPPALPRR | |
| NOV42i | RQVEAARLHGARGLRYLLLQHNQLGSSGLPAGALRPLRGLHTLHLYGNGLDRVPPALPRR | |
| NOV42j | RQVEAARLHGARGLRYLLLQHNQLGSSGLPAGALRPLRGLHTLHLYGNGLDRVPPALPRR | |
| NOV42k | RQVEAARLHGARGLRYLLLQHNQLGSSGLPAGALRPLRGLHTLHLYGNGLDRVPPALPRR | |
| NOV42l | RQVEAARLHGARGLRYLLLQHNQLGSSGLPAGALRPLRGLHTLHLYGNGLDRVPPALPRR | |
| NOV42m | RQVEAARLHGARGLRYLLLQHNQLGSSGLPAGALRPLRGLHTLHLYGNGLDRVPPALPRR | |
| NOV42a | LRALVLPHNHVAALGARDLVATPGLTELNLAYNRLASARVHHRAFRRLRALRSLDLAGNQ | |
| NOV42b | LRALVLPHNHVAALGARDLVATPGLTELNLAYNRLASARVHHRAFRRLRALRSLDLAGNQ | |
| NOV42c | LRALVLPHNHVAALGARDLVATPGLTELNLAYNRLASARVHHRAFRRLHALRSLDLAGNQ | |
| NOV42d | LRALVLPHNHVAALGARDLVATPGLTELNLAYNRLASARVHHRAFRRLRALRSLDLAGNQ | |
| NOV42e | LAILHLGR---------------------------------------NRIRQMLDLSHNE | |
| NOV42f | LRALVLPHNHVAALGARDLVATPGLTELNLAYNRLASARVHHRAFRRLRALRSLDLAGNQ | |
| NOV42g | LRALVLPHNHVAALGARDLVATPGLTELNLAYNRLASARVHHRAFRRLRALRSLDLAGNQ | |
| NOV42h | LRALVLPHNHVAALGARDLVATPGLTELNLAYNRLASARVHHRAFRRLRALRSLDLAGNQ | |
| NOV42i | LRALVLPHNHVAALGARDLVATPGLTELNLAYNRLASARVHHRAFPRLRALRSLDLAGNQ | |
| NOV42l | LRALVLFHNHVAALGARDLVATFGLTELNLAYNRLASARVHHRAFRRLRALRSLDLAGNQ | |
| NOV42k | LRALVLPHNHVAALGARDLVATPGLTELNLAYNRLASARVHHEAFRRLRALRSLDLAGNQ | |
| NOV421 | LRALVLPHNHVAALGARDLVATPGLTELNLAYNRLASARVHHRAFRRLEALRSLDLAGNQ | |
| NOV42m | LRALVLPHNHVAALGARDLVATPGLTELNLAYNRLASARVHHPAFRRLHALRSLDLAGNQ | |
| NOV42a | LTRLPMGLPTGLRTLQLQRNQLRMLEPEPLAGLDQLRELSLAHNRLRVGDIGPGTWHELQ | |
| NOV42b | LTRLPMGLPTGLRTLQLQRNQLRMLEPEPLAGLDQLRELSLAHNRLRVGDIGFGTWHELQ | |
| NOV42c | LTRLPMGLPTGLRTLQLQRNQLRMLEPEPLAGLDQLRELSLAHNRLRVGDIGPGTWHELQ | |
| NOV42d | LTRLPMGLPTGLRTLQLQRNQLRMLEPEPLAGLDQLRELSLAHNRLRVGDIGPGTWHELQ | |
| NOV42e | LSFVPPDLPEALEELHLEGNRIGHVGPEAFLSTPRLRALFLRANRLHMTSIAAEAFLGLP | |
| NOV42f | LTRLPMGLPTGLRTLQLQRNQLRMLEPEPLAGLDQLRELSLAHNRLRVGDIGPGTWHELQ | |
| NOV42g | LTRLPMGLPTGLRTLQLQRNQLRMLEPEPLAGLDQLRELSLAHNRLRVGDIGPGTWHELQ | |
| NOV42h | LTRLPMGLPTGLRTLQLQRNQLRMLEPEPLAGLDQLRELSLAHNRLRVGDIGPGTWHELQ | |
| NOV42i | LTRLPMGLPTGLRTLQLQRNQLRMLEPEPLAGLDQLRELSLAHNRLRVGDIGPGTWHELQ | |
| NOV42j | LTRLPMGLPTGLRTLQLQRNQLRMLEPEPLAGLDQLRELSLAHNRLRVGDIGPGTWHELQ | |
| NOV42k | LTRLPMGLPTGLRTLQLQRNQLRMLEPEPLAGLDQLRELSLAHNRLRVGDIGPGTWHELQ | |
| NOV42l | LTRLPMGLPTGLRTLQLQRNQLRMLEPEPLAGLDQLRELSLAHNRLRVGDIGPGTWHELQ | |
| NOV42m | LTRLPMGLPTGLRTLQLQRNQLRMLEPEPLAGLDQLRELSLAHNRLRVGDIGPGTWHELQ | |
| NOV42a | ALQVRHRLVSHT--VPRAPPSPCLPCHVPNILVSW------------------------- | |
| NOV42b | ALQVRHRLVSHT--VPRAPPSPCLPCHVPNILVSWEF----------------------- | |
| NOV42c | ALQVRHRLVSHT--VPRAPPSPCLPCHVPNILVSWEFKGEFQA----------------- | |
| NOV42d | ALQMLDLSHNELSFVPPDLPEALEELHLEGNRIGHVGPEAFLSTPRLRALFLRANRLHMT | |
| NOV42e | NLRVVDTAGNPEQVLIRLPPTTPRGPRAGGPKLG-------------------------- | |
| NOV42f | ALQMLDLSHNELSFVPPDLPEALEELHLEGNRIGHVGPEAFLSTPRLRALFLRANRLHMT | |
| NOV42g | ALQMLDLSHNELSFVPPDLPEALEELHLEGNRIGHVGPEAFLSTPRLRALFLRANRLHMT | |
| NOV42h | ALQMLDLSHNELSFVPPDLPEALEELHLEGNRIGHVGPEAFLSTPRLRALFLRANRLHMT | |
| NOV42i | ALQMLDLSHNELSFVPPDLPEALEELHLEGNRIGHVGPEAFLSTPRLRALFLRANRLHMT | |
| NOV42j | ALQMLDLSHNELSFVPPDLPEALEELHLEGNRIGHVGPEAFLSTPRLRALFLRANRLHMT | |
| NOV42k | ALQMLDLSHNELSFVPPDLPEALEELHLEGNRIGHVGPEAFLSTPRLRALFLRANRLHMT | |
| NOV42l | ALQVRHRLVSHT--VPEAPPSPCLPCHVPNILVSW------------------------- | |
| NOV42m | ALQMLDLSHNELSFVPPDLPEALEELHLEGNRIGHVGPEAFLSTPRLRALFLRANRLHMT | |
| NOV42a | --------------------------------------------- | |
| NOV42c | --------------------------------------------- | |
| NOV42d | SIAAEAFLGLPNLRVVDTAGNPEQVLIRLPPTTPRGPRAGGP--- | |
| NOV42e | --------------------------------------------- | |
| NOV42f | SIAAEAFLGLPNLRVVDTAGNPEQVLIRLPPTTPRGPEAGGPKLG | |
| NOV42g | SIAAEAFLGLPNLRVVDTAGNPEQVLIRLPPTTPRGPRAGGPEFG | |
| NOV42h | SIAAEAFLGLPNLRVVDTAGNPEQVLIRLPPTTPRGPRAGGP--- | |
| NOV42i | SIAAEAFLGLPNLRVVDTAGNPEQVLIRLPPTTPRGPRAGGP--- | |
| NOV42j | SIAAEAFLGLPNLRVVDTAGNPEQVLIRLPPTTPRGPRAGGP--- | |
| NOV42k | SIAAEAFLGLPNLRVVDTAGNPEQVLIRLPPTTPRGPRAGGP--- | |
| NOV42l | --------------------------------------------- | |
| NOV42m | SIAAEAFLGLPNLRVVDTAGNPEQVLIRLPPTTPRGPRAGGPKLG | |

Further analysis of the NOV42a protein yielded the following properties shown in Table 42C.

TABLE 42C
Protein Sequence Properties NOV42a

| | |
|---|---|
| SignalP analysis: | Cleavage site between residues 25 and 26 |
| PSORT II analysis: | |

| PSG: | a new signal peptide prediction method | |
|---|---|---|
| | N-region: | length 3; pos. chg 1; neg. chg 0 |
| | H-region: | length 22; peak value 10.55 |
| | PSG score: | 6.15 |

| | |
|---|---|
| GvH: | von Heijne's method for signal seq. recognition |
| | GvH score (threshold: −2.1): 2.50 |
| | possible cleavage site: between 24 and 25 |

>>> Seems to have a cleavable signal peptide (1 to 24)

| ALOM: | Klein et al's method for TM region allocation | |
|---|---|---|
| | Init position for calculation: 25 | |
| | Tentative number of TMS(s) for the threshold 0.5: 0 | |
| | number of TMS(s) . . . fixed | |
| | PERIPHERAL | Likelihood = 4.77 (at 187) |

| | ALOM score: 4.77 (number of TMSs: 0) | | | |
|---|---|---|---|---|
| MTOP: | Prediction of membrane topology (Hartmann et al.) | | | |
| | Center position for calculation: 12 | | | |
| | Charge difference: −4.5 C(−2.5)-N(2.0) | | | |
| | N >= C: N-terminal side will be inside | | | |
| MITDISC: | discrimination of mitochondrial targeting seq | | | |
| | R content: | 1 | Hyd Moment(75): | 6.51 |
| | Hyd Moment(95): | 8.64 | G content: | 3 |
| | D/E content: | 1 | S/T content: | 2 |

| | |
|---|---|
| | Score: −4.56 |
| Gavel: | prediction of cleavage sites for mitochondrial preseq |
| | R-2 motif at 13 GRP\|TQ |

NUCDISC: discrimination of nuclear localization signals

| | |
|---|---|
| | pat4: none |
| | pat7: PRRLRAL (5) at 266 |
| | bipartite: none |
| | content of basic residues: 10.7% |
| | NLS Score: −0.04 |
| KDEL: | ER retention motif in the C-terminus: none |

ER Membrane Retention Signals:

XXRR-like motif in the N-terminus: GRPT

none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs:

```
Leucine zipper pattern (PS00029): *** found ***
  LDLSHNQLTTVPAGLPRTLAIL at 180
  LHLDGNGLDRVPPALPRRLRAL at 251
  LDLAGNQLTRLPMGLPTGLRTL at 322
```

none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear |
| | discrimination |
| | Prediction: nuclear |
| | Reliability: 89 |
| COIL: | Lupas's algorithm to detect coiled-coil regions |
| | total: 0 residues |

Final Results (k = 9/23):

77.8%: extracellular, including cell wall
11.1%: vacuolar
11.1%: mitochondrial

>> prediction for CG56904-01 is exc (k = 9)

A search of the NOV42a protein against the Geneseq database, a proprietary database that contains sequences published in patents and patent publication, yielded several homologous proteins shown in Table 42D.

TABLE 42D
Geneseq Results for NOV42a

| | | NOV42a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAU91336 | Human novel secreted protein | 7 . . . 421 | 414/415 (99%) | 0.0 |
| | LP255(a) - Homo sapiens, 423 aa. | 9 . . . 423 | 414/415 (99%) | |
| | [WO200214358-A2, 21-FEB-2002] | | | |
| AAU91340 | Human novel secreted protein | 7 . . . 421 | 413/459 (89%) | 0.0 |
| | LP255(b) - Homo sapiens, 467 aa. | 9 . . . 467 | 413/459 (89%) | |
| | [WO200214358-A2, 21-FEB-2002] | | | |
| AAG65893 | Amino acid sequence of GSK gene Id | 4 . . . 405 | 315/494 (63%) | e−153 |
| | 90060 - Homo sapiens, 605 aa. | 30 . . . 523 | 336/494 (67%) | |
| | [WO200172961-A2, 04-OCT-2001] | | | |
| AAG65892 | Amino acid sequence of GSK gene Id | 63 . . . 405 | 283/364 (77%) | e−152 |
| | 90060 - Homo sapiens, 483 aa. | 38 . . . 401 | 300/364 (81%) | |
| | [WO200172961-A2, 04-OCT-2001] | | | |
| ABU54399 | Human secreted/transmembrane | 39 . . . 392 | 178/443 (40%) | 6e−79 |
| | protein PRO332 - Homo sapiens, 642 | 92 . . . 534 | 232/443 (52%) | |
| | aa. [US2002132240-A1, 19-SEP-2002] | | | |

In a BLAST search of public sequence databases, the NOV42a protein was found to have homology to the proteins shown in the BLASTP data in Table 42E.

TABLE 42E
Public BLASTP Results for NOV42a

| | | NOV42a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAD29040 | Sequence 27 from Patent | 7 . . . 421 | 414/415 (99%) | 0.0 |
| | WO0214358 - Homo sapiens | 9 . . . 423 | 414/415 (99%) | |
| | (Human), 423 aa. | | | |
| CAD29044 | Sequence 35 from Patent | 7 . . . 421 | 413/459 (89%) | 0.0 |
| | WO0214358 - Homo sapiens | 9 . . . 467 | 413/459 (89%) | |
| | (Human), 467 aa. | | | |
| Q9H5G9 | Hypothetical protein FLJ23447 - | 131 . . . 421 | 290/291 (99%) | e−166 |
| | Homo sapiens (Human), 363 aa. | 73 . . . 363 | 290/291 (99%) | |
| Q8N641 | Similar to hypothetical protein | 131 . . . 392 | 141/262 (53%) | 4e−70 |
| | FLJ23447 - Homo sapiens (Human), | 33 . . . 294 | 175/262 (65%) | |
| | 402 aa. | | | |
| Q8BM45 | Hypothetical leucine-rich | 131 . . . 392 | 140/262 (53%) | 5e−70 |
| | repeat/leucine-rich repeat - Mus | 33 . . . 294 | 176/262 (66%) | |
| | musculus (Mouse), 399 aa. | | | |

PFam analysis predicts that the NOV42a protein contains the domains shown in the Table 42F.

TABLE 42F
Domain Analysis of NOV42a

| | | Identities/ | |
|---|---|---|---|
| Pfam | NOV42a | Similarities for | Expect |
| Domain | Match Region | the Matched Region | Value |
| LRRNT | 43 . . . 71 | 11/35 (31%) | 0.24 |
| | | 22/35 (63%) | |
| LRR | 97 . . . 122 | 11/26 (42%) | 0.0047 |
| | | 23/26 (88%) | |
| LRR | 150 . . . 175 | 12/26 (46%) | 0.056 |
| | | 25/26 (96%) | |
| LRR | 176 . . . 195 | 11/25 (44%) | 0.0079 |
| | | 20/25 (80%) | |
| LRR | 221 . . . 246 | 10/26 (38%) | 0.47 |
| | | 22/26 (85%) | |
| LRR | 363 . . . 388 | 10/26 (38%) | 0.46 |
| | | 22/26 (85%) | |

### Example 43

The NOV43 clone was analyzed, and the nucleotide and encoded polypeptide sequences are shown in Table 43A.

TABLE 43A
NOV43 Sequence Analysis

| | | |
|---|---|---|
| NOV43a, CG56914-01 | SEQ ID NO:997 | 2153 bp |
| DNA Sequence | ORF Start: ATG at 16 | ORF Stop: TAA at 2137 |

GATGCTGGCACTTACATGTGTGTGGCCCAGAACCCGGCTGGTACAGCCTTGGGCAAAATCAAGTTAAA
TGTCCAAGTTCCTCCAGTCATTAGCCCTCATCTAAAGGAATATGTTATTGCTGTGGACAAGCCCATCA
CGTTATCCTGTGAAGCAGATGGCCTCCCTCCGCCTGACATTACATGGCATAAAGATGGGCGTGCAATT
GTGGAATCTATCCGCCAGCGCGTCCTCAGCTCTGGCTCTCTGCAAATAGCATTTGTCCAGCCTGGTGA
TGCTGGCCATTACACGTGCATGGCAGCCAATGTAGCAGGATCAAGCAGCACAAGCACCAAGCTCACCG
TCCATGTACCACCCAGGATCAGAAGTACAGAAGGACACTACACGGTCAATGAGAATTCACAAGCCATT
CTTCCATGCGTAGCTGATGGAATCCCCACACCAGCAATTAACTGGAAAAAAGACAATGTTCTTTTAGC
TAACTTGTTAGGAAAATACACTGCTGAACCATATGGAGAACTCATTTTAGAAAATGTTGTGCTGGAGG
ATTCTGGCTTCTATACCTGTGTTGCTAACAATGCTGCAGGTGAAGATACACACACTGTCAGCCTGACT
GTGCATGTTCTCCCCACTTTTACTGAACTTCCTGGAGACGTGTCATTAAATAAAGGAGAACAGCTACG
ATTAAGCTGTAAAGCTACTGGTATTCCATTGCCCAAATTAACATGGACCTTCAATAACAATATTATTC
CAGCCCACTTTGACAGTGTGAATGGACACAGTGAACTTGTTATTGAAAGAGTGTCAAAAGAGGATTCA
GGTACTTATGTGTGCACCGCAGAGAACAGCGTTGGCTTTGTGAAGGCAATTGGATTTGTTTATGTGAA
AGAACCTCCAGTCTTCAAAGGTGATTATCCTTCTAACTGGATTGAACCACTTGGTGGGAATGCAATCC
TGAATTGTGAGGTGAAAGGAGACCCCACCCCAACCATCCAGTGGAACAGAAAGGGAGTGGATATTGAA
ATTAGCCACAGAATCCGGCAACTGGGCAATGGCTCCCTGGCCATCTATGGCACTGTTAATGAAGATGC
CGGTGACTATACATGTGTAGCTACCAATGAAGCTGGGGTGGTGGAGCGCAGCATGAGTCTGACTCTGC
AAAGTCCTCCTATTATCACTCTTGAGCCAGTGGAAACTGTTATTAATGCTGGTGGCAAAATCATATTG
AATTGTCAGGCAACTGGAGAGCCTCAACCAACCATTACATGGTCCCGTCAAGGGCACTCTATTTCCTG
GGATGACCGGGTTAACGTGTTGTCCAACAACTCATTATATATTGCTGATGCTCAGAAAGAAGATACCT
CTGAATTTGAATGCGTTGCTCGAAACTTAATGGGTTCTGTCCTTGTCAGAGTGCCAGTCATAGTCCAG
GTTCATGGTGGATTTTCCCAGTGGTCTGCATGGAGAGCCTGCAGTGTCACCTGTGGAAAAGGCATCCA
AAAGAGGAGTCGTCTGTGCAACCAGCCCCTTCCAGCCAATGGTGGGAAGCCCTGCCAAGGTTCAGATT
TGGAAATGCGAAACTGTCAAAATAAGCCTTGTCCAGTGGATGGTAGCTGGTCGGAATGGAGTCTTTGG
GAAGAATGCACAAGGAGCTGTGGACGCGGCAACCAAACCAGGACCAGGACTTGCAATAATCCATCAGT
TCAGCATGGTGGGCGGCCATGTGAAGGGAATGCTGTGGAAATAATTATGTGCAACATTAGGCCTTGCC
CAGTTCATGGAGCATGGAGCGCTTGGCAGCCTTGGGGAACATGCAGCGAAAGTTGTGGGAAAGGTACT
CAGACAAGAGCAAGACTTTGTAATAACCCACCACCAGCGTTTGGTGGGTCCTACTGTGATGGAGCAGA
AACACAGATGCAAGTTTGCAATGAAAGAAATTGTCCAATTCATGGCAAGTGGGCGACTTGGGCCAGTT
GGAGTGCCTGTTCTGTGTCATGTGGAGGAGGTGCCAGACAGAGAACAAGGGGCTGCTCCGACCCTGTG
CCCCAGTATGGAGGAAGGAAATGCGAAGGGAGTGATGTCCAGAGTGATTTTTGCAACAGTGACCCTTG
CCCAAGTGAGTGTTGGAAATACCCATGGTAACTGGAGTCCTTGGA

| | | | |
|---|---|---|---|
| NOV43a, CG56914-01 | SEQ ID NO:998 | 707 aa | MW at 76557.7 kD |
| Protein Sequence | | | |

MCVAQNPAGTALGKIKLNVQVPPVISPHLKEYVIAVDKPITLSCEADGLPPPDITWHKDGRAIVESIR
QRVLSSGSLQIAFVQPGDAGHYTCMAANVAGSSSTSTKLTVHVPPRIRSTEGHYTVNENSQAILPCVA
DGIPTPAINWKKDNVLLANLLGKYTAEPYGELILENVVLEDSGFYTCVANNAAGEDTHTVSLTVHVLP
TFTELPGDVSLNKGEQLRLSCKATGIPLPKLTWTFNNNIIPAHFDSVNGHSELVIERVSKEDSGTYVC
TAENSVGFVKAIGFVYVKEPPVFKGDYPSNWIEPLGGNAILNCEVKGDPTPTIQWNRKGVDIEISHRI
RQLGNGSLAIYGTVNEDAGDYTCVATNEAGVVERSMSLTLQSPPIITLEPVETVINAGGKIILNCQAT
GEPQPTITWSRQGHSISWDDRVNVLSNNSLYIADAQKEDTSEFECVARNLMGSVLVRVPVIVQVHGGF
SQWSAWRACSVTCGKGIQKRSRLCNQPLPANGGKPCQGSDLEMRNCQNKPCPVDGSWSEWSLWEECTR
SCGRGNQTRTRTCNNPSVQHGGRPCEGNAVEIIMCNIRPCPVHGAWSAWQPWGTCSESCGKGTQTRAR
LCNNPPPAFGGSYCDGAETQMQVCNERNCPIHGKWATWASWSACSVSCGGGARQRTRGCSDPVPQYGG
RKCEGSDVQSDFCNSDPCPSECWKYPW

| | | |
|---|---|---|
| NOV43b, 262802367 | SEQ ID NO:999 | 2136 bp |
| DNA Sequence | ORF Start: at 1 | ORF Stop: end of sequence |

GGATCCACCATGTGTGTGGCCCAGAACCCGGCTGGTACAGCCTTGGGCAAAATCAAGTTAAATGTCCA
AGTTCCTCCAGTCATTAGCCCTCATCTAAAGGAATATGTTATTGCTGTGGACAAGCCCATCACGTTAT
CCTGTGAAGCAGATGGCCTCCCTCCGCCTGACATTACATGGCATAAAGATGGGCGTGCAATTGTGGAA
TCTATCCGCCAGCGCGTCCTCAGCTCTGGCTCTCTGCAAATAGCATTTGTCCAGCCTGGTGATGCTGG
CCATTACACGTGCATGGCAGCCAATGTAGCAGGATCAAGCAGCACAAGCACCAAGCTCACCGTCCATG
TACCACCCAGGATCAGAAGTACAGAAGGACACTACACGGTCAATGAGAATTCACAAGCCATTCTTCCA
TGCGTAGCTGATGGAATCCCCACACCAGCAATTAACTGGAAAAAAGACAATGTTCTTTTAGCTAACTT
GTTAGGAAAATACACTGCTGAACCATATGGAGAACTCATTTTAGAAAATGTTGTGCTGGAGGATTCTG
GCTTCTATACCTGTGTTGCTAACAATGCTGCAGGTGAAGATACACACACTGTCAGCCTGACTGTGCAT
GTTCTCCCCACTTTTACTGAACTTCCTGGAGACGTGTCATTAAATAAAGGAGAACAGCTACGATTAAG
CTGTAAAGCTACTGGTATTCCATTGCCCAAATTAACATGGACCTTCAATAACAATATTATTCCAGCCC
ACTTTGACAGTGTGAATGGACACAGTGAACTTGTTATTGAAAGAGTGTCAAAAGAGGATTCAGGTACT
TATGTGTGCACCGCAGAGAACAGCGTTGGCTTTGTGAAGGCAATTGGATTTGTTTATGTGAAAGAACC
TCCAGTCTTCAAAGGTGATTATCCTTCTAACTGGATTGAACCACTTGGTGGGAATGCAATCCTGAATT
GTGAGGTGAAAGGAGACCCCACCCCAACCATCCAGTGGAACAGAAAGGGAGTGGATATTGAAATTAGC
CTATACATGTGTAGCTACCAATGAAGCTGGGGTGGTGGAGCGCAGCATGAGTCTGACTCTGCGAAGTC
CTCCTATTATCACTCTTGAGCCAGTGGAAACTGTTATTAATGCTGGTGGCAAAATCATATTGAATTGT
CAGGCAACTGGAGAGCCTCAACCAACTATTACATGGTCCCGTCAAGGGCACTCTATTTCCTGGGATGA
CCGGGTTAACGTGTTGTCCAACAACTCATTATATATTGCTGATGCTCAGAAAGAAGATACCTCTGAAT
TTGAATGTGTTGCTCGAAACTTAATGGGTTCTGTCCTTGTCAGAGTGCCAGTCATAGTCCAGGTTCAT
GGTGGATTTTCCCAGTGGTCTGCATGGAGAGCCTGCAGTGTCACCTGTGGAAAAGGCATCCAAAAGAG
TGCGAAACTGTCAAAATAAGCCTTGTCCAGTGGATGGTAGCTGGTCGGAATGGAGTCTTTGGGAAGAA
TGCACAAGGAGCTGTGGACGCGGCAACCAAACCAGGACCAGGACTTGCAATAATCCATCAGTTCAGCA
TGGTGGGCGGCCATGTGAAGGGAATGCTGTGGAAATAATTATGTGCAACATTAGGCCTTGCCCAGTTC
ATGGAGCATGGAGCGCTTGGCAGCTTTGGGGAACATGCAGCGAAAGTTGTGGGAAAGGTACTCAGACA
AGAGCAAGACTTTGTAATAACCCACCACCAGCGTTTGGTGGGTCCTACTGTGATGGAGCAGAAACACA
GATGCAAGTTTGCAATGAAAGAAATTGTCCAATTCATGGCAAGTGGGCGACTTGGGCCAGTTGGAGTG
CCTGTTCTGTGTCATGTGGAGGAGGTGCCAGACAGAGAACAAGGGGCTGCTCCGACCCTGTGCCCCAG
TATGGAGGAAGGAAATGCGAAGGGAGTGATGTCCAGAGTGATTTTTGCAACAGTGACCCTTGCCCAAG
TGAGTGTTGGAAATACCCATGGCTCGAG

| | | | |
|---|---|---|---|
| NOV43b, 262802367 | SEQ ID NO:1000 | 712 aa | MW at 77089.2 kD |
| Protein Sequence | | | |

GSTMCVAQNPAGTALGKIKLNVQVPPVISPHLKEYVIAVDKPITLSCEADGLPPPDITWHKDGRAIVE
SIRQRVLSSGSLQIAFVQPGDAGHYTCMAANVAGSSSTSTKLTVHVPPRIRSTEGHYTVNENSQAILP
CVADGIPTPAINWKKDNVLLANLLGKYTAEPYGELILENVVLEDSGFYTCVANNAAGEDTHTVSLTVH
VLPTFTELPGDVSLNKGEQLRLSCKATGIPLPKLTWTFNNNIIPAHFDSVNGHSELVIERVSKEDSGT
YVCTAENSVGFVKAIGFVYVKEPPVFKGDYPSNWIEPLGGNAILNCEVKGDPTPTIQWNRKGVDIEIS
HRIRQLGNGSLAIYGTVNEDAGDYTCVATNEAGVVERSMSLTLRSPPIITLEPVETVINAGGKIILNC
QATGEPQPTITWSRQGHSISWDDRVNVLSNNSLYIADAQKEDTSEFECVARNLMGSVLVRVPVIVQVH
GGFSQWSAWRACSVTCGKGIQKRSRLCNQPLPANGGKPCQGSDLEMRNCQNKPCPVDGSWSEWSLWEE
CTRSCGRGNQTRTRTCNNPSVQHGGRPCEGNAVEIIMCNIRPCPVHGAWSAWQLWGTCSESCGKGTQT
RARLCNNPPPAFGGSYCDGAETQMQVCNERNCPIHGKWATWASWSACSVSCGGGARQRTRGCSDPVPQ
YGGRKCEGSDVQSDFCNSDPCPSECWKYPWLE

| | | |
|---|---|---|
| NOV43c, CG56914-02 | SEQ ID NO:1001 | 15660 bp |
| DNA Sequence | ORF Start: ATG at 1649 | ORF Stop: TAA at 15134 |

GATTAGTGGCATAAACTGTAGGTCAGCTGGTGGAGGCAAGCCAGCAAGGGGCTTCATGGTAACCAGTG
GAAACACAAAAATATAAQGGGCTTCTGAGGCGATCGGGCAGTGTCAGTCTTCAGCCGCTAAGCCGAGA
AGATCTGGGAAGGAGTCAGTCAGAGAGCCTTGGGCCAGAGTTCCAGGGGCTCTGGGAGTGGCTGCCAG
AAAATACCAGAAAATGAAAGGAATTGAAATTAAGAGAAGGGAGAGATTGAAGTGTGGCGCCAAGATTG
AAAGGAGAAAGAGGTTGAAGGATAGGGAGGTTGGAGAAGAGAGTAAAAAGAGGCCACTTACTGGATTT
GAAATTGAACCACCCAAAGTCACTGTGATGCCCAAGAATCAGTCTTTCACAGGAGGGTCTGAGGTCTC
CATCATGTGTTCTGCAACAGGTTATCCCAAACCAAAGATTGCCTGGACCGTTAACGATATGTTTATCG
TGGGTTCACACAGGTATAGGATGACCTCAGATGGTACCTTATTTATCAAAAATGCAGCTCCCAAAGAT
GCAGGGATCTATGGTTGCCTAGCAAAAGCCCCTAAGTTGATGGTAGTTCAGAGTGAGCTCTTGGTTGC
CCTTGGGGATATAACCGTTATGGAATGCAAAACCTCTGGTATTCCTCCACCTCAAGTTAAATGGTTCA
AAGGAGATCTTGAGTTGAGGCCCTCAACATTCCTCATTATTGACCCTCTCTTGGGACTTTTGAAGATT
CAAGAAACACAAGATCTGGATGCTGGCGATTATACCTGTGTAGCCATCAATGAGGCTGGAAGAGCAAC
AAATTGGCTCAAATGTGACATTACCTTGTTATGTTCAGGGTTATCCAGAACCAACAATCAAATGGCGA
AGATTAGACAACATGCCAATTTTCTCAAGACCTTTTTCAGTTAGTTCCATCAGCCAACTAAGAACAGG
AGCTCTCTTTATTTTAAACTTATGGGCAAGTGATAAAGGAACCTATATTTGTGAAGCTGAAAACCAGT
TTGGAAAGATCCAGTCAGAGACAACAGTAACAGTGACCGGACTTGTTGCTCCACTTATTGGAATCAGC
CCTTCAGTGGCCAATGTTATTGAAGGACAGCAGCTTACTTTGCCCTGTACTCTGTTAGCTGGAAATCC
CATTCCAGAACGTCGGTGGATTAAGAATTCAGCTATGTTGCTCCAAAATCCTTACATCACTGTGCGCA
GTGATGGGAGCCTCCATATTGAAAGAGTTCAGCTTCAGGATGGTGGTGAATATACTTGTGTGGCCAGT
AACGTTGCTGGGACCAATAACAAAACTACCTCTGTGGTTGTGCATGTTCTGCCAACCATTCAGCATGG
GCAGCAGATACTCAGTACAATTGAAGGCATTCCAGTAACTTTACCATGCAAAGCAAGTGGAAATCCCA
AACCGTCTGTCATCTGGTCCAAGGTAAATGATACATCTAGTTATATTTCCTGAAGAGCAGAGTGTGAA
GTTCACCTGCAAGTTATCCCTAGTCTTGAGCAGGAGGCTCAGGAGTGGGGCATGGAAAGAAGATAAGT
TAATAAAGGATTTCCTATGTGGCTGGACAGATGTGCTAGGAACCCTCCAAGAAACCATATAGATGCAC
CTCAGAAGGCTCCCTCGGCTTTTCGCCGTGTTTTGCAGAAAGGAGAGCTGATTTCAACCAGCAGTGCT
AAGTTTTCAGCAGGAGCTGATGGTAGTCTGTATGTGGTATCACCTGGAGGAGAGGAGAGTGGGGAGTA
TGTCTGCACTGCCACCAATACAGCCGGCTACGCCAAAAGGAAAGTGCAGCTAACAGTCTATGTAAGGC
CCAGAGTGTTTGGAGATCAACGAGGACTGTCCCAGGATAAGCCTGTTGAGATCTCCGTCCTTGCAGGG
GAAGAGGTAACACTTCCATGTGAAGTGAAGAGCTTACCTCCACCCATAATTACTTGGGCCAAAGAAAC
CCAGCTCATCTCACCGTTCTCTCCAAGACACACATTCCTCCCTTCTGGTTCAATGAAGATCACTGAAA
CCCGCACTTCAGATAGTGGGATGTATCTTTGTGTTGCCACAAATATTGCTGGGAATGTGACTCAGGCT
GTCAAATTAAATGTCCATGTTCCTCCAAAGATACAGCGTGGACCTAAACATCTCAAAGTCCAAGTTGG
TCAAAGAGTGGATATTCCATGTAATGCTCAAGGGACTCCTCTTCCTGTAATCACCTGGTCCAAAGGTG
GAAGCACTATGCTGGTTGATGGAGAGCACCATGTTAGCAATCCAGACGGAACTTTAAGCATCGACCAA
GATAACGCTACATGTCCAAGAACCACCCACAGTGGAAGATCTAGAACCTCCATATAACACTACTTTCC
AAGAAAGAGTGGCCAATCAACGCATTGAATTTCCATGTCCTGCAAAAGGTACCCCTAAACCAACCATC
AAATGGTTACACAATGGTAGAGAGTTGACAGGCAGAGAGCCTGGCATTTCTATCTTGGAAGATGGCAC
ATTGCTGGTTATTGCTTCTGTTACACCCTATGACAATGGGGAGTACATCTGTGTGGCAGTCAATGAAG
CTGGAACCACAGAAAGAAAATATAACCTCAAAGTCCATGTTCCTCCAGTAATTAAAGATAAAGAACAA
GTTACAAATGTGTCGGTGTTGTTAAATCAGCTGACCAATCTCTTCTGTGAAGTGGAAGGCACTCCATC
TCCCATCATTATGTGGTATAAAGATAATGTCCAGGTGACTGAAAGCAGCACTATTCAGACTGTGAACA
ATGGGAAGATACTGAAGCTCTTCAGAGCCACTCCAGAGGATGCAGGAAGATATTCCTGCAAAGCAATT
AATATTGCAGGCACTTCTCAGAAGTACTTTAACATTGATGTGCTAGGTACCAACTTCCCAAATGAAGT
CTCAGTTGTCCTCAACCGTGACGTCGCCCTTGAATGCCAGGTCAAAGGCACTCCCTTTCCTGATATTC
ATTGGTTCAAAGATGGCAATATTAAAGGAGGAAATGTCACCACAGACATATCAGTATTGATCAACAGC
CTTATTAAACTGGAATGTGAAACACGGGGACTTCCAATGCCTGCCATTACTTGGTATAAGGACGGGCA
GCCAATCATGTCCAGCTCACAAGCACTTTATATTGATAAAGGACAATATCTTCATATTCCTCGAGCAC
AGGTCTCTGATTCAGCAACATATACGTGTCACGTAGCCAATGTTGCTGGAACTGCTGAAAAATCATTC
CATGTGGATGTCTATGTTCCTCCAATGATTCAAGGCAACTTGGCCACGCCTTTGAATAAGCAAGTAGT
TATTGCTCATTCTCTGACACTGGAGTGCAAAGCTGCTGGAAACCCTTCTCCCATTCTCACCTGGTTGA
AAGATGGTGTACCTGTGAAAGCTAATGACAATATCCGCATAGAAGCTGGTGGGAAGAAACTCGAAATC
ATGAGTGCCCAAGAAATTGATCGAGGACAGTACATATGCGTGGCTACCAGTGTGGCAGGAGAAAAGGA
AATCAAATATGAAGTTGATGTCTTGGTGCCACCAGCTATAGAAGGAGGAGATGAAACATCTTACTTCA
TTGTGATGGTTAATAACTTACTGGAGCTAGATTGTCATGTGACAGGCTCTCCCCCACCAACTATCATG
TGGCTGAAGGATGGCCAGTTAATTGATGAAAGGGATGGATTCAAGATTTTATTAAATGGACGCAAACT
GGTTATTGCTCAGGCTCAAGTGTCAAACACAGGCCTTTATCGGTGCATGGCAGCAAATACTGCTGGAG
ACCACAAGAAGGAATTTGAAGTGACTGTTCATGTTCCTCCAACAATCAAGTCCTCAGGCCTTTCTGAG
AGAGTTGTGGTAAAATACAAGCCTGTCGCCTTGCAGTGCATAGCCAATGGGATTCCAAATCCTTCCAT
TACATGGTTAAAAGATGACCAGCCTGTGAACACTGCCCAAGGAAACCTTAAAATACAGTCTTCTGGTC
GAGTTCTACAAATTGCCAAAACCCTGTTGGAAGATGCTGGCAGATACACATGTGTGGCTACCAACGCA
GCTGGAGAAACACAACAGCACATTCAACTGCATGTTCATGAACCACCTAGTCTGGAAGATGCTGGAAA
AATGCTGAATGAGACTGTGTTGGTGAGCAACCCTGTACAGCTGGAGTGTAAGGCAGCTGGAAATCCTG
TGCCTGTTATTACATGGTACAAAGATAATCGTCTACTCTCAGGTTCCACCAGCATGACTTTCTTGAAC
AGAGGACAGATCATTGATATTGAAAGTGCCCAGATCTCAGATGCTGGCATATATAAATGCGTGGCCAT
CAACTCAGCTGGAGCTACAGAGTTATTTTACAGTCTGCAAGTTCATGTGGCCCCATCAATTTCTGGCA
GCAATAACATGGTGGCAGTGGTGGTTAATAACCCGGTGAGGTTAGAATGTGAAGCCAGAGGTATTCCT
GCCCCAAGTCTGACCTGGTTGAAAGATGGGAGTCCTGTTTCTAGTTTTTCTAATGGATTACAGGTTCT
CTCTGGTGGTCGAATCCTAGCATTGACCAGTGCACAAATCAGCGACACAGGAAGGTACACCTGCGTGG
CAGTGAATGCTGCTGGAGAAAAGCAAAGGGACATTGACCTCCGAGTATATGTTCCGCCAAATATTATG
GGAGAAGAACAGAATGTCTCTGTCCTCATTAGCCAAGCTGTGGAATTACTATGTCAAAGTGATGCTAT
TCCCCCACCTACTCTTACTTGGTTAAAAGACGGCCACCCCTTGCTGAAGAAACCAGGCCTCAGTATAT
CTGAAAATAGAAGTGTGTTAAAGATTGAAGATGCTCAGGTTCAAGACACTGGTCGTTACACTTGTGAA
GCAACAAATGTTGCTGGAAAAACTGAAAAAAACTACAATGTCAACATTTGGGTCCCCCCAAATATTGG
TGGTTCTGATGAACTTACTCAACTTACAGTCATTGAAGGGAATCTCATTAGTCTGTTGTGTGAATCAA
GTGGTATTCCACCCCCAAATCTCATCTGGAAGAAGAAAGGCTCTCCAGTGCTGACTGATTCCATGGGG
CGAGTTAGAATTTTATCTGGGGGCAGGCAATTACAAATTTCAATTGCTGAAAAGTCTGATGCAGCACT
CTATTCATGTGTGGCGTCGAATGTTGCTGGGACTGCAAAGAAAGAATACAATCTGCAAGTTTACATTA
GACCAACCATAACCAACAGTGGCAGCCACCCTACTGAAATTATTGTGACCCGAGGGAAGAGTATCTCC
TTGGAGTGTGAGGTGCAGGGTATTCCACCACCAACAGTGACCTGGATGAAAGATGGCCACCCCTTGAT
CAAGGCAAAGGGAGTAGAAATACTGGATGAAGGTCACATCCTTCAGCTGAAGAACATTCATGTATCTG
ACACAGGCCGTTATGTGTGTGTTGCTGTGAATGTAGCAGGAATGACTGACAAAAAATATGACTTAAGT
GTCCATGGAGGCAGGATGCTACGGCTGATGCAGACCACAATGGAAGATGCTGGCCAATATACTTGCGT
TGTAAGGAATGCAGCTGGTGAAGAAAGAAAAATCTTTGGGCTTTCAGTATTAGTACCACCTCATATTG
TGGGTGAAAATACATTGGAAGATGTGAAGGTAAAAGAGAAACAGAGTGTTACGCTGACTTGTGAAGTG
ACAGGGAATCCAGTGCCAGAAATTACATGGCACAAAGATGGGCAGCCCCTCCAAGAAGATGAAGCCCA
TCACATTATATCTGGTGGCCGTTTTCTTCAAATTACCAATGTCCAGGTGCCACACACTGGAAGATATA
CATGTTTGGCTTCCAGTCCAGCTGGCCACAAGAGCAGGAGCTTCAGTCTTAATGTATTTGTATCTCCT
ACAATTGCTGGTGTAGGTAGTGATGGCAACCCTGAAGATGTCACTGTCATCCTTAACAGCCCTACATC
TTTGGTCTGTGAAGCTTATTCATATCCTCCAGCTACCATCACCTGGTTTAAGGATGGCACTCCTTTAG
AATCTAACCGAAATATTCGTATTCTTCCAGGAGGCAGAACTCTGCAGATCCTCAATGCACAGGAGGAC
AATGCTGGAAGATACTCTTGTGTAGCCACGAATGAGGCTGGAGAAATGATAAAGCACTATGAAGTGAA
GGTGTACACACTTAATGCTAACATTGTTATAATTGAATCACAGCCCCTTAAATCCGATGATCATGTTA
ATATTGCTGCGAATGGACACACACTTCAAATAAAGGAGGCTCAAATATCAGACACCGGACGATATACT
TGTGTAGCATCTAACATTGCAGGTGAAGATGAGTTGGATTTTGATGTGAATATTCAAGTTCCTCCAAG
TTTTCAGAAACTCTGGGAAATAGGAAACATGCTAGATACTGGCAGGAATGGTGAAGCCAAAGATGTGA
TCATCAACAATCCCATTTCTCTTTACTGTGAGACAAATGCTGCTCCCCCTCCTACACTGACATGGTAC
AAAGATGGCCACCCTCTGACCTCAAGTGATAAAGTATTGATTTTGCCAGGAGGGCGAGTGTTGCAGAT
TCCTCGGGCTAAAGTAGAAGATGCTGGGAGATACACATGTGTGGCTGTGAATGAGGCTGGAGAAGATT
CCCTTCAATATGATGTCCGTGTACTCGTGCCGCCAATTATCAAGGGAGCAAATAGTGATCTCCCTGAA
GAGGTCACCGTGCTGGTGAACAAGAGTGCACTGATAGAGTGTTTATCCAGTGGCAGCCCAGCACCAAG
GAATTCCTGGCAGAAAGATGGACAGCCCTTGCTAGAAGATGACCATCATAAATTTCTATCTAATGGAC
GAATTCTGCAGATTCTGAATACTCAAATAACAGATATCGGCAGGTATGTGTGTGTTGCTGAGAACACA
GCTGGGAGTGCCAAAAAATATTTTAACCTCAATGTTCATGTTCCTCCAAGTGTCATTGGTCCTAAATC
TGAAAATCTTACCGTCGTGGTGAACAATTTCATCTCTTTGACCTGTGAGGTCTCTGGTTTTCCACCTC
GGTCGAACTCTACAGATTATTCGGGCCAAGGTATCAGATGGTGGTGAATACACTTGTATAGCTATCAA
TCAAGCTGGCGAAAGCAAGAAAAAGTTTTCCCTGACTGTTTATGTGCCCCCAAGCATTAAAGACCATG
ACAGTGAATCTCTTTCTGTAGTTAATGTAAGAGAGGGAACTTCTGTGTCTTTGGAGTGTGAGTCGAAC
GCTGTGCCACCTCCAGTCATCACTTGGTATAAGAATGGGCGGATGATAACAGAGTCTACTCATGTGGA
GATTTTAGCTGATGGACAAATGCTACACATTAAGAAAGCTGAGGTATCTGACACAGGCCAGTATGTAT
GTAGAGCTATAAATGTAGCAGGACGGGATGATAAAAATTTCCACCTCAATGTATATGTGCCACCCAGT
ATTGAAGGACCTGAAAGAGAAGTGATTGTGGAGACGATCAGCAATCCTGTGACATTAACATGTGATGC
CACTGGGATCCCACCTCCCACGATAGCATGGTTAAAGAACCACAAGCGCATAGAAAATTCTGACTCAC
TGGAAGTTCGTATTTTGTCTGGAGGTAGCAAACTCCAGATTGCCCGGTCTCAGCATTCAGATAGTGGA
AACTATACATGTATTGCTTCAAATATGGAGGGAAAAGCCCAGAAATATTACTTTCTTTCAATTCAAGT
TCCTCCAAGTGTTGCTGGTGCTGAAATTCCAAGTGATGTCAGTGTCCTTCTAGGAGAAAATGTTGAGC
TGGTCTGCAATGCAAATGGCATTCCTACTCCACTTATTCAATGGCTTAAAGATGGAAAGCCCATAGCT
AGTGGTGAAACAGAAAGAATCCGAGTGAGTGCAAATGGCAGCACATTAAACATTTATGGAGCTCTTAC
ATCTGACACGGGGAAATACACATGTGTTGCTACTAATCCCGCTGGAGAAGAAGACCGAATTTTTAACT
TGAATGTCTATGTTACACCTACAATTAGGGGTAATAAAGATGAAGCAGAGAAACTAATGACTTTAGTG
GATACTTCAATAAATATTGAATGCAGAGCCACAGGGACGCCTCCACCACAGATAAACTGGCTGAAGAA
TGGACTTCCTCTGCCTCTCTCCTCCCATATCCGGTTACTGGCAGCAGGACAAGTTATCAGGATTGTGA
GAGCTCAGGTGTCTGATGTCGCTGTGTATACTTGTGTGGCCTCCAACAGAGCTGGGGTGGATAATAAG
CATTACAATCTTCAAGTGTTTGCACCACCAAATATGGACAATTCAATGGGGACAGAGGAAATCACAGT
TCTCAAAGGTAGTTCCACCTCTATGGCATGCATTACTGATGGAACCCCAGCTCCCAGTATGGCCTGGC
TTAGAGATGGCCAGCCTCTGGGGCTTGATGCCCATCTGACAGTCAGCACCCATGGAATGGTCCTGCAG
CTCCTCAAAGCAGAGACTGAAGATTCGGGAAAGTACACCTGCATTGCCTCAAATGAAGCTGGAGAAGT
CAGCAAGCACTTTATCCTCAAGGTCCTAGAACCACCTCACATTAATGGATCTGAAGAACATGAAGAGA
TATCAGTAATTGTTAATAACCCACTTGAACTTACCTGCATTGCTTCTGGAATCCCAGCCCCTAAAATG
ACCTGGATGAAAGATGGCCGGCCCCTTCCACAGACGGATCAAGTGCAAACTCTAGGAGGAGGAGAGGT
TCTTCGAATTTCTACTGCTCAGGTGGAGGATACAGGAAGATATACATGTCTGGCATCCAGTCCTGCAG
GAGATGATGATAAGGAATATCTAGTGAGAGTGCATGTACCTCCTAATATTGCTGGAACTGATGAGCCC
CGGGATATCACTGTGTTACGGAACAGACAAGTGACATTGGAATGCAAGTCAGATGCAGTGCCCCCACC
TGTAATTACTTGGCTCAGAAATGGAGAACGGTTACAGGCAACACCTCGAGTGCGAATCCTATCTGGAG
GGAGATACTTGCAAATCAACAATGCTGACCTAGGTGATACAGCCAATTATACCTGTGTTGCCAGCAAC
ATTGCAGGAAAGACTACAAGAGAATTTATTCTCACTGTAAATGTTCCTCCAAACATAAAGGGGGGCCC
CCAGAGCCTTGTAATTCTTTTAAATAAGTCAACTGTATTGGAATGCATCGCTGAAGGTGTCCCAACTC
CAAGGATAACATGGAGAAAGGATGGAGCTGTTCTAGCTGGGAATCATGCAAGATATTCCATCTTGGAA
AATGGATTCCTTCATATTCAATCAGCACATGTCACTGACACTGGACGGTATTTGTGTATGGCCACCAA
TGCTGCTGGAACAGATCGCAGGCGAATAGATTTACAGGTCCATGTTCCTCCATCTATTGCTCCGGGTC
CTACCAACATGACTGTAATAGTAAATGTTCAAACTACTCTGGCTTGTGAGGCTACTGGGATACCAAAA
CCATCAATCAATTGGAGAAAAAATGGGCATCTTCTTAATGTGGATCAAAATCAGAACTCATACAGGCT
CCTTTCTTCAGGTTCACTAGTAATTATTTCCCCTTCTGTGGATGACACTGCAACCTATGAATGTACTG
TGACAAACGGTGCTGGAGATGATAAAAGAACTGTGGATCTCACTGTCCAAGTTCCACCTTCCATAGCT
GATGAGCCTACAGATTTCCTAGTAACCAAACATGCCCCAGCAGTAATTACCTGCACTGCTTCGGGAGT
TTCTGTCCTCAGGAGCAATTGAAATACTTGCCACCCAATTAAACCATGCTGGAAGATACACTTGTGTC
GCTAGGAATGCGGCTGGCTCTGCACATCGACACGTGACCCTTCATGTTCATGAGCCTCCAGTCATTCA
GCCCCAACCAAGTGAACTACACGTCATTCTGAACAATCCTATTTTATTACCATGTGAAGCAACAGGGA
CACCCAGTCCTTTCATTACTTGGCAAAAAGAAGGCATCAATGTTAACACTTCAGGCAGAAACCATGCA
GTTCTTCCTAGTGGCGGCTTACAGATCTCCAGAGCTGTCCGAGAGGATGCTGGCACTTACATGTGTGT
GGCCCAGAACCCGGCTGGTACAGCCTTGGGCAAAATCAAGTTAAATGTCCAAGTTCCTCCAGTCATTA
GCCCTCATCTAAAGGAATATGTTATTGCTGTGGACAAGCCCATCACGTTATCCTGTGAAGCAGATGGC
CTCCCTCCGCCTGACATTACATGGCATAAAGATGGGCGTGCAATTGTGGAATCTATCCGCCAGCGCGT
CCTCAGCTCTGGCTCTCTGCAAATAACATTTGTCCAGCCTGGTGATGCTGGCCATTACACGTGCATGG
CAGCCAATGTAGCAGGATCAAGCAGCACAAGCACCAAGCTCACCGTCCATGTACCACCCAGGATCAGA
AGTACAGAAGGACACTACACGGTCAATGAGAATTCACAAGCCATTCTTCCATGCGTAGCTGATGGAAT
CCCCACACCAGCAATTAACTGGAAAAAAGACAATGTTCTTTTAGCTAACTTGTTAGGAAAATACACTG
CTGAACCATATGGAGAACTCATTTTAGAAAATGTTGTGCTGGAGGATTCTGGCTTCTATACCTGTGTT
GCTAACAATGCTGCAGGTGAAGATACACACACTGTCAGCCTGACTGTGCATGTTCTCCCCACTTTTAC
TGAACTTCCTGGAGACGTGTCATTAAATAAAGGAGAACAGCTACGATTAAGCTGTAAAGCTACTGGTA
TTCCATTGCCCAAATTAACATGGACCTTCAATAACAATATTATTCCAGCCCACTTTGACAGTGTGAAT
GGACACAGTGAACTTGTTATTGAAAGAGTGTCAAAAGAGGATTCAGGTACTTATGTGTGCACCGCAGA
GAACAGCGTTGGCTTTGTGAAGGCAATTGGATTTGTGTATGTGAAAGAACCTCCAGTCTTCAAAGGTG
ATTATCCTTCTCACTGGATTGAACCACTTGGTGGGAATGCAATCCTGAATTGTGAGGTGAAAGGAGAC
CCCACCCCAACCATCCAGTGGAACAGAAAGGGAGTGGATATTGAAATTAGCCACAGAATCCGGCAACT
GGGCAATGGCTCCCTGGCCATCTATGGCACTGTTAATGAAGATGCCGGTGACTATACATGTGTAGCTA
CCAATGAAGCTGGGGTGGTGGAGCGCAGCATGAGTCTGACTCTGCAAAGTCCTCCTATTATCACTCTT
GAGCCAGTGGAAACTGTTATTAATGCTGGTGGCAAAATCATATTGAATTGTCAGGCAACTGGAGAGCC
TCAACCAACCATTACATGGTCCCGTCAAGGGCACTCTATTTCCTGGGATGACCGGGTTAACGTGTTGT
CCAACAACTCATTATATATTGCTGATGCTCAGAAAGAAGATACCTCTGAATTTGAATGTGTTGCTCGA
AACTTAATGGGTTCTGTCCTTGTCAGAGTGCCAGTCATAGTCCAGGTTCATGGTGGATTTTCCCAGTG
GTCTGCATGGAGAGCCTGCAGTGTCACCTGTGGAAAAGGCATCCAAAAGAGGAGTCGTCTGTGCAACC
AGCCCCTTCCAGCCAATGGTGGGAAGCCCTGCCAAGGTTCAGATTTGGAAATGCGAAACTGTCAAAAT
AAGCCTTGTCCAGTGGATGGTAGCTGGTCGGAATGGAGTCTTTGGGAAGAATGCACAAGGAGCTGTGG
ACGCGGCAACCAAACCAGGACCAGGACTTGCAATAATCCATCAGTTCAGCATGGTGGGCGGCCATGTG
AAGGGAATGCTGTGGAAATAATTATGTGCAACATTAGGCCTTGCCCAGTTCATGGAGCATGGAGCGCT
TGGCAGCCTTGGGGAACATGCAGCGAAAGTTGTGGGAAAGGTACTCAGACAAGAGCAAGACTTTGTAA
AAAGAAATTGTCCAGTTCATGGCAAGTGGGCGACTTGGGCCAGTTGGAGTGCCTGTTCTGTGTCATGT
GGAGGAGGTGCCAGACAGAGAACAAGGGGCTGCTCCGACCCTGTGCCCCAGTATGGAGGAAGGAAATG
CGAAGGGAGTGATGTCCAGAGTGATTTTTGCAACAGTGACCCTTGCCCAACCCATGGTAACTGGAGTC
CTTGGAGTGGCTGGGGAACATGCAGCCGGACGTGTAACGGAGGGCAGATGCGGCGGTACCGCACATGT
GATAACCCTCCTCCCTCCAATGGGGGAAGAGCTTGTGGGGGACCAGACTCCCAGATCCAGAGGTGCAA
CACTGACATGTGTCCTGTGGATGGAAGTTGGGGAAGCTGGCATAGTTGGAGCCAGTGCTCTGCCTCCT
GTGGAGGAGGTGAAAAGACTCGGAAGCGGCTGTGCGACCATCCTGTGCCAGTTAAAGGTGGCCGTCCC
TGTCCCGGAGACACTACTCAGGTGACCAGGTGCAATGTACAAGCATGTCCAGGTGGGCCCCAGCGAGC
CAGAGGAAGTGTTATTGGAAATATTAATGATGTTGAATTTGGAATTGCTTTCCTTAATGCCACAATAA
CTGATAGCCCTAACTCTGATACTAGAATAATACGTGCCAAAATTACCAATGTACCTCGTAGTCTTGGT
TCAGCAATGAGAAAGATAGTTTCTATTCTAAATCCCATTTATTGGACAACAGCAAAGGAAATAGGAGA
AGCAGTCAATGGCTTTACCCTCACCAATGCAGTCTTCAAAAGAGAAACTCAAGTGGAATTTGCAACTG
GAGAAATCTTGCAGATGAGTCATATTGCCCGGGGCTTGGATTCCGATGGTTCTTTGCTGCTAGATATC
GTTGTGAGTGGCTATGTCCTACAGCTTCAGTCACCTGCTGAAGTCACTGTAAAGGATTACACAGAGGA
CTACATTCAAACAGGTCCTGGGCAGCTGTACGCCTACTCAACCCGGCTGTTCACCATTGATGGCATCA
GCATCCCATACACATGGAACCACACCGTTTTCTATGATCAGGCACAGGGAAGAATGCCTTTCTTGGTT
GAAACACTTCATGCATCCTCTGTGGAATCTGACTATAACCAGATAGAAGAGACACTGGGTTTTAAAAT
TCATGCTTCAATATCCAAAGGAGATCGCAGTAATCAGTGCCCCTCCGGGTTTACCTTAGACTCAGTTG
GACCTTTTTGTGCTGATGAGGATGAATGTGCAGCAGGGAATCCCTGCTCCCATAGCTGCCACAATGCC
ATGGGGACTTACTACTGCTCCTGCCCTAAAGGCCTCACCATAGCTGCAGATGGAAGAACTTGTCAAGA
TATTGATGAGTGTGCTTTGGGTAGGCATACCTGCCACGCTGGTCAGGACTGTGACAATACGATTGGAT
CTTATCGCTGTGTGGTCCGTTGTGGAAGTGGCTTTCGAAGAACCTCTGATGGGCTGAGTTGTCAAGAT
ATTAATGAATGTCAAGAATCCAGCCCCTGTCACCAGCGCTGTTTCAATGCCATAGGAAGTTTCCATTG
TGGATGTGAACCTGGGTATCAGCTCAAAGGCAGAAAATGCATGGATGTGAACGAGTGTAGACAAAATG
TATGCAGACCAGATCAGCACTGTAAGAACACCCGTGGTGGCTATAAGTGCATTGATCTTTGTCCAAAT
GGAATGACCAAGGCAGAAAATGGAACCTGTATTGATATTGATGAATGTAAAGATGGGACCCATCAGTG
CAGATATAACCAGATATGTGAGAATACAAGAGGCAGCTATCGTTGTGTATGCCCAAGAGGTTATCGGT
CTCAAGGAGTTGGAAGACCCTGCATGGACATTAATGAATGTGAACAAGTGCCTAAACCTTGTGCACAT
CAGTGCTCCAACACCCCCGGCAGCTTCAAGTGTATCTGTCCACCAGGACAACATTTATTAGGGGACGG
GAAATCTTGCGCTGGATTGGAGAGGCTGCCAAATTATGGCACTCAATACAGTAGCTATAACCTTGCAC
GGTTCTCCCCTGTGAGAAACAACTATCAACCTCAACAGCATTACAGACAGTACTCACATCTCTACAGC
TCCTACTCAGAGTATAGAAACAGCAGAACATCTCTCTCCAGGACTAGAAGGACTATTAGGAAAACTTG
CCCTGAAGGCTCTGAGGCAAGCCATGACACATGTGTAGATATTGATGAATGTGAAAATACAGATGCCT
GCCAGCATGAGTGTAAGAATACCTTTGGAAGTTATCAGTGCATCTGCCCACCTGGCTATCAACTCACA
CACAATGGAAAGACATGCCAAGATATCGATGAATGTCTGGAGCAGAATGTGCACTGTGGACCCAATCG
CATGTGCTTCAACATGAGAGGAAGCTACCAGTGCATCGATACACCCTGTCCACCCAACTACCAACGGG
ATCCTGTTTCAGGGTTCTGCCTCAAGAACTGTCCACCCAATGATTTGGAATGTGCCTTGAGCCCATAT
GCCTTGGAATACAAACTCGTCTCCCTCCCATTTGGAATAGCCACCAATCAAGATTTAATCCGGCTGGT
TGCATACACACAGGATGGAGTGATGCATCCCAGGACAACTTTCCTCATGGTAGATGAGGAACAGACTG
TTCCTTTTGCCTTGAGGGATGAAAACCTGAAAGGAGTGGTGTATACAACACGACCACTACGAGAAGCA
GAGACCTACCGCATGAGGGTCCGAGCCTCATCCTACAGTGCCAATGGGACCATTGAATATCAGACCAC
ATTCATAGTTTATATAGCTGTGTCCGCCTATCCATACTAAGGAACTCTCCAAAGCCTATTCCACATAT
TTAAACCGCATTAATCATGGCAATCAAGCCCCCTTCCAGATTACTGTCTCTTGAACAGTTGCAATCTT
GGCAGCTTGAAAATGGTGCTACACTCTGTTTTGTGTGCCTTCCTTGGTACTTCTGAGGTATTTTCATG
ATCCCACCATGGTCATATCTTGAAGTATGGTCTAGAAAAGTCCCTTATTATTTTATTTATTACACTGC
AGCACTTACTTCCCAAAGATTATTCTGAACATCTAACAGGACATATCAGTGATGGTTTACAGTAGTGT
AGTACCTAAGATCATTTTCCTGAAAGCCAAACCAAACAACGAAAAACAAGAACAACTAATTCAGAATC
AAATAGAGTTTTTGAGCATTTGACTATTTTTAGAATCATAAAATTAGTTACTAAGTATTTTGATCAAA
GCTTATAAAATAACTTACGGAGATTTTTGTAAGTATTGATACATTATAATAGGACTTGCCTATTTTCA
TTTTTAAGAAGAAAAACCCG

| | | | |
|---|---|---|---|
| NOV43c, CG56914-02 | SEQ ID NO:1002 | 4495 aa | MW at 488830.5 kD |
| Protein Sequence | | | |

MWLDRCARNPPRNHIDAPQKAPSAFRRVLQKGELISTSSAKFSAGADGSLYVVSPGGEESGEYVCTAT
NTAGYAKRKVQLTVYVRPRVFGDQRGLSQDKPVEISVLAGEEVTLPCEVKSLPPPTITWAKETQLISP
FSPRHTFLPSGSMKITETRTSDSGMYLCVATNIAGNVTQAVKLNVHVPPKIQRGPKHLKVQVGQRVDI
PCNAQGTPLPVITWSKGGSTMLVDGEHHVSNPDGTLSIDQATPSDAGIYTCVATNIAGTDETEITLHV
QEPPTVEDLEPPYNTTFQERVANQRIEFPCPAKGTPKPTIKWLHNGRELTGREPGISILEDGTLLVIA
SVTPYDNGEYICVAVNEAGTTERKYNLKVHVPPVIKDKEQVTNVSVLLNQLTNLFCEVEGTPSPIIMW
YKDNVQVTESSTIQTVNNGKILKLFRATPEDAGRYSCKAINIAGTSQKYFNIDVLGTNFPNEVSVVLN
RDVALECQVKGTPFPDIHWFKDGNIKGGNVTTDISVLINSLIKLECETRGLPMPAITWYKDGQPIMSS
SQALYIDKGQYLHIPRAQVSDSATYTCHVANVAGTAEKSFHVDVYVPPMIEGNLATPLMKQVVIAHSL
TLECKAAGNPSPILTWLKDGVPVKANDNIRIEAGGKKLEIMSAQEIDRGQYICVATSVAGEKEIKYEV
DVLVPPAIEGGDETSYFIVMVNNLLELDCHVTGSPPPTIMWLKDGQLIDERDGFKILLNGRKLVIAQA
QVSNTGLYRCMAANTAGDHKKEFEVTVHVPPTIKSSGLSERVVVKYKPVALQCIANGIPNPSITWLKD
DQPVNTAQGNLKIQSSGRVLQIAKTLLEDAGRYTCVATNAAGETQQHIQLHVHEPPSLEDAGKMLNET
VLVSNPVQLECKAAGNPVPVITWYKDNRLLSGSTSMTFLNRGQIIDIESAQISDAGIYKCVAINSAGA
TELFYSLQVHVAPSISGSNNMVAVVVNNPVRLECEARGIPAPSLTWLKDGSPVSSFSNGLQVLSGGRI
LALTSAQISDTGRYTCVAVNAAGEKQRDIDLRVYVPPNIMGEEQNVSVLISQAVELLCQSDAIPPPTL
TWLKDGHPLLKKPGLSISENRSVLKIEDAQVQDTGRYTCEATNVAGKTEKNYNVNIWVPPNIGGSDEL
TQLTVIEGNLISLLCESSGIPPPNLIWKKKGSPVLTDSMGRVRTLSGGRQLQISIAEKSDAALYSCVA
SNVAGTAKKEYNLQVYIRPTITNSGSHPTEIIVTRGKSISLECEVQGIPPPTVTWMKDGHPLIKAKGV
EILDEGHILQLKNIHVSDTGRYVCVAVNVAGMTDKKYDLSVHGGRMLRLMQTTMEDAGQYTCVVRNAA
GEERKIFGLSVLVPPHIVGENTLEDVKVKEKQSVTLTCEVTGNPVPEITWHKDGQPLQEDEAHHIISG
GRFLQITNVQVPHTGRYTCLASSPAGHKSRSFSLNVFVSPTIAGVGSDGNPEDVTVILNSPTSLVCEA
YSYPPATITWFKDGTPLESNPNIRILPGGRTLQILNAQEDNAGRYSCVATNEAGEMIKHYEVKVYTLN
ANIVIIESQPLKSDDHVNIAANGHTLQIKEAQISDTGRYTCVASNIAGEDELDFDVNIQVPPSFQKLW
EIGNMLDTGRNGEAKDVIINNPISLYCETNAAPPPTLTWYKDGHPLTSSDKVLILPGGRVLQIPRAKV
EDAGRYTCVAVNEAGEDSLQYDVRVLVPPIIKGANSDLPEEVTVLVNKSALIECLSSGSPAPRNSWQK
DGQPLLEDDHHKFLSNGRILQILNTQITDIGRYVCVAENTAGSAKKYFNLNVHVPPSVIGPKSENLTV
VVNNFISLTCEVSGFPPPDLSWLKNEQPIKLNTNTLIVPGGRTLQIIRAKVSDGGEYTCIAINQAGES
KKKFSLTVYVPPSIKDHDSESLSVVNVREGTSVSLECESNAVPPPVITWYKNGRMITESTHVEILANG
QMLHIKKAEVSDTGQYVCRAINVAGRDDKNFHLNVYVPPSIEGPEREVIVETISNPVTLTCDATGIPP
PTIAWLKNHKRIENSDSLEVRILSGGSKLQIARSQHSDSGNYTCIASNMEGKAQKYYFLSIQVPPSVA
GAEIPSDVSVLLGENVELVCNANGIPTPLIQWLKDGKPIASGETERIRVSANGSTLNIYGALTSDTGK
YTCVATNPAGEEDRIFNLNVYVTPTIRGNKDEAEKLMTLVDTSINIECRATGTPPPQINWLKNGLPLP
LSSHIRLLAAGQVIRIVRAQVSDVAVYTCVASNRAGVDNKHYNLQVFAPPNMDNSMGTEEITVLKGSS
TSMACITDGTPAPSMAWLRDGQPLGLDAHLTVSTHGMVLQLLKAETEDSGKYTCIASNEAGEVSKHFI
LKVLEPPHINGSEEHEEISVIVNNPLELTCIASGIPAPKMTWMKDGRPLPQTDQVQTLGGGEVLRIST
AQVEDTGRYTCLASSPAGDDDKEYLVRVHVPPNIAGTDEPRDITVLRNRQVTLECKSDAVPPPVITWL
RNGERLQATPRVRILSGGRYLQINNADLGDTANYTCVASNIAGKTTREFILTVNVPPNIKGGPQSLVI
LLNKSTVLECIAEGVPTPRITWRKDGAVLAGNHARYSILENGFLHIQSAHVTDTGRYLCMATNAAGTD
RRRIDLQVHVPPSIAPGPTNMTVIVNVQTTLACEATGIPKPSINWRKNGHLLNVDQNQNSYRLLSSGS
LVIISPSVDDTATYECTVTNGAGDDKRTVDLTVQVPPSIADEPTDFLVTKHAPAVITCTASGVPFPSI
HWTKNGIRLLPRGDGYRILSSGAIEILATQLNHAGRYTCVARNAAGSAHRHVTLHVHEPPVIQPQPSE
LHVILNNPILLPCEATGTPSPFITWQKEGINVNTSGRNHAVLPSGGLQISRAVREDAGTYMCVAQNPA
GTALGKIKLNVQVPPVISPHLKEYVIAVDKPITLSCEADGLPPPDITWHKDGRAIVESIRQRVLSSGS
LQITFVQPGDAGHYTCMAANVAGSSSTSTKLTVHVPPRIRSTEGHYTVNENSQAILPCVADGIPTPAI
NWKKDNVLLANLLGKYTAEPYGELILENVVLEDSGFYTCVANNAAGEDTHTVSLTVHVLPTFTELPGD
VSLNKGEQLRLSCKATGIPLPKLTWTFNNNIIPAHFDSVNGHSELVIERVSKEDSGTYVCTAENSVGF
VKAIGFVYVKEPPVFKGDYPSHWIEPLGGNAILNCEVKGDPTPTIQWNRKGVDIEISHRIRQLGNGSL
AIYGTVNEDAGDYTCVATNEAGVVERSMSLTLQSPPIITLEPVETVINAGGKIILNCQATGEPQPTIT
WSRQGHSISWDDRVNVLSNNSLYIADAQKEDTSEFECVARNLMGSVLVRVPVIVQVHGGFSQWSAWRA
CSVTCGKGIQKRSRLCNQPLPANGGKPCQGSDLEMENCQNKPCPVDGSWSEWSLWEECTRSCGRGNQT
RTRTCNNPSVQHGGRPCEGNAVEIIMCNIRPCPVHGAWSAWQPWGTCSESCGKGTQTRAELCNNPPPA
FGGSYCDGAETQMQVCNERNCPVHGKWATWASWSACSVSCGGGARQRTRGCSDPVPQYGGRKCEGSDV
QSDFCNSDPCPTHGNWSPWSGWGTCSRTCNGGQMRRYRTCDNPPPSNGGRACGGPDSQIQRCNTDMCP
VDGSWGSWHSWSQCSASCGGGEKTRKRLCDHPVPVKGGRPCPGDTTQVTRCNVQACPGGPQRARGSVI
GNINDVEFGIAFLNATITDSPNSDTRIIRAKITNVPRSLGSAMRKIVSILNPIYWTTAKEIGEAVNGF
TLTNAVFKRETQVEFATGEILQMSHIARGLDSDGSLLLDIVVSGYVLQLQSPAEVTVKDYTEDYIQTG
PGQLYAYSTRLFTIDGISIPYTWNHTVFYDQAQGRMPFLVETLHASSVESDYNQIEETLGFKIHASIS
KGDRSNQCPSGFTLDSVGPFCADEDECAAGNPCSHSCHNAMGTYYCSCPKGLTIAADGRTCQDIDECA
LGRHTCHAGQDCDNTIGSYRCVVRCGSGFRRTSDGLSCQDINECQESSPCHQRCFNAIGSFHCGCEPG
YQLKGRKCMDVNECRQNVCRPDQHCKNTRGGYKCIDLCPNGMTKAENGTCIDIDECKDGTHQCRYNQI
CENTRGSYRCVCPRGYRSQGVGRPCMDINECEQVPKPCAHQCSNTPGSFKCICPPGQHLLGDGKSCAG
LERLPNYGTQYSSYNLARFSPVRNNYQPQQHYRQYSHLYSSYSEYRNSRTSLSRTRRTIRKTCPEGSE
ASHDTCVDIDECENTDACQHECKNTFGSYQCICPPGYQLTHNGKTCQDIDECLEQNVHCGPNRMCFNM
RGSYQCIDTPCPPNYQRDPVSGFCLKNCPPNDLECALSPYALEYKLVSLPFGIATNQDLIRLVAYTQD
GVMHPRTTFLMVDEEQTVPFALRDENLKGVVYTTRPLREAETYRMRVRASSYSANGTIEYQTTFIVYI
AVSAYPY

| | | |
|---|---|---|
| NOV43d, CG56914-03 | SEQ ID NO:1003 | 6343 bp |
| DNA Sequence | ORF Start: ATG at 105 | ORF Stop: TAA at 5811 |

AACCACCTCACATTAATGGATCTGAAGAACATGAAGAGATATCAGTAATTGTTAATAACCCACTTGAA
CTTACCTGCATTGCTTCTGGAATCCCAGCCCCTAAAATGACCTGGATGAAAGATGGCCGGCCCCTTCC
ACAGACGGATCAAGTGCAAACTCTAGGAGGAGGAGAGGTTCTTCGAATTTCTACTGCTCAGGTGGAGG
ATACAGGAAGATATACATGTCTGGCATCCAGTCCTGCAGGAGATGATGATAAGGAATATCTAGTGAGA
GTGCATGTACCTCCTAATATTGCTGGAACTGATGAGCCCCGGGATATCACTGTGTTACGGAACAGACA
AGTGACATTGGAATGCAAGTCAGATGCAGTGCCCCCACCTGTAATTACTTGGCTCAGAAATGGAGAAC
GGTTACAGGCAACACCTCGAGTGCGAATCCTATCTGGAGGGAGATACTTGCAAATCAACAATGCTGAC
CTAGGTGATACAGCCAATTATACCTGTGTTGCCAGCAACATTGCAGGAAAGACTACAAGAGAATTTAT
TCTCACTGTAAATGTTCCTCCAAACATAAAGGGGGGCCCCCAGAGCCTTGTAATTCTTTTAAATAAGT
CAACTGTATTGGAATGCATCGCTGAAGGTGTGCCAACTCCAAGGATAACATGGAGAAAGGATGGAGCT
GTTCTAGCTGGGAATCATGCAAGATATTCCATCTTGGAAAATGGATTCCTTCATATTCAATCAGCACA
TGTCACTGACACTGGACGGTATTTGTGTATGGCCACCAATGCTGCTGGAACAGATCGCAGGCGAATAG
ATTTACAGGTCCATGGTTCACTAGTAATTATTTCCCCTTCTGTGGATGACACTGCAACCTATGAATGT
ACTGTGACAAACGGTGCTGGAGATGATAAAAGAACTGTGGATCTCACTGTCCAAGTTCCACCTTCCAT
AGCTGATGAGCCTACAGATTTCCTAGTAACCAAACATGCCCCAGCAGTAATTACCTGCACTGCTTCGG
GAGTTCCATTTCCCTCAATTCACTGGACCAAAAATGGTATAAGACTGCTTCCCAGGGGAGATGGCTAT
AGAATTCTGTCCTCAGGAGCAATTGAAATACTTGCCACCCAATTAAACCATGCTGGAAGATACACTTG
TGTCGCTAGGAATGCGGCTGGCTCTGCACATCGACACGTGACCCTTCATGTTCATGAGCCTCCAGTCA
TTCAGCCCCAACCAAGTGAACTACACGTCATTCTGAACAATCCTATTTTATTACCATGTGAAGCAACA
GGGACACCCAGTCCTTTCATTACTTGGCAAAAAGAAGGCATCAATGTTAACACTTCAGGCAGAAACCA
TGCAGTTCTTCCTAGTGGCGGCTTACAGATCTCCAGAGCTGTCCGAGAGGATGCTGGCACTTACATGT
GTGTGGCCCAGAACCCGGCTGGTACAGCCTTGGGCAAAATCAAGTTAAATGTCCAAGTTCCTCCAGTC
ATTAGCCCTCATCTAAAGGAATATGTTATTGCTGTGGACAAGCCCATCACGTTATCCTGTGAAGCAGA
TGGCCTCCCTCCGCCTGACATTACATGGCATAAAGATGGGCGTGCAATTGTGGAATCTATCCGCCAGC
GCGTCCTCAGCTCTGGCTCTCTGCAAATAGCATTTGTCCAGCCTGGTGATGCTGGCCATTACACGTGC
ATGGCAGCCAATGTAGCAGGATCAAGCAGCACAAGCACCAAGCTCACCGTCCATGTACCACCCAGGAT
CAGAAGTACAGAAGGACACTACACGGTCAATGAGAATTCACAAGCCATTCTTCCATGCGTAGCTGATG
GAATCCCCACACCAGCAATTAACTGGAAAAAAGACAATGTTCTTTTAGCTAACTTGTTAGGAAAATAC
ACTGCTGAACCATATGGAGAACTCATTTTAGAAAATGTTGTGCTGGAGGATTCTGGCTTCTATACCTG
TGTTGCTAACAATGCTGCAGGTGAAGATACACACACTGTCAGCCTGACTGTGCATGTTCTCCCCACTT
TTACTGAACTTCCTGGAGACGTGTCATTAAATAAAGGAGAACAGCTACGATTAAGCTGTAAAGCTACT
GGTATTCCATTGCCCAAATTAACATGGACCTTCAATAACAATATTATTCCAGCCCACTTTGACAGTGT
GAATGGACACAGTGAACTTGTTATTGAAAGAGTGTCAAAAGAGGATTCAGGTACTTATGTGTGCACCG
CAGAGAACAGCGTTGGCTTTGTGAAGGCAATTGGATTTGTTTATGTGAAAGAACCTCCAGTCTTCAAA
GGTGATTATCCTTCTAACTGGATTGAACCACTTGGTGGGAATGCAATCCTGAATTGTGAGGTGAAAGG
AGACCCCACCCCAACCATCCAGTGGAACAGAAAGGGAGTGGATATTGAAATTAGCCACAGAATCCGGC
AACTGGGCAATGGCTCCCTGGCCATCTATGGCACTGTTAATGAAGATGCCGGTGACTATACATGTGTA
GCTACCAATGAAGCTGGGGTGGTGGAGCGCAGCATGAGTCTGACTCTGCAAAGTCCTCCTATTATCAC
TCTTGAGCCAGTGGAAACTGTTATTAATGCTGGTGGCAAAATCATATTGAATTGTCAGGCAACTGGAG
AGCCTCAACCAACCATTACATGGTCCCGTCAAGGGCACTCTATTTCCTGGGATGACCGGGTTAACGTG
TTGTCCAACAACTCATTATATATTGCTGATGCTCAGAAAGAAGATACCTCTGAATTTGAATGCGTTGC
TCGAAACTTAATGGGTTCTGTCCTTGTCAGAGTGCCAGTCATAGTCCAGGTTCATGGTGGATTTTCCC
AGTGGTCTGCATGGAGAGCCTGCAGTGTCACCTGTGGAAAAGGCATCCAAAAGAGGAGTCGTCTGTGC
AACCAGCCCCTTCCAGCCAATGGTGGGAAGCCCTGCCAAGGTTCAGATTTGGAAATGCGAAACTGTCA
AAATAAGCCTTGTCCAGTGGATGGTAGCTGGTCGGAATGGAGTCTTTGGGAAGAATGCACAAGGAGCT
GTGGACGCGGCAACCAAACCAGGACCAGGACTTGCAATAATCCATCAGTTCAGCATGGTGGGCGGCCA
TGTGAAGGGAATGCTGTGGAAATAATTATGTGCAACATTAGGCCTTGCCCAGTTCATGGAGCATGGAG
CGCTTGGCAGCCTTGGGGAACATGCAGCGAAAGTTGTGGGAAAGGTACTCAGACAAGAGCAAGACTTT
GTAATAACCCACCACCAGCGTTTGGTGGGTCCTACTGTGATGGAGCAGAAACACAGATGCAAGTTTGC
AATGAAAGAAATTGTCCAATTCATGGCAAGTGGGCGACTTGGGCCAGTTGGAGTGCCTGTTCTGTGTC
ATGTGGAGGAGGTGCCAGACAGAGAACAAGGGGCTGCTCCGACCCTGTGCCCCAGTATGGAGGAAGGA
AATGCGAAGGGAGTGATGTCCAGAGTGATTTTTGCAACAGTGACCCTTGCCCAACCCATGGTAACTGG
AGTCCTTGGAGTGGCTGGGGAACATGCAGCCGGACGTGTAACGGAGGGCAGATGCGGCGGTACCGCAC
ATGTGATAACCCTCCTCCCTCCAATGGGGGAAGAGCTTGTGGGGGACCAGACTCCCAGATCCAGAGGT
GCAACACTGACATGTGTCCTGTGGATGGAAGTTGGGGAAGCTGGCATAGTTGGAGCCAGTGCTCTGCC
TCCTGTGGAGGAGGTGAAAAGACTCGGAAGCGGCTGTGCGACCATCCTGTGCCAGTTAAAGGTGGCCG
TCCTTGTCCCGGAGACACTACTCAGGTGACCAGGTGCAATGTACAAGCATGTCCAGGTGGGCCCCAGC
GAGCCAGAGGAAGTGTTATTGGAAATATTAATGATGTTGAATTTGGAATTGCTTTCCTTAATGCCACA
ATAACTGATAGCCCTAACTCTGATACTAGAATAATACGTGCCAAAATTACCAATGTACCTCGTAGTCT
TGGTTCAGCAATGAGAAAGATAGTTTCTATTCTAAATCCCATTTATTGGACAACAGCAAAGGAAATAG
GAGAAGCAGTCAATGGCTTTACCCTCACCAATGCAGTCTTCAAAAGAGAAACTCAAGTGGAATTTGCA
ACTGGAGAAATCTTGCAGATGAGTCATATTGCCCGGGGCTTGGATTCCGATGGTTCTTTGCTGCTAGA
TATCGTTGTGAGTGGCTATGTCCTACAGCTTCAGTCACCTGCTGAAGTCACTGTAAAGGATTACACAG
AGGACTACATTCAAACAGGTCCTGGGCAGCTGTACGCCTACTCAACCCGGCTGTTCACCATTGATGGC
ATCAGCATCCCATACACATGGAACCACACCGTTTTCTATGATCAGGCACAGGGAAGAATGCCTTTCTT
GGTTGAAACACTTCATGCATCCTCTGTGGAATCTGACTATAACCAGATAGAAGAGACACTGGGTTTTA
AAATTCATGCTTCAATATCCAAAGGAGATCGCAGTAATCAGTGCCCCTCCGGGTTTACCTTAGACTCA
GTTGGACCTTTTTGTGCTGATGAGGATGAATGTGCAGCAGGGAATCCCTGCTCCCATAGCTGCCACAA
TGCCATGGGGACTTACTACTGCTCCTGCCCTAAAGGCCTCACCATAGCTGCAGATGGAAGAACTTGTC
AAGATATTGATGAGTGTGCTTTGGGTAGGCATACCTGCCACGCTGGTCAGGACTGTGACAATACGATT
GGATCTTATCGCTGTGTGGTCCGTTGTGGAAGTGGCTTTCGAAGAACCTCTGATGGGCTGAGTTGTCA
AGATATTAATGAATGTCAAGAATCCAGCCCCTGTCACCAGCGCTGTTTCAATGCCATAGGAAGTTTCC
ATTGTGGATGTGAACCTGGGTATCAGCTCAAAGGCAGAAAATGCATGGATGTGAACGAGTGTAGACAA
AATGTATGCAGACCAGATCAGCACTGTAAGAACACCCGTGGTGGCTATAAGTGCATTGATCTTTGTCC
AAATGGAATGACCAAGGCAGAAAATGGAACCTGTATTGATATTGATGAATGTAAAGATGGGACCCATC
AGTGCAGATATAACCAGATATGTGAGAATACAAGAGGCAGCTATCGTTGTGTATGCCCAAGAGGTTAT
CGGTCTCAAGGAGTTGGAAGACCCTGCATGGATATTGATGAATGTGAAAATACAGATGCCTGCCTGCA
TGAGTGTAAGAATACCTTTGGAAGTTATCAGTGCATCTGCCCACCTGGCTATCAACTCACACACAATG
GAAAGACATGCCAAGATATCGATGAATGTCTGGAGCAGAATGTGCACTGTGGACCCAATCGCATGTGC
TTCAACATGAGAGGAAGCTACCAGTGCATCGATACACCCTGTCCACCCAACTACCAACGGGATCCTGC
TTCAGGGTTCTGCCTCAAGAACTGTCCACCCAATGATTTGGAATGTGCCTTGAGCCCATATGCCTTGG
AATACAAACTCGTCTCCCTCCCATTTGGAATAGCCACCAATCAAGATTTAATCCGGCTGGTTGCATAC
ACACAGGATGGAGTGATGCATCCCAGGACAACTTTCCTCATGGTAGATGAGGAACAGACTGTTCCTTT
TGCCTTGAGGGATGAAAACCTGAAAGGAGTGGTGTATACAACACGACCACTACGAGAAGCAGAGACCT
ACCGCATGAGGGTCCGAGCCTCATCCTACAGTGCCAATGGGACCATTGAATATCAGACCACATTCATA
GTTTATATAGCTGTGTCCGCCTATCCATACTAAGGAACTCTCCAAAGCCTATTCCACATATTTAAACC
GCATTAATCATGGCAATCAAGCCCCCTTCCAGATTACTGTCTCTTGAACAGTTGCAATCTTGGCAGCT
TGAAAATGGTGCTACACTCTGTTTTGTGTGCCTTCCTTGGTACTTCTGAGGTATTTTCATGATCCCAC
CATGGTCATATCTTGAAGTATGGTCTAGAAAAGTCCCTTATTATTTTATTTATTACACTGGAGCAGTT
ACTTCCCAAAGATTATTCTGAACATCTAACAGGACATATCAGTGATGGTTTACAGTAGTGTAGTACCT
AAGATCATTTTCCTGAAAGCCAAACCAAACAACGAAAAACAAGAACAACTAATTCAGAATCAAATAGA
GTTTTTGAGCATTTGACTATTTTTAGAATCATAAAATTAGTTACTAAGTATTTTGATCAAAGCTTATA
AAATAACTTACGGAGATTTTTGTAAGTATTGATACATTATAATAGGACTTGCCTATTTTCATTTTTAA
GAAGAAAAACACCACTCAT

| | | | |
|---|---|---|---|
| NOV43d, CG56914-03 | SEQ ID NO:1004 | 1902 aa | MW at 207163.2 kD |
| Protein Sequence | | | |

MTWMKDGRPLPQTDQVQTLGGGEVLRISTAQVEDTGRYTCLASSPAGDDDKEYLVRVHVPPNIAGTDE
PRDITVLRNRQVTLECKSDAVPPPVITWLRNGERLQATPRVRILSGGRYLQINNADLGDTANYTCVAS
NIAGKTTREFILTVNVPPNIKGGPQSLVILLNKSTVLECIAEGVPTPRITWRKDGAVLAGNHARYSIL
ENGFLHIQSAHVTDTGRYLCMATNAAGTDRRRIDLQVHGSLVIISPSVDDTATYECTVTNGAGDDKRT
VDLTVQVPPSIADEPTDFLVTKHAPAVITCTASGVPFPSIHWTKNGIRLLPRGDGYRILSSGAIElLA
TQLNHAGRYTCVARNAAGSAHRHVTLHVHEPPVIQPQPSELHVILNNPILLPCEATGTPSPFITWQKE
GINVNTSGRNHAVLPSGGLQISRAVREDAGTYMCVAQNPAGTALGKIKLNVQVPPVISPHLKEYVIAV
DKPITLSCEADGLPPPDITWHKDGRAIVESIRQRVLSSGSLQIAFVQPGDAGHYTCMAANVAGSSSTS
TKLTVHVPPRIRSTEGHYTVNENSQAILPCVADGIPTPAINWKKDNVLLANLLGKYTAEPYGELILEN
VVLEDSGFYTCVANNAAGEDTHTVSLTVHVLPTFTELPGDVSLNKGEQLRLSCKATGIPLPKLTWTFN
NNIIPAHFDSVNGHSELVIERVSKEDSGTYVCTAENSVGFVKAIGFVYVKEPPVFKGDYPSNWIEPLG
GNAILNCEVKGDPTPTIQWNRKGVDIEISHRIRQLGNGSLAIYGTVNEDAGDYTCVATNEAGVVERSM
SLTLQSPPIITLEPVETVINAGGKIILNCQATGEPQPTITWSRQGHSISWDDRVNVLSNNSLYIADAQ
KEDTSEFECVARNLMGSVLVRVPVIVQVHGGFSQWSAWRACSVTCGKGIQKRSRLCNQPLPANGGKPC
QGSDLEMRNCQNKPCPVDGSWSEWSLWEECTRSCGRGNQTRTRTCNNPSVQHGGRPCEGNAVEIIMCN
IRPCPVHGAWSAWQPWGTCSESCGKGTQTRARLCNNPPPAFGGSYCDGAETQMQVCNERNCPIHGKWA
TWASWSACSVSCGGGARQRTRGCSDPVPQYGGRKCEGSDVQSDFCNSDPCPTHGNWSPWSGWGTCSRT
CNGGQMRRYRTCDNPPPSNGGRACGGPDSQIQRCNTDMCPVDGSWGSWHSWSQCSASCGGGEKTRKRL
CDHPVPVKGGRPCPGDTTQVTRCNVQACPGGPQRARGSVIGNINDVEFGIAFLNATITDSPNSDTRII
RAKITNVPRSLGSAMRKIVSILNPIYWTTAKEIGEAVNGFTLTNAVFKRETQVEFATGEILQMSHIAR
GLDSDGSLLLDIVVSGYVLQLQSPAEVTVKDYTEDYIQTGPGQLYAYSTRLFTIDGISIPYTWNHTVF
YDQAQGRMPFLVETLHASSVESDYNQIEETLGFKIHASISKGDRSNQCPSGFTLDSVGPFCADEDECA
AGNPCSHSCHNAMGTYYCSCPKGLTIAADGRTCQDIDECALGRHTCHAGQDCDNTIGSYRCVVRCGSG
FRRTSDGLSCQDINECQESSPCHQRCFNAIGSFHCGCEPGYQLKGRKCMDVNECRQNVCRPDQHCKNT
RGGYKCIDLCPNGMTKAENGTCIDIDECKDGTBQCRYNQICENTRGSYRCVCPRGYRSQGVGRPCMDI
DECENTDACLHECKNTFGSYQCICPPGYQLTHNGKTCQDIDECLEQNVHCGPNRNCFNMRGSYQCIDT
PCPPNYQRDPASGFCLKNCPPNDLECALSPYALEYKLVSLPFGIATNQDLIRLVAYTQDGVMHPRTTF
LMVDEEQTVPFALRDENLKGVVYTTRPLREAETYRMRVRASSYSANGTIEYQTTFIVYIAVSAYPY

A ClustalW comparison of the above protein sequences yields the following sequence alignment shown in Table 43B.

TABLE 43B
Comparison of the NOV43 protein sequences.

| | | |
|---|---|---|
| NOV43a | ------------------------------------------------------------ | (SEQ ID NO:998) |
| NOV43b | ------------------------------------------------------------ | (SEQ ID NO:1000) |
| NOV43c | MWLDRCARNPPRNHIDAPQKAPSAFRRVLQKGELISTSSAKFSAGADGSLYVVSPGGEES | (SEQ ID NO:1002) |
| NOV43d | ------------------------------------------------------------ | (SEQ ID NO:1004) |
| NOV43a | ------------------------------------------------------------ | |
| NOV43b | ------------------------------------------------------------ | |
| NOV43c | GEYVCTATNTAGYAKRKVQLTVYVRPRVFGDQRGLSQDKPVEISVLAGEEVTLPCEVKSL | |
| NOV43d | ------------------------------------------------------------ | |
| NOV43a | ------------------------------------------------------------ | |
| NOV43b | ------------------------------------------------------------ | |
| NOV43c | PPPIITWAKETQLISPFSPRHTFLPSGSMKITETRTSDSGMYLCVATNIAGNVTQAVKLN | |
| NOV43d | ------------------------------------------------------------ | |
| NOV43a | ------------------------------------------------------------ | |
| NOV43b | ------------------------------------------------------------ | |
| NOV43c | VHVPPKIQRGPKHLKVQVGQRVDIPCNAQGTPLPVITWSKGGSTMLVDGEHHVSNPDGTL | |
| NOV43d | ------------------------------------------------------------ | |
| NOV43a | ------------------------------------------------------------ | |
| NOV43b | ------------------------------------------------------------ | |
| NOV43c | SIDQATPSDAGIYTCVATNIAGTDETEITLHVQEPPTVEDLEPPYNTTFQERVANQRIEF | |
| NOV43d | ------------------------------------------------------------ | |
| NOV43a | ------------------------------------------------------------ | |
| NOV43b | ------------------------------------------------------------ | |
| NOV43c | PCPAKGTPKPTIKWLHNGRELTGREPGISILEDGTLLVIASVTPYDNGEYICVAVNEAGT | |
| NOV43d | ------------------------------------------------------------ | |
| NOV43a | ------------------------------------------------------------ | |
| NOV43b | ------------------------------------------------------------ | |
| NOV43c | TERKYNLKVHVPPVIKDKEQVTNVSVLLNQLTNLFCEVEGTPSPIIMWYKDNVQVTESST | |
| NOV43d | ------------------------------------------------------------ | |
| NOV43a | ------------------------------------------------------------ | |
| NOV43c | IQTVNNGKILKLFEATPEDAGRYSCKAINIAGTSQKYFNIDVLGTNFPNEVSVVLNRDVA | |
| NOV43d | ------------------------------------------------------------ | |
| NOV43a | ------------------------------------------------------------ | |
| NOV43c | LECQVKGTPFPDIHWFKDGNIKGGNVTTDISVLINSLIKLECETRGLPMPAITWYKDGQP | |
| NOV43d | ------------------------------------------------------------ | |
| NOV43a | ------------------------------------------------------------ | |
| NOV43b | ------------------------------------------------------------ | |
| NOV43c | IMSSSQALYIDKGQYLHIPRAQVSDSATYTCHVANVAGTAEKSFHVDVYVPPMIEGNLAT | |
| NOV43d | ------------------------------------------------------------ | |
| NOV43a | ------------------------------------------------------------ | |
| NOV43b | ------------------------------------------------------------ | |
| NOV43c | PLNKQVVIAHSLTLECKAAGNPSPILTWLKDGVPVKANDNTRIEAGGKKLEIMSAQEIDR | |
| NOV43d | ------------------------------------------------------------ | |
| NOV43a | ------------------------------------------------------------ | |
| NOV43b | ------------------------------------------------------------ | |
| NOV43c | GQYICVATSVAGEKEIKYEVDVLVPPAIEGGDETSYFIVMVNNLLELDCHVTGSPPPTIM | |
| NOV43d | ------------------------------------------------------------ | |
| NOV43a | ------------------------------------------------------------ | |
| NOV43b | ------------------------------------------------------------ | |
| NOV43c | WLKDGQLIDERDGFKILLNGRKLVIAQAQVSNTGLYRCMAANTAGDHKKEFEVTVHVPPT | |
| NOV43d | ------------------------------------------------------------ | |
| NOV43a | ------------------------------------------------------------ | |
| NOV43b | ------------------------------------------------------------ | |
| NOV43c | IKSSGLSERVVVKYKPVALQCIANGIPNPSITWLKDDQPVNTAQGNLKIQSSGRVLQIAK | |
| NOV43d | ------------------------------------------------------------ | |
| NOV43a | ------------------------------------------------------------ | |
| NOV43b | ------------------------------------------------------------ | |
| NOV43c | TLLEDAGRYTCVATNAAGETQQHIQLHVHEPPSLEDAGKMLNETVLVSNPVQLECKAAGN | |
| NOV43d | ------------------------------------------------------------ | |
| NOV43a | ------------------------------------------------------------ | |
| NOV43b | ------------------------------------------------------------ | |
| NOV43c | PVPVITWYKDNRNLSGSTSMTFLNRGQIIDIESAQISDAGIYKCVAINSAGATELFYSLQ | |
| NOV43d | ------------------------------------------------------------ | |
| NOV43a | ------------------------------------------------------------ | |
| NOV43b | ------------------------------------------------------------ | |
| NOV43c | VHVAPSISGSNNMVAVVVNNPVRLECEARGIPAPSLTWLKDGSPVSSFSNGLQVLSGGRI | |
| NOV43d | ------------------------------------------------------------ | |
| NOV43a | ------------------------------------------------------------ | |
| NOV43b | ------------------------------------------------------------ | |
| NOV43c | LALTSAQISDTGRYTCVAVNAAGEKQRDIDLRVYVPPNIMGEEQNVSVLISQAVELLCQS | |
| NOV43d | ------------------------------------------------------------ | |
| NOV43a | ------------------------------------------------------------ | |
| NOV43b | ------------------------------------------------------------ | |
| NOV43c | DAIPPPTLTWLKDGHPLLKKPGLSISENRSVLKIEDAQVQDTGRYTCEATNVAGKTEKNY | |
| NOV43d | ------------------------------------------------------------ | |
| NOV43a | ------------------------------------------------------------ | |
| NOV43b | ------------------------------------------------------------ | |
| NOV43c | NVNIWVPPNIGGSDELTQLTVIEGNLISLLCESSGIPPPNLIWKKKGSPVLTDSMGRVRI | |
| NOV43d | ------------------------------------------------------------ | |
| NOV43a | ------------------------------------------------------------ | |
| NOV43b | ------------------------------------------------------------ | |
| NOV43c | LSGGRQLQISIAEKSDAALYSCVASNVAGTAKKEYNLQVYIRPTITNSGSHPTEIIVTRG | |
| NOV43d | ------------------------------------------------------------ | |
| NOV43a | ------------------------------------------------------------ | |
| NOV43b | ------------------------------------------------------------ | |
| NOV43c | KSISLECEVQGIPPPTVTWMKDGHPLIKAKGVEILDEGHILQLKNIHVSDTGRYVCVAVN | |
| NOV43d | ------------------------------------------------------------ | |
| NOV43a | ------------------------------------------------------------ | |
| NOV43b | ------------------------------------------------------------ | |
| NOV43c | VAGMTDKKYDLSVHGGRMLRLMQTTMEDAGQYTCVVPNAAGEERKIFGLSVLVPPHIVGE | |
| NOV43d | ------------------------------------------------------------ | |
| NOV43a | ------------------------------------------------------------ | |
| NOV43b | ------------------------------------------------------------ | |
| NOV43c | NTLEDVKVKEKQSVTLTCEVTGNPVPEITWHKDGQPLQEDEAHHIISGGRFLQITNVQVP | |
| NOV43d | ------------------------------------------------------------ | |
| NOV43a | ------------------------------------------------------------ | |
| NOV43b | ------------------------------------------------------------ | |
| NOV43c | HTGRYTCLASSPAGHKSRSFSLNVFVSPTIAGVGSDGNPEDVTVILNSPTSLVCEAYSYP | |
| NOV43d | ------------------------------------------------------------ | |
| NOV43a | ------------------------------------------------------------ | |
| NOV43b | ------------------------------------------------------------ | |
| NOV43c | PATITWFKDGTPLESNRNIRILPGGRTLQILNAQEDNAGRYSCVATNEAGEMIKHYEVKV | |
| NOV43d | ------------------------------------------------------------ | |
| NOV43a | ------------------------------------------------------------ | |
| NOV43b | ------------------------------------------------------------ | |
| NOV43c | YTLNANIVIIESQPLKSDDHVNIAANGHTLQIKEAQISDTGRYTCVASNIAGEDELDFDV | |
| NOV43d | ------------------------------------------------------------ | |
| NOV43a | ------------------------------------------------------------ | |
| NOV43b | ------------------------------------------------------------ | |
| NOV43c | NIQVPPSFQKLWEIGNNLDTGRNGEAKDVIINNPISLYCETNAAPPPTLTWYKDGHPLTS | |
| NOV43d | ------------------------------------------------------------ | |
| NOV43a | ------------------------------------------------------------ | |
| NOV43b | ------------------------------------------------------------ | |
| NOV43c | SDKVLILPGGRVLQIPRAKVEDAGRYTCVAVNEAGEDSLQYDVRVLVPPIIKGANSDLPE | |
| NOV43d | ------------------------------------------------------------ | |
| NOV43a | ------------------------------------------------------------ | |
| NOV43b | ------------------------------------------------------------ | |
| NOV43c | EVTVLVNKSALIECLSSGSPAPRNSWQKDGQPLLEDDHHKFLSNGRILQILNTQITDIGR | |
| NOV43d | ------------------------------------------------------------ | |
| NOV43a | ------------------------------------------------------------ | |
| NOV43b | ------------------------------------------------------------ | |
| NOV43c | YVCVAENTAGSAKKYFNLNVHVPPSVIGPKSENLTVVVNNFISLTCEVSGFPPPDLSWLK | |
| NOV43d | ------------------------------------------------------------ | |
| NOV43a | ------------------------------------------------------------ | |
| NOV43b | ------------------------------------------------------------ | |
| NOV43c | NEQPIKLNTNTLIVPGGRTLQIIRAKVSDGGEYTCIAINQAGESKKKFSLTVYVPPSIKD | |
| NOV43d | ------------------------------------------------------------ | |
| NOV43a | ------------------------------------------------------------ | |
| NOV43b | ------------------------------------------------------------ | |
| NOV43c | HDSESLSVVNVREGTSVSLECESNAVPPPVITWYKNGRMITESTHVEILADGQMLHIKKA | |
| NOV43d | ------------------------------------------------------------ | |
| NOV43a | ------------------------------------------------------------ | |
| NOV43b | ------------------------------------------------------------ | |
| NOV43c | EVSDTGQYVCRAINVAGRDDKNFHLNVYVPPSIEGPEREVIVETISNPVTLTCDATGIPP | |
| NOV43d | ------------------------------------------------------------ | |
| NOV43a | ------------------------------------------------------------ | |
| NOV43b | ------------------------------------------------------------ | |
| NOV43c | PTIAWLKNHKRIENSDSLEVRILSGGSKLQIARSQHSDSGNYTCIASNMEGKAQKYYFLS | |
| NOV43d | ------------------------------------------------------------ | |
| NOV43a | ------------------------------------------------------------ | |
| NOV43b | ------------------------------------------------------------ | |
| NOV43c | IQVPPSVAGAEIPSDVSVLLGENVELVCNANGIPTPLIQWLKDGKPIASGETERIRVSAN | |
| NOV43d | ------------------------------------------------------------ | |
| NOV43a | ------------------------------------------------------------ | |
| NOV43b | ------------------------------------------------------------ | |
| NOV43c | GSTLNIYGALTSDTGKYTCVATNPAGEEDRIFNLNVYVTPTIRGNKDEAEKLMTLVDTST | |
| NOV43d | ------------------------------------------------------------ | |
| NOV43a | ------------------------------------------------------------ | |
| NOV43b | ------------------------------------------------------------ | |
| NOV43c | NIECRATGTPPPQINWLKNGLPLPLSSHIRLLAAGQVIRIVRAQVSDVAVYTCVASNRAG | |
| NOV43d | ------------------------------------------------------------ | |
| NOV43a | ------------------------------------------------------------ | |
| NOV43b | ------------------------------------------------------------ | |
| NOV43c | VDNKHYNLQVFAPPNMDNSMGTEEITVLKGSSTSMACITDGTPAPSMAWLRNGQPLGLDA | |
| NOV43d | ------------------------------------------------------------ | |
| NOV43a | ------------------------------------------------------------ | |
| NOV43b | ------------------------------------------------------------ | |
| NOV43c | HLTVSTHGMVLQLLKAETEDSGKYTCIASNEAGEVSKHFILKVLEPPHINGSEEHEEISV | |
| NOV43d | ------------------------------------------------------------ | |
| NOV43a | ------------------------------------------------------------ | |
| NOV43b | ------------------------------------------------------------ | |
| NOV43c | IVNNPLELTCIASGIPAPKMTWMKDGRPLPQTDQVQTLGGGEVLRISTAQVEDTGRYTCL | |
| NOV43d | -------------------MTWMKDGRPLPQTDQVQTLGGGEVLRISTAQVEDTGRYTCL | |
| NOV43a | ------------------------------------------------------------ | |
| NOV43b | ------------------------------------------------------------ | |
| NOV43c | ASSPAGDDDKEYLVRVHVPPNTAGTDEPRDITVLRNRQVTLECKSDAVPPPVITWLRNGE | |
| NOV43d | ASSPAGDDDKEYLVRVHVPPNIAGTDEPRDITVLRNRQVTLECKSDAVPPPVITWLRNGE | |
| NOV43a | ------------------------------------------------------------ | |
| NOV43b | ------------------------------------------------------------ | |
| NOV43c | RLQATPRVRILSGGRYLQINNADLGDTANYTCVASNIAGKTTREFILTVNVPPNIKGGPQ | |
| NOV43d | RLQATPRVRILSGGRYLQINNADLGDTANYTCVASNIAGKTTREFILTVNVPPNIKGGPQ | |
| NOV43a | ------------------------------------------------------------ | |
| NOV43b | ------------------------------------------------------------ | |
| NOV43c | SLVILLNKSTVLECIAEGVPTPRITWRKDGAVLAGNHARYSTLENGFLHIQSAHVTDTGR | |
| NOV43d | SLVILLNKSTVLECIAEGVPTPRITWRKDGAVLAGNHARYSILENGFLHIQSAHVTDTGR | |
| NOV43a | ------------------------------------------------------------ | |
| NOV43b | ------------------------------------------------------------ | |
| NOV43c | YLCMATNAAGTDRIRIDLQVHVPPSIAPGPTNMTVIVNVQTTLACEATGIPKPSINWRKN | |
| NOV43d | YLCMATNAAGTDRRRIDLQVHG-------------------------------------- | |
| NOV43a | ------------------------------------------------------------ | |
| NOV43b | ------------------------------------------------------------ | |
| NOV43c | GHLLNVDQNQNSYRLLSSGSLVIISPSVDDTATYECTVTNGAGDDKRTVDLTVQVPPSIA | |
| NOV43d | -------------------SLVIISPSVDDTATYECTVTNGAGDDKRTVDLTVQVPPSIA | |
| NOV43a | ------------------------------------------------------------ | |
| NOV43b | ------------------------------------------------------------ | |
| NOV43c | DEPTDFLVTKHAPAVITCTASGVPFPSIHWTKNGIRLLPRGDGYRILSSGAIEILATQLN | |
| NOV43d | DEPTDFLVTKHAPAVITCTASGVPFPSIHWTKNGIRLLPRGDGYRILSSGAIEILATQLN | |
| NOV43a | ------------------------------------------------------------ | |
| NOV43b | ------------------------------------------------------------ | |
| NOV43c | HAGRYTCVARNAAGSAHRHVTLHVHEPPVIQPQPSELHVILNNPILLPCEATGTPSPFIT | |
| NOV43d | HAGRYTCVARNAAGSAHRHVTLHVHEPPVIQPQPSELHVILNNPILLPCEATGTPSPFIT | |
| NOV43a | ------------------------------------MCVAQNPAGTALGKIKLNVQVPPV | |
| NOV43b | ---------------------------------GSTMCVAQNPAGTALGKIKLNVQVPPV | |
| NOV43c | WQKEGINVNTSGRNHAVLPSGGLQISRAVREDAGTYMCVAQNPAGTALGKIKLNVQVPPV | |
| NOV43d | WQKEGINVNTSGRNHAVLPSGGLQISRAVREDAGTYMCVAQNPAGTALGKIKLNVQVPPV | |
| NOV43a | ISPHLKEYVIAVDKPITLSCEADGLPPPDITWHKDGRAIVESIRQRVLSSGSLQIAFVQP | |
| NOV43b | ISPHLKEYVIAVDKPITLSCEADGLPPPDITWHKDGRAIVESIRQRVLSSGSLQIAFVQP | |
| NOV43c | ISPHLKEYVIAVDKPITLSCEADGLPPPDITWHKDGRAIVESIRQRVLSSGSLQITFVQP | |
| NOV43d | ISPHLKEYVTAVDKPITLSCEADGLPPPDITWHKDGRAIVESIRQRVLSSGSLQIAFVQP | |
| NOV43a | GDAGHYTCMAANVAGSSSTSTKLTVHVPPRIRSTEGHYTVNENSQAILPCVADGIPTPAI | |
| NOV43b | GDAGHYTCMAANVAGSSSTSTKLTVHVPPRIRSTEGHYTVNENSQAILPCVADGIPTPAI | |
| NOV43c | GDAGHYTCMAANVAGSSSTSTKLTVHVPPRIRSTEGHYTVNENSQAILPCVADGIPTPAI | |
| NOV43d | GDAGHYTCMAANVAGSSSTSTKLTVHVPPRIRSTEGHYTVNENSQAILPCVADGIPTPAI | |
| NOV43a | NWKKDNVLLANLLGKYTAEPYGELILENVVLEDSGFYTCVANNAAGEDTHTVSLTVHVLP | |
| NOV43b | NWKKDNVLLANLLGKYTAEPYGELILENVVLEDSGFYTCVANNAAGEDTHTVSLTVHVLP | |
| NOV43c | NWKKDNVLLANLLGKYTAEPYGELILENVVLEDSGFYTCVANNAAGEDTHTVSLTVHVLP | |
| NOV43d | NWKKDNVLLANLLGKYTAEPYGELILENVVLEDSGFYTCVANNAAGEDTHTVSLTVHVLP | |
| NOV43a | TFTELPGDVSLNKGEQLRLSCKATGIPLPKLTWTFNNNIIPAHFDSVNGHSELVIERVSK | |
| NOV43b | TFTELPGDVSLNKGEQLRLSCKATGIPLPKLTWTFNNNIIPAHFDSVNGHSELVIERVSK | |
| NOV43c | TFTELPGDVSLNKGEQLRLSCKATGIPLPKLTWTFNNNIIPAHFDSVNGHSELVIERVSK | |
| NOV43d | TFTELPGDVSLNKGEQLRLSCKATGIPLPKLTWTFNNNIIPAHFDSVNGHSELVIERVSK | |
| NOV43a | EDSGTYVCTAENSVGFVKAIGFVYVKEPPVFKGDYPSNWIEPLGGNAILNCEVKGDPTPT | |
| NOV43b | EDSGTYVCTAENSVGFVKAIGFVYVKEPPVFKGDYPSNWIEPLGGNAILNCEVKGDPTPT | |
| NOV43c | EDSGTYVCTAENSVGFVKAIGFVYVKEPPVFKGDYPSHWIEPLGGNAILNCEVKGDPTPT | |
| NOV43d | EDSGTYVCTAENSVGFVKAIGFVYVKEPPVFKGDYPSNWIEPLGGNAILNCEVKGDPTPT | |
| NOV43a | IQWNRKGVDIEISHRIRQLGNGSLAIYGTVNEDAGDYTCVATNEAGVVERSMSLTLQSPP | |
| NOV43b | IQWNRKGVDIEISHRIRQLGNGSLAIYGTVNEDAGDYTCVATNEAGVVERSMSLTLRSPP | |
| NOV43c | IQWNRKGVDIEISHRIRQLGNGSLAIYGTVNEDAGDYTCVATNEAGVVERSMSLTLQSPP | |
| NOV43d | IQWNRKGVDIEISHRIRQLGNGSLAIYGTVNEDAGDYTCVATNEAGVVERSMSLTLQSPP | |
| NOV43a | IITLEPVETVINAGGKIILNCQATGEPQPTITWSRQGHSISWDDRVNVLSNNSLYIADAQ | |
| NOV43b | IITLEPVETVINAGGKIILNCQATGEPQPTITWSRQGHSISWDDRVNVLSNNSLYIADAQ | |
| NOV43c | IITLEPVETVINAGGKIILNCQATGEPQPTITWSRQGHSISWDDRVNVLSNNSLYIADAQ | |
| NOV43d | IITLEPVETVINAGGKIILNCQATGEPQPTITWSRQGHSISWDDRVNVLSNNSLYIADAQ | |
| NOV43a | KEDTSEFECVARNLMGSVLVRVPVIVQVHGGFSQWSAWRACSVTCGKGIQKRSRLCNQPL | |
| NOV43b | KEDTSEFECVARNLMGSVLVRVPVIVQVHGGFSQWSAWRACSVTCGKGIQKRSRLCNQPL | |
| NOV43c | KEDTSEFECVARNLMGSVLVRVPVIVQVHGGFSQWSAWRACSVTCGKGIQKRSRLCNQPL | |
| NOV43d | KEDTSEFECVARNLMGSVLVRVPVIVQVHGGFSQWSAWPACSVTCGKGIQKRSRLCNQPL | |
| NOV43a | PANGGKPCQGSDLEMRNCQNKPCPVDGSWSEWSLWEECTRSCGRGNQTRTRTCNNPSVQH | |
| NOV43b | PANGGKPCQGSDLEMRNCQNKPCPVDGSWSEWSLWEECTRSCGRGNQTRTRTCNNPSVQH | |
| NOV43c | PANGGKPCQGSDLEMRNCQNKPCPVDGSWSEWSLWEECTRSCGRGNQTRTRTCNNPSVQH | |
| NOV43d | PANGGKPCQGSDLEMPNCQNKPCPVDGSWSEWSLWEECTRSCGRGNQTRTRTCNNPSVQH | |
| NOV43a | GGRPCEGNAVEIIMCNIRPCPVHGAWSAWQPWGTCSESCGKGTQTRARLCNNPPPAFGGS | |
| NOV43b | GGRPCEGNAVEIIMCNIRPCPVHGAWSAWQLWGTCSESCGKGTQTRARLCNNPPPAFGGS | |
| NOV43c | GGRPCEGNAVEIIMCNIRPCPVHGAWSAWQPWGTCSESCGKGTQTRARLCNNPPPAFGGS | |
| NOV43d | GGRPCEGNAVEIIMCNIRPCPVHGAWSAWQPWGTCSESCGKGTQTRARLCNNPPPAFGGS | |
| NOV43a | YCDGAETQMQVCNERICPIHGKWATWASWSACSVSCGGGARQRTRGCSDPVPQYGGRKCE | |
| NOV43b | YCDGAETQMQVCNERNCPIHGKWATWASWSACSVSCGGGARQRTRGCSDPVPQYGGRKCE | |
| NOV43c | YCDGAETQMQVCNERNCPVHGKWATWASWSACSVSCGGGARQRTRGCSDPVPQYGGRKCE | |
| NOV43d | YCDGAETQMQVCNERNCPIHGKWATWASWSACSVSCGGGARQRTRGCSDPVPQYGGRKCE | |
| NOV43a | GSDVQSDFCNSDPCPSECWKYPW------------------------------------- | |
| NOV43b | GSDVQSDFCNSDPCPSECWKYPWLE----------------------------------- | |
| NOV43c | GSDVQSDFCNSDPCPTHGNWSPWSGWGTCSRTCNGGQMRRYRTCDNPPPSNGGRACGGPD | |
| NOV43d | GSDVQSDFCNSDPCPTHGNWSPWSGWGTCSRTCNGGQMRRYRTCDNPPPSNGGRACGGPD | |
| NOV43a | ------------------------------------------------------------ | |
| NOV43b | ------------------------------------------------------------ | |
| NOV43c | SQIQRCNTDMCPVDGSWGSWHSWSQCSASCGGGEKTRKRLCDHPVPVKGGRPCPGDTTQV | |
| NOV43d | SQIQRCNTDMCPVDGSWGSWHSWSQCSASCGGGEKTRKRLCDHPVPVKGGRPCPGDTTQV | |
| NOV43a | ------------------------------------------------------------ | |
| NOV43b | ------------------------------------------------------------ | |
| NOV43c | TRCNVQACPGGPQRARGSVIGNINDVEFGIAFLNATITDSPNSDTRIIRAKITNVPRSLG | |
| NOV43d | TRCNVQACPGGPQRARGSVIGNINDVEFGIAFLNATITDSPNSDTRIIRAKITNVPRSLG | |
| NOV43a | ------------------------------------------------------------ | |
| NOV43b | ------------------------------------------------------------ | |
| NOV43c | SAMRKIVSILNPIYWTTAKEIGEAVNGFTLTNAVFKRETQVEFATGEILQMSHIARGLDS | |
| NOV43d | SAMRKIVSILNPIYWTTAKEIGEAVNGFTLTNAVFKRETQVEFATGEILQMSHIARGLDS | |
| NOV43a | ------------------------------------------------------------ | |
| NOV43b | ------------------------------------------------------------ | |
| NOV43c | DGSLLLDIVVSGYVLQLQSPAEVTVKDYTEDYIQTGPGQLYAYSTRLFTIDGISIPYTWN | |
| NOV43d | DGSLLLDIVVSGYVLQLQSPAEVTVKDYTEDYIQTGPGQLYAYSTRLFTIDGISIPYTWN | |
| NOV43a | ------------------------------------------------------------ | |
| NOV43b | ------------------------------------------------------------ | |
| NOV43c | HTVFYDQAQGRNPFLVETLHASSVESDYNQIEETLGFKTHASISKGDRSNQCPSGFTLDS | |
| NOV43d | HTVFYDQAQGRMPFLVETLHASSVESDYNQIEETLGFKIHASISKGDRSNQCPSGFTLDS | |
| NOV43a | ------------------------------------------------------------ | |
| NOV43b | ------------------------------------------------------------ | |
| NOV43c | VGPFCADEDECAAGNPCSHSCHNAMGTYYCSCPKGLTIAADGRTCQDIDECALGRRTCHA | |
| NOV43d | VGPFCADEDECAAGNPCSHSCHNAMGTYYCSCPKGLTIAADGRTCQDIDECALGRHTCHA | |
| NOV43a | ------------------------------------------------------------ | |
| NOV43b | ------------------------------------------------------------ | |
| NOV43c | GQDCDNTIGSYRCVVRCGSGFRRTSDGLSCQDINECQESSPCHQRCFNAIGSFHCGCEPG | |
| NOV43d | GQDCDNTIGSYRCVVRCGSGFRRTSDGLSCQDINECQESSPCHQRCFNAIGSFHCGCEPG | |
| NOV43a | ------------------------------------------------------------ | |
| NOV43b | ------------------------------------------------------------ | |
| NOV43c | YQLKGRKCMDVNECRQNVCRPDQHCKNTRGGYKCIDLCPNGMTKAENGTCIDIDECKDGT | |
| NOV43d | YQLKGRKCMDVNECRQNVCRPDQHCKNTRGGYKCIDLCPNGMTKAENGTCIDIDECKDGT | |
| NOV43a | ------------------------------------------------------------ | |
| NOV43b | ------------------------------------------------------------ | |
| NOV43c | HQCRYNQICENTRGSYRCVCPRGYRSQGVGRPCMDINECEQVPKPCAHQCSNTPGSFKCI | |
| NOV43d | HQCRYNQICENTRGSYRCVCPRGYRSQGVGRPCMDI------------------------ | |
| NOV43a | ------------------------------------------------------------ | |
| NOV43b | ------------------------------------------------------------ | |
| NOV43c | CPPGQHLLGDGKSCAGLERLPNYGTQYSSYNLARFSPVRNNYQPQQHYRQYSHLYSSYSE | |
| NOV43d | ------------------------------------------------------------ | |
| NOV43a | ------------------------------------------------------------ | |
| NOV43b | ------------------------------------------------------------ | |
| NOV43c | YRNSRTSLSRTRRTIRKTCPEGSEASHDTCVDIDECENTDACQHECKNTFGSYQCICPPG | |
| NOV43d | ---------------------------------DECENTDACLHECKNTFGSYQCICPPG | |
| NOV43a | ------------------------------------------------------------ | |
| NOV43b | ------------------------------------------------------------ | |
| NOV43c | YQLTHNGKTCQDIDECLEQNVHCGPNRMCFNMRGSYQCIDTPCPPNYQRDPVSGFCLKNC | |
| NOV43d | YQLTHNGKTCQDIDECLEQNVHCGPNRMCFNMRGSYQCIDTPCPPNYQRDPASGFCLKNC | |
| NOV43a | ------------------------------------------------------------ | |
| NOV43b | ------------------------------------------------------------ | |
| NOV43c | PPNDLECALSPYALEYKLVSLPFGIATNQDLIRLVAYTQDGVMHPRTTFLMVDEEQTVPF | |
| NOV43d | PPNDLECALSPYALEYKLVSLPFGIATNQDLIRLVAYTQDGVMHPRTTFLMVDEEQTVPF | |
| NOV43a | ------------------------------------------------------- | |
| NOV43b | ------------------------------------------------------- | |
| NOV43c | ALRDENLKGVVYTTRPLREAETYRMRVRASSYSANGTIEYQTTFIVYIAVSAYPY | |
| NOV43d | ALRDENLKGVVYTTRPLREAETYRNRVRASSYSANGTIEYQTTFIVYIAVSAYPY | |

Further analysis of the NOV43a protein yielded the following properties shown in Table 43C.

TABLE 43C
Protein Sequence Properties NOV43a

| | |
|---|---|
| SignalP analysis: | No Known Signal Sequence Predicted |
| PSORT II analysis: | |

| PSG: | a new signal peptide prediction method | |
|---|---|---|
| | N-region: | length 0; pos. chg 0; neg. chg 0 |
| | H-region: | length 13; peak value 2.20 |
| | PSG score: | −2.20 |

| | |
|---|---|
| GvH: | von Heijne's method for signal seq. recognition |
| | GvH score (threshold: −2.1): −8.62 |
| | possible cleavage site: between 48 and 49 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 0 | |
| | number of TMS(s) . . . fixed | |
| | PERIPHERAL | Likelihood = 0.63 (at 454) |

| | ALOM score: 0.63 (number of TMSs: 0) | | | |
|---|---|---|---|---|
| MITDISC: | discrimination of mitochondrial targeting seq | | | |
| | R content: | 0 | Hyd Moment(75): | 4.71 |
| | Hyd Moment(95): | 3.65 | G content: | 2 |
| | D/E content: | 1 | S/T content: | 2 |

| | |
|---|---|
| | Score: −5.83 |
| Gavel: | prediction of cleavage sites for mitochondrial preseq |
| | cleavage site motif not found |

NUCDISC: discrimination of nuclear localization signals

| | |
|---|---|
| | pat4: none |
| | pat7: none |
| | bipartite: none |
| | content of basic residues: 8.5% |
| | NLS Score: −0.47 |
| KDEL: | ER retention motif in the C-terminus: none |

ER Membrane Retention Signals:

KKXX-like motif in the C-terminus: WKYP

SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear |
| | discrimination |
| | Prediction: nuclear |
| | Reliability: 55.5 |
| COIL: | Lupas's algorithm to detect coiled-coil regions |
| | total: 0 residues |

Final Results (k = 9/23):

73.9%: nuclear
13.0%: mitochondrial
13.0%: cytoplasmic

>> prediction for CG56914-01 is nuc (k = 23)

A search of the NOV43a protein against the Geneseq database, a proprietary database that contains sequences published in patents and patent publication, yielded several homologous proteins shown in Table 43D.

TABLE 43D
Geneseq Results for NOV43a

| | | NOV43a | Identities/ | | |
|---|---|---|---|---|---|
| | | Residues/ | Similarities for | | |
| Geneseq | Protein/Organism/Length | Match | the Matched | | Expect |
| Identifier | [Patent #, Date] | Residues | Region | | Value |
| ABU12084 | Human NOV25c CG56914-03 protein | 1 . . . 707 | 701/707 | (99%) | 0.0 |
| | SEQ ID 88 - Homo sapiens, 1902 aa. | 441 . . . 1147 | 702/707 | (99%) | |
| | [WO200281625-A2, 17-OCT-2002] | | | | |
| ABP60991 | Novel human protein. SEQ ID 78 - | 1 . . . 707 | 701/707 | (99%) | 0.0 |
| | Homo sapiens, 5635 aa. | 4057 . . . 4763 | 702/707 | (99%) | |
| | [WO200250105-A1, 27-JUN-2002] | | | | |
| AAB47771 | Human thrombospondin protein, | 1 . . . 707 | 699/707 | (98%) | 0.0 |
| | BTL.012 - Homo sapiens, 1336 aa. | 184 . . . 890 | 702/707 | (98%) | |
| | [WO200174852-A2, 11-OCT-2001] | | | | |
| ABU12083 | Human NOV25b CG93858-02 protein | 1 . . . 707 | 698/707 | (98%) | 0.0 |
| | SEQ ID 86 - Homo sapiens, 2572 aa. | 1111 . . . 1817 | 701/707 | (98%) | |
| | [WO200281625-A2, 17-OCT-2002] | | | | |
| ABG03933 | Novel human diagnostic protein #3924 - | 1 . . . 471 | 471/471 | (100%) | 0.0 |
| | Homo sapiens, 1240 aa. | 442 . . . 912 | 471/471 | (100%) | |
| | [WO200175067-A2, 11-OCT-2001] | | | | |

In a BLAST search of public sequence databases, the NOV43a protein was found to have homology to the proteins shown in the BLASTP data in Table 43E.

TABLE 43E
Public BLASTP Results for NOV43a

| | | NOV43a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96RW7 | Hemicentin - Homo sapiens | 1 . . . 707 | 700/707 (99%) | 0.0 |
| | (Human), 5636 aa. | 4058 . . . 4764 | 701/707 (99%) | |
| Q96SC3 | Fibulin-6 - Homo sapiens (Human), | 1 . . . 707 | 698/707 (98%) | 0.0 |
| | 2673 aa (fragment). | 1095 . . . 1801 | 701/707 (98%) | |
| Q8NDA2 | Hypothetical protein - Homo sapiens | 2 . . . 489 | 208/503 (41%) | e−111 |
| | (Human), 1340 aa (fragment). | 824 . . . 1326 | 285/503 (56%) | |
| Q96DN3 | Hypothetical protein FLJ31995 - | 1 . . . 460 | 159/475 (33%) | 1e−63 |
| | Homo sapiens (Human), 1252 aa | 782 . . . 1252 | 235/475 (49%) | |
| | (fragment). | | | |
| Q8I0L3 | C. elegans him-4 protein | 2 . . . 511 | 159/529 (30%) | 1e−59 |
| | (corresponding sequence F15G9.4a) - | 3014 . . . 3521 | 241/529 (45%) | |
| | Caenorhabditis elegans, 5175 aa. | | | |

PFam analysis predicts that the NOV43a protein contains the domains shown in the Table 43F.

TABLE 43F
Domain Analysis of NOV43a

| | | Identities/ | |
|---|---|---|---|
| Pfam | NOV43a | Similarities for | Expect |
| Domain | Match Region | the Matched Region | Value |
| ig | 37 . . . 94 | 21/61 (34%) | 1.1e−09 |
| | | 44/61 (72%) | |
| ig | 127 . . . 185 | 17/62 (27%) | 3.2e−06 |
| | | 38/62 (61%) | |
| ig | 218 . . . 274 | 20/60 (33%) | 1.3e−10 |
| | | 44/60 (73%) | |
| ig | 308 . . . 365 | 21/61 (34%) | 5.3e−09 |
| | | 43/61 (70%) | |
| ig | 398 . . . 455 | 18/61 (30%) | 3.5e−08 |
| | | 42/61 (69%) | |
| tsp_1 | 477 . . . 527 | 28/53 (53%) | 8.9e−17 |
| | | 37/53 (70%) | |
| tsp_1 | 534 . . . 584 | 25/53 (47%) | 4.8e−14 |
| | | 42/53 (79%) | |
| tsp_1 | 591 . . . 641 | 22/53 (42%) | 2.7e−12 |
| | | 36/53 (68%) | |
| tsp_1 | 648 . . . 698 | 23/53 (43%) | 1.7e−14 |
| | | 37/53 (70%) | |

### Example 44

The NOV44 clone was analyzed, and the nucleotide and encoded polypeptide sequences are shown in Table 44A.

TABLE 44A
NOV44 Sequence Analysis

| | | |
|---|---|---|
| NOV44a, CG56959-02 | SEQ ID NO: 1005 | 1599 bp |
| DNA Sequence | ORF Start: ATG at 25 | ORF Stop: TAA at 1594 |

GACTCACGAAAGAAAAGCCAAACGATGAGTTTCCACAAGGAGGACGGAGTGAACAGTCTGTGCCAGAA
GGCTCTGCACATCGTCACCGAGCTGTGCTTCGCCGGCCAGGTGGAGTGGGAGAAGTGCTCGGGCATCT
TCCCTCGGGACAGGGGCAGCCAGGGCGGAAGCAGCACAGATATTTCAGTCAGCCTGTTAGCAGTCGTT
GTCAGCTTTTGTGGACTGGCCTTGTTGGTTGTCTCACTTTTTGTCTTCTGGAAGCTGTGTTGGCCATG
CTGGAAAAGCAAACCTGTGACTTCCAACATCACTACCCTTCCACAGAGCATTTCAAGTGCTCCTACTG
AAGTTTTTGAGACTGAAGAGAAAAAAGAAATTAAGGAAAATGAAAAGCCAGCCGTAAAAGCTATTGAG
CCTGCAATAAAAATCAGCCACACTTCCCCTGACATCCCAGCAGAAGTCCAAACTGCTTTAAAAGAACA
TTTAATTAAACATGCACGTGTGCAAAGACAAATTACTGAGCCTACGTCATCAACCCGCCACAGTTCCT
TCCGAAGACACCTGCCGAGGCAAATGCAGGTTTCCAGTGTTGATTTTAGCATGGGCACAGAACCTGTT
TTACAACGAGGAGAAACAACAACCAGCATTGGGAGGATAAAGCCAGAACTCTACAAACAGAAATCAGT
TGACTCTGAGGGCAACCAAAACGAAGATGTCAAAATCTGTGGGAAACTTAACTTTACCCTCCAGTATG
ATTATGAAAATGAACTTCTAGTTGTTAAAATTATCAAAGCTTTAGATCTCCCTGCTAAAGACTTCACA
GGAACTTCTGACCCTTATGTGAAGATGTATCTTCTTCCAGATAGGAAAAAGAAATTTCAGACCCGCGT
GCACAGAAAGACTTTAAATCCTCTATTTGATGAAACTTTTCAATTTCCTGTAGCATATGATCAACTAA
GCAACCGAAAACTACATTTCAGTGTGTATGATTTTGACAGATTTTCTAGACATGACATGATTGGGGAA
GTGATTCTTGATAATTTGTTTGAAGTCTCTGATCTCTCCAGGGAAGCCACAGTATGGAAAGATATTCA
CTGTGCTACCACAGAAAGTATAGACCTGGGTGAAATCATGTTTTCCCTTTGTTACCTACCGACGGCTG
GGCGTATGACATTGACAGTCATTAAGTGCAGAAATCTGAAGGCGATGGATATTACTGGCTCATCAGAT
CCTTATGTCAAAGTGTTCCTGATGTGTGAAGGTCGAAGATTAAAAAAGAGGAAAACAACTACAAAGAA
AAACACTCTAAACCCTGTGTACAATGAGGCCATTATTTTTGACATCCCTCCAGAGAACGTGGACCAGG
TCAGCCTCTCCATTGCGGTCATGGATTACGATAGGGTAGGACACAATGAGGTCATAGGAGTGTGCAGA
ACAGGACTGGATGCTGAGGGTCTTGGGCGAGACCACTGGAATGAAATGCTGGCCTATCATCGAAAACC
AATAACGCACTGGCACCCATTGCTGGAGTTACCTGGCCGGGCGACCAGTTTTGATAGTCAAGGATCCT
GCCCTTCTCCTAAACCACCTTCCACACCATAATGC

| | | | |
|---|---|---|---|
| NOV44a, CG56959-02 | SEQ ID NO: 1006 | 523 aa | MW at 59186.2kD |
| Protein Sequence | | | |

MSFHKEDGVNSLCQKALHIVTELCFAGQVEWEKCSGIFPRDRGSQGGSSTDISVSLLAVVVSFCGLAL
LVVSLFVFWKLCWPCWKSKPVTSNITTLPQSISSAPTEVFETEEKKEIKENEKPAVKAIEPAIKISHT
SPDIPAEVQTALKEHLIKHARVQRQITEPTSSTRHSSFRRHLPRQMQVSSVDFSMGTEPVLQRGETTT
SIGRIKPELYKQKSVDSEGNQNEDVKICGKLNFTLQYDYENELLVVKIIKALDLPAKDFTGTSDPYVK
MYLLPDRKKKFQTRVHRKTLNPLFDETFQFPVAYDQLSNRKLHFSVYDFDRFSRHDMIGEVILDNLFE
VSDLSREATVWKDIHCATTESIDLGEIMFSLCYLPTAGPMTLTVIKCRNLKAMDITGSSDPYVKVFLM
CEGRRLKKRKTTTKKNTLNPVYNEAIIFDIPPENVDQVSLSIAVMDYDRVGHNEVIGVCRTGLDAEGL
GRDHWNEMLAYHRKPITHWHPLLELPGRATSFDSQGSCPSPKPPSTP

| | | |
|---|---|---|
| NOV44b, CG56959-01 | SEQ ID NO: 1007 | 1697 bp |
| DNA Sequence | ORF Start: ATG at 78 | ORF Stop: TAA at 1647 |

CAGAGGGGTTAGAGGTACGGGAAGAGGAAAAGACGGCTAACTGGGAAAAAAAGAGAAAACGAAAGAAA
AGCCAAACGATGAGTTTCCACAAGGAGGACGGAGTGAACAGTCTGTGCCAGAAGGCTCTGCACATCGT
CACCGAGCTGTGCTTCGCCGGCCAGGTGGAGTGGGAGAAGTGCTCGGGCATCTTCCCTCGGGACAGGG
GCAGCCAGGGCGGAAGCAGCACAGATATTTCAGTCAGCCTGTTAGCTGTCGTTGTCAGCTTTTGTGGA
CTGGCCTTGTTGGTTGTCTCACTTTTTGTCTTCTGGAAGCTGTGTTGGCCATGCTGGAAAAGCAAACC
TGTGACTTCCAACATCACTACGCTTCCACAGAGCATTTCAAGTGCTCCTACTGAAGTTTTTGAGACTG
AAGAGAAAAAAGAAATTAAGGAAAATGAAAAGCCAGCCGTAAAAGCTATTGAGCCTGCAATAAAAATC
AGCCACACTTCCCCTGACATCCCAGCAGAAGTCCAAACTGCTTTAAAAGAACATTTAATTAAACATGC
ACGTGTGCAAAGACAAATTACTGAGCCTACGTCATCAACCCGGCACAGTTCCTTCCGAAGACACCTGC
CGAGGCAAATGCAGGTTTCCAGTGTTGATTTTAGCATGGGCACAGAACCTGTTTTACAACGAGGAGAA
ACAACAACCAGCATTGGGAGGATAAAGCCAGAACTCTACAAACAGAAATCAGTTGACTCTGAGGGCAA
CCAAAACGAAGATGTCAAAATCTGTGGGAAACTTAACTTTACCCTCCAGTATGATTATGAAAATGAAC
TTCTAGTTGTTAAAATTATCAAAGCTTTAGATCTCCCTGCTAAAGACTTCACAGGAACTTCTGACCCT
TATGTGAAGATGTATCTTCTTCCAGATAGGAAAAAGAAATTTCAGACCCGCGTGCACAGAAAGACTTT
AAATCCTCTATTTGATGAAACTTTTCAATTTCCTGTAGCATATGATCAACTAAGCAACCGAAAACTAC
ATTTCAGTGTGTATGATTTTGACAGATTTTCTAGACATGACATGATTGGGGAAGTGATTCTTGATAAT
TTGTTTGAAGTCTCTGATCTCTCCAGGGAAGCCACAGTATGGAAAGATATTCACTGTGCTACCACAGA
AAGTATAGACCTGGGTGAAATCATGTTTTCCCTTTGTTACCTACCGACGGCTGGGCGTATGACATTGA
CAGTCATTAAGTGCAGAAATCTGAAGGCGATGGATATTACTGGCTCATCAGATCCTTATGTCAAAGTG
TCCCTGATGTGTGAAGGTCGAAGATTAAAAAAGAGGAAAACAACTACAAAGAAAAACACTCTAAACCC
TGTGTACAATGAGGCCATTATTTTTGACATCCCTCCAGAGAACGTGGACCAGGTCAGCCTCTCCATTG
CGGTCATGGATTACGATAGGGTAGGACACAATGAGGTCATAGGAGTGTGCAGAACAGGACTGGATGCT
GAGGGTCTTGGGCGAGACCACTGGAATGAAATGCTGGCCTATCATCGAAAACCAATAACGCACTGGCA
CCCATTGCTGGAGTTACCTGGCCGGGCGACCAGTTTTGATAGTCAAGGATCCTGCCCTTCTCCTAAAC
CACCTTCCACACCATAATGCCTCCAAAATGAGACCATGATATTAAGCATCTAGGATCACGTGCTC

| | | | |
|---|---|---|---|
| NOV44b, CG56959-01 | SEQ ID NO: 1008 | 523 aa | MW at 59126.1kD |
| Protein Sequence | | | |

MSFHKEDGVNSLCQKALHIVTELCFAGQVEWEKCSGIFPRDRGSQGGSSTDISVSLLAVVVSFCGLAL
LVVSLFVFWKLCWPCWKSKPVTSNITTLPQSISSAPTEVFETEEKKEIKENEKPAVKAIEPAIKISHT
SPDIPAEVQTALKEHLIKHARVQRQITEPTSSTRHSSFRRHLPRQMQVSSVDFSMGTEPVLQRGETTT
SIGRIKPELYKQKSVDSEGNQNEDVKICGKLNFTLQYDYENELLVVKIIKALDLPAKDFTGTSDPYVK
MYLLPDRKKKFQTRVHRKTLNPLFDETFQFPVAYDQLSNRKLHFSVYDFDRFSRHDMIGEVILDNLFE
VSDLSREATVWKDIHCATTESIDLGEIMFSLCYLPTAGRMTLTVIKCRNLKAMDITGSSDPYVKVSLM
CEGRRLKKRKTTTKKNTLNPVYNEAIIFDIPPENVDQVSLSIAVMDYDRVGHNEVIGVCRTGLDAEGL
GRDHWNEMLAYHRKPITHWHPLLELPGRATSFDSQGSCPSPKPPSTP

A ClustalW comparison of the above protein sequences yields the following sequence alignment shown in Table 44B.

TABLE 44B
Comparison of the NOV44 protein sequences.

| | |
|---|---|
| NOV44a | MSFHKEDGVNSLCQKALHIVTELCFAGQVEWEKCSGIFPRDRGSQGGSSTDISVSLLAVV |
| NOV44b | MSFHKEDGVNSLCQKALHIVTELCFAGQVEWEKCSGIFPRDRGSQGGSSTDISVSLLAVV |
| NOV44a | VSFCGLALLVVSLFVFWKLCWPCWKSKPVTSNITTLPQSISSAPTEVFETEEKKEIKENE |
| NOV44b | VSFCGLALLVVSLFVFWKLCWPCWKSKPVTSNITTLPQSISSAPTEVFETEEKKEIKENE |
| NOV44a | KPAVKAIEPAIKISHTSPDIPAEVQTALKEHLIKHARVQRQITEPTSSTRHSSFRRHLPR |
| NOV44b | KPAVKAIEPAIKISHTSPDIPAEVQTALKEHLIKHARVQRQITEPTSSTRHSSFRRHLPR |
| NOV44a | QMQVSSVDFSMGTEPVLQRGETTTSIGRIKPELYKQKSVDSEGNQNEDVKICGKLNFTLQ |
| NOV44b | QMQVSSVDFSMGTEPVLQRGETTTSIGRIKPELYKQKSVDSEGNQNEDVKICGKLNFTLQ |
| NOV44a | YDYENELLVVKIIKALDLPAKDFTGTSDPYVKMYLLPDRKKKFQTRVHRKTLNPLFDETF |
| NOV44b | YDYENELLVVKIIKALDLPAKDFTGTSDPYVKMYLLPDRKKKFQTRVHRKTLNPLFDETF |
| NOV44a | QFPVAYDQLSNRKLHFSVYDFDRFSRHDMIGEVILDNLFEVSDLSREATVWKDIHCATTE |
| NOV44b | QFPVAYDQLSNRKLHFSVYDFDRFSRHDMIGEVILDNLFEVSDLSREATVWKDIHCATTE |
| NOV44a | SIDLGEIMFSLCYLPTAGRMTLTVIKCFNLKAMDITGSSDPYVKVFLMCEGPRLKKRKTT |
| NOV44b | SIDLGEIMFSLCYLPTAGRMTLTVIKCRNLKAMDITGSSDPYVKVSLMCEGPRLKKRKTT |
| NOV44a | TKKNTLNPVYNEAIIFDIPPENVDQVSLSIAVMDYDRVGHNEVIGVCRTGLDAEGLGRDH |
| NOV44b | TKKNTLNPVYNEAIIFDIPPENVDQVSLSIAVMDYDRVGHNEVIGVCRTGLDAEGLGRDH |
| NOV44a | WNEMLAYHRKPITHWHPLLELPGRATSFDSQGSCPSPKPPSTP |
| NOV44b | WNEMLAYHRKPITHWHPLLELPGRATSFDSQGSCPSPKPPSTP |
| NOV44a | (SEQ ID NO: 1006) |
| NOV44b | (SEQ ID NO: 1008) |

Further analysis of the NOV44a protein yielded the following properties shown in Table 44C.

TABLE 44C
Protein Sequence Properties NOV44a

| | |
|---|---|
| SignalP analysis: | Cleavage site between residues 32 and 33 |
| PSORT II analysis: | |

| PSG: | a new signal peptide prediction method | |
|---|---|---|
| | N-region: | length 7; pos. chg 1; neg. chg 2 |
| | H-region: | length 7; peak value 0.00 |
| | PSG score: | −4.40 |

| | |
|---|---|
| GvH: | von Heijne's method for signal seq. recognition |
| | GvH score (threshold: −2.1): −7.65 |
| | possible cleavage site: between 28 and 29 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 1 | | |
| | Number of TMS(s) for threshold 0.5: 1 | | |
| | INTEGRAL | Likelihood = | Transmembrane 59-75 |
| | | −11.36 | |

| | |
|---|---|
| PERIPHERAL | Likelihood = 4.35 (at 247) |

| | |
|---|---|
| | ALOM score: −11.36 (number of TMSs: 1) |
| MTOP: | Prediction of membrane topology (Hartmann et al.) |
| | Center position for calculation: 66 |
| | Charge difference: 3.0 C(3.0)-N(0.0) |
| | C > N: C-terminal side will be inside |

>>> membrane topology: type 1b (cytoplasmic tail 59 to 523)

| MITDISC: | discrimination of mitochondrial targeting seq | | | |
|---|---|---|---|---|
| | R content: | 0 | Hyd Moment(75): | 5.17 |
| | Hyd Moment(95): | 1.90 | G content: | 0 |
| | D/E content: | 2 | S/T content: | 1 |

| | |
|---|---|
| | Score: −7.37 |
| Gavel: | prediction of cleavage sites for mitochondrial preseq |
| | cleavage site motif not found |

NUCDISC: discrimination of nuclear localization signals

| | |
|---|---|
| | pat4: RKKK (5) at 279 |
| | pat4: KKRK (5) at 415 |
| | pat7: PDRKKKF (5) at 277 |
| | bipartite: none |
| | content of basic residues: 12.6% |
| | NLS Score: 0.58 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: found

LL at 68
LL at 247
LL at 275
LL at 498

checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 55.5 |
| COIL: | Lupas's algorithm to detect coiled-coil regions |
| | total: 0 residues |

Final Results (k = 9/23):

```
39.1%: nuclear
26.1%: cytoplasmic
13.0%: mitochondrial
  8.7%: vesicles of secretory system
  4.3%: vacuolar
  4.3%: peroxisomal
  4.3%: endoplasmic reticulum
```

>> prediction for CG56959-02 is nuc (k = 23)

A search of the NOV44a protein against the Geneseq database, a proprietary database that contains sequences published in patents and patent publication, yielded several homologous proteins shown in Table 44D.

TABLE 44D
Geneseq Results for NOV44a

| | | NOV44a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABJ04645 | Protein of NOVX 6 SEQ ID No 12 - | 1 . . . 523 | 522/523 (99%) | 0.0 |
| | Unidentified, 523 aa. [WO200246409- | 1 . . . 523 | 522/523 (99%) | |
| | A2, 13-JUN-2002] | | | |
| AAO19183 | Human neurotransmission-associated | 1 . . . 523 | 521/533 (97%) | 0.0 |
| | protein NTRAN6 - Homo sapiens, 533 | 1 . . . 533 | 522/533 (97%) | |
| | aa. [WO200266646-A2, 29-AUG-2002] | | | |
| ABP47935 | Human polypeptide SEQ ID NO 365 - | 78 . . . 501 | 296/424 (69%) | e−176 |
| | Homo sapiens, 461 aa. | 33 . . . 450 | 348/424 (81%) | |
| | [US2002042386-A1, 11-APR-2002] | | | |
| AAU19715 | Human novel extracellular matrix | 78 . . . 501 | 296/424 (69%) | e−176 |
| | protein, Seq ID No 365 - Homo | 33 . . . 450 | 348/424 (81%) | |
| | sapiens, 461 aa. [WO200155368-A1, | | | |
| | 02-AUG-2001] | | | |
| ABP69319 | Human polypeptide SEQ ID NO 1366 - | 84 . . . 501 | 291/418 (69%) | e−172 |
| | Homo sapiens, 425 aa. | 3 . . . 414 | 343/418 (81%) | |
| | [WO200270539-A2, 12-SEP-2002] | | | |

In a BLAST search of public sequence databases, the NOV44a protein was found to have homology to the proteins shown in the BLASTP data in Table 44E.

TABLE 44E
Public BLASTP Results for NOV44a

| | | NOV44a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q925B8 | Synaptotagmin 10 - Rattus | 1 . . . 523 | 487/523 (93%) | 0.0 |
| | norvegicus (Rat), 523 aa. | 1 . . . 523 | 502/523 (95%) | |
| Q9R0N4 | Synaptotagmin X (SytX) - Mus | 1 . . . 523 | 485/523 (92%) | 0.0 |
| | musculus (Mouse), 523 aa. | 1 . . . 523 | 501/523 (95%) | |
| O08625 | Synaptotagmin X (SytX) - Rattus | 1 . . . 498 | 462/498 (92%) | 0.0 |
| | norvegicus (Rat), 498 aa | 1 . . . 498 | 477/498 (95%) | |
| | (fragment). | | | |
| Q9R0N8 | Synaptotagmin VI - Mus musculus | 13 . . . 501 | 329/496 (66%) | 0.0 |
| | (Mouse), 511 aa. | 12 . . . 500 | 391/496 (78%) | |
| Q62746 | Synaptotagmin VI - Rattus | 13 . . . 501 | 329/496 (66%) | 0.0 |
| | norvegicus (Rat), 511 aa. | 12 . . . 500 | 389/496 (78%) | |

PFam analysis predicts that the NOV44a protein contains the domains shown in the Table 44F.

TABLE 44F
Domain Analysis of NOV44a

| | | Identities/ | |
|---|---|---|---|
| | | Similarities | |
| Pfam | NOV44a | for the | Expect |
| Domain | Match Region | Matched Region | Value |
| C2 | 248 . . . 334 | 44/89 (49%) | 6.2e−39 |
| | | 83/89 (93%) | |
| C2 | 380 . . . 468 | 38/90 (42%) | 1.6e−35 |
| | | 78/90 (87%) | |

### Example 45

The NOV45 clone was analyzed, and the nucleotide and encoded polypeptide sequences are shown in Table 45A.

TABLE 45A
NOV45 Sequence Analysis

| | | |
|---|---|---|
| NOV45a, CG57111-01 | SEQ ID NO: 1009 | 2855 bp |
| DNA Sequence | ORF Start: at 3 | ORF Stop: TGA at 2844 |

GAGGGAATGCGCGCAGCTCACAGGCCCTGGGAGTGAGCTGGTGCCCGGCGACCTGGCACCCGCGCCTG
GATATGGGGCGTCTACATCGTCCCAGGAGCAGCACCAGCTACAGGAACCTGCCGCATCTGTTTCTGTT
TTTCCTCTTCGTGGGACCCTTCAGCTGCCTCGGGAGTTACAGCCGGGCCACCGAGCTTCTGTACAGCC
TAAACGAGGGACTACCCGCGGGGGTGCTCATCGGCAGCCTGGCCGAGGACCTGCGGCTGCTGCCCAGG
TCTGCAGGGAGGCCGGACCCGCAGTCGCAGCTGCCAGAGCGCACCGGTGCTGAGTGGAACCCCCCTCT
CTCCTTCAGCCTGGCCTCCCGGGGACTGAGTGGCCAGTACGTGACCCTAGACAACCGCTCTGGGGAGC
TGCACACTTCAGCTCAGGAGATCGACAGGGAGGCCCTGTGTGTTGAAGGGGGTGGAGGGACTGCGTGG
AGCGGCAGCGTTTCCATCTCCTCCTCTCCTTCTGACTCTTGTCTTTTGCTGCTGGATGTGCTTGTCCT
GCCTCAGGAATACTTCAGGTTTGTGAAGGTGAAGATCGCCATCAGAGACATCAATGACAACGCCCCGC
AGTTCCCTGTTTCCCAGATCTCGGTGTGGGTCCCGGAAAATGCACCTGTAAACACCCGACTGGCCATA
GAGCATCCTGCTGTGGACCCAGATGTTGGCATTAATGGGGTTCAGACCTATCGCTTACTGGACTACCA
TGGTATGTTCACCCTGGACGTGGAGGAGAATGAGAATGGGGAGCGCACCCCCTACCTAATTGTCATGG
GTGCTTTGGACAGGGAAACCCAGGACCAGTATGTGAGCATCATCACAGCTGAGGATGGTGGGTCTCCA
CCACTTTTGGGCAGTGCCACTCTCACCATTGGCATCAGTGACATTAATGACAATTGCCCTCTCTTCAC
AGACTCACAAATCAATGTCACTGTGTATGGGAATGCTACAGTGGGCACCCCAATTGCAGCTGTCCAGG
CTGTGGATAAAGACTTGGGGACCAATGCTCAAATTACTTATTCTTACAGTCAGAAAGTTCCACAAGCA
TCTAAGGATTTATTTCACCTGGATGAAAACACTGGAGTCATTAAACTTTTCAGTAAGATTGGAGGAAG
TGTTCTGGAGTCCCACAAGCTCACCATCCTTGCTAATGGACCAGGCTGCATCCCTGCTGTAATCACTG
CTCTTGTGTCCATTATTAAAGTTATTTTCAGACCCCCTGAAATTGTCCCTCGTTACATAGCAAACGAG
ATAGATGGTGTTGTTTATCTGAAAGAACTGGAACCCGTTAACACTCCCATTGCGTTTTTCACCATAAG
AGATCCAGAAGGTAAATACAAGGTTAACTGCTACCTGGATGGTGAAGGGCCGTTTAGGTTATCACCTT
ACAAACCATACAATAATGAATATTTACTAGAGACCACAAAACCTATGGACTATGAGCTACAGCAGTTC
TATGAAGTAGCTGTGGTGGCTTGGAACTCTGAGGGATTTCATGTCAAAAGGGTCATTAAAGTGCAACT
TTTAGATGACAATGATAATGCTCCAATTTTCCTTCAACCCTTAATAGAACTAACCATCGAAGAGAACA
ACTCACCCAATGCCTTTTTGACTAAGCTGTATGCTACAGATGCCGACAGCGAGGAGAGAGGCCAAGTT
TCATATTTTCTGGGACCTGATGCTCCATCATATTTTTCCTTAGACAGTGTCACAGGAATTCTGACAGT
TTCTACTCAGCTGGACCGAGAAGAGAAAGAAAAGTACAGATACACTGTCAGAGCTGTTGACTGTGGGA
AGCCACCCAGAGAATCAGTAGCCACTGTGGCCCTCACAGTGTTGGATAAAAATGACAACAGTCCTCGG
TTTATCAACAAGGACTTCAGCTTTTTTGTGCCTGAAAACTTTCCAGGCTATGGTGAGATTGGAGTAAT
TAGTGTAACAGATGCTGACGCTGGACGAAATGGATGGGTCGCCCTCTCTGTGGTGAACCAGAGTGATA
TTTTTGTCATAGATACAGGAAAGGGTATGCTGAGGGCTAAAGTCTCTTTGGACAGAGAGCAGCAAAGC
TCCTATACTTTGTGGGTTGAAGCTGTTGATGGGGGTGAGCCTGCCCTCTCCTCTACAGCAAAAATCAC
AATTCTCCTTCTAGATATCAATGACAACCCTCCTCTTGTTTTGTTTCCTCAGTCTAATATGTCTTATC
TGTTAGTACTGCCTTCTACTCTGCCAGGCTCCCCGGTTACAGAAGTCTATGCTGTCGACAAAGACACA
GGCATGAATGCTGTCATAGCTTACAGCATCATAGGGAGAAGAGGTCCTAGGCCTGAGTCCTTCAGGAT
TGACCCTAAAACTGGCAACATTACTTTGGAAGAGGCATTGCTGCAGACAGATTATGGGCTCCATCGCT
TACTGGTGAAAGTGAGTGATCATGGTTATCCCGAGCCTCTCCACTCCACAGTCATGGTGAACCTATTT
GTCAATGACACTGTCAGTAATGAGAGTTACATTGAGAGTCTTTTAAGAAAAGAACCAGAGATTAATAT
AGAGGAGAAAGAACCACAAATCTCAATAGAACCGACTCATAGGAAGGTAGAATCTGTGTCTTGTATGC
CCACCTTAGTAGCTCTGTCTGTAATAAGCTTGGGTTCCATCACACTGGTCACAGGGATGGGCATATAC
ATCTGTTTAAGGAAAGGGGAAAAGCATCCCAGGGAAGATGAAAATTTGGAAGTACAGATTCCACTGAA
AGGAAAAATTGACTTGCATATGCGAGAGAGAAAGCCAATGGATATTTCTAATATTTGATATTTCATG

| | | | |
|---|---|---|---|
| NOV45a, CG57111-01 | SEQ ID NO: 1010 | 947 aa | MW at 104405.1kD |
| Protein Sequence | | | |

GNARSSQALGVSWCPATWHPRLDMGRLHRPRSSTSYRNLPHLFLFFLFVGPFSCLGSYSRATELLYSL
NEGLPAGVLIGSLAEDLRLLPRSAGRPDPQSQLPERTGAEWNPPLSFSLASRGLSGQYVTLDNRSGEL
HTSAQEIDREALCVEGGGGTAWSGSVSISSSPSDSCLLLLDVLVLPQEYFRFVKVKIAIRDINDNAPQ
FPVSQISVWVPENAPVNTRLAIEHPAVDPDVGINGVQTYRLLDYHGMFTLDVEENENGERTPYLIVMG
ALDRETQDQYVSIITAEDGGSPPLLGSATLTIGISDINDNCPLFTDSQINVTVYGNATVGTPIAAVQA
VDKDLGTNAQITYSYSQKVPQASKDLFHLDENTGVIKLFSKIGGSVLESHKLTILANGPGCIPAVITA
LVSIIKVIFRPPEIVPRYIANEIDGVVYLKELEPVNTPIAFFTIRDPEGKYKVNCYLDGEGPFRLSPY
KPYNNEYLLETTKPMDYELQQFYEVAVVAWNSEGFHVKRVIKVQLLDDNDNAPIFLQPLIELTIEENN
SPNAFLTKLYATDADSEERGQVSYFLGPDAPSYFSLDSVTGILTVSTQLDREEKEKYRYTVRAVDCGK
PPRESVATVALTVLDKNDNSPRFINKDFSFFVPENFPGYGEIGVISVTDADAGRNGWVALSVVNQSDI
FVIDTGKGMLRAKVSLDREQQSSYTLWVEAVDGGEPALSSTAKITILLLDINDNPPLVLFPQSNMSYL
LVLPSTLPGSPVTEVYAVDKDTGNNAVIAYSIIGRRGPRPESFRIDPKTGNITLEEALLQTDYGLHRL
LVKVSDHGYPEPLHSTVMVNLFVNDTVSNESYIESLLRKEPEINIEEKEPQISIEPTHRKVESVSCMP
TLVALSVISLGSITLVTGMGIYICLRKGEKHPREDENLEVQIPLKGKIDLHMRERKPMDISNI

| | | |
|---|---|---|
| NOV45b, 277726328 | SEQ ID NO: 1011 | 2854 bp |
| DNA Sequence | ORF Start: at 2 | ORF Stop: end of sequence |

GGGATCCGGGAATGCGCGCAGCTCACAGGCCCTGGGAGTGAGCTGGTGCCCGGCGACCTGGCACCCGC
GCCTGGATATGGGGCGTCTACATCGTCCCAGGAGCAGCACCAGCTACAGGAACCTGCCGCATCTGTTT
CTGTTTTTCCTCTTCGTGGGACCCTTCAGCTGCCTCGGGAGTTACAGCCGGGCCACCGAGCTTCTGTA
CAGCCTAAACGAGGGACTACCCGCGGGGGTGCTCATCGGCAGCCTGGCCGAGGACCTGCGGCTGCTGC
CCAGGTCTGCAGGGAGGCCGGACCCGCAGTCGCAGCTGCCAGAGCGCACCGGTGCTGAGTGGAACCCC
CCTCTCTCCTTCAGCCTGGCCTCCCGGGGACTGAGTGGCCAGTACGTGACCCTAGACAACCGCTCTGG
GGAGCTGCACACTTCAGCTCAGGAGATCGACAGGGAGGCCCTGTGTGTTGAAGGGGGTGGAGGGACTG
CGTGGAGCGGCAGCGTTTCCATCTCCTCCTCTCCTTCTGACTCTTGTCTTTTGCTGCTGGATGTGCTT
GTCCTGCCTCAGGAATACTTCAGGTTTGTGAAGGTGAAGATCGCCATCAGAGACATCAATGACAACGC
CCCGCAGTTCCCTGTTTCCCAGATCTCGGTGTGGGTCCCGGAAAATGCACCTGTAAACACCCGACTGG
CCATAGAGCATCCTGCTGTGGACCCAGATGTAGGCATTAATGGGGTACAGACCTATCGCTTACTGGAC
TACCATGGTATGTTCACCCTGGACGTGGAGGAGAATGAGAATGGGGAGCGCACCCCCTACCTAATTGT
CATGGGTGCTTTGGACAGGGAAACCCAGGACCAGTATGTGAGCATCATCATAGCTGAGGATGGTGGGT
CTCCACCACTTTTGGGCAGTGCCACTCTCACCATTGGCATCAGTGACATTAATGACAATTGCCCTCTC
TTCACAGACTCACAAATCAATGTCACTGTGTATGGGAATGCTACAGTGGGCACCCCAATTGCAGCTGT
CCAGGCTGTGGATAAAGACTTGGGGACCAATGCTCAAATTACTTATTCTTACAGTCAGAAAGTTCCAC
AAGCATCTAAGGATTTATTTCACCTGGATGAAAACACTGGAGTCATTAAACTTTTCAGTAAGATTGGA
GGAAGTGTTCTGGAGTCCCACAAGCTCACCATCCTTGCTAATGGACCAGGCTGCATCCCTGCTGTAAT
CACTGCTCTTGTGTCCATTATTAAAGTTATTTTCAGACCCCCTGAAATTGTCCCTCGTTACATAGCAA
ACGAGATAGATGGTGTTGTTTATCTGAAAGAACTGGAACCCGTTAACACTCCCATTGCGTTTTTCACC
ATAAGAGATCCAGAAGGTAAATACAAGGTTAACTGCTACCTGGATGGTGAAGGGCCGTTTAGGTTATC
ACCTTACAAACCATACAATAATGAATATTTACTAGAGACCACAAAACCTATGGACTATGAGCTACAGC
AGTTCTATGAAGTAGCTGTGGTGGCTTGGAACTCTGAGGGATTTCATGTCAAAAGGGTCATTAAAGTG
CAACTTTTAGATGACAATGATAATGCTCCAATTTTCCTTCAACCCTTAATAGAACTAACCATCGAAGA
GAACAACTCACCCAATGCCTTTTTGACTAAGCTGTATGCTACAGATGCCGACAGCGAGGAGAGAGGCC
AAGTTTCATATTTTCTGGGACCTGATGCTCCATCATATTTTTCCTTAGACAGTGTCACAGGAATTCTG
ACAGTTTCTACTCAGCTGGACCGAGAAGAGAAAGAAAAGTACAGATACACTGTCAGAGCTGTTGACTG
TGGGAAGCCACCCAGAGAATCAGTAGCCACTGTGGCCCTCACAGTGTTGGATAAAAATGACAACAGTC
CTCGGTTTATCAACAAGGACTTCAGCTTTTTTGTGCCTGAAAACTTTCCAGGCTATGGTGAGATTGGA
GTAATTAGTGTAACAGATGCTGACGCTGGACGAAATGGATGGGTCGCCCTCTCTGTGGTGAACCAGAG
TGGTATTTTTGTCATAGATACAGGAAAGGGTATGCTGAGGGCTAAAGTCTCTTTGGACAGAGAGCAGC
AAAGCTCCTATACTTTGTGGGTTGAAGCTGTTGATGGGGGTGAGCCTGCCCTCTCCTCTACAGCAAAA
ATCACAATTCTCCTTCTAGATATCAATGACAACCCTCCTCTTGTTTTGTTTCCTCAGTCTAATATGTC
TTATCTGTTAGTACTGCCTTCTACTCTGCCAGGCTCCCCGGTTACAGAAGTCTATGCTGTCGACAAAG
ACACAGGCATGAATGCTGTCATAGCTTACAGCATCATAGGGAGAAGAGGTCCTAGGCCTGAGTCCTTC
AGGATTGACCCTAAAACTGGCAACATTACTTTGGAAGAGGCATTGCTGCAGACAGATTATGGGCTCCA
TCGCTTACTGGTGAAAGTGAGTGATCATGGTTATCCCGAGCCTCTCCACTCCACAGTCATGGTGAACC
TATTTGTCAATGACACTGTCAGTAATGAGAGTTACATTGAGAGTCTTTTAAGAAAAGAACCAGAGATT
AATATAGAGGAGAAAGAACCACAAATCTCAATAGAACCGACTCATAGGAAGGTAGAATCTGTGTCTTG
TATGCCCACCTTAGTAGCTCTGTCTGTAATAAGCTTGGGTTCCATCACACTGGTCACAGGGATGGGCA
TATACATCTGTTTAAGGAAAGGGGAAAAGCATCCCAGGGAAGATGAAAATTTGGAAGTACAGATTCCA
CTGAAAGGAAAAATTGACTTGCATATGCGAGAGAGAAAGCCAATGGATATTTCTAATATTCTCGAG

| | | | |
|---|---|---|---|
| NOV45b, 277726328 | SEQ ID NO: 1012 | 951 aa | MW at 104745.6kD |
| Protein Sequence | | | |

GSGNARSSQALGVSWCPATWHPRLDMGRLHRPRSSTSYRNLPHLFLFFLFVGPFSCLGSYSRATELLY
SLNEGLPAGVLIGSLAEDLRLLPRSAGRPDPQSQLPERTGAEWNPPLSFSLASRGLSGQYVTLDNRSG
ELHTSAQEIDREALCVEGGGGTAWSGSVSISSSPSDSCLLLLDVLVLPQEYFRFVKVKIAIRDINDNA
PQFPVSQISVWVPENAPVNTRLAIEHPAVDPDVGINGVQTYRLLDYHGMFTLDVEENENGERTPYLIV
MGALDRETQDQYVSIIIAEDGGSPPLLGSATLTIGISDINDNCPLFTDSQINVTVYGNATVGTPIAAV
QAVDKDLGTNAQITYSYSQKVPQASKDLFHLDENTGVIKLFSKIGGSVLESHKLTILANGPGCIPAVI
TALVSIIKVIFRPPEIVPRYIANEIDGVVYLKELEPVNTPIAFFTIRDPEGKYKVNCYLDGEGPFRLS
PYKPYNNEYLLETTKPMDYELQQFYEVAVVAWNSEGFHVKRVIKVQLLDDNDNAPIFLQPLIELTIEE
NNSPNAFLTKLYATDADSEERGQVSYFLGPDAPSYFSLDSVTGILTVSTQLDREEKEKYRYTVRAVDC
GKPPRESVATVALTVLDKNDNSPRFINKDFSFFVPENFPGYGEIGVISVTDADAGRNGWVALSVVNQS
GIFVIDTGKGMLRAKVSLDREQQSSYTLWVEAVDGGEPALSSTAKITILLLDINDNPPLVLFPQSNMS
YLLVLPSTLPGSPVTEVYAVDKDTGMNAVIAYSIIGRRGPRPESFRIDPKTGNITLEEALLQTDYGLH
RLLVKVSDHGYPEPLHSTVMVNLFVNDTVSNESYIESLLRKEPEINIEEKEPQISIEPTHRKVESVSC
MPTLVALSVISLGSITLVTGMGIYICLRKGEKHPREDENLEVQIPLKGKIDLHMRERKPMDISNILE

| | | |
|---|---|---|
| NOV45c, CG57111-02 | SEQ ID NO: 1013 | 1020 bp |
| DNA Sequence | ORF Start: at 1 | ORF Stop: end of sequence |

AGTTACAGCCGGGCCACCGAGCTTCTGTACAGCCTAAACGAGGGACTACCCGCGGGGGTGCTCATCGG
CAGCCTGGCCGAGGACCTGCGGCTGCTGCCCAGGTCTGCAGGGAGGCCGGACCCGCAGTCGCAGCTGC
CAGAGCGCACCGGTGCTGAGTGGAACCCCCCTCTCTCCTTCAGCCTGGCCTCCCGGGGACTGAGTGGC
CAGTACGTGACCCTAGACAACCGCTCTGGGGAGCTGCACACTTCAGCTCAGGAGATCGACAGGGAGGC
CCTGTGTGTTGAAGGGGGTGGAGGGACTGCGTGGAGCGGCAGCGTTTCCATCTCCTCCTCTCCTTCTG
ACTCTTGTCTTTTGCTGCTGGATGTGCTTGTCCTGCCTCAGGAATACTTCAGGTTTGTGAAGGTGAAG
ATCGCCATCAGAGACATCAATGACAACGCCCCGCAGTTCCCTGTTTCCCAGATCTCGGTGTGGGTCCC
GGAAAATGCACCTGTAAACACCCGACTGGCCATAGAGCATCCTGCTGTGGACCCAGATGTAGGCATTA
ATGGGGTGCAGACCTATCGCTTACTGGACTACCATGGTATGTTCACCCTGGACGTGGAGGAGAATGAG
AATGGGGAGCGCACCCCCTACCTAATTGTCATGGGTGCTTTGGACAGGGAAACCCAGGACCAGTATGT
GAGCATCATCATAGCTGAGGATGGTGGGTCTCCACCACTTTTGGGCAGTGCCACTCTCACCATTGGCA
TCAGTGACATTAATGACAATTGCCCTCTCTTCACAGACTCACAAATCAATGTCACTGTGTATGGGAAT
GCTACAGTGGGCACCCCAATTGCAGCTGTCCAGGCTGTGGATAAAGACTTGGGGACCAATGCTCAAAT
TACTTATTCTTACAGTCAGAAAGTTCCACAAGCATCTAAGGATTTATTTCACCTGGATGAAAACACTG
GAGTCATTAAACTTTTCAGTAAGATTGGAGGAAGTGTTCTGGAGTCCCACAAGCTCACCATCCTTGCT

| | | | |
|---|---|---|---|
| NOV45c, CG57111-02 | SEQ ID NO: 1014 | 340 aa | MW at 36598.6kD |
| Protein Sequence | | | |

SYSRATELLYSLNEGLPAGVLIGSLAEDLRLLPRSAGRPDPQSQLPERTGAEWNPPLSFSLASRGLSG
QYVTLDNRSGELHTSAQEIDREALCVEGGGGTAWSGSVSISSSPSDSCLLLLDVLVLPQEYFRFVKVK
IAIRDINDNAPQFPVSQISVWVPENAPVNTRLAIEHPAVDPDVGINGVQTYRLLDYHGMFTLDVEENE
NGERTPYLIVMGALDRETQDQYVSIIIAEDGGSPPLLGSATLTIGISDINDNCPLFTDSQINVTVYGN
ATVGTPIAAVQAVDKDLGTNAQITYSYSQKVPQASKDLFHLDENTGVIKLFSKIGGSVLESHKLTILA

| | | |
|---|---|---|
| NOV45d, CG57111-03 | SEQ ID NO: 1015 | 1032 bp |
| DNA Sequence | ORF Start: at 7 | ORF Stop: at 1027 |

GGATCCAGTTACAGCCGGGCCACCGAGCTTCTGTACAGCCTAAACGAGGGACTACCCGCGGGGGTGCT
CATCGGCAGCCTGGCCGAGGACCTGCGGCTGCTGCCCAGGTCTGCAGGGAGGCCGGACCCGCAGTCGC
AGCTGCCAGAGCGCACCGGTGCTGAGTGGAACCCCCCTCTCTCCTTCAGCCTGGCCTCCCGGGGACTG
AGTGGCCAGTACGTGACCCTAGACAACCGCTCTGGGGAGCTGCACACTTCAGCTCAGGAGATCGACAG
GGAGGCCCTGTGTGTTGAAGGGGGTGGAGGGACTGCGTGGAGCGGCAGCGTTTCCATCTCCTCCTCTC
CTTCTGACTCTTGTCTTTTGCTGCTGGATGTGCTTGTCCTGCCTCAGGAATACTTCAGGTTTGTGAAG
GTGAAGATCGCCATCAGAGACATCAATGACAACGCCCCGCAGTTCCCTGTTTCCCAGATCTCGGTGTG
GGTCCCGGAAAATGCACCTGTAAACACCCGACTGGCCATAGAGCATCCTGCTGTGGACCCAGATGTAG
GCATTAATGGGGTGCAGACCTATCGCTTACTGGACTACCATGGTATGTTCACCCTGGACGTGGAGGAG
AATGAGAATGGGGAGCGCACCCCCTACCTAATTGTCATGGGTGCTTTGGACAGGGAAACCCAGGACCA
GTATGTGAGCATCATCATAGCTGAGGATGGTGGGTCTCCACCACTTTTGGGCAGTGCCACTCTCACCA
TTGGCATCAGTGACATTAATGACAATTGCCCTCTCTTCACAGACTCACAAATCAATGTCACTGTGTAT
GGGAATGCTACAGTGGGCACCCCAATTGCAGCTGTCCAGGCTGTGGATAAAGACTTGGGGACCAATGC
TCAAATTACTTATTCTTACAGTCAGAAAGTTCCACAAGCATCTAAGGATTTATTTCACCTGGATGAAA
ACACTGGAGTCATTAAACTTTTCAGTAAGATTGGAGGAAGTGTTCTGGAGTCCCACAAGCTCACCATC
CTTGCTCTCGAG

| | | | |
|---|---|---|---|
| NOV45d, CG57111-03 | SEQ ID NO: 1016 | 340 aa | MW at 36598.6kD |
| Protein Sequence | | | |

SYSRATELLYSLNEGLPAGVLIGSLAEDLRLLPRSAGRPDPQSQLPERTGAEWNPPLSFSLASRGLSG
QYVTLDNRSGELHTSAQEIDREALCVEGGGGTAWSGSVSISSSPSDSCLLLLDVLVLPQEYFRFVKVK
IAIRDINDNAPQFPVSQISVWVPENAPVNTRLAIEHPAVDPDVGINGVQTYRLLDYHGMFTLDVEENE
NGERTPYLIVMGALDRETQDQYVSIIIAEDGGSPPLLGSATLTIGISDINDNCPLFTDSQINVTVYGN
ATVGTPIAAVQAVDKDLGTNAQITYSYSQKVPQASKDLFHLDENTGVIKLFSKIGGSVLESHKLTILA

| | | |
|---|---|---|
| NOV45e, CG57111-04 | SEQ ID NO: 1017 | 1390 bp |
| DNA Sequence | ORF Start: at 8 | ORF Stop: at 1385 |

GGGATCCCCTGAAATTGTCCCTCGTTACATAGCAAACGAGATAGATGGTGTTGTTTATCTGAAAGAAC
TGGAACCCGTTAACACTCCCATTGCGTTTTTCACCATAAGAGATCCAGAAGGTAAATACAAGGTTAAC
TGCTACCTGGATGGTGAAGGGCCGTTTAGGTTATCACCTTACAAACCATACAATAATGAATATTTACT
AGAGACCACAAAACCTATGGACTATGAGCTACAGCAGTTCTATGAAGTAGCTGTGGTGGCTTGGAACT
CTGAGGGATTTCATGTCAAAAGGGTCATTAAAGTGCAACTTTTAGATGACAATGATAATGCTCCAATT
TTCCTTCAACCCTTAATAGAACTAACCATCGAAGAGAACAACTCACCCAATGCCTTTTTGACTAAGCT
GTATGCTACAGATGCCGACAGCGAGGAGAGAGGCCAAGTTTCATATTTTCTGGGACCTGATGCTCCAT
CATATTTTTCCTTAGACAGTGTCACAGGAATTCTGACAGTTTCTACTCAGCTGGACCGAGAAGAGAAA
GAAAAGTACAGATACACTGTCAGAGCTGTTGACTGTGGGAAGCCACCCAGAGAATCAGTAGCCACTGT
GGCCCTCACAGTGTTGGATAAAAATGACAACAGTCCTCGGTTTATCAACAAGGACTTCAGCTTTTTTG
TGCCTGAAAACTTTCCAGGCTATGGTGAGATTGGAGTAATTAGTGTAACAGATGCTGACGCTGGACGA
AATGGATGGGTCGCCCTCTCTGTGGTGAACCAGAGTGATATTTTTGTCATAGATACAGGAAAGGGTAT
GCTGAGGGCTAAAGTCTCTTTGGACAGAGAGCAGCAAAGCTCCTATACTTTGTGGGTTGAAGCTGTTG
ATGGGGGTGAGCCTGCCCTCTCCTCTACAGCAAAAATCACAATTCTCCTTCTAGATATCAATGACAAC
CCTCCTCTTGTTTTGTTTCCTCAGTCTAATATGTCTTATCTGTTAGTACTGCCTTCTACTCTGCCAGG
CTCCCCGGTTACAGAAGTCTATGCTGTCGACAAAGACACAGGCATGAATGCTGTCATAGCTTACAGCA
TCATAGGGAGAAGAGGTCCTAGGCCTGAGTCCTTCAGGATTGACCCTAAAACTGGCAACATTACTTTG
GAAGAGGCATTGCTGCAGACAGATTATGGGCTCCATCGCTTACTGGTGAAAGTGAGTGATCATGGTTA
TCCCGAGCCTCTCCACTCCACAGTCATGGTGAACCTATTTGTCAATGACACTGTCAGTAATGAGAGTT
ACATTGAGAGTCTTTTAAGAAAAGAACCAGAGATTAATATAGAGGAGAAAGAACCACAAATCTCAATA
GAACCGACTCATAGGAAGGTAGAACTCGAG

| | | | |
|---|---|---|---|
| NOV45e, CG57111-04 | SEQ ID NO: 1018 | 459 aa | MW at 51489.5kD |
| Protein Sequence | | | |

PEIVPRYIANEIDGVVYLKELEPVNTPIAFFTIRDPEGKYKVNCYLDGEGPFRLSPYKPYNNEYLLET
TKPMDYELQQFYEVAVVAWNSEGFHVKRVIKVQLLDDNDNAPIFLQPLIELTIEENNSPNAFLTKLYA
TDADSEERGQVSYFLGPDAPSYFSLDSVTGILTVSTQLDREEKEKYRYTVRAVDCGKPPRESVATVAL
TVLDKNDNSPRFINKDFSFFVPENFPGYGEIGVISVTDADAGRNGWVALSVVNQSDIFVIDTGKGMLR
AKVSLDREQQSSYTLWVEAVDGGEPALSSTAKITILLLDINDNPPLVLFPQSNMSYLLVLPSTLPGSP
VTEVYAVDKDTGMNAVIAYSIIGRRGPRPESFRIDPKTGNITLEEALLQTDYGLHRLLVKVSDHGYPE
PLHSTVMVNLFVNDTVSNESYIESLLRKEPEINIEEKEPQISIEPTHRKVE

A ClustalW comparison of the above protein sequences yields the following sequence alignment shown in Table 45B.

TABLE 45B
Comparison of the NOV45 protein sequences.

| | |
|---|---|
| NOV45a | --GNARSSQALGVSWCPATWHPRLDMGRLHRPRSSTSYRNLPHLFLFFLFVGPFSCLGSY |
| NOV45b | GSGNARSSQALGVSWCPATWHPRLDMGRLHRPRSSTSYRNLPHLFLFFLFVGPFSCLGSY |
| NOV45c | ----------------------------------------------------------SY |
| NOV45d | ----------------------------------------------------------SY |
| NOV45e | -------------------------------------------PEIVPRYIANEIDGVVY |
| NOV45a | SRATELLYSLNEGLPAGVLIGSLAEDLRLLPRSAGRPDPQSQLPERTGAEWNPPLSFSLA |
| NOV45b | SRATELLYSLNEGLPAGVLIGSLAEDLRLLPRSAGRPDPQSQLPERTGAEWNPPLSFSLA |
| NOV45c | SRATELLYSLNEGLPAGVLIGSLAEDLRLLPRSAGRPDPQSQLPERTGAEWNPPLSFSLA |
| NOV45d | SRATELLYSLNEGLPAGVLIGSLAEDLRLLPRSAGRPDPQSQLPERTGAEWNPPLSFSLA |
| NOV45e | LKELEPVNTPIAFFTIRDPEGKYKVNCYLDGEGPFRLSPYKPYNNEYLLETTKPMDYELQ |
| NOV45a | SRGLSGQYVTLDN---------------------------------RSGELHTSAQEIDR |
| NOV45b | SRGLSGQYVTLDN---------------------------------RSGELHTSAQEIDR |
| NOV45c | SRGLSGQYVTLDN---------------------------------RSGELHTSAQEIDR |
| NOV45d | SRGLSGQYVTLDN---------------------------------RSGELHTSAQEIDR |
| NOV45e | QFYEVAVVAWNSEGFHVKRVIKVQLLDDNDNAPIFLQPLIELTIEENNSPNAFLTKLYAT |
| NOV45a | EALCVEGGGGTAWSGSVSISSSPSDSCLLLLDVLVLPQEYFR------------------ |
| NOV45b | EALCVEGGGGTAWSGSVSISSSPSDSCLLLLDVLVLPQEYFR------------------ |
| NOV45c | EALCVEGGGGTAWSGSVSISSSPSDSCLLLLDVLVLPQEYFR------------------ |
| NOV45d | EALCVEGGGGTAWSGSVSISSSPSDSCLLLLDVLVLPQEYFR------------------ |
| NOV45e | DADSEERGQVSYFLGPDAPSYFSLDSVTGILTVSTQLDREEKEKYRYTVRAVDCGKPPRE |
| NOV45a | -FVKVKIAIRDINDNAPQFPVSQISVWVPENAPVNTRLAIEHPAVDPDVGINGVQTYRLL |
| NOV45b | -FVKVKIAIRDINDNAPQFPVSQISVWVPENAPVNTRLAIEHPAVDPDVGINGVQTYRLL |
| NOV45c | -FVKVKIAIRDINDNAPQFPVSQISVWVPENAPVNTRLAIEHPAVDPDVGINGVQTYRLL |
| NOV45d | -FVKVKIAIRDINDNAPQFPVSQISVWVPENAPVNTRLATEHPAVDPDVGINGVQTYRLL |
| NOV45e | SVATVALTVLDKNDNSPRFINKDFSFFVPENFPGYGEIGVIS-VTDADAGRNGWVALSVV |
| NOV45a | DYHGMFTLDVEENENGERTPYLIVMGALDRETQDQYVSIITAEDGGSPPLLGSATLTIGI |
| NOV45b | DYHGMFTLDVEENENGERTPYLIVMGALDRETQDQYVSIIIAEDGGSPPLLGSATLTIGI |
| NOV45c | DYHGMFTLDVEENENGERTPYLIVMGALDRETQDQYVSIIIAEDGGSPPLLGSATLTIGI |
| NOV45d | DYHGMFTLDVEENENGERTPYLIVMGALDRETQDQYVSIIIAEDGGSPPLLGSATLTIGI |
| NOV45e | NQSDIFVIDTGKG-------MLRAKVSLDREQQSSYTLWVEAVDGGEPALSSTAKITILL |
| NOV45a | SDINDNCPLFTDSQIN---VTVYGNATVGTPIAAVQAVDKDLGTNAQITYSYSQKVPQAS |
| NOV45b | SDINDNCPLFTDSQIN---VTVYGNATVGTPIAAVQAVDKDLGTNAQITYSYSQKVPQAS |
| NOV45c | SDINDNCPLFTDSQIN---VTVYGNATVGTPIAAVQAVDKDLGTNAQITYSYSQKVPQAS |
| NOV45d | SDINDNCPLFTDSQIN---VTVYGNATVGTPIAAVQAVDKDLGTNAQITYSYSQKVPQAS |
| NOV45e | LDINDNPPLVLFPQSNMSYLLVLPSTLPGSPVTEVYAVDKDTGMNAVIAYSIIGRR-GPR |
| NOV45a | KDLFHLDENTGVIKLFSKIGGSVLESHKLTILANGPGCIPAVITALVSIIKVIFRPPEIV |
| NOV45b | KDLFHLDENTGVIKLFSKIGGSVLESHKLTILANGPGCIPAVITALVSIIKVIFRPPEIV |
| NOV45c | KDLFHLDENTGVIKLFSKIGGSVLESHKLTILA--------------------------- |
| NOV45d | KDLFHLDENTGVIKLFSKIGGSVLESHKLTILA--------------------------- |
| NOV45e | PESFRIDPKTGNITLEEALLQTDYGLHRLLVKVSDHGYPEPLHSTVMVNLFVNDTVSNES |
| NOV45a | PRYIANEIDGVVYLKELEPVNTPIAFFTIRDPEGKYKVNCYLDGEGPFRLSPYKPYNNEY |
| NOV45b | PRYIANEIDGVVYLKELEPVNTPIAFFTIRDPEGKYKVNCYLDGEGPFRLSPYKPYNNEY |
| NOV45c | ------------------------------------------------------------ |
| NOV45d | ------------------------------------------------------------ |
| NOV45e | YIESLLRKEPEINIEEKEPQISIEPTHRKVE----------------------------- |
| NOV45a | LLETTKPMDYELQQFYEVAVVAWNSEGFHVKRVIKVQLLDDNDNAPIFLQPLIELTIEEN |
| NOV45b | LLETTKPMDYELQQFYEVAVVAWNSEGFHVKRVIKVQLLDDNDNAPIFLQPLIELTIEEN |
| NOV45c | ------------------------------------------------------------ |
| NOV45d | ------------------------------------------------------------ |
| NOV45e | ------------------------------------------------------------ |
| NOV45a | NSPNAFLTKLYATDADSEERGQVSYFLGPDAPSYFSLDSVTGILTVSTQLDREEKEKYRY |
| NOV45b | NSPNAFLTKLYATDADSEERGQVSYFLGPDAPSYFSLDSVTGILTVSTQLDREEKEKYRY |
| NOV45c | ------------------------------------------------------------ |
| NOV45d | ------------------------------------------------------------ |
| NOV45e | ------------------------------------------------------------ |
| NOV45a | TVRAVDCGKPPRESVATVALTVLDKNDNSPRFINKDFSFFVPENFPGYGEIGVISVTDAD |
| NOV45b | TVRAVDCGKPPRESVATVALTVLDKNDNSPRFINKDFSFFVPENFPGYGEIGVISVTDAD |
| NOV45c | ------------------------------------------------------------ |
| NOV45d | ------------------------------------------------------------ |
| NOV45e | ------------------------------------------------------------ |
| NOV45a | AGRNGWVALSVVNQSDIFVIDTGKGMLRAKVSLDREQQSSYTLWVEAVDGGEPALSSTAK |
| NOV45b | AGRNGWVALSVVNQSGIFVIDTGKGMLRAKVSLDREQQSSYTLWVEAVDGGEPALSSTAK |
| NOV45c | ------------------------------------------------------------ |
| NOV45d | ------------------------------------------------------------ |
| NOV45e | ------------------------------------------------------------ |
| NOV45a | ITILLLDINDNPPLVLFPQSNMSYLLVLPSTLPGSPVTEVYAVDKDTGMNAVIAYSIIGR |
| NOV45b | ITILLLDINDNPPLVLFPQSNMSYLLVLPSTLPGSPVTEVYAVDKDTGMNAVIAYSIIGR |
| NOV45c | ------------------------------------------------------------ |
| NOV45d | ------------------------------------------------------------ |
| NOV45e | ------------------------------------------------------------ |
| NOV45a | RGPRPESFRIDPKTGNITLEEALLQTDYGLHRLLVKVSDHGYPEPLHSTVMVNLFVNDTV |
| NOV45b | RGPRPESFRIDPKTGNITLEEALLQTDYGLHRLLVKVSDHGYPEPLHSTVMVNLFVNDTV |
| NOV45c | ------------------------------------------------------------ |
| NOV45d | ------------------------------------------------------------ |
| NOV45e | ------------------------------------------------------------ |
| NOV45a | SNESYIESLLRKEPEINIEEKEPQISIEPTHRKVESVSCMPTLVALSVISLGSITLVTGM |
| NOV45b | SNESYIESLLRKEPEINIEEKEPQISIEPTHRKVESVSCMPTLVALSVISLGSITLVTGM |
| NOV45c | ------------------------------------------------------------ |
| NOV45d | ------------------------------------------------------------ |
| NOV45e | ------------------------------------------------------------ |
| NOV45a | GIYICLRKGEKHPREDENLEVQIPLKGKIDLHMRERKPMDISNI-- |
| NOV45b | GIYICLRKGEKHPREDENLEVQIPLKGKIDLHMRERKPMDISNILE |
| NOV45c | ---------------------------------------------- |
| NOV45d | ---------------------------------------------- |
| NOV45e | ---------------------------------------------- |
| NOV45a | (SEQ ID NO: 1010) |
| NOV45b | (SEQ ID NO: 1012) |
| NOV45c | (SEQ ID NO: 1014) |
| NOV45d | (SEQ ID NO: 1016) |
| NOV45e | (SEQ ID NO: 1018) |

Further analysis of the NOV45a protein yielded the following properties shown in Table 45C.

TABLE 45C
Protein Sequence Properties NOV45a

| | |
|---|---|
| SignalP analysis: | Cleavage site between residues 57 and 58 |
| PSORT II analysis: | |

| PSG: | a new signal peptide prediction method | |
|---|---|---|
| | N-region: | length 4; pos. chg 1; neg. chg 0 |
| | H-region: | length 16; peak value 6.44 |
| | PSG score: | 2.04 |

| | |
|---|---|
| GvH: | von Heijne's method for signal seq. recognition |
| | GvH score (threshold: −2.1): −0.42 |
| | possible cleavage site: between 56 and 57 |

>>> Seems to have a cleavable signal peptide (1 to 56)

| ALOM: | Klein et al's method for TM region allocation | | |
|---|---|---|---|
| | Init position for calculation: 57 | | |
| | Tentative number of TMS(s) for the threshold 0.5: 2 | | |
| | INTEGRAL | Likelihood = | Transmembrane 401-417 |
| | | −7.38 | |
| | INTEGRAL | Likelihood = | Transmembrane 886-902 |
| | | −3.77 | |

| | |
|---|---|
| PERIPHERAL | Likelihood = 2.01 (at 173) |

| | |
|---|---|
| | ALOM score: −7.38 (number of TMSs: 2) |
| MTOP: | Prediction of membrane topology (Hartmann et al.) |
| | Center position for calculation: 28 |
| | Charge difference: 0.5 C(3.5)-N(3.0) |
| | C > N: C-terminal side will be inside |

>>>Caution: Inconsistent mtop result with signal peptide
>>> membrane topology: type 3b

| MITDISC: | discrimination of mitochondrial targeting seq | | | |
|---|---|---|---|---|
| | R content: | 2 | Hyd Moment(75): | 3.38 |
| | Hyd Moment(95): | 4.41 | G content: | 2 |
| | D/E content: | 1 | S/T content: | 4 |

| | |
|---|---|
| | Score: −3.49 |
| Gavel: | prediction of cleavage sites for mitochondrial preseq |
| | R-2 motif at 70 SRA\|TE |

NUCDISC: discrimination of nuclear localization signals

| | |
|---|---|
| | pat4: none |
| | pat7: none |
| | bipartite: none |
| | content of basic residues: 8.6% |
| | NLS Score: −0.47 |
| KDEL: | ER retention motif in the C-terminus: none |

ER Membrane Retention Signals:

| | |
|---|---|
| | XXRR-like motif in the N-terminus: NARS |
| none | |

SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: GNARSS
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 76.7 |
| COIL: | Lupas's algorithm to detect coiled-coil regions |
| | total: 0 residues |

Final Results (k = 9/23):

44.4%: endoplasmic reticulum
11.1%: Golgi
11.1%: vacuolar
11.1%: mitochondrial
11.1%: nuclear
11.1%: cytoplasmic

>> prediction for CG57111-01 is end (k = 9)

A search of the NOV45a protein against the Geneseq database, a proprietary database that contains sequences published in patents and patent publication, yielded several homologous proteins shown in Table 45D.

TABLE 45D
Geneseq Results for NOV45a

| | | NOV45a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABP70119 | Human NOV32a - Homo sapiens, 947 | 1 . . . 947 | 947/947 (100%) | 0.0 |
| | aa. [WO200272771-A2, 19-SEP-2002] | 1 . . . 947 | 947/947 (100%) | |
| ABU52605 | Human NOVX protein, NOV16a - | 1 . . . 947 | 946/947 (99%) | 0.0 |
| | Homo sapiens, 962 aa. | 16 . . . 962 | 946/947 (99%) | |
| | [WO200281518-A2, 17-OCT-2002] | | | |
| AAU09958 | Human cadherin-1 (CDHN-1) protein - | 24 . . . 947 | 923/924 (99%) | 0.0 |
| | Homo sapiens, 924 aa. | 1 . . . 924 | 923/924 (99%) | |
| | [WO200179293-A2, 25-OCT-2001] | | | |
| ABP47865 | Human polypeptide SEQ ID NO 295 - | 551 . . . 947 | 396/397 (99%) | 0.0 |
| | Homo sapiens, 397 aa. | 1 . . . 397 | 397/397 (99%) | |
| | [US2002042386-A1, 11-APR-2002] | | | |
| AAU19645 | Human novel extracellular matrix | 551 . . . 947 | 396/397 (99%) | 0.0 |
| | protein, Seq ID No 295 - Homo | 1 . . . 397 | 397/397 (99%) | |
| | sapiens, 397 aa. [WO200155368-A1, | | | |
| | 02-AUG-2001] | | | |

In a BLAST search of public sequence databases, the NOV45a protein was found to have homology to the proteins shown in the BLASTP data in Table 45E.

TABLE 45E
Public BLASTP Results for NOV45a

| | | NOV45a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9NRT9 | Protocadherin 13 - Homo sapiens | 1 . . . 947 | 947/947 (100%) | 0.0 |
| | (Human), 947 aa (fragment). | 1 . . . 947 | 947/947 (100%) | |
| Q8NDN4 | Hypothetical protein - Homo | 1 . . . 947 | 946/947 (99%) | 0.0 |
| | sapiens (Human), 951 aa. | 5 . . . 951 | 946/947 (99%) | |
| Q8N6Y1 | Protocadherin 20 - Homo sapiens | 24 . . . 947 | 923/924 (99%) | 0.0 |
| | (Human), 924 aa. | 1 . . . 924 | 923/924 (99%) | |
| Q8BIV2 | PROTOCADHERIN 13 homolog - | 1 . . . 947 | 874/948 (92%) | 0.0 |
| | Mus musculus (Mouse), 952 aa. | 5 . . . 952 | 910/948 (95%) | |
| Q8BIZ0 | PROTOCADHERIN 13 homolog - | 24 . . . 947 | 860/925 (92%) | 0.0 |
| | Mus musculus (Mouse), 925 aa. | 1 . . . 925 | 896/925 (95%) | |

PFam analysis predicts that the NOV45a protein contains the domains shown in the Table 45F.

TABLE 45F
Domain Analysis of NOV45a

| | | Identities/ | |
|---|---|---|---|
| | NOV45a | Similarities for | Expect |
| Pfam Domain | Match Region | the Matched Region | Value |
| cadherin | 210 . . . 307 | 36/111 (32%) | 2.5e−08 |
| | | 72/111 (65%) | |
| cadherin | 321 . . . 413 | 27/110 (25%) | 0.0004 |
| | | 66/110 (60%) | |
| cadherin | 536 . . . 626 | 33/107 (31%) | 6.4e−19 |
| | | 69/107 (64%) | |
| cadherin | 640 . . . 729 | 39/107 (36%) | 5.8e−20 |
| | | 69/107 (64%) | |
| cadherin | 747 . . . 840 | 38/112 (34%) | 4e−11 |
| | | 70/112 (62%) | |

### Example 46

The NOV46 clone was analyzed, and the nucleotide and encoded polypeptide sequences are shown in Table 46A.

TABLE 46A
NOV46 Sequence Analysis

| | | |
|---|---|---|
| NOV46a, CG57409-05 | SEQ ID NO: 1019 | 1500 bp |
| DNA Sequence | ORF Start: ATG at 12 | ORE Stop: TGA at 1476 |

TGAGCCGAGACATGAGCGGGACCTACCGCTGCCAGACGGCCCGCTATAATGGCTTCAACGTGCGCCCC
CGTGAGGCCCAGGTGCAGCTGAACGTGCAGTTCCCGCCGGAGGTGGAGCCCAGTTCCCAGGACGTGCG
CCAGGCGCTGGGCCGGCCCGTGCTCCTGCGCTGCTCGCTGCTGCGAGGCAGCCCCCAGCGCATCGCCT
CGGCTGTGTGGCGTTTCAAAGGGCAGCTGCTGCCGCCGCCGCCTGTTGTTCCCGCCGCCGCCGAGGCG
CCGGATCACGCGGAGCTGCGCCTCGACGCCGTAACTCGCGACAGCAGCGGCAGCTACGAGTGCAGCGT
CTCCAACGATGTGGGCTCGGCTGCCTGCCTCTTCCAGGTCTCCGCCAAAGCCTACAGCCCGGAGTTTT
ACTTCGACACCCCCAACCCCACCCGCAGCCACAAGCTGTCCAAGAACTACTCCTACGTGCTGCAGTGG
ACTCAGAGGGAGCCCGACGCTGTCGACCCTGTGCTCAACTACAGACTCAGCATCCGCCAGTTGAACCA
GCACAATGCGGTGGTCAAGGCCATCCCGGTCCGGCGTGTGGAGAAGGGGCAGCTGCTGGAGTACATCC
TGACCGATCTCCGTGTGCCCCACAGCTATGAGGTCCGCCTCACACCCTATACCACCTTCGGGGCTGGT
GACATGGCCTCCCGCATCATCCACTACACAGAGCGCCAGATCCGCTGGCCCCCAGTCCTGGCTCTGAG
GACCCTGTCCTCTGGTCCCAAGCAGGGTATCCTCTGCAGAGCCCCACACCTCAGTTCTGACTTGGTTT
CCCCGCTTGCTTTCTCAGCCATCAACTCTCCGAACCTTTCAGACAACACCTGCCACTTTGAGGATGAG
AAGATCTGTGGCTATACCCAGGACCTGACAGACAACTTTGACTGGACGCGGCAGAATGCCCTCACCCA
GAACCCCAAACGCTCCCCCAACACTGGTCCCCCCACCGACATAAGTGGCACCCCTGAGGGCTACTACA
TGTTCATCGAGACATCGAGGCCTCGGGAGCTGGGGGACCGTGCAAGGTTAGTGAGTCCCCTCTACAAT
GCCAGCGCCAAGTTCTACTGTGTCTCCTTCTTCTACCACATGTACGGGAAACACATCGGCTCCCTCAA
CCCCCTGGTGCGGTCCCGGAACAAAGGGGCTCTGGACACGCACGCCTGGTCTCTCAGTGGCAATAAGG
GCAATGTGTGGCAGCAGGCCCATGTGCCCATCAGCCCCAGTGGGCCCTTCCAGATTATTTTTGAGGGG
GTTCGAGGCCCGGGCTACCTGGGGGATATTGCCATAGATGACGTCACACTGAAGAAGGGGGAGTGTCC
CCGGAAGCAGACGGATCCCAATAAAGTGGTGGTGATGCCGGGCAGTGGAGCCCCCTGCCAGTCCAGCC
CACAGCTGTGGGGGCCCATGGCCATCTTCCTCTTGGCGTTGCAGAGATGATGAGAGCTGTGTGGCCAC
CCCC

| | | | |
|---|---|---|---|
| NOV46a, CG57409-05 | SEQ ID NO: 1020 | 488 aa | MW at 54357.1kD |
| Protein Sequence | | | |

MSGTYRCQTARYNGFNVRPREAQVQLNVQFPPEVEPSSQDVRQALGRPVLLRCSLLRGSPQRIASAVW
RFKGQLLPPPPVVPAAAEAPDHAELRLDAVTRDSSGSYECSVSNDVGSAACLFQVSAKAYSPEFYFDT
PNPTRSHKLSKNYSYVLQWTQREPDAVDPVLNYRLSIRQLNQHNAVVKAIPVRRVEKGQLLEYILTDL
RVPHSYEVRLTPYTTFGAGDMASRIIHYTERQIRWPPVLALRTLSSGPKQGILCRAPHLSSDLVSPLA
FSAINSPNLSDNTCHFEDEKICGYTQDLTDNFDWTRQNALTQNPKRSPNTGPPTDISGTPEGYYMFIE
TSRPRELGDRARLVSPLYNASAKFYCVSFFYHMYGKHIGSLNPLVRSRNKGALDTHAWSLSGNKGNVW
QQAHVPISPSGPFQIIFEGVRGPGYLGDIAIDDVTLKKGECPRKQTDPNKVVVMPGSGAPCQSSPQLW
GPMAIFLLALQR

| | | |
|---|---|---|
| NOV46b, 277731446 | SEQ ID NO: 1021 | 1363 bp |
| DNA Sequence | ORF Start: at 2 | ORF Stop: end of sequence |

CACCGGTACCATGAGCGGGACCTACCGCTGCCAGACGGCCCGCTATAATGGCTTCAACGTGCGCCCCC
GTGAGGCCCAGGTGCAGCTGAACGTGCAGTTCCCGCCGGAGGTGGAGCCCAGTTCCCAGGACGTGCGC
CAGGCGCTGGGCCGGCCCGTGCTCCTGCGCTGCTCGCTGCTGCGAGGCAGCCCCCAGCGCATCGCCTC
GGCTGTGTGGCGTTTCAAAGGGCAGCTGCTGCCGCCGCCGCCTGTTGTTCCCGCCGCCGCCGAGGCGC
CGGATCACGCGGAGCTGCGCCTCGACGCCGTAACTCGCGACAGCAGCGGCAGCTACGAGTGCAGCGTC
TCCAACGATGTGGGCTCGGCTGCCTGCCTCTTCCAGGTCTCCGCCAAAGCCTACAGCCCGGAGTTTTA
CTTCGACACCCCCAACCCCACCCGCAGCCACAAGCTGTCCAAGAACTACTCCTACGTGCTGCAGTGGA
CTCAGAGGGAGCCCGACGCTGTCGACCCTGTGCTCAACTACAGACTCAGCATCCGCCAGTTGAACCAG
CACAATGCGGTGGTCAAGGCCATCCCGGTCCGGCGTGTGGAGAAGGGGCAGCTGCTGGAGTACATCCT
GACCGATCTCCGTGTGCCCCACAGCTATGAGGTCCGCCTCACACCCTATACCACCTTCGGGGCTGGTG
ACATGGCCTCCCGCATCATCCACTACACAGAGCCCATCAACTCTCCGAACCTTTCAGACAACACCTGC
CACTTTGAGGATGAGAAGATCTGTGGCTATACCCAGGACCTGACAGACAACTTTGACTGGACGCGGCA
GAATGCCCTCACCCAGAACCCCAAACGCTCCCCCAACACTGGTCCCCCCACCGACATAAGTGGCACCC
CTGAGGGCTACTACATGTTCATCGAGACATCGAGGCCTCGGGAGCTGGGGGACCGTGCAAGGTTAGTG
AGTCCCCTCTACAATGCCAGCGCCAAGTTCTACTGTGTCTCCTTCTTCTACCACATGTACGGGAAACA
CATCGGCTCCCTCAACCTCCTGGTGCGGTCCCGGAACAAAGGGGCTCTGGACACGCACGCCTGGTCTC
TCAGTGGCAATAAGGGCAATGTGTGGCAGCAGGCCCATGTGCCCATCAGCCCCAGTGGGCCCTTCCAG
ATTATTTTTGAGGGGGTTCGAGGCCCGGGCTACCTGGGGGATATTGCCATAGATGACGTCACACTGAA
GAAGGGGGAGTGTCCCCGGAAGCAGACGGATCCCAATAAAGTGGTGGTGATGCCGGGCAGTGGAGCCC
CCTGCCAGTCCAGCCCACAGCTGTGGGGGCCCATGGCCATCTTCCTCTTGGCGTTGCAGAGACTCGAG
GGC

| | | | |
|---|---|---|---|
| NOV46b, 277731446 | SEQ ID NO: 1022 | 454 aa | MW at 50606.7kD |
| Protein Sequence | | | |

TGTMSGTYRCQTARYNGFNVRPREAQVQLNVQFPPEVEPSSQDVRQALGRPVLLRCSLLRGSPQRIAS
AVWRFKGQLLPPPPVVPAAAEAPDHAELRLDAVTRDSSGSYECSVSNDVGSAACLFQVSAKAYSPEFY
FDTPNPTRSHKLSKNYSYVLQWTQREPDAVDPVLNYRLSIRQLNQHNAVVKAIPVRRVEKGQLLEYIL
TDLRVPHSYEVRLTPYTTFGAGDMASRIIHYTEPINSPNLSDNTCHFEDEKICGYTQDLTDNFDWTRQ
NALTQNPKRSPNTGPPTDISGTPEGYYMFIETSRPRELGDRARLVSPLYNASAKFYCVSFFYHMYGKH
IGSLNLLVRSRNKGALDTHAWSLSGNKGNVWQQAHVPISPSGPFQIIFEGVRGPGYLGDIAIDDVTLK
KGECPRKQTDPNKVVVMPGSGAPCQSSPQLWGPMAIFLLALQRLEG

| | | |
|---|---|---|
| NOV46c, CG57409-07 | SEQ ID NO: 1023 | 1363 bp |
| DNA Sequence | ORF Start: ATG at 11 | ORF Stop: at 1355 |

CACCGGTACCATGAGCGGGACCTACCGCTGCCAGACGGCCCGCTATAATGGCTTCAACGTGCGCCCCC
GTGAGGCCCAGGTGCAGCTGAACGTGCAGTTCCCGCCGGAGGTGGAGCCCAGTTCCCAGGACGTGCGC
CAGGCGCTGGGCCGGCCCGTGCTCCTGCGCTGCTCGCTGCTGCGAGGCAGCCCCCAGCGCATCGCCTC
GGCTGTGTGGCGTTTCAAAGGGCAGCTGCTGCCGCCGCCGCCTGTTGTTCCCGCCGCCGCCGAGGCGC
CGGATCACGCGGAGCTGCGCCTCGACGCCGTAACTCGCGACAGCAGCGGCAGCTACGAGTGCAGCGTC
TCCAACGATGTGGGCTCGGCTGCCTGCCTCTTCCAGGTCTCCGCCAAAGCCTACAGCCCGGAGTTTTA
CTTCGACACCCCCAACCCCACCCGCAGCCACAAGCTGTCCAAGAACTACTCCTACGTGCTGCAGTGGA
CTCAGAGGGAGCCCGACGCTGTCGACCCTGTGCTCAACTACAGACTCAGCATCCGCCAGTTGAACCAG
CACAATGCGGTGGTCAAGGCCATCCCGGTCCGGCGTGTGGAGAAGGGGCAGCTGCTGGAGTACATCCT
GACCGATCTCCGTGTGCCCCACAGCTATGAGGTCCGCCTCACACCCTATACCACCTTCGGGGCTGGTG
ACATGGCCTCCCGCATCATCCACTACACAGAGCCCATCAACTCTCCGAACCTTTCAGACAACACCTGC
CACTTTGAGGATGAGAAGATCTGTGGCTATACCCAGGACCTGACAGACAACTTTGACTGGACGCGGCA
GAATGCCCTCACCCAGAACCCCAAACGCTCCCCCAACACTGGTCCCCCCACCGACATAAGTGGCACCC
CTGAGGGCTACTACATGTTCATCGAGACATCGAGGCCTCGGGAGCTGGGGGACCGTGCAAGGTTAGTG
AGTCCCCTCTACAATGCCAGCGCCAAGTTCTACTGTGTCTCCTTCTTCTACCACATGTACGGGAAACA
CATCGGCTCCCTCAACCTCCTGGTGCGGTCCCGGAACAAAGGGGCTCTGGACACGCACGCCTGGTCTC
TCAGTGGCAATAAGGGCAATGTGTGGCAGCAGGCCCATGTGCCCATCAGCCCCAGTGGGCCCTTCCAG
ATTATTTTTGAGGGGGTTCGAGGCCCGGGCTACCTGGGGGATATTGCCATAGATGACGTCACACTGAA
GAAGGGGGAGTGTCCCCGGAAGCAGACGGATCCCAATAAAGTGGTGGTGATGCCGGGCAGTGGAGCCC
CCTGCCAGTCCAGCCCACAGCTGTGGGGGCCCATGGCCATCTTCCTCTTGGCGTTGCAGAGACTCGAG
GGC

| | | | |
|---|---|---|---|
| NOV46c, CG57409-07 | SEQ ID NO: 1024 | 448 aa | MW at 50048.1kD |
| Protein Sequence | | | |

MSGTYRCQTARYNGFNVRPREAQVQLNVQFPPEVEPSSQDVRQALGRPVLLRCSLLRGSPQRIASAVW
RFKGQLLPPPPVVPAAAEAPDHAELRLDAVTRDSSGSYECSVSNDVGSAACLFQVSAKAYSPEFYFDT
PNPTRSHKLSKNYSYVLQWTQREPDAVDPVLNYRLSIRQLNQHNAVVKAIPVRRVEKGQLLEYILTDL
RVPHSYEVRLTPYTTFGAGDMASRIIHYTEPINSPNLSDNTCHFEDEKICGYTQDLTDNFDWTRQNAL
TQNPKRSPNTGPPTDISGTPEGYYMFIETSRPRELGDRARLVSPLYNASAKFYCVSFFYHMYGKHIGS
LNLLVRSRNKGALDTHAWSLSGNKGNVWQQAHVPISPSGPFQIIFEGVRGPGYLGDIAIDDVTLKKGE
CPRKQTDPNKVVVMPGSGAPCQSSPQLWGPMAIFLLALQR

| | | |
|---|---|---|
| NOV46d, 312102874 | SEQ ID NO: 1025 | 1369 bp |
| DNA Sequence | ORF Start: at 2 | ORF Stop: end of sequence |

CACCAAGCTTCCCACCATGAGCGGGACCTACCGCTGCCAGACGGCCCGCTATAATGGCTTCAACGTGC
GCCCCCGTGAGGCCCAGGTGCAGCTGAACGTGCAGTTCCCGCCGGAGGTGGAGCCCAGTTCCCAGGAC
GTGCGCCAGGCGCTGGGCCGGCCCGTGCTCCTGCGCTGCTCGCTGCTGCGAGGCAGCCCCCAGCGCAT
CGCCTCGGCTGTGTGGCGTTTCAAAGGGCAGCTGCTGCCGCCGCCGCCTGTTGTTCCCGCCGCCGCCG
AGGCGCCGGATCACGCGGAGCTGCGCCTCGACGCCGTAACTCGCGACAGCAGCGGCAGCTACGAGTGC
AGCGTCTCCAACGATGTGGGCTCGGCTGCCTGCCTCTTCCAGGTCTCCGCCAAAGCCTACAGCCCGGA
GTTTTACTTCGACACCCCCAACCCCACCCGCAGCCACAAGCTGTCCAAGAACTACTCCTACGTGCTGC
AGTGGACTCAGAGGGAGCCCGACGCTGTCGACCCTGTGCTCAACTACAGACTCAGCATCCGCCAGTTG
AACCAGCACAATGCGGTGGTCAAGGCCATCCCGGTCCGGCGTGTGGAGAAGGGGCAGCTGCTGGAGTA
CATCCTGACCGATCTCCGTGTGCCCCACAGCTATGAGGTCCGCCTCACACCCTATACCACCTTCGGGG
CTGGTGACATGGCCTCCCGCATCATCCACTACACAGAGCCCATCAACTCTCCGAACCTTTCAGACAAC
ACCTGCCACTTTGAGGATGAGAAGATCTGTGGCTATACCCAGGACCTGACAGACAACTTTGACTGGAC
GCGGCAGAATGCCCTCACCCAGAACCCCAAACGCTCCCCCAACACTGGTCCCCCCACCGACATAAGTG
GCACCCCTGAGGGCTACTACATGTTCATCGAGACATCGAGGCCTCGGGAGCTGGGGGACCGTGCAAGG
TTAGTGAGTCCCCTCTACAATGCCAGCGCCAAGTTCTACTGTGTCTCCTTCTTCTACCACATGTACGG
GAAACACATCGGCTCCCTCAACCTCCTGGTGCGGTCCCGGAACAAAGGGGCTCTGGACACGCACGCCT
GGTCTCTCAGTGGCAATAAGGGCAATGTGTGGCAGCAGGCCCATGTGCCCATCAGCCCCAGTGGGCCC
TTCCAGATTATTTTTGAGGGGGTTCGAGGCCCGGGCTACCTGGGGGATATTGCCATAGATGACGTCAC
ACTGAAGAAGGGGGAGTGTCCCCGGAAGCAGACGGATCCCAATAAAGTGGTGGTGATGCCGGGCAGTG
GAGCCCCCTGCCAGTCCAGCCCACAGCTGTGGGGGCCCATGGCCATCTTCCTCTTGGCGTTGCAGAGA
GAATTCGGC

| | | | |
|---|---|---|---|
| NOV46d, 312102874 | SEQ ID NO: 1026 | 456 aa | MW at 50922.1kD |
| Protein Sequence | | | |

TKLPTMSGTYRCQTARYNGFNVRPREAQVQLNVQFPPEVEPSSQDVRQALGRPVLLRCSLLRGSPQRI
ASAVWRFKGQLLPPPPVVPAAAEAPDHAELRLDAVTRDSSGSYECSVSNDVGSAACLFQVSAKAYSPE
FYFDTPNPTRSHKLSKNYSYVLQWTQREPDAVDPVLNYRLSIRQLNQHNAVVKATPVRRVEKGQLLEY
ILTDLRVPHSYEVRLTPYTTFGAGDMASRIIHYTEPINSPNLSDNTCHFEDEKICGYTQDLTDNFDWT
RQNALTQNPKRSPNTGPPTDISGTPEGYYMFIETSRPRELGDRARLVSPLYNASAKFYCVSFFYHMYG
KHIGSLNLLVRSRNKGALDTHAWSLSGNKGNVWQQAHVPISPSGPFQIIFEGVRGPGYLGDIAIDDVT
LKKGECPRKQTDPNKVVVMPGSGAPCQSSPQLWGPMAIFLLALQREFG

| | | |
|---|---|---|
| NOV46e, 312102899 | SEQ ID NO: 1027 | 1249 bp |
| DNA Sequence | ORF Start: at 2 | ORF Stop: end of sequence |

CACCAAGCTTAGCGGGACCTACCGCTGCCAGACGGCCCGCTATAATGGCTTCAACGTGCGCCCCCGTG
AGGCCCAGGTGCAGCTGAACGTGCAGTTCCCGCCGGAGGTGGAGCCCAGTTCCCAGGACGTGCGCCAG
GCGCTGGGCCGGCCCGTGCTCCTGCGCTGCTCGCTGCTGCGAGGCAGCCCCCAGCGCATCGCCTCGGC
TGTGTGGCGTTTCAAAGGGCAGCTGCTGCCGCCGCCGCCTGTTGTTCCCGCCGCCGCCGAGGCGCCGG
ATCACGCGGAGCTGCGCCTCGACGCCGTAACTCGCGACAGCAGCGGCAGCTACGAGTGCAGCGTCTCC
AACGATGTGGGCTCGGCTGCCTGCCTCTTCCAGGTCTCCGCCAAAGCCTACAGCCCGGAGTTTTACTT
CGACACCCCCAACCCCACCCGCAGCCACAAGCTGTCCAAGAACTACTCCTACGTGCTGCAGTGGACTC
AGAGGGAGCCCGACGCTGTCGACCCTGTGCTCAACTACAGACTCAGCATCCGCCAGTTGAACCAGCAC
AATGCGGTGGTCAAGGCCATCCCGGTCCGGCGTGTGGAGAAGGGGCAGCTGCTGGAGTACATCCTGAC
CGATCTCCGTGTGCCCCACAGCTATGAGGTCCGCCTCACACCCTATACCACCTTCGGGGCTGGTGACA
TGGCCTCCCGCATCATCCACTACACAGAGCCCATCAACTCTCCGAACCTTTCAGACAACACCTGCCAC
TTTGAGGATGAGAAGATCTGTGGCTATACCCAGGACCTGACAGACAACTTTGACTGGACGCGGCAGAA
TGCCCTCACCCAGAACCCCAAACGCTCCCCCAACACTGGTCCCCCCACCGACATAAGTGGCACCCCTG
AGGGCTACTACATGTTCATCGAGACATCGAGGCCTCGGGAGCTGGGGGACCGTGCAAGGTTAGTGAGT
CCCCTCTACAATGCCAGCGCCAAGTTCTACTGTGTCTCCTTCTTCTACCACATGTACGGGAAACACAT
CGGCTCCCTCAACCTCCTGGTGCGGTCCCGGAACAAAGGGGCTCTGGACACGCACGCCTGGTCTCTCA
GTGGCAATAAGGGCAATGTGTGGCAGCAGGCCCATGTGCCCATCAGCCCCAGTGGGCCCTTCCAGATT
ATTTTTGAGGGGGTTCGAGGCCCGGGCTACCTGGGGGATATTGCCATAGATGACGTCACACTGAAGAA
GGGGGAGTGTCCCCGGGAATTCGGC

| | | | |
|---|---|---|---|
| NOV46e, 312102899 | SEQ ID NO: 1028 | 416 aa | MW at 46644.0kD |
| Protein Sequence | | | |

TKLSGTYRCQTARYNGFNVRPREAQVQLNVQFPPEVEPSSQDVRQALGRPVLLRCSLLRGSPQRIASA
VWRFKGQLLPPPPVVPAAAEAPDHAELRLDAVTRDSSGSYECSVSNDVGSAACLFQVSAKAYSPEFYF
DTPNPTRSHKLSKNYSYVLQWTQREPDAVDPVLNYRLSIRQLNQHNAVVKAIPVRRVEKGQLLEYILT
DLRVPHSYEVRLTPYTTFGAGDMASRIIHYTEPINSPNLSDNTCHFEDEKICGYTQDLTDNFDWTRQN
ALTQNPKRSPNTGPPTDISGTPEGYYMFIETSRPRELGDRARLVSPLYNASAKFYCVSFFYHMYGKHI
GSLNLLVRSRNKGALDTHAWSLSGNKGNVWQQAHVPISPSGPFQIIFEGVRGPGYLGDIAIDDVTLKK
GECPREFG

| | | |
|---|---|---|
| NOV46f, CG57409-01 | SEQ ID NO: 1029 | 4141 bp |
| DNA Sequence | ORF Start: ATG at 135 | ORF Stop: at 4140 |

TCTTCGTCGCCGCTCTCTCTCTCACCTCTCAGGGAAAGGGGGGGACATAGGGGCGTCGCGGGGCCCCC
GCGAATGCGCCCCCCGCCGCCTCTCGGGCTGCGCCGCCTCGCGGGGATGAAGCACCGGCCGTGAAGAT
GGAGGTGACCTGCCTTCTACTTCTGGCGCTGATCCCCTTCCACTGCCGGGGACAAGGAGTCTACGCTC
CAGCCCAGGCGCAGATCGTGCATGCGGGCCAGGCATGTGTGGTGAAAGAGGACAATATCAGCGAGCGT
GTCTACACCATCCGGGAGGGGGACACCCTCATGCTGCAGTGCCTTGTAACAGGGCACCCTCGACCCCA
GGTACGGTGGACCAAGACGGCAGGTAGCGCCTCGGACAAGTTCCAGGAGACATCGGTGTTCAACGAGA
CGCTGCGCATCGAGCGTATTGCACGCACGCAGGGCGGCCGCTACTACTGCAAGGCTGAGAACGGCGTG
GGGGTGCCGGCCATCAAGTCCATCCGCGTGGACGTGCAGTACCTGGATGAGCCAATGCTGACGGTGCA
CCAGACGGTGAGCGATGTGCGAGGCAACTTCTACCAGGAGAAGACGGTGTTCCTGCGCTGTACTGTCA
ACTCCAACCCGCCTGCCCGCTTCATCTGGAAGCGGGGTTCCGATACCCTATCCCACAGCCAGGACAAT
GGGGTTGACATCTATGAGCCCCTCTACACTCAGGGGGAGACCAAGGTCCTGAAGCTGAAGAACCTGCG
GCCCCAGGACTATGCCAGCTACACCTGCCAGGTGTCTGTGCGTAACGTGTGCGGCATCCCAGACAAGG
CCATCACCTTCCGGCTCACCAACACCACGGCACCACCAGCCCTGAAGCTGTCTGTGAACGAAACTCTG
GTGGTGAACCCTGGGGAGAATGTGACGGTGCAGTGTCTGCTGACAGGCGGTGATCCCCTCCCCCAGCT
GCAGTGGTCCCATGGGCCTGGCCCACTGCCCCTGGGTGCTCTGGCCCAGGGTGGCACCCTCAGCATCC
CTTCAGTGCAGGCCCGGGACTCTGGCTACTACAACTGCACAGCCACCAACAATGTGGGCAACCCTGCC
AAGAAGACTGTCAACCTGCTGGTGCGATCCATGAAGAACGCTACATTCCAGATCACTCCTGACGTGAT
CAAAGAGAGTGAGAACATCCAGCTGGGCCAGGACCTGAAGCTATCGTGCCACGTGGATGCAGTGCCCC
AGGAGAAGGTGACCTACCAGTGGTTCAAGAATGGCAAGCCGGCACGCATGTCCAAGCGGCTGCTGGTG
ACCCGCAATGATCCTGAGCTGCCCGCAGTCACCAGCAGCCTAGAGCTCATTGACCTGCACTTCAGTGA
CTATGGCACCTACCTGTGCATGGCTTCTTTCCCAGGGGCACCCGTGCCCGACCTCAGCGTCGAGGTCA
ACATCTCCTCTGAGACAGTGCCGCCCACCATCAGTGTGCCCAAGGGTAGGGCCGTGGTGACCGTGCGC
GAGGGATCGCCTGCCGAGCTGCAATGCGAGGTGCGGGGCAAGCCGCGGCCGCCAGTGCTCTGGTCCCG
CGTGGACAAGGAGGCTGCACTGCTGCCCTCGGGGCTGCCCCTGGAGGAGACTCCGGACGGGAAGCTGC
GGCTGGAGCGAGTGAGCCGAGACATGAGCGGGACCTACCGCTGCCAGACGGCCCGCTATAATGGCTTC
AACGTGCGCCCCCGTGAGGCCCAGGTGCAGCTGAACGTGCAGTTCCCGCCGGAGGTGGAGCCCAGTTC
CCAGGACGTGCGCCAGGCGCTGGGCCGGCCCGTGCTCCTGCGCTGCTCGCTGCTGCGAGGCAGCCCCC
AGCGCATCGCCTCGGCTGTGTGGCGTTTCAAAGGGCAGCTGCTGCCGCCGCCGCCTGTTGTTCCCGCC
GCCGCCGAGGCGCCGGATCACGCGGAGCTGCGCCTCGACGCCGTAACTCGCGACAGCAGCGGCAGCTA
CGAGTGCAGCGTCTCCAACGATGTGGGCTCGGCTGCCTGCCTCTTCCAGGTCTCCGCCAAAGCCTACA
GCCCGGAGTTTTACTTCGACACCCCCAACCCCACCCGCAGCCACAAGCTGTCCAAGAACTACTCCTAC
GTGCTGCAGTGGACTCAGAGGGAGCCCGACGCTGTCGACCCTGTGCTCAACTACAGACTCAGCATCCG
CCAGTTGAACCAGCACAATGCGGTGGTCAAGGCCATCCCGGTCCGGCGTGTGGAGAAGGGGCAGCTGC
TGGAGTACATCCTGACCGATCTCCGTGTGCCCCACAGCTATGAGGTCCGCCTCACACCCTATACCACC
TTCGGGGCTGGTGACATGGCCTCCCGCATCATCCACTACACAGAGCGCCAGATCCGCTGGCCCCCAGT
CCTGGCTCTGAGGACCCTGTCCTCTGGTCCCAAGCAGGGTATCCTCTGCAGAGCCCCACACCTCAGTT
CTGACTTGGTTTCCCCGCTTGCTTTCTCAGCCATCAACTCTCCGAACCTTTCAGACAACACCTGCCAC
TTTGAGGATGAGAAGATCTGTGGCTATACCCAGGACCTGACAGACAACTTTGACTGGACGCGGCAGAA
TGCCCTCACCCAGAACCCCAAACGCTCCCCCAACACTGGTCCCCCCACCGACATAAGTGGCACCCCTG
AGGGCTACTACATGTTCATCGAGACATCGAGGCCTCGGGAGCTGGGGGACCGTGCAAGGTTAGTGAGT
CCCCTCTACAATGCCAGCGCCAAGTTCTACTGTGTCTCCTTCTTCTACCACATGTACGGGAAACACAT
CGGCTCCCTCAACCTCCTGGTGCGGTCCCGGAACAAAGGGGCTCTGGACACGCACGCCTGGTCTCTCA
GTGGCAATAAGGGCAATGTGTGGCAGCAGGCCCATGTGCCCATCAGCCCCAGTGGGCCCTTCCAGATT
ATTTTTGAGGGGGTTCGAGGCCCGGGCTACCTGGGGGATATTGCCATAGATGACGTCACACTGAAGAA
GGGGGAGTGTCCCCGGAAGCAGACGGATCCCAATAAAGGTGCAAGACGGGAAGGAGCTGCCTGCGATG
GCCTGAAATTCCACCTTTCATCCCCTATGGATGACGGAGAGCTTACAGATGACCCTATTGAATGCAAG
CACCTTTGGATCCATAGAGTGGACAGTAAAGGTGCTCAGTACATGTTGGCTGAGCTGAACTGCATACA
TGTGGCCCCCAGGTTCCTGGTCTTTATGGACGAAGGGCACAAGGTTGGTGAAAAGGACTCCGGGGGCC
AGCCCTTCCAAGTTTACACTGATTTCTCCTTTTACCCTCATGCTATCCCTGAGAAGATGTCAATAATG
CCCACGTTACAGGTGGGAAAACTGAGGCTTAGAGAGGAGGAGGAATCTGCCTACGGTCACACAGCTGC
AAAGGCTAGAGCTGGGACCAGGAGCTGGTCTCTTAACCGACCACCTGAGCTCAAGAGCTTTTCTCTCT
GGACCAACATGACCCAAAGTGTGCGCGAGCCTATCACAGGTCCCCTGCAATGCCAAACATACACGCAC
AGCAATACACAACACCTGGGGACATGGATGAAGCTGGAAACCATCATTCTCAGCAAACTGACACAAGA
ACAGAAAACCAAACACCACATGTTCTCACTCACCACCCAGTCTGCCCCGCCCTCTCTCTTCTCACCTG
AACTTCCCCTCTCCTCAAACTCTCGAGGCCACGCCTCTATGTCCTTGGATGATGATGATGACGACGAC
GACGATGATGATGATGATGATGACGACGATGACAATGATGATGATGATGGAAGGAAGACCTACAGAAT
CCCTCCAGGCTCTGACCTCAGTGCTTGTGGGTGGGTGAATGACCACATGTCGCAGGGAGACTCCACAG
GTCCTCCCGATGAGAAGCACTCTTATGCCAAAGAGGAGACTCAGGCCAAACTGACAGGACCAGGAATT
AGCTACCCTGGTAAACCCAGCTATCGACTGCACCCGAGCGGCTACACACCACTGGAGCAGTTCAGGGA
GAAAGCCACCGGCATGCTCACCCCGTATGTCTCTGGCTCTGTTTCCTCTTTCTGCTTCCCCTTCCCCA
CCTCTGAGTCTCTGTGTTCTGCTCATGCCAATTCCCCTTCTGCCTGTCTCTGCCCGCTTCT

| | | | |
|---|---|---|---|
| NOV46f, CG57409-01 | SEQ ID NO: 1030 | 1335 aa | MW at 147969.4kD |
| Protein Sequence | | | |

MEVTCLLLLALIPFHCRGQGVYAPAQAQIVHAGQACVVKEDNISERVYTIREGDTLMLQCLVTGHPRP
QVRWTKTAGSASDKFQETSVFNETLRIERIARTQGGRYYCKAENGVGVPAIKSIRVDVQYLDEPMLTV
HQTVSDVRGNFYQEKTVFLRCTVNSNPPARFIWKRGSDTLSHSQDNGVDIYEPLYTQGETKVLKLKNL
RPQDYASYTCQVSVRNVCGIPDKAITFRLTNTTAPPALKLSVNETLVVNPGENVTVQCLLTGGDPLPQ
LQWSHGPGPLPLGALAQGGTLSIPSVQARDSGYYNCTATNNVGNPAKKTVNLLVRSMKNATFQITPDV
IKESENIQLGQDLKLSCHVDAVPQEKVTYQWFKNGKPARMSKRLLVTRNDPELPAVTSSLELIDLHFS
DYGTYLCMASFPGAPVPDLSVEVNISSETVPPTISVPKGRAVVTVREGSPAELQCEVRGKPRPPVLWS
RVDKEAALLPSGLPLEETPDGKLRLERVSRDMSGTYRCQTARYNGFNVRPREAQVQLNVQFPPEVEPS
SQDVRQALGRPVLLRCSLLRGSPQRIASAVWRFKGQLLPPPPVVPAAAEAPDHAELRLDAVTRDSSGS
YECSVSNDVGSAACLFQVSAKAYSPEFYFDTPNPTRSHKLSKNYSYVLQWTQREPDAVDPVLNYRLSI
RQLNQHNAVVKAIPVRRVEKGQLLEYILTDLRVPHSYEVRLTPYTTFGAGDMASRIIHYTERQIRWPP
VLALRTLSSGPKQGILCRAPHLSSDLVSPLAFSAINSPNLSDNTCHFEDEKICGYTQDLTDNFDWTRQ
NALTQNPKRSPNTGPPTDISGTPEGYYMFIETSRPRELGDRARLVSPLYNASAKFYCVSFFYHMYGKH
IGSLNLLVRSRNKGALDTHAWSLSGNKGNVWQQAHVPISPSGPFQIIFEGVRGPGYLGDIAIDDVTLK
KGECPRKQTDPNKGARREGAACDGLKFHLSSPMDDGELTDDPIECKHLWIHRVDSKGAQYMLAELNCI
HVAPRFLVFMDEGHKVGEKDSGGQPFQVYTDFSFYPHAIPEKMSIMPTLQVGKLRLREEEESAYGHTA
AKARAGTRSWSLNRPPELKSFSLWTNMTQSVREPITGPLQCQTYTHSNTQHLGTWNKLETIILSKLTQ
EQKTKHHMFSLTTQSAPPSLFSPELPLSSNSRGHASMSLDDDDDDDDDDDDDDDDDDNDDDDGRKTYR
IPPGSDLSACGWVNDHMSQGDSTGPPDEKHSYAKEETQAKLTGPGISYPGKPSYRLHPSGYTPLEQFR
EKATGMLTPYVSGSVSSFCFPFPTSESLCSAHANSPSACLCPL

| | | |
|---|---|---|
| NOV46g, CG57409-02 | SEQ ID NO: 1031 | 720 bp |
| DNA Sequence | ORF Start: at 7 | ORF Stop: at 715 |

GGTACCTTGAACCAGCACAATGCGGTGGTCAAGGCCATCCCGGTCCGGCGTGTGGAGAAGGGGCAGCT
GCTGGAGTACATCCTGACCGATCTCCGTGTGCCCCACAGCTATGAGGTCCGCCTCACACCCTATACCA
CCTTCGGGGCTGGTGACATGGCCTCCCGCATCATCCACTACACAGAGCCCATCAACTCTCCGAACCTT
TCAGACAACACCTGCCACTTTGAGGATGAGAAGATCTGTGGCTATACCCAGGACCTGACAGACAACTT
TGACTGGACGCGGCAGAATGCCCTCACCCAGAACCCCAAACGCTCCCCCAACACTGGTCCCCCCACCG
ACATAAGTGGCACCCCTGAGGGCTACTACATGTTCATCGAGACATCGAGGCCTCGGGAGCTGGGGGAC
CGTGCAAGGTTAGTGAGTCCCCTCTACAATGCCTGCGCCAAGTTCTACTGTGTCTCCTTCTTCTACCA
CATGTACGGGAAACACATCGGCTCCCTCAACCTCCTGGTGCGGTCCCGGAACAAAGGGGCTCTGGACA
CGCACGCCTGGTCTCTCAGTGGCAATAAGGGCAATGTGTGGCAGCAGGCCCATGTGCCCATCAGCCCC
AGTGGGCCCTTCCAGATTATTTTTGAGGGGGTTCGAGGCCCGGGCTACCTGGGGGATATTGCCATAGA
TGACGTCACACTGAAGAAGGGGGAGTGTCCCCGGGAATTC

| | | | |
|---|---|---|---|
| NOV46g, CG57409-02 | SEQ ID NO: 1032 | 236 aa | MW at 26563.7kD |
| Protein Sequence | | | |

LNQHNAVVKAIPVRRVEKGQLLEYILTDLRVPHSYEVRLTPYTTFGAGDMASRIIHYTEPINSPNLSD
NTCHFEDEKICGYTQDLTDNFDWTRQNALTQNPKRSPNTGPPTDISGTPEGYYMFIETSRPRELGDRA
RLVSPLYNACAKFYCVSFFYHMYGKHIGSLNLLVRSRNKGALDTHAWSLSGNKGNVWQQAHVPISPSG
PFQIIFEGVRGPGYLGDIAIDDVTLKKGECPR

| | | |
|---|---|---|
| NOV46h, CG57409-03 | SEQ ID NO: 1033 | 4169 bp |
| DNA Sequence | ORF Start: ATG at 135 | ORF Stop: TGA at 4080 |

TCTTCGTCGCCGCTCTCTCTCTCACCTCTCAGGGAAAGGGGGGGACATAGGGGCGTCGCGGGGCCCCG
GCGAATGCGCCCCCCGCCGCCTCTCGGGCTGCGCCGCCTCGCGGGGATGAAGCACCGGCCGTGAAGAT
GGAGGTGACCTGCCTTCTACTTCTGGCGCTGATCCCCTTCCACTGCCGGGGACAAGGAGTCTACGCTC
CAGCCCAGGCGCAGATCGTGCATGCGGGCCAGGCATGTGTGGTGAAAGAGGACAATATCAGCGAGCGT
GTCTACACCATCCGGGAGGGGGACACCCTCATGCTGCAGTGCCTTGTAACAGGGCACCCTCGACCCCA
GGTACGGTGGACCAAGACGGCAGGTAGCGCCTCGGACAAGTTCCAGGAGACATCGGTGTTCAACGAGA
CGCTGCGCATCGAGCGTATTGCACGCACGCAGGGCGGCCGCTACTACTGCAAGGCTGAGAACGGCGTG
GGGGTGCCGGCCATCAAGTCCATCCGCGTGGACGTGCAGTACCTGGATGAGCCAATGCTGACGGTGCA
CCAGACGGTGAGCGATGTGCGAGGCAACTTCTACCAGGAGAAGACGGTGTTCCTGCGCTGTACTGTCA
ACTCCAACCCGCCTGCCCGCTTCATCTGGAAGCGGGGTTCCGATACCCTATCCCACAGCCAGGACAAT
GGGGTTGACATCTATGAGCCCCTCTACACTCAGGGGGAGACCAAGGTCCTGAAGCTGAAGAACCTGCG
GCCCCAGGACTATGCCAGCTACACCTGCCAGGTGTCTGTGCGTAACGTGTGCGGCATCCCAGACAAGG
CCATCACCTTCCGGCTCACCAACACCACGGCACCACCAGCCCTGAAGCTGTCTGTGAACGAAACTCTG
GTGGTGAACCCTGGGGAGAATGTGACGGTGCAGTGTCTGCTGACAGGCGGTGATCCCCTCCCCCAGCT
GCAGTGGTCCCATGGGCCTGGCCCACTGCCCCTGGGTGCTCTGGCCCAGGGTGGCACCCTCAGCATCC
CTTCAGTGCAGGCCCGGGACTCTGGCTACTACAACTGCACAGCCACCAACAATGTGGGCAACCCTGCC
AAGAAGACTGTCAACCTGCTGGTGCGATCCATGAAGAACGCTACATTCCAGATCACTCCTGACGTGAT
CAAAGAGAGTGAGAACATCCAGCTGGGCCAGGACCTGAAGCTATCGTGCCACGTGGATGCAGTGCCCC
AGGAGAAGGTGACCTACCAGTGGTTCAAGAATGGCAAGCCGGCACGCATGTCCAAGCGGCTGCTGGTG
ACCCGCAATGATCCTGAGCTGCCCGCAGTCACCAGCAGCCTAGAGCTCATTGACCTGCACTTCAGTGA
CTATGGCACCTACCTGTGCATGGCTTCTTTCCCAGGGGCACCCGTGCCCGACCTCAGCGTCGAGGTCA
ACATCTCCTCTGAGACAGTGCCGCCCACCATCAGTGTGCCCAAGGGTAGGGCCGTGGTGACCGTGCGC
GAGGGATCGCCTGCCGAGCTGCAATGCGAGGTGCGGGGCAAGCCGCGGCCGCCAGTGCTCTGGTCCCG
CGTGGACAAGGAGGCTGCACTGCTGCCCTCGGGGCTGCCCCTGGAGGAGACTCCGGACGGGAAGCTGC
GGCTGGAGCGAGTGAGCCGAGACATGAGCGGGACCTACCGCTGCCAGACGGCCCGCTATAATGGCTTC
AACGTGCGCCCCCGTGAGGCCCAGGTGCAGCTGAACGTGCAGTTCCCGCCGGAGGTGGAGCCCAGTTC
CCAGGACGTGCGCCAGGCGCTGGGCCGGCCCGTGCTCCTGCGCTGCTCGCTGCTGCGAGGCAGCCCCC
AGCGCATCGCCTCGGCTGTGTGGCGTTTCAAAGGGCAGCTGCTGCCGCCGCCGCCTGTTGTTCCCGCC
GCCGCCGAGGCGCCGGATCACGCGGAGCTGCGCCTCGACGCCGTAACTCGCGACAGCAGCGGCAGCTA
CGAGTGCAGCGTCTCCAACGATGTGGGCTCGGCTGCCTGCCTCTTCCAGGTCTCCGCCAAAGCCTACA
GCCCGGAGTTTTACTTCGACACCCCCAACCCCACCCGCAGCCACAAGCTGTCCAAGAACTACTCCTAC
GTGCTGCAGTGGACTCAGAGGGAGCCCGACGCTGTCGACCCTGTGCTCAACTACAGACTCAGCATCCG
CCAGTTGAACCAGCACAATGCGGTGGTCAAGGCCATCCCGGTCCGGCGTGTGGAGAAGGGGCAGCTGC
TGGAGTACATCCTGACCGATCTCCGTGTGCCCCACAGCTATGAGGTCCGCCTCACACCCTATACCACC
TTCGGGGCTGGTGACATGGCCTCCCGCATCATCCACTACACAGAGCCCATCAACTCTCCGAACCTTTC
AGACAACACCTGCCACTTTGAGGATGAGAAGATCTGTGGCTATACCCAGGACCTGACAGACAACTTTG
ACTGGACGCGGCAGAATGCCCTCACCCAGAACCCCAAACGCTCCCCCAACACTGGTCCCCCCACCGAC
ATAAGTGGCACCCCTGAGGGCTACTACATGTTCATCGAGACATCGAGGCCTCGGGAGCTGGGGGACCG
TGCAAGGTTAGTGAGTCCCCTCTACAATGCCAGCGCCAAGTTCTACTGTGTCTCCTTCTTCTACCACA
TGTACGGGAAACACATCGGCTCCCTCAACCTCCTGGTGCGGTCCCGGAACAAAGGGGCTCTGGACACG
CACGCCTGGTCTCTCAGTGGCAATAAGGGCAATGTGTGGCAGCAGGCCCATGTGCCCATCAGCCCCAG
TGGGCCCTTCCAGATTATTTTTGAGGGGGTTCGAGGCCCGGGCTACCTGGGGGATATTGCCATAGATG
ACGTCACACTGAAGAAGGGGGAGTGTCCCCGGAAGCAGACGGATCCCAATAAAGGTGCAAGACGGGAA
GGAGCTGCCTGCGATGGCCTGAAATTCCACCTTTCATCCCCTATGGATGACGGAGAGCTTACAGATGA
CCCTATTGAATGCAAGCACCTTTGGATCCATAGAGTGGACAGTAAAGGTGCTCAGTACATGTTGGCTG
AGCTGAACTGCATACATGTGGCCCCCAGGTTCCTGGTCTTTATGGACGAAGGGCACAAGGTTGGTGAA
AAGGACTCCGGGGGCCAGCCCTTCCAAGTTTACACTGATTTCTCCTTTTACCCTCATGCTATCCCTGA
GAAGATGTCAATAATGCCCACGTTACAGGTGGGAAAACTGAGGCTTAGAGAGGAGGAGGAATCTGCCT
ACGGTCACACAGCTGCAAAGGCTAGAGCTGGGACCAGGAGCTGGTCTCTTAACCGACCACCTGAGCTC
AAGAGCTTTTCTCTCTGGACCAACATGACCCAAAGTGTGCGCGAGCCTATCACAGGTCCCCTGCAATG
CCAAACATACACGCACAGCAATACACAACACCTGGGGACATGGATGAAGCTGGAAACCATCATTCTCA
GCAAACTGACACAAGAACAGAAAACCAAACACCACATGTTCTCACTCACCACCCAGTCTGCCCCGCCC
TCTCTCTTCTCACCTGAACTTCCCCTCTCCTCAAACTCTCGAGGCCACGCCTCTATGTCCTTGGATGA
TGATGATGACGACGACGACGATGATGATGATGATGATGACGACGATGACAATGATGATGATGATGGAA
GGAAGACCTACAGAATCCCTCCAGGCTCTGACCTCAGTGCTTGTGGGTGGGTGAATGACCACATGTCG
CAGGGAGACTCCACAGGTCCTCCCGATGAGAAGCACTCTTATGCCAAAGAGGAGACTCAGGCCAAACT
GACAGGACCAGGAATTAGCTACCCTGGTAAACCCAGCTATCGACTGCACCCGAGCGGCTACACACCAC
TGGAGCAGTTCAGGGAGAAAGCCACCGGCATGCTCACCCCGTATGTCTCTGGCTCTGTTTCCTCTTTC
TGCTTCCCCTTCCCCACCTCTGAGTCTCTGTGTTCTGCTCATGCCAATTCCCCTTCTGCCTGTCTCTG
CCCGCTTCTCTCTGGGCTGGTCTCTCCGAGACTCTGTTCCCTTGGCTGGCATGCCCTCCACCTCCCCT
GATGGTTCAGCAGAGATGAAGCCGGCCTGGCTCATGGGTGTGGGTAATGTACTAGTGCAGGAGAGTGG
TGGGGCCCAGTCTGGGTGCAG

| | | | |
|---|---|---|---|
| NOV46h, CG57409-03 | SEQ ID NO: 1034 | 1315 aa | MW at 145782.9kD |
| Protein Sequence | | | |

MEVTCLLLLALIPFHCRGQGVYAPAQAQIVHAGQACVVKEDNISERVYTIREGDTLMLQCLVTGHPRP
QVRWTKTAGSASDKFQETSVFNETLRIERIARTQGGRYYCKAENGVGVPAIKSIRVDVQYLDEPMLTV
HQTVSDVRGNFYQEKTVFLRCTVNSNPPARFIWKRGSDTLSHSQDNGVDIYEPLYTQGETKVLKLKNL
RPQDYASYTCQVSVRNVCGIPDKAITFRLTNTTAPPALKLSVNETLVVNPGENVTVQCLLTGGDPLPQ
LQWSHGPGPLPLGALAQGGTLSIPSVQARDSGYYNCTATNNVGNPAKKTVNLLVRSMKNATFQITPDV
IKESENIQLGQDLKLSCHVDAVPQEKVTYQWFKNGKPARMSKRLLVTRNDPELPAVTSSLELIDLHFS
DYGTYLCMASFPGAPVPDLSVEVNISSETVPPTISVPKGRAVVTVREGSPAELQCEVRGKPRPPVLWS
RVDKEAALLPSGLPLEETPDGKLRLERVSRDMSGTYRCQTARYNGFNVRPREAQVQLNVQFPPEVEPS
SQDVRQALGRPVLLRCSLLRGSPQRIASAVWRFKGQLLPPPPVVPAAAEAPDHAELRLDAVTRDSSGS
YECSVSNDVGSAACLFQVSAKAYSPEFYFDTPNPTRSHKLSKNYSYVLQWTQREPDAVDPVLNYRLSI
RQLNQHNAVVKAIPVRRVEKGQLLEYILTDLRVPHSYEVRLTPYTTFGAGDMASRIIHYTEPINSPNL
SDNTCHFEDEKICGYTQDLTDNFDWTRQNALTQNPKRSPNTGPPTDISGTPEGYYMFIETSRPRELGD
RARLVSPLYNASAKFYCVSFFYHMYGKHIGSLNLLVRSRNKGALDTHAWSLSGNKGNVWQQAHVPISP
SGPFQIIFEGVRGPGYLGDIAIDDVTLKKGECPRKQTDPNKGARREGAACDGLKFHLSSPMDDGELTD
DPIECKHLWIHRVDSKGAQYMLAELNCIHVAPRFLVFMDEGHKVGEKDSGGQPFQVYTDFSFYPHAIP
EKMSIMPTLQVGKLRLREEEESAYGHTAAKARAGTRSWSLNRPPELKSFSLWTNMTQSVREPITGPLQ
CQTYTHSNTQHLGTWMKLETIILSKLTQEQKTKHHNFSLTTQSAPPSLFSPELPLSSNSRGHASMSLD
DDDDDDDDDDDDDDDDDNDDDDGRKTYRIPPGSDLSACGWVNDHMSQGDSTGPPDEKHSYAKEETQAK
LTGPGISYPGKPSYRLHPSGYTPLEQFREKATGMLTPYVSGSVSSFCFPFPTSESLCSAHANSPSACL
CPLLSGLVSPRLCSLGWHALHLP

| | | |
|---|---|---|
| NOV46i, CG57409-04 | SEQ ID NO: 1035 | 720 bp |
| DNA Sequence | ORF Start: at 7 | ORF Stop: at 715 |

GGATCCTTGAACCAGCACAATGCGGTGGTCAAGGCCATCCCGGTCCGGCGTGTGGAGAAGGGGCAGCT
GCTGGAGTACATCCTGACCGATCTCCGTGTGCCCCACAGCTATGAGGTCCGCCTCACACCCTATACCA
CCTTCGGGGCTGGTGACATGGCCTCCCGCATCATCCACTACACAGAGCCCATCAACTCTCCGAACCTT
TCAGACAACACCTGCCACTTTGAGGATGAGAAGATCTGTGGCTATACCCAGGACCTGACAGACAACTT
TGACTGGACGCGGCAGAATGCCCTCACCCAGAACCCCAAACGCTCCCCCAACACTGGTCCCCCCACCG
ACATAAGTGGCACCCCTGAGGGCTACTACATGTTCATCGAGACATCGAGGCCTCGGGAGCTGGGGGAC
CGTGCAAGGTTAGTGAGTCCCCTCTACAATGCCTGCGCCAAGTTCTACTGTGTCTCCTTCTTCTACCA
CATGTACGGGAAACACATCGGCTCCCTCAACCTCCTGGTGCGGTCCCGGAACAAAGGGGCTCTGGACA
CGCACGCCTGGTCTCTCAGTGGCAATAAGGGCAATGTGTGGCAGCAGGCCCATGTGCCCATCAGCCCC
AGTGGGCCCTTCCAGATTATTTTTGAGGGGGTTCGAGGCCCGGGCTACCTGGGGGATATTGCCATAGA
TGACGTCACACTGAAGAAGGGGGAGTGTCCCCGGCTCGAG

| | | | |
|---|---|---|---|
| NOV46i, CG57409-04 | SEQ ID NO: 1036 | 236 aa | MW at 26563.7kD |
| Protein Sequence | | | |

LNQHNAVVKAIPVRRVEKGQLLEYILTDLRVPHSYEVRLTPYTTFGAGDMASRIIHYTEPINSPNLSD
NTCHFEDEKICGYTQDLTDNFDWTRQNALTQNPKRSPNTGPPTDISGTPEGYYMFIETSRPRELGDRA
RLVSPLYNACAKFYCVSFFYHMYGKHIGSLNLLVRSRNKGALDTHAWSLSGNKGNVWQQAHVPISPSG
PFQIIFEGVRGPGYLGDIAIDDVTLKKGECPR

| | | |
|---|---|---|
| NOV46j, CG57409-06 | SEQ ID NO: 1037 | 1828 bp |
| DNA Sequence | ORF Start: ATG at 12 | ORF Stop: TGA at 1521 |

TGAGCCGAGACATGAGCGGGACCTACCGCTGCCAGACGGCCCGCTATAATGGCTTCAACGTGCGCCCC
CGTGAGGCCCAGGTGCAGCTGAACGTGCAGTTCCCGCCGGAGGTGGAGCCCAGTTCCCAGGACGTGCG
CCAGGCGCTGGGCCGGCCCGTGCTCCTGCGCTGCTCGCTGCTGCGAGGCAGCCCCCAGCGCATCGCCT
CGGCTGTGTGGCGTTTCAAAGGGCAGCTGCTGCCGCCGCCGCCTGTTGTTCCCGCCGCCGCCGAGGCG
CCGGATCACGCGGAGCTGCGCCTCGACGCCGTAACTCGCGACAGCAGCGGCAGCTACGAGTGCAGCGT
CTCCAACGATGTGGGCTCGGCTGCCTGCCTCTTCCAGGTCTCCGCCAAAGCCTACAGCCCGGAGTTTT
ACTTCGACACCCCCAACCCCACCCGCAGCCACAAGCTGTCCAAGAACTACTCCTACGTGCTGCAGTGG
ACTCAGAGGGAGCCCGACGCTGTCGACCCTGTGCTCAACTACAGACTCAGCATCCGCCAGTTGAACCA
GCACAATGCGGTGGTCAAGGCCATCCCGGTCCGGCGTGTGGAGAAGGGGCAGCTGCTGGAGTACATCC
TGACCGATCTCCGTGTGCCCCACAGCTATGAGGTCCGCCTCACACCCTATACCACCTTCGGGGCTGGT
GACATGGCCTCCCGCATCATCCACTACACAGAGCGCCAGATCCGCTGGCCCCCAGTCCTGGCTCTGAG
GACCCTGTCCTCTGGTCCCAAGCAGGGTATCCTCTGCAGAGCCCCACACCTCAGTTCTGACTTGGTTT
CCCCGCTTGCTTTCTCAGCCATCAACTCTCCGAACCTTTCAGACAACACCTGCCACTTTGAGGATGAG
AAGATCTGTGGCTATACCCAGGACCTGACAGACAACTTTGACTGGACGCGGCAGAATGCCCTCACCCA
GAACCCCAAACGCTCCCCCAACACTGGTCCCCCCACCGACATAAGTGGCACCCCTGAGGGCTACTACA
TGTTCATCGAGACATCGAGGCCTCGGGAGCTGGGGGACCGTGCAAGGTTAGTGAGTCCCCTCTACAAT
GCCAGCGCCAAGTTCTACTGTGTCTCCTTCTTCTACCACATGTACGGGAAACACATCGGCTCCCTCAA
CCCCCTGGTGCGGTCCCGGAACAAAGGGGCTCTGGACACGCACGCCTGGTCTCTCAGTGGCAATAAGG
GCAATGTGTGGCAGCAGGCCCATGTGCCCATCAGCCCCAGTGGGCCCTTCCAGATTATTTTTGAGGGG
GTTCGAGGCCCGGGCTACCTGGGGGATATTGCCATAGATGACGTCACACTGAAGAAGGGGGAGTGTCC
CCGGAAGCAGACGGATCCCAATAAAGGTGCAAGACGGGAAGGAGGTGGGGGAGCTGAATCTGGAGGGA
GCTGTGCGTGGCGGGGGTTCCTGTCTGTTGAGGGAGGGTGTTCGGGTCTGAATAGGGGTTCAGACTGT
CTGATGATGGGAATCAGGTGGCTCTGACTGTGTTAACGTGTGCCCACAACTCACGTCAGGCTGAGAAC
TGGTGTAACACCATGAGAAAGCTTGGCCCCCACCATCGTGATGAGCATACCGACCTGGTCACCGGAAC
ACAAACACCAACAACCACAGAGGGCGCCTCAGAATACCCAGAGGGCCCAATACGCCGACCCGCTGTCA
CGAGCGCCCACGAGCGGCAGAACACGACAGGCACACAACCAGCCGGAGCAAGACGGAGCCGAGAGCCC
CGGGGACATAGACCCCAGCAAGCGACACACAAGGACGCGCACAGAGCGCACACACTAACA

| | | | |
|---|---|---|---|
| NOV46j, CG57409-06 | SEQ ID NO: 1038 | 503 aa | MW at 55764.4kD |
| Protein Sequence | | | |

MSGTYRCQTARYNGFNVRPREAQVQLNVQFPPEVEPSSQDVRQALGRPVLLRCSLLRGSPQRIASAVW
RFKGQLLPPPPVVPAAAEAPDHAELRLDAVTRDSSGSYECSVSNDVGSAACLFQVSAKAYSPEFYFDT
PNPTRSHKLSKNYSYVLQWTQREPDAVDPVLNYRLSIRQLNQHNAVVKAIPVRRVEKGQLLEYILTDL
RVPHSYEVRLTPYTTFGAGDMASRIIHYTERQIRWPPVLALRTLSSGPKQGILCRAPHLSSDLVSPLA
FSAINSPNLSDNTCHFEDEKICGYTQDLTDNFDWTRQNALTQNPKRSPNTGPPTDISGTPEGYYMFIE
TSRPRELGDRARLVSPLYNASAKFYCVSFFYHMYGKHIGSLNPLVRSRNKGALDTHAWSLSGNKGNVW
QQAHVPISPSGPFQIIFEGVRGPGYLGDIAIDDVTLKKGECPRKQTDPNKGARREGGGGAESGGSCAW
RGFLSVEGGCSGLNRGSDCLNMGIRWL

| | | |
|---|---|---|
| NOV46k, CG57409-08 | SEQ ID NO: 1039 | 1369 bp |
| DNA Sequence | ORF Start: ATG at 17 | ORF Stop: end of sequence |

CACCAAGCTTCCCACCATGAGCGGGACCTACCGCTGCCAGACGGCCCGCTATAATGGCTTCAACGTGC
GCCCCCGTGAGGCCCAGGTGCAGCTGAACGTGCAGTTCCCGCCGGAGGTGGAGCCCAGTTCCCAGGAC
GTGCGCCAGGCGCTGGGCCGGCCCGTGCTCCTGCGCTGCTCGCTGCTGCGAGGCAGCCCCCAGCGCAT
CGCCTCGGCTGTGTGGCGTTTCAAAGGGCAGCTGCTGCCGCCGCCGCCTGTTGTTCCCGCCGCCGCCG
AGGCGCCGGATCACGCGGAGCTGCGCCTCGACGCCGTAACTCGCGACAGCAGCGGCAGCTACGAGTGC
AGCGTCTCCAACGATGTGGGCTCGGCTGCCTGCCTCTTCCAGGTCTCCGCCAAAGCCTACAGCCCGGA
GTTTTACTTCGACACCCCCAACCCCACCCGCAGCCACAAGCTGTCCAAGAACTACTCCTACGTGCTGC
AGTGGACTCAGAGGGAGCCCGACGCTGTCGACCCTGTGCTCAACTACAGACTCAGCATCCGCCAGTTG
AACCAGCACAATGCGGTGGTCAAGGCCATCCCGGTCCGGCGTGTGGAGAAGGGGCAGCTGCTGGAGTA
CATCCTGACCGATCTCCGTGTGCCCCACAGCTATGAGGTCCGCCTCACACCCTATACCACCTTCGGGG
CTGGTGACATGGCCTCCCGCATCATCCACTACACAGAGCCCATCAACTCTCCGAACCTTTCAGACAAC
ACCTGCCACTTTGAGGATGAGAAGATCTGTGGCTATACCCAGGACCTGACAGACAACTTTGACTGGAC
GCGGCAGAATGCCCTCACCCAGAACCCCAAACGCTCCCCCAACACTGGTCCCCCCACCGACATAAGTG
GCACCCCTGAGGGCTACTACATGTTCATCGAGACATCGAGGCCTCGGGAGCTGGGGGACCGTGCAAGG
TTAGTGAGTCCCCTCTACAATGCCAGCGCCAAGTTCTACTGTGTCTCCTTCTTCTACCACATGTACGG
GAAACACATCGGCTCCCTCAACCTCCTGGTGCGGTCCCGGAACAAAGGGGCTCTGGACACGCACGCCT
GGTCTCTCAGTGGCAATAAGGGCAATGTGTGGCAGCAGGCCCATGTGCCCATCAGCCCCAGTGGGCCC
TTCCAGATTATTTTTGAGGGGGTTCGAGGCCCGGGCTACCTGGGGGATATTGCCATAGATGACGTCAC
ACTGAAGAAGGGGGAGTGTCCCCGGAAGCAGACGGATCCCAATAAAGTGGTGGTGATGCCGGGCAGTG
GAGCCCCCTGCCAGTCCAGCCCACAGCTGTGGGGGCCCATGGCCATCTTCCTCTTGGCGTTGCAGAGA
GAATTCGGC

| | | | |
|---|---|---|---|
| NOV46k, CG57409-08 | SEQ ID NO: 1040 | 451 aa | MW at 50381.4kD |
| Protein Sequence | | | |

MSGTYRCQTARYNGFNVRPREAQVQLNVQFPPEVEPSSQDVRQALGRPVLLRCSLLRGSPQRIASAVW
RFKGQLLPPPPVVPAAAEAPDHAELRLDAVTRDSSGSYECSVSNDVGSAACLFQVSAKAYSPEFYFDT
PNPTRSHKLSKNYSYVLQWTQREPDAVDPVLNYRLSIRQLNQHNAVVKAIPVRRVEKGQLLEYILTDL
RVPHSYEVRLTPYTTFGAGDMASRIIHYTEPINSPNLSDNTCHFEDEKICGYTQDLTDNFDWTRQNAL
TQNPKRSPNTGPPTDISGTPEGYYMFIETSRPRELGDRARLVSPLYNASAKFYCVSFFYHMYGKHIGS
LNLLVRSRNKGALDTHAWSLSGNKGNVWQQAHVPISPSGPFQIIFEGVRGPGYLGDIAIDDVTLKKGE
CPRKQTDPNKVVVMPGSGAPCQSSPQLWGPMAIFLLALQREFG

A ClustalW comparison of the above protein sequences yields the following sequence alignment shown in Table 46B.

TABLE 46B
Comparison of the NOV46 protein sequences.

| | |
|---|---|
| NOV46a | ------------------------------------------------------------ |
| NOV46b | ------------------------------------------------------------ |
| NOV46c | ------------------------------------------------------------ |
| NOV46d | ------------------------------------------------------------ |
| NOV46e | ------------------------------------------------------------ |
| NOV46f | MEVTCLLLLALIPFHCRGQGVYAPAQAQIVHAGQACVVKEDNISERVYTIREGDTLMLQC |
| NOV46g | ------------------------------------------------------------ |
| NOV46h | MEVTCLLLLALIPFHCRGQGVYAPAQAQIVHAGQACVVKEDNISERVYTIREGDTLMLQC |
| NOV46i | ------------------------------------------------------------ |
| NOV46j | ------------------------------------------------------------ |
| NOV46k | ------------------------------------------------------------ |
| NOV46a | ------------------------------------------------------------ |
| NOV46b | ------------------------------------------------------------ |
| NOV46c | ------------------------------------------------------------ |
| NOV46d | ------------------------------------------------------------ |
| NOV46e | ------------------------------------------------------------ |
| NOV46f | LVTGHPRPQVRWTKTAGSASDKFQETSVFNETLRIERIARTQGGRYYCKAENGVGVPAIK |
| NOV46g | ------------------------------------------------------------ |
| NOV46h | LVTGHPRPQVRWTKTAGSASDKFQETSVFNETLRIERIARTQGGRYYCKAENGVGVPAIK |
| NOV46i | ------------------------------------------------------------ |
| NOV46j | ------------------------------------------------------------ |
| NOV46k | ------------------------------------------------------------ |
| NOV46a | ------------------------------------------------------------ |
| NOV46b | ------------------------------------------------------------ |
| NOV46c | ------------------------------------------------------------ |
| NOV46d | ------------------------------------------------------------ |
| NOV46e | ------------------------------------------------------------ |
| NOV46f | SIRVDVQYLDEPMLTVHQTVSDVRGNFYQEKTVFLRCTVNSNPPARFIWKRGSDTLSHSQ |
| NOV46g | ------------------------------------------------------------ |
| NOV46h | SIRVDVQYLDEPMLTVHQTVSDVRGNFYQEKTVFLRCTVNSNPPARFIWKRGSDTLSHSQ |
| NOV46i | ------------------------------------------------------------ |
| NOV46j | ------------------------------------------------------------ |
| NOV46k | ------------------------------------------------------------ |
| NOV46a | ------------------------------------------------------------ |
| NOV46b | ------------------------------------------------------------ |
| NOV46c | ------------------------------------------------------------ |
| NOV46d | ------------------------------------------------------------ |
| NOV46e | ------------------------------------------------------------ |
| NOV46f | DNGVDIYEPLYTQGETKVLKLKNLRPQDYASYTCQVSVRNVCGIPDKAITFRLTNTTAPP |
| NOV46g | ------------------------------------------------------------ |
| NOV46h | DNGVDIYEPLYTQGETKVLKLKNLRPQDYASYTCQVSVRNVCGIPDKAITFRLTNTTAPP |
| NOV46i | ------------------------------------------------------------ |
| NOV46j | ------------------------------------------------------------ |
| NOV46k | ------------------------------------------------------------ |
| NOV46a | ------------------------------------------------------------ |
| NOV46b | ------------------------------------------------------------ |
| NOV46c | ------------------------------------------------------------ |
| NOV46d | ------------------------------------------------------------ |
| NOV46e | ------------------------------------------------------------ |
| NOV46f | ALKLSVNETLVVNPGENVTVQCLLTGGDPLPQLQWSHGPGPLPLGALAQGGTLSIPSVQA |
| NOV46g | ------------------------------------------------------------ |
| NOV46h | ALKLSVNETLVVNPGENVTVQCLLTGGDPLPQLQWSHGPGPLPLGALAQGGTLSIPSVQA |
| NOV46i | ------------------------------------------------------------ |
| NOV46j | ------------------------------------------------------------ |
| NOV46k | ------------------------------------------------------------ |
| NOV46a | ------------------------------------------------------------ |
| NOV46b | ------------------------------------------------------------ |
| NOV46c | ------------------------------------------------------------ |
| NOV46d | ------------------------------------------------------------ |
| NOV46e | ------------------------------------------------------------ |
| NOV46f | RDSGYYNCTATNNVGNPAKKTVNLLVRSMKNATFQITPDVIKESENIQLGQDLKLSCHVD |
| NOV46g | ------------------------------------------------------------ |
| NOV46h | RDSGYYNCTATNNVGNPAKKTVNLLVRSMKNATFQITPDVIKESENIQLGQDLKLSCHVD |
| NOV46i | ------------------------------------------------------------ |
| NOV46j | ------------------------------------------------------------ |
| NOV46k | ------------------------------------------------------------ |
| NOV46a | ------------------------------------------------------------ |
| NOV46b | ------------------------------------------------------------ |
| NOV46c | ------------------------------------------------------------ |
| NOV46d | ------------------------------------------------------------ |
| NOV46e | ------------------------------------------------------------ |
| NOV46f | AVPQEKVTYQWFKNGKPARMSKRLLVTRNDPELPAVTSSLELIDLHFSDYGTYLCMASFP |
| NOV46g | ------------------------------------------------------------ |
| NOV46h | AVPQEKVTYQWFKNGKPARMSKRLLVTRNDPELPAVTSSLELIDLHFSDYGTYLCMASFP |
| NOV46i | ------------------------------------------------------------ |
| NOV46j | ------------------------------------------------------------ |
| NOV46k | ------------------------------------------------------------ |
| NOV46a | ------------------------------------------------------------ |
| NOV46b | ------------------------------------------------------------ |
| NOV46c | ------------------------------------------------------------ |
| NOV46d | ------------------------------------------------------------ |
| NOV46e | ------------------------------------------------------------ |
| NOV46f | GAPVPDLSVEVNISSETVPPTISVPKGRAVVTVREGSPAELQCEVRGKPRPPVLWSRVDK |
| NOV46g | ------------------------------------------------------------ |
| NOV46h | GAPVPDLSVEVNISSETVPPTISVPKGRAVVTVREGSPAELQCEVRGKPRPPVLWSRVDK |
| NOV46i | ------------------------------------------------------------ |
| NOV46j | ------------------------------------------------------------ |
| NOV46k | ------------------------------------------------------------ |
| NOV46a | ---------------------------MSGTYRCQTARYNGFNVRPREAQVQLNVQFPPE |
| NOV46b | ------------------------TGTMSGTYRCQTARYNGFNVRPREAQVQLNVQFPPE |
| NOV46c | ---------------------------MSGTYRCQTARYNGFNVRPREAQVQLNVQFPPE |
| NOV46d | ----------------------TKLPTMSGTYRCQTARYNGFNVRPREAQVQLNVQFPPE |
| NOV46e | -------------------------TKLSGTYRCQTARYNGFNVRPREAQVQLNVQFPPE |
| NOV46f | EAALLPSGLPLEETPDGKLRLERVSRDMSGTYRCQTARYNGFNVRPREAQVQLNVQFPPE |
| NOV46g | ------------------------------------------------------------ |
| NOV46h | EAALLPSGLPLEETPDGKLRLERVSRDMSGTYRCQTARYNGFNVRPREAQVQLNVQFPPE |
| NOV46i | ------------------------------------------------------------ |
| NOV46j | ---------------------------MSGTYRCQTARYNGFNVRPREAQVQLNVQFPPE |
| NOV46k | ---------------------------MSGTYRCQTARYNGFNVRPREAQVQLNVQFPPE |
| NOV46a | VEPSSQDVRQALGRPVLLRCSLLRGSPQRIASAVWRFKGQLLPPPPVVPAAAEAPDHAEL |
| NOV46b | VEPSSQDVRQALGRPVLLRCSLLRGSPQRIASAVWRFKGQLLPPPPVVPAAAEAPDHAEL |
| NOV46c | VEPSSQDVRQALGRPVLLRCSLLRGSPQRIASAVWRFKGQLLPPPPVVPAAAEAPDHAEL |
| NOV46d | VEPSSQDVRQALGRPVLLRCSLLRGSPQRIASAVWRFKGQLLPPPPVVPAAAEAPDHAEL |
| NOV46e | VEPSSQDVRQALGRPVLLRCSLLRGSPQRIASAVWRFKGQLLPPPPVVPAAAEAPDHAEL |
| NOV46f | VEPSSQDVRQALGRPVLLRCSLLRGSPQRIASAVWRFKGQLLPPPPVVPAAAEAPDHAEL |
| NOV46g | ------------------------------------------------------------ |
| NOV46h | VEPSSQDVRQALGRPVLLRCSLLRGSPQRIASAVWRFKGQLLPPPPVVPAAAEAPDHAEL |
| NOV46i | ------------------------------------------------------------ |
| NOV46j | VEPSSQDVRQALGRPVLLRCSLLRGSPQRIASAVWRFKGQLLPPPPVVPAAAEAPDHAEL |
| NOV46k | VEPSSQDVRQALGRPVLLRCSLLRGSPQRIASAVWRFKGQLLPPPPVVPAAAEAPDHAEL |
| NOV46a | RLDAVTRDSSGSYECSVSNDVGSAACLFQVSAKAYSPEFYFDTPNPTRSHKLSKNYSYVL |
| NOV46b | RLDAVTRDSSGSYECSVSNDVGSAACLFQVSAKAYSPEFYFDTPNPTRSHKLSKNYSYVL |
| NOV46c | RLDAVTRDSSGSYECSVSNDVGSAACLFQVSAKAYSPEFYFDTPNPTRSHKLSKNYSYVL |
| NOV46d | RLDAVTRDSSGSYECSVSNDVGSAACLFQVSAKAYSPEFYFDTPNPTRSHKLSKNYSYVL |
| NOV46e | RLDAVTRDSSGSYECSVSNDVGSAACLFQVSAKAYSPEFYFDTPNPTRSHKLSKNYSYVL |
| NOV46f | RLDAVTRDSSGSYECSVSNDVGSAACLFQVSAKAYSPEFYFDTPNPTRSHKLSKNYSYVL |
| NOV46g | ------------------------------------------------------------ |
| NOV46h | RLDAVTRDSSGSYECSVSNDVGSAACLFQVSAKAYSPEFYFDTPNPTRSHKLSKNYSYVL |
| NOV46i | ------------------------------------------------------------ |
| NOV46j | RLDAVTRDSSGSYECSVSNDVGSAACLFQVSAKAYSPEFYFDTPNPTRSHKLSKNYSYVL |
| NOV46k | RLDAVTRDSSGSYECSVSNDVGSAACLFQVSAKAYSPEFYFDTPNPTRSHKLSKNYSYVL |
| NOV46a | QWTQREPDAVDPVLNYRLSIRQLNQHNAVVKAIPVRRVEKGQLLEYILTDLRVPHSYEVR |
| NOV46b | QWTQREPDAVDPVLNYRLSIRQLNQHNAVVKAIPVRRVEKGQLLEYILTDLRVPHSYEVR |
| NOV46c | QWTQREPDAVDPVLNYRLSIRQLNQHNAVVKAIPVRRVEKGQLLEYILTDLRVPHSYEVR |
| NOV46d | QWTQREPDAVDPVLNYRLSIRQLNQHNAVVKAIPVRRVEKGQLLEYILTDLRVPHSYEVR |
| NOV46e | QWTQREPDAVDPVLNYRLSIRQLNQHNAVVKAIPVRRVEKGQLLEYILTDLRVPHSYEVR |
| NOV46f | QWTQREPDAVDPVLNYRLSIRQLNQHNAVVKAIPVRRVEKGQLLEYILTDLRVPHSYEVR |
| NOV46g | ----------------------LNQHNAVVKAIPVRRVEKGQLLEYILTDLRVPHSYEVR |
| NOV46h | QWTQREPDAVDPVLNYRLSIRQLNQHNAVVKAIPVRRVEKGQLLEYILTDLRVPHSYEVR |
| NOV46i | ----------------------LNQHNAVVKAIPVRRVEKGQLLEYILTDLRVPHSYEVR |
| NOV46j | QWTQREPDAVDPVLNYRLSIRQLNQHNAVVKAIPVRRVEKGQLLEYILTDLRVPHSYEVR |
| NOV46k | QWTQREPDAVDPVLNYRLSIRQLNQHNAVVKAIPVRRVEKGQLLEYILTDLRVPHSYEVR |
| NOV46a | LTPYTTFGAGDMASRIIHYTERQIRWPPVLALRTLSSGPKQGILCRAPHLSSDLVSPLAF |
| NOV46b | LTPYTTFGAGDMASRIIHYTEP-------------------------------------- |
| NOV46c | LTPYTTFGAGDMASRIIHYTEP-------------------------------------- |
| NOV46d | LTPYTTFGAGDMASRIIHYTEP-------------------------------------- |
| NOV46e | LTPYTTFGAGDMASRIIHYTEP-------------------------------------- |
| NOV46f | LTPYTTFGAGDMASRIIHYTERQIRWPPVLALRTLSSGPKQGILCRAPHLSSDLVSPLAF |
| NOV46g | LTPYTTFGAGDMASRIIHYTEP---------IN--------------------------- |
| NOV46h | LTPYTTFGAGDMASRIIHYTEP---------IN--------------------------- |
| NOV46i | LTPYTTFGAGDMASRIIHYTEP-------------------------------------- |
| NOV46j | LTPYTTFGAGDMASRIIHYTERQIRWPPVLALRTLSSGPKQGILCRAPHLSSDLVSPLAF |
| NOV46k | LTPYTTFGAGDMASRIIHYTEP-------------------------------------- |
| NOV46a | SAINSPNLSDNTCHFEDEKICGYTQDLTDNFDWTRQNALTQNPKRSPNTGPPTDISGTPE |
| NOV46b | --INSPNLSDNTCHFEDEKICGYTQDLTDNFDWTRQNALTQNPKRSPNTGPPTDISGTPE |
| NOV46c | --INSPNLSDNTCHFEDEKICGYTQDLTDNFDWTRQNALTQNPKRSPNTGPPTDISGTPE |
| NOV46d | --INSPNLSDNTCHFEDEKICGYTQDLTDNFDWTRQNALTQNPKRSPNTGPPTDISGTPE |
| NOV46e | --INSPNLSDNTCHFEDEKICGYTQDLTDNFDWTRQNALTQNPKRSPNTGPPTDISGTPE |
| NOV46f | SAINSPNLSDNTCHFEDEKICGYTQDLTDNFDWTRQNALTQNPKRSPNTGPPTDISGTPE |
| NOV46g | ----SPNLSDNTCHFEDEKICGYTQDLTDNFDWTRQNALTQNPKRSPNTGPPTDISGTPE |
| NOV46h | ----SPNLSDNTCHFEDEKICGYTQDLTDNFDWTRQNALTQNPKRSPNTGPPTDISGTPE |
| NOV46i | --INSPNLSDNTCHFEDEKICGYTQDLTDNFDWTRQNALTQNPKRSPNTGPPTDISGTPE |
| NOV46j | SAINSPNLSDNTCHFEDEKICGYTQDLTDNFDWTRQNALTQNPKRSPNTGPPTDISGTPE |
| NOV46k | --INSPNLSDNTCHFEDEKICGYTQDLTDNFDWTRQNALTQNPKRSPNTGPPTDISGTPE |
| NOV46a | GYYMFIETSRPRELGDRARLVSPLYNASAKFYCVSFFYHMYGKHIGSLNPLVRSRNKGAL |
| NOV46b | GYYMFIETSRPRELGDRARLVSPLYNASAKFYCVSFFYHMYGKHIGSLNLLVRSRNKGAL |
| NOV46c | GYYMFIETSRPRELGDRARLVSPLYNASAKFYCVSFFYHMYGKHIGSLNLLVRSRNKGAL |
| NOV46d | GYYMFIETSRPRELGDRARLVSPLYNASAKFYCVSFFYHNYGKHIGSLNLLVRSRNKGAL |
| NOV46e | GYYMFIETSRPRELGDRARLVSPLYNASAKFYCVSFFYHMYGKHIGSLNLLVRSRNKGAL |
| NOV46f | GYYMFIETSRPRELGDRARLVSPLYNASAKFYCVSFFYHMYGKHIGSLNLLVRSPNKGAL |
| NOV46g | GYYMFIETSRPRELGDRARLVSPLYNACAKFYCVSFFYHMYGKHIGSLNLLVRSRNKGAL |
| NOV46h | GYYMFIETSRPRELGDRARLVSPLYNASAKFYCVSFFYHMYGKHIGSLNLLVRSRNKGAL |
| NOV46i | GYYMFIETSRPRELGDRARLVSPLYNACAKFYCVSFFYHMYGKHIGSLNLLVRSRNKGAL |
| NOV46j | GYYMFIETSRPRELGDRARLVSPLYNASAKFYCVSFFYHNYGKHIGSLNPLVRSRNKGAL |
| NOV46k | GYYMFIETSRPRELGDRARLVSPLYNASAKFYCVSFFYHNYGKHIGSLNLLVRSRNKGAL |
| NOV46a | DTHAWSLSGNKGNVWQQAHVPISPSGPFQIIFEGVRGPGYLGDIAIDDVTLKKGECPRKQ |
| NOV46b | DTHAWSLSGNKGNVWQQAHVPISPSGPFQIIFEGVRGPGYLGDIAIDDVTLKKGECPRKQ |
| NOV46c | DTHAWSLSGNKGNVWQQAHVPISPSGPFQIIFEGVRGPGYLGDIAIDDVTLKKGECPRKQ |
| NOV46d | DTHAWSLSGNKGNVWQQAHVPISPSGPFQIIFEGVRGPGYLGDIAIDDVTLKKGECPRKQ |
| NOV46e | DTHAWSLSGNKGNVWQQAHVPISPSGPFQIIFEGVRGPGYLGDIAIDDVTLKKGECPREF |
| NOV46f | DTHAWSLSGNKGNVWQQAHVPISPSGPFQIIFEGVRGPGYLGDIAIDDVTLKKGECPRKQ |
| NOV46g | DTHAWSLSGNKGNVWQQAHVPISPSGPFQIIFEGVRGPGYLGDIAIDDVTLKKGECPR-- |
| NOV46h | DTHAWSLSGNKGNVWQQAHVPISPSGPFQIIFEGVRGPGYLGDIAIDDVTLKKGECPRKQ |
| NOV46i | DTHAWSLSGNKGNVWQQAHVPISPSGPFQIIFEGVRGPGYLGDIAIDDVTLKKGECPR-- |
| NOV46j | DTHAWSLSGNKGNVWQQAHVPISPSGPFQIIFEGVRGPGYLGDIAIDDVTLKKGECPRKQ |
| NOV46k | DTHAWSLSGNKGNVWQQAHVPISPSGPFQIIFEGVRGPGYLGDIAIDDVTLKKGECPRKQ |
| NOV46a | TDPNKVVVMPGSGAPCQSSPQLWG--------------------PMAIFLLALQR----- |
| NOV46b | TDPNKVVVMPGSGAPCQSSPQLWG--------------------PMAIFLLALQRLEG-- |
| NOV46c | TDPNKVVVMPGSGAPCQSSPQLWG--------------------PMAIFLLALQR----- |
| NOV46d | TDPNKVVVMPGSGAPCQSSPQLWG--------------------PMAIFLLALQREFG-- |
| NOV46e | G----------------------------------------------------------- |
| NOV46f | TDPNKGARREGAACDGLKFHLSSPMDDGELTDDPIECKHLWIHRVDSKGAQYMLAELNCI |
| NOV46g | ------------------------------------------------------------ |
| NOV46h | TDPNKGARREGAACDGLKFHLSSPMDDGELTDDPIECKHLWIHRVDSKGAQYMLAELNCI |
| NOV46i | ------------------------------------------------------------ |
| NOV46j | TDPNKGARREGGGGAESGGSCAWRG-----------------FLSVEGGCSGLNRGSDCL |
| NOV46k | TDPNKVVVMPGSGAPCQSSPQLWG--------------------PMAIFLLALQREFG-- |
| NOV46a | ------------------------------------------------------------ |
| NOV46b | ------------------------------------------------------------ |
| NOV46c | ------------------------------------------------------------ |
| NOV46d | ------------------------------------------------------------ |
| NOV46e | ------------------------------------------------------------ |
| NOV46f | HVAPRFLVFMDEGHKVGEKDSGGQPFQVYTDFSFYPHAIPEKMSIMPTLQVGKLRLREEE |
| NOV46g | ------------------------------------------------------------ |
| NOV46h | HVAPRFLVFMDEGHKVGEKDSGGQPFQVYTDFSFYPHAIPEKMSIMPTLQVGKLRLREEE |
| NOV46i | ------------------------------------------------------------ |
| NOV46j | MMGIRWL----------------------------------------------------- |
| NOV46k | ------------------------------------------------------------ |
| NOV46a | ------------------------------------------------------------ |
| NOV46b | ------------------------------------------------------------ |
| NOV46c | ------------------------------------------------------------ |
| NOV46d | ------------------------------------------------------------ |
| NOV46e | ------------------------------------------------------------ |
| NOV46f | ESAYGHTAAKAPAGTRSWSLNRPPELKSFSLWTNMTQSVREPITGPLQCQTYTHSNTQHL |
| NOV46g | ------------------------------------------------------------ |
| NOV46h | ESAYGHTAAKARAGTRSWSLNRPPELKSFSLWTNMTQSVREPITGPLQCQTYTHSNTQHL |
| NOV46i | ------------------------------------------------------------ |
| NOV46j | ------------------------------------------------------------ |
| NOV46k | ------------------------------------------------------------ |
| NOV46a | ------------------------------------------------------------ |
| NOV46b | ------------------------------------------------------------ |
| NOV46c | ------------------------------------------------------------ |
| NOV46d | ------------------------------------------------------------ |
| NOV46e | ------------------------------------------------------------ |
| NOV46f | GTWMKLETIILSKLTQEQKTKHHMFSLTTQSAPPSLFSPELPLSSNSRGHASMSLDDDDD |
| NOV46g | ------------------------------------------------------------ |
| NOV46h | GTWMKLETIILSKLTQEQKTKHHMFSLTTQSAPPSLFSPELPLSSNSRGHASMSLDDDDD |
| NOV46i | ------------------------------------------------------------ |
| NOV46j | ------------------------------------------------------------ |
| NOV46k | ------------------------------------------------------------ |
| NOV46a | ------------------------------------------------------------ |
| NOV46b | ------------------------------------------------------------ |
| NOV46c | ------------------------------------------------------------ |
| NOV46d | ------------------------------------------------------------ |
| NOV46e | ------------------------------------------------------------ |
| NOV46f | DDDDDDDDDDDDDNDDDDGRKTYRIPPGSDLSACGWVNDHMSQGDSTGPPDEKHSYAKEE |
| NOV46g | ------------------------------------------------------------ |
| NOV46h | DDDDDDDDDDDDDNDDDDGRKTYRIPPGSDLSACGWVNDHMSQGDSTGPPDEKHSYAKEE |
| NOV46i | ------------------------------------------------------------ |
| NOV46j | ------------------------------------------------------------ |
| NOV46k | ------------------------------------------------------------ |
| NOV46a | ------------------------------------------------------------ |
| NOV46b | ------------------------------------------------------------ |
| NOV46c | ------------------------------------------------------------ |
| NOV46d | ------------------------------------------------------------ |
| NOV46e | ------------------------------------------------------------ |
| NOV46f | TQAKLTGPGISYPGKPSYRLHPSGYTPLEQFREKATGMLTPYVSGSVSSFCFPFPTSESL |
| NOV46g | ------------------------------------------------------------ |
| NOV46h | TQAKLTGPGISYPGKPSYRLHPSGYTPLEQFREKATGMLTPYVSGSVSSFCFPFPTSESL |
| NOV46i | ------------------------------------------------------------ |
| NOV46j | ------------------------------------------------------------ |
| NOV46k | ------------------------------------------------------------ |
| NOV46a | ----------------------------------- |
| NOV46b | ----------------------------------- |
| NOV46c | ----------------------------------- |
| NOV46d | ----------------------------------- |
| NOV46e | ----------------------------------- |
| NOV46f | CSAHANSPSACLCPL-------------------- |
| NOV46g | ----------------------------------- |
| NOV46h | CSAHANSPSACLCPLLSGLVSPRLCSLGWHALHLP |
| NOV46i | ----------------------------------- |
| NOV46j | ----------------------------------- |
| NOV46k | ----------------------------------- |
| NOV46a | (SEQ ID NO: 1020) |
| NOV46b | (SEQ ID NO: 1022) |
| NOV46c | (SEQ ID NO: 1024) |
| NOV46d | (SEQ ID NO: 1026) |
| NOV46e | (SEQ ID NO: 1028) |
| NOV46f | (SEQ ID NO: 1030) |
| NOV46g | (SEQ ID NO: 1032) |
| NOV46h | (SEQ ID NO: 1034) |
| NOV46i | (SEQ ID NO: 1036) |
| NOV46j | (SEQ ID NO: 1038) |
| NOV46k | (SEQ ID NO: 1040) |

Further analysis of the NOV46a protein yielded the following properties shown in Table 46C.

TABLE 46C
Protein Sequence Properties NOV46a

| | |
|---|---|
| SignalP analysis: | No Known Signal Sequence Predicted |

PSORT II analysis:

| PSG: | a new signal peptide prediction method | |
|---|---|---|
| | N-region: | length 11; pos. chg 2; neg. chg 0 |
| | H-region: | length 6; peak value −8.10 |
| | PSG score: | −12.50 |

| | |
|---|---|
| GvH: | von Heijne's method for signal seq. recognition |
| | GvH score (threshold: −2.1): −2.49 |
| | possible cleavage site: between 61 and 62 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 0 | |
| | number of TMS(s) . . . fixed | |
| | PERIPHERAL | Likelihood = 6.21 (at 260) |

| | ALOM score: 6.21 (number of TMSs: 0} | | | |
|---|---|---|---|---|
| MITDISC: | discrimination of mitochondrial targeting seq | | | |
| | R content: | 4 | Hyd Moment(75): | 3.44 |
| | Hyd Moment(95): | 5.36 | G content: | 2 |
| | D/E content: | 1 | S/T content: | 3 |

Score: −1.85

Gavel: prediction of cleavage sites for mitochondrial preseq

R-2 motif at 28 VRP|RE

NUCDISC: discrimination of nuclear localization signals

pat4: none
pat7: none
bipartite: none
content of basic residues: 10.9%
NLS Score: −0.47

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear |
| | discrimination |
| | Prediction: nuclear |
| | Reliability: 76.7 |
| COIL: | Lupas's algorithm to detect coiled-coil regions |
| | total: 0 residues |

Final Results (k = 9/23):

47.8%: mitochondrial
47.8%: nuclear
4.3%: cytoplasmic

>> prediction for CG57409-05 is mit (k = 23)

A search of the NOV46a protein against the Geneseq database, a proprietary database that contains sequences published in patents and patent publication, yielded several homologous proteins shown in Table 46D.

TABLE 46D
Geneseq Results for NOV46a

| | | | Identities/ | |
|---|---|---|---|---|
| | | NOV46a | Similarities | |
| Geneseq | Protein/Organism/Length | Residues/ | for the | Expect |
| Identifier | [Patent #, Date] | Match Residues | Matched Region | Value |
| ABU52337 | Human GPCR related protein | 1 . . . 488 | 488/488 (100%) | 0.0 |
| | NOV13b - Homo sapiens, 488 aa. | 1 . . . 488 | 488/488 (100%) | |
| | [WO200279398-A2, 10-OCT-2002] | | | |
| ABU52338 | Human GPCR related protein | 1 . . . 476 | 461/476 (96%) | 0.0 |
| | NOV13c - Homo sapiens, 503 aa. | 1 . . . 476 | 461/476 (96%) | |
| | [WO200279398-A2, 10-OCT-2002] | | | |
| ABJ10922 | Human secreted protein (SECP) #18 - | 1 . . . 458 | 457/458 (99%) | 0.0 |
| | Homo sapiens, 869 aa. | 308 . . . 765 | 457/458 (99%) | |
| | [WO200270669-A2, 12-SEP-2002] | | | |
| ABG12100 | Novel human diagnostic protein | 1 . . . 458 | 457/458 (99%) | 0.0 |
| | #12091 - Homo sapiens, 1219 aa. | 556 . . . 1013 | 457/458 (99%) | |
| | [WO200175067-A2, 11-OCT-2001] | | | |
| ABU52329 | Human GPCR related protein | 1 . . . 458 | 456/458 (99%) | 0.0 |
| | NOV12a - Homo sapiens, 1386 aa. | 508 . . . 965 | 456/458 (99%) | |
| | [WO200279398-A2, 10-OCT-2002] | | | |

In a BLAST search of public sequence databases, the NOV46a protein was found to have homology to the proteins shown in the BLASTP data in Table 46E.

TABLE 46E
Public BLASTP Results for NOV46a

| | | NOV46a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8NFP4 | Glycosyl-phosphatidyl-inositol-MAM - | 1 . . . 488 | 446/488 (91%) | 0.0 |
| | Homo sapiens (Human), 955 aa. | 508 . . . 955 | 446/488 (91%) | |
| Q8HYV1 | Glycosylphosphatidylinositol anchor 1 | 1 . . . 488 | 430/488 (88%) | 0.0 |
| | protein - Sus scrofa (Pig), 885 aa | 438 . . . 885 | 435/488 (89%) | |
| | (fragment). | | | |
| Q8HYV2 | MAM domain containing | 1 . . . 488 | 430/488 (88%) | 0.0 |
| | glycosylphosphatidylinositol anchor 1 - | 439 . . . 886 | 435/488 (89%) | |
| | Sus scrofa (Pig), 886 aa (fragment). | | | |
| CAB86653 | DJ402N21.2 (novel protein with MAM | 176 . . . 488 | 271/313 (86%) | e−156 |
| | domain) - Homo sapiens (Human), | 1 . . . 273 | 271/313 (86%) | |
| | 273 aa (fragment). | | | |
| Q8NBE3 | Hypothetical protein FLJ33358 - | 276 . . . 476 | 185/201 (92%) | e−108 |
| | Homo sapiens (Human), 242 aa. | 12 . . . 212 | 185/201 (92%) | |

PFam analysis predicts that the NOV46a protein contains the domains shown in the Table 46F.

TABLE 46F
Domain Analysis of NOV46a

| | | Identities/ | |
|---|---|---|---|
| Pfam | NOV46a | Similarities for | Expect |
| Domain | Match Region | the Matched Region | Value |
| ig | 46 . . . 110 | 17/66 (26%) | 0.0094 |
| | | 38/66 (58%) | |
| MAM | 286 . . . 451 | 78/185 (42%) | 6.1e−74 |
| | | 146/185 (79%) | |

### Example 47

TABLE 47A
NOV47 Sequence Analysis

| | | |
|---|---|---|
| NOV47a, CG57448-01 | SEQ ID NO: 1041 | 2907 bp |
| DNA Sequence | ORF Start: ATG at 10 | ORF Stop: TGA at 2896 |

GGCCGCTGAATGCTAGCTAGAATTCAGCGGCCGCTGAATTCTAGGCGCTGCCGAGGGAATGCGCGCAG
CTCACAGGCCCTGGGAGTGAGCTGGTGCCCGGCGACCTGGCACCCGCGCCTGGATATGGGGCGTCTAC
ATCGTCCCAGGAGCAGCACCAGCTACAGGAACCTGCCGCATCTGTTTCTGTTTTTCCTCTTCGTGGGA
CCCTTCAGCTGCCTCGGGAGTTACAGCCGGGCCACCGAGCTTCTGTACAGCCTAAACGAGGGACTACC
CGCGGGGGTGCTCATCGGCAGCCTGGCCGAGGACCTGCGGCTGCTGCCCAGGTCTGCAGGGAGGCCGG
ACCCGCAGTCGCAGCTGCCAGAGCGCACCGGTGCTGAGTGGAACCCCCCTCTCTCCTTCAGCCTGGCC
TCCCGGGGACTGAGTGGCCAGTACGTGACCCTAGACAACCGCTCTGGGGAGCTGCACACTTCAGCTCA
GGAGATCGACAGGGAGGCCCTGTGTGTTGAAGGGGGTGGAGGGACTGCGTGGAGCGGCAGCGTTTCCA
TCTCCTCCTCTCCTTCTGACTCTTGTCTTTTGCTGCTGGATGTGCTTGTCCTGCCTCAGGAATACTTC
AGGTTTGTGAAGGTGAAGATCGCCATCAGAGACATCAATGACAACGCCCCGCAGTTCCCTGTTTCCCA
GATCTCGGTGTGGGTCCCGGAAAATGCACCTGTAAACACCCGACTGGCCATAGAGCATCCTGCTGTGG
ACCCAGATGTAGGCATTAATGGGGTACAGACCTATCGCTTACTGGACTACCATGGTATGTTCACCCTG
GACGTGGAGGAGAATGAGAATGGGGAGCGCACCCCCTACCTAATTGTCATGGGTGCTTTGGACAGGGA
AACCCAGGACCAGTATGTGAGCATCATCATAGCTGAGGATGGTGGGTCTCCACCACTTTTGGGCAGTG
CCACTCTCACCATTGGCATCAGTGACATTAATGACAATTGCCCTCTCTTCACAGACTCACAAATCAAT
GTCACTGTGTATGGGAATGCTACAGTGGGCACCCCAATTGCAGCTGTCCAGGCTGTGGATAAAGACTT
GGGGACCAATGCTCAAATTACTTATTCTTACAGTCAGAAAGTTCCACAAGCATCTAAGGATTTATTTC
ACCTGGATGAAAACACTGGAGTCATTAAACTTTTCAGTAAGATTGGAGGAAGTGTTCTGGAGTCCCAC
AAGCTCACCATCCTTGCTAATGGACCAGGCTGCATCCCTGCTGTAATCACTGCTCTTGTGTCCATTAT
TAAAGTTATTTTCAGACCCCCTGAAATTGTCCCTCGTTACATAGCAAACGAGATAGATGGTGTTGTTT
ATCTGAAAGAACTGGAACCCGTTAACACTCCCATTGCGTTTTTCACCATAAGAGATCCAGAAGGTAAA
TACAAGGTTAACTGCTACCTGGATGGTGAAGGGCCGTTTAGGTTATCACCTTACAAACCATACAATAA
TGAATATTTACTAGAGACCACAAAACCTATGGACTATGAGCTACAGCAGTTCTATGAAGTAGCTGTGG
TGGCTTGGAACTCTGAGGGATTTCATGTCAAAAGGGTCATTAAAGTGCAACTTTTAGATGACAATGAT
AATGCTCCAATTTTCCTTCAACCCTTAATAGAACTAACCATCGAAGAGAACAACTCACCCAATGCCTT
TTTGACTAAGCTGTATGCTACAGATGCCGACAGCGAGGAGAGAGGCCAAGTTTCATATTTTCTGGGAC
CTGATGCTCCATCATATTTTTCCTTAGACAGTGTCACAGGAATTCTGACAGTTTCTACTCAGCTGGAC
CGAGAAGAGAAAGAAAAGTACAGATACACTGTCAGAGCTGTTGACTGTGGGAAGCCACCCAGAGAATC
AGTAGCCACTGTGGCCCTCACAGTGTTGGATAAAAATGACAACAGTCCTCGGTTTATCAACAAGGACT
TCAGCTTTTTTGTGCCTGAAAACTTTCCAGGCTATGGTGAGATTGGAGTAATTAGTGTAACAGATGCT
GACGCTGGACGAAATGGATGGGTCGCCCTCTCTGTGGTGAACCAGAGTGATATTTTTGTCATAGATAC
AGGAAAGGGTATGCTGAGGGCTAAAGTCTCTTTGGACAGAGAGCAGCAAAGCTCCTATACTTTGTGGG
TTGAAGCTGTTGATGGGGGTGAGCCTGCCCTCTCCTCTACAGCAAAAATCACAATTCTCCTTCTAGAT
ATCAATGACAACCCTCCTCTTGTTTTGTTTCCTCAGTCTAATATGTCTTATCTGTTAGTACTGCCTTC
TACTCTGCCAGGCTCCCCGGTTACAGAAGTCTATGCTGTCGACAAAGACACAGGCATGAATGCTGTCA
TAGCTTACAGCATCATAGGGAGAAGAGGTCCTAGGCCTGAGTCCTTCAGGATTGACCCTAAAACTGGC
AACATTACTTTGGAAGAGGCATTGCTGCAGACAGATTATGGGCTCCATCGCTTACTGGTGAAAGTGAG
TGATCATGGTTATCCCGAGCCTCTCCACTCCACAGTCATGGTGAACCTATTTGTCAATGACACTGTCA
GTAATGAGAGTTACATTGAGAGTCTTTTAAGAAAAGAACCAGAGATTAATATAGAGGAGAAAGAACCA
CAAATCTCAATAGAACCGACTCATAGGAAGGTAGAATCTGTGTCTTGTATGCCCACCTTAGTAGCTCT
GTCTGTAATAAGCTTGGGTTCCATCACACTGGTCACAGGGATGGGCATATACATCTGTTTAAGGAAAG
GGGAAAAGCATCCCAGGGAAGATGAAAATTTGGAAGTACAGATTCCACTGAAAGGAAAAATTGACTTG
CATATGCGAGAGAGAAAGCCAATGGATATTTCTAATATTTGATATTTCATG

| | | | |
|---|---|---|---|
| NOV47a, CG57448-01 | SEQ ID NO: 1042 | 962 aa | MW at 106269.4kD |
| Protein Sequence | | | |

MLARIQRPLNSRRCRGNARSSQALGVSWCPATWHPRLDMGRLHRPRSSTSYRNLPHLFLFFLFVGPFS
CLGSYSRATELLYSLNEGLPAGVLIGSLAEDLRLLPRSAGRPDPQSQLPERTGAEWNPPLSFSLASRG
LSGQYVTLDNRSGELHTSAQEIDREALCVEGGGGTAWSGSVSISSSPSDSCLLLLDVLVLPQEYFRFV
KVKIAIRDINDNAPQFPVSQISVWVPENAPVNTRLAIEHPAVDPDVGINGVQTYRLLDYHGMFTLDVE
ENENGERTPYLIVMGALDRETQDQYVSIIIAEDGGSPPLLGSATLTIGISDINDNCPLFTDSQINVTV
YGNATVGTPIAAVQAVDKDLGTNAQITYSYSQKVPQASKDLFHLDENTGVIKLFSKIGGSVLESHKLT
ILANGPGCIPAVITALVSIIKVIFRPPEIVPRYIANEIDGVVYLKELEPVNTPIAFFTIRDPEGKYKV
NCYLDGEGPFRLSPYKPYNNEYLLETTKPMDYELQQFYEVAVVAWNSEGFHVKRVIKVQLLDDNDNAP
IFLQPLIELTIEENNSPNAFLTKLYATDADSEERGQVSYFLGPDAPSYFSLDSVTGILTVSTQLDREE
KEKYRYTVPAVDCGKPPRESVATVALTVLDKNDNSPRFINKDFSFFVPENFPGYGEIGVISVTDADAG
RNGWVALSVVNQSDIFVIDTGKGMLRAKVSLDREQQSSYTLWVEAVDGGEPALSSTAKITILLLDIND
NPPLVLFPQSNMSYLLVLPSTLPGSPVTEVYAVDKDTGMNAVIAYSIIGRRGPRPESFRIDPKTGNIT
LEEALLQTDYGLHRLLVKVSDHGYPEPLHSTVMVNLFVNDTVSNESYIESLLRKEPEINIEEKEPQIS
IEPTHRKVESVSCMPTLVALSVISLGSITLVTGMGIYICLRKGEKHPREDENLEVQIPLKGKIDLHMR
ERKPMDISNI

| | | |
|---|---|---|
| NOV47b, 247846705 | SEQ ID NO: 1043 | 1377 bp |
| DNA Sequence | ORF Start: at 1 | ORF Stop: end of sequence |

GGATCCCCTCGTTACATAGCAAACGAGATAGATGGTGTTGTTTATCTGAAAGAACTGGAACCCGTTAA
CACTCCCATTGCGTTTTTCACCATAAGAGATCCAGAAGGTAAATACAAGGTTAACTGCTACCTGGATG
GTGAAGGGCCGTTTAGGTTATCACCTTACAAACCATACAATAATGAATATTTACTAGAGACCACAAAA
CCTATGGACTATGAGCTACAGCAGTTCTATGAAGTAGCTGTGGTGGCTTGGAACTCTGAGGGATTTCA
TGTCAAAAGGGTCATTAAAGTGCAACTTTTAGATGACAATGATAATGCTCCAATTTTCCTTCAACCCT
TAATAGACTAACCATCGAAGAGAACAACTCACCCAATGCCTTTTTGACTAAGGTGTATGCTACAGAT
GCCGACAGCGAGGAGAGAGGCCAAGTTTCATATTTTCTGGGACCTGATGCTCCATCATATTTTTCCTT
AGACAGTGTCACAGGAATTCTGACAGTTTCTACTCAGCTGGACCGAGAAGAGAAAGAAAAGTACAGAT
ACACTGTCAGAGCTGTTGACTGTGGGAAGCCACCCAGAGAATCAGTAGCCACTGTGGCCCTCACAGTG
TTGGATAAAAATGACAACAGTCCTCGGTTTATCAACAAGGACTTCAGCTTTTTTGTGCCTGAAAACTT
TCCAGGCTATGGTGAGATTGGAGTAATTAGTGTAACAGATGCTGACGCTGGACGAAATGGATGGGTCG
CCCTCTCTGTGGTGAACCAGAGTGATATTTTTGTCATAGATACAGGAAAGGGTATGCTGAGGGCTAAA
GTCTCTTTGGACAGAGAGCAGCAAAGCTCCTATACTTTGTGGGTTGAAGCTGTTGATGGGGGTGAGCC
TGCCCTCTCCTCTACAGCAAAAATCACAATTCTCCTTCTAGATATCAATGACAACCCTCCTCTTGTTT
TGTTTCCTCAGTCTAATATGTCTTATCTGTTAGTACTGCCTTCTACTCTGCCAGGCTCCCCGGTTACA
GAAGTCTATGCTGTCGACAAAGACACAGGCATGAATGCTGTCATAGCTTACAGCATCATAGGGAGAAG
AGGTCCTAGGCCTGAGTCCTTCAGGATTGACCCTAAAACTGGCAACATTACTTTGGAAGAGGCATTGC
TGCAGACAGATTATGGGCTCCATCGCTTACTGGTGAAAGTGAGTGATCATGGTTATCCCGAGCCTCTC
CACTCCACAGTCATGGTGAACCTATTTGTCAATGACACTGTCAGTAATGAGAGTTACATTGAGAGTCT
TTTAAGAAAAGAACCAGAGATTAATATAGAGGAGAAAGAACCACAAATCTCAATAGAACCGACTCATA
GGAAGGTAGAACTCGAG

| | | | |
|---|---|---|---|
| NOV47b, 247846705 | SEQ ID NO: 1044 | 459 aa | MW at 51423.4kD |
| Protein Sequence | | | |

GSPRYIANEIDGVVYLKELEPVNTPIAFFTIRDPEGKYKVNCYLDGEGPFRLSPYKPYNNEYLLETTK
PMDYELQQFYEVAVVAWNSEGFHVKRVIKVQLLDDNDNAPIFLQPLIELTIEENNSPNAFLTKVYATD
ADSEERGQVSYFLGPDAPSYFSLDSVTGILTVSTQLDREEKEKYRYTVPAVDCGKPPRESVATVALTV
LDKNDNSPRFINKDFSFFVPENFPGYGEIGVISVTDADAGRNGWVALSVVNQSDIFVIDTGKGMLRAK
VSLDREQQSSYTLWVEAVDGGEPALSSTAKITILLLDINDNPPLVLFPQSNMSYLLVLPSTLPGSPVT
EVYAVDKDTGMNAVIAYSIIGRRGPRPESFRIDPKTGNITLEEALLQTDYGLHRLLVKVSDHGYPEPL
HSTVMVNLFVNDTVSNESYIESLLRKEPEINIEEKEPQISIEPTHRKVELE

| | | |
|---|---|---|
| NOV47c, 247846708 | SEQ ID NO: 1045 | 1377 bp |
| DNA Sequence | ORF Start: at 1 | ORF Stop: end of sequence |

GGATCCCCTCGTTACATAGCAAACGAGATAGATGGTGTGGTTTATCTGAAAGAACTGGAACCCGTTAA
CACTCCCATTGCGTTTTTCACCATAAGAGATCCAGAAGGTAAATACAAGGTTAACTGCTACCTGGATG
GTGAAGGGCCGTTTAGGTTATCACCTTACAAACCATACAATAATGAATATTTACTAGAGACCACAAAA
CCTATGGACTATGAGCTACAGCAGTTCTATGAAGTAGCTGTGGTGGCTTGGAACTCTGAGGGATTTCA
TGTCAAAAGGGTCATTAAAGTGCAACTTTTAGATGACAATGATAATGCTCCAATTTTCCTTCAACCCT
TAATAGAACTAACCATCGAAGAGAACAACTCACCCAATGCCTTTTTGACTAAGCTGTATGCTACAGAT
GCCGACAGCGAGGAGAGAGGCCAAGTTTCATATTTTCTGGGACCTGATGCTCCATCATATTTTTCCTT
AGACAGTGTCACAGGAATTCTGACAGTTTCTACTCAGCTGGACCGAGAAGAGAAAGAAAAGTACAGAT
ACACTGTCAGAGCTGTTGACTGTGGGAAGCCACCCAGAGAATCAGTAGCCACTGTGGCCCTCACAGTG
TTGGATAAAAATGACAACAGTCCTCGGTTTATCAACAAGGACTTCAGCTTTTTTGTGCCTGAAAACTT
TCCAGGCTATGGTGAGATTGGAGTAATTAGTGTAACAGATGCTGACGCTGGACGAAATGGATGGGTCG
CCCTCTCTGTGGTGAACCAGAGTGATATTTTTGTCATAGATACAGGAAAGGGTATGCTGAGGGCTAAA
GTCTCTTTGGACAGAGAGCAGCAAAGCTCCTATACTTTGTGGGTTGAAGCTGTTGATGGGGGTGAGCC
TGCCCTCTCCTCTACAGCAAAAATCACAATTCTCCTTCTAGATATCAATGACAACCCTCCTCTTGTTT
TGTTTCCTCAGTCTAATATGTCTTATCTGTTAGTACTGCCTTCTACTCTGCTAGGCTCCCCGGTTACA
GAAGTCTATGCTGTCGACAAAGACACAGGCATGAATGCTGTCATAGCTTACAGCATCATAGGGAGAAG
AGGTCCTAGGCCTGAGTCCTTCAGGATTGACCCTAAAACTGGCAACATTACTTTGGAAGAGGCATTGC
TGCAGACAGATTATGGGCTCCATCGCTTACTGGTGAAAGTGAGTGATCATGGTTATCCCGAGCCTCTC
CACTCCACAGTCATGGTGAACCTATTTGTCAATGACACTGTCAGTAATGAGAGTTACATTGAGAGTCT
TTTAAGAAAAGAACCAGAGATTAATATAGAGGAGAAAGAACCACAAATCTCAATAGAACCGACTCATA
GGAAGGTAGAACTCGAG

| | | | |
|---|---|---|---|
| NOV47c, 247846708 | SEQ ID NO: 1046 | 459 aa | MW at 51453.4kD |
| Protein Sequence | | | |

GSPRYIANEIDGVVYLKELEPVNTPIAFFTIRDPEGKYKVNCYLDGEGPFRLSPYKPYNNEYLLETTK
PMDYELQQFYEVAVVAWNSEGFHVKRVIKVQLLDDNDNAPIFLQPLIELTIEENNSPNAFLTKLYATD
ADSEERGQVSYFLGPDAPSYFSLDSVTGILTVSTQLDREEKEKYRYTVPAVDCGKPPRESVATVALTV
LDKNDNSPRFINKDFSFFVPENFPGYGEIGVISVTDADAGRNGWVALSVVNQSDIFVIDTGKGMLRAK
VSLDREQQSSYTLWVEAVDGGEPALSSTAKITILLLDINDNPPLVLFPQSNMSYLLVLPSTLLGSPVT
EVYAVDKDTGMNAVIAYSIIGRRGPRPESFRIDPKTGNITLEEALLQTDYGLHRLLVKVSDHGYPEPL
HSTVMVNLFVNDTVSNESYIESLLRKEPEINIEEKEPQISIEPTHRKVELE

| | | |
|---|---|---|
| NOV47d, 237580295 | SEQ ID NO: 1047 | 1377 bp |
| DNA Sequence | ORF Start: at 1 | ORF Stop: end of sequence |

GGATCCCCTCGTTACATAGCAAACGAGATAGATGGTGTTGTTTATCTGAAAGAACTGGAACCCGTTAA
CACTCCCATTGCGCTTTTCACCATAAGAGATCCAGAAGGTAAATACAAGGTTAACTGCTACCTGGATG
GTGAAGGGCCGTTTAGGTTATCACCTTACAAACCATACAATAATGAATATTTACTAGAGACCACAAAA
CCTATGGACTATGAGCTACAGCAGTTCTATGAAGTAGCTGTGGTGGCTTGGAACTCTGAGGGATTTCA
TGTCAAAAGGGTCATTAAAGTGCAACTTTTAGATGACAATGATAATGCTCCAATTTTCCTTCAACCCT
TAATAGAACTAACCATCGAAGAGAACAACTCACCCAATGCCTTTTTGACTAAGCTGTATGCTACAGAT
GCCGACAGCGAGGAGAGAGGCCAAGTTTCATATTTTCTGGGACCTGATGCTCCATCATATTTTTCCTT
AGACAGTGTCACAGGAATTCTGACAGTTTCTACTCAGCTGGACCGAGAAGAGAAAGAAAAGTACAGAT
ACACTGTCAGAGCTGTTGACTGTGGGAAGCCACCCAGAGAATCAGTAGCCACTGTGGCCCTCACAGTG
TTGGATAAAAATGACAACAGTCCTCGGTTTATCAACAAGGACTTCAGCTTTTTTGTGCCTGAAAACTT
TCCAGGCTATGGTGAGATTGGAGTAATTAGTGTAACAGATGCTGACGCTGGACGAAATGGATGGGTCG
CCCTCTCTGTGGTGAACCAGAGTGATATTTTTGTCATAGATACAGGAAAGGGTATGCTGAGGGCTAAA
GTCTCTTTGGACAGAGAGCAGCAAAGCTCCTATACTTTGTGGGTTGAAGCTGTTGATGGGGGTGAGCC
TGCCCTCTCCTCTACAGCAAAAATCACAATTCTCCTTCTAGATATCAATGACAACCCTCCTCTTGTTT
TGTTTCCTCAGTCTAATATGTCTTATCTGTTAGTACTGCCTTCTACTCTGCCAGGCTCCCCGGTTACA
GAAGTCTATGCTGTCGACAAAGACACAGGCATGAATGCTGTCATAGCTTACAGCATCATAGGGAGAAG
AGGTCCTAGGCCTGAGTCCTTCAGGATTGACCCTAAAACTGGCAACATTACTTTGGAAGAGGCATTGC
TGCAGACAGATTATGGGCTCCATCGCTTACTGGTGAAAGTGAGTGATCATGGTTATCCCGAGCCTCTC
CACTCCACAGTCATGGTGAACCTATTTGTCAATGACACTGTCAGTAATGAGAGTTACATTGAGAGTCT
TTTAAGAAAAGAACCAGAGATTAATATAGAGGAGAAAGAACCACAAATCTCAATAGAACCGACTCATA
GGAAGGTAGAACTCGAG

| | | | |
|---|---|---|---|
| NOV47d, 237580295 | SEQ ID NO: 1048 | 459 aa | MW at 51403.4kD |
| Protein Sequence | | | |

GSPRYIANEIDGVVYLKELEPVNTPIALFTIRDPEGKYKVNCYLDGEGPFRLSPYKPYNNEYLLETTK
PMDYELQQFYEVAVVAWNSEGFHVKRVIKVQLLDDNDNAPIFLQPLIELTIEENNSPNAFLTKLYATD
ADSEERGQVSYFLGPDAPSYFSLDSVTGILTVSTQLDREEKEKYRYTVRAVDCGKPPRESVATVALTV
LDKNDNSPRFINKDFSFFVPENFPGYGEIGVISVTDADAGRNGWVALSVVNQSDIFVIDTGKGMLRAK
VSLDREQQSSYTLWVEAVDGGEPALSSTAKITILLLDINDNPPLVLFPQSNMSYLLVLPSTLPGSPVT
EVYAVDKDTGMNAVIAYSIIGRRGPRPESFRIDPKTGNITLEEALLQTDYGLHRLLVKVSDHGYPEPL
HSTVMVNLFVNDTVSNESYIESLLRKEPEINIEEKEPQISIEPTHRKVELE

| | | |
|---|---|---|
| NOV47e, 237579512 | SEQ ID NO: 1049 | 1377 bp |
| DNA Sequence | ORF Start: at 1 | ORF Stop: end of sequence |

GGATCCCCTCGTTACATAGCAAACGAGATAGATGGTGTGGTTTATCTGAAAGAACTGGAACCCGTTAA
CACTCCCATTGCGTTTTTCACCATAAGAGATCCAGAAGGTAAATACAAGGTTAACTGCTACCTGGATG
GTGAAGGGCCGTTTAGGTTATCACCTTACAAACCATACAATAATGAATATTTACTAGAGACCACAAAA
CCTATGGACTATGAGCTACAGCAGTTCTATGAAGTAGCTGTGGTGGCTTGGAACTCTGAGGGATTTCA
TGTCAAAAGGGTCATTAAAGTGCAACTTTTAGATGACAATGATAATGCTCCAATTTTCCTTCAACCCT
TAATAGAACTAACCATCGAAGAGAACAACTCACCCAATGCCTTTTTGACTAAGCTGTATGCTACAGAT
GCCGACAGCGAGGAGAGAGGCCAAGTTTCATATTTTCTGGGACCTGATGCTCCATCATATTTTTCCTT
AGACAGTGTCACAGGAATTCTGACAGTTTCTACTCAGCTGGACCGAGAAGAGAAAGAAAAGTACAGAT
ACACTGTCAGAGCTGTTGACTGTGGGAAGCCACCCAGAGAATCAGTAGCCACTGTGGCCCTCACAGTG
TTGGATAAAAATGACAACAGTCCTCGGTTTATCAACAAGGACTTCAGCTTTTTTGTGCCTGAAAACTT
TCCAGGCTATGGTGAGATTGGAGTAATTAGTGTAACAGATGCTGACGCTGGACGAAATGGATGGGTCG
CCCTCTCTGTGGTGAACCAGAGTGATATTTTTGTCATAGATACAGGAAAGGGTATGCTGAGGGCTAAA
GTCTCTTTGGACAGAGAGCAGCA)AGCTCCTATACTTTGTGGGTTGAAGCTGTTGATGGGGGTGAGCC
TGCCCTCTCCTCTACAGCAAAAATCACAATTCTCCTTCTAGATATCAATGACAACCCTCCTCTTGTTT
TGTTTCCTCAGTCTAATATGTCTTATCTGTTAGTACTGCCTTCTACTCTGCCAGGCTCCCCGGTTACA
GAAGTCTATGCTGTCGACAAAGACACAGGCATGAATGCTGTCATAGCTTACAGCATCATAGGGAGAAG
AGGTCCTAGGCCTGAGTCCTTCAGGATTGACCCTAAAACTGGCAACATTACTTTGGAAGAGGCATTGC
TGCAGACAGATTATGGGCTCCATCGCTTACTGGTGAAAGTGAGTGATCATGGTTATCCCGAGCCTCTC
CACTCCACAGTCATGGTGAACCTATTTGTCAATGACACTGTCAGTAATGAGAGTTACATTGAGAGTCT
TTTAAGAAAAGAACCAGAGATTAATATAGAGGAGAAAGAACCACAAATCTCAATAGAACCGACTCATA
GGAAGGTAGAACTCGAG

| | | | |
|---|---|---|---|
| NOV47e, 237579512 | SEQ ID NO: 1050 | 459 aa | MW at 51437.4kD |
| Protein Sequence | | | |

GSPRYIANEIDGVVYLKELEPVNTPIAFFTIRDPEGKYKVNCYLDGEGPFRLSPYKPYNNEYLLETTK
PMDYELQQFYEVAVVAWNSEGFHVKRVIKVQLLDDNDNAPIFLQPLIELTIEENNSPNAFLTKLYATD
ADSEERGQVSYFLGPDAPSYFSLDSVTGILTVSTQLDREEKEKYRYTVEAVDCGKPPRESVATVALTV
LDKNDNSPRFINKDFSFFVPENFPGYGEIGVISVTDADAGRNGWVALSVVNQSDIFVIDTGKGMLRAK
VSLDREQQSSYTLWVEAVDGGEPALSSTAKITILLLDINDNPPLVLFPQSNMSYLLVLPSTLPGSPVT
EVYAVDKDTGHNAVIAYSIIGRRGPRPESFRIDPKTGNITLEEALLQTDYGLHRLLVKVSDHGYPEPL
HSTVMVNLFVNDTVSNESYIESLLRKEPEINIEEKEPQISIEPTHRKVELE

| | | |
|---|---|---|
| NOV47f, CG57448-02 | SEQ ID NO: 1051 | 576 bp |
| DNA Sequence | ORF Start: at 7 | ORF Stop: at 571 |

GGATCCATCTCGGTGTGGGTCCCGGAAAATGCACCTGTAAACACCCGACTGGCCATAGAGCATCCTGC
TGTGGACCCAGATGTAGGCATTAATGGGGTACAGACCTATCGCTTACTGGACTACCATGGTATGTTCA
CCCTGGACGTGGAGGAGAATGAGAATGGGGAGCGCACCCCCTACCTAATTGTCATGGGTGCTTTGGAC
AGGGAAACCCAGGACCAGTATGTGAGCATCATCATAGCTGAGGATGGTGGGTCTCCACCACTTTTGGG
CAGTGCCACTCTCACCATTGGCATCAGTGACATTAATGACAATTGCCCTCTCTTCACAGACTCACAAA
TCAATGTCACTGTGTATGGGAATGCTACAGTGGGCACCCCAATTGCAGCTGTCCAGGCTGTGGATAAA
GACTTGGGGACCAATGCTCAAATTACTTATTCTTACAGTCAGAAAGTTCCACAAGCATCTAAGGATTT
ATTTCACCTGGATGAAAACACTGGAGTCATTAAACTTTTCAGTAAGATTGGAGGAAGTGTTCTGGAGT
CCCACAAGCTCACCATCCTTGCTAATCTCGAG

| | | | |
|---|---|---|---|
| NOV47f, CG57448-02 | SEQ ID NO: 1052 | 188 aa | MW at 20271.5kD |
| Protein Sequence | | | |

ISVWVPENAPVNTRLAIEHPAVDPDVGINGVQTYRLLDYHGMFTLDVEENENGERTPYLIVMGALDRE
TQDQYVSIIIAEDGGSPPLLGSATLTIGISDINDNCPLFTDSQINVTVYGNATVGTPIAAVQAVDKDL
GTNAQITYSYSQKVPQASKDLFHLDENTGVIKLFSKIGGSVLESHKLTILAN

A ClustalW comparison of the above protein sequences yields the following sequence alignment shown in Table 47B.

TABLE 47B
Comparison of the NOV47 protein sequences.

| | |
|---|---|
| NOV47a | MLARIQRPLNSRRCRGNARSSQALGVSWCPATWHPRLDMGRLHRPRSSTSYRNLPHLFLF |
| NOV47b | ------------------------------------------------------------ |
| NOV47c | ------------------------------------------------------------ |
| NOV47d | ------------------------------------------------------------ |
| NOV47e | ------------------------------------------------------------ |
| NOV47f | ------------------------------------------------------------ |
| NOV47a | FLFVGPFSCLGSYSRATELLYSLNEGLPAGVLIGSLAEDLRLLPRSAGRPDPQSQLPERT |
| NOV47b | ------------------------------------------------------------ |
| NOV47c | ------------------------------------------------------------ |
| NOV47d | ------------------------------------------------------------ |
| NOV47e | ------------------------------------------------------------ |
| NOV47f | ------------------------------------------------------------ |
| NOV47a | GAEWNPPLSFSLASRGLSGQYVTLDNRSGELHTSAQEIDREALCVEGGGGTAWSGSVSIS |
| NOV47b | ------------------------------------------------------------ |
| NOV47c | ------------------------------------------------------------ |
| NOV47d | ------------------------------------------------------------ |
| NOV47e | ------------------------------------------------------------ |
| NOV47f | ------------------------------------------------------------ |
| NOV47a | SSPSDSCLLLLDVLVLPQEYFRFVKVKIAIRDINDNAPQFPVSQISVWVPENAPVNTRLA |
| NOV47b | ------------------------------------------------------------ |
| NOV47c | ------------------------------------------------------------ |
| NOV47d | ------------------------------------------------------------ |
| NOV47e | ------------------------------------------------------------ |
| NOV47f | ------------------------------------------------------------ |
| NOV47a | IEHPAVDPDVGINGVQTYRLLDYHGMFTLDVEENENGERTPYLIVMGALDRETQDQYVSI |
| NOV47b | ------------------------------------------------------------ |
| NOV47c | ------------------------------------------------------------ |
| NOV47d | ------------------------------------------------------------ |
| NOV47e | ------------------------------------------------------------ |
| NOV47f | ------------------------------------------------------------ |
| NOV47a | IIAEDGGSPPLLGSATLTIGISDINDNCPLFTDSQINVTVYGNATVGTPIAAVQAVDKDL |
| NOV47b | ------------------------------------------------------------ |
| NOV47c | ------------------------------------------------------------ |
| NOV47d | ------------------------------------------------------------ |
| NOV47e | ------------------------------------------------------------ |
| NOV47f | ------------------------------------------------------------ |
| NOV47a | GTNAQITYSYSQKVPQASKDLFHLDENTGVIKLFSKIGGSVLESHKLTILANGPGCIPAV |
| NOV47b | ------------------------------------------------------------ |
| NOV47c | ------------------------------------------------------------ |
| NOV47d | ------------------------------------------------------------ |
| NOV47e | ------------------------------------------------------------ |
| NOV47f | ------------------------------------------------------------ |
| NOV47a | ITALVSIIKVIFRPPEIVPRYIANEIDGVVYLKELEPVNTPIAFFTIRDPEGKYKVNCYL |
| NOV47b | ----------------GSPRYIANEIDGVVYLKELEPVNTPIAFFTIRDPEGKYKVNCYL |
| NOV47c | ----------------GSPRYIANEIDGVVYLKELEPVNTPIAFFTIRDPEGKYKVNCYL |
| NOV47d | ----------------GSPRYIANEIDGVVYLKELEPVNTPIALFTIRDPEGKYKVNCYL |
| NOV47e | ----------------GSPRYIANEIDGVVYLKELEPVNTPIAFFTIRDPEGKYKVNCYL |
| NOV47f | ------------------------------------------------------------ |
| NOV47a | DGEGPFRLSPYKPYNNEYLLETTKPMDYELQQFYEVAVVAWNSEGFHVKRVIKVQLLDDN |
| NOV47b | DGEGPFRLSPYKPYNNEYLLETTKPMDYELQQFYEVAVVAWNSEGFHVKRVIKVQLLDDN |
| NOV47c | DGEGPFRLSPYKPYNNEYLLETTKPMDYELQQFYEVAVVAWNSEGFHVKRVIKVQLLDDN |
| NOV47d | DGEGPFRLSPYKPYNNEYLLETTKPMDYELQQFYEVAVVAWNSEGFHVKRVIKVQLLDDN |
| NOV47e | DGEGPFRLSPYKPYNNEYLLETTKPMDYELQQFYEVAVVAWNSEGFHVKRVIKVQLLDDN |
| NOV47f | ------------------------------------------------------------ |
| NOV47a | DNAPIFLQPLIELTIEENNSPNAFLTKLYATDADSEERGQVSYFLGPDAPSYFSLDSVTG |
| NOV47b | DNAPIFLQPLIELTIEENNSPNAFLTKVYATDADSEERGQVSYFLGPDAPSYFSLDSVTG |
| NOV47c | DNAPIFLQPLIELTIEENNSPNAFLTKLYATDADSEERGQVSYFLGPDAPSYFSLDSVTG |
| NOV47d | DNAPIFLQPLIELTIEENNSPNAFLTKLYATDADSEERGQVSYFLGPDAPSYFSLDSVTG |
| NOV47e | DNAPIFLQPLIELTIEENNSPNAFLTKLYATDADSEERGQVSYFLGPDAPSYFSLDSVTG |
| NOV47f | ------------------------------------------------------------ |
| NOV47a | ILTVSTQLDREEKEKYRYTVRAVDCGKPPRESVATVALTVLDKNDNSPRFINKDFSFFVP |
| NOV47b | ILTVSTQLDREEKEKYRYTVRAVDCGKPPRESVATVALTVLDKNDNSPRFINKDFSFFVP |
| NOV47c | ILTVSTQLDREEKEKYRYTVRAVDCGKPPRESVATVALTVLDKNDNSPRFINKDFSFFVP |
| NOV47d | ILTVSTQLDREEKEKYRYTVRAVDCGKPPRESVATVALTVLDKNDNSPRFINKDFSFFVP |
| NOV47e | ILTVSTQLDREEKEKYRYTVRAVDCGKPPRESVATVALTVLDKNDNSPRFINKDFSFFVP |
| NOV47f | ------------------------------------------------------ISVWVP |
| NOV47a | ENFPGYGEIGVIS-VTDADAGRNGWVALSVVNQSDIFVIDTGKG-------MLRAKVSLD |
| NOV47b | ENFPGYGEIGVIS-VTDADAGRNGWVALSVVNQSDIFVIDTGKG-------MLRAKVSLD |
| NOV47c | ENFPGYGEIGVIS-VTDADAGRNGWVALSVVNQSDIFVIDTGKG-------MLRAKVSLD |
| NOV47d | ENFPGYGEIGVIS-VTDADAGRNGWVALSVVNQSDIFVIDTGKG-------MLRAKVSLD |
| NOV47e | ENFPGYGEIGVIS-VTDADAGRNGWVALSVVNQSDIFVIDTGKG-------MLRAKVSLD |
| NOV47f | ENAPVNTRLAIEHPAVDPDVGINGVQTYRLLDYHGMFTLDVEENENGERTPYLIVMGALD |
| NOV47a | REQQSSYTLWVEAVDGGEPALSSTAKITILLLDINDNPPLVLFPQSNMSYLLVLPSTLPG |
| NOV47b | REQQSSYTLWVEAVDGGEPALSSTAKITILLLDINDNPPLVLFPQSNMSYLLVLPSTLPG |
| NOV47c | REQQSSYTLWVEAVDGGEPALSSTAKITILLLDINDNPPLVLFPQSNMSYLLVLPSTLLG |
| NOV47d | REQQSSYTLWVEAVDGGEPALSSTAKITILLLDINDNPPLVLFPQSNMSYLLVLPSTLPG |
| NOV47e | REQQSSYTLWVEAVDGGEPALSSTAKITILLLDINDNPPLVLFPQSNMSYLLVLPSTLPG |
| NOV47f | RETQDQYVSIIIAEDGGSPPLLGSATLTIGISDINDNCPLFTDSQ--IN-VTVYGNATVG |
| NOV47a | SPVTEVYAVDKDTGMNAVIAYSIIGRR-GPRPESFRIDPKTGNITLEEALLQTDYGLHRL |
| NOV47b | SPVTEVYAVDKDTGMNAVIAYSIIGRR-GPRPESFRIDPKTGNITLEEALLQTDYGLHRL |
| NOV47c | SPVTEVYAVDKDTGMNAVIAYSIIGRR-GPRPESFRIDPKTGNITLEEALLQTDYGLHRL |
| NOV47d | SPVTEVYAVDKDTGMNAVIAYSIIGRR-GPRPESFRIDPKTGNITLEEALLQTDYGLHRL |
| NOV47e | SPVTEVYAVDKDTGMNAVIAYSIIGRR-GPRPESFRIDPKTGNITLEEALLQTDYGLHRL |
| NOV47f | TPIAAVQAVDKDLGTNAQITYSYSQKVPQASKDLFHLDENTGVIKLFSKIGGSVLESHKL |
| NOV47a | LVKVSDHGYPEPLHSTVMVNLFVNDTVSNESYIESLLRKEPEINIEEKEPQISIEPTHRK |
| NOV47b | LVKVSDHGYPEPLHSTVMVNLFVNDTVSNESYIESLLRKEPEINIEEKEPQISIEPTHRK |
| NOV47c | LVKVSDHGYPEPLHSTVMVNLFVNDTVSNESYIESLLRKEPEINIEEKEPQISIEPTHRK |
| NOV47d | LVKVSDHGYPEPLHSTVMVNLFVNDTVSNESYIESLLRKEPEINIEEKEPQISIEPTHRK |
| NOV47e | LVKVSDHGYPEPLHSTVMVNLFVNDTVSNESYIESLLRKEPEINIEEKEPQISIEPTHRK |
| NOV47f | TILAN------------------------------------------------------- |
| NOV47a | VESVSCMPTLVALSVISLGSITLVTGMGIYICLRKGEKHPREDENLEVQIPLKGKIDLHM |
| NOV47b | VELE-------------------------------------------------------- |
| NOV47c | VELE-------------------------------------------------------- |
| NOV47d | VELE-------------------------------------------------------- |
| NOV47e | VELE-------------------------------------------------------- |
| NOV47f | ------------------------------------------------------------ |
| NOV47a | RERKPMDISNI |
| NOV47b | ----------- |
| NOV47c | ----------- |
| NOV47d | ----------- |
| NOV47e | ----------- |
| NOV47f | ----------- |
| NOV47a | (SEQ ID NO: 1042) |
| NOV47b | (SEQ ID NO: 1044) |
| NOV47c | (SEQ ID NO: 1046) |
| NOV47d | (SEQ ID NO: 1048) |
| NOV47e | (SEQ ID NO: 1050) |
| NOV47f | (SEQ ID NO: 1052) |

Further analysis of the NOV47a protein yielded the following properties shown in Table 47C.

TABLE 47C
Protein Sequence Properties NOV47a

| | |
|---|---|
| SignalP analysis: | Cleavage site between residues 14 and 15 |
| PSORT II analysis: | |

| PSG: | a new signal peptide prediction method | |
|---|---|---|
| | N-region: | length 7; pos. chg 2; neg. chg 0 |
| | H-region: | length 4; peak value −19.10 |
| | PSG score: | −23.50 |

| | |
|---|---|
| GvH: | von Heijne's method for signal seq. recognition |
| | GvH score (threshold: −2.1): −9.89 |
| | possible cleavage site: between 29 and 30 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 3 | | |
| | INTEGRAL | Likelihood = | Transmembrane 54-70 |
| | | −2.76 | |
| | INTEGRAL | Likelihood = | Transmembrane 416-432 |
| | | −7.38 | |
| | INTEGRAL | Likelihood = | Transmembrane 901-917 |
| | | −3.77 | |

| | |
|---|---|
| PERIPHERAL | Likelihood = 2.01 (at 188) |

| | |
|---|---|
| | ALOM score: −7.38 (number of TMSs: 3) |
| MTOP: | Prediction of membrane topology (Hartmann et al.) |
| | Center position for calculation: 61 |
| | Charge difference: −5.0 C(−1.0)-N(4.0) |
| | N >= C: N-terminal side will be inside |

>>> membrane topology: type 3a
MITDISC: discrimination of mitochondrial targeting seq

| | | | |
|---|---|---|---|
| R content: | 7 | Hyd Moment(75): | 5.23 |
| Hyd Moment(95): | 11.82 | G content: | 2 |
| D/E content: | 1 | S/T content: | 5 |

Score: 2.50

Gavel: prediction of cleavage sites for mitochondrial preseq

R-2 motif at 85 SRA|TE

NUCDISC: discrimination of nuclear localization signals

| | |
|---|---|
| | pat4: none |
| | pat7: none |
| | bipartite: none |
| | content of basic residues: 8.9% |
| | NLS Score: −0.47 |
| KDEL: | ER retention motif in the C-terminus: none |

ER Membrane Retention Signals:

| | |
|---|---|
| | XXRR-like motif in the N-terminus: LARI |
| none | |

SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

| | |
|---|---|
| | type 1: none |
| | type 2: none |
| NMYR: | N-myristoylation pattern: none |

Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 76.7 |
| COIL: | Lupas's algorithm to detect coiled-coil regions |
| | total: 0 residues |

Final Results (k = 9/23):

```
34.8%: mitochondrial
34.8%: endoplasmic reticulum
17.4%: nuclear
  4.3%: cytoplasmic
  4.3%: plasma membrane
  4.3%: vesicles of secretory system
```

>> prediction for CG57448-01 is mit (k = 23)

A search of the NOV47a protein against the Geneseq database, a proprietary database that contains sequences published in patents and patent publication, yielded several homologous proteins shown in Table 47D.

TABLE 47D
Geneseq Results for NOV47a

| | | NOV47a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABU52605 | Human NOVX protein, NOV16a - | 1 . . . 962 | 962/962 (100%) | 0.0 |
| | Homo sapiens, 962 aa. | 1 . . . 962 | 962/962 (100%) | |
| | [WO200281518-A2, 17-OCT-2002] | | | |
| ABP70119 | Human NOV32a - Homo sapiens, 947 | 16 . . . 962 | 946/947 (99%) | 0.0 |
| | aa. [WO200272771-A2, 19-SEP-2002] | 1 . . . 947 | 946/947 (99%) | |
| AAU09958 | Human cadherin-1 (CDHN-1) protein - | 39 . . . 962 | 924/924 (100%) | 0.0 |
| | Homo sapiens, 924 aa. | 1 . . . 924 | 924/924 (100%) | |
| | [WO200179293-A2, 25-OCT-2001] | | | |
| ABP47865 | Human polypeptide SEQ ID NO 295 - | 566 . . . 962 | 396/397 (99%) | 0.0 |
| | Homo sapiens, 397 aa. | 1 . . . 397 | 397/397 (99%) | |
| | [US2002042386-A1, 11-APR-2002] | | | |
| AAU19645 | Human novel extracellular matrix | 566 . . . 962 | 396/397 (99%) | 0.0 |
| | protein, Seq ID No 295 - Homo | 1 . . . 397 | 397/397 (99%) | |
| | sapiens, 397 aa. [WO200155368-A1, | | | |
| | 02-AUG-2001] | | | |

In a BLAST search of public sequence databases, the NOV47a protein was found to have homology to the proteins shown in the BLASTP data in Table 47E.

TABLE 47E
Public BLASTP Results for NOV47a

| | | NOV47a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8NDN4 | Hypothetical protein - Homo | 13 . . . 962 | 949/950 (99%) | 0.0 |
| | sapiens (Human), 951 aa. | 2 . . . 951 | 949/950 (99%) | |
| Q9NRT9 | Protocadherin 13 - Homo sapiens | 16 . . . 962 | 946/947 (99%) | 0.0 |
| | (Human), 947 aa (fragment). | 1 . . . 947 | 946/947 (99%) | |
| Q8N6Y1 | Protocadherin 20 - Homo sapiens | 39 . . . 962 | 924/924 (100%) | 0.0 |
| | (Human), 924 aa. | 1 . . . 924 | 924/924 (100%) | |
| Q8BIV2 | PROTOCADHERIN 13 homolog - | 13 . . . 962 | 877/951 (92%) | 0.0 |
| | Mus musculus (Mouse), 952 aa. | 2 . . . 952 | 913/951 (95%) | |
| Q8BIZ0 | PROTOCADHERIN 13 homolog - | 39 . . . 962 | 861/925 (93%) | 0.0 |
| | Mus musculus (Mouse), 925 aa. | 1 . . . 925 | 897/925 (96%) | |

PFam analysis predicts that the NOV47a protein contains the domains shown in the Table 47F.

TABLE 47F
Domain Analysis of NOV47a

| | | Identities/ | |
|---|---|---|---|
| | | Similarities | |
| Pfam | NOV47a | for the | Expect |
| Domain | Match Region | Matched Region | Value |
| cadherin | 225 . . . 322 | 36/111 (32%) | 1.3e−08 |
| | | 73/111 (66%) | |
| cadherin | 336 . . . 428 | 27/110 (25%) | 0.0004 |
| | | 66/110 (60%) | |
| cadherin | 551 . . . 641 | 33/107 (31%) | 6.4e−19 |
| | | 69/107 (64%) | |
| cadherin | 655 . . . 744 | 39/107 (36%) | 5.8e−20 |
| | | 69/107 (64%) | |
| cadherin | 762 . . . 855 | 38/112 (34%) | 4e−11 |
| | | 70/112 (62%) | |

### Example 48

The NOV48 clone was analyzed, and the nucleotide and encoded polypeptide sequences are shown in Table 48A.

TABLE 48A
NOV48 Sequence Analysis

| | | |
|---|---|---|
| NOV48a, CG57574-03 | SEQ ID NO: 1053 | 1012 bp |
| DNA Sequence | ORF Start: ATG at 23 | ORF Stop: TAG at 1010 |

AGAGACCAGGTTCTGAGAAGCAATGGTGACGAAGGCCTTTGTCTTGTTGGCCATCTTTGCAGAAGCCT
CTGCAAAATCGTGTGCTCCAAATAAAGCAGATGTCATTCTTGTGTTTTGCTATCCCAAAACCATCATC
ACCAAAATCCCCGAGTGTCCCTATGGATGGGAAGTTCATCAGCTGGCCCTCGGAGGGCTGTGTTACAA
TGGGGTCCACGAAGGAGGTTACTACCAATTTGTGATCCCAGATTTATCACCTAAAAACAAGTCCTATT
GTGGAACCCAGTCTGAGTACAAGCCACCTATCTATCACTTCTACAGTCACATCGTTTCCAATGACACC
ACAGTGATTGTAAAAAACCAGCCTGTCAACTACTCCTTCTCCTGCACCTACCACTCCACCTACTTGGT
GAACCAGGCTGCCTTTGACCAGAGAGTGGCCACTGTTCACGTGAAGAACGGGAGCATGGGCACATTTG
AGAGCCAACTGTCTCTCAACTTCTACACTAATGCCAAGTTCTCCATCAAGAAAGAAGCTCCCTTTGTC
CTGGAGGCATCGGAAATCGGTTCAGATCTGTTTGCAGGAGTGGAAGCCAAAGGGTTAAGCATTAGGTT
TAAAGTGGTCTTGAACAGCTGTTGGGCCACCCCCTCGGCTGACTTCATGTATCCCTTGCAGTGGCAGC
TGATCAACAAGGGCTGCCCCACGGATGAAACCGTCCTCGTGCATGAGAATGGGAGAGATCACAGGGCA
ACCTTCCAATTCAATGCTTTCCGGTTCCAGAACATCCCCAAACTCTCCAAGGTGTGGTTACACTGTGA
GACGTTCATCTGCGACAGTGAGAAACTCTCCTGCCCAGTGACCTGCGATAAACGGAAGCGCCTCCTGC
GAGACCAGACCGGGGGAGTCCTGGTCGTGGAGCTCTCCCTGCGGAGCAGGGGATTTTCCAGTCTCTAT
AGCTTCTCAGATGTTCTCCACCACCTCATCATGATGTTGGGGATTTGTGCCGTGTTATAG

| | | | |
|---|---|---|---|
| NOV48a, CG57574-03 | SEQ ID NO: 1054 | 329 aa | MW at 36955.2kD |
| Protein Sequence | | | |

MVTKAFVLLAIFAEASAKSCAPNKADVILVFCYPKTIITKIPECPYGWEVHQLALGGLCYNGVHEGGY
YQFVIPDLSPKNKSYCGTQSEYKPPIYHFYSHIVSNDTTVIVKNQPVNYSFSCTYHSTYLVNQAAFDQ
RVATVHVKNGSMGTFESQLSLNFYTNAKFSIKKEAPFVLEASEIGSDLFAGVEAKGLSIRFKVVLNSC
WATPSADFMYPLQWQLINKGCPTDETVLVHENGRDHRATFQFNAFRFQNIPKLSKVWLHCETFICDSE
KLSCPVTCDKRKRLLRDQTGGVLVVELSLRSRGFSSLYSFSDVLHHLIMMLGICAVL

| | | |
|---|---|---|
| NOV48b, CG57574-01 | SEQ ID NO: 1055 | 1026 bp |
| DNA Sequence | ORF Start: ATG at 85 | ORF Stop: TAG at 985 |

GATCGAGGCTCAGGCCCTGGAAGGACCGTAAACATTTGGCCAGCTTGGTTTGGATACCTGGCAGAGAC
CAGGTTCTGAGAAGCAATGGTGACGAAGGCCTTTGTCTTGTTGGCCATCTTTGCAGAAGCCTCTGCAA
AATCGTGTGCTCCAAATAAAGCAGATGTCATTCTTGTGTTTTGCTATCCCAAAACCATCATCACCAAA
ATCCCCGAGTGTCCCTATGGATGGGAAGTTCATCAGCTGGCCCTCGGAGGGCTGTGTTACAATGGGGT
CCACGAAGGAGGTTACTACCAATTTGTGATCCCAGATTTATCACCTAAAAACAAGTCCTATTGTGGAA
CCCAGTCTGAGTACAAGCCACCTATCTATCACTTCTACAGTCACATCGTTTCCAATGACACCACAGTG
ATTGTAAAAAACCAGCCTGTCAACTACTCCTTCTCCTGCACCTACCACTCCACCTACTTGGTGAACCA
GGCTGCCTTTGACCAGAGTGTCAATTTCCTTCCAAAGAATGCCAAGTTCTCCATCAAGAAAGAAGCTC
CCTTTGTCCTGGAGGCATCCGAAATCGGTTCAGATCTGTTTGCAGGAGTGGAAGCCAAAGGGTTAAGC
ATTAGGTTTAAAGTGGTCTTGAACAGCTGTTGGGCCACCCCCTCGGCTGACTTCATGTATCCCTTGCA
GTGGCAGCTGATCAACAAGGGCTGCCCCACGGATGAAACCGTCCTCGTGCATGAGAATGGGAGAGATC
ACAGGGCAACCTTCCAATTCAATGCTTTCCGGTTCCAGAACATCCCCAAACTCTCCAAGGTGTGGTTA
CACTGTGAGACGTTCATCTGCGACAGTGAGAAACTCTCCTGCCCAGTGACCTGCGATAAACGGAAGCG
CCTCCTGCGAGACCAGACCGGGGGAGTCCTGGTCGTGGAGCTCTCCCTGCGGAATGTTCTCCACCACC
TCATCATGATGTTGGGGATTTGTGCCGTGTTATAGGAGTTAGCCAGGCAGCTGCCGCTCCTCCACCCA
CAATAG

| | | | |
|---|---|---|---|
| NOV48b, CG57574-01 | SEQ ID NO: 1056 | 300 aa | MW at 33751.8kD |
| Protein Sequence | | | |

MVTKAFVLLAIFAEASAKSCAPNKADVILVFCYPKTIITKIPECPYGWEVHQLALGGLCYNGVHEGGY
YQFVIPDLSPKNKSYCGTQSEYKPPIYHFYSHIVSNDTTVIVKNQPVNYSFSCTYHSTYLVNQAAFDQ
SVNFLPKNAKFSIKKEAPFVLEASEIGSDLFAGVEAKGLSIRFKVVLNSCWATPSADFMYPLQWQLIN
KGCPTDETVLVHENGRDHEATFQFNAFRFQNIPKLSKVWLHCETFICDSEKLSCPVTCDKRKRLLRDQ
TGGVLVVELSLRNVLHHLIMMLGICAVL

| | | |
|---|---|---|
| NOV48c, CG57574-02 | SEQ ID NO: 1057 | 1012 bp |
| DNA Sequence | ORF Start: ATG at 23 | ORF Stop: TAG at 1010 |

AGAGACCAGGTTCTGAGAAGCAATGGTGACGAAGGCCTTTGTCTTGTTGGCCATCTTTGCAGAAGCCT
CTGCAAAATCGTGTGCTCCAAATAAAGCAGATGTCATTCTTGTGTTTTGCTATCCCAAAACCATCATC
ACCAAAATCCCCGAGTGTCCCTATGGATGGGAAGTTCATCAGCTGGCCCTCGGAGGGCTGTGTTACAA
TGGGGTCCACGAAGGAGGTTACTACCAATTTGTGATCCCAGATTTATCACCTAAAAACAAGTCCTATT
GTGGAACCCAGTCTGAGTACAAGCCACCTATCTATCACTTCTACAGTCACATCGTTTCCAATGACGCC
ACAGTGATTGTAAAAAACCAGCCTGTCAACTACTCCTTCTCCTGCACCTACCACTCCACCTACTTGGT
GAACCAGGCTGCCTTTGACCAGAGAGTGGCCACTGTTCACGTGAAGAACGGGAGCATGGGCACATTTG
AGAGCCAACTGTCTCTCAACTTCTACACTAATGCCAAGTTCTCCATCAAGAAAGAAGCTCCCTTTGTC
CTGGAGGCATCCGAAATCGGTTCAGATCTGTTTGCAGGAGTGGAAGCCAAAGGGTTAAGCATTAGGTT
TAAAGTGGTCTTGAACAGCTGTTGGGCCACCCCCTCGGCTGACTTCATGTATCCCTTGCAGTGGCAGC
TGATCAACAAGGGCTGCCCCACGGATGAAACCGTCCTCGTGCATGAGAATGGGAGAGATCACAGGGCA
ACCTTCCAATTCAATGCTTTCCGGTTCCAGAACATCCCCAAACTCTCCAAGGTGTGGTTACACTGTGA
GACGTTCATCTGCGACAGTGAGAAACTCTCCTGCCCAGTGACCTGCGATAAACGGAAGCGCCTCCTGC
GAGACCAGACCGGGGGAGTCCTGGTCGTGGAGCTCTCCCTGCGGAGCAGGGGATTTTCCAGTCTCTAT
AGCTTCTCAGATGTTCTCCACCACCTCATCATGATGTTGGGGATTTGTGCCGTGTTATAG

| | | | |
|---|---|---|---|
| NOV48c, CG57574-02 | SEQ ID NO: 1058 | 329 aa | MW at 36925.2kD |
| Protein Sequence | | | |

MVTKAFVLLAIFAEASAKSCAPNKADVILVFCYPKTIITKIPECPYGWEVHQLALGGLCYNGVHEGGY
YQFVIPDLSPKNKSYCGTQSEYKPPIYHFYSHIVSNDATVIVKNQPVNYSFSCTYHSTYLVNQAAFDQ
RVATVHVKNGSMGTFESQLSLNFYTNAKFSIKKEAPFVLEASEIGSDLFAGVEAKGLSIRFKVVLNSC
WATPSADFMYPLQWQLINKGCPTDETVLVHENGRDHRATFQFNAFRFQNIPKLSKVWLHCETFICDSE
KLSCPVTCDKRKRLLRDQTGGVLVVELSLRSRGFSSLYSFSDVLHHLIMMLGICAVL

| | | |
|---|---|---|
| NOV48d, CG57574-04 | SEQ ID NO: 1059 | 840 bp |
| DNA Sequence | ORF Start: at 7 | ORF Stop: at 835 |

GGATCCAAATCGTGTGCTCCAAATAAAGCAGATGTCATTCTTGTGTTTTGCTATCCCAAAACCATCAT
CACCAAAATCCCCGAGTGTCCCTATGGATGGGAAGTTCATCAGCTGGCCCTCGGAGGGCTGTGTTACA
ATGGGGTCCACGAAGGAGGTTACTACCAATTTGTGATCCCAGATTTATCACCTAAAAACAAGTCCTAT
TGTGGAACCCAGTCTGAGTACAAGCCACCTATCTATCACTTCTACAGTCACATCGTTTCCAATGACAC
CACAGTGATTGTAAAAAACCAGCCTGTCAACTACTCCTTCTCCTGCACCTACCACTCCACCTACTTGG
TGAACCAGGCTGCCTTTGACCAGAGAGTGGCCACTGTTCACGTGAAGAACGGGAGCATGGGCACATTT
GAGAGCCAACTGTCTCTCAACTTCTACACTAATGCCAAGTTCTCCATCAAGAAAGAAGCTCCCTTTGT
CCTGGAGGCATCGGAAATCGGTTCAGATCTGTTTGCAGGAGTGGAAGCCAAAGGGTTAAGCATTAGGT
TTAAAGTGGTCTTGAACAGCTGTTGGGCCACCCCCTCGGCTGACTTCATGTATCCCTTGCAGTGGCAG
CTGATCAACAAGGGCTGCCCCACGGATGAAACCGTCCTCGTGCATGAGAATGGGAGAGATCACAGGGC
AACCTTCCAATTCAATGCTTTCCGGTTCCAGAACATCCCCAAACTCTCCAAGGTGTGGTTACACTGTG
AGACGTTCATCTGCGACAGTGAGAAACTCTCCTGCCCAGTGACCTGCGATAAACGGAAGCGCCTCCTG
CGAGACCAGACCGGGGGACTCGAG

| | | | |
|---|---|---|---|
| NOV48d, CG57574-04 | SEQ ID NO: 1060 | 276 aa | MW at 31202.3kD |
| Protein Sequence | | | |

KSCAPNKADVILVFCYPKTIITKIPECPYGWEVHQLALGGLCYNGVHEGGYYQFVIPDLSPKNKSYCG
TQSEYKPPIYHFYSHIVSNDTTVIVKNQPVNYSFSCTYHSTYLVNQAAFDQRVATVHVKNGSMGTFES
QLSLNFYTNAKFSIKKEAPFVLEASEIGSDLFAGVEAKGLSIRFKVVLNSCWATPSADFMYPLQWQLT
NKGCPTDETVLVHENGRDHPATFQFNAFRFQNIPKLSKVWLHCETFICDSEKLSCPVTCDKRKRLLRD
QTGG

| | | |
|---|---|---|
| NOV48e, CG57574-05 | SEQ ID NO: 1061 | 821 bp |
| DNA Sequence | ORF Start: at 1 | ORF Stop: at 820 |

GGATCCAAATCGTGCGCTCCAAATAAAGCAGATGTCATTCTTGTGTTTTGCTATCCCAAAACCATCAT
CACCAAAATCCCCGAGTGTCCCTATGGATGGGAAGTTCATCAGCTGGCCCTCGGAGGGCTGTGTTACA
ATGGGGTCCACGAAGGAGGTTACTACCAATTTGTGATCCCAGATTTATCACCTAAAAACAAGTCCTAT
TGTGGAACCCAGTCTGAGTACAAGCCACCTATCTATCACTTCTACAGTCACATCGTTTCCAATGACAC
CACAGTGATTGTAAAAAACCAGCCTGTCAACTACTCCTTCTCCTGCACCTACCACTCCACCTACTTGG
TGAACCAGGCTGCCTTTGACCAGAGAGTGGCCACTGTTCACGTGAAGAACGGGAGCATGGGCACATTT
GAGAGCCAACTGTCTCTCAACTTCTACACTAATGCCAAGTTCTCCATCAAGAAAGAAGCTCCCTTTGT
CCTGGAGGCATCCGAAATCGGTTCAGATCTGTTTGCAGGAGTGGAAGCCAAAGGGTTAAGCATTAGGT
TTAAAGTGGTCTTGAACAGCTGTTGGGCCACCCCCTCGGCTGACTTCATGTATCCCTTGCAGTGGCAG
CTGATCAACAAGGGCTGCCCCACGGATGAAACCGTCCTCGTGCATGAGAATGGGAGAGATCACAGGGC
AACCTTCCAATTCAATGCTTTCCGGTTCCAGAACATCCCCAAACTCTCCAAGGTGTGGTTACACTGTG
AGACGTTCATCTGCGACAGTGAGAAACTCTCCTGCCCAGTGACCTGCGATAAACGGAAGCGCCTCCTG
CGAGA

| | | | |
|---|---|---|---|
| NOV48e, CG57574-05 | SEQ ID NO: 1062 | 273 aa | MW at 30888.0kD |
| Protein Sequence | | | |

GSKSCAPNKADVILVFCYPKTIITKIPECPYGWEVHQLALGGLCYNGVHEGGYYQFVIPDLSPKNKSY
CGTQSEYKPPIYHFYSHIVSNDTTVIVKNQPVNYSFSCTYHSTYLVNQAAFDQRVATVHVKNGSMGTF
ESQLSLNFYTNAKFSIKKEAPFVLEASEIGSDLFAGVEAKGLSIRFKVVLNSCWATPSADFMYPLQWQ
LINKGCPTDETVLVHENGRDHRATFQFNAFRFQNIPKLSKVWLHCETFICDSEKLSCPVTCDKRKRLL
R

A ClustalW comparison of the above protein sequences yields the following sequence alignment shown in Table 48B.

TABLE 48B
Comparison of the NOV48 protein sequences.

| | |
|---|---|
| NOV48a | MVTKAFVLLAIFAEASAKSCAPNKADVILVFCYPKTIITKIPECPYGWEVHQLALGGLCY |
| NOV48b | MVTKAFVLLAIFAEASAKSCAPNKADVILVFCYPKTIITKIPECPYGWEVHQLALGGLCY |
| NOV48c | MVTKAFVLLAIFAEASAKSCAPNKADVILVFCYPKTIITKIPECPYGWEVHQLALGGLCY |
| NOV48d | -----------------KSCAPNKADVILVFCYPKTIITKIPECPYGWEVHQLALGGLCY |
| NOV48e | ---------------GSKSCAPNKADVILVFCYPKTIITKIPECPYGWEVHQLALGGLCY |
| NOV48a | NGVHEGGYYQFVIPDLSPKNKSYCGTQSEYKPPIYHFYSHIVSNDTTVIVKNQPVNYSFS |
| NOV48b | NGVHEGGYYQFVIPDLSPKNKSYCGTQSEYKPPIYHFYSHIVSNDTTVIVKNQPVNYSFS |
| NOV48c | NGVHEGGYYQFVIPDLSPKNKSYCGTQSEYKPPIYHFYSHIVSNDATVIVKNQPVNYSFS |
| NOV48d | NGVHEGGYYQFVIPDLSPKNKSYCGTQSEYKPPIYHFYSHIVSNDTTVIVKNQPVNYSFS |
| NOV48e | NGVHEGGYYQFVIPDLSPKNKSYCGTQSEYKPPIYHFYSHIVSNDTTVIVKNQPVNYSFS |
| NOV48a | CTYHSTYLVNQAAFDQRVATVHVKNGSMGTFESQLSLNFYTNAKFSIKKEAPFVLEASEI |
| NOV48b | CTYHSTYLVNQAAFDQSVNFLPK-----------------N-AKFSIKKEAPFVLEASEI |
| NOV48c | CTYHSTYLVNQAAFDQRVATVHVKNGSMGTFESQLSLNFYTNAKFSIKKEAPFVLEASEI |
| NOV48d | CTYHSTYLVNQAAFDQRVATVHVKNGSMGTFESQLSLNFYTNAKFSIKKEAPFVLEASEI |
| NOV48e | CTYHSTYLVNQAAFDQRVATVHVKNGSMGTFESQLSLNFYTNAKFSIKKEAPFVLEASEI |
| NOV48a | GSDLFAGVEAKGLSIRFKVVLNSCWATPSADFMYPLQWQLINKGCPTDETVLVHENGRDH |
| NOV48b | GSDLFAGVEAKGLSIRFKVVLNSCWATPSADFMYPLQWQLINKGCPTDETVLVHENGRDH |
| NOV48c | GSDLFAGVEAKGLSIRFKVVLNSCWATPSADFMYPLQWQLINKGCPTDETVLVHENGRDH |
| NOV48d | GSDLFAGVEAKGLSIRFKVVLNSCWATPSADFMYPLQWQLINKGCPTDETVLVHENGRDH |
| NOV48e | GSDLFAGVEAKGLSIRFKVVLNSCWATPSADFMYPLQWQLINKGCPTDETVLVHENGRDH |
| NOV48a | RATFQFNAFRFQNIPKLSKVWLHCETFICDSEKLSCPVTCDKRKRLLRDQTGGVLVVELS |
| NOV48b | RATFQFNAFRFQNIPKLSKVWLHCETFICDSEKLSCPVTCDKRKRLLRDQTGGVLVVELS |
| NOV48c | RATFQFNAFRFQNIPKLSKVWLHCETFICDSEKLSCPVTCDKRKRLLRDQTGGVLVVELS |
| NOV48d | RATFQFNAFRFQNIPKLSKVWLHCETFICDSEKLSCPVTCDKRKRLLRDQTGG------- |
| NOV48e | RATFQFNAFRFQNIPKLSKVWLHCETFICDSEKLSCPVTCDKRKRLLR------------ |
| NOV48a | LRSRGFSSLYSFSDVLHHLIMMLGICAVL |
| NOV48b | LRNVLHHLIMMLGICAVL----------- |
| NOV48c | LRSRGFSSLYSFSDVLHHLIMMLGICAVL |
| NOV48d | ----------------------------- |
| NOV48e | ----------------------------- |
| NOV48a | (SEQ ID NO: 1054) |
| NOV48b | (SEQ ID NO: 1056) |
| NOV48c | (SEQ ID NO: 105B) |
| NOV48d | (SEQ ID NO: 1060) |
| NOV48e | (SEQ ID NO: 1062) |

Further analysis of the NOV48a protein yielded the following properties shown in Table 48C.

TABLE 48C
Protein Sequence Properties NOV48a

| | |
|---|---|
| SignalP analysis: | Cleavage site between residues 18 and 19 |
| PSORT II analysis: | |

| PSG: | a new signal peptide prediction method | |
|---|---|---|
| | N-region: | length 4; pos. chg 1; neg. chg 0 |
| | H-region: | length 9; peak value 11.46 |
| | PSG score: | 7.06 |

| | |
|---|---|
| GvH: | von Heijne's method for signal seq. recognition |
| | GvH score (threshold: −2.1): 0.86 |
| | possible cleavage site: between 17 and 18 |

>>> Seems to have a cleavable signal peptide (1 to 17)

| ALOM: | Klein et al's method for TM region allocation | |
|---|---|---|
| | Init position for calculation: 18 | |
| | Tentative number of TMS(s) for the threshold 0.5: 1 | |
| | Number of TMS(s) for threshold 0.5: 0 | |
| | PERIPHERAL | Likelihood = 2.76 (at 25) |

| | ALOM score: −1.44 (number of TMSs: 0) | | | |
|---|---|---|---|---|
| MTOP: | Prediction of membrane topology (Hartmann et al.) | | | |
| | Center position for calculation: 8 | | | |
| | Charge difference: −2.0 C(0.0)-N(2.0) | | | |
| | N >= C: N-terminal side will be inside | | | |
| MITDISC: | discrimination of mitochondrial targeting seq | | | |
| | R content: | 0 | Hyd Moment(75): | 8.58 |
| | Hyd Moment(95): | 5.88 | G content: | 0 |
| | D/E content: | 2 | S/T content: | 3 |

| | |
|---|---|
| | Score: −5.83 |
| Gavel: | prediction of cleavage sites for mitochondrial preseq |
| | cleavage site motif not found |

NUCDISC: discrimination of nuclear localization signals

| | |
|---|---|
| | pat4: KRKR (5) at 282 |
| | pat7: none |
| | bipartite: none |
| | content of basic residues: 9.4% |
| | NLS Score: −0.16 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: CaaX motif in the C-terminus: CAVL
if X is S, A, or M, it will be farnesylated
otherwise, it will be geranylgeranylated
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear |
| | discrimination |
| | Prediction: cytoplasmic |
| | Reliability: 94.1 |
| COIL: | Lupas's algorithm to detect coiled-coil regions |
| | total: 0 residues |

Final Results (k = 9/23):

33.3%: endoplasmic reticulum
22.2%: mitochondrial
11.1%: cytoplasmic
11.1%: vacuolar
11.1%: nuclear
11.1%: extracellular, including cell wall

>> prediction for CG57574-03 is end (k = 9)

A search of the NOV48a protein against the Geneseq database, a proprietary database that contains sequences published in patents and patent publication, yielded several homologous proteins shown in Table 48D.

TABLE 48D
Geneseq Results for NOV48a

| | | NOV48a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE17314 | Human tectorin beta protein, | 1 . . . 329 | 329/329 (100%) | 0.0 |
| | sbg453915TECTORINa - Homo | 1 . . . 329 | 329/329 (100%) | |
| | sapiens, 329 aa. [WO200198342-A1, | | | |
| | 27-DEC-2001] | | | |
| ABG72418 | Cytochrome b45.65 - Unidentified, 415 | 14 . . . 285 | 84/299 (28%) | 3e−17 |
| | aa. [CN1345764-A, 24-APR-2002] | 27 . . . 318 | 131/299 (43%) | |
| AAE17559 | Human pancreatic tumour protein, GP- | 77 . . . 282 | 62/215 (28%) | 1e−12 |
| | 2 - Homo sapiens, 530 aa. | 270 . . . 472 | 101/215 (46%) | |
| | [WO200194409-A2, 13-DEC-2001] | | | |
| AAB54274 | Human pancreatic cancer antigen | 77 . . . 282 | 62/215 (28%) | 1e−12 |
| | protein sequence SEQ ID NO: 726 - | 146 . . . 348 | 101/215 (46%) | |
| | Homo sapiens, 406 aa. | | | |
| | [WO200055320-A1, 21-SEP-2000] | | | |
| AAR98963 | Human GP2 pancreatic glycoprotein - | 77 . . . 282 | 62/215 (28%) | 1e−12 |
| | Homo sapiens, 384 aa. [WO9617873- | 124 . . . 326 | 101/215 (46%) | |
| | A1, 13-JUN-1996] | | | |

In a BLAST search of public sequence databases, the NOV48a protein was found to have homology to the proteins shown in the BLASTP data in Table 48E.

TABLE 48E
Public BLASTP Results for NOV48a

| | | NOV48a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| Q96PL2 | Tectorin beta - Homo sapiens | 1 . . . 329 | 329/329 | (100%) | 0.0 |
| | (Human), 329 aa. | 1 . . . 329 | 329/329 | (100%) | |
| O08524 | Beta tectorin - Mus musculus | 1 . . . 329 | 310/329 | (94%) | 0.0 |
| | (Mouse), 329 aa. | 1 . . . 329 | 317/329 | (96%) | |
| P54097 | Beta-tectorin precursor - Gallus | 1 . . . 329 | 242/329 | (73%) | e−150 |
| | gallus (Chicken), 329 aa. | 1 . . . 328 | 286/329 | (86%) | |
| Q95JJ6 | Hypothetical 45.5 kDa protein - | 14 . . . 285 | 85/299 | (28%) | 4e−17 |
| | Macaca fascicularis (Crab eating | 27 . . . 318 | 131/299 | (43%) | |
| | macaque) (Cynomolgus monkey), | | | | |
| | 415 aa. | | | | |
| Q8TCW7 | Hypothetical protein - Homo sapiens | 14 . . . 285 | 84/299 | (28%) | 5e−17 |
| | (Human), 431 aa. | 43 . . . 334 | 131/299 | (43%) | |

PFam analysis predicts that the NOV48a protein contains the domains shown in the Table 48F.

TABLE 48F
Domain Analysis of NOV48a

| | | | |
|---|---|---|---|
| | | Identities/ | |
| | | Similarities | |
| Pfam | NOV48a | for the | Expect |
| Domain | Match Region | Matched Region | Value |

| | | | | |
|---|---|---|---|---|
| zona_pellucida | 19 . . . 283 | 55/310 | (18%) | 8.2e−49 |
| | | 190/310 | (61%) | |

### Example 49

The NOV49 clone was analyzed, and the nucleotide and encoded polypeptide sequences are shown in Table 49A.

TABLE 49A
NOV49 Sequence Analysis

| | | |
|---|---|---|
| NOV49a, CG57689-01 | SEQ ID NO: 1063 | 1933 bp |
| DNA Sequence | ORF Start: ATG at 75 | ORF Stop: TAA at 1881 |

GGTCGGCTGCCCCGAGGGCTTTGAGCTGGACTCCCAGGGAGCGTTTTGTGTGGGTGAGCGCCCCCAAC
CCTGGCATGGATGTGGGAGGCCTCTCAGCCTGGGATGGATGGCTTCTCCCAGCCAGCTGGCAGGAGGG
CCTGGGAGCGGAGACCACCAGACCCCTTCCTGCCCCAGACAGGGACGAGTGCTCAGGAGGCCCTAGCC
CCTGCTCCCATGCCTGCCTTAATGCACCCGGCCGCTTCTCCTGCACCTGCCCCACTGGCTTCGCCCTG
GCCTGGGATGACAGGAACTGCAGAGATGTGGACGAGTGTGCGTGGGATGCTCACCTCTGCCGAGAGGG
ACAGCGCTGTGTGAACCTGCTCGGGTCCTACCGCTGCCTCCCCGACTGTGGGCCTGGCTTCCGGGTGG
CTGATGGGGCCGGCTGTGAAGATGTGGACGAATGCCTGGAGGGGTTGGACGACTGTCACTACAACCAG
CTCTGCGAGAACACCCCAGGCGGTCACCGCTGCAGCTGCCCCAGGGGTTACCGGATGCAGGGCCCCAG
CCTGCCCTGCCTAGATGTCAATGAGTGCCTGCAGCTGCCCAAGGCCTGCGCCTACCAGTGCCACAACC
TCCAGGGCAGCTACCGCTGCCTGTGCCCCCCAGGCCAGACCCTCCTTCGCGACGGCAAGGCCTGCACC
TCACTGGAGCGGAATGGACAAAATGTGACCACCGTCAGCCACCGAGGCCCTCTATTGCCCTGGCTGCG
GCCCTGGGCCTCGATCCCCGGTACCTCCTACCACGCCTGGGTCTCTCTCCGTCCGGGTCCCATGGCCC
TGAGCAGTGTGGGCCGGGCCTGGTGCCCTCCTGGTTTCATCAGGCAGAACGGAGTCTGCACAGACCTT
GACGAGTGCCGCGTGAGGAACCTGTGTCAGCACGCCTGCCGCAACACTGAGGGCAGCTACCAGTGCCT
GTGCCCACGCCGGCTACCGTCTGCTCCCCAGCGGGAAGAACTGCCAGGACATCAACGAGTGCGAGGAG
GAGAGCATCGAGTGTGGACCCGGCCAGATGTGCTTCAACACCCGTGGCAGCTACCAGTGTGTGGACAC
ACCCTGTCCTGCCACCTACCGGCAGGGCCCCAGCCCTGGGACGTGCTTCCGGCGCTGCTCGCAGGACT
GCGGCACGGGCGGCCCCTCTACGCTGCAGTACCGGCTGCTGCCGCTGCCCCTGGGCGTGCGCGCCCAC
CACGACGTGGCCCGCCTCACCGCCTTCTCCGAGGTCGGCGTCCCCGCCAACCGCACCGAGCTCAGCAT
GCTGGAGCCCGACCCCCGCAGCCCCTTCGCGCTGCGTCCGCTGCGCGCGGGCCTTGGCGCGGTCTACA
CCCGTCGCGCGCTCACCCGCGCCGGCCTCTACCGGCTCACCGTGCGTGCTGCGGCACCGCGCCACCAA
AGCGTCTTCGTCTTGCTCATCGCCGTGTCCCCCTACCCCTACTAAACGGGAGAGGGCATTGGCGGCCG
CCCTGGCGTGACCCCCGAGGAAGGGGTCGAGGAGAAGCTTGGTCCACGCCACCTGCTGTGGCAAGCGG
AGCGTCATCGTCTCCCGCCCCGTGCGTCAGCGAGACCTTGGGTCAACACGACCCTGCGCACAGCCTTG
ACCCCCGACAGCGAGGACCTGACCTCACAGAGGGAGGCGTCCAGGGCGGCCCTTGGGTGGCCAGTCCC
GCAGGCAGGGCCCGGGGAAGCCCGGATCAGACCTCCAGGTCTGATCCGCCCCTCAGTGGGAGCGGGAC
AGGGACACAGGGCACCTGGACGCGCGGGAGAGGGGGCAGACCCCGCGTTAGGGGTGGCAGCAGCTGTC
GCCCGGCCACACCTGGTGGTGTCATTCTGAACCCTGTTGCAATATAAAGGGATTTTTTTTTAACCAAC
TTGGTTTCTTTGTCTAATTATTTTGCTTT

| | | | |
|---|---|---|---|
| NOV49a, CG57689-01 | SEQ ID NO: 1064 | 602 aa | MW at 65011.8kD |
| Protein Sequence | | | |

MDVGGLSAWDGWLLPASWQEGLGAETTRPLPAPDRDECSGGPSPCSHACLNAPGRFSCTCPTGFALAW
DDRNCRDVDECAWDAHLCREGQRCVNLLGSYRCLPDCGPGFRVADGAGCEDVDECLEGLDDCHYNQLC
ENTPGGHRCSCPRGYRMQGPSLPCLDVNECLQLPKACAYQCHNLQGSYRCLCPPGQTLLRDGKACTSL
ERNGQNVTTVSHRGPLLPWLRPWASIPGTSYHAWVSLRPGPMALSSVGRAWCPPGFIRQNGVCTDLDE
CRVRNLCQHACRNTEGSYQCLCPRRLPSAPQREELPGHQRVRGGEHRVWTRPDVLQHPWQLPVCGHTL
SCHLPAGPQPWDVLPALLAGLRHGRPLYAAVPAAAAAPGRARPPRRGPPHRLLRGRRPRQPHRAQHAG
ARPPQPLRAASAARGPWRGLHPSRAHPRRPLPAHRACCGTAPPKRLRLAHRRVPLPLLNGRGHWRPPW
RDPRGRGRGEAWSTPPAVASGASSSPAPCVSETLGQHDPAHSLDPRQRGPDLTEGGVQGGPWVASPAG
RARGSPDQTSRSDPPLSGSGTGTQGTWTRGRGGRPRVRGGSSCRPATPGGVILNPVAI

Further analysis of the NOV49a protein yielded the following properties shown in Table 49B.

TABLE 49B
Protein Sequence Properties NOV49a

| | |
|---|---|
| SignalP analysis: | No Known Signal Sequence Predicted |
| PSORT II analysis: | |

| PSG: | a new signal peptide prediction method | |
|---|---|---|
| | N-region: | length 10; pos. chg 0; neg. chg 2 |
| | H-region: | length 9; peak value 0.00 |
| | PSG score: | −4.40 |

| | |
|---|---|
| GvH: | von Heijne's method for signal seq. recognition |
| | GvH score (threshold: −2.1): −3.99 |
| | possible cleavage site: between 23 and 24 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 0 | |
| | number of TMS(s) . . . fixed | |
| | PERIPHERAL | Likelihood = 7.80 (at 586) |

| | ALOM score: 7.80 (number of TMSs: 0) | | | |
|---|---|---|---|---|
| MITDISC: | discrimination of mitochondrial targeting seq | | | |
| | R content: | 0 | Hyd Moment(75): | 3.89 |
| | Hyd Moment(95): | 4.99 | G content: | 2 |
| | D/E content: | 2 | S/T content: | 1 |

| | |
|---|---|
| | Score: −8.14 |
| Gavel: | prediction of cleavage sites for mitochondrial preseq |
| | cleavage site motif not found |

NUCDISC: discrimination of nuclear localization signals

| | |
|---|---|
| | pat4: RRPR (4) at 396 |
| | pat7: PPKRLRL (4) at 450 |
| | pat7: PKRLRLA (5) at 451 |
| | bipartite: none |
| | content of basic residues: 12.3% |
| | NLS Score: 0.55 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear |
| | discrimination |
| | Prediction: nuclear |
| | Reliability: 94.1 |
| COIL: | Lupas's algorithm to detect coiled-coil regions |
| | total: 0 residues |

Final Results (k = 9/23):

```
73.9%: nuclear
17.4%: cytoplasmic
  4.3%: mitochondrial
  4.3%: peroxisomal
```

>> prediction for CG57689-01 is nuc (k = 23)

A search of the NOV49a protein against the Geneseq database, a proprietary database that contains sequences published in patents and patent publication, yielded several homologous proteins shown in Table 49C.

TABLE 49C
Geneseq Results for NOV49a

| | | NOV49a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABU07377 | Human protein NOV9 - Homo sapiens, | 34 . . . 307 | 266/277 (96%) | e−172 |
| | 3931 aa. [WO200285922-A2, 31-OCT- | 3500 . . . 3776 | 269/277 (97%) | |
| | 2002] | | | |
| ABP69271 | Human polypeptide SEQ ID NO 1318 - | 34 . . . 307 | 266/277 (96%) | e−172 |
| | Homo sapiens, 939 aa. | 508 . . . 784 | 269/277 (97%) | |
| | [WO200270539-A2, 12-SEP-2002] | | | |
| ABG34007 | Human secreted protein #4 encoded by | 60 . . . 307 | 231/251 (92%) | e−146 |
| | gene 57 - Homo sapiens, 638 aa. | 233 . . . 483 | 234/251 (93%) | |
| | [WO200226931-A2, 04-APR-2002] | | | |
| ABG33919 | Human secreted protein encoded by | 60 . . . 307 | 231/251 (92%) | e−146 |
| | gene 57 #1 - Homo sapiens, 575 aa. | 170 . . . 420 | 234/251 (93%) | |
| | [WO200226931-A2, 04-APR-2002] | | | |
| AAE06183 | Human gene 57 encoded secreted | 60 . . . 307 | 231/251 (92%) | e−146 |
| | protein fragment, SEQ ID NO: 245 - | 233 . . . 483 | 234/251 (93%) | |
| | Homo sapiens, 638 aa. | | | |
| | [WO200151504-A1, 19-JUL-2001] | | | |

In a BLAST search of public sequence databases, the NOV49a protein was found to have homology to the proteins shown in the BLASTP data in Table 49D.

TABLE 49D
Public BLASTP Results for NOV49a

| | | NOV49a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8BNH3 | Inferred: hemicentin - Mus | 34 . . . 546 | 285/541 (52%) | e−148 |
| | musculus (Mouse), 729 aa. | 229 . . . 729 | 314/541 (57%) | |
| Q8K061 | Hypothetical protein - Mus | 34 . . . 307 | 212/278 (76%) | e−135 |
| | musculus (Mouse), 787 aa. | 355 . . . 632 | 229/278 (82%) | |
| Q96SC3 | Fibulin-6 - Homo sapiens | 26 . . . 317 | 124/302 (41%) | 5e−62 |
| | (Human), 2673 aa (fragment). | 2222 . . . 2506 | 159/302 (52%) | |
| Q96RW7 | Hemicentin - Homo sapiens | 26 . . . 317 | 122/302 (40%) | 1e−60 |
| | (Human), 5636 aa. | 5185 . . . 5469 | 158/302 (51%) | |
| Q96K89 | Hypothetical protein FLJ14438 - | 34 . . . 317 | 114/297 (38%) | 4e−48 |
| | Homo sapiens (Human), 741 aa. | 330 . . . 574 | 147/297 (49%) | |

PFam analysis predicts that the NOV49a protein contains the domains shown in the Table 49E.

TABLE 49E
Domain Analysis of NOV49a

| | | Identities/ | |
|---|---|---|---|
| | | Similarities | |
| Pfam | NOV49a | for the | Expect |
| Domain | Match Region | Matched Region | Value |
| EGF | 38 . . . 73 | 14/47 (30%) | 2.9e−07 |
| | | 29/47 (62%) | |
| EGF | 123 . . . 160 | 14/47 (30%) | 0.86 |
| | | 24/47 (51%) | |
| EGF | 166 . . . 201 | 15/47 (32%) | 8.6e−06 |
| | | 26/47 (55%) | |

### Example 50

The NOV50 clone was analyzed, and the nucleotide and encoded polypeptide sequences are shown in Table 50A.

TABLE 50A
NOV50 Sequence Analysis

| | | |
|---|---|---|
| NOV50a, CG58567-01 | SEQ ID NO: 1065 | 3373 bp |
| DNA Sequence | ORF Start: at 1 | ORF Stop: TGA at 3190 |

TTGATTGTCCAAGCCACAGATAAAGGGATGCCCAGGCTTTCTAATACGACTGTAATCAAGGTACAGGT
GACTGATATAAATGACAATGCCCCAGCTTTTCTCCCCTCTGAAGCAGTGGAAATTACAGAAGTCATGA
CTATTTCAGAAGATTCTTTGCCTGGTGTAATTGTGACTCATGTGTCAGTTCATGATGTGGATTTGAAT
TCAGCTTTCATATTCAGTTTTGCCAAAGAGAGTAATCCTGGAACCAAGTTTGCTATTGATCAGAACAC
TGGAGTGGTGGTGTTGGTGAAAACATTGGATTTTGAAGAAATGACTGAATATGAGCTGCTCATCCAAA
TTTCTGATTCAGTGCACTACACAGAGGGAGCACTTGTAGTCCGTGTGCTGGATGTCAATGATAATCCA
CCAGTGTTTTCTCAAGATTTCTATCAGGTCACAGTTCCTGAATCAATACCTGTGGGGTATTCAGTGCT
GACTCTGTCAGCCACAGACTTAGAAAGCAATGAGAACATTTCTTACAGAATTCTATCCTCTTCTAAGG
AATTCTCCATTGATCCTAAGAATGGCACAATATTTACTATCAGTCCCGTATTACTTCTGGATACAATA
TCAACAACTCGATTTCTTGTGGAAGCCAGTGATGGTGGAAATCCTGACCCGAGAGCTCTTACTTTAGT
GGAGATAGGAATAGAAGATATGAACAATTATGCCCCTGAATTCACAGTCAAATCCTATAATCTTAGCC
TAAGTGAGGATGCTCTGGTTGGAAGCACGCTTGTTACATTTTCAAACATCGACCATGACTGGACCCGT
GAAAACACATATGTTGAATATTCCATCATCAGTGGTAATTCACAGAACAATTTTCATGTGGAAACTAA
GTTCTTTCATTCAGAATATCCTTATAAGCAAGTCGGTTATCTTGTGTTGCTTCACAGTCTGGACAGAG
AAGCAAGTGCTAGCCATGAGCTTGTCATTCTGGCATCTGACAGTGGCTGCCCTCCATTGAGTTCCACA
GCTGTCATATCAATACAAGTACTTGATGTCAATGACAATCCCCCAAACTTCAGCAGCCTGAGCTATCA
CACCCATGTCAAGGAAAGCACCCCTCTAGGGAGTCACATCACTGTGGTCTCAGCAAATGACCGTGACA
CAGGGTCACATGCAGAAATCATCTACAACATCATCTCTGGAAATGAGAAGGGACATTTTTACTTAGAA
GAAAACACTGGAGTTCTTTATTTGATTAAACCTCTGGATTATGAAAAAATGACAAAATTCACCTTAAC
TGTCCAAGCTTCAGATGCAGAAAAGAAACATTTTTCTTTTGCAGTTGTGTTTGTCAGTGTCCTGGATG
ATAACGACCATGCACCTCAGTTTATGTTCTCAAGCTTCAGCTGTATTGTTCCAGAAAATCTGCCTATT
TCCTCTACCATATGCTCTATAAATGCTCTGGATTTTGATGCTGGTCCGTATGGAGAATTGACCTATTC
TATTGTATCACCCTGTTTTCTCACTCATGGAATGTCTTATGATCATGATCTCTTCCTCATTGACCCTT
TGACAGGGGATATTCATGCTAAGCAAATCCTTGACTATGAAAATGGCAATAAATACTGCCTCACAGTC
CAAGCCAAAGACAAAGGTGATGCAACTGCCTCCTTAGTGGTCTGGGTGGATATTGAAGGGATAGATGA
ATTTGAGCCCATTTTCACTCAAGATCAGTATTTTTTCACCCTCCCAGAAAAGAATAAAGACAGACAGT
TGATTGGCAGAGTGGAAGCCTCAGATGCAGATGCTGGTATTGATGGAGTCATTCTTTACTCCCTTGGA
ACCTCATCTCCTTTCTTTTCAGTAAATAGAACCAATGGAAATATTTATTTGATTAGAGCCCTTCCCCT
AATAAAAAGTCAACTCAACAAAGAAGACACCTTGGAAATGAAAATAATCGCTCATAGTCCCAAATCAG
ATTCCAAGTTTGCATCTTGCACTGTTTTTGTGAATGTGTCTTTCTCCTCTGAAGGAACACCCTTGGCA
GTGTTCGCCAGCAGCTTTTCAATCAGCCTGGTGGTCTCCTTTTTAGTGTTTCTGATACTCATCTGCAT
TCTAATTGTAATGATTTTAAGACATAAACAAAAAGACACAATAAACAATTATGAGGAGAAGAAAACCT
CATCTTTAGATGCGGACTTGAGAGTGACCCGGGATGCCAGTGTGCTCAAAGCCTTCCAGAAAACTGAC
GACTGCAGTAACGAGGTGGTCCCTGTGGATGCCACTCCGGAATGGTTGAGTTTAATAAGTATCATGGA
GAAGGATATTGTCAATCTGTACAGATACTCAAACTCCAGTGGCCACTGTTCTGTGGAAGGAGAAACTG
CAGAAGATAAGGAAATCCAGAGGATAAATGAGCATCCCTACAGAAAGTGCTCAGACTCAGCTCTGAGT
GACCACGAGTCCAGGGTGCCAGACTCGGGTATCCCGAGGGACTCAGACCAGCTCTCCTGCCTATCTGG
GGAAACTGATGTGATGGTGACTGCCGAAACAGCAGAAGCCAGCCAAACATTTGGGGAAGGAGATCAAG
GGGAAGGCTGCAGCACCACCTGTGCTCAAAATAATGTGTTACCCCAGACAGTTCAGAAGAGAGAGGCA
AAAGAGAGCATCCTGGCTGACGTTAGAAAAGAGTCTGTCTTTATTTCAGGTGATCAGGAAGTAAGGTG
TGCAGCTCTTTCAACTCAGACGACCTCTGATCATGATGGAAAAGACAACTATCACTGGAATTATCTTC
TTAGTTGGGAGCCCAAATTCCAACCTCTTGCCTCAGTATTTAATGATATTGCAAAACTAAAGGATGAA
CATTTGCATATGCCTGGCATTCCAAAAGAGAAGAAATCTTTTGTTTTTCCACCCCCTTTGATAACAGC
AGTAGCCCAGCCTGGGATTAAAGCAGTCCCACCAAGAATGCCGGCAGTAAACCTGGGGCAGGTGCCTC
CGAAACACCCACGCTCTCCCATCCCCTACCATCTTGGTTCTCTGCCAGAAGGCATGACTCCCAATTTT
TCTCCATCTCTTTCCCTATTGACGATGCAGCCTCCTGCCTTGTCTCCACTGTTGAGAGAAGGAGAATT
ATTAGGAACACACATCAGTGGTACATGCCATGAACTTAAAGCAGAAGATGAAGTTCAAATATGAAACC
ACTGGGATGCCAAGTACCTGCTCACCATTGGTCATGAATGAATGAACAAAATGTTTTCAAGCCGGCAA
CTCGAGATTGGGCTCATTTTTATCTAAAAGCAAGTGATGTAATTTAGTTAGAGTTTTTAAAACTTCCC
CATTAAAGTTTCTCCAATTTCAAAAAAAAAAAAAAAAAAAA

| | | | |
|---|---|---|---|
| NOV50a, CG58567-01 | SEQ ID NO: 1066 | 1063 aa | MW at 117200.4kD |
| Protein Sequence | | | |

LIVQATDKGMPRLSNTTVIKVQVTDINDNAPAFLPSEAVEITEVMTISEDSLPGVIVTHVSVHDVDLN
SAFIFSFAKESNPGTKFAIDQNTGVVVLVKTLDFEEMTEYELLIQISDSVHYTEGALVVRVLDVNDNP
PVFSQDFYQVTVPESIPVGYSVLTLSATDLESNENISYRILSSSKEFSIDPKNGTIFTISPVLLLDTI
STTRFLVEASDGGNPDPRALTLVEIGIEDMNNYAPEFTVKSYNLSLSEDALVGSTLVTFSNIDHDWTR
ENTYVEYSIISGNSQNNFHVETKFFHSEYPYKQVGYLVLLHSLDREASASHELVILASDSGCPPLSST
AVISIQVLDVNDNPPNFSSLSYHTHVKESTPLGSHITVVSANDRDTGSHAEIIYNIISGNEKGHFYLE
ENTGVLYLIKPLDYEKMTKFTLTVQASDAEKKHFSFAVVFVSVLDDNDHAPQFMFSSFSCIVPENLPI
SSTICSINALDFDAGPYGELTYSIVSPCFLTHGMSYDHDLFLIDPLTGDIHAKQILDYENGNKYCLTV
QAKDKGDATASLVVWVDIEGIDEFEPIFTQDQYFFTLPEKNKDRQLIGRVEASDADAGIDGVILYSLG
TSSPFFSVNRTNGNIYLIRALPLIKSQLNKEDTLEMKIIAHSPKSDSKFASCTVFVNVSFSSEGTPLA
VFASSFSISLVVSFLVFLILICILIVMILRHKQKDTINNYEEKKTSSLDADLRVTRDASVLKAFQKTD
DCSNEVVPVDATPEWLSLISIMEKDIVNLYRYSNSSGHCSVEGETAEDKEIQRINEHPYRKCSDSALS
DHESRVPDSGIPRDSDQLSCLSGETDVMVTAETAEASQTFGEGDQGEGCSTTCAQNNVLPQTVQKREA
KESILADVRKESVFISGDQEVRCAALSTQTTSDHDGKDNYHWNYLLSWEPKFQPLASVFNDIAKLKDE
HLHMPGIPKEKKSFVFPPPLITAVAQPGIKAVPPRMPAVNLGQVPPKHPRSPIPYHLGSLPEGMTPNF
SPSLSLLTMQPPALSPLLREGELLGTHISGTCHELKAEDEVQI

| | | |
|---|---|---|
| NOV50b, 220087646 | SEQ ID NO: 1067 | 3075 bp |
| DNA Sequence | ORF Start: at 1 | ORF Stop: end of sequence |

GGATCCGAAGTCATGACTATTTCAGAAGATTCTTTGCCTGGTGTAATTGTGACTCATGTGTCAGTTCA
TGATGTGGATTTGAATTCAGCTTTCATATTCAGTTTTGCCAAAGAGAGTAATCCTGGAACCAAGTTTG
CTATTGATCAGAACACTGGAGTGGTGGTGTTGGTGAAAACATTGGATTTTGAAGAAATGACTGAATAT
GAGCTGCTCATCCAAATTTCTGATTCAGTGCACTACACAGAGGGAGCACTTGTAGTCCGTGTGCTGGA
TGTCAATGATAATCCACCAGTGTTTTCTCAAGATTTCTATCAGGTCACAGTTCCTGAATCAATACCTG
TGGGGTATTCAGTGCTGACTCTGTCAGCCACAGACTTAGAAAGCAATGAGAACATTTCTTACAGAATT
CTATCCTCTTCTAAGGAATTCTCCATTGATCCTAAGAATGGCACAATATTTACTATCAGTCCCGTATT
ACTTCTGGATACAATATCAACAACTCAATTTCTTGTGGAAGCCAGTGATGGTGGAAATCCTGACCTGA
GAGCTCTTACTTTAGTGGAGATAGGAATAGAAGATATGAACAATTATGCCCCTGAATTCACAGTCAAA
TCCTATAATCTTAGCCTAAGTGAGGATGCTCTGGTTGGAAGCACGCTTGTTACATTTTCAAACATCGA
CCATGACTGGACCCGTGAAAACACATATGTTGAATATTCCATCATCAGTGGTAATTCACAGAACAATT
TTCATGTGGAAACTAAGTTCTTTCATTCAGAATATCCTTATAAGCAGGTCGGTTATCTTGTGTTGCTT
CACAGTCTGGACAGAGAAGCAAGTGCTAGCCATGAGCTTGTCATTCTGGCATCTGACAGTGGCTGCCC
TCCATTGAGTTCCACAGCTGTCATATCAATACAAGTACTTGATGTCAATGACAATCCCCCAAACTTCA
GCAGCCTGAGCTATCACACCCATGTCAAGGAAAGCACCCCTCTAGGGAGTCACATCACTGTGGTCTCA
GCAAATGACCGTGACACAGGGTCACATGCAGAAATCATCTACAACATCATCTCTGGAAATGAGAAGGG
ACATTTTTACTTAGAAGAAAACACTGGAGTTCTTTATTTGATTAAACCTCTGGATTATGAAAAAATGA
CAAAATTCACCTTAACTGTCCAAGCTTCAGATGCAGAAAAGAAACATTTTTCTTTTGCAGTTGTGTTT
GTCAGTGTCCTGGATGATAACGACCATGCACCTCAGTTTATGTTCTCAAGCTTCAGCTGTATTGTTCC
AGAAAATCTGCCTATTTCCTCTACCATACGCTCTATAAATGCTCTGGATTTTGATGCTGGTCCGTATG
GAGAATTGACCTATTCTATTGTATCACCCTGTTTTCTCACTCATGGAATGTCTTATGATCATGATCTC
TTCCTCATTGACCCTTTGACAGGGGATATTCATGCTAAGCAAATCCTTGACTATGAAAATGGCAATAA
ATACTGCCTCACAGTCCAAGCCAAAGACAAAGGTGATGCAACTGCCTCCTTAGTGGTCTGGGTGGATA
TTGAAGGGATAGATGAATTTGAGCCCATTTTCACTCAAGATCAGTATTTTTTCACCCTCCCAGAAAAG
AATAAAGACAGACAGTTGATTGGCAGAGTGGAAGCCTCAGATGCAGATGCTGGTATTGATGGAGTCAT
TCTTTACTCCCTTGGAACCTCATCTCCTTTCTTTTCAGTAAATAAAACCAATGGAAATATTTATTTGA
TTAGAGCCCTTCCCCTAATAAAAAGTCAACTCAACAAAGAAGACACCTTGGAAATGAAAATAATCGCT
CATAGTCCCAAATCAGATTCCAAGTTTGCATCTTGCACTGTTTTTGTGAATGTGTCTTTCTCCTCTGA
AGGAACACCCTTGGCAGTGTTCGCCAGCAGCTTTTCAATCAGCCTGGTGGTCTCCTTTTTAGTGTTTC
TGATACTCATCTGCATTCTAATTGTAATGATTTTAAGACATAAACAAAAAGACACAATAAACAATTAT
GAGGAGAAGAAAACCTCATCTTTAGATGCGGACTTGAGAGTGACCCGGGATGCCAGTGTGCTCAAAGC
CTTCCAGAAAACTGACGACTGCAGTAACGAGGTGGTCCCTGTGGATGCCACTCCGGAATGGTTGAGTT
TAATAAGTATCATGGAGAAGGATATTGTCAATCTGTACAGACACTCAAACTCCAGTGGCCACTGTTCT
GTGGAAGGAGAAACTGCAGAAGATAAGGAAATCCAGAGGATAAATGAGCATCCCTACAGAAAGTGCTC
AGACTCAGCTCTGAGTGACCACGAGTCCAGGGTGCCAGACTCGGGTATCCCGAGGGACTCAGACCAGC
TCTCCTGCCTATCTGGGGAAACTGATGTGATGGTGACTGCCGAAACAGCAGAAGCCAGCCAAACATTT
GGGGAAGGAGATCAAGGGGAAGGCTGCAGCACCACCTGTGCTCAAAATAATGTGTTACCCCAGACAGT
TCAGAAGAGAGAGGCAAAAGAGAGCATCCTGGCTGATGTTAGAAAAGAGTCTGTCTTTATTTCAGGTG
ATCAGGAAGTAAGGTGTGCAGCTCTTTCAACTCAGACGACCTCTGATCATGATGGAAAGGACAACTAT
CACTGGAATTATCTTCTTAGTTGGGAGCCCAAATTCCAACCTCTTGCCTCAGTATTTAATGATATTGC
AAAACTAAAGGATGAACATTTGCATATGCCTGGCATTCCAAAAGAGAAGAAATCTTTTGTTTTTCCAC
CCCCTTTGATAACAGCAGTAGCCCAGCCTGGGATTAAAGCAGTCCCACCAAGAATGCCGGCAGTAAAC
CTGGGGCAGGTGCCTCCGAAACACCCACGCTCTCCCATCCCCTACCATCTTGGTTCTCTGCCAGAAGG
CATGACTCCCAATTTTTCTCCATCTCTTTCCCTATTGACGATGCAGCCTCCTGCCTTGTCTCCACTGT
TGAGAGAAGGAGAATTATTAGGAACACACATCAGTGGTACATGCCATGAACTTAAAGCAGAAGATGAA
GTTCAAATAGTCGAC

| | | | |
|---|---|---|---|
| NOV50b, 220087646 | SEQ ID NO: 1068 | 1025 aa | MW at 113052.6kD |
| Protein Sequence | | | |

GSEVMTISEDSLPGVIVTHVSVHDVDLNSAFIFSFAKESNPGTKFAIDQNTGVVVLVKTLDFEEMTEY
ELLIQISDSVHYTEGALVVRVLDVNDNPPVFSQDFYQVTVPESIPVGYSVLTLSATDLESNENISYRI
LSSSKEFSIDPKNGTIFTISPVLLLDTISTTQFLVEASDGGNPDLRALTLVEIGIEDMNNYAPEFTVK
SYNLSLSEDALVGSTLVTFSNIDHDWTRENTYVEYSIISGNSQNNFHVETKFFHSEYPYKQVGYLVLL
HSLDREASASHELVILASDSGCPPLSSTAVISIQVLDVNDNPPNFSSLSYHTHVKESTPLGSHITVVS
ANDRDTGSHAEIIYNIISGNEKGHFYLEENTGVLYLIKPLDYEKMTKFTLTVQASDAEKKHFSFAVVF
VSVLDDNDHAPQFMFSSFSCIVPENLPISSTIRSINALDFDAGPYGELTYSIVSPCFLTHGMSYDHDL
FLIDPLTGDIHAKQILDYENGNKYCLTVQAKDKGDATASLVVWVDIEGIDEFEPIFTQDQYFFTLPEK
NKDRQLIGRVEASDADAGIDGVILYSLGTSSPFFSVNKTNGNIYLIRALPLIKSQLNKEDTLEMKIIA
HSPKSDSKFASCTVFVNVSFSSEGTPLAVFASSFSISLVVSFLVFLILICILIVMILRHKQKDTINNY
EEKKTSSLDADLRVTRDASVLKAFQKTDDCSNEVVPVDATPEWLSLISIMEKDIVNLYRHSNSSGHCS
VEGETAEDKEIQRINEHPYRKCSDSALSDHESRVPDSGIPRDSDQLSCLSGETDVMVTAETAEASQTF
GEGDQGEGCSTTCAQNNVLPQTVQKREAKESILADVRKESVFISGDQEVRCAALSTQTTSDHDGKDNY
HWNYLLSWEPKFQPLASVFNDIAKLKDEHLHMPGIPKEKKSFVFPPPLITAVAQPGIKAVPPRMPAVN
LGQVPPKHPRSPIPYHLGSLPEGMTPNFSPSLSLLTMQPPALSPLLREGELLGTHISGTCHELKAEDE
VQIVD

| | | |
|---|---|---|
| NOV50c, 194877960 | SEQ ID NO: 1069 | 3075 bp |
| DNA Sequence | ORF Start: at 1 | ORF Stop: end of sequence j |

GGATCCGAAGTCATGACTATTTCAGAAGATTCTTTGCCTGGTGTAATTGTGACTCATGTGTCAGTTCA
TGATGTGGATTTGAATTCAGCTTTCATATTCAGTTTTGCCAAAGAGAGTAATCCTGGAACCAAGTTTG
CTATTGATCAGAACACTGGAGTGGTGGTGTTGGTGAAAACATTGGATTTTGAAGAAATGACTGAATAT
GAGCTGCTCATCCAAATTTCTGATTCAGTGCACTACACAGAGGGAGCACTTGTAGTCCGTGTAATGGA
TGTCAATGATAATCCACCAGTGTTTTCTCAAGATTTCTATCAGGTCACAGTTCCTGAATCAATACCTG
TGGGGTATTCAGTGCTGACTCTGTCAGCCACAGACTTAGAAAGCAATGAGAACATTTCTTACAGAATT
CTATCCTCTTCTAAGGAATTCTCCATTGATCCTAAGAATGGCACAATATTTACTATCAGCCCCGTATT
ACTTCTGGATACAATATCAACAACTCAATTTCTTGTGGAAGCCAGTGATGGTGGAAATCCCGACCTGA
GAGCTCTTACTTTAGTGGAGATAGGAATAGAAGATATGAACAATTATGCCCCTGAATTCACAGTCAAA
TCCTATAATCTTAGCCTAAGTGAGGATGCTCTGGTTGGAAGCACGCTTGTTACATTTTCAAACATCGA
CCATGACTGGACCCGTGAAAACACATATGTTGAATATTCCATCATCAGTGGTAATTCACAGAACAATT
TTCATGTGGAAACTAAGTTCTTTCATTCAGAATATCCTTATAAGCAAGTCGGTTATCTTGTGTTGCTT
CACAGTCTGGACAGAGAAGCAAGTGCTAGCCATGAGCTTGTCATTCTGGCATCTGACAGTGGCTGCCC
TCCATTGAGTTCCACAGCTGTCATATCAATACAAGTACTTGATGTCAATGACAATCCCCCAAACTTCA
GCAGCCTGAGCTATCACACCCATGTCAAGGAAAGCACCCCTCTAGGGAGTCACATCACTGTGGTCTCA
GCAAATGACCGTGACACAGGGTCACATGCAGAAATCATCTACAACATCATCTCTGGAAATGAGAAGGG
ACATTTTTACTTAGAAGAAAACACTGGAGTTCTTTATTTGATTAAACCTCTGGATTATGAAAAAATGA
CAAAATTCACCTTAACTGTCCAAGCTTCAGATGCAGAAAAGAAACATTTTTCTTTTGCAGTTGTGTTT
GTCAGTGTCCTGGATGATAACGACCATGCACCTCAGTTTATGTTCTCAAGCTTCAGCTGTATTGTTCC
AGAAAATCTGCCTATTTCCTCTACCATATGCTCTATAAATGCTCTGGATTTTGATGCTGGTCCGTATG
GAGAATTGACCTATTCTATTGTATCACCCTGTTTTCTCACTCATGGAATGTCTTATGATCATGATCTC
TTCCTCATTGACCCTTTGACAGGGGATATTCATGCTAAGCAAATCCTTGACTATGAAAATGGCAATAA
ATACTGCCTCACAGTCCAAGCCAAAGACAAAGGTGATGCAACTGCCTCCTTAGTGGTCTGGGTGGATA
TTGAAGGGATAGATGAATTTGAGCCCATTTTCACTCAAGATCAGTATTTTTTCACCCTCCCAGAAAAG
AATAAAGACAGACAGTTGATTGGCAGAGTGGAAGCCTCAGATGCAGATGCTGGTATTGATGGAGTCAT
TCTTTACTCCCTTGGAACCTCATCTCCTTTCTTTTCAGTAAATAAAACCAATGGAAATATTTATTTGA
TTAGAGCCCTTCCCCTAATAAAAAGTCAACTCAACAAAGAAGACACCTTGGAAATGAAAATAATCGCT
CATAGTCCCAAATCAGATTCCAAGTTTGCATCTTGCACTGTTTTTGTGAATGTGTCTTTCTCCTCTGA
AGGAACACCCTTGGCAGTGTTCGCCAGCAGCTTTTCAATCAGCCTGGTGGTCTCCTTTTTAGTGTTTC
TGATACTCATCTGCATTCTAATTGTAATGATTTTAAGACATAAACAAAAAGACACAATAAACAATTAT
GAGGAGAAGAAAACCTCATCTTTAGATGCGGACTTGAGAGTGACCCGGGATGCCAGTGTGCTCAkAGC
CTTCCAGAAAACTGACGACTGCAGTAACGAGGTGGTCCCTGTGGATGCCACTCCGGAATGGTTGAGTT
TAATAAGTATCATGGAGAAGGATATTGTCAATCTGTACAGACACTCAAACTCCAGTGGCCACTGTTCT
GTGGAAGGAGAAACTGCAGAAGATAAGGAAATCCAGAGGATAAATGAGCATCCCTACAGAAAGTGCTC
AGACTCAGCTCTGAGTGACCACGAGTCCAGGGTGCCAGACTCGGGTATCCCGAGGGACTCAGACCAGC
TCTCCTGCCTATCTGGGGAAACTGATGTGATGGTGACTGCCGAAACAGCAGAAGCCAGCCAAACATTT
GGGGAAGGAGATCAAGGGGAAGGCTGCAGCACCACCTGTGCTCAAAATAATGTGTTACCCCAGACAGT
TCAGAAGAGAGAGGCAAAAGAGAGCATCCTGGCTGATGTTAGAAAAGAGTCTGTCTTTATTTCAGGTG
ATCAGGAAGTAAGGTGTGCAGCTCTTTCAACTCAGACGACCTCTGATCATGATGGAAAAGACAACTAT
CACTGGAATTATCTTCTTAGTTGGGAGCCCAAATTCCAACCTCTTGCCTCAGTATTTAATGATATTGC
AAAACTAAAGGATGAACATTTGCATATGCCTGGCATTCCAAAAGAGAAGAAATCTTTTGTTTTTCCAC
CCCCTTTGATAACAGCAGTAGCCCAGCCTGGGATTAAAGCAGTCCCACCAAGAATGCCGGCAGTAAAC
CTGGGGCAGGTGCCTCCGAAACACCCACGCTCTCCCATCCCCTACCATCTTGGTTCTCTGCCAGAAGG
CATGACTCCCAATTTTTCTCCATCTCTTTCCCTATTGACGATGCAGCCTCCTGCCTTGTCTCCACTGT
TGAGAGAAGGAGAATTATTAGGAACACACATCAGTGGTACATGCCATGAACTTAAAGCAGAAGATGAA
GTTCAAATAGTCGAC

| | | | |
|---|---|---|---|
| NOV50c, 194877960 | SEQ ID NO: 1070 | 1025 aa | MW at 112999.6kD |
| Protein Sequence | | | |

GSEVMTTSEDSLPGVTVTHVSVHDVDLNSAFIFSFAKESNPGTKFAIDQNTGVVVLVKTLDFEEMTEY
ELLIQISDSVHYTEGALVVRVLDVNDNPPVFSQDFYQVTVPESIPVGYSVLTLSATDLESNENISYRI
LSSSKEFSIDPKNGTIFTISPVLLLDTISTTQFLVEASDGGNPDLRALTLVEIGIEDMNNYAPEFTVK
SYNLSLSEDALVGSTLVTFSNIDHDWTRENTYVEYSIISGNSQNNFHVETKFFHSEYPYKQVGYLVLL
HSLDREASASHELVILASDSGCPPLSSTAVISIQVLDVNDNPPNFSSLSYHTHVKESTPLGSHITVVS
ANDRDTGSHAEIIYNIISGNEKGHFYLEENTGVLYLIKPLDYEKMTKFTLTVQASDAEKKHFSFAVVF
VSVLDDNDHAPQFMFSSFSCIVPENLPISSTICSINALDFDAGPYGELTYSIVSPCFLTHGMSYDHDL
FLIDPLTGDIHAXQILDYENGNKYCLTVQAKDKGDATASLVVWVDIEGIDEFEPIFTQDQYFFTLPEK
NKDRQLIGRVEASDADAGIDGVILYSLGTSSPFFSVNKTNGNIYLIRALPLIKSQLNKEDTLEMKIIA
HSPKSDSKFASCTVFVNVSFSSEGTPLAVFASSFSISLVVSFLVFLILICILIVMILRHKQKDTINNY
EEKKTSSLDADLRVTRDASVLKAFQKTDDCSNEVVPVDATPEWLSLISIMEKDIVNLYRHSNSSGHCS
VEGETAEDKEIQRINEHPYRKCSDSALSDHESRVPDSGIPRDSDQLSCLSGETDVMVTAETAEASQTF
GEGDQGEGCSTTCAQNNVLPQTVQKREAKESILADVRKESVFISGDQEVRCAALSTQTTSDHDGKDNY
HWNYLLSWEPKFQPLASVFNDIAKLKDEHLHMPGIPKEKKSFVFPPPLITAVAQPGIKAVPPRNPAVN
LGQVPPKHPRSPIPYHLGSLPEGMTPNFSPSLSLLTMQPPALSPLLREGELLGTHISGTCHELKAEDE
VQIVD

| | | |
|---|---|---|
| NOV50d, 192589297 | SEQ ID NO: 1071 | 1899 bp |
| DNA Sequence | ORF Start: at 1 | ORF Stop: end of sequence |

GGATCCGAAGTCATGACTATTTCAGAAGATTCTTTGCCTGGTGTAATTGTGACTCATGTGTCAGTTCA
TGATGTGGATTTGAATTCAGCTTTCATATTCAGTTTTGCCAAAGAGAGTAATCCTGGAACCAAGTTTG
CTATTGATCAGAACACTGGAGTGGTGGTGTTGGTGAAAACATTGGATTTTGAAGAAATGACTGAATAT
GAGCTGCTCATCCAAATTTCTGATTCAGTGCACTACACAGAGGGAGCACTTGTAGTCCGTGTGCTGGA
TGTCAATGATAATCCACCAGTGTTTTCTCAAGATTTCTATCAGGTCACAGTTCCTGAATCAATACCTG
TGGGGTATTCAGTGCTGACTCTGTCAGCCACAGACTTAGAAAGCAATGAGAACATTTCTTACAGAATT
CTATCCTCTTCTAAGGAATTCTCCATTGATCCTAAGAATGGCACAATATTTACTATCAGTCCCGTATT
ACTTCTGGATACAATATCAACAACTCAATTTCTTGTGGAAGCCAGTGATGGTGGAAATCCTGACCTGA
GAGCTCTTACTTTAGTGGAGATAGGAATAGAAGATATGAACAATTATGCCCCTGAATTCACAGTCAAA
TCCTATAATCTTAGCCTAAGTGAGGATGCTCTGGTTGGAAGCACGCTTGTTACATTTTCAAACATCGA
CCATGACTGGACCCGTGAAAACACATATGTTGAATATTCCATCATCAGTGGTAATTCACAGAACAATT
TTCATGTGGAAACTAAGTTCTTTCATTCAGAATATCCTTATAAGCAAGTCGGTTATCTTGTGTTGCTT
CACAGTCTGGACAGAGAAGCAAGTGCTAGCCATGAGCTTGTCATTCTGGCATCTGACAGTGGCTGCCC
TCCATTGAGTTCCACAGCTGTCATATCAATACAAGTACTTGATGTCAATGACAATCCCCCAAACTTCA
GCAGCCTGAGCTATCACACCCATGTCAAGGAAAGCACCCCTCTAGGGAGTCACATCACTGTGGTCTCA
GCAAATGACCGTGACACAGGGTCACATGCAGAAATCATCTACAACATCATCTCTGGAAATGAGAAGGG
ACATTTTTACTTAGAAGAAAACACTGGAGTTCTTTATTTGATTAAACCTCTGGATTATGAAAAAATGA
CAAAATTCACCTTAACTGTCCAAGCTTCAGATGCAGAAAAGAAACATTTTTCTTTTGCAGTTGTGTTT
GTCAGTGTCCTGGATGATAACGACCATGCACCTCAGTTTATGTTCTCAAGCTTCAGCTGTATTGTTCC
AGAAAATCTGCCTATTTCCTCTACCATATGCTCTATAAATGCTCTGGATTTTGATGCTGGTCCGTATG
GAGAATTGACCTATTCTATTGTATCACCCTGTTTTCTCACTCATGGAATGTCTTATGATCATGATCTC
TTCCTCATTGACCCTTTGACAGGGGATATTCATGCTAAGCAAATCCTTGACTATGAAAATGGCAATAA
ATACTGCCTCACAGTCCAAGCCAAAGACAAAGGTGATGCAACTGCCTCCTTAGTGGTCTGGGTGGATA
TTGAAGGGATAGATGAATTTGAGCCCATTTTCACTCAAGATCAGTATTTTTTCACCCTCCCAGAAAAG
AATAAAGACAGACAGTTGATTGGCAGAGTGGAAGCCTCAGATGCAGATGCTGGTATTGATGGAGTCAT
TCTTTACTCCCTTGGAACCTCATCTCCTTTCTTTTCAGTAAATAAAACCAATGGAAATATTTATTTGA
TTAGAGCCCTTCCCCTAATAAAAAGTCAACTCAACAAAGAAGACACCTTGGAAATGAAAATAATCGCT
CATAGTCCCAAATCAGATTCCAAGTTTGCATCTTGCACTGTTTTTGTGAATGTGTCTGTCGAC

| | | | |
|---|---|---|---|
| NOV50d, 192589297 | SEQ ID NO: 1072 | 633 aa | MW at 69903.4kD |
| Protein Sequence | | | |

GSEVMTISEDSLPGVIVTHVSVHDVDLNSAFIFSFAKESNPGTKFAIDQNTGVVVLVKTLDFEEMTEY
ELLIQISDSVHYTEGALVVRVLDVNDNPPVFSQDFYQVTVPESIPVGYSVLTLSATDLESNENISYRI
LSSSKEFSIDPKNGTIFTISPVLLLDTISTTQFLVEASDGGNPDLRALTLVEIGIEDMNNYAPEFTVK
SYNLSLSEDALVGSTLVTFSNIDHDWTRENTYVEYSIISGNSQNNFHVETKFFHSEYPYKQVGYLVLL
HSLDREASASHELVILASDSGCPPLSSTAVISIQVLDVNDNPPNFSSLSYHTHVKESTPLGSHITVVS
ANDRDTGSHAEIIYNIISGNEKGHFYLEENTGVLYLIKPLDYEKMTKFTLTVQASDAEKKHFSFAVVF
VSVLDDNDHAPQFMFSSFSCIVPENLPISSTICSINALDFDAGPYGELTYSIVSPCFLTHGMSYDHDL
FLIDPLTGDIHAKQILDYENGNKYCLTVQAKDKGDATASLVVWVDIEGIDEFEPIFTQDQYFFTLPEK
NKDRQLIGRVEASDADAGIDGVILYSLGTSSPFFSVNKTNGNIYLIRALPLIKSQLNKEDTLEMKIIA
HSPKSDSKFASCTVFVNVSVD

| | | |
|---|---|---|
| NOV50e, CG58567-02 | SEQ ID NO: 1073 | 2956 bp |
| DNA Sequence | ORF Start: ATG at 57 | ORF Stop: TGA at 2934 |

CTATTGATCAGAACACTGGAGTGGTGGTGTTGGTGAAAACATTGGATTTTGAAGAAATGACTGAATAT
GAGCTGCTCATCCAAATTTCTGATTCAGTGCACTACACAGAGGGAGCACTTGTAGTCCGTGTGCTGGA
TGTCAATGATAATCCACCAGTGTTTTCTCAAGATTTCTATCAGGTCACAGTTCCTGAATCAATACCTG
TGGGGTATTCAGTGCTGACTCTGTCAGCCACAGACTTAGAAAGCAATGAGAACATTTCTTACAGAATT
CTATCCTCTTCTAAGGAATTCTCCATTGATCCTAAGAATGGCACAATATTTACTATCAGTCCCGTATT
ACTTCTGGATACAATATCAACAACTCAATTTCTTGTGGAAGCCAGTGATGGTGGAAATCCTGACCTGA
GAGCTCTTACTTTAGTGGAGATAGGAATAGAAGATATGAACAATTATGCCCCTGAATTCACAGTCAAA
TCCTATAATCTTAGCCTAAGTGAGGATGCTCTGGTTGGAAGCACGCTTGTTACATTTTCAAACATCGA
CCATGACTGGACCCGTGAAAACACATATGTTGAATATTCCATCATCAGTGGTAATTCACAGAACAATT
TTCATGTGGAAACTAAGTTCTTTCATTCAGAATATCCTTATAAGCAAGTCGGTTATCTTGTGTTGCTT
CACAGTCTGGACAGAGAAGCAAGTGCTAGCCATGAGCTTGTCATTCTGGCATCTGACAGTGGCTGCCC
TCCATTGAGTTCCACAGCTGTCATATCAATACAAGTACTTGATGTCAATGACAATCCCCCAAACTTCA
GCAGCCTGAGCTATCACACCCATGTCAAGGAAAGCACCCCTCTAGGGAGTCACATCACTGTGGTCTCA
GCAAATGACCGTGACACAGGGTCACATGCAGAAATCATCTACAACATCATCTCTGGAAATGAGAAGGG
ACATTTTTACTTAGAAGAAAACACTGGAGTTCTTTATTTGATTAAACCTCTGGATTATGAAAAAATGA
CAAAATTCACCTTAACTGTCCAAGCTTCAGATGCAGAAAAGAAACATTTTTCTTTTGCAGTTGTGTTT
GTCAGTGTCCTGGATGATAACGACCATGCACCTCAGTTTATGTTCTCAAGCTTCAGCTGTATTGTTCC
AGAAAATCTGCCTATTTCCTCTACCATATGCTCTATAAATGCTCTGGATTTTGATGCTGGTCCGTATG
GAGAATTGACCTATTCTATTGTATCACCCTGTTTTCTCACTCATGGAATGTCTTATGATCATGATCTC
TTCCTCATTGACCCTTTGACAGGGGATATTCATGCTAAGCAAATCCTTGACTATGAAAATGGCAATAA
ATACTGCCTCACAGTCCAAGCCAAAGACAAAGGTGATGCAACTGCCTCCTTAGTGGTCTGGGTGGATA
TTGAAGGGATAGATGAATTTGAGCCCATTTTCACTCAAGATCAGTATTTTTTCACCCTCCCAGAAAAG
AATAAAGACAGACAGTTGATTGGCAGAGTGGAAGCCTCAGATGCAGATGCTGGTATTGATGGAGTCAT
TCTTTACTCCCTTGGAACCTCATCTCCTTCCCTTTCAGTAAATAAAACCAATGGAAATATTTATTTGA
TTAGAGCCCTTCCCCTAATAAAAAGTCAACTCAACAAAGAAGACACCTTGGAAATGAAAATAATCGCT
CATAGTCCCAAATCAGATTCCAAGTTTGCATCTTGCACTGTTTTTGTGAATGTGTCTTTCTCCTCTGA
AGGAACACCCTTGGCAGTGTTCGCCAGCAGCTTTTCAATCAGCCTGGTGGTCTCCTTTTTAGTGTTTC
TGATACTCATCTGCATTCTAATTGTAATGATTTTAAGACATAAACAAAAAGACACAATAAACAATTAT
GAGGAGAAGAAAACCTCATCTTTAGATGCGGACTTGAGAGTGACCCGGGATGCCAGTGTGCTCAAAGC
CTTCCAGAAAACTGACGACTGCAGTAACGAGGTGGTCCCTGTGGATGCCACTCCGGAATGGTTGAGTT
TAATAAGTATCATGGAGAAGGATATTGTCAATCTGTACAGACACTCAAACTCCAGTGGCCACTGTTCT
GTGGAAGGAGAAACTGCAGAAGATAAGGAAATCCAGAGGATAAATGAGCATCCCTACAGAAAGTGCTC
AGACTCAGCTCTGAGTGACCACGAGTCCAGGGTGCCAGACTCGGGTATCCCGAGGGACTCAGACCAGC
TCTCCTGCCTATCTGGGGAAACTGATGTGATGGTGACTGCCGAAACAGCAGAAGCCAGCCAAACATTT
GGGGAAGGAGATCAAGGGGAAGGCTGCAGCACCACCTGTGCTCAAAATAATGTGTTACCCCAGACAGT
TCAGAAGAGAGAGGCAAAAGAGAGCATCCTGGCTGATGTTAGAAAAGAGTCTGTCTTTATTTCAGGTG
ATCAGGAAGTAAGGTGTGCAGCTCTTTCAACTCAGACGACCTCTGATCATGATGGAAAAGACAACTAT
CACTGGAATTATCTTCTTAGTTGGGAGCCCAAATTCCAACCTCTTGCCTCAGTATTTAATGATATTGC
AAAACTAAAGGATGAACATTTGCATATGCCTGGCATTCCAAAAGAGAAGAAATCTTTTGTTTTTCCAC
CCCCTTTGATAACAGCAGTAGCCCAGCCTGGGATTAAAGCAGTCCCACCAAGAATGCCGGCAGTAAAC
CTGGGGCAGGTGCCTCCGAAACACCCACGCTCTCCCATCCCCTACCATCTTGGTTCTCTGCCAGAAGG
CATGACTCCCAATTTTTCTCCATCTCTTTCCCTATTGACGATGCAGCCTCCTGCCTTGTCTCCACTGT
TGAGAGAAGGAGAATTATTAGGAACACACATCAGTGGTACATGCCATGAACTTAAAGCAGAAGATGAA
GTTCAAATATGAAACCACTGGGATGCCAAGTA

| | | | |
|---|---|---|---|
| NOV50e, CG58567-02 | SEQ ID NO: 1074 | 959 aa | MW at 105826.6kD |
| Protein Sequence | | | |

MTEYELLIQISDSVHYTEGALVVRVLDVNDNPPVFSQDFYQVTVPESIPVGYSVLTLSATDLESNENI
SYRILSSSKEFSIDPKNGTIFTISPVLLLDTISTTQFLVEASDGGNPDLRALTLVEIGIEDMNNYAPE
FTVKSYNLSLSEDALVGSTLVTFSNIDHDWTRENTYVEYSIISGNSQNNFHVETKFFHSEYPYKQVGY
LVLLHSLDREASASHELVILASDSGCPPLSSTAVISIQVLDVNDNPPNFSSLSYHTHVKESTPLGSHI
TVVSANDRDTGSHAEIIYNIISGNEKGHFYLEENTGVLYLIKPLDYEKMTKFTLTVQASDAEKKHFSF
AVVFVSVLDDNDHAPQFMFSSFSCIVPENLPISSTICSINALDFDAGPYGELTYSIVSPCFLTHGMSY
DHDLFLIDPLTGDIHAKQILDYENGNKYCLTVQAKDKGDATASLVVWVDIEGIDEFEPIFTQDQYFFT
LPEKNKDRQLIGRVEASDADAGIDGVILYSLGTSSPSLSVNKTNGNIYLIRALPLIKSQLNKEDTLEM
KIIAHSPKSDSKFASCTVFVNVSFSSEGTPLAVFASSFSISLVVSFLVFLILICILIVMILRHKQKDT
INNYEEKKTSSLDADLRVTRDASVLKAFQKTDDCSNEVVPVDATPEWLSLISIMEKDIVNLYRHSNSS
GHCSVEGETAEDKEIQRINEHPYRKCSDSALSDHESRVPDSGIPRDSDQLSCLSGETDVMVTAETAEA
SQTFGEGDQGEGCSTTCAQNNVLPQTVQKREAKESILADVRKESVFISGDQEVRCAALSTQTTSDHDG
KDNYHWNYLLSWEPKFQPLASVFNDIAKLKDEHLHMPGIPKEKKSFVFPPPLITAVAQPGIKAVPPRM
PAVNLGQVPPKHPRSPIPYHLGSLPEGMTPNFSPSLSLLTMQPPALSPLLREGELLGTHISGTCHELK
AEDEVQI

| | | |
|---|---|---|
| NOV50f, CG58567-03 | SEQ ID NO: 1075 | 3075 bp |
| DNA Sequence | ORF Start: at 7 | ORF Stop: at 3070 |

GGATCCGAAGTCATGACTATTTCAGAAGATTCTTTGCCTGGTGTAATTGTGACTCATGTGTCAGTTCA
TGATGTGGATTTGAATTCAGCTTTCATATTCAGTTTTGCCAAAGAGAGTAATCCTGGAACCAAGTTTG
CTATTGATCAGAACACTGGAGTGGTGGTGTTGGTGAAAACATTGGATTTTGAAGAAATGACTGAATAT
GAGCTGCTCATCCAAATTTCTGATTCAGTGCACTACACAGAGGGAGCACTTGTAGTCCGTGTGCTGGA
TGTCAATGATAATCCACCAGTGTTTTCTCAAGATTTCTATCAGGTCACAGTTCCTGAATCAATACCTG
TGGGGTATTCAGTGCTGACTCTGTCAGCCACAGACTTAGAAAGCAATGAGAACATTTCTTACAGAATT
CTATCCTCTTCTAAGGAATTCTCCATTGATCCTAAGAATGGCACAATATTTACTATCAGCCCCGTATT
ACTTCTGGATACAATATCAACAACTCAATTTCTTGTGGAAGCCAGTGATGGTGGAAATCCCGACCTGA
GAGCTCTTACTTTAGTGGAGATAGGAATAGAAGATATGAACAATTATGCCCCTGAATTCACAGTCAAA
TCCTATAATCTTAGCCTAAGTGAGGATGCTCTGGTTGGAAGCACGCTTGTTACATTTTCAAACATCGA
CCATGACTGGACCCGTGAAAACACATATGTTGAATATTCCATCATCAGTGGTAATTCACAGAACAATT
TTCATGTGGAAACTAAGTTCTTTCATTCAGAATATCCTTATAAGCAAGTCGGTTATCTTGTGTTGCTT
CACAGTCTGGACAGAGAAGCAAGTGCTAGCCATGAGCTTGTCATTCTGGCATCTGACAGTGGCTGCCC
TCCATTGAGTTCCACAGCTGTCATATCAATACAAGTACTTGATGTCAATGACAATCCCCCAAACTTCA
GCAGCCTGAGCTATCACACCCATGTCAAGGAAAGCACCCCTCTAGGGAGTCACATCACTGTGGTCTCA
GCAAATGACCGTGACACAGGGTCACATGCAGAAATCATCTACAACATCATCTCTGGAAATGAGAAGGG
ACATTTTTACTTAGAAGAAAACACTGGAGTTCTTTATTTGATTAAACCTCTGGATTATGAAAAAATGA
CAAAATTCACCTTAACTGTCCAAGCTTCAGATGCAGAAAAGAAACATTTTTCTTTTGCAGTTGTGTTT
GTCAGTGTCCTGGATGATAACGACCATGCACCTCAGTTTATGTTCTCAAGCTTCAGCTGTATTGTTCC
AGAAAATCTGCCTATTTCCTCTACCATATGCTCTATAAATGCTCTGGATTTTGATGCTGGTCCGTATG
GAGAATTGACCTATTCTATTGTATCACCCTGTTTTCTCACTCATGGAATGTCTTATGATCATGATCTC
TTCCTCATTGACCCTTTGACAGGGGATATTCATGCTAAGCAAATCCTTGACTATGAAAATGGCAATAA
ATACTGCCTCACAGTCCAAGCCAAAGACAAAGGTGATGCAACTGCCTCCTTAGTGGTCTGGGTGGATA
TTGAAGGGATAGATGAATTTGAGCCCATTTTCACTCAAGATCAGTATTTTTTCACCCTCCCAGAAAAG
AATAAAGACAGACAGTTGATTGGCAGAGTGGAAGCCTCAGATGCAGATGCTGGTATTGATGGAGTCAT
TCTTTACTCCCTTGGAACCTCATCTCCTTTCTTTTCAGTAAATAAAACCAATGGAAATATTTATTTGA
TTAGAGCCCTTCCCCTAATAAAAAGTCAACTCAACAAAGAAGACACCTTGGAAATGAAAATAATCGCT
CATAGTCCCAAATCAGATTCCAAGTTTGCATCTTGCACTGTTTTTGTGAATGTGTCTTTCTCCTCTGA
AGGAACACCCTTGGCAGTGTTCGCCAGCAGCTTTTCAATCAGCCTGGTGGTCTCCTTTTTAGTGTTTC
TGATACTCATCTGCATTCTAATTGTAATGATTTTAAGACATAAACAAAAAGACACAATAAACAATTAT
GAGGAGAAGAAAACCTCATCTTTAGATGCGGACTTGAGAGTGACCCGGGATGCCAGTGTGCTCAAAGC
CTTCCAGAAAACTGACGACTGCAGTAACGAGGTGGTCCCTGTGGATGCCACTCCGGAATGGTTGAGTT
TAATAAGTATCATGGAGAAGGATATTGTCAATCTGTACAGACACTCAAACTCCAGTGGCCACTGTTCT
GTGGAAGGAGAAACTGCAGAAGATAAGGAAATCCAGAGGATAAATGAGCATCCCTACAGAAAGTGCTC
AGACTCAGCTCTGAGTGACCACGAGTCCAGGGTGCCAGACTCGGGTATCCCGAGGGACTCAGACCAGC
TCTCCTGCCTATCTGGGGAAACTGATGTGATGGTGACTGCCGAAACAGCAGAAGCCAGCCAAACATTT
GGGGAAGGAGATCAAGGGGAAGGCTGCAGCACCACCTGTGCTCAAAATAATGTGTTACCCCAGACAGT
TCAGAAGAGAGAGGCAAAAGAGAGCATCCTGGCTGATGTTAGAAAAGAGTCTGTCTTTATTTCAGGTG
ATCAGGAAGTAAGGTGTGCAGCTCTTTCAACTCAGACGACCTCTGATCATGATGGAAAAGACAACTAT
CACTGGAATTATCTTCTTAGTTGGGAGCCCAAATTCCAACCTCTTGCCTCAGTATTTAATGATATTGC
AAAACTAAAGGATGAACATTTGCATATGCCTGGCATTCCAAAAGAGAAGAAATCTTTTGTTTTTCCAC
CCCCTTTGATAACAGCAGTAGCCCAGCCTGGGATTAAAGCAGTCCCACCAAGAATGCCGGCAGTAAAC
CTGGGGCAGGTGCCTCCGAAACACCCACGCTCTCCCATCCCCTACCATCTTGGTTCTCTGCCAGAAGG
CATGACTCCCAATTTTTCTCCATCTCTTTCCCTATTGACGATGCAGCCTCCTGCCTTGTCTCCACTGT
TGAGAGAAGGAGAATTATTAGGAACACACATCAGTGGTACATGCCATGAACTTAAAGCAGAAGATGAA
GTTCAAATAGTCGAC

| | | | |
|---|---|---|---|
| NOV50f, CG58567-03 | SEQ ID NO: 1076 | 1021 aa | MW at 112641.2kD |
| Protein Sequence | | | |

EVMTISEDSLPGVIVTHVSVHDVDLNSAFIFSFAKESNPGTKFAIDQNTGVVVLVKTLDFEEMTEYEL
LIQISDSVHYTEGALVVRVLDVNDNPPVFSQDFYQVTVPESIPVGYSVLTLSATDLESNENISYRILS
SSKEFSIDPKNGTIFTISPVLLLDTISTTQFLVEASDGGNPDLRALTLVEIGIEDMNNYAPEFTVKSY
NLSLSEDALVGSTLVTFSNIDHDWTRENTYVEYSIISGNSQNNFHVETKFFHSEYPYKQVGYLVLLHS
LDREASASHELVILASDSGCPPLSSTAVISIQVLDVNDNPPNFSSLSYHTHVKESTPLGSHITVVSAN
DRDTGSHAEIIYNIISGNEKGHFYLEENTGVLYLIKPLDYEKMTKFTLTVQASDAEKKHFSFAVVFVS
VLDDNDHAPQFMFSSFSCIVPENLPISSTICSINALDFDAGPYGELTYSIVSPCFLTHGMSYDHDLFL
IDPLTGDIHAKQILDYENGNKYCLTVQAKDKGDATASLVVWVDIEGIDEFEPIFTQDQYFFTLPEKNK
DRQLIGRVEASDADAGIDGVILYSLGTSSPFFSVNKTNGNIYLIRALPLIKSQLNKEDTLEMKIIAHS
PKSDSKFASCTVFVNVSFSSEGTPLAVFASSFSISLVVSFLVFLILICILIVMILRHKQKDTINNYEE
KKTSSLDADLRVTRDASVLKAFQKTDDCSNEVVPVDATPEWLSLISIMEKDIVNLYRHSNSSGHCSVE
GETAEDKEIQRINEHPYRKCSDSALSDHESRVPDSGIPRDSDQLSCLSGETDVMVTAETAEASQTFGE
GDQGEGCSTTCAQNNVLPQTVQKREAKESILADVRKESVFISGDQEVRCAALSTQTTSDHDGKDNYHW
NYLLSWEPKFQPLASVFNDIAKLKDEHLHMPGIPKEKKSFVFPPPLITAVAQPGIKAVPPRMPAVNLG
QVPPKHPRSPIPYHLGSLPEGMTPNFSPSLSLLTMQPPALSPLLREGELLGTHISGTCHELKAEDEVQ
I

| | | |
|---|---|---|
| NOV50g, CG58567-04 | SEQ ID NO: 1077 | 1899 bp |
| DNA Sequence | ORF Start: at 1 | ORF Stop: end of sequence |

GGATCCGAAGTCATGACTATTTCAGAAGATTCTTTGCCTGGTGTAATTGTGACTCATGTGTCAGTTCA
TGATGTGGATTTGAATTCAGCTTTCATATTCAGTTTTGCCAAAGAGAGTAATCCTGGAACCAAGTTTG
CTATTGATCAGAACACTGGAGTGGTGGTGTTGGTGAAAACATTGGATTTTGAAGAAATGACTGAATAT
GAGCTGCTCATCCAAATTTCTGATTCAGTGCACTACACAGAGGGAGCACTTGTAGTCCGTGTGCTGGA
TGTCAATGATAATCCACCAGTGTTTTCTCAAGATTTCTATCAGGTCACAGTTCCTGAATCAATACCTG
TGGGGTATTCAGTGCTGACTCTGTCAGCCACAGACTTAGAAAGCAATGAGAACATTTCTTACAGAATT
CTATCCTCTTCTAAGGAATTCTCCATTGATCCTAAGAATGGCACAATATTTACTATCAGTCCCGTATT
ACTTCTGGATACAATATCAACAACTCAATTTCTTGTGGAAGCCAGTGATGGTGGAAATCCTGACCTGA
GAGCTCTTACTTTAGTGGAGATAGGAATAGAAGATATGAACAATTATGCCCCTGAATTCACAGTCAAA
TCCTATAATCTTAGCCTAAGTGAGGATGCTCTGGTTGGAAGCACGCTTGTTACATTTTCAAACATCGA
CCATGACTGGACCCGTGAAAACACATATGTTGAATATTCCATCATCAGTGGTAATTCACAGAACAATT
TTCATGTGGAAACTAAGTTCTTTCATTCAGAATATCCTTATAAGCAAGTCGGTTATCTTGTGTTGCTT
CACAGTCTGGACAGAGAAGCAAGTGCTAGCCATGAGCTTGTCATTCTGGCATCTGACAGTGGCTGCCC
TCCATTGAGTTCCACAGCTGTCATATCAATACAAGTACTTGATGTCAATGACAATCCCCCAAACTTCA
GCAGCCTGAGCTATCACACCCATGTCAAGGAAAGCACCCCTCTAGGGAGTCACATCACTGTGGTCTCA
GCAAATGACCGTGACACAGGGTCACATGCAGAAATCATCTACAACATCATCTCTGGAAATGAGAAGGG
ACATTTTTACTTAGAAGAAAACACTGGAGTTCTTTATTTGATTAAACCTCTGGATTATGAAAAAATGA
CAAAATTCACCTTAACTGTCCAAGCTTCAGATGCAGAAAAGAAACATTTTTCTTTTGCAGTTGTGTTT
GTCAGTGTCCTGGATGATAACGACCATGCACCTCAGTTTATGTTCTCAAGCTTCAGCTGTATTGTTCC
AGAAAATCTGCCTATTTCCTCTACCATATGCTCTATAAATGCTCTGGATTTTGATGCTGGTCCGTATG
GAGAATTGACCTATTCTATTGTATCACCCTGTTTTCTCACTCATGGAATGTCTTATGATCATGATCTC
TTCCTCATTGACCCTTTGACAGGGGATATTCATGCTAAGCAAATCCTTGACTATGAAAATGGCAATAA
ATACTGCCTCACAGTCCAAGCCAAAGACAAAGGTGATGCAACTGCCTCCTTAGTGGTCTGGGTGGATA
TTGAAGGGATAGATGAATTTGAGCCCATTTTCACTCAAGATCAGTATTTTTTCACCCTCCCAGAAAAG
AATAAAGACAGACAGTTGATTGGCAGAGTGGAAGCCTCAGATGCAGATGCTGGTATTGATGGAGTCAT
TCTTTACTCCCTTGGAACCTCATCTCCTTTCTTTTCAGTAAATAAAACCAATGGAAATATTTATTTGA
TTAGAGCCCTTCCCCTAATAAAAAGTCAACTCAACAAAGAAGACACCTTGGAAATGAAAATAATCGCT
CATAGTCCCAAATCAGATTCCAAGTTTGCATCTTGCACTGTTTTTGTGAATGTGTCTGTCGAC

| | | | |
|---|---|---|---|
| NOV50g, CG58567-04 | SEQ ID NO: 1078 | 633 aa | MW at 69903.4kD |
| Protein Sequence | | | |

GSEVMTISEDSLPGVIVTHVSVHDVDLNSAFIFSFAKESNPGTKFAIDQNTGVVVLVKTLDFEEMTEY
ELLIQISDSVHYTEGALVVRVLDVNDNPPVFSQDFYQVTVPESIPVGYSVLTLSATDLESNENISYRI
LSSSKEFSIDPKNGTIFTISPVLLLDTISTTQFLVEASDGGNPDLPALTLVEIGIEDMNNYAPEFTVK
SYNLSLSEDALVGSTLVTFSNIDHDWTRENTYVEYSIISGNSQNNFHVETKFFHSEYPYKQVGYLVLL
HSLDREASASHELVILASDSGCPPLSSTAVISIQVLDVNDNPPNFSSLSYHTHVKESTPLGSHITVVS
ANDRDTGSHAEIIYNIISGNEKGHFYLEENTGVLYLIKPLDYEKMTKFTLTVQASDAEKKHFSFAVVF
VSVLDDNDHAPQFMFSSFSCIVPENLPISSTICSINALDFDAGPYGELTYSIVSPCFLTHGMSYDHDL
FLIDPLTGDIHAKQILDYENGNKYCLTVQAKDKGDATASLVVWVDIEGIDEFEPIFTQDQYFFTLPEK
NKDRQLIGRVEASDADAGIDGVILYSLGTSSPFFSVNKTNGNIYLIRALPLIKSQLNKEDTLEMKIIA
HSPKSDSKFASCTVFVNVSVD

| | | |
|---|---|---|
| NOV50h, CG58567-05 | SEQ ID NO: 1079 | 9193 bp |
| DNA Sequence | ORF Start: ATG at 1 | ORF Stop: TGA at 9010 |

ATGGAGAAATGTGGGCTTAAAGAGGAAGGCAGTTACGCGGACATAGATCCAGTTGCCCACGATCCGGA
CGCCGGACTGTTCAGCACTCAGGGCTACACCCTGGTGCAACCGTCCGACCTGCCCAAGGACCCCGCAG
GCCCGTTCTTCCAGTTGCGCTACCGGACTCCGGGGCCACTACCGTCACCGCTTTTGCCAGGCTCCTCG
TCACCCCTGGAGCCTCTAGATCTGGTGCTGCTGCGGCGCTTGGACCGAGAGGAGGCGGCGGCGCACCG
GCTGCAGATCGAGGCATGGGACGGCGGCCGACCCCGGCGCACCGGCCTCCTGAGCGTGGAGCTGCGCG
TGCTGGATGAGAACGACAACCCGCCGGTCTTTGAGCAGGACGAGTACCGCGCCGCGGTGCGCGAGGAC
GCCCAGCCGGGCGCCGAGGTCTGTCGCGTGCGCGCCACCGACCGCGACCTGGGGCCCAATGGCTTCGT
GCGCTACAGCGTCCGCGCCCGGCAAGTGCCTGGGGCGGGTAGCGGCGGCGGGGCACTGGGCGACGCGG
CCTACTTCGCGGTGGAGGAGCTGAGCGGCGTGGTGCGAGTGTGGAGACCTCTGGACCGCGAGGCACAG
GCCTGGCACCAGTTGGTGGTGGAGGCCCGCGATGGAGGCGCCGAGCCTGAGGTTGCCACGGTGCGCGT
GTCCATCGCCGTGCTGGACGTGAATGACAACCGGCCAGCAATTCACGTGCTCTTTCTCACAGAGGGAG
GCGTCGCCCGTGTCTCTGAAGGCGCCCGACCGGGCGACTACGTGGCTCGCGTCTCGGTGTCTGACGCG
GACGGGGTAATAGGCAAAATTACAGCTATTGACATGGACTCTGGAAAGAATGGACAGCTATTATATTT
CCTTTTGTCTGATGGAAAATTCTTCAAGATGAATCCTAATACAGGAGAGTTAATCAATTGGGTGGCAC
TGGATCGTGAGCACCGGGGGCACCATGAGATGACTGTGCTAGTGACAGACCGCGGCTCCCCACCACGA
AACGCCACCATGGCGGTTTACGTCTCAGTTACTGACATCAATGATAACAGGCCCTTCTTCCCCCAGTG
TCTCCCTGGAAAGGAGTTACACGTGAAGGTTCTGGAAGGTCAACCAGTAAATATGTTGGTTACAACTG
TGTTTGCAAAGGATCCTGATGAAGGAAATAATGCAGAAGTTACATACTCAGTATCTTCAGAAGATAGT
TCTGATCACTTTAAGATTGACGCCAACAATGGTGAAATAAGAACAACCACAATACTTTCGTATGATTA
TAGACCTTCCTACAGAATGAGTGTCATTGCCACTGACCAGGGAGTGCCTCCTCTTCAAGGACAGGCAG
TTGTTAATATTCAGGAACTTGATTATGAGACGACATCTCATTATCTTTTCAGAGTGATTACTACAGAC
CATAGCAAAAACCTTTCCCTGAGTAGCACAGTCTTCCTTAGTATCGATGTGGAAGATCAGAATGACCA
TTCCCCATCTTTCCAGGATGAGCTCATTGTGATCAGTGTAGAGGAGAATGTTCCCATAGGAACCCTGG
TGTATGTCTTCAATGCCAAAGATGATGACGGCAGTTTTTTGAACAGTAGAATACAATACTACATTGAA
TCCCACAACCCTGGCACGAATCCATTTCTCATCCACCCCTCATTTGGCACACTAGTCACTGTGTCCCG
TCTTGACAGAGAAAGCATTCCAACTGTCATCCTGACAGTAACAGCATCTGATCAGGCTGTGAATGTGA
CAGACCGGCGACTGAGATCACTGACAGCACAAATAGTGATTTTGGATGTAAATGACCACAACCCCACT
TTTATTTCTTTCCCCAATGCCCATGTCAAAGAGGATGTCACAGTGGGCTCCTTGGTCCACCACATAAC
TGCTCACGATCCAGACGAAGGAAGGAATGGAAAAGTAACATACAGCATCCTCTCAGGAAATGAAAACA
TGACGTTTATGCTAGATGAGTCATCAGGCTTACTAACCACAACCTGTCCTTTGGATTATGAAATGAAA
ACTCAGCATATTCTGACTGTTCTGGCACTGGATGATGGCACACCAGCACTTTCTTCATCCCAGACTTT
GACAGTTACTGTTCTTGATGTAAATGATGAAGCTCCAGTATTTAAGCAGCACCTGTATGAAGCCTCAG
TGAAAGAAAACCAAAATCCAGGGGAGTTTGTTACCAGGGTTGAAGCTCTGGACAGAGATTCAGTGTTC
CTTAATACTAGAGAGCTTAACATGTGTTTTCTAGCATTCTACGATGCAGTTTTTAAAAATGGTGGGCT
AAGTGCCCAAGCCTTTGTTCGTGTGGACCTGGAGGACGTGAATGATAATCATCCTGTGTTTAACCCAT
CAACCTATGTGACGAGCATCAGTGATGAGACCCAGCCAGGCACCGAGATCATCAATGTTCTTGCCACT
GACCAGGACTCTGGGATATATGGGACAGTGGCTTATGAGCTTATTCCAGGAAACGTGTCGTCCCTTTT
TACCATTGACTCCACCACAGGAATTATTTACTTAACATTACCTCTTAGTCATTTGGAATCTACCACAC
TTTCGTTGATGGTCTCTGCTCAAGACGGTGGTGGGCTCACAGCTGTCATTAATGCCGATGTCACCATA
CACATTTTCCAGACAACTCTGGCACCTGCTGAGTTTGAAAGGCCTAAGTACACTTTCTTAGTTTATGA
AGATGTGCCTGAAGATAGTCCCATTGGAACAGTGAAAGCAAGAGAGCCCTTGAATCCACCTAGGAGCT
CTGTAATACACCTGCAAGTTAGAGTTTTGGATGCCAATGACCACAGTCCTTCTTTTCCCACACTTTAT
TACCAGTCCTCTGTGAGAGAAGATGCTGAAGTGGGAACAGTGGTTCTTGTGCTTTCAGCTGTGGACAA
GGATGAAGGCCTGAATGGGCAAACTGAGTATTTTCTGACTGATGAGGCTTGTGGTGCATTCACCATTG
ATCCTATGTCAGGCACATTGAAAACCAGCAACACCCTCGACCGTGAAGCCAGATCTCAGCATACATTT
AGTGCTGTGGCCAGAGACTGTAGCATCCAGGGTTCACGAAGCACCACTGTAATTATAAAAGTATATGT
CACTGATGTTAATGACAATGATCCAGTTTTGGAACAGAACCCTTTTGATGTGTTTCTTTCCCCCGAGT
CGCCTACAAACCAGACAACTGTCATTGTGAGAGCTGATGACCTGGACTTGGGGCCCAATGGAACTGTG
GACTCCGATGACTCCCCGCTGCTGGACGACTTCCACGTGCACCCGGACACCGGCATCATCCGCACTGC
GCGGCGCCTGGACCGCGAGCGGCGGGACCACTACAGCTTCGTCGCCGCCACGCTGCTGGGCGCTGTGG
TGCAGGTGGAGATTCGCGTCAACGACGTGAATGACCACTCGCCCCGCTTTCCCCTCGACTCCCTGCAA
CTCGACGTCTCCGAGCTCAGCCCGCCAGGGACCGCCTTCCGCCTGCCAGTTGCCCACGATCCGGACGC
CGGACTGTTCAGCACTCAGGGCTACACCCTGGTGCAACCGTCCGACCTGCCCAAGGACCCCGCAGGCC
CGTTCTTCCAGTTGCGCTACCGGACTCCGGGGCCACTACCGTCACCGCTTTTGCCAGGCTCCTCGTCA
CCCCTGGAGCCTCTAGATCTGGTGCTGCTGCGGCGCTTGGACCGAGAGGAGGCGGCGGCGCACCGGCT
GCAGATCGAGGCATGGGACGGCGGCCGACCCCGGCGCACCGGCCTCCTGAGCGTGGAGCTGCGCGTGC
TGGATGAGAACGACAACCCGCCGGTCTTTGAGCAGGACGAGTCCCGCGCCGCGGTGCGAGAGCACGCC
CAGCCGGGCGCCGAGGTCTGTCGCGTGCGCGCCACCGCCCGCGACGTGGGGCCCAATGGCTTCGTGCG
CTACAGCGTCCGCGCCCGGCAAGTGCCTGGGGCGGGTAGCGGCGGCGGGGCACTGGGCGACGCGGCCT
ACTTCGCGTTGGTGGTGGAGGCCCGCGATGGAGGCGCCGAGCATGAGGTTGCCGCGGTGCGTGTGTCC
ATCGCCGTGCTGGACGTGAATGACAACCGGCCAGCAATTCACGTGCTCTTTCTCACAGAGGGAGGTGT
CGCCCGTGTCTCTGAAGGCGCCCGCCCGGGCGACTACGTGGCTCGCGTCTCGGTGTCTGACGCGGGCG
GTGACTGGGAGAAGGAAGATGAGGCCACAGGGGAGCTTGGTGTGACAGCCTCTGATGCAGATTCAGGA
CTCTATGGCTTTATTGAATATTCTCTTTATGATGGATTCCTGAGCTATGAAGCACCTCAGGCATTCCG
GATCGACCCTCATGATGGGCAAATCTGTGTTTCTCAAGATATCGACAGGGAAAGGGATCCAGCTACCT
ATGATCTCCTGGTGGAAGCTAAGGATGGGTTTGAAATCATGCCAGGTGCTTCATTTGAATTATTCGAG
ATAAATTCTGACACTGGAGAGGTAGTGACAACCACCATACTTGACAGAGAAATTCAAGAAGTCTTCAC
CCTTCGAGTACTAGTACGAGATGGGGGATTCCCTTCATTGTCCAGCACCACAACAATCCTCTGCACTG
TTGAAGATGAAAACGATCACGCACCAGAGTTTATTGTTTCCAGTTATGACATTGAGGTTCTGGAAAAC
CAGGAACCAGAGGTTGTCTATACGGTTTTAGCCTCTGATATGGATGCTGGCAATAACAGAGCTGTTGA
ATATCACATAATTGACTCCTCAGAACCAATCTTTTACAGGATTTCTTCTGGTGATCTCGGCGGAAAGT
TCTCCATTCACCCGCGGCTGGGCACTATTCGCACCCGGAAGCCCCTGGATCACGAGACGCAGCCCGTG
GTTGTGCTCACGGTGCAGGCGCAGCTCGGCAGCGCCCCAGCCTGCAGCAGCACCGAGGTCAACATAAC
AGTCATGGATGTCAATGACAACCACCCAGCGTTCCTCAGGACCTCGGATGAGATTAGAATATCCCAGA
CCACGCCCCCTGGCACAGCCTTGTACCTCGCACGTGCGGAAGACAGAGACAGTGGGCGGAACGGACTC
ATCCGGTACTCCATCGCCAGCCCGCAGCCAGGCGTCTTTGCCATCGACAGAGCCCTGGGGGTGCTGTT
CCTCAACGGCAGCCTGGGCGCGGGCGAGCAGCGGGAGCTCACGCTGACTCTCAGGGCCGAGGACCAAG
GCGTGCATCCTCAGGCAGCCCTGCTGGTGCTGACAGTCGTTATCGAGAAACGCGAACACAGCCCATCC
TGGACTTTCGAACATTTGGTCTATCAAGTGGAAGTCAGTACTTCTATTGTGACTGTTAAAGCTTTTGC
TCCTGACTCAATTCAGGACAGCATGAAATATTCAATTTTTAGTGGAAATGAAGATGGAGTTCTTTCCC
TGTGCTCTAAGTCAGGTGTGGTGAACTGCCTTGCTTCTCTCAGTCACACAGACTTTCTCTCCCTGAAA
TTTGAATCTTCGGTGAAGGGACACCAAGACAGAGACAAATTACAGCCAATTCATCTTGATGACAACAA
CTCAAAGAAGCTGTGCTTTACATTCCCTAGAGCCACTCAGGCTCTTGTATTCACTGGGCACTGTCTTT
CTGATACATCTCTCCCCGGTTGGGTTTTTGCTACCGACTTGGACAGTGGTTTGAACGGCCTGATTGAG
TATTCTATTCTGTCTGGCAACCAAGAAGAAGCATTCCAGATTGATGCACTGAGTGGTGTGATAACAAC
AAAAGCGATTCTAGATTACGAGCTCACCAGCTCCTACAGCTTGATTGTCCAAGCCACAGATAAAGGGA
TGCCCAGGCTTTCTAATACGACTGTAATCAAGGTACAGGTGACTGATATAAATGACAATGCCCCAGCT
TTTCTCCCCTCTGAAGCAGTGGAAATTACAGAAGTCATGACTATTTCAGAAGATTCTTTGCCTGGTGT
AATTGTGACTCATGTGTCAGTTCATGATGTGGATTTGAATTCAGCTTTCATATTCAGTTTTGCCAAAG
AGAGTAATCCTGGAACCAAGTTTGCTATTGATCAGAACACTGGAGTGGTGGTGTTGGTGAAAACATTG
GATTTTGAAGAAATGACTGAATATGAGCTGCTCATCCAAATTTCTGATTCAGTGCACTACACAGAGGG
AGCACTTGTAGTCCGTGTGCTGGATGTCAATGATAATCCACCAGTGTTTTCTCAAGATTTCTATCAGG
TCACAGTTCCTGAATCAATACCTGTGGGGTATTCAGTGCTGACTCTGTCAGCCACAGACTTAGAAAGC
AATGAGAACATTTCTTACAGAATTCTATCCTCTTCTAAGGAATTCTCCATTGATCCTAAGAATGGCAC
AATATTTACTATCAGTCCCGTATTACTTCTGGATACAATATCAACAACTCGATTTCTTGTGGAAGCCA
GTGATGGTGGAAATCCTGACCCGAGAGCTCTTACTTTAGTGGAGATAGGAATAGAAGATATGAACAAT
TATGCCCCTGAATTCACAGTCAAATCCTATAATCTTAGCCTAAGTGAGGATGCTCTGGTTGGAAGCAC
GCTTGTTACATTTTCAAACATCGACCATGACTGGACCCGTGAAAACACATATGTTGAATATTCCATCA
TCAGTGGTAATTCACAGAACAATTTTCATGTGGAAACTAAGTTCTTTCATTCAGAATATCCTTATAAG
CAAGTCGGTTATCTTGTGTTGCTTCACAGTCTGGACAGAGAAGCAAGTGCTAGCCATGAGCTTGTCAT
TCTGGCATCTGACAGTGGCTGCCCTCCATTGAGTTCCACAGCTGTCATATCAATACAAGTACTTGATG
TCAATGACAATCCCCCAAACTTCAGCAGCCTGAGCTATCACACCCATGTCAAGGAAAGCACCCCTCTA
GGGAGTCACATCACTGTGGTCTCAGCAAATGACCGTGACACAGGGTCACATGCAGAAATCATCTACAA
CATCATCTCTGGAAATGAGAAGGGACATTTTTACTTAGAAGAAAACACTGGAGTTCTTTATTTGATTA
AACCTCTGGATTATGAAAAAATGACAAAATTCACCTTAACTGTCCAAGCTTCAGATGCAGAAAAGAAA
CATTTTTCTTTTGCAGTTGTGTTTGTCAGTGTCCTGGATGATAACGACCATGCACCTCAGTTTATGTT
CTCAAGCTTCAGCTGTATTGTTCCAGAAAATCTGCCTATTTCCTCTACCATATGCTCTATAAATGCTC
TGGATTTTGATGCTGGTCCGTATGGAGAATTGACCTATTCTATTGTATCACCCTGTTTTCTCACTCAT
GGAATGTCTTATGATCATGATCTCTTCCTCATTGACCCTTTGACAGGGGATATTCATGCTAAGCAAAT
CCTTGACTATGAAAATGGCAATAAATACTGCCTCACAGTCCAAGCCAAAGACAAAGGTGATGCAACTG
CCTCCTTAGTGGTCTGGGTGGATATTGAAGGGATAGATGAATTTGAGCCCATTTTCACTCAAGATCAG
TATTTTTTCACCCTCCCAGAAAAGAATAAAGACAGACAGTTGATTGGCAGAGTGGAAGCCTCAGATGC
AGATGCTGGTATTGATGGAGTCATTCTTTACTCCCTTGGAACCTCATCTCCTTTCTTTTCAGTAAATA
GAACCAATGGAAATATTTATTTGATTAGAGCCCTTCCCCTAATAAAAAGTCAACTCAACAAAGAAGAC
ACCTTGGAAATGAAAATAATCGCTCATAGTCCCAAATCAGATTCCAAGTTTGCATCTTGCACTGTTTT
TGTGAATGTGTCTTTCTCCTCTGAAGGAACACCCTTGGCAGTGTTCGCCAGCAGCTTTTCAATCAGCC
TGGTGGTCTCCTTTTTAGTGTTTCTGATACTCATCTGCATTCTAATTGTAATGATTTTAAGACATAAA
CAAAAAGACACAATAAACAATTATGAGGAGAAGAAAACCTCATCTTTAGATGCGGACTTGAGAGTGAC
CCGGGATGCCAGTGTGCTCAAAGCCTTCCAGAAAACTGACGACTGCAGTAACGAGGTGGTCCCTGTGG
ATGCCACTCCGGAATGGTTGAGTTTAATAAGTATCATGGAGAAGGATATTGTCAATCTGTACAGATAC
TCAAACTCCAGTGGCCACTGTTCTGTGGAAGGAGAAACTGCAGAAGATAAGGAAATCCAGAGGATAAA
TGAGCATCCCTACAGAAAGTGCTCAGACTCAGCTCTGAGTGACCACGAGTCCAGGGTGCCAGACTCGG
GTATCCCGAGGGACTCAGACCAGCTCTCCTGCCTATCTGGGGAAACTGATGTGATGGTGACTGCCGAA
ACAGCAGAAGCCAGCCAAACATTTGGGGAAGGAGATCAAGGGGAAGGCTGCAGCACCACCTGTGCTCA
AAATAATGTGTTACCCCAGACAGTTCAGAAGAGAGAGGCAAAAGAGAGCATCCTGGCTGACGTTAGAA
AAGAGTCTGTCTTTATTTCAGGTGATCAGGAAGTAAGGTGTGCAGCTCTTTCAACTCAGACGACCTCT
GATCATGATGGAAAAGACAACTATCACTGGAATTATCTTCTTAGTTGGGAGCCCAAATTCCAACCTCT
TGCCTCAGTATTTAATGATATTGCAAAACTAAAGGATGAACATTTGCATATGCCTGGCATTCCAAAAG
AGAAGAAATCTTTTGTTTTTCCACCCCCTTTGATAACAGCAGTAGCCCAGCCTGGGATTAAAGCAGTC
CCACCAAGAATGCCGGCAGTAAACCTGGGGCAGGTGCCTCCGAAACACCCACGCTCTCCCATCCCCTA
CCATCTTGGTTCTCTGCCAGAAGGCATGACTCCCAATTTTTCTCCATCTCTTTCCCTATTGACGATGC
AGCCTCCTGCCTTGTCTCCACTGTTGAGAGAAGGAGAATTATTAGGAACACACATCAGTGGTACATGC
CATGAACTTAAAGCAGAAGATGAAGTTCAAATATGAAACCACTGGGATGCCAAGTACCTGCTCACCAT
TGGTCATGAATGAATGAACAAAATGTTTTCAAGCCGGCAACTCGAGATTGGGCTCATTTTTATCTAAA
AGCAAGTGATGTAATTTAGTTAGAGTTTTTAAAACTTCCCCATTAAAGTTTCTCCAATTTCAAAAAAA
AAAAAAAAAAA

| | | | |
|---|---|---|---|
| NOV50h, CG58567-05 | SEQ ID NO: 1080 | 3003 aa | MW at 329184.0kD |
| Protein Sequence | | | |

MEKCGLKEEGSYADIDPVAHDPDAGLFSTQGYTLVQPSDLPKDPAGPFFQLRYRTPGPLPSPLLPGSS
SPLEPLDLVLLRRLDREEAAAHRLQIEAWDGGRPRRTGLLSVELRVLDENDNPPVFEQDEYRAAVRED
AQPGAEVCRVRATDRDLGPNGFVRYSVRARQVPGAGSGGGALGDAAYFAVEELSGVVRVWRPLDREAQ
AWHQLVVEARDGGAEPEVATVRVSIAVLDVNDNRPAIHVLFLTEGGVARVSEGARPGDYVARVSVSDA
DGVIGKITAIDMDSGKNGQLLYFLLSDGKFFKMNPNTGELINWVALDREHRGHHEMTVLVTDRGSPPR
NATMAVYVSVTDINDNRPFFPQCLPGKELHVKVLEGQPVNMLVTTVFAKDPDEGNNAEVTYSVSSEDS
SDHFKIDANNGEIRTTTILSYDYRPSYRMSVIATDQGVPPLQGQAVVNIQELDYETTSHYLFRVITTD
HSKNLSLSSTVFLSIDVEDQNDHSPSFQDELIVISVEENVPIGTLVYVFNAKDDDGSFLNSRIQYYIE
SHNPGTNPFLIHPSFGTLVTVSRLDRESIPTVILTVTASDQAVNVTDRRLRSLTAQIVILDVNDHNPT
FISFPNAHVKEDVTVGSLVHHITAHDPDEGRNGKVTYSILSGNENMTFMLDESSGLLTTTCPLDYEMK
TQHILTVLALDDGTPALSSSQTLTVTVLDVNDEAPVFKQHLYEASVKENQNPGEFVTRVEALDRDSVF
LNTRELNMCFLAFYDAVFKNGGLSAQAFVRVDLEDVNDNHPVFNPSTYVTSISDETQPGTEIINVLAT
DQDSGIYGTVAYELIPGNVSSLFTIDSTTGIIYLTLPLSHLESTTLSLMVSAQDGGGLTAVINADVTI
HIFQTTLAPAEFERPKYTFLVYEDVPEDSPIGTVKAREPLNPPRSSVIHLQVRVLDANDHSPSFPTLY
YQSSVREDAEVGTVVLVLSAVDKDEGLNGQTEYFLTDEACGAFTIDPMSGTLKTSNTLDREARSQHTF
SAVARDCSIQGSRSTTVIIKVYVTDVNDNDPVLEQNPFDVFLSPESPTNQTTVIVRADDLDLGPNGTV
DSDDSPLLDDFHVHPDTGIIRTARRLDRERRDHYSFVAATLLGAVVQVEIRVNDVNDHSPRFPLDSLQ
LDVSELSPPGTAFRLPVAHDPDAGLFSTQGYTLVQPSDLPKDPAGPFFQLRYRTPGPLPSPLLPGSSS
PLEPLDLVLLRRLDREEAAAHRLQIEAWDGGRPRRTGLLSVELRVLDENDNPPVFEQDESRAAVREHA
QPGAEVCRVRATARDVGPNGFVRYSVRARQVPGAGSGGGALGDAAYFALVVEARDGGAEHEVAAVRVS
IAVLDVNDNRPAIHVLFLTEGGVARVSEGARPGDYVARVSVSDAGGDWEKEDEATGELGVTASDADSG
LYGFIEYSLYDGFLSYEAPQAFRIDPHDGQICVSQDIDRERDPATYDLLVEAKDGFEIMPGASFELFE
INSDTGEVVTTTILDREIQEVFTLRVLVRDGGFPSLSSTTTILCTVEDENDHAPEFIVSSYDIEVLEN
QEPEVVYTVLASDMDAGNNRAVEYHIIDSSEPIFYRISSGDLGGKFSIHPRLGTIRTRKPLDHETQPV
VVLTVQAQLGSAPACSSTEVNITVMDVNDNHPAFLRTSDEIRISQTTPPGTALYLARAEDRDSGRNGL
IRYSIASPQPGVFAIDPALGVLFLNGSLGAGEQRELTLTLRAEDQGVHPQAALLVLTVVIEKREHSPS
WTFEHLVYQVEVSTSIVTVKAFAPDSIQDSMKYSIFSGNEDGVLSLCSKSGVVNCLASLSHTDFLSLK
FESSVKGHQDRDKLQPIHLDDNNSKKLCFTFPRATQALVFTGHCLSDTSLPGWVFATDLDSGLNGLIE
YSILSGNQEEAFQIDALSGVITTKAILDYELTSSYSLIVQATDKGMPRLSNTTVIKVQVTDINDNAPA
FLPSEAVEITEVMTISEDSLPGVIVTHVSVHDVDLNSAFIFSFAKESNPGTKFAIDQNTGVVVLVKTL
DFEEMTEYELLIQISDSVHYTEGALVVRVLDVNDNPPVFSQDFYQVTVPESIPVGYSVLTLSATDLES
NENISYRILSSSKEFSIDPKNGTIFTISPVLLLDTISTTRFLVEASDGGNPDPRALTLVEIGIEDMNN
YAPEFTVKSYNLSLSEDALVGSTLVTFSNIDHDWTRENTYVEYSIISGNSQNNFHVETKFFHSEYPYK
QVGYLVLLHSLDREASASHELVILASDSGCPPLSSTAVISIQVLDVNDNPPNFSSLSYHTHVKESTPL
GSHITVVSANDRDTGSHAEIIYNIISGNEKGHFYLEENTGVLYLIKPLDYEKMTKFTLTVQASDAEKK
HFSFAVVFVSVLDDNDHAPQFMFSSFSCIVPENLPISSTICSINALDFDAGPYGELTYSIVSPCFLTH
GMSYDHDLFLIDPLTGDIHAKQILDYENGNKYCLTVQAKDKGDATASLVVWVDIEGIDEFEPIFTQDQ
YFFTLPEKNKDRQLIGRVEASDADAGIDGVILYSLGTSSPFFSVNRTNGNIYLIRALPLIKSQLNKED
TLEMKIIAHSPKSDSKFASCTVFVNVSFSSEGTPLAVFASSFSISLVVSFLVFLILICILIVMILRHK
QKDTINNYEEKKTSSLDADLRVTRDASVLKAFQKTDDCSNEVVPVDATPEWLSLISIMEKDIVNLYRY
SNSSGHCSVEGETAEDKEIQRINEHPYRKCSDSALSDHESRVPDSGIPRDSDQLSCLSGETDVMVTAE
TAEASQTFGEGDQGEGCSTTCAQNNVLPQTVQKREAKESILADVRKESVFISGDQEVRCAALSTQTTS
DHDGKDNYHWNYLLSWEPKFQPLASVFNDIAKLKDEHLHMPGIPKEKKSFVFPPPLITAVAQPGIKAV
PPRMPAVNLGQVPPKHPRSPIPYHLGSLPEGMTPNFSPSLSLLTMQPPALSPLLREGELLGTHISGTC
HELKAEDEVQI

| | | |
|---|---|---|
| NOV50i, CG58567-06 | SEQ ID NO: 1081 | 10267 bp |
| DNA Sequence | ORF Start: ATG at 1 | ORF Stop: TGA at 10084 |

ATGGAGAAATGTGGGCTTAAAGAGGAAGGCAGTTACGCGGACATAGATCCAGTTGCCCACGATCCGGA
CGCCGGACTGTTCAGCACTCAGGGCTACACCCTGGTGCAACCGTCCGACCTGCCCAAGGACCCCGCAG
GCCCGTTCTTCCAGTTGCGCTACCGGACTCCGGGGCCACTACCGTCACCGCTTTTGCCAGGCTCCTCG
TCACCCCTGGAGCCTCTAGATCTGGTGCTGCTGCGGCGCTTGGACCGAGAGGAGGCGGCGGCGCACCG
GCTGCAGATCGAGGCATGGGACGGCGGCCGACCCCGGCGCACCGGCCTCCTGAGCGTGGAGCTGCGCG
TGCTGGATGAGAACGACAACCCGCCGGTCTTTGAGCAGGACGAGTACCGCGCCGCGGTGCGCGAGGAC
GCCCAGCCGGGCGCCGAGGTCTGTCGCGTGCGCGCCACCGACCGCGACCTGGGGCCCAATGGCTTCGT
GCGCTACAGCGTCCGCGCCCGGCAAGTGCCTGGGGCGGGTAGCGGCGGCGGGGCACTGGGCGACGCGG
CCTACTTCGCGGTGGAGGAGCTGAGCGGCGTGGTGCGAGTGTGGAGACCTCTGGACCGCGAGGCACAG
GCCTGGCACCAGTTGGTGGTGGAGGCCCGCGATGGAGGCGCCGAGCCTGAGGTTGCCACGGTGCGCGT
GTCCATCGCCGTGCTGGACGTGAATGACAACCGGCCAGCAATTCACGTGCTCTTTCTCACAGAGGGAG
GCGTCGCCCGTGTCTCTGAAGGCGCCCGACCGGGCGACTACGTGGCTCGCGTCTCGGTGTCTGACGCG
GACGGTGACTGGGAGAAGGAAGATGAGGCCACAGGGGAGCTTGGTGTGGGTCTTGGAGACGGGAGCAT
CTCTCTGTCCTTGGAAGGCGGAGAGGGAGACTTCGCGTTGCTACCCGGCGGCCCCCCAGGGGTATTTT
TCCTTTGCGTGGAGGGGCCCCTGGACAGAGAGAGCCGCGATCTGTATGAGTTACTACTGGTGGCCACG
GACGCGGGGTCCCCGCCGCTGAGCACGGAGGAGACGCTGCTACTCCGGGTCGCTGACCTCAATGACCA
ACCACCTCTCTTCAGCCAACAGCATTACAAGGCCTCAGTGTCCGAGGCCGCGGCCCCTGGCACTGTAG
TCATGTGGGTCAGCGCCTCCGATGCCGACGAGGCAGGCAGTGATCACGCCTGGCTGCGCTACACTGTA
GTCCAACTCTCGGCTCCCTGCAATCTCGGCTCCCTGCAATCAAAGATGGTCCACACCGCAGAGTGTGG
ACCATCTTTTGCCATTGATTCCGAAAGCGGTGCGATCAGCACTATCCGGACTCTAGACCGAGAGGTCC
AGGAGGCGGTGGAGCTGAAAGTGGTGGCCCAGGACCTCGGAGAGCCCCCACTCTCTGCCACCTGCCTG
GTGAGCATCACCGTAGATGATGTGAATGACAATGAGCCCATCTTCTGGAGGCAGGTGTACAATGCCAC
CATTGCAGAGCATGCCCCGGTTGGACACTGCTTTCTGCAGCTTATATCTGCTCAAGTTGCCTCTGTCA
AAATCAAACACAAACACAAGGAGATACACGAGAAACACAATCTTGCCTATATTTCCTGTCCAGCAGGC
ACCATCTATGTCATAACCTGGGCAGATGGTGCTGCTGCCTTTAGTGGGACAGACTTTGCATTCAGTTC
TGATGAACTTCAAGCCTTTGTTCTCAAGTCTCTGTTCTGTGAATTAGGAGAAGGAGAGTTAATCAATT
GGGTGGCACTGGATCGTGAGCACCGGGGGCACCATGAGATGACTGTGCTAGTGACAGACCGCGGCTCC
CCACCACGAAACGCCACCATGGCGGTTTACGTCTCAGTTACTGACATCAATGATAACAGGCCCTTCTT
CCCCCAGTGTCTCCCTGGAAAGGAGTTACACGTGAAGGTTCTGGAAGGTCAACCAGTAAATATGTTGG
TTACAACTGTGTTTGCAAAGGATCCTGATGAAGGAAATAATGCAGAAGTTACATACTCAGTATCTTCA
GAAGATAGTTCTGATCACTTTAAGATTGACGCCAACAATGGTGAAATAAGAACAACCACAATACTTTC
GTATGATTATAGACCTTCCTACAGAATGAGTGTCATTGCCACTGACCAGGGAGTGCCTCCTCTTCAAG
GACAGGCAGTTGTTAATATTCAGGTGATCCCACTATCCAAAGGGAGAGCAATCATGTCTCAGAATATT
AGACATTTAATTATACCAGAAAATTTGAAGCCCACAAAAATAATGAGCTTGATAAAGTCATCTGATCA
CCTTCAACAACATTATAATGGAAAGTTACATTTTAGTATTGTTGCAGATGATAAGGATGGACACTTTG
AAATAGACAGCTCAACCGGAGACTTGTTTCTTTCTAAGGAACTTGATTATGAGACGACATCTCATTAT
CTTTTCAGAGTGATTACTACAGACCATAGCAAAAACCTTTCCCTGAGTAGCACAGTCTTCCTTAGTAT
CGATGTGGAAGATCAGAATGACCATTCCCCATCTTTCCAGGATGAGCTCATTGTGATCAGTGTAGAGG
AGAATGTTCCCATAGGAACCCTGGTGTATGTCTTCAATGCCAAAGATGATGACGGCAGTTTTTTGAAC
AGTAGAATACAATACTACATTGAATCCCACAACCCTGGCACGAATCCATTTCTCATCCACCCCTCATT
TGGCACACTAGTCACTGTGTCCCGTCTTGACAGAGAAAGCATTCCAACTGTCATCCTGACAGTAACAG
GATGTAAATGACCACAACCCCACTTTTATTTCTTTCCCCAATGCCCATGTCAAAGAGGATGTCACAGT
GGGCTCCTTGGTCCACCACATAACTGCTCACGATCCAGACGAAGGAAGGAATGGAAAAGTAACATACA
GCATCCTCTCAGGAAATGAAAACATGACGTTTATGCTAGATGAGTCATCAGGCTTACTAACCACAACC
TGTCCTTTGGATTATGAAATGAAAACTCAGCATATTCTGACTGTTCTGGCACTGGATGATGGCACACC
AGCACTTTCTTCATCCCAGACTTTGACAGTTACTGTTCTTGATGTAAATGATGAAGCTCCAGTATTTA
AGCAGCACCTGTATGAAGCCTCAGTGAAAGAAAACCAAAATCCAGGGGAGTTTGTTACCAGGGTTGAA
GCTCTGGACAGAGATTCAGGTATGAGGCTGAATGGAGATCCAGACAGGGAGCTGTGTGCAGGAGGGAA
CCCTCTTGGAAGCAGGGCCCCTCCTGGAAGCAGGACCCCTCCTGAAGGTGGGCTAAGTGCCCAAGCCT
TTGTTCGTGTGGACCTGGAGGACGTGAATGATAATCATCCTGTGTTTAACCCATCAACCTATGTGACG
AGCATCAGTGATGAGACCCAGCCAGGCACCGAGATCATCAATGTTCTTGCCACTGACCAGGACTCTGG
GATATATGGGACAGTGGCTTATGAGCTTATTCCAGGAAACGTGTCGTCCCTTTTTACCATTGACTCCA
CCACAGGAATTATTTACTTAACATTACCTCTTAGTCATTTGGAATCTACCACACTTTCGTTGATGGTC
TCTGCTCAAGACGGTGGTGGGCTCACAGCTGTCATTAATGCCGATGTCACCATACACATTTTCCAGAC
AACTCTGGCACCTGCTGAGTTTGAAAGGCCTAAGTACACTTTCTTAGTTTATGAAGATGTGCCTGAAG
ATAGTCCCATTGGAACAGTGAAAGCAAGAGAGCCCTTGAATCCACCTAGGAGCTCTGTAATACACCTG
CAAGTTAGAGTTTTGGATGCCAATGACCACAGTCCTTCTTTTCCCACACTTTATTACCAGTCCTCTGT
GAGAGAAGATGCTGAAGTGGGAACAGTGGTTCTTGTGCTTTCAGCTGTGGACAAGGATGAAGGCCTGA
ATGGGCAAACTGAGTATTTTCTGACTGATGAGGCTTGTGGTGCATTCACCATTGATCCTATGTCAGGC
ACATTAGAAACCAGCAACACCCTCGACCGTGAAGCCAGATCTCAGCATACATTTAGTGCTGTGGCCAG
AGACTGTAGCATCCAGGGTTCACGAAGCACCACTGTAATTATAAAAGTATATGTCACTGATGTTAATG
ACAATGATCCAGTTTTGGAACAGAACCCTTTTGATGTGTTTCTTTCCCCCGAGTCGCCTACAAACCAG
ACAACTGTCATTGTGAGAGCTGATGACCTGGACTTGGGGCCCAATGGAACTGTGGACTCCGATGACTC
CCCGCTGCTGGACGACTTCCACGTGCACCCGGACACCGGCATCATCCGCACTGCGCGGCGCCTGGACC
GCGAGCGGCGGGACCACTACAGCTTCGTCGCCGCCACGCTGCTGGGCGCTGTGGTGCAGGTGGAGATT
CGCGTCAACGACGTGAATGACCACTCGCCCCGCTTTCCCCTCGACTCCCTGCAACTCGACGTCTCCGA
GCTCAGCCCGCCAGGGACCGCCTTCCGCCTGCCAGTTGCCCACGATCCGGACGCCGGACTGTTCAGCA
CTCAGGGCTACACCCTGGTGCAACCGTCCGACCTGCCCAAGGACCCCGCAGGCCCGTTCCCCCAGTTG
CGCTACCGGACTCCGGGGCCACTACCGTCACCGCTTTTGCCAGGCTCCTCGTCACCCCTGGAGCCTCT
AGATCTGGTGCTGCTGCGGCGCTTGGACCGAGAGGAGGCGGCGGCGCACCGGCTGCAGATCGAGGCAT
GGGACGGCGGCCGACCCCGGCGCACCGGCCTCCTGAGCGTGGAGCTGCGCGTGCTGGATGAGAACGAC
AACCCGCCGGTCTTTGAGCAGGACGAGTCCCGCGCCGCGGTGCGAGAGCACGCCCAGCCGGGCGCCGA
GGTCTGTCGCGTGCGCGCCACCGCCCGCGACGTGGGGCCCAATGGCTTCGTGCGCTACAGCGTCCGCG
CCCGGCAAGTGCCTGGGGCGGGTAGCGGCGGCGGGGCACTGGGCGACGCGGCCTACTTCGCGTTGGTG
GTGGAGGCCCGCGATGGAGGCGCCGAGCATGAGGTTGCCGCGGTGCGTGTGTCCATCGCCGTGCTGGA
CGTGAATGACAACCGGCCAGCAATTCACGTGCTCTTTCTCACAGAGGGAGGTGTCGCCCGTGTCTCTG
AAGGCGCCCGCCCGGGCGACTACGTGGCTCGCGTCTCGGTGTCTGACGCGGGCGGTGACTGGGAGAAG
GAAGATGAGGCCACAGGGGAGCTTGGTGTGACAGCCTCTGATGCAGATTCAGGACTCTATGGCTTTAT
TGAATATTCTCTTTATGATGGATTCCTGAGCTATGAAGCACCTCAGGCATTCCGGATCGACCCTCATG
ATGGGCAAATCTGTGTTTCTCAAGATATCGACAGGGAAAGGGATCCAGCTACCTATGATCTCCTGGTG
GAAGCTAAGGATGGGTTTGAAATCATGCCAGGTGCTTCATTTGAATTATTCGAGATAAATTCTGACAC
TGGAGAGGTAGTGACAACCACCATACTTGACAGAGAAATTCAAGAAGTCTTCACCCTTCGAGTACTAG
TACGAGATGGGGGATTCCCTTCATTGTCCAGCACCACAACAATCCTCTGCACTGTTGAAGATGAAAAC
GATCACGCACCAGAGTTTATTGTTTCCAGTTATGACATTGAGGTTCTGGAAAACCAGGAACCAGAGGT
TGTCTATACGGTTTTAGCCTCTGATATGGATGCTGGCAATAACAGAGCTGTTGAATATCACATAATTG
ACTCCTCAGAACCAATCTTTTACAGGATTTCTTCTGGTGATCTCGGCGGAAAGTTCTCCATTCACCCG
CGGCTGGGCACTATTCGCACCCGGAAGCCCCTGGATCACGAGACGCAGCCCGTGGTTGTGCTCACGGT
GCAGGCGCAGCTCGGCAGCGCCCCAGCCTGCAGCAGCACCGAGGTCAACATAACAGTCATGGATGTCA
ATGACAACCACCCAGCGTTCCTCAGGACCTCGGATGAGATTAGAATATCCCAGACCACGCCCCCTGGC
ACAGCCTTGTACCTCGCACGTGCGGAAGACAGAGACAGTGGGCGGAACGGACTCATCCGGTACTCCAT
CGCCAGCCCGCAGCCAGGCGTCTTTGCCATCGACAGAGCCCTGGGGGTGCTGTTCCTCAACGGCAGCC
TGGGCGCGGGCGAGCAGCGGGAGCTCACGCTGACTCTCAGGGCCGAGGACCAAGGCGTGCATCCTCAG
GCAGCCCTGCTGGTGCTGACAGTCGTTATCGAGAAACGCGAACACAGCCCATCCTGGACTTTCGAACA
TTTGGTCTATCAAGTGGAAGTCAGTACTTCTATTGTGACTGTTAAAGCTTTTGCTCCTGACTCAATTC
AGGACAGCATGAAATATTCAATTTTTAGTGGAAATGAAGATGGAGTTCTTTCCCTGTGCTCTAAGTCA
GGTGTGGTGAACTGCCTTGCTTCTCTCAGTCACACAGACTTTCTCTCCCTGAAATTTGAATCTTCGGT
GAAGGGACACCAAGACAGAGACAAATTACAGCCAATTCATCTTGATGACAACAACTCAAAGAAGCTGT
GCTTTACATTCCCTAGAGCCACTCAGGCTCTTGTATTCACTGGGCACTGTCTTTCTGATACATCTCTC
CCCGGTTGGGTTTTTGCTACCGACTTGGACAGTGGTTTGAACGGCCTGATTGAGTATTCTATTCTGTC
TGGCAACCAAGAAGAAGCATTCCAGATTGATGCACTGAGTGGTGTGATAACAACAAAAGCGATTCTAG
ATTACGAGCTCACCAGCTCCTACAGCTTGATTGTCCAAGCCACAGATAAAGGGATGCCCAGGCTTTCT
AATACGACTGTAATCAAGGTACAGGTGACTGATATAAATGACAATGCCCCAGCTTTTCTCCCCTCTGA
AGCAGTGGAAATTACAGAAGTCATGACTATTTCAGAAGATTCTTTGCCTGGTGTAATTGTGACTCATG
TGTCAGTTCATGATGTGGATTTGAATTCAGCTTTCATATTCAGTTTTGCCAAAGAGAGTAATCCTGGA
ACCAAGTTTGCTATTGATCAGAACACTGGAGTGGTGGTGTTGGTGAAAACATTGGATTTTGAAGAAAT
GACTGAATATGAGCTGCTCATCCAAATTTCTGATTCAGTGCACTACACAGAGGGAGCACTTGTAGTCC
GTGTGCTGGATGTCAATGATAATCCACCAGTGTTTTCTCAAGATTTCTATCAGGTCACAGTTCCTGAA
TCAATACCTGTGGGGTATTCAGTGCTGACTCTGTCAGCCACAGACTTAGAAAGCAATGAGAACATTTC
TTACAGAATTCTATCCTCTTCTAAGGAATTCTCCATTGATCCTAAGAATGGCACAATATTTACTATCA
GTCCCGTATTACTTCTGGATACAATATCAACAACTCGATTTCTTGTGGAAGCCAGTGATGGTGGAAAT
CCTGACCCGAGAGCTCTTACTTTAGTGGAGATAGGAATAGAAGATATGAACAATTATGCCCCTGAATT
CACAGTCAAATCCTATAATCTTAGCCTAAGTGAGGATGCTCTGGTTGGAAGCACGCTTGTTACATTTT
CAAACATCGACCATGACTGGACCCGTGAAAACACATATGTTGAATATTCCATCATCAGTGGTAATTCA
CAGAACAATTTTCATGTGGAAACTAAGTTCTTTCATTCAGAATATCCTTATAAGCAAGTCGGTTATCT
TGTGTTGCTTCACAGTCTGGACAGAGAAGCAAGTGCTAGCCATGAGCTTGTCATTCTGGCATCTGACA
GTGGCTGCCCTCCATTGAGTTCCACAGCTGTCATATCAATACAAGTACTTGATGTCAATGACAATCCC
CCAAACTTCAGCAGCCTGAGCTATCACACCCATGTCAAGGAAAGCACCCCTCTAGGGAGTCACATCAC
TGTGGTCTCAGCAAATGACCGTGACACAGGGTCACATGCAGAAATCATCTACAACATCATCTCTGGAA
ATGAGAAGGGACATTTTTACTTAGAAGAAAACACTGGAGTTCTTTATTTGATTAAACCTCTGGATTAT
GAAAAAATGACAAAATTCACCTTAACTGTCCAAGCTTCAGATGCAGAAAAGAAACATTTTTCTTTTGC
AGTTGTGTTTGTCAGTGTCCTGGATGATAACGACCATGCACCTCAGTTTATGTTCTCAAGCTTCAGCT
GTATTGTTCCAGAAAATCTGCCTATTTCCTCTACCATATGCTCTATAAATGCTCTGGATTTTGATGCT
GGTCCGTATGGAGAATTGACCTATTCTATTGTATCACCCTGTTTTCTCACTCATGGAATGTCTTATGA
TCATGATCTCTTCCTCATTGACCCTTTGACAGGGGATATTCATGCTAAGCAAATCCTTGACTATGAAA
ATGGCAATAAATACTGCCTCACAGTCCAAGCCAAAGACAAAGGTGATGCAACTGCCTCCTTAGTGGTC
TGGGTGGATATTGAAGGGATAGATGAATTTGAGCCCATTTTCACTCAAGATCAGTATTTTTTCACCCT
CCCAGAAAAGAATAAAGACAGACAGTTGATTGGCAGAGTGGAAGCCTCAGATGCAGATGCTGGTATTG
ATGGAGTCATTCTTTACTCCCTTGGAACCTCATCTCCTTTCTTTTCAGTAAATAGAACCAATGGAAAT
ATTTATTTGATTAGAGCCCTTCCCCTAATAAAAAGTCAACTCAACAAAGAAGACACCTTGGAAATGAA
AATAATCGCTCATAGTCCCAAATCAGATTCCAAGTTTGCATCTTGCACTGTTTTTGTGAATGTGTCTT
TCTCCTCTGAAGGAACACCCTTGGCAGTGTTCGCCAGCAGCTTTTCAATCAGCCTGGTGGTCTCCTTT
TTAGTGTTTCTGATACTCATCTGCATTCTAATTGTAATGATTTTAAGACATAAACAAAAAGACACAAT
AAACAATTATGAGGAGAAGAAAACCTCATCTTTAGATGCGGACTTGAGAGTGACCCGGGATGCCAGTG
TGCTCAAAGCCTTCCAGAAAACTGACGACTGCAGTAACGAGGTGGTCCCTGTGGATGCCACTCCGGAA
TGGTTGAGTTTAATAAGTATCATGGAGAAGGATATTGTCAATCTGTACAGATACTCAAACTCCAGTGG
CCACTGTTCTGTGGAAGGAGAAACTGCAGAAGATAAGGAAATCCAGAGGATAAATGAGCATCCCTACA
GAAAGTGCTCAGACTCAGCTCTGAGTGACCACGAGTCCAGGGTGCCAGACTCGGGTATCCCGAGGGAC
TCAGACCAGCTCTCCTGCCTATCTGGGGAAACTGATGTGATGGTGACTGCCGAAACAGCAGAAGCCAG
CCAAACATTTGGGGAAGGAGATCAAGGGGAAGGCTGCAGCACCACCTGTGCTCAAAATAATGTGTTAC
CCCAGACAGTTCAGAAGAGAGAGGCAAAAGAGAGCATCCTGGCTGACGTTAGAAAAGAGTCTGTCTTT
ATTTCAGGTGATCAGGAAGTAAGGTGTGCAGCTCTTTCAACTCAGACGACCTCTGATCATGATGGAAA
AGACAACTATCACTGGAATTATCTTCTTAGTTGGGAGCCCAAATTCCAACCTCTTGCCTCAGTATTTA
ATGATATTGCAAAACTAAAGGATGAACATTTGCATATGCCTGGCATTCCAAAAGAGAAGAAATCTTTT
GTTTTTCCACCCCCTTTGATAACAGCAGTAGCCCAGCCTGGGATTAAAGCAGTCCCACCAAGAATGCC
GGCAGTAAACCTGGGGCAGGTGCCTCCGAAACACCCACGCTCTCCCATCCCCTACCATCTTGGTTCTC
TGCCAGAAGGCATGACTCCCAATTTTTCTCCATCTCTTTCCCTATTGACGATGCAGCCTCCTGCCTTG
TCTCCACTGTTGAGAGAAGGAGAATTATTAGGAACACACATCAGTGGTACATGCCATGAACTTAAAGC
AGAAGATGAAGTTCAAATATGAAACCACTGGGATGCCAAGTACCTGCTCACCATTGGTCATGAATGAA
TGAACAAAATGTTTTCAAGCCGGCAACTCGAGATTGGGCTCATTTTTATCTAAAAGCAAGTGATGTAA
TTTAGTTAGAGTTTTTAAAACTTCCCCATTAAAGTTTCTCCAATTTCAAAAAAAAAAAAAAAAAAAA

| | | | |
|---|---|---|---|
| NOV50i, CG58567-06 | SEQ ID NO: 1082 | 3361 aa | MW at 367309.1kD |
| Protein Sequence | | | |

MEKCGLKEEGSYADIDPVAHDPDAGLFSTQGYTLVQPSDLPKDPAGPFFQLRYRTPGPLPSPLLPGSS
SPLEPLDLVLLRRLDREEAAAHRLQIEAWDGGRPRRTGLLSVELRVLDENDNPPVFEQDEYRAAVRED
AQPGAEVCRVPATDRDLGPNGFVRYSVRARQVPGAGSGGGALGDAAYFAVEELSGVVRVWRPLDREAQ
AWHQLVVEARDGGAEPEVATVRVSIAVLDVNDNRPAIHVLFLTEGGVARVSEGARPGDYVARVSVSDA
DGDWEKEDEATGELGVGLGDGSISLSLEGGEGDFALLPGGPPGVFFLCVEGPLDRESRDLYELLLVAT
DAGSPPLSTEETLLLRVADLNDQPPLFSQQHYKASVSEAAAPGTVVMWVSASDADEAGSDHAWLRYTV
VQLSAPCNLGSLQSKMVHTAECGPSFAIDSESGAISTIRTLDREVQEAVELKVVAQDLGEPPLSATCL
VSITVDDVNDNEPIFWRQVYNATIAEHAPVGHCFLQLISAQVASVKIKHKHKEIHEKHNLAYISCPAG
TIYVITWADGAAAFSGTDFAFSSDELQAFVLKSLFCELGEGELINWVALDREHRGHHEMTVLVTDRGS
PPRNATMAVYVSVTDINDNRPFFPQCLPGKELHVKVLEGQPVNMLVTTVFAKDPDEGNNAEVTYSVSS
EDSSDHFKIDANNGEIRTTTILSYDYRPSYRMSVIATDQGVPPLQGQAVVNIQVIPLSKGRAIMSQNI
RHLIIPENLKPTKIMSLIKSSDHLQQHYNGKLHFSIVADDKDGHFEIDSSTGDLFLSKELDYETTSHY
LFRVITTDHSKNLSLSSTVFLSIDVEDQNDHSPSFQDELIVISVEENVPIGTLVYVFNAKDDDGSFLN
SRIQYYIESHNPGTNPFLIHPSFGTLVTVSRLDRESIPTVILTVTASDQAVNVTDRRLRSLTAQIVIL
DVNDHNPTFISFPNAHVKEDVTVGSLVHHITAHDPDEGRNGKVTYSILSGNENMTFMLDESSGLLTTT
CPLDYEMKTQHILTVLALDDGTPALSSSQTLTVTVLDVNDEAPVFKQHLYEASVKENQNPGEFVTRVE
ALDRDSGMRLNGDPDRELCAGGNPLGSRAPPGSRTPPEGGLSAQAFVRVDLEDVNDNHPVFNPSTYVT
SISDETQPGTEIINVLATDQDSGIYGTVAYELIPGNVSSLFTIDSTTGIIYLTLPLSHLESTTLSLMV
SAQDGGGLTAVINADVTIHIFQTTLAPAEFERPKYTFLVYEDVPEDSPIGTVKAREPLNPPRSSVIHL
QVRVLDANDHSPSFPTLYYQSSVREDAEVGTVVLVLSAVDKDEGLNGQTEYFLTDEACGAFTIDPMSG
TLKTSNTLDREARSQHTFSAVARDCSIQGSRSTTVIIKVYVTDVNDNDPVLEQNPFDVFLSPESPTNQ
TTVIVRADDLDLGPNGTVDSDDSPLLDDFHVHPDTGIIRTARRLDRERRDHYSFVAATLLGAVVQVEI
RVNDVNDHSPRFPLDSLQLDVSELSPPGTAFRLPVAHDPDAGLFSTQGYTLVQPSDLPKDPAGPFFQL
RYRTPGPLPSPLLPGSSSPLEPLDLVLLRRLDREEAAAHRLQIEAWDGGRPRRTGLLSVELRVLDEND
NPPVFEQDESRAAVREHAQPGAEVCRVRATARDVGPNGFVRYSVRARQVPGAGSGGGALGDAAYFALV
VEARDGGAEHEVAAVRVSIAVLDVNDNRPAIHVLFLTEGGVARVSEGARPGDYVARVSVSDAGGDWEK
EDEATGELGVTASDADSGLYGFIEYSLYDGFLSYEAPQAFRIDPHDGQICVSQDIDRERDPATYDLLV
EAKDGFEIMPGASFELFEINSDTGEVVTTTILDREIQEVFTLRVLVRDGGFPSLSSTTTILCTVEDEN
DHAPEFIVSSYDTEVLENQEPEVVYTVLASDMDAGNNRAVEYHIIDSSEPIFYRISSGDLGGKFSTHP
RLGTIRTRKPLDHETQPVVVLTVQAQLGSAPACSSTEVNITVMDVNDNHPAFLRTSDEIRISQTTPPG
TALYLARAEDRDSGRNGLIRYSIASPQPGVFAIDRALGVLFLNGSLGAGEQRELTLTLRAEDQGVHPQ
AALLVLTVVIEKREHSPSWTFEHLVYQVEVSTSIVTVKAFAPDSIQDSMKYSIFSGNEDGVLSLCSKS
GVVNCLASLSHTDFLSLKFESSVKGHQDRDKLQPIHLDDNNSKKLCFTFPRATQALVFTGHCLSDTSL
PGWVFATDLDSGLNGLIEYSILSGNQEEAFQIDALSGVITTKAILDYELTSSYSLIVQATDKGMPRLS
NTTVIKVQVTDINDNAPAFLPSEAVEITEVMTISEDSLPGVIVTHVSVHDVDLNSAFIFSFAKESNPG
TKFAIDQNTGVVVLVKTLDFEEMTEYELLIQISDSVHYTEGALVVRVLDVNDNPPVFSQDFYQVTVPE
SIPVGYSVLTLSATDLESNENISYRILSSSKEFSIDPKNGTIFTISPVLLLDTISTTRFLVEASDGGN
PDPRALTLVEIGIEDMNNYAPEFTVKSYNLSLSEDALVGSTLVTFSNIDHDWTRENTYVEYSIISGNS
QNNFHVETKFFHSEYPYKQVGYLVLLHSLDREASASHELVILASDSGCPPLSSTAVISIQVLDVNDNP
PNFSSLSYHTHVKESTPLGSHITVVSANDRDTGSHAEIIYNIISGNEKGHFYLEENTGVLYLIKPLDY
EKMTKFTLTVQASDAEKKHFSFAVVFVSVLDDNDHAPQFMFSSFSCIVPENLPISSTICSINALDFDA
GPYGELTYSIVSPCFLTHGMSYDHDLFLIDPLTGDIHAKQILDYENGNKYCLTVQAKDKGDATASLVV
WVDIEGIDEFEPIFTQDQYFFTLPEKNKDRQLIGRVEASDADAGIDGVILYSLGTSSPFFSVNRTNGN
IYLIRALPLIKSQLNKEDTLEMKIIAHSPKSDSKFASCTVFVNVSFSSEGTPIAVFASSFSISLVVSF
LVFLILICILIVMILRHKQKDTINNYEEKKTSSLDADLRVTRDASVLKAFQKTDDCSNEVVPVDATPE
WLSLISIMEKDIVNLYRYSNSSGHCSVEGETAEDKEIQRINEHPYRKCSDSALSDHESRVPDSGIPRD
SDQLSCLSGETDVMVTAETAEASQTFGEGDQGEGCSTTCAQNNVLPQTVQKREAKESILADVRKESVF
ISGDQEVRCAALSTQTTSDHDGKDNYHWNYLLSWEPKFQPLASVFNDIAKLKDEHLHMPGIPKEKKSF
VFPPPLITAVAQPGIKAVPPRMPAVNLGQVPPKHPRSPIPYHLGSLPEGMTPNFSPSLSLLTMQPPAL
SPLLREGELLGTHISGTCHELKAEDEVQI

A ClustalW comparison of the above protein sequences yields the following sequence alignment shown in Table 50B.

TABLE 50B
Comparison of the NOV50 protein sequences.

NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h MEKCGLKEEGSYADIDPVAHDPDAGLFSTQGYTLVQPSDLPKDPAGPFFQLRYRTPGPLP
NOV50i MEKCGLKEEGSYADIDPVAHDPDAGLFSTQGYTLVQPSDLPKDPAGPFFQLRYRTPGPLP
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h SPLLPGSSSPLEPLDLVLLRRLDREEAAAHRLQIEAWDGGRPRRTGLLSVELRVLDENDN
NOV50i SPLLPGSSSPLEPLDLVLLRRLDREEAAAHRLQIEAWDGGRPRRTGLLSVELRVLDENDN
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h PPVFEQDEYRAAVREDAQPGAEVCRVRATDRDLGPNGFVRYSVRARQVPGAGSGGGALGD
NOV50i PPVFEQDEYRAAVREDAQPGAEVCRVRATDRDLGPNGFVRYSVRARQVPGAGSGGGALGD
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h AAYFAVEELSGVVRVWRPLDREAQAWHQLVVEARDGGAEPEVATVRVSIAVLDVNDNRPA
NOV50i AAYFAVEELSGVVRVWRPLDREAQAWHQLVVEARDGGAEPEVATVRVSIAVLDVNDNRPA
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h IHVLFLTEGGVARVSEGARPGDYVARVSVSDADGVIGKITAIDMDS--------------
NOV50i IHVLFLTEGGVARVSEGARPGDYVARVSVSDADGDWEKEDEATGELGVGLGDGSISLSLE
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h ------------------------------------------------------------
NOV50i GGEGDFALLPGGPPGVFFLCVEGPLDRESRDLYELLLVATDAGSPPLSTEETLLLRVADL
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h ---------------------------------------------------------GK-
NOV50i NDQPPLFSQQHYKASVSEAAAPGTVVMWVSASDADEAGSDHAWLRYTVVQLSAPCNLGSL
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h ------------------------------------------------------------
NOV50i QSKMVHTAECGPSFAIDSESGAISTIRTLDREVQEAVELKVVAQDLGEPPLSATCLVSIT
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h --------------------------------NG-QLLYFLLSD----------------
NOV50i VDDVNDNEPIFWRQVYNATIAEHAPVGHCFLQLISAQVASVKIKHKHKEIHEKHNLAYIS
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h ----------G------------------------KFFKMNPNTGELINWVALDREHRGH
NOV50i CPAGTIYVITWADGAAAFSGTDFAFSSDELQAFVLKSLFCELGEGELINWVALDREHRGH
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h HEMTVLVTDRGSPPRNATMAVYVSVTDINDNRPFFPQCLPGKELHVKVLEGQPVNMLVTT
NOV50i HEMTVLVTDRGSPPRNATMAVYVSVTDINDNRPFFPQCLPGKELHVKVLEGQPVNMLVTT
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h VFAKDPDEGNNAEVTYSVSSEDSSDHFKIDANNGEIRTTTILSYDYRPSYRMSVIATDQG
NOV50i VFAKDPDEGNNAEVTYSVSSEDSSDHFKIDANNGEIRTTTILSYDYRPSYRMSVIATDQG
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h VPPLQGQAVVNIQEL---------------------------------------------
NOV50i VPPLQGQAVVNIQVIPLSKGRAIMSQNIRHLIIPENLKPTKIMSLIKSSDHLQQHYNGKL
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h --------D--------------------YETTSHYLFRVITTDHSKNLSLSSTVFLSID
NOV50i HFSIVADDKDGHFEIDSSTGDLFLSKELDYETTSHYLFRVITTDHSKNLSLSSTVFLSID
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h VEDQNDHSPSFQDELIVISVEENVPIGTLVYVFNAKDDDGSFLNSRIQYYIESHNPGTNP
NOV50i VEDQNDHSPSFQDELIVISVEENVPIGTLVYVFNAKDDDGSFLNSRIQYYIESHNPGTNP
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h FLIHPSFGTLVTVSRLDRESIPTVILTVTASDQAVNVTDRRLRSLTAQIVILDVNDHNPT
NOV50i FLIHPSFGTLVTVSRLDRESIPTVILTVTASDQAVNVTDRRLRSLTAQIVILDVNDHNPT
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h FISFPNAHVKEDVTVGSLVHHITAHDPDEGRNGKVTYSILSGNENMTFMLDESSGLLTTT
NOV50i FISFPNAHVKEDVTVGSLVHHITAHDPDEGRNGKVTYSILSGNENMTFMLDESSGLLTTT
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h CPLDYEMKTQHILTVLALDDGTPALSSSQTLTVTVLDVNDEAPVFKQHLYEASVKENQNP
NOV50i CPLDYEMKTQHILTVTALDDGTPALSSSQTLTVTVLDVNDEAPVFKQHLYEASVKENQNP
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h GEFVTRVEALDRDSVFLNTRELNMCFLAFYDAVFK----------NGGLSAQAFVRVDLE
NOV50i GEFVTRVEALDRDSGNRLNGDPDRELCAGGNPLGSRAPPGSRTPPEGGLSAQAFVRVDLE
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h DVNDNHPVFNPSTYVTSISDETQPGTEIINVLATDQDSGTYGTVAYELIPGNVSSLFTID
NOV50i DVNDNHPVFNPSTYVTSISDETQPGTEIINVLATDQDSGTYGTVAYELIPGNVSSLFTID
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h STTGIIYLTLPLSHLESTTLSLNVSAQDGGGLTAVINADVTIHIFQTTLAPAEFERPKYT
NOV50i STTGIIYLTLPLSHLESTTLSLNVSAQDGGGLTAVINADVTIHIFQTTLAPAEFERPKYT
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h FLVYEDVPEDSPIGTVKAREPLNPPRSSVIHLQVRVLDANDHSPSFPTLYYQSSVREDAE
NOV50i FLVYEDVPEDSPIGTVKAREPLNPPRSSVIHLQVRVLDANDHSPSFPTLYYQSSVREDAE
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h VGTVVLVLSAVDKDEGLNGQTEYFLTDEACGAFTTDPMSGTLKTSNTLDREARSQHTFSA
NOV50i VGTVVLVLSAVDKDEGLNGQTEYFLTDEACGAFTIDPMSGTLKTSNTLDREARSQHTFSA
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h VARDCSIQGSRSTTVIIKVYVTDVNDNDPVLEQNPFDVFLSPESPTNQTTVIVRADDLDL
NOV50i VARDCSIQGSRSTTVIIKVYVTDVNDNDPVLEQNPFDVFLSPESPTNQTTVIVRADDLDL
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h GPNGTVDSDDSPLLDDFHVHPDTGIIRTARRLDRERRDHYSFVAATLLGAVVQVEIRVND
NOV50i GPNGTVDSDDSPLLDDFNVHPDTGIIRTARRLDRERRDHYSFVAATLLGAVVQVEIRVND
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
N0V50h VNDHSPRFPLDSLQLDVSELSPPGTAFRLPVAHDPDAGLFSTQGYTLVQPSDLPKDPAGP
NOV50i VNDHSPRFPLDSLQLDVSELSPPGTAFRLPVAHDPDAGLFSTQGYTLVQPSDLPKDPAGP
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h FFQLRYRTPGPLPSPLLPGSSSPLEPLDLVLLRRLDREEAAAHRLQIEAWDGGRPRRTGL
NOV50i FFQLRYRTPGPLPSPLLPGSSSPLEPLDLVLLRRLDREEAAAHRLQIEAWDGGRPRRTGL
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h LSVELRVLDENDNPPVFEQDESRAAVREHAQPGAEVCRVRATARDVGPNGFVRYSVRARQ
NOV50i LSVELRVLDENDNPPVFEQDESRAAVREHAQPGAEVCRVRATARDVGPNGFVRYSVRARQ
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h VPGAGSGGGALGDAAYFALVVEARDGGAEHEVAAVRVSIAVLDVNDNRPAIHVLFLTEGG
NOV50i VPGAGSGGGALGDAAYFALVVEARDGGAEHEVAAVRVSIAVLDVNDNRPAIHVLFLTEGG
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h VARVSEGARPGDYVARVSVSDAGGDWEKEDEATGELGVTASDADSGLYGFIEYSLYDGFL
NOV50i VARVSEGARPGDYVARVSVSDAGGDWEKEDEATGELGVTASDADSGLYGFIEYSLYDGFL
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h SYEAPQAFRIDPHDGQICVSQDIDRERDPATYDLLVEAKDGFEIMPGASFELFEINSDTG
NOV50i SYEAPQAFRIDPHDGQICVSQDIDRERDPATYDLLVEAKDGFEIMPGASFELFEINSDTG
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h EVVTTTILDREIQEVFTLRVLVRDGGFPSLSSTTTILCTVEDENDHAPEFIVSSYDIEVL
NOV50i EVVTTTILDREIQEVFTLRVLVRDGGFPSLSSTTTILCTVEDENDHAPEFIVSSYDIEVL
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h ENQEPEVVYTVLASDMDAGNNRAVEYHIIDSSEPIFYRISSGDLGGKFSIHPRLGTIRTR
NOV50i ENQEPEVVYTVLASDMDAGNNRAVEYHIIDSSEPIFYRISSGDLGGKFSIHPRLGTIRTR
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h KPLDHETQPVVVLTVQAQLGSAPACSSTEVNITVMDVNDNHPAFLRTSDEIRISQTTPPG
NOV50i KPLDHETQPVVVLTVQAQLGSAPACSSTEVNITVMDVNDNHPAFLRTSDEIRISQTTPPG
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h TALYLARAEDRDSGRNGLIRYSIASPQPGVFAIDRALGVLFLNGSLGAGEQRELTLTLRA
NOV50i TALYLARAEDRDSGRNGLIRYSIASPQPGVFAIDRALGVLFLNGSLGAGEQRELTLTLRA
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h EDQGVHPQAALLVLTVVIEKREHSPSWTFEHLVYQVEVSTSIVTVKAFAPDSIQDSMKYS
NOV50i EDQGVHPQAALLVLTVVIEKREHSPSWTFEHLVYQVEVSTSIVTVKAFAPDSIQDSMKYS
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h IFSGNEDGVLSLCSKSGVVNCLASLSHTDFLSLKFESSVKGHQDRDKLQPIHLDDNNSKK
NOV50i IFSGNEDGVLSLCSKSGVVNCLASLSHTDFLSLKFESSVKGHQDRDKLQPIHLDDNNSKK
NOV50a ------------------------------------------------------------
NOV50b ------------------------------------------------------------
NOV50c ------------------------------------------------------------
NOV50d ------------------------------------------------------------
NOV50e ------------------------------------------------------------
NOV50f ------------------------------------------------------------
NOV50g ------------------------------------------------------------
NOV50h LCFTFPRATQALVFTGHCLSDTSLPGWVFATDLDSGLNGLIEYSILSGNQEEAFQIDALS
NOV50i LCFTFPRATQALVFTGHCLSDTSLPGWVFATDLDSGLNGLIEYSILSGNQEEAFQIDALS
NOV50a ------------------LIVQATDKGMPRLSNTTVIKVQVTDTNDNAPAFLPSEAVEIT
NOV50b ----------------------------------------------------------GS
NOV50c ----------------------------------------------------------GS
NOV50d ----------------------------------------------------------GS
NOV50e ------------------------------------------------------------
NOV50f ------------------------------------------------------------
NOV50g ----------------------------------------------------------GS
NOV50h GVITTKAILDYELTSSYSLIVQATDKGMPRLSNTTVIKVQVTDINDNAPAFLPSEAVEIT
NOV50i GVITTKAILDYELTSSYSLIVQATDKGMPRLSNTTVIKVQVTDINDNAPAFLPSEAVEIT
NOV50a EVMTISEDSLPGVIVTHVSVHDVDLNSAFIFSFAKESNPGTKFAIDQNTGVVVLVKTLDF
NOV50b EVMTISEDSLPGVIVTHVSVHDVDLNSAFIFSFAKESNPGTKFAIDQNTGVVVLVKTLDF
NOV50c EVMTISEDSLPGVIVTHVSVHDVDTNSAFIFSFAKESNPGTKFAIDQNTGVVVLVKTLDF
NOV50d EVMTISEDSLPGVIVTHVSVHDVDLNSAFIFSFAKESNPGTKFAIDQNTGVVVLVKTLDF
NOV50e ------------------------------------------------------------
NOV50f EVMTISEDSLPGVIVTHVSVHDVDLNSAFIFSFAKESNPGTKFAIDQNTGVVVLVKTLDF
NOV50g EVMTISEDSLPGVIVTHVSVHDVDLNSAFIFSFAKESNPGTKFAIDQNTGVVVLVKTLDF
NOV50h EVMTISEDSLPGVIVTHVSVHDVDLNSAFIFSFAKESNPGTKFAIDQNTGVVVLVKTLDF
NOV50i EVMTISEDSLPGVIVTHVSVHDVDLNSAFIFSFAKESNPGTKFAIDQNTGVVVLVKTLDF
NOV50a EEMTEYELLIQISDSVHYTEGALVVRVLDVNDNPPVFSQDFYQVTVPESIPVGYSVLTLS
NOV50b EEMTEYELLIQISDSVHYTEGALVVRVLDVNDNPPVFSQDFYQVTVPESIPVGYSVLTLS
NOV50c EEMTEYELLIQISDSVHYTEGALVVRVLDVNDNPPVFSQDFYQVTVPESIPVGYSVLTLS
NOV50d EEMTEYELLIQISDSVHYTEGALVVRVLDVNDNPPVFSQDFYQVTVPESIPVGYSVLTLS
NOV50e --MTEYELLIQISDSVHYTEGALVVRVLDVNDNPPVFSQDFYQVTVPESIPVGYSVLTLS
NOV50f EEMTEYELLIQISDSVHYTEGALVVRVLDVNDNPPVFSQDFYQVTVPESIPVGYSVLTLS
NOV50g EEMTEYELLIQISDSVHYTEGALVVRVLDVNDNPPVFSQDFYQVTVPESIPVGYSVLTLS
NOV50h EEMTEYELLIQISDSVHYTEGALVVRVLDVNDNPPVFSQDFYQVTVPESIPVGYSVLTLS
NOV50i EEMTEYELLIQISDSVHYTEGALVVRVLDVNDNPPVFSQDFYQVTVPESIPVGYSVLTLS
NOV50a ATDLESNENISYRILSSSKEFSIDPKNGTIFTISPVLLLDTISTTRFLVEASDGGNPDPR
NOV50b ATDLESNENISYRILSSSKEFSIDPKNGTIFTISPVLLLDTISTTQFLVEASDGGNPDLR
NOV50c ATDLESNENISYRILSSSKEFSIDPKNGTIFTISPVLLLDTISTTQFLVEASDGGNPDLR
NOV50d ATDLESNENISYRILSSSKEFSIDPKNGTIFTISPVLLLDTISTTQFLVEASDGGNPDLR
NOV50e ATDLESNENISYRILSSSKEFSIDPKNGTIFTISPVLLLDTISTTQFLVEASDGGNPDLR
NOV50f ATDLESNENISYRILSSSKEFSIDPKNGTIFTISPVLLLDTISTTQFLVEASDGGNPDLR
NOV50g ATDLESNENISYRILSSSKEFSIDPKNGTIFTISPVLLLDTISTTQFLVEASDGGNPDLR
NOV50h ATDLESNENISYRILSSSKEFSIDPKNGTIFTISPVLLLDTISTTRFLVEASDGGNPDPR
NOV50i ATDLESNENISYRILSSSKEFSIDPKNGTIFTISPVLLLDTISTTRFLVEASDGGNPDPR
NOV50a ALTLVEIGIEDMNNYAPEFTVKSYNLSLSEDALVGSTLVTFSNIDHDWTRENTYVEYSII
NOV50b ALTLVEIGIEDMNNYAPEFTVKSYNLSLSEDALVGSTLVTFSNIDHDWTRENTYVEYSII
NOV50c ALTLVEIGIEDMNNYAPEFTVKSYNLSLSEDALVGSTLVTFSNIDHDWTRENTYVEYSII
NOV50d ALTLVEIGIEDMNNYAPEFTVKSYNLSLSEDALVGSTLVTFSNIDHDWTRENTYVEYSII
NOV50e ALTLVEIGIEDMNNYAPEFTVKSYNLSLSEDALVGSTLVTFSNIDHDWTRENTYVEYSII
NOV50f ALTLVEIGIEDMNNYAPEFTVKSYNLSLSEDALVGSTLVTFSNIDHDWTRENTYVEYSII
NOV50g ALTLVEIGIEDMNNYAPEFTVKSYNLSLSEDALVGSTLVTFSNIDHDWTRENTYVEYSII
NOV50h ALTLVEIGIEDMNNYAPEFTVKSYNLSLSEDALVGSTLVTFSNIDHDWTRENTYVEYSII
NOV50i ALTLVEIGIEDMNNYAPEFTVKSYNLSLSEDALVGSTLVTFSNIDHDWTRENTYVEYSII
NOV50a SGNSQNNFHVETKFFHSEYPYKQVGYLVLLHSLDREASASHELVILASDSGCPPLSSTAV
NOV50b SGNSQNNFHVETKFFHSEYPYKQVGYLVLLHSLDREASASHELVILASDSGCPPLSSTAV
NOV50c SGNSQNNFHVETKFFHSEYPYKQVGYLVLLHSLDREASASHELVILASDSGCPPLSSTAV
NOV50d SGNSQNNFHVETKFFHSEYPYKQVGYLVLLHSLDREASASHELVILASDSGCPPLSSTAV
NOV50e SGNSQNNFHVETKFFHSEYPYKQVGYLVLLHSLDREASASHELVILASDSGCPPLSSTAV
NOV50f SGNSQNNFHVETKFFHSEYPYKQVGYLVLLHSLDREASASHELVILASDSGCPPLSSTAV
NOV50g SGNSQNNFHVETKFFHSEYPYKQVGYLVLLHSLDREASASHELVILASDSGCPPLSSTAV
NOV50h SGNSQNNFHVETKFFHSEYPYKQVGYLVLLHSLDREASASHELVILASDSGCPPLSSTAV
NOV50i SGNSQNNFHVETKFFHSEYPYKQVGYLVLLHSLDREASASHELVILASDSGCPPLSSTAV
NOV50a ISIQVLDVNDNPPNFSSLSYHTHVKESTPLGSHITVVSANDRDTGSHAEIIYNIISGNEK
NOV50b ISIQVLDVNDNPPNFSSLSYHTHVKESTPLGSHITVVSANDRDTGSHAEIIYNIISGNEK
NOV50c ISIQVLDVNDNPPNFSSLSYHTHVKESTPLGSHITVVSANDRDTGSHAEITYNIISGNEK
NOV50d ISIQVLDVNDNPPNFSSLSYHTHVKESTPLGSHITVVSANDRDTGSHAEIIYNIISGNEK
NOV50e ISIQVLDVNDNPPNFSSLSYHTHVKESTPLGSHITVVSANDRDTGSHAEIIYNIISGNEK
NOV50f ISIQVLDVNDNPPNFSSLSYHTHVKESTPLGSHITVVSANDRDTGSHAEIIYNIISGNEK
NOV50g ISIQVLDVNDNPPNFSSLSYHTHVKESTPLGSHITVVSANDRDTGSHAEIIYNIISGNEK
NOV50h ISIQVLDVNDNPPNFSSLSYHTHVKESTPLGSHITVVSANDRDTGSHAEIIYNIISGNEK
NOV50i ISIQVLDVNDNPPNFSSLSYHTHVKESTPLGSHITVVSANDRDTGSHAETIYNIISGNEK
NOV50a GHFYLEENTGVLYLIKPLDYEKMTKFTLTVQASDAEKKHFSFAVVFVSVLDDNDHAPQFM
NOV50b GHFYLEENTGVLYLIKPLDYEKMTKFTLTVQASDAEKKHFSFAVVFVSVLDDNDHAPQFM
NOV50c GHFYLEENTGVLYLIKPLDYEKMTKFTLTVQASDAEKKHFSFAVVFVSVLDDNDHAPQFM
NOV50d GHFYLEENTGVLYLIKPLDYEKMTKFTLTVQASDAEKKHFSFAVVFVSVLDDNDHAPQFM
NOV50e GHFYLEENTGVLYLIKPLDYEKMTKFTLTVQASDAEKKHFSFAVVFVSVLDDNDHAPQFM
NOV50f GHFYLEENTGVLYLIKPLDYEKMTKFTLTVQASDAEKKHFSFAVVFVSVLDDNDHAPQFM
NOV50g GHFYLEENTGVLYLIKPLDYEKMTKFTLTVQASDAEKKHFSFAVVFVSVLDDNDHAPQFM
NOV50h GHFYLEENTGVLYLIKPLDYEKMTKFTLTVQASDAEKKHFSFAVVFVSVLDDNDHAPQFM
NOV50i GHFYLEENTGVLYLIKPLDYEKMTKFTLTVQASDAEKKHFSFAVVFVSVLDDNDHAPQFM
NOV50a FSSFSCIVPENLPISSTICSINALDFDAGPYGELTYSIVSPCFLTHGMSYDHDLFLIDPL
NOV50b FSSFSCIVPENLPISSTIRSINALDFDAGPYGELTYSIVSPCFLTHGMSYDHDLFLIDPL
NOV50c FSSFSCIVPENLPISSTICSINALDFDAGPYGELTYSIVSPCFLTHGMSYDHDLFLIDPL
NOV50d FSSFSCIVPENLPISSTICSINALDFDAGPYGELTYSIVSPCFLTHGMSYDHDLFLIDPL
NOV50e FSSFSCIVPENLPISSTICSTNALDFDAGPYGELTYSIVSPCFLTHGMSYDHDLFLIDPL
NOV50f FSSFSCIVPENLPISSTICSINALDFDAGPYGELTYSIVSPCFLTHGMSYDHDLFLIDPL
NOV50g FSSFSCIVPENLPISSTICSINALDFDAGPYGELTYSIVSPCFLTHGMSYDHDLFLIDPL
NOV50h FSSFSCIVPENLPISSTICSINALDFDAGPYGELTYSIVSPCFLTHGMSYDHDLFLIDPL
NOV50i FSSFSCIVPENLPISSTICSINALDFDAGPYGELTYSIVSPCFLTHGMSYDHDLFLIDPL
NOV50a TGDIHAKQILDYENGNKYCLTVQAKDKGDATASLVVWVDIEGIDEFEPIFTQDQYFFTLP
NOV50b TGDIHAKQILDYENGNKYCLTVQAKDKGDATASLVVWVDIEGIDEFEPIFTQDQYFFTLP
NOV50c TGDIHAKQILDYENGNKYCLTVQAKDKGDATASLVVWVDIEGIDEFEPIFTQDQYFFTLP
NOV50d TGDIHAKQILDYENGNKYCLTVQAKDKGDATASLVVWVDIEGIDEFEPIFTQDQYFFTLP
NOV50e TGDIHAKQILDYENGNKYCLTVQAKDKGDATASLVVWVDIEGIDEFEPIFTQDQYFFTLP
NOV50f TGDIHAKQILDYENGNKYCLTVQAKDKGDATASLVVWVDIEGIDEFEPIFTQDQYFFTLP
NOV50g TGDIHAKQILDYENGNKYCLTVQAKDKGDATASLVVWVDIEGIDEFEPIFTQDQYFFTLP
NOV50h TGDIHAKQILDYENGNKYCLTVQAKDKGDATASLVVWVDIEGIDEFEPIFTQDQYFFTLP
NOV50i TGDIHAKQILDYENGNKYCLTVQAKDKGDATASLVVWVDIEGIDEFEPIFTQDQYFFTLP
NOV50a EKNKDRQLIGRVEASDADAGIDGVILYSLGTSSPFFSVNRTNGNIYLIRALPLIKSQLNK
NOV50b EKNKDRQLIGRVEASDADAGIDGVILYSLGTSSPFFSVNKTNGNIYLIRALPLIKSQLNK
NOV50c EKNKDRQLIGRVEASDADAGIDGVILYSLGTSSPFFSVNKTNGNIYLIRALPLIKSQLNK
NOV50d EKNKDRQLIGRVEASDADAGIDGVILYSLGTSSPFFSVNKTNGNIYLIRALPLIKSQLNK
NOV50e EKNKDRQLIGRVEASDADAGIDGVILYSLGTSSPSLSVNKTNGNIYLIPALPLIKSQLNK
NOV50f EKNKDRQLIGRVEASDADAGIDGVILYSLGTSSPFFSVNKTNGNTYLIRALPLIKSQLNK
NOV50g EKNKDRQLIGRVEASDADAGIDGVILYSLGTSSPFFSVNKTNGNIYLIRALPLIKSQLNK
NOV50h EKNKDRQLIGRVEASDADAGIDGVILYSLGTSSPFFSVNRTNGNIYLIRALPLIKSQLNK
NOV50i EKNKDRQLIGRVEASDADAGIDGVILYSLGTSSPFFSVNRTNGNIYLIRALPLIKSQLNK
NOV50a EDTLEMKIIAHSPKSDSKFASCTVFVNVSFSSEGTPLAVFASSFSISLVVSFLVFLILIC
NOV50b EDTLEMKIIAHSPKSDSKFASCTVFVNVSFSSEGTPLAVFASSFSISLVVSFLVFLILIC
NOV50c EDTLEMKIIAHSPKSDSKFASCTVFVNVSFSSEGTPLAVFASSFSISLVVSFLVFLTLIC
NOV50d EDTLEMKIIAHSPKSDSKFASCTVFVNVSVD-----------------------------
NOV50e EDTLEMKIIAHSPKSDSKFASCTVFVNVSFSSEGTPLAVFASSFSISLVVSFLVFLTLIC
NOV50f EDTLEMKIIAHSPKSDSKFASCTVFVNVSFSSEGTPLAVFASSFSISLVVSFLVFLILIC
NOV50g EDTLEMKIIAHSPKSDSKFASCTVFVNVSVD-----------------------------
NOV50h EDTLEMKTTAHSPKSDSKFASCTVFVNVSFSSEGTPLAVFASSFSISLVVSFLVFLILIC
NOV50i EDTLEMKITAHSPKSDSKFASCTVFVNVSFSSEGTPLAVFASSFSISLVVSFLVFLILIC
N0V50a ILIVMILRHKQKDTINNYEEKKTSSLDADLRVTRDASVLKAFQKTDDCSNEVVPVDATPE
NOV50b ILIVNILRHKQKDTINNYEEKKTSSLDADLRVTRDASVLKAFQKTDDCSNEVVPVDATPE
NOV50c ILIVMILRHKQKDTINNYEEKKTSSLDADLRVTRDASVLKAFQKTDDCSNEVVPVDATPE
NOV50d ------------------------------------------------------------
NOV50e ILIVMILRHKQKDTINNYEEKKTSSLDADLRVTRDASVLKAFQKTDDCSNEVVPVDATPE
NOV50f ILIVMILRHKQKDTINNYEEKKTSSLDADLRVTRDASVLKAFQKTDDCSNEVVPVDATPE
NOV50g ------------------------------------------------------------
NOV50h ILIVMILRHKQKDTINNYEEKKTSSLDADLRVTRDASVLKAFQKTDDCSNEVVPVDATPE
NOV50i ILIVMILRHKQKDTINNYEEKKTSSLDADLRVTRDASVLKAFQKTDDCSNEVVPVDATPE
NOV50a WLSLISIMEKDIVNLYRYSNSSGHCSVEGETAEDKEIQRINEHPYRKCSDSALSDHESRV
NOV50b WLSLISIMEKDIVNLYRHSNSSGHCSVEGETAEDKEIQRINEHPYRKCSDSALSDHESRV
NOV50c WLSLISIMEKDIVNLYRHSNSSGHCSVEGETAEDKEIQRINEHPYRKCSDSALSDHESRV
NOV50d ------------------------------------------------------------
NOV50e WLSLISIMEKDTVNLYRHSNSSGHCSVEGETAEDKEIQRINEHPYRKCSDSALSDHESRV
NOV50f WLSLISIMEKDIVNLYRHSNSSGHCSVEGETAEDKEIQRINEHPYRKCSDSALSDHESRV
NOV50g ------------------------------------------------------------
NOV50h WLSLISIMEKDIVNLYRYSNSSGHCSVEGETAEDKEIQRINEHPYRKCSDSALSDHESRV
NOV50i WLSLISIMEKDIVNLYRYSNSSGHCSVEGETAEDKEIQRINEHPYRKCSDSALSDHESRV
NOV50a PDSGIPPDSDQLSCLSGETDVMVTAETAEASQTFGEGDQGEGCSTTCAQNNVLPQTVQKR
NOV50b PDSGIPRDSDQLSCLSGETDVMVTAETAEASQTFGEGDQGEGCSTTCAQNNVLPQTVQKR
NOV50c PDSGIPRDSDQLSCLSGETDVMVTAETAEASQTFGEGDQGEGCSTTCAQNNVLPQTVQKR
NOV50d ------------------------------------------------------------
NOV50e PDSGIPRDSDQLSCLSGETDVMVTAETAEASQTFGEGDQGEGCSTTCAQNNVLPQTVQKR
NOV50f PDSGIPRDSDQLSCLSGETDVMVTAETAEASQTFGEGDQGEGCSTTCAQNNVLPQTVQKR
NOV50g ------------------------------------------------------------
NOV50h PDSGIPRDSDQLSCLSGETDVMVTAETAEASQTFGEGDQGEGCSTTCAQNNVLPQTVQKR
NOV50i PDSGIPRDSDQLSCLSGETDVMVTAETAEASQTFGEGDQGEGCSTTCAQNNVLPQTVQKR
NOV50a EAKESILADVRKESVFISGDQEVRCAALSTQTTSDHDGKDNYHWNYLLSWEPKFQPLASV
NOV50b EAKESILADVRKESVFISGDQEVRCAALSTQTTSDHDGKDNYHWNYLLSWEPKFQPLASV
NOV50c EAKESILADVRKESVFISGDQEVRCAALSTQTTSDHDGKDNYHWNYLLSWEPKFQPLASV
NOV50d ------------------------------------------------------------
NOV50e EAKESILADVRKESVFISGDQEVRCAALSTQTTSDHDGKDNYHWNYLLSWEPKFQPLASV
NOV50f EAKESTLADVRKESVFISGDQEVRCAALSTQTTSDHDGKDNYHWNYLLSWEPKFQPLASV
NOV50g ------------------------------------------------------------
NOV50h EAKESILADVRKESVFISGDQEVRCAALSTQTTSDHDGKDNYHWNYLLSWEPKFQPLASV
NOV50i EAKESILADVRKESVFISGDQEVRCAALSTQTTSDHDGKDNYHWNYLLSWEPKFQPLASV
NOV50a FNDIAKLKDEHLHMPGIPKEKKSFVFPPPLITAVAQPGIKAVPPRMPAVNLGQVPPKHPR
NOV50b FNDIAKLKDEHLHMPGIPKEKKSFVFPPPLITAVAQPGIKAVPPRMPAVNLGQVPPKHPR
NOV50c FNDIAKLKDEHLHMPGIPKEKKSFVFPPPLITAVAQPGIKAVPPRMPAVNLGQVPPKHPR
NOV50d ------------------------------------------------------------
NOV50e FNDIAKLKDEHLHNPGIPKEKKSFVFPPPLITAVAQPGIKAVPPRMPAVNLGQVPPKHPR
NOV50f FNDIAKLKDEHLHMPGIPKEKKSFVFPPPLITAVAQPGIKAVPPRMPAVNLGQVPPKHPR
NOV50g ------------------------------------------------------------
NOV50h FNDIAKLKDEHLHMPGIPKEKKSFVFPPPLITAVAQPGIKAVPPRMPAVNLGQVPPKHPR
NOV50i FNDIAKLKDEHLHMPGIPKEKKSFVFPPPLITAVAQPGIKAVPPRNPAVNLGQVPPKHPR
NOV50a SPIPYHLGSLPEGMTPNFSPSLSLLTMQPPALSPLLREGELLGTHISGTCHELKAEDEVQ
NOV50b SPIPYHLGSLPEGMTPNFSPSLSLLTMQPPALSPLLREGELLGTHISGTCHELKAEDEVQ
NOV50c SPIPYHLGSLPEGMTPNFSPSLSLLTMQPPALSPLLREGELLGTHISGTCHELKAEDEVQ
NOV50d ------------------------------------------------------------
NOV50e SPIPYHLGSLPEGMTPNFSPSLSLLTMQPPALSPLLREGELLGTHISGTCHELKAEDEVQ
NOV50f SPIPYHLGSLPEGMTPNFSPSLSLLTMQPPALSPLLREGELLGTHISGTCHELKAEDEVQ
NOV50g ------------------------------------------------------------
NOV50h SPIPYHLGSLPEGMTPNFSPSLSLLTMQPPALSPLLREGELLGTHISGTCHELKAEDEVQ
NOV50i SPIPYHLGSLPEGMTPNFSPSLSLLTMQPPALSPLLREGELLGTHISGTCHELKAEDEVQ
NOV50a I--
NOV50b IVD
NOV50c IVD
NOV50d ---
NOV50e I--
NOV50f I--
NOV50g ---
NOV50h I--
NOV50i I--
NOV50a (SEQ ID NO: 1066)
NOV50b (SEQ ID NO: 1068)
NOV50c (SEQ ID NO: 1070)
NOV50d (SEQ ID NO: 1072)
NOV50e (SEQ ID NO: 1074)
NOV50f (SEQ ID NO: 1076)
NOV50g (SEQ ID NO: 1078)
NOV50h (SEQ ID NO: 1080)
NOV50i (SEQ ID NO: 1082)

Further analysis of the NOV50a protein yielded the following properties shown in Table 50C.

TABLE 50C
Protein Sequence Properties NOV50a

| | |
|---|---|
| SignalP analysis: | No Known Signal Sequence Predicted |
| PSORT II analysis: | |

| PSG: | a new signal peptide prediction method | |
|---|---|---|
| | N-region: | length 8; pos. chg 1; neg. chg 1 |
| | H-region: | length 3; peak value −4.35 |
| | PSG score: | −8.75 |

| | |
|---|---|
| GvH: | von Heijne's method for signal seq. recognition |
| | GvH score (threshold: −2.1): −9.63 |
| | possible cleavage site: between 38 and 39 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 1 | | |
| | Number of TMS(s) for threshold 0.5: 1 | | |
| | INTEGRAL | Likelihood = | Transmembrane 690-706 |
| | | −18.15 | |

| | |
|---|---|
| PERIPHERAL | Likelihood = 2.54 (at 195) |

| | |
|---|---|
| | ALOM score: −18.15 (number of TMSs: 1) |
| MTOP: | Prediction of membrane topology (Hartmann et al.) |
| | Center position for calculation: 697 |
| | Charge difference: 3.5 C(2.5)-N(−1.0) |
| | C > N: C-terminal side will be inside |

>>> membrane topology: type 1b (cytoplasmic tail 690 to 1063)

| MITDISC: | discrimination of mitochondrial targeting seq | | | |
|---|---|---|---|---|
| | R content: | 1 | Hyd Moment(75): | 8.18 |
| | Hyd Moment(95): | 10.59 | G content: | 1 |
| | D/E content: | 2 | S/T content: | 5 |

| | |
|---|---|
| | Score: −4.29 |
| Gavel: | prediction of cleavage sites for mitochondrial preseq |
| | R-2 motif at 22 PRL\|SN |

NUCDISC: discrimination of nuclear localization signals

| | |
|---|---|
| | pat4: none |
| | pat7: PKEKKSF (5) at 960 |
| | bipartite: none |
| | content of basic residues: 7.1% |
| | NLS Score: −0.04 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: found
LL at 929
LL at 1026
LL at 1037
LL at 1043
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 70.6 |
| COIL: | Lupas's algorithm to detect coiled-coil regions |
| | total: 0 residues |

Final Results (k = 9/23):

```
34.8%: nuclear
26.1%: cytoplasmic
17.4%: mitochondrial
  8.7%: vesicles of secretory system
  4.3%: vacuolar
  4.3%: peroxisomal
  4.3%: endoplasmic reticulum
```

>> prediction for CG58567-01 is nuc (k = 23)

A search of the NOV50a protein against the Geneseq database, a proprietary database that contains sequences published in patents and patent publication, yielded several homologous proteins shown in Table 50D.

TABLE 50D
Geneseq Results for NOV50a

| | | | Identities/ | | |
|---|---|---|---|---|---|
| | | NOV50a | Similarities | | |
| Geneseq | Protein/Organism/Length | Residues/ | for the | | Expect |
| Identifier | [Patent #, Date] | Match Residues | Matched Region | | Value |
| ABP70112 | Human NOV28c - Homo sapiens, | 1 . . . 1063 | 1063/1063 | (100%) | 0.0 |
| | 3361 aa. [WO200272771-A2, | 2299 . . . 3361 | 1063/1063 | (100%) | |
| | 19-SEP-2002] | | | | |
| ABP70111 | Human NOV28b - Homo sapiens, | 1 . . . 1063 | 1063/1063 | (100%) | 0.0 |
| | 3003 aa. [WO200272771-A2, | 1941 . . . 3003 | 1063/1063 | (100%) | |
| | 19-SEP-2002] | | | | |
| ABP70110 | Human NOV28a - Homo sapiens, | 1 . . . 1063 | 1063/1063 | (100%) | 0.0 |
| | 1063 aa. [WO200272771-A2, | 1 . . . 1063 | 1063/1063 | (100%) | |
| | 19-SEP-2002] | | | | |
| ABP43938 | FLJ20047 fis clone COL00577 - | 105 . . . 1063 | 959/959 | (100%) | 0.0 |
| | Homo sapiens, 959 aa. | 1 . . . 959 | 959/959 | (100%) | |
| | [WO200231111-A2, 18-APR-2002] | | | | |
| AAO26793 | Human cadherin (CAD) protein, SEQ | 1 . . . 1036 | 366/1073 | (34%) | e−148 |
| | ID No 16 - Homo sapiens, 3298 aa. | 2245 . . . 3270 | 543/1073 | (50%) | |
| | [WO200299042-A2, 12-DEC-2002] | | | | |

In a BLAST search of public sequence databases, the NOV50a protein was found to have homology to the proteins shown in the BLASTP data in Table 50E.

TABLE 50E
Public BLASTP Results for NOV50a

| | | NOV50a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| Q9NXU8 | Hypothetical protein FLJ20047 - | 105 . . . 1063 | 959/959 | (100%) | 0.0 |
| | Homo sapiens (Human), 959 aa. | 1 . . . 959 | 959/959 | (100%) | |
| Q9NRU0 | Cadherin-like protein VR8 - Homo | 189 . . . 582 | 391/394 | (99%) | 0.0 |
| | sapiens (Human), 398 aa (fragment). | 5 . . . 398 | 393/394 | (99%) | |
| Q96JQ0 | Protocadherin 16 precursor | 1 . . . 1036 | 366/1073 | (34%) | e−148 |
| | (Cadherin 19) (Cadherin fibroblast | 2245 . . . 3270 | 543/1073 | (50%) | |
| | 1) - Homo sapiens (Human), 3298 aa. | | | | |
| Q24292 | Dachsous protein precursor | 1 . . . 1037 | 328/1165 | (28%) | e−100 |
| | (Adherin) - Drosophila melanogaster | 2343 . . . 3471 | 528/1165 | (45%) | |
| | (Fruit fly), 3503 aa. | | | | |
| Q8AXB7 | Protocadherin - Brachydanio rerio | 3 . . . 605 | 206/615 | (33%) | 3e−77 |
| | (Zebrafish) (Danio rerio), 4610 aa. | 2885 . . . 3471 | 317/615 | (51%) | |

PFam analysis predicts that the NOV50a protein contains the domains shown in the Table 50F.

TABLE 50F
Domain Analysis of NOV50a

| | | Identities/ | |
|---|---|---|---|
| Pfam | NOV50a | Similarities for | Expect |
| Domain | Match Region | the Matched Region | Value |
| cadherin | 43 . . . 130 | 26/108 (24%) | 1.6e−14 |
| | | 64/108 (59%) | |
| cadherin | 144 . . . 232 | 36/108 (33%) | 2.5e−12 |
| | | 66/108 (61%) | |
| cadherin | 246 . . . 348 | 36/113 (32%) | 2e−15 |
| | | 79/113 (70%) | |
| cadherin | 362 . . . 452 | 35/107 (33%) | 8.4e−24 |
| | | 75/107 (70%) | |
| cadherin | 466 . . . 563 | 33/115 (29%) | 1.1e−11 |
| | | 67/115 (58%) | |
| cadherin | 577 . . . 671 | 26/110 (24%) | 0.17 |
| | | 60/110 (55%) | |

### Example 51

The NOV51 clone was analyzed, and the nucleotide and encoded polypeptide sequences are shown in Table 51A.

TABLE 51 A
NOV51 Sequence Analysis

| | | |
|---|---|---|
| NOV51 a, CG59534-03 | SEQ ID NO:1083 | 3333 bp |
| DNA Sequence | ORF Start: ATG at 15 | ORF Stop: TAG at 3219 |

GCGCTCCAGACAAGATGGCGCGGCCGGTCCGGGGAGGGCTCGGGGCCCCGCGCCGCTCGCCTTGCCTT
CTCCTTCTCTGGCTGGTTTTGGTTCGGCTGGAGCCGGTGACCGCCGCGGCCGGCCCGCGGGCGCCCTG
CGCGGCCGCCTGCACTTGCGCTGGGGACTCGCTGGACTGCGGTGGGCGCGGGCTGGCTGCGTTGCCCG
GGGACCTGCCCTCCTGGACGCGGAGCCTAAACCTGAGTTACAACAAACTCTCTGAGATTGACCCTGCT
GGTTTTGAGGACTTGCCGAACCTACAGGAAGTGCAGCACAACAAGATTCGCAGCGTGGAGGGGAGCCA
GCTGAAGGCCTACCTTTCCTTAGAAGTGTTAGATCTGAGTTTGAACAACATCACGGAAGTGCGGAACA
CCTGCTTTCCACACGGACCGCCTATAAAGGAGCTCAACCTGGCAGGCAATCGGATTGGCACCCTGGAG
TTGGGAGCATTTGATGGTCTGTCACGGTCGCTGCTAACTCTTCGCCTGAGCAAAAACAGGATCACCCA
GCTTCCTGTAAGAGCATTCAAGCTACCCAGGCTGACACAACTGGACCTCAATCGGAACAGGATTCGGC
TGATAGAGGGCCTCACCTTCCAGGGGCTCAACAGCTTGGAGGTGCTGAAGCTTCAGCGAAACAACATC
AGCAAACTGACAGATGGGGCCTTCTGGGGACTGTCCAAGATGCATGTGCTGCACCTGGAGTACAACAG
ATTCCATCGCTCGCATTCACCGCAAGGGCTGGAGCTTCTGCCAGAAGCTGCATGAGTTGGTCCTGTCC
TTCAACAACCTGACACGGCTGGACGAGGAGAGCCTGGCCGAGCTGAGCAGCCTGAGTGTCCTGCGTCT
CAGCCACAATTCCATCAGCCACATTGCGGAGGGTGCCTTCAAGGGACTCAGGAGCCTGCGAGTCTTGG
ATCTGGACCATAACGAGATTTCGGGCACAATAGAGGACACGAGCGGCGCCTTCTCAGGGCTCGACAGC
CTCAGCAAGCTGACTCTGTTTGGAAACAAGATCAAGTCTGTGGCTAAGAGAGCATTCTCGGGGCTGGA
AGGCCTGGAGCACCTGAACCTTGGAGGGAATGCGATCAGATCTGTCCAGTTTGATGCCTTTGTGAAGA
TGAAGAATCTTAAAGAGCTCCATATCAGCAGCGACAGCTTCCTGTGTGACTGCCAGCTGAAGTGGCTG
CCCCCGTGGCTAATTGGCAGGATGCTGCAGGCCTTTGTGACAGCCACCTGTGCCCACCCAGAATCACT
GAAGGGTCAGAGCATTTTCTCTGTGCCACCAGAGAGTTTCGTGTGCGATGACTTCCTGAAGCCACAGA
TCATCACCCAGCCAGAAACCACCATGGCTATGGTGGGCAAGGACATCCGGTTTACATGCTCAGCAGCC
AGCAGCAGCAGCTCCCCCATGACCTTTGCCTGGAAGAAAGACAATGAAGTCCTGACCAATGCAGACAT
GGAGAACTTTGTCCACGTCCACGCGCAGGACGGGGAAGTGATGGAGTACACCACCATCCTGCACCTCC
GTCAGGTCACTTTCGGGCACGAGGGCCGCTACCAATGTGTCATCACCAACCACTTTGGCTCCACCTAT
TCACATAAGGCCAGGCTCACCGTGAATGTGTTGCCATCATTCACCAAAACGCCCCACGACATAACCAT
CCGGACCACCACCGTGGCCCGCCTCGAATGTGCTGCCACAGGTCACCCAAACCCTCAGATTGCCTGGC
AGAAGGATGGAGGCACGGATTTCCCCGCTGCCCGTGAGCGACGCATGCATGTCATGCCGGATGACGAC
GTGTTTTTCATCACTGATGTGAAAATAGATGACGCAGGGGTTTACAGCTGTACTGCTCAGAACTCAGC
CGGTTCTATTTCAGCTAATGCCACCCTGACTGTCCTAGAGACCCCATCCTTGGTGGTCCCCTTGGAAG
ACCGTGTGGTATCTGTGGGAGAAACAGTGGCCCTCCAATGCAAAGCCACGGGGAACCCTCCGCCCCGC
ATCACCTGGTTCAAGGGGGACCGCCCGCTGAGCCTCACTGAGCGGCACCACCTGACCCCTGACAACCA
GCTCCTGGTGGTTCAGAACGTGGTGGCAGAGGATGCGGGCCGATATACCTGTGAGATGTCCAACACCC
TGGGCACGGAGCGAGCTCACAGCCAGCTGAGCGTCCTGCCCGCAGCAGGCTGCAGGAAGGATGGGACC
ACGGTAGGCATCTTCACCATTGCTGTCGTGAGCAGCATCGTCCTGACGTCACTGGTCTGGGTGTGCAT
CATCTACCAGACCAGGAAGAAGAGTGAAGAGTACAGTGTCACCAACACAGATGAAACCGTCGTGCCAC
CAGATGTTCCAAGCTACCTCTCTTCTCAGGGGACCCTTTCTGACCGACAAGAAACCGTGGTCAGGACC
GAGGGTGGCCCTCAGGCCAATGGGCACATTGAGAGCAATGGTGTGTGTCCAAGAGATGCAAGCCACTT
TCCAGAGCCCGACACTCACAGCGTTGCCTGCAGGCAGCCAAAGCTCTGTGCTGGGTCTGCGTATCACA
AAGAGCCGTGGAAAGCGATGGAGAAAGCTGAAGGGACACCTGGGCCACATAAGATGGAACACGGTGGC
CGGGTCGTATGCAGTGACTGCAACACCGAAGTGGACTGTTACTCCAGGGGACAAGCCTTCCACCCCCA
GCCTGTGTCCAGAGACAGCGCACAGCCAAGTGCGCCAAATGGCCCGGAGCCGGGTGGGAGTGACCAAG
AGCATTCTCCACATCACCAGTGCAGCAGGACTGCCGCTGGGTCCTGCCCCGAGTGCCAAGGGTCGCTC
TACCCCAGTAACCACGATAGAATGCTGACGGCTGTGAAGAAAAAGCCAATGGCATCTCTAGATGGGAA
AGGGGATTCTTCCTGGACTTTAGCAAGGTTGTATCACCCGGACTCCACAGAGCTACAGCCTGCATCTT
CATTAACTTCAGGCAGTCCAGAGCGCGCGGAAGCCCAGTACTTGCTTGTTTCCAATGGCCACCTCCCC
AAAGCATGTGACGCCAGTCCCGAGTCCACGCCACTGACAGGACAGCTCCCCGGGAAACAGAGGGTGCC
ACTGCTGTTGGCACCAAAAAGCTAGGTTTTGTCTACCTCAGTTCTTGTCATACCAATCTCTACGGGAA
AGAGAGGTAGGAGAGGCTGCGAGGAAGCTTGGGTTCAAGCGTCACTCATCTGTACATAGTTGT
AACTCC

| | | | |
|---|---|---|---|
| NOV51a, CG59534-03 | SEQ ID NO:1084 | 1068 aa | MW at 116452.8 kD |
| Protein Sequence | | | |

MARPVRGGLGAPRRSPCLLLLWLVLVRLEPVTAAAGPRAPCAAACTCAGDSLDCGGRGLAALPGDLPS
WTRSLNLSYNKLSEIDPAGFEDLPNLQEVQHNKIRSVEGSQLKAYLSLEVLDLSLNNITEVRNTCFPH
GPPIKELNLAGNRIGTLELGAFDGLSRSLLTLRLSKNRITQLPVRAFKLPRLTQLDLNRNRIRLIEGL
TFQGLNSLEVLKLQRNNISKLTDGAFWGLSKMHVLHLEYNSLVEVNSGSLYGLTALHQLHLSNNSIAR
IHRKGWSFCQKLHELVLSFNNLTRLDEESLAELSSLSVLRLSHNSISHIAEGAFKGLRSLRVLDLDHN
EISGTIEDTSGAFSGLDSLSKLTLFGNKIKSVAKRAFSGLEGLEHLNLGGNAIRSVQFDAFVKMKNLK
ELHISSDSFLCDCQLKWLPPWLIGRMLQAFVTATCANPESLKGQSIFSVPPESFVCDDFLKPQIITQP
ETTMAMVGKDIRFTCSAASSSSSPMTFAWKKDNEVLTNADMENFVHVHAQDGEVMEYTTILHLRQVTF
GHEGRYQCVITNHFGSTYSHKARLTVNVLPSFTKTPHDITIRTTTVARLECAATGHPNPQIAWQKDGG
TDFPAARERRMHVMPDDDVFFITDVKIDDAGVYSCTAQNSAGSISANATLTVLETPSLVVPLEDRVVS
VGETVALQCKATGNPPPRITWFKGDRPLSLTERHHLTPDNQLLVVQNVVAEDAGRYTCEMSNTLGTER
AHSQLSVLPAAGCRKDGTTVGIFTIAVVSSIVLTSLVWVCIIYQTRKKSEEYSVTNTDETVVPPDVPS
YLSSQGTLSDRQETVVRTEGGPQANGHIESNGVCPRDASHFPEPDTHSVACRQPKLCAGSAYHKEPWK
AMEKAEGTPGPHKMEHGGRVVCSDCNTEVDCYSRGQAFHPQPVSPDSAQPSAPNGPEPGGSDQEHSPH
HQCSRTAAGSCPECQGSLYPSNHDRMLTAVKKKPMASLDGKGDSSWTLARLYHPDSTELQPASSLTSG
SPERAEAQYLLVSNGHLPKACDASPESTPLTGQLPGKQRVPLLLAPKS

| | | |
|---|---|---|
| NOV51b, CG59534-01 | SEQ ID NO:1085 | 5101 bp |
| DNA Sequence | ORF Start: ATG at 77 | ORF Stop: TAG at 3308 |

ATCCTCCTACCTCAGTCTACTGAGTAGCTAGGACTACAGGCATGTGCCACCACACCTGGCTAGTTTTT
ATTTTTTGATGAGATGGGATCTTGCTGTGTTGCCCAGACTGGTCTTGATCTCCTGGGCTCAAGTGATC
CTCCTGCCTTGGCCTGCGGCCGCCTGCACTTGCGCTGGGGACTCGCTGGACTGCGGTGGGCGCGGGCT
GGCTGCGTTGCCCGGGGACCTGCCCTCCTGGACGCGGAGCCTAAACCTGAGTTACAACAAACTCTCTG
AGATTGACCCTGCTGGTTTTGAGGACTTGCCGAACCTACAGGAAGTGTACCTCAATAATAATGAGTTG
TCGCAGCGTGGAGGGGAGCCAGCTGAAGGCCTACCTTTCCTTAGAAGTGTTAGATCTGAGTTTGAACA
ACATCACGGAAGTGCGGAACACCTGCTTTCCACACGGACCGCCTATAAAGGAGCTGAACCTGGCAGGC
AATCGGATTGGCACCCTGGAGTTGGGAGCATTTGATGGTCTGTCACGGTCGCTGCTAACTCTTCGCCT
GAGCAAAAACAGGATCACCCAGCTTCCTGTAAGAGCATTCAAGCTACCCAGGCTGACACAACTGGACC
TCAATCGGAACAGGATTCGGCTGATAGAGGGCCTCACCTTCCAGGGGCTCAACAGCTTGGAGGTGCTG
AAGCTTCAGCGAAACAACATCAGCAAACTGACAGATGGGGCCTTCTGGGGACTGTCCAAGATGCATGT
GCTGCACCTGGAGTACAACAGCCTGGTAGAAGTGAACAGCGGCTCGCTCTACGGCCTCACGGCCCTGC
ATCAGCTCCACCTCAGCAACAATTCCATCGCTCGCATTCACCGCAAGGGCTGGAGCTTCTGCCAGAAG
CTGCATGAGTTGGTCCTGTCCTTCAACAACCTGACACGGCTGGACGAGGAGAGCCTGGCCGAGCTGAG
CAGCCTGAGTGTCCTGCGTCTCAGCCACAATTCCATCAGCCACATTGCGGAGGGTGCCTTCAAGGGAC
TCAGGAGCCTGCGAGTCTTGGATCTGGACCATAACGAGATTTCGGGCACAATAGAGGACACGAGCGGC
GCCTTCTCAGGGCTCGACAGCCTCAGCAAGCTGACTCTGTTTGGAAACAAGATCAAGTCTGTGGCTAA
GAGAGCATTCTCGGGGCTGGAAGGCCTGGAGCACCTGAACCTTGGAGGGAATGCGATCAGATCTGTCC
AGTTTGATGCCTTTGTGAAGATGAAGAATCTTAAAGAGCTCCATATCAGCAGCGACAGCTTCCTGTGT
GACTGCCAGCTGAAGTGGCTGCCCCCGTGGCTAATTGGCAGGATGCTGCAGGCCTTTGTGACAGCCAC
CTGTGCCCACCCAGAATCACTGAAGGGTCAGAGCATTTTCTCTGTGCCACCAGAGAGTTTCGTGTGCG
ATGACTTCCTGAAGCCACAGATCATCACCCAGCCAGAAACCACCATGGCTATGGTGGGCAAGGACATC
CGGTTTACATGCTCAGCAGCCAGCAGCAGCAGCTCCCCCATGACCTTTGCCTGGAAGAAAGACAATGA
AGTCCTGACCAATGCAGACATGGAGAACTTTGTCCACGTCCACGCGCAGGACGGGGAAGTGATGGAGT
ACACCACCATCCTGCACCTCCGTCAGGTCACTTTCGGGCACGAGGGCCGCTACCAATGTGTCATCACC
AACCACTTTGGCTCCACCTATTCACATAAGGCCAGGCTCACCGTGAATGTGTTGCCATCATTCACCAA
AACGCCCCACGACATAACCATCCGGACCACCACCATGGCCCGCCTCGAATGTGCTGCCACAGGTCACC
CAAACCCTCAGATTGCCTGGCAGAAGGATGGAGGCACGGATTTCCCCGCTGCCCGTGAGCGACGCATG
CATGTCATGCCGGATGACGACGTGTTTTTCATCACTGATGTGAAAATAGATGACGCAGGGGTTTACAG
CTGTACTGCTCAGAACTCAGCCGGTTCTATTTCAGCTAATGCCACCCTGACTGTCCTAGAGACCCCAT
CCTTGGTGGTCCCCTTGGAAGACCGTGTGGTATCTGTGGGAGAAACAGTGGCCCTCCAATGCAAAGCC
ACGGGGAACCCTCCGCCCCGCATCACCTGGTTCAAGGGGGACCGCCCGCTGAGCCTCACTGAGCGGCA
CCACCTGACCCCTGACAACCAGCTCCTGGTGGTTCAGAACGTGGTGGCAGAGGATGCGGGCCGATATA
CCTGTGAGATGTCCAACACCCTGGGCACGGAGCGAGCTCACAGCCAGCTGAGCGTCCTGCCCGCAGCA
GGCTGCAGGAAGGATGGGACCACGGTAGGCATCTTCACCATTGCTGTCGTGAGCAGCATCGTCCTGAC
GTCACTGGTCTGGGTGTGCATCATCTACCAGACCAGGAAGAAGAGTGAAGAGTACAGTGTCACCAACA
CAGATGAAACCGTCGTGCCACCAGATGTTCCAAGCTACCTCTCTTCTCAGGGGACCCTTTCTGACCGA
CAAGAAACCGTGGTCAGGACCGAGGGTGGCCCTCAGGCCAATGGGCACATTGAGAGCAATGGTGTGTG
TCCAAGAGATGCAAGCCACTTTCCAGAGCCCGACACTCACAGCGTTGCCTGCAGGCAGCCAAAGCTCT
GTGCTGGGTCTGCGTATCACAAAGAGCCGTGGAAAGCGATGGAGAAAGCTGAAGGGACACCTGGGCCA
CATAAGATGGAACACGGTGGCCGGGTCGTATGCAGTGACTGCAACACCGAAGTGGACTGTTACTCCAG
GGGACAAGCCTTCCACCCCCAGCCTGTGTCCAGAGACAGCGCACAGCCAAGTGCGCCAAATGGCCCGG
AGCCGGGTGGGAGTGACCAAGAGCATTCTCCACATCACCAGTGCAGCAGGACTGCCGCTGGGTCCTGC
CCCGAGTGCCAAGGGTCGCTCTACCCCAGTAACCACGATAGAATGCTGACGGCTGTGAAGAAAAAGCC
AATGGCATCTCTAGATGGGAAAGGGGATTCTTCCTGGACTTTAGCAAGGTTGTATCACCCGGACTCCA
CAGAGCTACAGCCTGCATCTTCATTAACTTCAGGCAGTCCAGAGCGCGCGGAAGCCCAGTACTTGCTT
GTTTCCAATGGCCACCTCCCCAAAGCATGTGACGCCAGTCCCGAGTCCACGCCACTGACAGGACAGCT
CCCCGGGAAACAGAGGGTGCCACTGCTGTTGGCACCAAAAAGCTAGGTTTTGTCTACCTCAGTTCTTG
TCATACCAATCTCTACGGGAAAGAGAGGTAGGAGAGGCTGCGAGGAAGCTTGGGTTCAAGCGTCACTC
ATCTGTACATAGTTGTAACTCCCATGTGGAGTATCAGTCGCTCACAGGACTTGGATCTGAAGCACAGT
AAACGCAAGAGGGGATTTGTGTACAAAAGGCAAAAAAAAGTATTTGATATCATTGTACATAAGAGTTT
TCAGAGATTTCATATATATCTTTTACAGAGGCTATTTTAATCTTTAGTGCATGGTTAACAGAAAAAAA
TTATACAATTTTGACAATATTATTTTTCGTATCAGGTTGCTGTTTAATTTTGGAGGGGGTGGGGAAAT
AGTTCTGGTGCCTTAACGCATGGCTGGAATTTATAGAGGCTACAACCACATTTGTTCACAGGAGTTTT
TGGTGCGGGGTGGGAAGGATGGAAGGCCTTGGATTTATATTGCACTTCATAGACCCCTAGGCTGCTGT
GCGGTGGGACTCCACATGCGCCGGAAGGAGCTTCAGGTGAGCACTGCTCATGTGTGGATGCCCCTGCA
ACAGGCTTCCCTGTCTGTAGAGCCAGGGGTGCAAGTGCCATCCACACTTGCAGTGAATGGCTTTTCCT
TTTAGGTTTAAGTCCTGTCTGTCTGTAAGGCGTAGAATCTGTCCGTCTGTAAGGCGTAGAATGAGGGT
TGTTAATCCATCACAAGCAAAAGGTCAGAACAGTTAAACACTGCCTTTCCTCCTCCTCTTATTTTATG
ATAAAAGCAAATGTGGCCTTCTCAGTATCATTCGATTGCTATTTGAGACTTTTAAATTAAGGTAAAGG
CTGCTGGTGTTGGTACCTGTGGATTTTTCTATACTGATGTTTTCGTTTTGCCAATATAATGAGTATTA
CATTGGCCTTGGGGGACAGAAAGGAGGAAGTTCTGACTTTTCAGGGCTACCTTATTTCTACTAAGGAC
CCAGAGCAGGCCTGTCCATGCCATTCCTTCGCACAGATGAAACTGAGCTGGGACTGGAAAGGACAGCC
CTTGACCTGGGTTCTGGGTATAATTTGCACTTTTGAGACTGGTAGCTAACCATCTTATGAGTGCCAAT
GTGTCATTTAGTAAAACTTAAATAGAAACAAGGTCCTTCAAATGTTCCTTTGGCCAAAAGCTGAAGGG
AGTTACTGAGAAAATAGTTAACAATTACTGTCAGGTGTCATCACTGTTCAAAAGGTAAGCACATTTAG
AATTTTGTTCTTGACAGTTAACTGACTAATCTTACTTCCACAAAATATGTGAATTTGCTGCTTCTGAG
AGGCAATGTGAAAGAGGGAGTATTACTTTTATGTACAAAGTTATTTATTTATAGAAATTTTGGTACAG
TGTACATTGAAAACCATGTAAAATATTGAAGTGTCTAACAAATGGCATTGAAGTGTCTTTAATAAAGG
TTCATTTATAAATGTCAGTATAGTTGGTGGTCCTTCTTTTACAAACGCAGTCATTCTGCCTTTAATTA
TCTTCCCCCAAAAAAGAAAAAAAAAATAGGCGAAGCAAAATCACATACTGTTTGTTTGCTCCAGGGCA
GACAACACTGCTAGATTCCTGACATTTTGTTTTGAATTTTTCTACACCTGGAGCTTGTTAGTCAAGGT
CTAAAATCCCTAAGTGTGGTGACCTTTCCATTTCATCCTGCCTTTTCAAAGCTGGCCCAGGCCCTCCT
TTCAGTCTGACATGAGAATGGCGAGAATGGCTCACCCACCGTGCCCTCCTGCACGAAGCCAG
CTGGGCC

| | | | |
|---|---|---|---|
| NOV51b, CG59534-01 | SEQ ID NO:1086 | 1077 aa | MW at 117735.2 kD |
| Protein Sequence | | | |

MRWDLAVLPRLVLISWAQVILLPWPAAACTCAGDSLDCGGRGLAALPGDLPSWTRSLNLSYNKLSEID
PAGFEDLPNLQEVYLNNNELTAVPSLGAASSHVVSLFLQHNKIRSVEGSQLKAYLSLEVLDLSLNNIT
EVRNTCFPHGPPIKELNLAGNRIGTLELGAFDGLSRSLLTLRLSKNRITQLPVRAFKLPRLTQLDLNR
NRIRLIEGLTFQGLNSLEVLKLQRNNISKLTDGAFWGLSKNHVLHLEYNSLVEVNSGSLYGLTALHQL
HLSNNSIARIHRKGWSFCQKLHELVLSFNNLTRLDEESLAELSSLSVLRLSHNSISHIAEGAFKGLRS
LRVLDLDHNEISGTIEDTSGAFSGLDSLSKLTLFGNKIKSVAKRAFSGLEGLEHLNLGGNAIRSVQFD
AFVKMKNLKELHISSDSFLCDCQLKWLPPWLIGRMLQAFVTATCAHPESLKGQSIFSVPPESFVCDDF
LKPQIITQPETTMAMVGKDIRFTCSAASSSSSPMTFAWKKDNEVLTNADMENFVHVHAQDGEVMEYTT
ILHLRQVTFGHEGRYQCVITNHFGSTYSHKARLTVNVLPSFTKTPHDITIRTTTMARLECAATGHPNP
QIAWQKDGGTDFPAARERRMHVMPDDDVFFITDVKIDDAGVYSCTAQNSAGSISANATLTVLETPSLV
VPLEDRVVSVGETVALQCKATGNPPPRITWFKGDRPLSLTERHHLTPDNQLLVVQNVVAEDAGRYTCE
MSNTLGTERAHSQLSVLPAAGCRKDGTTVGIFTIAVVSSIVLTSLVWVCIIYQTRKKSEEYSVTNTDE
TVVPPDVPSYLSSQGTLSDRQETVVRTEGGPQANGHIESNGVCPRDASHFPEPDTHSVACRQPKLCAG
SAYHKEPWKAMEKAEGTPGPHKMEHGGRVVCSDCNTEVDCYSRGQAFHPQPVSRDSAQPSAPNGPEPG
GSDQEHSPHHQCSRTAAGSCPECQGSLYPSNHDRMLTAVKKKPMASLDGKGDSSWTLARLYHPDSTEL
QPASSLTSGSPERAEAQYLLVSNGHLPKACDASPESTPLTGQLPGKQRVPLLLAPKS

| | | |
|---|---|---|
| NOV51c, CG59534-02 | SEQ ID NO:1087 | 4762 bp |
| DNA Sequence | ORF Start: ATG at 15 | ORF Stop: TAG at 3294 |

GCGCTCCAGACAAGATGGCGCGGCCGGTCCGGGGAGGGCTCGGGGCCCCGCGCCGCTCGCCTTGCCTT
CTCCTTCTCTGGCTGGTTTTGGTTCGGCTGGAGCCGGTGACCGCCGCGGCCGGCCCGCGGGCGCCCTG
CGCGGCCGCCTGCACTTGCGCTGGGGACTCGCTGGACTGCGGTGGGCGCGGGCTGGCTGCGTTGCCCG
GGGACCTGCCCTCCTGGACGCGGAGCCTAAACCTGAGTTACAACAAACTCTCTGAGATTGACCCTGCT
GGTTTTGAGGACTTGCCGAACCTACAGGAAGTGTACCTCAATAATAATGAGTTGACAGCGGTACCATC
CCTGGGCGCTGCTTCATCACATGTCGTCTCTCTCTTTCTGCAGCACAACAAGATTCGCAGCGTGGAGG
GGAGCCAGCTGAAGGCCTACCTTTCCTTAGAAGTGTTAGATCTGAGTTTGAACAACATCACGGAAGTG
CGGAACACCTGCTTTCCACACGGACCGCCTATAAAGGAGCTCAACCTGGCAGGCAATCGGATTGGCAC
CCTGGAGTTGGGAGCATTTGATGGTCTGTCACGGTCGCTGCTAACTCTTCGCCTGAGCAAAAACAGGA
TCACCCAGCTTCCTGTAAGAGCATTCAAGCTACCCAGGCTGACACAACTGGACCTCAATCGGAACAGG
ATTCGGCTGATAGAGGGCCTCACCTTCCAGGGGCTCAACAGCTTGGAGGTGCTGAAGCTTCAGCGAAA
CAACATCAGCAAACTGACAGATGGGGCCTTCTGGGGACTGTCCAAGATGCATGTGCTGCACCTGGAGT
ACAACAGCCTGGTAGAAGTGAACAGCGGCTCGCTCTACGGCCTCACGGCCCTGCATCAGCTCCACCTC
AGCAACAATTCCATCGCTCGCATTCACCGCAAGGGCTGGAGCTTCTGCCAGAAGCTGCATGAGTTGGT
CCTGTCCTTCAACAACCTGACACGGCTGGACGAGGAGAGCCTGGCCGAGCTGAGCAGCCTGAGTGTCC
TGCGTCTCAGCCACAATTCCATCAGCCACATTGCGGAGGGTGCCTTCAAGGGACTCAGGAGCCTGCGA
GTCTTGGATCTGGACCATAACGAGATTTCGGGCACAATAGAGGACACGAGCGGCGCCTTCTCAGGGCT
CGACAGCCTCAGCAAGCTGACTCTGTTTGGAAACAAGATCAAGTCTGTGGCTAAGAGAGCATTCTCGG
GGCTGGAAGGCCTGGAGCACCTGAACCTTGGAGGGAATGCGATCAGATCTGTCCAGTTTGATGCCTTT
GTGAAGATGAAGAATCTTAAAGAGCTCCATATCAGCAGCGACAGCTTCCTGTGTGACTGCCAGCTGAA
GTGGCTGCCCCCGTGGCTAATTGGCAGGATGCTGCAGGCCTTTGTGACAGCCACCTGTGCCCACCCAG
AATCACTGAAGGGTCAGAGCATTTTCTCTGTGCCACCAGAGAGTTTCGTGTGCGATGACTTCCTGAAG
CCACAGATCATCACCCAGCCAGAAACCACCATGGCTATGGTGGGCAAGGACATCCGGTTTACATGCTC
AGCAGCCAGCAGCAGCAGCTCCCCCATGACCTTTGCCTGGAAGAAAGACAATGAAGTCCTGACCAATG
CAGACATGGAGAACTTTGTCCACGTCCACGCGCAGGACGGGGAAGTGATGGAGTACACCACCATCCTG
CACCTCCGTCAGGTCACTTTCGGGCACGAGGGCCGCTACCAATGTGTCATCACCAACCACTTTGGCTC
CACCTATTCACATAAGGCCAGGCTCACCGTGAATGTGTTGCCATCATTCACCAAAACGCCCCACGACA
TAACCATCCGGACCACCACCGTGGCCCGCCTCGAATGTGCTGCCACAGGTCACCCAAACCCTCAGATT
GCCTGGCAGAAGGATGGAGGCACGGATTTCCCCGCTGCCCGTGAGCGACGCATGCATGTCATGCCGGA
TGACGACGTGTTTTTCATCACTGATGTGAAAATAGATGACGCAGGGGTTTACAGCTGTACTGCTCAGA
ACTCAGCCGGTTCTATTTCAGCTAATGCCACCCTGACTGTCCTAGAGACCCCATCCTTGGTGGTCCCC
TTGGAAGACCGTGTGGTATCTGTGGGAGAAACAGTGGCCCTCCAATGCAAAGCCACGGGGAACCCTCC
GCCCCGCATCACCTGGTTCAAGGGGGACCGCCCGCTGAGCCTCACTGAGCGGCACCACCTGACCCCTG
ACAACCAGCTCCTGGTGGTTCAGAACGTGGTGGCAGAGGATGCGGGCCGATATACCTGTGAGATGTCC
AACACCCTGGGCACGGAGCGAGCTCACAGCCAGCTGAGCGTCCTGCCCGCAGCAGGCTGCAGGAAGGA
TGGGACCACGGTAGGCATCTTCACCATTGCTGTCGTGAGCAGCATCGTCCTGACGTCACTGGTCTGGG
TGTGCATCATCTACCAGACCAGGAAGAAGAGTGAAGAGTACAGTGTCACCAACACAGATGAAACCGTC
GTGCCACCAGATGTTCCAAGCTACCTCTCTTCTCAGGGGACCCTTTCTGACCGACAAGAAACCGTGGT
CAGGACCGAGGGTGGCCCTCAGGCCAATGGGCACATTGAGAGCAATGGTGTGTGTCCAAGAGATGCAA
GCCACTTTCCAGAGCCCGACACTCACAGCGTTGCCTGCAGGCAGCCAAAGCTCTGTGCTGGGTCTGCG
TATCACAAAGAGCCGTGGAAAGCGATGGAGAAAGCTGAAGGGACACCTGGGCCACATAAGATGGAACA
CGGTGGCCGGGTCGTATGCAGTGACTGCAACACCGAAGTGGACTGTTACTCCAGGGGACAAGCCTTCC
ACCCCCAGCCTGTGTCCAGAGACAGCGCACAGCCAAGTGCGCCAAATGGCCCGGAGCCGGGTGGGAGT
GACCAAGAGCATTCTCCACATCACCAGTGCAGCAGGACTGCCGCTGGGTCCTGCCCCGAGTGCCAAGG
GTCGCTCTACCCCAGTAACCACGATAGAATGCTGACGGCTGTGAAGAAAAAGCCAATGGCATCTCTAG
ATGGGAAAGGGGATTCTTCCTGGACTTTAGCAAGGTTGTATCACCCGGACTCCACAGAGCTACAGCCT
GCATCTTCATTAACTTCAGGCAGTCCAGAGCGCGCGGAAGCCCAGTACTTGCTTGTTTCCAATGGCCA
CCTCCCCAAAGCATGTGACGCCAGTCCCGAGTCCACGCCACTGACAGGACAGCTCCCCGGGAAACAGA
GGGTGCCACTGCTGTTGGCACCAAAAAGCTAGGTTTTGTCTACCTCAGTTCTTGTCATACCAATCTCT
ACGGGAAAGAGAGGTAGGAGAGGCTGCGAGGAAGCTTGGGTTCAAGCGTCACTCATCTGTACATAGTT
GTAACTCCCATGTGGAGTATCAGTCGCTCACAGGACTTGGATCTGAAGCACAGTAAACGCAAGAGGGG
ATTTGTGTACAAAAGGCAAAAAAAGTATTTGATATCATTGTACATAAGAGTTTTCAGAGATTTCATAT
ATATCTTTTACAGAGGCTATTTTAATCTTTAGTGCATGGTTAACAGAAAAAAATTATACAATTTTGAC
AATATTATTTTTCGTATCAGGTTGCTGTTTAATTTTGGAGGGGGTGGGGAAATAGTTCTGGTGCCTTA
ACGCATGGCTGGAATTTATAGAGGCTACAACCACATTTGTTCACAGGAGTTTTTGGTGCGGGGTGGGA
AGGATGGAAGGCCTTGGATTTATATTGCACTTCATAGACCCCTAGGCTGCTGTGCGGTGGGACTCCAC
ATGCGCCGGAAGGAGCTTCAGGTGAGCACTGCTCATGTGTGGATGCCCCTGCAACAGGCTTCCCTGTC
TGTAGAGCCAGGGGTGCAAGTGCCATCCACACTTGCAGTGAATGGCTTTTCCTTTTAGGTTTAAGTCC
TGTCTGTCTGTAAGGCGTAGAATCTGTCCGTCTGTAAGGCGTAGAATGAGGGTTGTTAATCCATCACA
AGCAAAAGGTCAGAACAGTTAAACACTGCCTTTCCTCCTCCTCTTATTTTATGATAAAAGCAAATGTG
GCCTTCTCAGTATCATTCGATTGCTATTTGAGACTTTTAAATTAAGGTAAAGGCTGCTGGTGTTGGTA
CCTGTGGATTTTTCTATACTGATGTTTTCGTTTTGCCAATATAATGAGTATTACATTGGCCTTGGGGG
ACAGAAAGGAGGAAGTTCTGACTTTTCAGGGCTACCTTATTTCTACTAAGGACCCAGAGCAGGCCTGT
CCATGCCATTCCTTCGCACAGATGAAACTGAGCTGGGACTGGAAAGGACAGCCCTTGACCTGGGTTCT
GGGTATAATTTGCACTTTTGAGACTGGTAGCTAACCATCTTATGAGTGCCAATGTGTCATTTAGTAAA
ACTTAAATAGAAACAAGGTCCTTCAAATGTTCCTTTGGCCAAAAGCTGAAGGGAGTTACTGAGAAAAT
AGTTAACAATTACTGTCAGGTGTCATCACTGTTCAAAAGGTAAGCACATTTAGAATTTTGTTCTTGAC
AGTTAACTGACTAATCTTACTTCCACAAAATATGTGAATTTGCTGCTTCTGAGAGGCAATGTGAAAGA
GGGAGTATTACTTTTATGTACAAAGTTATTTATTTATAGAAATTTTGGTACAGTGTACATTGAAAACC
ATGTAAAATATTGAAGTGTCTAACAAATGGCATTGAAGTGTCTTTAATAAAGGTTCATTTAT
AAATGTCA

| | | | |
|---|---|---|---|
| NOV51c, CG59534-02 | SEQ ID NO:1088 | 1093 aa | MW at 119051.7 kD |
| Protein Sequence | | | |

MARPVRGGLGAPRRSPCLLLLWLVLVRLEPVTAAAGPRAPCAAACTCAGDSLDCGGRGLAALPGDLPS
WTRSLNLSYNKLSEIDPAGFEDLPNLQEVYLNNNELTAVPSLGAASSHVVSLFLQHNKIRSVEGSQLK
AYLSLEVLDLSLNNITEVRNTCFPHGPPIKELNLAGNRIGTLELGAFDGLSRSLLTLRLSKNRITQLP
VEAFKLPRLTQLDLNRNRIRLIEGLTFQGLNSLEVLKLQRNNISKLTDGAFWGLSKMHVLHLEYNSLV
EVNSGSLYGLTALHQLHLSNNSIARIHRKGWSFCQKLHELVLSFNNLTRLDEESLAELSSLSVLRLSH
NSISHIAEGAFKGLRSLRVLDLDHNEISGTIEDTSGAFSGLDSLSKLTLFGNKIKSVAKRAFSGLEGL
EHLNLGGNAIRSVQFDAFVKMKNLKELHISSDSFLCDCQLKWLPPWLIGRMLQAFVTATCAHPESLKG
QSIFSVPPESFVCDDFLKPQIITQPETTMAMVGKDIRFTCSAASSSSSPMTFAWKKDNEVLTNADMEN
FVHVHAQDGEVMEYTTILHLRQVTFGHEGRYQCVITNHFGSTYSHKAPLTVNVLPSFTKTPHDITIRT
TTVARLECAATGHPNPQIAWQKDGGTDFPAARERRMHVMPDDDVFFITDVKIDDAGVYSCTAQNSAGS
ISANATLTVLETPSLVVPLEDRVVSVGETVALQCKATGNPPPRITWFKGDRPLSLTERHHLTPDNQLL
VVQNVVAEDAGRYTCEMSNTLGTERAHSQLSVLPAAGCRKDGTTVGIFTIAVVSSIVLTSLVWVCIIY
QTRKKSEEYSVTNTDETVVPPDVPSYLSSQGTLSDRQETVVRTEGGPQANGHIESNGVCPRDASHFPE
PDTHSVACRQPKLCAGSAYHKEPWKAMEKAEGTPGPHKMEHGGRVVCSDCNTEVDCYSRGQAFHPQPV
SRDSAQPSAPNGPEPGGSDQEHSPHHQCSRTAAGSCPECQGSLYPSNHDRMLTAVKKKPMASLDGKGD
SSWTLARLYHPDSTELQPASSLTSGSPERAEAQYLLVSNGHLPKACDASPESTPLTGQLPGKQRVPLL
ILAPKS

| | | |
|---|---|---|
| NOV51d, SNP13377255 of | SEQ ID NO:1089 | 3333 bp |
| CG59534-03, DNA Sequence | ORF Start: ATG at 15 | ORF Stop: TAG at 3219 |
| | SNP Pos: 1912 | SNP Change: T to C |

GCGCTCCAGACAAGATGGCGCGGCCGGTCCGGGGAGGGCTCGGGGCCCCGCGCCGCTCGCCTTGCCTT
CTCCTTCTCTGGCTGGTTTTGGTTCGGCTGGAGCCGGTGACCGCCGCGGCCGGCCCGCGGGCGCCCTG
CGCGGCCGCCTGCACTTGCGCTGGGGACTCGCTGGACTGCGGTGGGCGCGGGCTGGCTGCGTTGCCCG
GGGACCTGCCCTCCTGGACGCGGAGCCTAAACCTGAGTTACAACAAACTCTCTGAGATTGACCCTGCT
GGTTTTGAGGACTTGCCGAACCTACAGGAAGTGCAGCACAACAAGATTCGCAGCGTGGAGGGGAGCCA
GCTGAAGGCCTACCTTTCCTTAGAAGTGTTAGATCTGAGTTTGAACAACATCACGGAAGTGCGGAACA
CCTGCTTTCCACACGGACCGCCTATAAAGGAGCTCAACCTGGCAGGCAATCGGATTGGCACCCTGGAG
TTGGGAGCATTTGATGGTCTGTCACGGTCGCTGCTAACTCTTCGCCTGAGCAAAAACAGGATCACCCA
GCTTCCTGTAAGAGCATTCAAGCTACCCAGGCTGACACAACTGGACCTCAATCGGAACAGGATTCGGC
TGATAGAGGGCCTCACCTTCCAGGGGCTCAACAGCTTGGAGGTGCTGAAGCTTCAGCGAAACAACATC
AGCAAACTGACAGATGGGGCCTTCTGGGGACTGTCCAAGATGCATGTGCTGCACCTGGAGTACAACAG
CCTGGTAGAAGTGAACAGCGGCTCGCTCTACGGCCTCACGGCCCTGCATCAGCTCCACCTCAGCAACA
ATTCCATCGCTCGCATTCACCGCAAGGGCTGGAGCTTCTGCCAGAAGCTGCATGAGTTGGTCCTGTCC
TTCAACAACCTGACACGGCTGGACGAGGAGAGCCTGGCCGAGCTGAGCAGCCTGAGTGTCCTGCGTCT
CAGCCACAATTCCATCAGCCACATTGCGGAGGGTGCCTTCAAGGGACTCAGGAGCCTGCGAGTCTTGG
ATCTGGACCATAACGAGATTTCGGGCACAATAGAGGACACGAGCGGCGCCTTCTCAGGGCTCGACAGC
CTCAGCAAGCTGACTCTGTTTGGAAACAAGATCAAGTCTGTGGCTAAGAGAGCATTCTCGGGGCTGGA
AGGCCTGGAGCACCTGAACCTTGGAGGGAATGCGATCAGATCTGTCCAGTTTGATGCCTTTGTGAAGA
TGAAGAATCTTAAAGAGCTCCATATCAGCAGCGACAGCTTCCTGTGTGACTGCCAGCTGAAGTGGCTG
CCCCCGTGGCTAATTGGCAGGATGCTGCAGGCCTTTGTGACAGCCACCTGTGCCCACCCAGAATCACT
GAAGGGTCAGAGCATTTTCTCTGTGCCACCAGAGAGTTTCGTGTGCGATGACTTCCTGAAGCCACAGA
TCATCACCCAGCCAGAAACCACCATGGCTATGGTGGGCAAGGACATCCGGTTTACATGCTCAGCAGCC
AGCAGCAGCAGCTCCCCCATGACCTTTGCCTGGAAGAAAGACAATGAAGTCCTGACCAATGCAGACAT
GGAGAACTTTGTCCACGTCCACGCGCAGGACGGGGAAGTGATGGAGTACACCACCATCCTGCACCTCC
GTCAGGTCACTTTCGGGCACGAGGGCCGCTACCAATGTGTCATCACCAACCACTTTGGCTCCACCTAT
TCACATAAGGCCAGGCTCACCGTGAATGTGTTGCCATCATTCACCAAAACGCCCCACGACATAACCAT
CCGGACCACCACCGTGGCCCGCCTCGAATGTGCTGCCACAGGTCACCCAAACCCTCAGATTGCCTGGC
AGAAGGATGGAGGCACGGATTTCCCCGCTGCCCGTGAGCGACGCATGCATGTCATGCCGGATGACGAC
GTGTTTTCCATCACTGATGTGAAAATAGATGACGCAGGGGTTTACAGCTGTACTGCTCAGAACTCAGC
CGGTTCTATTTCAGCTAATGCCACCCTGACTGTCCTAGAGACCCCATCCTTGGTGGTCCCCTTGGAAG
ACCGTGTGGTATCTGTGGGAGAAACAGTGGCCCTCCAATGCAAAGCCACGGGGAACCCTCCGCCCCGC
ATCACCTGGTTCAAGGGGGACCGCCCGCTGAGCCTCACTGAGCGGCACCACCTGACCCCTGACAACCA
GCTCCTGGTGGTTCAGAACGTGGTGGCAGAGGATGCGGGCCGATATACCTGTGAGATGTCCAACACCC
TGGGCACGGAGCGAGCTCACAGCCAGCTGAGCGTCCTGCCCGCAGCAGGCTGCAGGAAGGATGGGACC
ACGGTAGGCATCTTCACCATTGCTGTCGTGAGCAGCATCGTCCTGACGTCACTGGTCTGGGTGTGCAT
CATCTACCAGACCAGGAAGAAGAGTGAAGAGTACAGTGTCACCAACACAGATGAAACCGTCGTGCCAC
CAGATGTTCCAAGCTACCTCTCTTCTCAGGGGACCCTTTCTGACCGACAAGAAACCGTGGTCAGGACC
GAGGGTGGCCCTCAGGCCAATGGGCACATTGAGAGCAATGGTGTGTGTCCAAGAGATGCAAGCCACTT
TCCAGAGCCCGACACTCACAGCGTTGCCTGCAGGCAGCCAAAGCTCTGTGCTGGGTCTGCGTATCACA
AAGAGCCGTGGAAAGCGATGGAGAAAGCTGAAGGGACACCTGGGCCACATAAGATGGAACACGGTGGC
CGGGTCGTATGCAGTGACTGCAACACCGAAGTGGACTGTTACTCCAGGGGACAAGCCTTCCACCCCCA
GCCTGTGTCCAGAGACAGCGCACAGCCAAGTGCGCCAAATGGCCCGGAGCCGGGTGGGAGTGACCAAG
AGCATTCTCCACATCACCAGTGCAGCAGGACTGCCGCTGGGTCCTGCCCCGAGTGCCAAGGGTCGCTC
TACCCCAGTAACCACGATAGAATGCTGACGGCTGTGAAGAAAAAGCCAATGGCATCTCTAGATGGGAA
AGGGGATTCTTCCTGGACTTTAGCAAGGTTGTATCACCCGGACTCCACAGAGCTACAGCCTGCATCTT
CATTAACTTCAGGCAGTCCAGAGCGCGCGGAAGCCCAGTACTTGCTTGTTTCCAATGGCCACCTCCCC
AAAGCATGTGACGCCAGTCCCGAGTCCACGCCACTGACAGGACAGCTCCCCGGGAAACAGAGGGTGCC
ACTGCTGTTGGCACCAAAAAGCTAGGTTTTGTCTACCTCAGTTCTTGTCATACCAATCTCTACGGGAA
AGAGAGGTAGGAGAGGCTGCGAGGAAGCTTGGGTTCAAGCGTCACTCATCTGTACATAGTTGT
AACTCC

| | | | |
|---|---|---|---|
| NOV51d, SNP13377255 of | SEQ ID NO:1090 | 1068 aa | MW at 116392.7 kD |
| CG59534-03, Protein Sequence | SNP Pos: 633 | | SNP Change: Phe to Ser |

MARPVRGGLGAPRRSPCLLLLWLVLVRLEPVTAAAGPRAPCAAACTCAGDSLDCGGRGLAALPGDLPS
WTRSLNLSYNKLSEIDPAGFEDLPNLQEVQHNKIRSVEGSQLKAYLSLEVLDLSLNNITEVRNTCFPH
GPPIKELNLAGNRIGTLELGAFDGLSRSLLTLRLSKNRITQLPVRAFKLPRLTQLDLNRNRIRLIEGL
TFQGLNSLEVLKLQRNNISKLTDGAFWGLSKMHVLHLEYNSLVEVNSGSLYGLTALHQLHLSNNSIAR
IHRKGWSFCQKLHELVLSFNNLTRLDEESLAELSSLSVLRLSHNSISHIAEGAFKGLRSLRVLDLDHN
EISGTIEDTSGAFSGLDSLSKLTLFGNKIKSVAKRAFSGLEGLEHLNLGGNAIRSVQFDAFVKMKNLK
ELHISSDSFLCDCQLKWLPPWLIGRMLQAFVTATCAHPESLKGQSIFSVPPESFVCDDFLKPQIITQP
ETTMAMVGKDIRFTCSAASSSSSPMTFAWKKDNEVLTNADMENFVHVHAQDGEVMEYTTILHLRQVTF
GHEGRYQCVITNHFGSTYSHKARLTVNVLPSFTKTPHDITIRTTTVARLECAATGHPNPQIAWQKDGG
TDFPAARERRMHVMPDDDVFSITDVKIDDAGVYSCTAQNSAGSISANATLTVLETPSLVVPLEDRVVS
VGETVALQCKATGNPPPRITWFKGDRPLSLTERHHLTPDNQLLVVQNVVAEDAGRYTCEMSNTLGTER
AHSQLSVLPAAGCRKDGTTVGIFTIAVVSSIVLTSLVWVCIIYQTRKKSEEYSVTNTDETVVPPDVPS
YLSSQGTLSDRQETVVRTEGGPQANGHIESNGVCPRDASHFPEPDTHSVACRQPKLCAGSAYHKEPWK
AMEKAEGTPGPHKMEHGGRVVCSDCNTEVDCYSRGQAFHPQPVSRDSAQPSAPNGPEPGGSDQEHSPH
HQCSRTAAGSCPECQGSLYPSNHDRMLTAVKKKPMASLDGKGDSSWTLARLYHPDSTELQPASSLTSG
SPERAEAQYLLVSNGHLPKACDASPESTPLTGQLPGKQRVPLLLAPKS

A ClustalW comparison of the above protein sequences yields the following sequence alignment shown in Table 51B.

TABLE 51B
Comparison of the NOV51 protein sequences.

| | | |
|---|---|---|
| NOV51a | MARPVRGGLGAPRRSPCLLLLWLVLVRLEPVTAAAGPRAPCAAACTCAGDSLDCGGRGLA | (SEQ ID NO:1084) |
| NOV51b | ----MRWDLAVLPR--------LVLISWAQVILLPWP----AAACTCAGDSLDCGGRGLA | (SEQ ID NO:1086) |
| NOV51c | MARPVRGGLGAPRRSPCLLLLWLVLVRLEPVTAAAGPRAPCAAACTCAGDSLDCGGRGLA | (SEQ ID NO:1088) |
| NOV51a | ALPGDLPSWTRSLNLSYNKLSEIDPAGFEDLPNLGEV----------------------- | |
| NOV51b | ALPGDLPSWTRSLNLSYNKLSEIDPAGFEDLPNLQEVYLNNNELTAVPSLGAASSHVVSL | |
| NOV51c | ALPGDLPSWTRSLNLSYNKLSEIDPAGFEDLPNLQEVYLNNNELTAVPSLGAASSHVVSL | |
| NOV51a | --QHNKIRSVEGSQLKAYLSLEVLDLSLNNITEVRNTCFPHGPPIKELNLAGNRIGTLEL | |
| NOV51b | FLQHNKIRSVEGSQLKAYLSLEVLDLSLNNITEVRNTCFPHGPPIKELNLAGNRIGTLEL | |
| NOV51c | FLQHNKIRSVEGSQLKAYLSLEVLDLSLNNTTEVRNTCFPHGPPIKELNLAGNRIGTLEL | |
| NOV51a | GAFDGLSRSLLTLRLSKNRITQLPVRAFKLPRLTQLDLNPNRIRLIEGLTFQGLNSLEVL | |
| NOV51b | GAFDGLSRSLLTLRLSKNRITQLPVRAFKLPRLTQLDLNRNRIRLIEGLTFQGLNSLEVL | |
| NOV51c | GAFDGLSRSLLTLRLSKNRITQLPVRAFKLPRLTQLDLNRNRIRLIEGLTFQGLNSLEVL | |
| NOV51a | KLQRNNISKLTDGAFWGLSKMHVLHLEYNSLVEVNSGSLYGLTALHQLHLSNNSIARIHR | |
| NOV51b | KLQRNNISKLTDGAFWGLSKMHVLHLEYNSLVEVNSGSLYGLTALHQLHLSNNSIARIHR | |
| NOV51c | KLQRNNISKLTDGAFWGLSKMHVLHLEYNSLVEVNSGSLYGLTALHQLHLSNNSIARIHR | |
| NOV51a | KGWSFCQKLHELVLSFNNLTRLDEESIAELSSLSVLRLSHNSISHIAEGAFKGLRSLRVL | |
| NOV51b | KGWSFCQKLHELVLSFNNLTRLDEESLAELSSLSVLRLSHNSISHIAEGAFKGLRSLRVL | |
| NOV51c | KGWSFCQKLHELVLSFNNLTRLDEESLAELSSLSVLRLSHNSISHIAEGAFKGLRSLRVL | |
| NOV51a | DLDHNEISGTIEDTSGAFSGLDSLSKLTLFGNKIKSVAKRAFSGLEGLEHLNLGGNAIRS | |
| NOV51b | DLDHNEISGTIEDTSGAFSGLDSLSKLTLFGNKIKSVAKRAFSGLEGLEHLNLGGNAIRS | |
| NOV51c | DLDHNEISGTIEDTSGAFSGLDSLSKLTLFGNKIKSVAKRAFSGLEGLEHLNLGGNAIRS | |
| NOV51a | VQFDAFVKMKNLKELHISSDSFLCDCQLKWLPPWLIGRMLQAFVTATCAHPESLKGQSIF | |
| NOV51b | VQFDAFVKMKNLKELHISSDSFLCDCQLKWLPPWLIGRMLQAFVTATCAHPESLKGQSIF | |
| NOV51c | VQFDAFVKMKNLKELHISSDSFLCDCQLKWLPPWLIGRMLQAFVTATCAHPESLKGQSIF | |
| NOV51a | SVPPESFVCDDFLKPQIITQPETTMAMVGKDIRFTCSAASSSSSPMTFAWKKDNEVLTNA | |
| NOV51b | SVPPESFVCDDFLKPQIITQPETTMAMVGKDIRFTCSAASSSSSPMTFAWKKDNEVLTNA | |
| NOV51c | SVPPESFVCDDFLKPQIITQPETTMAMVGKDIRFTCSAASSSSSPMTFAWKKDNEVLTNA | |
| NOV51a | DMENFVNVHAQDGEVMEYTTILHLRQVTFGHEGRYQCVITNHFGSTYSHKARLTVNVLPS | |
| NOV51b | DMENFVHVHAQDGEVMEYTTILHLRQVTFGHEGRYQCVITNHFGSTYSHKARLTVNVLPS | |
| NOV51c | DMENFVHVHAQDGEVMEYTTILHLRQVTFGHEGRYQCVITNHFGSTYSHKARLTVNVLPS | |
| NOV51a | FTKTPHDITIRTTTVARLECAATGHPNPQIAWQKDGGTDFPAARERRMHVMPDDDVFFIT | |
| NOV51b | FTKTPHDITIRTTTMARLECAATGHPNPQIAWQKDGGTDFPAARERRMHVMPDDDVFFIT | |
| NOV51c | FTKTPHDITIRTTTVARLECAATGHPNPQIAWQKDGGTDFPAARERRMHVMPDDDVFFIT | |
| NOV51a | DVKIDDAGVYSCTAQNSAGSISANATLTVLETPSLVVPLEDRVVSVGETVALQCKATGNP | |
| NOV51b | DVKIDDAGVYSCTAQNSAGSISANATLTVLETPSLVVPLEDRVVSVGETVALQCKATGNP | |
| NOV51c | DVKIDDAGVYSCTAQNSAGSISANATLTVLETPSLVVPLEDRVVSVGETVALQCKATGNP | |
| NOV51a | PPRITWFKGDRPLSLTERHHLTPDNQLLVVQNVVAEDAGRYTCEMSNTLGTERAHSQLSV | |
| NOV51b | PPRITWFKGDRPLSLTERHHLTPDNQLLVVQNVVAEDAGRYTCEMSNTLGTERAHSQLSV | |
| NOV51c | PPRITWFKGDRPLSLTERHHLTPDNQLLVVQNVVAEDAGRYTCEMSNTLGTERAHSQLSV | |
| NOV51a | LPAAGCRKDGTTVGIFTIAVVSSIVLTSLVWVCIIYQTRKKSEEYSVTNTDETVVPPDVP | |
| NOV51b | LPAAGCRKDGTTVGIFTIAVVSSIVLTSLVWVCIIYQTRKKSEEYSVTNTDETVVPPDVP | |
| NOV51c | LPAAGCRKDGTTVGIFTIAVVSSIVLTSLVWVCIIYQTRKKSEEYSVTNTDETVVPPDVP | |
| NOV51a | SYLSSQGTLSDRQETVVRTEGGPQANGHIESNGVCPRDASHFPEPDTHSVACRQPKLCAG | |
| NOV51b | SYLSSQGTLSDRQETVVRTEGGPQANGHIESNGVCPRDASHFPEPDTHSVACRQPKLCAG | |
| NOV51c | SYLSSQGTLSDRQETVVRTEGGPQANGHIESNGVCPRDASHFPEPDTHSVACRQPKLCAG | |
| NOV51a | SAYHKEPWKAMEKAEGTPGPHKMEHGGRVVCSDCNTEVDCYSRGQAFHPQPVSRDSAQPS | |
| NOV51b | SAYHKEPWKAMEKAEGTPGPHKMEHGGRVVCSDCNTEVDCYSRGQAFHPQPVSRDSAQPS | |
| NOV51c | SAYHKEPWKAMEKAEGTPGPHKMEHGGRVVCSDCNTEVDCYSRGQAFHPQPVSRDSAQPS | |
| NOV51a | APNGPEPGGSDQEHSPHHQCSRTAAGSCPECQGSLYPSNHDRMLTAVKKKPMASLDGKGD | |
| NOV51b | APNGPEPGGSDQEHSPHHQCSRTAAGSCPECQGSLYPSNHDRMLTAVKKKPMASLDGKGD | |
| NOV51c | APNGPEPGGSDQEHSPHHQCSRTAAGSCPECQGSLYPSNHDRMLTAVKKKPMASLDGKGD | |
| NOV51a | SSWTLARLYHPDSTELQPASSLTSGSPERAEAQYLLVSNGHLPKACDASPESTPLTGQLP | |
| NOV51b | SSWTLARLYHPDSTELQPASSLTSGSPERAEAQYLLVSNGHLPKACDASPESTPLTGQLP | |
| NOV51c | SSWTLARLYHPDSTELQPASSLTSGSPERAEAQYLLVSNGHLPKACDASPESTPLTGQLP | |
| NOV51a | GKQRVPLLIAPKS | |
| NOV51b | GKQRVPLLLAPKS | |
| NOV51c | GKQRVPLLLAPKS | |

Further analysis of the NOV51a protein yielded the following properties shown in Table 51C.

TABLE 51C
Protein Sequence Properties NOV51a

| | |
|---|---|
| SignalP analysis: | Cleavage site between residues 36 and 37 |
| PSORT II analysis: | |

| PSG: | a new signal peptide prediction method | |
|---|---|---|
| | N-region: | length 6; pos.chg 2; neg.chg 0 |
| | H-region: | length 6; peak value −9.10 |
| | PSG score: | −13.50 |

GvH: von Heijne's method for signal seq. recognition
GvH score (threshold: −2.1): −0.07
possible cleavage site: between 33 and 34

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 2 | | |
| | INTEGRAL | Likelihood = | Transmembrane 17-33 |
| | | −2.87 | |
| | INTEGRAL | Likelihood = | Transmembrane 773-789 |
| | | −8.23 | |

| | |
|---|---|
| PERIPHERAL | Likelihood = 3.76 (at 656) |

| | |
|---|---|
| | ALOM score: −8.23 (number of TMSs: 2) |
| MTOP: | Prediction of membrane topology (Hartmann et al.) |
| | Center position for calculation: 24 |
| | Charge difference: −4.0 C(1.0)-N(5.0) |
| | N >= C: N-terminal side will be inside |

>>> membrane topology: type 3a

| MITDISC: | discrimination of mitochondrial targeting seq | | | |
|---|---|---|---|---|
| | R content: | 5 | Hyd Moment(75): | 4.80 |
| | Hyd Moment(95): | 12.52 | G content: | 3 |
| | D/E content: | 1 | S/T content: | 1 |

Score: −0.99
Gavel: prediction of cleavage sites for mitochondrial preseq
R-2 motif at 48 PRA|PC

NUCDISC: discrimination of nuclear localization signals

pat4: KKKP (4) at 983
pat7: PAARERR (3) at 616
bipartite: none
content of basic residues: 9.7%
NLS Score: 0.04
KDEL: ER retention motif in the C-terminus: none

ER Membrane Retention Signals:

XXRR-like motif in the N-terminus: ARPV
KKXX-like motif in the C-terminus: LAPK

SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs:

Leucine zipper pattern (PS00029): *** found ***
LDCGGRGLAALPGDLPSWTRSL at 52

none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 70.6 |
| COIL: | Lupas's algorithm to detect coiled-coil regions |
| | total: 0 residues |

Final Results (k = 9/23):

```
34.8%: nuclear
30.4%: mitochondrial
21.7%: endoplasmic reticulum
  4.3%: plasma membrane
  4.3%: cytoplasmic
  4.3%: peroxisomal
```

>> prediction for CG59534-03 is nuc (k = 23)

A search of the NOV51a protein against the Geneseq database, a proprietary database that contains sequences published in patents and patent publication, yielded several homologous proteins shown in Table 51D.

TABLE 51D
Geneseq Results for NOV51a

| | | NOV51a | Identities/ | |
|---|---|---|---|---|
| Geneseq | Protein/Organism/Length | Residues/Match | Similarities for the | Expect |
| Identifier | [Patent #, Date] | Residues | Matched Region | Value |
| ABG69660 | Human secreted protein SCEP-40 - | 1 . . . 1068 | 1065/1093 (97%) | 0.0 |
| | Homo sapiens, 1093 aa. | 1 . . . 1093 | 1068/1093 (97%) | |
| | [WO200248337-A2, 20-JUN-2002] | | | |
| AAY08008 | Human HLIG-1 protein - Homo sapiens, | 1 . . . 1068 | 1053/1101 (95%) | 0.0 |
| | 1101 aa. [EP913472-A2, | 1 . . . 1101 | 1062/1101 (95%) | |
| | 06-MAY-1999] | | | |
| ABG69674 | Human secreted protein SCEP-54 - | 1 . . . 1068 | 1042/1093 (95%) | 0.0 |
| | Homo sapiens, 1070 aa. | 1 . . . 1070 | 1045/1093 (95%) | |
| | [WO200248337-A2, 20-JUN-2002] | | | |
| ABP70116 | Human NOV30a - Homo sapiens, | 42 . . . 1068 | 1026/1052 (97%) | 0.0 |
| | 1077 aa. [WO200272771-A2, | 26 . . . 1077 | 1027/1052 (97%) | |
| | 19-SEP-2002] | | | |
| AAY97833 | Murine LIG-1 protein - Mus sp, 1091 | 1 . . . 1068 | 887/1102 (80%) | 0.0 |
| | aa. [JP2000093043-A, 04-APR-2000] | 1 . . . 1091 | 945/1102 (85%) | |

In a BLAST search of public sequence databases, the NOV51 a protein was found to have homology to the proteins shown in the BLASTP data in Table 51E.

TABLE 51E
Public BLASTP Results for NOV51a

| Protein | | NOV51a | Identities/ | | |
|---|---|---|---|---|---|
| Accession | | Residues/Match | Similarities for the | | Expect |
| Number | Protein/Organism/Length | Residues | Matched Portion | | Value |
| Q96JA1 | Membrane glycoprotein LIG-1 - | 1 . . . 1068 | 1068/1093 | (97%) | 0.0 |
| | Homo sapiens (Human), 1093 aa. | 1 . . . 1093 | 1068/1093 | (97%) | |
| Q9BYB8 | Membrane glycoprotein LIG-1 - | 1 . . . 1068 | 1064/1094 | (97%) | 0.0 |
| | Homo sapiens (Human), 1094 aa. | 1 . . . 1094 | 1068/1094 | (97%) | |
| P70193 | Membrane glycoprotein - | 1 . . . 1068 | 887/1102 | (80%) | 0.0 |
| | Mus musculus (Mouse), 1091 aa. | 1 . . . 1091 | 945/1102 | (85%) | |
| Q9UFI4 | Hypothetical protein - | 586 . . . 1068 | 481/483 | (99%) | 0.0 |
| | Homo sapiens (Human), 483 aa | 1 . . . 483 | 481/483 | (99%) | |
| | (fragment). | | | | |
| O94898 | Hypothetical protein KIAA0806 - | 41 . . . 993 | 491/1002 | (49%) | 0.0 |
| | Homo sapiens (Human), 1065 aa. | 43 . . . 991 | 624/1002 | (62%) | |

PFam analysis predicts that the NOV51a protein contains the domains shown in the Table 51F.

TABLE 51F
Domain Analysis of NOV51a

| | | | |
|---|---|---|---|
| | | Identities/ | |
| | | Similarities | |
| Pfam | NOV51a | for the | Expect |
| Domain | Match Region | Matched Region | Value |

| | | | | |
|---|---|---|---|---|
| LRRNT | 40 . . . 67 | 17/35 | (49%) | 0.0012 |
| | | 20/35 | (57%) | |
| LRR | 211 . . . 234 | 10/25 | (40%) | 0.063 |
| | | 21/25 | (84%) | |
| LRR | 283 . . . 306 | 11/25 | (44%) | 0.16 |
| | | 20/25 | (80%) | |
| LRR | 307 . . . 330 | 9/25 | (36%) | 0.0016 |
| | | 23/25 | (92%) | |
| LRR | 382 . . . 405 | 9/25 | (36%) | 0.18 |
| | | 18/25 | (72%) | |
| LRRCT | 415 . . . 465 | 22/54 | (41%) | 1.6e−13 |
| | | 42/54 | (78%) | |
| ig | 484 . . . 554 | 13/72 | (18%) | 2e−05 |
| | | 50/72 | (69%) | |
| ig | 588 . . . 649 | 13/65 | (20%) | 0.025 |
| | | 43/65 | (66%) | |
| ig | 682 . . . 740 | 19/62 | (31%) | 1.4e−08 |
| | | 45/62 | (73%) | |

### Example 52

The NOV52 clone was analyzed, and the nucleotide and encoded polypeptide sequences are shown in Table 52A.

TABLE 52A
NOV52 Sequence Analysis

| | | |
|---|---|---|
| NOV52a, CG59584-03 | SEQ ID NO:1091 | 4994 bp |
| DNA Sequence | ORF Start: ATG at 36 | ORF Stop: TGA at 4815 |

CCTGGACCTAATACCCCTTCTTCCTAAAGCACAAAATGATTAAATTGCTTAATCCAGACAACTCTCCA
ATCCCAAATGAAGTTGTTCAGTTGCATCTGAAGGACAAAATCGTGGGAAACTACACCACAGATGTAAA
TGGCATCGCTCAGTTTTTCTTGGACACATATACGTTTACATACCCAAATATCACTTTGAAAGCAGCCT
ACAAGGCCAATGAAAATTGCCAGGCTCATGGCTGGGTGTTGCCTCAATACCCTCAGCCCGAGTACTTT
GCATATCGATTTTACTCCAAGATGAATAGCTTCCTAAAGATTGTCCAAGAGATGGAAGAACTGAGATG
CAACCAGCAGAAGAGGGTGCTAGTGCACTGCATTCTCAATATGGAAGACTTTGAAGACAAAACCTACA
CAGCAGACTTCAATTATTTGGTGATTTCAAAAGGTGTAATCATTCTTCATGGGCAACAGAAAATTGAG
ATCAACGAAAATGGGAGGAAGGGCATATTTTCCATTTCTATAGACATTAACCCTGAATTAGCGCCCTC
AGTAGATATGCTTGTCTATAGCTTGCATCCTGGAGGAGAAATGGTCACTGATAGCACCCAATTCCGAA
TTGAGAAATGCTTCGAAAATCAGGTCAACTTAAATTTTTCTAAAGAAAAAAGTTTACCAGGATCCAAT
ATTGATCTTCAAGTCTCGGCTGCTTCAAACTCTCTTTGTGCTCTTTGGGCTGTAGACCAGAGTGTATT
GCTACTAAGGAATTATGGTCAGCTGTCAGCACAAACTGTGTATAGTCAGCTATATTCCAGGGAACTAC
ATGGCTATTACTTCAGAGGACTTAACTTAGAAGATGGCCTTAAAGTGCCGTGTCTTGAAGATGAACAT
ATCCTTTACAATGGAATTTATTACACACCTGCATGGGCTGACTTTGGAAAAGATGGCTATGACCTTGT
GAAGGATCCTCAAAACAATCGGATTTTTCAAAGGCAAAATGTGACTTCTTTCCGAAATATTACCCAAC
TCTCGTTCCAACTGATTTCAGAACCAATGTTTGGAGATTACTGGATTGTTGTGAAAAGAAACTCAAGG
GAGACAGTGACACACCAATTTGCTGTTAAAAGATATGTGCTGCCCAAGTTTGAAGTTACAGTCAATGC
ACCACAAACAGTAACTATTTCAGATGATGAATTCCAAGTGGATGTATGTGCTAAGTACAACTTTGGCC
AACCTGTGCAAGGGGAAACCCAAATCCGGGTGTGCAGAGAGTATTTTTCTTCAAGCAATTGTGAGAAA
AATGAAAATGAAATATGTGAGCAATTTATTGCACAGTTGGAAAATGGTTGTGTTTCTCAAATTGTAAA
TACAAAAGTCTTCCAACTCTACCGTTCGGGATTGTTCATGACATTTCATGTCGCTGTAATTGTTACAG
AATCTGGGACAGTTATGCAGATCAGCGAGAAGACCTCAGTTTTTATCACTCAATTGCTTGGAACTGTA
AACTTTGAGAACATGGATACATTCTATAGAAGAGGGATTTCTTATTTTGGAACTCTTAAATTTTCGGA
TCCCAATAATGTACCTATGGTGAACAAGTTGTTGCAACTGGAGCTCAATGATGAATTTATAGGAAATT
ACACTACGGATGAGAATGGCGAAGCTCAATTTTCCATTGACACTTCAGACATATTTGATCCAGAGTTC
AACCTAAAAGCCACATATGTTCGACCTGAGAGCTGCTATCTTCCCAGCTGGTTGACGCCTCAGTACTT
GGATGCTCACTTCTTAGTCTCACGCTTTTACTCCCGAACCAACAGCTTCCTGAAGATTGTTCCAGAAC
CAAAGCAGCTTGAATGTAATCAACAGAAGGTTGTTACTGTGCATTACTCCCTAAACAGTGAAGCATAT
GAGGATGATTCCAATGTAAAGTTCTTCTATTTGATGATGGTAAAAGGAGCTATCTTACTCAGTGGACA
AAAGGAAATCAGAAACAAAGCCTGGAATGGAAACTTCTCGTTCCCAATCAGCATCAGTGCTGATCTGG
CTCCTGCAGCCGTCCTGTTTGTCTATACCCTTCACCCCAGTGGGGAAATTGTGGCTGACAGTGTCAGA
TTCCAGGTTGACAAGTGCTTTAAACACAAGGTTAACATAAAGTTCTCTAACGAGCAGGGCTTACCTGG
TTCCAATGCTAGTCTCTGTCTTCAAGCGGCGCCTGTCTTATTCTGTGCCCTCAGGGCTGTGGATAGGA
ATGTCCTTCTACTGAAATCTGAACAACAGCTGTCAGCTGAAAGTGTGTATAACATGGTTCCAAGTATA
GAGCCGTATGGTTATTTCTACCATGGCCTCAATCTTGATGATGGCAAGGAAGACCCTTGCATTCCTCA
GAGGGATATGTTCTACAATGGTTTATATTACACACCTGTAAGCAACTATGGGGATGGAGATATCTATA
ATATTGTCAGGAACATGGGTCTAAAAGTCTTTACCAATCTCCATTACCGAAAACCAGAAAAAATTATG
GTCCAATGCGTAGTGTTCCGTCTAGAATTGCATGTAGCTAGTGGAATCAGAGGGGAGAATGCTGACTA
TGTAGAACAGGCTATAATTCAAACAGTAAGAACAAACTTCCCAGAGACATGGATGTGGGACCTCGTCA
GTGTCGATTCCTCAGGCTCTGCCAATCTTTCGTTCCTCATTCCTGATACGATAACCCAATGGGAGGCA
AGTGGCTTTTGTGTGAATGGTGACGTTGGATTTGGCATTTCCTCTACAACCACTCTAGAAGTCTCCCA
ACCTTTCTTTATTGAGATTGCCTCACCCTTTTCGGTTGTTCAAAATGAACAATTTGATTTGATTGTCA
ATGTCTTCAGCTACCGGAATACATGTGTAGAGATTTCTGTTCAAGTGGAGGAGTCTCAGAATTATGAA
GCAAATATTCATACCTTGAAAATCAATGGCAGTGAGGTTATTCAAGCTGGAGGGAGGAAAACAAACGT
CTGGACTATTATACCTAAGAAATTGGGTAAAGTGAATATCACTGTAGTTGCTGAGTCCAAACAAAGCA
TTAGTAGAGCCTGAAGGTATTGAAAAGGAAAGGACCCAGAGTTTCCTTATCTGTACAGAAGGTGCCAA
CTGTTGTGGGGGATATTCTAGGACTTGCCTTGCAGAATCTGGTTGTTCTCCAAATGCCCTATGGAAGT
GGAGAGCAGAATGCTGCCCTACTAGCATCTGATACTTATGTTCTGGACTATCTGAAATCTACTGAGCA
ACTGACAGAGGAAGTTCAATCTAAGGCTTTCTTTCTCTTATCTAATGGTTATCAAAGGCAATTATCTT
TCAAAAACTCTGATGGTTCCTATAGTGTGTTTTGGCAGCAGAGTCAGAAAGGAAGCATATGGCTCAGT
GCTCTTACTTTTAAGACATTGGAGAGAATGAAAAAATATGTATTCATTGATGAAAATGTTCAAAAACA
GACCTTAATCTGGCTTTCAAGCCAACAGAAAACAAGCGGCTGCTTTAAGAATGATGGCCAGCTTTTCA
ACCACGCCTGGGAGGGTGGAGATGAAGAGGACATTTCACTCACTGCGTATGTTGTTGGGATGTTCTTT
GAAGCTGGGCTCAATTTCACTTTTCCTGCTCTACGAAACGCACTCTTTTGCCTTGAAGCGGCATTGGA
CAGTGGTGTCACTAATGGCTATAATCATGCAATTCTAGCTTATGCTTTTGCCTTAGCTGGAAAAGAGA
AGCAAGTGGAATCTTTACTCCAAACCCTGGATCAATCTGCCCCAAAACTAAATAATGTCATCTACTGG
TGAGAAGAGTTGCTACGTGCTGTTGGCTGTCATTTCCCGGAAAATTCCTGACCTCACCTATGCTAGTA
AGATTGTGCAGTGGCTTGCCCAACGGATGAATTCCCATGGAGGCTTTTCTTCCAACCAGACACCTGAT
GATACTCTGTTCAAATTATATACGGGCCAAAAAGAAAGCTTTCGCTCTAGTTCTGTGGGCTATACACT
GGGAAAAGCAAATGAAAAGAAGGAAAACAGGAGAAATGGGGGTGAAGGATCCAGTGAGATTTTCCAGG
TTAACGGTCATAACCGCCTACTGGTCCAACGTTCAGAAGTAACACAGGCACCTGGAGAATACACAGTA
GATGTGGAAGGACACGGTTGTACATTTATCCAGGCCACCCTTAAGTACAATGTTCTCCTACCTAAGAA
GGCATCTGGATTTTCTCTTTCCTTGGAAATAGTAAAGAACTACTCTTCGACTGCTTTTGACCTCACAG
TGACCCTCAAATACACTGGAATTCGCAATAAATCCAGTATGGTGGTTATAGATGTAAAAATGCTATCA
CAAGAATGACCATGTTCTTTTCTACTTGGAAAATGTAGGTTTTGGTCGAGCAGACAGTTTCCCTTTTT
CTGTTGAGCAGAGCAACCTTGTGTTCAACATTCAGCCAGCCCCAGCCATGGTCTACGATTATTATGAA
AAAGAAGAATATGCCCTAGCTTTTTACAACATCGACAGTAGTTCAGTTTCCGAGTGAGACAAAGCAAT
TACTAGAAGAGTTGGAGAAGCATTTCTTGTAACAAACTGATTCTTCTGTATCAAACCTGGAAAAAAAT
CATGAACCATCTGACATCGTGAACAGTCTGCAGTGGGCTATGGTTTCTTGTCAAGTCTTATTTCCTTA
TCATCCCATTAAATGTTGTCATTTTGCAAA

| | | | |
|---|---|---|---|
| NOV52a CG59584-03 | SEQ ID NO:1092 | 1593 aa | MW at 180167.3 kD |
| Protein | | | |

MIKLLNPDNSPIPNEVVQLHLKDKIVGNYTTDVNGIAQFFLDTYTFTYPNITLKAAYKANENCQAHGW
VLPQYPQPEYFAYRFYSKMNSFLKIVQEMEELRCNQQKRVLVHCILNMEDFEDKTYTADFNYLVISKG
VIILHGQQKIEINENGRKGIFSISIDINPELAPSVDMLVYSLHPGGEMVTDSTQFRIEKCFENQVNLN
FSKEKSLPGSNIDLQVSAASNSLCALWAVDQSVLLLRNYGQLSAQTVYSQLYSRELHGYYFRGLNLED
GLKVPCLEDEHILYNGIYYTPAWADFGKDGYDLVKDPQNNRIFQRQNVTSFRNITQLSFQLISEPMFG
DYWIVVKRNSRETVTHQFAVKRYVLPKFEVTVNAPQTVTISDDEFQVDVCAKYNFGQPVQGETQIRVC
REYFSSSNCEKNENEICEQFIAQLENGCVSQIVNTKVFQLYRSGLFMTFHVAVIVTESGTVMQISEKT
SVFITQLLGTVNFENMDTFYRRGISYFGTLKFSDPNNVPMVNKLLQLELNDEFIGNYTTDENGEAQFS
IDTSDIFDPEFNLKATYVRPESCYLPSWLTPQYLDAHFLVSRFYSRTNSFLKIVPEPKQLECNQQKVV
TVHYSLNSEAYEDDSNVKFFYLMMVKGAILLSGQKEIRNKAWNGNFSFPISISADLAPAAVLFVYTLH
PSGEIVADSVRFQVDKCFKHKVNIKFSNEQGLPGSNASLCLQAAPVLFCALRAVDRNVLLLKSEQQLS
AESVYNMVPSIEPYGYFYHGLNLDDGKEDPCIPQRDMFYNGLYYTPVSNYGDGDIYNIVPNMGLKVFT
NLHYRKPEKIMVQCVVFRLELHVASGIRGENADYVEQAIIQTVRTNFPETWMWDLVSVDSSGSANLSF
LIPDTITQWEASGFCVNGDVGFGISSTTTLEVSQPFFIEIASPFSVVQNEQFDLIVNVFSYRNTCVEI
SVQVEESQNYEANIHTLKINGSEVIQAGGRKTNVWTIIPKKLGKVNITVVAESKQSSACPNEGMEQQK
LNWKDTVVQSFLVEPEGIEKERTQSFLICTEGAKASKQGVLDLPNDVVEGSARGFFTVVGDILGLALQ
NLVVLQMPYGSGEQNAALLASDTYVLDYLKSTEQLTEEVQSKAFFLLSNGYQRQLSFKNSDGSYSVFW
QQSQKGSIWLSALTFKTLERMKKYVFIDENVQKQTLIWLSSQQKTSGCFKNDGQLFNHAWEGGDEEDI
SLTAYVVGMFFEAGLNFTFPALRNALFCLEAALDSGVTNGYNHAILAYAFALAGKEKQVESLLQTLDQ
SAPKLNNVIYWERERKPKTEEFPSFIPWAPSAQTEKSCYVLLAVISRKIPDLTYASKIVQWLAQRMNS
HGGFSSNQTPDDTLFKLYTGQKESFRSSSVGYTLGKANEKKENRRNGGEGSSEIFQVNGHNRLLVQRS
EVTQAPGEYTVDVEGHGCTFIQATLKYNVLLPKKASGFSLSLEIVKNYSSTAFDLTVTLKYTGIRNKS
SMVVIDVKMLSGFTPTMSSIEELENKGQVMKTEVKNDHVLFYLENVGFGRADSFPFSVEQSNLVFNIQ
PAPAMVYDYYEKEEYALAFYNIDSSSVSE

| | | |
|---|---|---|
| NOV52b, CG59584-02 | SEQ ID NO:1093 | 4501 bp |
| DNA Sequence | ORF Start: at 1 | ORF Stop: TGA at 4309 |

TCCATTTCTATAGACATTAACCCTGAATTAGCGCCCTCAGTAGATATGCTTGTCTATAGCTTGCATCC
TGGAGGAGAAATGGTCACTGATAGCACCCAATTCCGAATTGAGAAATGCTTCGAAAATCAGGTCAACT
TAAATTTTTCTAAAGAAAAAAGTTTACCAGGATCCAATATTGATCTTCAAGTCTCGGCTGCTTCAAAC
TCTCTTTGTGCTCTTTGGGCTGTAGACCAGAGTGTATTGCTACTAAGGAATTATGGTCAGCTGTCAGC
ACAAACTGTGTATAGTCAGCTATATTCCAGGGAACTACATGGCTATTACTTCAGAGGACTTAACTTAG
AAGATGGCCTTAAAGTGCCGTGTCTTGAAGATGAACATATCCTTTACAATGGAATTTATTACACACCT
GCATGGGCTGACTTTGGAAAAGATGGCTATGACCTTGTGAAGGATCCTCAAAACAATCGGATTTTTCA
AAGGCAAAATGTGACTTCTTTCCGAAATATTACCCAACTCTCGTTCCAACTGATTTCAGAACCAATGT
TTGGAGATTACTGGATTGTTGTGAAAAGAAACTCAAGGGAGACAGTGACACACCAATTTGCTGTTAAA
AGATATGTGCTGCCCAAGTTTGAAGTTACAGTCAATGCACCACAAACAGTAACTATTTCAGATGATGA
ATTCCAAGTGGATGTATGTGCTAAGTACAACTTTGGCCAACCTGTGCAAGGGGAAACCCAAATCCGGG
TGTGCAGAGAGTATTTTTCTTCAAGCAATTGTGAGAAAAATGAAAATGAAATATGTGAGCAATTTATT
GCACAGTTGGAAAATGGTTGTGTTTCTCAAATTGTAAATACAAAAGTCTTCCAACTCTACCGTTCGGG
ATTGTTCATGACATTTCATGTCGCTGTAATTGTTACAGAATCTGGGACAGTTATGCAGATCAGCGAGA
AGACCTCAGTTTTTATCACTCAATTGCTTGGAACTGTAAACTTTGAGAACATGGATACATTCTATAGA
AGAGGGATTTCTTATTTTGGAACTCTTAAATTTTCGGATCCCAATAATGTACCTATGGTGAACAAGTT
GTTGCAACTGGAGCTCAATGATGAATTTATAGGAAATTACACTACGGATGAGAATGGCGAAGCTCAAT
TTTCCATTGACACTTCAGACATATTTGATCCAGAGTTCAACCTAAAAGCCACATATGTTCGACCTGAG
AGCTGCTATCTTCCCAGCTGGTTGACGCCTCAGTACTTGGATGCTCACTTCTTAGTCTCACGCTTTTA
CTCCCGAACCAACAGCTTCCTGAAGATTGTTCCAGAACCAAAGCAGCTTGAATGTAATCAACAGAAGG
TTGATGATGGTAAAAGGAGCTATCTTACTCAGTGGACAAAAGGAAATCAGAAACAAAGCCTGGAATGG
TTCACCCCAGTGGGGAAATTGTGGCTGACAGTGTCAGATTCCAGGTTGACAAGTGCTTTAAACACAAG
GTTAACATAAAGTTCTCTAACGAGCAGGGCTTACCTGGTTCCAATGCTAGTCTCTGTCTTCAAGCGGC
GCCTGTCTTATTCTGTGCCCTCAGGGCTGTGGATAGGAATGTCCTTCTACTGAAATCTGAACAACAGC
TGTCAGCTGAAAGTGTGTATAACATGGTTCCAAGTATAGAGCCGTATGGTTATTTCTACCATGGCCTC
AATCTTGATGATGGCAAGGAAGACCCTTGCATTCCTCAGAGGGATATGTTCTACAATGGTTTATATTA
CACACCTGTAAGCAACTATGGGGATGGAGATATCTATAATATTGTCAGGAACATGGGTCTAAAAGTCT
TTACCAATCTCCATTACCGAAAACCAGAAAAAATTATGGTCCAATGCGTAGTGTTCCGTCTAGAATTG
CATGTAGCTAGTGGAATCAGAGGGGAGAATGCTGACTATGTAGAACAGGCTATAATTCAAACAGTAAG
AACAAACTTCCCAGAGACATGGATGTGGGACCTCGTCAGTGTCGATTCCTCAGGCTCTGCCAATCTTT
CGTTCCTCATTCCTGATACGATAACCCAATGGGAGGCAAGTGGCTTTTGTGTGAATGGTGACGTTGGA
TTTGGCATTTCCTCTACAACCACTCTAGAAGTCTCCCAACCTTTCTTTATTGAGATTGCCTCACCCTT
TTCGGTTGTTCAAAATGAACAATTTGATTTGATTGTCAATGTCTTCAGCTACCGGAATACATGTGTAG
AGATTTCTGTTCAAGTGGAGGAGTCTCAGAATTATGAAGCAAATATTCATACCTTGAAAATCAATGGC
AGTGAGGTTATTCAAGCTGGAGGGAGGAAAACAAACGTCTGGACTATTATACCTAAGAAATTGGGTAA
AGTGAATATCACTGTAGTTGCTGAGTCCAAACAAAGCAGTGCTTGCCCAAATGAAGGAATGGAGCAGC
AAAAGCTAAACTGGAAAGACACTGTGGTCCAAAGCTTCTTAGTAGAGCCTGAAGGTATTGAAAAGGAA
AGGACCCAGAGTTTCCTTATCTGTACAGAAGGTGCCAAAGCCTCCAAGCAGGGAGTTTTGGACTTGCC
AAACGATGTAGTAGAAGGGTCAGCCAGAGGCTTTTTCACTGTTGTGGGGGATATTCTAGGACTTGCCT
TGCAGAATCTGGTTGTTCTCCAAATGCCCTATGGAAGTGGAGAGCAGAATGCTGCCCTACTAGCATCT
GATACTTATGTTCTGGACTATCTGAAATCTACTGAGCAACTGACAGAGGAAGTTCAATCTAAGGCTTT
CTTTCTCTTATCTAATGGTTATCAAAGGCAATTATCTTTCAAAAACTCTGATGGTTCCTATAGTGTGT
TTTGGCAGCAGAGTCAGAAAGGAAGCATATGGCTCAGTGCTCTTACTTTTAAGACATTGGAGAGAATG
AAAAAATATGTATTCATTGATGAAAATGTTCAAAAACAGACCTTAATCTGGCTTTCAAGCCAACAGAA
AACAAGCGGCTGCTTTAAGAATGATGGCCAGCTTTTCAACCACGCCTGGGAGGGTGGAGATGAAGAGG
ACATTTCACTCACTGCGTATGTTGTTGGGATGTTCTTTGAAGCTGGGCTCAATTTCACTTTTCCTGCT
CTACGAAACGCACTCTTTTGCCTTGAAGCGGCATTGGACAGTGGTGTCACTAATGGCTATAATCATGC
AATTCTAGCTTATGCTTTTGCCTTAGCTGGAAAAGAGAAGCAAGTGGAATCTTTACTCCAAACCCTGG
ATCAATCTGCCCCAAAACTAAATAATGTCATCTACTGGGAAAGAGAAAGGAAACCCAAGACAGAAGAA
TTTCCATCCTTTATTCCCTGGGCACCTTCTGCTCAGACTGAGAAGAGTTGCTACGTGCTGTTGGCTGT
CATTTCCCGGAAAATTCCTGACCTCACCTATGCTAGTAAGATTGTGCAGTGGCTTGCCCAACGGATGA
ATTCCCATGGAGGCTTTTCTTCCAACCAGACACCTGATGATACTCTGTTCAAATTATATACGGGCCAA
AAAGAAAGCTTTCGCTCTAGTTCTGTGGGCTATACACTGGGAAAAGCAAATGAAAAGAAGGAAAACAG
GAGAAATGGGGGTGAAGGATCCAGTGAGATTTTCCAGGTTAACGGTCATAACCGCCTACTGGTCCAAC
GTTCAGAAGTAACACAGGCACCTGGAGAATACACAGTAGATGTGGAAGGACACGGTTGTACATTTATC
CAGGCCACCCTTAAGTACAATGTTCTCCTACCTAAGAAGGCATCTGGATTTTCTCTTTCCTTGGAAAT
AGTAAAGAACTACTCTTCGACTGCTTTTGACCTCACAGTGACCCTCAAATACACTGGAATTCGCAATA
AATCCAGTATGGTGGTTATAGATGTAAAAATGCTATCAGGATTTACTCCAACCATGTCATCCATTGAA
GAGCTTGAAAACAAGGGCCAAGTGATGAAGACTGAAGTCAAGAATGACCATGTTCTTTTCTACTTGGA
AAATGTAGGTTTTGGTCGAGCAGACAGTTTCCCTTTTTCTGTTGAGCAGAGCAACCTTGTGTTCAACA
TTCAGCCAGCCCCAGCCATGGTCTACGATTATTATGAAAAAGAAGAATATGCCCTAGCTTTTTACAAC
ATCGACAGTAGTTCAGTTTCCGAGTGAGACAAAGCAATTACTAGAAGAGTTGGAGAAGCATTTCTTGT
AACAAACTGATTCTTCTGTATCAAACCTGGAAAAAAATCATGAACCATCTGACATCGTGAACAGTCTG
CAGTGGGCTATGGTTTCTTGTCAAGTCTTATTTCCTTATCATCCCATTAAATGTTGTCATTTTGCAAA
AAAAAAAAAAAAA

| | | | |
|---|---|---|---|
| NOV52b, CG59584-02 | SEQ ID NO:1094 | 1436 aa | MW at 161836.4 kD |
| Protein Sequence | | | |

SISIDINPELAPSVDMLVYSLHPGGEMVTDSTQFRIEKCFENQVNLNFSKEKSLPGSNIDLQVSAASN
SLCALWAVDQSVLLLRNYGQLSAQTVYSQLYSRELHGYYFRGLNLEDGLKVPCLEDEHILYNGIYYTP
AWADFGKDGYDLVKDPQNNRIFQRQNVTSFRNITQLSFQLISEPMFGDYWIVVKRNSRETVTHQFAVK
RYVLPKFEVTVNAPQTVTISDDEFQVDVCAKYNFGQPVQGETQIRVCREYFSSSNCEKNENEICEQFI
AQLENGCVSQIVNTKVFQLYRSGLFMTFHVAVIVTESGTVMQISEKTSVFITQLLGTVNFENMDTFYR
RGISYFGTLKFSDPNNVPMVNKLLQLELNDEFIGNYTTDENGEAQFSIDTSDIFDPEFNLKATYVRPE
SCYLPSWLTPQYLDAHFLVSRFYSRTNSFLKIVPEPKQLECNQQKVVTVHYSLNSEAYEDDSNVKFFY
LMMVKGAILLSGQKEIRNKAWNGNFSFPISISADLAPAAVLFVYTLHPSGEIVADSVRFQVDKCFKHK
VNIKFSNEQGLPGSNASLCLQAAPVLFCALRAVDRNVLLLKSEQQLSAESVYNMVPSIEPYGYFYHGL
NLDDGKEDPCIPQRDMFYNGLYYTPVSNYGDGDIYNIVRNMGLKVFTNLHYRKPEKIMVQCVVFRLEL
HVASGIRGENADYVEQAIIQTVRTNFPETWMWDLVSVDSSGSANLSFLIPDTITQWEASGFCVNGDVG
FGISSTTTLEVSQPFFIEIASPFSVVQNEQFDLIVNVFSYRNTCVEISVQVEESQNYEANIHTLKING
SEVIQAGGRKTNVWTIIPKKLGKVNITVVAESKQSSACPNEGMEQQKLNWKDTVVQSFLVEPEGIEKE
RTQSFLICTEGAKASKQGVLDLPNDVVEGSARGFFTVVGDILGLALQNLVVLQMPYGSGEQNAALLAS
DTYVLDYLKSTEQLTEEVQSKAFFLLSNGYQRQLSFKNSDGSYSVFWQQSQKGSIWLSALTFKTLERM
KKYVFIDENVQKQTLIWLSSQQKTSGCFKNDGQLFNHAWEGGDEEDISLTAYVVGMFFEAGLNFTFPA
LRNALFCLEAALDSGVTNGYNHAILAYAFALAGKEKQVESLLQTLDQSAPKLNNVIYWERERKPKTEE
FPSFIPWAPSAQTEKSCYVLLAVISRKIPDLTYASKIVQWLAQRMMSHGGFSSNQTPDDTLFKLYTGQ
KESFRSSSVGYTLGKANEKKENRRNGGEGSSEIFQVNGHNRLLVQRSEVTQAPGEYTVDVEGHGCTFI
QATLKYNVLLPKKASGFSLSLEIVKNYSSTAFDLTVTLKYTGIRNKSSMVVIDVKNLSGFTPTMSSIE
ELENKGQVMKTEVKNDHVLFYLENVGFGRADSFPFSVEQSNLVFNIQPAPAMVYDYYEKEEYALAFYN
IDSSSVSE

| | | |
|---|---|---|
| NOV52c 248210405 | SEQ ID NO:1095 | 571 bp |
| DNA Sequence | ORF Start: at 2 | ORF Stop: end of sequence |

CACCAGATGTGAAGGATCCAGTGAGATTTTCCACCTTAACGGTCATAATCGCCTACTGGTCCAACGTT
CAGAAGTAACACAGGCACCTGGAGAATACACAGTAGATGTGGAAGGACACGGTTGTACATTTATCCAG
GCCACCCTTAAGTACAATGTTCTCCTACCTAAGAAGGCATCTGGATTTTCTCTTTCCTTGGAAATAGT
AAAGAACTACTCTTCGACTGCTTTTGACCTCACAGTGACCCTCAAATACACTGGAATTCGCAATAAAT
CCAGTATGGTGGTTATAGATGTAAAAATGCTATCAGGATTTACTCCAACCATGTCATCCATTGAAGAG
CTTGAAAACAAGGGCCAAGTGATGAAGACTGAAGTCAAGAATGACCATGTTCTTTTCTACTTGGAAAA
TGGTTTTGGTCGAGCAGACAGTTTCCCTTTTTCTGTTGAGCAGAGCAACCTTGTGTTCAACATTCAGC
CAGCCCCAGCCATGGTCTACGATTACTATGAAAAAGAAGAATATGCCCTAGCTTTTTACAACATCGAC
AGTAGTTCAGTTTCCGAGCTCGAGGGC

| | | | |
|---|---|---|---|
| NOV52c, 248210405 | SEQ ID NO:1096 | 190 aa | MW at 21231.7 kD |
| Protein Sequence | | | |

TRSEGSSEIFQVNGHNRLLVQRSEVTQAPGEYTVDVEGHGCTFIQATLKYNVLLPKKASGFSLSLEIV
KNYSSTAFDLTVTLKYTGIRNKSSMVVIDVKMLSGFTPTMSSIEELENKGQVMKTEVKNDHVLFYLEN
GFGRASFPFSVEQSNLVFNIQPAPDYYEKEEYALAFYNIDSSSVSELEG

| | | |
|---|---|---|
| NOV52d, 248210436 | SEQ ID NO:1097 | 571 bp |
| DNA Sequence | ORF Start: at 2 | ORF Stop: end of sequence |

CACCAGATCTGAAGGATCCAGTGAGATTTTCCAGGTTAACGGTCATAACCGCCTACTGGTCCAACGTT
CAGAAGTAACACAGGCACCTGGACAATACACAGTAGATGTGGAAGGACACGGTTGTACATTTATCCAG
GCCACCCTTAAGTACAATGTTCTCCTACCTAAGAAGGCATCTGGATTTTCTCTTTCCTTGGAAATAGT
AAAGAACTACTCTTCGACTGCTTTTGACCTCACAGTGACCCTCAAATACACTGGAATTCGCAATAAAT
CCAGTATGGTGGTTATAGATGTAAAAATGCTATCAGGATTTACTCCAACCATGTCATCCATTGAAGAG
CTTGAAAACAAGGGCCAAGTGATGAAGACTGAAGTCAAGAATGACCATGTTCTTTTCTACTTGGAAAA
TGGTTTTGGTCGAGCAGACAGTTTCCCTTTTTCTGTTGAGCAGAGCAACCTTGTGTTCAACATTCAGC
CAGCCCCAGCCATGGTCTACGATTACTATGAAAAAGAAGAATATGCCCTAGCTTTTTACAACATCGAC
AGTAGTTCAGTTTCCGAGCTCGAGGGC

| | | | |
|---|---|---|---|
| NOV52d, 248210436 | SEQ ID NO:1098 | 190 aa | MW at 21230.7 kD |
| Protein Sequence | | | |

TRSEGSSEIFQVNGHNRLLVQRSEVTQAPGQYTVDVEGHGCTFIQATLKYNVLLPKKASGFSLSLEIV
KNYSSTAFDLTVTLKYTGIRNKSSMVVIDVKMLSGFTPTMSSIEELENKGQVMKTEVKNDHVLFYLEN
GFGRADSFPFSVEQSNLVFNIQPAPAMVYDYYEKEEYALAFYNIDSSSVSELEG

| | | |
|---|---|---|
| NOV52e 249357737 | SEQ ID NO:1099 | 571 bp |
| DNA Sequence | ORF Start: at 2 | ORF Stop: end of sequence |

CACCAGATCTGAAGGATCCAGTGAGATTTTCCAGGTTAACGGTCATAACCGCCTACTGGTCCAACGTT
CAGAAGTAACACAGGCACCTGGACAATACACAGTAGATGTGGAAGGACACGGTTGTACATTTATCCAG
GCCACCCTTAAGTACGATGTTCTCCTACCTAAGAAGGCATCTGGATTTTCTCTTTCCTTGGAAATAGT
AAAGAACTACTCTTCGACTGCTTTTGACCTCACAGTGACCCTCAAATACACTGGAATTCGCAATAAAT
CCAGTATGGTGGTTATAGATGTAAAAATGCTATCAGGATTTACTCCAACCATGTCATCCATTGAAGAG
CTTGAAAACAAGGGCCAAGTGATGAAGACTGAAGTCAAGAATGACCATGTTCTTTTCTACTTGGAAAA
TGGTTTTGGTCGAGCAGACAGTTTCCCTTTTTCTGTTGAGCAGAGCAACCTTGTGTTCAACATTCAGC
CAGCCCCAGCCATGGTCTACGATTACTATGAAAAAGAAGAATATGCCCTAGCTTTTTACAACATCGAC
AGTAGTTCAGTTTCCGAGCTCGAGGGC

| | | | |
|---|---|---|---|
| NOV52e, 249357737 | SEQ ID NO:1100 | 190 aa | MW at 21231.7 kD |
| Protein Sequence | | | |

TRSEGSSEIFQVNGHNRLLVQRSEVTQAPGQYTVDVEGHGCTFIQATLKYDVLLPKKASGFSLSLEIV
KNYSSTAFDLTVTLKYTGIRNKSSMVVIDVKMLSGFTPTMSSIEELENKGQVMKTEVKNDHVLFYLEN
GFGRADSFPFSVEQSNLVFNIQPAPDYYEKEEYALAFYNIDSSSVSELEG

| | | |
|---|---|---|
| NOV52f, 248210430 | SEQ ID NO:1101 | 529 bp |
| DNA Sequence | ORF Start: at 2 | ORF Stop: end of sequence |

CACCAGATCTGAAGGATCCAGTGAGATTTTCCAGGTTAACGGTCATAACCGCCTACTGGTCCAACGTT
CAGAAGTAACACAGGCACCTGGACAATACACAGTAGATGTGGAAGGACACGGTTGTACATTTATCCAG
GCCACCCTTAAGTACAATGTTCTCCTACCTAAGAAGGCATCTGGATTTTCTCTTTCCTTGGAAATAGT
AAAGAACTACTCTTCGACTGCTTTTGACCTCACAGTGACCCTCAAATACACTGGAATTCGCAATAAAT
CCAGTATGGTGGTTATAGATGTAAAAATGCTATCAGGATTTACTCCAACCATGTCATCCATTGAAGAG
CTTGAAAACAAGGGCCAAGTGATGAAGACTGAAGTCAAGAATGACCATGTTCTTTTCTACTTGGAAAA
TAGCAACCTTGTGTTCAACATTCAGCCAGCCCCAGCCATGGTCTACGATTACTATGAAAAAGAAGAAT
ATGCCCTAGCTTTTTACAACATCGACAGTAGTTCAGTTTCCGAGCTCGAGGGC

| | | | |
|---|---|---|---|
| NOV52f, 248210430 | SEQ ID NO:1102 | 176 aa | MW at 19705.1 kD |
| Protein Sequence | | | |

TRSEGSSEIFQVNGHNRLLVQRSEVTQAPGQYTVDVEGHGCTFIQATLKYNVLLPKKASGFSLSLEIV
KNYSSTAFDLTVTLKYTGIRNKSSMVVIDVKMLSGFTPTMSSIEELENKGQVMKTEVKNDHVLFYLEN
SNLVFNIQPAPAMVYDYYEKEEYALAFYNIDSSSVSELEG

| | | |
|---|---|---|
| NOV52g, 249357755 | SEQ ID NO:1103 | 547 bp |
| DNA Sequence | ORF Start: at 2 | ORF Stop: end of sequence |

CACCAGATCTGAAGGATCCAGTGAGATTTTCCAGGTTAACGGTCATAATCGCCTACTGGTCCAACGTT
CAGAAGTAACACAGGCACCTGGACAATACACAGTAGATGTGGAAGGACACGGTTGTACATTTATCCAG
GCCACCCTTAAGTACAATGTTCTCCTACCTAAGAAGGCATCTGGATTTTCTCTTTCCTTGGAAATAGT
AAAGAACTACTCTTCGACTGCTTTTGACCTCACAGTGACCCTCAAATACACTGGAATTCGCAATAAAT
CCAGTATGGTGGTTATAGATGTAAAAATGCTATCAGGATTTACTCCAACCATGTCATCCACTGAAGAG
CTTGAAAACAAGGGCCAAGTGATGAAGACTGAAGTCAAGAATGACCATGTTCTTTTCTACTTGGAAAA
TAGCAACCTTGTGTTCAACATTCAGCCAGCCCCAGCCATGGTCTACGATTACTATGAAAAAGAAGAAT
ATGCCCTAGCTTTTTACAACATCGACAGTAGTTCAGTTTCCGAGCTCGAGGGCAAGGGCGAATCCAGC
ACA

| | | | |
|---|---|---|---|
| NOV52g, 249357755 | SEQ ID NO:1104 | 182 aa | MW at 20282.6 kD |
| Protein Sequence | | | |

TRSEGSSEIFQVNGHNRLLVQRSEVTQAPGQYTVDVEGHGCTFIQATLKYNVLLPKKASGFSLSLEIV
KNYSSTAFDLTVTLKYTGIPNKSSMVVIDVKMLSGFTPTMSSTEELENKGQVMKTEVKNDHVLFYLEN
SNLVFNIQPAPAMVYDYYEKEEYALAFYNIDSSSVSELEGKGESST

| | | |
|---|---|---|
| NOV52h, CG59584-01 | SEQ ID NO:1105 | 4426 bp |
| DNA Sequence | ORF Start: ATG at 1 | ORF Stop: TGA at 4234 |

ATGTACTGTTTTTTGCGATCTGCCGTTTCGTTCTTCTGCCTCAGCCTCCCCAGGTGCTGGGGTTATAG
GTGTGAGCCACTGTGCCTGGCTATTCTTTTATTACAGTATGTTCTGCTGATTCCTTCTGTTCTACAAG
AAGGCTCTTTGGATAAAGCTTGTGCCCAGCTTTTTAATCTCACTGAATCTGTTGTTTTGACGGTCTCC
CTCAACTATGGTGAGGTCCAGACCAAAATATTTGAAGAAAATGTTACTGGAGAAAATTTCTTCAAATG
CATCAGCTTTGAGGTTCCTCAGGCCAGATCTGACCCACTGGCATTTATTACATTTTCTGCTAAAGGAG
CCACTCTCAACCTGGAAGAGAGGAGATCTGTGGCAATCAGATCCAGAGAGAATGTGGTCTTTGTACAG
ACTGATAAACCCACCTACAAGCCTGGACAGTATAATAAAAAGCCGATCAGTCACATAATGCCAGTGAT
AGCAGTCACTGAACAGGATCCAGAAGGCAATCGAATACAACAGTGGGTGAATGAGGAGTCTGTGGGAG
GGATTCTACAACTCTCCTTCCAGTTAATCTCAGAGCCCATCCTCGGATGGTATGAAATCACCGTGGAG
ATGCTCAATGAGAAGAAAACATATCACTCCTTCTCTGTGGAAGAATATGTGTTACCCAAATTTCAAAT
GACTGTGGATGCACCAGAAAATATCTTAGTTGTGGACTCTGAATTCAAAGTGAATGTCTGTGCCTTGT
ATACCTATGGTGAACCTGTGGACGGGAAGGTCCAACTTAGTGTGTGCAGAGAATCTACGGCTTATCAT
TCATGTGCTCATCTTATCAGTTCACTCTGTAAAAATTTTACCTTGGGGAAAGATGGCTGTGTCTCCAA
GTTTATTAACACAGATGCTTTTGAGTTAAATCGGGAAGGATACTGGAGTTTCCTCAAAGTGCATGCTC
TTGTTACAGAGCTTACAGGCTCCAAGTACGTATACATAGACTCATCAGTGGTGAAGATTAGTTTTGAG
ATGTAAATGGCATCGCTCAGTTTTTCTTGGACACATATACGTTTACATACCCAAATATCACTTTGAAA
GCAGCCTACAAGGCCAATGAAAATTGCCAGGCTCATGGCTGGGTGTTGCCTCAATACCCTCAGCCCGA
GTACTTTGCATATCGATTTTACTCCAAGATGAATAGCTTCCTAAAGATTGTCCAAGAGATGGAAGAAC
TGAGATGCAACCAGCAGAAGAGGGTGCTAGTGCACTGCATTCTCAATATGGAAGACTTTGAAGACAAA
ACCTACACAGCAGACTTCAATTATTTGGTGATTTCAAAAGGTGTAATCATTCTTCATGGGCAACAGAA
AATTGAGATCAACGAAAATGGGAGGAAGGGCATATTTTCCATTTCTATAGACATTAACCCTGAATTAG
CGCCCTCAGTACATATGCTTGTCTATAGCTTGCATCCTGGAGGAGAAATGGTCACTGATAGCACCCAA
TTCCAATTGAGAAATGTTAACATAAAGTTCTCTAACGAGCAGGGCTTACCTGGTTCCAATGCTAGTCT
CTGTCTTCAAGCGGCGCCTGTCTTATTCTGTGCCCTCAGGGCTGTGGATAGGAATGTCCTTCTACTGA
AATCTGAACAACAGCTGTCAGCTGAAAGTGTGTATAACATGGTTCCAAGTATAGAGCCGTATGGTTAT
TTCTACCATGGCCTCAATCTTGATGATGGCAAGGAAGACCCTTGCATTCCTCAGAGGGATATGTTCTA
CAATGGTTTATATTACACACCTGTAAGCAACTATGGGGATGGAGATATCTATAATATTGTCAGGAACA
TGGGTCTAAAAGTCTTTACCAATCTCCATTACCGAAAACCAGAAGTATGTGTGATGGAGAGAAGGCTG
CCACTCCCTAAGCCGCTTTATCTGGAAACAGAAAATTATGGTCCAATGCGTAGTGTTCCGTCTAGAAT
TGCATCTAGTGGAATCAGAGGGGAGAATGCTGACTATGTAGAACAGGCTATAATTCAAACAGTAAGAA
CAAACTTCCCAGAGACATGGATGTGGGACCTCGTCAGTGTCGATTCCTCAGGCTCTGCCAATCTTTCG
TTCCTCATTCCTGATACGATAACCCAATGGGAGGCAAGTGGCTTTTGTGTGAATGGTGACGTTGGATT
TGGCATTTCCTCTACAACCACTCTAGAAGTCTCCCAACCTTTCTTTATTGAGATTGCCTCACCCTTTT
CGGTTGTTCAAAATGAACAATTTGATTTGATTGTCAATGTCTTCAGCTACCGGAATACATGTGTAGAG
ATTTCTGTTCAAGTGGAGGAGTCTCAGAATTATGAAGCAAATATTCATACCTTGAAAATCAATGGCAG
TGAGGTTATTCAAGCTGGAGGGAGGAAAACAAACGTCTGGACTATTATACCTAAGAAATTGGGCAAAG
TGAATATCACTGTAGTTGCTGAGTCCAAACAAAGCAGTGCTTGCCCAAATGAAGGAATGGAGCAGCAA
AAGCTAAACTGGAAAGACACTGTGGTCCAAAGCTTCTTAGTAGAGCCTGAAGGTATTGAAAAGGAAAG
GACCCAGAGTTTCCTTATCTGTACAGAAGGTGCCAAAGCCTCCAAGCAGGGAGTTTTGGACTTGCCAA
ACGATGTAGTAGAAGGGTCAGCCAGAGGCTTTTTCACTGTTGTGGGGGATATTCTAGGACTTGCCTTG
CAGAATCTGGTTGTTCTCCAAATGCCCTATGGAAGTGGAGAGCAGAATGCTGCCCTACTAGCATCTGA
TACTTATGTTCTGGACTATCTGAAATCTACTGAGCAACTGACAGAGGAAGTTCAATCTAAGGCTTTCT
TTCTCTTATCTAATGGTTATCAAAGGCAATTATCTTTCAAAAACTCTGATGGTTCCTATAGTGTGTTT
TGGCAGCAGAGTCAGAAAGGAAGCATATGGCTCAGTGCTCTTACTTTTAAGACATTGGAGAGAATGAA
AAAATATGTATTCATTGATGAAAATGTTCAAAAACAGACCTTAATCTGGCTTTCAAGCCAACAGAAAA
CAAGCGGCTGCTTTAAGAATGATGGCCAGCTTTTCAACCACGCCTGGCAGGGTGGAGATGAAGAGGAC
ATTTCACTCACTGCGTATGTTGTTGGGATGTTCTTTGAAGCTGGGGCGGCATTGGACAGTGGTGTCAC
TAATGGCTATAATCATGCAATTCTAGCTTATGCTTTTGCCTTAGCTGGAAAAGAGAAGCAAGTGGAAT
CTTTACTCCAAACCCTGGATCAATCTGCCCCAAAACTAAATAATGTCATCTACTGGGAAAGAGAAAGG
AAACCCAAGACAGAAGAATTTCCATCCTTTATTCCCTGGGCACCTTCTGCTCAGACTGAGAAGAGTTG
CTACGTGCTGTTGGCTGTCATTTCCCGGAAAATTCCTGACCTCACCTATGCTAGTAAGATTGTGCAGT
GGCTTGCCCAACGGATGAATTCCCATGGAGGCTTTTCTTCCAACCAGGATCAAAACACTGTCACCTTT
AGCAGTGAAGGATCCAGTGAGATTTTCCAGGTTAACGGTCATAACCGCCTACTGGTCCAACGTTCAGA
AGTAACACAGGCACCTGGAGAATACACAGTAGATGTGGAAGGACACGGTTGTACATTTATCCAGGCCA
CCCTTAAGTACAATGTTCTCCTACCTAAGAAGGCATCTGGATTTTCTCTTTCCTTGGAAATAGTAAAG
AACTACTCTTCGACTGCTTTTGACCTCACAGTGACCCTCAAATACACTGGAATTCGCAATAAATCCAG
TATGGTGGTTATAGATGTAAAAATGCTATCAGGATTTACTCCAACCATGTCATCCATTGAAGAGCTTG
AAAACAAGGGCCAAGTGATGAAGACTGAAGTCAAGAATGACCATGTTCTTTTCTACTTGGAAAATGTA
GGTTTTGGTCGAGCAGACAGTTTCCCTTTTTCTGTTGAGCAGAGCAACCTTGTGTTCAACATTCAGCC
AGCCCCAGCCATGGTCTACGATTATTATGAAAAAGAAGAATATGCCCTAGCTTTTTACAACATCGACA
GTAGTTCAGTTTCCGAGTGAGACAAAGCAATTACTAGAAGAGTTGGAGAAGCATTTCTTGTAACAAAC
TGATTCTTCTGTATCAAACCTGGAAAAAAATCATGAACCATCTGACATCGTGAACAGTCTGCAGTGGG
CTATGGTTTCTTGTCAAGTCTTATTTCCTTATCATCCCATTAAATGTTGTCATTTTGCAAAAAAAAAA
AAAAAA

| | | | |
|---|---|---|---|
| NOV52h, CG59584-01 | SEQ ID NO:1106 | 1411 aa | MW at 158867.0 kD |
| Protein Sequence | | | |

MYCFLRSAVSFFCLSLPRCWGYRCEPLCLAILLLQYVLLIPSVLQEGSLDKACAQLFNLTESVVLTVS
LNYGEVQTKIFEENVTGENFFKCISFEVPQARSDPLAFITFSAKGATLNLEERRSVAIRSRENVVFVQ
TDKPTYKPGQYNKKPISHIMPVIAVTEQDPEGNRIQQWVNEESVGGILQLSFQLISEPTLGWYEITVE
MLNEKKTYHSFSVEEYVLPKFQMTVDAPENILVVDSEFKVNVCALYTYGEPVDGKVQLSVCRESTAYH
SCAHLISSLCKNFTLGKDGCVSKFINTDAFELNREGYWSFLKVHALVTELTGSKYVYIDSSVVKISFE
NMDMSYKQGLPYFGQIKLLNPDNSPIPNEVVQLHLKDKIVGNYTTDVNGIAQFFLDTYTFTYPNITLK
AAYKANENCQAHGWVLPQYPQPEYFAYRFYSKMNSFLKIVQENEELRCNQQKRVLVHCILNMEDFEDK
TYTADFNYLVISKGVIILHGQQKIEINENGRKGIFSISIDINPELAPSVHMLVYSLHPGGEMVTDSTQ
FQLRNVNIKFSNEQGLPGSNASLCLQAAPVLFCALRAVDRNVLLLKSEQQLSAESVYNMVPSIEPYGY
FYHGLNLDDGKEDPCIPQRDMFYNGLYYTPVSNYGDGDIYNIVRNMGLKVFTNLHYRKPEVCVMERRL
PLPKPLYLETENYGPMRSVPSRIASSGIRGENADYVEQAIIQTVRTNFPETWMWDLVSVDSSGSANLS
FLIPDTITQWEASGFCVNGDVGFGISSTTTLEVSQPFFIEIASPFSVVQNEQFDLIVNVFSYRNTCVE
ISVQVEESQNYEANIHTLKINGSEVIQAGGRKTNVWTIIPKKLGKVNITVVAESKQSSACPNEGMEQQ
KLNWKDTVVQSFLVEPEGIEKERTQSFLICTEGAKASKQGVLDLPNDVXTEGSARGFFTWGDILGLAL
QNLVVLQMPYGSGEQNAALLASDTYVLDYLKSTEQLTEEVQSKAFFLLSNGYQRQLSFKNSDGSYSVF
WQQSQKGSIWLSALTFKTLERMKKYVFIDENVQKQTLIWLSSQQKTSGCFKNDGQLFNHAWQGGDEED
ISLTAYVVGMFFEAGAALDSGVTNGYNHAILAYAFALAGKEKQVESLLQTLDQSAPKLNNVIYWERER
KPKTEEFPSFIPWAPSAQTEKSCYVLLAVISRKIPDLTYASKIVQWLAQRMNSHGGFSSNQDQNTVTF
SSEGSSEIFQVNGHNRLLVQRSEVTQAPGEYTVDVEGHGCTFIQATLKYNVLLPKKASGFSLSLEIVK
NYSSTAFDLTVTLKYTGIRNKSSMVVIDVKMLSGFTPTMSSIEELENKGQVMKTEVKNDHVLFYLENV
GFGRADSFPFSVEQSNLVFNIQPAPAMVYDYYEKEEYALAFYNIDSSSVSE

A ClustalW comparison of the above protein sequences yields the following sequence alignment shown in Table 52B.

TABLE 52B
Comparison of the NOV52 protein sequences.

| | | |
|---|---|---|
| NOV52a | MIKLLNPDNSPIPNEVVQLHLKDKIVGNYTTDVNGIAQFFLDTYTFTYPNITLKAAYKAN | (SEQ ID NO:1092) |
| NOV52b | ------------------------------------------------------------ | (SEQ ID NO:1094) |
| NOV52c | ------------------------------------------------------------ | (SEQ ID NO:1096) |
| NOV52d | ------------------------------------------------------------ | (SEQ ID NO:1098) |
| NOV52e | ------------------------------------------------------------ | (SEQ ID NO:1100) |
| NOV52f | ------------------------------------------------------------ | (SEQ ID NO:1102) |
| NOV52g | ------------------------------------------------------------ | (SEQ ID NO:1104) |
| NOV52h | ------------------------------------------------------------ | (SEQ ID NO:1106) |
| NOV52a | ENCQAHGWVLPQYPQPEYFAYRFYSKMNSFLKIVQEMEELRCNQQKRVLVHCILNMEDFE | |
| NOV52b | ------------------------------------------------------------ | |
| NOV52c | ------------------------------------------------------------ | |
| NOV52d | ------------------------------------------------------------ | |
| NOV52e | ------------------------------------------------------------ | |
| NOV52f | ------------------------------------------------------------ | |
| NOV52g | ------------------------------------------------------------ | |
| NOV52h | --------------------------MYCFLRSAVSFFCLSLPR---------------- | |
| NOV52a | DKTYTADFNYLVISKGVIILHGQQKIEINENGRKGIFSISIDINPELAPSVDMLVYSLHP | |
| NOV52b | -------------------------------------SISIDINPELAPSVDMLVYSLHP | |
| NOV52c | ------------------------------------------------------------ | |
| NOV52d | ------------------------------------------------------------ | |
| NOV52e | ------------------------------------------------------------ | |
| NOV52f | ------------------------------------------------------------ | |
| NOV52g | ------------------------------------------------------------ | |
| NOV52h | --------------------------------CWGYRCEPLCLAILLLQYVLLIPSVLQE | |
| NOV52a | GGEMVTDSTQFRIEKCFENQVNLNFSKEKSLPGSNIDLQVSAASNSLCALWAVDQSVLLL | |
| NOV52b | GGEMVTDSTQFRIEKCFENQVNLNFSKEKSLPGSNIDLQVSAASNSLCALWAVDQSVLLL | |
| NOV52c | ------------------------------------------------------------ | |
| NOV52d | ------------------------------------------------------------ | |
| NOV52e | ------------------------------------------------------------ | |
| NOV52f | ------------------------------------------------------------ | |
| NOV52g | ------------------------------------------------------------ | |
| NOV52h | GSLDKACAQLFNLTESVVLTVSLNYGEVQTK---IFEENVTGENFFKCISFEVPQARSDP | |
| NOV52a | RNYGQLSAQTVYSQLYSRELHGYYFRGLNLEDGLKVPCLEDEHILYNGIYYTPAWADFGK | |
| NOV52b | RNYGQLSAQTVYSQLYSRELHGYYFRGLNLEDGLKVPCLEDEHILYNGIYYTPAWADFGK | |
| NOV52c | ------------------------------------------------------------ | |
| NOV52d | ------------------------------------------------------------ | |
| NOV52e | ------------------------------------------------------------ | |
| NOV52f | ------------------------------------------------------------ | |
| NOV52g | ------------------------------------------------------------ | |
| NOV52h | LAFITFSAKGATLNLEERRSVAIRSRENVVFVQTDKPTYKPGQYNKKPISHIMPVIAVT- | |
| NOV52a | DGYDLVKDPQNNRIFQRQNVTSFRNITQLSFQLISEPMFGDYWIVVKRNSRETVTHQFAV | |
| NOV52b | DGYDLVKDPQNNRIFQRQNVTSFRNITQLSFQLTSEPMFGDYWIVVKRNSRETVTHQFAV | |
| NOV52c | ------------------------------------------------------------ | |
| NOV52d | ------------------------------------------------------------ | |
| NOV52e | ------------------------------------------------------------ | |
| NOV52f | ------------------------------------------------------------ | |
| NOV52g | ------------------------------------------------------------ | |
| NOV52h | -----EQDPEGNRIQQWVNEESVGGILQLSFQLISEPILGWYEITVEMLNEKKTYHSFSV | |
| NOV52a | KRYVLPKFEVTVNAPQTVTISDDEFQVDVCAKYNFGQPVQGETQIRVCREYFSSSNCEKN | |
| NOV52b | KRYVLPKFEVTVNAPQTVTISDDEFQVDVCAKYNFGQPVQGETQIRVCREYFSSSNCEKN | |
| NOV52c | ------------------------------------------------------------ | |
| NOV52d | ------------------------------------------------------------ | |
| NOV52e | ------------------------------------------------------------ | |
| NOV52f | ------------------------------------------------------------ | |
| NOV52g | ------------------------------------------------------------ | |
| NOV52h | EEYVLPKFQMTVDAPENILVVDSEFKVNVCALYTYGEPVDGKVQLSVCRESTAYHSCAHL | |
| NOV52a | ENEICEQFIAQLENGCVSQIVNTKVFQLYRSGLFMTFHVAVIVTESGTVMQISEKTSVFI | |
| NOV52b | ENEICEQFIAQLENGCVSQIVNTKVFQLYRSGLFMTFHVAVIVTESGTVMQISEKTSVFI | |
| NOV52c | ------------------------------------------------------------ | |
| NOV52d | ------------------------------------------------------------ | |
| NOV52e | ------------------------------------------------------------ | |
| NOV52f | ------------------------------------------------------------ | |
| NOV52g | ------------------------------------------------------------ | |
| NOV52h | ISSLCKNFTLGKD-GCVSKFINTDAFELNREGYWSFLKVEALVTE------LTGSKYVYI | |
| NOV52a | TQLLGTVNFENMDTFYRRGISYFGTLKFSDPNNVPMVNKLLQLELNDEFIGNYTTDENGE | |
| NOV52b | TQLLGTVNFENMDTFYRRGISYFGTLKFSDPNNVPMVNKLLQLELNDEFIGNYTTDENGE | |
| NOV52c | ------------------------------------------------------------ | |
| NOV52d | ------------------------------------------------------------ | |
| NOV52e | ------------------------------------------------------------ | |
| NOV52f | ------------------------------------------------------------ | |
| NOV52g | ------------------------------------------------------------ | |
| NOV52h | DSSVVKISFENMDMSYKQGLPYFGQIKLLNPDNSPIPNEVVQLHLKDKIVGNYTTDVNGI | |
| NOV52a | AQFSIDTSDIFDPEFNLKATYVRPESCYLPSWLTPQYLDAHFLVSRFYSRTNSFLKIVPE | |
| NOV52b | AQFSIDTSDIFDPEFNLKATYVRPESCYLPSWLTPQYLDAHFLVSRFYSRTNSFLKIVPE | |
| NOV52c | ------------------------------------------------------------ | |
| NOV52d | ------------------------------------------------------------ | |
| NOV52e | ------------------------------------------------------------ | |
| NOV52f | ------------------------------------------------------------ | |
| NOV52g | ------------------------------------------------------------ | |
| NOV52h | AQFFLDTYTFTYPNITLKAAYKANENCQAHGWVLPQYPQPEYFAYRFYSKMNSFLKIVQE | |
| NOV52a | PKQLECNQQKVVTVHYSLNSEAYEDDS-NVKFFYLMMVKGAILLSGQK--EIRNKAWNGN | |
| NOV52b | PKQLECNQQKVVTVHYSLNSEAYEDDS-NVKFFYLMMVKGAILLSGQK--EIRNKAWNGN | |
| NOV52c | ------------------------------------------------------------ | |
| NOV52d | ------------------------------------------------------------ | |
| NOV52e | ------------------------------------------------------------ | |
| NOV52f | ------------------------------------------------------------ | |
| NOV52g | ------------------------------------------------------------ | |
| NOV52h | MEELRCNQQKRVLVHCILNMEDFEDKTYTADFNYLVISKGVIILHGQQKIEINENGRKGI | |
| NOV52a | FSFPISISADLAPAAVLFVYTLHPSGEIVADSVRFQVDKCFKHKVNIKFSNEQGLPGSNA | |
| NOV52b | FSFPISISADLAPAAVLFVYTLHPSGEIVADSVRFQVDKCFKHKVNIKFSNEQGLPGSNA | |
| NOV52c | ------------------------------------------------------------ | |
| NOV52d | ------------------------------------------------------------ | |
| NOV52e | ------------------------------------------------------------ | |
| NOV52f | ------------------------------------------------------------ | |
| NOV52g | ------------------------------------------------------------ | |
| NOV52h | FSISIDINPELAPSVHMLVYSLHPGGEMVTDSTQFQLR-----NVNIKFSNEQGLPGSNA | |
| NOV52a | SLCLQAAPVLFCALRAVDRNVLLLKSEQQLSAESVYNMVPSIEPYGYFYHGLNLDDGKED | |
| NOV52b | SLCLQAAPVLFCALRAVDRNVLLLKSEQQLSAESVYNMVPSIEPYGYFYHGLNLDDGKED | |
| NOV52c | ------------------------------------------------------------ | |
| NOV52d | ------------------------------------------------------------ | |
| NOV52e | ------------------------------------------------------------ | |
| NOV52f | ------------------------------------------------------------ | |
| NOV52g | ------------------------------------------------------------ | |
| NOV52h | SLCLQAAPVLFCALRAVDRNVLLLKSEQQLSAESVYNMVPSIEPYGYFYHGLNLDDGKED | |
| NOV52a | PCIPQRDMFYNGLYYTPVSNYGDGDIYNIVPNMGLKVFTNLHYRKPEKIMVQCVVFR--- | |
| NOV52b | PCIPQRDMFYNGLYYTPVSNYGDGDIYNIVRNMGLKVFTNLHYRKPEKIMVQCVVFR--- | |
| NOV52c | ------------------------------------------------------------ | |
| NOV52d | ------------------------------------------------------------ | |
| NOV52e | ------------------------------------------------------------ | |
| NOV52f | ------------------------------------------------------------ | |
| NOV52g | ------------------------------------------------------------ | |
| NOV52h | PCIPQRDMFYNGLYYTPVSNYGDGDIYNIVRNMGLKVFTNLHYRKPEVCVMERRLPLPKP | |
| NOV52a | --------------LELHVASGIRGENADYVEQAIIQTVRTNFPETWMWDLVSVDSSGSA | |
| NOV52b | --------------LELRVASGIRGENADYVEQAIIQTVRTNFPETWMWDLVSVDSSGSA | |
| NOV52c | ------------------------------------------------------------ | |
| NOV52d | ------------------------------------------------------------ | |
| NOV52e | ------------------------------------------------------------ | |
| NOV52f | ------------------------------------------------------------ | |
| NOV52g | ------------------------------------------------------------ | |
| NOV52h | LYLETENYGPMRSVPSRIASSGIRGENADYVEQAIIQTVRTNFPETWMWDLVSVDSSGSA | |
| NOV52a | NLSFLIPDTITQWEASGFCVNGDVGFGISSTTTLEVSQPFFIEIASPFSVVQNEQFDLIV | |
| NOV52b | NLSFLIPDTITQWEASGFCVNGDVGFGISSTTTLEVSQPFFIEIASPFSVVQNEQFDLIV | |
| NOV52c | ------------------------------------------------------------ | |
| NOV52d | ------------------------------------------------------------ | |
| NOV52e | ------------------------------------------------------------ | |
| NOV52f | ------------------------------------------------------------ | |
| NOV52g | ------------------------------------------------------------ | |
| NOV52h | NLSFLIPDTITQWEASGFCVNGDVGFGISSTTTLEVSQPFFIEIASPFSVVQNEQFDLIV | |
| NOV52a | NVFSYRNTCVEISVQVEESQNYEANIHTLKINGSEVIQAGGRKTNVWTIIPKKLGKVNIT | |
| NOV52b | NVFSYRNTCVEISVQVEESQNYEANIHTLKINGSEVIQAGGRKTNVWTIIPKKLGKVNIT | |
| NOV52c | ------------------------------------------------------------ | |
| NOV52d | ------------------------------------------------------------ | |
| NOV52e | ------------------------------------------------------------ | |
| NOV52f | ------------------------------------------------------------ | |
| NOV52g | ------------------------------------------------------------ | |
| NOV52h | NVFSYRNTCVEISVQVEESQNYEANIHTLKINGSEVIQAGGRKTNVWTIIPKKLGKVNIT | |
| NOV52a | VVAESKQSSACPNEGMEQQKLNWKDTVVQSFLVEPEGIEKERTQSFLICTEGAKASKQGV | |
| NOV52b | VVAESKQSSACPNEGMEQQKLNWKDTVVQSFLVEPEGIEKERTQSFLICTEGAKASKQGV | |
| NOV52c | ------------------------------------------------------------ | |
| NOV52d | ------------------------------------------------------------ | |
| NOV52e | ------------------------------------------------------------ | |
| NOV52f | ------------------------------------------------------------ | |
| NOV52g | ------------------------------------------------------------ | |
| NOV52h | VVAESKQSSACPNEGMEQQKLNWKDTVVQSFLVEPEGIEKERTQSFLICTEGAKASKQGV | |
| NOV52a | LDLPNDVVEGSARGFFTVVGDILGLALQNLVVLQMPYGSGEQNAALLASDTYVLDYLKST | |
| NOV52b | LDLPNDVVEGSARGFFTVVGDILGLALQNLVVLQMPYGSGEQNAALLASDTYVLDYLKST | |
| NOV52c | ------------------------------------------------------------ | |
| NOV52d | ------------------------------------------------------------ | |
| NOV52e | ------------------------------------------------------------ | |
| NOV52f | ------------------------------------------------------------ | |
| NOV52g | ------------------------------------------------------------ | |
| NOV52h | LDLPNDVVEGSARGFFTVVGDILGLALQNLVVLQMPYGSGEQNAALLASDTYVLDYLKST | |
| NOV52a | EQLTEEVQSKAFFLLSNGYQRQLSFKNSDGSYSVFWQQSQKGSIWLSALTFKTLERMKKY | |
| NOV52b | EQLTEEVQSKAFFLLSNGYQRQLSFKNSDGSYSVFWQQSQKGSIWLSALTFKTLERMKKY | |
| NOV52c | ------------------------------------------------------------ | |
| NOV52d | ------------------------------------------------------------ | |
| NOV52e | ------------------------------------------------------------ | |
| NOV52f | ------------------------------------------------------------ | |
| NOV52g | ------------------------------------------------------------ | |
| NOV52h | EQLTEEVQSKAFFLLSNGYQRQLSFKNSDGSYSVFWQQSQKGSIWLSALTFKTLERMKKY | |
| NOV52a | VFIDENVQKQTLIWLSSQQKTSGCFKNDGQLFNHAWEGGDEEDISLTAYVVGMFFEAGLN | |
| NOV52b | VFIDENVQKQTLIWLSSQQKTSGCFKNDGQLFNHAWEGGDEEDISLTAYVVGMFFEAGLN | |
| NOV52c | ------------------------------------------------------------ | |
| NOV52d | ------------------------------------------------------------ | |
| NOV52e | ------------------------------------------------------------ | |
| NOV52f | ------------------------------------------------------------ | |
| NOV52g | ------------------------------------------------------------ | |
| NOV52h | VFIDENVQKQTLIWLSSQQKTSGCFKNDGQLFNHAWQGGDEEDISLTAYVVGMFFEAGA- | |
| NOV52a | FTFPALRNALFCLEAALDSGVTNGYNHAILAYAFALAGKEKQVESLLQTLDQSAPKLNNV | |
| NOV52b | FTFPALRNALFCLEAALDSGVTNGYNHAILAYAFALAGKEKQVESLLQTLDQSAPKLNNV | |
| NOV52c | ------------------------------------------------------------ | |
| NOV52d | ------------------------------------------------------------ | |
| NOV52e | ------------------------------------------------------------ | |
| NOV52f | ------------------------------------------------------------ | |
| NOV52g | ------------------------------------------------------------ | |
| NOV52h | ---------------ALDSGVTNGYNHAILAYAFALAGKEKQVESLLQTLDQSAPKLNNV | |
| NOV52a | IYWERERKPKTEEFPSFIPWAPSAQTEKSCYVLLAVISRKIPDLTYASKIVQWLAQRMNS | |
| NOV52b | IYWERERKPKTEEFPSFIPWAPSAQTEKSCYVLLAVISRKIPDLTYASKIVQWLAQRMNS | |
| NOV52c | ------------------------------------------------------------ | |
| NOV52d | ------------------------------------------------------------ | |
| NOV52e | ------------------------------------------------------------ | |
| NOV52f | ------------------------------------------------------------ | |
| NOV52g | ------------------------------------------------------------ | |
| NOV52h | IYWERERKPKTEEFPSFIPWAPSAQTEKSCYVLLAVISRKIPDLTYASKIVQWLAQRMNS | |
| NOV52a | HGGFSSNQTPDDTLFKLYTGQKESFRSSSVGYTLGKANEKKENRRNGGEGSSEIFQVNGH | |
| NOV52b | HGGFSSNQTPDDTLFKLYTGQKESFRSSSVGYTLGKANEKKENRRNGGEGSSEIFQVNGH | |
| NOV52c | ------------------------TRS---------------------EGSSEIFQVNGH | |
| NOV52d | ------------------------TRS---------------------EGSSEIFQVNGH | |
| NOV52e | ------------------------TRS---------------------EGSSEIFQVNGH | |
| NOV52f | ------------------------TRS---------------------EGSSEIFQVNGH | |
| NOV52g | ------------------------TRS---------------------EGSSEIFQVNGH | |
| NOV52h | HGGFSSNQDQNT----------VTFSS---------------------EGSSEIFQVNGH | |
| NOV52a | NRLLVQRSEVTQAPGEYTVDVEGHGCTFIQATLKYNVLLPKKASGFSLSLEIVKNYSSTA | |
| NOV52b | NRLLVQRSEVTQAPGEYTVDVEGHGCTFIQATLKYNVLLPKKASGFSLSLEIVKNYSSTA | |
| NOV52c | NRLLVQRSEVTQAPGEYTVDVEGHGCTFIQATLKYNVLLPKKASGFSLSLEIVKNYSSTA | |
| NOV52d | NRLLVQRSEVTQAPGQYTVDVEGHGCTFIQATLKYNVLLPKKASGFSLSLEIVKNYSSTA | |
| NOV52e | NRLLVQRSEVTQAPGQYTVDVEGHGCTFIQATLKYDVLLPKKASGFSLSLEIVKNYSSTA | |
| NOV52f | NRLLVQRSEVTQAPGQYTVDVEGHGCTFIQATLKYNVLLPKKASGFSLSLEIVKNYSSTA | |
| NOV52g | NRLLVQRSEVTQAPGQYTVDVEGHGCTFIQATLKYNVLLPKKASGFSLSLEIVKNYSSTA | |
| NOV52h | NRLLVQRSEVTQAPGEYTVDVEGHGCTFIQATLKYNVLLPKKASGFSLSLEIVKNYSSTA | |
| NOV52a | FDLTVTLKYTGIRNKSSMVVIDVKMLSGFTPTMSSIEELENKGQVMKTEVKNDHVLFYLE | |
| NOV52b | FDLTVTLKYTGIRNKSSMVVIDVKMLSGFTPTMSSIEELENKGQVMKTEVKNDHVLFYLE | |
| NOV52C | FDLTVTLKYTGIRNKSSMVVIDVKMLSGFTPTMSSIEELENKGQVMKTEVKNDHVLFYLE | |
| NOV52d | FDLTVTLKYTGIRNKSSMVVIDVKMLSGFTPTMSSIEELENKGQVMKTEVKNDHVLFYLE | |
| NOV52e | FDLTVTLKYTGIRNKSSMVVIDVKMLSGFTPTMSSIEELENKGQVMKTEVKNDHVLFYLE | |
| NOV52f | FDLTVTLKYTGIRNKSSMVVIDVKMLSGFTPTMSSIEELENKGQVMKTEVKNDHVLFYLE | |
| NOV52g | FDLTVTLKYTGIRNKSSMVVIDVKMLSGFTPTMSSTEELENKGQVMKTEVKNDHVLFYLE | |
| NOV52h | FDLTVTLKYTGIRNKSSMVVIDVKMLSGFTPTMSSIEELENKGQVMKTEVKNDHVLFYLE | |
| NOV52a | NVGFGRADSFPFSVEQSNLVFNIQPAPAMVYDYYEKEEYALAFYNIDSSSVSE------- | |
| NOV52b | NVGFGRADSFPFSVEQSNLVFNIQPAPAMVYDYYEKEEYALAFYNIDSSSVSE------- | |
| NOV52c | NG-FGRADSFPFSVEQSNLVFNIQPAPAMVYDYYEKEEYALAFYNIDSSSVSELEG---- | |
| NOV52d | NG-FGRADSFPFSVEQSNLVFNIQPAPAMVYDYYEKEEYALAFYNIDSSSVSELEG---- | |
| NOV52e | NG-FGRADSFPFSVEQSNLVFNIQPAPAMVYDYYEKEEYALAFYNIDSSSVSELEG---- | |
| NOV52f | NS---------------NLVFNIQPAPAMVYDYYEKEEYALAFYNIDSSSVSELEG---- | |
| NOV52g | NS---------------NLVFNIQPAPAMVYDYYEKEEYALAFYNIDSSSVSELEGKGES | |
| NOV52h | NVGFGRADSFPFSVEQSNLVFNIQPAPAMVYDYYEKEEYALAFYNIDSSSVSE------- | |
| NOV52a | -- | |
| NOV52b | -- | |
| NOV52c | -- | |
| NOV52d | -- | |
| NOV52e | -- | |
| NOV52f | -- | |
| NOV52g | ST | |
| NOV52h | -- | |

Further analysis of the NOV52a protein yielded the following properties shown in Table 52C.

TABLE 52C
Protein Sequence Properties NOV52a

| | |
|---|---|
| SignalP analysis: | No Known Signal Sequence Predicted |
| PSORT II analysis: | |

| PSG: | a new signal peptide prediction method | |
|---|---|---|
| | N-region: | length 8; pos. chg 1; neg. chg 1 |
| | H-region: | length 6; peak value −6.21 |
| | PSG score: | −10.61 |

| | |
|---|---|
| GvH: | von Heijne's method for signal seq. recognition |
| | GvH score (threshold: −2.1): −9.40 |
| | possible cleavage site: between 45 and 46 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 3 | |
| | Number of TMS(s) for threshold 0.5: 0 | |
| | PERIPHERAL | Likelihood = 1.11 (at 629) |

| | ALOM score: −1.70 (number of TMSs: 0) | | | |
|---|---|---|---|---|
| MITDISC: | discrimination of mitochondrial targeting seq | | | |
| | R content: | 0 | Hyd Moment(75): | 9.53 |
| | Hyd Moment(95): | 7.08 | G content: | 0 |
| | D/E content: | 2 | S/T content: | 1 |

| | |
|---|---|
| | Score: −6.16 |
| Gavel: | prediction of cleavage sites for mitochondrial preseq |
| | cleavage site motif not found |

NUCDISC: discrimination of nuclear localization signals

| | |
|---|---|
| | pat4: RKPK (4) at 1307 |
| | pat7: none |
| | bipartite: none |
| | content of basic residues: 8.7% |
| | NLS Score: −0.22 |
| KDEL: | ER retention motif in the C-terminus: none |

ER Membrane Retention Signals: none
SKL: peroxisomal targeting signal in the C-terminus: none
PTS2: 2nd peroxisomal targeting signal: found
KIVPEPKQL at 596
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 89 |
| COIL: | Lupas's algorithm to detect coiled-coil regions |
| | total: 0 residues |

Final Results (k = 9/23):

47.8%: nuclear
30.4%: cytoplasmic
17.4%: mitochondrial
4.3%: vesicles of secretory system

>> prediction for CG59584-03 is nuc (k = 23)

A search of the NOV52a protein against the Geneseq database, a proprietary database that contains sequences published in patents and patent publication, yielded several homologous proteins shown in Table 52D.

TABLE 52D
Geneseq Results for NOV52a

| | | NOV52a | Identities/ | | |
|---|---|---|---|---|---|
| | | Residues/ | Similarities for | | |
| Geneseq | Protein/Organism/Length | Match | the Matched | | Expect |
| Identifier | [Patent #, Date] | Residues | Region | | Value |
| ABU52373 | Human GPCR related protein | 158 . . . 1593 | 1436/1436 | (100%) | 0.0 |
| | NOV26b - Homo sapiens, 1436 aa. | 1 . . . 1436 | 1436/1436 | (100%) | |
| | [WO200279398-A2, 10-OCT-2002] | | | | |
| ABU52372 | Human GPCR related protein | 307 . . . 1593 | 997/1311 | (76%) | 0.0 |
| | NOV26a - Homo sapiens, 1411 aa. | 164 . . . 1411 | 1072/1311 | (81%) | |
| | [WO200279398-A2, 10-OCT-2002] | | | | |
| ABG05715 | Novel human diagnostic protein | 308 . . . 1137 | 742/852 | (87%) | 0.0 |
| | #5706 - Homo sapiens, 1200 aa. | 30 . . . 819 | 748/852 | (87%) | |
| | [WO200175067-A2, 11-OCT-2001] | | | | |
| ABG05716 | Novel human diagnostic protein | 393 . . . 1137 | 655/767 | (85%) | 0.0 |
| | #5707 - Homo sapiens, 1379 aa. | 294 . . . 998 | 662/767 | (85%) | |
| | [WO200175067-A2, 11-OCT-2001] | | | | |
| AAU81018 | Human alpha2 macroglobulin | 306 . . . 1589 | 527/1320 | (39%) | 0.0 |
| | (alpha2M) receptor #1 mature protein - | 144 . . . 1445 | 798/1320 | (59%) | |
| | Homo sapiens, 1451 aa. | | | | |
| | [WO200192474-A1, 06-DEC-2001] | | | | |

In a BLAST search of public sequence databases, the NOV52a protein was found to have homology to the proteins shown in the BLASTP data in Table 52E.

TABLE 52E
Public BLASTP Results for NOV52a

| | | NOV52a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P20740 | Ovostatin precursor | 306 . . . 1584 | 577/1306 (44%) | 0.0 |
| | (Ovomacroglobulin) - Gallus gallus | 178 . . . 1461 | 844/1306 (64%) | |
| | (Chicken), 1473 aa. | | | |
| P01023 | Alpha-2-macroglobulin precursor | 306 . . . 1589 | 527/1320 (39%) | 0.0 |
| | (Alpha-2-M) - Homo sapiens | 167 . . . 1468 | 798/1320 (59%) | |
| | (Human), 1474 aa. | | | |
| CAA01532 | ALPHA 2-MACROGLOBULIN 690- | 306 . . . 1589 | 525/1320 (39%) | 0.0 |
| | 730 - Homo sapiens (Human), 1474 | 167 . . . 1468 | 795/1320 (59%) | |
| | aa. | | | |
| CAA01533 | ALPHA 2-MACROGLOBULIN 690- | 306 . . . 1589 | 524/1330 (39%) | 0.0 |
| | 740 - Homo sapiens (Human), 1484 | 167 . . . 1478 | 792/1330 (59%) | |
| | aa. | | | |
| P06238 | Alpha-2-macroglobulin precursor | 306 . . . 1590 | 520/1314 (39%) | 0.0 |
| | (Alpha-2-M) - Rattus norvegicus | 170 . . . 1467 | 782/1314 (58%) | |
| | (Rat), 1472 aa. | | | |

PFam analysis predicts that the NOV52a protein contains the domains shown in the Table 52F.

TABLE 52F
Domain Analysis of NOV52a

| | | Identities/ | |
|---|---|---|---|
| | | Similarities | |
| Pfam | NOV52a Match | for the | |
| Domain | Region | Matched Region | Expect Value |
| A2M_N | 110 . . . 763 | 171/707 (24%) | 6e−62 |
| | | 420/707 (59%) | |
| A2M | 852 . . . 1584 | 258/858 (30%) | 3.9e−146 |
| | | 503/858 (59%) | |

### Example 53

The NOV53 clone was analyzed, and the nucleotide and encoded polypeptide sequences are shown in Table 53A.

TABLE 53A
NOV53 Sequence Analysis

| | | |
|---|---|---|
| NOV53a, 56008-01 | SEQ ID NO:1107 | 3445 bp |
| DNA Sequence | ORF Start: ATG at 117 | ORF Stop: TGA at 2381 |

CACCGCGTGTTCGCGCCTGGTAGAGATTTCTCGAAGACACCAGTGGGCCCGTGTGGAACC
AAACCTGCGCGCGTGGCCGGGCCGTGGGACAACGAGGCCGCGGAGACGAAGGCGCAATGG
CGAGGAAGTTATCTGTAATCTTGATCCTGACCTTTGCCCTCTCTGTCACAAATCCCCTTC
ATGAACTAAAAGCAGCTGCTTTCCCCCAGACCACTGAGAAAATTAGTCCGAATTGGGAAT
CTGGCATTAATGTTGACTTGGCAATTTCCACACGGCAATATCATCTACAACAGCTTTTCT
ACCGCTATGGAGAAAATAATTCTTTGTCAGTTGAAGGGTTCAGAAAATTACTTCAAAATA
TAGGCATAGATAAGATTAAAAGAATCCATATACACCATGACCACGACCATCACTCAGACC
ACGAGCATCACTCAGACCATGAGCGTCACTCAGACCATGAGCATCACTCAGAGCACGAGC
ATCACTCTGACCATGATCATCACTCTCACCATAATCATGCTGCTTCTGGTAAAAATAAGC
GAAAAGCTCTTTGCCCAGACCATGACTCAGATAGTTCAGGTAAAGATCCTAGAAACAGCC
AGGGGAAAGGAGCTCACCGACCAGAACATGCCAGTGGTAGAAGGAATGTCAAGGACAGTG
TTAGTGCTAGTGAAGTGACCTCAACTGTGTACAACACTGTCTCTGAAGGAACTCACTTTC
TAGAGACAATAGAGACTCCAAGACCTGGAAAACTCTTCCCCAAAGATGTAAGCAGCTCCA
CTCCACCCAGTGTCACATCAAAGAGCCGGGTGAGCCGGCTGGCTGGTAGGAAAACAAATG
AATCTGTGAGTGAGCCCCGAAAAGGCTTTATGTATTCCAGAAACACAAATGAAAATCCTC
AGGAGTGTTTCAATGCATCAAAGCTACTGACATCTCATGGCATGGGCATCCAGGTTCCGC
TGAATGCAACAGAGTTCAACTATCTCTGTCCAGCCATCATCAACCAAATTGATGCTAGAT
CTTGTCTGATTCATACAAGTGAAAAGAAGGCTGAAATCCCTCCAAAGACCTATTCATTAC
TGGCACTGGCCGTTGGGACTTTGAGTGGTGATGCTTTTTTACACCTTCTTCCACATTCTC
ATGCAAGTCACCACCATAGTCATAGCCATGAAGAACCAGCAATGGAAATGAAAAGAGGAC
CACTTTTCAGTCATCTGTCTTCTCAAAACATAGAAGAAAGTGCCTATTTTGATTCCACGT
GGAAGGGTCTAACAGCTCTAGGAGGCCTGTATTTCATGTTTCTTGTTGAACATGTCCTCA
CATTGATCAAACAATTTAAAGATAAGAAGAAAAAGAATCAGAAGAAACCTGAAAATGATG
ATGATGTGGAGATTAAGA2GCAGTTGTCCAAGTATGAATCTCAACTTTCAACAAATGAGG
AGAAAGTAGATACAGATGATCGAACTGAAGGCTATTTACGAGCAGACTCACAAGAGCCCT
CCCACTTTGATTCTCAGCAGCCTGCAGTCTTGGAAGAAGAAGAGGTCATGATAGCTCATG
CTCATCCACAGGAAGTCTACAATGAATATGTACCCAGAGGGTGCAAGAATAAATGCCATT
CACATTTCCACGATACACTCGGCCAGTCAGACGATCTCATTCACCACCATCATGACTACC
ATCATATTCTCCATCATCACCACCACCAAAACCACCATCCTCACAGTCACAGCCAGCGCT
ACTCTCGGGAGGAGCTGAAAGATGCCGGCGTCGCCACTCTGGCCTGGATGGTGATAATGG
GTGATGGCCTGCACAATTTCAGCGATGGCCTAGCAATTGGTGCTGCTTTTACTGAAGGCT
TATCAAGTGGTTTAAGTACTTCTGTTGCTGTGTTCTGTCATGAGTTGCCTCATGAATTAG
GTGACTTTGCTGTTCTACTAAAGGCTGGCATGACCGTTAAGCAGGCTGTCCTTTATAATG
CATTGTCAGCCATGCTGGCGTATCTTGGAATGGCAACAGGAATTTTCATTGGTCATTATG
CTGAAAATGTTTCTATGTGGATATTTGCACTTACTGCTGGCTTATTCATGTATGTTGCTC
TGGTTGATATGGTACCTGAAATGCTGCACAATGATGCTAGTGACCATGGATGTAGCCGCT
GGGGGTATTTCTTTTTACAGAATGCTGGGATGCTTTTGGGTTTTGGAATTATGTTACTTA
TTTCCATATTTGAACATAAAATCGTGTTTCGTATAAATTTCTAGTTAAGGTTTAAATGCT
AGAGTAGCTTAAAAAGTTGTCATAGTTTCAGTAGGTCATAGGGAGATGAGTTTGTATGCT
GTACTATGCAGCGTTTAAAGTTAGTGGGTTTTGTGATTTTTGTATTGAATATTGCTGTCT
GTTACAAAGTCAGTTAAAGGTACGTTTTAATATTTAAGTTATTCTATCTTGGAGATAAAA
TCTGTATGTGCAATTCACCGGTATTACCAGTTTATTATGTAAACAAGAGATTTGGCATGA
CATGTTCTGTATGTTTCAGGGAAAAATGTCTTTAATGCTTTTTCAAGAACTAACACAGTT
ATTCCTATACTGGATTTTAGGTCTCTGAAGAACTGCTGGTGTTTAGGAATAAGAATGTGC
ATGAAGCCTAAAATACCAAGAAAGCTTATACTGAATTTAAGCAAAGAAATAAAGGAGAAA
AGAGAAGAATCTGAGAATTGGGGAGGCATAGATTCTTATAAAAATCACAAAATTTGTTGT
AAATTAGAGGGGAGAAATTTAGAATTAAGTATAAAAAGGCAGAATTAGTATAGAGTACAT
TCATTAAACATTTTTGTCAGGATTATTTCCCGTAAAAACGTAGTGAGCACTTTTCATATA
CTAATTTAGTTGTACATTTAACTTTGTATAATACAGAAATCTAAATATATTTAATGAATT
CAAGCAATATATCACTTGACCAAGAAATTGGAATTTCAAAATGTTCGTGCGGGTATATAC
CAGATGAGTACAGTGAGTAGTTTTATGTATCACCAGACTGGGTTATTGCCAAGTTATATA
TCACCAAAAGCTGTATGACTGGATGTTCTGGTTACCTGGTTTACAAAATTATCAGAGTAG
TAAAACTTTGATATATATGAGGATATTAAAACTACACTAAGTATCATTTGATTCGATTCA
GAAAGTACTTTGATATCTCTCAGTGCTTCAGTGCTATCATTGTGAGCAATTGTCTTTTAT
ATACGGTACTGTAGCCATACTAGGCCTGTCTGTGGCATTCTCTAGATGTTTCTTTTTTAC
ACAATAAATTCCTTATATCAGCTTG

| | | |
|---|---|---|
| NOV53a, CG56008-01 | SEQ ID NO:1108 | 775 aa |
| Protein Sequence | | |

MARKLSVILILTFALSVTNPLHELKAAAFPQTTEKISPNWESGINVDLAISTRQYHLQQL
FYRYGENNSLSVEGFRKLLQNIGIDKIKRIHIHHDHDHHSDHEHHSDHERHSDHEHHSEH
EHHSDHDHHSHHNHAASGKNKRKALCPDHDSDSSGKDPRNSQGKGAHRPEHASGRRNVKD
SVSASEVTSTVYNTVSEGTHFLETIETPRPGKLFPKDVSSSTPPSVTSKSRVSRLAGRKT
NESVSEPRKGFMYSRNTNENPQECFNASKLLTSHGMGIQVPLNATEFNYLCPAIINQIDA
RSCLIHTSEKKAEIPPKTYSLQTAWVGGFIAISIISFLSLLGVILVPLMNRVFFKFLLSF
LVALAVGTLSGDAFLHLLPHSHASHHHSHSHEEPAMEMKRGPLFSHLSSQNIEESAYFDS
TWKGLTALGGLYFMFLVEHVLTLIKQFKDKKKKNQKKPENDDDVEIKKQLSKYESQLSTN
EEKVDTDDRTEGYLRADSQEPSHFDSQQPAVLEEEEVMIAHAHPQEVYNEYVPRGCKNKC
HSHFHDTLGQSDDLIHHHHDYHHILHHHHHQNHHPHSHSQRYSREELKDAGVATLAWMVI
MGDGLHNFSDGLAIGAAFTEGLSSGLSTSVAVFCHELPHELGDFAVLLKAGMTVKQAVLY
NALSAMLAYLGMATGIFIGHYAENVSMWIFALTAGLFMYVALVDMVPENLHNDASDHGCS
RWGYFFLQNAGMLLGFGIMLLISIFEHKIVFRINF

Further analysis of the NOV53a protein yielded the following properties shown in Table 53B.

TABLE 53B
Protein Sequence Properties NOV53a

| | |
|---|---|
| SignalP analysis: | Cleavage site between residues 18 and 19 |
| PSORT II analysis: | |

Results Summary:
plasma membrane --- Certainty = 0.6400(Affirmative) < succ>
Golgi body --- Certainty = 0.4600(Affirmative) < succ>
endoplasmic reticulum (membrane) --- Certainty =
0.3700(Affirmative) < succ>
endoplasmic reticulum (lumen) --- Certainty =
0.1000(Affirmative) < succ>
Reasoning Steps:
>>> MUS belongs to the animal class
*** Reasoning Step: 2

| | |
|---|---|
| SRCFLG: | 1 |

Prelim. Calc. of ALOM (thresh: 0.5) count: 7

| | |
|---|---|
| mostN: | 5 at i = 4 |

| | |
|---|---|
| MTOP program | Imiddle: 12 Chg diff(C-N): −2.5 |

| | |
|---|---|
| McG: | Length of UR: 17 |
| | Peak Value of UR: 3.09 |
| | Net Charge of CR: 2 |
| McG: | Discrim Score: 9.90 |
| GvH: | Signal Score (−3.5): 2.04 |
| | Possible site: 20 |

>>> Seems to have a cleavable N-term signal seq.
Amino Acid Composition: calculated from 21
involving clv.sig in the ALOMREC or not: 1B

| ALOM program | | count: 6 value: −11.15 threshold: 0.5 | |
|---|---|---|---|
| | INTEGRAL | Likelihood = | Transmembrane |
| | | −11.15 | 324-340 (313-348) |
| | INTEGRAL | Likelihood = | Transmembrane |
| | | −5.26 | 346-362 (342-369) |
| | INTEGRAL | Likelihood = | Transmembrane |
| | | −4.73 | 681-697 (679-701) |
| | INTEGRAL | Likelihood = | Transmembrane |
| | | −2.44 | 725-741 (723-742) |
| | INTEGRAL | Likelihood = | Transmembrane |
| | | −1.97 | 656-672 (656-673) |
| | INTEGRAL | Likelihood = | Transmembrane |
| | | −1.59 | 422-438 (421-438) |

| | |
|---|---|
| PERIPHERAL | Likelihood = 3.45 |

modified ALOM score: 2.83

>>> Likely a Type IIIa membrane protein (clv)

| | |
|---|---|
| Gavel: | Bound.Mitoch.Preseq. R-2 motif: 5 ARKLSV |
| | mtdisc (mit) Status: positive (3.31) |

*** Reasoning Step: 3

| | |
|---|---|
| KDEL | Count: 0 |

2nd signal for mitochondria (apolar)(5)

from: 5 to: 17 Score: 8.5

SKL motif: pos: 399(752), count: 3 SHL
pox modified by SKL scr: 0.1

| | |
|---|---|
| Poxaac | Score: 2.67 |
| | aac not from the N-term. scr modified |

>>> POX Status: notclr
>>> lys: −1.27 Status: negative
Nuc-4 pos: 443 (5) KKKK
>>> Nuclear Signal. Status: negative (0.00)

A search of the NOV53a protein against the Geneseq and public databases, a proprietary database that contains sequences published in patents and patent publication, yielded several homologous proteins shown in Table 53C. In a BLAST search of public sequence databases, the NOV53a protein was found to have homology to the proteins shown in the BLASTP data in Table 53D.

TABLE 53C
Geneseq and Public Results for NOV53a

| | | NOV53a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAM51198 | Human breast cancer 4 gene | 32 . . . 773 | 735/742 (99%) | |
| | (BCR4)-encoded | 1 . . . 736 | 736/742 (99%) | |
| Q13433 | ESTROGEN REGULATED LIV-1 | 32 . . . 786 | 748/755 (99%) | |
| | PROTEIN. | 1 . . . 749 | 749/755 (99%) | |
| AAE07205 | Human LIV-1 protein | 32 . . . 773 | 735/742 (99%) | |
| | | 1 . . . 736 | 736/742 (99%) | |

PFam analysis predicts that the NOV53a protein contains the domains shown in the Table 53E.

TABLE 53E
Domain Analysis of NOV53a

| | | Score for | |
|---|---|---|---|
| | NOV53a Match | the Matched | Expect |
| Pfam Domain | Region | Region | Value |
| Peptidase_C4 | 251 . . . 266 | 4.6 | 3.4e−56 |
| Osteopontin | 4 . . . 300 | −85.1 | 0.95 |

### Example 54

The NOV54 clone was analyzed, and the nucleotide and encoded polypeptide sequences are shown in Table 54A.

TABLE 54A
NOV54 Sequence Analysis

| | | |
|---|---|---|
| +TL,1NOV54a, CG59905-01 | SEQ ID NO:1109 | 3145 bp |
| DNA Sequence | ORF Start: ATG at 34 | ORF Stop: TGA at 3046 |

ATTTTACTTGCTGGATCTTCAGCCTTGACCTGTATGGCAAATGGCTTATGGGACCGATCCCTGCCCAA
GTGTTTGGCTATATCCTGTGGACACCCAGGGGTCCCTGCCAACGCCGTCCTCACTGGAGAGCTGTTTA
CCTATGGCGCCGTCGTGCACTACTCCTGCAGAGGGAGCGAGAGCCTCATAGGCAACGACACGAGAGTG
TGCCAGGAAGACAGTCACTGGAGCGGGGCACTGCCCCACTGCACAGGAAATAATCCTGGATTCTGTGG
TGATCCGGGGACCCCAGCACATGGGTCTCGGCTTGGTGATGACTTTAAGACAAAGAGTCTTCTCCGCT
TCTCCTGTGAAATGGGGCACCAGCTGAGGGGCTCCCCTGAACGCACGTGTTTGCTCAATGGGTCATGG
TCAGGACTGCAGCCGGTGTGTGAGGCCGTGTCCTGTGGCAACCCTGGCACACCCACCAACGGAATGAT
TGTCAGTAGTGATGGCATTCTGTTCTCCAGCTCGGTCATCTATGCCTGCTGGGAAGGCTACAAGACCT
CAGGGCTCATGACACGGCATTGCACAGCCAATGGGACCTGGACAGGCACTGCTCCCGACTGCACAATT
ATAAGTTGTGGGGATCCAGGCACACTAGCAAATGGCATCCAGTTTGGGACCGACTTCACCTTCAACAA
GACTGTGAGCTATCAGTGTAACCCAGGCTATGTCATGGAAGCAGTCACATCCGCCACTATTCGCTGTA
CCAAAGACGGCAGGTGGAATCCGAGCAAACCTGTCTGCAAAGCCGTGCTGTGTCCTCAGCCGCCGCCG
GTGCAGAATGGAACAGTGGAGGGAAGTGATTTCCGCTGGGGCTCCAGCATAAGTTACAGCTGCATGGA
CGGTTACCAGCTCTCTCACTCCGCCATCCTCTCCTGTGAAGGTCGCGGGGTGTGGAAAGGAGAGATCC
CCAGTGTCTGCCTGTGTTCTGCGGAGACCCTGGCATCCCCGCAGAAGGGCGACTTAGTGGGAAAAGTT
TCACCTATAAGTCCGAAGTCTTCTTCCAGTGCAAATCTCCATTTATACTCGTGGGATCCTCCAGAAGA
GTCTGCCAAGCTGACGGCACGTGGAGCGGCATACAACCCACCTGCATTGGTAATAATTATCATACAGC
TCTGGGGATACCTGGGAGTATTTGGAGATGAGGACGCTTCATTCCGAAATTGGGTCATTTGTGATTAC
ATAGAAAGTGTTTCCATAGACAGTTTTCTGTACAAGGTTGGAAGCACGGTTTTTTTCAGGTGCAGAAA
AGGCTACCATATTCAAGGTTCCACGACTCGCACCTGCCTTGCCAATTTAACATGGAGTGGGATACAGA
CCGAATGTATACCTCATGCCTGCAGACAGCCAGAAACCCCGGCACACGCGGATGTGAGAGCCATCGAT
CTTCCTACTTTCGGCTACACCTTAGTGTACACCTGCCATCCAGGCTTTTTCCTCGCAGGGGGATCTGA
GCACAGAACATGTAAAGCAGACATGAAATGGACAGGAAAGTCGCCTGTGTGTAAAATTCCTTCAGATG
TCTTTTTCGTCAATTCACTGTGGAAGGGGTATTATGAATATTTAGGGAAAAGACAACCCGCCACTCTA
ACTGTTGACTGGTTCAATGCAACAAGCAGTAAGGTGAATGCCACCTTCAGCGAAGCCTCGCCAGTGGA
GCTGAAGTTGACAGGCATTTACAAGAAGGAGGAGGCCCACTTACTCCTGAAAGCTTTTCAAATTAAAG
GCCAGGCAGATATTTTTGTAAGCAAGTTCGAAAATGACAACTGGGGACTAGATGGTTATGTGTCATCT
GGACTTGAAAGAGGAGGATTTACTTTTCAAGGTGACATTCATGGAAAAGACTTTGGAAAATTTAAGCT
AGAAAGGCAAGGATGGGTCACAATATTCTTGAGCCTATTTCTTCATCTTAAATCTCAGTATAGAAGTT
CCCAAGGTTGTTACGAGATTGAGAGGCCACATCCTTTAAACCCAGATCAAGACTCTTCCAGTCATTAC
CACGGCACCAGCAGTGGCTCTGTGGCGGCTGCCATTCTGGTTCCTTTCTTTGCTCTAATTTTATCAGG
GTTTGCATTTTACCTCTACAAACACAGAACGAGACCAAAAGTTCAATACAATGGCTATGCTGGGCATG
AAAACAGCAATGGACAAGCATCGTTTGAAAACCCCATGTATGATACAAACTTAAAACCCACAGAAGCC
AAGGCTAAAACCACACACGGGCTGCTCACGTCTCTGGACTCCAAGAGAAAGATGGTTATAACCCTGGC
AGAGTCTTTACCCGCCCTGCGAACCTGCCTGTTGATCTGCTTCCCACTCCTCTTTAGACTTGCTCGTG
AATCTTCAGCACAGCCATTTGTTTTGAGTATTCAAACACATAAAAAGATATCTGGCAACTTCCCACTT
CTTATGACTTCTCAGACACCTTGTAACGGAGCTTCCCTTGGAGGTGGACAAACTTCTCACTCACAAGA
GAGACGGAGAGCAACAGAGAGAGATGGGCAGGGATTGATTCGAGGATTGGCTGACTCGATTATGGAGG
GTGAGAAGTCCCAGGACAGGCCGTCTGCAAGGCGTAAACCCAGGGAAGCTGCTGGTATGGCTCGGTTC
AAGTCCAAAGGCCTCAGCACCAAGGAAACCAAGGTGATAACTCTCAGTTCGGGTCAAAGTCCTGGGAG
TCTGCAAGGCCTCTCATTGAGTAAGTCTCACAAGATCCGATGGTTTTATAAAGGGCAGTTCCCCTGCA
CAAGCTCTCTTCTCTGCCACCACGCGTTTGCTGTTCATTCACCTTTCACCATGATTGTGAGGCCTCCC
AGCCATGTGGAACTGTGCAAAGAGGATGTTCAAAGGGCATCTTGGACCAGGAGGAGGAAGCCCATTGT
GAGGATGGCAAAGAACAATAAATGGAAGGCCCGTGGAACCCTGAGCGGCTGCCCCATCACTGTCCTCT
CCATCCTTTCCATGCTTCCCATGGTGCATGGCTCTCAGGCTCTCCAAGCACCATGAGCTCCTGGCGCG
TATGGCCTGGCCCCTGAGTGTGGAGTCTGTGGACTTTAAATGTGCCTTTACTCAGCCAGCATCTCCCT
GCTGGGTGCGATGTTG

| | | | |
|---|---|---|---|
| NOV54a, CG59905-01 | SEQ ID NO:1110 | 1004 aa | MW at 109752.7 kD |
| Protein Sequence | | | |

MANGLWDRSLPKCLAISCGHPGVPANAVLTGELFTYGAVVHYSCRGSESLIGNDTRVCQEDSHWSGAL
PHCTGNNPGFCGDPGTPAHGSRLGDDFKTKSLLRFSCEMGHQLRGSPERTCLLNGSWSGLQPVCEAVS
CGNPGTPTNGMIVSSDGTLFSSSVTYACWEGYKTSGLMTRHCTANGTWTGTAPDCTIISCGDPGTLAN
GIQFGTDFTFNKTVSYQCNPGYVMEAVTSATIRCTKDGRWNPSKPVCKAVLCPQPPPVQNGTVEGSDF
RWGSSISYSCMDGYQLSHSAILSCEGRGVWKGEIPSVCLCSAETLASPQKGDLVGKVSPISPKSSSSA
NLHLYSWDPPEESAKLTARGAAYNPPALVIIIIQLWGYLGVFGDEDASFRNWVICDYIESVSIDSFLY
KVGSTVFFRCRKGYEIQGSTTRTCLANLTWSGIQTECIPHACRQPETPAHADVRAIDLPTFGYTLVYT
CHPGFFLAGGSEHRTCKADMKWTGKSPVCKIPSDVFFVNSLWKGYYEYLGKRQPATLTVDWFNATSSK
VNATFSEASPVELKLTGIYKKEEAHLLLKAFQIKGQADIFVSKFENDNWGLDGYVSSGLERGGFTFQG
DIHGKDFGKFKLERQGWVTIFLSLFLHLKSQYRSSQGCYEIERPHPLNPDQDSSSHYHGTSSGSVAAA
ILVPFFALILSGFAFYLYKHRTRPKVQYNGYAGHENSNGQASFENPMYDTNLKPTEAKAKTTHGLLTS
LDSKRKMVITLAESLPALRTCLLICFPLLFRLARESSAQPFVLSIQTHKKISGNFPLLMTSQTPCNGA
SLGGGQTSHSQERRRATERDGQGLIRGLADSIMEGEKSQDRPSARRKPREAAGMARFKSKGLSTKETK
VITLSSGQSPGSLQGLSLSKSHKIRWFYKGQFPCTSSLLCHHAFAVHSPFTMIVRPPSHVELCKEDVQ
RASWTRRRKPIVRMAKNNKWKARGTLSGCPITVLSTLSMLPMVHGSQALQAP

| | | |
|---|---|---|
| NOV54b, CG59905-03 | SEQ ID NO:1111 | 10989 bp |
| DNA Sequence | ORF Start: ATG at 286 | ORF Stop: TGA at 10987 |

CCTTCCTGCCCGGCTCGGCCGGCGCTCGTCCCCGGCCCCGGCCCCGCCAGCCCGGGTCTCCGCGCTCG
GAGCAGCTCAGCCCTGCAGTGGCTCGGGACCCGATGCTATGAGAGGGAAGCGAGCCGGGCGCCCAGAC
CTTCAGGAGGCGTCGGATGCGCGGCGGGTCTTGGGACCGGGCTCTCTCTCCGGCTCGCCTTGCCCTCG
GGTGATTATTTGGCTCCGCTCATAGCCCTGCCTTCCTCGGAGGAGCCATCGGTGTCGCGTGCGTGTGG
AGTATCTGCAGACATGACTGCGTGGAGGAGATTCCAGTCGCTGCTCCTGCTTCTCGGGCTGCTGGTGC
TGTGCGCGAGGCTCCTCACTGCAGCGAAGGGTCAGAACTGTGGAGGCTTAGTCCAGGGTCCCAATGGC
ACTATTGAGAGCCCAGGGTTTCCTCACGGGTATCCGAACTATGCCAACTGCACCTGGATCATCATCAC
GGGCGAGCGCAATAGGATACAGTTGTCCTTCCATACCTTTGCTCTTGAAGAAAATTTTGATATTTTAT
CAGTTTACGATGGACAGCCTCAACAAGGGAATTTAAAAGTGAGATTATCGGGATTTCAGCTGCCCTCC
TCTATAGTGAGTACAGGATCTATCCTCACTCTGTGGTTCACGACAGACTTCGCTGTGAGTGCCCAAGG
TTTCAAAGCATTATATGAAGTTTTACCTAGCCACACTTGTGGAAATCCTGGAGAAATCCTGAAAGGAG
TTCTGCATGGAACGAGATTCAACATAGGAGACAAAATCCGGTACAGCTGCCTCCCTGGCTACATCTTG
GAAGGCCACGCCATCCTGACCTGCATCGTCAGCCCAGGAAATGGTGCATCGTGGGACTTCCCAGCTCC
CTTTTGCAGAGCTGAGGGAGCCTGCGGAGGAACCTTACGCGGGACCAGCAGCTCCATCTCCAGCCCGC
ACTTCCCTTCAGAGTACGAGAACAACGCGGACTGCACCTGGACCATTCTGGCTGAGCCCGGGGACACC
ATTGCGCTGGTCTTCACTGACTTTCAGCTAGAAGAAGGATATGATTTCTTAGAGATCAGTGGCACGGA
AGCTCCATCCATATGGCTAACTGGCATGAACCTCCCCTCTCCAGTTATCAGTAGCAAGAATTGGCTAC
GACTCCATTTCACCTCTGACAGCAACCACCGACGCALAGGATTTAACGCTCAGTTCCAAGTGAAAAAG
GCGATTGAGTTGAAGTCAAGAGGAGTCAAGATGCTGCCCAGCAAGGATGGAAGCCATAAAAACTCTGT
CTTGAGCCAAGGAGGTGTTGCATTGGTCTCTCACATGTGTCTAGATCCTGGGATTCCAGAAAATGGTA
GAAGAGCAGGTTCCGACTTCAGTAGGGTTGGTGCAAATGTACAGTTTTCATGTGAGGACAATTACGTG
CTCCAGGGATCTAAAAGCATCACCTGTCAGAGAGTTACAGAGACGCTCGCTGCTTGGAGTGACCACAG
GCCCATCTGCCGAGCGAGAACATGTGGATCCAATCTGCGTGGGCCCAGCGGCGTCATTACCTCCCCTA
ATTATCCGGTTCAGTATGAAGATAATGCACACTGTGTGTGGGTCATCACCACCACCGACCCGGACAAG
GTCATCAAGCTTGCCTTTGAAGAGTTTGAGCTGGAGCGAGGCTATGACACCCTGACGGTTGGTGATGC
TGGGAAGGTGGGAGACACCAGATCGGTCTTGTACGTGCTCACGGGATCCAGTGTTCCTGACCTCATTG
TGAGCATGAGCAACCAGATGTGGCTACATCTGCAGTCGGATGATAGCATTGGCTCACCTGGGTTTAAA
GCTGTTTACCAAGAAATTGAAAAGGGAGGGTGTGGGGATCCTGGAATCCCCGCCTATGGGAAGCGGAC
GGGCAGCAGTTTCCTCCATGGAGATACACTCACCTTTGAATGCCCGGCGGCCTTTGAGCTGGTGGGGG
AGAGAGTTATCACCTGTCAGCAGAACAATCAGTGGTCTGGCAACAAGCCCAGCTGTGTATTTTCATGT
TTCTTCAACTTTACGGCATCATCTGGGATTATTCTGTCACCAAATTATCCAGAGGAATATGGGAACAA
CATGAACTGTGTCTGGTTGATTATCTCGGAGCCAGGAAGTCGAATTCACCTAATCTTTAATGATTTTG
ATGTTGAGCCTCAATTTGACTTTCTCGCGGTCAAGGATAATGGCATTTCTGACATAACTGTCCTGGGT
ACTTTTTCTGGCAATGAAGTGCCTTCCCAGCTGGCCAGCAGTGGGCATATAGTTCGCTTGGAATTTCA
GTCTGACCATTCCACTACTGGCAGAGGGTTCAACATCACTTACACCACATTTGGTCAGAATGAGTGCC
ATGATCCTGGCATTCCTATAAACGGACGACGTTTTGGTGACAGGTTTCTACTCGGGAGCTCGGTTTCT
TTCCACTGTGATGATGGCTTTGTCAAGACCCAGGGATCCGAGTCCATTACCTGCATACTGCAAGACGG
GAACGTGGTCTGGAGCTCCACCGTGCCCCGCTGTGAAGCTCCATGTGGTGGACATCTGACAGCGTCCA
GCGGAGTCATTTTGCCTCCTGGATGGCCAGGATATTATAAGGATTCTTTACATTGTGAATGGATAATT
GAAGCAAAACCAGGCCACTCTATCAAAATGACTTTTGACAGATTTCAGACAGAGGTCAATTATGACAC
CCCAGTTCCTCATCAGCACCGGGAACTTCATGTACCTGCTGTTCACCACTGACAACAGCCGCTCCAGC
ATCGGCTTCCTCATCCACTATGAGAGTGTGACGCTTGAGTCGGATTCCTGCCTGGACCCGGGCATCCC
TGTGAACGGCCATCGCCACGGTGGAGACTTTGGCATCAGGTCCACAGTGACTTTCAGCTGTGACCCGG
GGTACACACTAAGTGACGACGAGCCCCTCGTCTGTGAGAGGAACCACCAGTGGAACCACGCCTTGCCC
AGCTGCGACGCTCTATGTGGAGGCTACATCCAAGGGAAGAGTGGAACAGTCCTTTCTCCTGGGTTTCC
AGATTTTTATCCAAACTCTCTAAACTGCACGTGGACCATTGAAGTGTCTCATGGGAAAGGAGTTCAAA
TGCTCTTTCACACCTTTCATCTTGAGAGTTCCCACGACTATTTACTGATCACAGAGGATGGAAGTTTT
TCCGAGCCCGTTGCCAGGCTCACCGGGTCGGTGTTGCCTCATACGATCAAGGCAGGCCTGTTTGGAAA
CTTCACTGCCCAGCTTCGGTTTATATCAGACTTCTCAATTTCGTACGAGGGCTTCAATATCACATTTT
CAGAATATGACCTGGAGCCATGTGATGATCCTGGAGTCCCTGCCTTCAGCCGAAGAATTGGTTTTCAC
TTTGGTGTGGGAGACTCTCTGACGTTTTCCTGCTTCCTGGGATATCGTTTAGAAGGTGCCACCAAGCT
TACCTGCCTGGGTGGGGGCCGCCGTGTGTGGAGTGCACCTCTGCCAAGGTGTGTGGCCGAATGTGGAG
CAAGTGTCAAAGGAAATGAAGGAACATTACTGTCTCCAAATTTTCCATCCAATTATGATAATACCCAT
GAGTGTATCTATAAAATAGAAACAGAAGCCGGCAAGGGCATCCACCTTAGAACACGAAGCTTCCAGCT
GTTTGAAGGAGATACTCTAAAGGTATATGATGGAAAAGACAGTTCCTCACGTCCACTGGGCACGTTCA
CTAAAAATGAACTTCTGGGGCTGATCCTAAACAGCACATCCAATCACCTATGGCTAGAGTTCAACACC
AATGGATCTGACACCGACCAAGGTTTTCAACTCACCTATACCAGTTTTGATCTGGTAAAATGTGAGGA
TCCGGGCATCCCTAACTACGGCTATAGGATCCGTGATGAAGGCCACTTTACCGACACTGTAGTTCTGT
ACAGTTGCAACCCGGGGTACGCCATGCATGGCAGCAACACCCTGACCTGTTTGAGTGGAGACAGGAGA
GTGTGGGACAAACCACTACCTTCGTGCATAGCGGAATGTGGTGGTCAGATCCATGCAGCCACATCAGG
ACGAATATTGTCCCCTGGCTATCCAGCTCCGTATGACAACAACCTCCACTGCACCTGGATTATAGAGG
CAGACCCAGGAAAGACCATTAGCCTCCATTTCATTGTTTTCGACACGGAGATGGCTCACGACATCCTC
AAGGTCTGGGACGGGCCGGTGGACAGTGACATCCTGCTGAAGGAGTGGAGTGGCTCCGCCCTTCCGGA
GGACATCCACAGCACCTTCAACTCACTCACCCTGCAGTTCGACAGCGACTTCTTCATCAGCAAGTCTG
GCTTCTCCATCCAGTTCTCCACCTCAATTGCAGCCACCTGTAACGATCCAGGTATGCCCCAAAATGGC
ACCCGCTATGGAGACAGCAGAGAGGCTGGAGACACCGTCACATTCCAGTGTGACCCTGGCTATCAGCT
CCAAGGACAAGCCAAAATCACCTGTGTGCAGCTGAATAACCGGTTCTTTTGGCAACCAGACCCTCCTA
CATGCATAGCTGCTTGTGGAGGGAATCTGACGGGCCCAGCAGGTGTTATTTTGTCACCCAACTACCCA
CAGCCGTATCCTCCTGGGAAGGAATGTGACTGGAGAGTAAAAGTGAACCCGGACTTTGTCATCGCCTT
GATATTCAAAAGTTTCAACATGGAGCCCAGCTATGACTTCCTACACATCTATGAAGGGGAAGATTCCA
ACAGCCCCCTCATTGGGAGTTACCAGGGCTCTCAGGCCCCAGAAAGAATAGAGAGTAGCGGAAACAGC
CTGTTTCTGGCATTTCGGAGTGATGCCTCCGTGGGCCTTTCAGGGTTCGCCATTGAATTTAAAGAGAA
ACCACGGGAAGCTTGTTTTGACCCAGGAAATATAATGAATGGGACAAGAGTTGGAACAGACTTCAAGC
TTGGCTCCACCATCACCTACCAGTGTGACTCTGGCTATAAGATTCTTGACCCCTCATCCATCACCTGT
GTGATTGGGGCTGATGGGAAACCCTCCTGGGACCAAGTGCTGCCCTCCTGCAATGCTCCCTGTGGAGG
CCAGTACACGGGATCAGAAGGGGTAGTTTTATCACCAAACTACCCCCATAATTACACAGCTGGTCAAA
TATGCCTCTATTCCATCACGGTACCAAAGGAATTCGTGGTCTTTGGACAGTTTGCCTATTTCCAGACA
GCCCTGAATGATTTGGCAGAATTATTTGATGGAACCCATGCACAGGCCAGACTTCTCAGCTCACTCTC
GGGGTCTCACTCAGGGGAAACATTGCCCTTGGCTACGTCAAATCAAATTCTGCTCCGATTCAGTGCAA
AGAGCGGTGCCTCTGCCCGCGGCTTCCACTTCGTGTATCAAGCTGTTCCTCGTACCAGTGACACCCAA
TGCAGCTCTGTCCCCGAGCCCAGATACGGAAGGAGAATTGGTTCTGAGTTTTCTGCCGGCTCCATCGT
CCGATTCGAGTGCAACCCGGGATACCTGCTTCAGGGTTCCACGGCGCTCCACTGCCAGTCCGTGCCCA
ACGCCTTGGCACAGTGGAACGACACGATCCCCAGCTGTGTGGTACCCTGCAGTGGCAATTTCACTCAA
CGAAGAGGTACAATCCTGTCCCCCGGCTACCCTGAGCCATACGGAAACAACTTGAACTGTATATGGAA
GATCATAGTTACGGAGGGCTCGGGAATTCAAGATCCAAGTGATCAGTTTTGCCACGGAGCAGAACTGG
GACTTCCCTTTGAGATCCACGATGGTGGGGATGTGACCGCACCCAGACTGGGAAGCTTCTCAGGCACC
ACAGTACCGGCACTGCTGAACAGTACTTCCAACCAACTCTACCTGCATTTCCAGTCTGACATTAGTGT
GGCAGCTGCTGGTTTCCACCTGGAATACAAAACTGTAGGTCTTGCTGCATGCCAAGAACCAGCCCTCC
CCAGCAACAGCATCAAAATCGGAGATCGGTACATGGTGAACGACGTGCTCTCCTTCCAGTGCGAGCCC
GGGTACACCCTGCAGGGCCGTTCCCACATTTCCTGTATGCCAGGGACCGTTCGCCGTTGGAACTATCC
GTCTCCCCTGTGCATTGCAACCTGTGGAGGGACGCTGAGCACCTTGGGTGGTGTGATCCTGAGCCCCG
GCTTCCCAGGTTCTTACCCCAACAACTTAGACTGCACCTGGAGGATCTCATTACCCATCGGCTATGGT
GCACATATTCAGTTTCTGAATTTTTCTACCGAAGCTAATCATGACTTCCTTGAAATTCAAAATGGACC
TTACCACACCAGCCCCATGATTGGACAATTTAGCGGCACGGATCTCCCCGCGGCCCTGCTGAGCACAA
CGCATGAAACCCTCATCCACTTTTATAGTGACCATTCGCAAAACCGGCAAGGATTTAAACTTGCTTAC
CAAGCCTATGAATTACAGAACTGTCCAGATCCACCCCCATTTCAGAATGGGTACATGATCAACTCGGA
TTACAGCGTGGGGCAATCAGTATCTTTCGAGTGTTATCCTGGGTACATTCTAATAGGCCATCCTGTCC
CCACTTGTCAGCATGGGATCAACAGAAACTGGAACTACCCTTTTCCAAGATGTGATGCCCCTTGTGGG
TACAACGTAACTTCTCAGAACGGCACCATCTACTCCCCTGGCTTTCCTGATGAGTATCCGATCCTGAA
GGACTGCATTTGGCTCATCACGGTGCCTCCAGGGCACGGAGTTTACATCAACTTCACCCTGTTACAGA
TTCAGTGGCAACACAGCCCTCGAAACGGCGTATAGCTCCACCAACCAAGTCCTGCTCAAGTTCCACAG
CGACTTTTCAAATGGAGGCTTCTTTGTCCTCAATTTCCACGCATTTCAGCTCAAGAAATGTCAACCTC
CCCCAGCGGTTCCACAGGCAGAAATGCTTACTGAGGATGATGATTTCGAAATAGGAGATTTTGTGAAG
TACCAGTGCCACCCCGGGTACACCTTGGTGGGGACCGACATTCTGACTTGCAAGCTCAGTTCCCAGTT
GCAGTTTGAGGGTTCTCTCCCAACATGTGAAGCACAATGCCCAGCAAATGAAGTCCGGACTGGATCAT
CGGGAGTCATTCTCAGTCCAGGGTATCCGGGTAATTATTTTAACTCCCAGACTTGCTCTTGGAGTATT
ACTGGAAGTGTTTGATGGTTCTTCTGGGCAAAGTCCTCTGCTAGTAGTCTTAAGTGGGAATCATACTG
AACAATCAAATTTTACAAGCAGGAGTAATCAGTTATATCTCCGCTGGTCCACTGACCATGCCACCAGT
AAGAAAGGATTCAAGATTCGCTATGCAGCACCTTACTGCAGTTTGACCCACCCCCTGAAGAATGGGGG
TATTCTAAACAGGACTGCAGGAGCGGTTGGAAGCAAAGTGCATTATTTTTGCAAGCCTGGATACCGAA
TGGTCGGCCACAGCAATGCAACCTGTAGACGAAACCCACTTGGCATGTACCAGTGGGACTCCCTCACG
CCACTCTGCCAGGCTGTGTCCTGTGGAATCCCAGAATCCCCAGGAAACGGTTCATTTACCGGGAACGA
GTTCACTTTGGACAGTAAAGTGGTCTATGAATGTCATGAAGGCTTCAAGCTTGAATCCAGCCAGCAAG
CAACAGCCGTGTGTCM~GAAGATGGGCTGTGGAGTAACAAGGGGAAGCCGCCCATGTGTAAGCCGGTC
GCTTGCCCCAGCATTGAAGCTCAGCTCTCAGAACATGTCATCTGGAGGCTGGTTTCAGGATCCTTGAA
TGAGTACGGTGCTCAAGTATTGCTGAGCTGCAGTCCTGGTTACTACTTAGAAGGCTGGAGGCTCCTGC
GGTGCCAGGCCAATGGGACGTGGAACATAGGAGATGAGAGGCCAAGCTGTCGAGTTATCTCGTGTGGA
AGCCTTTCCTTTCCCCCAAATGGCAACAAGATTGGAACGTTGACAGTTTATGGGGCCACAGCTATATT
TACGTGCAACACCGGCTACACGCTTGTGGGGTCTCATGTCAGAGAGTGCTTGGCAAATGGGCTCTGGA
GCGGCAGCGAAACTCGATGTTTAGCTGGCCACTGCGGTTCCCCAGACCCGATTGTGAACGGTCACATT
AGTGGAGATGGCTTCAGTTACAGAGACACGGTGGTTTACCAGTGCAATCCTGGTTTCCGGCTTGTGGG
AACTTCCGTGAGGATATGCCTGCAAGACCACAAGTGGTCTGGACAAACGCCTGTCTGTGTCCGTATCA
CATGTGGTCACCCTGGAAACCCTGCCCACGGATTCACTAATGGCAGTGAGTTCAACCTGAATGATGTC
GTGAATTTCACCTGCAACACGGGCTATTTGCTGCAGGGCGTGTCTCGAGCCCAGTGTCGGAGCAACGG
CCAGTGGAGTAGCCCTCTGCCCACGTGTCGAGTGGTGAACTGTTCTGATCCAGGCTTTGTGGAAAATG
CCATTCGTCACGGGCAACAGAACTTCCCTGAGAGTTTTGAGTATGGAATGAGTATCCTGTACCATTGC
AAGAAGGGATTTTACTTGCTGGGATCTTCAGCCTTGACCTGTATGGCAAATGGCTTATGGGACCGATC
CCTGCCCAAGTGTTTGGCTATATCGTGTGGACACCCAGGGGTCCCTGCCAACGCCGTCCTCACTGGAG
AGCTGTTTACCTATGGCGCCGTCGTGCACTACTCCTGCAGAGGGAGCGAGAGCCTCATAGGCAACGAC
ACGAGAGTGTGCCAGGAAGACAGTCACTGGAGCGGGGCACTGCCCCACTGCACAGGAAATAATCCTGG
ATTCTGTGGTGATCCGGGGACCCCAGCACATGGGTCTCGGCTTGGTGATGACTTTAAGACAAAGAGTC
TTCTCCGCTTCTCCTGTGAAATGGGGCACCAGCTGAGGGGCTCCCCTGAACGCACGTGTTTGCTCAAT
GGGTCATGGTCAGGACTGCAGCCGGTGTGTGAGGCCGTGTCCTGTGGCAACCCTGGCACACCCACCAA
CGGAATGATTGTCAGTAGTGATGGCATTCTGTTCTCCAGCTCGGTCATCTATGCCTGCTGGGAAGGCT
ACAAGACCTCAGGGCTCATGACACGGCATTGCACAGCCAATGGGACCTGGACAGGCACTGCTCCCGAC
CTTCAACAAGACTGTGAGCTATCAGTGTAACCCAGGCTATGTCATGGAAGCAGTCACATCCGCCACTA
TTCGCTGTACCAAAGACGGCAGGTGGAATCCGAGCAAACCTGTCTGCAAAGCCGTGCTGTGTCCTCAG
CCGCCGCCGGTGCAGAATGGAACAGTGGAGGGAAGTGATTTCCGCTGGGGCTCCAGCATAAGTTACAG
CTGCATGGACGGTTACCAGCTCTCTCACTCCGCCATCCTCTCCTGTGAAGGTCGCGGGGTGTGGAAAG
GAGAGATCCCCCAGTGTCTCGCTGTGTTCTGCGGAGACCCTGGCATCCCCGCAGAAGGGCGACTTAGT
GGGAAAAGTTTCACCTATAAGTCCGAAGTCTTCTTCCAGTGCAAATCTCCATTTATACTCGTGGGATC
CTCCAGAAGAGTCTGCCAAGCTGACGGCACGTGGAGCGGCATACAACCCACCTGCATTGGTGATCCTG
CTCATAACACCTGCCCAGACCCTGGTACGCCACACTTTGGAATACAGAATAGCTCCAGAGGCTATGAG
GTTGGAAGCACGGTTTTTTTCAGGTGCAGAAAAGGCTACCATATTCAAGGTTCCACGACTCGCACCTG
CCTTGCCAATTTAACATGGAGTGGGATACAGACCGAATGTATACGTCATGCCTGCAGACAGCCAGAAA
CCCCGGCACACGCGGATGTGAGAGCCATCGATCTTCCTACTTTCGGCTACACCTTAGTGTACACCTGC
CATCCAGGCTTTTTCCTCGCAGGGGGATCTGAGCACAGAACATGTAAAGCAGACATGAAATGGACAGG
CTTCAGATGTCTTTTTCGTCAATTCACTGTGGAAGGGGTATTATGAATATTTAGGGAAAAGACAACCC
GCCACTCTAACTGTTGACTGGTTCAATGCAACAAGCAGTAAGGTGAATGCCACCTTCAGCGAAGCCTC
GCCAGTGGAGCTGAAGTTGACAGGCATTTACAAGAAGGAGGAGGCCCACTTACTCCTGAAAGCTTTTC
AAATTAAAGGCCAGGCAGATATTTTTGTAAGCAAGTTCGAAAATGACAACTGGGGACTAGATGGTTAT
ATTTAAGCTAGAAAGGCAAGATCCTTTAAACCCAGATCAAGACTCTTCCAGTCATTACCACGGCACCA
GCAGTGGCTCTGTGGCGGCTGCCATTCTGGTTCCTTTCTTTGCTCTAATTTTATCAGGGTTTGCATTT
TACCTCTACAAACACAGAACGAGACCAAAAGTTCAATACAATGGCTATGCTGGGCATGAAAACAGCAA
TGGACAAGCATCGTTTGAAAACCCCATGTATGATACAAACTTAAAACCCACAGAAGCCAAGGCTGTGA
GGTTTGACACAAGTGTGAACACAGTCTGTACAGTGGTATGA

| | | | |
|---|---|---|---|
| NOV54b, CG59905-03 | SEQ ID NO:1112 | 3567 aa | MW at 389007.4 kD |
| Protein Sequence | | | |

MTAWRRFQSLLLLLGLLVLCARLLTAAKGQNCGGLVQGPNGTIESPGFPHGYPNYANCTWIIITGERN
RIQLSFHTFALEENFDILSVYDGQPQQGNLKVRLSGFQLPSSIVSTGSILTLWFTTDFAVSAQGFKAL
YEVLPSHTCGNPGEILKGVLHGTRFNIGDKIRYSCLPGYILEGHAILTCIVSPGNGASWDFPAPFCRA
EGACGGTLRGTSSSISSPHFPSEYENNADCTWTILAEPGDTIALVFTDFQLEEGYDFLEISGTEAPSI
WLTGMNLPSPVISSKNWLRLHFTSDSNHRRKGFNAQFQVKKAIELKSRGVKMLPSKDGSHKNSVLSQG
GVALVSHMCLDPGIPENGRPAGSDFSRVGANVQFSCEDNYVLQGSKSITCQRVTETLAAWSDHRPICR
ARTCGSNLRGPSGVITSPNYPVQYEDNAHCVWVITTTDPDKVIKLAFEEFELERGYDTLTVGDAGKVG
DTRSVLYVLTGSSVPDLIVSMSNQMWLHLQSDDSIGSPGFKAVYQEIEKGGCGDPGIPAYGKRTGSSF
LHGDTLTFECPAAFELVGERVITCQQNNQWSGNKPSCVFSCFFNFTASSGIILSPNYPEEYGNNMNCV
TTGRGFNITYTTFGQNECHDPGIPINGRRFGDRFLLGSSVSFHCDDGFVKTQGSESITCILQDGNVVW
SSTVPRCEAPCGGHLTASSGVILPPGWPGYYKDSLHCEWIIEAKPGHSIKMTFDRFQTEVNYDTLEVR
DGPASSSPLIGEYHGTQAPQFLISTGNFMYLLFTTDNSRSSIGFLIHYESVTLESDSCLDPGIPVNGH
RHGGDFGIRSTVTFSCDPGYTLSDDEPLVCERNHQWNHALPSCDALCGGYIQGKSGTVLSPGFPDFYP
NSLNCTWTIEVSHGKGVQMIFHTFHLESSHDYLLITEDGSFSEPVARLTGSVLPHTIKAGLFGNFTAQ
LRFISDFSISYEGFNITFSEYDLEPCDDPGVPAFSRRIGFHFGVGDSLTFSCFLGYRLEGATKLTCLG
TLKVYDGKDSSSRPLGTFTKNELLGLILNSTSNHLWLEFNTNGSDTDQGFQLTYTSFDLVKCEDPGIP
NYGYRIRDEGHFTDTVVLYSCNPGYAMHGSNTLTCLSGDRRVWDKPLPSCIAECGGQIHAATSGRILS
PGYPAPYDNNLHCTWIIEADPGKTISLHFIVFDTEMAHDILKVWDGPVDSDILLKEWSGSALPEDIHS
TFNSLTLQFDSDFFISKSGFSIQFSTSIAATCNDPGMPQNGTRYGDSREAGDTVTFQCDPGYQLQGQA
KITCVQLNNRFFWQPDPPTCIAACGGNLTGPAGVILSPNYPQPYPPGKECDWRVKVNPDFVIALIFKS
FNMEPSYDFLHIYEGEDSNSPLIGSYQGSQAPERIESSGNSLFLAFRSDASVGLSGFAIEFKEKPREA
CFDPGNIMNGTRVGTDFKLGSTITYQCDSGYKILDPSSITCVIGADGKPSWDQVLPSCNAPCGGQYTG
SEGVVLSPNYPHNYTAGQICLYSITVPKEFVVFGQFAYFQTALNDLAELFDGTHAQARLLSSLSGSHS
GETLPLATSNQILLRFSAKSGASARGFHFVYQAVPRTSDTQCSSVPEPRYGRRIGSEFSAGSIVRFEC
NPGYLLQGSTALHCQSVPNALAQWNDTIPSCVVPCSGNFTQRRGTILSPGYPEPYGNNLNCIWKIIVT
EGSGIQDPSDQFCHGAELGLPFEIHDGGDVTAPRLGSFSGTTVPALLNSTSNQLYLHFQSDISVAAAG
FHLEYKTVGLAACQEPALPSNSIKIGDRYMVNDVLSFQCEPGYTLQGRSHISCMPGTVRRWNYPSPLC
IATCGGTLSTLGGVILSPGFPGSYPNNLDCTWRISLPIGYGAHIQFLNFSTEANHDFLEIQNGPYHTS
PMIGQFSGTDLPAALLSTTHETLIHFYSDHSQNRQGFKLAYQAYELQNCPDPPPFQNGYMINSDYSVG
QSVSFECYPGYILIGHPVLTCQHGINRNWNYPFPRCDAPCGYNVTSQNGTIYSPGFPDEYPILKDCIW
LITVPPGHGVYINFTLLQTEAVNDYIAVWDGPDQNSPQLGVFSGNTALETAYSSTNQVLLKFHSDFSN
GGFFVLNFHAFQLKKCQPPPAVPQAEMLTEDDDFEIGDFVKYQCHPGYTLVGTDILTCKLSSQLQFEG
SLPTCEAQCPANEVRTGSSGVILSPGYPGNYFNSQTCSWSIKVEPNYNITIFVDTFQSEKQFDALEVF
DGSSGQSPLLVVLSGNHTEQSNFTSRSNQLYLRWSTDHATSKKGFKIRYAAPYCSLTHPLKNGGILNR
TAGAVGSKVHYFCKPGYRMVGHSNATCRRNPLGMYQWDSLTPLCQAVSCGIPESPGNGSFTGNEFTLD
SKVVYECHEGFKLESSQQATAVCQEDGLWSNKGKPPMCKPVACPSIEAQLSEHVIWRLVSGSLNEYGA
QVLLSCSPGYYLEGWRLLRCQANGTWNIGDERPSCRVISCGSLSFPPNGNKIGTLTVYGATAIFTCNT
GYTLVGSHVRECLANGLWSGSETRCLAGHCGSPDPIVNGHISGDGFSYRDTVVYQCNPGFRLVGTSVR
ICLQDHKWSGQTPVCVRITCGHPGNPAHGFTNGSEFNLNDVVNFTCNTGYLLQGVSRAQCRSNGQWSS
PLPTCRVVNCSDPGFVENAIRHGQQNFPESFEYGMSILYHCKKGFYLLGSSALTCMANGLWDRSLPKC
LAISCGHPGVPANAVLTGELFTYGAVVHYSCRGSESLIGNDTRVCQEDSHWSGALPHCTGNNPGFCGD
PGTPAHGSRLGDDFKTKSLLRFSCEMGHQLRGSPERTCLLNGSWSGLQPVCEAVSCGNPGTPTNGMIV
SSDGILFSSSVIYACWEGYKTSGLMTRHCTANGTWTGTAPDCTVISCGDPGTLANGIQFGTDFTFNKT
VSYQCNPGYVMEAVTSATIRCTKDGRWNPSKPVCKAVLCPQPPPVQNGTVEGSDFRWGSSISYSCMDG
YQLSHSAILSCEGRGVWKGEIPQCLAVFCGDPGIPAEGRLSGKSFTYKSEVFFQCKSPFILVGSSRRV
CQADGTWSGIQPTCIGDPAHNTCPDPGTPHFGIQNSSRGYEVGSTVFFRCRKGYHIQGSTTRTCLANL
TWSGIQTECIRHACRQPETPAHADVRAIDLPTFGYTLVYTCHPGFFLAGGSEHRTCKADMKWTGKSPV
CKSKGVREVNETVTKTPVPSDVFFVNSLWKGYYEYLGKRQPATLTVDWFNATSSKVNATFSEASPVEL
KLTGTYKKEEAHLLLKAFQIKGQADIFVSKFENDNWGLDGYVSSGLERGGFTFQGDIHGKDFGKFKLE
RQDPLNPDQDSSSHYHGTSSGSVAAAILVPFFALILSGFAFYLYKHRTRPKVQYNGYAGHENSNGQAS
FENPMYDTNLKPTEAKAVRFDTTLNTVCTVV

| | | |
|---|---|---|
| NOV54c, 275631102 | SEQ ID NO:1113 | 898 bp |
| DNA Sequence | ORF Start: at 2 | ORF Stop: end of sequence |

CACCAGATCTTGTGGACACCCAGGGGTCCCTGCCAACGCCGTCCTCACTGGAGAGCTGTTTACCTATG
GCGCCGTCGTGCACTACTCCTGCAGAGGGAGCGAGAGCCTCATAGGCAACGACACGAGAGTGTGCCAG
GAAGACAGTCACTGGAGCGGGGCACTGCCCCACTGCACAGGAAATAATCCTGGATTCTGTGGTGATCC
GGGGACCCCAGCACATGGGTCTCGGCTTGGTGATGACTTTAAGACAAAGAGTCTTCTCCGCTTCTCCT
GTGAAATGGGGCACCAGCTGAGGGGCTCCCCTGAACGCACGTGTTTGCTCAATGGGTCATGGTCAGGA
CTGCAGCCGGTGTGTGAGGCCGTGTCCTGTGGCAACCCTGGCACACCCACCAACGGAATGATTGTCAG
TAGTGATGGCATTCTGTTCTCCAGCTCGGTCATCTATGCCTGCTGGGAAGGCTACAAGACCTCAGGGC
TCATGACACGGCATTGCACAGCCAATGGGACCTGGACAGGCACTGCTCCCGACTGCACAATTATAAGT
TGTGGGGATCCAGGCACACTAGCAAATGGCATCCAGTTTGGGACCGACTTCACCTTCAACAAGACTGT
GAGCTATCAGTGTAACCCAGGCTATGTCATGGAAGCAGTCACATCCGCCACTATTCGCTGTACCAAAG
ACGGCAGGTGGAATCCGAGCAAACCTGTCTGCAAAGCCGTGCTGTGTCCTCAGCCGCCGCCGGTGCAG
AATGGAACAGTGGAGGGAAGTGATTTCCGCTGGGGCTCCAGCATAAGTTACAGCTGCATGGACGGTTA
CCAGCTCTCTCACTCCGCCATCCTCTCCTGTGAAGGTCGCGGGGTGTGGAAAGGAGAGATCCCCAGTG
TCTGCCTCGAGGGC

| | | | |
|---|---|---|---|
| NOV54c, 275631102 | SEQ ID NO:1114 | 299 aa | MW at 31648.2 kD |
| Protein Sequence | | | |

TRSCGHPGVPANAVLTGELFTYGAVVHYSCRGSESLIGNDTRVCQEDSHWSGALPHCTGNNPGFCGDP
GTPAHGSRLGDDFKTKSLLRFSCEMGHQLRGSPERTCLLNGSWSGLQPVCEAVSCGNPGTPTNGMIVS
SDGILFSSSVIYACWEGYKTSGLMTRHCTANGTWTGTAPDCTIISCGDPGTLANGIQFGTDFTFNKTV
SYQCNPGYVMEAVTSATIRCTKDGRWNPSKPVCKAVLCPQPPPVQNGTVEGSDFRWGSSISYSCMDGY
QLSHSAILSCEGRGVWKGEIPSVCLEG

| | | |
|---|---|---|
| NOV54d, CG59905-02 | SEQ ID NO:1115 | 3019 bp |
| DNA Sequence | ORF Start: ATG at 184 | ORF Stop: TAA at 2614 |

CTGCCAGGCTGTGTCCTGTGGAATCCCAGAATCCCCAGGAAACGGTTCATTACCGGGAACGAGTTCAC
TTTGGACAGTAAAGTGGTCTATGAATGTCATGAGGGCTTCAAGCTTGAATCCAGCCAGCAAGCAACAG
CCGTGTGTCAAGAAGATGGGCTGTGGAGTAACAAGGGGAAGCCGCCCATGTGTAAGCCGGTCGCTTGC
CCCAGCATTGAAGCTCAGCTCTCAGAACATGTCATCTGGAGGCTGGTTTCAGGATCCTTGAATGAGTA
CGGTGCTCAAGTATTGCTGAGCTGCAGTCCTGGTTACTACTTAGAAGGCTGGAGGCTCCTGCGGTGCC
AGGCCAATGGGACGTGGAGCATAGGAGATGAGAGGCCAAGCTGTCGAGTTATCTCGTGTGGAAGCCTT
TCCTTTCCCCCAAATGGCAACAAGATTGGAACGTTGACAGTTTATGGGGCCACAGCTATATTTACGTG
CAACACCGGCTACACGCTTGTGGGGTCTCATGTCAGAGAGTGCTTGGCAAATGGGCTCTGGAGCGGCA
GCGAAACTCGATGTCTGGCTGGCCACTGCGGTTCCCCAGACCCGATTGTGAACGGTCACATTAGTGGA
GATGGCTTCAGTTACAGAGACACGGTGGTTTACCAGTGCAATCCTGGTTTCCGGCTTGTGGGAACTTC
CGTGAGGATATGCCTGCAAGACCACAAGTGGTCTGGACAAACGCCTGTCTGTGTCGCCATCACATGTG
GTCACCCTGGAAACCCTGCCCACGGATTCACTAATGGCAGTGAGTTCAACCTGAATGATGTCGTGAAT
TTCACCTGCAACACGGGCTATTTGCTGCAGGGCGTGTCTCGAGCCCAGTGTCGGAGCAACGGCCAGTG
GAGTAGCCCTCTGCCCACGTCTCGACACGTGGGCAGAGGGCTACTCCACTGGCGGTTGCTCCGACACG
GGGCTCCGAGACACGCCCTGCAGCAAATAGCCCGTGTTGCAGGTGAAATTCACGACATCATTATTTTA
CTTGCTGGATCTTCAGCCTTGACCTGTATGGCAAATGGCTTATGGGACCGATCCCTGCCCAAGTGTTT
GGCTATATCGTGTGGACACCCAGGGGTCCCTGCCAACGCCGTCCTCACTGGAGAGCTGTTTACCTATG
GCGCCGTCGTGCACTACTCCTGCAGAGGGAGCGAGAGCCTCATAGGCAACGACACGAGAGTGTGCCAG
GAAGACAGTCACTGGAGCGGGGCACTGCCCCACTGCACAGGTAATAATCCTGGATTCTGTGGTGATCC
GGGGACCCCAGCACATGGGTCTCGGCTTGGTGATGACTTTAAGACAAAGAGTCTTCTCCGCTTCTCCT
GTGAAATGGGGCACCAGCTGAGGGGCTCCCCTGAACGCACGTGTTTGCTCAATGGGTCATGGTCAGGA
CTGCAGTCGGTGTGTGAGGCCGTGTCCTGTGGCAACCCTGGCACACCCACCAACGGAATGATTGTCAG
TAGTGATGGCATTCTGTTCTCCAGCTCGGTCATCTATGCCTGCTGGGAAGGCTACAAGACCTCAGGGC
TCATGACACGGCATTGCACAGCCAATGGGACCTGGACAGGCACTGCTCCCGACTGCTTAGTTATAAGT
TGTGGGGATCCAGGCACACTAGCAAATGGCATCCAGTTTGGGACCGACTTCACCTTCAACAAGACTGT
GAGCTATCAGTGTAACCCAGGCTATGTCATGGAAGCAGTCACATCCGCCACTATTCGCTGTACCAAAG
ACGGCAGGTGGAATCCGAGCAAACCTGTCTGCAAAGGTTTGCTGTGTCCTCAGCCGCCGCCGGTGCAG
AATGGAACAGTGGAGGGAAGTGATTTCCGCTGGGGCTCCAGCATAAGTTACAGCTGCATGGACGGTTA
CCAGCTCTCTCACTCCGCCATCCTCTCCTGTGAAGGTCGCGGGGTGTGGAAAGGAGAGATCCCCCAGT
GTCTGCCTGTGTTCTGCGGAGACCCTGGCATCCCCGCAGAGGGGCGACTTAGTGGGAAAAGTTTCACC
TATAAGTCCGAAGTCTTCTTCCAGTGCAAATCTCCATTTATACTCGTGGGATCCTCCAGAAGAGTCTG
CCAAGCTGACGGCACGTGGAGCGGCATACAACCCACCTGCATTGATCCTGCTCATAACACCTGCCCAG
ACCCTGGTACGCCACACTTTGGAATACAGAATAGCTCCAGAGGCTATGAGGTTGGAAGCACGGTTTTT
TTCAGGTGCAGAAAAGGCTACCATATTCAAGGTTCCACGACTCGCACCTGCCTTGCCAATTTAACATG
GAGTGGGATACAGACCGAATGTATACCTCATGCCTGCAGACAGCCAGAAACCCCGGCACACGCGGATG
TGAGAGCCATCGATCTTCCTACTTTCGGCTACACCTTAGTGTACACCTGCCATCCAGGCTTTTTCCTC
GCAGGGGGATCTGAGCACAGAACATGTAAAGCAGACATGAAATGGACAGGAAAGTCGCCTGTGTGTAA
AAGTAAGTCATTGATCAAGGAGTGGCATACTTTTCCAAAGAGCAAGGGAAAGGATCAGCTGCCCATCA
GGCTAGTGTCCACGTGTGAGACCCATAAGTAAGAGCATGAGGCTGCAGCATCGCCACCTGCCTCAGTC
GTGCTTCATGTGCGTGAGCTGTGTGTGTGAACGTGTGTATCCTCAGTGTTTGATGCAATGGTGGCATA
GCTAAATATATAGTACAATTAGTATGCAATTAGTACATAATAAAATGATTGCATTATTTCAAGCCAAA
AGAGAAAAGCTATTTGGTTAAAGAAAAGCATTCTGAGTTAAAAGGAAAATAATACTTGGTATAGTTTC
AGGAAAAGTAGTTCTTAGAGATGAAAATGAATAAAGGTCACCAAGTATCCGAGAATAGGTAAATGAAA
CTCTGTCTTATTTTTAAGACCACATGCTGTTTGTGGTAAAACCTCAGTTTATGTGTCCCTGACATTGA
TTCTGCTTTTTAAAATTACAAAAGCAA

| | | | |
|---|---|---|---|
| NOV54d, CG59905-02 | SEQ ID NO:1116 | 810 aa | MW at 87208.0 kD |
| Protein Sequence | | | |

MCKPVACPSIEAQLSEHVTWRLVSGSLNEYGAQVLLSCSPGYYLEGWRLLRCQANGTWSIGDERPSCR
VISCGSLSFPPNGNKIGTLTVYGATAIFTCNTGYTLVGSHVRECLANGLWSGSETRCLAGHCGSPDPI
VNGHISGDGFSYRDTVVYQCNPGFRLVGTSVRICLQDHKWSGQTPVCVAITCGHPGNPAHGFTNGSEF
NLNDVVNFTCNTGYLLQGVSRAQCRSNGQWSSPLPTSRHVGRGLLHWRLLRHGAPRRALQQIARVAGE
IHDIIILLAGSSALTCMANGLWDRSLPKCLAISCGHPGVPANAVLTGELFTYGAVVHYSCRGSESLIG
NDTRVCQEDSHWSGALPHCTGNNPGFCGDPGTPAHGSRLGDDFKTKSLLRFSCEMGHQLRGSPERTCL
LNGSWSGLQSVCEAVSCGNPGTPTNGMIVSSDGILFSSSVIYACWEGYKTSGLMTRHCTANGTWTGTA
PDCLVISCGDPGTLANGIQFGTDFTFNKTVSYQCNPGYVMEAVTSATIRCTKDGRWNPSKPVCKGLLC
PQPPPVQNGTVEGSDFRWGSSISYSCMDGYQLSHSAILSCEGRGVWKGEIPQCLPVFCGDPGIPAEGR
LSGKSFTYKSEVFFQCKSPFILVGSSRRVCQADGTWSGIQPTCIDPAHNTCPDPGTPHFGIQNSSRGY
EVGSTVFFRCRKGYHIQGSTTRTCLANLTWSGIQTECIPHACRQPETPAHADVRAIDLPTFGYTLVYT
CHPGFFLAGGSEHRTCKADMKWTGKSPVCKSKSLIKEWHTFPKSKGKDQLPIRLVSTCETHK

| | | |
|---|---|---|
| NOV54e, SNP13382527 of | SEQ ID NO:1117 | 3145 bp |
| CG59905-01, DNA Sequence | ORF Start: ATG at 34 | ORF Stop: TGA at 3046 |
| | SNP Pos: 392 | SNP Change: T to C |

ATTTTACTTGCTGGATCTTCAGCCTTGACCTGTATGGCAAATGGCTTATGGGACCGATCCCTGCCCAA
GTGTTTGGCTATATCCTGTGGACACCCAGGGGTCCCTGCCAACGCCGTCCTCACTGGAGAGCTGTTTA
CCTATGGCGCCGTCGTGCACTACTCCTGCAGAGGGAGCGAGAGCCTCATAGGCAACGACACGAGAGTG
TGCCAGGAAGACAGTCACTGGAGCGGGGCACTGCCCCACTGCACAGGAAATAATCCTGGATTCTGTGG
TGATCCGGGGACCCCAGCACATGGGTCTCGGCTTGGTGATGACTTTAAGACAAAGAGTCTTCTCCGCT
TCTCCTGTGAAATGGGGCACCAGCTGAGGGGCTCCCCTGAACGCACGTGTTCGCTCAATGGGTCATGG
TCAGGACTGCAGCCGGTGTGTGAGGCCGTGTCCTGTGGCAACCCTGGCACACCCACCAACGGAATGAT
TGTCAGTAGTGATGGCATTCTGTTCTCCAGCTCGGTCATCTATGCCTGCTGGGAAGGCTACAAGACCT
CAGGGCTCATGACACGGCATTGCACAGCCAATGGGACCTGGACAGGCACTGCTCCCGACTGCACAATT
ATAAGTTGTGGGGATCCAGGCACACTAGCAAATGGCATCCAGTTTGGGACCGACTTCACCTTCAACAA
GACTGTGAGCTATCAGTGTAACCCAGGCTATGTCATGGAAGCAGTCACATCCGCCACTATTCGCTGTA
CCAAAGACGGCAGGTGGAATCCGAGCAAACCTGTCTGCAAAGCCGTGCTGTGTCCTCAGCCGCCGCCG
GTGCAGAATGGAACAGTGGAGGGAAGTGATTTCCGCTGGGGCTCCAGCATAAGTTACAGCTGCATGGA
CGGTTACCAGCTCTCTCACTCCGCCATCCTCTCCTGTGAAGGTCGCGGGGTGTGGAAAGGAGAGATCC
CCAGTGTCTGCCTGTGTTCTGCGGAGACCCTGGCATCCCCGCAGAAGGGCGACTTAGTGGGAAAAGTT
TCACCTATAAGTCCGAAGTCTTCTTCCAGTGCAAATCTCCATTTATACTCGTGGGATCCTCCAGAAGA
GTCTGCCAAGCTGACGGCACGTGGAGCGGCATACAACCCACCTGCATTGGTAATAATTATCATACAGC
TCTGGGGATACCTGGGAGTATTTGGAGATGAGGACGCTTCATTCCGAAATTGGGTCATTTGTGATTAC
ATAGAAAGTGTTTCCATAGACAGTTTTCTGTACAAGGTTGGAAGCACGGTTTTTTTCAGGTGCAGAAA
AGGCTACCATATTCAAGGTTCCACGACTCGCACCTGCCTTGCCAATTTAACATGGAGTGGGATACAGA
CCGAATGTATACCTCATGCCTGCAGACAGCCAGAAACCCCGGCACACGCGGATGTGAGAGCCATCGAT
CTTCCTACTTTCGGCTACACCTTAGTGTACACCTGCCATCCAGGCTTTTTCCTCGCAGGGGGATCTGA
GCACAGAACATGTAAAGCAGACATGAAATGGACAGGAAAGTCGCCTGTGTGTAAAATTCCTTCAGATG
TCTTTTTCGTCAATTCACTGTGGAAGGGGTATTATGAATATTTAGGGAAAAGACAACCCGCCACTCTA
ACTGTTGACTGGTTCAATGCAACAAGCAGTAAGGTGAATGCCACCTTCAGCGAAGCCTCGCCAGTGGA
GCTGAAGTTGACAGGCATTTACAAGAAGGAGGAGGCCCACTTACTCCTGAAAGCTTTTCAAATTAAAG
GCCAGGCAGATATTTTTGTAAGCAAGTTCGAAAATGACAACTGGGGACTAGATGGTTATGTGTCATCT
GGACTTGAAAGAGGAGGATTTACTTTTCAAGGTGACATTCATGGAAAAGACTTTGGAAAATTTAAGCT
AGAAAGGCAAGGATGGGTCACAATATTCTTGAGCCTATTTCTTCATCTTAAATCTCAGTATAGAAGTT
CCCAAGGTTGTTACGAGATTGAGAGGCCACATCCTTTAAACCCAGATCAAGACTCTTCCAGTCATTAC
CACGGCACCAGCAGTGGCTCTGTGGCGGCTGCCATTCTGGTTCCTTTCTTTGCTCTAATTTTATCAGG
GTTTGCATTTTACCTCTACAAACACAGAACGAGACCAAAAGTTCAATACAATGGCTATGCTGGGCATG
AAAACAGCAATGGACAAGCATCGTTTGAAAACCCCATGTATGATACAAACTTAAAACCCACAGAAGCC
AAGGCTAAAACCACACACGGGCTGCTCACGTCTCTGGACTCCAAGAGAAAGATGGTTATAACCCTGGC
AGAGTCTTTACCCGCCCTGCGAACCTGCCTGTTGATCTGCTTCCCACTCCTCTTTAGACTTGCTCGTG
AATCTTCAGCACAGCCATTTGTTTTGAGTATTCAAACACATAAAAAGATATCTGGCAACTTCCCACTT
CTTATGACTTCTCAGACACCTTGTAACGGAGCTTCCCTTGGAGGTGGACAAACTTCTCACTCACAAGA
GAGACGGAGAGCAACAGAGAGAGATGGGCAGGGATTGATTCGAGGATTGGCTGACTCGATTATGGAGG
GTGAGAAGTCCCAGGACAGGCCGTCTGCAAGGCGTAAACCCAGGGAAGCTGCTGGTATGGCTCGGTTC
AAGTCCAAAGGCCTCAGCACCAAGGAAACCAAGGTGATAACTCTCAGTTCGGGTCAAAGTCCTGGGAG
TCTGCAAGGCCTCTCATTGAGTAAGTCTCACAAGATCCGATGGTTTTATAAAGGGCAGTTCCCCTGCA
CAAGCTCTCTTCTCTGCCACCACGCGTTTGCTGTTCATTCACCTTTCACCATGATTGTGAGGCCTCCC
AGCCATGTGGAACTGTGCAAAGAGGATGTTCAAAGGGCATCTTGGACCAGGAGGAGGAAGCCCATTGT
GAGGATGGCAAAGAACAATAAATGGAAGGCCCGTGGAACCCTGAGCGGCTGCCCCATCACTGTCCTCT
CCATCCTTTCCATGCTTCCCATGGTGCATGGCTCTCAGGCTCTCCAAGCACCATGAGCTCCTGGCGCG
TATGGCCTGGCCCCTGAGTGTGGAGTCTGTGGACTTTAAATGTGCCTTTACTCAGCCAGCATCTCCCT
GGCTGGGTGCGATGTTG

| | | | |
|---|---|---|---|
| NOV54e, SNP13382527 of | SEQ ID NO:1118 | 1004 aa | MW at 109726.6 kD |
| CG59905-01, Protein | SNP Pos: 120 | | SNP Change: Leu to Ser |
| Sequence | | | |

MANGLWDRSLPKCLAISCGHPGVPANAVLTGELFTYGAVVHYSCRGSESLIGNDTRVCQEDSHWSGAL
PHCTGNNPGFCGDPGTPAHGSRLGDDFKTKSLLRFSCEMGHQLRGSPERTCSLNGSWSGLQPVCEAVS
CGNPGTPTNGMIVSSDGILFSSSVIYACWEGYKTSGLMTRHCTANGTWTGTAPDCTIISCGDPGTLAN
GIQFGTDFTFNKTVSYQCNPGYVMEAVTSATIRCTKDGRWNPSKPVCKAVLCPQPPPVQNGTVEGSDF
RWGSSISYSCMDGYQLSHSAILSCEGRGVWKGEIPSVCLCSAETLASPQKGDLVGKVSPISPKSSSSA
NLHLYSWDPPEESAKLTARGAAYNPPALVTIIIQLWGYLGVFGDEDASFRNWVICDYIESVSIDSFLY
KVGSTVFFRCRKGYHIQGSTTRTCLANLTWSGIQTECIPHACRQPETPAHADVRAIDLPTFGYTLVYT
CHPGFFLAGGSEHRTCKADMKWTGKSPVCKIPSDVFFVNSLWKGYYEYLGKRQPATLTVDWFNATSSK
VNATFSEASPVELKLTGIYKKEEAHLLLKAFQIKGQADIFVSKFENDNWGLDGYVSSGLERGGFTFQG
DIHGKDFGKFKLERQGWVTIFLSLFLHLKSQYRSSQGCYEIERPHPLNPDQDSSSHYHGTSSGSVAAA
ILVPFFALILSGFAFYLYKHRTRPKVQYNGYAGHENSNGQASFENPMYDTNLKPTEAKAKTTHGLLTS
LDSKRKMVITLAESLPALRTCLLICFPLLFRLARESSAQPFVLSIQTHKKISGNFPLLMTSQTPCNGA
SLGGGQTSHSQERRRATERDGQGLIRGLADSIMEGEKSQDRPSARRKPREAAGMARFKSKGLSTKETK
VITLSSGQSPGSLQGLSLSKSHKIRWFYKGQFPCTSSLLCHHAFAVHSPFTMIVRPPSHVELCKEDVQ
RASWTRRRKPIVRMAKNNKWKARGTLSGCPITVLSILSMLPMVHGSQALQAP

| | | |
|---|---|---|
| NOV54f, SNP13382520 of | SEQ ID NO:1119 | 3145 bp |
| CG59905-01, DNA Sequence | ORF Start: ATG at 34 | ORF Stop: TGA at 3046 |
| | SNP Pos: 1620 | SNP Change: A to T |

ATTTTACTTGCTGGATCTTCAGCCTTGACCTGTATGGCAAATGGCTTATGGGACCGATCCCTGCCCAA
GTGTTTGGCTATATCCTGTGGACACCCAGGGGTCCCTGCCAACGCCGTCCTCACTGGAGAGCTGTTTA
CCTATGGCGCCGTCGTGCACTACTCCTGCAGAGGGAGCGAGAGCCTCATAGGCAACGACACGAGAGTG
TGCCAGGAAGACAGTCACTGGAGCGGGGCACTGCCCCACTGCACAGGAAATAATCCTGGATTCTGTGG
TGCTCCGGGGACCCCAGCACATGGGTCTCGGCTTGGTGATGACTTTAAGACAAAGAGTCTTCTCCGCT
TCTCCTGTGAAATGGGGCACCAGCTGAGGGGCTCCCCTGAACGCACGTGTTTGCTCAATGGGTCATGG
TCAGGACTGCAGCCGGTGTGTGAGGCCGTGTCCTGTGGCAACCCTGGCACACCCACCAACGGAATGAT
TGTCAGTAGTGATGGCATTCTGTTCTCCAGCTCGGTCATCTATGCCTGCTGGGAAGGCTACAAGACCT
CAGGGCTCATGACACGGCATTGCACAGCCAATGGGACCTGGACAGGCACTGCTCCCGACTGCACAATT
ATAAGTTGTGGGGATCCAGGCACACTAGCAAATGGCATCCAGTTTGGGACCGACTTCACCTTCAACAA
GACTGTGAGCTATCAGTGTAACCCAGGCTATGTCATGGAAGCAGTCACATCCGCCACTATTCGCTGTA
CCAAAGACGGCAGGTGGAATCCGAGCAAACCTGTCTGCAAAGCCGTGCTGTGTCCTCAGCCGCCGCCG
GTGCAGAATGGAACAGTGGAGGGAAGTGATTTCCGCTGGGGCTCCAGCATAAGTTACAGCTGCATGGA
CGGTTACCAGCTCTCTCACTCCGCCATCCTCTCCTGTGAAGGTCGCGGGGTGTGGAAAGGAGAGATCC
CCAGTGTCTGCCTGTGTTCTGCGGAGACCCTGGCATCCCCGCAGAAGGGCGACTTAGTGGGAAAAGTT
TCACCTATAAGTCCGAAGTCTTCTTCCAGTGCAAATCTCCATTTATACTCGTGGGATCCTCCAGAAGA
GTCTGCCAAGCTGACGGCACGTGGAGCGGCATACAACCCACCTGCATTGGTAATAATTATCATACAGC
TCTGGGGATACCTGGGAGTATTTGGAGATGAGGACGCTTCATTCCGAAATTGGGTCATTTGTGATTAC
ATAGAAAGTGTTTCCATAGACAGTTTTCTGTACAAGGTTGGAAGCACGGTTTTTTTCAGGTGCAGAAA
AGGCTACCATATTCAAGGTTCCACGACTCGCACCTGCCTTGCCAATTTAACATGGAGTGGGATACAGA
CCGAATGTATACCTCATGCCTGCAGACAGCCAGAAACCCCGGCACACGCGGATGTGAGAGCCATCGAT
CTTCCTACTTTCGGCTACACCTTAGTGTACACCTGCCATCCAGGCTTTTTCCTCGCAGGGGGATCTGA
GCACAGAACATGTAAAGCAGACATGAAATGGACAGGAAAGTCGCCTGTGTGTAAAATTCCTTCAGATG
TCTTTTTCGTCAATTCACTGTGGAAGGGGTATTATGAATATTTAGGGAAAAGACATCCCGCCACTCTA
ACTGTTGACTGGTTCAATGCAACAAGCAGTAAGGTGAATGCCACCTTCAGCGAAGCCTCGCCAGTGGA
GCTGAAGTTGACAGGCATTTACAAGAAGGAGGAGGCCCACTTACTCCTGAAAGCTTTTCAAATTAAAG
GCCAGGCAGATATTTTTGTAAGCAAGTTCGAAAATGACAACTGGGGACTAGATGGTTATGTGTCATCT
GGACTTGAAAGAGGAGGATTTACTTTTCAAGGTGACATTCATGGAAAAGACTTTGGAAAATTTAAGCT
AGAAAGGCAAGGATGGGTCACAATATTCTTGAGCCTATTTCTTCATCTTAAATCTCAGTATAGAAGTT
CCCAAGGTTGTTACGAGATTGAGAGGCCACATCCTTTAAACCCAGATCAAGACTCTTCCAGTCATTAC
GTTTGCATTTTACCTCTACAAACACAGAACGAGACCAAAAGTTCAATACAATGGCTATGCTGGGCATG
AAAACAGCAATGGACAAGCATCGTTTGAAAACCCCATGTATGATACAAACTTAAAACCCACAGAAGCC
AAGGCTAAAACCACACACGGGCTGCTCACGTCTCTGGACTCCAAGAGAAAGATGGTTATAACCCTGGC
AGAGTCTTTACCCGCCCTGCGAACCTGCCTGTTGATCTGCTTCCCACTCCTCTTTAGACTTGCTCGTG
AATCTTCAGCACAGCCATTTGTTTTGAGTATTCAAACACATAAAAAGATATCTGGCAACTTCCCACTT
CTTATGACTTCTCAGACACCTTGTAACGGAGCTTCCCTTGGAGGTGGACAAACTTCTCACTCACAAGA
GAGACGGAGAGCAACAGAGAGAGATGGGCAGGGATTGATTCGAGGATTGGCTGACTCGATTATGGAGG
GTGAGAAGTCCCAGGACAGGCCGTCTGCAAGGCGTAAACCCAGGGAAGCTGCTGGTATGGCTCGGTTC
AAGTCCAAAGGCCTCAGCACCAAGGAAACCAAGGTGATAACTCTCAGTTCGGGTCAAAGTCCTGGGAG
TCTGCAAGGCCTCTCATTGAGTAAGTCTCACAAGATCCGATGGTTTTATAAAGGGCAGTTCCCCTGCA
CAAGCTCTCTTCTCTGCCACCACGCGTTTGCTGTTCATTCACCTTTCACCATGATTGTGAGGCCTCCC
AGCCATGTGGAACTGTGCAAAGAGGATGTTCAAAGGGCATCTTGGACCAGGAGGAGGAAGCCCATTGT
GAGGATGGCAAAGAACAATAAATGGAAGGCCCGTGGAACCCTGAGCGGCTGCCCCATCACTGTCCTCT
CCATCCTTTCCATGCTTCCCATGGTGCATGGCTCTCAGGCTCTCCAAGCACCATGAGCTCCTGGCGCG
TATGGCCTGGCCCCTGAGTGTGGAGTCTGTGGACTTTAAATGTGCCTTTACTCAGCCAGCATCTCCCT
GGCTGGGTGCGATGTTG

| | | | |
|---|---|---|---|
| NOV54f, SNP13382520 of | SEQ ID NO:1120 | 1004 aa | MW at 109761.8 kD |
| CG59905-01, Protein Sequence | SNP Pos: 529 | | SNP Change: Gln to His |

MANGLWDRSLPKCLAISCGHPGVPANAVLTGELFTYGAVVHYSCRGSESLIGNDTRVCQEDSHWSGAL
PHCTGNNPGFCGDPGTPAHGSRLGDDFKTKSLLRFSCEMGHQLRGSPERTCLLNGSWSGLQPVCEAVS
CGNPGTPTNGMIVSSDGILFSSSVIYACWEGYKTSGLMTRHCTANGTWTGTAPDCTIISCGDPGTLAN
GIQFGTDFTFNKTVSYQCNPGYVMEAVTSATIRCTKDGRWNPSKPVCKAVLCPQPPPVQNGTVEGSDF
RWGSSISYSCMDGYQLSHSAILSCEGRGVWKGEIPSVCLCSAETLASPQKGDLVGKVSPISPKSSSSA
NLHLYSWDPPEESAKLTARGAAYNPPALVIIIIQLWGYLGVFGDEDASFRNWVICDYIESVSIDSFLY
KVGSTVFFRCRKGYHIQGSTTRTCLANLTWSGIQTECIPHACRQPETPAHADVRAIDLPTFGYTLVYT
CHPGFFLAGGSEHRTCKADMKWTGKSPVCKIPSDVFFVNSLWKGYYEYLGKRHPATLTVDWFNATSSK
VNATFSEASPVELKLTGIYKKEEAHLLLKAFQIKGQADIFVSKFENDNWGLDGYVSSGLERGGFTFQG
DIHGKDFGKFKLERQGWVTIFLSLFLHLKSQYRSSQGCYEIERPHPLNPDQDSSSHYHGTSSGSVAAA
ILVPFFALILSGFAFYLYKHRTRPKVQYNGYAGHENSNGQASFENPMYDTNLKPTEAKAKTTHGLLTS
LDSKRKMVITLAESLPALRTCLLICFPLLFRLARESSAQPFVLSIQTHKKISGNFPLLMTSQTPCNGA
SLGGGQTSHSQERRRATERDGQGLIRGLADSIMEGEKSQDRPSARRKPREAAGMARFKSKGLSTKETK
VITLSSGQSPGSLQGLSLSKSHKIRWFYKGQFPCTSSLLCHHAFAVHSPFTMIVRPPSNVELCKEDVQ
RASWTRRRKPIVRMAKNNKWKARGTLSGCPITVLSILSMLPMVHGSQALQAP

A ClustalW comparison of the above protein sequences yields the following sequence alignment shown in Table 54B.

TABLE 54B
Comparison of the NOV54 protein sequences.

NOV54a ------------------------------------------------------------
NOV54b MTAWRRFQSLLLLLGLLVLCARLLTAAKGQNCGGLVQGPNGTIESPGFPHGYPNYANCTW
NOV54c ------------------------------------------------------------
NOV54d ------------------------------------------------------------
NOV54a ------------------------------------------------------------
NOV54b IIITGERNRIQLSFHTFALEENFDILSVYDGQPQQGNLKVRLSGFQLPSSIVSTGSILTL
NOV54c ------------------------------------------------------------
NOV54d ------------------------------------------------------------
NOV54a ------------------------------------------------------------
NOV54b WFTTDFAVSAQGFKALYEVLPSHTCGNPGEILKGVLHGTRFNIGDKIRYSCLPGYILEGH
NOV54c ------------------------------------------------------------
NOV54d ------------------------------------------------------------
NOV54a ------------------------------------------------------------
NOV54b AILTCIVSPGNGASWDFPAPFCHAEGACGGTLRGTSSSISSPHFPSEYENNADCTWTILA
NOV54c ------------------------------------------------------------
NOV54d ------------------------------------------------------------
NOV54a ------------------------------------------------------------
NOV54b EPGDTIALVFTDFQLEEGYDFLEISGTEAPSIWLTGMNLPSPVISSKNWLRLHFTSDSNH
NOV54c ------------------------------------------------------------
NOV54d ------------------------------------------------------------
NOV54a ------------------------------------------------------------
NOV54b RRKGFNAQFQVKKAIELKSRGVKMLPSKDGSHKNSVLSQGGVALVSHMCLDPGIPENGRR
NOV54c ------------------------------------------------------------
NOV54d ------------------------------------------------------------
NOV54a ------------------------------------------------------------
NOV54b AGSDFSRVGANVQFSCEDNYVLQGSKSTTCQRVTETLAAWSDHRPICRARTCGSNLRGPS
NOV54c ------------------------------------------------------------
NOV54d ------------------------------------------------------------
NOV54a ------------------------------------------------------------
NOV54b GVITSPNYPVQYEDNAHCVWVITTTDPDKVIKLAFEEFELERGYDTLTVGDAGKVGDTRS
NOV54c ------------------------------------------------------------
NOV54d ------------------------------------------------------------
NOV54a ------------------------------------------------------------
NOV54b VLYVLTGSSVPDLIVSMSNQMWLHLQSDDSIGSPGFKAVYQEIEKGGCGDPGIPAYGKRT
NOV54c ------------------------------------------------------------
NOV54d ------------------------------------------------------------
NOV54a ------------------------------------------------------------
NOV54b GSSFLHGDTLTFECPAAFELVGERVITCQQNNQWSGNKPSCVFSCFFNFTASSGIILSPN
NOV54c ------------------------------------------------------------
NOV54d ------------------------------------------------------------
NOV54a ------------------------------------------------------------
NOV54b YPEEYGNNMNCVWLTISEPGSRIHLIFNDFDVEPQFDFLAVKDNGISDITVLGTFSGNEV
NOV54c ------------------------------------------------------------
NOV54d ------------------------------------------------------------
NOV54a ------------------------------------------------------------
NOV54b PSQLASSGHIVRLEFQSDHSTTGRGFNITYTTFGQNECHDPGIPINGRRFGDRFLLGSSV
NOV54c ------------------------------------------------------------
NOV54d ------------------------------------------------------------
NOV54a ------------------------------------------------------------
NOV54b SFHCDDGFVKTQGSESITCILQDGNVVWSSTVPRCEAPCGGHLTASSGVILPPGWPGYYK
NOV54c ------------------------------------------------------------
NOV54d ------------------------------------------------------------
NOV54a ------------------------------------------------------------
NOV54b DSLHCEWIIEAKPGHSIKMTFDRFQTEVNYDTLEVRDGPASSSPLIGEYHGTQAPQFLIS
NOV54c ------------------------------------------------------------
NOV54d ------------------------------------------------------------
NOV54a ------------------------------------------------------------
NOV54b TGNFMYLLFTTDNSRSSIGFLIHYESVTLESDSCLDPGIPVNGHRHGGDFGIRSTVTFSC
NOV54c ------------------------------------------------------------
NOV54d ------------------------------------------------------------
NOV54a ------------------------------------------------------------
NOV54b DPGYTLSDDEPLVCERNHQWNHALPSCDALCGGYIQGKSGTVLSPGFPDFYPNSLNCTWT
NOV54c ------------------------------------------------------------
NOV54d ------------------------------------------------------------
NOV54a ------------------------------------------------------------
NOV54b IEVSHGKGVQMIFHTFHLESSHDYLLITEDGSFSEPVARLTGSVLPHTIKAGLFGNFTAQ
NOV54c ------------------------------------------------------------
NOV54d ------------------------------------------------------------
NOV54a ------------------------------------------------------------
NOV54b LRFISDFSISYEGFNITFSEYDLEPCDDPGVPAFSRRIGFHFGVGDSLTFSCFLGYRLEG
NOV54c ------------------------------------------------------------
NOV54d ------------------------------------------------------------
NOV54a ------------------------------------------------------------
NOV54b ATKLTCLGGGRRVWSAPLPRCVAECGASVKGNEGTLLSPNFPSNYDNTHECIYKIETEAG
NOV54c ------------------------------------------------------------
NOV54d ------------------------------------------------------------
NOV54a ------------------------------------------------------------
NOV54b KGIHLRTRSFQLFEGDTLKVYDGKDSSSRPLGTFTKNELLGLILNSTSNHLWLEFNTNGS
NOV54c ------------------------------------------------------------
NOV54d ------------------------------------------------------------
NOV54a ------------------------------------------------------------
NOV54b DTDQGFQLTYTSFDLVKCEDPGIPNYGYRIRDEGHFTDTVVLYSCNPGYANHGSNTLTCL
NOV54c ------------------------------------------------------------
NOV54d ------------------------------------------------------------
NOV54a ------------------------------------------------------------
NOV54b SGDPRVWDKPLPSCIAECGGQIHAATSGRILSPGYPAPYDNNLHCTWIIEADPGKTISLH
NOV54c ------------------------------------------------------------
NOV54d ------------------------------------------------------------
NOV54a ------------------------------------------------------------
NOV54b FIVFDTEMAHDILKVWDGPVDSDILLKEWSGSALPEDIHSTFNSLTLQFDSDFFISKSGF
NOV54c ------------------------------------------------------------
NOV54d ------------------------------------------------------------
NOV54a ------------------------------------------------------------
NOV54b SIQFSTSIAATCNDPGMPQNGTRYGDSREAGDTVTFQCDPGYQLQGQAKITCVQLNNRFF
NOV54c ------------------------------------------------------------
NOV54d ------------------------------------------------------------
NOV54a ------------------------------------------------------------
NOV54b WQPDPPTCIAACGGNLTGPAGVILSPNYPQPYPPGKECDWRVKVNPDFVIALIFKSFNME
NOV54c ------------------------------------------------------------
NOV54d ------------------------------------------------------------
NOV54a ------------------------------------------------------------
NOV54b PSYDFLHIYEGEDSNSPLIGSYQGSQAPERIESSGNSLFLAFRSDASVGLSGFAIEFKEK
NOV54c ------------------------------------------------------------
NOV54d ------------------------------------------------------------
NOV54a ------------------------------------------------------------
NOV54b PREACFDPGNIMNGTRVGTDFKLGSTITYQCDSGYKILDPSSITCVIGADGKPSWDQVLP
NOV54c ------------------------------------------------------------
NOV54d ------------------------------------------------------------
NOV54a ------------------------------------------------------------
NOV54b SCNAPCGGQYTGSEGVVLSPNYPHNYTAGQICLYSITVPKEFVVFGQFAYFQTALNDLAE
NOV54c ------------------------------------------------------------
NOV54d ------------------------------------------------------------
NOV54a ------------------------------------------------------------
NOV54b LFDGTHAQARLLSSLSGSHSGETLPLATSNQILLRFSAKSGASARGFHFVYQAVPRTSDT
NOV54c ------------------------------------------------------------
NOV54d ------------------------------------------------------------
NOV54a ------------------------------------------------------------
NOV54b QCSSVPEPRYGRRIGSEFSAGSIVRFECNPGYLLQGSTALHCQSVPNALAQWNDTIPSCV
NOV54c ------------------------------------------------------------
NOV54d ------------------------------------------------------------
NOV54a ------------------------------------------------------------
NOV54b VPCSGNFTQRRGTILSPGYPEPYGNNLNCIWKIIVTEGSGIQDPSDQFCHGAELGLPFEI
NOV54c ------------------------------------------------------------
NOV54d ------------------------------------------------------------
NOV54a ------------------------------------------------------------
NOV54b HDGGDVTAPRLGSFSGTTVPALLNSTSNQLYLHFQSDISVAAAGFHLEYKTVGLAACQEP
NOV54c ------------------------------------------------------------
NOV54d ------------------------------------------------------------
NOV54a ------------------------------------------------------------
NOV54b ALPSNSIKIGDRYMVNDVLSFQCEPGYTLQGRSHISCMPGTVRRWNYPSPLCIATCGGTL
NOV54c ------------------------------------------------------------
NOV54d ------------------------------------------------------------
NOV54a ------------------------------------------------------------
NOV54b STLGGVILSPGFPGSYPNNLDCTWRISLPIGYGAHIQFLNFSTEANHDFLEIQNGPYHTS
NOV54c ------------------------------------------------------------
NOV54d ------------------------------------------------------------
NOV54a ------------------------------------------------------------
NOV54b PMIGQFSGTDLPAALLSTTHETLIHFYSDHSQNRQGFKLAYQAYELQNCPDPPPFQNGYM
NOV54c ------------------------------------------------------------
NOV54d ------------------------------------------------------------
NOV54a ------------------------------------------------------------
NOV54b INSDYSVGQSVSFECYPGYILIGHPVLTCQHGINRNWNYPFPRCDAPCGYNVTSQNGTIY
NOV54c ------------------------------------------------------------
NOV54d ------------------------------------------------------------
NOV54a ------------------------------------------------------------
NOV54b SPGFPDEYPILKDCIWLITVPPGHGVYINFTLLQTEAVNDYIAVWDGPDQNSPQLGVFSG
NOV54c ------------------------------------------------------------
NOV54d ------------------------------------------------------------
NOV54a ------------------------------------------------------------
NOV54b NTALETAYSSTNQVLLKFHSDFSNGGFFVLNFHAFQLKKCQPPPAVPQAEMLTEDDDFEI
NOV54c ------------------------------------------------------------
NOV54d ------------------------------------------------------------
NOV54a ------------------------------------------------------------
NOV54b GDFVKYQCHPGYTLVGTDILTCKLSSQLQFEGSLPTCEAQCPANEVRTGSSGVILSPGYP
NOV54c ------------------------------------------------------------
NOV54d ------------------------------------------------------------
NOV54a ------------------------------------------------------------
NOV54b GNYFNSQTCSWSIKVEPNYNITIFVDTFQSEKQFDALEVFDGSSGQSPLLVVLSGNHTEQ
NOV54c ------------------------------------------------------------
NOV54d ------------------------------------------------------------
NOV54a ------------------------------------------------------------
NOV54b SNFTSRSNQLYLRWSTDHATSKKGFKIRYAAPYCSLTHPLKNGGILNRTAGAVGSKVHYF
NOV54c ------------------------------------------------------------
NOV54d ------------------------------------------------------------
NOV54a ------------------------------------------------------------
NOV54b CKPGYRMVGHSNATCRRNPLGMYQWDSLTPLCQAVSCGIPESPGNGSFTGNEFTLDSKVV
NOV54c ------------------------------------------------------------
NOV54d ------------------------------------------------------------
NOV54a ------------------------------------------------------------
NOV54b YECHEGFKLESSQQATAVCQEDGLWSNKGKPPMCKPVACPSIEAQLSEHVIWRLVSGSLN
NOV54c ------------------------------------------------------------
NOV54d --------------------------------MCKPVACPSIEAQLSENVIWRLVSGSLN
NOV54a ------------------------------------------------------------
NOV54b EYGAQVLLSCSPGYYLEGWRLLRCQANGTWNIGDERPSCRVISCGSLSFPPNGNKIGTLT
NOV54c ------------------------------------------------------------
NOV54d EYGAQVLLSCSPGYYLEGWRLLRCQANGTWSIGDERPSCRVISCGSLSFPPNGNKIGTLT
NOV54a ------------------------------------------------------------
NOV54b VYGATAIFTCNTGYTLVGSHVRECLANGLWSGSETRCLAGHCGSPDPIVNGHISGDGFSY
NOV54c ------------------------------------------------------------
NOV54d VYGATAIFTCNTGYTLVGSHVRECLANGLWSGSETRCLAGHCGSPDPIVNGHISGDGFSY
NOV54a ------------------------------------------------------------
NOV54b RDTVVYQCNPGFRLVGTSVRICLQDHKWSGQTPVCVRITCGHPGNPAHGFTNGSEFNLND
NOV54c ------------------------------------------------------------
NOV54d RDTVVYQCNPGFRLVGTSVRICLQDHKWSGQTPVCVAITCGHPGNPAHGFTNGSEFNLND
NOV54a ------------------------------------------------------------
NOV54b VVNFTCNTGYLLQGVSPAQCRSNGQWSSPLPTCRVVNCSDPGFVENAIRHGQQNFPESFE
NOV54c ------------------------------------------------------------
NOV54d VVNFTCNTGYLLQGVSRAQCRSNGQWSSPLPTSRHVGRG--LLHWRLLRHG-APRHALQQ
NOV54a -----------------------MANGLWDRSLPKCLATSCGHPGVPANAVLTGELFTYG
NOV54b YGMSILYHCKKGFYLLGSSALTCMANGLWDRSLPKCLAISCGHPGVPANAVLTGELFTYG
NOV54c -------------------------------------TRSCGHPGVPANAVLTGELFTYG
NOV54d IARVAGEIHDIIILLAGSSALTCMANGLWDRSLPKCLAISCGHPGVPANAVLTGELFTYG
NOV54a AVVHYSCRGSESLIGNDTRVCQEDSHWSGALPHCTGNNPGFCGDPGTPAHGSRLGDDFKT
NOV54b AVVHYSCRGSESLIGNDTRVCQEDSHWSGALPHCTGNNPGFCGDPGTPAHGSRLGDDFKT
NOV54c AVVHYSCRGSESLIGNDTRVCQEDSHWSGALPHCTGNNPGFCGDPGTPAHGSRLGDDFKT
NOV54d AVVHYSCRGSESLIGNDTRVCQEDSHWSGALPHCTGNNPGFCGDPGTPAHGSRLGDDFKT
NOV54a KSLLRFSCEMGHQLRGSPERTCLLNGSWSGLQPVCEAVSCGNPGTPTNGMIVSSDGILFS
NOV54b KSLLRFSCEMGHQLRGSPERTCLLNGSWSGLQPVCEAVSCGNPGTPTNGMIVSSDGILFS
NOV54c KSLLRFSCEMGHQLRGSPERTCLLNGSWSGLQPVCEAVSCGNPGTPTNGMIVSSDGILFS
NOV54d KSLLRFSCEMGHQLRGSPERTCLLNGSWSGLQSVCEAVSCGNPGTPTNGMIVSSDGILFS
NOV54a SSVIYACWEGYKTSGLMTRHCTANGTWTGTAPDCTIISCGDPGTLANGIQFGTDFTFNKT
NOV54b SSVIYACWEGYKTSGLMTRHCTANGTWTGTAPDCTVISCGDPGTLANGIQFGTDFTFNKT
NOV54c SSVIYACWEGYKTSGLMTRHCTANGTWTGTAPDCTIISCGDPGTLANGIQFGTDFTFNKT
NOV54d SSVIYACWEGYKTSGLMTRHCTANGTWTGTAPDCLVISCGDPGTLANGIQFGTDFTFNKT
NOV54a VSYQCNPGYVMEAVTSATIRCTKDGRWNPSKPVCKAVLCPQPPPVQNGTVEGSDFRWGSS
NOV54b VSYQCNPGYVMEAVTSATIRCTKDGRWNPSKPVCKAVLCPQPPPVQNGTVEGSDFRWGSS
NOV54c VSYQCNPGYVMEAVTSATIRCTKDGRWNPSKPVCKAVLCPQPPPVQNGTVEGSDFRWGSS
NOV54d VSYQCNPGYVMEAVTSATIRCTKDGRWNPSKPVCKGLLCPQPPPVQNGTVEGSDFRWGSS
NOV54a ISYSCMDGYQLSHSAILSCEGRGVWKGEIPSVCLCSAETIASPQKGDLVGKVSPISPKSS
NOV54b ISYSCMDGYQLSHSAILSCEGRGVWKGEIPQCLAVFCGDPGIPAEGRLSGKSFTYKSEVF
NOV54c ISYSCMDGYQLSHSAILSCEGRGVWKGEIPSVCLEG------------------------
NOV54d ISYSCMDGYQLSHSAILSCEGRGVWKGEIPQCLPVFCGDPGIPAEGRLSGKSFTYKSEVF
NOV54a SSANLHLYSWDPPEESAKLTARGAAYNPPALVIIIIQLWGYLGVFGDEDASFRNWVICDY
NOV54b FQCKSPFILVGSSRRVCQADGTWSGIQPTCIGDPAHNTCPDPGTPHFG------------
NOV54c ------------------------------------------------------------
NOV54d FQCKSPFILVGSSRRVCQADGTWSGIQPTCI-DPAHNTCPDPGTPHFG------------
NOV54a IESVSIDSFLYKVGSTVFFRCRKGYHIQGSTTRTCLANLTWSGIQTECIPHACRQPETPA
IWV54b ---IQNSSRGYEVGSTVFFRCRKGYHIQGSTTRTCLANLTWSGIQTECIRHACRQPETPA
NOV54c ------------------------------------------------------------
NOV54d ---IQNSSRGYEVGSTVFFRCRKGYHIQGSTTRTCLANLTWSGIQTECIPHACRQPETPA
NOV54a HADVRAIDLPTFGYTLVYTCHPGFFLAGGSEHRTCKADMKWTGKSPVCKIP---------
NOV54b HADVRAIDLPTFGYTLVYTCHPGFFLAGGSEHRTCKADMKWTGKSPVCKSKGVREVNETV
NOV54c ------------------------------------------------------------
NOV54d HADVRAIDLPTFGYTLVYTCHPGFFLAGGSEHRTCKADMKWTGKSPVCKSK---------
NOV54a ------SDVFFVNSLWKGYYEYLGKRQPATLTVDWFNATSSKVNATFSEASPVELKLTGI
NOV54b TKTPVPSDVFFVNSLWKGYYEYLGKRQPATLTVDWFNATSSKVNATFSEASPVELKLTGI
NOV54c ------------------------------------------------------------
NOV54d -------------SLIKEWHTFPKSKGKDQLPIRLVSTCETHK-----------------
NOV54a YKKEEAHLLLKAFQIKGQADIFVSKFENDNWGLDGYVSSGLERGGFTFQGDIHGKDFGKF
NOV54b YKKEEAHLLLKAFQIKGQADIFVSKFENDNWGLDGYVSSGLERGGFTFQGDIHGKDFGKF
NOV54c ------------------------------------------------------------
NOV54d ------------------------------------------------------------
NOV54a KLERQGWVTIFLSLFLHLKSQYRSSQGCYEIERPHPLNPDQDSSSHYHGTSSGSVAAAIL
NOV54b KLERQ-----------------------------DPLNPDQDSSSHYHGTSSGSVAAAIL
NOV54c ------------------------------------------------------------
NOV54d ------------------------------------------------------------
NOV54a VPFFALILSGFAFYLYKHRTRPKVQYNGYAGHENSNGQASFENPMYDTNLKPTEAKAXTT
NOV54b VPFFALILSGFAFYLYKHRTRPKVQYNGYAGHENSNGQASFENPMYDTNLKPTEAKAVRF
NOV54c ------------------------------------------------------------
NOV54d ------------------------------------------------------------
NOV54a HGLLTSLDSKRKMVITLAESLPALRTCLLICFPLLFRLARESSAQPFVLSIQTHKKISGN
NOV54b DTTLNTVCTVV-------------------------------------------------
NOV54c ------------------------------------------------------------
NOV54d ------------------------------------------------------------
NOV54a FPLLMTSQTPCNGASLGGGQTSHSQERPPATERDGQGLIRGLADSIMEGEKSQDRPSARR
NOV54b ------------------------------------------------------------
NOV54c ------------------------------------------------------------
NOV54d ------------------------------------------------------------
NOV54a KPREAAGMARFKSKGLSTKETKVITLSSGQSPGSLQGLSLSKSHKIRWFYKGQFPCTSSL
NOV54b ------------------------------------------------------------
NOV54c ------------------------------------------------------------
NOV54d ------------------------------------------------------------
NOV54a LCHHAFAVHSPFTMIVRPPSHVELCKEDVQRASWTRPRKPIVRMAKNNKWKARGTLSGCP
NOV54b ------------------------------------------------------------
NOV54c ------------------------------------------------------------
NOV54d ------------------------------------------------------------
NOV54a ITVLSILSMLPMVHGSQALQAP
NOV54b ----------------------
NOV54c ----------------------
NOV54d ----------------------
NOV54a (SEQ ID NO: 1110)
NOV54b (SEQ ID NO: 1112)
NOV54c (SEQ ID NO: 1114)
NOV54d (SEQ ID NO: 1116)

Further analysis of the NOV54a protein yielded the following properties shown in Table 54C.

TABLE 54C
Protein Sequence Properties NOV54a

| | |
|---|---|
| SignalP analysis: | No Known Signal Sequence Predicted |
| PSORT II analysis: | |

| PSG: | a new signal peptide prediction method | |
|---|---|---|
| | N-region: | length 8; pos. chg 1; neg. chg 1 |
| | H-region: | length 3; peak value 2.05 |
| | PSG score: | −2.35 |

| | |
|---|---|
| GvH: | von Heijne's method for signal seq. recognition |
| | GvH score (threshold: −2.1): −6.14 |
| | possible cleavage site: between 46 and 47 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 4 | | |
| | INTEGRAL | Likelihood = | Transmembrane 367-383 |
| | | −4.57 | |
| | INTEGRAL | Likelihood = | Transmembrane 677-693 |
| | | −7.80 | |
| | INTEGRAL | Likelihood = | Transmembrane 762-778 |
| | | −1.91 | |
| | INTEGRAL | Likelihood = | Transmembrane 979-995 |
| | | −0.80 | |

| | |
|---|---|
| PERIPHERAL | Likelihood = 1.75 (at 148) |

| | |
|---|---|
| | ALOM score: −7.80 (number of TMSs: 4) |
| MTOP: | Prediction of membrane topology (Hartmann et al.) |
| | Center position for calculation: 374 |
| | Charge difference: −2.0 C(−3.0)-N(−1.0) |
| | N >= C: N-terminal side will be inside |

>>> membrane topology: type 3a

| MITDISC: | discrimination of mitochondrial targeting seq | | | |
|---|---|---|---|---|
| | R content: | 1 | Hyd Moment(75): | 14.35 |
| | Hyd Moment(95): | 11.39 | G content: | 4 |
| | D/E content: | 2 | S/T content: | 3 |

| | |
|---|---|
| | Score: −5.37 |
| Gavel: | prediction of cleavage sites for mitochondrial preseq |
| | R-2 motif at 55 CRG\|SE |

NUCDISC: discrimination of nuclear localization signals

| | |
|---|---|
| | pat4: RRKP (4) at 861 |
| | pat4: RKPR (4) at 862 |
| | pat4: RRRK (5) at 958 |
| | pat4: RRKP (4) at 959 |
| | pat7: PSARRKP (4) at 858 |
| | bipartite: RRKPIVRMAKNNKWKAR at 959 |
| | content of basic residues: 10.4% |
| | NLS Score: 1.43 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs:
Ribosomal protein L23 signature (PS00050): *** found ***
RKMVITLAESLPALRT at 753
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 70.6 |
| COIL: | Lupas's algorithm to detect coiled-coil regions |
| | total: 0 residues |

Final Results (k = 9/23):

```
34.8%: nuclear
30.4%: mitochondrial
26.1%: endoplasmic reticulum
  4.3%: cytoplasmic
  4.3%: peroxisomal
```

>> prediction for CG59905-01 is nuc (k = 23)

A search of the NOV54a protein against the Geneseq database, a proprietary database that contains sequences published in patents and patent publication, yielded several homologous proteins shown in Table 54D.

TABLE 54D
Geneseq Results for NOV54a

| | | NOV54a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE20901 | Human C3b/C4b complement receptor | 1 . . . 328 | 312/328 (95%) | 0.0 |
| | like protein #2, alternative version - | 2378 . . . 2705 | 313/328 (95%) | |
| | Homo sapiens, 3100 aa. | | | |
| | [WO200210199-A2, 07-FEB-2002] | | | |
| AAE20900 | Human C3b/C4b complement receptor | 1 . . . 328 | 312/328 (95%) | 0.0 |
| | like protein #1 , alternative version - | 2347 . . . 2674 | 313/328 (95%) | |
| | Homo sapiens, 3069 aa. | | | |
| | [WO200210199-A2, 07-FEB-2002] | | | |
| AAE20789 | Human C3b/C4b complement receptor | 1 . . . 328 | 312/328 (95%) | 0.0 |
| | like protein #2 - Homo sapiens, 3100 | 2378 . . . 2705 | 313/328 (95%) | |
| | aa. [WO200210199-A2, 07-FEB-2002] | | | |
| AAE20787 | Human C3b/C4b complement receptor | 1 . . . 328 | 312/328 (95%) | 0.0 |
| | like protein #1 - Homo sapiens, 3069 | 2347 . . . 2674 | 313/328 (95%) | |
| | aa. [WO200210199-A2, 07-FEB-2002] | | | |
| ABU11478 | Human MDDT polypeptide SEQ ID 425 - | 27 . . . 328 | 286/302 (94%) | e−176 |
| | Homo sapiens, 474 aa. | 44 . . . 345 | 287/302 (94%) | |
| | [WO200279449-A2, 10-OCT-2002] | | | |

In a BLAST search of public sequence databases, the NOV54a protein was found to have homology to the proteins shown in the BLASTP data in Table 54E.

TABLE 54E
Public BLASTP Results for NOV54a

| | | NOV54a | Identities/ | |
|---|---|---|---|---|
| Protein | | Residues/ | Similarities for | |
| Accession | | Match | the Matched | Expect |
| Number | Protein/Organism/Length | Residues | Portion | Value |
| AAK73475 | CUB and sushi multiple domains 1 | 1 . . . 328 | 312/328 (95%) | 0.0 |
| | protein - Homo sapiens (Human), | 2844 . . . 3171 | 313/328 (95%) | |
| | 3566 aa. | | | |
| CAD26764 | Sequence 6 from Patent | 1 . . . 328 | 312/328 (95%) | 0.0 |
| | WO0210199 - Homo sapiens | 2378 . . . 2705 | 313/328 (95%) | |
| | (Human), 3100 aa (fragment). | | | |
| CAD26763 | Sequence 1 from Patent | 1 . . . 328 | 312/328 (95%) | 0.0 |
| | WO0210199 - Homo sapiens | 2347 . . . 2674 | 313/328 (95%) | |
| | (Human), 3069 aa. | | | |
| Q96RM4 | CUB and sushi multiple domains 1 | 1 . . . 328 | 312/328 (95%) | 0.0 |
| | protein - Homo sapiens (Human), | 2786 . . . 3113 | 313/328 (95%) | |
| | 3508 aa. | | | |
| Q923L3 | CSMD1 - Mus musculus (Mouse), | 1 . . . 328 | 279/328 (85%) | e−175 |
| | 3564 aa. | 2842 . . . 3169 | 297/328 (90%) | |

PFam analysis predicts that the NOV54a protein contains the domains shown in the Table 54F.

TABLE 54F
Domain Analysis of NOV54a

| | | Identities/ | |
|---|---|---|---|
| | | Similarities | |
| Pfam | NOV54a | for the | |
| Domain | Match Region | Matched Region | Expect Value |
| sushi | 18 . . . 71 | 18/63 (29%) | 2.9e−11 |
| | | 43/63 (68%) | |
| sushi | 79 . . . 132 | 15/63 (24%) | 4e−11 |
| | | 41/63 (65%) | |
| sushi | 137 . . . 191 | 20/64 (31%) | 1.4e−08 |
| | | 41/64 (64%) | |
| sushi | 196 . . . 251 | 21/65 (32%) | 5.2e−14 |
| | | 46/65 (71%) | |
| sushi | 256 . . . 310 | 20/64 (31%) | 4e−09 |
| | | 45/64 (70%) | |
| sushi | 395 . . . 445 | 18/62 (29%) | 3.2e−07 |
| | | 39/62 (63%) | |
| sushi | 450 . . . 505 | 18/64 (28%) | 1.4e−09 |
| | | 41/64 (64%) | |

### Example 55

The NOV55 clone was analyzed, and the nucleotide and encoded polypeptide sequences are shown in Table 55A.

TABLE 55A
NOV55 Sequence Analysis

| | | |
|---|---|---|
| NOV55a, CG59932-01 | SEQ ID NO: 1121 | 1171 bp |
| DNA Sequence | ORF Start: at 2 | ORF Stop: TAA at 1031 |

GGAGGCCGCAAGGCCCTTCGCCCGGGAGTGGAGGGCCCAGTCCCTACCCCTGGCAGTAGGGGGCGTTT
TGAAGCTGCGGCTCTGTGAGCTGTGGCTACTGCTACTGGGTTCTAGTTTGAACGCCAGATTTTTGCCA
GACGAGGAGGACGTAGACTTTATCAACGAGTACGTGAACCTCCACAATGAGCTGCGGGGCGACGTTAT
TCCCCGAGGGTCTAACTTGCGCTTCATGACTTGGGATGTAGCTTTATCACGGACTGCTAGAGCATGGG
GAAAAAAATGTTTGTTTACGCATAATATTTATTTACAAGATGTACAAATGGTCCATCCTAAATTTTAT
GGTATTGGTGAAAATATGTGGGTCGGCCCTGAAAATGAATTTACTGCAAGTATTGCTATCAGAAGTTG
GCATGCAGAGAAGAAAATGTACAATTTTGAAAATGGCAGTTGCTCTGGAGACTGTTCTAATTATATTC
AGCTTGTTTGGGACCACTCTTACAAAGTTGGTTGTGCTGTTACTCCATGTTCAAAAATTGGACATATT
ATACATGCAGCAATTTTCATATGCAACTATGCGCCAGGAGGAACACTGACGAGAAGACCTTATGAACC
AGGAATATTTTGTACTCGATGTGGCAGACGTGACAAATGCACAGATTTTCTATGCAGTAATGCAGATC
GTGACCAAGCCACATATTACCGATTTTGGTATCCAAAATGGGAAATGCCCCGGCCAGTTGTGTGTGAT
CCACTGTGCACATTCATTTTATTATTGAGAATATTATGTTTTATCCTGTGTGTCATAACTGTTTTGAT
AGTACAGTCTCAGTTTCCAAATATCTTGTTGGAACAACAAATGATATTTACCCCTGAGGAATCTGAAG
CAGGGAATGAAGAGGAGGAAAAAGAGGAAGAGAAGAAAGAGAAAGAGGAAATGGAAATGGAAATAATG
GAAATGGAGGAGGAAAAAGAAGAGAGAGAGGAGGAGGAGGAGGAAACACAAAAAGAAAAGATGGAGGA
AGAGGAAAAATAAGAGTAGAAAGAGGAGGAAAAAGATGTATCACCAATATAAACCAAAAGTGTAATAC
AAAAAAAGACAGAAAAAAAAAAAAAGTAAAACACTGAGTTTTAACAAGAAAGAAAATATGCAAACCAC
CATTGGAATGTTTTT

| | | | |
|---|---|---|---|
| NOV55a, CG59932-01 | SEQ ID NO: 1122 | 343 aa | MW at 40047.3kD |
| Protein Sequence | | | |

EAARPFAREWRAQSLPLAVGGVLKLRLCELWLLLLGSSLNARFLPDEEDVDFINEYVNLHNELRGDVI
HAEKKMYNFENGSCSGDCSNYIQLVWDHSYKVGCAVTPCSKIGHIIHAAIFICNYAPGGTLTRRPYEP
GIFCTRCGRRDKCTDFLCSNADRDQATYYRFWYPKWEMPRPVVCDPLCTFILLLRILCFILCVITVLI
VQSQFPNILLEQQMIFTPEESEAGNEEEEKEEEKKEKEEMEMEIMEMEEEKEEREEEEEETQKEKMEE
EEK

Further analysis of the NOV55a protein yielded the following properties shown in Table 55B.

TABLE 55B
Protein Sequence Properties NOV55a

| | |
|---|---|
| SignalP analysis: | Cleavage site between residues 42 and 43 |
| PSORT II analysis: | |

| PSG: | a new signal peptide prediction method | |
|---|---|---|
| | N-region: | length 11; pos. chg 3; neg. chg 2 |
| | H-region: | length 12; peak value 7.44 |
| | PSG score: | 3.04 |

| | |
|---|---|
| GvH: | von Heijne's method for signal seq. recognition |
| | GvH score (threshold: −2.1): −0.10 |
| | possible cleavage site: between 41 and 42 |

>>> Seems to have a cleavable signal peptide (1 to 41)

| ALOM: | Klein et al's method for TM region allocation | | |
|---|---|---|---|
| | Init position for calculation: 42 | | |
| | Tentative number of TMS(s) for the threshold 0.5: 1 | | |
| | Number of TMS(s) for threshold 0.5: 1 | | |
| | INTEGRAL | Likelihood = | Transmembrane 257-273 |
| | | −13.43 | |

| | |
|---|---|
| PERIPHERAL | Likelihood = 2.70 (at 172) |

| | |
|---|---|
| | ALOM score: −13.43 (number of TMSs: 1) |
| MTOP: | Prediction of membrane topology (Hartmann et al.) |
| | Center position for calculation: 20 |
| | Charge difference: −1.0 C(1.0)-N(2.0) |
| | N >= C: N-terminal side will be inside |

>>> membrane topology: type 1a (cytoplasmic tail 274 to 343)

| MITDISC: | discrimination of mitochondrial targeting seq | | | |
|---|---|---|---|---|
| | R content: | 4 | Hyd Moment(75): | 9.56 |
| | Hyd Moment(95): | 18.17 | G content: | 2 |
| | D/E content: | 3 | S/T content: | 1 |

| | |
|---|---|
| | Score: −4.20 |
| Gavel: | prediction of cleavage sites for mitochondrial preseq |
| | cleavage site motif not found |

NUCDISC: discrimination of nuclear localization signals

| | |
|---|---|
| | pat4: none |
| | pat7: none |
| | bipartite: none |
| | content of basic residues: 11.4% |
| | NLS Score: −0.47 |
| KDEL: | ER retention motif in the C-terminus: none |

ER Membrane Retention Signals:

| | |
|---|---|
| | XXRR-like motif in the N-terminus: AARP |
| none | |

SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: found

LL at 281

checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 94.1 |
| COIL: | Lupas's algorithm to detect coiled-coil regions |
| 290 E | 1.00 |
| 291 E | 1.00 |
| 292 S | 1.00 |
| 293 E | 1.00 |
| 294 A | 1.00 |
| 295 G | 1.00 |
| 296 N | 1.00 |
| 297 E | 1.00 |
| 298 E | 1.00 |
| 299 E | 1.00 |
| 300 E | 1.00 |
| 301 K | 1.00 |
| 302 E | 1.00 |
| 303 E | 1.00 |
| 304 E | 1.00 |
| 305 K | 1.00 |
| 306 K | 1.00 |
| 307 E | 1.00 |
| 308 K | 1.00 |
| 309 E | 1.00 |
| 310 E | 1.00 |
| 311 M | 1.00 |
| 312 E | 1.00 |
| 313 M | 1.00 |
| 314 E | 1.00 |
| 315 I | 1.00 |
| 316 M | 1.00 |
| 317 E | 1.00 |
| 318 M | 1.00 |
| 319 E | 1.00 |
| 320 E | 1.00 |
| 321 E | 1.00 |
| 322 K | 1.00 |
| 323 E | 1.00 |
| 324 E | 1.00 |
| 325 R | 1.00 |
| 326 E | 1.00 |
| 327 E | 1.00 |
| 328 E | 1.00 |
| 329 E | 1.00 |
| 330 E | 1.00 |
| 331 E | 1.00 |
| 332 T | 1.00 |
| 333 Q | 1.00 |
| 334 K | 1.00 |
| 335 E | 1.00 |
| 336 K | 1.00 |
| 337 M | 1.00 |
| 338 E | 1.00 |
| 339 E | 1.00 |
| 340 E | 1.00 |
| 341 E | 1.00 |
| 342 K | 1.00 |
| | total: 53 residues |

Final Results (k = 9/23):

55.6%: endoplasmic reticulum
22.2%: Golgi
11.1%: plasma membrane
11.1%: extracellular, including cell wall

>> prediction for CG59932-01 is end (k = 9)

A search of the NOV55a protein against the Geneseq database, a proprietary database that contains sequences published in patents and patent publication, yielded several homologous proteins shown in Table 55C.

TABLE 55C
Geneseq Results for NOV55a

| | | NOV55a | Identities/ | | |
|---|---|---|---|---|---|
| | | Residues/ | Similarities for | | |
| Geneseq | Protein/Organism/Length | Match | the Matched | | Expect |
| Identifier | [Patent #, Date] | Residues | Region | | Value |
| ABP69108 | Human polypeptide SEQ ID NO 1155 - | 1 . . . 223 | 220/223 | (98%) | e−134 |
| | Homo sapiens, 253 aa. [WO200270539- | 2 . . . 224 | 222/223 | (98%) | |
| | A2, 12-SEP-2002] | | | | |
| AAB64953 | Human secreted protein sequence | 45 . . . 222 | 177/178 | (99%) | e−109 |
| | encoded by gene 12 SEQ ID NO: 131 - | 1 . . . 178 | 177/178 | (99%) | |
| | Homo sapiens, 178 aa. [WO200076530- | | | | |
| | A1, 21-DEC-2000] | | | | |
| AAB43408 | Human cancer associated protein | 38 . . . 282 | 102/250 | (40%) | 6e−51 |
| | sequence SEQ ID NO: 853 - Homo | 55 . . . 300 | 143/250 | (56%) | |
| | sapiens, 302 aa. [WO200055350-A1, | | | | |
| | 21-SEP-2000] | | | | |
| ABB53121 | Human ORF27 protein - Homo sapiens, | 94 . . . 178 | 85/85 | (100%) | 2e−48 |
| | 85 aa. [WO200177155-A2, 18-OCT-2001] | 1 . . . 85 | 85/85 | (100%) | |
| AAE18962 | Mouse testes-specific, vespid and | 37 . . . 282 | 96/247 | (38%) | 1e−46 |
| | pathogenic protein (RTVP) - Mus sp, | 18 . . . 253 | 138/247 | (55%) | |
| | 255 aa. [WO200206344-A2, 24-JAN-2002] | | | | |

In a BLAST search of public sequence databases, the NOV55a protein was found to have homology to the proteins shown in the BLASTP data in Table 55D.

TABLE 55D
Public BLASTP Results for NOV55a

| | | | Identities/ | |
|---|---|---|---|---|
| Protein | | NOV55a | Similarities | |
| Accession | | Residues/ | for the | Expect |
| Number | Protein/Organism/Length | Match Residues | Matched Portion | Value |
| Q8NA43 | Hypothetical protein FLJ35856 - | 1 . . . 228 | 228/228 (100%) | e−139 |
| | Homo sapiens (Human), 229 aa. | 2 . . . 229 | 228/228 (100%) | |
| Q8N6N0 | Similar to RIKEN cDNA 4921508O11 | 1 . . . 223 | 223/223 (100%) | e−135 |
| | gene - Homo sapiens (Human), 253 | 2 . . . 224 | 223/223 (100%) | |
| | aa. | | | |
| Q9CQ35 | 4921508O11 Rik protein - Mus | 1 . . . 343 | 209/345 (60%) | e−117 |
| | musculus (Mouse), 332 aa. | 2 . . . 331 | 252/345 (72%) | |
| P48060 | Glioma pathogenesis-related protein | 38 . . . 282 | 103/250 (41%) | 1e−51 |
| | (GliPR) (RTVP-1 protein) - Homo | 19 . . . 264 | 144/250 (57%) | |
| | sapiens (Human), 266 aa. | | | |
| JC5308 | testis-specific, vespid, and | 38 . . . 282 | 103/250 (41%) | 1e−51 |
| | pathogenesis-related protein 1 | 19 . . . 264 | 144/250 (57%) | |
| | precursor - human, 266 aa. | | | |

PFam analysis predicts that the NOV55a protein contains the domains shown in the Table 55E.

TABLE 55E
Domain Analysis of NOV55a

| | | Identities/ | |
|---|---|---|---|
| Pfam | NOV55a | Similarities for | Expect |
| Domain | Match Region | the Matched Region | Value |
| SCP | 57 . . . 190 | 42/167 (25%) | 3.1e−33 |
| | | 95/167 (57%) | |

### Example 56

The NOV56 clone was analyzed, and the nucleotide and encoded polypeptide sequences are shown in Table 56A.

TABLE 56A
NOV56 Sequence Analysis

| | | |
|---|---|---|
| NOV56a, CG92715-01 | SEQ ID NO: 1123 | 2800 bp |
| DNA Sequence | ORF Start: ATG at 26 | ORF Stop: end of sequence |

TAGACGCGGAGCCCAAGGAGGTAAAATGCACACTTGCTGCCCCCCAGTAACTTTGGAACAGGACCTTC
ACAGAAAAATGCATAGCTGGATGCTGCAGACTCTAGCGTTTGCTGTAACATCTCTCGTCCTTTCGTGT
GCAGAAACCATCGATTATTACGGGGAAATCTGTGACAATGCATGTCCTTGTGAGGAAAAGGACGGCAT
TTTAACTGTGAGCTGTGAAAACCGGGGGATCATCAGTCTCTCTGAAATTAGCCCTCCCCGTTTCCCAA
TCTACCACCTCTTGTTGTCCGGAAACCTTTTGAACCGTCTCTATCCCAATGAGTTTGTCAATTACACT
GGGGCTTCAATTTTGCATCTAGGTAGCAATGTTATCCAGGACATTGAGACCGGGGCTTTCCATGGGCT
ACGGGGTTTGAGGAGATTGCATCTAAACAATAATAAACTGGAACTTCTGCGAGATGATACCTTCCTTG
GCTTGGAGAACCTGGAGTACCTACAGGTCGATTACAACTACATCAGCGTCATTGAACCCAATGCTTTT
GGGAAACTGCATTTGTTGCAGGTGCTTATCCTCAATGACAATCTTTTGTCCAGTTTACCCAACAATCT
TTTCCGTTTTGTGCCCTTAACGCACTTGGACCTCCGGGGGAACCGGCTGAAACTTCTGCCCTACGTGG
GGCTCTTGCAGCACATGGATAAAGTTGTGGAGCTACAGCTGGAGGAAAACCCTTGGAATTGTTCTTGT
GAGCTGATCTCTCTAAAGGATTGGTTGGACAGCATCTCCTATTCAGCCCTGGTGGGGGATGTAGTTTG
TGAGACCCCCTTCCGCTTACACGGAAGGGACTTGGACGAGGTATCCAAGCAGGAACTTTGCCCAAGGA
GACTTATTTCTGACTACGAGATGAGGCCGCAGACGCCTTTGAGCACCACGGGGTATTTACACACCACC
CCGGCGTCAGTGAATTCTGTGGCCACTTCTTCCTCTGCTGTTTACAAACCCCCTTTGAAGCCCCCTAA
GGGGACTCGCCAACCCAACAAGCCCAGGGTGCGCCCCACCTCTCGGCAGCCCTCTAAGGACTTGGGCT
ACAGCAACTATGGCCCCAGCATCGCCTATCAGACCAAATCCCCGGTGCCTTTGGAGTGTCCCACCGCG
TGCTCTTGCAACCTGCAGATCTCTGATCTGGGCCTCAACGTAAACTGCCAGGAGCGAAAGATCGAGAG
CATCGCTGAACTGCAGCCCAAGCCCTACAATCCCAAGAAAATGTATCTGACAGAGAACTACATCGCTG
TCGTGCGCAGGACAGACTTCCTGGAGGCCACGGGGCTGGACCTCCTGCACCTGGGGAATAACCGCATC
TCGATGATCCAGGACCGCGCTTTCGGGGATCTCACCAACCTGAGGCGCCTCTACCTGAATGGCAACAG
GATCGAGAGGCTGAGCCCGGAGTTATTCTATGGCCTGCAGAGCCTGCAGTATCTCTTCCTCCAGTACA
ATCTCATCCGCGAGATTCAGTCTGGAACTTTTGACCCGGTCCCAAACCTCCAGCTGCTATTCTTGAAT
AACAACCTCCTGCAGGCCATGCCCTCAGGCGTCTTCTCTGGCTTGACCCTCCTCAGGCTAAACCTGAG
GAGTAACCACTTCACCTCCTTGCCAGTGAGTGGAGTTTTGGACCAGCTGAAGTCACTCATCCAAATCG
ACCTGCATGACAATCCTTGGGATTGTACCTGTGACATTGTGGGCATGAAGCTGTGGGTGGAGCAGCTC
AAAGTGGGCGTCCTAGTGGACGAGGTGATCTGTAAGGCGCCCAAAAAATTCGCTGAGACCGACATGCG
CTCCATTAAGTCGGAGCTGCTGTGCCCTGACTATTCAGATGTAGTAGTTTCCACGCCCACACCCTCCT
CTATCCAGGTCCCTGCGAGGACCAGCGCCGTGACTCCTGCGGTCCGGTTGAATAGCACCGGGGCCCCC
GCGAGCTTGGGCGCAGGCGGAGGGGCGTCGTCGGTGCCCTTGTCTGTGTTAATTCTCAGCCTCCTGCT
GGTTTTCATCATGTCCGTCTTCGTGGCCGCCGGGCTCTTCGTGCTGGTCATGAAGCGCAGGAAGAAGA
ACCAGAGCGACCACACCAGCACCAACAACTCCGACGTGAGCTCCTTTAACATGCAGTACAGCGTGTAC
GGCGGCGGCGGCGGCACGGGCGGCCACCCACACGCGCACGTGCATCACCGCGGGCCCGCGCTGCCCAA
GGTGAAGACGCCCGCGGGCCACGTGTATGAATACATCCCCCACCCACTGGGCCACATGTGCAAAAACC
CCATCTACCGCTCCCGAGAGGGCAACTCCGTAGAGGATTACAAAGACCTGCACGAGCTCAAGGTCACC
TACAGCAGCAACCACCACCTGCAGCAGCAGCAGCAGCCGCCGCCGCCACCGCAGCAGCCACAGCAGCA
GCCCCCGCCGCAGCTGCAGCTGCAGCCTGGGGAGGAGGAGAGGCGGGAAAGCCACCACTTGCGGAGCC
CCGCCTACAGCGTCAGCACCATCGAGCCCCGGGAGGACCTGCTGTCGCCGGTGCAGGACGCCGACCGC
TTTTACAGGGGCATTTTAGAACCAGACAAACACTGCTCCACCACCCCCGCCGGCAATAGCCTCCCGGA
ATATCCCAAATTCCCGTGCAGCCCCGCTGCTTACACTTTCTCCCCCAACTATGACCTGAGACGCCCCC
ATCAGTATTTGCACCCGGGGGCAGGGGACAGCAGGCTACGGGAACCGGTGCTCTACAGCCCCCCGAGT
GCTGTCTTTGTA

| | | | |
|---|---|---|---|
| NOV56a, CG92715-01 | SEQ ID NO: 1124 | 925 aa | MW at 103516.1kD |
| Protein Sequence | | | |

MHTCCPPVTLEQDLHRKMHSWMLQTLAFAVTSLVLSCAETIDYYGEICDNACPCEEKDGTLTVSCENR
GIISLSEISPPRFPIYHLLLSGNLLNRLYPNEFVNYTGASILHLGSNVIQDIETGAFHGLRGLRRLHL
NNNKLELLRDDTFLGLENLEYLQVDYNYISVIEPNAFGKLHLLQVLILNDNLLSSLPNNLFRFVPLTH
LDLRGNRLKLLPYVGLLQHMDKVVELQLEENPWNCSCELISLKDWLDSISYSALVGDVVCETPFRLHG
RDLDEVSKQELCPRRLISDYEMRPQTPLSTTGYLHTTPASVNSVATSSSAVYKPPLKPPKGTRQPNKP
RVRPTSRQPSKDLGYSNYGPSIAYQTKSPvPLECPTACSCNLQISDLGLNVNCQERKIESIAELQPKP
YNPKKMYLTENYIAVVRRTDFLEATGLDLLHLGNNRISMIQDRAFGDLTNLRRLYLNGNRIERLSPEL
FYGLQSLQYLFLQYNLIREIQSGTFDPVPNLQLLFLNNNLLQAMPSGVFSGLTLLRLNLRSNHFTSLP
VSGVLDQLKSLIQIDLHDNPWDCTCDIVGMKLWVEQLKVGVLVDEVICKAPKKFAETDMRSIKSELLC
PDYSDVVVSTPTPSSIQVPARTSAVTPAVRLNSTGAPASLGAGGGASSVPLSVLILSLLLVFIMSVFV
AAGLFVLVMKRRKKNQSDHTSTNNSDVSSFNMQYSVYGGGGGTGGHPHAHVHHRGPALPKVKTPAGHV
YEYIPHPLGHMCKNPIYRSREGNSVEDYKDLHELKVTYSSNHHLQQQQQPPPPPQQPQQQPPPQLQLQ
PGEEERRESHHLRSPAYSVSTIEPREDLLSPVQDADRFYRGILEPDKHCSTTPAGNSLPEYPKFPCSP
AAYTFSPNYDLRRPHQYLHPGAGDSRLREPVLYSPPSAVFV

| | | |
|---|---|---|
| NOV56b, 248576233 | SEQ ID NO: 1125 | 1872 bp |
| DNA Sequence | ORF Start: at 1 | ORF Stop: end of sequence |

GGATCCATCGATTATTACGGGGAAATCTGTGACAATGCATGTCCTTGTGAGGAAAAGGACGGCATTTT
AACTGTGAGCTGTGAAAACCGGGGGATCATCAGTCTCTCTGAAATTAGCCCTCCCCGTTTCCCAATCT
ACCACCTCTTGTTGTCCGGAAACCTTTTGAACCGTCTCTATCCCAATGAGTTTGTCAATTACACTGGG
GCTTCAATTTTGCATCTAGGTAGCAATGTTATCCAGGACATTGAGACCGGGGCTTTCCATGGGCTACG
GGGTTTGAGGAGATTGCATCTAAACAATAATAAACTGGAACTTCTGCGAGATGATACCTTCCTTGGCT
TGGAGAACCCGGAGTACCTACAGGTCGATTACAACTACATCAGCGTCATTGAACCCAATGCTTTTGGG
AAACTGCATTTGTTGCAGGTGCTTATCCTCAATGACAATCTTTTGTCCAGTTTACCCAACAATCTTTT
CCGTTTTGTGCCCTTAACGCACTTGGACCTCCGGGGGAACCGGCTGAAACTTCTGCCCTACGTGGGGC
TCTTGCAGCACATGGATAAAGTTGTGGAGCTACAGCTGGAGGAAAACCCTTGGAATTGTTCTTGTGAG
CTGATCTCTCTAAAGGATTGGTTGGACAGCATCTCCTATTCAGCCCTGGTGGGGGATGTAGTTTGTGA
GACCCCCTTCCGCTTACACGGAAGGGACTTGGACGAGGTATCCAAGCAGGAACTTTGCCCAAGGAGAC
TTATTTCTGACTACGAGATGAGGCCGCAGACGCCTTTGAGCACCACGGGGTATTTACACACCACCCCG
GCGTCAGTGAATTCTGTGGCCACTTCTTCCTCTGCTGTTTACAAACCCCCTTTGAAGCCCCCTAAGGG
GACTCGCCAACCCAACAAGCCCAGGGTGCGCCCCACCTCTCGGCAGCCCTCTAAGGACTTGGGCTACA
GCAACTATGGCCCCAGCATCGCCTATCAGACCAAATCCCCGGTGCCTTTGGAGTGTCCCACCGCGTGC
TCTTGCAACCTGCAGATCTCTGATCTGGGCCTCAACGTAAACTGCCAGGAGCGAAAGATCGAGAGCAT
CGCTGAACTGCAGCCCAAGCCCTACAATCCCAAGAAAATGTATCTGACAGAGAACTACATCGCTGTCG
TGCGCAGGACAGACTTCCTGGAGGCCACGGGGCTGGACCTCCTGCACCTGGGGAATAACCGCATCTCG
ATGATCCAGGACCGCGCTTTCGGGGATCTCACCAACCTGAGGCGCCTCTACCTGAATGGCAACAGGAT
CGAGAGGCTGAGCCCGGAGTTATTCTATGGCCTGCAGAGCCTGCAGTATCTCTTCCTCCAGTACAATC
TCATCCGCGAGATTCAGTCTGGAACTTTTGACCCGGTCCCAAACCTCCAGCTGCTATTCTTGAATAAC
AACCTCCTGCAGGCCATGCCCTCAGGCGTCTTCTCTGGCTTGACCCTCCTCAGGCTAAACCTGAGGAG
TAACCACTTCACCTCCTTGCCAGTGAGTGGAGTTTTGGACCAGCTGAAGTCACTCATCCAAATCGACC
TGCATGACAATCCTTGGGATTGTACCTGTGACATTGTGGGCATGAAGCTGTGGGTGGAGCAGCTCAAA
GTGGGCGTCCTAGTGGACGAGGTGATCTGTAAGGCGCCCAAAAAATTCGCTGAGACCGACATGCGCTC
CATTAAGTCGGAGCTGCTGTGCCCTGACTATTCAGATGTAGTAGTTTCCACGCCCACACCCTCCTCTA
TCCAGGTCCCTGCGAGGACCAGCGCCGTGACTCCTGCGGTCCGGTTGAATAGCACCGGGGCCCCCGCG
AGCTTGGGCGCAGGCGGAGGGGCGTCGTCGCTCGAG

| | | | |
|---|---|---|---|
| NOV56b, 248576233 | SEQ ID NO: 1126 | 624 aa | MW at 69694.9kD |
| Protein Sequence | | | |

GSIDYYGEICDNACPCEEKDGILTVSCENRGIISLSEISPPRFPIYHLLLSGNLLNRLYPNEFVNYTG
ASILHLGSNVIQDIETGAFHGLRGLRRLHLNNNKLELLRDDTFLGLENPEYLQVDYNYISVIEPNAFG
KLHLLQVLILNDNLLSSLPNNLFRFVPLTHLDLRGNRLKLLPYVGLLQHMDKVVELQLEENPWNCSCE
LISLKDWLDSISYSALVGDVVCETPFRLHGRDLDEVSKQELCPRRLISDYEMRPQTPLSTTGYLHTTP
ASVNSVATSSSAVYKPPLKPPKGTRQPNKPRVRPTSRQPSKDLGYSNYGPSIAYQTKSPVPLECPTAC
SCNLQISDLGLNVNCQERKIESIAELQPKPYNPKKMYLTENYIAVVRRTDFLEATGLDLLHLGNNRIS
MIQDRAFGDLTNLRPLYLNGNRIERLSPELFYGLQSLQYLFLQYNLIREIQSGTFDPVPNLQLLFLNN
NLLQAMPSGVFSGLTLLRLNLRSNHFTSLPVSGVLDQLKSLIQIDLHDNPWDCTCDIVGMKLWVEQLK
VGVLVDEVICKAPKKFAETDMRSIKSELLCPDYSDVVVSTPTPSSIQVPARTSAVTPAVRLNSTGAPA
SLGAGGGASSLE

| | | |
|---|---|---|
| NOV56c, CG92715-02 | SEQ ID NO: 1127 | 4500 bp |
| DNA Sequence | ORF Start: ATG at 178 | ORF Stop: TAA at 3094 |

CGGAACCCGCGGTCGCCACCGCGGCGGCGGCCCCAGGCTGGAGGCGTCCGGGCGCCTCTTTCCTCCAG
CCTCTGGGACTGCGCTGCTCGCAGTCTCCTCGCCCTGCCTGGGCTTGAGAAACCTAGTGCATACCCCA
AAGAGGGTTTTTGTGTATGTGTGTGTTTTTAAAGGGTGGCTATGATGACTGGGCCTTGGAGACGCGGA
GACCAAGGAGGTAAAATGCACACTTGCTGCCCCCCAGTAACTTTGGAACAGGACCTTCACAGAAAAAT
GCATAGCTGGATGCTGCAGACTCTAGCGTTTGCTGTAACATCTCTCGTCCTTTCGTGTGCAGAAACCA
TCGATTATTACGGGGAAATCTGTGACAATGCATGTCCTTGTGAGGAAAAGGACGGCATTTTAACTGTG
AGCTGTGAAAACCGGGGGATCATCAGTCTCTCTGAAATTAGCCCTCCCCGTTTCCCAATCTACCACCT
CTTGTTGTCCGGAAACCTTTTGAACCGTCTCTATCCCAATGAGTTTGTCAATTACACTGGGGCTTCAA
TTTTGCATCTAGGTAGCAATGTTATCCAGGACATTGAGACCGGGGCTTTCCATGGGCTACGGGGTTTG
AGGAGATTGCATCTAAACAATAATAAACTGGAACTTCTGCGAGATGATACCTTCCTTGGCTTGGAGAA
CCTGGAGTACCTACAGGTCGATTACAACTACATCAGCGTCATTGAACCCAATGCTTTTGGGAAACTGC
ATTTGTTGCAGGTGCTTATCCTCAATGACAATCTTTTGTCCAGTTTACCCAACAATCTTTTCCGTTTT
GTGCCCTTAACGCACTTGGACCTCCGGGGGAACCGGCTGAAACTTCTGCCCTACGTGGGGCTCTTGCA
GCACATGGATAAAGTTGTGGAGCTACAGCTGGAGGAAAACCCTTGGAATTGTTCTTGTGAGCTGATCT
CTCTAAAGGATTGGTTGGACAGCATCTCCTATTCAGCCCTGGTGGGGGATGTAGTTTGTGAGACCCCC
TTCCGCTTACACGGAAGGGACTTGGACGAGGTATCCAAGCAGGAACTTTGCCCAAGGAGACTTATTTC
TGACTACGAGATGAGGCCGCAGACGCCTTTGAGCACCACGGGGTATTTACACACCACCCCGGCGTCAG
TGAATTCTGTGGCCACTTCTTCCTCTGCTGTTTACAAACCCCCTTTGAAGCCCCCTAAGGGGACTCGC
CAACCCAACAAGCCCAGGGTGCGCCCCACCTCTCGGCAGCCCTCTAAGGACTTGGGCTACAGCAACTA
TGGCCCCAGCATCGCCTATCAGACCAAATCCCCGGTGCCTTTGGAGTGTCCCACCGCGTGCTCTTGCA
ACCTGCAGATCTCTGATCTGGGCCTCAACGTAAACTGCCAGGAGCGAAAGATCGAGAGCATCGCTGAA
CTGCAGCCCAAGCCCTACAATCCCAAGAAAATGTATCTGACAGAGAACTACATCGCTGTCGTGCGCAG
GACAGACTTCCTGGAGGCCACGGGGCTGGACCTCCTGCACCTGGGGAATAACCGCATCTCGATGATCC
AGGACCGCGCTTTCGGGGATCTCACCAACCTGAGGCGCCTCTACCTGAATGGCAACAGGATCGAGAGG
CTGAGCCCGGAGTTATTCTATGGCCTGCAGAGCCTGCAGTATCTCTTCCTCCAGTACAATCTCATCCG
CGAGATTCAGTCTGGAACTTTTGACCCGGTCCCAAACCTCCAGCTGCTATTCTTGAATAACAACCTCC
TGCAGGCCATGCCCTCAGGCGTCTTCTCTGGCTTGACCCTCCTCAGGCTAAACCTGAGGAGTAACCAC
TTCACCTCCTTGCCAGTGAGTGGAGTTTTGGACCAGCTGAAGTCACTCATCCAAATCGACCTGCATGA
CAATCCTTGGGATTGTACCTGTGACATTGTGGGCATGAAGCTGTGGGTGGAGCAGCTCAAAGTGGGCG
TCCTAGTGGACGAGGTGATCTGTAAGGCGCCCAAAAAATTCGCTGAGACCGACATGCGCTCCATTAAG
TCGGAGCTGCTGTGCCCTGACTATTCAGATGTAGTAGTTTCCACGCCCACACCCTCCTCTATCCAGGT
CCCTGCGAGGACCAGCGCCGTGACTCCTGCGGTCCGGTTGAATAGCACCGGGGCCCCCGCGAGCTTGG
GCGCAGGCGGAGGGGCGTCGTCGGTGCCCTTGTCTGTGTTAATTCTCAGCCTCCTGCTGGTTTTCATC
ATGTCCGTCTTCGTGGCCGCCGGGCTCTTCGTGCTGGTCATGAAGCGCAGGAAGAAGAACCAGAGCGA
CCACACCAGCACCAACAACTCCGACGTGAGCTCCTTTAACATGCAGTACAGCGTGTACGGCGGCGGCG
GCGGCACGGGCGGCCACCCACACGCGCACGTGCATCACCGCGGGCCCGCGCTGCCCAAGGTGAAGACG
CCCGCGGGCCACGTGTATGAATACATCCCCCACCCACTGGGCCACATGTGCAAAAACCCCATCTACCG
CTCCCGAGAGGGCAACTCCGTAGAGGATTACAAAGACCTGCACGAGCTCAAGGTCACCTACAGCAGCA
ACCACCACCTGCAGCAGCAGCAGCAGCCGCCGCCGCCACCGCAGCAGCCACAGCAGCAGCCCCCGCCG
CAGCTGCAGCTGCAGCCCGGGGAGGAGGAGAGGCGGGAAAGCCACCACTTGCGGAGCCCCGCCTACAG
CGTCAGCACCATCGAGCCCCGGGAGGACCTGCTGTCGCCGGTGCAGGACGCCGACCGCTTTTACAGGG
GCATTTTAGAACCAGACAAACACTGCTCCACCACCCCCGCCGGCAATAGCCTCCCGGAATATCCCAAA
TTCCCGTGCAGCCCCGCTGCTTACACTTTCTCCCCCAACTATGACCTGAGACGCCCCCATCAGTATTT
GCACCCGGGGGCAGGGGACAGCAGGCTACGGGAACCGGTGCTCTACAGCCCCCCGAGTGCTGTCTTTG
TAGAACCCAACCGGAACGAATATCTGGAGTTAAAAGCAAAACTAAACGTTGAGCCGGACTACCTCGAA
GTGCTGGAAAAACAGACCACGTTTAGCCAGTTCTAAAAGCAAAGAAACTCTCTTGGAGCTTTTGCATT
TAAAACAAACAAGCAAGCAGACACACACAGTGAACACATTTGATTAATTGTGTTGTTTCAACGTTTAG
GGTGAAGTGCCTTGGCACGGGATTTCTCAGCTTCGGTGGAAGATACGAAAAGGGTGTGCAATTTCCTT
TAAAATTTACACGTGGGAAACATTTGTGTAAACTGGGCACATCACTTTCTCTTCTTGCGTGTGGGGCA
GGTGTGGAGAAGGGCTTTAAGGAGGCCAATTTGCTGCGCGGGTGACCTGTGAAAGGTCACAGTCATTT
TTGTAGTGGTTGGAAGTGCTAAGAATGGTGGATGATGGCAGAGCATAGATTCTACTCTTCCTCTTTAG
CTTCCTCCCCATCCAACGAACCCTGCCCAACACTCTAAATATCCACCAGATAAGACATGGAATGAGGT
CTAAATGACACAAAGTGAAGAAATCAACACAACACAAACTTTACAGCTAACAACAAATGATCAACAAA
AACCGAACCAACAAGACAACCATCGAACCTCACCACTCCACACTCACAACAACTCATATCAAGACAAC
AACACAATGACGTTAAAGGAAACGAAATCAATGCAAAAATAGACATTTGACAATACAAAAAAACAAGA
ACCGTGATCACACTACAACCGAAGCAACCATAGATGTGAGAAAAAACAACAAACAAAACACCGAGCTA
TATGATCCATAATTGATTAGTCAAAATAACTTATTGATGAAATATACAAATATTTTATTGTAGCACCT
ATTTTTATATGCACATTTAGCATTCCTCTTTCCTTCACTATTTAGCCTATGATTTTGCAGAGGTGTCA
CACTGTATTAGGATCTGCATTTCTAAAACTGACGTGGTATCAGGAAGGCATTTTCAATCATTCAAAAT
GTGGAGAATTTAATGGCTAAATCTTTAAAAGCCAATGCAACCCACCCAATTGAATCTGCATTTTCTTT
TAAGAAAACAGAGCTGATTGTATCCCAATGTATTTTAAAAAATAGGGCAATTGATTGGGCCATTCCGA
GAGAATTGTTTGCAAGTTTTGGGTTTTATTAGAAAATATTTGAAAGTATTTTTATTAATGAACCAAAA
TGACATGTTCATTTGACTACTATTGTAGCCGATTTTCGATTGTTTAACCAAACCCAGTTGCATTTGTA
CAGATCCACGTGTACTGGCACCTCAGAAGACCAAATCATGGACTGTACAAGTCTCTATACAATGTCTT
TATCCCTGTGGGCAGCAAGCAATGATGATAATGACAAACAGGATATCTGTAAGATGGGGCTACTGTTG
TTACAGTCTCATATGTATCCCAGCACATGTAATTTTTTAAATAGTTTCTGAATAAACACTTGATAACT
ATGTCAAAAAAA

| | | | |
|---|---|---|---|
| NOV56c, CG92715-02 | SEQ ID NO: 1128 | 972 aa | MW at 109043.3kD |
| Protein Sequence | | | |

MMTGPWRRGDQGGKMHTCCPPVTLEQDLHRKMHSWMLQTLAFAVTSLVLSCAETIDYYGEICDNACPC
EEKDGILTVSCENRGIISLSEISPPRFPIYHLLLSGNLLNRLYPNEFVNYTGASILHLGSNVIQDIET
GAFHGLRGLRRLHLNNNKLELLRDDTFLGLENLEYLQVDYNYISVIEPNAFGKLHLLQVLILNDNLLS
SLPNNLFRFVPLTHLDLRGNRLKLLPYVGLLQHNDKVVELQLEENPWNCSCELISLKDWLDSISYSAL
VGDVVCETPFRLHGRDLDEVSKQELCPRRLISDYEMRPQTPLSTTGYLHTTPASVNSVATSSSAVYKP
PLKPPKGTRQPNKPRVRPTSRQPSKDLGYSNYGPSIAYQTKSPVPLECPTACSCNLQISDLGLNVNCQ
ERKIESIAELQPKPYNPKKNYLTENYIAVVRRTDFLEATGLDLLHLGNNRTSMIQDRAFGDLTNLRRL
YLNGNRIERLSPELFYGLQSLQYLFLQYNLIREIQSGTFDPVPNLQLLFLNNNLLQAMPSGVFSGLTL
LRLNLRSNHFTSLPVSGVLDQLKSLIQIDLHDNPWDCTCDIVGMKLWVEQLKVGVLVDEVICKAPKKF
AETDMRSIKSELLCPDYSDVVVSTPTPSSIQVPARTSAVTPAVRLNSTGAPASLGAGGGASSVPLSVL
ILSLLLVFIMSVFVAAGLFVLVMKRRKKNQSDHTSTNNSDVSSFNMQYSVYGGGGGTGGHPHAHVHHR
GPALPKVKTPAGHVYEYIPHPLGHMCKNPIYRSREGNSVEDYKDLHELKVTYSSNHHLQQQQQPPPPP
QQPQQQPPPQLQLQPGEEERRESHHLRSPAYSVSTIEPREDLLSPVQDADRFYRGILEPDKHCSTTPA
GNSLPEYPKFPCSPAAYTFSPNYDLRRPHQYLHPGAGDSRLREPVLYSPPSAVFVEPNRNEYLELKAK
LNVEPDYLEVLEKQTTFSQF

| | | |
|---|---|---|
| NOV56d, CG92715-03 | SEQ ID NO: 1129 | 1872 bp |
| DNA Sequence | ORF Start: at 7 | ORF Stop: at 1867 |

GGATCCATCGATTATTACGGGGAAATCTGTGACAATGCATGTCCTTGTGAGGAAAAGGACGGCATTTT
AACTGTGAGCTGTGAAAACCGGGGGATCATCAGTCTCTCTGAAATTAGCCCTCCCCGTTTCCCAATCT
ACCACCTCTTGTTGTCCGGAAACCTTTTGAACCGTCTCTATCCCAATGAGTTTGTCAATTACACTGGG
GCTTCAATTTTGCATCTAGGTAGCAATGTTATCCAGGACATTGAGACCGGGGCTTTCCATGGGCTACG
GGGTTTGAGGAGATTGCATCTAAACAATAATAAACTGGAACTTCTGCGAGATGATACCTTCCTTGGCT
TGGAGAACCTGGAGTACCTACAGGTCGATTACAACTACATCAGCGTCATTGAACCCAATGCTTTTGGG
AAACTGCATTTGTTGCAGGTGCTTATCCTCAATGACAATCTTTTGTCCAGTTTACCCAACAATCTTTT
CCGTTTTGTGCCCTTAACGCACTTGGACCTCCGGGGGAACCGGCTGAAACTTCTGCCCTACGTGGGGC
TCTTGCAGCACATGGATAAAGTTGTGGAGCTACAGCTGGAGGAAAACCCTTGGAATTGTTCTTGTGAG
CTGATCTCTCTAAAGGATTGGTTGGACAGCATCTCCTATTCAGCCCTGGTGGGGGATGTAGTTTGTGA
GACCCCCTTCCGCTTACACGGAAGGGACTTGGACGAGGTATCCAAGCAGGAACTTTGCCCAAGGAGAC
TTATTTCTGACTACGAGATGAGGCCGCAGACGCCTTTGAGCACCACGGGGTATTTACACACCACCCCG
GCGTCAGTGAATTCTGTGGCCACTTCTTCCTCTGCTGTTTACAAACCCCCTTTGAAGCCCCCTAAGGG
GACTCGCCAACCCAACAAGCCCAGGGTGCGCCCCACCTCTCGGCAGCCCTCTAAGGACTTGGGCTACA
GCAACTATGGCCCCAGCATCGCCTATCAGACCAAATCCCCGGTGCCTTTGGAGTGTCCCACCGCGTGC
TCTTGCAACCTGCAGATCTCTGATCTGGGCCTCAACGTAAACTGCCAGGAGCGAAAGATCGAGAGCAT
CGCTGAACTGCAGCCCAAGCCCTACAATCCCAAGAAAATGTATCTGACAGAGAACTACATCGCTGTCG
TGCGCAGGACAGACTTCCTGGAGGCCACGGGGCTGGACCTCCTGCACCTGGGGAATAACCGCATCTCG
ATGATCCAGGACCGCGCTTTCGGGGATCTCACCAACCTGAGGCGCCTCTACCTGAATGGCAACAGGAT
CGAGAGGCTGAGCCCGGAGTTATTCTATGGCCTGCAGAGCCTGCAGTATCTCTTCCTCCAGTACAATC
TCATCCGCGAGATTCAGTCTGGAACTTTTGACCCGGTCCCAAACCTCCAGCTGCTATTCTTGAATAAC
AACCTCCTGCAGGCCATGCCCTCAGGCGTCTTCTCTGGCTTGACCCTCCTCAGGCTAAACCTGAGGAG
TAACCACTTCACCTCCTTGCCAGTGAGTGGAGTTTTGGACCAGCTGAAGTCACTCATCCAAATCGACC
TGCATGACAATCCTTGGGATTGTACCTGTGACATTGTGGGCATGAAGCTGTGGGTGGAGCAGCTCAAA
GTGGGCGTCCTAGTGGACGAGGTGATCTGTAAGGCGCCCAAAAAATTCGCTGAGACCGACATGCGCTC
CATTAAGTCGGAGCTGCTGTGCCCTGACTATTCAGATGTAGTAGTTTCCACGCCCACACCCTCCTCTA
TCCAGGTCCCTGCGAGGACCAGCGCCGTGACTCCTGCGGTCCGGTTGAATAGCACCGGGGCCCCCGCG
AGCTTGGGCGCAGGCGGAGGGGCGTCGTCGCTCGAG

| | | | |
|---|---|---|---|
| NOV56d, CG92715-03 | SEQ ID NO: 1130 | 620 aa | MW at 69324.6kD |
| Protein Sequence | | | |

IDYYGEICDNACPCEEKDGILTVSCENRGIISLSEISPPRFPIYHLLLSGNLLNRLYPNEFVNYTGAS
ILHLGSNVIQDIETGAFHGLRGLRRLHLNNNKLELLRDDTFLGLENLEYLQVDYNYISVIEPNAFGKL
HLLQVLILNDNLLSSLPNNLFRFVPLTHLDLRGNRLKLLPYVGLLQHMDKVVELQLEENPWNCSCELI
SLKDWLDSISYSALVGDVVCETPFRLHGRDLDEVSKQELCPRRLISDYEMRPQTPLSTTGYLHTTPAS
VNSVATSSSAVYKPPLKPPKGTRQPNKPRVRPTSRQPSKDLGYSNYGPSIAYQTKSPVPLECPTACSC
NLQISDLGLNVNCQERKIESIAELQPKPYNPKKMYLTENYIAVVRRTDFLEATGLDLLHLGNNRISMI
QDRAFGDLTNLRRLYLNGNRIERLSPELFYGLQSLQYLFLQYNLIREIQSGTFDPVPNLQLLFLNNNL
LQAMPSGVFSGLTLLRLNLRSNHFTSLPVSGVLDQLKSLIQIDLHDNPWDCTCDIVGMKLWVEQLKVG
VLVDEVICKAPKKFAETDMRSIKSELLCPDYSDVVVSTPTPSSIQVPARTSAVTPAVRLNSTGAPASL
GAGGGASS

A ClustalW comparison of the above protein sequences yields the following sequence alignment shown in Table 56B.

TABLE 56B
Comparison of the NOV56 protein sequences.

NOV56b VDEVICKAPKKFAETDMRSIKSELLCPDYSDVVVSTPTPSSIQVPARTSAVTPAVRLNST
NOV56c VDEVICKAPKKFAETDMRSIKSELLCPDYSDVVVSTPTPSSIQVPARTSAVTPAVRLNST
NOV56d VDEVICKAPKKFAETDMRSIKSELLCPDYSDVVVSTPTPSSIQVPARTSAVTPAVRLNST
NOV56a GAPASLGAGGGASSVPLSVLILSLLLVFIMSVFVAAGLFVLVMKRRKKNQSDHTSTNNSD
NOV56b GAPASLGAGGGASSLE--------------------------------------------
NOV56c GAPASLGAGGGASSVPLSVLILSLLLVFIMSVFVAAGLFVLVMKRRKKNQSDHTSTNNSD
NOV56d GAPASLGAGGGASS----------------------------------------------
NOV56a VSSFNMQYSVYGGGGGTGGHPHAHVHHRGPALPKVKTPAGHVYEYIPHPLGHMCKNPIYR
NOV56b ------------------------------------------------------------
NOV56c VSSFNMQYSVYGGGGGTGGHPHAHVHHRGPALPKVKTPAGHVYEYIPHPLGHMCKNPIYR
NOV56d ------------------------------------------------------------
NOV56a SREGNSVEDYKDLHELKVTYSSNHHLQQQQQPPPPPQQPQQQPPPQLQLQPGEEERRESH
NOV56b ------------------------------------------------------------
NOV56c SREGNSVEDYKDLHELKVTYSSNHHLQQQQQPPPPPQQPQQQPPPQLQLQPGEEERRESH
NOV56d ------------------------------------------------------------
NOV56a HLRSPAYSVSTIEPREDLLSPVQDADRFYRGILEPDKHCSTTPAGNSLPEYPKFPCSPAA
NOV56b ------------------------------------------------------------
NOV56c HLRSPAYSVSTIEPREDLLSPVQDADRFYRGILEPDKHCSTTPAGNSLPEYPKFPCSPAA
NOV56d ------------------------------------------------------------
NOV56a YTFSPNYDLRRPHQYLHPGAGDSRLREPVLYSPPSAVFV---------------------
NOV56b ------------------------------------------------------------
NOV56c YTFSPNYDLRRPHQYLHPGAGDSRLREPVLYSPPSAVFVEPNRNEYLELKAKLNVEPDYL
NOV56d ------------------------------------------------------------
NOV56a ------------
NOV56b ------------
NOV56c EVLEKQTTFSQF
NOV56d ------------
NOV56a (SEQ ID NO: 1124)
NOV56b (SEQ ID NO: 1126)
NOV56c (SEQ ID NO: 1128)
NOV56d (SEQ ID NO: 1130)

Further analysis of the NOV56a protein yielded the following properties shown in Table 56C.

TABLE 56C
Protein Sequence Properties NOV56a

| | |
|---|---|
| SignalP analysis: | Cleavage site between residues 41 and 42 |
| PSORT II analysis: | |

| PSG: | a new signal peptide prediction method | |
|---|---|---|
| | N-region: | length 11; pos. chg 0; neg. chg 1 |
| | H-region: | length 1; peak value 0.00 |
| | PSG score: | −4.40 |

| | |
|---|---|
| GvH: | von Heijne's method for signal seq. recognition |
| | GvH score (threshold: −2.1): −2.49 |
| | possible cleavage site: between 38 and 39 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 2 | | |
| | Number of TMS(s) for threshold 0.5: 1 | | |
| | INTEGRAL | Likelihood = | Transmembrane 665-681 |
| | | −13.59 | |

| | |
|---|---|
| PERIPHERAL | Likelihood = 2.44 (at 571) |

| | |
|---|---|
| | ALOM score: −13.59 (number of TMSs: 1) |
| MTOP: | Prediction of membrane topology (Hartmann et al.) |
| | Center position for calculation: 672 |
| | Charge difference: 2.5 C(4.5)-N(2.0) |
| | C > N: C-terminal side will be inside |

>>> membrane topology: type 1b (cytoplasmic tail 665 to 925)

| MITDISC: | discrimination of mitochondrial targeting seq | | | |
|---|---|---|---|---|
| | R content: | 0 | Hyd Moment(75): | 2.75 |
| | Hyd Moment(95): | 4.92 | G content: | 0 |
| | D/E content: | 2 | S/T content: | 2 |

| | |
|---|---|
| | Score: −7.05 |
| Gavel: | prediction of cleavage sites for mitochondrial preseq |
| | cleavage site motif not found |

NUCDISC: discrimination of nuclear localization signals

| | |
|---|---|
| | pat4: KRRK (5) at 690 |
| | pat4: RRKK (5) at 691 |
| | pat7: none |
| | bipartite: none |
| | content of basic residues: 9.2% |
| | NLS Score: 0.15 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: found

| | |
|---|---|
| | LL at 670 |
| LL at 671 | |

checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 89 |
| COIL: | Lupas's algorithm to detect coiled-coil regions |
| | total: 0 residues |

Final Results (k = 9/23):

```
69.6%: nuclear
  8.7%: mitochondrial
  8.7%: cytoplasmic
  4.3%: vacuolar
  4.3%: plasma membrane
  4.3%: vesicles of secretory system
```

>> prediction for CG92715-01 is nuc (k = 23)

A search of the NOV56a protein against the Geneseq database, a proprietary database that contains sequences published in patents and patent publication, yielded several homologous proteins shown in Table 56D.

TABLE 56D
Geneseq Results for NOV56a

| | | NOV56a | Identities/ | | |
|---|---|---|---|---|---|
| | | Residues/ | Similarities for | | |
| Geneseq | Protein/Organism/Length | Match | the Matched | | Expect |
| Identifier | [Patent #, Date] | Residues | Region | | Value |
| ABU12071 | Human NOV16b CG92715-02 protein | 1 . . . 925 | 925/925 | (100%) | 0.0 |
| | SEQ ID 62 - Homo sapiens, 972 aa. | 15 . . . 939 | 925/925 | (100%) | |
| | [WO200281625-A2, 17-OCT-2002] | | | | |
| ABU12070 | Human NOV16a CG92715-01 protein | 1 . . . 925 | 925/925 | (100%) | 0.0 |
| | SEQ ID 60 - Homo sapiens, 925 aa. | 1 . . . 925 | 925/925 | (100%) | |
| | [WO200281625-A2, 17-OCT-2002] | | | | |
| AAU98780 | Human haematopoietic cell line Kg-1a | 1 . . . 925 | 925/925 | (100%) | 0.0 |
| | protein KIAA0918 - Homo sapiens, 958 | 1 . . . 925 | 925/925 | (100%) | |
| | aa. [WO200236804-A2, 10-MAY-2002] | | | | |
| AAB95753 | Human protein sequence SEQ ID | 1 . . . 925 | 925/925 | (100%) | 0.0 |
| | NO: 18665 - Homo sapiens, 958 aa. | 1 . . . 925 | 925/925 | (100%) | |
| | [EP1074617-A2, 07-FEB-2001] | | | | |
| ABB12025 | Human IGFALS homologue, SEQ ID | 1 . . . 925 | 924/925 | (99%) | 0.0 |
| | NO: 2395 - Homo sapiens, 977 aa. | 20 . . . 944 | 924/925 | (99%) | |
| | [WO200157188-A2, 09-AUG-2001] | | | | |

In a BLAST search of public sequence databases, the NOV56a protein was found to have homology to the proteins shown in the BLASTP data in Table 56E.

TABLE 56E
Public BLASTP Results for NOV56a

| | | NOV56a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| O94991 | Hypothetical protein KIAA0918 - | 1 . . . 925 | 925/925 | (100%) | 0.0 |
| | Homo sapiens (Human), 966 aa | 9 . . . 933 | 925/925 | (100%) | |
| | (fragment). | | | | |
| CAD48653 | Sequence 18 from Patent | 22 . . . 925 | 904/904 | (100%) | 0.0 |
| | WO0220569 - Homo sapiens | 1 . . . 904 | 904/904 | (100%) | |
| | (Human), 937 aa. | | | | |
| BAC67208 | Neuronal transmembrane protein | 1 . . . 925 | 895/926 | (96%) | 0.0 |
| | Slitrk5 - Mus musculus (Mouse), | 1 . . . 924 | 908/926 | (97%) | |
| | 957 aa. | | | | |
| Q9H156 | BG115M3.1 (Novel protein) | 46 . . . 925 | 423/886 | (47%) | 0.0 |
| | (SLITL1 protein) - Homo sapiens | 27 . . . 812 | 563/886 | (62%) | |
| | (Human), 845 aa. | | | | |
| Q8NBC7 | Hypothetical protein FLJ33696 - | 46 . . . 781 | 393/742 | (52%) | 0.0 |
| | Homo sapiens (Human), 724 aa | 27 . . . 717 | 511/742 | (67%) | |
| | (fragment). | | | | |

PFam analysis predicts that the NOV56a protein contains the domains shown in the Table 56F.

TABLE 56F
Domain Analysis of NOV56a

| | | | |
|---|---|---|---|
| | | Identities/ | |
| Pfam | NOV56a Match | Similarities for | Expect |
| Domain | Region | the Matched Region | Value |

| | | | | |
|---|---|---|---|---|
| LRR | 154 . . . 177 | 8/25 | (32%) | 0.053 |
| | | 21/25 | (84%) | |
| LRRCT | 235 . . . 285 | 18/54 | (33%) | 2.7e−08 |
| | | 34/54 | (63%) | |
| LRRNT | 373 . . . 406 | 10/36 | (28%) | 0.0099 |
| | | 19/36 | (53%) | |
| LRR | 434 . . . 457 | 8/25 | (32%) | 0.28 |
| | | 17/25 | (68%) | |
| LRR | 458 . . . 481 | 12/25 | (48%) | 0.0022 |
| | | 23/25 | (92%) | |
| LRR | 482 . . . 505 | 5/25 | (20%) | 0.59 |
| | | 19/25 | (76%) | |
| LRR | 506 . . . 529 | 12/25 | (48%) | 0.0077 |
| | | 19/25 | (76%) | |
| LRRCT | 563 . . . 613 | 10/54 | (19%) | 1.9e−05 |
| | | 36/54 | (67%) | |

### Example 57

The NOV57 clone was analyzed, and the nucleotide and encoded polypeptide sequences are shown in Table 57A.

TABLE 57A
NOV57 Sequence Analysis

| | | |
|---|---|---|
| NOV57a, CG92813-01 | SEQ ID NO: 1131 | 15603 bp |
| DNA Sequence | ORF Start: ATG at 518 | ORF Stop: TGA at 15401 |

| CTAATAGAATTCAGCGGCCGCTTTCCCCGGTGCGCAGTTGTGCTTGGACGTTTGTTCCTCCCTCTTCA | | | | |
|---|---|---|---|---|
| CGCTCTTCGCTGCGGGTAAGTTCTAAAGTTTCTGAAGGCCGTTCTTTGCAATGATTCCTCATATACCT | | | | |
| TAGATACAGGCAACTTCTCCCAACTCTCATCCACCCGGGTGAAAACGCTCAGACTATCTGGATTCAAA | | | | |
| AACAAAGTAAAAGGGGGCATATATAAGAGGCTTGAGAAACTTTTCTGGGAACTCAGCTCACAGGAGTG | | | | |
| TCCCGCGGAATGCCCTGCCGCTTTTCGCCACAGCATCTCTCTTGCACTCCGCGTTCAACTGGCTACCT | | | | |
| AGAGTCTTTTGCTGATGCTACTTGCTTTTGCCGGACTGGAGGTTCTTTGAAATAGCAGAGGTCTCAGA | | | | |
| CCAAGCCGTCAGCTGAATCTTTGCTGGCGCTCCTTAATCCCTGTAAATATCATTGCGTTTGCTTCACC | | | | |
| CCTTCCTTCTCTTTATCACATCGTTTTAGGGAGCCAGGACCATGGACTTAGCACCAGACAGGGCTACT | | | | |
| GGCCGCCCGTGGCTCCCGTTGCACACTCTATCAGTATCTCAGCTCCTTCGAGTGTTTTGGCTACTGTC | | | | |
| ATTGCTTCCGGGGCAGGCCTGGGTCCACGGGGCCGAGCCGCGCCAGGTGTTCCAAGTGCTGGAAGAGC | | | | |
| AACCTCCAGGCACTCTGGTAGGCACCATCCAGACGCGCCCCGGCTTCACCTACAGGCTCAGCGAAAGC | | | | |
| CACGCCCTGTTTGCCATAAACAGTAGCACCGGAGCCCTGTACACCACCTCCACCATCGACCGCGAGAG | | | | |
| CCTGCCCAGCGACGTGATCAACCTGGTGGTCCTTTCCAGCGCGCCCACCTACCCCACCGAAGTGCGAG | | | | |
| TGCTGGTGCGGGACCTCAATGACAACGCCCCCGTTTTCCCGGACCCCTCTATCGTGGTCACTTTCAAG | | | | |
| GAAGACAGTAGCAGCGGACGCCAAGTCATCTTAGACACCGCCACCGACTCGGACATCGGCTCAAACGG | | | | |
| TGTGGACCACCGCTCCTACCGCATCATCCGCGGCAATGAGGCGGGGCGCTTCCGTCTGGACATCAACC | | | | |
| TGAACCCGAGCGGCGAGGGAGCGTTCCTGCATCTGGTGTCCAAGGGCGGACTGGACCGTGAGGTCACT | | | | |
| CCGCAGTACCAGCTCCTGGTTGAGGTGGAGGACAAGGGTGAGCCTAAGCGGCGGGGCTACCTTCAGGT | | | | |
| AAACGTGACTGTGCAAGACATTAATGACAACCCCCCGGTTTTTGGCAGTTCTCACTACCAGGCGGGGG | | | | |
| TGCCTGAGGACGCGGTTGTGGGTTCCAGCGTCCTCCAGGTGGCGGCGGCGGACGCGGACGAGGGCACC | | | | |
| AACGCGGACATCCGCTATCGCCTGCAGGACGAGGGGACCCCCTTCCAAATGGACCCTGAGACGGGACT | | | | |
| TATCACGGTGCGGGAGCCCCTGGACTTCGAAGCTCGGCGCCAATACTCGCTTACGGTGCAGGCGATGG | | | | |
| ACAGAGGCGTGCCTTCCCTCACTGGGCGCGCCGAGGCGCTGATTCAGCTGCTGGACGTGAATGACAAT | | | | |
| GACCCGGTAGTGAAGTTCCGCTACTTCCCGGCCACCTCGOGCTACGCCTCGGTAGATGAGAATGCTCA | | | | |
| AGTGGGCACCGTGGTGGCTCTGCTCACCGTGACGGACGCAGATTCTCCCGCGGCCAACGGGAACATCT | | | | |
| CCGTGCAAATTCTCGGGGGCAATGAGCAGCGCCACTTTGAAGTGCAAAGCAGCAAAGTGCCGAACCTG | | | | |
| AGCCTAATCAAGGTGGCCAGCGCCTTGGACCGCGAGCGCATCCCTTCCTACAACCTCACAGTTTCCGT | | | | |
| CTCTGATAACTACGGGGCGCCCCCTGGCGCAGCAGTCCAGGCGCGCTCTTCTGTGGCAAGCCTGGTGA | | | | |
| TTTTTGTTAATGACATCAATGACCATCCTCCTGTCTTTTCACAGCAAGTGTACAGAGTGAACCTGAGC | | | | |
| GAGGAGGCGCCTCCGGGAAGCTATGTGAGTGGGATATCTGCCACTGATGGCGACTCTGGTCTCAATGC | | | | |
| TAATCTGCGTTACAGCATTGTCTCTGGCAATGGACTGGGATGGTTCCATATCAGTGAACATAGCGGCC | | | | |
| TCGTGACCACTGGGTCCTCTGGGGGCCTGGACCGTGAACTTGCTTCCCAGATTGTTCTGAATATAAGT | | | | |
| GCCCGGGACCAGGGAGTTCACCCCAAGGTGTCCTATGCCCAGCTTGTAGTAACTCTCCTAGATGTGAA | | | | |
| TGATGAAAAGCCAGTATTTAGCCAGCCAGAAGGGTATGATGTGTCTGTGGTTGAGAATGCCCCAACAG | | | | |
| GGACAGAACTGTTGATGCTCAGGGCAACTGACGGGGACCTGGGTGACAACGGAACAGTGCGCTTCTCC | | | | |
| TTACAAGAGGCAGAGACTGACCGGAGGTCCTTCCGTCTGGATCCTGTGTCTGGGAGGTTGAGTACTAT | | | | |
| TTCCTCCTTGGACAGAGAAGAGCAAGCCTTCTACTCCCTGTTGGTTCTGGCCACAGATCTGGGCTCCC | | | | |
| CTCCCCAGTCATCAATGGCTCGCATAAATGTGAGTCTTCTGGATATAAATGATAACAGCCCTGTCTTC | | | | |
| TACCCGGTCCAATACTTTGCTCACATTAAGGAGAATGAGCCTGGAGGTAGCTACATCACCACTGTGTC | | | | |
| TGCCACTGACCCAGACTTGGGTACCAATGGTACTGTCAAATATAGCATATCTGCTGGGGACAGGTCTC | | | | |
| GGTTTCAGGTCAATGCTCAGAGTGGGGTTATTTCTACAAGAATGGCCCTAGACAGAGAAGAAAAAACA | | | | |
| GCTTATCAGTTGCAAATAGTAGCTACTGATGGTGGCAATTTACAATCTCCCAACCAGGCAATAGTAAC | | | | |
| CATCACTGTATTGGACACTCAAGACAACCCACCTGTATTCAGTCAGGTTGCCTACAGCTTTGTGGTTT | | | | |
| TTGAGAACGTGGCGCTGGGATATCATGTGGGTAGTGTGTCTGCATCCACCATGGATCTCAATTCCAAC | | | | |
| ATCAGTTATCTCATTACTACTGGGGATCAGAAAGGTATGTTTGCTATCAACCAGGTCACTGGGCAGCT | | | | |
| TACCACAGCAAATGTGATTGATAGAGAAGAGCAATCCTTTTATCAGCTGAAGGTAGTGGCCAGTGGGG | | | | |
| GCACAGTGACTGGAGACACTATGGTTAACATAACAGTTAAGGATTTGAATGACAACTCTCCCCATTTC | | | | |
| CTTCAGGCAATAGAGAGTGTAAATGTGGTGGAGAATTGGCAGGCAGGTCACAGCATTTTCCAGGCCAA | | | | |
| AGCTGTGGACCCTGATGAAGGTGTCAATGGCATGGTACTCTATAGTCTGAAGCAAAACCCCAAGAACC | | | | |
| TGTTTGCTATCAATGAAAAGAATGGCACTATTAGTCTGCTTGGGCCCCTGGATGTTCATGCTGGCTCC | | | | |
| TACCAAATAGAGATCTTGGCATCTGACATGGGTGTCCCACAGCTCTCCTCTAGTGTCATCCTAACAGT | | | | |
| TTATGTCCATGATGTAAATGACAATTCACCAGTGTTTGACCAACTCTCTTATGAAGTCACCCTTTCTG | | | | |
| AGTCAGAACCTGTGAATTCTCGATTCTTTAAAGTACAAGCTTCTGATAAGGATTCAGGAGCAAATGAT | | | | |
| GGTCAATTGTATATAAAAAGTGAACTGGACCGTGAACTTCAAGACAGATATGTTTTAATGGTTGTTGC | | | | |
| TTCTGACAGAGCAGTGGAACCCCTTAGTGCTACTGTGAATGTTACTGTAATTTTAGAAGATGTAAATG | | | | |
| ATAACAGACCTCTTTTTAACAGTACCAATTACACATTTTACTTCGAAGAAGAGCAGAGGGCTGGGTCG | | | | |
| TTTGTGGGCAAAGTAAGTGCTGTAGATAAAGACTTTGGGCCAAATGGAGAAGTAAGGTATTCTTTTGA | | | | |
| AATGGTGCAGCCAGATTTTGAGTTGCATGCCATCAGTGGGGAAATTACAAATACTCATCAGTTTGACA | | | | |
| GGGAGTCTCTTATGAGGCGGAGAGGGACTGCTGTGTTTAGCTTTACAGTCATAGCAACAGATCAGGGG | | | | |
| ATCCCTCAGCCTCTCAAGGATCAGGCCACTGTACATGTTTACATGAAGGATATAAATGATAATGCTCC | | | | |
| CAAATTTTTAAAAGACTTTTACCAAGCTACAATATCAGAATCAGCAGCCAATCTGACACAAGTGTTAA | | | | |
| GAGTATCTGCCTCAGATGTTGATGAAGGTAATAATGGACTTATTCACTATTCTATAATAAAAGGAAAT | | | | |
| GAAGAAAGACAGTTTGCTATAGACAGTACCTCTGGTCAGGTAACACTAATTGGCAAATTAGACTATGA | | | | |
| AGCAACACCTGCCTATTCCCTTGTAATTCAAGCAGTGGATTCAGGGACAATCCCCCTCAATTCAACGT | | | | |
| GTACTTTAAATATTGATATTTTAGATGAAAATGACAATACCCCTTCTTTCCTTAAATCAACACTGTTT | | | | |
| GTTGATGTTTTGGAAAACATGAGAATTGGTGAACTCGTGTCCTCTGTTACTGCAACTGATTCCGATTC | | | | |
| AGGTGACAATGTTGATTTATATTACAGTATTACTGGGACTAACAACCACGGAACTTTTAGCATTAGCC | | | | |
| CAAACACTGGGAGTATTTTTCTTGCCAAAAAACTGGACTTTGAAACACAGTCTTTGTATAAATTAAAT | | | | |
| ATAACAGCAAAAGACCAAGGAAGACCTCCTCGTTCATCTACAATGTCAGTGGTTATTCACGTGAGGGA | | | | |
| CTTTAATGACAATCCTCCTAGCTTTCCTCCTGGAGATATTTTCAAGTCTATTGTTGAGAACATTCCCA | | | | |
| TTGGTACATCTGTCATTTCAGTGACTGCACATGACCCTGATGCAGACATTAATGGTCAACTATCCTAC | | | | |
| ACAATCATTCAACAGATGCCAAGAGGCAACCACTTTACCATAGATGAAGTCAAAGGGACTATATATAC | | | | |
| TAATGCTGAAATAGATCGGGAATTTGCTAATCTCTTTGAGTTGACTGTAAAAGCCAATGATCAAGCTG | | | | |
| TGCCAATAGAAACTAGACGGTATGCTTTGAAGAACGTGACCATTTTGGTTACAGACCTCAATGACAAT | | | | |
| GTCCCAATGTTTATATCACAAAACGCCCTTGCTGCAGACCCATCAGCTGTGATTGGTTCCGTTCTGAC | | | | |
| AACAATTATGGCTGCTGACCCAGATGAAGGTGCTAATGGAGAAATAGAGTATGAGATCATCAATGGGG | | | | |
| ACACAGACACCTTCATTGTTGATCGTTATAGTGGAGACCTGAGAGTGGCTTCAGCGTTGGTGCCTTCA | | | | |
| CAGTTGATCTACAATCTCATAGTTTCAGCAACAGACCTTGGGCCTGAAAGGAGGAAATCGACCACTGA | | | | |
| ATTGACCATCATTCTTCAGGGCCTTGATGGACCTGTTTTTACTCAACCCAAATATATAACTATTTTGA | | | | |
| AGGAAGGAGAACCCATTGGCACAAACGTGATATCAATAGAAGCAGCTAGCCCCAGAGGATCTGAGGCC | | | | |
| CCAGTGGAGTATTATATTGTTTCAGTTCGTTGTGAAGAAAAAACTGTTGGACGCCTCTTTACTATTGG | | | | |
| ACGACATACTGGTATAATTCAGACCGCAGCCATTCTGGACCGGGAGCAAGGAGCATGTCTTTACCTGG | | | | |
| TGGATGTTTATGCCATAGAAAAATCAACTGCTTTTCCCAGAACACAGAGAGCAGAGGTAGAAACAACA | | | | |
| CTTCAGGATATCAATGACAATCCACCAGTATTTCCAACGGACATGCTGGATCTCACGGTAGAGGAGAA | | | | |
| CATTGGAGATGGCTCTAAGATTATGCAGCTGACAGCCATGGATGCTGACGAGGTGCAAATGCTCTCGT | | | | |
| CACATACACTATCATTAGTGGGTTCTTTGGTAGCAGCCATTTTAGCCACGGATGATGACTCTGGTGTG | | | | |
| AATGGAGAAATTACATATATTGTGAATGAAGATGATGAAGATGGCATCTTTTTCCTGAATCCTATTAC | | | | |
| TGGGGTCTTTAATTTGACTCGATTATTAGATTATGAAGTACAGCAATATTATATCCTCACTGTTCGAG | | | | |
| CAGAAGATGGTGGGGGACAATTTACTACCATCAGAGTTTATTTCAATATTCTAGATGTAAATGATAAT | | | | |
| CCACCTATTTTCAGCTTGAATTCATACAGCACATCTTTAATGGAGAATCTACCTGTGGGATCTACTGT | | | | |
| TCTTGTGTTTAATGTTACTGATGCAGATATGATGAAGGCAGAAATAAAGATGTTCTTTGAAACCAGTG | | | | |
| AGAACAAAGACACAACATACCAGAATCTCTGGGACACATTCAAAGCAGTGTGTAGAGGGAAATTTATA | | | | |
| GCACTAAATGCCCACAAGAGAAAGCAGGAAAGATCCAAAATTGACACCCTAACATCACAATTAAAAGA | | | | |
| ACTAGAAAAGCAAGAGCAAACACATTCAAAAGCTAGCAGAAGGCAAGAAATAACTAAAATCAGAGCAG | | | | |
| AACTGAAGGATATAGAGACACAAAAAACCCTTCAAAAAATTAATGAATCCAGGAGCTGGTTTTTTGAA | | | | |
| AGGATCAACAAAATTGATAGACCGCTAGCAAGACTAATAAAGAAGAAAACAGAGAAGAATCAAATAGA | | | | |
| CGCAATAAAAAATGATAAAGGGGATATCACCATCGATCCCACAGAAATACAAACTACCATCAGAGAAT | | | | |
| ACTGCAAACACCTCTATGCAAATAAACTAGAAAATCTAGAAGAAATGGATAAATTCCTCGACACATAC | | | | |
| ACCCTCCCAAGACTAAACCAGGAAGAAGTTGAATCTCTGAATAGACCAATAACAGACTCTGAAACTGT | | | | |
| GGCAATAATCAATAGCTTACCAACCAAAAAGAGTCCAGGACCAGATGGATTCACAGCCGAATTCTACC | | | | |
| AGATGATAACAACCCCAGTCTTTGCACAAGCTTTGTATAAAGTGGAGATTAATGAAAACACACTTACT | | | | |
| GGAACAGATATAATACAAGTGTTCGCAGCAGATGGAGATGAAGGCACAAATGGACAGGTTCGCTATGG | | | | |
| CATTGTTAATGGTAATACCAATCAGGAATTTCGGATAGACTCTGTCACAGGTGCCATCACTGTCGCTA | | | | |
| AACCTTTGGATAGAGAAAAGACCCCTACCTACCATTTAACTGTTCAGGCAACAGATCGAGGCAGCACA | | | | |
| CCCAGAACTGATACCTCCACGGTCAGCATTGTTCTACTGGATATTAATGACTTTGTTCCTGTATTTGA | | | | |
| GCTATCTCCATATTCTGTAAATGTCCCTGAGAATTTAGGGACACTACCCAGAACAATTCTTCAGACTG | | | | |
| CTTCGCCTTGCGTGAGGTTTGCCAGCGCCAGTAAAGCGTATTTCACAACAATTCCTGAGGATGCACCA | | | | |
| ACTGGAACAGATGTTTTATTGGTAAATGCCTCAGATGCTGATGCTTCAAAGAATGCAGTTATAAGTTA | | | | |
| TAGGATCATCGGTGGAAACTCTCAGTTCACGATCAACCCATCGACAGGACAAATCATCACCAGCGCAT | | | | |
| TGTTAGATAGGGAAACAAAAGATAATTATACTTTGGTAGTGGTCTGCAGTGATGCGGGATCCCCAGAG | | | | |
| CCTCTTTCCAGTTCCACCAGTGTGCTTGTCACTGTGACTGATGTCCATGACAATCCACCAAGATTTCA | | | | |
| GCATCACCCATATGTCACTCACATCCCATCTCCTACTCTTCCAGGTTCCTTTGTCTTTGCGGTTACAG | | | | |
| TCACAGATGCTGATATTGGACCAAATTCTGAACTGCATTATTCTCTTTCGGGTAGAAATTCTGAAAAA | | | | |
| TTTCACATTGACCCACTGAGGGGAGCCATTATGGCCGCCGGACCACTAAACGGAGCTTCAGAAGTGAC | | | | |
| ATTTTCTGTGCATGTAAAAGATGGTGGCTCATTTCCAAAGACAGATTCTACAACAGTGACTGTTAGAT | | | | |
| TCGTGAATAAGGCCGATTTCCCTAAAGTCAGAGCCAAAGAACAAACGTTCATGTTTCCTGAAAACCAA | | | | |
| CCAGTCAGCTCTCTTGTCACCACCATCACAGGATCCTCTTTAAGAGGAGAACCTATGTCATATTATAT | | | | |
| CGCAAGTGGGAATCTTGGCAATACTTTCCAGATTGATCAGTTAACAGGGCAGGTGTCTATTAGTCAAC | | | | |
| CTCTGGATTTTGAAAAGATACAAAAATATGTTGTATGGATAGAGGCCAGAGACGGTGGTTTCCCTCCT | | | | |
| TTCTCCTCTTACGAGAAACTTGATATAACAGTATTAGATGTCAATGATAATGCCCCAATTTTTAAGGA | | | | |
| AGACCCATTTATATCTGAAATATTGGAAAACCTTTCCCCTCGAAAAATACTTACTGTTTCGGCAATGG | | | | |
| ACAAGGACAGTGGACCCAATGGACAGTTAGATTATGAAATTGTTAATGGCAACATGGAAAATAGTTTC | | | | |
| AGTATCAATCATGCTACTGGTGAAATTAGAAGCGTTAGACCTTTGGACAGGGAAAAAGTATCTCATTA | | | | |
| TGTCCTAACCATAAAATCATCAGACAAAGGGTCCCCGTCTCAGAGTACTTCAGTAAAAGTCATGATTA | | | | |
| ACATTTTAGATGAAAATGATAATGCCCCTAGGTTTTCTCAGATATTTAGTGCCCATGTTCCTGAAAAT | | | | |
| TCCCCCTTAGGATACACAGTTACCCGTGTCACAACTTCTGATGAAGACATTGGGATCAATGCAATTAG | | | | |
| TAGATATTCTATAATGGATGCAAGTCTTCCATTTACAATTAATCCCAGCACAGGGGATATTGTCATAA | | | | |
| GCAGACCTTTAAATAGGGAAGATACAGACCGTTACAGAATTCGAGTTTCCGCACATGATTCTGGGTGG | | | | |
| ACTGTAAGTACAGATGTCACAATATTTGTGACAGACATCAATGACAATGCTCCAAGATTTAGCAGAAC | | | | |
| TTCCTATTATTTAGATTGCCCTGAACTTACTGAGATTGGCTCCAAAGTAACTCAGGTATTTGCAACAG | | | | |
| ATCCTGATGAGGGATCAAATGGACAAGTGTTTTATTTCATAAAATCCCAATCAGAATATTTCAGGATT | | | | |
| AATGCCACCACTGGAGAGATTTTCAATAAACAGATCTTAAAATACCAAAATGTCACTGGCTTCAGTAA | | | | |
| TGTGAATATCAACAGGCATAGTTTTATAGTGACATCTTCAGATCGAGGTAAACCTTCCTTAATTAGTG | | | | |
| AGACAACAGTTACCATCAATATAGTGGACAGTAATGACAATGCACCTCAATTTCTTAAAAGTAAATAT | | | | |
| TTCACTCCAGTCACCAAAAATGTTAAGGTTGGTACGAAGTTAATCAGAGTTACAGCAATAGATGACAA | | | | |
| AGATTTTGGACTGAATTCAGAAGTGGAGTATTTCATTTCTAATGATAACCATTTAGGAAAATTTAAGT | | | | |
| TGGACAATGATACGGGGTGGATTTCAGTAGCATCCTCCCTGATTTCTGACTTGAACCAAAACTTTTTT | | | | |
| ATCACAGTCACTGCAAAGGATAAGGGAAACCCTCCACTTTCTTCCCAAGCAACTGTTCACATAACTGT | | | | |
| CACTGAGGAAAACTACCATACACCTGAATTCTCTCAAAGCCACATGAGTGCAACCATCCCTGAGAGCC | | | | |
| ATAGCATTGGGTCCATTGTCAGAACTGTTTCTGCAAGAGATAGAGATGCAGCGATGAATGGCTTGATT | | | | |
| AAGTACAGCATTTCTTCAGGAAATGAAGAAGGCATTTTTGCAATCAATTCTTCTACAGGTATATTAAC | | | | |
| ACTAGCCAAAGCTCTTGATTATGAGCTATGCCAGAAACACGAAATGACGATTAGTGCTATAGATGGAG | | | | |
| GATGGGTTGCAAGAACTGGTTACTGCAGTGTGACCGTAAATGTGATTGATGTGAATGATAATTCTCCA | | | | |
| GTATTCCTCTCTGATGACTATTTCCCTACTGTTTTGGAAAATGCCCCAAGTGGAACAACAGTTATCCA | | | | |
| CCTAAATGCAACAGATGCTGACTCTGGAACAAATGCTGTGATTGCGTATACTGTACAGTCATCTGACA | | | | |
| GTGACCTCTTTGTCATTGACCCTAACACAGGAGTCATAACCACTCAAGGCTTCTTGGATTTTGAAACC | | | | |
| AAGCAGAGCTACCATCTTACTGTGAAAGCCTTCAATGTCCCCGATGAGGAAAGGTGTAGCTTTGCCAC | | | | |
| TGTTAATATACAATTAAAAGGGACAAATGAATATGTGCCCCGTTTTGTTTCCAAACTTTACTATTTTG | | | | |
| AAATCTCAGAAGCAGCTCCTAAAGGTACTATTGTTGGAGAAGTGTTTGCTAGCGACCGTGATTTGGGC | | | | |
| ACTGATGGGGAGGTACACTATTTGATTTTTGGTAATAGTCGAAAGAAGGGTTTCCAGATCAATAAGAA | | | | |
| GACTGGACAGATTTATGTTTCTGGAATTCTTGATCGAAAAAAAGAAGAAAGGGTGTCTTTGAAGGTAT | | | | |
| TGGCCAAGAACTTTGGCAGCATTAGAGGTGCAGATATAGATGAGGTCACTGTAAATGTCACCGTGCTT | | | | |
| GATGCAAATGACCCACCCATTTTTACTCTAAACATCTACAGTGTGCAGATCAGTGAAGGGGTCCCAAT | | | | |
| AGGAACTCATGTGACCTTTGTCAGTGCCTTTGACTCAGACTCCATCCCCAGCTGGAGCAGGTTTTCTT | | | | |
| ACTTCATCGGATCAGGGAATGAAAATGGTGCCTTTTCTATTAATCCGCAGACAGGACAGATCACCGTT | | | | |
| ACTGCAGAATTAGATCGAGAAACCCTTCCCATCTATAATCTCTCAGTTTTGGCTGTTGATTCAGGGAC | | | | |
| CCCCTCAGCTACAGGTAGTGCCTCTTTATTAGTCACCCTGGAAGATATAAATGATAACGGGCCCATGC | | | | |
| TGACTGTCAGTGAAGGAGAAGTCATGGAAAACAAACGGCCAGGCACTTTGGTGATGACCCTTCAGTCC | | | | |
| ACTGACCCTGATCTCCCTCCAAATCAAGGTCCCTTTACTTATTACTTGCTGAGCACAGGTCCTGCCAC | | | | |
| CAGTTATTTCAGTCTGAGCACTGCTGGAGTTCTGAGCACAACCAGAGAGATTGACAGAGAGCAGATTG | | | | |
| CAGACTTCTATCTGTCTGTGGTTACCAAGGATTCTGGTGTTCCTCAAATGTCTTCCACAGGAACTGTG | | | | |
| CATATCACAGTTATAGACCAAAATGACAATCCTTCACAGTCTCGGACGGTGGAGATATTTGTTAATTA | | | | |
| TTATGGTAACTTGTTTCCCGGTGGGATTTTAGGCTCTGTGAAGCCACAGGATCCAGATGTGTTAGACA | | | | |
| GCTTCCACTGCTCCCTTACTTCAGGAGTTACCAGCCTCTTCAGTATTCCAGGGGGTACTTGTGATCTG | | | | |
| AATTCCCAGCCAAGGTCCACAGATGGCACGTTTGATCTGACTGTCCTTAGCAATGATGGAGTTCACAG | | | | |
| CACAGTCACGAGCAACATCCGAGTTTTCTTTGCTGGATTTTCCAATGCCACAGTGGATAACAGCATCT | | | | |
| TACTTCGTCTCGGCGTACCAACAGTAAAGGACTTCTTGACCAACCACTATCTTCATTTTTTACGCATT | | | | |
| GCCAGCTCACAGCTGACAGGCTTAGGGACTGCTGTGCAACTGTACAGTGCATATGAAGAGAACAATAG | | | | |
| AACGTTTCTTTTGGCAGCTGTGAAGCGAAATCATAATCAGTATGTGAATCCCAGTGGCGTAGCCACCT | | | | |
| TCTTTGAAAGCATCAAAGAGATCCTTCTCCGGCAGAGTGGAGTAAAGGTGGAATCTGTGGATCATGAC | | | | |
| TCCTGTGTGCATGGCCCATGTCAGAATGGAGGGAGCTGTCTACGAAGATTGGCTGTGAGCTCCGTATT | | | | |
| AAAAAGCCGTGAGAGTCTTCCAGTCATCATCGTGGCAAATGAACCTCTGCAGCCTTTCTTATGCAAGT | | | | |
| GTCTGCCAGGATATGCGGGTAGCTGGTGTGAAATAGATATAGATGAATGTCTTCCATCACCTTGCCAC | | | | |
| AGTGGTGGAACCTGTCACAATTTAGTGGGAGGATTTTCATGCAGCTGCCCAGATGGCTTCACTGGTAG | | | | |
| GGCGTGTGAGAGAGATATCAATGAGTGCCTGCAGAGTCCTTGCAAGAATGGTGCCATCTGCCAGAATT | | | | |
| TTCCAGGAAGCTTCAACTGTGTTTGCAAAACTGGATACACAGGTATGACAACGTTTGTACTTTTCTCA | | | | |
| CTAAGACTTGGAAAATGTGTGAATCTTCAGTCAATTACTGTGAATGCAACCCCTGCTTTAATGGTGGT | | | | |
| TCCTGCCAAAGTGGTGTGGATTCTTATTATTGTCATTGTCCATTTGGTGTCTTTGGAACACTGCGAGT | | | | |
| TGAACAGTTATGGATTTGAGGAGTTATCATACATGGAATTTCCAAGCTTGGACCCCAATAACAACTAT | | | | |
| ATTTATGTCAAATTTGCCACGATTAAAAGTCATGCCTTATTGCTTTACAACTATGACAACCAGACAGG | | | | |
| CGACCGGGCTGAGTTTTTGGCCCTTGAAATTGCCGAAGAAAGACTAAGATTCTCTTATAATTTAGGCA | | | | |
| GTGGTACATATAAGCTCACCACCATGAAGAAGGTGTCAGATGGACATTTTCACACTGTGATTGCCAGG | | | | |
| AGAGCAGGAATGGCAGCCTCCTTAACTGTGGACTCCTGTTCTGAGAACCAAGAGCCAGGATATTGTAC | | | | |
| TGTCAGTAATGTGGCAGTTTCAGATGACTGGACTCTTGATGTTCAGCCAAATAGAGTTACAGTTGGAG | | | | |
| GTATCAGATCTCTAGAACCAATCCTTCAGAGAAGAGGACACGTGGAAAGCCATGATTTTGTTGGGTGT | | | | |
| ATAATGGAGTTTGCAGTCAATGGAAGGCCTCTGGAACCCAGCCAAGCTTTGGCAGCACAAGGCATCCT | | | | |
| AGATCAGTATGGCGATTTTATTTCTTACTGTTTTAAAGAAAAAAAATGCAAAAAAGTATGCTTCACTG | | | | |
| TTACTCCTGACACTGCCTTATCATTAGAAGGCAAAGGGCGCTTGGACTACCACATGAGTCAGAATGAG | | | | |
| AAGCGGGAATATTTGTTAAGGCAAAGCTTACGAGGTGCCATGTTGGAGCCTTTTGGTGTGAACAGTCT | | | | |
| GGAAGTAAAATTTAGGACCAGAAGCGAGAATGGCGTTTTAATCCATATCCAAGAAAGCAGCAATTACA | | | | |
| CTACTGTGAAGGGAATGTGTGAATCTTCAGTCAATTACTGTGAATGCAACCCCTGCTTTAATGGTGGT | | | | |
| TCCTGCCAAAGTGGTGTGGATTCTTATTATTGTCATTGTCCATTTGGTGTCTTTGGAAAACACTGCGA | | | | |
| GTTGAACAGTTATGGATTTGAGGAGTTATCATACATGGAATTTCCAAGCTTGGACCCCAATAACAACT | | | | |
| ATATTTATGTCAAATTTGCCACGATTAAAAGTCATGCCTTATTGCTTTACAACTATGACAACCAGACA | | | | |
| GGCGACCGGGCTGAGTTTTTGGCCCTTGAAATTGCCGAAGAAAGACTAAGATTCTCTTATAATTTAGG | | | | |
| CAGTGGTACATATAAGCTCACCACCATGAAGAAGGTGTCAGATGGACATTTTCACACTGTGATTGCCA | | | | |
| GGAGAGCAGGAATGACTCTTGATGTTCAGCCAAATAGAGTTACAGTTGGAGGTATCAGATCTCTAGAA | | | | |
| CCAATCCTTCAGAGAAGAGGACACGTGGAAAGCCATGATTTTGTTGGGTGTATAATGGAGTTTGCAGT | | | | |
| CAATGGAAGGCCTCTGGAACCCAGCCAAGCTTTGGCAGCACAAGGCATCCTAGATCAGTATGGCGATT | | | | |
| TTATTTCTTACTGTTTTAAAGAAAAAAAATGCAAAAAGTATGCTTCACTTGGCCTCCATCTCGGGAAG | | | | |
| CATAGCTTGGCCTCCATCTCAAAAACAGATCCCTCAGTGAAGATTGGCTGCCGTGGCCCGAACATTTG | | | | |
| TGCCAGCAACCCCTGCTGGGGTGATTTGCTGTGCATTAATCAGTGGTATGCCTACAGGTGTGTCCCTC | | | | |
| CTGGGGACTGTGCCTCCCACCCGTGCCAGAATGGTGGCAGCTGTGAGCCAGGCCTGCACTCCGGCTTC | | | | |
| ACCTGTAGCTGCCCAGACTCGCACACGGGAAGGACCTGTGAGATGGTGGTGGCCTGTCTTGGCGTCCT | | | | |
| CTGTCCTCAGGGGAAGGTGTGCAAAGCTGGAAGTCCTGCGGGGCATGTCTGTGTTCTGAGTCAGGGCC | | | | |
| CTGAAGAGATCTCTCTGCCTTTGTGGGCTGTGCCTGCCATCGTGGGCAGCTGCGCAACCGTCTTGGCC | | | | |
| CTCCTGGTCCTTAGCCTGATCCTGTGTAACCAGTGCAGGGGGAAGAAGGCCAAAAATCCCAAAGAGGA | | | | |
| GAAGAAACCGAAGGAGAAGAAGAAAAAGGGAAGTGAGAACGTTGCTTTTGATGACCCTGACAATATCC | | | | |
| CTCCCTATGGGGATGACATGACTGTGAGGAAGCAGCCTGAAGGGAACCCAAAACCAGATATCATTGAA | | | | |
| AGGGAAAACCCCTACCTTATCTATGATGAAACTGATATTCCTCACAACTCAGAAACCATCCCCAGCGC | | | | |
| CCCTTTGGCATCTCCAGAGCAGGAGATAGAGCACTATGACATTGACAACGCCAGCAGCATCGCCCCTT | | | | |
| CGGATGCAGACATCATTCAACACTACAAGCAGTTCCGCAGCCACACACCAAAATTTTCAATCCAGAGG | | | | |
| CACAGTCCCCTAGGCTTTGCAAGGCAATCCCCCATGCCCTTAGGAGCAAGCAGTTTGACTTACCAGCC | | | | |
| TTCATATGGTCAAGGTTTGAGAACCAGCTCCCTAAGCCACTCAGCATGCCCAACTCCCAACCCTCTGT | | | | |
| CTCGACACAGTCCAGCCCCTTTCTCCAAATCTTCTACGTTCTATAGAAACAGCCCAGCAAGGGAATTG | | | | |
| CATCTTCCTATAAGGGATGGTAATACTTTGGAAATGCATGGTGACACCTGCCAACCTGGCATTTTCAA | | | | |
| CTATGCCACAAGGCTGGGAAGGAGAAGCAAGAGTCCTCAGGCCATGGCATCACATGGTTCTAGACCAG | | | | |
| GGAGTCGCCTAAAGCAGCCGATTGGGCAGATTCCACTGGAATCTTCTCCTCCAGTCGGACTTTCTATT | | | | |
| GAAGAAGTGGAGAGGCTCAACACACCTCGCCCTAGAAACCCAAGTATCTGCAGTGCAGACCATGGGAG | | | | |
| GTCTTCTTCAGAGGAGGACTGCAGAAGGCCACTGTCTAGAACAAGGAATCCAGCGGATGGCATTCCAG | | | | |
| CTCCAGAATCCTCTTCTGATAGTGACTCCCATGAATCTTTCACTTGCTCAGAAATGGAATATGACAGG | | | | |
| GAGAAGCCAATGGTATATACTTCCAGAATGCCCAAATTATCTCAAGTCAATGAATCTGATGCAGATGA | | | | |
| TGAAGATAATTATGGAGCCAGACTGAAGCCTCGAAGGTACCACGGTCGCAGGGCCGAGGGAGGACCTG | | | | |
| TGGGCACCCAGGCAGCAGCACCAGGCACTGCTGACAACACACTGCCCATGAAGCTAGGGCAGCAAGCA | | | | |
| GGGACTTTCAACTGGGACAACCTTTTGAACTGGGGCCCTGGCTTTGGCCATTATGTAGATGTTTTTAA | | | | |
| AGATTTGGCATCTCTTCCAGAAAAAGCAGCAGCAAATGAAGAAGGCAAAGCTGGGACAACTAAACCAG | | | | |
| TCCCCAAAGATGGGGAAGCAGAACAGTATGTGTGAAGTTTATGTACTGGCACTATAAAATATAAAAAC | | | | |
| AAGAAATAATACTCAAACCATTGTAAAGTTGCTGACTAGGTTGGGTCACATTTGAAAAACAGGCCAGT | | | | |
| ATGGACTAGTGGTGGAGGGAAAACTTTAAAAATAATAACCACAATGCTGCTGAAACAGACTCACAACA | | | | |
| ACTCTTAATTTAAACATGTGTGGTTGAATTC | | | | |
| | NOV57a, CG92813-01 | SEQ ID NO: 1132 | 4961 aa | MW at 543673.9kD |
| | Protein Sequence | | | |

MDLAPDRATGRPWLPLHTLSVSQLLRVFWLLSLLPGQAWVHGAEPRQVFQVLEEQPPGTLVGTIQTRP
GFTYRLSESHALFAINSSTGALYTTSTIDRESLPSDVINLVVLSSAPTYPTEVRVLVRDLNDNAPVFP
DPSIVVTFKEDSSSGRQVILDTATDSDIGSNGVDHRSYRIIRGNEAGRFRLDINLNPSGEGAFLHLVS
KGGLDREVTPQYQLLVEVEDKGEPKRRGYLQVNVTVQDINDNPPVFGSSHYQAGVPEDAVVGSSVLQV
AAADADEGTNADIRYRLQDEGTPFQMDPETGLITVREPLDFEARRQYSLTVQAMDRGVPSLTGRAEAL
IQLLDVNDNDPVVKFRYFPATSRYASVDENAQVGTVVALLTVTDADSPAANGNISVQILGGNEQRHFE
VQSSKVPNLSLIKVASALDRERIPSYNLTVSVSDNYGAPPGAAVQARSSVASLVIFVNDINDHPPVFS
QQVYRVNLSEEAPPGSYVSGISATDGDSGLNANLRYSIVSGNGLGWFHISEHSGLVTTGSSGGLDREL
ASQIVLNISARDQGVHPKVSYAQLVVTLLDVNDEKPVFSQPEGYDVSVVENAPTGTELLMLRATDGDL
GDNGTVRFSLQEAETDRRSFRLDPVSGRLSTISSLDREEQAFYSLLVLATDLGSPPQSSMARINVSLL
DINDNSPVFYPVQYFAHIKENEPGGSYITTVSATDPDLGTNGTVKYSISAGDRSRFQVNAQSGVISTR
MALDREEKTAYQLQIVATDGGNLQSPNQAIVTITVLDTQDNPPVFSQVAYSFVVFENVALGYHVGSVS
ASTMDLNSNISYLITTGDQKGMFAINQVTGQLTTANVIDREEQSFYQLKVVASGGTVTGDTMVNITVK
DLNDNSPHFLQAIESVNVVENWQAGHSIFQAKAVDPDEGVNGMVLYSLKQNPKNLFAINEKNGTISLL
GPLDVHAGSYQIEILASDMGVPQLSSSVILTVYVHDVNDNSPVFDQLSYEVTLSESEPVNSRFFKVQA
SDKDSGANDGQLYIKSELDRELQDRYVLMVVASDRAVEPLSATVNVTVILEDVNDNRPLFNSTNYTFY
FEEEQRAGSFVGKVSAVDKDFGPNGEVRYSFEMVQPDFELHAISGEITNTHQFDRESLMRRRGTAVFS
FTVIATDQGIPQPLKDQATVHVYMKDINDNAPKFLKDFYQATISESAANLTQVLRVSASDVDEGNNGL
IHYSIIKGNEERQFAIDSTSGQVTLIGKLDYEATPAYSLVIQAVDSGTIPLNSTCTLNIDILDENDNT
PSFLKSTLFVDVLENMRIGELVSSVTATDSDSGDNVDLYYSITGTNNHGTFSISPNTGSIFLAKKLDF
ETQSLYKLNITAKDQGRPPRSSTMSVVIHVRDFNDNPPSFPPGDIFKSIVENIPIGTSVISVTAHDPD
ADINGQLSYTIIQQMPRGNHFTIDEVKGTIYTNAEIDREFANLFELTVKANDQAVPIETRRYALKNVT
ILVTDLNDNVPMFISQNALAADPSAVIGSVLTTIMAADPDEGANGEIEYEIINGDTDTFIVDRYSGDL
RVASALVPSQLIYNLIVSATDLGPERRKSTTELTIILQGLDGPVFTQPKYITILKEGEPIGTNVISIE
AASPRGSEAPVEYYIVSVRCEEKTVGRLFTIGRHTGIIQTAAILDREQGACLYLVDVYAIEKSTAFPR
TQRAEVETTLQDINDNPPVFPTDMLDLTVEENIGDGSKIMQLTAMDADEVQMLSSHTLSLVGSLVAAI
LATDDDSGVNGEITYIVNEDDEDGIFFLNPITGVFNLTRLLDYEVQQYYILTVRAEDGGGQFTTIRVY
FNILDVNDNPPIFSLNSYSTSLMENLPVGSTVLVFNVTDADMMKAEIKMFFETSENKDTTYQNLWDTF
KAVCRGKFIALNAHKRKQERSKIDTLTSQLKELEKQEQTHSKASRRQEITKIRAELKDIETQKTLQKI
NESRSWFFERINKIDRPLARLIKKKTEKNQIDAIKNDKGDITIDPTEIQTTIREYCKHLYANKLENLE
EMDKFLDTYTLPRLNQEEVESLNRPITDSETVATINSLPTKKSPGPDGFTAEFYQMITTPVFAQALYK
VEINENTLTGTDIIQVFAADGDEGTNGQVRYGIVNGNTNQEFRIDSVTGAITVAKPLDREKTPTYHLT
VQATDRGSTPRTDTSTVSIVLLDINDFVPVFELSPYSVNVPENLGTLPRTILQTASPCVRFASASKAY
FTTIPEDAPTGTDVLLVNASDADASKNAVISYRIIGGNSQFTINPSTGQIITSALLDRETKDNYTLVV
VCSDAGSPEPLSSSTSVLVTVTDVHDNPPRFQHHPYVTHIPSPTLPGSFVFAVTVTDADIGPNSELHY
SLSGRNSEKFHIDPLRGAIMAAGPLNGASEVTFSVHVKDGGSFPKTDSTTVTVRFVNKADFPKVRAKE
QTFMFPENQPVSSLVTTTTGSSLRGEPMSYYIASGNLGNTFQIDQLTGQVSISQPLDFEKIQKYVVWI
EARDGGFPPFSSYEKLDITVLDVNDNAPIFKEDPFISEILENLSPRKILTVSAMDKDSGPNGQLDYEI
VNGNMENSFSINHATGEIRSVRPLDREKVSHYVLTIKSSDKGSPSQSTSVKVMINILDENDNAPRFSQ
IFSAHVPENSPLGYTVTRVTTSDEDIGINAISRYSIMDASLPFTINPSTGDIVISRPLNPEDTDRYRI
RVSAHDSGWTVSTDVTIFVTDTNDNAPRFSRTSYYLDCPELTEIGSKVTQVFATDPDEGSNGQVFYFI
KSQSEYFRINATTGEIFNKQILKYQNVTGFSNVNINRHSFIVTSSDRGKPSLISETTVTINIVDSNDN
APQFLKSKYFTPVTKNVKVGTKLIRVTAIDDKDFGLNSEVEYFISNDNHLGKFKLDNDTGWISVASSL

Further analysis of the NOV57a protein yielded the following properties shown in Table 57C.

TABLE 57C
Protein Sequence Properties NOV57a

| | |
|---|---|
| SignalP analysis: | Cleavage site between residues 43 and 44 |
| PSORT II analysis: | |

| PSG: | a new signal peptide prediction method | |
|---|---|---|
| | N-region: | length 11; pos. chg 2; neg. chg 2 |
| | H-region: | length 14; peak value 5.01 |
| | PSG score: | 0.61 |

| | |
|---|---|
| GvH: | von Heijne's method for signal seq. recognition |
| | GvH score (threshold: −2.1): −0.05 |
| | possible cleavage site: between 42 and 43 |

>>> Seems to have a cleavable signal peptide (1 to 42)

| ALOM: | Klein et al's method for TM region allocation | | |
|---|---|---|---|
| | Init position for calculation: 43 | | |
| | Tentative number of TMS(s) for the threshold 0.5: 4 | | |
| | INTEGRAL | Likelihood = | Transmembrane 1753-1769 |
| | | −0.59 | |
| | INTEGRAL | Likelihood = | Transmembrane 2193-2209 |
| | | 0.26 | |
| | INTEGRAL | Likelihood = | Transmembrane 3837-3853 |
| | | −11.25 | |
| | INTEGRAL | Likelihood = | Transmembrane 4488-4504 |
| | | −9.08 | |

| | |
|---|---|
| PERIPHERAL | Likelihood = 1.38 (at 1566) |

| | |
|---|---|
| | ALOM score: −11.25 (number of TMSs: 4) |
| MTOP: | Prediction of membrane topology (Hartmann et al.) |
| | Center position for calculation: 21 |
| | Charge difference: −0.5 C(1.0)-N(1.5) |
| | N >= C: N-terminal side will be inside |

>>> membrane topology: type 3a

| MITDISC: | discrimination of mitochondrial targeting seq | | | |
|---|---|---|---|---|
| | R content: | 0 | Hyd Moment(75): | 8.27 |
| | Hyd Moment(95): | 10.66 | G content: | 0 |
| | D/E content: | 2 | S/T content: | 0 |

| | |
|---|---|
| | Score: −6.21 |
| Gavel: | prediction of cleavage sites for mitochondrial preseq |
| | cleavage site motif not found |

NUCDISC: discrimination of nuclear localization signals

| | |
|---|---|
| | pat4: PKRR (4) at 228 |
| | pat4: HKRK (3) at 1918 |
| | pat4: KKPK (4) at 4521 |
| | pat4: KKKK (5) at 4526 |
| | pat4: KPRR (4) at 4869 |
| | pat7: PKRRGYL (5) at 228 |
| | pat7: PERRKST (5) at 1588 |
| | pat7: PKEKKKK (5) at 4523 |
| | bipartite: KKTGQIYVSGILDRKKE at 3227 |
| | bipartite: KKAKNPKEEKKPKEKKK at 4512 |
| | content of basic residues: 8.4% |
| | NLS Score: 3.22 |
| KDEL: | ER retention motif in the C-terminus: none |

ER Membrane Retention Signals: none
SKL: peroxisomal targeting signal in the C-terminus: none
PTS2: 2nd peroxisomal targeting signal: found
KIDTLTSQL at 1926
VAC: possible vacuolar targeting motif: found
TLPI at 3349
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 55.5 |
| COIL: | Lupas's algorithm to detect coiled-coil regions |
| 1912 I | 0.91 |
| 1913 A | 0.91 |
| 1914 L | 0.91 |
| 1915 N | 0.91 |
| 1916 A | 0.97 |
| 1917 H | 0.98 |
| 1918 K | 1.00 |
| 1919 R | 1.00 |
| 1920 K | 1.00 |
| 1921 Q | 1.00 |
| 1922 E | 1.00 |
| 1923 R | 1.00 |
| 1924 S | 1.00 |
| 1925 K | 1.00 |
| 1926 I | 1.00 |
| 1927 D | 1.00 |
| 1928 T | 1.00 |
| 1929 L | 1.00 |
| 1930 T | 1.00 |
| 1931 S | 1.00 |
| 1932 Q | 1.00 |
| 1933 L | 1.00 |
| 1934 K | 1.00 |
| 1935 E | 1.00 |
| 1936 L | 1.00 |
| 1937 E | 1.00 |
| 1938 K | 1.00 |
| 1939 Q | 1.00 |
| 1940 E | 1.00 |
| 1941 Q | 1.00 |
| 1942 T | 1.00 |
| 1943 H | 1.00 |
| 1944 S | 1.00 |
| 1945 K | 1.00 |
| 1946 A | 0.98 |
| 1947 S | 0.97 |
| 1948 R | 0.96 |
| 1949 R | 0.96 |
| 1950 Q | 0.96 |
| 1951 E | 0.96 |
| 1952 I | 0.92 |
| 1953 T | 0.73 |
| 1954 K | 0.59 |
| | total: 43 residues |

Final Results (k = 9/23):

```
47.8%: endoplasmic reticulum
21.7%: nuclear
13.0%: mitochondrial
  4.3%: vacuolar
  4.3%: Golgi
  4.3%: plasma membrane
  4.3%: vesicles of secretory system
```

>> prediction for CG92813-01 is end (k = 23)

A search of the NOV57a protein against the Geneseq database, a proprietary database that contains sequences published in patents and patent publication, yielded several homologous proteins shown in Table 57D.

TABLE 57D
Geneseq Results for NOV57a

| | | NOV57a | Identities/ | | |
|---|---|---|---|---|---|
| | | Residues/ | Similarities for | | |
| Geneseq | Protein/Organism/Length | Match | the Matched | | Expect |
| Identifier | [Patent #, Date] | Residues | Region | | Value |
| ABU12072 | Human NOV17a CG92813-01 protein | 1 . . . 4961 | 4892/4961 | (98%) | 0.0 |
| | SEQ ID 64 - Homo sapiens, 4903 aa. | 1 . . . 4903 | 4896/4961 | (98%) | |
| | [WO200281625-A2, 17-OCT-2002] | | | | |
| ABB59831 | Drosophila melanogaster polypeptide | 49 . . . 1878 | 748/1932 | (38%) | 0.0 |
| | SEQ ID NO 6285 - Drosophila | 71 . . . 1951 | 1066/1932 | (54%) | |
| | melanogaster, 5147 aa. | | | | |
| | [WO200171042-A2, 27-SEP-2001] | | | | |
| AAO26789 | Human cadherin (CAD) protein, SEQ | 364 . . . 3239 | 839/3033 | (27%) | 0.0 |
| | ID No 12 - Homo sapiens, 3354 aa. | 39 . . . 2911 | 1324/3033 | (42%) | |
| | [WO200299042-A2, 12-DEC-2002] | | | | |
| AAO26793 | Human cadherin (CAD) protein, SEQ | 5 . . . 1877 | 621/1950 | (31%) | 0.0 |
| | ID No 16 - Homo sapiens, 3298 aa. | 12 . . . 1883 | 925/1950 | (46%) | |
| | [WO200299042-A2, 12-DEC-2002] | | | | |
| ABB05430 | Human dachsous protein SEQ ID | 5 . . . 1877 | 621/1950 | (31%) | 0.0 |
| | NO: 2 - Homo sapiens, 3298 aa. | 12 . . . 1883 | 925/1950 | (46%) | |
| | [JP2001327295-A, 27-NOV-2001] | | | | |

In a BLAST search of public sequence databases, the NOV57a protein was found to have homology to the proteins shown in the BLASTP data in Table 57E.

TABLE 57E
Public BLASTP Results for NOV57a

| | | NOV57a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| P33450 | Cadherin-related tumor suppressor | 49 . . . 1878 | 748/1932 | (38%) | 0.0 |
| | precursor (Fat protein) - Drosophila | 71 . . . 1951 | 1066/1932 | (54%) | |
| | melanogaster (Fruit fly), 5147 aa. | | | | |
| IJFFTM | cadherin-related tumor suppressor | 49 . . . 1878 | 744/1932 | (38%) | 0.0 |
| | precursor - fruit fly (Drosophila | 71 . . . 1951 | 1065/1932 | (54%) | |
| | melanogaster), 5147 aa. | | | | |
| Q8CD68 | Hypothetical ATP/GTP-binding site | 4515 . . . 4961 | 422/448 | (94%) | 0.0 |
| | motif A - Mus musculus (Mouse), | 3 . . . 448 | 430/448 | (95%) | |
| | 448 aa. | | | | |
| Q96JQ0 | Protocadherin 16 precursor | 5 . . . 1877 | 621/1950 | (31%) | 0.0 |
| | (Cadherin 19) (Cadherin fibroblast | 12 . . . 1883 | 925/1950 | (46%) | |
| | 1) - Homo sapiens (Human), 3298 | | | | |
| | aa. | | | | |
| Q8BM82 | Hypothetical cadherin domain | 1384 . . . 1900 | 393/518 | (75%) | 0.0 |
| | containing protein - Mus musculus | 1 . . . 490 | 420/518 | (80%) | |
| | (Mouse), 575 aa. | | | | |

PFam analysis predicts that the NOV57a protein contains the domains shown in the Table 57F.

TABLE 57F
Domain Analysis of NOV57a

| | | Identities/ | | |
|---|---|---|---|---|
| Pfam | NOV57a | Similarities for | | Expect |
| Domain | Match Region | the Matched Region | | Value |
| cadherin | 47 . . . 126 | 19/107 | (18%) | 0.018 |
| | | 59/107 | (55%) | |
| cadherin | 140 . . . 241 | 35/113 | (31%) | 6.6e−15 |
| | | 77/113 | (68%) | |
| cadherin | 255 . . . 344 | 42/107 | (39%) | 4.5e−28 |
| | | 75/107 | (70%) | |
| cadherin | 363 . . . 466 | 44/113 | (39%) | 3.2e−23 |
| | | 79/113 | (70%) | |
| cadherin | 480 . . . 573 | 37/109 | (34%) | 9e−25 |
| | | 75/109 | (69%) | |
| cadherin | 588 . . . 680 | 46/108 | (43%) | 1.2e−26 |
| | | 76/108 | (70%) | |
| cadherin | 694 . . . 784 | 43/107 | (40%) | 1.5e−26 |
| | | 68/107 | (64%) | |
| cadherin | 798 . . . 884 | 33/107 | (31%) | 5.7e−18 |
| | | 66/107 | (62%) | |
| cadherin | 898 . . . 987 | 32/107 | (30%) | 7.5e−18 |
| | | 65/107 | (61%) | |
| cadherin | 1001 . . . 1071 | 24/107 | (22%) | 1.2e−08 |
| | | 58/107 | (54%) | |
| cadherin | 1085 . . . 1181 | 34/111 | (31%) | 7.6e−14 |
| | | 71/111 | (64%) | |
| cadherin | 1195 . . . 1286 | 39/107 | (36%) | 5e−27 |
| | | 76/107 | (71%) | |
| cadherin | 1300 . . . 1391 | 42/107 | (39%) | 5e−27 |
| | | 71/107 | (66%) | |
| cadherin | 1405 . . . 1500 | 38/108 | (35%) | 6.1e−19 |
| | | 73/108 | (68%) | |
| cadherin | 1506 . . . 1602 | 30/114 | (26%) | 2.3e−12 |
| | | 71/114 | (62%) | |
| cadherin | 1614 . . . 1711 | 28/112 | (25%) | 0.0069 |
| | | 63/112 | (56%) | |
| cadherin | 1754 . . . 1840 | 31/107 | (29%) | 2.4e−13 |
| | | 64/107 | (60%) | |
| cadherin | 2107 . . . 2198 | 44/107 | (41%) | 4.7e−31 |
| | | 76/107 | (71%) | |
| cadherin | 2244 . . . 2334 | 45/107 | (42%) | 2.6e−28 |
| | | 73/107 | (68%) | |
| cadherin | 2348 . . . 2436 | 33/107 | (31%) | 2.8e−12 |
| | | 66/107 | (62%) | |
| cadherin | 2449 . . . 2537 | 37/107 | (35%) | 4e−11 |
| | | 67/107 | (63%) | |
| cadherin | 2551 . . . 2641 | 36/107 | (34%) | 1.2e−25 |
| | | 72/107 | (67%) | |
| cadherin | 2654 . . . 2740 | 38/107 | (36%) | 2.7e−17 |
| | | 64/107 | (60%) | |
| cadherin | 2754 . . . 2851 | 32/116 | (28%) | 4e−16 |
| | | 75/116 | (65%) | |
| cadherin | 2865 . . . 2957 | 41/107 | (38%) | 1.26-15 |
| | | 68/107 | (64%) | |
| cadherin | 2971 . . . 3062 | 36/107 | (34%) | 3.2e−26 |
| | | 75/107 | (70%) | |
| cadherin | 3076 . . . 3164 | 36/108 | (33%) | 2.9e−21 |
| | | 67/108 | (62%) | |
| cadherin | 3180 . . . 3273 | 33/107 | (31%) | 1.7e−16 |
| | | 71/107 | (66%) | |
| cadherin | 3286 . . . 3378 | 37/107 | (35%) | 4.2e−28 |
| | | 79/107 | (74%) | |
| cadherin | 3390 . . . 3482 | 38/109 | (35%) | 7.7e−22 |
| | | 76/109 | (70%) | |
| EGF | 3743 . . . 3774 | 18/47 | (38%) | 2.2e−08 |
| | | 25/47 | (53%) | |
| EGF | 3781 . . . 3812 | 15/47 | (32%) | 9.2e−05 |
| | | 25/47 | (53%) | |
| laminin_G | 3890 . . . 4033 | 49/158 | (31%) | 6.4e−16 |
| | | 101/158 | (64%) | |
| EGF | 4148 . . . 4179 | 16/47 | (34%) | 0.014 |
| | | 24/47 | (51%) | |
| laminin_G | 4208 . . . 4322 | 46/156 | (29%) | 9e−05 |
| | | 85/156 | (54%) | |
| EGF | 4410 . . . 4442 | 16/47 | (34%) | 3.1e−06 |
| | | 26/47 | (55%) | |

### Example 58

The NOV58 clone was analyzed, and the nucleotide and encoded polypeptide sequences are shown in Table 58A.

TABLE 58A
NOV58 Sequence Analysis

| | | |
|---|---|---|
| NOV58a, CG93387-05 | SEQ ID NO:1143 | 5432 bp |
| DNA Sequence | ORF Start: ATG at 187 | ORF Stop: TAA at 3991 |

GGTGCCGAGCACTCCGGACTCTACGTGAACAACAACGGGATCATCTCCTTCCTGAAGGAGGTTTCTCA
GTTCACCCCAGTGGCCTTCCCCATTGCCAAGGACCGCTGCGTGGTGGCAGCCTTCTGGGCAGATGTGG
ACAACCGGCGTGCAGGCGACGTGTACTACCGGGAGGCCACCGACCCAGCCATGCTGCGCCGAGCCACG
GAGGACGTCAGGCACTACTTCCCCGAGCTCCTGGACTTCAATGCCACCTGGGTTTTTGTTGCCACCTG
GTACCGAGTGACCTTCTTTGGAGGCAGTTCCTCATCCCCTGTCAACACATTCCAGACTGTGCTCATCA
CAGACGGCAAGCTCTCCTTCACCATCTTCAACTATGAGTCCATCGTGTGGACCACAGGCACACACGCC
AGCAGCGGGGGCAACGCCACTGGCCTCGGGGGCATCGCAGCCCAGGCTGGCTTCAACGCAGGCGATGG
GCAGCGTTACTTCAGTATCCCCGGCTCGCGCACAGCAGACATGGCCGAGGTGGAGACCACCACCAACG
TGGGTGTGCCCGGGCGCTGGGCGTTCAGAATCGATGATGCCCAGGTGCGCGTGGGGGGCTGCGGCCAT
ACAACGTCCGTGTGCCTGGCCCTGCGCCCCTGCCTCAACGGCGGCAAGTGCATCGACGACTGCGTCAC
GGGCAACCCCTCCTACACCTGCTCCTGCCTCTCGGGCTTCACGGGGCGGAGGTGCCACCTGGACGTGA
ACGAATGTGCCTCCCAGCCCTGTCAGAATGGTGGGACCTGTACTCACGGCATCAACAGTTTCCGCTGC
CAGTGCCCGGCTGGCTTTGGGGGACCCACCTGTGAGACAGCCCAATCCCCCTGTGACACCAAAGAGTG
TCAACATGGTGGCCAGTGCCAGGTGGAGAACGGCTCTGCGGTGTGTGTGTGCCAGGCCGGATACACCG
GAGCAGCCTGCGAGATGGATGTGGACGACTGCAGCCCTGACCCCTGCCTGAATGGAGGCTCTTGTGTT
GACCTAGTGGGGAATTACACCTGCTTGTGTGCCGAGCCCTTCAAGGGACTTCGCTGTGAGACAGGAGA
CCATCCAGTGCCAGACGCCTGCCTCTCGGCCCCTTGCCACAATGGGGGCACCTGTGTGGATGCGGACC
AGGGCTACGTGTGCGAGTGCCCCGAAGGCTTCATGGGCCTGGACTGCAGGGAGAGAGTCCCCGATGAC
TGTGAGTGCCGCAACGGAGGCAGATGCCTGGGCGCCAACACCACCCTCTGCCAGTGCCCCCTGGGATT
CTTTGGGCTTCTCTGTGAATTTGAAATCACAGCCATGCCCTGCAACATGAACACACAGTGCCCAGATG
GGGGCTACTGCATGGAGCACGGCGGGAGCTACCTCTGCGTCTGCCACACCGACCACAATGCCAGCCAC
TCCCTGCCATCACCCTGCGACTCGGACCCCTGCTTCAACGGAGGCTCCTGCGATGCCCATGACGACTC
CTACACCTGCGAGTGCCCGCGCGGGTTCCACGGCAAGCACTGCGAGAAAGCCCGGCCACACCTGTGCA
GCTCAGGGCCCTGCCGGAACGGGGGCACGTGCAAGGAGGCGGGCGGCGAGTACCACTGCAGCTGCCCC
TACCGCTTCACTGGGAGGCACTGTGAGATCGGGAAGCCAGACTCGTGTGCCTCTGGCCCCTGTCACAA
CGGCGGCACCTGCTTCCACTACATTGGCAAATACAAGTGTGACTGTCCCCCAGGCTTCTCCGGGCGGC
ACTGCGAGATAGCCCCCTCCCCCTGCTTCCGGAGCCCGTGTGTGAATGGGGGCACCTGCGAGGACCGG
GACACGGATTTCTTCTGCCACTGCCAAGCAGGGTACATGGGACGCCGGTGCCAGGCAGAGGTGGACTG
CGGCCCCCCGGAGGAGGTGAAGCACGCCACACTGCGCTTCAACGGCACGCGGCTGGGCGCGGTGGCCC
TGTATGCATGTGACCGTGGCTACAGCCTGAGCGCCCCCAGCCGCATCCGGGTCTGCCAGCCACACGGT
GTCTGGAGTGAGCCTCCCCAGTGCCTTGAAATCGATGAGTGCCGGTCTCAGCCGTGCCTGCATGGGGG
CTCTTGTCAGGACCGCGTTGCTGGGTACCTGTGCCTCTGCAGCACAGGCTATGAGGGCGCCCACTGTG
AGCTGGAGAGGGATGAGTGCCGAGCTCACCCGTGCAGAAATGGAGGGTCCTGCAGGAACCTCCCAGGG
GCCTATGTCTGCCGGTGCCCTGCAGGCTTCGTTGGAGTCCACTGTGAGACAGAGGTGGACGCCTGCGA
CTCCAGCCCCTGCCAGCATGGAGGCCGGTGTGAGAGCGGCGGCGGGGCCTACCTGTGCGTCTGCCCAG
AGAGCTTCTTCGGCTACCACTGCGAGACAGTGAGTGACCCCTGCTTCTCCAGCCCCTGTGGGGGCCGT
GGCTATTGCCTGGCCAGCAACGGCTCCCACAGCTGCACCTGCAAAGTGGGCTACACGGGCGAGGACTG
CGCCAAAGAGCTCTTCCCACCGACGGCCCTCAAGATGGAGAGAGTGGAGGAGAGTGGGGTCTCTATCT
CCTGGAACCCGCCCAATGGTCCAGCCGCCAGGCAGATGCTTGATGGCTACGCGGTCACCTACGTCTCC
TCCGACGGCTCCTACCGCCGCACAGACTTTGTGGACAGGACCCGCTCCTCGCACCAGCTCCAGGCCCT
GGCGGCCGGCAGGGCCTACAACATCTCCGTCTTCTCAGTGAAGCGAAACAGTAACAACAAGAATGACA
TCAGCAGGCCTGCCGTGCTGCTGGCCCGCACGCGACCCCGCCCTGTGGAAGGCTTCGAGGTCACCAAT
GTGACGGCTAGCACCATCTCAGTGCAGTGGGCCCTGCACAGGATCCGCCATGCCACCGTCAGTGGGGT
CCGTGTGTCCATCCGCCACCCTGAGGCCCTCAGGGACCAGGCCACCGATGTGGACAGGAGTGTGGACA
GGTTCACCTTTAGGGCCCTGCTGCCTGGGAAGAGGTACACCATCCAGCTGACCACCCTCAGTGGGCTC
AGGGGAGAGGAGCACCCCACAGAGAGCCTGGCCACCGCGCCGACGCACGTGTGGACCCGGCCCCTGCC
CAGGCAGCTTGCTGGAGGCTTATGTCATCAATGTGACCACCAGCCAGAGCACCAAGAGCCGCTATGTC
CCCAACGGGAAGCTGGCGTCCTACACGGTGCGCGACCTGCTGCCGGGACGGCGGTACCAGCCCTCTGT
GATAGCAGTGCAGAGCACGGAGCTCGGGCCGCAGCAGCACCAGGGAGGACACCACCCTCGGGTGCTCA
AGAACAGACCGCCCCCGGCGCGCCTGCCGGAGCTGCGCCTGCTCAATGACCACAGCGCCCCCGAGACC
CCCACCCAGCCCCCCAGGTTCTCGGAGTTTGTGGACGGCAGAGGAAGAGTGAGCGCCAGGTTCGGTGG
CTCACCCAGCAAAGCAGCCACCGTGAGATCACAACCCACAGCCTCGGCGCAGCTCGAGAACATGGAGG
AAGCCCCCAAGCGGGTCAGCCCGGCCCTCCAGCTCCCTGAACACGGCAGCAAGGACATCGGAAACGTC
CCTGGCAACTGTTCAGAAAACCCCTGTCAGAACGGAGGCACTTGTGTGCCGGGCGCAGACGCCCACAG
CTGTGACTGCGGGCCAGGGTTCAAAGGCAGACGCTGCGAGCTCGCCTGTATAAAGGTGTCCCGCCCCT
GCACAAGGCTGTTCTCCGAGACAAAGGCCTTTCCAGTCTGGGAGGGAGGCGTCTGTCACCACGTGTAT
AAAAGAGTCTACCGAGTTCACCAAGACATCTGCTTCAAAGAGAGCTGTGAAAGCACAAGCCTCAAGAA
GACCCCAAACAGGAAACAAAGTAAGAGTCAGACACTGGAGAAATCTTAAGGATTTAAGACGTTCTTGT
TACACTCCACCAACCTCACGAGTTTCTAACACCCAGGAAGATGAGGTCTAAAAACTGGATGAAAAAGG
ACACCCTGAGAAAAGGTCCTAGCTGGAGTCAGTCCCCTCTGTGACCTCTCTCCTCAGGCCTCTAGAGG
ACAGATGGCCAGGCCTGTGCACACACCAGCCCACCCTGAGAGACCCCTCTGGGACCAACCACCTGTGA
GTCCTGCGATGCGTTTAAGCAGCCTGTGCCCTCACCCAAGCTGCAGTTCCTGAAGGTGTAGTCTGTGT
CTCTGCGGATGAGATGACAGCTCGCCATTCCCCGGAATCAGTGAGGCTGTCAGTCAGCCACGCTTCTG
CAGTATGCAGAAACCTGTTCTTAGACTCCAAAGCCAGAGAAAGAATTCTCCCTTCGAGGCCCAACAAA
TTGAGAAGGAACTGTGATGGACCACTTCCAAAACAGAGACGGGGGCAGGGGCTGAAGGGCAGAGACCA
GGTGATGTCAGAAGGAAAGCCGGGTTGCAGACACAGCCGCCCCTGCTCTGGTCCTCCAGCGTGTTTAT
GACGCTCGTGCAGGTCGACGAGCCATCCTATGGACTAGTTAACACTAAGGTGGAGTTCAGACTTTTTT
AGACAACGGCGCGACTGGCAGCCTTTCTCTATCAAGGGTCAGACGGTAAACGTTTTCAGCTTTGCAGA
CCAGAGGTCCCTGTGGCTACAGTAGCGCAGACACAGCCACAGGCATGTCATTGAATGGCTGCGGCTAT
GTTCCAATAAAAACTTATTTACAATAACAGGTGGTGGCCAAATTGGCCCATGGGCCTTATTTGGTGAA
CCCTGTTCTATGAGATCACCTAGGCTTCAGCCTTAAACAGTGGAAGCCATCCCCTGAATGACAAGTCA
CAAGGGTATCAAAGAAAGACCCCTGAATTTTCATGGAAAAAGCTATTCAGACCCCTGCTTGGAAAGCT
AAGGCACACTGCCACGAAGCAGCAAGGACGCCTTACAAGTCTCAGTGCAACAGAGATGGACACCTGGG
CTGGGCTGGACAATGTTTAAGGTTCCTTTTAGTCCATGACTCAAGTGATACTGTTTTAGGCTATCAGC
TAGTAAACACGATCTTAGACATCCCCATCTTTGTAAGCAGAACAGTACGGCACTTCACCACATCTGCT
TCCCACCATGCTTCTAAGCAGCTGTCTTCCCCCTGCTAATGTTACAACCAAAGCAGCCACCCCACCTC
CTCTCGTGTTGAGCCTCACGACCGCTGACCCAGCTGGAAAGCCAGCGCCCTGCCGCGTCACCCTGACT
CTGCTCAGAGCCAGCATTCCAGCCACAAAGAGGGCCTCCTTCCTTTCCTCTTTCATAAAAATGTTTTT
TGAAGAGTTAGAGTATATTTTAGGCTTTTTATCTTTATTAAAATTTCATGTGCATGTGTA

| | | | |
|---|---|---|---|
| NOV58a, CG93387-05 | SEQ ID NO:1144 | 1268 aa | MW at 136576.5 kD |
| Protein Sequence | | | |

MLRRATEDVRHYFPELLDFNATWVFVATWYRVTFFGGSSSSPVNTFQTVLITDGKLSFTIFNYESIVW
TTGTHASSGGNATGLGGIAAQAGFNAGDGQRYFSIPGSRTADMAEVETTTNVGVPGRWAFRIDDAQVR
VGGCGHTTSVCLALRPCLNGGKCIDDCVTGNPSYTCSCLSGFTGRRCHLDVNECASQPCQNGGTCTHG
INSFRCQCPAGFGGPTCETAQSPCDTKECQHGGQCQVENGSAVCVCQAGYTGAACEMDVDDCSPDPCL
NGGSCVDLVGNYTCLCAEPFKGLRCETGDHPVPDACLSAPCHNGGTCVDADQGYVCECPEGFMGLDCR
ERVPDDCECRNGGRCLGANTTLCQCPLGFFGLLCEFEITAMPCNMNTQCPDGGYCMEHGGSYLCVCHT
DHNASHSLPSPCDSDPCFNGGSCDAHDDSYTCECPRGFHGKHCEKARPHLCSSGPCRNGGTCKEAGGE
YHCSCPYRFTGRHCEIGKPDSCASGPCHNGGTCFHYIGKYKCDCPPGFSGRHCEIAPSPCFRSPCVNG
GTCEDRDTDFFCHCQAGYMGRRCQAEVDCGPPEEVKHATLRFNGTRLGAVALYACDRGYSLSAPSRIR
VCQPHGVWSEPPQCLEIDECRSQPCLHGGSCQDRVAGYLCLCSTGYEGAHCELERDECRAHPCRNGGS
CRNLPGAYVCRCPAGFVGVHCETEVDACDSSPCQHGGRCESGGGAYLCVCPESFFGYHCETVSDPCFS
SPCGGRGYCLASNGSHSCTCKVGYTGEDCAKELFPPTALKNERVEESGVSISWNPPNGPAARQMLDGY
AVTYVSSDGSYRRTDFVDRTRSSHQLQALAAGRAYNISVFSVKRNSNNKNDISRPAVLLARTRPRPVE
GFEVTNVTASTISVQWALHRIRHATVSGVRVSIRHPEALRDQATDVDRSVDRFTFRALLPGKRYTIQL
TTLSGLRGEEHPTESLATAPTHVWTRPLPPANLTAARVTATSAHVVWDAPTPGSLLEAYVINVTTSQS
TKSRYVPNGKLASYTVRDLLPGRRYQPSVIAVQSTELGPQQHQGGHHPRVLKNRPPPARLPELRLLND
HSAPETPTQPPRFSEFVDGRGRVSARFGGSPSKAATVRSQPTASAQLENNEEAPKRVSPALQLPEHGS
KDIGNVPGNCSENPCQNGGTCVPGADAHSCDCGPGFKGRRCELACIKVSRPCTRLFSETKAFPVWEGG
VCHHVYKRVYRVHQDICFKESCESTSLKKTPNRKQSKSQTLEKS

| | | |
|---|---|---|
| NOV58b, CG93387-01 | SEQ ID NO:1145 | 4401 bp |
| DNA Sequence | ORF Start: ATG at 187 | ORF Stop: TAA at 4051 |

GGTGCCGAGCACTCCGGACTCTACGTGAACAACAACGGGATCATCTCCTTCCTGAAGGAGGTTTCTCA
GTTCACCCCAGTGGCCTTCCCCATTGCCAAGGACCGCTGCGTGGTGGCAGCCTTCTGGGCAGATGTGC
ACAACCGGCGTGCAGGCGACGTGTACTACCGGGAGGCCACCGACCCAGCCATGCTGCGCCGAGCCACG
GAGGACGTCAGGCACTACTTCCCCGAGCTCCTGGACTTCAATGCCACCTGGGTTTTTGTTGCCACCTG
GTACCGAGTGACCTTCTTTGGAGGCAGTTCCTCATCCCCTGTCAACACATTCCAGACTGTGCTCATCA
CAGACGGCAAGCTCTCCTTCACCATCTTCAACTATGAGTCCATCGTGTGGACCACAGGCACACACGCC
AGCAGCGGGGGCAACGCCACTGGCCTCGGGGGCATCGCAGCCCAGGCTGGCTTCAACGCAGGCGATGG
GCAGCGTTACTTCAGTATCCCCGGCTCGCGCACAGCAGACATGGCCGAGGTGGAGACCACCACCAACG
TGGGTGTGCCCGGGCGCTGGGCGTTCAGAATCGATGATGCCCAGGTGCGCGTGGGGGGCTGCGGCCAT
ACAACGTCCGTGTGCCTGGCCCTGCGCCCCTGCCTCAACGGCGGCAAGTGCATCGACGACTGCGTCAC
GGGCAACCCCTCCTACACCTGCTCCTGCCTCTCGGGCTTCACGGGGCGGAGGTGCCACCTGGACGTGA
ACGAATGTGCCTCCCAGCCCTGTCAGAATGGTGGGACCTGTACTCACGGCATCAACAGTTTCCGCTGC
CAGTGCCCGGCTGGCTTTGGGGGACCCACCTGTGAGACAGCCCAATCCCCCTGTGACACCAAAGAGTG
TCAACATGGTGGCCAGTGCCAGGTGGAGAACGGCTCTGCGGTGTGTGTGTGCCAGGCCGGATACACCG
GAGCAGCCTGCGAGATGGATGTGGACGACTGCAGCCCTGACCCCTGCCTGAATGGAGGCTCTTGTGTT
GACCTAGTGGGGAATTACACCTGCTTGTGTGCCGAGCCCTTCAAGGGACTTCGCTGTGAGACAGGAGA
CCATCCAGTGCCAGACGCCTGCCTCTCGGCCCCTTGCCACAATGGGGGCACCTGTGTGGATGCGGACC
AGGGCTACGTGTGCGAGTGCCCCGAAGGCTTCATGGGCCTGGACTGCAGGGAGAGAGTCCCCGATGAC
TGTGAGTGCCGCAACGGAGGCAGATGCCTGGGCGCCAACACCACCCTCTGCCAGTGCCCCCTGGGATT
CTTTGGGCTTCTCTGTGAATTTGAAATCACAGCCATGCCCTGCAACATGAACACACAGTGCCCAGATG
GGGGCTACTGCATGGAGCACGGCGGGAGCTACCTCTGCGTCTGCCACACCGACCACAATGCCAGCCAC
TCCCTGCCATCACCCTGCGACTCGGACCCCTGCTTCAACGGAGGCTCCTGCGATGCCCATGACGACTC
CTACACCTGCGAGTGCCCGCGCGGGTTCCACGGCAAGCACTGCGAGAAAGCCCGGCCACACCTGTGCA
GCTCAGGGCCCTGCCGGAACGGGGGCACGTGCAAGGAGGCGGGCGGCGAGTACCACTGCAGCTGCCCC
TACCGCTTCACTGGGAGGCACTGTGAGATCGGGAAGCCAGACTCGTGTGCCTCTGGCCCCTGTCACAA
CGGCGGCACCTGCTTCCACTACATTGGCAAATACAAGTGTGACTGTCCCCCAGGCTTCTCCGGGCGGC
ACTGCGAGATAGCCCCCTCCCCCTGCTTCCGGAGCCCGTGTGTGAATGGGGGCACCTGCGAGGACCGG
GACACGGATTTCTTCTGCCACTGCCAAGCAGGGTACATGGGACGCCGGTGCCAGGCAGAGGTGGACTG
CGGCCCCCCGGAGGAGGTGAAGCACGCCACACTGCGCTTCAACGGCACGCGGCTGGGCGCGGTGGCCC
TGTATGCATGTGACCGTGGCTACAGCCTGAGCGCCCCCAGCCGCATCCGGGTCTGCCAGCCACACGGT
GTCTGGAGTGAGCCTCCCCAGTGCCTTGAAATCGATGAGTGCCGGTCTCAGCCGTGCCTGCATGGGGG
CTCTTGTCAGGACCGCGTTGCTGGGTACCTGTGCCTCTGCAGCACAGGCTATGAGGGCGCCCACTGTG
AGCTGGAGAGGGATGAGTGCCGAGCTCACCCGTGCAGAAATGGAGGGTCCTGCAGGAACCTCCCAGGG
CTCCAGCCCCTGCCAGCATGGAGGCCGGTGTGAGAGCGGCGGCGGGGCCTACCTGTGCGTCTGCCCAG
GCCTATGTCTGCCGGTGCCCTGCAGGCTTCGTTGGAGTCCACTGTGAGACAGAGGTGGACGCCTGCGA
AGAGCTTCTTCGGCTACCACTGCGAGACAGTGAGTGACCCCTGCTTCTCCAGCCCCTGTGGGGGCCGT
GGCTATTGCCTGGCCAGCAACGGCTCCCACAGCTGCACCTGCAAAGTGGGCTACACGGGCGAGGACTG
CGCCAAAGAGCTCTTCCCACCGACGGCCCTCAAGATGGAGAGAGTGGAGGAGAGTGGGGTCTCTATCT
CCTGGAACCCGCCCAATGGTCCAGCCGCCAGGCAGATGCTTGATGGCTACGCGGTCACCTACGTCTCC
TCCGACGGCTCCTACCGCCGCACAGACTTTGTGGACAGGACCCGCTCCTCGCACCAGCTCCAGGCCCT
GGCGGCCGGCAGGGCCTACAACATCTCCGTCTTCTCAGTGAAGCGAAACAGTAACAACAAGAATGACA
TCAGCAGGCCTGCCGTGCTGCTGGCCCGCACGCGACCCCGCCCTGTGGAAGGCTTCGAGGTCACCAAT
GTGACGGCTAGCACCATCTCAGTGCAGTGGGCCCTGCACAGGATCCGCCATGCCACCGTCAGTGGGGT
CCGTGTGTCCATCCGCCACCCTGAGGCCCTCAGGGACCAGGCCACCGATGTGGACAGGAGTGTGGACA
GGTTCACCTTTAGGGCCCTGCTGCCTGGGAAGAGGTACACCATCCAGCTGACCACCCTCAGTGGGCTC
AGGGGAGAGGAGCACCCCACAGAGAGCCTGGCCACCGCGCCGACGCACGTGTGGACCCGGCCCCTGCC
TCCAGCAAACCTGACCGCCGCCCGAGTCACTGCCACCTCTGCCCACGTGGTCTGGGATGCCCCGACTC
CAGGCAGCTTGCTGGAGGCTTATGTCATCAATGTGACCACCAGCCAGAGCACCAAGAGCCGCTATGTC
CCCAACGGGAAGCTGGCGTCCTACACGGTGCGCGACCTGCTGCCGGGACGGCGGTACCAGCCCTCTGT
GATAGCAGTGCAGAGCACGGAGCTCGGGCCGCAGCACAGCGAGCCCGCCCACCTCTACATCATCACCT
CCCCCAGGGATGGCGCTGACAGACGCTGGCACCAGGGAGGACACCACCCTCGGGTGCTCAAGAACAGA
CCGCCCCCGGCGCGCCTGCCGGAGCTGCGCCTGCTCAATGACCACAGCGCCCCCGAGACCCCCACCCA
GCCCCCCAGGTTCTCGGAGTTTGTGGACGGCAGAGGAAGAGTGAGCGCCAGGTTCGGTGGCTCACCCA
GCAAAGCAGCCACCGTGAGATCACAACCCACAGCCTCGGCGCAGCTCGAGAACATGGAGGAAGCCCCC
AAGCGGGTCAGCCCGGCCCTCCAGCTCCCTGAACACGGCAGCAAGGACATCGGAAACGTCCCTGGCAA
CTGTTCAGAAAACCCCTGTCAGAACGGAGGCACTTGTGTGCCGGGCGCAGACGCCCACAGCTGTGACT
GCGGGCCAGGGTTCAAAGGCAGACGCTGCGAGCTCGCCTGTATAAAGGTGTCCCGCCCCTGCACAAGG
CTGTTCTCCGAGACAAAGGCCTTTCCAGTCTGGGAGGGAGGCGTCTGTCACCACGTGTATAAAAGAGT
CTACCGAGTTCACCAAGACATCTGCTTCAAAGAGAGCTGTGAAAGCACAAGCCTCAAGAAGACCCCAA
ACAGGAAACAAAGTAAGAGTCAGACACTGGAGAAATCTTAAGAAAGAAGGAACAGGCAATGTAGAGAA
GCTGTCAAATGGTGGACTCCCAAACCGTTCCACCACTGCCTCAAAAAACATCTTGACCAGCAGAAGGT
GGAGCTCAATGAAGGGTCAAGAGCTCAGCGAAGGGTAACTAGGTGGAACTGAGAGAAACCACGTTCAC
AAACTGCGTAATGCGGACTTCCTGCCGCCCTGGAGACCCCTCAACTCTCTGTCCATGTAAGGCCCTTA
AAGAGATTCATAGGAACTTTGAGCATCCTTNAGATGTGAATATTGTTGGGGGCAGGATTGGGGGATAA
ATAGAAGGGAAGGCCACTCCACGAGTATCCCATGAACCTGGCCAGATCT

| | | | |
|---|---|---|---|
| NOV58b, CG93387-01 | SEQ ID NO:1146 | 1288 aa | MW at 138908.1 kD |
| Protein Sequence | | | |

MLRRATEDVRHYFPELLDFNATWVFVATWYRVTFFGGSSSSPVNTFQTVLITDGKLSFTIFNYESIVW
TTGTHASSGGNATGLGGIAAQAGFNAGDGQRYFSIPGSRTADMAEVETTTNVGVPGRWAFRIDDAQVR
VGGCGHTTSVCLALRPCLNGGKCIDDCVTGNPSYTCSCLSGFTGRRCHLDVNECASQPCQNGGTCTHG
INSFRCQCPAGFGGPTCETAQSPCDTKECQHGGQCQVENGSAVCVCQAGYTGAACEMDVDDCSPDPCL
NGGSCVDLVGNYTCLCAEPFKGLRCETGDHPVPDACLSAPCHNGGTCVDADQGYVCECPEGFMGLDCR
ERVPDDCECRNGGRCLGANTTLCQCPLGFFGLLCEFEITAMPCNMNTQCPDGGYCMEHGGSYLCVCHT
DHNASHSLPSPCDSDPCFNGGSCDAHDDSYTCECPRGFHGKHCEKARPHLCSSGPCRNGGTCKEAGGE
YHCSCPYRFTGRHCEIGKPDSCASGPCHNGGTCFHYIGKYKCDCPPGFSGRHCEIAPSPCFRSPCVNG
GTCEDRDTDFFCHCQAGYMGRRCQAEVDCGPPEEVKHATLRFNGTRLGAVALYACDRGYSLSAPSRIR
VCQPHGVWSEPPQCLEIDECRSQPCLHGGSCQDRVAGYLCLCSTGYEGAHCELERDECRAHPCRNGGS
CRNLPGAYVCRCPAGFVGVHCETEVDACDSSPCQHGGRCESGGGAYLCVCPESFFGYHCETVSDPCFS
SPCGGRGYCLASNGSHSCTCKVGYTGEDCAKELFPPTALKMERVEESGVSISWNPPNGPAARQMLDGY
AVTYVSSDGSYRRTDFVDRTRSSHQLQALAAGRAYNISVFSVKRNSNNKNDISRPAVLLARTRPRPVE
GFEVTNVTASTISVQWALHRIRHATVSGVRVSIRHPEALRDQATDVDRSVDRFTFRALLPGKRYTIQL
TTLSGLRGEEHPTESLATAPTHVWTRPLPPANLTAARVTATSAHVVWDAPTPGSLLEAYVINVTTSQS
TKSRYVPNGKLASYTVRDLLPGRRYQPSVIAVQSTELGPQHSEPAHLYIITSPRDGADRRWHQGGHHP
RVLKNRPPPARLPELRLLNDHSAPETPTQPPRFSEFVDGRGRVSARFGGSPSKAATVRSQPTASAQLE
NMEEAPKRVSPALQLPEHGSKDIGNVPGNCSENPCQNGGTCVPGADAHSCDCGPGFKGRRCELACIKV
SRPCTRLFSETKAFPVWEGGVCHHVYKRVYRVHQDICFKESCESTSLKKTPNRKQSKSQTLEKS

| | | |
|---|---|---|
| NOV58c, CG93387-02 | SEQ ID NO:1147 | 4413 bp |
| DNA Sequence | ORF Start: ATG at 181 | ORF Stop: TAG at 4405 |

GAGCACTCCGGACTCTACGTGAACAACAACGGGATCATCTCCTTCCTGAAGGAGGTTTCTCAGTTCAC
CCCAGTGGCCTTCCCCATTGCCAAGGACCGCTGCGTGGTGGCAGCCTTCTGGGCAGATGTGGACAACC
GGCGTGCAGGCGACGTGTACTACCGGGAGGCCACCGACCCAGCCATGCTGCGCCGAGCCACGGAGGAC
GTCAGGCACTACTTCCCCGAGCTCCTGGACTTCAATGCCACCTGGGTTTTTGTTGCCACCTGGTACCG
AGTGACCTTCTTTGGAGGCAGTTCCTCATCCCCTGTCAACACATTCCAGACTGTGCTCATCACAGACG
GCAAGCTCTCCTTCACCATCTTCAACTATGAGTCCATCGTGTGGACCACAGGCACACACGCCAGCAGC
GGGGGCAACGCCACTGGCCTCGGGGGCATCGCAGCCCAGGCTGGCTTCAACGCAGGCGATGGGCAGCG
TTACTTCAGTATCCCCGGCTCGCGCACAGCAGACATGGCCGAGGTGGAGACCACCACCATCGTGGTTG
TGCCCGGGCGCTGGGCGTTCATAATCGATGATGCCCAGGTGCGCGTGGGGGGCTGCGGCCATACAACG
TCCGTGTGCCTGGCCCTGCGCCCCTGCCTCAACGGCGGCAAGTGCATCGACGACTGCGTCACGGGCAA
CCCCTCCTACACCTGCTCCTGCCTCTCGGGCTTCACGGGGCGGAGGTGCCACCTGGACGTGAACGAAT
GTGCCTCCCAGCCCTGTCAGAATGGTGGGACCTGTACTCACGGCATCAACAGTTTCCGCTGCCAGTGC
CCGGCTGGCTTTGGGGGACCCACCTGTGAGACAGCCCAATCCCCCTGTGACACCAAAGAGTGTCAACA
TGGTGGCCAGTGCCAGGTGGAGAATGGCTCTGCGGTGTGTGTGTGCCAGGCCGGATACACCGGAGCAG
CCTGCGAGATGGATGTGGACGACTGCAGCCCTGACCCCTGCCTGAATGGAGGCTCTTGTGTTGACCTA
GTGGGGAATTACACCTGCTTGTGTGCCGAGCCCTTCAAGGGACTTCGCTGTGAGACAGGAGACCATCN
NCAGTGCCAGACGCCTGCCTCTCGGCCCCTTGCCACAATGGGGGCACCTGTGTGGATGCGGACCAGGG
CTACGTGTGCGAGTGCCCCGAAGGCTTCATGGGCCTGGACTGCAGGGAGAGAGTCCCCGATGACTGTG
AGTGCCGCAACGGAGGCAGATGCCTGGGCGCCAACACCACCCTCTGCCCAGTGCCCCCTGGGATTCTT
TGGGCTTCTCTGTGAATTTGAAATCACAGCCATGCCCTGCAACATGAACACACAGTGCCCAGATGGGG
GCTACTGCATGGAGCACGGCGGGAGCTACCTCTGCGTCTGCCACACCGACCACAATGCCAGCCACTCC
CTGCCATCACCCTGCGACTCGGACCCCTGCTTCAACGGAGGCTCCTGCGATGCCCATGACGACTCCTA
CACCTGCGAGTGCCCGCGCGGGTTCCACGGCAAGCACTGCGAGAAAGCCCGGCCACACCTGTGCAGCT
CAGGGCCCTGCCGGAACGGGGGCACGTGCAAGGAGGCGGGCGGCGACTACCACTGCAGCTGCCCCTAC
CGCTTCACTGGGAGGCACTGTGAGATCGGGAAGCCAGACTCGTGTGCCTCTGGCCCCTGTCACAACGG
CGGCACCTGCTTCCACTACATTGGCAAATACAAGTGTGACTGTCCCCCAGGCTTCTCCGGGCGGCACT
GCGAGATAGCCCCCTCCCCCTGCTTCCGGAGCCCGTGTGTGAATGGGGGCACCTGCGAGGACCGGGAC
ACGGATTTCTTCTGCCACTGCCAAGCAGGGTACATGGGACGCCGGTGCCAGGCAGAGGTGGACTGCGG
CCCCCCGGAGGAGGTGAAGCACGCCACACTGCGCTTCAACGGCACGCGGCTGGGCGCGGTGGCCCTGT
ATGCATGTGACCGTGGCTACAGCCTGAGCGCCCCCAGCCGCATCCGGGTCTGCCAGCCACACGGTGTC
TGGAAAATCGATGAGTGCCGGTCTCAGCCGTGCCTGCATGGGGGCTCTTGTCAGGACCGCGTTGCTGG
GTACCTGTGCCTCTGCAGCACAGGCTATGAGGGCGCCCACTGTGAGCTGGAGAGGGATGAGTGCCGAG
CTCACCCGTGCAGAAATGGAGGGTCCTGCAGGAACCTCCCAGGGGCCTATGTCTGCCGGTGCCCTGCA
GGCTTCGTTGGAGTCCACTGTGAGACAGAGGTGGACGCCTGCGACTCCAGCCCCTGCCAGCATGGAGG
CCGGTGTGAGAGCGGCGGCGGGGCCTACCTGTGCGTCTGCCCAGAGAGCTTCTTCGGCTACCACTGCG
AGACAGTGAGTGACCCCTGCTTCTCCAGCCCCTGTGGGGGCCGTGGCTATTGCCTGGCCAGCAACGGC
TCCCACAGCTGCACCTGCAAAGTGGGCTACACGGGCGAGGACTGCGCCAAAGAGCTCTTCCCACCGAC
GGCCCTCAAGATGGAGAGAGTGGAGGAGAGTGGGGTCTCTATCTCCTGGAACCCGCCCAATGGTCCAG
CCGCCAGGCAGATGCTTGATGGCTACGCGGTCACCTACGTCTCCTCCGACGGCTCCTACCGCCGCACA
GACTTTGTGGACAGGACCCGCTCCTCGCACCAGCTCCAGGCCCTGGCGGCCGGCAGGGCCTACAACAT
CTCCGTCTTCTCAGTGAAGCGAAACAGTAACAACAAGAATGACATCAGCAGGCCTGCCGTGCTGCTGG
CCCGCACGCGACCCCGCCCTGTGGAAGGCTTCGAGGTCACCAATGTGACGGCTAGCACCATCTCAGTG
CAGTGGGCCCTGCACAGGATCCGCCATGCCACCGTCAGTGGGGTCCGTGTGTCCATCCGCCACCCTGA
GGCCCTCAGGGACCAGGCCACCGATGTGGACAGGAGTGTGGACAGGTTCACCTTTAGGGCCCTGCTGC
CTGGGAAGAGGTACACCATCCAGCTGACCACCCTCAGTGGGCTCAGGGGAGAGGAGCACCCCACAGAG
AGCCTGGCCACCGCGCCGACGCACGTGTGGACCCGGCCCCTGCCTCCAGCAAACCTGACCGCCGCCCG
AGTCACTGCCACCTCTGCCCACGTGGTCTGGGATGCCCCGACTCCAGGCAGCTTGCTGGAGGCTTATG
TCATCAATGTGACCACCAGCCAGAGCACCAAGAGCCGCTATGTCCCCAACGGGAAGCTGGCGTCCTAC
ACGGTGCGCGACCTGCTGCCGGGACGGCGGTACCAGCTCTCTGTGATAGCAGTGCAGAGCACGGAGCT
CGGGCCGCAGCACAGCGAGCCCGCCCACCTCTACATCATCACCTCCCCCAGGGATGGCGCTGACAGAC
GCTGGCACCAGGGAGGACACCACCCTCGGGTGCTCAAGAACAGACCGCCCCCGGCGCGCCTGCCGGAG
CTGCGCCTGCTCAATGACCACAGCGCCCCCGAGACCCCCACCCAGCCCCCCAGGTTCTCGGAGCTTGT
GGACGGCAGAGGAAGAGTGAGCGCCAGGTTCGGTGGCTCACCCAGCAAAGCAGCCACCGTGAGATCAC
GTCCTGTCCCCTACATGATGAGCCCACCCCCACCGCCAGCGCAGTCTCCAGCCAGTGACCCCCACCCC
GACTGTGCACAAGGCGCGGGGCTCGTGGGCCGCCGGCAGCATGCACCTCCATGGCAGGAGGGGCAGCT
CGGACATCCGTGCTCCCTGAGATATAGAAGCACTCAAAAGGGTGGCCCCAGGACCATCCCGGGTGCAA
AGCAGCTGCGCCGTGTGGTCACCGCCTGGCTTCTCCTAGAACCCACAGCCTCGGCGCAGCTCGAGAAC
ATGGAGGAAGCCCCCAAGCGGGTCAGCCTGGCCCTCCAGCTCCCTGAACACGGCAGCAAGGACATCGG
AAGTTATGCAGGACCTGAACTGTCTCCTAGTCCGGGGCTCTGCCTCGTGAGGATCGAGGCCAGCACGT
CCCTGCAGGGCACCAAGCATCTGCTGAGCACCTGCAGCACACAAGCAAAGGAGCAGGGTGGAGCCTTC
ACGCTGCCGTGCCTGTGTGGACCAGTCCAGGGTGACCACGGGGTAGGTGAGGGAAAGCCTGTCTTCAC
AGACCACTCTCCAGCTGACGTCCCTGGCAACTGTTCAGAAAACCCCTGTCAGAACGGAGGCACTTGTG
TGCCGGGCGCAGACGCCCACAGCTGTGACTGCGGGCCAGGGTTCAAAGGCAGACGCTGCGAGCTCGGT
ATAAAAGAGTCTACCGAGTTCACCAAGACATCTGCTTCAAAGAGAGCTGTGAAAGCACAAGCCTCAAG
AAGACCCCAAACAGGTGCCTCTGGGGAGCAGGCCCATGCCGTGTCCTGCATGTAGNNNNNN

| | | | |
|---|---|---|---|
| NOV58c, CG93387-02 | SEQ ID NO:1148 | 1408 aa | MW at 150587.4 kD |
| Protein Sequence | | | |

MLRRATEDVRHYFPELLDFNATWVFVATWYRVTFFGGSSSSPVNTFQTVLITDGKLSFTIFNYESIVW
TTGTHASSGGNATGLGGIAAQAGFNAGDGQRYFSIPGSRTADMAEVETTTIVVVPGRWAFIIDDAQVR
VGGCGHTTSVCLALRPCLNGGKCIDDCVTGNPSYTCSCLSGFTGRRCHLDVNECASQPCQNGGTCTHG
INSFRCQCPAGFGGPTCETAQSPCDTKECQHGGQCQVENGSAVCVCQAGYTGAACEMDVDDCSPDPCL
NGGSCVDLVFNYTCLCAEPFKGLRCETGDHXQCQTPASRPLATMGAPVWMRTRATCASAPKASWAWTA
GRESPMTVSAATEADAWAPTPPSAWCPLGFFGLLCEFEITAMPCNMNTWCPDGGYCMEHGGSYLCVCH
TDHNASHSLPSPCDSDPCFNGGSCDAHDDSYTCECPRGFHGKHCEKARPHLCSSGPCRNGGTCKEAGG
EYHCSCPYRFTGRHCEIGKPDSCASGPCHNGGTCFHYIGKYKCDCPPGFSGRHCEIAPSPCFRSPCVN
GGTCEDRDTDFFCHCQAGYMGRRCQAEVDCGPPEEVKHATLRFNGTRLGAVALYACDRGYSLSAPSRI
RVCQPHGVWKIDECRSQPCLHGGSCQDRVAGYLCLCSTGYEGAHCELERDECRAHPCRNGGSCRNLPG
AYVCRCPAGFVGVHCETEVDACDSSPCQHGGRCESGGGAYLCVCPESFFGYHCETVSDPCFSSPCGGR
GYCLASNGSHSCTCKVGYTGEDCAKELFPPTALKMERVEESGVSISWNPPNGPAARQMLDGYAVTYVS
SDGSYRRTDFVDRTRSSHQLQALAAGPAYNISVFSVKRNSNNKNDISRPAVLLARTRPRPVEGFEVTN
VTASTISVQWALHRIRHATVSGVRVSIRHPEALRDQATDVDRSVDRFTFRALLPGKRYTIQLTTLSGL
RGEEHPTESLATAPTHVWTRPLPPANLTAARVTATSAHVVWDAPTPGSLLEAYVINVTTSQSTKSRYV
PNGKLASYTVRDLLPGRRYQLSVIAVQSTELGPQHSEPAHLYIITSPRDGADRRWHQGGHHPRVLKNR
PPPARLPELRLLNDHSAPETPTQPPRFSELVDGRGRVSARFGGSPSKAATVRSRPVPYMMSPPPPPAQ
SPASDPHPDCAQGAGLVGRRQHAPPWQEGQLGHPCSLRYRSTQKGGPRTIPGAKQLRRVVTAWLLLEP
TASAQLENMEEAPKRVSLALQLPEHGSKDIGSYAGPELSPSPGLCLVRIEASTSLQGTKHLLSTCSTQ
AKEQGGAFTLPCLCGPVQGDHGVGEGKPVFTDHSPADVPGNCSENPCQNGGTCVPGADAHSCDCGPGF
KGRRCELGIKESTEFTKTSASKRAVKAQASRPPQTGASGEQAHAVSCM

| | | |
|---|---|---|
| NOV58d, CG93387-03 | SEQ ID NO:1149 | 2877 bp |
| DNA Sequence | ORF Start: ATG at 6 | ORF Stop: TAA at 2868 |

GCTTCATGGGCCTGGACTGCAGGGAGAGAGTCCCCGATGACTGTGAGTGCCGCAACGGAGGCAGATGC
CTGGGCGCCAACACCACCCTCTGCCAGTGCCCCCTGGGATTCTTTGGGCTTCTCTGTGAATTTGAAAT
CACAGCCATGCCCTGCAACATGAACACACAGTGCCCAGATGGGGGCTACTGCATGGAGCACGGCGGGA
GCTACCTCTGCGTCTGCCACACCGACCACAATGCCAGCCACTCCCTGCCATCACCCTGCGACTCGGAC
CCCTGCTTCAACGGAGGCTCCTGCGATGCCCATGACGACTCCTACACCTGCGAGTGCCCGCGCGGGTT
CCACGGCAAGCACTGCGAGAAAGCCCGGCCACACCTGTGCAGCTCAGGGCCCTGCCGGAACGGGGGCA
CGTGCAAGGAGGCGGGCGGCGAGTACCACTGCAGCTGCCCCTACCGCTTCACTGGGAGGCACTGTGAG
ATCGGGAAGCCAGACTCGTGTGCCTCTGGCCCCTGTCACAACGGCGGCACCTGCTTCCACTACATTGG
CAAATACAAGTGTGACTGTCCCCCAGGCTTCTCCGGGCGGCACTGCGAGATAGCCCCCTCCCCCTGCT
TCCGGAGCCCGTGTGTGAATGGGGGCACCTGCGAGGACCGGGACACGGATTTCTTCTGCCACTGCCAA
GCAGGGTACATGGGACGCCGGTGCCAGGCAGAGGTGGACTGCGGCCCCCCGGAGGAGGTGAAGCACGC
CACACTGCGCTTCAACGGCACGCGGCTGGGCGCGGTGGCCCTGTATGCATGTGACCGTGGCTACAGCC
TGAGCGCCCCCAGCCGCATCCGGGTCTGCCAGCCACACGGTGTCTGGAGTGAGCCTCCCCAGTGCCTT
GAAATCGATGAGTGCCGGTCTCAGCCGTGCCTGCATGGGGGCTCTTGTCAGGACCGCGTTGCTGGGTA
CCTGTGCCTCTGCAGCACAGGCTATGAGGGCGCCCACTGTGAGCTGGAGAGGGATGAGTGCCGAGCTC
ACCCGTGCAGAAATGGAGGGTCCTGCAGGAACCTCCCAGGGGCCTATGTCTGCCGGTGCCCTGCAGGC
TTCGTTGGAGTCCACTGTGAGACAGAGGTGGACGCCTGCGACTCCAGCCCCTGCCAGCATGGAGGCCG
GTGTGAGAGCGGCGGCGGGGCCTACCTGTGCGTCTGCCCAGAGAGCTTCTTCGGCTACCACTGCGAGA
CAGTGAGTGACCCCTGCTTCTCCAGCCCCTGTGGGGGCCGTGGCTATTGCCTGGCCAGCAACGGCTCC
CACAGCTGCACCTGCAAAGTGGGCTACACGGGCGAGGACTGCGCCAAAGAGCTCTTCCCACCGACGGC
CCTCAAGATGGAGAGAGTGGAGGAGAGTGGGGTCTCTATCTCCTGGAACCCGCCCAATGGTCCAGCCG
CCAGGCAGATGCTTGATGGCTACGCGGTCACCTACGTCTCCTCCGACGGCTCCTACCGCCGCACAGAC
TTTGTGGACAGGACCCGCTCCTCGCACCAGCTCCAGGCCCTGGCGGCCGGCAGGGCCTACAACATCTC
CGTCTTCTCAGTGAAGCGAAACAGTAACAACAAGAATGACATCAGCAGGCCTGCCGTGCTGCTGGCCC
GCACGCGACCCCGCCCTGTGGAAGGCTTCGAGGTCACCAATGTGACGGCTAGCACCATCTCAGTGCAG
TGGGCCCTGCACAGGATCCGCCATGCCACCGTCAGTGGGGTCCGTGTGTCCATCCGCCACCCTGAGGC
CCTCAGGGACCAGGCCACCGATGTGGACAGGAGTGTGGACAGGTTCACCTTTAGGGCCCTGCTGCCTG
GGAAGAGGTACACCATCCAGCTGACCACCCTCAGTGGGCTCAGGGGAGAGGAGCACCCCACAGAGAGC
CTGGCCACCGCGCCGACGCACGTGTGGACCCGGCCCCTGCCTCCAGCAAACCTGACCGCCGCCCGAGT
CACTGCCACCTCTGCCCACGTGGTCTGGGATGCCCCGACTCCAGGCAGCTTGCTGGAGGCTTATGTCA
TCAATGTGACCACCAGCCAGAGCACCAAGAGCCGCTATGTCCCCAACGGGAAGCTGGCGTCCTACACG
GTGCGCGACCTGCTGCCGGGACGGCGGTACCAGCCCTCTGTGATAGCAGTGCAGAGCACGGAGCTCGG
GCCGCAGCACAGCGAGCCCGCCCACCTCTACATCATCACCTCCCCCAGGGATGGCGCTGACAGACGCT
GGCACCAGGGAGGACACCACCCTCGGGTGCTCAAGAACAGACCGCCCCCGGCGCGCCTGCCGGAGCTG
CGCCTGCTCAATGACCACAGCGCCCCCGAGACCCCCACCCAGCCCCCCAGGTTCTCGGAGTTTGTGGA
CGGCAGAGGAAGAGTGAGCGCCAGGTTCGGTGGCTCACCCAGCAAAGCAGCCACCGTGAGATCACAAC
CCACAGCCTCGGCGCAGCTCGAGAACATGGAGGAAGCCCCCAAGCGGGTCAGCCCGGCCCTCCAGCTC
CCTGAACACGGCAGCAAGGACATCGGAAACGTCCCTGGCAACTGTTCAGAAAACCCCTGTCAGAACGG
AGGCACTTGTGTGCCGGGCGCAGACGCCCACAGCTGTGACTGCGGGCCAGGGTTCAAAGGCAGACGCT
GCGAGCTCGCCTGTATAAAGGTGTCCCGCCCCTGCACAAGGCTGTTCTCCGAGACAAAGGCCTTTCCA
GTCTGGGAGGGAGGCGTCTGTCACCACGTGTATAAAAGAGTCTACCGAGTTCACCAAGACATCTGCTT
CAAAGAGAGCTGTGAAAGCACAAGCCTCAAGAAGACCCCAAACAGGAAACAAAGTAAGAGTCAGACAC
TGGAGAAATCTTAAGAAAGAA

| | | | |
|---|---|---|---|
| NOV58d, CG93387-03 | SEQ ID NO:1150 | 954 aa | MW at 103791.4 kD |
| Protein Sequence | | | |

MGLDCRERVPDDCECRNGGRCLGANTTLCQCPLGFFGLLCEFEITAMPCNNNTQCPDGGYCMEHGGSY
LCVCHTDHNASHSLPSPCDSDPCFNGGSCDAHDDSYTCECPRGFHGKHCEKARPHLCSSGPCRNGGTC
KEAGGEYHCSCPYRFTGRHCEIGKPDSCASGPCHNGGTCFHYIGKYKCDCPPGFSGRHCEIAPSPCFR
SPCVNGGTCEDRDTDFFCHCQAGYMGRRCQAEVDCGPPEEVKHATLRFNGTRLGAVALYACDRGYSLS
APSRIRVCQPHGVWSEPPQCLEIDECRSQPCLHGGSCQDRVAGYLCLCSTGYEGAHCELERDECRAHP
CRNGGSCRNLPGAYVCRCPAGFVGVHCETEVDACDSSPCQHGGRCESGGGAYLCVCPESFFGYHCETV
SDPCFSSPCGGRGYCLASNGSHSCTCKVGYTGEDCAKELFPPTALKMERVEESGVSISWNPPNGPAAR
QMLDGYAVTYVSSDGSYRRTDFVDRTRSSHQLQALAAGRAYNISVFSVKRNSNNKNDISRPAVLLART
RPRPVEGFEVTNVTASTISVQWALHRIRHATVSGVRVSIRHPEALRDQATDVDRSVDRFTFRALLPGK
RYTIQLTTLSGLRGEEHPTESLATAPTHVWTRPLPPANLTAARVTATSAHVVWDAPTPGSLLEAYVIN
VTTSQSTKSRYVPNGKLASYTVRDLLPGRRYQPSVIAVQSTELGPQHSEPAHLYIITSPRDGADRRWH
QGGHHPRVLKNRPPPARLPELRLLNDHSAPETPTQPPRFSEFVDGRGRVSARFGGSPSKAATVRSQPT
ASAQLENMEEAPKRVSPALQLPEHGSKDIGNVPGNCSENPCQNGGTCVPGADAHSCDCGPGFKGRRCE
LACIKVSRPCTRLFSETKAFPVWEGGVCHHVYKRVYRVHQDICFKESCESTSLKKTPNRKQS
KSQTLEKS

| | | |
|---|---|---|
| NOV58e, CG93387-04 | SEQ ID NO:1151 | j7459 bp |
| DNA Sequence | lORF Start: ATG at 182 | lORF Stop: TAA at 4046 |

GAGCACTCCGGACTCTACGTGAACAACAACGGGATCATCTCCTTCCTGAAGGAGGTTTCTCAGTTTCA
CCCCAGTGGCCTTCCCCATTGCCAAGGACCGCTGCGTGGTGGCAGCCTTCTGGGCAGATGTGGACAAC
CGGCGTGCAGGCGACGTGTACTACCGGGAGGCCACCGACCCAGCCATGCTGCGCCGAGCCACGGAGGA
CGTCAGGCACTACTTCCCCGAGCTCCTGGACTTCAATGCCACCTGGGTTTTTGTTGCCACCTGGTACC
GAGTGACCTTCTTTGGAGGCAGTTCCTCATCCCCTGTCAACACATTCCAGACTGTGCTCATCACAGAC
GGCAAGCTCTCCTTCACCATCTTCAACTATGAGTCCATCGTGTGGACCACAGGCACACACGCCAGCAG
CGGGGGCAACGCCACTGGCCTCGGGGGCATCGCAGCCCAGGCTGGCTTCAACGCAGGCGATGGGCAGC
GTTACTTCAGTATCCCCGGCTCGCGCACAGCAGACATGGCCGAGGTGGAGACCACCACCAACGTGGGT
GTGCCCGGGCGCTGGGCGTTCAGAATCGATGATGCCCAGGTGCGCGTGGGGGGCTGCGGCCATACAAC
GTCCGTGTGCCTGGCCCTGCGCCCCTGCCTCAACGGCGGCAAGTGCATCGACGACTGCGTCACGGGCA
ACCCCTCCTACACCTGCTCCTGCCTCTCGGGCTTCACGGGGCGGAGGTGCCACCTGGACGTGAACGAA
TGTGCCTCCCAGCCCTGTCAGAATGGTGGGACCTGTACTCACGGCATCAACAGTTTCCGCTGCCAGTG
CCCGGCTGGCTTTGGGGGACCCACCTGTGAGACAGCCCAATCCCCCTGTGACACCAAAGAGTGTCAAC
ATGGTGGCCAGTGCCAGGTGGAGAATGGCTCTGCGGTGTGTGTGTGCCAGGCCGGATACACCGGAGCA
GCCTGCGAGATGGATGTGGACGACTGCAGCCCTGACCCCTGCCTGAATGGAGGCTCTTGTGTTGACCT
AGTGGGGAATTACACCTGCTTGTGTGCCGAGCCCTTCAAGGGACTTCGCTGTGAGACAGGAGACCATC
CAGTGCCAGACGCCTGCCTCTCGGCCCCTTGCCACAATGGGGGCACCTGTGTGGATGCGGACCAGGGC
TACGTGTGCGAGTGCCCCGAAGGCTTCATGGGCCTGGACTGCAGGGAGAGAGTCCCCGATGACTGTGA
GTGCCGCAACGGAGGCAGATGCCTGGGCGCCAACACCACCCTCTGCCAGTGCCCCCTGGGATTCTTTG
GGCTTCTCTGTGAATTTGAAATCACAGCCATGCCCTGCAACATGAACACACAGTGCCCAGATGGGGGC
TACTGCATGGAGCACGGCGGGAGCTACCTCTGCGTCTGCCACACCGACCACAATGCCAGCCACTCCCT
GCCATCACCCTGCGACTCGGACCCCTGCTTCAACGGAGGCTCCTGCGATGCCCATGACGACTCCTACA
CCTGCGAGTGCCCGCGCGGGTTCCACGGCAAGCACTGCGAGAAAGCCCGGCCACACCTGTGCAGCTCA
GGGCCCTGCCGGAACGGGGGCACGTGCAAGGAGGCGGGCGGCGAGTACCACTGCAGCTGCCCCTACCG
CTTCACTGGGAGGCACTGTGAGATCGGGAAGCCAGACTCGTGTGCCTCTGGCCCCTGTCACAACGGCG
GCACCTGCTTCCACTACATTGGCAAATACAAGTGTGACTGTCCCCCAGGCTTCTCCGGGCGGCACTGC
GAGATAGCCCCCTCCCCCTGCTTCCGGAGCCCGTGTGTGAATGGGGGCACCTGCGAGGACCGGGACAC
GGATTTCTTCTGCCACTGCCAAGCAGGGTACATGGGACGCCGGTGCCAGGCAGAGGTGGACTGCGGCC
CCCCGGAGGAGGTGAAGCACGCCACACTGCGCTTCAACGGCACGCGGCTGGGCGCGGTGGCCCTGTAT
GCATGTGACCGTGGCTACAGCCTGAGCGCCCCCAGCCGCATCCGGGTCTGCCAGCCACACGGTGTCTG
GAGTGAGCCTCCCCAGTGCCTTGAAATCGATGAGTGCCGGTCTCAGCCGTGCCTGCATGGGGGCTCTT
GTCAGGACCGCGTTGCTGGGTACCTGTGCCTCTGCAGCACAGGCTATGAGGGCGCCCACTGTGAGCTG
GAGAGGGATGAGTGCCGAGCTCACCCGTGCAGAAATGGAGGGTCCTGCAGGAACCTCCCAGGGGCCTA
TGTCTGCCGGTGCCCTGCAGGCTTCGTTGGAGTCCACTGTGAGACAGAGGTGGACGCCTGCGACTCCA
GCCCCTGCCAGCATGGAGGCCGGTGTGAGAGCGGCGGCGGGGCCTACCTGTGCGTCTGCCCAGAGAGC
TTCTTCGGCTACCACTGCGAGACAGTGAGTGACCCCTGCTTCTCCAGCCCCTGTGGGGGCCGTGGCTA
TTGCCTGGCCAGCAACGGCTCCCACAGCTGCACCTGCAAAGTGGGCTACACGGGCGAGGACTGCGCCA
AAGAGCTCTTCCCACCGACGGCCCTCAAGATGGAGAGAGTGGAGGAGAGTGGGGTCTCTATCTCCTGG
AACCCGCCCAATGGTCCAGCCGCCAGGCAGATGCTTGATGGCTACGCGGTCACCTACGTCTCCTCCGA
CGGCTCCTACCGCCGCACAGACTTTGTGGACAGGACCCGCTCCTCGCACCAGCTCCAGGCCCTGGCGG
CCGGCAGGGCCTACAACATCTCCGTCTTCTCAGTGAAGCGAAACAGTAACAACAAGAATGACATCAGC
AGGCCTGCCGTGCTGCTGGCCCGCACGCGACCCCGCCCTGTGGAAGGCTTCGAGGTCACCAATGTGAC
GGCTAGCACCATCTCAGTGCAGTGGGCCCTGCACAGGATCCGCCATGCCACCGTCAGTGGGGTCCGTG
TGTCCATCCGCCACCCTGAGGCCCTCAGGGACCAGGCCACCGATGTGGACAGGAGTGTGGACAGGTTC
ACCTTTAGGGCCCTGCTGCCTGGGAAGAGGTACACCATCCAGCTGACCACCCTCAGTGGGCTCAGGGG
AGAGGAGCACCCCACAGAGAGCCTGGCCACCGCGCCGACGCACGTGTGGACCCGGCCCCTGCCTCCAG
CAAACCTGACCGCCGCCCGAGTCACTGCCACCTCTGCCCACGTGGTCTGGGATGCCCCGACTCCAGGC
AGCTTGCTGGAGGCTTATGTCATCAATGTGACCACCAGCCAGAGCACCAAGAGCCGCTATGTCCCCAA
CGGGAAGCTGGCGTCCTACACGGTGCGCGACCTGCTGCCGGGACGGCGGTACCAGCCCTCTGTGATAG
CAGTGCAGAGCACGGAGCTCGGGCCGCAGCACAGCGAGCCCGCCCACCTCTACATCATCACCTCCCCC
AGGGATGGCGCTGACAGACGCTGGCACCAGGGAGGACACCACCCTCGGGTGCTCAAGAACAGACCGCC
CCCGGCGCGCCTGCCGGAGCTGCGCCTGCTCAATGACCACAGCGCCCCCGAGACCCCCACCCAGCCCC
CCAGGTTCTCGGAGTTTGTGGACGGCAGAGGAAGAGTGAGCGCCAGGTTCGGTGGCTCACCCAGCAAA
GCAGCCACCGTGAGATCACAACCCACAGCCTCGGCGCAGCTCGAGAACATGGAGGAAGCCCCCAAGCG
GGTCAGCCCGGCCCTCCAGCTCCCTGAACACGGCAGCAAGGACGTCGGAAACGTCCCTGGCAACTGTT
CAGAAAACCCCTGTCAGAACGGAGGCACTTGTGTGCCGGGCGCAGACGCCCACAGCTGTGGCTGCGGG
CCAGGGTTCAAAGGCAGACGCTGCGAGCTCGCCTGTATAAAGGTGTCCCGCCCCTGCACAAGGCTGTT
CTCCGAGACAAAGGCCTTTCCAGTCTGGGAGGGAGGCGTCTGTCACCACGTGTATAAAAGAGTCTACC
GAGTTCACCAAGACATCTGCTTCAAAGAGAGCTGTGAAAGCACAAGCCTCAAGAAGACCCCAAACAGG
AAACAAAGTAAGAGTCAGACACTGGAGAAATCTTAAGGATTTAAGACGTTCTTGTTACACTCCACCAA
CCTCACGAGTTTCTAACACCCAGGAACATGACCTCTAAAAACTGGATGAAAAAGGACACCCTGAGAAA
AGGTCCTAGCTGGAGTCAGTCCCCTCTGTGACCTCTCTCCTCAGGCCTCTAGAGGACAGATGGCCAGG
CCTGTGCACACACCAGCCCACCCTGAGAGACCCCTCTGGGACCAACCACCTGTGAGTCCTGCGATGCC
TTTAAGCAGCCTGTGCCCTCACCCAAGCTGCAGTTCCTGAAGGTGTAGTCTGTGTCTCTGCGGATGAC
ATGACAGCTCGCCATTCCCCGGAATCAGTGAGGCTGTCAGTCAGCCACGCTTCTGCAGTATGCAGAAA
CCTGTTCTTAGACTCCAAAGCCAGAGAAAGAATTCTCCCTTCGAGGCCCAACAAATTGAGAAGGAACT
GTGATGGACCACTTCCAAAACAGAGACGGGGGCAGGGGCTGAAGGGCAGAGACCAGGTGATGTCAGAA
GGAAAGCCGGGTTGCAGACACAGCCGCCCCTGCTCTGGTCCTCCAGCGTGTTTATGACGCTCGTGCAG
GTCGACGAGCCATCCTATGGACTAGTTAACACTAAGGTGGAGTTCAGACTTTTTTAGACAACGGCGCC
ACTGGCAGCCTTTCTCTATCAAGGGTCAGACGGTAAACGTTTTCAGCTTTGCAGACCAGAGGTCCCTC
TGGCTACAGTAGCGCAGACACAGCCACAGGCATGTCATTGAATGGCTGCGGCTATGTTCCAATAAAAA
CTTATTTACAATAACAGGTGGTGGCCAAATTGGCCCATGGGCCTTATTTGGTGAACCCTGTTCTATGA
GATCACCTAGGCTTCAGCCTTAAACAGTGGAAGCCATCCCCTGAATGACAAGTCACAAGGGTATCAAA
GAAAGACCCCTGAATTTTCATGGAAAAAGCTATTCAGACCCCTGCTTGGAAAGCTAAGGCACACTGCC
ACGAAGCAGCAAGGACGCCTTACAAGTCTCAGTGCAACAGAGATGGACACCTGGGCTGGGCTGGACAA
TGTTTAAGGTTCCTTTTAGTCCATGACTCAAGTGATACTGTTTTAGGCTATCAGGTAGTAAACACGAT
CTTAGACATCCCCATCTTTGTAAGCAGAACAGTACGGCACTTCACCACATCTGCTTCCCACCATGCTT
CTAAGCAGCTGTCTTCCCCCTGCTAATGTTACAACCAAAGCAGCCACCCCACCTCCTCTCGTGTTGAG
CCTCACGACCGCTGACCCAGCTGGAAAGCCAGCGCCCTGCCGCGTCACCCTGACTCTGCTCAGAGCCA
GCATTCCAGCCACAAAGAGGGCCTCCTTCCTTTCCTCTTTCATAAAAATGTTTTTTGAAGAGTTAGAG
TATATTTTAGGCTTTTTATCTTTATTAAAATTTCATGTGCATGTGTCTGTGTATTCTGCAATTTGTCA
TTTTCAGAAAGAAGGAACAGGCAATTCGAGAAGTTTCACCTGTACTCCCGAGCTGTTCCCCAGGCTCC
AGACCCACTTGAGAGCAGAAGGTGGAGCTCAATGAAGGGTCTCGAGCTCAGCGAAGGGTCACTGGGTG
AACTGAGAGAAACCACGTTCACAAACGCGTACTGCGGACTTCCTGCCGCCCTGGGACCTGTCACTGTT
TGTCCATGTAAGCTACAGCATTACTAGCAGATGCTAAGATCGAGTGATATCACTGGAAAAGTAGGTGA
ATCCTACTAGGAAACTTTCTACTCCCTACTAGGACCTCAAGCCCCTCAGCCACACAGCAAATGCTAAT
ATGCTCCAGTGTTAGCTTAGAAGCCTTGTGTCAACAAGAACTGGCTCCTGAGTCCCAAGCTTGGTGCC
ACACAGCAAATGCTAATATGCTCCAGTGTTAGCTTAGAAGCCTTGTGTCAACAAGAACTGGCTCCTGA
GTCCCAAGCTTGGTGCCACACAGCAAATGCTAATATGCTCCAGTGTTAGCTTAGAAGCCTTGTGTCAA
CAAGAACTGGCTCCTGAGTCCCAAGTGCTGTCACAGGACTTGCCCATTGGGATGTTTTCCACATTAAA
TATCAAGTAAAAAGACTTCCTGGTGCTCAGGAATTACAGTTCGTTCTTGAAACATTCCAAAGAGGCCA
CCACAGCTTTTCCCATGTGGCTTCTTTTAAAAACTCAAATGGCTTCCTTGAAAATACTCAAAGTCCAC
CCAAGGAAATTAGTAATAATAGAATCAGAAAACTGTCAGGAGCATAAAGATTTCTGTATCAAAATGAA
AGAAGCAATCCTGAGTTGCTGAATTACCCATCTGCTAATGAAACCGGGATGGACTGATCATCTAACCA
AGTGCAGACTGAGGATTCTACTTAGTCCTCCGACTGGGTACAACAACAGCCTAGGTTCTAGGGAGGGT
GGCAGTGACCGGGATGCCACAATGGAAGAGAAAATGAAAACACTGGCACAGTGAAATGTCTCATTTCC
AAACAGTTTTGCCTATGGCCAAGCAAGGCAATAAAGACAAACTCTCCCTTTTCCCCATTGCGTGTGGG
CTGCCAGGTACAAGTAAGGGAATCTTTGCTGTGCCCACTGTCCTCCAGTAGAGAACCCAACAGGCAAC
GGCCCCACTCAGGTATCAGCTCACCTCCTGCACCTCCCCTTAGCAGGAACTCCTTCCACTGGCAAAGG
ACTGCCACTGCCATCTGACTCAACTGTGGCGATGTGGACGGAGTCACCGAGCTGCTTTTCTTTTGCAA
AACAAAAGTCTTTTTCTTTGCAGTCACGCTGTAAGACGAGGCTGCTGGAGAAAACAAAAGCACCTAGA
TTTCAGTGCTGAATCCCCACAATTGCATGCAGCTCACACCTACCAGGGGTATTCCAGTGCATAGGGGA
AAGGAACCCGGCTGAAAAACCAGCTCCTTATTTTTCTTTTAAATAAAATAATACGATCCTAAGTCCAT
TTACCATCTGAAGTTGTCACGAGTGAACAGTCACATTACTGTTGTGGACCAGGCCTTAGATGAGTTTC
TCAGGCTCAGCACTGACATTCTGGGCCGGATCATCCTCTCTTGTGGGACCATCCTGTGCACTGCAGGA
TGTTTCACAGCACCCCTGGCCTCTACCCACTAGAATCCTACAATCTACCAGATCCTAGGATCTAGTTG
ATCCTAGAATGCTACCAAGGAGACTTGAATTTTGGTCCCATCATCAAAAATGCCTTCTGCATCAATCC
TATGCCAGTTTCCCCTAAAAGAGGGCTAACTGGAATGTTCTAGGATGTACCAATCCTCCAGGACCCTC
TTAGAGCTCATGCCATCAGAGACAGGCCTCTACCTCAGGGATCACCCCGGCTGACATCAAATTCCTCT
TCTCTTTTCCCAACATTTCAAATTGTTCTTCGGACTCATTGAGTTCC

| | | | |
|---|---|---|---|
| NOV58e, CG93387-04 | SEQ ID NO:1152 | 1288 aa | MW at 138836.0 kD |
| Protein Sequence | | | |

MLRRATEDVRHYFPELLDFNATWVFVATWYRVTFFGGSSSSPVNTFQTVLITDGKLSFTIFNYESIVW
TTGTHASSGGNATGLGGIAAQAGFNAGDGQRYFSIPGSRTADMAEVETTTNVGVPGRWAFRIDDAQVR
VGGCGHTTSVCLALRPCLNGGKCIDDCVTGNPSYTCSCLSGFTGRRCHLDVNECASQPCQNGGTCTHG
INSFRCQCPAGFGGPTCETAQSPCDTKECQHGGQCQVENGSAVCVCQAGYTGAACEMDVDDCSPDPCL
NGGSCVDLVGNYTCLCAEPFKGLRCETGDHPVPDACLSAPCHNGGTCVDADQGYVCECPEGFMGLDCR
ERVPDDCECRNGGRCLGANTTLCQCPLGFFGLLCEFEITAMPCNMNTQCPDGGYCMEHGGSYLCVCHT
DHNASHSLPSPCDSDPCFNGGSCDAHDDSYTCECPRGFHGKHCEKARPHLCSSGPCRNGGTCKEAGGE
YHCSCPYRFTGRHCEIGKPDSCASGPCHNGGTCFHYIGKYKCDCPPGFSGRHCEIAPSPCFRSPCVNG
GTCEDRDTDFFCHCQAGYMGRRCQAEVDCGPPEEVKHATLRFNGTRLGAVALYACDRGYSLSAPSRIR
VCQPHGVWSEPPQCLEIDECRSQPCLHGGSCQDRVAGYLCLCSTGYEGAHCELERDECRAHPCRNGGS
CRNLPGAYVCRCPAGFVGVHCETEVDACDSSPCQHGGRCESGGGAYLCVCPESFFGYHCETVSDPCFS
SPCGGRGYCLASNGSHSCTCKVGYTGEDCAKELFPPTALKMERVEESGVSISWNPPNGPAARQMLDGY
AVTYVSSDGSYRRTDFVDRTRSSHQLQALAAGRAYNISVFSVKRNSNNKNDISRPAVLLARTRPRPVE
GFEVTNVTASTISVQWALHRIREATVSGVRVSIRHPEALRDQATDVDRSVDRFTFRALLPGKRYTIQL
TTLSGLRGEEHPTESLATAPTHVWTRPLPPANLTAARVTATSAHVVWDAPTPGSLLEAYVINVTTSQS
TKSRYVPNGKLASYTVRDLLPGRRYQPSVIAVQSTELGPQHSEPAHLYIITSPRDGADRRWHQGGHHP
RVLKNRPPPARLPELRLLNDHSAPETPTQPPRFSEFVDGRGRVSARFGGSPSKAATVRSQPTASAQLE
NMEEAPKRVSPALQLPEHGSKDVGNVPGNCSENPCQNGGTCVPGADAHSCGCGPGFKGRRCELACIKV
SRPCTRLFSETKAFPVWEGGVCHHVYKRVYRVHQDICFKESCESTSLKKTPNRKQSKSQTLEKS

| | | |
|---|---|---|
| NOV58f, SNP13382519 of | SEQ ID NO:1153 | 5432 bp |
| CG93387-05, DNA Sequence | ORF Start: ATG at 187 | ORF Stop: TAA at 3991 |
| | SNP Pos: 4223 | SNP Change: C to T |

GGTGCCGAGCACTCCGGACTCTACGTGAACAACAACGGGATCATCTCCTTCCTGAAGGAGGTTTCTCA
GTTCACCCCAGTGGCCTTCCCCATTGCCAAGGACCGCTGCGTGGTGGCAGCCTTCTGGGCAGATGTGG
ACAACCGGCGTGCAGGCGACGTGTACTACCGGGAGGCCACCGACCCAGCCATGCTGCGCCGAGCCACG
GAGGACGTCAGGCACTACTTCCCCGAGCTCCTGGACTTCAATGCCACCTGGGTTTTTGTTGCCACCTG
GTACCGAGTGACCTTCTTTGGAGGCAGTTCCTCATCCCCTGTCAACACATTCCAGACTGTGCTCATCA
CAGACGGCAAGCTCTCCTTCACCATCTTCAACTATGAGTCCATCGTGTGGACCACAGGCACACACGCC
AGCAGCGGGGGCAACGCCACTGGCCTCGGGGGCATCGCAGCCCAGGCTGGCTTCAACGCAGGCGATGG
GCAGCGTTACTTCAGTATCCCCGGCTCGCGCACAGCAGACATGGCCGAGGTGGAGACCACCACCAACG
TGGGTGTGCCCGGGCGCTGGGCGTTCAGAATCGATGATGCCCAGGTGCGCGTGGGGGGCTGCGGCCAT
ACAACGTCCGTGTGCCTGGCCCTGCGCCCCTGCCTCAACGGCGGCAAGTGCATCGACGACTGCGTCAC
GGGCAACCCCTCCTACACCTGCTCCTGCCTCTCGGGCTTCACGGGGCGGAGGTGCCACCTGGACGTGA
ACGAATGTGCCTCCCAGCCCTGTCAGAATGGTGGGACCTGTACTCACGGCATCAACAGTTTCCGCTGC
CAGTGCCCGGCTGGCTTTGGGGGACCCACCTGTGAGACAGCCCAATCCCCCTGTGACACCAAAGAGTG
TCAACATGGTGGCCAGTGCCAGGTGGAGAACGGCTCTGCGGTGTGTGTGTGCCAGGCCGGATACACCG
GAGCAGCCTGCGAGATGGATGTGGACGACTGCAGCCCTGACCCCTGCCTGAATGGAGGCTCTTGTGTT
GACCTAGTGGGGAATTACACCTGCTTGTGTGCCGAGCCCTTCAAGGGACTTCGCTGTGAGACAGGAGA
CCATCCAGTGCCAGACGCCTGCCTCTCGGCCCCTTGCCACAATGGGGGCACCTGTGTGGATGCGGACC
AGGGCTACGTGTGCGAGTGCCCCGAAGGCTTCATGGGCCTGGACTGCAGGGAGAGAGTCCCCGATGAC
TGTGAGTGCCGCAACGGAGGCAGATGCCTGGGCGCCAACACCACCCTCTGCCAGTGCCCCCTGGGATT
CTTTGGGCTTCTCTGTGAATTTGAAATCACAGCCATGCCCTGCAACATGAACACACAGTGCCCAGATG
GGGGCTACTGCATGGAGCACGGCGGGAGCTACCTCTGCGTCTGCCACACCGACCACAATGCCAGCCAC
TCCCTGCCATCACCCTGCGACTCGGACCCCTGCTTCAACGGAGGCTCCTGCGATGCCCATGACGACTC
CTACACCTGCGAGTGCCCGCGCGGGTTCCACGGCAAGCACTGCGAGAAAGCCCGGCCACACCTGTGCA
GCTCAGGGCCCTGCCGGAACGGGGGCACGTGCAAGGAGGCGGGCGGCGAGTACCACTGCAGCTGCCCC
TACCGCTTCACTGGGAGGCACTGTGAGATCGGGAAGCCAGACTCGTGTGCCTCTGGCCCCTGTCACAA
CGGCGGCACCTGCTTCCACTACATTGGCAAATACAAGTGTGACTGTCCCCCAGGCTTCTCCGGGCGGC
ACTGCGAGATAGCCCCCTCCCCCTGCTTCCGGAGCCCGTGTGTGAATGGGGGCACCTGCGAGGACCGG
GACACGGATTTCTTCTGCCACTGCCAAGCAGGGTACATGGGACGCCGGTGCCAGGCAGAGGTGGACTG
CGGCCCCCCGGAGGAGGTGAAGCACGCCACACTGCGCTTCAACGGCACGCGGCTGGGCGCGGTGGCCC
TGTATGCATGTGACCGTGGCTACAGCCTGAGCGCCCCCAGCCGCATCCGGGTCTGCCAGCCACACGGT
GTCTGGAGTGAGCCTCCCCAGTGCCTTGAAATCGATGAGTGCCGGTCTCAGCCGTGCCTGCATGGGGG
CTCTTGTCAGGACCGCGTTGCTGGGTACCTGTGCCTCTGCAGCACAGGCTATGAGGGCGCCCACTGTG
AGCTGGAGAGGGATGAGTGCCGAGCTCACCCGTGCAGAAATGGAGGGTCCTGCAGGAACCTCCCAGGG
GCCTATGTCTGCCGGTGCCCTGCAGGCTTCGTTGGAGTCCACTGTGAGACAGAGGTGGACGCCTGCGA
CTCCAGCCCCTGCCAGCATGGAGGCCGGTGTGAGAGCGGCGGCGGGGCCTACCTGTGCGTCTGCCCAG
AGAGCTTCTTCGGCTACCACTGCGAGACAGTGAGTGACCCCTGCTTCTCCAGCCCCTGTGGGGGCCGT
GGCTATTGCCTGGCCAGCAACGGCTCCCACAGCTGCACCTGCAAAGTGGGCTACACGGGCGAGGACTG
CGCCAAAGAGCTCTTCCCACCGACGGCCCTCAAGATGGAGAGAGTGGAGGAGAGTGGGGTCTCTATCT
CCTGGAACCCGCCCAATGGTCCAGCCGCCAGGCAGATGCTTGATGGCTACGCGGTCACCTACGTCTCC
TCCGACGGCTCCTACCGCCGCACAGACTTTGTGGACAGGACCCGCTCCTCGCACCAGCTCCAGGCCCT
GGCGGCCGGCAGGGCCTACAACATCTCCGTCTTCTCAGTGAAGCGAAACAGTAACAACAAGAATGACA
TCAGCAGGCCTGCCGTGCTGCTGGCCCGCACGCGACCCCGCCCTGTGGAAGGCTTCGAGGTCACCAAT
GTGACGGCTAGCACCATCTCAGTGCAGTGGGCCCTGCACAGGATCCGCCATGCCACCGTCAGTGGGGT
CCGTGTGTCCATCCGCCACCCTGAGGCCCTCAGGGACCAGGCCACCGATGTGGACAGGAGTGTGGACA
GGTTCACCTTTAGGGCCCTGCTGCCTGGGAAGAGGTACACCATCCAGCTGACCACCCTCAGTGGGCTC
AGGGGAGAGGAGCACCCCACAGAGAGCCTGGCCACCGCGCCGACGCACGTGTGGACCCGGCCCCTGCC
TCCAGCAAACCTGACCGCCGCCCGAGTCACTGCCACCTCTGCCCACGTGGTCTGGGATGCCCCGACTC
CAGGCAGCTTGCTGGAGGCTTATGTCATCAATGTGACCACCAGCCAGAGCACCAAGAGCCGCTATGTC
CCCAACGGGAAGCTGGCGTCCTACACGGTGCGCGACCTGCTGCCGGGACGGCGGTACCAGCCCTCTGT
GATAGCAGTGCAGAGCACGGAGCTCGGGCCGCAGCAGCACCAGGGAGGACACCACCCTCGGGTGCTCA
AGAACAGACCGCCCCCGGCGCGCCTGCCGGAGCTGCGCCTGCTCAATGACCACAGCGCCCCCGAGACC
CCCACCCAGCCCCCCAGGTTCTCGGAGTTTGTGGACGGCAGAGGAAGAGTGAGCGCCAGGTTCGGTGG
CTCACCCAGCAAAGCAGCCACCGTGAGATCACAACCCACAGCCTCGGCGCAGCTCGAGAACATGGAGG
AAGCCCCCAAGCGGGTCAGCCCGGCCCTCCAGCTCCCTGAACACGGCAGCAAGGACATCGGAAACGTC
CCTGGCAACTGTTCAGAAAACCCCTGTCAGAACGGAGGCACTTGTGTGCCGGGCGCAGACGCCCACAG
CTGTGACTGCGGGCCAGGGTTCAAAGGCAGACGCTGCGAGCTCGCCTGTATAAAGGTGTCCCGCCCCT
GCACAAGGCTGTTCTCCGAGACAAAGGCCTTTCCAGTCTGGGAGGGAGGCGTCTGTCACCACGTGTAT
AAAAGAGTCTACCGAGTTCACCAAGACATCTGCTTCAAAGAGAGCTGTGAAAGCACAAGCCTCAAGAA
GACCCCAAACAGGAAACAAAGTAAGAGTCAGACACTGGAGAAATCTTAAGGATTTAAGACGTTCTTGT
TACACTCCACCAACCTCACGAGTTTCTAACACCCAGGAAGATGAGGTCTAAAAACTGGATGAAAAAGC
ACACCCTGAGAAAAGGTCCTAGCTGGAGTCAGTCCCCTCTGTGACCTCTCTCCTCAGGCCTCTAGAGC
ACAGATGGCCAGGCCTGTGCACACACCAGCCCACCCTGAGAGACCCCTCTGGGACCAACCACCTGTGA
GTCCTGTGATGCGTTTAAGCAGCCTGTGCCCTCACCCAAGCTGCAGTTCCTGAAGGTGTAGTCTGTGT
CTCTGCGGATGAGATGACAGCTCGCCATTCCCCGGAATCAGTGAGGCTGTCAGTCAGCCACGCTTCTG
CAGTATGCAGAAACCTGTTCTTAGACTCCAAAGCCAGAGAAAGAATTCTCCCTTCGAGGCCCAACAAA
TTGAGAAGGAACTGTGATGGACCACTTCCAAAACAGAGACGGGGGCAGGGGCTGAAGGGCAGAGACCA
GGTGATGTCAGAAGGAAAGCCGGGTTGCAGACACAGCCGCCCCTGCTCTGGTCCTCCAGCGTGTTTAT
GACGCTCGTGCAGGTCGACGAGCCATCCTATGGACTAGTTAACACTAAGGTGGAGTTCAGACTTTTTT
AGACAACGGCGCGACTGGCAGCCTTTCTCTATCAAGGGTCAGACGGTAAACGTTTTCAGCTTTGCAGA
CCAGAGGTCCCTGTGGCTACAGTAGCGCAGACACAGCCACAGGCATGTCATTGAATGGCTGCGGCTAT
GTTCCAATAAAAACTTATTTACAATAACAGGTGGTGGCCAAATTGGCCCATGGGCCTTATTTGGTGAA
CCCTGTTCTATGAGATCACCTAGGCTTCAGCCTTAAACAGTGGAAGCCATCCCCTGAATGACAAGTCA
CAAGGGTATCAAAGAAAGACCCCTGAATTTTCATGGAAAAAGCTATTCAGACCCCTGCTTGGAAAGCT
AAGGCACACTGCCACGAAGCAGCAAGGACGCCTTACAAGTCTCAGTGCAACAGAGATGGACACCTGGG
CTGGGCTGGACAATGTTTAAGGTTCCTTTTAGTCCATGACTCAAGTGATACTGTTTTAGGCTATCAGG
TAGTAAACACGATCTTAGACATCCCCATCTTTGTAAGCAGAACAGTACGGCACTTCACCACATCTGCT
TCCCACCATGCTTCTAAGCAGCTGTCTTCCCCCTGCTAATGTTACAACCAAAGCAGCCACCCCACCTC
CTCTCGTGTTGAGCCTCACGACCGCTGACCCAGCTGGAAAGCCAGCGCCCTGCCGCGTCACCCTGACT
CTGCTCAGAGCCAGCATTCCAGCCACAAAGAGGGCCTCCTTCCTTTCCTCTTTCATAAAAATGTTTTT
TGAAGAGTTAGAGTATATTTTAGGCTTTTTATCTTTATTAAAATTTCATGTGCATGTGTA

| | | | |
|---|---|---|---|
| NOV58f, SNP13382519 of | SEQ ID NO:1154 | 1268 aa | MW at 136576.5 kD |
| CG93387-05, Protein Sequence | | | SNP Change: no change |

MLRRATEDVRHYFPELLDFNATWVFVATWYRVTFFGGSSSSPVNTFQTVLITDGKLSFTIFNYESIVW
TTGTHASSGGNATGLGGIAAQAGFNAGDGQRYFSIPGSRTADMAEVETTTNVGVPGRWAFRIDDAQVR
VGGCGHTTSVCIALRPCLNGGKCIDDCVTGNPSYTCSCLSGFTGRRCHLDVNECASQPCQNGGTCTHG
INSFRCQCPAGFGGPTCETAQSPCDTKECQHGGQCQVENGSAVCVCQAGYTGAACEMDVDDCSPDPCL
NGGSCVDLVGNYTCLCAEPFKGLRCETGDHPVPDACLSAPCHNGGTCVDADQGYVCECPEGFMGLDCR
ERVPDDCECRNGGRCLGANTTLCQCPLGFFGLLCEFEITAMPCNMNTQCPDGGYCMEHGGSYLCVCHT
DHNASHSLPSPCDSDPCFNGGSCDAHDDSYTCECPRGFHGKHCEKARPHLCSSGPCRNGGTCKEAGGE
YHCSCPYRFTGRHCEIGKPDSCASGPCHNGGTCFHYIGKYKCDCPPGFSGRHCEIAPSPCFRSPCVNG
GTCEDRDTDFFCHCQAGYMGRRCQAEVDCGPPEEVKHATLRFNGTRLGAVALYACDRGYSLSAPSRIR
VCQPHGVWSEPPQCLEIDECRSQPCLHGGSCQDRVAGYLCLCSTGYEGAHCELERDECRAHPCRNGGS
CRNLPGAYVCRCPAGFVGVHCETEVDACDSSPCQHGGRCESGGGAYLCVCPESFFGYHCETVSDPCFS
SPCGGRGYCLASNGSHSCTCKVGYTGEDCAKELFPPTALKMERVEESGVSISWNPPNGPAARQMLDGY
AVTYVSSDGSYRRTDFVDRTRSSHQLQALAAGRAYNISVFSVKRNSNNKNDISRPAVLLARTRPRPVE
GFEVTNVTASTISVQWALHRIRHATVSGVRVSIRHPEALRDQATDVDRSVDRFTFHALLPGKRYTIQL
TTLSGLRGEEHPTESLATAPTHVWTRPLPPANLTAARVTATSAHVVWDAPTPGSLLEAYVINVTTSQS
TKSRYVPNGKLASYTVRDLLPGRRYQPSVIAVQSTELGPQQHQGGHHPRVLKNRPPPARLPELRLLND
HSAPETPTQPPRFSEFVDGRGRVSARFGGSPSKAATVRSQPTASAQLENMEEAPKRVSPALQLPEHGS
KDIGNVPGNCSENPCQNGGTCVPGADAHSCDCGPGFKGRRCELACIKVSRPCTRLFSETKAFPVWEGG
VCHHVYKRVYRVHQDICFKESCESTSLKKTPNRKQSKSQTLEKS

A ClustalW comparison of the above protein sequences yields the following sequence alignment shown in Table 58B.

TABLE 58B
Comparison of the NOV58 protein sequences.

| | | |
|---|---|---|
| NOV58a | MLRRATEDVRHYFPELLDFNATWVFVATWYRVTFFGGSSSSPVNTFQTVLITDGKLSFTI | (SEQ ID NO:1144) |
| NOV58b | MLRRATEDVRHYFPELLDFNATWVFVATWYRVTFFGGSSSSPVNTFQTVLITDGKLSFTI | (SEQ ID NO:1146) |
| NOV58c | MLRRATEDVRHYFPELLDFNATWVFVATWYRVTFFGGSSSSPVNTFQTVLITDGKLSFTI | (SEQ ID NO:1148) |
| NOV58d | ------------------------------------------------------------ | (SEQ ID NO:1150) |
| NOV58e | MLRRATEDVRHYFPELLDFNATWVFVATWYRVTFFGGSSSSPVNTFQTVLITDGKLSFTI | (SEQ ID NO:1152) |
| NOV58a | FNYESIVWTTGTHASSGGNATGLGGIAAQAGFNAGDGQRYFSIPGSRTADMAEVETTTNV | |
| NOV58b | FNYESIVWTTGTHASSGGNATGLGGIAAQAGFNAGDGQRYFSIPGSRTADMAEVETTTNV | |
| NOV58c | FNYESIVWTTGTHASSGGNATGLGGIAAQAGFNAGDGQRYFSIPGSRTADMAEVETTTIV | |
| NOV58d | ------------------------------------------------------------ | |
| NOV58e | FNYESIVWTTGTHASSGGNATGLGGIAAQAGFNAGDGQRYFSIPGSRTADMAEVETTTNV | |
| NOV58a | GVPGRWAFRIDDAQVRVGGCGHTTSVCLALRPCLNGGKCIDDCVTGNPSYTCSCLSGFTG | |
| NOV58b | GVPGRWAFRIDDAQVRVGGCGHTTSVCLALRPCLNGGKCIDDCVTGNPSYTCSCLSGFTG | |
| NOV58c | VVPGRWAFIIDDAQVRVGGCGHTTSVCLALRPCLNGGKCIDDCVTGNPSYTCSCLSGFTG | |
| NOV58d | ------------------------------------------------------------ | |
| NOV58e | GVPGRWAFRIDDAQVRVGGCGHTTSVCLALRPCLNGGKCIDDCVTGNPSYTCSCLSGFTG | |
| NOV58a | RRCHLDVNECASQPCQNGGTCTHGINSFRCQCPAGFGGPTCETAQSPCDTKECQHGGQCQ | |
| NOV58b | RRCHLDVNECASQPCQNGGTCTHGINSFRCQCPAGFGGPTCETAQSPCDTKECQHGGQCQ | |
| NOV58c | RRCHLDVNECASQPCQNGGTCTHGINSFRCQCPAGFGGPTCETAQSPCDTKECQHGGQCQ | |
| NOV58d | ------------------------------------------------------------ | |
| NOV58e | RRCHLDVNECASQPCQNGGTCTHGINSFRCQCPAGFGGPTCETAQSPCDTKECQHGGQCQ | |
| NOV58a | VENGSAVCVCQAGYTGAACEMDVDDCSPDPCLNGGSCVDLVGNYTCLCAEPFKGLRCETG | |
| NOV58b | VENGSAVCVCQAGYTGAACEMDVDDCSPDPCLNGGSCVDLVGNYTCLCAEPFKGLRCETG | |
| NOV58c | VENGSAVCVCQAGYTGAACEMDVDDCSPDPCLNGGSCVDLVGNYTCLCAEPFKGLRCETG | |
| NOV58d | ------------------------------------------------------------ | |
| NOV58e | VENGSAVCVCQAGYTGAACEMDVDDCSPDPCLNGGSCVDLVGNYTCLCAEPFKGLRCETG | |
| NOV58a | DNPVPDACLSAPCHNGGTCVDADQGYVCE-CPEGFMGLDCRERVPDDCECRNGGRCLGAN | |
| NOV58b | DHPVPDACLSAPCHNGGTCVDADQGYVCE-CPEGFMGLDCRERVPDDCECRNGGRCLGAN | |
| NOV58c | DHXQCQTPASRPLATMGAPVWMRTRATCASAPKASWAWTAGRESPMTVSAATEADAWAPT | |
| NOV58d | -----------------------------------MGLDCRERVPDDCECRNGGRCLGAN | |
| NOV58e | DHPVPDACLSAPCHNGGTCVDADQGYVCE-CPEGFMGLDCRERVPDDCECRNGGRCLGAN | |
| NOV58a | TTLCQCPLGFFGLLCEFEITAMPCNMNTQCPDGGYCMEHGGSYLCVCHTDHNASHSLPSP | |
| NOV58b | TTLCQCPLGFFGLLCEFEITAMPCNMNTQCPDGGYCMEHGGSYLCVCHTDHNASHSLPSP | |
| NOV58c | PPSAQCPLGFFGLLCEFEITAMPCNMNTQCPDGGYCMEHGGSYLCVCHTDHNASHSLPSP | |
| NOV58d | TTLCQCPLGFFGLLCEFEITAMPCNMNTQCPDGGYCMEHGGSYLCVCHTDHNASHSLPSP | |
| NOV58e | TTLCQCPLGFFGLLCEFEITAMPCNMNTQCPDGGYCMEHGGSYLCVCHTDHNASHSLPSP | |
| NOV58a | CDSDPCFNGGSCDAHDDSYTCECPRGFHGKHCEKARPHLCSSGPCRNGGTCKEAGGEYHC | |
| NOV58b | CDSDPCFNGGSCDAHDDSYTCECPRGFHGKHCEKARPHLCSSGPCRNGGTCKEAGGEYHC | |
| NOV58c | CDSDPCFNGGSCDAHDDSYTCECPRGFHGKHCEKARPHLCSSGPCRNGGTCKEAGGEYHC | |
| NOV58d | CDSDPCFNGGSCDAHDDSYTCECPRGFHGKHCEKARPHLCSSGPCRNGGTCKEAGGEYHC | |
| NOV58e | CDSDPCFNGGSCDAHDDSYTCECPRGFHGKHCEKARPHLCSSGPCRNGGTCKEAGGEYHC | |
| NOV58a | SCPYRFTGRHCEIGKPDSCASGPCHNGGTCFHYIGKYKCDCPPGFSGRHCEIAPSPCFRS | |
| NOV58b | SCPYRFTGRHCEIGKPDSCASGPCHNGGTCFHYIGKYKCDCPPGFSGRHCEIAPSPCFRS | |
| NOV58c | SCPYRFTGRHCEIGKPDSCASGPCHNGGTCFHYIGKYKCDCPPGFSGRHCEIAPSPCFRS | |
| NOV58d | SCPYRFTGRHCEIGKPDSCASGPCHNGGTCFHYIGKYKCDCPPGFSGRHCEIAPSPCFRS | |
| NOV58e | SCPYRFTGRHCEIGKPDSCASGPCHNGGTCFHYIGKYKCDCPPGFSGRHCEIAPSPCFRS | |
| NOV58a | PCVNGGTCEDRDTDFFCHCQAGYMGRRCQAEVDCGPPEEVKHATLRFNGTRLGAVALYAC | |
| NOV58b | PCVNGGTCEDRDTDFFCHCQAGYMGRRCQAEVDCGPPEEVKHATLRFNGTRLGAVALYAC | |
| NOV58c | PCVNGGTCEDRDTDFFCHCQAGYMGRRCQAEVDCGPPEEVKHATLRFNGTRLGAVALYAC | |
| NOV58d | PCVNGGTCEDRDTDFFCHCQAGYMGRRCQAEVDCGPPEEVKHATLRFNGTRLGAVALYAC | |
| NOV58e | PCVNGGTCEDRDTDFFCHCQAGYMGRRCQAEVDCGPPEEVKHATLRFNGTRLGAVALYAC | |
| NOV58a | DRGYSLSAPSRIRVCQPHGVWSEPPQCLEIDECRSQPCLHGGSCQDRVAGYLCLCSTGYE | |
| NOV58b | DRGYSLSAPSRIRVCQPHGVWSEPPQCLEIDECRSQPCLHGGSCQDRVAGYLCLCSTGYE | |
| NOV58c | DRGYSLSAPSRIRVCQPHGVWK-------IDECRSQPCLHGGSCQDRVAGYLCLCSTGYE | |
| NOV58d | DRGYSLSAPSRIRVCQPHGVWSEPPQCLEIDECRSQPCLHGGSCQDRVAGYLCLCSTGYE | |
| NOV58e | DRGYSLSAPSRIRVCQPHGVWSEPPQCLEIDECRSQPCLHGGSCQDRVAGYLCLCSTGYE | |
| NOV58a | GAHCELERDECRAHPCRNGGSCRNLPGAYVCRCPAGFVGVHCETEVDACDSSPCQHGGRC | |
| NOV58b | GAHCELERDECRAHPCRNGGSCRNLPGAYVCRCPAGFVGVHCETEVDACDSSPCQHGGRC | |
| NOV58c | GAHCELERDECRAHPCRNGGSCRNLPGAYVCRCPAGFVGVHCETEVDACDSSPCQHGGRC | |
| NOV58d | GAHCELERDECRAHPCRNGGSCRNLPGAYVCRCPAGFVGVHCETEVDACDSSPCQHGGRC | |
| NOV58e | GAHCELERDECRAHPCRNGGSCRNLPGAYVCRCPAGFVGVHCETEVDACDSSPCQHGGRC | |
| NOV58a | ESGGGAYLCVCPESFFGYHCETVSDPCFSSPCGGRGYCLASNGSHSCTCKVGYTGEDCAK | |
| NOV58b | ESGGGAYLCVCPESFFGYHCETVSDPCFSSPCGGRGYCLASNGSHSCTCKVGYTGEDCAK | |
| NOV58c | ESGGGAYLCVCPESFFGYHCETVSDPCFSSPCGGRGYCLASNGSHSCTCKVGYTGEDCAK | |
| NOV58d | ESGGGAYLCVCPESFFGYHCETVSDPCFSSPCGGRGYCLASNGSHSCTCKVGYTGEDCAK | |
| NOV58e | ESGGGAYLCVCPESFFGYHCETVSDPCFSSPCGGRGYCLASNGSHSCTCKVGYTGEDCAK | |
| NOV58a | ELFPPTALKMERVEESGVSISWNPPNGPAARQMLDGYAVTYVSSDGSYRRTDFVDRTRSS | |
| NOV58b | ELFPPTALKMERVEESGVSISWNPPNGPAARQMLDGYAVTYVSSDGSYRRTDFVDRTRSS | |
| NOV58c | ELFPPTALKMERVEESGVSISWNPPNGPAARQMLDGYAVTYVSSDGSYRRTDFVDRTRSS | |
| NOV58d | ELFPPTALKMERVEESGVSISWNPPNGPAARQMLDGYAVTYVSSDGSYRRTDFVDRTRSS | |
| NOV58e | ELFPPTALKMERVEESGVSISWNPPNGPAARQMLDGYAVTYVSSDGSYRRTDFVDRTRSS | |
| NOV58a | HQLQALAAGRAYNISVFSVKRNSNNKNDISRPAVLLARTRPRPVEGFEVTNVTASTISVQ | |
| NOV58b | HQLQALAAGRAYNISVFSVKRNSNNKNDISRPAVLLARTRPRPVEGFEVTNVTASTISVQ | |
| NOV58c | HQLQALAAGRAYNISVFSVKRNSNNKNDISRPAVLLARTRPRPVEGFEVTNVTASTISVQ | |
| NOV58d | HQLQALAAGRAYNISVFSVKRNSNNKNDISRPAVLLARTRPRPVEGFEVTNVTASTISVQ | |
| NOV58e | HQLQALAAGRAYNISVFSVKRNSNNKNDISRPAVLLARTRPRPVEGFEVTNVTASTISVQ | |
| NOV58a | WALHRIRHATVSGVRVSIRHPEALRDQATDVDRSVDRFTFRALLPGKRYTIQLTTLSGLR | |
| NOV58b | WALHRIRHATVSGVRVSIRHPEALRDQATDVDRSVDRFTFRALLPGKRYTIQLTTLSGLR | |
| NOV58c | WALHRIRHATVSGVRVSIRHPFALRDQATDVDRSVDRFTFPALLPGKRYTIQLTTLSGLR | |
| NOV58d | WALHRIRHATVSGVRVSIRHPEALRDQATDVDRSVDRFTFRALLPGKRYTIQLTTLSGLR | |
| NOV58e | WALHRIRHATVSGVRVSIRHPEALRDQATDVDRSVDRFTFRALLPGKRYTIQLTTLSGLR | |
| NOV58a | GEEHPTESLATAPTHVWTRPLPPANLTAARVTATSAHVVWDAPTPGSLLEAYVINVTTSQ | |
| NOV58b | GEEHPTESLATAPTHVWTRPLPPANLTAARVTATSAHVVWDAPTPGSLLEAYVINVTTSQ | |
| NOV58c | GEEHPTESLATAPTHVWTRPLPPANLTAARVTATSAHVVWDAPTPGSLLEAYVINVTTSQ | |
| NOV58d | GEEHPTESLATAPTHVWTRPLPPANLTAARVTATSAHVVWDAPTPGSLLEAYVINVTTSQ | |
| NOV58e | GEEHPTESLATAPTHVWTRPLPPANLTAARVTATSAHVVWDAPTPGSLLEAYVINVTTSQ | |
| NOV58a | STKSRYVPNGKLASYTVRDLLPGRRYQPSVIAVQSTELGPQQ------------------ | |
| NOV58b | STKSRYVPNGKLASYTVRDLLPGRRYQPSVIAVQSTELGPQHSEPAHLYIITSPRDGADR | |
| NOV58c | STKSRYVPNGKLASYTVRDLLPGRRYQLSVIAVQSTELGPQHSEPAHLYIITSPRDGADR | |
| NOV58d | STKSRYVPNGKLASYTVRDLLPGRRYQPSVIAVQSTELGPQHSEPAHLYIITSPRDGADR | |
| NOV58e | STKSRYVPNGKLASYTVRDLLPGRRYQPSVIAVQSTELGPQHSEPAHLYIITSPRDGADR | |
| NOV58a | --HQGGHHPRVLKNRPPPARLPELRLLNDHSAPETPTQPPRFSEFVDGRGRVSARFGGSP | |
| NOV58b | RWHQGGHHPRVLKNRPPPARLPELRLLNDHSAPETPTQPPRFSEFVDGRGRVSARFGGSP | |
| NOV58c | RWHQGGHHPRVLKNRPPPARLPELRLLNDHSAPETPTQPPRFSELVDGRGRVSARFGGSP | |
| NOV58d | RWHQGGHHPRVLKNRPPPARLPELRLLNDHSAPETPTQPPRFSEFVDGRGRVSARFGGSP | |
| NOV58e | RWHQGGHHPRVLKNRPPPARLPELRLLNDHSAPETPTQPPRFSEFVDGRGRVSARFGGSP | |
| NOV58a | SKAATVRS---------------------------------------------------- | |
| NOV58b | SKAATVRS---------------------------------------------------- | |
| NOV58c | SKAATVRSRPVPYMMSPPPPPAQSPASDPHPDCAQGAGLVGRRQHAPPWQEGQLGHPCSL | |
| NOV58d | SKAATVRS---------------------------------------------------- | |
| NOV58e | SKAATVRS---------------------------------------------------- | |
| NOV58a | -----------------------------QPTASAQLENMEEAPKRVSPALQLPEHGSKD | |
| NOV58b | -----------------------------QPTASAQLENMEEAPKRVSPALQLPEHGSKD | |
| NOV58c | RYRSTQKGGPRTIPGAKQLRRVVTAWLLLEPTASAQLENMEEAPKRVSLALQLPEHGSKD | |
| NOV58d | -----------------------------QPTASAQLENMEEAPKRVSPALQLPEHGSKD | |
| NOV5Be | -----------------------------QPTASAQLENMEEAPKRVSPALQLPEHGSKD | |
| NOV58a | IG---------------------------------------------------------- | |
| NOV58b | IG---------------------------------------------------------- | |
| NOV58c | IGSYAGPELSPSPGLCLVRIEASTSLQGTKHLLSTCSTQAKEQGGAFTLPCLCGPVQGDH | |
| NOV58d | IG---------------------------------------------------------- | |
| NOV58e | VG---------------------------------------------------------- | |
| NOV58a | ---------------NVPGNCSENPCQNGGTCVPGADAHSCDCGPGFKGRRCELACIKVS | |
| NOV58b | ---------------NVPGNCSENPCQNGGTCVPGADAHSCDCGPGFKGRRCELACIKVS | |
| NOV58c | GVGEGKPVFTDHSPADVPGNCSENPCQNGGTCVPGADAHSCDCGPGFKGRRCELG-IKES | |
| NOV58d | ---------------NVPGNCSENPCQNGGTCVPGADAHSCDCGPGFKGRRCELACIKVS | |
| NOV58e | ---------------NVPGNCSENPCQNGGTCVPGADAHSCGCGPGFKGRRCELACIKVS | |
| NOV58a | RPCTRLFSETKAFPVWEGGVCHHVYKRVYRVHQDICFKESCESTSLKKTPNPKQSKSQTL | |
| NOV58b | RPCTRLFSETKAFPVWEGGVCHHVYKRVYRVHQDICFKESCESTSLKKTPNRKQSKSQTL | |
| NOV5Bc | TEFTKTSASKRAVKAQAS-----------RRPQTGASGEQAHAVSCM------------- | |
| NOV58d | RPCTRLFSETKAFPVWEGGVCHHVYKRVYRVHQDICFKESCESTSLKKTPNPKQSKSQTL | |
| NOV58e | RPCTRLFSETKAFPVWEGGVCHHVYKRVYRVHQDICFKESCESTSLKKTPNRKQSKSQTL | |
| NOV58a | EKS | |
| NOV58b | EKS | |
| NOV58c | --- | |
| NOV58d | EKS | |
| NOV58e | EKS | |

Further analysis of the NOV58a protein yielded the following properties shown in Table 58C.

TABLE 58C
Protein Sequence Properties NOV58a

| | |
|---|---|
| SignalP analysis: | Cleavage site between residues 41 and 42 |
| PSORT II analysis: | |

| PSG: | a new signal peptide prediction method | |
|---|---|---|
| | N-region: | length 10; pos. chg 3; neg. chg 2 |
| | H-region: | length 4; peak value −6.27 |
| | PSG score: | −10.68 |

| | |
|---|---|
| GvH: | von Heijne's method for signal seq. recognition |
| | GvH score (threshold: −2.1): −3.98 |
| | possible cleavage site: between 40 and 41 |

>>> Seems to have no N-terminal signal peptide

| ALOM: | Klein et al's method for TM region allocation | |
|---|---|---|
| | Init position for calculation: 1 | |
| | Tentative number of TMS(s) for the threshold 0.5: 0 | |
| | number of TMS(s) . . . fixed | |
| | PERIPHERAL | Likelihood = 2.12 (at 365) |

| | ALOM score: 2.12 (number of TMSs: 0) | | | |
|---|---|---|---|---|
| MITDISC: | discrimination of mitochondrial targeting seq | | | |
| | R content: | 2 | Hyd Moment(75): | 18.26 |
| | Hyd Moment(95): | 17.56 | G content: | 0 |
| | D/E content: | 2 | S/T content: | 1 |

| | |
|---|---|
| | Score: −1.90 |
| Gavel: | prediction of cleavage sites for mitochondrial preseq |
| | R-2 motif at 14 RRA\|TE |

NUCDISC: discrimination of nuclear localization signals

| | |
|---|---|
| | pat4: none |
| | pat7: PGFKGRR (3) at 1190 |
| | bipartite: none |
| | content of basic residues: 9.5% |
| | NLS Score: −0.22 |
| KDEL: | ER retention motif in the C-terminus: none |

ER Membrane Retention Signals:

XXRR-like motif in the N-terminus: LRRA
KKXX-like motif in the C-terminus: TLEK

SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear |
| | discrimination |
| | Prediction: nuclear |
| | Reliability: 89 |
| COIL: | Lupas's algorithm to detect coiled-coil regions |
| | total: 0 residues |

Final Results (k = 9/23):

```
52.2%: nuclear
34.8%: mitochondrial
  8.7%: cytoplasmic
  4.3%: cytoskeletal
```

>> prediction for CG93387-05 is nuc (k = 23)

A search of the NOV58a protein against the Geneseq database, a proprietary database that contains sequences published in patents and patent publication, yielded several homologous proteins shown in Table 58D.

TABLE 58D
Geneseq Results for NOV58a

| | | NOV58a | Identities/ | | |
|---|---|---|---|---|---|
| | | Residues/ | Similarities for | | |
| Geneseq | Protein/Organism/Length | Match | the Matched | | Expect |
| Identifier | [Patent #,Date] | Residues | Region | | Value |
| AAB82249 | Human insulin-responsive sequence | 261 . . . 1268 | 1004/1028 | (97%) | 0.0 |
| | DNA binding protein-1 - Homo | 1 . . . 1028 | 1004/1028 | (97%) | |
| | sapiens, 1028 aa. [WO200132873-A1, | | | | |
| | 10-MAY-2001] | | | | |
| AAB82247 | Rat insulin-responsive sequence DNA | 271 . . . 1253 | 797/1003 | (79%) | 0.0 |
| | binding protein-1 - Rattus sp, 1008 aa. | 1 . . . 1002 | 876/1003 | (86%) | |
| | [WO200132873-A1, 10-MAY-2001] | | | | |
| AAB42900 | Human ORFX ORF2664 polypeptide | 1 . . . 627 | 592/629 | (94%) | 0.0 |
| | sequence SEQ ID NO: 5328 - Homo | 61 . . . 689 | 593/629 | (94%) | |
| | sapiens, 694 aa. [WO200058473-A2, | | | | |
| | 05-OCT-2000] | | | | |
| AAB82251 | Rat insulin-responsive sequence DNA | 780 . . . 1253 | 368/494 | (74%) | 0.0 |
| | binding protein-1 (truncated) - Rattus | 1 . . . 493 | 414/494 | (83%) | |
| | sp, 499 aa. [WO200132873-A1, 10- | | | | |
| | MAY-2001] | | | | |
| AAB82250 | Human insulin-responsive sequence | 813 . . . 1161 | 344/369 | (93%) | 0.0 |
| | DNA binding protein-1 (variant) - Homo | 1 . . . 369 | 345/369 | (93%) | |
| | sapiens, 387 aa. [WO200132873-A1, | | | | |
| | 10-MAY-2001] | | | | |

In a BLAST search of public sequence databases, the NOV58a protein was found to have homology to the proteins shown in the BLASTP data in Table 58E.

TABLE 58E
Public BLASTP Results for NOV58a

| | | NOV58a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| Q8TER0 | FLJ00133 protein - Homo sapiens | 7 . . . 1268 | 1258/1282 | (98%) | 0.0 |
| | (Human), 1282 aa (fragment). | 1 . . . 1282 | 1258/1282 | (98%) | |
| AAO41836 | Secreted protein SST3 - Mus | 1 . . . 939 | 800/939 | (85%) | 0.0 |
| | musculus (Mouse), 1065 aa. | 126 . . . 1064 | 862/939 | (91%) | |
| Q8N369 | Hypothetical protein - Homo sapiens | 666 . . . 1252 | 549/607 | (90%) | 0.0 |
| | (Human), 600 aa. | 26 . . . 599 | 551/607 | (90%) | |
| Q8TEP7 | FLJ00146 protein - Homo sapiens | 706 . . . 1268 | 498/583 | (85%) | 0.0 |
| | (Human), 522 aa (fragment). | 1 . . . 522 | 499/583 | (85%) | |
| P10079 | Fibropellin I precursor (Epidermal | 140 . . . 777 | 261/679 | (38%) | e−145 |
| | growth factor-related protein 1) | 249 . . . 895 | 339/679 | (49%) | |
| | (UEGF-1) - Strongylocentrotus | | | | |
| | purpuratus (Purple sea urchin), 1064 | | | | |
| | aa. | | | | |

PFam analysis predicts that the NOV58a protein contains the domains shown in the Table 58F.

TABLE 58F
Domain Analysis of NOV58a

| | | Identities/ | |
|---|---|---|---|
| | | Similarities for | |
| Pfam | NOV58a | the Matched | Expect |
| Domain | Match Region | Region | Value |
| EGF | 147 . . . 183 | 16/47 (34%) | 3.4e−05 |
| | | 28/47 (60%) | |
| EGF | 190 . . . 221 | 15/47 (32%) | 6.9e−08 |
| | | 28/47 (60%) | |
| EGF | 228 . . . 259 | 13/47 (28%) | 1.4e−05 |
| | | 21/47 (45%) | |
| EGF | 266 . . . 297 | 17/47 (36%) | 1.5e−09 |
| | | 26/47 (55%) | |
| EGF | 308 . . . 339 | 18/47 (38%) | 2.9e−09 |
| | | 25/47 (53%) | |
| EGF | 420 . . . 451 | 17/47 (36%) | 4.3e−07 |
| | | 25/47 (53%) | |
| EGF | 459 . . . 490 | 16/47 (34%) | 1.5e−05 |
| | | 26/47 (55%) | |
| EGF | 498 . . . 529 | 18/47 (38%) | 4.9e−09 |
| | | 29/47 (62%) | |
| EGF | 536 . . . 567 | 15/47 (32%) | 4.7e−06 |
| | | 22/47 (47%) | |
| sushi | 573 . . . 626 | 16/64 (25%) | 3.8e−05 |
| | | 36/64 (56%) | |
| EGF | 632 . . . 663 | 14/47 (30%) | 7.8e−07 |
| | | 21/47 (45%) | |
| EGF | 670 . . . 701 | 17/47 (36%) | 3.3e−07 |
| | | 23/47 (49%) | |
| EGF | 708 . . . 739 | 13/47 (28%) | 1.4e−05 |
| | | 25/47 (53%) | |
| EGF | 746 . . . 777 | 13/47 (28%) | 3.5e−05 |
| | | 26/47 (55%) | |
| fn3 | 781 . . . 862 | 24/88 (27%) | 3.9e−08 |
| | | 60/88 (68%) | |
| fn3 | 880 . . . 963 | 18/87 (21%) | 2e−09 |
| | | 62/87 (71%) | |
| fn3 | 979 . . . 1061 | 27/86 (31%) | 9.3e−08 |
| | | 58/86 (67%) | |
| EGF | 1166 . . . 1197 | 17/47 (36%) | 4.2e−08 |
| | | 28/47 (60%) | |

### Example 59

The NOV59 clone was analyzed, and the nucleotide and encoded polypeptide sequences are shown in Table 59A.

TABLE 59A
NOV59 Sequence Analysis

| | | |
|---|---|---|
| NOV59a, CG93871-01 | SEQ ID NO: 1155 | 2018 bp |
| DNA Sequence | ORF Start: ATG at 101 | ORF Stop: TAA at 1937 |

CTCCCCACGGCGCCAGGAGGAGGGGCGAGGGCCGGCAGCCCCCTCTCCCGCGCGCGGCGCAGGAGCCC
AGCCCAGCCCGGGGGACCCGCCGCCGCCGGTCATGTGGGCCGGACTGCTCCTTCGGGCCGCCTGTGTC
GCGCTCCTGCTGCCGGGGGCACCAGCCCGAGGCTACACCGGGAGGAAGCCGCCCGGGCACTTCGCGGC
CGAGAGGCGCCGACTGGGCCCCCACGTCTGCCTCTCTGGGTTTGGGAGTGGCTGCTGCCCTGGCTGGG
CGCCCTCTATGGGTGGTGGGCACTGCACCCTGCGTCTCTGCTCCTTCGGCTGTGGGAGTGGCATCTGC
ATCGCTCCCAATGTCTGCTCCTGCCAGGATGGAGAGCAAGGGGCCACCTGCCCAGAAACCCATGGACC
ATGTGGGGAGTACGGCTGTGACCTTACCTGCAACCATGGAGGCTGTCAGGAGGTGGCCCGAGTGTGCC
CCGTGGGCTTCTCGATGACGGAGACAGCTGTTGGCATCAGGTGTGACATTGACGAATGTGTAACCTCC
TCCTGCGAGGGCCACTGTGTGAACACAGAAGGTGGGTTTGTGTGCGAGTGTGGGCCGGGCATGCAGCT
GTCTGCCGACCGCCACAGCTGCCAAGACACTGACGAATGCCTAGGGACTCCCTGTCAGCAGAGATGTA
AAAACAGCATTGGCAGCTACAAGTGTTCCTGTCGAACTGGCTTCCACCTTCATGGCAACCGGCACTCC
TGTGTAGATGTAAACGAGTGTCGGAGGCCATTGGAGAGGCGAGTCTGTCACCATTCCTGCCACAACAC
CGTGGGCAGCTTCCTATGCACATGCCGACCTGGCTTCAGGCTCCGAGCTGACCGCGTGTCCTGTGAAG
CTTTCCCGAAAGCCGTGCTGGCCCCATCTGCCATCCTGCAACCCCGGCAACACCCGTCCAAGATGCTT
CTGTTGCTTCCTGAGGCCGGCCGGCCTGCCCTGTCCCCAGGACATAGCCCTCCTTCTGGGGCTCCAGG
GCCCCCAGCCGGAGTCAGGACCACCCGCCTGCCATCTCCCACCCCACGACTACCCACATCCTCCCCTT
CTGCCCCTGTGTGGCTGCTGTCCACCCTGCTGGCCACCCCAGTGCCTACTGCCTCCCTGCTGGGGAAC
CTCAGACCCCCCTCACTCCTTCAGGGGGAGGTGATGGGGACCCCTTCCTCACCCAGGGGCCCTGAGTC
CCCCCGACTGGCAGCAGGGCCCTCTCCCTGCTGGCACCTGGGAGCCATGCATGAATCAAGGAGTCGCT
GGACAGAGCCTGGGTGTTCCCAGTGCTGGTGCGAGGATGGGAAGGTGACCTGTGAAAAGGTGAGGTGT
GAAGCTGCTTGTTCCCACCCAATTCCCTCCAGAGATGGTGGGTGCTGCCCATCGTGCACAGGTTGTTT
TCACAGTGGTGTCGTCCGAGCTGAAGGGGATGTGTTTTCACCTCCCAATGAGAACTGCACCGTCTGTG
TCTGTCTGGCTGGAAACGTGTCGTGCATGTTTCGTGAGTGTCCTTTTGGCCCGTGTGAGACCCCCCAT
AAAGACAGATGCTATTTCCACGGCCGGTGGTACGCAGACGGGGCTGTGTTCAGTGGGGGTGGTGACGA
GTGTACCACCTGTGTTTGCCAGAATGGGGAGGTGGAGTGCTCCTTCATGCCCTGCCCTGAGCTGGCCT
GCCCCCGAGAAGAGTGGCGGCTGGGCCCTGGGCAGTGTTGCTTCACCTGCCAGGAGCCCACACCCTCG
ACAGGTTGCTCTCTTGACGACAACGGGGTTGAGTTTCCGATTGGACAGATCTGGTCGCCTGGTGACCC
CTGTAGATGGCTCGGTGAGCTGCAAGAGGACAGACTGTGTGGACTCCTGCCCTCACCCGATCCGGATC
CCTGGACAGTGCTGCCCAGACTGTTCAGCAGGTAATCCCCTGCCTCTGCCCCAAGCCCCCAGGGCAGG
GCATCTCAGGCATCGGGCTCCTTAAGCCCTATACAGCCTTCATCTC

| | | | |
|---|---|---|---|
| NOV59a, CG93871-01 | SEQ ID NO: 1156 | 612 aa | MW at 65156.4kD |
| Protein Sequence | | | |

MWAGLLLRAACVALLLPGAPARGYTGRKPPGHFAAERRRLGPHVCLSGFGSGCCPGWAPSMGGGHCTL
RLCSFGCGSGICIAPNVCSCQDGEQGATCPETHGPCGEYGCDLTCNHGGCQEVARVCPVGFSMTETAV
GIRCDIDECVTSSCEGHCVNTEGGFVCECGPGMQLSADRHSCQDTDECLGTPCQQRCKNSIGSYKCSC
RTGFHLHGNRHSCVDVNECRRPLERRVCHHSCHNTVGSFLCTCRPGFRLRADRVSCEAFPKAVLAPSA
ILQPRQHPSKMLLLLPEAGRPALSPGHSPPSGAPGPPAGVRTTRLPSPTPRLPTSSPSAPVWLLSTLL
ATPVPTASLLGNLRPPSLLQGEVMGTPSSPRGPESPRLAAGPSPCWHLGAMHESRSRWTEPGCSQCWC
EDGKVTCEKVRCEAACSHPIPSRDGGCCPSCTGCFHSGVVRAEGDVFSPPNENCTVCVCLAGNVSCMF
RECPFGPCETPHKDRCYFHGRWYADGAVFSGGGDECTTCVCQNGEVECSFMPCPELACPREEWRLGPG
QCCFTCQEPTPSTGCSLDDNGVEFPIGQIWSPGDPCRWLGELQEDRLCGLLPSPDPDPWTVLPRLFSR

| | | |
|---|---|---|
| NOV59b, CG93871-05 | SEQ ID NO: 1157 | 2819 bp |
| DNA Sequence | ORF Start: ATG at 5 | ORF Stop: TAA at 2807 |

GGTCATGTGGGCCGGACTGCTCCTTCGGGCCGCCTGTGTCGCGCTCCTGCTGCCGGGGGCACCAGCCC
GAGGCTACACCGGGAGGAAGCCGCCCGGGCACTTCGCGGCCGAGAGACGCCGACTGGGCCCCCACGTC
TGCCTCTCTGGGTTTGGGAGTGGCTGCTGCCCTGGCTGGGCGCCCTCTATGGGTGGTGGGCACTGCAC
CCTGCCCCTCTGCTCCCTCGGCTGTGGGAGTGGCATCTGCATCGCTCCCAATGTCTGCTCCTGCCAGG
ATGGAGAGCAAGGGGCCACCTGCCCAGAAACCCATGGACCATGTGGGGAGTACGGCTGTGACCTTACC
TGCAACCATGGAGGCTGTCAGGAGGTGGCCCGAGTGTGCCCCGTGGGCTTCTCGATGACGGAGACAGC
TGTTGGCATCAGGTGTACAGACATTGACGAATGTGTAACCTCCTCCTGCGAGGGCCACTGTGTGAACA
CAGAAGACACTGACGAATGCCTAGGGACTCCCTGTCAGCAGAGATGTAAAAACAGCATTGGCAGCTAC
AAGTGTTCCTGTCGAACTGGCTTCCACCTTCATGGCAACCGGCACTCCTGTGTAGATGTAAACGAGTG
TCGGAGGCCATTGGAGAGGCGAGTCTGTCACCATTCCTGCCACAACACCGTGGGCAGCTTCCTATGCA
CATGCCGACCTGGCTTCAGGCTCCGAGCTGACCGCGTGTCCTGTGAAGCTTTCCCGAAAGCCGTGCTG
GCCCCATCTGCCATCCTGCAACCCCGGCAACACCCGTCCAAGATGCTTCTGTTGCTTCCTGAGGCCGG
CCGGCCTGCCCTGTCCCCAGGACATAGCCCTCCTTCTGGGGCTCCAGGGCCCCCAGCCGGAGTCAGGA
CCACCCGCCTGCCATCTCCCACCCCACGACTACCCACATCCTCCCCTTCTGCCCCTGTGTGGCTGCTG
TCCACCCTGCTGGCCACCCCAGTGCCTACTGCCTCCCTGCTGGGGAACCTCAGACCCCCCTCACTCCT
TCAGGGGGAGGTGATGGGGACCCCTTCCTCACCCAGGGGCCCTGAGTCCCCCCGACTGGCAGCAGGGC
CCTCTCCCTGCTGGCACCTGGGAGCCATGCATGAATCAAGGAGTCGCTGGACAGAGCCTGGGTGTTCC
CAGTGCTGGTGCGAGGACGGGAAGGTGACCTGTGAAAAGGTGAGGTGTGAAGCTGCTTGTTCCCACCC
AATTCCCTCCAGAGATGGTGGGTGCTGCCCATCGTGCACAGGCTGTTTTCACAGTGGTGTCGTCCGAG
CTGAAGGGGATGTGTTTTCACCTCCCAATGAGAACTGCACCGTCTGTGTCTGTCTGGCTGGAAACGTG
TCCTGCATCTCTCCTGAGTGTCCTTCTGGCCCCTGTCAGACCCCCCCACAGACGGATTGCTGTACTTG
TGTTCCAGTGAGATGCTATTTCCACGGCCGGTGGTACGCAGACGGGGCTGTGTTCAGTGGGGGTGGTG
ACGAGTGTACCACCTGTGTTTGCCAGAATGGGGAGGTGGAGTGCTCCTTCATGCCCTGCCCTGAGCTG
GCCTGCCCCCGAGAAGAGTGGCGGCTGGGCCCTGGGCAGTGTTGCTTCACCTGCCAGGAGCCCACACC
CTCGACAGGCTGCTCTCTTGACGACAACGGGGTTGAGTTTCCGATTGGACAGATCTGGTCGCCTGGTG
ACCCCTGTGAGTTATGCATCTGCCAGGCAGATGGCTCGGTGAGCTGCAAGAGGACAGACTGTGTGGAC
TCCTGCCCTCACCCGATCCGGATCCCTGGACAGTGCTGCCCAGACTGTTCAGCAGGCTGCACCTACAC
AGGCAGAATCTTCTATAACAACGAGACCTTCCCGTCTGTGCTGGACCCATGTCTGAGCTGCATCTGCC
TGCTGGGCTCAGTGGCCTGTTCCCCCGTGGACTGCCCCATCACCTGTACCTACCCTTTCCACCCTGAC
GGGGAGTGCTGCCCCGTGTGCCGAGACTGCAACTACGAGGGAAGGAAGGTGGCGAATGGCCAGGTGTT
CACCTTGGATGATGAACCCTGCACCCGGTGCACGTGCCAGCTGGGAGAGGTGAGCTGTGAGAAGGTTC
CCTGCCAGCGGGCCTGTGCCGACCCTGCCCTGCTTCCTGGGGACTGCTGCTCTTCCTGTCCAGATTCC
CTGTCTCCTCTGGAAGAAAAGCAGGGGCTCTCCCCTCACGGAAATGTGGCATTCAGCAAAGCTGGTCG
GAGCCTGCATGGAGACACTGAGGCCCCTGTCAACTGTAGCTCCTGTCCTGGGCCCCCGACAGCATCAC
CCTCGAGGCCGGTGCTTCATCTCCTCCAGCTCCTTTTAAGAACGAACTTGATGAAAACACAGACTTTA
CCTACAAGCCCGGCAGGAGCTCATGGTCCACACTCACTCGCTTTGGGGCTGACAGCCACTTTCCCAGG
GGAGCCTGGGGCCTCCCCTCGACTCTCACCAGGGCCTTCGACCCCTCCAGGAGCCCCCACTCTACCTC
TAGCTTCCCCAGGGGCTCCTCAGCCACCTCCTGTGACTCCAGAGCGCTCGTTCTCAGCCTCTGGGGCC
CAGATAGTGTCCAGGTGGCCTCCTCTGCCTGGCACCCTCCTGACGGAAGCTTCAGCACTTTCCATGAT
GGACCCCAGCCCCTCGAAGACCCCCATCACCCTCCTCGGGCCTCGCGTGCTTTCTCCCACCACCTCTA
GACTCTCCACAGCCCTTGCAGCCACCACCCACCCTGGCCCCCAGCAGCCCCCAGTGGGGGCTTCTCGG
GGGGAAGAGTCCACCATGTAAGGAGGTCACT

| | | | |
|---|---|---|---|
| NOV59b, CG93871-05 | SEQ ID NO: 1158 | 934 aa | MW at 97715.0kD |
| Protein Sequence | | | |

MWAGLLLRAACVALLLPGAPARGYTGRKPPGHFAAERRRLGPRVCLSGFGSGCCPGWAPSMGGGHCTL
PLCSLGCGSGICIAPNVCSCQDGEQGATCPETHGPCGEYGCDLTCNHGGCQEVARVCPVGFSMTETAV
GIRCTDIDECVTSSCEGHCVNTEDTDECLGTPCQQRCKNSIGSYKCSCRTGFHLHGNRHSCVDVNECR
RPLERRVCHHSCHNTVGSFLCTCRPGFRLRADRVSCEAFPKAVLAPSAILQPRQHPSKMLLLLPEAGR
PALSPGHSPPSGAPGPPAGVRTTRLPSPTPRLPTSSPSAPVWLLSTLLATPVPTASLLGNLRPPSLLQ
GEVMGTPSSPRGPESPRIAAGPSPCWHLGAMHESRSRWTEPGCSQCWCEDGKVTCEKVRCEAACSHPI
PSRDGGCCPSCTGCFHSGVVRAEGDVFSPPNENCTVCVCLAGNVSCISPECPSGPCQTPPQTDCCTCV
PVRCYFHGRWYADGAVFSGGGDECTTCVCQNGEVECSFMPCPELACPRBEWRLGPGQCCFTCQEPTPS
TGCSLDDNGVEFPIGQIWSPGDPCELCICQADGSVSCKRTDCVDSCPHPIRIPGQCCPDCSAGCTYTG
RIFYNNETFPSVLDPCLSCICLLGSVACSPVDCPITCTYPFHPDGECCPVCRDCNYEGRKVANGQVFT
LDDEPCTRCTCQLGEVSCEKVPCQRACADPALLPGDCCSSCPDSLSPLEEKQGLSPHGNVAFSKAGRS
LHGDTEAPVNCSSCPGPPTASPSRPVLHLLQLLLRTNLMKTQTLPTSPAGAHGPHSLALGLTATFPGE
PGASPRLSPGPSTPPGAPTLPLASPGAPQPPPVTPERSFSASGAQIVSRWPPLPGTLLTEASALSMMD
PSPSKTPITLLGPRVLSPTTSRLSTALAATTHPGPQQPPVGASRGEESTM

| | | |
|---|---|---|
| NOV59c, 198488432 | SEQ ID NO: 1159 | 1814 bp |
| DNA Sequence | ORF Start: at 1 | ORF Stop: at 1813 |

GGTACCCGAGGCTACACCGGGAGGAAGCCGCCCGGGCACTTCGCGGCCGAGAGACGCCGACTGGGCCC
CCACGTCTGCCTCTCTGGGTTTGGGAGTGGCTGCTGCCCTGGCTGGGCGCCCTCTATGGGTGGTGGGC
ACTGCACCCTGCCCCCCTGCTCCTTCGGCTGTGGGAGTGGCATCTGCATCGCTCCCAATGTCTGCTCC
TGCCAGGATGGAGAGCAAGGGGCCACCTGCCCAGAAACCCATGGACCATGTGGGGAGTACGGCTGTGA
CCTTACCTGCAACCATGGAGGCTGTCAGGAGGTGGCCCGAGTGTGCCCCGTGGGCTTCTCGATGACGG
AGACAGCTGTTGGCATCAGGTGTACAGACATTGACGAATGTGTAACCTCCTCCTGCGAGGGCCACTGT
GTGAACACAGAAAGTGGGTTTGTGTGCGAGTGTGGGCCGGGCATGCAGCTGTCTGCCGACCGCCACAG
CTGCCAAGACACTGACGAATGCCTAGGGACTCCCTGTCAGCAGAGATGTAAAAACAGCATTGGCAGCT
ACAAGTGTTCCTGTCGAACTGGCTTCCACCTTCATGGCAACCGGCACTCCTGTGTAGATGTAAACGAG
TGTCGGAGGCCATTGGAGAGGCGAGTCTGTCACCATTCCTGCCACAACACCGTGGGCAGCTTCCTATG
CACATGCCGACCTGGCTTCAGGCTCCGAGCTGACCGCGTGTCCTGTGAAGCTTTCCCGAAAGCCGTGC
TGGCCCCATCTGCCATCCTGCAACCCCGGCAACACCCGTCCAAGATGCTTCTGTTGCTTCCTGAGGCC
GGCCGGCCTGCCCTGTCCCCAGGACATAGCCCTCCTTCTGGGGCTCCAGGGCCCCCAGCCGGAGTCAG
GACCACCCGCCTGCCATCTCCCACCCCACGACTACCCACATCCTCCCCTTCTGCCCCTGTGTGGCTGC
TGTCCACCCTGCTGGCCACCCCAGTGCCTACTGCCTCCCTGCTGGGGAACCTCAGACCCCCCTCACTC
CTTCAGGGGGAGGTGATGGGGACCCCTTCCTCACCCAGGGGCCCTGAGTCCCCCCGACTGGCAGCAGG
GCCCTCTCCCTGCTGGCACCTGGGAGCCATGTATGAATCAAGGAGTCGCTGGACAGAGCCTGGGTGTT
CCCAGTGTTGGTGCGAGGACGGGAAGGTGACCTGTGAAAAGGTGAGGTGTGAAGCTGCTTGTTCCCAC
CCAATTCCCTCCAGAGATGGTGGGTGCTGCCCATCGTGCACAGGCTGTTTTCACAGTGGTGTCGTCCG
AGCTGAAGGGGATGTGTTTTCACCTCCCAATGAGAACTGCACCGTCTGTGTCTGTCTGGCTGGAAACG
TGTCCTGCATCTCTCCTGAGTGTCCTTCTGGCCCCTGTCAGACCCCCCCACAGACGGATTGCTGTACT
TGTGTTCCAGTGAGATGCTATTTCCACGGCCGGTGGTACGCAGACGGGGCTGTGTTCAGTGGGGGTGG
TGACGAGTGTACCACCTGTGTTTGCCAGAATGGGGAGGTGGAGTGCTCCTTCATGCCCTGCCCTGAGC
TGGCCTGCCCCCGAGAAGAGTGGCGGCTGGGCCCTGGGCAGTGTTGCTTCACCTGCCAGGAGCCCACA
CCCTCGACAGGCTGCTCTCTTGACGACAACGGGGTTGAGTTTCCGATTGGACAGATCTGGTCGCCTGG
TGACCCCTGTGAGTTATGCATCTGCCAGGCAGATGGCTCGGTGAGCTGCAAGAGGACAGACTGTGTGG
ACTCCTGCCCTCACCCGATCCGGATCCCTGGACAGTGCTGCTCGAG

| | | | |
|---|---|---|---|
| NOV59c, 198488432 | SEQ ID NO: 1160 | 1604 aa | MW at 63737.5kD |
| Protein Sequence | | | |

GTRGYTGRKPPGHFAAERRRLGPHVCLSGFGSGCCPGWAPSMGGGHCTLPPCSFGCGSGICIAPNVCS
CQDGEQGATCPETHGPCGEYGCDLTCNHGGCQEVARVCPVGFSMTETAVGIRCTDIDECVTSSCEGHC
VNTESGFVCECGPGMQLSADRHSCQDTDECLGTPCQQRCKNSIGSYKCSCRTGFHLHGNRHSCVDVNE
CRRPLERRVCHHSCHNTVGSFLCTCRPGFRLRADRVSCEAFPKAVLAPSAILQPRQHPSKMLLLLPEA
GRPALSPGHSPPSGAPGPPAGVRTTRLPSPTPRLPTSSPSAPVWLLSTLLATPVPTASLLGNLRPPSL
LQGEVMGTPSSPRGPESPRLAAGPSPCWHLGAMYESRSRWTEPGCSQCWCEDGKVTCEKVRCEAACSH
PIPSRDGGCCPSCTGCFHSGVVRAEGDVFSPPNENCTVCVCLAGNVSCISPECPSGPCQTPPQTDCCT
CVPVRCYFHGRWYADGAVFSGGGDECTTCVCQNGEVECSFMPCPELACPREEWRLGPGQCCFTCQEPT
PSTGCSLDDNGVEFPIGQIWSPGDPCELCICQADGSVSCKRTDCVDSCPHPIRIPGQCCS

| | | |
|---|---|---|
| NOV59d, 198488424 | SEQ ID NO: 1161 | 1790 bp |
| DNA Sequence | ORF Start: at 1 | ORF Stop: at 1789 |

GGTACCCGAGGCTACACCGGGAGGAAGCCGCCCGGGCACTTCGCGGCCGAGAGACGCCGACTGGGCCC
CCACGTCTGCCTCTCTGGGTTTGGGAGTGGCTGCTGCCCTGGCCGGGCGCCCTCTATGGGTGGTGGGC
ACTGCACCCTGCCCCTCTGCTCCTTCGGCTGTGGGAGTGGCATCTGCATCGCTCCCAATGTCTGCTCC
TGCCAGGATGGAGAGCAAGGGGCCACCTGCCCAGAAACCCATGGACCATGTGGGGAGTACGGCTGTGA
CCTTACCTGCAACCATGGAGGCTGTCAGGAGGTGGCCCGAGTGTGCCCCGTGGGCTTCTCGATGACGG
AGACAGCTGTTGGCATCAGGTGTACAGACATTGACGAATGTGTAACCTCCTCCTGCGAGGGCCACTGT
GTGAACACAGAAGGTGGGTTTGTGTGCGAGTGTGGGCCGGGCATGCAGCTGTCTGCCGACCGCCACAG
CTGCCAAGACACTGACGAATGCCTAGGGACTCCCTGTCAGCAGAGATGTAAAAACAGCATTGGCAGCT
ACAAGTGCTCCTGTCGAACTGGCTTCCACCTTCATGGCAACCGGCACTCCTGTGTAGATGTAAACGAG
TGTCGGAGGCCATTGGAGAGGCGAGTCTGTCACCATTCCTGCCACAACACCGTGGGCAGCTTCCTATG
CACATGCCGACCTGGCTTCAGGCTCCGAGCTGACCGCGTGTCCTGTGAAGCTTTCCCGAAAGCCGTGC
TGGCCCCATCTGCCATCCTGCAACCCCGGCAACACCCGTCCAAGATGCTTCTGTTGCTTCCTGAGGCC
GGCCGGCCTGCCCTGTCCCCAGGACATAGCCCTCCTTCTGGGGCTCCAGGGCCCCCAGCCGGAGTCAG
AACCACCCGCCTGCCATCTCCCACCCCACGACTACCCACATCCTCCCCTTCTGCCCCTGTGTGGCTGC
TGTCCACCCTGCTGGCCACCCCAGTGCCTACTGCCTCCCTGCTGGGGAACCTCAGACCCCCCTCACTC
CTTCAGGGGGAGGTGATGGGGACCCCTTCCTCACCCAGGGGCCCTGAGTCCCCCCGACTGGCAGCAGG
GCCCTCTCCCTGCTGGCACCTGGGAGCGATGCATGAATCAAGGAGTCGCTGGACAGAGCCTGGGTGTT
CCCAGTGCTGGTGCGAGGACGGGAAGGTGACCTGTGAAAAGGTGAGGTGTGAAGCTGCTTGTTCCCAC
CCAATTCCCTCCAGAGATGGTGGGTGCTGCCCATCGTGCACAGGCTGTTTTCACAGTGGTGTCGTCCG
AGCTGAAGGGGATGTGTTTTCACCTCCCAATGAGAACTGCACCGTCTGTGTCTGTCTGGCTGGAAACG
TGTCCTGCATCTCTCCTGAGGGTCCTTCTGGCCCCTGTCAGACCCCCCCACAGACGGATTGCTGTACT
TGTGTTCCAGTGAGATGCTATTTCCACGGCCGGTGGTACGCAGACGGGGCTGTGTTCAGTGGGGGTGG
TGACGAGTGTACCACCTGTGTTTGCCAGAATGGGGAGGTGGAGTGCTCCTTCATGCCCTGCCCTGAGC
TGGCCTGCCCCCGAGAAGAGTGGCGGCTGGGCCCTGGGCAGTGTTGCTTCACCTGCCAGGAGCCCACA
CCCTCGACAGGCTGCTCTCTTGACGACAACGGGGTTGAGTTTCCGATTGGACAGATCTGGTCGCCTGG
TGACCCCTATGGCTCGGTGAGCTGCAAGAGGACAGACTGTGTGGACTCCTGCCCTCACCCGATCCGGA
TCCCTGGACAGTGCTGCTCGAG

| | | | |
|---|---|---|---|
| NOV59d, 198488424 | SEQ ID NO: 1162 | 596 aa | MW at 62805.5kD |
| Protein Sequence | | | |

GTRGYTGRKPPGHFAAERRRLGPHVCLSGFGSGCCPGHAPSMGGGHCTLPLCSFGCGSGICIAPNVCS
CQDGEQGATCPETHGPCGEYGCDLTCNHGGCQEVARVCPVGFSMTETAVGIRCTDIDECVTSSCEGHC
VNTEGGFVCECGPGMQLSADRHSCQDTDECLGTPCQQRCKNSIGSYKCSCRTGFHLHGNRHSCVDVNE
CRRPLERRVCHHSCHNTVGSFLCTCRPGFRLRADRVSCEAFPKAVLAPSAILQPRQHPSKMLLLLPEA
GRPALSPGHSPPSGAPGPPAGVRTTRLPSPTPRLPTSSPSAPVWLLSTLLATPVPTASLLGNLRPPSL
LQGEVMGTPSSPRGPESPRLAAGPSPCWHLGAMHESRSRWTEPGCSQCWCEDGKVTCEKVRCEAACSH
PIPSRDGGCCPSCTGCFHSGVVPAEGDVFSPPNENCTVCVCLAGNVSCISPEGPSGPCQTPPQTDCCT
CVPVRCYFHGRWYADGAVFSGGGDECTTCVCQNGEVECSFMPCPELACPREEWRLGPGQCCFTCQEPT
PSTGCSLDDNGVEFPIGQIWSPGDPYGSVSCKRTDCVDSCPHPIRIPGQCCS

| | | |
|---|---|---|
| NOV59e, 198488428 | SEQ ID NO: 1163 | 1661 bp |
| DNA Sequence | ORF Start: at 1 | ORF Stop: at 1660 |

GGTACCCGAGGCTACACCGGGAGGAAGCCGCCCGGGCACTTCGCGGCCGAGAGACGCCGACTGGGCCC
CCACGTCTGCCTCTCTGGGTTTGGGAGTGGCTGCTGCCCTGGCTGGGCGCCCTCTATGGGTGGTGGGC
ACTGCACCCTGCCCCTCTGCTCCTTCGGCTGTGGGAGTGGCATCTGCATCGCTCCCAATGTCTGCTCC
TGCCAGGATGGAGAGCAAGGGGCCACCTGCCCAGAAACCCATGGACCATGTGGGGAGTACGGCTGTGA
CCTTACCTGCAACCATGGGGGCTGTCAGGAGGTGGCCCGAGTGTGCCCCGTGGGCTTCTCGATGACGG
AGACAGCTGTTGGCATCAGGTGTACAGACATTGACGAATGTGTAACCTCCTCCTGCGAGGGCCACTGT
GTGAACACAGAAGGTGGGTTTGTGTGCGAGTGTGGGCCGGGCATGCAGCTGTCTGCCGACCGCCACAG
CTGCCAAGACACTGACGAATGCCTAGGGACTCCCTGTCAGCAGAGATGTAAAAACAGCATTGGCAGCT
ACAAGTGTTCCTGTCGAACTGGCTTCCACCTTCATGGCAACCGGCACTCCTGTGTAGCTTCCCCGAAA
GCCGTGCTGGCCCCATCTGCCATCCTGCAACCCCGGCAACACCCGTCCAAGATGCTTCTGTTGCTTCC
TGAGGCCGGCCGGCCTGCCCTGTCCCCAGGACATAGCCCTCCTTCTGGGGCTCCAGGGCCCCCAGCCG
GAGTCAGGACCACCCGCCTGCCATCTCCCACCCCACGACTACCCACATCCTCCCCTTCTGCCCCTGTG
TGGCTGCTGTCCACCCTGCTGGCCACCCCAGTGCCTACTGCCTCCCTGCTGGGGAACCTCAGACCCCC
CTCACTCCTTCAGGGGGAGGTGATGGGGACCCCTTCCTCACCCAGGGGCCCTGAGTCCCCCCGACTGG
CAGCAGGGCCCTCTCCCTGCTGGCACCTGGGAGCCATGCATGAATCAAGGAGTCGCTGGACAGAGCCT
GGGTGTTCCCAGTGCTGGTGCGAGGACGGGAAGGTGACCTGTGAAAAGGTGAGGTGTGAAGCTGCTTG
TTCCCACCCAATTCCCTCCAGAGATGGTGGGTGCTGCCCATCGTGCACAGGCTGTTTTCACAGTGGTG
TCGTCCGAGCTGAAGGGGATGTGTTTTCACCTCCCAATGAGAACTGCACCGTCTGTGTCTGTCTGGCT
GGAAACGTGTCCTGCATCTCTCCTGAGTGTCCTTCTGGCCCCTGTCAGACCCCCCCACAGACGGATTG
CTGTACTTGTGTTCCAGTGAGATGCTATTTCCACGGCCGGTGGTACGCAGACAGGGCTGTGTTCAGTG
GGGGTGGTGACGAGTGTACCACCTGTGTTTGCCAGAATGGGGAGGTGGAGTGCTCCTTCATGCCCTGC
CCTGAGCTGGCCTGCCCCCGAGAAGAGTGGCGGCTGGGCCCTGGGCAGTGTTGCTTCACCTGCCAGGA
GCCCACACCCTCGACAGGCTGCTCTCTTGACGACAACGGGGTTGAGTTTCCGATTGGACAGATCTGGT
CGCCTGGTGACCCCTATGGCTCGGTGAGCTGCAAGAGGACAGACTGTGTGGACTCCTGCCCTCACCCG
ATCCGGATCCCTGGACAGTGCTGCTCGAG

| | | | |
|---|---|---|---|
| NOV59e, 198488428 | SEQ ID NO: 1164 | 553 aa | MW at 57922.0kD |
| Protein Sequence | | | |

GTRGYTGRKPPGHFAAERRRLGPHVCLSGFGSGCCPGWAPSMGGGHCTLPLCSFGCGSGICIAPNVCS
CQDGEQGATCPETHGPCGEYGCDLTCNHGGCQEVARVCPVGFSMTETAVGIRCTDIDECVTSSCEGHC
VNTEGGFVCECGPGMQLSADRHSCQDTDECLGTPCQQRCKNSIGSYKCSCRTGFHLHGNRHSCVASPK
AVLAPSAILQPRQHPSKNLLLLPEAGRPALSPGHSPPSGAPGPPAGVRTTRLPSPTPRLPTSSPSAPV
WLLSTLLATPVPTASLLGNLRPPSLLQGEVMGTPSSPRGPESPRLAAGPSPCWHLGAMHESRSRWTEP
GCSQCWCEDGKVTCEKVRCEAACSHPIPSRDGGCCPSCTGCFHSGVVRAEGDVFSPPNENCTVCVCLA
GNVSCISPECPSGPCQTPPQTDCCTCVPVRCYFHGRWYADRAVFSGGGDECTTCVCQNGEVECSFMPC
PELACPREEWRLGPGQCCFTCQEPTPSTGCSLDDNGVEFPIGQIWSPGDPYGSVSCKRTDCVDSCPHP
IRIPGQCCS

| | | |
|---|---|---|
| NOV59f, 198488440 | SEQ ID NO: 1165 | 1814 bp |
| DNA Sequence | ORF Start: at 1 | ORF Stop: at 1813 |

GGTACCCGAGGCTACACCGGGAAGAAGCCGCCCGGGCACTTCGCGGCCGAGAGACGCCGACTGGGCCC
CCACGTCTGCCTCTCTGGGTTTGGGAGTGGCTGCTGCCCTGGCTGGGCGCCCTCTATGGGTGGTGGGC
ACTGCACCCTGCCCCTCTGCTCCTTCGGCTGTGGGAGTGGCATCTGCATCGCTCCCAATGTCTGCTCC
TGCCAGGATGGAGAGCAAGGGGCCACCTGCCCAGAAACCCATGGACCATGTGGGGAGTACGGCTGTGA
CCTTACCTGCAACCATGGAGGCTGTCAGGAGGTGGCCCGAGTGTGCCCCGTGGGCTTCTCGATGACGG
AGACAGCTGTTGGCATCAGGTGTACAGACATTGACGAATGTGTAACCTCCTCCTGCGAGGGCCACTGT
GTGAACACAGAAGGTGGGTTTGTGTGCGAGTGTGGGCCGGGCATGCAGCTGTCTGCCGACCGCCACAG
CTGCCAAGACACTGACGAATGCCTAGGGACTCCCTGTCAGCAGAGATGTAAAAACAGCATTGGCAGCT
ACAAGTGCTCCTGTCGAACTGGCTTCCACCTTCATGGCAACCGGCACTCCTGTGTAGATGTAAACGAG
TGTCGGAGGCCATTGGAGAGGCGAGTCTGTCACCATTCCTGCCACAACACCGTGGGCAGCTTCCTATG
CACATGCCGACCTGGCTTCAGGCTCCGAGCTGACCGCGTGTCCTGTGAAGCTTTCCCGAAAGCCGTGC
TGGCCCCATCTGCCATCCTGCAACCCCGGCAACACCCGTCCAAGATGCTTCTGTTGCTTCCTGAGGCC
GGCCGGCCTGCCCTGTCCCCAGGACATAGCCCTCCTTCTGGGGCTCCAGGGCCCCCAGCCGGAGTCAG
GACCACCCGCCTGCCATCTCCCACCCCACGACTACCCACATCCTCCCCTTCTGCCCCTGTGTGGCTGC
TGTCCACCCTGCTGGCCACCCCAGTGCCTACTGCCTCCCTGCTGGGGAACCTCAGACCCCCCTCACTC
CTTCAGGGGGAGGTGATGGGGACCCCTTCCTCACCCAGGGGCCCTGAGTCCCCCCGACTGGCAGCAGG
GCCCTCTCCCTGCTGGCACCTGGGAGCGATGCATGAATCAAGGAGTCGCTGGACAGAGCCTGGGTGTT
CCCAGTGCTGGTGCGAGGACGGGAAGGTGACCTGTGAAAAGGTGCGGTGTGAAGCTGCTTGTTCCCAC
CCAATTCCCTCCAGAGATGGTGGGTGCTGCCCATCGTGCACAGGCTGTTTTCACAGTGGTGTCGTCCG
AGCTGAAGGGGATGTGTTTTCACCTCCCAATGAGAACTGCACCGTCTGTGTCTGTCTGGCTGGAAACG
TGTCCTGCATCTCTCCTGAGTGTCCTTCTGGCCCCTGTCAGACCCCCCCACAGACGGATTGCTGTACT
TGTGTTCCAGTGAGATGCTATTTCCACGGCCGGTGGTACGCAGACGGGGCTGTGTTCAGTGGGGGTGG
TGACGAGTGTACCACCTGTGTTTGCCAGAATGGGGAGGTGGAGTGCTCCTTCATGCCCTGCCCTGAGC
TGGCCTGCCCCCGAGAAGAGTGGCGGCCGGGCCCTGGGCAGTGTTGCTTCACCTGCCAGGAGCCCACA
CCCTCGACAGGCTGCTCTCTTGACGACAACGGGGTTGAGTTTCCGATTGGACAGATCTGGTCGCCTGG
TGACCCCTGTGAGTTATGCATCTGCCAGGCAGATGGCTCGGTGAGCTGCAAGAGGACAGACTGTGTGG
ACTCCTGCCCTCACCCGATCCGGATCCCTGGACAGTGCTGCTCGAG

| | | | |
|---|---|---|---|
| NOV59f, 198488440 | SEQ ID NO: 1166 | 604 aa | MW at 63653.5kD |
| Proteing Sequence | | | |

GTRGYTGKKPPGHFAAERRRLGPHVCLSGFGSGCCPGWAPSMGGGHCTLPLCSFGCGSGICIAPNVCS
CQDGEQGATCPETHGPCGEYGCDLTCNHGGCQEVARVCPVGFSMTETAVGIRCTDIDECVTSSCEGHC
VNTEGGFVCECGPGMQLSADRHSCQDTDECLGTPCQQRCKNSIGSYKCSCRTGFHLHGNRHSCVDVNE
CRRPLERRVCHHSCHNTVGSFLCTCRPGFRLRADRVSCEAFPKAVLAPSAILQPRQHPSKMLLLLPEA
GRPALSPGHSPPSGAPGPPAGVRTTRLPSPTPRLPTSSPSAPVWLLSTLLATPVPTASLLGNLRPPSL
LQGEVMGTPSSPRGPESPRLAAGPSPCWHLGAMHESRSRWTEPGCSQCWCEDGKVTCEKVRCEAACSH
PIPSRDGGCCPSCTGCFHSGVVRAEGDVFSPPNENCTVCVCLAGNVSCISPECPSGPCQTPPQTDCCT
CVPVRCYFHGRWYADGAVFSGGGDECTTCVCQNGEVECSFMPCPELACPREEWRPGPGQCCFTCQEPT
PSTGCSLDDNGVEFPIGQIWSPGDPCELCICQADGSVSCKRTDCVDSCPHPIRIPGQCCS

| | | |
|---|---|---|
| NOV59g, CG93871-02 | SEQ ID NO: 1167 | 585 bp |
| DNA Sequence | ORF Start: at 7 | ORF Stop: at 580 |

GGTACCTGCTCCTTCGGCTGTGGGAGTGGCATCTGCATCGCTCCTAATGTCTGCTCCTGCCAGGATGG
AGAGCAAGGGGCCACCTGCCCAGAAACCCATGGACCATGTGGGGAGTACGGCTGTGACCTTACCTGCA
ACCATGGAGGCTGTCAGGAGGTGGCCCGAGTGTGCCCCGTGGGCTTCTCGATGACGGAGACAGCTGTT
GGCATCAGGTGTACAGACATTGACGAATGTGTAACCTCCTCCTGCGAGGGCCACTGTGTGAACACAGA
AGGTGGGTTTGTGTGCGAGTGTGGGCCGGGCATGCAGCTGTCTGCCGACCGCCACAGCTGCCAAGACA
CTGACGAATGCCTAGGGACTCCCTGTCAGCAGGGATGTAAAAACAGCATTGGCAGCTACAAGTGTTCC
TGTCGAACTGGCTTCCACCTTCATGGCAACCGGCACTCCTGTGTAGATGTAAACGAGTGTCGGAGGCC
ATTGGAGAGGCGAGTCTGTCACCATTCCTGCCACAACACCGTGGGCAGCTTCCTATGCACATGCCGAC
CTCGAGACAGGCTCCGAGCTGACCGCGTGTCCTGTCTCGAG

| | | | |
|---|---|---|---|
| NOV59g, CG93871-02 | SEQ ID NO: 1168 | 191 aa | MW at 20429.7kD |
| Protein Sequence | | | |

CSFGCGSGICIAPNVCSCQDGEQGATCPETHGPCGEYGCDLTCNHGGCQEVARVCPVGFSMTETAVGI
RCTDIDECVTSSCEGHCVNTEGGFVCECGPGMQLSADRHSCQDTDECLGTPCQQGCKNSIGSYKCSCR
TGFHLHGNRHSCVDVNECRRPLERRVCHHSCHNTVGSFLCTCRPRDRLRADRVSC

| | | |
|---|---|---|
| NOV59h, CG93871-03 | SEQ ID NO: 1169 | 2063 bp |
| DNA Sequence | ORF Start: ATG at 20 | ORF Stop: TAG at 1229 |

AGTGTTACAGTTCTTCAAGATGGTGTGTCCGGAGTTTGTTCCTTCAGATGTTCAGATGTGTCCAGAAT
TTCTTCCTTCTGGTGGGTTCGTGCTCTCACTGGCTTCAATGGGTGGTGGGCACTGCACCCTGCCCCTC
TGCTCCTTCGGCTGTGGGAGTGGCATCTGCATCGCTCCCAATGTCTGCTCCTGCCAGGATGGAGAGCA
AGGGGCCACCTGCCCAGAAACCCATGGACCATGTGGGGAGTACGGCTGTGACCTTACCTGCAACCATG
GAGGCTGTCAGGAGGTGGCCCGAGTGTGCCCCGTGGGCTTCTCGATGACGGAGACAGCTGTTGGCATC
AGGTGTACAGACATTGACGAATGTGTAACCTCCTCCTGCGAGGGCCACTGTGTGAACACAGAAGGTGG
GTTTGTGTGCGAGTGTGGGCCGGGCATGCAGCTGTCTGCCGACCGCCACAGCTGCCAAGACACTGACG
AATGCCTAGGGACTCCCTGTCAGCAGAGATGTAAAAACAGCATTGGCAGCTACAAGTGTTCCTGTCGA
ACTGGCTTCCACCTTCATGGCAACCGGCACTCCTGTGTAGATGTAAACGAGTGTCGGAGGCCATTGGA
GAGGCGAGTCTGTCACCATTCCTGCCACAACACCGTGGGCAGCTTCCTATGCACATGCCGACCTGGCT
TCAGGCTCCGAGCTGACCGCGTGTCCTGTGAAGCTTTCCCGAAAGCCGTGCTGGCCCCATCTGCCATC
CTGCAACCCCGGCAACACCCGTCCAAGATGCTTCTGTTGCTTCCTGAGGCCGGCCGGCCTGCCCTGTC
CCCAGGACATAGCCCTCCTTCTGGGGCTCCAGGGCCCCCAGCCGGAGTCAGGACCACCCGCCTGCCAT
CTCCCACCCCACGACTACCCACATCCTCCCCTTCTGCCCCTGTGTGGCTGCTGTCCACCCTGCTGGCC
ACCCCAGTGCCTACTGCCTCCCTGCTGGGGAACCTCAGACCCCCCTCACTCCTTCAGGGGGAGGTGAT
GAGGACCCCTTCCTCACCCAGGGGCCCTGAGTCCCCCCGACTGGCAGCAGGGCCCTCTCCCTGCTGGC
ACCTGGGAGCCATGCATGAATCAAGGAGTCGCTGGACAGAGCCTGGGTGTTCCCAGTGCTGGTGCGAG
GTGGGTACTAGGGGTCCCCGGAGCTTCCTTGGTCTGGGGCCTTGTGGTGGCTCTAACTCCTGCTTGTG
CTTCTAGGACGGGAAGGTGACCTGTGAAAAGGTGAGGTGTGAAGCTGCTTGTTCCCACCCAATTCCCT
CCAGAGATGGTGGGTGCTGCCCATCGTGCACAGGTGAGAGCTGGTGCCAGGGAAGGACATAGTCCTGC
CTCTTCCTTTCTCTGCACCCTCCTGCAGCCAAAAGGCTGTTCTTTAACATTAGCATACATTAGAATCT
CCTGGAGGGTTTACTAAGCCACACTTCCCTTGGCGCTGCTCCCAGAGCTTCTGATGTAGTAGGTCTGG
GATAGGGCCAGAACATGTGCGTTTTGAACAGGTTCCCAGGTGATACTGACGTGGCTGGTCTAGGGACC
ATACTTTGAGAACCACTCATTTATGCTGTAATATGCCTCAAGGGGGACTTTTCATCTTCTGGGCATCT
GTTTTCAGCTTCATTCGCTCATTTTCATTTATTCAGTCATTCATTGAGCCTCTTTAAATCCACTGTGC
AGTCCCCTTCTCTTTCCGACTCACCTTCTCACATGGGGAGGAGTAGTTGCTTTACAATTCCCCCCCTT
TTTAATCTCCCCCCCCCCATATATCGATACTTCTTTATCTTCTTCCCATTCCTGGAAGAATAGGGACC
CATATTCTTCACCAAATCCTCAACTAGAGTTTCTTCTGAACTACCTCTCCAGCCCCTCAAGGGTATTC
TGATGACAAATACAAATTCTTCTCGTTGGCACGCTGTCTCTATCGCAGAACCCCACCACTTTGGGAGT
CCAATTTTGTCTTATCACTTGAGCGGACCATTCAGGGACTGTTTGGGATACTCGGTGATAAACGTCGP
GCATTCGTCTTTCTATCTCCTCA

| | | | |
|---|---|---|---|
| NOV59h, CG93871-03 | SEQ ID NO: 1170 | 403 aa | MW at 42493.0kD |
| Protein Sequence | | | |

MVCPEFVPSDVQMCPEFLPSGGFVLSLASMGGGHCTLPLCSFGCGSGICIAPNVCSCQDGEQGATCPE
THGPCGEYGCDLTCNHGGCQEVARVCPVGFSMTETAVGIRCTDIDECVTSSCEGHCVNTEGGFVCECG
PGMQLSADRHSCQDTDECLGTPCQQRCKNSIGSYKCSCRTGFHLHGNRHSCVDVNECRRPLERRVCHH
SCHNTVGSFLCTCRPGFRLRADRVSCEAFPKAVLAPSAILQPRQHPSKMLLLLPEAGRPALSPGHSPP
SGAPGPPAGVRTTRLPSPTPRLPTSSPSAPVWLLSTLLATPVPTASLLGNLRPPSLLQGEVMRTPSSP
RGPESPRLAAGPSPCWHLGAMHESRSRWTEPGCSQCWCEVGTRGPRSFLGLGPCGGSNSCLCF

| | | |
|---|---|---|
| NOV59i, CG93871-04 | SEQ ID NO: 1171 | 1790 bp |
| DNA Sequence | ORF Start: at 1 | ORF Stop: at 1789 |

GGTACCCGAGGCTACACCGGGAGGAAGCCGCCCGGGCACTTCGCGGCCGAGAGACGCCGACTGGGCCC
CCACGTCTGCCTCTCTGGGTTTGGGAGTGGCTGCTGCCCTGGCCGGGCGCCCTCTATGGGTGGTGGGC
ACTGCACCCTGCCCCTCTGCTCCTTCGGCTGTGGGAGTGGCATCTGCATCGCTCCCAATGTCTGCTCC
TGCCAGGATGGAGAGCAAGGGGCCACCTGCCCAGAAACCCATGGACCATGTGGGGAGTACGGCTGTGA
CCTTACCTGCAACCATGGAGGCTGTCAGGAGGTGGCCCGAGTGTGCCCCGTGGGCTTCTCGATGACGG
AGACAGCTGTTGGCATCAGGTGTACAGACATTGACGAATGTGTAACCTCCTCCTGCGAGGGCCACTGT
GTGAACACAGAAGGTGGGTTTGTGTGCGAGTGTGGGCCGGGCATGCAGCTGTCTGCCGACCGCCACAG
CTGCCAAGACACTGACGAATGCCTAGGGACTCCCTGTCAGCAGAGATGTAAAAACAGCATTGGCAGCT
ACAAGTGCTCCTGTCGAACTGGCTTCCACCTTCATGGCAACCGGCACTCCTGTGTAGATGTAAACGAG
TGTCGGAGGCCATTGGAGAGGCGAGTCTGTCACCATTCCTGCCACAACACCGTGGGCAGCTTCCTATG
CACATGCCGACCTGGCTTCAGGCTCCGAGCTGACCGCGTGTCCTGTGAAGCTTTCCCGAAAGCCGTGC
TGGCCCCATCTGCCATCCTGCAACCCCGGCAACACCCGTCCAAGATGCTTCTGTTGCTTCCTGAGGCC
GGCCGGCCTGCCCTGTCCCCAGGACATAGCCCTCCTTCTGGGGCTCCAGGGCCCCCAGCCGGAGTCAG
AACCACCCGCCTGCCATCTCCCACCCCACGACTACCCACATCCTCCCCTTCTGCCCCTGTGTGGCTGC
TGTCCACCCTGCTGGCCACCCCAGTGCCTACTGCCTCCCTGCTGGGGAACCTCAGACCCCCCTCACTC
CTTCAGGGGGAGGTGATGGGGACCCCTTCCTCACCCAGGGGCCCTGAGTCCCCCCGACTGGCAGCAGG
GCCCTCTCCCTGCTGGCACCTGGGAGCGATGCATGAATCAAGGAGTCGCTGGACAGAGCCTGGGTGTT
CCCAGTGCTGGTGCGAGGACGGGAAGGTGACCTGTGAAAAGGTGAGGTGTGAAGCTGCTTGTTCCCAC
CCAATTCCCTCCAGAGATGGTGGGTGCTGCCCATCGTGCACAGGCTGTTTTCACAGTGGTGTCGTCCG
AGCTGAAGGGGATGTGTTTTCACCTCCCAATGAGAACTGCACCGTCTGTGTCTGTCTGGCTGGAAACG
TGTCCTGCATCTCTCCTGAGGGTCCTTCTGGCCCCTGTCAGACCCCCCCACAGACGGATTGCTGTACT
TGTGTTCCAGTGAGATGCTATTTCCACGGCCGGTGGTACGCAGACGGGGCTGTGTTCAGTGGGGGTGG
TGACGAGTGTACCACCTGTGTTTGCCAGAATGGGGAGGTGGAGTGCTCCTTCATGCCCTGCCCTGAGC
TGGCCTGCCCCCGAGAAGAGTGGCGGCTGGGCCCTGGGCAGTGTTGCTTCACCTGCCAGGAGCCCACA
CCCTCGACAGGCTGCTCTCTTGACGACAACGGGGTTGAGTTTCCGATTGGACAGATCTGGTCGCCTGG
TGACCCCTATGGCTCGGTGAGCTGCAAGAGGACAGACTGTGTGGACTCCTGCCCTCACCCGATCCGGA
TCCCTGGACAGTGCTGCTCGAG

| | | | |
|---|---|---|---|
| NOV59i, CG93871-04 | SEQ ID NO: 1172 | 596 aa | MW at 62805.5kD |
| Protein Sequence | | | |

GTRGYTGRKPPGHFAAERRRLGPHVCLSGFGSGCCPGRAPSMGGGHCTLPLCSFGCGSGICIAPNVCS
CQDGEQGATCPETHGPCGEYGCDLTCNHGGCQEVARVCPVGFSMTETAVGIRCTDIDECVTSSCEGHC
VNTEGGFVCECGPGMQLSADRHSCQDTDECLGTPCQQRCKNSIGSYKCSCRTGFHLHGNRHSCVDVNE
CRRPLERRVCHHSCHNTVGSFLCTCRPGFRLRADRVSCEAFPKAVLAPSAILQPRQHPSKMLLLLPEA
GRPALSPGHSPPSGAPGPPAGVRTTRLPSPTPRLPTSSPSAPVWLLSTLLATPVPTASLLGNLRPPSL
LQGEVMGTPSSPRGPESPRLAAGPSPCWHLGAMHESRSRWTEPGCSQCWCEDGKVTCEKVRCEAACSH
PIPSRDGGCCPSCTGCFHSGVVRAEGDVFSPPNENCTVCVCLAGNVSCISPEGPSGPCQTPPQTDCCT
CVPVRCYFHGRWYADGAVFSGGGDECTTCVCQNGEVECSFMPCPELACPREEWRLGPGQCCFTCQEPT
PSTGCSLDDNGVEFPIGQIWSPGDPYGSVSCKRTDCVDSCPHPIRIPGQCCS

A ClustalW comparison of the above protein sequences yields the following sequence alignment shown in Table 59B.

TABLE 59B
Comparison of the NOV59 protein sequences.

NOV59a MWAGLLLRAACVALLLPGAPARGYTGRKPPGHFAAERRRLGPNVCLSGFGSGCCPGWAPS
NOV59b MWAGLLLRAACVALLLPGAPARGYTGRKPPGHFAAERRRLGPHVCLSGFGSGCCPGWAPS
NOV59c -------------------GTRGYTGRKPPGHFAAERRRLGPHVCLSGFGSGCCPGWAPS
NOV59d -------------------GTRGYTGRKPPGHFAAERRRLGPHVCLSGFGSGCCPGRAPS
NOV59e -------------------GTRGYTGRKPPGHFAAERRRLGPHVCLSGFGSGCCPGWAPS
NOV59f -------------------GTRGYTGKKPPGHFAAERRRLGPHVCLSGFGSGCCPGWAPS
NOV59g ------------------------------------------------------------
NOV59h -------------MVCP--------------EFVPS----DVQMCPEFLPSGGFVLSLAS
NOV59i -------------------GTRGYTGRKPPGHFAAERPRLGPHVCLSGFGSGCCPGRAPS
NOV59a MGGGHCTLRLCSFGCGSGICIAPNVCSCQDGEQGATCPETHGPCGEYGCDLTCNHGGCQE
NOV59b MGGGHCTLPLCSLGCGSGICIAPNVCSCQDGEQGATCPETHGPCGEYGCDLTCNHGGCQE
NOV59c MGGGHCTLPPCSFGCGSGICIAPNVCSCQDGEQGATCPETHGPCGEYGCDLTCNHGGCQE
NOV59d MGGGHCTLPLCSFGCGSGICIAPNVCSCQDGEQGATCPETHGPCGEYGCDLTCNHGGCQE
NOV59e MGGGHCTLPLCSFGCGSGICIAPNVCSCQDGEQGATCPETHGPCGEYGCDLTCNHGGCQE
NOV59f MGGGHCTLPLCSFGCGSGICIAPNVCSCQDGEQGATCPETHGPCGEYGCDLTCNHGGCQE
NOV59g ----------CSFGCGSGICIAPNVCSCQDGEQGATCPETHGPCGEYGCDLTCNHGGCQE
NOV59h MGGGHCTLPLCSFGCGSGICIAPNVCSCQDGEQGATCPETHGPCGEYGCDLTCNHGGCQE
NOV59i MGGGHCTLPLCSFGCGSGICIAPNVCSCQDGEQGATCPETHGPCGEYGCDLTCNHGGCQE
NOV59a VARVCPVGFSMTETAVGIRC-DIDECVTSSCEGHCVNTEGGFVCECGPGMQLSADRHSCQ
NOV59b VARVCPVGFSMTETAVGIRCTDIDECVTSSCEGHCVNTE---------------------
NOV59c VARVCPVGFSMTETAVGIRCTDIDECVTSSCEGHCVNTESGFVCECGPGMQLSADRHSCQ
NOV59d VARVCPVGFSMTETAVGIRCTDIDECVTSSCEGHCVNTEGGFVCECGPGMQLSADRHSCQ
NOV59e VARVCPVGFSMTETAVGIRCTDIDECVTSSCEGHCVNTEGGFVCECGPGMQLSADRHSCQ
NOV59f VARVCPVGFSMTETAVGIRCTDIDECVTSSCEGHCVNTEGGFVCECGPGMQLSADRHSCQ
NOV59g VARVCPVGFSMTETAVGIRCTDIDECVTSSCEGHCVNTEGGFVCECGPGMQLSADRHSCQ
NOV59h VARVCPVGFSMTETAVGIRCTDIDECVTSSCEGHCVNTEGGFVCECGPGMQLSADRHSCQ
NOV59i VARVCPVGFSMTETAVGIRCTDIDECVTSSCEGHCVNTEGGFVCECGPGMQLSADRHSCQ
NOV59a DTDECLGTPCQQRCKNSIGSYKCSCRTGFHLHGNRHSCVDVNECRRPLERRVCHHSCHNT
NOV59b DTDECLGTPCQQRCKNSIGSYKCSCRTGFHLHGNRHSCVDVNECRRPLERRVCHHSCHNT
NOV59c DTDECLGTPCQQRCKNSIGSYKCSCRTGFHLHGNRHSCVDVNECRRPLERRVCHHSCHNT
NOV59d DTDECLGTPCQQRCKNSIGSYKCSCRTGFHLHGNRHSCVDVNECRRPLERRVCHHSCHNT
NOV59e DTDECLGTPCQQRCKNSIGSYKCSCRTGFHLHGNRHSCVAS-------------------
NOV59f DTDECLGTPCQQRCKNSIGSYKCSCRTGFHLHGNRHSCVDVNECRRPLERRVCHHSCHNT
NOV59g DTDECLGTPCQQGCKNSIGSYKCSCRTGFHLHGNRHSCVDVNECRRPLERRVCHHSCHNT
NOV59h DTDECLGTPCQQRCKNSIGSYKCSCRTGFHLHGNRHSCVDVNECRRPLERRVCHHSCHNT
NOV59i DTDECLGTPCQQRCKNSIGSYKCSCRTGFHLHGNRHSCVDVNECRRPLERRVCHHSCHNT
NOV59a VGSFLCTCRPGFRLRADRVSCEAFPKAVLAPSAILQPRQHPSKMLLLLPEAGRPALSPGH
NOV59b VGSFLCTCRPGFRLRADRVSCEAFPKAVLAPSAILQPRQHPSKMLLLLPEAGRPALSPGH
NOV59c VGSFLCTCRPGFRLEADRVSCEAFPKAVLAPSAILQPRQHPSKMLLLLPEAGRPALSPGH
NOV59d VGSFLCTCRPGFRLRADRVSCEAFPKAVLAPSAILQPRQHPSKMLLLLPEAGRPALSPGH
NOV59e ------------------------PKAVLAPSAILQPRQHPSKMLLLLPEAGRPALSPGH
NOV59f VGSFLCTCRPGFRLRADRVSCEAFPKAVLAPSAILQPRQHPSKMLLLLPEAGRPALSPGH
NOV59g VGSFLCTCRPRDRLRADRVSC---------------------------------------
NOV59h VGSFLCTCRPGFRLRADRVSCEAFPKAVLAPSAILQPRQHPSKMLLLLPEAGRPALSPGH
NOV59i VGSFLCTCRPGFRLRADRVSCEAFPKAVLAPSAILQPRQHPSKMLLLLPEAGRPALSPGH
NOV59a SPPSGAPGPPAGVRTTRLPSPTPRLPTSSPSAPVWLLSTLLATPVPTASLLGNLRPPSLL
NOV59b SPPSGAPGPPAGVRTTRLPSPTPRLPTSSPSAPVWLLSTLLATPVPTASLLGNLRPPSLL
NOV59c SPPSGAPGPPAGVRTTRLPSPTPRLPTSSPSAPVWLLSTLLATPVPTASLLGNLRPPSLL
NOV59d SPPSGAPGPPAGVRTTRLPSPTPRLPTSSPSAPVWLLSTLLATPVPTASLLGNLRPPSLL
NOV59e SPPSGAPGPPAGVRTTRLPSPTPRLPTSSPSAPVWLLSTLLATPVPTASLLGNLRPPSLL
NOV59f SPPSGAPGPPAGVRTTRLPSPTPRLPTSSPSAPVWLLSTLLATPVPTASLLGNLRPPSLL
NOV59g ------------------------------------------------------------
NOV59h SPPSGAPGPPAGVRTTRLPSPTPRLPTSSPSAPVWLLSTLLATPVPTASLLGNLRPPSLL
NOV59i SPPSGAPGPPAGVRTTRLPSPTPRLPTSSPSAPVWLLSTLLATPVPTASLLGNLRPPSLL
NOV59a QGEVMGTPSSPRGPESPRLAAGPSPCWHLGAMHESRSRWTEPGCSQCWCEDGKVTCEKVR
NOV59b QGEVMGTPSSPRGPESPRLAAGPSPCWHLGAMHESRSRWTEPGCSQCWCEDGKVTCEKVR
NOV59c QGEVMGTPSSPRGPESPRLAAGPSPCWHLGAMYESRSRWTEPGCSQCWCEDGKVTCEKVR
NOV59d QGEVMGTPSSPRGPESPRIAAGPSPCWHLGAMHESRSRWTEPGCSQCWCEDGKVTCEKVR
NOV59e QGEVMGTPSSPRGPESPRLAAGPSPCWHLGAMHESRSRWTEPGCSQCWCEDGKVTCEKVR
NOV59f QGEVMGTPSSPRGPESPRLAAGPSPCWHLGAMHESRSRWTEPGCSQCWCEDGKVTCEKVR
NOV59g ------------------------------------------------------------
NOV59h QGEVMRTPSSPRGPESPRLAAGPSPCWHLGAMHESRSRWTEPGCSQCWCEVG--------
NOV59i QGEVMGTPSSPRGPESPRLAAGPSPCWHLGAMHESRSRWTEPGCSQCWCEDGKVTCEKVR
NOV59a CEAACSHPIPSRDGGCCPSCTGCFHSGVVRAEGDVFSPPNENCTVCVCLAGNVSCMFREC
NOV59b CEAACSHPIPSRDGGCCPSCTGCFHSGVVPAEGDVFSPPNENCTVCVCLAGNVSCISPEC
NOV59c CEAACSHPIPSRDGGCCPSCTGCFHSGVVPAEGDVFSPPNENCTVCVCLAGNVSCISPEC
NOV59d CEAACSHPIPSRDGGCCPSCTGCFHSGVVRAEGDVFSPPNENCTVCVCLAGNVSCISPEG
NOV59e CEAACSHPIPSRDGGCCPSCTGCFHSGVVRAEGDVFSPPNENCTVCVCLAGNVSCISPEC
NOV59f CEAACSHPIPSRDGGCCPSCTGCFHSGVVRAEGDVFSPPNENCTVCVCLAGNVSCISPEC
NOV59g ------------------------------------------------------------
NOV59h ----------------------------TRGPRSFLG-----------------------
NOV59i CEAACSHPIPSRDGGCCPSCTGCFHSGVVRAEGDVFSPPNENCTVCVCLAGNVSCISPEG
NOV59a PFGPCETPHK--D------RCYFHGRWYADGAVFSGGGDECTTCVCQNGEVECSFMPCPE
NOV59b PSGPCQTPPQTDCCTCVPVRCYFHGRWYADGAVFSGGGDECTTCVCQNGEVECSFMPCPE
NOV59c PSGPCQTPPQTDCCTCVPVRCYFHGRWYADGAVFSGGGDECTTCVCQNGEVECSFMPCPE
NOV59d PSGPCQTPPQTDCCTCVPVRCYFHGRWYADGAVFSGGGDECTTCVCQNGEVECSFMPCPE
NOV59e PSGPCQTPPQTDCCTCVPVRCYFHGRWYADRAVFSGGGDECTTCVCQNGEVECSFMPCPE
NOV59f PSGPCQTPPQTDCCTCVPVRCYFHGRWYADGAVFSGGGDECTTCVCQNGEVECSFMPCPE
NOV59g ------------------------------------------------------------
NOV59h -LGPCGGSNS--C------LCF--------------------------------------
NOV59i PSGPCQTPPQTDCCTCVPVRCYFHGRWYADGAVFSGGGDECTTCVCQNGEVECSFMPCPE
NOV59a LACPREEWRLGPGQCCFTCQEPTPSTGCSLDDNGVEFPIGQIWSPGDPCR-------WLG
NOV59b LACPREEWRLGPGQCCFTCQEPTPSTGCSLDDNGVEFPIGQIWSPGDPCELCICQADGSV
NOV59c LACPREEWRLGPGQCCFTCQEPTPSTGCSLDDNGVEFPIGQTWSPGDPCELCICQADGSV
NOV59d LACPREEWRLGPGQCCFTCQEPTPSTGCSLDDNGVEFPIGQTWSPGDP--------YGSV
NOV59e LACPREEWRLGPGQCCFTCQEPTPSTGCSLDDNGVEFPIGQIWSPGD--------PYGSV
NOV59f LACPREEWRPGPGQCCFTCQEPTPSTGCSLDDNGVEFPIGQIWSPGDPCELCICQADGSV
NOV59g ------------------------------------------------------------
NOV59h ------------------------------------------------------------
NOV59i LACPREEWRLGPGQCCFTCQEPTPSTGCSLDDNGVEFPIGQIWSPGDP--------YGSV
NOV59a ELQEDRLCGLLPSPDPDPWTVLPRLFSR--------------------------------
NOV59b SCKRTDCVDSCPHPIRIPGQCCPDCSAGCTYTGRIFYNNETFPSVLDPCLSCICLLGSVA
NOV59c SCKRTDCVDSCPHPIRIPGQCCS-------------------------------------
NOV59d SCKRTDCVDSCPHPIRIPGQCCS-------------------------------------
NOV59e SCKRTDCVDSCPHPIRIPGQCCS-------------------------------------
NOV59f SCKRTDCVDSCPHPIRIPGQCCS-------------------------------------
NOV59g ------------------------------------------------------------
NOV59h ------------------------------------------------------------
NOV59i SCKRTDCVDSCPHPIRIPGQCCS-------------------------------------
NOV59a ------------------------------------------------------------
NOV59b CSPVDCPITCTYPFHPDGECCPVCRDCNYEGRKVANGQVFTLDDEPCTRCTCQLGEVSCE
NOV59c ------------------------------------------------------------
NOV59d ------------------------------------------------------------
NOV59e ------------------------------------------------------------
NOV59f ------------------------------------------------------------
NOV59g ------------------------------------------------------------
NOV59h ------------------------------------------------------------
NOV59i ------------------------------------------------------------
NOV59a ------------------------------------------------------------
NOV59b KVPCQRACADPALLPGDCCSSCPDSLSPLEEKQGLSPHGNVAFSKAGRSLHGDTEAPVNC
NOV59c ------------------------------------------------------------
NOV59d ------------------------------------------------------------
NOV59e ------------------------------------------------------------
NOV59f ------------------------------------------------------------
NOV59g ------------------------------------------------------------
NOV59h ------------------------------------------------------------
NOV59i ------------------------------------------------------------
NOV59a ------------------------------------------------------------
NOV59b SSCPGPPTASPSRPVLHLLQLLLRTNLMKTQTLPTSPAGAHGPHSLALGLTATFPGEPGA
NOV59c ------------------------------------------------------------
NOV59d ------------------------------------------------------------
NOV59e ------------------------------------------------------------
NOV59f ------------------------------------------------------------
NOV59g ------------------------------------------------------------
NOV59h ------------------------------------------------------------
NOV59i ------------------------------------------------------------
NOV59a ------------------------------------------------------------
N0V59b SPRLSPGPSTPPGAPTLPLASPGAPQPPPVTPERSFSASGAQIVSRWPPLPGTLLTEASA
NOV59c ------------------------------------------------------------
NOV59d ------------------------------------------------------------
NOV59e ------------------------------------------------------------
NOV59f ------------------------------------------------------------
NOV59g ------------------------------------------------------------
NOV59h ------------------------------------------------------------
NOV59i ------------------------------------------------------------
NOV59a -------------------------------------------------------
NOV59b LSMMDPSPSKTPITLLGPRVLSPTTSRLSTALAATTHPGPQQPPVGASRGEESTM
NOV59c -------------------------------------------------------
NOV59d -------------------------------------------------------
NOV59e -------------------------------------------------------
NOV59f -------------------------------------------------------
NOV59g -------------------------------------------------------
NOV59h -------------------------------------------------------
NOV59i -------------------------------------------------------
NOV59a (SEQ ID NO: 1156)
NOV59b (SEQ ID NO: 1158)
NOV59c (SEQ ID NO: 1160)
NOV59d (SEQ ID NO: 1162)
NOV59e (SEQ ID NO: 1164)
NOV59f (SEQ ID NO: 1166)
NOV59g (SEQ ID NO: 1168)
NOV59h (SEQ ID NO: 1170)
NOV59i (SEQ ID NO: 1172)

Further analysis of the NOV59a protein yielded the following properties shown in Table 59C.

TABLE 59C
Protein Sequence Properties NOV59a

| | |
|---|---|
| SignalP analysis: | Cleavage site between residues 22 and 23 |
| PSORT II analysis: | |

| PSG: | a new signal peptide prediction method | |
|---|---|---|
| | N-region: | length 8; pos. chg 1; neg. chg 0 |
| | H-region: | length 13; peak value 9.31 |
| | PSG score: | 4.91 |

| | |
|---|---|
| GvH: | von Heijne's method for signal seq. recognition |
| | GvH score (threshold: −2.1): 1.18 |
| | possible cleavage site: between 21 and 22 |

>>> Seems to have a cleavable signal peptide (1 to 21)

| ALOM: | Klein et al's method for TM region allocation | |
|---|---|---|
| | Init position for calculation: 22 | |
| | Tentative number of TMS(s) for the threshold 0.5: 0 | |
| | number of TMS(s) . . . fixed | |
| | PERIPHERAL | Likelihood = 1.70 (at 70) |

| | |
|---|---|
| | ALOM score: 1.70 (number of TMSs: 0) |
| MTOP: | Prediction of membrane topology (Hartmann et al.) |
| | Center position for calculation: 10 |
| | Charge difference: 0.5 C(2.5)-N(2.0) |
| | C > N: C-terminal side will be inside |

>>>Caution: Inconsistent mtop result with signal peptide

| MITDISC: | discrimination of mitochondrial targeting seq | | | |
|---|---|---|---|---|
| | R content: | 3 | Hyd Moment(75): | 9.30 |
| | Hyd Moment(95): | 7.43 | G content: | 5 |
| | D/E content: | 1 | S/T content: | 1 |

| | |
|---|---|
| | Score: −3.74 |
| Gavel: | prediction of cleavage sites for mitochondrial preseq |
| | R-2 motif at 79 LRL\|CS |

NUCDISC: discrimination of nuclear localization signals

| | |
|---|---|
| | pat4: none |
| | pat7: none |
| | bipartite: none |
| | content of basic residues: 8.3% |
| | NLS Score: −0.47 |
| KDEL: | ER retention motif in the C-terminus: none |

ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear |
| | discrimination |
| | Prediction: nuclear |
| | Reliability: 89 |
| COIL: | Lupas's algorithm to detect coiled-coil regions |
| | total: 0 residues |

Final Results (k = 9/23):

33.3%: extracellular, including cell wall
33.3%: mitochondrial
33.3%: nuclear

>> prediction for CG93871-01 is exc (k = 9)

A search of the NOV59a protein against the Geneseq database, a proprietary database that contains sequences published in patents and patent publication, yielded several homologous proteins shown in Table 59D.

TABLE 59D
Geneseq Results for NOV59a

| | | NOV59a | Identities/ | | |
|---|---|---|---|---|---|
| | | Residues/ | Similarities for | | |
| Geneseq | Protein/Organism/Length | Match | the Matched | | Expect |
| Identifier | [Patent #,Date] | Residues | Region | | Value |
| ABU12085 | Human NOV26a CG93871-01 protein | 1 . . . 612 | 612/612 | (100%) | 0.0 |
| | SEQ ID 90 - Homo sapiens, 612 aa. | 1 . . . 612 | 612/612 | (100%) | |
| | [WO200281625-A2, 17-OCT-2002] | | | | |
| ABU11798 | Human MDDT polypeptide SEQ ID | 381 . . . 580 | 192/208 | (92%) | e−126 |
| | 745 - Homo sapiens, 514 aa. | 1 . . . 208 | 195/208 | (93%) | |
| | [WO200279449-A2, 10-OCT-2002] | | | | |
| AAB85364 | Novel Von Willebrand/thrombosporin- | 283 . . . 490 | 201/208 | (96%) | e−124 |
| | like polypeptide - Homo sapiens, 235 | 1 . . . 208 | 203/208 | (96%) | |
| | aa. [WO200153485-A1, 26-JUL-2001] | | | | |
| AAM99920 | Human polypeptide SEQ ID NO 36 - | 388 . . . 580 | 185/201 | (92%) | e−119 |
| | Homo sapiens, 272 aa. | 5 . . . 205 | 188/201 | (93%) | |
| | [WO200155173-A2, 02-AUG-2001] | | | | |
| AAM99933 | Human polypeptide SEQ ID NO 49 - | 388 . . . 580 | 181/201 | (90%) | e−117 |
| | Homo sapiens, 212 aa. | 5 . . . 205 | 185/201 | (91%) | |
| | [WO200155173-A2, 02-AUG-2001] | | | | |

In a BLAST search of public sequence databases, the NOV59a protein was found to have homology to the proteins shown in the BLASTP data in Table 59E.

TABLE 59E
Public BLASTP Results for NOV59a

| | | NOV59a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96DN2 | Hypothetical protein FLJ32009 - | 1 . . . 580 | 570/589 (96%) | 0.0 |
| | Homo sapiens (Human), 955 aa. | 1 . . . 589 | 573/589 (96%) | |
| AAH43473 | 1300015B04Rik protein - Mus | 1 . . . 606 | 507/615 (82%) | 0.0 |
| | musculus (Mouse), 608 aa. | 1 . . . 607 | 538/615 (87%) | |
| AAO47606 | URG11 - Homo sapiens (Human), | 283 . . . 580 | 290/306 (94%) | 0.0 |
| | 673 aa. | 1 . . . 306 | 293/306 (94%) | |
| Q8IVT0 | Similar to hypothetical protein | 1 . . . 218 | 216/219 (98%) | e−137 |
| | FLJ32009 - Homo sapiens (Human), | 19 . . . 237 | 217/219 (98%) | |
| | 237 aa (fragment). | | | |
| Q8IXK1 | C1q receptor protein precursor - | 80 . . . 375 | 104/300 (34%) | 6e−32 |
| | Homo sapiens (Human), 652 aa. | 300 . . . 566 | 134/300 (44%) | |

PFam analysis predicts that the NOV59a protein contains the domains shown in the Table 59F.

TABLE 59F
Domain Analysis of NOV59a

| | | Identities/ | |
|---|---|---|---|
| Pfam | NOV59a | Similarities for | Expect |
| Domain | Match Region | the Matched Region | Value |
| EGF | 145 . . . 178 | 16/47 (34%) | 0.0047 |
| | | 23/47 (49%) | |
| EGF | 184 . . . 217 | 12/47 (26%) | 0.012 |
| | | 25/47 (53%) | |
| TIL | 165 . . . 223 | 13/70 (19%) | 0.53 |
| | | 40/70 (57%) | |
| EGF | 223 . . . 260 | 12/48 (25%) | 0.045 |
| | | 26/48 (54%) | |
| vwc | 385 . . . 439 | 21/84 (25%) | 7.8e−08 |
| | | 40/84 (48%) | |
| vwc | 442 . . . 492 | 18/84 (21%) | 0.00017 |
| | | 39/84 (46%) | |
| vwc | 493 . . . 550 | 21/84 (25%) | 1.8e−07 |
| | | 40/84 (48%) | |

### Example 60

The NOV60 clone was analyzed, and the nucleotide and encoded polypeptide sequences are shown in Table 60A.

TABLE 60A

| NOV60 Sequence Analysis | | |
|---|---|---|
| NOV60a, CG94946-01 | SEQ ID NO: 1173 | 6224 bp |
| DNA Sequence | ORF Start: ATG at 37 | ORF Stop: TGA at 6196 |

CCGGCGCGGCCCGCGCGCTCTTCCGCCGCCTCTCGCATGCGCCATGGCCGGCCGGTCCCACCCGGGCC
CGCTGCGGGGCGGCCGCTGCTGCCTCTCCTTGTGGTGGCCGCGTGCGTCCTGCCCGGAGCCGGCGGGA
CATGCCCGGAGCGCGCGCTGGAGCGGCGCGAGGAGGAGGCGAACGTGGTGCTCACCGGGACGGTGGAG
GAGATCCTCAACGTGGACCCGGTGCAGCACACGTACTCCTGCAAGGTTCGGGTCTGGCGGTACTTGAA
GGGCAAAGACCTGGTGGCCCGGGAGAGCCTGCTGGACGGCGGCAACAAGGTGGTGATCAGCGGCTTTG
GAGACCCCCTCATCTGTGACAACCAGGTGTCCACTGGGGACACCAGGATCTTCTTTGTGAACCCTGCA
CCCCCATACCTGTGGCCAGCCCACAAGAACGAGCTGATGCTCAACTCCAGCCTCATGCGGATCACCCT
GCGGAACCTGGAGGAGGTGGAGTTCTGTGTGGAAGATAAACCCGGGACCCACTTCACTCCAGTGCCTC
CGACGCCTCCTGATGCGTGCCGGGGAATGCTGTGCGGCTTCGGCGCCGTGTGCGAGCCCAACGCGGAG
GGGCCGGGCCGGGCGTCCTGCGTCTGCAAGAAGAGCCCGTGCCCCAGCGTGGTGGCGCCTGTGTGTGG
GTCGGACGCCTCCACCTACAGCAACGAATGCGAGCTGCAGCGGGCGCAGTGCAGCCAGCAGCGCCGCA
TCCGCCTGCTCAGCCGCGGGCCGTGCGGCTCGCGGGACCCCTGCTCCAACGTGACCTGCAGCTTCGGC
AGCACCTGTGCGCGCTCGGCCGACGGGCTGACGGCCTCGTGCCTGTGCCCCGCGACCTGCCGTGGCGC
CCCCGAGGGGACCGTCTGCGGCAGCGACGGCGCCGACTACCCCGGCGAGTGCCAGCTCCTGCGCCGCG
CCTGCGCCCGCCAGGAGAATGTCTTCAAGAAGTTCGACGGCCCTTGTGACCCCTGTCAGGGCGCCCTC
CCTGACCCGAGCCGCAGCTGCCGTGTGAACCCGCGCACGCGGCGCCCTGAGATGCTCCTACGGCCCGA
GAGCTGCCCTGCCCGGCAGGCGCCAGTGTGTGGGGACGACGGAGTCACCTACGAAAACGACTGTGTCA
TGGGCCGATCGGGGGCCGCCCGGGGTCTCCTCCTGCAGAAAGTGCGCTCCGGCCAGTGCCAGGGTCGA
GACCAGTGCCCGGAGCCCTGCCGGTTCAATGCCGTGTGCCTGTCCCGCCGTGGCCGTCCCCGCTGCTC
CTGCGACCGCGTCACCTGTGACGGGGCCTACAGGCCCGTGTGTGCCCAGGACGGGCGCACGTATGACA
GTGATTGCTGGCGGCAGCAGGCTGAGTGCCGGCAGCAGCGTGCCATCCCCAGCAAGCACCAGGGCCCG
TGTGACCAGGCCCCGTCCCCATGCCTCGGGGTGCAGTGTGCATTTGGGGCGACGTGTGCTGTGAAGAA
CGGGCAGGCAGCGTGTGAATGCCTGCAGGCGTGCTCGAGCCTCTACGATCCTGTGTGCGGCAGCGACG
GCGTCACATACGGCAGCGCGTGCGAGCTGGAGGCCACGGCCTGTACCCTCGGGCGGGAGATCCAGGTG
GCGCGCAAAGGACCCTGTGACCGCTGCGGGCAGTGCCGCTTTGGAGCCCTGTGCGAGGCCGAGACCGG
GCGCTGCGTGTGCCCCTCTGAATGCGTGGCTTTGGCCCAGCCCGTGTGTGGCTCCGACGGGCACACGT
ACCCCAGCGAGTGCATGCTGCACGTGCACGCCTGCACACACCAGATCAGCCTGCACGTGGCCTCAGCT
GGACCCTGTGAGACCTGTGGAGATGCCGTGTGTGCTTTTGGGGCTGTGTGCTCCGCAGGGCAGTGTGT
GTGTCCCCGGTGTGAGCACCCCCCGCCCGGCCCCGTGTGTGGCAGCGACGGTGTCACCTACGGCAGTG
CCTGCGAGCTACGGGAAGCCGCCTGCCTCCAGCAGACACAGATCGAGGAGGCCCGGGCAGGGCCGTGC
GAGCAGGCCGAGTGCGGTTCCGGAGGCTCTGGCTCTGGGGAGGACGGTGACTGTGAGCAGGAGCTGTG
CCGGCAGCGCGGTGGCATCTGGGACGAGGACTCGGAGGACGGGCCGTGTGTCTGTGACTTCAGCTGCC
AGAGTGTCCCAGGCAGCCCGGTGTGCGGCTCAGATGGGGTCACCTACAGCACCGAGTGTGAGCTGAAG
AAGGCCAGGTGTGAGTCACAGCGAGGGCTCTACGTAGCGGCCCAGGGAGCCTGCCGAGGCCCCGCCTT
CGCCCCGCTGCCGCCTGTGGCCCCCTTACACTGTGCCCAGACGCCCTACGGCTGCTGCCAGGACAATA
TCACCGCAGCCCGGGGCGTGGGCCTGGCTGGCTGCCCCAGTGCCTGCCAGTGCAACCCCCATGGCTCT
TACGGCGGCACCTGTGACCCAGCCACAGGCCAGTGCTCCTGCCGCCCAGGTGTGGGGGGCCTCAGGTG
TGACCGCTGTGAGCCTGGCTTCTGGAACTTTCGAGGCATCGTCACCGATGGCCGGAGTGGCTGTACAC
CCTGCAGCTGTGATCCCCAAGGCGCCGTGCGGGATGACTGTGAGCAGATGACGGGGCTGTGCTCGTGT
AAGCCCGGGGTGGCTGGACCCAAGTGTGGGCAGTGTCCAGACGGCCGTGCCCTGGGCCCCGCGGGCTG
TGAAGCTGACGCTTCTGCGCCTGCGACCTGTGCGGAGATGCGCTGTGAGTTCGGTGCGCGGTGCGTGG
AGGAGTCTGGCTCAGCCCACTGTGTCTGCCCGATGCTCACCTGTCCAGAGGCCAACGCTACCAAGGTC
TGTGGGTCAGATGGAGTCACATACGGCAACGAGTGTCAGCTGAAGACCATCGCCTGCCGCCAGGGCCT
GCAAATCTCTATCCAGAGCCTGGGCCCGTGCCAGGAGGCTGTTGCTCCCAGCACTCACCCGACATCTG
CCTCCGTGACTGTGACCACCCCAGGGCTCCTCCTGAGCCAGGCACTGCCGGCCCCCCCCGGCGCCCTC
CCCCTGGCTCCCAGCAGTACCGCACACAGCCAGACCACCCCTCCGCCCTCATCGCGACCTCGGACCAC
TGCCAGCGTCCCCAGGACCACCGTGTGGCCCGTGCTGACGGTGCCCCCCACGGCACCCTCCCCTGCAC
CCAGCCTGGTGGCGTCCGCCTTTGGTGAATCTGGCAGCACTGATGGAAGCAGCGATGAGGAACTGAGC
GGGGACCAGGAGGCCAGTGGGGGTGGCTCTGGGGGGCTCGAGCCCTTGGAGGGCAGCAGCGTGGCCAC
CCCTGGGCCACCTGTCGAGAGGGCTTCCTGCTACAACTCCGCGTTGGGCTGCTGCTCTGATGGGAAGA
CGCCCTCGCTGGACGCAGAGGGCTCCAACTGCCCCGCCACCAAGGTGTTCCAGGGCGTCCTGGAGCTG
GAGGGCGTCGAGGGCCAGGAGCTGTTCTACACGCCCGAGATGGCTGACCCCAAGTCAGAACTGTTCGG
GGAGACAGCCAGGAGCATTGAGAGCACCCTGGACGACCTCTTCCGGAATTCAGACGTCAAGAAGGATT
TCCGGAGTGTCCGCTTGCGGGACCTGGGGCCCGGCAAATCCGTCCGCGCCATTGTGGATGTGCACTTT
GACCCCACCACAGCCTTCAGGGCACCCGACGTGGCCCGGGCCCTGCTCCGGCAGATCCAGGTGTCCAG
GCGCCGGTCCTTGGGGGTGAGGCGGCCGCTGCAGGAGCACGTGCGATTTATGGACTTTGACTGGTTTC
CTGCGTTTATCACGGGGGCCACGTCAGGAGCCATTGCTGCGGGAGCCACGGCCAGAGCCACCACTGCA
TCGCGCCTGCCGTCCTCTGCTGTGACCCCTCGGGCCCCGCACCCCAGTCACACAAGCCAGCCCGTTGC
CAAGACCACGGCAGCCCCCACCACACGTCGGCCCCCCACCACTGCCCCCAGCCGTGTGCCCGGACGTC
GGCCCCCGGCCCCCCAGCAGCCTCCAAAGCCCTGTGACTCACAGCCCTGCTTCCACGGGGGGACCTGC
CAGGACTGGGCATTGGGCGGGGGCTTCACCTGCAGCTGCCCGGCAGGCAGGGGAGGCGCCGTCTGTGA
GAAGGTGCTTGGCGCCCCTGTGCCGGCCTTCGAGGGCCGCTCCTTCCTGGCCTTCCCCACCCTCCGCG
CCTACCACACGCTGCGCCTGGCACTGGAATTCCGGGCGCTGGAGCCTCAGGGGCTGCTGCTGTACAAT
GGCAACGCCCGGGGCAAGGACTTCCTGGCATTGGCGCTGCTAGATGGCCGCGTGCAGCTCAGGTTTGA
CACAGGTTCGGGGCCGGCGGTGCTGACCAGTGCCGTGCCGGTAGAGCCGGGCCAGTGGCACCGCCTGG
AGCTGTCCCGGCACTGGCGCCGGGGCACCCTCTCGGTGGATGGTGAGACCCCTGTTCTGGGCGAGAGT
CCCAGTGGCACCGACGGCCTCAACCTGGACACAGACCTCTTTGTGGGCGGCGTACCCGAGGACCAGGC
TGCCGTGGCGCTGGAGCGGACCTTCGTGGGCGCCGGCCTGAGGGGGTGCATCCGTTTGCTGGACGTCA
ACAACCAGCGCCTGGAGCTTGGCATTGGGCCGGGGGCTGCCACCCGAGGCTCTGGCGTGGGCGAGTGC
GGGGACCACCCCTGCCTGCCCAACCCCTGCCATGGCGGGGCCCCATGCCAGAACCTGGAGGCTGGAAG
GTTCCATTGCCAGTGCCCGCCCGGCCGCGTCGGACCAACCTGTGCCGATGAGAAGAGCCCCTGCCAGC
CCAACCCCTGCCATGGGGCGGCGCCCTGCCGTGTGCTGCCCGAGGGTGGTGCTCAGTGCGAGTGCCCC
CTGGGGCGTGAGGGCACCTTCTGCCAGACAGCCTCGGGGCAGGACGGCTCTGGGCCCTTCCTGGCTGA
CTTCAACGGCTTCTCCCACCTGGAGCTGAGAGGCCTGCACACCATTGCACGGGACCTGGGGGAGAAGA
TGGCGCTGGAGGCCGTGTTCCTGGCACGAGGCCCCAGCGGCCTCCTGCTCTACAACGGGCAGAAGACG
GACGGCAAGGGGGACTTCGTGTCGCTGGCACTGCGGGACCGCCGCCTGGAGTTCCGCTACGACCTGGG
CAAGGGGGCAGCGGTCATCAGGAGCAGGGAGCCAGTCACCCTGGGAGCCTGGACCAGGGTCTCACTGG
AGCGAAACGGCCGCAAGGGTGCCCTGCGTGTGGGCGACGGCCCCCGTGTGTTGGGGGAGTCCCCGAAA
TCCCGCAAGGTTCCGCACACCGTCCTCAACCTGAAGGAGCCGCTCTACGTAGGGGGCGCTCCCGACTT
CAGCAAGCTGGCCCGTGCTGCTGCCGTGTCCTCTGGCTTCGACGGTGCCATCCAGCTGGTCTCCCTCG
GAGGCCGCCAGCTGCTGACCCCGGAGCACGTGCTGCGGCAGGTGGACGTCACGTCCTTTGCAGGTCAC
CCCTGCACCCGGGCCTCAGGCCACCCCTGCCTCAATGGGGCCTCCTGCGTCCCGAGGGAGGCTGCCTA
TGTGTGCCTGTGTCCCGGGGGATTCTCAGGACCGCACTGCGAGAAGGGGCTGGTGGAGAAGTCAGCGG
GGGACGTGGATACCTTGGCCTTTGACGGGCGGACCTTTGTCGAGTACCTCAACGCTGTGACCGAGAGC
GAGAAGGCACTGCAGAGCAACCACTTTGAACTGAGCCTGCGCACTGAGGCCACGCAGGGGCTGGTGCT
CTGGAGTGGCAAGGCCACGGAGCGGGCAGACTATGTGGCACTGGCCATTGTGGACGGGCACCTGCAAC
TGAGCTACAACCTGGGCTCCCAGCCCGTGGTGCTGCGTTCCACCGTGCCCGTCAACACCAACCGCTGG
TTGCGGGTCGTGGCACATAGGGAGCAGAGGGAAGGTTCCCTGCAGGTGGGCAATGAGGCCCCTGTGAC
CGGCTCCTCCCCGCTGGGCGCCACGCAGCTGGACACTGATGGAGCCCTGTGGCTTGGGGGCCTGCCGG
AGCTGCCCGTGGGCCCAGCACTGCCCAAGGCCTACGGCACAGGCTTTGTGGGCTGCTTGCGGGATGTG
GTGGTGGGCCGGCACCCGCTGCACCTGCTGGAGGACGCCGTCACCAAGCCAGAGCTGCGGCCCTGCCC
CACCCCATGAGCTGGCACCAGAGCCCCGCGCCCGCT

| | | | |
|---|---|---|---|
| NOV60a, CG94946-01 | SEQ ID NO: 1174 | 2053 aa | MW at 215628.0kD |
| Protein Sequence | | | |

MRHGRPVPPGPAAGRPLLPLLVVAACVLPGAGGTCPERALERREEEANVVLTGTVEEILNVDPVQHTY
SCKVRVWRYLKGKDLVARESLLDGGNKVVISGFGDPLICDNQVSTGDTRTFFVNPAPPYLWPAHKNEL
MLNSSLMRITLRNLEEVEFCVEDKPGTHFTPVPPTPPDACRGMLCGFGAVCEPNAEGPGRASCVCKKS
PCPSVVAPVCGSDASTYSNECELQPAQCSQQRRIRLLSRGPCGSRDPCSNVTCSFGSTCARSADGLTA
SCLCPATCRGAPEGTVCGSDGADYPGECQLLRRACARQENVFKKFDGPCDPCQGALPDPSRSCRVNPR
TRRPEMLLRPESCPARQAPVCGDDGVTYENDCVMGRSGAARGLLLQKVRSGQCQGRDQCPEPCRFNAV
CLSRRGRPRCSCDRVTCDGAYRPVCAQDGRTYDSDCWRQQAECRQQRAIPSKHQGPCDQAPSPCLGVQ
CAFGATCAVKNGQAACECLQACSSLYDPVCGSDGVTYGSACELEATACTLGREIQVARKGPCDRCGQC
RFGALCEAETGRCVCPSECVALAQPVCGSDGHTYPSECMLHVHACTHQISLHVASAGPCETCGDAVCA
FGAVCSAGQCVCPRCEHPPPGPVCGSDGVTYGSACELREAACLQQTQIEEAPAGPCEQAECGSGGSGS
GEDGDCEQELCRQRGGIWDEDSEDGPCVCDFSCQSVPGSPVCGSDGVTYSTECELKKARCESQRGLYV
AAQGACRGPAFAPLPPVAPLHCAQTPYGCCQDNITAARGVGLAGCPSACQCNPHGSYGGTCDPATGQC
SCRPGVGGLRCDRCEPGFWNFRGIVTDGRSGCTPCSCDPQGAVRDDCEQMTGLCSCKPGVAGPKCGQC
PDGRALGPAGCEADASAPATCAEMRCEFGAPCVEESGSAHCVCPMLTCPEANATKVCGSDGVTYGNEC
QLKTIACRQGLQISIQSLGPCQEAVAPSTHPTSASVTVTTPGLLLSQALPAPPGALPLAPSSTAHSQT
TPPPSSRPRTTASVPRTTVWPVLTVPPTAPSPAPSLVASAFGESGSTDGSSDEELSGDQEASGGGSGG
LEPLEGSSVATPGPPVERASCYNSALGCCSDGKTPSLDAEGSNCPATKVFQGVLELEGVEGQELFYTP
EMADPKSELFGETARSTESTLDDLFRNSDVKKDFRSVRLRDLGPGKSVRAIVDVHFDPTTAFRAPDVA
RALLRQIQVSRRRSLGVRRPLQEHVRFMDFDWFPAFITGATSGAIAAGATARATTASRLPSSAVTPRA
PHPSHTSQPVAKTTAAPTTRRPPTTAPSRVPGRRPPAPQQPPKPCDSQPCFHGGTCQDWALGGGFTCS
CPAGRGGAVCEKVLGAPVPAFEGRSFLAFPTLRAYHTLRLALEFRALEPQGLLLYNGNARGKDFLALA
LLDGRVQLRFDTGSGPAVLTSAVPVEPGQWHRLELSRHWRRGTLSVDGETPVLGESPSGTDGLNLDTD
LFVGGVPEDQAAVALERTFVGAGLRGCIRLLDVNNQRLELGIGPGAATRGSGVGECGDHPCLPNPCHG
GAPCQNLEAGRFHCQCPPGRVGPTCADEKSPCQPNPCHGAAPCRVLPEGGAQCECPLGREGTFCQTAS
GQDGSGPFLADFNGFSHLELRGLHTIARDLGEKMALEAVFLARGPSGLLLYNGQKTDGKGDFVSLALR
DRRLEFRYDLGKGAAVIRSREPVTLGAWTRVSLERNGRKGALRVGDGPRVLGESPKSRKVPHTVLNLK
EPLYVGGAPDFSKLARAAAVSSGFDGAIQLVSLGGRQLLTPEHVLRQVDVTSFAGHPCTRASGHPCLN
GASCVPREAAYVCLCPGGFSGPHCEKGLVEKSAGDVDTLAFDGRTFVEYLNAVTESEKALQSNHFELS
LRTEATQGLVLWSGKATERADYVALAIVDGHLQLSYNLGSQPVVLRSTVPVNTNRWLRVVAHREQREG
SLQVGNEAPVTGSSPLGATQLDTDGALWLGGLPELPVGPALPKAYGTGFVGCLRDVVVGRHPLHLLED
AVTKPELRPCPTP

| | | |
|---|---|---|
| NOV60b, 275631590 | SEQ ID NO: 1175 | 388 bp |
| DNA Sequence | ORF Start: at 2 | ORF Stop: end of sequence |

CACCGGATCCGCCACCAAGGTGTTCCAGGGCGTCCTGGAGCTGGAGGGCGTCGAGGGCCAGGAGCTGT
TCTACACGCCCGAGATGGCTGACCCCAAGTCAGAACTGTTCGGGGAGACAGCCAGGAGCATTGAGAGC
ACCCTGGACGACCTCTTCCGGAATTCAGACGTCAAGAAGGATTTCCGGAGTGTCCGCTTGCGGGACCT
GGGGCCCGGCAAATCCGTCCGCGCCATTGTGGATGTGCACTTTGACCCCACCACAGCCTTCAGGGCAC
CCGACGTGGCCCGGGCCCTGCTCCGGCAGATCCAGGTGTCCAGGCGCCGGTCCTTGGGGGTGAGGCGG
CCGCTGCAGGAGCACGTGCGATTTATGGACTTTGACTGGGTCGACGGC

| | | | |
|---|---|---|---|
| NOV60b, 275631590 | SEQ ID NO: 1176 | 129 aa | MW at 14652.4kD |
| Protein Sequence | | | |

TGSATKVFQGVLELEGVEGQELFYTPEMADPKSELFGETARSIESTLDDLFRNSDVKKDFRSVRLRDL
GPGKSVRAIVDVHFDPTTAFRAPDVARALLRQIQVSRRRSLGVRRPLQEHVRFMDFDWVDG

| | | |
|---|---|---|
| NOV60c, 275631564 | SEQ ID NO: 1177 | 316 bp |
| DNA Sequence | ORF Start: at 2 | ORF Stop: end of sequence |

CACCGGATCCTGCCAGTGCAACCCCCATGGCTCTTACGGCGGCACCTGTGACCCAGCCACAGGCCAGT
GCTCCTGCCGCCCAGGTGTGGGGGGCCTCAGGTGTGACCGCTGTGAGCCTGGCTTCTGGAACTTTCGA
GGCATCGTCACCGATGGCCGGAGTGGCTGTACACCCTGCAGCTGTGATCCCCAAGGCGCCGTGCGGGA
TGACTGTGAGCAGATGACGGGGCTGTGCTCGTGTAAGCCCGGGGTGGCTGGACCCAAGTGTGGGCAGT
GTCCAGACGGCCGTGCCCTGGGCCCCGCGGGCTGTGTCGACGGC

| | | | |
|---|---|---|---|
| NOV60c, 275631564 | SEQ ID NO: 1178 | 105 aa | MW at 10579.7kD |
| Protein Sequence | | | |

TGSCQCNPHGSYGGTCDPATGQCSCRPGVGGLRCDRCEPGFWNFRGIVTDGRSGCTPCSCDPQGAVRD
DCEQMTGLCSCKPGVAGPKCGQCPDGRALGPAGCVDG

| | | |
|---|---|---|
| NOV60d, CG94946-02 | SEQ ID NO: 1179 | 4760 bp |
| DNA Sequence | ORF Start: ATG at 37 | ORF Stop: TGA at 4732 |

CCGGCGCGGCCCGCGCGCTCTTCCGCCGCCTCTCGCATGCGCCATGGCCGGCCGGTCCCACCCGGGCC
CGCTGCGGGGCGGCCGCTGCTGCCTCTCCTTGTGGTGGCCGCGTGCGTCCTGCCCGGAGCCGGCGGGA
CATGCCCGGAGCGCGCGCTGGAGCGGCGCGAGGAGGAGGCGAACGTGGTGCTCACCGGGACGGTGGAG
GAGATCCTCAACGTGGACCCGGTGCAGCACACGTACTCCTGCAAGGTTCGGGTCTGGCGGTACTTGAA
GGGCAAAGACCTGGTGGCCCGGGAGAGCCTGCTGGACGGCGGCAACAAGGTGGTGATCAGCGGCTTTG
GAGACCCCCTCATCTGTGACAACCAGGTGTCCACTGGGGACACCAGGATCTTCTTTGTGAACCCTGCA
CCCCCATACCTGTGGCCAGCCCACAAGAACGAGCTGATGCTCAACTCCAGCCTCATGCGGATCACCCT
GCGGAACCTGGAGGAGGTGGAGTTCTGTGTGGAAGATAAACCCGGGACCCACTTCACTCCAGTGCCTC
CGACGCCTCCTGATGCGTGCCGGGGAATGCTGTGCGGCTTCGGCGCCGTGTGCGAGCCCAACGCGGAG
GGGCCGGGCCGGGCGTCCTGCGTCTGCAAGAAGAGCCCGTGCCCCAGCGTGGTGGCGCCTGTGTGTGG
GTCGGACGCCTCCACCTACAGCAACGAATGCGAGCTGCAGCGGGCGCAGTGCAGCCAGCAGCGCCGCA
TCCGCCTGCTCAGCCGCGGGCCGTGCGGCTCGCGGGACCCCTGCTCCAACGTGACCTGCAGCTTCGGC
AGCACCTGTGCGCGCTCGGCCGACGGGCTGACGGCCTCGTGCCTGTGCCCCGCGACCTGCCGTGGCGC
CCCCGAGGGGACCGTCTGCGGCAGCGACGGCGCCGACTACCCCGGCGAGTGCCAGCTCCTGCGCCGCG
CCTGCGCCCGCCAGGAGAATGTCTTCAAGAAGTTCGACGGCCCTTGTGACCCCTGTCAGGGCGCCCTC
CCTGACCCGAGCCGCAGCTGCCGTGTGAACCCGCGCACGCGGCGCCCTGAGATGCTCCTACGGCCCGA
GAGCTGCCCTGCCCGGCAGGCGCCAGTGTGTGGGGACGACGGAGTCACCTACGAAAACGACTGTGTCA
TGGGCCGATCGGGGGCCGCCCGGGGTCTCCTCCTGCAGAAAGTGCGCTCCGGCCAGTGCCAGGGTCGA
GACCAGTGCCCGGAGCCCTGCCGGTTCAATGCCGTGTGCCTGTCCCGCCGTGGCCGTCCCCGCTGCTC
CTGCGACCGCGTCACCTGTGACGGGGCCTACAGGCCCGTGTGTGCCCAGGACGGGCGCACGTATGACA
GTGATTGCTGGCGGCAGCAGGCTGAGTGCCGGCAGCAGCGTGCCATCCCCAGCAAGCACCAGGGCCCG
TGTGACCAGGCCCCGTCCCCATGCCTCGGGGTGCAGTGTGCATTTGGGGCGACGTGTGCTGTGAAGAA
CGGGCAGGCAGCGTGTGAATGCCTGCAGGCGTGCTCGAGCCTCTACGATCCTGTGTGCGGCAGCGACG
GCGTCACATACGGCAGCGCGTGCGAGCTGGAGGCCACGGCCTGTACCCTCGGGCGGGAGATCCAGGTG
GCGCGCAAAGGACCCTGTGACCGCTGCGGGCAGTGCCGCTTTGGAGCCCTGTGCGAGGCCGAGACCGG
GCGCTGCGTGTGCCCCTCTGAATGCGTGGCTTTGGCCCAGCCCGTGTGTGGCTCCGACGGGCACACGT
ACCCCAGCGAGTGCATGCTGCACGTGCACGCCTGCACACACCAGATCAGCCTGCACGTGGCCTCAGCT
GGACCCTGTGAGACCTGTGGAGATGCCGTGTGTGCTTTTGGGGCTGTGTGCTCCGCAGGGCAGTGTGT
GTGTCCCCGGTGTGAGCACCCCCCGCCCGGCCCCGTGTGTGGCAGCGACGGTGTCACCTACGGCAGTG
CCTGCGAGCTACGGGAAGCCGCCTGCCTCCAGCAGACACAGATCGAGGAGGCCCGGGCAGGGCCGTGC
GAGCAGGCCGAGTGCGGTTCCGGAGGCTCTGGCTCTGGGGAGGACGGTGACTGTGAGCAGGAGCTGTG
CCGGCAGCGCGGTGGCATCTGGGACGAGGACTCGGAGGACGGGCCGTGTGTCTGTGACTTCAGCTGCC
AGAGTGTCCCAGGCAGCCCGGTGTGCGGCTCAGATGGGGTCACCTACAGCACCGAGTGTGAGCTGAAG
AAGGCCAGGTGTGAGTCACAGCGAGGGCTCTACGTAGCGGCCCAGGGAGCCTGCCGAGGCCCCGCCTT
CGCCCCGCTGCCGCCTGTGGCCCCCTTACACTGTGCCCAGACGCCCTACGGCTGCTGCCAGGACAATA
TCACCGCAGCCCGGGGCGTGGGCCTGGCTGGCTGCCCCAGTGCCTGCCAGTGCAACCCCCATGGCTCT
TACGGCGGCACCTGTGACCCAGCCACAGGCCAGTGCTCCTGCCGCCCAGGTGTGGGGGGCCTCAGGTG
TGACCGCTGTGAGCCTGGCTTCTGGAACTTTCGAGGCATCGTCACCGATGGCCGGAGTGGCTGTACAC
CCTGCAGCTGTGATCCCCAAGGCGCCGTGCGGGATGACTGTGAGCAGATGACGGGGCTGTGCTCGTGT
AAGCCCGGGGTGGCTGGACCCAAGTGTGGGCAGTGTCCAGACGGCCGTGCCCTGGGCCCCGCGGGCTG
TGAAGCTGACGCTTCTGCGCCTGCGACCTGTGCGGAGATGCGCTGTGAGTTCGGTGCGCGGTGCGTGG
AGGAGTCTGGCTCAGCCCACTGTGTCTGCCCGATGCTCACCTGTCCAGAGGCCAACGCTACCAAGGTC
TGTGGGTCAGATGGAGTCACATACGGCAACGAGTGTCAGCTGAAGACCATCGCCTGCCGCCAGGGCCT
GCAAATCTCTATCCAGAGCCTGGGCCCGTGCCAGGAGGCTGTTGCTCCCAGCACTCACCCGACATCTG
CCTCCGTGACTGTGACCACCCCAGGGCTCCTCCTGAGCCAGGCACTGCCGGCCCCCCCCGGCGCCCTC
CCCCTGGCTCCCAGCAGTACCGCACACAGCCAGACCACCCCTCCGCCCTCATCGCGACCTCGGACCAC
TGCCAGCGTCCCCAGGACCACCGTGTGGCCCGTGCTGACGGTGCCCCCCACGGCACCCTCCCCTGCAC
CCAGCCTGGTGGCGTCCGCCTTTGGTGAATCTGGCAGCACTGATGGAAGCAGCGATGAGGAACTGAGC
GGGGACCAGGAGGCCAGTGGGGGTGGCTCTGGGGGGCTCGAGCCCTTGGAGGGCAGCAGCGTGGCCAC
CCCTGGGCCACCTGTCGAGAGGGCTTCCTGCTACAACCCCTGCCATGGGGCGGCGCCCTGCCGTGTGC
TGCCCGAGGGTGGTGCTCAGTGCGAGTGCCCCCTGGGGCGTGAGGGCACCTTCTGCCAGACAGCCTCG
GGGCAGGACGGCTCTGGGCCCTTCCTGGCTGACTTCAACGGCTTCTCCCACCTGGAGCTGAGAGGCCT
GCACACCATTGCACGGGACCTGGGGGAGAAGATGGCGCTGGAGGCCGTGTTCCTGGCACGAGGCCCCA
GCGGCCTCCTGCTCTACAACGGGCAGAAGACGGACGGCAAGGGGGACTTCGTGTCGCTGGCACTGCGG
GACCGCCGCCTGGAGTTCCGCTACGACCTGGGCAAGGGGGCAGCGGTCATCAGGAGCAGGGAGCCAGT
CACCCTGGGAGCCTGGACCAGGGTCTCACTGGAGCGAAACGGCCGCAAGGGTGCCCTGCGTGTGGGCG
ACGGCCCCCGTGTGTTGGGGGAGTCCCCGAAATCCCGCAAGGTTCCGCACACCGTCCTCAACCTGAAG
GAGCCGCTCTACGTAGGGGGCGCTCCCGACTTCAGCAAGCTGGCCCGTGCTGCTGCCGTGTCCTCTGG
CTTCGACGGTGCCATCCAGCTGGTCTCCCTCGGAGGCCGCCAGCTGCTGACCCCGGAGCACGTGCTGC
GGCAGGTGGACGTCACGTCCTTTGCAGGTCACCCCTGCACCCGGGCCTCAGGCCACCCCTGCCTCAAT
GGGGCCTCCTGCGTCCCGAGGGAGGCTGCCTATGTGTGCCTGTGTCCCGGGGGATTCTCAGGACCGCA
CTGCGAGAAGGGGCTGGTGGAGAAGTCAGCGGGGGACGTGGATACCTTGGCCTTTGACGGGCGGACCT
TTGTCGAGTACCTCAACGCTGTGACCGAGAGCGAGAAGGCACTGCAGAGCAACCACTTTGAACTGAGC
CTGCGCACTGAGGCCACGCAGGGGCTGGTGCTCTGGAGTGGCAAGGCCACGGAGCGGGCAGACTATGT
GGCACTGGCCATTGTGGACGGGCACCTGCAACTGAGCTACAACCTGGGCTCCCAGCCCGTGGTGCTGC
GTTCCACCGTGCCCGTCAACACCAACCGCTGGTTGCGGGTCGTGGCACATAGGGAGCAGAGGGAAGGT
TCCCTGCAGGTGGGCAATGAGGCCCCTGTGACCGGCTCCTCCCCGCTGGGCGCCACGCAGCTGGACAC
TGATGGAGCCCTGTGGCTTGGGGGCCTGCCGGAGCTGCCCGTGGGCCCAGCACTGCCCAAGGCCTACG
GCACAGGCTTTGTGGGCTGCTTGCGGGATGTGGTGGTGGGCCGGCACCCGCTGCACCTGCTGGAGGAC
GCCGTCACCAAGCCAGAGCTGCGGCCCTGCCCCACCCCATGAGCTGGCACCAGAGCCCCGCGCCCGCT

| | | | |
|---|---|---|---|
| NOV60d, CG94946-02 | SEQ ID NO: 1180 | 1565 aa | MW at 163817.1kD |
| Protein Sequence | | | |

MRHGRPVPPGPAAGRPLLPLLVVAACVLPGAGGTCPERALERREEEANVVLTGTVEEILNVDPVQHTY
SCKVRVWRYLKGKDLVARESLLDGGNKVVISGFGDPLICDNQVSTGDTRIFFVNPAPPYLWPAHKNEL
MLNSSLMRITLRNLEEVEFCVEDKPGTHFTPVPPTPPDACRGMLCGFGAVCEPNAEGPGRASCVCKKS
PCPSVVAPVCGSDASTYSNECELQRAQCSQQPRIRLLSRGPCGSRDPCSNVTCSFGSTCARSADGLTA
SCLCPATCRGAPEGTVCGSDGADYPGECQLLRRACARQENVFKKFDGPCDPCQGALPDPSRSCRVNPR
TRRPEMLLRPESCPARQAPVCGDDGVTYENDCVMGRSGAARGLLLQKVRSGQCQGRDQCPEPCRFNAV
CLSRRGRPRCSCDRVTCDGAYRPVCAQDGRTYDSDCWRQQAECRQQRAIPSKHQGPCDQAPSPCLGVQ
CAFGATCAVKNGQAACECLQACSSLYDPVCGSDGVTYGSACELEATACTLGREIQVARKGPCDRCGQC
RFGALCEAETGRCVCPSECVALAQPVCGSDGHTYPSECMLHVHACTHQISLHVASAGPCETCGDAVCA
FGAVCSAGQCVCPRCEHPPPGPVCGSDGVTYGSACELREAACLQQTQIEEARAGPCEQAECGSGGSGS
GEDGDCEQELCRQRGGIWDEDSEDGPCVCDFSCQSVPGSPVCGSDGVTYSTECELKKARCESQRGLYV
AAQGACRGPAFAPLPPVAPLHCAQTPYGCCQDNITAARGVGLAGCPSACQCNPHGSYGGTCDPATGQC
SCRPGVGGLRCDRCEPGFWNFRGIVTDGRSGCTPCSCDPQGAVRDDCEQMTGLCSCKPGVAGPKCGQC
PDGRALGPAGCEADASAPATCAEMRCEFGARCVEESGSAHCVCPMLTCPEANATKVCGSDGVTYGNEC
QLKTIACRQGLQISIQSLGPCQEAVAPSTHPTSASVTVTTPGLLLSQALPAPPGALPLAPSSTAHSQT
TPPPSSRPRTTASVPRTTVWPVLTVPPTAPSPAPSLVASAFGESGSTDGSSDEELSGDQEASGGGSGG
LEPLEGSSVATPGPPVERASCYNPCHGAAPCRVLPEGGAQCECPLGREGTFCQTASGQDGSGPFLADF
NGFSHLELRGLHTIARDLGEKMALEAVFLARGPSGLLLYNGQKTDGKGDFVSLALRDRRLEFRYDLGK
GAAVIRSREPVTLGAWTRVSLERNGRKGALRVGDGPRVLGESPKSRKVPHTTLNLKEPLYVGGAPDFS
KLARAAAVSSGFDGAIQLVSLGGRQLLTPEHVLRQVDVTSFAGHPCTRASGHPCLNGASCVPREAAYV
CLCPGGFSGPHCEKGLVEKSAGDVDTLAFDGRTFVEYLNAVTESEKALQSNHFELSLRTEATQGLVLW
SGKATERADYVALATVDGHLQLSYNLGSQPVVLRSTVPVNTNRWLRVVAHREQREGSLQVGNEAPVTG
SSPLGATQLDTDGALWLGGLPELPVGPALPKAYGTGFVGCLRDVVVGRHPLHLLEDAVTKPELRPCPT
P

| | | |
|---|---|---|
| NOV60e, CG94946-03 | SEQ ID NO: 1181 | 893 bp |
| DNA Sequence | ORF Start: ATG at 37 | ORF Stop: TGA at 865 |

CCGGCGCGGCCCGCGCGCTCTTCCGCCGCCTCTCGCATGCGCCATGGCCGGCCGGTCCCACCCGGGCC
CGCTGCGGGGCGGCCGCTGCTGCCTCTCCTTGTGGTGGCCGCGTGCGTCCTGCCCGGAGCCGGCGGGA
CATGCCCGGAGCGCGCGCTGGAGCGGCGCGAGGAGGAGGCGAACGTGGTGCTCACCGGGACGGTGGAG
GAGATCCTCAACGTGGACCCGGTGCAGCACACGTACTCCTGCAAGGTTCGGGTCTGGCGGTACTTGAA
GGGCAAAGACCTGGTGGCCCGGGAGAGCCTGCTGGACGGCGGCAACAAGGTGGTGATCAGCGGCTTTG
GAGACCCCCTCATCTGTGACAACCAGGTGTCCACTGGGGACACCAGGATCTTCTTTGTGAACCCTGCA
CCCCCATACCTGTGGCCAGCCCACAAGAACGAGCTGGTGCTCTGGAGTGGCAAGGCCACGGAGCGGGC
AGACTATGTGGCACTGGCCATTGTGGACGGGCACCTGCAACTGAGCTACAACCTGGGCTCCCAGCCCG
TGGTGCTGCGTTCCACCGTGCCCGTCAACACCAACCGCTGGTTGCGGGTCGTGGCACATAGGGAGCAG
AGGGAAGGTTCCCTGCAGGTGGGCAATGAGGCCCCTGTGACCGGCTCCTCCCCGCTGGGCGCCACGCA
GCTGGACACTGATGGAGCCCTGTGGCTTGGGGGCCTGCCGGAGCTGCCCGTGGGCCCAGCACTGCCCA
AGGCCTACGGCACAGGCTTTGTGGGCTGCTTGCGGGATGTGGTGGTGGGCCGGCACCCGCTGCACCTG
CTGGAGGACGCCGTCACCAAGCCAGAGCTGCGGCCCTGCCCCACCCCATGAGCTGGCACCAGAGCCCC
GCGCCCGCT

| | | | |
|---|---|---|---|
| NOV60e, CG94946-03 | SEQ ID NO: 1182 | 276 aa | MW at 29688.8kD |
| Protein Sequence | | | |

MRHGRPVPPGPAAGRPLLPLLVVAACVLPGAGGTCPERALERREEEANVVLTGTVEEILNVDPVQHTY
SCKVRVWRYLKGKDLVARESLLDGGNKVVISGFGDPLICDNQVSTGDTRIFFVNPAPPYLWPAHKNEL
VLWSGKATERADYVALAIVDGHLQLSYNLGSQPVVLRSTVPVNTNRWLRVVAHREQREGSLQVGNEAP
VTGSSPLGATQLDTDGALWLGGLPELPVGPALPKAYGTGFVGCLRDVVVGRHPLHLLEDAVTKPELRP
CPTP

| | | |
|---|---|---|
| NOV60f, CG94946-04 | SEQ ID NO: 1183 | 1931 bp |
| DNA Sequence | ORF Start: ATG at 37 | ORF Stop: TGA at 1903 |

CCGGCGCGGCCCGCGCGCTCTTCCGCCGCCTCTCGCATGCGCCATGGCCGGCCGGTCCCACCCGGGCC
CGCTGCGGGGCGGCCGCTGCTGCCTCTCCTTGTGGTGGCCGCGTGCGTCCTGCCCGGAGCCGGCGGGA
CATGCCCGGAGCGCGCGCTGGAGCGGCGCGAGGAGGAGGCGAACGTGGTGCTCACCGGGACGGTGGAG
GAGATCCTCAACGTGGACCCGGTGCAGCACACGTACTCCTGCAAGGTTCGGGTCTGGCGGTACTTGAA
GGGCAAAGACCTGGTGGCCCGGGAGAGCCTGCTGGACGGCGGCAACAAGGTGGTGATCAGCGGCTTTG
GAGACCCCCTCATCTGTGACAACCAGGTGTCCACTGGGGACACCAGGATCTTCTTTGTGAACCCTGCA
CCCCCATACCTGTGGCCAGCCCACAAGAACGAGCTGATGCTCAACTCCAGCCTCATGCGGATCACCCT
GCGGAACCTGGAGGAGGTGGAGTTCTGTGTGGAAGATAAACCCGGGACCCACTTCACTCCAGTGCCTC
CGACGCCTCCTGATGCGTGCCGGGGAATGCTGTGCGGCTTCGGCGCCGTGTGCGAGCCCAACGCGGAG
GGGCCGGGCCGGGCGTCCTGCGTCTGCAAGAAGAGCCCGTGCCCCAGCGTGGTGGCGCCTGTGTGTGG
GTCGGACGCCTCCACCTACAGCAACGAATGCGAGCTGCAGCGGGCGCAGTGCAGCCAGCAGCGCCGCA
TCCGCCTGCTCAGCCGCGGGCCGTGCGGCTCGCGGGACCCCTGCTCCAACGTGACCTGCAGCTTCGGC
AGCACCTGTGCGCGCTCGGCCGACGGGCTGACGGCCTCGTGCCTGTGCCCCGCGACCTGCCGTGGCGC
CCCCGAGGGGACCGTCTGCGGCAGCGACGGCGCCGACTACCCCGGCGAGTGCCAGCTCCTGCGCCGCG
CCTGCGCCCGCCAGGAGAATGTCTTCAAGAAGTTCGACGGCCCTTGTGACCCCTGTCAGGGCGCCCTC
CCTGACCCGAGCCGCAGCTGCCGTGTGAACCCGCGCACGCGGCGCCCTGAGATGCTCCTACGGCCCGA
GAGCTGCCCTGCCCGGCAGGCGCCAGTGTGTGGGGACGACGGAGTCACCTACGAAAACGACTGTGTCA
TGGGCCGATCGGGGGCCGCCCGGGGTCTCCTCCTGCAGAAAGTGCGCTCCGGCCAGTGCCAGGGTCGA
GACCAGTGCCCGGAGCCCTGCCGGTTCAATGCCGTGTGCCTGTCCCGCCGTGGCCGTCCCCGCTGCTC
CTGCGACCGCGTCACCTGTGACGGGGCCTACAGGCCCGTGTGTGCCCAGGACGGGCGCACGTATGACA
GTGATTGCTGGCGGCAGCAGGCTGAGTGCCGGCAGCAGCGTGCCATCCCCAGCAAGCACCAGGGCCCG
TGTGACCAGGCCCCGTCCCCATGCCTCGGGGTGCAGTGTGCATTTGGGGCGACGTGTGCTGTGAAGAA
CGGGCAGGCAGCGTGTGAATGCCTGCAGGCGTGCTCGAGCCTCTACGATCCTGTGTGCGGCAGCGACG
GCGTCACATACGGCAGCGCGTGCGAGCTGGAGGCCACGGCCTGTACCCTCGGGCGGGAGATCCAGGTG
GCGCGCAAAGGACCCTGTGACCGCTGCGGGCAGTGCCGCTTTGGAGCCCTGCCTGTGACCGGCTCCTC
CCCGCTGGGCGCCACGCAGCTGGACACTGATGGAGCCCTGTGGCTTGGGGGCCTGCCGGAGCTGCCCG
TGGGCCCAGCACTGCCCAAGGCCTACGGCACAGGCTTTGTGGGCTGCTTGCGGGATGTGGTGGTGGGC
CGGCACCCGCTGCACCTGCTGGAGGACGCCGTCACCAAGCCAGAGCTGCGGCCCTGCCCCACCCCATG
AGCTGGCACCAGAGCCCCGCGCCCGCT

| | | | |
|---|---|---|---|
| NOV60f, CG94946-04 | SEQ ID NO: 1184 | 622 aa | MW at 66353.9kD |
| Protein Sequence | | | |
| MRHGRPVPPGPAAGRPLLPLLVVAACVLPGAGGTCPERALERREEEAIWVLTGTVEEILNVDPVQHTY | | | |
| SCKVRVWRYLKGKDLVARESLLDGGNKVVISGFGDPLICDNQVSTGDTRIFFVNPAPPYLWPAHKNEL | | | |
| MLNSSLMRITLRNLEEVEFCVEDKPGTHFTPVPPTPPDACRGMLCGFGAVCEPNAEGPGRASCVCKKS | | | |
| PCPSVVAPVCGSDASTYSNECELQRAQCSQQRRIRLLSRGPCGSRDPCSNVTCSFGSTCARSADGLTA | | | |
| SCLCPATCRGAPEGTVCGSDGADYPGECQLLRRACARQENVFKKFDGPCDPCQGALPDPSRSCRVNPR | | | |
| TRRPEMLLRPESCPARQAPVCGDDGVTYENDCVMGRSGAARGLLLQKVRSGQCQGRDQCPEPCRFNAV | | | |
| CLSRRGRPRCSCDRVTCDGAYRPVCAQDGRTYDSDCWRQQAECRQQRAIPSKHQGPCDQAPSPCLGVQ | | | |
| CAFGATCAVKNGQAACECLQACSSLYDPVCGSDGVTYGSACELEATACTLGREIQVARKGPCDRCGQC | | | |
| RFGALPVTGSSPLGATQLDTDGALWLGGLPELPVGPALPKAYGTGFVGCLRDVVVGRHPLHLLEDAVT | | | |
| KPELRPCPTP | | | |

| | | |
|---|---|---|
| NOV60g, CG94946-05 | SEQ ID NO: 1185 | 4697 bp |
| DNA Sequence | ORF Start: ATG at 37 | ORF Stop: TGA at 4669 |

CCGGCGCGGCCCGCGCGCTCTTCCGCCGCCTCTCGCATGCGCCATGGCCGGCCGGTCCCACCCGGGCC
CGCTGCGGGGCGGCCGCTGCTGCCTCTCCTTGTGGTGGCCGCGTGCGTCCTGCCCGGAGCCGGCGGGA
CATGCCCGGAGCGCGCGCTGGAGCGGCGCGAGGAGGAGGCGAACGTGGTGCTCACCGGGACGGTGGAG
GAGATCCTCAACGTGGACCCGGTGCAGCACACGTACTCCTGCAAGGTTCGGGTCTGGCGGTACTTGAA
GGGCAAAGACCTGGTGGCCCGGGAGAGCCTGCTGGACGGCGGCAACAAGGTGGTGATCAGCGGCTTTG
GAGACCCCCTCATCTGTGACAACCAGGTGTCCACTGGGGACACCAGGATCTTCTTTGTGAACCCTGCA
CCCCCATACCTGTGGCCAGCCCACAAGAACGAGCTGATGCTCAACTCCAGCCTCATGCGGATCACCCT
GCGGAACCTGGAGGAGGTGGAGTTCTGTGTGGAAGATAAACCCGGGACCCACTTCACTCCAGTGCCTC
CGACGCCTCCTGATGCGTGCCGGGGAATGCTGTGCGGCTTCGGCGCCGTGTGCGAGCCCAACGCGGAG
GGGCCGGGCCGGGCGTCCTGCGTCTGCAAGAAGAGCCCGTGCCCCAGCGTGGTGGCGCCTGTGTGTGG
GTCGGACGCCTCCACCTACAGCAACGAATGCGAGCTGCAGCGGGCGCAGTGCAGCCAGCAGCGCCGCA
TCCGCCTGCTCAGCCGCGGGCCGTGCGGCTCGCGGGACCCCTGCTCCAACGTGACCTGCAGCTTCGGC
AGCACCTGTGCGCGCTCGGCCGACGGGCTGACGGCCTCGTGCCTGTGCCCCGCGACCTGCCGTGGCGC
CCCCGAGGGGACCGTCTGCGGCAGCGACGGCGCCGACTACCCCGGCGAGTGCCAGCTCCTGCGCCGCG
CCTGCGCCCGCCAGGAGAATGTCTTCAAGAAGTTCGACGGCCCTTGTGACCCCTGTCAGGGCGCCCTC
CCTGACCCGAGCCGCAGCTGCCGTGTGAACCCGCGCACGCGGCGCCCTGAGATGCTCCTACGCCCCGA
GAGCTGCCCTGCCCGGCAGGCGCCAGTGTGTGGGGACGACGGAGTCACCTACGAAAACGACTGTGTCA
TGGGCCGATCGGGGGCCGCCCGGGGTCTCCTCCTGCAGAAAGTGCGCTCCGGCCAGTGCCAGGGTCGA
GACCAGTGCCCGGAGCCCTGCCGGTTCAATGCCGTGTGCCTGTCCCGCCGTGGCCGTCCCCGCTGCTC
CTGCGACCGCGTCACCTGTGACGGGGCCTACAGGCCCGTGTGTGCCCAGGACGGGCGCACGTATGACA
GTGATTGCTGGCGGCAGCAGGCTGAGTGCCGGCAGCAGCGTGCCATCCCCAGCAAGCACCAGGGCCCG
TGTGACCAGGCCCCGTCCCCATGCCTCGGGGTGCAGTGTGCATTTGGGGCGACGTGTGCTGTGAAGAA
CGGGCAGGCAGCGTGTGAATGCCTGCAGGCGTGCTCGAGCCTCTACGATCCTGTGTGCGGCAGCGACG
GCGTCACATACGGCAGCGCGTGCGAGCTGGAGGCCACGGCCTGTACCCTCGGGCGGGAGATCCAGGTG
GCGCGCAAAGGACCCTGTGACCGCTGCGGGCAGTGCCGCTTTGGAGCCCTGTGCGAGGCCGAGACCGG
GCGCTGCGTGTGCCCCTCTGAATGCGTGGCTTTGGCCCAGCCCGTGTGTGGCTCCGACGGGCACACGT
ACCCCAGCGAGTGCATGCTGCACGTGCACGCCTGCACACACCAGATCAGCCTGCACGTGGCCTCAGCT
GGACCCTGTGAGACCTGTGGAGATGCCGTGTGTGCTTTTGGGGCTGTGTGCTCCGCAGGGCAGTGTGT
GTGTCCCCGGTGTGAGCACCCCCCGCCCGGCCCCGTGTGTGGCAGCGACGGTGTCACCTACGGCAGTG
CCTGCGAGCTACGGGAAGCCGCCTGCCTCCAGCAGACACAGATCGAGGAGGCCCGGGCAGGGCCGTGC
GAGCAGGCCGAGTGCGGTTCCGGAGGCTCTGGCTCTGGGGAGGACGGTGACTGTGAGCAGGAGCTGTG
CCGGCAGCGCGGTGGCATCTGGGACGAGGACTCGGAGGACGGGCCGTGTGTCTGTGACTTCAGCTGCC
AGAGTGTCCCAGGCAGCCCGGTGTGCGGCTCAGATGGGGTCACCTACAGCACCGAGTGTGAGCTGAAG
AAGGCCAGGTGTGAGTCACAGCGAGGGCTCTACGTAGCGGCCCAGGGAGCCTGCCGAGGCCCCGCCTT
CGCCCCGCTGCCGCCTGTGGCCCCCTTACACTGTGCCCAGACGCCCTACGGCTGCTGCCAGGACAATA
TCACCGCAGCCCGGGGCGTGGGCCTGGCTGGCTGCCCCAGTGCCTGCCAGTGCAACCCCCATGGCTCT
TACGGCGGCACCTGTGACCCAGCCACAGGCCAGTGCTCCTGCCGCCCAGGTGTGGGGGGCCTCAGGTG
TGACCGCTGTGAGCCTGGCTTCTGGAACTTTCGAGGCATCGTCACCGATGGCCGGAGTGGCTGTACAC
CCTGCAGCTGTGATCCCCAAGGCGCCGTGCGGGATGACTGTGAGCAGATGACGGGGCTGTGCTCGTGT
AAGCCCGGGGTGGCTGGACCCAAGTGTGGGCAGTGTCCAGACGGCCGTGCCCTGGGCCCCGCGGGCTG
TGAAGCTGACGCTTCTGCGCCTGCGACCTGTGCGGAGATGCGCTGTGAGTTCGGTGCGCGGTGCGTGG
AGGAGTCTGGCTCAGCCCACTGTGTCTGCCCGATGCTCACCTGTCCAGAGGCCAACGCTACCAAGGTC
TGTGGGTCAGATGGAGTCACATACGGCAACGAGTGTCAGCTGAAGACCATCGCCTGCCGCCAGGGCCT
GCAAATCTCTATCCAGAGCCTGGGCCCGTGCCAGGAGGCTGTTGCTCCCAGCACTCACCCGACATCTG
CCTCCGTGACTGTGACCACCCCAGGGCTCCTCCTGAGCCAGGCACTGCCGGCCCCCCCCGGCGCCCTC
CCCCTGGCTCCCAGCAGTACCGCACACAGCCAGACCACCCCTCCGCCCTCATCGCGACCTCGGACCAC
TGCCAGCGTCCCCAGGACCACCGTGTGGCCCGTGCTGACGGTGCCCCCCACGGCACCCTCCCCTGCAC
CCAGCCTGGTGGCGTCCGCCTTTGGTGAATCTGGCAGCACTGATGGAAGCAGCGATGAGGAACTGAGC
GGGGACCAGGAGGCCAGTGGGGGTGGCTCTGGGGGGCTCGAGCCCTTGGAGGGCAGCAGCGTGGCCAC
CCCTGGGCCACCTGTCGAGAGGGCTTCCTGCTACAACTCCGCGTTGGGCTGCTGCTCTGATGGGAAGA
CGCCCTCGCTGGACGCAGAGGGCTCCAACTGCCCCGCCACCAAGGTGTTCCAGGGCGTCCTGGAGCTG
GAGGGCGTCGAGGGCCAGGAGCTGTTCTACACGCCCGAGATGGCTGACCCCAAGTCAGAACTGTTCGG
GGAGACAGCCAGGAGCATTGAGAGCACCCTGGACGACCTCTTCCGGAATTCAGACGTCAAGAAGGATT
TCCGGAGTGTCCGCTTGCGGGACCTGGGGCCCGGCAAATCCGTCCGCGCCATTGTGGATGTGCACTTT
GACCCCACCACAGCCTTCAGGGCACCCGACGTGGCCCGGGCCCTGCTCCGGCAGATCCAGGTGTCCAG
GCGCCGGTCCTTGGGGGTGAGGCGGCCGCTGCAGGAGCACGTGCGATTTATGGACTTTGACTGGTTTC
CTGCGTTTATCACGGGGGCCACGTCAGGAGCCATTGCTGCGGGAGCCACGGCCAGAGCCACCACTGCA
TCGCGCCTGCCGTCCTCTGCTGTGACCCCTCGGGCCCCGCACCCCAGTCACACAAGCCAGCCCGTTGC
CAAGACCACGGCAGCCCCCACCACACGTCGGCCCCCCACCACTGCCCCCAGCCGTGTGCCCGGACGTC
GGCCCCCGGCCTCCTGCGTCCCGAGGGAGGCTGCCTATGTGTGCCTGTGTCCCGGGGGATTCTCAGGA
CCGCACTGCGAGAAGGGGCTGGTGGAGAAGTCAGCGGGGGACGTGGATACCTTGGCCTTTGACGGGCG
GACCTTTGTCGAGTACCTCAACGCTGTGACCGAGAGCGAGAAGGCACTGCAGAGCAACCACTTTGAAC
TGAGCCTGCGCACTGAGGCCACGCAGGGGCTGGTGCTCTGGAGTGGCAAGGCCACGGAGCGGGCAGAC
TATGTGGCACTGGCCATTGTGGACGGGCACCTGCAACTGAGCTACAACCTGGGCTCCCAGCCCGTGGT
GCTGCGTTCCACCGTGCCCGTCAACACCAACCGCTGGTTGCGGGTCGTGGCACATAGGGAGCAGAGGG
AAGGTTCCCTGCAGGTGGGCAATGAGGCCCCTGTGACCGGCTCCTCCCCGCTGGGCGCCACGCAGCTG
GACACTGATGGAGCCCTGTGGCTTGGGGGCCTGCCGGAGCTGCCCGTGGGCCCAGCACTGCCCAAGGC
CTACGGCACAGGCTTTGTGGGCTGCTTGCGGGATGTGGTGGTGGGCCGGCACCCGCTGCACCTGCTGG
AGGACGCCGTCACCAAGCCAGAGCTGCGGCCCTGCCCCACCCCATGA
CCGCT

| | | | |
|---|---|---|---|
| NOV60g, CG94946-05 | SEQ ID NO: 1186 | 1544 aa | MW at 162003.7kD |
| Protein Sequence | | | |

MRHGRPVPPGPAAGRPLLPLLVVAACVLPGAGGTCPERAIERREEEAINVLTGTVEEILNVDPVQHTY
SCKVRVWRYLKGKDLVARESLLDGGNKVVISGFGDPLICDNQVSTGDTRIFFVNPAPPYLWPAHKNEL
MLNSSLMRITLRNLEEVEFCVEDKPGTHFTPVPPTPPDACRGMLCGFGAVCEPNAEGPGPASCVCKKS
PCPSVVAPVCGSDASTYSNECELQRAQCSQQRRIRLLSRGPCGSRDPCSNVTCSFGSTCARSADGLTA
SCLCPATCRGAPEGTVCGSDGADYPGECQLLRRACARQENVFKKFDGPCDPCQGALPDPSRSCRVNPR
TRRPEMLLRPESCPARQAPVCGDDGVTYENDCVMGRSGAARGLLLQKVRSGQCQGRDQCPEPCRFNAV
CLSRRGRPRCSCDRVTCDGAYRPVCAQDGRTYDSDCWRQQAECRQQRAIPSKHQGPCDQAPSPCLGVQ
CAFGATCAVKNGQAACECLQACSSLYDPVCGSDGVTYGSACELEATACTLGREIQVARKGPCCRCGQC
RFGALCEAETGRCVCPSECVALAQPVCGSDGHTYPSECMLHVHACTHQISLHVASAGPCETCGDAVCA
FGAVCSAGQCVCPRCEHPPPGPVCGSDGVTYGSACELREAACLQQTQIEEARAGPCEQAECGSGGSGS
GEDGDCEQELCRQRGGIWDEDSEDGPCVCDFSCQSVPGSPVCGSDGVTYSTECELKKARCESQRGLYV
AAQGACRGPAFAPLPPVAPLHCAQTPYGCCQDNITAARGVGLAGCPSACQCNPHGSYGGTCDPATGQC
SCRPGVGGLRCDRCEPGFWNFRGIVTDGRSGCTPCSCDPQGAVRDDCEQMTGLCSCKPGVAGPKCGQC
PDGRALGPAGCEADASAPATCAEMRCEFGARCVEESGSAHCVCPMLTCPEANATKVCGSDGVTYGNEC
QLKTIACRQGLQISIQSLGPCQEAVAPSTHPTSASVTVTTPGLLLSQALPAPPGALPLAPSSTAHSQT
TPPPSSRPRTTASVPRTTVWPVLTVPPTAPSPAPSLVASAFGESGSTDGSSDEELSGDQEASGGGSGG
LEPLEGSSVATPGPPVERASCYNSALGCCSDGKTPSLDAEGSNCPATKVFQGVLELEGVEGQELFYTP
EMADPKSELFGETARSIESTLDDLFRNSDVKKDFRSVRLRDLGPGKSVRAIVDVHFDPTTAFRAPDVA
RALLRQIQVSRRRSLGVRRPLQEHVRFMDFDWFPAFITGATSGAIAAGATARATTASRLPSSAVTPRA
PHPSHTSQPVAKTTAAPTTRRPPTTAPSRVPGPRPPASCVPREAAYVCLCPGGFSGPHCEKGLVEKSA
GDVDTLAFDGRTFVEYLNAVTESEKALQSNHFELSLRTEATQGLVLWSGKATERADYVALAIVDGHLQ
LSYNLGSQPVVLRSTVPVNTNRWLRVVAHREQREGSLQVGNEAPVTGSSPLGATQLDTDGALWLGGLP
ELPVGPALPKAYGTGFVGCLRDVVVGRHPLHLLEDAVTKPELRPCPTP

| | | |
|---|---|---|
| NOV60h, CG94946-06 | SEQ ID NO: 1187 | 6494 bp |
| DNA Sequence | ORF Start: ATG at 37 | ORF Stop: TGA at 6466 |

CCGGCGCGGCCCGCGCGCTCTTCCGCCGCCTCTCGCATGCGCCATGGCCGGCCGGTCCCACCCGGGCC
CGCTGCGGGGCGGCCGCTGCTGCCTCTCCTTGTGGTGGCCGCGTGCGTCCTGCCCGGAGCCGGCGGGA
CATGCCCGGAGCGCGCGCTGGAGCGGCGCGAGGAGGAGGCGAACGTGGTGCTCACCGGGACGGTGGAG
GAGATCCTCAACGTGGACCCGGTGCAGCACACGTACTCCTGCAAGGTTCGGGTCTGGCGGTACTTGAA
GGGCAAAGACCTGGTGGCCCGGGAGAGCCTGCTGGACGGCGGCAACAAGGTGGTGATCAGCGGCTTTG
GAGACCCCCTCATCTGTGACAACCAGGTGTCCACTGGGGACACCAGGATCTTCTTTGTGAACCCTGCA
CCCCCATACCTGTGGCCAGCCCACAAGAACGAGCTGATGCTCAACTCCAGCCTCATGCGGATCACCCT
GCGGAACCTGGAGGAGGTGGAGTTCTGTGTGGAAGATAAACCCGGGACCCACTTCACTCCAGTGCCTC
CGACGCCTCCTGATGCGTGCCGGGGAATGCTGTGCGGCTTCGGCGCCGTGTGCGAGCCCAACGCGGAG
GGGCCGGGCCGGGCGTCCTGCGTCTGCAAGAAGAGCCCGTGCCCCAGCGTGGTGGCGCCTGTGTGTGG
GTCGGACGCCTCCACCTACAGCAACGAATGCGAGCTGCAGCGGGCGCAGTGCAGCCAGCAGCGCCGCA
TCCGCCTGCTCAGCCGCGGGCCGTGCGGCTCGCGGGACCCCTGCTCCAACGTGACCTGCAGCTTCGGC
AGCACCTGTGCGCGCTCGGCCGACGGGCTGACGGCCTCGTGCCTGTGCCCCGCGACCTGCCGTGGCGC
CCCCGAGGGGACCGTCTGCGGCAGCGACGGCGCCGACTACCCCGGCGAGTGCCAGCTCCTGCGCCGCG
CCTGCGCCCGCCAGGAGAATGTCTTCAAGAAGTTCGACGGCCCTTGTGACCCCTGTCAGGGCGCCCTC
CCTGACCCGAGCCGCAGCTGCCGTGTGAACCCGCGCACGCGGCGCCCTGAGATGCTCCTACGGCCCGA
GAGCTGCCCTGCCCGGCAGGCGCCAGTGTGTGGGGACGACGGAGTCACCTACGAAAACGACTGTGTCA
TGGGCCGATCGGGGGCCGCCCGGGGTCTCCTCCTGCAGAAAGTGCGCTCCGGCCAGTGCCAGGGTCGA
GACCAGTGCCCGGAGCCCTGCCGGTTCAATGCCGTGTGCCTGTCCCGCCGTGGCCGTCCCCGCTGCTC
CTGCGACCGCGTCACCTGTGACGGGGCCTACAGGCCCGTGTGTGCCCAGGACGGGCGCACGTATGACA
GTGATTGCTGGCGGCAGCAGGCTGAGTGCCGGCAGCAGCGTGCCATCCCCAGCAAGCACCAGGGCCCG
TGTGACCAGGCCCCGTCCCCATGCCTCGGGGTGCAGTGTGCATTTGGGGCGACGTGTGCTGTGAAGAA
CGGGCAGGCAGCGTGTGAATGCCTGCAGGCGTGCTCGAGCCTCTACGATCCTGTGTGCGGCAGCGACG
GCGTCACATACGGCAGCGCGTGCGAGCTGGAGGCCACGGCCTGTACCCTCGGGCGGGAGATCCAGGTG
GCGCGCAAAGGACCCTGTGACCGCTGCGGGCAGTGCCGCTTTGGAGCCCTGTGCGAGGCCGAGACCGG
GCGCTGCGTGTGCCCCTCTGAATGCGTGGCTTTGGCCCAGCCCGTGTGTGGCTCCGACGGGCACACGT
ACCCCAGCGAGTGCATGCTGCACGTGCACGCCTGCACACACCAGATCAGCCTGCACGTGGCCTCAGCT
GGACCCTGTGAGACCTGTGGAGATGCCGTGTGTGCTTTTGGGGCTGTGTGCTCCGCAGGGCAGTGTGT
GTGTCCCCGGTGTGAGCACCCCCCGCCCGGCCCCGTGTGTGGCAGCGACGGTGTCACCTACGGCAGTG
CCTGCGAGCTACGGGAAGCCGCCTGCCTCCAGCAGACACAGATCGAGGAGGCCCGGGCAGGGCCGTGC
GAGCAGGCCGAGTGCGGTTCCGGAGGCTCTGGCTCTGGGGAGGACGGTGACTGTGAGCAGGAGCTGTG
CCGGCAGCGCGGTGGCATCTGGGACGAGGACTCGGAGGACGGGCCGTGTGTCTGTGACTTCAGCTGCC
AGAGTGTCCCAGGCAGCCCGGTGTGCGGCTCAGATGGGGTCACCTACAGCACCGAGTGTGAGCTGAAG
AAGGCCAGGTGTGAGTCACAGCGAGGGCTCTACGTAGCGGCCCAGGGAGCCTGCCGAGGCCCCGCCTT
CGCCCCGCTGCCGCCTGTGGCCCCCTTACACTGTGCCCAGACGCCCTACGGCTGCTGCCAGGACAATA
TCACCGCAGCCCGGGGCGTGGGCCTGGCTGGCTGCCCCAGTGCCTGCCAGTGCAACCCCCATGGCTCT
TACGGCGGCACCTGTGACCCAGCCACAGGCCAGTGCTCCTGCCGCCCAGGTGTGGGGGGCCTCAGGTG
TGACCGCTGTGAGCCTGGCTTCTGGAACTTTCGAGGCATCGTCACCGATGGCCGGAGTGGCTGTACAC
CCTGCAGCTGTGATCCCCAAGGCGCCGTGCGGGATGACTGTGAGCAGATGACGGGGCTGTGCTCGTGT
AAGCCCGGGGTGGCTGGACCCAAGTGTGGGCAGTGTCCAGACGGCCGTGCCCTGGGCCCCGCGGGCTG
TGAAGCTGACGCTTCTGCGCCTGCGACCTGTGCGGAGATGCGCTGTGAGTTCGGTGCGCGGTGCGTGG
AGGAGTCTGGCTCAGCCCACTGTGTCTGCCCGATGCTCACCTGTCCAGAGGCCAACGCTACCAAGGTC
TGTGGGTCAGATGGAGTCACATACGGCAACGAGTGTCAGCTGAAGACCATCGCCTGCCGCCAGGGCCT
GCAAATCTCTATCCAGAGCCTGGGCCCGTGCCAGGAGGCTGTTGCTCCCAGCACTCACCCGACATCTG
CCTCCGTGACTGTGACCACCCCAGGGCTCCTCCTGAGCCAGGCACTGCCGGCCCCCCCCGGCGCCCTC
CCCCTGGCTCCCAGCAGTACCGCACACAGCCAGACCACCCCTCCGCCCTCATCGCGACCTCGGACCAC
TGCCAGCGTCCCCAGGACCACCGTGTGGCCCGTGCTGACGGTGCCCCCCACGGCACCCTCCCCTGCAC
CCAGCCTGGTGGCGTCCGCCTTTGGTGAATCTGGCAGCACTGATGGAAGCAGCGATGAGGAACTGAGC
GGGGACCAGGAGGCCAGTGGGGGTGGCTCTGGGGGGCTCGAGCCCTTGGAGGGCAGCAGCGTGGCCAC
CCCTGGGCCACCTGTCGAGAGGGCTTCCTGCTACAACTCCGCGTTGGGCTGCTGCTCTGATGGGAAGA
CGCCCTCGCTGGACGCAGAGGGCTCCAACTGCCCCGCCACCAAGGTGTTCCAGGGCGTCCTGGAGCTG
GAGGGCGTCGAGGGCCAGGAGCTGTTCTACACGCCCGAGATGGCTGACCCCAAGTCAGAACTGTTCGG
GGAGACAGCCAGGAGCATTGAGAGCACCCTGGACGACCTCTTCCGGAATTCAGACGTCAAGAAGGATT
TCCGGAGTGTCCGCTTGCGGGACCTGGGGCCCGGCAAATCCGTCCGCGCCATTGTGGATGTGCACTTT
GACCCCACCACAGCCTTCAGGGCACCCGACGTGGCCCGGGCCCTGCTCCGGCAGATCCAGGTGTCCAG
GCGCCGGTCCTTGGGGGTGAGGCGGCCGCTGCAGGAGCACGTGCGATTTATGGACTTTGACTGGTTTC
CTGCGTTTATCACGGGGGCCACGTCAGGAGCCATTGCTGCGGGAGCCACGGCCAGAGCCACCACTGCA
TCGCGCCTGCCGTCCTCTGCTGTGACCCCTCGGGCCCCGCACCCCAGTCACACAAGCCAGCCCGTTGC
CAAGACCACGGCAGCCCCCACCACACGTCGGCCCCCCACCACTGCCCCCAGCCGTGTGCCCGGACGTC
GGCCCCCGGCCCCCCAGCAGCCTCCAAAGCCCTGTGACTCACAGCCCTGCTTCCACGGGGGGACCTGC
CAGGACTGGGCATTGGGCGGGGGCTTCACCTGCAGCTGCCCGGCAGGCAGGGGAGGCGCCGTCTGTGA
GAAGGTGCTTGGCGCCCCTGTGCCGGCCTTCGAGGGCCGCTCCTTCCTGGCCTTCCCCACCCTCCGCG
CCTACCACACGCTGCGCCTGGCACTGGAATTCCGGGCGCTGGAGCCTCAGGGGCTGCTGCTGTACAAT
GGCAACGCCCGGGGCAAGGACTTCCTGGCATTGGCGCTGCTAGATGGCCGCGTGCAGCTCAGGTTTGA
CACAGGTTCGGGGCCGGCGGTGCTGACCAGTGCCGTGCCGGTAGAGCCGGGCCAGTGGCACCGCCTGG
AGCTGTCCCGGCACTGGCGCCGGGGCACCCTCTCGGTGGATGGTGAGACCCCTGTTCTGGGCGAGAGT
CCCAGTGGCACCGACGGCCTCAACCTGGACACAGACCTCTTTGTGGGCGGCGTACCCGAGGACCAGGC
TGCCGTGGCGCTGGAGCGGACCTTCGTGGGCGCCGGCCTGAGGGGGTGCATCCGTTTGCTGGACGTCA
ACAACCAGCGCCTGGAGCTTGGCATTGGGCCGGGGGCTGCCACCCGAGGCTCTGGCGTGGGCGAGTGC
GGGGACCACCCCTGCCTGCCCAACCCCTGCCATGGCGGGGCCCCATGCCAGAACCTGGAGGCTGGAAG
GTTCCATTGCCAGTGCCCGCCCGGCCGCGTCGGACCAACCTGTGCCGATGAGAAGAGCCCCTGCCAGC
CCAACCCCTGCCATGGGGCGGCGCCCTGCCGTGTGCTGCCCGAGGGTGGTGCTCAGTGCGAGTGCCCC
CTGGGGCGTGAGGGCACCTTCTGCCAGACAGCCTCGGGGCAGGACGGCTCTGGGCCCTTCCTGGCTGA
CTTCAACGGCTTCTCCCACCTGGAGCTGAGAGGCCTGCACACCATTGCACGGGACCTGGGGGAGAAGA
TGGCGCTGGAGGCCGTGTTCCTGGCACGAGGCCCCAGCGGCCTCCTGCTCTACAACGGGCAGAAGACG
GACGGCAAGGGGGACTTCGTGTCGCTGGCACTGCGGGACCGCCGCCTGGAGTTCCGCTACGACCTGGG
CAAGGGGGCAGCGGTCATCAGGAGCAGGGAGCCAGTCACCCTGGGAGCCTGGACCAGGGTCTCACTGG
AGCGAAACGGCCGCAAGGGTGCCCTGCGTGTGGGCGACGGCCCCCGTGTGTTGGGGGAGTCCCCGAAA
TCCCGCAAGGTTCCGCACACCGTCCTCAACCTGAAGGAGCCGCTCTACGTAGGGGGCGCTCCCGACTT
CAGCAAGCTGGCCCGTGCTGCTGCCGTGTCCTCTGGCTTCGACGGTGCCATCCAGCTGGTCTCCCTCG
GAGGCCGCCAGCTGCTGACCCCGGAGCACGTGCTGCGGCAGGTGGACGTCACGTCCTTTGCAGGTCAC
CCCTGCACCCGGGCCTCAGGCCACCCCTGCCTCAATGGGGCCTCCTGCGTCCCGAGGGAGGCTGCCTA
TGTGTGCCTGTGTCCCGGGGGATTCTCAGGACCGCACTGCGAGAAGGGGCTGGTGGAGAAGTCAGCGG
GGGACGTGGATACCTTGGCCTTTGACGGGCGGACCTTTGTCGAGTACCTCAACGCTGTGACCGAGAGC
GAGAAGGCACTGCAGAGCAACCACTTTGAACTGAGCCTGCGCACTGAGGCCACGCAGGGGCTGGTGCT
CTGGAGTGGCAAGGCCACGGAGCGGGCAGACTATGTGGCACTGGCCATTGTGGACGGGCACCTGCAAC
TGAGCTACAACCTGGGCTCCCAGCCCGTGGTGCTGCGTTCCACCGTGCCCGTCAACACCAACCGCTGG
TTGCGGGTCGTGGCACATAGGGAGCAGAGGGAAGGTTCCCTGCAGGTGGGCAATGAGGCCCCTGTGAC
CGGCTCCTCCCCGCTGGGCGCCACGCAGCTGGACACTGATGGAGCCCTGTGGCTTGGTGAGTGTTTTG
GGGAGACTAGAGAGGGATGCCCAAGGGTCTCAGATATCCGAGGGACAGACTCCACCCCCCAGCGCCCA
CCCTTGAGTCAGGGTGCATGTGAGCCGGCGGGCTGGGCTCTCTTCTCCCGCTGTAGCCCCTGCAGTTC
CCAGTGCTGTGGGGCCGGGAGGCGGGTGCCCAGGTGTGGGCCCCCTGCTGGTCACCTGCTCGTTGGGG
TGCCCATCAGCATCACTGAGTCACAGCCGGGTGACTCCCACTGTCTGTGCTGCAGGGGCCTGCCGGAG
CTGCCCGTGGGCCCAGCACTGCCCAAGGCCTACGGCACAGGCTTTGTGGGCTGCTTGCGGGATGTGGT
GGTGGGCCGGCACCCGCTGCACCTGCTGGAGGACGCCGTCACCAAGCCAGAGCTGCGGCCCTGCCCCA
CCCCATGAGCTGGCACCAGAGCCCCGCGCCCGCT

| | | | |
|---|---|---|---|
| NOV60h, CG94946-06 | SEQ ID NO: 1188 | 2143 aa | MW at 225054.6kD |
| Protein Sequence | | | |

MRHGRPVPPGPAAGRPLLPLLVVAACVLPGAGGTCPERALERREEEANVVLTGTVEEILNVDPVQHTY
SCKVRVWRYLKGKDLVARESLLDGGNKVVISGFGDPLICDNQVSTGDTRIFFVNPAPPYLWPAHKNEL
MLNSSLMRITLRNLEEVEFCVEDKPGTHFTPVPPTPPDACRGMLCGFGAVCEPNAEGPGRASCVCKKS
PCPSVVAPVCGSDASTYSNECELQRAQCSQQRRIRLLSRGPCGSRDPCSNVTCSFGSTCARSADGLTA
SCLCPATCRGAPEGTVCGSDGADYPGECQLLRPACARQENVFKKFDGPCDPCQGALPDPSRSCRVNPR
TRRPEMLLRPESCPARQAPVCGDDGVTYENDCVMGRSGAARGLLLQKVRSGQCQGRDQCPEPCRFNAV
CLSRRGRPRCSCDRVTCDGAYRPVCAQDGRTYDSDCWRQQAECRQQRAIPSKHQGPCDQAPSPCLGVQ
CAFGATCAVKNGQAACECLQACSSLYDPVCGSDGVTYGSACELEATACTLGREIQVARKGPCDRCGQC
RFGALCEAETGRCVCPSECVALAQPVCGSDGHTYPSECMLHVHACTHQISLHVASAGPCETCGDAVCA
FGAVCSAGQCVCPRCEHPPPGPVCGSDGVTYGSACELREAACLQQTQIEEARAGPCEQAECGSGGSGS
GEDGDCEQELCRQRGGIWDEDSEDGPCVCDFSCQSVPGSPVCGSDGVTYSTECELKKARCESQRGLYV
AAQGACRGPAFAPLPPVAPLHCAQTPYGCCQDNITAARGVGLAGCPSACQCNPHGSYGGTCDPATGQC
SCRPGVGGLRCDRCEPGFWNFRGIVTDGRSGCTPCSCDPQGAVRDDCEQMTGLCSCKPGVAGPKCGQC
PDGRALGPAGCEADASAPATCAEMRCEFGARCVEESGSAHCVCPMLTCPEANATKVCGSDGVTYGNEC
QLKTIACRQGLQISIQSLGPCQEAVAPSTHPTSASVTVTTPGLLLSQALPAPPGALPLAPSSTAHSQT
TPPPSSRPRTTASVPRTTVWPVLTVPPTAPSPAPSLVASAFGESGSTDGSSDEELSGDQEASGGGSGG
LEPLEGSSVATPGPPVERASCYNSALGCCSDGKTPSLDAEGSNCPATKVFQGVLELEGVEGQELFYTP
EMADPKSELFGETARSIESTLDDLFRNSDVKKDFRSVRLRDLGPGKSVRAIVDVHFDPTTAFRAPDVA
RALLRQIQVSRRRSLGVRRPLQENVRFMDFDWFPAFITGATSGAIAAGATARATTASRLPSSAVTPRA
PHPSHTSQPVAKTTAAPTTRRPPTTAPSRVPGRRPPAPQQPPKPCDSQPCFHGGTCQDWALGGGFTCS
CPAGRGGAVCEKVLGAPVPAFEGRSFLAFPTLRAYHTLRLALEFRALEPQGLLLYNGNARGKDFLALA
LLDGRVQLRFDTGSGPAVLTSAVPVEPGQWHRLELSRHWRRGTLSVDGETPVLGESPSGTDGLNLDTD
LFVGGVPEDQAAVALERTFVGAGLRGCIRLLDVNNQRLELGIGPGAATRGSGVGECGDHPCLPNPCHG
GAPCQNLEAGRFHCQCPPGRVGPTCADEKSPCQPNPCHGAAPCRVLPEGGAQCECPLGREGTFCQTAS
GQDGSGPFLADFNGFSHLELRGLHTIARDLGEKMALEAVFLARGPSGLLLYNGQKTDGKGDFVSLALR
DRRIEFRYDLGKGAAVIRSREPVTLGAWTRVSLERNGRKGALRVGDGPRVLGESPKSRKVPHTVLNLK
EPLYVGGAPDFSKLARAAAVSSGFDGAIQLVSLGGRQLLTPEHVLRQVDVTSFAGHPCTRASGHPCLN
GASCVPREAAYVCLCPGGFSGPHCEKGLVEKSAGDVDTLAFDGRTFVEYLNAVTESEKALQSNHFELS
LRTEATQGLVLWSGKATERADYVALAIVDGHLQLSYNLGSQPVVLRSTVPVNTNRWLRVVAHREQREG
SLQVGNEAPVTGSSPLGATQLDTDGALWLGECFGETREGCPRVSDIRGTDSTPQRPPLSQGACEPAGW
ALFSRCSPCSSQCCGAGRRVPRCGPPAGHLLVGVPISITESQPGDSHCLCCRGLPELPVGPALPKAYG
TGFVGCLRDVVVGRHPLHLLEDAVTKPELRPCPTP

| | | |
|---|---|---|
| NOV60i, CG94946-07 | SEQ ID NO: 1189 | 5688 bp |
| DNA Sequence | ORF Start: ATG at 37 | ORF Stop: TGA at 4735 |

CCGGCGCGGCCCGCGCGCTCTTCCGCCGCCTCTCGCATGCGCCATGGCCGGCCGGTCCCACCCGGGCC
CGCTGCGGGGCGGCCGCTGCTGCCTCTCCTTGTGGTGGCCGCGTGCGTCCTGCCCGGAGCCGGCGGGA
CATGCCCGGAGCGCGCGCTGGAGCGGCGCGAGGAGGAGGCGAACGTGGTGCTCACCGGGACGGTGGAG
GAGATCCTCAACGTGGACCCGGTGCAGCACACGTACTCCTGCAAGGTTCGGGTCTGGCGGTACTTGAA
GGGCAAAGACCTGGTGGCCCGGGAGAGCCTGCTGGACGGCGGCAACAAGGTGGTGATCAGCGGCTTTG
GAGACCCCCTCATCTGTGACAACCAGGTGTCCACTGGGGACACCAGGATCTTCTTTGTGAACCCTGCA
CCCCCATACCTGTGGCCAGCCCACAAGAACGAGCTGATGCTCAACTCCAGCCTCATGCGGATCACCCT
GCGGAACCTGGAGGAGGTGGAGTTCTGTGTGGAAGATAAACCCGGGACCCACTTCACTCCAGTGCCTC
CGACGCCTCCTGATGCGTGCCGGGGAATGCTGTGCGGCTTCGGCGCCGTGTGCGAGCCCAACGCGGAG
GGGCCGGGCCGGGCGTCCTGCGTCTGCAAGAAGAGCCCGTGCCCCAGCGTGGTGGCGCCTGTGTGTGG
GTCGGACGCCTCCACCTACAGCAACGAATGCGAGCTGCAGCGGGCGCAGTGCAGCCAGCAGCGCCGCA
TCCGCCTGCTCAGCCGCGGGCCGTGCGGCTCGCGGGACCCCTGCTCCAACGTGACCTGCAGCTTCGGC
AGCACCTGTGCGCGCTCGGCCGACGGGCTGACGGCCTCGTGCCTGTGCCCCGCGACCTGCCGTGGCGC
CCCCGAGGGGACCGTCTGCGGCAGCGACGGCGCCGACTACCCCGGCGAGTGCCAGCTCCTGCGCCGCG
CCTGCGCCCGCCAGGAGAATGTCTTCAAGAAGTTCGACGGCCCTTGTGACCCCTGTCAGGGCGCCCTC
CCTGACCCGAGCCGCAGCTGCCGTGTGAACCCGCGCACGCGGCGCCCTGAGATGCGCCTACGGCCCGA
GAGCTGCCCTGCCCGGCAGGCGCCAGTGTGTGGGGACGACGGAGTCACCTACGAAAACGACTGTGTCA
TGGGCCGATCGGGGGCCGCCCGGGGTCTCCTCCTGCAGAAAGTGCGCTCCGGCCAGTGCCAGGGTCGA
GACCAGTGCCCGGAGCCCTGCCGGTTCAATGCCGTGTGCCTGTCCCGCCGTGGCCGTCCCCGCTGCTC
CTGCGACCGCGTCACCTGTGACGGGGCCTACAGGCCCGTGTGTGCCCAGGACGGGCGCACGTATGACA
GTGATTGCTGGCGGCAGCAGGCTGAGTGCCGGCAGCAGCGTGCCATCCCCAGCAAGCACCAGGGCCCG
TGTGACCAGGCCCCGTCCCCATGCCTCGGGGTGCAGTGTGCATTTGGGGCGACGTGTGCTGTGAAGAA
CGGGCAGGCAGCGTGTGAATGCCTGCAGGCGTGCTCGAGCCTCTACGATCCTGTGTGCGGCAGCGACG
GCGTCACATACGGCAGCGCGTGCGAGCTGGAGGCCACGGCCTGTACCCTCGGGCGGGAGATCCAGGTG
GCGCGCAAAGGACCCTGTGACCGCTGCGGGCAGTGCCGCTTTGGAGCCCTGTGCGAGGCCGAGACCGG
GCGCTGCGTGTGCCCCTCTGAATGCGTGGCTTTGGCCCAGCCCGTGTGTGGCTCCGACGGGCACACGT
ACCCCAGCGAGTGCATGCTGCACGTGCACGCCTGCACACACCAGATCAGCCTGCACGTGGCCTCAGCT
GGACCCTGCGAGACCTGTGGAGATGCCGTGTGTGCTTTTGGGGCTGTGTGCTCCGCAGGGCAGTGTGT
GTGTCCCCGGTGTGAGCACCCCCCGCCCGGCCCCGTGTGTGGCAGCGACGGTGTCACCTACGGCAGTG
CCTGCGAGCTACGGGAAGCCGCCTGCCTCCAGCAGACACAGATCGAGGAGGCCCGGGCAGGGCCGTGC
GAGCAGGCCGAGTGCGGTTCCGGAGGCTCTGGCTCTGGGGAGGACGGTGACTGTGAGCAGGAGCTGTG
CCGGCAGCGCGGTGGCATCTGGGACGAGGACTCGGAGGACGGGCCGTGTGTCTGTGACTTCAGCTGCC
AGAGTGTCCCAGGCAGCCCGGTGTGCGGCTCAGATGGGGTCACCTACAGCACCGAGTGTGAGCTGAAG
AAGGCCAGGTGTGAGTCACAGCGAGGGCTCTACGTAGCGGCCCAGGGAGCCTGCCGAGGCCCCACCTT
CGCCCCGCTGCCGCCTGTGGCCCCCTTACACTGTGCCCAGACGCCCTACGGCTGCTGCCAGGACAATA
TCACCGCAGCCCGGGGCGTGGGCCTGGCTGGCTGCCCCAGTGCCTGCCAGTGCAACCCCCATGGCTCT
TACGGCGGCACCTGTGACCCAGCCACAGGCCAGTGCTCCTGCCGCCCAGGTGTGGGGGGCCTCAGGTG
TGACCGCTGTGAGCCTGGCTTCTGGAACTTTCGAGGCATCGTCACCGATGGCCGGAGTGGCTGTACAC
CCTGCAGCTGTGATCCCCAAGGCGCCGTGCGGGATGACTGTGAGCAGATGACGGGGCTGTGCTCGTGT
AAGCCCGGGGTGGCTGGACCCAAGTGTGGGCAGTGTCCAGACGGCCGTGCCCTGGGCCCCGCGGGCTG
TGAAGCTGACGCTTCTGCGCCTGCGACCTGTGCGGAGATGCGCTGTGAGTTCGGTGCGCGGTGCGTGG
AGGAGTCTGGCTCAGCCCACTGTGTCTGCCCGATGCTCACCTGTCCAGAGGCCAACGCTACCAAGGTC
TGTGGGTCAGATGGAGTCACATACGGCAACGAGTGTCAGCTGAAGACCATCGCCTGCCGACGGTGTCA
CCTACGCCAGGGCCTGCAAATCTCTATCCAGAGCCTGGGCCCGTGCCAGGAGGCTGTTGCTCCCAGCA
CTCACCCGACATCTGCCTCCGTGACTGTGACCACCCCAGGGCTCCTCCTGAGCCAGGCACTGCCGGCC
CCCCCCGGCGCCCTCCCCCTGGCTCCCAGCAGTACCGCACACAGCCAGACCACCCCTCCGCCCTCATC
GCGACCTCGGACCACTGCCAGCGTCCCCAGGACCACCGTGTGGCCCGTGCTGACGGTGCCCCCCACGG
CACCCTCCCCTGCACCCAGCCTGGTGGCGTCCGCCTTTGGTGAATCTGGCAGCACTGATGGAAGCAGC
GATGAGGAACTGAGCGGGGACCAGGAGGCCAGTGGGGGTGGCTCTGGGGGGCCCGAGCCCTTGGAGGG
CAGCAGCGTGGCCACCCCTGGGCCACCTGTCGAGAGGGCTTCCTGCTACAACCCCTGCCATGGGGCGG
CGCCCTGCCGTGTGCTGCCCGAGGGTGGTGCTCAGTGCGAGTGCCCCCTGGGGCGTGAGGGCACCTTC
TGCCAGACAGCCTCGGGGCAGGACGGCTCTGGGCCCTTCCTGGCTGACTTCAACGGCTTCTCCCACCT
GGAGCTGAGAGGCCTGCACACCTTTGCACGGGACCTGGGGGAGAAGATGGCGCTGGAGGTCGTGTTCC
TGGCACGAGGCCCCAGCGGCCTCCTGCTCTACAACGGGCAGAAGACGGACGGCAAGGGGGACTTCGTG
TCGCTGGCACTGCGGGACCGCCGCCTGGAGTTCCGCTACGACCTGGGCAAGGGGGCAGCGGTCATCAG
GAGCAGGGAGCCAGTCACCCTGGGAGCCTGGACCAGGGTCTCACTGGAGCGAAACGGCCGCAAGGGTG
CCCTGCGTGTGGGCGACGGCCCCCGTGTGTTGGGGGAGTCCCCGGTTCCGCACACCGTCCTCAACCTG
AAGGAGCCGCTCTACGTAGGGGGCGCTCCCGACTTCAGCAAGCTGGCCCGTGCTGCTGCCGTGTCCTC
TGGCTTCGACGGTGCCATCCAGCTGGTCTCCCTCGGAGGCCGCCAGCTGCTGACCCCGGAGCACGTGC
TGCGGCAGGTGGACGTCACGTCCTTTGCAGGTCACCCCTGCACCCGGGCCTCAGGCCACCCCTGCCTC
AATGGGGCCTCCTGCGTCCCGAGGGAGGCTGCCTATGTGTGCCTGTGTCCCGGGGGATTCTCAGGACC
GCACTGCGAGAAGGGGCTGGTGGAGAAGTCAGCGGGGGACGTGGATACCTTGGCCTTTGACGGGCGGA
CCTTTGTCGAGTACCTCAACGCTGTGACCGAGAGCGAGAAGGCACTGCAGAGCAACCACTTTGAACTG
AGCCTGCGCACTGAGGCCACGCAGGGGCTGGTGCTCTGGAGTGGCAAGGCCACGGAGCGGGCAGACTA
TGTGGCACTGGCCATTGTGGACGGGCACCTGCAACTGAGCTACAACCTGGGCTCCCAGCCCGTGGTGC
TGCGTTCCACCGTGCCCGTCAACACCAACCGCTGGTTGCGGGTCGTGGCACATAGGGAGCAGAGGGAA
GGTTCCCTGCAGGTGGGCAATGAGGCCCCTGTGACCGGCTCCTCCCCGCTGGGCGCCACGCAGCTGGA
CACTGATGGAGCCCTGTGGCTTGGGGGCCTGCCGGAGCTGCCCGTGGGCCCAGCACTGCCCAAGGCCT
ACGGCACAGGCTTTGTGGGCTGCTTGCGGGATGTGGTGGTGGGCCGGCACCCGCTGCACCTGCTGGAG
GACGCCGTCACCAAGCCAGAGCTGCGGCCCTGCCCCACCCCATGAGCTGGCACCAGAGCCCCGCGCCC
GCTGTAATTATTTTCTATTTTTGTAAACTTGTTGCTTTTTGATATGATTTTCTTGCCTGAGTGTTGGC
CGGAGGGACTGCTGGCCCGGCCTCCCTTCCGTCCAGGCAGCCGTGCTGCAGACAGACCTAGTGCTGAG
GGATGGACAGGCGAGGTGGCAGCGTGGAGGGCTCGGCGTGGATGGCAGCCTCAGGACACACACCCCTG
CCTCAAGGTGCTGAGCCCCCGCCTTGCACTGCGCCTGCCCCACGGTGTCCCCGCCGGGAAGCAGCCCC
GGCTCCTGAATCACCCTCGCTCCGTCAGGCGGGACTCGTGTCCCAAAAAGGAAGGGGCTGCTGAGGTC
TGATGGGGCCCTTCCTCCGGGTGACCCCACAGGGCCTTTCCAAGCCCCTATTTGAGCTGCTCCTTCCT
GTGTGTGCTCTGGACCCTGCCTCGGCCTCCTGCGCCAATACTGTGACTTCCAAACAATGTTACTGCTG
GGCACAGCTCTGCGTTGCTCCCGTGCTGCCTGCGCCAGCCCCAGGCTGCTGAGGAGCAGAGGCCAGAC
CAGGGCCGATCTGGGTGTCCTGACCCTCAGCTGGCCCTGCCCAGCCACCCTGGACATGACCGTATCCC
TCTGCCACACCCCAGGCCCTGCGAGGGGCTATCGAGAGGAGCTCACTGTGGGATGGGGTTGACCTCTG
CCGCCTGCCTGGGTATCTGGGCCTGGCCATGGCTGTGTTCTTCATGTGTTGATTTTATTTGACCCCTG
GAGTGGTGGGTCTCATCTTTCCCATCTCGCCTGAGAGCGGCTGAGGGCTGCCTCACTGCAAATCCTCC
CCACAGCGTCAGTGAAAGTCGTCCTTGTCTCAGAATGACCAGGGGCCAGCCAGTGTCTGACCAAGGTC
AAGGGGCAGGTGCAGAGGTGGCAGGGATGGCTCCGAAGCCAGAA

| | | | |
|---|---|---|---|
| NOV60i, CG94946-07 | SEQ ID NO: 1190 | 1566 aa | MW at 164102.4kD |
| Protein Sequence | | | |

MRHGRPVPPGPAAGRPLLPLLVVAACVLPGAGGTCPERALERREEEANVVLTGTVEEILNVDPVQHTY
SCKVRVWRYLKGKDLVARESLLDGGNKVVISGFGDPLICDNQVSTGDTRIFFVNPAPPYLWPAHKNEL
MLNSSLMRITLRNLEEVEFCVEDKPGTHFTPVPPTPPDACRGMLCGFGAVCEPNAEGPGRASCVCKKS
PCPSVVAPVCGSDASTYSNECELQRAQCSQQRRIRLLSRGPCGSRDPCSNVTCSFGSTCARSADGLTA
SCLCPATCRGAPEGTVCGSDGADYPGECQLLRRACARQENVFKKFDGPCDPCQGALPDPSRSCRVNPR
TRRPEMRLRPESCPARQAPVCGDDGVTYENDCVMGRSGAARGLLLQKVRSGQCQGRDQCPEPCRFNAV
CLSRRGRPRCSCDRVTCDGAYRPVCAQDGRTYDSDCWRQQAECRQQRAIPSKHQGPCDQAPSPCLGVQ
CAFGATCAVKNGQAACECLQACSSLYDPVCGSDGVTYGSACELEATACTLGREIQVARKGPCDRCGQC
RFGALCEAETGRCVCPSECVALAQPVCGSDGHTYPSECMLHVHACTHQISLHVASAGPCETCGDAVCA
FGAVCSAGQCVCPRCEHPPPGPVCGSDGVTYGSACELREAACLQQTQIEEARAGPCEQAECGSGGSGS
GEDGDCEQELCRQRGGIWDEDSEDGPCVCDFSCQSVPGSPVCGSDGVTYSTECELKKARCESQRGLYV
AAQGACRGPTFAPLPPVAPLHCAQTPYGCCQDNITAARGVGLAGCPSACQCNPHGSYGGTCDPATGQC
SCRPGVGGLRCDRCEPGFWNFRGIVTDGRSGCTPCSCDPQGAVRDDCEQMTGLCSCKPGVAGPKCGQC
PDGRALGPAGCEADASAPATCAEMRCEFGARCVEESGSAHCVCPMLTCPEANATKVCGSDGVTYGNEC
QLKTIACRRCHLRQGLQISIQSLGPCQEAVAPSTHPTSASVTVTTPGLLLSQALPAPPGALPLAPSST
AHSQTTPPPSSRPRTTASVPRTTVWPVLTVPPTAPSPAPSLVASAFGESGSTDGSSDEELSGDQEASG
GGSGGPEPLEGSSVATPGPPVERASCYNPCHGAAPCRVLPEGGAQCECPLGREGTFCQTASGQDGSGP
FLADFNGFSHLELRGLHTFARDLGEKMALEVVFLARGPSGLLLYNGQKTDGKGDFVSLALRDRRLEFR
YDLGKGAAVIRSREPVTLGAWTRVSLERNGRKGALRVGDGPRVLGESPVPHTVLNLKEPLYVGGAPDF
SKLARAAAVSSGFDGAIQLVSLGGRQLLTPEHVLRQVDVTSFAGHPCTRASGHPCLNGASCVPREAAY
VCLCPGGFSGPHCEKGLVEKSAGDVDTLAFDGRTFVEYLNAVTESEKALQSNHFELSLRTEATQGLVL
WSGKATERADYVALAIVDGHLQLSYNLGSQPVVLRSTVPVNTNRWLRVVAHREQREGSLQVGNEAPVT
GSSPLGATQLDTDGALWLGGLPELPVGPALPKAYGTGFVGCLRDVVVGRHPLHLLEDAVTKPELRPCP
TP

A ClustalW comparison of the above protein sequences yields the following sequence alignment shown in Table 60B.

TABLE 60B
Comparison of the NOV60 protein sequences.

NOV60a ------------------------------------------------------------
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g MRHGRPVPPGPAAGRPLLPLLVVAACVLPGAGGTCPERALERREEEAINVLTGTVEEILN
NOV60h ------------------------------------------------------------
NOV60i ------------------------------------------------------------
NOV60a ------------------------------------------------------------
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g VDPVQHTYSCKVRVWRYLKGKDLVARESLLDGGNKVVISGFGDPLICDNQVSTGDTRIFF
NOV60h ------------------------------------------------------------
NOV60i ------------------------------------------------------------
NOV60a ------------------------------------------------------------
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g VNPAPPYLWPAHKNELMLNSSLNRITLRNLEEVEFCVEDKPGTHFTPVPPTPPDACRGML
NOV60h ------------------------------------------------------------
NOV60i ------------------------------------------------------------
NOV60a ------------------------------------------------------------
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g CGFGAVCEPNAEGPGRASCVCKKSPCPSVVAPVCGSDASTYSNECELQRAQCSQQRRIRL
NOV60h ------------------------------------------------------------
NOV60i ------------------------------------------------------------
NOV60a ------------------------------------------------------------
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g LSRGPCGSRDPCSNVTCSFGSTCARSADGLTASCLCPATCRGAPEGTVCGSDGADYPGEC
NOV60h ------------------------------------------------------------
NOV60i ------------------------------------------------------------
NOV60a ------------------------------------------------------------
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g QLLRPACARQENVFKKFDGPCDPCQGALPDPSRSCRVNPRTRRPEMLLRPESCPARQAPV
NOV60h ------------------------------------------------------------
NOV60i ------------------------------------------------------------
NOV60a ------------------------------------------------------------
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g CGDDGVTYENDCVMGRSGAARGLLLQKVRSGQCQGRDQCPEPCRFNAVCLSRRGRPRCSC
NOV60h ------------------------------------------------------------
NOV60i ------------------------------------------------------------
NOV60a ------------------------------------------------------------
NOV60b ------------------------------------------------------------
NOV60c ------------------------------------------------------------
NOV60d ----------------------MRHGRPVPPGPAAGRPLLPLLVVAACVLPGAGGTCPER
NOV60e ------------------------------------------------------------
NOV60f ------------------------------------------------------------
NOV60g DRVTCDGAYRPVCAQDGRTYDSDCWRQQAECRQQRAIPSKHQGPCDQAPSPCLGVQCAFG
NOV60h ------------------------------------------------------------
NOV60i ----------------------MRHGRPVPPGPAAGRPLLPLLVVAACVLPGAGGTCPER
NOV60a ------------------------------------------------------------
NOV60b ------------------------------------------------------------
NOV60c ------------------------------------------------------------
NOV60d ALERREEEANVVLTGTVEEILNVDPVQHTYSCKVRVWRYLKGKDLVARESLLDGGNKVVI
NOV60e ------------------------------------------------------------
NOV60f ------------------------------------------------------------
NOV60g ATCAVKNGQAACECLQACSSLYDPVCGSDGVTYGSACELEATACTLGREIQVARKGPCDR
NOV60h ------------------------------------------------------------
NOV60i ALERREEEAIWVLTGTVEEILNVDPVQHTYSCKVRVWRYLKGKDLVARESLLDGGNKVVI
NOV60a ------------------------------------------------------------
NOV60b ------------------------------------------------------------
NOV60c ------------------------------------------------------------
NOV60d SGFGDPLICDNQVSTGDTRIFFVNPAPPYLWPAHKNELMLNSSLMRITLRNLEEVEFCVE
NOV60e ------------------------------------------------------------
NOV60f ------------------------------------------------------------
NOV60g CGQCRFGALCEAETGRCVCPSECVALAQPVCGSDGHTYPSECMLHVHACTHQISLHVASA
NOV60h ------------------------------------------------------------
NOV60i SGFGDPLICDNQVSTGDTRIFFVNPAPPYLWPAHKNELMLNSSLMRITLRNLEEVEFCVE
NOV60a ------------------------------------------------------------
NOV60b ------------------------------------------------------------
NOV60c ------------------------------------------------------------
NOV60d DKPGTHFTPVPPTPPDACRGMLCGFGAVCEPNAEGPGRASCVCKKSPCPSVVAPVCGSDA
NOV60e ------------------------------------------------------------
NOV60f ------------------------------------------------------------
NOV60g GPCETCGDAVCAFGAVCSAGQCVCPRCEHPPPGPVCGSDGVTYGSACELREAACLQQTQI
NOV60h ------------------------------------------------------------
NOV60i DKPGTHFTPVPPTPPDACRGMLCGFGAVCEPNAEGPGRASCVCKKSPCPSVVAPVCGSDA
NOV60a ------------------------------------------------------------
NOV60b ------------------------------------------------------------
NOV60c ------------------------------------------------------------
NOV60d STYSNECELQRAQCSQQRRIRLLSRGPCGSRDPCSNVTCSFGSTCARSADGLTASCLCPA
NOV60e ------------------------------------------------------------
NOV60f ------------------------------------------------------------
NOV60g EEARAGPCEQAECGSGGSGSGEDGDCEQELCRQRGGIWDEDSEDGPCVCDFSCQSVPGSP
NOV60h ------------------------------------------------------------
NOV60i STYSNECELQRAQCSQQRRIRLLSRGPCGSRDPCSNVTCSFGSTCARSADGLTASCLCPA
NOV60a ------------------------------------------------------------
NOV60b ------------------------------------------------------------
NOV60c ------------------------------------------------------------
NOV60d TCRGAPEGTVCGSDGADYPGECQLLREACARQENVFKKFDGPCDPCQGALPDPSRSCRVN
NOV60e ------------------------------------------------------------
NOV60f ------------------------------------------------------------
NOV60g VCGSDGVTYSTECELKKARCESQRGLYVAAQGACRGPAFAPLPPVAPLHCAQTPYGCCQD
NOV60h ------------------------------------------------------------
NOV60i TCRGAPEGTVCGSDGADYPGECQLLRRACARQENVFKKFDGPCDPCQGALPDPSRSCRVN
NOV60a ------------------------------------------------------------
NOV60b ------------------------------------------------------------
NOV60c ------------------------------------------------------------
NOV60d PRTRRPEMLLRPESCPARQAPVCGDDGVTYENDCVMGRSGAARGLLLQKVRSGQCQGRDQ
NOV60e ------------------------------------------------------------
NOV60f ------------------------------------------------------------
NOV60g NITAARGVGLAGCPSACQCNPHGSYGGTCDPATGQCSCRPGVGGLRCDRCEPGFWNFRGI
NOV60h ------------------------------------------------------------
NOV60i PRTRRPEMRLRPESCPARQAPVCGDDGVTYENDCVMGRSGAARGLLLQKVRSGQCQGRDQ
NOV60a ------------------------------------------------------------
NOV60b ------------------------------------------------------------
NOV60c ------------------------------------------------------------
NOV60d CPEPCRFNAVCLSRRGRPRCSCDRVTCDGAYRPVCAQDGRTYDSDCWRQQAECRQQRAIP
NOV60e ------------------------------------------------------------
NOV60f ------------------------------------------------------------
NOV60g VTDGRSGCTPCSCDPQGAVRDDCEQMTGLCSCKPGVAGPKCGQCPDGRALGPAGCEADAS
NOV60h ------------------------------------------------------------
NOV60i CPEPCRFNAVCLSRRGRPRCSCDRVTCDGAYRPVCAQDGRTYDSDCWRQQAECRQQRAIP
NOV60a ------------------------------------------------------------
NOV60b ------------------------------------------------------------
NOV60c ------------------------------------------------------------
NOV60d SKHQGPCDQAPSPCLGVQCAFGATCAVKNGQAACECLQACSSLYDPVCGSDGVTYGSACE
NOV60e ------------------------------------------------------------
NOV60f ------------------------------------------------------------
NOV60g APATCAEMRCEFGARCVEESGSAHCVCPMLTCPEANATKVCGSDGVTYGNECQLKTIACR
NOV60h ------------------------------------------------------------
NOV60i SKHQGPCDQAPSPCLGVQCAFGATCAVKNGQAACECLQACSSLYDPVCGSDGVTYGSACE
NOV60a ------------------------------------------------------------
NOV60b ------------------------------------------------------------
NOV60c ------------------------------------------------------------
NOV60d LEATACTLGREIQVARKGPCDRCGQCRFGALCEAETGRCVCPSECVALAQPVCGSDGHTY
NOV60e ------------------------------------------------------------
NOV60f ------------------------------------------------------------
NOV60g QGLQISIQSLGPCQEAVAPSTHPTSASVTVTTPGLLLSQALPAPPGALPLAPSSTAHSQT
NOV60h ------------------------------------------------------------
NOV60i LEATACTLGREIQVARKGPCDRCGQCRFGALCEAETGRCVCPSECVALAQPVCGSDGHTY
NOV60a --------------------------------MRHGRPVPPGPAAGRPLLPLLVVAACVL
NOV60b ------------------------------------------------------------
NOV60c ------------------------------------------------------------
NOV60d PSECMLHVHACTHQISLHVASAGPCETCGDAVCAFGAVCSAGQCVCPRCEHPPPGPVCGS
NOV60e --------------------------------MRHGRPVPPGPAAGRPLLPLLVVAACVL
NOV60f --------------------------------MRHGRPVPPGPAAGRPLLPLLVVAACVL
NOV60g TPPPSSRPRTTASVPRTTVWPVLTVPPTAPSPAPSLVASAFGESGSTDGSSDEELSGDQE
NOV60h --------------------------------MRHGRPVPPGPAAGRPLLPLLVVAACVL
NOV60i PSECMLHVHACTHQISLHVASAGPCETCGDAVCAFGAVCSAGQCVCPRCEHPPPGPVCGS
NOV60a PGAGGTCPERALERREEEANVVLTGTVEEILNVDPVQHTYSCKVRVWRYLKGKDLVARES
NOV60b --------------------------------------------------TGSATKVFQG
NOV60c ------------------------------------------------------------
NOV60d DGVTYGSACELREAACLQQTQIEEARAGPCEQAECGSGGSGSGEDGDCEQELCRQRGGIW
NOV60e PGAGGTCPERALERREEEANVVLTGTVEEILNVDPVQHTYSCKVRVWRYLKGKDLVARES
NOV60f PGAGGTCPERALERREEEANVVLTGTVEEILNVDPVQHTYSCKVRVWRYLKGKDLVARES
NOV60g ASGGGSGGLEPLEGSSVATPGPPVERASCYNSALGCCSDGKTPSLDAEGSNCPATKVFQG
NOV60h PGAGGTCPERALERREEEANVVLTGTVEEILNVDPVQHTYSCKVRVWRYLKGKDLVARES
NOV60i DGVTYGSACELREAACLQQTQIEEARAGPCEQAECGSGGSGSGEDGDCEQELCRQRGGIW
NOV60a LLDGGNKVVISGFGDPLICDNQVSTGDTRIFFVNPAPPYLWPAHKNELMLNSSLMRITLR
NOV60b VLELEGVEGQELFYTPEMADPKSELFGETARSIESTLDDLFRNSDVKKDFRSVRLRDLG-
NOV60c ------------------------------------------------------------
NOV60d DEDSEDGPCVCDFSCQSVPGSPVCGSDGVTYSTECELKKARCESQRGLYVAAQGACRGPA
NOV60e LLDGGNKVVISGFGDPLICDNQVSTGDTRIFFVNPAPPYLWPAHKNELVLWSG-------
NOV60f LLDGGNKVVISGFGDPLICDNQVSTGDTRIFFVNPAPPYLWPAHKNELMLNSSLMRITLR
NOV60g VLELEGVEGQELFYTPEMADPKSELFGETARSIESTLDDLFRNSDVKKDFRSVRLRDLG-
NOV60h LLDGGNKVVISGFGDPLICDNQVSTGDTRIFFVNPAPPYLWPAHKNELMLNSSLMRITLR
NOV60i DEDSEDGPCVCDFSCQSVPGSPVCGSDGVTYSTECELKKARCESQRGLYVAAQGACRGPT
NOV60a NLEEVEFCVEDKPGTHFTPVPPTPPDACRGMLCGFGAVCEPNAEGPGRASCVCKKSPCPS
NOV60b --------------------------------PGKSVRAIVDVHFDP-------------
NOV60c -----------------------------------TGSCQCNPHG---------------
NOV60d FAPLPPVAPLHCAQTPYGCCQDNITAARGVGLAGCPSACQCNPHG---------------
NOV60e ---------------K------------ATERADYVALAIVDGHLQ--------------
NOV60f NLEEVEFCVEDKPGTHFTPVPPTPPDACRGMLCGFGAVCEPNAEGPGRASCVCKKSPCPS
NOV60g --------------------------------PGKSVRAIVDVHFDP-------------
NOV60h NLEEVEFCVEDKPGTHFTPVPPTPPDACRGMLCGFGAVCEPNAEGPGRASCVCKKSPCPS
NOV60i FAPLPPVAPLHCAQTPYGCCQDNITAARGVGLAGCPSACQCNPHG---------------
NOV60a VVAPVCGSDASTYSNECELQRAQCSQQRRIRLLSRGPCGSRDPCSNVTCSFGSTCARSAD
NOV60b ----------TTAFRAPDVARALLRQIQVS------------------------------
NOV60c -----------SYGGTCDPATGQCS-----------------------------------
NOV60d -----------SYGGTCDPATGQCS-----------------------------------
NOV60e ----------LSYNLGSQPVVLRST-----------------------------------
NOV60f VVAPVCGSDASTYSNECELQRAQCSQQRRIRLLSRGPCGSRDPCSNVTCSFGSTCARSAD
NOV60g ----------TTAFRAPDVARALLRQIQVS------------------------------
NOV60h VVAPVCGSDASTYSNECELQRAQCSQQRRIRLLSRGPCGSRDPCSNVTCSFGSTCARSAD
NOV60i -----------SYGGTCDPATGQCS-----------------------------------
NOV60a GLTASCLCPATCRGAPEGTVCGSDGADYPGECQLLRPACARQENVFKKFDGPCDPCQGAL
NOV60b ------------RRRSLGVRRP-----------------LQEHVRFMDFDWVDG------
NOV60c -----------CRPGVGGLRCD------------------RCEPGFWNFRG---------
NOV60d -----------CRPGVGGLRCD------------------RCEPGFWNFRG---------
NOV60e -------------VPVNTNRWL------------------RVVAHREQREG---------
NOV60f GLTASCLCPATCRGAPEGTVCGSDGADYPGECQLLRRACARQENVFKKFDGPCDPCQGAL
NOV60g ------------RRRSLGVRRP-----------------LQEHVRFMDFDWFPAFITGAT
NOV60h GLTASCLCPATCRGAPEGTVCGSDGADYPGECQLLRRACARQENVFKKFDGPCDPCQGAL
NOV60i -----------CRPGVGGLRCD------------------RCEPGFWNFRG---------
NOV60a PDPSRSCRVNPRTRRPEMLLRPESCPARQAPVCGDDGVTYENDCVMGRSGAARGLLLQKV
NOV60b ------------------------------------------------------------
NOV60c -------------------------------IVTD-----------GRS-----------
NOV60d -------------------------------IVTD-----------GRS-----------
NOV60e -----------------------------SLQVGN-------------------------
NOV60f PDPSRSCRVNPRTRRPEMLLRPESCPARQAPVCGDDGVTYENDCVMGRSGAARGLLLQKV
NOV60g SGAIAAGATARATTASRLPSSAVTPRAPHPSHTSQPVAKTTAAPTTRRPPTTAPSRVPGR
NOV60h PDPSRSCRVNPRTRRPEMLLRPESCPARQAPVCGDDGVTYENDCVMGRSGAARGLLLQKV
NOV60i -------------------------------IVTD-----------GRS-----------
NOV60a RSGQCQGRDQCPEPCRFNAVCLSRRGRPRCSCDRVTCDGAYRPVCAQDGRTYDSDCWRQQ
NOV60b ------------------------------------------------------------
NOV60c -------------------------GCTPCSCD---PQGAVRDDCEQ-------------
NOV60d -------------------------GCTPCSCD---PQGAVRDDCEQ-------------
NOV60e---------------------------EAPVTGS--SPLGATQLDTDG-------------
NOV60f RSGQCQGRDQCPEPCRFNAVCLSRRGRPRCSCDRVTCDGAYRPVCAQDGRTYDSDCWRQQ
NOV60g RPPASCVPREAAYVCLCPGGFSGPHCEKGLVEKSAGDVDTLAFDGRTFVEYLNAVTESEK
NOV60h RSGQCQGRDQCPEPCRFNAVCLSRRGRPRCSCDRVTCDGAYRPVCAQDGRTYDSDCWRQQ
NOV60i -------------------------GCTPCSCD---PQGAVRDDCEQ-------------
NOV60a AECRQQRAIPSKHQGPCDQAPSPCLGVQCAFGATCAVKNGQAACECLQACSSLYDPVCGS
NOV60b ------------------------------------------------------------
NOV60c ------------------------------MTGLCSCKPG----------------VAG-
NOV60d ------------------------------MTGLCSCKPG----------------VAG-
NOV60e ---------------------------ALWLGGLPELPVG--------------------
NOV60f AECRQQRAIPSKHQGPCDQAPSPCLGVQCAFGATCAVKNGQAACECLQACSSLYDPVCGS
NOV60g ALQSNHFELSLRTEATQGLVLWSGKATERADYVALAIVDGHLQLSYNLGSQPVVLRSTVP
NOV60h AECRQQRAIPSKHQGPCDQAPSPCLGVQCAFGATCAVKNGQAACECLQACSSLYDPVCGS
NOV60i ------------------------------MTGLCSCKPG----------------VAG-
NOV60a DGVTYGSACELEATACTLGREIQVARKGPCDRCGQCRFGALCEAETGRCVCPSECVALAQ
NOV60b ------------------------------------------------------------
NOV60c ------------------------------PKCGQC------------------------
NOV60d ------------------------------PKCGQC------------------------
NOV60e ------------------------------------------------------------
NOV60f DGVTYGSACELEATACTLGREIQVARKGPCDRCGQCRF----------------------
NOV60g VNTNRWLRVVAHREQREGSLQVGNEAPVTGSSPLGATQLDTDGALWLGGLPELPVGPALP
NOV60h DGVTYGSACELEATACTLGREIQVARKGPCDRCGQCRFGALCEAETGRCVCPSECVALAQ
NOV60i ------------------------------PKCGQC------------------------
NOV60a PVCGSDGHTYPSECMLHVHACTHQISLHVASAGPCETCGDAVCAFGAVCSAGQCVCPRCE
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g KAYGTGFVGCLRDVVVGRHPLHLLEDAVTKPELRPCPTP---------------------
NOV60h PVCGSDGHTYPSECMLHVHACTHQISLHVASAGPCETCGDAVCAFGAVCSAGQCVCPRCE
NOV60i ------------------------------------------------------------
NOV60a HPPPGPVCGSDGVTYGSACELREAACLQQTQIEEARAGPCEQAECGSGGSGSGEDGDCEQ
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g ------------------------------------------------------------
NOV60h HPPPGPVCGSDGVTYGSACELREAACLQQTQIEEARAGPCEQAECGSGGSGSGEDGDCEQ
NOV60i ------------------------------------------------------------
NOV60a ELCRQRGGIWDEDSEDGPCVCDFSCQSVPGSPVCGSDGVTYSTECELKKARCESQRGLYV
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g ------------------------------------------------------------
NOV60h ELCRQRGGIWDEDSEDGPCVCDFSCQSVPGSPVCGSDGVTYSTECELKKARCESQRGLYV
NOV60i ------------------------------------------------------------
NOV60a AAQGACRGPAFAPLPPVAPLHCAQTPYGCCQDNITAARGVGLAGCPSACQCNPHGSYGGT
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g ------------------------------------------------------------
NOV60h AAQGACRGPAFAPLPPVAPLHCAQTPYGCCQDNITAARGVGLAGCPSACQCNPHGSYGGT
NOV60i ------------------------------------------------------------
NOV60a CDPATGQCSCRPGVGGLRCDRCEPGFWNFRGIVTDGRSGCTPCSCDPQGAVRDDCEQMTG
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g ------------------------------------------------------------
NOV60h CDPATGQCSCRPGVGGLRCDRCEPGFWNFRGIVTDGRSGCTPCSCDPQGAVRDDCEQMTG
NOV60i ------------------------------------------------------------
NOV60a LCSCKPGVAGPKCGQCPDGRALGPAGCEADASAPATCAEMRCEFGARCVEESGSAHCVCP
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g ------------------------------------------------------------
NOV60h LCSCKPGVAGPKCGQCPDGRALGPAGCEADASAPATCAEMRCEFGARCVEESGSAHCVCP
NOV60i ------------------------------------------------------------
NOV60a MLTCPEANATKVCGSDGVTYGNECQLKTIACRQGLQISIQSLGPCQEAVAPSTHPTSASV
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g ------------------------------------------------------------
NOV60h MLTCPEANATKVCGSDGVTYGNECQLKTIACRQGLQISIQSLGPCQEAVAPSTHPTSASV
NOV60i ------------------------------------------------------------
NOV60a TVTTPGLLLSQALPAPPGALPLAPSSTAHSQTTPPPSSRPRTTASVPRTTVWPVLTVPPT
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g ------------------------------------------------------------
NOV60h TVTTPGLLLSQALPAPPGALPLAPSSTAHSQTTPPPSSRPRTTASVPRTTVWPVLTVPPT
NOV60i ------------------------------------------------------------
NOV60a APSPAPSLVASAFGESGSTDGSSDEELSGDQEASGGGSGGLEPLEGSSVATPGPPVEEAS
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g ------------------------------------------------------------
NOV60h APSPAPSLVASAFGESGSTDGSSDEELSGDQEASGGGSGGLEPLEGSSVATPGPPVERAS
NOV60i ------------------------------------------------------------
NOV60a CYNSALGCCSDGKTPSLDAEGSNCPATKVFQGVLELEGVEGQELFYTPEMADPKSELFGE
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g ------------------------------------------------------------
NOV60h CYNSALGCCSDGKTPSLDAEGSNCPATKVFQGVLELEGVEGQELFYTPEMADPKSELFGE
NOV60i ------------------------------------------------------------
NOV60a TARSIESTLDDLFRNSDVKKDFRSVRLRDLGPGKSVEAIVDVHFDPTTAFRAPDVAEALL
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g ------------------------------------------------------------
NOV60h TARSIESTLDDLFRNSDVKKDFRSVRLRDLGPGKSVEAIVDVHFDPTTAFRAPDVARALL
NOV60i ------------------------------------------------------------
NOV60a RQIQVSRRRSLGVRRPLQEHVRFMDFDWFPAFITGATSGAIAAGATARATTASRLPSSAV
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g ------------------------------------------------------------
NOV60h RQIQVSRRRSLGVRRPLQEHVRFMDFDWFPAFITGATSGAIAAGATARATTASRLPSSAV
NOV60i ------------------------------------------------------------
NOV60a TPRAPHPSHTSQPVAKTTAAPTTRRPPTTAPSRVPGRRPPAPQQPPKPCDSQPCFHGGTC
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g ------------------------------------------------------------
NOV60h TPRAPHPSHTSQPVAXTTAAPTTRRPPTTAPSRVPGRRPPAPQQPPKPCDSQPCFHGGTC
NOV60i ------------------------------------------------------------
NOV60a QDWALGGGFTCSCPAGRGGAVCEKVLGAPVPAFEGRSFLAFPTLRAYHTLRLALEFRALE
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g ------------------------------------------------------------
NOV60h QDWALGGGFTCSCPAGRGGAVCEKVLGAPVPAFEGRSFLAFPTLRAYHTLRLALEFRALE
NOV60i ------------------------------------------------------------
NOV60a PQGLLLYNGNARGKDFLALALLDGRVQLRFDTGSGPAVLTSAVPVEPGQWHRLELSRHWR
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g ------------------------------------------------------------
NOV60h PQGLLLYNGNARGKDFLALALLDGRVQLRFDTGSGPAVLTSAVPVEPGQWHRLELSRHWR
NOV60i ------------------------------------------------------------
NOV60a RGTLSVDGETPVLGESPSGTDGLNLDTDLFVGGVPEDQAAVALERTFVGAGLRGCIRLLD
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g ------------------------------------------------------------
NOV60h RGTLSVDGETPVLGESPSGTDGLNLDTDLFVGGVPEDQAAVALERTFVGAGLRGCIRLLD
NOV60i ------------------------------------------------------------
NOV60a VNNQRLELGIGPGAATRGSGVGECGDHPCLPNPCHGGAPCQNLEAGRFHCQCPPGRVGPT
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g ------------------------------------------------------------
NOV60h VNNQRLELGIGPGAATRGSGVGECGDHPCLPNPCHGGAPCQNLEAGRFHCQCPPGRVGPT
NOV60i ------------------------------------------------------------
NOV60a CADEKSPCQPNPCHGAAPCRVLPEGGAQCECPLGREGTFCQTASGQDGSGPFLADFNGFS
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g ------------------------------------------------------------
NOV60h CADEKSPCQPNPCHGAAPCRVLPEGGAQCECPLGREGTFCQTASGQDGSGPFLADFNGFS
NOV60i ------------------------------------------------------------
NOV60a HLELRGLHTIARDLGEKMALEAVFLARGPSGLLLYNGQKTDGKGDFVSLALRDRRLEFRY
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g ------------------------------------------------------------
NOV60h HLELRGLHTIARDLGEKMALEAVFLARGPSGLLLYNGQKTDGKGDFVSLALRDRRLEFRY
NOV60i ------------------------------------------------------------
NOV60a DLGKGAAVIRSREPVTLGAWTRVSLERNGRKGALRVGDGPRVLGESPKSRKVPHTVLNLK
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g ------------------------------------------------------------
NOV60h DLGKGAAVIRSREPVTLGAWTRVSLERNGRKGALRVGDGPRVLGESPKSRKVPHTVLNLK
NOV60i ------------------------------------------------------------
NOV60a EPLYVGGAPDFSKLARAAAVSSGFDGAIQLVSLGGRQLLTPEBVLRQVDVTSFAGHPCTR
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g ------------------------------------------------------------
NOV60h EPLYVGGAPDFSKLARAAAVSSGFDGAIQLVSLGGRQLLTPEHVLRQVDVTSFAGHPCTR
NOV60i ------------------------------------------------------------
NOV60a ASGHPCLNGASCVPREAAYSICLCPGGFSGPHCEKGLVEKSAGDVDTLAFDGRTFVEYLNA
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g ------------------------------------------------------------
NOV60h ASGHPCLNGASCVPREAAYVCLCPGGFSGPHCEKGLVEKSAGDVDTLAFDGRTFVEYLNA
NOV60i ------------------------------------------------------------
NOV60a VTESEKALQSNHFELSLRTEATQGLVLWSGKATERADYVALAIVDGHLQLSYNLGSQPVV
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g ------------------------------------------------------------
NOV60h VTESEKALQSNHFELSLRTEATQGLVLWSGKATERADYVALAIVDGHLQLSYNLGSQPVV
NOV60i ------------------------------------------------------------
NOV60a LRSTVPVNTNRWLRVVAHREQREGSLQVGNEAPVTGSSPLGATQLDTDGALWL-------
NOV60b ------------------------------------------------------------
NOV60c ------------------------------------------------------------
NOV60d ------------------------------------------------------------
NOV60e ------------------------------------------------------------
NOV60f -----------------------------GALPVTGSSPLGATQLDTDGALWL-------
NOV60g ------------------------------------------------------------
NOV60h LRSTVPVNTNRWLRVVAHREQREGSLQVGNEAPVTGSSPLGATQLDTDGALWLGECFGET
NOV60i ------------------------------------------------------------
NOV60a ------------------------------------------------------------
NOV60b ------------------------------------------------------------
NOV60c ------------------------------------------------------------
NOV60d ------------------------------------------------------------
NOV60e ------------------------------------------------------------
NOV60f ------------------------------------------------------------
NOV60g ------------------------------------------------------------
NOV60h REGCPRVSDIRGTDSTPQRPPLSQGACEPAGWALFSRCSPCSSQCCGAGRRVPRCGPPAG
NOV60i ------------------------------------------------------------
NOV60a -----------------------GGLPELPVGPALPKAYGTGFVGCLRDVVVGRHPLHLL
NOV60b ------------------------------------------------------------
NOV60c -----------------------------------PDGRALGPAGCVDG-----------
NOV60d -----------------------------------PDGRALGPAGCEADASAPATCAEMR
NOV60e --------------------------------PALPKAYGTGFVGCLRDVVVGRHPLHLL
NOV60f -----------------------GGLPELPVGPALPKAYGTGFVGCLRDVVVGRHPLHLL
NOV60g ------------------------------------------------------------
NOV60h HLLVGVPISITESQPGDSHCLCCRGLPELPVGPALPKAYGTGFVGCLRDVVVGRHPLHLL
NOV60i -----------------------------------PDGRALGPAGCEADASAPATCAENR
NOV60a EDAVTKPELRPCPTP---------------------------------------------
NOV60b ------------------------------------------------------------
NOV60c ------------------------------------------------------------
NOV60d CEFGARCVEESGSAHCVCPMLTCPEANATKVCGSDGVTYGNECQLKTIACRQGLQISIQS
NOV60e EDAVTKPELRPCPTP---------------------------------------------
NOV60f EDAVTKPELRPCPTP---------------------------------------------
NOV60g ------------------------------------------------------------
NOV60h EDAVTKPELRPCPTP---------------------------------------------
NOV60i CEFGARCVEESGSAHCVCPMLTCPEANATKVCGSDGVTYGNECQLKTIACRRCHLRQGLQ
NOV60a ------------------------------------------------------------
NOV60b ------------------------------------------------------------
NOV60c ------------------------------------------------------------
NOV60d LGPCQEAVAPSTHPTSASVTVTTPGLLLSQALPAPPGALPLAPSSTAHSQTTPPPSSRPR
NOV60e ------------------------------------------------------------
NOV60f ------------------------------------------------------------
NOV60g ------------------------------------------------------------
NOV60h ------------------------------------------------------------
NOV60i ISIQSLGPCQEAVAPSTHPTSASVTVTTPGLLLSQALPAPPGALPLAPSSTAHSQTTPPP
NOV60a ------------------------------------------------------------
NOV60b ------------------------------------------------------------
NOV60c ------------------------------------------------------------
NOV60d TTASVPRTTVWPVLTVPPTAPSPAPSLVASAFGESGSTDGSSDEELSGDQEASGGGSGGL
NOV60e ------------------------------------------------------------
NOV60f ------------------------------------------------------------
NOV60g ------------------------------------------------------------
NOV60h ------------------------------------------------------------
NOV60i SSRPRTTASVPRTTVWPVLTVPPTAPSPAPSLVASAFGESGSTDGSSDEELSGDQEASGG
NOV60a ------------------------------------------------------------
NOV60b ------------------------------------------------------------
NOV60c ------------------------------------------------------------
NOV60d EPLEGSSVATPGPPVERASCYNPCHGAAPCRVLPEGGAQCECPLGREGTFCQTASGQDGS
NOV60e ------------------------------------------------------------
NOV60f ------------------------------------------------------------
NOV60g ------------------------------------------------------------
NOV60h ------------------------------------------------------------
NOV60i GSGGPEPLEGSSVATPGPPVERASCYNPCHGAAPCRVLPEGGAQCECPLGREGTFCQTAS
NOV60a ------------------------------------------------------------
NOV60b ------------------------------------------------------------
NOV60c ------------------------------------------------------------
NOV60d GPFLADFNGFSHLELRGLHTIARDLGEKMALEAVFLARGPSGLLLYNGQKTDGKGDFVSL
NOV60e ------------------------------------------------------------
NOV60f ------------------------------------------------------------
NOV60g ------------------------------------------------------------
NOV60h ------------------------------------------------------------
NOV60i GQDGSGPFLADFNGFSHLELRGLHTFARDLGEKMALEVVFLARGPSGLLLYNGQKTDGKG
NOV60a ------------------------------------------------------------
NOV60b ------------------------------------------------------------
NOV60c ------------------------------------------------------------
NOV60d ALRDRRLEFRYDLGKGAAVIRSREPVTLGAWTRVSLERNGRKGALRVGDGPRVLGESPKS
NOV60e ------------------------------------------------------------
NOV60f ------------------------------------------------------------
NOV60g ------------------------------------------------------------
NOV60h ------------------------------------------------------------
NOV60i DFVSLALRDRRLEFRYDLGKGAAVIRSREPVTLGAWTRVSLERNGRKGALRVGDGPRVLG
NOV60a ------------------------------------------------------------
NOV60b ------------------------------------------------------------
NOV60c ------------------------------------------------------------
NOV60d RKVPHTVLNLKEPLYVGGAPDFSKLAPAAAVSSGFDGAIQLVSLGGRQLLTPEHVLRQVD
NOV60e ------------------------------------------------------------
NOV60f ------------------------------------------------------------
NOV60g ------------------------------------------------------------
NOV60h ------------------------------------------------------------
NOV60i ESPVPHTVLNLKEPLYVGGAPDFSKLARAAAVSSGFDGAIQLVSLGGRQLLTPEHVLRQV
NOV60a ------------------------------------------------------------
NOV60b ------------------------------------------------------------
NOV60c ------------------------------------------------------------
NOV60d VTSFAGHPCTPASGHPCLNGASCVPREAAYVCLCPGGFSGPHCEKGLVEKSAGDVDTLAF
NOV60e ------------------------------------------------------------
NOV60f ------------------------------------------------------------
NOV60g ------------------------------------------------------------
NOV60h ------------------------------------------------------------
NOV60i DVTSFAGHPCTRASGHPCLNGASCVPREAAYVCLCPGGFSGPHCEKGLVEKSAGDVDTLA
NOV60a ------------------------------------------------------------
NOV60b ------------------------------------------------------------
NOV60c ------------------------------------------------------------
NOV60d DGRTFVEYLNAVTESEKALQSNHFELSLRTEATQGLVLWSGKATERADYVALAIVDGHLQ
NOV60e ------------------------------------------------------------
NOV60f ------------------------------------------------------------
NOV60g ------------------------------------------------------------
NOV60h ------------------------------------------------------------
NOV60i FDGRTFVEYLNAVTESEKALQSNHFELSLRTEATQGLVLWSGKATERADYVALAIVDGHL
NOV60a ------------------------------------------------------------
NOV60b ------------------------------------------------------------
NOV60c ------------------------------------------------------------
NOV60d LSYNLGSQPVVLRSTVPVNTNRWLRVVAHREQREGSLQVGNEAPVTGSSPLGATQLDTDG
NOV60e ------------------------------------------------------------
NOV60f ------------------------------------------------------------
NOV60g ------------------------------------------------------------
NOV60h ------------------------------------------------------------
NOV60i QLSYNLGSQPVVLRSTVPVNTNRWLRVVAHREQREGSLQVGNEAPVTGSSPLGATQLDTD
NOV60a ---------------------------------------------------------
NOV60b ---------------------------------------------------------
NOV60c ---------------------------------------------------------
NOV60d ALWLGGLPELPVGPALPKAYGTGFVGCLRDVVVGRHPLHLLEDAVTKPELRPCPTP-
NOV60e ---------------------------------------------------------
NOV60f ---------------------------------------------------------
NOV60g ---------------------------------------------------------
NOV60h ---------------------------------------------------------
NOV60i GALWLGGLPELPVGPALPKAYGTGFVGCLRDVVVGRHPLHLLEDAVTKPELRPCPTP
NOV60a (SEQ ID NO: 1174)
NOV60b (SEQ ID NO: 1176)
NOV60c (SEQ ID NO: 1178)
NOV60d (SEQ ID NO: 1180)
NOV60e (SEQ ID NO: 1182)
NOV60f (SEQ ID NO: 1184)
NOV60g (SEQ ID NO: 1186)
NOV60h (SEQ ID NO: 1188)
NOV60i (SEQ ID NO: 1190)

Further analysis of the NOV60a protein yielded the following properties shown in Table 60C.

TABLE 60C
Protein Sequence Properties NOV60a

| | |
|---|---|
| SignalP analysis: | Cleavage site between residues 34 and 35 |
| PSORT II analysis: | |

| PSG: | a new signal peptide prediction method | |
|---|---|---|
| | N-region: | length 5; pos. chg 2; neg. chg 0 |
| | H-region: | length 9; peak value 3.79 |
| | PSG score: | −0.61 |

| | |
|---|---|
| GvH: | von Heijne's method for signal seq. recognition |
| | GvH score (threshold: −2.1): 0.49 |
| | possible cleavage site: between 33 and 34 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 1 | | |
| | Number of TMS(s) for threshold 0.5: 1 | | |
| | INTEGRAL | Likelihood = | Transmembrane 17-33 |
| | | −4.35 | |

| | |
|---|---|
| PERIPHERAL | Likelihood = 0.53 (at 609) |

| | |
|---|---|
| | ALOM score: −4.35 (number of TMSs: 1) |
| MTOP: | Prediction of membrane topology (Hartmann et al.) |
| | Center position for calculation: 24 |
| | Charge difference: −6.5 C(−2.0)-N(4.5) |
| | N >= C: N-terminal side will be inside |

>>> membrane topology: type 2 (cytoplasmic tail 1 to 17)

| MITDISC: | discrimination of mitochondrial targeting seg | | | |
|---|---|---|---|---|
| | R content: | 3 | Hyd Moment(75): | 2.17 |
| | Hyd Moment(95): | 10.07 | G content: | 6 |
| | D/E content: | 1 | S/T content: | 1 |

| | |
|---|---|
| | Score: −4.90 |
| Gavel: | prediction of cleavage sites for mitochondrial preseq |
| | R-2 motif at 25 GRP\|LL |

NUCDISC: discrimination of nuclear localization signals

| | |
|---|---|
| | pat4: none |
| | pat7: PRTRRPE (5) at 339 |
| | pat7: PKSRKVP (5) at 1755 |
| | bipartite: none |
| | content of basic residues: 9.5% |
| | NLS Score: 0.39 |
| KDEL: | ER retention motif in the C-terminus: none |

ER Membrane Retention Signals:

XXRR-like motif in the N-terminus: RHGR

none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: nuclear |
| | Reliability: 76.7 |
| COIL: | Lupas's algorithm to detect coiled-coil regions |
| | total: 0 residues |

Final Results (k = 9/23):

```
34.8%: mitochondrial
34.8%: nuclear
13.0%: cytoplasmic
  4.3%: extracellular, including cell wall
  4.3%: vacuolar
  4.3%: Golgi
  4.3%: peroxisomal
```

>> prediction for CG94946-01 is mit (k = 23)

A search of the NOV60a protein against the Geneseq database, a proprietary database that contains sequences published in patents and patent publication, yielded several homologous proteins shown in Table 60D.

TABLE 60D
Geneseq Results for NOV60a

| | | NOV60a | Identities/ | | |
|---|---|---|---|---|---|
| | Protein/Organism/Length | Residues/ | Similarities for | | |
| Geneseq | [Patent #, | Match | the Matched | | Expect |
| Identifier | Date] | Residues | Region | | Value |
| ABU52400 | Human GPCR related protein | 160 . . . 2053 | 1841/1894 | (97%) | 0.0 |
| | NOV40a - Homo sapiens, 1931 aa. | 51 . . . 1931 | 1853/1894 | (97%) | |
| | [WO200279398-A2, 10-OCT-2002] | | | | |
| ABP43859 | Human mRNA precursor - Homo | 137 . . . 1669 | 1530/1533 | (99%) | 0.0 |
| | sapiens, 1741 aa. [WO200231111- | 1 . . . 1533 | 1530/1533 | (99%) | |
| | A2, 18-APR-2002] | | | | |
| AAW26609 | Human agrin - Homo sapiens, 492 | 1591 . . . 2053 | 460/471 | (97%) | 0.0 |
| | aa. [WO9721811-A2, 19-JUN-1997] | 22 . . . 492 | 461/471 | (97%) | |
| AAB93754 | Human protein sequence SEQ ID | 583 . . . 968 | 381/386 | (98%) | 0.0 |
| | NO: 13424 - Homo sapiens, 413 aa. | 1 . . . 386 | 384/386 | (98%) | |
| | [EP1074617-A2, 07-FEB-2001] | | | | |
| AAY73993 | Human prostate tumor EST fragment | 1634 . . . 2053 | 414/420 | (98%) | 0.0 |
| | derived protein #180 - Homo sapiens, | 1 . . . 416 | 414/420 | (98%) | |
| | 416 aa. [DE19820190-A1, 04-NOV-1999] | | | | |

In a BLAST search of public sequence databases, the NOV60a protein was found to have homology to the proteins shown in the BLASTP data in Table 60E.

TABLE 60E
Public BLASTP Results for NOV60a

| | | NOV60a | | | |
|---|---|---|---|---|---|
| Protein | | Residues/ | Identities/ | | |
| Accession | | Match | Similarities for the | | Expect |
| Number | Protein/Organism/Length | Residues | Matched Portion | | Value |
| O00468 | AGRIN precursor - Homo sapiens | 24 . . . 2053 | 2022/2030 | (99%) | 0.0 |
| | (Human), 2026 aa (fragment). | 1 . . . 2026 | 2022/2030 | (99%) | |
| P25304 | Agrin precursor - Rattus | 160 . . . 2053 | 1558/1914 | (81%) | 0.0 |
| | norvegicus (Rat), 1959 aa. | 51 . . . 1959 | 1663/1914 | (86%) | |
| P31696 | Agrin precursor - Gallus gallus | 128 . . . 2050 | 1234/1970 | (62%) | 0.0 |
| | (Chicken), 1955 aa. | 1 . . . 1952 | 1479/1970 | (74%) | |
| Q90404 | Agrin - Discopyge ommata | 716 . . . 2051 | 733/1353 | (54%) | 0.0 |
| | (Electric ray), 1328 aa (fragment). | 1 . . . 1325 | 932/1353 | (68%) | |
| Q96IC1 | Hypothetical protein - Homo | 1562 . . . 2053 | 486/492 | (98%) | 0.0 |
| | sapiens (Human), 488 aa | 1 . . . 488 | 486/492 | (98%) | |
| | (fragment). | | | | |

PFam analysis predicts that the NOV60a protein contains the domains shown in the Table 60F.

TABLE 60F
Domain Analysis of NOV60a

| | | Identities/ | | |
|---|---|---|---|---|
| Pfam | NOV60a | Similarities for | | Expect |
| Domain | Match Region | the Matched Region | | Value |
| NtA | 34 . . . 161 | 125/135 | (93%) | 1.1e−111 |
| | | 128/135 | (95%) | |
| kazal | 201 . . . 246 | 25/61 | (41%) | 7.2e−18 |
| | | 36/61 | (59%) | |
| kazal | 276 . . . 321 | 21/62 | (34%) | 5.1e−13 |
| | | 33/62 | (53%) | |
| kazal | 346 . . . 393 | 18/61 | (30%) | 7.9e−12 |
| | | 33/61 | (54%) | |
| kazal | 420 . . . 465 | 21/61 | (34%) | 4.1e−16 |
| | | 38/61 | (62%) | |
| kazal | 494 . . . 538 | 24/61 | (39%) | 3.6e−19 |
| | | 38/61 | (62%) | |
| kazal | 559 . . . 603 | 19/61 | (31%) | 1.4e−18 |
| | | 38/61 | (62%) | |
| kazal | 624 . . . 668 | 26/62 | (42%) | 1.5e−17 |
| | | 37/62 | (60%) | |
| kazal | 709 . . . 754 | 24/62 | (39%) | 1.2e−16 |
| | | 40/62 | (65%) | |
| laminin_EGF | 797 . . . 848 | 28/61 | (46%) | 1.1e−20 |
| | | 46/61 | (75%) | |
| laminin_EGF | 851 . . . 895 | 21/59 | (36%) | 3.6e−11 |
| | | 37/59 | (63%) | |
| kazal | 927 . . . 973 | 25/62 | (40%) | 5.2e−18 |
| | | 41/62 | (66%) | |
| SEA | 1134 . . . 1256 | 38/132 | (29%) | 4.6e−37 |
| | | 111/132 | (84%) | |
| EGF | 1337 . . . 1370 | 16/47 | (34%) | 0.00055 |
| | | 24/47 | (51%) | |
| laminin_G | 1404 . . . 1535 | 71/155 | (46%) | 7.4e−55 |
| | | 120/155 | (77%) | |
| EGF | 1557 . . . 1589 | 16/47 | (34%) | 5.2e−06 |
| | | 27/47 | (57%) | |
| EGF | 1596 . . . 1628 | 16/47 | (34%) | 0.00021 |
| | | 25/47 | (53%) | |
| laminin_G | 1672 . . . 1807 | 70/155 | (45%) | 7e−52 |
| | | 123/155 | (79%) | |
| EGF | 1826 . . . 1860 | 14/47 | (30%) | 4.3e−07 |
| | | 25/47 | (53%) | |
| laminin_G | 1905 . . . 2036 | 59/155 | (38%) | 2e−50 |
| | | 125/155 | (81%) | |

### Example 61

The NOV61 clone was analyzed, and the nucleotide and encoded polypeptide sequences are shown in Table 61A.

TABLE 61A
NOV61 Sequence Analysis

| | | |
|---|---|---|
| NOV61a, CG96384-01 | SEQ ID NO: 1191 | 789 bp |
| DNA Sequence | ORF Start: ATG at 1 | ORF Stop: TAA at 787 |

ATGCCTTGGGTATGCCGCCCCACTGGCTGGACAAAGCGGCGCGTGGATGTGCCTGTGGGGCCTTGCCC
TGGGCATCGCTGCTGCTGCCAGCGCCCTTCACCCATGCTTCAGCATGGTGCCCATACGCACTTCCTGC
AGGAGTCTGCTGGATACCTGCAGCTGGAGCACAGGAGAGATTTCAGCTCTTCTGGGAGTAGGAAGCTC
TCCTTTGACACTCGTTCCTTAGTGTGCTTTCTGGAAGACCATGGGTTTGCTACTCAGCAAGCAGAAAT
CATTGTGTCTGCATTGGTCCAGGTACTGGAGGCCAACGTGGACATCGTCTACAAAGATATGGCCACCA
AGATGAAGCAGGAGATCGCTCTTCAGCAAATAATGTCTCAGACTGTGAATGTGAAAAACGATATGATT
ACTTTGGAGAAGAGTGAATTTTCAGCCCTCAGAGCAGATCGTGAGAAAATAAAACTCAAACTACATCA
GTTAAAACAAGTAATGGATGAAGTGATTAAAGTCCGAACAGATACTAAATTAGACTTCAACCTAGAAA
AGAGCAGAGTAAAAGAATTGTATTCGTTGAATGAAAGGAAGCTGCTGGAATTGAGAACAGAAATAGTG
ACATTGCATGCCCAGCAAGATTGGGCCGTCACCCAGAGAGATAGGAAGATAGAAACTGAGGATGCTGG
CCCCAAAACCATGCTTGAGTCATACAAGCTTGATAATATTAAATATTTAGCAGGGTCTATATTTACGT
GCCTAACAGTAGCTCTGGGATTTTATCACCTGTGGATCTAA

| | | | |
|---|---|---|---|
| NOV61a, CG96384-01 | | | |
| Protein Sequence | SEQ ID NO: 1192 | 262 aa | MW at 30251.7kD |

MPWVCRPTGWTKRRVDVPVGPCPGHRCCCQRPSPMLQHGAHTHFLQESAGYLQLEHRRDFSSSGSRKL
SFDTRSLVCFLEDHGFATQQAEIIVSALVQVLEANVDIVYKDMATKMKQEIALQQIMSQTVNVKNDMI
TLEKSEFSALRAEREKIKLKLHQLKQVMDEVIKVRTDTKLDFNLEKSRVKELYSLNERKLLELRTEIV
TLHAQQDWAVTQRDRKIETEDAGPKTMLESYKLDNIKYLAGSIFTCLTVALGFYHLWI

| | | |
|---|---|---|
| NOV61b, 277580745 | SEQ ID NO: 1193 | 1808 bp |
| DNA Sequence | ORF Start: at 2 | ORF Stop: end of sequence |

CACCGGATCCACCATGCCTTGGGTATGCCGCCCCACTGGCTGGACAAAGCGGCGCGTGGATGTGCCTG
TGGGGCCTTGCCCTGGGCATCGCTGCTGCTGCCAGCGCCCTTCACCCATGCTTCAGCATGGTGCCTAT
ACTCACTTCCTGCAGGAGTCTGCTGGATACCTGCAGCTGGAGCACAGGAGAGATTTCAGCTCTTCTGG
GAGTAGGAAGCTCTCCTTTGACACTCGTTCCTTAGTGTGCTTTCTGGAAGACCATGGGTTTGCTACTC
AGCAAGCAGAAATCATTGTGTCTGCATTGGTCCAGGTACTGGAGGCCAACGTGGACATCGTCTACAAA
GATATGGCCACCAAGATGAAGCAGGAGATCGCTCTTCAGCAAATAATGTCTCAGACTGTGAATGTGAA
AAACGATATGATTACTTTGGAGAAGAGTGAATTTTCAGCCCTCAGAGCAGAACGTGAGAAAATAAAAC
TCAAACTACATCAGTTAAAACAAGTAATGGATGAAGTGATTAAAGTCCGAACAGATACTAAATTAGAC
TTCAACCTAGAAAAGAGCAGAGTAAAAGAATTGTATTCGTTGAATGAAAGGAAGCTGCTGGAATTGAG
AACAGAAATAGTGACATTGCATGCCCAGCAAGATTGGGCCGTCACCCAGAGAGATAGGAAGATAGAAA
CTGAGGATGCTGGCCCCAAAACCATGCTTGAGTCATACAAGCTTGATAATATTAAATATTTAGCAGGG
TCTATATTTACGTGCCTAACAGTAGCTCTGGGATTTTATCACCTGTGGATCCTCGAGGGC

| | | | |
|---|---|---|---|
| NOV61b, 277580745 | | | |
| Protein Sequence | SEQ ID NO: 1194 | 269 aa | MW at 30923.4kD |

TGSTMPWVCRPTGWTKRRVDVPVGPCPGHRCCCQRPSPMLQHGAYTHFLQESAGYLQLEHRRDFSSSG
SRKLSFDTRSLVCFLEDHGFATQQAEIIVSALVQVLEANVDIVYKDMATKMKQEIALQQIMSQTVNVK
NDMITLEKSEFSALRAEREKIKLKLHQLKQVMDEVIKVRTDTKLDFNLEKSRVKELYSLNERKLLELR
TEIVTLHAQQDWAVTQRDRKIETEDAGPKTMLESYKLDNIKYLAGSIFTCLTVALGFYHLWILEG

| | | |
|---|---|---|
| NOV61c, CG96384-02 | SEQ ID NO: 1195 | 789 bp |
| DNA Sequence | ORF Start: ATG at 1 | ORF Stop: TAA at 787 |

ATGCCTTGGGTATGCCGCCCCACTGGCTGGACAAAGCGGCGCGTGGATGTGCCTGTGGGGCCTTGCCC
TGGGCATCGCTGCTGCTGCCAGCGCCCTTCACCCATGCTTCAGCATGGTGCCCATACGCACTTCCTGC
AGGAGTCTGCTGGATACCTGCAGCTGGAGCACAGGAGAGATTTCAGCTCTTCTGGGAGTAGGAAGCTC
TCCTTTGACACTCGTTCCTTAGTGTGCTTTCTGGAAGACCATGGGTTTGCTACTCAGCAAGCAGAAAT
CATTGTGTCTGCATTGGTCCAGGTACTGGAGGCCAACGTGGACATCGTCTACAAAGATATGGCCACCA
AGATGAAGCAGGAGATCGCTCTTCAGCAAATAATGTCTCAGACTGTGAATGTGAAAAACGATATGATT
ACTTTGGAGAAGAGTGAATTTTCAGCCCTCAGAGCAGAACGTGAGAAAATAAAACTCAAACTACATCA
GTTAAAACAAGTAATGGATGAAGTGATTAAAGTCCGAACAGATACTAAATTAGACTTCAACCTAGAAA
AGAGCAGAGTAAAAGAATTGTATTCGTTGAATGAAAGGAAGCTGCTGGAATTGAGAACAGAAATAGTG
ACATTGCATGCCCAGCAAGATTGGGCCGTCACCCAGAGAGATAGGAAGATAGAAACTGAGGATGCTGG
CCCCAAAACCATGCTTGAGTCATACAAGCTTGATAATATTAAATATTTAGCAGGGTCTATATTTACGT
GCCTAACAGTAGCTCTGGGATTTTATCACCTGTGGATCTAA

| | | | |
|---|---|---|---|
| NOV61c, CG96384-02 | | | |
| Protein Sequence | SEQ ID NO: 1196 | 1262 aa | MW at 30251.7kD |

MPWVCRPTGWTKRRVDVPVGPCPGHRCCCQRPSPMLQHGAHTHFLQESAGYLQLEHRRDFSSSGSRKL
SFDTRSLVCFLEDHGFATQQAEIIVSALVQVLEANVDIVYKDMATKMKQEIALQQIMSQTVNVKNDMI
TLEKSEFSALRAEREKIKLKLHQLKQVMDEVIKVRTDTKLDFNLEKSRVKELYSLNERKLLELRTEIV
TLHAQQDWAVTQRDRKIETEDAGPKTMLESYKLDNIKYLAGSIFTCLTVALGFYHLWI

| | | |
|---|---|---|
| NOV61d, CG96384-03 | SEQ ID NO: 1197 | 1285 bp |
| DNA Sequence | ORF Start: at 31 | ORF Stop: at 280 |

GGATCCACCATGCCTTGGGTATGCCGCCCCACTGGCTGGACAAAGCGGCGCGTGGATGTGCCTGTGGG
GCCTTGCCCTGGGCATCGCTGCTGCTGCCAGCGCCCTTCACCCATGCTTCAGCATGGTGCCTATACTC
ACTTCCTGCAGGAGTCTGCTGGATACCTGCAGCTGGAGCACAGGAGAGATTTCAGCTCTTCTGGGAGT
AGGAAGCTCTCCTTTGACACTCGTTCCTTAGTGTGCTTTCTGGAAGACCATGGGTTTGCTACTCAGCA
AGCAGAACTCGAG

| | | | |
|---|---|---|---|
| NOV61d, CG96384-03 | | | |
| Protein Sequence | SEQ ID NO: 1198 | 183 aa | MW at 9410.5kD |

TGWTKRRVDVPVGPCPGHRCCCQRPSPMLQHGAYTHFLQESAGYLQLEHRPDFSSSGSRKLSFDTRSL
VCFLEDHGFATQQAE

| | | |
|---|---|---|
| NOV61e, CG96384-04 | SEQ ID NO: 1199 | 1801 bp |
| DNA Sequence | ORF Start: at 31 | ORF Stop: at 772 |

GGATCCACCATGCCTTGGGTATGCCGCCCCACTGGCTGGACAAAGCGGCGCGTGGATGTGCCTGTGGG
GCCTTGCCCTGGGCATCGCTGCTGCTGCCAGCGCCCTTCACCCATGCTTCAGCATGGTGCCTATACTC
ACTTCCTGCAGGAGTCTGCTGGATACCTGCAGCTGGAGCACAGGAGAGATTTCAGCTCTTCTGGGAGT
AGGAAGCTCTCCTTTGACACTCGTTCCTTAGTGTGCTTTCTGGAAGACCATGGGTTTGCTACTCAGCA
AGCAGAAATCATTGTGTCTGCATTGGTCCAGGTACTGGAGGCCAACGTGGACATCGTCTACAAAGATA
TGGCCACCAAGATGAAGCAGGAGATCGCTCTTCAGCAAATAATGTCTCAGACTGTGAATGTGAAAAAC
GATATGATTACTTTGGAGAAGAGTGAATTTTCAGCCCTCAGAGCAGAACGTGAGAAAATAAAACTCAA
ACTACATCAGTTAAACAAGTAATGGATGAAGTGATTTAAAGTCCGAACAGATACTAAATTAGACTTCA
ACCTAGAAAAGAGCAGAGTAAAAGAATTGTATTCGTTGAATGAAAGGAAGCTGCTGGAATTGAGAACA
GAAATAGTGACATTGCATGCCCAGCAAGATTGGGCCGTCACCCAGAGAGATAGGAAGATAGAAACTGA
GGATGCTGGCCCCAAAACCATGCTTGAGTCATACAAGCTTGATAATATTAAATATTTAGCAGGGTCTA
TATTTACGTGCCTAACAGTAGCTCTGGGATTTTATCACCTGTGGATCCTCGAG

| | | | |
|---|---|---|---|
| NOV61e, CG96384-04 | | | |
| Protein Sequence | SEQ ID NO: 1200 | 247 aa | MW at 28377.4kD |

TGWTKRRVDVPVGPCPGHRCCCQRPSPMLQHGAYTHFLQESAGYLQLEHRRDFSSSGSRKLSFDTRSL
VCFLEDHGFATQQAEIIVSALVQVLEANVDIVYKDMATKMKQEIALQQIMSQTVNVKNDMITLEKSEF
SALRAEREKIKLKLHQLKQVMDEVIKVRTDTKLDFNLEKSRVKELYSLNERKLLELRTEIVTLHAQQD
WAVTQRDRKIETEDAGPKTMLESYKLDNIKYLAGSIFTCLTVA

A ClustalW comparison of the above protein sequences yields the following sequence alignment shown in Table 61B.

TABLE 61B
Comparison of the NOV61 protein sequences.

| | |
|---|---|
| NOV61a | ----MPWVCRPTGWTKRRVDVPVGPCPGHRCCCQRPSPMLQHGAHTHFLQESAGYLQLEH |
| NOV61b | TGSTMPWVCRPTGWTKRRVDVPVGPCPGHRCCCQRPSPMLQHGAYTHFLQESAGYLQLEH |
| NOV61c | ----MPWVCRPTGWTKRRVDVPVGPCPGHRCCCQRPSPMLQHGAHTHFLQESAGYLQLEH |
| NOV61d | -----------TGWTKRRVDVPVGPCPGHRCCCQRPSPMLQHGAYTHFLQESAGYLQLEH |
| NOV61e | -----------TGWTKRRVDVPVGPCPGHRCCCQRPSPMLQHGAYTHFLQESAGYLQLEH |
| N0V61a | RRDFSSSGSRKLSFDTRSLVCFLEDHGFATQQAEIIVSALVQVLEANVDIVYKDMATKMK |
| N0V61b | RRDFSSSGSRKLSFDTRSLVCFLEDHGFATQQAEIIVSALVQVLEANVDIVYKDMATKMK |
| N0V61c | RRDFSSSGSRKLSFDTRSLVCFLEDHGFATQQAEIIVSALVQVLEANVDIVYKDMATKMK |
| NOV61d | RRDFSSSGSRKLSFDTRSLVCFLEDHGFATQQAE-------------------------- |
| N0V61e | RRDFSSSGSRKLSFDTRSLVCFLEDHGFATQQAEIIVSALVQVLEANVDIVYKDMATKMK |
| NOV61a | QEIALQQIMSQTVNVKNDMITLEKSEFSALRAEREKIKLKLHQLKQVMDEVIKVRTDTKL |
| NOV61b | QEIALQQIMSQTVNVKNDMITLEKSEFSALRAEREKIKLKLHQLKQVMDEVIKVRTDTKL |
| NOV61c | QEIALQQIMSQTVNVKNDMITLEKSEFSALRAEREKIKLKLHQLKQVMDEVIKVRTDTKL |
| NOV61d | ------------------------------------------------------------ |
| NOV61e | QEIALQQIMSQTVNVKNDMITLEKSEFSALRAEREKIKLKLHQLKQVMDEVIKVRTDTKL |
| NOV61a | DFNLEKSRVKELYSLNERKLLELRTEIVTLHAQQDWAVTQRDRKIETEDAGPKTMLESYK |
| NOV61b | DFNLEKSRVKELYSLNERKLLELRTEIVTLHAQQDWAVTQRDRKIETEDAGPKTMLESYK |
| NOV61c | DFNLEKSRVKELYSLNERKLLELRTEIVTLHAQQDWAVTQRDRKIETEDAGPKTMLESYK |
| NOV61d | ------------------------------------------------------------ |
| NOV61e | DFNLEKSRVKELYSLNERKLLELRTEIVTLHAQQDWAVTQRDRKIETEDAGPKTMLESYK |
| NOV61a | LDNIKYLAGSIFTCLTVALGFYHLWI--- |
| NOV61b | LDNIKYLAGSIFTCLTVALGFYHLWILEG |
| NOV61c | LDNIKYLAGSTFTCLTVALGFYHLWI--- |
| NOV61d | ----------------------------- |
| NOV61e | LDNIKYLAGSIFTCLTVA----------- |
| NOV61a | (SEQ IDNO: 1192) |
| NOV61b | (SEQ IDNO: 1194) |
| NOV61c | (SEQ IDNO: 1196) |
| NOV61d | (SEQ IDNO: 1198) |
| NOV61e | (SEQ IDNO: 1200) |

Further analysis of the NOV61a protein yielded the following properties shown in Table 61C.

TABLE 61C
Protein Sequence Properties NOV61a

| | |
|---|---|
| SignalP analysis: | No Known Signal Sequence Predicted |
| PSORT II analysis: | |

| PSG: | a new signal peptide prediction method | |
|---|---|---|
| | N-region: | length 6; pos. chg 1; neg. chg 0 |
| | H-region: | length 5; peak value −14.81 |
| | PSG score: | −19.21 |

| | |
|---|---|
| GvH: | von Heijne's method for signal seq. recognition |
| | GvH score (threshold: −2.1): −11.24 |
| | possible cleavage site: between 29 and 30 |

>>> Seems to have no N-terminal signal peptide

| ALOM: | Klein et al's method for TM region allocation | | |
|---|---|---|---|
| | Init position for calculation: 1 | | |
| | Tentative number of TMS(s) for the threshold 0.5: 2 | | |
| | Number of TMS(s) for threshold 0.5: 1 | | |
| | INTEGRAL | Likelihood = | Transmembrane 91-107 |
| | | −2.13 | |

| | |
|---|---|
| PERIPHERAL | Likelihood = 11.30 (at 68) |

| | |
|---|---|
| | ALOM score: −2.13 (number of TMSs: 1) |
| MTOP: | Prediction of membrane topology (Hartmann et al.) |
| | Center position for calculation: 98 |
| | Charge difference: 1.5 C(−1.0)-N(−2.5) |
| | C > N: C-terminal side will be inside |

>>> membrane topology: type 1b (cytoplasmic tail 91 to 262)

| MITDISC: | discrimination of mitochondrial targeting seq | | | |
|---|---|---|---|---|
| | R content: | 3 | Hyd Moment(75): | 7.07 |
| | Hyd Moment(95): | 9.21 | G content: | 1 |
| | D/E content: | 1 | S/T content: | 2 |

| | |
|---|---|
| | Score: −1.63 |
| Gavel: | prediction of cleavage sites for mitochondrial preseq |
| | R-10 motif at 68 RRD FS |

NUCDISC: discrimination of nuclear localization signals

| | |
|---|---|
| | pat4: none |
| | pat7: none |
| | bipartite: none |
| | content of basic residues: 14.1% |
| | NLS Score: −0.47 |
| KDEL: | ER retention motif in the C-terminus: none |

ER Membrane Retention Signals: none
SKL: peroxisomal targeting signal in the C-terminus: none
PTS2: 2nd peroxisomal targeting signal: found
KIKLKLHQL at 152
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: found

LL at 196

checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 76.7 |
| COIL: | Lupas's algorithm to detect coiled-coil regions |
| 138 E | 0.87 |
| 139 K | 0.87 |
| 140 S | 0.87 |
| 141 E | 0.87 |
| 142 F | 0.87 |
| 143 S | 0.87 |
| 144 A | 0.87 |
| 145 L | 0.87 |
| 146 R | 0.87 |
| 147 A | 0.87 |
| 148 E | 0.87 |
| 149 R | 0.87 |
| 150 E | 0.87 |
| 151 K | 0.87 |
| 152 I | 0.87 |
| 153 K | 0.87 |
| 154 L | 0.87 |
| 155 K | 0.87 |
| 156 L | 0.87 |
| 157 H | 0.87 |
| 158 Q | 0.87 |
| 159 L | 0.87 |
| 160 K | 0.87 |
| 161 Q | 0.87 |
| 162 V | 0.87 |
| 163 M | 0.87 |
| 164 D | 0.87 |
| 165 E | 0.87 |
| 166 V | 0.86 |
| 167 I | 0.79 |
| 168 K | 0.79 |
| 169 V | 0.59 |
| 170 R | 0.59 |
| 171 T | 0.59 |
| 172 D | 0.59 |
| | total: 35 residues |

Final Results (k = 9/23):

```
34.8%: nuclear
21.7%: mitochondrial
21.7%: cytoplasmic
  8.7%: vesicles of secretory system
  4.3%: vacuolar
  4.3%: peroxisomal
  4.3%: endoplasmic reticulum
```

>> prediction for CG96384-01 is nuc (k = 23)

A search of the NOV61a protein against the Geneseq database, a proprietary database that contains sequences published in patents and patent publication, yielded several homologous proteins shown in Table 61D.

TABLE 61D
Geneseq Results for NOV61a

| | | NOV61a | Identities/ | | |
|---|---|---|---|---|---|
| | Protein/Organism/Length | Residues/ | Similarities for | | |
| Geneseq | [Patent #, | Match | the Matched | | Expect |
| Identifier | Date] | Residues | Region | | Value |
| AAM40693 | Human polypeptide SEQ ID NO 5624 - | 45 . . . 262 | 185/220 | (84%) | 2e−95 |
| | Homo sapiens, 246 aa. | 27 . . . 246 | 197/220 | (89%) | |
| | [WO200153312-A1, 26-JUL-2001] | | | | |
| AAM38907 | Human polypeptide SEQ ID NO 2052 - | 104 . . . 262 | 138/160 | (86%) | 3e−70 |
| | Homo sapiens, 160 aa. | 1 . . . 160 | 145/160 | (90%) | |
| | [WO200153312-A1, 26-JUL-2001] | | | | |
| AAG73708 | Human colon cancer antigen protein | 142 . . . 262 | 110/122 | (90%) | 9e−54 |
| | SEQ ID NO: 4472 - Homo sapiens, 129 | 8 . . . 129 | 114/122 | (93%) | |
| | aa. [WO200122920-A2, 05-APR-2001] | | | | |
| ABU53059 | Human testes-derived protein from | 59 . . . 261 | 96/204 | (47%) | 3e−48 |
| | DKFZphtes3_19f19 - Homo sapiens, | 50 . . . 253 | 138/204 | (67%) | |
| | 254 aa. [WO200112659-A2, 22-FEB-2001] | | | | |
| ABB89935 | Human polypeptide SEQ ID NO 2311 - | 59 . . . 261 | 96/204 | (47%) | 3e−48 |
| | Homo sapiens, 245 aa. | 41 . . . 244 | 138/204 | (67%) | |
| | [WO200190304-A2, 29-NOV-2001] | | | | |

In a BLAST search of public sequence databases, the NOV61a protein was found to have homology to the proteins shown in the BLASTP data in Table 61E.

TABLE 61E
Public BLASTP Results for NOV61a

| | | NOV61a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| Q96JS7 | Similar to RIKEN cDNA 6230416A05 | 45 . . . 262 | 186/220 | (84%) | 1e−96 |
| | gene - Homo sapiens (Human), 321 | 102 . . . 321 | 198/220 | (89%) | |
| | aa (fragment). | | | | |
| Q96AQ8 | Similar to RIKEN cDNA 6230416A05 | 45 . . . 262 | 185/220 | (84%) | 6e−95 |
| | gene - Homo sapiens (Human), 359 | 140 . . . 359 | 197/220 | (89%) | |
| | aa. | | | | |
| Q9NUI2 | DJ500L14.1 (Novel protein) - Homo | 45 . . . 262 | 185/220 | (84%) | 6e−95 |
| | sapiens (Human), 220 aa (fragment). | 1 . . . 220 | 197/220 | (89%) | |
| Q9CXD6 | 6230416A05Rik protein - Mus | 45 . . . 262 | 163/220 | (74%) | 3e−83 |
| | musculus (Mouse), 340 aa. | 121 . . . 340 | 188/220 | (85%) | |
| Q9GZT6 | MDS011 (MDS025) (Hypothetical | 59 . . . 261 | 96/204 | (47%) | 9e−48 |
| | protein) - Homo sapiens (Human), | 50 . . . 253 | 138/204 | (67%) | |
| | 254 aa. | | | | |

PFam analysis predicts that the NOV61a protein contains the domains shown in the Table 61F.

TABLE 61F
Domain Analysis of NOV61a

| | | | |
|---|---|---|---|
| Pfam | NOV61a | Identities/ | Expect |
| Domain | Match Region | Similarities for | Value |
| | | the Matched Region | |

### Example 62

The NOV62 clone was analyzed, and the nucleotide and encoded polypeptide sequences are shown in Table 62A.

TABLE 62A
NOV62 Sequence Analysis

| | | |
|---|---|---|
| NOV62a, CG98011-01 | SEQ ID NO: 1201 | 12631 bp |
| DNA Sequence | ORF Start: ATG at 1 | ORF Stop: TAG at 2629 |

ATGATTTACATACAAGTAATTTTTCAAGTAATGACCATTGAAAAAATGTTTTCTTTTTATTTTTTAGA
TTATTTCTCTTTATTCAGAAGCATACAGTTGTTTGCTGATTGCAAGAAGATGTTTCTGTGGCTGTTTC
TGATTTTGTCAGCCCTGATTTCTTCGACAAATGCAGATTCTGACATATCGGTGGAAATTTGCAATGTG
TGTTCCTGCGTGTCAGTTGAGAATGTGCTCTATGTCAACTGTGAGAAGGTTTCAGTCTACAGACCAAA
CAGCTAGAAACCACCTTGGTCTAATTTTTATCACCTCAATTTCCAAAATAATTTTTTAAATATTCTGT
ATCCAAATACATTCTTGAATTTTTCACATGCAGTCTCCCTGCATCTGGGGAATAATAAACTGCAGAAC
ATTGAGGGAGGAGCCTTTCTTGGGCTCAGTGCATTAAAGCAGTTGCACTTGAACAACAATGAATTAAA
GATTCTCCGAGCTGACACTTTCCTTGGCATAGAGAACTTGGAGTATCTCCAGGCTGACTACAATTTAA
TCAAGTATATTGAACTGAGGAGCCTTCAATAAGCTCCACAAACTGAAGTTCTCATTCTTTTTGACAAT
CTGATTTCATTCCTTCCTGATAATATTTTCCGATTCGCATCTTTGACCCATCTGGATATACGAGGGAA
CAGAATCCAGAAGCTCCCTTATATCGGGGTTCTGGAACACATTGGCCGTGTCGTTGAATTGCAACTGG
AACATTTACATAGGAGAAGCTATCTGTGAAACTCCCAGTGACTTATATGGAAGGCTTTTAAAAGAAAC
AGCTGGAAAATGGCTACACCACTCCCAATGGTCACACTACCCAAACATCTTTACACAGATTAGTAACT
AAACCACCAAAAACAACAAATCCTTCCAAGATCTCTGGAATCGTTGCAGGCAAAGCCCTCTCCAACCG
CAATCTCAGTCAGATTGTGTCTTACCAAACAAGGGTGCCTCCTCTAACACCTTGCCCGGCACCTTGCT
TCTGCAAAACACACCCTTCAGATTTGGGACTAAGTGTGAACTGCCAAGAGAAAAATATACAGTCTATG
TCTGAACTGATACCGAAACCTTTAAATGCGAAGAAGCTGCACGTCAATGGCAATAGCATCAAGGATGT
GGACGTATCAGACTTCACTGACTTTGAAGGACTGGATTTGCTTCATCTAGGCAGCAATCAAATTACAG
TGATTAAGGGAGACGTATTTCACAATCTCACTAATTTACGCAGGCTATATCTCAATGGCAATCAAATT
GAGAGACTCTATCCTGAAATATTTTCAGGTCTTCATAACCTGCAGTATCTGTATTTGGAATACAATTT
GATTAAGGAAATCTCAGCAGGCACCTTTGACTCCATGCCAAATTTGCAGTTACTGTACTTAAACAATA
ATCTCCTAAAGAGCCTGCCTGTTTACATCTTTTCCGGAGCACCCTTAGCTAGACTGAACCTGAGGAAC
AACAAATTCATGTACCTGCCTGTCAGTGGGGTCCTTGATCAGTTGCAATCTCTTACACAGATTGACTT
GGAGGGCAACCCATGGGACTGTACTTGTGACTTGGTGGCATTAAAGCTGTGGGTGGAGAAGTTGAGCG
ACGGGATTGTTGTGAAAGAACTGAAATGTGAGACGCCTGTTCAGTTTGCCAACATTGAACTGAAGTCC
CTCAAAAATGAAATCTTATGTCCCAAACTTTTAAATAAGCCGTCTGCACCATTCACAAGCCCTGCACC
TGCCATTACATTCACCACTCCTTTGGGTCCCATTCGAAGTCCTCCTGGTGGGCCAGTGCCTCTGTCTA
TTTTAATCTTAAGTATCTTAGTGGTCCTCATTTTAACGGTGTTTGTTGCTTTTTGCCTTCTTGTTTTT
GTCCTGCGACGCAACAAGAAACCCACAGTGAAGCACGAAGGCCTGGGGAATCCTGACTGTGGCTCCAT
GCAGCTGCAGCTAAGGAAGCATGACCACAAAACCAATAAAAAAGATGGACTGAGCACAGAAGCTTTCA
TTCCACAAACTATAGAACAGATGAGCAAGAGCCACACTTGTGGCTTGAAAGAGTCAGAAACTGGGTTC
ATGTTTTCAGATCCTCCAGGACAGAAAGTTGTTATGAGAAATGTGGCCGACAAGGAGAAAGATTTATT
ACATGTAGATACCAGGAAGAGACTGAGCACAATTGATGAGCTGGATGAATTATTCCCTAGCAGGGATT
CCAATGTGTTTATTCAGAATTTTCTTGAAAGCAAAAAGGAGTATAATAGCATAGGTGTCAGTGGCTTT
GAGATCCGCTATCCAGAAAAACAACCAGACAAAAAAAGTAAGAAGTCACTGATAGGTGGCAACCACAG
TAAAATTGTTGTGGAACAAAGGAAGAGTGAGTATTTTGAACTGAAGGCGAAACTGCAGAGTTCCCCTG
ACTACCTACAGGTCCTTGAGGAGCAAACAGCTTTGAACAAGATCTAG

| | | | |
|---|---|---|---|
| NOV62a, CG98011-01 | | | |
| Protein Sequence | SEQ ID NO: 1202 | 876 aa | MW at 99167.6kD |

MIYIQVIFQVMTIEKMFSFYFLDYFSLFRSIQLFADCKKMFLWLFLILSALISSTNADSDISVEICNV
CSCVSVENVLYVNCEKVSVYRPNQLKPPWSNFYHLNFQNNFLNILYPNTFLNFSHAVSLHLGNNKLQN
IEGGAFLGLSALKQLHLNNNELKILRADTFLGIENLEYLQADYNLIKYIERGAFNKLHKLKVLILNDN
LISFLPDNIFRFASLTHLDIRGNRIQKLPYIGVLEHIGRVVELQLEDNPWNCSCDLLPLKAWLENMPY
NIYIGEAICETPSDLYGRLLKETNKQELCPMGTGSDFDVRILPPSQLENGYTTPNGHTTQTSLHRLVT
KPPKTTNPSKISGIVAGKALSNRNLSQIVSYQTRVPPLTPCPAPCFCKTHPSDLGLSVNCQEKNIQSM
SELIPKPLNAKKLHVNGNSIKDVDVSDFTDFEGLDLLHLGSNQITVIKGDVFHNLTNLRPLYLNGNQI
ERLYPEIFSGLHNLQYLYLEYNLIKEISAGTFDSMPNLQLLYLNNNLLKSLPVYIFSGAPLAELNLRN
NKFMYLPVSGVLDQLQSLTQIDLEGNPWDCTCDLVALKLWVEKLSDGIVVKELKCETPVQFANIELKS
LKNEILCPKLLNKPSAPFTSPAPAITFTTPLGPIRSPPGGPVPLSILILSILVVLILTVFVAFCLLVF
VLRRNKKPTVKHEGLGNPDCGSMQLQLRKHDHKTAKKDGLSTEAFIPQTIEQMSKSHTCGLKESETGF
MFSDPPGQKVVMRNVADKEKDLLHVDTRKRLSTIDELDELFPSRDSNVFIQNFLESKKEYNSIGVSGF
EIRYPEKQPDKKSKKSLIGGNHSKIVVEQRKSEYFELKAXLQSSPDYLQVLEEQTALNKI

| | | |
|---|---|---|
| NOV62b, 192586956 | SEQ ID NO: 1203 | 1800 bp |
| DNA Sequence | ORF Start: at 1 | ORF Stop: end of sequence |

GGATCCGATTCTGACATATCGGTGGAAATTTGCAATGTGTGTTCCTGCGTGTCAGTTGAGAATGTGCT
CTATGTCAACTGTGAGAAGGTTTCAGTCTACAGACCAAATCAGCTGAAACCACCTTGGTCTAATTTTT
ATCACCTCAATTTCCAAAATAATTTTTTAAATATTCTGTATCCAAATACATTCTTGAATTTTTCACAT
GCAGTCTCCCTGCATCTGGGGAATAATAAACTGCAGAACATTGAGGGAGGAGCCTTTCTTGGGCTCAG
TACATTAAAGCAGTTGCACTTGAACAACAATGAATTAAAGATTCTCCGAGCTGACACTTTCCTTGGCA
TAGAGAACTTGGAGTATCTCCAGGCTGACTACAATTTAATCAAGTATATTGAACGAGGAGCCTTCAAT
AAGCTCCACAAACTGAAAGTTCTCATTCTTAATGACAATCTGATTTCATTCCTTCCTGATAATATTTT
CCGATTCGCATCTTTGACCCATCTGGATATACGAGGGAACAGAATCCAGAAGCTCCCTTATATCGGGG
TTCTGGAACACATTGGTCGTGTCGTTGAATTGCAACTGGAAGATAACCCTTGGAACTGTAGCTGTGAT
TTATTGCCCTTAAAAGCTTGGCTGGAGAACATGCCATATAACATTTACATAGGAGAAGCTATCTGTGA
AACTCCCAGTGACTTATATGGAAGGCTTTTAAAAGAAACCAACAAACAAGAGCTATGTCCCATGGGCA
CCGGCAGTGATTTTGACGTGCGCATCCTGCCTCCATCTCAGCTGGAAAATGGCTACACCACTCCCAAT
GGTCACACTACCCAAACATCTTTACACAGATTAGTAACTAAACCACCAAAAACAACAAATCCTTCCAA
GATCTCTGGAATCGTTGCAGGTAAAGCCCTCTCCAACCGCAATCTCAGTCAGATTGTGTCTTACCAAA
CAAGGGTGCCTCCTCTAACACCTTGCCCGGCACCTTGCTTCTGCAAAACACACCCTTCAGATTTGGGA
CTAAGTGTGAACTGCCAAGAGAAAAATATACAGTCTATGTCTGAACTGATACCGAAACCTTTAAATGC
GAAGAAGCTGCACGTCAATGGCAATAGCATCAAGGATGTGGACGTATCAGACTTCACTGACTTTGAAG
GACTGGATTTGCTTCATTTAGGCAGCAATCAAATTACAGTGATTAAGGGAGACGTATTTCACAATCTC
ACTAATTTACGCAGGCTATATCTCAATGGCAATCAAATTGAGAGACTCTATCCTGAAATATTTTCAGG
TCTTCATAACCTGCAGTATCTGTATTTGGAATACAATTTGATTAAGGAAATCTCAGCAGGCACCTTTG
ACTCCATGCCAAATTTGCAGTTACTGTACTTAAGCAATAATCTCCTAAAGAGCCTGCCTGTTTACATC
TTTTCCGGAGCACCCTTAGCTAGACTGAACCTGAGGAACAACATATTCATGTACCTGCCTGTCAGTGG
GGTCCTTGATCAGTTGCAATCTCTTACACAGATTGACTTGGAGGGCAGCCCATGGGACTATACTTGTG
ACTTGGTGGCATTAAAGCTGTGGGTGGAGAAGTTGAGCGACGGGATTGTTGTGAAAGAACTGAAATGT
TTTTCCGGAGCACCCTTAGCTAGACTGAACCTGAGGAACAACAAATTCATGTACCTGCCTGTCAGTGG
TTTAAATAAGCCGTCTGCACCATTCACAAGCCCTGCACCTGCCATTACATTCACCACTCCTTTGGGTC
CCATTCGAAGTCCTCCTGGTGGGCCACTCGAG

| | | | |
|---|---|---|---|
| NOV62b, 192586956 | | | |
| Protein Sequence | SEQ ID NO: 1204 | 1600 aa | MW at 67403.8kD |

GSDSDISVEICNVCSCVSVENVLYVNCEKVSVYRPNQLKPPWSNFYHLNFQNNFLNILYPNTFLNFSH
AVSLHLGNNKLQNIEGGAFLGLSTLKQLHLNNNELKILRADTFLGIENLEYLQADYNLIKYIERGAFN
KLHKLKVLILNDNLISFLPDNIFRFASLTHLDIRGNRIQKLPYIGVLEHIGRVVELQLEDNPWNCSCD
LLPLKAWLENMPYNIYIGEAICETPSDLYGRLLKETNKQELCPMGTGSDFDVRILPPSQLENGYTTPN
GHTTQTSLHRLVTKPPKTTNPSKISGIVAGKALSNRNLSQIVSYQTRVPPLTPCPAPCFCKTHPSDLG
LSVNCQEKNIQSMSELIPKPLNAKKLHVNGNSIKDVDVSDFTDFEGLDLLHLGSNQITVIKGDVFHNL
TNLRRLYLNGNQIERLYPEIFSGLHNLQYLYLEYNLIKEISAGTFDSMPNLQLLYLSNNLLKSLPVYI
FSGAPLARLNLRNNKFMYLPVSGVLDQLQSLTQIDLEGSPWDYTCDLVALKLWVEKLSDGIVVKELKC
IETPVQFTNIELKSLKEILCPKLLNKPSAPFTSPAPAITFTTPLGPIRSPPGGPLE

| | | |
|---|---|---|
| NOV62c, 191999007 | SEQ ID NO: 1205 | 1800 bp |
| DNA Sequence | ORF Start: at 1 | ORF Stop: end of sequence |

GGATCCGATTCTGACATATCGGTGGAAATTTGCAATGTGTGTTCCTGCGTGTCAGTTGAGAATGTGCT
CTATGTCAACTGTGAGAAGGTTTCAGTCTACAGACCAAATCAGCTGAAACCACCTTGGTCTAATTTTT
ATCACCTCAATTTCCAATAATTTTTTAATTTTATTCTGTATCCAAATACATTCTTGAATTTTTCACAT
GCAGTCTCCCTGCATCTGGGGAATAATAAACTGCAGAACATTGAGGGAGGAGCCTTTCTTGGGCTCAG
TGCATTAAAGCAGTTGCACTTGAACAACAATGAATTAAAGATTCTCCGAGCTGACACTTTCCCTGGCA
TAGAGAACTTGGAGTATCTCCAGGCTGACTACAATTTAATCAAGTATATTGAACGAGGAGCCTTCAAT
AAGCTCCACAAACTGAAAGTTCTCATTCTTAATGACAATCTGATTTCATTCCTTCCTGATAATATTTT
CCGATTCGCATCTTTGACCCATCTGGATATACGAGGGAACAGAATCCAGAAGCTCCCTTATATCGGGG
TTCTGGAACACATTGGCCGTGTCGTTGAATTGCAACTGGAAGATAACCCTTGGAACTGTAGCTGTGAT
TTATTGCCCTTAAAAGCTTGGCTGGAGAACATGCCATATAACATTTACATAGGAGAAGCTATCTGTGA
AACTCCCAGTGACTTATATGGAAGGCTTTTAAAAGAAACCAACAAACAAGAGCTATGTCCCATGGGCA
CCGGCAGTGATTTTGACGTGCGCATCCTGCCTCCATCTCAGCTGGAAAATGGCTACACCACTCCCAAT
GGTCACACTACCCAAACATCTTTACACAGATTAGTAACTAAACCACCAAAAACAACAAATCCTTCCAA
GATCTCTGGAATCGTTGCAGGCAAAGCCCTCTCCAACCGCAATCTCAGTCAGATTGTGTCTTACCAAA
CAAGGGTGCCTCCTCTAACACCTTGCCCGGCACCTTGCTTCTGCAAAACACACCCTTCAGATTTGGGA
CTAAGTGTGAACTGCCAAGAGAAAAATATACAGTCTATGTCTGAACTGATACCGAAACCTTTAAATGC
GAAGAAGCTGCACGTCAATGGCAATAGCATCAAGGATGTGGACGTATCAGACTTCACTGACTTTGAAG
GACTGGATTTGCTTCATTTAGGCAGCAATCAAATTACAGTGATTAAGGGAGACGTATTTCACAATCTC
ACTAATTTACGCAGGCTATATCTCAATGGCAATCAAATTGAGAGACTCTATCCTGAAATATTTTCAGG
TCTTCATAACCTGCAGTATCTGTATTTGGAATACAATTTGATTAAGGAAATCTCAGCAGGCACCTTTG
ACTCCATGCCAAATTTGCAGTTACTGTACTTAAACAATAATCTCCTAAAGAGCCTGCCTGTTTACATC
TTTTCCGGAGCACCCTTAGCTAGACTGAACCTGAGGAACAACAAATTCATGTACCTGCCTGTCAGTGG
GGTCCTTGATCAGTTGCAATCTCTTACACAGATTGACTTGGAGGGCAACCCATGGGACTGTACTTGTG
ACTTGGTGGCATTAAAGCTGTGGGTGGGGAAGTTGAGCGACGGGATTGTTGTGAAAGAACTGAAATGT
GAGACGCCTGTTCAGTTTGCCAACATTGAACTGAAGTCCCTCAAAAATGAAATCTTATGTCCCAAACT
TTTAAATAAGCCGTCTGCACCATTCACAAGCCCTGCACCTACCATTACATTCACCACTCCTTTGGGTC
CCATTCGAAGTCCTCCTGGTGGGCCACTCGAG

| | | | |
|---|---|---|---|
| NOV62c, 191999007 | | | |
| Protein Sequence | SEQ ID NO: 1206 | 600 aa | MW at 67279.7kD |

GSDSDISVEICNVCSCVSVENVLYVNCEKVSVYRPNQLKPPWSNFYHLNFQNNFLNTLYPNTFLNFSH
AVSLHLGNNKLQNIEGGAFLGLSALKQLHLNNNELKILRADTFPGIENLEYLQADYNLIKYIERGAFN
KLHKLKVLILNDNLISFLPDNIFRFASLTHLDIRGNRIQKLPYIGVLEHIGRVVELQLEDNPWNCSCD
LLPLKAWLENMPYNIYIGEAICETPSDLYGRLLKETNKQELCPMGTGSDFDVRILPPSQLENGYTTPN
GHTTQTSLHRLVTKPPKTTNPSKISGIVAGKALSNRNLSQIVSYQTRVPPLTPCPAPCFCKTHPSDLG
LSVNCQEKNIQSMSELIPKPLNAKKLHVNGNSIKDVDVSDFTDFEGLDLLHLGSNQITVIKGDVFHNL
TNLRRLYLNGNQIERLYPEIFSGLHNLQYLYLEYNLIKEISAGTFDSMPNLQLLYLNNNLLKSLPVYI
FSGAPLARLNLRNNKFMYLPVSGVLDQLQSLTQIDLEGNPWDCTCDLVALKLWVGKLSDGIVVKELKC
ETPVQFANIELKSLKNEILCPKLLNKPSAPFTSPAPTITFTTPLGPIRSPPGGPLE

| | | |
|---|---|---|
| NOV62d, CG98011-02 | SEQ ID NO: 1207 | 12654 bp |
| DNA Sequence | ORF Start: ATG at 19 | ORF Stop: TAG at 2647 |

CCATGTGTTCACTATGGTATGATTTACATACAAGTAATTTTTCAAGTAATGACCATTGAAAAAATGTT
TTCTTTTTATTTTTTAGATTATTTCTCTTTATTCAGAAGCATACAGTTGTTTGCTGATTGCAAGAAGA
TGTTTCTGTGGCTGTTTCTGATTTTGTCAGCCCTGATTTCTTCGACAAATGCAGATTCTGACATATCG
GTGGAAATTTGCAATGTGTGTTCCTGCGTGTCAGTTGAGAATGTGCTCTATGTCAACTGTGAGAAGGT
TTCAGTCTACAGACCAAATCAGCTGAAACCACCTTGGTCTAATTTTTATCACCTCAATTTCCAAAATA
ATTTTTTAAATATTCTGTATCCAAATACATTCTTGAATTTTTCACATGCAGTCTCCCTGCATCTGGGG
AATAATAAACTGCAGAACATTGAGGGAGGAGCCTTTCTTGGGCTCAGTGCATTAAAGCAGTTGCACTT
GAACAACAATGAATTAAAGATTCTCCGAGCTGACACTTTCCTTGGCATAGAGAACTTGGAGTATCTCC
AGGCTGACTACAATTTAATCAAGTATATTGAACGAGGAGCCTTCAATAAGCTCCACAAACTGAAAGTT
CTCATTCTTAATGACAATCTGATTTCATTCCTTCCTGATAATATTTTCCGATTCGCATCTTTGACCCA
TCTGGATATACGAGGGAACAGAATCCAGAAGCTCCCTTATATCGGGGTTCTGGAACACATTGGCCGTG
TCGTTGAATTGCAACTGGAAGATAACCCTTGGAACTGTAGCTGTGATTTATTGCCCTTAAAAGCTTGG
CTGGAGAACATGCCATATAACATTTACATAGGAGAAGCTATCTGTGAAACTCCCAGTGACTTATATGG
AAGGCTTTTAAAGAAACCAACAAACAAGAGCTATGATCCCATGGGCACCGGCAGTGATTTTGACGTGC
GCATCCTGCCTCCATCTCAGCTGGAAAATGGCTACACCACTCCCAATGGTCACACTACCCAAACATCT
TTACACAGATTAGTAACTAACCACCAAAAACAACAAATCACTTCCAAGATCTCTGGAATCGTTGCAGG
CAAAGCCCTCTCCAACCGCAATCTCAGTCAGATTGTGTCTTACCAAACAAGGGTGCCTCCTCTAACAC
CTTGCCCGGCACCTTGCTTCTGCAAAACACACCCTTCAGATTTGGGACTAAGTGTGAACTGCCAAGAG
AAAATATACAGTCTATGTCTGAACTGATACCGAAACCTTTAAAATGCGAAGAAGCTGCACGTCAATGG
CAATAGCATCAAGGATGTGGACGTATCAGACTTCACTGACTTTGAAGGACTGGATTTGCTTCATTTAG
GCAGCAATCAAATTACAGTGATTAAGGGAGACGTATTTCACAATCTCACTAATTTACGCAGGCTATAT
CTCAATGGCAATCAAATTGAGAGACTCTATCCTGAAATATTTTCAGGTCTTCATAACCTGCAGTATCT
GTATTTGGAATACAATTTGATTAAGGAAATCTCAGCAGGCACCTTTGACTCCATGCCAAATTTGCAGT
TACTGTACTTAAACAATAATCTCCTAAAGAGCCTGCCTGTTTACATCTTTTCCGGAGCACCCTTAGCT
AGACTGAACCTGAGGAACAACAAATTCATGTACCTGCCTGTCAGTGGGGTCCTTGATCAGTTGCAATC
TCTTACACAGATTGACTTGGAGGGCAACCCATGGGACTGTACTTGTGACTTGGTGGCATTAAAGCTGT
GGGTGGAGAAGTTGAGCGACGGGATTGTTGTGAAAGAACTGAAATGTGAGACGCCTGTTCAGTTTGCC
AACATTGAACTGAAGTCCCTCAAAAATGAAATCTTATGTCCCAAACTTTTAAATAAGCCGTCTGCACC
ATTCACAAGCCCTGCACCTGCCATTACATTCACCACTCCTTTGGGTCCCATTCGAAGTCCTCCTGGTG
GGCCAGTGCCTCTGTCTATTTTAATCTTAAGTATCTTAGTGGTCCTCATTTTAACGGTGTTTGTTGCT
TTTTGCCTTCTTGTTTTTGTCCTGCGACGCAACAAGAAACCCACAGTGAAGCACGAAGGCCTGGGGAA
TCCTGACTGTGGCTCCATGCAGCTGCAGCTAAGGAAGCATGACCACAAAACCAATAAAAAAGATGGAC
TGAGCACAGAAGCTTTCATTCCACAAACTATAGAACAGATGAGCAAGAGCCACACTTGTGGCTTGAAA
GAGTCAGAAACTGGGTTCATGTTTTCAGATCCTCCAGGACAGAAAGTTGTTATGAGAAATGTGGCCGA
CAAGGAGAAAGATTTATTACATGTAGATACCAGGAAGAGACTGAGCACAATTGATGAGCTGGATGAAT
TATTCCCTAGCAGGGATTCCAATGTGTTTATTCAGAATTTTCTTGAAAGCAAAAAGGAGTATAATAGC
ATAGGTGTCAGTGGCTTTGAGATCCGCTATCCAGAAAAACAACCAGACAAAAAAAGTAAGAAGTCACT
GATAGGTGGCAACCACAGTAAAATTGTTGTGGAACAAAGGAAGAGTGAGTATTTTGAACTGAAGGCGA
AACTGCAGAGTTCCCCTGACTACCTACAGGTCCTTGAGGAGCAAACAGCTTTGAACAAGATCTAGGTC
AT

| | | | |
|---|---|---|---|
| NOV62d, CG9801 -02 | | | |
| Protein Sequence | SEQ ID NO: 1208 | 876 aa | MW at 99167.6kD |

MIYIQVIFQVMTIEKMFSFYFLDYFSLFRSIQLFADCKKMFLWLFLILSALISSTNADSDISVEICNV
CSCVSVENVLYVNCEKVSVYRPNQLKPPWSNFYHLNFQNNFLNILYPNTFLNFSHAVSLHLGNNKLQN
IEGGAFLGLSALKQLHLNNNELKILRADTFLGIENLEYLQADYNLIKYIERGAFNKLHKLKVLILNDN
LISFLPDNIFRFASLTHLDIRGNRIQKLPYIGVLEHIGRWELQLEDNPWNCSCDLLPLKAAWLENMPY
NIYIGEAICETPSDLYGRLLKETNKQELCPMGTGSDFDVRILPPSQLENGYTTPNGHTTQTSLHRLVT
KPPKTTNPSKISGIVAGKALSNRNLSQIVSYQTRVPPLTPCPAPCFCKTHPSDLGLSVNCQEKNIQSM
SELIPKPLNAKKLHVNGNSIKDVDVSDFTDFEGLDLLHLGSNQITVIKGDVFHNLTNLRRLYLNGNQI
ERLYPEIFSGLHNLQYLYLEYNLIKEISAGTFDSMPNLQLLYLNNNLLKSLPVYIFSGAPLARLNLRN
NKFMYLPVSGVLDQLQSLTQIDLEGNPWDCTCDLVALKLWVEKLSDGIVVKELKCETPVQFANIELKS
LKNEILCPKLLNKPSAPFTSPAPAITFTTPLGPIRSPPGGPVPLSILILSILVVLILTVFVAFCLLVF
VLRRNKKPTVKHEGLGNPDCGSMQLQLRKHDHKTNKKDGLSTEAFIPQTIEQMSKSHTCGLKESETGF
MFSDPPGQKVTMRNVADKEKDLLHVDTRKRLSTIDELDELFPSRDSNVFIQNFLESKKEYNSIGVSGF
EIRYPEKQPDKKSKKSLIGGNHSKIVVEQRKSEYFELKAXLQSSPDYLQVLEEQTALNKI

| | | |
|---|---|---|
| NOV62e, CG98011-03 | SEQ ID NO: 1209 | 12469 bp |
| DNA Sequence | ORF Start: at 7 | ORF Stop: at 2464 |

GGATCCGATTCTGACATATCGGTGGAAATTTGCAATGTGTGTTCCTGCGTGTCAGTTGAGAATGTGCT
CTATGTCAACTGTGAGAAGGTTTCAGTCTACAGGCCAAATCAGCTGAAACCACCTTGGTCTAATTTTT
ATCACCTCAATTTCCAAAATAATTTTTTAAATATTCTGTATCCAAATACATTCTTGAATTTTTCACAT
GCAGTCTCCCTGCATCTGGGGAATAATAAACTGCAGAACATTGAGGGAGGAGCCTTTCTTGGGCTCAG
TGCATTAAAGCAGTTGCACTTGAACAACAATGAATTAAAGATTCTCCGAGCTGACACTTTCCTTGGCA
TAGAGAACTTGGAGTATCTCCAGGCTGACTACAATTTAATCMiGTATATTGAACGAGGAGCCTTCAAT
AAGCTCCACAAACTGAAAGTTCTCATTCTTAATGACAATCTGATTTCATTCCTTCCTGATAATATTTT
CCGATTCGCATCTTTGACCCATCTGGATATACGAGGGAACAGAATCCAGAAGCTCCCTTATATCGGGG
TTCTGGATCACATTGGCCGTGTCGTTGAATTGCAACTGGAAGATAACCCTTGGAACTGTAGCTGTGAT
TTATTGCCCTTAAAAGCTTGGCTGGAGAACATGCCATATAACATTTACATAGGAGAAGCTATCTGTGA
AACTCCCAGTGACCTATATGGAAGGCTTTTAAAAGAAACCAACAAACAAGAGCTATGTCCCATGGGCA
CCGGCAGTGATTTTGACGTGCGCATCCTGCCTCCATCTCAGCTGGAAAATGGCTACACCACTCCCAAT
GGTCACACTACCCAAACATCTTTACACAGATTAGTAACTAAACCACCAAAAACAACAAATCCTTCCAA
GATCTCTGGAATCGTTGCAGGCAAAGCCCTCTCCAACCGCAATCTCAGTCAGATTGTGTCTTACCAAA
CAAGGGTGCCTCCTCTAACACCTTGCCCGGCACCTTGCTTCTGCAAAACACACCCTTCAGATTTGGGA
CTAAGTGTGAACTGCCAAGAGAAAAATATACAGTCTATGTCTGAACTGATACCGAAACCTTTAAATGC
GAAGAAGCTGCACGTCAATGGCAATAGCATCAAGGATGTGGACGTATCAGACTTCACTGACTTTGAAG
GACTGGATTTGCTTCATTTAGGCAGCAATCAAATTACAGTGATTAAGGGAGACGTATTTCACAATCTC
ACTAATTTACGCAGGCTATATCTCAATGGCAATCAAATTGAGAGACTCTATCCTGAAATATTTTCAGG
TCTTCATAACCTGCAGTATCCGTATTTGGAATACAATTTGATTAAGGAAATCTCAGCAGGCACCTTTG
ACTCCATGCCAAATTTGCAGTTACTGTACTTAAACAATAATCTCCTAAAGAGCCTGCCTGTTTACATC
TTTTCCGGAGCACCCTTAGCTAGACTGAACCTGAGGAACAACAAATTCATGTACCTGCCTGTCAGTGG
GGTCCTTGATCAGTTGCAATCTCTTACACAGATTGACTTGGAGGGCAACCCATGGGACTGTACTTGTG
ACTTGGTGGCATTAAAGCTGTGGGTGGAGAAGTTGAGCGACGGGATTGTTGTGAAAGAACTGAAATGT
GAGACGCCTGTTCAGTTTGCCAACATTGAACTGAAGTCCCTCAAAAATGAAATCTTATGTCCCAAACT
TTTAAATAAGCCGTCTGCACCATTCACAAGCCCTGCACCTGCCATTACATTCACCACTCCTTTGGGTC
CCATTCGAAGTCCTCCTGGTGGGCCAGTGCCTCTGTCTATTTTAATCTTAAGTATCTTAGTGGTCCTC
ATTTTAACGGTGTTTGTTGCTTTTTGCCTTCTTGTTTTTGTCCTGCGACGCAACAAGAAACCCACAGT
GAAGCACGAAGGCCTGGGGAATCCTGACTGTGGCTCCATGCAGCTGCAGCTAAGGAAGCATGACCACA
AAACCAATAAAAAAGATGGACTGAGCACAGAAGCTTTCATTCCACAAACTATAGAACAGATGAGCAAG
AGCCACACTTGTGGCTTGAAAGAGTCAGAAACTGGGTTCATGTTTTCAGATCCTCCAGGACAGAAAGT
TGTTATGAGAAATGTGGCCGACAAGGAGAAAGATTTATTACATGTAGATACCAGGAAGAGACTGAGCA
CAATTGATGAGCTGGATGAATTATTCCCTAGCAGGGATTCCAATGTGTTTATTCAGAATTTTCTTGAA
AGCAAAAAGGAGTATAATAGCATAGGTGTCAGTGGCTTTGAGATCCGCTATCCAGAAAAACAACCAGA
CAAAAAAAGTAAGAAGTCACTGATAGGTGGCAACCACAGTAAAATTGTTGTGGAACAAAGGAAGAGTG
AGTATTTTGAACTGAAGGCGAAACTGCAGAGTTCCCCTGACTACCTACAGGTCCTTGAGGAGCAAACA
GCTTTGAACAAGATCCTCGAG

| | | | |
|---|---|---|---|
| NOV62e, CG98011-03 | | | |
| Protein Sequence | SEQ ID NO: 1210 | 819 aa | MW at 92291.4kD |

DSDISVEICNVCSCVSVENVLYVNCEKVSVYRPNQLKPPWSNFYHLNFQNNFLNILYPNTFLNFSHAV
SLHLGNNKLQNIEGGAFLGLSALKQLHLNNNELKILRADTFLGIENLEYLQADYNLIKYIERGAFNKL
HKLKVLILNDNLISFLPDNIFRFASLTHLDIRGNRIQKLPYIGVLEHIGRVVELQLEDNPWNCSCDLL
PLKAWLENMPYNIYIGEAICETPSDLYGRLLKETNKQELCPMGTGSDFDVRILPPSQLENGYTTPNGH
TTQTSLHRLVTKPPKTTNPSKISGIVAGKALSNRNLSQIVSYQTRVPPLTPCPAPCFCKTHPSDLGLS
VNCQEKNIQSMSELIPKPLNAKKLHVNGNSIKDVDVSDFTDFEGLDLLHLGSNQITVIKGDVFHNLTN
LRRLYLNGNQIERLYPEIFSGLHNLQYPYLEYNLIKEISAGTFDSMPNLQLLYLNNNLLKSLPVYIFS
GAPLARLNLRNNKFMYLPVSGVLDQLQSLTQIDLEGNPWDCTCDLVALKLWVEKLSDAGIWKELKCET
PVQFANIELKSLKNEILCPKLLNKPSAPFTSPAPAITFTTPLGPIRSPPGGPVPLSILILSILVVLIL
TVFVAFCLLVFVLRRNKKPTVKHEGLGNPDCGSMQLQLRKHDHKTNKKDGLSTEAFIPQTIEQMSKSH
TCGLKESETGFMFSDPPGQKVVMRNVADKEKDLLHVDTRKRLSTIDELDELFPSRDSNVFIQNFLESK
KEYNSIGVSGFEIRYPEKQPDKKSKKSLIGGNHSKIVVEQRKSEYFELKAKLQSSPDYLQVLEEQTAL
NKI

| | | |
|---|---|---|
| NOV62f, CG98011-04 | SEQ ID NO: 1211 | 1800 bp |
| DNA Sequence | ORF Start: at 7 | ORF Stop: at 1795 |

GGATCCGATTCTGACATATCGGTGGAAATTTGCAATGTGTGTTCCTGCGTGTCAGTTGAGAATGTGCT
CTATGTCAACTGTGAGAAGGTTTCAGTCTACAGACCAAATCAGCTGAAACCACCTTGGTCTAATTTTT
ATCACCTCAATTTCCAAAATAATTTTTTAAATATTCTGTATCCAAATACATTCTTGAATTTTTCACAT
GCAGTCTCCCTGCATCTGGGGAATAATAAACTGCAGAACATTGAGGGAGGAGCCTTTCTTGGGCTCAG
TGCATTAAAGCAGTTGCACTTGAACAACAATGAATTAAAGATTCTCCGAGCTGACACTTTCCCTGGCA
TAGAGAACTTGGAGTATCTCCAGGCTGACTACAATTTAATCAAGTATATTGAACGAGGAGCCTTCAAT
AAGCTCCACAAACTGAAAGTTCTCATTCTTAATGACAATCTGATTTCATTCCTTCCTGATAATATTTT
CCGATTCGCATCTTTGACCCATCTGGATATACGAGGGAACAGAATCCAGAAGCTCCCTTATATCGGGG
TTTCTGGACACATTGGCCGTGTCGTTGAATTGCAACTGGAAGATAACCCTTGGAACTGTAGCTGTGAT
TTATTGCCCTTAAAAGCTTGGCTGGAGAACATGCCATATAACATTTACATAGGAGAAGCTATCTGTGA
AACTCCCAGTGACTTATATGGAAGGCTTTTAAAAGAAACCAACAAACAAGAGCTATGTCCCATGGGCA
CCGGCAGTGATTTTGACGTGCGCATCCTGCCTCCATCTCAGCTGGAAAATGGCTACACCACTCCCAAT
GGTCACACTACCCAAACATCTTTACACAGATTAGTAACTAAACCACCAAAAACAACAAATCCTTCCAA
GATCTCTGGAATCGTTGCAGGCAAAGCCCTCTCCAACCGCAATCTCAGTCAGATTGTGTCTTACCAAA
CAAGGGTGCCTCCTCTAACACCTTGCCCGGCACCTTGCTTCTGCAAAACACACCCTTCAGATTTGGGA
CTAAGTGTGAACTGCCAAGAGAAAAATATACAGTCTATGTCTGAACTGATACCGAAACCTTTAAATGC
GATGAAGCTGCACGTCAATGGCAATAGCATCAAGGATGTGGACGTATCAGACTTCACTGACTTTGAAG
GACTGGATTTGCTTCATTTAGGCAGCAATCAAATTACAGTGATTAAGGGAGACGTATTTCACAATCTC
ACTAATTTACGCAGGCTATATCTCAATGGCAATCAAATTGAGAGACTCTATCCTGAAATATTTTCAGG
TCTTCATAACCTGCAGTATCTGTATTTGGAATACAATTTGATTAAGGAAATCTCAGCAGGCACCTTTG
ACTCCATGCCAAATTTGCAGTTACTGTACTTAAACAATAATCTCCTAAAGAGCCTGCCTGTTTACATC
TTTTCCGGAGCACCCTTAGCTAGACTGAACCTGAGGAACAACAAATTCATGTACCTGCCTGTCAGTGG
GGTCCTTGATCAGTTGCAATCTCTTACACAGATTGACTTGGAGGGCAACCCATGGGACTGTACTTGTG
ACTTGGTGGCATTAAAGCTGTGGGTGGGGAAGTTGAGCGACGGGATTGTTGTGAAAGAACTGAAATGT
GAGACGCCTGTTCAGTTTGCCAACATTGAACTGAAGTCCCTCAAAAATGAAATCTTATGTCCCAAACT
TTTAAATAAGCCGTCTGCACCATTCACAAGCCCTGCACCTACCATTACATTCACCACTCCTTTGGGTC
CCATTCGAAGTCCTCCTGGTGGGCCACTCGAG

| | | | |
|---|---|---|---|
| NOV62f, CG98011-04 | | | |
| Protein Sequence | SEQ ID NO: 1212 | 596 aa | MW at 66893.3kD |

DSDISVEICNVCSCVSVENVLYVNCEKVSVYRPNQLKPPWSNFYHLNFQNNFLNILYPNTFLNFSHAV
SLHLGNNKLQNIEGGAFLGLSALKQLHLNNNELKILRADTFPGIENLEYLQADYNLIKYIERGAFNKL
HKLKVLILNDNLISFLPDNIFRFASLTHLDIRGNRIQKLPYIGVLEHIGRWELQLEDANPWNCSCDLL
PLKWLENMPYNIYIGEAICETPSDLYGRLLKETNKQELCPMGTGSDFDVRILPPSQALENGYTTPNGH
TTQTSLHRLVTKPPKTTNPSKISGIVAGKALSNRNLSQIVSYQTRVPPLTPCPAPCFCKTHPSDLGLS
VNCQEKNIQSMSELIPKPLNAKKLHVNGNSIKDVDVSDFTDFEGLDLLHLGSNQITVIKGDVFHNLTN
LRRLYLNGNQIERLYPEIFSGLHNLQYLYLEYNLIKEISAGTFDSMPNLQLLYLNNNLLKSLPVYIFS
GAPLARLNLRNNKFMYLPVSGVLDQLQSLTQIDLEGNPWDCTCDLVALKLWVGKLSDGIVVKELKCET
PVQFANIELKSLKNEILCPKLLNKPSAPFTSPAPTITFTTPLGPIRSPPGGP

A ClustalW comparison of the above protein sequences yields the following sequence alignment shown in Table 62B.

TABLE 62B

Comparison of the NOV62 protein sequences.

| | |
|---|---|
| NOV62a | MIYIQVIFQVMTIEKMFSFYFLDYFSLFRSIQLFADCKKMFLWLFLILSALISSTNADSD |
| NOV62b | -------------------------------------------------------GSDSD |
| NOV62c | -------------------------------------------------------GSDSD |
| NOV62d | MIYIQVIFQVMTTEKMFSFYFLDYFSLFRSIQLFADCKKMFLWLFLILSALISSTNADSD |
| NOV62e | ---------------------------------------------------------DSD |
| NOV62f | ---------------------------------------------------------DSD |
| NOV62a | ISVEICNVCSCVSVENVLYVNCEKVSVYRPNQLKPPWSNFYHLNFQNNFLNILYPNTFLN |
| NOV62b | ISVEICNVCSCVSVENVLYVNCEKVSVYRPNQLKPPWSNFYHLNFQNNFLNILYPNTFLN |
| NOV62c | ISVEICNVCSCVSVENVLYVNCEKVSVYRPNQLKPPWSNFYHLNFQNNFLNILYPNTFLN |
| NOV62d | ISVEICNVCSCVSVENVLYVNCEKVSVYRPNQLKPPWSNFYHLNFQNNFLNILYPNTFLN |
| NOV62e | ISVEICNVCSCVSVENVLYVNCEKVSVYRPNQLKPPWSNFYHLNFQNNFLNILYPNTFLN |
| NOV62f | ISVEICNVCSCVSVENVLYVNCEKVSVYRPNQLKPPWSNFYHLNFQNNFLNILYPNTFLN |
| NOV62a | FSHAVSLHLGNNKLQNIEGGAFLGLSALKQLHLNNNELKILRADTFLGIENLEYLQADYN |
| NOV62b | FSHAVSLHLGNNKLQNIEGGAFLGLSTLKQLHLNNNELKILRADTFLGIENLEYLQADYN |
| NOV62c | FSHAVSLHLGNNKLQNIEGGAFLGLSALKQLHLNNNELKILRADTFPGIENLEYLQADYN |
| NOV62d | FSHAVSLHLGNNKLQNIEGGAFLGLSALKQLHLNNNELKILRADTFLGIENLEYLQADYN |
| NOV62e | FSHAVSLHLGNNKLQNIEGGAFLGLSALKQLHLNNNELKILRADTFLGIENLEYLQADYN |
| NOV62f | FSHAVSLHLGNNKLQNIEGGAFLGLSALKQLHLNNNELKILRADTFPGIENLEYLQADYN |
| NOV62a | LIKYIERGAFNKLHKLKVLILNDNLISFLPDNIFRFASLTHLDIRGNRIQKLPYIGVLEH |
| NOV62b | LIKYIERGAFNKLHKLKVLILNDNLISFLPDNIFRFASLTHLDIRGNRIQKLPYIGVLEH |
| NOV62c | LIKYIERGAFNKLHKLKVLILNDNLISFLPDNIFRFASLTHLDIRGNRIQKLPYIGVLEH |
| NOV62d | LIKYIERGAFNKLHKLKVLILNDNLISFLPDNIFRFASLTHLDIRGNRIQKLPYIGVLEH |
| NOV62e | LIKYIERGAFNKLHKLKVLILNDNLISFLPDNIFRFASLTHLDIRGNRIQKLPYIGVLEH |
| Nov62f | LIKYIERGAFNKLHKLKVLILNDNLISFLPDNIFRFASLTHLDIRGNRIQKLPYIGVLEH |
| NOV62a | IGRVVELQLEDNPWNCSCDLLPLKAWLENNPYNIYIGEAICETPSDLYGRLLKETNKQEL |
| NOV62b | IGRVVELQLEDNPWNCSCDLLPLKAWLENMPYNIYIGEAICETPSDLYGRLLKEThKQEL |
| NOV62c | IGRVVELQLEDNPWNCSCDLLPLKAWLENMPYNIYIGEAICETPSDLYGRLLKETNKQEL |
| NOV62d | IGRVVELQLEDNPWNCSCDLLPLKAWLENNPYNIYIGEAICETPSDLYGRLLKETNKQEL |
| NOV62e | IGRVVELQLEDNPWNCSCDLLPLKAWLENMPYNIYIGEAICETPSDLYGRLLKETNKQEL |
| NOV62f | IGRVVELQLEDNPWNCSCDLLPLKAWLENNPYNIYIGEAICETPSDLYGRLLKETNKQEL |
| NOV62a | CPMGTGSDFDVRILPPSQLENGYTTPNGHTTQTSLHRLVTKPPKTTNPSKISGIVAGKAL |
| NOV62b | CPMGTGSDFDVRTLPPSQLENGYTTPNGHTTQTSLHRLVTKPPKTTNPSKISGIVAGKAL |
| NOV62c | CPMGTGSDFDVRILPPSQLENGYTTPNGHTTQTSLHRLVTKPPKTTNPSKISGIVAGKAL |
| NOV62d | CPMGTGSDFDVRILPPSQLENGYTTPNGHTTQTSLHRLVTKPPKTTNPSKISGIVAGKAL |
| NOV62e | CPMGTGSDFDVRILPPSQLENGYTTPNGHTTQTSLHRLVTKPPKTTNPSKISGIVAGKAL |
| NOV62f | CPMGTGSDFDVRILPPSQLENGYTTPNGHTTQTSLHRLVTKPPKTTNPSKISGIVAGKAL |
| NOV62a | SNRNLSQIVSYQTRVPPLTPCPAPCFCKTHPSDLGLSVNCQEKNIQSMSELIPKPLNAKK |
| NOV62b | SNRNLSQIVSYQTRVPPLTPCPAPCFCKTHPSDLGLSVNCQEKNIQSMSELIPKPLNAKK |
| NOV62c | SNRNLSQIVSYQTRVPPLTPCPAPCFCKTHPSDLGLSVNCQEKNTQSMSELIPKPLNAKK |
| NOV62d | SNRNLSQIVSYQTRVPPLTPCPAPCFCKTHPSDLGLSVNCQEKNIQSMSELIPKPLNAKK |
| NOV62e | SNRNLSQIVSYQTRVPPLTPCPAPCFCKTHPSDLGLSVNCQEKNIQSMSELIPKPLNAKK |
| NOV62f | SNRNLSQIVSYQTRVPPLTPCPAPCFCKTHPSDLGLSVNCQEKNIQSMSELIPKPLNAKK |
| NOV62a | LHVNGNSIKDVDVSDFTDFEGLDLLHLGSNQITVIKGDVFHNLTNLRRLYLNGNQIERLY |
| NOV62b | LHVNGNSIKDVDVSDFTDFEGLDLLHLGSNQITVIKGDVFHNLTNLRRLYLNGNQIERLY |
| NOV62c | LHVNGNSIKDVDVSDFTDFEGLDLLHLGSNQITVIKGDVFHNLTNLRRLYLNGNQIERLY |
| NOV62d | LHVNGNSIKDVDVSDFTDFEGLDLLHLGSNQITVIKGDVFHNLTNLRRLYLNGNQIERLY |
| NOV62e | LHVNGNSIKDVDVSDFTDFEGLDLLHLGSNQITVIKGDVFHNLTNLRRLYLNGNQIERLY |
| NOV62f | LHVNGNSIKDVDVSDFTDFEGLDLLHLGSNQITVIKGDVFHNLTNLRRLYLNGNQIERLY |
| NOV62a | PEIFSGLHNLQYLYLEYNLIKEISAGTFDSMPNLQLLYLNNNLLKSLPVYIFSGAPLARL |
| NOV62b | PEIFSGLHNLQYLYLEYNLIKEISAGTFDSMPNLQLLYLSNNLLKSLPVYIFSGAPLARL |
| NOV62c | PEIFSGLHNLQYLYLEYNLIKEISAGTFDSMPNLQLLYLNNNLLKSLPVYIFSGAPLARL |
| NOV62d | PEIFSGLHNLQYLYLEYNLIKEISAGTFDSMPNLQLLYLNNNLLKSLPVYIFSGAPLARL |
| NOV62e | PEIFSGLHNLQYPYLEYNLIKEISAGTFDSMPNLQLLYLNNNLLKSLPVYIFSGAPLARL |
| NOV62f | PEIFSGLHNLQYLYLEYNLIKEISAGTFDSMPNLQLLYLNNNLLKSLPVYIFSGAPLARL |
| NOV62a | NLRNNKFMYLPVSGVLDQLQSLTQIDLEGNPWDCTCDLVALKLWVEKLSDGIVVKELKCE |
| NOV62b | NLRNNKFMYLPVSGVLDQLQSLTQIDLEGSPWDYTCDLVALKLWVEKLSDGIVVKELKCE |
| NOV62c | NLRNNKFMYLPVSGVLDQLQSLTQIDLEGNPWDCTCDLVALKLWVGKLSDGIVVKELKCE |
| NOV62d | NLRNNKFMYLPVSGVLDQLQSLTQIDLEGNPWDCTCDLVALKLWVEKLSDGIVVKELKCE |
| NOV62e | NLRNNKFMYLPVSGVLDQLQSLTQTDLEGNPWDCTCDLVALKLWVEKLSDGIVVKELKCE |
| NOV62f | NLRNNKFMYLPVSGVLDQLQSLTQIDLEGNPWDCTCDLVALKLWVGKLSDGIVVKELKCE |
| NOV62a | TPVQFANIELKSLKNEILCPKLLNKPSAPFTSPAPATTFTTPLGPIRSPPGGPVPLSILI |
| NOV62b | TPVQFTNIELKSLKNEILCPKLLNKPSAPFTSPAPATTFTTPLGPIRSPPGGPLE----- |
| NOV62c | TPVQFANIELKSLKNEILCPKLLNKPSAPFTSPAPTITFTTPLGPIRSPPGGPLE----- |
| NOV62d | TPVQFANIELKSLKNEILCPKLLNKPSAPFTSPAPAITFTTPLGPIRSPPGGPVPLSILI |
| NOV62e | TPVQFANIELKSLKNEILCPKLLNKPSAPFTSPAPAITFTTPLGPIRSPPGGPVPLSILI |
| NOV62f | TPVQFANIELKSLKNETLCPKLLNKPSAPFTSPAPTITFTTPLGPIRSPPGGP------- |
| NOV62a | LSILVVLILTVFVAFCLLVFVLRRNKKPTVKHEGLGNPDCGSMQLQLRKHDHKTNKKDGL |
| NOV62b | ------------------------------------------------------------ |
| NOV62c | ------------------------------------------------------------ |
| NOV62d | LSILVVLILTVFVAFCLLVFVLRRNKKPTVKHEGLGNPDCGSMQLQLRKHDHKTNKKDGL |
| NOV62e | LSILVVLILTVFVAFCLLVFVLRRNKKPTVKHEGLGNPDCGSMQLQLRKHDHKTNKKDGL |
| NOV62f | ------------------------------------------------------------ |
| NOV62a | STEAFIPQTIEQMSKSHTCGLKESETGFMFSDPPGQKVVMRNVADKEKDLLHVDTRKRLS |
| NOV62b | ------------------------------------------------------------ |
| NOV62c | ------------------------------------------------------------ |
| NOV62d | STEAFIPQTIEQMSKSHTCGLKESETGFMFSDPPGQKVVMRNVADKEKDLLHVDTRKRLS |
| NOV62e | STEAFIPQTIEQMSKSHTCGLKESETGFMFSDPPGQKVVMRNVADKEKDLLHVDTRKRLS |
| NOV62f | ------------------------------------------------------------ |
| NOV62a | TIDELDELFPSRDSNVPIQNFLESKKEYNSIGVSGFETRYPEKQPDKKSKKSLIGGNHSK |
| NOV62b | ------------------------------------------------------------ |
| NOV62c | ------------------------------------------------------------ |
| NOV62d | TIDELDELFPSRDSNVFIQNFLESKKEYNSIGVSGFETRYPEKQPDKKSKKSLIGGNHSK |
| NOV62e | TIDELDELFPSRDSNVFIQNFLESKKEYNSIGVSGFEIRYPEKQPDKKSKKSLIGGNHSK |
| NOV62f | ------------------------------------------------------------ |
| NOV62a | IVVEQRKSEYFELKAKLQSSPDYLQVLEEQTALNKI |
| NOV62b | ------------------------------------ |
| NOV62c | ------------------------------------ |
| NOV62d | IVVEQRKSEYFELKAKLQSSPDYLQVLEEQTALNKI |
| NOV62e | IVVEQRKSEYFELKAKLQSSPDYLQVLEEQTALNKI |
| NOV62f | ------------------------------------ |
| NOV62a | (SEQ ID NO: 1202) |
| NOV62b | (SEQ ID NO: 1204) |
| NOV62c | (SEQ ID NO: 1206) |
| NOV62d | (SEQ ID NO: 1208) |
| NOV62e | (SEQ ID NO: 1210) |
| NOV62f | (SEQ ID NO: 1212) |

Further analysis of the NOV62a protein yielded the following properties shown in Table 62C.

TABLE 62C
Protein Sequence Properties NOV62a

| | |
|---|---|
| SignalP analysis: | Cleavage site between residues 58 and 59 |
| PSORT II analysis: | |

| PSG: | a new signal peptide prediction method | |
|---|---|---|
| | N-region: | length 0; pos. chg 0; neg. chg 0 |
| | H-region: | length 13; peak value 8.16 |
| | PSG score: | 3.76 |

| | |
|---|---|
| GvH: | von Heijne's method for signal seq. recognition |
| | GvH score (threshold: −2.1): 2.51 |
| | possible cleavage site: between 57 and 58 |

>>> Seems to have a cleavable signal peptide (1 to 57)

| ALOM: | Klein et al's method for TM region allocation | | |
|---|---|---|---|
| | Init position for calculation: 58 | | |
| | Tentative number of TMS(s) for the threshold 0.5: 1 | | |
| | Number of TMS(s) for threshold 0.5: 1 | | |
| | INTEGRAL | Likelihood = | Transmembrane 663-679 |
| | | −16.61 | |

| | |
|---|---|
| PERIPHERAL | Likelihood = 2.92 (at 578) |

| | |
|---|---|
| | ALOM score: −16.61 (number of TMSs: 1) |
| MTOP: | Prediction of membrane topology (Hartmann et al.) |
| | Center position for calculation: 28 |
| | Charge difference: 1.0 C(2.0)-N(1.0) |
| | C > N: C-terminal side will be inside |

>>>Caution: Inconsistent mtop result with signal peptide
>>> membrane topology: type 1a (cytoplasmic tail 680 to 876)

| MITDISC: | discrimination of mitochondrial targeting seq | | | |
|---|---|---|---|---|
| | R content: | 0 | Hyd Moment(75): | 4.59 |
| | Hyd Moment(95): | 1.85 | G content: | 0 |
| | D/E content: | 2 | S/T content: | 2 |

| | |
|---|---|
| | Score: −7.15 |
| Gavel: | prediction of cleavage sites for mitochondrial preseq |
| | cleavage site motif not found |

NUCDISC: discrimination of nuclear localization signals

| | |
|---|---|
| | pat4: none |
| | pat7: PDKKSKK (4) at 825 |
| | bipartite: none |
| | content of basic residues: 10.4% |
| | NLS Score: −0.13 |
| KDEL: | ER retention motif in the C-terminus: none |

ER Membrane Retention Signals:

KKXX-like motif in the C-terminus: ALNK

SKL: peroxisomal targeting signal in the C-terminus: NKI
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: found

LL at 770

checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 55.5 |
| COIL: | Lupas's algorithm to detect coiled-coil regions |
| | total: 0 residues |

Final Results (k = 9/23):

44.4%: endoplasmic reticulum
22.2%: Golgi
11.1%: plasma membrane
11.1%: vesicles of secretory system
11.1%: extracellular, including cell wall

>> prediction for CG98011-01 is end (k = 9)

A search of the NOV62a protein against the Geneseq database, a proprietary database that contains sequences published in patents and patent publication, yielded several homologous proteins shown in Table 62D.

TABLE 62D
Geneseq Results for NOV62a

| | | NOV62a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE18209 | Human MOL2 protein - Homo sapiens, | 1 . . . 876 | 876/876 (100%) | 0.0 |
| | 876 aa. [WO200206339-A2, 24-JAN-2002] | 1 . . . 876 | 876/876 (100%) | |
| AAG67510 | Amino acid sequence of a human | 11 . . . 876 | 866/866 (100%) | 0.0 |
| | secreted polypeptide - Homo sapiens, | 1 . . . 866 | 866/866 (100%) | |
| | 866 aa. [WO200166690-A2, 13-SEP-2001] | | | |
| AAE22221 | Human toll like receptor like molecule 4 | 40 . . . 876 | 837/837 (100%) | 0.0 |
| | (TLR-L4) protein - Homo sapiens, 837 | 1 . . . 837 | 837/837 (100%) | |
| | aa. [WO200220569-A2, 14-MAR-2002] | | | |
| ABJ01959 | Human IGFALS similar protein SEQ ID | 40 . . . 871 | 832/832 (100%) | 0.0 |
| | No 660 - Homo sapiens, 832 aa. | 1 . . . 832 | 832/832 (100%) | |
| | [WO200216593-A2, 28-FEB-2002] | | | |
| ABG36257 | Human peptide encoded by genome- | 24 . . . 598 | 575/575 (100%) | 0.0 |
| | derived single exon probe SEQ ID | 1 . . . 575 | 575/575 (100%) | |
| | 25922 - Homo sapiens, 575 aa. | | | |
| | [WO200186003-A2, 15-NOV-2001] | | | |

In a BLAST search of public sequence databases, the NOV62a protein was found to have homology to the proteins shown in the BLASTP data in Table 62E.

TABLE 62E
Public BLASTP Results for NOV62a

| | | NOV62a | Identities/ | |
|---|---|---|---|---|
| Protein | | Residues/ | Similarities for | |
| Accession | | Match | the Matched | Expect |
| Number | Protein/Organism/Length | Residues | Portion | Value |
| BAC67207 | Neuronal transmembrane protein | 11 . . . 876 | 840/866 (96%) | 0.0 |
| | Slitrk4 - Mus musculus (Mouse), 866 | 1 . . . 866 | 857/866 (97%) | |
| | aa. | | | |
| Q8IW52 | Similar to KIAA0848 protein - Homo | 40 . . . 876 | 837/837 (100%) | 0.0 |
| | sapiens (Human), 837 aa. | 1 . . . 837 | 837/837 (100%) | |
| Q8BWB0 | Hypothetical leucine-rich repeat | 40 . . . 876 | 814/837 (97%) | 0.0 |
| | cysteine-containing subfamily/ | 1 . . . 837 | 829/837 (98%) | |
| | cysteine-rich flanking region - Mus | | | |
| | musculus (Mouse), 837 aa. | | | |
| Q8C056 | Hypothetical leucine-rich repeat | 40 . . . 876 | 813/837 (97%) | 0.0 |
| | cysteine-containing subfamily/ | 1 . . . 837 | 828/837 (98%) | |
| | cysteine-rich flanking region - Mus | | | |
| | musculus (Mouse), 837 aa. | | | |
| Q8BYG4 | Hypothetical leucine-rich repeat | 40 . . . 821 | 761/782 (97%) | 0.0 |
| | cysteine-containing subfamily/ | 1 . . . 782 | 775/782 (98%) | |
| | cysteine-rich flanking region - Mus | | | |
| | musculus (Mouse), 782 aa (fragment). | | | |

PFam analysis predicts that the NOV62a protein contains the domains shown in the Table 62F.

TABLE 62F
Domain Analysis of NOV62a

| | | Identities/ | |
|---|---|---|---|
| | | Similarities for | |
| Pfam | NOV62a | the Matched | |
| Domain | Match Region | Region | Expect Value |
| LRR | 218 . . . 239 | 8/25 (32%) | 0.24 |
| | | 19/25 (76%) | |
| LRRCT | 252 . . . 302 | 18/54 (33%) | 9.7e−11 |
| | | 35/54 (65%) | |
| LRRNT | 380 . . . 413 | 11/36 (31%) | 0.0039 |
| | | 24/36 (67%) | |
| LRR | 441 . . . 464 | 9/25 (36%) | 0.31 |
| | | 16/25 (64%) | |
| LRR | 465 . . . 488 | 13/25 (52%) | 0.0072 |
| | | 23/25 (92%) | |
| LRR | 489 . . . 512 | 6/25 (24%) | 0.015 |
| | | 23/25 (92%) | |
| LRR | 513 . . . 536 | 12/25 (48%) | 0.03 |
| | | 19/25 (76%) | |
| LRRCT | 570 . . . 620 | 14/54 (26%) | 8.2e−06 |
| | | 35/54 (65%) | |

### Example B

### Sequencing Methodology and Identification of NOVX Clones

1. GeneCalling™ Technology: This is a proprietary method of performing differential gene expression profiling between two or more samples developed at CuraGen and described by Shimkets, et al., “Gene expression analysis by transcript profiling coupled to a gene database query” Nature Biotechnology 17:198-803 (1999). cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then digested with up to as many as 120 pairs of restriction enzymes and pairs of linker-adaptors specific for each pair of restriction enzymes were ligated to the appropriate end. The restriction digestion generates a mixture of unique cDNA gene fragments. Limited PCR amplification is performed with primers homologous to the linker adapter sequence where one primer is biotinylated and the other is fluorescently labeled. The doubly labeled material is isolated and the fluorescently labeled single strand is resolved by capillary gel electrophoresis. A computer algorithm compares the electropherograms from an experimental and control group for each of the restriction digestions. This and additional sequence-derived information is used to predict the identity of each differentially expressed gene fragment using a variety of genetic databases. The identity of the gene fragment is confirmed by additional, gene-specific competitive PCR or by isolation and sequencing of the gene fragment.

2. SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

3. PathCalling™ Technology: The NOVX nucleic acid sequences are derived by laboratory screening of cDNA library by the two-hybrid approach. cDNA fragments covering either the full length of the DNA sequence, or part of the sequence, or both, are sequenced. In silico prediction was based on sequences available in CuraGen Corporation's proprietary sequence databases or in the public human sequence databases, and provided either the full length DNA sequence, or some portion thereof.

The laboratory screening was performed using the methods summarized below:

cDNA libraries were derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then directionally cloned into the appropriate two-hybrid vector (Gal4-activation domain (Gal4-AD) fusion). Such cDNA libraries as well as commercially available cDNA libraries from Clontech (Palo Alto, Calif.) were then transferred from E. coli into a CuraGen Corporation proprietary yeast strain (disclosed in U.S. Pat. Nos. 6,057,101 and 6,083,693, incorporated herein by reference in their entireties).

Gal4-binding domain (Gal4-BD) fusions of a CuraGen Corportion proprietary library of human sequences was used to screen multiple Gal4-AD fusion cDNA libraries resulting in the selection of yeast hybrid diploids in each of which the Gal4-AD fusion contains an individual cDNA. Each sample was amplified using the polymerase chain reaction (PCR) using non-specific primers at the cDNA insert boundaries. Such PCR product was sequenced; sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: the cDNA fragment derived by the screening procedure, covering the entire open reading frame is, as a recombinant DNA, cloned into pACT2 plasmid (Clontech) used to make the cDNA library. The recombinant plasmid is inserted into the host and selected by the yeast hybrid diploid generated during the screening procedure by the mating of both CuraGen Corporation proprietary yeast strains N106′ and YULH (U.S. Pat. Nos. 6,057,101 and 6,083,693).

4. RACE: Techniques based on the polymerase chain reaction such as rapid amplification of cDNA ends (RACE), were used to isolate or complete the predicted sequence of the cDNA of the invention. Usually multiple clones were sequenced from one or more human samples to derive the sequences for fragments. Various human tissue samples from different donors were used for the RACE reaction. The sequences derived from these procedures were included in the SeqCalling Assembly process described in preceding paragraphs.

5. Exon Linking: The NOVX target sequences identified in the present invention were subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma - Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

6. Physical Clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The PCR product derived by exon linking, covering the entire open reading frame, was cloned into the pCR2.1 vector from Invitrogen to provide clones used for expression and screening purposes.

### Example C

### Quantitative Expression Analysis of Clones in Various Cells and Tissues

The quantitative expression of various clones was assessed using microfiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an enphasis on metabolic diseases), AI_comprehensive_panel (containing normal tissue and samples from autoinflammatory diseases), Panel CNSD.01 (containing samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, □-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 □g of total RNA were performed in a volume of 20 □l and incubated for 60 minutes at 42° C. This reaction can be scaled up to 50 □g of total RNA in a final volume of 100 □l. sscDNA samples are then normalized to reference nucleic acids as described previously, using 1×TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tm=59° C., maximum primer difference=2° C., probe does not have 5′ G, probe Tₘ must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100. CT values below 28 indicate high expression, CT values between 28 and 32 indicate moderate expression, and CT values between 32 and 35 indicate low expression. CT values above 35 reflect levels of expression that are too low to be reliably measured.

When working with sscDNA samples, normalized sscDNA was used as described previously for RNA samples. PCR reactions containing one or two sets of probe and primers were set up as described previously, using 1×TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:
ca.=carcinoma,
*=established from metastasis,
met=metastasis,
s cell var=small cell variant,
non-s=non-sm=non-small,
squam=squamous,
pl. elf=pl effusion=pleural effusion,
glio=glioma,
astro=astrocytoma, and
neuro=neuroblastoma.

GENERAL_SCREENING_PANEL_V1.4, V1.5, V1.6 AND 1.7

The plates for Panels 1.4, 1.5, 1.6 and 1.7 include 2 control wells (genomic DNA control and chemistry control) and 88 to 94 wells containing cDNA from various samples. The samples in Panels 1.4, 1.5, 1.6 and 1.7 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panels 1.4, 1.5, 1.6 and 1.7 are widely available through the American Type Culture Collection (ATCC), a repository for cultured cell lines, and were cultured using the conditions recommended by the ATCC. The normal tissues found on Panels 1.4, 1.5, 1.6 and 1.7 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D, 2.2, 2.3, and 2.4

The plates for Panels 2D, 2.2, 2.3 and 2.4 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI) or from Ardais or Clinomics). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI/CHTN/Ardais/Clinomics). Unmatched RNA samples from tissues without malignancy (normal tissues) were also obtained from Ardais or Clinomics. This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

HASS Panel V 1.0

The HASS panel v 1.0 plates are comprised of 93 cDNA samples and two controls. Specifically, 81 of these samples are derived from cultured human cancer cell lines that had been subjected to serum starvation, acidosis and anoxia for different time periods as well as controls for these treatments, 3 samples of human primary cells, 9 samples of malignant brain cancer (4 medulloblastomas and 5 glioblastomas) and 2 controls. The human cancer cell lines are obtained from ATCC (American Type Culture Collection) and fall into the following tissue groups: breast cancer, prostate cancer, bladder carcinomas, pancreatic cancers and CNS cancer cell lines. These cancer cells are all cultured under standard recommended conditions. The treatments used (serum starvation, acidosis and anoxia) have been previously published in the scientific literature. The primary human cells were obtained from Clonetics (Walkersville, Md.) and were grown in the media and conditions recommended by Clonetics. The malignant brain cancer samples are obtained as part of a collaboration (Henry Ford Cancer Center) and are evaluated by a pathologist prior to CuraGen receiving the samples. RNA was prepared from these samples using the standard procedures. The genomic and chemistry control wells have been described previously.

ARDAIS Panel V 1.0

The plates for ARDAIS panel v 1.0 generally include 2 control wells and 22 test samples composed of RNA isolated from human tissue procured by surgeons working in close cooperation with Ardais Corporation. The tissues are derived from human lung malignancies (lung adenocarcinoma or lung squamous cell carcinoma) and in cases where indicated many malignant samples have “matched margins” obtained from noncancerous lung tissue just adjacent to the tumor. These matched margins are taken from the tissue surrounding (i.e. immediately proximal) to the zone of surgery (designated “NAT”, for normal adjacent tissue) in the results below. The tumor tissue and the “matched margins” are evaluated by independent pathologists (the surgical pathologists and again by a pathologist at Ardais). Unmatched malignant and non-malignant RNA samples from lungs were also obtained from Ardais. Additional information from Ardais provides a gross histopathological assessment of tumor differentiation grade and stage. Moreover, most samples include the original surgical pathology report that provides information regarding the clinical state of the patient.

ARDAIS Prostate V 1.0

The plates for ARDAIS prostate 1.0 generally include 2 control wells and 68 test samples composed of RNA isolated from human tissue procured by surgeons working in close cooperation with Ardais Corporation. The tissues are derived from human prostate malignancies and in cases where indicated malignant samples have “matched margins” obtained from noncancerous prostate tissue just adjacent to the tumor. These matched margins are taken from the tissue surrounding (i.e. immediately proximal) to the zone of surgery (designated “NAT”, for normal adjacent tissue) in the results below. The tumor tissue and the “matched margins” are evaluated by independent pathologists (the surgical pathologists and again by a pathologist at Ardais). RNA from unmatched malignant and non-malignant prostate samples were also obtained from Ardais. Additional information from Ardais provides a gross histopathological assessment of tumor differentiation grade and stage. Moreover, most samples include the original surgical pathology report that provides information regarding the clinical state of the patient.

ARDAIS Kidney V 1.0

The plates for ARDAIS kidney 1.0 generally include 2 control wells and 44 test samples composed of RNA isolated from human tissue procured by surgeons working in close cooperation with Ardais Corporation. The tissues are derived from human prostate malignancies and in cases where indicated malignant samples have “matched margins” obtained from noncancerous prostate tissue just adjacent to the tumor. These matched margins are taken from the tissue surrounding (i.e. immediately proximal) to the zone of surgery (designated “NAT”, for normal adjacent tissue) in the results below. The tumor tissue and the “matched margins” are evaluated by independent pathologists (the surgical pathologists and again by a pathologist at Ardais). RNA from unmatched malignant and non-malignant prostate samples were also obtained from Ardais. Additional information from Ardais provides a gross histopathological assessment of tumor differentiation grade and stage. Moreover, most samples include the original surgical pathology report that provides information regarding the clinical state of the patient.

Panel 3D and 3.1 and 3.2

The plates of Panel 3D, 3.1, and 3.2 are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemnias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D, 3.1, 3.2, 1, 1.1., 1.2, 1.3D, 1.4, 1.5, and 1.6 are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL- 13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/mil in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μm non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/fffl anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and EL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (4 ng/ml). IL-12 (5 ng/ml) and anti-IL4 (1 □g/ml) were used to direct to Th1, while IL-4 (5 ng/ml) and anti-IFN gamma (1 □g/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 □g/ml) to prevent apoptosis. After 4-5 days, the Th1, Th2 and Tr1 lymphocytes were washed and then expanded again with IL-2 for 4-7 days. Activated Th1 and Tb2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37 degrees C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees C.

AI_comprehensive Panel_v1.0

The plates for AI_comprehensive_panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:
AI=Autoimmunity
Syn=Synovial
Normal=No apparent disease
Rep22/Rep20=individual patients
RA=Rheumatoid arthritis
   Backus=From Backus Hospital
   OA=Osteoarthritis
(SS) (BA) (MF)=Individual patients
Adj=Adjacent tissue
Match control=adjacent tissues
-M=Male
-F=Female
COPD=Chronic obstructive pulmonary disease

AI.05 Chondrosarcoma

The AI.05 chondrosarcoma plates are comprised of SW1353 cells that had been subjected to serum starvation and treatment with cytokines that are known to induce MMP (1, 3 and 13) synthesis (eg. IL1 beta). These treatments include: IL-1□ (10 ng/ml), IL-1□+ TNF-□ (50 ng/ml), IL-1□+ Oncostatin (50 ng/ml) and PMA(100 ng/ml). The SW1353 cells were obtained from the ATCC (American Type Culture Collection) and were all cultured under standard recommended conditions. The SW1353 cells were plated at 3×10⁵ cells/ml (in DMEM medium-10% FBS) in 6-well plates. The treatment was done in triplicate, for 6 and 18 h. The supernatants were collected for analysis of MMP 1, 3 and 13 production and for RNA extraction. RNA was prepared from these samples using the standard procedures.

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

| | |
|---|---|
| Patient 2 | Diabetic Hispanic, overweight, not on insulin |
| Patient 7-9 | Nondiabetic Caucasian and obese (BMI > 30) |
| Patient 10 | Diabetic Hispanic, overweight, on insulin |
| Patient 11 | Nondiabetic African American and overweight |
| Patient 12 | Diabetic Hispanic on insulin |

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2, 1999: 143-147. Clonetics provided Trizol lysates or frozen pellets suitable for mRNA isolation and ds cDNA production. A general description of each donor is as follows:

| | | | |
|---|---|---|---|
| Donor 2 and 3 | U | Mesenchymal | Undifferentiated Adipose |
| | | Stem cells | |
| Donor 2 and 3 | AM | Adipose | Adipose Midway Differentiated |
| Donor 2 and 3 | AD | Adipose | Adipose Differentiated |

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:
GO Adipose=Greater Omentum Adipose
SK=Skeletal Muscle
UT=Uterus
PL=Placenta
AD=Adipose Differentiated
AM=Adipose Midway Differentiated
U=Undifferentiated Stem Cells

Human Metabolic RTQ-PCR Panel

The plates for the Human Metabolic RTQ-PCR Panel include two control wells (genomic DNA control and chemistry control) and 211 cDNAs isolated from human tissues and cell lines with an emphasis on metabolic diseases. This panel is useful for establishing the tissue and cellular expression profiles for genes believed to play a role in the etiology and pathogenesis of obesity and/or diabetes and to confirm differential expression of such genes derived from other methods.

Metabolic tissues were obtained from patients enrolled in the CuraGen Gestational Diabetes study and from autopsy tissues from Type II diabetics and age, sex and race-matched control patients. One or more of the following were used to characterize the patients: body mass index [BMI=wt (kg)/ht (m²)], serum glucose, HgbA1c. Cell lines used in this panel are widely available through the American Type Culture Collection (ATCC), a repository for cultured cell lines. RNA from human Pancreatic Islets was also obtained.

In the Gestational Diabetes study, subjects are young (18-40 years), otherwise healthy women with and without gestational diabetes undergoing routine (elective) Caesarian section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted, and then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus), and subcutaneous adipose. Patient descriptions are as follows:

| | |
|---|---|
| Patient 7 | Non-diabetic Caucasian and obese |
| Patient 8 | Non-diabetic Caucasian and obese |
| Patient 12 | Diabetic Caucasian with unknown BMI and on insulin |
| Patient 13 | Diabetic Caucasian, overweight, not on insulin |
| Patient 15 | Diabetic Caucasian, obese, not on insulin |
| Patient 17 | Diabetic Caucasian, normal weight, not on insulin |
| Patient 18 | Diabetic Hispanic, obese, not on insulin |
| Patient 19 | Non-diabetic Caucasian and normal weight |
| Patient 20 | Diabetic Caucasian, overweight, and on insulin |
| Patient 21 | Non-diabetic Caucasian and overweight |
| Patient 22 | Diabetic Caucasian, normal weight, on insulin |
| Patient 23 | Non-diabetic Caucasian and overweight |
| Patient 25 | Diabetic Caucasian, normal weight, not on insulin |
| Patient 26 | Diabetic Caucasian, obese, on insulin |
| Patient 27 | Diabetic Caucasian, obese, on insulin |

Total RNA was isolated from metabolic tissues of 12 Type II diabetic patients and 12 matched control patients included hypothalamus, liver, pancreas, small intestine, psoas muscle, diaphragm muscle, visceral adipose, and subcutaneous adipose. The diabetics and non-diabetics were matched for age, sex, ethnicity, and BMI where possible.

The panel also contains pancreatic islets from a 22 year old male patient (with a BMI of 35) obtained from the Diabetes Research Institute at the University of Miami School of Medicine. Islet tissue was processed to total RNA at CuraGen.

Cell lines used in this panel are widely available through the American Type Culture Collection (ATCC), a repository for cultured cell lines, and were cultured at an outside facility. The RNA was extracted at CuraGen according to CuraGen protocols. All samples were then processed at CuraGen to produce single stranded cDNA.

In the labels used to identify tissues in the Human Metabolic panel, the following abbreviations are used:

Panel CNSD.01

| | |
|---|---|
| Pl = | placenta |
| Go = | greater omentum |
| Sk = | skeletal muscle |
| Ut = | uterus |
| CC = | Caucasian |
| HI = | Hispanic |
| AA = | African American |
| AS = | Asian |
| Diab = | Type II diabetic |
| Norm = | Non-diabetic |
| Overwt = | Overweight; med BMI |
| Obese = | Hi BMI |
| Low BM = | 20-25 |
| Med BM = | 26-30 |
| Hi BMI = | Greater than 30 |
| M = | Male |
| # = | Patient identifier |
| Vis. = | Visceral |
| SubQ = | Subcutaneous |

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:
PSP=Progressive supranuclear palsy
Sub Nigra=Substantia nigra
Glob Palladus=Globus palladus
Temp Pole=Temporal pole
Cing Gyr=Cingulate gyrus
BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:
AD=Alzheimer's disease brain; patient was demented and showed AD-like pathology upon autopsy
Control=Control brains; patient not demented, showing no neuropathology
Control (Path)=Control brains; pateint not demented but showing sever AD-like pathology
SupTemporal Ctx=Superior Temporal Cortex
Inf Temporal Ctx=Inferior Temporal Cortex

The expression of the gene was analyzed after normalization using scaling factor. The scaling factor is calculated from the Grand mean of CT values for a panel and the Well mean which is specific to the tissue. The Grand mean is the average CT value for all wells across all runs. For example, if a panel has 50 samples and has had 100 probe/primer sets run on it, the grand mean would be the average of these 5000 CT scores. The well mean is tissue-specific. On the above described panel there would be 50 different well means, each taking the average of the 100 CT values generated for each sample on the panel from the 100 probe/primer sets.

The asumption is that across a large number of genes, all samples should have the same CT value. If a well is lower than the average across a large number of genes, it is “scaled up” by that difference or the “scaling factor”.

Scaling Factor=Grand mean−Well mean

The new CT value for the well is:

Scaled CT value=Raw CT+Scaling Factor.

Statistical Analysis of CNS_Neurodegeneration_V1.0 Data

All data were analyzed by analysis of covariance (ANCOVA). As a covariate, the average CT value (or number of rounds of PCR until signal from the well was detected) was calculated for 1000 PCR runs on different genes. This number is therefore an estimate of total cDNA quantity and quality for each sample. When RTQ PCR is run for a given gene, CT values are therefore compared to these average values to correct for differences in well loading or original RNA quality. Stats were run on data from the temporal cortex, as this regions shows sever neurodegeneration in the mid to late stages of the disease, and because the largest number of samples were available for this region giving the most statistical power. Covariates for each well corresponding to Temporal Cortex samples are listed below. The well numbers (10-25) are listed under “Order” in the table of CT values given for each gene run. For this analysis, Controls and Control (Path) cases were grouped together as the intention was to find genes associated with dementia as opposed to amyloid deposition.

Panel CNS_Neurodegeneration_V2.0

| | | |
|---|---|---|
| 10 | AD1 | 33.014 |
| 11 | AD2 | 32.309 |
| 12 | AD3 | 34.195 |
| 13 | AD4 | 32.689 |
| 14 | AD5 Inf | 30.829 |
| 15 | AD5 Sup | 31.519 |
| 16 | AD6 Inf | 31.517 |
| 17 | AD6 Sup | 31.415 |
| 18 | Con1 | 34.236 |
| 19 | Con2 | 32.352 |
| 20 | Con3 | 33.215 |
| 21 | Con4 | 33.661 |
| 22 | Con5 (Path) | 31.685 |
| 23 | Con6 (Path) | 32.187 |
| 24 | Con7 (Path) | 34.427 |
| 25 | Con8 (Path) | 32.238 |

The plates for Panel CNS_Neurodegeneration_V2.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains sixteen brains from Alzheimer's disease (AD) patients, and twenty-nine brains from “Normal controls” who showed no evidence of dementia prior to death. The twenty-nine normal control brains are divided into two categories: Fourteen controls with no dementia and no Alzheimer's like pathology (Controls) and fifteen controls with no dementia but evidence of severe Alzheimer's like pathology, (specifically senile plaque load rated as level 3 on a scale of 0-3; 0=no evidence of plaques, 3=severe AD senile plaque load). Tissue from the temporal cotex (Broddmann Area 21) was selected for all samples from the Harvard Brain Tissue Resource Center; from the two sample from the Human Brain and Spinal Fluid Resource Center (samples 1 and 2) tissue from the inferior and superior temporal cortex was used; each sample on the panel represents a pool of inferior and superior temporal cortex from an individual patient. The temporal cortex was chosen as it shows a loss of neurons in the intermediate stages of the disease. Selection of a region which is affected in the early stages of Alzheimer's disease (e.g., hippocampus or entorhinal cortex) could potentially result in the examination of gene expression after vulnerable neurons are lost, and missing genes involved in the actual neurodegeneration process.

In the labels employed to identify tissues in the CNS_Neurodegeneration_V2.0 panel, the following abbreviations are used:
AD=Alzheimer's disease brain; patient was demented and showed AD-like pathology upon autopsy
Control=Control brains; patient not demented, showing no neuropathology
   AH3=Control brains; pateint not demented but showing sever AD-like pathology
Inf & Sup Temp Ctx Pool=Pool of inferior and superior temporal cortex for a given individual

A. CG50253-01: Epidermal Growth Factor

Expression of full-length physical clone CG50253-01 was assessed using the primer-probe set Ag2505, described in Table AA. Results of the RTQ-PCR runs are shown in Tables AB, AC, AD, AE, AF and AG.

TABLE AA

Probe Name Ag2505

| | | | | |
|---|---|---|---|---|
| | | | Start | SEQ ID |
| Primers | Sequences | Length | Position No | |

| | | | | |
|---|---|---|---|---|
| Forward | 5′-aaagaaggataccagggtgatg-3′ | 22 | 969 | 1213 |
| Probe | TET-5′-atgattgaaccttcaggtccaattca-3′ | 26 | 1020 | 1214 |
| Reverse | 5′-ggtaccatttccctttggtaca-3′ | 22 | 1046 | 1215 |

TABLE AB
AI_comprehensive panel_v1.0

| Tissue Name | A |
|---|---|
| 110967 COPD-F | 15.3 |
| 110980 COPD-F | 11.8 |
| 110968 COPD-M | 8.9 |
| 110977 COPD-M | 28.1 |
| 110989 Emphysema-F | 9.6 |
| 110992 Emphysema-F | 1.9 |
| 110993 Emphysema-F | 7.7 |
| 110994 Emphysema-F | 5.2 |
| 110995 Emphysema-F | 3.6 |
| 110996 Emphysema-F | 0.4 |
| 110997 Asthma-M | 4.6 |
| 111001 Asthma-F | 2.3 |
| 111002 Asthma-F | 3.5 |
| 111003 Atopic Asthma-F | 22.5 |
| 111004 Atopic Asthma-F | 10.4 |
| 111005 Atopic Asthma-F | 7.3 |
| 111006 Atopic Asthma-F | 1.6 |
| 111417 Allergy-M | 5.1 |
| 112347 Allergy-M | 1.4 |
| 112349 Normal Lung-F | 0.7 |
| 112357 Normal Lung-F | 7.1 |
| 112354 Normal Lung-M | 7.6 |
| 112374 Crohns-F | 9.0 |
| 112389 Match Control Crohns-F | 11.2 |
| 112375 Crohns-F | 10.2 |
| 112732 Match Control Crohns-F | 1.2 |
| 112725 Crohns-M | 0.9 |
| 112387 Match Control Crohns-M | 11.4 |
| 112378 Crohns-M | 1.3 |
| 112390 Match Control Crohns-M | 16.7 |
| 112726 Crohns-M | 21.8 |
| 112731 Match Control Crohns-M | 15.3 |
| 112380 Ulcer Col-F | 5.8 |
| 112734 Match Control Ulcer Col-F | 3.7 |
| 112384 Ulcer Col-F | 19.2 |
| 112737 Match Control Ulcer Col-F | 13.5 |
| 112386 Ulcer Col-F | 8.4 |
| 112738 Match Control Ulcer Col-F | 3.8 |
| 112381 Ulcer Col-M | 5.0 |
| 112735 Match Control Ulcer Col-M | 9.7 |
| 112382 Ulcer Col-M | 11.7 |
| 112394 Match Control Ulcer Col-M | 3.1 |
| 112383 Ulcer Col-M | 5.1 |
| 112736 Match Control Ulcer Col-M | 5.0 |
| 112423 Psoriasis-F | 14.0 |
| 112427 Match Control Psoriasis-F | 16.7 |
| 112418 Psoriasis-M | 14.0 |
| 112723 Match Control Psoriasis-M | 0.2 |
| 112419 Psoriasis-M | 18.2 |
| 112424 Match Control Psoriasis-M | 6.8 |
| 112420 Psoriasis-M | 13.9 |
| 112425 Match Control Psoriasis-M | 13.6 |
| 104689 (MF) OA Bone-Backus | 25.3 |
| 104690 (MF) Adj “Normal” Bone-Backus | 27.9 |
| 104691 (MF) OA Synovium-Backus | 2.9 |
| 104692 (BA) OA Cartilage-Backus | 0.0 |
| 104694 (BA) OA Bone-Backus | 5.8 |
| 104695 (BA) Adj “Normal” Bone-Backus | 14.1 |
| 104696 (BA) OA Synovium-Backus | 2.3 |
| 104700 (SS) OA Bone-Backus | 28.9 |
| 104701 (SS) Adj “Normal” Bone-Backus | 25.5 |
| 104702 (SS) OA Synovium-Backus | 11.7 |
| 117093 OA Cartilage Rep7 | 7.5 |
| 112672 OA Bone5 | 19.2 |
| 112673 OA Synovium5 | 6.4 |
| 112674 OA Synovial Fluid cells5 | 6.8 |
| 117100 OA Cartilage Rep14 | 1.9 |
| 112756 OA Bone9 | 26.4 |
| 112757 OA Synovium9 | 2.8 |
| 112758 OA Synovial Fluid Cells9 | 8.1 |
| 117125 RA Cartilage Rep2 | 14.8 |
| 113492 Bone2 RA | 84.7 |
| 113493 Synovium2 RA | 40.9 |
| 113494 Syn Fluid Cells RA | 61.1 |
| 113499 Cartilage4 RA | 90.1 |
| 113500 Bone4 RA | 100.0 |
| 113501 Synovium4 RA | 71.2 |
| 113502 Syn Fluid Cells4 RA | 48.6 |
| 113495 Cartilage3 RA | 77.9 |
| 113496 Bone3 RA | 92.0 |
| 113497 Synovium3 RA | 53.6 |
| 113498 Syn Fluid Cells3 RA | 98.6 |
| 117106 Normal Cartilage Rep20 | 3.0 |
| 113663 Bone3 Normal | 2.0 |
| 113664 Synovium3 Normal | 0.5 |
| 113665 Syn Fluid Cells3 Normal | 1.6 |
| 117107 Normal Cartilage Rep22 | 3.7 |
| 113667 Bone4 Normal | 4.3 |
| 113668 Synovium4 Normal | 9.1 |
| 113669 Syn Fluid Cells4 Normal | 6.6 |

Column A - Rel. Exp. (%) Ag2505, Run 248588456

TABLE AC
CNS_neurodegeneration_v1.0

| Tissue Name | A | B |
|---|---|---|
| AD 1 Hippo | 38.7 | 51.4 |
| AD 2 Hippo | 48.0 | 64.6 |
| AD 3 Hippo | 18.8 | 30.6 |
| AD 4 Hippo | 34.6 | 33.0 |
| AD 5 Hippo | 36.9 | 39.2 |
| AD 6 Hippo | 100.0 | 97.9 |
| Control 2 Hippo | 26.8 | 33.7 |
| Control 4 Hippo | 39.8 | 52.5 |
| Control (Path) 3 Hippo | 18.4 | 21.8 |
| AD 1 Temporal Ctx | 48.6 | 58.6 |
| AD 2 Temporal Ctx | 45.1 | 43.2 |
| AD 3 Temporal Ctx | 37.4 | 33.4 |
| AD 4 Temporal Ctx | 50.3 | 66.4 |
| AD 5 Inf Temporal Ctx | 23.0 | 26.4 |
| AD 5 Sup Temporal Ctx | 42.3 | 43.8 |
| AD 6 Inf Temporal Ctx | 88.3 | 100.0 |
| AD 6 Sup Temporal Ctx | 70.2 | 88.3 |
| Control 1 Temporal Ctx | 16.6 | 16.3 |
| Control 2 Temporal Ctx | 16.7 | 21.6 |
| Control 3 Temporal Ctx | 7.2 | 12.9 |
| Control 3 Temporal Ctx | 19.8 | 43.5 |
| Control (Path) 1 Temporal Ctx | 20.6 | 24.1 |
| Control (Path) 2 Temporal Ctx | 20.6 | 32.1 |
| Control (Path) 3 Temporal Ctx | 14.0 | 17.8 |
| Control (Path) 4 Temporal Ctx | 20.9 | 23.3 |
| AD 1 Occipital Ctx | 31.9 | 35.4 |
| AD 2 Occipital Ctx (Missing) | 5.2 | 4.8 |
| AD 3 Occipital Ctx | 16.7 | 23.5 |
| AD 4 Occipital Ctx | 39.5 | 46.3 |
| AD 5 Occipital Ctx | 24.7 | 22.8 |
| AD 6 Occipital Ctx | 59.9 | 87.7 |
| Control 1 Occipital Ctx | 19.8 | 14.9 |
| Control 2 Occipital Ctx | 26.2 | 29.9 |
| Control 3 Occipital Ctx | 15.2 | 15.7 |
| Control 4 Occipital Ctx | 34.2 | 36.6 |
| Control (Path) 1 Occipital Ctx | 25.5 | 24.1 |
| Control (Path) 2 Occipital Ctx | 15.1 | 20.3 |
| Control (Path) 3 Occipital Ctx | 9.5 | 14.2 |
| Control (Path) 4 Occipital Ctx | 30.6 | 37.6 |
| Control 1 Parietal Ctx | 13.3 | 14.0 |
| Control 2 Parietal Ctx | 46.3 | 57.8 |
| Control 3 Parietal Ctx | 10.1 | 12.9 |
| Control (Path) 1 Parietal Ctx | 24.5 | 29.5 |
| Control (Path) 2 Parietal Ctx | 28.3 | 35.6 |
| Control (Path) 3 Parietal Ctx | 12.7 | 18.9 |
| Control (Path) 4 Parietal Ctx | 28.3 | 32.5 |

Column A - Rel. Exp. (%) Ag2505, Run 208123723
Column B - Rel. Exp. (%) Ag2505, Run 224116291

TABLE AD
Panel 1.3D

| Tissue Name | A |
|---|---|
| Liver adenocarcinoma | 1.8 |
| Pancreas | 13.7 |
| Pancreatic ca. CAPAN 2 | 1.5 |
| Adrenal gland | 3.6 |
| Thyroid | 100.0 |
| Salivary gland | 4.1 |
| Pituitary gland | 37.6 |
| Brain (fetal) | 44.1 |
| Brain (whole) | 9.3 |
| Brain (amygdala) | 8.1 |
| Brain (cerebellum) | 1.8 |
| Brain (hippocampus) | 10.2 |
| Brain (substantia nigra) | 29.3 |
| Brain (thalamus) | 3.6 |
| Cerebral Cortex | 7.7 |
| Spinal cord | 15.2 |
| glio/astro U87-MG | 0.0 |
| glio/astro U-118-MG | 0.0 |
| astrocytoma SW1783 | 0.3 |
| neuro*; met SK-N-AS | 0.4 |
| astrocytoma SF-539 | 1.8 |
| astrocytoma SNB-75 | 2.7 |
| glioma SNB-19 | 0.0 |
| glioma U251 | 9.3 |
| glioma SF-295 | 0.4 |
| Heart (fetal) | 10.0 |
| Heart | 3.1 |
| Skeletal muscle (fetal) | 12.8 |
| Skeletal muscle | 20.9 |
| Bone marrow | 1.2 |
| Thymus | 6.0 |
| Spleen | 6.7 |
| Lymph node | 6.7 |
| Colorectal | 23.5 |
| Stomach | 12.0 |
| Small intestine | 54.3 |
| Colon ca. SW480 | 1.1 |
| Colon ca.* SW620(SW480 met) | 1.4 |
| Colon ca. HT29 | 7.3 |
| Colon ca. HCT-116 | 7.3 |
| Colon ca. CaCo-2 | 10.7 |
| Colon ca. tissue(ODO3866) | 8.5 |
| Colon ca. HCC-2998 | 2.9 |
| Gastric ca.* (liver met) NCI-N87 | 71.7 |
| Bladder | 14.8 |
| Trachea | 21.9 |
| Kidney | 38.2 |
| Kidney (fetal) | 27.5 |
| Renal ca. 786-0 | 0.0 |
| Renal ca. A498 | 0.2 |
| Renal ca. RXF 393 | 39.8 |
| Renal ca. ACHN | 51.1 |
| Renal ca. UO-31 | 0.2 |
| Renal ca. TK-10 | 0.0 |
| Liver | 1.4 |
| Liver (fetal) | 2.3 |
| Liver ca. (hepatoblast) HepG2 | 11.8 |
| Lung | 75.3 |
| Lung (fetal) | 54.7 |
| Lung ca. (small cell) LX-1 | 5.5 |
| Lung ca. (small cell) NCI-H69 | 5.6 |
| Lung ca. (s. cell var.) SHP-77 | 0.2 |
| Lung ca. (large cell)NCI-H460 | 1.2 |
| Lung ca. (non-sm. cell) A549 | 1.2 |
| Lung ca. (non-s. cell) NCI-H23 | 3.2 |
| Lung ca. (non-s. cell) HOP-62 | 0.0 |
| Lung ca. (non-s. cl) NCI-H522 | 0.0 |
| Lung ca. (squam.) SW 900 | 1.8 |
| Lung ca. (squam.) NCI-H596 | 14.6 |
| Mammary gland | 11.9 |
| Breast ca.* (pl. ef) MCF-7 | 89.5 |
| Breast ca.* (pl. ef) MDA-MB-231 | 0.0 |
| Breast ca.* (pl. ef) T47D | 24.7 |
| Breast ca. BT-549 | 2.3 |
| Breast ca. MDA-N | 0.0 |
| Ovary | 3.5 |
| Ovarian ca. OVCAR-3 | 6.4 |
| Ovarian ca. OVCAR-4 | 0.0 |
| Ovarian ca. OVCAR-5 | 0.0 |
| Ovarian ca. OVCAR-8 | 0.2 |
| Ovarian ca. IGROV-1 | 14.5 |
| Ovarian ca.* (ascites) SK-OV-3 | 9.3 |
| Uterus | 27.7 |
| Placenta | 2.9 |
| Prostate | 25.0 |
| Prostate ca.* (bone met)PC-3 | 0.0 |
| Testis | 2.5 |
| Melanoma Hs688(A).T | 0.0 |
| Melanoma* (met) Hs688(B).T | 0.0 |
| Melanoma UACC-62 | 0.0 |
| Melanoma M14 | 0.0 |
| Melanoma LOX IMVI | 0.0 |
| Melanoma* (met) SK-MEL-5 | 0.0 |
| Adipose | 19.3 |

Column A - Rel. Exp. (%) Ag2505, Run 165531061

TABLE AE
Panel 4D

| Tissue Name | A |
|---|---|
| Secondary Th1 act | 0.0 |
| Secondary Th2 act | 0.0 |
| Secondary Tr1 act | 0.2 |
| Secondary Th1 rest | 0.0 |
| Secondary Th2 rest | 0.3 |
| Secondary Tr1 rest | 0.0 |
| Primary Th1 act | 0.0 |
| Primary Th2 act | 0.2 |
| Primary Tr1 act | 0.1 |
| Primary Th1 rest | 0.1 |
| Primary Th2 rest | 0.0 |
| Primary Tr1 rest | 0.1 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.1 |
| PBMC PHA-L | 0.0 |
| Ramos (B cell) none | 0.0 |
| Ramos (B cell) ionomycin | 0.0 |
| B lymphocytes PWM | 0.2 |
| B lymphocytes CD40L and IL-4 | 0.1 |
| EOL-1 dbcAMP | 0.0 |
| EOL-1 dbcAMP PMA/ionomycin | 0.0 |
| Dendritic cells none | 0.1 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.3 |
| HUVEC IL-1beta | 0.2 |
| HUVEC IFN gamma | 0.1 |
| HUVEC TNF alpha + IFN gamma | 0.0 |
| HUVEC TNF alpha + IL4 | 0.1 |
| HUVEC IL-11 | 0.0 |
| Lung Microvascular EC none | 0.0 |
| Lung Microvascular EC TNFalpha + IL-1 beta | 0.0 |
| Microvascular Dermal EC none | 0.0 |
| Microsvasular Dermal EC TNFalpha + IL-1beta | 0.2 |
| Bronchial epithelium TNFalpha + IL1beta | 2.0 |
| Small airway epithelium none | 0.5 |
| Small airway epithelium TNFalpha + IL-1beta | 19.6 |
| Coronery artery SMC rest | 0.0 |
| Coronery artery SMC TNFalpha + IL-1beta | 0.0 |
| Astrocytes rest | 2.0 |
| Astrocytes TNFalpha + IL-1beta | 2.0 |
| KU-812 (Basophil) rest | 0.0 |
| KU-812 (Basophil) PMA/ionomycin | 0.0 |
| CCD1106 (Keratinocytes) none | 0.4 |
| CCD1106 (Keratinocytes) TNFalpha + IL-1beta | 1.3 |
| Liver cirrhosis | 7.5 |
| Lupus kidney | 13.3 |
| NCI-H292 none | 21.8 |
| NCI-H292 IL-4 | 42.0 |
| NCI-H292 IL-9 | 41.8 |
| NCI-H292 IL-13 | 20.9 |
| NCI-H292 IFN gamma | 14.4 |
| HPAEC none | 0.0 |
| HPAEC TNF alpha + IL-1 beta | 0.5 |
| Lung fibroblast none | 4.5 |
| Lung fibroblast TNF alpha + IL-1 beta | 0.3 |
| Lung fibroblast IL-4 | 14.6 |
| Lung fibroblast IL-9 | 3.9 |
| Lung fibroblast IL-13 | 8.7 |
| Lung fibroblast IFN gamma | 14.9 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.2 |
| IBD Colitis 2 | 0.3 |
| IBD Crohn's | 9.9 |
| Colon | 41.2 |
| Lung | 61.6 |
| Thymus | 100.0 |
| Kidney | 21.3 |

Column A - Rel. Exp. (%) Ag2505, Run 164318134

TABLE AF
Panel 5 Islet

| Tissue Name | A |
|---|---|
| 97457_Patient-02go_adipose | 32.3 |
| 97476_Patient-07sk_skeletal muscle | 8.2 |
| 97477_Patient-07ut_uterus | 31.4 |
| 97478_Patient-07pl_placenta | 3.5 |
| 99167_Bayer Patient 1 | 9.5 |
| 97482_Patient-08ut_uterus | 90.1 |
| 97483_Patient-08pl_placenta | 7.4 |
| 97486_Patient-09sk_skeletal muscle | 1.4 |
| 97487_Patient-09ut_uterus | 78.5 |
| 97488_Patient-09pl_placenta | 0.6 |
| 97492_Patient-10ut_uterus | 66.0 |
| 97493_Patient-10pl_placenta | 3.1 |
| 97495_Patient-11go_adipose | 28.3 |
| 97496_Patient-11sk_skeletal muscle | 5.8 |
| 97497_Patient-11ut_uterus | 35.4 |
| 97498_Patient-11pl_placenta | 2.0 |
| 97500_Patient-12go_adipose | 35.1 |
| 97501_Patient-12sk_skeletal muscle | 9.9 |
| 97502_Patient-12ut_uterus | 100.0 |
| 97503_Patient-12pl_placenta | 4.1 |
| 94721_Donor 2 U - A_Mesenchymal Stem Cells | 0.0 |
| 94722_Donor 2 U - B_Mesenchymal Stem Cells | 0.0 |
| 94723_Donor 2 U - C_Mesenchymal Stem Cells | 0.0 |
| 94709_Donor 2 AM - A_adipose | 0.0 |
| 94710_Donor 2 AM - B_adipose | 0.0 |
| 94711_Donor 2 AM - C_adipose | 0.0 |
| 94712_Donor 2 AD - A_adipose | 0.0 |
| 94713_Donor 2 AD - B_adipose | 0.0 |
| 94714_Donor 2 AD - C_adipose | 0.0 |
| 94742_Donor 3 U - A_Mesenchymal Stem Cells | 0.0 |
| 94743_Donor 3 U - B_Mesenchymal Stem Cells | 0.0 |
| 94730_Donor 3 AM - A_adipose | 0.0 |
| 94731_Donor 3 AM - B_adipose | 0.0 |
| 94732_Donor 3 AM - C_adipose | 0.2 |
| 94733_Donor 3 AD - A_adipose | 0.0 |
| 94734_Donor 3 AD - B_adipose | 0.0 |
| 94735_Donor 3 AD - C_adipose | 0.0 |
| 77138_Liver_HepG2untreated | 21.2 |
| 73556_Heart_Cardiac stromal cells (primary) | 0.0 |
| 81735_Small Intestine | 36.9 |
| 72409_Kidney_Proximal Convoluted Tubule | 4.6 |
| 82685_Small intestine_Duodenum | 27.0 |
| 90650_Adrenal_Adrenocortical adenoma | 0.3 |
| 72410_Kidney_HRCE | 7.2 |
| 72411_Kidney_HRE | 10.7 |
| 73139_Uterus_Uterine smooth muscle cells | 0.0 |

Column A - Rel. Exp. (%) Ag2505, Run 248045752

TABLE AG
general oncology screening panel_v_2.4

| Tissue Name | A |
|---|---|
| Colon cancer 1 | 7.7 |
| Colon NAT 1 | 2.5 |
| Colon cancer 2 | 26.4 |
| Colon NAT 2 | 8.4 |
| Colon cancer 3 | 38.2 |
| Colon NAT 3 | 16.7 |
| Colon malignant cancer 4 | 69.7 |
| Colon NAT 4 | 8.7 |
| Lung cancer 1 | 7.2 |
| Lung NAT 1 | 8.0 |
| Lung cancer 2 | 26.1 |
| Lung NAT 2 | 17.4 |
| Squamous cell carcinoma 3 | 19.6 |
| Lung NAT 3 | 8.3 |
| Metastatic melanoma 1 | 14.5 |
| Melanoma 2 | 1.1 |
| Melanoma 3 | 2.5 |
| Metastatic melanoma 4 | 13.5 |
| Metastatic melanoma 5 | 12.6 |
| Bladder cancer 1 | 1.0 |
| Bladder NAT 1 | 0.0 |
| Bladder cancer 2 | 2.4 |
| Bladder NAT 2 | 0.3 |
| Bladder NAT 3 | 0.7 |
| Bladder NAT 4 | 1.5 |
| Prostate adenocarcinoma 1 | 100.0 |
| Prostate adenocarcinoma 2 | 12.9 |
| Prostate adenocarcinoma 3 | 15.1 |
| Prostate adenocarcinoma 4 | 15.9 |
| Prostate NAT 5 | 14.7 |
| Prostate adenocarcinoma 6 | 13.1 |
| Prostate adenocarcinoma 7 | 16.2 |
| Prostate adenocarcinoma 8 | 4.5 |
| Prostate adenocarcinoma 9 | 33.4 |
| Prostate NAT 10 | 7.7 |
| Kidney cancer 1 | 9.6 |
| Kidney NAT 1 | 33.4 |
| Kidney cancer 2 | 19.8 |
| Kidney NAT 2 | 64.6 |
| Kidney cancer 3 | 7.1 |
| Kidney NAT 3 | 29.7 |
| Kidney cancer 4 | 8.5 |
| Kidney NAT 4 | 7.9 |

Column A - Rel. Exp. (%) Ag2505, Run 267145080

AI_comprehensive panel_v1.0 Summary: Ag2505 Highest expression of this gene was seen in a sample derived from rheumatoid arthritis bone (CT=27). This gene displayed ubiquitous expression, but expression of this gene was higher in bone, synovium, cartilage and synovial fluid from RA patients as compared to expression in samples from OA patients, normal and diseased lung samples. Expression of this gene was downregulated in Crohn's samples as compared to the corresponding control samples. This gene encodes a putative novel adhesion molecule which is homologous to mouse POEM (preosteoblast epidermal growth factor-like repeat protein with meprin)or nephronectin. Murine nephronectin may function in multiple biological processes including development of the kidney (Miner J H. J Cell Biol 2001 Jul 23; 154(2):257-9) and bone and contribute to liver and lung fibrosis (Levine et al., 2000, Am J Pathol 2000 June; 156(6):1927-35). Therapeutic modulation of this gene or gene product is useful in the treatment of autoimmune and inflammatory diseases such as rheumatoid and osteoarthritis, Inflammatory bowel disease, COPD, asthma, psoriasis, liver and lung fibrosis.

CNS_neurodegeneration_v1.0 Summary: Ag2505 Greater expression of this gene in the temporal cortex of Alzheimer's disease patients demonstrates a functional role for this gene, an EGF homolog, in neurodegenerative disease. Alpha secretase activity, which is generally believed to be a beneficial processing alternative to beta secretase, is increased by EGF in neuronal cells. (Slack B E. C.Biochem J 1997 October 1;327 ( Pt 1):245-9) This suggests the increased expression observed here is a compensatory action in the brain to counter the mechanisms of Alzheimer's Disease. This gene or the protein encoded by this gene is a potential therapeutic agent for the treatment of Alzheimer's disease and other neurodegenerative diseases.

EGF is also known to facilitate long term potentiation (LTP) in the hippocampus, a process thought to underlie learning and memory. (Abe K Brain Res 1992 Octobter 16;593(2):335-8 ) This gene has utility in treating disorders of memory, such as neurodegenerative diseases and aging, when used alone or incombination with other growth factors such as bFGF.

In addition, EGF supports the growth and differentiation of dopaniinergic neurons, which are selectively vulnerable to loss in Parkinson's disease. (Storch A. Exp Neurol 2001 August; 170(2):317-25 ). This gene or gene product has utility in treating Parkinson's Disease.

Panel 1.3D Summary: Ag2505 Highest expression of this gene was detected in the thyroid (CT=29.3), with lower but still significant levels of expression seen in other metabolic tissues, including skeletal muscle, fetal skeletal muscle, small intestine, stomach, pancreas, adipose and fetal heart. Very low levels were also seen in heart and placenta. This gene encodes a putative novel adhesion molecule. Studies in mouse have revealed a gene (perhaps the mouse ortholog of this human gene) very homologous to this gene that is called POEM (preosteoblast epidermal growth factor-like repeat protein with meprin, A5 protein, and receptor protein-tyrosine phosphatase mu domain; Morimura N et al., J Biol Chem 2001 November 9;276(45):42172-81) or nephronectin (Brandenberger R et al., J Cell Biol 2001 July 23;154(2):447-58). POEM/nephronectin seems to be a ligand for the alpha8betal integrin as evidenced by two independent sets of published data. Integrins are known to mediate development and organogenesis. (Schwartz et al., Annu. Rev. Cell Dev. Biol. 11, 549-599, 1995; Clark and Brugge, Science 268, 233-239, 1995)Other known ligands for the alpha8beta1 integrin include fibronectin, vitronectin, tenascin, and osteopontin. Modulation of the expression or activity of this gene or gene product by protein, small molecule, or antibody therapeutics is an effective therapeutic for disorders involving alpha8beta1 integrin signaling.

Overall, this gene is expressed at a low to moderate level in the normal tissues on this panel. The brain, prostate, lung and colon cancer cell lines show a very low level of expression compared to the normal organs. This molecule is a candidate as a therapeutic inhibitor for these cancers.

Expression in the brain in the substantia nigra, hippocampus, cortex, amygdala, thalamus and spinal cord indicates a further functional role for this gene product in CNS processes mediated by these regions. Please see CNS_neurodegeneration_v1.0 for discussion of utility in the central nervous system.

Panel 4D Summary: Ag 2505 Highest expression of this transcript was found in the thymus and the lung (CTs=27-28). Consistent with this lung expression, this transcript was found in the pulmonary mucoepidermoid cell line H292 and was up-regulated upon treatment with the Th2 cytokines IL4 and IL9. This gene was also expressed at lower levels in lung fibroblasts treated with IL4. Modulation of the expression or activity of this gene or gene product by small molecule, protein or antibody therapeutics is beneficial for the treatment of inflammatory lung diseases such as asthma, emphysema and chronic obstructive pulmonary diseases. Therapeutics designed with the gene or the protein encoded for by this transcript are important for maintaining or restoring normal function of thymus during inflammation.

Panel 5 Islet Summary: Ag2505 Highest expression of this gene was detected in uterus (CT=30). Moderate expression of this gene was also seen in adipose and skeletal muscle of gestational diabetic patients both requiring and not requiring daily injections of insulin. This gene was also expressed in samples derived from a pregnant and a nondiabetic, but overweight patient. In addition, this gene was also expressed in islet beta cells (those that are insulin producing) and small intestine. Therefore, therapeutic modulation of this gene or gene product is useful in the treatment of metabolically related diseases including obesity, Type I and Type II diabetes.

general oncology screening panel_v_{—}2.4 Summary: Ag2505 Highest expression of this gene was detected in prostate cancer (CT=27.7). Moderate to low levels of expression of this gene was seen in both normal and cancer samples derived from colon, lung, prostate and kidney. Consistent with panels 2.2 and 2D, expression of this gene was downregulated in kidney cancer as compared to normal kidney. But higher expression of this gene was seen in colon cancer as compared to corresponding normal adjacent sample. Therefore, expression of this gene distinguishes between cancer and normal kidney and colon tissue. Please see panel 1.3, 1.6, 2.2 for further discussion on the utility of this gene.

B. CG50377-01 and CG50377-02 and CG50377-03 and CG50377-04 and CG50377-05 and CG50377-06: (Cub-Sushi)x Domain Containing Protein

Expression of gene CG50377-01 and variants CG50377-02, CG50377-03, CG50377-04, CG50377-05 and CG50377-06 was assessed using the primer-probe sets Ag2420 and Ag5824, described in Tables BA and BB. Results of the RTQ-PCR runs are shown in Tables BC, BD, BE and BF.

TABLE BA

Probe Name Ag2420

| | | | | |
|---|---|---|---|---|
| | | | Start | SEQ ID |
| Primers | Sequences | Length | Position | No |

| | | | | |
|---|---|---|---|---|
| Forward | 5′-ctgcacttggctggaactta-3′ | 20 | 9465 | 1216 |
| Probe | TET-5′-tttcatctcctactccaggtgtacca-3′- | 26 | 9498 | 1217 |
| | TAMRA | | | |
| Reverse | 5′-atctccacaggccctgtaat-3′ | 20 | 9525 | 1218 |

TABLE BB
Probe Name Ag5824

| | | | | |
|---|---|---|---|---|
| | | | Start | SEQ ID |
| Primers | Sequences | Length | Position | No |

| | | | | |
|---|---|---|---|---|
| Forward | 5′-ccgcttcatctctgatttctc-3′ | 21 | 2319 | 1219 |
| Probe | TET-5′-tgaaggattcaacatcaccttctcag-3′- | 26 | 2349 | 1220 |
| | TAMRA | | | |
| Reverse | 5′-acagggctccaagtcgtact-3′ | 20 | 2375 | 1221 |

TABLE BC
General_screening_panel_v1.5

| Tissue Name | A |
|---|---|
| Adipose | 0.0 |
| Melanoma* Hs688(A).T | 6.7 |
| Melanoma* Hs688(B).T | 4.6 |
| Melanoma* M14 | 0.0 |
| Melanoma* LOXIMVI | 0.0 |
| Melanoma* SK-MEL-5 | 0.0 |
| Squamous cell carcinoma SCC-4 | 0.3 |
| Testis Pool | 0.5 |
| Prostate ca.* (bone met) PC-3 | 0.0 |
| Prostate Pool | 0.1 |
| Placenta | 0.0 |
| Uterus Pool | 0.1 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 0.0 |
| Ovarian ca. IGROV-1 | 0.2 |
| Ovarian ca. OVCAR-8 | 0.8 |
| Ovary | 0.1 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 0.2 |
| Trachea | 0.3 |
| Lung | 0.0 |
| Fetal Lung | 0.0 |
| Lung ca. NCI-N417 | 0.0 |
| Lung ca. LX-1 | 0.0 |
| Lung ca. NCI-H146 | 0.0 |
| Lung ca. SHP-77 | 0.0 |
| Lung ca. A549 | 0.0 |
| Lung ca. NCI-H526 | 0.5 |
| Lung ca. NCI-H23 | 0.3 |
| Lung ca. NCI-H460 | 0.0 |
| Lung ca. HOP-62 | 0.1 |
| Lung ca. NCI-H522 | 0.8 |
| Liver | 0.0 |
| Fetal Liver | 0.1 |
| Liver ca. HepG2 | 0.0 |
| Kidney Pool | 0.1 |
| Fetal Kidney | 0.9 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 1.6 |
| Gastric ca. (liver met.) NCI-N87 | 0.0 |
| Gastric ca. KATO III | 0.0 |
| Colon ca. SW-948 | 0.0 |
| Colon ca. SW480 | 0.2 |
| Colon ca.* (SW480 met) SW620 | 0.0 |
| Colon ca. HT29 | 0.0 |
| Colon ca. HCT-116 | 0.0 |
| Colon ca. CaCo-2 | 0.1 |
| Colon cancer tissue | 1.0 |
| Colon ca. SW1116 | 0.0 |
| Colon ca. Colo-205 | 0.0 |
| Colon ca. SW-48 | 0.0 |
| Colon Pool | 0.1 |
| Small Intestine Pool | 0.6 |
| Stomach Pool | 0.3 |
| Bone Marrow Pool | 0.1 |
| Fetal Heart | 0.1 |
| Heart Pool | 0.1 |
| Lymph Node Pool | 0.2 |
| Fetal Skeletal Muscle | 0.3 |
| Skeletal Muscle Pool | 0.1 |
| Spleen Pool | 0.7 |
| Thymus Pool | 0.5 |
| CNS cancer (glio/astro) U87-MG | 100.0 |
| CNS cancer (glio/astro) U-118-MG | 4.6 |
| CNS cancer (neuro; met) SK-N-AS | 0.0 |
| CNS cancer (astro) SF-539 | 0.0 |
| CNS cancer (astro) SNB-75 | 1.0 |
| CNS cancer (glio) SNB-19 | 0.6 |
| CNS cancer (glio) SF-295 | 19.6 |
| Brain (Amygdala) Pool | 4.0 |
| Brain (cerebellum) | 9.5 |
| Brain (fetal) | 86.5 |
| Brain (Hippocampus) Pool | 5.4 |
| Cerebral Cortex Pool | 5.8 |
| Brain (Substantia nigra) Pool | 4.9 |
| Brain (Thalamus) Pool | 7.7 |
| Brain (whole) | 7.5 |
| Spinal Cord Pool | 3.3 |
| Adrenal Gland | 1.4 |
| Pituitary gland Pool | 0.2 |
| Salivary Gland | 0.0 |
| Thyroid (female) | 0.0 |
| Pancreatic ca. CAPAN2 | 0.0 |
| Pancreas Pool | 0.3 |

Column A - Rel. Exp. (%) Ag5824, Run 246273272

TABLE BD
Panel 1.3D

| Tissue Name | A |
|---|---|
| Liver adenocarcinoma | 0.0 |
| Pancreas | 0.9 |
| Pancreatic ca. CAPAN 2 | 0.0 |
| Adrenal gland | 1.1 |
| Thyroid | 0.2 |
| Salivary gland | 0.0 |
| Pituitary gland | 0.4 |
| Brain (fetal) | 100.0 |
| Brain (whole) | 13.6 |
| Brain (amygdala) | 27.4 |
| Brain (cerebellum) | 3.9 |
| Brain (hippocampus) | 95.3 |
| Brain (substantia nigra) | 5.0 |
| Brain (thalamus) | 7.9 |
| Cerebral Cortex | 37.6 |
| Spinal cord | 2.5 |
| glio/astro U87-MG | 50.3 |
| glio/astro U-118-MG | 2.7 |
| astrocytoma SW1783 | 10.9 |
| neuro*; met SK-N-AS | 0.0 |
| astrocytoma SF-539 | 0.0 |
| astrocytoma SNB-75 | 0.0 |
| glioma SNB-19 | 0.8 |
| glioma U251 | 1.4 |
| glioma SF-295 | 7.8 |
| Heart (fetal) | 0.0 |
| Heart | 0.2 |
| Skeletal muscle (fetal) | 1.3 |
| Skeletal muscle | 0.0 |
| Bone marrow | 0.0 |
| Thymus | 0.0 |
| Spleen | 1.2 |
| Lymph node | 0.2 |
| Colorectal | 0.0 |
| Stomach | 0.2 |
| Small intestine | 1.7 |
| Colon ca. SW480 | 0.3 |
| Colon ca.* SW620(SW480 met) | 0.0 |
| Colon ca. HT29 | 0.0 |
| Colon ca. HCT-116 | 0.0 |
| Colon ca. CaCo-2 | 0.0 |
| Colon ca. tissue(ODO3866) | 1.1 |
| Colon ca. HCC-2998 | 0.0 |
| Gastric ca.* (liver met) NCI-N87 | 0.0 |
| Bladder | 0.6 |
| Trachea | 0.2 |
| Kidney | 0.0 |
| Kidney (fetal) | 0.6 |
| Renal ca. 786-0 | 0.0 |
| Renal ca. A498 | 0.2 |
| Renal ca. RXF 393 | 0.0 |
| Renal ca. ACHN | 0.0 |
| Renal ca. UO-31 | 0.0 |
| Renal ca. TK-10 | 0.0 |
| Liver | 0.0 |
| Liver (fetal) | 0.2 |
| Liver ca. (hepatoblast) HepG2 | 0.0 |
| Lung | 0.6 |
| Lung (fetal) | 0.2 |
| Lung ca. (small cell) LX-1 | 0.0 |
| Lung ca. (small cell) NCI-H69 | 0.0 |
| Lung ca. (s. cell var.) SHP-77 | 0.0 |
| Lung ca. (large cell)NCI-H460 | 0.0 |
| Lung ca. (non-sm. cell) A549 | 0.0 |
| Lung ca. (non-s. cell) NCI-H23 | 0.0 |
| Lung ca. (non-s. cell) HOP-62 | 0.0 |
| Lung ca. (non-s. cl) NCI-H522 | 0.2 |
| Lung ca. (squam.) SW 900 | 0.0 |
| Lung ca. (squam.) NCI-H596 | 0.0 |
| Mammary gland | 0.4 |
| Breast ca.* (pl. ef) MCF-7 | 0.0 |
| Breast ca.* (pl. ef) MDA-MB-231 | 0.0 |
| Breast ca.* (pl. ef) T47D | 0.0 |
| Breast ca. BT-549 | 1.3 |
| Breast ca. MDA-N | 0.0 |
| Ovary | 0.5 |
| Ovarian ca. OVCAR-3 | 0.0 |
| Ovarian ca. OVCAR-4 | 0.0 |
| Ovarian ca. OVCAR-5 | 0.0 |
| Ovarian ca. OVCAR-8 | 0.0 |
| Ovarian ca. IGROV-1 | 0.0 |
| Ovarian ca.* (ascites) SK-OV-3 | 0.0 |
| Uterus | 0.0 |
| Placenta | 0.2 |
| Prostate | 0.4 |
| Prostate ca.* (bone met)PC-3 | 0.0 |
| Testis | 1.3 |
| Melanoma Hs688(A).T | 2.4 |
| Melanoma* (met) Hs688(B).T | 0.3 |
| Melanoma UACC-62 | 0.0 |
| Melanoma M14 | 0.0 |
| Melanoma LOX IMVI | 0.0 |
| Melanoma* (met) SK-MEL-5 | 0.0 |
| Adipose | 0.5 |

Column A - Rel. Exp. (%) Ag2420, Run 159253002

TABLE BE
Panel 4.1D

| Tissue Name | A |
|---|---|
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 10.7 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.7 |
| Small airway epithelium none | 1.3 |
| Small airway epithelium TNFalpha + IL-1beta | 0.0 |
| Coronery artery SMC rest | 2.4 |
| Coronery artery SMC TNFalpha + IL-1beta | 4.5 |
| Astrocytes rest | 15.7 |
| Astrocytes TNFalpha + IL-1beta | 20.6 |
| KU-812 (Basophil) rest | 0.0 |
| KU-812 (Basophil) PMA/ionomycin | 0.0 |
| CCD1106 (Keratinocytes) none | 3.6 |
| CCD1106 (Keratinocytes) TNFalpha + IL-1beta | 3.9 |
| Liver cirrhosis | 1.6 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 0.0 |
| HPAEC TNF alpha + IL-1 beta | 0.0 |
| Lung fibroblast none | 75.8 |
| Lung fibroblast TNF alpha + IL-1 beta | 5.4 |
| Lung fibroblast IL-4 | 28.9 |
| Lung fibroblast IL-9 | 100.0 |
| Lung fibroblast IL-13 | 2.9 |
| Lung fibroblast IFN gamma | 45.7 |
| Dermal fibroblast CCD1070 rest | 52.1 |
| Dermal fibroblast CCD1070 TNF alpha | 39.0 |
| Dermal fibroblast CCD1070 IL-1 beta | 47.3 |
| Dermal fibroblast IFN gamma | 4.6 |
| Dermal fibroblast IL-4 | 11.8 |
| Dermal Fibroblasts rest | 26.4 |
| Neutrophils TNFa + LPS | 0.0 |
| Neutrophils rest | 0.0 |
| Colon | 0.0 |
| Lung | 0.8 |
| Thymus | 0.0 |
| Kidney | 0.0 |

Column A - Rel. Exp. (%) Ag5824, Run 246921321

TABLE BF
Panel 4D

| Tissue Name | A |
|---|---|
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 11.3 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 1.0 |
| Small airway epithelium TNFalpha + IL-1beta | 0.0 |
| Coronery artery SMC rest | 3.9 |
| Coronery artery SMC TNFalpha + IL-1 beta | 3.2 |
| Astrocytes rest | 100.0 |
| Astrocytes TNFalpha + IL-1beta | 32.8 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL-1beta | 0.0 |
| Liver cirrhosis | 0.0 |
| Lupus kidney | 0.0 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 0.0 |
| HPAEC TNF alpha + IL-1 beta | 0.0 |
| Lung fibroblast none | 36.3 |
| Lung fibroblast TNF alpha + IL-1 beta | 4.6 |
| Lung fibroblast IL-4 | 52.9 |
| Lung fibroblast IL-9 | 78.5 |
| Lung fibroblast IL-13 | 37.4 |
| Lung fibroblast IFN gamma | 37.6 |
| Dermal fibroblast CCD1070 rest | 63.3 |
| Dermal fibroblast CCD1070 TNF alpha | 27.0 |
| Dermal fibroblast CCD1070 IL-1 beta | 20.3 |
| Dermal fibroblast IFN gamma | 8.6 |
| Dermal fibroblast IL-4 | 4.2 |
| IBD Colitis 2 | 0.0 |
| IBD Crohn's | 0.0 |
| Colon | 0.7 |
| Lung | 0.8 |
| Thymus | 1.9 |
| Kidney | 0.0 |

Column A - Rel. Exp. (%) Ag2420, Run 159255381

General_screeningpanel_v1.5 Summary: Ag5824 Highest expression of this gene was detected in a brain cancer cell line (CT=28.2). Significant expression of this gene was also seen in two melanoma and three brain cancer cell lines. Therefore, expression of this gene detects the presence of melanoma and brain cancer. Therapeutic modulation of this gene or its protein product is useful in the treatment of these cancers.

In addition, this gene was moderately expressed in all the regions of central nervous system examined, including amygdala, hippocampus, substantia nigra, thalamus, cerebellum, cerebral cortex, and spinal cord. Therapeutic modulation of this gene or gene product is useful in the treatment of central nervous system disorders such as Alzheimer's disease, Parkinson's disease, epilepsy, multiple sclerosis, schizophrenia and depression.

Panel 1.1 Summary: Ag575 Expression of this gene was restricted to normal brain derived tissue. In addition, expression was associated with a number of samples derived from brain cancer cell lines, with highest expression seen in the brain cancer cell line U87-MG (CT=23.5). Thus, the expression of this gene distinguishes these brain derived tissues from other samples in the panel. Therapeutic modulation of this gene or gene product, through the use of antibodies, small molecule drugs or protein therapeutics is of benefit in the treatment of brain cancer.

This gene also had moderate levels of expression in a number of metabolic tissues including adrenal, pituitary, heart, and fetal skeletal muscle.

Panel 1.3D Summary: Ag2420 Expression of this gene was restricted to normal brain derived tissue, with highest expression seen in the fetal brain (CT=29.8). In addition, expression was associated with a number of samples derived from brain cancer cell lines. Thus, the expression of this gene distinguishes these brain derived tissues from other samples in the panel. Therapeutic modulation of this gene or gene product, through the use of antibodies, small molecule drugs or protein therapeutics is of benefit in the treatment of brain cancer.

This gene represents a novel protein containing CUB and sushi domains. Its expression profile was highly brain-preferential; levels in the CNS appear 10-fold greater than in other tissues. At least one brain-specific protein containing CUB and sushi domains has been linked to seizures, and shows differential expression in response to pentylentetrazole (Shimizu-Nishikawa K, Brain Res Mol Brain Res 1995 February; 28(2):201-10). This gene or its protein product is a drug target for the treatment of epilepsy or any seizure disorder.

Panel 4.1D Summary: Ag5824 Highest expression of this gene was detected in IL-9 activated lung fibroblast cells (CT=32). Significant expression of this gene was also seen in resting and activated astrocytes, lung fibroblasts and dermal fibroblasts. The expression pattern in this panel was in agreement with that seen in panel 4D. Please see panel 4D for further discussion on the utility of this gene.

Panel 4D Summary: Ag2420 This gene encodes a cub-domain and sushi-domain containing single-pass membrane protein. Highest expression of this gene was seen in resting astrocytes (CT=30.6). Significant expression of this gene was also detected in TNF-alpha+IL-1-beta-stimulated astrocytes, resting and cytokine-stimulated lung fibroblasts and dermal fibroblasts. Therefore, an isolated extracellular domain of this gene or its protein product is useful as a therapeutic protein to reduce or eliminate the symptoms of multiple sclerosis, chronic obstructive pulmonary disease, asthma, or emphysema, and psoriasis.

Agonist or antagonist antibodies that stimulate or inhibit the function of this gene or its protein product are useful as therapeutics to reduce or eliminate the symptoms of multiple sclerosis, chronic obstructive pulmonary disease, asthma, or emphysema, and psoriasis.

C. CG5038904: Interleukin-1 Receptor Related Protein Like

Expression of gene CG50389-04 was assessed using the primer-probe set Ag48 11, described in Table CA. Results of the RTQ-PCR runs are shown in Tables CB and CC.

TABLE CA
Probe Name Ag4811

| | | | | |
|---|---|---|---|---|
| | | | Start | SEQ ID |
| Primers | Sequences | Length | Position | No |

| | | | | |
|---|---|---|---|---|
| Forward | 5′-catgtctcttttcgggaacata-3′ | 22 | 918 | 1222 |
| Probe | TET-5′-cacagtaaacatcaccttcttggaagtg-3′- | 28 | 947 | 1223 |
| | TAMRA | | | |
| Reverse | 5′-gaaggccataatcttccatttt-3′ | 22 | 975 | 1224 |

TABLE CB
General_screening_panel_v1.4

| Tissue Name | A |
|---|---|
| Adipose | 4.9 |
| Melanoma* Hs688(A).T | 1.4 |
| Melanoma* Hs688(B).T | 2.9 |
| Melanoma* M14 | 0.0 |
| Melanoma* LOXIMVI | 0.0 |
| Melanoma* SK-MEL-5 | 0.0 |
| Squamous cell carcinoma SCC-4 | 45.4 |
| Testis Pool | 1.7 |
| Prostate ca.* (bone met) PC-3 | 0.0 |
| Prostate Pool | 2.7 |
| Placenta | 0.5 |
| Uterus Pool | 0.4 |
| Ovarian ca. OVCAR-3 | 1.4 |
| Ovarian ca. SK-OV-3 | 0.3 |
| Ovarian ca. OVCAR-4 | 45.1 |
| Ovarian ca. OVCAR-5 | 0.0 |
| Ovarian ca. IGROV-1 | 0.4 |
| Ovarian ca. OVCAR-8 | 25.9 |
| Ovary | 6.0 |
| Breast ca. MCF-7 | 0.0 |
| Breast ca. MDA-MB-231 | 32.1 |
| Breast ca. BT 549 | 10.9 |
| Breast ca. T47D | 1.2 |
| Breast ca. MDA-N | 0.0 |
| Breast Pool | 6.3 |
| Trachea | 8.1 |
| Lung | 0.1 |
| Fetal Lung | 5.3 |
| Lung ca. NCI-N417 | 0.0 |
| Lung ca. LX-1 | 3.9 |
| Lung ca. NCI-H146 | 0.0 |
| Lung ca. SHP-77 | 0.0 |
| Lung ca. A549 | 0.1 |
| Lung ca. NCI-H526 | 0.0 |
| Lung ca. NCI-H23 | 3.8 |
| Lung ca. NCI-H460 | 0.0 |
| Lung ca. HOP-62 | 0.1 |
| Lung ca. NCI-H522 | 0.0 |
| Liver | 1.2 |
| Fetal Liver | 4.3 |
| Liver ca. HepG2 | 0.0 |
| Kidney Pool | 3.2 |
| Fetal Kidney | 5.1 |
| Renal ca. 786-0 | 0.0 |
| Renal ca. A498 | 0.0 |
| Renal ca. ACHN | 0.0 |
| Renal ca. UO-31 | 5.3 |
| Renal ca. TK-10 | 71.7 |
| Bladder | 6.5 |
| Gastric ca. (liver met.) NCI-N87 | 100.0 |
| Gastric ca. KATO III | 24.1 |
| Colon ca. SW-948 | 2.4 |
| Colon ca. SW480 | 7.1 |
| Colon ca.* (SW480 met) SW620 | 0.5 |
| Colon ca. HT29 | 16.8 |
| Colon ca. HCT-116 | 0.0 |
| Colon ca. CaCo-2 | 0.5 |
| Colon cancer tissue | 12.1 |
| Colon ca. SW1116 | 6.9 |
| Colon ca. Colo-205 | 3.7 |
| Colon ca. SW-48 | 0.0 |
| Colon Pool | 1.6 |
| Small Intestine Pool | 1.2 |
| Stomach Pool | 7.0 |
| Bone Marrow Pool | 3.1 |
| Fetal Heart | 0.4 |
| Heart Pool | 1.3 |
| Lymph Node Pool | 2.8 |
| Fetal Skeletal Muscle | 0.6 |
| Skeletal Muscle Pool | 3.0 |
| Spleen Pool | 4.2 |
| Thymus Pool | 3.8 |
| CNS cancer (glio/astro) U87-MG | 0.2 |
| CNS cancer (glio/astro) U-118-MG | 0.8 |
| CNS cancer (neuro; met) SK-N-AS | 0.3 |
| CNS cancer (astro) SF-539 | 0.4 |
| CNS cancer (astro) SNB-75 | 4.9 |
| CNS cancer (glio) SNB-19 | 0.0 |
| CNS cancer (glio) SF-295 | 0.0 |
| Brain (Amygdala) Pool | 4.6 |
| Brain (cerebellum) | 0.4 |
| Brain (fetal) | 4.7 |
| Brain (Hippocampus) Pool | 3.0 |
| Cerebral Cortex Pool | 6.9 |
| Brain (Substantia nigra) Pool | 4.9 |
| Brain (Thalamus) Pool | 9.2 |
| Brain (whole) | 7.0 |
| Spinal Cord Pool | 0.8 |
| Adrenal Gland | 12.5 |
| Pituitary gland Pool | 0.1 |
| Salivary Gland | 2.8 |
| Thyroid (female) | 6.3 |
| Pancreatic ca. CAPAN2 | 0.0 |
| Pancreas Pool | 21.0 |

Column A - Rel. Exp. (%) Ag4811, Run 223204488

TABLE CC
Panel 4.1D

| Tissue Name | A |
|---|---|
| Secondary Th1 act | 0.0 |
| Secondary Th2 act | 0.0 |
| Secondary Tr1 act | 1.5 |
| Secondary Th1 rest | 0.0 |
| Secondary Th2 rest | 0.0 |
| Secondary Tr1 rest | 0.0 |
| Primary Th1 act | 0.9 |
| Primary Th2 act | 5.1 |
| Primary Tr1 act | 2.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 3.3 |
| CD8 lymphocyte act | 0.7 |
| Secondary CD8 lymphocyte rest | 2.9 |
| Secondary CD8 lymphocyte act | 0.7 |
| CD4 lymphocyte none | 0.0 |
| 2ry Th1/Th2/Tr1_anti-CD95 CH11 | 0.0 |
| LAK cells rest | 3.9 |
| LAK cells IL-2 | 0.0 |
| LAK cells IL-2 + IL-12 | 0.0 |
| LAK cells IL-2 + IFN gamma | 1.2 |
| LAK cells IL-2 + IL-18 | 0.7 |
| LAK cells PMA/ionomycin | 11.1 |
| NK Cells IL-2 rest | 0.0 |
| Two Way MLR 3 day | 0.0 |
| Two Way MLR 5 day | 0.7 |
| Two Way MLR 7 day | 1.6 |
| PBMC rest | 0.0 |
| PBMC PWM | 4.8 |
| PBMC PHA-L | 0.0 |
| Ramos (B cell) none | 0.0 |
| Ramos (B cell) ionomycin | 0.0 |
| B lymphocytes PWM | 10.7 |
| B lymphocytes CD40L and IL-4 | 0.8 |
| EOL-1 dbcAMP | 0.0 |
| EOL-1 dbcAMP PMA/ionomycin | 2.7 |
| Dendritic cells none | 9.4 |
| Dendritic cells LPS | 0.6 |
| Dendritic cells anti-CD40 | 15.5 |
| Monocytes rest | 0.0 |
| Monocytes LPS | 8.8 |
| Macrophages rest | 1.6 |
| Macrophages LPS | 0.0 |
| HUVEC none | 0.0 |
| HUVEC starved | 0.0 |
| HUVEC IL-1beta | 1.7 |
| HUVEC IFN gamma | 2.3 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 8.5 |
| Small airway epithelium none | 11.1 |
| Small airway epithelium TNFalpha + IL-1beta | 14.9 |
| Coronery artery SMC rest | 3.1 |
| Coronery artery SMC TNFalpha + IL-1beta | 1.4 |
| Astrocytes rest | 2.2 |
| Astrocytes TNFalpha + IL-1beta | 0.0 |
| KU-812 (Basophil) rest | 0.0 |
| KU-812 (Basophil) PMA/ionomycin | 0.0 |
| CCD1106 (Keratinocytes) none | 100.0 |
| CCD1106 (Keratinocytes) TNFalpha + IL-1beta | 89.5 |
| Liver cirrhosis | 4.6 |
| NCI-H292 none | 2.4 |
| NCI-H292 IL-4 | 5.0 |
| NCI-H292 IL-9 | 1.9 |
| NCI-H292 IL-13 | 6.0 |
| NCI-H292 IFN gamma | 3.9 |
| HPAEC none | 2.4 |
| HPAEC TNF alpha + IL-1 beta | 0.0 |
| Lung fibroblast none | 26.1 |
| Lung fibroblast TNF alpha + IL-1 beta | 17.1 |
| Lung fibroblast IL-4 | 33.9 |
| Lung fibroblast IL-9 | 27.7 |
| Lung fibroblast IL-13 | 25.9 |
| Lung fibroblast IFN gamma | 52.1 |
| Dermal fibroblast CCD1070 rest | 9.8 |
| Dermal fibroblast CCD1070 TNF alpha | 5.4 |
| Dermal fibroblast CCD1070 IL-1 beta | 4.3 |
| Dermal fibroblast IFN gamma | 5.6 |
| Dermal fibroblast IL-4 | 14.9 |
| Dermal Fibroblasts rest | 7.5 |
| Neutrophils TNFa + LPS | 54.7 |
| Neutrophils rest | 0.9 |
| Colon | 0.8 |
| Lung | 4.4 |
| Thymus | 4.5 |
| Kidney | 29.9 |

Column A - Rel. Exp. (%) Ag4811, Run 223273603

General_screeningpanel_v1.4 Summary: Ag4811 The highest expression of this gene was detected in a sample derived from a gastric cancer cell line (NCI-N87) (CT=29.2). There was low to moderate expression associated with other gastric cancer cell lines, colon cancer cell lines, breast cancer cell lines, ovarian cancer cell lines and a renal cancer cell line. The expression of this gene or protein product is useful as a marker for gastric cancer, colon cancer, breast cancer, ovarian cancer or renal cancer. Therapeutic modulation of this gene or its protein product through the use of antibodies, protein therapeutics or small molecule drugs is useful in the treatment of gastric cancer, colon cancer, breast cancer, ovarian cancer or renal cancer.

This gene was also moderately expressed in a number of metabolic tissues including adipose, fetal liver, skeletal muscle, adrenal, thyroid and pancreas. Therapeutic modulation of this gene, expressed protein and/or use of antibodies, protein therapeutics or small molecule drugs targeting the gene or gene product are useful in the treatment of metabolic diseases, including obesity and Types 1 and 2 diabetes.

This gene product was expressed at low levels in the brain. This gene encodes a protein with homology to an interleukin 1 receptor-like protein. Interleukins are involved in many brain pathologies due to the pro-inflammatory action of interleukins. Inflammation is a key pathological process mediating the damage seen in many disease states including stroke, Alzheimer's disease, and spinocerebellar ataxias (J Neurosci 2001 August 1;21(15):5389-96). IL-1 receptor like protein has been shown to bind IL-18 (J Immunol 1999 May 1;162(9):5041-4), which is involved in CNS inflammation. Moreover, anti-inflammatory agents that are associated with reduced risk of Alzheimer's Disease, such as NSAIDS (Drug Saf 2001;24(11):801-11), have been shown to inhibit IL-18 expression. Therapeutic modulation of this gene, expressed protein and/or use of antibodies or small molecule drugs targeting the gene or gene product are useful in the treatment of stroke, Alzheimer's disease, and spinocerebellar ataxias and any other CNS disorder in which inflammation plays a role.

Panel 4.1D Summary: Ag 4811 The highest expression of this gene was detected in keratinocytes, regardless of their treatment. This transcript encodes a novel IL-1 receptor related protein that may have the potential to trigger novel members of the IL-1 family members as described by Debets et. al. (J Immunol 2001 August 1;167(3):1440-6; J Biol Chem 1996 February 23;271(8):3967-70). Novel IL1 receptor like molecules have been cloned and reported to lead to activation of NF-KB and IL18 production, a potent inflammatory cytokine associated with lung inflammation, IBD and psoriasis. IL1 R family members have also been shown to mediate inflammatory signals through NF-kB activation, among other signalling pathways. Therapeutic modulation of this gene, expressed protein and/or use of antibodies or small molecule drugs targeting the gene or gene product are useful in the treatment of inflammatory lung and skin diseases such as asthma, bronchitis, emphysema and psoriasis.

This gene was also expressed at moderate levels in neutrophils stimulated with TNF-a and LPS. The expression of this transcript in neutrophils demonstrates an important role of this receptor in innate immunity and in inflammation associated with neutrophil accummulation such as observed in inflammatory bowel diseases, psoriasis, asthma, chronic bronchitis and rheumatoid arthritis. Therapeutic modulation of this gene, expressed protein and/or use of antibodies or small molecule drugs targeting the gene or gene product are useful in the treatment of inflammatory disorders including inflammatory bowel diseases, psoriasis, asthma, chronic bronchitis and rheumatoid arthritis.

D. CG50389-06: Interleukin-1 Receptor Related Protein

Expression of full-length physical clone CG50389-06 was assessed using the primer-probe sets Ag4944 and Ag4945, described in Tables DA and DB. Results of the RTQ-PCR runs are shown in Table DC.

TABLE DA
Probe Name Ag4944

| | | | | |
|---|---|---|---|---|
| | | | Start | SEQ ID |
| Primers | Sequences | Length | Position | No |

| | | | | |
|---|---|---|---|---|
| Forward | 5′-cagggaagcagtacgaggttt-3′ | 21 | 1161 | 1225 |
| Probe | TET-5′-tggaggaagtgtccctaaaatcatttatcc- | 30 | 1091 | 1226 |
| | 3′-TAMRA | | | |
| Reverse | 5′-tgactctccagcatcgtaga-3′ | 20 | 990 | 1227 |

TABLE DB

Probe Name Ag4945

| | | | | |
|---|---|---|---|---|
| | | | Start | SEQ ID |
| Primers | Sequences | Length | Positon | No |

| | | | | |
|---|---|---|---|---|
| Forward | 5′-cagtgagataccacatgccg-3′ | 20 | 175 | 1228 |
| Probe | TET-5′-agctgccgcagcacacaccttgctac- | 26 | 118 | 1229 |
| | 3′-TAMRA | | | |
| Reverse | 5′-gtcatcttccagccataggtgt-3′ | 22 | 22 | 1230 |

TABLE DC
Panel 4.1D

| Tissue Name | A | B |
|---|---|---|
| Secondary Th1 act | 0.0 | 52.1 |
| Secondary Th2 act | 0.0 | 0.0 |
| Secondary Tr1 act | 0.0 | 0.0 |
| Secondary Th1 rest | 0.0 | 0.0 |
| Secondary Th2 rest | 0.0 | 32.3 |
| Secondary Tr1 rest | 0.0 | 64.2 |
| Primary Th1 act | 0.0 | 12.9 |
| Primary Th2 act | 0.0 | 18.3 |
| Primary Tr1 act | 0.0 | 0.0 |
| Primary Th1 rest | 0.0 | 0.0 |
| Primary Th2 rest | 0.0 | 0.0 |
| Primary Tr1 rest | 0.0 | 17.1 |
| CD45RA CD4 lymphocyte act | 0.0 | 34.2 |
| CD45RO CD4 lymphocyte act | 0.0 | 0.0 |
| CD8 lymphocyte act | 0.0 | 50.7 |
| Secondary CD8 lymphocyte rest | 0.0 | 20.2 |
| Secondary CD8 lymphocyte act | 0.0 | 0.0 |
| CD4 lymphocyte none | 0.0 | 0.0 |
| 2ry Th1/Th2/Tr1_anti-CD95 CH11 | 0.0 | 12.9 |
| LAK cells rest | 0.0 | 20.0 |
| LAK cells IL-2 | 0.0 | 0.0 |
| LAK cells IL-2 + IL-12 | 0.0 | 43.5 |
| LAK cells IL-2 + IFN gamma | 0.0 | 21.6 |
| LAK cells IL-2 + IL-18 | 0.0 | 49.0 |
| LAK cells PMA/ionomycin | 7.7 | 40.3 |
| NK Cells IL-2 rest | 0.0 | 61.1 |
| Two Way MLR 3 day | 0.0 | 12.4 |
| Two Way MLR 5 day | 0.0 | 11.8 |
| Two Way MLR 7 day | 0.0 | 0.0 |
| PBMC rest | 0.0 | 21.3 |
| PBMC PWM | 0.0 | 48.0 |
| PBMC PHA-L | 0.0 | 25.3 |
| Ramos (B cell) none | 0.0 | 0.0 |
| Ramos (B cell) ionomycin | 0.0 | 16.6 |
| B lymphocytes PWM | 10.0 | 35.1 |
| B lymphocytes CD40L and IL-4 | 0.0 | 0.0 |
| EOL-1 dbcAMP | 0.0 | 0.0 |
| EOL-1 dbcAMP PMA/ionomycin | 0.0 | 30.4 |
| Dendritic cells none | 6.9 | 0.0 |
| Dendritic cells LPS | 0.0 | 40.3 |
| Dendritic cells anti-CD40 | 6.2 | 0.0 |
| Monocytes rest | 0.0 | 47.6 |
| Monocytes LPS | 0.0 | 0.0 |
| Macrophages rest | 0.0 | 27.7 |
| Macrophages LPS | 0.0 | 8.9 |
| HUVEC none | 0.0 | 27.9 |
| HUVEC starved | 0.0 | 17.3 |
| HUVEC IL-1beta | 0.0 | 14.7 |
| HUVEC IFN gamma | 0.0 | 17.2 |
| HUVEC TNF alpha + IFN gamma | 0.0 | 0.0 |
| HUVEC TNF alpha + IL4 | 0.0 | 58.6 |
| HUVEC IL-11 | 0.0 | 35.1 |
| Lung Microvascular EC none | 0.0 | 0.0 |
| Lung Microvascular EC TNFalpha + IL-1beta | 0.0 | 37.9 |
| Microvascular Dermal EC none | 0.0 | 19.9 |
| Microsvasular Dermal EC TNFalpha + IL-1beta | 0.0 | 0.0 |
| Bronchial epithelium TNFalpha + IL1beta | 10.7 | 10.1 |
| Small airway epithelium none | 2.7 | 44.8 |
| Small airway epithelium TNFalpha + IL-1beta | 8.1 | 33.4 |
| Coronery artery SMC rest | 0.0 | 18.7 |
| Coronery artery SMC TNFalpha + IL-1beta | 0.0 | 34.9 |
| Astrocytes rest | 0.0 | 18.6 |
| Astrocytes TNFalpha + IL-1beta | 0.0 | 12.4 |
| KU-812 (Basophil) rest | 0.0 | 18.9 |
| KU-812 (Basophil) PMA/ionomycin | 0.0 | 0.0 |
| CCD1106 (Keratinocytes) none | 99.3 | 68.8 |
| CCD1106 (Keratinocytes) TNFalpha + IL-1beta | 100.0 | 21.6 |
| Liver cirrhosis | 0.0 | 0.0 |
| NCI-H292 none | 5.7 | 48.3 |
| NCI-H292 IL-4 | 0.0 | 19.9 |
| NCI-H292 IL-9 | 3.1 | 20.4 |
| NCI-H292 IL-13 | 8.0 | 0.0 |
| NCI-H292 IFN gamma | 3.2 | 0.0 |
| HPAEC none | 0.0 | 55.9 |
| HPAEC TNF alpha + IL-1 beta | 0.0 | 19.1 |
| Lung fibroblast none | 3.9 | 74.7 |
| Lung fibroblast TNF alpha + IL-1 beta | 10.2 | 48.3 |
| Lung fibroblast IL-4 | 19.5 | 44.8 |
| Lung fibroblast IL-9 | 31.9 | 56.6 |
| Lung fibroblast IL-13 | 23.8 | 0.0 |
| Lung fibroblast IFN gamma | 33.2 | 37.9 |
| Dermal fibroblast CCD1070 rest | 0.0 | 100.0 |
| Dermal fibroblast CCD1070 TNF alpha | 4.5 | 24.8 |
| Dermal fibroblast CCD1070 IL-1 beta | 0.0 | 23.2 |
| Dermal fibroblast IFN gamma | 0.0 | 11.8 |
| Dermal fibroblast IL-4 | 12.2 | 46.0 |
| Dermal Fibroblasts rest | 3.8 | 0.0 |
| Neutrophils TNFa + LPS | 23.5 | 51.8 |
| Neutrophils rest | 2.5 | 0.0 |
| Colon | 0.0 | 0.0 |
| Lung | 0.0 | 29.1 |
| Thymus | 0.0 | 8.9 |
| Kidney | 15.3 | 39.2 |

Column A - Rel. Exp. (%) Ag4944, Run 223598842
Column B - Rel. Exp. (%) Ag4945, Run 223625870

Panel 4.1D Summary: 4845/4944 Highest expression of the CG50389-06 transcript was seen in keratinocytes, regardless of their treatment with TNF-a plus IL-1b.

It was also found to a lesser extent in neutrophils and lung fibroblasts. This transcript encodes for a novel IL-1 receptor related protein that may have the potential to trigger novel members of the IL-1 family members as described by Debets and Parnet. (J Immunol Aug. 1, 2001; 167(3):1440-6; J Biol Chem. Feb. 23, 1996; 271(8):3967-70 ). Novel IL1 receptor like molecules have been cloned and reported to lead to activation of NF-kB and IL18 production, a potent inflammatory cytokine associated with lung inflammation, IBD and psoriasis. IL1 R family members have also been shown to mediate inflammatory signals through NF-kB activation, among other signalling pathways. Modulation of the function of this receptor by the use of protein therapeutics or antibodies prevents or reduces the severity of inflammatory processes observed in inflammatory lung and skin diseases such as asthma, bronchitis, emphysema and psoriasis.

E. CG50391-08 and CG50391-09 and CG50391-01:

Expression of gene CG50391-08 was assessed using the primer-probe sets Ag2654, Ag2726, and Ag2826, described in Tables EA, EB, and EC. Results of the RTQ-PCR runs are shown in Tables ED, EE, EF and EG.

TABLE EA
Probe Name Ag2654

| | | | | |
|---|---|---|---|---|
| Primer | Sequences | Length | Start Position | SEQ ID No |

| | | | | |
|---|---|---|---|---|
| Forward | 5′-tagaggagctgctcagcaaata-3′ | 22 | 297 | 1231 |
| Probe | TET-5′-cacaacgagtctcactcccgggt-3′-TAMRA | 23 | 323 | 1232 |
| Reverse | 5′-catgaggatctcctccttgtc-3′ | 21 | 368 | 1233 |

TABLE EB
Probe Name Ag2726

| | | | | |
|---|---|---|---|---|
| Primers | Sequences | Length | Start Position | SEQ ID No |

| | | | | |
|---|---|---|---|---|
| Forward | 5′-tagaggagctgctcagcaaata-3′ | 22 | 297 | 1234 |
| Probe | TET-5′-cacaacgagtctcactcccgggt-3′-TAMRA | 23 | 323 | 1235 |
| Reverse | 5′-catgaggatctcctccttgtc-3′ | 21 | 368 | 1236 |

TABLE EC
Probe Name Ag2826

| | | | | |
|---|---|---|---|---|
| Primers | Sequences | Length | Start Position | SEQ ID No |

| | | | | |
|---|---|---|---|---|
| Forward | 5′-ccctcttactctagcccatttg-3′ | 22 | 2108 | 1237 |
| Probe | TET-5′-tgtctgaccagcaaacattaaaacca-3′-TAMRA | 26 | 2080 | 1238 |
| Reverse | 5′-acagtccgtccctaccaataga-3′ | 22 | 2052 | 1239 |

TABLE ED
Panel 1.3D

| Tissue Name | A | B | C |
|---|---|---|---|
| Liver adenocarcinoma | 5.9 | 3.9 | 4.5 |
| Pancreas | 1.7 | 1.5 | 1.1 |
| Pancreatic ca. CAPAN 2 | 0.1 | 0.2 | 0.2 |
| Adrenal gland | 10.4 | 10.7 | 10.5 |
| Thyroid | 6.3 | 6.9 | 5.6 |
| Salivary gland | 1.0 | 2.5 | 2.6 |
| Pituitary gland | 1.8 | 1.8 | 1.4 |
| Brain (fetal) | 0.2 | 0.4 | 0.3 |
| Brain (whole) | 1.6 | 1.4 | 0.9 |
| Brain (amygdala) | 1.6 | 1.8 | 1.7 |
| Brain (cerebellum) | 0.5 | 0.8 | 0.5 |
| Brain (hippocampus) | 6.2 | 6.3 | 5.0 |
| Brain (substantia nigra) | 1.5 | 1.9 | 0.5 |
| Brain (thalamus) | 1.4 | 1.4 | 2.0 |
| Cerebral Cortex | 0.9 | 1.4 | 1.6 |
| Spinal cord | 8.3 | 6.0 | 6.0 |
| glio/astro U87-MG | 0.0 | 0.1 | 0.0 |
| glio/astro U-118-MG | 4.0 | 3.1 | 2.5 |
| astrocytoma SW1783 | 13.0 | 12.6 | 9.7 |
| neuro*; met SK-N-AS | 1.3 | 1.3 | 0.5 |
| astrocytoma SF-539 | 0.4 | 0.1 | 0.7 |
| astrocytoma SNB-75 | 5.7 | 5.8 | 4.2 |
| glioma SNB-19 | 0.2 | 0.0 | 0.0 |
| glioma U251 | 0.0 | 0.1 | 0.1 |
| glioma SF-295 | 2.4 | 2.4 | 0.8 |
| Heart (fetal) | 17.4 | 19.1 | 9.7 |
| Heart | 5.7 | 6.0 | 4.9 |
| Skeletal muscle (fetal) | 100.0 | 100.0 | 100.0 |
| Skeletal muscle | 0.6 | 0.4 | 0.6 |
| Bone marrow | 5.0 | 6.3 | 5.1 |
| Thymus | 3.6 | 2.8 | 2.4 |
| Spleen | 8.8 | 9.1 | 11.3 |
| Lymph node | 4.7 | 5.6 | 3.8 |
| Colorectal | 27.9 | 29.7 | 44.4 |
| Stomach | 8.7 | 12.9 | 12.2 |
| Small intestine | 16.6 | 8.7 | 23.3 |
| Colon ca. SW480 | 2.0 | 2.5 | 1.4 |
| Colon ca.* SW620(SW480 met) | 0.0 | 0.1 | 0.1 |
| Colon ca. HT29 | 0.3 | 0.0 | 0.2 |
| Colon ca. HCT-116 | 0.4 | 0.7 | 0.6 |
| Colon ca. CaCo-2 | 0.2 | 0.7 | 0.4 |
| Colon ca. tissue(ODO3866) | 20.0 | 24.7 | 17.2 |
| Colon ca. HCC-2998 | 0.3 | 0.6 | 0.7 |
| Gastric ca.* (liver met) NCI-N87 | 0.3 | 0.6 | 1.4 |
| Bladder | 14.2 | 17.8 | 7.9 |
| Trachea | 13.6 | 11.9 | 7.6 |
| Kidney | 1.3 | 1.6 | 1.1 |
| Kidney (fetal) | 8.1 | 9.0 | 4.7 |
| Renal ca. 786-0 | 2.4 | 4.4 | 2.4 |
| Renal ca. A498 | 4.0 | 7.2 | 4.7 |
| Renal ca. RXF 393 | 4.2 | 6.3 | 5.7 |
| Renal ca. ACHN | 79.0 | 79.0 | 38.7 |
| Renal ca. UO-31 | 15.4 | 17.0 | 6.9 |
| Renal ca. TK-10 | 0.3 | 0.3 | 0.1 |
| Liver | 1.1 | 1.0 | 0.7 |
| Liver (fetal) | 2.7 | 5.3 | 4.5 |
| Liver ca. (hepatoblast) HepG2 | 0.0 | 0.0 | 0.0 |
| Lung | 25.0 | 24.5 | 27.4 |
| Lung (fetal) | 29.1 | 26.1 | 22.2 |
| Lung ca. (small cell) LX-1 | 0.8 | 0.0 | 1.1 |
| Lung ca. (small cell) NCI-H69 | 0.4 | 0.0 | 0.8 |
| Lung ca. (s. cell var.) SHP-77 | 2.7 | 4.6 | 1.8 |
| Lung ca. (large cell) NCI-H460 | 0.1 | 0.0 | 0.0 |
| Lung ca. (non-sm. cell) A549 | 0.1 | 0.2 | 0.1 |
| Lung ca. (non-s. cell) NCI-H23 | 0.4 | 0.7 | 0.4 |
| Lung ca. (non-s. cell) HOP-62 | 0.8 | 1.6 | 0.5 |
| Lung ca. (non-s. cl) NCI-H522 | 0.1 | 0.2 | 0.0 |
| Lung ca. (squam.) SW 900 | 0.8 | 1.3 | 2.4 |
| Lung ca. (squam.) NCI-H596 | 0.1 | 0.0 | 0.5 |
| Mammary gland | 16.7 | 11.7 | 10.8 |
| Breast ca.* (pl. ef) MCF-7 | 0.9 | 0.0 | 0.1 |
| Breast ca.* (pl. ef) MDA-MB-231 | 3.3 | 5.2 | 2.9 |
| Breast ca.* (pl. ef) T47D | 1.4 | 0.5 | 2.8 |
| Breast ca. BT-549 | 2.6 | 2.4 | 2.3 |
| Breast ca. MDA-N | 0.0 | 0.0 | 0.0 |
| Ovary | 28.1 | 28.3 | 47.6 |
| Ovarian ca. OVCAR-3 | 7.0 | 7.6 | 5.5 |
| Ovarian ca. OVCAR-4 | 0.0 | 0.4 | 0.2 |
| Ovarian ca. OVCAR-5 | 2.1 | 1.4 | 1.3 |
| Ovarian ca. OVCAR-8 | 0.3 | 0.4 | 0.0 |
| Ovarian ca. IGROV-1 | 0.1 | 0.1 | 0.0 |
| Ovarian ca.* (ascites) SK-OV-3 | 0.0 | 0.1 | 0.0 |
| Uterus | 26.2 | 23.3 | 22.4 |
| Placenta | 27.4 | 29.1 | 32.5 |
| Prostate | 29.5 | 33.2 | 38.2 |
| Prostate ca.* (bone met)PC-3 | 0.1 | 0.0 | 0.1 |
| Testis | 13.4 | 12.2 | 10.5 |
| Melanoma Hs688(A).T | 47.0 | 42.0 | 20.7 |
| Melanoma* (met) Hs688(B).T | 3.6 | 4.5 | 4.2 |
| Melanoma UACC-62 | 0.0 | 0.1 | 0.1 |
| Melanoma M14 | 0.0 | 0.1 | 0.0 |
| Melanoma LOX IMVI | 2.3 | 0.0 | 0.0 |
| Melanoma* (met) SK-MEL-5 | 0.1 | 0.5 | 0.0 |
| Adipose | 81.8 | 77.9 | 44.8 |

Column A - Rel. Exp. (%) Ag2654, Run 153009856
Column B - Rel. Exp. (%) Ag2726, Run 153622446
Column C - Rel. Exp. (%) Ag2826, Run 153774039

TABLE EE
Panel 2D

| Tissue Name | A | B | C |
|---|---|---|---|
| Normal Colon | 52.1 | 75.3 | 58.2 |
| CC Well to Mod Diff (ODO3866) | 18.7 | 19.5 | 15.2 |
| CC Margin (ODO3866) | 18.4 | 18.4 | 13.2 |
| CC Gr.2 rectosigmoid | 8.2 | 10.4 | 2.8 |
| (ODO3868) | | | |
| CC Margin (ODO3868) | 9.0 | 8.2 | 2.3 |
| CC Mod Diff (ODO3920) | 8.7 | 9.2 | 3.6 |
| CC Margin (ODO3920) | 20.2 | 20.9 | 5.0 |
| CC Gr.2 ascend colon | 16.4 | 17.9 | 15.9 |
| (ODO3921) | | | |
| CC Margin (ODO3921) | 16.4 | 18.2 | 11.0 |
| CC from Partial Hepatectomy | 14.4 | 11.7 | 10.4 |
| (ODO4309) Mets | | | |
| Liver Margin (ODO4309) | 8.9 | 6.0 | 7.9 |
| Colon mets to lung (OD04451-01) | 16.5 | 12.2 | 4.5 |
| Lung Margin (OD04451-02) | 33.9 | 28.3 | 9.9 |
| Normal Prostate 6546-1 | 66.0 | 45.1 | 51.8 |
| Prostate Cancer (OD04410) | 30.4 | 30.1 | 31.9 |
| Prostate Margin (OD04410) | 75.8 | 82.9 | 67.8 |
| Prostate Cancer (OD04720-01) | 58.2 | 47.6 | 31.0 |
| Prostate Margin (OD04720-02) | 51.1 | 46.3 | 31.0 |
| Normal Lung 061010 | 16.4 | 17.9 | 20.4 |
| Lung Met to Muscle (ODO4286) | 4.5 | 2.8 | 2.5 |
| Muscle Margin (ODO4286) | 22.2 | 20.7 | 10.3 |
| Lung Malignant Cancer | 10.4 | 6.4 | 9.4 |
| (OD03126) | | | |
| Lung Margin (OD03126) | 15.8 | 16.0 | 12.7 |
| Lung Cancer (OD04404) | 16.6 | 14.1 | 11.8 |
| Lung Margin (OD04404) | 87.7 | 54.3 | 40.1 |
| Lung Cancer (OD04565) | 15.2 | 14.5 | 7.7 |
| Lung Margin (OD04565) | 17.8 | 13.8 | 3.5 |
| Lung Cancer (OD04237-01) | 5.9 | 7.2 | 11.8 |
| Lung Margin (OD04237-02) | 64.6 | 48.3 | 43.8 |
| Ocular Mel Met to Liver | 0.4 | 0.7 | 0.6 |
| (ODO4310) | | | |
| Liver Margin (ODO4310) | 12.6 | 10.6 | 9.8 |
| Melanoma Mets to Lung | 1.6 | 1.1 | 1.1 |
| (OD04321) | | | |
| Lung Margin (OD04321) | 32.8 | 33.2 | 32.8 |
| Normal Kidney | 21.0 | 17.6 | 25.7 |
| Kidney Ca, Nuclear grade 2 | 33.7 | 31.6 | 22.4 |
| (OD04338) | | | |
| Kidney Margin (OD04338) | 48.0 | 39.5 | 14.4 |
| Kidney Ca Nuclear grade 1/2 | 3.1 | 3.1 | 1.4 |
| (OD04339) | | | |
| Kidney Margin (OD04339) | 9.3 | 7.6 | 4.5 |
| Kidney Ca, Clear cell type | 27.4 | 23.7 | 17.0 |
| (OD04340) | | | |
| Kidney Margin (OD04340) | 34.9 | 28.3 | 19.1 |
| Kidney Ca, Nuclear grade 3 | 9.3 | 8.0 | 5.3 |
| (OD04348) | | | |
| Kidney Margin (OD04348) | 27.0 | 23.2 | 10.9 |
| Kidney Cancer (OD04622-01) | 12.7 | 11.6 | 6.9 |
| Kidney Margin (OD04622-03) | 8.4 | 7.4 | 4.5 |
| Kidney Cancer (OD04450-01) | 10.6 | 6.4 | 8.5 |
| Kidney Margin (OD04450-03) | 29.7 | 24.8 | 8.2 |
| Kidney Cancer 8120607 | 3.6 | 4.9 | 2.5 |
| Kidney Margin 8120608 | 12.8 | 16.5 | 6.8 |
| Kidney Cancer 8120613 | 0.4 | 0.0 | 0.1 |
| Kidney Margin 8120614 | 9.9 | 9.4 | 5.4 |
| Kidney Cancer 9010320 | 35.4 | 33.4 | 18.7 |
| Kidney Margin 9010321 | 27.5 | 24.8 | 11.3 |
| Normal Uterus | 20.4 | 24.1 | 12.9 |
| Uterus Cancer 064011 | 50.0 | 48.6 | 12.2 |
| Normal Thyroid | 11.4 | 7.2 | 10.4 |
| Thyroid Cancer 064010 | 1.6 | 2.1 | 2.5 |
| Thyroid Cancer A302152 | 13.8 | 12.5 | 3.5 |
| Thyroid Margin A302153 | 9.0 | 7.7 | 9.9 |
| Normal Breast | 33.2 | 32.5 | 14.9 |
| Breast Cancer (OD04566) | 9.9 | 8.5 | 2.1 |
| Breast Cancer (OD04590-01) | 7.5 | 9.9 | 9.6 |
| Breast Cancer Mets | 14.2 | 14.7 | 12.7 |
| (OD04590-03) | | | |
| Breast Cancer Metastasis | 2.5 | 1.8 | 2.0 |
| (OD04655-05) | | | |
| Breast Cancer 064006 | 20.7 | 19.8 | 18.4 |
| Breast Cancer 1024 | 15.4 | 16.2 | 12.9 |
| Breast Cancer 9100266 | 12.0 | 11.7 | 18.2 |
| Breast Margin 9100265 | 15.2 | 15.2 | 21.8 |
| Breast Cancer A209073 | 10.7 | 10.2 | 12.8 |
| Breast Margin A209073 | 7.7 | 11.1 | 8.2 |
| Normal Liver | 2.1 | 2.0 | 1.9 |
| Liver Cancer 064003 | 1.1 | 1.0 | 1.4 |
| Liver Cancer 1025 | 5.2 | 5.3 | 2.6 |
| Liver Cancer 1026 | 7.3 | 7.6 | 7.7 |
| Liver Cancer 6004-T | 9.3 | 11.6 | 3.4 |
| Liver Tissue 6004-N | 3.8 | 3.8 | 2.6 |
| Liver Cancer 6005-T | 10.2 | 10.7 | 8.0 |
| Liver Tissue 6005-N | 4.1 | 3.9 | 2.1 |
| Normal Bladder | 37.1 | 43.2 | 35.1 |
| Bladder Cancer 1023 | 11.3 | 9.2 | 11.2 |
| Bladder Cancer A302173 | 4.4 | 5.6 | 10.2 |
| Bladder Cancer (OD04718-01) | 28.1 | 30.6 | 27.2 |
| Bladder Normal Adjacent | 100.0 | 100.0 | 78.5 |
| (OD04718-03) | | | |
| Normal Ovary | 10.4 | 8.1 | 10.2 |
| Ovarian Cancer 064008 | 92.7 | 95.9 | 100.0 |
| Ovarian Cancer (OD04768-07) | 1.9 | 1.7 | 1.4 |
| Ovary Margin (OD04768-08) | 71.2 | 68.3 | 32.8 |
| Normal Stomach | 70.7 | 69.7 | 43.5 |
| Gastric Cancer 9060358 | 12.8 | 16.5 | 24.3 |
| Stomach Margin 9060359 | 23.7 | 25.2 | 21.2 |
| Gastric Cancer 9060395 | 25.2 | 32.5 | 32.5 |
| Stomach Margin 9060394 | 43.2 | 49.0 | 55.9 |
| Gastric Cancer 9060397 | 28.1 | 26.4 | 29.3 |
| Stomach Margin 9060396 | 8.7 | 10.2 | 9.2 |
| Gastric Cancer 064005 | 18.6 | 26.4 | 33.9 |

Column A - Rel. Exp. (%) Ag2654, Run 153009865
Column B - Rel. Exp. (%) Ag2726, Run 153622484
Column C - Rel. Exp. (%) Ag2826, Run 153774078

TABLE EF
Panel 4D

| Tissue Name | A | B | C |
|---|---|---|---|
| Secondary Th1 act | 0.3 | 0.5 | 0.0 |
| Secondary Th2 act | 0.0 | 0.0 | 0.0 |
| Secondary Tr1 act | 0.1 | 0.0 | 0.0 |
| Secondary Th1 rest | 0.0 | 0.0 | 0.0 |
| Secondary Th2 rest | 0.1 | 0.1 | 0.0 |
| Secondary Tr1 rest | 0.0 | 0.0 | 0.0 |
| Primary Th1 act | 0.1 | 0.0 | 0.0 |
| Primary Th2 act | 0.1 | 0.1 | 0.0 |
| Primary Tr1 act | 0.0 | 0.0 | 0.0 |
| Primary Th1 rest | 0.1 | 0.2 | 0.2 |
| Primary Th2 rest | 0.0 | 0.1 | 0.1 |
| Primary Tr1 rest | 0.0 | 0.0 | 0.0 |
| CD45RA CD4 lymphocyte act | 24.7 | 20.9 | 15.3 |
| CD45RO CD4 lymphocyte act | 0.2 | 0.1 | 0.1 |
| CD8 lymphocyte act | 0.0 | 0.1 | 0.0 |
| Secondary CD8 lymphocyte rest | 0.0 | 0.0 | 0.0 |
| Secondary CD8 lymphocyte act | 0.3 | 0.3 | 0.0 |
| CD4 lymphocyte none | 0.4 | 0.1 | 0.2 |
| 2ry Th1/Th2/Tr1 anti-CD95 CH11 | 0.0 | 0.2 | 0.0 |
| LAK cells rest | 1.4 | 2.7 | 2.0 |
| LAK cells IL-2 | 0.2 | 0.2 | 0.0 |
| LAK cells IL-2 + IL-12 | 0.1 | 0.3 | 0.1 |
| LAK cells IL-2 + IFN gamma | 0.4 | 0.3 | 0.2 |
| LAK cells IL-2 + IL-18 | 0.4 | 0.0 | 0.2 |
| LAK cells PMA/ionomycin | 2.2 | 1.0 | 1.0 |
| NK Cells IL-2 rest | 0.0 | 0.4 | 0.0 |
| Two Way MLR 3 day | 2.2 | 3.6 | 1.9 |
| Two Way MLR 5 day | 0.8 | 1.7 | 0.8 |
| Two Way MLR 7 day | 0.1 | 0.4 | 0.1 |
| PBMC rest | 3.0 | 3.6 | 3.9 |
| PBMC PWM | 3.8 | 3.1 | 4.1 |
| PBMC PHA-L | 4.3 | 3.6 | 5.9 |
| Ramos (B cell) none | 0.9 | 1.2 | 2.5 |
| Ramos (B cell) ionomycin | 1.0 | 1.1 | 3.5 |
| B lymphocytes PWM | 0.3 | 0.5 | 0.0 |
| B lymphocytes CD40L and IL-4 | 0.6 | 0.4 | 0.1 |
| EOL-1 dbcAMP | 0.2 | 0.1 | 0.1 |
| EOL-1 dbcAMP PMA/ionomycin | 0.0 | 0.0 | 0.1 |
| Dendritic cells none | 0.2 | 0.2 | 0.2 |
| Dendritic cells LPS | 0.4 | 0.5 | 0.3 |
| Dendritic cells anti-CD40 | 0.0 | 0.1 | 0.0 |
| Monocytes rest | 31.0 | 31.9 | 33.4 |
| Monocytes LPS | 1.4 | 2.2 | 1.3 |
| Macrophages rest | 0.1 | 0.7 | 0.6 |
| Macrophages LPS | 4.8 | 6.3 | 5.3 |
| HUVEC none | 0.0 | 0.0 | 0.0 |
| HUVEC starved | 0.3 | 0.0 | 0.0 |
| HUVEC IL-1beta | 0.0 | 0.0 | 0.0 |
| HUVEC IFN gamma | 0.1 | 0.6 | 0.1 |
| HUVEC TNF alpha + IFN gamma | 0.0 | 0.0 | 0.0 |
| HUVEC TNF alpha + IL4 | 0.0 | 0.0 | 0.0 |
| HUVEC IL-11 | 0.2 | 0.0 | 0.0 |
| Lung Microvascular EC none | 0.0 | 0.0 | 0.0 |
| Lung Microvascular EC | 0.0 | 0.1 | 0.0 |
| TNFalpha + IL-1beta | | | |
| Microvascular Dermal EC none | 1.4 | 1.6 | 1.1 |
| Microsvasular Dermal EC | 0.1 | 0.0 | 0.1 |
| TNFalpha + IL-1 beta | | | |
| Bronchial epithelium TNFalpha + | 0.0 | 0.0 | 0.1 |
| IL1beta | | | |
| Small airway epithelium none | 1.6 | 1.0 | 1.6 |
| Small airway epithelium | 2.2 | 3.5 | 6.4 |
| TNFalpha + IL-1beta | | | |
| Coronery artery SMC rest | 0.3 | 0.3 | 0.5 |
| Coronery artery SMC TNFalpha + | 0.1 | 0.4 | 0.0 |
| IL-1beta | | | |
| Astrocytes rest | 18.9 | 20.6 | 21.2 |
| Astrocytes TNFalpha + IL-1beta | 9.0 | 8.3 | 11.6 |
| KU-812 (Basophil) rest | 0.0 | 0.1 | 0.1 |
| KU-812 (Basophil) PMA/ionomycin | 0.6 | 0.0 | 0.1 |
| CCD1106 (Keratinocytes) none | 0.3 | 0.4 | 0.5 |
| CCD1106 (Keratinocytes) | 0.0 | 0.0 | 0.1 |
| TNFalpha + IL-1 beta | | | |
| Liver cirrhosis | 18.3 | 22.4 | 12.5 |
| Lupus kidney | 10.3 | 9.6 | 9.9 |
| NCI-H292 none | 0.5 | 0.3 | 1.3 |
| NCI-H292 IL-4 | 0.7 | 0.3 | 1.4 |
| NCI-H292 IL-9 | 0.5 | 0.7 | 1.9 |
| NCI-H292 IL-13 | 0.6 | 0.1 | 0.7 |
| NCI-H292 IFN gamma | 0.5 | 0.4 | 0.4 |
| HPAEC none | 0.0 | 0.0 | 0.0 |
| HPAEC TNF alpha + IL-1 beta | 0.0 | 0.4 | 0.0 |
| Lung fibroblast none | 12.0 | 12.2 | 10.4 |
| Lung fibroblast TNF alpha + IL-1 | 8.3 | 9.7 | 11.5 |
| beta | | | |
| Lung fibroblast IL-4 | 17.8 | 16.4 | 25.0 |
| Lung fibroblast IL-9 | 16.3 | 16.2 | 16.8 |
| Lung fibroblast IL-13 | 14.2 | 13.9 | 13.9 |
| Lung fibroblast IFN gamma | 29.9 | 33.0 | 40.9 |
| Dermal fibroblast CCD1070 rest | 89.5 | 100.0 | 100.0 |
| Dermal fibroblast CCD1070 TNF | 63.3 | 51.4 | 76.8 |
| alpha | | | |
| Dermal fibroblast CCD1070 IL-1 | 100.0 | 94.0 | 70.7 |
| beta | | | |
| Dermal fibroblast IFN gamma | 9.7 | 9.8 | 7.9 |
| Dermal fibroblast IL-4 | 2.8 | 1.6 | 1.8 |
| IBD Colitis 2 | 22.8 | 30.4 | 22.4 |
| IBD Crohn's | 11.2 | 14.7 | 10.4 |
| Colon | 18.2 | 22.1 | 16.6 |
| Lung | 51.8 | 46.3 | 40.6 |
| Thymus | 11.2 | 10.3 | 11.9 |
| Kidney | 10.6 | 8.7 | 8.0 |

Column A - Rel. Exp. (%) Ag2654, Run 153594146
Column B - Rel. Exp. (%) Ag2726, Run 153622627
Column C - Rel. Exp. (%) Ag2826, Run 153774117

TABLE EG
Panel 5D

| Tissue Name | A | B |
|---|---|---|
| 97457_Patient-02go_adipose | 100.0 | 74.7 |
| 97476_Patient-07sk_skeletal muscle | 37.4 | 31.9 |
| 97477_Patient-07ut_uterus | 83.5 | 95.3 |
| 97478_Patient-07pl_placenta | 22.4 | 22.5 |
| 97481_Patient-08sk_skeletal muscle | 12.2 | 8.2 |
| 97482_Patient-08ut_uterus | 46.3 | 48.6 |
| 97483_Patient-08pl_placenta | 19.5 | 22.5 |
| 97486_Patient-09sk_skeletal muscle | 5.8 | 7.5 |
| 97487_Patient-09ut_uterus | 19.9 | 18.7 |
| 97488_Patient-09pl_placenta | 13.2 | 24.8 |
| 97492_Patient-10ut_uterus | 29.7 | 19.9 |
| 97493_Patient-10pl_placenta | 31.0 | 30.4 |
| 97495_Patient-11go_adipose | 45.1 | 31.4 |
| 97496_Patient-11sk_skeletal muscle | 11.0 | 5.0 |
| 97497_Patient-11ut_uterus | 66.9 | 57.0 |
| 97498_Patient-11pl_placenta | 11.5 | 21.3 |
| 97500_Patient-12go_adipose | 62.0 | 54.7 |
| 97501_Patient-12sk_skeletal muscle | 15.5 | 25.9 |
| 97502_Patient-12ut_uterus | 90.1 | 62.0 |
| 97503_Patient-12pl_placenta | 11.6 | 11.6 |
| 94721_Donor 2 U- | 31.9 | 10.9 |
| A_Mesenchymal Stem Cells | | |
| 94722_Donor 2 U - | 15.6 | 16.6 |
| B_Mesenchymal Stem Cells | | |
| 94723_Donor 2 U - | 18.6 | 25.3 |
| C_Mesenchymal Stem Cells | | |
| 94709_Donor 2 AM - A_adipose | 81.8 | 100.0 |
| 94710_Donor 2 AM - B_adipose | 77.4 | 84.1 |
| 94711_Donor 2 AM - C_adipose | 66.4 | 33.9 |
| 94712_Donor 2 AD - A_adipose | 15.5 | 14.8 |
| 94713_Donor 2 AD - B_adipose | 16.6 | 18.8 |
| 94714_Donor 2 AD - C_adipose | 22.5 | 22.5 |
| 94742_Donor 3 U - A_Mesenchymal | 33.9 | 21.6 |
| Stem Cells | | |
| 94743_Donor 3 U - B_Mesenchymal | 27.0 | 25.2 |
| Stem Cells | | |
| 94730_Donor 3 AM - A_adipose | 75.3 | 65.1 |
| 94731_Donor 3 AM - B_adipose | 52.1 | 80.1 |
| 94732_Donor 3 AM - C_adipose | 42.0 | 35.6 |
| 94733_Donor 3 AD - A_adipose | 12.1 | 9.9 |
| 94734_Donor 3 AD - B_adipose | 0.2 | 14.5 |
| 94735_Donor 3 AD - C_adipose | 10.7 | 13.0 |
| 77138_Liver_HepG2untreated | 1.3 | 2.6 |
| 73556_Heart_Cardiac stromal cells | 0.2 | 0.0 |
| (primary) | | |
| 81735_Small Intestine | 21.0 | 27.2 |
| 72409_Kidney_Proximal Convoluted | 2.7 | 2.8 |
| Tubule | | |
| 82685_Small intestine_Duodenum | 10.2 | 13.1 |
| 90650_Adrenal_Adrenocortical | 11.0 | 15.8 |
| adenoma | | |
| 72410_Kidney_HRCE | 25.2 | 56.3 |
| 72411_Kidney_HRE | 7.6 | 7.0 |
| 73139_Uterus_Uterine smooth muscle | 1.3 | 3.1 |
| cells | | |

Column A - Rel. Exp. (%) Ag2654, Run 169270118
Column B - Rel. Exp. (%) Ag2726, Run 169315037

Panel 1.3D Summary: Ag2654/Ag2726/Ag2826 Highest expression of this gene was detected in fetal skeletal muscle (CTs=27-28.2). This gene was expressed at much higher levels in fetal when compared to adult skeletal muscle (CT=35). The relative overexpression of this gene in fetal skeletal muscle demonstrates that the gene or protein product enhances muscular growth or development in the fetus and also acts in a regenerative capacity in the adult.

Moderate to low levels of expression of this gene were also seen in tissues with - metabolic/endocrine-functions-including pancreas,- adipose, adrenal gland, thyroid, pituitary gland, skeletal muscle, heart, liver and the gastrointestinal tract. -Therapeutic modulation of the activity of this gene or gene product is useful in the treatment of endocrine/metabolically related diseases, such as obesity and diabetes.

Moderate expression of this gene was also seen in number of cell lines derived from melanoma, liver, renal, breast, lung, ovarian, and brain cancers.

Panel 2D Summary: Ag2654/Ag2726/Ag2826 Highest expression of this gene was detected in a normal bladder sample (CTs=26-27.5). Significant expression of this gene was seen in both normal and cancer samples used in this panel. Expression of this gene was higher in normal lung, bladder and breast samples as compared to their corresponding cancer samples.

Panel 4D Summary: Ag2654/Ag2726/Ag2826 Highest expression of this gene was detected in dermal fibroblasts (CTs=26.4-28). High to moderate expression of this gene was detected in dermal and lung fibroblasts, activated CD45RA CD4 lymphocyte cells, two way MLR, PBMC cells, resting monocytes, LPS treated macrophage, small airway epithelium, liver cirrhosis, lupus kidney, IBD colon, and normal tissues represented by colon, lung, thymus and kidney.

Panel 5D Summary: Ag2654/Ag2726 Highest expression of this gene was seen in adipose (CTs=30). This gene showed ubiquitous expression in this panel, with moderate expression in adipose, skeletal muscle, small intesting, uterus, placenta, kidney, and mesenchmyal stem cells.

F. CG50426-15 and CG50426-17 and CG50426-21: NEURESTIN

Expression of full-length physical clone CG50426-15 and variants CG50426-17 and CG50426-21 was assessed using the primer-probe sets Ag2679, Ag2728, Ag2975, Ag332, Ag047, and Ag47b, described in Tables FA, FB, FC, FD, FE, and FF. Results of the RTQ-PCR runs are shown in Tables FG, FH, Fl and FJ. The CG50426-15 variant only corresponds to Ag332 and Ag47b.

TABLE FA
Probe Name Ag2679

| | | | | |
|---|---|---|---|---|
| Primers | Sequences | Length | Start Position | SEQ ID No |

| | | | | |
|---|---|---|---|---|
| Forward | 5′-ttgacctcaggaacggtttac-3′ | 21 | 1136 | 1240 |
| Probe | TET-5′-ctgctgcccaggaatactttctccag-3′-TAMRA | 26 | 1172 | 1241 |
| Reverse | 5′-agtatttggagggcttcttcag-3′ | 22 | 1211 | 1242 |

TABLE FB
Probe Name Ag2728

| | | | | |
|---|---|---|---|---|
| Primers | Sequences | Length | Start Position | SEQ ID No |

| | | | | |
|---|---|---|---|---|
| Forward | 5′-ggagcttggtctcatgaccta-3′ | 21 | 4810 | 1243 |
| Probe | TET-5′-actgggctcctggccaccaagag-3′-TAMRA | 23 | 4841 | 1244 |
| Reverse | 5′-agtcgtccatcctgtttcatc-3′ | 21 | 4865 | 1245 |

TABLE FC
Probe Name Ag2975

| | | | | |
|---|---|---|---|---|
| Primers | Sequences | Length | Start Position | SEQ ID No |

| | | | | |
|---|---|---|---|---|
| Forward | 5′-ggagcttggtctcatgaccta-3′ | 21 | 4810 | 1246 |
| Probe | TET-5′-actgggctcctggccaccaagag-3′-TAMRA | 23 | 4841 | 1247 |
| Reverse | 5′-agtcgtccatcctgtttcatc-3′ | 21 | 4865 | 1248 |

TABLE FD
Probe Name Ag332

| | | | | |
|---|---|---|---|---|
| Primers | Sequences | Length | Start Position | SEQ ID No |

| | | | | |
|---|---|---|---|---|
| Forward | 5′-gctgccctgacttgtgcaa-3′ | 19 | 2211 | 1249 |
| Probe | TET-5′-tctgacccagtgtgcatctcccgtt-3′-TAMRA | 25 | 2234 | 1250 |
| Reverse | 5′-ccggtctggcagacacact-3′ | 19 | 2268 | 1251 |

TABLE FE
Probe Name Ag047

| | | | | |
|---|---|---|---|---|
| Primers | Sequences | Length | Start Position | SEQ ID No |

| | | | | |
|---|---|---|---|---|
| Forward | 5′-ccaatgacctggccacca-3′ | 18 | 987 | 1252 |
| Probe | TET-5′-ccagagtccgttcagcttcaggacagc-3′-TAMRA | 27 | 1007 | 1253 |
| Reverse | 5′-gtggcacgttgctgtttagc-3′ | 20 | 1039 | 1254 |

TABLE FF
Probe Name Ag47b

| | | | | |
|---|---|---|---|---|
| Primers | Sequences | Length | Start Position | SEQ ID No |

| | | | | |
|---|---|---|---|---|
| Forward | 5′-gaacgccggagcatacaga-3′ | 19 | 1574 | 1255 |
| Probe | TET-5′-ccaggtactgcacaaacacggcttcat-3′-TAMRA | 27 | 1605 | 1256 |
| Reverse | 5′-gatgccacaggcccaca-3′ | 17 | 1633 | 1257 |

TABLE FG
Panel 1

| Tissue Name | A | B | C | D | E |
|---|---|---|---|---|---|
| Endothelial cells | 0.0 | 0.0 | 0.0 | 0.0 | 0.0 |
| Endothelial cells (treated) | 0.0 | 0.0 | 0.0 | 0.0 | 0.0 |
| Pancreas | 0.0 | 0.3 | 0.0 | 0.0 | 0.0 |
| Pancreatic ca. CAPAN 2 | 0.0 | 0.0 | 0.0 | 0.0 | 0.0 |
| Adrenal gland | 0.0 | 1.3 | 0.2 | 0.0 | 0.0 |
| Thyroid | 0.0 | 0.4 | 100.0 | 0.1 | 0.0 |
| Salivary gland | 0.0 | 0.2 | 0.0 | 0.0 | 0.0 |
| Pituitary gland | 0.0 | 0.1 | 2.1 | 0.0 | 0.0 |
| Brain (fetal) | 0.0 | 15.0 | 3.4 | 11.0 | 21.8 |
| Brain (whole) | 95.9 | 67.8 | 3.2 | 4.5 | 32.5 |
| Brain (amygdala) | 0.0 | 8.8 | 3.8 | 8.7 | 12.2 |
| Brain (cerebellum) | 0.0 | 22.2 | 1.4 | 0.1 | 14.3 |
| Brain (hippocampus) | 0.0 | 24.7 | 3.4 | 8.7 | 15.6 |
| Brain (substantia nigra) | 3.4 | 3.6 | 1.3 | 2.3 | 3.8 |
| Brain (thalamus) | 3.5 | 4.7 | 3.3 | 5.0 | 9.1 |
| Brain (hypothalamus) | 0.0 | 0.7 | 0.2 | 0.0 | 0.0 |
| Spinal cord | 0.7 | 1.5 | 0.9 | 1.4 | 1.1 |
| glio/astro U87-MG | 0.6 | 2.6 | 1.6 | 1.7 | 3.0 |
| glio/astro U-118-MG | 0.0 | 0.6 | 0.2 | 1.1 | 0.1 |
| astrocytoma SW1783 | 33.7 | 29.5 | 9.0 | 42.9 | 36.1 |
| neuro*; met SK-N-AS | 0.0 | 0.0 | 0.0 | 0.0 | 0.0 |
| astrocytoma SF-539 | 31.0 | 36.9 | 10.3 | 68.3 | 48.3 |
| astrocytoma SNB-75 | 33.7 | 32.8 | 7.1 | 23.0 | 50.3 |
| glioma SNB-19 | 100.0 | 100.0 | 30.1 | 100.0 | 100.0 |
| glioma U251 | 49.0 | 44.1 | 16.5 | 57.8 | 41.5 |
| glioma SF-295 | 6.0 | 8.1 | 2.1 | 19.8 | 8.7 |
| Heart | 61.1 | 26.8 | 36.6 | 70.7 | 39.8 |
| Skeletal muscle | 0.0 | 0.1 | 0.2 | 0.0 | 0.0 |
| Bone marrow | 0.0 | 0.1 | 0.0 | 0.0 | 0.0 |
| Thymus | 18.7 | 18.4 | 1.1 | 2.7 | 17.2 |
| Spleen | 0.0 | 0.0 | 0.0 | 0.0 | 0.0 |
| Lymph node | 0.0 | 0.2 | 0.0 | 0.0 | 0.0 |
| Colon (ascending) | 0.5 | 0.7 | 0.7 | 0.9 | 4.5 |
| Stomach | 0.1 | 1.1 | 0.2 | 0.2 | 0.2 |
| Small intestine | 0.0 | 0.1 | 0.0 | 0.0 | 0.0 |
| Colon ca. SW480 | 0.6 | 1.0 | 1.6 | 7.0 | 1.4 |
| Colon ca.* SW620 (SW480 met) | 0.0 | 0.0 | 0.0 | 0.0 | 0.0 |
| Colon ca. HT29 | 0.0 | 0.0 | 0.0 | 0.0 | 0.0 |
| Colon ca. HCT-116 | 0.0 | 0.0 | 0.0 | 0.0 | 0.0 |
| Colon ca. CaCo-2 | 0.0 | 0.1 | 0.0 | 0.0 | 0.0 |
| Colon ca. HCT-15 | 0.0 | 0.1 | 0.2 | 0.0 | 0.0 |
| Colon ca. HCC-2998 | 0.0 | 0.0 | 0.0 | 0.0 | 0.0 |
| Gastric ca. * (liver met) NCI-N87 | 0.0 | 0.4 | 0.2 | 0.0 | 0.0 |
| Bladder | 0.3 | 1.0 | 0.2 | 0.0 | 0.1 |
| Trachea | 0.0 | 0.4 | 0.2 | 0.0 | 0.0 |
| Kidney | 0.2 | 0.9 | 0.7 | 1.2 | 1.0 |
| Kidney (fetal) | 1.3 | 3.9 | 0.8 | 3.2 | 2.7 |
| Renal ca. 786-0 | 10.6 | 11.7 | 3.4 | 7.9 | 12.2 |
| Renal ca. A498 | 0.0 | 0.1 | 0.0 | 0.0 | 0.0 |
| Renal ca. RXF 393 | 17.9 | 14.0 | 4.1 | 16.6 | 15.2 |
| Renal ca. ACHN | 0.0 | 0.0 | 0.0 | 0.0 | 0.0 |
| Renal ca. UO-31 | 0.0 | 0.2 | 0.1 | 0.0 | 0.2 |
| Renal ca. TK-10 | 0.0 | 0.0 | 0.0 | 0.0 | 0.0 |
| Liver | 0.0 | 3.2 | 0.8 | 0.9 | 1.9 |
| Liver (fetal) | 0.0 | 0.1 | 0.0 | 0.0 | 0.0 |
| Liver ca. (hepatoblast) HepG2 | 0.0 | 0.0 | 0.0 | 0.0 | 0.0 |
| Lung | 0.0 | 0.2 | 0.0 | 0.0 | 0.0 |
| Lung (fetal) | 0.0 | 0.5 | 0.0 | 0.0 | 0.0 |
| Lung ca. (small cell) LX-1 | 0.0 | 0.0 | 0.0 | 0.0 | 0.0 |
| Lung ca. (small cell) NCI-H69 | 2.0 | 3.3 | 1.4 | 5.4 | 2.9 |
| Lung ca. (s. cell var.) SHP-77 | 0.0 | 0.0 | 0.0 | 0.0 | 0.0 |
| Lung ca. (large cell)NCI-H460 | 0.0 | 0.0 | 1.3 | 0.7 | 0.3 |
| Lung ca. (non-sm. cell) A549 | 0.0 | 0.0 | 0.1 | 0.0 | 0.0 |
| Lung ca. (non-s. cell) NCI-H23 | 0.0 | 0.0 | 0.0 | 0.0 | 0.0 |
| Lung ca. (non-s. cell) HOP-62 | 5.2 | 4.7 | 3.9 | 11.3 | 6.7 |
| Lung ca. (non-s. cl) NCI-H522 | 1.7 | 3.1 | 1.5 | 2.8 | 4.8 |
| Lung ca. (squam.) SW 900 | 0.0 | 0.0 | 0.0 | 0.0 | 0.0 |
| Lung ca. (squam.) NCI-H596 | 1.3 | 2.5 | 2.0 | 4.2 | 3.0 |
| Mammary gland | 11.9 | 9.1 | 4.6 | 7.0 | 10.6 |
| Breast ca.* (pl. ef) MCF-7 | 0.0 | 0.0 | 0.0 | 0.0 | 0.0 |
| Breast ca.* (pl. ef) MDA-MB-231 | 0.0 | 0.1 | 0.0 | 0.0 | 0.0 |
| Breast ca.* (pl. ef) T47D | 11.8 | 7.5 | 7.9 | 31.6 | 31.4 |
| Breast ca. BT-549 | 0.0 | 0.0 | 4.5 | 13.8 | 28.3 |
| Breast ca. MDA-N | 0.0 | 0.1 | 0.0 | 0.0 | 0.0 |
| Ovary | 0.1 | 0.6 | 0.4 | 0.1 | 0.2 |
| Ovarian ca. OVCAR-3 | 0.0 | 0.0 | 0.0 | 0.0 | 0.0 |
| Ovarian ca. OVCAR-4 | 0.0 | 0.1 | 1.3 | 4.4 | 0.0 |
| Ovarian ca. OVCAR-5 | 73.2 | 38.2 | 14.4 | 57.0 | 36.1 |
| Ovarian ca. OVCAR-8 | 0.0 | 0.7 | 1.0 | 3.4 | 0.2 |
| Ovarian ca. IGROV-1 | 0.0 | 0.0 | 0.8 | 2.6 | 0.0 |
| Ovarian ca. (ascites) SK-OV-3 | 0.0 | 0.0 | 0.0 | 0.0 | 0.0 |
| Uterus | 0.4 | 1.2 | 0.6 | 1.7 | 2.9 |
| Placenta | 0.0 | 0.1 | 0.0 | 0.0 | 0.0 |
| Prostate | 0.9 | 1.9 | 0.7 | 1.9 | 1.7 |
| Prostate ca.* (bone met) PC-3 | 0.0 | 0.0 | 0.1 | 0.0 | 0.0 |
| Testis | 25.7 | 22.2 | 0.7 | 0.7 | 26.4 |
| Melanoma Hs688(A).T | 23.8 | 21.6 | 5.0 | 20.0 | 28.9 |
| Melanoma* (met) Hs688(B).T | 4.6 | 6.5 | 3.4 | 9.6 | 9.2 |
| Melanoma UACC-62 | 0.0 | 0.0 | 0.0 | 0.0 | 0.0 |
| Melanoma M14 | 0.0 | 0.1 | 0.0 | 0.0 | 0.0 |
| Melanoma LOX IMVI | 3.7 | 3.4 | 0.6 | 0.2 | 2.7 |
| Melanoma* (met) SK-MEL-5 | 0.0 | 0.0 | 0.0 | 0.0 | 0.0 |
| Melanoma SK-MEL-28 | 0.0 | 0.0 | 3.6 | 17.4 | 0.0 |

Column A - Rel. Exp. (%) Ag047, Run 87354354
Column B - Rel. Exp. (%) Ag047, Run 87354779
Column C - Rel. Exp. (%) Ag332, Run 97803603
Column D - Rel. Exp. (%) Ag332, Run 98747043
Column E - Rel. Exp. (%) Ag47b, Run 88164379

TABLE FH
Panel 2D

| Tissue Name | A | B | C | D |
|---|---|---|---|---|
| Normal Colon | 5.5 | 8.9 | 7.4 | 10.4 |
| CC Well to Mod Diff (ODO3866) | 1.4 | 0.2 | 0.2 | 1.3 |
| CC Margin (ODO3866) | 0.4 | 0.5 | 0.4 | 0.1 |
| CC Gr.2 rectosigmoid (ODO3868) | 0.4 | 0.1 | 1.7 | 0.2 |
| CC Margin (ODO3868) | 2.5 | 0.9 | 0.7 | 0.7 |
| CC Mod Diff (ODO3920) | 0.0 | 0.0 | 0.0 | 0.2 |
| CC Margin (ODO3920) | 0.7 | 0.9 | 1.1 | 0.3 |
| CC Gr.2 ascend colon (ODO3921) | 0.0 | 0.7 | 0.6 | 0.2 |
| CC Margin (ODO3921) | 0.2 | 0.2 | 0.6 | 0.8 |
| CC from Partial Hepatectomy | 0.6 | 0.4 | 1.1 | 1.0 |
| (ODO4309) Mets | | | | |
| Liver Margin (ODO4309) | 11.1 | 13.6 | 30.4 | 21.2 |
| Colon mets to lung (OD04451-01) | 0.0 | 0.4 | 0.1 | 0.2 |
| Lung Margin (OD04451-02) | 1.3 | 1.0 | 0.5 | 0.7 |
| Normal Prostate 6546-1 | 19.1 | 0.7 | 1.0 | 2.3 |
| Prostate Cancer (OD04410) | 5.8 | 2.9 | 4.7 | 3.4 |
| Prostate Margin (OD04410) | 4.7 | 5.7 | 7.2 | 4.9 |
| Prostate Cancer (OD04720-01) | 2.9 | 3.3 | 3.4 | 3.3 |
| Prostate Margin (OD04720-02) | 12.5 | 10.7 | 8.3 | 15.2 |
| Normal Lung 061010 | 1.5 | 2.7 | 6.8 | 5.4 |
| Lung Met to Muscle (ODO4286) | 0.3 | 0.1 | 0.0 | 0.5 |
| Muscle Margin (ODO4286) | 0.3 | 0.2 | 0.3 | 0.6 |
| Lung Malignant Cancer (OD03126) | 0.4 | 0.2 | 0.8 | 1.5 |
| Lung Margin (OD03126) | 0.4 | 1.0 | 1.2 | 0.5 |
| Lung Cancer (OD04404) | 86.5 | 100.0 | 100.0 | 100.0 |
| Lung Margin (OD04404) | 18.3 | 3.3 | 2.2 | 3.0 |
| Lung Cancer (OD04565) | 100.0 | 52.1 | 62.0 | 77.9 |
| Lung Margin (OD04565) | 0.2 | 0.1 | 0.0 | 0.6 |
| Lung Cancer (OD04237-01) | 6.3 | 1.5 | 3.2 | 3.3 |
| Lung Margin (OD04237-02) | 1.4 | 0.5 | 0.5 | 0.6 |
| Ocular Mel Met to Liver (ODO4310) | 0.4 | 0.3 | 0.5 | 0.7 |
| Liver Margin (ODO4310) | 2.3 | 1.8 | 3.5 | 2.8 |
| Melanoma Mets to Lung (OD04321) | 0.0 | 0.3 | 0.4 | 1.6 |
| Lung Margin (OD04321) | 2.1 | 2.6 | 2.2 | 3.0 |
| Normal Kidney | 6.7 | 4.9 | 8.4 | 7.0 |
| Kidney Ca, Nuclear grade 2 | 0.0 | 0.0 | 0.3 | 1.4 |
| (OD04338) | | | | |
| Kidney Margin (OD04338) | 3.5 | 1.5 | 4.1 | 3.5 |
| Kidney Ca Nuclear grade 1/2 | 0.0 | 0.1 | 0.3 | 0.7 |
| (OD04339) | | | | |
| Kidney Margin (OD04339) | 18.4 | 10.3 | 8.4 | 15.5 |
| Kidney Ca, Clear cell type | 0.0 | 0.8 | 0.7 | 1.1 |
| (OD04340) | | | | |
| Kidney Margin (OD04340) | 6.5 | 4.4 | 4.5 | 6.5 |
| Kidney Ca, Nuclear grade 3 | 90.8 | 36.1 | 54.3 | 50.3 |
| (OD04348) | | | | |
| Kidney Margin (OD04348) | 4.6 | 3.2 | 3.8 | 4.0 |
| Kidney Cancer (OD04622-01) | 2.7 | 1.8 | 4.1 | 3.5 |
| Kidney Margin (OD04622-03) | 2.0 | 0.2 | 0.3 | 1.1 |
| Kidney Cancer (OD04450-01) | 0.0 | 0.0 | 0.0 | 0.0 |
| Kidney Margin (OD04450-03) | 3.1 | 1.4 | 6.9 | 5.4 |
| Kidney Cancer 8120607 | 1.5 | 0.3 | 0.5 | 1.8 |
| Kidney Margin 8120608 | 3.4 | 0.8 | 1.3 | 2.2 |
| Kidney Cancer 8120613 | 0.4 | 0.8 | 4.3 | 2.0 |
| Kidney Margin 8120614 | 2.8 | 1.2 | 4.9 | 4.0 |
| Kidney Cancer 9010320 | 76.8 | 39.0 | 36.3 | 52.9 |
| Kidney Margin 9010321 | 10.4 | 5.1 | 4.9 | 4.3 |
| Normal Uterus | 0.0 | 0.2 | 0.3 | 0.0 |
| Uterus Cancer 064011 | 0.9 | 0.0 | 0.3 | 1.9 |
| Normal Thyroid | 0.3 | 0.0 | 0.9 | 2.2 |
| Thyroid Cancer 064010 | 0.0 | 0.0 | 0.0 | 0.0 |
| Thyroid Cancer A302152 | 1.4 | 0.1 | 0.4 | 0.0 |
| Thyroid Margin A302153 | 1.7 | 0.9 | 6.4 | 4.1 |
| Normal Breast | 20.3 | 5.7 | 13.0 | 20.9 |
| Breast Cancer (OD04566) | 0.3 | 0.1 | 0.5 | 0.3 |
| Breast Cancer (OD04590-01) | 2.4 | 2.2 | 1.1 | 1.9 |
| Breast Cancer Mets (OD04590-03) | 0.7 | 0.1 | 0.3 | 0.0 |
| Breast Cancer Metastasis | 6.9 | 3.3 | 7.7 | 9.3 |
| (OD04655-05) | | | | |
| Breast Cancer 064006 | 5.6 | 8.3 | 4.2 | 4.9 |
| Breast Cancer 1024 | 47.0 | 19.3 | 30.1 | 23.5 |
| Breast Cancer 9100266 | 14.9 | 9.3 | 15.7 | 21.8 |
| Breast Margin 9100265 | 4.6 | 1.5 | 4.4 | 8.4 |
| Breast Cancer A209073 | 48.3 | 12.9 | 28.3 | 40.9 |
| Breast Margin A209073 | 38.7 | 16.6 | 27.5 | 29.7 |
| Normal Liver | 0.2 | 0.1 | 0.0 | 0.2 |
| Liver Cancer 064003 | 11.8 | 5.6 | 4.6 | 4.5 |
| Liver Cancer 1025 | 4.5 | 1.6 | 4.2 | 2.8 |
| Liver Cancer 1026 | 6.2 | 6.7 | 9.0 | 6.4 |
| Liver Cancer 6004-T | 15.6 | 3.3 | 3.5 | 2.8 |
| Liver Tissue 6004-N | 0.1 | 0.2 | 0.2 | 0.4 |
| Liver Cancer 6005-T | 14.6 | 8.0 | 8.7 | 7.9 |
| Liver Tissue 6005-N | 6.4 | 7.0 | 5.9 | 3.2 |
| Normal Bladder | 1.3 | 0.9 | 1.6 | 0.9 |
| Bladder Cancer 1023 | 0.4 | 0.2 | 0.3 | 0.3 |
| Bladder Cancer A302173 | 7.5 | 5.3 | 12.1 | 19.8 |
| Bladder Cancer (OD04718-01) | 27.7 | 23.0 | 35.6 | 41.5 |
| Bladder Normal Adjacent | 0.4 | 0.3 | 0.6 | 0.7 |
| (OD04718-03) | | | | |
| Normal Ovary | 1.4 | 0.4 | 1.1 | 0.3 |
| Ovarian Cancer 064008 | 1.3 | 0.5 | 1.3 | 1.3 |
| Ovarian Cancer (OD04768-07) | 0.4 | 0.2 | 0.0 | 0.0 |
| Ovary Margin (OD04768-08) | 1.0 | 0.8 | 0.6 | 1.6 |
| Normal Stomach | 4.3 | 2.0 | 0.3 | 3.1 |
| Gastric Cancer 9060358 | 2.4 | 0.7 | 0.7 | 1.2 |
| Stomach Margin 9060359 | 0.0 | 0.0 | 0.7 | 4.3 |
| Gastric Cancer 9060395 | 0.9 | 2.0 | 1.9 | 2.4 |
| Stomach Margin 9060394 | 0.2 | 0.3 | 1.1 | 0.2 |
| Gastric Cancer 9060397 | 0.4 | 0.5 | 1.6 | 0.7 |
| Stomach Margin 9060396 | 0.0 | 0.1 | 0.2 | 0.2 |
| Gastric Cancer 064005 | 0.7 | 0.7 | 0.9 | 1.5 |

Column A - Rel. Exp. (%) Ag047, Run 144771648
Column B - Rel. Exp. (%) Ag047, Run 152940364
Column C - Rel. Exp. (%) Ag2679, Run 158633803
Column D - Rel. Exp. (%) Ag2728, Run 158561830

TABLE FI
Panel 3D

| Tissue Name | A |
|---|---|
| Daoy- Medulloblastoma | 0.9 |
| TE671- Medulloblastoma | 0.0 |
| D283 Med- Medulloblastoma | 0.0 |
| PFSK-1- Primitive Neuroectodermal | 0.0 |
| XF-498- CNS | 0.0 |
| SNB-78- Glioma | 100.0 |
| SF-268- Glioblastoma | 79.0 |
| T98G- Glioblastoma | 1.4 |
| SK-N-SH- Neuroblastoma (metastasis) | 2.0 |
| SF-295- Glioblastoma | 0.1 |
| Cerebellum | 4.5 |
| Cerebellum | 2.9 |
| NCI-H292- Mucoepidermoid lung carcinoma | 3.8 |
| DMS-114- Small cell lung cancer | 0.0 |
| DMS-79- Small cell lung cancer | 0.1 |
| NCI-H146- Small cell lung cancer | 0.0 |
| NCI-H526- Small cell lung cancer | 1.0 |
| NCI-N417- Small cell lung cancer | 0.0 |
| NCI-H82- Small cell lung cancer | 7.6 |
| NCI-H157- Squamous cell lung cancer (metastasis) | 0.8 |
| NCI-H1155- Large cell lung cancer | 0.0 |
| NCI-H1299- Large cell lung cancer | 0.2 |
| NCI-H727- Lung carcinoid | 0.0 |
| NCI-UMC-11- Lung carcinoid | 0.0 |
| LX-1- Small cell lung cancer | 0.0 |
| Colo-205- Colon cancer | 0.0 |
| KM12- Colon cancer | 0.0 |
| KM20L2- Colon cancer | 0.0 |
| NCI-H716- Colon cancer | 0.0 |
| SW-48- Colon adenocarcinoma | 0.0 |
| SW1116- Colon adenocarcinoma | 0.0 |
| LS 174T- Colon adenocarcinoma | 0.0 |
| SW-948- Colon adenocarcinoma | 0.0 |
| SW-480- Colon adenocarcinoma | 0.0 |
| NCI-SNU-5- Gastric carcinoma | 0.7 |
| KATO III- Gastric carcinoma | 0.0 |
| NCI-SNU-16- Gastric carcinoma | 0.2 |
| NCI-SNU-1- Gastric carcinoma | 0.0 |
| RF-1- Gastric adenocarcinoma | 0.3 |
| RF-48- Gastric adenocarcinoma | 0.1 |
| MKN-45- Gastric carcinoma | 0.0 |
| NCI-N87- Gastric carcinoma | 0.0 |
| OVCAR-5- Ovarian carcinoma | 0.0 |
| RL95-2- Uterine carcinoma | 4.0 |
| HelaS3- Cervical adenocarcinoma | 0.0 |
| Ca Ski- Cervical epidermoid carcinoma (metastasis) | 0.3 |
| ES-2- Ovarian clear cell carcinoma | 6.7 |
| Ramos- Stimulated with PMA/ionomycin 6 h | 0.0 |
| Ramos- Stimulated with PMA/ionomycin 14 h | 0.0 |
| MEG-01- Chronic myelogenous leukemia | 0.0 |
| (megokaryoblast) | |
| Raji- Burkitt's lymphoma | 0.0 |
| Daudi- Burkitt's lymphoma | 0.0 |
| U266- B-cell plasmacytoma | 0.0 |
| CA46- Burkitt's lymphoma | 0.0 |
| RL- non-Hodgkin's B-cell lymphoma | 0.0 |
| JM1- pre-B-cell lymphoma | 0.0 |
| Jurkat- T cell leukemia | 0.0 |
| TF-1- Erythroleukemia | 0.0 |
| HUT 78- T-cell lymphoma | 0.0 |
| U937- Histiocytic lymphoma | 0.0 |
| KU-812- Myelogenous leukemia | 0.0 |
| 769-P- Clear cell renal carcinoma | 0.1 |
| Caki-2- Clear cell renal carcinoma | 0.1 |
| SW 839- Clear cell renal carcinoma | 0.0 |
| Rhabdoid kidney tumor | 6.0 |
| Hs766T- Pancreatic carcinoma (LN metastasis) | 0.0 |
| CAPAN-1- Pancreatic adenocarcinoma (liver metastasis) | 0.0 |
| SU86.86- Pancreatic carcinoma (liver metastasis) | 0.1 |
| BxPC-3- Pancreatic adenocarcinoma | 0.6 |
| HPAC- Pancreatic adenocarcinoma | 0.0 |
| MIA PaCa-2- Pancreatic carcinoma | 0.0 |
| CFPAC-1- Pancreatic ductal adenocarcinoma | 0.1 |
| PANC-1- Pancreatic epithelioid ductal carcinoma | 0.0 |
| T24- Bladder carcinma (transitional cell) | 0.0 |
| 5637- Bladder carcinoma | 3.8 |
| HT-1197- Bladder carcinoma | 0.0 |
| UM-UC-3- Bladder carcinma (transitional cell) | 10.3 |
| A204- Rhabdomyosarcoma | 0.0 |
| HT-1080- Fibrosarcoma | 59.9 |
| MG-63- Osteosarcoma | 0.5 |
| SK-LMS-1- Leiomyosarcoma (vulva) | 4.2 |
| SJRH30- Rhabdomyosarcoma (met to bone marrow) | 0.3 |
| A431- Epidermoid carcinoma | 0.6 |
| WM266-4- Melanoma | 0.0 |
| DU 145- Prostate carcinoma (brain metastasis) | 0.0 |
| MDA-MB-468- Breast adenocarcinoma | 0.0 |
| SCC-4- Squamous cell carcinoma of tongue | 0.2 |
| SCC-9- Squamous cell carcinoma of tongue | 1.1 |
| SCC-15- Squamous cell carcinoma of tongue | 0.3 |
| CAL 27- Squamous cell carcinoma of tongue | 7.0 |

Column A - Rel. Exp. (%) Ag047, Run 158634002

TABLE FJ
Panel 4.1D

| Tissue Name | A | B | C |
|---|---|---|---|
| Secondary Th1 act | 0.0 | 0.0 | 0.0 |
| Secondary Th2 act | 0.0 | 0.0 | 0.1 |
| Secondary Tr1 act | 0.0 | 0.0 | 0.0 |
| Secondary Th1 rest | 0.0 | 0.0 | 0.0 |
| Secondary Th2 rest | 0.0 | 0.0 | 0.2 |
| Secondary Tr1 rest | 0.0 | 0.0 | 0.0 |
| Primary Th1 act | 0.0 | 0.0 | 0.1 |
| Primary Th2 act | 0.0 | 0.0 | 0.0 |
| Primary Tr1 act | 0.0 | 0.0 | 0.0 |
| Primary Th1 rest | 0.0 | 0.0 | 0.0 |
| Primary Th2 rest | 0.0 | 0.0 | 0.0 |
| Primary Tr1 rest | 0.0 | 0.0 | 0.0 |
| CD45RA CD4 lymphocyte act | 0.6 | 0.8 | 1.7 |
| CD45RO CD4 lymphocyte act | 0.2 | 0.0 | 0.2 |
| CD8 lymphocyte act | 0.0 | 0.0 | 0.0 |
| Secondary CD8 lymphocyte rest | 0.1 | 0.1 | 0.0 |
| Secondary CD8 lymphocyte act | 0.0 | 0.0 | 0.0 |
| CD4 lymphocyte none | 0.0 | 0.0 | 0.2 |
| 2ry Th1/Th2/Tr1_anti-CD95 CH11 | 0.0 | 0.0 | 0.0 |
| LAK cells rest | 0.0 | 0.0 | 0.0 |
| LAK cells IL-2 | 0.0 | 0.0 | 0.1 |
| LAK cells IL-2 + IL-12 | 0.0 | 0.0 | 0.0 |
| LAK cells IL-2 + IFN gamma | 0.0 | 0.0 | 0.0 |
| LAK cells IL-2 + IL-18 | 0.0 | 0.1 | 0.2 |
| LAK cells PMA/ionomycin | 0.0 | 0.0 | 0.0 |
| NK Cells IL-2 rest | 0.0 | 0.0 | 0.0 |
| Two Way MLR 3 day | 0.0 | 0.0 | 0.0 |
| Two Way MLR 5 day | 0.0 | 0.0 | 0.1 |
| Two Way MLR 7 day | 0.0 | 0.1 | 0.0 |
| PBMC rest | 0.0 | 0.0 | 0.0 |
| PBMC PWM | 0.0 | 0.0 | 0.0 |
| PBMC PHA-L | 0.0 | 0.0 | 0.0 |
| Ramos (B cell) none | 0.0 | 0.0 | 0.0 |
| Ramos (B cell) ionomycin | 0.0 | 0.0 | 0.0 |
| B lymphocytes PWM | 0.0 | 0.0 | 0.0 |
| B lymphocytes CD40L and IL-4 | 0.0 | 0.0 | 0.0 |
| EOL-1 dbcAMP | 0.0 | 0.0 | 0.0 |
| EOL-1 dbcAMP PMA/ionomycin | 0.0 | 0.0 | 0.0 |
| Dendritic cells none | 0.0 | 0.0 | 0.0 |
| Dendritic cells LPS | 0.0 | 0.0 | 0.0 |
| Dendritic cells anti-CD40 | 0.0 | 0.1 | 0.3 |
| Monocytes rest | 0.0 | 0.0 | 0.0 |
| Monocytes LPS | 0.0 | 0.0 | 0.2 |
| Macrophages rest | 0.0 | 0.0 | 0.0 |
| Macrophages LPS | 0.0 | 0.0 | 0.0 |
| HUVEC none | 0.0 | 0.0 | 0.3 |
| HUVEC starved | 0.0 | 0.4 | 0.2 |
| HUVEC IL-1beta | 0.0 | 0.0 | 0.0 |
| HUVEC IFN gamma | 0.0 | 0.0 | 0.0 |
| HUVEC TNF alpha + IFN gamma | 0.0 | 0.0 | 0.2 |
| HUVEC TNF alpha + IL4 | 0.0 | 0.0 | 0.0 |
| HUVEC IL-11 | 0.0 | 0.0 | 0.2 |
| Lung Microvascular EC none | 0.0 | 0.0 | 0.0 |
| Lung Microvascular EC TNFalpha + | 0.0 | 1.1 | 0.0 |
| IL-1beta | | | |
| Microvascular Dermal EC none | 0.0 | 0.0 | 0.0 |
| Microsvasular Dermal EC | 0.0 | 0.0 | 0.0 |
| TNFalpha + IL-1beta | | | |
| Bronchial epithelium TNFalpha + | 17.3 | 37.4 | 28.5 |
| IL1beta | | | |
| Small airway epithelium none | 40.3 | 47.6 | 49.0 |
| Small airway epithelium TNFalpha + | 100.0 | 100.0 | 100.0 |
| IL-1beta | | | |
| Coronery artery SMC rest | 0.7 | 0.8 | 0.4 |
| Coronery artery SMC TNFalpha + IL- | 0.5 | 0.6 | 1.3 |
| 1beta | | | |
| Astrocytes rest | 26.1 | 51.8 | 24.5 |
| Astrocytes TNFalpha + IL-1 beta | 24.1 | 78.5 | 42.6 |
| KU-812 (Basophil) rest | 0.0 | 0.0 | 0.0 |
| KU-812 (Basophil) PMA/ionomycin | 0.0 | 0.0 | 0.0 |
| CCD1106 (Keratinocytes) none | 52.5 | 45.1 | 90.1 |
| CCD1106 (Keratinocytes) | 14.4 | 33.7 | 20.9 |
| TNFalpha + IL-1 beta | | | |
| Liver cirrhosis | 0.3 | 0.2 | 0.2 |
| NCI-H292 none | 0.4 | 0.5 | 0.7 |
| NCI-H292 IL-4 | 9.9 | 12.6 | 19.1 |
| NCI-H292 IL-9 | 0.8 | 0.6 | 1.6 |
| NCI-H292 IL-13 | 14.0 | 10.9 | 13.2 |
| NCI-H292 IFN gamma | 0.1 | 0.9 | 0.4 |
| HPAEC none | 0.0 | 1.2 | 0.4 |
| HPAEC TNF alpha + IL-1 beta | 0.0 | 0.3 | 0.0 |
| Lung fibroblast none | 0.7 | 1.5 | 1.6 |
| Lung fibroblast TNF alpha + IL-1 | 0.2 | 0.0 | 0.7 |
| beta | | | |
| Lung fibroblast IL-4 | 0.1 | 0.1 | 0.0 |
| Lung fibroblast IL-9 | 0.2 | 0.2 | 0.1 |
| Lung fibroblast IL-13 | 0.2 | 1.3 | 0.4 |
| Lung fibroblast IFN gamma | 0.0 | 0.8 | 0.0 |
| Dermal fibroblast CCD1070 rest | 4.8 | 4.1 | 5.7 |
| Dermal fibroblast CCD1070 TNF | 1.3 | 2.2 | 2.1 |
| alpha | | | |
| Dermal fibroblast CCD1070 IL-1 beta | 1.1 | 0.8 | 1.8 |
| Dermal fibroblast IFN gamma | 3.1 | 2.4 | 3.4 |
| Dermal fibroblast IL-4 | 3.9 | 5.4 | 6.0 |
| Dermal Fibroblasts rest | 7.4 | 9.3 | 9.3 |
| Neutrophils TNFa + LPS | 0.0 | 0.6 | 0.0 |
| Neutrophils rest | 0.0 | 0.0 | 0.0 |
| Colon | 0.0 | 0.5 | 0.0 |
| Lung | 0.1 | 1.6 | 0.1 |
| Thymus | 1.6 | 9.3 | 2.3 |
| Kidney | 1.5 | 11.3 | 1.7 |

Column A - Rel. Exp. (%) Ag047, Run 264524965
Column B - Rel. Exp. (%) Ag2975, Run 171818669
Column C - Rel. Exp. (%) Ag47b, Run 263594820

Panel 1 Summary: Ag047/Ag332/Ag47b Highest expression of this gene was detected in a brain cancer cell line (CTs=23-25). There was also significant expression in clusters of samples from melanoma, ovarian cancer, breast, lung, renal colon and brain cancer lines. Expression of this gene is associated with these forms of cancer and is useful as a diagnostic marker for the presence of these cancers. Therapeutic modulation of the expression or function of these genes or gene products is useful in the treatment of ovarian, breast, lung, renal, and brain cancer and melanoma.

In addition to significant expression in brain cancer cell lines, this gene was preferentially expressed in the brain. This expression profile shows that this gene product plays a role in CNS processes. This gene encodes a homolog of a member of the neurestin farnily, Ten M2, and plays a role in neuronal regeneration. (Otaki, J. Dev Biol 1999 August 1;212(1):165-81 ) Agents that induce the expression or activity of this gene or gene product have utility as neuronal regeneration drugs and specifically for the treatment of neurodegenerative diseases, stroke, and neuronal trauma.

Among tissues with metabolic function, this gene showed consistent expression in thyroid, adult and fetal heart, liver and skeletal muscle. Thus, this gene product is an antibody target for the treatment of metabolic and endocrine disease, including obesity and Types 1 and 2 diabetes. In addition, this gene was eressed at higher levels in adult liver than in fetal liver and is be useful for differentiating between the two sources of liver tissue.

Panel 2D Summary: Ag047/Ag2679/Ag2728 Highest expression of this gene was detected in lung cancer samples (CTs=27-29). This gene was expressed at a higher level in gastric, bladder, and two samples each of lung and kidney cancer relative to the normal adjacent tissues. Expression of this gene is useful as a marker to detect the presence of these cancers. Therapeutic modulation of this gene, expressed protein and/or use of antibodies, protein therapeutics or small molecule drugs are useful in the treatment of these cancers.

Panel 3D Summary: Ag047 This gene was expressed in squamous cell carcinoma, glioma, small cell lung cancer cell lines. Expression of this gene is associated with these cancers and therapeutic modulation of this gene expressed protein and/or use of antibodies, protein therapeutics or small molecule drugs is of use in the treatment of these cancers.

Panel 4.1D Summary: Ag047/Ag047b/Ag2975 Highest expression of this gene was detected in activated small airway epithelium (CT=27-32). This gene was expressed at moderate level in small airway epithelium, astrocytes and keratinocytes. The expression of this gene in these tissues was up-regulated upon treatment with the inflammatory cytokines TNF-a and IL1. This gene encodes for a neurestin like molecule whose role in neuronal regeneration has been demonstrated. Modulation of the expression of this gene or its expressed protein is useful in the regeneration or repair mechanism of these tissues during inflammation and for the treatment of inflammatory brain and lung diseases such as bronchitis, chronic pulmonary diseases, skin and CNS inflammatory diseases, multiple sclerosis or stroke.

G. CG50646-04: Polydom Protein

Expression of gene CG50646-04 was assessed using the primer-probe sets Ag768 and Ag984, described in Tables GA and GB. Results of the RTQ-PCR runs are shown in Tables GC and GD.

TABLE GA
Probe Name Ag768

| | | | | |
|---|---|---|---|---|
| Primers | Sequences | Length | Start Position | SEQ ID No |

| | | | | |
|---|---|---|---|---|
| Forward | 5′-gggctataagtcagtcggaagt-3′ | 22 | 6772 | 1258 |
| Probe | TET-5′-cctgtatttgtctgccaagccaatcg-3′-TAMRA | 26 | 6794 | 1259 |
| Reverse | 5′-acagtcgagaggaacacacatc-3′ | 22 | 6844 | 1260 |

TABLE GB
Probe Name Ag984

| | | | | SEQ |
|---|---|---|---|---|
| | | | Start | ID |
| Primers | Sequences | Length | Position | No |
| Forward | 5′-cccgggttacactgatacct-3′ | 20 | 6755 | 1261 |
| Probe | TET-5′-tgctccagaaagccattctcaacctt-3′- | 26 | 6698 | 1262 |
| | TAMRA | | | |
| Reverse | 5′-cggtatcttgtggtgaacca-3′ | 20 | 6675 | 1263 |

TABLE GC
Panel 1.2

| Tissue Name | A |
|---|---|
| Endothelial cells | 0.0 |
| Heart (Fetal) | 0.0 |
| Pancreas | 0.0 |
| Pancreatic ca. CAPAN 2 | 0.0 |
| Adrenal Gland | 0.1 |
| Thyroid | 0.0 |
| Salivary gland | 0.0 |
| Pituitary gland | 0.0 |
| Brain (fetal) | 0.0 |
| Brain (whole) | 0.0 |
| Brain (amygdala) | 0.0 |
| Brain (cerebellum) | 0.0 |
| Brain (hippocampus) | 0.0 |
| Brain (thalamus) | 0.0 |
| Cerebral Cortex | 0.0 |
| Spinal cord | 0.0 |
| glio/astro U87-MG | 0.0 |
| glio/astro U-118-MG | 0.0 |
| astrocytoma SW1783 | 0.0 |
| neuro*; met SK-N-AS | 0.0 |
| astrocytoma SF-539 | 0.0 |
| astrocytoma SNB-75 | 0.0 |
| glioma SNB-19 | 0.0 |
| glioma U251 | 0.0 |
| glioma SF-295 | 0.0 |
| Heart | 0.1 |
| Skeletal Muscle | 0.2 |
| Bone marrow | 0.0 |
| Thymus | 0.0 |
| Spleen | 0.0 |
| Lymph node | 0.7 |
| Colorectal Tissue | 0.0 |
| Stomach | 0.0 |
| Small intestine | 0.8 |
| Colon ca. SW480 | 0.0 |
| Colon ca.* SW620 (SW480 met) | 0.0 |
| Colon ca. HT29 | 0.0 |
| Colon ca. HCT-116 | 0.0 |
| Colon ca. CaCo-2 | 0.0 |
| Colon ca. Tissue (ODO3866) | 0.0 |
| Colon ca. HCC-2998 | 0.0 |
| Gastric ca.* (liver met) NCI-N87 | 0.0 |
| Bladder | 0.0 |
| Trachea | 0.0 |
| Kidney | 0.0 |
| Kidney (fetal) | 0.0 |
| Renal ca. 786-0 | 0.0 |
| Renal ca. A498 | 0.0 |
| Renal ca. RXF 393 | 0.0 |
| Renal ca. ACHN | 0.0 |
| Renal ca. UO-31 | 0.0 |
| Renal ca. TK-10 | 0.0 |
| Liver | 0.0 |
| Liver (fetal) | 0.0 |
| Liver ca. (hepatoblast) HepG2 | 0.0 |
| Lung | 0.6 |
| Lung (fetal) | 0.0 |
| Lung ca. (small cell) LX-1 | 0.0 |
| Lung ca. (small cell) NCI-H69 | 0.0 |
| Lung ca. (s. cell var.) SHP-77 | 0.0 |
| Lung ca. (large cell)NCI-H460 | 0.0 |
| Lung ca. (non-sm. cell) A549 | 0.0 |
| Lung ca. (non-s. cell) NCI-H23 | 0.0 |
| Lung ca. (non-s. cell) HOP-62 | 0.0 |
| Lung ca. (non-s. cl) NCI-H522 | 0.0 |
| Lung ca. (squam.) SW 900 | 0.0 |
| Lung ca. (squam.) NCI-H596 | 0.0 |
| Mammary gland | 4.4 |
| Breast ca.* (pl. ef) MCF-7 | 0.0 |
| Breast ca.* (pl. ef) MDA-MB-231 | 0.0 |
| Breast ca.* (pl. ef) T47D | 0.0 |
| Breast ca. BT-549 | 0.0 |
| Breast ca. MDA-N | 0.0 |
| Ovary | 0.0 |
| Ovarian ca. OVCAR-3 | 0.0 |
| Ovarian ca. OVCAR-4 | 0.0 |
| Ovarian ca. OVCAR-5 | 0.0 |
| Ovarian ca. OVCAR-8 | 0.0 |
| Ovarian ca. IGROV-1 | 0.0 |
| Ovarian ca. (ascites) SK-OV-3 | 0.0 |
| Uterus | 0.2 |
| Placenta | 100.0 |
| Prostate | 0.0 |
| Prostate ca.* (bone met) PC-3 | 0.0 |
| Testis | 0.0 |
| Melanoma Hs688(A).T | 0.0 |
| Melanoma* (met) Hs688(B).T | 0.0 |
| Melanoma UACC-62 | 0.0 |
| Melanoma M14 | 0.0 |
| Melanoma LOX IMVI | 0.0 |
| Melanoma* (met) SK-MEL-5 | 0.0 |

Column A - Rel. Exp. (%) Ag768, Run 116422776

TABLE GD
Panel 4D

| Tissue Name | A |
|---|---|
| Secondary Th1 act | 0.0 |
| Secondary Th2 act | 0.1 |
| Secondary Tr1 act | 0.0 |
| Secondary Th1 rest | 0.1 |
| Secondary Th2 rest | 0.1 |
| Secondary Tr1 rest | 0.2 |
| Primary Th1 act | 0.0 |
| Primary Th2 act | 0.0 |
| Primary Tr1 act | 0.0 |
| Primary Th1 rest | 0.2 |
| Primary Th2 rest | 0.0 |
| Primary Tr1 rest | 0.0 |
| CD45RA CD4 lymphocyte act | 6.4 |
| CD45RO CD4 lymphocyte act | 0.1 |
| CD8 lymphocyte act | 0.0 |
| Secondary CD8 lymphocyte rest | 0.1 |
| Secondary CD8 lymphocyte act | 0.1 |
| CD4 lymphocyte none | 0.0 |
| 2ry Th1/Th2/Tr1_anti-CD95 CH11 | 0.0 |
| LAK cells rest | 0.2 |
| LAK cells IL-2 | 0.0 |
| LAK cells IL-2 + IL-12 | 0.1 |
| LAK cells IL-2 + IFN gamma | 0.3 |
| LAK cells IL-2 + IL-18 | 0.2 |
| LAK cells PMA/ionomycin | 3.5 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 2.5 |
| PBMC PHA-L | 0.3 |
| Ramos (B cell) none | 0.0 |
| Ramos (B cell) ionomycin | 0.1 |
| B lymphocytes PWM | 0.1 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.2 |
| Dendritic cells anti-CD40 | 0.1 |
| Monocytes rest | 0.1 |
| Monocytes LPS | 3.1 |
| Macrophages rest | 0.1 |
| Macrophages LPS | 3.3 |
| HUVEC none | 0.0 |
| HUVEC starved | 0.2 |
| HUVEC IL-1beta | 0.2 |
| HUVEC IFN gamma | 0.3 |
| HUVEC TNF alpha + IFN gamma | 0.1 |
| HUVEC TNF alpha + IL4 | 0.1 |
| HUVEC IL-11 | 0.1 |
| Lung Microvascular EC none | 0.0 |
| Lung Microvascular EC TNFalpha + IL-1beta | 0.0 |
| Microvascular Dermal EC none | 0.1 |
| Microsvasular Dermal EC TNFalpha + IL-1beta | 0.2 |
| Bronchial epithelium TNFalpha + IL1beta | 0.2 |
| Small airway epithelium none | 2.3 |
| Small airway epithelium TNFalpha + IL-1beta | 1.3 |
| Coronery artery SMC rest | 15.1 |
| Coronery artery SMC TNFalpha + IL-1beta | 8.2 |
| Astrocytes rest | 1.8 |
| Astrocytes TNFalpha + IL-1beta | 2.4 |
| KU-812 (Basophil) rest | 0.0 |
| KU-812 (Basophil) PMA/ionomycin | 0.0 |
| CCD1106 (Keratinocytes) none | 0.4 |
| CCD1106 (Keratinocytes) TNFalpha + IL-1beta | 1.4 |
| Liver cirrhosis | 10.6 |
| Lupus kidney | 7.0 |
| NCI-H292 none | 24.1 |
| NCI-H292 IL-4 | 21.6 |
| NCI-H292 IL-9 | 30.4 |
| NCI-H292 IL-13 | 15.3 |
| NCI-H292 IFN gamma | 14.6 |
| HPAEC none | 0.4 |
| HPAEC TNF alpha + IL-1 beta | 0.0 |
| Lung fibroblast none | 31.0 |
| Lung fibroblast TNF alpha + IL-1 beta | 7.7 |
| Lung fibroblast IL-4 | 55.5 |
| Lung fibroblast IL-9 | 37.4 |
| Lung fibroblast IL-13 | 86.5 |
| Lung fibroblast IFN gamma | 100.0 |
| Dermal fibroblast CCD1070 rest | 28.9 |
| Dermal fibroblast CCD1070 TNF alpha | 23.7 |
| Dermal fibroblast CCD1070 IL-1 beta | 20.2 |
| Dermal fibroblast IFN gamma | 22.5 |
| Dermal fibroblast IL-4 | 47.0 |
| IBD Colitis 2 | 0.5 |
| IBD Crohn's | 2.0 |
| Colon | 9.2 |
| Lung | 20.3 |
| Thymus | 13.0 |
| Kidney | 6.4 |

Column A - Rel. Exp. (%) Ag768, Run 138175130

Panel 1.2 Summary: Ag768 Highest expression of this gene was seen in placenta (CT=21). This gene encodes a polydom-like protein and was also highly expressed in mammary gland, skeletal muscle. This gene may be involved in cellular adhesion (Gilges D. Biochem J 2000 November 15;352 Pt 1:49-59). Expression of this gene is useful in differentiating between placental tissues and other tissues on this panel. Modulation of this gene or its protein product is useful in reproductive and skeletal muscle physiology.

This gene was more highly expressed in fetal kidney (CT=33) than in adult kidney (CT=40). Conversely, this gene was more highly expressed in adult lung and liver (CTs=28-32) than in fetal lung and liver (CTs=38-40). Thus, expression of this gene is useful in differentiating between the adult and fetal sources of these tissues.

Panel 4D Summary: Ag768 Highest expression of this gene was seen in lung fibroblasts stimulated with IFN-gamma (CT=27.4). Significant expression was seen in many samples derived from the lung including lung fibroblasts stimulated with different cytokines, the pulmonary mucoepidermoid cell line H292 stimulated with the same cytokines, and normal lung tissue. The expression of this gene in lung cells and lung tissue shows that this gene or its protein product is involved in normal homeostasis of the lung, as well as pathological and inflammatory lung disorders, including chronic obstructive pulmonary disease, asthma, allergy and emphysema.

Significant levels of expression of this gene in dermal fibroblasts show that this gene and its protein product is involved in skin disorders, including psoriasis.

Moderate to low expression of this gene was also seen in many other cells with important immune function, including stimulated macrophages and monocytes, coronary artery smooth muscle cells, stimulated peripheral blood mononuclear cells, lymphocyte activated killer cells (LAK), astrocytes, activated CD45RA cells, and normal colon, thymus and kidney. This widespread expression demonstrates that this gene or the protein encoded by this gene is involved in other inflammatory and autoimmune conditions, including inflammatory bowel disease, rheumatoid arthritis and osteoarthritis.

H. CG5073609: CD44-Like Precursor Fell

Expression of gene CG50736-09 was assessed using the primer-probe sets Ag2742, Ag2743, Ag2744, Ag2745, Ag2746, Ag793, Ag812, Ag03 and Ag068, described in Tables HA, HB, HC, HD, HE, HF, HG, HH and HI. Results of the RTQ-PCR runs are shown in Tables HJ, HK, HL, HM, HN and HO.

TABLE HA
Probe Name Ag2742

| | | | | SEQ |
|---|---|---|---|---|
| | | | Start | ID |
| Primers | Sequences | Length | Position | No |
| Forward | 5′-ctgcaaaatcttacgactttgg-3′ | 22 | 429 | 1264 |
| Probe | TET-5′-caacaaacaatggctacatcaaatttagca-3′- | 30 | 451 | 1265 |
| | TAMRA | | | |
| Reverse | 5′-atgacactcagcaaacctgagt-3′ | 22 | 493 | 1266 |

TABLE HB
Probe Name Ag2743

| | | | | SEQ |
|---|---|---|---|---|
| | | | Start | ID |
| Primers | Sequences | Length | Position | No |
| Forward | 5′-ctgcaaaatcttacgacttgg-3′ | 22 | 429 | 1267 |
| Probe | TET-5′-caacaaacaatggctacatcaaatttagca-3′- | 30 | 451 | 1268 |
| | TAMRA | | | |
| Reverse | 5′-atgacactcagcaaacctgagt-3′ | 22 | 493 | 1269 |

TABLE HC
Probe Name Ag2744

| | | | | SEQ |
|---|---|---|---|---|
| | | | Start | ID |
| Primers | Sequences | Length | Position | No |
| Forward | 5′-ctgcaaaatcttacgactttgg-3′ | 22 | 429 | 1270 |
| Probe | TET-5′-caacaaacaatggctacatcaaatttagca-3′- | 30 | 451 | 1271 |
| | TAMRA | | | |
| Reverse | 5′-tcagcaaacctgagtcctgta-3′ | 21 | 487 | 1272 |

TABLE HD
Probe Name Ag2745

| | | | | SEQ |
|---|---|---|---|---|
| | | | Start | ID |
| Primers | Sequences | Length | Position | No |
| Forward | 5′-ctgcaaaatcttacgactttgg-3′ | 22 | 429 | 1273 |
| Probe | TET-5′-caacaaacaatggctacatcaaatttagca-3′- | 30 | 451 | 1274 |
| | TAMRA | | | |
| Reverse | 5′-atgacactcagcaaacctgagt-3′ | 22 | 493 | 1275 |

TABLE HD
Probe Name Ag2746

| | | | | SEQ |
|---|---|---|---|---|
| | | | Start | ID |
| Primers | Sequences | Length | Position | No |
| Forward | 5′-ctgcaaaatcttacgactttgg-3′ | 22 | 429 | 1276 |
| Probe | TET-5′-caacaaacaatggctacatcaaatttagca-3′- | 30 | 451 | 1277 |
| | TAMRA | | | |
| Reverse | 5′-atgacactcagcaaacctgagt-3′ | 22 | 493 | 1278 |

TABLE HF
Probe Name Ag793

| | | | | SEQ |
|---|---|---|---|---|
| | | | Start | ID |
| Primers | Sequences | Length | Position | No |
| Forward | 5′-ccaaggttttagctgtggatct-3′ | 22 | 664 | 1279 |
| Probe | TET-5′-acatccactgcctggaagaccctg-3′- | 24 | 690 | 1280 |
| | TAMRA | | | |
| Reverse | 5′-cacatttcacactcagctctga-3′ | 22 | 720 | 1281 |

TABLE HG
Probe Name Ag812

| | | | | SEQ |
|---|---|---|---|---|
| | | | Start | ID |
| Primers | Sequences | Length | Position | No |
| Forward | 5′-caggagcatttcgtgaaaga-3′ | 20 | 57 | 1282 |
| Probe | TET-5′-ttttgcacctttatctgcagcctttg-3′- | 26 | 104 | 1283 |
| | TAMRA | | | |
| Reverse | 5′-tttaacccgagcttcctcat-3′ | 20 | 130 | 1284 |

TABLE HH
Probe Name Ag03

| | | | | SEQ |
|---|---|---|---|---|
| | | | Start | ID |
| Primers | Sequences | Length | Position | No |
| Forward | 5′-ctggttgtaggttgccatggt-3′ | 21 | 1884 | 1285 |
| Probe | TET-5′-cagcttcgttggcacaggcctctc-3′- | 24 | 1858 | 1286 |
| | TAMRA | | | |
| Reverse | 5′-ccagtataagctgacctttgacaaag-3′ | 26 | 1829 | 1287 |

TABLE HI
Probe Name Ag068

| | | | | SEQ |
|---|---|---|---|---|
| | | | Start | ID |
| Primers | Sequences | Length | Position | No |
| Forward | 5′-ctggttgtaggttgccatggt-3′ | 21 | 1884 | 1288 |
| Probe | TET-5′-cagcttcgttggcacaggcctctc-3′- | 24 | 1858 | 1289 |
| | TAMRA | | | |
| Reverse | 5′-ccagtataagctgacctttgacaaag-3′ | 26 | 1829 | 1290 |

TABLE HJ
Panel 1

| Tissue Name | A | B |
|---|---|---|
| Endothelial cells | 0.0 | 0.0 |
| Endothelial cells (treated) | 0.0 | 0.0 |
| Pancreas | 0.0 | 2.3 |
| Pancreatic ca. CAPAN 2 | 0.0 | 0.0 |
| Adrenal gland | 0.5 | 1.3 |
| Thyroid | 1.8 | 2.1 |
| Salivary gland | 2.5 | 3.0 |
| Pituitary gland | 0.8 | 0.8 |
| Brain (fetal) | 0.1 | 0.4 |
| Brain (whole) | 0.3 | 0.6 |
| Brain (amygdala) | 0.1 | 0.4 |
| Brain (cerebellum) | 0.4 | 0.7 |
| Brain (hippocampus) | 0.3 | 0.9 |
| Brain (substantia nigra) | 0.1 | 0.3 |
| Brain (thalamus) | 0.1 | 0.2 |
| Brain (hypothalamus) | 0.4 | 0.5 |
| Spinal cord | 0.0 | 0.3 |
| glio/astro U87-MG | 0.0 | 0.1 |
| glio/astro U-118-MG | 0.1 | 0.2 |
| astrocytoma SW1783 | 0.0 | 0.0 |
| neuro*; met SK-N-AS | 0.0 | 0.0 |
| astrocytoma SF-539 | 0.0 | 0.1 |
| astrocytoma SNB-75 | 0.1 | 0.2 |
| glioma SNB-19 | 0.6 | 1.6 |
| glioma U251 | 0.2 | 0.9 |
| glioma SF-295 | 0.1 | 0.2 |
| Heart | 0.3 | 0.3 |
| Skeletal muscle | 0.3 | 0.4 |
| Bone marrow | 3.5 | 3.0 |
| Thymus | 0.3 | 0.2 |
| Spleen | 100.0 | 100.0 |
| Lymph node | 29.3 | 81.2 |
| Colon (ascending) | 0.8 | 1.0 |
| Stomach | 1.2 | 1.5 |
| Small intestine | 1.6 | 1.7 |
| Colon ca. SW480 | 0.0 | 0.0 |
| Colon ca.* SW620 (SW480 met) | 0.0 | 0.1 |
| Colon ca. HT29 | 0.0 | 0.1 |
| Colon ca. HCT-116 | 0.0 | 0.0 |
| Colon ca. CaCo-2 | 0.1 | 0.1 |
| Colon ca. HCT-15 | 0.1 | 0.7 |
| Colon ca. HCC-2998 | 0.0 | 0.3 |
| Gastric ca. * (liver met) NCI-N87 | 0.1 | 0.2 |
| Bladder | 1.1 | 0.2 |
| Trachea | 2.4 | 2.2 |
| Kidney | 0.1 | 0.4 |
| Kidney (fetal) | 1.0 | 1.3 |
| Renal ca. 786-0 | 0.0 | 0.0 |
| Renal ca. A498 | 0.1 | 0.5 |
| Renal ca. RXF 393 | 0.0 | 0.0 |
| Renal ca. ACHN | 0.0 | 0.0 |
| Renal ca. UO-31 | 0.0 | 0.1 |
| Renal ca. TK-10 | 0.2 | 0.9 |
| Liver | 14.2 | 12.9 |
| Liver (fetal) | 25.7 | 15.6 |
| Liver ca. (hepatoblast) HepG2 | 0.0 | 0.1 |
| Lung | 0.5 | 0.1 |
| Lung (fetal) | 4.4 | 6.2 |
| Lung ca. (small cell) LX-1 | 0.0 | 0.0 |
| Lung ca. (small cell) NCI-H69 | 0.1 | 0.4 |
| Lung ca. (s. cell var.) SHP-77 | 0.0 | 0.0 |
| Lung ca. (large cell)NCI-H460 | 0.0 | 0.0 |
| Lung ca. (non-sm. cell) A549 | 0.0 | 0.1 |
| Lung ca. (non-s. cell) NCI-H23 | 0.0 | 0.3 |
| Lung ca. (non-s. cell) HOP-62 | 0.0 | 0.1 |
| Lung ca. (non-s. cl) NCI-H522 | 0.0 | 0.0 |
| Lung ca. (squam.) SW 900 | 0.0 | 0.1 |
| Lung ca. (squam.) NCI-H596 | 0.1 | 0.3 |
| Mammary gland | 4.7 | 5.1 |
| Breast ca.* (pl. ef) MCF-7 | 0.0 | 0.2 |
| Breast ca.* (pl. ef) MDA-MB-231 | 0.1 | 0.4 |
| Breast ca.* (pl. ef) T47D | 0.1 | 0.4 |
| Breast ca. BT-549 | 0.0 | 0.0 |
| Breast ca. MDA-N | 0.1 | 0.6 |
| Ovary | 3.6 | 1.3 |
| Ovarian ca. OVCAR-3 | 0.0 | 0.1 |
| Ovarian ca. OVCAR-4 | 0.0 | 0.0 |
| Ovarian ca. OVCAR-5 | 0.1 | 0.6 |
| Ovarian ca. OVCAR-8 | 0.2 | 0.7 |
| Ovarian ca. IGROV-1 | 0.0 | 0.3 |
| Ovarian ca. (ascites) SK-OV-3 | 0.0 | 0.2 |
| Uterus | 0.2 | 0.4 |
| Placenta | 1.9 | 1.6 |
| Prostate | 0.7 | 1.0 |
| Prostate ca.* (bone met) PC-3 | 0.0 | 0.0 |
| Testis | 23.5 | 22.1 |
| Melanoma Hs688(A).T | 0.0 | 0.1 |
| Melanoma* (met) Hs688(B).T | 0.0 | 0.1 |
| Melanoma UACC-62 | 0.0 | 0.0 |
| Melanoma M14 | 0.1 | 0.5 |
| Melanoma LOX IMVI | 0.1 | 0.5 |
| Melanoma* (met) SK-MEL-5 | 0.0 | 0.0 |
| Melanoma SK-MEL-28 | 0.3 | 1.3 |

Column A - Rel. Exp. (%) Ag03, Run 87353672
Column B - Rel. Exp. (%) Ag068, Run 87361479

TABLE HK
Panel 1.2

| Tissue Name | A | B |
|---|---|---|
| Endothelial cells | 0.0 | 0.0 |
| Heart (Fetal) | 0.3 | 5.6 |
| Pancreas | 2.0 | 0.6 |
| Pancreatic ca. CAPAN 2 | 0.0 | 0.0 |
| Adrenal Gland | 0.3 | 0.9 |
| Thyroid | 2.0 | 0.7 |
| Salivary gland | 6.3 | 6.7 |
| Pituitary gland | 0.1 | 0.4 |
| Brain (fetal) | 0.1 | 0.0 |
| Brain (whole) | 1.2 | 0.1 |
| Brain (amygdala) | 0.0 | 0.1 |
| Brain (cerebellum) | 7.6 | 0.0 |
| Brain (hippocampus) | 0.1 | 0.2 |
| Brain (thalamus) | 0.0 | 0.0 |
| Cerebral Cortex | 0.1 | 0.1 |
| Spinal cord | 0.0 | 0.1 |
| glio/astro U87-MG | 0.0 | 0.0 |
| glio/astro U-118-MG | 0.0 | 0.0 |
| astrocytoma SW1783 | 0.0 | 0.0 |
| neuro*; met SK-N-AS | 0.0 | 0.0 |
| astrocytoma SF-539 | 0.0 | 0.1 |
| astrocytoma SNB-75 | 0.0 | 0.0 |
| glioma SNB-19 | 0.0 | 0.1 |
| glioma U251 | 0.0 | 0.1 |
| glioma SF-295 | 0.0 | 0.0 |
| Heart | 0.8 | 1.8 |
| Skeletal Muscle | 2.4 | 1.5 |
| Bone marrow | 2.7 | 3.5 |
| Thymus | 0.2 | 0.3 |
| Spleen | 44.8 | 44.4 |
| Lymph node | 39.2 | 51.8 |
| Colorectal Tissue | 0.0 | 0.2 |
| Stomach | 1.0 | 2.9 |
| Small intestine | 1.2 | 2.7 |
| Colon ca. SW480 | 0.0 | 0.0 |
| Colon ca.* SW620 (SW480 met) | 0.0 | 0.0 |
| Colon ca. HT29 | 0.0 | 0.1 |
| Colon ca. HCT-116 | 0.0 | 0.0 |
| Colon ca. CaCo-2 | 0.0 | 0.0 |
| Colon ca. Tissue (ODO3866) | 0.1 | 0.4 |
| Colon ca. HCC-2998 | 0.0 | 0.0 |
| Gastric ca.* (liver met) NCI-N87 | 0.0 | 0.1 |
| Bladder | 3.7 | 3.8 |
| Trachea | 1.1 | 1.9 |
| Kidney | 0.1 | 0.4 |
| Kidney (fetal) | 0.8 | 2.1 |
| Renal ca. 786-0 | 0.0 | 0.0 |
| Renal ca. A498 | 0.3 | 0.1 |
| Renal ca. RXF 393 | 0.0 | 0.0 |
| Renal ca. ACHN | 0.0 | 0.0 |
| Renal ca. UO-31 | 0.0 | 0.0 |
| Renal ca. TK-10 | 0.0 | 0.0 |
| Liver | 100.0 | 100.0 |
| Liver (fetal) | 37.9 | 58.2 |
| Liver ca. (hepatoblast) HepG2 | 0.0 | 0.0 |
| Lung | 0.4 | 0.8 |
| Lung (fetal) | 2.4 | 2.5 |
| Lung ca. (small cell) LX-1 | 0.0 | 0.0 |
| Lung ca. (small cell) NCI-H69 | 0.1 | 0.6 |
| Lung ca. (s. cell var.) SHP-77 | 0.0 | 0.0 |
| Lung ca. (large cell) NCI-H460 | 0.0 | 0.2 |
| Lung ca. (non-sm. cell) A549 | 0.1 | 0.2 |
| Lung ca. (non-s. cell) NCI-H23 | 0.0 | 0.1 |
| Lung ca. (non-s. cell) HOP-62 | 0.0 | 0.0 |
| Lung ca. (non-s. cl) NCI-H522 | 0.0 | 0.0 |
| Lung ca. (squam.) SW 900 | 0.0 | 0.0 |
| Lung ca. (squam.) NCI-H596 | 0.1 | 0.2 |
| Mammary gland | 2.9 | 2.3 |
| Breast ca.* (pl. ef) MCF-7 | 0.0 | 0.0 |
| Breast ca.* (pl. ef) MDA-MB-231 | 0.0 | 0.0 |
| Breast ca.* (pl. ef) T47D | 0.1 | 0.3 |
| Breast ca. BT-549 | 0.0 | 0.0 |
| Breast ca. MDA-N | 0.0 | 0.1 |
| Ovary | 1.4 | 4.2 |
| Ovarian ca. OVCAR-3 | 2.9 | 0.0 |
| Ovarian ca. OVCAR-4 | 4.1 | 0.0 |
| Ovarian ca. OVCAR-5 | 0.2 | 0.3 |
| Ovarian ca. OVCAR-8 | 0.0 | 0.1 |
| Ovarian ca. IGROV-1 | 0.0 | 0.0 |
| Ovarian ca. (ascites) SK-OV-3 | 0.0 | 0.0 |
| Uterus | 0.2 | 0.7 |
| Placenta | 0.8 | 0.9 |
| Prostate | 0.3 | 0.5 |
| Prostate ca.* (bone met) PC-3 | 0.0 | 0.0 |
| Testis | 12.2 | 8.4 |
| Melanoma Hs688(A).T | 0.0 | 0.0 |
| Melanoma* (met) Hs688(B).T | 0.0 | 0.1 |
| Melanoma UACC-62 | 0.0 | 0.0 |
| Melanoma M14 | 0.1 | 0.2 |
| Melanoma LOX IMVI | 0.0 | 0.0 |
| Melanoma* (met) SK-MEL-5 | 0.1 | 0.0 |

Column A - Rel. Exp. (%) Ag812, Run 118348259
Column B - Rel. Exp. (%) Ag812, Run 121953945

TABLE HL
Panel 1.3D

| Tissue Name | A | B | C | D | E |
|---|---|---|---|---|---|
| Liver adenocarcinoma | 0.0 | 0.0 | 0.0 | 0.0 | 0.0 |
| Pancreas | 0.2 | 0.4 | 0.3 | 0.2 | 0.2 |
| Pancreatic ca. CAPAN 2 | 0.0 | 0.0 | 0.0 | 0.0 | 0.0 |
| Adrenal gland | 0.2 | 0.0 | 0.0 | 0.4 | 0.2 |
| Thyroid | 0.5 | 0.6 | 1.1 | 1.4 | 0.6 |
| Salivary gland | 1.0 | 1.0 | 0.7 | 0.7 | 0.1 |
| Pituitary gland | 0.0 | 0.0 | 0.1 | 0.0 | 0.0 |
| Brain (fetal) | 0.0 | 0.0 | 0.0 | 0.0 | 0.0 |
| Brain (whole) | 0.0 | 0.0 | 0.1 | 0.0 | 0.0 |
| Brain (amygdala) | 0.0 | 0.3 | 0.1 | 0.0 | 0.0 |
| Brain (cerebellum) | 0.0 | 0.0 | 0.0 | 0.0 | 0.0 |
| Brain (hippocampus) | 0.3 | 0.0 | 0.1 | 0.0 | 0.0 |
| Brain (substantia nigra) | 0.0 | 0.0 | 0.0 | 0.0 | 0.0 |
| Brain (thalamus) | 0.0 | 0.0 | 0.0 | 0.0 | 0.0 |
| Cerebral Cortex | 0.0 | 0.0 | 0.0 | 0.1 | 0.0 |
| Spinal cord | 0.0 | 0.0 | 0.0 | 0.0 | 0.0 |
| glio/astro U87-MG | 0.0 | 0.0 | 0.0 | 0.0 | 0.0 |
| glio/astro U-118-MG | 0.0 | 0.0 | 0.0 | 0.0 | 0.0 |
| astrocytoma SW1783 | 0.0 | 0.0 | 0.0 | 0.0 | 0.0 |
| neuro*; met SK-N-AS | 0.0 | 0.0 | 0.0 | 0.0 | 0.0 |
| astrocytoma SF-539 | 0.0 | 0.0 | 0.0 | 0.0 | 0.0 |
| astrocytoma SNB-75 | 0.0 | 0.0 | 0.0 | 0.0 | 0.0 |
| glioma SNB-19 | 0.0 | 0.0 | 0.0 | 0.0 | 0.0 |
| glioma U251 | 0.0 | 0.0 | 0.0 | 0.0 | 0.0 |
| glioma SF-295 | 0.0 | 0.0 | 0.0 | 0.0 | 0.0 |
| Heart (fetal) | 1.8 | 0.7 | 2.1 | 2.1 | 1.5 |
| Heart | 0.1 | 0.2 | 0.0 | 0.1 | 0.2 |
| Skeletal muscle (fetal) | 8.5 | 6.9 | 9.3 | 9.5 | 6.8 |
| Skeletal muscle | 0.0 | 0.0 | 0.0 | 0.1 | 0.0 |
| Bone marrow | 2.6 | 2.2 | 3.3 | 2.4 | 3.1 |
| Thymus | 0.1 | 0.0 | 0.1 | 0.1 | 0.2 |
| Spleen | 100.0 | 100.0 | 100.0 | 100.0 | 100.0 |
| Lymph node | 20.7 | 17.9 | 25.5 | 26.4 | 32.8 |
| Colorectal | 1.6 | 1.1 | 0.6 | 0.7 | 0.4 |
| Stomach | 1.3 | 0.5 | 1.2 | 0.7 | 0.9 |
| Small intestine | 1.3 | 1.0 | 1.1 | 1.1 | 1.2 |
| Colon ca. SW480 | 0.0 | 0.0 | 0.0 | 0.0 | 0.0 |
| Colon ca.* SW620(SW480 met) | 0.0 | 0.0 | 0.0 | 0.0 | 0.0 |
| Colon ca. HT29 | 0.0 | 0.0 | 0.0 | 0.0 | 0.0 |
| Colon ca. HCT-116 | 0.0 | 0.0 | 0.0 | 0.0 | 0.0 |
| Colon ca. CaCo-2 | 0.0 | 0.0 | 0.0 | 0.0 | 0.0 |
| Colon ca. tissue(ODO3866) | 0.1 | 0.1 | 0.0 | 0.2 | 0.1 |
| Colon ca. HCC-2998 | 0.0 | 0.0 | 0.0 | 0.0 | 0.0 |
| Gastric ca.* (liver met) NCI-N87 | 0.0 | 0.0 | 0.0 | 0.0 | 0.1 |
| Bladder | 0.3 | 0.4 | 0.6 | 0.6 | 0.4 |
| Trachea | 1.3 | 0.7 | 1.4 | 1.2 | 1.3 |
| Kidney | 0.0 | 0.0 | 0.1 | 0.0 | 0.0 |
| Kidney (fetal) | 2.4 | 3.4 | 3.8 | 2.1 | 3.0 |
| Renal ca. 786-0 | 0.0 | 0.0 | 0.0 | 0.0 | 0.0 |
| Renal ca. A498 | 0.0 | 0.0 | 0.0 | 0.0 | 0.0 |
| Renal ca. RXF 393 | 0.0 | 0.0 | 0.0 | 0.0 | 0.0 |
| Renal ca. ACHN | 0.0 | 0.0 | 0.0 | 0.0 | 0.0 |
| Renal ca. UO-31 | 0.0 | 0.0 | 0.0 | 0.0 | 0.0 |
| Renal ca. TK-10 | 0.0 | 0.0 | 0.0 | 0.0 | 0.0 |
| Liver | 5.6 | 8.8 | 6.1 | 6.4 | 10.3 |
| Liver (fetal) | 33.4 | 33.2 | 33.9 | 33.4 | 36.6 |
| Liver ca. (hepatoblast) HepG2 | 0.0 | 0.0 | 0.0 | 0.0 | 0.0 |
| Lung | 0.6 | 0.6 | 0.6 | 0.7 | 0.2 |
| Lung (fetal) | 2.0 | 1.9 | 2.4 | 1.0 | 3.1 |
| Lung ca. (small cell) LX-1 | 0.0 | 0.0 | 0.0 | 0.0 | 0.0 |
| Lung ca. (small cell) NCI-H69 | 0.0 | 0.0 | 0.0 | 0.0 | 0.0 |
| Lung ca. (s. cell var.) SHP-77 | 0.0 | 0.0 | 0.0 | 0.0 | 0.0 |
| Lung ca. (large cell)NCI-H460 | 0.0 | 0.0 | 0.0 | 0.0 | 0.0 |
| Lung ca. (non-sm. cell) A549 | 0.0 | 0.0 | 0.0 | 0.0 | 0.0 |
| Lung ca. (non-s. cell) NCI-H23 | 0.0 | 0.0 | 0.0 | 0.0 | 0.0 |
| Lung ca. (non-s. cell) HOP-62 | 0.0 | 0.0 | 0.0 | 0.0 | 0.0 |
| Lung ca. (non-s. cl) NCI-H522 | 0.0 | 0.0 | 0.0 | 0.0 | 0.0 |
| Lung ca. (squam.) SW 900 | 0.0 | 0.0 | 0.0 | 0.0 | 0.0 |
| Lung ca. (squam.) NCI-H596 | 0.0 | 0.0 | 0.0 | 0.0 | 0.0 |
| Mammary gland | 1.6 | 1.5 | 1.1 | 1.7 | 1.5 |
| Breast ca.* (pl. ef) MCF-7 | 0.0 | 0.0 | 0.0 | 0.0 | 0.0 |
| Breast ca.* (pl. ef) MDA-MB-231 | 0.0 | 0.0 | 0.0 | 0.0 | 0.0 |
| Breast ca.* (pl. ef) T47D | 0.0 | 0.0 | 0.0 | 0.0 | 0.0 |
| Breast ca. BT-549 | 0.0 | 0.0 | 0.0 | 0.0 | 0.0 |
| Breast ca. MDA-N | 0.0 | 0.0 | 0.0 | 0.0 | 0.0 |
| Ovary | 5.0 | 5.4 | 4.5 | 6.0 | 4.6 |
| Ovarian ca. OVCAR-3 | 0.0 | 0.0 | 0.0 | 0.0 | 0.0 |
| Ovarian ca. OVCAR-4 | 0.0 | 0.0 | 0.0 | 0.0 | 0.0 |
| Ovarian ca. OVCAR-5 | 0.0 | 0.0 | 0.0 | 0.0 | 0.0 |
| Ovarian ca. OVCAR-8 | 0.0 | 0.0 | 0.0 | 0.0 | 0.0 |
| Ovarian ca. IGROV-1 | 0.0 | 0.0 | 0.0 | 0.0 | 0.0 |
| Ovarian ca.* (ascites) SK-OV-3 | 0.0 | 0.0 | 0.0 | 0.0 | 0.0 |
| Uterus | 0.2 | 0.2 | 0.5 | 0.3 | 0.1 |
| Placenta | 0.4 | 0.0 | 0.4 | 0.1 | 0.2 |
| Prostate | 0.2 | 0.1 | 0.1 | 0.1 | 0.1 |
| Prostate ca.* (bone met)PC-3 | 0.0 | 0.0 | 0.0 | 0.0 | 0.0 |
| Testis | 7.5 | 5.9 | 4.7 | 6.5 | 5.6 |
| Melanoma Hs688(A).T | 0.0 | 0.0 | 0.0 | 0.0 | 0.0 |
| Melanoma* (met) Hs688(B).T | 0.0 | 0.0 | 0.0 | 0.0 | 0.0 |
| Melanoma UACC-62 | 0.0 | 0.0 | 0.0 | 0.0 | 0.0 |
| Melanoma M14 | 0.0 | 0.0 | 0.0 | 0.0 | 0.0 |
| Melanoma LOX IMVI | 0.0 | 0.0 | 0.0 | 0.0 | 0.0 |
| Melanoma* (met) SK-MEL-5 | 0.0 | 0.0 | 0.0 | 0.0 | 0.0 |
| Adipose | 1.0 | 1.5 | 0.6 | 0.5 | 1.1 |

Column A - Rel. Exp. (%) Ag2742, Run 153641674
Column B - Rel. Exp. (%) Ag2743, Run 153658349
Column C - Rel. Exp. (%) Ag2744, Run 153670718
Column D - Rel. Exp. (%) Ag2745, Run 153664738
Column E - Rel. Exp. (%) Ag2746, Run 153675151

TABLE HM
Panel 2D

| Tissue Name | A | B | C | D | E |
|---|---|---|---|---|---|
| Normal Colon | 3.2 | 3.8 | 4.4 | 4.8 | 4.5 |
| CC Well to Mod Diff (ODO3866) | 0.1 | 0.1 | 0.1 | 0.0 | 0.0 |
| CC Margin (ODO3866) | 0.7 | 0.5 | 0.9 | 1.2 | 0.3 |
| CC Gr.2 rectosigmoid (ODO3868) | 0.2 | 0.4 | 0.2 | 0.2 | 0.1 |
| CC Margin (ODO3868) | 0.1 | 0.1 | 0.0 | 0.2 | 0.1 |
| CC Mod Diff (ODO3920) | 0.0 | 0.1 | 0.1 | 0.0 | 0.0 |
| CC Margin (ODO3920) | 1.1 | 2.0 | 3.1 | 1.0 | 1.4 |
| CC Gr.2 ascend colon (ODO3921) | 0.2 | 0.3 | 0.9 | 0.5 | 0.7 |
| CC Margin (ODO3921) | 0.7 | 0.3 | 0.9 | 1.0 | 0.2 |
| CC from Partial Hepatectomy (ODO4309) Mets | 7.6 | 8.4 | 10.2 | 9.5 | 8.8 |
| Liver Margin (ODO4309) | 100.0 | 100.0 | 100.0 | 100.0 | 100.0 |
| Colon mets to lung (OD04451-01) | 0.4 | 0.3 | 0.3 | 1.5 | 0.4 |
| Lung Margin (OD04451-02) | 0.2 | 0.1 | 0.8 | 0.2 | 0.2 |
| Normal Prostate 6546-1 | 0.4 | 0.0 | 0.2 | 0.1 | 0.1 |
| Prostate Cancer (OD04410) | 0.2 | 0.2 | 0.0 | 0.3 | 0.0 |
| Prostate Margin (OD04410) | 0.0 | 0.2 | 0.3 | 0.0 | 0.6 |
| Prostate Cancer (OD04720-01) | 0.6 | 0.1 | 0.2 | 0.2 | 0.3 |
| Prostate Margin (OD04720-02) | 0.5 | 0.5 | 0.6 | 0.3 | 0.2 |
| Normal Lung 061010 | 5.6 | 5.2 | 6.3 | 8.1 | 5.9 |
| Lung Met to Muscle (ODO4286) | 0.0 | 0.0 | 0.0 | 0.0 | 0.0 |
| Muscle Margin (ODO4286) | 0.5 | 0.0 | 0.1 | 0.2 | 0.0 |
| Lung Malignant Cancer (OD03126) | 1.0 | 1.0 | 1.3 | 0.8 | 1.0 |
| Lung Margin (OD03126) | 0.9 | 1.1 | 0.9 | 1.4 | 1.4 |
| Lung Cancer (OD04404) | 1.3 | 0.8 | 1.6 | 1.5 | 1.4 |
| Lung Margin (OD04404) | 2.0 | 2.5 | 4.3 | 3.4 | 3.2 |
| Lung Cancer (OD04565) | 0.2 | 0.1 | 0.5 | 0.0 | 0.2 |
| Lung Margin (OD04565) | 0.3 | 0.0 | 0.3 | 0.4 | 0.7 |
| Lung Cancer (OD04237-01) | 0.4 | 0.9 | 1.2 | 1.5 | 1.2 |
| Lung Margin (OD04237-02) | 5.6 | 5.4 | 7.9 | 6.3 | 5.6 |
| Ocular Mel Met to Liver (ODO4310) | 0.2 | 0.0 | 0.0 | 0.1 | 0.4 |
| Liver Margin (ODO4310) | 52.9 | 64.6 | 79.6 | 81.8 | 63.3 |
| Melanoma Mets to Lung (OD04321) | 0.0 | 0.0 | 0.0 | 0.1 | 0.3 |
| Lung Margin (OD04321) | 0.2 | 2.0 | 0.9 | 1.5 | 0.5 |
| Normal Kidney | 0.5 | 0.3 | 0.3 | 0.7 | 0.3 |
| Kidney Ca, Nuclear grade 2 (OD04338) | 0.0 | 0.1 | 0.0 | 0.0 | 0.1 |
| Kidney Margin (OD04338) | 0.0 | 0.2 | 0.5 | 0.1 | 0.3 |
| Kidney Ca Nuclear grade 1/2 (OD04339) | 0.0 | 0.0 | 0.0 | 0.0 | 0.0 |
| Kidney Margin (OD04339) | 0.0 | 0.1 | 0.1 | 0.3 | 0.1 |
| Kidney Ca, Clear cell type (OD04340) | 0.0 | 0.1 | 0.0 | 0.0 | 0.1 |
| Kidney Margin (OD04340) | 0.2 | 0.1 | 0.8 | 0.0 | 0.0 |
| Kidney Ca, Nuclear grade 3 (OD04348) | 0.0 | 0.0 | 0.2 | 0.0 | 0.1 |
| Kidney Margin (OD04348) | 0.0 | 0.1 | 0.2 | 0.1 | 0.1 |
| Kidney Cancer (OD04622-01) | 0.2 | 0.1 | 0.1 | 1.2 | 0.2 |
| Kidney Margin (OD04622-03) | 0.0 | 0.1 | 0.0 | 0.0 | 0.0 |
| Kidney Cancer (OD04450-01) | 0.0 | 0.0 | 0.0 | 0.0 | 0.0 |
| Kidney Margin (OD04450-03) | 0.1 | 0.0 | 0.1 | 0.1 | 0.1 |
| Kidney Cancer 8120607 | 0.0 | 0.0 | 0.0 | 0.0 | 0.0 |
| Kidney Margin 8120608 | 0.1 | 0.0 | 0.2 | 0.0 | 0.0 |
| Kidney Cancer 8120613 | 0.3 | 0.2 | 0.2 | 0.1 | 0.5 |
| Kidney Margin 8120614 | 0.3 | 0.1 | 0.4 | 0.3 | 0.0 |
| Kidney Cancer 9010320 | 0.0 | 0.1 | 0.1 | 0.1 | 0.2 |
| Kidney Margin 9010321 | 0.1 | 0.0 | 0.0 | 0.1 | 0.2 |
| Normal Uterus | 0.5 | 0.1 | 0.8 | 0.1 | 0.5 |
| Uterus Cancer 064011 | 0.8 | 0.7 | 1.3 | 0.4 | 0.6 |
| Normal Thyroid | 1.8 | 1.1 | 1.5 | 0.9 | 2.3 |
| Thyroid Cancer 064010 | 0.0 | 0.0 | 0.0 | 0.0 | 0.0 |
| Thyroid Cancer A302152 | 0.5 | 1.0 | 1.3 | 0.6 | 0.8 |
| Thyroid Margin A302153 | 2.3 | 2.4 | 3.5 | 3.9 | 2.4 |
| Normal Breast | 9.0 | 6.8 | 9.0 | 9.9 | 5.5 |
| Breast Cancer (OD04566) | 0.1 | 0.0 | 0.1 | 0.0 | 0.0 |
| Breast Cancer (OD04590-01) | 0.3 | 0.4 | 0.2 | 0.7 | 0.2 |
| Breast Cancer Mets (OD04590-03) | 1.7 | 2.4 | 2.5 | 2.5 | 1.4 |
| Breast Cancer Metastasis (OD04655-05) | 10.7 | 13.2 | 22.1 | 15.6 | 12.8 |
| Breast Cancer 064006 | 0.5 | 0.8 | 0.8 | 0.7 | 0.7 |
| Breast Cancer 1024 | 4.1 | 2.5 | 5.1 | 3.1 | 3.6 |
| Breast Cancer 9100266 | 0.9 | 0.2 | 0.2 | 0.2 | 0.2 |
| Breast Margin 9100265 | 0.7 | 0.4 | 1.0 | 0.7 | 1.2 |
| Breast Cancer A209073 | 1.2 | 1.2 | 1.1 | 1.9 | 0.8 |
| Breast Margin A209073 | 1.5 | 1.0 | 2.1 | 1.5 | 0.6 |
| Normal Liver | 27.5 | 27.9 | 37.4 | 42.3 | 27.2 |
| Liver Cancer 064003 | 0.8 | 0.5 | 0.3 | 0.6 | 0.5 |
| Liver Cancer 1025 | 33.7 | 36.6 | 36.6 | 39.0 | 27.5 |
| Liver Cancer 1026 | 4.6 | 3.6 | 5.7 | 5.8 | 5.2 |
| Liver Cancer 6004-T | 36.9 | 38.7 | 50.3 | 46.7 | 38.7 |
| Liver Tissue 6004-N | 1.5 | 1.0 | 1.3 | 1.3 | 1.6 |
| Liver Cancer 6005-T | 4.5 | 3.9 | 4.2 | 4.3 | 2.9 |
| Liver Tissue 6005-N | 22.2 | 24.5 | 32.8 | 27.5 | 28.7 |
| Normal Bladder | 5.3 | 3.4 | 4.8 | 4.2 | 5.5 |
| Bladder Cancer 1023 | 1.6 | 1.3 | 1.5 | 1.6 | 1.7 |
| Bladder Cancer A302173 | 0.9 | 0.4 | 1.5 | 1.0 | 0.3 |
| Bladder Cancer (OD04718-01) | 0.0 | 0.0 | 0.0 | 0.0 | 0.0 |
| Bladder Normal Adjacent (OD04718-03) | 5.2 | 6.2 | 7.8 | 6.0 | 4.1 |
| Normal Ovary | 1.6 | 3.0 | 5.4 | 3.6 | 3.6 |
| Ovarian Cancer 064008 | 0.0 | 0.1 | 0.1 | 0.6 | 0.5 |
| Ovarian Cancer (OD04768-07) | 0.0 | 0.0 | 0.0 | 0.1 | 0.0 |
| Ovary Margin (OD04768-08) | 2.3 | 1.7 | 3.1 | 2.4 | 2.6 |
| Normal Stomach | 0.9 | 0.8 | 1.7 | 0.8 | 0.8 |
| Gastric Cancer 9060358 | 0.0 | 0.4 | 0.8 | 0.4 | 0.2 |
| Stomach Margin 9060359 | 1.2 | 0.8 | 1.3 | 1.0 | 0.9 |
| Gastric Cancer 9060395 | 0.1 | 0.2 | 0.3 | 0.5 | 0.6 |
| Stomach Margin 9060394 | 0.8 | 0.6 | 1.7 | 0.7 | 0.6 |
| Gastric Cancer 9060397 | 0.1 | 0.1 | 0.1 | 0.0 | 0.0 |
| Stomach Margin 9060396 | 0.3 | 0.1 | 0.6 | 0.1 | 0.3 |
| Gastric Cancer 064005 | 0.7 | 0.6 | 0.4 | 1.1 | 0.3 |

Column A - Rel. Exp. (%) Ag2742, Run 153641758
Column B - Rel. Exp. (%) Ag2743, Run 153658357
Column C - Rel. Exp. (%) Ag2744, Run 153670751
Column D - Rel. Exp. (%) Ag2745, Run 153664739
Column E - Rel. Exp. (%) Ag2746, Run 153675220

TABLE HN
Panel 4D

| Tissue Name | A | B | C | D | E | F |
|---|---|---|---|---|---|---|
| Secondary Th1 act | 0.0 | 0.0 | 0.0 | 0.0 | 0.0 | 0.0 |
| Secondary Th2 act | 0.0 | 0.7 | 0.0 | 0.0 | 0.0 | 0.0 |
| Secondary Tr1 act | 1.7 | 0.0 | 1.9 | 0.0 | 7.5 | 2.8 |
| Secondary Th1 rest | 0.0 | 0.0 | 0.0 | 0.0 | 0.0 | 0.0 |
| Secondary Th2 rest | 0.0 | 0.0 | 0.0 | 0.0 | 0.0 | 0.0 |
| Secondary Tr1 rest | 0.0 | 0.0 | 0.0 | 0.0 | 0.0 | 2.8 |
| Primary Th1 act | 0.0 | 0.0 | 0.0 | 0.0 | 0.0 | 0.0 |
| Primary Th2 act | 1.6 | 0.0 | 0.0 | 0.0 | 0.0 | 4.8 |
| Primary Tr1 act | 0.0 | 0.0 | 0.0 | 0.0 | 0.0 | 0.0 |
| Primary Th1 rest | 0.0 | 0.0 | 0.0 | 0.0 | 0.0 | 0.0 |
| Primary Th2 rest | 0.0 | 0.0 | 0.0 | 0.0 | 0.0 | 0.0 |
| Primary Tr1 rest | 0.0 | 0.0 | 0.0 | 0.0 | 0.0 | 0.0 |
| CD45RA CD4 lymphocyte act | 0.0 | 0.0 | 0.0 | 0.0 | 0.0 | 0.0 |
| CD45RO CD4 lymphocyte act | 0.0 | 0.0 | 0.0 | 0.0 | 0.0 | 0.0 |
| CD8 lymphocyte act | 0.0 | 0.0 | 0.0 | 0.0 | 0.0 | 0.0 |
| Secondary CD8 lymphocyte rest | 0.0 | 0.0 | 0.0 | 0.0 | 0.0 | 0.0 |
| Secondary CD8 lymphocyte act | 0.7 | 0.0 | 0.0 | 7.8 | 0.0 | 1.8 |
| CD4 lymphocyte none | 0.0 | 0.0 | 0.0 | 0.0 | 0.0 | 0.0 |
| 2ry Th1/Th2/Tr1_anti-CD95 CH11 | 0.0 | 0.0 | 0.0 | 0.0 | 0.0 | 0.0 |
| LAK cells rest | 0.0 | 0.0 | 0.0 | 0.0 | 0.0 | 0.0 |
| LAK cells IL-2 | 0.0 | 0.0 | 0.0 | 0.0 | 0.0 | 4.7 |
| LAK cells IL-2 + IL-12 | 0.0 | 0.0 | 0.0 | 0.0 | 1.9 | 0.0 |
| LAK cells IL-2 + IFN gamma | 0.0 | 1.5 | 3.3 | 5.0 | 0.0 | 3.0 |
| LAK cells IL-2 + IL-18 | 0.0 | 0.0 | 2.1 | 0.0 | 0.0 | 0.0 |
| LAK cells PMA/ionomycin | 0.0 | 0.0 | 0.0 | 0.0 | 0.0 | 0.0 |
| NK Cells IL-2 rest | 0.0 | 0.0 | 0.0 | 0.0 | 0.0 | 0.0 |
| Two Way MLR 3 day | 0.0 | 0.0 | 0.0 | 0.0 | 0.0 | 0.0 |
| Two Way MLR 5 day | 0.0 | 0.0 | 0.0 | 0.0 | 0.0 | 0.0 |
| Two Way MLR 7 day | 2.0 | 0.0 | 6.4 | 0.0 | 0.0 | 11.3 |
| PBMC rest | 0.0 | 0.0 | 0.0 | 0.0 | 0.0 | 5.1 |
| PBMC PWM | 0.0 | 2.3 | 0.0 | 0.0 | 0.0 | 2.7 |
| PBMC PHA-L | 0.0 | 1.5 | 2.5 | 0.0 | 0.0 | 2.2 |
| Ramos (B cell) none | 0.0 | 0.0 | 0.0 | 0.0 | 0.0 | 0.0 |
| Ramos (B cell) ionomycin | 0.0 | 0.0 | 0.0 | 0.0 | 0.0 | 0.0 |
| B lymphocytes PWM | 0.0 | 0.0 | 0.0 | 0.0 | 0.0 | 2.0 |
| B lymphocytes CD40L and IL-4 | 0.0 | 0.0 | 0.0 | 0.0 | 0.0 | 6.0 |
| EOL-1 dbcAMP | 0.0 | 0.0 | 0.0 | 0.0 | 0.0 | 0.0 |
| EOL-1 dbcAMP PMA/ionomycin | 0.0 | 0.0 | 0.0 | 0.0 | 0.0 | 0.0 |
| Dendritic cells none | 1.4 | 2.9 | 0.0 | 0.0 | 4.9 | 0.0 |
| Dendritic cells LPS | 0.0 | 0.0 | 0.0 | 0.0 | 0.0 | 0.0 |
| Dendritic cells anti-CD40 | 0.0 | 0.0 | 3.3 | 0.0 | 0.0 | 0.0 |
| Monocytes rest | 0.0 | 0.0 | 0.0 | 0.0 | 0.0 | 0.0 |
| Monocytes LPS | 0.0 | 0.0 | 0.0 | 0.0 | 0.0 | 0.0 |
| Macrophages rest | 0.0 | 0.0 | 0.0 | 0.0 | 0.0 | 0.0 |
| Macrophages LPS | 0.0 | 0.0 | 0.0 | 2.4 | 0.0 | 0.0 |
| HUVEC none | 0.0 | 0.0 | 0.0 | 0.0 | 0.0 | 0.0 |
| HUVEC starved | 0.0 | 0.0 | 0.0 | 0.0 | 0.0 | 0.0 |
| HUVEC IL-1beta | 0.0 | 0.0 | 0.0 | 0.0 | 0.0 | 0.0 |
| HUVEC IFN gamma | 3.7 | 0.0 | 0.0 | 0.0 | 2.7 | 0.0 |
| HUVEC TNF alpha + IFN gamma | 0.0 | 0.0 | 0.0 | 0.0 | 0.0 | 0.0 |
| HUVEC TNF alpha + IL4 | 0.0 | 0.0 | 0.0 | 0.0 | 0.0 | 0.0 |
| HUVEC IL-11 | 1.4 | 0.0 | 0.0 | 0.0 | 0.0 | 2.6 |
| Lung Microvascular EC none | 1.6 | 0.0 | 2.2 | 0.0 | 0.0 | 0.0 |
| Lung Microvascular EC TNFalpha + IL-1beta | 3.1 | 2.9 | 0.0 | 0.0 | 0.0 | 0.0 |
| Microvascular Dermal EC none | 0.0 | 3.1 | 5.0 | 0.0 | 0.0 | 12.6 |
| Microsvasular Dermal EC TNFalpha + IL-1beta | 1.4 | 1.1 | 0.0 | 0.0 | 1.6 | 0.0 |
| Bronchial epithelium TNFalpha + IL1beta | 0.0 | 0.0 | 0.0 | 0.0 | 0.0 | 0.0 |
| Small airway epithelium none | 0.0 | 0.0 | 0.0 | 0.0 | 0.0 | 0.0 |
| Small airway epithelium TNFalpha + IL-1beta | 0.0 | 0.0 | 0.0 | 0.0 | 0.0 | 0.0 |
| Coronery artery SMC rest | 0.0 | 0.0 | 0.0 | 0.0 | 0.0 | 0.0 |
| Coronery artery SMC TNFalpha + IL-1beta | 0.0 | 0.0 | 0.0 | 0.0 | 0.0 | 0.0 |
| Astrocytes rest | 0.0 | 0.0 | 0.0 | 0.0 | 0.0 | 0.0 |
| Astrocytes TNFalpha + IL-1 beta | 0.0 | 0.0 | 0.0 | 0.0 | 0.0 | 0.0 |
| KU-812 (Basophil) rest | 0.0 | 0.0 | 0.0 | 0.0 | 0.0 | 0.0 |
| KU-812 (Basophil) PMA/ionomycin | 0.0 | 0.0 | 0.0 | 0.0 | 0.0 | 0.0 |
| CCD1106 (Keratinocytes) none | 0.0 | 0.0 | 0.0 | 0.0 | 0.0 | 0.0 |
| CCD1106 (Keratinocytes) TNFalpha + IL-1beta | 0.0 | 0.0 | 0.0 | 0.0 | 0.0 | 0.0 |
| Liver cirrhosis | 100.0 | 100.0 | 100.0 | 100.0 | 100.0 | 100.0 |
| Lupus kidney | 0.0 | 1.4 | 0.0 | 0.0 | 0.0 | 0.0 |
| NCI-H292 none | 0.0 | 0.0 | 0.0 | 0.0 | 0.0 | 0.0 |
| NCI-H292 IL-4 | 0.0 | 0.0 | 0.0 | 0.0 | 2.3 | 0.0 |
| NCI-H292 IL-9 | 0.0 | 0.0 | 0.0 | 0.0 | 0.0 | 0.0 |
| NCI-H292 IL-13 | 0.0 | 0.0 | 0.0 | 0.0 | 0.0 | 0.0 |
| NCI-H292 IFN gamma | 0.0 | 0.0 | 0.0 | 0.0 | 0.0 | 0.0 |
| HPAEC none | 4.8 | 1.4 | 0.0 | 0.0 | 0.0 | 3.0 |
| HPAEC TNF alpha + IL-1 beta | 0.0 | 0.0 | 0.0 | 0.0 | 0.0 | 0.0 |
| Lung fibroblast none | 0.0 | 0.0 | 0.0 | 0.0 | 0.0 | 0.0 |
| Lung fibroblast TNF alpha + IL-1 beta | 0.0 | 0.0 | 0.0 | 0.0 | 0.0 | 0.0 |
| Lung fibroblast IL-4 | 0.0 | 0.0 | 0.0 | 0.0 | 0.0 | 0.0 |
| Lung fibroblast IL-9 | 0.0 | 0.0 | 0.0 | 0.0 | 0.0 | 0.0 |
| Lung fibroblast IL-13 | 0.0 | 0.0 | 0.0 | 0.0 | 0.0 | 0.0 |
| Lung fibroblast IFN gamma | 0.0 | 0.0 | 0.0 | 0.0 | 0.0 | 0.0 |
| Dermal fibroblast CCD1070 rest | 0.0 | 0.0 | 0.0 | 0.0 | 0.0 | 0.0 |
| Dermal fibroblast CCD1070 TNF alpha | 0.0 | 0.0 | 0.0 | 0.0 | 0.0 | 0.0 |
| Dermal fibroblast CCD1070 IL-1 beta | 0.0 | 0.0 | 0.0 | 0.0 | 0.0 | 0.0 |
| Dermal fibroblast IFN gamma | 0.0 | 0.0 | 0.0 | 0.0 | 0.0 | 5.3 |
| Dermal fibroblast IL-4 | 0.0 | 0.0 | 0.0 | 0.0 | 0.0 | 0.0 |
| IBD Colitis 2 | 1.7 | 0.0 | 0.0 | 0.0 | 0.0 | 0.0 |
| IBD Crohn's | 4.5 | 1.6 | 6.7 | 6.1 | 3.3 | 3.0 |
| Colon | 5.5 | 2.1 | 23.0 | 8.3 | 4.7 | 11.1 |
| Lung | 41.5 | 26.2 | 27.7 | 39.2 | 37.1 | 49.7 |
| Thymus | 4.2 | 0.0 | 4.1 | 0.0 | 1.4 | 20.6 |
| Kidney | 4.7 | 5.2 | 8.7 | 5.9 | 6.3 | 6.7 |

Column A - Rel. Exp. (%) Ag2742, Run 153641803
Column B - Rel. Exp. (%) Ag2743, Run 153658360
Column C - Rel. Exp. (%) Ag2744, Run 153670759
Column D - Rel. Exp. (%) Ag2745, Run 153664740
Column E - Rel. Exp. (%) Ag2746, Run 153675321
Column F - Rel. Exp. (%) Ag812, Run 138175358

TABLE HO
general oncology screening panel_v_2.4

| Tissue Name | A |
|---|---|
| Colon cancer 1 | 2.9 |
| Colon NAT 1 | 3.9 |
| Colon cancer 2 | 10.2 |
| Colon NAT 2 | 5.0 |
| Colon cancer 3 | 4.8 |
| Colon NAT 3 | 10.2 |
| Colon malignant cancer 4 | 0.0 |
| Colon NAT 4 | 3.8 |
| Lung cancer 1 | 5.0 |
| Lung NAT 1 | 1.3 |
| Lung cancer 2 | 9.0 |
| Lung NAT 2 | 0.0 |
| Squamous cell carcinoma 3 | 6.1 |
| Lung NAT 3 | 0.0 |
| Metastatic melanoma 1 | 43.8 |
| Melanoma 2 | 0.5 |
| Melanoma 3 | 4.0 |
| Metastatic melanoma 4 | 82.4 |
| Metastatic melanoma 5 | 100.0 |
| Bladder cancer 1 | 1.8 |
| Bladder NAT 1 | 0.0 |
| Bladder cancer 2 | 0.7 |
| Bladder NAT 2 | 0.7 |
| Bladder NAT 3 | 0.0 |
| Bladder NAT 4 | 9.0 |
| Prostate adenocarcinoma 1 | 0.7 |
| Prostate adenocarcinoma 2 | 0.6 |
| Prostate adenocarcinoma 3 | 0.9 |
| Prostate adenocarcinoma 4 | 0.9 |
| Prostate NAT 5 | 2.3 |
| Prostate adenocarcinoma 6 | 0.0 |
| Prostate adenocarcinoma 7 | 0.0 |
| Prostate adenocarcinoma 8 | 1.0 |
| Prostate adenocarcinoma 9 | 0.7 |
| Prostate NAT 10 | 0.2 |
| Kidney cancer 1 | 1.1 |
| Kidney NAT 1 | 1.6 |
| Kidney cancer 2 | 16.2 |
| Kidney NAT 2 | 3.0 |
| Kidney cancer 3 | 4.7 |
| Kidney NAT 3 | 0.0 |
| Kidney cancer 4 | 0.0 |
| Kidney NAT 4 | 0.0 |

Column A - Rel. Exp. (%) Ag812, Run 262228032

Panel 1 Summary: Ag03/Ag068 Highest expression of this gene was detected in the spleen (CTs=21-25). This gene was more highly expressed in normal tissue than in cancer cell lines. There were however detectable levels of expression in cell lines derived from melanoma, breast, renal, ovarian, lung, gastric and colon cancers. The difference in levels of expression of this gene is useful for differentiating between these cancer cell line samples and other samples on this panel and between normal tissues and malignancies from those cancers.

There were also higher levels of expression in lung, and kidney tissue from fetal sources (CTs=25-28) when compared to levels of expression in the adult (CTs=38-31). 10 Expression of this gene or its protein product is useful for differentiating between adult and fetal lung and kidney tissue.

Among tissues with metabolic function, this gene was expressed in the liver, pituitary, thyroid, heart, skeletal muscle and adrenal gland. This demonstrates that the protein encoded by this gene is involved in the homeostasis of these tissues. Therapeutic 15 modulation of the expression or function of this gene product is effective in the treatment of metabolic disorders, including obesity and diabetes.

This gene is a homolog of Stablin-1, and was also expressed at moderate levels in all brain regions examined. Because stablin is involved in angiogenesis, the therapeutic modulation of this gene or its protein product is of benefit in the treatment of 20 stroke/cerebral ischemia/cerebral infarct.

Panel 1.2 Summary: Ag812 Highest expression of this gene was detected in the liver (CTs=25). Significant expression was also found in other metabolic tissues including fetal and adult heart, skeletal muscle, pancreas, thyroid, pituitary and adrenal gland. The high expression of this gene in the liver shows that this gene is involved in the normal homeostasis of that organ. Therapeutic modulation of the expression or function of this gene or protein product is effective in the treatment of diseases that involve the liver.

This gene also showed low to moderate expression in the brain. Please see Panel 1 for discussion of potential utility of this gene in the central nervous system.

While this gene showed a greater association for normal tissue, there were significant levels of expression in a cluster of ovarian cancer cell lines. Expression of this gene is useful for differentiating between those samples and other samples on this panel, and between normal and malignant ovarian tissue. Therapeutic modulation of the expression or function of this gene or protein product is effective in the treatment of ovarian cancer.

Panel 1.3D Summary: Ag2742, Ag2743, Ag2744, Ag2745, Ag2746 Highest expression of this gene was detected in the liver (CTs=25). Significant expression was also found in the spleen (CTs=28-29). This result was in concordance with the results from Panel 1.

This gene was expressed at higher levels in the fetal kidney and skeletal muscle (CTs=32-34) than in the comparable adult tissues (CTs=40). Thus, expression of this gene is useful for differentiating between kidney and skeletal muscle tissue from adult and fetal sources. The higher levels of expression of this gene in the fetal tissues show that this gene product is involved in the development of these organs. Therapeutic modulation of the expression or function of these genes or their protein products is effective in treating disease of these organs in the adult.

In this panel, expression this gene was exclusively associate with normal tissue samples, a preference that was also observed in panels 1 and 1.2. Absence of expression of this gene is useful in differentiating between the cancerous cell lines on this panel, and their corresponding normal tissues.

Panel 2D Summary: Ag2742/Ag2743/Ag2744/Ag2745/Ag2746 Expression of this gene was highest and almost exclusive in the liver (CTs=27-29). This result was in concordance with the results from previous panels. The low/undetectable levels of expression in cancer samples was also in agreement with the results observed in the preceding experiments. Expression of this gene or its protein product is useful for differentiating between liver tissue and other samples on this panel and as a marker for liver tissue. Therapeutic modulation of the expression or function of the gene or the protein encoded by this gene is effective in the treatment of liver cancer or other disease that involve the liver. This gene is useful as a marker for breast cancer metastasis as it shows elevated expression in a metastatic breast cancer compared to expression in the primary cancer.

Panel 4D Summary: Ag812/Ag2742/Ag2743/Ag2744/Ag2745/Ag2746 The expression of this gene appears was highest in samples from cirrhotic liver, (CTs=32-33). Low level expression was also detected in samples derived from normal lung. The presence of this gene in liver cirrhosis (a component of which involves liver inflammation and fibrosis) shows that therapeutic agents involving this gene or its protein product are useful in reducing or inhibiting the inflammation associated with fibrotic and other inflammatory diseases.

general oncology screening panel_V_{—}2.4 Summary: Ag812 Highest expression of this gene was seen in metastatic melanoma (CTs=31). Gene, protein, antibodies or small molecule therapeutics that target this gene or its protein product are useful in the treatment of melanoma.

I. CG50925-01 and CG50925-02: TENM5

Expression of gene CG50925-01 and variant CG50925-02 was assessed using the primer-probe set Ag1402 described in Table IA. Results of the RTQ-PCR runs are shown in Tables B, IC, ID, IE, IF, IG, and IH.

TABLE IA
Probe Name Ag1402

| | | | | |
|---|---|---|---|---|
| | | | Start | SEQ ID |
| Primers | Sequences | Length | Position | No |

| | | | | |
|---|---|---|---|---|
| Forward | 5′-cttacaacaccacgggactaag-3′ | 22 | 5546 | 1291 |
| Probe | TET-5′-aagagcacttccttgcctccgtaagg-3′- | 26 | 5569 | 1292 |
| | TAMRA | | | |
| Reverse | 5′-agatcaaatgattgggatggtt-3′ | 22 | 5605 | 1293 |

TABLE IB
HASS Panel v1.0

| Tissue Name | A |
|---|---|
| MCF-7 C1 | 2.4 |
| MCF-7 C2 | 4.2 |
| MCF-7 C3 | 3.6 |
| MCF-7 C4 | 4.9 |
| MCF-7 C5 | 4.2 |
| MCF-7 C6 | 5.2 |
| MCF-7 C7 | 3.3 |
| MCF-7 C9 | 3.4 |
| MCF-7 C10 | 6.7 |
| MCF-7 C11 | 0.9 |
| MCF-7 C12 | 5.2 |
| MCF-7 C13 | 1.6 |
| MCF-7 C15 | 2.6 |
| MCF-7 C16 | 6.3 |
| MCF-7 C17 | 7.5 |
| T24 D1 | 0.5 |
| T24 D2 | 2.0 |
| T24 D3 | 2.0 |
| T24 D4 | 1.8 |
| T24 D5 | 2.5 |
| T24 D6 | 2.4 |
| T24 D7 | 1.1 |
| T24 D9 | 6.6 |
| T24 D10 | 2.2 |
| T24 D11 | 2.2 |
| T24 D12 | 3.1 |
| T24 D13 | 2.0 |
| T24 D15 | 6.2 |
| T24 D16 | 1.1 |
| T24 D17 | 1.7 |
| CAPaN B1 | 2.3 |
| CAPaN B2 | 0.7 |
| CAPaN B3 | 0.3 |
| CAPaN B4 | 0.3 |
| CAPaN B5 | 1.2 |
| CAPaN B6 | 1.1 |
| CAPaN B7 | 0.4 |
| CAPaN B8 | 0.5 |
| CAPaN B9 | 0.8 |
| CAPaN B10 | 0.9 |
| CAPaN B11 | 0.8 |
| CAPaN B12 | 0.6 |
| CAPaN B13 | 0.7 |
| CAPaN B14 | 1.4 |
| CAPaN B15 | 0.4 |
| CAPaN B16 | 0.6 |
| CAPaN B17 | 1.2 |
| U87-MG F1 (B) | 1.0 |
| U87-MG F2 | 2.7 |
| U87-MG F3 | 5.6 |
| U87-MG F4 | 2.9 |
| U87-MG F5 | 6.7 |
| U87-MG F6 | 7.3 |
| U87-MG F7 | 4.5 |
| U87-MG F8 | 11.7 |
| U87-MG F9 | 3.4 |
| U87-MG F10 | 5.0 |
| U87-MG F11 | 12.9 |
| U87-MG F12 | 7.9 |
| U87-MG F13 | 9.7 |
| U87-MG F14 | 24.3 |
| U87-MG F15 | 10.7 |
| U87-MG F16 | 6.8 |
| U87-MG F17 | 11.4 |
| LnCAP A1 | 0.9 |
| LnCAP A2 | 2.0 |
| LnCAP A3 | 2.2 |
| LnCAP A4 | 2.5 |
| LnCAP A5 | 2.0 |
| LnCAP A6 | 1.8 |
| LnCAP A7 | 5.3 |
| LnCAP A8 | 5.3 |
| LnCAP A9 | 3.0 |
| LnCAP A10 | 1.8 |
| LnCAP A11 | 6.2 |
| LnCAP A12 | 0.5 |
| LnCAP A13 | 0.5 |
| LnCAP A14 | 0.8 |
| LnCAP A15 | 0.8 |
| LnCAP A16 | 1.7 |
| LnCAP A17 | 4.6 |
| Primary Astrocytes | 27.0 |
| Primary Renal Proximal Tubule Epithelial cell A2 | 0.8 |
| Primary melanocytes A5 | 100.0 |
| 126443 - 341 medullo | 0.6 |
| 126444 - 487 medullo | 28.1 |
| 126445 - 425 medullo | 1.2 |
| 126446 - 690 medullo | 8.8 |
| 126447 - 54 adult glioma | 28.1 |
| 126448 - 245 adult glioma | 76.3 |
| 126449 - 317 adult glioma | 9.5 |
| 126450 - 212 glioma | 5.9 |
| 126451 - 456 glioma | 27.0 |

Column A - Rel. Exp. (%) Ag1402, Run 318641615

TABLE IC
Human Metabolic

| Tissue Name | A |
|---|---|
| 137857_psoas-AA.M.Diab.-hi BMI-6 | 0.5 |
| 135760_psoas-HI.M.Diab.-hi BMI-21 | 1.4 |
| 134827_psoas-CC.M.Diab.-hi BMI-4 | 2.1 |
| 137860_psoas-AA.M.Diab.-med BMI-8 | 1.1 |
| 137834_psoas-CC.M.Diab.-med BMI-2 | 2.8 |
| 137828_psoas-CC.M.Diab.-med BMI-1 | 2.1 |
| 135763_psoas-HI.M.Diab.-med BMI-23 | 1.2 |
| 142740_psoas-AS.M.Diab.-low BMI-20 | 18.4 |
| 134834_psoas-AA.M.Diab.-low BMI-17 | 1.2 |
| 137850_psoas-AS.M.Norm-hi BMI-34 | 1.8 |
| 135769_psoas-HI.M.Norm-hi BMI-31 | 0.6 |
| 135766_psoas-AA.M.Norm-hi BMI-25 | 1.3 |
| 142746_psoas-AA.M.Norm-med BMI-37 | 38.2 |
| 142745_psoas-HI.M.Norm-med BMI-35 | 15.7 |
| 137855_psoas-AA.M.Norm-med BMI-47 | 0.9 |
| 137844_psoas-CC.M.Norm-med BMI-26 | 0.5 |
| 142742_psoas-CC.M.Norm-low BMI-40 | 29.9 |
| 137873_psoas-AS.M.Norm-low BMI-28 | 0.6 |
| 137853_psoas-HI.M.Norm-low BMI-41 | 0.9 |
| 135775_psoas-CC.M.Norm-low BMI-39 | 0.5 |
| 137858_diaphragm-AA.M.Diab.-hi BMI-6 | 1.6 |
| 135772_diaphragm-AS.M.Diab-hi BMI-9 | 2.1 |
| 135761_diaphragm-HI.M.Diab.-hi BMI-21 | 5.9 |
| 134828_diaphragm-CC.M.Diab.-hi BMI-4 | 1.1 |
| 137835_diaphragm-CC.M.Diab.-med BMI-2 | 1.6 |
| 135764_diaphragm-HI.M.Diab.-med BMI-23 | 1.8 |
| 134835_diaphragm-AA.M.Diab.-low BMI-17 | 1.8 |
| 142738_diaphragm-CC.M.Norm-hi BMI-29 | 2.1 |
| 139517_diaphragm-AS.M.Norm-hi BMI-34 | 3.3 |
| 137848_diaphragm-HI.M.Norm-hi BMI-31 | 0.9 |
| 137843_diaphragm-AA.M.Norm-hi BMI-25 | 1.7 |
| 137879_diaphragm-AA.M.Norm-med BMI-47 | 1.7 |
| 137872_diaphragm-CC.M.Norm-med BMI-26 | 1.2 |
| 135773_diaphragm-HI.M.Norm-med BMI-35 | 51.1 |
| 139542_diaphragm-HI.M.Norm-low BMI-41 | 1.9 |
| 137877_diaphragm-CC.M.Norm-low BMI-39 | 1.2 |
| 137874_diaphragm-AS.M.Norm-low BMI-28 | 0.9 |
| 141340_subQadipose-AA.M.Diab.-hi BMI-6 | 15.8 |
| 137836_subQadipose-HI.M.Diab.-hi BMI-21 | 2.1 |
| 135771_subQadipose-AS.M.Diab-hi BMI-9 | 4.0 |
| 141329_subQadipose-AA.M.Diab-medbmi-8 | 3.1 |
| 137862_subQadipose-CC.M.Diab.-med BMI-1 | 3.8 |
| 135762_subQadipose-HI.M.Diab.-med BMI-23 | 1.1 |
| 141338_subQadipose-AS.M.Diab.-low BMI-20 | 1.4 |
| 139547_subQadipose-HI.M.Diab.-low BMI-22 | 4.4 |
| 135757_subQadipose-CC.M.Diab.-low BMI-13 | 2.4 |
| 134832_subQadipose-AA.M.Diab.-low BMI-17 | 2.8 |
| 141332_subQadipose-HI.M.Norm-hi BMI-31 | 4.0 |
| 135767_subQadipose-CC.M.Norm-hi BMI-29 | 0.4 |
| 135765_subQadipose-AS.M.Norm-hi BMI-34 | 0.9 |
| 141339_subQadipose-HI.M.Norm-med BMI-35 | 6.0 |
| 141334_subQadipose-CC.M.Norm-med BMI-26 | 1.5 |
| 139544_subQadipose-AA.M.Norm-med BMI-47 | 3.3 |
| 137875_subQadipose-AA.M.Norm-med BMI-37 | 1.9 |
| 141331_subQadipose-AS.M.Norm-low BMI-28 | 2.1 |
| 137878_subQadipose-HI.M.Norm-low BMI-41 | 2.2 |
| 137876_subQadipose-CC.M.Norm-low BMI-39 | 2.1 |
| 137859_vis.adipose-AA.M.Diab.-hi BMI-6 | 0.7 |
| 135770_vis.adipose-AS.M.Diab-hi BMI-9 | 5.6 |
| 135759_vis.adipose-HI.M.Diab.-hi BMI-21 | 0.9 |
| 143502_vis.adipose-CC.M.Diab.-med BMI-2 | 9.8 |
| 139510_vis.adipose-AA.M.Diab.-med BMI-8 | 1.4 |
| 137861_vis.adipose-CC.M.Diab.-med-1 | 2.2 |
| 137839_vis.adipose-HI.M.Diab.-med BMI-23 | 1.5 |
| 139546_vis.adipose-HI.M.Diab.-low BMI-22 | 7.3 |
| 137831_vis.adipose-CC.M.Diab.-low BMI-13 | 1.6 |
| 139522_vis.adipose-HI.M.Norm-hi BMI-31 | 2.7 |
| 139516_vis.adipose-AS.M.Norm-hi BMI-34 | 33.9 |
| 137846_vis.adipose-CC.M.Norm-hi BMI-29 | 0.2 |
| 137841_vis.adipose-AA.M.Norm-hi BMI-25 | 0.7 |
| 139543_vis.adipose-AA.M.Norm-med BMI-47 | 20.2 |
| 139532_vis.adipose-AA.M.Norm-med BMI-37 | 2.2 |
| 139530_vis.adipose-HI.M.Norm-med BMI-35 | 1.5 |
| 139539_vis.adipose-HI.M.Norm-low BMI-41 | 2.0 |
| 139535_vis.adipose-CC.M.Norm-low BMI-40 | 13.1 |
| 137852_vis.adipose-CC.M.Norm-low BMI-39 | 2.8 |
| 135768_vis.adipose-AS.M.Norm-low BMI-28 | 0.5 |
| 141327_liver-CC.M.Diab.-hi BMI-4 | 1.1 |
| 139514_liver-HI.M.Diab.-hi BMI-21 | 2.0 |
| 139526_liver-CC.M.Diab.-med BMI-2 | 2.2 |
| 139511_liver-AA.M.Diab.-med BMI-8 | 2.0 |
| 137840_liver-HI.M.Diab.-med BMI-23 | 1.1 |
| 137827_liver-CC.M.Diab.-med BMI-1 | 0.8 |
| 137838_liver-HI.M.Diab.-low BMI-22 | 0.4 |
| 135758_liver-CC.M.Diab.-low BMI-13 | 0.6 |
| 139519_liver-CC.M.Norm-hi BMI-29 | 1.9 |
| 139518_liver-AA.M.Norm-hi BMI-25 | 1.9 |
| 137849_liver-AS.M.Norm-hi BMI-34 | 1.1 |
| 137847_liver-HI.M.Norm-hi BMI-31 | 0.5 |
| 142741_liver-AA.M.Norm-med BMI-37 | 1.8 |
| 141341_liver-HI.M.Norm-med BMI-35 | 1.1 |
| 141335_liver-CC.M.Norm-med BMI-26 | 1.3 |
| 139540_liver-HI.M.Norm-low BMI-41 | 7.1 |
| 139534_liver-CC.M.Norm-low BMI-39 | 5.8 |
| 139521_liver-AS.M.Norm-low BMI-28 | 0.8 |
| 141328_pancreas-CC.M.Diab.-hi BMI-4 | 28.9 |
| 139525_pancreas-AS.M.Diab.-hi BMI-9 | 5.1 |
| 137856_pancreas-AA.M.Diab.-hi BMI-6 | 0.3 |
| 137837_pancreas-HI.M.Diab.-hi BMI-21 | 1.3 |
| 141337_pancreas-CC.M.Diab.-med BMI-2 | 4.4 |
| 139527_pancreas-CC.M.Diab.-med BMI-1 | 7.9 |
| 139515_pancreas-HI.M.Diab.-med BMI-23 | 27.0 |
| 139512_pancreas-AA.M.Diab.-med BMI-8 | 40.3 |
| 142739_pancreas-AS.M.Diab.-low BMI-20 | 25.9 |
| 139513_pancreas-CC.M.Diab.-low BMI-13 | 16.8 |
| 142743_pancreas-AA.M.Norm-hi BMI-25 | 11.0 |
| 139523_pancreas-HI.M.Norm-hi BMI-31 | 16.7 |
| 139520_pancreas-CC.M.Norm-hi BMI-29 | 27.9 |
| 142744_pancreas-HI.M.Norm-med BMI-35 | 3.4 |
| 139545_pancreas-AA.M.Norm-med BMI-47 | 27.9 |
| 139531_pancreas-AA.M.Norm-med BMI-37 | 8.1 |
| 137871_pancreas-CC.M.Norm-med BMI-26 | 1.3 |
| 139541_pancreas-Hi.M.Norm-low BMI-41 | 16.5 |
| 139537_pancreas-CC.M.Norm-low BMI-40 | 14.7 |
| 139533_pancreas-CC.M.Norm-low BMI-39 | 2.8 |
| 137845_pancreas-AS.M.Norm-low BMI-28 | 0.9 |
| 143530_small intestine-AA.M.Diab.-hi BMI-6 | 7.7 |
| 143529_small intestine-CC.M.Diab.-hi BMI-4 | 35.1 |
| 143538_small intestine-HI.M.Diab.-med BMI-23 | 55.1 |
| 143531_small intestine-AA.M.Diab.-med BMI-8 | 44.1 |
| 143528_small intestine-CC.M.Diab.-med BMI-2 | 28.7 |
| 143537_small intestine-HI.M.Diab.-low BMI-22 | 17.6 |
| 143535_small intestine-AS.M.Diab.-low BMI-20 | 16.7 |
| 143534_small intestine-AA.M.Diab.-low BMI-17 | 0.8 |
| 143544_small intestine-AS.M.Norm-hi BMI-34 | 16.8 |
| 143543_small intestine-HI.M.Norm-hi BMI-31 | 3.7 |
| 143542_small intestine-CC.M.Norm-hi BMI-29 | 10.3 |
| 143539_small intestine-AA.M.Norm-hi BMI-25 | 31.9 |
| 143548_small intestine-AA.M.Norm-med BMI-47 | 0.0 |
| 143547_small intestine-AA.M.Norm-med BMI-37 | 24.5 |
| 143540_small intestine-CC.M.Norm-med BMI-26 | 3.9 |
| 143550_small intestine-CC.M.Norm-low BMI-40 | 1.4 |
| 143549_small intestine-CC.M.Norm-low BMI-39 | 24.0 |
| 143546_small intestine-HI.M.Norm-low BMI-41 | 4.7 |
| 143525_hypothalamus-HI.M.Diab.-hi BMI-21 | 22.8 |
| 143515_hypothalamus-CC.M.Diab.-hi BMI-4 | 8.4 |
| 143513_hypothalamus-AA.M.Diab.-hi BMI-6 | 1.5 |
| 143507_hypothalamus-AS.M.Diab.-hi BMI-9 | 1.8 |
| 143506_hypothalamus-CC.M.Diab.-med BMI-1 | 3.9 |
| 143505_hypothalamus-HI.M.Diab.-med BMI-23 | 31.0 |
| 143509_hypothalamus-AA.M.Diab.-low BMI-17 | 2.6 |
| 143508_hypothalamus-CC.M.Diab.-low BMI-13 | 2.4 |
| 143503_hypothalamus-AS.M.Diab.-low BMI-20 | 2.7 |
| 143522_hypothalamus-HI.M.Norm-hi BMI-31 | 1.5 |
| 143516_hypothalamus-AS.M.Norm-hi BMI-34 | 1.1 |
| 143511_hypothalamus-CC.M.Norm-hi BMI-29 | 2.4 |
| 143504_hypothalamus-AA.M.Norm-hi BMI-25 | 1.6 |
| 143517_hypothalamus-AA.M.Norm-med BMI-47 | 12.9 |
| 143514_hypothalamus-HI.M.Norm-med BMI-35 | 1.0 |
| 143521_hypothalamus-AS.M.Norm-low BMI-28 | 11.0 |
| 143512_hypothalamus-CC.M.Norm-low BMI-40 | 10.3 |
| 145454_Patient-25pl (CC.Diab.low BMI.no insulin) | 29.1 |
| 110916_Patient-18pl (HI.Diab.obese.no insulin) | 11.5 |
| 110913_Patient-18go (HI.Diab.obese.no insulin) | 15.7 |
| 110911_Patient-17pl (CC.Diab.low BMI.no insulin) | 6.7 |
| 110908_Patient-17go (CC.Diab.low BMI.no insulin) | 14.0 |
| 100752_Patient-15sk (CC.Diab.obese.no insulin) | 5.9 |
| 97828_Patient-13pl (CC.Diab.overwt.no insulin) | 16.6 |
| 160114_Patient 27-ut (CC.Diab.obese.insulin) | 34.4 |
| 160113_Patient 27-pl (CC.Diab.obese.insulin) | 20.2 |
| 160112_Patient 27-sk (CC.Diab.obese.insulin) | 2.9 |
| 160111_Patient 27-go (CC.Diab.obese.insulin) | 11.7 |
| 145461_Patient-26sk (CC.Diab.obese.insulin) | 4.8 |
| 145441_Patient-22sk (CC.Diab.low BMI.insulin) | 3.3 |
| 145438_Patient-22pl (CC.Diab.low BMI.insulin) | 30.6 |
| 145427_Patient-20pl (CC.Diab.overwt.insulin) | 46.7 |
| 97503_Patient-12pl (CC.Diab.unknown | 2.2 |
| BMI.insulin) | |
| 145443_Patient-23pl (CC.Non-diab.overwt) | 34.9 |
| 145435_Patient-21pl (CC.Non-diab.overwt) | 42.0 |
| 110921_Patient-19pl (CC.Non-diab.low BMI) | 16.3 |
| 110918_Patient-19go (CC.Non-diab.low BMI) | 8.5 |
| 97481_Patient-08sk (CC.Non-diab.obese) | 10.4 |
| 97478_Patient-07pl (CC.Non-diab.obese) | 9.2 |
| 160117_Human Islets-male, obese | 0.9 |
| 145474_PANC1 (pancreas carcinoma) 1 | 1.5 |
| 154911_Capan2 (pancreas adenocarcinoma) | 1.2 |
| 141190_SW579 (thyroid carcinoma) | 25.3 |
| 145489_SK-N-MC (neuroblastoma) 1 | 75.8 |
| 145495_SK-N-SH (neuroblastoma) 1 | 34.4 |
| 145498_U87 MG (glioblastoma) 2 | 1.4 |
| 145484_HEp-2 (larynx carcinoma) 1 | 2.6 |
| 145479_A549 (lung carcinoma) | 3.3 |
| 145488_A427 (lung carcinoma) 2 | 2.4 |
| 145472_FHs 738Lu (normal lung) 1 | 59.0 |
| 141187_SKW6.4 (B lymphocytes) | 0.5 |
| 154644_IM-9 (immunoglobulin secreting | 0.3 |
| lymphoblast) | |
| 154645_MOLT-4 (acute lymphoblastic leukemia | 1.3 |
| derived from peripheral blood) | |
| 154648_U-937 (histiocystic lymphoma) | 100.0 |
| 154647_Daudi (Burkitt's lymphoma) | 0.6 |
| 145494_SK-MEL-2 (melanoma) 2 | 11.7 |
| 141176_A375 (melanoma) | 2.9 |
| 154642_SW 1353 (humerus chondrosarcoma) | 13.8 |
| 141179_HT-1080 (fibrosarcoma) | 4.3 |
| 145491_MG-63 (osteosarcoma) 1 | 35.4 |
| 141186_MCF7 (breast carcinoma) | 5.3 |
| 141193_T47D (breast carcinoma) | 1.9 |
| 154641_BT-20 (breast carcinoma) | 0.8 |
| 141175_293 (kidney transformed with adenovirus | 1.3 |
| 5 DNA) | |
| 141182_HUH hepatoma 1 | 2.4 |
| 141184_HUH7 hepatoma 1 | 3.2 |
| 145478_HT1376 (bladder carcinoma) | 0.3 |
| 145481_SCaBER (bladder carcinoma) | 1.0 |
| 141192_SW620 (lymph node metastatsis, colon | 1.8 |
| carcinoma) 2 | |
| 141180_HT29 (colon carcinoma) 1 | 0.5 |
| 141188_SW480 (colon carcinoma) 1 | 0.9 |
| 154646_CAOV-3 (ovary adenocarcinoma) | 1.0 |
| 141194_HeLa (cervix carcinoma)- 2 | 2.2 |
| 145482_HeLa S3 (cervix carcinoma) 1 | 1.4 |
| 145486_DU145 (prostate carcinoma) | 1.1 |
| 154643_PC-3 (prostate adenocarcinoma) | 2.0 |
| 154649_HCT-8 (ileocecal adenocarcinoma) | 5.9 |

Column A - Rel. Exp. (%) Ag1402, Run 390904156

TABLE ID
Panel 1.2

| Tissue Name | A | B |
|---|---|---|
| Endothelial cells | 15.3 | 15.8 |
| Heart (Fetal) | 45.4 | 33.9 |
| Pancreas | 1.3 | 0.5 |
| Pancreatic ca. CAPAN 2 | 0.2 | 0.5 |
| Adrenal Gland | 22.2 | 17.7 |
| Thyroid | 1.5 | 0.5 |
| Salivary gland | 12.0 | 10.4 |
| Pituitary gland | 1.7 | 2.5 |
| Brain (fetal) | 0.2 | 0.3 |
| Brain (whole) | 1.3 | 1.6 |
| Brain (amygdala) | 3.0 | 3.0 |
| Brain (cerebellum) | 2.8 | 0.8 |
| Brain (hippocampus) | 9.5 | 9.5 |
| Brain (thalamus) | 5.8 | 5.6 |
| Cerebral Cortex | 15.4 | 12.3 |
| Spinal cord | 1.5 | 1.1 |
| glio/astro U87-MG | 1.2 | 1.4 |
| glio/astro U-118-MG | 6.7 | 7.7 |
| astrocytoma SW1783 | 1.8 | 2.3 |
| neuro*; met SK-N-AS | 32.1 | 18.9 |
| astrocytoma SF-539 | 43.5 | 30.4 |
| astrocytoma SNB-75 | 2.9 | 2.7 |
| glioma SNB-19 | 1.7 | 1.1 |
| glioma U251 | 7.0 | 5.8 |
| glioma SF-295 | 24.8 | 19.5 |
| Heart | 100.0 | 100.0 |
| Skeletal Muscle | 19.6 | 16.4 |
| Bone marrow | 1.7 | 2.2 |
| Thymus | 1.6 | 0.9 |
| Spleen | 10.2 | 7.6 |
| Lymph node | 1.4 | 1.3 |
| Colorectal Tissue | 1.6 | 4.2 |
| Stomach | 5.1 | 4.7 |
| Small intestine | 40.1 | 35.6 |
| Colon ca. SW480 | 1.2 | 0.5 |
| Colon ca.* SW620 (SW480 met) | 3.1 | 2.0 |
| Colon ca. HT29 | 0.2 | 0.2 |
| Colon ca. HCT-116 | 5.6 | 3.0 |
| Colon ca. CaCo-2 | 1.7 | 0.9 |
| Colon ca. Tissue (ODO3866) | 2.2 | 2.3 |
| Colon ca. HCC-2998 | 8.7 | 4.6 |
| Gastric ca.* (liver met) NCI-N87 | 1.2 | 1.3 |
| Bladder | 20.9 | 18.6 |
| Trachea | 1.4 | 0.8 |
| Kidney | 27.5 | 30.6 |
| Kidney (fetal) | 46.3 | 13.7 |
| Renal ca. 786-0 | 0.5 | 0.4 |
| Renal ca. A498 | 1.6 | 1.4 |
| Renal ca. RXF 393 | 2.5 | 2.3 |
| Renal ca. ACHN | 1.7 | 2.1 |
| Renal ca. UO-31 | 1.6 | 1.1 |
| Renal ca. TK-10 | 3.3 | 3.3 |
| Liver | 13.2 | 12.2 |
| Liver (fetal) | 8.3 | 6.2 |
| Liver ca. (hepatoblast) HepG2 | 2.5 | 0.8 |
| Lung | 1.5 | 1.4 |
| Lung (fetal) | 12.2 | 5.4 |
| Lung ca. (small cell) LX-1 | 3.3 | 1.9 |
| Lung ca. (small cell) NCI-H69 | 3.2 | 2.7 |
| Lung ca. (s. cell var.) SHP-77 | 0.9 | 0.5 |
| Lung ca. (large cell)NCI-H460 | 2.8 | 2.1 |
| Lung ca. (non-sm. cell) A549 | 3.1 | 1.5 |
| Lung ca. (non-s. cell) NCI-H23 | 2.3 | 3.4 |
| Lung ca. (non-s. cell) HOP-62 | 5.3 | 11.7 |
| Lung ca. (non-s. cl) NCI-H522 | 11.3 | 9.3 |
| Lung ca. (squam.) SW 900 | 1.2 | 1.1 |
| Lung ca. (squam.) NCI-H596 | 2.0 | 2.0 |
| Mammary gland | 4.2 | 29.5 |
| Breast ca.* (pl. ef) MCF-7 | 0.6 | 0.7 |
| Breast ca.* (pl. ef) MDA-MB-231 | 0.3 | 0.2 |
| Breast ca.* (pl. ef) T47D | 3.5 | 2.5 |
| Breast ca. BT-549 | 0.4 | 0.4 |
| Breast ca. MDA-N | 12.2 | 14.2 |
| Ovary | 79.6 | 54.3 |
| Ovarian ca. OVCAR-3 | 1.0 | 0.8 |
| Ovarian ca. OVCAR-4 | 1.8 | 0.0 |
| Ovarian ca. OVCAR-5 | 7.6 | 4.5 |
| Ovarian ca. OVCAR-8 | 20.4 | 12.2 |
| Ovarian ca. IGROV-1 | 1.4 | 1.1 |
| Ovarian ca. (ascites) SK-OV-3 | 14.4 | 11.2 |
| Uterus | 34.4 | 19.6 |
| Placenta | 6.7 | 4.8 |
| Prostate | 70.2 | 74.2 |
| Prostate ca.* (bone met) PC-3 | 7.9 | 8.5 |
| Testis | 1.2 | 0.6 |
| Melanoma Hs688(A).T | 15.8 | 15.3 |
| Melanoma* (met) Hs688(B).T | 33.0 | 26.6 |
| Melanoma UACC-62 | 9.9 | 8.8 |
| Melanoma M14 | 3.3 | 2.9 |
| Melanoma LOX IMVI | 0.8 | 1.0 |
| Melanoma* (met) SK-MEL-5 | 5.8 | 6.1 |

Column A - Rel. Exp. (%) Ag1402, Run 138249713
Column B - Rel. Exp. (%) Ag1402, Run 138253129

TABLE IE
Panel 2.2

| Tissue Name | A |
|---|---|
| Normal Colon | 29.9 |
| Colon cancer (OD06064) | 41.2 |
| Colon Margin (OD06064) | 34.2 |
| Colon cancer (OD06159) | 2.6 |
| Colon Margin (OD06159) | 30.1 |
| Colon cancer (OD06297-04) | 7.0 |
| Colon Margin (OD06297-05) | 36.6 |
| CC Gr.2 ascend colon (ODO3921) | 7.2 |
| CC Margin (ODO3921) | 8.7 |
| Colon cancer metastasis (OD06104) | 2.7 |
| Lung Margin (OD06104) | 14.3 |
| Colon mets to lung (OD04451-01) | 1.8 |
| Lung Margin (OD04451-02) | 17.6 |
| Normal Prostate | 86.5 |
| Prostate Cancer (OD04410) | 12.4 |
| Prostate Margin (OD04410) | 32.3 |
| Normal Ovary | 74.7 |
| Ovarian cancer (OD06283-03) | 8.5 |
| Ovarian Margin (OD06283-07) | 34.4 |
| Ovarian Cancer 064008 | 17.3 |
| Ovarian cancer (OD06145) | 17.3 |
| Ovarian Margin (OD06145) | 25.0 |
| Ovarian cancer (OD06455-03) | 6.4 |
| Ovarian Margin (OD06455-07) | 100.0 |
| Normal Lung | 18.9 |
| Invasive poor diff. lung adeno (ODO4945-01 | 7.7 |
| Lung Margin (ODO4945-03) | 20.3 |
| Lung Malignant Cancer (OD03126) | 8.8 |
| Lung Margin (OD03126) | 7.7 |
| Lung Cancer (OD05014A) | 8.8 |
| Lung Margin (OD05014B) | 29.1 |
| Lung cancer (OD06081) | 11.1 |
| Lung Margin (OD06081) | 6.5 |
| Lung Cancer (OD04237-01) | 8.7 |
| Lung Margin (OD04237-02) | 27.7 |
| Ocular Melanoma Metastasis | 3.1 |
| Ocular Melanoma Margin (Liver) | 9.3 |
| Melanoma Metastasis | 10.3 |
| Melanoma Margin (Lung) | 18.2 |
| Normal Kidney | 10.6 |
| Kidney Ca, Nuclear grade 2 (OD04338) | 24.5 |
| Kidney Margin (OD04338) | 4.9 |
| Kidney Ca Nuclear grade 1/2 (OD04339) | 5.2 |
| Kidney Margin (OD04339) | 10.7 |
| Kidney Ca, Clear cell type (OD04340) | 12.9 |
| Kidney Margin (OD04340) | 10.1 |
| Kidney Ca, Nuclear grade 3 (OD04348) | 14.6 |
| Kidney Margin (OD04348) | 32.3 |
| Kidney malignant cancer (OD06204B) | 2.2 |
| Kidney normal adjacent tissue (OD06204E) | 9.1 |
| Kidney Cancer (OD04450-01) | 2.9 |
| Kidney Margin (OD04450-03) | 7.8 |
| Kidney Cancer 8120613 | 1.2 |
| Kidney Margin 8120614 | 9.5 |
| Kidney Cancer 9010320 | 5.8 |
| Kidney Margin 9010321 | 3.7 |
| Kidney Cancer 8120607 | 16.6 |
| Kidney Margin 8120608 | 6.0 |
| Normal Uterus | 88.9 |
| Uterine Cancer 064011 | 14.3 |
| Normal Thyroid | 7.6 |
| Thyroid Cancer 064010 | 2.2 |
| Thyroid Cancer A302152 | 11.6 |
| Thyroid Margin A302153 | 3.9 |
| Normal Breast | 95.3 |
| Breast Cancer (OD04566) | 4.0 |
| Breast Cancer 1024 | 58.6 |
| Breast Cancer (OD04590-01) | 19.3 |
| Breast Cancer Mets (OD04590-03) | 22.7 |
| Breast Cancer Metastasis (OD04655-05) | 12.1 |
| Breast Cancer 064006 | 16.8 |
| Breast Cancer 9100266 | 13.9 |
| Breast Margin 9100265 | 51.4 |
| Breast Cancer A209073 | 13.8 |
| Breast Margin A2090734 | 42.0 |
| Breast cancer (OD06083) | 26.6 |
| Breast cancer node metastasis (OD06083) | 39.5 |
| Normal Liver | 16.4 |
| Liver Cancer 1026 | 11.6 |
| Liver Cancer 1025 | 13.4 |
| Liver Cancer 6004-T | 11.3 |
| Liver Tissue 6004-N | 11.8 |
| Liver Cancer 6005-T | 28.5 |
| Liver Tissue 6005-N | 23.2 |
| Liver Cancer 064003 | 4.3 |
| Normal Bladder | 12.3 |
| Bladder Cancer 1023 | 16.5 |
| Bladder Cancer A302173 | 8.9 |
| Normal Stomach | 47.0 |
| Gastric Cancer 9060397 | 5.8 |
| Stomach Margin 9060396 | 17.6 |
| Gastric Cancer 9060395 | 19.1 |
| Stomach Margin 9060394 | 50.0 |
| Gastric Cancer 064005 | 8.4 |

Column A - Rel. Exp. (%) Ag1402, Run 173860059

TABLE IF
Panel 3D

| Tissue Name | A |
|---|---|
| Daoy- Medulloblastoma | 1.9 |
| TE671- Medulloblastoma | 8.0 |
| D283 Med- Medulloblastoma | 13.0 |
| PFSK-1- Primitive Neuroectodermal | 61.1 |
| XF-498- CNS | 16.0 |
| SNB-78- Glioma | 81.8 |
| SF-268- Glioblastoma | 4.5 |
| T98G- Glioblastoma | 16.3 |
| SK-N-SH- Neuroblastoma (metastasis) | 39.2 |
| SF-295- Glioblastoma | 5.3 |
| Cerebellum | 7.7 |
| Cerebellum | 13.4 |
| NCI-H292- Mucoepidermoid lung carcinoma | 2.1 |
| DMS-114- Small cell lung cancer | 31.2 |
| DMS-79- Small cell lung cancer | 31.9 |
| NCI-H146- Small cell lung cancer | 3.1 |
| NCI-H526- Small cell lung cancer | 9.5 |
| NCI-N417- Small cell lung cancer | 0.2 |
| NCI-H82- Small cell lung cancer | 3.7 |
| NCI-H157- Squamous cell lung cancer (metastasis) | 1.4 |
| NCI-H1155- Large cell lung cancer | 9.2 |
| NCI-H1299- Large cell lung cancer | 1.5 |
| NCI-H727- Lung carcinoid | 1.5 |
| NCI-UMC-11- Lung carcinoid | 2.0 |
| LX-1- Small cell lung cancer | 1.5 |
| Colo-205- Colon cancer | 0.9 |
| KM12- Colon cancer | 0.9 |
| KM20L2- Colon cancer | 0.5 |
| NCI-H716- Colon cancer | 5.6 |
| SW-48- Colon adenocarcinoma | 0.3 |
| SW1116- Colon adenocarcinoma | 5.3 |
| LS 174T- Colon adenocarcinoma | 1.1 |
| SW-948- Colon adenocarcinoma | 0.0 |
| SW-480- Colon adenocarcinoma | 0.4 |
| NCI-SNU-5- Gastric carcinoma | 0.8 |
| KATO III- Gastric carcinoma | 2.4 |
| NCI-SNU-16- Gastric carcinoma | 0.2 |
| NCI-SNU-1- Gastric carcinoma | 1.6 |
| RF-1- Gastric adenocarcinoma | 0.5 |
| RF-48- Gastric adenocarcinoma | 0.9 |
| MKN-45- Gastric carcinoma | 0.7 |
| NCI-N87- Gastric carcinoma | 0.9 |
| OVCAR-5- Ovarian carcinoma | 1.7 |
| RL95-2- Uterine carcinoma | 6.7 |
| HelaS3- Cervical adenocarcinoma | 3.3 |
| Ca Ski- Cervical epidermoid carcinoma (metastasis) | 1.9 |
| ES-2- Ovarian clear cell carcinoma | 3.2 |
| Ramos- Stimulated with PMA/ionomycin 6 h | 0.8 |
| Ramos- Stimulated with PMA/ionomycin 14 h | 0.6 |
| MEG-01- Chronic myelogenous leukemia | 1.0 |
| (megokaryoblast) | |
| Raji- Burkitt's lymphoma | 0.1 |
| Daudi- Burkitt's lymphoma | 1.0 |
| U266- B-cell plasmacytoma | 1.0 |
| CA46- Burkitt's lymphoma | 2.0 |
| RL- non-Hodgkin's B-cell lymphoma | 0.2 |
| JM1- pre-B-cell lymphoma | 2.6 |
| Jurkat- T cell leukemia | 5.1 |
| TF-1- Erythroleukemia | 1.0 |
| HUT 78- T-cell lymphoma | 3.6 |
| U937- Histiocytic lymphoma | 100.0 |
| KU-812- Myelogenous leukemia | 1.5 |
| 769-P- Clear cell renal carcinoma | 0.7 |
| Caki-2- Clear cell renal carcinoma | 0.9 |
| SW 839- Clear cell renal carcinoma | 0.5 |
| Rhabdoid kidney tumor | 34.9 |
| Hs766T- Pancreatic carcinoma (LN metastasis) | 0.9 |
| CAPAN-1- Pancreatic adenocarcinoma (liver | 5.5 |
| metastasis) | |
| SU86.86- Pancreatic carcinoma (liver metastasis) | 1.7 |
| BxPC-3- Pancreatic adenocarcinoma | 0.8 |
| HPAC- Pancreatic adenocarcinoma | 0.2 |
| MIA PaCa-2- Pancreatic carcinoma | 0.0 |
| CFPAC-1- Pancreatic ductal adenocarcinoma | 2.1 |
| PANC-1- Pancreatic epithelioid ductal carcinoma | 0.7 |
| T24- Bladder carcinma (transitional cell) | 1.1 |
| 5637- Bladder carcinoma | 0.9 |
| HT-1197- Bladder carcinoma | 0.6 |
| UM-UC-3- Bladder carcinma (transitional cell) | 0.6 |
| A204- Rhabdomyosarcoma | 12.3 |
| HT-1080- Fibrosarcoma | 7.3 |
| MG-63- Osteosarcoma | 11.9 |
| SK-LMS-1- Leiomyosarcoma (vulva) | 5.0 |
| SJRH30- Rhabdomyosarcoma (met to bone marrow) | 2.3 |
| A431- Epidermoid carcinoma | 1.3 |
| WM266-4- Melanoma | 8.7 |
| DU 145- Prostate carcinoma (brain metastasis) | 0.0 |
| MDA-MB-468- Breast adenocarcinoma | 1.8 |
| SCC-4- Squamous cell carcinoma of tongue | 0.0 |
| SCC-9- Squamous cell carcinoma of tongue | 0.3 |
| SCC-15- Squamous cell carcinoma of tongue | 0.1 |
| CAL 27- Squamous cell carcinoma of tongue | 0.7 |

Column A - Rel. Exp. (%) Ag1402, Run 182113343

TABLE IG
Panel 4D

| Tissue Name | A | B |
|---|---|---|
| Secondary Th1 act | 0.5 | 1.0 |
| Secondary Th2 act | 0.2 | 0.4 |
| Secondary Tr1 act | 0.9 | 0.6 |
| Secondary Th1 rest | 0.3 | 0.1 |
| Secondary Th2 rest | 0.4 | 0.2 |
| Secondary Tr1 rest | 0.3 | 0.2 |
| Primary Th1 act | 1.1 | 0.8 |
| Primary Th2 act | 0.9 | 0.8 |
| Primary Tr1 act | 1.9 | 1.4 |
| Primary Th1 rest | 1.6 | 1.1 |
| Primary Th2 rest | 0.7 | 0.5 |
| Primary Tr1 rest | 1.6 | 1.2 |
| CD45RA CD4 lymphocyte act | 16.7 | 13.4 |
| CD45RO CD4 lymphocyte act | 0.5 | 0.3 |
| CD8 lymphocyte act | 0.4 | 0.3 |
| Secondary CD8 lymphocyte rest | 0.5 | 0.4 |
| Secondary CD8 lymphocyte act | 0.6 | 0.3 |
| CD4 lymphocyte none | 0.5 | 0.4 |
| 2ry Th1/Th2/Tr1_anti-CD95 CH11 | 0.6 | 0.3 |
| LAK cells rest | 0.4 | 0.4 |
| LAK cells IL-2 | 0.5 | 0.4 |
| LAK cells IL-2 + IL-12 | 0.7 | 0.5 |
| LAK cells IL-2 + IFN gamma | 0.9 | 0.5 |
| LAK cells IL-2 + IL-18 | 0.5 | 0.3 |
| LAK cells PMA/ionomycin | 0.1 | 0.1 |
| NK Cells IL-2 rest | 0.6 | 0.5 |
| Two Way MLR 3 day | 0.5 | 0.3 |
| Two Way MLR 5 day | 0.4 | 0.2 |
| Two Way MLR 7 day | 0.3 | 0.3 |
| PBMC rest | 0.9 | 0.6 |
| PBMC PWM | 1.5 | 1.2 |
| PBMC PHA-L | 2.1 | 1.5 |
| Ramos (B cell) none | 0.6 | 1.0 |
| Ramos (B cell) ionomycin | 1.1 | 0.9 |
| B lymphocytes PWM | 1.0 | 0.7 |
| B lymphocytes CD40L and IL-4 | 0.5 | 0.3 |
| EOL-1 dbcAMP | 7.0 | 6.6 |
| EOL-1 dbcAMP PMA/ionomycin | 1.0 | 0.7 |
| Dendritic cells none | 1.0 | 0.5 |
| Dendritic cells LPS | 0.1 | 0.1 |
| Dendritic cells anti-CD40 | 0.5 | 0.9 |
| Monocytes rest | 3.7 | 2.5 |
| Monocytes LPS | 4.4 | 3.7 |
| Macrophages rest | 0.2 | 0.3 |
| Macrophages LPS | 0.3 | 0.2 |
| HUVEC none | 7.6 | 5.5 |
| HUVEC starved | 13.4 | 12.4 |
| HUVEC IL-1beta | 2.6 | 2.2 |
| HUVEC IFN gamma | 20.3 | 18.2 |
| HUVEC TNF alpha + IFN gamma | 1.5 | 1.1 |
| HUVEC TNF alpha + IL4 | 2.4 | 2.4 |
| HUVEC IL-11 | 14.0 | 12.5 |
| Lung Microvascular EC none | 14.0 | 12.0 |
| Lung Microvascular EC TNFalpha + IL-1beta | 7.0 | 7.2 |
| Microvascular Dermal EC none | 39.2 | 40.1 |
| Microsvasular Dermal EC TNFalpha + IL-1beta | 14.4 | 12.4 |
| Bronchial epithelium TNFalpha + IL1beta | 0.5 | 0.5 |
| Small airway epithelium none | 2.1 | 1.5 |
| Small airway epithelium TNFalpha + IL-1beta | 0.2 | 0.3 |
| Coronery artery SMC rest | 18.7 | 17.9 |
| Coronery artery SMC TNFalpha + IL-1beta | 17.4 | 13.6 |
| Astrocytes rest | 0.5 | 0.3 |
| Astrocytes TNFalpha + IL-1beta | 0.6 | 0.6 |
| KU-812 (Basophil) rest | 0.6 | 0.4 |
| KU-812 (Basophil) PMA/ionomycin | 1.0 | 0.6 |
| CCD1106 (Keratinocytes) none | 0.1 | 0.2 |
| CCD1106 (Keratinocytes) TNFalpha + IL-1beta | 0.6 | 0.2 |
| Liver cirrhosis | 4.9 | 5.7 |
| Lupus kidney | 5.3 | 4.9 |
| NCI-H292 none | 0.6 | 0.7 |
| NCI-H292 IL-4 | 0.4 | 0.7 |
| NCI-H292 IL-9 | 1.1 | 0.7 |
| NCI-H292 IL-13 | 0.6 | 0.5 |
| NCI-H292 IFN gamma | 0.7 | 0.5 |
| HPAEC none | 12.0 | 11.7 |
| HPAEC TNF alpha + IL-1 beta | 6.2 | 5.1 |
| Lung fibroblast none | 64.6 | 66.0 |
| Lung fibroblast TNF alpha + IL-1 beta | 26.6 | 24.7 |
| Lung fibroblast IL-4 | 64.6 | 66.9 |
| Lung fibroblast IL-9 | 51.4 | 42.9 |
| Lung fibroblast IL-13 | 100.0 | 100.0 |
| Lung fibroblast IFN gamma | 84.1 | 82.4 |
| Dermal fibroblast CCD1070 rest | 71.2 | 60.7 |
| Dermal fibroblast CCD1070 TNF alpha | 41.8 | 39.0 |
| Dermal fibroblast CCD1070 IL-1 beta | 45.7 | 43.8 |
| Dermal fibroblast IFN gamma | 25.3 | 19.5 |
| Dermal fibroblast IL-4 | 45.7 | 42.0 |
| IBD Colitis 2 | 1.2 | 1.1 |
| IBD Crohn's | 2.0 | 1.6 |
| Colon | 8.4 | 8.2 |
| Lung | 29.9 | 23.7 |
| Thymus | 11.3 | 10.8 |
| Kidney | 7.1 | 5.2 |

Column A - Rel. Exp. (%) Ag1402, Run 139531667
Column B - Rel. Exp. (%) Ag1402, Run 145633378

TABLE IH
Panel 5 Islet

| Tissue Name | A |
|---|---|
| 97457_Patient-02go_adipose | 32.5 |
| 97476_Patient-07sk_skeletal muscle | 23.2 |
| 97477_Patient-07ut_uterus | 27.0 |
| 97478_Patient-07pl_placenta | 62.9 |
| 99167_Bayer Patient 1 | 6.9 |
| 97482_Patient-08ut_uterus | 45.1 |
| 97483_Patient-08pl_placenta | 55.9 |
| 97486_Patient-09sk_skeletal muscle | 6.4 |
| 97487_Patient-09ut_uterus | 68.8 |
| 97488_Patient-09pl_placenta | 42.0 |
| 97492_Patient-10ut_uterus | 100.0 |
| 97493_Patient-10pl_placenta | 51.4 |
| 97495_Patient-11go_adipose | 21.2 |
| 97496_Patient-11sk_skeletal muscle | 11.7 |
| 97497_Patient-11ut_uterus | 61.1 |
| 97498_Patient-11pl_placenta | 22.7 |
| 97500_Patient-12go_adipose | 40.6 |
| 97501_Patient-12sk_skeletal muscle | 22.2 |
| 97502_Patient-12ut_uterus | 73.2 |
| 97503_Patient-12pl_placenta | 22.8 |
| 94721_Donor 2 U - A_MesenchymalStem Cells | 51.8 |
| 94722_Donor 2 U - B_Mesenchymal Stem Cells | 40.9 |
| 94723_Donor 2 U - C_Mesenchymal Stem Cells | 37.9 |
| 94709_Donor 2 AM - A_adipose | 35.8 |
| 94710_Donor 2 AM - B_adipose | 23.3 |
| 94711_Donor 2 AM - C_adipose | 17.1 |
| 94712_Donor 2 AD - A_adipose | 49.3 |
| 94713_Donor 2 AD - B_adipose | 69.3 |
| 94714_Donor 2 AD - C_adipose | 66.9 |
| 94742_Donor 3 U - A_Mesenchymal Stem Cells | 17.0 |
| 94743_Donor 3 U - B_Mesenchymal Stem Cells | 23.5 |
| 94730_Donor 3 AM - A_adipose | 26.2 |
| 94731_Donor 3 AM - B_adipose | 16.2 |
| 94732_Donor 3 AM - C_adipose | 19.9 |
| 94733_Donor 3 AD - A_adipose | 34.4 |
| 94734_Donor 3 AD - B_adipose | 25.9 |
| 94735_Donor 3 AD - C_adipose | 34.2 |
| 77138_Liver_HepG2untreated | 19.5 |
| 73556_Heart_Cardiac stromal cells (primary) | 25.2 |
| 81735_Small Intestine | 20.6 |
| 72409_Kidney_Proximal Convoluted Tubule | 0.6 |
| 82685_Small intestine_Duodenum | 5.3 |
| 90650_Adrenal_Adrenocortical adenoma | 0.2 |
| 72410_Kidney_HRCE | 0.7 |
| 72411_Kidney_HRE | 1.8 |
| 73139_Uterus_Uterine smooth muscle cells | 45.1 |

Column A - Rel. Exp. (%) Ag1402, Run 258861208

HASS Panel v1.0 Summary: Ag1402 Highest expression of this gene was detected in primary melanocytes A5 sample (CT=27). This gene showed wide spread expression in this panel with significant expression in astrocytes, glioma and the cancer cell lines subjected to serum starvation, acidosis and anoxia for different time periods. This expression pattern demonstrates that this gene or its protein product is important for cell survival and proliferation.

Human Metabolic Summary: Ag1402 Highest expression of this gene was detected in histiocystic lymphoma cell line(CT=23.6). This gene showed a ubiquitous expression with high expression in cancer cell lines, and metabolic tissues. Please see panel 1.2 for further discussion on the utility of this gene.

Panel 1.2 Summary: Ag1402 This gene was expressed at highest levels in heart (CT=22-23), prostate, and ovarian tissue and was expressed to a lesser degree across a number of samples in Panel 1.2. This gene was expressed largely in normal tissues when compared to cultured cell lines. Expression of this gene is useful for differentiating between heart, prostate and ovarian tissue from other tissues.

Among other metabolically relevant tissues, this gene was moderately expressed in thyroid, pituitary gland, and pancreas, and was highly expressed in adrenal gland, skeletal muscle and fetal/adult liver. High expression of this gene in insulin-responsive tissues such as skeletal muscle and liver demonstrates that this gene or gene product is an effective drug target for the treatment of Type 2 diabetes. High expression in adrenal gland and heart also shows that this gene or its protein product is an antibody, protein, or small molecule target for the treatment of diseases involving these two tissues.

This gene encodes a putative leucine-rich-repeat (LRR), GPCR-like protein. In Drosophilia, the LRR region of axon guidance proteins has been shown to be critical for function, especially in axon repulsion (Battye R. J. Neurosci. 21: 4290-4298.). The leucine-rich-repeat protein encoded by this gene showed high expression across all brain regions, with expression detected in amygdala, cerebellum, hippocampus, thalamus, cerebral cortex and spinal cord, making it an excellent candidate neuronal guidance protein for axons, dendrites and/or growth cones in general. Therapeutic modulation of the levels of this gene or protein, or possible signaling via this protein, is of utility in enhancing/directing compensatory synaptogenesis and fiber growth in the CNS in response to neuronal death (stroke, head trauma), axon lesion (spinal cord injury), or neurodegeneration (Alzheimer's, Parkinson's, Huntington's, vascular dementia or any neurodegenerative disease). In addition, the this gene showed very high levels of expression in the substantia nigra, where loss of dopaminergic neurons is a hallmark of Parkinson's disease and progressive supranuclear palsy. Therapies based upon this gene or its protein product are useful in the treatment of these diseases.

This protein also contains homology to the GPCR family of receptors. Several neurotransmitter receptors are GPCRs, including the dopamine receptor family, the serotonin receptor family, the GABAB receptor, muscarinic acetylcholine receptors, and others; thus this GPCR represents a novel neurotransmitter receptor. Targeting various neurotransmitter receptors (dopamine, serotonin) has proven to be an effective therapy in psychiatric illnesses such as schizophrenia, bipolar disorder and depression. Furthermore the cerebral cortex and hippocampus are regions of the brain that are known to play critical roles in Alzheimer's disease, seizure disorders, and in the normal process of memory formation. Therapeutic modulation of this gene or its protein product is beneficial in one or more of these diseases, as is stimulation and/or blockade of the receptor coded for by the gene. Levels of this gene were high, however, in areas outside of the central nervous system (such as the heart, ovaries, and prostate), demonstrating the possibility of a wider role in intercellular signaling.

Panel 2.2 Summary: Ag1402 Among the samples on this panel, expression of this gene was highest in a sample of normal ovarian tissue adjacent to an ovarian cancer (CT=28.5). There was also a predominance of this expression pattern in a number of other matched normal tissue samples including lung, colon and kidney. Umatched normal tissue samples, such as breast, uterus, ovary and prostate also showed appreciable expression of this gene. This gene or its protein product are useful in differentiating adjacent normal tissue from corresponding malignant tissue for the above listed tissue types. The therapeutic modulation of the gene or the protein encoded by this gene is of use in the treatment of the above listed malignancies.

Panel 3D Summary: : Ag1402 Expression of this gene was highest in a sample derived from a histiocytic lymphoma cell line (CT=28.2). This gene was also highly expressed in several CNS cancer cell lines and cell lines derived from a variety of sarcomas. The expression of this gene is useful for the differentiation of cell lines derived from sarcomas and CNS cancers vs. other cell lines. Therapeutic modulation of this gene or gene product is of therapeutic benefit in the treatment of sarcomas or CNS cancers.

Panel 4D Summary: This gene was highly expressed in endothelium and fibroblasts and was selectively down regulated in these tissues by treatment with IL-1beta and TNFalpha. This gene encodes a putative LRR-containing GPCR that is down regulated in response to the proinflammatory cytokines IL-1beta and TNFalpha in endothelium and fibroblasts. The natural ligand for this GPCR is induced by treatment with TNF and IL-1 and subsequent ligand binding to this putative GPCR results in feedback inhibition. Other signaling pathways induced by TNF and IL-1 reduce the level of transcription of this gene. Antibodies, small molecule or protein therapeutics which prevent the signaling through this putative GPCR are important in the treatment of diseases such as asthma, emphysema, psoriasis, and arthritis.

Panel 5 Islet Summary: Ag1402 Highest expression of this gene was detected in the uterus of a diabetic patient on insulin (CT=28). This gene showed widespread expression in this panel. This pattern was in agreement with the expression profile in General_screening_panel_v1.2 and demonstrates a role for the gene product in cell survival and proliferation. Please see panel 1.2 for further discussion on the utility of this gene.

J. CG51027-06: Optoid Binding Protein/Cell Adhesion Molecule Precursor

Expression of full-length physical clone CG51027-06 was assessed using the primer-probe set Ag6959, described in Table JA. Results of the RTQ-PCR runs are shown in Table JB.

TABLE JA

Probe Name Ag6959

| | | | | |
|---|---|---|---|---|
| | | | Start | SEQ ID |
| Primers | Sequences | Length | Position | No |

| | | | | |
|---|---|---|---|---|
| Forward | 5′-gtctacctcattgtccacgt-3′ | 20 | 420 | 1294 |
| Probe | TET-5′-ctcgtcgcctgtgatggtgaatga-3′- | 24 | 461 | 1295 |
| | TAMRA | | | |
| Reverse | 5′-caaagcaggttcacattgc-3′ | 19 | 490 | 1296 |

TABLE JB
General_screening_panel_v1.6

| Tissue Name | A |
|---|---|
| Adipose | 0.2 |
| Melanoma* Hs688(A).T | 0.0 |
| Melanoma* Hs688(B).T | 0.1 |
| Melanoma* M14 | 0.1 |
| Melanoma* LOXIMVI | 0.0 |
| Melanoma* SK-MEL-5 | 0.3 |
| Squamous cell carcinoma SCC-4 | 0.0 |
| Testis Pool | 5.8 |
| Prostate ca.* (bone met) PC-3 | 0.0 |
| Prostate Pool | 0.2 |
| Placenta | 0.0 |
| Uterus Pool | 0.0 |
| Ovarian ca. OVCAR-3 | 0.2 |
| Ovarian ca. SK-OV-3 | 0.0 |
| Ovarian ca. OVCAR-4 | 0.0 |
| Ovarian ca. OVCAR-5 | 0.0 |
| Ovarian ca. IGROV-1 | 0.0 |
| Ovarian ca. OVCAR-8 | 0.4 |
| Ovary | 4.5 |
| Breast ca. MCF-7 | 0.0 |
| Breast ca. MDA-MB-231 | 0.0 |
| Breast ca. BT 549 | 0.1 |
| Breast ca. T47D | 0.0 |
| Breast ca. MDA-N | 0.0 |
| Breast Pool | 0.3 |
| Trachea | 0.1 |
| Lung | 0.0 |
| Fetal Lung | 1.6 |
| Lung ca. NCI-N417 | 0.0 |
| Lung ca. LX-1 | 0.0 |
| Lung ca. NCI-H146 | 0.0 |
| Lung ca. SHP-77 | 1.3 |
| Lung ca. A549 | 0.0 |
| Lung ca. NCI-H526 | 0.0 |
| Lung ca. NCI-H23 | 25.0 |
| Lung ca. NCI-H460 | 0.0 |
| Lung ca. HOP-62 | 0.1 |
| Lung ca. NCI-H522 | 21.5 |
| Liver | 0.3 |
| Fetal Liver | 1.0 |
| Liver ca. HepG2 | 0.0 |
| Kidney Pool | 0.1 |
| Fetal Kidney | 2.3 |
| Renal ca. 786-0 | 0.0 |
| Renal ca. A498 | 0.0 |
| Renal ca. ACHN | 0.0 |
| Renal ca. UO-31 | 0.1 |
| Renal ca. TK-10 | 0.0 |
| Bladder | 0.2 |
| Gastric ca. (liver met.) NCI-N87 | 0.0 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 0.6 |
| Colon ca. SW1116 | 0.0 |
| Colon ca. Colo-205 | 0.0 |
| Colon ca. SW-48 | 0.0 |
| Colon Pool | 0.2 |
| Small Intestine Pool | 0.2 |
| Stomach Pool | 0.2 |
| Bone Marrow Pool | 0.0 |
| Fetal Heart | 0.2 |
| Heart Pool | 0.0 |
| Lymph Node Pool | 0.0 |
| Fetal Skeletal Muscle | 0.0 |
| Skeletal Muscle Pool | 0.0 |
| Spleen Pool | 0.2 |
| Thymus Pool | 0.0 |
| CNS cancer (glio/astro) U87-MG | 0.0 |
| CNS cancer (glio/astro) U-118-MG | 0.0 |
| CNS cancer (neuro; met) SK-N-AS | 3.8 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.1 |
| Brain (Amygdala) Pool | 9.3 |
| Brain (cerebellum) | 100.0 |
| Brain (fetal) | 61.1 |
| Brain (Hippocampus) Pool | 9.2 |
| Cerebral Cortex Pool | 25.9 |
| Brain (Substantia nigra) Pool | 18.0 |
| Brain (Thalamus) Pool | 16.3 |
| Brain (whole) | 22.8 |
| Spinal Cord Pool | 18.3 |
| Adrenal Gland | 0.9 |
| Pituitary gland Pool | 0.6 |
| Salivary Gland | 0.0 |
| Thyroid (female) | 0.0 |
| Pancreatic ca. CAPAN2 | 0.0 |
| Pancreas Pool | 0.0 |

Column A - Rel. Exp. (%) Ag6959, Run 278388920

General_screeningpanel_v1.6 Summary: Ag6959 Highest expression of this gene was detected in cerebellum (CT=28.6). This gene showed a highly brain-preferential expression, and was expressed at moderate levels in all brain regions examined. This gene codes for a homolog of rat opioid-binding cell adhesion molecule (OBCAM), which is involved in axonal outgrowth (Hachisuka A, Brain Res Dev Brain Res 122(2):183-91) and may be involved in the synaptic machinery. The limited ability of the CNS to regenerate after injury is the major impediment to the treatment of stroke, spinal cord and head trauma, and neurodegenerative diseases. Modulation of this gene, expressed protein and/or use of antibodies or small molecule drug targeting this gene or gene product is useful in enhancing neuroregeneration in any or all of these clinical conditions.

Moderate expression of this gene was also seen in two lung cancer cell line. Expression of this gene is useful as marker to detect the presence of lung cancer and modulation of this gene or expressed protein is useful in the treatment of lung cancer.

K. CG51027-09: Optiod Bindin Protein/Cell Adhesion Molecule Precursor

Expression of gene CG51027-09 was assessed using the primer-probe sets Ag3090 and Ag3092, described in Tables KA and KB. Results of the RTQ-PCR runs are shown in Tables KC and KD.

TABLE KA

Probe Name Ag3090

| | | | | |
|---|---|---|---|---|
| | | | Start | SEQ ID |
| Primers | Sequences | Length | Position | No |

| | | | | |
|---|---|---|---|---|
| Forward | 5′-atgacagactgctgagcagc-3′ | 20 | 241 | 1297 |
| Probe | TET-5′-aaggcctgaaggtgcagacggag-3′- | 23 | 208 | 1298 |
| | TAMRA | | | |
| Reverse | 5′-cgttggcaaagagaagcat-3′ | 19 | 177 | 1299 |

TABLE KB
Probe Name Ag3092

| | | | | |
|---|---|---|---|---|
| | | | Start | SEQ ID |
| Primers | Sequences | Length | Position | No |

| | | | | |
|---|---|---|---|---|
| Forward | 5′-atgacagactgctgagcagc-3′ | 20 | 241 | 1300 |
| Probe | TET-5′-aaggcctgaaggtgcagacggag-3′- | 23 | 208 | 1301 |
| | TAMRA | | | |
| Reverse | 5′-cgttggcaaagagaagcat-3′ | 19 | 177 | 1302 |

TABLE KC
Panel 1.3D

| Tissue Name | A | B |
|---|---|---|
| Liver adenocarcinoma | 0.3 | 0.0 |
| Pancreas | 0.0 | 0.0 |
| Pancreatic ca. CAPAN 2 | 0.0 | 0.0 |
| Adrenal gland | 1.5 | 0.0 |
| Thyroid | 0.0 | 0.0 |
| Salivary gland | 0.0 | 0.0 |
| Pituitary gland | 3.4 | 0.2 |
| Brain (fetal) | 100.0 | 53.6 |
| Brain (whole) | 29.5 | 51.8 |
| Brain (amygdala) | 31.9 | 51.1 |
| Brain (cerebellum) | 94.0 | 71.7 |
| Brain (hippocampus) | 41.2 | 91.4 |
| Brain (substantia nigra) | 27.0 | 24.7 |
| Brain (thalamus) | 20.3 | 100.0 |
| Cerebral Cortex | 89.5 | 37.4 |
| Spinal cord | 16.4 | 7.9 |
| glio/astro U87-MG | 0.0 | 0.0 |
| glio/astro U-118-MG | 0.0 | 0.0 |
| astrocytoma SW1783 | 0.0 | 0.0 |
| neuro*; met SK-N-AS | 2.2 | 3.2 |
| astrocytoma SF-539 | 0.0 | 0.0 |
| astrocytoma SNB-75 | 1.4 | 0.0 |
| glioma SNB-19 | 0.0 | 0.2 |
| glioma U251 | 0.8 | 0.0 |
| glioma SF-295 | 0.0 | 0.1 |
| Heart (fetal) | 0.0 | 0.1 |
| Heart | 0.0 | 0.0 |
| Skeletal muscle (fetal) | 0.0 | 5.2 |
| Skeletal muscle | 0.2 | 0.0 |
| Bone marrow | 0.0 | 0.0 |
| Thymus | 0.0 | 1.6 |
| Spleen | 0.3 | 1.6 |
| Lymph node | 0.0 | 0.0 |
| Colorectal | 1.7 | 0.1 |
| Stomach | 0.0 | 0.0 |
| Small intestine | 0.5 | 0.0 |
| Colon ca. SW480 | 0.0 | 0.0 |
| Colon ca.* SW620(SW480 met) | 0.4 | 0.0 |
| Colon ca. HT29 | 0.5 | 0.1 |
| Colon ca. HCT-116 | 0.0 | 0.2 |
| Colon ca. CaCo-2 | 0.5 | 0.0 |
| Colon ca. tissue(ODO3866) | 5.8 | 0.9 |
| Colon ca. HCC-2998 | 0.7 | 1.0 |
| Gastric ca.* (liver met) NCI-N87 | 0.3 | 0.0 |
| Bladder | 0.0 | 0.0 |
| Trachea | 0.0 | 0.0 |
| Kidney | 0.0 | 0.0 |
| Kidney (fetal) | 13.3 | 4.4 |
| Renal ca. 786-0 | 0.2 | 0.0 |
| Renal ca. A498 | 0.0 | 0.1 |
| Renal ca. RXF 393 | 0.1 | 0.0 |
| Renal ca. ACHN | 0.0 | 0.0 |
| Renal ca. UO-31 | 0.0 | 0.0 |
| Renal ca. TK-10 | 0.5 | 0.0 |
| Liver | 0.0 | 0.8 |
| Liver (fetal) | 1.2 | 0.0 |
| Liver ca. (hepatoblast) HepG2 | 21.2 | 5.4 |
| Lung | 0.0 | 0.0 |
| Lung (fetal) | 1.8 | 2.1 |
| Lung ca. (small cell) LX-1 | 0.0 | 0.0 |
| Lung ca. (small cell) NCI-H69 | 0.2 | 0.0 |
| Lung ca. (s. cell var.) SHP-77 | 1.6 | 0.0 |
| Lung ca. (large cell)NCI-H460 | 0.0 | 2.0 |
| Lung ca. (non-sm. cell) A549 | 0.8 | 0.0 |
| Lung ca. (non-s. cell) NCI-H23 | 21.3 | 10.2 |
| Lung ca. (non-s. cell) HOP-62 | 0.0 | 0.0 |
| Lung ca. (non-s. cl) NCI-H522 | 27.0 | 3.7 |
| Lung ca. (squam.) SW 900 | 0.0 | 0.1 |
| Lung ca. (squam.) NCI-H596 | 0.0 | 0.0 |
| Mammary gland | 0.0 | 0.0 |
| Breast ca.* (pl. ef) MCF-7 | 0.0 | 0.0 |
| Breast ca.* (pl. ef) MDA-MB-231 | 0.5 | 0.0 |
| Breast ca.* (pl. ef) T47D | 0.0 | 0.0 |
| Breast ca. BT-549 | 0.0 | 0.1 |
| Breast ca. MDA-N | 0.4 | 0.0 |
| Ovary | 9.0 | 2.0 |
| Ovarian ca. OVCAR-3 | 0.0 | 0.0 |
| Ovarian ca. OVCAR-4 | 1.1 | 0.0 |
| Ovarian ca. OVCAR-5 | 0.4 | 0.0 |
| Ovarian ca. OVCAR-8 | 0.5 | 0.4 |
| Ovarian ca. IGROV-1 | 0.0 | 0.1 |
| Ovarian ca.* (ascites) SK-OV-3 | 0.0 | 2.6 |
| Uterus | 0.0 | 0.0 |
| Placenta | 0.0 | 0.0 |
| Prostate | 0.0 | 0.3 |
| Prostate ca.* (bone met)PC-3 | 0.2 | 0.1 |
| Testis | 5.9 | 15.0 |
| Melanoma Hs688(A).T | 0.0 | 0.0 |
| Melanoma* (met) Hs688(B).T | 0.0 | 0.0 |
| Melanoma UACC-62 | 0.0 | 0.1 |
| Melanoma M14 | 0.0 | 0.1 |
| Melanoma LOX IMVI | 0.0 | 0.0 |
| Melanoma* (met) SK-MEL-5 | 0.0 | 0.0 |
| Adipose | 3.1 | 0.8 |

Column A - Rel. Exp. (%) Ag3090, Run 167985244
Column B - Rel. Exp. (%) Ag3092, Run 165673593

TABLE KD
Panel 4D

| Tissue Name | A | B |
|---|---|---|
| Secondary Th1 act | 0.0 | 0.0 |
| Secondary Th2 act | 0.1 | 100.0 |
| Secondary Tr1 act | 0.1 | 0.0 |
| Secondary Th1 rest | 0.0 | 0.0 |
| Secondary Th2 rest | 0.0 | 0.0 |
| Secondary Tr1 rest | 0.0 | 0.0 |
| Primary Th1 act | 0.0 | 6.2 |
| Primary Th2 act | 0.0 | 0.0 |
| Primary Tr1 act | 0.0 | 0.0 |
| Primary Th1 rest | 0.0 | 0.0 |
| Primary Th2 rest | 0.0 | 0.0 |
| Primary Tr1 rest | 0.0 | 0.0 |
| CD45RA CD4 lymphocyte act | 0.0 | 2.9 |
| CD45RO CD4 lymphocyte act | 0.0 | 5.3 |
| CD8 lymphocyte act | 0.0 | 0.0 |
| Secondary CD8 lymphocyte rest | 0.0 | 0.0 |
| Secondary CD8 lymphocyte act | 0.0 | 0.0 |
| CD4 lymphocyte none | 0.0 | 0.0 |
| 2ry Th1/Th2/Tr1_anti-CD95 CH11 | 0.0 | 14.2 |
| LAK cells rest | 0.0 | 0.0 |
| LAK cells IL-2 | 0.0 | 0.0 |
| LAK cells IL-2 + IL-12 | 0.1 | 0.0 |
| LAK cells IL-2 + IFN gamma | 0.0 | 15.2 |
| LAK cells IL-2 + IL-18 | 0.0 | 0.0 |
| LAK cells PMA/ionomycin | 100.0 | 3.6 |
| NK Cells IL-2 rest | 0.0 | 0.0 |
| Two Way MLR 3 day | 0.0 | 0.0 |
| Two Way MLR 5 day | 0.0 | 0.0 |
| Two Way MLR 7 day | 0.0 | 14.6 |
| PBMC rest | 0.0 | 4.2 |
| PBMC PWM | 0.3 | 14.2 |
| PBMC PHA-L | 0.5 | 37.1 |
| Ramos (B cell) none | 0.1 | 0.0 |
| Ramos (B cell) ionomycin | 0.0 | 0.0 |
| B lymphocytes PWM | 0.2 | 2.6 |
| B lymphocytes CD40L and IL-4 | 0.0 | 0.0 |
| EOL-1 dbcAMP | 0.0 | 0.0 |
| EOL-1 dbcAMP PMA/ionomycin | 0.5 | 37.9 |
| Dendritic cells none | 0.0 | 0.0 |
| Dendritic cells LPS | 0.0 | 17.3 |
| Dendritic cells anti-CD40 | 0.0 | 0.0 |
| Monocytes rest | 0.0 | 0.0 |
| Monocytes LPS | 0.0 | 0.0 |
| Macrophages rest | 0.0 | 0.0 |
| Macrophages LPS | 0.0 | 5.0 |
| HUVEC none | 0.0 | 0.0 |
| HUVEC starved | 0.0 | 0.0 |
| HUVEC IL-1beta | 0.0 | 0.0 |
| HUVEC IFN gamma | 0.0 | 0.0 |
| HUVEC TNF alpha + IFN gamma | 0.0 | 0.0 |
| HUVEC TNF alpha + IL4 | 0.0 | 0.0 |
| HUVEC IL-11 | 0.0 | 0.0 |
| Lung Microvascular EC none | 0.0 | 0.0 |
| Lung Microvascular EC TNFalpha + IL-1beta | 0.0 | 4.9 |
| Microvascular Dermal EC none | 0.0 | 0.0 |
| Microsvasular Dermal EC TNFalpha + IL-1beta | 0.0 | 0.0 |
| Bronchial epithelium TNFalpha + IL1beta | 0.0 | 0.0 |
| Small airway epithelium none | 0.0 | 0.0 |
| Small airway epithelium TNFalpha + IL-1beta | 0.0 | 0.0 |
| Coronery artery SMC rest | 0.1 | 24.7 |
| Coronery artery SMC TNFalpha + IL-1beta | 0.0 | 0.0 |
| Astrocytes rest | 0.0 | 0.0 |
| Astrocytes TNFalpha + IL-1 beta | 0.0 | 0.0 |
| KU-812 (Basophil) rest | 0.0 | 25.9 |
| KU-812 (Basophil) PMA/ionomycin | 0.1 | 23.3 |
| CCD1106 (Keratinocytes) none | 0.0 | 0.0 |
| CCD1106 (Keratinocytes) TNFalpha + IL-1beta | 0.0 | 0.0 |
| Liver cirrhosis | 0.6 | 25.3 |
| Lupus kidney | 0.0 | 0.0 |
| NCI-H292 none | 0.0 | 0.0 |
| NCI-H292 IL-4 | 0.0 | 0.0 |
| NCI-H292 IL-9 | 0.0 | 0.0 |
| NCI-H292 IL-13 | 0.0 | 0.0 |
| NCI-H292 IFN gamma | 0.0 | 0.0 |
| HPAEC none | 0.0 | 0.0 |
| HPAEC TNF alpha + IL-1 beta | 0.0 | 2.2 |
| Lung fibroblast none | 0.0 | 0.0 |
| Lung fibroblast TNF alpha + IL-1 beta | 0.0 | 0.0 |
| Lung fibroblast IL-4 | 0.0 | 0.0 |
| Lung fibroblast IL-9 | 0.0 | 0.0 |
| Lung fibroblast IL-13 | 0.0 | 0.0 |
| Lung fibroblast IFN gamma | 0.0 | 0.0 |
| Dermal fibroblast CCD1070 rest | 0.0 | 0.0 |
| Dermal fibroblast CCD1070 TNF alpha | 0.0 | 0.0 |
| Dermal fibroblast CCD1070 IL-1 beta | 0.1 | 0.0 |
| Dermal fibroblast IFN gamma | 0.0 | 0.0 |
| Dermal fibroblast IL-4 | 0.1 | 0.0 |
| IBD Colitis 2 | 0.0 | 0.0 |
| IBD Crohn's | 0.0 | 0.0 |
| Colon | 0.5 | 52.1 |
| Lung | 0.3 | 58.2 |
| Thymus | 0.0 | 0.0 |
| Kidney | 0.0 | 0.0 |

Column A - Rel. Exp. (%) Ag3090, Run 164391872
Column B - Rel. Exp. (%) Ag3092, Run 164399701

Panel 1.3D Summary: Ag3090/Ag3092 Highest expression of this gene was detected in regions of the brain, specifically the thalamus and fetal brain (CTS=29-30). This gene showed a highly brain-preferential expression, and was expressed at high levels in all brain regions examined. The protein encoded by this gene is a homolog of opioid-binding cell adhesion molecule (OBCAM), which is involved in axonal outgrowth and in the synaptic machinery. The limited ability of the CNS to regenerate after injury is the major impediment to the treatment of stroke, spinal cord and head trauma, and neurodegenerative diseases. Selective modulation of this gene or its protein product is useful in enhancing neuroregeneration in any or all of these clinical conditions.

Panel 4D Sumnary: Ag3092 Expression of this gene was limited to a sample derived from secondary Th2 cells (CT=34.2). Therapeutic modulation of this gene or expressed protein is useful in reducing or eliminating the symptoms in patients with diseases related to the functions of Th2 cells, including asthma and allergies.

L. CG51373-08 and CG51373-10 and CG51373-11: Weakly Similar to Neural Cell Adhesion Molecule 1, Large Isoform Precursor

Expression of full-length physical clone CG51373-08 and variants CG51373-10 and CG51373-11 was assessed using the primer-probe sets Ag271 and Ag271b, described in Tables LA and LB. Results of the RTQ-PCR runs are shown in Tables LC, LD, LE, LF and LG.

TABLE LA

Probe Name Ag271

| | | | | |
|---|---|---|---|---|
| | | | Start | SEQ ID |
| Primers | Sequences | Length | Position | No |

| | | | | |
|---|---|---|---|---|
| Forward | 5′-acctggacatagggcgtgtct-3′ | 21 | 690 | 1303 |
| Probe | TET-5′-cgaagcatgaacgaagccatccctag-3′- | 26 | 656 | 1304 |
| | TAMRA | | | |
| Reverse | 5′-tcgatggaagtctccttgcc-3′ | 20 | 635 | 1305 |

TABLE LB

Probe Name Ag271b

| | | | | |
|---|---|---|---|---|
| | | | Start | SEQ ID |
| Primers | Sequences | Length | Position | No |

| | | | | |
|---|---|---|---|---|
| Forward | 5′-caccgtgagccaactgcttat-3′ | 21 | 1722 | 1306 |
| Probe | TET-5′-agacacgccctatgtccaggtccg-3′- | 24 | 1690 | 1307 |
| | TAMRA | | | |
| Reverse | 5′-ttcgttcatgcttcggcaa-3′ | 19 | 1667 | 1308 |

TABLE LC
General_screening_panel_v1.4

| Tissue Name | A | B |
|---|---|---|
| Adipose | 6.4 | 6.4 |
| Melanoma* Hs688(A).T | 29.7 | 28.1 |
| Melanoma* Hs688(B).T | 26.4 | 25.7 |
| Melanoma* M14 | 49.0 | 48.0 |
| Melanoma* LOXIMVI | 28.7 | 31.2 |
| Melanoma* SK-MEL-5 | 9.4 | 11.7 |
| Squamous cell carcinoma SCC-4 | 5.0 | 5.4 |
| Testis Pool | 1.8 | 1.6 |
| Prostate ca.* (bone met) PC-3 | 19.8 | 22.4 |
| Prostate Pool | 2.0 | 2.4 |
| Placenta | 4.5 | 7.7 |
| Uterus Pool | 2.3 | 2.7 |
| Ovarian ca. OVCAR-3 | 45.1 | 54.3 |
| Ovarian ca. SK-OV-3 | 88.9 | 87.1 |
| Ovarian ca. OVCAR-4 | 15.9 | 17.7 |
| Ovarian ca. OVCAR-5 | 29.9 | 29.1 |
| Ovarian ca. IGROV-1 | 54.0 | 50.3 |
| Ovarian ca. OVCAR-8 | 29.9 | 36.9 |
| Ovary | 11.0 | 10.4 |
| Breast ca. MCF-7 | 0.0 | 0.0 |
| Breast ca. MDA-MB-231 | 0.0 | 29.7 |
| Breast ca. BT 549 | 88.3 | 100.0 |
| Breast ca. T47D | 53.2 | 60.3 |
| Breast ca. MDA-N | 13.6 | 17.2 |
| Breast Pool | 7.6 | 9.7 |
| Trachea | 3.5 | 4.7 |
| Lung | 1.1 | 1.8 |
| Fetal Lung | 14.0 | 18.7 |
| Lung ca. NCI-N417 | 3.3 | 2.9 |
| Lung ca. LX-1 | 1.5 | 2.2 |
| Lung ca. NCI-H146 | 0.0 | 0.0 |
| Lung ca. SHP-77 | 0.1 | 0.2 |
| Lung ca. A549 | 8.0 | 9.7 |
| Lung ca. NCI-H526 | 0.9 | 0.6 |
| Lung ca. NCI-H23 | 9.0 | 10.4 |
| Lung ca. NCI-H460 | 15.0 | 19.3 |
| Lung ca. HOP-62 | 15.4 | 20.91 |
| Lung ca. NCI-H522 | 1.8 | 2.0 |
| Liver | 0.2 | 0.2 |
| Fetal Liver | 1.8 | 2.4 |
| Liver ca. HepG2 | 0.0 | 0.0 |
| Kidney Pool | 7.6 | 12.9 |
| Fetal Kidney | 10.2 | 13.8 |
| Renal ca. 786-0 | 62.9 | 77.9 |
| Renal ca. A498 | 21.8 | 22.4 |
| Renal ca. ACHN | 45.4 | 49.3 |
| Renal ca. UO-31 | 37.6 | 32.1 |
| Renal ca. TK-10 | 36.6 | 37.4 |
| Bladder | 4.0 | 6.9 |
| Gastric ca. (liver met.) NCI-N87 | 1.8 | 2.7 |
| Gastric ca. KATO III | 0.0 | 0.0 |
| Colon ca. SW-948 | 4.0 | 4.2 |
| Colon ca. SW480 | 7.5 | 9.1 |
| Colon ca.* (SW480 met) SW620 | 2.7 | 3.2 |
| Colon ca. HT29 | 1.3 | 2.3 |
| Colon ca. HCT-116 | 7.0 | 8.3 |
| Colon ca. CaCo-2 | 13.2 | 17.6 |
| Colon cancer tissue | 6.3 | 6.7 |
| Colon ca. SW1116 | 0.1 | 0.0 |
| Colon ca. Colo-205 | 0.0 | 0.0 |
| Colon ca. SW-48 | 0.5 | 0.5 |
| Colon Pool | 5.9 | 5.6 |
| Small Intestine Pool | 4.2 | 4.1 |
| Stomach Pool | 5.4 | 5.7 |
| Bone Marrow Pool | 3.0 | 3.6 |
| Fetal Heart | 1.7 | 1.7 |
| Heart Pool | 3.1 | 3.1 |
| Lymph Node Pool | 6.8 | 10.5 |
| Fetal Skeletal Muscle | 2.4 | 2.1 |
| Skeletal Muscle Pool | 1.6 | 1.4 |
| Spleen Pool | 2.9 | 3.5 |
| Thymus Pool | 5.6 | 5.8 |
| CMS cancer (glio/astro) U87-MG | 25.0 | 31.0 |
| CNS cancer (glio/astro) U-118-MG | 47.3 | 56.6 |
| CNS cancer (neuro; met) SK-N-AS | 18.8 | 23.2 |
| CNS cancer (astro) SF-539 | 36.9 | 44.8 |
| CNS cancer (astro) SNB-75 | 100.0 | 100.0 |
| CNS cancer (glio) SNB-19 | 28.7 | 57.4 |
| CNS cancer (glio) SF-295 | 64.6 | 79.6 |
| Brain (Amygdala) Pool | 0.4 | 0.3 |
| Brain (cerebellum) | 1.2 | 1.3 |
| Brain (fetal) | 6.9 | 9.0 |
| Brain (Hippocampus) Pool | 0.8 | 0.6 |
| Cerebral Cortex Pool | 0.5 | 0.5 |
| Brain (Substantia nigra) Pool | 0.6 | 0.8 |
| Brain (Thalamus) Pool | 0.9 | 0.8 |
| Brain (whole) | 1.0 | 1.1 |
| Spinal Cord Pool | 0.8 | 0.7 |
| Adrenal Gland | 6.5 | 5.7 |
| Pituitary gland Pool | 0.4 | 0.4 |
| Salivary Gland | 1.5 | 1.4 |
| Thyroid (female) | 1.8 | 1.8 |
| Pancreatic ca. CAPAN2 | 24.8 | 23.2 |
| Pancreas Pool | 7.7 | 9.0 |

Column A - Rel. Exp. (%) Ag271, Run 216607738
Column B - Rel. Exp. (%) Ag271b, Run 216607756

TABLE LD
Panel 1

| Tissue Name | A | B | C |
|---|---|---|---|
| Endothelial cells | 11.0 | 5.0 | 3.9 |
| Endothelial cells (treated) | 3.8 | 4.3 | 4.9 |
| Pancreas | 2.7 | 3.1 | 1.9 |
| Pancreatic ca. CAPAN 2 | 38.2 | 24.0 | 19.8 |
| Adrenal gland | 14.1 | 8.2 | 12.7 |
| Thyroid | 8.6 | 6.1 | 4.5 |
| Salivary gland | 3.5 | 4.1 | 3.3 |
| Pituitary gland | 9.5 | 10.2 | 7.3 |
| Brain (fetal) | 7.4 | 9.0 | 5.3 |
| Brain (whole) | 0.8 | 3.7 | 1.3 |
| Brain (amygdala) | 0.1 | 1.7 | 1.7 |
| Brain (cerebellum) | 4.8 | 10.5 | 2.3 |
| Brain (hippocampus) | 0.2 | 1.2 | 1.4 |
| Brain (substantia nigra) | 0.9 | 3.5 | 2.1 |
| Brain (thalamus) | 0.7 | 2.7 | 3.2 |
| Brain (hypothalamus) | 0.5 | 3.4 | 3.3 |
| Spinal cord | 2.1 | 4.0 | 3.6 |
| glio/astro U87-MG | 30.8 | 24.0 | 27.4 |
| glio/astro U-118-MG | 28.5 | 24.8 | 20.0 |
| astrocytoma SW1783 | 21.0 | 17.1 | 17.0 |
| neuro*; met SK-N-AS | 24.1 | 17.6 | 29.9 |
| astrocytoma SF-539 | 35.6 | 27.4 | 41.2 |
| astrocytoma SNB-75 | 56.3 | 65.1 | 32.8 |
| glioma SNB-19 | 42.6 | 53.6 | 55.9 |
| glioma U251 | 39.8 | 26.8 | 48.3 |
| glioma SF-295 | 43.2 | 33.4 | 31.9 |
| Heart | 15.2 | 4.5 | 6.8 |
| Skeletal muscle | 0.1 | 1.9 | 4.6 |
| Bone marrow | 1.1 | 1.7 | 2.1 |
| Thymus | 28.1 | 18.9 | 10.2 |
| Spleen | 2.5 | 5.1 | 3.2 |
| Lymph node | 6.8 | 6.0 | 2.0 |
| Colon (ascending) | 3.4 | 3.2 | 2.6 |
| Stomach | 11.6 | 12.0 | 3.3 |
| Small intestine | 5.9 | 8.7 | 5.5 |
| Colon ca. SW480 | 0.0 | 1.8 | 5.2 |
| Colon ca.* SW620 (SW480 met) | 1.5 | 2.4 | 3.3 |
| Colon ca. HT29 | 0.1 | 1.7 | 3.2 |
| Colon ca. HCT-116 | 13.0 | 10.4 | 8.9 |
| Colon ca. CaCo-2 | 27.9 | 21.8 | 17.3 |
| Colon ca. HCT-15 | 1.7 | 5.0 | 7.3 |
| Colon ca. HCC-2998 | 0.4 | 1.2 | 2.7 |
| Gastric ca.* (liver met) NCI-N87 | 1.1 | 3.1 | 3.5 |
| Bladder | 30.8 | 15.9 | 19.1 |
| Trachea | 7.2 | 7.0 | 4.5 |
| Kidney | 14.8 | 8.9 | 9.8 |
| Kidney (fetal) | 53.6 | 55.9 | 27.7 |
| Renal ca. 786-0 | 94.6 | 96.6 | 70.2 |
| Renal ca. A498 | 65.5 | 65.5 | 46.3 |
| Renal ca. RXF 393 | 41.8 | 27.7 | 29.1 |
| Renal ca. ACHN | 66.9 | 65.1 | 51.4 |
| Renal ca. UO-31 | 46.0 | 41.8 | 54.0 |
| Renal ca. TK-10 | 57.0 | 56.6 | 52.9 |
| Liver | 2.0 | 3.3 | 4.4 |
| Liver (fetal) | 2.0 | 2.4 | 2.9 |
| Liver ca. (hepatoblast) HepG2 | 0.0 | 0.0 | 0.0 |
| Lung | 27.9 | 8.0 | 5.0 |
| Lung (fetal) | 23.8 | 11.9 | 5.3 |
| Lung ca. (small cell) LX-1 | 0.0 | 1.4 | 3.2 |
| Lung ca. (small cell) NCI-H69 | 2.9 | 4.2 | 8.2 |
| Lung ca. (s. cell var.) SHP-77 | 0.0 | 0.4 | 0.2 |
| Lung ca. (large cell)NCI-H460 | 29.5 | 27.0 | 51.4 |
| Lung ca. (non-sm. cell) A549 | 6.6 | 7.1 | 12.2 |
| Lung ca. (non-s. cell) NCI-H23 | 6.6 | 7.1 | 17.2 |
| Lung ca. (non-s. cell) HOP-62 | 19.1 | 15.8 | 40.9 |
| Lung ca. (non-s. cl) NCI-H522 | 4.3 | 5.4 | 5.0 |
| Lung ca. (squam.) SW 900 | 15.8 | 17.1 | 19.1 |
| Lung ca. (squam.) NCI-H596 | 8.3 | 8.7 | 10.5 |
| Mammary gland | 47.6 | 45.1 | 28.9 |
| Breast ca.* (pl. ef) MCF-7 | 0.0 | 0.0 | 0.0 |
| Breast ca.* (pl. ef) MDA-MB-231 | 17.1 | 15.1 | 18.4 |
| Breast ca.* (pl. ef) T47D | 3.1 | 5.3 | 6.4 |
| Breast ca. BT-549 | 70.7 | 65.1 | 26.1 |
| Breast ca. MDA-N | 43.8 | 25.7 | 35.4 |
| Ovary | 54.0 | 39.5 | 27.9 |
| Ovarian ca. OVCAR-3 | 32.8 | 32.3 | 55.5 |
| Ovarian ca. OVCAR-4 | 39.2 | 33.0 | 46.0 |
| Ovarian ca. OVCAR-5 | 49.3 | 35.6 | 46.7 |
| Ovarian ca. OVCAR-8 | 39.5 | 20.0 | 57.4 |
| Ovarian ca. IGROV-1 | 46.0 | 48.0 | 100.0 |
| Ovarian ca. (ascites) SK-OV-3 | 53.2 | 47.6 | 68.8 |
| Uterus | 36.6 | 9.9 | 9.4 |
| Placenta | 18.2 | 23.8 | 11.7 |
| Prostate | 6.4 | 6.7 | 4.3 |
| Prostate ca.* (bone met) PC-3 | 32.8 | 37.6 | 50.3 |
| Testis | 27.7 | 23.8 | 4.7 |
| Melanoma Hs688(A).T | 21.5 | 23.0 | 13.6 |
| Melanoma* (met) Hs688(B).T | 25.3 | 25.3 | 20.3 |
| Melanoma UACC-62 | 28.1 | 23.0 | 58.2 |
| Melanoma M14 | 34.6 | 36.1 | 22.2 |
| Melanoma LOX IMVI | 100.0 | 100.0 | 12.2 |
| Melanoma* (met) SK-MEL-5 | 12.1 | 10.9 | 14.3 |
| Melanoma SK-MEL-28 | 53.6 | 79.0 | 2.5 |

Column A - Rel. Exp. (%) Ag271, Run 87588589
Column B - Rel. Exp. (%) Ag271, Run 88706798
Column C - Rel. Exp. (%) Ag271b, Run 97805788

TABLE LE
Panel 2.2

| Tissue Name | A |
|---|---|
| Normal Colon | 13.7 |
| Colon cancer (OD06064) | 45.4 |
| Colon Margin (OD06064) | 6.7 |
| Colon cancer (OD06159) | 1.7 |
| Colon Margin (OD06159) | 7.0 |
| Colon cancer (OD06297-04) | 5.3 |
| Colon Margin (OD06297-05) | 7.4 |
| CC Gr.2 ascend colon (ODO3921) | 10.5 |
| CC Margin (ODO3921) | 13.3 |
| Colon cancer metastasis (OD06104) | 1.8 |
| Lung Margin (OD06104) | 3.0 |
| Colon mets to lung (OD04451-01) | 21.0 |
| Lung Margin (OD04451-02) | 10.0 |
| Normal Prostate | 2.0 |
| Prostate Cancer (OD04410) | 3.9 |
| Prostate Margin (OD04410) | 10.3 |
| Normal Ovary | 78.5 |
| Ovarian cancer (OD06283-03) | 14.3 |
| Ovarian Margin (OD06283-07) | 16.3 |
| Ovarian Cancer 064008 | 41.8 |
| Ovarian cancer (OD06145) | 36.6 |
| Ovarian Margin (OD06145) | 33.7 |
| Ovarian cancer (OD06455-03) | 6.0 |
| Ovarian Margin (OD06455-07) | 18.7 |
| Normal Lung | 20.7 |
| Invasive poor diff. lung adeno (ODO4945-01 | 3.1 |
| Lung Margin (ODO4945-03) | 10.6 |
| Lung Malignant Cancer (OD03126) | 5.8 |
| Lung Margin (OD03126) | 10.4 |
| Lung Cancer (OD05014A) | 6.2 |
| Lung Margin (OD05014B) | 11.5 |
| Lung cancer (OD06081) | 5.3 |
| Lung Margin (OD06081) | 4.1 |
| Lung Cancer (OD04237-01) | 3.2 |
| Lung Margin (OD04237-02) | 37.9 |
| Ocular Melanoma Metastasis | 18.8 |
| Ocular Melanoma Margin (Liver) | 8.3 |
| Melanoma Metastasis | 40.1 |
| Melanoma Margin (Lung) | 13.5 |
| Normal Kidney | 16.0 |
| Kidney Ca, Nuclear grade 2 (OD04338) | 57.0 |
| Kidney Margin (OD04338) | 12.9 |
| Kidney Ca Nuclear grade 1/2 (OD04339) | 73.2 |
| Kidney Margin (OD04339) | 14.2 |
| Kidney Ca, Clear cell type (OD04340) | 20.0 |
| Kidney Margin (OD04340) | 35.1 |
| Kidney Ca, Nuclear grade 3 (OD04348) | 25.0 |
| Kidney Margin (OD04348) | 100.0 |
| Kidney malignant cancer (OD06204B) | 6.1 |
| Kidney normal adjacent tissue (OD06204E) | 12.3 |
| Kidney Cancer (OD04450-01) | 68.8 |
| Kidney Margin (OD04450-03) | 24.0 |
| Kidney Cancer 8120613 | 0.0 |
| Kidney Margin 8120614 | 11.7 |
| Kidney Cancer 9010320 | 15.5 |
| Kidney Margin 9010321 | 16.6 |
| Kidney Cancer 8120607 | 70.7 |
| Kidney Margin 8120608 | 11.4 |
| Normal Uterus | 36.6 |
| Uterine Cancer 064011 | 11.7 |
| Normal Thyroid | 1.6 |
| Thyroid Cancer 064010 | 10.2 |
| Thyroid Cancer A302152 | 9.3 |
| Thyroid Margin A302153 | 4.9 |
| Normal Breast | 31.4 |
| Breast Cancer (OD04566) | 3.1 |
| Breast Cancer 1024 | 0.0 |
| Breast Cancer (OD04590-01) | 10.2 |
| Breast Cancer Mets (OD04590-03) | 19.1 |
| Breast Cancer Metastasis (OD04655-05) | 6.0 |
| Breast Cancer 064006 | 14.2 |
| Breast Cancer 9100266 | 12.5 |
| Breast Margin 9100265 | 11.4 |
| Breast Cancer A209073 | 15.7 |
| Breast Margin A2090734 | 48.6 |
| Breast cancer (OD06083) | 14.3 |
| Breast cancer node metastasis (OD06083) | 15.6 |
| Normal Liver | 4.3 |
| Liver Cancer 1026 | 12.3 |
| Liver Cancer 1025 | 12.5 |
| Liver Cancer 6004-T | 12.4 |
| Liver Tissue 6004-N | 3.0 |
| Liver Cancer 6005-T | 21.0 |
| Liver Tissue 6005-N | 26.2 |
| Liver Cancer 064003 | 2.5 |
| Normal Bladder | 9.7 |
| Bladder Cancer 1023 | 8.4 |
| Bladder Cancer A302173 | 11.0 |
| Normal Stomach | 22.8 |
| Gastric Cancer 9060397 | 9.9 |
| Stomach Margin 9060396 | 6.6 |
| Gastric Cancer 9060395 | 11.8 |
| Stomach Margin 9060394 | 23.7 |
| Gastric Cancer 064005 | 6.1 |

Column A - Rel. Axp. (%) Ag271b, Run 175148876

TABLE LF
Panel 3D

| Tissue Name | A |
|---|---|
| Daoy- Medulloblastoma | 9.2 |
| TE671- Medulloblastoma | 3.3 |
| D283 Med- Medulloblastoma | 32.1 |
| PFSK-1- Primitive Neuroectodermal | 9.4 |
| XF-498- CNS | 42.3 |
| SNB-78- Glioma | 22.2 |
| SF-268- Glioblastoma | 17.2 |
| T98G- Glioblastoma | 32.3 |
| SK-N-SH- Neuroblastoma (metastasis) | 20.6 |
| SF-295- Glioblastoma | 12.8 |
| Cerebellum | 1.1 |
| Cerebellum | 1.2 |
| NCI-H292- Mucoepidermoid lung carcinoma | 12.6 |
| DMS-114- Small cell lung cancer | 4.1 |
| DMS-79- Small cell lung cancer | 0.3 |
| NCI-H146- Small cell lung cancer | 0.0 |
| NCI-H526- Small cell lung cancer | 2.8 |
| NCI-N417- Small cell lung cancer | 4.3 |
| NCI-H82- Small cell lung cancer | 6.5 |
| NCI-H157- Squamous cell lung cancer (metastasis) | 19.6 |
| NCI-H1155- Large cell lung cancer | 2.0 |
| NCI-H1299- Large cell lung cancer | 12.5 |
| NCI-H727- Lung carcinoid | 3.0 |
| NCI-UMC-11- Lung carcinoid | 0.0 |
| LX-1- Small cell lung cancer | 1.4 |
| Colo-205- Colon cancer | 0.0 |
| KM12- Colon cancer | 0.0 |
| KM20L2- Colon cancer | 1.5 |
| NCI-H716- Colon cancer | 0.0 |
| SW-48- Colon adenocarcinoma | 1.3 |
| SW1116- Colon adenocarcinoma | 0.0 |
| LS 174T- Colon adenocarcinoma | 0.0 |
| SW-948- Colon adenocarcinoma | 1.0 |
| SW-480- Colon adenocarcinoma | 0.4 |
| NCI-SNU-5- Gastric carcinoma | 4.5 |
| KATO III- Gastric carcinoma | 100.0 |
| NCI-SNU-16- Gastric carcinoma | 15.1 |
| NCI-SNU-1- Gastric carcinoma | 0.0 |
| RF-1- Gastric adenocarcinoma | 0.0 |
| RF-48- Gastric adenocarcinoma | 0.0 |
| MKN-45- Gastric carcinoma | 2.5 |
| NCI-N87- Gastric carcinoma | 1.9 |
| OVCAR-5- Ovarian carcinoma | 2.3 |
| RL95-2- Uterine carcinoma | 6.0 |
| HelaS3- Cervical adenocarcinoma | 4.0 |
| Ca Ski- Cervical epidermoid carcinoma (metastasis) | 11.9 |
| ES-2- Ovarian clear cell carcinoma | 8.8 |
| Ramos- Stimulated with PMA/ionomycin 6 h | 0.0 |
| Ramos- Stimulated with PMA/ionomycin 14 h | 0.0 |
| MEG-01- Chronic myelogenous leukemia (megokaryoblast) | 3.8 |
| Raji- Burkitt's lymphoma | 0.0 |
| Daudi- Burkitt's lymphoma | 0.0 |
| U266- B-cell plasmacytoma | 0.0 |
| CA46- Burkitt's lymphoma | 0.0 |
| RL- non-Hodgkin's B-cell lymphoma | 0.0 |
| JM1- pre-B-cell lymphoma | 0.0 |
| Jurkat- T cell leukemia | 5.6 |
| TF-1- Erythroleukemia | 0.0 |
| HUT 78- T-cell lymphoma | 0.3 |
| U937- Histiocytic lymphoma | 0.0 |
| KU-812- Myelogenous leukemia | 0.2 |
| 769-P- Clear cell renal carcinoma | 42.3 |
| Caki-2- Clear cell renal carcinoma | 26.6 |
| SW 839- Clear cell renal carcinoma | 30.1 |
| Rhabdoid kidney tumor | 11.5 |
| Hs766T- Pancreatic carcinoma (LN metastasis) | 23.7 |
| CAPAN-1- Pancreatic adenocarcinoma (liver metastasis) | 3.8 |
| SU86.86- Pancreatic carcinoma (liver metastasis) | 10.7 |
| BxPC-3- Pancreatic adenocarcinoma | 6.0 |
| HPAC- Pancreatic adenocarcinoma | 2.4 |
| MIA PaCa-2- Pancreatic carcinoma | 5.1 |
| CFPAC-1- Pancreatic ductal adenocarcinoma: | 26.1 |
| PANC-1- Pancreatic epithelioid ductal carcinoma | 21.2 |
| T24- Bladder carcinma (transitional cell) | 15.2 |
| 5637- Bladder carcinoma | 6.3 |
| HT-1197- Bladder carcinoma | 1.0 |
| UM-UC-3- Bladder carcinma (transitional cell) | 3.7 |
| A204- Rhabdomyosarcoma | 6.7 |
| HT-1080- Fibrosarcoma | 25.5 |
| MG-63- Osteosarcoma | 16.5 |
| SK-LMS-1- Leiomyosarcoma (vulva) | 11.2 |
| SJRH30- Rhabdomyosarcoma (met to bone marrow) | 4.7 |
| A431- Epidermoid carcinoma | 4.5 |
| WM266-4- Melanoma | 22.1 |
| DU 145- Prostate carcinoma (brain metastasis) | 1.6 |
| MDA-MB-468- Breast adenocarcinoma | 10.6 |
| SCC-4- Squamous cell carcinoma of tongue | 0.7 |
| SCC-9- Squamous cell carcinoma of tongue | 0.3 |
| SCC-15- Squamous cell carcinoma of tongue | 0.3 |
| CAL 27- Squamous cell carcinoma of tongue | 10.4 |

Column A - Rel. Exp. (%) Ag271b, Run 182114260

TABLE LG
Panel 4.1D

| Tissue Name | A |
|---|---|
| Secondary Th1 act | 0.0 |
| Secondary Th2 act | 0.4 |
| Secondary Tr1 act | 0.3 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.6 |
| Primary Th1 rest | 0.3 |
| Primary Th2 rest | 0.0 |
| Primary Tr1 rest | 0.0 |
| CD45RA CD4 lymphocyte act | 33.9 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.1 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.1 |
| NK Cells IL-2 rest | 0.0 |
| Two Way MLR 3 day | 0.0 |
| Two Way MLR 5 day | 0.1 |
| Two Way MLR 7 day | 0.3 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.3 |
| EOL-1 dbcAMP | 0.3 |
| EOL-1 dbcAMP PMA/ionomycin | 0.0 |
| Dendritic cells none | 0.0 |
| Dendritic cells LPS | 0.6 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 18.2 |
| HUVEC starved | 27.0 |
| HUVEC IL-1 beta | 21.0 |
| HUVEC IFN gamma | 19.2 |
| HUVEC TNF alpha + IFN gamma | 26.6 |
| HUVEC TNF alpha + IL4 | 32.5 |
| HUVEC IL-11 | 11.8 |
| Lung Microvascular EC none | 43.2 |
| Lung Microvascular EC TNFalpha + IL-1beta | 34.4 |
| Microvascular Dermal EC none | 35.8 |
| Microsvasular Dermal EC TNFalpha + IL-1beta | 26.4 |
| Bronchial epithelium TNFalpha + IL1beta | 32.3 |
| Small airway epithelium none | 16.8 |
| Small airway epithelium TNFalpha + IL-1beta | 33.2 |
| Coronery artery SMC rest | 47.3 |
| Coronery artery SMC TNFalpha + IL-1beta | 51.8 |
| Astrocytes rest | 59.0 |
| Astrocytes TNFalpha + IL-1beta | 38.7 |
| KU-812 (Basophil) rest | 0.6 |
| KU-812 (Basophil) PMA/ionomycin | 1.0 |
| CCD1106 (Keratinocytes) none | 36.1 |
| CCD1106 (Keratinocytes) TNFalpha + IL-1beta | 30.6 |
| Liver cirrhosis | 4.4 |
| NCI-H292 none | 12.7 |
| NCI-H292 IL-4 | 17.3 |
| NCI-H292 IL-9 | 17.3 |
| NCI-H292 IL-13 | 17.9 |
| NCI-H292 IFN gamma | 12.8 |
| HPAEC none | 17.0 |
| HPAEC TNF alpha + IL-1 beta | 39.8 |
| Lung fibroblast none | 71.7 |
| Lung fibroblast TNF alpha + IL-1 beta | 55.5 |
| Lung fibroblast IL-4 | 66.4 |
| Lung fibroblast IL-9 | 100.0 |
| Lung fibroblast IL-13 | 52.1 |
| Lung fibroblast IFN gamma | 70.7 |
| Dermal fibroblast CCD1070 rest | 66.0 |
| Dermal fibroblast CCD1070 TNF alpha | 61.1 |
| Dermal fibroblast CCD1070 IL-1 beta | 61.6 |
| Dermal fibroblast IFN gamma | 43.2 |
| Dermal fibroblast IL-4 | 59.0 |
| Dermal Fibroblasts rest | 36.6 |
| Neutrophils TNFa + LPS | 2.0 |
| Neutrophils rest | 6.7 |
| Colon | 4.4 |
| Lung | 20.4 |
| Thymus | 11.6 |
| Kidney | 26.6 |

Column A - Rel. Exp. (%) Ag271b, Run 174261189

General_screeningpanel_v1.4 Summary: Ag271/271b Highest expression of this gene was detected in a sample derived from a brain cancer cell line (SNB-75) (CTs=24). There was substantial expression in a number of brain cancer cell lines, renal cancer cell lines, breast cancer cell lines, ovarian cancer cell lines and melanoma cell lines. The expression of this gene is useful for the differentiation of SNB-75 cells from the other samples in the panel. Therapeutic modulation of this gene or gene product, through the use of small molecule drugs, antibodies or protein therapeutics is beneficial for the treatment of brain, renal, ovarian, breast cancers or melanoma.

This gene also had moderate and widespread expression in metabolic tissues including adipose, adult and fetal heart, adult and fetal skeletal muscle, adrenal, thyroid and pancreas. This gene or gene product is an antibody, protein or small molecule target for the treatment of metabolic and endocrine disease, including obesity and Types 1 and 2 diabetes. This gene was differentially expressed in fetal (CTs=29-30) vs adult liver (CTs=33) and expression of this gene or its protein product is useful differentiate between the two sources of this tissue. The higher levels of expression in fetal liver demonstrate that this gene product is involved in the development of the liver and that this gene or gene product is useful in restoring mass or function to the adult liver.

This panel also showed low but significant levels of expression of this gene in the CNS. Please see panel 1 for a discussion of utility of this gene in the central nervous system.

Panel 1 Summary: Ag271/Ag271b Highest expression of this gene was detected in a number of cancer cell lines, including ovarian cancer, pancreatic cancer, prostate cancer, melanoma, renal carcinoma, and CNS cancers, when compared to normal controls. The data presented in panel 1 showed that this gene was expressed highly in samples derived from cancer cell lines and not highly expressed in samples derived from normal tissues. Within the normal kidney samples there was a consistent difference between adult and fetal tissues. Since cell lines and fetal tissues are more proliferative than normal tissues, this expression profile demonstrates that this gene or its protein product is involved in cell proliferation. Inhibition of expression or function of this gene or gene product is a therapeutic avenue for the treatment of cancer or other disease that involve cell proliferation. Therapeutic targeting of this gene or gene product with a monoclonal antibody, protein therapeutic or small molecule will limit or block the extent of tumor cell migration and invasion and tumor metastasis, particularly in melanomas, prostate cancers, pancreatic cancers, ovarian cancers, renal cell carcinomas and CNS cancers. This gene is also be an effective marker for the diagnosis and detection of a variety of cancers.

This gene was also expressed in all CNS tissues examined, but at lower levels than the relatively high expression in all cancer cell lines examined and fetal kidney. This further demonstrates that this gene or its gene product is involved in the cell cycle or cell proliferation because the majority of cells in the CNS are post-mitotic. This gene is useful in stem cell research or therapy in controlling the transition from stem cell to post-mitotic differentiated cell and is of benefit in any disease associated with neuron loss such as Alzheimer's, Parkinson's, or Huntington's diseases, stroke, head trauma, spinal cord trauma, or spinocerebellar ataxia.

Panel 2.2 Summary: Ag271b Highest expression of this gene was seen in normal kidney. Significant expression was also seen in kidney cancer, melanoma and colon cancer when compared to normal adjacent tissue. Expression of this gene is useful in differentiating between these samples and other samples on this panel and as a marker for these cancers. Therapeutic targeting of this gene or its protein product with a monoclonal antibody, small molecule, or protein therapeutic will limit or block the extent of tumor cell migration and invasion and tumor metastasis in these tumors.

Panel 3D Summary: Ag271b Expression of this gene was highest in a gastric cancer cell line. Expression of this gene was detected in a number of cancer cell lines, a result that is consistent with the expression in Panel 1. This gene plays a role in cell proliferation. Inhibition of the expression or function of this gene or gene product is a therapeutic avenue for the treatment of cancer or other disease that involve cell proliferation.

Panel 4.1D Summary: Ag271b This gene, which encodes the extracellular domain of an immunoglobin domain containing membrane protein, was expressed in panel 4.1D in the following resting and cytokine-activated cells and tissues: HUVEC, lung microvascular endothelial cells, small airway epithelium, coronary artery smooth muscle cells, astrocytes, lung fibroblasts, and dermal fibroblasts. This gene or its gene product is useful as a target for therapeutic antibodies, small molecules or protein drugs which antagonize the function of the Ig domain-containing protein and reduce or eliminate the symptoms in patients with inflammatory diseases and autoimmune diseases, such as multiple sclerosis, chronic obstructive pulmonary disease, asthma, emphysema, rheumatoid arthritis, or psoriasis.

M. CG51622-01: Von Ebner Minor Salivary Gland Protein

Expression of gene CG51622-01 was assessed using the primer-probe set Ag719, described in Table MA. Results of the RTQ-PCR runs are shown in Tables MB and MC.

TABLE MA

Probe Name Ag719

| | | | | |
|---|---|---|---|---|
| | | | Start | SEQ ID |
| Primers | Sequences | Length | Position | No |

| | | | | |
|---|---|---|---|---|
| Forward | 5′-ccaggctgaaggtcatcac-3′ | 19 | 260 | 1309 |
| Probe | TET-5′-ctaacatcctccagctgcaggtgaag-3′- | 26 | 281 | 1310 |
| | TAMRA | | | |
| Reverse | 5′-gactagcagctcctggtcatt-3′ | 21 | 316 | 1311 |

TABLE MB
Panel 1.2

| Tissue Name | A | B | C |
|---|---|---|---|
| Endothelial cells | 0.0 | 0.0 | 0.0 |
| Heart (Fetal) | 0.0 | 0.0 | 0.0 |
| Pancreas | 0.1 | 0.0 | 0.0 |
| Pancreatic ca. CAPAN 2 | 0.0 | 0.0 | 0.0 |
| Adrenal Gland | 0.0 | 0.0 | 0.0 |
| Thyroid | 0.1 | 0.0 | 0.0 |
| Salivary gland | 1.6 | 0.8 | 1.8 |
| Pituitary gland | 0.3 | 0.0 | 0.0 |
| Brain (fetal) | 0.0 | 0.0 | 0.0 |
| Brain (whole) | 0.0 | 0.0 | 0.0 |
| Brain (amygdala) | 0.0 | 0.0 | 0.0 |
| Brain (cerebellum) | 0.0 | 0.0 | 0.0 |
| Brain (hippocampus) | 0.0 | 0.0 | 0.0 |
| Brain (thalamus) | 0.0 | 0.0 | 0.0 |
| Cerebral Cortex | 0.0 | 0.0 | 0.0 |
| Spinal cord | 0.1 | 0.0 | 0.1 |
| glio/astro U87-MG | 0.0 | 0.0 | 0.0 |
| glio/astro U-118-MG | 0.0 | 0.0 | 0.0 |
| astrocytoma SW1783 | 0.0 | 0.0 | 0.0 |
| neuro*; met SK-N-AS | 0.0 | 0.0 | 0.0 |
| astrocytoma SF-539 | 0.0 | 0.0 | 0.0 |
| astrocytoma SNB-75 | 0.0 | 0.0 | 0.0 |
| glioma SNB-19 | 0.0 | 0.0 | 0.0 |
| glioma U251 | 0.0 | 0.0 | 0.0 |
| glioma SF-295 | 0.0 | 0.0 | 0.0 |
| Heart | 0.0 | 0.0 | 0.0 |
| Skeletal Muscle | 0.0 | 0.0 | 0.0 |
| Bone marrow | 0.0 | 0.0 | 0.0 |
| Thymus | 0.0 | 0.0 | 0.0 |
| Spleen | 0.0 | 0.0 | 0.0 |
| Lymph node | 0.3 | 0.1 | 0.2 |
| Colorectal Tissue | 0.0 | 0.0 | 0.0 |
| Stomach | 2.3 | 3.4 | 7.3 |
| Small intestine | 0.0 | 0.0 | 0.0 |
| Colon ca. SW480 | 0.0 | 0.0 | 0.0 |
| Colon ca.* SW620 (SW480 met) | 0.0 | 0.0 | 0.0 |
| Colon ca. HT29 | 0.0 | 0.0 | 0.0 |
| Colon ca. HCT-116 | 0.0 | 0.0 | 0.0 |
| Colon ca. CaCo-2 | 0.0 | 0.0 | 0.0 |
| Colon ca. Tissue (ODO3866) | 0.0 | 0.0 | 0.0 |
| Colon ca. HCC-2998 | 0.0 | 0.0 | 0.0 |
| Gastric ca.* (liver met) NCI-N87 | 0.1 | 0.0 | 0.1 |
| Bladder | 0.0 | 0.0 | 0.0 |
| Trachea | 100.0 | 100.0 | 100.0 |
| Kidney | 0.0 | 0.0 | 0.0 |
| Kidney (fetal) | 0.0 | 0.0 | 0.0 |
| Renal ca. 786-0 | 0.0 | 0.0 | 0.0 |
| Renal ca. A498 | 0.0 | 0.0 | 0.0 |
| Renal ca. RXF 393 | 0.0 | 0.0 | 0.0 |
| Renal ca. ACHN | 0.0 | 0.0 | 0.0 |
| Renal ca. UO-31 | 0.0 | 0.0 | 0.0 |
| Renal ca. TK-10 | 0.0 | 0.0 | 0.0 |
| Liver | 0.0 | 0.0 | 0.0 |
| Liver (fetal) | 0.0 | 0.0 | 0.0 |
| Liver ca. (hepatoblast) HepG2 | 0.0 | 0.0 | 0.0 |
| Lung | 5.2 | 2.3 | 4.9 |
| Lung (fetal) | 1.0 | 0.3 | 0.6 |
| Lung ca. (small cell) LX-1 | 0.0 | 0.0 | 0.0 |
| Lung ca. (small cell) NCI-H69 | 0.0 | 0.0 | 0.0 |
| Lung ca. (s. cell var.) SHP-77 | 0.0 | 0.0 | 0.0 |
| Lung ca. (large cell)NCI-H460 | 0.0 | 0.0 | 0.0 |
| Lung ca. (non-sm. cell) A549 | 0.1 | 0.0 | 0.1 |
| Lung ca. (non-s. cell) NCI-H23 | 0.0 | 0.0 | 0.0 |
| Lung ca. (non-s. cell) HOP-62 | 0.0 | 0.0 | 0.0 |
| Lung ca. (non-s. cl) NCI-H522 | 0.0 | 0.0 | 0.0 |
| Lung ca. (squam.) SW 900 | 0.3 | 0.1 | 0.4 |
| Lung ca. (squam.) NCI-H596 | 0.0 | 0.0 | 0.0 |
| Mammary gland | 0.1 | 0.0 | 0.0 |
| Breast ca.* (pl. ef) MCF-7 | 0.0 | 0.0 | 0.0 |
| Breast ca.* (pl. ef) MDA-MB-231 | 0.0 | 0.0 | 0.0 |
| Breast ca.* (pl. ef) T47D | 0.0 | 0.0 | 0.0 |
| Breast ca. BT-549 | 0.0 | 0.0 | 0.0 |
| Breast ca. MDA-N | 0.0 | 0.0 | 0.0 |
| Ovary | 0.0 | 0.0 | 0.0 |
| Ovarian ca. OVCAR-3 | 0.0 | 0.0 | 0.0 |
| Ovarian ca. OVCAR-4 | 0.0 | 0.0 | 0.0 |
| Ovarian ca. OVCAR-5 | 0.9 | 0.3 | 0.4 |
| Ovarian ca. OVCAR-8 | 0.0 | 0.0 | 0.0 |
| Ovarian ca. IGROV-1 | 0.0 | 0.0 | 0.0 |
| Ovarian ca. (ascites) SK-OV-3 | 0.0 | 0.0 | 0.0 |
| Uterus | 0.0 | 0.0 | 0.0 |
| Placenta | 0.0 | 0.0 | 0.0 |
| Prostate | 0.0 | 0.0 | 0.0 |
| Prostate ca.* (bone met) PC-3 | 0.0 | 0.0 | 0.0 |
| Testis | 0.2 | 0.0 | 0.1 |
| Melanoma Hs688(A).T | 0.0 | 0.0 | 0.0 |
| Melanoma* (met) Hs688(B).T | 0.0 | 0.0 | 0.0 |
| Melanoma UACC-62 | 0.1 | 0.0 | 0.1 |
| Melanoma M14 | 0.0 | 0.0 | 0.0 |
| Melanoma LOX IMVI | 0.0 | 0.0 | 0.0 |
| Melanoma* (met) SK-MEL-5 | 0.0 | 0.0 | 0.0 |

Column A - Rel. Exp. (%) Ag719, Run 115164487
Column B - Rel. Exp. (%) Ag719, Run 123273033
Column C - Rel. Exp. (%) Ag719, Run 124111545

TABLE MC
Panel 4D

| Tissue Name | A | B |
|---|---|---|
| Secondary Th1 act | 0.0 | 0.0 |
| Secondary Th2 act | 0.0 | 0.3 |
| Secondary Tr1 act | 0.0 | 0.0 |
| Secondary Th1 rest | 0.0 | 0.0 |
| Secondary Th2 rest | 0.0 | 0.0 |
| Secondary Tr1 rest | 0.0 | 0.0 |
| Primary Th1 act | 0.5 | 0.0 |
| Primary Th2 act | 0.5 | 0.0 |
| Primary Tr1 act | 0.0 | 0.0 |
| Primary Th1 rest | 0.3 | 0.0 |
| Primary Th2 rest | 0.0 | 0.0 |
| Primary Tr1 rest | 0.0 | 0.0 |
| CD45RA CD4 lymphocyte act | 0.0 | 0.0 |
| CD45RO CD4 lymphocyte act | 0.0 | 0.0 |
| CD8 lymphocyte act | 0.0 | 0.0 |
| Secondary CD8 lymphocyte rest | 0.0 | 0.0 |
| Secondary CD8 lymphocyte act | 0.2 | 0.0 |
| CD4 lymphocyte none | 0.0 | 0.0 |
| 2ry Th1/Th2/TM_anti-CD95 CH11 | 0.0 | 0.0 |
| LAK cells rest | 0.0 | 0.0 |
| LAK cells IL-2 | 0.2 | 0.0 |
| LAK cells IL-2 + IL-12 | 0.0 | 0.0 |
| LAK cells IL-2 + IFN gamma | 0.3 | 0.2 |
| LAK cells IL-2 + IL-18 | 0.0 | 0.0 |
| LAK cells PMA/ionomycin | 0.0 | 0.0 |
| NK Cells IL-2 rest | 0.0 | 0.0 |
| Two Way MLR 3 day | 0.0 | 0.0 |
| Two Way MLR 5 day | 0.0 | 0.0 |
| Two Way MLR 7 day | 0.0 | 0.0 |
| PBMC rest | 0.0 | 0.0 |
| PBMC PWM | 0.0 | 0.0 |
| PBMC PHA-L | 0.0 | 0.0 |
| Ramos (B cell) none | 0.0 | 0.0 |
| Ramos (B cell) ionomycin | 0.0 | 0.0 |
| B lymphocytes PWM | 0.0 | 0.0 |
| B lymphocytes CD40L and IL-4 | 0.0 | 0.0 |
| EOL-1 dbcAMP | 0.0 | 0.0 |
| EOL-1 dbcAMP PMA/ionomycin | 0.0 | 0.0 |
| Dendritic cells none | 0.0 | 0.0 |
| Dendritic cells LPS | 0.0 | 0.0 |
| Dendritic cells anti-CD40 | 0.0 | 0.0 |
| Monocytes rest | 0.0 | 0.0 |
| Monocytes LPS | 0.3 | 0.0 |
| Macrophages rest | 0.0 | 0.0 |
| Macrophages LPS | 0.0 | 0.0 |
| HUVEC none | 0.0 | 0.0 |
| HUVEC starved | 0.0 | 0.0 |
| HUVEC IL-1beta | 0.0 | 0.0 |
| HUVEC IFN gamma | 0.0 | 0.5 |
| HUVEC TNF alpha + IFN gamma | 0.5 | 0.0 |
| HUVEC TNF alpha + IL4 | 0.0 | 0.0 |
| HUVEC IL-11 | 0.0 | 0.0 |
| Lung Microvascular EC none | 0.0 | 0.0 |
| Lung Microvascular EC TNFalpha + IL-1beta | 0.0 | 0.0 |
| Microvascular Dermal EC none | 0.0 | 0.0 |
| Microsvasular Dermal EC TNFalpha + IL-1beta | 0.3 | 0.0 |
| Bronchial epithelium TNFalpha + IL1beta | 0.0 | 0.0 |
| Small airway epithelium none | 0.0 | 0.0 |
| Small airway epithelium TNFalpha + IL-1beta | 0.0 | 0.0 |
| Coronery artery SMC rest | 0.0 | 0.0 |
| Coronery artery SMC TNFalpha + IL-1beta | 0.0 | 0.0 |
| Astrocytes rest | 0.0 | 0.0 |
| Astrocytes TNFalpha + IL-1 beta | 0.5 | 0.0 |
| KU-812 (Basophil) rest | 0.0 | 0.0 |
| KU-812 (Basophil) PMA/ionomycin | 0.0 | 0.0 |
| CCD1106 (Keratinocytes) none | 0.0 | 0.0 |
| CCD1106 (Keratinocytes) TNFalpha + IL-1beta | 0.3 | 0.0 |
| Liver cirrhosis | 1.6 | 1.9 |
| Lupus kidney | 0.3 | 0.0 |
| NCI-H292 none | 88.9 | 86.5 |
| NCI-H292 IL-4 | 64.2 | 56.6 |
| NCI-H292 IL-9 | 68.8 | 55.1 |
| NCI-H292 IL-13 | 47.0 | 53.2 |
| NCI-H292 IFN gamma | 30.6 | 19.2 |
| HPAEC none | 0.0 | 0.0 |
| HPAEC TNF alpha + IL-1 beta | 0.0 | 0.0 |
| Lung fibroblast none | 0.0 | 0.0 |
| Lung fibroblast TNF alpha + IL-1 beta | 0.2 | 0.0 |
| Lung fibroblast IL-4 | 0.0 | 0.0 |
| Lung fibroblast IL-9 | 0.1 | 0.0 |
| Lung fibroblast IL-13 | 0.0 | 0.0 |
| Lung fibroblast IFN gamma | 0.0 | 0.0 |
| Dermal fibroblast CCD1070 rest | 0.0 | 0.0 |
| Dermal fibroblast CCD1070 TNF alpha | 0.0 | 0.0 |
| Dermal fibroblast CCD1070 IL-1 beta | 0.0 | 0.0 |
| Dermal fibroblast IFN gamma | 0.0 | 0.0 |
| Dermal fibroblast IL-4 | 0.0 | 0.0 |
| IBD Colitis 2 | 0.0 | 0.0 |
| IBD Crohn's | 1.3 | 0.4 |
| Colon | 0.3 | 1.4 |
| Lung | 100.0 | 100.0 |
| Thymus | 1.0 | 0.0 |
| Kidney | 0.8 | 0.8 |

Column A - Rel. Exp. (%) Ag719, Run 138167117
Column B - Rel. Exp. (%) Ag719, Run 138273090

Panel 1.2 Summary: Ag719 Highest expression of this gene was detected in the trachea (CTs=18-19). Low to moderate expression was also seen in a few cancer cell lines derived from melanoma, lung, and ovarian cancers. Low expression of this gene is also seen in stomach, pancreas, lung, mammary gland, bladder and spinal cord.

Panel 4D Summary: Ag719 Highest expression of this gene was detected in lung (CTs=28-29). Moderate expression of this gene was also seen in resting and activated NCI-H292.

N. CG51622-03: Von Ebner Minor Salivary Gland Protein

Expression of gene CG51622-03 was assessed using the primer-probe set Ag5814, described in Table NA. Results of the RTQ-PCR runs are shown in Table NB.

TABLE NA

Probe Name Ag5814

| | | | | |
|---|---|---|---|---|
| | | | Start | SEQ ID |
| Primers | Sequences | Length | Position | No |

| | | | | |
|---|---|---|---|---|
| Forward | 5′-ccaggagctgctagtcaaga-3′ | 20 | 358 | 1312 |
| Probe | TET-5′-ctggattcaacacgctctccttcctg-3′- | 26 | 396 | 1313 |
| | TAMRA | | | |
| Reverse | 5′-aggaggttcatgacctgctt-3′ | 20 | 437 | 1314 |

TABLE NB
General_screening_panel_v1.5

| Tissue Name | A |
|---|---|
| Adipose | 0.0 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 0.2 |
| Prostate ca.* (bone met) PC-3 | 0.0 |
| Prostate Pool | 0.1 |
| Placenta | 0.0 |
| Uterus Pool | 0.0 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 4.2 |
| Ovarian ca. IGROV-1 | 0.0 |
| Ovarian ca. OVCAR-8 | 0.0 |
| Ovary | 9.0 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 0.0 |
| Trachea | 100.0 |
| Lung | 0.0 |
| Fetal Lung | 0.0 |
| Lung ca. NCI-N417 | 0.0 |
| Lung ca. LX-1 | 0.0 |
| Lung ca. NCI-H146 | 0.0 |
| Lung ca. SHP-77 | 0.0 |
| Lung ca. A549 | 0.1 |
| Lung ca. NCI-H526 | 0.0 |
| Lung ca. NCI-H23 | 0.0 |
| Lung ca. NCI-H460 | 0.0 |
| Lung ca. HOP-62 | 0.0 |
| Lung ca. NCI-H522 | 0.0 |
| Liver | 0.0 |
| Fetal Liver | 0.0 |
| Liver ca. HepG2 | 0.0 |
| Kidney Pool | 0.0 |
| Fetal Kidney | 0.0 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 0.0 |
| Gastric ca. (liver met.) NCI-N87 | 0.2 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.2 |
| Colon cancer tissue | 0.0 |
| Colon ca. SW1116 | 0.0 |
| Colon ca. Colo-205 | 0.0 |
| Colon ca. SW-48 | 0.0 |
| Colon Pool | 0.0 |
| Small Intestine Pool | 0.2 |
| Stomach Pool | 0.0 |
| Bone Marrow Pool | 0.0 |
| Fetal Heart | 0.0 |
| Heart Pool | 0.0 |
| Lymph Node Pool | 0.0 |
| Fetal Skeletal Muscle | 0.0 |
| Skeletal Muscle Pool | 0.0 |
| Spleen Pool | 0.0 |
| Thymus Pool | 0.0 |
| CNS cancer (glio/astro) U87-MG | 0.0 |
| CNS cancer (glio/astro) U-118-MG | 0.0 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) Pool | 0.0 |
| Brain (cerebellum) | 0.0 |
| Brain (fetal) | 0.0 |
| Brain (Hippocampus) Pool | 0.0 |
| Cerebral Cortex Pool | 0.0 |
| Brain (Substantia nigra) Pool | 0.0 |
| Brain (Thalamus) Pool | 0.0 |
| Brain (whole) | 0.0 |
| Spinal Cord Pool | 0.0 |
| Adrenal Gland | 0.0 |
| Pituitary gland Pool | 0.0 |
| Salivary Gland | 0.3 |
| Thyroid (female) | 0.0 |
| Pancreatic ca. CAPAN2 | 0.2 |
| Pancreas Pool | 0.0 |

Column A - Rel. Exp. (%) Ag5814, Run 244897723

General_screening_panel_v1.5 Summary: Ag5814 High levels of expression of this gene were seen in the trachea (CT=27).

O CG51622-04: Von Ebner Minor Salivary Gland Protein

Expression of gene CG51622-04 was assessed using the primer-probe set Ag5210, described in Table OA. Results of the RTQ-PCR runs are shown in Table OB.

TABLE OA

Probe Name Ag5210

| | | | | |
|---|---|---|---|---|
| | | | Start | SEQ ID |
| Primers | Sequences | Length | Position | No |

| | | | | |
|---|---|---|---|---|
| Forward | 5′-aagaagcatgagacgcagact-3′ | 21 | 87 | 1315 |
| Probe | TET-5′-caaagaactcccagtgcaggacaatg-3′- | 26 | 116 | 1316 |
| | TAMRA | | | |
| Reverse | 5′-actgtcctccactcccatgt-3′ | 20 | 165 | 1317 |

TABLE OB
General_screening_panel_v1.5

| Tissue Name | A |
|---|---|
| Adipose | 0.0 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 2.0 |
| Prostate ca.* (bone met) PC-3 | 0.0 |
| Prostate Pool | 0.0 |
| Placenta | 0.0 |
| Uterus Pool | 0.0 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 7.3 |
| Ovarian ca. IGROV-1 | 0.0 |
| Ovarian ca. OVCAR-8 | 0.0 |
| Ovary | 9.9 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 0.0 |
| Trachea | 100.0 |
| Lung | 0.0 |
| Fetal Lung | 0.5 |
| Lung ca. NCI-N417 | 0.0 |
| Lung ca. LX-1 | 0.0 |
| Lung ca. NCI-H146 | 0.0 |
| Lung ca. SHP-77 | 0.0 |
| Lung ca. A549 | 0.5 |
| Lung ca. NCI-H526 | 0.5 |
| Lung ca. NCI-H23 | 0.0 |
| Lung ca. NCI-H460 | 7.7 |
| Lung ca. HOP-62 | 0.0 |
| Lung ca. NCI-H522 | 0.0 |
| Liver | 0.0 |
| Fetal Liver | 0.0 |
| Liver ca. HepG2 | 0.0 |
| Kidney Pool | 0.0 |
| Fetal Kidney | 0.0 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 0.0 |
| Gastric ca. (liver met.) NCI-N87 | 0.9 |
| Gastric ca. KATO III | 0.2 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 0.0 |
| Colon ca. SW1116 | 0.0 |
| Colon ca. Colo-205 | 0.0 |
| Colon ca. SW-48 | 0.0 |
| Colon Pool | 0.0 |
| Small Intestine Pool | 0.2 |
| Stomach Pool | 0.2 |
| Bone Marrow Pool | 1.3 |
| Fetal Heart | 0.0 |
| Heart Pool | 0.0 |
| Lymph Node Pool | 0.0 |
| Fetal Skeletal Muscle | 0.0 |
| Skeletal Muscle Pool | 0.0 |
| Spleen Pool | 0.0 |
| Thymus Pool | 0.2 |
| CNS cancer (glio/astro) U87-MG | 0.0 |
| CNS cancer (glio/astro) U-118-MG | 0.5 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) Pool | 0.0 |
| Brain (cerebellum) | 0.3 |
| Brain (fetal) | 0.1 |
| Brain (Hippocampus) Pool | 0.2 |
| Cerebral Cortex Pool | 0.3 |
| Brain (Substantia nigra) Pool | 0.2 |
| Brain (Thalamus) Pool | 0.0 |
| Brain (whole) | 0.9 |
| Spinal Cord Pool | 0.0 |
| Adrenal Gland | 0.0 |
| Pituitary gland Pool | 0.0 |
| Salivary Gland | 0.2 |
| Thyroid (female) | 0.0 |
| Pancreatic ca. CAPAN2 | 0.8 |
| Pancreas Pool | 0.2 |

Column A - Rel. Exp. (%) Ag5210, Run 228727456

General_screening_panel_v1.5 Summary: Ag5210 Moderate expression was seen in the trachea (CT=30).

P. CG51622-05: Von Ebner Minor Salivary Gland Protein

Expression of gene CG51622-05 was assessed using the primer-probe set Ag5103, described in Table PA. Results of the RTQ-PCR runs are shown in Tables PB, PC, PD and PE.

TABLE PA

| Probe Name Ag5103 | | | | |
|---|---|---|---|---|
| | | | Start | SEQ ID |
| Primers | Sequences | Length | Position | No |
| Forward | 5′-agtgaaaaaccagctggtgaa-3′ | 21 | 661 | 1318 |
| Probe | TET-5′-catttccctcagcattgaccgtctg-3′- | 25 | 688 | 1319 |
| | TAMRA | | | |
| Reverse | 5′-ggcaggatacagaaggtcaaa-3′ | 21 | 716 | 1320 |

TABLE PB
AI_comprehensive panel_v1.0

| Tissue Name | A |
|---|---|
| 110967 COPD-F | 0.0 |
| 110980 COPD-F | 0.0 |
| 110968 COPD-M | 0.0 |
| 110977 COPD-M | 0.0 |
| 110989 Emphysema-F | 0.0 |
| 110992 Emphysema-F | 0.6 |
| 110993 Emphysema-F | 0.0 |
| 110994 Emphysema-F | 0.0 |
| 110995 Emphysema-F | 0.0 |
| 110996 Emphysema-F | 0.0 |
| 110997 Asthma-M | 9.2 |
| 111001 Asthma-F | 0.0 |
| 111002 Asthma-F | 0.0 |
| 111003 Atopic Asthma-F | 0.0 |
| 111004 Atopic Asthma-F | 0.0 |
| 111005 Atopic Asthma-F | 0.0 |
| 111006 Atopic Asthma-F | 0.0 |
| 111417 Allergy-M | 0.0 |
| 112347 Allergy-M | 0.0 |
| 112349 Normal Lung-F | 0.0 |
| 112357 Normal Lung-F | 0.0 |
| 112354 Normal Lung-M | 0.0 |
| 112374 Crohns-F | 0.0 |
| 112389 Match Control Crohns-F | 0.0 |
| 112375 Crohns-F | 0.0 |
| 112732 Match Control Crohns-F | 0.0 |
| 112725 Crohns-M | 0.0 |
| 112387 Match Control Crohns-M | 0.0 |
| 112378 Crohns-M | 0.0 |
| 112390 Match Control Crohns-M | 0.0 |
| 112726 Crohns-M | 0.0 |
| 112731 Match Control Crohns-M | 6.3 |
| 112380 Ulcer Col-F | 0.0 |
| 112734 Match Control Ulcer Col-F | 54.0 |
| 112384 Ulcer Col-F | 0.0 |
| 112737 Match Control Ulcer Col-F | 0.0 |
| 112386 Ulcer Col-F | 0.0 |
| 112738 Match Control Ulcer Col-F | 0.0 |
| 112381 Ulcer Col-M | 0.0 |
| 112735 Match Control Ulcer Col-M | 0.0 |
| 112382 Ulcer Col-M | 0.0 |
| 112394 Match Control Ulcer Col-M | 0.0 |
| 112383 Ulcer Col-M | 0.0 |
| 112736 Match Control Ulcer Col-M | 0.0 |
| 112423 Psoriasis-F | 0.0 |
| 112427 Match Control Psoriasis-F | 0.0 |
| 112418 Psoriasis-M | 0.0 |
| 112723 Match Control Psoriasis-M | 0.0 |
| 112419 Psoriasis-M | 0.0 |
| 112424 Match Control Psoriasis-M | 0.0 |
| 112420 Psoriasis-M | 0.0 |
| 112425 Match Control Psoriasis-M | 0.0 |
| 104689 (MF) OA Bone-Backus | 0.0 |
| 104690 (MF) Adj “Normal” Bone-Backus | 0.0 |
| 104691 (MF) OA Synovium-Backus | 0.0 |
| 104692 (BA) OA Cartilage-Backus | 0.0 |
| 104694 (BA) OA Bone-Backus | 0.0 |
| 104695 (BA) Adj “Normal” Bone-Backus | 0.0 |
| 104696 (BA) OA Synovium-Backus | 0.0 |
| 104700 (SS) OA Bone-Backus | 0.0 |
| 104701 (SS) Adj “Normal” Bone-Backus | 0.0 |
| 104702 (SS) OA Synovium-Backus | 0.0 |
| 117093 OA Cartilage Rep7 | 0.0 |
| 112672 OA Bone5 | 0.0 |
| 112673 OA Synovium5 | 0.0 |
| 112674 OA Synovial Fluid cells5 | 0.0 |
| 117100 OA Cartilage Rep14 | 0.0 |
| 112756 OA Bone9 | 0.0 |
| 112757 OA Synovium9 | 0.0 |
| 112758 OA Synovial Fluid Cells9 | 0.0 |
| 117125 RA Cartilage Rep2 | 0.0 |
| 113492 Bone2 RA | 100.0 |
| 113493 Synovium2 RA | 35.6 |
| 113494 Syn Fluid Cells RA | 53.6 |
| 113499 Cartilage4 RA | 4.0 |
| 113500 Bone4 RA | 2.7 |
| 113501 Synovium4 RA | 1.0 |
| 113502 Syn Fluid Cells4 RA | 0.9 |
| 113495 Cartilage3 RA | 6.0 |
| 113496 Bone3 RA | 3.2 |
| 113497 Synovium3 RA | 4.4 |
| 113498 Syn Fluid Cells3 RA | 6.3 |
| 117106 Normal Cartilage Rep20 | 0.0 |
| 113663 Bone3 Normal | 0.0 |
| 113664 Synovium3 Normal | 0.0 |
| 113665 Syn Fluid Cells3 Normal | 0.0 |
| 117107 Normal Cartilage Rep22 | 0.0 |
| 113667 Bone4 Normal | 0.0 |
| 113668 Synovium4 Normal | 0.0 |
| 113669 Syn Fluid Cells4 Normal | 0.0 |

Column A - Rel. Exp. (%) Ag5103, Run 229314009

TABLE PC
General_screening_panel_v1.5

| Tissue Name | A |
|---|---|
| Adipose | 0.0 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 0.0 |
| Prostate ca.* (bone met) PC-3 | 0.0 |
| Prostate Pool | 0.0 |
| Placenta | 0.0 |
| Uterus Pool | 0.0 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 0.5 |
| Ovarian ca. IGROV-1 | 0.0 |
| Ovarian ca. OVCAR-8 | 0.0 |
| Ovary | 3.6 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 0.0 |
| Trachea | 100.0 |
| Lung | 0.0 |
| Fetal Lung | 0.2 |
| Lung ca. NCI-N417 | 0.0 |
| Lung ca. LX-1 | 0.0 |
| Lung ca. NCI-H146 | 0.0 |
| Lung ca. SHP-77 | 0.0 |
| Lung ca. A549 | 0.0 |
| Lung ca. NCI-H526 | 0.0 |
| Lung ca. NCI-H23 | 0.0 |
| Lung ca. NCI-H460 | 0.0 |
| Lung ca. HOP-62 | 0.0 |
| Lung ca. NCI-H522 | 0.0 |
| Liver | 0.0 |
| Fetal Liver | 0.0 |
| Liver ca. HepG2 | 0.0 |
| Kidney Pool | 0.0 |
| Fetal Kidney | 0.0 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 0.0 |
| Gastric ca. (liver met.) NCI-N87 | 0.1 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 0.0 |
| Colon ca. SW1116 | 0.0 |
| Colon ca. Colo-205 | 0.0 |
| Colon ca. SW-48 | 0.0 |
| Colon Pool | 0.0 |
| Small Intestine Pool | 0.0 |
| Stomach Pool | 0.0 |
| Bone Marrow Pool | 0.1 |
| Fetal Heart | 0.0 |
| Heart Pool | 0.0 |
| Lymph Node Pool | 0.0 |
| Fetal Skeletal Muscle | 0.0 |
| Skeletal Muscle Pool | 0.0 |
| Spleen Pool | 0.0 |
| Thymus Pool | 0.0 |
| CNS cancer (glio/astro) U87-MG | 0.0 |
| CNS cancer (glio/astro) U-118-MG | 0.0 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) Pool | 0.0 |
| Brain (cerebellum) | 0.0 |
| Brain (fetal) | 0.0 |
| Brain (Hippocampus) Pool | 0.0 |
| Cerebral Cortex Pool | 0.0 |
| Brain (Substantia nigra) Pool | 0.0 |
| Brain (Thalamus) Pool | 0.0 |
| Brain (whole) | 0.0 |
| Spinal Cord Pool | 0.0 |
| Adrenal Gland | 0.0 |
| Pituitary gland Pool | 0.0 |
| Salivary Gland | 0.2 |
| Thyroid (female) | 0.0 |
| Pancreatic ca. CAPAN2 | 0.0 |
| Pancreas Pool | 0.0 |

Column A - Rel. Exp. (%) Ag5103, Run 228753956

TABLE PD
Panel 4.1D

| Tissue Name | A |
|---|---|
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.9 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL-1beta | 0.0 |
| Liver cirrhosis | 0.0 |
| NCI-H292 none | 14.7 |
| NCI-H292 IL-4 | 23.0 |
| NCI-H292 IL-9 | 33.2 |
| NCI-H292 IL-13 | 13.2 |
| NCI-H292 IFN gamma | 9.6 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 100.0 |
| Dermal fibroblast IL-4 | 0.0 |
| Dermal Fibroblasts rest | 0.0 |
| Neutrophils TNFa + LPS | 0.0 |
| Neutrophils rest | 0.0 |
| Colon | 2.0 |
| Lung | 24.7 |
| Thymus | 0.3 |
| Kidney | 0.0 |

Column A - Rel. Exp. (%) Ag5103, Run 226160624

TABLE PE
general oncology screening panel_v_2.4

| Tissue Name | A |
|---|---|
| Colon cancer 1 | 0.0 |
| Colon cancer NAT 1 | 0.0 |
| Colon cancer 2 | 0.0 |
| Colon cancer NAT 2 | 0.0 |
| Colon cancer 3 | 0.0 |
| Colon cancer NAT 3 | 0.0 |
| Colon malignant cancer 4 | 0.0 |
| Colon normal adjacent tissue 4 | 0.0 |
| Lung cancer 1 | 6.6 |
| Lung NAT 1 | 19.8 |
| Lung cancer 2 | 0.8 |
| Lung NAT 2 | 0.0 |
| Squamous cell carcinoma 3 | 16.0 |
| Lung NAT 3 | 59.0 |
| metastatic melanoma 1 | 0.0 |
| Melanoma 2 | 0.0 |
| Melanoma 3 | 0.0 |
| metastatic melanoma 4 | 8.0 |
| metastatic melanoma 5 | 100.0 |
| Bladder cancer 1 | 0.0 |
| Bladder cancer NAT 1 | 0.0 |
| Bladder cancer 2 | 0.0 |
| Bladder cancer NAT 2 | 0.0 |
| Bladder cancer NAT 3 | 0.2 |
| Bladder cancer NAT 4 | 0.0 |
| Prostate adenocarcinoma 1 | 0.0 |
| Prostate adenocarcinoma 2 | 0.0 |
| Prostate adenocarcinoma 3 | 0.0 |
| Prostate adenocarcinoma 4 | 0.4 |
| Prostate cancer NAT 5 | 0.0 |
| Prostate adenocarcinoma 6 | 0.0 |
| Prostate adenocarcinoma 7 | 0.0 |
| Prostate adenocarcinoma 8 | 0.5 |
| Prostate adenocarcinoma 9 | 2.0 |
| Prostate cancer NAT 10 | 0.0 |
| Kidney cancer 1 | 0.0 |
| KidneyNAT 1 | 0.0 |
| Kidney cancer 2 | 0.0 |
| Kidney NAT 2 | 0.0 |
| Kidney cancer 3 | 0.0 |
| Kidney NAT 3 | 0.0 |
| Kidney cancer 4 | 0.0 |
| Kidney NAT 4 | 0.0 |

Column A- Rel. Exp. (%) Ag5103, Run259939204

AI_comprehensive panel_v1.0 Summary: Ag5103 Moderate levels of expression were seen in three RA samples, with highest expression in a bone sample from an RA patient (CT=30). Therapeutics targeting of this gene or its protein product is useful for the reduction and inhibition of the inflammation in RA.

General_screening_panel_v1.5 Summary: Ag5103 Extremely high levels of expression were seen in the trachea (CT=23).

Panel 4.1D Summary: AG5103 Highest expression of this gene was detected in IFN gamma treated dermal fibroblasts (CT=31.6). Prominent expression was also seen in a cluster of treated and untreated NCI-H292 cells, a mucoepidermoid cell line. In comparison, expression in the normal lung was relatively low. The expression of the transcript in a cell line that is often used as a model for airway epithelium (NCI-H292 cells) demonstrates that this transcript is important in the proliferation or activation of airway epithelium. Therapeutics designed with the protein encoded for by the transcript are important in the treatment of diseases which include lung airway inflammation such as asthma and COPD.

general oncology screening panel_v_{—}2.4 Summary: Ag5103 Highest expression was seen in a melanoma sample (CT=31). Prominent expression was also seen in two samples derived from normal lung tissue.

Q. CG51821-01: Sialoadhesin

Expression of gene CG51821-01 was assessed using the primer-probe set Ag274, described in Table QA. Results of the RTQ-PCR runs are shown in Tables QB, QC, QD, QE, QF and QG.

TABLE QA
Probe Name Ag274

| | | | | |
|---|---|---|---|---|
| Primers | Sequences | Length | Start Position | SEQ ID No |

| | | | | |
|---|---|---|---|---|
| Forward | 5′-cagaacagatgtattccccttggt-3′ | 24 | 4835 | 1321 |
| Probe | TET-5′-ctcagcgcctcgatgtccaccc-3′-TAMRA | 22 | 4805 | 1322 |
| Reverse | 5′-ctggcttcccccaatgc-3′ | 17 | 4783 | 1323 |

TABLE QB
AI_comprehensive panel_v1.0

| Tissue Name | A |
|---|---|
| 110967 COPD-F | 4.3 |
| 110980 COPD-F | 1.6 |
| 110968 COPD-M | 5.2 |
| 110977 COPD-M | 2.7 |
| 110989 Emphysema-F | 2.2 |
| 110992 Emphysema-F | 0.5 |
| 110993 Emphysema-F | 4.0 |
| 110994 Emphysema-F | 4.3 |
| 110995 Emphysema-F | 5.7 |
| 110996 Emphysema-F | 2.0 |
| 110997 Asthma-M | 1.6 |
| 111001 Asthma-F | 2.2 |
| 111002 Asthma-F | 4.4 |
| 111003 Atopic Asthma-F | 4.0 |
| 111004 Atopic Asthma-F | 3.6 |
| 111005 Atopic Asthma-F | 2.4 |
| 111006 Atopic Asthma-F | 0.5 |
| 111417 Allergy-M | 2.1 |
| 112347 Allergy-M | 0.1 |
| 112349 Normal Lung-F | 0.0 |
| 112357 Normal Lung-F | 8.4 |
| 112354 Normal Lung-M | 2.6 |
| 112374 Crohns-F | 4.4 |
| 112389 Match Control Crohns-F | 2.2 |
| 112375 Crohns-F | 3.9 |
| 112732 Match Control Crohns-F | 3.4 |
| 112725 Crohns-M | 0.6 |
| 112387 Match Control Crohns-M | 4.3 |
| 112378 Crohns-M | 0.2 |
| 112390 Match Control Crohns-M | 2.3 |
| 112726 Crohns-M | 3.5 |
| 112731 Match Control Crohns-M | 3.1 |
| 112380 Ulcer Col-F | 1.1 |
| 112734 Match Control Ulcer Col-F | 8.8 |
| 112384 Ulcer Col-F | 4.9 |
| 112737 Match Control Ulcer Col-F | 0.4 |
| 112386 Ulcer Col-F | 8.4 |
| 112738 Match Control Ulcer Col-F | 6.3 |
| 112381 Ulcer Col-M | 0.5 |
| 112735 Match Control Ulcer Col-M | 0.9 |
| 112382 Ulcer Col-M | 3.3 |
| 112394 Match Control Ulcer Col-M | 2.8 |
| 112383 Ulcer Col-M | 5.3 |
| 112736 Match Control Ulcer Col-M | 5.5 |
| 112423 Psoriasis-F | 2.2 |
| 112427 Match Control Psoriasis-F | 1.8 |
| 112418 Psoriasis-M | 7.8 |
| 112723 Match Control Psoriasis-M | 3.3 |
| 112419 Psoriasis-M | 4.4 |
| 112424 Match Control Psoriasis-M | 1.1 |
| 112420 Psoriasis-M | 6.2 |
| 112425 Match Control Psoriasis-M | 1.5 |
| 104689 (MF) OA Bone-Backus | 28.5 |
| 104690 (MF) Adj “Normal” Bone-Backus | 19.6 |
| 104691 (MF) OA Synovium-Backus | 100.0 |
| 104692 (BA) OA Cartilage-Backus | 0.7 |
| 104694 (BA) OA Bone-Backus | 42.3 |
| 104695 (BA) Adj “Normal” Bone-Backus | 27.4 |
| 104696 (BA) OA Synovium-Backus | 71.2 |
| 104700 (SS) OA Bone-Backus | 16.4 |
| 104701 (SS) Adj “Normal” Bone-Backus | 29.7 |
| 104702 (SS) OA Synovium-Backus | 67.8 |
| 117093 OA Cartilage Rep7 | 1.8 |
| 112672 OA Bone5 | 8.8 |
| 112673 OA Synovium5 | 3.5 |
| 112674 OA Synovial Fluid cells5 | 3.7 |
| 117100 OA Cartilage Rep14 | 1.8 |
| 112756 OA Bone9 | 2.5 |
| 112757 OA Synovium9 | 4.7 |
| 112758 OA Synovial Fluid Cells9 | 9.0 |
| 117125 RA Cartilage Rep2 | 4.6 |
| 113492 Bone2 RA | 6.6 |
| 113493 Synovium2 RA | 4.4 |
| 113494 Syn Fluid Cells RA | 5.8 |
| 113499 Cartilage4 RA | 8.3 |
| 113500 Bone4 RA | 7.9 |
| 113501 Synovium4 RA | 6.9 |
| 113502 Syn Fluid Cells4 RA | 4.1 |
| 113495 Cartilage3 RA | 5.6 |
| 113496 Bone3 RA | 5.8 |
| 113497 Synovium3 RA | 4.3 |
| 113498 Syn Fluid Cells3 RA | 9.2 |
| 117106 Normal Cartilage Rep20 | 2.0 |
| 113663 Bone3 Normal | 0.0 |
| 113664 Synovium3 Normal | 0.0 |
| 113665 Syn Fluid Cells3 Normal | 0.1 |
| 117107 Normal Cartilage Rep22 | 1.4 |
| 113667 Bone4 Normal | 2.5 |
| 113668 Synovium4 Normal | 1.4 |
| 113669 Syn Fluid Cells4 Normal | 3.1 |

Column A - Rel. Exp. (%) Ag274, Run 228157466

TABLE QC
General_screenmg_panel_v1.4

| Tissue Name | A | B |
|---|---|---|
| Adipose | 20.4 | 23.5 |
| Melanoma* Hs688(A).T | 0.0 | 0.0 |
| Melanoma* Hs688(B).T | 0.6 | 1.5 |
| Melanoma* M14 | 0.0 | 0.0 |
| Melanoma* LOXIMVI | 3.1 | 0.0 |
| Melanoma* SK-MEL-5 | 0.0 | 1.9 |
| Squamous cell carcinoma SCC-4 | 1.0 | 0.0 |
| Testis Pool | 5.7 | 7.1 |
| Prostate ca.* (bone met) PC-3 | 0.0 | 1.1 |
| Prostate Pool | 1.6 | 2.0 |
| Placenta | 45.4 | 51.4 |
| Uterus Pool | 2.2 | 1.2 |
| Ovarian ca. OVCAR-3 | 0.0 | 0.0 |
| Ovarian ca. SK-OV-3 | 0.0 | 0.8 |
| Ovarian ca. OVCAR-4 | 0.0 | 0.0 |
| Ovarian ca. OVCAR-5 | 0.0 | 2.6 |
| Ovarian ca. IGROV-1 | 0.5 | 1.0 |
| Ovarian ca. OVCAR-8 | 0.8 | 2.9 |
| Ovary | 12.8 | 9.9 |
| Breast ca. MCF-7 | 0.7 | 0.0 |
| Breast ca. MDA-MB-231 | 0.4 | 2.4 |
| Breast ca. BT 549 | 0.0 | 0.0 |
| Breast ca. T47D | 1.0 | 1.6 |
| Breast ca. MDA-N | 0.6 | 1.3 |
| Breast Pool | 8.7 | 6.1 |
| Trachea | 14.3 | 21.3 |
| Lung | 3.8 | 5.3 |
| Fetal Lung | 6.7 | 4.6 |
| Lung ca. NCI-N417 | 0.0 | 0.0 |
| Lung ca. LX-1 | 0.3 | 0.6 |
| Lung ca. NCI-H146 | 1.0 | 0.0 |
| Lung ca. SHP-77 | 0.8 | 1.0 |
| Lung ca. A549 | 1.1 | 0.0 |
| Lung ca. NCI-H526 | 0.0 | 0.0 |
| Lung ca. NCI-H23 | 0.0 | 0.0 |
| Lung ca. NCI-H460 | 0.0 | 1.1 |
| Lung ca. HOP-62 | 0.0 | 5.1 |
| Lung ca. NCI-H522 | 0.0 | 0.0 |
| Liver | 11.7 | 13.5 |
| Fetal Liver | 26.6 | 26.2 |
| Liver ca. HepG2 | 0.0 | 0.7 |
| Kidney Pool | 7.0 | 13.0 |
| Fetal Kidney | 3.1 | 3.2 |
| Renal ca. 786-0 | 0.0 | 0.0 |
| Renal ca. A498 | 0.0 | 0.0 |
| Renal ca. ACHN | 0.0 | 1.8 |
| Renal ca. UO-31 | 0.0 | 0.0 |
| Renal ca. TK-10 | 0.0 | 0.6 |
| Bladder | 100.0 | 100.0 |
| Gastric ca. (liver met.) NCI-N87 | 2.0 | 2.1 |
| Gastric ca. KATO III | 0.0 | 2.0 |
| Colon ca. SW-948 | 0.0 | 0.0 |
| Colon ca. SW480 | 1.2 | 0.3 |
| Colon ca.* (SW480 met) SW620 | 0.5 | 0.5 |
| Colon ca. HT29 | 1.3 | 0.0 |
| Colon ca. HCT-116 | 0.0 | 0.8 |
| Colon ca. CaCo-2 | 1.0 | 0.0 |
| Colon cancer tissue | 26.4 | 32.3 |
| Colon ca. SW1116 | 1.6 | 0.8 |
| Colon ca. Colo-205 | 0.0 | 0.0 |
| Colon ca. SW-48 | 0.0 | 1.3 |
| Colon Pool | 12.1 | 9.7 |
| Small Intestine Pool | 5.3 | 3.8 |
| Stomach Pool | 8.1 | 9.8 |
| Bone Marrow Pool | 4.8 | 7.9 |
| Fetal Heart | 0.4 | 1.2 |
| Heart Pool | 5.3 | 8.2 |
| Lymph Node Pool | 8.8 | 11.7 |
| Fetal Skeletal Muscle | 5.0 | 5.5 |
| Skeletal Muscle Pool | 12.2 | 13.0 |
| Spleen Pool | 29.7 | 28.1 |
| Thymus Pool | 12.6 | 10.9 |
| CMS cancer (glio/astro) U87-MG | 0.9 | 1.2 |
| CMS cancer (glio/astro) U-118-MG | 1.7 | 0.8 |
| CMS cancer (neuro; met) SK-N-AS | 0.5 | 0.0 |
| CMS cancer (astro) SF-539 | 0.0 | 0.5 |
| CMS cancer (astro) SNB-75 | 1.3 | 1.1 |
| CMS cancer (glio) SNB-19 | 0.2 | 0.0 |
| CNS cancer (glio) SF-295 | 0.1 | 0.5 |
| Brain (Amygdala) Pool | 2.6 | 2.5 |
| Brain (cerebellum) | 0.8 | 1.5 |
| Brain (fetal) | 2.3 | 3.3 |
| Brain (Hippocampus) Pool | 1.0 | 2.3 |
| Cerebral Cortex Pool | 1.2 | 0.6 |
| Brain (Substantia nigra) Pool | 1.3 | 0.0 |
| Brain (Thalamus) Pool | 1.5 | 1.5 |
| Brain (whole) | 5.7 | 4.7 |
| Spinal Cord Pool | 1.1 | 2.2 |
| Adrenal Gland | 19.1 | 29.1 |
| Pituitary gland Pool | 0.5 | 4.1 |
| Salivary Gland | 3.8 | 4.5 |
| Thyroid (female) | 6.3 | 5.4 |
| Pancreatic ca. CAPAN2 | 0.0 | 0.0 |
| Pancreas Pool | 9.3 | 9.3 |

Column A - Rel. Exp. (%) Ag274, Run 216607761
Column B - Rel. Exp. (%) Ag274, Run 220361695

TABLE QD
Panel 2D

| Tissue Name | A |
|---|---|
| Normal Colon | 23.3 |
| CC Well to Mod Diff (ODO3866) | 13.2 |
| CC Margin (ODO3866) | 10.0 |
| CC Gr.2 rectosigmoid (ODO3868) | 6.0 |
| CC Margin (ODO3868) | 6.0 |
| CC Mod Diff (ODO3920) | 3.1 |
| CC Margin (ODO3920) | 10.9 |
| CC Gr.2 ascend colon (ODO3921) | 10.2 |
| CC Margin (ODO3921) | 25.5 |
| CC from Partial Hepatectomy (ODO4309) Mets | 9.7 |
| Liver Margin (ODO4309) | 48.3 |
| Colon mets to lung (OD04451-01) | 14.4 |
| Lung Margin (OD04451-02) | 17.8 |
| Normal Prostate 6546-1 | 11.0 |
| Prostate Cancer (OD04410) | 9.7 |
| Prostate Margin (OD04410) | 4.1 |
| Prostate Cancer (OD04720-01) | 2.0 |
| Prostate Margin (OD04720-02) | 4.5 |
| Normal Lung 061010 | 66.0 |
| Lung Met to Muscle (ODO4286) | 100.0 |
| Muscle Margin (ODO4286) | 72.2 |
| Lung Malignant Cancer (OD03126) | 35.4 |
| Lung Margin (OD03126) | 68.3 |
| Lung Cancer (OD04404) | 29.5 |
| Lung Margin (OD04404) | 35.6 |
| Lung Cancer (OD04565) | 12.4 |
| Lung Margin (OD04565) | 36.1 |
| Lung Cancer (OD04237-01) | 18.6 |
| Lung Margin (OD04237-02) | 22.4 |
| Ocular Mel Met to Liver (ODO4310) | 4.2 |
| Liver Margin (ODO4310) | 24.3 |
| Melanoma Mets to Lung (OD04321) | 5.8 |
| Lung Margin (OD04321) | 29.7 |
| Normal Kidney | 12.2 |
| Kidney Ca, Nuclear grade 2 (OD04338) | 16.5 |
| Kidney Margin (OD04338) | 6.2 |
| Kidney Ca Nuclear grade 1/2 (OD04339) | 23.2 |
| Kidney Margin (OD04339) | 4.6 |
| Kidney Ca, Clear cell type (OD04340) | 11.8 |
| Kidney Margin (OD04340) | 5.8 |
| Kidney Ca, Nuclear grade 3 (OD04348) | 56.6 |
| Kidney Margin (OD04348) | 22.8 |
| Kidney Cancer (OD04622-01) | 24.0 |
| Kidney Margin (OD04622-03) | 2.6 |
| Kidney Cancer (OD04450-01) | 0.9 |
| Kidney Margin (OD04450-03) | 2.6 |
| Kidney Cancer 8120607 | 5.7 |
| Kidney Margin 8120608 | 3.6 |
| Kidney Cancer 8120613 | 5.7 |
| Kidney Margin 8120614 | 3.5 |
| Kidney Cancer 9010320 | 61.1 |
| Kidney Margin 9010321 | 24.1 |
| Normal Uterus | 5.4 |
| Uterus Cancer 064011 | 5.1 |
| Normal Thyroid | 5.9 |
| Thyroid Cancer 064010 | 3.1 |
| Thyroid Cancer A302152 | 3.3 |
| Thyroid Margin A302153 | 13.5 |
| Normal Breast | 13.2 |
| Breast Cancer (OD04566) | 40.9 |
| Breast Cancer (OD04590-01) | 29.5 |
| Breast Cancer Mets (OD04590-03) | 26.1 |
| Breast Cancer Metastasis (OD04655-05) | 31.9 |
| Breast Cancer 064006 | 16.0 |
| Breast Cancer 1024 | 11.3 |
| Breast Cancer 9100266 | 3.7 |
| Breast Margin 9100265 | 4.7 |
| Breast Cancer A209073 | 5.7 |
| Breast Margin A209073 | 1.5 |
| Normal Liver | 8.4 |
| Liver Cancer 064003 | 8.4 |
| Liver Cancer 1025 | 25.3 |
| Liver Cancer 1026 | 6.4 |
| Liver Cancer 6004-T | 40.1 |
| Liver Tissue 6004-N | 17.8 |
| Liver Cancer 6005-T | 10.2 |
| Liver Tissue 6005-N | 18.6 |
| Normal Bladder | 60.7 |
| Bladder Cancer 1023 | 5.1 |
| Bladder Cancer A302173 | 18.6 |
| Bladder Cancer (OD04718-01) | 28.5 |
| Bladder Normal Adjacent (OD04718-03) | 48.6 |
| Normal Ovary | 0.0 |
| Ovarian Cancer 064008 | 16.2 |
| Ovarian Cancer (OD04768-07) | 16.4 |
| Ovary Margin (OD04768-08) | 19.2 |
| Normal Stomach | 6.4 |
| Gastric Cancer 9060358 | 2.0 |
| Stomach Margin 9060359 | 5.1 |
| Gastric Cancer 9060395 | 8.4 |
| Stomach Margin 9060394 | 10.2 |
| Gastric Cancer 9060397 | 17.4 |
| Stomach Margin 9060396 | 5.3 |
| Gastric Cancer 064005 | 14.7 |

Column A - Rel. Exp. (%) Ag274, Run 170849611

TABLE QE
Panel 3D

| Tissue Name | A |
|---|---|
| Daoy- Medulloblastoma | 14.7 |
| TE671- Medulloblastoma | 51.1 |
| D283 Med- Medulloblastoma | 0.0 |
| PFSK-1- Primitive Neuroectodermal | 0.0 |
| XF-498- CNS | 0.0 |
| SNB-78- Glioma | 10.5 |
| SF-268- Glioblastoma | 0.0 |
| T98G- Glioblastoma | 10.2 |
| SK-N-SH- Neuroblastoma (metastasis) | 10.4 |
| SF-295- Glioblastoma | 0.0 |
| Cerebellum | 22.7 |
| Cerebellum | 9.0 |
| NCI-H292- Mucoepidermoid lung carcinoma | 0.0 |
| DMS-114- Small cell lung cancer | 6.4 |
| DMS-79- Small cell lung cancer | 32.8 |
| NCI-H146- Small cell lung cancer | 0.0 |
| NCI-H526- Small cell lung cancer | 11.3 |
| NCI-N417- Small cell lung cancer | 0.0 |
| NCI-H82- Small cell lung cancer | 5.0 |
| NCI-H157- Squamous cell lung cancer (metastasis) | 0.0 |
| NCI-H1155- Large cell lung cancer | 25.2 |
| NCI-H1299- Large cell lung cancer | 0.0 |
| NCI-H727- Lung carcinoid | 0.0 |
| NCI-UMC-11- Lung carcinoid | 11.5 |
| LX-1- Small cell lung cancer | 49.7 |
| Colo-205- Colon cancer | 0.0 |
| KM12- Colon cancer | 22.7 |
| KM20L2- Colon cancer | 0.0 |
| NCI-H716- Colon cancer | 0.0 |
| SW-48- Colon adenocarcinoma | 9.7 |
| SW1116- Colon adenocarcinoma | 6.1 |
| LS 174T- Colon adenocarcinoma | 0.0 |
| SW-948- Colon adenocarcinoma | 7.5 |
| SW-480- Colon adenocarcinoma | 0.0 |
| NCI-SNU-5- Gastric carcinoma | 0.0 |
| KATO III- Gastric carcinoma | 66.0 |
| NCI-SNU-16- Gastric carcinoma | 0.0 |
| NCI-SNU-1- Gastric carcinoma | 6.3 |
| RF-1- Gastric adenocarcinoma | 5.5 |
| RF-48- Gastric adenocarcinoma | 10.4 |
| MKN-45- Gastric carcinoma | 0.0 |
| NCI-N87- Gastric carcinoma | 11.1 |
| OVCAR-5- Ovarian carcinoma | 8.8 |
| RL95-2- Uterine carcinoma | 0.0 |
| HelaS3- Cervical adenocarcinoma | 3.4 |
| Ca Ski- Cervical epidermoid carcinoma (metastasis) | 4.2 |
| ES-2- Ovarian clear cell carcinoma | 9.7 |
| Ramos- Stimulated with PMA/ionomycin 6 h | 5.6 |
| Ramos- Stimulated with PMA/ionomycin 14 h | 100.0 |
| MEG-01- Chronic myelogenous leukemia | 11.3 |
| (megokaryoblast) | |
| Raji- Burkitt's lymphoma | 5.4 |
| Daudi- Burkitt's lymphoma | 5.1 |
| U266- B-cell plasmacytoma | 7.4 |
| CA46- Burkitt's lymphoma | 0.0 |
| RL- non-Hodgkin's B-cell lymphoma | 0.0 |
| JM1- pre-B-cell lymphoma | 0.0 |
| Jurkat- T cell leukemia | 0.0 |
| TF-1- Erythroleukemia | 0.0 |
| HUT 78- T-cell lymphoma | 14.9 |
| U937- Histiocytic lymphoma | 9.1 |
| KU-812- Myelogenous leukemia | 39.5 |
| 769-P- Clear cell renal carcinoma | 6.0 |
| Caki-2- Clear cell renal carcinoma | 9.7 |
| SW 839- Clear cell renal carcinoma | 16.4 |
| Rhabdoid kidney tumor | 6.7 |
| Hs766T- Pancreatic carcinoma (LN metastasis) | 0.0 |
| CAPAN-1- Pancreatic adenocarcinoma (liver metastasis) | 11.3 |
| SU86.86- Pancreatic carcinoma (liver metastasis) | 0.0 |
| BxPC-3- Pancreatic adenocarcinoma | 0.0 |
| HPAC- Pancreatic adenocarcinoma | 0.0 |
| MIA PaCa-2- Pancreatic carcinoma | 4.5 |
| CFPAC-1- Pancreatic ductal adenocarcinoma | 7.4 |
| PANC-1 - Pancreatic epithelioid ductal carcinoma | 58.6 |
| T24- Bladder carcinma (transitional cell) | 0.0 |
| 5637- Bladder carcinoma | 11.1 |
| HT-1197- Bladder carcinoma | 0.0 |
| UM-UC-3- Bladder carcinma (transitional cell) | 10.8 |
| A204- Rhabdomyosarcoma | 0.0 |
| HT-1080- Fibrosarcoma | 0.0 |
| MG-63- Osteosarcoma | 9.9 |
| SK-LMS-1- Leiomyosarcoma (vulva) | 0.0 |
| SJRH30- Rhabdomyosarcoma (met to bone marrow) | 3.8 |
| A431- Epidermoid carcinoma | 0.0 |
| WM266-4- Melanoma | 0.0 |
| DU 145- Prostate carcinoma (brain metastasis) | 5.5 |
| MDA-MB-468- Breast adenocarcinoma | 10.8 |
| SCC-4- Squamous cell carcinoma of tongue | 0.0 |
| SCC-9- Squamous cell carcinoma of tongue | 0.0 |
| SCC-15- Squamous cell carcinoma of tongue | 0.0 |
| CAL 27- Squamous cell carcinoma of tongue | 0.0 |

Column A - Rel. Exp. (%) Ag274, Run 170745273

TABLE QF
Panel 4D

| Tissue Name | A | B |
|---|---|---|
| Secondary Th1 act | 0.0 | 0.0 |
| Secondary Th2 act | 0.4 | 0.1 |
| Secondary Tr1 act | 0.2 | 0.2 |
| Secondary Th1 rest | 0.0 | 0.0 |
| Secondary Th2 rest | 0.9 | 0.0 |
| Secondary Tr1 rest | 0.3 | 0.0 |
| Primary Th1 act | 0.4 | 0.0 |
| Primary Th2 act | 0.3 | 0.3 |
| Primary Tr1 act | 0.0 | 0.0 |
| Primary Th1 rest | 0.4 | 0.5 |
| Primary Th2 rest | 0.6 | 0.1 |
| Primary Tr1 rest | 0.0 | 0.3 |
| CD45RA CD4 lymphocyte act | 0.3 | 0.2 |
| CD45RO CD4 lymphocyte act | 0.0 | 0.0 |
| CD8 lymphocyte act | 0.0 | 0.0 |
| Secondary CD8 lymphocyte rest | 0.7 | 0.0 |
| Secondary CD8 lymphocyte act | 0.0 | 0.0 |
| CD4 lymphocyte none | 0.0 | 0.8 |
| 2ry Th1/Th2/Tr1_anti-CD95 CH11 | 0.3 | 0.0 |
| LAK cells rest | 13.3 | 12.6 |
| LAK cells IL-2 | 0.5 | 0.4 |
| LAK cells IL-2 + IL-12 | 0.5 | 1.6 |
| LAK cells IL-2 + IFN gamma | 0.8 | 0.4 |
| LAK cells IL-2 + IL-18 | 0.5 | 0.4 |
| LAK cells PMA/ionomycin | 5.4 | 6.9 |
| NK Cells IL-2 rest | 0.5 | 0.1 |
| Two Way MLR 3 day | 44.4 | 38.7 |
| Two Way MLR 5 day | 8.0 | 6.9 |
| Two Way MLR 7 day | 0.4 | 0.5 |
| PBMC rest | 1.4 | 1.7 |
| PBMC PWM | 0.7 | 0.5 |
| PBMC PHA-L | 2.6 | 3.2 |
| Ramos (B cell) none | 0.0 | 0.3 |
| Ramos (B cell) ionomycin | 0.0 | 0.7 |
| B lymphocytes PWM | 0.0 | 0.0 |
| B lymphocytes CD40L and IL-4 | 0.3 | 0.6 |
| EOL-1 dbcAMP | 0.2 | 0.4 |
| EOL-1 dbcAMP PMA/ionomycin | 0.0 | 0.3 |
| Dendritic cells none | 7.9 | 6.4 |
| Dendritic cells LPS | 32.1 | 31.6 |
| Dendritic cells anti-CD40 | 3.8 | 3.6 |
| Monocytes rest | 7.9 | 8.4 |
| Monocytes LPS | 100.0 | 100.0 |
| Macrophages rest | 3.6 | 3.5 |
| Macrophages LPS | 95.3 | 88.3 |
| HUVEC none | 0.2 | 0.0 |
| HUVEC starved | 0.4 | 0.3 |
| HUVEC IL-1beta | 0.5 | 0.0 |
| HUVEC IFN gamma | 0.0 | 0.1 |
| HUVEC TNF alpha + IFN gamma | 0.3 | 0.1 |
| HUVEC TNF alpha + IL4 | 0.0 | 0.2 |
| HUVEC IL-11 | 0.2 | 0.0 |
| Lung Microvascular EC none | 0.0 | 0.3 |
| Lung Microvascular EC TNFalpha + IL-1beta | 0.0 | 0.3 |
| Microvascular Dermal EC none | 0.0 | 0.0 |
| Microsvasular Dermal EC TNFalpha + IL-1beta | 0.0 | 0.2 |
| Bronchial epithelium TNFalpha + IL1beta | 0.0 | 0.3 |
| Small airway epithelium none | 0.0 | 0.0 |
| Small airway epithelium TNFalpha + IL-1beta | 0.0 | 0.1 |
| Coronery artery SMC rest | 0.2 | 0.0 |
| Coronery artery SMC TNFalpha + IL-1beta | 0.0 | 0.0 |
| Astrocytes rest | 0.0 | 0.0 |
| Astrocytes TNFalpha + IL-1beta | 1.0 | 0.3 |
| KU-812 (Basophil) rest | 0.0 | 0.0 |
| KU-812 (Basophil) PMA/ionomycin | 0.4 | 0.1 |
| CCD1106 (Keratinocytes) none | 0.4 | 0.0 |
| CCD1106 (Keratinocytes) TNFalpha + IL-1beta | 0.3 | 0.9 |
| Liver cirrhosis | 2.3 | 1.5 |
| Lupus kidney | 1.8 | 0.8 |
| NCI-H292 none | 0.0 | 0.4 |
| NCI-H292 IL-4 | 0.3 | 0.2 |
| NCI-H292 IL-9 | 0.0 | 0.0 |
| NCI-H292 IL-13 | 0.0 | 0.2 |
| NCI-H292 IFN gamma | 0.0 | 0.0 |
| HPAEC none | 0.3 | 0.0 |
| HPAEC TNF alpha + IL-1 beta | 0.0 | 0.0 |
| Lung fibroblast none | 0.3 | 0.0 |
| Lung fibroblast TNF alpha + IL-1 beta | 0.3 | 0.0 |
| Lung fibroblast IL-4 | 0.4 | 0.4 |
| Lung fibroblast IL-9 | 0.0 | 0.0 |
| Lung fibroblast IL-13 | 0.0 | 0.0 |
| Lung fibroblast IFN gamma | 0.0 | 0.3 |
| Dermal fibroblast CCD1070 rest | 0.7 | 0.2 |
| Dermal fibroblast CCD1070 TNF alpha | 0.4 | 0.0 |
| Dermal fibroblast CCD1070 IL-1 beta | 0.0 | 0.1 |
| Dermal fibroblast IFN gamma | 0.0 | 0.0 |
| Dermal fibroblast IL-4 | 0.4 | 0.2 |
| IBD Colitis 2 | 0.0 | 0.2 |
| IBD Crohn's | 0.6 | 0.7 |
| Colon | 4.1 | 4.1 |
| Lung | 7.5 | 8.0 |
| Thymus | 0.9 | 0.1 |
| Kidney | 4.8 | 3.0 |

Column A - Rel. Exp. (%) Ag274, Run 164533655
Column B - Rel. Exp. (%) Ag274, Run 164534874

TABLE QG
Panel 5D

| Tissue Name | A |
|---|---|
| 97457_Patient-02go_adipose | 100.0 |
| 97476_Patient-07sk_skeletal muscle | 18.9 |
| 97477_Patient-07ut_uterus | 15.2 |
| 97478_Patient-07pl_placenta | 28.9 |
| 97481_Patient-08sk_skeletal muscle | 17.1 |
| 97482_Patient-08ut_uterus | 7.4 |
| 97483_Patient-08pl_placenta | 25.9 |
| 97486_Patient-09sk_skeletal muscle | 22.1 |
| 97487_Patient-09ut_uterus | 13.7 |
| 97488_Patient-09pl_placenta | 6.7 |
| 97492_Patient-10ut_uterus | 23.0 |
| 97493_Patient-10pl_placenta | 16.5 |
| 97495_Patient-11go_adipose | 26.1 |
| 97496_Patient-11sk_skeletal muscle | 11.1 |
| 97497_Patient-11ut_uterus | 9.4 |
| 97498_Patient-11pl_placenta | 14.7 |
| 97500_Patient-12go_adipose | 25.0 |
| 97501_Patient-12sk_skeletal muscle | 7.0 |
| 97502_Patient-12ut_uterus | 37.9 |
| 97503_Patient-12pl_placenta | 9.5 |
| 94721_Donor 2 U - A_Mesenchymal Stem Cells | 6.5 |
| 94722_Donor 2 U - B_Mesenchymal Stem Cells | 0.0 |
| 94723_Donor 2 U - C_Mesenchymal Stem Cells | 0.0 |
| 94709_Donor 2 AM - A_adipose | 0.0 |
| 94710_Donor 2 AM - B_adipose | 10.3 |
| 94711_Donor 2 AM - C_adipose | 3.6 |
| 94712_Donor 2 AD - A_adipose | 0.0 |
| 94713_Donor 2 AD - B_adipose | 3.4 |
| 94714_Donor 2 AD - C_adipose | 0.0 |
| 94742_Donor 3 U - A_Mesenchymal Stem Cells | 6.3 |
| 94743_Donor 3 U - B_Mesenchymal Stem Cells | 7.4 |
| 94730_Donor 3 AM - A_adipose | 3.0 |
| 94731_Donor 3 AM - B_adipose | 10.2 |
| 94732_Donor 3 AM - C_adipose | 6.9 |
| 94733_Donor 3 AD - A_adipose | 1.6 |
| 94734_Donor 3 AD - B_adipose | 9.0 |
| 94735_Donor 3 AD - C_adipose | 6.6 |
| 77138_Liver_HepG2untreated | 7.4 |
| 73556_Heart_Cardiac stromal cells (primary) | 0.0 |
| 81735_Small Intestine | 22.1 |
| 72409_Kidney_Proximal Convoluted Tubule | 3.2 |
| 82685_Small intestine_Duodenum | 11.4 |
| 90650_Adrenal_Adrenocortical adenoma | 15.4 |
| 72410_Kidney_HRCE | 10.2 |
| 72411_Kidney_HRE | 2.9 |
| 73139_Uterus_Uterine smooth muscle cells | 3.2 |

Column A - Rel. Exp. (%) Ag274, Run 255179442

AI_comprehensive panel_v1.0 Summary: Ag274 Highest expression of this gene was detected in synovium of an osteooarthritis (OA) patient (CT=28.6). This gene showed a higher level of expression in the bone and synovium samples from OA patients relative to samples from rheumatoid arthritis patients and normal tissues. This gene encodes sialoadhesin (CD169), a lectin that binds to alpha-2,6-linked, sialic acid residues. It has been previously reported that expression of sialoadhesin is highly induced in synovial macrophages from patients with rheumatoid arthritis and in animal models of arthritis (Hartnell, A. Blood 97: 288-296; Dijkstra, C. D. Scand. J. Immunol. 26:5132). The expression profile in this panel confirmed expression of this gene in rheumatoid tissue and also demonstrated an upregulation in synovial tissue from OA patients. Modulation of this gene, expressed protein and/or use of antibodies or small molecule drug targeting the gene or gene product is useful in the treatment of osteoarthritis.

Low expression of this gene was also seen in samples from COPD lung, emphysema, atopic asthma, asthma, Crohn's disease (normal matched control and diseased), ulcerative colitis(normal matched control and diseased), and psoriasis (normal matched control and diseased). Therapeutic modulation of this gene or gene product is useful for the amelioration of symptoms/conditions associated with autoimmune and inflammatory disorders including psoriasis, asthma, inflammatory bowel disease, and rheumatoid arthritis.

General_screening₁₃ panel_v1.4 Summary: Ag274 Highest expression of this gene was detected in bladder (CTs=27-29). Expression of this gene was mainly associated with normal tissues in this panel. Among tissues with metabolic or endocrine function, this gene was expressed at moderate levels in pancreas, adipose, adrenal gland, thyroid, pituitary gland, skeletal muscle, heart, liver and the gastrointestinal tract. Therapeutic modulation of this gene or expressed protein is useful in the treatment of endocrine/metabolically related diseases, such as obesity and diabetes.

This gene was expressed at low levels in all regions of the central nervous system examined, including amygdala, hippocampus, substantia nigra, thalamus, cerebellum, cerebral cortex, and spinal cord. Therapeutic modulation of this gene product is useful in the treatment of central nervous system disorders such as Alzheimer's disease, Parkinson's disease, epilepsy, multiple sclerosis, schizophrenia and depression.

Significant expression of this gene was also seen in a colon cancer cell line. Modulation of this gene or encoded protein is useful in the treatment of colon cancer.

Panel 2D Summary: Ag274 Highest expression of this gene was detected in muscle cancer metastasized from lung (CT=29). Moderate expression of this gene was also seen in normal and cancer samples from colon, lung, kidney, liver, prostate, bladder, ovary and stomach. Therapeutic modulation of this gene or expressed protein is useful in the treatment of these cancers.

Panel 3D Summary: Ag274 Expression of this gene was highest in the Ramos cell line stimulated with PMA and ionomycin for 16 hrs, followed by lower expression in cell lines derived from gastric carcinoma and pancreatic ductal carcinoma, and lower still in small cell lung carcinoma, medulloblastoma and myelogenous leukemia.

Panel 4D Summary: Ag274 Highest expression of this gene was detected in LPS treated monocytes (CTs=27). The expression of this gene was limited to LPS activated monocytes, macrophages and related cell types or derived from monocytes (dendritic cells). The putative sialoadhesin encoded by this gene directs monocyte extravasation into tissues, and acts as a cell:cell interaction molecule. Modulation of this gene, expressed protein and/or use of antibodies is useful for blocking monocyte extravasation and reducing or inhibiting inflammation associated with asthma, psoriasis, emphysema, arthritis, and other inflammatory diseases.

Panel 5D Summary: Ag274 Highest expression of this gene was detected in adipose of a diabetic patient (CT=31). Significant expression of this gene was also seen in skeletal muscle and placenta from obese and/or diabetic patients. Therapeutic modulation of this gene or expressed protein is useful in the treatment of metabolic/endocrine diseases such as obesity and diabetes.

R. CG51992-05: CTCL Tumor Antigens E57-1

Expression of gene CG51992-05 was assessed using the primer-probe sets Ag1920, Ag2775, Ag2776, and Ag5232, described in Tables RA, RB, RC, and RD. Results of the RTQ-PCR runs are shown in Tables RE, RF, RG, and RH.

TABLE RA
Probe Name Ag1920

| | | | | |
|---|---|---|---|---|
| Primers | Sequences | Length | Start Position | SEQ ID No |

| | | | | |
|---|---|---|---|---|
| Forward | 5′-gctcctggagaatggaaatc-3′ | 20 | 902 | 1324 |
| Probe | TET-5′-tccctctgaagatcctggcaactttt-3′-TAMRA | 26 | 876 | 1325 |
| Reverse | 5′-caagctactgcaactcaaatcc-3′ | 22 | 839 | 1326 |

TABLE RB
Probe Name Ag2775

| | | | | |
|---|---|---|---|---|
| Primers | Sequences | Length | Start Position | SEQ ID No |

| | | | | |
|---|---|---|---|---|
| Forward | 5′-caggatcttcagagggagattt-3′ | 22 | 885 | 1327 |
| Probe | TET-5′-ctccaggagcagatctctcatctgca-3′-TAMRA | 26 | 912 | 1328 |
| Reverse | 5′-atgttgggagtgaatcacaaac-3′ | 22 | 938 | 1329 |

TABLE RC
Probe Name Ag2776

| | | | | |
|---|---|---|---|---|
| Primers | Sequences | Length | Start Position | SEQ ID No |

| | | | | |
|---|---|---|---|---|
| Forward | 5′-aaatgtcatcttcttgcaaagc-3′ | 22 | 1333 | 1330 |
| Probe | TET-5′-tccatttaaataatggcattttgcca-3′-TAMRA | 26 | 1289 | 1331 |
| Reverse | 5′-cagcccaagtttcatgttaaaa-3′ | 22 | 1267 | 1332 |

TABLE RD
Probe Name Ag5232

| | | | | |
|---|---|---|---|---|
| Primers | Sequences | Length | Start Position | SEQ ID No |

| | | | | |
|---|---|---|---|---|
| Forward | 5′-ctagatgcaaagctgcaagc-3′ | 20 | 531 | 1333 |
| Probe | TET-5′-aagcaggagcagcagctctgagaaac-3′-TAMRA | 26 | 559 | 1334 |
| Reverse | 5′-cttcttcagattgcgtttgg-3′ | 20 | 611 | 1335 |

TABLE RE
General_screening_panel_v1.5

| Tissue Name | A |
|---|---|
| Adipose | 3.7 |
| Melanoma* Hs688(A).T | 0.0 |
| Melanoma* Hs688(B).T | 1.6 |
| Melanoma* M14 | 0.2 |
| Melanoma* LOXIMVI | 0.0 |
| Melanoma* SK-MEL-5 | 0.0 |
| Squamous cell carcinoma SCC-4 | 3.1 |
| Testis Pool | 7.9 |
| Prostate ca.* (bone met) PC-3 | 3.9 |
| Prostate Pool | 4.1 |
| Placenta | 1.4 |
| Uterus Pool | 1.3 |
| Ovarian ca. OVCAR-3 | 1.5 |
| Ovarian ca. SK-OV-3 | 28.9 |
| Ovarian ca. OVCAR-4 | 0.3 |
| Ovarian ca. OVCAR-5 | 2.9 |
| Ovarian ca. IGROV-1 | 0.0 |
| Ovarian ca. OVCAR-8 | 0.1 |
| Ovary | 9.4 |
| Breast ca. MCF-7 | 0.1 |
| Breast ca. MDA-MB-231 | 11.4 |
| Breast ca. BT 549 | 71.7 |
| Breast ca. T47D | 0.0 |
| Breast ca. MDA-N | 0.0 |
| Breast Pool | 4.0 |
| Trachea | 3.2 |
| Lung | 5.1 |
| Fetal Lung | 100.0 |
| Lung ca. NCI-N417 | 0.0 |
| Lung ca. LX-1 | 2.2 |
| Lung ca. NCI-H146 | 0.0 |
| Lung ca. SHP-77 | 0.0 |
| Lung ca. A549 | 11.3 |
| Lung ca. NCI-H526 | 0.0 |
| Lung ca. NCI-H23 | 1.5 |
| Lung ca. NCI-H460 | 94.0 |
| Lung ca. HOP-62 | 1.2 |
| Lung ca. NCI-H522 | 0.0 |
| Liver | 0.0 |
| Fetal Liver | 20.9 |
| Liver ca. HepG2 | 19.8 |
| Kidney Pool | 6.0 |
| Fetal Kidney | 12.9 |
| Renal ca. 786-0 | 3.0 |
| Renal ca. A498 | 1.6 |
| Renal ca. ACHN | 1.0 |
| Renal ca. UO-31 | 8.4 |
| Renal ca. TK-10 | 7.6 |
| Bladder | 38.4 |
| Gastric ca. (liver met.) NCI-N87 | 4.4 |
| Gastric ca. KATO III | 53.2 |
| Colon ca. SW-948 | 17.9 |
| Colon ca. SW480 | 1.2 |
| Colon ca.* (SW480 met) SW620 | 0.1 |
| Colon ca. HT29 | 1.3 |
| Colon ca. HCT-116 | 7.3 |
| Colon ca. CaCo-2 | 7.5 |
| Colon cancer tissue | 7.0 |
| Colon ca. SW1116 | 0.8 |
| Colon ca. Colo-205 | 0.6 |
| Colon ca. SW-48 | 1.0 |
| Colon Pool | 1.8 |
| Small Intestine Pool | 2.5 |
| Stomach Pool | 4.1 |
| Bone Marrow Pool | 3.4 |
| Fetal Heart | 1.7 |
| Heart Pool | 7.2 |
| Lymph Node Pool | 6.2 |
| Fetal Skeletal Muscle | 0.0 |
| Skeletal Muscle Pool | 11.8 |
| Spleen Pool | 15.0 |
| Thymus Pool | 2.2 |
| CNS cancer (glio/astro) U87-MG | 0.0 |
| CNS cancer (glio/astro) U-118-MG | 0.0 |
| CNS cancer (neuro; met) SK-N-AS | 0.1 |
| CNS cancer (astro) SF-539 | 0.0 |
| CNS cancer (astro) SNB-75 | 0.3 |
| CNS cancer (glio) SNB-19 | 0.0 |
| CNS cancer (glio) SF-295 | 0.0 |
| Brain (Amygdala) Pool | 1.8 |
| Brain (cerebellum) | 1.0 |
| Brain (fetal) | 0.4 |
| Brain (Hippocampus) Pool | 0.3 |
| Cerebral Cortex Pool | 3.1 |
| Brain (Substantia nigra) Pool | 0.8 |
| Brain (Thalamus) Pool | 2.3 |
| Brain (whole) | 0.1 |
| Spinal Cord Pool | 2.1 |
| Adrenal Gland | 1.5 |
| Pituitary gland Pool | 3.2 |
| Salivary Gland | 0.4 |
| Thyroid (female) | 0.2 |
| Pancreatic ca. CAPAN2 | 1.6 |
| Pancreas Pool | 9.9 |

Column A - Rel. Exp. (%) Ag5232, Run 229385253

TABLE RF
Panel 1.3D

| Tissue Name | A | B |
|---|---|---|
| Liver adenocarcinoma | 1.9 | 3.8 |
| Pancreas | 1.2 | 0.8 |
| Pancreatic ca. CAPAN 2 | 1.4 | 0.9 |
| Adrenal gland | 4.0 | 10.7 |
| Thyroid | 3.3 | 2.6 |
| Salivary gland | 2.3 | 2.2 |
| Pituitary gland | 14.7 | 11.8 |
| Brain (fetal) | 0.2 | 0.0 |
| Brain (whole) | 10.4 | 9.7 |
| Brain (amygdala) | 5.5 | 3.7 |
| Brain (cerebellum) | 0.2 | 0.7 |
| Brain (hippocampus) | 19.5 | 30.1 |
| Brain (substantia nigra) | 2.0 | 1.8 |
| Brain (thalamus) | 8.8 | 13.4 |
| Cerebral Cortex | 20.0 | 14.3 |
| Spinal cord | 3.5 | 3.7 |
| glio/astro U87-MG | 0.5 | 0.0 |
| glio/astro U-118-MG | 0.0 | 0.0 |
| astrocytoma SW1783 | 0.2 | 0.0 |
| neuro*; met SK-N-AS | 0.6 | 0.0 |
| astrocytoma SF-539 | 0.0 | 0.0 |
| astrocytoma SNB-75 | 10.3 | 4.8 |
| glioma SNB-19 | 0.0 | 0.0 |
| glioma U251 | 0.3 | 0.0 |
| glioma SF-295 | 0.2 | 0.0 |
| Heart (fetal) | 1.7 | 1.0 |
| Heart | 8.9 | 11.2 |
| Skeletal muscle (fetal) | 2.7 | 0.7 |
| Skeletal muscle | 1.7 | 2.0 |
| Bone marrow | 2.7 | 3.9 |
| Thymus | 0.0 | 0.0 |
| Spleen | 16.3 | 9.2 |
| Lymph node | 4.3 | 4.1 |
| Colorectal | 42.0 | 12.5 |
| Stomach | 29.9 | 7.1 |
| Small intestine | 38.4 | 40.6 |
| Colon ca. SW480 | 1.2 | 0.0 |
| Colon ca.* SW620(SW480 met) | 0.0 | 0.0 |
| Colon ca. HT29 | 4.7 | 0.5 |
| Colon ca. HCT-116 | 5.3 | 3.5 |
| Colon ca. CaCo-2 | 10.7 | 4.7 |
| Colon ca. tissue(ODO3866) | 6.3 | 3.5 |
| Colon ca. HCC-2998 | 5.6 | 4.7 |
| Gastric ca.* (liver met) NCI-N87 | 7.7 | 3.3 |
| Bladder | 22.1 | 21.5 |
| Trachea | 15.3 | 4.4 |
| Kidney | 2.2 | 4.0 |
| Kidney (fetal) | 8.4 | 14.9 |
| Renal ca. 786-0 | 7.6 | 2.5 |
| Renal ca. A498 | 10.7 | 4.3 |
| Renal ca. RXF 393 | 2.1 | 0.0 |
| Renal ca. ACHN | 0.9 | 0.0 |
| Renal ca. UO-31 | 5.3 | 0.0 |
| Renal ca. TK-10 | 4.5 | 1.8 |
| Liver | 5.3 | 9.5 |
| Liver (fetal) | 21.8 | 39.0 |
| Liver ca. (hepatoblast) HepG2 | 19.5 | 9.7 |
| Lung | 38.7 | 53.6 |
| Lung (fetal) | 100.0 | 100.0 |
| Lung ca. (small cell) LX-1 | 2.8 | 2.3 |
| Lung ca. (small cell) NCI-H69 | 0.0 | 0.0 |
| Lung ca. (s. cell var.) SHP-77 | 0.0 | 0.0 |
| Lung ca. (large cell)NCI-H460 | 75.3 | 38.4 |
| Lung ca. (non-sm. cell) A549 | 12.2 | 10.0 |
| Lung ca. (non-s. cell) NCI-H23 | 2.3 | 2.3 |
| Lung ca. (non-s. cell) HOP-62 | 2.2 | 0.6 |
| Lung ca. (non-s. cl) NCI-H522 | 0.0 | 0.0 |
| Lung ca. (squam.) SW 900 | 9.3 | 12.0 |
| Lung ca. (squam.) NCI-H596 | 0.0 | 0.0 |
| Mammary gland | 3.8 | 1.9 |
| Breast ca.* (pl. ef) MCF-7 | 0.0 | 0.0 |
| Breast ca.* (pl. ef) MDA-MB-231 | 15.5 | 10.4 |
| Breast ca.* (pl. ef) T47D | 0.0 | 0.0 |
| Breast ca. BT-549 | 10.2 | 6.2 |
| Breast ca. MDA-N | 0.0 | 0.0 |
| Ovary | 13.1 | 7.7 |
| Ovarian ca. OVCAR-3 | 1.7 | 0.6 |
| Ovarian ca. OVCAR-4 | 0.1 | 0.0 |
| Ovarian ca. OVCAR-5 | 7.3 | 1.2 |
| Ovarian ca. OVCAR-8 | 2.4 | 0.0 |
| Ovarian ca. IGROV-1 | 0.0 | 0.0 |
| Ovarian ca.* (ascites) SK-OV-3 | 9.7 | 4.0 |
| Uterus | 9.6 | 6.3 |
| Placenta | 34.2 | 37.6 |
| Prostate | 12.9 | 12.6 |
| Prostate ca.* (bone met)PC-3 | 1.2 | 0.8 |
| Testis | 39.5 | 13.0 |
| Melanoma Hs688(A).T | 0.0 | 0.0 |
| Melanoma* (met) Hs688(B).T | 0.3 | 0.0 |
| Melanoma UACC-62 | 0.0 | 0.0 |
| Melanoma M14 | 0.0 | 0.0 |
| Melanoma LOX IMVI | 0.0 | 0.0 |
| Melanoma* (met) SK-MEL-5 | 0.0 | 0.0 |
| Adipose | 3.9 | 3.4 |

Column A - Rel. Exp. (%) Ag2775, Run 153780557
Column B - Rel. Exp. (%) Ag2776, Run 153792445

TABLE RG
Panel 2D

| Tissue Name | A | B |
|---|---|---|
| Normal Colon | 94.0 | 62.4 |
| CC Well to Mod Diff (ODO3866) | 5.0 | 3.1 |
| CC Margin (ODO3866) | 21.6 | 18.8 |
| CC Gr.2 rectosigmoid (ODO3868) | 4.5 | 2.0 |
| CC Margin (ODO3868) | 1.4 | 1.2 |
| CC Mod Diff (ODO3920) | 8.2 | 4.9 |
| CC Margin (ODO3920) | 34.4 | 28.5 |
| CC Gr.2 ascend colon (ODO3921) | 20.0 | 17.7 |
| CC Margin (ODO3921) | 12.2 | 10.2 |
| CC from Partial Hepatectomy | 11.3 | 4.8 |
| (ODO4309) Mets | | |
| Liver Margin (ODO4309) | 10.2 | 5.6 |
| Colon mets to lung (OD04451-01) | 4.7 | 0.8 |
| Lung Margin (OD04451-02) | 14.2 | 6.4 |
| Normal Prostate 6546-1 | 7.6 | 5.4 |
| Prostate Cancer (OD04410) | 2.9 | 2.7 |
| Prostate Margin (OD04410) | 7.0 | 3.4 |
| Prostate Cancer (OD04720-01) | 7.0 | 4.0 |
| Prostate Margin (OD04720-02) | 8.1 | 6.5 |
| Normal Lung 061010 | 44.1 | 45.4 |
| Lung Met to Muscle (ODO4286) | 4.3 | 5.2 |
| Muscle Margin (ODO4286) | 1.5 | 0.6 |
| Lung Malignant Cancer (OD03126) | 12.3 | 5.6 |
| Lung Margin (OD03126) | 33.7 | 13.8 |
| Lung Cancer (OD04404) | 4.1 | 0.9 |
| Lung Margin (OD04404) | 11.5 | 3.0 |
| Lung Cancer (OD04565) | 2.9 | 0.6 |
| Lung Margin (OD04565) | 19.6 | 3.7 |
| Lung Cancer (OD04237-01) | 6.8 | 3.4 |
| Lung Margin (OD04237-02) | 22.4 | 9.3 |
| Ocular Mel Met to Liver (ODO4310) | 11.3 | 5.8 |
| Liver Margin (ODO4310) | 10.4 | 6.0 |
| Melanoma Mets to Lung (OD04321) | 0.3 | 0.0 |
| Lung Margin (OD04321) | 27.9 | 31.6 |
| Normal Kidney | 7.0 | 3.6 |
| Kidney Ca, Nuclear grade 2 (OD04338) | 1.7 | 0.4 |
| Kidney Margin (OD04338) | 6.0 | 0.5 |
| Kidney Ca Nuclear grade 1/2 (OD04339) | 4.7 | 0.4 |
| Kidney Margin (OD04339) | 11.4 | 1.8 |
| Kidney Ca, Clear cell type (OD04340) | 0.9 | 0.0 |
| Kidney Margin (OD04340) | 10.6 | 3.5 |
| Kidney Ca, Nuclear grade 3 (OD04348) | 0.6 | 0.2 |
| Kidney Margin (OD04348) | 6.4 | 1.6 |
| Kidney Cancer (OD04622-01) | 0.0 | 0.0 |
| Kidney Margin (OD04622-03) | 0.9 | 0.2 |
| Kidney Cancer (OD04450-01) | 4.1 | 1.5 |
| Kidney Margin (OD04450-03) | 5.6 | 1.6 |
| Kidney Cancer 8120607 | 0.0 | 0.0 |
| Kidney Margin 8120608 | 0.1 | 0.0 |
| Kidney Cancer 8120613 | 1.3 | 0.2 |
| Kidney Margin 8120614 | 0.3 | 0.0 |
| Kidney Cancer 9010320 | 1.5 | 0.0 |
| Kidney Margin 9010321 | 1.0 | 0.0 |
| Normal Uterus | 1.8 | 0.8 |
| Uterus Cancer 064011 | 4.6 | 1.4 |
| Normal Thyroid | 3.6 | 1.6 |
| Thyroid Cancer 064010 | 1.7 | 1.1 |
| Thyroid Cancer A302152 | 0.1 | 0.2 |
| Thyroid Margin A302153 | 2.9 | 1.1 |
| Normal Breast | 2.8 | 1.2 |
| Breast Cancer (OD04566) | 2.9 | 0.2 |
| Breast Cancer (OD04590-01) | 1.5 | 0.5 |
| Breast Cancer Mets (OD04590-03) | 4.8 | 1.5 |
| Breast Cancer Metastasis | 0.7 | 0.4 |
| (OD04655-05) | | |
| Breast Cancer 064006 | 2.9 | 1.4 |
| Breast Cancer 1024 | 0.4 | 0.0 |
| Breast Cancer 9100266 | 0.4 | 0.0 |
| Breast Margin 9100265 | 0.1 | 0.2 |
| Breast Cancer A209073 | 1.4 | 0.8 |
| Breast Margin A209073 | 1.5 | 1.0 |
| Normal Liver | 7.7 | 1.7 |
| Liver Cancer 064003 | 8.7 | 6.6 |
| Liver Cancer 1025 | 3.3 | 0.8 |
| Liver Cancer 1026 | 0.8 | 0.3 |
| Liver Cancer 6004-T | 3.8 | 0.7 |
| Liver Tissue 6004-N | 4.7 | 0.7 |
| Liver Cancer 6005-T | 0.8 | 0.2 |
| Liver Tissue 6005-N | 0.4 | 0.3 |
| Normal Bladder | 33.7 | 27.2 |
| Bladder Cancer 1023 | 5.1 | 3.1 |
| Bladder Cancer A302173 | 1.3 | 0.6 |
| Bladder Cancer (OD04718-01) | 12.2 | 9.6 |
| Bladder Normal Adjacent (OD04718-03) | 0.6 | 0.5 |
| Normal Ovary | 2.0 | 1.5 |
| Ovarian Cancer 064008 | 5.8 | 4.0 |
| Ovarian Cancer (OD04768-07) | 0.1 | 0.0 |
| Ovary Margin (OD04768-08) | 0.2 | 0.1 |
| Normal Stomach | 25.9 | 12.3 |
| Gastric Cancer 9060358 | 1.5 | 0.9 |
| Stomach Margin 9060359 | 4.2 | 6.3 |
| Gastric Cancer 9060395 | 14.6 | 9.8 |
| Stomach Margin 9060394 | 8.7 | 12.4 |
| Gastric Cancer 9060397 | 10.9 | 13.5 |
| Stomach Margin 9060396 | 4.8 | 3.3 |
| Gastric Cancer 064005 | 100.0 | 100.0 |

Column A - Rel. Exp. (%) Ag2775, Run 153780568
Column B - Rel. Exp. (%) Ag2776, Run 153792446

TABLE RH
Panel 4D

| Tissue Name | A | B | C |
|---|---|---|---|
| Secondary Th1 act | 0.0 | 0.0 | 0.0 |
| Secondary Th2 act | 0.4 | 0.0 | 0.6 |
| Secondary Tr1 act | 0.0 | 0.0 | 0.5 |
| Secondary Th1 rest | 0.2 | 0.0 | 0.0 |
| Secondary Th2 rest | 0.0 | 0.0 | 0.2 |
| Secondary Tr1 rest | 0.0 | 0.0 | 0.0 |
| Primary Th1 act | 0.0 | 0.0 | 0.0 |
| Primary Th2 act | 0.0 | 0.0 | 0.2 |
| Primary Tr1 act | 0.2 | 0.2 | 0.0 |
| Primary Th1 rest | 0.0 | 0.1 | 0.0 |
| Primary Th2 rest | 0.0 | 0.0 | 0.2 |
| Primary Tr1 rest | 0.1 | 0.0 | 0.0 |
| CD45RA CD4 lymphocyte act | 0.3 | 0.8 | 0.3 |
| CD45RO CD4 lymphocyte act | 0.0 | 0.0 | 0.3 |
| CD8 lymphocyte act | 0.3 | 0.0 | 0.0 |
| Secondary CD8 lymphocyte rest | 0.0 | 0.0 | 0.0 |
| Secondary CD8 lymphocyte act | 0.0 | 0.0 | 0.2 |
| CD4 lymphocyte none | 0.0 | 0.2 | 0.0 |
| 2ry Th1/Th2/Tr1_anti-CD95 CH11 | 0.0 | 0.0 | 0.3 |
| LAK cells rest | 0.0 | 0.1 | 0.8 |
| LAK cells IL-2 | 0.7 | 0.0 | 0.0 |
| LAK cells IL-2 + IL-12 | 0.2 | 0.4 | 0.0 |
| LAK cells IL-2 + IFN gamma | 0.9 | 0.4 | 0.9 |
| LAK cells IL-2 + IL-18 | 1.0 | 0.0 | 0.6 |
| LAK cells PMA/ionomycin | 0.6 | 0.0 | 0.0 |
| NK Cells IL-2 rest | 0.0 | 0.0 | 0.0 |
| Two Way MLR 3 day | 0.2 | 0.4 | 0.4 |
| Two Way MLR 5 day | 0.0 | 0.0 | 0.0 |
| Two Way MLR 7 day | 0.0 | 0.0 | 0.0 |
| PBMC rest | 0.0 | 0.0 | 0.0 |
| PBMC PWM | 0.6 | 0.0 | 0.7 |
| PBMC PHA-L | 0.0 | 0.4 | 0.0 |
| Ramos (B cell) none | 0.0 | 0.0 | 0.0 |
| Ramos (B cell) ionomycin | 0.0 | 0.0 | 0.0 |
| B lymphocytes PWM | 0.6 | 0.4 | 0.6 |
| B lymphocytes CD40L and IL-4 | 0.0 | 0.0 | 0.0 |
| EOL-1 dbcAMP | 0.2 | 0.0 | 0.0 |
| EOL-1 dbcAMP PMA/ionomycin | 0.0 | 0.0 | 0.0 |
| Dendritic cells none | 0.0 | 0.0 | 0.0 |
| Dendritic cells LPS | 0.4 | 0.0 | 0.0 |
| Dendritic cells anti-CD40 | 0.0 | 0.0 | 0.0 |
| Monocytes rest | 0.0 | 0.0 | 0.0 |
| Monocytes LPS | 0.5 | 0.0 | 0.0 |
| Macrophages rest I | 0.0 | 0.4 | 0.0 |
| Macrophages LPS | 0.0 | 0.0 | 0.0 |
| HUVEC none | 10.8 | 8.4 | 5.8 |
| HUVEC starved | 20.7 | 12.9 | 7.1 |
| HUVEC IL-1 beta | 5.6 | 5.6 | 2.3 |
| HUVEC IFN gamma | 22.1 | 19.9 | 8.2 |
| HUVEC TNF alpha + IFN gamma | 8.4 | 8.8 | 9.2 |
| HUVEC TNF alpha + IL4 | 5.3 | 5.4 | 3.4 |
| HUVEC IL-11 | 2.8 | 3.1 | 2.1 |
| Lung Microvascular EC none | 25.5 | 11.7 | 15.1 |
| Lung Microvascular EC | 12.4 | 10.2 | 9.3 |
| TNFalpha + IL-1beta | | | |
| Microvascular Dermal EC none | 23.3 | 16.5 | 28.5 |
| Microsvasular Dermal EC | 12.4 | 9.4 | 9.9 |
| TNFalpha + IL-1beta | | | |
| Bronchial epithelium TNFalpha + | 18.7 | 2.9 | 2.3 |
| IL1beta | | | |
| Small airway epithelium none | 9.5 | 6.2 | 8.1 |
| Small airway epithelium | 82.4 | 67.4 | 31.2 |
| TNFalpha + IL-1 beta | | | |
| Coronery artery SMC rest | 20.0 | 10.7 | 4.6 |
| Coronery artery SMC TNFalpha + | 10.7 | 10.6 | 2.6 |
| IL-1beta | | | |
| Astrocytes rest | 0.4 | 0.7 | 0.9 |
| Astrocytes TNFalpha + IL-1beta | 2.3 | 1.8 | 1.4 |
| KU-812 (Basophil) rest | 0.9 | 0.3 | 0.4 |
| KU-812 (Basophil) PMA/ionomycin | 4.5 | 4.5 | 2.9 |
| CCD1106 (Keratinocytes) none | 9.0 | 6.6 | 7.6 |
| CCD1106 (Keratinocytes) | 4.6 | 0.8 | 2.6 |
| TNFalpha + IL-1beta | | | |
| Liver cirrhosis | 9.0 | 12.1 | 5.7 |
| Lupus kidney | 1.4 | 1.6 | 0.9 |
| NCI-H292 none | 36.3 | 29.5 | 27.9 |
| NCI-H292 IL-4 | 76.8 | 77.4 | 60.3 |
| NCI-H292 IL-9 | 72.7 | 67.4 | 39.2 |
| NCI-H292 IL-13 | 35.1 | 34.2 | 12.9 |
| NCI-H292 IFN gamma | 100.0 | 100.0 | 59.9 |
| HPAEC none | 5.3 | 3.7 | 2.7 |
| HPAEC TNF alpha + IL-1 beta | 4.8 | 4.2 | 4.3 |
| Lung fibroblast none | 13.2 | 8.7 | 4.9 |
| Lung fibroblast TNF alpha + IL-1 | 30.8 | 28.9 | 20.9 |
| beta | | | |
| Lung fibroblast IL-4 | 22.4 | 18.7 | 13.8 |
| Lung fibroblast IL-9 | 12.4 | 9.2 | 11.6 |
| Lung fibroblast IL-13 | 11.4 | 8.0 | 13.9 |
| Lung fibroblast IFN gamma | 88.9 | 78.5 | 97.3 |
| Dermal fibroblast CCD1070 rest | 2.3 | 3.3 | 2.6 |
| Dermal fibroblast CCD1070 TNF alpha | 3.6 | 3.3 | 3.2 |
| Dermal fibroblast CCD1070 IL-1 beta | 0.8 | 0.8 | 0.5 |
| Dermal fibroblast IFN gamma | 0.2 | 0.2 | 0.0 |
| Dermal fibroblast IL-4 | 0.7 | 0.0 | 0.0 |
| IBD Colitis 2 | 0.2 | 1.4 | 0.0 |
| IBD Crohn's | 9.8 | 17.6 | 10.5 |
| Colon | 83.5 | 72.2 | 100.0 |
| Lung | 40.3 | 27.4 | 44.4 |
| Thymus | 21.6 | 18.4 | 23.8 |
| Kidney | 2.2 | 2.1 | 1.7 |

Column A - Rel. Exp. (%) Ag1920, Run 158535362
Column B - Rel. Exp. (%) Ag2775, Run 153780789
Column C - Rel. Exp. (%) Ag2776, Run 153792447

General_screening₁₃ panel_v1.5 Summary: Ag5232 Highest expression was seen in fetal lung (CT=29.3), with moderate levels of expression also seen in breast, ovarian, lung and gastric cancer cell lines.

Panel 1.3D Summary: Ag2775/Ag2776 Expression of this gene was highest in fetal lung. This gene was expressed at higher levels in normal tissues than in the cancer cell lines. Significant expression of this gene was seen throughout the brain as well as in liver, small intestine, placenta, and bladder.

Panel 2D Summary: Ag2775/Ag2776 Expression of this gene was highest in a gastric cancer sample. Expression of this gene was lower in lung and kidney tumors when compared to the matched normal adjacent tissue. These results were consistent with what is observed in Panel 1.3D. Targeting of this gene or gene product through the use of protein, antibody, or small molecule therapeutics is beneficial in the treatment of lung and kidney cancer.

Panel 4D Summary: Ag1920/Ag2775/Ag2776 Expression of this gene was highest in lung cells, including in lung fibroblasts, small airway epithelium and the NCI-H292 cell line, a human airway epithelial cell line that produces mucins. These results were consistent with what is observed in Panels 1.3D and 2D. The expression of this gene in cells derived from or within the lung shows that this gene is involved in normal conditions as well as pathological and inflammatory lung disorders that include chronic obstructive pulmonary disease, asthma, allergy and emphysema.

Moderate expression of this gene was also seen in colon and thymus.

S. CG52171-04: Hematopoietic PBX-interacting Protein

Expression of gene CG52171-04 was assessed using the primer-probe set Ag2779, described in Table SA. Results of the RTQ-PCR runs are shown in Tables SB, SC, SD, SE and SF.

TABLE SA
Probe Name Ag2779

| | | | | |
|---|---|---|---|---|
| Primers | Sequences | Length | Start Position | SEQ ID No |

| | | | | |
|---|---|---|---|---|
| Forward | 5′-caacagacaggtgatgatgatg-3′ | 22 | 2097 | 1336 |
| Probe | TET-5′-tgactttgaggacttcatcttcagcca-3′-TAMRA | 27 | 2126 | 1337 |
| Reverse | 5′-cagtgctttgtctccaaagaag-3′ | 22 | 2153 | 1338 |

TABLE SB
AI_comprehensive panel_v1.0

| Tissue Name | A |
|---|---|
| 110967 COPD-F | 32.8 |
| 110980 COPD-F | 28.7 |
| 110968 COPD-M | 45.4 |
| 110977 COPD-M | 70.7 |
| 110989 Emphysema-F | 73.7 |
| 110992 Emphysema-F | 25.7 |
| 110993 Emphysema-F | 27.2 |
| 110994 Emphysema-F | 14.9 |
| 110995 Emphysema-F | 43.5 |
| 110996 Emphysema-F | 10.7 |
| 110997 Asthma-M | 8.7 |
| 111001 Asthma-F | 25.0 |
| 111002 Asthma-F | 33.9 |
| 111003 Atopic Asthma-F | 27.2 |
| 111004 Atopic Asthma-F | 43.8 |
| 111005 Atopic Asthma-F | 27.4 |
| 111006 Atopic Asthma-F | 7.3 |
| 111417 Allergy-M | 1.2 |
| 112347 Allergy-M | 2.9 |
| 112349 Normal Lung-F | 2.4 |
| 112357 Normal Lung-F | 100.0 |
| 112354 Normal Lung-M | 32.1 |
| 112374 Crohns-F | 24.1 |
| 112389 Match Control Crohns-F | 17.7 |
| 112375 Crohns-F | 25.9 |
| 112732 Match Control Crohns-F | 39.5 |
| 112725 Crohns-M | 3.0 |
| 112387 Match Control Crohns-M | 31.6 |
| 112378 Crohns-M | 2.3 |
| 112390 Match Control Crohns-M | 45.7 |
| 112726 Crohns-M | 45.4 |
| 112731 Match Control Crohns-M | 29.5 |
| 112380 Ulcer Col-F | 27.4 |
| 112734 Match Control Ulcer Col-F | 57.8 |
| 112384 Ulcer Col-F | 35.4 |
| 112737 Match Control Ulcer Col-F | 11.6 |
| 112386 Ulcer Col-F | 19.3 |
| 112738 Match Control Ulcer Col-F | 13.3 |
| 112381 Ulcer Col-M | 3.0 |
| 112735 Match Control Ulcer Col-M | 11.0 |
| 112382 Ulcer Col-M | 14.8 |
| 112394 Match Control Ulcer Col-M | 8.5 |
| 112383 Ulcer Col-M | 26.1 |
| 112736 Match Control Ulcer Col-M | 9.9 |
| 112423 Psoriasis-F | 18.3 |
| 112427 Match Control Psoriasis-F | 71.7 |
| 112418 Psoriasis-M | 28.1 |
| 112723 Match Control Psoriasis-M | 35.6 |
| 112419 Psoriasis-M | 45.1 |
| 112424 Match Control Psoriasis-M | 15.7 |
| 112420 Psoriasis-M | 62.0 |
| 112425 Match Control Psoriasis-M | 40.1 |
| 104689 (MF) OA Bone-Backus | 31.2 |
| 104690 (MF) Adj “Normal” Bone-Backus | 21.0 |
| 104691 (MF) OA Synovium-Backus | 41.5 |
| 104692 (BA) OA Cartilage-Backus | 64.2 |
| 104694 (BA) OA Bone-Backus | 28.5 |
| 104695 (BA) Adj “Normal” Bone-Backus | 35.1 |
| 104696 (BA) OA Synovium-Backus | 28.7 |
| 104700 (SS) OA Bone-Backus | 18.8 |
| 104701 (SS) Adj “Normal” Bone-Backus | 29.5 |
| 104702 (SS) OA Synovium-Backus | 62.9 |
| 117093 OA Cartilage Rep7 | 46.7 |
| 112672 OA Bone5 | 74.7 |
| 112673 OA Synovium5 | 28.9 |
| 112674 OA Synovial Fluid cells5 | 28.5 |
| 117100 OA Cartilage Rep14 | 20.7 |
| 112756 OA Bone9 | 16.3 |
| 112757 OA Synovium9 | 45.1 |
| 112758 OA Synovial Fluid Cells9 | 14.4 |
| 117125 RA Cartilage Rep2 | 77.9 |
| 113492 Bone2 RA | 24.8 |
| 113493 Synovium2 RA | 8.1 |
| 113494 Syn Fluid Cells RA | 14.3 |
| 113499 Cartilage4 RA | 18.3 |
| 113500 Bone4 RA | 22.5 |
| 113501 Synovium4 RA | 14.1 |
| 113502 Syn Fluid Cells4 RA | 10.7 |
| 113495 Cartilage3 RA | 17.0 |
| 113496 Bone3 RA | 19.8 |
| 113497 Synovium3 RA | 11.9 |
| 113498 Syn Fluid Cells3 RA | 28.1 |
| 117106 Normal Cartilage Rep20 | 50.0 |
| 113663 Bone3 Normal | 3.3 |
| 113664 Synovium3 Normal | 1.1 |
| 113665 Syn Fluid Cells3 Normal | 2.0 |
| 117107 Normal Cartilage Rep22 | 6.4 |
| 113667 Bone4 Normal | 21.6 |
| 113668 Synovium4 Normal | 24.8 |
| 113669 Syn Fluid Cells4 Normal | 48.6 |

Column A - Rel. Exp. (%) Ag2779, Run 249247310

TABLE SC
Panel 1.3D

| Tissue Name | A |
|---|---|
| Liver adenocarcinoma | 3.5 |
| Pancreas | 1.2 |
| Pancreatic ca. CAPAN 2 | 0.3 |
| Adrenal gland | 3.7 |
| Thyroid | 12.1 |
| Salivary gland | 2.1 |
| Pituitary gland | 14.9 |
| Brain (fetal) | 0.6 |
| Brain (whole) | 4.0 |
| Brain (amygdala) | 7.0 |
| Brain (cerebellum) | 2.1 |
| Brain (hippocampus) | 11.0 |
| Brain (substantia nigra) | 2.5 |
| Brain (thalamus) | 6.4 |
| Cerebral Cortex | 10.0 |
| Spinal cord | 17.2 |
| glio/astro U87-MG | 2.8 |
| glio/astro U-118-MG | 1.6 |
| astrocytoma SW1783 | 2.9 |
| neuro*; met SK-N-AS | 2.2 |
| astrocytoma SF-539 | 3.3 |
| astrocytoma SNB-75 | 2.6 |
| glioma SNB-19 | 3.2 |
| glioma U251 | 2.5 |
| glioma SF-295 | 4.6 |
| Heart (fetal) | 15.8 |
| Heart | 4.1 |
| Skeletal muscle (fetal) | 100.0 |
| Skeletal muscle | 24.8 |
| Bone marrow | 1.7 |
| Thymus | 30.8 |
| Spleen | 11.3 |
| Lymph node | 5.9 |
| Colorectal | 8.5 |
| Stomach | 4.2 |
| Small intestine | 7.7 |
| Colon ca. SW480 | 0.5 |
| Colon ca.* SW620(SW480 met) | 0.4 |
| Colon ca. HT29 | 1.5 |
| Colon ca. HCT-116 | 0.3 |
| Colon ca. CaCo-2 | 3.0 |
| Colon ca. tissue(ODO3866) | 2.8 |
| Colon ca. HCC-2998 | 0.9 |
| Gastric ca.* (liver met) NCI-N87 | 1.6 |
| Bladder | 3.3 |
| Trachea | 11.8 |
| Kidney | 9.2 |
| Kidney (fetal) | 6.4 |
| Renal ca. 786-0 | 1.8 |
| Renal ca. A498 | 3.3 |
| Renal ca. RXF 393 | 1.6 |
| Renal ca. ACHN | 2.2 |
| Renal ca. UO-31 | 1.2 |
| Renal ca. TK-10 | 1.0 |
| Liver | 1.0 |
| Liver (fetal) | 2.7 |
| Liver ca. (hepatoblast) HepG2 | 1.4 |
| Lung | 6.4 |
| Lung (fetal) | 4.9 |
| Lung ca. (small cell) LX-1 | 0.4 |
| Lung ca. (small cell) NCI-H69 | 0.2 |
| Lung ca. (s. cell var.) SHP-77 | 1.6 |
| Lung ca. (large cell)NCI-H460 | 0.6 |
| Lung ca. (non-sm. cell) A549 | 0.6 |
| Lung ca. (non-s. cell) NCI-H23 | 4.8 |
| Lung ca. (non-s. cell) HOP-62 | 4.3 |
| Lung ca. (non-s. cl) NCI-H522 | 0.5 |
| Lung ca. (squam.) SW 900 | 3.5 |
| Lung ca. (squam.) NCI-H596 | 0.2 |
| Mammary gland | 5.1 |
| Breast ca.* (pl. ef) MCF-7 | 3.3 |
| Breast ca.* (pl. ef) MDA-MB-231 | 0.9 |
| Breast ca.* (pl. ef) T47D | 3.2 |
| Breast ca. BT-549 | 2.2 |
| Breast ca. MDA-N | 0.5 |
| Ovary | 40.1 |
| Ovarian ca. OVCAR-3 | 2.5 |
| Ovarian ca. OVCAR-4 | 0.4 |
| Ovarian ca. OVCAR-5 | 1.7 |
| Ovarian ca. OVCAR-8 | 1.3 |
| Ovarian ca. IGROV-1 | 1.1 |
| Ovarian ca.* (ascites) SK-OV-3 | 0.5 |
| Uterus | 4.6 |
| Placenta | 5.1 |
| Prostate | 5.4 |
| Prostate ca.* (bone met)PC-3 | 1.6 |
| Testis | 8.6 |
| Melanoma Hs688(A).T | 1.5 |
| Melanoma* (met) Hs688(B).T | 2.3 |
| Melanoma UACC-62 | 0.8 |
| Melanoma M14 | 0.4 |
| Melanoma LOX IMVI | 0.1 |
| Melanoma* (met) SK-MEL-5 | 1.3 |
| Adipose | 6.4 |

Column A - Rel. Exp. (%) Ag2779, Run 164024255

TABLE SD
Panel 2D

| Tissue Name | A |
|---|---|
| Normal Colon | 33.4 |
| CC Well to Mod Diff (ODO3866) | 4.2 |
| CC Margin (ODO3866) | 10.7 |
| CC Gr.2 rectosigmoid (ODO3868) | 6.0 |
| CC Margin (ODO3868) | 7.5 |
| CC Mod Diff (ODO3920) | 5.8 |
| CC Margin (ODO3920) | 18.2 |
| CC Gr.2 ascend colon (ODO3921) | 15.9 |
| CC Margin (ODO3921) | 9.9 |
| CC from Partial Hepatectomy (ODO4309) Mets | 22.4 |
| Liver Margin (ODO4309) | 9.9 |
| Colon mets to lung (OD04451-01) | 15.1 |
| Lung Margin (OD04451-02) | 11.7 |
| Normal Prostate 6546-1 | 100.0 |
| Prostate Cancer (OD04410) | 14.0 |
| Prostate Margin (OD04410) | 30.8 |
| Prostate Cancer (OD04720-01) | 25.0 |
| Prostate Margin (OD04720-02) | 28.9 |
| Normal Lung 061010 | 30.6 |
| Lung Met to Muscle (ODO4286) | 12.2 |
| Muscle Margin (ODO4286) | 52.9 |
| Lung Malignant Cancer (OD03126) | 71.2 |
| Lung Margin (OD03126) | 23.0 |
| Lung Cancer (OD04404) | 11.9 |
| Lung Margin (OD04404) | 22.8 |
| Lung Cancer (OD04565) | 17.8 |
| Lung Margin (OD04565) | 15.4 |
| Lung Cancer (OD04237-01) | 24.0 |
| Lung Margin (OD04237-02) | 16.0 |
| Ocular Mel Met to Liver (ODO4310) | 13.8 |
| Liver Margin (ODO4310) | 9.8 |
| Melanoma Mets to Lung (OD04321) | 4.4 |
| Lung Margin (OD04321) | 25.9 |
| Normal Kidney | 31.6 |
| Kidney Ca, Nuclear grade 2 (OD04338) | 53.2 |
| Kidney Margin (OD04338) | 23.8 |
| Kidney Ca Nuclear grade 1/2 (OD04339) | 52.1 |
| Kidney Margin (OD04339) | 32.8 |
| Kidney Ca, Clear cell type (OD04340) | 40.6 |
| Kidney Margin (OD04340) | 33.7 |
| Kidney Ca, Nuclear grade 3 (OD04348) | 10.7 |
| Kidney Margin (OD04348) | 16.0 |
| Kidney Cancer (OD04622-01) | 32.5 |
| Kidney Margin (OD04622-03) | 10.9 |
| Kidney Cancer (OD04450-01) | 27.5 |
| Kidney Margin (OD04450-03) | 25.0 |
| Kidney Cancer 8120607 | 17.4 |
| Kidney Margin 8120608 | 23.3 |
| Kidney Cancer 8120613 | 47.3 |
| Kidney Margin 8120614 | 15.6 |
| Kidney Cancer 9010320 | 15.2 |
| Kidney Margin 9010321 | 32.1 |
| Normal Uterus | 13.5 |
| Uterus Cancer 064011 | 21.2 |
| Normal Thyroid | 41.2 |
| Thyroid Cancer 064010 | 20.0 |
| Thyroid Cancer A302152 | 71.2 |
| Thyroid Margin A302153 | 32.5 |
| Normal Breast | 28.7 |
| Breast Cancer (OD04566) | 7.3 |
| Breast Cancer (OD04590-01) | 17.9 |
| Breast Cancer Mets (OD04590-03) | 27.5 |
| Breast Cancer Metastasis (OD04655-05) | 20.7 |
| Breast Cancer 064006 | 22.7 |
| Breast Cancer 1024 | 29.5 |
| Breast Cancer 9100266 | 32.5 |
| Breast Margin 9100265 | 17.9 |
| Breast Cancer A209073 | 21.8 |
| Breast Margin A209073 | 11.5 |
| Normal Liver | 6.5 |
| Liver Cancer 064003 | 6.3 |
| Liver Cancer 1025 | 8.1 |
| Liver Cancer 1026 | 13.6 |
| Liver Cancer 6004-T | 7.0 |
| Liver Tissue 6004-N | 12.2 |
| Liver Cancer 6005-T | 10.6 |
| Liver Tissue 6005-N | 4.0 |
| Normal Bladder | 15.4 |
| Bladder Cancer 1023 | 6.6 |
| Bladder Cancer A302173 | 4.9 |
| Bladder Cancer (OD04718-01) | 11.2 |
| Bladder Normal Adjacent (OD04718-03) | 28.5 |
| Normal Ovary | 32.1 |
| Ovarian Cancer 064008 | 26.8 |
| Ovarian Cancer (OD04768-07) | 37.4 |
| Ovary Margin (OD04768-08) | 16.7 |
| Normal Stomach | 35.6 |
| Gastric Cancer 9060358 | 9.1 |
| Stomach Margin 9060359 | 24.8 |
| Gastric Cancer 9060395 | 28.3 |
| Stomach Margin 9060394 | 26.4 |
| Gastric Cancer 9060397 | 15.8 |
| Stomach Margin 9060396 | 10.6 |
| Gastric Cancer 064005 | 21.0 |

Column A - Rel. Exp. (%) Ag2779, Run 162555295

TABLE SE
Panel 4D

| Tissue Name | A |
|---|---|
| Secondary Th1 act | 13.4 |
| Secondary Th2 act | 8.4 |
| Secondary Tr1 act | 11.0 |
| Secondary Th1 rest | 37.1 |
| Secondary Th2 rest | 49.0 |
| Secondary Tr1 rest | 49.3 |
| Primary Th1 act | 4.7 |
| Primary Th2 act | 1.9 |
| Primary Tr1 act | 9.0 |
| Primary Th1 rest | 91.4 |
| Primary Th2 rest | 81.8 |
| Primary Tr1 rest | 35.6 |
| CD45RA CD4 lymphocyte act | 4.1 |
| CD45RO CD4 lymphocyte act | 15.6 |
| CD8 lymphocyte act | 11.9 |
| Secondary CD8 lymphocyte rest | 9.7 |
| Secondary CD8 lymphocyte act | 8.6 |
| CD4 lymphocyte none | 49.0 |
| 2ry Th1/Th2/Tr1_anti-CD95 CH11 | 89.5 |
| LAK cells rest | 17.3 |
| LAK cells IL-2 | 36.1 |
| LAK cells IL-2 + IL-12 | 17.1 |
| LAK cells IL-2 + IFN gamma | 28.7 |
| LAK cells IL-2 + IL-18 | 23.5 |
| LAK cells PMA/ionomycin | 4.9 |
| NK Cells IL-2 rest | 26.2 |
| Two Way MLR 3 day | 31.9 |
| Two Way MLR 5 day | 15.4 |
| Two Way MLR 7 day | 27.4 |
| PBMC rest | 39.8 |
| PBMC PWM | 29.5 |
| PBMC PHA-L | 16.5 |
| Ramos (B cell) none | 5.8 |
| Ramos (B cell) ionomycin | 14.1 |
| B lymphocytes PWM | 25.9 |
| B lymphocytes CD40L and IL-4 | 36.1 |
| EOL-1 dbcAMP | 9.8 |
| EOL-1 dbcAMP PMA/ionomycin | 4.7 |
| Dendritic cells none | 12.1 |
| Dendritic cells LPS | 9.9 |
| Dendritic cells anti-CD40 | 9.0 |
| Monocytes rest | 12.9 |
| Monocytes LPS | 3.7 |
| Macrophages rest | 10.2 |
| Macrophages LPS | 5.5 |
| HUVEC none | 17.1 |
| HUVEC starved | 29.3 |
| HUVEC IL-1beta | 4.3 |
| HUVEC IFN gamma | 23.0 |
| HUVEC TNF alpha + IFN gamma | 4.2 |
| HUVEC TNF alpha + IL4 | 7.1 |
| HUVEC IL-11 | 18.4 |
| Lung Microvascular EC none | 18.2 |
| Lung Microvascular EC TNFalpha + IL-1beta | 15.1 |
| Microvascular Dermal EC none | 29.5 |
| Microsvasular Dermal EC TNFalpha + IL-1beta | 9.4 |
| Bronchial epithelium TNFalpha + IL1beta | 20.4 |
| Small airway epithelium none | 5.0 |
| Small airway epithelium TNFalpha + IL-1beta | 21.0 |
| Coronery artery SMC rest | 24.3 |
| Coronery artery SMC TNFalpha + IL-1beta | 18.3 |
| Astrocytes rest | 34.2 |
| Astrocytes TNFalpha + IL-1beta | 29.1 |
| KU-812 (Basophil) rest | 5.8 |
| KU-812 (Basophil) PMA/ionomycin | 10.5 |
| CCD1106 (Keratinocytes) none | 4.7 |
| CCD1106 (Keratinocytes) TNFalpha + IL-1beta | 4.3 |
| Liver cirrhosis | 7.6 |
| Lupus kidney | 11.5 |
| NCI-H292 none | 15.3 |
| NCI-H292 IL-4 | 8.9 |
| NCI-H292 IL-9 | 11.0 |
| NCI-H292 IL-13 | 6.3 |
| NCI-H292 IFN gamma | 6.0 |
| HPAEC none | 26.2 |
| HPAEC TNF alpha + IL-1 beta | 11.5 |
| Lung fibroblast none | 27.4 |
| Lung fibroblast TNF alpha + IL-1 beta | 66.0 |
| Lung fibroblast IL-4 | 25.2 |
| Lung fibroblast IL-9 | 22.4 |
| Lung fibroblast IL-13 | 18.3 |
| Lung fibroblast IFN gamma | 34.9 |
| Dermal fibroblast CCD1070 rest | 8.8 |
| Dermal fibroblast CCD1070 TNF alpha | 49.3 |
| Dermal fibroblast CCD1070 IL-1 beta | 4.9 |
| Dermal fibroblast IFN gamma | 4.9 |
| Dermal fibroblast IL-4 | 11.2 |
| IBD Colitis 2 | 5.1 |
| IBD Crohn's | 11.1 |
| Colon | 42.6 |
| Lung | 26.8 |
| Thymus | 41.5 |
| Kidney | 100.0 |

Column A - Rel. Exp. (%) Ag2779, Run 162188258

TABLE SF
Panel 5D

| Tissue Name | A |
|---|---|
| 97457_Patient-02go_adipose | 30.1 |
| 97476_Patient-07sk_skeletal muscle | 13.8 |
| 97477_Patient-07ut_uterus | 31.0 |
| 97478_Patient-07pl_placenta | 17.4 |
| 97481_Patient-08sk_skeletal muscle | 38.7 |
| 97482_Patient-08ut_uterus | 45.4 |
| 97483_Patient-08pl_placenta | 16.0 |
| 97486_Patient-09sk_skeletal muscle | 7.4 |
| 97487_Patient-09ut_uterus | 19.8 |
| 97488_Patient-09pl_placenta | 16.7 |
| 97492_Patient-10ut_uterus | 66.4 |
| 97493_Patient-10pl_placenta | 46.3 |
| 97495_Patient-11go_adipose | 7.2 |
| 97496_Patient-11sk_skeletal muscle | 12.4 |
| 97497_Patient-11ut_uterus | 68.3 |
| 97498_Patient-11pl_placenta | 26.4 |
| 97500_Patient-12go_adipose | 35.8 |
| 97501_Patient-12sk_skeletal muscle | 78.5 |
| 97502_Patient-12ut_uterus | 100.0 |
| 97503_Patient-12pl_placenta | 21.5 |
| 94721_Donor 2 U - A_Mesenchymal Stem Cells | 23.3 |
| 94722_Donor 2 U - B_Mesenchymal Stem Cells | 28.1 |
| 94723_Donor 2 U - C_Mesenchymal Stem Cells | 20.0 |
| 94709_Donor 2 AM - A_adipose | 16.0 |
| 94710_Donor 2 AM - B_adipose | 15.1 |
| 94711_Donor 2 AM - C_adipose | 7.9 |
| 94712_Donor 2 AD - A_adipose | 32.1 |
| 94713_Donor 2 AD - B_adipose | 40.1 |
| 94714_Donor 2 AD - C_adipose | 35.1 |
| 94742_Donor 3 U - A_Mesenchymal Stem Cells | 10.1 |
| 94743_Donor 3 U - B_Mesenchymal Stem Cells | 14.1 |
| 94730_Donor 3 AM - A_adipose | 13.5 |
| 94731_Donor 3 AM - B_adipose | 8.8 |
| 94732_Donor 3 AM - C_adipose | 5.2 |
| 94733_Donor 3 AD - A_adipose | 26.6 |
| 94734_Donor 3 AD - B_adipose | 17.4 |
| 94735_Donor 3 AD - C_adipose | 26.1 |
| 77138_Liver_HepG2untreated | 25.0 |
| 73556_Heart_Cardiac stromal cells (primary) | 7.3 |
| 81735_Small Intestine | 55.5 |
| 72409_Kidney_Proximal Convoluted Tubule | 4.2 |
| 82685_Small intestine_Duodenum | 21.2 |
| 90650_Adrenal_Adrenocortical adenoma | 30.1 |
| 72410_Kidney_HRCE | 25.7 |
| 72411_Kidney_HRE | 14.3 |
| 73139_Uterus_Uterine smooth muscle cells | 14.7 |

Column A - Rel. Exp. (%) Ag2779, Run 255179443

AI_comprehensive panel_v1.0 Summary: Ag2779 Highest expression of this gene was detected in normal lung (CT=27). Moderate levels of expression of this gene were detected in samples derived from osteoarthritic (OA) bone and normal adjacent bone as well as OA cartilage, OA synovium and OA synovial fluid samples. Moderate to low levels of expression were also detected in cartilage, bone, synovium and synovial fluid samples from rheumatoid arthritis patients as well as in samples derived from normal lung samples, COPD lung, emphysema, atopic asthma, asthma, allergy, Crohn's disease (normal matched control and diseased), ulcerative colitis(normal matched control and diseased), and psoriasis (normal matched control and diseased). Therapeutic modulation of this gene or gene product is useful for the amelioration of symptoms/conditions associated with autoimmune and inflammatory disorders including psoriasis, allergy, asthma, inflammatory bowel disease, rheumatoid arthritis and osteoarthritis.

Panel 1.3D Summary: Ag2779 Highest expression was seen in fetal skeletal muscle (CT=25). Prominent expression was seen in normal tissues including ovary and bone marrow.

Panel 2D Summary: Ag2779 Highest expression was seen in prostate (CT=23.5). High levels of expression were seen in most of the samples on this panel, showing that this gene or its protein product plays a role in cell growth and/or proliferation.

Panel 4D Summary: Ag2779 Highest expression was seen in the kidney (CT=26.7). In addition, this gene was more highly expressed in resting T cells (Th1, Th2, Tr1) than in activated T cells. Thus, this gene is be involved in T lymphocyte function. Therapeutics targeting this gene or its protein product are useful for the stimulation of their activity and act as anti-inflammatory therapeutic for T cell-mediated autoimmune and inflammatory diseases.

Panel SD Summary: Ag2779 Highest expression was seen in normal uterus (CT=27), with prominent expression in normal adipose and placenta. This strong expression in normal tissues was in agreement with the results in Panel 1.3D.

T. CG52979-03 and CG52988-02 and CG52988-03: GAGE

Expression of gene CG52979-03, variant CG52988-02 and full-length physical clone CG52988-03 was assessed using the primer-probe sets Ag436b, and Ag436, described in Tables TA and TB. Results of the RTQ-PCR runs are shown in Tables TC, TD, TE, TF, TG and TH.

TABLE TA
Probe Name Ag436b

| | | | | |
|---|---|---|---|---|
| Primers | Sequences | Length | Start Position | SEQ ID No |

| | | | | |
|---|---|---|---|---|
| Forward | 5′-gagcctggagatggtcctga-3′ | 20 | 184 | 1339 |
| Probe | TET-5′-agatcaaaagtgggcataataccctccctga-3′-TAMRA | 31 | 151 | 1340 |
| Reverse | 5′-atcacctgcttccagcactttagt-3′ | 24 | 126 | 1341 |

TABLE TB
Probe Name Ag436

| | | | | |
|---|---|---|---|---|
| Primers | Sequences | Length | Start Position | SEQ ID No |

| | | | | |
|---|---|---|---|---|
| Forward | 5′-gcctggagatggtcctgatg-3′ | 20 | 182 | 1342 |
| Probe | TET-5′-tgagatcaaaagtgggcataataccctccct-3′-TAMRA | 31 | 149 | 1343 |
| Reverse | 5′-catcacctgcttccagcactt-3′ | 21 | 125 | 1344 |

TABLE TC
Panel 1

| Tissue Name | A |
|---|---|
| Endothelial cells | 0.0 |
| Endothelial cells (treated) | 0.0 |
| Pancreas | 1.6 |
| Pancreatic ca. CAPAN 2 | 0.0 |
| Adrenal gland | 0.4 |
| Thyroid | 0.0 |
| Salivary gland | 0.1 |
| Pituitary gland | 0.0 |
| Brain (fetal) | 0.0 |
| Brain (whole) | 0.0 |
| Brain (amygdala) | 0.0 |
| Brain (cerebellum) | 0.0 |
| Brain (hippocampus) | 0.1 |
| Brain (substantia nigra) | 0.1 |
| Brain (thalamus) | 0.0 |
| Brain (hypothalamus) | 0.4 |
| Spinal cord | 0.3 |
| glio/astro U87-MG | 0.0 |
| glio/astro U-118-MG | 0.9 |
| astrocytoma SW1783 | 0.0 |
| neuro*; met SK-N-AS | 0.3 |
| astrocytoma SF-539 | 0.0 |
| astrocytoma SNB-75 | 0.0 |
| glioma SNB-19 | 0.1 |
| glioma U251 | 0.1 |
| glioma SF-295 | 4.5 |
| Heart | 0.1 |
| Skeletal muscle | 0.0 |
| Bone marrow | 6.1 |
| Thymus | 1.0 |
| Spleen | 0.8 |
| Lymph node | 1.4 |
| Colon (ascending) | 3.3 |
| Stomach | 0.3 |
| Small intestine | 0.2 |
| Colon ca. SW480 | 0.1 |
| Colon ca.* SW620 (SW480 met) | 0.1 |
| Colon ca. HT29 | 0.0 |
| Colon ca. HCT-116 | 0.1 |
| Colon ca. CaCo-2 | 0.0 |
| Colon ca. HCT-15 | 0.2 |
| Colon ca. HCC-2998 | 0.2 |
| Gastric ca.* (liver met) NCI-N87 | 0.2 |
| Bladder | 0.0 |
| Trachea | 0.2 |
| Kidney | 0.2 |
| Kidney (fetal) | 0.3 |
| Renal ca. 786-0 | 0.0 |
| Renal ca. A498 | 0.0 |
| Renal ca. RXF 393 | 0.0 |
| Renal ca. ACHN | 0.0 |
| Renal ca. UO-31 | 0.0 |
| Renal ca. TK-10 | 0.0 |
| Liver | 0.4 |
| Liver (fetal) | 4.6 |
| Liver ca. (hepatoblast) HepG2 | 0.3 |
| Lung | 0.0 |
| Lung (fetal) | 0.0 |
| Lung ca. (small cell) LX-1 | 1.3 |
| Lung ca. (small cell) NCI-H69 | 0.8 |
| Lung ca. (s. cell var.) SHP-77 | 0.0 |
| Lung ca. (large cell)NCI-H460 | 1.0 |
| Lung ca. (non-sm. cell) A549 | 0.5 |
| Lung ca. (non-s. cell) NCI-H23 | 1.0 |
| Lung ca. (non-s. cell) HOP-62 | 0.2 |
| Lung ca. (non-s. cl) NCI-H522 | 0.3 |
| Lung ca. (squam.) SW 900 | 0.0 |
| Lung ca. (squam.) NCI-H596 | 0.2 |
| Mammary gland | 1.3 |
| Breast ca.* (pl. ef) MCF-7 | 0.0 |
| Breast ca.* (pl. ef) MDA-MB-231 | 0.5 |
| Breast ca.* (pl. ef) T47D | 0.5 |
| Breast ca. BT-549 | 0.0 |
| Breast ca. MDA-N | 100.0 |
| Ovary | 0.0 |
| Ovarian ca. OVCAR-3 | 1.3 |
| Ovarian ca. OVCAR-4 | 0.0 |
| Ovarian ca. OVCAR-5 | 0.0 |
| Ovarian ca. OVCAR-8 | 3.5 |
| Ovarian ca. IGROV-1 | 0.0 |
| Ovarian ca. (ascites) SK-OV-3 | 0.0 |
| Uterus | 0.1 |
| Placenta | 13.3 |
| Prostate | 0.4 |
| Prostate ca.* (bone met) PC-3 | 0.9 |
| Testis | 79.0 |
| Melanoma Hs688(A).T | 0.0 |
| Melanoma* (met) Hs688(B).T | 0.0 |
| Melanoma UACC-62 | 0.0 |
| Melanoma M14 | 0.1 |
| Melanoma LOX IMVI | 0.0 |
| Melanoma* (met) SK-MEL-5 | 0.0 |
| Melanoma SK-MEL-28 | 8.7 |

Column A - Rel. Exp. (%) Ag436, Run 109664430

TABLE TD
Panel 1.1

| Tissue Name | A | B |
|---|---|---|
| Adrenal gland | 0.0 | 0.0 |
| Bladder | 0.1 | 0.0 |
| Brain (amygdala) | 0.0 | 0.0 |
| Brain (cerebellum) | 0.4 | 0.0 |
| Brain (hippocampus) | 0.0 | 0.0 |
| Brain (substantia nigra) | 0.4 | 0.0 |
| Brain (thalamus) | 0.0 | 0.0 |
| Cerebral Cortex | 0.0 | 0.0 |
| Brain (fetal) | 0.0 | 0.0 |
| Brain (whole) | 0.4 | 0.0 |
| glio/astro U-118-MG | 0.8 | 0.1 |
| astrocytoma SF-539 | 0.0 | 0.0 |
| astrocytoma SNB-75 | 0.0 | 0.0 |
| astrocytoma SW1783 | 0.0 | 0.0 |
| glioma U251 | 0.1 | 0.0 |
| glioma SF-295 | 3.3 | 4.2 |
| glioma SNB-19 | 0.2 | 0.0 |
| glio/astro U87-MG | 0.0 | 0.0 |
| neuro*; met SK-N-AS | 0.2 | 0.0 |
| Mammary gland | 0.1 | 0.0 |
| Breast ca. BT-549 | 0.0 | 0.0 |
| Breast ca. MDA-N | 100.0 | 100.0 |
| Breast ca.* (pl. ef) T47D | 0.4 | 0.0 |
| Breast ca.* (pl. ef) MCF-7 | 0.0 | 0.0 |
| Breast ca.* (pl. ef) MDA-MB-231 | 0.0 | 0.0 |
| Small intestine | 0.3 | 0.0 |
| Colorectal | 0.0 | 0.0 |
| Colon ca. HT29 | 0.3 | 0.0 |
| Colon ca. CaCo-2 | 0.1 | 0.0 |
| Colon ca. HCT-15 | 0.1 | 0.0 |
| Colon ca. HCT-116 | 0.1 | 0.0 |
| Colon ca. HCC-2998 | 0.2 | 0.0 |
| Colon ca. SW480 | 0.1 | 0.0 |
| Colon ca.* SW620 (SW480 met) | 0.4 | 0.0 |
| Stomach | 0.4 | 0.0 |
| Gastric ca. (liver met) NCI-N87 | 0.4 | 0.0 |
| Heart | 0.0 | 0.0 |
| Skeletal muscle (Fetal) | 0.0 | 0.0 |
| Skeletal muscle | 0.0 | 0.0 |
| Endothelial cells | 0.0 | 0.0 |
| Heart (Fetal) | 0.0 | 0.0 |
| Kidney | 0.3 | 0.0 |
| Kidney (fetal) | 0.2 | 0.0 |
| Renal ca. 786-0 | 0.0 | 0.0 |
| Renal ca. A498 | 0.0 | 0.0 |
| Renal ca. ACHN | 0.0 | 0.0 |
| Renal ca. TK-10 | 0.0 | 0.0 |
| Renal ca. UO-31 | 0.0 | 0.0 |
| Renal ca. RXF 393 | 0.0 | 0.0 |
| Liver | 0.6 | 0.0 |
| Liver (fetal) | 1.5 | 1.7 |
| Liver ca. (hepatoblast) HepG2 | 0.2 | 0.0 |
| Lung | 0.0 | 0.0 |
| Lung (fetal) | 0.3 | 0.0 |
| Lung ca. (non-s. cell) HOP-62 | 0.4 | 0.2 |
| Lung ca. (large cell)NCI-H460 | 0.3 | 0.0 |
| Lung ca. (non-s. cell) NCI-H23 | 0.4 | 0.0 |
| Lung ca. (non-s. cl) NCI-H522 | 1.0 | 0.3 |
| Lung ca. (non-sm. cell) A549 | 0.2 | 0.0 |
| Lung ca. (s. cell var.) SHP-77 | 0.2 | 0.0 |
| Lung ca. (small cell) LX-1 | 2.3 | 0.0 |
| Lung ca. (small cell) NCI-H69 | 0.9 | 0.0 |
| Lung ca. (squam.) SW 900 | 0.0 | 0.0 |
| Lung ca. (squam.) NCI-H596 | 0.2 | 0.0 |
| Lymph node | 0.4 | 0.0 |
| Spleen | 0.2 | 0.0 |
| Thymus | 0.4 | 0.0 |
| Ovary | 0.0 | 0.0 |
| Ovarian ca. IGROV-1 | 0.1 | 0.0 |
| Ovarian ca. OVCAR-3 | 0.9 | 0.3 |
| Ovarian ca. OVCAR-4 | 0.0 | 0.0 |
| Ovarian ca. OVCAR-5 | 0.4 | 0.0 |
| Ovarian ca. OVCAR-8 | 0.9 | 0.2 |
| Ovarian ca.* (ascites) SK-OV-3 | 0.0 | 0.0 |
| Pancreas | 2.4 | 5.1 |
| Pancreatic ca. CAPAN 2 | 0.0 | 0.0 |
| Pituitary gland | 0.2 | 0.0 |
| Placenta | 4.2 | 3.8 |
| Prostate | 0.5 | 0.0 |
| Prostate ca.* (bone met) PC-3 | 0.3 | 0.0 |
| Salivary gland | 0.2 | 0.0 |
| Trachea | 0.0 | 0.0 |
| Spinal cord | 0.2 | 0.0 |
| Testis | 44.4 | 28.7 |
| Thyroid | 0.0 | 0.0 |
| Uterus | 0.0 | 0.0 |
| Melanoma M14 | 0.2 | 0.0 |
| Melanoma LOX IMVI | 0.0 | 0.0 |
| Melanoma UACC-62 | 0.0 | 0.0 |
| Melanoma SK-MEL-28 | 0.1 | 0.0 |
| Melanoma* (met) SK-MEL-5 | 0.0 | 0.0 |
| Melanoma Hs688(A).T | 0.0 | 0.0 |
| Melanoma* (met) Hs688(B).T | 0.0 | 0.0 |

Column A - Rel. Exp. (%) Ag436, Run 109665004
Column B - Rel. Exp. (%) Ag436b, Run 111165634

TABLE TE
Panel 1.3D

| Tissue Name | A |
|---|---|
| Liver adenocarcinoma | 2.5 |
| Pancreas | 0.8 |
| Pancreatic ca. CAPAN 2 | 0.0 |
| Adrenal gland | 0.0 |
| Thyroid | 0.5 |
| Salivary gland | 0.0 |
| Pituitary gland | 0.6 |
| Brain (fetal) | 3.5 |
| Brain (whole) | 1.6 |
| Brain (amygdala) | 0.0 |
| Brain (cerebellum) | 2.2 |
| Brain (hippocampus) | 2.9 |
| Brain (substantia nigra) | 2.1 |
| Brain (thalamus) | 0.7 |
| Cerebral Cortex | 1.4 |
| Spinal cord | 0.0 |
| glio/astro U87-MG | 1.0 |
| glio/astro U-118-MG | 5.1 |
| astrocytoma SW1783 | 4.3 |
| neuro*; met SK-N-AS | 0.7 |
| astrocytoma SF-539 | 0.0 |
| astrocytoma SNB-75 | 0.0 |
| glioma SNB-19 | 0.8 |
| glioma U251 | 0.3 |
| glioma SF-295 | 5.6 |
| Heart (fetal) | 0.8 |
| Heart | 0.0 |
| Skeletal muscle (fetal) | 0.0 |
| Skeletal muscle | 0.0 |
| Bone marrow | 16.7 |
| Thymus | 0.7 |
| Spleen | 1.4 |
| Lymph node | 3.7 |
| Colorectal | 1.1 |
| Stomach | 1.1 |
| Small intestine | 1.3 |
| Colon ca. SW480 | 0.7 |
| Colon ca.* SW620(SW480 met) | 1.0 |
| Colon ca. HT29 | 2.0 |
| Colon ca. HCT-116 | 0.0 |
| Colon ca. CaCo-2 | 0.7 |
| Colon ca. tissue(ODO3866) | 0.0 |
| Colon ca. HCC-2998 | 0.7 |
| Gastric ca.* (liver met) NCI-N87 | 0.0 |
| Bladder | 0.0 |
| Trachea | 0.0 |
| Kidney | 0.8 |
| Kidney (fetal) | 3.3 |
| Renal ca. 786-0 | 1.0 |
| Renal ca. A498 | 1.0 |
| Renal ca. RXF 393 | 1.7 |
| Renal ca. ACHN | 0.0 |
| Renal ca. UO-31 | 0.0 |
| Renal ca. TK-10 | 0.0 |
| Liver | 2.2 |
| Liver (fetal) | 11.6 |
| Liver ca. (hepatoblast) HepG2 | 0.0 |
| Lung | 2.7 |
| Lung (fetal) | 4.0 |
| Lung ca. (small cell) LX-1 | 2.5 |
| Lung ca. (small cell) NCI-H69 | 0.0 |
| Lung ca. (s. cell var.) SHP-77 | 0.0 |
| Lung ca. (large cell)NCI-H460 | 0.0 |
| Lung ca. (non-sm. cell) A549 | 8.4 |
| Lung ca. (non-s. cell) NCI-H23 | 2.8 |
| Lung ca. (non-s. cell) HOP-62 | 0.9 |
| Lung ca. (non-s. cl) NCI-H522 | 0.0 |
| Lung ca. (squam.) SW 900 | 1.0 |
| Lung ca. (squam.) NCI-H596 | 0.0 |
| Mammary gland | 0.0 |
| Breast ca.* (pl. ef) MCF-7 | 0.0 |
| Breast ca.* (pl. ef) MDA-MB-231 | 1.7 |
| Breast ca.* (pl. ef) T47D | 0.0 |
| Breast ca. BT-549 | 0.0 |
| Breast ca. MDA-N | 76.8 |
| Ovary | 0.0 |
| Ovarian ca. OVCAR-3 | 3.1 |
| Ovarian ca. OVCAR-4 | 0.0 |
| Ovarian ca. OVCAR-5 | 0.8 |
| Ovarian ca. OVCAR-8 | 2.5 |
| Ovarian ca. IGROV-1 | 0.9 |
| Ovarian ca.* (ascites) SK-OV-3 | 1.7 |
| Uterus | 0.0 |
| Placenta | 6.3 |
| Prostate | 1.3 |
| Prostate ca.* (bone met)PC-3 | 1.7 |
| Testis | 100.0 |
| Melanoma Hs688(A).T | 0.0 |
| Melanoma* (met) Hs688(B).T | 0.9 |
| Melanoma UACC-62 | 0.0 |
| Melanoma M14 | 0.0 |
| Melanoma LOX IMVI | 0.0 |
| Melanoma* (met) SK-MEL-5 | 2.0 |
| Adipose | 2.5 |

Column A - Rel. Exp. (%) Ag436, Run 153954656

TABLE TF
Panel 2D

| Tissue Name | A |
|---|---|
| Normal Colon | 0.0 |
| CC Well to Mod Diff (ODO3866) | 0.9 |
| CC Margin (ODO3866) | 0.9 |
| CC Gr.2 rectosigmoid (ODO3868) | 0.0 |
| CC Margin (ODO3868) | 0.8 |
| CC Mod Diff (ODO3920) | 0.5 |
| CC Margin (ODO3920) | 0.4 |
| CC Gr.2 ascend colon (ODO3921) | 0.9 |
| CC Margin (ODO3921) | 0.5 |
| CC from Partial Hepatectomy (ODO4309) Mets | 1.7 |
| Liver Margin (ODO4309) | 0.8 |
| Colon mets to lung (OD04451-01) | 0.8 |
| Lung Margin (OD04451-02) | 0.0 |
| Normal Prostate 6546-1 | 1.4 |
| Prostate Cancer (OD04410) | 1.5 |
| Prostate Margin (OD04410) | 0.3 |
| Prostate Cancer (OD04720-01) | 1.6 |
| Prostate Margin (OD04720-02) | 1.0 |
| Normal Lung 061010 | 1.4 |
| Lung Met to Muscle (ODO4286) | 0.6 |
| Muscle Margin (ODO4286) | 0.7 |
| Lung Malignant Cancer (OD03126) | 0.0 |
| Lung Margin (OD03126) | 0.6 |
| Lung Cancer (OD04404) | 0.0 |
| Lung Margin (OD04404) | 0.0 |
| Lung Cancer (OD04565) | 0.6 |
| Lung Margin (OD04565) | 0.7 |
| Lung Cancer (OD04237-01) | 0.9 |
| Lung Margin (OD04237-02) | 0.2 |
| Ocular Mel Met to Liver (ODO4310) | 0.0 |
| Liver Margin (ODO4310) | 0.2 |
| Melanoma Mets to Lung (OD04321) | 0.6 |
| Lung Margin (OD04321) | 0.4 |
| Normal Kidney | 1.0 |
| Kidney Ca, Nuclear grade 2 (OD04338) | 0.2 |
| Kidney Margin (OD04338) | 2.0 |
| Kidney Ca Nuclear grade 1/2 (OD04339) | 0.4 |
| Kidney Margin (OD04339) | 0.6 |
| Kidney Ca, Clear cell type (OD04340) | 0.4 |
| Kidney Margin (OD04340) | 0.2 |
| Kidney Ca, Nuclear grade 3 (OD04348) | 0.3 |
| Kidney Margin (OD04348) | 0.0 |
| Kidney Cancer (OD04622-01) | 0.3 |
| Kidney Margin (OD04622-03) | 0.0 |
| Kidney Cancer (OD04450-01) | 0.0 |
| Kidney Margin (OD04450-03) | 0.5 |
| Kidney Cancer 8120607 | 0.0 |
| Kidney Margin 8120608 | 0.2 |
| Kidney Cancer 8120613 | 0.2 |
| Kidney Margin 8120614 | 0.0 |
| Kidney Cancer 9010320 | 56.6 |
| Kidney Margin 9010321 | 0.0 |
| Normal Uterus | 0.0 |
| Uterus Cancer 064011 | 0.7 |
| Normal Thyroid | 0.4 |
| Thyroid Cancer 064010 | 0.0 |
| Thyroid Cancer A302152 | 0.3 |
| Thyroid Margin A302153 | 0.3 |
| Normal Breast | 0.4 |
| Breast Cancer (OD04566) | 1.3 |
| Breast Cancer (OD04590-01) | 1.1 |
| Breast Cancer Mets (OD04590-03) | 2.7 |
| Breast Cancer Metastasis (OD04655-05) | 3.5 |
| Breast Cancer 064006 | 37.9 |
| Breast Cancer 1024 | 1.1 |
| Breast Cancer 9100266 | 0.4 |
| Breast Margin 9100265 | 0.4 |
| Breast Cancer A209073 | 1.0 |
| Breast Margin A209073 | 0.9 |
| Normal Liver | 0.0 |
| Liver Cancer 064003 | 0.3 |
| Liver Cancer 1025 | 0.0 |
| Liver Cancer 1026 | 0.0 |
| Liver Cancer 6004-T | 0.2 |
| Liver Tissue 6004-N | 1.0 |
| Liver Cancer 6005-T | 0.2 |
| Liver Tissue 6005-N | 0.6 |
| Normal Bladder | 0.3 |
| Bladder Cancer 1023 | 0.0 |
| Bladder Cancer A302173 | 100.0 |
| Bladder Cancer (OD04718-01) | 2.8 |
| Bladder Normal Adjacent (OD04718-03) | 0.6 |
| Normal Ovary | 0.0 |
| Ovarian Cancer 064008 | 1.0 |
| Ovarian Cancer (OD04768-07) | 2.1 |
| Ovary Margin (OD04768-08) | 0.6 |
| Normal Stomach | 0.9 |
| Gastric Cancer 9060358 | 0.3 |
| Stomach Margin 9060359 | 0.8 |
| Gastric Cancer 9060395 | 0.5 |
| Stomach Margin 9060394 | 0.0 |
| Gastric Cancer 9060397 | 1.4 |
| Stomach Margin 9060396 | 0.0 |
| Gastric Cancer 064005 | 0.3 |

Column A - Rel. Exp. (%) Ag436, Run 153954657

TABLE TG
Panel 3D

| Tissue Name | A |
|---|---|
| Daoy- Medulloblastoma | 0.0 |
| TE671- Medulloblastoma | 0.0 |
| D283 Med- Medulloblastoma | 0.1 |
| PFSK-1- Primitive Neuroectodermal | 0.0 |
| XF-498- CNS | 0.2 |
| SNB-78- Glioma | 0.0 |
| SF-268- Glioblastoma | 0.0 |
| T98G- Glioblastoma | 0.1 |
| SK-N-SH- Neuroblastoma (metastasis) | 0.5 |
| SF-295- Glioblastoma | 0.5 |
| Cerebellum | 0.2 |
| Cerebellum | 0.0 |
| NCI-H292- Mucoepidermoid lung carcinoma | 0.0 |
| DMS-114- Small cell lung cancer | 0.0 |
| DMS-79- Small cell lung cancer | 0.0 |
| NCI-H146- Small cell lung cancer | 16.0 |
| NCI-H526- Small cell lung cancer | 0.1 |
| NCI-N417- Small cell lung cancer | 0.0 |
| NCI-H82- Small cell lung cancer | 0.0 |
| NCI-H157- Squamous cell lung cancer (metastasis) | 0.0 |
| NCI-H1155- Large cell lung cancer | 0.0 |
| NCI-H1299- Large cell lung cancer | 7.6 |
| NCI-H727- Lung carcinoid | 0.1 |
| NCI-UMC-11- Lung carcinoid | 0.1 |
| LX-1- Small cell lung cancer | 0.0 |
| Colo-205- Colon cancer | 0.1 |
| KM12- Colon cancer | 0.0 |
| KM20L2- Colon cancer | 0.0 |
| NCI-H716- Colon cancer | 0.0 |
| SW-48- Colon adenocarcinoma | 0.0 |
| SW1116- Colon adenocarcinoma | 0.0 |
| LS 174T- Colon adenocarcinoma | 0.1 |
| SW-948- Colon adenocarcinoma | 0.0 |
| SW-480- Colon adenocarcinoma | 0.0 |
| NCI-SNU-5- Gastric carcinoma | 0.0 |
| KATO III- Gastric carcinoma | 0.0 |
| NCI-SNU-16- Gastric carcinoma | 0.0 |
| NCI-SNU-1- Gastric carcinoma | 0.0 |
| RF-1- Gastric adenocarcinoma | 0.0 |
| RF-48- Gastric adenocarcinoma | 0.0 |
| MKN-45- Gastric carcinoma | 0.0 |
| NCI-N87- Gastric carcinoma | 0.0 |
| OVCAR-5- Ovarian carcinoma | 0.0 |
| RL95-2- Uterine carcinoma | 0.0 |
| HelaS3- Cervical adenocarcinoma | 0.0 |
| Ca Ski- Cervical epidermoid carcinoma (metastasis) | 0.0 |
| ES-2- Ovarian clear cell carcinoma | 0.0 |
| Ramos- Stimulated with PMA/ionomycin 6 h | 0.1 |
| Ramos- Stimulated with PMA/ionomycin 14 h | 0.0 |
| MEG-01- Chronic myelogenous leukemia | 0.1 |
| (megokaryoblast) | |
| Raji- Burkitt's lymphoma | 0.0 |
| Daudi- Burkitt's lymphoma | 0.0 |
| U266- B-cell plasmacytoma | 100.0 |
| CA46- Burkitt's lymphoma | 0.0 |
| RL- non-Hodgkin's B-cell lymphoma | 0.0 |
| JM1- pre-B-cell lymphoma | 0.0 |
| Jurkat- T cell leukemia | 0.0 |
| TF-1- Erythroleukemia | 0.0 |
| HUT 78- T-cell lymphoma | 0.0 |
| U937- Histiocytic lymphoma | 0.0 |
| KU-812- Myelogenous leukemia | 0.5 |
| 769-P- Clear cell renal carcinoma | 0.0 |
| Caki-2- Clear cell renal carcinoma | 0.0 |
| SW 839- Clear cell renal carcinoma | 0.0 |
| Rhabdoid kidney tumor | 0.0 |
| Hs766T- Pancreatic carcinoma (LN metastasis) | 0.0 |
| CAPAN-1- Pancreatic adenocarcinoma (liver | 0.0 |
| metastasis) | |
| SU86.86- Pancreatic carcinoma (liver | 0.0 |
| metastasis) | |
| BxPC-3- Pancreatic adenocarcinoma | 0.0 |
| HPAC- Pancreatic adenocarcinoma | 0.0 |
| MIA PaCa-2- Pancreatic carcinoma | 0.0 |
| CFPAC-1- Pancreatic ductal adenocarcinoma | 0.1 |
| PANC-1- Pancreatic epithelioid ductal | 0.0 |
| carcinoma | |
| T24- Bladder carcinma (transitional cell) | 0.0 |
| 5637- Bladder carcinoma | 0.0 |
| HT-1197- Bladder carcinoma | 0.0 |
| UM-UC-3- Bladder carcinma (transitional cell) | 0.0 |
| A204- Rhabdomyosarcoma | 0.1 |
| HT-1080- Fibrosarcoma | 0.0 |
| MG-63- Osteosarcoma | 0.0 |
| SK-LMS-1- Leiomyosarcoma (vulva) | 0.0 |
| SJRH30- Rhabdomyosarcoma (met to bone marrow) | 0.0 |
| A431- Epidermoid carcinoma | 0.0 |
| WM266-4- Melanoma | 0.0 |
| DU 145- Prostate carcinoma (brain metastasis) | 0.1 |
| MDA-MB-468- Breast adenocarcinoma | 0.1 |
| SCC-4- Squamous cell carcinoma of tongue | 0.0 |
| SCC-9- Squamous cell carcinoma of tongue | 0.0 |
| SCC-15- Squamous cell carcinoma of tongue | 0.1 |
| CAL 27- Squamous cell carcinoma of tongue | 0.0 |

Column A - Rel. Exp. (%) Ag436, Run 168238445

TABLE TH
Panel 4D

| Tissue Name | A |
|---|---|
| Secondary Th1 act | 0.0 |
| Secondary Th2 act | 0.0 |
| Secondary Tr1 act | 18.6 |
| Secondary Th1 rest | 22.7 |
| Secondary Th2 rest | 22.5 |
| Secondary Tr1 rest | 6.8 |
| Primary Th1 act | 19.5 |
| Primary Th2 act | 22.1 |
| Primary Tr1 act | 35.8 |
| Primary Th1 rest | 47.6 |
| Primary Th2 rest | 62.0 |
| Primary Tr1 rest | 12.9 |
| CD45RA CD4 lymphocyte act | 16.8 |
| CD45RO CD4 lymphocyte act | 29.9 |
| CD8 lymphocyte act | 43.2 |
| Secondary CD8 lymphocyte rest | 9.9 |
| Secondary CD8 lymphocyte act | 34.4 |
| CD4 lymphocyte none | 24.5 |
| 2ry Th1/Th2/Tr1_anti-CD95 CH11 | 26.8 |
| LAK cells rest | 30.4 |
| LAK cells IL-2 | 41.2 |
| LAK cells IL-2 + IL-12 | 8.5 |
| LAK cells IL-2 + IFN gamma | 44.8 |
| LAK cells IL-2 + IL-18 | 40.9 |
| LAK cells PMA/ionomycin | 0.0 |
| NK Cells IL-2 rest | 27.0 |
| Two Way MLR 3 day | 34.2 |
| Two Way MLR 5 day | 9.3 |
| Two Way MLR 7 day | 33.0 |
| PBMC rest | 0.0 |
| PBMC PWM | 62.4 |
| PBMC PHA-L | 54.0 |
| Ramos (B cell) none | 0.0 |
| Ramos (B cell) ionomycin | 17.6 |
| B lymphocytes PWM | 11.5 |
| B lymphocytes CD40L and IL-4 | 10.6 |
| EOL-1 dbcAMP | 6.2 |
| EOL-1 dbcAMP PMA/ionomycin | 0.0 |
| Dendritic cells none | 8.4 |
| Dendritic cells LPS | 11.1 |
| Dendritic cells anti-CD40 | 7.6 |
| Monocytes rest | 0.0 |
| Monocytes LPS | 9.3 |
| Macrophages rest | 9.3 |
| Macrophages LPS | 0.0 |
| HUVEC none | 0.0 |
| HUVEC starved | 10.2 |
| HUVEC IL-1beta | 19.1 |
| HUVEC IFN gamma | 0.0 |
| HUVEC TNF alpha + IFN gamma | 6.8 |
| HUVEC TNF alpha + IL4 | 0.0 |
| HUVEC IL-11 | 0.0 |
| Lung Microvascular EC none | 19.3 |
| Lung Microvascular EC TNFalpha + IL-1beta | 31.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 18.2 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 89.5 |
| KU-812 (Basophil) PMA/ionomycin | 95.3 |
| CCD1106 (Keratinocytes) none | 5.8 |
| CCD1106 (Keratinocytes) TNFalpha + IL-1beta | 0.0 |
| Liver cirrhosis | 35.6 |
| Lupus kidney | 0.0 |
| NCI-H292 none | 8.9 |
| NCI-H292 IL-4 | 22.5 |
| NCI-H292 IL-9 | 39.0 |
| NCI-H292 IL-13 | 0.0 |
| NCI-H292 IFN gamma | 16.6 |
| HPAEC none | 0.0 |
| HPAEC TNF alpha + IL-1 beta | 0.0 |
| Lung fibroblast none | 21.2 |
| Lung fibroblast TNF alpha + IL-1 beta | 27.5 |
| Lung fibroblast IL-4 | 0.0 |
| Lung fibroblast IL-9 | 11.4 |
| Lung fibroblast IL-13 | 0.0 |
| Lung fibroblast IFN gamma | 20.0 |
| Dermal fibroblast CCD1070 rest | 4.2 |
| Dermal fibroblast CCD1070 TNF alpha | 41.2 |
| Dermal fibroblast CCD1070 IL-1 beta | 11.3 |
| Dermal fibroblast IFN gamma | 0.0 |
| Dermal fibroblast IL-4 | 0.0 |
| IBD Colitis 2 | 10.1 |
| IBD Crohn's | 0.0 |
| Colon | 33.0 |
| Lung | 0.0 |
| Thymus | 28.7 |
| Kidney | 100.0 |

Column A - Rel. Exp. (%) Ag436, Run 153954658

Panel 1 Summary: Ag436 Expression of this gene was highest in a breast cancer cell line, showing that this gene plays a role in the development and/or progression of breast cancer. This gene was also expressed at fairly high levels in pancreas, and testis. Expression in pancreas demonstrates that this gene or gene product is involved in metabolic diseases such as diabetes and obesity.

Panel 1.1 Summary: Ag436b The expression of this gene was highest in testes and one breast cancer cell line. Expression of this gene is useful as a unique and specific marker for normal testis tissue. Therapeutic modulation of this gene or gene product through the use of protein, antibody, or small molecule therapeutics is useful as a therapeutic for breast cancer.

Panel 1.3D Summary: Ag436 The expression of this gene was highest in testis and one breast cancer cell line.

Panel 2D Summary: Ag436 The expression of this gene in panel 2D was restricted to one bladder cancer, one kidney cancer and one breast cancer. The expression detected in breast cancer was also in agreement with the results observed for panel 1.3D.

Thus, therapeutic modulation of this gene or its protein product is useful in the treatment breast, bladder or kidney cancer.

Panel 3D Summary: Ag436 Expression of this gene in panel 3D was restricted to one sample of a plasmacytoma cell line and two lung cancer cell lines. Thus, therapeutic modulation of this gene or gene product is useful in the treatment of plasmacytoma or lung cancer.

Panel 4D Summary: Ag436 This gene was expressed in the kidney and in the KU-812 basophil cell line. Basophils are found in the kidney and may give rise to the signal observed in this tissue (Maekawa, K. FEBS Lett 1994 January 10;337(2):200-6). Therefore, therapeutics targeting this gene or its protein product are useful for the detection of basophils in a variety of tissues.

U. CG52988-04 and CG52988-06 and CG52988-07: GAGE

Expression of full-length physical clones CG52988-04, CG52988-06, and CG52988-07 was assessed using the primer-probe sets Ag437 and Ag7222, described in Tables UA and UB. Results of the RTQ-PCR runs are shown in Tables UC and UD.

TABLE UA
Probe Name Ag437

| | | | | |
|---|---|---|---|---|
| Primers | Sequences | Length | Start Position | SEQ ID No |

| | | | | |
|---|---|---|---|---|
| Forward | 5′-tcttcccagccagttggatct-3′ | 21 | 260 | 1345 |
| Probe | TET-5′-tcctcagtgggctcctggacaatca-3′-TAMRA | 25 | 234 | 1346 |
| Reverse | 5′-cagttggtggttcctcttcttga-3′ | 23 | 205 | 1347 |

TABLE UB
Probe Name Ag7222

| | | | | |
|---|---|---|---|---|
| Primers | Sequences | Length | Start Position | SEQ ID No |

| | | | | |
|---|---|---|---|---|
| Forward | 5′-ttgcacctagtggggagatc-3′ | 20 | 173 | 1348 |
| Probe | TET-5′-acctgccgttcaagggcctgac-3′-TAMRA | 22 | 137 | 1349 |
| Reverse | 5′-cagagccagttcctgttgaa-3′ | 20 | 107 | 1350 |

TABLE UC
General_screening_panel_v1.7

| Tissue Name | A |
|---|---|
| Adipose | 1.4 |
| HUVEC | 0.3 |
| Melanoma* Hs688(A).T | 0.0 |
| Melanoma* Hs688(B).T | 0.8 |
| Melanoma (met) SK-MEL-5 | 0.0 |
| Testis | 98.6 |
| Prostate ca. (bone met) PC-3 | 0.0 |
| Prostate ca. DU145 | 0.3 |
| Prostate pool | 0.0 |
| Uterus pool | 0.0 |
| Ovarian ca. OVCAR-3 | 0.3 |
| Ovarian ca. (ascites) SK-OV-3 | 0.0 |
| Ovarian ca. OVCAR-4 | 0.2 |
| Ovarian ca. OVCAR-5 | 1.5 |
| Ovarian ca. IGROV-1 | 0.7 |
| Ovarian ca. OVCAR-8 | 2.1 |
| Ovary | 0.9 |
| Breast ca. MCF-7 | 0.0 |
| Breast ca. MDA-MB-231 | 6.4 |
| Breast ca. BT-549 | 0.3 |
| Breast ca. T47D | 0.1 |
| Breast pool | 0.0 |
| Trachea | 2.6 |
| Lung | 1.6 |
| Fetal Lung | 2.5 |
| Lung ca. NCI-N417 | 0.0 |
| Lung ca. LX-1 | 0.0 |
| Lung ca. NCI-H146 | 100.0 |
| Lung ca. SHP-77 | 0.7 |
| Lung ca. NCI-H23 | 2.4 |
| Lung ca. NCI-H460 | 1.2 |
| Lung ca. HOP-62 | 2.0 |
| Lung ca. NCI-H522 | 0.6 |
| Lung ca. DMS-114 | 0.0 |
| Liver | 0.0 |
| Fetal Liver | 5.1 |
| Kidney pool | 1.1 |
| Fetal Kidney | 0.9 |
| Renal ca. 786-0 | 0.5 |
| Renal ca. A498 | 0.5 |
| Renal ca. ACHN | 0.6 |
| Renal ca. UO-31 | 0.0 |
| Renal ca. TK-10 | 0.3 |
| Bladder | 1.9 |
| Gastric ca. (liver met.) NCI-N87 | 0.0 |
| Stomach | 0.0 |
| Colon ca. SW-948 | 0.3 |
| Colon ca. SW480 | 0.6 |
| Colon ca. (SW480 met) SW620 | 2.4 |
| Colon ca. HT29 | 0.0 |
| Colon ca. HCT-116 | 2.1 |
| Colon cancer tissue | 0.0 |
| Colon ca. SW1116 | 0.8 |
| Colon ca. Colo-205 | 0.6 |
| Colon ca. SW-48 | 0.0 |
| Colon | 0.9 |
| Small Intestine | 0.3 |
| Fetal Heart | 0.3 |
| Heart | 0.0 |
| Lymph Node pool 1 | 0.0 |
| Lymph Node pool 2 | 2.9 |
| Fetal Skeletal Muscle | 1.1 |
| Skeletal Muscle pool | 0.2 |
| Skeletal Muscle | 0.3 |
| Spleen | 1.6 |
| Thymus | 0.9 |
| CNS cancer (glio/astro) SF-268 | 0.0 |
| CNS cancer (glio/astro) T98G | 0.0 |
| CNS cancer (neuro; met) SK-N-AS | 1.3 |
| CNS cancer (astro) SF-539 | 0.0 |
| CNS cancer (astro) SNB-75 | 0.3 |
| CNS cancer (glio) SNB-19 | 0.9 |
| CNS cancer (glio) SF-295 | 6.8 |
| Brain (Amygdala) | 0.3 |
| Brain (Cerebellum) | 1.2 |
| Brain (Fetal) | 1.0 |
| Brain (Hippocampus) | 0.3 |
| Cerebral Cortex pool | 0.5 |
| Brain (Substantia nigra) | 0.0 |
| Brain (Thalamus) | 0.2 |
| Brain (Whole) | 2.1 |
| Spinal Cord | 0.4 |
| Adrenal Gland | 0.4 |
| Pituitary Gland | 0.2 |
| Salivary Gland | 0.0 |
| Thyroid | 0.9 |
| Pancreatic ca. PANC-1 | 0.0 |
| Pancreas pool | 0.4 |

Column A - Rel. Exp. (%) Ag7222, Run 318040952

TABLE UD
Panel 4D

| Tissue Name | A |
|---|---|
| Secondary Th1 act | 21.9 |
| Secondary Th2 act | 23.5 |
| Secondary Tr1 act | 17.3 |
| Secondary Th1 rest | 13.6 |
| Secondary Th2 rest | 7.7 |
| Secondary Tr1 rest | 13.6 |
| Primary Th1 act | 33.2 |
| Primary Th2 act | 19.5 |
| Primary Tr1 act | 15.7 |
| Primary Th1 rest | 43.5 |
| Primary Th2 rest | 22.7 |
| Primary Tr1 rest | 19.9 |
| CD45RA CD4 lymphocyte act | 18.8 |
| CD45RO CD4 lymphocyte act | 48.6 |
| CD8 lymphocyte act | 69.3 |
| Secondary CD8 lymphocyte rest | 30.6 |
| Secondary CD8 lymphocyte act | 42.9 |
| CD4 lymphocyte none | 30.4 |
| 2ry Th1/Th2/Tr1_anti-CD95 CH11 | 12.7 |
| LAK cells rest | 9.6 |
| LAK cells IL-2 | 20.4 |
| LAK cells IL-2 + IL-12 | 0.8 |
| LAK cells IL-2 + IFN gamma | 42.3 |
| LAK cells IL-2 + IL-18 | 18.2 |
| LAK cells PMA/ionomycin | 19.5 |
| NK Cells IL-2 rest | 17.2 |
| Two Way MLR 3 day | 26.2 |
| Two Way MLR 5 day | 11.3 |
| Two Way MLR 7 day | 5.5 |
| PBMC rest | 18.0 |
| PBMC PWM | 41.5 |
| PBMC PHA-L | 24.3 |
| Ramos (B cell) none | 0.0 |
| Ramos (B cell) ionomycin | 10.2 |
| B lymphocytes PWM | 81.2 |
| B lymphocytes CD40L and IL-4 | 20.3 |
| EOL-1 dbcAMP | 0.0 |
| EOL-1 dbcAMP PMA/ionomycin | 0.0 |
| Dendritic cells none | 10.4 |
| Dendritic cells LPS | 6.3 |
| Dendritic cells anti-CD40 | 0.0 |
| Monocytes rest | 8.7 |
| Monocytes LPS | 4.4 |
| Macrophages rest | 5.4 |
| Macrophages LPS | 11.3 |
| HUVEC none | 0.0 |
| HUVEC starved | 10.4 |
| HUVEC IL-1beta | 5.1 |
| HUVEC IFN gamma | 6.3 |
| HUVEC TNF alpha + IFN gamma | 14.9 |
| HUVEC TNF alpha + IL4 | 0.0 |
| HUVEC IL-11 | 0.0 |
| Lung Microvascular EC none | 4.8 |
| Lung Microvascular EC TNFalpha + IL-1beta | 0.0 |
| Microvascular Dermal EC none | 9.5 |
| Microsvasular Dermal EC TNFalpha + IL-1beta | 0.0 |
| Bronchial epithelium TNFalpha + IL1beta | 31.0 |
| Small airway epithelium none | 12.2 |
| Small airway epithelium TNFalpha + IL-1beta | 12.3 |
| Coronery artery SMC rest | 7.0 |
| Coronery artery SMC TNFalpha + IL-1beta | 25.2 |
| Astrocytes rest | 0.0 |
| Astrocytes TNFalpha + IL-1beta | 14.8 |
| KU-812 (Basophil) rest | 62.0 |
| KU-812 (Basophil) PMA/ionomycin | 100.0 |
| CCD1106 (Keratinocytes) none | 6.0 |
| CCD1106 (Keratinocytes) TNFalpha + IL-1beta | 20.2 |
| Liver cirrhosis | 20.7 |
| Lupus kidney | 15.7 |
| NCI-H292 none | 26.1 |
| NCI-H292 IL-4 | 12.2 |
| NCI-H292 IL-9 | 6.1 |
| NCI-H292 IL-13 | 19.6 |
| NCI-H292 IFN gamma | 15.8 |
| HPAEC none | 5.4 |
| HPAEC TNF alpha + IL-1 beta | 0.0 |
| Lung fibroblast none | 0.0 |
| Lung fibroblast TNF alpha + IL-1 beta | 12.4 |
| Lung fibroblast IL-4 | 17.6 |
| Lung fibroblast IL-9 | 23.2 |
| Lung fibroblast IL-13 | 14.5 |
| Lung fibroblast IFN gamma | 12.9 |
| Dermal fibroblast CCD1070 rest | 7.0 |
| Dermal fibroblast CCD1070 TNF alpha | 26.4 |
| Dermal fibroblast CCD1070 IL-1 beta | 2.3 |
| Dermal fibroblast IFN gamma | 0.0 |
| Dermal fibroblast IL-4 | 11.0 |
| IBD Colitis 2 | 12.2 |
| IBD Crohn's | 16.4 |
| Colon | 18.7 |
| Lung | 12.2 |
| Thymus | 12.3 |
| Kidney | 37.6 |

Column A - Rel. Exp. (%) Ag437, Run 145778415

General_screening₁₃ panel_v1.7 Summary: Ag7222 Highest expression of this gene was seen in the testis and a lung cancer cell line sample (CTs=27). Thus, therapeutics targeting this gene or its protein product are useful in the treatment of male infertility or lung cancer.

Panel 4D Summary: Ag437 Highest expression was seen in the basophil cell line, KU-812 (CTs=33). Basophils release histamines and other biological modifiers in reponse to allergens and play an important role in the pathology of asthma and hypersensitivity reactions. Therefore, therapeutics designed against the protein encoded by this gene are useful for the reduction or inhibition of inflammation by blocking basophil function in these diseases. In addition, these cells are a reasonable model for the inflammatory cells that take part in various inflammatory lung and bowel diseases, such as asthma, Crohn's disease, and ulcerative colitis. Therefore, therapeutics that modulate the function of this gene or gene product are useful for the reduction or elimination of the symptoms of patients suffering from asthma, Crohn's disease, and ulcerative colitis.

V. CG5344903: Butyrophilin Like Receptor

Expression of gene CG53449-03 was assessed using the primer-probe sets Ag2030 and Ag8336, described in Tables VA and VB. Results of the RTQ-PCR runs are shown in Tables VC and VD.

TABLE VA
Probe Name Ag2030

| | | | | SEQ |
|---|---|---|---|---|
| | | | Start | ID |
| Primers | Sequences | Length | Position | No |
| Forward | 5′-ctgcgtttctgatctgaaaact-3′ | 22 | 997 | 1351 |
| Probe | TET-5′-acccatagaaaagctccccaggaggt-3′- | 26 | 1022 | 1352 |
| | TAMRA | | | |
| Reverse | 5′-ccaccacactcttccttgtaaa-3′ | 22 | 1067 | 1353 |

TABLE VB
Probe Name Ag8336

| | | | | SEQ |
|---|---|---|---|---|
| | | | Start | ID |
| Primers | Sequences | Length | Position | No |
| Forward | 5′-cattgttggactgaagattttctt-3′ | 24 | 844 | 1354 |
| Probe | TET-5′-ctccaaattccagtggaaaatccagg-3′- | 26 | 868 | 1355 |
| | TAMRA | | | |
| Reverse | 5′-cctgtccgtgctttcttctc-3′ | 20 | 907 | 1356 |

TABLE VC
Panel 1.3D

| Tissue Name | A |
|---|---|
| Liver adenocarcinoma | 0.0 |
| Pancreas | 3.3 |
| Pancreatic ca. CAPAN 2 | 0.0 |
| Adrenal gland | 0.0 |
| Thyroid | 3.8 |
| Salivary gland | 0.3 |
| Pituitary gland | 0.0 |
| Brain (fetal) | 0.0 |
| Brain (whole) | 0.0 |
| Brain (amygdala) | 0.3 |
| Brain (cerebellum) | 0.0 |
| Brain (hippocampus) | 0.0 |
| Brain (substantia nigra) | 0.0 |
| Brain (thalamus) | 0.3 |
| Cerebral Cortex | 0.0 |
| Spinal cord | 3.2 |
| glio/astro U87-MG | 0.0 |
| glio/astro U-118-MG | 0.0 |
| astrocytoma SW1783 | 0.0 |
| neuro*; met SK-N-AS | 0.0 |
| astrocytoma SF-539 | 0.0 |
| astrocytoma SNB-75 | 0.0 |
| glioma SNB-19 | 0.0 |
| glioma U251 | 0.0 |
| glioma SF-295 | 0.9 |
| Heart (fetal) | 0.0 |
| Heart | 0.0 |
| Skeletal muscle (fetal) | 0.0 |
| Skeletal muscle | 0.0 |
| Bone marrow | 30.6 |
| Thymus | 0.0 |
| Spleen | 3.0 |
| Lymph node | 4.3 |
| Colorectal | 73.7 |
| Stomach | 15.7 |
| Small intestine | 100.0 |
| Colon ca. SW480 | 0.0 |
| Colon ca.* SW620(SW480 met) | 0.0 |
| Colon ca. HT29 | 0.0 |
| Colon ca. HCT-116 | 0.0 |
| Colon ca. CaCo-2 | 40.1 |
| Colon ca. tissue(ODO3866) | 1.9 |
| Colon ca. HCC-2998 | 8.1 |
| Gastric ca.* (liver met) NCI-N87 | 0.0 |
| Bladder | 10.7 |
| Trachea | 1.6 |
| Kidney | 0.0 |
| Kidney (fetal) | 6.5 |
| Renal ca. 786-0 | 0.0 |
| Renal ca. A498 | 0.0 |
| Renal ca. RXF 393 | 0.0 |
| Renal ca. ACHN | 0.0 |
| Renal ca. UO-31 | 0.0 |
| Renal ca. TK-10 | 0.0 |
| Liver | 0.0 |
| Liver (fetal) | 1.6 |
| Liver ca. (hepatoblast) HepG2 | 0.0 |
| Lung | 27.9 |
| Lung (fetal) | 12.4 |
| Lung ca. (small cell) LX-1 | 0.4 |
| Lung ca. (small cell) NCI-H69 | 0.0 |
| Lung ca. (s. cell var.) SHP-77 | 0.0 |
| Lung ca. (large cell)NCI-H460 | 7.6 |
| Lung ca. (non-sm. cell) A549 | 1.2 |
| Lung ca. (non-s. cell) NCI-H23 | 0.0 |
| Lung ca. (non-s. cell) HOP-62 | 0.0 |
| Lung ca. (non-s. cl) NCI-H522 | 0.0 |
| Lung ca. (squam.) SW 900 | 0.0 |
| Lung ca. (squam.) NCI-H596 | 0.0 |
| Mammary gland | 0.0 |
| Breast ca.* (pl. ef) MCF-7 | 0.0 |
| Breast ca.* (pl. ef) MDA-MB-231 | 0.0 |
| Breast ca.* (pl. ef) T47D | 0.0 |
| Breast ca. BT-549 | 0.0 |
| Breast ca. MDA-N | 0.0 |
| Ovary | 0.0 |
| Ovarian ca. OVCAR-3 | 0.0 |
| Ovarian ca. OVCAR-4 | 0.0 |
| Ovarian ca. OVCAR-5 | 1.0 |
| Ovarian ca. OVCAR-8 | 0.0 |
| Ovarian ca. IGROV-1 | 0.0 |
| Ovarian ca.* (ascites) SK-OV-3 | 0.0 |
| Uterus | 0.8 |
| Placenta | 0.0 |
| Prostate | 0.0 |
| Prostate ca.* (bone met)PC-3 | 0.0 |
| Testis | 15.8 |
| Melanoma Hs688(A).T | 0.0 |
| Melanoma* (met) Hs688(B).T | 0.0 |
| Melanoma UACC-62 | 0.0 |
| Melanoma M14 | 0.0 |
| Melanoma LOX IMVI | 0.0 |
| Melanoma* (met) SK-MEL-5 | 0.0 |
| Adipose | 1.8 |

Column A - Rel. Exp. (%) Ag2030, Run 165618974

TABLE VD
Panel 4.1D

| Tissue Name | A | B |
|---|---|---|
| Secondary Th1 act | 2.3 | 0.0 |
| Secondary Th2 act | 0.8 | 0.0 |
| Secondary Tr1 act | 0.0 | 0.0 |
| Secondary Th1 rest | 0.0 | 0.0 |
| Secondary Th2 rest | 0.0 | 0.0 |
| Secondary Tr1 rest | 0.0 | 0.0 |
| Primary Th1 act | 0.0 | 0.0 |
| Primary Th2 act | 0.0 | 0.0 |
| Primary Tr1 act | 1.7 | 0.0 |
| Primary Th1 rest | 0.0 | 0.0 |
| Primary Th2 rest | 0.0 | 0.0 |
| Primary Tr1 rest | 0.0 | 0.0 |
| CD45RA CD4 lymphocyte act | 0.0 | 0.0 |
| CD45RO CD4 lymphocyte act | 2.3 | 3.6 |
| CD8 lymphocyte act | 1.2 | 0.0 |
| Secondary CD8 lymphocyte rest | 0.0 | 0.0 |
| Secondary CD8 lymphocyte act | 1.4 | 0.0 |
| CD4 lymphocyte none | 0.0 | 0.0 |
| 2ry Th1/Th2/Tr1_anti-CD95 CH11 | 0.0 | 0.0 |
| LAK cells rest | 0.0 | 0.0 |
| LAK cells IL-2 | 0.0 | 0.0 |
| LAK cells IL-2 + IL-12 | 0.0 | 0.0 |
| LAK cells IL-2 + IFN gamma | 0.0 | 0.0 |
| LAK cells IL-2 + IL-18 | 0.0 | 0.0 |
| LAK cells PMA/ionomycin | 0.0 | 0.0 |
| NK Cells IL-2 rest | 0.0 | 0.0 |
| Two Way MLR 3 day | 0.0 | 0.0 |
| Two Way MLR 5 day | 0.0 | 0.0 |
| Two Way MLR 7 day | 0.0 | 0.0 |
| PBMC rest | 0.0 | 0.0 |
| PBMC PWM | 1.1 | 0.0 |
| PBMC PHA-L | 0.0 | 0.0 |
| Ramos (B cell) none | 0.0 | 0.0 |
| Ramos (B cell) ionomycin | 0.0 | 0.0 |
| B lymphocytes PWM | 4.7 | 0.0 |
| B lymphocytes CD40L and IL-4 | 0.0 | 0.0 |
| EOL-1 dbcAMP | 0.0 | 0.0 |
| EOL-1 dbcAMP PMA/ionomycin | 0.0 | 0.0 |
| Dendritic cells none | 0.0 | 0.0 |
| Dendritic cells LPS | 0.0 | 0.0 |
| Dendritic cells anti-CD40 | 0.0 | 0.0 |
| Monocytes rest | 0.0 | 0.0 |
| Monocytes LPS | 0.0 | 0.0 |
| Macrophages rest | 0.0 | 0.0 |
| Macrophages LPS | 0.0 | 0.0 |
| HUVEC none | 0.0 | 0.0 |
| HUVEC starved | 0.0 | 0.0 |
| HUVEC IL-1beta | 0.0 | 0.0 |
| HUVEC IFN gamma | 0.0 | 0.0 |
| HUVEC TNF alpha + IFN gamma | 0.0 | 0.0 |
| HUVEC TNF alpha + IL4 | 0.0 | 0.0 |
| HUVEC IL-11 | 0.0 | 0.0 |
| Lung Microvascular EC none | 0.0 | 0.0 |
| Lung Microvascular EC TNFalpha + IL-1beta | 0.0 | 0.0 |
| Microvascular Dermal EC none | 0.0 | 0.0 |
| Microsvasular Dermal EC TNFalpha + IL-1beta | 0.0 | 0.0 |
| Bronchial epithelium TNFalpha + IL1beta | 0.0 | 0.0 |
| Small airway epithelium none | 0.0 | 0.0 |
| Small airway epithelium TNFalpha + IL-1beta | 0.0 | 0.0 |
| Coronery artery SMC rest | 0.0 | 0.0 |
| Coronery artery SMC TNFalpha + IL-1beta | 0.0 | 0.0 |
| Astrocytes rest | 0.0 | 0.0 |
| Astrocytes TNFalpha + IL-1beta | 0.0 | 0.0 |
| KU-812 (Basophil) rest | 0.0 | 0.0 |
| KU-812 (Basophil) PMA/ionomycin | 0.0 | 0.0 |
| CCD1106 (Keratinocytes) none | 0.0 | 0.0 |
| CCD1106 (Keratinocytes) TNFalpha + IL-1beta | 0.0 | 0.0 |
| Liver cirrhosis | 12.9 | 1.3 |
| NCI-H292 none | 0.0 | 3.7 |
| NCI-H292 IL-4 | 0.0 | 0.0 |
| NCI-H292 IL-9 | 0.0 | 0.0 |
| NCI-H292 IL-13 | 0.0 | 0.0 |
| NCI-H292 IFN gamma | 0.0 | 0.0 |
| HPAEC none | 0.0 | 0.0 |
| HPAEC TNF alpha + IL-1 beta | 0.0 | 0.0 |
| Lung fibroblast none | 0.0 | 0.0 |
| Lung fibroblast TNF alpha + IL-1 beta | 0.0 | 0.0 |
| Lung fibroblast IL-4 | 0.0 | 0.0 |
| Lung fibroblast IL-9 | 0.0 | 0.0 |
| Lung fibroblast IL-13 | 0.0 | 0.0 |
| Lung fibroblast IFN gamma | 0.0 | 0.0 |
| Dermal fibroblast CCD1070 rest | 0.0 | 0.0 |
| Dermal fibroblast CCD1070 TNF alpha | 0.7 | 0.0 |
| Dermal fibroblast CCD1070 IL-1 beta | 0.0 | 0.0 |
| Dermal fibroblast IFN gamma | 0.0 | 0.0 |
| Dermal fibroblast IL-4 | 0.0 | 0.0 |
| Dermal Fibroblasts rest | 0.0 | 0.0 |
| Neutrophils TNFa + LPS | 23.2 | 11.6 |
| Neutrophils rest | 84.1 | 100.0 |
| Colon | 100.0 | 5.7 |
| Lung | 2.4 | 0.0 |
| Thymus | 0.0 | 0.0 |
| Kidney | 0.8 | 0.0 |

Column A - Rel. Exp. (%) Ag2030, Run 306413257
Column B - Rel. Exp. (%) Ag8336, Run 343540037

Panel 1.3D Summary: Ag2030 This gene was most prominently expressed in gastrointestinal tissues. Expression of this gene was highest in colon and small intestine (CT=31) and was also significantly expressed in stomach tissue. Therapeutic modulation of this gene or its protein product is useful as a marker of gastrointestinal tissue, and specifically of small intestine, stomach or colorectal tissue.

Panel 4.1D Summary: Ag2030/Ag8336 Expression of this gene was limited to colon and neutrophils (CTs=30-34). Expression of this gene was down-regulated in activated neutrophils. This expression profile demonstrated that the encoded protein reduces activation of these inflammatory cells and is useful as a protein therapeutic to eliminate the symptoms in patients with Crohn's disease, ulcerative colitis, multiple sclerosis, chronic obstructive pulmonary disease, asthma, emphysema, rheumatoid arthritis, lupus erythematosus, or psoriasis. In addition, small molecule or antibodies targeting this gene or gene product are effective in increasing the immune response in patients with AIDS or other immunodeficiencies.

W. CG53449-04: B7/Butyrophilin Like Receptor

Expression of gene CG53449-04 was assessed using the primer-probe sets Ag2030, Ag6640 and Ag7598, described in Tables WA, WB and WC. Results of the RTQ-PCR runs are shown in Tables WD, WE, WF, WG, WH and WI.

TABLE WA
Probe Name Ag2030

| | | | | SEQ |
|---|---|---|---|---|
| | | | Start | ID |
| Primers | Sequences | Length | Position | No |
| Forward | 5′-ctgcgtttctgatctgaaaact-3′ | 22 | 1185 | 1357 |
| Probe | TET-5′-acccatagaaaagctccccaggaggt-3′- | 26 | 1210 | 1358 |
| | TAMRA | | | |
| Reverse | 5′-ccaccacactcttccttgtaaa-3′ | 22 | 1255 | 1359 |

TABLE WB
Probe Name Ag6640

| | | | | SEQ |
|---|---|---|---|---|
| | | | Start | ID |
| Primers | Sequences | Length | Position | No |
| Forward | 5′-gtgtaagcgagagagagaagcat-3′ | 23 | 938 | 1360 |
| Probe | TET-5′-atctctgatcctgtccctgctggaac-3′- | 26 | 981 | 1361 |
| | TAMRA | | | |
| Reverse | 5′-tagaagaagagaagcagctggat-3′ | 23 | 1015 | 1362 |

TABLE WC
Probe Name Ag7598

| | | | | SEQ |
|---|---|---|---|---|
| | | | Start | ID |
| Primers | Sequences | Length | Position | No |
| Forward | 5′-tgctcccacatccagct-3′ | 17 | 1006 | 1363 |
| Probe | TET-5′-ttcttctagtcctagcctccagggg-3′- | 25 | 1030 | 1364 |
| | TAMRA | | | |
| Reverse | 5′-gctttcttctccagtccagttc-3′ | 22 | 1084 | 1365 |

TABLE WD
AI_comprehensive panel_v1.0

| Tissue Name | A |
|---|---|
| 110967 COPD-F | 13.5 |
| 110980 COPD-F | 25.5 |
| 110968 COPD-M | 15.1 |
| 110977 COPD-M | 42.0 |
| 110989 Emphysema-F | 6.3 |
| 110992 Emphysema-F | 0.0 |
| 110993 Emphysema-F | 6.7 |
| 110994 Emphysema-F | 2.0 |
| 110995 Emphysema-F | 1.0 |
| 110996 Emphysema-F | 0.0 |
| 110997 Asthma-M | 1.6 |
| 111001 Asthma-F | 6.0 |
| 111002 Asthma-F | 4.0 |
| 111003 Atopic Asthma-F | 3.5 |
| 111004 Atopic Asthma-F | 3.4 |
| 111005 Atopic Asthma-F | 4.5 |
| 111006 Atopic Asthma-F | 0.0 |
| 111417 Allergy-M | 4.5 |
| 112347 Allergy-M | 0.4 |
| 112349 Normal Lung-F | 0.4 |
| 112357 Normal Lung-F | 8.9 |
| 112354 Normal Lung-M | 4.8 |
| 112374 Crohns-F | 4.2 |
| 112389 Match Control Crohns-F | 7.2 |
| 112375 Crohns-F | 1.9 |
| 112732 Match Control Crohns-F | 3.9 |
| 112725 Crohns-M | 3.5 |
| 112387 Match Control Crohns-M | 0.3 |
| 112378 Crohns-M | 1.2 |
| 112390 Match Control Crohns-M | 24.5 |
| 112726 Crohns-M | 2.0 |
| 112731 Match Control Crohns-M | 4.5 |
| 112380 Ulcer Col-F | 7.2 |
| 112734 Match Control Ulcer Col-F | 15.7 |
| 112384 Ulcer Col-F | 13.1 |
| 112737 Match Control Ulcer Col-F | 3.1 |
| 112386 Ulcer Col-F | 5.6 |
| 112738 Match Control Ulcer Col-F | 19.9 |
| 112381 Ulcer Col-M | 0.3 |
| 112735 Match Control Ulcer Col-M | 0.0 |
| 112382 Ulcer Col-M | 18.7 |
| 112394 Match Control Ulcer Col-M | 3.4 |
| 112383 Ulcer Col-M | 2.6 |
| 112736 Match Control Ulcer Col-M | 6.2 |
| 112423 Psoriasis-F | 12.3 |
| 112427 Match Control Psoriasis-F | 25.2 |
| 112418 Psoriasis-M | 26.8 |
| 112723 Match Control Psoriasis-M | 2.9 |
| 112419 Psoriasis-M | 18.3 |
| 112424 Match Control Psoriasis-M | 11.7 |
| 112420 Psoriasis-M | 32.8 |
| 112425 Match Control Psoriasis-M | 14.2 |
| 104689 (MF) OA Bone-Backus | 24.5 |
| 104690 (MF) Adj “Normal” Bone-Backus | 38.4 |
| 104691 (MF) OA Synovium-Backus | 6.4 |
| 104692 (BA) OA Cartilage-Backus | 0.0 |
| 104694 (BA) OA Bone-Backus | 6.0 |
| 104695 (BA) Adj “Normal” Bone-Backus | 37.4 |
| 104696 (BA) OA Synovium-Backus | 3.4 |
| 104700 (SS) OA Bone-Backus | 88.3 |
| 104701 (SS) Adj “Normal” Bone-Backus | 30.4 |
| 104702 (SS) OA Synovium-Backus | 28.3 |
| 117093 OA Cartilage Rep7 | 15.2 |
| 112672 OA Bone5 | 13.4 |
| 112673 OA Synovium5 | 7.5 |
| 112674 OA Synovial Fluid cells5 | 6.0 |
| 117100 OA Cartilage Rep14 | 4.5 |
| 112756 OA Bone9 | 14.2 |
| 112757 OA Synovium9 | 24.1 |
| 112758 OA Synovial Fluid Cells9 | 37.9 |
| 117125 RA Cartilage Rep2 | 10.6 |
| 113492 Bone2 RA | 100.0 |
| 113493 Synovium2 RA | 47.0 |
| 113494 Syn Fluid Cells RA | 68.3 |
| 113499 Cartilage4 RA | 67.8 |
| 113500 Bone4 RA | 81.2 |
| 113501 Synovium4 RA | 64.2 |
| 113502 Syn Fluid Cells4 RA | 51.1 |
| 113495 Cartilage3 RA | 68.8 |
| 113496 Bone3 RA | 66.9 |
| 113497 Synovium3 RA | 52.5 |
| 113498 Syn Fluid Cells3 RA | 85.9 |
| 117106 Normal Cartilage Rep20 | 1.1 |
| 113663 Bone3 Normal | 0.0 |
| 113664 Synovium3 Normal | 0.0 |
| 113665 Syn Fluid Cells3 Normal | 0.0 |
| 117107 Normal Cartilage Rep22 | 7.9 |
| 113667 Bone4 Normal | 5.1 |
| 113668 Synovium4 Normal | 3.4 |
| 113669 Syn Fluid Cells4 Normal | 3.7 |

Column A - Rel. Exp. (%) Ag6640, Run 296559279

TABLE WE
General_screening_panel_v1.6

| Tissue Name | A |
|---|---|
| Adipose | 11.0 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 16.5 |
| Prostate ca.* (bone met) PC-3 | 0.1 |
| Prostate Pool | 1.4 |
| Placenta | 0.9 |
| Uterus Pool | 0.3 |
| Ovarian ca. OVCAR-3 | 0.2 |
| Ovarian ca. SK-OV-3 | 0.0 |
| Ovarian ca. OVCAR-4 | 0.0 |
| Ovarian ca. OVCAR-5 | 3.8 |
| Ovarian ca. IGROV-1 | 0.0 |
| Ovarian ca. OVCAR-8 | 0.0 |
| Ovary | 2.9 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 2.5 |
| Trachea | 1.2 |
| Lung | 0.1 |
| Fetal Lung | 17.8 |
| Lung ca. NCI-N417 | 0.0 |
| Lung ca. LX-1 | 14.9 |
| Lung ca. NCI-H146 | 0.0 |
| Lung ca. SHP-77 | 0.2 |
| Lung ca. A549 | 1.3 |
| Lung ca. NCI-H526 | 0.0 |
| Lung ca. NCI-H23 | 1.1 |
| Lung ca. NCI-H460 | 1.1 |
| Lung ca. HOP-62 | 0.0 |
| Lung ca. NCI-H522 | 0.0 |
| Liver | 0.0 |
| Fetal Liver | 1.3 |
| Liver ca. HepG2 | 0.0 |
| Kidney Pool | 5.6 |
| Fetal Kidney | 10.3 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 13.0 |
| Gastric ca. (liver met.) NCI-N87 | 0.3 |
| Gastric ca. KATO III | 0.6 |
| Colon ca. SW-948 | 1.4 |
| Colon ca. SW480 | 0.8 |
| Colon ca.* (SW480 met) SW620 | 0.1 |
| Colon ca. HT29 | 0.4 |
| Colon ca. HCT-116 | 0.0 |
| Colon ca. CaCo-2 | 100.0 |
| Colon cancer tissue | 9.3 |
| Colon ca. SW1116 | 0.0 |
| Colon ca. Colo-205 | 7.0 |
| Colon ca. SW-48 | 26.2 |
| Colon Pool | 1.3 |
| Small Intestine Pool | 2.3 |
| Stomach Pool | 1.6 |
| Bone Marrow Pool | 0.6 |
| Fetal Heart | 2.1 |
| Heart Pool | 2.1 |
| Lymph Node Pool | 2.1 |
| Fetal Skeletal Muscle | 4.5 |
| Skeletal Muscle Pool | 0.0 |
| Spleen Pool | 8.2 |
| Thymus Pool | 1.7 |
| CNS cancer (glio/astro) U87-MG | 0.0 |
| CNS cancer (glio/astro) U-118-MG | 8.4 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.3 |
| Brain (Amygdala) Pool | 0.0 |
| Brain (cerebellum) | 0.4 |
| Brain (fetal) | 0.3 |
| Brain (Hippocampus) Pool | 0.8 |
| Cerebral Cortex Pool | 3.8 |
| Brain (Substantia nigra) Pool | 0.0 |
| Brain (Thalamus) Pool | 0.0 |
| Brain (whole) | 1.0 |
| Spinal Cord Pool | 0.4 |
| Adrenal Gland | 1.6 |
| Pituitary gland Pool | 0.0 |
| Salivary Gland | 0.7 |
| Thyroid (female) | 0.3 |
| Pancreatic ca. CAPAN2 | 0.0 |
| Pancreas Pool | 6.0 |

Column A - Rel. Exp. (%) Ag6640, Run 277241469

TABLE WF
PGI1.0

| Tissue Name | A |
|---|---|
| 162191_Normal Lung 1 (IBS) | 4.6 |
| 162570_Normal Lung 4 (Aastrand) | 2.0 |
| 160468_MD lung | 2.9 |
| 156629_MD Lung 13 | 1.6 |
| 162571 _Normal Lung 3 (Aastrand) | 0.7 |
| 162186_Fibrosis Lung 1 (Genomic | 26.4 |
| Collaborative) | |
| 162187_Fibrosis Lung 2 (Genomic | 31.6 |
| Collaborative) | |
| 151281_Fibrosis lung 11(Ardais) | 45.1 |
| 162190_Asthma Lung 4 (Genomic | 45.7 |
| Collaborative) | |
| 160467_Asthma Lung 13 (MD) | 2.1 |
| 137027_Emphysema Lung 1 (Ardais) | 6.0 |
| 137028_Emphysema Lung 2 (Ardais) | 5.7 |
| 137040_Emphysema Lung 3 (Ardais) | 7.0 |
| 137041_Emphysema Lung 4 (Ardais) | 14.1 |
| 137043_Emphysema Lung 5 (Ardais) | 7.5 |
| 142817_Emphysema Lung 6 (Ardais) | 20.4 |
| 142818_Emphysema Lung 7 (Ardais) | 19.3 |
| 142819_Emphysema Lung 8 (Ardais) | 10.4 |
| 142820_Emphysema Lung 9 (Ardais) | 1.8 |
| 142821_Emphysema Lung 10 (Ardais) | 12.6 |
| 162185_Emphysema Lung 12 (Ardais) | 13.9 |
| 162184_Emphysema Lung 13 (Ardais) | 5.3 |
| 162183_Emphysema Lung 14 (Ardais) | 28.5 |
| 162188_Emphysema Lung 15 (Genomic | 20.2 |
| Collaborative) | |
| 162177_NAT UC Colon 1(Ardais) | 100.0 |
| 162176_UC Colon 1(Ardais) | 59.9 |
| 162179_NAT UC Colon 2(Ardais) | 65.5 |
| 162178_UC Colon 2(Ardais) | 8.1 |
| 162181_NAT UC Colon 3(Ardais) | 63.7 |
| 162180_UC Colon 3(Ardais) | 5.2 |
| 162182_NAT UC Colon 4 (Ardais) | 100.0 |
| 137042_UC Colon 1108 | 4.2 |
| 137029_UC Colon 8215 | 8.1 |
| 137031_UC Colon 8217 | 5.6 |
| 137036_UC Colon 1137 | 22.2 |
| 137038_UC Colon 1491 | 13.4 |
| 137039_UC Colon 1546 | 27.0 |
| 162594_NAT Crohn's 47751 (NDRI) | 3.7 |
| 162593_Crohn's 47751 (NDRI) | 0.5 |

Column A - Rel. Exp. (%) Ag6640, Run 376378448

TABLE WG
Panel 1.3D

| Tissue Name | A |
|---|---|
| Liver adenocarcinoma | 0.0 |
| Pancreas | 3.3 |
| Pancreatic ca. CAPAN 2 | 0.0 |
| Adrenal gland | 0.0 |
| Thyroid | 3.8 |
| Salivary gland | 0.3 |
| Pituitary gland | 0.0 |
| Brain (fetal) | 0.0 |
| Brain (whole) | 0.0 |
| Brain (amygdala) | 0.3 |
| Brain (cerebellum) | 0.0 |
| Brain (hippocampus) | 0.0 |
| Brain (substantia nigra) | 0.0 |
| Brain (thalamus) | 0.3 |
| Cerebral Cortex | 0.0 |
| Spinal cord | 3.2 |
| glio/astro U87-MG | 0.0 |
| glio/astro U-118-MG | 0.0 |
| astrocytoma SW1783 | 0.0 |
| neuro*; met SK-N-AS | 0.0 |
| astrocytoma SF-539 | 0.0 |
| astrocytoma SNB-75 | 0.0 |
| glioma SNB-19 | 0.0 |
| glioma U251 | 0.0 |
| glioma SF-295 | 0.9 |
| Heart (fetal) | 0.0 |
| Heart | 0.0 |
| Skeletal muscle (fetal) | 0.0 |
| Skeletal muscle | 0.0 |
| Bone marrow | 30.6 |
| Thymus | 0.0 |
| Spleen | 3.0 |
| Lymph node | 4.3 |
| Colorectal | 73.7 |
| Stomach | 15.7 |
| Small intestine | 100.0 |
| Colon ca. SW480 | 0.0 |
| Colon ca.* SW620(SW480 met) | 0.0 |
| Colon ca. HT29 | 0.0 |
| Colon ca. HCT-116 | 0.0 |
| Colon ca. CaCo-2 | 40.1 |
| Colon ca. tissue(ODO3866) | 1.9 |
| Colon ca. HCC-2998 | 8.1 |
| Gastric ca.* (liver met) NCI-N87 | 0.0 |
| Bladder | 10.7 |
| Trachea | 1.6 |
| Kidney | 0.0 |
| Kidney (fetal) | 6.5 |
| Renal ca. 786-0 | 0.0 |
| Renal ca. A498 | 0.0 |
| Renal ca. RXF 393 | 0.0 |
| Renal ca. ACHN | 0.0 |
| Renal ca. UO-31 | 0.0 |
| Renal ca. TK-10 | 0.0 |
| Liver | 0.0 |
| Liver (fetal) | 1.6 |
| Liver ca. (hepatoblast) HepG2 | 0.0 |
| Lung | 27.9 |
| Lung (fetal) | 12.4 |
| Lung ca. (small cell) LX-1 | 0.4 |
| Lung ca. (small cell) NCI-H69 | 0.0 |
| Lung ca. (s. cell var.) SHP-77 | 0.0 |
| Lung ca. (large cell)NCI-H460 | 7.6 |
| Lung ca. (non-sm. cell) A549 | 1.2 |
| Lung ca. (non-s. cell) NCI-H23 | 0.0 |
| Lung ca. (non-s. cell) HOP-62 | 0.0 |
| Lung ca. (non-s. cl) NCI-H522 | 0.0 |
| Lung ca. (squam.) SW 900 | 0.0 |
| Lung ca. (squam.) NCI-H596 | 0.0 |
| Mammary gland | 0.0 |
| Breast ca.* (pl. ef) MCF-7 | 0.0 |
| Breast ca.* (pl. ef) MDA-MB-231 | 0.0 |
| Breast ca.* (pl. ef) T47D | 0.0 |
| Breast ca. BT-549 | 0.0 |
| Breast ca. MDA-N | 0.0 |
| Ovary | 0.0 |
| Ovarian ca. OVCAR-3 | 0.0 |
| Ovarian ca. OVCAR-4 | 0.0 |
| Ovarian ca. OVCAR-5 | 1.0 |
| Ovarian ca. OVCAR-8 | 0.0 |
| Ovarian ca. IGROV-1 | 0.0 |
| Ovarian ca.* (ascites) SK-OV-3 | 0.0 |
| Uterus | 0.8 |
| Placenta | 0.0 |
| Prostate | 0.0 |
| Prostate ca.* (bone met)PC-3 | 0.0 |
| Testis | 15.8 |
| Melanoma Hs688(A).T | 0.0 |
| Melanoma* (met) Hs688(B).T | 0.0 |
| Melanoma UACC-62 | 0.0 |
| Melanoma M14 | 0.0 |
| Melanoma LOX IMVI | 0.0 |
| Melanoma* (met) SK-MEL-5 | 0.0 |
| Adipose | 1.8 |

Column A - Rel. Exp. (%) Ag2030, Run 165618974

TABLE WH
Panel 4.1D

| Tissue Name | A | B | C | D |
|---|---|---|---|---|
| Secondary Th1 act | 2.3 | 0.0 | 0.0 | 0.0 |
| Secondary Th2 act | 0.8 | 0.0 | 0.0 | 0.0 |
| Secondary Tr1 act | 0.0 | 0.0 | 0.0 | 0.0 |
| Secondary Th1 rest | 0.0 | 0.0 | 0.0 | 0.0 |
| Secondary Th2 rest | 0.0 | 0.0 | 0.0 | 0.0 |
| Secondary Tr1 rest | 0.0 | 0.0 | 0.0 | 0.0 |
| Primary Th1 act | 0.0 | 0.0 | 0.0 | 0.0 |
| Primary Th2 act | 0.0 | 0.0 | 0.0 | 0.0 |
| Primary Tr1 act | 1.7 | 0.0 | 0.0 | 1.6 |
| Primary Th1 rest | 0.0 | 0.0 | 0.0 | 0.0 |
| Primary Th2 rest | 0.0 | 0.0 | 0.0 | 0.0 |
| Primary Tr1 rest | 0.0 | 0.0 | 0.0 | 0.0 |
| CD45RA CD4 lymphocyte act | 0.0 | 0.9 | 0.0 | 0.0 |
| CD45RO CD4 lymphocyte act | 2.3 | 3.0 | 4.0 | 9.5 |
| CD8 lymphocyte act | 1.2 | 0.0 | 0.7 | 0.0 |
| Secondary CD8 lymphocyte rest | 0.0 | 0.0 | 0.0 | 0.0 |
| Secondary CD8 lymphocyte act | 1.4 | 0.7 | 0.0 | 0.0 |
| CD4 lymphocyte none | 0.0 | 0.0 | 0.0 | 0.0 |
| 2ry Th1/Th2/Tr1_anti-CD95 CH11 | 0.0 | 0.0 | 0.9 | 0.0 |
| LAK cells rest | 0.0 | 0.0 | 1.5 | 0.0 |
| LAK cells IL-2 | 0.0 | 0.0 | 0.0 | 0.0 |
| LAK cells IL-2 + IL-12 | 0.0 | 0.0 | 0.0 | 0.0 |
| LAK cells IL-2 + IFN gamma | 0.0 | 0.0 | 0.0 | 0.0 |
| LAK cells IL-2 + IL-18 | 0.0 | 0.0 | 0.0 | 0.0 |
| LAK cells PMA/ionomycin | 0.0 | 0.0 | 0.0 | 0.0 |
| NK Cells IL-2 rest | 0.0 | 0.0 | 0.0 | 0.0 |
| Two Way MLR 3 day | 0.0 | 0.0 | 0.0 | 0.0 |
| Two Way MLR 5 day | 0.0 | 0.0 | 1.1 | 0.0 |
| Two Way MLR 7 day | 0.0 | 1.5 | 0.0 | 0.0 |
| PBMC rest | 0.0 | 0.0 | 0.0 | 0.0 |
| PBMC PWM | 1.1 | 0.0 | 0.5 | 0.0 |
| PBMC PHA-L | 0.0 | 0.0 | 0.5 | 0.0 |
| Ramos (B cell) none | 0.0 | 0.0 | 0.0 | 0.0 |
| Ramos (B cell) ionomycin | 0.0 | 0.0 | 0.0 | 0.0 |
| B lymphocytes PWM | 4.7 | 2.8 | 2.7 | 1.5 |
| B lymphocytes CD40L and IL-4 | 0.0 | 2.3 | 0.0 | 0.0 |
| EOL-1 dbcAMP | 0.0 | 0.0 | 0.0 | 0.0 |
| EOL-1 dbcAMP PMA/ionomycin | 0.0 | 0.0 | 0.0 | 0.0 |
| Dendritic cells none | 0.0 | 0.0 | 0.0 | 0.0 |
| Dendritic cells LPS | 0.0 | 0.0 | 0.0 | 0.0 |
| Dendritic cells anti-CD40 | 0.0 | 0.0 | 0.0 | 0.0 |
| Monocytes rest | 0.0 | 0.0 | 0.3 | 0.0 |
| Monocytes LPS | 0.0 | 0.0 | 0.0 | 0.0 |
| Macrophages rest | 0.0 | 0.0 | 0.0 | 0.0 |
| Macrophages LPS | 0.0 | 0.0 | 0.0 | 0.0 |
| HUVEC none | 0.0 | 0.0 | 0.0 | 0.0 |
| HUVEC starved | 0.0 | 0.0 | 0.0 | 0.0 |
| HUVEC IL-1 beta | 0.0 | 0.0 | 0.0 | 0.0 |
| HUVEC IFN gamma | 0.0 | 0.0 | 0.0 | 0.0 |
| HUVEC TNF alpha + IFN gamma | 0.0 | 0.0 | 0.0 | 0.0 |
| HUVEC TNF alpha + IL4 | 0.0 | 0.0 | 0.0 | 0.0 |
| HUVEC IL-11 | 0.0 | 0.0 | 0.0 | 0.0 |
| Lung Microvascular EC none | 0.0 | 0.0 | 0.0 | 1.7 |
| Lung Microvascular EC | 0.0 | 0.0 | 0.0 | 0.0 |
| TNFalpha + IL-1beta | | | | |
| Microvascular Dermal EC none | 0.0 | 0.0 | 1.1 | 0.0 |
| Microsvasular Dermal EC | 0.0 | 0.0 | 0.0 | 0.0 |
| TNFalpha + IL-1beta | | | | |
| Bronchial epithelium | 0.0 | 0.0 | 0.0 | 0.0 |
| TNFalpha + IL1beta | | | | |
| Small airway epithelium none | 0.0 | 0.0 | 0.0 | 0.0 |
| Small airway epithelium | 0.0 | 0.0 | 0.0 | 0.0 |
| TNFalpha + IL-1beta | | | | |
| Coronery artery SMC rest | 0.0 | 0.0 | 0.0 | 0.0 |
| Coronery artery SMC | 0.0 | 0.0 | 0.0 | 0.0 |
| TNFalpha + IL-1beta | | | | |
| Astrocytes rest | 0.0 | 0.0 | 0.0 | 0.0 |
| Astrocytes TNFalpha + IL-1beta | 0.0 | 0.0 | 0.0 | 0.0 |
| KU-812 (Basophil) rest | 0.0 | 0.0 | 0.9 | 0.0 |
| KU-812 (Basophil) PMA/ionomycin | 0.0 | 0.0 | 0.0 | 0.0 |
| CCD1106 (Keratinocytes) none | 0.0 | 0.0 | 0.0 | 0.0 |
| CCD1106 (Keratinocytes) | 0.0 | 0.0 | 0.0 | 0.0 |
| TNFalpha + IL-1beta | | | | |
| Liver cirrhosis | 12.9 | 2.8 | 6.0 | 0.0 |
| NCI-H292 none | 0.0 | 2.4 | 0.0 | 0.0 |
| NCI-H292 IL-4 | 0.0 | 2.4 | 0.0 | 1.7 |
| NCI-H292 IL-9 | 0.0 | 1.1 | 3.7 | 1.3 |
| NCI-H292 IL-13 | 0.0 | 0.0 | 1.6 | 1.6 |
| NCI-H292 IFN gamma | 0.0 | 0.0 | 0.0 | 0.0 |
| HPAEC none | 0.0 | 0.0 | 0.0 | 0.0 |
| HPAEC TNF alpha + IL-1 beta | 0.0 | 0.0 | 0.0 | 0.0 |
| Lung fibroblast none | 0.0 | 0.0 | 0.0 | 0.0 |
| Lung fibroblast TNF alpha + IL-1 beta | 0.0 | 0.0 | 0.0 | 0.0 |
| Lung fibroblast IL-4 | 0.0 | 0.0 | 0.0 | 0.0 |
| Lung fibroblast IL-9 | 0.0 | 0.0 | 0.0 | 0.0 |
| Lung fibroblast IL-13 | 0.0 | 0.0 | 0.0 | 0.0 |
| Lung fibroblast IFN gamma | 0.0 | 0.0 | 0.0 | 0.0 |
| Dermal fibroblast CCD1070 rest | 0.0 | 0.0 | 0.0 | 0.0 |
| Dermal fibroblast CCD1070 TNF alpha | 0.7 | 0.0 | 0.0 | 0.0 |
| Dermal fibroblast CCD1070 IL-1 beta | 0.0 | 0.0 | 0.0 | 0.0 |
| Dermal fibroblast IFN gamma | 0.0 | 0.0 | 1.1 | 0.0 |
| Dermal fibroblast IL-4 | 0.0 | 0.0 | 0.0 | 0.0 |
| Dermal Fibroblasts rest | 0.0 | 0.0 | 0.0 | 0.0 |
| Neutrophils TNFa + LPS | 23.2 | 31.0 | 20.6 | 38.4 |
| Neutrophils rest | 84.1 | 100.0 | 100.0 | 100.0 |
| Colon | 100.0 | 23.8 | 25.5 | 40.3 |
| Lung | 2.4 | 0.0 | 0.7 | 0.0 |
| Thymus | 0.0 | 0.8 | 0.0 | 0.0 |
| Kidney | 0.8 | 3.0 | 1.3 | 0.0 |

Column A - Rel. Exp. (%) Ag2030, Run 306413257
Column B - Rel. Exp. (%) Ag6640, Run 274991372
Column C - Rel. Exp. (%) Ag6640, Run 296907633
Column D - Rel. Exp. (%) Ag7598, Run 310594484

TABLE WI
Panel 4D

| Tissue Name | A |
|---|---|
| Secondary Th1 act | 0.1 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.1 |
| Primary Tr1 act | 0.1 |
| Primary Th1 rest | 0.1 |
| Primary Th2 rest | 0.1 |
| Primary Tr1 rest | 0.0 |
| CD45RA CD4 lymphocyte act | 0.0 |
| CD45RO CD4 lymphocyte act | 0.0 |
| CD8 lymphocyte act | 0.1 |
| Secondary CD8 lymphocyte rest | 0.0 |
| Secondary CD8 lymphocyte act | 0.2 |
| CD4 lymphocyte none | 0.0 |
| 2ry Th1/Th2/Tr1_anti-CD95 CH11 | 0.1 |
| LAK cells rest | 0.1 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.1 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.3 |
| PBMC PHA-L | 0.1 |
| Ramos (B cell) none | 0.0 |
| Ramos (B cell) ionomycin | 0.1 |
| B lymphocytes PWM | 2.5 |
| B lymphocytes CD40L and IL-4 | 0.1 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1 beta | 0.0 |
| HUVEC IFN gamma | 0.0 |
| HUVEC TNF alpha + IFN gamma | 0.1 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.1 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL-1beta | 0.0 |
| Liver cirrhosis | 0.6 |
| Lupus kidney | 0.0 |
| NCI-H292 none | 0.1 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.1 |
| Dermal fibroblast CCD1070 IL-1 beta | 0.0 |
| Dermal fibroblast IFN gamma | 0.0 |
| Dermal fibroblast IL-4 | 0.0 |
| IBD Colitis 2 | 0.0 |
| IBD Crohn's | 5.3 |
| Colon | 100.0 |
| Lung | 0.5 |
| Thymus | 0.5 |
| Kidney | 0.0 |

Column A - Rel. Exp. (%) Ag2030, Run 161379401

AI_comprehensive panel_v1.0 Summary: Ag6640 Highest expression of this gene was detected in rheumatoid arthritis bone (CT=31). This gene was expressed at higher levels in rheumatoid arthritis (RA) tissues and osteoarthritis (OA) bone. In addition, significant expression of this gene was also seen in COPD lung, and psoriasis samples. This gene encodes a homolog of butyrophilin like receptor and is a member of the B7 family. B7 family members are costimulatory molecules, which are necessary for optimal activation of T lymphocytes after T cell receptor (TCR) engagement. The most extensively studied costimulatory molecules are B7-1 (CD80) and B7-2 (CD86). Interaction of CD28 on T cells with either B7-1 or B7-2 augments T cell activation and promotes T cell survival (Chambers, C. A. & Allison, J. P., 1997, Curr. Opin. Immunol. 9, 396-404). In contrast, binding of B7-1 or B7-2 with cytotoxic T lymphocyte antigen 4 (CTLA-4), a homolog of CD28, inhibits the T cell response by delivering a putative negative signal. Therapeutic modulation of this gene, expressed protein and/or use of antibodies or small molecule drug targeting the gene or gene product is effective in the treatment of psoriases, osteoarthritis and rheumatoid arthritis.

General_screening₁₃ panel_v1.6 Summary: Significant expression of this gene was seen in colon cancer cell lines, a lung cancer and a brain cancer cell lines. Therefore, expression of this gene is useful as a diagnostic marker and therapeutic modulation of this gene, expressed protein, and/or use of antibodies or small molecule drug targeting this gene or gene product is useful in the treatment of colon, lung and brain cancers.

Low expression of this gene was also seen in metabolic tissues including adipose, pancrease, and fetal skeletal muscle. Therefore, modulation of this gene or gene product is useful in the treatment of metabolic disorders such as diabetes and obesity.

Low expression of this gene was also seen fetal lung. Expression of this gene was higher in fetal (CTs=32-34) relative to adult lung and skeletal muscle (CTs=40). The relative overexpression of this gene in fetal tissue shows that the protein product enhances lung and skeletal muscle growth or development in the fetus and also acts in a regenerative capacity in the adult. Therapeutic modulation of this gene or the protein encoded by this gene is useful in the treatment of lung and skeletal muscle related diseases.

PGI1.0 Summary: Ag6640 Highest expression of this gene was detected in normal adjacent colon tissue from a ulcerative colitis patient (CT=26.4). Significant expression of this gene was also detected in normal adjacent and ulcerative colitis tissue samples. Expression of this gene was higher in normal relative to diseased tissue. Modulation of this gene and/or its expressed protein is useful in the treatment of ulcerative colitis.

In addition, high to moderate expression of this gene was also detected in fibrosis, asthma and emphyzema lung. Therapeutic modulation of this gene, expressed protein is useful in the treatment of fibrosis, asthma and emphysema.

Panel 1.3D Summary: Ag2030 This gene was most prominently expressed in gastrointestinal tissues. Expression of this gene was highest in colon and small intestine (CT=31) and was also significantly expressed in stomach tissue. Expression of this gene is useful as a marker of gastrointestinal tissue, and specifically of small intestine, stomach or colorectal tissue.

Panel 4.1D Summary: Ag2030/Ag6640/Ag7598 Expression of this gene was limited to colon and neutrophils (CTs=30-34). Expression of this gene was down-regulated in activated neutrophils. This expression profile demonstrates that the encoded protein reduces activation of these inflammatory cells and is useful as a protein therapeutic to eliminate the symptoms in patients with Crohn's disease, ulcerative colitis, multiple sclerosis, chronic obstructive pulmonary disease, asthma, emphysema, rheumatoid arthritis, lupus erythematosus, or psoriasis. In addition, small molecule or antibodies targeting this gene or gene product are effective in increasing the immune response in patients with AIDS or other immunodeficiencies.

Panel 4D Summary: Ag2030 The gene expression on this panel was limited essentially to normal colon. These results were consistent with what is observed in Panel 1.3D. This gene was not expressed or was expressed only at low levels in IBD colitis and Crohn's disease. Therefore, this gene is useful as a marker to distinguish normal colon from diseased colon. This gene encodes a protein with homology to the butyrophilin-like receptor. The butyrophilin-like membrane proteins are similar to the B7 family of co-stimulatory factors. B7 proteins are expressed on many cell types and function in the process of antigen presentation to T lymphocytes in the stimulation of the immune response. Recently identified B7 family members have included inhibitory proteins that reduced the activation of T lymphocytes upon cell-cell interaction. This gene or gene product is useful for the modulation of the functions associated with antigen presentation to T cells. Replacement of this gene product by gene therapy or use of the isolated extracellular domain of the gene protein is of use as a therapeutic in the treatment of IBD colitis and Crohn's disease.

X. CG53908-01: Netrin Receptor Unc5h1

Expression of full-length physical clone CG53908-01 was assessed using the primer-probe sets Ag1395 and Ag8392, described in Tables XA and XB. Results of the RTQ-PCR runs are shown in Tables XC, XD, XE, XF, XG, XH, XL XJ, and XK.

TABLE XA
Probe Name Ag1395

| | | | | SEQ |
|---|---|---|---|---|
| | | | Start | ID |
| Primers | Sequences | Length | Position | No |
| Forward | 5′-ctgcacttcaaggacagttacc-3′ | 22 | 2184 | 1366 |
| Probe | TET-5′-ctatccatccacgatgtgcccagct-3′- | 25 | 2217 | 1367 |
| | TAMRA | | | |
| Reverse | 5′-tgacaaggagcttactcttcca-3′ | 22 | 2247 | 1368 |

TABLE XB
Probe Name Ag8392

| | | | | SEQ |
|---|---|---|---|---|
| | | | Start | ID |
| Primers | Sequences | Length | Position | No |
| Forward | 5′-ccctctatgtgggcctcat-3′ | 19 | 1168 | 1369 |
| Probe | TET-5′-tgcttgtcctcatcctcgtttattgc-3′- | 26 | 1216 | 1370 |
| | TAMRA | | | |
| Reverse | 5′-tccagcccctccttcttc-3′ | 18 | 1244 | 1371 |

TABLE XC
Ardais Breast1.0

| Tissue Name | A | B |
|---|---|---|
| 97739_Breast cancer (CHTN20676) | 0.6 | 0.2 |
| 105689_Breast cancer 2B | 0.0 | 0.1 |
| 111297_Breast cancer metastasis (9369)* | 23.0 | 17.8 |
| 145848_Breast cancer (9B6) | 8.7 | 6.0 |
| 145859_Breast cancer (9EC) | 1.0 | 0.6 |
| 153632_Breast cancer (D39) | 1.0 | 0.2 |
| 153643_Breast cancer (D44) | 6.0 | 2.6 |
| 164672_Breast cancer (7464) | 9.1 | 4.5 |
| 164681_Breast cancer (5787) | 6.8 | 3.3 |
| 97748_Breast cancer (CHTN20931) | 9.3 | 0.1 |
| 105690_Breast NAT 2A | 1.7 | 0.6 |
| 111302_Breast NAT (6314) | 1.8 | 1.2 |
| 145850_Breast cancer (9C7) | 2.8 | 0.8 |
| 149844_Breast cancer (24178) | 3.3 | 2.6 |
| 153633_Breast cancer (D3A) | 72.7 | 34.4 |
| 153644_Breast cancer (D45) | 1.2 | 0.8 |
| 164673_Breast cancer (8452) | 2.9 | 2.6 |
| 164682_Breast cancer (6342)* | 2.1 | 1.5 |
| 97751_Breast cancer (CHTN21053) | 3.5 | 3.2 |
| 105694_Breast NAT | 15.8 | 10.6 |
| 116417_Breast cancer (3367)* | 1.8 | 1.5 |
| 145852_Breast cancer (A1A) | 1.0 | 0.4 |
| 151097_Breast cancer (CHTN24298) | 0.9 | 0.3 |
| 153634_Breast cancer (D3B) | 1.0 | 0.5 |
| 155797_Breast cancer (EA6) | 81.2 | 67.8 |
| 164674_Breast cancer (8811) | 2.8 | 2.4 |
| 164683_Breast cancer (6470) | 100.0 | 100.0 |
| 97763_Breast cancer (OD06083) | 3.7 | 2.4 |
| 108830_Breast cancer metastasis | 1.1 | 0.4 |
| (OD06855)* | | |
| 116418_Breast cancer (3378)* | 2.4 | 1.7 |
| 145853_Breast cancer (9F3) | 1.6 | 0.7 |
| 153432_Breast cancer (CHTN24652) | 15.6 | 6.2 |
| 153635_Breast cancer (D3C) | 0.6 | 0.4 |
| 164667_Breast cancer (5785) | 1.6 | 12.2 |
| 164676_Breast cancer (5070) | 1.5 | 0.6 |
| 164684_Breast cancer (6509) | 8.8 | 3.5 |
| 97764_Breast cancer node | 4.4 | 4.8 |
| metastasis (OD06083) | | |
| 108847_Breast cancer | 2.8 | 1.2 |
| 116421_Breast cancer (6314) | 0.1 | 0.0 |
| 145854_Breast cancer (9B8) | 1.2 | 0.4 |
| 153627_Breast cancer (D34) | 5.8 | 3.6 |
| 153636_Breast cancer (D3D) | 7.8 | 4.9 |
| 164668_Breast cancer (6314) | 0.5 | 0.2 |
| 164677_Breast cancer (5272) | 21.6 | 17.3 |
| 164685_Breast cancer (0170) | 80.1 | 50.7 |
| 98857_Breast cancer (OD06397-12) | 2.3 | 1.2 |
| 111288_Breast NAT (3367) | 14.6 | 6.2 |
| 116424_Breast cancer 4B* | 0.2 | 0.2 |
| 153628_Breast cancer (D35) | 18.9 | 11.7 |
| 153637_Breast cancer (D3E) | 1.3 | 0.9 |
| 164669_Breast cancer (6992) | 16.0 | 9.6 |
| 164678_Breast cancer (5297) | 50.0 | 42.6 |
| 164686_Breast cancer (0732) | 0.8 | 0.5 |
| 105687_Breast cancer 1B | 0.1 | 0.1 |
| 111289_Breast cancer 3B* | 8.2 | 5.7 |
| 116425_Breast NAT 4A | 0.3 | 0.3 |
| 145857_Breast cancer (9F0) | 8.8 | 8.0 |
| 153630_Breast cancer (D37) | 2.0 | 2.0 |
| 153638_Breast cancer (D3F) | 11.0 | 10.0 |
| 164670_Breast cancer (7078) | 1.7 | 2.7 |
| 164679_Breast cancer (5486) | 15.8 | 13.9 |
| 164687_Breast cancer (5881) | 11.2 | 4.0 |
| 105688_Breast NAT 1A | 0.2 | 0.1 |
| 111290_Breast NAT 3A* | 4.2 | 6.8 |
| 145846_Breast cancer (9B7) | 7.1 | 9.6 |
| 145858_Breast cancer (9B4) | 0.9 | 1.4 |
| 153631_Breast cancer (D38) | 0.3 | 0.2 |
| 153639_Breast cancer (D40) | 66.0 | 29.3 |
| 164671_Breast cancer (7082) | 62.4 | 50.0 |
| 164680_Breast cancer (5705) | 1.7 | 1.3 |
| 164688_Breast cancer (7222) | 1.5 | 0.5 |

Column A - Rel. Exp. (%) Ag1395, Run 399018961
Column B - Rel. Exp. (%) Ag8392, Run 398657263

TABLE XD
Ardais Panel 1.1

| Tissue Name | A | B |
|---|---|---|
| Lung adenocarcinoma SI A | 15.6 | 8.7 |
| Lung adenocarcinoma SI B | 22.2 | 75.3 |
| Lung adenocarcinoma SI B NAT | 100.0 | 70.7 |
| Lung adenocarcinoma SI C | 1.6 | 3.4 |
| Lung adenocarcinoma SI C NAT | 98.6 | 100.0 |
| Lung adenocarcinoma SII A | 11.7 | 14.5 |
| Lung adenocarcinoma SII A NAT | 58.6 | 76.3 |
| Lung adenocarcinoma SII C NAT | 62.4 | 55.5 |
| Lung adenocarcinoma SIII A | 41.5 | 42.9 |
| Lung adenocarcinoma SIII B | 32.1 | 33.7 |
| Lung adenocarcinoma SIII C | 16.8 | 25.2 |
| Lung SCC SI A | 27.9 | 74.7 |
| Lung SCC SI B NAT | 77.4 | 33.4 |
| Lung SCC SI C | 23.3 | 31.0 |
| Lung SCC SI C NAT | 71.2 | 82.9 |
| Lung SCC SI D | 40.6 | 62.4 |
| Lung SCC SI D NAT | 32.5 | 59.9 |
| Lung SCC SII A | 15.4 | 60.3 |
| Lung SCC SII B | 7.0 | 11.7 |
| Lung SCC SIII A | 11.5 | 11.5 |
| Lung SCC SIII A NAT | 29.1 | 13.4 |

Column A - Rel. Exp. (%) Ag1395, Run 332737494
Column B - Rel. Exp. (%) Ag8392, Run 369781003

TABLE XE
Ardais Prostate 1.0

| Tissue Name | A | B |
|---|---|---|
| 145904_Prostate cancer(9E2) | 100.0 | 100.0 |
| 149776_Prostate cancer(ADS) | 17.4 | 20.0 |
| 151135_Prostate NAT(B87) | 0.4 | 0.2 |
| 151143_Prostate NAT(B8A) | 0.3 | 0.2 |
| 153653_Prostate cancer(D4E) | 0.7 | 0.7 |
| 153661_Prostate cancer(D56) | 1.2 | 0.7 |
| 153669_Prostate NAT(D5E) | 1.2 | 0.6 |
| 153677_Prostate NAT(D66) | 0.2 | 0.1 |
| 153685_Prostate NAT(D6E) | 0.2 | 0.1 |
| 145905_Prostate NAT(A0C) | 1.0 | 0.7 |
| 151128_Prostate cancer(B8C) | 0.6 | 0.9 |
| 151136_Prostate cancer(B8B) | 3.3 | 2.1 |
| 151144_Prostate cancer(B8F) | 0.4 | 0.2 |
| 153654_Prostate cancer(D4F) | 4.5 | 3.8 |
| 153662_Prostate cancer(D57) | 0.2 | 0.0 |
| 153670_Prostate NAT(D5F) | 1.1 | 0.6 |
| 153678_Prostate NAT(D67) | 0.3 | 0.1 |
| 153686_Prostate NAT(D6F) | 0.2 | 0.2 |
| 145906_Prostate NAT(A09) | 0.4 | 0.3 |
| 151129_Prostate NAT(B93) | 0.7 | 0.6 |
| 151137_Prostate NAT(B86) | 0.3 | 0.4 |
| 151145_Prostate NAT(B91) | 0.4 | 0.3 |
| 153655_Prostate cancer(D50) | 0.7 | 0.4 |
| 153663_Prostate cancer(D58) | 0.1 | 0.0 |
| 153671_Prostate NAT(D60) | 0.2 | 0.6 |
| 153679_Prostate NAT(D68) | 0.2 | 0.2 |
| 153687_Prostate NAT(D70) | 0.4 | 0.3 |
| 145907_Prostate cancer(A0A) | 0.4 | 0.3 |
| 151130_Prostate cancer(B90) | 1.4 | 1.1 |
| 151138_Prostate cancer(B8D) | 11.6 | 11.3 |
| 153648_Prostate cancer(D49) | 2.1 | 1.8 |
| 153656_Prostate cancer(D51) | 0.4 | 0.1 |
| 153664_Prostate cancer(D59) | 1.2 | 0.9 |
| 153672_Prostate NAT(D61) | 0.3 | 0.7 |
| 153680_Prostate NAT(D69) | 0.3 | 0.3 |
| 155799_Prostate cancer(EA8) | 2.6 | 3.8 |
| 145909_Prostate cancer(9E7) | 3.1 | 4.4 |
| 151131_Prostate NAT(B85) | 1.2 | 0.6 |
| 151139_Prostate NAT(B8E) | 0.4 | 0.3 |
| 153649_Prostate cancer(D4A) | 0.7 | 0.4 |
| 153657_Prostate cancer(D52) | 0.6 | 0.2 |
| 153665_Prostate cancer(D5A) | 34.6 | 25.7 |
| 153673_Prostate NAT(D62) | 0.6 | 0.3 |
| 153681_Prostate NAT(D6A) | 0.2 | 0.1 |
| 145910_Prostate NAT(9C3) | 1.0 | 0.4 |
| 151132_Prostate cancer(B88) | 12.9 | 7.4 |
| 151140_Prostate cancer(B95) | 0.8 | 0.4 |
| 153650_Prostate cancer(D4B) | 3.7 | 3.1 |
| 153658_Prostate cancer(D53) | 1.2 | 0.4 |
| 153666_Prostate cancer(D5B) | 15.7 | 14.3 |
| 153674_Prostate NAT(D63) | 1.1 | 0.3 |
| 153682_Prostate NAT(D6B) | 0.9 | 0.6 |
| 149773_Prostate NAT(AD8) | 0.0 | 0.0 |
| 151133_Prostate NAT(B94) | 0.5 | 0.4 |
| 151141_Prostate NAT(B96) | 1.1 | 0.8 |
| 153651_Prostate cancer(D4C) | 11.1 | 9.7 |
| 153659_Prostate cancer(D54) | 3.5 | 3.0 |
| 153667_Prostate cancer(D5C) | 0.6 | 0.3 |
| 153675_Prostate NAT(D64) | 1.2 | 0.4 |
| 153683_Prostate NAT(D6C) | 0.8 | 0.3 |
| 149774_Prostate cancer(AD7) | 0.9 | 1.1 |
| 151134_Prostate cancer(B92) | 0.4 | 0.2 |
| 151142_Prostate cancer(B89) | 40.9 | 40.9 |
| 153652_Prostate cancer(D4D) | 0.6 | 0.3 |
| 153660_Prostate cancer(D55) | 12.8 | 11.5 |
| 153668_Prostate NAT(D5D) | 0.4 | 0.3 |
| 153676_Prostate NAT(D65) | 0.7 | 0.4 |
| 153684_Prostate NAT(D6D) | 0.3 | 0.2 |

Column A - Rel. Exp. (%) Ag1395, Run 379587273
Column B - Rel. Exp. (%) Ag8392, Run 369780997

TABLE XF
General_screening_panel_v1.6

| Tissue Name | A |
|---|---|
| Adipose | 0.5 |
| Melanoma* Hs688(A).T | 0.0 |
| Melanoma* Hs688(B).T | 0.0 |
| Melanoma* M14 | 0.0 |
| Melanoma* LOXIMVI | 0.3 |
| Melanoma* SK-MEL-5 | 0.4 |
| Squamous cell carcinoma SCC-4 | 1.5 |
| Testis Pool | 1.3 |
| Prostate ca.* (bone met) PC-3 | 0.0 |
| Prostate Pool | 0.2 |
| Placenta | 0.1 |
| Uterus Pool | 0.2 |
| Ovarian ca. OVCAR-3 | 0.1 |
| Ovarian ca. SK-OV-3 | 1.3 |
| Ovarian ca. OVCAR-4 | 1.0 |
| Ovarian ca. OVCAR-5 | 39.8 |
| Ovarian ca. IGROV-1 | 21.0 |
| Ovarian ca. OVCAR-8 | 3.4 |
| Ovary | 0.5 |
| Breast ca. MCF-7 | 1.6 |
| Breast ca. MDA-MB-231 | 0.1 |
| Breast ca. BT 549 | 0.1 |
| Breast ca. T47D | 0.4 |
| Breast ca. MDA-N | 0.0 |
| Breast Pool | 0.6 |
| Trachea | 0.8 |
| Lung | 0.0 |
| Fetal Lung | 0.8 |
| Lung ca. NCI-N417 | 7.9 |
| Lung ca. LX-1 | 0.0 |
| Lung ca. NCI-H146 | 1.3 |
| Lung ca. SHP-77 | 6.2 |
| Lung ca. A549 | 0.2 |
| Lung ca. NCI-H526 | 3.0 |
| Lung ca. NCI-H23 | 1.6 |
| Lung ca. NCI-H460 | 0.5 |
| Lung ca. HOP-62 | 0.0 |
| Lung ca. NCI-H522 | 17.9 |
| Liver | 0.0 |
| Fetal Liver | 0.3 |
| Liver ca. HepG2 | 0.0 |
| Kidney Pool | 0.4 |
| Fetal Kidney | 0.2 |
| Renal ca. 786-0 | 1.4 |
| Renal ca. A498 | 21.6 |
| Renal ca. ACHN | 6.2 |
| Renal ca. UO-31 | 0.6 |
| Renal ca. TK-10 | 10.9 |
| Bladder | 1.2 |
| Gastric ca. (liver met.) NCI-N87 | 0.0 |
| Gastric ca. KATO III | 0.0 |
| Colon ca. SW-948 | 0.0 |
| Colon ca. SW480 | 6.6 |
| Colon ca.* (SW480 met) SW620 | 0.8 |
| Colon ca. HT29 | 0.3 |
| Colon ca. HCT-116 | 15.5 |
| Colon ca. CaCo-2 | 0.6 |
| Colon cancer tissue | 0.6 |
| Colon ca. SW1116 | 4.0 |
| Colon ca. Colo-205 | 0.1 |
| Colon ca. SW-48 | 0.0 |
| Colon Pool | 0.8 |
| Small Intestine Pool | 0.4 |
| Stomach Pool | 0.5 |
| Bone Marrow Pool | 0.2 |
| Fetal Heart | 0.2 |
| Heart Pool | 0.1 |
| Lymph Node Pool | 0.8 |
| Fetal Skeletal Muscle | 0.8 |
| Skeletal Muscle Pool | 0.1 |
| Spleen Pool | 1.0 |
| Thymus Pool | 1.5 |
| CNS cancer (glio/astro) U87-MG | 0.0 |
| CNS cancer (glio/astro) U-118-MG | 0.1 |
| CNS cancer (neuro; met) SK-N-AS | 2.5 |
| CNS cancer (astro) SF-539 | 0.0 |
| CNS cancer (astro) SNB-75 | 0.9 |
| CNS cancer (glio) SNB-19 | 19.5 |
| CNS cancer (glio) SF-295 | 0.7 |
| Brain (Amygdala) Pool | 38.4 |
| Brain (cerebellum) | 100.0 |
| Brain (fetal) | 62.4 |
| Brain (Hippocampus) Pool | 36.9 |
| Cerebral Cortex Pool | 72.2 |
| Brain (Substantia nigra) Pool | 48.0 |
| Brain (Thalamus) Pool | 46.7 |
| Brain (whole) | 72.2 |
| Spinal Cord Pool | 3.2 |
| Adrenal Gland | 11.3 |
| Pituitary gland Pool | 2.8 |
| Salivary Gland | 0.5 |
| Thyroid (female) | 0.1 |
| Pancreatic ca. CAPAN2 | 1.0 |
| Pancreas Pool | 0.3 |

Column A - Rel. Exp. (%) Ag1395, Run 277226632

TABLE XG
HASS Panel v1.0

| Tissue Name | A |
|---|---|
| MCF-7 C1 | 7.5 |
| MCF-7 C2 | 7.1 |
| MCF-7 C3 | 4.0 |
| MCF-7 C4 | 12.5 |
| MCF-7 C5 | 9.1 |
| MCF-7 C6 | 12.5 |
| MCF-7 C7 | 5.2 |
| MCF-7 C9 | 5.0 |
| MCF-7 C10 | 8.2 |
| MCF-7 C11 | 1.2 |
| MCF-7 C12 | 5.1 |
| MCF-7 C13 | 2.1 |
| MCF-7 C15 | 3.6 |
| MCF-7 C16 | 13.9 |
| MCF-7 C17 | 8.9 |
| T24 D1 | 0.1 |
| T24 D2 | 0.1 |
| T24 D3 | 0.1 |
| T24 D4 | 0.4 |
| T24 D5 | 0.2 |
| T24 D6 | 0.1 |
| T24 D7 | 0.2 |
| T24 D9 | 0.1 |
| T24 D10 | 0.0 |
| T24 D11 | 0.1 |
| T24 D12 | 0.0 |
| T24 D13 | 0.2 |
| T24 D15 | 0.1 |
| T24 D16 | 0.0 |
| T24 D17 | 0.1 |
| CAPaN B1 | 13.0 |
| CAPaN B2 | 11.6 |
| CAPaN B3 | 5.0 |
| CAPaN B4 | 12.7 |
| CAPaN B5 | 18.4 |
| CAPaN B6 | 11.2 |
| CAPaN B7 | 3.8 |
| CAPaN B8 | 3.7 |
| CAPaN B9 | 7.5 |
| CAPaN B10 | 20.0 |
| CAPaN B11 | 26.8 |
| CAPaN B12 | 14.6 |
| CAPaN B13 | 5.1 |
| CAPaN B14 | 5.9 |
| CAPaN B15 | 8.0 |
| CAPaN B16 | 13.8 |
| CAPaN B17 | 13.7 |
| U87-MG F1 (B) | 0.0 |
| U87-MG F2 | 0.0 |
| U87-MG F3 | 0.1 |
| U87-MG F4 | 0.0 |
| U87-MG F5 | 0.0 |
| U87-MG F6 | 0.1 |
| U87-MG F7 | 0.0 |
| U87-MG F8 | 0.1 |
| U87-MG F9 | 0.1 |
| U87-MG F10 | 0.0 |
| U87-MG F11 | 0.0 |
| U87-MG F12 | 0.0 |
| U87-MG F13 | 0.0 |
| U87-MG F14 | 0.0 |
| U87-MG F15 | 0.0 |
| U87-MG F16 | 0.0 |
| U87-MG F17 | 0.1 |
| LnCAP A1 | 0.9 |
| LnCAP A2 | 0.5 |
| LnCAP A3 | 0.9 |
| LnCAP A4 | 1.0 |
| LnCAP A5 | 1.2 |
| LnCAP A6 | 0.4 |
| LnCAP A7 | 0.7 |
| LnCAP A8 | 1.2 |
| LnCAP A9 | 1.1 |
| LnCAP A10 | 1.0 |
| LnCAP A11 | 1.6 |
| LnCAP A12 | 0.0 |
| LnCAP A13 | 0.4 |
| LnCAP A14 | 1.2 |
| LnCAP A15 | 0.3 |
| LnCAP A16 | 1.1 |
| LnCAP A17 | 1.3 |
| Primary Astrocytes | 0.2 |
| Primary Renal Proximal Tubule Epithelial cell A2 | 1.5 |
| Primary melanocytes A5 | 0.0 |
| 126443 - 341 medullo | 0.0 |
| 126444 - 487 medullo | 0.5 |
| 126445 - 425 medullo | 0.0 |
| 126446 - 690 medullo | 100.0 |
| 126447 - 54 adult glioma | 0.8 |
| 126448 - 245 adult glioma | 0.1 |
| 126449 - 317 adult glioma | 0.1 |
| 126450 - 212 glioma | 0.0 |
| 126451 - 456 glioma | 0.1 |

Column A - Rel. Exp. (%) Ag1395, Run 268623664

TABLE XG
Panel 2D

| Tissue Name | A | B |
|---|---|---|
| Normal Colon | 4.2 | 1.9 |
| CC Well to Mod Diff (ODO3866) | 0.7 | 1.8 |
| CC Margin (ODO3866) | 0.0 | 1.0 |
| CC Gr.2 rectosigmoid (ODO3868) | 0.0 | 1.1 |
| CC Margin (ODO3868) | 0.0 | 0.0 |
| CC Mod Diff (ODO3920) | 0.0 | 1.2 |
| CC Margin (ODO3920) | 0.0 | 0.8 |
| CC Gr.2 ascend colon (ODO3921) | 0.9 | 2.3 |
| CC Margin (ODO3921) | 0.0 | 0.4 |
| CC from Partial Hepatectomy | 0.7 | 0.2 |
| (ODO4309) Mets | | |
| Liver Margin (ODO4309) | 0.0 | 0.9 |
| Colon mets to lung (OD04451-01) | 0.0 | 2.3 |
| Lung Margin (OD04451-02) | 0.8 | 0.0 |
| Normal Prostate 6546-1 | 9.0 | 8.2 |
| Prostate Cancer (OD04410) | 0.0 | 3.5 |
| Prostate Margin (OD04410) | 2.0 | 1.7 |
| Prostate Cancer (OD04720-01) | 0.8 | 1.7 |
| Prostate Margin (OD04720-02) | 0.0 | 1.5 |
| Normal Lung 061010 | 3.1 | 10.9 |
| Lung Met to Muscle (ODO4286) | 4.4 | 4.1 |
| Muscle Margin (ODO4286) | 0.0 | 0.5 |
| Lung Malignant Cancer (OD03126) | 2.2 | 2.5 |
| Lung Margin (OD03126) | 3.2 | 4.6 |
| Lung Cancer (OD04404) | 2.4 | 1.1 |
| Lung Margin (OD04404) | 3.3 | 4.2 |
| Lung Cancer (OD04565) | 0.0 | 1.6 |
| Lung Margin (OD04565) | 1.7 | 1.7 |
| Lung Cancer (OD04237-01) | 0.8 | 4.5 |
| Lung Margin (OD04237-02) | 3.8 | 7.1 |
| Ocular Mel Met to Liver (ODO4310) | 0.0 | 0.0 |
| Liver Margin (ODO4310) | 6.2 | 2.1 |
| Melanoma Mets to Lung (OD04321) | 0.8 | 0.0 |
| Lung Margin (OD04321) | 3.8 | 5.3 |
| Normal Kidney | 0.8 | 1.6 |
| Kidney Ca, Nuclear grade 2 (OD04338) | 1.2 | 2.8 |
| Kidney Margin (OD04338) | 0.0 | 1.8 |
| Kidney Ca Nuclear grade 1/2 | 5.9 | 5.4 |
| (OD04339) | | |
| Kidney Margin (OD04339) | 0.0 | 0.0 |
| Kidney Ca, Clear cell type (OD04340) | 1.3 | 7.4 |
| Kidney Margin (OD04340) | 0.0 | 0.3 |
| Kidney Ca, Nuclear grade 3 (OD04348) | 0.0 | 2.1 |
| Kidney Margin (OD04348) | 0.8 | 0.8 |
| Kidney Cancer (OD04622-01) | 2.2 | 4.0 |
| Kidney Margin (OD04622-03) | 0.7 | 0.4 |
| Kidney Cancer (OD04450-01) | 0.0 | 0.4 |
| Kidney Margin (OD04450-03) | 0.0 | 0.0 |
| Kidney Cancer 8120607 | 27.9 | 60.3 |
| Kidney Margin 8120608 | 0.8 | 2.1 |
| Kidney Cancer 8120613 | 0.8 | 1.7 |
| Kidney Margin 8120614 | 0.7 | 0.7 |
| Kidney Cancer 9010320 | 4.7 | 6.3 |
| Kidney Margin 9010321 | 0.0 | 2.7 |
| Normal Uterus | 0.0 | 2.2 |
| Uterus Cancer 064011 | 0.0 | 8.9 |
| Normal Thyroid | 8.7 | 1.2 |
| Thyroid Cancer 064010 | 0.0 | 0.0 |
| Thyroid Cancer A302152 | 0.0 | 2.5 |
| Thyroid Margin A302153 | 1.1 | 0.8 |
| Normal Breast | 2.8 | 4.1 |
| Breast Cancer (OD04566) | 0.0 | 1.8 |
| Breast Cancer (OD04590-01) | 28.3 | 27.4 |
| Breast Cancer Mets (OD04590-03) | 13.3 | 14.2 |
| Breast Cancer Metastasis (OD04655-05) | 37.9 | 100.0 |
| Breast Cancer 064006 | 12.0 | 19.2 |
| Breast Cancer 1024 | 33.9 | 25.2 |
| Breast Cancer 9100266 | 6.7 | 7.6 |
| Breast Margin 9100265 | 0.5 | 9.2 |
| Breast Cancer A209073 | 3.7 | 6.8 |
| Breast Margin A209073 | 0.7 | 0.0 |
| Normal Liver | 0.0 | 2.6 |
| Liver Cancer 064003 | 0.0 | 1.3 |
| Liver Cancer 1025 | 0.4 | 2.0 |
| Liver Cancer 1026 | 0.0 | 1.6 |
| Liver Cancer 6004-T | 1.6 | 3.3 |
| Liver Tissue 6004-N | 1.4 | 0.7 |
| Liver Cancer 6005-T | 0.8 | 0.8 |
| Liver Tissue 6005-N | 0.0 | 0.0 |
| Normal Bladder | 3.5 | 3.8 |
| Bladder Cancer 1023 | 0.8 | 0.5 |
| Bladder Cancer A302173 | 3.2 | 1.1 |
| Bladder Cancer (OD04718-01) | 3.8 | 2.3 |
| Bladder Normal Adjacent (OD04718-03) | 5.2 | 7.4 |
| Normal Ovary | 3.0 | 2.9 |
| Ovarian Cancer 064008 | 3.2 | 2.9 |
| Ovarian Cancer (OD04768-07) | 3.5 | 4.6 |
| Ovary Margin (OD04768-08) | 0.9 | 2.2 |
| Normal Stomach | 2.7 | 3.7 |
| Gastric Cancer 9060358 | 0.4 | 0.2 |
| Stomach Margin 9060359 | 4.3 | 1.3 |
| Gastric Cancer 9060395 | 3.0 | 1.2 |
| Stomach Margin 9060394 | 2.5 | 1.0 |
| Gastric Cancer 9060397 | 100.0 | 48.0 |
| Stomach Margin 9060396 | 1.0 | 2.2 |
| Gastric Cancer 064005 | 4.9 | 6.7 |

Column A - Rel. Exp. (%) Ag1395, Run 145711201
Column B - Rel. Exp. (%) Ag1395, Run 162599933

TABLE XI
Panel 3D

| Tissue Name | A |
|---|---|
| Daoy- Medulloblastoma | 0.0 |
| TE671- Medulloblastoma | 0.6 |
| D283 Med- Medulloblastoma | 0.5 |
| PFSK-1- Primitive Neuroectodermal | 0.0 |
| XF-498- CNS | 0.6 |
| SNB-78- Glioma | 0.0 |
| SF-268- Glioblastoma | 0.0 |
| T98G- Glioblastoma | 6.5 |
| SK-N-SH- Neuroblastoma (metastasis) | 10.3 |
| SF-295- Glioblastoma | 0.8 |
| Cerebellum | 46.0 |
| Cerebellum | 41.5 |
| NCI-H292- Mucoepidermoid lung carcinoma | 0.4 |
| DMS-114- Small cell lung cancer | 9.0 |
| DMS-79- Small cell lung cancer | 100.0 |
| NCI-H146- Small cell lung cancer | 4.2 |
| NCI-H526- Small cell lung cancer | 16.5 |
| NCI-N417- Small cell lung cancer | 22.8 |
| NCI-H82- Small cell lung cancer | 9.5 |
| NCI-H157- Squamous cell lung cancer (metastasis) | 0.0 |
| NCI-H1155- Large cell lung cancer | 27.7 |
| NCI-H1299- Large cell lung cancer | 0.0 |
| NCI-H727- Lung carcinoid | 14.5 |
| NCI-UMC-11- Lung carcinoid | 22.1 |
| LX-1- Small cell lung cancer | 0.2 |
| Colo-205- Colon cancer | 0.2 |
| KM12- Colon cancer | 0.0 |
| KM20L2- Colon cancer | 0.2 |
| NCI-H716- Colon cancer | 0.4 |
| SW-48- Colon adenocarcinoma | 0.0 |
| SW1116- Colon adenocarcinoma | 7.2 |
| LS 174T- Colon adenocarcinoma | 0.9 |
| SW-948- Colon adenocarcinoma | 0.0 |
| SW-480- Colon adenocarcinoma | 0.0 |
| NCI-SNU-5- Gastric carcinoma | 3.5 |
| KATO III- Gastric carcinoma | 0.3 |
| NCI-SNU-16- Gastric carcinoma | 2.1 |
| NCI-SNU-1- Gastric carcinoma | 0.0 |
| RF-1- Gastric adenocarcinoma | 0.0 |
| RF-48- Gastric adenocarcinoma | 0.0 |
| MKN-45- Gastric carcinoma | 2.9 |
| NCI-N87- Gastric carcinoma | 0.0 |
| OVCAR-5- Ovarian carcinoma | 11.2 |
| RL95-2- Uterine carcinoma | 0.0 |
| HelaS3- Cervical adenocarcinoma | 0.0 |
| Ca Ski- Cervical epidermoid carcinoma | 0.0 |
| (metastasis) | |
| ES-2- Ovarian clear cell carcinoma | 0.2 |
| Ramos- Stimulated with PMA/ionomycin 6 h | 0.0 |
| Ramos- Stimulated with PMA/ionomycin 14 h | 0.0 |
| MEG-01- Chronic myelogenous leukemia | 0.0 |
| (megokaryoblast) | |
| Raji- Burkitt's lymphoma | 0.0 |
| Daudi- Burkitt's lymphoma | 0.0 |
| U266- B-cell plasmacytoma | 0.0 |
| CA46- Burkitt's lymphoma | 0.0 |
| RL- non-Hodgkin's B-cell lymphoma | 0.0 |
| JM1- pre-B-cell lymphoma | 0.0 |
| Jurkat- T cell leukemia | 0.0 |
| TF-1- Erythroleukemia | 0.0 |
| HUT 78- T-cell lymphoma | 0.0 |
| U937- Histiocytic lymphoma | 5.3 |
| KU-812- Myelogenous leukemia | 0.0 |
| 769-P- Clear cell renal carcinoma | 6.3 |
| Caki-2- Clear cell renal carcinoma | 15.8 |
| SW 839- Clear cell renal carcinoma | 5.9 |
| Rhabdoid kidney tumor | 1.2 |
| Hs766T- Pancreatic carcinoma (LN | 0.0 |
| metastasis) | |
| CAPAN-1- Pancreatic adenocarcinoma (liver | 27.7 |
| metastasis) | |
| SU86.86- Pancreatic carcinoma (liver | 11.0 |
| metastasis) | |
| BxPC-3- Pancreatic adenocarcinoma | 0.0 |
| HPAC- Pancreatic adenocarcinoma | 0.0 |
| MIA PaCa-2- Pancreatic carcinoma | 0.0 |
| CFPAC-1- Pancreatic ductal adenocarcinoma | 2.8 |
| PANC-1- Pancreatic epithelioid ductal | 0.2 |
| carcinoma | |
| T24- Bladder carcinma (transitional cell) | 0.0 |
| 5637- Bladder carcinoma | 0.2 |
| HT-1197- Bladder carcinoma | 0.0 |
| UM-UC-3- Bladder carcinma (transitional cell) | 0.3 |
| A204- Rhabdomyosarcoma | 1.0 |
| HT-1080- Fibrosarcoma | 0.0 |
| MG-63- Osteosarcoma | 0.2 |
| SK-LMS-1- Leiomyosarcoma (vulva) | 0.0 |
| SJRH30- Rhabdomyosarcoma (met to bone | 0.1 |
| marrow) | |
| A431- Epidermoid carcinoma | 0.0 |
| WM266-4- Melanoma | 0.0 |
| DU 145- Prostate carcinoma (brain | 0.0 |
| metastasis) | |
| MDA-MB-468- Breast adenocarcinoma | 9.2 |
| SCC-4- Squamous cell carcinoma of tongue | 0.0 |
| SCC-9- Squamous cell carcinoma of tongue | 0.0 |
| SCC-15- Squamous cell carcinoma of tongue | 0.0 |
| CAL 27- Squamous cell carcinoma of tongue | 4.0 |

Column A - Rel. Exp. (%) Ag1395, Run 163477780

TABLE XJ
Panel 4D

| Tissue Name | A | B |
|---|---|---|
| Secondary Th1 act | 0.0 | 1.5 |
| Secondary Th2 act | 0.0 | 0.0 |
| Secondary Tr1 act | 0.0 | 0.0 |
| Secondary Th1 rest | 0.0 | 0.0 |
| Secondary Th2 rest | 0.0 | 0.0 |
| Secondary Tr1 rest | 0.0 | 0.0 |
| Primary Th1 act | 0.0 | 0.0 |
| Primary Th2 act | 0.0 | 0.0 |
| Primary Tr1 act | 1.6 | 0.0 |
| Primary Th1 rest | 1.7 | 0.0 |
| Primary Th2 rest | 0.0 | 0.0 |
| Primary Tr1 rest | 1.3 | 0.0 |
| CD45RA CD4 lymphocyte act | 0.0 | 0.7 |
| CD45RO CD4 lymphocyte act | 1.2 | 0.0 |
| CD8 lymphocyte act | 0.0 | 0.0 |
| Secondary CD8 lymphocyte rest | 1.2 | 0.0 |
| Secondary CD8 lymphocyte act | 0.0 | 0.0 |
| CD4 lymphocyte none | 0.0 | 0.0 |
| 2ry Th1/Th2/Tr1_anti-CD95 CH11 | 1.6 | 0.0 |
| LAK cells rest | 41.8 | 49.7 |
| LAK cells IL-2 | 0.0 | 0.0 |
| LAK cells IL-2 + IL-12 | 0.0 | 0.0 |
| LAK cells IL-2 + IFN gamma | 4.2 | 2.9 |
| LAK cells IL-2 + IL-18 | 2.7 | 1.5 |
| LAK cells PMA/ionomycin | 63.3 | 100.0 |
| NK Cells IL-2 rest | 0.0 | 0.0 |
| Two Way MLR 3 day | 21.8 | 16.7 |
| Two Way MLR 5 day | 4.6 | 2.2 |
| Two Way MLR 7 day | 5.0 | 2.5 |
| PBMC rest | 3.6 | 9.5 |
| PBMC PWM | 0.0 | 97.9 |
| PBMC PHA-L | 2.9 | 2.7 |
| Ramos (B cell) none | 0.0 | 0.0 |
| Ramos (B cell) ionomycin | 0.0 | 0.0 |
| B lymphocytes PWM | 13.8 | 0.0 |
| B lymphocytes CD40L and IL-4 | 0.0 | 0.0 |
| EOL-1 dbcAMP | 0.0 | 4.4 |
| EOL-1 dbcAMP PMA/ionomycin | 0.0 | 1.3 |
| Dendritic cells none | 31.9 | 34.4 |
| Dendritic cells LPS | 36.3 | 23.3 |
| Dendritic cells anti-CD40 | 39.8 | 40.3 |
| Monocytes rest | 55.9 | 58.6 |
| Monocytes LPS | 13.3 | 11.3 |
| Macrophages rest | 93.3 | 96.6 |
| Macrophages LPS | 38.7 | 28.3 |
| HUVEC none | 0.0 | 4.0 |
| HUVEC starved | 2.6 | 0.0 |
| HUVEC IL-1beta | 0.0 | 1.4 |
| HUVEC IFN gamma | 1.5 | 6.3 |
| HUVEC TNF alpha + IFN gamma | 0.0 | 0.0 |
| HUVEC TNF alpha + IL4 | 0.0 | 0.0 |
| HUVEC IL-11 | 1.4 | 0.0 |
| Lung Microvascular EC none | 0.0 | 1.4 |
| Lung Microvascular EC TNFalpha + IL-1beta | 0.0 | 0.7 |
| Microvascular Dermal EC none | 11.0 | 5.5 |
| Microsvasular Dermal EC TNFalpha + IL-1beta | 7.6 | 17.4 |
| Bronchial epithelium TNFalpha + IL1beta | 0.0 | 0.0 |
| Small airway epithelium none | 0.0 | 0.0 |
| Small airway epithelium TNFalpha + IL-1beta | 1.1 | 1.9 |
| Coronery artery SMC rest | 0.0 | 0.0 |
| Coronery artery SMC TNFalpha + IL-1beta | 0.0 | 0.0 |
| Astrocytes rest | 1.5 | 0.0 |
| Astrocytes TNFalpha + IL-1beta | 0.0 | 1.2 |
| KU-812 (Basophil) rest | 0.0 | 0.0 |
| KU-812 (Basophil) PMA/ionomycin | 0.0 | 2.6 |
| CCD1106 (Keratinocytes) none | 0.0 | 1.1 |
| CCD1106 (Keratinocytes) TNFalpha + IL-1beta | 12.5 | 12.3 |
| Liver cirrhosis | 23.8 | 21.0 |
| Lupus kidney | 3.0 | 2.0 |
| NCI-H292 none | 1.1 | 5.8 |
| NCI-H292 IL-4 | 2.1 | 6.4 |
| NCI-H292 IL-9 | 3.8 | 0.0 |
| NCI-H292 IL-13 | 0.0 | 4.2 |
| NCI-H292 IFN gamma | 7.1 | 7.1 |
| HPAEC none | 10.4 | 13.4 |
| HPAEC TNF alpha + IL-1 beta | 0.0 | 0.0 |
| Lung fibroblast none | 0.0 | 0.0 |
| Lung fibroblast TNF alpha + IL-1 beta | 0.0 | 0.0 |
| Lung fibroblast IL-4 | 0.0 | 0.0 |
| Lung fibroblast IL-9 | 0.0 | 5.5 |
| Lung fibroblast IL-13 | 1.3 | 0.0 |
| Lung fibroblast IFN gamma | 0.0 | 0.0 |
| Dermal fibroblast CCD1070 rest | 0.0 | 0.0 |
| Dermal fibroblast CCD1070 TNF alpha | 0.0 | 0.0 |
| Dermal fibroblast CCD1070 IL-1 beta | 0.0 | 5.6 |
| Dermal fibroblast IFN gamma | 0.0 | 0.0 |
| Dermal fibroblast IL-4 | 0.0 | 0.0 |
| IBD Colitis 2 | 0.0 | 1.5 |
| IBD Crohn's | 1.4 | 0.0 |
| Colon | 21.3 | 19.2 |
| Lung | 100.0 | 95.3 |
| Thymus | 6.9 | 1.2 |
| Kidney | 8.8 | 3.8 |

Column A - Rel. Exp. (%) Ag1395, Run 140112952
Column B - Rel. Exp. (%) Ag1395, Run 142259379

TABLE XK
Panel 5 Islet

| Tissue Name | A |
|---|---|
| 97457_Patient-02go_adipose | 15.1 |
| 97476_Patient-07sk_skeletal muscle | 0.0 |
| 97477_Patient-07ut_uterus | 3.9 |
| 97478_Patient-07pl_placenta | 0.0 |
| 99167_Bayer Patient 1 | 62.0 |
| 97482_Patient-08ut_uterus | 8.2 |
| 97483_Patient-08pl_placenta | 0.0 |
| 97486_Patient-09sk_skeletal muscle | 0.0 |
| 97487_Patient-09ut_uterus | 3.7 |
| 97488_Patient-09pl_placenta | 0.0 |
| 97492_Patient-10ut_uterus | 9.7 |
| 97493_Patient-10pl_placenta | 0.0 |
| 97495_Patient-11go_adipose | 17.2 |
| 97496_Patient-11sk_skeletal muscle | 0.0 |
| 97497_Patient-11ut_uterus | 14.7 |
| 97498_Patient-11pl_placenta | 0.0 |
| 97500_Patient-12go_adipose | 19.6 |
| 97501_Patient-12sk_skeletal muscle | 0.0 |
| 97502_Patient-12ut_uterus | 27.0 |
| 97503_Patient-12pl_placenta | 1.6 |
| 94721_Donor 2 U - A_Mesenchymal Stem Cells | 0.0 |
| 94722_Donor 2 U - B_Mesenchymal Stem Cells | 0.0 |
| 94723_Donor 2 U - C_Mesenchymal Stem Cells | 0.0 |
| 94709_Donor 2 AM - A_adipose | 0.0 |
| 94710_Donor 2 AM - B_adipose | 0.0 |
| 94711_Donor 2 AM - C_adipose | 0.0 |
| 94712_Donor 2 AD - A_adipose | 0.0 |
| 94713_Donor 2 AD - B_adipose | 0.0 |
| 94714_Donor 2 AD - C_adipose | 0.0 |
| 94742_Donor 3 U - A_Mesenchymal Stem Cells | 0.0 |
| 94743_Donor 3 U - B_Mesenchymal Stem Cells | 0.0 |
| 94730_Donor 3 AM - A_adipose | 0.0 |
| 94731_Donor 3 AM - B_adipose | 0.0 |
| 94732_Donor 3 AM - C_adipose | 0.0 |
| 94733_Donor 3 AD - A_adipose | 0.0 |
| 94734_Donor 3 AD - B_adipose | 0.0 |
| 94735_Donor 3 AD - C_adipose | 0.0 |
| 77138_Liver_HepG2 untreated | 0.0 |
| 73556_Heart_Cardiac stromal cells (primary) | 3.2 |
| 81735_Small Intestine | 5.4 |
| 72409_Kidney_Proximal Convoluted Tubule | 28.1 |
| 82685_Small intestine_Duodenum | 0.0 |
| 90650_Adrenal_Adrenocortical adenoma | 0.0 |
| 72410_Kidney_HRCE | 42.6 |
| 72411_Kidney_HRE | 100.0 |
| 73139_Uterus_Uterine smooth muscle cells | 0.0 |

Column A - Rel. Exp. (%) Ag1395, Run 267095054

Ardais Breast1.0 Summary: Ag1395/Ag8392 Highest expression of this gene was detected in abreast cancer sample (CTs=25-27). Moderate to high levels of expression of this gene were seen in normal and cancer samples from breast. Expression of this gene was higher in a number of cancer samples. Therefore, therapeutic modulation of this gene, expressed protein and/or use of antibodies or small molecule targeting this gene or gene product is useful in the treatment of breast cancer.

Ardais Panel 1.1 Summary: Ag1395/Ag8392 Highest expression of this gene was detected in normal adjacent lung tissue (CTs=29-30). Moderate to high levels of expression of this gene were seen in normal and cancer samples from lung. Expression of this gene was higher in a number of normal adjacent tissues. Therefore, therapeutic modulation of this gene, expressed protein and/or use of antibodies or small molecule targeting this gene or gene product is useful in the treatment of lung cancer.

Ardais Prostate 1.0 Summary: Ag1395/Ag8392 Highest expression of this gene was detected in a prostate cancer sample (CTs=25). Moderate to high levels of expression of this gene were seen in normal and cancer samples from prostate. Expression of this gene was higher in a number of prostate cancer samples. Therefore, therapeutic modulation of this gene, expressed protein and/or use of antibodies or small molecule targeting this gene or gene product is useful in the treatment of prostate cancer.

General_screening₁₃ panel_v1.6 Summary: Ag1395 Highest expression of this gene was detected in cerebellum (CT=26). This gene showed a brain preferential expression, with high expression of this gene seen in all the regions of the brain examined including amygdala, cerebellum, hippocampus, cerebral cortex pool, substantia nigra, and thalamus. This gene encodes an UNC5H1 receptor, an ortholog of Caenorhabditis elegans UNC5, which is essential for dorsal guidance of pioneer axons and for the movement of cells away from the netrin ligand (Ackerman, S. L. Nature 386: 838-842). The family of Netrin ligands, netrin-1-2-3 and β-netrin, key regulators of neuronal cell migration and axonal guidance bind to the DCC (deleted in colorectal cancer) family and the UNC5 family (UNC5H1, UNC5H2 and UNC5H3). The DCC family of proteins is implicated in axon attraction and those of the UNC5 family in axon repulsion. Mice homozygous for the spontaneous rostral cerebellar malformation mutation (rcm(s)) homolog of UNC-5 exhibit cerebellar and midbrain defects as a result of abnormalities in the postnatal cerebellar neuronal migration. Modulation of the expression of this gene, expressed protein and/or use of antibodies/small molecule targeting this gene or gene product is useful in the treatment of cerbellar malformation and other brain related disorders including Alzheimer's disease, Parkinson's disease, epilepsy, multiple sclerosis, schizophrenia and depression.

Significant expression of this gene was also seen in a number of cancer cell lines derived from pancreatic, lung, brain, colon, renal, and ovarian cancers. UNC5 receptors have been shown to create cellular states of dependence on their respective ligands by inhibiting apoptosis in the presence of ligand, but inducing apoptosis when unoccupied by ligand. UNC5H proteins are caspase substrates in vitro and caspase cleavage is required for cell death induction (Llambi F. EMBO J 20(11):2715-22). Based on the known role of these genes in cell migration and the expression of this gene cancer tissues and cell lines, including the metastatic cancer cell lines SW620 and SK-N-AS, the most likely mechanism for this gene is to increase the metastatic potential of tumor cells. When induced in endothelial cells, this gene will have a role in tumor induced endo cell migration and cell survival. The two roles might be interrelated as it is known that cells need to resist anoikis to survive during migration. Therefore, modulation of this gene, expressed protein and/or use of antibodies or small molecule targeting this gene or gene product is useful in the treatment of pancreatic, lung, brain, colon, renal, and ovarian cancers.

HASS Panel v1.0 Summary: Ag1395 Highest expression of this gene was detected in medullablastoma sample (CT=26.5). Low expression of this gene was also seen in a glioma sample. Therefore, therapeutic modulation of this gene or expressed protein is useful in the treatment of glioma and medulloblastoma.

In addition, significant expression of this gene was seen in samples from breast cancer MCF7 and pancreatic cancer CaPaN cell lines. The expression of this gene was not significantly changed by oxygen deprivation, acidic or a serum starved environment in the these two cell lines in this panel.

Panel 2D Summary: Ag1395 Highest expression of this gene was detected in a gastric cancer and metastatic breast cancer samples (CTs=29-31). Moderate to low levels of expression of this gene were also seen in number of kidney, breast and gastric cancer samples. Therefore, expression of this gene is useful as a marker to detect the presence of these cancers and therapeutic modulation of this gene, expressed protein and/or use of antibodies or small molecule is useful in the treatment of breast, kidney and gastric cancers.

Panel 3D Summary: Ag1395 Highest expression of this gene was detected in a lung cancer cell line (CT=28.8). Moderate to low levels of expression of this gene were detected in cancer cell lines derived from metastatic neuroblastoma, glioblastoma, lung, gastric, colon, lymphoma, renal, pancreatic, breast and tongue cancers. Therefore, therapeutic modulation of this gene and/or expressed protein is useful in the treatment of these cancers.

Panel 4D Summary: Ag1395 Highest expression of this gene was detected in lung and PMA/ionomycin activated LAK cells (CTs=31). Moderate to low levels of expression of this gene were also seen in resting LAK cells, resting and activated dendritic cells, monocytes and macrophages, activated keratinocytes, liver cirrhosis and normal colon tissues. Expression of this gene in monocytes, LAK, macrophage and dendritic cells demonstrates that the expressed protein is important in differentiation and activation of these cell types. Therefore, regulating the expression of this gene or expressed protein will alter the types and levels of monocytic cells regulated by cytokine and chemokine production and T cell activation. Therapeutics modulation of this gene, encoded protein and/or use of antibodies or small molecule targeting this gene or gene product is useful in the treatment of liver cirrhosis, asthma, emphysema, inflammatory bowel disease, arthritis and psoriasis.

Panel 5 Islet Summary: Ag1395 Expression of this gene was limited to the kidney on this panel (CT=33.9). Therefore, modulation of this gene and/or expressed protein is useful in the treatment of kidney related disorders including systemic lupus erythematosus and glomerulonephritis.

Y. CG53944-02: Novel Membrane Protein

Expression of full-length physical clone CG53944-02 was assessed using the primer-probe sets Ag2639, Ag2640, and Ag2641, described in Tables YA, YB, and YC. Results of the RTQ-PCR runs are shown in Tables YD, YE, YF and YG.

TABLE YA
Probe Name Ag2639

| | | | | SEQ |
|---|---|---|---|---|
| | | | Start | ID |
| Primers | Sequences | Length | Position | No |
| Forward | 5′-ctcatcctcagcaacaacca-3′ | 20 | 406 | 1372 |
| Probe | TET-5′-cctggatgattgtgccgagacact-3′- | 24 | 450 | 1373 |
| | TAMRA | | | |
| Reverse | 5′-taggagaggtcgaggtcctc-3′ | 20 | 475 | 1374 |

TABLE YB
Probe Name Ag2640

| | | | | SEQ |
|---|---|---|---|---|
| | | | Start | ID |
| Primers | Sequences | Length | Position | No |
| Forward | 5′-ctcatcctcagcaacaacca-3′ | 20 | 406 | 1375 |
| Probe | TET-5′-cctggatgattgtgccgagacact-3′- | 24 | 450 | 1376 |
| | TAMRA | | | |
| Reverse | 5′-tgtaggagaggtcgaggtcc-3′ | 20 | 477 | 1377 |

TABLE YC
Probe Name Ag2641

| | | | | SEQ |
|---|---|---|---|---|
| | | | Start | ID |
| Primers | Sequences | Length | Position | No |
| Forward | 5′-ctcatcctcagcaacaacca-3′ | 20 | 406 | 1378 |
| Probe | TET-5′-cctggatgattgtgccgagacact-3′- | 24 | 450 | 1379 |
| | TAMRA | | | |
| Reverse | 5′-tgtaggagaggtcgaggtcc-3′ | 20 | 477 | 1380 |

TABLE YD
CNS_neurodegeneration_v1.0

| Tissue Name | A |
|---|---|
| AD 1 Hippo | 55.5 |
| AD 2 Hippo | 79.0 |
| AD 3 Hippo | 35.8 |
| AD 4 Hippo | 63.3 |
| AD 5 hippo | 85.3 |
| AD 6 Hippo | 54.7 |
| Control 2 Hippo | 65.1 |
| Control 4 Hippo | 50.7 |
| Control (Path) 3 Hippo | 41.8 |
| AD 1 Temporal Ctx | 37.4 |
| AD 2 Temporal Ctx | 68.8 |
| AD 3 Temporal Ctx | 52.5 |
| AD 4 Temporal Ctx | 45.7 |
| AD 5 Inf Temporal Ctx | 63.7 |
| AD 5 Sup Temporal Ctx | 49.3 |
| AD 6 Inf Temporal Ctx | 58.6 |
| AD 6 Sup Temporal Ctx | 49.0 |
| Control 1 Temporal Ctx | 59.0 |
| Control 2 Temporal Ctx | 100.0 |
| Control 3 Temporal Ctx | 71.2 |
| Control 4 Temporal Ctx | 67.8 |
| Control (Path) 1 Temporal Ctx | 57.8 |
| Control (Path) 2 Temporal Ctx | 45.4 |
| Control (Path) 3 Temporal Ctx | 57.8 |
| Control (Path) 4 Temporal Ctx | 75.3 |
| AD 1 Occipital Ctx | 27.5 |
| AD 2 Occipital Ctx (Missing) | 2.0 |
| AD 3 Occipital Ctx | 36.3 |
| AD 4 Occipital Ctx | 32.5 |
| AD 5 Occipital Ctx | 37.4 |
| AD 6 Occipital Ctx | 49.7 |
| Control 1 Occipital Ctx | 55.1 |
| Control 2 Occipital Ctx | 92.0 |
| Control 3 Occipital Ctx | 57.8 |
| Control 4 Occipital Ctx | 46.3 |
| Control (Path) 1 Occipital Ctx | 69.7 |
| Control (Path) 2 Occipital Ctx | 63.3 |
| Control (Path) 3 Occipital Ctx | 51.8 |
| Control (Path) 4 Occipital Ctx | 82.9 |
| Control 1 Parietal Ctx | 39.5 |
| Control 2 Parietal Ctx | 54.3 |
| Control 3 Parietal Ctx | 67.4 |
| Control (Path) 1 Parietal Ctx | 84.7 |
| Control (Path) 2 Parietal Ctx | 56.6 |
| Control (Path) 3 Parietal Ctx | 47.0 |
| Control (Path) 4 Parietal Ctx | 79.6 |

Column A - Rel. Exp. (%) Ag2641, Run 206954924

TABLE YE
Panel 1.3D

| Tissue Name | A |
|---|---|
| Liver adenocarcinoma | 17.1 |
| Pancreas | 2.7 |
| Pancreatic ca. CAPAN 2 | 3.9 |
| Adrenal gland | 4.4 |
| Thyroid | 5.9 |
| Salivary gland | 7.0 |
| Pituitary gland | 16.5 |
| Brain (fetal) | 12.4 |
| Brain (whole) | 12.4 |
| Brain (amygdala) | 31.0 |
| Brain (cerebellum) | 11.2 |
| Brain (hippocampus) | 100.0 |
| Brain (substantia nigra) | 3.6 |
| Brain (thalamus) | 19.3 |
| Cerebral Cortex | 24.1 |
| Spinal cord | 5.9 |
| glio/astro U87-MG | 12.9 |
| glio/astro U-118-MG | 17.1 |
| astrocytoma SW1783 | 10.4 |
| neuro*; met SK-N-AS | 27.7 |
| astrocytoma SF-539 | 9.9 |
| astrocytoma SNB-75 | 19.9 |
| glioma SNB-19 | 11.2 |
| glioma U251 | 2.2 |
| glioma SF-295 | 11.2 |
| Heart (fetal) | 5.4 |
| Heart | 1.0 |
| Skeletal muscle (fetal) | 15.3 |
| Skeletal muscle | 1.5 |
| Bone marrow | 1.2 |
| Thymus | 2.2 |
| Spleen | 7.4 |
| Lymph node | 2.4 |
| Colorectal | 1.5 |
| Stomach | 7.1 |
| Small intestine | 12.8 |
| Colon ca. SW480 | 7.4 |
| Colon ca.* SW620(SW480 met) | 3.0 |
| Colon ca. HT29 | 2.5 |
| Colon ca. HCT-116 | 5.9 |
| Colon ca. CaCo-2 | 5.0 |
| Colon ca. tissue(ODO3866) | 6.7 |
| Colon ca. HCC-2998 | 23.2 |
| Gastric ca.* (liver met) NCI-N87 | 9.9 |
| Bladder | 1.2 |
| Trachea | 12.6 |
| Kidney | 1.2 |
| Kidney (fetal) | 4.0 |
| Renal ca. 786-0 | 1.9 |
| Renal ca. A498 | 15.2 |
| Renal ca. RXF 393 | 1.4 |
| Renal ca. ACHN | 2.9 |
| Renal ca. UO-31 | 3.4 |
| Renal ca. TK-10 | 1.6 |
| Liver | 2.9 |
| Liver (fetal) | 2.3 |
| Liver ca. (hepatoblast) HepG2 | 5.3 |
| Lung | 7.3 |
| Lung (fetal) | 5.1 |
| Lung ca. (small cell) LX-1 | 1.5 |
| Lung ca. (small cell) NCI-H69 | 9.2 |
| Lung ca. (s. cell var.) SHP-77 | 7.3 |
| Lung ca. (large cell)NCI-H460 | 3.3 |
| Lung ca. (non-sm. cell) A549 | 18.0 |
| Lung ca. (non-s. cell) NCI-H23 | 7.0 |
| Lung ca. (non-s. cell) HOP-62 | 7.3 |
| Lung ca. (non-s. cl) NCI-H522 | 4.4 |
| Lung ca. (squam.) SW 900 | 4.4 |
| Lung ca. (squam.) NCI-H596 | 0.6 |
| Mammary gland | 9.5 |
| Breast ca.* (pl. ef) MCF-7 | 6.3 |
| Breast ca.* (pl. ef) MDA-MB-231 | 48.3 |
| Breast ca.* (pl. ef) T47D | 0.7 |
| Breast ca. BT-549 | 13.9 |
| Breast ca. MDA-N | 8.5 |
| Ovary | 10.4 |
| Ovarian ca. OVCAR-3 | 7.2 |
| Ovarian ca. OVCAR-4 | 1.7 |
| Ovarian ca. OVCAR-5 | 3.6 |
| Ovarian ca. OVCAR-8 | 3.7 |
| Ovarian ca. IGROV-1 | 2.5 |
| Ovarian ca.* (ascites) SK-OV-3 | 3.5 |
| Uterus | 14.6 |
| Placenta | 3.4 |
| Prostate | 5.7 |
| Prostate ca.* (bone met)PC-3 | 5.6 |
| Testis | 10.2 |
| Melanoma Hs688(A).T | 5.3 |
| Melanoma* (met) Hs688(B).T | 3.1 |
| Melanoma UACC-62 | 2.0 |
| Melanoma M14 | 2.8 |
| Melanoma LOX IMVI | 15.0 |
| Melanoma* (met) SK-MEL-5 | 3.1 |
| Adipose | 2.0 |

Column A - Rel. Exp. (%) Ag2639, Run 157543474

TABLE YF
Panel 2D

| Tissue Name | A |
|---|---|
| Normal Colon | 43.5 |
| CC Well to Mod Diff (ODO3866) | 30.4 |
| CC Margin (ODO3866) | 18.8 |
| CC Gr.2 rectosigmoid (ODO3868) | 9.1 |
| CC Margin (ODO3868) | 3.6 |
| CC Mod Diff (ODO3920) | 22.8 |
| CC Margin (ODO3920) | 11.0 |
| CC Gr.2 ascend colon (ODO3921) | 40.1 |
| CC Margin (ODO3921) | 28.5 |
| CC from Partial Hepatectomy (ODO4309) Mets | 5.8 |
| Liver Margin (ODO4309) | 14.2 |
| Colon mets to lung (OD04451-01) | 15.8 |
| Lung Margin (OD04451-02) | 7.3 |
| Normal Prostate 6546-1 | 17.0 |
| Prostate Cancer (OD04410) | 53.2 |
| Prostate Margin (OD04410) | 35.4 |
| Prostate Cancer (OD04720-01) | 31.4 |
| Prostate Margin (OD04720-02) | 36.9 |
| Normal Lung 061010 | 29.1 |
| Lung Met to Muscle (ODO4286) | 8.7 |
| Muscle Margin (ODO4286) | 25.5 |
| Lung Malignant Cancer (OD03126) | 44.1 |
| Lung Margin (OD03126) | 23.3 |
| Lung Cancer (OD04404) | 33.2 |
| Lung Margin (OD04404) | 15.4 |
| Lung Cancer (OD04565) | 15.1 |
| Lung Margin (OD04565) | 6.0 |
| Lung Cancer (OD04237-01) | 14.8 |
| Lung Margin (OD04237-02) | 15.6 |
| Ocular Mel Met to Liver (ODO4310) | 13.5 |
| Liver Margin (ODO4310) | 17.6 |
| Melanoma Mets to Lung (OD04321) | 41.8 |
| Lung Margin (OD04321) | 28.3 |
| Normal Kidney | 43.2 |
| Kidney Ca, Nuclear grade 2 (OD04338) | 45.1 |
| Kidney Margin (OD04338) | 53.2 |
| Kidney Ca Nuclear grade 1/2 (OD04339) | 33.0 |
| Kidney Margin (OD04339) | 45.1 |
| Kidney Ca, Clear cell type (OD04340) | 15.3 |
| Kidney Margin (OD04340) | 36.3 |
| Kidney Ca, Nuclear grade 3 (OD04348) | 17.9 |
| Kidney Margin (OD04348) | 26.2 |
| Kidney Cancer (OD04622-01) | 42.9 |
| Kidney Margin (OD04622-03) | 15.3 |
| Kidney Cancer (OD04450-01) | 20.6 |
| Kidney Margin (OD04450-03) | 24.5 |
| Kidney Cancer 8120607 | 44.1 |
| Kidney Margin 8120608 | 34.4 |
| Kidney Cancer 8120613 | 39.0 |
| Kidney Margin 8120614 | 39.8 |
| Kidney Cancer 9010320 | 24.8 |
| Kidney Margin 9010321 | 41.5 |
| Normal Uterus | 12.7 |
| Uterus Cancer 064011 | 39.8 |
| Normal Thyroid | 23.3 |
| Thyroid Cancer 064010 | 21.3 |
| Thyroid Cancer A302152 | 11.3 |
| Thyroid Margin A302153 | 30.6 |
| Normal Breast | 45.1 |
| Breast Cancer (OD04566) | 16.6 |
| Breast Cancer (OD04590-01) | 81.8 |
| Breast Cancer Mets (OD04590-03) | 100.0 |
| Breast Cancer Metastasis (OD04655-05) | 72.7 |
| Breast Cancer 064006 | 16.3 |
| Breast Cancer 1024 | 44.8 |
| Breast Cancer 9100266 | 28.1 |
| Breast Margin 9100265 | 13.2 |
| Breast Cancer A209073 | 44.1 |
| Breast Margin A209073 | 24.1 |
| Normal Liver | 13.7 |
| Liver Cancer 064003 | 15.1 |
| Liver Cancer 1025 | 14.3 |
| Liver Cancer 1026 | 23.2 |
| Liver Cancer 6004-T | 16.2 |
| Liver Tissue 6004-N | 7.2 |
| Liver Cancer 6005-T | 36.9 |
| Liver Tissue 6005-N | 10.8 |
| Normal Bladder | 23.7 |
| Bladder Cancer 1023 | 19.9 |
| Bladder Cancer A302173 | 12.4 |
| Bladder Cancer (OD04718-01) | 38.2 |
| Bladder Normal Adjacent (OD04718-03) | 11.8 |
| Normal Ovary | 38.2 |
| Ovarian Cancer 064008 | 59.9 |
| Ovarian Cancer (OD04768-07) | 38.7 |
| Ovary Margin (OD04768-08) | 9.9 |
| Normal Stomach | 18.9 |
| Gastric Cancer 9060358 | 5.2 |
| Stomach Margin 9060359 | 25.5 |
| Gastric Cancer 9060395 | 28.5 |
| Stomach Margin 9060394 | 17.0 |
| Gastric Cancer 9060397 | 49.7 |
| Stomach Margin 9060396 | 22.8 |
| Gastric Cancer 064005 | 18.3 |

Column A - Rel. Exp. (%) Ag2640, Run 157543604

TABLE YG
Panel 4D

| Tissue Name | A |
|---|---|
| Secondary Th1 act | 72.7 |
| Secondary Th2 act | 75.3 |
| Secondary Tr1 act | 52.9 |
| Secondary Th1 rest | 36.6 |
| Secondary Th2 rest | 45.7 |
| Secondary Tr1 rest | 57.0 |
| Primary Th1 act | 34.9 |
| Primary Th2 act | 31.6 |
| Primary Tr1 act | 32.5 |
| Primary Th1 rest | 90.8 |
| Primary Th2 rest | 50.3 |
| Primary Tr1 rest | 25.9 |
| CD45RA CD4 lymphocyte act | 79.6 |
| CD45RO CD4 lymphocyte act | 53.6 |
| CD8 lymphocyte act | 49.3 |
| Secondary CD8 lymphocyte rest | 78.5 |
| Secondary CD8 lymphocyte act | 14.0 |
| CD4 lymphocyte none | 25.3 |
| 2ry Th1/Th2/Tr1_anti-CD95 CH11 | 26.1 |
| LAK cells rest | 26.1 |
| LAK cells IL-2 | 40.1 |
| LAK cells IL-2 + IL-12 | 47.0 |
| LAK cells IL-2 + IFN gamma | 49.7 |
| LAK cells IL-2 + IL-18 | 49.0 |
| LAK cells PMA/ionomycin | 14.8 |
| NK Cells IL-2 rest | 38.7 |
| Two Way MLR 3 day | 58.2 |
| Two Way MLR 5 day | 19.8 |
| Two Way MLR 7 day | 12.9 |
| PBMC rest | 23.2 |
| PBMC PWM | 89.5 |
| PBMC PHA-L | 35.4 |
| Ramos (B cell) none | 24.1 |
| Ramos (B cell) ionomycin | 93.3 |
| B lymphocytes PWM | 40.6 |
| B lymphocytes CD40L and IL-4 | 14.8 |
| EOL-1 dbcAMP | 100.0 |
| EOL-1 dbcAMP PMA/ionomycin | 81.2 |
| Dendritic cells none | 5.5 |
| Dendritic cells LPS | 0.0 |
| Dendritic cells anti-CD40 | 6.0 |
| Monocytes rest | 3.1 |
| Monocytes LPS | 4.6 |
| Macrophages rest | 11.0 |
| Macrophages LPS | 1.6 |
| HUVEC none | 63.7 |
| HUVEC starved | 48.6 |
| HUVEC IL-1beta | 0.0 |
| HUVEC IFN gamma | 96.6 |
| HUVEC TNF alpha + IFN gamma | 51.4 |
| HUVEC TNF alpha + IL4 | 93.3 |
| HUVEC IL-11 | 57.4 |
| Lung Microvascular EC none | 87.7 |
| Lung Microvascular EC TNFalpha + IL-1beta | 82.9 |
| Microvascular Dermal EC none | 71.2 |
| Microsvasular Dermal EC TNFalpha + IL-1beta | 43.5 |
| Bronchial epithelium TNFalpha + IL1beta | 95.9 |
| Small airway epithelium none | 25.7 |
| Small airway epithelium TNFalpha + IL-1beta | 82.4 |
| Coronery artery SMC rest | 53.6 |
| Coronery artery SMC TNFalpha + IL-1beta | 81.2 |
| Astrocytes rest | 57.8 |
| Astrocytes TNFalpha + IL-1beta | 41.5 |
| KU-812 (Basophil) rest | 25.7 |
| KU-812 (Basophil) PMA/ionomycin | 30.8 |
| CCD1106 (Keratinocytes) none | 54.3 |
| CCD1106 (Keratinocytes) TNFalpha + IL-1beta | 69.3 |
| Liver cirrhosis | 23.3 |
| Lupus kidney | 2.9 |
| NCI-H292 none | 36.1 |
| NCI-H292 IL-4 | 67.8 |
| NCI-H292 IL-9 | 62.0 |
| NCI-H292 IL-13 | 90.1 |
| NCI-H292 IFN gamma | 82.4 |
| HPAEC none | 49.3 |
| HPAEC TNF alpha + IL-1beta | 45.7 |
| Lung fibroblast none | 44.8 |
| Lung fibroblast TNF alpha + IL-1 beta | 35.6 |
| Lung fibroblast IL-4 | 69.3 |
| Lung fibroblast IL-9 | 43.8 |
| Lung fibroblast IL-13 | 45.1 |
| Lung fibroblast IFN gamma | 52.5 |
| Dermal fibroblast CCD1070 rest | 66.9 |
| Dermal fibroblast CCD1070 TNF alpha | 54.3 |
| Dermal fibroblast CCD1070 IL-1 beta | 71.2 |
| Dermal fibroblast IFN gamma | 84.1 |
| Dermal fibroblast IL-4 | 58.2 |
| IBD Colitis 2 | 3.1 |
| IBD Crohn's | 2.6 |
| Colon | 77.9 |
| Lung | 35.1 |
| Thymus | 41.2 |
| Kidney | 23.3 |

Column A - Rel. Exp. (%) Ag2641, Run 157543823

CNS_neurodegeneration_v1.0 Summary: Ag2641 This panel confirmed the expression of this gene at low levels in the brain in an independent group of individuals. This gene was found to be down-regulated in the temporal cortex of Alzheimer's disease patients patients when compared with non-demented controls (p=0.0003 when analyzed by Ancova, estimate of total cDNA loaded per well used asa covariate). Therefore, up-regulation of this gene or its protein product, or treatment with specific agonists for this receptor is of use in reversing the dementia, memory loss, and neuronal death associated with this disease.

Panel 1.3D Summary: Ag2639 Highest expression was seen in the hippocampus (CT=26.3). In addition, high to moderate levels of expression were seen in all regions of the CNS examined, including amygdala, hippocampus, substantia nigra, thalamus, and hypothalamus. This expression shows that therapeutic modulation of the expression or function of this gene is useful in the treatment of neurologic disorders, such as Alzheimer's disease, Parkinson's disease, schizophrenia, multiple sclerosis, stroke and epilepsy.

Panel 2D Summary: Ag2640 Highest expression was seen in a breast metastasis sample (CT=28.4), with moderate expression throughout this panel. This widespread expression demonstrates that this gene or gene product plays in cell growth and/or proliferation.

Panel 4D Summary: Ag2641 Highest expression was seen in an eosinophil cell line (CT=30). This gene was also expressed at moderate levels in a wide range of cell types of significance in the immune response in health and disease. These cells included members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression demonstrates that this gene product is involved in homeostatic processes for these and other cell types and tissues. This pattern was in agreement with the expression profile in Panel 2D and also shows a role for the gene or gene product in cell survival and proliferation. Therefore, modulation of the gene or its protein product with a functional therapeutic is useful for the alteration of functions associated with these cell types and for the improvement of the symptoms of patients suffering from autoimmune and inflammatory diseases such as asthma, allergies, inflammatory bowel disease, lupus erythematosus, psoriasis, rheumatoid arthritis, and osteoarthritis.

Z. CG5430804: Serpin

Expression of full-length physical clone CG54308-04 was assessed using the primer-probe sets Ag548 and Ag7880, described in Tables ZA and ZB. Results of the RTQ-PCR runs are shown in Tables ZC, ZD, ZE, ZF, and ZG.

TABLE ZA
Probe Name Ag548

| | | | | SEQ |
|---|---|---|---|---|
| | | | Start | ID |
| Primers | Sequences | Length | Position | No |
| Forward | 5′-tggagcagctcagaaaacatgt-3′ | 22 | 910 | 1381 |
| Probe | TET-5′-agaatcggtggtcctgtccttcccc-3′- | 25 | 936 | 1382 |
| | TAMRA | | | |
| Reverse | 5′-catagctgtcttccagggtgaac-3′ | 23 | 963 | 1383 |

TABLE ZB
Probe Name Ag7880

| | | | | SEQ |
|---|---|---|---|---|
| | | | Start | ID |
| Primers | Sequences | Length | Position | No |
| Forward | 5′-tccagaggctctgtcgtttt-3′ | 20 | 277 | 1384 |
| Probe | TET-5′-tccagagagctgtcagccagcacttt-3′- | 26 | 241 | 1385 |
| | TAMRA | | | |
| Reverse | 5′-gaacctgacccttgtctgaaa-3′ | 21 | 208 | 1386 |

TABLE ZC
General_screening_panel_v1.7

| Tissue Name | A |
|---|---|
| Adipose | 63.3 |
| HUVEC | 0.0 |
| Melanoma* Hs688(A).T | 0.0 |
| Melanoma* Hs688(B).T | 18.3 |
| Melanoma (met) SK-MEL-5 | 0.0 |
| Testis | 7.9 |
| Prostate ca. (bone met) PC-3 | 0.0 |
| Prostate ca. DU145 | 0.0 |
| Prostate pool | 0.0 |
| Uterus pool | 100.0 |
| Ovarian ca. OVCAR-3 | 0.0 |
| Ovarian ca. (ascites) SK-OV-3 | 0.0 |
| Ovarian ca. OVCAR-4 | 0.0 |
| Ovarian ca. OVCAR-5 | 8.1 |
| Ovarian ca. IGROV-1 | 0.0 |
| Ovarian ca. OVCAR-8 | 0.0 |
| Ovary | 0.0 |
| Breast ca. MCF-7 | 0.0 |
| Breast ca. MDA-MB-231 | 0.0 |
| Breast ca. BT-549 | 0.0 |
| Breast ca. T47D | 0.0 |
| Breast pool | 0.0 |
| Trachea | 12.1 |
| Lung | 0.0 |
| Fetal Lung | 0.0 |
| Lung ca. NCI-N417 | 0.0 |
| Lung ca. LX-1 | 0.0 |
| Lung ca. NCI-H146 | 0.0 |
| Lung ca. SHP-77 | 0.0 |
| Lung ca. NCI-H23 | 0.0 |
| Lung ca. NCI-H460 | 0.0 |
| Lung ca. HOP-62 | 0.0 |
| Lung ca. NCI-H522 | 0.0 |
| Lung ca. DMS-114 | 0.0 |
| Liver | 0.0 |
| Fetal Liver | 0.0 |
| Kidney pool | 0.1 |
| Fetal Kidney | 0.0 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 0.0 |
| Gastric ca. (liver met.) NCI-N87 | 0.0 |
| Stomach | 0.0 |
| Colon ca. SW-948 | 0.0 |
| Colon ca. SW480 | 0.0 |
| Colon ca. (SW480 met) SW620 | 0.0 |
| Colon ca. HT29 | 0.0 |
| Colon ca. HCT-116 | 0.0 |
| Colon cancer tissue | 0.0 |
| Colon ca. SW1116 | 0.0 |
| Colon ca. Colo-205 | 0.0 |
| Colon ca. SW-48 | 0.0 |
| Colon | 0.0 |
| Small Intestine | 0.0 |
| Fetal Heart | 0.0 |
| Heart | 0.0 |
| Lymph Node pool 1 | 0.0 |
| Lymph Node pool 2 | 0.0 |
| Fetal Skeletal Muscle | 6.7 |
| Skeletal Muscle pool | 0.0 |
| Skeletal Muscle | 0.0 |
| Spleen | 0.0 |
| Thymus | 7.5 |
| CNS cancer (glio/astro) SF-268 | 0.0 |
| CNS cancer (glio/astro) T98G | 0.0 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) | 0.0 |
| Brain (Cerebellum) | 0.0 |
| Brain (Fetal) | 0.0 |
| Brain (Hippocampus) | 0.0 |
| Cerebral Cortex pool | 0.0 |
| Brain (Substantia nigra) | 0.0 |
| Brain (Thalamus) | 0.0 |
| Brain (Whole) | 0.0 |
| Spinal Cord | 0.0 |
| Adrenal Gland | 0.0 |
| Pituitary Gland | 0.0 |
| Salivary Gland | 0.0 |
| Thyroid | 0.0 |
| Pancreatic ca. PANC-1 | 0.0 |
| Pancreas pool | 0.0 |

Column A - Rel. Exp. (%) Ag7880, Run 319066287

TABLE ZD
Panel 1.2

| Tissue Name | A |
|---|---|
| Endothelial cells | 0.0 |
| Heart (Fetal) | 0.0 |
| Pancreas | 0.0 |
| Pancreatic ca. CAPAN 2 | 0.0 |
| Adrenal Gland | 0.0 |
| Thyroid | 0.0 |
| Salivary gland | 0.0 |
| Pituitary gland | 0.0 |
| Brain (fetal) | 0.0 |
| Brain (whole) | 0.0 |
| Brain (amygdala) | 0.0 |
| Brain (cerebellum) | 0.0 |
| Brain (hippocampus) | 0.0 |
| Brain (thalamus) | 0.0 |
| Cerebral Cortex | 0.0 |
| Spinal cord | 0.0 |
| glio/astro U87-MG | 0.0 |
| glio/astro U-118-MG | 0.0 |
| astrocytoma SW1783 | 0.0 |
| neuro*; met SK-N-AS | 0.0 |
| astrocytoma SF-539 | 0.0 |
| astrocytoma SNB-75 | 0.0 |
| glioma SNB-19 | 0.0 |
| glioma U251 | 0.0 |
| glioma SF-295 | 0.0 |
| Heart | 0.0 |
| Skeletal Muscle | 0.0 |
| Bone marrow | 0.0 |
| Thymus | 100.0 |
| Spleen | 0.0 |
| Lymph node | 0.0 |
| Colorectal Tissue | 0.0 |
| Stomach | 0.0 |
| Small intestine | 0.0 |
| Colon ca. SW480 | 0.0 |
| Colon ca.* SW620 (SW480 met) | 0.0 |
| Colon ca. HT29 | 0.0 |
| Colon ca. HCT-116 | 0.0 |
| Colon ca. CaCo-2 | 0.0 |
| Colon ca. Tissue (ODO3866) | 0.0 |
| Colon ca. HCC-2998 | 0.0 |
| Gastric ca.* (liver met) NCI-N87 | 0.0 |
| Bladder | 0.0 |
| Trachea | 0.0 |
| Kidney | 0.0 |
| Kidney (fetal) | 0.0 |
| Renal ca. 786-0 | 0.0 |
| Renal ca. A498 | 0.0 |
| Renal ca. RXF 393 | 0.0 |
| Renal ca. ACHN | 0.0 |
| Renal ca. UO-31 | 0.0 |
| Renal ca. TK-10 | 0.0 |
| Liver | 0.0 |
| Liver (fetal) | 0.0 |
| Liver ca. (hepatoblast) HepG2 | 0.0 |
| Lung | 0.0 |
| Lung (fetal) | 0.0 |
| Lung ca. (small cell) LX-1 | 0.0 |
| Lung ca. (small cell) NCI-H69 | 0.0 |
| Lung ca. (s. cell var.) SHP-77 | 0.0 |
| Lung ca. (large cell)NCI-H460 | 0.0 |
| Lung ca. (non-sm. cell) A549 | 0.0 |
| Lung ca. (non-s. cell) NCI-H23 | 0.0 |
| Lung ca. (non-s. cell) HOP-62 | 0.0 |
| Lung ca. (non-s. cl) NCI-H522 | 0.0 |
| Lung ca. (squam.) SW 900 | 0.0 |
| Lung ca. (squam.) NCI-H596 | 0.0 |
| Mammary gland | 0.0 |
| Breast ca.* (pl. ef) MCF-7 | 0.0 |
| Breast ca.* (pl. ef) MDA-MB-231 | 0.0 |
| Breast ca.* (pl. ef) T47D | 0.0 |
| Breast ca. BT-549 | 0.0 |
| Breast ca. MDA-N | 0.0 |
| Ovary | 0.0 |
| Ovarian ca. OVCAR-3 | 0.0 |
| Ovarian ca. OVCAR-4 | 0.0 |
| Ovarian ca. OVCAR-5 | 0.0 |
| Ovarian ca. OVCAR-8 | 0.0 |
| Ovarian ca. IGROV-1 | 0.0 |
| Ovarian ca. (ascites) SK-OV-3 | 0.0 |
| Uterus | 0.0 |
| Placenta | 0.0 |
| Prostate | 0.0 |
| Prostate ca.* (bone met) PC-3 | 0.0 |
| Testis | 2.6 |
| Melanoma Hs688(A).T | 0.0 |
| Melanoma* (met) Hs688(B).T | 0.0 |
| Melanoma UACC-62 | 0.0 |
| Melanoma M14 | 0.0 |
| Melanoma LOX IMVI | 0.0 |
| Melanoma* (met) SK-MEL-5 | 0.0 |

Column A - Rel. Exp. (%) Ag548, Run 122671023

TABLE ZE
Panel 1.3D

| Tissue Name | A |
|---|---|
| Liver adenocarcinoma | 0.0 |
| Pancreas | 0.0 |
| Pancreatic ca. CAPAN 2 | 0.0 |
| Adrenal gland | 17.4 |
| Thyroid | 0.0 |
| Salivary gland | 0.0 |
| Pituitary gland | 0.0 |
| Brain (fetal) | 0.0 |
| Brain (whole) | 21.3 |
| Brain (amygdala) | 0.0 |
| Brain (cerebellum) | 0.0 |
| Brain (hippocampus) | 0.0 |
| Brain (substantia nigra) | 0.0 |
| Brain (thalamus) | 0.0 |
| Cerebral Cortex | 0.0 |
| Spinal cord | 0.0 |
| glio/astro U87-MG | 0.0 |
| glio/astro U-118-MG | 0.0 |
| astrocytoma SW1783 | 0.0 |
| neuro*; met SK-N-AS | 0.0 |
| astrocytoma SF-539 | 0.0 |
| astrocytoma SNB-75 | 0.0 |
| glioma SNB-19 | 0.0 |
| glioma U251 | 0.0 |
| glioma SF-295 | 0.0 |
| Heart (fetal) | 0.0 |
| Heart | 0.0 |
| Skeletal muscle (fetal) | 2.1 |
| Skeletal muscle | 0.0 |
| Bone marrow | 0.0 |
| Thymus | 100.0 |
| Spleen | 0.0 |
| Lymph node | 0.0 |
| Colorectal | 9.7 |
| Stomach | 0.0 |
| Small intestine | 0.0 |
| Colon ca. SW480 | 0.0 |
| Colon ca.* SW620(SW480 met) | 0.0 |
| Colon ca. HT29 | 0.0 |
| Colon ca. HCT-116 | 0.0 |
| Colon ca. CaCo-2 | 0.0 |
| Colon ca. tissue(ODO3866) | 0.0 |
| Colon ca. HCC-2998 | 0.0 |
| Gastric ca.* (liver met) NCI-N87 | 0.0 |
| Bladder | 0.0 |
| Trachea | 0.0 |
| Kidney | 0.0 |
| Kidney (fetal) | 0.0 |
| Renal ca. 786-0 | 2.9 |
| Renal ca. A498 | 0.0 |
| Renal ca. RXF 393 | 0.0 |
| Renal ca. ACHN | 0.0 |
| Renal ca. UO-31 | 0.0 |
| Renal ca. TK-10 | 0.0 |
| Liver | 0.0 |
| Liver (fetal) | 0.0 |
| Liver ca. (hepatoblast) HepG2 | 0.0 |
| Lung | 11.3 |
| Lung (fetal) | 0.0 |
| Lung ca. (small cell) LX-1 | 0.0 |
| Lung ca. (small cell) NCI-H69 | 0.0 |
| Lung ca. (s. cell var.) SHP-77 | 0.0 |
| Lung ca. (large cell)NCI-H460 | 0.0 |
| Lung ca. (non-sm. cell) A549 | 0.0 |
| Lung ca. (non-s. cell) NCI-H23 | 0.0 |
| Lung ca. (non-s. cell) HOP-62 | 0.0 |
| Lung ca. (non-s. cl) NCI-H522 | 0.0 |
| Lung ca. (squam.) SW 900 | 0.0 |
| Lung ca. (squam.) NCI-H596 | 14.0 |
| Mammary gland | 23.7 |
| Breast ca.* (pl. ef) MCF-7 | 0.0 |
| Breast ca.* (pl. ef) MDA-MB-231 | 0.0 |
| Breast ca.* (pl. ef) T47D | 11.5 |
| Breast ca. BT-549 | 0.0 |
| Breast ca. MDA-N | 22.8 |
| Ovary | 0.0 |
| Ovarian ca. OVCAR-3 | 0.0 |
| Ovarian ca. OVCAR-4 | 0.0 |
| Ovarian ca. OVCAR-5 | 0.0 |
| Ovarian ca. OVCAR-8 | 0.0 |
| Ovarian ca. IGROV-1 | 0.0 |
| Ovarian ca.* (ascites) SK-OV-3 | 0.0 |
| Uterus | 0.0 |
| Placenta | 0.0 |
| Prostate | 5.3 |
| Prostate ca.* (bone met)PC-3 | 12.8 |
| Testis | 22.1 |
| Melanoma Hs688(A).T | 11.0 |
| Melanoma* (met) Hs688(B).T | 10.6 |
| Melanoma UACC-62 | 0.0 |
| Melanoma M14 | 0.0 |
| Melanoma LOX IMVI | 0.0 |
| Melanoma* (met) SK-MEL-5 | 0.0 |
| Adipose | 0.0 |

Column A - Rel. Exp. (%) Ag548, Run 167658585

TABLE ZF
Panel 2D

| Tissue Name | A | B |
|---|---|---|
| Normal Colon | 0.0 | 0.0 |
| CC Well to Mod Diff (ODO3866) | 5.6 | 4.6 |
| CC Margin (ODO3866) | 3.3 | 3.1 |
| CC Gr.2 rectosigmoid (ODO3868) | 0.0 | 0.0 |
| CC Margin (ODO3868) | 0.0 | 0.0 |
| CC Mod Diff (ODO3920) | 0.0 | 0.0 |
| CC Margin (ODO3920) | 0.0 | 0.0 |
| CC Gr.2 ascend colon (ODO3921) | 0.0 | 0.0 |
| CC Margin (ODO3921) | 6.3 | 0.0 |
| CC from Partial Hepatectomy | 0.0 | 0.0 |
| (ODO4309) Mets | | |
| Liver Margin (ODO4309) | 0.0 | 0.0 |
| Colon mets to lung (OD04451-01) | 4.6 | 2.4 |
| Lung Margin (OD04451-02) | 2.9 | 0.0 |
| Normal Prostate 6546-1 | 2.5 | 6.1 |
| Prostate Cancer (OD04410) | 0.0 | 0.0 |
| Prostate Margin (OD04410) | 3.1 | 0.0 |
| Prostate Cancer (OD04720-01) | 4.5 | 0.0 |
| Prostate Margin (OD04720-02) | 0.0 | 0.0 |
| Normal Lung 061010 | 6.6 | 0.0 |
| Lung Met to Muscle (ODO4286) | 12.9 | 3.7 |
| Muscle Margin (ODO4286) | 2.8 | 0.0 |
| Lung Malignant Cancer (OD03126) | 2.8 | 0.0 |
| Lung Margin (OD03126) | 3.0 | 0.0 |
| Lung Cancer (OD04404) | 0.0 | 0.0 |
| Lung Margin (OD04404) | 0.0 | 0.0 |
| Lung Cancer (OD04565) | 17.3 | 6.1 |
| Lung Margin (OD04565) | 1.4 | 0.0 |
| Lung Cancer (OD04237-01) | 0.0 | 0.0 |
| Lung Margin (OD04237-02) | 2.9 | 0.0 |
| Ocular Mel Met to Liver (ODO4310) | 5.1 | 0.0 |
| Liver Margin (ODO4310) | 0.0 | 2.0 |
| Melanoma Mets to Lung (OD04321) | 0.0 | 0.0 |
| Lung Margin (OD04321) | 0.0 | 0.0 |
| Normal Kidney | 0.0 | 0.0 |
| Kidney Ca, Nuclear grade 2 (OD04338) | 0.0 | 0.0 |
| Kidney Margin (OD04338) | 0.0 | 1.0 |
| Kidney Ca Nuclear grade 1/2 (OD04339) | 0.0 | 0.0 |
| Kidney Margin (OD04339) | 0.0 | 0.0 |
| Kidney Ca, Clear cell type (OD04340) | 0.0 | 2.8 |
| Kidney Margin (OD04340) | 0.0 | 0.0 |
| Kidney Ca, Nuclear grade 3 (OD04348) | 0.0 | 0.0 |
| Kidney Margin (OD04348) | 0.0 | 0.0 |
| Kidney Cancer (OD04622-01) | 0.0 | 0.0 |
| Kidney Margin (OD04622-03) | 4.6 | 3.8 |
| Kidney Cancer (OD04450-01) | 0.0 | 4.5 |
| Kidney Margin (OD04450-03) | 0.0 | 2.3 |
| Kidney Cancer 8120607 | 0.0 | 0.0 |
| Kidney Margin 8120608 | 0.0 | 0.0 |
| Kidney Cancer 8120613 | 1.6 | 2.6 |
| Kidney Margin 8120614 | 0.0 | 0.0 |
| Kidney Cancer 9010320 | 0.0 | 0.0 |
| Kidney Margin 9010321 | 0.0 | 0.0 |
| Normal Uterus | 0.0 | 8.8 |
| Uterus Cancer 064011 | 0.0 | 0.0 |
| Normal Thyroid | 2.8 | 2.7 |
| Thyroid Cancer 064010 | 0.0 | 4.5 |
| Thyroid Cancer A302152 | 1.8 | 0.0 |
| Thyroid Margin A302153 | 0.0 | 0.0 |
| Normal Breast | 2.5 | 0.0 |
| Breast Cancer (OD04566) | 2.3 | 0.0 |
| Breast Cancer (OD04590-01) | 0.0 | 0.0 |
| Breast Cancer Mets (OD04590-03) | 0.0 | 0.0 |
| Breast Cancer Metastasis (OD04655-05) | 0.0 | 0.0 |
| Breast Cancer 064006 | 0.0 | 0.0 |
| Breast Cancer 1024 | 5.8 | 0.0 |
| Breast Cancer 9100266 | 0.0 | 0.0 |
| Breast Margin 9100265 | 0.0 | 0.0 |
| Breast Cancer A209073 | 9.5 | 0.0 |
| Breast Margin A209073 | 0.0 | 0.0 |
| Normal Liver | 0.0 | 3.0 |
| Liver Cancer 064003 | 0.0 | 2.7 |
| Liver Cancer 1025 | 0.0 | 0.0 |
| Liver Cancer 1026 | 0.0 | 0.0 |
| Liver Cancer 6004-T | 0.0 | 0.0 |
| Liver Tissue 6004-N | 8.4 | 4.5 |
| Liver Cancer 6005-T | 0.0 | 0.0 |
| Liver Tissue 6005-N | 0.0 | 0.0 |
| Normal Bladder | 0.0 | 2.8 |
| Bladder Cancer 1023 | 0.0 | 5.0 |
| Bladder Cancer A302173 | 100.0 | 100.0 |
| Bladder Cancer (OD04718-01) | 0.0 | 0.0 |
| Bladder Normal Adjacent (OD04718-03) | 0.0 | 4.4 |
| Normal Ovary | 0.0 | 0.0 |
| Ovarian Cancer 064008 | 0.0 | 0.0 |
| Ovarian Cancer (OD04768-07) | 0.0 | 0.0 |
| Ovary Margin (OD04768-08) | 0.0 | 0.0 |
| Normal Stomach | 0.0 | 0.0 |
| Gastric Cancer 9060358 | 0.0 | 0.0 |
| Stomach Margin 9060359 | 0.0 | 0.0 |
| Gastric Cancer 9060395 | 0.0 | 0.0 |
| Stomach Margin 9060394 | 0.0 | 0.0 |
| Gastric Cancer 9060397 | 0.0 | 0.0 |
| Stomach Margin 9060396 | 0.0 | 0.0 |
| Gastric Cancer 064005 | 1.5 | 6.7 |

Column A - Rel. Exp. (%) Ag548, Run 145364354
Column B - Rel. Exp. (%) Ag548, Run 145506152

TABLE ZG
Panel 4D

| Tissue Name | A | B | C |
|---|---|---|---|
| Secondary Th1 act | 0.0 | 3.1 | 0.0 |
| Secondary Th2 act | 0.0 | 0.0 | 4.9 |
| Secondary Tr1 act | 0.0 | 0.0 | 0.0 |
| Secondary Th1 rest | 0.0 | 0.0 | 0.0 |
| Secondary Th2 rest | 0.0 | 0.0 | 0.0 |
| Secondary Tr1 rest | 0.0 | 7.1 | 0.0 |
| Primary Th1 act | 0.0 | 0.0 | 0.0 |
| Primary Th2 act | 0.0 | 1.1 | 0.0 |
| Primary Tr1 act | 0.0 | 0.0 | 0.0 |
| Primary Th1 rest | 0.0 | 0.0 | 0.0 |
| Primary Th2 rest | 0.0 | 0.0 | 0.0 |
| Primary Tr1 rest | 0.0 | 0.0 | 5.3 |
| CD45RA CD4 lymphocyte act | 0.0 | 0.0 | 0.0 |
| CD45RO CD4 lymphocyte act | 0.0 | 0.0 | 0.0 |
| CD8 lymphocyte act | 0.0 | 0.0 | 0.0 |
| Secondary CD8 lymphocyte rest | 0.0 | 0.0 | 0.0 |
| Secondary CD8 lymphocyte act | 0.0 | 0.0 | 0.0 |
| CD4 lymphocyte none | 0.0 | 0.0 | 0.0 |
| 2ry Th1/Th2/Tr1_anti-CD95 CH11 | 0.0 | 0.0 | 0.0 |
| LAK cells rest | 0.0 | 0.0 | 0.0 |
| LAK cells IL-2 | 5.8 | 0.0 | 0.0 |
| LAK cells IL-2 + IL-12 | 0.0 | 0.0 | 0.0 |
| LAK cells IL-2 + IFN gamma | 0.0 | 0.0 | 0.0 |
| LAK cells IL-2 + IL-18 | 0.0 | 0.0 | 0.0 |
| LAK cells PMA/ionomycin | 0.0 | 0.0 | 0.0 |
| NK Cells IL-2 rest | 0.0 | 0.0 | 0.0 |
| Two Way MLR 3 day | 0.0 | 3.3 | 0.0 |
| Two Way MLR 5 day | 0.0 | 0.0 | 0.0 |
| Two Way MLR 7 day | 0.0 | 0.0 | 0.0 |
| PBMC rest | 0.0 | 0.0 | 0.0 |
| PBMC PWM | 0.0 | 0.0 | 0.0 |
| PBMC PHA-L | 0.0 | 0.0 | 0.0 |
| Ramos (B cell) none | 0.0 | 0.0 | 0.0 |
| Ramos (B cell) ionomycin | 0.0 | 0.0 | 0.0 |
| B lymphocytes PWM | 0.0 | 0.0 | 0.0 |
| B lymphocytes CD40L and IL-4 | 0.0 | 0.0 | 0.0 |
| EOL-1 dbcAMP | 0.0 | 0.0 | 0.0 |
| EOL-1 dbcAMP PMA/ionomycin | 0.0 | 0.0 | 0.0 |
| Dendritic cells none | 3.5 | 0.0 | 0.0 |
| Dendritic cells LPS | 0.0 | 0.0 | 5.4 |
| Dendritic cells anti-CD40 | 0.0 | 0.0 | 0.0 |
| Monocytes rest | 0.0 | 0.0 | 0.0 |
| Monocytes LPS | 0.0 | 0.0 | 0.0 |
| Macrophages rest | 4.5 | 0.0 | 0.0 |
| Macrophages LPS | 2.4 | 8.0 | 0.0 |
| HUVEC none | 0.0 | 0.0 | 0.0 |
| HUVEC starved | 0.0 | 0.0 | 0.0 |
| HUVEC IL-1beta | 0.0 | 0.0 | 0.0 |
| HUVEC IFN gamma | 0.0 | 0.0 | 0.0 |
| HUVEC TNF alpha + IFN gamma | 0.0 | 0.0 | 0.0 |
| HUVEC TNF alpha + IL4 | 0.0 | 0.0 | 0.0 |
| HUVEC IL-11 | 0.0 | 0.0 | 0.0 |
| Lung Microvascular EC none | 0.0 | 2.9 | 0.0 |
| Lung Microvascular EC TNFalpha + | 0.0 | 0.0 | 0.0 |
| IL-1beta | | | |
| Microvascular Dermal EC none | 0.0 | 0.0 | 0.0 |
| Microsvasular Dermal EC | 0.0 | 0.0 | 0.0 |
| TNFalpha + IL-1beta | | | |
| Bronchial epithelium TNFalpha + | 6.5 | 0.0 | 0.0 |
| IL1beta | | | |
| Small airway epithelium none | 13.4 | 13.6 | 8.8 |
| Small airway epithelium TNFalpha + | 100.0 | 73.2 | 95.9 |
| IL-1beta | | | |
| Coronery artery SMC rest | 0.0 | 0.0 | 0.0 |
| Coronery artery SMC TNFalpha + | 0.0 | 0.0 | 0.0 |
| IL-1beta | | | |
| Astrocytes rest | 0.0 | 0.0 | 0.0 |
| Astrocytes TNFalpha + IL-1beta | 0.0 | 0.0 | 5.5 |
| KU-812 (Basophil) rest | 0.0 | 0.0 | 0.0 |
| KU-812 (Basophil) PMA/ionomycin | 0.0 | 0.0 | 0.0 |
| CCD1106 (Keratinocytes) none | 0.0 | 3.4 | 0.0 |
| CCD1106 (Keratinocytes) TNFalpha + | 4.3 | 2.4 | 0.0 |
| IL-1beta | | | |
| Liver cirrhosis | 22.5 | 21.0 | 4.1 |
| Lupus kidney | 0.0 | 0.0 | 0.0 |
| NCI-H292 none | 6.2 | 7.7 | 22.7 |
| NCI-H292 IL-4 | 4.2 | 0.0 | 23.0 |
| NCI-H292 IL-9 | 3.7 | 11.4 | 9.0 |
| NCI-H292 IL-13 | 17.0 | 13.2 | 17.1 |
| NCI-H292 IFN gamma | 0.0 | 2.3 | 13.0 |
| HPAEC none | 0.0 | 0.0 | 0.0 |
| HPAEC TNF alpha + IL-1 beta | 8.4 | 0.0 | 0.0 |
| Lung fibroblast none | 6.8 | 0.0 | 0.0 |
| Lung fibroblast TNF alpha + | 0.0 | 0.0 | 6.6 |
| IL-1 beta | | | |
| Lung fibroblast IL-4 | 0.0 | 0.0 | 0.0 |
| Lung fibroblast IL-9 | 0.0 | 0.0 | 0.0 |
| Lung fibroblast IL-13 | 0.0 | 0.0 | 0.0 |
| Lung fibroblast IFN gamma | 0.0 | 0.0 | 0.0 |
| Dermal fibroblast CCD1070 rest | 0.0 | 2.8 | 0.0 |
| Dermal fibroblast CCD1070 TNF alpha | 0.0 | 2.0 | 0.0 |
| Dermal fibroblast CCD1070 IL-1 beta | 0.0 | 0.0 | 6.3 |
| Dermal fibroblast IFN gamma | 0.0 | 0.0 | 0.0 |
| Dermal fibroblast IL-4 | 0.0 | 0.0 | 0.0 |
| IBD Colitis 2 | 3.5 | 2.5 | 19.9 |
| IBD Crohn's | 0.0 | 13.3 | 0.0 |
| Colon | 0.0 | 0.0 | 0.0 |
| Lung | 0.0 | 0.0 | 0.0 |
| Thymus | 0.0 | 0.0 | 0.0 |
| Kidney | 87.1 | 100.0 | 100.0 |

Column A - Rel. Exp. (%) Ag548, Run 145909057
Column B - Rel. Exp. (%) Ag548, Run 145928351
Column C - Rel. Exp. (%) Ag548, Run 164886875

General_screening₁₃ panel_v1.7 Summary: Ag7880 Prominent expressin of this gene was seen in uterus and adipose (CTs=30).

Panel 1.2 Summary: Ag548 This gene was expressed at a very low level in this panel. The highest level of expression was seen in thymus (CT=34.33). Thus, expression of this gene is useful as a marker to differentiate thymus tissues from other samples on this panel.

Panel 1.3D Summary: Ag548 This gene was expressed at a very low level in this panel. The highest level of expression was seen in thymus (CT=34.33). Thus, expression of this gene is useful as a marker to differentiate thymus tissues from other samples on this panel.

Panel 2D Summary: Ag548 The highest level of expression in this panel was detected in a bladder cancer sample (CTs=30). Thus, expression of the gene or its protein product is useful as a marker to differentiate between normal and cancerous bladder tissue and therapeutic modulation of the gene product using antibodies , chimeric molecules or small molecule inhibitors is effective in the treatment of bladder cancer.

Panel 4D Summary: Ag548 This transcript was induced in small airway epithelium activated with TNFalpha+IL-1beta and was present in normal kidney. The transcript encodes a putative serpin like molecule (serine proteinase inhibitor). Serpins participate in multiple biological processes and mutations that alter serpin function can result in pulmonary dysfunction including asthma and emphysema. Therapies designed with the protein encoded for by this transcript are important for the treatment of lung disorders such as asthma and emphysema.

AA. CG54764-02: CALGIZZARIN

Expression of gene CG54764-02 was assessed using the primer-probe sets Ag1212 and Ag1528, described in Tables AAA and AAB. Results of the RTQ-PCR runs are shown in Tables AAC, AAD, AAE, AAF, and AAG.

TABLE AAA
Probe Name Ag1212

| | | | | SEQ |
|---|---|---|---|---|
| | | | Start | ID |
| Primers | Sequences | Length | Position | No |
| Forward | 5′-gaccataacagctgcaaactct-3′ | 22 | 103 | 1387 |
| Probe | TET-5′-ttcatgaacactgtactggttgcctt-3′- | 26 | 145 | 1388 |
| | TAMRA | | | |
| Reverse | 5′-agcccttctggttctttgtg-3′ | 20 | 171 | 1389 |

TABLE AAB
Probe Name Ag1528

| | | | | SEQ |
|---|---|---|---|---|
| | | | Start | ID |
| Primers | Sequences | Length | Position | No |
| Forward | 5′-gaccataacagctgcaaactct-3′ | 22 | 103 | 1390 |
| Probe | TET-5′-tgaacactgtactggttgccttcaca-3′- | 26 | 149 | 1391 |
| | TAMRA | | | |
| Reverse | 5′-ccagagcccttctggttct-3′ | 19 | 176 | 1392 |

TABLE AAC
CNS_neurodegeneration_v1.0

| Tissue Name | A |
|---|---|
| AD 1 Hippo | 15.9 |
| AD 2 Hippo | 18.7 |
| AD 3 Hippo | 10.6 |
| AD 4 Hippo | 24.5 |
| AD 5 Hippo | 92.0 |
| AD 6 Hippo | 13.4 |
| Control 2 Hippo | 6.8 |
| Control 4 Hippo | 8.7 |
| Control (Path) 3 Hippo | 6.6 |
| AD 1 Temporal Ctx | 30.8 |
| AD 2 Temporal Ctx | 39.8 |
| AD 3 Temporal Ctx | 40.9 |
| AD 4 Temporal Ctx | 54.0 |
| AD 5 Inf Temporal Ctx | 51.8 |
| AD 5 Sup Temporal Ctx | 33.0 |
| AD 6 Inf Temporal Ctx | 27.2 |
| AD 6 Sup Temporal Ctx | 57.8 |
| Control 1 Temporal Ctx | 21.6 |
| Control 2 Temporal Ctx | 9.2 |
| Control 3 Temporal Ctx | 44.4 |
| Control 3 Temporal Ctx | 19.5 |
| Control (Path) 1 Temporal Ctx | 54.3 |
| Control (Path) 2 Temporal Ctx | 74.2 |
| Control (Path) 3 Temporal Ctx | 9.1 |
| Control (Path) 4 Temporal Ctx | 75.3 |
| AD 1 Occipital Ctx | 31.0 |
| AD 2 Occipital Ctx (Missing) | 2.8 |
| AD 3 Occipital Ctx | 20.6 |
| AD 4 Occipital Ctx | 55.1 |
| AD 5 Occipital Ctx | 16.5 |
| AD 6 Occipital Ctx | 27.5 |
| Control 1 Occipital Ctx | 7.8 |
| Control 2 Occipital Ctx | 17.1 |
| Control 3 Occipital Ctx | 36.6 |
| Control 4 Occipital Ctx | 8.4 |
| Control (Path) 1 Occipital Ctx | 68.8 |
| Control (Path) 2 Occipital Ctx | 43.5 |
| Control (Path) 3 Occipital Ctx | 0.3 |
| Control (Path) 4 Occipital Ctx | 100.0 |
| Control 1 Parietal Ctx | 23.2 |
| Control 2 Parietal Ctx | 38.2 |
| Control 3 Parietal Ctx | 17.9 |
| Control (Path) 1 Parietal Ctx | 42.9 |
| Control (Path) 2 Parietal Ctx | 50.7 |
| Control (Path) 3 Parietal Ctx | 5.7 |
| Control (Path) 4 Parietal Ctx | 84.7 |

Column A - Rel. Exp. (%) Ag1212, Run 206992259

TABLE AAD
Panel 1.3D

| Tissue Name | A | B | C |
|---|---|---|---|
| Liver adenocarcinoma | 17.1 | 16.2 | 11.8 |
| Pancreas | 0.0 | 0.0 | 0.0 |
| Pancreatic ca. CAPAN 2 | 3.9 | 0.6 | 2.3 |
| Adrenal gland | 0.0 | 0.0 | 0.0 |
| Thyroid | 0.0 | 0.0 | 0.0 |
| Salivary gland | 0.0 | 0.0 | 1.4 |
| Pituitary gland | 9.8 | 11.1 | 4.5 |
| Brain (fetal) | 100.0 | 32.5 | 25.9 |
| Brain (whole) | 80.1 | 23.7 | 36.6 |
| Brain (amygdala) | 29.5 | 27.0 | 46.3 |
| Brain (cerebellum) | 99.3 | 17.3 | 18.9 |
| Brain (hippocampus) | 64.6 | 32.1 | 100.0 |
| Brain (substantia nigra) | 10.2 | 0.0 | 4.0 |
| Brain (thalamus) | 27.2 | 17.0 | 24.7 |
| Cerebral Cortex | 30.8 | 100.0 | 66.0 |
| Spinal cord | 2.6 | 0.6 | 1.2 |
| glio/astro U87-MG | 38.4 | 49.3 | 17.6 |
| glio/astro U-118-MG | 12.9 | 8.8 | 3.7 |
| astrocytoma SW1783 | 8.4 | 7.3 | 9.7 |
| neuro*; met SK-N-AS | 1.4 | 4.8 | 5.6 |
| astrocytoma SF-539 | 10.2 | 7.4 | 8.0 |
| astrocytoma SNB-75 | 9.0 | 14.6 | 9.0 |
| glioma SNB-19 | 0.0 | 0.8 | 1.5 |
| glioma U251 | 13.7 | 7.1 | 4.4 |
| glioma SF-295 | 2.2 | 0.0 | 0.0 |
| Heart (fetal) | 0.0 | 0.0 | 0.0 |
| Heart | 1.6 | 0.0 | 0.0 |
| Skeletal muscle (fetal) | 3.1 | 1.4 | 5.3 |
| Skeletal muscle | 0.0 | 0.0 | 0.0 |
| Bone marrow | 0.0 | 0.0 | 0.0 |
| Thymus | 0.0 | 0.0 | 0.0 |
| Spleen | 0.0 | 0.0 | 0.0 |
| Lymph node | 0.0 | 0.0 | 0.0 |
| Colorectal | 0.0 | 0.9 | 2.7 |
| Stomach | 0.0 | 0.5 | 1.3 |
| Small intestine | 7.2 | 0.0 | 1.4 |
| Colon ca. SW480 | 0.0 | 0.0 | 1.2 |
| Colon ca.* SW620(SW480 met) | 0.0 | 1.1 | 0.0 |
| Colon ca. HT29 | 0.0 | 1.1 | 0.0 |
| Colon ca. HCT-116 | 0.0 | 0.0 | 0.0 |
| Colon ca. CaCo-2 | 0.0 | 0.0 | 0.0 |
| Colon ca. tissue(ODO3866) | 0.0 | 2.8 | 1.2 |
| Colon ca. HCC-2998 | 0.0 | 0.0 | 0.0 |
| Gastric ca.* (liver met) NCI-N87 | 0.0 | 0.0 | 0.0 |
| Bladder | 4.6 | 4.4 | 2.7 |
| Trachea | 0.0 | 3.5 | 1.2 |
| Kidney | 0.0 | 0.0 | 0.0 |
| Kidney (fetal) | 0.0 | 1.2 | 0.0 |
| Renal ca. 786-0 | 0.0 | 0.0 | 0.0 |
| Renal ca. A498 | 6.7 | 12.2 | 6.7 |
| Renal ca. RXF 393 | 0.0 | 0.0 | 0.0 |
| Renal ca. ACHN | 0.0 | 0.0 | 0.0 |
| Renal ca. UO-31 | 7.3 | 6.6 | 2.1 |
| Renal ca. TK-10 | 0.0 | 0.0 | 0.0 |
| Liver | 3.1 | 1.0 | 0.0 |
| Liver (fetal) | 0.0 | 0.0 | 0.0 |
| Liver ca. (hepatoblast) HepG2 | 0.0 | 0.0 | 0.0 |
| Lung | 0.0 | 0.0 | 0.0 |
| Lung (fetal) | 0.0 | 0.0 | 0.0 |
| Lung ca. (small cell) LX-1 | 0.0 | 0.0 | 0.0 |
| Lung ca. (small cell) NCI-H69 | 1.8 | 9.4 | 6.1 |
| Lung ca. (s. cell var.) SHP-77 | 2.1 | 0.0 | 0.0 |
| Lung ca. (large cell)NCI-H460 | 0.0 | 0.0 | 0.0 |
| Lung ca. (non-sm. cell) A549 | 0.0 | 3.5 | 4.2 |
| Lung ca. (non-s. cell) NCI-H23 | 2.5 | 0.0 | 0.0 |
| Lung ca. (non-s. cell) HOP-62 | 15.4 | 13.7 | 4.2 |
| Lung ca. (non-s. cl) NCI-H522 | 3.6 | 12.5 | 9.3 |
| Lung ca. (squam.) SW 900 | 0.0 | 0.0 | 0.0 |
| Lung ca. (squam.) NCI-H596 | 21.3 | 20.9 | 21.0 |
| Mammary gland | 5.1 | 7.4 | 0.0 |
| Breast ca.* (pl. ef) MCF-7 | 0.0 | 0.0 | 0.0 |
| Breast ca.* (pl. ef) MDA-MB-231 | 0.0 | 0.0 | 0.0 |
| Breast ca.* (pl. ef) T47D | 0.0 | 2.0 | 0.0 |
| Breast ca. BT-549 | 0.0 | 0.8 | 1.2 |
| Breast ca. MDA-N | 0.0 | 32.1 | 18.0 |
| Ovary | 0.0 | 0.0 | 0.0 |
| Ovarian ca. OVCAR-3 | 40.1 | 37.4 | 18.6 |
| Ovarian ca. OVCAR-4 | 0.0 | 0.0 | 2.6 |
| Ovarian ca. OVCAR-5 | 5.4 | 0.0 | 1.3 |
| Ovarian ca. OVCAR-8 | 4.4 | 1.2 | 4.3 |
| Ovarian ca. IGROV-1 | 0.0 | 1.1 | 0.0 |
| Ovarian ca.* (ascites) SK-OV-3 | 0.0 | 15.5 | 8.1 |
| Uterus | 0.0 | 0.0 | 0.0 |
| Placenta | 0.0 | 2.2 | 0.0 |
| Prostate | 2.6 | 0.0 | 0.0 |
| Prostate ca.* (bone met)PC-3 | 10.1 | 1.1 | 0.7 |
| Testis | 3.0 | 1.4 | 4.1 |
| Melanoma Hs688(A).T | 6.2 | 37.6 | 12.8 |
| Melanoma* (met) Hs688(B).T | 18.8 | 54.7 | 14.0 |
| Melanoma UACC-62 | 10.3 | 1.0 | 1.2 |
| Melanoma M14 | 85.3 | 9.6 | 26.4 |
| Melanoma LOX IMVI | 23.2 | 34.9 | 57.0 |
| Melanoma* (met) SK-MEL-5 | 9.6 | 0.0 | 14.6 |
| Adipose | 2.0 | 3.7 | 2.4 |

Column A - Rel. Exp. (%) Ag1212, Run 165519986
Column B - Rel. Exp. (%) Ag1528, Run 147327845
Column C - Rel. Exp. (%) Ag1528, Run 148390885

TABLE AAE
Panel 2D

| Tissue Name | A | B |
|---|---|---|
| Normal Colon | 7.4 | 8.5 |
| CC Well to Mod Diff (ODO3866) | 3.9 | 2.3 |
| CC Margin (ODO3866) | 0.0 | 2.7 |
| CC Gr.2 rectosigmoid (ODO3868) | 0.0 | 4.7 |
| CC Margin (ODO3868) | 0.0 | 0.0 |
| CC Mod Diff (ODO3920) | 0.0 | 0.0 |
| CC Margin (ODO3920) | 0.0 | 1.2 |
| CC Gr.2 ascend colon (ODO3921) | 0.0 | 0.0 |
| CC Margin (ODO3921) | 1.9 | 1.3 |
| CC from Partial Hepatectomy | 0.0 | 2.4 |
| (ODO4309) Mets | | |
| Liver Margin (ODO4309) | 0.0 | 1.3 |
| Colon mets to lung (OD04451-01) | 0.0 | 1.1 |
| Lung Margin (OD04451-02) | 0.0 | 0.0 |
| Normal Prostate 6546-1 | 0.0 | 1.2 |
| Prostate Cancer (OD04410) | 0.0 | 0.0 |
| Prostate Margin (OD04410) | 4.1 | 0.0 |
| Prostate Cancer (OD04720-01) | 4.0 | 0.0 |
| Prostate Margin (OD04720-02) | 0.0 | 1.1 |
| Normal Lung 061010 | 0.0 | 3.9 |
| Lung Met to Muscle (ODO4286) | 0.0 | 2.6 |
| Muscle Margin (ODO4286) | 8.7 | 0.0 |
| Lung Malignant Cancer (OD03126) | 0.0 | 0.0 |
| Lung Margin (OD03126) | 0.0 | 0.5 |
| Lung Cancer (OD04404) | 8.0 | 21.9 |
| Lung Margin (OD04404) | 0.0 | 0.0 |
| Lung Cancer (OD04565) | 3.2 | 1.0 |
| Lung Margin (OD04565) | 1.8 | 1.2 |
| Lung Cancer (OD04237-01) | 9.0 | 4.4 |
| Lung Margin (OD04237-02) | 0.0 | 0.0 |
| Ocular Mel Met to Liver (ODO4310) | 0.0 | 0.0 |
| Liver Margin (ODO4310) | 0.0 | 0.0 |
| Melanoma Mets to Lung (OD04321) | 10.7 | 9.5 |
| Lung Margin (OD04321) | 0.0 | 0.0 |
| Normal Kidney | 10.4 | 4.8 |
| Kidney Ca, Nuclear grade 2 (OD04338) | 0.0 | 0.0 |
| Kidney Margin (OD04338) | 0.0 | 0.0 |
| Kidney Ca Nuclear grade 1/2 (OD04339) | 0.0 | 0.0 |
| Kidney Margin (OD04339) | 0.0 | 0.0 |
| Kidney Ca, Clear cell type (OD04340) | 0.0 | 0.0 |
| Kidney Margin (OD04340) | 0.0 | 0.0 |
| Kidney Ca, Nuclear grade 3 (OD04348) | 0.0 | 1.1 |
| Kidney Margin (OD04348) | 3.4 | 0.6 |
| Kidney Cancer (OD04622-01) | 0.0 | 1.1 |
| Kidney Margin (OD04622-03) | 0.0 | 0.0 |
| Kidney Cancer (OD04450-01) | 0.0 | 0.0 |
| Kidney Margin (OD04450-03) | 0.0 | 6.7 |
| Kidney Cancer 8120607 | 0.0 | 0.0 |
| Kidney Margin 8120608 | 0.0 | 0.0 |
| Kidney Cancer 8120613 | 0.0 | 0.0 |
| Kidney Margin 8120614 | 1.2 | 0.5 |
| Kidney Cancer 9010320 | 0.5 | 1.1 |
| Kidney Margin 9010321 | 0.0 | 0.0 |
| Normal Uterus | 0.0 | 0.6 |
| Uterus Cancer 064011 | 0.0 | 0.0 |
| Normal Thyroid | 1.8 | 0.0 |
| Thyroid Cancer 064010 | 0.0 | 0.0 |
| Thyroid Cancer A302152 | 5.0 | 0.0 |
| Thyroid Margin A302153 | 0.0 | 0.0 |
| Normal Breast | 10.5 | 5.3 |
| Breast Cancer (OD04566) | 2.1 | 4.3 |
| Breast Cancer (OD04590-01) | 1.3 | 1.0 |
| Breast Cancer Mets (OD04590-03) | 0.0 | 0.0 |
| Breast Cancer Metastasis (OD04655-05) | 0.0 | 0.0 |
| Breast Cancer 064006 | 17.3 | 8.7 |
| Breast Cancer 1024 | 10.1 | 11.7 |
| Breast Cancer 9100266 | 0.0 | 1.0 |
| Breast Margin 9100265 | 11.0 | 1.9 |
| Breast Cancer A209073 | 9.1 | 5.2 |
| Breast Margin A209073 | 17.0 | 19.8 |
| Normal Liver | 0.0 | 0.0 |
| Liver Cancer 064003 | 1.8 | 1.1 |
| Liver Cancer 1025 | 1.9 | 1.1 |
| Liver Cancer 1026 | 0.0 | 0.0 |
| Liver Cancer 6004-T | 0.0 | 0.0 |
| Liver Tissue 6004-N | 3.6 | 1.7 |
| Liver Cancer 6005-T | 0.0 | 0.0 |
| Liver Tissue 6005-N | 0.0 | 0.0 |
| Normal Bladder | 4.2 | 2.4 |
| Bladder Cancer 1023 | 0.0 | 4.3 |
| Bladder Cancer A302173 | 100.0 | 100.0 |
| Bladder Cancer (OD04718-01) | 1.7 | 12.4 |
| Bladder Normal Adjacent (OD04718-03) | 1.9 | 0.9 |
| Normal Ovary | 0.0 | 0.0 |
| Ovarian Cancer 064008 | 0.0 | 9.4 |
| Ovarian Cancer (OD04768-07) | 0.0 | 0.0 |
| Ovary Margin (OD04768-08) | 0.0 | 0.0 |
| Normal Stomach | 0.0 | 0.9 |
| Gastric Cancer 9060358 | 0.0 | 0.8 |
| Stomach Margin 9060359 | 0.0 | 0.0 |
| Gastric Cancer 9060395 | 1.3 | 0.0 |
| Stomach Margin 9060394 | 0.0 | 0.0 |
| Gastric Cancer 9060397 | 0.0 | 0.0 |
| Stomach Margin 9060396 | 0.0 | 0.0 |
| Gastric Cancer 064005 | 0.0 | 2.6 |

Column A - Rel. Exp. (%) Ag1528, Run 145158111
Column B - Rel. Exp. (%) Ag1528, Run 147327848

TABLE AAF
Panel 3D

| Tissue Name | A |
|---|---|
| Daoy- Medulloblastoma | 2.6 |
| TE671- Medulloblastoma | 0.0 |
| D283 Med- Medulloblastoma | 0.0 |
| PFSK-1- Primitive Neuroectodermal | 10.6 |
| XF-498- CNS | 81.8 |
| SNB-78- Glioma | 8.8 |
| SF-268- Glioblastoma | 11.9 |
| T98G- Glioblastoma | 1.8 |
| SK-N-SH- Neuroblastoma (metastasis) | 27.7 |
| SF-295- Glioblastoma | 0.0 |
| Cerebellum | 84.7 |
| Cerebellum | 7.1 |
| NCI-H292- Mucoepidermoid lung carcinoma | 1.0 |
| DMS-114- Small cell lung cancer | 0.0 |
| DMS-79- Small cell lung cancer | 38.4 |
| NCI-H146- Small cell lung cancer | 0.0 |
| NCI-H526- Small cell lung cancer | 24.5 |
| NCI-N417- Small cell lung cancer | 9.4 |
| NCI-H82- Small cell lung cancer | 20.0 |
| NCI-H157- Squamous cell lung cancer | 39.2 |
| (metastasis) | |
| NCI-H1155- Large cell lung cancer | 0.0 |
| NCI-H1299- Large cell lung cancer | 60.7 |
| NCI-H727- Lung carcinoid | 0.0 |
| NCI-UMC-11- Lung carcinoid | 0.0 |
| LX-1- Small cell lung cancer | 0.0 |
| Colo-205- Colon cancer | 0.0 |
| KM12- Colon cancer | 5.8 |
| KM20L2- Colon cancer | 0.0 |
| NCI-H716- Colon cancer | 0.0 |
| SW-48- Colon adenocarcinoma | 0.0 |
| SW1116- Colon adenocarcinoma | 0.0 |
| LS 174T- Colon adenocarcinoma | 0.0 |
| SW-948- Colon adenocarcinoma | 2.2 |
| SW-480- Colon adenocarcinoma | 0.0 |
| NCI-SNU-5- Gastric carcinoma | 2.0 |
| KATO III- Gastric carcinoma | 68.8 |
| NCI-SNU-16- Gastric carcinoma | 0.0 |
| NCI-SNU-1- Gastric carcinoma | 13.8 |
| RF-1- Gastric adenocarcinoma | 0.0 |
| RF-48- Gastric adenocarcinoma | 0.0 |
| MKN-45- Gastric carcinoma | 0.0 |
| NCI-N87- Gastric carcinoma | 0.0 |
| OVCAR-5- Ovarian carcinoma | 0.0 |
| RL95-2- Uterine carcinoma | 0.0 |
| HelaS3- Cervical adenocarcinoma | 0.0 |
| Ca Ski- Cervical epidermoid carcinoma | 40.6 |
| (metastasis) | |
| ES-2- Ovarian clear cell carcinoma | 29.7 |
| Ramos- Stimulated with PMA/ionomycin 6 h | 4.5 |
| Ramos- Stimulated with PMA/ionomycin 14 h | 9.4 |
| MEG-01- Chronic myelogenous leukemia | 1.9 |
| (megokaryoblast) | |
| Raji- Burkitt's lymphoma | 9.8 |
| Daudi- Burkitt's lymphoma | 100.0 |
| U266- B-cell plasmacytoma | 0.0 |
| CA46- Burkitt's lymphoma | 2.0 |
| RL- non-Hodgkin's B-cell lymphoma | 0.0 |
| JM1- pre-B-cell lymphoma | 0.0 |
| Jurkat- T cell leukemia | 0.0 |
| TF-1- Erythroleukemia | 1.6 |
| HUT 78- T-cell lymphoma | 0.0 |
| U937- Histiocytic lymphoma | 0.0 |
| KU-812- Myelogenous leukemia | 0.0 |
| 769-P- Clear cell renal carcinoma | 0.0 |
| Caki-2- Clear cell renal carcinoma | 0.0 |
| SW 839- Clear cell renal carcinoma | 0.0 |
| Rhabdoid kidney tumor | 0.0 |
| Hs766T- Pancreatic carcinoma (LN | 30.8 |
| metastasis) | |
| CAPAN-1- Pancreatic adenocarcinoma (liver | 6.8 |
| metastasis) | |
| SU86.86- Pancreatic carcinoma (liver | 50.7 |
| metastasis) | |
| BxPC-3- Pancreatic adenocarcinoma | 26.8 |
| HPAC- Pancreatic adenocarcinoma | 4.4 |
| MIA PaCa-2- Pancreatic carcinoma | 10.2 |
| CFPAC-1- Pancreatic ductal adenocarcinoma | 3.8 |
| PANC-1- Pancreatic epithelioid ductal | 12.7 |
| carcinoma | |
| T24- Bladder carcinma (transitional cell) | 33.4 |
| 5637- Bladder carcinoma | 24.1 |
| HT-1197- Bladder carcinoma | 19.9 |
| UM-UC-3- Bladder carcinma (transitional cell) | 5.1 |
| A204- Rhabdomyosarcoma | 0.0 |
| HT-1080- Fibrosarcoma | 95.3 |
| MG-63- Osteosarcoma | 0.0 |
| SK-LMS-1- Leiomyosarcoma (vulva) | 35.8 |
| SJRH30- Rhabdomyosarcoma (met to bone | 13.6 |
| marrow) | |
| A431- Epidermoid carcinoma | 0.0 |
| WM266-4- Melanoma | 12.3 |
| DU 145- Prostate carcinoma (brain | 0.0 |
| metastasis) | |
| MDA-MB-468- Breast adenocarcinoma | 5.1 |
| SCC-4- Squamous cell carcinoma of tongue | 0.0 |
| SCC-9- Squamous cell carcinoma of tongue | 0.0 |
| SCC-15- Squamous cell carcinoma of tongue | 0.0 |
| CAL 27- Squamous cell carcinoma of tongue | 0.0 |

Column A - Rel. Exp. (%) Ag1212, Run 164038901

TABLE AAG
Panel 4D

| Tissue Name | A | B | C | D |
|---|---|---|---|---|
| Secondary Th1 act | 0.1 | 0.0 | 0.0 | 0.0 |
| Secondary Th2 act | 0.0 | 0.0 | 0.0 | 0.0 |
| Secondary Tr1 act | 0.0 | 0.1 | 0.0 | 0.0 |
| Secondary Th1 rest | 0.0 | 0.0 | 0.0 | 0.0 |
| Secondary Th2 rest | 0.0 | 0.0 | 0.0 | 0.0 |
| Secondary Tr1 rest | 0.0 | 0.0 | 0.0 | 0.1 |
| Primary Th1 act | 0.0 | 0.0 | 0.0 | 0.0 |
| Primary Th2 act | 0.0 | 0.0 | 0.0 | 0.0 |
| Primary Tr1 act | 0.0 | 0.0 | 0.0 | 0.0 |
| Primary Th1 rest | 0.0 | 0.0 | 0.0 | 0.0 |
| Primary Th2 rest | 0.0 | 0.0 | 0.0 | 0.0 |
| Primary Tr1 rest | 0.0 | 0.0 | 0.0 | 0.0 |
| CD45RA CD4 lymphocyte act | 0.7 | 0.6 | 0.8 | 0.8 |
| CD45RO CD4 lymphocyte act | 0.0 | 0.0 | 0.0 | 0.0 |
| CD8 lymphocyte act | 0.0 | 0.0 | 0.0 | 0.0 |
| Secondary CD8 lymphocyte rest | 0.0 | 0.0 | 0.0 | 0.0 |
| Secondary CD8 lymphocyte act | 0.0 | 0.0 | 0.0 | 0.9 |
| CD4 lymphocyte none | 0.0 | 0.0 | 0.0 | 0.0 |
| 2ry Th1/Th2/Tr1_anti-CD95 CH11 | 0.0 | 0.0 | 0.0 | 0.0 |
| LAK cells rest | 0.0 | 0.0 | 0.0 | 0.0 |
| LAK cells IL-2 | 0.0 | 0.0 | 0.1 | 0.0 |
| LAK cells IL-2 + IL-12 | 0.2 | 0.0 | 0.0 | 0.1 |
| LAK cells IL-2 + IFN gamma | 0.0 | 0.2 | 0.0 | 0.0 |
| LAK cells IL-2 + IL-18 | 0.0 | 0.0 | 0.0 | 0.0 |
| LAK cells PMA/ionomycin | 0.0 | 0.0 | 0.0 | 0.0 |
| NK Cells IL-2 rest | 0.0 | 0.0 | 0.0 | 0.0 |
| Two Way MLR 3 day | 0.0 | 0.0 | 0.0 | 0.0 |
| Two Way MLR 5 day | 0.0 | 0.0 | 0.0 | 0.0 |
| Two Way MLR 7 day | 0.0 | 0.0 | 0.0 | 0.0 |
| PBMC rest | 0.0 | 0.0 | 0.0 | 0.0 |
| PBMC PWM | 0.0 | 1.1 | 0.2 | 0.0 |
| PBMC PHA-L | 0.0 | 0.0 | 0.0 | 0.1 |
| Ramos (B cell) none | 42.6 | 21.0 | 19.2 | 21.6 |
| Ramos (B cell) ionomycin | 100.0 | 100.0 | 100.0 | 100.0 |
| B lymphocytes PWM | 0.3 | 0.5 | 0.5 | 0.2 |
| B lymphocytes CD40L and IL-4 | 3.1 | 4.0 | 4.8 | 7.2 |
| EOL-1 dbcAMP | 0.0 | 0.0 | 0.0 | 0.0 |
| EOL-1 dbcAMP PMA/ionomycin | 0.0 | 0.0 | 0.0 | 0.0 |
| Dendritic cells none | 0.2 | 0.1 | 0.1 | 0.2 |
| Dendritic cells LPS | 0.0 | 0.0 | 0.0 | 0.0 |
| Dendritic cells anti-CD40 | 0.1 | 0.0 | 0.0 | 0.0 |
| Monocytes rest | 0.1 | 0.0 | 0.0 | 0.0 |
| Monocytes LPS | 0.1 | 0.0 | 0.0 | 0.0 |
| Macrophages rest | 0.9 | 0.0 | 0.0 | 0.0 |
| Macrophages LPS | 0.1 | 0.0 | 0.0 | 6.0 |
| HUVEC none | 3.8 | 4.0 | 3.8 | 2.9 |
| HUVEC starved | 6.7 | 5.1 | 6.3 | 5.0 |
| HUVEC IL-1beta | 2.3 | 1.3 | 2.8 | 1.0 |
| HUVEC IFN gamma | 2.2 | 2.0 | 2.2 | 1.3 |
| HUVEC TNF alpha + IFN gamma | 0.3 | 0.6 | 0.9 | 0.8 |
| HUVEC TNF alpha + IL4 | 1.0 | 1.3 | 2.3 | 1.4 |
| HUVEC IL-11 | 2.1 | 1.9 | 3.0 | 2.6 |
| Lung Microvascular EC none | 6.8 | 5.1 | 4.0 | 3.9 |
| Lung Microvascular EC TNFalpha + | 1.9 | 2.0 | 3.6 | 9.8 |
| IL-1beta | | | | |
| Microvascular Dermal EC none | 8.4 | 6.0 | 4.6 | 7.2 |
| Microsvasular Dermal EC TNFalpha + | 1.6 | 3.5 | 2.5 | 3.6 |
| IL-1beta | | | | |
| Bronchial epithelium TNFalpha + | 1.6 | 0.0 | 0.5 | 0.0 |
| IL1beta | | | | |
| Small airway epithelium none | 0.2 | 0.5 | 0.2 | 0.3 |
| Small airway epithelium TNFalpha + | 2.8 | 1.9 | 1.8 | 2.2 |
| IL-1beta | | | | |
| Coronery artery SMC rest | 1.7 | 1.6 | 1.2 | 0.5 |
| Coronery artery SMC TNFalpha + | 0.6 | 0.9 | 1.0 | 0.2 |
| IL-1beta | | | | |
| Astrocytes rest | 0.9 | 2.0 | 0.6 | 0.8 |
| Astrocytes TNFalpha + IL-1beta | 0.7 | 0.2 | 0.5 | 0.7 |
| KU-812 (Basophil) rest | 0.1 | 0.0 | 0.0 | 0.0 |
| KU-812 (Basophil) PMA/ionomycin | 0.0 | 0.0 | 0.0 | 0.0 |
| CCD1106 (Keratinocytes) none | 3.0 | 4.5 | 4.1 | 3.5 |
| CCD1106 (Keratinocytes) TNFalpha + | 11.7 | 0.7 | 1.0 | 0.4 |
| IL-1beta | | | | |
| Liver cirrhosis | 1.5 | 1.0 | 0.7 | 0.1 |
| Lupus kidney | 0.0 | 0.0 | 0.0 | 0.0 |
| NCI-H292 none | 0.0 | 0.1 | 0.0 | 0.0 |
| NCI-H292 IL-4 | 0.5 | 2.3 | 0.3 | 0.2 |
| NCI-H292 IL-9 | 0.1 | 0.1 | 0.2 | 0.3 |
| NCI-H292 IL-13 | 0.2 | 0.7 | 0.2 | 0.0 |
| NCI-H292 IFN gamma | 0.0 | 0.1 | 0.0 | 0.1 |
| HPAEC none | 7.6 | 8.7 | 8.2 | 7.5 |
| HPAEC TNF alpha + IL-1 beta | 2.0 | 2.7 | 3.0 | 2.0 |
| Lung fibroblast none | 0.3 | 0.2 | 0.0 | 0.2 |
| Lung fibroblast TNF alpha + | 0.0 | 0.1 | 0.0 | 0.0 |
| IL-1 beta | | | | |
| Lung fibroblast IL-4 | 0.1 | 0.3 | 0.5 | 0.1 |
| Lung fibroblast IL-9 | 0.3 | 1.0 | 0.6 | 0.1 |
| Lung fibroblast IL-13 | 1.4 | 0.7 | 0.1 | 0.3 |
| Lung fibroblast IFN gamma | 0.0 | 0.4 | 0.2 | 0.3 |
| Dermal fibroblast CCD1070 rest | 11.4 | 8.8 | 8.7 | 4.9 |
| Dermal fibroblast CCD1070 TNF alpha | 6.7 | 8.1 | 9.2 | 8.5 |
| Dermal fibroblast CCD1070 IL-1 beta | 4.5 | 2.1 | 2.5 | 4.7 |
| Dermal fibroblast IFN gamma | 0.8 | 1.4 | 1.0 | 0.1 |
| Dermal fibroblast IL-4 | 1.0 | 1.1 | 1.3 | 1.4 |
| IBD Colitis 2 | 0.0 | 0.1 | 0.1 | 0.2 |
| IBD Crohn's | 0.2 | 0.0 | 0.0 | 0.0 |
| Colon | 0.0 | 0.0 | 0.1 | 0.0 |
| Lung | 0.0 | 0.1 | 0.0 | 0.0 |
| Thymus | 0.5 | 0.3 | 0.2 | 0.4 |
| Kidney | 0.1 | 0.1 | 0.0 | 0.0 |

Column A - Rel. Exp. (%) Ag1212, Run 140407622
Column B - Rel. Exp. (%) Ag1528, Run 147327849
Column C - Rel. Exp. (%) Ag1528, Run 148106604
Column D - Rel. Exp. (%) Ag1528, Run 162699795

CNS_neurodegeneration_v1.0 Summary: Ag1212 Highest expression of this gene, a homolog of calgizzarin, was seen in the hippocampus of a patient with Alzheimer's disease. The gene was also expressed in numerous regions associated with Alzheimer's neurodegeneration including the hippocampus, cortex and amygdala, as seen below in Panels 1.2 and 1.3D. Calgizzarin (S100C, S100A11) accumulation in the nucleus is associated with cellular senescence, which may be critical in aging and age-related disorders such as Alzheimer's disease. Agents that bind S100C are cardioprotective and have been proven useful in extending life. Agents that block the action or nuclear localization of this gene or gene product are useful in the treatment of Alzheimer's disease and are effective in prolonging lifespan.

Panel 1.3D Summary: Ag1212/Ag1528 Highest expression of this gene was detected in the cortex, the fetal brain, and the hippocampus (CTs=31-33), with moderate expression across many regions of the brain. In addition, a substantial number of samples derived from melanoma and brain cancer cell lines showed expression of this gene. Thus, the expression of this gene is useful as a marker to distinguish melanoma cell lines, brain cancer cell lines and brain tissue from other samples in the panel. Therapeutic modulation of this gene or gene product, through the use of small molecule drugs, antibodies or protein therapeutics, is of benefit in the treatment of melanoma or brain cancer.

Panel 2D Summary: Ag1528 Highest expression of this gene was observed in bladder cancer (CTs=31), with significant over-expression in the bladder cancer samples when compared to expression in the normal adjacent tissue. There was also substantial expression in a cluster of samples derived from breast tissue. Expression of this gene is useful as a marker to distinguish bladder cancer and breast tissue from other samples in the panel. Therapeutic modulation of the expression of this gene or this gene product, through the use of small molecule drugs, antibodies or protein therapeutics is of use in the treatment of bladder cancer or breast disease.

Panel 3D Summary: Ag1212 Highest expression of this gene was seen in a cell line derived from a lymphoma cell line (Daudi) (CT=31.2). Low but significant expression was also seen in cell lines derived from brain cancer and lung cancer, as is seen in Panel 1.3D.

Panel 4D Summary: Ag1212/Ag1528 Highest expression of this gene was seen in ionomycin treated Ramos B cells (CTs=26-28). Lower levels of expression were also seen in dermal fibroblasts, HUVEC endothelial cells, and keratinocytes. The expression in keratinocytes of this gene product, a homolog of calgizzarin, is consistent with Robinson (J. Biol. Chem. 272: 12035.) Therapeutic modulation of the expression or function of this gene or gene product, through the use antibodies or protein therapeutics, is beneficial in the treatment of disease states associated with hyperactivation of keratinocytes. Higher expression of the gene product in a B cell line compared to the expression in a primary B cell line shows a role for the protein encoded by this gene in cell growth and/or differentiation. Therapeutic modulation of the expression or function of this gene product, through the use antibodies is beneficial in the treatment of B cell lymphomas.

AB. CG55033-01 and CG55033-04 and CG55033-05: LRR Domain Containing Protein

Expression of gene CG55033-01 and variants CG55033-04 and CG55033-05 was assessed using the primer-probe sets Ag1094, Ag6719 and Ag6725, described in Tables ABA, ABB and ABC. Results of the RTQ-PCR runs are shown in Tables ABE, ABF, ABG and ABH. Please note that Ag6725 is specific for the CG55033-04 and CG55033-05 variants only.

TABLE ABA
Probe Name Ag1094

| | | | | SEQ |
|---|---|---|---|---|
| | | | Start | ID |
| Primers | Sequences | Length | Position | No |
| Forward | 5′-atggactggaaaacctggaa-3′ | 20 | 398 | 1393 |
| Probe | TET-5′-tcctgcaagcagataacaattttatcaca-3′- | 29 | 419 | 1394 |
| | TAMRA | | | |
| Reverse | 5′-tgctaaaggcacttggttca-3′ | 20 | 453 | 1395 |

TABLE ABB
Probe Name Ag6719

| | | | | SEQ |
|---|---|---|---|---|
| | | | Start | ID |
| Primers | Sequences | Length | Position | No |
| Forward | 5′-ggaaaatcattcaccactcaca-3′ | 22 | 1575 | 1396 |
| Probe | TET-5′-acaaaaccacgaaccaatcaacagaa-3′- | 26 | 1613 | 1397 |
| | TAMRA | | | |
| Reverse | 5′-tctgtacaatgagctggcatct-3′ | 22 | 1653 | 1398 |

TABLE ABC
Probe Name Ag6725

| | | | | |
|---|---|---|---|---|
| | | | | SEQ |
| | | | Start | ID |
| Primers | Sequences | Length | Position | No |

| | | | | |
|---|---|---|---|---|
| Forward | 5′-gaagctgtggattcatctctttt-3′ | 23 | 3 | 1399 |
| Probe | TET-5′-catctctccttgcctgtatatctttacactcc- | 32 | 29 | 1400 |
| | TAMRA | | | |
| Reverse | 5′-ggatgagagcactggagtttg-3′ | 21 | 61 | 1401 |

TABLE ABD
Ardais Panel v.1.0

| Tissue Name | A |
|---|---|
| 136799_Lung cancer(362) | 0.1 |
| 136800_Lung NAT(363) | 4.8 |
| 136813_Lung cancer(372) | 37.4 |
| 136814_Lung NAT(373) | 2.8 |
| 136815_Lung cancer(374) | 19.9 |
| 136816_Lung NAT(375) | 4.3 |
| 136791_Lung cancer(35A) | 0.5 |
| 136795_Lung cancer(35E) | 100.0 |
| 136797_Lung cancer(360) | 0.2 |
| 136794_Lung NAT(35D) | 5.0 |
| 136818_Lung NAT(377) | 5.4 |
| 136787_lung cancer(356) | 0.2 |
| 136788_Lung NAT(357) | 0.7 |
| 136804_Lung cancer(369) | 1.1 |
| 136805_Lung NAT(36A) | 2.5 |
| 136806_Lung cancer(36B) | 18.2 |
| 136807_Lung NAT(36C) | 2.4 |
| 136789_lung cancer(358) | 0.7 |
| 136802_Lung cancer(365) | 0.1 |
| 136803_Lung cancer(368) | 1.1 |
| 136811_Lung cancer(370) | 4.5 |
| 136810_Lung NAT(36F) | 32.1 |

Column A - Rel. Exp. (%) Ag1094, Run 263147830

TABLE ABE
General_screening_panel_v1.6

| Tissue Name | A |
|---|---|
| Adipose | 0.7 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.6 |
| Squamous cell carcinoma SCC-4 | 7.4 |
| Testis Pool | 0.5 |
| Prostate ca.* (bone met) PC-3 | 28.1 |
| Prostate Pool | 6.2 |
| Placenta | 0.0 |
| Uterus Pool | 0.3 |
| Ovarian ca. OVCAR-3 | 0.4 |
| Ovarian ca. SK-OV-3 | 0.4 |
| Ovarian ca. OVCAR-4 | 0.0 |
| Ovarian ca. OVCAR-5 | 10.3 |
| Ovarian ca. IGROV-1 | 10.2 |
| Ovarian ca. OVCAR-8 | 1.0 |
| Ovary | 0.0 |
| Breast ca. MCF-7 | 0.8 |
| Breast ca. MDA-MB-231 | 0.0 |
| Breast ca. BT 549 | 0.9 |
| Breast ca. T47D | 0.0 |
| Breast ca. MDA-N | 0.0 |
| Breast Pool | 1.2 |
| Trachea | 4.6 |
| Lung | 0.0 |
| Fetal Lung | 14.4 |
| Lung ca. NCI-N417 | 0.0 |
| Lung ca. LX-1 | 0.0 |
| Lung ca. NCI-H146 | 16.8 |
| Lung ca. SHP-77 | 100.0 |
| Lung ca. A549 | 2.7 |
| Lung ca. NCI-H526 | 0.0 |
| Lung ca. NCI-H23 | 12.9 |
| Lung ca. NCI-H460 | 0.0 |
| Lung ca. HOP-62 | 3.2 |
| Lung ca. NCI-H522 | 0.1 |
| Liver | 0.0 |
| Fetal Liver | 2.7 |
| Liver ca. HepG2 | 0.0 |
| Kidney Pool | 0.4 |
| Fetal Kidney | 7.8 |
| Renal ca. 786-0 | 0.0 |
| Renal ca. A498 | 0.5 |
| Renal ca. ACHN | 0.0 |
| Renal ca. UO-31 | 0.0 |
| Renal ca. TK-10 | 0.0 |
| Bladder | 0.1 |
| Gastric ca. (liver met.) NCI-N87 | 9.5 |
| Gastric ca. KATO III | 9.5 |
| Colon ca. SW-948 | 1.0 |
| Colon ca. SW480 | 0.0 |
| Colon ca.* (SW480 met) SW620 | 0.4 |
| Colon ca. HT29 | 15.5 |
| Colon ca. HCT-116 | 0.0 |
| Colon ca. CaCo-2 | 0.0 |
| Colon cancer tissue | 0.0 |
| Colon ca. SW1116 | 0.0 |
| Colon ca. Colo-205 | 0.0 |
| Colon ca. SW-48 | 0.0 |
| Colon Pool | 1.0 |
| Small Intestine Pool | 0.9 |
| Stomach Pool | 1.1 |
| Bone Marrow Pool | 0.6 |
| Fetal Heart | 0.9 |
| Heart Pool | 0.2 |
| Lymph Node Pool | 0.5 |
| Fetal Skeletal Muscle | 0.6 |
| Skeletal Muscle Pool | 0.0 |
| Spleen Pool | 0.0 |
| Thymus Pool | 1.5 |
| CNS cancer (glio/astro) U87-MG | 0.2 |
| CNS cancer (glio/astro) U-118-MG | 0.7 |
| CNS cancer (neuro; met) SK-N-AS | 18.0 |
| CNS cancer (astro) SF-539 | 0.0 |
| CNS cancer (astro) SNB-75 | 6.3 |
| CNS cancer (glio) SNB-19 | 7.4 |
| CNS cancer (glio) SF-295 | 68.3 |
| Brain (Amygdala) Pool | 0.6 |
| Brain (cerebellum) | 0.2 |
| Brain (fetal) | 0.7 |
| Brain (Hippocampus) Pool | 0.3 |
| Cerebral Cortex Pool | 0.1 |
| Brain (Substantia nigra) Pool | 0.1 |
| Brain (Thalamus) Pool | 0.8 |
| Brain (whole) | 0.1 |
| Spinal Cord Pool | 0.3 |
| Adrenal Gland | 0.0 |
| Pituitary gland Pool | 0.0 |
| Salivary Gland | 0.5 |
| Thyroid (female) | 0.0 |
| Pancreatic ca. CAPAN2 | 0.0 |
| Pancreas Pool | 0.0 |

Column A - Rel. Exp. (%) Ag6719, Run 277223815

TABLE ABF
Panel 2D

| Tissue Name | A | B |
|---|---|---|
| Normal Colon | 12.4 | 11.4 |
| CC Well to Mod Diff (ODO3866) | 0.1 | 0.0 |
| CC Margin (ODO3866) | 1.0 | 1.5 |
| CC Gr.2 rectosigmoid (ODO3868) | 0.3 | 0.1 |
| CC Margin (ODO3868) | 0.4 | 0.2 |
| CC Mod Diff (ODO3920) | 0.1 | 0.1 |
| CC Margin (ODO3920) | 0.8 | 0.8 |
| CC Gr.2 ascend colon (ODO3921) | 2.4 | 2.2 |
| CC Margin (ODO3921) | 2.0 | 1.9 |
| CC from Partial Hepatectomy | 0.0 | 0.0 |
| (ODO4309) Mets | | |
| Liver Margin (ODO4309) | 0.2 | 0.3 |
| Colon mets to lung (OD04451-01) | 0.1 | 0.1 |
| Lung Margin (OD04451-02) | 0.9 | 0.6 |
| Normal Prostate 6546-1 | 2.7 | 3.0 |
| Prostate Cancer (OD04410) | 1.5 | 1.4 |
| Prostate Margin (OD04410) | 6.5 | 8.0 |
| Prostate Cancer (OD04720-01) | 5.9 | 6.1 |
| Prostate Margin (OD04720-02) | 14.1 | 12.6 |
| Normal Lung 061010 | 3.3 | 3.5 |
| Lung Met to Muscle (ODO4286) | 0.2 | 0.2 |
| Muscle Margin (ODO4286) | 0.0 | 0.0 |
| Lung Malignant Cancer (OD03126) | 7.9 | 6.0 |
| Lung Margin (OD03126) | 1.8 | 2.5 |
| Lung Cancer (OD04404) | 24.8 | 21.6 |
| Lung Margin (OD04404) | 1.8 | 1.7 |
| Lung Cancer (OD04565) | 0.7 | 1.2 |
| Lung Margin (OD04565) | 0.5 | 0.7 |
| Lung Cancer (OD04237-01) | 13.5 | 12.5 |
| Lung Margin (OD04237-02) | 1.4 | 1.0 |
| Ocular Mel Met to Liver (ODO4310) | 0.0 | 0.0 |
| Liver Margin (ODO4310) | 0.2 | 0.4 |
| Melanoma Mets to Lung (OD04321) | 0.4 | 0.2 |
| Lung Margin (OD04321) | 2.5 | 1.2 |
| Normal Kidney | 0.2 | 0.1 |
| Kidney Ca, Nuclear grade 2 (OD04338) | 0.0 | 0.0 |
| Kidney Margin (OD04338) | 0.2 | 0.2 |
| Kidney Ca Nuclear grade 1/2 (OD04339) | 0.0 | 0.0 |
| Kidney Margin (OD04339) | 0.1 | 0.0 |
| Kidney Ca, Clear cell type (OD04340) | 0.1 | 0.2 |
| Kidney Margin (OD04340) | 0.1 | 0.2 |
| Kidney Ca, Nuclear grade 3 (OD04348) | 0.0 | 0.0 |
| Kidney Margin (OD04348) | 0.0 | 0.1 |
| Kidney Cancer (OD04622-01) | 0.4 | 0.4 |
| Kidney Margin (OD04622-03) | 0.0 | 0.0 |
| Kidney Cancer (OD04450-01) | 0.0 | 0.0 |
| Kidney Margin (OD04450-03) | 0.0 | 0.1 |
| Kidney Cancer 8120607 | 0.4 | 0.3 |
| Kidney Margin 8120608 | 0.0 | 0.0 |
| Kidney Cancer 8120613 | 0.0 | 0.0 |
| Kidney Margin 8120614 | 0.0 | 0.0 |
| Kidney Cancer 9010320 | 0.1 | 0.0 |
| Kidney Margin 9010321 | 0.0 | 0.0 |
| Normal Uterus | 0.1 | 0.0 |
| Uterus Cancer 064011 | 0.5 | 0.6 |
| Normal Thyroid | 0.5 | 0.4 |
| Thyroid Cancer 064010 | 0.0 | 0.1 |
| Thyroid Cancer A302152 | 0.1 | 0.1 |
| Thyroid Margin A302153 | 0.2 | 0.1 |
| Normal Breast | 5.5 | 5.6 |
| Breast Cancer (OD04566) | 0.5 | 0.7 |
| Breast Cancer (OD04590-01) | 3.0 | 3.9 |
| Breast Cancer Mets (OD04590-03) | 1.4 | 1.7 |
| Breast Cancer Metastasis (OD04655-05) | 100.0 | 100.0 |
| Breast Cancer 064006 | 1.7 | 2.0 |
| Breast Cancer 1024 | 0.8 | 0.8 |
| Breast Cancer 9100266 | 3.5 | 4.1 |
| Breast Margin 9100265 | 3.5 | 4.2 |
| Breast Cancer A209073 | 0.7 | 0.7 |
| Breast Margin A209073 | 1.1 | 1.3 |
| Normal Liver | 2.0 | 1.7 |
| Liver Cancer 064003 | 0.0 | 0.1 |
| Liver Cancer 1025 | 0.3 | 0.3 |
| Liver Cancer 1026 | 0.0 | 0.0 |
| Liver Cancer 6004-T | 0.3 | 0.1 |
| Liver Tissue 6004-N | 0.0 | 0.1 |
| Liver Cancer 6005-T | 0.0 | 0.0 |
| Liver Tissue 6005-N | 0.1 | 0.1 |
| Normal Bladder | 0.2 | 0.1 |
| Bladder Cancer 1023 | 3.0 | 3.1 |
| Bladder Cancer A302173 | 1.0 | 0.8 |
| Bladder Cancer (OD04718-01) | 0.0 | 0.1 |
| Bladder Normal Adjacent (OD04718-03) | 4.1 | 3.4 |
| Normal Ovary | 0.1 | 0.0 |
| Ovarian Cancer 064008 | 1.6 | 1.4 |
| Ovarian Cancer (OD04768-07) | 0.0 | 0.0 |
| Ovary Margin (OD04768-08) | 0.1 | 0.0 |
| Normal Stomach | 1.0 | 1.7 |
| Gastric Cancer 9060358 | 0.2 | 0.2 |
| Stomach Margin 9060359 | 0.1 | 0.2 |
| Gastric Cancer 9060395 | 0.4 | 0.7 |
| Stomach Margin 9060394 | 0.4 | 0.4 |
| Gastric Cancer 9060397 | 0.1 | 0.3 |
| Stomach Margin 9060396 | 0.1 | 0.2 |
| Gastric Cancer 064005 | 1.0 | 1.3 |

Column A - Rel. Exp. (%) Ag1094, Run 148015599
Column B - Rel. Exp. (%) Ag1094, Run 148572893

TABLE ABG
Panel 3D

| Tissue Name | A |
|---|---|
| Daoy- Medulloblastoma | 0.0 |
| TE671- Medulloblastoma | 0.2 |
| D283 Med- Medulloblastoma | 0.2 |
| PFSK-1- Primitive Neuroectodermal | 1.6 |
| XF-498- CNS | 30.4 |
| SNB-78- Glioma | 0.7 |
| SF-268- Glioblastoma | 0.0 |
| T98G- Glioblastoma | 3.3 |
| SK-N-SH- Neuroblastoma (metastasis) | 22.4 |
| SF-295- Glioblastoma | 27.2 |
| Cerebellum | 6.7 |
| Cerebellum | 0.0 |
| NCI-H292- Mucoepidermoid lung carcinoma | 21.9 |
| DMS-114- Small cell lung cancer | 2.4 |
| DMS-79- Small cell lung cancer | 0.0 |
| NCI-H146- Small cell lung cancer | 100.0 |
| NCI-H526- Small cell lung cancer | 0.0 |
| NCI-N417- Small cell lung cancer | 0.0 |
| NCI-H82- Small cell lung cancer | 0.3 |
| NCI-H157- Squamous cell lung cancer (metastasis) | 0.0 |
| NCI-H1155- Large cell lung cancer | 65.1 |
| NCI-H1299- Large cell lung cancer | 0.0 |
| NCI-H727- Lung carcinoid | 13.8 |
| NCI-UMC-11- Lung carcinoid | 28.7 |
| LX-1- Small cell lung cancer | 0.7 |
| Colo-205- Colon cancer | 0.0 |
| KM12- Colon cancer | 0.1 |
| KM20L2- Colon cancer | 7.3 |
| NCI-H716- Colon cancer | 80.1 |
| SW-48- Colon adenocarcinoma | 0.3 |
| SW1116- Colon adenocarcinoma | 0.0 |
| LS 174T- Colon adenocarcinoma | 0.0 |
| SW-948- Colon adenocarcinoma | 0.6 |
| SW-480- Colon adenocarcinoma | 0.0 |
| NCI-SNU-5- Gastric carcinoma | 0.0 |
| KATO III- Gastric carcinoma | 0.0 |
| NCI-SNU-16- Gastric carcinoma | 1.7 |
| NCI-SNU-1- Gastric carcinoma | 0.0 |
| RF-1- Gastric adenocarcinoma | 0.0 |
| RF-48- Gastric adenocarcinoma | 0.0 |
| MKN-45- Gastric carcinoma | 0.1 |
| NCI-N87- Gastric carcinoma | 2.4 |
| OVCAR-5- Ovarian carcinoma | 0.0 |
| RL95-2- Uterine carcinoma | 2.8 |
| HelaS3- Cervical adenocarcinoma | 0.2 |
| Ca Ski- Cervical epidermoid carcinoma | 0.1 |
| (metastasis) | |
| ES-2- Ovarian clear cell carcinoma | 0.0 |
| Ramos- Stimulated with PMA/ionomycin 6 h | 0.1 |
| Ramos- Stimulated with PMA/ionomycin 14 h | 0.0 |
| MEG-01- Chronic myelogenous leukemia | 1.7 |
| (megokaryoblast) | |
| Raji- Burkitt's lymphoma | 0.0 |
| Daudi- Burkitt's lymphoma | 0.0 |
| U266- B-cell plasmacytoma | 0.2 |
| CA46- Burkitt's lymphoma | 0.0 |
| RL- non-Hodgkin's B-cell lymphoma | 0.1 |
| JM1- pre-B-cell lymphoma | 0.0 |
| Jurkat- T cell leukemia | 0.0 |
| TF-1- Erythroleukemia | 2.1 |
| HUT 78- T-cell lymphoma | 0.0 |
| U937- Histiocytic lymphoma | 0.0 |
| KU-812- Myelogenous leukemia | 8.2 |
| 769-P- Clear cell renal carcinoma | 0.0 |
| Caki-2- Clear cell renal carcinoma | 0.3 |
| SW 839- Clear cell renal carcinoma | 0.0 |
| Rhabdoid kidney tumor | 0.0 |
| Hs766T- Pancreatic carcinoma (LN metastasis) | 4.9 |
| CAPAN-1- Pancreatic adenocarcinoma (liver | 0.3 |
| metastasis) | |
| SU86.86- Pancreatic carcinoma (liver metastasis) | 1.5 |
| BxPC-3- Pancreatic adenocarcinoma | 23.7 |
| HPAC- Pancreatic adenocarcinoma | 76.8 |
| MIA PaCa-2- Pancreatic carcinoma | 0.5 |
| CFPAC-1- Pancreatic ductal adenocarcinoma | 0.5 |
| PANC-1- Pancreatic epithelioid ductal carcinoma | 6.9 |
| T24- Bladder carcinma (transitional cell) | 4.1 |
| 5637- Bladder carcinoma | 1.0 |
| HT-1197- Bladder carcinoma | 2.4 |
| UM-UC-3- Bladder carcinma (transitional cell) | 0.0 |
| A204- Rhabdomyosarcoma | 0.3 |
| HT-1080- Fibrosarcoma | 0.2 |
| MG-63- Osteosarcoma | 0.2 |
| SK-LMS-1- Leiomyosarcoma (vulva) | 0.0 |
| SJRH30- Rhabdomyosarcoma (met to bone marrow) | 0.2 |
| A431- Epidermoid carcinoma | 0.0 |
| WM266-4- Melanoma | 0.4 |
| DU 145- Prostate carcinoma (brain metastasis) | 0.0 |
| MDA-MB-468- Breast adenocarcinoma | 1.3 |
| SCC-4- Squamous cell carcinoma of tongue | 0.2 |
| SCC-9- Squamous cell carcinoma of tongue | 0.0 |
| SCC-15- Squamous cell carcinoma of tongue | 0.3 |
| CAL 27- Squamous cell carcinoma of tongue | 1.6 |

Column A - Rel. Exp. (%) Ag1094, Run 165022642

TABLE ABH
Panel 4.1D

| Tissue Name | A | B |
|---|---|---|
| Secondary Th1 act | 0.0 | 0.0 |
| Secondary Th2 act | 0.0 | 0.0 |
| Secondary Tr1 act | 0.0 | 0.0 |
| Secondary Th1 rest | 0.0 | 0.0 |
| Secondary Th2 rest | 0.0 | 0.0 |
| Secondary Tr1 rest | 0.0 | 0.0 |
| Primary Th1 act | 0.0 | 0.0 |
| Primary Th2 act | 0.0 | 0.0 |
| Primary Tr1 act | 0.0 | 0.0 |
| Primary Th1 rest | 0.0 | 0.0 |
| Primary Th2 rest | 0.0 | 0.0 |
| Primary Tr1 rest | 0.0 | 0.0 |
| CD45RA CD4 lymphocyte act | 0.0 | 0.0 |
| CD45RO CD4 lymphocyte act | 0.0 | 0.0 |
| CD8 lymphocyte act | 0.0 | 0.0 |
| Secondary CD8 lymphocyte rest | 0.0 | 0.0 |
| Secondary CD8 lymphocyte act | 0.0 | 0.0 |
| CD4 lymphocyte none | 0.0 | 0.0 |
| 2ry Th1/Th2/Tr1_anti-CD95 CH11 | 0.0 | 0.0 |
| LAK cells rest | 0.0 | 0.0 |
| LAK cells IL-2 | 0.0 | 0.0 |
| LAK cells IL-2 + IL-12 | 0.0 | 0.0 |
| LAK cells IL-2 + IFN gamma | 0.0 | 0.0 |
| LAK cells IL-2 + IL-18 | 0.0 | 0.0 |
| LAK cells PMA/ionomycin | 0.0 | 0.0 |
| NK Cells IL-2 rest | 0.0 | 0.0 |
| Two Way MLR 3 day | 0.8 | 0.0 |
| Two Way MLR 5 day | 0.0 | 0.0 |
| Two Way MLR 7 day | 0.0 | 0.0 |
| PBMC rest | 0.0 | 0.0 |
| PBMC PWM | 0.0 | 0.0 |
| PBMC PHA-L | 0.0 | 0.0 |
| Ramos (B cell) none | 0.0 | 0.0 |
| Ramos (B cell) ionomycin | 0.6 | 0.5 |
| B lymphocytes PWM | 0.0 | 0.0 |
| B lymphocytes CD40L and IL-4 | 0.0 | 0.0 |
| EOL-1 dbcAMP | 0.0 | 0.0 |
| EOL-1 dbcAMP PMA/ionomycin | 0.0 | 0.0 |
| Dendritic cells none | 0.0 | 0.0 |
| Dendritic cells LPS | 0.0 | 0.0 |
| Dendritic cells anti-CD40 | 0.0 | 0.0 |
| Monocytes rest | 0.0 | 0.0 |
| Monocytes LPS | 0.0 | 0.0 |
| Macrophages rest | 0.0 | 0.0 |
| Macrophages LPS | 0.0 | 0.0 |
| HUVEC none | 0.0 | 0.0 |
| HUVEC starved | 0.0 | 0.0 |
| HUVEC IL-1beta | 0.0 | 0.0 |
| HUVEC IFN gamma | 0.0 | 0.0 |
| HUVEC TNF alpha + IFN gamma | 0.0 | 0.0 |
| HUVEC TNF alpha + IL4 | 0.0 | 0.0 |
| HUVEC IL-11 | 0.0 | 0.0 |
| Lung Microvascular EC none | 0.0 | 0.0 |
| Lung Microvascular EC TNFalpha + IL-1beta | 0.0 | 0.0 |
| Microvascular Dermal EC none | 0.0 | 0.0 |
| Microsvasular Dermal EC TNFalpha + IL-1beta | 0.0 | 0.0 |
| Bronchial epithelium TNFalpha + IL1beta | 2.3 | 4.0 |
| Small airway epithelium none | 3.7 | 3.2 |
| Small airway epithelium TNFalpha + IL-1beta | 2.5 | 1.2 |
| Coronery artery SMC rest | 0.0 | 0.0 |
| Coronery artery SMC TNFalpha + IL-1beta | 0.0 | 0.0 |
| Astrocytes rest | 1.9 | 2.5 |
| Astrocytes TNFalpha + IL-1beta | 0.0 | 0.0 |
| KU-812 (Basophil) rest | 11.3 | 1.7 |
| KU-812 (Basophil) PMA/ionomycin | 61.1 | 100.0 |
| CCD1106 (Keratinocytes) none | 100.0 | 71.7 |
| CCD1106 (Keratinocytes) TNFalpha + IL-1beta | 7.6 | 4.0 |
| Liver cirrhosis | 0.8 | 0.2 |
| NCI-H292 none | 7.2 | 6.4 |
| NCI-H292 IL-4 | 4.6 | 6.8 |
| NCI-H292 IL-9 | 25.0 | 8.4 |
| NCI-H292 IL-13 | 9.7 | 7.8 |
| NCI-H292 IFN gamma | 2.8 | 1.9 |
| HPAEC none | 0.0 | 0.0 |
| HPAEC TNF alpha + IL-1 beta | 0.0 | 0.0 |
| Lung fibroblast none | 85.3 | 53.6 |
| Lung fibroblast TNF alpha + IL-1 beta | 35.1 | 31.0 |
| Lung fibroblast IL-4 | 20.7 | 7.8 |
| Lung fibroblast IL-9 | 2.8 | 7.3 |
| Lung fibroblast IL-13 | 11.4 | 3.0 |
| Lung fibroblast IFN gamma | 52.1 | 38.2 |
| Dermal fibroblast CCD1070 rest | 0.3 | 0.0 |
| Dermal fibroblast CCD1070 TNF alpha | 0.0 | 1.0 |
| Dermal fibroblast CCD1070 IL-1 beta | 0.0 | 0.0 |
| Dermal fibroblast IFN gamma | 0.0 | 0.0 |
| Dermal fibroblast IL-4 | 0.0 | 0.0 |
| Dermal Fibroblasts rest | 0.0 | 0.0 |
| Neutrophils TNFa + LPS | 0.0 | 0.0 |
| Neutrophils rest | 0.0 | 0.0 |
| Colon | 1.5 | 0.5 |
| Lung | 1.1 | 0.7 |
| Thymus | 1.4 | 0.0 |
| Kidney | 1.6 | 0.0 |

Column A - Rel. Exp. (%) Ag6719, Run 276596890
Column B - Rel. Exp. (%) Ag6725, Run 276846995

Ardais Panel v.1.0 Summary: Ag1094 Highest expression of this gene was seen in a lung cancer sample (CT=23.5). High to moderate expression of this gene was seen in a number of normal and cancerous samples from lung. Expression of this gene was higher in four cancer samples compared to normal lung samples. Expression of this gene is useful as a marker to detect the presence of lung cancer and therapeutic modulation of this gene or its protein product is useful in the treatment of lung cancer.

General_screening₁₃ panel_v1.6 Summary: Ag6719 Highest expression of this gene was seen in a lung cancer SHP-77 cell line (CT=26.6). High expression of this gene was mainly seen in cancer cell lines derived from brain, lung, colon, gastric, ovarian and prostate cancers. Expression was higher in these cell lines compared to the normal tissues. Expression of this gene is useful as a marker to detect the presence of these cancers and also, therapeutic modulation of this gene or its protein product is useful in the treatment of brain, lung, colon, gastric, ovarian and prostate cancers.

Low expression of this gene was also seen in fetal lung, fetal kidney, and fetal heart. Expression was higher in these fetal tissues compared to expression in the corresponding adult tissues. This gene plays a role in early development of these tissue. Therapeutic modulation of this gene or its protein product is useful in the treatment of diseases related to development of these tissues.

This gene encodes a novel insulin-like growth factor binding protein acid labile subunit. Low expression of this gene was also seen in fetal brain and thalamus. Therefore, therapeutic modulation of this gene or gene product is useful in the treatment of neurological disorders related to thalamus and brain development. Insulin-like growth factor (IGF) has neuroprotective effects, and is currently under investigation as a biopharmaceutical for the treatment of amyotropic lateral sclerosis (Mewar R. J. Neurosci. Res 50:721-728, PMID: 9418960; Corse A. M., Neurobiol. Dis. 6:335-346). In serum, IGF is bound to both IGF-binding protein (IGFBP) and the acid labile subunit (IGFBP-ALS). In the brain, glia produce IGFBP; however the IGFBP-ALS has not been detected in the CNS. This gene represents the CNS equivalent of IGFBP-ALS. Because of the neuroprotective effects of IGF, therapeutic modulation of this gene or its protein product is useful in treating diseases in which neuronal death/degeneration occur such as amyotropic lateral sclerosis, multiple sclerosis, Alzheimer's disease, Parkinson's disease, Huntington's disease, spinocerebellar ataxia, or CNS injury such as stroke, head or spinal cord trauma.

Panel 2D Summary: Ag1094 Expression of this gene was highest in a metastatic breast cancer sample (CT=26-27). In addition, several other breast cancer and lung cancer samples showed increased expression when compared to their normal adjacent margin samples. This result was consistent with the results in the Ardais panel, and Panel 1.6 that both showed higher expression of this gene in cell lines and lung cancer tissue. Expression of this gene is useful as a marker to distinguish breast cancer or lung cancer tissue from their normal counterparts and is of diagnostic value. Therapeutic modulation of the this gene or its gene product, through the use of small molecule drugs or antibodies, is of benefit for treatment of breast or lung cancer.

Panel 3D Summary: Ag1094 Expression of this gene was highest in a small cell lung cancer cell line (CT=28.5). In addition, there was significant expression of this gene in other lung cancer cell lines as well as in several brain cancer and pancreatic cancer cell lines. These results were consistent with what is observed in the other panels. Expression of this gene is useful as a marker to distinguish lung, breast or pancreas cancer cell line samples from other tissues. Therapeutic modulation of this gene or gene product, through the use of small molecule drugs or antibodies, is of benefit for treatment of lung, breast or pancreatic cancer.

Panel 4.1D Summary: Ag6719/Ag6725 Highest expression of this gene was seen in activated basophils and resting keratinocytes (CTs=30-31). Significant expression of this gene was also seen in resting basophils, activated keratinocytes, activated NCI-H292, resting and activated lung fibroblasts. This expression pattern is in agreement with that seen in panel 4D. Please see panel 4D for further discussion on the utility of this gene. The protein encoded by this transcript is a homolog of insulin-like growth factor binding protein acid labile subunit, a component of the systemic insulin-like growth factor-binding protein (IGFBP) complex. This gene plays an important role in the biology of circulating IGFs. IGFs are involved in a wide array of cellular processes such as proliferation, prevention of apoptosis, and differentiation. This gene is a suitable target for a small molecule, antibody, or protein therapeutic to modulate locally the mitogenic effect of IGF and as a treatment of emphysema, COPD, or skin related disease.

AC. CG55117-04: Prominin-like Protein 1 Precursor

Expression of gene CG55117-04 was assessed using the primer-probe set Ag819, described in Table ACA. Results of the RTQ-PCR runs are shown in Tables ACB, ACC, ACD, ACE and ACF.

TABLE ACA
Probe Name Ag819

| | | | | SEQ |
|---|---|---|---|---|
| | | | Start | ID |
| Primers | Sequences | Length | Position | No |
| Forward | 5′-ggtccaacagggctatcaat-3′ | 20 | 1077 | 1402 |
| Probe | TET-5′-ccaaaccacgactgtcgtagcaggta-3′- | 26 | 1128 | 1403 |
| | TAMRA | | | |
| Reverse | 5′-tggaattcaagacccttttga-3′ | 21 | 1154 | 1404 |

TABLE ACB
HASS Panel v1.0

| Tissue Name | A |
|---|---|
| MCF-7 C1 | 0.0 |
| MCF-7 C2 | 0.0 |
| MCF-7 C3 | 0.0 |
| MCF-7 C4 | 0.0 |
| MCF-7 C5 | 0.1 |
| MCF-7 C6 | 0.0 |
| MCF-7 C7 | 0.0 |
| MCF-7 C9 | 0.0 |
| MCF-7 C10 | 0.0 |
| MCF-7 C11 | 0.0 |
| MCF-7 C12 | 0.1 |
| MCF-7 C13 | 0.0 |
| MCF-7 C15 | 0.0 |
| MCF-7 C16 | 0.0 |
| MCF-7 C17 | 0.0 |
| T24 D1 | 0.0 |
| T24 D2 | 0.0 |
| T24 D3 | 0.1 |
| T24 D4 | 0.1 |
| T24 D5 | 0.1 |
| T24 D6 | 0.0 |
| T24 D7 | 0.0 |
| T24 D9 | 0.0 |
| T24 D10 | 0.0 |
| T24 D11 | 0.0 |
| T24 D12 | 0.0 |
| T24 D13 | 0.0 |
| T24 D15 | 0.0 |
| T24 D16 | 0.0 |
| T24 D17 | 0.0 |
| CAPaN B1 | 66.9 |
| CAPaN B2 | 50.7 |
| CAPaN B3 | 11.8 |
| CAPaN B4 | 42.3 |
| CAPaN B5 | 45.4 |
| CAPaN B6 | 73.7 |
| CAPaN B7 | 27.7 |
| CAPaN B8 | 17.6 |
| CAPaN B9 | 67.4 |
| CAPaN B10 | 100.0 |
| CAPaN B11 | 53.2 |
| CAPaN B12 | 55.9 |
| CAPaN B13 | 28.7 |
| CAPaN B14 | 15.3 |
| CAPaN B15 | 39.0 |
| CAPaN B16 | 44.8 |
| CAPaN B17 | 36.9 |
| U87-MG F1 (B) | 0.1 |
| U87-MG F2 | 0.0 |
| U87-MG F3 | 0.0 |
| U87-MG F4 | 0.0 |
| U87-MG F5 | 0.1 |
| U87-MG F6 | 0.0 |
| U87-MG F7 | 0.3 |
| U87-MG F8 | 0.2 |
| U87-MG F9 | 0.3 |
| U87-MG F10 | 0.1 |
| U87-MG F11 | 0.0 |
| U87-MG F12 | 0.0 |
| U87-MG F13 | 0.7 |
| U87-MG F14 | 0.0 |
| U87-MG F15 | 0.5 |
| U87-MG F16 | 0.1 |
| U87-MG F17 | 0.1 |
| LnCAP A1 | 0.0 |
| LnCAP A2 | 0.0 |
| LnCAP A3 | 0.0 |
| LnCAP A4 | 0.0 |
| LnCAP A5 | 0.1 |
| LnCAP A6 | 0.1 |
| LnCAP A7 | 0.0 |
| LnCAP A8 | 0.1 |
| LnCAP A9 | 0.0 |
| LnCAP A10 | 0.0 |
| LnCAP A11 | 0.0 |
| LnCAP A12 | 0.0 |
| LnCAP A13 | 0.0 |
| LnCAP A14 | 0.0 |
| LnCAP A15 | 0.0 |
| LnCAP A16 | 0.0 |
| LnCAP A17 | 0.0 |
| Primary Astrocytes | 37.6 |
| Primary Renal Proximal Tubule Epithelial cell A2 | 38.2 |
| Primary melanocytes A5 | 0.1 |
| 126443 - 341 medullo | 0.4 |
| 126444 - 487 medullo | 4.8 |
| 126445 - 425 medullo | 0.0 |
| 126446 - 690 medullo | 1.8 |
| 126447 - 54 adult glioma | 0.0 |
| 126448 - 245 adult glioma | 1.5 |
| 126449 - 317 adult glioma | 10.7 |
| 126450 - 212 glioma | 5.9 |
| 126451 - 456 glioma | 60.7 |

Column A - Rel. Exp. (%) Ag819, Run 268362651

TABLE ACC
Panel 1.2

| Tissue Name | A | B |
|---|---|---|
| Endothelial cells | 0.0 | 0.0 |
| Heart (Fetal) | 0.4 | 0.8 |
| Pancreas | 43.8 | 48.3 |
| Pancreatic ca. CAPAN 2 | 8.1 | 17.9 |
| Adrenal Gland | 0.2 | 0.2 |
| Thyroid | 11.8 | 12.9 |
| Salivary gland | 63.3 | 63.7 |
| Pituitary gland | 0.9 | 0.5 |
| Brain (fetal) | 37.1 | 41.5 |
| Brain (whole) | 4.6 | 6.3 |
| Brain (amygdala) | 1.5 | 2.3 |
| Brain (cerebellum) | 0.9 | 1.5 |
| Brain (hippocampus) | 3.4 | 4.0 |
| Brain (thalamus) | 1.9 | 2.6 |
| Cerebral Cortex | 1.2 | 2.7 |
| Spinal cord | 1.2 | 1.9 |
| glio/astro U87-MG | 0.0 | 0.0 |
| glio/astro U-118-MG | 0.0 | 0.0 |
| astrocytoma SW1783 | 0.0 | 0.0 |
| neuro*; met SK-N-AS | 0.3 | 0.1 |
| astrocytoma SF-539 | 0.0 | 0.0 |
| astrocytoma SNB-75 | 0.0 | 0.0 |
| glioma SNB-19 | 0.0 | 0.1 |
| glioma U251 | 0.0 | 0.1 |
| glioma SF-295 | 0.0 | 0.0 |
| Heart | 8.1 | 9.5 |
| Skeletal Muscle | 2.6 | 3.7 |
| Bone marrow | 0.6 | 1.2 |
| Thymus | 0.0 | 0.1 |
| Spleen | 0.5 | 0.0 |
| Lymph node | 1.4 | 0.2 |
| Colorectal Tissue | 0.3 | 1.8 |
| Stomach | 10.7 | 23.3 |
| Small intestine | 10.4 | 18.9 |
| Colon ca. SW480 | 0.0 | 0.0 |
| Colon ca.* SW620 (SW480 met) | 9.0 | 11.7 |
| Colon ca. HT29 | 32.5 | 40.9 |
| Colon ca. HCT-116 | 5.9 | 7.9 |
| Colon ca. CaCo-2 | 100.0 | 100.0 |
| Colon ca. Tissue (ODO3866) | 4.7 | 5.4 |
| Colon ca. HCC-2998 | 2.5 | 3.0 |
| Gastric ca.* (liver met) NCI-N87 | 0.0 | 0.2 |
| Bladder | 39.2 | 49.7 |
| Trachea | 29.7 | 34.4 |
| Kidney | 27.4 | 25.7 |
| Kidney (fetal) | 17.7 | 19.1 |
| Renal ca. 786-0 | 0.0 | 0.0 |
| Renal ca. A498 | 0.0 | 0.1 |
| Renal ca. RXF 393 | 0.0 | 0.0 |
| Renal ca. ACHN | 0.0 | 0.0 |
| Renal ca. UO-31 | 1.0 | 1.6 |
| Renal ca. TK-10 | 0.0 | 0.0 |
| Liver | 8.0 | 3.3 |
| Liver (fetal) | 2.8 | 2.7 |
| Liver ca. (hepatoblast) HepG2 | 12.8 | 20.2 |
| Lung | 5.7 | 4.2 |
| Lung (fetal) | 9.5 | 7.4 |
| Lung ca. (small cell) LX-1 | 39.0 | 33.4 |
| Lung ca. (small cell) NCI-H69 | 7.4 | 10.5 |
| Lung ca. (s. cell var.) SHP-77 | 0.5 | 0.6 |
| Lung ca. (large cell)NCI-H460 | 0.0 | 0.0 |
| Lung ca. (non-sm. cell) A549 | 0.0 | 0.1 |
| Lung ca. (non-s. cell) NCI-H23 | 0.0 | 0.0 |
| Lung ca. (non-s. cell) HOP-62 | 0.1 | 0.2 |
| Lung ca. (non-s. cl) NCI-H522 | 0.0 | 0.1 |
| Lung ca. (squam.) SW 900 | 0.6 | 0.8 |
| Lung ca. (squam.) NCI-H596 | 14.6 | 22.1 |
| Mammary gland | 33.0 | 46.3 |
| Breast ca.* (pl. ef) MCF-7 | 0.0 | 0.0 |
| Breast ca.* (pl. ef) MDA-MB-231 | 0.0 | 0.0 |
| Breast ca.* (pl. ef) T47D | 0.8 | 1.3 |
| Breast ca. BT-549 | 0.0 | 0.0 |
| Breast ca. MDA-N | 0.4 | 0.6 |
| Ovary | 4.6 | 0.2 |
| Ovarian ca. OVCAR-3 | 3.3 | 4.0 |
| Ovarian ca. OVCAR-4 | 27.9 | 54.0 |
| Ovarian ca. OVCAR-5 | 37.4 | 51.1 |
| Ovarian ca. OVCAR-8 | 0.0 | 0.1 |
| Ovarian ca. IGROV-1 | 3.2 | 5.5 |
| Ovarian ca. (ascites) SK-OV-3 | 0.0 | 0.0 |
| Uterus | 1.4 | 1.2 |
| Placenta | 23.2 | 22.5 |
| Prostate | 2.6 | 2.7 |
| Prostate ca.* (bone met) PC-3 | 0.0 | 0.0 |
| Testis | 19.8 | 21.9 |
| Melanoma Hs688(A).T | 1.7 | 0.0 |
| Melanoma* (met) Hs688(B).T | 0.7 | 0.0 |
| Melanoma UACC-62 | 1.8 | 1.7 |
| Melanoma M14 | 0.1 | 0.2 |
| Melanoma LOX IMVI | 0.0 | 0.0 |
| Melanoma* (met) SK-MEL-5 | 0.5 | 1.0 |

Column A - Rel. Exp. (%) Ag819, Run 118349021
Column B - Rel. Exp. (%) Ag819, Run 120989791

TABLE ACD
Panel 2D

| Tissue Name | A | B |
|---|---|---|
| Normal Colon | 17.0 | 20.7 |
| CC Well to Mod Diff (ODO3866) | 0.9 | 5.3 |
| CC Margin (ODO3866) | 9.5 | 6.0 |
| CC Gr.2 rectosigmoid (ODO3868) | 5.8 | 3.9 |
| CC Margin (ODO3868) | 0.0 | 0.2 |
| CC Mod Diff (ODO3920) | 0.7 | 0.9 |
| CC Margin (ODO3920) | 2.8 | 2.1 |
| CC Gr.2 ascend colon (ODO3921) | 26.2 | 37.4 |
| CC Margin (ODO3921) | 4.4 | 7.0 |
| CC from Partial Hepatectomy | 13.1 | 20.4 |
| (ODO4309) Mets | | |
| Liver Margin (ODO4309) | 0.1 | 0.2 |
| Colon mets to lung (OD04451-01) | 8.5 | 6.2 |
| Lung Margin (OD04451-02) | 2.1 | 1.7 |
| Normal Prostate 6546-1 | 1.2 | 0.3 |
| Prostate Cancer (OD04410) | 0.5 | 0.7 |
| Prostate Margin (OD04410) | 0.8 | 0.6 |
| Prostate Cancer (OD04720-01) | 0.4 | 0.4 |
| Prostate Margin (OD04720-02) | 2.0 | 2.0 |
| Normal Lung 061010 | 4.6 | 4.7 |
| Lung Met to Muscle (ODO4286) | 0.0 | 0.0 |
| Muscle Margin (ODO4286) | 0.2 | 0.3 |
| Lung Malignant Cancer (OD03126) | 8.7 | 6.5 |
| Lung Margin (OD03126) | 1.4 | 1.6 |
| Lung Cancer (OD04404) | 0.1 | 0.2 |
| Lung Margin (OD04404) | 3.1 | 1.2 |
| Lung Cancer (OD04565) | 0.2 | 0.1 |
| Lung Margin (OD04565) | 1.0 | 0.9 |
| Lung Cancer (OD04237-01) | 100.0 | 74.7 |
| Lung Margin (OD04237-02) | 1.7 | 1.5 |
| Ocular Mel Met to Liver (ODO4310) | 0.1 | 0.3 |
| Liver Margin (ODO4310) | 0.6 | 0.2 |
| Melanoma Mets to Lung (OD04321) | 14.8 | 12.2 |
| Lung Margin (OD04321) | 1.7 | 1.5 |
| Normal Kidney | 23.2 | 17.4 |
| Kidney Ca, Nuclear grade 2 (OD04338) | 4.2 | 5.1 |
| Kidney Margin (OD04338) | 8.0 | 11.3 |
| Kidney Ca Nuclear grade 1/2 (OD04339) | 65.5 | 69.3 |
| Kidney Margin (OD04339) | 6.7 | 6.0 |
| Kidney Ca, Clear cell type (OD04340) | 0.1 | 0.1 |
| Kidney Margin (OD04340) | 13.8 | 12.0 |
| Kidney Ca, Nuclear grade 3 (OD04348) | 0.0 | 0.0 |
| Kidney Margin (OD04348) | 9.2 | 6.3 |
| Kidney Cancer (OD04622-01) | 0.7 | 0.4 |
| Kidney Margin (OD04622-03) | 1.1 | 1.2 |
| Kidney Cancer (OD04450-01) | 32.5 | 24.5 |
| Kidney Margin (OD04450-03) | 22.1 | 16.0 |
| Kidney Cancer 8120607 | 4.4 | 4.2 |
| Kidney Margin 8120608 | 3.9 | 2.0 |
| Kidney Cancer 8120613 | 0.1 | 0.0 |
| Kidney Margin 8120614 | 1.2 | 0.8 |
| Kidney Cancer 9010320 | 7.7 | 8.0 |
| Kidney Margin 9010321 | 7.8 | 6.0 |
| Normal Uterus | 0.0 | 0.0 |
| Uterus Cancer 064011 | 24.1 | 18.8 |
| Normal Thyroid | 4.7 | 2.4 |
| Thyroid Cancer 064010 | 4.0 | 2.2 |
| Thyroid Cancer A302152 | 0.1 | 0.0 |
| Thyroid Margin A302153 | 2.9 | 2.7 |
| Normal Breast | 16.6 | 7.4 |
| Breast Cancer (OD04566) | 0.6 | 0.4 |
| Breast Cancer (OD04590-01) | 0.8 | 0.5 |
| Breast Cancer Mets (OD04590-03) | 0.0 | 0.0 |
| Breast Cancer Metastasis (OD04655-05) | 0.1 | 0.0 |
| Breast Cancer 064006 | 15.7 | 11.7 |
| Breast Cancer 1024 | 12.1 | 11.6 |
| Breast Cancer 9100266 | 1.2 | 0.6 |
| Breast Margin 9100265 | 3.0 | 2.0 |
| Breast Cancer A209073 | 6.5 | 4.6 |
| Breast Margin A209073 | 25.0 | 9.0 |
| Normal Liver | 0.6 | 0.5 |
| Liver Cancer 064003 | 0.0 | 0.0 |
| Liver Cancer 1025 | 0.2 | 0.3 |
| Liver Cancer 1026 | 0.2 | 0.1 |
| Liver Cancer 6004-T | 0.2 | 0.1 |
| Liver Tissue 6004-N | 1.6 | 1.7 |
| Liver Cancer 6005-T | 0.1 | 0.2 |
| Liver Tissue 6005-N | 0.0 | 0.2 |
| Normal Bladder | 14.8 | 18.7 |
| Bladder Cancer 1023 | 6.9 | 6.4 |
| Bladder Cancer A302173 | 0.2 | 0.1 |
| Bladder Cancer (OD04718-01) | 0.1 | 0.1 |
| Bladder Normal Adjacent (OD04718-03) | 0.2 | 0.4 |
| Normal Ovary | 0.1 | 0.0 |
| Ovarian Cancer 064008 | 68.8 | 100.0 |
| Ovarian Cancer (OD04768-07) | 0.5 | 1.0 |
| Ovary Margin (OD04768-08) | 0.0 | 0.1 |
| Normal Stomach | 5.0 | 4.5 |
| Gastric Cancer 9060358 | 2.5 | 2.6 |
| Stomach Margin 9060359 | 5.6 | 7.0 |
| Gastric Cancer 9060395 | 1.7 | 1.3 |
| Stomach Margin 9060394 | 3.4 | 6.4 |
| Gastric Cancer 9060397 | 26.1 | 39.8 |
| Stomach Margin 9060396 | 2.7 | 2.7 |
| Gastric Cancer 064005 | 15.5 | 22.1 |

Column A - Rel. Exp. (%) Ag819, Run 144794769
Column B - Rel. Exp. (%) Ag819, Run 146791894

TABLE ACE
Panel 3D

| Tissue Name | A |
|---|---|
| Daoy- Medulloblastoma | 0.9 |
| TE671- Medulloblastoma | 1.1 |
| D283 Med- Medulloblastoma | 100.0 |
| PFSK-1- Primitive Neuroectodermal | 0.0 |
| XF-498- CNS | 0.1 |
| SNB-78- Glioma | 0.0 |
| SF-268- Glioblastoma | 0.0 |
| T98G- Glioblastoma | 0.0 |
| SK-N-SH- Neuroblastoma (metastasis) | 0.0 |
| SF-295- Glioblastoma | 0.0 |
| Cerebellum | 0.8 |
| Cerebellum | 0.2 |
| NCI-H292- Mucoepidermoid lung carcinoma | 0.0 |
| DMS-114- Small cell lung cancer | 0.1 |
| DMS-79- Small cell lung cancer | 9.9 |
| NCI-H146- Small cell lung cancer | 1.2 |
| NCI-H526- Small cell lung cancer | 0.0 |
| NCI-N417- Small cell lung cancer | 0.0 |
| NCI-H82- Small cell lung cancer | 28.5 |
| NCI-H157- Squamous cell lung cancer (metastasis) | 0.0 |
| NCI-H1155- Large cell lung cancer | 3.4 |
| NCI-H1299- Large cell lung cancer | 0.0 |
| NCI-H727- Lung carcinoid | 0.4 |
| NCI-UMC-11- Lung carcinoid | 10.4 |
| LX-1- Small cell lung cancer | 27.9 |
| Colo-205- Colon cancer | 3.0 |
| KM12- Colon cancer | 3.3 |
| KM20L2- Colon cancer | 1.2 |
| NCI-H716- Colon cancer | 0.0 |
| SW-48- Colon adenocarcinoma | 7.3 |
| SW1116- Colon adenocarcinoma | 3.7 |
| LS 174T- Colon adenocarcinoma | 0.2 |
| SW-948- Colon adenocarcinoma | 0.0 |
| SW-480- Colon adenocarcinoma | 3.1 |
| NCI-SNU-5- Gastric carcinoma | 0.0 |
| KATO III- Gastric carcinoma | 0.0 |
| NCI-SNU-16- Gastric carcinoma | 0.0 |
| NCI-SNU-1- Gastric carcinoma | 26.6 |
| RF-1- Gastric adenocarcinoma | 0.0 |
| RF-48- Gastric adenocarcinoma | 0.1 |
| MKN-45- Gastric carcinoma | 0.0 |
| NCI-N87- Gastric carcinoma | 0.0 |
| OVCAR-5- Ovarian carcinoma | 0.0 |
| RL95-2- Uterine carcinoma | 0.0 |
| HelaS3- Cervical adenocarcinoma | 0.0 |
| Ca Ski- Cervical epidermoid carcinoma (metastasis) | 0.0 |
| ES-2- Ovarian clear cell carcinoma | 0.0 |
| Ramos- Stimulated with PMA/ionomycin 6 h | 0.0 |
| Ramos- Stimulated with PMA/ionomycin 14 h | 0.0 |
| MEG-01- Chronic myelogenous leukemia (megokaryoblast) | 0.0 |
| Raji- Burkitt's lymphoma | 0.0 |
| Daudi- Burkitt's lymphoma | 0.0 |
| U266- B-cell plasmacytoma | 0.0 |
| CA46- Burkitt's lymphoma | 0.0 |
| RL- non-Hodgkin's B-cell lymphoma | 0.0 |
| JM1- pre-B-cell lymphoma | 0.0 |
| Jurkat- T cell leukemia | 0.0 |
| TF-1- Erythroleukemia | 0.0 |
| HUT 78- T-cell lymphoma | 0.0 |
| U937- Histiocytic lymphoma | 0.0 |
| KU-812- Myelogenous leukemia | 0.0 |
| 769-P- Clear cell renal carcinoma | 0.0 |
| Caki-2- Clear cell renal carcinoma | 0.0 |
| SW 839- Clear cell renal carcinoma | 0.0 |
| Rhabdoid kidney tumor | 0.0 |
| Hs766T- Pancreatic carcinoma (LN metastasis) | 0.0 |
| CAPAN-1- Pancreatic adenocarcinoma (liver metastasis) | 9.0 |
| SU86.86- Pancreatic carcinoma (liver metastasis) | 17.6 |
| BxPC-3- Pancreatic adenocarcinoma | 0.0 |
| HPAC- Pancreatic adenocarcinoma | 1.7 |
| MIA PaCa-2- Pancreatic carcinoma | 0.0 |
| CFPAC-1- Pancreatic ductal adenocarcinoma | 58.2 |
| PANC-1- Pancreatic epithelioid ductal carcinoma | 0.0 |
| T24- Bladder carcinoma (transitional cell) | 0.0 |
| 5637- Bladder carcinoma | 0.0 |
| HT-1197- Bladder carcinoma | 0.0 |
| UM-UC-3- Bladder carcinoma (transitional cell) | 0.0 |
| A204- Rhabdomyosarcoma | 0.0 |
| HT-1080- Fibrosarcoma | 0.0 |
| MG-63- Osteosarcoma | 0.0 |
| SK-LMS-1- Leiomyosarcoma (vulva) | 0.0 |
| SJRH30- Rhabdomyosarcoma (met to bone marrow) | 2.5 |
| A431- Epidermoid carcinoma | 0.0 |
| WM266-4- Melanoma | 0.2 |
| DU 145- Prostate carcinoma (brain metastasis) | 0.0 |
| MDA-MB-468- Breast adenocarcinoma | 11.2 |
| SCC-4- Squamous cell carcinoma of tongue | 0.0 |
| SCC-9- Squamous cell carcinoma of tongue | 0.0 |
| SCC-15- Squamous cell carcinoma of tongue | 0.0 |
| CAL 27- Squamous cell carcinoma of tongue | 0.0 |

Column A - Rel. Exp. (%) Ag819, Run 172133330

TABLE ACF
Panel 5 Islet

| Tissue Name | A |
|---|---|
| 97457_Patient-02go_adipose | 0.0 |
| 97476_Patient-07sk_skeletal muscle | 0.5 |
| 97477_Patient-07ut_uterus | 0.0 |
| 97478_Patient-07pl_placenta | 12.4 |
| 99167_Bayer Patient 1 | 7.9 |
| 97482_Patient-08ut_uterus | 0.4 |
| 97483_Patient-08pl_placenta | 20.9 |
| 97486_Patient-09sk_skeletal muscle | 0.0 |
| 97487_Patient-09ut_uterus | 0.0 |
| 97488_Patient-09pl_placenta | 11.0 |
| 97492_Patient-10ut_uterus | 8.1 |
| 97493_Patient-10pl_placenta | 47.0 |
| 97495_Patient-11go_adipose | 0.0 |
| 97496_Patient-11sk_skeletal muscle | 1.2 |
| 97497_Patient-11ut_uterus | 1.5 |
| 97498_Patient-11pl_placenta | 5.4 |
| 97500_Patient-12go_adipose | 0.0 |
| 97501_Patient-12sk_skeletal muscle | 1.1 |
| 97502_Patient-12ut_uterus | 0.0 |
| 97503_Patient-12pl_placenta | 11.7 |
| 94721_Donor 2 U - A_Mesenchymal Stem Cells | 0.0 |
| 94722_Donor 2 U - B_Mesenchymal Stem Cells | 0.0 |
| 94723_Donor 2 U - C_Mesenchymal Stem Cells | 0.0 |
| 94709_Donor 2 AM - A_adipose | 0.0 |
| 94710_Donor 2 AM - B_adipose | 0.0 |
| 94711_Donor 2 AM - C_adipose | 0.0 |
| 94712_Donor 2 AD - A_adipose | 0.0 |
| 94713_Donor 2 AD - B_adipose | 0.3 |
| 94714_Donor 2 AD - C_adipose | 0.0 |
| 94742_Donor 3 U - A_Mesenchymal Stem Cells | 0.0 |
| 94743_Donor 3 U - B_Mesenchymal Stem Cells | 0.0 |
| 94730_Donor 3 AM - A_adipose | 0.0 |
| 94731_Donor 3 AM - B_adipose | 0.0 |
| 94732_Donor 3 AM - C_adipose | 0.0 |
| 94733_Donor 3 AD - A_adipose | 0.0 |
| 94734_Donor 3 AD - B_adipose | 0.0 |
| 94735_Donor 3 AD - C_adipose | 0.0 |
| 77138_Liver_HepG2untreated | 100.0 |
| 73556_Heart_Cardiac stromal cells (primary) | 0.0 |
| 81735_Small Intestine | 38.2 |
| 72409_Kidney_Proximal Convoluted Tubule | 4.9 |
| 82685_Small intestine_Duodenum | 1.0 |
| 90650_Adrenal_Adrenocortical adenoma | 0.0 |
| 72410_Kidney_HRCE | 39.5 |
| 72411_Kidney_HRE | 97.9 |
| 73139_Uterus_Uterine smooth muscle cells | 0.0 |

Column A - Rel. Exp. (%) Ag819, Run 229431867

HASS Panel v1.0 Summary: Ag819 Highest expression of this gene was detected in the pancreatic cancer CaPaN cell lines subjected to acidosis (CT=27.9). Moderate levels of expression of this gene were seen in control and in CaPaN cells subjected to environmental stress. The expression of this gene was not significantly changed by oxygen deprivation, acidic or a serum starved environment in these cells. However, moderate to low expression was also shown in astrocytes, Renal Proximal Tubule Epithelial cell A2, ⅘ glioma and 2/4 medulloblastoma tissue samples in this panel. Therapeutic modulation of this gene, expressed protein and/or use of antibodies or small molecule drugs is useful in the treatment of pancreatic and brain cancers.

Panel 1.2 Summary: Ag819 The expression of this gene was highest in a colon cancer cell line (CaCo-2)(CTs=25). In addition, significant expression of this gene wais also seen in colon, ovarian, lung, pancreatic and a renal cancer cell lines. The expression of this gene is useful as a marker to detect the presence of these cancers and therapeutic modulation of this gene, expressed protein and/or use of antibodies or small molecule drug is useful in the treatment of these cancers.

Among tissues with metabolic function, this gene had moderate levels of expression in pancreas, adrenal gland, thyroid, pituitary gland, skeletal muscle, and adult and fetal liver. Modulation of this gene and/or expressed protein is useful in the treatment of diseases of the metabolic-endocrine axis, including obesity and Types 1 and 2 diabetes. In addition, this gene was differentially expressed in adult (CT value=28) vs fetal heart (CT value=32-33), and targeting this gene or gene product with small molecule, antibody, or protein therapeutics is of use in the treatment of heart related disorders.

This gene also showed moderate to low expression in all CNS regions examined, with highest expression in the fetal brain (greater than 10-fold) demonstrating a role in neurodevelopment. This gene codes for a Prominin like protein. Prominin plays a role in the formation of lipid membrane protrusions, their lipid content and membrane to membrane interactions; all critical for synapse formation. The expression of this gene in the developing brain shows a role in synaptogenesis for this molecule. Compensatory synaptogenesis has been shown to occur in the adult brain, especially in response to brain injury or neuronal loss. Therapeutic modulation of this gene and/or expressed protein is useful in the treatment of neurological disorders including stroke, head trauma, spinal cord trauma, Alzheimer's, Parkinson's or Huntington's diseases, multiple sclerosis, or ALS.

Panel 2D Summary: Ag819 Highest expression of this gene was detected in samples derived from an ovarian cancer and a lung cancer (CTs=26). Significant expression of this gene was seen in a number of cancer samples including a kidney cancer, two colon cancers and two gastric cancers. The expression of this gene is useful as a marker to detect the presence of ovarian or lung cancers. Therapeutic modulation of this gene, expressed protein and/or use of antibodies or small molecule drugs is of benefit in the treatment of ovarian, lung, kidney, colon or gastric cancers.

Panel 3D Summary: Ag819 The expression of this gene was highest in a sample derived from a brain cancer (meduloblastoma) cell line (D283 cells)(CT=28.3). Significant expression of this gene was also seen in several cell lines derived from lung, gastric and pancreatic cancers. Expression of this gene is useful as a marker to detect these cancers and therapeutic modulation of this gene, expressed protein, and/or use of antibodies or small molecule drugs targeting this gene or gene product is useful in the treatment of lung, brain, pancreatic or gastric cancers.

Panel 5 Islet Summary: Ag819 Highest expression of this gene was detected in liver cancer HepG2 cell line (CT=31). Low levels of expression of this gene are also seen in islets cells (Bayer patient 1), small intestine, kidney and placenta. Therefore, modulation of this gene and/or expressed protein is useful in treatment of metabolic diseases including obesity and type II diabetes.

AD. CG55193-04: PV1 Protein

Expression of gene CG55193-04 was assessed using the primer-probe sets Ag1537 and Ag4932, described in Tables ADA and ADB. Results of the RTQ-PCR runs are shown in Tables ADD, ADE, ADF, ADG, ADH and ADI.

TABLE ADA
Probe Name Ag1537

| | | | | SEQ |
|---|---|---|---|---|
| | | | Start | ID |
| Primers | Sequences | Length | Position | No |
| Forward | 5′-aaggagctggaagagaagaaga-3′ | 22 | 1087 | 1405 |
| Probe | TET-5′-atcagaaactcagccctggacacctg-3′- | 26 | 1141 | 1406 |
| | TAMRA | | | |
| Reverse | 5′-gctgcgacttggtcttgat-3′ | 19 | 1168 | 1407 |

TABLE ADB
Probe Name Ag4932

| | | | | SEQ |
|---|---|---|---|---|
| | | | Start | ID |
| Primers | Sequences | Length | Position | No |
| Forward | 5′-aatgcagagatcaattcaagga-3′ | 22 | 425 | 1408 |
| Probe | TET-5′-aacaagagctgcgatgccttgctctt-3′- | 26 | 451 | 1409 |
| | TAMRA | | | |
| Reverse | 5′-tcttcaccttctgattcagcat-3′ | 22 | 478 | 1410 |

TABLE ADC
Ardais Kidney 1.0

| Tissue Name | A |
|---|---|
| Kidney cancer(10A8) | 2.3 |
| Kidney NAT(10A9) | 6.9 |
| Kidney cancer(10AA) | 100.0 |
| Kidney NAT(10AB) | 7.4 |
| Kidney cancer(10AC) | 25.7 |
| Kidney NAT(10AD) | 5.3 |
| Kidney cancer(10B6) | 8.7 |
| Kidney NAT(10B7) | 4.5 |
| Kidney cancer(10B8) | 2.2 |
| Kidney NAT(10B9) | 10.6 |
| Kidney cancer(10BC) | 2.0 |
| Kidney NAT(10BD) | 20.2 |
| Kidney cancer(10BE) | 23.5 |
| Kidney NAT(10BF) | 9.7 |
| Kidney cancer(10C2) | 1.1 |
| Kidney NAT(10C3) | 9.1 |
| Kidney cancer(10C4) | 14.4 |
| Kidney NAT(10C5) | 9.4 |
| Kidney cancer(10B4) | 2.2 |
| Kidney cancer(10C8) | 14.5 |
| Kidney cancer(10D0) | 60.3 |
| Kidney cancer(10C0) | 1.2 |
| Kidney cancer(10C6) | 3.3 |
| Kidney cancer(10C9) | 1.5 |
| Kidney cancer(10D1) | 85.3 |
| Kidney cancer(10CA) | 0.8 |
| Kidney cancer(10D2) | 55.1 |
| Kidney cancer(10CB) | 6.4 |
| Kidney cancer(10D4) | 22.1 |
| Kidney cancer(10CD) | 73.2 |
| Kidney cancer(10D5) | 7.4 |
| Kidney cancer(10CE) | 63.3 |
| Kidney cancer(10D6) | 38.2 |
| Kidney cancer(10CF) | 22.8 |
| Kidney cancer(10D8) | 25.7 |
| Kidney cancer(10CC) | 28.1 |
| Kidney cancer(10D3) | 12.0 |
| Kidney NAT(10D9) | 7.4 |
| Kidney NAT(10DB) | 7.2 |
| Kidney NAT(10DC) | 10.8 |
| Kidney NAT(10DD) | 12.9 |
| Kidney NAT(10DE) | 20.7 |
| Kidney NAT(10B1) | 7.5 |
| Kidney NAT(10DA) | 7.5 |

Column A - Rel. Exp. (%) Ag1537, Run 385860665

TABLE ADD
Ardais Panel v.1.0

| Tissue Name | A |
|---|---|
| 136799_Lung cancer(362) | 23.8 |
| 136800_Lung NAT(363) | 15.6 |
| 136813_Lung cancer(372) | 45.4 |
| 136814_Lung NAT(373) | 14.4 |
| 136815_Lung cancer(374) | 39.2 |
| 136816_Lung NAT(375) | 100.0 |
| 136791_Lung cancer(35A) | 22.5 |
| 136795_Lung cancer(35E) | 35.4 |
| 136797_Lung cancer(360) | 22.4 |
| 136794_lung NAT(35D) | 14.3 |
| 136818_Lung NAT(377) | 33.0 |
| 136787_lung cancer(356) | 8.1 |
| 136788_lung NAT(357) | 52.5 |
| 136806_Lung cancer(36B) | 35.6 |
| 136807_Lung NAT(36C) | 18.8 |
| 136789_lung cancer(358) | 65.1 |
| 136802_Lung cancer(365) | 49.3 |
| 136803_Lung cancer(368) | 24.5 |
| 136804_Lung cancer(369) | 38.2 |
| 136811_Lung cancer(370) | 14.9 |
| 136810_Lung NAT(36F) | 31.4 |

Column A - Rel. Exp. (%) Ag1537, Run 267680189

TABLE ADE
General_screening_panel_v1.5

| Tissue Name | A |
|---|---|
| Adipose | 0.0 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 10.7 |
| Prostate ca.* (bone met) PC-3 | 0.0 |
| Prostate Pool | 14.7 |
| Placenta | 42.6 |
| Uterus Pool | 53.6 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 0.2 |
| Ovarian ca. IGROV-1 | 0.0 |
| Ovarian ca. OVCAR-8 | 0.1 |
| Ovary | 11.1 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 62.4 |
| Trachea | 47.3 |
| Lung | 4.3 |
| Fetal Lung | 17.2 |
| Lung ca. NCI-N417 | 0.0 |
| Lung ca. LX-1 | 0.0 |
| Lung ca. NCI-H146 | 0.0 |
| Lung ca. SHP-77 | 0.0 |
| Lung ca. A549 | 0.0 |
| Lung ca. NCI-H526 | 0.0 |
| Lung ca. NCI-H23 | 0.0 |
| Lung ca. NCI-H460 | 0.0 |
| Lung ca. HOP-62 | 0.0 |
| Lung ca. NCI-H522 | 0.0 |
| Liver | 1.2 |
| Fetal Liver | 44.1 |
| Liver ca. HepG2 | 0.0 |
| Kidney Pool | 55.1 |
| Fetal Kidney | 73.2 |
| Renal ca. 786-0 | 0.0 |
| Renal ca. A498 | 0.1 |
| Renal ca. ACHN | 0.0 |
| Renal ca. UO-31 | 0.0 |
| Renal ca. TK-10 | 0.0 |
| Bladder | 86.5 |
| Gastric ca. (liver met.) NCI-N87 | 4.5 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 51.1 |
| Colon ca. SW1116 | 0.0 |
| Colon ca. Colo-205 | 0.0 |
| Colon ca. SW-48 | 0.0 |
| Colon Pool | 89.5 |
| Small Intestine Pool | 11.7 |
| Stomach Pool | 37.9 |
| Bone Marrow Pool | 46.0 |
| Fetal Heart | 15.1 |
| Heart Pool | 22.2 |
| Lymph Node Pool | 66.9 |
| Fetal Skeletal Muscle | 23.2 |
| Skeletal Muscle Pool | 32.5 |
| Spleen Pool | 100.0 |
| Thymus Pool | 30.6 |
| CNS cancer (glio/astro) U87-MG | 0.0 |
| CNS cancer (glio/astro) U-118-MG | 0.1 |
| CNS cancer (neuro; met) SK-N-AS | 0.1 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.7 |
| Brain (Amygdala) Pool | 1.0 |
| Brain (cerebellum) | 2.0 |
| Brain (fetal) | 2.6 |
| Brain (Hippocampus) Pool | 2.1 |
| Cerebral Cortex Pool | 1.3 |
| Brain (Substantia nigra) Pool | 1.5 |
| Brain (Thalamus) Pool | 2.5 |
| Brain (whole) | 3.8 |
| Spinal Cord Pool | 1.9 |
| Adrenal Gland | 55.1 |
| Pituitary gland Pool | 10.3 |
| Salivary Gland | 20.7 |
| Thyroid (female) | 70.7 |
| Pancreatic ca. CAPAN2 | 0.0 |
| Pancreas Pool | 53.6 |

Column A - Rel. Exp. (%) Ag4932, Run 228843451

TABLE ADF
Oncology_cell_line_screening_panel_v3.2

| Tissue Name | A |
|---|---|
| 94905_Daoy_Medulloblastoma/Cerebellum_sscDNA | 0.0 |
| 94906_TE671_Medulloblastom/Cerebellum_sscDNA | 0.0 |
| 94907_D283 | 0.0 |
| Med_Medulloblastoma/Cerebellum_sscDNA | |
| 94908_PFSK-1_Primitive | 0.5 |
| Neuroectodermal/Cerebellum_sscDNA | |
| 94909_XF-498_CNS_sscDNA | 0.0 |
| 94910_SNB-78_CNS/glioma_sscDNA | 0.0 |
| 94911_SF-268_CNS/glioblastoma_sscDNA | 0.0 |
| 94912_T98G_Glioblastoma_sscDNA | 0.0 |
| 96776_SK-N-SH_Neuroblastoma (metastasis)_sscDNA | 0.0 |
| 94913_SF-295_CNS/glioblastoma_sscDNA | 0.0 |
| 132565_NT2 pool_sscDNA | 0.1 |
| 94914_Cerebellum_sscDNA | 0.2 |
| 96777_Cerebellum_sscDNA | 0.3 |
| 94916_NCI-H292_Mucoepidermoid lung carcinoma_sscDNA | 0.0 |
| 94917_DMS-114_Small cell lung cancer_sscDNA | 0.0 |
| 94918_DMS-79_Small cell lung | 0.0 |
| cancer/neuroendocrine_sscDNA | |
| 94919_NCI-H146_Small cell | 0.0 |
| lung cancer/neuroendocrine_sscDNA | |
| 94920_NCI-H526_Small cell | 0.0 |
| lung cancer/neuroendocrine_sscDNA | |
| 94921_NCI-N417_Small cell | 0.0 |
| lung cancer/neuroendocrine_sscDNA | |
| 94923_NCI-H82_Small cell lung | 0.0 |
| cancer/neuroendocrine_sscDNA | |
| 94924_NCI-H157_Squamous cell lung cancer | 0.0 |
| (metastasis)_sscDNA | |
| 94925_NCI-H1155_Large cell lung | 0.0 |
| cancer/neuroendocrine_sscDNA | |
| 94926_NCI-H1299_Large cell lung | 0.0 |
| cancer/neuroendocrine_sscDNA | |
| 94927_NCI-H727_Lung carcinoid_sscDNA | 0.0 |
| 94928_NCI-UMC-11_Lung carcinoid_sscDNA | 0.0 |
| 94929_LX-1_Small cell lung cancer_sscDNA | 0.0 |
| 94930_Colo-205_Colon cancer_sscDNA | 0.0 |
| 94931_KM12_Colon cancer_sscDNA | 0.0 |
| 94932_KM20L2_Colon cancer_sscDNA | 0.0 |
| 94933_NCI-H716_Colon cancer_sscDNA | 0.0 |
| 94935_SW-48_Colon adenocarcinoma_sscDNA | 0.0 |
| 94936_SW1116_Colon | 0.0 |
| adenocarcinoma_sscDNA | |
| 94937_LS 174T_Colon adenocarcinoma_sscDNA | 0.0 |
| 94938_SW-948_Colon adenocarcinoma_sscDNA | 0.0 |
| 94939_SW-480_Colon adenocarcinoma_sscDNA | 0.0 |
| 94940_NCI-SNU-5_Gastric carcinoma_sscDNA | 0.0 |
| 112197_KATO III_Stomach_sscDNA | 0.0 |
| 94943_NCI-SNU-16_Gastric carcinoma_sscDNA | 0.0 |
| 94944_NCI-SNU-1_Gastric carcinoma_sscDNA | 0.0 |
| 94946_RF-1_Gastric adenocarcinoma_sscDNA | 0.0 |
| 94947_RF-48_Gastric adenocarcinoma_sscDNA | 0.1 |
| 96778_MKN-45_Gastric carcinoma_sscDNA | 0.0 |
| 94949_NCI-N87_Gastric carcinoma_sscDNA | 0.0 |
| 94951_OVCAR-5_Ovarian carcinoma_sscDNA | 0.0 |
| 94952_RL95-2_Uterine carcinoma_sscDNA | 0.0 |
| 94953_HelaS3_Cervical adenocarcinoma_sscDNA | 0.0 |
| 94954_Ca Ski_Cervical epidermoid carcinoma | 0.2 |
| (metastasis)_sscDNA | |
| 94955_ES-2_Ovarian clear cell carcinoma_sscDNA | 0.0 |
| 94957_Ramos/6 h stim_Stimulated with PMA/ionomycin | 0.0 |
| 6 h_sscDNA | |
| 94958_Ramos/14 h stim_Stimulated with PMA/ionomycin | 0.0 |
| 14 h_sscDNA | |
| 94962_MEG-01_Chronic myelogenous leukemia | 0.7 |
| (megokaryoblast)_sscDNA | |
| 94963_Raji_Burkitt's lymphoma_sscDNA | 0.0 |
| 94964_Daudi_Burkitt's lymphoma_sscDNA | 0.8 |
| 94965_U266_B-cell plasmacytoma/myeloma_sscDNA | 1.3 |
| 94968_CA46_Burkitt's lymphoma_sscDNA | 0.2 |
| 94970_RL_non-Hodgkin's B-cell lymphoma_sscDNA | 0.0 |
| 94972_JM1_pre-B-cell lymphoma/leukemia_sscDNA | 0.0 |
| 94973_Jurkat_T cell leukemia_sscDNA | 0.0 |
| 94974_TF-1_Erythroleukemia_sscDNA | 100.0 |
| 94975_HUT 78_T-cell lymphoma_sscDNA | 0.0 |
| 94977_U937_Histiocytic lymphoma_sscDNA | 0.0 |
| 94980_KU-812_Myelogenous leukemia_sscDNA | 28.9 |
| 94981_769-P_Clear cell renal carcinoma_sscDNA | 0.1 |
| 94983_Caki-2_Clear cell renal carcinoma_sscDNA | 0.0 |
| 94984_SW 839_Clear cell renal carcinoma_sscDNA | 0.0 |
| 94986_G401_Wilms' tumor_sscDNA | 0.0 |
| 126768_293 cells_sscDNA | 0.0 |
| 94987_Hs766T_Pancreatic carcinoma (LN | 0.6 |
| metastasis)_sscDNA | |
| 94988_CAPAN-1_Pancreatic adenocarcinoma (liver | 0.0 |
| metastasis)_sscDNA | |
| 94989_SU86.86_Pancreatic carcinoma (liver | 0.0 |
| metastasis)_sscDNA | |
| 94990_BxPC-3_Pancreatic adenocarcinoma_sscDNA | 0.0 |
| 94991_HPAC_Pancreatic adenocarcinoma_sscDNA | 0.0 |
| 94992_MIA PaCa-2_Pancreatic carcinoma_sscDNA | 0.0 |
| 94993_CFPAC-1_Pancreatic ductal | 0.1 |
| adenocarcinoma_sscDNA | |
| 94994_PANC-1_Pancreatic epithelioid ductal | 0.0 |
| carcinoma_sscDNA | |
| 94996_T24_Bladder carcinoma (transitional | 0.1 |
| cell)_sscDNA | |
| 94997_5637_Bladder carcinoma_sscDNA | 0.0 |
| 94998_HT-1197_Bladder carcinoma_sscDNA | 0.0 |
| 94999_UM-UC-3_Bladder carcinoma (transitional | 0.0 |
| cell)_sscDNA | |
| 95000_A204_Rhabdomyosarcoma_sscDNA | 0.0 |
| 95001_HT-1080_Fibrosarcoma_sscDNA | 0.0 |
| 95002_MG-63_Osteosarcoma (bone)_sscDNA | 0.0 |
| 95003_SK-LMS-1_Leiomyosarcoma (vulva)_sscDNA | 0.2 |
| 95004_SJRH30_Rhabdomyosarcoma (met to bone | 0.0 |
| marrow)_sscDNA | |
| 95005_A431_Epidermoid carcinoma_sscDNA | 0.0 |
| 95007_WM266-4_Melanoma_sscDNA | 0.0 |
| 112195_DU 145_Prostate_sscDNA | 0.0 |
| 95012_MDA-MB-468_Breast adenocarcinoma_sscDNA | 0.0 |
| 112196_SSC-4_Tongue_sscDNA | 0.0 |
| 112194_SSC-9_Tongue_sscDNA | 0.0 |
| 112191_SSC-15_Tongue_sscDNA | 0.0 |
| 95017_CAL 27_Squamous cell carcinoma of | 0.0 |
| tongue_sscDNA | |

Column A - Rel. Exp. (%) Ag1537, Run 267177741

TABLE ADG
Panel 2D

| Tissue Name | A |
|---|---|
| Normal Colon | 12.3 |
| CC Well to Mod Diff (ODO3866) | 10.7 |
| CC Margin (ODO3866) | 12.2 |
| CC Gr.2 rectosigmoid (ODO3868) | 3.2 |
| CC Margin (ODO3868) | 0.8 |
| CC Mod Diff (ODO3920) | 3.4 |
| CC Margin (ODO3920) | 2.2 |
| CC Gr.2 ascend colon (ODO3921) | 13.4 |
| CC Margin (ODO3921) | 5.8 |
| CC from Partial Hepatectomy (ODO4309) Mets | 9.6 |
| Liver Margin (ODO4309) | 0.6 |
| Colon mets to lung (OD04451-01) | 5.5 |
| Lung Margin (OD04451-02) | 0.8 |
| Normal Prostate 6546-1 | 14.1 |
| Prostate Cancer (OD04410) | 8.8 |
| Prostate Margin (OD04410) | 6.9 |
| Prostate Cancer (OD04720-01) | 3.1 |
| Prostate Margin (OD04720-02) | 10.3 |
| Normal Lung 061010 | 11.8 |
| Lung Met to Muscle (ODO4286) | 6.4 |
| Muscle Margin (ODO4286) | 9.9 |
| Lung Malignant Cancer (OD03126) | 19.3 |
| Lung Margin (OD03126) | 3.3 |
| Lung Cancer (OD04404) | 5.2 |
| Lung Margin (OD04404) | 25.3 |
| Lung Cancer (OD04565) | 3.4 |
| Lung Margin (OD04565) | 3.1 |
| Lung Cancer (OD04237-01) | 11.0 |
| Lung Margin (OD04237-02) | 18.2 |
| Ocular Mel Met to Liver (ODO4310) | 0.7 |
| Liver Margin (ODO4310) | 1.7 |
| Melanoma Mets to Lung (OD04321) | 3.9 |
| Lung Margin (OD04321) | 3.7 |
| Normal Kidney | 40.6 |
| Kidney Ca, Nuclear grade 2 (OD04338) | 5.7 |
| Kidney Margin (OD04338) | 11.1 |
| Kidney Ca Nuclear grade 1/2 (OD04339) | 2.5 |
| Kidney Margin (OD04339) | 17.6 |
| Kidney Ca, Clear cell type (OD04340) | 100.0 |
| Kidney Margin (OD04340) | 22.7 |
| Kidney Ca, Nuclear grade 3 (OD04348) | 55.1 |
| Kidney Margin (OD04348) | 19.9 |
| Kidney Cancer (OD04622-01) | 25.0 |
| Kidney Margin (OD04622-03) | 7.4 |
| Kidney Cancer (OD04450-01) | 1.3 |
| Kidney Margin (OD04450-03) | 9.2 |
| Kidney Cancer 8120607 | 9.2 |
| Kidney Margin 8120608 | 23.5 |
| Kidney Cancer 8120613 | 21.5 |
| Kidney Margin 8120614 | 12.3 |
| Kidney Cancer 9010320 | 34.4 |
| Kidney Margin 9010321 | 27.7 |
| Normal Uterus | 9.3 |
| Uterus Cancer 064011 | 6.4 |
| Normal Thyroid | 84.1 |
| Thyroid Cancer 064010 | 20.6 |
| Thyroid Cancer A302152 | 15.2 |
| Thyroid Margin A302153 | 21.3 |
| Normal Breast | 22.1 |
| Breast Cancer (OD04566) | 8.4 |
| Breast Cancer (OD04590-01) | 21.0 |
| Breast Cancer Mets (OD04590-03) | 27.7 |
| Breast Cancer Metastasis (OD04655-05) | 9.1 |
| Breast Cancer 064006 | 10.1 |
| Breast Cancer 1024 | 7.1 |
| Breast Cancer 9100266 | 10.4 |
| Breast Margin 9100265 | 7.4 |
| Breast Cancer A209073 | 27.4 |
| Breast Margin A209073 | 8.7 |
| Normal Liver | 1.1 |
| Liver Cancer 064003 | 6.5 |
| Liver Cancer 1025 | 0.7 |
| Liver Cancer 1026 | 8.1 |
| Liver Cancer 6004-T | 1.9 |
| Liver Tissue 6004-N | 3.6 |
| Liver Cancer 6005-T | 9.3 |
| Liver Tissue 6005-N | 0.6 |
| Normal Bladder | 14.1 |
| Bladder Cancer 1023 | 4.5 |
| Bladder Cancer A302173 | 3.6 |
| Bladder Cancer (OD04718-01) | 7.4 |
| Bladder Normal Adjacent (OD04718-03) | 15.2 |
| Normal Ovary | 1.4 |
| Ovarian Cancer 064008 | 6.5 |
| Ovarian Cancer (OD04768-07) | 1.6 |
| Ovary Margin (OD04768-08) | 9.2 |
| Normal Stomach | 13.5 |
| Gastric Cancer 9060358 | 2.8 |
| Stomach Margin 9060359 | 12.6 |
| Gastric Cancer 9060395 | 20.6 |
| Stomach Margin 9060394 | 7.5 |
| Gastric Cancer 9060397 | 10.0 |
| Stomach Margin 9060396 | 3.2 |
| Gastric Cancer 064005 | 6.7 |

Column A - Rel. Exp. (%) Ag1537, Run 145017308

TABLE ADH
Panel 4.1D

| Tissue Name | A |
|---|---|
| Secondary Th1 act | 0.1 |
| Secondary Th2 act | 0.4 |
| Secondary Tr1 act | 0.1 |
| Secondary Th1 rest | 0.1 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 1.2 |
| CD45RO CD4 lymphocyte act | 0.2 |
| CD8 lymphocyte act | 0.1 |
| Secondary CD8 lymphocyte rest | 0.4 |
| Secondary CD8 lymphocyte act | 0.0 |
| CD4 lymphocyte none | 0.3 |
| 2ry Th1/Th2/Tr1_anti-CD95 CH11 | 0.0 |
| LAK cells rest | 0.1 |
| LAK cells IL-2 | 0.1 |
| LAK cells IL-2 + IL-12 | 0.2 |
| LAK cells IL-2 + IFN gamma | 0.5 |
| LAK cells IL-2 + IL-18 | 0.2 |
| LAK cells PMA/ionomycin | 0.2 |
| NK Cells IL-2 rest | 0.2 |
| Two Way MLR 3 day | 2.7 |
| Two Way MLR 5 day | 1.3 |
| Two Way MLR 7 day | 0.1 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.1 |
| B lymphocytes PWM | 0.0 |
| B lymphocytes CD40L and IL-4 | 0.5 |
| EOL-1 dbcAMP | 0.0 |
| EOL-1 dbcAMP PMA/ionomycin | 0.1 |
| Dendritic cells none | 0.1 |
| Dendritic cells LPS | 1.7 |
| Dendritic cells anti-CD40 | 0.9 |
| Monocytes rest | 0.6 |
| Monocytes LPS | 0.1 |
| Macrophages rest | 0.0 |
| Macrophages LPS | 0.1 |
| HUVEC none | 1.9 |
| HUVEC starved | 8.4 |
| HUVEC IL-1beta | 5.6 |
| HUVEC IFN gamma | 40.6 |
| HUVEC TNF alpha + IFN gamma | 4.6 |
| HUVEC TNF alpha + IL4 | 5.0 |
| HUVEC IL-11 | 8.7 |
| Lung Microvascular EC none | 66.4 |
| Lung Microvascular EC TNFalpha + IL-1beta | 30.4 |
| Microvascular Dermal EC none | 43.5 |
| Microsvasular Dermal EC TNFalpha + IL-1beta | 17.0 |
| Bronchial epithelium TNFalpha + IL1beta | 0.3 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 1.1 |
| Astrocytes rest | 0.0 |
| Astrocytes TNFalpha + IL-1beta | 0.2 |
| KU-812 (Basophil) rest | 27.0 |
| KU-812 (Basophil) PMA/ionomycin | 28.3 |
| CCD1106 (Keratinocytes) none | 0.0 |
| CCD1106 (Keratinocytes) TNFalpha + IL-1beta | 0.0 |
| Liver cirrhosis | 20.6 |
| NCI-H292 none | 0.0 |
| NCI-H292 IL-4 | 0.0 |
| NCI-H292 IL-9 | 0.0 |
| NCI-H292 IL-13 | 0.1 |
| NCI-H292 IFN gamma | 0.0 |
| HPAEC none | 1.8 |
| HPAEC TNF alpha + IL-1 beta | 1.5 |
| Lung fibroblast none | 0.4 |
| Lung fibroblast TNF alpha + IL-1 beta | 0.6 |
| Lung fibroblast IL-4 | 0.2 |
| Lung fibroblast IL-9 | 0.0 |
| Lung fibroblast IL-13 | 0.4 |
| Lung fibroblast IFN gamma | 0.2 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 1.1 |
| Dermal fibroblast IL-4 | 0.4 |
| Dermal Fibroblasts rest | 0.7 |
| Neutrophils TNFa + LPS | 0.4 |
| Neutrophils rest | 0.3 |
| Colon | 19.3 |
| Lung | 100.0 |
| Thymus | 48.0 |
| Kidney | 68.8 |

Column A - Rel. Exp. (%) Ag4932, Run 223597251

TABLE ADI
general oncology screening panel_v_2.4

| Tissue Name | A |
|---|---|
| Colon cancer 1 | 11.4 |
| Colon cancer NAT 1 | 9.4 |
| Colon cancer 2 | 4.1 |
| Colon cancer NAT 2 | 4.1 |
| Colon cancer 3 | 10.4 |
| Colon cancer NAT 3 | 7.6 |
| Colon malignant cancer 4 | 9.7 |
| Colon normal adjacent tissue 4 | 3.4 |
| Lung cancer 1 | 4.8 |
| Lung NAT 1 | 0.7 |
| Lung cancer 2 | 5.6 |
| Lung NAT 2 | 0.1 |
| Squamous cell carcinoma 3 | 5.4 |
| Lung NAT 3 | 0.7 |
| metastatic melanoma 1 | 1.5 |
| Melanoma 2 | 4.0 |
| Melanoma 3 | 3.8 |
| metastatic melanoma 4 | 2.2 |
| metastatic melanoma 5 | 4.6 |
| Bladder cancer 1 | 1.0 |
| Bladder cancer NAT 1 | 0.0 |
| Bladder cancer 2 | 4.4 |
| Bladder cancer NAT 2 | 0.6 |
| Bladder cancer NAT 3 | 0.8 |
| Bladder cancer NAT 4 | 4.2 |
| Prostate adenocarcinoma 1 | 2.1 |
| Prostate adenocarcinoma 2 | 1.2 |
| Prostate adenocarcinoma 3 | 2.1 |
| Prostate adenocarcinoma 4 | 6.0 |
| Prostate cancer NAT 5 | 3.0 |
| Prostate adenocarcinoma 6 | 1.3 |
| Prostate adenocarcinoma 7 | 1.2 |
| Prostate adenocarcinoma 8 | 1.2 |
| Prostate adenocarcinoma 9 | 4.8 |
| Prostate cancer NAT 10 | 0.6 |
| Kidney cancer 1 | 90.1 |
| Kidney NAT 1 | 5.0 |
| Kidney cancer 2 | 60.3 |
| Kidney NAT 2 | 9.8 |
| Kidney cancer 3 | 30.8 |
| Kidney NAT 3 | 5.4 |
| Kidney cancer 4 | 100.0 |
| Kidney NAT 4 | 6.7 |

Column A - Rel. Exp. (%) Ag1537, Run 266930996

Ardais Kidney 1.0 Summary: Ag1537 Highest expression was seen in a kidney cancer sample (CT=21.3). In addition, this gene was more highly expressed in kidney cancer than in the corresponding normal adjacent tissue. Expression of this gene is useful as a marker of these cancers. Therapeutic modulation of the expression or function of this gene or gene product is useful in the treatment of kidney cancer.

Ardais Panel v.1.0 Summary: Ag1537 Highest expression of this gene was detected in a normal lung sample (CT=26.7). High to moderate levels of expression are seen in both malignant and normal lung samples. Therapeutic modulation of the PV1 protein (PLVAP) encoded by this gene is useful in the treatment of certain subtypes of lung cancer.

General_screening₁₃ panel_v1.5 Summary: Ag4932 Highest expression of this gene was detected in the spleen (CT=26). High levels of expression of this gene were also detected in tissues with metabolic/endocrine functions including pancreas, adipose, adrenal gland, thyroid, pituitary gland, skeletal muscle, heart, liver and the gastrointestinal tract. The PV-1-like protein is a plasma membrane protein with an extracellular domain that is an antibody target for the treatment of endocrine/metabolically related diseases, such as obesity and diabetes.

Moderate levels of expression of this gene were also seen in all regions of the central nervous system examined, including amygdala, hippocampus, substantia nigra, thalamus, cerebellum, cerebral cortex, and spinal cord. Therapeutic modulation of this gene or gene product is useful in the treatment of central nervous system disorders such as Alzheimer's disease, Parkinson's disease, epilepsy, multiple sclerosis, schizophrenia and depression.

In addition, this gene also showed high expression in colon cancer tissue, with moderate levels of expression in a gastric NCI-N87 cell line. Therapeutic modulation of this gene is useful in the treatment of colon and gastric cancers.

Oncology₁₃ cell₁₃ line_screening₁₃ panel_v3.2 Summary: Ag1537 Highest expression of this gene was detected in TF-1 erythroleukemia cells (CT=28.6). Moderate levels of expression of this gene are restricted to erythroleukemia and myelogenous leukemia. Expression of this gene is useful as a marker to distinguish these leukemia samples from other samples in the panel and also, as marker to detect the presence of these leukemia. Therapeutic modulation of this gene or its protein product is useful in the treatment of erythroleukemia and myelogenous leukemia.

Panel 2D Summary: Ag1537 Expression of this gene was highest in a kidney cancer (OD04340) sample (CT=25). This gene was widely expressed across this panel with high to moderate expression in both normal and adjacent cancer tissue. This gene was more highly expressed in kidney cancer tissue than in adjacent normal tissue, consistent with expression pattern seen in panel 2.4. This gene is useful as a marker to distinguish kidney cancers from normal kidney tissue. Therapeutic modulation of this gene or its protein product, through the use of small molecule drugs or antibodies, is of benefit in the treatment of kidney cancer.

Panel 4.1D Summary: Ag4932 Highest expression of this gene was detected in the lung (CT=28.5). Moderate levels of expression of this gene were also seen in endothelial cells, basophils and normal tissues represented by colon, thymus and kidney. This gene codes for a variant of PV-1, a component of the endothelial fenestral and stomatal diaphragms. Antibodies raised against the PV-1 encoded by this gene are useful in preventing transendothelial trafficking of inflammatory cells to different tissues sites and as a treatment of inflammatory diseases including delayed type hypersensitivity, asthma, emphysema, rheumatoid arthritis and IBD.

Moderate levels of expression of this gene were also seen in liver cirrhosis samples. Therefore, targeting this gene or its protein product with protein therapeutics, antibodies or small molecule therapeutics is useful as a treatment to reduce or inhibit fibrosis that occurs in liver cirrhosis.

general oncology screening panel_v_{—}2.4 Summary: Ag1537/Ag760 Highest expression of this gene was seen in a kidney cancer sample (CTs=22.6-25). Significant expression of this gene was seen in melanoma, colon, lung, prostate, bladder and kidney cancer as well as normal tissue samples. Expression of this gene was higher in kidney cancer as compared to corresponding normal control samples. Expression of this gene is useful as a marker to distinguish kidney cancer from normal tissue and also as a marker to detect kidney cancer. Therapeutic modulation of this gene or its protein product through the use of antibodies, protein therapeutics or small molecule drug is useful in the treatment of melanoma, kidney, colon, lung and prostate cancers.

AE. CG5525607: Apolipoprotein E Receptor 2

Expression of gene CG55256-07 was assessed using the primer-probe set Ag6829, described in Table AEA. Results of the RTQ-PCR runs are shown in Tables AEB and AEC.

TABLE AEA
Probe Name Ag6829

| | | | Start | SEQ ID |
|---|---|---|---|---|
| Primers | Sequences | Length | Position | No |
| Forward | 5′-aggacatgctgtggatgaag-3′ | 20 | 3987 | 1411 |
| Probe | TET-5′-tcacatctcttatttctcttttcccctt-3′- | 28 | 4031 | 1412 |
| | TAMRA | | | |
| Reverse | 5′-ccccacataaaggaaatggt-3′ | 20 | 4066 | 1413 |

TABLE AEB
General_screening_panel_v1.7

| Tissue Name | A |
|---|---|
| Adipose | 1.5 |
| HUVEC | 3.6 |
| Melanoma* Hs688(A).T | 0.0 |
| Melanoma* Hs688(B).T | 4.5 |
| Melanoma (met) SK-MEL-5 | 5.7 |
| Testis | 4.8 |
| Prostate ca. (bone met) PC-3 | 0.0 |
| Prostate ca. DU145 | 5.0 |
| Prostate pool | 0.1 |
| Uterus pool | 0.0 |
| Ovarian ca. OVCAR-3 | 1.9 |
| Ovarian ca. (ascites) SK-OV-3 | 0.1 |
| Ovarian ca. OVCAR-4 | 13.3 |
| Ovarian ca. OVCAR-5 | 13.2 |
| Ovarian ca. IGROV-1 | 29.9 |
| Ovarian ca. OVCAR-8 | 13.9 |
| Ovary | 0.4 |
| Breast ca. MCF-7 | 3.2 |
| Breast ca. MDA-MB-231 | 12.7 |
| Breast ca. BT-549 | 3.2 |
| Breast ca. T47D | 1.5 |
| Breast pool | 0.0 |
| Trachea | 1.4 |
| Lung | 1.3 |
| Fetal Lung | 1.4 |
| Lung ca. NCI-N417 | 2.2 |
| Lung ca. LX-1 | 1.9 |
| Lung ca. NCI-H146 | 3.8 |
| Lung ca. SHP-77 | 40.9 |
| Lung ca. NCI-H23 | 8.1 |
| Lung ca. NCI-H460 | 46.3 |
| Lung ca. HOP-62 | 15.3 |
| Lung ca. NCI-H522 | 9.2 |
| Lung ca. DMS-114 | 1.4 |
| Liver | 0.0 |
| Fetal Liver | 0.1 |
| Kidney pool | 0.8 |
| Fetal Kidney | 0.1 |
| Renal ca. 786-0 | 15.6 |
| Renal ca. A498 | 1.0 |
| Renal ca. ACHN | 4.2 |
| Renal ca. UO-31 | 1.1 |
| Renal ca. TK-10 | 7.6 |
| Bladder | 0.2 |
| Gastric ca. (liver met.) NCI-N87 | 1.3 |
| Stomach | 0.0 |
| Colon ca. SW-948 | 2.6 |
| Colon ca. SW480 | 1.1 |
| Colon ca. (SW480 met) SW620 | 14.4 |
| Colon ca. HT29 | 100.0 |
| Colon ca. HCT-116 | 30.6 |
| Colon cancer tissue | 0.0 |
| Colon ca. SW1116 | 7.6 |
| Colon ca. Colo-205 | 4.4 |
| Colon ca. SW-48 | 1.8 |
| Colon | 0.1 |
| Small Intestine | 0.0 |
| Fetal Heart | 0.0 |
| Heart | 0.0 |
| Lymph Node pool 1 | 0.0 |
| Lymph Node pool 2 | 3.3 |
| Fetal Skeletal Muscle | 0.1 |
| Skeletal Muscle pool | 0.0 |
| Skeletal Muscle | 0.0 |
| Spleen | 0.2 |
| Thymus | 0.1 |
| CNS cancer (glio/astro) SF-268 | 3.0 |
| CNS cancer (glio/astro) T98G | 5.7 |
| CNS cancer (neuro; met) SK-N-AS | 0.6 |
| CNS cancer (astro) SF-539 | 10.5 |
| CNS cancer (astro) SNB-75 | 9.3 |
| CNS cancer (glio) SNB-19 | 23.0 |
| CNS cancer (glio) SF-295 | 3.1 |
| Brain (Amygdala) | 3.4 |
| Brain (Cerebellum) | 4.5 |
| Brain (Fetal) | 24.0 |
| Brain (Hippocampus) | 1.9 |
| Cerebral Cortex pool | 2.5 |
| Brain (Substantia nigra) | 1.2 |
| Brain (Thalamus) | 3.3 |
| Brain (Whole) | 21.9 |
| Spinal Cord | 0.5 |
| Adrenal Gland | 0.3 |
| Pituitary Gland | 2.4 |
| Salivary Gland | 0.1 |
| Thyroid | 13.9 |
| Pancreatic ca. PANC-1 | 1.2 |
| Pancreas pool | 0.1 |

Column A - Rel. Exp. (%) Ag6829, Run 318350033

TABLE AEC
Panel 5 Islet

| Tissue Name | A |
|---|---|
| 97457_Patient-02go_adipose | 0.0 |
| 97476_Patient-07sk_skeletal muscle | 0.0 |
| 97477_Patient-07ut_uterus | 0.0 |
| 97478_Patient-07pl_placenta | 21.2 |
| 99167_Bayer Patient 1 | 2.4 |
| 97482_Patient-08ut_uterus | 0.0 |
| 97483_Patient-08pl_placenta | 10.7 |
| 97486_Patient-09sk_skeletal muscle | 0.0 |
| 97487_Patient-09ut_uterus | 0.0 |
| 97488_Patient-09pl_placenta | 12.2 |
| 97492_Patient-10ut_uterus | 0.0 |
| 97493_Patient-10pl_placenta | 63.7 |
| 97495_Patient-11go_adipose | 0.0 |
| 97496_Patient-11sk_skeletal muscle | 0.0 |
| 97497_Patient-11ut_uterus | 4.3 |
| 97498_Patient-11pl_placenta | 15.9 |
| 97500_Patient-12go_adipose | 0.0 |
| 97501_Patient-12sk_skeletal muscle | 2.9 |
| 97502_Patient-12ut_uterus | 5.4 |
| 97503_Patient-12pl_placenta | 62.4 |
| 94721_Donor 2 U - A_Mesenchymal Stem Cells | 7.4 |
| 94722_Donor 2 U - B_Mesenchymal Stem Cells | 5.9 |
| 94723_Donor 2 U - C_Mesenchymal Stem Cells | 6.7 |
| 94709_Donor 2 AM - A_adipose | 11.9 |
| 94710_Donor 2 AM - B_adipose | 5.0 |
| 94711_Donor 2 AM - C_adipose | 4.5 |
| 94712_Donor 2 AD - A_adipose | 7.5 |
| 94713_Donor 2 AD - B_adipose | 17.6 |
| 94714_Donor 2 AD - C_adipose | 8.4 |
| 94742_Donor 3 U - A_Mesenchymal Stem Cells | 0.0 |
| 94743_Donor 3 U - B_Mesenchymal Stem Cells | 0.0 |
| 94730_Donor 3 AM - A_adipose | 19.8 |
| 94731_Donor 3 AM - B_adipose | 37.9 |
| 94732_Donor 3 AM - C_adipose | 29.9 |
| 94733_Donor 3 AD - A_adipose | 32.5 |
| 94734_Donor 3 AD - B_adipose | 17.2 |
| 94735_Donor 3 AD - C_adipose | 18.9 |
| 77138_Liver_HepG2untreated | 100.0 |
| 73556_Heart_Cardiac stromal cells (primary) | 4.9 |
| 81735_Small Intestine | 2.6 |
| 72409_Kidney_Proximal Convoluted Tubule | 29.5 |
| 82685_Small intestine_Duodenum | 0.0 |
| 90650_Adrenal_Adrenocortical adenoma | 0.0 |
| 72410_Kidney_HRCE | 67.8 |
| 72411_Kidney_HRE | 37.6 |
| 73139_Uterus_Uterine smooth muscle cells | 15.7 |

Column A - Rel. Exp. (%) Ag6829, Run 279524965

General_screening₁₃ panel₁₃ v1.7 Summary: Ag6829 Highest expression was seen in a colon cancer cell line (CT=28). Prominent levels of expression were seen in clusters of cell lines derived from brain, colon, lung and ovarian cancer cell lines. Targeting this gene or its protein product is useful in the treatment of these cancers.

Panel 5 Islet Summary: Ag6829 Low but significant levels of expression were seen in a liver cell line (CT=34).

AF. CG5525610: Apolipoprotein E Receptor 2

Expression of full-length physical clone CG55256-10 was assessed using the primer-probe set Ag7103, described in Table AFA. Results of the RTQ-PCR runs are shown in Table AFB.

TABLE AFA
Probe Name Ag7103

| | | | Start | SEQ ID |
|---|---|---|---|---|
| Primers | Sequences | Length | Position | No |
| Forward | 5′-cccagcttctctgcacca-3′ | 18 | 778 | 1414 |
| Probe | TET-5′-cctcggactcatcggagccatca-3′- | 23 | 818 | 1415 |
| | TAMRA | | | |
| Reverse | 5′-cggcgaggaggagtgtc-3′ | 17 | 842 | 1416 |

TABLE AFB
General_screening_panel_v1.7

| Tissue Name | A |
|---|---|
| Adipose | 1.2 |
| HUVEC | 4.6 |
| Melanoma* Hs688(A).T | 0.0 |
| Melanoma* Hs688(B).T | 6.3 |
| Melanoma (met) SK-MEL-5 | 7.8 |
| Testis | 9.2 |
| Prostate ca. (bone met) PC-3 | 0.0 |
| Prostate ca. DU145 | 13.7 |
| Prostate pool | 0.1 |
| Uterus pool | 0.1 |
| Ovarian ca. OVCAR-3 | 0.9 |
| Ovarian ca. (ascites) SK-OV-3 | 0.7 |
| Ovarian ca. OVCAR-4 | 11.0 |
| Ovarian ca. OVCAR-5 | 10.4 |
| Ovarian ca. IGROV-1 | 19.6 |
| Ovarian ca. OVCAR-8 | 8.6 |
| Ovary | 0.5 |
| Breast ca. MCF-7 | 6.0 |
| Breast ca. MDA-MB-231 | 12.2 |
| Breast ca. BT-549 | 4.9 |
| Breast ca. T47D | 3.6 |
| Breast pool | 0.1 |
| Trachea | 1.3 |
| Lung | 1.0 |
| Fetal Lung | 1.3 |
| Lung ca. NCI-N417 | 7.2 |
| Lung ca. LX-1 | 6.7 |
| Lung ca. NCI-H146 | 5.9 |
| Lung ca. SHP-77 | 18.7 |
| Lung ca. NCI-H23 | 7.9 |
| Lung ca. NCI-H460 | 26.4 |
| Lung ca. HOP-62 | 9.9 |
| Lung ca. NCI-H522 | 7.1 |
| Lung ca. DMS-114 | 2.5 |
| Liver | 0.0 |
| Fetal Liver | 0.2 |
| Kidney pool | 0.5 |
| Fetal Kidney | 0.3 |
| Renal ca. 786-0 | 11.0 |
| Renal ca. A498 | 3.2 |
| Renal ca. ACHN | 4.3 |
| Renal ca. UO-31 | 1.7 |
| Renal ca. TK-10 | 9.9 |
| Bladder | 0.5 |
| Gastric ca. (liver met.) NCI-N87 | 2.6 |
| Stomach | 0.1 |
| Colon ca. SW-948 | 2.7 |
| Colon ca. SW480 | 4.9 |
| Colon ca. (SW480 met) SW620 | 15.5 |
| Colon ca. HT29 | 64.6 |
| Colon ca. HCT-116 | 24.0 |
| Colon cancer tissue | 0.2 |
| Colon ca. SW1116 | 19.1 |
| Colon ca. Colo-205 | 8.2 |
| Colon ca. SW-48 | 3.6 |
| Colon | 0.3 |
| Small Intestine | 0.0 |
| Fetal Heart | 0.1 |
| Heart | 0.1 |
| Lymph Node pool 1 | 0.1 |
| Lymph Node pool 2 | 2.2 |
| Fetal Skeletal Muscle | 0.3 |
| Skeletal Muscle pool | 0.0 |
| Skeletal Muscle | 0.1 |
| Spleen | 0.3 |
| Thymus | 0.5 |
| CNS cancer (glio/astro) SF-268 | 5.3 |
| CNS cancer (glio/astro) T98G | 8.4 |
| CNS cancer (neuro; met) SK-N-AS | 100.0 |
| CNS cancer (astro) SF-539 | 9.7 |
| CNS cancer (astro) SNB-75 | 10.2 |
| CNS cancer (glio) SNB-19 | 18.4 |
| CNS cancer (glio) SF-295 | 6.8 |
| Brain (Amygdala) | 5.0 |
| Brain (Cerebellum) | 11.3 |
| Brain (Fetal) | 27.5 |
| Brain (Hippocampus) | 3.6 |
| Cerebral Cortex pool | 3.3 |
| Brain (Substantia nigra) | 3.0 |
| Brain (Thalamus) | 3.6 |
| Brain (Whole) | 22.5 |
| Spinal Cord | 0.8 |
| Adrenal Gland | 0.7 |
| Pituitary Gland | 4.3 |
| Salivary Gland | 0.1 |
| Thyroid | 11.9 |
| Pancreatic ca. PANC-1 | 3.1 |
| Pancreas pool | 0.2 |

Column A - Rel. Exp. (%) Ag7103, Run 318037113

General_screening₁₃ panel_v1.7 Summary: Ag7103 Highest expression was seen in a brain cancer cell line (CT=21), with high levels of expression seen in all the cancer cell line samples on this panel. This gene or gene product is involved in cell growth and/or proliferation. Targeting this gene or gene product is useful in the treatment of cancers, including breast, brain, ovarian, colon, renal, brain, prostate and melanoma cancers.

AG. CG5577601: Mechanical Stress Induced Protein

Expression of gene CG55776-01 was assessed using the primer-probe sets Ag1489 and Ag4335, described in Tables AGA and AGB. Results of the RTQ-PCR runs are shown in Tables AGC, AGD, AGE, AGF, AGG and AGH.

TABLE AGA
Probe Name Ag1489

| | | | Start | SEQ ID |
|---|---|---|---|---|
| Primers | Sequences | Length | Position | No |
| Forward | 5′-cacagcattgaattgctctgt-3′ | 21 | 7049 | 1417 |
| Probe | TET-5′-tctggattttaccaaatggcacacga-3′- | 26 | 7096 | 1418 |
| | TAMRA | | | |
| Reverse | 5′-gataactttgtggtccattgga-3′ | 22 | 7125 | 1419 |

TABLE AGB
Probe Name Ag4335

| | | | Start | SEQ ID |
|---|---|---|---|---|
| Primers | Sequences | Length | Position | No |
| Forward | 5′-cacctgaagaacccacagatt-3′ | 21 | 2593 | 1420 |
| Probe | TET-5′-tgctattaaaactacagccatgtcaaaga-3′- | 29 | 2627 | 1421 |
| | TAMRA | | | |
| Reverse | 5′-gcttgacatggttgggtttat-3′ | 21 | 2658 | 1422 |

TABLE AGC
AI_comprehensive panel_v1.0

| Tissue Name | A |
|---|---|
| 110967 COPD-F | 1.8 |
| 110980 COPD-F | 12.1 |
| 110968 COPD-M | 2.7 |
| 110977 COPD-M | 0.0 |
| 110989 Emphysema-F | 4.9 |
| 110992 Emphysema-F | 5.7 |
| 110993 Emphysema-F | 3.3 |
| 110994 Emphysema-F | 1.7 |
| 110995 Emphysema-F | 5.2 |
| 110996 Emphysema-F | 3.1 |
| 110997 Asthma-M | 0.5 |
| 111001 Asthma-F | 2.2 |
| 111002 Asthma-F | 2.3 |
| 111003 Atopic Asthma-F | 2.3 |
| 111004 Atopic Asthma-F | 6.1 |
| 111005 Atopic Asthma-F | 2.6 |
| 111006 Atopic Asthma-F | 0.4 |
| 111417 Allergy-M | 3.4 |
| 112347 Allergy-M | 0.1 |
| 112349 Normal Lung-F | 0.2 |
| 112357 Normal Lung-F | 100.0 |
| 112354 Normal Lung-M | 59.0 |
| 112374 Crohns-F | 7.3 |
| 112389 Match Control Crohns-F | 0.5 |
| 112375 Crohns-F | 4.4 |
| 112732 Match Control Crohns-F | 6.1 |
| 112725 Crohns-M | 0.9 |
| 112387 Match Control Crohns-M | 2.5 |
| 112378 Crohns-M | 0.4 |
| 112390 Match Control Crohns-M | 7.5 |
| 112726 Crohns-M | 2.3 |
| 112731 Match Control Crohns-M | 4.7 |
| 112380 Ulcer Col-F | 2.4 |
| 112734 Match Control Ulcer Col-F | 24.1 |
| 112384 Ulcer Col-F | 4.6 |
| 112737 Match Control Ulcer Col-F | 1.6 |
| 112386 Ulcer Col-F | 1.7 |
| 112738 Match Control Ulcer Col-F | 3.2 |
| 112381 Ulcer Col-M | 0.8 |
| 112735 Match Control Ulcer Col-M | 3.8 |
| 112382 Ulcer Col-M | 1.0 |
| 112394 Match Control Ulcer Col-M | 1.9 |
| 112383 Ulcer Col-M | 4.0 |
| 112736 Match Control Ulcer Col-M | 0.6 |
| 112423 Psoriasis-F | 8.9 |
| 112427 Match Control Psoriasis-F | 28.5 |
| 112418 Psoriasis-M | 2.1 |
| 112723 Match Control Psoriasis-M | 6.9 |
| 112419 Psoriasis-M | 4.2 |
| 112424 Match Control Psoriasis-M | 4.4 |
| 112420 Psoriasis-M | 5.4 |
| 112425 Match Control Psoriasis-M | 33.2 |
| 104689 (MF) OA Bone-Backus | 9.0 |
| 104690 (MF) Adj “Normal” Bone-Backus | 8.1 |
| 104691 (MF) OA Synovium-Backus | 54.0 |
| 104692 (BA) OA Cartilage-Backus | 0.3 |
| 104694 (BA) OA Bone-Backus | 20.3 |
| 104695 (BA) Adj “Normal” Bone-Backus | 3.1 |
| 104696 (BA) OA Synovium-Backus | 26.4 |
| 104700 (SS) OA Bone-Backus | 2.8 |
| 104701 (SS) Adj “Normal” Bone-Backus | 8.6 |
| 104702 (SS) OA Synovium-Backus | 14.7 |
| 117093 OA Cartilage Rep7 | 2.1 |
| 112672 OA Bone5 | 11.9 |
| 112673 OA Synovium5 | 5.8 |
| 112674 OA Synovial Fluid cells5 | 6.4 |
| 117100 OA Cartilage Rep14 | 0.7 |
| 112756 OA Bone9 | 2.8 |
| 112757 OA Synovium9 | 0.8 |
| 112758 OA Synovial Fluid Cells9 | 6.1 |
| 117125 RA Cartilage Rep2 | 3.4 |
| 113492 Bone2 RA | 40.3 |
| 113493 Synovium2 RA | 15.8 |
| 113494 Syn Fluid Cells RA | 25.2 |
| 113499 Cartilage4 RA | 33.9 |
| 113500 Bone4 RA | 42.6 |
| 113501 Synovium4 RA | 26.6 |
| 113502 Syn Fluid Cells4 RA | 20.0 |
| 113495 Cartilage3 RA | 27.2 |
| 113496 Bone3 RA | 32.8 |
| 113497 Synovium3 RA | 19.3 |
| 113498 Syn Fluid Cells3 RA | 39.5 |
| 117106 Normal Cartilage Rep20 | 0.0 |
| 113663 Bone3 Normal | 0.7 |
| 113664 Synovium3 Normal | 0.1 |
| 113665 Syn Fluid Cells3 Normal | 0.3 |
| 117107 Normal Cartilage Rep22 | 2.2 |
| 113667 Bone4 Normal | 4.1 |
| 113668 Synovium4 Normal | 4.8 |
| 113669 Syn Fluid Cells4 Normal | 5.8 |

Column A - Rel. Exp. (%) Ag1489, Run 226203351

TABLE AGD
Ardais Panel 1.1

| Tissue Name | A |
|---|---|
| Lung adenocarcinoma SI A | 2.0 |
| Lung adenocarcinoma SI B | 4.2 |
| Lung adenocarcinoma SI B NAT | 78.5 |
| Lung adenocarcinoma SI C | 0.1 |
| Lung adenocarcinoma SI C NAT | 52.1 |
| Lung adenocarcinoma SII A | 1.6 |
| Lung adenocarcinoma SII A NAT | 69.7 |
| Lung adenocarcinoma SII C NAT | 100.0 |
| Lung adenocarcinoma SIII A | 2.3 |
| Lung adenocarcinoma SIII B | 1.6 |
| Lung adenocarcinoma SIII C | 0.0 |
| Lung SCC SI A | 1.3 |
| Lung SCC SI B NAT | 61.6 |
| Lung SCC SI C | 14.5 |
| Lung SCC SI C NAT | 23.0 |
| Lung SCC SI D | 11.8 |
| Lung SCC SI D NAT | 16.6 |
| Lung SCC SII A | 79.6 |
| Lung SCC SII B | 0.7 |
| Lung SCC SIII A | 0.9 |
| Lung SCC SIII A NAT | 18.2 |

Column A - Rel. Exp. (%) Ag1489, Run 312115281

TABLE AGE
General_screening_panel_v1.4

| Tissue Name | A |
|---|---|
| Adipose | 4.7 |
| Melanoma* Hs688(A).T | 0.2 |
| Melanoma* Hs688(B).T | 0.4 |
| Melanoma* M14 | 0.1 |
| Melanoma* LOXIMVI | 0.1 |
| Melanoma* SK-MEL-5 | 0.2 |
| Squamous cell carcinoma SCC-4 | 0.3 |
| Testis Pool | 1.5 |
| Prostate ca.* (bone met) PC-3 | 0.3 |
| Prostate Pool | 0.2 |
| Placenta | 0.1 |
| Uterus Pool | 1.5 |
| Ovarian ca. OVCAR-3 | 1.1 |
| Ovarian ca. SK-OV-3 | 0.0 |
| Ovarian ca. OVCAR-4 | 0.0 |
| Ovarian ca. OVCAR-5 | 0.2 |
| Ovarian ca. IGROV-1 | 0.0 |
| Ovarian ca. OVCAR-8 | 0.1 |
| Ovary | 3.8 |
| Breast ca. MCF-7 | 0.7 |
| Breast ca. MDA-MB-231 | 0.1 |
| Breast ca. BT 549 | 0.2 |
| Breast ca. T47D | 0.3 |
| Breast ca. MDA-N | 0.0 |
| Breast Pool | 2.1 |
| Trachea | 7.0 |
| Lung | 8.5 |
| Fetal Lung | 100.0 |
| Lung ca. NCI-N417 | 0.0 |
| Lung ca. LX-1 | 0.0 |
| Lung ca. NCI-H146 | 10.7 |
| Lung ca. SHP-77 | 0.1 |
| Lung ca. A549 | 3.3 |
| Lung ca. NCI-H526 | 0.2 |
| Lung ca. NCI-H23 | 3.3 |
| Lung ca. NCI-H460 | 0.1 |
| Lung ca. HOP-62 | 2.6 |
| Lung ca. NCI-H522 | 0.0 |
| Liver | 0.1 |
| Fetal Liver | 0.6 |
| Liver ca. HepG2 | 0.0 |
| Kidney Pool | 8.0 |
| Fetal Kidney | 1.6 |
| Renal ca. 786-0 | 0.3 |
| Renal ca. A498 | 0.2 |
| Renal ca. ACHN | 0.0 |
| Renal ca. UO-31 | 0.0 |
| Renal ca. TK-10 | 0.0 |
| Bladder | 0.5 |
| Gastric ca. (liver met.) NCI-N87 | 0.2 |
| Gastric ca. KATO III | 0.0 |
| Colon ca. SW-948 | 0.0 |
| Colon ca. SW480 | 0.1 |
| Colon ca.* (SW480 met) SW620 | 0.0 |
| Colon ca. HT29 | 1.8 |
| Colon ca. HCT-116 | 0.1 |
| Colon ca. CaCo-2 | 3.8 |
| Colon cancer tissue | 0.3 |
| Colon ca. SW1116 | 0.0 |
| Colon ca. Colo-205 | 0.0 |
| Colon ca. SW-48 | 0.0 |
| Colon Pool | 2.0 |
| Small Intestine Pool | 4.2 |
| Stomach Pool | 1.5 |
| Bone Marrow Pool | 1.1 |
| Fetal Heart | 4.9 |
| Heart Pool | 2.7 |
| Lymph Node Pool | 2.2 |
| Fetal Skeletal Muscle | 8.2 |
| Skeletal Muscle Pool | 0.3 |
| Spleen Pool | 2.2 |
| Thymus Pool | 2.1 |
| CNS cancer (glio/astro) U87-MG | 0.3 |
| CNS cancer (glio/astro) U-118-MG | 0.4 |
| CNS cancer (neuro; met) SK-N-AS | 0.0 |
| CNS cancer (astro) SF-539 | 3.9 |
| CNS cancer (astro) SNB-75 | 10.0 |
| CNS cancer (glio) SNB-19 | 0.0 |
| CNS cancer (glio) SF-295 | 4.0 |
| Brain (Amygdala) Pool | 0.5 |
| Brain (cerebellum) | 0.3 |
| Brain (fetal) | 0.4 |
| Brain (Hippocampus) Pool | 1.6 |
| Cerebral Cortex Pool | 1.1 |
| Brain (Substantia nigra) Pool | 0.5 |
| Brain (Thalamus) Pool | 1.4 |
| Brain (whole) | 0.6 |
| Spinal Cord Pool | 0.7 |
| Adrenal Gland | 3.8 |
| Pituitary gland Pool | 3.0 |
| Salivary Gland | 1.7 |
| Thyroid (female) | 1.0 |
| Pancreatic ca. CAPAN2 | 0.0 |
| Pancreas Pool | 2.4 |

Column A - Rel. Exp. (%) Ag4335, Run 222550589

TABLE AGF
Panel 2D

| Tissue Name | A | B |
|---|---|---|
| Normal Colon | 24.0 | 17.2 |
| CC Well to Mod Diff (ODO3866) | 1.4 | 0.4 |
| CC Margin (ODO3866) | 4.6 | 3.6 |
| CC Gr.2 rectosigmoid (ODO3868) | 0.4 | 0.7 |
| CC Margin (ODO3868) | 3.4 | 1.1 |
| CC Mod Diff (ODO3920) | 0.0 | 0.1 |
| CC Margin (ODO3920) | 5.0 | 4.7 |
| CC Gr.2 ascend colon (ODO3921) | 1.0 | 0.5 |
| CC Margin (ODO3921) | 4.1 | 5.0 |
| CC from Partial Hepatectomy | 2.2 | 0.9 |
| (ODO4309) Mets | | |
| Liver Margin (ODO4309) | 3.2 | 2.0 |
| Colon mets to lung (OD04451-01) | 0.4 | 0.7 |
| Lung Margin (OD04451-02) | 6.0 | 5.2 |
| Normal Prostate 6546-1 | 1.1 | 0.9 |
| Prostate Cancer (OD04410) | 1.4 | 0.6 |
| Prostate Margin (OD04410) | 0.2 | 0.7 |
| Prostate Cancer (OD04720-01) | 0.9 | 0.6 |
| Prostate Margin (OD04720-02) | 1.0 | 1.0 |
| Normal Lung 061010 | 100.0 | 100.0 |
| Lung Met to Muscle (ODO4286) | 0.8 | 1.0 |
| Muscle Margin (ODO4286) | 2.0 | 1.3 |
| Lung Malignant Cancer (OD03126) | 11.1 | 8.4 |
| Lung Margin (OD03126) | 64.2 | 51.8 |
| Lung Cancer (OD04404) | 6.6 | 5.1 |
| Lung Margin (OD04404) | 37.4 | 31.9 |
| Lung Cancer (OD04565) | 1.8 | 1.3 |
| Lung Margin (OD04565) | 19.3 | 20.6 |
| Lung Cancer (OD04237-01) | 2.0 | 2.2 |
| Lung Margin (OD04237-02) | 42.3 | 37.6 |
| Ocular Mel Met to Liver (ODO4310) | 0.0 | 0.2 |
| Liver Margin (ODO4310) | 2.3 | 0.5 |
| Melanoma Mets to Lung (OD04321) | 1.9 | 1.8 |
| Lung Margin (OD04321) | 49.0 | 37.9 |
| Normal Kidney | 5.4 | 4.8 |
| Kidney Ca, Nuclear grade 2 (OD04338) | 0.4 | 0.6 |
| Kidney Margin (OD04338) | 3.2 | 2.1 |
| Kidney Ca Nuclear grade 1/2 (OD04339) | 0.7 | 0.3 |
| Kidney Margin (OD04339) | 1.2 | 0.2 |
| Kidney Ca, Clear cell type (OD04340) | 0.4 | 0.4 |
| Kidney Margin (OD04340) | 5.1 | 4.2 |
| Kidney Ca, Nuclear grade 3 (OD04348) | 0.3 | 0.1 |
| Kidney Margin (OD04348) | 2.4 | 1.6 |
| Kidney Cancer (OD04622-01) | 0.1 | 0.2 |
| Kidney Margin (OD04622-03) | 0.0 | 0.1 |
| Kidney Cancer (OD04450-01) | 0.0 | 0.0 |
| Kidney Margin (OD04450-03) | 1.9 | 1.1 |
| Kidney Cancer 8120607 | 0.0 | 0.0 |
| Kidney Margin 8120608 | 0.1 | 0.0 |
| Kidney Cancer 8120613 | 0.0 | 0.0 |
| Kidney Margin 8120614 | 0.1 | 0.1 |
| Kidney Cancer 9010320 | 1.1 | 1.0 |
| Kidney Margin 9010321 | 0.1 | 0.2 |
| Normal Uterus | 3.1 | 2.3 |
| Uterus Cancer 064011 | 24.5 | 18.8 |
| Normal Thyroid | 10.4 | 9.0 |
| Thyroid Cancer 064010 | 0.3 | 0.0 |
| Thyroid Cancer A302152 | 0.5 | 0.5 |
| Thyroid Margin A302153 | 14.5 | 9.2 |
| Normal Breast | 32.1 | 31.9 |
| Breast Cancer (OD04566) | 0.5 | 0.4 |
| Breast Cancer (OD04590-01) | 9.3 | 4.4 |
| Breast Cancer Mets (OD04590-03) | 9.7 | 5.9 |
| Breast Cancer Metastasis (OD04655-05) | 8.2 | 5.8 |
| Breast Cancer 064006 | 8.4 | 5.3 |
| Breast Cancer 1024 | 9.6 | 9.2 |
| Breast Cancer 9100266 | 1.7 | 2.2 |
| Breast Margin 9100265 | 10.7 | 9.6 |
| Breast Cancer A209073 | 11.0 | 10.2 |
| Breast Margin A209073 | 13.6 | 12.5 |
| Normal Liver | 2.2 | 1.9 |
| Liver Cancer 064003 | 0.6 | 0.4 |
| Liver Cancer 1025 | 0.6 | 0.2 |
| Liver Cancer 1026 | 0.0 | 0.1 |
| Liver Cancer 6004-T | 0.9 | 0.3 |
| Liver Tissue 6004-N | 0.2 | 0.2 |
| Liver Cancer 6005-T | 0.0 | 0.2 |
| Liver Tissue 6005-N | 0.0 | 0.0 |
| Normal Bladder | 2.4 | 0.5 |
| Bladder Cancer 1023 | 0.5 | 0.9 |
| Bladder Cancer A302173 | 33.4 | 31.4 |
| Bladder Cancer (OD04718-01) | 0.4 | 0.0 |
| Bladder Normal Adjacent (OD04718-03) | 17.4 | 17.6 |
| Normal Ovary | 22.4 | 14.9 |
| Ovarian Cancer 064008 | 7.1 | 4.4 |
| Ovarian Cancer (OD04768-07) | 2.1 | 1.8 |
| Ovary Margin (OD04768-08) | 1.2 | 1.8 |
| Normal Stomach | 6.4 | 4.4 |
| Gastric Cancer 9060358 | 3.1 | 1.4 |
| Stomach Margin 9060359 | 1.3 | 2.3 |
| Gastric Cancer 9060395 | 2.4 | 4.5 |
| Stomach Margin 9060394 | 5.3 | 3.4 |
| Gastric Cancer 9060397 | 0.3 | 0.7 |
| Stomach Margin 9060396 | 0.7 | 0.1 |
| Gastric Cancer 064005 | 6.8 | 4.4 |

Column A - Rel. Exp. (%) Ag1489, Run 152799815
Column B - Rel. Exp. (%) Ag1489, Run 152799848

TABLE AGG
Panel 4.1D

| Tissue Name | A |
|---|---|
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.6 |
| CD45RO CD4 lymphocyte act | 0.0 |
| CD8 lymphocyte act | 0.0 |
| Secondary CD8 lymphocyte rest | 1.2 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 1.5 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.9 |
| LAK cells PMA/ionomycin | 0.0 |
| NK Cells IL-2 rest | 0.3 |
| Two Way MLR 3 day | 1.7 |
| Two Way MLR 5 day | 0.5 |
| Two Way MLR 7 day | 0.0 |
| PBMC rest | 0.0 |
| PBMC PWM | 0.5 |
| PBMC PHA-L | 0.0 |
| Ramos (B cell) none | 0.7 |
| Ramos (B cell) ionomycin | 0.0 |
| B lymphocytes PWM | 0.0 |
| B lymphocytes CD40L and IL-4 | 0.8 |
| EOL-1 dbcAMP | 34.2 |
| EOL-1 dbcAMP PMA/ionomycin | 18.6 |
| Dendritic cells none | 11.9 |
| Dendritic cells LPS | 0.7 |
| Dendritic cells anti-CD40 | 2.7 |
| Monocytes rest | 1.4 |
| Monocytes LPS | 0.0 |
| Macrophages rest | 2.8 |
| Macrophages LPS | 1.1 |
| HUVEC none | 0.0 |
| HUVEC starved | 0.0 |
| HUVEC IL-1beta | 0.0 |
| HUVEC IFN gamma | 0.9 |
| HUVEC TNF alpha + IFN gamma | 0.0 |
| HUVEC TNF alpha + IL4 | 0.5 |
| HUVEC IL-11 | 0.0 |
| Lung Microvascular EC none | 1.1 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.6 |
| Small airway epithelium TNFalpha + IL-1beta | 0.7 |
| Coronery artery SMC rest | 1.9 |
| Coronery artery SMC TNFalpha + IL-1beta | 1.6 |
| Astrocytes rest | 0.0 |
| Astrocytes TNFalpha + IL-1beta | 0.0 |
| KU-812 (Basophil) rest | 48.6 |
| KU-812 (Basophil) PMA/ionomycin | 26.1 |
| CCD1106 (Keratinocytes) none | 0.0 |
| CCD1106 (Keratinocytes) TNFalpha + IL-1beta | 0.0 |
| Liver cirrhosis | 6.0 |
| NCI-H292 none | 0.7 |
| NCI-H292 IL-4 | 0.0 |
| NCI-H292 IL-9 | 0.7 |
| NCI-H292 IL-13 | 0.4 |
| NCI-H292 IFN gamma | 1.7 |
| HPAEC none | 0.0 |
| HPAEC TNF alpha + IL-1 beta | 0.0 |
| Lung fibroblast none | 26.8 |
| Lung fibroblast TNF alpha + IL-1 beta | 2.0 |
| Lung fibroblast IL-4 | 20.3 |
| Lung fibroblast IL-9 | 26.2 |
| Lung fibroblast IL-13 | 37.4 |
| Lung fibroblast IFN gamma | 44.1 |
| Dermal fibroblast CCD1070 rest | 3.0 |
| Dermal fibroblast CCD1070 TNF alpha | 0.7 |
| Dermal fibroblast CCD1070 IL-1 beta | 2.2 |
| Dermal fibroblast IFN gamma | 12.8 |
| Dermal fibroblast IL-4 | 48.3 |
| Dermal Fibroblasts rest | 17.9 |
| Neutrophils TNFa + LPS | 0.0 |
| Neutrophils rest | 0.7 |
| Colon | 10.7 |
| Lung | 100.0 |
| Thymus | 27.9 |
| Kidney | 10.0 |

Column A - Rel. Exp. (%) Ag4335, Run 184793354

TABLE AGH
Panel 5D

| Tissue Name | A |
|---|---|
| 97457_Patient-02go_adipose | 100.0 |
| 97476_Patient-07sk_skeletal muscle | 23.3 |
| 97477_Patient-07ut_uterus | 8.7 |
| 97478_Patient-07pl_placenta | 7.9 |
| 97481_Patient-08sk_skeletal muscle | 54.7 |
| 97482_Patient-08ut_uterus | 1.4 |
| 97483_Patient-08pl_placenta | 12.1 |
| 97486_Patient-09sk_skeletal muscle | 3.3 |
| 97487_Patient-09ut_uterus | 28.7 |
| 97488_Patient-09pl_placenta | 2.6 |
| 97492_Patient-10ut_uterus | 8.4 |
| 97493_Patient-10pl_placenta | 9.5 |
| 97495_Patient-11go_adipose | 39.5 |
| 97496_Patient-11sk_skeletal muscle | 4.4 |
| 97497_Patient-11ut_uterus | 11.1 |
| 97498_Patient-11pl_placenta | 4.9 |
| 97500_Patient-12go_adipose | 24.0 |
| 97501_Patient-12sk_skeletal muscle | 6.0 |
| 97502_Patient-12ut_uterus | 2.6 |
| 97503_Patient-12pl_placenta | 2.9 |
| 94721_Donor 2 U - A_Mesenchymal Stem Cells | 2.0 |
| 94722_Donor 2 U - B_Mesenchymal Stem Cells | 0.7 |
| 94723_Donor 2 U - C_Mesenchymal Stem Cells | 1.7 |
| 94709_Donor 2 AM - A_adipose | 2.0 |
| 94710_Donor 2 AM - B_adipose | 2.1 |
| 94711_Donor 2 AM - C_adipose | 0.3 |
| 94712_Donor 2 AD - A_adipose | 2.8 |
| 94713_Donor 2 AD - B_adipose | 3.4 |
| 94714_Donor 2 AD - C_adipose | 4.5 |
| 94742_Donor 3 U - A_Mesenchymal Stem Cells | 0.2 |
| 94743_Donor 3 U - B_Mesenchymal Stem Cells | 1.1 |
| 94730_Donor 3 AM - A_adipose | 4.2 |
| 94731_Donor 3 AM - B_adipose | 2.0 |
| 94732_Donor 3 AM - C_adipose | 1.8 |
| 94733_Donor 3 AD - A_adipose | 0.5 |
| 94734_Donor 3 AD - B_adipose | 0.3 |
| 94735_Donor 3 AD - C_adipose | 0.2 |
| 77138_Liver_HepG2untreated | 0.5 |
| 73556_Heart_Cardiac stromal cells (primary) | 0.0 |
| 81735_Small Intestine | 49.3 |
| 72409_Kidney_Proximal Convoluted Tubule | 0.6 |
| 82685_Small intestine_Duodenum | 5.2 |
| 90650_Adrenal_Adrenocortical adenoma | 5.3 |
| 72410_Kidney_HRCE | 0.0 |
| 72411_Kidney_HRE | 0.8 |
| 73139_Uterus_Uterine smooth muscle cells | 4.5 |

Column A - Rel. Exp. (%) Ag4335, Run 258657707

AI_comprehensive panel₁₃ v1.0 Summary: Ag1489 Expression of this transcript was induced in most rheumatoid arthritis (RA) tissues and in synovium and bone tissues from osteoarthritis (OA) patients. This gene was also highly and consistently expressed in normal lung in 2 out of 3 normal lungs tissues on this panel and in panels 1.4 and 4.1. The expression in diseased lung was reduced or absent. These data show that lung expression of the protein encoded for by this transcript serves an important function that is lost in disease states, but that OA/RA promotes the expression of this gene in the joint. Therapies designed with the protein encoded by this transcript are important for the treatment of OA/RA and lung diseases such as chronic pulmonary obstructive disease, emphysema, allergy and asthma.

Ardais Panel 1.1 Summary: Ag1489 Highest expression is detected in normal adjacent lung tissue sample (CT=25). This gene is expressed at higher levels in normal tissue relative to the cancer samples. This expression profile is in agreement with results in panel 2D. This gene codes for a protein containing ig and leucine rich repeat regions and is similar to adlican. Members of this family are integral basement membrane proteins with a variety of functions, including matrix assembly and organization, regulation of cell growth, adhesion and migration. The LRR domain in particular is thought to play an important role in these processes (Hocking A M, Shinomura T, McQuillan D J., 1998, Leucine-rich repeat glycoproteins of the extracellular matrix. Matrix Biol. 17(1):1-19). The expression profile of this gene indicates an important role in lung biology and therefore modulation of this gene, expressed protein and/or use of agonists will be useful in the treatment of lung cancer.

General_screening₁₃ panel_v1.4 Summary: Ag4335 Highest expression of this gene was seen in fetal lung (CT=25.4), with overall expression associated with normal tissues. Expression of this gene was higher in fetal lung relative to adult lung (CT=29) and in fetal skeletal muscle (CT=29) relative to adult skeletal muscle (CT=33). This gene or expressed protein is involved in the development and homeostasis of these organs. Therapeutic modulation of this gene and/or expressed protein is useful in the treatment of lung and skeletal muscle related diseases.

This gene was also expressed at significant levels in a variety of metabolic tissues including adipose, adult and fetal liver, adult and fetal heart, pituitary, thyroid and pancreas. Modulation of this gene and/or expressed protein is useful in the treatment of metabolic disorders, including obesity and diabetes.

Moderate to low expression of this gene was also seen in all the regions of brain examined including amygdala, hippocampus, substantia nigra, thalamus, cerebellum, cerebral cortex, and spinal cord. Therapeutic modulation of this gene or gene product is useful in the treatment of central nervous system disorders such as Alzheimer's disease, Parkinson's disease, epilepsy, multiple sclerosis, schizophrenia and depression.

Panel 2D Summary: Ag1489 Highest expression of this gene was detected in normal lung (CTs=27.8). This gene showed higher expression in all the normal adjacent lung, kidney and colon tissues relative to cancer samples. Modulation of this gene, expressed protein and/or use of antibodies or agonists targeting this gene or its protein product are useful in the treatment of these cancers.

Panel 4.1D Summary: Ag4335 Highest expression of this gene was detected in normal lung (CT=31). This gene was also expressed in lung fibroblasts, EOL-1 cells and basophils. Expression of the gene was downregulated by TNF-alpha and IL1-beta in lung fibroblasts. Based on the expression profile in this panel as well as that in the A/I panel, therapeutics designed with the encoded protein are useful in the treatment of chronic obstructive pulmonary disease, emphysema, allergy and asthma.

Panel 5D Summary: Ag4335 Highest expression of this gene was detected in adipose tissue from a diabetic patient (CT=30.8). Significant expression of this gene was also seen in skeletal muscle, adipose, uterus, placenta and small intestine. Modulation of this gene or its expressed protein is useful in the treatment of metabolic diseases such as obesity and diabetes.

AH. CG5578401 and CG5578403: Nephrin-like

Expression of gene CG55784-03 and variant CG55784-01 was assessed using the primer-probe sets Ag2844 and Ag6457, described in Tables AHA and AHB. Results of the RTQ-PCR runs are shown in Tables AHC, AHD and AHE. Please note that the probe and primer set Ag6457 is specific to the CG55784-03 only.

TABLE AHA
Probe Name Ag2844

| | | | Start | SEQ ID |
|---|---|---|---|---|
| Primers | Sequences | Length | Position | No |
| Forward | 5′-ctttccactgctctgcaaag-3′ | 20 | 1053 | 1423 |
| Probe | TET-5′-aacccagctgtcacccagtacaggtg-3′- | 26 | 1076 | 1424 |
| | TAMRA | | | |
| Reverse | 5′-gtcctgtacacctctccagatg-3′ | 22 | 1131 | 1425 |

TABLE AHB
Probe Name Ag6457

| | | | Start | SEQ ID |
|---|---|---|---|---|
| Primers | Sequences | Length | Position | No |
| Forward | 5′-cgacttccagaccatctacaact-3′ | 23 | 1723 | 1426 |
| Probe | TET-5′-cttcggctccgacactgagatcatc-3′- | 25 | 1762 | 1427 |
| | TAMRA | | | |
| Reverse | 5′-ggcacagactcttgctcctt-3′ | 20 | 1793 | 1428 |

TABLE AHC
General_screening_panel_v1.6

| Tissue Name | A |
|---|---|
| Adipose | 0.0 |
| Melanoma* Hs688(A).T | 13.6 |
| Melanoma* Hs688(B).T | 15.9 |
| Melanoma* M14 | 0.2 |
| Melanoma* LOXIMVI | 0.0 |
| Melanoma* SK-MEL-5 | 0.0 |
| Squamous cell carcinoma SCC-4 | 0.3 |
| Testis Pool | 0.0 |
| Prostate ca.* (bone met) PC-3 | 0.0 |
| Prostate Pool | 0.8 |
| Placenta | 0.4 |
| Uterus Pool | 0.4 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 1.7 |
| Ovarian ca. IGROV-1 | 1.7 |
| Ovarian ca. OVCAR-8 | 1.8 |
| Ovary | 0.7 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 0.0 |
| Trachea | 2.0 |
| Lung | 0.4 |
| Fetal Lung | 5.4 |
| Lung ca. NCI-N417 | 0.0 |
| Lung ca. LX-1 | 0.0 |
| Lung ca. NCI-H146 | 3.7 |
| Lung ca. SHP-77 | 0.0 |
| Lung ca. A549 | 9.0 |
| Lung ca. NCI-H526 | 1.1 |
| Lung ca. NCI-H23 | 0.0 |
| Lung ca. NCI-H460 | 0.1 |
| Lung ca. HOP-62 | 0.5 |
| Lung ca. NCI-H522 | 0.0 |
| Liver | 0.0 |
| Fetal Liver | 0.0 |
| Liver ca. HepG2 | 0.0 |
| Kidney Pool | 0.0 |
| Fetal Kidney | 0.1 |
| Renal ca. 786-0 | 0.0 |
| Renal ca. A498 | 3.2 |
| Renal ca. ACHN | 35.8 |
| Renal ca. UO-31 | 0.0 |
| Renal ca. TK-10 | 0.0 |
| Bladder | 0.4 |
| Gastric ca. (liver met.) NCI-N87 | 0.0 |
| Gastric ca. KATO III | 2.4 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 0.0 |
| Colon ca. SW1116 | 0.0 |
| Colon ca. Colo-205 | 0.0 |
| Colon ca. SW-48 | 0.0 |
| Colon Pool | 0.4 |
| Small Intestine Pool | 1.4 |
| Stomach Pool | 0.0 |
| Bone Marrow Pool | 0.3 |
| Fetal Heart | 1.5 |
| Heart Pool | 0.5 |
| Lymph Node Pool | 1.0 |
| Fetal Skeletal Muscle | 0.0 |
| Skeletal Muscle Pool | 0.0 |
| Spleen Pool | 1.1 |
| Thymus Pool | 0.4 |
| CNS cancer (glio/astro) U87-MG | 0.0 |
| CNS cancer (glio/astro) U-118-MG | 8.0 |
| CNS cancer (neuro; met) SK-N-AS | 2.9 |
| CNS cancer (astro) SF-539 | 0.0 |
| CNS cancer (astro) SNB-75 | 0.8 |
| CNS cancer (glio) SNB-19 | 4.0 |
| CNS cancer (glio) SF-295 | 0.7 |
| Brain (Amygdala) Pool | 32.3 |
| Brain (cerebellum) | 100.0 |
| Brain (fetal) | 41.2 |
| Brain (Hippocampus) Pool | 29.3 |
| Cerebral Cortex Pool | 35.8 |
| Brain (Substantia nigra) Pool | 25.5 |
| Brain (Thalamus) Pool | 56.3 |
| Brain (whole) | 79.0 |
| Spinal Cord Pool | 16.8 |
| Adrenal Gland | 2.0 |
| Pituitary gland Pool | 0.0 |
| Salivary Gland | 1.6 |
| Thyroid (female) | 0.0 |
| Pancreatic ca. CAPAN2 | 0.0 |
| Pancreas Pool | 0.4 |

Column A - Rel. Exp. (%) Ag6457, Run 277250272

TABLE AHD
Panel 4.1D

| Tissue Name | A |
|---|---|
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 74.7 |
| CD45RO CD4 lymphocyte act | 0.3 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 2.4 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 4.4 |
| Coronery artery SMC TNFalpha + IL-1beta | 14.1 |
| Astrocytes rest | 2.9 |
| Astrocytes TNFalpha + IL-1beta | 5.2 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL-1beta | 0.0 |
| Liver cirrhosis | 0.0 |
| NCI-H292 none | 10.2 |
| NCI-H292 IL-4 | 10.2 |
| NCI-H292 IL-9 | 0.0 |
| NCI-H292 IL-13 | 14.3 |
| NCI-H292 IFN gamma | 0.0 |
| HPAEC none | 0.0 |
| HPAEC TNF alpha + IL-1 beta | 0.0 |
| Lung fibroblast none | 11.5 |
| Lung fibroblast TNF alpha + IL-1 beta | 6.6 |
| Lung fibroblast IL-4 | 20.9 |
| Lung fibroblast IL-9 | 5.1 |
| Lung fibroblast IL-13 | 5.3 |
| Lung fibroblast IFN gamma | 23.2 |
| Dermal fibroblast CCD1070 rest | 100.0 |
| Dermal fibroblast CCD1070 TNF alpha | 59.5 |
| Dermal fibroblast CCD1070 IL-1 beta | 59.0 |
| Dermal fibroblast IFN gamma | 2.9 |
| Dermal fibroblast IL-4 | 9.3 |
| Dermal Fibroblasts rest | 7.5 |
| Neutrophils TNFa + LPS | 0.0 |
| Neutrophils rest | 0.0 |
| Colon | 2.8 |
| Lung | 0.0 |
| Thymus | 0.0 |
| Kidney | 0.0 |

Column A - Rel. Exp.(%) Ag6457, Run 269261799

TABLE AHE
Panel 4D

| Tissue Name | A |
|---|---|
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 43.5 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 1.7 |
| Coronery artery SMC TNFalpha + IL-1beta | 5.1 |
| Astrocytes rest | 2.0 |
| Astrocytes TNFalpha + IL-1beta | 3.7 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL-1beta | 0.0 |
| Liver cirrhosis | 0.3 |
| Lupus kidney | 0.0 |
| NCI-H292 none | 1.8 |
| NCI-H292 IL-4 | 27.2 |
| NCI-H292 IL-9 | 3.7 |
| NCI-H292 IL-13 | 17.8 |
| NCI-H292 IFN gamma | 0.0 |
| HPAEC none | 0.0 |
| HPAEC TNF alpha + IL-1 beta | 0.0 |
| Lung fibroblast none | 4.3 |
| Lung fibroblast TNF alpha + IL-1 beta | 3.4 |
| Lung fibroblast IL-4 | 12.5 |
| Lung fibroblast IL-9 | 2.6 |
| Lung fibroblast IL-13 | 5.3 |
| Lung fibroblast IFN gamma | 5.2 |
| Dermal fibroblast CCD1070 rest | 90.1 |
| Dermal fibroblast CCD1070 TNF alpha | 100.0 |
| Dermal fibroblast CCD1070 IL-1 beta | 39.8 |
| Dermal fibroblast IFN gamma | 1.6 |
| Dermal fibroblast IL-4 | 4.5 |
| IBD Colitis 2 | 0.0 |
| IBD Crohn's | 0.2 |
| Colon | 1.3 |
| Lung | 0.0 |
| Thymus | 0.4 |
| Kidney | 0.5 |

Column A - Rel. Exp.(%) Ag2844, Run 164299480

General_screening₁₃ panel_v1.6 Summary: Ag6457 This probe/primer set was specific for the CG55784-03 gene variant. Expression of this gene was highest in cerebellum (CT=29.5). Highly brain-preferential expression of the CG55784-03 gene demonstrates a specific role for this gene product in the brain. This gene encodes a protein that is homologous to a neural cell adhesion molecule (NCAM). NCAM related proteins, such as Nr-CAM, play a critical role in neurite extension (Sakurai et al., J Cell Biol 2001 154:1259-73). Eintroduction of ligands specific for this gene product, such as contactin, in directed brain regions have utility in fostering focal neurite outgrowth and in therapeutically countering neurite degeneration of neurodegenerative diseases such as Alzheimer's, ataxias, and Parkinson's disease.

In addition, the expression of this gene was relatively high in the normal brain samples compared to the cancer cell lines derived from brain cancer. Expression of this gene is useful as a marker to differentiate between normal and cancerous tissue. There were also significantly higher levels of expression in renal cancer cell lines compared to a normal kidney sample. Expression of this gene is also useful as a marker in renal cancer.

Panel 4.1D Summary: Ag6457 This probe/primer set is specific for the CG55784-03 gene variant. Expression of this gene was highest in dermal fibroblasts (CT=31.8) and was relatively treatment independent. This gene was also expressed in CD45RA (naive) T cells. Therapeutic modulation of this gene or its protein product is useful in the treatment of psoriasis or diseases mediated by activated T cells.

Panel 4D Summary: Ag2844 This transcript was induced in IL-4 and IL-13 treated NCI-H292 cells, expressed constitutively in a dermal fibroblast cell line and was slightly induced by IL-4 in lung fibroblasts. CD45RA (naive) T cells also expressed the transcript. The transcript encodes a NCAM-like molecule. Based on the expression pattern of the transcript, the homology to NCAM protein, and the regulation of transcript expression by IL-4 and IL-13, therapeutics designed with the protein encoded for by this transcript are important in the treatment of asthma and COPD.

AI. CG55790-01 and CG55790-02: B7-H2B

Expression of gene CG55790-02 and variant CG55790-01 was assessed using the primer-probe sets Ag1845, Ag2589, Ag2621, Ag2915, and Ag8156, described in Tables AIA, AIB, AIC, AID, and AIE. Results of the RTQ-PCR runs are shown in Tables AIF, AIG, AIH, AII, All and AIKPlease note that only the probe and primer sets Ag2589, Ag2621, and Ag2915 correspond to the CG55790-01 variant.

TABLE AIA
Probe Name Ag1845

| | | | Start | SEQ ID |
|---|---|---|---|---|
| Primers | Sequences | Length | Position | No |
| Forward | 5′-catcacgtgtgagctcacagt-3′ | 21 | 7434 | 1429 |
| Probe | TET-5′-cttccacatggtgcactgctgct-3′-TAMRA | 23 | 7393 | 1430 |
| Reverse | 5′-agaatttgcagacacagcaatt-3′ | 22 | 7366 | 1431 |

TABLE AIB
Probe Name Ag2589

| | | | Start | SEQ ID |
|---|---|---|---|---|
| Primers | Sequences | Length | Position | No |
| Forward | 5′-gagctcaccttcacgtgtacat-3′ | 22 | 460 | 1432 |
| Probe | TET-5′-ctaccccaggcccaacgtgtactg-3′- | 24 | 492 | 1433 |
| | TAMRA | | | |
| Reverse | 5′-gctgttgtccgtcttattgatc-3′ | 22 | 516 | 1434 |

TABLE AIC
Probe Name Ag2621

| | | | Start | SEQ ID |
|---|---|---|---|---|
| Primers | Sequences | Length | Position | No |
| Forward | 5′-gagctcaccttcacgtgtacat-3′ | 22 | 460 | 1435 |
| Probe | TET-5′-ctaccccaggcccaacgtgtactg-3′- | 24 | 492 | 1436 |
| | TAMRA | | | |
| Reverse | 5′-gctgttgtccgtcttattgatc-3′ | 22 | 516 | 1437 |

TABLE AID
Probe Name Ag2915

| | | | Start | SEQ ID |
|---|---|---|---|---|
| Primers | Sequences | Length | Position | No |
| Forward | 5′-gagctcaccttcacgtgtacat-3′ | 22 | 460 | 1438 |
| Probe | TET-5′-ctaccccaggcccaacgtgtactg-3′- | 24 | 492 | 1439 |
| | TAMRA | | | |
| Reverse | 5′-gctgttgtccgtcttattgatc-3′ | 22 | 516 | 1440 |

TABLE AIE
Probe Name Ag8156

| | | | | |
|---|---|---|---|---|
| | | | Start | SEQ ID |
| Primers | Sequences | Length | Position | No |

| | | | | |
|---|---|---|---|---|
| Forward | 5′-tgccagctgtctttcagagt-3′ | 20 | 955 | 1441 |
| Probe | TET-5′-ctttcaaaaggcaaaagtcccttgcc-3′- | 26 | 984 | 1439 |
| | TAMRA | | | |
| Reverse | 5′-gcgtctccaggaacaacagt-3′ | 20 | 1017 | 1443 |

TABLE AIF
AI_comprehensive panel_v1.0

| Tissue Name | A |
|---|---|
| 110967 COPD-F | 8.5 |
| 110980 COPD-F | 4.8 |
| 110968 COPD-M | 6.7 |
| 110977 COPD-M | 8.5 |
| 110989 Emphysema-F | 12.9 |
| 110992 Emphysema-F | 7.5 |
| 110993 Emphysema-F | 8.5 |
| 110994 Emphysema-F | 3.0 |
| 110995 Emphysema-F | 14.9 |
| 110996 Emphysema-F | 1.6 |
| 110997 Asthma-M | 14.1 |
| 111001 Asthma-F | 7.9 |
| 111002 Asthma-F | 12.6 |
| 111003 Atopic Asthma-F | 13.4 |
| 111004 Atopic Asthma-F | 16.5 |
| 111005 Atopic Asthma-F | 7.7 |
| 111006 Atopic Asthma-F | 0.9 |
| 111417 Allergy-M | 6.3 |
| 112347 Allergy-M | 1.7 |
| 112349 Normal Lung-F | 0.5 |
| 112357 Normal Lung-F | 1.7 |
| 112354 Normal Lung-M | 2.9 |
| 112374 Crohns-F | 3.3 |
| 112389 Match Control Crohns-F | 17.9 |
| 112375 Crohns-F | 1.8 |
| 112732 Match Control Crohns-F | 39.5 |
| 112725 Crohns-M | 1.7 |
| 112387 Match Control Crohns-M | 4.6 |
| 112378 Crohns-M | 0.7 |
| 112390 Match Control Crohns-M | 12.1 |
| 112726 Crohns-M | 10.1 |
| 112731 Match Control Crohns-M | 18.9 |
| 112380 Ulcer Col-F | 9.6 |
| 112734 Match Control Ulcer Col-F | 100.0 |
| 112384 Ulcer Col-F | 17.1 |
| 112737 Match Control Ulcer Col-F | 13.5 |
| 112386 Ulcer Col-F | 1.8 |
| 112738 Match Control Ulcer Col-F | 1.4 |
| 112381 Ulcer Col-M | 7.3 |
| 112735 Match Control Ulcer Col-M | 7.2 |
| 112382 Ulcer Col-M | 12.5 |
| 112394 Match Control Ulcer Col-M | 2.3 |
| 112383 Ulcer Col-M | 6.3 |
| 112736 Match Control Ulcer Col-M | 13.0 |
| 112423 Psoriasis-F | 7.1 |
| 112427 Match Control Psoriasis-F | 14.5 |
| 112418 Psoriasis-M | 6.6 |
| 112723 Match Control Psoriasis-M | 1.0 |
| 112419 Psoriasis-M | 9.9 |
| 112424 Match Control Psoriasis-M | 10.7 |
| 112420 Psoriasis-M | 21.5 |
| 112425 Match Control Psoriasis-M | 13.8 |
| 104689 (MF) OA Bone-Backus | 17.1 |
| 104690 (MF) Adj “Normal” Bone-Backus | 6.8 |
| 104691 (MF) OA Synovium-Backus | 14.6 |
| 104692 (BA) OA Cartilage-Backus | 0.4 |
| 104694 (BA) OA Bone-Backus | 13.3 |
| 104695 (BA) Adj “Normal” Bone-Backus | 9.3 |
| 104696 (BA) OA Synovium-Backus | 7.9 |
| 104700 (SS) OA Bone-Backus | 7.5 |
| 104701 (SS) Adj “Normal” Bone-Backus | 7.7 |
| 104702 (SS) OA Synovium-Backus | 24.5 |
| 117093 OA Cartilage Rep7 | 7.7 |
| 112672 OA Bone5 | 6.2 |
| 112673 OA Synovium5 | 1.1 |
| 112674 OA Synovial Fluid cells5 | 2.6 |
| 117100 OA Cartilage Rep14 | 4.3 |
| 112756 OA Bone9 | 14.6 |
| 112757 OA Synovium9 | 7.1 |
| 112758 OA Synovial Fluid Cells9 | 4.2 |
| 117125 RA Cartilage Rep2 | 4.7 |
| 113492 Bone2 RA | 4.8 |
| 113493 Synovium2 RA | 3.6 |
| 113494 Syn Fluid Cells RA | 7.5 |
| 113499 Cartilage4 RA | 9.6 |
| 113500 Bone4 RA | 10.7 |
| 113501 Synovium4 RA | 6.9 |
| 113502 Syn Fluid Cells4 RA | 5.4 |
| 113495 Cartilage3 RA | 4.0 |
| 113496 Bone3 RA | 7.2 |
| 113497 Synovium3 RA | 4.4 |
| 113498 Syn Fluid Cells3 RA | 6.2 |
| 117106 Normal Cartilage Rep20 | 7.3 |
| 113663 Bone3 Normal | 0.2 |
| 113664 Synovium3 Normal | 0.0 |
| 113665 Syn Fluid Cells3 Normal | 0.4 |
| 117107 Normal Cartilage Rep22 | 3.0 |
| 113667 Bone4 Normal | 4.3 |
| 113668 Synovium4 Normal | 2.7 |
| 113669 Syn Fluid Cells4 Normal | 7.0 |

Column A - Rel. Exp.(%) Ag1845, Run 217699370

TABLE AIG
CNS_neurodegeneration_v1.0

| Tissue Name | A | B | C | D | E | F |
|---|---|---|---|---|---|---|
| AD 1 Hippo | 37.6 | 19.1 | 26.4 | 26.1 | 42.3 | 37.9 |
| AD 2 Hippo | 26.4 | 11.1 | 26.6 | 21.2 | 27.0 | 33.4 |
| AD 3 Hippo | 39.5 | 15.6 | 24.1 | 15.1 | 20.4 | 32.3 |
| AD 4 Hippo | 24.3 | 10.6 | 16.8 | 10.7 | 20.9 | 28.5 |
| AD 5 Hippo | 29.5 | 23.8 | 26.2 | 22.7 | 32.3 | 28.3 |
| AD 6 Hippo | 62.0 | 45.4 | 57.4 | 45.1 | 56.3 | 75.8 |
| Control 2 Hippo | 57.4 | 22.4 | 45.7 | 29.1 | 41.5 | 50.7 |
| Control 4 Hippo | 22.8 | 10.9 | 27.4 | 20.4 | 24.5 | 25.3 |
| Control (Path) 3 Hippo | 22.7 | 100.0 | 14.6 | 12.3 | 14.7 | 12.5 |
| AD 1 Temporal Ctx | 48.0 | 27.2 | 33.9 | 27.9 | 33.7 | 44.8 |
| AD 2 Temporal Ctx | 39.0 | 30.1 | 25.2 | 21.5 | 33.7 | 29.9 |
| AD 3 Temporal Ctx | 42.3 | 19.8 | 19.5 | 14.8 | 13.4 | 16.3 |
| AD 4 Temporal Ctx | 35.1 | 14.7 | 24.3 | 22.7 | 33.7 | 26.2 |
| AD 5 Inf Temporal Ctx | 70.2 | 28.3 | 50.3 | 45.4 | 63.7 | 60.3 |
| AD 5 Sup Temporal Ctx | 48.6 | 17.8 | 37.4 | 32.1 | 45.1 | 35.1 |
| AD 6 Inf Temporal Ctx | 100.0 | 19.6 | 100.0 | 100.0 | 100.0 | 100.0 |
| AD 6 Sup Temporal Ctx | 57.4 | 27.0 | 47.3 | 33.4 | 49.0 | 59.9 |
| Control 1 Temporal Ctx | 11.8 | 9.7 | 14.8 | 8.8 | 14.2 | 14.0 |
| Control 2 Temporal Ctx | 33.7 | 23.0 | 30.8 | 26.8 | 11.3 | 45.4 |
| Control 3 Temporal Ctx | 20.6 | 15.9 | 18.6 | 13.7 | 17.2 | 18.8 |
| Control 3 Temporal Ctx | 21.9 | 4.5 | 14.9 | 12.5 | 26.6 | 23.7 |
| Control (Path) 1 | 27.2 | 26.1 | 27.5 | 19.6 | 28.5 | 31.4 |
| Temporal Ctx | | | | | | |
| Control (Path) 2 | 16.7 | 7.2 | 18.3 | 16.5 | 17.7 | 23.2 |
| Temporal Ctx | | | | | | |
| Control (Path) 3 | 23.2 | 12.4 | 7.9 | 6.0 | 7.2 | 10.8 |
| Temporal Ctx | | | | | | |
| Control (Path) 4 | 17.4 | 35.4 | 15.1 | 12.9 | 17.3 | 19.6 |
| Temporal Ctx | | | | | | |
| AD 1 Occipital Ctx | 30.4 | 9.2 | 22.2 | 14.5 | 25.0 | 23.3 |
| AD 2 Occipital Ctx | 1.3 | 26.1 | 1.9 | 14.8 | 2.5 | 1.5 |
| (Missing) | | | | | | |
| AD 3 Occipital Ctx | 26.8 | 16.0 | 17.3 | 14.6 | 23.0 | 20.9 |
| AD 4 Occipital Ctx | 45.1 | 13.0 | 27.9 | 22.5 | 27.2 | 0.1 |
| AD 5 Occipital Ctx | 33.0 | 57.8 | 27.5 | 31.2 | 27.9 | 32.1 |
| AD 6 Occipital Ctx | 47.3 | 22.7 | 36.6 | 19.3 | 40.6 | 44.1 |
| Control 1 Occipital Ctx | 27.5 | 16.2 | 16.4 | 11.3 | 12.8 | 16.5 |
| Control 2 Occipital Ctx | 39.8 | 15.1 | 28.3 | 29.3 | 42.0 | 42.3 |
| Control 3 Occipital Ctx | 20.7 | 16.0 | 18.7 | 13.0 | 18.6 | 16.4 |
| Control 4 Occipital Ctx | 35.1 | 23.5 | 17.6 | 19.6 | 24.3 | 22.5 |
| Control (Path) 1 | 47.0 | 23.3 | 44.4 | 35.8 | 47.0 | 51.8 |
| Occipital Ctx | | | | | | |
| Control (Path) 2 | 18.3 | 32.5 | 21.9 | 17.6 | 31.4 | 23.8 |
| Occipital Ctx | | | | | | |
| Control (Path) 3 | 23.8 | 8.2 | 12.6 | 15.2 | 12.2 | 22.5 |
| Occipital Ctx | | | | | | |
| Control (Path) 4 | 22.8 | 14.6 | 20.9 | 17.8 | 21.0 | 23.2 |
| Occipital Ctx | | | | | | |
| Control 1 Parietal Ctx | 34.6 | 13.6 | 25.0 | 20.7 | 22.5 | 28.3 |
| Control 2 Parietal Ctx | 48.0 | 18.0 | 36.9 | 25.3 | 35.4 | 40.1 |
| Control 3 Parietal Ctx | 37.6 | 30.6 | 26.4 | 20.3 | 31.4 | 24.1 |
| Control (Path) 1 | 29.5 | 11.9 | 28.7 | 23.3 | 28.7 | 31.9 |
| Parietal Ctx | | | | | | |
| Control (Path) 2 | 24.5 | 21.8 | 19.8 | 17.3 | 29.9 | 28.3 |
| Parietal Ctx | | | | | | |
| Control (Path) 3 | 15.6 | 17.6 | 13.2 | 9.0 | 8.4 | 16.8 |
| Parietal Ctx | | | | | | |
| Control (Path) 4 | 23.8 | 11.7 | 22.7 | 21.0 | 23.0 | 22.5 |
| Parietal Ctx | | | | | | |

Column A - Rel. Exp. (%) Ag1845, Run 207807655
Column B - Rel. Exp. (%) Ag1845, Run 224079124
Column C - Rel. Exp. (%) Ag2589, Run 208776915
Column D - Rel. Exp. (%) Ag2621, Run 208393684
Column E - Rel. Exp. (%) Ag2915, Run 209735956
Column F - Rel. Exp. (%) Ag2915, Run 266906552

TABLE AIH
Panel 1.3D

| Tissue Name | A | B | C | D | E |
|---|---|---|---|---|---|
| Liver adenocarcinoma | 3.9 | 4.7 | 23.8 | 17.6 | 22.8 |
| Pancreas | 0.9 | 0.3 | 0.8 | 1.6 | 2.3 |
| Pancreatic ca. CAPAN 2 | 0.0 | 0.0 | 2.6 | 0.5 | 1.0 |
| Adrenal gland | 1.6 | 2.1 | 3.7 | 3.2 | 1.9 |
| Thyroid | 2.2 | 2.9 | 4.2 | 5.3 | 3.3 |
| Salivary gland | 2.0 | 3.1 | 6.3 | 4.0 | 4.2 |
| Pituitary gland | 0.9 | 1.1 | 2.0 | 2.0 | 1.4 |
| Brain (fetal) | 0.2 | 0.9 | 4.6 | 6.0 | 3.3 |
| Brain (whole) | 31.2 | 31.4 | 90.8 | 90.8 | 77.4 |
| Brain (amygdala) | 28.5 | 24.7 | 34.9 | 31.4 | 34.4 |
| Brain (cerebellum) | 3.0 | 1.5 | 21.6 | 18.6 | 20.7 |
| Brain (hippocampus) | 100.0 | 100.0 | 39.2 | 29.9 | 27.7 |
| Brain (substantia nigra) | 26.1 | 21.3 | 86.5 | 60.7 | 67.8 |
| Brain (thalamus) | 29.9 | 31.0 | 89.5 | 49.7 | 59.0 |
| Cerebral Cortex | 20.0 | 16.4 | 46.3 | 33.2 | 38.4 |
| Spinal cord | 15.0 | 12.7 | 29.9 | 20.2 | 31.0 |
| glio/astro U87-MG | 2.6 | 3.5 | 18.7 | 13.3 | 18.9 |
| glio/astro U-118-MG | 1.3 | 0.3 | 0.6 | 1.6 | 1.6 |
| astrocytoma SW 1783 | 0.0 | 0.1 | 1.5 | 1.0 | 0.3 |
| neuro*; met SK-N-AS | 0.0 | 0.0 | 0.0 | 0.0 | 0.0 |
| astrocytoma SF-539 | 5.5 | 4.2 | 28.9 | 15.3 | 21.8 |
| astrocytoma SNB-75 | 1.9 | 1.3 | 10.7 | 5.4 | 5.5 |
| glioma SNB-19 | 0.6 | 1.6 | 2.4 | 1.7 | 3.6 |
| glioma U251 | 0.4 | 0.9 | 10.0 | 7.1 | 5.0 |
| glioma SF-295 | 11.3 | 12.1 | 24.1 | 17.1 | 25.5 |
| Heart (fetal) | 12.9 | 8.2 | 29.3 | 24.5 | 31.4 |
| Heart | 1.9 | 2.0 | 12.8 | 8.5 | 12.2 |
| Skeletal muscle (fetal) | 54.7 | 48.6 | 30.6 | 34.9 | 36.1 |
| Skeletal muscle | 1.0 | 0.6 | 3.1 | 4.4 | 3.2 |
| Bone marrow | 3.2 | 2.6 | 3.3 | 3.5 | 3.6 |
| Thymus | 4.9 | 5.3 | 10.2 | 11.1 | 11.3 |
| Spleen | 15.9 | 15.8 | 11.2 | 10.7 | 15.3 |
| Lymph node | 11.4 | 11.0 | 27.0 | 29.5 | 28.7 |
| Colorectal | 4.5 | 6.5 | 9.5 | 8.1 | 7.5 |
| Stomach | 9.2 | 8.5 | 9.5 | 8.2 | 9.7 |
| Small intestine | 5.7 | 6.4 | 6.8 | 4.6 | 5.7 |
| Colon ca. SW480 | 2.0 | 4.7 | 7.3 | 6.3 | 7.9 |
| Colon ca.* SW620(SW480 met) | 0.8 | 2.1 | 12.2 | 26.4 | 19.2 |
| Colon ca. HT29 | 0.3 | 0.4 | 5.2 | 4.2 | 4.3 |
| Colon ca. HCT-116 | 4.9 | 6.5 | 12.2 | 14.7 | 14.2 |
| Colon ca. CaCo-2 | 15.1 | 12.4 | 30.8 | 28.5 | 29.7 |
| Colon ca. tissue(ODO3866) | 5.9 | 5.6 | 17.3 | 24.3 | 19.3 |
| Colon ca. HCC-2998 | 5.0 | 9.9 | 30.8 | 31.9 | 35.8 |
| Gastric ca.* (liver met) NCI-N87 | 3.3 | 6.0 | 6.1 | 7.4 | 6.2 |
| Bladder | 2.1 | 1.9 | 14.3 | 9.7 | 15.7 |
| Trachea | 9.7 | 5.7 | 3.0 | 2.3 | 2.3 |
| Kidney | 5.5 | 3.9 | 24.0 | 23.2 | 21.9 |
| Kidney (fetal) | 11.8 | 14.4 | 100.0 | 100.0 | 85.9 |
| Renal ca. 786-0 | 6.5 | 5.2 | 27.4 | 28.3 | 33.2 |
| Renal ca. A498 | 8.2 | 9.6 | 19.3 | 21.5 | 21.8 |
| Renal ca. RXF 393 | 3.5 | 3.6 | 50.0 | 55.9 | 48.6 |
| Renal ca. ACHN | 3.2 | 4.7 | 8.7 | 7.6 | 9.2 |
| Renal ca. UO-31 | 3.0 | 50.0 | 3.3 | 4.2 | 4.2 |
| Renal ca. TK-10 | 4.4 | 4.9 | 18.0 | 13.7 | 15.5 |
| Liver | 3.2 | 1.2 | 5.6 | 4.4 | 8.0 |
| Liver (fetal) | 2.7 | 2.5 | 1.9 | 5.6 | 2.6 |
| Liver ca. (hepatoblast) HepG2 | 1.9 | 3.0 | 10.8 | 8.7 | 9.6 |
| Lung | 7.5 | 7.7 | 12.8 | 13.9 | 17.0 |
| Lung (fetal) | 7.0 | 8.2 | 13.3 | 6.8 | 5.0 |
| Lung ca. (small cell) LX-1 | 2.6 | 2.3 | 9.0 | 6.1 | 9.6 |
| Lung ca. (small cell) NCI-H69 | 0.7 | 0.3 | 1.5 | 1.0 | 0.5 |
| Lung ca. (s. cell var.) SHP-77 | 0.2 | 0.0 | 0.0 | 0.0 | 0.5 |
| Lung ca. (large cell)NCI-H460 | 0.5 | 1.1 | 0.6 | 0.0 | 0.7 |
| Lung ca. (non-sm. cell) A549 | 0.3 | 0.2 | 4.0 | 4.0 | 5.7 |
| Lung ca. (non-s. cell) NCI-H23 | 4.0 | 3.3 | 8.4 | 6.6 | 7.8 |
| Lung ca. (non-s. cell) HOP-62 | 1.5 | 1.7 | 1.5 | 2.4 | 3.3 |
| Lung ca. (non-s. cl) NCI-H522 | 4.0 | 8.4 | 13.4 | 12.3 | 12.0 |
| Lung ca. (squam.) SW 900 | 2.0 | 0.4 | 5.9 | 5.8 | 4.6 |
| Lung ca. (squam.) NCI-H596 | 0.0 | 0.0 | 0.0 | 0.4 | 1.1 |
| Mammary gland | 12.3 | 7.6 | 32.1 | 26.2 | 32.1 |
| Breast ca.* (pl. ef) MCF-7 | 15.3 | 10.5 | 76.3 | 79.0 | 100.0 |
| Breast ca.* (pl. ef) MDA-MB-231 | 1.8 | 2.0 | 6.2 | 6.7 | 6.5 |
| Breast ca.* (pl. ef) T47D | 4.5 | 6.4 | 35.8 | 31.9 | 37.4 |
| Breast ca. BT-549 | 0.9 | 1.4 | 9.1 | 6.3 | 6.2 |
| Breast ca. MDA-N | 0.6 | 0.9 | 2.9 | 4.3 | 6.5 |
| Ovary | 2.2 | 4.0 | 5.0 | 6.3 | 6.3 |
| Ovarian ca. OVCAR-3 | 8.7 | 6.9 | 26.2 | 31.6 | 41.2 |
| Ovarian ca. OVCAR-4 | 1.6 | 1.9 | 23.8 | 11.5 | 20.2 |
| Ovarian ca. OVCAR-5 | 1.9 | 2.6 | 20.7 | 17.6 | 14.7 |
| Ovarian ca. OVCAR-8 | 0.9 | 1.8 | 2.5 | 2.7 | 1.3 |
| Ovarian ca. IGROV-1 | 1.9 | 1.0 | 10.7 | 8.1 | 9.9 |
| Ovarian ca.* (ascites) SK-OV-3 | 0.5 | 1.2 | 16.7 | 12.0 | 10.7 |
| Uterus | 1.9 | 3.4 | 2.4 | 4.2 | 4.1 |
| Placenta | 1.6 | 1.6 | 1.8 | 1.5 | 1.4 |
| Prostate | 7.6 | 3.2 | 4.6 | 2.9 | 3.8 |
| Prostate ca.* (bone met)PC-3 | 6.5 | 7.0 | 19.9 | 17.2 | 19.3 |
| Testis | 4.0 | 3.3 | 3.0 | 1.0 | 1.9 |
| Melanoma Hs688(A).T | 0.0 | 0.0 | 0.0 | 0.0 | 0.0 |
| Melanoma* (met) Hs688(B).T | 0.0 | 0.0 | 0.0 | 0.0 | 0.0 |
| Melanoma UACC-62 | 0.2 | 0.2 | 1.0 | 3.6 | 3.8 |
| Melanoma M14 | 0.0 | 0.0 | 0.0 | 0.0 | 0.0 |
| Melanoma LOX IMVI | 0.5 | 0.4 | 0.0 | 0.0 | 0.0 |
| Melanoma* (met) SK-MEL-5 | 1.1 | 0.2 | 1.3 | 2.2 | 2.4 |
| Adipose | 9.7 | 11.0 | 32.8 | 29.9 | 31.4 |

Column A - Rel. Exp. (%) Ag1845, Run 148648438
Column B - Rel. Exp. (%) Ag1845, Run 149951496
Column C - Rel. Exp. (%) Ag2589, Run 167660070
Column D - Rel. Exp. (%) Ag2621, Run 167644903
Column E - Rel. Exp. (%) Ag2915, Run 167646705

TABLE AII
Panel 2D

| Tissue Name | A | B |
|---|---|---|
| Normal Colon | 35.1 | 24.5 |
| CC Well to Mod Diff (ODO3866) | 19.3 | 16.6 |
| CC Margin (ODO3866) | 9.4 | 11.9 |
| CC Gr.2 rectosigmoid (ODO3868) | 14.0 | 11.7 |
| CC Margin (ODO3868) | 1.4 | 1.9 |
| CC Mod Diff (ODO3920) | 9.6 | 7.1 |
| CC Margin (ODO3920) | 6.1 | 8.0 |
| CC Gr.2 ascend colon (ODO3921) | 58.2 | 47.6 |
| CC Margin (ODO3921) | 16.3 | 8.2 |
| CC from Partial Hepatectomy | 28.5 | 22.5 |
| (ODO4309) Mets | | |
| Liver Margin (ODO4309) | 18.4 | 8.1 |
| Colon mets to lung (OD04451-01) | 12.0 | 15.7 |
| Lung Margin (OD04451-02) | 6.5 | 3.9 |
| Normal Prostate 6546-1 | 23.2 | 17.3 |
| Prostate Cancer (OD04410) | 29.5 | 12.6 |
| Prostate Margin (OD04410) | 18.3 | 12.7 |
| Prostate Cancer (OD04720-01) | 39.0 | 28.9 |
| Prostate Margin (OD04720-02) | 32.8 | 32.3 |
| Normal Lung 061010 | 47.3 | 42.9 |
| Lung Met to Muscle (ODO4286) | 16.5 | 19.6 |
| Muscle Margin (ODO4286) | 3.5 | 4.5 |
| Lung Malignant Cancer (OD03126) | 25.3 | 26.2 |
| Lung Margin (OD03126) | 9.5 | 12.3 |
| Lung Cancer (OD04404) | 19.8 | 13.9 |
| Lung Margin (OD04404) | 12.3 | 10.1 |
| Lung Cancer (OD04565) | 4.8 | 3.8 |
| Lung Margin (OD04565) | 13.7 | 8.3 |
| Lung Cancer (OD04237-01) | 11.0 | 9.9 |
| Lung Margin (OD04237-02) | 21.0 | 23.0 |
| Ocular Mel Met to Liver (ODO4310) | 4.9 | 3.5 |
| Liver Margin (ODO4310) | 5.2 | 4.0 |
| Melanoma Mets to Lung (OD04321) | 1.3 | 3.5 |
| Lung Margin (OD04321) | 18.2 | 23.7 |
| Normal Kidney | 65.5 | 50.3 |
| Kidney Ca, Nuclear grade 2 (OD04338) | 49.0 | 36.1 |
| Kidney Margin (OD04338) | 48.3 | 33.2 |
| Kidney Ca Nuclear grade 1/2 (OD04339) | 19.8 | 14.0 |
| Kidney Margin (OD04339) | 81.8 | 65.1 |
| Kidney Ca, Clear cell type (OD04340) | 100.0 | 100.0 |
| Kidney Margin (OD04340) | 82.4 | 66.0 |
| Kidney Ca, Nuclear grade 3 (OD04348) | 9.9 | 9.9 |
| Kidney Margin (OD04348) | 45.1 | 42.6 |
| Kidney Cancer (OD04622-01) | 24.3 | 14.2 |
| Kidney Margin (OD04622-03) | 9.4 | 10.0 |
| Kidney Cancer (OD04450-01) | 6.5 | 6.2 |
| Kidney Margin (OD04450-03) | 22.4 | 33.0 |
| Kidney Cancer 8120607 | 33.4 | 24.5 |
| Kidney Margin 8120608 | 44.4 | 28.3 |
| Kidney Cancer 8120613 | 30.4 | 17.8 |
| Kidney Margin 8120614 | 62.9 | 51.8 |
| Kidney Cancer 9010320 | 36.3 | 26.4 |
| Kidney Margin 9010321 | 46.7 | 38.2 |
| Normal Uterus | 2.1 | 3.8 |
| Uterus Cancer 064011 | 17.8 | 14.5 |
| Normal Thyroid | 8.7 | 5.0 |
| Thyroid Cancer 064010 | 10.2 | 8.9 |
| Thyroid Cancer A302152 | 2.9 | 4.5 |
| Thyroid Margin A302153 | 10.3 | 7.3 |
| Normal Breast | 18.0 | 17.1 |
| Breast Cancer (OD04566) | 15.7 | 11.5 |
| Breast Cancer (OD04590-01) | 42.6 | 49.7 |
| Breast Cancer Mets (OD04590-03) | 42.6 | 31.2 |
| Breast Cancer Metastasis (OD04655-05) | 33.4 | 23.3 |
| Breast Cancer 064006 | 15.6 | 15.3 |
| Breast Cancer 1024 | 64.2 | 49.0 |
| Breast Cancer 9100266 | 12.1 | 12.2 |
| Breast Margin 9100265 | 17.7 | 20.0 |
| Breast Cancer A209073 | 27.5 | 33.2 |
| Breast Margin A209073 | 22.8 | 27.7 |
| Normal Liver | 14.4 | 8.1 |
| Liver Cancer 064003 | 4.9 | 4.1 |
| Liver Cancer 1025 | 9.3 | 8.3 |
| Liver Cancer 1026 | 8.4 | 8.8 |
| Liver Cancer 6004-T | 10.2 | 11.9 |
| Liver Tissue 6004-N | 2.5 | 2.0 |
| Liver Cancer 6005-T | 7.3 | 6.2 |
| Liver Tissue 6005-N | 2.9 | 3.2 |
| Normal Bladder | 17.3 | 15.2 |
| Bladder Cancer 1023 | 11.0 | 12.3 |
| Bladder Cancer A302173 | 46.3 | 47.0 |
| Bladder Cancer (OD04718-01) | 65.1 | 42.3 |
| Bladder Normal Adjacent (OD04718-03) | 11.6 | 6.7 |
| Normal Ovary | 2.9 | 2.3 |
| Ovarian Cancer 064008 | 32.5 | 28.1 |
| Ovarian Cancer (OD04768-07) | 9.3 | 8.0 |
| Ovary Margin (OD04768-08) | 8.8 | 6.3 |
| Normal Stomach | 15.4 | 16.3 |
| Gastric Cancer 9060358 | 10.4 | 4.0 |
| Stomach Margin 9060359 | 15.6 | 20.6 |
| Gastric Cancer 9060395 | 25.9 | 26.4 |
| Stomach Margin 9060394 | 35.8 | 29.9 |
| Gastric Cancer 9060397 | 79.0 | 64.2 |
| Stomach Margin 9060396 | 10.9 | 8.4 |
| Gastric Cancer 064005 | 23.8 | 27.5 |

Column A - Rel. Exp. (%) Ag1845, Run 148648439
Column B - Rel. Exp. (%) Ag1845, Run 149957753

TABLE AIJ
Panel 4.1D

| Tissue Name | A |
|---|---|
| Secondary Th1 act | 6.4 |
| Secondary Th2 act | 3.5 |
| Secondary Tr1 act | 0.6 |
| Secondary Th1 rest | 0.0 |
| Secondary Th2 rest | 0.0 |
| Secondary Tr1 rest | 0.0 |
| Primary Th1 act | 0.8 |
| Primary Th2 act | 6.3 |
| Primary Tr1 act | 5.1 |
| Primary Th1 rest | 0.0 |
| Primary Th2 rest | 0.0 |
| Primary Tr1 rest | 0.0 |
| CD45RA CD4 lymphocyte act | 2.6 |
| CD45RO CD4 lymphocyte act | 2.1 |
| CD8 lymphocyte act | 0.0 |
| Secondary CD8 lymphocyte rest | 0.0 |
| Secondary CD8 lymphocyte act | 0.8 |
| CD4 lymphocyte none | 0.4 |
| 2ry Th1/Th2/Tr1_anti-CD95 CH11 | 0.7 |
| LAK cells rest | 0.9 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.2 |
| LAK cells PMA/ionomycin | 1.2 |
| NK Cells IL-2 rest | 0.4 |
| Two Way MLR 3 day | 2.1 |
| Two Way MLR 5 day | 0.2 |
| Two Way MLR 7 day | 0.8 |
| PBMC rest | 2.2 |
| PBMC PWM | 1.3 |
| PBMC PHA-L | 0.0 |
| Ramos (B cell) none | 6.3 |
| Ramos (B cell) ionomycin | 7.8 |
| B lymphocytes PWM | 0.3 |
| B lymphocytes CD40L and IL-4 | 3.1 |
| EOL-1 dbcAMP | 36.3 |
| EOL-1 dbcAMP PMA/ionomycin | 47.0 |
| Dendritic cells none | 9.2 |
| Dendritic cells LPS | 2.8 |
| Dendritic cells anti-CD40 | 0.4 |
| Monocytes rest | 0.4 |
| Monocytes LPS | 5.5 |
| Macrophages rest | 1.4 |
| Macrophages LPS | 2.7 |
| HUVEC none | 0.3 |
| HUVEC starved | 1.0 |
| HUVEC IL-1beta | 2.2 |
| HUVEC IFN gamma | 1.2 |
| HUVEC INF alpha + IFN gamma | 2.8 |
| HUVEC TNF alpha + IL4 | 2.9 |
| HUVEC IL-11 | 0.0 |
| Lung Microvascular EC none | 1.3 |
| Lung Microvascular EC TNFalpha + IL-1beta | 11.8 |
| Microvascular Dermal EC none | 0.0 |
| Microsvasular Dermal EC TNFalpha + IL-1beta | 4.0 |
| Bronchial epithelium TNFalpha + IL1beta | 0.3 |
| Small airway epithelium none | 0.0 |
| Small airway epithelium TNFalpha + IL-1beta | 1.0 |
| Coronery artery SMC rest | 0.7 |
| Coronery artery SMC TNFalpha + IL-1beta | 1.2 |
| Astrocytes rest | 0.0 |
| Astrocytes TNFalpha + IL-1beta | 14.0 |
| KU-812 (Basophil) rest | 2.5 |
| KU-812 (Basophil) PMA/ionomycin | 1.3 |
| CCD1106 (Keratinocytes) none | 0.0 |
| CCD1106 (Keratinocytes) TNFalpha + IL-1beta | 0.2 |
| Liver cirrhosis | 0.4 |
| NCI-H292 none | 0.2 |
| NCI-H292 IL-4 | 0.5 |
| NCI-H292 IL-9 | 0.2 |
| NCI-H292 IL-13 | 0.8 |
| NCI-H292 IFN gamma | 0.5 |
| HPAEC none | 0.7 |
| HPAEC TNF alpha + IL-1 beta | 100.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 6.4 |
| Dermal fibroblast CCD1070 IL-1 beta | 0.6 |
| Dermal fibroblast IFN gamma | 0.0 |
| Dermal fibroblast IL-4 | 0.0 |
| Dermal Fibroblasts rest | 0.0 |
| Neutrophils TNFa + LPS | 0.9 |
| Neutrophils rest | 0.0 |
| Colon | 0.0 |
| Lung | 0.0 |
| Thymus | 1.0 |
| Kidney | 2.7 |

Column A - Rel. Exp.(%) Ag8156, Run 319806134

TABLE AIK
general oncology screening panel_v_2.4

| Tissue Name | A |
|---|---|
| Colon cancer 1 | 26.6 |
| Colon NAT 1 | 5.8 |
| Colon cancer 2 | 7.1 |
| Colon NAT 2 | 3.6 |
| Colon cancer 3 | 26.6 |
| Colon NAT 3 | 7.3 |
| Colon malignant cancer 4 | 23.8 |
| Colon NAT 4 | 1.5 |
| Lung cancer 1 | 5.8 |
| Lung NAT 1 | 1.3 |
| Lung cancer 2 | 42.6 |
| Lung NAT 2 | 1.7 |
| Squamous cell carcinoma 3 | 10.0 |
| Lung NAT 3 | 0.6 |
| Metastatic melanoma 1 | 3.1 |
| Melanoma 2 | 6.4 |
| Melanoma 3 | 3.3 |
| Metastatic melanoma 4 | 4.6 |
| Metastatic melanoma 5 | 6.0 |
| Bladder cancer 1 | 0.3 |
| Bladder NAT 1 | 0.0 |
| Bladder cancer 2 | 0.8 |
| Bladder NAT 2 | 0.2 |
| Bladder NAT 3 | 0.3 |
| Bladder NAT 4 | 3.8 |
| Prostate adenocarcinoma 1 | 4.4 |
| Prostate adenocarcinoma 2 | 0.9 |
| Prostate adenocarcinoma 3 | 3.4 |
| Prostate adenocarcinoma 4 | 8.0 |
| Prostate NAT 5 | 2.2 |
| Prostate adenocarcinoma 6 | 0.8 |
| Prostate adenocarcinoma 7 | 2.9 |
| Prostate adenocarcinoma 8 | 0.4 |
| Prostate adenocarcinoma 9 | 12.4 |
| Prostate NAT 10 | 0.4 |
| Kidney cancer 1 | 24.0 |
| Kidney NAT 1 | 9.6 |
| Kidney cancer 2 | 100.0 |
| Kidney NAT 2 | 13.5 |
| Kidney cancer 3 | 31.0 |
| Kidney NAT 3 | 11.8 |
| Kidney cancer 4 | 27.0 |
| Kidney NAT 4 | 13.7 |

Column A - Rel. Exp. (%)Ag2915, Run 260443054

AI_comprehensive panel_v1.0 Summary: Ag1845 This transcript was expressed at low levels in many different disease tissues. In comparison, normal lung and joint tissues expressed none or extremely low levels of this transcript. Since this transcript was expressed in monocytes, and matched control tissues contain these inflammatory cells (psoriasis, Crohn's and ulcerative colitis), this transcript is detected at these sites. This transcript encodes B7-H2, which has been shown to be important in antigen presentation. It is a ligand for ICOS and serves as a costimulatory molecule. Therapeutics designed with the transcript are useful for the reduction or inhibition of antigen presentation and in the treatment of diseases such as asthma, IBD, psoriasis and arthritis in which T cells are chronically stimulated.

CNS_neurodegeneration_v1.0 Summary: Ag1845/Ag2589/Ag2621/Ag2915 The expression of this gene was up-regulated in the temporal cortex of Alzheimer's disease patients when compared to non-demented controls. This difference was apparent when the data were analyzed via ANCOVA, using overall RNA quality and/or quantity as a covariate. The up-regulation of this gene was most apparent in the variant detected by Ag1845. The temporal cortex is a region that shows degeneration at the mid-stages of this disease. Thus, the phenomenon of neurodegeneration was captured in this region, as opposed to the hippocampus and entorhinal cortex where a large number of neurons are already lost by the time of death in AD. Furthermore, in the occipital cortex (where neurodegeneration does not occur in Alzheimer's) this gene was not found to be up-regulated in the same patients. Taken together, these data demonstrate that this gene is useful as a marker of Alzheimer's-like neurodegeneration, and is involved in the process of neurodegeneration. This gene is a form of the B7 protein (B7-H2B), which plays a role in inflammation. Neuroinflammation has been implicated in AD, to the extent that long-term usage of anti-inflammatory agents has been correlated with a reduced incidence of Alzheimer's in retrospective studies. This gene therefore represents an excellent drug target for the treatment of Alzheimer's disease, and any other neuroinflammatory condition.

Panel 1.3D Summary: Ag1845/Ag2589/Ag2621/Ag2915 Highest expression of this gene was seen in the brain, fetal kidney, and a breast cancer cell line. Higher levels of expression were also consistently seen in fetal skeletal muscle (CTs=29-30), when compared to expression in adult skeletal muscle (CTs=33-35). Thus, expression of this gene is useful as a marker to differentiate between the adult and fetal sources of this tissue. The relative overexpression of this gene in fetal sekeltal muscle shows that the protein product enhances muscle growth or development in the fetus and also acts in a regenerative capacity in the adult. Therapeutic modulation of the protein encoded by this gene is useful in the treatment of lung related diseases.

This gene product was also moderately expressed in pancreas, adrenal, thyroid, pituitary, adult and fetal liver, adult and fetal heart, and adipose. Based on its expression profile in metabolic tissues, this gene product is useful in the diagnosis and/or treatment of metabolic disease, including obesity and diabetes.

Panel 2D Summary: Ag1845 The expression of this gene was highest in a sample derived from a kidney cancer (CTs=28). There was substantial expression associated with other samples derived from kidney tissue, bladder cancer and breast cancer. The expression of this gene is useful as a marker to distinguish this kidney cancer sample from other samples in the panel. Therapeutic modulation of this gene, through the use of small molecule drugs, protein therapeutics or antibodies is beneficial for the treatment of kidney cancer, breast cancer or bladder cancer.

Panel 4.1D Summary: Ag8156 Highest expression of this gene was detected in TNF alpha+IL-1 beta activated HPAEC cells (CT=30.9). Low expression of this gene was also seen in activated lung microvascular endothelial cells. Expression of this gene was very low or undetectable in resting endothelial cells. Low expression of this gene was also seen in activated astrocytes, eosinophils, Ramos B cells, and activated primary Th2 and secondary Th1 cells. This gene codes for B7-H2B gene. B7-H2 is a recently cloned costimulatory ligand that belongs to B7 family. B7-H2 binds to ICOS, an inducible costimulatory receptor expressed on activated, but not resting T cells. (Wang S, Blood 2000 October 15;96(8):2808-13) Interaction of B7-H2 and ICOS in the presence of the TCR stimuli results in an augmented proliferative responses and cytokine production. The data show that circumstances B7-H2 more effectively costimulates Th2 responses. The B7-H2/ICOS pathway plays an important role in antibody response and germinal center formation. (Tesciuba. J Immunol 2001 August 15;167(4):1996-2003) Thus, B7-H2 is useful in the generation of therapeutics for diseases with antibody-mediated and Th2-mediated pathogenesis. Also, antibodies and small molecules that antagonize the function of the B7-H2B protein are useful for the reduction or elimination of the symptoms in patients with autoimmune and inflammatory diseases that involve endothelial cells, such as lupus erythematosus, asthma, emphysema, Crohn's disease, ulcerative colitis, rheumatoid arthritis, osteoarthritis, and psoriasis.

general oncology screening panel_v_{—}2.4 Summary: Ag2915 Highest expression of this gene was detected in a kidney cancer sample (CT=28.2). Moderate to low expression of this gene was also seen in cancer and normal samples derived from kidney, colon, metastatic melanoma, prostate and lung. Expression of this gene was higher in cancer samples. Therefore, expression of this gene is useful as a diagnostic marker to detect the presence of these cancers. Therapeutic modulation of this gene or its protein product through the use of antibodies or small molecule drug is useful in the treatment of kidney, colon, metastatic melanoma, prostate and lung cancers.

AJ. CG55906-01: S3-12

Expression of gene CG55906-01 was assessed using the primer-probe sets Ag2840 and Ag6179, described in Tables AJA and AJB. Results of the RTQ-PCR runs are shown in Tables AJC, AMD, AME, AJF and AJG.

TABLE AJA
Probe Name Ag2840

| | | | | |
|---|---|---|---|---|
| | | | Start | SEQ ID |
| Primers | Sequences | Length | Position | No |

| | | | | |
|---|---|---|---|---|
| Forward | 5′-tctatggtcatgggtacgaaag-3′ | 22 | 1190 | 1444 |
| Probe | TET-5′-acacgatgtccactgggctcacag-3′- | 24 | 1212 | 1445 |
| | TAMRA | | | |
| Reverse | 5′-gttgtgttcagcccagtttg-3′ | 20 | 1265 | 1446 |

TABLE AJB
Probe Name Ag6179

| | | | | |
|---|---|---|---|---|
| | | | Start | SEQ ID |
| Primers | Sequences | Length | Position | No |

| | | | | |
|---|---|---|---|---|
| Forward | 5′-ttctcactatgtctgctcca-3′ | 20 | 123 | 1447 |
| Probe | TET-5′-atgcagaccctgggcagcttct-3′-TAMRA | 22 | 194 | 1448 |
| Reverse | 5′-ttccgggcagagctgaa-3′ | 17 | 233 | 1449 |

TABLE AJC
General_screening_panel_v1.5

| Tissue Name | A |
|---|---|
| Adipose | 73.7 |
| Melanoma* Hs688(A).T | 0.0 |
| Melanoma* Hs688(B).T | 0.0 |
| Melanoma* M14 | 0.0 |
| Melanoma* LOXIMVI | 0.2 |
| Melanoma* SK-MEL-5 | 0.0 |
| Squamous cell carcinoma SCC-4 | 0.0 |
| Testis Pool | 0.9 |
| Prostate ca.* (bone met) PC-3 | 5.1 |
| Prostate Pool | 2.6 |
| Placenta | 0.1 |
| Uterus Pool | 1.3 |
| Ovarian ca. OVCAR-3 | 0.3 |
| Ovarian ca. SK-OV-3 | 0.0 |
| Ovarian ca. OVCAR-4 | 0.0 |
| Ovarian ca. OVCAR-5 | 3.4 |
| Ovarian ca. IGROV-1 | 0.1 |
| Ovarian ca. OVCAR-8 | 0.3 |
| Ovary | 0.7 |
| Breast ca. MCF-7 | 0.1 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 6.7 |
| Trachea | 5.0 |
| Lung | 0.2 |
| Fetal Lung | 0.1 |
| Lung ca. NCI-N417 | 0.0 |
| Lung ca. LX-1 | 0.1 |
| Lung ca. NCI-H146 | 0.0 |
| Lung ca. SHP-77 | 0.1 |
| Lung ca. A549 | 0.0 |
| Lung ca. NCI-H526 | 0.0 |
| Lung ca. NCI-H23 | 0.1 |
| Lung ca. NCI-H460 | 0.0 |
| Lung ca. HOP-62 | 0.0 |
| Lung ca. NCI-H522 | 0.0 |
| Liver | 0.8 |
| Fetal Liver | 0.9 |
| Liver ca. HepG2 | 1.4 |
| Kidney Pool | 13.7 |
| Fetal Kidney | 0.1 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 1.0 |
| Bladder | 6.5 |
| Gastric ca. (liver met.) NCI-N87 | 1.7 |
| Gastric ca. KATO III | 0.3 |
| Colon ca. SW-948 | 0.1 |
| Colon ca. SW480 | 0.0 |
| Colon ca.* (SW480 met) SW620 | 0.0 |
| Colon ca. HT29 | 0.8 |
| Colon ca. HCT-116 | 0.4 |
| Colon ca. CaCo-2 | 0.1 |
| Colon cancer tissue | 17.6 |
| Colon ca. SW1116 | 0.0 |
| Colon ca. Colo-205 | 0.0 |
| Colon ca. SW-48 | 0.0 |
| Colon Pool | 7.7 |
| Small Intestine Pool | 6.1 |
| Stomach Pool | 3.7 |
| Bone Marrow Pool | 1.7 |
| Fetal Heart | 0.8 |
| Heart Pool | 7.9 |
| Lymph Node Pool | 9.1 |
| Fetal Skeletal Muscle | 2.5 |
| Skeletal Muscle Pool | 100.0 |
| Spleen Pool | 0.0 |
| Thymus Pool | 5.9 |
| CNS cancer (glio/astro) U87-MG | 0.0 |
| CNS cancer (glio/astro) U-118-MG | 0.1 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.3 |
| Brain (Amygdala) Pool | 0.8 |
| Brain (cerebellum) | 0.7 |
| Brain (fetal) | 0.1 |
| Brain (Hippocampus) Pool | 0.8 |
| Cerebral Cortex Pool | 0.5 |
| Brain (Substantia nigra) Pool | 0.9 |
| Brain (Thalamus) Pool | 0.8 |
| Brain (whole) | 0.2 |
| Spinal Cord Pool | 3.5 |
| Adrenal Gland | 3.7 |
| Pituitary gland Pool | 0.0 |
| Salivary Gland | 1.8 |
| Thyroid (female) | 0.5 |
| Pancreatic ca. CAPAN2 | 1.0 |
| Pancreas Pool | 1.8 |

Column A - Rel. Exp.(%) Ag6179, Run 256555247

TABLE AJD
Panel 2D

| Tissue Name | A |
|---|---|
| Normal Colon | 41.8 |
| CC Well to Mod Diff (ODO3866) | 4.3 |
| CC Margin (ODO3866) | 6.9 |
| CC Gr.2 rectosigmoid (ODO3868) | 1.1 |
| CC Margin (ODO3868) | 4.5 |
| CC Mod Diff (ODO3920) | 0.0 |
| CC Margin (ODO3920) | 0.0 |
| CC Gr.2 ascend colon (ODO3921) | 0.4 |
| CC Margin (ODO3921) | 2.5 |
| CC from Partial Hepatectomy (ODO4309) | 0.2 |
| Mets | |
| Liver Margin (ODO4309) | 1.8 |
| Colon mets to lung (OD04451-01) | 0.1 |
| Lung Margin (OD04451-02) | 0.1 |
| Normal Prostate 6546-1 | 0.9 |
| Prostate Cancer (OD04410) | 1.2 |
| Prostate Margin (OD04410) | 3.2 |
| Prostate Cancer (OD04720-01) | 0.8 |
| Prostate Margin (OD04720-02) | 1.9 |
| Normal Lung 061010 | 1.4 |
| Lung Met to Muscle (ODO4286) | 0.5 |
| Muscle Margin (ODO4286) | 16.4 |
| Lung Malignant Cancer (OD03126) | 0.1 |
| Lung Margin (OD03126) | 0.4 |
| Lung Cancer (OD04404) | 0.3 |
| Lung Margin (OD04404) | 0.3 |
| Lung Cancer (OD04565) | 0.0 |
| Lung Margin (OD04565) | 0.2 |
| Lung Cancer (OD04237-01) | 0.0 |
| Lung Margin (OD04237-02) | 0.1 |
| Ocular Mel Met to Liver (ODO4310) | 0.4 |
| Liver Margin (ODO4310) | 2.6 |
| Melanoma Mets to Lung (OD04321) | 0.0 |
| Lung Margin (OD04321) | 0.2 |
| Normal Kidney | 3.1 |
| Kidney Ca, Nuclear grade 2 (OD04338) | 0.0 |
| Kidney Margin (OD04338) | 0.0 |
| Kidney Ca Nuclear grade 1/2 (OD04339) | 0.2 |
| Kidney Margin (OD04339) | 1.1 |
| Kidney Ca, Clear cell type (OD04340) | 0.3 |
| Kidney Margin (OD04340) | 1.8 |
| Kidney Ca, Nuclear grade 3 (OD04348) | 0.0 |
| Kidney Margin (OD04348) | 1.3 |
| Kidney Cancer (OD04622-01) | 0.0 |
| Kidney Margin (OD04622-03) | 0.0 |
| Kidney Cancer (OD04450-01) | 0.1 |
| Kidney Margin (OD04450-03) | 1.2 |
| Kidney Cancer 8120607 | 0.1 |
| Kidney Margin 8120608 | 0.2 |
| Kidney Cancer 8120613 | 0.7 |
| Kidney Margin 8120614 | 2.0 |
| Kidney Cancer 9010320 | 8.4 |
| Kidney Margin 9010321 | 2.0 |
| Normal Uterus | 2.4 |
| Uterus Cancer 064011 | 4.4 |
| Normal Thyroid | 2.3 |
| Thyroid Cancer 064010 | 0.0 |
| Thyroid Cancer A302152 | 0.4 |
| Thyroid Margin A302153 | 0.5 |
| Normal Breast | 80.7 |
| Breast Cancer (OD04566) | 1.5 |
| Breast Cancer (OD04590-01) | 21.0 |
| Breast Cancer Mets (OD04590-03) | 100.0 |
| Breast Cancer Metastasis (OD04655-05) | 13.3 |
| Breast Cancer 064006 | 0.9 |
| Breast Cancer 1024 | 5.8 |
| Breast Cancer 9100266 | 3.2 |
| Breast Margin 9100265 | 5.9 |
| Breast Cancer A209073 | 2.0 |
| Breast Margin A209073 | 7.9 |
| Normal Liver | 3.3 |
| Liver Cancer 064003 | 2.2 |
| Liver Cancer 1025 | 6.0 |
| Liver Cancer 1026 | 2.0 |
| Liver Cancer 6004-T | 7.5 |
| Liver Tissue 6004-N | 2.2 |
| Liver Cancer 6005-T | 1.8 |
| Liver Tissue 6005-N | 0.3 |
| Normal Bladder | 6.0 |
| Bladder Cancer 1023 | 0.7 |
| Bladder Cancer A302173 | 0.4 |
| Bladder Cancer (OD04718-01) | 0.1 |
| Bladder Normal Adjacent (OD04718-03) | 11.7 |
| Normal Ovary | 0.8 |
| Ovarian Cancer 064008 | 1.1 |
| Ovarian Cancer (OD04768-07) | 0.7 |
| Ovary Margin (OD04768-08) | 2.9 |
| Normal Stomach | 15.1 |
| Gastric Cancer 9060358 | 6.4 |
| Stomach Margin 9060359 | 2.0 |
| Gastric Cancer 9060395 | 8.8 |
| Stomach Margin 9060394 | 8.4 |
| Gastric Cancer 9060397 | 0.9 |
| Stomach Margin 9060396 | 3.1 |
| Gastric Cancer 064005 | 5.3 |

Column A - Rel. Exp.(%) Ag2840, Run 161922469

TABLE AJE
Panel 3D

| Tissue Name | A |
|---|---|
| Daoy- Medulloblastoma | 22.1 |
| TE671- Medulloblastoma | 10.1 |
| D283 Med- Medulloblastoma | 2.3 |
| PFSK-1- Primitive Neuroectodermal | 6.2 |
| XF-498- CNS | 8.4 |
| SNB-78- Glioma | 5.0 |
| SF-268- Glioblastoma | 1.4 |
| T98G- Glioblastoma | 0.0 |
| SK-N-SH- Neuroblastoma (metastasis) | 2.7 |
| SF-295- Glioblastoma | 2.9 |
| Cerebellum | 3.4 |
| Cerebellum | 38.2 |
| NCI-H292- Mucoepidermoid lung | 0.0 |
| carcinoma | |
| DMS-114- Small cell lung cancer | 0.0 |
| DMS-79- Small cell lung cancer | 100.0 |
| NCI-H146- Small cell lung cancer | 0.0 |
| NCI-H526- Small cell lung cancer | 9.6 |
| NCI-N417- Small cell lung cancer | 0.0 |
| NCI-H82- Small cell lung cancer | 3.3 |
| NCI-H157- Squamous cell lung cancer | 0.0 |
| (metastasis) | |
| NCI-H1155- Large cell lung cancer | 20.6 |
| NCI-H1299- Large cell lung cancer | 9.2 |
| NCI-H727- Lung carcinoid | 0.0 |
| NCI-UMC-11- Lung carcinoid | 0.0 |
| LX-1- Small cell lung cancer | 7.9 |
| Colo-205- Colon cancer | 0.0 |
| KM12- Colon cancer | 0.0 |
| KM20L2- Colon cancer | 2.9 |
| NCI-H716- Colon cancer | 0.0 |
| SW-48- Colon adenocarcinoma | 0.0 |
| SW1116- Colon adenocarcinoma | 0.0 |
| LS 174T- Colon adenocarcinoma | 0.0 |
| SW-948- Colon adenocarcinoma | 0.0 |
| SW-480- Colon adenocarcinoma | 3.8 |
| NCI-SNU-5- Gastric carcinoma | 2.1 |
| KATO III- Gastric carcinoma | 12.0 |
| NCI-SNU-16- Gastric carcinoma | 5.8 |
| NCI-SNU-1- Gastric carcinoma | 2.6 |
| RF-1- Gastric adenocarcinoma | 0.0 |
| RF-48- Gastric adenocarcinoma | 7.2 |
| MKN-45- Gastric carcinoma | 96.6 |
| NCI-N87- Gastric carcinoma | 10.0 |
| OVCAR-5- Ovarian carcinoma | 3.7 |
| RL95-2- Uterine carcinoma | 7.2 |
| HelaS3- Cervical adenocarcinoma | 0.0 |
| Ca Ski- Cervical epidermoid carcinoma | 7.6 |
| (metastasis) | |
| ES-2- Ovarian clear cell carcinoma | 0.0 |
| Ramos- Stimulated with PMA/ionomycin 6 h | 0.0 |
| Ramos- Stimulated with PMA/ionomycin 14 h | 0.0 |
| MEG-01- Chronic myelogenous leukemia | 0.0 |
| (megokaryoblast) | |
| Raji- Burkitt's lymphoma | 0.0 |
| Daudi- Burkitt's lymphoma | 0.0 |
| U266- B-cell plasmacytoma | 10.4 |
| CA46- Burkitt's lymphoma | 0.0 |
| RL- non-Hodgkin's B-cell lymphoma | 5.9 |
| JM1- pre-B-cell lymphoma | 7.8 |
| Jurkat- T cell leukemia | 2.7 |
| TF-1- Erythroleukemia | 5.0 |
| HUT 78- T-cell lymphoma | 0.0 |
| U937- Histiocytic lymphoma | 11.0 |
| KU-812- Myelogenous leukemia | 31.0 |
| 769-P- Clear cell renal carcinoma | 6.1 |
| Caki-2- Clear cell renal carcinoma | 3.0 |
| SW 839- Clear cell renal carcinoma | 0.0 |
| Rhabdoid kidney tumor | 3.7 |
| Hs766T- Pancreatic carcinoma (LN | 9.8 |
| metastasis) | |
| CAPAN-1- Pancreatic adenocarcinoma (liver | 0.0 |
| metastasis) | |
| SU86.86- Pancreatic carcinoma (liver | 5.0 |
| metastasis) | |
| BxPC-3- Pancreatic adenocarcinoma | 13.0 |
| HPAC- Pancreatic adenocarcinoma | 20.7 |
| MIA PaCa-2- Pancreatic carcinoma | 4.2 |
| CFPAC-1- Pancreatic ductal adenocarcinoma | 13.9 |
| PANC-1- Pancreatic epithelioid ductal | 69.7 |
| carcinoma | |
| T24- Bladder carcinma (transitional cell) | 0.0 |
| 5637- Bladder carcinoma | 0.0 |
| HT-1197- Bladder carcinoma | 6.4 |
| UM-UC-3- Bladder carcinma (transitional cell) | 2.4 |
| A204- Rhabdomyosarcoma | 12.5 |
| HT-1080- Fibrosarcoma | 12.1 |
| MG-63- Osteosarcoma | 0.0 |
| SK-LMS-1- Leiomyosarcoma (vulva) | 16.7 |
| SJRH30- Rhabdomyosarcoma (met to bone | 5.7 |
| marrow) | |
| A431- Epidermoid carcinoma | 0.0 |
| WM266-4- Melanoma | 0.0 |
| DU 145- Prostate carcinoma (brain | 0.0 |
| metastasis) | |
| MDA-MB-468- Breast adenocarcinoma | 11.4 |
| SCC-4- Squamous cell carcinoma of tongue | 0.0 |
| SCC-9- Squamous cell carcinoma of tongue | 0.0 |
| SCC-15- Squamous cell carcinoma of tongue | 0.0 |
| CAL 27- Squamous cell carcinoma of tongue | 0.0 |

Column A - Rel. Exp.(%) Ag2840, Run 170190088

TABLE AJF
Panel 4.1D

| Tissue Name | A |
|---|---|
| Secondary Th1 act | 6.4 |
| Secondary Th2 act | 2.0 |
| Secondary Tr1 act | 4.0 |
| Secondary Th1 rest | 4.4 |
| Secondary Th2 rest | 13.9 |
| Secondary Tr1 rest | 8.2 |
| Primary Th1 act | 3.0 |
| Primary Th2 act | 9.6 |
| Primary Tr1 act | 1.6 |
| Primary Th1 rest | 3.2 |
| Primary Th2 rest | 5.6 |
| Primary Tr1 rest | 6.4 |
| CD45RA CD4 lymphocyte act | 6.0 |
| CD45RO CD4 lymphocyte act | 6.2 |
| CD8 lymphocyte act | 6.4 |
| Secondary CD8 lymphocyte rest | 2.4 |
| Secondary CD8 lymphocyte act | 0.0 |
| CD4 lymphocyte none | 12.5 |
| 2ry Th1/Th2/Tr1_anti-CD95 CH11 | 11.3 |
| LAK cells rest | 7.6 |
| LAK cells IL-2 | 4.2 |
| LAK cells IL-2 + IL-12 | 1.9 |
| LAK cells IL-2 + IFN gamma | 2.7 |
| LAK cells IL-2 + IL-18 | 6.6 |
| LAK cells PMA/ionomycin | 0.0 |
| NK Cells IL-2 rest | 3.4 |
| Two Way MLR 3 day | 6.7 |
| Two Way MLR 5 day | 5.8 |
| Two Way MLR 7 day | 8.5 |
| PBMC rest | 9.3 |
| PBMC PWM | 1.0 |
| PBMC PHA-L | 3.2 |
| Ramos (B cell) none | 1.9 |
| Ramos (B cell) ionomycin | 3.0 |
| B lymphocytes PWM | 1.0 |
| B lymphocytes CD40L and IL-4 | 9.4 |
| EOL-1 dbcAMP | 0.8 |
| EOL-1 dbcAMP PMA/ionomycin | 6.0 |
| Dendritic cells none | 0.9 |
| Dendritic cells LPS | 0.0 |
| Dendritic cells anti-CD40 | 3.1 |
| Monocytes rest | 13.8 |
| Monocytes LPS | 4.9 |
| Macrophages rest | 2.1 |
| Macrophages LPS | 0.9 |
| HUVEC none | 6.3 |
| HUVEC starved | 7.9 |
| HUVEC IL-1beta | 0.9 |
| HUVEC IFN gamma | 4.0 |
| HUVEC TNF alpha + IFN gamma | 0.0 |
| HUVEC TNF alpha + IL4 | 5.4 |
| HUVEC IL-11 | 3.8 |
| Lung Microvascular EC none | 8.4 |
| Lung Microvascular EC TNFalpha + IL-1beta | 4.0 |
| Microvascular Dermal EC none | 4.5 |
| Microsvasular Dermal EC TNFalpha + IL-1beta | 0.0 |
| Bronchial epithelium TNFalpha + IL1beta | 10.4 |
| Small airway epithelium none | 5.9 |
| Small airway epithelium TNFalpha + IL-1beta | 8.5 |
| Coronery artery SMC rest | 0.0 |
| Coronery artery SMC TNFalpha + IL-1beta | 1.0 |
| Astrocytes rest | 0.0 |
| Astrocytes TNFalpha + IL-1beta | 1.4 |
| KU-812 (Basophil) rest | 8.4 |
| KU-812 (Basophil) PMA/ionomycin | 6.1 |
| CCD1106 (Keratinocytes) none | 6.7 |
| CCD1106 (Keratinocytes) TNFalpha + IL-1beta | 1.8 |
| Liver cirrhosis | 63.3 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 0.0 |
| HPAEC TNF alpha + IL-1 beta | 3.3 |
| Lung fibroblast none | 29.5 |
| Lung fibroblast TNF alpha + IL-1 beta | 8.2 |
| Lung fibroblast IL-4 | 7.9 |
| Lung fibroblast IL-9 | 10.3 |
| Lung fibroblast IL-13 | 4.2 |
| Lung fibroblast IFN gamma | 9.1 |
| Dermal fibroblast CCD1070 rest | 2.3 |
| Dermal fibroblast CCD1070 TNF alpha | 9.2 |
| Dermal fibroblast CCD1070 IL-1beta | 4.9 |
| Dermal fibroblast IFN gamma | 1.0 |
| Dermal fibroblast IL-4 | 8.1 |
| Dermal Fibroblasts rest | 4.2 |
| Neutrophils TNFa + LPS | 25.0 |
| Neutrophils rest | 66.9 |
| Colon | 100.0 |
| Lung | 3.8 |
| Thymus | 24.5 |
| Kidney | 32.1 |

Column A - Rel. Exp.(%) Ag2840, Run 204964146

TABLE AJG
Panel 5 Islet

| Tissue Name | A |
|---|---|
| 97457_Patient-02go_adipose | 100.0 |
| 97476_Patient-07sk_skeletal muscle | 22.8 |
| 97477_Patient-07ut_uterus | 13.8 |
| 97478_Patient-07pl_placenta | 0.0 |
| 99167_Bayer Patient 1 | 1.0 |
| 97482_Patient-08ut_uterus | 6.1 |
| 97483_Patient-08pl_placenta | 0.0 |
| 97486_Patient-09sk_skeletal muscle | 2.3 |
| 97487_Patient-09ut_uterus | 5.5 |
| 97488_Patient-09pl_placenta | 0.0 |
| 97492_Patient-10ut_uterus | 4.4 |
| 97493_Patient-10pl_placenta | 0.0 |
| 97495_Patient-11go_adipose | 24.3 |
| 97496_Patient-11sk_skeletal muscle | 13.0 |
| 97497_Patient-11ut_uterus | 11.7 |
| 97498_Patient-11pl_placenta | 0.0 |
| 97500_Patient-12go_adipose | 78.5 |
| 97501_Patient-12sk_skeletal muscle | 63.3 |
| 97502_Patient-12ut_uterus | 13.0 |
| 97503_Patient-12pl_placenta | 0.0 |
| 94721_Donor 2 U - A_Mesenchymal | 0.0 |
| Stem Cells | |
| 94722_Donor 2 U - B_Mesenchymal | 0.0 |
| Stem Cells | |
| 94723_Donor 2 U - C_Mesenchymal | 0.0 |
| Stem Cells | |
| 94709_Donor 2 AM - A_adipose | 18.9 |
| 94710_Donor 2 AM - B_adipose | 12.5 |
| 94711_Donor 2 AM - C_adipose | 15.2 |
| 94712_Donor 2 AD - A_adipose | 25.2 |
| 94713_Donor 2 AD - B_adipose | 30.4 |
| 94714_Donor 2 AD - C_adipose | 42.3 |
| 94742_Donor 3 U - A_Mesenchymal Stem | 0.0 |
| Cells | |
| 94743_Donor 3 U - B_Mesenchymal Stem | 0.0 |
| Cells | |
| 94730_Donor 3 AM - A_adipose | 12.6 |
| 94731_Donor 3 AM - B_adipose | 7.9 |
| 94732_Donor 3 AM - C_adipose | 11.0 |
| 94733_Donor 3 AD - A_adipose | 26.8 |
| 94734_Donor 3 AD - B_adipose | 18.0 |
| 94735_Donor 3 AD - C_adipose | 17.1 |
| 77138_Liver_HepG2untreated | 0.3 |
| 73556_Heart_Cardiac stromal cells | 0.0 |
| (primary) | |
| 81735_Small Intestine | 12.9 |
| 72409_Kidney_Proximal Convoluted Tubule | 0.0 |
| 82685_Small intestine_Duodenum | 1.0 |
| 90650_Adrenal_Adrenocortical adenoma | 0.0 |
| 72410_Kidney_HRCE | 0.0 |
| 72411_Kidney_HRE | 0.0 |
| 73139_Uterus_Uterine smooth muscle cells | 0.0 |

Column A - Rel. Exp.(%) Ag6179, Run 256791278

General_screening_panel_v1.5 Summary: Ag6179 Highest expression of this gene was detected in skeletal muscle (CT=28.6). Moderate levels of expression of this gene are also seen in adipose, colon, small intestine and heart. This gene codes for a homolog of mouse S3-12 protein. S3-12 is a purported adipose cell membrane-associated protein that is upregulated during adipocyte differentiation. Due to its weak homology with adipophilins, this gene is involved in lipid uptake. Inhibiting its action by an antibody is useful for reducing the potential white adipose mass by limiting lipid uptake and inhibiting adipocyte differentiation. Its expression in skeletal muscle shows that it can function similarly in muscle tissue as well, in terms of lipid uptake. Since excess lipid storage in muscle is associated with insulin resistance, antibody inhibition of this gene product is useful as a treatment for the prevention of obesity-associated insulin resistance.

Furthermore, this gene product was also moderately expressed in a variety of metabolic tissues, including adrenal gland, heart, colon and small intestine. Thus, modulation of this gene or expressed protein is useful as marker for the treatment of metabolic disease, including Types 1 and 2 diabetes, and obesity.

In addition, moderate but significant expression in the spinal cordshows that the protein encoded by this gene plays a role in lipid processing in the central nervous system. LDLR has been implicated in the development of Alzheimer's disease. Therefore, modulation of this gene or gene product is useful in the treatment of Alzheimer's Disease.

Panel 2D Summary: Ag2840 This gene was moderately expressed in all tissue samples this panel, with highest expression in a metastatic breast cancer sample (CT=24.5). This gene showed higher expression in normal kidney, prostate, colon and bladder samples relative to the corresponding adjacent tumor sample. Expression of this gene is useful as a marker to differentiate between normal and cancerous tissues. Therapeutic modulation of this gene, expressed protein and/or use of agonists targeting this gene or gene product is useful in the treatment of kidney, prostate, colon and bladder cancers.

Panel 3D Summary: Ag2840 This gene showed low levels of expression in cancer cell lines, with highest expression in a gastric cancer MKN-45 cell line (CT=32). Significant expression was seen in cancer cell lines derived from lung cancer, pancreatic cancer and a leukemia. Therapeutic modulation of this gene, expressed protein and/or use of antibodies or small molecule drug targeting this gene or gene product is useful in the treatment of these cancers.

Panel 4.1D Summary: Ag2840 This transcript is expressed in colon and in resting neutrophils (CTs=31-33). Thus, the transcript or the protein it encodes is useful as a marker to detect colon tissue and neutrophils. Therapeutics modulation of this gene, expressed protein and/or use of antibodies or small molecule targeting this gene or gene product are useful in the treatment of autoimmune and inflammatory diseases such as asthma, allergies, inflammatory bowel disease including Crohn's and Colitis, lupus erythematosus, psoriasis, rheumatoid arthritis, and osteoarthritis.

Panel 5 Islet Summary: Ag6179 Highest expression of this gene was detected in adipose tissue from a diabetic patient. Significant expression of this gene was also seen in midway and fully differentiated adipose tissue. This gene was moderately expressed in clinical specimens of adipose, skeletal muscle and uterus.

AK. CG5590602 and CG5590604 and CG5590605: Human Ortholog of Mouse S3-12

Expression of full-length physical clone CG55906-02 and variants CG55906-04 and CG55906-05 was assessed using the primer-probe sets Ag2840, Ag5976 and Ag6345, described in Tables AKA, AKB and AKC. Results of the RTQ-PCR runs are shown in Tables AKD, AKE, AKF, AKG and AKH. Please note that only the probe and primer set Ag6345 corresponds to CG55906-02 and only probe and primer sets Ag5976 and Ag6345 correspond to CG55906-05.

TABLE AKA
Probe Name Ag2840

| | | | | |
|---|---|---|---|---|
| | | | Start | SEQ ID |
| Primers | Sequences | Length | Position | No |

| | | | | |
|---|---|---|---|---|
| Forward | 5′-tctatggtcatgggtacgaaag-3′ | 22 | 1183 | 1450 |
| Probe | TET-5′-acacgatgtccactgggctcacag-3′- | 24 | 1205 | 1451 |
| | TAMRA | | | |
| Reverse | 5′-gttgtgttcagcccagtttg-3′ | 20 | 1258 | 1452 |

TABLE AKB
Probe Name Ag5976

| | | | | |
|---|---|---|---|---|
| | | | Start | SEQ ID |
| Primers | Sequences | Length | Position | No |

| | | | | |
|---|---|---|---|---|
| Forward | 5′-caacttcggaaaagcagat-3′ | 20 | 244 | 1453 |
| Probe | TET-5′-ctggacatcttggaacacaccaggtc-3′- | 26 | 280 | 1454 |
| | TAMRA | | | |
| Reverse | 5′-ggaggacacggcatcct-3′ | 17 | 311 | 1455 |

TABLE AKC
Probe Name Ag6345

| | | | | |
|---|---|---|---|---|
| | | | Start | SEQ ID |
| Primers | Sequences | Length | Position | No |

| | | | | |
|---|---|---|---|---|
| Forward | 5′-aacgcacatagctcggcga-3′ | 19 | 112 | 1456 |
| Probe | TET-5′-tgaccccacaggagcgcctgct-3′-TAMRA | 22 | 147 | 1457 |
| Reverse | 5′-aaggttgcagctccttctccgt-3′ | 22 | 229 | 1458 |

TABLE AKD
General_screening_panel_v1.5

| Tissue Name | A | B |
|---|---|---|
| Adipose | 100.0 | 0.0 |
| Melanoma* Hs688(A).T | 0.0 | 0.0 |
| Melanoma* Hs688(B).T | 0.0 | 0.0 |
| Melanoma* M14 | 0.0 | 0.0 |
| Melanoma* LOXIMVI | 0.0 | 0.0 |
| Melanoma* SK-MEL-5 | 0.0 | 0.0 |
| Squamous cell carcinoma SCC-4 | 0.0 | 0.0 |
| Testis Pool | 0.6 | 1.6 |
| Prostate ca.* (bone met) PC-3 | 3.2 | 3.6 |
| Prostate Pool | 1.4 | 1.3 |
| Placenta | 0.0 | 0.0 |
| Uterus Pool | 2.8 | 0.9 |
| Ovarian ca. OVCAR-3 | 0.0 | 3.0 |
| Ovarian ca. SK-OV-3 | 0.0 | 0.0 |
| Ovarian ca. OVCAR-4 | 0.1 | 0.0 |
| Ovarian ca. OVCAR-5 | 4.3 | 7.9 |
| Ovarian ca. IGROV-1 | 0.0 | 0.0 |
| Ovarian ca. OVCAR-8 | 0.0 | 0.0 |
| Ovary | 0.5 | 0.7 |
| Breast ca. MCF-7 | 0.3 | 0.0 |
| Breast ca. MDA-MB-231 | 0.0 | 0.0 |
| Breast ca. BT 549 | 0.0 | 0.0 |
| Breast ca. T47D | 0.0 | 0.0 |
| Breast ca. MDA-N | 0.0 | 0.0 |
| Breast Pool | 8.2 | 6.7 |
| Trachea | 7.3 | 1.5 |
| Lung | 0.3 | 0.0 |
| Fetal Lung | 0.4 | 0.9 |
| Lung ca. NCI-N417 | 0.0 | 0.0 |
| Lung ca. LX-1 | 0.1 | 0.0 |
| Lung ca. NCI-H146 | 0.0 | 0.0 |
| Lung ca. SHP-77 | 0.0 | 0.0 |
| Lung ca. A549 | 0.0 | 0.0 |
| Lung ca. NCI-H526 | 0.0 | 0.0 |
| Lung ca. NCI-H23 | 0.0 | 0.0 |
| Lung ca. NCI-H460 | 0.0 | 0.0 |
| Lung ca. HOP-62 | 0.0 | 0.0 |
| Lung ca. NCI-H522 | 0.0 | 0.0 |
| Liver | 0.7 | 0.0 |
| Fetal Liver | 1.2 | 1.7 |
| Liver ca. HepG2 | 2.0 | 1.2 |
| Kidney Pool | 15.5 | 41.8 |
| Fetal Kidney | 0.1 | 0.0 |
| Renal ca. 786-0 | 0.0 | 0.0 |
| Renal ca. A498 | 0.0 | 0.0 |
| Renal ca. ACHN | 0.0 | 0.0 |
| Renal ca. UO-31 | 0.0 | 0.0 |
| Renal ca. TK-10 | 0.3 | 0.0 |
| Bladder | 3.4 | 0.0 |
| Gastric ca. (liver met.) NCI-N87 | 2.2 | 3.9 |
| Gastric ca. KATO III | 0.1 | 1.7 |
| Colon ca. SW-948 | 0.0 | 0.0 |
| Colon ca. SW480 | 0.0 | 0.0 |
| Colon ca.* (SW480 met) SW620 | 0.0 | 0.0 |
| Colon ca. HT29 | 1.9 | 1.4 |
| Colon ca. HCT-116 | 0.0 | 0.0 |
| Colon ca. CaCo-2 | 0.3 | 0.0 |
| Colon cancer tissue | 15.4 | 15.5 |
| Colon ca. SW1116 | 0.0 | 0.0 |
| Colon ca. Colo-205 | 0.0 | 0.0 |
| Colon ca. SW-48 | 0.0 | 0.0 |
| Colon Pool | 9.4 | 7.0 |
| Small Intestine Pool | 4.8 | 1.4 |
| Stomach Pool | 6.1 | 6.0 |
| Bone Marrow Pool | 3.8 | 2.6 |
| Fetal Heart | 0.2 | 0.0 |
| Heart Pool | 12.2 | 5.0 |
| Lymph Node Pool | 10.4 | 13.9 |
| Fetal Skeletal Muscle | 2.0 | 0.0 |
| Skeletal Muscle Pool | 54.7 | 100.0 |
| Spleen Pool | 0.0 | 0.0 |
| Thymus Pool | 6.2 | 5.5 |
| CNS cancer (glio/astro) U87-MG | 0.0 | 0.0 |
| CNS cancer (glio/astro) U-118-MG | 0.0 | 0.0 |
| CNS cancer (neuro; met) SK-N-AS | 0.0 | 0.3 |
| CNS cancer (astro) SF-539 | 0.0 | 0.0 |
| CNS cancer (astro) SNB-75 | 0.0 | 0.0 |
| CNS cancer (glio) SNB-19 | 0.0 | 0.0 |
| CNS cancer (glio) SF-295 | 0.5 | 0.0 |
| Brain (Amygdala) Pool | 0.5 | 2.7 |
| Brain (cerebellum) | 0.4 | 0.0 |
| Brain (fetal) | 0.3 | 0.0 |
| Brain (Hippocampus) Pool | 0.0 | 2.4 |
| Cerebral Cortex Pool | 1.0 | 1.1 |
| Brain (Substantia nigra) Pool | 0.6 | 0.9 |
| Brain (Thalamus) Pool | 0.9 | 0.0 |
| Brain (whole) | 0.4 | 0.0 |
| Spinal Cord Pool | 2.5 | 2.9 |
| Adrenal Gland | 4.2 | 1.6 |
| Pituitary gland Pool | 0.4 | 0.0 |
| Salivary Gland | 3.0 | 3.1 |
| Thyroid (female) | 0.6 | 0.0 |
| Pancreatic ca. CAPAN2 | 1.6 | 0.6 |
| Pancreas Pool | 8.5 | 18.2 |

Column A - Rel. Exp. (%) Ag5976, Run 248204740
Column B - Rel. Exp. (%) Ag6345, Run 259476291

TABLE AKE
Panel 2D

| Tissue Name | A |
|---|---|
| Normal Colon | 41.8 |
| CC Well to Mod Diff (ODO3866) | 4.3 |
| CC Margin (ODO3866) | 6.9 |
| CC Gr.2 rectosigmoid (ODO3868) | 1.1 |
| CC Margin (ODO3868) | 4.5 |
| CC Mod Diff (ODO3920) | 0.0 |
| CC Margin (ODO3920) | 0.0 |
| CC Gr.2 ascend colon (ODO3921) | 0.4 |
| CC Margin (ODO3921) | 2.5 |
| CC from Partial Hepatectomy (ODO4309) Mets | 0.2 |
| Liver Margin (ODO4309) | 1.8 |
| Colon mets to lung (OD04451-01) | 0.1 |
| Lung Margin (OD04451-02) | 0.1 |
| Normal Prostate 6546-1 | 0.9 |
| Prostate Cancer (OD04410) | 1.2 |
| Prostate Margin (OD04410) | 3.2 |
| Prostate Cancer (OD04720-01) | 0.8 |
| Prostate Margin (OD04720-02) | 1.9 |
| Normal Lung 061010 | 1.4 |
| Lung Met to Muscle (ODO4286) | 0.5 |
| Muscle Margin (ODO4286) | 16.4 |
| Lung Malignant Cancer (OD03126) | 0.1 |
| Lung Margin (OD03126) | 0.4 |
| Lung Cancer (OD04404) | 0.3 |
| Lung Margin (OD04404) | 0.3 |
| Lung Cancer (OD04565) | 0.0 |
| Lung Margin (OD04565) | 0.2 |
| Lung Cancer (OD04237-01) | 0.0 |
| Lung Margin (OD04237-02) | 0.1 |
| Ocular Mel Met to Liver (ODO4310) | 0.4 |
| Liver Margin (ODO4310) | 2.6 |
| Melanoma Mets to Lung (OD04321) | 0.0 |
| Lung Margin (OD04321) | 0.2 |
| Normal Kidney | 3.1 |
| Kidney Ca, Nuclear grade 2 (OD04338) | 0.0 |
| Kidney Margin (OD04338) | 0.0 |
| Kidney Ca Nuclear grade 1/2 (OD04339) | 0.2 |
| Kidney Margin (OD04339) | 1.1 |
| Kidney Ca, Clear cell type (OD04340) | 0.3 |
| Kidney Margin (OD04340) | 1.8 |
| Kidney Ca, Nuclear grade 3 (OD04348) | 0.0 |
| Kidney Margin (OD04348) | 1.3 |
| Kidney Cancer (OD04622-01) | 0.0 |
| Kidney Margin (OD04622-03) | 0.0 |
| Kidney Cancer (OD04450-01) | 0.1 |
| Kidney Margin (OD04450-03) | 1.2 |
| Kidney Cancer 8120607 | 0.10 |
| Kidney Margin 8120608 | 0.2 |
| Kidney Cancer 8120613 | 0.7 |
| Kidney Margin 8120614 | 2.0 |
| Kidney Cancer 9010320 | 8.4 |
| Kidney Margin 9010321 | 2.0 |
| Normal Uterus | 2.4 |
| Uterus Cancer 064011 | 4.4 |
| Normal Thyroid | 2.3 |
| Thyroid Cancer 064010 | 0.0 |
| Thyroid Cancer A302152 | 0.4 |
| Thyroid Margin A302153 | 0.5 |
| Normal Breast | 80.7 |
| Breast Cancer (OD04566) | 1.5 |
| Breast Cancer (OD04590-01) | 21.0 |
| Breast Cancer Mets (OD04590-03) | 100.0 |
| Breast Cancer Metastasis (OD04655-05) | 13.3 |
| Breast Cancer 064006 | 0.9 |
| Breast Cancer 1024 | 5.8 |
| Breast Cancer 9100266 | 3.2 |
| Breast Margin 9100265 | 5.9 |
| Breast Cancer A209073 | 2.0 |
| Breast Margin A209073 | 7.9 |
| Normal Liver | 3.3 |
| Liver Cancer 064003 | 2.2 |
| Liver Cancer 1025 | 6.0 |
| Liver Cancer 1026 | 2.0 |
| Liver Cancer 6004-T | 7.5 |
| Liver Tissue 6004-N | 2.2 |
| Liver Cancer 6005-T | 1.8 |
| Liver Tissue 6005-N | 0.3 |
| Normal Bladder | 6.0 |
| Bladder Cancer 1023 | 0.7 |
| Bladder Cancer A302173 | 0.4 |
| Bladder Cancer (OD04718-01) | 0.1 |
| Bladder Normal Adjacent (OD04718-03) | 11.7 |
| Normal Ovary | 0.8 |
| Ovarian Cancer 064008 | 1.1 |
| Ovarian Cancer (OD04768-07) | 0.7 |
| Ovary Margin (OD04768-08) | 2.9 |
| Normal Stomach | 15.1 |
| Gastric Cancer 9060358 | 6.4 |
| Stomach Margin 9060359 | 2.0 |
| Gastric Cancer 9060395 | 8.8 |
| Stomach Margin 9060394 | 8.4 |
| Gastric Cancer 9060397 | 0.9 |
| Stomach Margin 9060396 | 3.1 |
| Gastric Cancer 064005 | 5.3 |

Column A - Rel. Exp. (%) Ag2840, Run 161922469

TABLE AKF
Panel 3D

| Tissue Name | A |
|---|---|
| Daoy- Medulloblastoma | 22.1 |
| TE671- Medulloblastoma | 10.1 |
| D283 Med- Medulloblastoma | 2.3 |
| PFSK-1- Primitive Neuroectodermal | 6.2 |
| XF-498- CNS | 8.4 |
| SNB-78- Glioma | 5.0 |
| SF-268- Glioblastoma | 1.4 |
| T98G- Glioblastoma | 0.0 |
| SK-N-SH- Neuroblastoma (metastasis) | 2.7 |
| SF-295- Glioblastoma | 2.9 |
| Cerebellum | 3.4 |
| Cerebellum | 38.2 |
| NCI-H292- Mucoepidermoid lung carcinoma | 0.0 |
| DMS-114- Small cell lung cancer | 0.0 |
| DMS-79- Small cell lung cancer | 100.0 |
| NCI-H146- Small cell lung cancer | 0.0 |
| NCI-H526- Small cell lung cancer | 9.6 |
| NCI-N417- Small cell lung cancer | 0.0 |
| NCI-H82- Small cell lung cancer | 3.3 |
| NCI-H157- Squamous cell lung cancer (metastasis) | 0.0 |
| NCI-H1155- Large cell lung cancer | 20.6 |
| NCI-H1299- Large cell lung cancer | 9.2 |
| NCI-H727- Lung carcinoid | 0.0 |
| NCI-UMC-11- Lung carcinoid | 0.0 |
| LX-1- Small cell lung cancer | 7.9 |
| Colo-205- Colon cancer | 0.0 |
| KM12- Colon cancer | 0.0 |
| KM20L2- Colon cancer | 2.9 |
| NCI-H716- Colon cancer | 0.0 |
| SW-48- Colon adenocarcinoma | 0.0 |
| SW1116- Colon adenocarcinoma | 0.0 |
| LS 174T- Colon adenocarcinoma | 0.0 |
| SW-948- Colon adenocarcinoma | 0.0 |
| SW-480- Colon adenocarcinoma | 3.8 |
| NCI-SNU-5- Gastric carcinoma | 2.1 |
| KATO III- Gastric carcinoma | 12.0 |
| NCI-SNU-16- Gastric carcinoma | 5.8 |
| NCI-SNU-1- Gastric carcinoma | 2.6 |
| RF-1- Gastric adenocarcinoma | 0.0 |
| RF-48- Gastric adenocarcinoma | 7.2 |
| MKN-45- Gastric carcinoma | 96.6 |
| NCI-N87- Gastric carcinoma | 10.0 |
| OVCAR-5- Ovarian carcinoma | 3.7 |
| RL95-2- Uterine carcinoma | 7.2 |
| HelaS3- Cervical adenocarcinoma | 0.0 |
| Ca Ski- Cervical epidermoid carcinoma (metastasis) | 7.6 |
| ES-2- Ovarian clear cell carcinoma | 0.0 |
| Ramos- Stimulated with PMA/ionomycin 6 h | 0.0 |
| Ramos- Stimulated with PMA/ionomycin 14 h | 0.0 |
| MEG-01- Chronic myelogenous leukemia (megokaryoblast) | 0.0 |
| Raji- Burkitt's lymphoma | 0.0 |
| Daudi- Burkitt's lymphoma | 0.0 |
| U266- B-cell plasmacytoma | 10.4 |
| CA46- Burkitt's lymphoma | 0.0 |
| RL- non-Hodgkin's B-cell lymphoma | 5.9 |
| JM1- pre-B-cell lymphoma | 7.8 |
| Jurkat- T cell leukemia | 2.7 |
| TF-1- Erythroleukemia | 5.0 |
| HUT 78- T-cell lymphoma | 0.0 |
| U937- Histiocytic lymphoma | 11.0 |
| KU-812- Myelogenous leukemia | 31.0 |
| 769-P- Clear cell renal carcinoma | 6.1 |
| Caki-2- Clear cell renal carcinoma | 3.0 |
| SW 839- Clear cell renal carcinoma | 0.0 |
| Rhabdoid kidney tumor | 3.7 |
| Hs766T- Pancreatic carcinoma (LN metastasis) | 9.8 |
| CAPAN-1- Pancreatic adenocarcinoma (liver metastasis) | 0.0 |
| SU86.86- Pancreatic carcinoma (liver metastasis) | 5.0 |
| BxPC-3- Pancreatic adenocarcinoma | 13.0 |
| HPAC- Pancreatic adenocarcinoma | 20.7 |
| MIA PaCa-2- Pancreatic carcinoma | 4.2 |
| CFPAC-1- Pancreatic ductal adenocarcinoma | 13.9 |
| PANC-1- Pancreatic epithelioid ductal carcinoma | 69.7 |
| T24- Bladder carcinma (transitional cell) | 0.0 |
| 5637- Bladder carcinoma | 0.0 |
| HT-1197- Bladder carcinoma | 6.4 |
| UM-UC-3- Bladder carcinma (transitional cell) | 2.4 |
| A204- Rhabdomyosarcoma | 12.5 |
| HT-1080- Fibrosarcoma | 12.1 |
| MG-63- Osteosarcoma | 0.0 |
| SK-LMS-1- Leiomyosarcoma (vulva) | 16.7 |
| SJRH30- Rhabdomyosarcoma (met to bone marrow) | 5.7 |
| A431- Epidermoid carcinoma | 0.0 |
| WM266-4- Melanoma | 0.0 |
| DU 145- Prostate carcinoma (brain metastasis) | 0.0 |
| MDA-MB-468- Breast adenocarcinoma | 11.4 |
| SCC-4- Squamous cell carcinoma of tongue | 0.0 |
| SCC-9- Squamous cell carcinoma of tongue | 0.0 |
| SCC-15- Squamous cell carcinoma of tongue | 0.0 |
| CAL 27- Squamous cell carcinoma of tongue | 0.0 |

Column A - Rel. Exp. (%) Ag2840, Run 170190088

TABLE AKG
Panel 4.1D

| Tissue Name | A | B |
|---|---|---|
| Secondary Th1 act | 6.4 | 0.0 |
| Secondary Th2 act | 2.0 | 0.0 |
| Secondary Tr1 act | 4.0 | 0.0 |
| Secondary Th1 rest | 4.4 | 0.0 |
| Secondary Th2 rest | 13.9 | 0.0 |
| Secondary Tr1 rest | 8.2 | 0.0 |
| Primary Th1 act | 3.0 | 0.0 |
| Primary Th2 act | 9.6 | 0.0 |
| Primary Tr1 act | 1.6 | 0.0 |
| Primary Th1 rest | 3.2 | 0.0 |
| Primary Th2 rest | 5.6 | 0.0 |
| Primary Tr1 rest | 6.4 | 0.0 |
| CD45RA CD4 lymphocyte act | 6.0 | 0.0 |
| CD45RO CD4 lymphocyte act | 6.2 | 0.0 |
| CD8 lymphocyte act | 6.4 | 0.0 |
| Secondary CD8 lymphocyte rest | 2.4 | 0.0 |
| Secondary CD8 lymphocyte act | 0.0 | 0.0 |
| CD4 lymphocyte none | 12.5 | 0.0 |
| 2ry Th1/Th2/Tr1_anti-CD95 CH11 | 11.3 | 0.0 |
| LAK cells rest | 7.6 | 0.0 |
| LAK cells IL-2 | 4.2 | 0.0 |
| LAK cells IL-2 + IL-12 | 1.9 | 0.0 |
| LAK cells IL-2 + IFN gamma | 2.7 | 0.0 |
| LAK cells IL-2 + IL-18 | 6.6 | 0.0 |
| LAK cells PMA/ionomycin | 0.0 | 0.0 |
| NK Cells IL-2 rest | 3.4 | 0.0 |
| Two Way MLR 3 day | 6.7 | 0.0 |
| Two Way MLR 5 day | 5.8 | 0.0 |
| Two Way MLR 7 day | 8.5 | 0.0 |
| PBMC rest | 9.3 | 0.0 |
| PBMC PWM | 1.0 | 0.0 |
| PBMC PHA-L | 3.2 | 0.0 |
| Ramos (B cell) none | 1.9 | 0.0 |
| Ramos (B cell) ionomycin | 3.0 | 0.0 |
| B lymphocytes PWM | 1.0 | 0.0 |
| B lymphocytes CD40L and IL-4 | 9.4 | 0.0 |
| EOL-1 dbcAMP | 0.8 | 0.0 |
| EOL-1 dbcAMP PMA/ionomycin | 6.0 | 0.0 |
| Dendritic cells none | 0.9 | 0.0 |
| Dendritic cells LPS | 0.0 | 0.0 |
| Dendritic cells anti-CD40 | 3.1 | 0.0 |
| Monocytes rest | 13.8 | 0.0 |
| Monocytes LPS | 4.9 | 0.0 |
| Macrophages rest | 2.1 | 0.0 |
| Macrophages LPS | 0.9 | 0.0 |
| HUVEC none | 6.3 | 0.0 |
| HUVEC starved | 7.9 | 0.0 |
| HUVEC IL-1beta | 0.9 | 0.0 |
| HUVEC IFN gamma | 4.0 | 0.0 |
| HUVEC TNF alpha + IFN gamma | 0.0 | 0.0 |
| HUVEC TNF alpha + IL4 | 5.4 | 0.0 |
| HUVEC IL-11 | 3.8 | 0.0 |
| Lung Microvascular EC none | 8.4 | 0.0 |
| Lung Microvascular EC TNFalpha + IL-1beta | 4.0 | 0.0 |
| Microvascular Dermal EC none | 4.5 | 0.0 |
| Microsvasular Dermal EC TNFalpha + IL-1beta | 0.0 | 0.0 |
| Bronchial epithelium TNFalpha + IL1beta | 10.4 | 0.0 |
| Small airway epithelium none | 5.9 | 0.0 |
| Small airway epithelium TNFalpha + IL-1beta | 8.5 | 0.0 |
| Coronery artery SMC rest | 0.0 | 0.0 |
| Coronery artery SMC TNFalpha + IL-1beta | 1.0 | 0.0 |
| Astrocytes rest | 0.0 | 0.0 |
| Astrocytes TNFalpha + IL-1beta | 1.4 | 0.0 |
| KU-812 (Basophil) rest | 8.4 | 20.0 |
| KU-812 (Basophil) PMA/ionomycin | 6.1 | 0.0 |
| CCD1106 (Keratinocytes) none | 6.7 | 0.0 |
| CCD1106 (Keratinocytes) TNFalpha + IL-1beta | 1.8 | 0.0 |
| Liver cirrhosis | 63.3 | 0.0 |
| NCI-H292 none | 0.0 | 0.0 |
| NCI-H292 IL-4 | 0.0 | 56.3 |
| NCI-H292 IL-9 | 0.0 | 65.5 |
| NCI-H292 IL-13 | 0.0 | 48.6 |
| NCI-H292 IFN gamma | 0.0 | 24.3 |
| HPAEC none | 0.0 | 0.0 |
| HPAEC TNF alpha + IL-1 beta | 3.3 | 0.0 |
| Lung fibroblast none | 29.5 | 100.0 |
| Lung fibroblast TNF alpha + IL-1 beta | 8.2 | 0.0 |
| Lung fibroblast IL-4 | 7.9 | 0.0 |
| Lung fibroblast IL-9 | 10.3 | 0.0 |
| Lung fibroblast IL-13 | 4.2 | 0.0 |
| Lung fibroblast IFN gamma | 9.1 | 0.0 |
| Dermal fibroblast CCD1070 rest | 2.3 | 0.0 |
| Dermal fibroblast CCD1070 TNF alpha | 9.2 | 0.0 |
| Dermal fibroblast CCD1070 IL-1 beta | 4.9 | 0.0 |
| Dermal fibroblast IFN gamma | 1.0 | 0.0 |
| Dermal fibroblast IL-4 | 8.1 | 0.0 |
| Dermal Fibroblasts rest | 4.2 | 0.0 |
| Neutrophils TNFa + LPS | 25.0 | 0.0 |
| Neutrophils rest | 66.9 | 18.7 |
| Colon | 100.0 | 54.7 |
| Lung | 3.8 | 0.0 |
| Thymus | 24.5 | 46.7 |
| Kidney | 32.1 | 0.0 |

Column A - Rel. Exp. (%) Ag2840, Run 204964146
Column B - Rel. Exp. (%) Ag5976, Run 248122612

TABLE AKH
Panel 5D

| Tissue Name | A |
|---|---|
| 97457_Patient-02go_adipose | 63.3 |
| 97476_Patient-07sk_skeletal muscle | 15.8 |
| 97477_Patient-07ut_uterus | 8.8 |
| 97478_Patient-07pl_placenta | 0.0 |
| 97481_Patient-08sk_skeletal muscle | 15.7 |
| 97482_Patient-08ut_uterus | 8.4 |
| 97483_Patient-08pl_placenta | 0.0 |
| 97486_Patient-09sk_skeletal muscle | 1.4 |
| 97487_Patient-09ut_uterus | 5.5 |
| 97488_Patient-09pl_placenta | 0.0 |
| 97492_Patient-10ut_uterus | 4.6 |
| 97493_Patient-10pl_placenta | 0.3 |
| 97495_Patient-11go_adipose | 0.0 |
| 97496_Patient-11sk_skeletal muscle | 0.0 |
| 97497_Patient-11ut_uterus | 0.0 |
| 97498_Patient-11pl_placenta | 0.0 |
| 97500_Patient-12go_adipose | 48.0 |
| 97501_Patient-12sk_skeletal muscle | 100.0 |
| 97502_Patient-12ut_uterus | 18.7 |
| 97503_Patient-12pl_placenta | 0.0 |
| 94721_Donor 2 U - A_Mesenchymal Stem Cells | 0.0 |
| 94722_Donor 2 U - B_Mesenchymal Stem Cells | 0.2 |
| 94723_Donor 2 U - C_Mesenchymal Stem Cells | 0.0 |
| 94709_Donor 2 AM - A_adipose | 24.1 |
| 94710_Donor 2 AM - B_adipose | 7.9 |
| 94711_Donor 2 AM - C_adipose | 4.5 |
| 94712_Donor 2 AD - A_adipose | 18.3 |
| 94713_Donor 2 AD - B_adipose | 24.1 |
| 94714_Donor 2 AD - C_adipose | 23.5 |
| 94742_Donor 3 U - A_Mesenchymal Stem Cells | 0.0 |
| 94743_Donor 3 U - B_Mesenchymal Stem Cells | 0.0 |
| 94730_Donor 3 AM - A_adipose | 0.0 |
| 94731_Donor 3 AM - B_adipose | 0.0 |
| 94732_Donor 3 AM - C_adipose | 0.1 |
| 94733_Donor 3 AD - A_adipose | 0.0 |
| 94734_Donor 3 AD - B_adipose | 0.0 |
| 94735_Donor 3 AD - C_adipose | 0.0 |
| 77138_Liver_HepG2untreated | 1.1 |
| 73556_Heart_Cardiac stromal cells (primary) | 0.0 |
| 81735_Small Intestine | 16.8 |
| 72409_Kidney_Proximal Convoluted Tubule | 0.0 |
| 82685_Small intestine_Duodenum | 0.7 |
| 90650_Adrenal_Adrenocortical adenoma | 0.1 |
| 72410_Kidney_HRCE | 0.0 |
| 72411_Kidney_HRE | 0.0 |
| 73139_Uterus_Uterine smooth muscle cells | 0.2 |

Column A - Rel. Exp. (%) Ag2840, Run 169270970

General_screening_panel_v1.5 Summary: Ag5976 Highest expression of this gene was detected in adipose (CT=29). Moderate levels of expression of this gene was also seen in skeletal muscle, colon, small intestine, adrenal gland and heart. This gene codes for a homolog of mouse S3-12 protein. S3-12 is an adipose cell membrane-associated protein upregulated during adipocyte differentiation. Due to its weak homology with adipophilins, it is involved in lipid uptake. Inhibiting its action by an antibody will reduce potential white adipose mass by limiting lipid uptake and thereby inhibit adipocyte differentiation. Its expression in skeletal muscle indicates that it functions similarly in muscle tissue as well, in terms of lipid uptake. Since excess lipid storage in muscle is associated with insulin resistance, antibody inhibition of this gene product is useful as a treatment for the prevention of obesity-associated insulin resistance.

Furthermore, modulation of this gene or expressed protein is useful for the treatment of metabolic disease, including Types 1 and 2 diabetes, and obesity.

Low expression of this gene was also seen in colon cancer tissue and cancer cell lines derived from ovarian and prostate cancers.

Low levels of expression in the spinal cord show that the protein encoded by this gene plays a role in lipid processing in the central nervous system. Modulation of this gene or gene product is useful in the treatment of spinal cord related disorders.

Ag6345 Highest expression of this gene was detected in adipose and skeletal muscle (CTs=31). Significant expression of this gene was also seen in pancreas, and kidney.

Panel 2D Summary: Ag2840 This gene was moderately expressed in all tissue samples this panel, with highest expression in metastatic breast cancer (CT=24.5). This gene showed higher expression in normal kidney, prostate, colon and bladder samples relative to the corresponding adjacent tumor sample. Expression of this gene is useful to differentiate between normal and cancerous tissues. Therapeutic modulation of this gene, expressed protein and/or use of agonists targeting this gene or gene product is useful in the treatment of kidney, prostate, colon and bladder cancers.

Panel 3D Summary: Ag2840 This gene showed low levels of expression in the cancer cell lines, with highest expression in the gastric cancer MKN-45 cell line (CT=32). Significant expression was seen in cancer cell lines derived from lung cancer, pancreatic cancer and a leukemia. Therapeutic modulation of this gene, expressed protein and/or use of antibodies or small molecule drug targeting this gene or gene product is useful in the treatment of these cancers.

Panel 4.1D Summary: Ag2840 This transcript was expressed in colon and in resting neutrophils (CTs=31-33). The transcript or the protein it encodes are isefi; as markers to detect colon tissue and neutrophils. Therapeutics modulation of this gene, expressed protein and/or use of antibodies or small molecule targeting this gene or gene product is useful in the treatment of autoimmune and inflammatory diseases such as asthma, allergies, inflammatory bowel disease including Crohn's and Colitis, lupus erythematosus, psoriasis, rheumatoid arthritis, and osteoarthritis.

Panel 5D Summary: Ag2840 This gene was moderately expressed in clinical specimens of adipose, skeletal muscle and uterus. This confirms expression of this gene in tissues with metabolic function. Please see Panel 1.5D for discussion of utility of this gene in metabolic disease.

AL. CG5590801: Integrin A and FG GAP Domain Protein

Expression of full-length physical clone CG55908-01 was assessed using the primer-probe sets Ag6400, Ag6446, Ag6449 and Ag6964, described in Tables ALA, ALB, ALC and ALD. Results of the RTQ-PCR runs are shown in Table ALE.

TABLE ALA
Probe Name Ag6400

| | | | | |
|---|---|---|---|---|
| | | | Start | SEQ ID |
| Primers | Sequences | Length | Position | No |

| | | | | |
|---|---|---|---|---|
| Forward | 5′-gagctgatatgcaaaaggaaag-3′ | 22 | 300 | 1459 |
| Probe | TET-5′-ctgactcccaaccactggttctcctt-3′- | 26 | 323 | 1460 |
| | TAMRA | | | |
| Reverse | 5′-gggcgcagtcaacaatct-3′ | 18 | 375 | 1461 |

TABLE ALB
Probe Name Ag6446

| | | | | |
|---|---|---|---|---|
| | | | Start | SEQ ID |
| Primers | Sequences | Length | Position | No |

| | | | | |
|---|---|---|---|---|
| Forward | 5′-gcttcttccatcggagca-3′ | 18 | 726 | 1462 |
| Probe | TET-5′-caactatcaccgggcctgtctggc-3′- | 24 | 766 | 1463 |
| | TAMRA | | | |
| Reverse | 5′-catggctgaaggctgca-3′ | 17 | 792 | 1464 |

TABLE ALC
Probe Name Ag6449

| | | | | |
|---|---|---|---|---|
| | | | Start | SEQ ID |
| Primers | Sequences | Length | Position | No |

| | | | | |
|---|---|---|---|---|
| Forward | 5′-gagtcagtgttcggagcca-3′ | 19 | 342 | 1465 |
| Probe | TET-5′-ctgcccactctacagctttgaccgc-3′- | 25 | 421 | 1466 |
| | TAMRA | | | |
| Reverse | 5′-cccagacatgcagcacag-3′ | 18 | 450 | 1467 |

TABLE ALD
Probe Name Ag6964

| | | | | |
|---|---|---|---|---|
| | | | Start | SEQ ID |
| Primers | Sequences | Length | Position | No |

| | | | | |
|---|---|---|---|---|
| Forward | 5′-ggccccagacatgca-3′ | 15 | 456 | 1468 |
| Probe | TET-5′-actctacagctttgacccgcgcgg-3′-TAMRA | 23 | 427 | 1469 |
| Reverse | 5′-gccaactgtgtggtgttca-3′ | 19 | 401 | 1470 |

TABLE ALE
General_screening_panel_v1.6

| Tissue Name | A | B | C | D |
|---|---|---|---|---|
| Adipose | 0.0 | 1.7 | 0.0 | 18.8 |
| Melanoma* Hs688(A).T | 0.0 | 0.1 | 0.0 | 0.7 |
| Melanoma* Hs688(B).T | 0.0 | 0.1 | 0.0 | 2.4 |
| Melanoma* M14 | 0.0 | 0.1 | 0.0 | 0.7 |
| Melanoma* LOXIMVI | 0.0 | 0.1 | 0.0 | 0.1 |
| Melanoma* SK-MEL-5 | 0.0 | 6.8 | 0.0 | 15.9 |
| Squamous cell carcinoma SCC-4 | 0.0 | 0.0 | 0.0 | 0.1 |
| Testis Pool | 0.0 | 5.8 | 0.0 | 9.9 |
| Prostate ca.* (bone met) PC-3 | 0.0 | 7.7 | 0.0 | 4.3 |
| Prostate Pool | 0.0 | 1.9 | 0.0 | 10.0 |
| Placenta | 0.0 | 0.9 | 0.0 | 0.4 |
| Uterus Pool | 0.0 | 0.3 | 0.0 | 4.1 |
| Ovarian ca. OVCAR-3 | 0.0 | 4.8 | 0.0 | 4.0 |
| Ovarian ca. SK-OV-3 | 0.0 | 2.5 | 0.0 | 1.7 |
| Ovarian ca. OVCAR-4 | 0.0 | 0.5 | 0.0 | 0.5 |
| Ovarian ca. OVCAR-5 | 0.0 | 15.6 | 0.0 | 7.9 |
| Ovarian ca. IGROV-1 | 22.2 | 5.4 | 79.0 | 75.8 |
| Ovarian ca. OVCAR-8 | 0.0 | 4.2 | 0.0 | 16.7 |
| Ovary | 0.0 | 0.2 | 0.0 | 2.4 |
| Breast ca. MCF-7 | 0.0 | 0.9 | 0.0 | 0.5 |
| Breast ca. MDA-MB-231 | 0.0 | 0.2 | 0.0 | 0.3 |
| Breast ca. BT 549 | 0.0 | 0.2 | 0.0 | 0.4 |
| Breast ca. T47D | 0.0 | 0.7 | 0.0 | 0.5 |
| Breast ca. MDA-N | 0.0 | 0.0 | 10.2 | 0.8 |
| Breast Pool | 0.0 | 2.0 | 0.0 | 16.7 |
| Trachea | 0.0 | 0.5 | 0.0 | 5.6 |
| Lung | 0.0 | 0.5 | 0.0 | 5.1 |
| Fetal Lung | 0.0 | 0.5 | 0.0 | 6.1 |
| Lung ca. NCI-N417 | 0.0 | 0.4 | 0.0 | 2.3 |
| Lung ca. LX-1 | 0.0 | 100.0 | 0.0 | 44.1 |
| Lung ca. NCI-H146 | 0.0 | 0.1 | 0.0 | 0.1 |
| Lung ca. SHP-77 | 0.0 | 0.1 | 0.0 | 3.8 |
| Lung ca. A549 | 0.0 | 14.3 | 0.0 | 4.7 |
| Lung ca. NCI-H526 | 0.0 | 0.0 | 0.0 | 0.5 |
| Lung ca. NCI-H23 | 0.0 | 15.9 | 0.0 | 10.3 |
| Lung ca. NCI-H460 | 0.0 | 0.1 | 0.0 | 0.3 |
| Lung ca. HOP-62 | 0.0 | 0.2 | 0.0 | 0.7 |
| Lung ca. NCI-H522 | 0.0 | 27.7 | 0.0 | 8.9 |
| Liver | 0.0 | 5.3 | 0.0 | 2.0 |
| Fetal Liver | 0.0 | 23.0 | 0.0 | 8.2 |
| Liver ca. HepG2 | 0.0 | 7.3 | 0.0 | 2.4 |
| Kidney Pool | 21.9 | 5.3 | 0.0 | 32.8 |
| Fetal Kidney | 0.0 | 20.2 | 0.0 | 11.5 |
| Renal ca. 786-0 | 0.0 | 1.7 | 0.0 | 0.9 |
| Renal ca. A498 | 0.0 | 23.0 | 0.0 | 8.5 |
| Renal ca. ACHN | 0.0 | 3.8 | 0.0 | 2.5 |
| Renal ca. UO-31 | 0.0 | 0.7 | 0.0 | 0.3 |
| Renal ca. TK-10 | 0.0 | 6.4 | 0.0 | 4.6 |
| Bladder | 0.0 | 3.2 | 0.0 | 6.7 |
| Gastric ca. (liver met.) NCI-N87 | 0.0 | 17.8 | 0.0 | 6.7 |
| Gastric ca. KATO III | 0.0 | 1.3 | 0.0 | 0.9 |
| Colon ca. SW-948 | 0.0 | 6.1 | 0.0 | 1.2 |
| Colon ca. SW480 | 21.6 | 39.0 | 0.0 | 33.7 |
| Colon ca.* (SW480 met) SW620 | 0.0 | 71.2 | 0.0 | 25.0 |
| Colon ca. HT29 | 0.0 | 3.5 | 0.0 | 0.3 |
| Colon ca. HCT-116 | 0.0 | 6.4 | 0.0 | 4.3 |
| Colon ca. CaCo-2 | 12.2 | 78.5 | 0.0 | 38.2 |
| Colon cancer tissue | 0.0 | 21.9 | 0.0 | 20.4 |
| Colon ca. SW1116 | 0.0 | 19.5 | 0.0 | 6.0 |
| Colon ca. Colo-205 | 0.0 | 3.0 | 0.0 | 0.8 |
| Colon ca. SW-48 | 0.0 | 4.2 | 0.0 | 2.6 |
| Colon Pool | 0.0 | 3.1 | 5.1 | 20.6 |
| Small Intestine Pool | 0.0 | 2.5 | 0.0 | 10.4 |
| Stomach Pool | 0.0 | 1.1 | 0.0 | 10.7 |
| Bone Marrow Pool | 0.0 | 1.1 | 0.0 | 12.5 |
| Fetal Heart | 0.0 | 2.7 | 0.0 | 20.7 |
| Heart Pool | 0.0 | 3.4 | 0.0 | 26.1 |
| Lymph Node Pool | 0.0 | 2.8 | 0.0 | 24.7 |
| Fetal Skeletal Muscle | 0.0 | 57.0 | 0.0 | 50.7 |
| Skeletal Muscle Pool | 0.0 | 24.3 | 0.0 | 32.3 |
| Spleen Pool | 0.0 | 2.6 | 0.0 | 3.1 |
| Thymus Pool | 0.0 | 1.4 | 0.0 | 7.0 |
| CNS cancer (glio/astro) U87-MG | 0.0 | 6.3 | 0.0 | 14.1 |
| CNS cancer (glio/astro) U-118-MG | 0.0 | 5.1 | 0.0 | 5.8 |
| CNS cancer (neuro; met) SK-N-AS | 0.0 | 3.9 | 0.0 | 2.6 |
| CNS cancer (astro) SF-539 | 0.0 | 0.3 | 0.0 | 0.1 |
| CNS cancer (astro) SNB-75 | 0.0 | 2.4 | 0.0 | 9.7 |
| CNS cancer (glio) SNB-19 | 0.0 | 5.2 | 100.0 | 100.0 |
| CNS cancer (glio) SF-295 | 0.0 | 14.9 | 0.0 | 14.8 |
| Brain (Amygdala) Pool | 0.0 | 1.1 | 0.0 | 5.3 |
| Brain (cerebellum) | 0.0 | 1.4 | 0.0 | 9.7 |
| Brain (fetal) | 0.0 | 1.1 | 0.0 | 6.4 |
| Brain (Hippocampus) Pool | 0.0 | 2.0 | 0.0 | 10.2 |
| Cerebral Cortex Pool | 0.0 | 2.0 | 0.0 | 8.7 |
| Brain (Substantia nigra) Pool | 0.0 | 1.1 | 0.0 | 9.3 |
| Brain (Thalamus) Pool | 0.0 | 3.2 | 0.0 | 8.7 |
| Brain (whole) | 0.0 | 1.9 | 0.0 | 8.7 |
| Spinal Cord Pool | 100.0 | 2.9 | 0.0 | 9.0 |
| Adrenal Gland | 0.0 | 0.7 | 0.0 | 4.1 |
| Pituitary gland Pool | 0.0 | 0.4 | 0.0 | 0.5 |
| Salivary Gland | 0.0 | 0.2 | 0.0 | 1.0 |
| Thyroid (female) | 0.0 | 0.8 | 0.0 | 2.3 |
| Pancreatic ca. CAPAN2 | 0.0 | 4.6 | 0.0 | 2.2 |
| Pancreas Pool | 0.0 | 2.6 | 0.0 | 2.3 |

Column A - Rel. Exp. (%) Ag6400, Run 277248363
Column B - Rel. Exp. (%) Ag6446, Run 277250179
Column C - Rel. Exp. (%) Ag6449, Run 277250185
Column D - Rel. Exp. (%) Ag6964, Run 278388946

General_screening_panel_v1.6 Summary: Highest expression of this gene was detected in an ovarian cancer IGROV-1 cell line and a brain cancer SNB-19 cell line (CTs=25-33.7). Moderate to low levels of expression of this gene are also seen in all the regions of central nervous system, tissues with metabolic/endocrine functions, and number of cancer cell lines.

AM. CG56110-03: B7 H1

Expression of full-length physical clone CG56110-03 was assessed using the primer-probe set Ag1544, described in Table AMA. Results of the RTQ-PCR runs are shown in Tables AMB, AMC and AMD.

TABLE AMA
Probe Name Ag1544

| | | | | |
|---|---|---|---|---|
| | | | | SEQ |
| | | | Start | ID |
| Primers | Sequences | Length | Position | No |

| | | | | |
|---|---|---|---|---|
| Forward | 5′-tctggacaagcagtgaccat-3′ | 20 | 497 | 1471 |
| Probe | TET-5′-accaccaccaattccaagagagagga-3′- | 26 | 538 | 1472 |
| | TAMRA | | | |
| Reverse | 5′-ttctcagtgtgctggtcaca-3′ | 120 | 576 | 1473 |

TABLE AMB
Panel 1.3D

| Tissue Name | A |
|---|---|
| Liver adenocarcinoma | 3.6 |
| Pancreas | 2.9 |
| Pancreatic ca. CAPAN 2 | 5.0 |
| Adrenal gland | 2.4 |
| Thyroid | 2.3 |
| Salivary gland | 1.7 |
| Pituitary gland | 6.3 |
| Brain (fetal) | 1.8 |
| Brain (whole) | 3.5 |
| Brain (amygdala) | 2.8 |
| Brain (cerebellum) | 1.7 |
| Brain (hippocampus) | 4.5 |
| Brain (substantia nigra) | 2.7 |
| Brain (thalamus) | 4.4 |
| Cerebral Cortex | 10.4 |
| Spinal cord | 2.0 |
| glio/astro U87-MG | 25.5 |
| glio/astro U-118-MG | 8.2 |
| astrocytoma SW1783 | 7.9 |
| neuro*; met SK-N-AS | 9.2 |
| astrocytoma SF-539 | 6.3 |
| astrocytoma SNB-75 | 37.9 |
| glioma SNB-19 | 12.3 |
| glioma U251 | 14.7 |
| glioma SF-295 | 6.0 |
| Heart (fetal) | 9.0 |
| Heart | 5.4 |
| Skeletal muscle (fetal) | 6.2 |
| Skeletal muscle | 1.9 |
| Bone marrow | 3.2 |
| Thymus | 13.4 |
| Spleen | 14.0 |
| Lymph node | 27.0 |
| Colorectal | 1.5 |
| Stomach | 3.4 |
| Small intestine | 2.7 |
| Colon ca. SW480 | 2.2 |
| Colon ca.* SW620(SW480 met) | 0.8 |
| Colon ca. HT29 | 1.6 |
| Colon ca. HCT-116 | 2.6 |
| Colon ca. CaCo-2 | 0.6 |
| Colon ca. tissue(ODO3866) | 6.6 |
| Colon ca. HCC-2998 | 4.5 |
| Gastric ca.* (liver met) NCI-N87 | 100.0 |
| Bladder | 6.7 |
| Trachea | 10.9 |
| Kidney | 0.8 |
| Kidney (fetal) | 5.5 |
| Renal ca. 786-0 | 10.0 |
| Renal ca. A498 | 17.8 |
| Renal ca. RXF 393 | 11.0 |
| Renal ca. ACHN | 6.9 |
| Renal ca. UO-31 | 14.4 |
| Renal ca. TK-10 | 0.2 |
| Liver | 2.2 |
| Liver (fetal) | 5.8 |
| Liver ca. (hepatoblast) HepG2 | 0.1 |
| Lung | 27.9 |
| Lung (fetal) | 14.0 |
| Lung ca. (small cell) LX-1 | 3.4 |
| Lung ca. (small cell) NCI-H69 | 0.2 |
| Lung ca. (s. cell var.) SHP-77 | 3.0 |
| Lung ca. (large cell)NCI-H460 | 9.7 |
| Lung ca. (non-sm. cell) A549 | 0.8 |
| Lung ca. (non-s. cell) NCI-H23 | 1.3 |
| Lung ca. (non-s. cell) HOP-62 | 19.3 |
| Lung ca. (non-s. cl) NCI-H522 | 0.0 |
| Lung ca. (squam.) SW 900 | 33.2 |
| Lung ca. (squam.) NCI-H596 | 0.0 |
| Mammary gland | 5.6 |
| Breast ca.* (pl. ef) MCF-7 | 0.0 |
| Breast ca.* (pl. ef) MDA-MB-231 | 62.0 |
| Breast ca.* (pl. ef) T47D | 0.0 |
| Breast ca. BT-549 | 11.1 |
| Breast ca. MDA-N | 5.9 |
| Ovary | 3.5 |
| Ovarian ca. OVCAR-3 | 1.7 |
| Ovarian ca. OVCAR-4 | 0.1 |
| Ovarian ca. OVCAR-5 | 5.9 |
| Ovarian ca. OVCAR-8 | 3.6 |
| Ovarian ca. IGROV-1 | 0.0 |
| Ovarian ca.* (ascites) SK-OV-3 | 0.6 |
| Uterus | 2.7 |
| Placenta | 59.0 |
| Prostate | 1.7 |
| Prostate ca.* (bone met)PC-3 | 3.1 |
| Testis | 3.7 |
| Melanoma Hs688(A).T | 15.1 |
| Melanoma* (met) Hs688(B).T | 9.7 |
| Melanoma UACC-62 | 1.4 |
| Melanoma M14 | 0.1 |
| Melanoma LOX IMVI | 17.8 |
| Melanoma* (met) SK-MEL-5 | 7.4 |
| Adipose | 5.1 |

Column A - Rel. Exp. (%) Ag1544, Run 146169090

TABLE AMC
Panel 2D

| Tissue Name | A | B |
|---|---|---|
| Normal Colon | 15.7 | 2.5 |
| CC Well to Mod Diff (ODO3866) | 5.1 | 0.8 |
| CC Margin (ODO3866) | 3.8 | 0.5 |
| CC Gr.2 rectosigmoid (ODO3868) | 3.3 | 1.2 |
| CC Margin (ODO3868) | 0.0 | 0.1 |
| CC Mod Diff (ODO3920) | 2.6 | 1.8 |
| CC Margin (ODO3920) | 3.0 | 2.2 |
| CC Gr.2 ascend colon (ODO3921) | 6.7 | 4.0 |
| CC Margin (ODO3921) | 1.6 | 0.9 |
| CC from Partial Hepatectomy | 6.0 | 5.4 |
| (ODO4309) Mets | | |
| Liver Margin (ODO4309) | 9.8 | 4.7 |
| Colon mets to lung (OD04451-01) | 7.3 | 7.2 |
| Lung Margin (OD04451-02) | 13.6 | 2.4 |
| Normal Prostate 6546-1 | 3.5 | 2.2 |
| Prostate Cancer (OD04410) | 5.1 | 3.7 |
| Prostate Margin (OD04410) | 4.5 | 8.0 |
| Prostate Cancer (OD04720-01) | 6.3 | 7.1 |
| Prostate Margin (OD04720-02) | 8.1 | 11.5 |
| Normal Lung 061010 | 34.2 | 49.0 |
| Lung Met to Muscle (ODO4286) | 25.7 | 58.2 |
| Muscle Margin (ODO4286) | 5.7 | 4.9 |
| Lung Malignant Cancer (OD03126) | 13.9 | 25.7 |
| Lung Margin (OD03126) | 39.0 | 32.3 |
| Lung Cancer (OD04404) | 27.4 | 15.1 |
| Lung Margin (OD04404) | 25.0 | 4.9 |
| Lung Cancer (OD04565) | 6.2 | 5.8 |
| Lung Margin (OD04565) | 12.9 | 17.1 |
| Lung Cancer (OD04237-01) | 27.5 | 17.6 |
| Lung Margin (OD04237-02) | 100.0 | 28.9 |
| Ocular Mel Met to Liver (ODO4310) | 3.3 | 3.9 |
| Liver Margin (ODO4310) | 5.0 | 5.6 |
| Melanoma Mets to Lung (OD04321) | 5.5 | 3.7 |
| Lung Margin (OD04321) | 35.8 | 32.1 |
| Normal Kidney | 12.1 | 4.8 |
| Kidney Ca, Nuclear grade 2 (OD04338) | 12.7 | 11.9 |
| Kidney Margin (OD04338) | 6.7 | 2.6 |
| Kidney Ca Nuclear grade 1/2 (OD04339) | 14.7 | 9.2 |
| Kidney Margin (OD04339) | 6.6 | 3.2 |
| Kidney Ca, Clear cell type (OD04340) | 7.3 | 9.4 |
| Kidney Margin (OD04340) | 8.2 | 5.1 |
| Kidney Ca, Nuclear grade 3 (OD04348) | 6.7 | 8.1 |
| Kidney Margin (OD04348) | 17.9 | 18.9 |
| Kidney Cancer (OD04622-01) | 7.5 | 15.9 |
| Kidney Margin (OD04622-03) | 1.9 | 0.7 |
| Kidney Cancer (OD04450-01) | 1.7 | 2.3 |
| Kidney Margin (OD04450-03) | 6.5 | 5.9 |
| Kidney Cancer 8120607 | 4.5 | 2.5 |
| Kidney Margin 8120608 | 0.7 | 0.8 |
| Kidney Cancer 8120613 | 1.2 | 1.7 |
| Kidney Margin 8120614 | 0.0 | 0.3 |
| Kidney Cancer 9010320 | 4.7 | 1.2 |
| Kidney Margin 9010321 | 2.1 | 0.7 |
| Normal Uterus | 2.7 | 1.6 |
| Uterus Cancer 064011 | 6.0 | 6.3 |
| Normal Thyroid | 7.3 | 4.5 |
| Thyroid Cancer 064010 | 36.3 | 29.9 |
| Thyroid Cancer A302152 | 11.6 | 13.8 |
| Thyroid Margin A302153 | 15.7 | 9.9 |
| Normal Breast | 5.2 | 3.9 |
| Breast Cancer (OD04566) | 5.0 | 3.3 |
| Breast Cancer (OD04590-01) | 8.6 | 2.3 |
| Breast Cancer Mets (OD04590-03) | 6.7 | 7.2 |
| Breast Cancer Metastasis (OD04655-05) | 6.6 | 5.6 |
| Breast Cancer 064006 | 15.3 | 11.2 |
| Breast Cancer 1024 | 1.9 | 0.9 |
| Breast Cancer 9100266 | 3.1 | 3.5 |
| Breast Margin 9100265 | 2.4 | 1.9 |
| Breast Cancer A209073 | 5.1 | 7.2 |
| Breast Margin A209073 | 5.4 | 1.2 |
| Normal Liver | 6.0 | 4.3 |
| Liver Cancer 064003 | 4.7 | 1.1 |
| Liver Cancer 1025 | 1.8 | 1.5 |
| Liver Cancer 1026 | 0.0 | 0.5 |
| Liver Cancer 6004-T | 4.3 | 1.1 |
| Liver Tissue 6004-N | 4.2 | 1.3 |
| Liver Cancer 6005-T | 0.5 | 0.4 |
| Liver Tissue 6005-N | 1.6 | 1.0 |
| Normal Bladder | 11.3 | 12.7 |
| Bladder Cancer 1023 | 1.8 | 1.3 |
| Bladder Cancer A302173 | 50.0 | 16.7 |
| Bladder Cancer (OD04718-01) | 96.6 | 100.0 |
| Bladder Normal Adjacent (OD04718-03) | 11.1 | 4.6 |
| Normal Ovary | 0.3 | 0.4 |
| Ovarian Cancer 064008 | 7.4 | 3.4 |
| Ovarian Cancer (OD04768-07) | 23.0 | 0.4 |
| Ovary Margin (OD04768-08) | 5.5 | 2.0 |
| Normal Stomach | 5.2 | 2.1 |
| Gastric Cancer 9060358 | 1.8 | 0.4 |
| Stomach Margin 9060359 | 2.3 | 1.3 |
| Gastric Cancer 9060395 | 5.9 | 2.0 |
| Stomach Margin 9060394 | 5.0 | 1.9 |
| Gastric Cancer 9060397 | 6.0 | 2.6 |
| Stomach Margin 9060396 | 2.1 | 1.5 |
| Gastric Cancer 064005 | 10.9 | 10.4 |

Column A - Rel. Exp. (%) Ag1544, Run 145030196
Column B - Rel. Exp. (%) Ag1544, Run 145361330

TABLE AMD
Panel 4.1D

| Tissue Name | A |
|---|---|
| Secondary Th1 act | 7.9 |
| Secondary Th2 act | 11.7 |
| Secondary Tr1 act | 7.3 |
| Secondary Th1 rest | 0.8 |
| Secondary Th2 rest | 1.2 |
| Secondary Tr1 rest | 1.1 |
| Primary Th1 act | 6.7 |
| Primary Th2 act | 4.5 |
| Primary Tr1 act | 5.9 |
| Primary Th1 rest | 0.4 |
| Primary Th2 rest | 0.3 |
| Primary Tr1 rest | 0.3 |
| CD45RA CD4 lymphocyte act | 5.8 |
| CD45RO CD4 lymphocyte act | 6.5 |
| CD8 lymphocyte act | 2.5 |
| Secondary CD8 lymphocyte rest | 5.9 |
| Secondary CD8 lymphocyte act | 0.7 |
| CD4 lymphocyte none | 0.4 |
| 2ry Th1/Th2/Tr1_anti-CD95 CH11 | 1.7 |
| LAK cells rest | 23.8 |
| LAK cells IL-2 | 2.0 |
| LAK cells IL-2 + IL-12 | 3.1 |
| LAK cells IL-2 + IFN gamma | 5.6 |
| LAK cells IL-2 + IL-18 | 6.6 |
| LAK cells PMA/ionomycin | 77.9 |
| NK Cells IL-2 rest | 1.5 |
| Two Way MLR 3 day | 21.6 |
| Two Way MLR 5 day | 18.9 |
| Two Way MLR 7 day | 5.1 |
| PBMC rest | 0.3 |
| PBMC PWM | 11.5 |
| PBMC PHA-L | 8.4 |
| Ramos (B cell) none | 0.2 |
| Ramos (B cell) ionomycin | 0.3 |
| B lymphocytes PWM | 2.8 |
| B lymphocytes CD40L and IL-4 | 1.2 |
| EOL-1 dbcAMP | 0.1 |
| EOL-1 dbcAMP PMA/ionomycin | 0.2 |
| Dendritic cells none | 19.9 |
| Dendritic cells LPS | 58.2 |
| Dendritic cells anti-CD40 | 12.6 |
| Monocytes rest | 0.1 |
| Monocytes LPS | 100.0 |
| Macrophages rest | 4.6 |
| Macrophages LPS | 32.3 |
| HUVEC none | 2.0 |
| HUVEC starved | 3.2 |
| HUVEC IL-1beta | 4.0 |
| HUVEC IFN gamma | 18.4 |
| HUVEC TNF alpha + IFN gamma | 19.5 |
| HUVEC TNF alpha + IL4 | 2.8 |
| HUVEC IL-11 | 2.0 |
| Lung Microvascular EC none | 7.5 |
| Lung Microvascular EC TNFalpha + IL-1beta | 3.5 |
| Microvascular Dermal EC none | 5.2 |
| Microsvasular Dermal EC TNFalpha + IL-1beta | 1.4 |
| Bronchial epithelium TNFalpha + IL1beta | 1.1 |
| Small airway epithelium none | 0.7 |
| Small airway epithelium TNFalpha + IL-1beta | 2.3 |
| Coronery artery SMC rest | 1.4 |
| Coronery artery SMC TNFalpha + IL-1beta | 1.8 |
| Astrocytes rest | 3.3 |
| Astrocytes TNFalpha + IL-1beta | 5.8 |
| KU-812 (Basophil) rest | 0.0 |
| KU-812 (Basophil) PMA/ionomycin | 0.3 |
| CCD1106 (Keratinocytes) none | 5.4 |
| CCD1106 (Keratinocytes) TNFalpha + IL-1beta | 19.9 |
| Liver cirrhosis | 0.2 |
| NCI-H292 none | 0.8 |
| NCI-H292 IL-4 | 1.4 |
| NCI-H292 IL-9 | 1.0 |
| NCI-H292 IL-13 | 1.6 |
| NCI-H292 IFN gamma | 4.2 |
| HPAEC none | 2.4 |
| HPAEC TNF alpha + IL-1 beta | 4.7 |
| Lung fibroblast none | 0.8 |
| Lung fibroblast TNF alpha + IL-1 beta | 1.6 |
| Lung fibroblast IL-4 | 1.5 |
| Lung fibroblast IL-9 | 1.7 |
| Lung fibroblast IL-13 | 1.1 |
| Lung fibroblast IFN gamma | 15.0 |
| Dermal fibroblast CCD1070 rest | 2.8 |
| Dermal fibroblast CCD1070 TNF alpha | 4.9 |
| Dermal fibroblast CCD1070 IL-1 beta | 4.0 |
| Dermal fibroblast IFN gamma | 3.7 |
| Dermal fibroblast IL-4 | 0.7 |
| Dermal Fibroblasts rest | 0.2 |
| Neutrophils TNFa + LPS | 0.4 |
| Neutrophils rest | 0.6 |
| Colon | 0.2 |
| Lung | 0.6 |
| Thymus | 4.5 |
| Kidney | 0.5 |

Column A - Rel. Exp. (%) Ag1544, Run 209988812

Panel 1.3D Summary: Ag1544 The expression of this gene was highest in a sample derived from a gastric cancer cell line (NCI-H87) (CT=27.3). There was substantial expression found in lung cancer cell lines, a breast cancer cell line and placental tissue. The expression of this gene is useful as a marker to distinguish NCI-H87 cells from other samples in the panel. Therapeutic modulation of this gene or its protein product, through the use of small molecule drugs, antibodies or protein therapeutics is beneficial in the treatment of lung cancer or breast cancer.

Panel 2D Summary: Ag1544 The expression of this gene was highest expression associated with bladder cancer tissue and lung tissue derived samples. The expression of this gene is useful as a marker to distinguish between these samples and the rest of the samples in the panel. Therapeutic modulation of this gene, through the use of small molecule drugs, antibodies or protein therapeutics are beneficial in the treatment of bladder or lung cancer.

Panel 4.1D Summary: Ag1544 This transcript was expressed in LAK cells, and induced in LAK cells activated with PMA/ionomycin, dendritic cells treated with LPS, monocytes treated with LPS, Gamma interferon treated HUEC cells and keratinocytes treated with TNFalpha and IL-1beta. This transcript encodes a smaller isoform of B7-H1, an antigen presentation co-receptor. B7-H1 binds to PD-1 ligand on T cells, resulting in T cell activation and production of IL-10. Antibody or other types of therapeutics designed with B7-H1 are useful for blocking T cell activation and for the treatment of T cell-mediated diseases such as asthma, psoriasis, IBD and arthritis. Alternatively, agonistic therapeutics designed with this protein are useful for adjuvant or immuno-modulatory treatments.

AN. CG56110-07: B7-H1

Expression of full-length physical clone CG56110-07 was assessed using the primer-probe set Ag5282, described in Table ANA. Results of the RTQ-PCR runs are shown in Tables ANB and ANC.

TABLE ANA
Probe Name Ag5282

| | | | | SEQ |
|---|---|---|---|---|
| | | | Start | ID |
| Primers | Sequences | Length | Position | No |
| Forward | 5′-tcaagtcctgagtggagattagat-3′ | 24 | 491 | 1474 |
| Probe | TET-5′-tggtcatcccagaactacctctggca-3′- | 26 | 540 | 1475 |
| | TAMRA | | | |
| Reverse | 5′-cccagaattaccaagtgagtcct-3′ | 23 | 581 | 1476 |

TABLE ANB
General_screening_panel_v1.5

| Tissue Name | A |
|---|---|
| Adipose | 0.0 |
| Melanoma* Hs688(A).T | 0.0 |
| Melanoma* Hs688(B).T | 5.1 |
| Melanoma* M14 | 0.6 |
| Melanoma* LOXIMVI | 23.0 |
| Melanoma* SK-MEL-5 | 3.6 |
| Squamous cell carcinoma SCC-4 | 10.9 |
| Testis Pool | 0.0 |
| Prostate ca.* (bone met) PC-3 | 0.0 |
| Prostate Pool | 1.2 |
| Placenta | 2.8 |
| Uterus Pool | 2.8 |
| Ovarian ca. OVCAR-3 | 2.7 |
| Ovarian ca. SK-OV-3 | 0.0 |
| Ovarian ca. OVCAR-4 | 0.0 |
| Ovarian ca. OVCAR-5 | 6.1 |
| Ovarian ca. IGROV-1 | 5.4 |
| Ovarian ca. OVCAR-8 | 1.3 |
| Ovary | 0.0 |
| Breast ca. MCF-7 | 0.0 |
| Breast ca. MDA-MB-231 | 31.2 |
| Breast ca. BT 549 | 3.6 |
| Breast ca. T47D | 0.0 |
| Breast ca. MDA-N | 3.7 |
| Breast Pool | 0.5 |
| Trachea | 1.1 |
| Lung | 1.2 |
| Fetal Lung | 1.3 |
| Lung ca. NCI-N417 | 0.0 |
| Lung ca. LX-1 | 2.7 |
| Lung ca. NCI-H146 | 0.0 |
| Lung ca. SHP-77 | 0.0 |
| Lung ca. A549 | 0.0 |
| Lung ca. NCI-H526 | 0.0 |
| Lung ca. NCI-H23 | 0.8 |
| Lung ca. NCI-H460 | 1.4 |
| Lung ca. HOP-62 | 13.1 |
| Lung ca. NCI-H522 | 0.0 |
| Liver | 0.0 |
| Fetal Liver | 0.0 |
| Liver ca. HepG2 | 0.0 |
| Kidney Pool | 3.1 |
| Fetal Kidney | 0.0 |
| Renal ca. 786-0 | 5.1 |
| Renal ca. A498 | 2.1 |
| Renal ca. ACHN | 1.3 |
| Renal ca. UO-31 | 6.8 |
| Renal ca. TK-10 | 0.7 |
| Bladder | 4.0 |
| Gastric ca. (liver met.) NCI-N87 | 100.0 |
| Gastric ca. KATO III | 4.2 |
| Colon ca. SW-948 | 0.0 |
| Colon ca. SW480 | 2.2 |
| Colon ca.* (SW480 met) SW620 | 0.0 |
| Colon ca. HT29 | 0.0 |
| Colon ca. HCT-116 | 3.6 |
| Colon ca. CaCo-2 | 0.0 |
| Colon cancer tissue | 1.8 |
| Colon ca. SW1116 | 0.0 |
| Colon ca. Colo-205 | 0.0 |
| Colon ca. SW-48 | 0.0 |
| Colon Pool | 4.8 |
| Small Intestine Pool | 4.3 |
| Stomach Pool | 0.0 |
| Bone Marrow Pool | 0.9 |
| Fetal Heart | 3.9 |
| Heart Pool | 2.3 |
| Lymph Node Pool | 0.0 |
| Fetal Skeletal Muscle | 1.2 |
| Skeletal Muscle Pool | 3.1 |
| Spleen Pool | 3.9 |
| Thymus Pool | 3.9 |
| CNS cancer (glio/astro) U87-MG | 21.9 |
| CNS cancer (glio/astro) U-118-MG | 3.5 |
| CNS cancer (neuro; met) SK-N-AS | 1.4 |
| CNS cancer (astro) SF-539 | 0.0 |
| CNS cancer (astro) SNB-75 | 2.5 |
| CNS cancer (glio) SNB-19 | 8.7 |
| CNS cancer (glio) SF-295 | 3.6 |
| Brain (Amygdala) Pool | 0.0 |
| Brain (cerebellum) | 1.3 |
| Brain (fetal) | 0.0 |
| Brain (Hippocampus) Pool | 0.0 |
| Cerebral Cortex Pool | 2.1 |
| Brain (Substantia nigra) Pool | 0.0 |
| Brain (Thalamus) Pool | 0.9 |
| Brain (whole) | 0.0 |
| Spinal Cord Pool | 1.4 |
| Adrenal Gland | 2.2 |
| Pituitary gland Pool | 0.0 |
| Salivary Gland | 0.0 |
| Thyroid (female) | 2.5 |
| Pancreatic ca. CAPAN2 | 12.3 |
| Pancreas Pool | 1.8 |

Column A - Rel. Exp. (%) Ag5282, Run 230565189

TABLE ANC
Panel 4.1D

| Tissue Name | A |
|---|---|
| Secondary Th1 act | 5.6 |
| Secondary Th2 act | 18.6 |
| Secondary Tr1 act | 4.9 |
| Secondary Th1 rest | 0.0 |
| Secondary Th2 rest | 0.3 |
| Secondary Tr1 rest | 0.9 |
| Primary Th1 act | 4.6 |
| Primary Th2 act | 4.1 |
| Primary Tr1 act | 9.7 |
| Primary Th1 rest | 0.0 |
| Primary Th2 rest | 0.8 |
| Primary Tr1 rest | 0.0 |
| CD45RA CD4 lymphocyte act | 9.8 |
| CD45RO CD4 lymphocyte act | 13.1 |
| CD8 lymphocyte act | 0.0 |
| Secondary CD8 lymphocyte rest | 6.7 |
| Secondary CD8 lymphocyte act | 0.8 |
| CD4 lymphocyte none | 0.0 |
| 2ry Th1/Th2/Tr1_anti-CD95 CH11 | 0.0 |
| LAK cells rest | 14.0 |
| LAK cells IL-2 | 0.0 |
| LAK cells IL-2 + IL-12 | 1.9 |
| LAK cells IL-2 + IFN gamma | 1.7 |
| LAK cells IL-2 + IL-18 | 3.0 |
| LAK cells PMA/ionomycin | 76.3 |
| NK Cells IL-2 rest | 1.0 |
| Two Way MLR 3 day | 9.4 |
| Two Way MLR 5 day | 4.0 |
| Two Way MLR 7 day | 0.7 |
| PBMC rest | 0.0 |
| PBMC PWM | 6.1 |
| PBMC PHA-L | 6.1 |
| Ramos (B cell) none | 0.0 |
| Ramos (B cell) ionomycin | 0.0 |
| B lymphocytes PWM | 3.6 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 10.2 |
| Dendritic cells LPS | 36.1 |
| Dendritic cells anti-CD40 | 4.8 |
| Monocytes rest | 0.0 |
| Monocytes LPS | 100.0 |
| Macrophages rest | 2.6 |
| Macrophages LPS | 5.0 |
| HUVEC none | 2.2 |
| HUVEC starved | 3.8 |
| HUVEC IL-1beta | 1.7 |
| HUVEC IFN gamma | 25.5 |
| HUVEC TNF alpha + IFN gamma | 13.9 |
| HUVEC TNF alpha + IL4 | 2.0 |
| HUVEC IL-11 | 0.0 |
| Lung Microvascular EC none | 9.9 |
| Lung Microvascular EC TNFalpha + IL-1beta | 0.0 |
| Microvascular Dermal EC none | 0.8 |
| Microsvasular Dermal EC TNFalpha + IL-1beta | 0.3 |
| Bronchial epithelium TNFalpha + IL1beta | 0.3 |
| Small airway epithelium none | 2.3 |
| Small airway epithelium TNFalpha + IL-1beta | 2.2 |
| Coronery artery SMC rest | 1.0 |
| Coronery artery SMC TNFalpha + IL-1beta | 1.9 |
| Astrocytes rest | 1.8 |
| Astrocytes TNFalpha + IL-1beta | 5.3 |
| KU-812 (Basophil) rest | 0.0 |
| KU-812 (Basophil) PMA/ionomycin | 1.0 |
| CCD1106 (Keratinocytes) none | 5.5 |
| CCD1106 (Keratinocytes) TNFalpha + IL-1beta | 18.8 |
| Liver cirrhosis | 0.0 |
| NCI-H292 none | 0.0 |
| NCI-H292 IL-4 | 1.0 |
| NCI-H292 IL-9 | 1.0 |
| NCI-H292 IL-13 | 1.5 |
| NCI-H292 IFN gamma | 8.8 |
| HPAEC none | 0.9 |
| HPAEC TNF alpha + IL-1 beta | 6.0 |
| Lung fibroblast none | 0.0 |
| Lung fibroblast TNF alpha + IL-1 beta | 1.0 |
| Lung fibroblast IL-4 | 2.3 |
| Lung fibroblast IL-9 | 1.6 |
| Lung fibroblast IL-13 | 0.0 |
| Lung fibroblast IFN gamma | 17.0 |
| Dermal fibroblast CCD1070 rest | 6.6 |
| Dermal fibroblast CCD1070 TNF alpha | 9.4 |
| Dermal fibroblast CCD1070 IL-1 beta | 5.3 |
| Dermal fibroblast IFN gamma | 2.8 |
| Dermal fibroblast IL-4 | 0.0 |
| Dermal Fibroblasts rest | 0.0 |
| Neutrophils TNFa + LPS | 0.9 |
| Neutrophils rest | 0.0 |
| Colon | 0.0 |
| Lung | 0.0 |
| Thymus | 0.0 |
| Kidney | 0.0 |

Column A - Rel. Exp. (%) Ag5282, Run 230510202

General_screening_panel_v1.5 Summary: Ag5282 The expression of this gene was highest in a sample derived from a gastric cancer cell line (NCI-H87) (CT=31).

There was relatively low expression in the remaining samples of panel 1.5. The expression of this gene is useful as a marker to distinguish NCI-H87 cells from other samples in the panel.

In addition, low expression of this gene was also seen in number of cancer cell lines derived from melanoma, pancreatic, gastric, renal, squamous cell carcinoma and lung cancers. Therapeutic modulation of this gene or its protein product is useful in the treatment of these cancers.

Panel 4.1D Summary: Ag5282 This transcript was not expressed in the normal tissue samples on this panel. The transcript was expressed in LAK cells, and induced in LAK cells activated with PMA/ionomycin, dendritic cells treated with LPS, monocytes treated with LPS, Gamma interferon treated HUVEC cells and keratinocytes treated with TNFalpha and IL-1beta. This transcript encodes a smaller isoform of B7-H1, an antigen presentation co-receptor. B7-H1 binds to PD-1 ligand on T cells and resulting in T cell activation and production of IL-10. Antibody or other types of therapeutics designed with B7-H1 are useful for blocking T cell activation and in the treatment of T cell-mediated diseases such as asthma, psoriasis, IBD and arthritis. Alternatively, agonistic therapeutics designed with this protein are useful for adjuvant like properties.

AO. CG56449-09: MEGF6/NOTCH like Isoform 7

Expression of gene CG56449-09 was assessed using the primer-probe sets Ag1153, Ag1937, Ag422 and Ag252 and, described in Tables AOA, AOB, AOC, and AOD. Results of the RTQ-PCR runs are shown in Tables AOE, AOF, and AOG.

TABLE AOA
Probe Name Ag1513

| | | | | SEQ |
|---|---|---|---|---|
| | | | Start | ID |
| Primers | Sequences | Length | Position | No |
| Forward | 5′-acacacgctcacagagaagttt-3′ | 22 | 1521 | 1477 |
| Probe | TET-5′-ctggatgactcctttggccatgact-3′- | 25 | 1549 | 1478 |
| | TAMRA | | | |
| Reverse | 5′-ctgcagtcatcacaggtcaag-3′ | 21 | 1578 | 1479 |

TABLE AOB
Probe Name Ag1937

| | | | | SEQ |
|---|---|---|---|---|
| | | | Start | ID |
| Primers | Sequences | Length | Position | No |
| Forward | 5′-ctgcagtcatcacaggtcaag-3′ | 21 | 1578 | 1480 |
| Probe | TET-5′-ccaaaggagtcatccaggcagacaaa-3′- | 26 | 1540 | 1481 |
| | TAMRA | | | |
| Reverse | 5′-gaacacacgctcacagagaag-3′ | 21 | 1519 1482 | |

TABLE AOC
Probe Name Ag422

| | | | | SEQ |
|---|---|---|---|---|
| | | | Start | ID |
| Primers | Sequences | Length | Position | No |
| Forward | 5′-tgaacaccccaggctcctac-3′ | 20 | 545 | 1483 |
| Probe | TET-5′-cggcttccggctccacactgac-3′- | 22 | 582 | 1484 |
| | TAMRA | | | |
| Reverse | 5′-taatggccaggcaggtcct-3′ | 19 | 607 | 1485 |

TABLE AOD
Probe Name Ag252

| | | | | SEQ |
|---|---|---|---|---|
| | | | Start | ID |
| Primers | Sequences | Length | Position | No |
| Forward | 5′-gagctgccgcaactcttcc-3′ | 19 | 1453 | 1486 |
| Probe | TET-5′-cgcaactctgcctcttcctcatcgg-3′- | 25 | 1490 | 1487 |
| | TAMRA | | | |
| Reverse | 5′-gacaaacttctctgtgagcgtgtg-3′ | 24 | 1522 | 1488 |

TABLE AOE
Panel 1.3D

| Tissue Name | A |
|---|---|
| Liver adenocarcinoma | 13.9 |
| Pancreas | 4.7 |
| Pancreatic ca. CAPAN 2 | 4.1 |
| Adrenal gland | 6.7 |
| Thyroid | 5.1 |
| Salivary gland | 13.6 |
| Pituitary gland | 5.5 |
| Brain (fetal) | 28.5 |
| Brain (whole) | 43.8 |
| Brain (amygdala) | 32.3 |
| Brain (cerebellum) | 42.9 |
| Brain (hippocampus) | 44.1 |
| Brain (substantia nigra) | 4.1 |
| Brain (thalamus) | 14.8 |
| Cerebral Cortex | 25.5 |
| Spinal cord | 2.6 |
| glio/astro U87-MG | 2.0 |
| glio/astro U-118-MG | 10.2 |
| astrocytoma SW1783 | 10.6 |
| neuro*; met SK-N-AS | 1.7 |
| astrocytoma SF-539 | 4.9 |
| astrocytoma SNB-75 | 10.1 |
| glioma SNB-19 | 6.6 |
| glioma U251 | 8.4 |
| glioma SF-295 | 1.4 |
| Heart (fetal) | 11.8 |
| Heart | 7.6 |
| Skeletal muscle (fetal) | 7.4 |
| Skeletal muscle | 0.0 |
| Bone marrow | 1.8 |
| Thymus | 12.1 |
| Spleen | 0.0 |
| Lymph node | 15.7 |
| Colorectal | 0.0 |
| Stomach | 4.9 |
| Small intestine | 13.7 |
| Colon ca. SW480 | 12.0 |
| Colon ca.* SW620(SW480 met) | 2.9 |
| Colon ca. HT29 | 2.3 |
| Colon ca. HCT-116 | 2.9 |
| Colon ca. CaCo-2 | 2.9 |
| Colon ca. tissue(ODO3866) | 2.9 |
| Colon ca. HCC-2998 | 2.8 |
| Gastric ca.* (liver met) NCI-N87 | 12.9 |
| Bladder | 4.1 |
| Trachea | 34.2 |
| Kidney | 4.7 |
| Kidney (fetal) | 22.8 |
| Renal ca. 786-0 | 0.0 |
| Renal ca. A498 | 19.5 |
| Renal ca. RXF 393 | 53.2 |
| Renal ca. ACHN | 2.4 |
| Renal ca. UO-31 | 1.4 |
| Renal ca. TK-10 | 1.4 |
| Liver | 3.2 |
| Liver (fetal) | 1.4 |
| Liver ca. (hepatoblast) HepG2 | 100.0 |
| Lung | 6.3 |
| Lung (fetal) | 10.2 |
| Lung ca. (small cell) LX-1 | 2.8 |
| Lung ca. (small cell) NCI-H69 | 64.2 |
| Lung ca. (s. cell var.) SHP-77 | 5.9 |
| Lung ca. (large cell)NCI-H460 | 6.4 |
| Lung ca. (non-sm. cell) A549 | 3.3 |
| Lung ca. (non-s. cell) NCI-H23 | 21.6 |
| Lung ca. (non-s. cell) HOP-62 | 11.9 |
| Lung ca. (non-s. cl) NCI-H522 | 40.9 |
| Lung ca. (squam.) SW 900 | 1.7 |
| Lung ca. (squam.) NCI-H596 | 23.3 |
| Mammary gland | 6.9 |
| Breast ca.* (pl. ef) MCF-7 | 6.8 |
| Breast ca.* (pl. ef) MDA-MB-231 | 47.0 |
| Breast ca.* (pl. ef) T47D | 0.0 |
| Breast ca. BT-549 | 22.4 |
| Breast ca. MDA-N | 3.1 |
| Ovary | 0.6 |
| Ovarian ca. OVCAR-3 | 7.1 |
| Ovarian ca. OVCAR-4 | 9.5 |
| Ovarian ca. OVCAR-5 | 2.2 |
| Ovarian ca. OVCAR-8 | 20.6 |
| Ovarian ca. IGROV-1 | 0.0 |
| Ovarian ca.* (ascites) SK-OV-3 | 9.7 |
| Uterus | 6.1 |
| Placenta | 4.9 |
| Prostate | 21.2 |
| Prostate ca.* (bone met)PC-3 | 15.7 |
| Testis | 5.0 |
| Melanoma Hs688(A).T | 1.6 |
| Melanoma* (met) Hs688(B).T | 4.9 |
| Melanoma UACC-62 | 2.7 |
| Melanoma M14 | 3.2 |
| Melanoma LOX IMVI | 3.0 |
| Melanoma* (met) SK-MEL-5 | 1.3 |
| Adipose | 0.0 |

Column A - Rel. Exp. (%) Ag252, Run 165628866

TABLE AOF
Panel 2D

| Tissue Name | A |
|---|---|
| Normal Colon | 11.3 |
| CC Well to Mod Diff (ODO3866) | 11.0 |
| CC Margin (ODO3866) | 1.6 |
| CC Gr.2 rectosigmoid (ODO3868) | 9.7 |
| CC Margin (ODO3868) | 2.5 |
| CC Mod Diff (ODO3920) | 20.9 |
| CC Margin (ODO3920) | 3.0 |
| CC Gr.2 ascend colon (ODO3921) | 3.2 |
| CC Margin (ODO3921) | 1.8 |
| CC from Partial Hepatectomy (ODO4309) Mets | 18.7 |
| Liver Margin (ODO4309) | 2.3 |
| Colon mets to lung (OD04451-01) | 14.0 |
| Lung Margin (OD04451-02) | 17.1 |
| Normal Prostate 6546-1 | 20.6 |
| Prostate Cancer (OD04410) | 30.1 |
| Prostate Margin (OD04410) | 18.4 |
| Prostate Cancer (OD04720-01) | 36.9 |
| Prostate Margin (OD04720-02) | 24.0 |
| Normal Lung 061010 | 15.3 |
| Lung Met to Muscle (ODO4286) | 1.5 |
| Muscle Margin (ODO4286) | 11.1 |
| Lung Malignant Cancer (OD03126) | 23.5 |
| Lung Margin (OD03126) | 19.5 |
| Lung Cancer (OD04404) | 10.5 |
| Lung Margin (OD04404) | 53.2 |
| Lung Cancer (OD04565) | 12.9 |
| Lung Margin (OD04565) | 23.8 |
| Lung Cancer (OD04237-01) | 4.9 |
| Lung Margin (OD04237-02) | 32.5 |
| Ocular Mel Met to Liver (ODO4310) | 2.0 |
| Liver Margin (ODO4310) | 5.4 |
| Melanoma Mets to Lung (OD04321) | 0.7 |
| Lung Margin (OD04321) | 24.5 |
| Normal Kidney | 8.3 |
| Kidney Ca, Nuclear grade 2 (OD04338) | 22.5 |
| Kidney Margin (OD04338) | 4.1 |
| Kidney Ca Nuclear grade 1/2 (OD04339) | 10.9 |
| Kidney Margin (OD04339) | 6.5 |
| Kidney Ca, Clear cell type (OD04340) | 26.8 |
| Kidney Margin (OD04340) | 10.4 |
| Kidney Ca, Nuclear grade 3 (OD04348) | 3.7 |
| Kidney Margin (OD04348) | 6.0 |
| Kidney Cancer (OD04622-01) | 100.0 |
| Kidney Margin (OD04622-03) | 6.5 |
| Kidney Cancer (OD04450-01) | 4.5 |
| Kidney Margin (OD04450-03) | 7.4 |
| Kidney Cancer 8120607 | 3.5 |
| Kidney Margin 8120608 | 8.7 |
| Kidney Cancer 8120613 | 1.2 |
| Kidney Margin 8120614 | 6.1 |
| Kidney Cancer 9010320 | 12.5 |
| Kidney Margin 9010321 | 9.9 |
| Normal Uterus | 18.9 |
| Uterus Cancer 064011 | 15.6 |
| Normal Thyroid | 3.3 |
| Thyroid Cancer 064010 | 6.9 |
| Thyroid Cancer A302152 | 10.9 |
| Thyroid Margin A302153 | 5.7 |
| Normal Breast | 42.3 |
| Breast Cancer (OD04566) | 26.6 |
| Breast Cancer (OD04590-01) | 21.8 |
| Breast Cancer Mets (OD04590-03) | 36.3 |
| Breast Cancer Metastasis (OD04655-05) | 14.9 |
| Breast Cancer 064006 | 21.5 |
| Breast Cancer 1024 | 42.0 |
| Breast Cancer 9100266 | 11.0 |
| Breast Margin 9100265 | 10.8 |
| Breast Cancer A209073 | 14.9 |
| Breast Margin A209073 | 13.0 |
| Normal Liver | 6.4 |
| Liver Cancer 064003 | 0.0 |
| Liver Cancer 1025 | 1.1 |
| Liver Cancer 1026 | 19.6 |
| Liver Cancer 6004-T | 10.0 |
| Liver Tissue 6004-N | 6.0 |
| Liver Cancer 6005-T | 15.3 |
| Liver Tissue 6005-N | 3.3 |
| Normal Bladder | 19.2 |
| Bladder Cancer 1023 | 5.6 |
| Bladder Cancer A302173 | 3.4 |
| Bladder Cancer (OD04718-01) | 7.6 |
| Bladder Normal Adjacent (OD04718-03) | 9.3 |
| Normal Ovary | 2.4 |
| Ovarian Cancer 064008 | 11.8 |
| Ovarian Cancer (OD04768-07) | 2.9 |
| Ovary Margin (OD04768-08) | 31.4 |
| Normal Stomach | 5.6 |
| Gastric Cancer 9060358 | 0.0 |
| Stomach Margin 9060359 | 1.9 |
| Gastric Cancer 9060395 | 5.9 |
| Stomach Margin 9060394 | 2.4 |
| Gastric Cancer 9060397 | 18.6 |
| Stomach Margin 9060396 | 2.8 |
| Gastric Cancer 064005 | 1.5 |

Column A - Rel. Exp. (%) Ag252, Run 144791435

TABLE AOG
Panel 4D

| Tissue Name | A | B | C |
|---|---|---|---|
| Secondary Th1 act | 0.7 | 0.0 | 0.0 |
| Secondary Th2 act | 0.8 | 0.0 | 0.0 |
| Secondary Tr1 act | 0.5 | 0.0 | 4.0 |
| Secondary Th1 rest | 1.7 | 0.0 | 5.6 |
| Secondary Th2 rest | 14.0 | 10.0 | 15.2 |
| Secondary Tr1 rest | 6.2 | 0.0 | 5.7 |
| Primary Th1 act | 1.9 | 2.1 | 1.1 |
| Primary Th2 act | 2.1 | 2.3 | 9.9 |
| Primary Tr1 act | 0.2 | 0.6 | 4.5 |
| Primary Th1 rest | 16.3 | 10.0 | 15.7 |
| Primary Th2 rest | 7.4 | 9.1 | 21.9 |
| Primary Tr1 rest | 11.6 | 11.0 | 13.9 |
| CD45RA CD4 lymphocyte act | 13.7 | 9.4 | 16.6 |
| CD45RO CD4 lymphocyte act | 1.1 | 0.6 | 3.1 |
| CD8 lymphocyte act | 1.5 | 0.9 | 1.1 |
| Secondary CD8 lymphocyte rest | 4.0 | 3.3 | 5.0 |
| Secondary CD8 lymphocyte act | 0.0 | 0.0 | 0.0 |
| CD4 lymphocyte none | 19.8 | 18.6 | 30.8 |
| 2ry Th1/Th2/Tr1_anti-CD95 CH11 | 6.2 | 4.1 | 19.9 |
| LAK cells rest | 8.4 | 8.7 | 17.4 |
| LAK cells IL-2 | 1.6 | 6.2 | 5.8 |
| LAK cells IL-2 + IL-12 | 4.3 | 4.0 | 8.4 |
| LAK cells IL-2 + IFN gamma | 1.0 | 8.1 | 4.1 |
| LAK cells IL-2 + IL-18 | 1.3 | 8.3 | 1.3 |
| LAK cells PMA/ionomycin | 2.6 | 1.2 | 2.5 |
| NK Cells IL-2 rest | 1.2 | 4.8 | 6.8 |
| Two Way MLR 3 day | 8.1 | 13.5 | 3.7 |
| Two Way MLR 5 day | 1.3 | 5.0 | 1.4 |
| Two Way MLR 7 day | 1.0 | 2.0 | 2.6 |
| PBMC rest | 3.0 | 3.7 | 7.8 |
| PBMC PWM | 5.2 | 2.3 | 3.6 |
| PBMC PHA-L | 1.0 | 1.9 | 2.6 |
| Ramos (B cell) none | 0.0 | 0.0 | 0.0 |
| Ramos (B cell) ionomycin | 0.0 | 0.0 | 0.0 |
| B lymphocytes PWM | 1.1 | 1.4 | 1.4 |
| B lymphocytes CD40L and IL-4 | 2.1 | 3.7 | 8.1 |
| EOL-1 dbcAMP | 4.7 | 3.4 | 4.3 |
| EOL-1 dbcAMP PMA/ionomycin | 3.6 | 1.3 | 10.7 |
| Dendritic cells none | 1.7 | 1.9 | 3.3 |
| Dendritic cells LPS | 0.0 | 0.0 | 1.2 |
| Dendritic cells anti-CD40 | 1.6 | 0.6 | 1.8 |
| Monocytes rest | 5.0 | 4.8 | 6.3 |
| Monocytes LPS | 0.4 | 0.6 | 1.4 |
| Macrophages rest | 12.3 | 9.0 | 13.8 |
| Macrophages LPS | 0.5 | 0.6 | 0.0 |
| HUVEC none | 4.2 | 22.5 | 15.6 |
| HUVEC starved | 17.3 | 22.7 | 17.1 |
| HUVEC IL-1beta | 2.9 | 2.0 | 3.5 |
| HUVEC IFN gamma | 27.2 | 18.4 | 25.9 |
| HUVEC TNF alpha + IFN gamma | 8.5 | 9.9 | 2.0 |
| HUVEC TNF alpha + IL4 | 7.9 | 5.9 | 13.7 |
| HUVEC IL-11 | 15.6 | 15.9 | 17.8 |
| Lung Microvascular EC none | 27.7 | 25.7 | 23.8 |
| Lung Microvascular EC TNFalpha + | 24.0 | 22.5 | 19.1 |
| IL-1beta | | | |
| Microvascular Dermal EC none | 14.2 | 15.9 | 17.4 |
| Microsvasular Dermal EC TNFalpha + | 8.2 | 9.4 | 25.3 |
| IL-1beta | | | |
| Bronchial epithelium TNFalpha + | 4.0 | 2.2 | 1.3 |
| IL1beta | | | |
| Small airway epithelium none | 1.9 | 1.6 | 1.5 |
| Small airway epithelium TNFalpha + | 0.5 | 6.4 | 2.6 |
| IL-1beta | | | |
| Coronery artery SMC rest | 23.2 | 29.7 | 33.9 |
| Coronery artery SMC TNFalpha + | 33.4 | 19.5 | 13.5 |
| IL-1beta | | | |
| Astrocytes rest | 0.0 | 26.6 | 17.2 |
| Astrocytes TNFalpha + IL-1beta | 100.0 | 100.0 | 100.0 |
| KU-812 (Basophil) rest | 0.6 | 0.0 | 0.0 |
| KU-812 (Basophil) PMA/ionomycin | 0.0 | 0.5 | 0.0 |
| CCD1106 (Keratinocytes) none | 1.9 | 0.6 | 3.5 |
| CCD1106 (Keratinocytes) TNFalpha + | 0.5 | 2.1 | 3.8 |
| IL-1beta | | | |
| Liver cirrhosis | 9.1 | 12.2 | 13.0 |
| Lupus kidney | 3.0 | 2.6 | 3.8 |
| NCI-H292 none | 30.1 | 41.8 | 25.7 |
| NCI-H292 IL-4 | 16.8 | 35.1 | 16.5 |
| NCI-H292 IL-9 | 21.9 | 28.5 | 32.8 |
| NCI-H292 IL-13 | 37.6 | 28.1 | 33.7 |
| NCI-H292 IFN gamma | 20.3 | 23.5 | 22.5 |
| HPAEC none | 36.1 | 23.5 | 31.6 |
| HPAEC TNF alpha + IL-1 beta | 22.7 | 11.1 | 24.5 |
| Lung fibroblast none | 10.7 | 15.0 | 14.4 |
| Lung fibroblast TNF alpha + IL-1 beta | 1.9 | 2.6 | 1.2 |
| Lung fibroblast IL-4 | 11.0 | 9.7 | 7.7 |
| Lung fibroblast IL-9 | 11.3 | 9.3 | 13.2 |
| Lung fibroblast IL-13 | 7.3 | 4.5 | 17.4 |
| Lung fibroblast IFN gamma | 7.1 | 10.6 | 9.4 |
| Dermal fibroblast CCD1070 rest | 33.2 | 51.4 | 45.4 |
| Dermal fibroblast CCD1070 TNF | 24.0 | 31.9 | 21.3 |
| alpha | | | |
| Dermal fibroblast CCD1070 IL-1 | 34.6 | 34.4 | 46.3 |
| beta | | | |
| Dermal fibroblast IFN gamma | 30.6 | 27.0 | 32.3 |
| Dermal fibroblast IL-4 | 34.4 | 29.7 | 33.9 |
| IBD Colitis 2 | 1.7 | 1.4 | 3.0 |
| IBD Crohn's | 0.7 | 0.0 | 1.3 |
| Colon | 12.9 | 9.2 | 25.0 |
| Lung | 25.7 | 55.5 | 42.9 |
| Thymus | 12.2 | 18.0 | 18.3 |
| Kidney | 26.8 | 39.2 | 25.3 |

Column A - Rel. Exp. (%) Ag1513, Run 163478079
Column B - Rel. Exp. (%) Ag1937, Run 161702009
Column C - Rel. Exp. (%) Ag422, Run 138056654

Panel 1.3D Summary: Ag252 Highest levels of expression of this gene were seen in a liver cell line HepG2(CT=30.27). Based on expression in this panel, this gene is involved in brain, colon, renal, lung, ovarian and prostate cancer as well as melanomas. Thus, expression of this is useful as a diagnostic marker for the presence of these cancers. Therapeutic inhibition using antibodies, protein therapeutics or small molecule drugs are of use in the treatment of these cancers.

This gene product also showed low but significant levels of expression in pancreas, adrenal, thyroid, pituitary, adult and fetal heart, and adult and fetal liver. This gene or its protein product is important for the pathogenesis, diagnosis, and/or treatment of metabolic and endocrine diseases, including obesity and Types 1 and 2 diabetes. Furthermore, this gene was more highly expressed in fetal (CT=34) skeletal muscle when compared to expression in the adult (CT=40) and is useful for the differentiation of the fetal and adult sources of this tissue.

In addition, this gene was expressed at moderate levels in the CNS. This gene encodes a mouse epidermal growth factor homolog, and increases axonal or dendritic outgrowth and synaptogenesis. This gene is of use in the treatment of clinical conditions associated with neuron loss such as head or spinal cord trauma, stroke, or any neurodegenerative disease.

Panel 2D Summary: Ag252 This gene was expressed at low levels in all the samples on this panel, with highest expression in a kidney cancer sample (CT=31.1). Gastric, liver and colon cancers expressed this gene at a higher level than the normal adjacent tissue from these organs. There was increased expression in normal lung and ovarian tissue when compared to the adjacent tumor samples. These data show that the expression of this gene is associated with gastric, liver and colon cancer and thus, therapeutic modulation of this gene product is of use in the treatment of these cancers. Conversely, absence of expression is associated with ovarian and lung cancer and is of use as a diagnostic marker for the presence of these cancers. Therapeutic modulation of this gene is of use in the treatment of these cancers.

Panel 4D Summary: This transcript was expressed at low levels in T cells, fibroblasts, endothelium, smooth muscle cells and T cells regardless of treatment. The transcript was also expressed in normal colon, lung and thymus. However, TNFalpha and IL-1beta induce the expression of the transcript in astrocytes. The trancritpt encodes a Notch like protein which functions in astrocyte differentiation and activation. (Colombatti Glia 2001 September; 35(3):224-33). Therapeutic regulation of this transcript or the design of therapeutics with the encoded protein is important in the treatment of multiple scelrosis or other inflammtory diseases of the CNS.

AP. CG5659401: Claudin-19 Like

Expression of gene CG56594-01 was assessed using the primer-probe set Ag2957, described in Table APA. Results of the RTQ-PCR runs are shown in Tables APB, APC and APD.

TABLE APA
Probe Name Ag2957

| | | | | SEQ |
|---|---|---|---|---|
| | | | Start | ID |
| Primers | Sequences | Length | Position | No |
| Forward | 5′-caggcctctgcactttgac-3′ | 19 | 438 | 1489 |
| Probe | TET-5′-ctgtctcgtggtatgccaccctggt-3′- | 25 | 459 | 1490 |
| | TAMRA | | | |
| Reverse | 5′-ccaaattctgggttgaagaact-3′ | 22 | 492 | 1491 |

TABLE APB
General_screening_panel_v1.4

| Tissue Name | A |
|---|---|
| Adipose | 1.8 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 0.0 |
| Prostate ca.* (bone met) PC-3 | 0.0 |
| Prostate Pool | 0.0 |
| Placenta | 41.5 |
| Uterus Pool | 0.0 |
| Ovarian ca. OVCAR-3 | 0.0 |
| Ovarian ca. SK-OV-3 | 2.7 |
| Ovarian ca. OVCAR-4 | 0.0 |
| Ovarian ca. OVCAR-5 | 0.0 |
| Ovarian ca. IGROV-1 | 0.0 |
| Ovarian ca. OVCAR-8 | 0.0 |
| Ovary | 0.0 |
| Breast ca. MCF-7 | 0.0 |
| Breast ca. MDA-MB-231 | 0.0 |
| Breast ca. BT 549 | 1.7 |
| Breast ca. T47D | 0.0 |
| Breast ca. MDA-N | 0.0 |
| Breast Pool | 0.0 |
| Trachea | 2.8 |
| Lung | 2.9 |
| Fetal Lung | 24.5 |
| Lung ca. NCI-N417 | 0.0 |
| Lung ca. LX-1 | 0.9 |
| Lung ca. NCI-H146 | 0.0 |
| Lung ca. SHP-77 | 0.0 |
| Lung ca. A549 | 0.0 |
| Lung ca. NCI-H526 | 6.0 |
| Lung ca. NCI-H23 | 0.0 |
| Lung ca. NCI-H460 | 0.0 |
| Lung ca. HOP-62 | 0.0 |
| Lung ca. NCI-H522 | 0.0 |
| Liver | 0.0 |
| Fetal Liver | 31.4 |
| Liver ca. HepG2 | 7.6 |
| Kidney Pool | 1.7 |
| Fetal Kidney | 100.0 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 6.0 |
| Bladder | 0.0 |
| Gastric ca. (liver met.) NCI-N87 | 0.0 |
| Gastric ca. KATO III | 0.0 |
| Colon ca. SW-948 | 0.0 |
| Colon ca. SW480 | 0.0 |
| Colon ca.* (SW480 met) SW620 | 4.6 |
| Colon ca. HT29 | 0.0 |
| Colon ca. HCT-116 | 0.0 |
| Colon ca. CaCo-2 | 24.8 |
| Colon cancer tissue | 1.4 |
| Colon ca. SW1116 | 0.0 |
| Colon ca. Colo-205 | 0.0 |
| Colon ca. SW-48 | 0.0 |
| Colon Pool | 0.0 |
| Small Intestine Pool | 0.0 |
| Stomach Pool | 0.0 |
| Bone Marrow Pool | 0.0 |
| Fetal Heart | 0.0 |
| Heart Pool | 0.0 |
| Lymph Node Pool | 0.0 |
| Fetal Skeletal Muscle | 8.0 |
| Skeletal Muscle Pool | 0.0 |
| Spleen Pool | 0.0 |
| Thymus Pool | 3.0 |
| CNS cancer (glio/astro) U87-MG | 0.0 |
| CNS cancer (glio/astro) U-118-MG | 0.0 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) Pool | 0.0 |
| Brain (cerebellum) | 0.0 |
| Brain (fetal) | 1.7 |
| Brain (Hippocampus) Pool | 4.7 |
| Cerebral Cortex Pool | 0.0 |
| Brain (Substantia nigra) Pool | 0.0 |
| Brain (Thalamus) Pool | 0.0 |
| Brain (whole) | 0.0 |
| Spinal Cord Pool | 22.4 |
| Adrenal Gland | 0.0 |
| Pituitary gland Pool | 0.0 |
| Salivary Gland | 0.0 |
| Thyroid (female) | 0.0 |
| Pancreatic ca. CAPAN2 | 2.0 |
| Pancreas Pool | 0.0 |

Column A - Rel. Exp. (%) Ag2957, Run 216861284

TABLE APC
Panel 2D

| Tissue Name | A |
|---|---|
| Normal Colon | 0.0 |
| CC Well to Mod Diff (ODO3866) | 1.0 |
| CC Margin (ODO3866) | 0.0 |
| CC Gr.2 rectosigmoid (ODO3868) | 13.6 |
| CC Margin (ODO3868) | 1.9 |
| CC Mod Diff (ODO3920) | 0.0 |
| CC Margin (ODO3920) | 0.0 |
| CC Gr.2 ascend colon (ODO3921) | 0.0 |
| CC Margin (ODO3921) | 1.2 |
| CC from Partial Hepatectomy (ODO4309) | 0.0 |
| Mets | |
| Liver Margin (ODO4309) | 0.0 |
| Colon mets to lung (OD04451-01) | 0.0 |
| Lung Margin (OD04451-02) | 0.0 |
| Normal Prostate 6546-1 | 1.3 |
| Prostate Cancer (OD04410) | 0.0 |
| Prostate Margin (OD04410) | 2.6 |
| Prostate Cancer (OD04720-01) | 0.0 |
| Prostate Margin (OD04720-02) | 0.0 |
| Normal Lung 061010 | 0.0 |
| Lung Met to Muscle (ODO4286) | 0.0 |
| Muscle Margin (ODO4286) | 0.0 |
| Lung Malignant Cancer (OD03126) | 0.0 |
| Lung Margin (OD03126) | 0.0 |
| Lung Cancer (OD04404) | 0.0 |
| Lung Margin (OD04404) | 0.0 |
| Lung Cancer (OD04565) | 0.0 |
| Lung Margin (OD04565) | 0.0 |
| Lung Cancer (OD04237-01) | 0.0 |
| Lung Margin (OD04237-02) | 0.0 |
| Ocular Mel Met to Liver (ODO4310) | 0.0 |
| Liver Margin (ODO4310) | 1.4 |
| Melanoma Mets to Lung (OD04321) | 0.0 |
| Lung Margin (OD04321) | 0.0 |
| Normal Kidney | 85.3 |
| Kidney Ca, Nuclear grade 2 (OD04338) | 6.2 |
| Kidney Margin (OD04338) | 32.3 |
| Kidney Ca Nuclear grade 1/2 (OD04339) | 13.5 |
| Kidney Margin (OD04339) | 92.0 |
| Kidney Ca, Clear cell type (OD04340) | 9.7 |
| Kidney Margin (OD04340) | 87.7 |
| Kidney Ca, Nuclear grade 3 (OD04348) | 0.0 |
| Kidney Margin (OD04348) | 100.0 |
| Kidney Cancer (OD04622-01) | 0.0 |
| Kidney Margin (OD04622-03) | 50.3 |
| Kidney Cancer (OD04450-01) | 0.0 |
| Kidney Margin (OD04450-03) | 58.6 |
| Kidney Cancer 8120607 | 0.0 |
| Kidney Margin 8120608 | 68.8 |
| Kidney Cancer 8120613 | 0.0 |
| Kidney Margin 8120614 | 24.3 |
| Kidney Cancer 9010320 | 3.4 |
| Kidney Margin 9010321 | 85.9 |
| Normal Uterus | 0.0 |
| Uterus Cancer 064011 | 1.6 |
| Normal Thyroid | 0.0 |
| Thyroid Cancer 064010 | 0.0 |
| Thyroid Cancer A302152 | 0.0 |
| Thyroid Margin A302153 | 0.0 |
| Normal Breast | 3.3 |
| Breast Cancer (OD04566) | 0.0 |
| Breast Cancer (OD04590-01) | 3.0 |
| Breast Cancer Mets (OD04590-03) | 0.0 |
| Breast Cancer Metastasis (OD04655-05) | 0.0 |
| Breast Cancer 064006 | 0.0 |
| Breast Cancer 1024 | 9.5 |
| Breast Cancer 9100266 | 3.3 |
| Breast Margin 9100265 | 5.4 |
| Breast Cancer A209073 | 4.1 |
| Breast Margin A209073 | 4.9 |
| Normal Liver | 0.0 |
| Liver Cancer 064003 | 0.0 |
| Liver Cancer 1025 | 0.0 |
| Liver Cancer 1026 | 0.0 |
| Liver Cancer 6004-T | 0.0 |
| Liver Tissue 6004-N | 12.4 |
| Liver Cancer 6005-T | 5.7 |
| Liver Tissue 6005-N | 0.0 |
| Normal Bladder | 0.0 |
| Bladder Cancer 1023 | 0.0 |
| Bladder Cancer A302173 | 0.0 |
| Bladder Cancer (OD04718-01) | 0.0 |
| Bladder Normal Adjacent (OD04718-03) | 0.0 |
| Normal Ovary | 0.0 |
| Ovarian Cancer 064008 | 0.0 |
| Ovarian Cancer (OD04768-07) | 28.7 |
| Ovary Margin (OD04768-08) | 0.0 |
| Normal Stomach | 0.0 |
| Gastric Cancer 9060358 | 0.0 |
| Stomach Margin 9060359 | 0.0 |
| Gastric Cancer 9060395 | 0.0 |
| Stomach Margin 9060394 | 0.0 |
| Gastric Cancer 9060397 | 0.0 |
| Stomach Margin 9060396 | 0.0 |
| Gastric Cancer 064005 | 0.0 |

Column A - Rel. Exp. (%) Ag2957, Run 170858345

TABLE APD
Panel 4D

| Tissue Name | A |
|---|---|
| Secondary Th1 act | 0.0 |
| Secondary Th2 act | 0.8 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL-1beta | 0.0 |
| Liver cirrhosis | 1.7 |
| Lupus kidney | 5.8 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| IBD Colitis 2 | 0.0 |
| IBD Crohn's | 0.0 |
| Colon | 2.3 |
| Lung | 0.0 |
| Thymus | 100.0 |
| Kidney | 7.6 |

Column A - Rel. Exp. (%) Ag2957, Run 164306319

General_screening_panel_v1.4 Summary: Ag2957 Expression of this gene was restricted to placenta, fetal kidney and liver. Thus, expression of this gene is useful as a marker to differentiate between these samples and other samples on this panel. This gene shows no or very low expression in the cancer cell lines used in this panel and absence of expression of this gene or its protein product is useful as a diagnostic marker for cancer. This gene encodes a putative claudin. Claudins are components of tight junction strands. This specific pattern of expression indicates that this gene product is involved in the formation of TJ strands in these tissues.

Panel 2D Summary: Ag2957 This gene was consistently expressed in the normal kidney samples (CTs=32-33) but not in the adjacent kidney tumors on this panel. Thus, absence of expression of this is useful as a diagnostic marker for kidney cancer. Therapeutic modulation of the function or expression of this gene is useful as a treatment for this cancer.

Panel 4D Summary: Ag2957 The expression of this transcript was restricted to the thymus (CT=32.1) but not in T cells. Expression of this transcript is useful as a marker for this tissue.

AQ. CG5665308: Ficolin 1 Precursor

Expression of gene CG56653-08 was assessed using the primer-probe sets Ag1446, Ag4934 and Ag5886, described in Tables AQA, AQB and AQC. Results of the RTQ-PCR runs are shown in Tables AQD, AQE and AQF.

TABLE AQA
Probe Name Ag1446

| | | | | SEQ |
|---|---|---|---|---|
| | | | Start | ID |
| Primers | Sequences | Length | Position | No |
| Forward | 5′-cgctgtcctgctagtcttgtt-3′ | 21 | 37 | 1492 |
| Probe | TET-5′-atatcaagaacctgcctgcccaggct-3′- | 26 | 63 | 1493 |
| | TAMRA | | | |
| Reverse | 5′-ccttcacctctggacatgtg-3′ | 20 | 94 | 1494 |

TABLE AQB
Probe Name Ag4934

| | | | | SEQ |
|---|---|---|---|---|
| | | | Start | ID |
| Primers | Sequences | Length | Position | No |
| Forward | 5′-cgctgtcctgctagtcttgtt-3′ | 21 | 37 | 1495 |
| Probe | TET-5′-atatcaagaacctgcctgcccaggct-3′- | 26 | 63 | 1496 |
| | TAMRA | | | |
| Reverse | 5′-ccttcacctctggacatgtg-3′ | 20 | 94 | 1497 |

TABLE AQC
3Probe Name Ag5886

| | | | | SEQ |
|---|---|---|---|---|
| | | | Start | ID |
| Primers | Sequences | Length | Position | No |
| Forward | 5′-tggcccgttagagaattacc-3′ | 20 | 734 | 1498 |
| Probe | TET-5′-acaaaggctcccagtaccagcttgta-3′- | 26 | 695 | 1499 |
| | TAMRA | | | |
| Reverse | 5′-caaatcattcaaggtggctg-3′ | 20 | 661 | 1500 |

TABLE AQD
AI_comprehensive panel_v1.0

| Tissue Name | A | B | C |
|---|---|---|---|
| 110967 COPD-F | 3.3 | 1.4 | 0.0 |
| 110980 COPD-F | 4.8 | 1.0 | 1.8 |
| 110968 COPD-M | 3.5 | 0.5 | 1.9 |
| 110977 COPD-M | 14.9 | 6.3 | 5.4 |
| 110989 Emphysema-F | 7.4 | 2.8 | 4.4 |
| 110992 Emphysema-F | 3.6 | 2.2 | 0.2 |
| 110993 Emphysema-F | 6.0 | 1.9 | 0.4 |
| 110994 Emphysema-F | 3.3 | 1.6 | 0.2 |
| 110995 Emphysema-F | 4.3 | 2.1 | 3.3 |
| 110996 Emphysema-F | 1.6 | 1.2 | 0.7 |
| 110997 Asthma-M | 7.6 | 2.6 | 0.6 |
| 111001 Asthma-F | 15.6 | 8.3 | 9.0 |
| 111002 Asthma-F | 20.3 | 6.7 | 6.4 |
| 111003 Atopic Asthma-F | 10.7 | 3.7 | 2.6 |
| 111004 Atopic Asthma-F | 7.2 | 0.0 | 1.7 |
| 111005 Atopic Asthma-F | 3.7 | 1.0 | 1.8 |
| 111006 Atopic Asthma-F | 0.0 | 0.0 | 0.3 |
| 111417 Allergy-M | 4.1 | 1.7 | 1.5 |
| 112347 Allergy-M | 0.4 | 0.0 | 0.2 |
| 112349 Normal Lung-F | 0.5 | 0.0 | 0.1 |
| 112357 Normal Lung-F | 6.7 | 2.8 | 2.2 |
| 112354 Normal Lung-M | 0.5 | 0.4 | 0.6 |
| 112374 Crohns-F | 2.7 | 0.0 | 0.4 |
| 112389 Match Control Crohns-F | 3.5 | 1.7 | 2.4 |
| 112375 Crohns-F | 0.0 | 0.2 | 1.3 |
| 112732 Match Control Crohns-F | 10.2 | 2.8 | 3.2 |
| 112725 Crohns-M | 11.0 | 2.4 | 2.3 |
| 112387 Match Control Crohns-M | 4.9 | 2.3 | 2.7 |
| 112378 Crohns-M | 0.0 | 0.3 | 0.2 |
| 112390 Match Control Crohns-M | 0.8 | 0.3 | 0.6 |
| 112726 Crohns-M | 5.1 | 2.2 | 2.1 |
| 112731 Match Control Crohns-M | 3.1 | 3.3 | 2.1 |
| 112380 Ulcer Col-F | 1.8 | 0.5 | 0.3 |
| 112734 Match Control Ulcer Col-F | 40.6 | 19.1 | 9.7 |
| 112384 Ulcer Col-F | 10.7 | 3.8 | 4.8 |
| 112737 Match Control Ulcer Col-F | 2.0 | 1.5 | 2.0 |
| 112386 Ulcer Col-F | 4.0 | 2.5 | 4.2 |
| 112738 Match Control Ulcer Col-F | 54.3 | 12.0 | 7.7 |
| 112381 Ulcer Col-M | 0.0 | 0.0 | 0.9 |
| 112735 Match Control Ulcer Col-M | 3.4 | 1.8 | 4.3 |
| 112382 Ulcer Col-M | 4.9 | 1.0 | 1.8 |
| 112394 Match Control Ulcer Col-M | 3.7 | 1.4 | 1.9 |
| 112383 Ulcer Col-M | 11.2 | 3.0 | 3.3 |
| 112736 Match Control Ulcer Col-M | 4.6 | 0.5 | 0.7 |
| 112423 Psoriasis-F | 23.3 | 11.7 | 12.9 |
| 112427 Match Control Psoriasis-F | 11.0 | 2.1 | 4.1 |
| 112418 Psoriasis-M | 2.9 | 1.1 | 2.6 |
| 112723 Match Control Psoriasis-M | 0.9 | 0.4 | 0.2 |
| 112419 Psoriasis-M | 5.5 | 2.6 | 3.9 |
| 112424 Match Control Psoriasis-M | 1.8 | 0.9 | 1.5 |
| 112420 Psoriasis-M | 10.2 | 3.3 | 9.2 |
| 112425 Match Control Psoriasis-M | 5.6 | 1.8 | 2.6 |
| 104689 (MF) OA Bone-Backus | 62.0 | 20.3 | 19.9 |
| 104690 (MF) Adj “Normal” | 23.7 | 6.0 | 8.7 |
| Bone-Backus | | | |
| 104691 (MF) OA Synovium-Backus | 10.2 | 5.2 | 3.6 |
| 104692 (BA) OA Cartilage-Backus | 0.8 | 0.0 | 0.3 |
| 104694 (BA) OA Bone-Backus | 49.0 | 16.0 | 11.3 |
| 104695 (BA) Adj “Normal” | 19.9 | 7.9 | 5.1 |
| Bone-Backus | | | |
| 104696 (BA) OA Synovium-Backus | 16.3 | 5.2 | 5.1 |
| 104700 (SS) OA Bone-Backus | 100.0 | 100.0 | 100.0 |
| 104701 (SS) Adj “Normal” | 29.7 | 7.0 | 8.7 |
| Bone-Backus | | | |
| 104702 (SS) OA Synovium-Backus | 27.7 | 9.3 | 11.0 |
| 117093 OA Cartilage Rep7 | 3.3 | 0.0 | 0.8 |
| 112672 OA Bone5 | 23.3 | 5.1 | 10.2 |
| 112673 OA Synovium5 | 7.1 | 2.6 | 2.9 |
| 112674 OA Synovial Fluid cells5 | 9.1 | 3.7 | 4.3 |
| 117100 OA Cartilage Rep14 | 2.4 | 1.3 | 1.5 |
| 112756 OA Bone9 | 3.9 | 0.7 | 0.7 |
| 112757 OA Synovium9 | 2.3 | 2.7 | 0.9 |
| 112758 OA Synovial Fluid Cells9 | 14.3 | 3.6 | 7.3 |
| 117125 RA Cartilage Rep2 | 3.6 | 1.0 | 1.6 |
| 113492 Bone2 RA | 45.4 | 11.2 | 28.7 |
| 113493 Synovium2 RA | 13.9 | 3.5 | 11.8 |
| 113494 Syn Fluid Cells RA | 29.7 | 10.7 | 16.6 |
| 113499 Cartilage4 RA | 14.9 | 6.8 | 10.7 |
| 113500 Bone4 RA | 16.0 | 5.4 | 11.8 |
| 113501 Synovium4 RA | 11.7 | 5.2 | 13.3 |
| 113502 Syn Fluid Cells4 RA | 7.7 | 4.2 | 6.6 |
| 113495 Cartilage3 RA | 26.6 | 12.5 | 16.5 |
| 113496 Bone3 RA | 34.6 | 8.5 | 16.3 |
| 113497 Synovium3 RA | 15.4 | 6.4 | 9.0 |
| 113498 Syn Fluid Cells3 RA | 33.7 | 12.8 | 23.3 |
| 117106 Normal Cartilage Rep20 | 0.5 | 0.6 | 0.9 |
| 113663 Bone3 Normal | 0.0 | 0.3 | 0.2 |
| 113664 Synovium3 Normal | 0.0 | 0.0 | 0.0 |
| 113665 Syn Fluid Cells3 Normal | 0.8 | 0.0 | 0.0 |
| 117107 Normal Cartilage Rep22 | 2.4 | 1.4 | 2.1 |
| 113667 Bone4 Normal | 0.8 | 0.5 | 0.7 |
| 113668 Synovium4 Normal | 0.6 | 0.9 | 0.2 |
| 113669 Syn Fluid Cells4 Normal | 2.4 | 0.4 | 1.0 |

Column A - Rel. Exp. (%) Ag1446, Run 211195015
Column B - Rel. Exp. (%) Ag1446, Run 212650184
Column C - Rel. Exp. (%) Ag5886, Run 256261777

TABLE AQE
General_screening_panel_v1.5

| Tissue Name | A | B |
|---|---|---|
| Adipose | 48.6 | 48.6 |
| Melanoma* Hs688(A).T | 0.0 | 0.0 |
| Melanoma* Hs688(B).T | 0.0 | 0.0 |
| Melanoma* M14 | 0.0 | 0.0 |
| Melanoma* LOXIMVI | 0.0 | 0.0 |
| Melanoma* SK-MEL-5 | 4.8 | 0.0 |
| Squamous cell carcinoma SCC-4 | 0.0 | 0.0 |
| Testis Pool | 1.4 | 1.3 |
| Prostate ca.* (bone met) PC-3 | 0.0 | 0.0 |
| Prostate Pool | 9.3 | 5.6 |
| Placenta | 26.8 | 15.1 |
| Uterus Pool | 12.4 | 4.9 |
| Ovarian ca. OVCAR-3 | 0.0 | 0.0 |
| Ovarian ca. SK-OV-3 | 0.0 | 0.0 |
| Ovarian ca. OVCAR-4 | 0.0 | 0.0 |
| Ovarian ca. OVCAR-5 | 0.0 | 0.0 |
| Ovarian ca. IGROV-1 | 0.0 | 0.0 |
| Ovarian ca. OVCAR-8 | 0.0 | 0.0 |
| Ovary | 4.6 | 6.3 |
| Breast ca. MCF-7 | 0.0 | 0.0 |
| Breast ca. MDA-MB-231 | 0.0 | 0.0 |
| Breast ca. BT 549 | 0.0 | 0.0 |
| Breast ca. T47D | 0.0 | 0.0 |
| Breast ca. MDA-N | 0.0 | 0.0 |
| Breast Pool | 25.3 | 15.9 |
| Trachea | 24.3 | 10.7 |
| Lung | 0.7 | 0.7 |
| Fetal Lung | 100.0 | 100.0 |
| Lung ca. NCI-N417 | 0.0 | 0.0 |
| Lung ca. LX-1 | 0.0 | 0.0 |
| Lung ca. NCI-H146 | 0.0 | 0.0 |
| Lung ca. SHP-77 | 0.0 | 0.0 |
| Lung ca. A549 | 0.0 | 0.0 |
| Lung ca. NCI-H526 | 0.0 | 0.0 |
| Lung ca. NCI-H23 | 0.0 | 0.0 |
| Lung ca. NCI-H460 | 0.0 | 0.0 |
| Lung ca. HOP-62 | 0.0 | 0.0 |
| Lung ca. NCI-H522 | 0.0 | 0.0 |
| Liver | 2.2 | 3.3 |
| Fetal Liver | 28.5 | 12.6 |
| Liver ca. HepG2 | 0.0 | 0.0 |
| Kidney Pool | 18.6 | 7.9 |
| Fetal Kidney | 7.5 | 9.5 |
| Renal ca. 786-0 | 0.0 | 0.0 |
| Renal ca. A498 | 0.0 | 0.0 |
| Renal ca. ACHN | 0.0 | 0.0 |
| Renal ca. UO-31 | 0.0 | 0.0 |
| Renal ca. TK-10 | 0.0 | 0.0 |
| Bladder | 6.4 | 4.0 |
| Gastric ca. (liver met.) NCI-N87 | 0.0 | 0.0 |
| Gastric ca. KATO III | 0.0 | 0.0 |
| Colon ca. SW-948 | 0.0 | 0.0 |
| Colon ca. SW480 | 0.0 | 0.0 |
| Colon ca.* (SW480 met) SW620 | 0.0 | 0.0 |
| Colon ca. HT29 | 0.0 | 0.0 |
| Colon ca. HCT-116 | 0.0 | 0.0 |
| Colon ca. CaCo-2 | 0.0 | 0.0 |
| Colon cancer tissue | 35.6 | 14.9 |
| Colon ca. SW1116 | 0.0 | 0.0 |
| Colon ca. Colo-205 | 0.0 | 0.0 |
| Colon ca. SW-48 | 0.0 | 0.0 |
| Colon Pool | 68.8 | 34.4 |
| Small Intestine Pool | 9.4 | 3.8 |
| Stomach Pool | 7.5 | 4.8 |
| Bone Marrow Pool | 8.5 | 8.4 |
| Fetal Heart | 5.4 | 13.2 |
| Heart Pool | 9.9 | 3.4 |
| Lymph Node Pool | 8.9 | 9.5 |
| Fetal Skeletal Muscle | 7.2 | 12.6 |
| Skeletal Muscle Pool | 17.4 | 5.2 |
| Spleen Pool | 84.7 | 60.7 |
| Thymus Pool | 16.8 | 6.0 |
| CNS cancer (glio/astro) U87-MG | 0.0 | 0.0 |
| CNS cancer (glio/astro) U-118-MG | 0.0 | 0.0 |
| CNS cancer (neuro; met) SK-N-AS | 0.0 | 0.0 |
| CNS cancer (astro) SF-539 | 0.0 | 0.0 |
| CNS cancer (astro) SNB-75 | 0.0 | 0.0 |
| CNS cancer (glio) SNB-19 | 0.0 | 0.0 |
| CNS cancer (glio) SF-295 | 0.0 | 0.1 |
| Brain (Amygdala) Pool | 1.8 | 0.2 |
| Brain (cerebellum) | 1.4 | 1.1 |
| Brain (fetal) | 3.6 | 1.1 |
| Brain (Hippocampus) Pool | 2.0 | 0.8 |
| Cerebral Cortex Pool | 1.9 | 0.5 |
| Brain (Substantia nigra) Pool | 1.7 | 0.9 |
| Brain (Thalamus) Pool | 1.4 | 0.9 |
| Brain (whole) | 3.7 | 2.7 |
| Spinal Cord Pool | 3.1 | 0.6 |
| Adrenal Gland | 6.0 | 4.1 |
| Pituitary gland Pool | 0.7 | 0.7 |
| Salivary Gland | 5.9 | 2.7 |
| Thyroid (female) | 15.0 | 5.5 |
| Pancreatic ca. CAPAN2 | 0.0 | 0.0 |
| Pancreas Pool | 27.9 | 9.7 |

Column A - Rel. Exp. (%) Ag4934, Run 228843453
Column B - Rel. Exp. (%) Ag5886, Run 247756990

TABLE AQF
Panel 4.1D

| Tissue Name | A | B |
|---|---|---|
| Secondary Th1 act | 0.0 | 0.0 |
| Secondary Th2 act | 0.0 | 0.0 |
| Secondary Tr1 act | 0.0 | 0.0 |
| Secondary Th1 rest | 0.0 | 0.0 |
| Secondary Th2 rest | 0.0 | 0.0 |
| Secondary Tr1 rest | 0.0 | 0.0 |
| Primary Th1 act | 0.0 | 0.0 |
| Primary Th2 act | 0.0 | 0.0 |
| Primary Tr1 act | 0.0 | 0.0 |
| Primary Th1 rest | 0.0 | 0.0 |
| Primary Th2 rest | 0.0 | 0.0 |
| Primary Tr1 rest | 0.0 | 0.0 |
| CD45RA CD4 lymphocyte act | 0.0 | 0.0 |
| CD45RO CD4 lymphocyte act | 0.0 | 0.0 |
| CD8 lymphocyte act | 0.0 | 0.0 |
| Secondary CD8 lymphocyte rest | 0.0 | 0.0 |
| Secondary CD8 lymphocyte act | 0.0 | 0.0 |
| CD4 lymphocyte none | 0.5 | 0.3 |
| 2ry Th1/Th2/Tr1_anti-CD95 CH11 | 0.0 | 0.0 |
| LAK cells rest | 6.9 | 21.5 |
| LAK cells IL-2 | 0.0 | 0.0 |
| LAK cells IL-2 + IL-12 | 0.0 | 0.0 |
| LAK cells IL-2 + IFN gamma | 0.0 | 0.0 |
| LAK cells IL-2 + IL-18 | 0.0 | 0.0 |
| LAK cells PMA/ionomycin | 8.2 | 66.0 |
| NK Cells IL-2 rest | 0.0 | 0.0 |
| Two Way MLR 3 day | 1.0 | 1.7 |
| Two Way MLR 5 day | 0.1 | 0.0 |
| Two Way MLR 7 day | 0.0 | 0.0 |
| PBMC rest | 19.2 | 24.0 |
| PBMC PWM | 0.0 | 0.0 |
| PBMC PHA-L | 0.0 | 0.0 |
| Ramos (B cell) none | 0.0 | 0.0 |
| Ramos (B cell) ionomycin | 0.0 | 0.0 |
| B lymphocytes PWM | 0.0 | 0.0 |
| B lymphocytes CD40L and IL-4 | 0.0 | 0.1 |
| EOL-1 dbcAMP | 3.5 | 14.9 |
| EOL-1 dbcAMP PMA/ionomycin | 0.5 | 0.2 |
| Dendritic cells none | 1.0 | 4.5 |
| Dendritic cells LPS | 0.1 | 0.0 |
| Dendritic cells anti-CD40 | 0.6 | 0.1 |
| Monocytes rest | 100.0 | 100.0 |
| Monocytes LPS | 4.8 | 15.6 |
| Macrophages rest | 4.1 | 5.0 |
| Macrophages LPS | 1.8 | 2.3 |
| HUVEC none | 0.0 | 0.0 |
| HUVEC starved | 0.0 | 0.0 |
| HUVEC IL-1beta | 0.0 | 0.0 |
| HUVEC IFN gamma | 0.0 | 0.0 |
| HUVEC TNF alpha + IFN gamma | 0.0 | 0.0 |
| HUVEC TNF alpha + IL4 | 0.0 | 0.0 |
| HUVEC IL-11 | 0.0 | 0.0 |
| Lung Microvascular EC none | 0.0 | 0.0 |
| Lung Microvascular EC TNFalpha + IL-1beta | 0.0 | 0.0 |
| Microvascular Dermal EC none | 0.0 | 0.0 |
| Microsvasular Dermal EC TNFalpha + IL-1beta | 0.0 | 0.0 |
| Bronchial epithelium TNFalpha + IL1beta | 0.0 | 0.0 |
| Small airway epithelium none | 0.0 | 0.0 |
| Small airway epithelium TNFalpha + IL-1beta | 0.0 | 0.0 |
| Coronery artery SMC rest | 0.0 | 0.0 |
| Coronery artery SMC TNFalpha + IL-1beta | 0.0 | 0.0 |
| Astrocytes rest | 0.0 | 0.0 |
| Astrocytes TNFalpha + IL-1beta | 0.0 | 0.0 |
| KU-812 (Basophil) rest | 0.0 | 0.0 |
| KU-812 (Basophil) PMA/ionomycin | 0.0 | 0.0 |
| CCD1106 (Keratinocytes) none | 0.0 | 0.0 |
| CCD1106 (Keratinocytes) TNFalpha + IL-1beta | 0.0 | 0.0 |
| Liver cirrhosis | 0.2 | 0.8 |
| NCI-H292 none | 0.0 | 0.0 |
| NCI-H292 IL-4 | 0.0 | 0.0 |
| NCI-H292 IL-9 | 0.0 | 0.0 |
| NCI-H292 IL-13 | 0.0 | 0.0 |
| NCI-H292 IFN gamma | 0.0 | 0.0 |
| HPAEC none | 0.0 | 0.0 |
| HPAEC TNF alpha + IL-1 beta | 0.0 | 0.0 |
| Lung fibroblast none | 0.0 | 0.0 |
| Lung fibroblast TNF alpha + IL-1 beta | 0.0 | 0.0 |
| Lung fibroblast IL-4 | 0.0 | 0.0 |
| Lung fibroblast IL-9 | 0.0 | 0.0 |
| Lung fibroblast IL-13 | 0.0 | 0.0 |
| Lung fibroblast IFN gamma | 0.0 | 0.0 |
| Dermal fibroblast CCD1070 rest | 0.0 | 0.0 |
| Dermal fibroblast CCD1070 TNF alpha | 0.0 | 0.0 |
| Dermal fibroblast CCD1070 IL-1 beta | 0.1 | 0.0 |
| Dermal fibroblast IFN gamma | 0.1 | 0.0 |
| Dermal fibroblast IL-4 | 0.3 | 0.0 |
| Dermal Fibroblasts rest | 0.1 | 0.0 |
| Neutrophils TNFa + LPS | 1.8 | 14.0 |
| Neutrophils rest | 3.8 | 47.3 |
| Colon | 0.0 | 0.0 |
| Lung | 0.3 | 0.2 |
| Thymus | 0.2 | 0.1 |
| Kidney | 0.1 | 0.2 |

Column A - Rel. Exp. (%) Ag4934, Run 223597255
Column B - Rel. Exp. (%) Ag5886, Run 256523048

AI_comprehensive panel_v1.0 Summary: Ag1446/Ag5886 Highest expression of this gene was seen in a bone sample from an OA patient (CTs=27-30.4). Moderate to high expression of this gene wass mainly seen in bone, synovium, synovial fluid and cartilage of OA and RA patients. Expression of this gene was low to undetectable in normal bone. Moderate to low expression was also found in colon, asthma, atopic asthma, emphysema, COPD and psoriasis samples. Ficolins are multimeric lectins that are capable of binding to bacteria and extracellular matrix proteins [Ohashi T. J Biol Chem 1997 May 30;272(22):14220-6]. Ficolin has been reported to function as a monocyte cell surface molecule that is important for binding to bacteria, elastin and for monocyte adhesion [Lu J, Immunobiology 1998 August;199(2):190-9]. This expression proves that ficolin is useful in alleviating inflammation in joints and other sites of inflammation. This gene functions as a protein therapeutic to reduce inflammation in osteoarthritis and rheumatoid arthritis. Ficolin also a role in the inflammation of joints in patients suffering from osteoarthritis (OA) and/or rheumatoid arthritis (RA). Antibodies against proteins encoded by this gene are useful for the prevention of tissue destruction mediated by ficolin activity during osteoarthritis and arthritis.

General_screening_panel_v1.5 Summary: Ag4934/Ag5886 Highest expression of this gene was detected in the fetal lung (CTs=27-31.5). Expression of this gene was also higher in fetal lung and fetal liver (CTs=27-33) than in their adult counterparts (CTs=32-38). Expression of this gene is useful as a marker to distinguish fetal from adult lung and liver. The relative overexpression of this gene in fetal tissue shows that the protein product enhances lung and liver growth or development in the fetus and also acts in a regenerative capacity in the adult. Therapeutic modulation of the protein encoded by this gene is useful in treatment of liver related diseases. Low but significant levels of expression was detected in tissues with metabolic/endocrine functions including adipose, heart, skeletal muscle, thyroid, pancreas and gastrointestinal tracts. Modulation of this gene or gene product is a treatment for metabolic or endocrine disease including obesity and Types 1 and 2 diabetes.

Low expression of this gene was also seen in some regions of central nervous system including whole and fetal brain, cerebellum, hipocampus, substantia nigra, thalamus and spinal cord. Therapeutic modulation of this gene or gene product is useful in the treatment of central nervous system disorders such as Alzheimer's disease, Parkinson's disease, epilepsy, multiple sclerosis, schizophrenia and depression.

Panel 4.1D Summary: Ag4934/Ag5886 This gene was highly and selectively expressed in resting monocytes and to a lesser extent in macrophages and granulocytes (neutrophils and EOL cell line). This gene encodes a putative ficolin 1 precursor. Ficolin functions as a monocyte cell surface molecule that is important for binding to bacteria, elastin and monocyte adhesion. Protein therapeutics, antibodies, or small molecules designed with the protein encoded by this transcript are useful as opsinins to target and eliminate bacteria by complement-mediated destruction. These proteins are also important for the treatment of bacterial septicemia. In addition, ficolins may have the ability to bind to elastins. Elastins are functionally important for lung alveolar development and inactivation of these proteins can lead to emphysema-like disease. Therefore, antibodies, small molecules or protein therapeutics that target this gene or its protein product are useful for the prevention of tissue destruction mediated by ficolin activity during emphysema, asthma and arthritis.

AR. CG56806-01: Heparan Sulfate 6-Sulfotransferase 3 Like Gene

Expression of gene CG56806-01 was assessed using the primer-probe set Ag3024, described in Table ARA. Results of the RTQ-PCR runs are shown in Tables ARB, ARC, ARD, ARE and ARF.

TABLE ARA
Probe Name Ag3024

| | | | | SEQ |
|---|---|---|---|---|
| | | | Start | ID |
| Primers | Sequences | Length | Position | No |
| Forward | 5′-ggtgggctgctataacttgact-3′ | 22 | 1033 | 1501 |
| Probe | TET-5′-tgaaagaaacaccatcctgttgcaga-3′- | 26 | 1069 | 1502 |
| | TAMRA | | | |
| Reverse | 5′-tgttcttcaggttgttctttgc-3′ | 22 | 1097 | 1503 |

TABLE ARB
AI_comprehensive panel_v1.0

| Tissue Name | A |
|---|---|
| 110967 COPD-F | 10.6 |
| 110980 COPD-F | 9.5 |
| 110968 COPD-M | 10.8 |
| 110977 COPD-M | 16.7 |
| 110989 Emphysema-F | 44.8 |
| 110992 Emphysema-F | 3.8 |
| 110993 Emphysema-F | 2.6 |
| 110994 Emphysema-F | 1.0 |
| 110995 Emphysema-F | 9.0 |
| 110996 Emphysema-F | 1.3 |
| 110997 Asthma-M | 1.0 |
| 111001 Asthma-F | 33.4 |
| 111002 Asthma-F | 37.9 |
| 111003 Atopic Asthma-F | 41.5 |
| 111004 Atopic Asthma-F | 21.2 |
| 111005 Atopic Asthma-F | 39.0 |
| 111006 Atopic Asthma-F | 8.5 |
| 111417 Allergy-M | 26.6 |
| 112347 Allergy-M | 8.6 |
| 112349 Normal Lung-F | 9.2 |
| 112357 Normal Lung-F | 0.6 |
| 112354 Normal Lung-M | 1.3 |
| 112374 Crohns-F | 4.0 |
| 112389 Match Control Crohns-F | 1.3 |
| 112375 Crohns-F | 4.5 |
| 112732 Match Control Crohns-F | 0.0 |
| 112725 Crohns-M | 9.9 |
| 112387 Match Control Crohns-M | 15.3 |
| 112378 Crohns-M | 9.7 |
| 112390 Match Control Crohns-M | 23.7 |
| 112726 Crohns-M | 2.9 |
| 112731 Match Control Crohns-M | 1.4 |
| 112380 Ulcer Col-F | 8.7 |
| 112734 Match Control Ulcer Col-F | 0.7 |
| 112384 Ulcer Col-F | 25.3 |
| 112737 Match Control Ulcer Col-F | 3.0 |
| 112386 Ulcer Col-F | 3.3 |
| 112738 Match Control Ulcer Col-F | 0.6 |
| 112381 Ulcer Col-M | 1.8 |
| 112735 Match Control Ulcer Col-M | 32.8 |
| 112382 Ulcer Col-M | 1.5 |
| 112394 Match Control Ulcer Col-M | 1.8 |
| 112383 Ulcer Col-M | 6.3 |
| 112736 Match Control Ulcer Col-M | 1.1 |
| 112423 Psoriasis-F | 12.7 |
| 112427 Match Control Psoriasis-F | 37.1 |
| 112418 Psoriasis-M | 6.1 |
| 112723 Match Control Psoriasis-M | 0.0 |
| 112419 Psoriasis-M | 16.6 |
| 112424 Match Control Psoriasis-M | 17.8 |
| 112420 Psoriasis-M | 100.0 |
| 112425 Match Control Psoriasis-M | 26.2 |
| 104689 (MF) OA Bone-Backus | 0.0 |
| 104690 (MF) Adj “Normal” Bone-Backus | 0.4 |
| 104691 (MF) OA Synovium-Backus | 3.0 |
| 104692 (BA) OA Cartilage-Backus | 1.0 |
| 104694 (BA) OA Bone-Backus | 0.0 |
| 104695 (BA) Adj “Normal” Bone-Backus | 0.6 |
| 104696 (BA) OA Synovium-Backus | 0.5 |
| 104700 (SS) OA Bone-Backus | 0.2 |
| 104701 (SS) Adj “Normal” Bone-Backus | 0.3 |
| 104702 (SS) OA Synovium-Backus | 4.5 |
| 117093 OA Cartilage Rep7 | 42.0 |
| 112672 OA Bone5 | 18.4 |
| 112673 OA Synovium5 | 8.0 |
| 112674 OA Synovial Fluid cells5 | 2.4 |
| 117100 OA Cartilage Rep14 | 5.5 |
| 112756 OA Bone9 | 3.5 |
| 112757 OA Synovium9 | 2.1 |
| 112758 OA Synovial Fluid Cells9 | 11.9 |
| 117125 RA Cartilage Rep2 | 2.5 |
| 113492 Bone2 RA | 1.7 |
| 113493 Synovium2 RA | 0.0 |
| 113494 Syn Fluid Cells RA | 1.6 |
| 113499 Cartilage4 RA | 0.4 |
| 113500 Bone4 RA | 1.3 |
| 113501 Synovium4 RA | 0.0 |
| 113502 Syn Fluid Cells4 RA | 0.2 |
| 113495 Cartilage3 RA | 0.2 |
| 113496 Bone3 RA | 0.6 |
| 113497 Synovium3 RA | 0.9 |
| 113498 Syn Fluid Cells3 RA | 0.6 |
| 117106 Normal Cartilage Rep20 | 14.1 |
| 113663 Bone3 Normal | 8.5 |
| 113664 Synovium3 Normal | 4.0 |
| 113665 Syn Fluid Cells3 Normal | 5.7 |
| 117107 Normal Cartilage Rep22 | 13.8 |
| 113667 Bone4 Normal | 17.2 |
| 113668 Synovium4 Normal | 14.7 |
| 113669 Syn Fluid Cells4 Normal | 7.6 |

Column A - Rel. Exp. (%) Ag3024, Run 248122026

TABLE ARC
Panel 1.3D

| Tissue Name | A |
|---|---|
| Liver adenocarcinoma | 0.4 |
| Pancreas | 0.0 |
| Pancreatic ca. CAPAN 2 | 0.0 |
| Adrenal gland | 2.6 |
| Thyroid | 0.7 |
| Salivary gland | 0.0 |
| Pituitary gland | 2.0 |
| Brain (fetal) | 11.0 |
| Brain (whole) | 54.7 |
| Brain (amygdala) | 27.2 |
| Brain (cerebellum) | 12.9 |
| Brain (hippocampus) | 36.3 |
| Brain (substantia nigra) | 5.7 |
| Brain (thalamus) | 64.2 |
| Cerebral Cortex | 100.0 |
| Spinal cord | 3.8 |
| glio/astro U87-MG | 0.0 |
| glio/astro U-118-MG | 14.7 |
| astrocytoma SW1783 | 1.7 |
| neuro*; met SK-N-AS | 0.0 |
| astrocytoma SF-539 | 0.0 |
| astrocytoma SNB-75 | 6.0 |
| glioma SNB-19 | 0.6 |
| glioma U251 | 0.0 |
| glioma SF-295 | 0.0 |
| Heart (fetal) | 0.0 |
| Heart | 0.6 |
| Skeletal muscle (fetal) | 1.9 |
| Skeletal muscle | 0.4 |
| Bone marrow | 0.0 |
| Thymus | 0.0 |
| Spleen | 0.7 |
| Lymph node | 0.0 |
| Colorectal | 0.8 |
| Stomach | 6.9 |
| Small intestine | 1.4 |
| Colon ca. SW480 | 0.0 |
| Colon ca.* SW620(SW480 met) | 0.0 |
| Colon ca. HT29 | 0.0 |
| Colon ca. HCT-116 | 0.0 |
| Colon ca. CaCo-2 | 0.0 |
| Colon ca. tissue(ODO3866) | 1.0 |
| Colon ca. HCC-2998 | 0.0 |
| Gastric ca.* (liver met) NCI-N87 | 0.4 |
| Bladder | 0.7 |
| Trachea | 2.4 |
| Kidney | 0.6 |
| Kidney (fetal) | 0.2 |
| Renal ca. 786-0 | 0.0 |
| Renal ca. A498 | 2.4 |
| Renal ca. RXF 393 | 0.0 |
| Renal ca. ACHN | 0.0 |
| Renal ca. UO-31 | 0.0 |
| Renal ca. TK-10 | 0.0 |
| Liver | 0.7 |
| Liver (fetal) | 0.0 |
| Liver ca. (hepatoblast) HepG2 | 0.0 |
| Lung | 0.0 |
| Lung (fetal) | 0.0 |
| Lung ca. (small cell) LX-1 | 0.0 |
| Lung ca. (small cell) NCI-H69 | 0.0 |
| Lung ca. (s. cell var.) SHP-77 | 0.0 |
| Lung ca. (large cell)NCI-H460 | 0.0 |
| Lung ca. (non-sm. cell) A549 | 0.2 |
| Lung ca. (non-s. cell) NCI-H23 | 0.0 |
| Lung ca. (non-s. cell) HOP-62 | 0.6 |
| Lung ca. (non-s. cl) NCI-H522 | 0.0 |
| Lung ca. (squam.) SW 900 | 2.1 |
| Lung ca. (squam.) NCI-H596 | 0.0 |
| Mammary gland | 0.4 |
| Breast ca.* (pl. ef) MCF-7 | 11.0 |
| Breast ca.* (pl. ef) MDA-MB-231 | 0.9 |
| Breast ca.* (pl. ef) T47D | 2.3 |
| Breast ca. BT-549 | 1.0 |
| Breast ca. MDA-N | 0.0 |
| Ovary | 0.0 |
| Ovarian ca. OVCAR-3 | 0.0 |
| Ovarian ca. OVCAR-4 | 0.0 |
| Ovarian ca. OVCAR-5 | 0.2 |
| Ovarian ca. OVCAR-8 | 0.0 |
| Ovarian ca. IGROV-1 | 0.0 |
| Ovarian ca.* (ascites) SK-OV-3 | 0.0 |
| Uterus | 2.2 |
| Placenta | 0.0 |
| Prostate | 0.4 |
| Prostate ca.* (bone met)PC-3 | 0.0 |
| Testis | 1.7 |
| Melanoma Hs688(A).T | 0.0 |
| Melanoma* (met) Hs688(B).T | 0.0 |
| Melanoma UACC-62 | 0.2 |
| Melanoma M14 | 0.0 |
| Melanoma LOX IMVI | 0.0 |
| Melanoma* (met) SK-MEL-5 | 0.0 |
| Adipose | 0.0 |

Column A - Rel. Exp. (%) Ag3024, Run 165517896

TABLE ARD
Panel 2D

| Tissue Name | A |
|---|---|
| Normal Colon | 4.7 |
| CC Well to Mod Diff (ODO3866) | 5.0 |
| CC Margin (ODO3866) | 3.1 |
| CC Gr.2 rectosigmoid (ODO3868) | 0.0 |
| CC Margin (ODO3868) | 0.3 |
| CC Mod Diff (ODO3920) | 0.0 |
| CC Margin (ODO3920) | 0.5 |
| CC Gr.2 ascend colon (ODO3921) | 3.1 |
| CC Margin (ODO3921) | 2.1 |
| CC from Partial Hepatectomy (ODO4309) Mets | 1.8 |
| Liver Margin (ODO4309) | 0.0 |
| Colon mets to lung (OD04451-01) | 0.0 |
| Lung Margin (OD04451-02) | 0.0 |
| Normal Prostate 6546-1 | 3.7 |
| Prostate Cancer (OD04410) | 5.2 |
| Prostate Margin (OD04410) | 5.4 |
| Prostate Cancer (OD04720-01) | 11.9 |
| Prostate Margin (OD04720-02) | 6.4 |
| Normal Lung 061010 | 0.0 |
| Lung Met to Muscle (ODO4286) | 0.6 |
| Muscle Margin (ODO4286) | 1.0 |
| Lung Malignant Cancer (OD03126) | 0.0 |
| Lung Margin (OD03126) | 1.6 |
| Lung Cancer (OD04404) | 0.0 |
| Lung Margin (OD04404) | 1.0 |
| Lung Cancer (OD04565) | 0.0 |
| Lung Margin (OD04565) | 0.0 |
| Lung Cancer (OD04237-01) | 8.4 |
| Lung Margin (OD04237-02) | 0.0 |
| Ocular Mel Met to Liver (ODO4310) | 2.5 |
| Liver Margin (ODO4310) | 0.0 |
| Melanoma Mets to Lung (OD04321) | 0.4 |
| Lung Margin (OD04321) | 0.5 |
| Normal Kidney | 23.7 |
| Kidney Ca, Nuclear grade 2 (OD04338) | 1.2 |
| Kidney Margin (OD04338) | 10.6 |
| Kidney Ca Nuclear grade 1/2 (OD04339) | 0.0 |
| Kidney Margin (OD04339) | 7.7 |
| Kidney Ca, Clear cell type (OD04340) | 9.3 |
| Kidney Margin (OD04340) | 17.2 |
| Kidney Ca, Nuclear grade 3 (OD04348) | 2.3 |
| Kidney Margin (OD04348) | 5.1 |
| Kidney Cancer (OD04622-01) | 0.0 |
| Kidney Margin (OD04622-03) | 2.8 |
| Kidney Cancer (OD04450-01) | 0.0 |
| Kidney Margin (OD04450-03) | 17.8 |
| Kidney Cancer 8120607 | 1.7 |
| Kidney Margin 8120608 | 1.3 |
| Kidney Cancer 8120613 | 100.0 |
| Kidney Margin 8120614 | 5.3 |
| Kidney Cancer 9010320 | 0.8 |
| Kidney Margin 9010321 | 6.3 |
| Normal Uterus | 0.7 |
| Uterus Cancer 064011 | 9.4 |
| Normal Thyroid | 5.7 |
| Thyroid Cancer 064010 | 0.3 |
| Thyroid Cancer A302152 | 2.0 |
| Thyroid Margin A302153 | 23.5 |
| Normal Breast | 7.9 |
| Breast Cancer (OD04566) | 7.4 |
| Breast Cancer (OD04590-01) | 0.0 |
| Breast Cancer Mets (OD04590-03) | 3.6 |
| Breast Cancer Metastasis (OD04655-05) | 3.8 |
| Breast Cancer 064006 | 7.1 |
| Breast Cancer 1024 | 6.2 |
| Breast Cancer 9100266 | 27.7 |
| Breast Margin 9100265 | 4.3 |
| Breast Cancer A209073 | 30.8 |
| Breast Margin A209073 | 3.0 |
| Normal Liver | 1.3 |
| Liver Cancer 064003 | 0.4 |
| Liver Cancer 1025 | 0.0 |
| Liver Cancer 1026 | 0.0 |
| Liver Cancer 6004-T | 0.0 |
| Liver Tissue 6004-N | 1.1 |
| Liver Cancer 6005-T | 0.0 |
| Liver Tissue 6005-N | 0.0 |
| Normal Bladder | 2.8 |
| Bladder Cancer 1023 | 0.0 |
| Bladder Cancer A302173 | 2.2 |
| Bladder Cancer (OD04718-01) | 0.7 |
| Bladder Normal Adjacent (OD04718-03) | 0.4 |
| Normal Ovary | 0.0 |
| Ovarian Cancer 064008 | 1.4 |
| Ovarian Cancer (OD04768-07) | 0.0 |
| Ovary Margin (OD04768-08) | 3.3 |
| Normal Stomach | 17.8 |
| Gastric Cancer 9060358 | 1.0 |
| Stomach Margin 9060359 | 13.6 |
| Gastric Cancer 9060395 | 7.5 |
| Stomach Margin 9060394 | 4.1 |
| Gastric Cancer 9060397 | 13.4 |
| Stomach Margin 9060396 | 6.7 |
| Gastric Cancer 064005 | 1.2 |

Column A - Rel. Exp. (%) Ag3024, Run 163577593

TABLE ARE
Panel 3D

| Tissue Name | A |
|---|---|
| Daoy- Medulloblastoma | 1.6 |
| TE671- Medulloblastoma | 0.0 |
| D283 Med- Medulloblastoma | 0.0 |
| PFSK-1- Primitive Neuroectodermal | 0.0 |
| XF-498- CNS | 0.6 |
| SNB-78- Glioma | 0.0 |
| SF-268- Glioblastoma | 0.4 |
| T98G- Glioblastoma | 14.1 |
| SK-N-SH- Neuroblastoma (metastasis) | 0.2 |
| SF-295- Glioblastoma | 0.4 |
| Cerebellum | 15.3 |
| Cerebellum | 5.4 |
| NCI-H292- Mucoepidermoid lung carcinoma | 0.0 |
| DMS-114- Small cell lung cancer | 0.0 |
| DMS-79- Small cell lung cancer | 3.3 |
| NCI-H146- Small cell lung cancer | 0.0 |
| NCI-H526- Small cell lung cancer | 57.4 |
| NCI-N417- Small cell lung cancer | 14.4 |
| NCI-H82- Small cell lung cancer | 0.0 |
| NCI-H157- Squamous cell lung cancer | 0.0 |
| (metastasis) | |
| NCI-H1155- Large cell lung cancer | 2.9 |
| NCI-H1299- Large cell lung cancer | 0.3 |
| NCI-H727- Lung carcinoid | 15.9 |
| NCI-UMC-11- Lung carcinoid | 1.2 |
| LX-1- Small cell lung cancer | 0.0 |
| Colo-205- Colon cancer | 0.0 |
| KM12- Colon cancer | 0.0 |
| KM20L2- Colon cancer | 0.0 |
| NCI-H716- Colon cancer | 100.0 |
| SW-48- Colon adenocarcinoma | 0.0 |
| SW1116- Colon adenocarcinoma | 0.0 |
| LS 174T- Colon adenocarcinoma | 0.0 |
| SW-948- Colon adenocarcinoma | 0.0 |
| SW-480- Colon adenocarcinoma | 1.1 |
| NCI-SNU-5- Gastric carcinoma | 0.0 |
| KATO III- Gastric carcinoma | 0.0 |
| NCI-SNU-16- Gastric carcinoma | 0.4 |
| NCI-SNU-1- Gastric carcinoma | 0.0 |
| RF-1- Gastric adenocarcinoma | 0.0 |
| RF-48- Gastric adenocarcinoma | 0.0 |
| MKN-45- Gastric carcinoma | 0.0 |
| NCI-N87- Gastric carcinoma | 0.0 |
| OVCAR-5- Ovarian carcinoma | 0.0 |
| RL95-2- Uterine carcinoma | 0.2 |
| HelaS3- Cervical adenocarcinoma | 0.0 |
| Ca Ski- Cervical epidermoid carcinoma | 0.0 |
| (metastasis) | |
| ES-2- Ovarian clear cell carcinoma | 0.3 |
| Ramos- Stimulated with PMA/ionomycin 6 h | 0.0 |
| Ramos- Stimulated with PMA/ionomycin 14 h | 0.0 |
| MEG-01- Chronic myelogenous leukemia | 0.6 |
| (megokaryoblast) | |
| Raji- Burkitt's lymphoma | 0.0 |
| Daudi- Burkitt's lymphoma | 0.0 |
| U266- B-cell plasmacytoma | 0.0 |
| CA46- Burkitt's lymphoma | 0.0 |
| RL- non-Hodgkin's B-cell lymphoma | 0.0 |
| JM1- pre-B-cell lymphoma | 0.0 |
| Jurkat- T cell leukemia | 0.2 |
| TF-1- Erythroleukemia | 0.0 |
| HUT 78- T-cell lymphoma | 0.0 |
| U937- Histiocytic lymphoma | 0.0 |
| KU-812- Myelogenous leukemia | 0.0 |
| 769-P- Clear cell renal carcinoma | 0.0 |
| Caki-2- Clear cell renal carcinoma | 32.8 |
| SW 839- Clear cell renal carcinoma | 0.2 |
| Rhabdoid kidney tumor | 0.0 |
| Hs766T- Pancreatic carcinoma (LN | 0.0 |
| metastasis) | |
| CAPAN-1- Pancreatic adenocarcinoma (liver | 0.0 |
| metastasis) | |
| SU86.86- Pancreatic carcinoma (liver | 1.5 |
| metastasis) | |
| BxPC-3- Pancreatic adenocarcinoma | 0.0 |
| HPAC- Pancreatic adenocarcinoma | 0.3 |
| MIA PaCa-2- Pancreatic carcinoma | 0.0 |
| CFPAC-1- Pancreatic ductal adenocarcinoma | 0.0 |
| PANC-1- Pancreatic epithelioid ductal | 0.8 |
| carcinoma | |
| T24- Bladder carcinma (transitional cell) | 0.0 |
| 5637- Bladder carcinoma | 0.0 |
| HT-1197- Bladder carcinoma | 0.0 |
| UM-UC-3- Bladder carcinma (transitional cell) | 0.0 |
| A204- Rhabdomyosarcoma | 0.0 |
| HT-1080- Fibrosarcoma | 0.3 |
| MG-63- Osteosarcoma | 0.0 |
| SK-LMS-1- Leiomyosarcoma (vulva) | 4.6 |
| SJRH30- Rhabdomyosarcoma (met to bone marrow) | 0.2 |
| A431- Epidermoid carcinoma | 0.0 |
| WM266-4- Melanoma | 0.0 |
| DU 145- Prostate carcinoma (brain | 0.0 |
| metastasis) | |
| MDA-MB-468- Breast adenocarcinoma | 1.3 |
| SCC-4- Squamous cell carcinoma of tongue | 0.0 |
| SCC-9- Squamous cell carcinoma of tongue | 0.0 |
| SCC-15- Squamous cell carcinoma of tongue | 0.0 |
| CAL 27- Squamous cell carcinoma of tongue | 0.0 |

Column A - Rel. Exp. (%) Ag3024, Run 164886426

TABLE ARF
Panel 4D

| Tissue Name | A |
|---|---|
| Secondary Th1 act | 0.0 |
| Secondary Th2 act | 0.0 |
| Secondary Tr1 act | 0.0 |
| Secondary Th1 rest | 0.0 |
| Secondary Th2 rest | 4.4 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 4.7 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 5.3 |
| HUVEC none | 24.7 |
| HUVEC starved | 11.8 |
| HUVEC IL-1beta | 0.0 |
| HUVEC IFN gamma | 31.4 |
| HUVEC TNF alpha + IFN gamma | 14.9 |
| HUVEC TNF alpha + IL4 | 8.8 |
| HUVEC IL-11 | 11.4 |
| Lung Microvascular EC none | 10.1 |
| Lung Microvascular EC TNFalpha + IL-1beta | 8.7 |
| Microvascular Dermal EC none | 11.3 |
| Microsvasular Dermal EC TNFalpha + IL-1beta | 3.0 |
| Bronchial epithelium TNFalpha + IL1beta | 5.3 |
| Small airway epithelium none | 3.7 |
| Small airway epithelium TNFalpha + IL-1beta | 66.0 |
| Coronery artery SMC rest | 21.0 |
| Coronery artery SMC TNFalpha + IL-1beta | 10.4 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL-1beta | 0.0 |
| Liver cirrhosis | 17.7 |
| Lupus kidney | 5.6 |
| NCI-H292 none | 3.2 |
| NCI-H292 IL-4 | 0.0 |
| NCI-H292 IL-9 | 10.4 |
| NCI-H292 IL-13 | 0.0 |
| NCI-H292 IFN gamma | 0.0 |
| HPAEC none | 6.3 |
| HPAEC TNF alpha + IL-1 beta | 3.2 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 4.1 |
| Dermal fibroblast IFN gamma | 6.1 |
| Dermal fibroblast IL-4 | 8.7 |
| IBD Colitis 2 | 0.0 |
| IBD Crohn's | 7.7 |
| Colon | 24.1 |
| Lung | 14.5 |
| Thymus | 100.0 |
| Kidney | 0.0 |

Column A - Rel. Exp. (%) Ag3024, Run 162427416

AI_comprehensive panel_v1.0 Summary: Ag3024 This gene was found at low but significant levels in lung tissue from COPD, emphysema and asthma patients. This expression was consistent with panel 4D which showed expression in small airway epithelium. Therefore, this gene is useful as a marker or a target for lung inflammatory diseases.

Panel 1.3D Summary: Ag3024 Expression of this gene, a heparin sulfate proteoglycan homolog, was highly brain preferential and plays a role for this gene product in CNS processes. Heparin sulfate proteoglycans (HSPGs) are a component of amyloid plaques in Alzheimer's disease. The interaction of apoE with HSPGs has also been implicated in the pathogenesis of Alzheimer's disease and plays a role in neuronal repair. apoE has an HSPG-binding site highly complementary to heparan sulfates rich in N- and O-sulfo groups. (Libeu C P, E. J Biol Chem 2001 October 19;276(42):39138-44). Enzymes that influence the structure of HSPGs, such as the protein product of this gene, will influence protein agregation and the functional processes underlying Alzheimer's disease. Agents that target and modulate the activity of this gene or gene product are effective in the treatment of neurodegenerative diseases including Alzheimer's disease. This gene was also expressed in breast and brain cancer cell lines at low but significant levels. The expression of this gene is of use as a marker for breast and brain cancer. Therapeutic inhibition of the activity of the product of this gene, through the use of antibodies or small molecule drugs, is useful in the therapy of brain and breast cancer.

Panel 2D Summary: Ag3024 This gene was expressed at low but significant levels in most of the samples on this panel, with highest expression in a kidney cancer sample (CT=30.6). Significant levels of expression were also seen in samples derived from breast and gastric cancer samples. Expression of this gene is of use as a marker for breast and gastric cancer. Therapeutic inhibition of the activity of the product of this gene or gene product, through the use of antibodies or small molecule drugs, is of use in the therapy of breast and gastric cancer.

Panel 3D Summary: Ag3024 This gene was expressed at low but significant levels in cell lines from a renal carcinoma, colon cancer, glioblastoma and three lung cancer lines. Thus, this gene is useful as a marker as well as a target for inhibition of these cancers.

Panel 4D Summary: Ag3024 This gene, a heparin Sulfate 6-Sulfotransferase 3 homolog, was expressed at low but significant levels in thymus and small airway epithelium treated with TNFalpha+IL-1beta (CTs=34). The gene product is of use as a marker for thymus or activated small airway epithelium.

AS. CG56904-01: Secreted LRR (Leucine-rich Repeat) Protein

Expression of gene CG56904-01 was assessed using the primer-probe sets Ag3048, and Ag6717, described in Tables ASA and ASB. Results of the RTQ-PCR runs are shown in Tables ASC, ASD, ASE, ASF, and ASG.

TABLE ASA
Probe Name Ag3048

| | | | | SEQ |
|---|---|---|---|---|
| | | | Start | ID |
| Primers | Sequences | Length | Position | No |
| Forward | 5′-cacaaccagctgacagacagt-3′ | 21 | 490 | 1504 |
| Probe | TET-5′-ccaccaccttcagcaagctgcatag-3′- | 25 | 521 | 1505 |
| Reverse | 5′-gggagagatccaggtattcaag-3′ | 22 | 547 | 1506 |

TABLE ASB
Probe Name Ag6717

| | | | | SEQ |
|---|---|---|---|---|
| | | | Start | ID |
| Primers | Sequences | Length | Position | No |
| Forward | 5′-gctcacaacaagaacaatctc-3 | 21 | 406 | 1507 |
| Probe | TET-5′-aaggtgccccgaggagc-3′-TAMRA | 17 | 433 | 1508 |
| Reverse | 5′-cacggagttgagtctgg-3′ | 17 | 459 | 1509 |

TABLE ASC
AI_comprehensive panel_v1.0

| Tissue Name | A |
|---|---|
| 110967 COPD-F | 0.0 |
| 110980 COPD-F | 0.7 |
| 110968 COPD-M | 1.0 |
| 110977 COPD-M | 0.8 |
| 110989 Emphysema-F | 0.8 |
| 110992 Emphysema-F | 6.3 |
| 110993 Emphysema-F | 0.3 |
| 110994 Emphysema-F | 0.0 |
| 110995 Emphysema-F | 12.7 |
| 110996 Emphysema-F | 6.6 |
| 110997 Asthma-M | 2.6 |
| 111001 Asthma-F | 0.4 |
| 111002 Asthma-F | 1.9 |
| 111003 Atopic Asthma-F | 1.6 |
| 111004 Atopic Asthma-F | 5.2 |
| 111005 Atopic Asthma-F | 2.8 |
| 111006 Atopic Asthma-F | 0.3 |
| 111417 Allergy-M | 0.4 |
| 112347 Allergy-M | 0.0 |
| 112349 Normal Lung-F | 0.0 |
| 112357 Normal Lung-F | 8.5 |
| 112354 Normal Lung-M | 0.9 |
| 112374 Crohns-F | 4.0 |
| 112389 Match Control Crohns-F | 17.3 |
| 112375 Crohns-F | 4.2 |
| 112732 Match Control Crohns-F | 1.1 |
| 112725 Crohns-M | 0.0 |
| 112387 Match Control Crohns-M | 3.6 |
| 112378 Crohns-M | 0.0 |
| 112390 Match Control Crohns-M | 0.5 |
| 112726 Crohns-M | 0.0 |
| 112731 Match Control Crohns-M | 1.4 |
| 112380 Ulcer Col-F | 0.9 |
| 112734 Match Control Ulcer Col-F | 4.9 |
| 112384 Ulcer Col-F | 6.8 |
| 112737 Match Control Ulcer Col-F | 0.7 |
| 112386 Ulcer Col-F | 7.0 |
| 112738 Match Control Ulcer Col-F | 5.1 |
| 112381 Ulcer Col-M | 0.0 |
| 112735 Match Control Ulcer Col-M | 0.0 |
| 112382 Ulcer Col-M | 11.8 |
| 112394 Match Control Ulcer Col-M | 1.6 |
| 112383 Ulcer Col-M | 30.8 |
| 112736 Match Control Ulcer Col-M | 6.3 |
| 112423 Psoriasis-F | 0.7 |
| 112427 Match Control Psoriasis-F | 1.4 |
| 112418 Psoriasis-M | 0.6 |
| 112723 Match Control Psoriasis-M | 4.8 |
| 112419 Psoriasis-M | 0.9 |
| 112424 Match Control Psoriasis-M | 0.9 |
| 112420 Psoriasis-M | 6.4 |
| 112425 Match Control Psoriasis-M | 0.4 |
| 104689 (MF) OA Bone-Backus | 54.7 |
| 104690 (MF) Adj “Normal” Bone-Backus | 23.3 |
| 104691 (MF) OA Synovium-Backus | 5.3 |
| 104692 (BA) OA Cartilage-Backus | 0.0 |
| 104694 (BA) OA Bone-Backus | 100.0 |
| 104695 (BA) Adj “Normal” Bone-Backus | 80.1 |
| 104696 (BA) OA Synovium-Backus | 27.4 |
| 104700 (SS) OA Bone-Backus | 21.6 |
| 104701 (SS) Adj “Normal” Bone-Backus | 90.8 |
| 104702 (SS) OA Synovium-Backus | 76.3 |
| 117093 OA Cartilage Rep7 | 7.9 |
| 112672 OA Bone5 | 0.7 |
| 112673 OA Synovium5 | 0.4 |
| 112674 OA Synovial Fluid cells5 | 0.0 |
| 117100 OA Cartilage Rep14 | 0.0 |
| 112756 OA Bone9 | 8.6 |
| 112757 OA Synovium9 | 0.7 |
| 112758 OA Synovial Fluid Cells9 | 3.8 |
| 117125 RA Cartilage Rep2 | 0.5 |
| 113492 Bone2 RA | 3.7 |
| 113493 Synovium2 RA | 1.2 |
| 113494 Syn Fluid Cells RA | 1.9 |
| 113499 Cartilage4 RA | 1.4 |
| 113500 Bone4 RA | 3.1 |
| 113501 Synovium4 RA | 2.4 |
| 113502 Syn Fluid Cells4 RA | 1.2 |
| 113495 Cartilage3 RA | 2.0 |
| 113496 Bone3 RA | 2.0 |
| 113497 Synovium3 RA | 1.7 |
| 113498 Syn Fluid Cells3 RA | 2.5 |
| 117106 Normal Cartilage Rep20 | 0.4 |
| 113663 Bone3 Normal | 0.0 |
| 113664 Synovium3 Normal | 0.0 |
| 113665 Syn Fluid Cells3 Normal | 0.0 |
| 117107 Normal Cartilage Rep22 | 0.0 |
| 113667 Bone4 Normal | 0.4 |
| 113668 Synovium4 Normal | 0.5 |
| 113669 Syn Fluid Cells4 Normal | 1.0 |

Column A - Rel. Exp. (%) Ag6717, Run 283839606

TABLE ASD
Panel 1.3D

| Tissue Name | A |
|---|---|
| Liver adenocarcinoma | 2.0 |
| Pancreas | 0.0 |
| Pancreatic ca. CAPAN 2 | 0.3 |
| Adrenal gland | 0.0 |
| Thyroid | 0.5 |
| Salivary gland | 0.6 |
| Pituitary gland | 0.5 |
| Brain (fetal) | 0.9 |
| Brain (whole) | 2.4 |
| Brain (amygdala) | 2.9 |
| Brain (cerebellum) | 7.0 |
| Brain (hippocampus) | 2.1 |
| Brain (substantia nigra) | 4.5 |
| Brain (thalamus) | 4.6 |
| Cerebral Cortex | 1.0 |
| Spinal cord | 7.6 |
| glio/astro U87-MG | 2.8 |
| glio/astro U-118-MG | 5.9 |
| astrocytoma SW1783 | 2.7 |
| neuro*; met SK-N-AS | 1.1 |
| astrocytoma SF-539 | 100.0 |
| astrocytoma SNB-75 | 41.2 |
| glioma SNB-19 | 0.4 |
| glioma U251 | 6.7 |
| glioma SF-295 | 7.3 |
| Heart (fetal) | 5.1 |
| Heart | 1.3 |
| Skeletal muscle (fetal) | 4.7 |
| Skeletal muscle | 2.6 |
| Bone marrow | 0.9 |
| Thymus | 0.9 |
| Spleen | 0.3 |
| Lymph node | 0.0 |
| Colorectal | 2.9 |
| Stomach | 0.8 |
| Small intestine | 1.0 |
| Colon ca. SW480 | 0.8 |
| Colon ca.* SW620(SW480 met) | 1.9 |
| Colon ca. HT29 | 0.3 |
| Colon ca. HCT-116 | 0.0 |
| Colon ca. CaCo-2 | 0.2 |
| Colon ca. tissue(ODO3866) | 13.2 |
| Colon ca. HCC-2998 | 4.1 |
| Gastric ca.* (liver met) NCI-N87 | 1.5 |
| Bladder | 3.0 |
| Trachea | 3.7 |
| Kidney | 0.0 |
| Kidney (fetal) | 0.6 |
| Renal ca. 786-0 | 3.8 |
| Renal ca. A498 | 17.7 |
| Renal ca. RXF 393 | 12.5 |
| Renal ca. ACHN | 0.1 |
| Renal ca. UO-31 | 1.1 |
| Renal ca. TK-10 | 0.5 |
| Liver | 0.0 |
| Liver (fetal) | 0.0 |
| Liver ca. (hepatoblast) HepG2 | 0.2 |
| Lung | 1.4 |
| Lung (fetal) | 7.1 |
| Lung ca. (small cell) LX-1 | 0.2 |
| Lung ca. (small cell) NCI-H69 | 0.0 |
| Lung ca. (s. cell var.) SHP-77 | 0.9 |
| Lung ca. (large cell)NCI-H460 | 0.0 |
| Lung ca. (non-sm. cell) A549 | 0.0 |
| Lung ca. (non-s. cell) NCI-H23 | 0.4 |
| Lung ca. (non-s. cell) HOP-62 | 41.2 |
| Lung ca. (non-s. cl) NCI-H522 | 1.6 |
| Lung ca. (squam.) SW 900 | 0.0 |
| Lung ca. (squam.) NCI-H596 | 0.0 |
| Mammary gland | 15.9 |
| Breast ca.* (pl. ef) MCF-7 | 3.5 |
| Breast ca.* (pl. ef) MDA-MB-231 | 2.0 |
| Breast ca.* (pl. ef) T47D | 0.3 |
| Breast ca. BT-549 | 0.0 |
| Breast ca. MDA-N | 0.3 |
| Ovary | 3.8 |
| Ovarian ca. OVCAR-3 | 0.2 |
| Ovarian ca. OVCAR-4 | 0.2 |
| Ovarian ca. OVCAR-5 | 4.3 |
| Ovarian ca. OVCAR-8 | 0.8 |
| Ovarian ca. IGROV-1 | 0.0 |
| Ovarian ca.* (ascites) SK-OV-3 | 1.5 |
| Uterus | 0.6 |
| Placenta | 0.9 |
| Prostate | 3.0 |
| Prostate ca.* (bone met)PC-3 | 1.7 |
| Testis | 3.9 |
| Melanoma Hs688(A).T | 32.8 |
| Melanoma* (met) Hs688(B).T | 31.4 |
| Melanoma UACC-62 | 1.8 |
| Melanoma M14 | 0.0 |
| Melanoma LOX IMVI | 0.0 |
| Melanoma* (met) SK-MEL-5 | 0.0 |
| Adipose | 4.3 |

Column A - Rel. Exp. (%) Ag3048, Run 168017062

TABLE ASE
Panel 2D

| Tissue Name | A |
|---|---|
| Normal Colon | 4.7 |
| CC Well to Mod Diff (ODO3866) | 12.0 |
| CC Margin (ODO3866) | 1.5 |
| CC Gr.2 rectosigmoid (ODO3868) | 10.1 |
| CC Margin (ODO3868) | 0.8 |
| CC Mod Diff (ODO3920) | 8.4 |
| CC Margin (ODO3920) | 2.6 |
| CC Gr.2 ascend colon (ODO3921) | 13.6 |
| CC Margin (ODO3921) | 2.1 |
| CC from Partial Hepatectomy (ODO4309) Mets | 3.1 |
| Liver Margin (ODO4309) | 0.1 |
| Colon mets to lung (OD04451-01) | 3.3 |
| Lung Margin (OD04451-02) | 1.1 |
| Normal Prostate 6546-1 | 4.1 |
| Prostate Cancer (OD04410) | 19.8 |
| Prostate Margin (OD04410) | 4.5 |
| Prostate Cancer (OD04720-01) | 9.7 |
| Prostate Margin (OD04720-02) | 7.5 |
| Normal Lung 061010 | 3.3 |
| Lung Met to Muscle (ODO4286) | 24.5 |
| Muscle Margin (ODO4286) | 1.7 |
| Lung Malignant Cancer (OD03126) | 11.6 |
| Lung Margin (OD03126) | 3.1 |
| Lung Cancer (OD04404) | 22.2 |
| Lung Margin (OD04404) | 7.2 |
| Lung Cancer (OD04565) | 36.1 |
| Lung Margin (OD04565) | 2.0 |
| Lung Cancer (OD04237-01) | 12.6 |
| Lung Margin (OD04237-02) | 6.7 |
| Ocular Mel Met to Liver (ODO4310) | 0.0 |
| Liver Margin (ODO4310) | 0.4 |
| Melanoma Mets to Lung (OD04321) | 0.0 |
| Lung Margin (OD04321) | 1.7 |
| Normal Kidney | 0.8 |
| Kidney Ca, Nuclear grade 2 (OD04338) | 0.3 |
| Kidney Margin (OD04338) | 1.5 |
| Kidney Ca Nuclear grade 1/2 (OD04339) | 0.2 |
| Kidney Margin (OD04339) | 0.9 |
| Kidney Ca, Clear cell type (OD04340) | 0.4 |
| Kidney Margin (OD04340) | 1.0 |
| Kidney Ca, Nuclear grade 3 (OD04348) | 10.3 |
| Kidney Margin (OD04348) | 0.4 |
| Kidney Cancer (OD04622-01) | 34.4 |
| Kidney Margin (OD04622-03) | 0.2 |
| Kidney Cancer (OD04450-01) | 0.0 |
| Kidney Margin (OD04450-03) | 0.2 |
| Kidney Cancer 8120607 | 9.0 |
| Kidney Margin 8120608 | 0.5 |
| Kidney Cancer 8120613 | 0.1 |
| Kidney Margin 8120614 | 0.4 |
| Kidney Cancer 9010320 | 13.1 |
| Kidney Margin 9010321 | 0.9 |
| Normal Uterus | 0.5 |
| Uterus Cancer 064011 | 1.0 |
| Normal Thyroid | 3.3 |
| Thyroid Cancer 064010 | 0.7 |
| Thyroid Cancer A302152 | 4.5 |
| Thyroid Margin A302153 | 0.8 |
| Normal Breast | 20.0 |
| Breast Cancer (OD04566) | 6.5 |
| Breast Cancer (OD04590-01) | 10.4 |
| Breast Cancer Mets (OD04590-03) | 11.4 |
| Breast Cancer Metastasis (OD04655-05) | 1.0 |
| Breast Cancer 064006 | 23.2 |
| Breast Cancer 1024 | 25.7 |
| Breast Cancer 9100266 | 16.4 |
| Breast Margin 9100265 | 24.0 |
| Breast Cancer A209073 | 32.3 |
| Breast Margin A209073 | 33.2 |
| Normal Liver | 0.0 |
| Liver Cancer 064003 | 0.0 |
| Liver Cancer 1025 | 0.0 |
| Liver Cancer 1026 | 4.2 |
| Liver Cancer 6004-T | 0.0 |
| Liver Tissue 6004-N | 2.9 |
| Liver Cancer 6005-T | 2.2 |
| Liver Tissue 6005-N | 0.0 |
| Normal Bladder | 23.8 |
| Bladder Cancer 1023 | 11.8 |
| Bladder Cancer A302173 | 7.2 |
| Bladder Cancer (OD04718-01) | 12.7 |
| Bladder Normal Adjacent (OD04718-03) | 4.9 |
| Normal Ovary | 3.5 |
| Ovarian Cancer 064008 | 100.0 |
| Ovarian Cancer (OD04768-07) | 2.5 |
| Ovary Margin (OD04768-08) | 8.1 |
| Normal Stomach | 1.5 |
| Gastric Cancer 9060358 | 0.5 |
| Stomach Margin 9060359 | 0.8 |
| Gastric Cancer 9060395 | 7.5 |
| Stomach Margin 9060394 | 1.9 |
| Gastric Cancer 9060397 | 27.0 |
| Stomach Margin 9060396 | 1.5 |
| Gastric Cancer 064005 | 3.3 |

Column A - Rel. Exp. (%) Ag3048, Run 170858352

TABLE ASF
Panel 3D

| Tissue Name | A |
|---|---|
| Daoy- Medulloblastoma | 6.5 |
| TE671- Medulloblastoma | 0.0 |
| D283 Med- Medulloblastoma | 0.0 |
| PFSK-1- Primitive Neuroectodermal | 18.0 |
| XF-498- CNS | 1.4 |
| SNB-78- Glioma | 100.0 |
| SF-268- Glioblastoma | 0.0 |
| T98G- Glioblastoma | 24.1 |
| SK-N-SH- Neuroblastoma (metastasis) | 32.3 |
| SF-295- Glioblastoma | 2.0 |
| Cerebellum | 3.0 |
| Cerebellum | 4.2 |
| NCI-H292- Mucoepidermoid lung carcinoma | 38.4 |
| DMS-114- Small cell lung cancer | 1.7 |
| DMS-79- Small cell lung cancer | 3.0 |
| NCI-H146- Small cell lung cancer | 3.3 |
| NCI-H526- Small cell lung cancer | 0.0 |
| NCI-N417- Small cell lung cancer | 0.0 |
| NCI-H82- Small cell lung cancer | 0.0 |
| NCI-H157- Squamous cell lung cancer | 1.4 |
| (metastasis) | |
| NCI-H1155- Large cell lung cancer | 1.8 |
| NCI-H1299- Large cell lung cancer | 2.8 |
| NCI-H727- Lung carcinoid | 0.0 |
| NCI-UMC-11- Lung carcinoid | 0.0 |
| LX-1- Small cell lung cancer | 0.0 |
| Colo-205- Colon cancer | 7.3 |
| KM12- Colon cancer | 1.6 |
| KM20L2- Colon cancer | 0.0 |
| NCI-H716- Colon cancer | 0.0 |
| SW-48- Colon adenocarcinoma | 0.6 |
| SW1116- Colon adenocarcinoma | 0.8 |
| LS 174T- Colon adenocarcinoma | 2.5 |
| SW-948- Colon adenocarcinoma | 0.0 |
| SW-480- Colon adenocarcinoma | 2.7 |
| NCI-SNU-5- Gastric carcinoma | 1.6 |
| KATO III- Gastric carcinoma | 1.8 |
| NCI-SNU-16- Gastric carcinoma | 1.4 |
| NCI-SNU-1- Gastric carcinoma | 0.0 |
| RF-1- Gastric adenocarcinoma | 0.0 |
| RF-48- Gastric adenocarcinoma | 0.9 |
| MKN-45- Gastric carcinoma | 5.6 |
| NCI-N87- Gastric carcinoma | 2.3 |
| OVCAR-5- Ovarian carcinoma | 4.8 |
| RL95-2- Uterine carcinoma | 0.0 |
| HelaS3- Cervical adenocarcinoma | 0.0 |
| Ca Ski- Cervical epidermoid carcinoma | 0.0 |
| (metastasis) | |
| ES-2- Ovarian clear cell carcinoma | 0.0 |
| Ramos- Stimulated with PMA/ionomycin 6 h | 0.0 |
| Ramos- Stimulated with PMA/ionomycin 14 h | 0.0 |
| MEG-01- Chronic myelogenous leukemia | 2.6 |
| (megokaryoblast) | |
| Raji- Burkitt's lymphoma | 0.0 |
| Daudi- Burkitt's lymphoma | 0.0 |
| U266- B-cell plasmacytoma | 0.0 |
| CA46- Burkitt's lymphoma | 0.0 |
| RL- non-Hodgkin's B-cell lymphoma | 0.0 |
| JM1- pre-B-cell lymphoma | 0.0 |
| Jurkat- T cell leukemia | 0.0 |
| TF-1- Erythroleukemia | 2.3 |
| HUT 78- T-cell lymphoma | 0.0 |
| U937- Histiocytic lymphoma | 0.7 |
| KU-812- Myelogenous leukemia | 0.0 |
| 769-P- Clear cell renal carcinoma | 0.0 |
| Caki-2- Clear cell renal carcinoma | 2.5 |
| SW 839- Clear cell renal carcinoma | 0.0 |
| Rhabdoid kidney tumor | 2.3 |
| Hs766T- Pancreatic carcinoma (LN | 1.5 |
| metastasis) | |
| CAPAN-1- Pancreatic adenocarcinoma (liver | 0.0 |
| metastasis) | |
| SU86.86- Pancreatic carcinoma (liver | 0.8 |
| metastasis) | |
| BxPC-3- Pancreatic adenocarcinoma | 0.7 |
| HPAC- Pancreatic adenocarcinoma | 0.0 |
| MIA PaCa-2- Pancreatic carcinoma | 0.7 |
| CFPAC-1- Pancreatic ductal adenocarcinoma | 1.7 |
| PANC-1- Pancreatic epithelioid ductal | 6.7 |
| carcinoma | |
| T24- Bladder carcinma (transitional cell) | 6.1 |
| 5637- Bladder carcinoma | 2.1 |
| HT-1197- Bladder carcinoma | 1.7 |
| UM-UC-3- Bladder carcinma (transitional cell) | 0.7 |
| A204- Rhabdomyosarcoma | 5.4 |
| HT-1080- Fibrosarcoma | 11.7 |
| MG-63- Osteosarcoma | 0.0 |
| SK-LMS-1- Leiomyosarcoma (vulva) | 7.5 |
| SJRH30- Rhabdomyosarcoma (met to bone | 0.0 |
| marrow) | |
| A431- Epidermoid carcinoma | 5.1 |
| WM266-4- Melanoma | 16.6 |
| DU 145- Prostate carcinoma (brain | 0.0 |
| metastasis) | |
| MDA-MB-468- Breast adenocarcinoma | 2.5 |
| SCC-4- Squamous cell carcinoma of tongue | 0.0 |
| SCC-9- Squamous cell carcinoma of tongue | 0.0 |
| SCC-15- Squamous cell carcinoma of tongue | 0.6 |
| CAL 27- Squamous cell carcinoma of tongue | 0.5 |

Column A - Rel. Exp. (%) Ag3048, Run 172133336

TABLE ASG
Panel 4D

| | |
|---|---|
| Monocytes LPS | 0.0 |
| Macrophages rest | 1.9 |
| Macrophages LPS | 1.3 |
| HUVEC none | 0.0 |
| HUVEC starved | 2.9 |
| Colon | 2.0 |
| Lung | 36.1 |
| Thymus | 0.6 |
| Kidney | 1.7 |

AI_comprehensive panel_v1.0 Summary: Ag6717 Prominent expression was seen in bone and synovium samples from osteoarthritis patients, with highest expression in an OA bone sample (CT=31). Thus, targeting this gene or its protein product is useful in the treatment of osteoarthritis.

Panel 1.3D Summary: Ag3048 The expression of this gene was highest in a sample derived from a brain cancer cell line (SF-539) (CT=29.4). There was substantial expression associated with samples derived from another brain cancer cell line, two melanoma cell lines and a lung cancer cell line. The expression of this gene is useful as a marker to distinguish SF-539 cells from other samples in the panel. Therapeutic modulation of this gene, through the use of small molecule drugs, protein therapeuitcs or antibodies is of benefit in treatment of brain or lung cancer or melanoma.

This gene, a leucine-rich repeat homolog, was expressed at low levels in the CNS. The leucine-rich repeat region proteins have been implicated in axonal guidance. This gene has therapeutic utility in the treatment of any CNS disorder involving neuronal loss, specfically by guiding/enhancing compensatory synaptogenesis and fiber outgrowth, including such clinical conditions as Alzheimer's, Parkinson's, or Huntington's diseases, stroke, head and spinal cord trauma, vascular dementia or spinocerebellar ataxia.

Panel 2D Summary: Ag3048 The expression of this gene was highest in a sample derived from an ovarian cancer (CT=29). There was substantial expression associated with lung cancer, prostate cancer and colon cancer samples. Of note is the differential expression in the lung, colon and prostate cancer samples compared to their respective normal adjacent tissue. The expression of this gene is useful as a marker to distinguish this ovarian cancer sample from other samples in the panel. The expression of this gene or its protein product is useful as a marker to distinguish colon, prostate or lung cancer samples from their normal adjacent tissue. Therapeutic modulation of this gene or its protein product, through the use of small molecule drugs, protein therapeutics or antibodies is benefical in the treatment of ovarian, lung, prostate or colon cancer.

Panel 3D Sumunary: Ag3048 The expression of this gene was highest in a sample derived from a brain cancer cell line (SNB-78) (CT=30.2). There was substantial expression associated with other brain cancer cell line samples and a lung cancer cell line sample. The expression of this gene is useful as a marker to distinguish SNB-78 cells from other samples in the panel. Therapeutic modulation of this gene or its protein product, through the use of small molecule drugs, protein therapeutics or antibodies is useful in the treatment of brain or lung cancer.

Panel 4D Summary: Ag3048 This gene, a secreted leucine-rich repeat (LRR) protein, was expressed selectively at moderate levels (CT range 29-31) in several resting and cytokine-activated epithelial and connective tissue cells of lung and skin. This gene product is useful as a therapeutic protein as well as a target for therapeutic antibodies and small molecules, for the reduction or elimination of the symptoms in patients with chronic obstructive pulmonary disease, asthma, emphysema, or psoriasis.

AT. CG56904-04: LRR Protein

Expression of gene CG56904-04 was assessed using the primer-probe set Ag3048, described in Table ATA. Results of the RTQ-PCR runs are shown in Tables ATB, ATC, ATD and ATE.

TABLE ATA
Probe Name Ag3048

| | | | | SEQ |
|---|---|---|---|---|
| | | | Start | ID |
| Primers | Sequences | Length | Position | No |
| Forward | 5′-cacaaccagctgacagacagt-3′ | 21 | 724 | 1510 |
| Probe | TET-5′-ccaccaccttcagcaagctgcatag-3′- | 25 | 755 | 1511 |
| | TAMRA | | | |
| Reverse | 5′-gggagagatccaggtattcaag-3′ | 22 | 781 | 1512 |

TABLE ATB
Panel 1.3D

| Tissue Name | A |
|---|---|
| Liver adenocarcinoma | 2.0 |
| Pancreas | 0.0 |
| Pancreatic ca. CAPAN 2 | 0.3 |
| Adrenal gland | 0.0 |
| Thyroid | 0.5 |
| Salivary gland | 0.6 |
| Pituitary gland | 0.5 |
| Brain (fetal) | 0.9 |
| Brain (whole) | 2.4 |
| Brain (amygdala) | 2.9 |
| Brain (cerebellum) | 7.0 |
| Brain (hippocampus) | 2.1 |
| Brain (substantia nigra) | 4.5 |
| Brain (thalamus) | 4.6 |
| Cerebral Cortex | 1.0 |
| Spinal cord | 7.6 |
| glio/astro U87-MG | 2.8 |
| glio/astro U-118-MG | 5.9 |
| astrocytoma SW1783 | 2.7 |
| neuro*; met SK-N-AS | 1.1 |
| astrocytoma SF-539 | 100.0 |
| astrocytoma SNB-75 | 41.2 |
| glioma SNB-19 | 0.4 |
| glioma U251 | 6.7 |
| glioma SF-295 | 7.3 |
| Heart (fetal) | 5.1 |
| Heart | 1.3 |
| Skeletal muscle (fetal) | 4.7 |
| Skeletal muscle | 2.6 |
| Bone marrow | 0.9 |
| Thymus | 0.9 |
| Spleen | 0.3 |
| Lymph node | 0.0 |
| Colorectal | 2.9 |
| Stomach | 0.8 |
| Small intestine | 1.0 |
| Colon ca. SW480 | 0.8 |
| Colon ca.* SW620(SW480 met) | 1.9 |
| Colon ca. HT29 | 0.3 |
| Colon ca. HCT-116 | 0.0 |
| Colon ca. CaCo-2 | 0.2 |
| Colon ca. tissue(ODO3866) | 13.2 |
| Colon ca. HCC-2998 | 4.1 |
| Gastric ca.* (liver met) NCI-N87 | 1.5 |
| Bladder | 3.0 |
| Trachea | 3.7 |
| Kidney | 0.0 |
| Kidney (fetal) | 0.6 |
| Renal ca. 786-0 | 3.8 |
| Renal ca. A498 | 17.7 |
| Renal ca. RXF 393 | 12.5 |
| Renal ca. ACHN | 0.1 |
| Renal ca. UO-31 | 1.1 |
| Renal ca. TK-10 | 0.5 |
| Liver | 0.0 |
| Liver (fetal) | 0.0 |
| Liver ca. (hepatoblast) HepG2 | 0.2 |
| Lung | 1.4 |
| Lung (fetal) | 7.1 |
| Lung ca. (small cell) LX-1 | 0.2 |
| Lung ca. (small cell) NCI-H69 | 0.0 |
| Lung ca. (s. cell var.) SHP-77 | 0.9 |
| Lung ca. (large cell)NCI-H460 | 0.0 |
| Lung ca. (non-sm. cell) A549 | 0.0 |
| Lung ca. (non-s. cell) NCI-H23 | 0.4 |
| Lung ca. (non-s. cell) HOP-62 | 41.2 |
| Lung ca. (non-s. cl) NCI-H522 | 1.6 |
| Lung ca. (squam.) SW 900 | 0.0 |
| Lung ca. (squam.) NCI-H596 | 0.0 |
| Mammary gland | 15.9 |
| Breast ca.* (pl. ef) MCF-7 | 3.5 |
| Breast ca.* (pl. ef) MDA-MB-231 | 2.0 |
| Breast ca.* (pl. ef) T47D | 0.3 |
| Breast ca. BT-549 | 0.0 |
| Breast ca. MDA-N | 0.3 |
| Ovary | 3.8 |
| Ovarian ca. OVCAR-3 | 0.2 |
| Ovarian ca. OVCAR-4 | 0.2 |
| Ovarian ca. OVCAR-5 | 4.3 |
| Ovarian ca. OVCAR-8 | 0.8 |
| Ovarian ca. IGROV-1 | 0.0 |
| Ovarian ca.* (ascites) SK-OV-3 | 1.5 |
| Uterus | 0.6 |
| Placenta | 0.9 |
| Prostate | 3.0 |
| Prostate ca.* (bone met)PC-3 | 1.7 |
| Testis | 3.9 |
| Melanoma Hs688(A).T | 32.8 |
| Melanoma* (met) Hs688(B).T | 31.4 |
| Melanoma UACC-62 | 1.8 |
| Melanoma M14 | 0.0 |
| Melanoma LOX IMVI | 0.0 |
| Melanoma* (met) SK-MEL-5 | 0.0 |
| Adipose | 4.3 |

Column A - Rel. Exp. (%) Ag3048, Run 168017062

TABLE ATC
Panel 2D

| Tissue Name | A |
|---|---|
| Normal Colon | 4.7 |
| CC Well to Mod Diff (ODO3866) | 12.0 |
| CC Margin (ODO3866) | 1.5 |
| CC Gr.2 rectosigmoid (ODO3868) | 10.1 |
| CC Margin (ODO3868) | 0.8 |
| CC Mod Diff (ODO3920) | 8.4 |
| CC Margin (ODO3920) | 2.6 |
| CC Gr.2 ascend colon (ODO3921) | 13.6 |
| CC Margin (ODO3921) | 2.1 |
| CC from Partial Hepatectomy (ODO4309) Mets | 3.1 |
| Liver Margin (ODO4309) | 0.1 |
| Colon mets to lung (OD04451-01) | 3.3 |
| Lung Margin (OD04451-02) | 1.1 |
| Normal Prostate 6546-1 | 4.1 |
| Prostate Cancer (OD04410) | 19.8 |
| Prostate Margin (OD04410) | 4.5 |
| Prostate Cancer (OD04720-01) | 9.7 |
| Prostate Margin (OD04720-02) | 7.5 |
| Normal Lung 061010 | 3.3 |
| Lung Met to Muscle (ODO4286) | 24.5 |
| Muscle Margin (ODO4286) | 1.7 |
| Lung Malignant Cancer (OD03126) | 11.6 |
| Lung Margin (OD03126) | 3.1 |
| Lung Cancer (OD04404) | 22.2 |
| Lung Margin (OD04404) | 7.2 |
| Lung Cancer (OD04565) | 36.1 |
| Lung Margin (OD04565) | 2.0 |
| Lung Cancer (OD04237-01) | 12.6 |
| Lung Margin (OD04237-02) | 6.7 |
| Ocular Mel Met to Liver (ODO4310) | 0.0 |
| Liver Margin (ODO4310) | 0.4 |
| Melanoma Mets to Lung (OD04321) | 0.0 |
| Lung Margin (OD04321) | 1.7 |
| Normal Kidney | 0.8 |
| Kidney Ca, Nuclear grade 2 (OD04338) | 0.3 |
| Kidney Margin (OD04338) | 1.5 |
| Kidney Ca Nuclear grade 1/2 (OD04339) | 0.2 |
| Kidney Margin (OD04339) | 0.9 |
| Kidney Ca, Clear cell type (OD04340) | 0.4 |
| Kidney Margin (OD04340) | 1.0 |
| Kidney Ca, Nuclear grade 3 (OD04348) | 10.3 |
| Kidney Margin (OD04348) | 0.4 |
| Kidney Cancer (OD04622-01) | 34.4 |
| Kidney Margin (OD04622-03) | 0.2 |
| Kidney Cancer (OD04450-01) | 0.0 |
| Kidney Margin (OD04450-03) | 0.2 |
| Kidney Cancer 8120607 | 9.0 |
| Kidney Margin 8120608 | 0.5 |
| Kidney Cancer 8120613 | 0.1 |
| Kidney Margin 8120614 | 0.4 |
| Kidney Cancer 9010320 | 13.1 |
| Kidney Margin 9010321 | 0.9 |
| Normal Uterus | 0.5 |
| Uterus Cancer 064011 | 1.0 |
| Normal Thyroid | 3.3 |
| Thyroid Cancer 064010 | 0.7 |
| Thyroid Cancer A302152 | 4.5 |
| Thyroid Margin A302153 | 0.8 |
| Normal Breast | 20.0 |
| Breast Cancer (OD04566) | 6.5 |
| Breast Cancer (OD04590-01) | 10.4 |
| Breast Cancer Mets (OD04590-03) | 11.4 |
| Breast Cancer Metastasis (OD04655-05) | 1.0 |
| Breast Cancer 064006 | 23.2 |
| Breast Cancer 1024 | 25.7 |
| Breast Cancer 9100266 | 16.4 |
| Breast Margin 9100265 | 24.0 |
| Breast Cancer A209073 | 32.3 |
| Breast Margin A209073 | 33.2 |
| Normal Liver | 0.0 |
| Liver Cancer 064003 | 0.0 |
| Liver Cancer 1025 | 0.0 |
| Liver Cancer 1026 | 4.2 |
| Liver Cancer 6004-T | 0.0 |
| Liver Tissue 6004-N | 2.9 |
| Liver Cancer 6005-T | 2.2 |
| Liver Tissue 6005-N | 0.0 |
| Normal Bladder | 23.8 |
| Bladder Cancer 1023 | 11.8 |
| Bladder Cancer A302173 | 7.2 |
| Bladder Cancer (OD04718-01) | 12.7 |
| Bladder Normal Adjacent (OD04718-03) | 4.9 |
| Normal Ovary | 3.5 |
| Ovarian Cancer 064008 | 100.0 |
| Ovarian Cancer (OD04768-07) | 2.5 |
| Ovary Margin (OD04768-08) | 8.1 |
| Normal Stomach | 1.5 |
| Gastric Cancer 9060358 | 0.5 |
| Stomach Margin 9060359 | 0.8 |
| Gastric Cancer 9060395 | 7.5 |
| Stomach Margin 9060394 | 1.9 |
| Gastric Cancer 9060397 | 27.0 |
| Stomach Margin 9060396 | 1.5 |
| Gastric Cancer 064005 | 3.3 |

Column A - Rel. Exp. (%) Ag3048, Run 170858352

TABLE ATD
Panel 3D

| Tissue Name | A |
|---|---|
| Daoy- Medulloblastoma | 6.5 |
| TE671- Medulloblastoma | 0.0 |
| D283 Med- Medulloblastoma | 0.0 |
| PFSK-1- Primitive Neuroectodermal | 18.0 |
| XF-498- CNS | 1.4 |
| SNB-78- Glioma | 100.0 |
| SF-268- Glioblastoma | 0.0 |
| T98G- Glioblastoma | 24.1 |
| SK-N-SH- Neuroblastoma (metastasis) | 32.3 |
| SF-295- Glioblastoma | 2.0 |
| Cerebellum | 3.0 |
| Cerebellum | 4.2 |
| NCI-H292- Mucoepidermoid lung | 38.4 |
| carcinoma | |
| DMS-114- Small cell lung cancer | 1.7 |
| DMS-79- Small cell lung cancer | 3.0 |
| NCI-H146- Small cell lung cancer | 3.3 |
| NCI-H526- Small cell lung cancer | 0.0 |
| NCI-N417- Small cell lung cancer | 0.0 |
| NCI-H82- Small cell lung cancer | 0.0 |
| NCI-H157- Squamous cell lung cancer | 1.4 |
| (metastasis) | |
| NCI-H1155- Large cell lung cancer | 1.8 |
| NCI-H1299- Large cell lung cancer | 2.8 |
| NCI-H727- Lung carcinoid | 0.0 |
| NCI-UMC-11- Lung carcinoid | 0.0 |
| LX-1- Small cell lung cancer | 0.0 |
| Colo-205- Colon cancer | 7.3 |
| KM12- Colon cancer | 1.6 |
| KM20L2- Colon cancer | 0.0 |
| NCI-H716- Colon cancer | 0.0 |
| SW-48- Colon adenocarcinoma | 0.6 |
| SW1116- Colon adenocarcinoma | 0.8 |
| LS 174T- Colon adenocarcinoma | 2.5 |
| SW-948- Colon adenocarcinoma | 0.0 |
| SW-480- Colon adenocarcinoma | 2.7 |
| NCI-SNU-5- Gastric carcinoma | 1.6 |
| KATO III- Gastric carcinoma | 1.8 |
| NCI-SNU-16- Gastric carcinoma | 1.4 |
| NCI-SNU-1- Gastric carcinoma | 0.0 |
| RF-1- Gastric adenocarcinoma | 0.0 |
| RF-48- Gastric adenocarcinoma | 0.9 |
| MKN-45- Gastric carcinoma | 5.6 |
| NCI-N87- Gastric carcinoma | 2.3 |
| OVCAR-5- Ovarian carcinoma | 4.8 |
| RL95-2- Uterine carcinoma | 0.0 |
| HelaS3- Cervical adenocarcinoma | 0.0 |
| Ca Ski- Cervical epidermoid carcinoma | 0.0 |
| (metastasis) | |
| ES-2- Ovarian clear cell carcinoma | 0.0 |
| Ramos- Stimulated with PMA/ionomycin 6 h | 0.0 |
| Ramos- Stimulated with PMA/ionomycin 14 h | 0.0 |
| MEG-01- Chronic myelogenous leukemia | 2.6 |
| (megokaryoblast) | |
| Raji- Burkitt's lymphoma | 0.0 |
| Daudi- Burkitt's lymphoma | 0.0 |
| U266- B-cell plasmacytoma | 0.0 |
| CA46- Burkitt's lymphoma | 0.0 |
| RL- non-Hodgkin's B-cell lymphoma | 0.0 |
| JM1- pre-B-cell lymphoma | 0.0 |
| Jurkat- T cell leukemia | 0.0 |
| TF-1- Erythroleukemia | 2.3 |
| HUT 78- T-cell lymphoma | 0.0 |
| U937- Histiocytic lymphoma | 0.7 |
| KU-812- Myelogenous leukemia | 0.0 |
| 769-P- Clear cell renal carcinoma | 0.0 |
| Caki-2- Clear cell renal carcinoma | 2.5 |
| SW 839- Clear cell renal carcinoma | 0.0 |
| Rhabdoid kidney tumor | 2.3 |
| Hs766T- Pancreatic carcinoma (LN | 1.5 |
| metastasis) | |
| CAPAN-1- Pancreatic adenocarcinoma (liver | 0.0 |
| metastasis) | |
| SU86.86- Pancreatic carcinoma (liver | 0.8 |
| metastasis) | |
| BxPC-3- Pancreatic adenocarcinoma | 0.7 |
| HPAC- Pancreatic adenocarcinoma | 0.0 |
| MIA PaCa-2- Pancreatic carcinoma | 0.7 |
| CFPAC-1- Pancreatic ductal adenocarcinoma | 1.7 |
| PANC-1- Pancreatic epithelioid ductal | 6.7 |
| carcinoma | |
| T24- Bladder carcinma (transitional cell) | 6.1 |
| 5637- Bladder carcinoma | 2.1 |
| HT-1197- Bladder carcinoma | 1.7 |
| UM-UC-3- Bladder carcinma (transitional cell) | 0.7 |
| A204- Rhabdomyosarcoma | 5.4 |
| HT-1080- Fibrosarcoma | 11.7 |
| MG-63- Osteosarcoma | 0.0 |
| SK-LMS-1- Leiomyosarcoma (vulva) | 7.5 |
| SJRH30- Rhabdomyosarcoma (met to bone | 0.0 |
| marrow) | |
| A431- Epidermoid carcinoma | 5.1 |
| WM266-4- Melanoma | 16.6 |
| DU 145- Prostate carcinoma (brain | 0.0 |
| metastasis) | |
| MDA-MB-468- Breast adenocarcinoma | 2.5 |
| SCC-4- Squamous cell carcinoma of tongue | 0.0 |
| SCC-9- Squamous cell carcinoma of tongue | 0.0 |
| SCC-15- Squamous cell carcinoma of tongue | 0.6 |
| CAL 27- Squamous cell carcinoma of tongue | 0.5 |

Column A - Rel. Exp. (%) Ag3048, Run 172133336

TABLE ATE
Panel 4D

| Tissue Name | A |
|---|---|
| Secondary Th1 act | 0.4 |
| Secondary Th2 act | 0.0 |
| Secondary Tr1 act | 0.6 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.9 |
| Primary Tr1 rest | 0.6 |
| CD45RA CD4 lymphocyte act | 8.6 |
| CD45RO CD4 lymphocyte act | 0.0 |
| CD8 lymphocyte act | 0.0 |
| Secondary CD8 lymphocyte rest | 0.6 |
| Secondary CD8 lymphocyte act | 0.6 |
| CD4 lymphocyte none | 0.5 |
| 2ry Th1/Th2/Tr1_anti-CD95 CH11 | 1.3 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.8 |
| PBMC rest | 0.8 |
| PBMC PWM | 0.0 |
| PBMC PHA-L | 2.2 |
| Ramos (B cell) none | 0.0 |
| Ramos (B cell) ionomycin | 0.8 |
| B lymphocytes PWM | 1.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 1.8 |
| Dendritic cells LPS | 1.7 |
| Dendritic cells anti-CD40 | 6.8 |
| Monocytes rest | 1.2 |
| Monocytes LPS | 0.0 |
| Macrophages rest | 1.9 |
| Macrophages LPS | 1.3 |
| HUVEC none | 0.0 |
| HUVEC starved | 2.9 |
| HUVEC IL-1beta | 0.0 |
| HUVEC IFN gamma | 1.9 |
| HUVEC TNF alpha + IFN gamma | 0.0 |
| HUVEC TNF alpha + IL4 | 1.2 |
| HUVEC IL-11 | 0.3 |
| Lung Microvascular EC none | 3.9 |
| Lung Microvascular EC TNFalpha + IL-1beta | 1.2 |
| Microvascular Dermal EC none | 0.6 |
| Microsvasular Dermal EC TNFalpha + IL-1beta | 1.4 |
| Bronchial epithelium TNFalpha + IL1beta | 28.7 |
| Small airway epithelium none | 39.2 |
| Small airway epithelium TNFalpha + IL-1beta | 84.7 |
| Coronery artery SMC rest | 17.9 |
| Coronery artery SMC TNFalpha + IL-1beta | 5.5 |
| Astrocytes rest | 6.1 |
| Astrocytes TNFalpha + IL-1beta | 5.5 |
| KU-812 (Basophil) rest | 0.7 |
| KU-812 (Basophil) PMA/ionomycin | 3.8 |
| CCD1106 (Keratinocytes) none | 79.0 |
| CCD1106 (Keratinocytes) TNFalpha + IL-1beta | 97.3 |
| Liver cirrhosis | 4.3 |
| Lupus kidney | 3.4 |
| NCI-H292 none | 87.1 |
| NCI-H292 IL-4 | 77.4 |
| NCI-H292 IL-9 | 82.9 |
| NCI-H292 IL-13 | 54.0 |
| NCI-H292 IFN gamma | 49.3 |
| HPAEC none | 0.7 |
| HPAEC TNF alpha + IL-1 beta | 0.0 |
| Lung fibroblast none | 35.1 |
| Lung fibroblast TNF alpha + IL-1 beta | 7.0 |
| Lung fibroblast IL-4 | 46.0 |
| Lung fibroblast IL-9 | 38.7 |
| Lung fibroblast IL-13 | 26.8 |
| Lung fibroblast IFN gamma | 68.8 |
| Dermal fibroblast CCD1070 rest | 100.0 |
| Dermal fibroblast CCD1070 TNF alpha | 59.9 |
| Dermal fibroblast CCD1070 IL-1 beta | 39.8 |
| Dermal fibroblast IFN gamma | 17.0 |
| Dermal fibroblast IL-4 | 30.6 |
| IBD Colitis 2 | 0.0 |
| IBD Crohn's | 0.7 |
| Colon | 2.0 |
| Lung | 36.1 |
| Thymus | 0.6 |
| Kidney | 1.7 |

Column A - Rel. Exp. (%) Ag3048, Run 164315038

Panel 1.3D Summary: Ag3048 The expression of this gene was highest in a sample derived from a brain cancer cell line (SF-539) (CT=29.4). There was substantial expression associated with samples derived from another brain cancer cell line, two melanoma cell lines and a lung cancer cell line. The expression of this gene is useful as a marker to distinguish SF-539 cells from other samples in the panel. Therapeutic modulation of this gene, through the use of small molecule drugs, protein therapeuitcs or antibodies is of benefit in treatment of brain or lung cancer or melanoma.

This gene, a leucine-rich repeat homolog, was expressed at low levels in the CNS. The leucine-rich repeat region proteins have been implicated in axonal guidance. This gene has therapeutic utility in the treatment of any CNS disorder involving neuronal loss, specfically by guiding/enhancing compensatory synaptogenesis and fiber outgrowth, including such clinical conditions as Alzheimer's, Parkinson's, or Huntington's diseases, stroke, head and spinal cord trauma, vascular dementia or spinocerebellar ataxia.

Panel 2D Summary: Ag3048 The expression of this gene was highest in a sample derived from an ovarian cancer (CT=29). There was substantial expression associated with lung cancer, prostate cancer and colon cancer samples. Of note is the differential expression in the lung, colon and prostate cancer samples compared to their respective normal adjacent tissue. The expression of this gene is useful as a marker to distinguish this ovarian cancer sample from other samples in the panel. The expression of this gene or its protein product is useful as a marker to distinguish colon, prostate or lung cancer samples from their normal adjacent tissue. Therapeutic modulation of this gene or its protein product, through the use of small molecule drugs, protein therapeutics or antibodies is benefical in the treatment of ovarian, lung, prostate or colon cancer.

Panel 3D Summary: Ag3048 The expression of this gene was highest in a sample derived from a brain cancer cell line (SNB-78) (CT=30.2). There was substantial expression associated with other brain cancer cell line samples and a lung cancer cell line sample. The expression of this gene is useful as a marker to distinguish SNB-78 cells from other samples in the panel. Therapeutic modulation of this gene or its protein product, through the use of small molecule drugs, protein therapeutics or antibodies is useftil in the treatment of brain or lung cancer.

Panel 4D Summary: Ag3048 This gene, a secreted leucine-rich repeat (LRR) protein, was expressed selectively at moderate levels (CT range 29-31) in several resting and cytokine-activated epithelial and connective tissue cells of lung and skin. This gene product is useful as a therapeutic protein as well as a target for therapeutic antibodies and small molecules, for the reduction or elimination of the symptoms in patients with chronic obstructive pulmonary disease, asthma, emphysema, or psoriasis.

AU. CG56914-01: Thrombospondin

Expression of gene CG56914-01 was assessed using the primer-probe sets Ag3108 and Ag3899, described in Tables AUA and AUB. Results of the RTQ-PCR runs are shown in Tables AUC, AUD, AUE, AUF, AUG.

TABLE AUA
Probe Name Ag3108

| | | | | SEQ |
|---|---|---|---|---|
| | | | Start | ID |
| Primers | Sequences | Length | Position | No |
| Forward | 5′-attccattgcccaaattaaca-3′ | 21 | 703 | 1513 |
| Probe | TET-5′-ccttcaataacaatattattccagccca-3′- | 28 | 728 | 1514 |
| | TAMRA | | | |
| Reverse | 5′-actgtgtccattcacactgtca-3′ | 22 | 759 | 1515 |

TABLE AUB
Probe Name Ag3899

| | | | | SEQ |
|---|---|---|---|---|
| | | | Start | ID |
| Primers | Sequences | Length | Position | No |
| Forward | 5′-ccattgcccaaattaacatg-3′ | 20 | 706 | 1516 |
| Probe | TET-5′-ccttcaataacaatattattccagccca-3′- | 28 | 728 | 1517 |
| | TAMRA | | | |
| Reverse | 5′-actgtgtccattcacactgtca-3′ | 22 | 759 | 1518 |

TABLE AUC
General_screening_panel_v_1.4

| Tissue Name | A |
|---|---|
| Adipose | 1.0 |
| Melanoma* Hs688(A).T | 33.9 |
| Melanoma* Hs688(B).T | 8.4 |
| Melanoma* M14 | 12.9 |
| Melanoma* LOXIMVI | 0.1 |
| Melanoma* SK-MEL-5 | 58.6 |
| Squamous cell carcinoma SCC-4 | 0.0 |
| Testis Pool | 0.6 |
| Prostate ca.* (bone met) PC-3 | 0.2 |
| Prostate Pool | 0.4 |
| Placenta | 0.1 |
| Uterus Pool | 0.1 |
| Ovarian ca. OVCAR-3 | 0.4 |
| Ovarian ca. SK-OV-3 | 0.1 |
| Ovarian ca. OVCAR-4 | 0.1 |
| Ovarian ca. OVCAR-5 | 0.2 |
| Ovarian ca. IGROV-1 | 0.1 |
| Ovarian ca. OVCAR-8 | 0.1 |
| Ovary | 3.6 |
| Breast ca. MCF-7 | 0.5 |
| Breast ca. MDA-MB-231 | 0.1 |
| Breast ca. BT 549 | 2.6 |
| Breast ca. T47D | 0.2 |
| Breast ca. MDA-N | 2.2 |
| Breast Pool | 0.1 |
| Trachea | 1.0 |
| Lung | 0.0 |
| Fetal Lung | 5.6 |
| Lung ca. NCI-N417 | 0.0 |
| Lung ca. LX-1 | 0.0 |
| Lung ca. NCI-H146 | 0.0 |
| Lung ca. SHP-77 | 0.0 |
| Lung ca. A549 | 0.0 |
| Lung ca. NCI-H526 | 0.0 |
| Lung ca. NCI-H23 | 0.3 |
| Lung ca. NCI-H460 | 0.1 |
| Lung ca. HOP-62 | 0.6 |
| Lung ca. NCI-H522 | 0.0 |
| Liver | 0.0 |
| Fetal Liver | 1.3 |
| Liver ca. HepG2 | 0.0 |
| Kidney Pool | 0.2 |
| Fetal Kidney | 1.4 |
| Renal ca. 786-0 | 0.2 |
| Renal ca. A498 | 0.0 |
| Renal ca. ACHN | 0.0 |
| Renal ca. UO-31 | 4.6 |
| Renal ca. TK-10 | 0.0 |
| Bladder | 0.6 |
| Gastric ca. (liver met.) NCI-N87 | 0.0 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 1.2 |
| Colon ca. SW1116 | 0.0 |
| Colon ca. Colo-205 | 0.0 |
| Colon ca. SW-48 | 0.0 |
| Colon Pool | 0.2 |
| Small Intestine Pool | 0.2 |
| Stomach Pool | 0.1 |
| Bone Marrow Pool | 0.2 |
| Fetal Heart | 1.0 |
| Heart Pool | 0.3 |
| Lymph Node Pool | 0.4 |
| Fetal Skeletal Muscle | 0.1 |
| Skeletal Muscle Pool | 0.2 |
| Spleen Pool | 1.1 |
| Thymus Pool | 0.6 |
| CNS cancer (glio/astro) U87-MG | 0.8 |
| CNS cancer (glio/astro) U-118-MG | 3.0 |
| CNS cancer (neuro; met) SK-N-AS | 0.0 |
| CNS cancer (astro) SF-539 | 18.8 |
| CNS cancer (astro) SNB-75 | 100.0 |
| CNS cancer (glio) SNB-19 | 0.0 |
| CNS cancer (glio) SF-295 | 0.8 |
| Brain (Amygdala) Pool | 0.0 |
| Brain (cerebellum) | 0.0 |
| Brain (fetal) | 0.0 |
| Brain (Hippocampus) Pool | 0.0 |
| Cerebral Cortex Pool | 0.0 |
| Brain (Substantia nigra) Pool | 0.0 |
| Brain (Thalamus) Pool | 0.0 |
| Brain (whole) | 0.0 |
| Spinal Cord Pool | 0.1 |
| Adrenal Gland | 0.1 |
| Pituitary gland Pool | 0.1 |
| Salivary Gland | 0.2 |
| Thyroid (female) | 0.1 |
| Pancreatic ca. CAPAN2 | 0.0 |
| Pancreas Pool | 0.4 |

Column A - Rel. Exp. (%) Ag3899, Run 219166475

TABLE AUD
HASS Panel v1.0

| Tissue Name | A |
|---|---|
| MCF-7 C1 | 43.8 |
| MCF-7 C2 | 51.4 |
| MCF-7 C3 | 11.2 |
| MCF-7 C4 | 72.2 |
| MCF-7 C5 | 11.0 |
| MCF-7 C6 | 43.8 |
| MCF-7 C7 | 18.8 |
| MCF-7 C9 | 17.3 |
| MCF-7 C10 | 100.0 |
| MCF-7 C11 | 3.2 |
| MCF-7 C12 | 22.1 |
| MCF-7 C13 | 26.4 |
| MCF-7 C15 | 10.4 |
| MCF-7 C16 | 45.1 |
| MCF-7 C17 | 22.2 |
| T24 D1 | 0.0 |
| T24 D2 | 0.0 |
| T24 D3 | 0.0 |
| T24 D4 | 0.0 |
| T24 D5 | 0.1 |
| T24 D6 | 0.0 |
| T24 D7 | 0.0 |
| T24 D9 | 0.0 |
| T24 D10 | 0.0 |
| T24 D11 | 0.0 |
| T24 D12 | 0.0 |
| T24 D13 | 0.0 |
| T24 D15 | 0.0 |
| T24 D16 | 0.0 |
| T24 D17 | 0.0 |
| CAPaN B1 | 0.0 |
| CAPaN B2 | 0.0 |
| CAPaN B3 | 0.0 |
| CAPaN B4 | 0.0 |
| CAPaN B5 | 0.0 |
| CAPaN B6 | 0.0 |
| CAPaN B7 | 0.0 |
| CAPaN B8 | 0.0 |
| CAPaN B9 | 0.0 |
| CAPaN B10 | 0.0 |
| CAPaN B11 | 0.0 |
| CAPaN B12 | 0.0 |
| CAPaN B13 | 0.0 |
| CAPaN B14 | 0.0 |
| CAPaN B15 | 0.0 |
| CAPaN B16 | 0.0 |
| CAPaN B17 | 0.0 |
| U87-MG F1 (B) | 0.2 |
| U87-MG F2 | 0.1 |
| U87-MG F3 | 0.8 |
| U87-MG F4 | 0.4 |
| U87-MG F5 | 3.5 |
| U87-MG F6 | 1.5 |
| U87-MG F7 | 0.6 |
| U87-MG F8 | 1.6 |
| U87-MG F9 | 0.0 |
| U87-MG F10 | 2.2 |
| U87-MG F11 | 3.1 |
| U87-MG F12 | 1.8 |
| U87-MG F13 | 0.7 |
| U87-MG F14 | 1.1 |
| U87-MG F15 | 0.7 |
| U87-MG F16 | 1.4 |
| U87-MG F17 | 1.8 |
| LnCAP A1 | 0.0 |
| LnCAP A2 | 0.0 |
| LnCAP A3 | 0.0 |
| LnCAP A4 | 0.0 |
| LnCAP A5 | 0.0 |
| LnCAP A6 | 0.0 |
| LnCAP A7 | 0.0 |
| LnCAP A8 | 0.0 |
| LnCAP A9 | 0.0 |
| LnCAP A10 | 0.0 |
| LnCAP A11 | 0.0 |
| LnCAP A12 | 0.0 |
| LnCAP A13 | 0.0 |
| LnCAP A14 | 0.0 |
| LnCAP A15 | 0.0 |
| LnCAP A16 | 0.0 |
| LnCAP A17 | 0.0 |
| Primary Astrocytes | 4.9 |
| Primary Renal Proximal Tubule Epithelial cell A2 | 0.3 |
| Primary melanocytes A5 | 0.4 |
| 126443 - 341 medullo | 0.0 |
| 126444 - 487 medullo | 0.0 |
| 126445 - 425 medullo | 2.7 |
| 126446 - 690 medullo | 0.5 |
| 126447 - 54 adult glioma | 0.2 |
| 126448 - 245 adult glioma | 38.4 |
| 126449 - 317 adult glioma | 0.0 |
| 126450 - 212 glioma | 0.0 |
| 126451 - 456 glioma | 0.3 |

Column A - Rel. Exp. (%) Ag3108, Run 268623842

TABLE AUE
Panel 2.1

| Tissue Name | A |
|---|---|
| Normal Colon | 0.7 |
| Colon cancer (OD06064) | 1.3 |
| Colon cancer margin (OD06064) | 0.0 |
| Colon cancer (OD06159) | 0.5 |
| Colon cancer margin (OD06159) | 1.8 |
| Colon cancer (OD06298-08) | 1.6 |
| Colon cancer margin (OD06298-018) | 0.3 |
| Colon Cancer Gr.2 ascend colon (ODO3921) | 1.6 |
| Colon Cancer margin (ODO3921) | 4.6 |
| Colon cancer metastasis (OD06104) | 2.1 |
| Lung margin (OD06104) | 2.8 |
| Colon mets to lung (OD04451-01) | 4.5 |
| Lung margin (OD04451-02) | 10.7 |
| Normal Prostate | 0.8 |
| Prostate Cancer (OD04410) | 0.7 |
| Prostate margin (OD04410) | 13.6 |
| Normal Lung | 34.2 |
| Invasive poor diff. lung adeno 1 (ODO4945-01) | 9.2 |
| Lung margin (ODO4945-03) | 6.2 |
| Lung Malignant Cancer (OD03126) | 11.1 |
| Lung margin (OD03126) | 34.9 |
| Lung Cancer (OD05014A) | 25.2 |
| Lung margin (OD05014B) | 5.6 |
| Lung Cancer (OD04237-01) | 1.5 |
| Lung margin (OD04237-02) | 63.3 |
| Ocular Mel Met to Liver (ODO4310) | 24.3 |
| Liver margin (ODO4310) | 7.6 |
| Melanoma Mets to Lung (OD04321) | 100.0 |
| Lung margin (OD04321) | 20.2 |
| Normal Kidney | 3.6 |
| Kidney Ca, Nuclear grade 2 (OD04338) | 6.9 |
| Kidney margin (OD04338) | 2.1 |
| Kidney Ca Nuclear grade 1/2 (OD04339) | 1.1 |
| Kidney margin (OD04339) | 0.2 |
| Kidney Ca, Clear cell type (OD04340) | 8.8 |
| Kidney margin (OD04340) | 4.5 |
| Kidney Ca, Nuclear grade 3 (OD04348) | 1.3 |
| Kidney margin (OD04348) | 1.8 |
| Kidney Cancer (OD04450-01) | 0.6 |
| Kidney margin (OD04450-03) | 4.6 |
| Kidney Cancer 8120613 | 0.3 |
| Kidney margin 8120614 | 0.5 |
| Kidney Cancer 9010320 | 0.9 |
| Kidney margin 9010321 | 9.5 |
| Kidney Cancer 8120607 | 0.6 |
| Kidney margin 8120608 | 0.7 |
| Normal Uterus | 1.7 |
| Uterus Cancer | 1.2 |
| Normal Thyroid | 0.1 |
| Thyroid Cancer | 0.9 |
| Thyroid Cancer A302152 | 1.2 |
| Thyroid margin A302153 | 0.9 |
| Normal Breast | 12.4 |
| Breast Cancer | 0.9 |
| Breast Cancer | 4.3 |
| Breast Cancer (OD04590-01) | 0.6 |
| Breast Cancer Mets (OD04590-03) | 6.6 |
| Breast Cancer Metastasis | 2.1 |
| Breast Cancer | 3.3 |
| Breast Cancer 9100266 | 4.6 |
| Breast margin 9100265 | 1.5 |
| Breast Cancer A209073 | 2.5 |
| Breast margin A2090734 | 9.9 |
| Normal Liver | 4.2 |
| Liver Cancer 1026 | 1.8 |
| Liver Cancer 1025 | 6.1 |
| Liver Cancer 6004-T | 3.5 |
| Liver Tissue 6004-N | 0.8 |
| Liver Cancer 6005-T | 14.2 |
| Liver Tissue 6005-N | 14.8 |
| Liver Cancer | 1.4 |
| Normal Bladder | 1.7 |
| Bladder Cancer | 1.8 |
| Bladder Cancer | 2.4 |
| Normal Ovary | 7.7 |
| Ovarian Cancer | 13.6 |
| Ovarian cancer (OD06145) | 0.6 |
| Ovarian cancer margin (OD06145) | 2.2 |
| Normal Stomach | 4.1 |
| Gastric Cancer 9060397 | 1.2 |
| Stomach margin 9060396 | 0.5 |
| Gastric Cancer 9060395 | 7.4 |
| Stomach margin 9060394 | 2.6 |
| Gastric Cancer 064005 | 4.3 |

Column A - Rel. Exp. (%) Ag3108, Run 170686074

TABLE AUF
Panel 4.1D

| Tissue Name | A |
|---|---|
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.3 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 3.2 |
| HUVEC starved | 8.1 |
| HUVEC IL-1beta | 4.1 |
| HUVEC IFN gamma | 15.8 |
| HUVEC TNF alpha + IFN gamma | 1.0 |
| HUVEC TNF alpha + IL4 | 2.9 |
| HUVEC IL-11 | 4.2 |
| Lung Microvascular EC none | 1.5 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.4 |
| Small airway epithelium none | 0.0 |
| Small airway epithelium TNFalpha + IL-1beta | 0.0 |
| Coronery artery SMC rest | 8.5 |
| Coronery artery SMC TNFalpha + IL-1beta | 1.8 |
| Astrocytes rest | 0.0 |
| Astrocytes TNFalpha + IL-1beta | 0.5 |
| KU-812 (Basophil) rest | 1.0 |
| KU-812 (Basophil) PMA/ionomycin | 8.0 |
| CCD1106 (Keratinocytes) none | 0.0 |
| CCD1106 (Keratinocytes) TNFalpha + IL-1beta | 0.0 |
| Liver cirrhosis | 7.6 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 17.9 |
| HPAEC TNF alpha + IL-1 beta | 11.3 |
| Lung fibroblast none | 3.4 |
| Lung fibroblast TNF alpha + IL-1 beta | 2.7 |
| Lung fibroblast IL-4 | 4.4 |
| Lung fibroblast IL-9 | 2.2 |
| Lung fibroblast IL-13 | 3.9 |
| Lung fibroblast IFN gamma | 7.2 |
| Dermal fibroblast CCD1070 rest | 5.5 |
| Dermal fibroblast CCD1070 TNF alpha | 1.9 |
| Dermal fibroblast CCD1070 IL-1 beta | 1.5 |
| Dermal fibroblast IFN gamma | 29.5 |
| Dermal fibroblast IL-4 | 75.8 |
| Dermal Fibroblasts rest | 21.5 |
| Neutrophils TNFa + LPS | 0.0 |
| Neutrophils rest | 0.0 |
| Colon | 2.0 |
| Lung | 100.0 |
| Thymus | 0.5 |
| Kidney | 3.4 |

Column A - Rel. Exp. (%) Ag3899, Run 170120166

TABLE AUG
general oncology screening panel_v_2.4

| Tissue Name | A | B |
|---|---|---|
| Colon cancer 1 | 13.9 | 10.8 |
| Colon NAT 1 | 7.3 | 4.5 |
| Colon cancer 2 | 2.3 | 5.2 |
| Colon NAT 2 | 1.1 | 0.8 |
| Colon cancer 3 | 14.1 | 11.7 |
| Colon NAT 3 | 16.6 | 4.2 |
| Colon malignant cancer 4 | 14.1 | 9.9 |
| Colon NAT 4 | 0.3 | 0.3 |
| Lung cancer 1 | 8.2 | 18.3 |
| Lung NAT 1 | 0.0 | 0.4 |
| Lung cancer 2 | 96.6 | 47.0 |
| Lung NAT 2 | 1.4 | 2.4 |
| Squamous cell carcinoma 3 | 50.0 | 22.2 |
| Lung NAT 3 | 0.6 | 0.7 |
| Metastatic melanoma 1 | 1.5 | 3.7 |
| Melanoma 2 | 0.0 | 0.2 |
| Melanoma 3 | 0.0 | 0.5 |
| Metastatic melanoma 4 | 100.0 | 100.0 |
| Metastatic melanoma 5 | 95.3 | 47.3 |
| Bladder cancer 1 | 0.0 | 0.0 |
| Bladder NAT 1 | 0.0 | 0.0 |
| Bladder cancer 2 | 0.0 | 0.2 |
| Bladder NAT 2 | 0.0 | 0.0 |
| Bladder NAT 3 | 0.0 | 0.0 |
| Bladder NAT 4 | 1.3 | 1.1 |
| Prostate adenocarcinoma 1 | 1.2 | 7.2 |
| Prostate adenocarcinoma 2 | 0.3 | 0.9 |
| Prostate adenocarcinoma 3 | 1.3 | 0.8 |
| Prostate adenocarcinoma 4 | 11.8 | 5.8 |
| Prostate NAT 5 | 5.7 | 1.7 |
| Prostate adenocarcinoma 6 | 0.3 | 0.8 |
| Prostate adenocarcinoma 7 | 1.0 | 1.6 |
| Prostate adenocarcinoma 8 | 0.4 | 0.7 |
| Prostate adenocarcinoma 9 | 23.3 | 41.5 |
| Prostate NAT 10 | 0.0 | 0.0 |
| Kidney cancer 1 | 48.3 | 7.0 |
| Kidney NAT 1 | 4.5 | 1.8 |
| Kidney cancer 2 | 40.6 | 29.5 |
| Kidney NAT 2 | 9.0 | 11.3 |
| Kidney cancer 3 | 25.3 | 11.5 |
| Kidney NAT 3 | 1.5 | 2.7 |
| Kidney cancer 4 | 40.1 | 11.2 |
| Kidney NAT 4 | 2.3 | 1.3 |

Column A - Rel. Exp. (%) Ag3108, Run 259737911
Column B - Rel. Exp. (%) Ag3899, Run 268143635

General_screening_panel_v1.4 Summary: Ag3899 Highest expression of this gene was detected in an astrocytoma SNB-75 cell line (CT=23.8). High levels of expression of this gene are also seen in a cluster of brain and melanoma cancer cell lines. Expression of this gene is useful as a marker to distinguish these samples from other samples in the panel and also as marker for detection of these cancers. Therapeutic modulation of the activity of this gene or its protein product, through the use of small molecule drugs, protein therapeutics or antibodies, is beneficial in the treatment of these cancers.

Among tissues with metabolic or endocrine function, this gene was expressed at low to moderate levels in pancreas, adipose, adrenal gland, thyroid, pituitary gland, skeletal muscle, heart, liver and the gastrointestinal tract. Therapeutic modulation of the activity of this gene or its gene product is useful in the treatment of endocrine/metabolically related diseases, such as obesity and diabetes.

This gene was expressed at much higher levels in fetal liver (CT=30) and lung (CTs=27.9) when compared to corresponding adult liver (CT=35) and lung (CT=36.8). Expression of this gene or ists protein product is of use as a marker to distinguish these fetal tissues from the corresponding adult tissues.

HASS Panel v1.0 Summary: Ag3108 This gene was expressed by MCF-7 cells and a glioma sample on this panel. Expression of this gene was serum-dependent in MCF-7 cells. Expression is regulated by cytokines and extracellular molecules found in serum. Modulation of this gene, through the use of small molecule drugs, protein therapeutics or antibodies is of benefit in the treatment of glioma.

Panel 2.1 Summary: Ag3108 Highest expression of this gene was detected in a melanoma metastasis sample (CT=29). Expression of this gene was higher in normal liver when compared to adjancent cancerous tissue and in metastasic breast cancer tissue (OD04590-03) (CT=33) as compared to primary breast cancer (OD04590-01) (CT=36.7). Expression of this gene is useful as a marker for cancer metastasis. Therapeutic modulation of the expression or function of this gene or gene product is useful in the treatment of lung, breast and melanoma cancers.

Panel 4.1D Summary: Ag3108 Highest expression of this gene was seen in lung (CT=28.6). Significant expression of this gene was also seen in HPAEC cells, HUVEC cells, lung fibroblast, TNFalpha+IL1 beta treated bronchial epithelium and dermal fibroblasts. Antibody, protein therapeutics or small molecule therapies designed with the protein encoded for by this gene are important in the treatment of inflammatory lung disorders such as chronic obstructive pulmonary disease, asthma, allergy and emphysema and skin disorders including psoriasis.

In addition, low expression of this gene was also seen in kidney and colon. Antibody, protein therapeutics or small molecule therapies designed with the protein encoded by this gene are important in the treatment of inflammatory or autoimmune diseases that affect the kidney, including lupus and glomerulonephritis, as well as, inflammatory bowel diseases such as Crohns.

Expression of this gene was stimulated in PMA/ionomycin treated basophils (CT=30) as compared to resting basophils (CT=36). Basophils release histamines and other biological modifiers in reponse to allergens and play an important role in the pathology of asthma and hypersensitivity reactions. Therapeutics designed against the protein encoded by this gene are useful for the reduction or inhibition of inflammation by blocking basophil function in these diseases. These cells are a reasonable model for the inflammatory cells that take part in various inflammatory lung and bowel diseases, such as asthma, Crohn's disease, and ulcerative colitis. Therapeutics that modulate the function of this gene or gene product are useful for the reduction or elimination of the symptoms of patients suffering from asthma, Crohn's disease, and ulcerative colitis.

general oncology screening panel_V_{—}2.4 Summary: Ag3108/Ag3960 Highest expression of this gene was seen in a metastatic melanoma sample (CTs=30-31). Expression of this gene was higher in kidney and lung cancer when compared to normal adjacent tissue samples. Expression of this gene is useful as a marker to differentiate these samples from other samples on this panel and as a marker for these cancers. Therapeutic modulation of the expression or function of this gene or gene product is of use in the treatment of these cancers.

AV. CG56959-02: Synaptotagmin X

Expression of full-length physical clone CG56959-02 was assessed using the primer-probe set Ag1557, described in Table AVA. Results of the RTQ-PCR runs are shown in Tables AVB, AVC, AVD and AVE.

TABLE AVA
Probe Name Ag1557

| | | | | SEQ |
|---|---|---|---|---|
| | | | Start | ID |
| Primers | Sequences | Length | Position | No |
| Forward | 5′-gcgtgcacagaaagactttaaa-3′ | 22 | 698 | 1519 |
| Probe | TET-5′-tgatgaaacttttcaatttcctgtagca-3′- | 28 | 661 | 1520 |
| | TAMRA | | | |
| Reverse | 5′-tgaaatgtagttttcggttgct-3′ | 22 | 627 | 1521 |

TABLE AVB
AI_comprehensive panel_v1.0

| Tissue Name | A |
|---|---|
| 110967 COPD-F | 0.0 |
| 110980 COPD-F | 0.0 |
| 110968 COPD-M | 0.0 |
| 110977 COPD-M | 0.0 |
| 110989 Emphysema-F | 0.0 |
| 110992 Emphysema-F | 0.0 |
| 110993 Emphysema-F | 0.0 |
| 110994 Emphysema-F | 0.0 |
| 110995 Emphysema-F | 0.8 |
| 110996 Emphysema-F | 0.0 |
| 110997 Asthma-M | 2.6 |
| 111001 Asthma-F | 0.0 |
| 111002 Asthma-F | 0.0 |
| 111003 Atopic Asthma-F | 0.0 |
| 111004 Atopic Asthma-F | 0.0 |
| 111005 Atopic Asthma-F | 0.0 |
| 111006 Atopic Asthma-F | 0.0 |
| 111417 Allergy-M | 0.0 |
| 112347 Allergy-M | 2.4 |
| 112349 Normal Lung-F | 2.4 |
| 112357 Normal Lung-F | 2.1 |
| 112354 Normal Lung-M | 0.0 |
| 112374 Crohns-F | 23.8 |
| 112389 Match Control Crohns-F | 7.0 |
| 112375 Crohns-F | 32.1 |
| 112732 Match Control Crohns-F | 0.0 |
| 112725 Crohns-M | 11.7 |
| 112387 Match Control Crohns-M | 0.0 |
| 112378 Crohns-M | 5.0 |
| 112390 Match Control Crohns-M | 1.3 |
| 112726 Crohns-M | 26.1 |
| 112731 Match Control Crohns-M | 67.8 |
| 112380 Ulcer Col-F | 0.0 |
| 112734 Match Control Ulcer Col-F | 0.0 |
| 112384 Ulcer Col-F | 1.1 |
| 112737 Match Control Ulcer Col-F | 12.9 |
| 112386 Ulcer Col-F | 0.0 |
| 112738 Match Control Ulcer Col-F | 0.9 |
| 112381 Ulcer Col-M | 9.4 |
| 112735 Match Control Ulcer Col-M | 62.4 |
| 112382 Ulcer Col-M | 4.5 |
| 112394 Match Control Ulcer Col-M | 0.0 |
| 112383 Ulcer Col-M | 0.7 |
| 112736 Match Control Ulcer Col-M | 2.1 |
| 112423 Psoriasis-F | 1.6 |
| 112427 Match Control Psoriasis-F | 2.7 |
| 112418 Psoriasis-M | 0.0 |
| 112723 Match Control Psoriasis-M | 4.4 |
| 112419 Psoriasis-M | 5.8 |
| 112424 Match Control Psoriasis-M | 2.3 |
| 112420 Psoriasis-M | 1.3 |
| 112425 Match Control Psoriasis-M | 0.0 |
| 104689 (MF) OA Bone-Backus | 0.0 |
| 104690 (MF) Adj “Normal” Bone-Backus | 0.8 |
| 104691 (MF) OA Synovium-Backus | 0.0 |
| 104692 (BA) OA Cartilage-Backus | 0.0 |
| 104694 (BA) OA Bone-Backus | 0.8 |
| 104695 (BA) Adj “Normal” Bone-Backus | 0.0 |
| 104696 (BA) OA Synovium-Backus | 1.1 |
| 104700 (SS) OA Bone-Backus | 0.0 |
| 104701 (SS) Adj “Normal” Bone-Backus | 0.0 |
| 104702 (SS) OA Synovium-Backus | 0.9 |
| 117093 OA Cartilage Rep7 | 0.0 |
| 112672 OA Bone5 | 9.4 |
| 112673 OA Synovium5 | 0.9 |
| 112674 OA Synovial Fluid cells5 | 2.8 |
| 117100 OA Cartilage Rep14 | 0.0 |
| 112756 OA Bone9 | 100.0 |
| 112757 OA Synovium9 | 0.0 |
| 112758 OA Synovial Fluid Cells9 | 0.0 |
| 117125 RA Cartilage Rep2 | 0.0 |
| 113492 Bone2 RA | 0.0 |
| 113493 Synovium2 RA | 0.0 |
| 113494 Syn Fluid Cells RA | 0.6 |
| 113499 Cartilage4 RA | 0.0 |
| 113500 Bone4 RA | 0.0 |
| 113501 Synovium4 RA | 1.6 |
| 113502 Syn Fluid Cells4 RA | 0.0 |
| 113495 Cartilage3 RA | 1.6 |
| 113496 Bone3 RA | 0.0 |
| 113497 Synovium3 RA | 2.1 |
| 113498 Syn Fluid Cells3 RA | 0.0 |
| 117106 Normal Cartilage Rep20 | 0.0 |
| 113663 Bone3 Normal | 6.5 |
| 113664 Synovium3 Normal | 1.4 |
| 113665 Syn Fluid Cells3 Normal | 5.8 |
| 117107 Normal Cartilage Rep22 | 0.0 |
| 113667 Bone4 Normal | 0.0 |
| 113668 Synovium4 Normal | 0.0 |
| 113669 Syn Fluid Cells4 Normal | 0.0 |

Column A - Rel. Exp. (%) Ag1557, Run 257809393

TABLE AVC
Panel 1.3D

| Tissue Name | A | B |
|---|---|---|
| Liver adenocarcinoma | 0.0 | 0.0 |
| Pancreas | 0.0 | 4.9 |
| Pancreatic ca. CAPAN 2 | 0.0 | 0.0 |
| Adrenal gland | 2.0 | 0.0 |
| Thyroid | 0.0 | 0.0 |
| Salivary gland | 0.0 | 0.0 |
| Pituitary gland | 68.3 | 60.7 |
| Brain (fetal) | 0.0 | 0.0 |
| Brain (whole) | 43.8 | 34.6 |
| Brain (amygdala) | 8.9 | 13.8 |
| Brain (cerebellum) | 0.0 | 0.0 |
| Brain (hippocampus) | 35.6 | 70.7 |
| Brain (substantia nigra) | 2.1 | 0.0 |
| Brain (thalamus) | 6.8 | 18.9 |
| Cerebral Cortex | 98.6 | 100.0 |
| Spinal cord | 21.5 | 13.2 |
| glio/astro U87-MG | 0.0 | 0.0 |
| glio/astro U-118-MG | 0.0 | 0.0 |
| astrocytoma SW1783 | 0.0 | 0.0 |
| neuro*; met SK-N-AS | 0.0 | 0.0 |
| astrocytoma SF-539 | 0.0 | 0.0 |
| astrocytoma SNB-75 | 10.3 | 12.4 |
| glioma SNB-19 | 0.0 | 0.0 |
| glioma U251 | 1.6 | 0.0 |
| glioma SF-295 | 0.0 | 0.0 |
| Heart (fetal) | 1.6 | 0.0 |
| Heart | 0.0 | 0.0 |
| Skeletal muscle (fetal) | 0.0 | 0.0 |
| Skeletal muscle | 0.0 | 0.0 |
| Bone marrow | 0.0 | 0.0 |
| Thymus | 0.0 | 0.0 |
| Spleen | 0.0 | 0.0 |
| Lymph node | 0.0 | 0.0 |
| Colorectal | 5.6 | 9.7 |
| Stomach | 15.5 | 12.9 |
| Small intestine | 22.2 | 50.3 |
| Colon ca. SW480 | 0.0 | 0.0 |
| Colon ca.* SW620(SW480 met) | 0.0 | 0.0 |
| Colon ca. HT29 | 0.0 | 0.0 |
| Colon ca. HCT-116 | 0.0 | 0.0 |
| Colon ca. CaCo-2 | 3.7 | 2.4 |
| Colon ca. tissue(ODO3866) | 0.0 | 0.0 |
| Colon ca. HCC-2998 | 0.0 | 0.0 |
| Gastric ca.* (liver met) NCI-N87 | 0.0 | 0.0 |
| Bladder | 0.0 | 7.8 |
| Trachea | 2.6 | 3.8 |
| Kidney | 2.4 | 18.4 |
| Kidney (fetal) | 0.0 | 7.7 |
| Renal ca. 786-0 | 2.3 | 0.0 |
| Renal ca. A498 | 0.0 | 0.0 |
| Renal ca. RXF 393 | 0.0 | 0.0 |
| Renal ca. ACHN | 0.0 | 0.0 |
| Renal ca. UO-31 | 0.0 | 0.0 |
| Renal ca. TK-10 | 0.0 | 0.0 |
| Liver | 1.8 | 8.0 |
| Liver (fetal) | 0.0 | 0.0 |
| Liver ca. (hepatoblast) HepG2 | 0.0 | 0.0 |
| Lung | 0.0 | 0.0 |
| Lung (fetal) | 0.0 | 0.0 |
| Lung ca. (small cell) LX-1 | 0.0 | 0.0 |
| Lung ca. (small cell) NCI-H69 | 0.0 | 0.0 |
| Lung ca. (s. cell var.) SHP-77 | 0.0 | 0.0 |
| Lung ca. (large cell)NCI-H460 | 0.0 | 0.0 |
| Lung ca. (non-sm. cell) A549 | 0.0 | 0.0 |
| Lung ca. (non-s. cell) NCI-H23 | 0.0 | 0.0 |
| Lung ca. (non-s. cell) HOP-62 | 0.0 | 0.0 |
| Lung ca. (non-s. cl) NCI-H522 | 1.9 | 5.9 |
| Lung ca. (squam.) SW 900 | 5.3 | 0.0 |
| Lung ca. (squam.) NCI-H596 | 0.0 | 0.0 |
| Mammary gland | 0.0 | 0.0 |
| Breast ca.* (pl. ef) MCF-7 | 100.0 | 62.0 |
| Breast ca.* (pl. ef) MDA-MB-231 | 0.0 | 0.0 |
| Breast ca.* (pl. ef) T47D | 0.0 | 0.0 |
| Breast ca. BT-549 | 3.5 | 0.0 |
| Breast ca. MDA-N | 0.0 | 0.0 |
| Ovary | 0.0 | 9.7 |
| Ovarian ca. OVCAR-3 | 41.5 | 23.7 |
| Ovarian ca. OVCAR-4 | 0.0 | 0.0 |
| Ovarian ca. OVCAR-5 | 0.0 | 0.0 |
| Ovarian ca. OVCAR-8 | 0.0 | 0.0 |
| Ovarian ca. IGROV-1 | 0.0 | 0.0 |
| Ovarian ca.* (ascites) SK-OV-3 | 0.0 | 0.0 |
| Uterus | 4.2 | 4.6 |
| Placenta | 0.0 | 0.0 |
| Prostate | 7.2 | 11.2 |
| Prostate ca.* (bone met)PC-3 | 0.0 | 0.0 |
| Testis | 13.3 | 11.1 |
| Melanoma Hs688(A).T | 0.0 | 0.0 |
| Melanoma* (met) Hs688(B).T | 0.0 | 0.0 |
| Melanoma UACC-62 | 0.0 | 0.0 |
| Melanoma M14 | 0.0 | 0.0 |
| Melanoma LOX IMVI | 0.0 | 0.0 |
| Melanoma* (met) SK-MEL-5 | 0.0 | 0.0 |
| Adipose | 0.0 | 0.0 |

Column A - Rel. Exp. (%) Ag1557, Run 146380320
Column B - Rel. Exp. (%) Ag1557, Run 149871705

TABLE AVD
Panel 2D

| Tissue Name | A | B |
|---|---|---|
| Normal Colon | 22.5 | 51.4 |
| CC Well to Mod Diff (ODO3866) | 0.0 | 2.1 |
| CC Margin (ODO3866) | 9.0 | 17.6 |
| CC Gr.2 rectosigmoid (ODO3868) | 0.0 | 0.0 |
| CC Margin (ODO3868) | 3.7 | 10.3 |
| CC Mod Diff (ODO3920) | 0.0 | 0.0 |
| CC Margin (ODO3920) | 6.8 | 15.6 |
| CC Gr.2 ascend colon (ODO3921) | 9.6 | 11.8 |
| CC Margin (ODO3921) | 4.6 | 6.3 |
| CC from Partial Hepatectomy | 0.0 | 1.2 |
| (ODO4309) Mets | | |
| Liver Margin (ODO4309) | 2.3 | 1.2 |
| Colon mets to lung (OD04451-01) | 0.0 | 2.1 |
| Lung Margin (OD04451-02) | 0.0 | 0.0 |
| Normal Prostate 6546-1 | 8.2 | 8.2 |
| Prostate Cancer (OD04410) | 2.3 | 5.8 |
| Prostate Margin (OD04410) | 13.8 | 16.6 |
| Prostate Cancer (OD04720-01) | 7.2 | 14.9 |
| Prostate Margin (OD04720-02) | 54.0 | 60.3 |
| Normal Lung 061010 | 4.3 | 0.0 |
| Lung Met to Muscle (ODO4286) | 0.0 | 2.6 |
| Muscle Margin (ODO4286) | 0.0 | 0.0 |
| Lung Malignant Cancer (OD03126) | 0.0 | 0.0 |
| Lung Margin (OD03126) | 2.0 | 2.4 |
| Lung Cancer (OD04404) | 0.0 | 0.0 |
| Lung Margin (OD04404) | 0.0 | 0.0 |
| Lung Cancer (OD04565) | 0.0 | 0.0 |
| Lung Margin (OD04565) | 0.0 | 0.0 |
| Lung Cancer (OD04237-01) | 51.1 | 51.1 |
| Lung Margin (OD04237-02) | 0.0 | 1.4 |
| Ocular Mel Met to Liver (ODO4310) | 0.0 | 0.0 |
| Liver Margin (ODO4310) | 3.5 | 6.3 |
| Melanoma Mets to Lung (OD04321) | 0.0 | 0.0 |
| Lung Margin (OD04321) | 0.0 | 2.7 |
| Normal Kidney | 87.7 | 76.8 |
| Kidney Ca, Nuclear grade 2 (OD04338) | 4.7 | 8.8 |
| Kidney Margin (OD04338) | 18.9 | 19.2 |
| Kidney Ca Nuclear grade 1/2 (OD04339) | 14.7 | 14.6 |
| Kidney Margin (OD04339) | 14.5 | 13.3 |
| Kidney Ca, Clear cell type (OD04340) | 0.0 | 0.0 |
| Kidney Margin (OD04340) | 21.0 | 15.0 |
| Kidney Ca, Nuclear grade 3 (OD04348) | 0.0 | 0.0 |
| Kidney Margin (OD04348) | 13.1 | 22.4 |
| Kidney Cancer (OD04622-01) | 0.0 | 0.0 |
| Kidney Margin (OD04622-03) | 3.0 | 9.4 |
| Kidney Cancer (OD04450-01) | 0.0 | 0.0 |
| Kidney Margin (OD04450-03) | 20.4 | 31.4 |
| Kidney Cancer 8120607 | 3.7 | 7.7 |
| Kidney Margin 8120608 | 1.8 | 3.0 |
| Kidney Cancer 8120613 | 100.0 | 100.0 |
| Kidney Margin 8120614 | 2.0 | 1.4 |
| Kidney Cancer 9010320 | 3.0 | 5.0 |
| Kidney Margin 9010321 | 2.3 | 1.5 |
| Normal Uterus | 0.0 | 0.0 |
| Uterus Cancer 064011 | 0.0 | 0.0 |
| Normal Thyroid | 0.0 | 0.0 |
| Thyroid Cancer 064010 | 1.0 | 0.0 |
| Thyroid Cancer A302152 | 0.0 | 0.0 |
| Thyroid Margin A302153 | 0.0 | 0.0 |
| Normal Breast | 2.1 | 0.0 |
| Breast Cancer (OD04566) | 0.0 | 0.0 |
| Breast Cancer (OD04590-01) | 0.0 | 0.0 |
| Breast Cancer Mets (OD04590-03) | 1.4 | 0.0 |
| Breast Cancer Metastasis (OD04655-05) | 1.9 | 2.1 |
| Breast Cancer 064006 | 41.2 | 0.0 |
| Breast Cancer 1024 | 0.0 | 0.0 |
| Breast Cancer 9100266 | 1.3 | 0.8 |
| Breast Margin 9100265 | 0.0 | 0.0 |
| Breast Cancer A209073 | 8.0 | 8.3 |
| Breast Margin A209073 | 1.6 | 1.3 |
| Normal Liver | 8.5 | 15.0 |
| Liver Cancer 064003 | 1.5 | 2.3 |
| Liver Cancer 1025 | 3.4 | 6.4 |
| Liver Cancer 1026 | 0.0 | 1.7 |
| Liver Cancer 6004-T | 7.1 | 2.0 |
| Liver Tissue 6004-N | 0.0 | 0.0 |
| Liver Cancer 6005-T | 0.0 | 2.0 |
| Liver Tissue 6005-N | 1.5 | 4.9 |
| Normal Bladder | 1.9 | 4.5 |
| Bladder Cancer 1023 | 14.0 | 19.6 |
| Bladder Cancer A302173 | 2.9 | 9.4 |
| Bladder Cancer (OD04718-01) | 50.7 | 74.2 |
| Bladder Normal Adjacent (OD04718-03) | 0.0 | 0.0 |
| Normal Ovary | 1.5 | 2.6 |
| Ovarian Cancer 064008 | 1.2 | 0.0 |
| Ovarian Cancer (OD04768-07) | 0.0 | 0.0 |
| Ovary Margin (OD04768-08) | 0.0 | 0.0 |
| Normal Stomach | 13.8 | 15.3 |
| Gastric Cancer 9060358 | 1.6 | 0.0 |
| Stomach Margin 9060359 | 6.7 | 8.4 |
| Gastric Cancer 9060395 | 1.4 | 1.2 |
| Stomach Margin 9060394 | 2.5 | 9.0 |
| Gastric Cancer 9060397 | 0.0 | 0.0 |
| Stomach Margin 9060396 | 0.0 | 0.0 |
| Gastric Cancer 064005 | 2.1 | 8.0 |

Column A - Rel. Exp. (%) Ag1557, Run 148394031
Column B - Rel. Exp. (%) Ag1557, Run 149871817

TABLE AVE
Panel 4D

| Tissue Name | A |
|---|---|
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 1.8 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 3.6 |
| Lung Microvascular EC TNFalpha + IL-1beta | 0.0 |
| Microvascular Dermal EC none | 2.7 |
| Microsvasular Dermal EC TNFalpha + IL-1beta | 0.0 |
| Bronchial epithelium TNFalpha + IL1beta | 0.0 |
| Small airway epithelium none | 3.6 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL-1beta | 0.0 |
| Liver cirrhosis | 5.2 |
| Lupus kidney | 1.9 |
| NCI-H292 none | 1.6 |
| NCI-H292 IL-4 | 1.6 |
| NCI-H292 IL-9 | 0.0 |
| NCI-H292 IL-13 | 0.0 |
| NCI-H292 IFN gamma | 0.0 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| IBD Colitis 2 | 7.3 |
| IBD Crohn's | 4.0 |
| Colon | 28.1 |
| Lung | 2.0 |
| Thymus | 100.0 |
| Kidney | 0.0 |

Column A - Rel. Exp. (%) Ag1557, Run 149871968

AI_comprehensive panel_v1.0 Summary: Ag1557 Low but significant expression was seen in an OA bone sample (CT=32). Prominent expression was also detected in ulcerative colitis and Crohn's samples as well as adjacent normal tissue samples.

Panel 1.3D Summary: Ag1557 Highest expression of this gene was detected in a breast cancer cell line and the cerebral cortex (CTs=33-34.6). Expression of this gene is of use as a marker for breast cancer. This gene was also expressed at moderate levels in the pituitary gland. The protein encoded by this gene is homologous to synaptotagmin and is important in the pituitary secretory pathway. This gene product or antibodies or small molecules that target this gene product are useful in the treatment of disease in this tissue.

Panel 2D Summary: Ag1557 Increased expression of this gene was seen in a single kidney sample (CTs=33), lung cancer sample and bladder cancer sample compared to the expression in the normal adjacent tissues showing that expression of this gene or gene product is of use as a marker to distinguish cancer from normal adjacent tissue. Therapeutic inhibition of this gene activity, through the use of small molecule drugs, protein therapeutics or antibodies, is of utility in the treatment of lung, bladder and kidney cancers.

Panel 4D Summary: Ag1557 This gene was expressed at a moderate level (CT=33.39) in thymus (Panel 4D). Small molecule drugs, protein therapeutics, or antibodies that inhibit the function of this synaptotagmin-like protein are useful for the reduction or elimination of the symptoms of autoimmune or inflammatory diseases that depend on the T cells that develop in the thymus, such as asthma, allergies, inflammatory bowel disease, lupus erythematosus, or rheumatoid arthritis.

AW. CG57111-01: Protocadherin

Expression of gene CG571 11-01 was assessed using the primer-probe sets Ag1096 and Ag3242, described in Tables AWA and AWB. Results of the RTQ-PCR runs are shown in Tables AWC, AWD, AWE, and AWF.

TABLE AWA
Probe Name Ag1096

| | | | | SEQ |
|---|---|---|---|---|
| | | | Start | ID |
| Primers | Sequences | Length | Position | No |
| Forward | 5′-actttggaagaggcattgct-3′ | 20 | 2403 | 1522 |
| Probe | TET-5′-cagacagattatgggctccatcgctt-3′- | 26 | 2424 | 1523 |
| | TAMRA | | | |
| Reverse | 5′-ctcgggataaccatgatcact-3′ | 21 | 2463 | 1524 |

TABLE AWB
Probe Name Ag3242

| | | | | SEQ |
|---|---|---|---|---|
| | | | Start | ID |
| Primers | Sequences | Length | Position | No |
| Forward | 5′-gggaccaatgctcaaattactt-3′ | 22 | 1038 | 1525 |
| Probe | TET-5′-tcagaaagttccacaagcatctaagga-3′- | 27 | 1070 | 1526 |
| | TAMRA | | | |
| Reverse | 5′-atgactccagtgttttcatcca-3′ | 22 | 1108 | 1527 |

TABLE AWC
AI_comprehensive panel_v1.0

| Tissue Name | A |
|---|---|
| 110967 COPD-F | 28.9 |
| 110980 COPD-F | 6.7 |
| 110968 COPD-M | 19.9 |
| 110977 COPD-M | 8.4 |
| 110989 Emphysema-F | 49.3 |
| 110992 Emphysema-F | 22.8 |
| 110993 Emphysema-F | 31.0 |
| 110994 Emphysema-F | 12.7 |
| 110995 Emphysema-F | 25.5 |
| 110996 Emphysema-F | 15.8 |
| 110997 Asthma-M | 7.5 |
| 111001 Asthma-F | 30.4 |
| 111002 Asthma-F | 45.4 |
| 111003 Atopic Asthma-F | 45.1 |
| 111004 Atopic Asthma-F | 22.4 |
| 111005 Atopic Asthma-F | 25.3 |
| 111006 Atopic Asthma-F | 13.9 |
| 111417 Allergy-M | 15.1 |
| 112347 Allergy-M | 5.0 |
| 112349 Normal Lung-F | 1.9 |
| 112357 Normal Lung-F | 5.1 |
| 112354 Normal Lung-M | 11.6 |
| 112374 Crohns-F | 32.5 |
| 112389 Match Control Crohns-F | 14.4 |
| 112375 Crohns-F | 36.1 |
| 112732 Match Control Crohns-F | 0.0 |
| 112725 Crohns-M | 45.4 |
| 112387 Match Control Crohns-M | 18.3 |
| 112378 Crohns-M | 3.8 |
| 112390 Match Control Crohns-M | 22.4 |
| 112726 Crohns-M | 45.7 |
| 112731 Match Control Crohns-M | 12.6 |
| 112380 Ulcer Col-F | 28.3 |
| 112734 Match Control Ulcer Col-F | 0.8 |
| 112384 Ulcer Col-F | 100.0 |
| 112737 Match Control Ulcer Col-F | 28.1 |
| 112386 Ulcer Col-F | 25.5 |
| 112738 Match Control Ulcer Col-F | 2.1 |
| 112381 Ulcer Col-M | 2.3 |
| 112735 Match Control Ulcer Col-M | 53.6 |
| 112382 Ulcer Col-M | 15.3 |
| 112394 Match Control Ulcer Col-M | 10.2 |
| 112383 Ulcer Col-M | 45.4 |
| 112736 Match Control Ulcer Col-M | 4.0 |
| 112423 Psoriasis-F | 90.1 |
| 112427 Match Control Psoriasis-F | 55.9 |
| 112418 Psoriasis-M | 25.7 |
| 112723 Match Control Psoriasis-M | 0.0 |
| 112419 Psoriasis-M | 66.0 |
| 112424 Match Control Psoriasis-M | 42.0 |
| 112420 Psoriasis-M | 44.4 |
| 112425 Match Control Psoriasis-M | 24.3 |
| 104689 (MF) OA Bone-Backus | 81.2 |
| 104690 (MF) Adj “Normal” Bone-Backus | 16.6 |
| 104691 (MF) OA Synovium-Backus | 1.5 |
| 104692 (BA) OA Cartilage-Backus | 7.4 |
| 104694 (BA) OA Bone-Backus | 56.3 |
| 104695 (BA) Adj “Normal” Bone-Backus | 31.6 |
| 104696 (BA) OA Synovium-Backus | 0.0 |
| 104700 (SS) OA Bone-Backus | 63.7 |
| 104701 (SS) Adj “Normal” Bone-Backus | 63.3 |
| 104702 (SS) OA Synovium-Backus | 6.1 |
| 117093 OA Cartilage Rep7 | 16.3 |
| 112672 OA Bone5 | 13.2 |
| 112673 OA Synovium5 | 5.4 |
| 112674 OA Synovial Fluid cells5 | 7.7 |
| 117100 OA Cartilage Rep14 | 4.5 |
| 112756 OA Bone9 | 17.1 |
| 112757 OA Synovium9 | 21.8 |
| 112758 OA Synovial Fluid Cells9 | 13.3 |
| 117125 RA Cartilage Rep2 | 28.1 |
| 113492 Bone2 RA | 34.2 |
| 113493 Synovium2 RA | 8.9 |
| 113494 Syn Fluid Cells RA | 25.7 |
| 113499 Cartilage4 RA | 45.1 |
| 113500 Bone4 RA | 47.3 |
| 113501 Synovium4 RA | 42.6 |
| 113502 Syn Fluid Cells4 RA | 25.5 |
| 113495 Cartilage3 RA | 33.7 |
| 113496 Bone3 RA | 38.7 |
| 113497 Synovium3 RA | 18.2 |
| 113498 Syn Fluid Cells3 RA | 30.8 |
| 117106 Normal Cartilage Rep20 | 0.0 |
| 113663 Bone3 Normal | 2.8 |
| 113664 Synovium3 Normal | 0.0 |
| 113665 Syn Fluid Cells3 Normal | 2.9 |
| 117107 Normal Cartilage Rep22 | 10.5 |
| 113667 Bone4 Normal | 17.2 |
| 113668 Synovium4 Normal | 17.0 |
| 113669 Syn Fluid Cells4 Normal | 36.3 |

Column A - Rel. Exp. (%) Ag3242, Run 253058919

TABLE AWD
Panel 1.3D

| Tissue Name | A |
|---|---|
| Liver adenocarcinoma | 0.0 |
| Pancreas | 1.0 |
| Pancreatic ca. CAPAN 2 | 0.0 |
| Adrenal gland | 0.3 |
| Thyroid | 0.0 |
| Salivary gland | 0.6 |
| Pituitary gland | 4.8 |
| Brain (fetal) | 11.8 |
| Brain (whole) | 40.6 |
| Brain (amygdala) | 31.6 |
| Brain (cerebellum) | 3.0 |
| Brain (hippocampus) | 76.3 |
| Brain (substantia nigra) | 2.1 |
| Brain (thalamus) | 6.3 |
| Cerebral Cortex | 27.7 |
| Spinal cord | 7.7 |
| glio/astro U87-MG | 0.6 |
| glio/astro U-118-MG | 0.5 |
| astrocytoma SW1783 | 0.0 |
| neuro*; met SK-N-AS | 7.5 |
| astrocytoma SF-539 | 0.0 |
| astrocytoma SNB-75 | 8.4 |
| glioma SNB-19 | 3.0 |
| glioma U251 | 66.4 |
| glioma SF-295 | 0.6 |
| Heart (fetal) | 3.7 |
| Heart | 1.7 |
| Skeletal muscle (fetal) | 6.9 |
| Skeletal muscle | 1.9 |
| Bone marrow | 0.0 |
| Thymus | 0.4 |
| Spleen | 0.0 |
| Lymph node | 0.0 |
| Colorectal | 3.5 |
| Stomach | 2.5 |
| Small intestine | 7.2 |
| Colon ca. SW480 | 0.0 |
| Colon ca.* SW620(SW480 met) | 0.0 |
| Colon ca. HT29 | 0.0 |
| Colon ca. HCT-116 | 0.0 |
| Colon ca. CaCo-2 | 0.0 |
| Colon ca. tissue(ODO3866) | 0.0 |
| Colon ca. HCC-2998 | 2.1 |
| Gastric ca.* (liver met) NCI-N87 | 0.0 |
| Bladder | 0.0 |
| Trachea | 9.0 |
| Kidney | 1.5 |
| Kidney (fetal) | 2.7 |
| Renal ca. 786-0 | 0.0 |
| Renal ca. A498 | 0.0 |
| Renal ca. RXF 393 | 0.4 |
| Renal ca. ACHN | 100.0 |
| Renal ca. UO-31 | 0.0 |
| Renal ca. TK-10 | 1.1 |
| Liver | 1.0 |
| Liver (fetal) | 0.0 |
| Liver ca. (hepatoblast) HepG2 | 0.0 |
| Lung | 4.8 |
| Lung (fetal) | 2.2 |
| Lung ca. (small cell) LX-1 | 3.7 |
| Lung ca. (small cell) NCI-H69 | 0.0 |
| Lung ca. (s. cell var.) SHP-77 | 16.0 |
| Lung ca. (large cell)NCI-H460 | 46.0 |
| Lung ca. (non-sm. cell) A549 | 0.0 |
| Lung ca. (non-s. cell) NCI-H23 | 8.0 |
| Lung ca. (non-s. cell) HOP-62 | 13.6 |
| Lung ca. (non-s. cl) NCI-H522 | 0.0 |
| Lung ca. (squam.) SW 900 | 18.8 |
| Lung ca. (squam.) NCI-H596 | 0.0 |
| Mammary gland | 1.4 |
| Breast ca.* (pl. ef) MCF-7 | 0.0 |
| Breast ca.* (pl. ef) MDA-MB-231 | 0.0 |
| Breast ca.* (pl. ef) T47D | 1.4 |
| Breast ca. BT-549 | 1.6 |
| Breast ca. MDA-N | 0.0 |
| Ovary | 1.5 |
| Ovarian ca. OVCAR-3 | 1.4 |
| Ovarian ca. OVCAR-4 | 0.0 |
| Ovarian ca. OVCAR-5 | 24.8 |
| Ovarian ca. OVCAR-8 | 0.6 |
| Ovarian ca. IGROV-1 | 0.0 |
| Ovarian ca.* (ascites) SK-OV-3 | 5.4 |
| Uterus | 15.5 |
| Placenta | 1.1 |
| Prostate | 1.4 |
| Prostate ca.* (bone met)PC-3 | 0.4 |
| Testis | 2.3 |
| Melanoma Hs688(A).T | 0.0 |
| Melanoma* (met) Hs688(B).T | 0.0 |
| Melanoma UACC-62 | 0.0 |
| Melanoma M14 | 1.6 |
| Melanoma LOX IMVI | 0.0 |
| Melanoma* (met) SK-MEL-5 | 58.6 |
| Adipose | 1.5 |

Column A - Rel. Exp. (%) Ag3242, Run 165524415

TABLE AWE
Panel 2.2

| Tissue Name | A |
|---|---|
| Normal Colon | 4.4 |
| Colon cancer (OD06064) | 1.8 |
| Colon Margin (OD06064) | 8.9 |
| Colon cancer (OD06159) | 2.0 |
| Colon Margin (OD06159) | 7.1 |
| Colon cancer (OD06297-04) | 2.4 |
| Colon Margin (OD06297-05) | 2.2 |
| CC Gr.2 ascend colon (ODO3921) | 0.9 |
| CC Margin (ODO3921) | 3.8 |
| Colon cancer metastasis (OD06104) | 0.7 |
| Lung Margin (OD06104) | 0.0 |
| Colon mets to lung (OD04451-01) | 1.7 |
| Lung Margin (OD04451-02) | 10.1 |
| Normal Prostate | 0.4 |
| Prostate Cancer (OD04410) | 0.3 |
| Prostate Margin (OD04410) | 0.0 |
| Normal Ovary | 0.0 |
| Ovarian cancer (OD06283-03) | 0.0 |
| Ovarian Margin (OD06283-07) | 0.0 |
| Ovarian Cancer 064008 | 1.9 |
| Ovarian cancer (OD06145) | 0.0 |
| Ovarian Margin (OD06145) | 0.0 |
| Ovarian cancer (OD06455-03) | 0.0 |
| Ovarian Margin (OD06455-07) | 1.2 |
| Normal Lung | 2.3 |
| Invasive poor diff. lung adeno (ODO4945-01 | 1.1 |
| Lung Margin (ODO4945-03) | 1.6 |
| Lung Malignant Cancer (OD03126) | 0.7 |
| Lung Margin (OD03126) | 1.2 |
| Lung Cancer (OD05014A) | 1.2 |
| Lung Margin (OD05014B) | 4.4 |
| Lung cancer (OD06081) | 0.8 |
| Lung Margin (OD06081) | 2.8 |
| Lung Cancer (OD04237-01) | 0.0 |
| Lung Margin (OD04237-02) | 6.0 |
| Ocular Melanoma Metastasis | 100.0 |
| Ocular Melanoma Margin (Liver) | 0.7 |
| Melanoma Metastasis | 1.2 |
| Melanoma Margin (Lung) | 4.9 |
| Normal Kidney | 0.0 |
| Kidney Ca, Nuclear grade 2 (OD04338) | 1.3 |
| Kidney Margin (OD04338) | 0.4 |
| Kidney Ca Nuclear grade 1/2 (OD04339) | 0.6 |
| Kidney Margin (OD04339) | 1.6 |
| Kidney Ca, Clear cell type (OD04340) | 0.0 |
| Kidney Margin (OD04340) | 1.1 |
| Kidney Ca, Nuclear grade 3 (OD04348) | 0.0 |
| Kidney Margin (OD04348) | 1.0 |
| Kidney malignant cancer (OD06204B) | 0.4 |
| Kidney normal adjacent tissue (OD06204E) | 1.8 |
| Kidney Cancer (OD04450-01) | 2.3 |
| Kidney Margin (OD04450-03) | 0.9 |
| Kidney Cancer 8120613 | 0.0 |
| Kidney Margin 8120614 | 0.4 |
| Kidney Cancer 9010320 | 1.4 |
| Kidney Margin 9010321 | 1.3 |
| Kidney Cancer 8120607 | 0.8 |
| Kidney Margin 8120608 | 0.2 |
| Normal Uterus | 5.3 |
| Uterine Cancer 064011 | 0.5 |
| Normal Thyroid | 0.0 |
| Thyroid Cancer 064010 | 0.7 |
| Thyroid Cancer A302152 | 0.9 |
| Thyroid Margin A302153 | 0.0 |
| Normal Breast | 0.6 |
| Breast Cancer (OD04566) | 0.0 |
| Breast Cancer 1024 | 2.1 |
| Breast Cancer (OD04590-01) | 0.0 |
| Breast Cancer Mets (OD04590-03) | 0.0 |
| Breast Cancer Metastasis (OD04655-05) | 0.4 |
| Breast Cancer 064006 | 0.7 |
| Breast Cancer 9100266 | 0.7 |
| Breast Margin 9100265 | 0.2 |
| Breast Cancer A209073 | 0.0 |
| Breast Margin A2090734 | 0.0 |
| Breast cancer (OD06083) | 0.4 |
| Breast cancer node metastasis (OD06083) | 0.9 |
| Normal Liver | 1.3 |
| Liver Cancer 1026 | 0.3 |
| Liver Cancer 1025 | 0.0 |
| Liver Cancer 6004-T | 0.0 |
| Liver Tissue 6004-N | 0.0 |
| Liver Cancer 6005-T | 2.7 |
| Liver Tissue 6005-N | 3.3 |
| Liver Cancer 064003 | 7.1 |
| Normal Bladder | 0.0 |
| Bladder Cancer 1023 | 0.0 |
| Bladder Cancer A302173 | 0.3 |
| Normal Stomach | 5.8 |
| Gastric Cancer 9060397 | 0.0 |
| Stomach Margin 9060396 | 2.6 |
| Gastric Cancer 9060395 | 2.2 |
| Stomach Margin 9060394 | 3.4 |
| Gastric Cancer 064005 | 0.3 |

Column A - Rel. Exp. (%) Ag3242, Run 174443348

TABLE AWF
Panel 4D

| Tissue Name | A | B |
|---|---|---|
| Secondary Th1 act | 0.0 | 0.0 |
| Secondary Th2 act | 0.0 | 0.0 |
| Secondary Tr1 act | 0.0 | 0.0 |
| Secondary Th1 rest | 0.0 | 0.0 |
| Secondary Th2 rest | 0.0 | 0.0 |
| Secondary Tr1 rest | 0.0 | 0.0 |
| Primary Th1 act | 0.0 | 0.0 |
| Primary Th2 act | 0.0 | 0.0 |
| Primary Tr1 act | 0.0 | 0.0 |
| Primary Th1 rest | 0.0 | 0.0 |
| Primary Th2 rest | 0.0 | 0.0 |
| Primary Tr1 rest | 0.0 | 0.0 |
| CD45RA CD4 lymphocyte act | 0.0 | 0.0 |
| CD45RO CD4 lymphocyte act | 0.0 | 0.0 |
| CD8 lymphocyte act | 1.3 | 0.0 |
| Secondary CD8 lymphocyte rest | 0.7 | 0.0 |
| Secondary CD8 lymphocyte act | 0.0 | 0.0 |
| CD4 lymphocyte none | 0.0 | 0.0 |
| 2ry Th1/Th2/Tr1_anti-CD95 CH11 | 0.0 | 0.0 |
| LAK cells rest | 0.0 | 0.0 |
| LAK cells IL-2 | 0.0 | 0.0 |
| LAK cells IL-2 + IL-12 | 0.0 | 0.0 |
| LAK cells IL-2 + IFN gamma | 0.8 | 0.0 |
| LAK cells IL-2 + IL-18 | 0.0 | 0.0 |
| LAK cells PMA/ionomycin | 0.0 | 0.0 |
| NK Cells IL-2 rest | 0.8 | 0.0 |
| Two Way MLR 3 day | 0.0 | 0.0 |
| Two Way MLR 5 day | 0.0 | 0.0 |
| Two Way MLR 7 day | 1.5 | 0.0 |
| PBMC rest | 0.0 | 0.0 |
| PBMC PWM | 0.0 | 0.0 |
| PBMC PHA-L | 0.0 | 0.0 |
| Ramos (B cell) none | 0.0 | 0.0 |
| Ramos (B cell) ionomycin | 0.0 | 0.0 |
| B lymphocytes PWM | 0.0 | 0.0 |
| B lymphocytes CD40L and IL-4 | 0.0 | 0.0 |
| EOL-1 dbcAMP | 0.0 | 0.0 |
| EOL-1 dbcAMP PMA/ionomycin | 0.0 | 0.0 |
| Dendritic cells none | 0.0 | 0.0 |
| Dendritic cells LPS | 0.0 | 0.0 |
| Dendritic cells anti-CD40 | 0.0 | 0.0 |
| Monocytes rest | 0.0 | 0.0 |
| Monocytes LPS | 1.7 | 0.0 |
| Macrophages rest | 0.0 | 0.0 |
| Macrophages LPS | 0.0 | 0.0 |
| HUVEC none | 0.0 | 0.0 |
| HUVEC starved | 0.0 | 0.0 |
| HUVEC IL-1beta | 0.0 | 0.0 |
| HUVEC IFN gamma | 0.4 | 0.0 |
| HUVEC TNF alpha + IFN gamma | 0.0 | 0.0 |
| HUVEC TNF alpha + IL4 | 2.0 | 0.0 |
| HUVEC IL-11 | 0.0 | 0.0 |
| Lung Microvascular EC none | 0.0 | 0.0 |
| Lung Microvascular EC TNFalpha + | 0.0 | 0.0 |
| IL-1beta | | |
| Microvascular Dermal EC none | 0.0 | 0.0 |
| Microsvasular Dermal EC TNFalpha + | 0.0 | 0.0 |
| IL-1beta | | |
| Bronchial epithelium TNFalpha + IL1beta | 0.0 | 0.0 |
| Small airway epithelium none | 0.0 | 0.0 |
| Small airway epithelium TNFalpha + | 6.5 | 11.7 |
| IL-1beta | | |
| Coronery artery SMC rest | 0.7 | 0.0 |
| Coronery artery SMC TNFalpha + IL-1beta | 0.0 | 0.0 |
| Astrocytes rest | 13.9 | 16.8 |
| Astrocytes TNFalpha + IL-1beta | 3.6 | 5.3 |
| KU-812 (Basophil) rest | 0.9 | 1.0 |
| KU-812 (Basophil) PMA/ionomycin | 100.0 | 100.0 |
| CCD1106 (Keratinocytes) none | 0.0 | 0.0 |
| CCD1106 (Keratinocytes) TNFalpha + | 0.0 | 0.0 |
| IL-1beta | | |
| Liver cirrhosis | 1.9 | 3.8 |
| Lupus kidney | 1.3 | 1.4 |
| NCI-H292 none | 42.9 | 48.0 |
| NCI-H292 IL-4 | 14.7 | 14.8 |
| NCI-H292 IL-9 | 40.6 | 36.6 |
| NCI-H292 IL-13 | 12.2 | 7.4 |
| NCI-H292 IFN gamma | 16.8 | 10.9 |
| HPAEC none | 0.0 | 0.0 |
| HPAEC TNF alpha + IL-1 beta | 0.0 | 0.0 |
| Lung fibroblast none | 0.0 | 1.7 |
| Lung fibroblast TNF alpha + IL-1 beta | 0.0 | 0.0 |
| Lung fibroblast IL-4 | 1.7 | 0.0 |
| Lung fibroblast IL-9 | 0.0 | 0.0 |
| Lung fibroblast IL-13 | 0.7 | 0.0 |
| Lung fibroblast IFN gamma | 0.0 | 0.0 |
| Dermal fibroblast CCD1070 rest | 0.0 | 0.0 |
| Dermal fibroblast CCD1070 TNF alpha | 0.0 | 0.0 |
| Dermal fibroblast CCD1070 IL-1 beta | 0.9 | 0.0 |
| Dermal fibroblast IFN gamma | 0.0 | 0.0 |
| Dermal fibroblast IL-4 | 0.0 | 0.0 |
| IBD Colitis 2 | 0.8 | 2.6 |
| IBD Crohn's | 0.0 | 2.0 |
| Colon | 28.1 | 23.2 |
| Lung | 34.9 | 51.4 |
| Thymus | 35.1 | 28.3 |
| Kidney | 8.2 | 7.4 |

Column A - Rel. Exp. (%) Ag1096, Run 160353275
Column B - Rel. Exp. (%) Ag3242, Run 164390548

AI_comprehensive panel_v1.0 Summary: Ag3242 Expression of this gene was ubiquitous in this panel, with high expression in samples derived from patients suffering from ulcerative colitis, Crohns disease and psoriasis (CTs=33). Significant expression was also seen in samples derived from synovium, cartilage and bone of rheumatoid arthritis. Antibody or small molecule therapies designed with the protein encoded for by this gene are useful in the treatment of inflammatory bowel diseases and rheumatoid arthritis.

Panel 1.3D Summary: Ag3242 Highest expression of the CG57448-01 gene was seen in a renal cancer cell line (CT=31.1). Significant expression was also seen in cell lines derived from ovarian cancer, lung cancer, brain cancer and melanoma. Expression of this gene is useful as a marker to differentiate between these samples and other samples on this panel and as a diagnostic marker for the presence of these cancers. This gene encodes a protein that is homologous to cadherin, a cell-adhesion molecule.

Therapeutic modulation of the expression or function of this gene or gene prduct is effective in the treatment of lung, renal and melanoma cancers.

Expression of the this gene was also high in many regions of the brain, including the amygdala, thalamus, cerebellum, and cerebral cortex, with highest expression in the hippocampus. Expression was also detected in the spinal cord. Cadherins can act as axon guidance and cell adhesion proteins, specifically during development and in the response to injury (Ranscht B. (2000) Int. J. Dev. Neurosci. 18: 643-651.). Manipulation of levels of this protein are of use in inducing a compensatory synaptogenic response to neuronal death in Alzheimer's disease, Parkinson's disease, Huntington's disease, spinocerebellar ataxia, progressive supranuclear palsy, ALS, head trauma, stroke, or any other disease/condition associated with neuronal loss.

Among tissues with metabolic function, this gene was moderately expressed in pituitary gland, adrenal gland, thyroid, pancreas, skeletal muscle, and liver, reflecting the widespread role of cadherins in cell-cell adhesion. This expression shows that this gene product plays a role in normal metabolic and neuroendocrine function and that dysregulated expression of this gene contributes to metabolic diseases (such as obesity and diabetes) or neuroendocrine disorders.

Panel 2.2 Summary: Ag3242 Highest expression of this gene was seen inma sample derived from an ocular melanoma metastasis (CT=29). Thus, expression of this gene is useful as a marker to differentiate between this sample and other samples on this panel.

Panel 4D Summary: Ag1096/Ag3242 Highest expression of this gene was detected in the basophil cell line (KU-812) treated with PMA/ionomycin (CTs=30-32). Significant expression was also seen in a cluster of treated and untreated samples derived from the muco-epidermoid cell line NCI-H292. Thus, this gene, which encodes a cadherin homolog, was expressed in both a cell line that is often used as a model of airway epithelium (NCI-H292) and a cell line that is a reasonable model for the inflammatory cells that contribute to various inflammatory lung diseases. Therapeutic modulation of this gene or gene prodcut may is useful to reduce or eliminate the symptoms of patients suffering from pathological and inflammatory lung disorders, including chronic obstructive pulmonary disease, asthma, allergy and emphysema.

Low but significant levels of expression were also seen in the samples derived from normal colon, kidney, lung and thymus. Therapeutic modulation of the expression or function of this gene or gene product is important for maintaining or restoring normal function to this organs during inflammation.

AX. CG5740905 and CG57409-07: Protein Containing MAM and Ig Domains

Expression of gene CG57409-05 and full length physical clone CG57409-07 was assessed using the primer-probe sets Ag5267 and Ag6188, described in Tables AXA and AXB. Results of the RTQ-PCR runs are shown in Tables AXC, AXD and AXE.

TABLE AXA
Probe Name Ag5267

| | | | | SEQ |
|---|---|---|---|---|
| | | | Start | ID |
| Primers | Sequences | Length | Position | No |
| Forward | 5′-gcggtcccggaaca-3′ | 14 | 1166 | 1528 |
| Probe | TET-5′-cacgcctggtctctcagtggca-3′-TAMRA | 22 | 1197 | 1529 |
| Reverse | 5′-gcctgctgccacacatt-3′ | 17 | 1227 | 1530 |

TABLE AXB
Probe Name Ag6188

| | | | | SEQ |
|---|---|---|---|---|
| | | | Start | ID |
| Primers | Sequences | Length | Position | No |
| Forward | 5′-ataaagtggtggtgatgcc-3′ | 19 | 1381 | 1531 |
| Probe | TET-5′-agtggagccccctgccagtc-3′-TAMRA | 20 | 1404 | 1532 |
| Reverse | 5′-gaggaagatggccatg-3′ | 16 | 1445 | 1533 |

TABLE AXC
General_screening_panel_v1.5

| Tissue Name | A |
|---|---|
| Adipose | 1.5 |
| Melanoma* Hs688(A).T | 4.4 |
| Melanoma* Hs688(B).T | 7.7 |
| Melanoma* M14 | 0.2 |
| Melanoma* LOXIMVI | 0.3 |
| Melanoma* SK-MEL-5 | 9.2 |
| Squamous cell carcinoma SCC-4 | 0.0 |
| Testis Pool | 0.7 |
| Prostate ca.* (bone met) PC-3 | 0.0 |
| Prostate Pool | 2.0 |
| Placenta | 0.0 |
| Uterus Pool | 2.1 |
| Ovarian ca. OVCAR-3 | 0.4 |
| Ovarian ca. SK-OV-3 | 7.0 |
| Ovarian ca. OVCAR-4 | 1.8 |
| Ovarian ca. OVCAR-5 | 2.2 |
| Ovarian ca. IGROV-1 | 0.5 |
| Ovarian ca. OVCAR-8 | 0.7 |
| Ovary | 4.3 |
| Breast ca. MCF-7 | 0.0 |
| Breast ca. MDA-MB-231 | 3.5 |
| Breast ca. BT 549 | 7.5 |
| Breast ca. T47D | 2.5 |
| Breast ca. MDA-N | 2.6 |
| Breast Pool | 5.7 |
| Trachea | 0.5 |
| Lung | 2.5 |
| Fetal Lung | 4.3 |
| Lungca. NCI-N417 | 0.6 |
| Lung ca. LX-1 | 3.6 |
| Lung ca. NCI-H146 | 0.0 |
| Lung ca. SHP-77 | 0.3 |
| Lung ca. A549 | 2.6 |
| Lung ca. NCI-H526 | 0.2 |
| Lung ca. NCI-H23 | 0.1 |
| Lung ca. NCI-H460 | 0.2 |
| Lung ca. HOP-62 | 1.3 |
| Lung ca. NCI-H522 | 19.3 |
| Liver | 0.1 |
| Fetal Liver | 0.3 |
| Liver ca. HepG2 | 0.0 |
| Kidney Pool | 13.3 |
| Fetal Kidney | 1.2 |
| Renal ca. 786-0 | 0.3 |
| Renal ca. A498 | 5.2 |
| Renal ca. ACHN | 6.1 |
| Renal ca. UO-31 | 2.5 |
| Renal ca. TK-10 | 3.4 |
| Bladder | 1.0 |
| Gastric ca. (liver met.) NCI-N87 | 0.0 |
| Gastric ca. KATO III | 0.0 |
| Colon ca. SW-948 | 0.0 |
| Colon ca. SW480 | 3.5 |
| Colon ca.* (SW480 met) SW620 | 0.3 |
| Colon ca. HT29 | 0.0 |
| Colon ca. HCT-116 | 2.6 |
| Colon ca. CaCo-2 | 0.1 |
| Colon cancer tissue | 0.9 |
| Colon ca. SW1116 | 0.2 |
| Colon ca. Colo-205 | 0.0 |
| Colon ca. SW-48 | 0.0 |
| Colon Pool | 7.3 |
| Small Intestine Pool | 8.7 |
| Stomach Pool | 2.5 |
| Bone Marrow Pool | 3.6 |
| Fetal Heart | 1.5 |
| Heart Pool | 3.5 |
| Lymph Node Pool | 6.2 |
| Fetal Skeletal Muscle | 4.9 |
| Skeletal Muscle Pool | 1.0 |
| Spleen Pool | 2.1 |
| Thymus Pool | 2.9 |
| CNS cancer (glio/astro) U87-MG | 1.3 |
| CNS cancer (glio/astro) U-118-MG | 7.2 |
| CNS cancer (neuro; met) SK-N-AS | 0.3 |
| CNS cancer (astro) SF-539 | 0.6 |
| CNS cancer (astro) SNB-75 | 27.0 |
| CNS cancer (glio) SNB-19 | 0.5 |
| CNS cancer (glio) SF-295 | 4.2 |
| Brain (Amygdala) Pool | 2.3 |
| Brain (cerebellum) | 100.0 |
| Brain (fetal) | 34.4 |
| Brain (Hippocampus) Pool | 3.0 |
| Cerebral Cortex Pool | 3.5 |
| Brain (Substantia nigra) Pool | 3.2 |
| Brain (Thalamus) Pool | 2.7 |
| Brain (whole) | 5.1 |
| Spinal Cord Pool | 5.4 |
| Adrenal Gland | 4.0 |
| Pituitary gland Pool | 2.0 |
| Salivary Gland | 0.2 |
| Thyroid (female) | 0.3 |
| Pancreatic ca. CAPAN2 | 0.0 |
| Pancreas Pool | 2.5 |

Column A - Rel. Exp. (%) Ag6188, Run 257761488

TABLE AXD
Panel 4.1D

| Tissue Name | A |
|---|---|
| Secondary Th1 act | 0.0 |
| Secondary Th2 act | 4.3 |
| Secondary Tr1 act | 3.4 |
| Secondary Th1 rest | 0.6 |
| Secondary Th2 rest | 2.1 |
| Secondary Tr1 rest | 0.8 |
| Primary Th1 act | 0.0 |
| Primary Th2 act | 0.6 |
| Primary Tr1 act | 0.8 |
| Primary Th1 rest | 0.2 |
| Primary Th2 rest | 0.3 |
| Primary Tr1 rest | 0.2 |
| CD45RA CD4 lymphocyte act | 1.6 |
| CD45RO CD4 lymphocyte act | 3.4 |
| CD8 lymphocyte act | 0.4 |
| Secondary CD8 lymphocyte rest | 0.1 |
| Secondary CD8 lymphocyte act | 0.2 |
| CD4 lymphocyte none | 0.9 |
| 2ry Th1/Th2/Tr1_anti-CD95 CH11 | 1.8 |
| LAK cells rest | 6.2 |
| LAK cells IL-2 | 3.3 |
| LAK cells IL-2 + IL-12 | 1.2 |
| LAK cells IL-2 + IFN gamma | 0.7 |
| LAK cells IL-2 + IL-18 | 0.6 |
| LAK cells PMA/ionomycin | 41.8 |
| NK Cells IL-2 rest | 9.0 |
| Two Way MLR 3 day | 2.9 |
| Two Way MLR 5 day | 3.5 |
| Two Way MLR 7 day | 2.7 |
| PBMC rest | 0.3 |
| PBMC PWM | 0.0 |
| PBMC PHA-L | 1.2 |
| Ramos (B cell) none | 0.0 |
| Ramos (B cell) ionomycin | 0.0 |
| B lymphocytes PWM | 0.7 |
| B lymphocytes CD40L and IL-4 | 7.6 |
| EOL-1 dbcAMP | 7.7 |
| EOL-1 dbcAMP PMA/ionomycin | 100.0 |
| Dendritic cells none | 0.5 |
| Dendritic cells LPS | 0.4 |
| Dendritic cells anti-CD40 | 0.4 |
| Monocytes rest | 0.0 |
| Monocytes LPS | 3.0 |
| Macrophages rest | 0.0 |
| Macrophages LPS | 0.7 |
| HUVEC none | 0.7 |
| HUVEC starved | 1.2 |
| HUVEC IL-1beta | 1.4 |
| HUVEC IFN gamma | 4.8 |
| HUVEC TNF alpha + IFN gamma | 3.8 |
| HUVEC TNF alpha + IL4 | 0.4 |
| HUVEC IL-11 | 0.4 |
| Lung Microvascular EC none | 0.0 |
| Lung Microvascular EC TNFalpha + IL-1beta | 0.4 |
| Microvascular Dermal EC none | 0.0 |
| Microsvasular Dermal EC TNFalpha + IL-1beta | 0.2 |
| Bronchial epithelium TNFalpha + IL1beta | 0.7 |
| Small airway epithelium none | 0.2 |
| Small airway epithelium TNFalpha + IL-1beta | 1.6 |
| Coronery artery SMC rest | 0.9 |
| Coronery artery SMC TNFalpha + IL-1beta | 1.9 |
| Astrocytes rest | 8.8 |
| Astrocytes TNFalpha + IL-1beta | 11.0 |
| KU-812 (Basophil) rest | 1.4 |
| KU-812 (Basophil) PMA/ionomycin | 9.3 |
| CCD1106 (Keratinocytes) none | 4.8 |
| CCD1106 (Keratinocytes) TNFalpha + IL-1beta | 1.2 |
| Liver cirrhosis | 2.0 |
| NCI-H292 none | 0.4 |
| NCI-H292 IL-4 | 1.8 |
| NCI-H292 IL-9 | 1.4 |
| NCI-H292 IL-13 | 2.8 |
| NCI-H292 IFN gamma | 5.6 |
| HPAEC none | 0.2 |
| HPAEC TNF alpha + IL-1 beta | 4.9 |
| Lung fibroblast none | 46.7 |
| Lung fibroblast TNF alpha + IL-1 beta | 9.7 |
| Lung fibroblast IL-4 | 23.5 |
| Lung fibroblast IL-9 | 32.5 |
| Lung fibroblast IL-13 | 18.6 |
| Lung fibroblast IFN gamma | 73.7 |
| Dermal fibroblast CCD1070 rest | 1.8 |
| Dermal fibroblast CCD1070 TNF alpha | 0.3 |
| Dermal fibroblast CCD1070 IL-1 beta | 2.0 |
| Dermal fibroblast IFN gamma | 11.7 |
| Dermal fibroblast IL-4 | 4.0 |
| Dermal Fibroblasts rest | 10.1 |
| Neutrophils TNFa + LPS | 0.0 |
| Neutrophils rest | 0.7 |
| Colon | 0.6 |
| Lung | 0.7 |
| Thymus | 1.1 |
| Kidney | 2.4 |

Column A - Rel. Exp. (%) Ag5267, Run 230510063

TABLE AXE
general oncology screening panel_v_2.4

| Tissue Name | A |
|---|---|
| Colon cancer 1 | 6.4 |
| Colon cancer NAT 1 | 0.0 |
| Colon cancer 2 | 2.7 |
| Colon cancer NAT 2 | 3.9 |
| Colon cancer 3 | 7.0 |
| Colon cancer NAT 3 | 11.5 |
| Colon malignant cancer 4 | 5.5 |
| Colon normal adjacent tissue 4 | 3.7 |
| Lung cancer 1 | 7.7 |
| Lung NAT 1 | 0.5 |
| Lung cancer 2 | 12.3 |
| Lung NAT 2 | 3.2 |
| Squamous cell carcinoma 3 | 48.6 |
| Lung NAT 3 | 4.5 |
| metastatic melanoma 1 | 52.1 |
| Melanoma 2 | 23.8 |
| Melanoma 3 | 3.4 |
| metastatic melanoma 4 | 97.9 |
| metastatic melanoma 5 | 100.0 |
| Bladder cancer 1 | 4.5 |
| Bladder cancer NAT 1 | 0.0 |
| Bladder cancer 2 | 19.5 |
| Bladder cancer NAT 2 | 1.4 |
| Bladder cancer NAT 3 | 0.0 |
| Bladder cancer NAT 4 | 20.9 |
| Prostate adenocarcinoma 1 | 69.3 |
| Prostate adenocarcinoma 2 | 6.5 |
| Prostate adenocarcinoma 3 | 17.7 |
| Prostate adenocarcinoma 4 | 11.7 |
| Prostate cancer NAT 5 | 5.0 |
| Prostate adenocarcinoma 6 | 13.4 |
| Prostate adenocarcinoma 7 | 10.3 |
| Prostate adenocarcinoma 8 | 11.3 |
| Prostate adenocarcinoma 9 | 93.3 |
| Prostate cancer NAT 10 | 1.4 |
| Kidney cancer 1 | 14.8 |
| KidneyNAT 1 | 2.9 |
| Kidney cancer 2 | 16.4 |
| Kidney NAT 2 | 12.2 |
| Kidney cancer 3 | 18.4 |
| Kidney NAT 3 | 5.3 |
| Kidney cancer 4 | 2.8 |
| Kidney NAT 4 | 2.1 |

Column A - Rel. Exp. (%) Ag6188, Run 264979750

General_screening_panel_v1.5 Summary: Ag6188 Significant expression of this gene was seen in the brain. Expression of this gene is useful as a marker to differentiate between brain derived samples and other samples on this panel.

Among tissues with metabolic function, this gene was expressed at moderate to low levels in pituitary, adipose, adrenal gland, pancreas, thyroid, and adult and fetal skeletal muscle, heart, and liver. This widespread expression among these tissues shows that this gene product plays a role in normal neuroendocrine and metabolic function and that disregulated expression of this gene contributes to neuroendocrine disorders or metabolic diseases, such as obesity and diabetes.

Panel 4.1D Summary: Ag5267 This transcript was expressed in LAK cells and treatment of the LAK cells with PMA and ionomycin upregulated the expression of this transcript. This transcript was also induced in activated EOL cells and in fibroblasts. This gene encodes a putative NCAM, a type of cell surface protein often involved in cellular interaction, adhesion and signaling. Therapeutics designed with the protein encoded for this transcript are important in the treatment of diseases such as asthma, emphysema, psoriasis and arthritis.

general oncology screening panel_V_{—}2.4 Summary: Ag6188 Highest expression of this gene was seen in two melanoma samples (CTs=31). Prominent expression was seen in a squamous cell carcinoma sample and two prostate adenocarcinoma samples. Targeting this gene or its protein product is useful in the treatment of these cancers.

AY. CG57448-01: Protocadherin 10

Expression of gene CG57448-01 was assessed using the primer-probe sets Ag1096, and Ag3242, described in Tables AYA and AYB. Results of the RTQ-PCR runs are shown in Tables AYC, AYD, AYE, AYF, and AYG.

TABLE AYA
Probe Name Ag1096

| | | | | SEQ |
|---|---|---|---|---|
| | | | Start | ID |
| Primers | Sequences | Length | Position | No |
| Forward | 5′-actttggaagaggcattgct-3′ | 20 | 2455 | 1534 |
| Probe | TET-5′-cagacagattatgggctccatcgctt-3′- | 26 | 2476 | 1535 |
| | TAMRA | | | |
| Reverse | 5′-ctcgggataaccatgatcact-3′ | 21 | 2515 | 1536 |

TABLE AYB
Probe Name Ag3242

| | | | | |
|---|---|---|---|---|
| | | | | SEQ |
| | | | Start | ID |
| Primers | Sequences | Length | Position | No |

| | | | | |
|---|---|---|---|---|
| Forward | 5′-gggaccaatgctcaaattactt-3′ | 22 | 1090 | 1537 |
| Probe | TET-5′-tcagaaagttccacaagcatctaagga-3′- | 27 | 1122 | 1538 |
| | TAMRA | | | |
| Reverse | 5′-atgactccagtgttttcatcca-3′ | 22 | 1160 | 1539 |

TABLE AYC
AI_comprehensive panel_v1.0

| Tissue Name | A |
|---|---|
| 110967 COPD-F | 28.9 |
| 110980 COPD-F | 6.7 |
| 110968 COPD-M | 19.9 |
| 110977 COPD-M | 8.4 |
| 110989 Emphysema-F | 49.3 |
| 110992 Emphysema-F | 22.8 |
| 110993 Emphysema-F | 31.0 |
| 110994 Emphysema-F | 12.7 |
| 110995 Emphysema-F | 25.5 |
| 110996 Emphysema-F | 15.8 |
| 110997 Asthma-M | 7.5 |
| 111001 Asthma-F | 30.4 |
| 111002 Asthma-F | 45.4 |
| 111003 Atopic Asthma-F | 45.1 |
| 111004 Atopic Asthma-F | 22.4 |
| 111005 Atopic Asthma-F | 25.3 |
| 111006 Atopic Asthma-F | 13.9 |
| 111417 Allergy-M | 15.1 |
| 112347 Allergy-M | 5.0 |
| 112349 Normal Lung-F | 1.9 |
| 112357 Normal Lung-F | 5.1 |
| 112354 Normal Lung-M | 11.6 |
| 112374 Crohns-F | 32.5 |
| 112389 Match Control Crohns-F | 14.4 |
| 112375 Crohns-F | 36.1 |
| 112732 Match Control Crohns-F | 0.0 |
| 112725 Crohns-M | 45.4 |
| 112387 Match Control Crohns-M | 18.3 |
| 112378 Crohns-M | 3.8 |
| 112390 Match Control Crohns-M | 22.4 |
| 112726 Crohns-M | 45.7 |
| 112731 Match Control Crohns-M | 12.6 |
| 112380 Ulcer Col-F | 28.3 |
| 112734 Match Control Ulcer Col-F | 0.8 |
| 112384 Ulcer Col-F | 100.0 |
| 112737 Match Control Ulcer Col-F | 28.1 |
| 112386 Ulcer Col-F | 25.5 |
| 112738 Match Control Ulcer Col-F | 2.1 |
| 112381 Ulcer Col-M | 2.3 |
| 112735 Match Control Ulcer Col-M | 53.6 |
| 112382 Ulcer Col-M | 15.3 |
| 112394 Match Control Ulcer Col-M | 10.2 |
| 112383 Ulcer Col-M | 45.4 |
| 112736 Match Control Ulcer Col-M | 4.0 |
| 112423 Psoriasis-F | 90.1 |
| 112427 Match Control Psoriasis-F | 55.9 |
| 112418 Psoriasis-M | 25.7 |
| 112723 Match Control Psoriasis-M | 0.0 |
| 112419 Psoriasis-M | 66.0 |
| 112424 Match Control Psoriasis-M | 42.0 |
| 112420 Psoriasis-M | 44.4 |
| 112425 Match Control Psoriasis-M | 24.3 |
| 104689 (MF) OA Bone-Backus | 81.2 |
| 104690 (MF) Adj “Normal” Bone-Backus | 16.6 |
| 104691 (MF) OA Synovium-Backus | 1.5 |
| 104692 (BA) OA Cartilage-Backus | 7.4 |
| 104694 (BA) OA Bone-Backus | 56.3 |
| 104695 (BA) Adj “Normal” Bone-Backus | 31.6 |
| 104696 (BA) OA Synovium-Backus | 0.0 |
| 104700 (SS) OA Bone-Backus | 63.7 |
| 104701 (SS) Adj “Normal” Bone-Backus | 63.3 |
| 104702 (SS) OA Synovium-Backus | 6.1 |
| 117093 OA Cartilage Rep7 | 16.3 |
| 112672 OA Bone5 | 13.2 |
| 112673 OA Synovium5 | 5.4 |
| 112674 OA Synovial Fluid cells5 | 7.7 |
| 117100 OA Cartilage Rep14 | 4.5 |
| 112756 OA Bone9 | 17.1 |
| 112757 OA Synovium9 | 21.8 |
| 112758 OA Synovial Fluid Cells9 | 13.3 |
| 117125 RA Cartilage Rep2 | 28.1 |
| 113492 Bone2 RA | 34.2 |
| 113493 Synovium2 RA | 8.9 |
| 113494 Syn Fluid Cells RA | 25.7 |
| 113499 Cartilage4 RA | 45.1 |
| 113500 Bone4 RA | 47.3 |
| 113501 Synovium4 RA | 42.6 |
| 113502 Syn Fluid Cells4 RA | 25.5 |
| 113495 Cartilage3 RA | 33.7 |
| 113496 Bone3 RA | 38.7 |
| 113497 Synovium3 RA | 18.2 |
| 113498 Syn Fluid Cells3 RA | 30.8 |
| 117106 Normal Cartilage Rep20 | 0.0 |
| 113663 Bone3 Normal | 2.8 |
| 113664 Synovium3 Normal | 0.0 |
| 113665 Syn Fluid Cells3 Normal | 2.9 |
| 117107 Normal Cartilage Rep22 | 10.5 |
| 113667 Bone4 Normal | 17.2 |
| 113668 Synovium4 Normal | 17.0 |
| 113669 Syn Fluid Cells4 Normal | 36.3 |

Column A - Rel. Exp. (%) Ag3242, Run 253058919

TABLE AYD
CNS_neurodegeneration_v1.0

| Tissue Name | A | B |
|---|---|---|
| AD 1 Hippo | 74.2 | 45.4 |
| AD 2 Hippo | 18.8 | 27.5 |
| AD 3 Hippo | 32.3 | 19.5 |
| AD 4 Hippo | 6.8 | 6.3 |
| AD 5 Hippo | 39.2 | 30.8 |
| AD 6 Hippo | 100.0 | 100.0 |
| Control 2 Hippo | 89.5 | 61.6 |
| Control 4 Hippo | 13.6 | 10.1 |
| Control (Path) 3 Hippo | 17.6 | 3.8 |
| AD 1 Temporal Ctx | 8.2 | 6.2 |
| AD 2 Temporal Ctx | 19.8 | 11.8 |
| AD 3 Temporal Ctx | 9.9 | 4.0 |
| AD 4 Temporal Ctx | 12.1 | 6.3 |
| AD 5 Inf Temporal Ctx | 59.5 | 47.3 |
| AD 5 Sup Temporal Ctx | 72.7 | 67.8 |
| AD 6 Inf Temporal Ctx | 12.9 | 12.9 |
| AD 6 Sup Temporal Ctx | 14.6 | 12.2 |
| Control 1 Temporal Ctx | 7.5 | 8.5 |
| Control 2 Temporal Ctx | 34.4 | 35.1 |
| Control 3 Temporal Ctx | 13.9 | 10.8 |
| Control 3 Temporal Ctx | 5.2 | 2.9 |
| Control (Path) 1 Temporal Ctx | 38.7 | 32.8 |
| Control (Path) 2 Temporal Ctx | 25.7 | 18.3 |
| Control (Path) 3 Temporal Ctx | 4.1 | 6.0 |
| Control (Path) 4 Temporal Ctx | 18.4 | 7.6 |
| AD 1 Occipital Ctx | 14.8 | 7.0 |
| AD 2 Occipital Ctx (Missing) | 0.6 | 1.9 |
| AD 3 Occipital Ctx | 23.2 | 11.5 |
| AD 4 Occipital Ctx | 12.8 | 8.4 |
| AD 5 Occipital Ctx | 34.6 | 23.3 |
| AD 6 Occipital Ctx | 10.7 | 2.1 |
| Control 1 Occipital Ctx | 4.0 | 1.6 |
| Control 2 Occipital Ctx | 20.6 | 23.5 |
| Control 3 Occipital Ctx | 6.7 | 7.7 |
| Control 4 Occipital Ctx | 5.1 | 2.8 |
| Control (Path) 1 Occipital Ctx | 30.4 | 26.4 |
| Control (Path) 2 Occipital Ctx | 6.5 | 1.9 |
| Control (Path) 3 Occipital Ctx | 4.4 | 2.6 |
| Control (Path) 4 Occipital Ctx | 7.1 | 3.2 |
| Control 1 Parietal Ctx | 4.6 | 4.8 |
| Control 2 Parietal Ctx | 27.0 | 24.3 |
| Control 3 Parietal Ctx | 14.5 | 14.0 |
| Control (Path) 1 Parietal Ctx | 40.3 | 40.3 |
| Control (Path) 2 Parietal Ctx | 17.2 | 7.8 |
| Control (Path) 3 Parietal Ctx | 7.0 | 2.7 |
| Control (Path) 4 Parietal Ctx | 21.8 | 20.0 |

Column A - Rel. Exp. (%) Ag1096, Run 206231465
Column B - Rel. Exp. (%) Ag3242, Run 206533581

TABLE AYE
Panel 1.3D

| Tissue Name | A |
|---|---|
| Liver adenocarcinoma | 0.0 |
| Pancreas | 1.0 |
| Pancreatic ca. CAPAN 2 | 0.0 |
| Adrenal gland | 0.3 |
| Thyroid | 0.0 |
| Salivary gland | 0.6 |
| Pituitary gland | 4.8 |
| Brain (fetal) | 11.8 |
| Brain (whole) | 40.6 |
| Brain (amygdala) | 31.6 |
| Brain (cerebellum) | 3.0 |
| Brain (hippocampus) | 76.3 |
| Brain (substantia nigra) | 2.1 |
| Brain (thalamus) | 6.3 |
| Cerebral Cortex | 27.7 |
| Spinal cord | 7.7 |
| glio/astro U87-MG | 0.6 |
| glio/astro U-118-MG | 0.5 |
| astrocytoma SW1783 | 0.0 |
| neuro*; met SK-N-AS | 7.5 |
| astrocytoma SF-539 | 0.0 |
| astrocytoma SNB-75 | 8.4 |
| glioma SNB-19 | 3.0 |
| glioma U251 | 66.4 |
| glioma SF-295 | 0.6 |
| Heart (fetal) | 3.7 |
| Heart | 1.7 |
| Skeletal muscle (fetal) | 6.9 |
| Skeletal muscle | 1.9 |
| Bone marrow | 0.0 |
| Thymus | 0.4 |
| Spleen | 0.0 |
| Lymph node | 0.0 |
| Colorectal | 3.5 |
| Stomach | 2.5 |
| Small intestine | 7.2 |
| Colon ca. SW480 | 0.0 |
| Colon ca.* SW620(SW480 met) | 0.0 |
| Colon ca. HT29 | 0.0 |
| Colon ca. HCT-116 | 0.0 |
| Colon ca. CaCo-2 | 0.0 |
| Colon ca. tissue(ODO3866) | 0.0 |
| Colon ca. HCC-2998 | 2.1 |
| Gastric ca.* (liver met) NCI-N87 | 0.0 |
| Bladder | 0.0 |
| Trachea | 9.0 |
| Kidney | 1.5 |
| Kidney (fetal) | 2.7 |
| Renal ca. 786-0 | 0.0 |
| Renal ca. A498 | 0.0 |
| Renal ca. RXF 393 | 0.4 |
| Renal ca. ACHN | 100.0 |
| Renal ca. UO-31 | 0.0 |
| Renal ca. TK-10 | 1.1 |
| Liver | 1.0 |
| Liver (fetal) | 0.0 |
| Liver ca. (hepatoblast) HepG2 | 0.0 |
| Lung | 4.8 |
| Lung (fetal) | 2.2 |
| Lung ca. (small cell) LX-1 | 3.7 |
| Lung ca. (small cell) NCI-H69 | 0.0 |
| Lung ca. (s. cell var.) SHP-77 | 16.0 |
| Lung ca. (large cell)NCI-H460 | 46.0 |
| Lung ca. (non-sm. cell) A549 | 0.0 |
| Lung ca. (non-s. cell) NCI-H23 | 8.0 |
| Lung ca. (non-s. cell) HOP-62 | 13.6 |
| Lung ca. (non-s. cl) NCI-H522 | 0.0 |
| Lung ca. (squam.) SW 900 | 18.8 |
| Lung ca. (squam.) NCI-H596 | 0.0 |
| Mammary gland | 1.4 |
| Breast ca.* (pl. ef) MCF-7 | 0.0 |
| Breast ca.* (pl. ef) MDA-MB-231 | 0.0 |
| Breast ca.* (pl. ef) T47D | 1.4 |
| Breast ca. BT-549 | 1.6 |
| Breast ca. MDA-N | 0.0 |
| Ovary | 1.5 |
| Ovarian ca. OVCAR-3 | 1.4 |
| Ovarian ca. OVCAR-4 | 0.0 |
| Ovarian ca. OVCAR-5 | 24.8 |
| Ovarian ca. OVCAR-8 | 0.6 |
| Ovarian ca. IGROV-1 | 0.0 |
| Ovarian ca.* (ascites) SK-OV-3 | 5.4 |
| Uterus | 15.5 |
| Placenta | 1.1 |
| Prostate | 1.4 |
| Prostate ca.* (bone met)PC-3 | 0.4 |
| Testis | 2.3 |
| Melanoma Hs688(A).T | 0.0 |
| Melanoma* (met) Hs688(B).T | 0.0 |
| Melanoma UACC-62 | 0.0 |
| Melanoma M14 | 1.6 |
| Melanoma LOX IMVI | 0.0 |
| Melanoma* (met) SK-MEL-5 | 58.6 |
| Adipose | 1.5 |

Column A - Rel. Exp. (%) Ag3242, Run 165524415

TABLE AYF
Panel 2.2

| Tissue Name | A |
|---|---|
| Normal Colon | 4.4 |
| Colon cancer (OD06064) | 1.8 |
| Colon Margin (OD06064) | 8.9 |
| Colon cancer (OD06159) | 2.0 |
| Colon Margin (OD06159) | 7.1 |
| Colon cancer (OD06297-04) | 2.4 |
| Colon Margin (OD06297-05) | 2.2 |
| CC Gr.2 ascend colon (ODO3921) | 0.9 |
| CC Margin (ODO3921) | 3.8 |
| Colon cancer metastasis (OD06104) | 0.7 |
| Lung Margin (OD06104) | 0.0 |
| Colon mets to lung (OD04451-01) | 1.7 |
| Lung Margin (OD04451-02) | 10.1 |
| Normal Prostate | 0.4 |
| Prostate Cancer (OD04410) | 0.3 |
| Prostate Margin (OD04410) | 0.0 |
| Normal Ovary | 0.0 |
| Ovarian cancer (OD06283-03) | 0.0 |
| Ovarian Margin (OD06283-07) | 0.0 |
| Ovarian Cancer 064008 | 1.9 |
| Ovarian cancer (OD06145) | 0.0 |
| Ovarian Margin (OD06145) | 0.0 |
| Ovarian cancer (OD06455-03) | 0.0 |
| Ovarian Margin (OD06455-07) | 1.2 |
| Normal Lung | 2.3 |
| Invasive poor diff. lung adeno (ODO4945-01 | 1.1 |
| Lung Margin (ODO4945-03) | 1.6 |
| Lung Malignant Cancer (OD03126) | 0.7 |
| Lung Margin (OD03126) | 1.2 |
| Lung Cancer (OD05014A) | 1.2 |
| Lung Margin (OD05014B) | 4.4 |
| Lung cancer (OD06081) | 0.8 |
| Lung Margin (OD06081) | 2.8 |
| Lung Cancer (OD04237-01) | 0.0 |
| Lung Margin (OD04237-02) | 6.0 |
| Ocular Melanoma Metastasis | 100.0 |
| Ocular Melanoma Margin (Liver) | 0.7 |
| Melanoma Metastasis | 1.2 |
| Melanoma Margin (Lung) | 4.9 |
| Normal Kidney | 0.0 |
| Kidney Ca, Nuclear grade 2 (OD04338) | 1.3 |
| Kidney Margin (OD04338) | 0.4 |
| Kidney Ca Nuclear grade 1/2 (OD04339) | 0.6 |
| Kidney Margin (OD04339) | 1.6 |
| Kidney Ca, Clear cell type (OD04340) | 0.0 |
| Kidney Margin (OD04340) | 1.1 |
| Kidney Ca, Nuclear grade 3 (OD04348) | 0.0 |
| Kidney Margin (OD04348) | 1.0 |
| Kidney malignant cancer (OD06204B) | 0.4 |
| Kidney normal adjacent tissue (OD06204E) | 1.8 |
| Kidney Cancer (OD04450-01) | 2.3 |
| Kidney Margin (OD04450-03) | 0.9 |
| Kidney Cancer 8120613 | 0.0 |
| Kidney Margin 8120614 | 0.4 |
| Kidney Cancer 9010320 | 1.4 |
| Kidney Margin 9010321 | 1.3 |
| Kidney Cancer 8120607 | 0.8 |
| Kidney Margin 8120608 | 0.2 |
| Normal Uterus | 5.3 |
| Uterine Cancer 064011 | 0.5 |
| Normal Thyroid | 0.0 |
| Thyroid Cancer 064010 | 0.7 |
| Thyroid Cancer A302152 | 0.9 |
| Thyroid Margin A302153 | 0.0 |
| Normal Breast | 0.6 |
| Breast Cancer (OD04566) | 0.0 |
| Breast Cancer 1024 | 2.1 |
| Breast Cancer (OD04590-01) | 0.0 |
| Breast Cancer Mets (OD04590-03) | 0.0 |
| Breast Cancer Metastasis (OD04655-05) | 0.4 |
| Breast Cancer 064006 | 0.7 |
| Breast Cancer 9100266 | 0.7 |
| Breast Margin 9100265 | 0.2 |
| Breast Cancer A209073 | 0.0 |
| Breast Margin A2090734 | 0.0 |
| Breast cancer (OD06083) | 0.4 |
| Breast cancer node metastasis (OD06083) | 0.9 |
| Normal Liver | 1.3 |
| Liver Cancer 1026 | 0.3 |
| Liver Cancer 1025 | 0.0 |
| Liver Cancer 6004-T | 0.0 |
| Liver Tissue 6004-N | 0.0 |
| Liver Cancer 6005-T | 2.7 |
| Liver Tissue 6005-N | 3.3 |
| Liver Cancer 064003 | 7.1 |
| Normal Bladder | 0.0 |
| Bladder Cancer 1023 | 0.0 |
| Bladder Cancer A302173 | 0.3 |
| Normal Stomach | 5.8 |
| Gastric Cancer 9060397 | 0.0 |
| Stomach Margin 9060396 | 2.6 |
| Gastric Cancer 9060395 | 2.2 |
| Stomach Margin 9060394 | 3.4 |
| Gastric Cancer 064005 | 0.3 |

Column A - Rel. Exp. (%) Ag3242, Run 174443348

TABLE AYG
Panel 4D

| Tissue Name | A | B |
|---|---|---|
| Secondary Th1 act | 0.0 | 0.0 |
| Secondary Th2 act | 0.0 | 0.0 |
| Secondary Tr1 act | 0.0 | 0.0 |
| Secondary Th1 rest | 0.0 | 0.0 |
| Secondary Th2 rest | 0.0 | 0.0 |
| Secondary Tr1 rest | 0.0 | 0.0 |
| Primary Th1 act | 0.0 | 0.0 |
| Primary Th2 act | 0.0 | 0.0 |
| Primary Tr1 act | 0.0 | 0.0 |
| Primary Th1 rest | 0.0 | 0.0 |
| Primary Th2 rest | 0.0 | 0.0 |
| Primary Tr1 rest | 0.0 | 0.0 |
| CD45RA CD4 lymphocyte act | 0.0 | 0.0 |
| CD45RO CD4 lymphocyte act | 0.0 | 0.0 |
| CD8 lymphocyte act | 1.3 | 0.0 |
| Secondary CD8 lymphocyte rest | 0.7 | 0.0 |
| Secondary CD8 lymphocyte act | 0.0 | 0.0 |
| CD4 lymphocyte none | 0.0 | 0.0 |
| 2ry Th1/Th2/Tr1_anti-CD95 CH11 | 0.0 | 0.0 |
| LAK cells rest | 0.0 | 0.0 |
| LAK cells IL-2 | 0.0 | 0.0 |
| LAK cells IL-2 + IL-12 | 0.0 | 0.0 |
| LAK cells IL-2 + IFN gamma | 0.8 | 0.0 |
| LAK cells IL-2 + IL-18 | 0.0 | 0.0 |
| LAK cells PMA/ionomycin | 0.0 | 0.0 |
| NK Cells IL-2 rest | 0.8 | 0.0 |
| Two Way MLR 3 day | 0.0 | 0.0 |
| Two Way MLR 5 day | 0.0 | 0.0 |
| Two Way MLR 7 day | 1.5 | 0.0 |
| PBMC rest | 0.0 | 0.0 |
| PBMC PWM | 0.0 | 0.0 |
| PBMC PHA-L | 0.0 | 0.0 |
| Ramos (B cell) none | 0.0 | 0.0 |
| Ramos (B cell) ionomycin | 0.0 | 0.0 |
| B lymphocytes PWM | 0.0 | 0.0 |
| B lymphocytes CD40L and IL-4 | 0.0 | 0.0 |
| EOL-1 dbcAMP | 0.0 | 0.0 |
| EOL-1 dbcAMP PMA/ionomycin | 0.0 | 0.0 |
| Dendritic cells none | 0.0 | 0.0 |
| Dendritic cells LPS | 0.0 | 0.0 |
| Dendritic cells anti-CD40 | 0.0 | 0.0 |
| Monocytes rest | 0.0 | 0.0 |
| Monocytes LPS | 1.7 | 0.0 |
| Macrophages rest | 0.0 | 0.0 |
| Macrophages LPS | 0.0 | 0.0 |
| HUVEC none | 0.0 | 0.0 |
| HUVEC starved | 0.0 | 0.0 |
| HUVEC IL-1beta | 0.0 | 0.0 |
| HUVEC IFN gamma | 0.4 | 0.0 |
| HUVEC TNF alpha + IFN gamma | 0.0 | 0.0 |
| HUVEC TNF alpha + IL4 | 2.0 | 0.0 |
| HUVEC IL-11 | 0.0 | 0.0 |
| Lung Microvascular EC none | 0.0 | 0.0 |
| Lung Microvascular EC TNFalpha + | 0.0 | 0.0 |
| IL-1beta | | |
| Microvascular Dermal EC none | 0.0 | 0.0 |
| Microsvasular Dermal EC TNFalpha + | 0.0 | 0.0 |
| IL-1beta | | |
| Bronchial epithelium TNFalpha + IL1beta | 0.0 | 0.0 |
| Small airway epithelium none | 0.0 | 0.0 |
| Small airway epithelium TNFalpha + | 6.5 | 11.7 |
| IL-1beta | | |
| Coronery artery SMC rest | 0.7 | 0.0 |
| Coronery artery SMC TNFalpha + IL-1beta | 0.0 | 0.0 |
| Astrocytes rest | 13.9 | 16.8 |
| Astrocytes TNFalpha + IL-1beta | 3.6 | 5.3 |
| KU-812 (Basophil) rest | 0.9 | 1.0 |
| KU-812 (Basophil) PMA/ionomycin | 100.0 | 100.0 |
| CCD1106 (Keratinocytes) none | 0.0 | 0.0 |
| CCD1106 (Keratinocytes) TNFalpha + | 0.0 | 0.0 |
| IL-1beta | | |
| Liver cirrhosis | 1.9 | 3.8 |
| Lupus kidney | 1.3 | 1.4 |
| NCI-H292 none | 42.9 | 48.0 |
| NCI-H292 IL-4 | 14.7 | 14.8 |
| NCI-H292 IL-9 | 40.6 | 36.6 |
| NCI-H292 IL-13 | 12.2 | 7.4 |
| NCI-H292 IFN gamma | 16.8 | 10.9 |
| HPAEC none | 0.0 | 0.0 |
| HPAEC TNF alpha + IL-1 beta | 0.0 | 0.0 |
| Lung fibroblast none | 0.0 | 1.7 |
| Lung fibroblast TNF alpha + IL-1 beta | 0.0 | 0.0 |
| Lung fibroblast IL-4 | 1.7 | 0.0 |
| Lung fibroblast IL-9 | 0.0 | 0.0 |
| Lung fibroblast IL-13 | 0.7 | 0.0 |
| Lung fibroblast IFN gamma | 0.0 | 0.0 |
| Dermal fibroblast CCD1070 rest | 0.0 | 0.0 |
| Dermal fibroblast CCD1070 TNF alpha | 0.0 | 0.0 |
| Dermal fibroblast CCD1070 IL-1 beta | 0.9 | 0.0 |
| Dermal fibroblast IFN gamma | 0.0 | 0.0 |
| Dermal fibroblast IL-4 | 0.0 | 0.0 |
| IBD Colitis 2 | 0.8 | 2.6 |
| IBD Crohn's | 0.0 | 2.0 |
| Colon | 28.1 | 23.2 |
| Lung | 34.9 | 51.4 |
| Thymus | 35.1 | 28.3 |
| Kidney | 8.2 | 7.4 |

Column A - Rel. Exp. (%) Ag1096, Run 160353275
Column B - Pel. Exp. (%) Ag3242, Run 164390548

AI_comprehensive panel_v1.0 Summary: Ag3242 Expression of this gene was ubiquitous in this panel, with high expression in samples derived from patients suffering from ulcerative colitis, Crohns disease and psoriasis (CTs=33). Significant expression was also seen in samples derived from synovium, cartilage and bone of rheumatoid arthritis. Antibody or small molecule therapies designed with the protein encoded for by this gene are useful in the treatment of inflammatory bowel diseases and rheumatoid arthritis.

Panel 1.3D Summary: Ag3242 Highest expression of the CG57448-01 gene was seen in a renal cancer cell line (CT=31.1). Significant expression was also seen in cell lines derived from ovarian cancer, lung cancer, brain cancer and melanoma. Expression of this gene is useful as a marker to differentiate between these samples and other samples on this panel and as a diagnostic marker for the presence of these cancers. This gene encodes a protein that is homologous to cadherin, a cell-adhesion molecule. Therapeutic modulation of the expression or function of this gene or gene prduct is effective in the treatment of lung, renal and melanoma cancers.

Expression of the this gene was also high in many regions of the brain, including the amygdala, thalamus, cerebellum, and cerebral cortex, with highest expression in the hippocampus. Expression was also detected in the spinal cord. Cadherins can act as axon guidance and cell adhesion proteins, specifically during development and in the response to injury (Ranscht B. (2000) Int. J. Dev. Neurosci. 18: 643-651.). Manipulation of levels of this protein are of use in inducing a compensatory synaptogenic response to neuronal death in Alzheimer's disease, Parkinson's disease, Huntington's disease, spinocerebellar ataxia, progressive supranuclear palsy, ALS, head trauma, stroke, or any other disease/condition associated with neuronal loss.

Among tissues with metabolic function, this gene was moderately expressed in pituitary gland, adrenal gland, thyroid, pancreas, skeletal muscle, and liver, reflecting the widespread role of cadherins in cell-cell adhesion. This expression shows that this gene product plays a role in normal metabolic and neuroendocrine function and that dysregulated expression of this gene contributes to metabolic diseases (such as obesity and diabetes) or neuroendocrine disorders.

Panel 2.2 Summary: Ag3242 Highest expression of this gene was seen in a sample derived from an ocular melanoma metastasis (CT=29). Thus, expression of this gene is useful as a marker to differentiate between this sample and other samples on this panel.

Panel 4D Summary: Ag1096/Ag3242 Highest expression of this gene was detected in the basophil cell line (KU-812) treated with PMA/ionomycin (CTs=30-32).

Significant expression was also seen in a cluster of treated and untreated samples derived from the muco-epidermoid cell line NCI-H292. Thus, this gene, which encodes a cadherin homolog, was expressed in both a cell line that is often used as a model of airway epithelium (NCI-H292) and a cell line that is a reasonable model for the inflammatory cells that contribute to various inflammatory lung diseases. Therapeutic modulation of this gene or gene prodcut may is useful to reduce or eliminate the symptoms of patients suffering from pathological and inflammatory lung disorders, including chronic obstructive pulmonary disease, asthma, allergy and emphysema.

Low but significant levels of expression were also seen in the samples derived from normal colon, kidney, lung and thymus. Therapeutic modulation of the expression or function of this gene or gene product is important for maintaining or restoring normal function to this organs during inflammation.

AZ. CG58567-01: Protocadherin

Expression of gene CG58567-01 was assessed using the primer-probe set Ag2897, described in Table AZA. Results of the RTQ-PCR runs are shown in Tables AZB, AZC, AZD, AZE and AZF.

TABLE AZA
Probe Name Ag2897

| | | | | SEQ |
|---|---|---|---|---|
| | | | Start | ID |
| Primers | Sequences | Length | Position | No |
| Forward | 5′-tgacaggggatattcatgctaa-3′ | 22 | 1565 | `1540 |
| Probe | TET-5′-tggcaataaatactgcctcacagtcca-3′- | 27 | 1608 | 1541 |
| | TAMRA | | | |
| Reverse | 5′-agttgcatcacctttgtctttg-3′ | 22 | 1638 | 1542 |

TABLE AZB
AI_comprehensive panel_v1.0

| Tissue Name | A |
|---|---|
| 110967 COPD-F | 4.7 |
| 110980 COPD-F | 22.5 |
| 110968 COPD-M | 8.5 |
| 110977 COPD-M | 51.8 |
| 110989 Emphysema-F | 51.8 |
| 110992 Emphysema-F | 5.0 |
| 110993 Emphysema-F | 5.6 |
| 110994 Emphysema-F | 3.3 |
| 110995 Emphysema-F | 13.2 |
| 110996 Emphysema-F | 0.6 |
| 110997 Asthma-M | 1.4 |
| 111001 Asthma-F | 8.1 |
| 111002 Asthma-F | 12.5 |
| 111003 Atopic Asthma-F | 12.8 |
| 111004 Atopic Asthma-F | 12.3 |
| 111005 Atopic Asthma-F | 6.1 |
| 111006 Atopic Asthma-F | 3.7 |
| 111417 Allergy-M | 7.7 |
| 112347 Allergy-M | 0.8 |
| 112349 Normal Lung-F | 0.8 |
| 112357 Normal Lung-F | 20.2 |
| 112354 Normal Lung-M | 2.1 |
| 112374 Crohns-F | 4.7 |
| 112389 Match Control Crohns-F | 2.1 |
| 112375 Crohns-F | 6.7 |
| 112732 Match Control Crohns-F | 2.2 |
| 112725 Crohns-M | 18.9 |
| 112387 Match Control Crohns-M | 11.2 |
| 112378 Crohns-M | 1.3 |
| 112390 Match Control Crohns-M | 62.4 |
| 112726 Crohns-M | 9.6 |
| 112731 Match Control Crohns-M | 16.0 |
| 112380 Ulcer Col-F | 21.2 |
| 112734 Match Control Ulcer Col-F | 4.5 |
| 112384 Ulcer Col-F | 17.3 |
| 112737 Match Control Ulcer Col-F | 5.2 |
| 112386 Ulcer Col-F | 5.3 |
| 112738 Match Control Ulcer Col-F | 2.7 |
| 112381 Ulcer Col-M | 0.3 |
| 112735 Match Control Ulcer Col-M | 23.7 |
| 112382 Ulcer Col-M | 5.8 |
| 112394 Match Control Ulcer Col-M | 6.5 |
| 112383 Ulcer Col-M | 1.6 |
| 112736 Match Control Ulcer Col-M | 1.3 |
| 112423 Psoriasis-F | 7.9 |
| 112427 Match Control Psoriasis-F | 100.0 |
| 112418 Psoriasis-M | 2.8 |
| 112723 Match Control Psoriasis-M | 14.3 |
| 112419 Psoriasis-M | 10.7 |
| 112424 Match Control Psoriasis-M | 8.7 |
| 112420 Psoriasis-M | 40.1 |
| 112425 Match Control Psoriasis-M | 65.1 |
| 104689 (MF) OA Bone-Backus | 1.7 |
| 104690 (MF) Adj “Normal” Bone-Backus | 4.5 |
| 104691 (MF) OA Synovium-Backus | 9.5 |
| 104692 (BA) OA Cartilage-Backus | 3.1 |
| 104694 (BA) OA Bone-Backus | 1.3 |
| 104695 (BA) Adj “Normal” Bone-Backus | 1.8 |
| 104696 (BA) OA Synovium-Backus | 5.0 |
| 104700 (SS) OA Bone-Backus | 5.2 |
| 104701 (SS) Adj “Normal” Bone-Backus | 7.0 |
| 104702 (SS) OA Synovium-Backus | 12.7 |
| 117093 OA Cartilage Rep7 | 15.1 |
| 112672 OA Bone5 | 15.9 |
| 112673 OA Synovium5 | 8.2 |
| 112674 OA Synovia Fluid cells5 | 7.9 |
| 117100 OA Cartilage Rep14 | 1.3 |
| 112756 OA Bone9 | 0.8 |
| 112757 OA Synovium9 | 0.4 |
| 112758 OA Synovial Fluid Cells9 | 10.2 |
| 117125 RA Cartilage Rep2 | 6.5 |
| 113492 Bone2 RA | 2.2 |
| 113493 Synovium2 RA | 0.2 |
| 113494 Syn Fluid Cells RA | 2.0 |
| 113499 Cartilage4 RA | 1.6 |
| 113500 Bone4 RA | 1.3 |
| 113501 Synovium4 RA | 1.9 |
| 113502 Syn Fluid Cells4 RA | 1.0 |
| 113495 Cartilage3 RA | 1.1 |
| 113496 Bone3 RA | 1.4 |
| 113497 Synovium3 RA | 0.2 |
| 113498 Syn Fluid Cells3 RA | 1.2 |
| 117106 Normal Cartilage Rep20 | 0.3 |
| 113663 Bone3 Normal | 2.8 |
| 113664 Synovium3 Normal | 0.2 |
| 113665 Syn Fluid Cells3 Normal | 1.3 |
| 117107 Normal Cartilage Rep22 | 9.2 |
| 113667 Bone4 Normal | 9.5 |
| 113668 Synovium4 Normal | 6.1 |
| 113669 Syn Fluid Cells4 Normal | 10.2 |

Column A - Rel. Exp. (%) Ag2897, Run 229662505

TABLE AZC
Panel 1.3D

| Tissue Name | A | B |
|---|---|---|
| Liver adenocarcinoma | 0.0 | 0.0 |
| Pancreas | 1.4 | 2.4 |
| Pancreatic ca. CAPAN 2 | 0.0 | 0.0 |
| Adrenal gland | 1.4 | 0.0 |
| Thyroid | 0.0 | 0.0 |
| Salivary gland | 0.0 | 0.0 |
| Pituitary gland | 4.1 | 9.0 |
| Brain (fetal) | 7.1 | 17.8 |
| Brain (whole) | 21.0 | 59.0 |
| Brain (amygdala) | 24.7 | 37.9 |
| Brain (cerebellum) | 5.6 | 28.5 |
| Brain (hippocampus) | 25.2 | 25.3 |
| Brain (substantia nigra) | 0.9 | 17.1 |
| Brain (thalamus) | 4.1 | 23.0 |
| Cerebral Cortex | 100.0 | 22.2 |
| Spinal cord | 68.8 | 100.0 |
| glio/astro U87-MG | 54.3 | 10.4 |
| glio/astro U-118-MG | 1.5 | 20.4 |
| astrocytoma SW 1783 | 16.3 | 0.0 |
| neuro*; met SK-N-AS | 0.0 | 0.0 |
| astrocytoma SF-539 | 0.0 | 4.3 |
| astrocytoma SNB-75 | 18.4 | 28.7 |
| glioma SNB-19 | 0.0 | 2.6 |
| glioma U251 | 3.1 | 27.0 |
| glioma SF-295 | 4.8 | 2.2 |
| Heart (fetal) | 0.0 | 0.0 |
| Heart | 1.4 | 0.0 |
| Skeletal muscle (fetal) | 77.4 | 0.0 |
| Skeletal muscle | 1.1 | 14.1 |
| Bone marrow | 0.0 | 2.5 |
| Thymus | 2.1 | 2.4 |
| Spleen | 0.0 | 4.4 |
| Lymph node | 0.0 | 4.8 |
| Colorectal | 10.7 | 0.0 |
| Stomach | 8.7 | 64.6 |
| Small intestine | 7.2 | 18.9 |
| Colon ca. SW480 | 0.0 | 0.0 |
| Colon ca.* SW620(SW480 met) | 0.0 | 0.0 |
| Colon ca. HT29 | 0.0 | 0.0 |
| Colon ca. HCT-116 | 0.0 | 0.0 |
| Colon ca. CaCo-2 | 0.0 | 0.0 |
| Colon ca. tissue(ODO3866) | 1.7 | 8.1 |
| Colon ca. HCC-2998 | 0.0 | 0.0 |
| Gastric ca.* (liver met) NCI-N87 | 10.3 | 7.7 |
| Bladder | 14.2 | 29.3 |
| Trachea | 4.1 | 0.0 |
| Kidney | 0.0 | 0.0 |
| Kidney (fetal) | 2.8 | 2.4 |
| Renal ca. 786-0 | 0.9 | 3.3 |
| Renal ca. A498 | 13.9 | 23.7 |
| Renal ca. RXF 393 | 0.0 | 0.0 |
| Renal ca. ACHN | 0.0 | 0.0 |
| Renal ca. UO-31 | 0.0 | 0.0 |
| Renal ca. TK-10 | 0.0 | 0.0 |
| Liver | 0.0 | 2.9 |
| Liver (fetal) | 0.4 | 0.0 |
| Liver ca. (hepatoblast) HepG2 | 0.0 | 0.0 |
| Lung | 1.4 | 0.0 |
| Lung (fetal) | 6.8 | 11.0 |
| Lung ca. (small cell) LX-1 | 0.0 | 0.0 |
| Lung ca. (small cell) NCI-H69 | 0.0 | 4.1 |
| Lung ca. (s. cell var.) SHP-77 | 1.0 | 0.0 |
| Lung ca. (large cell)NCI-H460 | 0.0 | 0.0 |
| Lung ca. (non-sm. cell) A549 | 0.0 | 0.0 |
| Lung ca. (non-s. cell) NCI-H23 | 0.0 | 3.0 |
| Lung ca. (non-s. cell) HOP-62 | 0.6 | 7.5 |
| Lung ca. (non-s. cl) NCI-H522 | 0.0 | 0.0 |
| Lung ca. (squam.) SW 900 | 4.6 | 8.6 |
| Lung ca. (squam.) NCI-H596 | 0.0 | 0.0 |
| Mammary gland | 7.9 | 17.1 |
| Breast ca.* (pl. ef) MCF-7 | 0.0 | 0.0 |
| Breast ca.* (pl. ef) MDA-MB-231 | 0.0 | 14.5 |
| Breast ca.* (pl. ef) T47D | 0.0 | 0.0 |
| Breast ca. BT-549 | 0.0 | 12.2 |
| Breast ca. MDA-N | 0.0 | 0.0 |
| Ovary | 12.1 | 6.1 |
| Ovarian ca. OVCAR-3 | 3.8 | 12.1 |
| Ovarian ca. OVCAR-4 | 0.0 | 0.0 |
| Ovarian ca. OVCAR-5 | 32.5 | 25.5 |
| Ovarian ca. OVCAR-8 | 1.9 | 0.0 |
| Ovarian ca. IGROV-1 | 0.0 | 0.0 |
| Ovarian ca.* (ascites) SK-OV-3 | 0.0 | 0.0 |
| Uterus | 12.4 | 66.4 |
| Placenta | 0.0 | 2.3 |
| Prostate | 0.0 | 0.0 |
| Prostate ca.* (bone met)PC-3 | 0.0 | 0.0 |
| Testis | 56.3 | 68.8 |
| Melanoma Hs688(A).T | 1.3 | 8.2 |
| Melanoma* (met) Hs688(B).T | 2.5 | 4.8 |
| Melanoma UACC-62 | 1.5 | 2.8 |
| Melanoma M14 | 0.0 | 0.0 |
| Melanoma LOX IMVI | 0.0 | 0.0 |
| Melanoma* (met) SK-MEL-5 | 0.0 | 0.0 |
| Adipose | 7.2 | 0.0 |

Column A - Rel. Exp. (%) Ag2897, Run 161905860
Column B - Rel. Exp. (%) Ag2897, Run 165518166

TABLE AZD
Panel 2D

| Tissue Name | A | B |
|---|---|---|
| Normal Colon | 83.5 | 90.1 |
| CC Well to Mod Diff (ODO3866) | 0.0 | 7.2 |
| CC Margin (ODO3866) | 16.0 | 16.6 |
| CC Gr.2 rectosigmoid (ODO3868) | 0.0 | 0.0 |
| CC Margin (ODO3868) | 6.3 | 3.6 |
| CC Mod Diff (ODO3920) | 0.9 | 2.1 |
| CC Margin (ODO3920) | 19.1 | 11.7 |
| CC Gr.2 ascend colon (ODO3921) | 8.8 | 9.2 |
| CC Margin (ODO3921) | 6.9 | 7.5 |
| CC from Partial Hepatectomy | 0.9 | 3.3 |
| (ODO4309) Mets | | |
| Liver Margin (ODO4309) | 0.7 | 2.0 |
| Colon mets to lung (OD04451-01) | 0.0 | 0.0 |
| Lung Margin (OD04451-02) | 1.9 | 1.0 |
| Normal Prostate 6546-1 | 3.9 | 10.7 |
| Prostate Cancer (OD04410) | 17.3 | 11.6 |
| Prostate Margin (OD04410) | 25.0 | 15.5 |
| Prostate Cancer (OD04720-01) | 28.3 | 11.7 |
| Prostate Margin (OD04720-02) | 29.9 | 20.4 |
| Normal Lung 061010 | 16.2 | 15.1 |
| Lung Met to Muscle (ODO4286) | 1.2 | 0.0 |
| Muscle Margin (ODO4286) | 8.2 | 1.6 |
| Lung Malignant Cancer (OD03126) | 7.0 | 2.2 |
| Lung Margin (OD03126) | 2.3 | 3.0 |
| Lung Cancer (OD04404) | 8.2 | 3.1 |
| Lung Margin (OD04404) | 11.1 | 10.2 |
| Lung Cancer (OD04565) | 0.0 | 0.9 |
| Lung Margin (OD04565) | 1.9 | 5.0 |
| Lung Cancer (OD04237-01) | 95.3 | 55.9 |
| Lung Margin (OD04237-02) | 4.0 | 2.3 |
| Ocular Mel Met to Liver (ODO4310) | 0.0 | 0.0 |
| Liver Margin (ODO4310) | 2.7 | 0.8 |
| Melanoma Mets to Lung (OD04321) | 0.0 | 1.7 |
| Lung Margin (OD04321) | 10.1 | 6.6 |
| Normal Kidney | 14.8 | 16.4 |
| Kidney Ca, Nuclear grade 2 (OD04338) | 93.3 | 100.0 |
| Kidney Margin (OD04338) | 14.3 | 2.0 |
| Kidney Ca Nuclear grade 1/2 (OD04339) | 5.0 | 2.0 |
| Kidney Margin (OD04339) | 4.1 | 3.0 |
| Kidney Ca, Clear cell type (OD04340) | 27.0 | 17.4 |
| Kidney Margin (OD04340) | 6.4 | 12.7 |
| Kidney Ca, Nuclear grade 3 (OD04348) | 1.9 | 2.1 |
| Kidney Margin (OD04348) | 4.7 | 6.2 |
| Kidney Cancer (OD04622-01) | 50.0 | 38.4 |
| Kidney Margin (OD04622-03) | 4.2 | 0.0 |
| Kidney Cancer (OD04450-01) | 2.6 | 3.6 |
| Kidney Margin (OD04450-03) | 14.2 | 10.5 |
| Kidney Cancer 8120607 | 1.9 | 0.0 |
| Kidney Margin 8120608 | 1.3 | 0.9 |
| Kidney Cancer 8120613 | 1.4 | 1.6 |
| Kidney Margin 8120614 | 0.9 | 1.5 |
| Kidney Cancer 9010320 | 21.6 | 16.0 |
| Kidney Margin 9010321 | 1.9 | 0.5 |
| Normal Uterus | 9.5 | 10.9 |
| Uterus Cancer 064011 | 68.3 | 71.2 |
| Normal Thyroid | 3.3 | 0.8 |
| Thyroid Cancer 064010 | 2.0 | 6.9 |
| Thyroid Cancer A302152 | 8.9 | 12.2 |
| Thyroid Margin A302153 | 1.0 | 0.7 |
| Normal Breast | 15.9 | 15.3 |
| Breast Cancer (OD04566) | 4.3 | 6.3 |
| Breast Cancer (OD04590-01) | 7.3 | 2.8 |
| Breast Cancer Mets (OD04590-03) | 9.4 | 4.8 |
| Breast Cancer Metastasis (OD04655-05) | 3.6 | 0.9 |
| Breast Cancer 064006 | 8.4 | 11.6 |
| Breast Cancer 1024 | 2.6 | 4.3 |
| Breast Cancer 9100266 | 1.9 | 2.0 |
| Breast Margin 9100265 | 7.4 | 5.4 |
| Breast Cancer A209073 | 20.0 | 13.6 |
| Breast Margin A209073 | 5.0 | 12.0 |
| Normal Liver | 0.9 | 1.0 |
| Liver Cancer 064003 | 0.0 | 0.0 |
| Liver Cancer 1025 | 2.4 | 0.0 |
| Liver Cancer 1026 | 0.0 | 0.0 |
| Liver Cancer 6004-T | 0.0 | 1.0 |
| Liver Tissue 6004-N | 0.0 | 0.0 |
| Liver Cancer 6005-T | 0.0 | 0.0 |
| Liver Tissue 6005-N | 0.0 | 0.0 |
| Normal Bladder | 18.0 | 13.8 |
| Bladder Cancer 1023 | 1.0 | 0.7 |
| Bladder Cancer A302173 | 10.9 | 2.6 |
| Bladder Cancer (OD04718-01) | 0.0 | 1.9 |
| Bladder Normal Adjacent (OD04718-03) | 33.2 | 31.0 |
| Normal Ovary | 0.0 | 4.8 |
| Ovarian Cancer 064008 | 100.0 | 62.0 |
| Ovarian Cancer (OD04768-07) | 49.7 | 38.7 |
| Ovary Margin (OD04768-08) | 6.3 | 2.5 |
| Normal Stomach | 28.1 | 18.7 |
| Gastric Cancer 9060358 | 11.0 | 14.1 |
| Stomach Margin 9060359 | 15.9 | 29.7 |
| Gastric Cancer 9060395 | 15.0 | 6.8 |
| Stomach Margin 9060394 | 26.6 | 26.4 |
| Gastric Cancer 9060397 | 2.7 | 1.7 |
| Stomach Margin 9060396 | 28.1 | 33.0 |
| Gastric Cancer 064005 | 27.7 | 33.4 |

Column A - Pel. Exp. (%) Ag2897, Run 161905861
Column B - Rel. Exp. (%) Ag2897, Run 164997312

TABLE AZE
Panel 3D

| Tissue Name | A |
|---|---|
| Daoy- Medulloblastoma | 0.0 |
| TE671- Medulloblastoma | 0.0 |
| D283 Med- Medulloblastoma | 0.0 |
| PFSK-1- Primitive Neuroectodermal | 0.0 |
| XF-498- CNS | 0.6 |
| SNB-78- Glioma | 6.0 |
| SF-268- Glioblastoma | 0.0 |
| T98G- Glioblastoma | 0.0 |
| SK-N-SH- Neuroblastoma (metastasis) | 0.0 |
| SF-295- Glioblastoma | 0.0 |
| Cerebellum | 7.1 |
| Cerebellum | 0.0 |
| NCI-H292- Mucoepidermoid lung carcinoma | 0.0 |
| DMS-114- Small cell lung cancer | 0.0 |
| DMS-79- Small cell lung cancer | 0.8 |
| NCI-H146- Small cell lung cancer | 1.3 |
| NCI-H526- Small cell lung cancer | 100.0 |
| NCI-N417- Small cell lung cancer | 0.0 |
| NCI-H82- Small cell lung cancer | 0.0 |
| NCI-H157- Squamous cell lung cancer (metastasis) | 0.0 |
| NCI-H1155- Large cell lung cancer | 3.2 |
| NCI-H1299- Large cell lung cancer | 1.9 |
| NCI-H727- Lung carcinoid | 0.0 |
| NCI-UMC-11- Lung carcinoid | 2.1 |
| LX-1- Small cell lung cancer | 0.0 |
| Colo-205- Colon cancer | 0.0 |
| KM12- Colon cancer | 2.6 |
| KM20L2- Colon cancer | 0.0 |
| NCI-H716- Colon cancer | 31.0 |
| SW-48- Colon adenocarcinoma | 0.0 |
| SW1116- Colon adenocarcinoma | 0.0 |
| LS 174T- Colon adenocarcinoma | 0.0 |
| SW-948- Colon adenocarcinoma | 0.0 |
| SW-480- Colon adenocarcinoma | 1.5 |
| NCI-SNU-5- Gastric carcinoma | 0.0 |
| KATO III- Gastric carcinoma | 0.0 |
| NCI-SNU-16- Gastric carcinoma | 0.8 |
| NCI-SNU-1- Gastric carcinoma | 0.0 |
| RF-1- Gastric adenocarcinoma | 0.0 |
| RF-48- Gastric adenocarcinoma | 0.8 |
| MKN-45- Gastric carcinoma | 0.0 |
| NCI-N87- Gastric carcinoma | 0.3 |
| OVCAR-5- Ovarian carcinoma | 0.0 |
| RL95-2- Uterine carcinoma | 0.0 |
| HelaS3- Cervical adenocarcinoma | 0.0 |
| Ca Ski- Cervical epidermoid carcinoma (metastasis) | 0.0 |
| ES-2- Ovarian clear cell carcinoma | 0.8 |
| Ramos- Stimulated with PMA/ionomycin 6 h | 0.0 |
| Ramos- Stimulated with PMA/ionomycin 14 h | 0.0 |
| MEG-01- Chronic myelogenous leukemia | 0.0 |
| (megokaryoblast) | |
| Raji- Burkitt's lymphoma | 0.0 |
| Daudi- Burkitt's lymphoma | 0.0 |
| U266- B-cell plasmacytoma | 0.0 |
| CA46- Burkitt's lymphoma | 0.0 |
| RL- non-Hodgkin's B-cell lymphoma | 0.0 |
| JM1- pre-B-cell lymphoma | 0.0 |
| Jurkat- T cell leukemia | 0.0 |
| TF-1- Erythroleukemia | 0.0 |
| HUT 78- T-cell lymphoma | 0.0 |
| U937- Histiocytic lymphoma | 0.0 |
| KU-812- Myelogenous leukemia | 0.0 |
| 769-P- Clear cell renal carcinoma | 0.0 |
| Caki-2- Clear cell renal carcinoma | 0.9 |
| SW 839- Clear cell renal carcinoma | 0.0 |
| Rhabdoid kidney tumor | 0.0 |
| Hs766T- Pancreatic carcinoma (LN metastasis) | 0.0 |
| CAPAN-1- Pancreatic adenocarcinoma (liver | 0.0 |
| metastasis) | |
| SU86.86- Pancreatic carcinoma (liver metastasis) | 0.6 |
| BxPC-3- Pancreatic adenocarcinoma | 0.0 |
| HPAC- Pancreatic adenocarcinoma | 0.0 |
| MIA PaCa-2- Pancreatic carcinoma | 0.0 |
| CFPAC-1- Pancreatic ductal adenocarcinoma | 0.4 |
| PANC-1- Pancreatic epithelioid ductal carcinoma | 0.0 |
| T24- Bladder carcinma (transitional cell) | 0.0 |
| 5637- Bladder carcinoma | 0.0 |
| HT-1197- Bladder carcinoma | 0.0 |
| UM-UC-3- Bladder carcinma (transitional cell) | 0.0 |
| A204- Rhabdomyosarcoma | 0.0 |
| HT-1080- Fibrosarcoma | 0.8 |
| MG-63- Osteosarcoma | 0.0 |
| SK-LMS-1- Leiomyosarcoma (vulva) | 0.0 |
| SJRH30- Rhabdomyosarcoma (met to bone marrow) | 0.0 |
| A431- Epidermoid carcinoma | 0.0 |
| WM266-4- Melanoma | 1.2 |
| DU 145- Prostate carcinoma (brain metastasis) | 0.0 |
| MDA-MB-468- Breast adenocarcinoma | 0.8 |
| SCC-4- Squamous cell carcinoma of tongue | 0.0 |
| SCC-9- Squamous cell carcinoma of tongue | 0.0 |
| SCC-15- Squamous cell carcinoma of tongue | 0.0 |
| CAL 27- Squamous cell carcinoma of tongue | 0.0 |

Column A - Rel. Exp. (%) Ag2897, Run 164629841

TABLE AZF
Panel 4D

| Tissue Name | A | B |
|---|---|---|
| Secondary Th1 act | 0.0 | 0.0 |
| Secondary Th2 act | 0.0 | 0.0 |
| Secondary Tr1 act | 0.0 | 0.0 |
| Secondary Th1 rest | 0.0 | 0.0 |
| Secondary Th2 rest | 0.0 | 0.0 |
| Secondary Tr1 rest | 0.0 | 0.0 |
| Primary Th1 act | 0.0 | 0.0 |
| Primary Th2 act | 0.0 | 0.0 |
| Primary Tr1 act | 0.0 | 0.0 |
| Primary Th1 rest | 9.3 | 20.7 |
| Primary Th2 rest | 0.0 | 9.0 |
| Primary Tr1 rest | 9.8 | 0.0 |
| CD45RA CD4 lymphocyte act | 0.0 | 0.0 |
| CD45RO CD4 lymphocyte act | 0.0 | 0.0 |
| CD8 lymphocyte act | 0.0 | 0.0 |
| Secondary CD8 lymphocyte rest | 0.0 | 0.0 |
| Secondary CD8 lymphocyte act | 0.0 | 0.0 |
| CD4 lymphocyte none | 0.0 | 0.0 |
| 2ry Th1/Th2/Tr1_anti-CD95 CH11 | 0.0 | 0.0 |
| LAK cells rest | 10.9 | 0.0 |
| LAK cells IL-2 | 0.0 | 0.0 |
| LAK cells IL-2 + IL-12 | 0.0 | 0.0 |
| LAK cells IL-2 + IFN gamma | 0.0 | 0.0 |
| LAK cells IL-2 + IL-18 | 0.0 | 0.0 |
| LAK cells PMA/ionomycin | 0.0 | 0.0 |
| NK Cells IL-2 rest | 0.0 | 0.0 |
| Two Way MLR 3 day | 0.0 | 9.3 |
| Two Way MLR 5 day | 0.0 | 0.0 |
| Two Way MLR 7 day | 0.0 | 0.0 |
| PBMC rest | 0.0 | 0.0 |
| PBMC PWM | 0.0 | 0.0 |
| PBMC PHA-L | 8.9 | 0.0 |
| Ramos (B cell) none | 0.0 | 0.0 |
| Ramos (B cell) ionomycin | 0.0 | 0.0 |
| B lymphocytes PWM | 0.0 | 0.0 |
| B lymphocytes CD40L and IL-4 | 0.0 | 0.0 |
| EOL-1 dbcAMP | 0.0 | 0.0 |
| EOL-1 dbcAMP PMA/ionomycin | 0.0 | 0.0 |
| Dendritic cells none | 0.0 | 0.0 |
| Dendritic cells LPS | 0.0 | 0.0 |
| Dendritic cells anti-CD40 | 0.0 | 0.0 |
| Monocytes rest | 0.0 | 0.0 |
| Monocytes LPS | 0.0 | 0.0 |
| Macrophages rest | 0.0 | 0.0 |
| Macrophages LPS | 0.0 | 0.0 |
| HUVEC none | 0.0 | 0.0 |
| HUVEC starved | 0.0 | 0.0 |
| HUVEC IL-1beta | 0.0 | 0.0 |
| HUVEC IFN gamma | 0.0 | 0.0 |
| HUVEC TNF alpha + IFN gamma | 0.0 | 0.0 |
| HUVEC TNF alpha + IL4 | 0.0 | 0.0 |
| HUVEC IL-11 | 0.0 | 0.0 |
| Lung Microvascular EC none | 0.0 | 0.0 |
| Lung Microvascular EC TNFalpha + | 7.9 | 22.8 |
| IL-1beta | | |
| Microvascular Dermal EC none | 0.0 | 0.0 |
| Microsvasular Dermal EC TNFalpha + | 16.2 | 0.0 |
| IL-1beta | | |
| Bronchial epithelium TNFalpha + IL1beta | 4.4 | 0.0 |
| Small airway epithelium none | 10.4 | 9.7 |
| Small airway epithelium TNFalpha + | 100.0 | 75.8 |
| IL-1beta | | |
| Coronery artery SMC rest | 0.0 | 9.9 |
| Coronery artery SMC TNFalpha + IL-1beta | 16.8 | 9.0 |
| Astrocytes rest | 40.6 | 0.0 |
| Astrocytes TNFalpha + IL-1beta | 56.6 | 100.0 |
| KU-812 (Basophil) rest | 0.0 | 0.0 |
| KU-812 (Basophil) PMA/ionomycin | 0.0 | 0.0 |
| CCD1106 (Keratinocytes) none | 0.0 | 0.0 |
| CCD1106 (Keratinocytes) TNFalpha + | 0.0 | 0.0 |
| IL-1beta | | |
| Liver cirrhosis | 17.2 | 8.2 |
| Lupus kidney | 8.4 | 9.6 |
| NCI-H292 none | 0.0 | 0.0 |
| NCI-H292 IL-4 | 0.0 | 0.0 |
| NCI-H292 IL-9 | 0.0 | 0.0 |
| NCI-H292 IL-13 | 0.0 | 0.0 |
| NCI-H292 IFN gamma | 0.0 | 0.0 |
| HPAEC none | 0.0 | 0.0 |
| HPAEC TNF alpha + IL-1 beta | 0.0 | 0.0 |
| Lung fibroblast none | 0.0 | 0.0 |
| Lung fibroblast TNF alpha + IL-1 beta | 0.0 | 18.8 |
| Lung fibroblast IL-4 | 0.0 | 17.7 |
| Lung fibroblast IL-9 | 0.0 | 0.0 |
| Lung fibroblast IL-13 | 0.0 | 0.0 |
| Lung fibroblast IFN gamma | 0.0 | 0.0 |
| Dermal fibroblast CCD1070 rest | 18.7 | 5.1 |
| Dermal fibroblast CCD1070 TNF alpha | 45.4 | 29.5 |
| Dermal fibroblast CCD1070 IL-1 beta | 33.7 | 85.9 |
| Dermal fibroblast IFN gamma | 0.0 | 0.0 |
| Dermal fibroblast IL-4 | 0.0 | 8.4 |
| IBD Colitis 2 | 0.0 | 9.9 |
| IBD Crohn's | 7.7 | 0.0 |
| Colon | 62.4 | 39.2 |
| Lung | 9.9 | 22.1 |
| Thymus | 27.7 | 35.8 |
| Kidney | 18.9 | 20.6 |

Column A - Rel. Exp. (%) Ag2897, Run 161905862
Column B - Rel. Exp. (%) Ag2897, Run 163586314

AI_comprehensive panel_v1.0 Summary: Ag2897 Highest expression of this gene was detected in a normal tissue sample adjacent to a psoriasis sample (CT=30). This expression was down-regulated in the corresponding psoriasis sample (CT=33). Expression of this gene is useful as a marker to distinguish between these samples. The expression of this gene was up-regulated in lung from emphysema and COPD patients, consistent with its expression in “stressed” small airway epithelium, lung fibroblasts and lung endothelium (treated with TNF-a and IL-1). Therapeutic modulation of the expression of this gene or its protein and/or signaling via this protein by antibodies, small moleculesvor protein therapeutics is useful for the inhibition of inflammation in lung tissue due to asthma, emphysema and other COPD type diseases

Panel 1.3D Summary: Ag2897 Highest expression of this gene, a protocadherin homolog, was detected in the spinal cord and the cerebral cortex (CTs=31.5). Low levels of expression were also seen in other regions of the brain including the amygdala and the hippocampus. The cadherins have been shown to be critical for CNS development, specifically for the guidance of axons, dendrites and/or growth cones in general. Therapeutic modulation of the levels of this protein, or possible signaling via this protein are of utility in enhancing/directing compensatory synaptogenesis and fiber growth in the CNS in response to neuronal death (stroke, head trauma), axon lesion (spinal cord injury), or neurodegeneration (Alzheimer's, Parkinson's, Huntington's, vascular dementia or any neurodegenerative disease). Since protocadherins play an important role in synaptogenesis, this gene product is also involved in depression, schizophrenia, both of which involve synaptogeneisis. Because this cadherin shows highest expression in the cerebellum, this is also an excellent candidate for the spinocerebellar ataxias as well.

Significant levels of expression were also seen in cell lines derived from ovarian, renal and brain cancers. Thus, expression of this gene or gene product is useful as a marker to differentiate between these samples and other samples on this panel and as a marker to detect the presence of these cancers. Therapeutic modulation of the expression or function of this gene or gene product is effective in the treatment of ovarian, renal and brain cancers.

Panel 2D Summary: Ag2897 Highest expression of this gene was detected in ovarian and kidney cancers (CTs=30.5). Significant levels of expression were also seen in lung and uterine cancers. Higher levels of expression were seen in these cancers than in the corresponding normal adjacent tissues. Therapeutic targeting of this gene or gene product with a small molecule drug, protein therapeutic, or human monoclonal antibody is useful for limiting or blocking the extent of tumor cell migration and invasion, preferably in kidney, lung, uterine and ovarian tumor tumors.

Panel 3D Summary: Ag2897 Highest expression of this gene was seen in a lung cancer cell line (CT=30. 1). Expression of this gene or gene product is useful as a marker to differentiate between this sample and other samples on this panel.

Panel 4D Summary: Ag2897 This gene highly was up-regulated in small airway epithelium and astrocytes stimulated with TNF-alpha and IL-1 beta (CTs=33-34). Other tissues in the lung also up regulated the expression of this gene including lung microvascular endothelium and lung fibroblasts in response to TNF alpha or the Th2 elaborated cytokine IL-4. This demonstrates that this molecule is expressed as a result of inflammation, particularly during asthma since TNFalpha and IL-4 may play important roles in the pathology of this disease. Based on the expression profile of this transcript and the types of cytokines which induce it, antibodies, protein therapeutics or small molecule drugs that target this gene or gene product are useful for inhibiting inflammation in lung tissue due to asthma, emphysema and other COPD type diseases.

BA. CG59534-03: Ovostatin

Expression of gene CG59534-03 was assessed using the primer-probe set

Ag7218, described in Table BAA. Results of the RTQ-PCR runs are shown in Table BAB.

TABLE BAA
Probe Name Ag7218

| | | | | SEQ |
|---|---|---|---|---|
| | | | Start | ID |
| Primers | Sequences | Length | Position | No |
| Forward | 5′-ctacaggaagtgcagcacaac-3′ | 21 | 294 | 1543 |
| Probe | TET-5′-agccagctgaaggcctacctttcctt-3′- | 26 | 336 | 1544 |
| | TAMRA | | | |
| Reverse | 5′-gttgttcaaactcagatctaacacttct-3′ | 28 | 362 | 1545 |

TABLE BAB
Panel 4.1D

| Tissue Name | A |
|---|---|
| Secondary Th1 act | 31.6 |
| Secondary Th2 act | 27.4 |
| Secondary Tr1 act | 14.4 |
| Secondary Th1 rest | 7.4 |
| Secondary Th2 rest | 6.6 |
| Secondary Tr1 rest | 6.0 |
| Primary Th1 act | 9.0 |
| Primary Th2 act | 40.1 |
| Primary Tr1 act | 39.8 |
| Primary Th1 rest | 3.9 |
| Primary Th2 rest | 1.6 |
| Primary Tr1 rest | 0.0 |
| CD45RA CD4 lymphocyte act | 31.4 |
| CD45RO CD4 lymphocyte act | 39.8 |
| CD8 lymphocyte act | 24.0 |
| Secondary CD8 lymphocyte rest | 90.8 |
| Secondary CD8 lymphocyte act | 9.5 |
| CD4 lymphocyte none | 7.5 |
| 2ry Th1/Th2/Tr1_anti-CD95 CH11 | 11.7 |
| LAK cells rest | 20.0 |
| LAK cells IL-2 | 6.0 |
| LAK cells IL-2 + IL-12 | 3.6 |
| LAK cells IL-2 + IFN gamma | 26.6 |
| LAK cells IL-2 + IL-18 | 10.4 |
| LAK cells PMA/ionomycin | 52.1 |
| NK Cells IL-2 rest | 52.9 |
| Two Way MLR 3 day | 12.2 |
| Two Way MLR 5 day | 9.6 |
| Two Way MLR 7 day | 11.9 |
| PBMC rest | 2.5 |
| PBMC PWM | 21.8 |
| PBMC PHA-L | 18.8 |
| Ramos (B cell) none | 54.7 |
| Ramos (B cell) ionomycin | 79.6 |
| B lymphocytes PWM | 13.9 |
| B lymphocytes CD40L and IL-4 | 16.5 |
| EOL-1 dbcAMP | 0.0 |
| EOL-1 dbcAMP PMA/ionomycin | 1.3 |
| Dendritic cells none | 5.9 |
| Dendritic cells LPS | 0.7 |
| Dendritic cells anti-CD40 | 1.3 |
| Monocytes rest | 0.0 |
| Monocytes LPS | 5.7 |
| Macrophages rest | 1.1 |
| Macrophages LPS | 0.0 |
| HUVEC none | 17.1 |
| HUVEC starved | 45.1 |
| HUVEC IL-1beta | 45.7 |
| HUVEC IFN gamma | 26.8 |
| HUVEC TNF alpha + IFN gamma | 12.0 |
| HUVEC TNF alpha + IL4 | 26.4 |
| HUVEC IL-11 | 27.0 |
| Lung Microvascular EC none | 28.5 |
| Lung Microvascular EC TNFalpha + IL-1beta | 15.5 |
| Microvascular Dermal EC none | 8.2 |
| Microsvasular Dermal EC TNFalpha + IL-1beta | 6.9 |
| Bronchial epithelium TNFalpha + IL1beta | 12.9 |
| Small airway epithelium none | 5.1 |
| Small airway epithelium TNFalpha + IL-1beta | 10.5 |
| Coronery artery SMC rest | 15.8 |
| Coronery artery SMC TNFalpha + IL-1beta | 29.3 |
| Astrocytes rest | 15.2 |
| Astrocytes TNFalpha + IL-1beta | 17.2 |
| KU-812 (Basophil) rest | 5.8 |
| KU-812 (Basophil) PMA/ionomycin | 11.7 |
| CCD1106 (Keratinocytes) none | 15.5 |
| CCD1106 (Keratinocytes) TNFalpha + IL-1beta | 8.0 |
| Liver cirrhosis | 8.0 |
| NCI-H292 none | 2.7 |
| NCI-H292 IL-4 | 2.0 |
| NCI-H292 IL-9 | 8.9 |
| NCI-H292 IL-13 | 2.0 |
| NCI-H292 IFN gamma | 5.3 |
| HPAEC none | 21.5 |
| HPAEC TNF alpha + IL-1 beta | 100.0 |
| Lung fibroblast none | 77.9 |
| Lung fibroblast TNF alpha + IL-1 beta | 93.3 |
| Lung fibroblast IL-4 | 33.4 |
| Lung fibroblast IL-9 | 62.4 |
| Lung fibroblast IL-13 | 40.9 |
| Lung fibroblast IFN gamma | 54.7 |
| Dermal fibroblast CCD1070 rest | 8.0 |
| Dermal fibroblast CCD1070 TNF alpha | 29.3 |
| Dermal fibroblast CCD1070 IL-1 beta | 76.3 |
| Dermal fibroblast IFN gamma | 7.0 |
| Dermal fibroblast IL-4 | 26.1 |
| Dermal Fibroblasts rest | 15.8 |
| Neutrophils TNFa + LPS | 0.0 |
| Neutrophils rest | 2.9 |
| Colon | 3.6 |
| Lung | 6.3 |
| Thymus | 4.7 |
| Kidney | 13.7 |

Column A - Rel. Exp. (%) Ag7218, Run 288211130

Panel 4.1D Summary: Ag7218 Highest expression of this gene was seen in human pulmonary artery endothelial cells (CT=32). Detectable levels of expression were also seen in clusters of lung and dermal fibroblasts as well as in activated T cells. Therefore, targeting this gene or its protein product is useful for the reduction or elimination of the inflammation in lung, skin, and T cell mediated inflammatory and autoimmune diseases.

BB. CG59584-02 and CG59584-03: Ovastatin

Expression of gene CG59584-03 was assessed using the primer-probe set Ag1282, described in Table BBA. Results of the RTQ-PCR runs are shown in Tables BBB, BBC, BBD, BBE, BBF, and BBG.

TABLE BBA
Probe Name Ag1282

| | | | | SEQ |
|---|---|---|---|---|
| | | | Start | ID |
| Primers | Sequences | Length | Position | No |
| Forward | 5′-ttcgcaataaatccagtatggt-3′ | 22 | 4510 | 1546 |
| Probe | TET-5′-tgctatcaggatttactccaaccatgtca-3′- | 29 | 4549 | 1547 |
| | TAMRA | | | |
| Reverse | 5′-ttgttttcaagctcttcaatgg-3′ | 22 | 4579 | 1548 |

TABLE BBB
General_screening_panel_v1.4

| Tissue Name | A |
|---|---|
| Adipose | 0.5 |
| Melanoma* Hs688(A).T | 0.0 |
| Melanoma* Hs688(B).T | 0.0 |
| Melanoma* M14 | 100.0 |
| Melanoma* LOXIMVI | 0.9 |
| Melanoma* SK-MEL-5 | 11.2 |
| Squamous cell carcinoma SCC-4 | 0.0 |
| Testis Pool | 5.3 |
| Prostate ca.* (bone met) PC-3 | 0.0 |
| Prostate Pool | 0.3 |
| Placenta | 0.4 |
| Uterus Pool | 0.2 |
| Ovarian ca. OVCAR-3 | 2.1 |
| Ovarian ca. SK-OV-3 | 0.7 |
| Ovarian ca. OVCAR-4 | 0.2 |
| Ovarian ca. OVCAR-5 | 0.8 |
| Ovarian ca. IGROV-1 | 0.2 |
| Ovarian ca. OVCAR-8 | 0.1 |
| Ovary | 0.6 |
| Breast ca. MCF-7 | 0.3 |
| Breast ca. MDA-MB-231 | 0.6 |
| Breast ca. BT 549 | 0.9 |
| Breast ca. T47D | 0.8 |
| Breast ca. MDA-N | 45.1 |
| Breast Pool | 0.7 |
| Trachea | 0.5 |
| Lung | 0.3 |
| Fetal Lung | 4.9 |
| Lung ca. NCI-N417 | 0.0 |
| Lung ca. LX-1 | 1.6 |
| Lung ca. NCI-H146 | 2.3 |
| Lung ca. SHP-77 | 1.1 |
| Lung ca. A549 | 1.1 |
| Lung ca. NCI-H526 | 0.4 |
| Lung ca. NCI-H23 | 2.8 |
| Lung ca. NCI-H460 | 0.1 |
| Lung ca. HOP-62 | 0.7 |
| Lung ca. NCI-H522 | 2.0 |
| Liver | 0.0 |
| Fetal Liver | 0.9 |
| Liver ca. HepG2 | 0.3 |
| Kidney Pool | 1.0 |
| Fetal Kidney | 3.6 |
| Renal ca. 786-0 | 0.6 |
| Renal ca. A498 | 0.0 |
| Renal ca. ACHN | 0.6 |
| Renal ca. UO-31 | 0.1 |
| Renal ca. TK-10 | 1.1 |
| Bladder | 0.4 |
| Gastric ca. (liver met.) NCI-N87 | 1.7 |
| Gastric ca. KATO III | 1.2 |
| Colon ca. SW-948 | 0.0 |
| Colon ca. SW480 | 3.3 |
| Colon ca.* (SW480 met) SW620 | 1.5 |
| Colon ca. HT29 | 0.4 |
| Colon ca. HCT-116 | 2.5 |
| Colon ca. CaCo-2 | 0.6 |
| Colon cancer tissue | 0.5 |
| Colon ca. SW1116 | 0.0 |
| Colon ca. Colo-205 | 0.1 |
| Colon ca. SW-48 | 0.0 |
| Colon Pool | 0.5 |
| Small Intestine Pool | 1.4 |
| Stomach Pool | 0.2 |
| Bone Marrow Pool | 0.2 |
| Fetal Heart | 1.6 |
| Heart Pool | 0.2 |
| Lymph Node Pool | 0.4 |
| Fetal Skeletal Muscle | 1.6 |
| Skeletal Muscle Pool | 0.1 |
| Spleen Pool | 0.8 |
| Thymus Pool | 2.5 |
| CNS cancer (glio/astro) U87-MG | 0.4 |
| CNS cancer (glio/astro) U-118-MG | 0.3 |
| CNS cancer (neuro; met) SK-N-AS | 0.5 |
| CNS cancer (astro) SF-539 | 0.1 |
| CNS cancer (astro) SNB-75 | 9.0 |
| CNS cancer (glio) SNB-19 | 0.2 |
| CNS cancer (glio) SF-295 | 0.7 |
| Brain (Amygdala) Pool | 0.9 |
| Brain (cerebellum) | 0.6 |
| Brain (fetal) | 2.9 |
| Brain (Hippocampus) Pool | 1.0 |
| Cerebral Cortex Pool | 1.1 |
| Brain (Substantia nigra) Pool | 0.8 |
| Brain (Thalamus) Pool | 1.1 |
| Brain (whole) | 0.8 |
| Spinal Cord Pool | 0.5 |
| Adrenal Gland | 0.1 |
| Pituitary gland Pool | 0.1 |
| Salivary Gland | 0.1 |
| Thyroid (female) | 0.1 |
| Pancreatic ca. CAPAN2 | 1.3 |
| Pancreas Pool | 0.9 |

Column A - Rel. Exp. (%) Ag1282, Run 216588406

TABLE BBC
Panel 1.3D

| Tissue Name | A |
|---|---|
| Liver adenocarcinoma | 9.2 |
| Pancreas | 0.5 |
| Pancreatic ca. CAPAN 2 | 1.3 |
| Adrenal gland | 0.3 |
| Thyroid | 0.3 |
| Salivary gland | 0.1 |
| Pituitary gland | 0.3 |
| Brain (fetal) | 11.1 |
| Brain (whole) | 0.7 |
| Brain (amygdala) | 1.1 |
| Brain (cerebellum) | 0.3 |
| Brain (hippocampus) | 1.5 |
| Brain (substantia nigra) | 2.3 |
| Brain (thalamus) | 2.4 |
| Cerebral Cortex | 1.0 |
| Spinal cord | 0.7 |
| glio/astro U87-MG | 0.4 |
| glio/astro U-118-MG | 0.4 |
| astrocytoma SW1783 | 0.4 |
| neuro*; met SK-N-AS | 0.7 |
| astrocytoma SF-539 | 0.3 |
| astrocytoma SNB-75 | 7.8 |
| glioma SNB-19 | 0.2 |
| glioma U251 | 1.2 |
| glioma SF-295 | 0.6 |
| Heart (fetal) | 5.1 |
| Heart | 0.7 |
| Skeletal muscle (fetal) | 3.3 |
| Skeletal muscle | 0.5 |
| Bone marrow | 3.9 |
| Thymus | 15.9 |
| Spleen | 1.2 |
| Lymph node | 4.6 |
| Colorectal | 2.5 |
| Stomach | 0.6 |
| Small intestine | 1.8 |
| Colon ca. SW480 | 2.8 |
| Colon ca.* SW620(SW480 met) | 5.3 |
| Colon ca. HT29 | 0.3 |
| Colon ca. HCT-116 | 1.6 |
| Colon ca. CaCo-2 | 0.8 |
| Colon ca. tissue(ODO3866) | 0.2 |
| Colon ca. HCC-2998 | 3.1 |
| Gastric ca.* (liver met) NCI-N87 | 1.9 |
| Bladder | 0.9 |
| Trachea | 0.3 |
| Kidney | 0.4 |
| Kidney (fetal) | 4.9 |
| Renal ca. 786-0 | 0.3 |
| Renal ca. A498 | 1.9 |
| Renal ca. RXF 393 | 0.1 |
| Renal ca. ACHN | 0.8 |
| Renal ca. UO-31 | 0.4 |
| Renal ca. TK-10 | 2.1 |
| Liver | 0.2 |
| Liver (fetal) | 1.4 |
| Liver ca. (hepatoblast) HepG2 | 0.7 |
| Lung | 0.5 |
| Lung (fetal) | 8.0 |
| Lung ca. (small cell) LX-1 | 1.3 |
| Lung ca. (small cell) NCI-H69 | 13.2 |
| Lung ca. (s. cell var.) SHP-77 | 5.9 |
| Lung ca. (large cell)NCI-H460 | 0.1 |
| Lung ca. (non-sm. cell) A549 | 0.3 |
| Lung ca. (non-s. cell) NCI-H23 | 2.4 |
| Lung ca. (non-s. cell) HOP-62 | 1.4 |
| Lung ca. (non-s. cl) NCI-H522 | 2.6 |
| Lung ca. (squam.) SW 900 | 2.5 |
| Lung ca. (squam.) NCI-H596 | 49.7 |
| Mammary gland | 2.5 |
| Breast ca.* (pl. ef) MCF-7 | 0.5 |
| Breast ca.* (pl. ef) MDA-MB-231 | 0.8 |
| Breast ca.* (pl. ef) T47D | 0.3 |
| Breast ca. BT-549 | 0.1 |
| Breast ca. MDA-N | 100.0 |
| Ovary | 1.3 |
| Ovarian ca. OVCAR-3 | 2.6 |
| Ovarian ca. OVCAR-4 | 0.1 |
| Ovarian ca. OVCAR-5 | 2.7 |
| Ovarian ca. OVCAR-8 | 0.1 |
| Ovarian ca. IGROV-1 | 0.6 |
| Ovarian ca.* (ascites) SK-OV-3 | 1.3 |
| Uterus | 1.4 |
| Placenta | 0.2 |
| Prostate | 0.5 |
| Prostate ca.* (bone met)PC-3 | 0.0 |
| Testis | 17.4 |
| Melanoma Hs688(A).T | 0.0 |
| Melanoma* (met) Hs688(B).T | 0.0 |
| Melanoma UACC-62 | 2.8 |
| Melanoma M14 | 79.6 |
| Melanoma LOX IMVI | 2.1 |
| Melanoma* (met) SK-MEL-5 | 9.9 |
| Adipose | 1.9 |

Column A - Rel. Exp. (%) Ag1282, Run 167614616

TABLE BBD
Panel 2D

| Tissue Name | A |
|---|---|
| Normal Colon | 3.3 |
| CC Well to Mod Diff (ODO3866) | 0.4 |
| CC Margin (ODO3866) | 0.4 |
| CC Gr.2 rectosigmoid (ODO3868) | 1.3 |
| CC Margin (ODO3868) | 0.1 |
| CC Mod Diff (ODO3920) | 3.6 |
| CC Margin (ODO3920) | 0.8 |
| CC Gr.2 ascend colon (ODO3921) | 0.9 |
| CC Margin (ODO3921) | 0.4 |
| CC from Partial Hepatectomy (ODO4309) Mets | 0.9 |
| Liver Margin (ODO4309) | 0.2 |
| Colon mets to lung (OD04451-01) | 0.2 |
| Lung Margin (OD04451-02) | 0.2 |
| Normal Prostate 6546-1 | 1.8 |
| Prostate Cancer (OD04410) | 0.8 |
| Prostate Margin (OD04410) | 1.1 |
| Prostate Cancer (OD04720-01) | 0.7 |
| Prostate Margin (OD04720-02) | 1.6 |
| Normal Lung 061010 | 2.6 |
| Lung Met to Muscle (ODO4286) | 0.3 |
| Muscle Margin (ODO4286) | 0.2 |
| Lung Malignant Cancer (OD03126) | 0.9 |
| Lung Margin (OD03126) | 0.7 |
| Lung Cancer (OD04404) | 0.5 |
| Lung Margin (OD04404) | 0.6 |
| Lung Cancer (OD04565) | 0.8 |
| Lung Margin (OD04565) | 0.4 |
| Lung Cancer (OD04237-01) | 25.5 |
| Lung Margin (OD04237-02) | 0.7 |
| Ocular Mel Met to Liver (ODO4310) | 100.0 |
| Liver Margin (ODO4310) | 0.4 |
| Melanoma Mets to Lung (OD04321) | 21.8 |
| Lung Margin (OD04321) | 0.5 |
| Normal Kidney | 0.5 |
| Kidney Ca, Nuclear grade 2 (OD04338) | 0.4 |
| Kidney Margin (OD04338) | 0.7 |
| Kidney Ca Nuclear grade 1/2 (OD04339) | 0.2 |
| Kidney Margin (OD04339) | 0.1 |
| Kidney Ca, Clear cell type (OD04340) | 0.8 |
| Kidney Margin (OD04340) | 0.6 |
| Kidney Ca, Nuclear grade 3 (OD04348) | 0.5 |
| Kidney Margin (OD04348) | 0.2 |
| Kidney Cancer (OD04622-01) | 0.3 |
| Kidney Margin (OD04622-03) | 0.0 |
| Kidney Cancer (OD04450-01) | 0.1 |
| Kidney Margin (OD04450-03) | 0.1 |
| Kidney Cancer 8120607 | 0.0 |
| Kidney Margin 8120608 | 0.0 |
| Kidney Cancer 8120613 | 0.1 |
| Kidney Margin 8120614 | 0.1 |
| Kidney Cancer 9010320 | 0.1 |
| Kidney Margin 9010321 | 0.1 |
| Normal Uterus | 0.3 |
| Uterus Cancer 064011 | 2.5 |
| Normal Thyroid | 0.4 |
| Thyroid Cancer 064010 | 0.3 |
| Thyroid Cancer A302152 | 0.2 |
| Thyroid Margin A302153 | 0.4 |
| Normal Breast | 1.8 |
| Breast Cancer (OD04566) | 1.6 |
| Breast Cancer (OD04590-01) | 0.7 |
| Breast Cancer Mets (OD04590-03) | 2.3 |
| Breast Cancer Metastasis (OD04655-05) | 0.7 |
| Breast Cancer 064006 | 0.8 |
| Breast Cancer 1024 | 1.2 |
| Breast Cancer 9100266 | 0.9 |
| Breast Margin 9100265 | 0.5 |
| Breast Cancer A209073 | 1.6 |
| Breast Margin A209073 | 1.5 |
| Normal Liver | 0.1 |
| Liver Cancer 064003 | 0.3 |
| Liver Cancer 1025 | 0.1 |
| Liver Cancer 1026 | 0.1 |
| Liver Cancer 6004-T | 0.0 |
| Liver Tissue 6004-N | 0.9 |
| Liver Cancer 6005-T | 0.3 |
| Liver Tissue 6005-N | 0.0 |
| Normal Bladder | 1.1 |
| Bladder Cancer 1023 | 0.2 |
| Bladder Cancer A302173 | 6.5 |
| Bladder Cancer (OD04718-01) | 1.4 |
| Bladder Normal Adjacent (OD04718-03) | 0.7 |
| Normal Ovary | 0.8 |
| Ovarian Cancer 064008 | 2.9 |
| Ovarian Cancer (OD04768-07) | 3.8 |
| Ovary Margin (OD04768-08) | 0.2 |
| Normal Stomach | 1.2 |
| Gastric Cancer 9060358 | 0.8 |
| Stomach Margin 9060359 | 0.7 |
| Gastric Cancer 9060395 | 0.8 |
| Stomach Margin 9060394 | 0.2 |
| Gastric Cancer 9060397 | 0.5 |
| Stomach Margin 9060396 | 0.2 |
| Gastric Cancer 064005 | 2.5 |

Column A - Rel. Exp. (%) Ag1282, Run 170849610

TABLE BBE
Panel 4.1D

| Tissue Name | A |
|---|---|
| Secondary Th1 act | 12.0 |
| Secondary Th2 act | 19.3 |
| Secondary Tr1 act | 21.6 |
| Secondary Th1 rest | 7.5 |
| Secondary Th2 rest | 12.1 |
| Secondary Tr1 rest | 9.6 |
| Primary Th1 act | 20.0 |
| Primary Th2 act | 19.6 |
| Primary Tr1 act | 24.3 |
| Primary Th1 rest | 23.8 |
| Primary Th2 rest | 16.3 |
| Primary Tr1 rest | 54.0 |
| CD45RA CD4 lymphocyte act | 7.6 |
| CD45RO CD4 lymphocyte act | 0.0 |
| CD8 lymphocyte act | 23.3 |
| Secondary CD8 lymphocyte rest | 9.3 |
| Secondary CD8 lymphocyte act | 16.4 |
| CD4 lymphocyte none | 3.6 |
| 2ry Th1/Th2/Tr1_anti-CD95 CH11 | 23.7 |
| LAK cells rest | 7.9 |
| LAK cells IL-2 | 26.8 |
| LAK cells IL-2 + IL-12 | 5.5 |
| LAK cells IL-2 + IFN gamma | 10.8 |
| LAK cells IL-2 + IL-18 | 14.0 |
| LAK cells PMA/ionomycin | 1.5 |
| NK Cells IL-2 rest | 29.1 |
| Two Way MLR 3 day | 8.0 |
| Two Way MLR 5 day | 7.0 |
| Two Way MLR 7 day | 9.6 |
| PBMC rest | 0.3 |
| PBMC PWM | 4.0 |
| PBMC PHA-L | 12.7 |
| Ramos (B cell) none | 8.2 |
| Ramos (B cell) ionomycin | 11.5 |
| B lymphocytes PWM | 10.7 |
| B lymphocytes CD40L and IL-4 | 35.6 |
| EOL-1 dbcAMP | 9.2 |
| EOL-1 dbcAMP PMA/ionomycin | 10.3 |
| Dendritic cells none | 1.2 |
| Dendritic cells LPS | 0.7 |
| Dendritic cells anti-CD40 | 0.3 |
| Monocytes rest | 1.1 |
| Monocytes LPS | 2.4 |
| Macrophages rest | 2.2 |
| Macrophages LPS | 0.1 |
| HUVEC none | 1.1 |
| HUVEC starved | 3.6 |
| HUVEC IL-1beta | 2.6 |
| HUVEC IFN gamma | 2.6 |
| HUVEC TNF alpha + IFN gamma | 1.4 |
| HUVEC TNF alpha + IL4 | 3.1 |
| HUVEC IL-11 | 3.4 |
| Lung Microvascular EC none | 6.4 |
| Lung Microvascular EC TNFalpha + IL-1beta | 9.5 |
| Microvascular Dermal EC none | 10.4 |
| Microsvasular Dermal EC TNFalpha + IL-1beta | 10.2 |
| Bronchial epithelium TNFalpha + IL1beta | 3.4 |
| Small airway epithelium none | 0.0 |
| Small airway epithelium TNFalpha + IL-1beta | 2.7 |
| Coronery artery SMC rest | 0.3 |
| Coronery artery SMC TNFalpha + IL-1beta | 0.1 |
| Astrocytes rest | 1.1 |
| Astrocytes TNFalpha + IL-1beta | 2.4 |
| KU-812 (Basophil) rest | 14.5 |
| KU-812 (Basophil) PMA/ionomycin | 9.4 |
| CCD1106 (Keratinocytes) none | 4.6 |
| CCD1106 (Keratinocytes) TNFalpha + IL-1beta | 2.4 |
| Liver cirrhosis | 2.2 |
| NCI-H292 none | 10.6 |
| NCI-H292 IL-4 | 18.2 |
| NCI-H292 IL-9 | 24.5 |
| NCI-H292 IL-13 | 14.8 |
| NCI-H292 IFN gamma | 11.1 |
| HPAEC none | 3.3 |
| HPAEC TNF alpha + IL-1 beta | 5.3 |
| Lung fibroblast none | 1.1 |
| Lung fibroblast TNF alpha + IL-1 beta | 0.6 |
| Lung fibroblast IL-4 | 0.0 |
| Lung fibroblast IL-9 | 0.3 |
| Lung fibroblast IL-13 | 0.0 |
| Lung fibroblast IFN gamma | 0.0 |
| Dermal fibroblast CCD1070 rest | 5.4 |
| Dermal fibroblast CCD1070 TNF alpha | 24.3 |
| Dermal fibroblast CCD1070 IL-1 beta | 5.0 |
| Dermal fibroblast IFN gamma | 0.3 |
| Dermal fibroblast IL-4 | 1.6 |
| Dermal Fibroblasts rest | 0.8 |
| Neutrophils TNFa + LPS | 0.3 |
| Neutrophils rest | 0.6 |
| Colon | 8.4 |
| Lung | 5.1 |
| Thymus | 100.0 |
| Kidney | 1.3 |

Column A - Rel. Exp. (%) Ag1282, Run 169828985

TABLE BBF
Panel 5 Islet

| Tissue Name | A |
|---|---|
| 97457_Patient-02go_adipose | 42.6 |
| 97476_Patient-07sk_skeletal muscle | 37.9 |
| 97477_Patient-07ut_uterus | 15.8 |
| 97478_Patient-07pl_placenta | 85.9 |
| 99167_Bayer Patient 1 | 6.5 |
| 97482_Patient-08ut_uterus | 13.4 |
| 97483_Patient-08pl_placenta | 17.4 |
| 97486_Patient-09sk_skeletal muscle | 8.4 |
| 97487_Patient-09ut_uterus | 47.6 |
| 97488_Patient-09pl_placenta | 60.3 |
| 97492_Patient-10ut_uterus | 27.5 |
| 97493_Patient-10pl_placenta | 48.3 |
| 97495_Patient-11go_adipose | 27.5 |
| 97496_Patient-11sk_skeletal muscle | 2.9 |
| 97497_Patient-11ut_uterus | 29.7 |
| 97498_Patient-11pl_placenta | 11.8 |
| 97500_Patient-12go_adipose | 45.1 |
| 97501_Patient-12sk_skeletal muscle | 24.7 |
| 97502_Patient-12ut_uterus | 44.1 |
| 97503_Patient-12pl_placenta | 8.1 |
| 94721_Donor 2 U - A_Mesenchymal Stem Cells | 1.2 |
| 94722_Donor 2 U - B_Mesenchymal Stem Cells | 5.4 |
| 94723_Donor 2 U - C_Mesenchymal Stem Cells | 1.5 |
| 94709_Donor 2 AM - A_adipose | 11.9 |
| 94710_Donor 2 AM - B_adipose | 1.5 |
| 94711_Donor 2 AM - C_adipose | 1.5 |
| 94712_Donor 2 AD - A_adipose | 0.0 |
| 94713_Donor 2 AD - B_adipose | 1.5 |
| 94714_Donor 2 AD - C_adipose | 11.1 |
| 94742_Donor 3 U - A_Mesenchymal Stem Cells | 0.0 |
| 94743_Donor 3 U - B_Mesenchymal Stem Cells | 5.6 |
| 94730_Donor 3 AM - A_adipose | 0.0 |
| 94731_Donor 3 AM - B_adipose | 1.7 |
| 94732_Donor 3 AM - C_adipose | 3.9 |
| 94733_Donor 3 AD - A_adipose | 1.6 |
| 94734_Donor 3 AD - B_adipose | 1.8 |
| 94735_Donor 3 AD - C_adipose | 3.0 |
| 77138_Liver_HepG2untreated | 55.5 |
| 73556_Heart_Cardiac stromal cells (primary) | 85.3 |
| 81735_Small Intestine | 100.0 |
| 72409_Kidney_Proximal Convoluted Tubule | 23.7 |
| 82685_Small intestine_Duodenum | 31.4 |
| 90650_Adrenal_Adrenocortical adenoma | 0.0 |
| 72410_Kidney_HRCE | 22.8 |
| 72411_Kidney_HRE | 27.5 |
| 73139_Uterus_Uterine smooth muscle cells | 3.6 |

Column A - Rel. Exp. (%) Ag1282, Run 255155568

TABLE BBG
general oncology screening panel_v_2.4

| Tissue Name | A |
|---|---|
| Colon cancer 1 | 7.9 |
| Colon NAT 1 | 3.5 |
| Colon cancer 2 | 4.9 |
| Colon NAT 2 | 2.1 |
| Colon cancer 3 | 1.5 |
| Colon NAT 3 | 3.8 |
| Colon malignant cancer 4 | 18.7 |
| Colon NAT 4 | 4.0 |
| Lung cancer 1 | 7.1 |
| Lung NAT 1 | 2.5 |
| Lung cancer 2 | 100.0 |
| Lung NAT 2 | 1.6 |
| Squamous cell carcinoma 3 | 33.2 |
| Lung NAT 3 | 1.2 |
| Metastatic melanoma 1 | 22.8 |
| Melanoma 2 | 2.0 |
| Melanoma 3 | 5.7 |
| Metastatic melanoma 4 | 34.4 |
| Metastatic melanoma 5 | 62.9 |
| Bladder cancer 1 | 2.0 |
| Bladder NAT 1 | 0.0 |
| Bladder cancer 2 | 6.0 |
| Bladder NAT 2 | 1.2 |
| Bladder NAT 3 | 0.5 |
| Bladder NAT 4 | 8.1 |
| Prostate adenocarcinoma 1 | 56.6 |
| Prostate adenocarcinoma 2 | 2.1 |
| Prostate adenocarcinoma 3 | 4.3 |
| Prostate adenocarcinoma 4 | 4.1 |
| Prostate NAT 5 | 2.0 |
| Prostate adenocarcinoma 6 | 2.2 |
| Prostate adenocarcinoma 7 | 4.8 |
| Prostate adenocarcinoma 8 | 0.7 |
| Prostate adenocarcinoma 9 | 21.8 |
| Prostate NAT 10 | 0.8 |
| Kidney cancer 1 | 15.0 |
| Kidney NAT 1 | 3.1 |
| Kidney cancer 2 | 49.0 |
| Kidney NAT 2 | 2.9 |
| Kidney cancer 3 | 11.0 |
| Kidney NAT 3 | 1.9 |
| Kidney cancer 4 | 5.6 |
| Kidney NAT 4 | 0.2 |

Column A - Rel. Exp. (%) Ag1282, Run 267211473

General_screening_panel_v1.4 Summary: Ag1282 Highest expression of this gene was seen in a melanoma cell line. In addition, significantly higher levels of expression were seen in a breast cancer cell line. Thus, expression of this is useful as a marker to differentiate between these samples and other samples on this panel and as a marker to detect the presence of melanoma and breast cancer. Furthermore, therapeutic modulation of the expression or function of this gene or gene product is effective in the treatment of melanoma and breast cancers.

Among tissues with metabolic function, this gene was expressed at moderate levels in pituitary, adipose, adrenal gland, pancreas, thyroid, and adult and fetal skeletal muscle, heart, and liver. This widespread expression among these tissues shows that this gene product plays a role in normal neuroendocrine and metabolic function and that disregulated expression of this gene contributes to neuroendocrine disorders or metabolic diseases, such as obesity and diabetes.

In addition, this gene was expressed at much higher levels in fetal lung, liver and skeletal muscle tissue (CTs=27-29) when compared to expression in the adult counterpart (CTs=30-32). Thus, expression of this gene is useful as a marker to differentiate between the fetal and adult source of these tissue.

This molecule is a novel ovostatin that was also expressed at moderate levels in the all regions of the CNS examined. This gene or gene product is a target for the treatment of neurologic diseases.

HASS Panel v1.0 Summary: High levels of expression were detected in samples derived from a glioblastoma and a medulloblastoma.

Panel 1.3D Summary: Ag1282 Expression of this gene was consistent with expression in Panel 1.4. The expression of this gene was highest in a sample derived from a breast cancer cell line (MDA-N) (CT=26.9). In addition, there was expression in other samples derived from lung cancer cell lines and melanoma cell lines. Thus, the expression of this gene is useful as a marker to distinguish MDA-N cells from other samples in the panel. This gene encodes a novel ovostatin. Ovostatins are protease inhibitors that have been shown to support the growth of tumor cells in the absence of serum. They have also been shown to mediate accelerated fibroblast growth, collagen deposition and capillary formation. Thus, therapeutic targeting of this gene product is useful for blocking the uncontrolled growth of cancer cells related to the action of this gene, especially in those cancer types like lung, breast and melanoma tumors where the gene is overexpressed in the tumor compared to the normal adjacent tissue.

Panel 2D Summary: Ag1282 Highest expression of this gene was seen in a sample derived from an ocular melanoma metastasis to the liver (CT=27). In addition, there was expression in other samples derived from lung cancers. Thus, expression of this gene is useful as a marker to distinguish liver cancer cells from other samples in the panel. Moreover, therapeutic modulation of this gene, through the use of small molecule drugs, protein therapeutics or antibodies is of benefit in the treatment of liver or lung cancer.

Panel 4.1D Summary: Ag1282 This gene, an ovostatin-like protein, is related to ovostatin, a known inhibitor of proteinases of all four mechanistic classes, (serine proteinases, cysteine proteinases, aspartyl proteinases, and metalloproteinases) (Saxena I, Cell Mol Life Sci 1997 January;53(1):13-23; Ofuji Y, Periodontal Clin Investig 1992 Fall;14(2):13-22). Highest expression of the gene was seen in the thymus and kidney (CTs=28-29). In addition, moderate to low levels of expression were seen in most of the samples on this panel. This gene or its protein product is useful as a therapeutic protein for the reduction of various proteolytic activities involved in inflammatory and autoimmune diseases such as, but not limited to, Crohn's disease, ulcerative colitis, multiple sclerosis, chronic obstructive pulmonary disease, asthma, emphysema, rheumatoid arthritis, lupus erythematosus, or psoriasis, wound healing, and infection.

Panel 5 Islet Summary: Ag1282 Highest expression of this gene was seen in the small intestine (CT=28), with moderate to low levels of expression seen in metabolic tissues, including adipose, placenta, and skeletal muscle. Thus, targeting this gene or its protein product is useful in the treatment of metabolic disease, including obesity and type II diabetes.

general oncology screening panel_v_{—}2.4 Summary: Ag1282 Highest expression of this gene was seen in a lung cancer sample (CT=28). In addition, this gene was more highly expressed in lung and kidney cancer than in the corresponding normal adjacent tissue. Thus, therapeutic modulation of the expression or function of this gene product is useful in the treatment of lung and kidney cancer.

BC. CG59841-01: Novel Human Agrin

Expression of gene CG59841-01 was assessed using the primer-probe set Ag3605, described in Table BCA. Results of the RTQ-PCR runs are shown in Tables BCB, BCC, BCD and BCE.

TABLE BCA
Probe Name Ag3605

| | | | | SEQ |
|---|---|---|---|---|
| | | | Start | ID |
| Primers | Sequences | Length | Position | No |
| Forward | 5′-gaccccaagtcagaactgttc-3′ | 21 | 3174 | 1549 |
| Probe | TET-5′-attgagagcaccctggacgacctctt-3′- | 26 | 3213 | 1550 |
| | TAMRA | | | |
| Reverse | 5′-gaaatccttcttgacgtctgaa-3′ | 22 | 3245 | 1551 |

TABLE BCB
General_screening_panel_v1.4

| Tissue Name | A |
|---|---|
| Adipose | 1.4 |
| Melanoma* Hs688(A).T | 2.6 |
| Melanoma* Hs688(B).T | 4.6 |
| Melanoma* M14 | 6.7 |
| Melanoma* LOXIMVI | 4.8 |
| Melanoma* SK-MEL-5 | 2.6 |
| Squamous cell carcinoma SCC-4 | 9.0 |
| Testis Pool | 1.3 |
| Prostate ca.* (bone met) PC-3 | 21.0 |
| Prostate Pool | 0.9 |
| Placenta | 0.9 |
| Uterus Pool | 0.4 |
| Ovarian ca. OVCAR-3 | 77.4 |
| Ovarian ca. SK-OV-3 | 42.0 |
| Ovarian ca. OVCAR-4 | 9.5 |
| Ovarian ca. OVCAR-5 | 39.2 |
| Ovarian ca. IGROV-1 | 22.1 |
| Ovarian ca. OVCAR-8 | 18.0 |
| Ovary | 1.5 |
| Breast ca. MCF-7 | 7.7 |
| Breast ca. MDA-MB-231 | 22.7 |
| Breast ca. BT 549 | 13.2 |
| Breast ca. T47D | 100.0 |
| Breast ca. MDA-N | 4.8 |
| Breast Pool | 1.6 |
| Trachea | 2.8 |
| Lung | 0.2 |
| Fetal Lung | 11.4 |
| Lung ca. NCI-N417 | 1.4 |
| Lung ca. LX-1 | 10.5 |
| Lung ca. NCI-H146 | 0.1 |
| Lung ca. SHP-77 | 1.1 |
| Lung ca. A549 | 15.6 |
| Lung ca. NCI-H526 | 5.4 |
| Lung ca. NCI-H23 | 18.9 |
| Lung ca. NCI-H460 | 11.5 |
| Lung ca. HOP-62 | 23.7 |
| Lung ca. NCI-H522 | 1.8 |
| Liver | 0.5 |
| Fetal Liver | 0.8 |
| Liver ca. HepG2 | 15.5 |
| Kidney Pool | 1.5 |
| Fetal Kidney | 5.8 |
| Renal ca. 786-0 | 46.3 |
| Renal ca. A498 | 13.8 |
| Renal ca. ACHN | 14.3 |
| Renal ca. UO-31 | 41.5 |
| Renal ca. TK-10 | 19.3 |
| Bladder | 8.4 |
| Gastric ca. (liver met.) NCI-N87 | 87.7 |
| Gastric ca. KATO III | 17.8 |
| Colon ca. SW-948 | 9.2 |
| Colon ca. SW480 | 25.0 |
| Colon ca.* (SW480 met) SW620 | 5.0 |
| Colon ca. HT29 | 26.8 |
| Colon ca. HCT-116 | 4.9 |
| Colon ca. CaCo-2 | 13.6 |
| Colon cancer tissue | 10.2 |
| Colon ca. SW1116 | 5.1 |
| Colon ca. Colo-205 | 1.8 |
| Colon ca. SW-48 | 1.2 |
| Colon Pool | 1.9 |
| Small Intestine Pool | 0.6 |
| Stomach Pool | 1.5 |
| Bone Marrow Pool | 0.6 |
| Fetal Heart | 1.4 |
| Heart Pool | 0.7 |
| Lymph Node Pool | 2.1 |
| Fetal Skeletal Muscle | 0.9 |
| Skeletal Muscle Pool | 0.4 |
| Spleen Pool | 0.7 |
| Thymus Pool | 1.8 |
| CNS cancer (glio/astro) U87-MG | 5.9 |
| CNS cancer (glio/astro) U-118-MG | 11.7 |
| CNS cancer (neuro; met) SK-N-AS | 1.2 |
| CNS cancer (astro) SF-539 | 6.7 |
| CNS cancer (astro) SNB-75 | 22.8 |
| CNS cancer (glio) SNB-19 | 25.0 |
| CNS cancer (glio) SF-295 | 35.1 |
| Brain (Amygdala) Pool | 1.7 |
| Brain (cerebellum) | 1.4 |
| Brain (fetal) | 7.0 |
| Brain (Hippocampus) Pool | 1.6 |
| Cerebral Cortex Pool | 1.9 |
| Brain (Substantia nigra) Pool | 2.8 |
| Brain (Thalamus) Pool | 2.7 |
| Brain (whole) | 3.4 |
| Spinal Cord Pool | 1.8 |
| Adrenal Gland | 0.2 |
| Pituitary gland Pool | 0.3 |
| Salivary Gland | 1.1 |
| Thyroid (female) | 3.3 |
| Pancreatic ca. CAPAN2 | 23.7 |
| Pancreas Pool | 3.0 |

Column A - Rel. Exp. (%) Ag3605, Run 213406184

TABLE BCC
Panel 2.2

| Tissue Name | A |
|---|---|
| Normal Colon | 4.7 |
| Colon cancer (OD06064) | 6.3 |
| Colon Margin (OD06064) | 2.3 |
| Colon cancer (OD06159) | 2.1 |
| Colon Margin (OD06159) | 1.8 |
| Colon cancer (OD06297-04) | 2.0 |
| Colon Margin (OD06297-05) | 3.0 |
| CC Gr.2 ascend colon (ODO3921) | 3.6 |
| CC Margin (ODO3921) | 1.3 |
| Colon cancer metastasis (OD06104) | 1.1 |
| Lung Margin (OD06104) | 1.0 |
| Colon mets to lung (OD04451-01) | 4.5 |
| Lung Margin (OD04451-02) | 6.7 |
| Normal Prostate | 2.1 |
| Prostate Cancer (OD04410) | 3.9 |
| Prostate Margin (OD04410) | 3.4 |
| Normal Ovary | 7.6 |
| Ovarian cancer (OD06283-03) | 27.9 |
| Ovarian Margin (OD06283-07) | 2.0 |
| Ovarian Cancer 064008 | 16.0 |
| Ovarian cancer (OD06145) | 10.1 |
| Ovarian Margin (OD06145) | 8.2 |
| Ovarian cancer (OD06455-03) | 28.9 |
| Ovarian Margin (OD06455-07) | 1.9 |
| Normal Lung | 2.9 |
| Invasive poor diff. lung adeno (ODO4945-01 | 9.4 |
| Lung Margin (ODO4945-03) | 7.9 |
| Lung Malignant Cancer (OD03126) | 7.5 |
| Lung Margin (OD03126) | 7.0 |
| Lung Cancer (OD05014A) | 17.0 |
| Lung Margin (OD05014B) | 11.7 |
| Lung cancer (OD06081) | 12.2 |
| Lung Margin (OD06081) | 2.4 |
| Lung Cancer (OD04237-01) | 1.8 |
| Lung Margin (OD04237-02) | 16.2 |
| Ocular Melanoma Metastasis | 8.4 |
| Ocular Melanoma Margin (Liver) | 2.9 |
| Melanoma Metastasis | 4.0 |
| Melanoma Margin (Lung) | 3.8 |
| Normal Kidney | 10.9 |
| Kidney Ca, Nuclear grade 2 (OD04338) | 35.4 |
| Kidney Margin (OD04338) | 20.4 |
| Kidney Ca Nuclear grade 1/2 (OD04339) | 52.9 |
| Kidney Margin (OD04339) | 16.6 |
| Kidney Ca, Clear cell type (OD04340) | 16.6 |
| Kidney Margin (OD04340) | 7.4 |
| Kidney Ca, Nuclear grade 3 (OD04348) | 11.2 |
| Kidney Margin (OD04348) | 100.0 |
| Kidney malignant cancer (OD06204B) | 21.6 |
| Kidney normal adjacent tissue (OD06204E) | 23.2 |
| Kidney Cancer (OD04450-01) | 53.2 |
| Kidney Margin (OD04450-03) | 21.3 |
| Kidney Cancer 8120613 | 1.1 |
| Kidney Margin 8120614 | 14.1 |
| Kidney Cancer 9010320 | 20.3 |
| Kidney Margin 9010321 | 15.0 |
| Kidney Cancer 8120607 | 71.7 |
| Kidney Margin 8120608 | 12.2 |
| Normal Uterus | 7.3 |
| Uterine Cancer 064011 | 6.9 |
| Normal Thyroid | 4.3 |
| Thyroid Cancer 064010 | 27.0 |
| Thyroid Cancer A302152 | 19.1 |
| Thyroid Margin A302153 | 8.1 |
| Normal Breast | 14.0 |
| Breast Cancer (OD04566) | 13.4 |
| Breast Cancer 1024 | 35.1 |
| Breast Cancer (OD04590-01) | 31.6 |
| Breast Cancer Mets (OD04590-03) | 8.7 |
| Breast Cancer Metastasis (OD04655-05) | 13.3 |
| Breast Cancer 064006 | 21.5 |
| Breast Cancer 9100266 | 17.7 |
| Breast Margin 9100265 | 16.5 |
| Breast Cancer A209073 | 13.2 |
| Breast Margin A2090734 | 35.4 |
| Breast cancer (OD06083) | 24.5 |
| Breast cancer node metastasis (OD06083) | 21.5 |
| Normal Liver | 5.0 |
| Liver Cancer 1026 | 15.5 |
| Liver Cancer 1025 | 12.2 |
| Liver Cancer 6004-T | 7.8 |
| Liver Tissue 6004-N | 6.1 |
| Liver Cancer 6005-T | 25.0 |
| Liver Tissue 6005-N | 12.4 |
| Liver Cancer 064003 | 12.9 |
| Normal Bladder | 14.2 |
| Bladder Cancer 1023 | 9.5 |
| Bladder Cancer A302173 | 12.2 |
| Normal Stomach | 8.7 |
| Gastric Cancer 9060397 | 8.9 |
| Stomach Margin 9060396 | 7.4 |
| Gastric Cancer 9060395 | 7.0 |
| Stomach Margin 9060394 | 7.5 |
| Gastric Cancer 064005 | 6.9 |

Column A - Rel. Exp. (%) Ag3605, Run 173764229

TABLE BCD
Panel 4.1D

| Tissue Name | A |
|---|---|
| Secondary Th1 act | 1.0 |
| Secondary Th2 act | 5.1 |
| Secondary Tr1 act | 2.5 |
| Secondary Th1 rest | 0.0 |
| Secondary Th2 rest | 0.4 |
| Secondary Tr1 rest | 0.4 |
| Primary Th1 act | 3.6 |
| Primary Th2 act | 1.1 |
| Primary Tr1 act | 3.4 |
| Primary Th1 rest | 0.9 |
| Primary Th2 rest | 0.5 |
| Primary Tr1 rest | 0.2 |
| CD45RA CD4 lymphocyte act | 43.5 |
| CD45RO CD4 lymphocyte act | 5.0 |
| CD8 lymphocyte act | 3.9 |
| Secondary CD8 lymphocyte rest | 3.7 |
| Secondary CD8 lymphocyte act | 3.3 |
| CD4 lymphocyte none | 0.3 |
| 2ry Th1/Th2/Tr1_anti-CD95 CH11 | 0.3 |
| LAK cells rest | 5.0 |
| LAK cells IL-2 | 2.8 |
| LAK cells IL-2 + IL-12 | 1.4 |
| LAK cells IL-2 + IFN gamma | 2.3 |
| LAK cells IL-2 + IL-18 | 2.9 |
| LAK cells PMA/ionomycin | 6.8 |
| NK Cells IL-2 rest | 1.6 |
| Two Way MLR 3 day | 10.7 |
| Two Way MLR 5 day | 6.5 |
| Two Way MLR 7 day | 4.3 |
| PBMC rest | 0.0 |
| PBMC PWM | 5.0 |
| PBMC PHA-L | 5.5 |
| Ramos (B cell) none | 0.4 |
| Ramos (B cell) ionomycin | 0.2 |
| B lymphocytes PWM | 2.4 |
| B lymphocytes CD40L and IL-4 | 1.5 |
| EOL-1 dbcAMP | 3.4 |
| EOL-1 dbcAMP PMA/ionomycin | 18.3 |
| Dendritic cells none | 14.8 |
| Dendritic cells LPS | 48.3 |
| Dendritic cells anti-CD40 | 9.7 |
| Monocytes rest | 0.9 |
| Monocytes LPS | 66.4 |
| Macrophages rest | 16.2 |
| Macrophages LPS | 54.7 |
| HUVEC none | 9.3 |
| HUVEC starved | 14.4 |
| HUVEC IL-1beta | 15.6 |
| HUVEC IFN gamma | 12.9 |
| HUVEC TNF alpha + IFN gamma | 37.6 |
| HUVEC TNF alpha + IL4 | 31.4 |
| HUVEC IL-11 | 14.9 |
| Lung Microvascular EC none | 79.0 |
| Lung Microvascular EC TNFalpha + IL-1beta | 100.0 |
| Microvascular Dermal EC none | 49.7 |
| Microsvasular Dermal EC TNFalpha + IL-1beta | 56.6 |
| Bronchial epithelium TNFalpha + IL1beta | 78.5 |
| Small airway epithelium none | 31.0 |
| Small airway epithelium TNFalpha + IL-1beta | 81.8 |
| Coronery artery SMC rest | 17.2 |
| Coronery artery SMC TNFalpha + IL-1beta | 22.2 |
| Astrocytes rest | 80.1 |
| Astrocytes TNFalpha + IL-1beta | 82.9 |
| KU-812 (Basophil) rest | 2.6 |
| KU-812 (Basophil) PMA/ionomycin | 0.6 |
| CCD1106 (Keratinocytes) none | 70.7 |
| CCD1106 (Keratinocytes) TNFalpha + IL-1beta | 77.4 |
| Liver cirrhosis | 13.2 |
| NCI-H292 none | 57.8 |
| NCI-H292 IL-4 | 62.4 |
| NCI-H292 IL-9 | 61.6 |
| NCI-H292 IL-13 | 53.6 |
| NCI-H292 IFN gamma | 67.4 |
| HPAEC none | 21.5 |
| HPAEC TNF alpha + IL-1 beta | 37.6 |
| Lung fibroblast none | 22.4 |
| Lung fibroblast TNF alpha + IL-1 beta | 71.2 |
| Lung fibroblast IL-4 | 16.2 |
| Lung fibroblast IL-9 | 31.9 |
| Lung fibroblast IL-13 | 18.7 |
| Lung fibroblast IFN gamma | 23.0 |
| Dermal fibroblast CCD1070 rest | 15.9 |
| Dermal fibroblast CCD1070 TNF alpha | 15.9 |
| Dermal fibroblast CCD1070 IL-1 beta | 17.2 |
| Dermal fibroblast IFN gamma | 7.2 |
| Dermal fibroblast IL-4 | 7.2 |
| Dermal Fibroblasts rest | 4.3 |
| Neutrophils TNFa + LPS | 0.0 |
| Neutrophils rest | 0.2 |
| Colon | 7.0 |
| Lung | 23.3 |
| Thymus | 5.8 |
| Kidney | 23.2 |

Column A - Rel. Exp. (%) Ag3605, Run 169943454

TABLE BCE
general oncology screening panel_v_2.4

| Tissue Name | A |
|---|---|
| Colon cancer 1 | 11.6 |
| Colon cancer NAT 1 | 5.3 |
| Colon cancer 2 | 18.6 |
| Colon cancer NAT 2 | 3.2 |
| Colon cancer 3 | 22.2 |
| Colon cancer NAT 3 | 7.7 |
| Colon malignant cancer 4 | 23.5 |
| Colon normal adjacent tissue 4 | 2.0 |
| Lung cancer 1 | 57.0 |
| Lung NAT 1 | 3.1 |
| Lung cancer 2 | 100.0 |
| Lung NAT 2 | 3.9 |
| Squamous cell carcinoma 3 | 41.2 |
| Lung NAT 3 | 1.7 |
| metastatic melanoma 1 | 5.4 |
| Melanoma 2 | 3.2 |
| Melanoma 3 | 3.7 |
| metastatic melanoma 4 | 12.5 |
| metastatic melanoma 5 | 7.9 |
| Bladder cancer 1 | 0.5 |
| Bladder cancer NAT 1 | 0.0 |
| Bladder cancer 2 | 6.0 |
| Bladder cancer NAT 2 | 0.2 |
| Bladder cancer NAT 3 | 1.7 |
| Bladder cancer NAT 4 | 1.8 |
| Prostate adenocarcinoma 1 | 2.4 |
| Prostate adenocarcinoma 2 | 2.6 |
| Prostate adenocarcinoma 3 | 5.2 |
| Prostate adenocarcinoma 4 | 21.9 |
| Prostate cancer NAT 5 | 5.4 |
| Prostate adenocarcinoma 6 | 4.4 |
| Prostate adenocarcinoma 7 | 3.8 |
| Prostate adenocarcinoma 8 | 1.8 |
| Prostate adenocarcinoma 9 | 10.7 |
| Prostate cancer NAT 10 | 0.7 |
| Kidney cancer 1 | 38.7 |
| Kidney NAT 1 | 20.6 |
| Kidney cancer 2 | 66.4 |
| Kidney NAT 2 | 39.8 |
| Kidney cancer 3 | 64.6 |
| Kidney NAT 3 | 19.6 |
| Kidney cancer 4 | 16.4 |
| Kidney NAT 4 | 15.6 |

Column A - Rel. Exp. (%) Ag3605, Run 260268655

General_screening_panel_v1.4 Summary: Ag3605 Expression of this gene was highest in a breast cancer cell line (CT=25.2). Expression of this gene was primarily associated with cancer cell lines rather than with normal tissues and was upregulated in pancreatic, CNS, colon, gastric, renal, lung, breast, ovarian, and prostate cancer cell lines when compared to their respective normal tissues. Thus, therapeutic modulation of the activity of this gene or its protein product, using small molecule drugs, antibodies or protein therapeutics, is of benefit in the treatment of these types of cancers.

In addition, this gene was expressed at moderate levels in all regions of the central nervous system examined, including amygdala, hippocampus, substantia nigra, thalamus, cerebellum, cerebral cortex, and spinal cord. This gene encodes a protein with homology to agrin, a neuronal aggregating factor that induces the aggregation of acetylcholine receptors and other postsynaptic proteins on muscle fibers and is crucial for the formation of the neuromuscular junction. Arin plays an important role in defining neuronal responses to excitatory neurotransmitters both in vitro and in vivo (Hilgenberg LG, Mol Cell Neurosci 2002 January;19(1):97-110; Bixby J L,. J Neurobiol 2002 February 5;50(2):164-79). This gene expression in the central nervous system was consistent with the hypothesis that this protein functions as agrin. Therefore, this gene plays a role in central nervous system disorders such as Alzheimer's disease, Parkinson's disease, epilepsy, multiple sclerosis, schizophrenia and depression.

Among tissues with metabolic or endocrine function, this gene was expressed at moderate levels in pancreas, adipose, thyroid, and the gastrointestinal tract and at low levels in adrenal gland, pituitary gland, skeletal muscle, heart, and liver. Therefore, therapeutic modulation of the activity of this gene is useful in the treatment of endocrine/metabolically related diseases, such as obesity and diabetes.

Panel 2.2 Summary: Ag3605 Expression of this gene was highest in a sample of normal kidney (CT=27.4). In addition, expression of this gene was upregulated in a number of ovarian and renal cancers when compared to the matched control margins. Expression of this gene is useful as a marker for ovarian and renal carcinoma. Therapeutic modulation of the activity of this gene or its protein product, using small molecule drugs, antibodies or protein therapeutics, is of benefit in the treatment of renal and ovarian cancer.

Panel 4.1D Summary: Ag3605 Expression of this gene was highest in lung microvascular endothelial cells, microvascular dermal endothelial cells, mucoepidermoid cell line NCI-H292, astrocytes, and keratinocytes. Therefore, small molecule drug, antibody or protein therapeutics designed against the protein encoded by this gene is useful for the reduction or inhibition of inflammation in asthma, emphysema, allergy, psoriasis, muscular dystrophy and multiple sclerosis.

general oncology screening panel_V_{—}2.4 Summary: Ag3605 Highest expression of this gene was seen in lung cancer (CT=26.8). Higher levels of expression were seen in lung and kidney cancers when compared to expression in normal adjacent tissue. Expression of this gene is useful as a marker of lung and kidney cancers. Therapeutic modulation of the expression or function of this gene product is useful in the treatment of lung and kidney cancers.

BD. CG59905-01 and CG59905-02: Sushi Containing Membrane Protein

Expression of gene CG59905-01 and variant CG59905-02 was assessed using the primer-probe set Ag2443, described in Table BDA. Results of the RTQ-PCR runs are

TABLE BDA
Probe Name Ag2443

| | | | | SEQ |
|---|---|---|---|---|
| | | | Start | ID |
| Primers | Sequences | Length | Position | No |
| Forward | 5′-cagtttgggaccgacttca-3′ | 19 | 652 | 1552 |
| Probe | TET-5′-caagactgtgagctatcagtgtaaccca-3′- | 28 | 678 | 1553 |
| | TAMRA | | | |
| Reverse | 5′-tgactgcttccatgacatagc-3′ | 21 | 707 | 1554 |

TABLE BDB
Panel 1.3D

| Tissue Name | A |
|---|---|
| Liver adenocarcinoma | 16.6 |
| Pancreas 1 | 0.0 |
| Pancreatic ca. CAPAN 2 | 0.0 |
| Adrenal gland | 1.5 |
| Thyroid | 0.0 |
| Salivary gland | 0.0 |
| Pituitary gland | 2.2 |
| Brain (fetal) | 51.4 |
| Brain (whole) | 92.0 |
| Brain (amygdala) | 52.1 |
| Brain (cerebellum) | 47.6 |
| Brain (hippocampus) | 78.5 |
| Brain (substantia nigra) | 8.0 |
| Brain (thalamus) | 12.9 |
| Cerebral Cortex | 100.0 |
| Spinal cord | 6.5 |
| glio/astro U87-MG | 0.0 |
| glio/astro U-118-MG | 0.0 |
| astrocytoma SW1783 | 0.0 |
| neuro*; met SK-N-AS | 0.0 |
| astrocytoma SF-539 | 0.0 |
| astrocytoma SNB-75 | 0.0 |
| glioma SNB-19 | 0.6 |
| glioma U251 | 29.9 |
| glioma SF-295 | 1.9 |
| Heart (fetal) | 0.0 |
| Heart | 0.0 |
| Skeletal muscle (fetal) | 6.2 |
| Skeletal muscle | 0.8 |
| Bone marrow | 0.0 |
| Thymus | 0.0 |
| Spleen | 5.4 |
| Lymph node | 0.5 |
| Colorectal | 0.7 |
| Stomach | 0.0 |
| Small intestine | 1.5 |
| Colon ca. SW480 | 0.0 |
| Colon ca.* SW620(SW480 met) | 0.0 |
| Colon ca. HT29 | 0.0 |
| Colon ca. HCT-116 | 0.0 |
| Colon ca. CaCo-2 | 0.0 |
| Colon ca. tissue(ODO3866) | 0.0 |
| Colon ca. HCC-2998 | 0.0 |
| Gastric ca.* (liver met) NCI-N87 | 0.0 |
| Bladder | 1.2 |
| Trachea | 2.0 |
| Kidney | 4.1 |
| Kidney (fetal) | 2.5 |
| Renal ca. 786-0 | 0.0 |
| Renal ca. A498 | 0.0 |
| Renal ca. RXF 393 | 0.0 |
| Renal ca. ACHN | 0.0 |
| Renal ca. UO-31 | 0.0 |
| Renal ca. TK-10 | 0.0 |
| Liver | 0.0 |
| Liver (fetal) | 0.0 |
| Liver ca. (hepatoblast) HepG2 | 0.0 |
| Lung | 0.0 |
| Lung (fetal) | 1.4 |
| Lung ca. (small cell) LX-1 | 0.0 |
| Lung ca. (small cell) NCI-H69 | 0.0 |
| Lung ca. (s. cell var.) SHP-77 | 16.8 |
| Lung ca. (large cell)NCI-H460 | 1.6 |
| Lung ca. (non-sm. cell) A549 | 0.0 |
| Lung ca. (non-s. cell) NCI-H23 | 11.2 |
| Lung ca. (non-s. cell) HOP-62 | 0.0 |
| Lung ca. (non-s. cl) NCI-H522 | 0.0 |
| Lung ca. (squam.) SW 900 | 0.0 |
| Lung ca. (squam.) NCI-H596 | 4.8 |
| Mammary gland | 2.1 |
| Breast ca.* (pl. ef) MCF-7 | 0.0 |
| Breast ca.* (pl. ef) MDA-MB-231 | 0.0 |
| Breast ca.* (pl. ef) T47D | 10.9 |
| Breast ca. BT-549 | 0.0 |
| Breast ca. MDA-N | 9.3 |
| Ovary | 1.1 |
| Ovarian ca. OVCAR-3 | 11.5 |
| Ovarian ca. OVCAR-4 | 0.0 |
| Ovarian ca. OVCAR-5 | 0.0 |
| Ovarian ca. OVCAR-8 | 0.0 |
| Ovarian ca. IGROV-1 | 0.0 |
| Ovarian ca.* (ascites) SK-OV-3 | 0.8 |
| Uterus | 2.8 |
| Placenta | 2.9 |
| Prostate | 0.9 |
| Prostate ca.* (bone met)PC-3 | 0.0 |
| Testis | 20.9 |
| Melanoma Hs688(A).T | 0.0 |
| Melanoma* (met) Hs688(B).T | 0.6 |
| Melanoma UACC-62 | 19.5 |
| Melanoma M14 | 15.1 |
| Melanoma LOX IMVI | 0.7 |
| Melanoma* (met) SK-MEL-5 | 7.3 |
| Adipose | 0.4 |

Column A - Rel. Exp. (%) Ag2443, Run 165517738

TABLE BDC
Panel 2D

| Tissue Name | A |
|---|---|
| Normal Colon | 12.6 |
| CC Well to Mod Diff (ODO3866) | 1.2 |
| CC Margin (ODO3866) | 2.9 |
| CC Gr.2 rectosigmoid (ODO3868) | 1.9 |
| CC Margin (ODO3868) | 3.2 |
| CC Mod Diff (ODO3920) | 1.5 |
| CC Margin (ODO3920) | 2.6 |
| CC Gr.2 ascend colon (ODO3921) | 9.3 |
| CC Margin (ODO3921) | 2.9 |
| CC from Partial Hepatectomy (ODO4309) Mets | 0.0 |
| Liver Margin (ODO4309) | 0.0 |
| Colon mets to lung (OD04451-01) | 2.5 |
| Lung Margin (OD04451-02) | 18.0 |
| Normal Prostate 6546-1 | 4.2 |
| Prostate Cancer (OD04410) | 0.0 |
| Prostate Margin (OD04410) | 5.3 |
| Prostate Cancer (OD04720-01) | 6.0 |
| Prostate Margin (OD04720-02) | 4.5 |
| Normal Lung 061010 | 29.9 |
| Lung Met to Muscle (ODO4286) | 2.4 |
| Muscle Margin (ODO4286) | 3.1 |
| Lung Malignant Cancer (OD03126) | 12.2 |
| Lung Margin (OD03126) | 20.4 |
| Lung Cancer (OD04404) | 3.0 |
| Lung Margin (OD04404) | 30.1 |
| Lung Cancer (OD04565) | 0.8 |
| Lung Margin (OD04565) | 9.0 |
| Lung Cancer (OD04237-01) | 7.8 |
| Lung Margin (OD04237-02) | 3.1 |
| Ocular Mel Met to Liver (ODO4310) | 3.2 |
| Liver Margin (ODO4310) | 1.1 |
| Melanoma Mets to Lung (OD04321) | 0.0 |
| Lung Margin (OD04321) | 17.2 |
| Normal Kidney | 100.0 |
| Kidney Ca, Nuclear grade 2 (OD04338) | 0.0 |
| Kidney Margin (OD04338) | 16.8 |
| Kidney Ca Nuclear grade 1/2 (OD04339) | 6.3 |
| Kidney Margin (OD04339) | 59.0 |
| Kidney Ca, Clear cell type (OD04340) | 10.7 |
| Kidney Margin (OD04340) | 38.4 |
| Kidney Ca, Nuclear grade 3 (OD04348) | 0.8 |
| Kidney Margin (OD04348) | 18.4 |
| Kidney Cancer (OD04622-01) | 1.8 |
| Kidney Margin (OD04622-03) | 8.9 |
| Kidney Cancer (OD04450-01) | 1.6 |
| Kidney Margin (OD04450-03) | 21.3 |
| Kidney Cancer 8120607 | 0.0 |
| Kidney Margin 8120608 | 37.1 |
| Kidney Cancer 8120613 | 0.0 |
| Kidney Margin 8120614 | 31.6 |
| Kidney Cancer 9010320 | 4.2 |
| Kidney Margin 9010321 | 62.9 |
| Normal Uterus | 1.4 |
| Uterus Cancer 064011 | 19.6 |
| Normal Thyroid | 0.6 |
| Thyroid Cancer 064010 | 6.6 |
| Thyroid Cancer A302152 | 0.0 |
| Thyroid Margin A302153 | 1.9 |
| Normal Breast | 22.2 |
| Breast Cancer (OD04566) | 0.8 |
| Breast Cancer (OD04590-01) | 2.6 |
| Breast Cancer Mets (OD04590-03) | 2.0 |
| Breast Cancer Metastasis (OD04655-05) | 2.9 |
| Breast Cancer 064006 | 4.3 |
| Breast Cancer 1024 | 6.5 |
| Breast Cancer 9100266 | 6.5 |
| Breast Margin 9100265 | 15.7 |
| Breast Cancer A209073 | 10.7 |
| Breast Margin A209073 | 31.4 |
| Normal Liver | 0.0 |
| Liver Cancer 064003 | 26.6 |
| Liver Cancer 1025 | 0.0 |
| Liver Cancer 1026 | 0.0 |
| Liver Cancer 6004-T | 0.0 |
| Liver Tissue 6004-N | 29.1 |
| Liver Cancer 6005-T | 0.0 |
| Liver Tissue 6005-N | 0.0 |
| Normal Bladder | 0.4 |
| Bladder Cancer 1023 | 0.0 |
| Bladder Cancer A302173 | 39.0 |
| Bladder Cancer (OD04718-01) | 0.0 |
| Bladder Normal Adjacent (OD04718-03) | 4.5 |
| Normal Ovary | 2.6 |
| Ovarian Cancer 064008 | 0.0 |
| Ovarian Cancer (OD04768-07) | 0.0 |
| Ovary Margin (OD04768-08) | 0.0 |
| Normal Stomach | 2.2 |
| Gastric Cancer 9060358 | 0.0 |
| Stomach Margin 9060359 | 1.3 |
| Gastric Cancer 9060395 | 4.6 |
| Stomach Margin 9060394 | 5.3 |
| Gastric Cancer 9060397 | 2.5 |
| Stomach Margin 9060396 | 0.8 |
| Gastric Cancer 064005 | 0.0 |

Column A - Rel. Exp. (%) Ag2443, Run 164988396

TABLE BDD
Panel 4D

| Tissue Name | A |
|---|---|
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.1 |
| PBMC PWM | 0.0 |
| PBMC PHA-L | 0.0 |
| Ramos (B cell) none | 35.6 |
| Ramos (B cell) ionomycin | 100.0 |
| B lymphocytes PWM | 0.0 |
| B lymphocytes CD40L and IL-4 | 0.0 |
| EOL-1 dbcAMP | 0.5 |
| EOL-1 dbcAMP PMA/ionomycin | 0.0 |
| Dendritic cells none | 0.2 |
| Dendritic cells LPS | 0.0 |
| Dendritic cells anti-CD40 | 0.0 |
| Monocytes rest | 0.1 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 0.7 |
| KU-812 (Basophil) PMA/ionomycin | 0.4 |
| CCD1106 (Keratinocytes) none | 0.0 |
| CCD1106 (Keratinocytes) TNFalpha + IL-1beta | 0.0 |
| Liver cirrhosis | 0.2 |
| Lupus kidney | 0.1 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 0.2 |
| HPAEC TNF alpha + IL-1 beta | 0.1 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| IBD Colitis 2 | 0.0 |
| IBD Crohn's | 0.0 |
| Colon | 0.1 |
| Lung | 0.1 |
| Thymus | 2.5 |
| Kidney | 0.1 |

Column A - Rel. Exp. (%) Ag2443, Run 164337611

Panel 1.3D Summary: Ag2443 Highest expression of this gene was detected in the cerebral cortex (CT=30.9). In addition, high expression of this gene was observed exclusively in all the brain regions (CTs=31). Expression of this gene is useful as a marker to distinguish these brain samples from other samples used in this panel. Low but significant expression was also seen in spinal cord region. Therefore, therapeutic modulation of the function or activity of this gene or its protein product, through the use of small molecule drugs, protein therapeutics or antibodies, is beneficial in the treatment of central nervous system disorders such as Alzheimer's disease, Parkinson's disease, epilepsy, multiple sclerosis, schizophrenia and depression.

This gene encodes a variant of cub and sushi multiple domains 1 protein (CSMD1). A related protein, SEZ-6, has recently been shown to be associated with neuronal bursting activity of seizures (Shimizu-Nishikawa K, Brain Res Mol Brain Res 28(2):201-10). Thus, the protein encoded by this gene also plays a role in neuronal siezures.

In addition, low to moderate expression of this gene was seen in melanoma, ovarian cancer OVCAR-3, breast cancer, lung cancer, liver adenocarcinoma and glioma U25 1 cell lines. Therapeutic modulation of the activity of this gene or its protein product, through the use of small molecule drugs, or antibodies, is beneficial in the treatment of these cancers.

Significant expression was also detected in fetal skeletal muscle.

Panel 2D Summary: Ag2443 Highest expression of this gene was detected in a kidney sample (CT=31). Expression of this gene was down-regulated in kidney and lung cancer. In addition, there was substantial expression in other samples derived from bladder cancer, liver cancer, uterine cancer, breast cancer, kidney cancer and lung cancer. This gene encodes a variant of cub and sushi multiple domains 1 protein (CSMD1). Recently, CSMD1 has been shown to be a candidate for tumor suppressor of multiple types of cancer (Sun PC. Genomics 75(1-3):17-25). Therefore, therapeutic modulation of the activity of this gene or its protein product, through the use of small molecule drugs, or antibodies, is beneficial in the treatment of bladder, liver, uterine, breast, kidney and lung cancers.

Panel 4D Summary: Ag2443 Highest expression of this gene was seen in Ramos B cells (CTs=26-27). Expression of this gene is useful as a marker to distinguish these samples from other samples in this panel. In addition, expression of this gene in B cells shows that this gene is involved in rheumatic disease including rheumatoid arthritis, lupus, osteoarthritis, and hyperproliferative B cell disorders.

Low but significant expression of this gene was also detected in thymus. Drugs that inhibit the function of this protein are useful in the regulation of T cell development in the thymus and for the reduction or elimination of the symptoms of T cell mediated autoimmune or inflammatory diseases, including asthma, allergies, inflammatory bowel disease, lupus erythematosus, or rheumatoid arthritis. Small molecule or antibody therapeutics designed against this putative protein are useful for the disruption of T cell development in the thymus and as immunosuppresants for tissue transplant.

BE. CG9271501: KIAA0918

Expression of gene CG92715-01 was assessed using the primer-probe set Ag2502, described in Table BEA. Results of the RTQ-PCR runs are shown in Tables BEB, BEC, BED and BEE.

TABLE BEA
Probe Name Ag2502

| | | | | SEQ |
|---|---|---|---|---|
| | | | Start | ID |
| Primers | Sequences | Length | Position | No |
| Forward | 5′-ggagtaaccacttcacctcctt-3′ | 22 | 1632 | 1555 |
| Probe | TET-5′-ccagctgaagtcactcatccaaatcg-3′- | 26 | 1675 | 1556 |
| | TAMRA | | | |
| Reverse | 5′-aggtacaatcccaaggattgtc-3′ | 22 | 1709 | 1557 |

TABLE BEB
Panel 1.3D

| Tissue Name | A |
|---|---|
| Liver adenocarcinoma | 4.5 |
| Pancreas | 1.1 |
| Pancreatic ca. CAPAN 2 | 14.9 |
| Adrenal gland | 0.8 |
| Thyroid | 10.1 |
| Salivary gland | 17.3 |
| Pituitary gland | 2.9 |
| Brain (fetal) | 76.3 |
| Brain (whole) | 95.3 |
| Brain (amygdala) | 80.1 |
| Brain (cerebellum) | 61.1 |
| Brain (hippocampus) | 100.0 |
| Brain (substantia nigra) | 16.2 |
| Brain (thalamus) | 25.0 |
| Cerebral Cortex | 92.0 |
| Spinal cord | 20.0 |
| glio/astro U87-MG | 24.7 |
| glio/astro U-118-MG | 22.4 |
| astrocytoma SW1783 | 0.2 |
| neuro*; met SK-N-AS | 42.3 |
| astrocytoma SF-539 | 0.0 |
| astrocytoma SNB-75 | 3.6 |
| glioma SNB-19 | 2.5 |
| glioma U251 | 19.5 |
| glioma SF-295 | 0.0 |
| Heart (fetal) | 1.3 |
| Heart | 0.8 |
| Skeletal muscle (fetal) | 8.2 |
| Skeletal muscle | 0.0 |
| Bone marrow | 0.0 |
| Thymus | 0.7 |
| Spleen | 0.5 |
| Lymph node | 0.3 |
| Colorectal | 0.7 |
| Stomach | 3.1 |
| Small intestine | 2.4 |
| Colon ca. SW480 | 0.2 |
| Colon ca.* SW620(SW480 met) | 1.4 |
| Colon ca. HT29 | 0.0 |
| Colon ca. HCT-116 | 0.0 |
| Colon ca. CaCo-2 | 0.0 |
| Colon ca. tissue(ODO3866) | 0.0 |
| Colon ca. HCC-2998 | 4.8 |
| Gastric ca.* (liver met) NCI-N87 | 0.2 |
| Bladder | 2.6 |
| Trachea | 4.1 |
| Kidney | 0.6 |
| Kidney (fetal) | 1.3 |
| Renal ca. 786-0 | 31.2 |
| Renal ca. A498 | 4.4 |
| Renal ca. RXF 393 | 0.0 |
| Renal ca. ACHN | 1.7 |
| Renal ca. UO-31 | 4.2 |
| Renal ca. TK-10 | 5.8 |
| Liver | 0.0 |
| Liver (fetal) | 0.0 |
| Liver ca. (hepatoblast) HepG2 | 0.0 |
| Lung | 0.3 |
| Lung (fetal) | 2.1 |
| Lung ca. (small cell) LX-1 | 2.1 |
| Lung ca. (small cell) NCI-H69 | 4.8 |
| Lung ca. (s. cell var.) SHP-77 | 30.6 |
| Lung ca. (large cell)NCI-H460 | 21.2 |
| Lung ca. (non-sm. cell) A549 | 2.3 |
| Lung ca. (non-s. cell) NCI-H23 | 36.1 |
| Lung ca. (non-s. cell) HOP-62 | 10.8 |
| Lung ca. (non-s. cl) NCI-H522 | 0.3 |
| Lung ca. (squam.) SW 900 | 3.2 |
| Lung ca. (squam.) NCI-H596 | 23.2 |
| Mammary gland | 1.5 |
| Breast ca.* (pl. ef) MCF-7 | 0.0 |
| Breast ca.* (pl. ef) MDA-MB-231 | 0.0 |
| Breast ca.* (pl. ef) T47D | 4.9 |
| Breast ca. BT-549 | 14.2 |
| Breast ca. MDA-N | 1.4 |
| Ovary | 3.6 |
| Ovarian ca. OVCAR-3 | 12.9 |
| Ovarian ca. OVCAR-4 | 4.1 |
| Ovarian ca. OVCAR-5 | 0.0 |
| Ovarian ca. OVCAR-8 | 11.7 |
| Ovarian ca. IGROV-1 | 0.0 |
| Ovarian ca.* (ascites) SK-OV-3 | 1.6 |
| Uterus | 0.0 |
| Placenta | 0.0 |
| Prostate | 1.2 |
| Prostate ca.* (bone met)PC-3 | 0.6 |
| Testis | 0.8 |
| Melanoma Hs688(A).T | 0.0 |
| Melanoma* (met) Hs688(B).T | 0.0 |
| Melanoma UACC-62 | 1.1 |
| Melanoma M14 | 2.7 |
| Melanoma LOX IMVI | 0.8 |
| Melanoma* (met) SK-MEL-5 | 2.3 |
| Adipose | 1.3 |

Column A - Rel. Exp. (%) Ag2502, Run 165518160

TABLE BEC
Panel 2D

| Tissue Name | A | B |
|---|---|---|
| Normal Colon | 7.1 | 9.9 |
| CC Well to Mod Diff (ODO3866) | 0.0 | 0.1 |
| CC Margin (ODO3866) | 0.7 | 0.3 |
| CC Gr.2 rectosigmoid (ODO3868) | 0.3 | 0.0 |
| CC Margin (ODO3868) | 0.4 | 0.8 |
| CC Mod Diff (ODO3920) | 10.5 | 11.1 |
| CC Margin (ODO3920) | 1.0 | 1.5 |
| CC Gr.2 ascend colon (ODO3921) | 2.8 | 1.3 |
| CC Margin (ODO3921) | 0.1 | 0.2 |
| CC from Partial Hepatectomy | 0.0 | 0.0 |
| (ODO4309) Mets | | |
| Liver Margin (ODO4309) | 0.1 | 0.1 |
| Colon mets to lung (OD04451-01) | 0.9 | 1.3 |
| Lung Margin (OD04451-02) | 0.9 | 0.4 |
| Normal Prostate 6546-1 | 13.8 | 7.2 |
| Prostate Cancer (OD04410) | 19.1 | 15.9 |
| Prostate Margin (OD04410) | 7.0 | 8.5 |
| Prostate Cancer (OD04720-01) | 33.2 | 34.9 |
| Prostate Margin (OD04720-02) | 43.5 | 56.6 |
| Normal Lung 061010 | 2.8 | 3.0 |
| Lung Met to Muscle (ODO4286) | 15.8 | 14.3 |
| Muscle Margin (ODO4286) | 0.5 | 0.6 |
| Lung Malignant Cancer (OD03126) | 0.9 | 0.7 |
| Lung Margin (OD03126) | 1.2 | 1.9 |
| Lung Cancer (OD04404) | 0.3 | 0.3 |
| Lung Margin (OD04404) | 0.4 | 0.5 |
| Lung Cancer (OD04565) | 0.1 | 0.5 |
| Lung Margin (OD04565) | 0.3 | 1.0 |
| Lung Cancer (OD04237-01) | 1.7 | 1.7 |
| Lung Margin (OD04237-02) | 0.1 | 0.0 |
| Ocular Mel Met to Liver (ODO4310) | 0.0 | 0.0 |
| Liver Margin (ODO4310) | 0.2 | 0.0 |
| Melanoma Mets to Lung (OD04321) | 0.0 | 0.1 |
| Lung Margin (OD04321) | 0.4 | 0.6 |
| Normal Kidney | 2.3 | 1.9 |
| Kidney Ca, Nuclear grade 2 (OD04338) | 2.8 | 2.9 |
| Kidney Margin (OD04338) | 0.4 | 0.3 |
| Kidney Ca Nuclear grade 1/2 (OD04339) | 0.4 | 0.3 |
| Kidney Margin (OD04339) | 0.4 | 0.9 |
| Kidney Ca, Clear cell type (OD04340) | 27.4 | 33.2 |
| Kidney Margin (OD04340) | 0.2 | 0.3 |
| Kidney Ca, Nuclear grade 3 (OD04348) | 3.0 | 3.0 |
| Kidney Margin (OD04348) | 0.4 | 0.0 |
| Kidney Cancer (OD04622-01) | 100.0 | 100.0 |
| Kidney Margin (OD04622-03) | 0.0 | 0.5 |
| Kidney Cancer (OD04450-01) | 0.1 | 0.8 |
| Kidney Margin (OD04450-03) | 0.8 | 0.7 |
| Kidney Cancer 8120607 | 4.7 | 3.7 |
| Kidney Margin 8120608 | 0.1 | 0.1 |
| Kidney Cancer 8120613 | 0.0 | 0.1 |
| Kidney Margin 8120614 | 0.2 | 0.6 |
| Kidney Cancer 9010320 | 1.4 | 2.1 |
| Kidney Margin 9010321 | 0.6 | 0.2 |
| Normal Uterus | 0.1 | 0.1 |
| Uterus Cancer 064011 | 0.3 | 0.7 |
| Normal Thyroid | 14.5 | 13.5 |
| Thyroid Cancer 064010 | 0.4 | 0.3 |
| Thyroid Cancer A302152 | 0.2 | 0.6 |
| Thyroid Margin A302153 | 14.9 | 12.4 |
| Normal Breast | 1.7 | 2.6 |
| Breast Cancer (OD04566) | 0.1 | 0.2 |
| Breast Cancer (OD04590-01) | 5.5 | 5.1 |
| Breast Cancer Mets (OD04590-03) | 4.2 | 2.6 |
| Breast Cancer Metastasis (OD04655-05) | 1.0 | 0.9 |
| Breast Cancer 064006 | 1.3 | 0.4 |
| Breast Cancer 1024 | 2.1 | 1.1 |
| Breast Cancer 9100266 | 0.1 | 0.1 |
| Breast Margin 9100265 | 2.5 | 2.5 |
| Breast Cancer A209073 | 2.4 | 0.6 |
| Breast Margin A209073 | 3.7 | 3.3 |
| Normal Liver | 0.0 | 0.1 |
| Liver Cancer 064003 | 0.6 | 1.2 |
| Liver Cancer 1025 | 0.0 | 0.0 |
| Liver Cancer 1026 | 0.0 | 0.1 |
| Liver Cancer 6004-T | 0.0 | 0.0 |
| Liver Tissue 6004-N | 0.3 | 0.2 |
| Liver Cancer 6005-T | 0.1 | 0.0 |
| Liver Tissue 6005-N | 0.0 | 0.0 |
| Normal Bladder | 6.5 | 4.0 |
| Bladder Cancer 1023 | 0.0 | 0.3 |
| Bladder Cancer A302173 | 24.0 | 23.5 |
| Bladder Cancer (OD04718-01) | 0.0 | 0.0 |
| Bladder Normal Adjacent (OD04718-03) | 0.1 | 0.1 |
| Normal Ovary | 2.1 | 3.9 |
| Ovarian Cancer 064008 | 4.9 | 6.3 |
| Ovarian Cancer (OD04768-07) | 10.4 | 7.8 |
| Ovary Margin (OD04768-08) | 0.4 | 0.2 |
| Normal Stomach | 6.2 | 5.2 |
| Gastric Cancer 9060358 | 3.1 | 7.5 |
| Stomach Margin 9060359 | 1.3 | 0.8 |
| Gastric Cancer 9060395 | 4.7 | 4.3 |
| Stomach Margin 9060394 | 1.5 | 1.3 |
| Gastric Cancer 9060397 | 1.4 | 1.2 |
| Stomach Margin 9060396 | 0.2 | 0.2 |
| Gastric Cancer 064005 | 4.7 | 3.0 |

Column A - Rel. Exp. (%) Ag2502, Run 162319639
Column B - Rel. Exp. (%) Ag2502, Run 164993363

TABLE BED
Panel 3D

| Tissue Name | A |
|---|---|
| Daoy- Medulloblastoma | 1.6 |
| TE671- Medulloblastoma | 41.8 |
| D283 Med- Medulloblastoma | 63.7 |
| PFSK-1- Primitive Neuroectodermal | 0.0 |
| XF-498- CNS | 22.8 |
| SNB-78- Glioma | 11.8 |
| SF-268- Glioblastoma | 5.1 |
| T98G- Glioblastoma | 0.0 |
| SK-N-SH- Neuroblastoma (metastasis) | 0.0 |
| SF-295- Glioblastoma | 0.8 |
| Cerebellum | 79.0 |
| Cerebellum | 26.4 |
| NCI-H292- Mucoepidermoid lung carcinoma | 1.5 |
| DMS-114- Small cell lung cancer | 13.2 |
| DMS-79- Small cell lung cancer | 0.0 |
| NCI-H146- Small cell lung cancer | 28.5 |
| NCI-H526- Small cell lung cancer | 3.1 |
| NCI-N417- Small cell lung cancer | 46.3 |
| NCI-H82- Small cell lung cancer | 87.1 |
| NCI-H157- Squamous cell lung cancer (metastasis) | 100.0 |
| NCI-H1155- Large cell lung cancer | 35.6 |
| NCI-H1299- Large cell lung cancer | 16.5 |
| NCI-H727- Lung carcinoid | 0.0 |
| NCI-UMC-11- Lung carcinoid | 53.2 |
| LX-1- Small cell lung cancer | 1.2 |
| Colo-205- Colon cancer | 5.9 |
| KM12- Colon cancer | 0.0 |
| KM20L2- Colon cancer | 0.0 |
| NCI-H716- Colon cancer | 0.0 |
| SW-48- Colon adenocarcinoma | 0.0 |
| SW1116- Colon adenocarcinoma | 0.0 |
| LS 174T- Colon adenocarcinoma | 0.0 |
| SW-948- Colon adenocarcinoma | 0.0 |
| SW-480- Colon adenocarcinoma | 0.0 |
| NCI-SNU-5- Gastric carcinoma | 0.0 |
| KATO III- Gastric carcinoma | 0.0 |
| NCI-SNU-16- Gastric carcinoma | 6.7 |
| NCI-SNU-1- Gastric carcinoma | 0.0 |
| RF-1- Gastric adenocarcinoma | 2.4 |
| RF-48- Gastric adenocarcinoma | 2.6 |
| MKN-45- Gastric carcinoma | 0.0 |
| NCI-N87- Gastric carcinoma | 0.0 |
| OVCAR-5- Ovarian carcinoma | 0.0 |
| RL95-2- Uterine carcinoma | 1.7 |
| HelaS3- Cervical adenocarcinoma | 14.6 |
| Ca Ski- Cervical epidermoid carcinoma (metastasis) | 1.2 |
| ES-2- Ovarian clear cell carcinoma | 22.5 |
| Ramos- Stimulated with PMA/ionomycin 6 h | 0.0 |
| Ramos- Stimulated with PMA/ionomycin 14 h | 0.0 |
| MEG-01- Chronic myelogenous leukemia | 13.8 |
| (megokaryoblast) | |
| Raji- Burkitt's lymphoma | 0.0 |
| Daudi- Burkitt's lymphoma | 0.0 |
| U266- B-cell plasmacytoma | 8.5 |
| CA46- Burkitt's lymphoma | 6.3 |
| RL- non-Hodgkin's B-cell lymphoma | 0.0 |
| JM1- pre-B-cell lymphoma | 0.0 |
| Jurkat- T cell leukemia | 0.0 |
| TF-1- Erythroleukemia | 11.8 |
| HUT 78- T-cell lymphoma | 0.0 |
| U937- Histiocytic lymphoma | 0.0 |
| KU-812- Myelogenous leukemia | 0.3 |
| 769-P- Clear cell renal carcinoma | 9.5 |
| Caki-2- Clear cell renal carcinoma | 3.1 |
| SW 839- Clear cell renal carcinoma | 12.5 |
| Rhabdoid kidney tumor | 12.6 |
| Hs766T- Pancreatic carcinoma (LN metastasis) | 0.0 |
| CAPAN-1- Pancreatic adenocarcinoma (liver | 0.0 |
| metastasis) | |
| SU86.86- Pancreatic carcinoma (liver metastasis) | 2.4 |
| BxPC-3- Pancreatic adenocarcinoma | 0.0 |
| HPAC- Pancreatic adenocarcinoma | 0.0 |
| MIA PaCa-2- Pancreatic carcinoma | 0.7 |
| CFPAC-1- Pancreatic ductal adenocarcinoma | 0.4 |
| PANC-1- Pancreatic epithelioid ductal carcinoma | 1.3 |
| T24- Bladder carcinma (transitional cell) | 0.0 |
| 5637- Bladder carcinoma | 7.7 |
| HT-1197- Bladder carcinoma | 0.0 |
| UM-UC-3- Bladder carcinma (transitional cell) | 3.1 |
| A204- Rhabdomyosarcoma | 0.0 |
| HT-1080- Fibrosarcoma | 2.6 |
| MG-63- Osteosarcoma | 4.6 |
| SK-LMS-1- Leiomyosarcoma (vulva) | 0.3 |
| SJRH30- Rhabdomyosarcoma (met to bone marrow) | 22.8 |
| A431- Epidermoid carcinoma | 0.0 |
| WM266-4- Melanoma | 15.8 |
| DU 145- Prostate carcinoma (brain metastasis) | 0.0 |
| MDA-MB-468- Breast adenocarcinoma | 0.3 |
| SCC-4- Squamous cell carcinoma of tongue | 0.0 |
| SCC-9- Squamous cell carcinoma of tongue | 0.0 |
| SCC-15- Squamous cell carcinoma of tongue | 0.0 |
| CAL 27- Squamous cell carcinoma of tongue | 0.0 |

Column A - Rel. Exp. (%) Ag2502, Run 164629448

TABLE BEE
Panel 4D

| Tissue Name | A |
|---|---|
| Secondary Th1 act | 1.6 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 1.8 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 3.1 |
| 2ry Th1/Th2/Tr1_anti-CD95 CH11 | 1.1 |
| LAK cells rest | 4.1 |
| LAK cells IL-2 | 6.6 |
| LAK cells IL-2 + IL-12 | 0.0 |
| LAK cells IL-2 + IFN gamma | 2.4 |
| LAK cells IL-2 + IL-18 | 4.4 |
| LAK cells PMA/ionomycin | 0.0 |
| NK Cells IL-2 rest | 2.4 |
| Two Way MLR 3 day | 0.0 |
| Two Way MLR 5 day | 0.0 |
| Two Way MLR 7 day | 0.0 |
| PBMC rest | 7.8 |
| PBMC PWM | 6.7 |
| PBMC PHA-L | 0.0 |
| Ramos (B cell) none | 6.6 |
| Ramos (B cell) ionomycin | 8.8 |
| B lymphocytes PWM | 0.0 |
| B lymphocytes CD40L and IL-4 | 0.0 |
| EOL-1 dbcAMP | 100.0 |
| EOL-1 dbcAMP PMA/ionomycin | 18.2 |
| Dendritic cells none | 0.0 |
| Dendritic cells LPS | 0.0 |
| Dendritic cells anti-CD40 | 0.0 |
| Monocytes rest | 3.5 |
| Monocytes LPS | 0.0 |
| Macrophages rest | 0.0 |
| Macrophages LPS | 0.0 |
| HUVEC none | 0.0 |
| HUVEC starved | 23.3 |
| HUVEC IL-1beta | 3.5 |
| HUVEC IFN gamma | 0.0 |
| HUVEC TNF alpha + IFN gamma | 0.0 |
| HUVEC TNF alpha + IL4 | 0.0 |
| HUVEC IL-11 | 3.7 |
| Lung Microvascular EC none | 2.8 |
| Lung Microvascular EC TNFalpha + IL-1beta | 0.0 |
| Microvascular Dermal EC none | 0.0 |
| Microsvasular Dermal EG TNFalpha + IL-1beta | 0.0 |
| Bronchial epithelium TNFalpha + IL1beta | 9.7 |
| Small airway epithelium none | 23.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 21.6 |
| Astrocytes TNFalpha + IL-1 beta | 4.1 |
| KU-812 (Basophil) rest | 0.0 |
| KU-812 (Basophil) PMA/ionomycin | 46.3 |
| CCD1106 (Keratinocytes) none | 2.5 |
| CCD1106 (Keratinocytes) TNFalpha + IL-1beta | 6.9 |
| Liver cirrhosis | 16.6 |
| Lupus kidney | 2.6 |
| NCI-H292 none | 57.8 |
| NCI-H292 IL-4 | 26.8 |
| NCI-H292 IL-9 | 54.3 |
| NCI-H292 IL-13 | 24.0 |
| NCI-H292 IFN gamma | 20.7 |
| HPAEC none | 0.0 |
| HPAEC TNF alpha + IL-1 beta | 0.0 |
| Lung fibroblast none | 5.8 |
| Lung fibroblast TNF alpha + IL-1 beta | 0.0 |
| Lung fibroblast IL-4 | 31.4 |
| Lung fibroblast IL-9 | 7.6 |
| Lung fibroblast IL-13 | 3.0 |
| Lung fibroblast IFN gamma | 5.3 |
| Dermal fibroblast CCD1070 rest | 9.5 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| IBD Colitis 2 | 6.0 |
| IBD Crohn's | 1.3 |
| Colon | 31.4 |
| Lung | 56.3 |
| Thymus | 25.3 |
| Kidney | 12.2 |

Column A - Rel. Exp. (%) Ag2502, Run 162293442

Panel 1.3D Summary: Ag2502 Highest expression of this gene was seen in the cerebral cortex (CT=27). In addition, low levels of expression were seen in all CNS regions examined in this panel. This gene encodes a leucine-rich repeat protein. Leucine rich repeats (LRR) mediate reversible protein-protein interactions and have diverse cellular functions, including cellular adhesion and signaling. Several of these proteins, such as connectin, slit, chaoptin, and Toll have pivotal roles in neuronal development in Drosophila and play significant but distinct roles in neural development and in the adult nervous system of humans (Battye R. J. Neurosci. 21: 4290-4298). In Drosophilia, the LRR region of axon guidance proteins are critical for their function , especially in axon repulsion. Since the leucine-rich-repeat protein encoded by this gene was highly expressed in the cerebral cortex, it is a candidate neuronal guidance protein for axons, dendrites and/or growth cones in general. (Itoh A. Brain Res. Mol. Brain Res. 62: 175-186.) Therapeutic modulation of the levels of this protein, or signaling via this protein, are of utility in enhancing/directing compensatory synaptogenesis and fiber growth in the CNS in response to neuronal death (stroke, head trauma), axon lesion (spinal cord injury), or neurodegeneration (Alzheimer's, Parkinson's, Huntington's, vascular dementia or any neurodegenerative disease).

Moderate levels of expression were also seen in cell lines derived from ovarian cancer, lung cancer, and brain cancer. Therapeutic modulation of the expression or function of this gene or gene product is an effective treatment of these cancers.

Among metabolically relevant tissues, this gene was expressed in fetal skeletal muscle, thyroid, and pituitary gland. Therapeutic modulation of this gene or gene product is useful in the treatment of metabolic diseases such as obesity and diabetes, as well as neuroendocrine disorders. Glycoprotein hormones influence the development and function of the ovary, testis and thyroid by binding to specific high-affinity receptors. (Jiang X. Structure 3: 1341-1353). The extracellular domains of these receptors are members of the leucine-rich repeat (LRR) protein superfamily and are responsible for the high-affinity binding.

Panel 2D Summary: Ag2502 Highest expression of this gene was seen in kidney cancer (CTs=27.7). In addition, expression was significantly higher in the kidney cancer samples when compared to expression in samples from the normal adjacent tissue. There was also moderate to low expression in bladder, gastric, colon and ovarian cancers. Expression of this gene is useful as a marker to differentiate the kidney cancer samples from other samples on this panel and as a marker for kidney cancer. Therapeutic targeting of this gene or its protein product with a human monoclonal antibody, small molecule, or protein therapeutic is useful for limiting or blocking the extent of tumor cell migration, invasion, and metastasis, specifically in kidney, ovarian, bladder, gastric, and colon tumors.

Panel 3D Summary: Ag2502 Highest expression of this gene was seen in a lung cancer cell line (CT=28). Moderate levels of expression were seen in a cluster of lung and brain cancer cell lines. Prominent expression was also seen in cerebellum, in agreement with expression seen in Panel 1.3D. Low, but significant expression was also seen in kidney cancer and ovarian cancer cell lines. Expression of this gene is useful as a marker to differentiate lung and brain cancer cell lines and normal brain from other samples on this panel and as a marker for lung and brain cancer. Moderate expression of this gene was also seen in melanoma, rhabdomyosarcoma, osteosarcoma, renal and bladder carcinoma, lymphoma, ovarian and cervical cancer and gastric cancer cell lines. Therapeutic modulation of the expression or function of this gene is useful for the treatment of these cancers.

Panel 4D Summary: Ag2502 Ag2502 Highest expression of this gene was seen in eosinophils (CT=32). Higher levels of gene expression were observed in the eosinophil cell line EOL-1 under resting conditions over the levels in EOL-1 cells stimulated by phorbol ester and ionomycin (CT=34.4). Thus, this gene is involved in eosinophil function. Antibodies raised against this protein that stimulate its activity are useful in the reduction of eosinophil activation and as therapeutic antibodies for the treatment of asthma and allergy and as an anti-inflammatory therapeutics for T cell-mediated autoimmune and inflammatory diseases. Low but significant levels of expression were also seen in a cluster of treated and untreated NCI-H292 mucoepidermoid cells adn in normal colon, lung and thymus. This pattern of restricted expression shows that this gene or gene product is involved in the normal homeostasis of these tissues and/or pathological/inflammatory conditions of the lung.

BF. CG92813-01: Cadherin-related Tumor Suppressor Precursor (FAT)

Expression of gene CG92813-01 was assessed using the primer-probe sets Ag1413, Ag1515, Ag3085, Ag740 and Ag3819, described in Tables BFA, BFB, BFC, BFD, and BFE. Results of the RTQ-PCR runs are shown in Tables BFF, BFG, BFH and BFI.

TABLE BFA
Probe Name Ag1413

| | | | | SEQ |
|---|---|---|---|---|
| | | | Start | ID |
| Primers | Sequences | Length | Position | No |
| Forward | 5′-aggattctggtgttcctcaaat-3′ | 22 | 10908 | 1558 |
| Probe | TET-5′-tcttccacaggaactgtgcatatcaca-3′- | 27 | 10931 | 1559 |
| | TAMRA | | | |
| Reverse | 5′-cgagactgtgaaggattgtcat-3′ | 22 | 10971 | 1560 |

TABLE BFB
Probe Name Ag1515

| | | | | SEQ |
|---|---|---|---|---|
| | | | Start | ID |
| Primers | Sequences | Length | Position | No |
| Forward | 5′-ggatggttccatatcagtgaac-3′ | 22 | 2078 | 1561 |
| Probe | TET-5′-ctcgtgaccactgggtcctctgg-3′-TAMRA | 23 | 2108 | 1562 |
| Reverse | 5′-agaacaatctgggaagcaagtt-3′ | 22 | 2145 | 1563 |

TABLE BFC
Probe Name Ag3085

| | | | | SEQ |
|---|---|---|---|---|
| | | | Start | ID |
| Primers | Sequences | Length | Position | No |
| Forward | 5′-cttcacctgtagctgccca-3′ | 19 | 13801 | 1564 |
| Probe | TET-5′-acacgggaaggacctgtgagatggt-3′- | 25 | 13827 | 1565 |
| | TAMRA | | | |
| Reverse | 5′-acagaggacgccaagacag-3′ | 19 | 13858 | 1566 |

TABLE BFD
Probe Name Ag740

| | | | | SEQ |
|---|---|---|---|---|
| | | | Start | ID |
| Primers | Sequences | Length | Position | No |
| Forward | 5′-gagggatattgtcagggtcatc-3′ | 22 | 14126 | 1567 |
| Probe | TET-5′-aaaagcaacgttctcacttccctttt-3′- | 26 | 14100 | 1568 |
| | TAMRA | | | |
| Reverse | 5′-aaatcccaaagaggagaagaaa-3′ | 22 | 14062 | 1569 |

TABLE BFE
Probe Name Ag3819

| | | | | SEQ |
|---|---|---|---|---|
| | | | Start | ID |
| Primers | Sequences | Length | Position | No |
| Forward | 5′-agtcatcaatggctcgcata-3′ | 20 | 2523 | 1570 |
| Probe | TET-5′-tcttctggatataaatgataacagccctg-3′- | 29 | 2551 | 1571 |
| | TAMRA | | | |
| Reverse | 5′-aagtattggaccgggtagaaga-3′ | 22 | 2580 | 1572 |

TABLE BFF
General_screening_panel_v1.4

| Tissue Name | A | B |
|---|---|---|
| Adipose | 11.3 | 5.8 |
| Melanoma* Hs688(A).T | 84.1 | 68.8 |
| Melanoma* Hs688(B).T | 29.7 | 20.7 |
| Melanoma* M14 | 0.0 | 0.0 |
| Melanoma* LOXIMVI | 17.7 | 14.6 |
| Melanoma* SK-MEL-5 | 0.7 | 1.2 |
| Squamous cell carcinoma SCC-4 | 2.2 | 2.2 |
| Testis Pool | 4.5 | 4.1 |
| Prostate ca.* (bone met) PC-3 | 5.0 | 3.5 |
| Prostate Pool | 10.6 | 7.7 |
| Placenta | 2.5 | 3.7 |
| Uterus Pool | 4.6 | 5.8 |
| Ovarian ca. OVCAR-3 | 3.6 | 2.9 |
| Ovarian ca. SK-OV-3 | 46.0 | 7.7 |
| Ovarian ca. OVCAR-4 | 10.4 | 7.9 |
| Ovarian ca. OVCAR-5 | 5.8 | 3.7 |
| Ovarian ca. IGROV-1 | 1.8 | 1.5 |
| Ovarian ca. OVCAR-8 | 3.7 | 3.3 |
| Ovary | 25.2 | 13.6 |
| Breast ca. MCF-7 | 0.4 | 0.2 |
| Breast ca. MDA-MB-231 | 54.3 | 40.1 |
| Breast ca. BT 549 | 32.5 | 28.7 |
| Breast ca. T47D | 7.2 | 5.6 |
| Breast ca. MDA-N | 0.1 | 0.1 |
| Breast Pool | 48.6 | 15.9 |
| Trachea | 8.8 | 11.3 |
| Lung | 6.0 | 5.9 |
| Fetal Lung | 70.7 | 59.5 |
| Lung ca. NCI-N417 | 42.9 | 0.3 |
| Lung ca. LX-1 | 0.0 | 0.0 |
| Lung ca. NCI-H146 | 0.5 | 0.2 |
| Lung ca. SHP-77 | 2.8 | 2.5 |
| Lung ca. A549 | 1.4 | 1.7 |
| Lung ca. NCI-H526 | 0.0 | 0.0 |
| Lung ca. NCI-H23 | 100.0 | 100.0 |
| Lung ca. NCI-H460 | 44.1 | 6.5 |
| Lung ca. HOP-62 | 29.9 | 20.6 |
| Lung ca. NCI-H522 | 1.1 | 1.6 |
| Liver | 1.4 | 0.6 |
| Fetal Liver | 16.7 | 13.5 |
| Liver ca. HepG2 | 0.4 | 0.6 |
| Kidney Pool | 35.8 | 24.7 |
| Fetal Kidney | 48.6 | 25.7 |
| Renal ca. 786-0 | 37.6 | 30.8 |
| Renal ca. A498 | 7.6 | 6.0 |
| Renal ca. ACHN | 5.6 | 3.4 |
| Renal ca. UO-31 | 37.4 | 22.7 |
| Renal ca. TK-10 | 6.4 | 4.6 |
| Bladder | 6.4 | 4.5 |
| Gastric ca. (liver met.) NCI-N87 | 0.9 | 0.5 |
| Gastric ca. KATO III | 0.0 | 0.0 |
| Colon ca. SW-948 | 0.0 | 0.0 |
| Colon ca. SW480 | 0.0 | 0.0 |
| Colon ca.* (SW480 met) SW620 | 0.0 | 0.0 |
| Colon ca. HT29 | 0.0 | 0.0 |
| Colon ca. HCT-116 | 1.3 | 1.2 |
| Colon ca. CaCo-2 | 0.1 | 0.0 |
| Colon cancer tissue | 7.4 | 4.0 |
| Colon ca. SW1116 | 0.0 | 0.0 |
| Colon ca. Colo-205 | 42.3 | 0.0 |
| Colon ca. SW-48 | 0.0 | 0.0 |
| Colon Pool | 14.9 | 11.9 |
| Small Intestine Pool | 17.8 | 16.6 |
| Stomach Pool | 48.0 | 15.0 |
| Bone Marrow Pool | 6.3 | 7.2 |
| Fetal Heart | 12.7 | 8.0 |
| Heart Pool | 7.9 | 6.8 |
| Lymph Node Pool | 21.6 | 19.1 |
| Fetal Skeletal Muscle | 9.7 | 11.2 |
| Skeletal Muscle Pool | 5.0 | 3.9 |
| Spleen Pool | 14.8 | 16.3 |
| Thymus Pool | 16.3 | 16.2 |
| CMS cancer (glio/astro) U87-MG | 6.4 | 6.1 |
| CNS cancer (glio/astro) U-118-MG | 48.0 | 14.8 |
| CNS cancer (neuro; met) SK-N-AS | 50.0 | 28.3 |
| CNS cancer (astro) SF-539 | 22.7 | 16.5 |
| CNS cancer (astro) SNB-75 | 38.7 | 38.7 |
| CNS cancer (glio) SNB-19 | 2.7 | 2.4 |
| CNS cancer (glio) SF-295 | 27.9 | 16.8 |
| Brain (Amygdala) Pool | 7.1 | 4.1 |
| Brain (cerebellum) | 0.7 | 0.5 |
| Brain (fetal) | 75.3 | 52.5 |
| Brain (Hippocampus) Pool | 7.9 | 6.2 |
| Cerebral Cortex Pool | 4.7 | 4.2 |
| Brain (Substantia nigra) Pool | 4.4 | 3.2 |
| Brain (Thalamus) Pool | 9.0 | 5.4 |
| Brain (whole) | 44.4 | 4.1 |
| Spinal Cord Pool | 6.8 | 4.5 |
| Adrenal Gland | 2.3 | 2.1 |
| Pituitary gland Pool | 1.2 | 0.9 |
| Salivary Gland | 0.9 | 0.8 |
| Thyroid (female) | 7.8 | 4.7 |
| Pancreatic ca. CAPAN2 | 5.3 | 4.5 |
| Pancreas Pool | 20.2 | 14.0 |

Column A - Rel. Exp. (%) Ag1413, Run 213323517
Column B - Rel. Exp. (%) Ag3819, Run 218713598

TABLE BFG
Panel 2D

| Tissue Name | A | B |
|---|---|---|
| Normal Colon | 68.3 | 100.0 |
| CC Well to Mod Diff (ODO3866) | 6.3 | 5.0 |
| CC Margin (ODO3866) | 7.9 | 25.7 |
| CC Gr.2 rectosigmoid (ODO3868) | 4.6 | 12.6 |
| CC Margin (ODO3868) | 11.0 | 38.2 |
| CC Mod Diff (ODO3920) | 1.4 | 9.7 |
| CC Margin (ODO3920) | 12.1 | 54.3 |
| CC Gr.2 ascend colon (ODO3921) | 20.3 | 61.6 |
| CC Margin (ODO3921) | 10.9 | 12.9 |
| CC from Partial Hepatectomy (ODO4309) Mets | 4.9 | 3.3 |
| Liver Margin (ODO4309) | 10.4 | 9.0 |
| Colon mets to lung (OD04451-01) | 2.9 | 1.4 |
| Lung Margin (OD04451-02) | 10.2 | 11.5 |
| Normal Prostate 6546-1 | 2.7 | 5.1 |
| Prostate Cancer (OD04410) | 18.4 | 12.2 |
| Prostate Margin (OD04410) | 36.1 | 44.4 |
| Prostate Cancer (OD04720-01) | 17.3 | 28.9 |
| Prostate Margin (OD04720-02) | 28.5 | 39.5 |
| Normal Lung 061010 | 48.0 | 38.4 |
| Lung Met to Muscle (ODO4286) | 3.6 | 3.0 |
| Muscle Margin (ODO4286) | 5.9 | 3.5 |
| Lung Malignant Cancer (OD03126) | 11.7 | 9.7 |
| Lung Margin (OD03126) | 40.3 | 24.7 |
| Lung Cancer (OD04404) | 18.0 | 2.7 |
| Lung Margin (OD04404) | 32.8 | 9.0 |
| Lung Cancer (OD04565) | 2.9 | 2.7 |
| Lung Margin (OD04565) | 8.0 | 10.2 |
| Lung Cancer (OD04237-01) | 4.5 | 4.0 |
| Lung Margin (OD04237-02) | 31.4 | 17.9 |
| Ocular Mel Met to Liver (ODO4310) | 0.9 | 0.3 |
| Liver Margin (ODO4310) | 8.1 | 10.8 |
| Melanoma Mets to Lung (OD04321) | 2.8 | 1.5 |
| Lung Margin (OD04321) | 33.9 | 19.2 |
| Normal Kidney | 100.0 | 28.5 |
| Kidney Ca, Nuclear grade 2 (OD04338) | 14.1 | 9.8 |
| Kidney Margin (OD04338) | 24.0 | 7.1 |
| Kidney Ca Nuclear grade 1/2 (OD04339) | 14.3 | 6.7 |
| Kidney Margin (OD04339) | 22.8 | 4.5 |
| Kidney Ca, Clear cell type (OD04340) | 53.2 | 16.0 |
| Kidney Margin (OD04340) | 26.6 | 10.5 |
| Kidney Ca, Nuclear grade 3 (OD04348) | 8.9 | 3.6 |
| Kidney Margin (OD04348) | 14.1 | 10.0 |
| Kidney Cancer (OD04622-01) | 27.4 | 27.4 |
| Kidney Margin (OD04622-03) | 3.5 | 4.4 |
| Kidney Cancer (OD04450-01) | 8.4 | 2.6 |
| Kidney Margin (OD04450-03) | 23.8 | 7.5 |
| Kidney Cancer 8120607 | 1.3 | 0.4 |
| Kidney Margin 8120608 | 2.9 | 1.0 |
| Kidney Cancer 8120613 | 0.2 | 0.5 |
| Kidney Margin 8120614 | 6.3 | 2.0 |
| Kidney Cancer 9010320 | 23.0 | 2.4 |
| Kidney Margin 9010321 | 11.7 | 1.7 |
| Normal Uterus | 13.8 | 4.6 |
| Uterus Cancer 064011 | 17.8 | 9.3 |
| Normal Thyroid | 13.6 | 10.4 |
| Thyroid Cancer 064010 | 12.9 | 6.4 |
| Thyroid Cancer A302152 | 8.1 | 5.4 |
| Thyroid Margin A302153 | 29.1 | 11.0 |
| Normal Breast | 21.0 | 13.5 |
| Breast Cancer (OD04566) | 1.8 | 1.2 |
| Breast Cancer (OD04590-01) | 17.3 | 1.1 |
| Breast Cancer Mets (OD04590-03) | 23.3 | 3.7 |
| Breast Cancer Metastasis (OD04655-05) | 4.2 | 0.8 |
| Breast Cancer 064006 | 5.2 | 3.9 |
| Breast Cancer 1024 | 12.8 | 1.8 |
| Breast Cancer 9100266 | 3.9 | 1.7 |
| Breast Margin 9100265 | 10.2 | 5.0 |
| Breast Cancer A209073 | 12.2 | 10.1 |
| Breast Margin A209073 | 11.7 | 9.0 |
| Normal Liver | 7.1 | 4.4 |
| Liver Cancer 064003 | 10.3 | 8.0 |
| Liver Cancer 1025 | 5.0 | 5.1 |
| Liver Cancer 1026 | 3.0 | 1.5 |
| Liver Cancer 6004-T | 5.4 | 0.7 |
| Liver Tissue 6004-N | 1.4 | 0.3 |
| Liver Cancer 6005-T | 5.2 | 0.5 |
| Liver Tissue 6005-N | 3.3 | 3.5 |
| Normal Bladder | 7.1 | 7.7 |
| Bladder Cancer 1023 | 1.0 | 4.1 |
| Bladder Cancer A302173 | 3.0 | 3.6 |
| Bladder Cancer (OD04718-01) | 6.0 | 4.4 |
| Bladder Normal Adjacent (OD04718-03) | 27.5 | 25.2 |
| Normal Ovary | 6.7 | 4.8 |
| Ovarian Cancer 064008 | 27.0 | 21.9 |
| Ovarian Cancer (OD04768-07) | 0.7 | 0.2 |
| Ovary Margin (OD04768-08) | 12.4 | 6.7 |
| Normal Stomach | 27.4 | 12.3 |
| Gastric Cancer 9060358 | 6.9 | 2.1 |
| Stomach Margin 9060359 | 7.2 | 10.9 |
| Gastric Cancer 9060395 | 29.9 | 21.0 |
| Stomach Margin 9060394 | 13.7 | 16.3 |
| Gastric Cancer 9060397 | 23.2 | 18.3 |
| Stomach Margin 9060396 | 3.7 | 2.6 |
| Gastric Cancer 064005 | 18.3 | 26.8 |

Column A - Rel. Exp. (%) Ag1413, Run 169477489
Column B - Rel. Exp. (%) Ag740, Run 169590466

TABLE BFH
Panel 4.1D

| Tissue Name | A | B | C |
|---|---|---|---|
| Secondary Th1 act | 0.0 | 0.1 | 0.0 |
| Secondary Th2 act | 0.0 | 0.0 | 0.0 |
| Secondary Tr1 act | 0.0 | 0.1 | 0.0 |
| Secondary Th1 rest | 0.0 | 0.1 | 0.0 |
| Secondary Th2 rest | 0.2 | 0.0 | 0.0 |
| Secondary Tr1 rest | 0.0 | 0.1 | 0.0 |
| Primary Th1 act | 0.0 | 0.0 | 0.0 |
| Primary Th2 act | 0.0 | 0.0 | 0.0 |
| Primary Tr1 act | 0.0 | 0.0 | 0.0 |
| Primary Th1 rest | 0.0 | 0.0 | 0.0 |
| Primary Th2 rest | 0.0 | 0.0 | 0.0 |
| Primary Tr1 rest | 0.0 | 0.0 | 0.0 |
| CD45RA CD4 lymphocyte act | 3.4 | 4.1 | 3.0 |
| CD45RO CD4 lymphocyte act | 0.0 | 0.0 | 0.0 |
| CD8 lymphocyte act | 0.0 | 0.0 | 0.0 |
| Secondary CD8 lymphocyte rest | 0.0 | 0.0 | 0.0 |
| Secondary CD8 lymphocyte act | 0.0 | 0.0 | 0.0 |
| CD4 lymphocyte none | 0.2 | 0.1 | 0.0 |
| 2ry Th1/Th2/TM anti-CD95 CH11 | 0.1 | 0.0 | 0.0 |
| LAK cells rest | 0.3 | 0.0 | 0.0 |
| LAK cells IL-2 | 0.5 | 0.5 | 0.0 |
| LAK cells IL-2 + IL-12 | 0.0 | 0.1 | 0.0 |
| LAK cells IL-2 + IFN gamma | 0.1 | 0.3 | 0.0 |
| LAK cells IL-2 + IL-18 | 0.5 | 0.2 | 0.0 |
| LAK cells PMA/ionomycin | 0.1 | 0.1 | 0.0 |
| NK Cells IL-2 rest | 3.3 | 2.4 | 2.2 |
| Two Way MLR 3 day | 0.7 | 0.3 | 0.0 |
| Two Way MLR 5 day | 0.0 | 0.1 | 0.0 |
| Two Way MLR 7 day | 0.0 | 0.0 | 0.0 |
| PBMC rest | 0.2 | 0.2 | 0.0 |
| PBMC PWM | 0.1 | 0.0 | 0.0 |
| PBMC PHA-L | 0.1 | 0.0 | 0.0 |
| Ramos (B cell) none | 0.0 | 0.0 | 0.0 |
| Ramos (B cell) ionomycin | 0.0 | 0.0 | 0.0 |
| B lymphocytes PWM | 0.0 | 0.0 | 0.0 |
| B lymphocytes CD40L and IL-4 | 0.0 | 0.2 | 0.0 |
| EOL-1 dbcAMP | 0.0 | 0.0 | 0.0 |
| EOL-1 dbcAMP PMA/ionomycin | 0.0 | 0.0 | 0.0 |
| Dendritic cells none | 0.0 | 0.0 | 0.0 |
| Dendritic cells LPS | 0.0 | 0.0 | 0.0 |
| Dendritic cells anti-CD40 | 0.2 | 0.0 | 0.0 |
| Monocytes rest | 0.0 | 0.0 | 0.0 |
| Monocytes LPS | 0.0 | 0.0 | 0.0 |
| Macrophages rest | 0.0 | 0.0 | 0.0 |
| Macrophages LPS | 0.0 | 0.0 | 0.0 |
| HUVEC none | 16.2 | 17.9 | 19.5 |
| HUVEC starved | 37.1 | 38.7 | 49.0 |
| HUVEC IL-1beta | 21.3 | 24.5 | 21.2 |
| HUVEC IFN gamma | 100.0 | 100.0 | 100.0 |
| HUVEC TNF alpha + IFN gamma | 10.0 | 19.2 | 11.8 |
| HUVEC TNF alpha + IL4 | 4.0 | 5.2 | 3.1 |
| HUVEC IL-11 | 22.5 | 29.5 | 28.1 |
| Lung Microvascular EC none | 48.6 | 91.4 | 59.5 |
| Lung Microvascular EC | 12.5 | 18.8 | 8.6 |
| TNFalpha + IL-1beta | | | |
| Microvascular Dermal EC none | 62.9 | 58.6 | 62.4 |
| Microsvasular Dermal EC | 8.8 | 9.9 | 9.7 |
| TNFalpha + IL-1beta | | | |
| Bronchial epithelium TNFalpha + | 3.8 | 4.2 | 2.7 |
| IL1beta | | | |
| Small airway epithelium none | 1.8 | 2.1 | 1.2 |
| Small airway epithelium | 3.4 | 1.9 | 2.5 |
| TNFalpha + IL-1beta | | | |
| Coronery artery SMC rest | 4.2 | 3.4 | 4.4 |
| Coronery artery SMC TNFalpha + | 2.0 | 3.3 | 4.1 |
| IL-1beta | | | |
| Astrocytes rest | 7.0 | 6.3 | 6.0 |
| Astrocytes TNFalpha + IL-1beta | 3.7 | 4.2 | 6.5 |
| KU-812 (Basophil) rest | 1.1 | 1.5 | 2.0 |
| KU-812 (Basophil) PMA/ionomycin | 4.2 | 3.9 | 4.9 |
| CCD1106 (Keratinocytes) none | 1.0 | 3.0 | 1.7 |
| CCD1106 (Keratinocytes) | 1.6 | 1.3 | 0.0 |
| TNFalpha + IL-1beta | | | |
| Liver cirrhosis | 4.5 | 7.0 | 6.4 |
| NCI-H292 none | 0.3 | 0.9 | 1.1 |
| NCI-H292 IL-4 | 0.9 | 1.3 | 2.3 |
| NCI-H292 IL-9 | 2.0 | 2.9 | 2.8 |
| NCI-H292 IL-13 | 1.1 | 1.0 | 1.5 |
| NCI-H292 IFN gamma | 2.6 | 2.3 | 4.2 |
| HPAEC none | 27.0 | 41.5 | 52.9 |
| HPAEC TNF alpha + IL-1 beta | 6.0 | 7.0 | 4.0 |
| Lung fibroblast none | 10.2 | 14.4 | 6.7 |
| Lung fibroblast TNF alpha + IL-1 | 11.6 | 10.8 | 8.9 |
| beta | | | |
| Lung fibroblast IL-4 | 9.1 | 13.9 | 4.2 |
| Lung fibroblast IL-9 | 17.3 | 26.4 | 12.2 |
| Lung fibroblast IL-13 | 6.3 | 13.2 | 5.4 |
| Lung fibroblast IFN gamma | 11.2 | 9.4 | 6.6 |
| Dermal fibroblast CCD1070 rest | 11.1 | 15.9 | 23.2 |
| Dermal fibroblast CCD1070 TNF alpha | 4.6 | 6.3 | 6.3 |
| Dermal fibroblast CCD1070 IL-1 beta | 2.1 | 3.4 | 5.9 |
| Dermal fibroblast IFN gamma | 6.9 | 8.4 | 4.7 |
| Dermal fibroblast IL-4 | 15.5 | 17.2 | 21.8 |
| Dermal Fibroblasts rest | 17.6 | 14.0 | 12.8 |
| Neutrophils TNFa + LPS | 0.0 | 0.0 | 0.0 |
| Neutrophils rest | 0.0 | 0.0 | 0.0 |
| Colon | 6.0 | 5.2 | 5.5 |
| Lung | 17.1 | 15.1 | 25.3 |
| Thymus | 3.6 | 2.6 | 5.3 |
| Kidney | 11.4 | 12.1 | 14.9 |

Column A - Rel. Exp. (%) Ag1413, Run 169827815
Column B - Rel. Exp. (%) Ag3819, Run 170127253
Column C - Rel. Exp. (%) Ag740, Run 169827863

TABLE BFI
Panel 4D

| Tissue Name | A | B |
|---|---|---|
| Secondary Th1 act | 0.0 | 0.0 |
| Secondary Th2 act | 0.0 | 0.0 |
| Secondary Tr1 act | 0.0 | 0.2 |
| Secondary Th1 rest | 0.1 | 0.0 |
| Secondary Th2 rest | 0.0 | 0.0 |
| Secondary Tr1 rest | 0.0 | 0.2 |
| Primary Th1 act | 0.0 | 0.0 |
| Primary Th2 act | 0.0 | 0.0 |
| Primary Tr1 act | 0.0 | 0.0 |
| Primary Th1 rest | 0.0 | 0.0 |
| Primary Th2 rest | 0.0 | 0.0 |
| Primary Tr1 rest | 0.0 | 0.0 |
| CD45RA CD4 lymphocyte act | 3.8 | 4.6 |
| CD45RO CD4 lymphocyte act | 0.0 | 0.3 |
| CD8 lymphocyte act | 0.1 | 0.0 |
| Secondary CD8 lymphocyte rest | 0.0 | 0.0 |
| Secondary CD8 lymphocyte act | 0.0 | 0.0 |
| CD4 lymphocyte none | 0.0 | 0.0 |
| 2ry Th1/Th2/Tr1_anti-CD95 CH11 | 0.1 | 0.0 |
| LAK cells rest | 0.3 | 0.1 |
| LAK cells IL-2 | 0.5 | 0.4 |
| LAK cells IL-2 + IL-12 | 0.3 | 0.2 |
| LAK cells IL-2 + IFN gamma | 0.4 | 0.4 |
| LAK cells IL-2 + IL-18 | 0.5 | 0.0 |
| LAK cells PMA/ionomycin | 0.0 | 0.4 |
| NK Cells IL-2 rest | 2.2 | 1.8 |
| Two Way MLR 3 day | 0.4 | 0.5 |
| Two Way MLR 5 day | 0.1 | 0.1 |
| Two Way MLR 7 day | 0.1 | 0.0 |
| PBMC rest | 0.4 | 0.6 |
| PBMC PWM | 0.4 | 0.0 |
| PBMC PHA-L | 0.1 | 0.1 |
| Ramos (B cell) none | 0.0 | 0.0 |
| Ramos (B cell) ionomycin | 0.0 | 0.0 |
| B lymphocytes PWM | 0.0 | 0.0 |
| B lymphocytes CD40L and IL-4 | 0.1 | 0.0 |
| EOL-1 dbcAMP | 0.0 | 0.0 |
| EOL-1 dbcAMP PMA/ionomycin | 0.0 | 0.0 |
| Dendritic cells none | 0.0 | 0.0 |
| Dendritic cells LPS | 0.0 | 0.0 |
| Dendritic cells anti-CD40 | 0.0 | 0.0 |
| Monocytes rest | 0.0 | 0.0 |
| Monocytes LPS | 0.0 | 0.0 |
| Macrophages rest | 0.0 | 0.1 |
| Macrophages LPS | 0.0 | 0.0 |
| HUVEC none | 22.8 | 30.4 |
| HUVEC starved | 97.9 | 88.9 |
| HUVEC IL-1 beta | 17.8 | 8.5 |
| HUVEC IFN gamma | 100.0 | 100.0 |
| HUVEC TNF alpha + IFN gamma | 18.2 | 16.2 |
| HUVEC TNF alpha + IL4 | 5.4 | 4.2 |
| HUVEC IL-11 | 31.4 | 35.1 |
| Lung Microvascular EC none | 50.3 | 50.7 |
| Lung Microvascular EC TNFalpha + IL- | 9.0 | 6.8 |
| 1beta | | |
| Microvascular Dermal EC none | 71.7 | 83.5 |
| Microsvasular Dermal EC TNFalpha + IL- | 16.2 | 14.1 |
| 1beta | | |
| Bronchial epithelium TNFalpha + IL1beta | 6.7 | 5.6 |
| Small airway epithelium none | 2.0 | 2.1 |
| Small airway epithelium TNFalpha + IL- | 12.9 | 8.8 |
| 1beta | | |
| Coronery artery SMC rest | 5.4 | 4.8 |
| Coronery artery SMC TNFalpha + IL- | 2.2 | 2.6 |
| 1beta | | |
| Astrocytes rest | 5.1 | 8.9 |
| Astrocytes TNFalpha + IL-1beta | 5.8 | 5.8 |
| KU-812 (Basophil) rest | 1.2 | 0.7 |
| KU-812 (Basophil) PMA/ionomycin | 6.7 | 4.7 |
| CCD1106 (Keratinocytes) none | 2.0 | 4.4 |
| CCD1106 (Keratinocytes) TNFalpha + IL- | 1.1 | 0.7 |
| 1beta | | |
| Liver cirrhosis | 4.8 | 4.9 |
| Lupus kidney | 1.9 | 4.7 |
| NCI-H292 none | 2.1 | 1.5 |
| NCI-H292 IL-4 | 3.1 | 2.3 |
| NCI-H292 IL-9 | 3.7 | 3.1 |
| NCI-H292 IL-13 | 1.5 | 1.7 |
| NCI-H292 IFN gamma | 3.6 | 1.6 |
| HPAEC none | 29.3 | 40.1 |
| HPAEC TNF alpha + IL-1 beta | 4.5 | 5.3 |
| Lung fibroblast none | 12.7 | 19.1 |
| Lung fibroblast TNF alpha + IL-1 beta | 10.0 | 15.3 |
| Lung fibroblast IL-4 | 16.4 | 20.2 |
| Lung fibroblast IL-9 | 19.1 | 21.5 |
| Lung fibroblast IL-13 | 7.5 | 14.5 |
| Lung fibroblast IFN gamma | 17.2 | 19.6 |
| Dermal fibroblast CCD1070 rest | 30.6 | 27.0 |
| Dermal fibroblast CCD1070 TNF alpha | 8.4 | 10.1 |
| Dermal fibroblast CCD1070 IL-1 beta | 6.5 | 8.5 |
| Dermal fibroblast IFN gamma | 7.0 | 11.0 |
| Dermal fibroblast IL-4 | 17.6 | 15.7 |
| IBD Colitis 2 | 0.5 | 2.2 |
| IBD Crohn's | 1.8 | 3.0 |
| Colon | 14.6 | 15.6 |
| Lung | 17.9 | 21.0 |
| Thymus | 22.1 | 22.2 |
| Kidney | 6.7 | 5.2 |

Column A - Rel. Exp. (%) Ag1515, Run 163478545
Column B - Rel. Exp. (%) Ag3085, Run 164682194

General_screening_panel_v1.4 Summary: Ag1413/Ag3819 Highest expression of this gene was seen in a lung cancer cell line NCI-H23 sample (CT=26-28). High to moderate levels of expression of this gene were also seen in a cluster of CNS cancer, renal cancer, lung cancer, breast cancer, ovarian cancer and melanoma cell lines. Therefore, therapeutic modulation of the activity of this gene or its protein product, through the use of small molecule drugs, protein therapeutics or antibodies, is beneficial in the treatment of lung cancer or ovarian cancer. This gene encodes a cadherin-related tumor suppressor precursor. E-cadherin, a related protein is used as a prognostic marker for breast cancer detection (Barshack I, J Clin Pathol 54(9):684-8). Expression of this gene is useful as a diagnostic marker in the above mentioned cancers.

Among tissues with metabolic or endocrine function, this gene was expressed at high to moderate levels in pancreas, adipose, adrenal gland, thyroid, pituitary gland, skeletal muscle, heart, liver and the gastrointestinal tract. Therapeutic modulation of the activity of this gene is useful in the treatment of endocrine/metabolically related diseases, such as obesity and diabetes. In addition, E cadherin, a related protein is shown to be reduced in small intestinal mucosa of coeliac sprue disease (Barshak et. al), a sample not used in this panel. Therapeutic modulation of the activity or function of this gene or gene product is useful in the treatment of coelic sprue disease.

In addition, this gene was expressed at low to moderate levels in all regions of the central nervous system examined, including amygdala, hippocampus, substantia nigra, thalamus, cerebellum, cerebral cortex, and spinal cord. This gene product is a transmembrane glycoproteins belonging to the cadherin superfamily of molecules, which are involved in many biological processes such as cell adhesion, cytoskeletal organization and morphogenesis. Cadherins can act as axon guidance and cell adhesion proteins, specifically during development and in the response to injury (Ranscht B. Int. J. Dev. Neurosci. 18: 643-651.). Therefore, manipulation of levels of this protein are of use in inducing a compensatory synaptogenic response to neuronal death in Alzheimer's disease, Parkinson's disease, Huntington's disease, spinocerebellar ataxia, progressive supranuclear palsy, ALS, head trauma, stroke, or any other disease/condition associated with neuronal loss.

Panel 2D Summary: Ag1413/Ag740 Highest expression of this gene wass detected in samples from normal kidney and colon (CTs=29-30), with significant expression of this gene in both normal and cancer tissues. Expression of this gene was higher in control margin samples of colon (ODO3920), liver (OD04310), and ovary (OD04768-08) as compared to their corresponding cancer tissue. Please see Panel 1.4 for a discussion of the potential utility of this gene.

Panel 4.1D Summary: Ag1413/Ag3819/Ag740 Highest expression of this gene was detected in IFN gamma treated HUVEC cells (CTs=25-27). High to moderate expression of this gene was seen in treated and untreated HUVEC, lung microvascular EC, microvascular dermal EC, Bronchial epithelium, small airway epithelium, NCI-H292, HPAEC, lung fibroblasts, and dermal fibroblasts. The expression of this gene in cells derived from or within the lung shows that this gene is involved in normal conditions as well as pathological and inflammatory lung disorders that include chronic obstructive pulmonary disease, asthma, allergy and emphysema.

In addition, high expression of thisgene was also detected in normal tissues represented by colon, lung, thymus and kidney. Therapeutic modulation of the activity of the protein encoded by this gene is useful in the treatment of inflammatory disease affecting these tissues such as inflammatory bowel disease, chronic obstructive pulmonary disease, asthma, allergy, emphysema, lupus and glomerulonephritis.

Panel 4D Summary: Ag1515/Ag3085 Highest expression of this gene was detected in IFN gamma treated HUVEC cells (CT=25-27). In addition, high to moderate expression of this gene was seen in treated and untreated HVEC, lung microvascular EC, microvascular dermal EC, Bronchial epithelium, small airway epithelium, NCI-H292, HPAEC, lung fibroblasts, and dermal fibroblasts. The expression of this gene in cells derived from or within the lung shows that this gene is involved in normal conditions as well as pathological and inflammatory lung disorders that include chronic obstructive pulmonary disease, asthma, allergy and emphysema.

Expression of this gene was higher in untreated HPAEC (CTs=27-28) as compared to TNF alpha+IL-1 beta treated cells (CTs=30-31). Thus, expression of this gene is useful as a marker to distinguish the treated from untreated HIPAEC samples.

In addition, high levels of expression of this gene were also detected in normal tissues represented by colon, lung, thymus and kidney. Expression of this gene is lower in colon samples from patients with IBD colitis and Crohn's disease relative CTs=31-33) to normal colon (CTs=28-29). Therefore, therapeutic modulation of the activity or function of this gene or the protein encoded by this gene is useful in the treatment of inflammatory bowel disease.

BG. CG93387-05: Fibropellin I Precursor

Expression of gene CG93387-05 was assessed using the primer-probe sets Agl 143, Ag1921, and Ag3082, described in Tables BGA, BGB, and BGC. Results of the RTQ-PCR runs are shown in Tables BGD, BGE, and BGF.

TABLE BGA
Probe Name Ag1143

| | | | | |
|---|---|---|---|---|
| | | | | SEQ |
| | | | Start | ID |
| Primers | Sequences | Length | Position | No |

| | | | | |
|---|---|---|---|---|
| Forward | 5′-gagatggatgtggacgactg-3′ | 20 | 964 | 1573 |
| Probe | TET-5′-cctgaatggaggctcttgtgttgacc-3′- | 26 | 999 | 1574 |
| | TAMRA | | | |
| Reverse | 5′-acaagcaggtgtaattcccc-3′ | 20 | 1029 | 1575 |

TABLE BGB
Probe Name Ag1921

| | | | | SEQ |
|---|---|---|---|---|
| | | | Start | ID |
| Primers | Sequences | Length | Position | No |
| Forward | 5′-acacattccagactgtgctcat-3′ | 22 | 317 | 1576 |
| Probe | TET-5′-acggcaagctctccttcaccatctt-3′- | 25 | 344 | 1577 |
| | TAMRA | | | |
| Reverse | 5′-tccacacgatggactcatagtt-3′ | 22 | 370 | 1578 |

TABLE BGC
Probe Name Ag3082

| | | | | SEQ |
|---|---|---|---|---|
| | | | Start | ID |
| Primers | Sequences | Length | Position | No |
| Forward | 5′-cagacgcagaggtagctcc-3′ | 19 | 1385 | 1579 |
| Probe | TET-5′-catctgggcactgtgtgttcatgttg-3′- | 26 | 1335 | 1580 |
| | TAMRA | | | |
| Reverse | 5′-atttgaaatcacagccatgc-3′ | 20 | 1311 | 1581 |

TABLE BGD
Panel 1.3D

| Tissue Name | A |
|---|---|
| Liver adenocarcinoma | 31.2 |
| Pancreas | 2.5 |
| Pancreatic ca. CAPAN 2 | 0.2 |
| Adrenal gland | 12.3 |
| Thyroid | 19.5 |
| Salivary gland | 2.3 |
| Pituitary gland | 10.2 |
| Brain (fetal) | 2.1 |
| Brain (whole) | 10.8 |
| Brain (amygdala) | 12.3 |
| Brain (cerebellum) | 21.0 |
| Brain (hippocampus) | 21.8 |
| Brain (substantia nigra) | 3.9 |
| Brain (thalamus) | 8.7 |
| Cerebral Cortex | 8.7 |
| Spinal cord | 12.8 |
| glio/astro U87-MG | 3.0 |
| glio/astro U-118-MG | 100.0 |
| astrocytoma SW1783 | 6.9 |
| neuro*; met SK-N-AS | 3.1 |
| astrocytoma SF-539 | 12.6 |
| astrocytoma SNB-75 | 25.9 |
| glioma SNB-19 | 8.9 |
| glioma U251 | 4.9 |
| glioma SF-295 | 33.7 |
| Heart (Fetal) | 1.6 |
| Heart | 5.8 |
| Skeletal muscle (Fetal) | 5.8 |
| Skeletal muscle | 14.3 |
| Bone marrow | 1.0 |
| Thymus | 5.1 |
| Spleen | 22.5 |
| Lymph node | 28.5 |
| Colorectal | 3.6 |
| Stomach | 11.8 |
| Small intestine | 14.5 |
| Colon ca. SW480 | 0.8 |
| Colon ca.* SW620 (SW480 met) | 0.3 |
| Colon ca. HT29 | 0.0 |
| Colon ca. HCT-116 | 1.1 |
| Colon ca. CaCo-2 | 0.0 |
| CC Well to Mod Diff (ODO3866) | 3.1 |
| Colon ca. HCC-2998 | 0.4 |
| Gastric ca. (liver met) NCI-N87 | 3.5 |
| Bladder | 8.8 |
| Trachea | 7.5 |
| Kidney | 3.5 |
| Kidney (fetal) | 3.4 |
| Renal ca. 786-0 | 0.2 |
| Renal ca. A498 | 12.4 |
| Renal ca. RXF 393 | 0.0 |
| Renal ca. ACHN | 0.0 |
| Renal ca. UO-31 | 0.2 |
| Renal ca. TK-10 | 0.1 |
| Liver | 2.4 |
| Liver (fetal) | 1.6 |
| Liver ca. (hepatoblast) HepG2 | 0.0 |
| Lung | 9.0 |
| Lung (fetal) | 6.4 |
| Lung ca. (small cell) LX-1 | 2.9 |
| Lung ca. (small cell) NCI-H69 | 0.3 |
| Lung ca. (s. cell var.) SHP-77 | 4.0 |
| Lung ca. (large cell)NCI-H460 | 4.4 |
| Lung ca. (non-sm. cell) A549 | 1.3 |
| Lung ca. (non-s. cell) NCI-H23 | 2.9 |
| Lung ca. (non-s. cell) HOP-62 | 2.7 |
| Lung ca. (non-s. cl) NCI-H522 | 0.0 |
| Lung ca. (squam.) SW 900 | 6.0 |
| Lung ca. (squam.) NCI-H596 | 0.8 |
| Mammary gland | 12.5 |
| Breast ca.* (pl. ef) MCF-7 | 0.3 |
| Breast ca.* (pl. ef) MDA-MB-231 | 1.7 |
| Breast ca.* (pl. ef) T47D | 1.0 |
| Breast ca. BT-549 | 15.1 |
| Breast ca. MDA-N | 0.5 |
| Ovary | 13.4 |
| Ovarian ca. OVCAR-3 | 1.1 |
| Ovarian ca. OVCAR-4 | 0.0 |
| Ovarian ca. OVCAR-5 | 12.2 |
| Ovarian ca. OVCAR-8 | 0.5 |
| Ovarian ca. IGROV-1 | 0.0 |
| Ovarian ca. (ascites) SK-OV-3 | 7.2 |
| Uterus | 32.8 |
| Placenta | 1.1 |
| Prostate | 12.4 |
| Prostate ca.* (bone met) PC-3 | 0.0 |
| Testis | 8.1 |
| Melanoma Hs688(A).T | 2.6 |
| Melanoma* (met) Hs688(B).T | 0.9 |
| Melanoma UACC-62 | 4.8 |
| Melanoma M14 | 17.8 |
| Melanoma LOX IMVI | 0.2 |
| Melanoma* (met) SK-MEL-5 | 0.2 |
| Adipose | 17.2 |

Column A - Rel. Exp. (%) Ag3082, Run 165673172

TABLE BGE
Panel 2.2

| Tissue Name | A |
|---|---|
| Normal Colon | 20.3 |
| Colon cancer (OD06064) | 8.5 |
| Colon Margin (OD06064) | 6.4 |
| Colon cancer (OD06159) | 1.4 |
| Colon Margin (OD06159) | 9.6 |
| Colon cancer (OD06297-04) | 1.1 |
| Colon Margin (OD06297-05) | 5.7 |
| CC Gr.2 ascend colon (ODO3921) | 5.9 |
| CC Margin (ODO3921) | 2.9 |
| Colon cancer metastasis (OD06104) | 1.3 |
| Lung Margin (OD06104) | 0.8 |
| Colon mets to lung (OD04451-01) | 17.3 |
| Lung Margin (OD04451-02) | 17.2 |
| Normal Prostate | 20.3 |
| Prostate Cancer (OD04410) | 13.1 |
| Prostate Margin (OD04410) | 10.1 |
| Normal Ovary | 52.5 |
| Ovarian cancer (OD06283-03) | 8.6 |
| Ovarian Margin (OD06283-07) | 13.1 |
| Ovarian Cancer | 23.2 |
| Ovarian cancer (OD06145) | 6.4 |
| Ovarian Margin (OD06145) | 52.5 |
| Ovarian cancer (OD06455-03) | 13.6 |
| Ovarian Margin (OD06455-07) | 33.2 |
| Normal Lung | 19.6 |
| Invasive poor diff. lung adeno (ODO4945-01 | 7.7 |
| Lung Margin (ODO4945-03) | 48.3 |
| Lung Malignant Cancer (OD03126) | 11.0 |
| Lung Margin (OD03126) | 6.5 |
| Lung Cancer (OD05014A) | 11.8 |
| Lung Margin (OD05014B) | 49.0 |
| Lung cancer (OD06081) | 17.6 |
| Lung Margin (OD06081) | 37.9 |
| Lung Cancer (OD04237-01) | 1.2 |
| Lung Margin (OD04237-02) | 23.7 |
| Ocular Mel Met to Liver (ODO4310) | 44.1 |
| Liver Margin (ODO4310) | 14.9 |
| Melanoma Metastasis | 0.0 |
| Lung Margin (OD04321) | 16.3 |
| Normal Kidney | 9.8 |
| Kidney Ca, Nuclear grade 2 (OD04338) | 26.6 |
| Kidney Margin (OD04338) | 1.6 |
| Kidney Ca Nuclear grade 1/2 (OD04339) | 20.2 |
| Kidney Margin (OD04339) | 10.2 |
| Kidney Ca, Clear cell type (OD04340) | 20.9 |
| Kidney Margin (OD04340) | 13.3 |
| Kidney Ca, Nuclear grade 3 (OD04348) | 4.4 |
| Kidney Margin (OD04348) | 54.0 |
| Kidney malignant cancer (OD06204B) | 0.6 |
| Kidney normal adjacent tissue (OD06204E) | 8.5 |
| Kidney Cancer (OD04450-01) | 0.5 |
| Kidney Margin (OD04450-03) | 16.4 |
| Kidney Cancer 8120613 | 0.0 |
| Kidney Margin 8120614 | 11.0 |
| Kidney Cancer 9010320 | 1.5 |
| Kidney Margin 9010321 | 7.1 |
| Kidney Cancer 8120607 | 6.3 |
| Kidney Margin 8120608 | 5.9 |
| Normal Uterus | 52.1 |
| Uterine Cancer 064011 | 37.6 |
| Normal Thyroid | 10.2 |
| Thyroid Cancer | 4.0 |
| Thyroid Cancer A302152 | 21.0 |
| Thyroid Margin A302153 | 29.5 |
| Normal Breast | 52.1 |
| Breast Cancer | 2.7 |
| Breast Cancer | 43.5 |
| Breast Cancer (OD04590-01) | 59.5 |
| Breast Cancer Mets (OD04590-03) | 35.4 |
| Breast Cancer Metastasis | 44.1 |
| Breast Cancer | 17.9 |
| Breast Cancer 9100266 | 16.6 |
| Breast Margin 9100265 | 26.1 |
| Breast Cancer A209073 | 9.8 |
| Breast Margin A209073 | 28.3 |
| Breast cancer (OD06083) | 63.7 |
| Breast cancer node metastasis (OD06083) | 100.0 |
| Normal Liver | 39.8 |
| Liver Cancer 1026 | 17.2 |
| Liver Cancer 1025 | 40.3 |
| Liver Cancer 6004-T | 33.7 |
| Liver Tissue 6004-N | 2.2 |
| Liver Cancer 6005-T | 42.3 |
| Liver Tissue 6005-N | 44.8 |
| Liver Cancer | 4.2 |
| Normal Bladder | 17.6 |
| Bladder Cancer | 8.4 |
| Bladder Cancer | 3.2 |
| Normal Stomach | 25.2 |
| Gastric Cancer 9060397 | 0.7 |
| Stomach Margin 9060396 | 4.7 |
| Gastric Cancer 9060395 | 8.1 |
| Stomach Margin 9060394 | 12.2 |
| Gastric Cancer 064005 | 7.2 |

Column A - Rel. Exp. (%) Ag3082, Run 174284798

TABLE BGF
Panel 4D

| Tissue Name | A | B | C |
|---|---|---|---|
| Secondary Th1 act | 1.3 | 0.0 | 1.4 |
| Secondary Th2 act | 6.3 | 0.5 | 5.8 |
| Secondary Tr1 act | 5.5 | 0.7 | 3.8 |
| Secondary Th1 rest | 6.3 | 1.9 | 12.8 |
| Secondary Th2 rest | 17.0 | 2.0 | 19.2 |
| Secondary Tr1 rest | 12.9 | 0.8 | 18.8 |
| Primary Th1 act | 0.9 | 0.0 | 0.8 |
| Primary Th2 act | 0.7 | 0.0 | 2.3 |
| Primary Tr1 act | 1.5 | 0.1 | 2.3 |
| Primary Th1 rest | 21.0 | 5.8 | 50.7 |
| Primary Th2 rest | 32.5 | 7.5 | 0.0 |
| Primary Tr1 rest | 2.3 | 0.3 | 6.3 |
| CD45RA CD4 lymphocyte act | 2.5 | 1.4 | 2.3 |
| CD45RO CD4 lymphocyte act | 2.7 | 1.1 | 1.8 |
| CD8 lymphocyte act | 2.1 | 0.6 | 2.9 |
| Secondary CD8 | 2.9 | 0.5 | 4.2 |
| lymphocyte rest | | | |
| Secondary CD8 | 5.4 | 0.0 | 3.1 |
| lymphocyte act | | | |
| CD4 lymphocyte none | 11.7 | 3.7 | 10.8 |
| 2ry Th1/Th2/Tr1 anti- | 27.5 | 1.3 | 27.0 |
| CD95 CH11 | | | |
| LAK cells rest | 4.5 | 1.1 | 4.4 |
| LAK cells IL-2 | 6.8 | 3.7 | 11.3 |
| LAK cells IL-2 + IL-12 | 1.8 | 0.3 | 2.8 |
| LAK cells IL-2 + IFN | 3.2 | 1.1 | 6.4 |
| gamma | | | |
| LAK cells IL-2 + IL-18 | 1.7 | 0.5 | 7.6 |
| LAK cells PMA/ionomycin | 1.6 | 0.1 | 1.1 |
| NK Cells IL-2 rest | 2.2 | 0.8 | 3.2 |
| Two Way MLR 3 day | 5.6 | 2.3 | 6.0 |
| Two Way MLR 5 day | 1.1 | 0.4 | 1.4 |
| Two Way MLR 7 day | 0.9 | 0.5 | 1.4 |
| PBMC rest | 7.1 | 1.7 | 4.6 |
| PBMC PWM | 0.9 | 1.0 | 3.8 |
| PBMC PHA-L | 3.0 | 0.4 | 2.6 |
| Ramos (B cell) none | 0.0 | 0.0 | 0.0 |
| Ramos (B cell) ionomycin | 0.0 | 0.0 | 0.2 |
| B lymphocytes PWM | 5.3 | 2.0 | 22.8 |
| B lymphocytes CD40L | 18.0 | 3.6 | 57.8 |
| and IL-4 | | | |
| EOL-1 dbcAMP | 0.0 | 0.1 | 0.2 |
| EOL-1 dbcAMP PMA/ionomycin | 0.0 | 0.0 | 0.0 |
| Dendritic cells none | 3.3 | 4.2 | 5.1 |
| Dendritic cells LPS | 0.8 | 1.5 | 1.3 |
| Dendritic cells anti-CD40 | 4.3 | 4.6 | 5.4 |
| Monocytes rest | 2.4 | 8.6 | 6.3 |
| Monocytes LPS | 0.0 | 0.2 | 1.0 |
| Macrophages rest | 1.8 | 2.9 | 1.7 |
| Macrophages LPS | 0.7 | 0.5 | 0.5 |
| HUVEC none | 6.2 | 12.1 | 10.4 |
| HUVEC starved | 4.4 | 11.9 | 12.9 |
| HUVEC IL-1beta | 0.9 | 1.6 | 3.3 |
| HUVEC IFN gamma | 23.0 | 13.6 | 13.6 |
| HUVEC TNF alpha + IFN gamma | 2.1 | 3.5 | 7.5 |
| HUVEC TNF alpha + IL4 | 6.2 | 4.2 | 3.2 |
| HUVEC IL-11 | 12.1 | 8.7 | 8.1 |
| Lung Microvascular EC none | 27.7 | 24.3 | 21.5 |
| Lung Microvascular EC TNFalpha + | 8.7 | 14.5 | 16.6 |
| IL-1beta | | | |
| Microvascular Dermal EC none | 19.5 | 19.3 | 20.3 |
| Microsvasular Dermal EC | 10.1 | 11.3 | 10.3 |
| TNFalpha + IL-1beta | | | |
| Bronchial epithelium TNFalpha + | 0.3 | 0.4 | 1.0 |
| IL1beta | | | |
| Small airway epithelium none | 2.0 | 4.9 | 2.6 |
| Small airway epithelium TNFalpha + | 0.7 | 0.2 | 2.1 |
| IL-1beta | | | |
| Coronery artery SMC rest | 0.7 | 3.6 | 2.9 |
| Coronery artery SMC TNFalpha + | 6.7 | 1.4 | 3.7 |
| IL-1beta | | | |
| Astrocytes rest | 3.7 | 3.0 | 3.7 |
| Astrocytes TNFalpha + IL-1beta | 3.7 | 3.4 | 4.0 |
| KU-812 (Basophil) rest | 0.0 | 0.0 | 0.0 |
| KU-812 (Basophil) PMA/ionomycin | 0.0 | 0.1 | 0.1 |
| CCD1106 (Keratinocytes) none | 0.0 | 0.0 | 0.1 |
| 93580 CCD1106 (Keratinocytes) | 0.0 | 0.0 | 0.2 |
| TNFa and IFNg | | | |
| Liver cirrhosis | 3.5 | 4.5 | 5.9 |
| Lupus kidney | 15.0 | 6.1 | 10.2 |
| NCI-H292 none | 0.4 | 1.6 | 3.5 |
| NCI-H292 IL-4 | 0.6 | 1.3 | 3.4 |
| NCI-H292 IL-9 | 1.0 | 2.2 | 3.0 |
| NCI-H292 IL-13 | 3.5 | 0.4 | 0.8 |
| NCI-H292 IFN gamma | 2.3 | 1.0 | 1.3 |
| HPAEC none | 58.6 | 32.5 | 32.1 |
| HPAEC TNF alpha + IL-1 beta | 18.6 | 16.8 | 22.4 |
| Lung fibroblast none | 78.5 | 79.6 | 90.1 |
| Lung fibroblast TNF alpha + IL-1 | 20.0 | 18.0 | 24.0 |
| beta | | | |
| Lung fibroblast IL-4 | 48.3 | 100.0 | 100.0 |
| Lung fibroblast IL-9 | 35.4 | 56.6 | 49.7 |
| Lung fibroblast IL-13 | 100.0 | 64.2 | 77.4 |
| Lung fibroblast IFN gamma | 56.6 | 96.6 | 82.4 |
| Dermal fibroblast CCD1070 rest | 5.7 | 9.3 | 17.2 |
| Dermal fibroblast CCD1070 TNF | 11.3 | 6.0 | 25.5 |
| alpha | | | |
| Dermal fibroblast CCD1070 IL-1 | 20.3 | 4.0 | 6.5 |
| beta | | | |
| Dermal fibroblast IFN gamma | 30.8 | 9.2 | 18.7 |
| Dermal fibroblast IL-4 | 35.1 | 17.8 | 26.4 |
| IBD Colitis 2 | 4.1 | 2.8 | 5.5 |
| IBD Crohn's | 1.7 | 1.4 | 2.2 |
| Colon | 6.2 | 7.4 | 8.5 |
| Lung | 13.9 | 19.1 | 20.3 |
| Thymus | 25.2 | 16.4 | 14.7 |
| Kidney | 13.8 | 8.6 | 31.0 |

Column A - Rel. Exp. (%) Ag1143, Run 139943479
Column B - Rel. Exp. (%) Ag1921, Run 164629443
Column C - Rel. Exp. (%) Ag3082, Run 164681898

Panel 1.3D Summary: Ag3082 Highest expression of this gene was seen in a brain cancer cell line (CT=27.3). Significant levels of expression were also seen in a cluster of samples derived from ovarian, breast, melanoma and brain cancer cell lines. Thus, expression of this gene is useful as a marker to differentiate between the brain cancer samples and other samples on this panel and as a marker to detect the presence of these cancers. This gene encodes a protein that is homologous to an epidermal growth factor related protein (fibropellin like). Fibropellins are a family of extracellular sea urchin matrix proteins that have been implicated in cell adhestion. Therefore, therapeutic modulation of the expression or function of this gene is useful in the treatment of ovarian, breast, melanoma and brain cancers.

Among tissues with metabolic function, this gene was expressed at moderate to low levels in pituitary, adipose, adrenal gland, pancreas, thyroid, and adult and fetal skeletal muscle, heart, and liver. This widespread expression among these tissues shows that this gene product plays a role in normal neuroendocrine and metabolic and that disregulated expression of this gene contributes to neuroendocrine disorders or metabolic diseases, such as obesity and diabetes.

Moderate to low levels of expression were also seen in all regions of the CNS examined, including pituitary, amygdala, thalamus, substantia nigra, cerebral cortex, and hippocampus. Therefore, therapeutic modulation of the expression or function of this gene is useful in the treatment of neurologic disorders, such as Alzheimer's disease, Parkinson's disease, schizophrenia, multiple sclerosis, stroke and epilepsy.

Panel 2.2 Summary: Ag3082 Highest expression of this gene was seen in a breast cancer metastasis (CT=28.3). Significant levels of expression were also seen in a cluster of breast cancer samples. Conversely, expression was higher in normal ovary and lung tissue when compared to expression in the normal adjacent tissue. Thus, therapeutic modulation of the expression or function of this gene is effective in the treatment of breast, ovarian and lung cancers.

Panel 4D Summary: Agl 143/Ag1921/Ag3082 Highest expression of this gene was detected in treated lung fibroblasts (CTs=27-29). Moderate levels of expression were also seen in treated dermal fibroblasts, and lung and dermal microvasculature, and HUVECs. Thus, expression of this gene is useful as a marker of fibroblasts or vasculature. The protein encoded by the transcript plays an important role in the normal homeostasis of these tissues. Therefore, therapeutics designed with this gene or gene product are important for maintaining or restoring normal function to these organs during inflammation associated with asthma, psoriasis, and emphysema.

BH. CG93871-01 and CG93871-05: Weakly Similar to Fibulin-1

Expression of gene CG93871-05 and variant CG93871-01 was assessed using the primer-probe sets Ag1294b, Ag746, and Ag4726, described in Tables BHA, BHB, and BHC. Results of the RTQ-PCR runs are shown in Tables BHD, BHE, BHF, BHG, and BHH. Please note that the probe and primer set Ag4726 is specific to the CG93871-05 variant only.

TABLE BHA
Probe Name Ag1294b

| | | | | SEQ |
|---|---|---|---|---|
| | | | Start | ID |
| Primers | Sequences | Length | Position | No |
| Forward | 5′-cattggcagctacaagtgttc-3′ | 21 | 532 | 1582 |
| Probe | TET-5′-ctgtcgaactggcttccaccttcat-3′- | 25 | 553 | 1583 |
| | TAMRA | | | |
| Reverse | 5′-cctccgacactcgtttacatc-3′ | 21 | 599 | 1584 |

TABLE BHB
Probe Name Ag746

| | | | | SEQ |
|---|---|---|---|---|
| | | | Start | ID |
| Primers | Sequences | Length | Position | No |
| Forward | 5′-gcattggcagctacaagtgt-3′ | 20 | 531 | 1585 |
| Probe | TET-5′-ctgtcgaactggcttccaccttcat-3′- | 25 | 553 | 1586 |
| | TAMRA | | | |
| Reverse | 5′-cctccgacactcgtttacatc-3′ | 21 | 599 | 1587 |

TABLE BHC
Probe Name Ag4726

| | | | | SEQ |
|---|---|---|---|---|
| | | | Start | ID |
| Primers | Sequences | Length | Position | No |
| Forward | 5′-gtgtctgtctggctggaaac-3′ | 20 | 1338 | 1588 |
| Probe | TET-5′-tgcatctctcctgagtgtccttctgg-3′- | 26 | 1364 | 1589 |
| | TAMRA | | | |
| Reverse | 5′-acaagtacagcaatccgtctgt-3′ | 22 | 1408 | 1590 |

TABLE BHD
AI_comprehensive panel_v1.0

| Tissue Name | A |
|---|---|
| 110967 COPD-F | 6.6 |
| 110980 COPD-F | 16.6 |
| 110968 COPD-M | 3.9 |
| 110977 COPD-M | 31.6 |
| 110989 Emphysema-F | 45.1 |
| 110992 Emphysema-F | 7.2 |
| 110993 Emphysema-F | 5.8 |
| 110994 Emphysema-F | 3.3 |
| 110995 Emphysema-F | 2.0 |
| 110996 Emphysema-F | 3.1 |
| 110997 Asthma-M | 3.7 |
| 111001 Asthma-F | 2.8 |
| 111002 Asthma-F | 5.3 |
| 111003 Atopic Asthma-F | 6.1 |
| 111004 Atopic Asthma-F | 3.4 |
| 111005 Atopic Asthma-F | 3.9 |
| 111006 Atopic Asthma-F | 2.4 |
| 111417 Allergy-M | 6.6 |
| 112347 Allergy-M | 3.3 |
| 112349 Normal Lung-F | 3.2 |
| 112357 Normal Lung-F | 100.0 |
| 112354 Normal Lung-M | 58.6 |
| 112374 Crohns-F | 7.5 |
| 112389 Match Control Crohns-F | 3.5 |
| 112375 Crohns-F | 5.1 |
| 112732 Match Control Crohns-F | 0.5 |
| 112725 Crohns-M | 10.6 |
| 112387 Match Control Crohns-M | 3.5 |
| 112378 Crohns-M | 1.7 |
| 112390 Match Control Crohns-M | 55.5 |
| 112726 Crohns-M | 3.6 |
| 112731 Match Control Crohns-M | 13.9 |
| 112380 Ulcer Col-F | 13.7 |
| 112734 Match Control Ulcer Col-F | 5.6 |
| 112384 Ulcer Col-F | 3.9 |
| 112737 Match Control Ulcer Col-F | 3.3 |
| 112386 Ulcer Col-F | 0.0 |
| 112738 Match Control Ulcer Col-F | 0.0 |
| 112381 Ulcer Col-M | 4.2 |
| 112735 Match Control Ulcer Col-M | 18.2 |
| 112382 Ulcer Col-M | 4.2 |
| 112394 Match Control Ulcer Col-M | 0.0 |
| 112383 Ulcer Col-M | 12.2 |
| 112736 Match Control Ulcer Col-M | 2.0 |
| 112423 Psoriasis-F | 3.9 |
| 112427 Match Control Psoriasis-F | 30.8 |
| 112418 Psoriasis-M | 4.6 |
| 112723 Match Control Psoriasis-M | 23.8 |
| 112419 Psoriasis-M | 2.7 |
| 112424 Match Control Psoriasis-M | 1.9 |
| 112420 Psoriasis-M | 4.9 |
| 112425 Match Control Psoriasis-M | 25.9 |
| 104689 (MF) OA Bone-Backus | 12.9 |
| 104690 (MF) Adj “Normal” Bone-Backus | 3.7 |
| 104691 (MF) OA Synovium-Backus | 6.9 |
| 104692 (BA) OA Cartilage-Backus | 21.3 |
| 104694 (BA) OA Bone-Backus | 6.6 |
| 104695 (BA) Adj “Normal” Bone-Backus | 2.3 |
| 104696 (BA) OA Synovium-Backus | 5.7 |
| 104700 (SS) OA Bone-Backus | 6.2 |
| 104701 (SS) Adj “Normal” Bone-Backus | 3.8 |
| 104702 (SS) OA Synovium-Backus | 15.4 |
| 117093 OA Cartilage Rep7 | 18.0 |
| 112672 OA Bone5 | 90.1 |
| 112673 OA Synovium5 | 63.7 |
| 112674 OA Synovial Fluid cells5 | 32.3 |
| 117100 OA Cartilage Rep14 | 3.3 |
| 112756 OA Bone9 | 7.0 |
| 112757 OA Synovium9 | 12.2 |
| 112758 OA Synovial Fluid Cells9 | 3.9 |
| 117125 RA Cartilage Rep2 | 4.6 |
| 113492 Bone2 RA | 2.4 |
| 113493 Synovium2 RA | 1.1 |
| 113494 Syn Fluid Cells RA | 1.4 |
| 113499 Cartilage4 RA | 1.4 |
| 113500 Bone4 RA | 0.5 |
| 113501 Synovium4 RA | 1.7 |
| 113502 Syn Fluid Cells4 RA | 1.8 |
| 113495 Cartilage3 RA | 1.6 |
| 113496 Bone3 RA | 1.1 |
| 113497 Synovium3 RA | 0.0 |
| 113498 Syn Fluid Cells3 RA | 0.6 |
| 117106 Normal Cartilage Rep20 | 4.5 |
| 113663 Bone3 Normal | 6.7 |
| 113664 Synovium3 Normal | 1.2 |
| 113665 Syn Fluid Cells3 Normal | 0.9 |
| 117107 Normal Cartilage Rep22 | 1.3 |
| 113667 Bone4 Normal | 11.8 |
| 113668 Synovium4 Normal | 12.0 |
| 113669 Syn Fluid Cells4 Normal | 10.7 |

Column A - Rel. Exp. (%) Ag1294b, Run 249007981

TABLE BHE
General_screening_panel_v1.4

| Tissue Name | A |
|---|---|
| Adipose | 3.3 |
| Melanoma* Hs688(A).T | 2.7 |
| Melanoma* Hs688(B).T | 2.7 |
| Melanoma* M14 | 4.8 |
| Melanoma* LOXIMVI | 0.1 |
| Melanoma* SK-MEL-5 | 0.4 |
| Squamous cell carcinoma SCC-4 | 0.2 |
| Testis Pool | 4.3 |
| Prostate ca.* (bone met) PC-3 | 1.6 |
| Prostate Pool | 0.5 |
| Placenta | 7.7 |
| Uterus Pool | 0.1 |
| Ovarian ca. OVCAR-3 | 0.7 |
| Ovarian ca. SK-OV-3 | 0.6 |
| Ovarian ca. OVCAR-4 | 0.3 |
| Ovarian ca. OVCAR-5 | 1.1 |
| Ovarian ca. IGROV-1 | 1.4 |
| Ovarian ca. OVCAR-8 | 0.7 |
| Ovary | 5.0 |
| Breast ca. MCF-7 | 0.4 |
| Breast ca. MDA-MB-231 | 0.5 |
| Breast ca. BT 549 | 0.7 |
| Breast ca. T47D | 4.2 |
| Breast ca. MDA-N | 0.2 |
| Breast Pool | 0.8 |
| Trachea | 1.3 |
| Lung | 5.5 |
| Fetal Lung | 1.8 |
| Lung ca. NCI-N417 | 3.6 |
| Lung ca. LX-1 | 0.7 |
| Lung ca. NCI-H146 | 0.8 |
| Lung ca. SHP-77 | 0.3 |
| Lung ca. A549 | 0.8 |
| Lung ca. NCI-H526 | 2.1 |
| Lung ca. NCI-H23 | 0.8 |
| Lung ca. NCI-H460 | 1.2 |
| Lung ca. HOP-62 | 0.5 |
| Lung ca. NCI-H522 | 20.2 |
| Liver | 11.6 |
| Fetal Liver | 61.1 |
| Liver ca. HepG2 | 100.0 |
| Kidney Pool | 6.7 |
| Fetal Kidney | 2.0 |
| Renal ca. 786-0 | 1.7 |
| Renal ca. A498 | 1.3 |
| Renal ca. ACHN | 2.5 |
| Renal ca. UO-31 | 10.6 |
| Renal ca. TK-10 | 41.8 |
| Bladder | 1.8 |
| Gastric ca. (liver met.) NCI-N87 | 2.0 |
| Gastric ca. KATO III | 0.6 |
| Colon ca. SW-948 | 0.6 |
| Colon ca. SW480 | 0.7 |
| Colon ca.* (SW480 met) SW620 | 12.8 |
| Colon ca. HT29 | 0.1 |
| Colon ca. HCT-116 | 3.7 |
| Colon ca. CaCo-2 | 31.9 |
| Colon cancer tissue | 7.9 |
| Colon ca. SW1116 | 1.0 |
| Colon ca. Colo-205 | 0.0 |
| Colon ca. SW-48 | 0.0 |
| Colon Pool | 1.0 |
| Small Intestine Pool | 4.9 |
| Stomach Pool | 3.4 |
| Bone Marrow Pool | 0.0 |
| Fetal Heart | 0.7 |
| Heart Pool | 0.7 |
| Lymph Node Pool | 2.6 |
| Fetal Skeletal Muscle | 1.6 |
| Skeletal Muscle Pool | 1.0 |
| Spleen Pool | 0.4 |
| Thymus Pool | 7.9 |
| CNS cancer (glio/astro) U87-MG | 6.4 |
| CNS cancer (glio/astro) U-118-MG | 1.6 |
| CNS cancer (neuro; met) SK-N-AS | 4.4 |
| CNS cancer (astro) SF-539 | 0.2 |
| CNS cancer (astro) SNB-75 | 4.4 |
| CNS cancer (glio) SNB-19 | 1.1 |
| CNS cancer (glio) SF-295 | 5.1 |
| Brain (Amygdala) Pool | 2.5 |
| Brain (cerebellum) | 7.3 |
| Brain (fetal) | 12.2 |
| Brain (Hippocampus) Pool | 1.9 |
| Cerebral Cortex Pool | 2.6 |
| Brain (Substantia nigra) Pool | 2.1 |
| Brain (Thalamus) Pool | 3.9 |
| Brain (whole) | 8.5 |
| Spinal Cord Pool | 1.9 |
| Adrenal Gland | 2.6 |
| Pituitary gland Pool | 0.6 |
| Salivary Gland | 0.9 |
| Thyroid (female) | 1.8 |
| Pancreatic ca. CAPAN2 | 0.0 |
| Pancreas Pool | 1.1 |

Column A - Rel. Exp. (%) Ag4726, Run 222842378

TABLE BHF
Panel 1.2

| Tissue Name | A | B |
|---|---|---|
| Endothelial cells | 12.3 | 5.9 |
| Heart (Fetal) | 0.0 | 0.0 |
| Pancreas | 0.0 | 0.0 |
| Pancreatic ca. CAPAN 2 | 0.0 | 0.0 |
| Adrenal gland | 0.0 | 0.2 |
| Thyroid | 0.1 | 0.0 |
| Salivary gland | 0.0 | 0.0 |
| Pituitary gland | 0.2 | 0.1 |
| Brain (fetal) | 2.4 | 16.0 |
| Brain (whole) | 0.0 | 0.3 |
| Brain (amygdala) | 0.0 | 0.0 |
| Brain (cerebellum) | 0.0 | 0.0 |
| Brain (hippocampus) | 0.0 | 0.0 |
| Brain (thalamus) | 0.0 | 0.0 |
| Cerebral Cortex | 0.0 | 0.0 |
| Spinal cord | 0.0 | 0.0 |
| glio/astro U87-MG | 0.0 | 0.0 |
| glio/astro U-118-MG | 0.0 | 0.0 |
| astrocytoma SW1783 | 0.0 | 0.0 |
| neuro*; met SK-N-AS | 0.0 | 0.2 |
| astrocytoma SF-539 | 0.0 | 0.0 |
| astrocytoma SNB-75 | 0.0 | 0.0 |
| glioma SNB-19 | 0.0 | 0.0 |
| glioma U251 | 0.0 | 0.0 |
| glioma SF-295 | 0.0 | 0.0 |
| Heart | 0.0 | 0.0 |
| Skeletal muscle | 0.0 | 0.0 |
| Bone marrow | 0.0 | 0.0 |
| Thymus | 1.2 | 2.8 |
| Spleen | 0.0 | 0.0 |
| Lymph node | 0.0 | 0.0 |
| Colorectal | 0.0 | 0.0 |
| Stomach | 0.0 | 0.0 |
| Small intestine | 0.0 | 0.0 |
| Colon ca. SW480 | 0.0 | 0.0 |
| Colon ca.* SW620 (SW480 met) | 1.1 | 1.9 |
| Colon ca. HT29 | 0.0 | 0.0 |
| Colon ca. HCT-116 | 0.0 | 0.0 |
| Colon ca. CaCo-2 | 46.3 | 56.6 |
| CC Well to Mod Diff (ODO3866) | 0.0 | 0.0 |
| Colon ca. HCC-2998 | 0.0 | 0.0 |
| Gastric ca. (liver met) NCI-N87 | 0.0 | 0.0 |
| Bladder | 0.0 | 0.0 |
| Trachea | 0.0 | 0.0 |
| Kidney | 0.0 | 0.0 |
| Kidney (fetal) | 0.1 | 0.9 |
| Renal ca. 786-0 | 0.0 | 0.0 |
| Renal ca. A498 | 0.0 | 0.0 |
| Renal ca. RXF 393 | 0.0 | 0.0 |
| Renal ca. ACHN | 0.0 | 0.0 |
| Renal ca. UO-31 | 0.0 | 0.0 |
| Renal ca. TK-10 | 0.0 | 0.0 |
| Liver | 32.8 | 53.2 |
| Liver (fetal) | 72.7 | 100.0 |
| Liver ca. (hepatoblast) HepG2 | 100.0 | 94.0 |
| Lung | 0.0 | 0.0 |
| Lung (fetal) | 0.0 | 0.0 |
| Lung ca. (small cell) LX-1 | 0.0 | 0.0 |
| Lung ca. (small cell) NCI-H69 | 0.0 | 0.0 |
| Lung ca. (s. cell var.) SHP-77 | 0.0 | 0.0 |
| Lung ca. (large cell) NCI-H460 | 0.0 | 0.0 |
| Lung ca. (non-sm. cell) A549 | 0.0 | 0.0 |
| Lung ca. (non-s. cell) NCI-H23 | 0.0 | 0.0 |
| Lung ca. (non-s. cell) HOP-62 | 0.0 | 0.0 |
| Lung ca. (non-s. cl) NCI-H522 | 63.7 | 90.1 |
| Lung ca. (squam.) SW 900 | 0.0 | 0.0 |
| Lung ca. (squam.) NCI-H596 | 0.0 | 0.0 |
| Mammary gland | 0.7 | 3.6 |
| Breast ca.* (pl. ef) MCF-7 | 0.0 | 0.0 |
| Breast ca.* (pl. ef) MDA-MB-231 | 0.0 | 0.0 |
| Breast ca.* (pl. ef) T47D | 0.0 | 0.0 |
| Breast ca. BT-549 | 0.0 | 0.0 |
| Breast ca. MDA-N | 0.0 | 0.0 |
| Ovary | 0.5 | 11.7 |
| Ovarian ca. OVCAR-3 | 0.0 | 0.0 |
| Ovarian ca. OVCAR-4 | 0.0 | 0.0 |
| Ovarian ca. OVCAR-5 | 0.0 | 0.0 |
| Ovarian ca. OVCAR-8 | 0.0 | 0.0 |
| Ovarian ca. IGROV-1 | 0.0 | 0.0 |
| Ovarian ca. (ascites) SK-OV-3 | 0.0 | 0.0 |
| Uterus | 0.0 | 0.0 |
| Placenta | 34.4 | 39.5 |
| Prostate | 0.0 | 0.0 |
| Prostate ca.* (bone met) PC-3 | 0.0 | 0.0 |
| Testis | 1.0 | 3.5 |
| Melanoma Hs688(A).T | 0.0 | 0.0 |
| Melanoma* (met) Hs688(B).T | 0.0 | 0.0 |
| Melanoma UACC-62 | 0.0 | 0.0 |
| Melanoma M14 | 0.0 | 0.0 |
| Melanoma LOX IMVI | 0.0 | 0.0 |
| Melanoma* (met) SK-MEL-5 | 0.0 | 0.0 |

Column A - Rel. Exp. (%) Ag746, Run 115163442
Column B - Rel. Exp. (%) Ag746, Run 119442272

TABLE BHG
Panel 2D

| Tissue Name | A | B |
|---|---|---|
| Normal Colon | 18.3 | 21.8 |
| CC Well to Mod Diff (ODO3866) | 16.5 | 23.7 |
| CC Margin (ODO3866) | 3.1 | 0.0 |
| CC Gr.2 rectosigmoid (ODO3868) | 0.0 | 0.8 |
| CC Margin (ODO3868) | 0.5 | 2.0 |
| CC Mod Diff (ODO3920) | 1.2 | 2.3 |
| CC Margin (ODO3920) | 1.3 | 2.6 |
| CC Gr.2 ascend colon (ODO3921) | 3.4 | 4.4 |
| CC Margin (ODO3921) | 1.3 | 0.0 |
| CC from Partial Hepatectomy | 8.4 | 1.9 |
| (ODO4309) Mets | | |
| Liver Margin (ODO4309) | 49.7 | 41.5 |
| Colon mets to lung (OD04451-01) | 0.3 | 5.3 |
| Lung Margin (OD04451-02) | 0.0 | 1.8 |
| Normal Prostate 6546-1 | 9.1 | 12.1 |
| Prostate Cancer (OD04410) | 2.0 | 9.7 |
| Prostate Margin (OD04410) | 16.8 | 20.3 |
| Prostate Cancer (OD04720-01) | 13.5 | 14.4 |
| Prostate Margin (OD04720-02) | 14.0 | 22.4 |
| Normal Lung | 6.8 | 11.7 |
| Lung Met to Muscle (ODO4286) | 1.8 | 0.7 |
| Muscle Margin (ODO4286) | 11.5 | 13.1 |
| Lung Malignant Cancer (OD03126) | 1.5 | 6.0 |
| Lung Margin (OD03126) | 4.8 | 2.4 |
| Lung Cancer (OD04404) | 4.2 | 2.3 |
| Lung Margin (OD04404) | 9.0 | 10.4 |
| Lung Cancer (OD04565) | 0.3 | 0.0 |
| Lung Margin (OD04565) | 0.4 | 0.3 |
| Lung Cancer (OD04237-01) | 10.7 | 11.1 |
| Lung Margin (OD04237-02) | 4.9 | 5.4 |
| Ocular Mel Met to Liver (ODO4310) | 10.5 | 11.9 |
| Liver Margin (ODO4310) | 22.4 | 32.8 |
| Melanoma Metastasis | 0.0 | 0.0 |
| Lung Margin (OD04321) | 0.6 | 0.0 |
| Normal Kidney | 5.3 | 5.3 |
| Kidney Ca, Nuclear grade 2 (OD04338) | 39.8 | 43.8 |
| Kidney Margin (OD04338) | 4.8 | 6.4 |
| Kidney Ca Nuclear grade 1/2 | 3.0 | 0.3 |
| (OD04339) | | |
| Kidney Margin (OD04339) | 5.4 | 10.0 |
| Kidney Ca, Clear cell type (OD04340) | 18.2 | 19.2 |
| Kidney Margin (OD04340) | 9.0 | 10.4 |
| Kidney Ca, Nuclear grade 3 (OD04348) | 5.2 | 8.3 |
| Kidney Margin (OD04348) | 6.9 | 4.7 |
| Kidney Cancer (OD04622-01) | 41.8 | 45.4 |
| Kidney Margin (OD04622-03) | 1.9 | 1.4 |
| Kidney Cancer (OD04450-01) | 9.2 | 6.2 |
| Kidney Margin (OD04450-03) | 10.2 | 9.0 |
| Kidney Cancer 8120607 | 2.2 | 1.7 |
| Kidney Margin 8120608 | 6.5 | 6.4 |
| Kidney Cancer 8120613 | 2.2 | 0.7 |
| Kidney Margin 8120614 | 6.3 | 3.0 |
| Kidney Cancer 9010320 | 10.9 | 16.5 |
| Kidney Margin 9010321 | 9.0 | 11.3 |
| Normal Uterus | 4.3 | 6.3 |
| Uterine Cancer 064011 | 13.4 | 17.7 |
| Normal Thyroid | 9.1 | 14.9 |
| Thyroid Cancer | 6.4 | 5.9 |
| Thyroid Cancer A302152 | 4.4 | 5.1 |
| Thyroid Margin A302153 | 12.0 | 22.1 |
| Normal Breast | 9.9 | 14.3 |
| Breast Cancer | 0.4 | 0.2 |
| Breast Cancer (OD04590-01) | 5.3 | 3.9 |
| Breast Cancer Mets (OD04590-03) | 4.0 | 10.4 |
| Breast Cancer Metastasis | 7.2 | 4.4 |
| Breast Cancer | 5.2 | 3.3 |
| Breast Cancer | 12.1 | 18.6 |
| Breast Cancer 9100266 | 2.7 | 5.3 |
| Breast Margin 9100265 | 5.0 | 5.8 |
| Breast Cancer A209073 | 0.5 | 1.8 |
| Breast Margin A209073 | 1.7 | 0.4 |
| Normal Liver | 39.5 | 47.0 |
| Liver Cancer | 4.2 | 0.6 |
| Liver Cancer 1025 | 66.4 | 74.2 |
| Liver Cancer 1026 | 36.1 | 42.6 |
| Liver Cancer 6004-T | 100.0 | 100.0 |
| Liver Tissue 6004-N | 22.8 | 34.4 |
| Liver Cancer 6005-T | 39.2 | 35.4 |
| Liver Tissue 6005-N | 33.2 | 38.2 |
| Normal Bladder | 6.6 | 4.9 |
| Bladder Cancer | 1.0 | 4.8 |
| Bladder Cancer | 2.6 | 0.7 |
| Bladder Cancer (OD04718-01) | 0.0 | 0.7 |
| Bladder Normal Adjacent | 3.5 | 14.4 |
| (OD04718-03) | | |
| Normal Ovary | 50.7 | 47.3 |
| Ovarian Cancer | 10.2 | 7.4 |
| Ovarian Cancer (OD04768-07) | 73.7 | 80.7 |
| Ovary Margin (OD04768-08) | 2.6 | 0.8 |
| Normal Stomach | 2.9 | 2.9 |
| Gastric Cancer 9060358 | 0.0 | 1.1 |
| Stomach Margin 9060359 | 2.4 | 0.3 |
| Gastric Cancer 9060395 | 0.5 | 1.1 |
| Stomach Margin 9060394 | 5.2 | 2.0 |
| Gastric Cancer 9060397 | 3.4 | 7.0 |
| Stomach Margin 9060396 | 1.4 | 0.0 |
| Gastric Cancer 064005 | 1.3 | 6.0 |

Column A - Rel. Exp. (%) Ag746, Run 147127131
Column B - Rel. Exp. (%) Ag746, Run 148019631

TABLE BHH
Panel 4D

| Tissue Name | A | B |
|---|---|---|
| Secondary Th1 act | 10.9 | 7.7 |
| Secondary Th2 act | 6.4 | 8.0 |
| Secondary Tr1 act | 11.3 | 9.3 |
| Secondary Th1 rest | 3.4 | 2.7 |
| Secondary Th2 rest | 1.5 | 2.5 |
| Secondary Tr1 rest | 1.4 | 2.0 |
| Primary Th1 act | 48.0 | 46.0 |
| Primary Th2 act | 38.7 | 27.7 |
| Primary Tr1 act | 72.2 | 55.5 |
| Primary Th1 rest | 3.1 | 2.3 |
| Primary Th2 rest | 1.0 | 0.8 |
| Primary Tr1 rest | 1.1 | 0.5 |
| CD45RA CD4 lymphocyte act | 2.9 | 1.8 |
| CD45RO CD4 lymphocyte act | 18.6 | 12.2 |
| CD8 lymphocyte act | 17.8 | 6.8 |
| Secondary CD8 lymphocyte rest | 6.8 | 6.0 |
| Secondary CD8 lymphocyte act | 5.5 | 4.1 |
| CD4 lymphocyte none | 0.0 | 0.2 |
| 2ry Th1/Th2/Tr1 anti-CD95 | 2.9 | 3.1 |
| CH11 | | |
| LAK cells rest | 1.4 | 0.3 |
| LAK cells IL-2 | 3.8 | 2.2 |
| LAK cells IL-2 + IL-12 | 3.0 | 0.8 |
| LAK cells IL-2 + IFN gamma | 2.0 | 1.7 |
| LAK cells IL-2 + IL-18 | 0.5 | 0.2 |
| LAK cells PMA/ionomycin | 0.7 | 1.3 |
| NK Cells IL-2 rest | 0.7 | 0.7 |
| Two Way MLR 3 day | 1.1 | 2.5 |
| Two Way MLR 5 day | 2.5 | 2.8 |
| Two Way MLR 7 day | 4.5 | 5.0 |
| PBMC rest | 0.0 | 0.0 |
| PBMC PWM | 41.8 | 29.1 |
| PBMC PHA-L | 34.4 | 21.8 |
| Ramos (B cell) none | 4.7 | 2.4 |
| Ramos (B cell) ionomycin | 9.2 | 5.8 |
| B lymphocytes PWM | 51.8 | 51.4 |
| B lymphocytes CD40L and IL-4 | 10.2 | 12.3 |
| EOL-1 dbcAMP | 0.3 | 0.2 |
| EOL-1 dbcAMP | 0.4 | 1.8 |
| PMA/ionomycin | | |
| Dendritic cells none | 6.7 | 3.8 |
| Dendritic cells LPS | 4.7 | 3.1 |
| Dendritic cells anti-CD40 | 6.0 | 5.6 |
| Monocytes rest | 0.0 | 0.0 |
| Monocytes LPS | 0.7 | 0.8 |
| Macrophages rest | 19.8 | 9.9 |
| Macrophages LPS | 0.7 | 0.5 |
| HUVEC none | 9.3 | 10.2 |
| HUVEC starved | 19.2 | 13.1 |
| HUVEC IL-1beta | 4.1 | 1.7 |
| HUVEC IFN gamma | 21.0 | 13.7 |
| HUVEC TNF alpha + IFN gamma | 2.8 | 0.6 |
| HUVEC TNF alpha + IL4 | 30.8 | 25.7 |
| HUVEC IL-11 | 11.6 | 7.3 |
| Lung Microvascular EC none | 24.1 | 20.0 |
| Lung Microvascular EC TNFalpha + IL- | 8.0 | 12.2 |
| 1beta | | |
| Microvascular Dermal EC none | 64.6 | 45.7 |
| Microsvasular Dermal EC TNFalpha + IL- | 18.4 | 11.7 |
| 1beta | | |
| Bronchial epithelium | 5.2 | 5.4 |
| TNFalpha + IL1beta | | |
| Small airway epithelium none | 4.0 | 3.2 |
| Small airway epithelium TNFalpha + IL- | 8.2 | 4.5 |
| 1beta | | |
| Coronery artery SMC rest | 5.8 | 6.3 |
| Coronery artery SMC | 4.5 | 5.1 |
| TNFalpha + IL-1beta | | |
| Astrocytes rest | 0.8 | 0.5 |
| Astrocytes TNFalpha + IL-1beta | 3.6 | 1.9 |
| KU-812 (Basophil) rest | 16.0 | 11.1 |
| KU-812 (Basophil) PMA/ionomycin | 12.3 | 9.5 |
| CCD1106 (Keratinocytes) none | 0.0 | 0.5 |
| 93580 CCD1106 (Keratinocytes) TNFa and | 0.7 | 0.4 |
| IFNg | | |
| Liver cirrhosis | 8.4 | 3.8 |
| Lupus kidney | 2.0 | 3.2 |
| NCI-H292 none | 21.9 | 25.7 |
| NCI-H292 IL-4 | 15.7 | 12.3 |
| NCI-H292 IL-9 | 20.6 | 14.7 |
| NCI-H292 IL-13 | 8.3 | 5.7 |
| NCI-H292 IFN gamma | 5.1 | 8.2 |
| HPAEC none | 18.7 | 23.8 |
| HPAEC TNF alpha + IL-1 beta | 11.9 | 12.9 |
| Lung fibroblast none | 15.7 | 13.5 |
| Lung fibroblast TNF alpha + IL-1 beta | 6.9 | 4.7 |
| Lung fibroblast IL-4 | 25.0 | 16.6 |
| Lung fibroblast IL-9 | 14.7 | 15.8 |
| Lung fibroblast IL-13 | 40.3 | 32.5 |
| Lung fibroblast IFN gamma | 15.4 | 17.4 |
| Dermal fibroblast CCD1070 rest | 0.5 | 0.9 |
| Dermal fibroblast CCD1070 TNF alpha | 0.9 | 0.8 |
| Dermal fibroblast CCD1070 IL-1 beta | 0.6 | 0.6 |
| Dermal fibroblast IFN gamma | 32.1 | 18.4 |
| Dermal fibroblast IL-4 | 100.0 | 100.0 |
| IBD Colitis 2 | 0.0 | 0.0 |
| IBD Crohn's | 0.3 | 0.8 |
| Colon | 1.4 | 0.5 |
| Lung | 0.5 | 0.8 |
| Thymus | 2.9 | 4.3 |
| Kidney | 65.5 | 47.3 |

Column A - Rel. Exp. (%) Ag1294b, Run 138944262
Column B - Rel. Exp. (%) Ag1294b, Run 139408252

AI_comprehensive panel_v1.0 Summary: Ag1294b Expression of this gene in this panel confirmed expression of this gene in cells involved in the immune response. Highest expression of this gene was seen in normal lung (CT=30.5). Please see Panel 4D for discussion of utility of this gene in inflammation.

General_screeningpanel_v1.4 Summary: Ag4726 Highest expression of this gene was seen in a liver cancer cell line (CTs=30), with moderate levels of expression seen in fetal and adult liver, and cell lines derived from colon, renal and lung cancers. Thus, expression of this gene is useful as a marker to differentiate liver derived tissue from other samples on this panel.

Panel 1.2 Summary: Ag746 Highest expression of this gene was detected in a liver cancer cell line (CTs=27). High levels of expression were also seen in fetal and adult liver tissue, a colon cancer cell line and a lung cancer cell line. Thus, expression of this gene is useful as a marker to differentiate liver derived samples, the colon cancer cell line and the lung cancer cell line from other samples on this panel. Expression of this gene is also useful as a diagnostic marker to detect the presence of colon and lung cancers.

Moderate expression was also seen in the fetal brain, placenta, and endothelial cells.

Panel 2D Summary: Ag746 Highest expression of this gene was detected in a liver cancer sample (CTs=31). The prominent expression in liver derived tissue was consistent with the results in Panel 1.2. Moderate levels of expression were also evident in samples from ovarian cancer and kidney cancer. Expression of this gene was higher in these cancers than in the normal adjacent tissue. Thus, expression of this gene is useful as a marker to differentiate between liver derived samples and other samples on this panel and as a marker to detect the presence of liver, kidney, and ovarian cancer. Therapeutic modulation of the expression or function of this gene or its protein product is useful in the treatment of liver, kidney, and ovarian cancers.

Panel 4D Summary: Ag1294b Highest expression of this gene was detected in IL-4 treated dermal fibroblasts (CTs=30). In addition, this gene was expressed at moderate levels in IFN gamma stimulated dermal fibroblasts, activated lung fibroblasts, HPAECs, lung and dermal microvasculature, activated small airway and bronchial epithelium, activated NCI-H292 cells, acutely activated T cells, and activated B cells.

Based on these levels of expression in T cells, activated B cells and cells in lung and skin, therapeutics that block the function of this gene product are useful as therapeutics that reduce or eliminate the symptoms in patients with autoimmune and inflammatory diseases in which activated B cells present antigens in the generation of the aberrant immune response and in treating T-cell mediated diseases, including Crohn's disease, ulcerative colitis, multiple sclerosis, chronic obstructive pulmonary disease, asthma, allergy, emphysema, rheumatoid arthritis, or psoriasis.

BE. CG94946-01: Agrin Precursor

Expression of gene CG94946-01 was assessed using the primer-probe sets Ag3605 and Ag3974, described in Tables BIA and BIB. Results of the RTQ-PCR runs are shown in Tables BIC, BID, BIE and BIF.

TABLE BIA
Probe Name Ag36O5

| | | | | |
|---|---|---|---|---|
| | +HL,6 | | | SEQ |
| | | | Start | ID |
| Primers | Sequences | Length | Position | No |

| | | | | |
|---|---|---|---|---|
| Forward | 5′-gaccccaagtcagaactgttc-3′ | 21 | 3514 | 1591 |
| Probe | TET-5′-attgagagcaccctggacgacctctt-3′- | 26 | 3553 | 1592 |
| | TAMRA | | | |
| Reverse | 5′-gaaatccttcttgacgtctgaa-3′ | 22 | 3585 | 1593 |

TABLE BIB
Probe Name Ag3974

| | +HL,6 | | | SEQ |
|---|---|---|---|---|
| | | | Start | ID |
| Primers | Sequences | Length | Position | No |
| Forward | 5′-gacaccaggatcttctttgtga-3′ | 22 | 379 | 1594 |
| Probe | TET-5′-catacctgtggccagcccacaag-3′-TAMRA | 23 | 413 | 1595 |
| Reverse | 5′-gagttgagcatcagctcgtt-3′ | 20 | 436 | 1596 |

TABLE BIC
General_screening_panel_v1.4

| Tissue Name | A | B |
|---|---|---|
| Adipose | 1.4 | 1.5 |
| Melanoma* Hs688(A).T | 2.6 | 3.2 |
| Melanoma* Hs688(B).T | 4.6 | 4.2 |
| Melanoma* M14 | 6.7 | 6.4 |
| Melanoma* LOXIMVI | 4.8 | 4.0 |
| Melanoma* SK-MEL-5 | 2.6 | 4.2 |
| Squamous cell carcinoma SCC-4 | 9.0 | 8.4 |
| Testis Pool | 1.3 | 1.1 |
| Prostate ca.* (bone met) PC-3 | 21.0 | 24.8 |
| Prostate Pool | 0.9 | 0.8 |
| Placenta | 0.9 | 1.3 |
| Uterus Pool | 0.4 | 0.4 |
| Ovarian ca. OVCAR-3 | 77.4 | 66.9 |
| Ovarian ca. SK-OV-3 | 42.0 | 36.3 |
| Ovarian ca. OVCAR-4 | 9.5 | 12.7 |
| Ovarian ca. OVCAR-5 | 39.2 | 44.4 |
| Ovarian ca. IGROV-1 | 22.1 | 27.7 |
| Ovarian ca. OVCAR-8 | 18.0 | 14.9 |
| Ovary | 1.5 | 1.9 |
| Breast ca. MCF-7 | 7.7 | 9.7 |
| Breast ca. MDA-MB-231 | 22.7 | 31.2 |
| Breast ca. BT 549 | 13.2 | 10.1 |
| Breast ca. T47D | 100.0 | 100.0 |
| Breast ca. MDA-N | 4.8 | 4.2 |
| Breast Pool | 1.6 | 1.6 |
| Trachea | 2.8 | 2.6 |
| Lung | 0.2 | 0.1 |
| Fetal Lung | 11.4 | 8.3 |
| Lung ca. NCI-N417 | 1.4 | 0.7 |
| Lung ca. LX-1 | 10.5 | 11.0 |
| Lung ca. NCI-H146 | 0.1 | 0.1 |
| Lung ca. SHP-77 | 1.1 | 0.8 |
| Lung ca. A549 | 15.6 | 10.4 |
| Lung ca. NCI-H526 | 5.4 | 1.6 |
| Lung ca. NCI-H23 | 18.9 | 20.6 |
| Lung ca. NCI-H460 | 11.5 | 9.3 |
| Lung ca. HOP-62 | 23.7 | 23.0 |
| Lung ca. NCI-H522 | 1.8 | 2.3 |
| Liver | 0.5 | 0.6 |
| Fetal Liver | 0.8 | 1.4 |
| Liver ca. HepG2 | 15.5 | 12.6 |
| Kidney Pool | 1.5 | 2.5 |
| Fetal Kidney | 5.8 | 4.6 |
| Renal ca. 786-0 | 46.3 | 39.5 |
| Renal ca. A498 | 13.8 | 7.9 |
| Renal ca. ACHN | 14.3 | 15.9 |
| Renal ca. UO-31 | 41.5 | 38.7 |
| Renal ca. TK-10 | 19.3 | 16.4 |
| Bladder | 8.4 | 9.0 |
| Gastric ca. (liver met.) NCI-N87 | 87.7 | 80.7 |
| Gastric ca. KATO III | 17.8 | 17.7 |
| Colon ca. SW-948 | 9.2 | 7.8 |
| Colon ca. SW480 | 25.0 | 32.3 |
| Colon ca.* (SW480 met) SW620 | 5.0 | 4.6 |
| Colon ca. HT29 | 26.8 | 30.6 |
| Colon ca. HCT-116 | 4.9 | 5.8 |
| Colon ca. CaCo-2 | 13.6 | 10.4 |
| Colon cancer tissue | 10.2 | 10.0 |
| Colon ca. SW1116 | 5.1 | 3.6 |
| Colon ca. Colo-205 | 1.8 | 1.5 |
| Colon ca. SW-48 | 1.2 | 0.7 |
| Colon Pool | 1.9 | 1.3 |
| Small Intestine Pool | 0.6 | 1.0 |
| Stomach Pool | 1.5 | 1.2 |
| Bone Marrow Pool | 0.6 | 0.5 |
| Fetal Heart | 1.4 | 1.0 |
| Heart Pool | 0.7 | 0.8 |
| Lymph Node Pool | 2.1 | 2.0 |
| Fetal Skeletal Muscle | 0.9 | 0.5 |
| Skeletal Muscle Pool | 0.4 | 0.5 |
| Spleen Pool | 0.7 | 0.7 |
| Thymus Pool | 1.8 | 2.2 |
| CNS cancer (glio/astro) U87-MG | 5.9 | 6.0 |
| CNS cancer (glio/astro) U-118-MG | 11.7 | 11.2 |
| CNS cancer (neuro; met) SK-N-AS | 1.2 | 0.9 |
| CNS cancer (astro) SF-539 | 6.7 | 5.0 |
| CNS cancer (astro) SNB-75 | 22.8 | 32.3 |
| CNS cancer (glio) SNB-19 | 25.0 | 20.2 |
| CNS cancer (glio) SF-295 | 35.1 | 38.2 |
| Brain (Amygdala) Pool | 1.7 | 1.3 |
| Brain (cerebellum) | 1.4 | 1.0 |
| Brain (fetal) | 7.0 | 2.8 |
| Brain (Hippocampus) Pool | 1.6 | 0.9 |
| Cerebral Cortex Pool | 1.9 | 0.9 |
| Brain (Substantia nigra) Pool | 2.8 | 1.7 |
| Brain (Thalamus) Pool | 2.7 | 1.6 |
| Brain (whole) | 3.4 | 1.1 |
| Spinal Cord Pool | 1.8 | 1.4 |
| Adrenal Gland | 0.2 | 0.4 |
| Pituitary gland Pool | 0.3 | 0.2 |
| Salivary Gland | 1.1 | 1.3 |
| Thyroid (female) | 3.3 | 3.7 |
| Pancreatic ca. CAPAN2 | 23.7 | 27.7 |
| Pancreas Pool | 3.0 | 4.1 |

Column A - Rel. Exp. (%) Ag3605, Run 213406184
Column B - Rel. Exp. (%) Ag3974, Run 217508632

TABLE BID
Panel 2.2

| Tissue Name | A |
|---|---|
| Normal Colon | 4.7 |
| Colon cancer (OD06064) | 6.3 |
| Colon Margin (OD06064) | 2.3 |
| Colon cancer (OD06159) | 2.1 |
| Colon Margin (OD06159) | 1.8 |
| Colon cancer (OD06297-04) | 2.0 |
| Colon Margin (OD06297-05) | 3.0 |
| CC Gr.2 ascend colon (ODO3921) | 3.6 |
| CC Margin (ODO3921) | 1.3 |
| Colon cancer metastasis (OD06104) | 1.1 |
| Lung Margin (OD06104) | 1.0 |
| Colon mets to lung (OD04451-01) | 4.5 |
| Lung Margin (OD04451-02) | 6.7 |
| Normal Prostate | 2.1 |
| Prostate Cancer (OD04410) | 3.9 |
| Prostate Margin (OD04410) | 3.4 |
| Normal Ovary | 7.6 |
| Ovarian cancer (OD06283-03) | 27.9 |
| Ovarian Margin (OD06283-07) | 2.0 |
| Ovarian Cancer | 16.0 |
| Ovarian cancer (OD06145) | 10.1 |
| Ovarian Margin (OD06145) | 8.2 |
| Ovarian cancer (OD06455-03) | 28.9 |
| Ovarian Margin (OD06455-07) | 1.9 |
| Normal Lung | 2.9 |
| Invasive poor diff. lung adeno (ODO4945-01 | 9.4 |
| Lung Margin (ODO4945-03) | 7.9 |
| Lung Malignant Cancer (OD03126) | 7.5 |
| Lung Margin (OD03126) | 7.0 |
| Lung Cancer (OD05014A) | 17.0 |
| Lung Margin (OD05014B) | 11.7 |
| Lung cancer (OD06081) | 12.2 |
| Lung Margin (OD06081) | 2.4 |
| Lung Cancer (OD04237-01) | 1.8 |
| Lung Margin (OD04237-02) | 16.2 |
| Ocular Mel Met to Liver (ODO4310) | 8.4 |
| Liver Margin (ODO4310) | 2.9 |
| Melanoma Metastasis | 4.0 |
| Lung Margin (OD04321) | 3.8 |
| Normal Kidney | 10.9 |
| Kidney Ca, Nuclear grade 2 (OD04338) | 35.4 |
| Kidney Margin (OD04338) | 20.4 |
| Kidney Ca Nuclear grade 1/2 (OD04339) | 52.9 |
| Kidney Margin (OD04339) | 16.6 |
| Kidney Ca, Clear cell type (OD04340) | 16.6 |
| Kidney Margin (OD04340) | 7.4 |
| Kidney Ca, Nuclear grade 3 (OD04348) | 11.2 |
| Kidney Margin (OD04348) | 100.0 |
| Kidney malignant cancer (OD06204B) | 21.6 |
| Kidney normal adjacent tissue (OD06204E) | 23.2 |
| Kidney Cancer (OD04450-01) | 53.2 |
| Kidney Margin (OD04450-03) | 21.3 |
| Kidney Cancer 8120613 | 1.1 |
| Kidney Margin 8120614 | 14.1 |
| Kidney Cancer 9010320 | 20.3 |
| Kidney Margin 9010321 | 15.0 |
| Kidney Cancer 8120607 | 71.7 |
| Kidney Margin 8120608 | 12.2 |
| Normal Uterus | 7.3 |
| Uterine Cancer 064011 | 6.9 |
| Normal Thyroid | 4.3 |
| Thyroid Cancer | 27.0 |
| Thyroid Cancer A302152 | 19.1 |
| Thyroid Margin A302153 | 8.1 |
| Normal Breast | 14.0 |
| Breast Cancer | 13.4 |
| Breast Cancer | 35.1 |
| Breast Cancer (OD04590-01) | 31.6 |
| Breast Cancer Mets (OD04590-03) | 8.7 |
| Breast Cancer Metastasis | 13.3 |
| Breast Cancer | 21.5 |
| Breast Cancer 9100266 | 17.7 |
| Breast Margin 9100265 | 16.5 |
| Breast Cancer A209073 | 13.2 |
| Breast Margin A209073 | 35.4 |
| Breast cancer (OD06083) | 24.5 |
| Breast cancer node metastasis (OD06083) | 21.5 |
| Normal Liver | 5.0 |
| Liver Cancer 1026 | 15.5 |
| Liver Cancer 1025 | 12.2 |
| Liver Cancer 6004-T | 7.8 |
| Liver Tissue 6004-N | 6.1 |
| Liver Cancer 6005-T | 25.0 |
| Liver Tissue 6005-N | 12.4 |
| Liver Cancer | 12.9 |
| Normal Bladder | 14.2 |
| Bladder Cancer | 9.5 |
| Bladder Cancer | 12.2 |
| Normal Stomach | 8.7 |
| Gastric Cancer 9060397 | 8.9 |
| Stomach Margin 9060396 | 7.4 |
| Gastric Cancer 9060395 | 7.0 |
| Stomach Margin 9060394 | 7.5 |
| Gastric Cancer 064005 | 6.9 |

Column A - Rel. Exp. (%) Ag3605, Run 173764229

TABLE BIE
Panel 4.1D

| Tissue Name | A | B |
|---|---|---|
| Secondary Th1 act | 1.0 | 1.2 |
| Secondary Th2 act | 5.1 | 8.0 |
| Secondary Tr1 act | 2.5 | 3.5 |
| Secondary Th1 rest | 0.0 | 0.7 |
| Secondary Th2 rest | 0.4 | 0.2 |
| Secondary Tr1 rest | 0.4 | 1.2 |
| Primary Th1 act | 3.6 | 3.2 |
| Primary Th2 act | 1.1 | 2.0 |
| Primary Tr1 act | 3.4 | 2.9 |
| Primary Th1 rest | 0.9 | 0.4 |
| Primary Th2 rest | 0.5 | 0.2 |
| Primary Tr1 rest | 0.2 | 0.3 |
| CD45RA CD4 lymphocyte act | 43.5 | 22.7 |
| CD45RO CD4 lymphocyte act | 5.0 | 5.5 |
| CD8 lymphocyte act | 3.9 | 3.3 |
| Secondary CD8 lymphocyte rest | 3.7 | 3.3 |
| Secondary CD8 lymphocyte act | 3.3 | 3.5 |
| CD4 lymphocyte none | 0.3 | 0.1 |
| 2ry Th1/Th2/Tr1 anti-CD95 CH11 | 0.3 | 0.4 |
| LAK cells rest | 5.0 | 6.4 |
| LAK cells IL-2 | 2.8 | 1.7 |
| LAK cells IL-2 + IL-12 | 1.4 | 1.8 |
| LAK cells IL-2 + IFN gamma | 2.3 | 1.1 |
| LAK cells IL-2 + IL-18 | 2.9 | 1.6 |
| LAK cells PMA/ionomycin | 6.8 | 4.6 |
| NK Cells IL-2 rest | 1.6 | 1.9 |
| Two Way MLR 3 day | 10.7 | 12.4 |
| Two Way MLR 5 day | 6.5 | 5.3 |
| Two Way MLR 7 day | 4.3 | 4.0 |
| PBMC rest | 0.0 | 0.6 |
| PBMC PWM | 5.0 | 4.9 |
| PBMC PHA-L | 5.5 | 3.4 |
| Ramos (B cell) none | 0.4 | 0.4 |
| Ramos (B cell) ionomycin | 0.2 | 0.2 |
| B lymphocytes PWM | 2.4 | 3.0 |
| B lymphocytes CD40L and IL-4 | 1.5 | 3.7 |
| EOL-1 dbcAMP | 3.4 | 3.1 |
| EOL-1 dbcAMP PMA/ionomycin | 18.3 | 8.0 |
| Dendritic cells none | 14.8 | 9.0 |
| Dendritic cells LPS | 48.3 | 32.8 |
| Dendritic cells anti-CD40 | 9.7 | 8.8 |
| Monocytes rest | 0.9 | 1.4 |
| Monocytes LPS | 66.4 | 81.2 |
| Macrophages rest | 16.2 | 9.7 |
| Macrophages LPS | 54.7 | 43.8 |
| HUVEC none | 9.3 | 12.6 |
| HUVEC starved | 14.4 | 25.2 |
| HUVEC IL-1beta | 15.6 | 18.9 |
| HUVEC IFN gamma | 12.9 | 16.7 |
| HUVEC TNF alpha + IFN gamma | 37.6 | 34.9 |
| HUVEC TNF alpha + IL4 | 31.4 | 31.4 |
| HUVEC IL-11 | 14.9 | 13.9 |
| Lung Microvascular EC none | 79.0 | 100.0 |
| Lung Microvascular EC TNFalpha + IL- | 100.0 | 97.9 |
| 1beta | | |
| Microvascular Dermal EC none | 49.7 | 48.3 |
| Microsvasular Dermal EC TNFalpha + IL- | 56.6 | 47.0 |
| 1beta | | |
| Bronchial epithelium TNFalpha + IL1beta | 78.5 | 90.1 |
| Small airway epithelium none | 31.0 | 32.5 |
| Small airway epithelium TNFalpha + IL- | 81.8 | 93.3 |
| 1beta | | |
| Coronery artery SMC rest | 17.2 | 28.5 |
| Coronery artery SMC TNFalpha + IL-1beta | 22.2 | 28.7 |
| Astrocytes rest | 80.1 | 55.1 |
| Astrocytes TNFalpha + IL-1beta | 82.9 | 66.4 |
| KU-812 (Basophil) rest | 2.6 | 1.9 |
| KU-812 (Basophil) PMA/ionomycin | 0.6 | 2.8 |
| CCD1106 (Keratinocytes) none | 70.7 | 82.4 |
| CCD1106 (Keratinocytes) TNFalpha + IL- | 77.4 | 72.7 |
| 1beta | | |
| Liver cirrhosis | 13.2 | 14.4 |
| NCI-H292 none | 57.8 | 54.0 |
| NCI-H292 IL-4 | 62.4 | 78.5 |
| NCI-H292 IL-9 | 61.6 | 79.6 |
| NCI-H292 IL-13 | 53.6 | 59.9 |
| NCI-H292 IFN gamma | 67.4 | 71.7 |
| HPAEC none | 21.5 | 21.3 |
| HPAEC TNF alpha + IL-1 beta | 37.6 | 45.4 |
| Lung fibroblast none | 22.4 | 29.3 |
| Lung fibroblast TNF alpha + IL-1 beta | 71.2 | 87.7 |
| Lung fibroblast IL-4 | 16.2 | 23.3 |
| Lung fibroblast IL-9 | 31.9 | 30.4 |
| Lung fibroblast IL-13 | 18.7 | 36.6 |
| Lung fibroblast IFN gamma | 23.0 | 29.7 |
| Dermal fibroblast CCD1070 rest | 15.9 | 27.2 |
| Dermal fibroblast CCD1070 TNF alpha | 15.9 | 20.6 |
| Dermal fibroblast CCD1070 IL-1 beta | 17.2 | 22.4 |
| Dermal fibroblast IFN gamma | 7.2 | 10.3 |
| Dermal fibroblast IL-4 | 7.2 | 8.0 |
| Dermal Fibroblasts rest | 4.3 | 6.3 |
| Neutrophils TNFa + LPS | 0.0 | 0.9 |
| Neutrophils rest | 0.2 | 1.0 |
| Colon | 7.0 | 5.8 |
| Lung | 23.3 | 23.3 |
| Thymus | 5.8 | 7.3 |
| Kidney | 23.2 | 33.2 |

Column A - Rel. Exp. (%) Ag3605, Run 169943454
Column B - Rel. Exp. (%) Ag3974, Run 170739806

TABLE BIF
general oncology screening panel_v_2.4

| Tissue Name | A |
|---|---|
| Colon cancer 1 | 11.6 |
| CC Margin (ODO3921) | 5.3 |
| Colon cancer 2 | 18.6 |
| Colon NAT 2 | 3.2 |
| Colon cancer 3 | 22.2 |
| Colon NAT 3 | 7.7 |
| Colon malignant cancer 4 | 23.5 |
| Colon NAT 4 | 2.0 |
| Lung cancer 1 | 57.0 |
| Lung NAT 1 | 3.1 |
| Lung cancer 2 | 100.0 |
| Lung NAT 2 | 3.9 |
| Squamous cell carcinoma 3 | 41.2 |
| Lung NAT 3 | 1.7 |
| Metastatic melanoma 1 | 5.4 |
| Melanoma 2 | 3.2 |
| Melanoma 3 | 3.7 |
| Metastatic melanoma 4 | 12.5 |
| Metastatic melanoma 5 | 7.9 |
| Bladder cancer 1 | 0.5 |
| Bladder NAT 1 | 0.0 |
| Bladder cancer 2 | 6.0 |
| Bladder NAT 2 | 0.2 |
| Bladder NAT 3 | 1.7 |
| Bladder NAT 4 | 1.8 |
| Prostate adenocarcinoma 1 | 2.4 |
| Prostate adenocarcinoma 2 | 2.6 |
| Prostate adenocarcinoma 3 | 5.2 |
| Prostate adenocarcinoma 4 | 21.9 |
| Prostate NAT 5 | 5.4 |
| Prostate adenocarcinoma 6 | 4.4 |
| Prostate adenocarcinoma 7 | 3.8 |
| Prostate adenocarcinoma 8 | 1.8 |
| Prostate adenocarcinoma 9 | 10.7 |
| Prostate NAT 10 | 0.7 |
| Kidney cancer 1 | 38.7 |
| Kidney NAT 1 | 20.6 |
| Kidney cancer 2 | 66.4 |
| Kidney NAT 2 | 39.8 |
| Kidney cancer 3 | 64.6 |
| Kidney NAT 3 | 19.6 |
| Kidney cancer 4 | 16.4 |
| Kidney NAT 4 | 15.6 |

Column A - Rel. Exp. (%) Ag3605, Run 260268655

General_screening_panel_v1.4 Summary: Ag3605/Ag3974 The expression of this gene was highest in a sample derived from a breast cancer cell line (T47D) (CTs=22.5-25.3). In addition, there was substantial expression in other samples derived from breast cancer cell lines, ovarian cancer cell lines, kidney cancer cell lines, lung cancer cell lines, colon cancer cell lines and brain cancer cell lines. Thus, the expression of this gene is useful as a marker to distinguish T47D cells from other samples in the panel. Therapeutic modulation of this gene or its protein product through the use of small molecule drugs, protein therapeutics, or antibodies is of benefit in the treatment of breast, ovarian, kidney, lung, colon or brain cancer.

Among metabolic tissues, this gene had low-to-moderate levels of expression in adrenal, pituitary, adult and fetal heart, adult and fetal liver, adult and fetal skeletal muscle, and adipose. This gene product had high levels of expression (CT values=27) in pancreas and thyroid. Thus, this gene product is important for the pathogenesis, diagnosis, and/or treatment of metabolic and endocrine diseases, including obesity, Types 1 and 2 diabetes and thyroidopathies. Decreased glomerular expression of agrin has been observed in diabetic nephropathy and supports this use of this gene or gene product. (Yard B A, Exp Nephrol 2001;9(3):214-22 ).

In addition, this gene was expressed at moderate levels in all regions of the central nervous system examined, including amygdala, hippocampus, substantia nigra, thalamus, cerebellum, cerebral cortex, and spinal cord. This gene encodes a protein with homology to agrin, a neuronal aggregating factor that induces the aggregation of acetylcholine receptors and other postsynaptic proteins on muscle fibers and is crucial for the formation of the neuromuscular junction. Agrin also plays an important role in defining neuronal responses to excitatory neurotransmitters both in vitro and in vivo (Hilgenberg L G, Mol Cell Neurosci 2002 January; 19(1):97-110 and Bixby J L, J Neurobiol 2002 February 5;50(2):164-79). The gene expression in the central nervous system is consistent the use of the protein and its function as an agrin. This gene plays a role in central nervous system disorders such as Alzheimer's disease, Parkinson's disease, epilepsy, multiple sclerosis, schizophrenia and depression.

Agrin has also been implicated in the formation of senile plaques in Alzheimer's disease and in the acetylcholine synapse/neuromuscular junction (van Horssen J, Acta Neuropathol (Berl) 2001 December;102(6):604-14). In addition, an agrin minigene rescued dystrophic symptoms in a mouse model of muscular dystrophy (Moll J, Nature. 2001 September 20;413(6853):302-7). Therefore, this gene or gene product is useful as a treatment or cure for congenital muscular dystrophies. Furthermore, this gene product is also an excellent drug target in AD or in any disease involving the neuromuscular junction or the acetylcholine system.

Panel 2.2 Summary: Ag3605 Expression of this gene was highest in a sample of normal kidney (CT=27.4). In addition, expression of this gene was upregulated in a number of ovarian and renal cancers when compared to the matched control margins.

Thus, expression of this gene is useful as a marker for ovarian and renal carcinoma. Furthermore, therapeutic modulation of the activity of this gene or its protein product, using small molecule drugs, antibodies or protein therapeutics, is of benefit in the treatment of renal and ovarian cancer.

Panel 4.1D Summary: Ag3605/Ag3974 Highest expression of this gene was highest in lung microvascular endothelial cells (CTs=27.3-28.5), microvascular dermal endothelial cells, mucoepidermoid cell line NCI-H292, astrocytes, and keratinocytes.

This gene encodes a protein with homology to agrin. Agrin, an aggregating protein crucial for formation of the neuromuscular junction, is important for T cell signaling in the immune system (Khan AA, Science 2001 June 1;292(5522):1681-6). In addition, agrin has been identified as a potential disease target for autoimmune disorders at the neuromuscularjunction, including multiple sclerosis (Liyanage Y, Muscle Nerve 2002 January;25(1):4-16). Therefore, small molecule drug, antibody or protein therapeutics that target this gene or the protein encoded by the gene is useful for the reduction or inhibition of inflammation in asthma, emphysema, allergy, psoriasis, muscular dystrophy and multiple sclerosis.

general oncology screening panel_V_{—}2.4 Summary: Ag3605 Highest expression of this gene was seen in a lung cancer sample (CT=26.8). In addition, higher levels of expression were seen in lung and kidney cancers when compared to expression in normal adjacent tissue. Thus, expression of this gene is useful as a marker of lung and kidney cancers. Therapeutic modulation of the expression or function of this gene or gene product is useful in the treatment of lung and kidney cancers.

BJ. CG9638401: Novel Plasma Membrane Protein

Expression of gene CG96384-01 was assessed using the primer-probe set Ag4093, described in Table BJA. Results of the RTQ-PCR run are shown in Table BJB.

TABLE BJA
Probe Name Ag4093

| | | | | |
|---|---|---|---|---|
| | +HL,6 | | | SEQ |
| | | | Start | ID |
| Primers | Sequences | Length | Position | No |

| | | | | |
|---|---|---|---|---|
| Forward | 5′-agctggagcacaggagagat-3′ | 20 | 158 | 1597 |
| Probe | TET-5′-ggaagctctcctttgacactcgttcc-3′- | 26 | 197 | 1598 |
| | TAMRA | | | |
| Reverse | 5′-acccatggtcttccagaaag-3′ | 20 | 231 | 1599 |

TABLE BJB
Panel 4.1D

| Tissue Name | A |
|---|---|
| Secondary Th1 act | 55.1 |
| Secondary Th2 act | 87.7 |
| Secondary Tr1 act | 82.9 |
| Secondary Th1 rest | 30.4 |
| Secondary Th2 rest | 43.2 |
| Secondary Tr1 rest | 38.7 |
| Primary Th1 act | 57.4 |
| Primary Th2 act | 100.0 |
| Primary Tr1 act | 62.0 |
| Primary Th1 rest | 28.3 |
| Primary Th2 rest | 21.5 |
| Primary Tr1 rest | 42.0 |
| CD45RA CD4 lymphocyte act | 40.9 |
| CD45RO CD4 lymphocyte act | 84.1 |
| CD8 lymphocyte act | 70.2 |
| Secondary CD8 lymphocyte rest | 56.6 |
| Secondary CD8 lymphocyte act | 26.6 |
| CD4 lymphocyte none | 36.1 |
| 2ry Th1/Th2/Tr1 anti-CD95 CH11 | 67.4 |
| LAK cells rest | 38.2 |
| LAK cells IL-2 | 46.0 |
| LAK cells IL-2 + IL-12 | 42.3 |
| LAK cells IL-2 + IFN gamma | 25.7 |
| LAK cells IL-2 + IL-18 | 35.4 |
| LAK cells PMA/ionomycin | 47.0 |
| NK Cells IL-2 rest | 74.7 |
| Two Way MLR 3 day | 36.3 |
| Two Way MLR 5 day | 19.6 |
| Two Way MLR 7 day | 58.6 |
| PBMC rest | 26.1 |
| PBMC PWM | 10.9 |
| PBMC PHA-L | 42.9 |
| Ramos (B cell) none | 18.9 |
| Ramos (B cell) ionomycin | 6.1 |
| B lymphocytes PWM | 27.7 |
| B lymphocytes CD40L and IL-4 | 48.0 |
| EOL-1 dbcAMP | 21.2 |
| EOL-1 dbcAMP PMA/ionomycin | 31.4 |
| Dendritic cells none | 17.4 |
| Dendritic cells LPS | 10.7 |
| Dendritic cells anti-CD40 | 16.7 |
| Monocytes rest | 29.5 |
| Monocytes LPS | 28.3 |
| Macrophages rest | 13.9 |
| Macrophages LPS | 7.6 |
| HUVEC none | 14.8 |
| HUVEC starved | 36.6 |
| HUVEC IL-1beta | 18.8 |
| HUVEC IFN gamma | 23.3 |
| HUVEC TNF alpha + IFN gamma | 11.3 |
| HUVEC TNF alpha + IL4 | 20.7 |
| HUVEC IL-11 | 12.2 |
| Lung Microvascular EC none | 21.0 |
| Lung Microvascular EC TNFalpha + IL-1beta | 15.3 |
| Microvascular Dermal EC none | 12.6 |
| Microsvasular Dermal EC TNFalpha + IL-1beta | 15.8 |
| Bronchial epithelium TNFalpha + IL1beta | 16.8 |
| Small airway epithelium none | 9.3 |
| Small airway epithelium TNFalpha + IL-1beta | 14.2 |
| Coronery artery SMC rest | 8.9 |
| Coronery artery SMC TNFalpha + IL-1 beta | 7.3 |
| Astrocytes rest | 9.8 |
| Astrocytes TNFalpha + IL-1beta | 8.2 |
| KU-812 (Basophil) rest | 11.4 |
| KU-812 (Basophil) PMA/ionomycin | 18.3 |
| CCD1106 (Keratinocytes) none | 13.2 |
| CCD1106 (Keratinocytes) TNFalpha + IL-1beta | 17.3 |
| Liver cirrhosis | 9.7 |
| NCI-H292 none | 15.8 |
| NCI-H292 IL-4 | 32.5 |
| NCI-H292 IL-9 | 33.2 |
| NCI-H292 IL-13 | 45.1 |
| NCI-H292 IFN gamma | 12.8 |
| HPAEC none | 24.3 |
| HPAEC TNF alpha + IL-1 beta | 36.6 |
| Lung fibroblast none | 18.7 |
| Lung fibroblast TNF alpha + IL-1 beta | 2.9 |
| Lung fibroblast IL-4 | 6.4 |
| Lung fibroblast IL-9 | 17.4 |
| Lung fibroblast IL-13 | 15.5 |
| Lung fibroblast IFN gamma | 14.9 |
| Dermal fibroblast CCD1070 rest | 17.9 |
| Dermal fibroblast CCD1070 TNF alpha | 67.8 |
| Dermal fibroblast CCD1070 IL-1 beta | 15.2 |
| Dermal fibroblast IFN gamma | 9.7 |
| Dermal fibroblast IL-4 | 44.1 |
| Dermal Fibroblasts rest | 16.6 |
| Neutrophils TNFa + LPS | 2.4 |
| Neutrophils rest | 19.6 |
| Colon | 8.1 |
| Lung | 8.7 |
| Thymus | 84.1 |
| Kidney | 55.9 |

Column A - Rel. Exp. (%) Ag4093, Run 172383903

Panel 4.1D Summary: Ag4093 This gene was widely expressed in this panel, with highest expression in primary activated Th2 cells (CT=32.7). This gene was also expressed. at high to moderate levels in a wide range of cell types of significance in the immune response in health and disease. These cells include members of the T-cell, B-cell, endothelial cell, monocyte, and peripheral blood mononuclear cell families, as well as dermal fibroblasts and normal tissues represented from thymus and kidney. This pattern of expression shows that this gene product is involved in homeostatic processes for these and other cell types and tissues. Therefore, modulation of the gene or gene product with a functional therapeutic is useful in the alteration of functions associated with these cell types and in the treatment of the symptoms of patients suffering from autoimmune and inflammatory diseases such as asthma, allergies, inflammatory bowel disease, lupus erythematosus, psoriasis, rheumatoid arthritis, and osteoarthritis.

BK. CG98011-01: Insulin-like Growth Factor Binding Protein

Expression of gene CG9801 1-01 was assessed using the primer-probe set Ag2120, described in Table BKA. Results of the RTQ-PCR runs are shown in Tables BKB, BKC, BKD and BKE.

TABLE BKA
Probe Name Ag2120

| | | | | |
|---|---|---|---|---|
| | +HL,6 | | | SEQ |
| | | | Start | ID |
| Primers | Sequences | Length | Position | No |

| | | | | |
|---|---|---|---|---|
| Forward | 5′-gctgattgcaagaagatgtttc-3′ | 22 | 103 | 1600 |
| Probe | TET-5′-ttttgtcagccctgatttcttcgaca-3′- | 26 | 140 | 1601 |
| Reverse | 5′-ccgatatgtcagaatctgcatt-3′ | 22 | 166 | 1602 |

TABLE BKB
Panel 1.3D

| Tissue Name | A | B |
|---|---|---|
| Liver adenocarcinoma | 0.5 | 0.1 |
| Pancreas | 0.3 | 0.2 |
| Pancreatic ca. CAPAN 2 | 1.0 | 1.1 |
| Adrenal gland | 12.8 | 10.8 |
| Thyroid | 2.1 | 1.3 |
| Salivary gland | 0.6 | 1.1 |
| Pituitary gland | 0.6 | 0.3 |
| Brain (fetal) | 7.9 | 8.8 |
| Brain (whole) | 26.8 | 23.8 |
| Brain (amygdala) | 27.5 | 17.8 |
| Brain (cerebellum) | 17.4 | 19.2 |
| Brain (hippocampus) | 64.6 | 58.6 |
| Brain (substantia nigra) | 2.2 | 3.5 |
| Brain (thalamus) | 3.6 | 3.0 |
| Cerebral Cortex | 100.0 | 100.0 |
| Spinal cord | 0.5 | 1.2 |
| glio/astro U87-MG | 0.8 | 0.5 |
| glio/astro U-118-MG | 3.2 | 4.7 |
| astrocytoma SW1783 | 0.7 | 0.8 |
| neuro*; met SK-N-AS | 0.4 | 0.2 |
| astrocytoma SF-539 | 0.4 | 0.3 |
| astrocytoma SNB-75 | 0.5 | 0.2 |
| glioma SNB-19 | 0.4 | 1.2 |
| glioma U251 | 1.6 | 1.3 |
| glioma SF-295 | 1.9 | 0.8 |
| Heart (Fetal) | 3.3 | 2.2 |
| Heart | 0.3 | 0.6 |
| Skeletal muscle (Fetal) | 6.8 | 9.3 |
| Skeletal muscle | 3.4 | 4.0 |
| Bone marrow | 0.9 | 1.6 |
| Thymus | 0.9 | 0.5 |
| Spleen | 0.4 | 0.1 |
| Lymph node | 1.2 | 0.6 |
| Colorectal | 1.6 | 1.1 |
| Stomach | 1.0 | 0.2 |
| Small intestine | 0.2 | 2.0 |
| Colon ca. SW480 | 0.8 | 1.6 |
| Colon ca.* SW620 (SW480 met) | 0.0 | 1.4 |
| Colon ca. HT29 | 0.2 | 0.5 |
| Colon ca. HCT-116 | 0.3 | 0.2 |
| Colon ca. CaCo-2 | 0.6 | 0.0 |
| CC Well to Mod Diff (ODO3866) | 1.5 | 0.5 |
| Colon ca. HCC-2998 | 0.6 | 0.3 |
| Gastric ca. (liver met) NCI-N87 | 0.5 | 1.2 |
| Bladder | 1.8 | 1.0 |
| Trachea | 0.6 | 1.3 |
| Kidney | 1.3 | 2.5 |
| Kidney (fetal) | 1.7 | 4.0 |
| Renal ca. 786-0 | 3.8 | 3.7 |
| Renal ca. A498 | 2.6 | 3.0 |
| Renal ca. RXF 393 | 2.2 | 2.3 |
| Renal ca. ACHN | 1.1 | 0.8 |
| Renal ca. UO-31 | 0.5 | 1.2 |
| Renal ca. TK-10 | 3.7 | 3.6 |
| Liver | 0.8 | 0.0 |
| Liver (fetal) | 0.2 | 0.1 |
| Liver ca. (hepatoblast) HepG2 | 0.7 | 0.8 |
| Lung | 0.2 | 0.7 |
| Lung (fetal) | 0.4 | 0.6 |
| Lung ca. (small cell) LX-1 | 1.2 | 0.1 |
| Lung ca. (small cell) NCI-H69 | 44.8 | 40.9 |
| Lung ca. (s. cell var.) SHP-77 | 5.6 | 5.2 |
| Lung ca. (large cell)NCI-H460 | 54.3 | 74.2 |
| Lung ca. (non-sm. cell) A549 | 0.5 | 0.0 |
| Lung ca. (non-s. cell) NCI-H23 | 1.5 | 2.0 |
| Lung ca. (non-s. cell) HOP-62 | 7.0 | 5.3 |
| Lung ca. (non-s. cl) NCI-H522 | 1.1 | 0.0 |
| Lung ca. (squam.) SW 900 | 0.3 | 0.8 |
| Lung ca. (squam.) NCI-H596 | 8.4 | 11.1 |
| Mammary gland | 2.4 | 2.0 |
| Breast ca.* (pl. ef) MCF-7 | 1.0 | 0.7 |
| Breast ca.* (pl. ef) MDA-MB-231 | 0.0 | 0.0 |
| Breast ca.* (pl. ef) T47D | 17.3 | 18.7 |
| Breast ca. BT-549 | 4.2 | 4.7 |
| Breast ca. MDA-N | 9.3 | 9.9 |
| Ovary | 12.4 | 9.9 |
| Ovarian ca. OVCAR-3 | 0.1 | 0.0 |
| Ovarian ca. OVCAR-4 | 0.5 | 2.7 |
| Ovarian ca. OVCAR-5 | 0.9 | 0.9 |
| Ovarian ca. OVCAR-8 | 2.1 | 4.0 |
| Ovarian ca. IGROV-1 | 0.4 | 0.3 |
| Ovarian ca. (ascites) SK-OV-3 | 0.8 | 0.1 |
| Uterus | 1.2 | 0.7 |
| Placenta | 1.8 | 0.1 |
| Prostate | 0.5 | 0.6 |
| Prostate ca.* (bone met) PC-3 | 3.1 | 2.2 |
| Testis | 1.2 | 1.3 |
| Melanoma Hs688(A).T | 0.8 | 0.9 |
| Melanoma* (met) Hs688(B).T | 1.2 | 2.7 |
| Melanoma UACC-62 | 0.7 | 0.7 |
| Melanoma M14 | 4.0 | 5.5 |
| Melanoma LOX IMVI | 1.6 | 0.3 |
| Melanoma* (met) SK-MEL-5 | 4.2 | 3.7 |
| Adipose | 3.1 | 1.9 |

Column A - Rel. Exp. (%) Ag2120, Run 149975625
Column B - Rel. Exp. (%) Ag2120, Run 150253839

TABLE BKC
Panel 2D

| Tissue Name | A | B |
|---|---|---|
| Normal Colon | 7.3 | 7.4 |
| CC Well to Mod Diff (ODO3866) | 4.3 | 7.9 |
| CC Margin (ODO3866) | 1.7 | 2.9 |
| CC Gr.2 rectosigmoid (ODO3868) | 2.3 | 1.5 |
| CC Margin (ODO3868) | 1.7 | 3.8 |
| CC Mod Diff (ODO3920) | 0.2 | 0.9 |
| CC Margin (ODO3920) | 0.1 | 2.5 |
| CC Gr.2 ascend colon (ODO3921) | 0.5 | 0.4 |
| CC Margin (ODO3921) | 5.8 | 6.4 |
| CC from Partial Hepatectomy | 6.1 | 8.7 |
| (ODO4309) Mets | | |
| Liver Margin (ODO4309) | 4.0 | 2.2 |
| Colon mets to lung (OD04451-01) | 0.0 | 0.3 |
| Lung Margin (OD04451-02) | 2.9 | 3.9 |
| Normal Prostate 6546-1 | 3.0 | 7.8 |
| Prostate Cancer (OD04410) | 8.5 | 9.0 |
| Prostate Margin (OD04410) | 23.5 | 21.5 |
| Prostate Cancer (OD04720-01) | 7.3 | 7.1 |
| Prostate Margin (OD04720-02) | 1.7 | 11.3 |
| Normal Lung | 9.2 | 7.2 |
| Lung Met to Muscle (ODO4286) | 0.7 | 0.7 |
| Muscle Margin (ODO4286) | 11.3 | 12.5 |
| Lung Malignant Cancer (OD03126) | 7.9 | 4.7 |
| Lung Margin (OD03126) | 6.4 | 7.5 |
| Lung Cancer (OD04404) | 3.0 | 3.8 |
| Lung Margin (OD04404) | 2.6 | 2.8 |
| Lung Cancer (OD04565) | 1.8 | 3.4 |
| Lung Margin (OD04565) | 3.6 | 2.8 |
| Lung Cancer (OD04237-01) | 22.5 | 17.3 |
| Lung Margin (OD04237-02) | 3.5 | 4.5 |
| Ocular Mel Met to Liver (ODO4310) | 2.1 | 4.1 |
| Liver Margin (ODO4310) | 1.2 | 1.2 |
| Melanoma Metastasis | 2.5 | 1.7 |
| Lung Margin (OD04321) | 5.3 | 3.3 |
| Normal Kidney | 93.3 | 100.0 |
| Kidney Ca, Nuclear grade 2 (OD04338) | 55.9 | 92.7 |
| Kidney Margin (OD04338) | 37.9 | 36.6 |
| Kidney Ca Nuclear grade 1/2 (OD04339) | 67.4 | 76.3 |
| Kidney Margin (OD04339) | 25.3 | 33.2 |
| Kidney Ca, Clear cell type (OD04340) | 52.5 | 43.5 |
| Kidney Margin (OD04340) | 0.0 | 35.6 |
| Kidney Ca, Nuclear grade 3 (OD04348) | 5.0 | 7.6 |
| Kidney Margin (OD04348) | 20.7 | 26.6 |
| Kidney Cancer (OD04622-01) | 9.3 | 7.3 |
| Kidney Margin (OD04622-03) | 7.1 | 10.2 |
| Kidney Cancer (OD04450-01) | 27.0 | 29.3 |
| Kidney Margin (OD04450-03) | 34.2 | 33.7 |
| Kidney Cancer 8120607 | 4.1 | 3.3 |
| Kidney Margin 8120608 | 9.2 | 12.8 |
| Kidney Cancer 8120613 | 2.2 | 3.3 |
| Kidney Margin 8120614 | 7.3 | 14.9 |
| Kidney Cancer 9010320 | 22.4 | 26.4 |
| Kidney Margin 9010321 | 18.3 | 26.8 |
| Normal Uterus | 9.6 | 8.6 |
| Uterine Cancer 064011 | 2.7 | 2.6 |
| Normal Thyroid | 5.1 | 5.3 |
| Thyroid Cancer | 39.2 | 44.1 |
| Thyroid Cancer A302152 | 30.8 | 24.5 |
| Thyroid Margin A302153 | 3.3 | 3.6 |
| Normal Breast | 5.0 | 4.5 |
| Breast Cancer | 0.8 | 1.7 |
| Breast Cancer (OD04590-01) | 9.7 | 7.6 |
| Breast Cancer Mets (OD04590-03) | 26.1 | 33.4 |
| Breast Cancer Metastasis | 3.4 | 4.1 |
| Breast Cancer | 3.3 | 4.4 |
| Breast Cancer | 7.2 | 8.3 |
| Breast Cancer 9100266 | 3.1 | 3.7 |
| Breast Margin 9100265 | 3.3 | 3.9 |
| Breast Cancer A209073 | 8.2 | 8.9 |
| Breast Margin A209073 | 16.2 | 11.6 |
| Normal Liver | 1.4 | 0.9 |
| Liver Cancer | 1.8 | 5.1 |
| Liver Cancer 1025 | 1.3 | 2.0 |
| Liver Cancer 1026 | 3.4 | 2.4 |
| Liver Cancer 6004-T | 1.6 | 0.4 |
| Liver Tissue 6004-N | 1.3 | 1.0 |
| Liver Cancer 6005-T | 0.0 | 6.5 |
| Liver Tissue 6005-N | 1.6 | 0.3 |
| Normal Bladder | 3.4 | 9.5 |
| Bladder Cancer | 10.7 | 6.5 |
| Bladder Cancer | 1.8 | 2.1 |
| Bladder Cancer (OD04718-01) | 1.7 | 5.2 |
| Bladder Normal Adjacent (OD04718-03) | 4.2 | 6.3 |
| Normal Ovary | 9.5 | 7.4 |
| Ovarian Cancer | 100.0 | 95.3 |
| Ovarian Cancer (OD04768-07) | 4.6 | 7.6 |
| Ovary Margin (OD04768-08) | 7.3 | 5.7 |
| Normal Stomach | 1.7 | 3.8 |
| Gastric Cancer 9060358 | 2.1 | 0.5 |
| Stomach Margin 9060359 | 1.6 | 3.1 |
| Gastric Cancer 9060395 | 3.3 | 3.1 |
| Stomach Margin 9060394 | 2.2 | 3.3 |
| Gastric Cancer 9060397 | 11.0 | 14.5 |
| Stomach Margin 9060396 | 2.8 | 4.3 |
| Gastric Cancer 064005 | 2.2 | 6.5 |

Column A - Rel. Exp. (%) Ag2120, Run 149975659
Column B - Rel. Exp. (%) Ag2120, Run 150010138

TABLE BKD
Panel 4D

| Tissue Name | A |
|---|---|
| Secondary Th1 act | 0.4 |
| Secondary Th2 act | 0.8 |
| Secondary Tr1 act | 3.1 |
| Secondary Th1 rest | 3.4 |
| Secondary Th2 rest | 1.5 |
| Secondary Tr1 rest | 1.5 |
| Primary Th1 act | 0.3 |
| Primary Th2 act | 0.8 |
| Primary Tr1 act | 0.4 |
| Primary Th1 rest | 3.1 |
| Primary Th2 rest | 5.5 |
| Primary Tr1 rest | 0.6 |
| CD45RA CD4 lymphocyte act | 1.3 |
| CD45RO CD4 lymphocyte act | 2.4 |
| CD8 lymphocyte act | 2.2 |
| Secondary CD8 lymphocyte rest | 4.8 |
| Secondary CD8 lymphocyte act | 2.4 |
| CD4 lymphocyte none | 0.9 |
| 2ry Th1/Th2/Tr1 anti-CD95 CH11 | 0.7 |
| LAK cells rest | 7.2 |
| LAK cells IL-2 | 0.4 |
| LAK cells IL-2 + IL-12 | 2.8 |
| LAK cells IL-2 + IFN gamma | 7.7 |
| LAK cells IL-2 + IL-18 | 4.1 |
| LAK cells PMA/ionomycin | 3.2 |
| NK Cells IL-2 rest | 2.3 |
| Two Way MLR 3 day | 2.7 |
| Two Way MLR 5 day | 4.0 |
| Two Way MLR 7 day | 5.2 |
| PBMC rest | 6.4 |
| PBMC PWM | 5.2 |
| PBMC PHA-L | 1.8 |
| Ramos (B cell) none | 3.9 |
| Ramos (B cell) ionomycin | 1.6 |
| B lymphocytes PWM | 0.0 |
| B lymphocytes CD40L and IL-4 | 3.2 |
| EOL-1 dbcAMP | 67.4 |
| EOL-1 dbcAMP PMA/ionomycin | 56.6 |
| Dendritic cells none | 6.0 |
| Dendritic cells LPS | 15.5 |
| Dendritic cells anti-CD40 | 13.5 |
| Monocytes rest | 46.0 |
| Monocytes LPS | 4.9 |
| Macrophages rest | 47.3 |
| Macrophages LPS | 24.8 |
| HUVEC none | 10.2 |
| HUVEC starved | 23.8 |
| HUVEC IL-1beta | 11.2 |
| HUVEC IFN gamma | 10.1 |
| HUVEC TNF alpha + IFN gamma | 4.1 |
| HUVEC TNF alpha + IL4 | 13.2 |
| HUVEC IL-11 | 8.1 |
| Lung Microvascular EC none | 3.2 |
| Lung Microvascular EC TNFalpha + IL-1beta | 2.4 |
| Microvascular Dermal EC none | 0.0 |
| Microsvasular Dermal EC TNFalpha + IL-1beta | 0.0 |
| Bronchial epithelium TNFalpha + IL1beta | 3.1 |
| Small airway epithelium none | 5.4 |
| Small airway epithelium TNFalpha + IL-1beta | 3.0 |
| Coronery artery SMC rest | 35.6 |
| Coronery artery SMC TNFalpha + IL-1beta | 39.0 |
| Astrocytes rest | 1.1 |
| Astrocytes TNFalpha + IL-1beta | 0.9 |
| KU-812 (Basophil) rest | 0.5 |
| KU-812 (Basophil) PMA/ionomycin | 2.0 |
| CCD1106 (Keratinocytes) none | 0.9 |
| CCD1106 (Keratinocytes) TNFalpha + IL-1beta | 2.6 |
| Liver cirrhosis | 5.5 |
| Lupus kidney | 9.3 |
| NCI-H292 none | 2.0 |
| NCI-H292 IL-4 | 5.3 |
| NCI-H292 IL-9 | 3.8 |
| NCI-H292 IL-13 | 1.3 |
| NCI-H292 IFN gamma | 3.4 |
| HPAEC none | 9.0 |
| HPAEC TNF alpha + IL-1 beta | 15.4 |
| Lung fibroblast none | 1.7 |
| Lung fibroblast TNF alpha + IL-1 beta | 0.0 |
| Lung fibroblast IL-4 | 2.8 |
| Lung fibroblast IL-9 | 1.6 |
| Lung fibroblast IL-13 | 1.0 |
| Lung fibroblast IFN gamma | 1.4 |
| Dermal fibroblast CCD1070 rest | 0.6 |
| Dermal fibroblast CCD1070 TNF alpha | 2.3 |
| Dermal fibroblast CCD1070 IL-1 beta | 5.4 |
| Dermal fibroblast IFN gamma | 0.5 |
| Dermal fibroblast IL-4 | 3.0 |
| IBD Colitis 2 | 5.8 |
| IBD Crohn's | 4.2 |
| Colon | 5.8 |
| Lung | 7.0 |
| Thymus | 100.0 |
| Kidney | 18.2 |

Column A - Rel. Exp. (%) Ag2120, Run 149975626

TABLE BKE
Panel CNS_1

| Tissue Name | A |
|---|---|
| BA4 Control | 46.3 |
| BA4 Control2 | 42.9 |
| BA4 Alzheimer's2 | 5.1 |
| BA4 Parkinson's | 38.4 |
| BA4 Parkinson's2 | 100.0 |
| BA4 Huntington's | 53.6 |
| BA4 Huntington's2 | 4.3 |
| BA4 PSP | 4.5 |
| BA4 PSP2 | 14.1 |
| BA4 Depression | 11.8 |
| BA4 Depression2 | 3.5 |
| BA7 Control | 45.4 |
| BA7 Control2 | 37.9 |
| BA7 Alzheimer's2 | 8.5 |
| BA7 Parkinson's | 11.7 |
| BA7 Parkinson's2 | 54.3 |
| BA7 Huntington's | 41.2 |
| BA7 Huntington's2 | 34.2 |
| BA7 PSP | 36.1 |
| BA7 PSP2 | 21.2 |
| BA7 Depression | 4.5 |
| BA9 Control | 18.0 |
| BA9 Control2 | 76.3 |
| BA9 Alzheimer's | 8.0 |
| BA9 Alzheimer's2 | 4.6 |
| BA9 Parkinson's | 28.7 |
| BA9 Parkinson's2 | 55.5 |
| BA9 Huntington's | 34.2 |
| BA9 Huntington's2 | 4.7 |
| BA9 PSP | 11.6 |
| BA9 PSP2 | 3.8 |
| BA9 Depression | 5.0 |
| BA9 Depression2 | 3.2 |
| BA17 Control | 36.9 |
| BA17 Control2 | 62.0 |
| BA17 Alzheimer's2 | 2.9 |
| BA17 Parkinson's | 13.4 |
| BA17 Parkinson's2 | 61.1 |
| BA17 Huntington's | 27.9 |
| BA17 Huntington's2 | 8.4 |
| BA17 Depression | 2.3 |
| BA17 Depression2 | 10.1 |
| BA17 PSP | 20.0 |
| BA17 PSP2 | 10.5 |
| Sub Nigra Control | 10.6 |
| Sub Nigra Control2 | 29.1 |
| Sub Nigra Alzheimer's2 | 5.8 |
| Sub Nigra Parkinson's2 | 20.6 |
| Sub Nigra Huntington's | 21.5 |
| Sub Nigra Huntington's2 | 14.7 |
| Sub Nigra PSP2 | 3.6 |
| Sub Nigra Depression | 3.2 |
| Sub Nigra Depression2 | 2.4 |
| Glob Palladus Control | 1.8 |
| Glob Palladus Control2 | 7.8 |
| Glob Palladus Alzheimer's | 6.4 |
| Glob Palladus Alzheimer's2 | 2.5 |
| Glob Palladus Parkinson's | 34.4 |
| Glob Palladus Parkinson's2 | 3.1 |
| Glob Palladus PSP | 5.2 |
| Glob Palladus PSP2 | 0.0 |
| Glob Palladus Depression | 3.7 |
| Temp Pole Control | 11.0 |
| Temp Pole Control2 | 40.1 |
| Temp Pole Alzheimer's | 2.7 |
| Temp Pole Alzheimer's2 | 3.4 |
| Temp Pole Parkinson's | 13.9 |
| Temp Pole Parkinson's2 | 20.7 |
| Temp Pole Huntington's | 27.5 |
| Temp Pole PSP | 2.5 |
| Temp Pole PSP2 | 3.7 |
| Temp Pole Depression2 | 1.8 |
| Cing Gyr Control | 70.7 |
| Cing Gyr Control2 | 23.8 |
| Cing Gyr Alzheimer's | 13.8 |
| Cing Gyr Alzheimer's2 | 7.7 |
| Cing Gyr Parkinson's | 13.2 |
| Cing Gyr Parkinson's2 | 23.0 |
| Cing Gyr Huntington's | 40.3 |
| Cing Gyr Huntington's2 | 12.9 |
| Cing Gyr PSP | 7.6 |
| Cing Gyr PSP2 | 5.6 |
| Cing Gyr Depression | 4.1 |
| Cing Gyr Depression2 | 4.6 |

Column A - Rel. Exp. (%) Ag2120, Run 171649389

Panel 1.3D Summary: Ag2120 Expression of this gene was highest in the cerebral cortex (CT value=29). Moderate expression was detected in all other regions of the brain except thalamus and substantia nigra, demonstrating that this gene is associated with normal brain homeostasis. This protein showed a brain-preferential expression, see write-up on Panel CNS.01 for discussion of utility. In addition, expression of this gene was down-regulated in CNS cancer cell lines. Over-expression of this gene was also detected in several lung cancer cell lines relative to normal control. Therefore, this gene is useful for the detection or treatment of CNS and lung cancers.

Panel 2D Summary: Ag2120 Expression of this gene in panel 2D revealed an association of expression in thyroid, breast and kidney cancers when compared to their respective normal adjacent tissues. Thus, therapeutic modulation of this gene with inhibitory monoclonal antibodies and/or small molecule therapeutics is useful in treatment of these diseases. In addition, the SC98428706_EXT gene might be useful as a marker for thyroid, breast and kidney cancers.

Panel 4D Summary: Ag 2120 This gene showed highest expression levels in the thymus. (CT value=31). The transcript was also expressed in eosinophils, monocytes, macrophages and coronary artery. It was down regulated in LPS-treated monocytes and to a lesser degree in LPS treated macrophages. Therefore, protein therapeutics (agonists or antagonists) designed against the protein encoded for by this transcript are useful for reducing the inflammatory process observed in asthma, emphysema, osteoarthritis and sepsis.

Panel CNS_{—}1 Summary: Ag2120 Insulin and insulin-like growth factors belong to a family of polypeptides essential for proper regulation of physiologic processes such as energy metabolism, cell proliferation, development, and differentiation. The insulin-like growth factors bind to IGF with high affinity and compete with the IGF receptor for IGF binding. Transgenic mice over expressing insulin-like growth factor binding proteins (IGFBPs) tend to show brain developmental abnormalities, suggesting a role for these proteins in neurodevelopment. Furthermore, treatment with glycosaminoglycans (which increases muscle re-innervation after motor neuron death) upregulates serum levels of both IGF and IGFBP. Thus, the novel IGFBP encoded by this gene is useful in the treatment of diseases such as ALS, multiple sclerosis, and peripheral nerve injury on the basis of its homology to other established IGFBPS. The expression profile of this gene shows that it is expressed preferentially in the brain, with highest levels in the cerebral cortex and hippocampus, two regions that are known to degenerate in Alzheimer's disease. Examination of the expression profile on Panel CNS.01 showed that most regions of both control and diseased brains express this protein; however the levels were decreased in the motor cortex in progressive supranuclear palsy and depression. Thus, this protein is of use in the treatment of Alzheimer's disease, progressive supranuclear palsy, and depression.

### Example D

### CG56008, NOV53

### DETAILED DESCRIPTION

The present invention is based in part on the discovery that reducing the amount of LIV-1 mRNA present in tumor derived cells results in strongly reduced cell growth. The expression of this gene in tumor-derived cells was first defined using RTQPCR analysis.

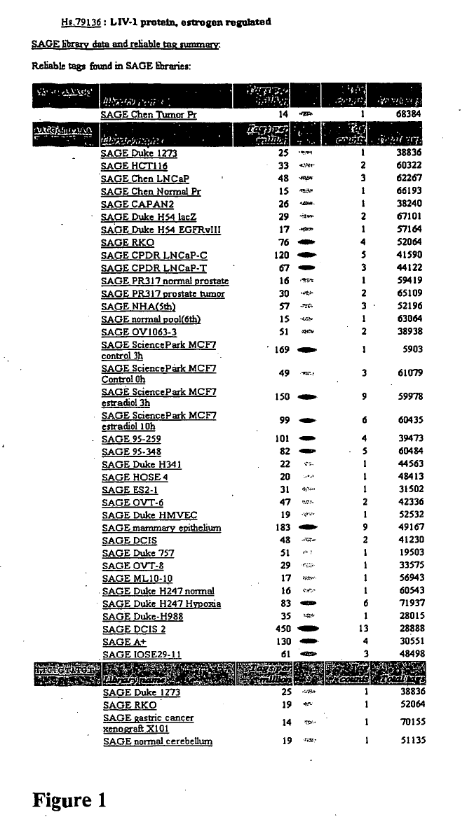

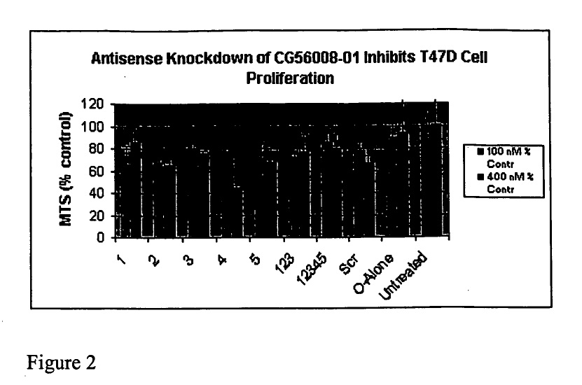

The RTQPCR analysis of panel 1.3d reveals that this gene has a restricted high expression in 5 breast cancer cell lines, with the highest expression in MCF-7 breast cancer cells. This gene is present in normal tissues at low levels (FIG. 1). The specific high expression in breast cancer cell lines indicates a role in breast cancer progression. This is further supported by the analysis of panel 2d that reveals how LIV-1 is upregulated specifically in breast cancers, comparing to the normal adjacent tissues (FIG. 2). Based upon its profile, the expression of this gene could be of use as a marker for the presence of breast cancer in a subject, specifically in a subject blood. In addition, down-regulation of the activity of this gene, through the use of antibodies or antisense oligonucleotide, might be of use in the treatment of breast cancer.

The expression profiles of this gene on our RTQPCR panels indicate that this gene may be involved in regulating cancer cell proliferation and oncogenesis. To confirm whether LIV-1 expression is required for cell growth, we used antisense technology to examine the effect of decreasing LIV-1 gene expression on breast cancer cell proliferation. This technology has been used to asses the biological/therapeutic effect of reducing (knockdown) the expression of a gene (see Tamm et al., Lancet 2001 August 11;358(9280):489-97 for a review of the current use of this technology in the biomedical field)

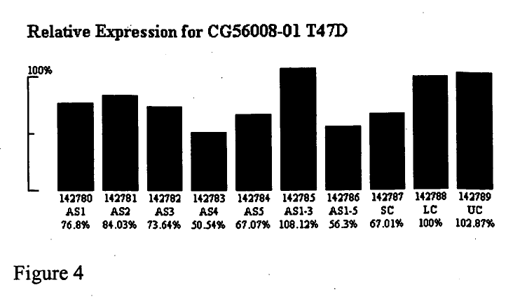

As shown in FIG. 4, transfection of LIV-1 antisense oligonucleotide AS4 at 400 nM concentration inhibited T47D breast carcinoma cell growth more than 50% (P=0.000189), whereas transfection of scramble antisense control had little effect on cell growth. We observed less inhibitory effect with LIV-1 antisense oligo AS4 transfection in Ovcar4 cells in which the expression of LIV-1 is much lower (P=0.138366) (FIG. 5). Knockdown of LIV-1 transcripts by antisense oligonucleotides was validated by RTQPCR. As shown in FIG. 6, transfection of antisense oligo AS4 resulted in 50% knockdown of LWV-1 expression at the mRNA level.

Therefore, our results suggested that LIV-1 expression is involved in cell proliferation. The inhibition of the activity of this gene through the use of antibodies or antisense oligonucleotides, mnight be of use in the treatment of breast cancer.

### Example D1

### Expression Analysis of LIV-1 in Various Tissues

RTOPCR Probe/Primer Information for LIV-1

RTQPCR Probe/Primers Set Ag2169

(SEQ ID NO: 1603)

Ag 2169 (F):
5′-CCCGAAAAGGCTTTATGTATTC-3′

(SEQ ID NO: 1604)

Ag 2169 (R):
5′-TGTCAGTAGCTTTGATGCATTG-3′

(SEQ ID NO: 1605)

Ag 2169 (P):
FAM-5′-CAGAAACACAAATGAAAATCCTCAGGA-3′-TAMRA

TABLE D1
RTQPCR results for Panel 1.3d.

| | Rel. Expr., % | Rel. Expr., % |
|---|---|---|
| | 1.3dtm3016f_{—} | 1.3Dtm3183f_{—} |
| Tissue Name | ag2169 | ag2169 |
| Liver adenocarcinoma | 1.8 | 2 |
| Pancreas | 1 | 0.4 |
| Pancreatic ca. CAPAN 2 | 1 | 1 |
| Adrenal gland | 0.8 | 0.6 |
| Thyroid | 2 | 0.9 |
| Salivary gland | 1.2 | 0.8 |
| Pituitary gland | 3.1 | 2.2 |
| Brain (fetal) | 2.2 | 1.7 |
| Brain (whole) | 2.6 | 2.1 |
| Brain (amygdala) | 2 | 1.1 |
| Brain (cerebellum) | 1.4 | 0.9 |
| Brain (hippocampus) | 6.1 | 4.4 |
| Brain (substantia nigra) | 0.5 | 0.8 |
| Brain (thalamus) | 2.5 | 2 |
| Cerebral Cortex | 2.8 | 3.1 |
| Spinal cord | 1.6 | 1.4 |
| glio/astro U87-MG | 1.2 | 0.8 |
| glio/astro U-118-MG | 12 | 9.3 |
| astro SW1783 | 2.8 | 3 |
| neuro; met SK-N-AS | 10.7 | 6.7 |
| astro SF-539 | 1.7 | 1.5 |
| astro SNB-75 | 2.8 | 3.8 |
| glio SNB-19 | 1 | 0.9 |
| glio U251 | 0.8 | 0.8 |
| glio SF-295 | 3.4 | 3 |
| Heart (fetal) | 0.4 | 0.5 |
| Heart | 0.2 | 0.1 |
| Fetal Skeletal | 1.2 | 1.4 |
| Skeletal muscle | 0.2 | 0.2 |
| Bone marrow | 0.4 | 0.2 |
| Thymus | 0.3 | 0.3 |
| Spleen | 1.1 | 0.8 |
| Lymph node | 0.8 | 0.5 |
| Colorectal | 0.3 | 0.2 |
| Stomach | 1.5 | 0.8 |
| Small intestine | 0.9 | 0.5 |
| Colon SW480 | 1.6 | 1.2 |
| Colon SW620(SW480 met) | 0.7 | 0.5 |
| Colon HT29 | 0.8 | 0.6 |
| Colon HCT-116 | 4.2 | 3.1 |
| Colon CaCo-2 | 0.9 | 1.1 |
| Colon Ca tissue(ODO3866) | 1.3 | 1.2 |
| Colon HCC-2998 | 2.1 | 1.6 |
| Gastric(liver met) NCI-N87 | 2 | 1.6 |
| Bladder | 1 | 0.6 |
| Trachea | 1.6 | 1.6 |
| Kidney | 0.5 | 0.5 |
| Kidney (fetal) | 1.1 | 0.8 |
| Renal 786-0 | 2.6 | 1.7 |
| Renal A498 | 4.2 | 3.2 |
| Renal RXF 393 | 1.2 | 0.8 |
| Renal ACHN | 2.6 | 2.7 |
| Renal UO-31 | 3.3 | 2.4 |
| Renal TK-10 | 2 | 1.5 |
| Liver | 0.1 | 0 |
| Liver (fetal) | 0.5 | 0.3 |
| Liver (hepatoblast) HepG2 | 1.5 | 1.3 |
| Lung | 0.8 | 0.6 |
| Lung (fetal) | 1.5 | 1.5 |
| Lung (small cell) LX-1 | 1 | 0.7 |
| Lung (small cell) NCI-H69 | 10 | 6.3 |
| Lung (s. cell var.) SHP-77 | 3.9 | 4.9 |
| Lung (large cell)NCI-H460 | 1.3 | 1.2 |
| Lung (non-sm. cell) A549 | 0.9 | 0.6 |
| Lung (non-s. cell) NCI-H23 | 5.4 | 0 |
| Lung (non-s. cell) HOP-62 | 1.8 | 2 |
| Lung (non-s. cl) NCI-H522 | 1.8 | 1.2 |
| Lung (squam.) SW 900 | 1.2 | 0.8 |
| Lung (squam.) NCI-H596 | 3.1 | 3 |
| Mammary gland | 11.7 | 10.4 |
| Breast (pl. ef) MCF-7 | 100 | 100 |
| Breast (pl.ef) MDA-MB-231 | 2.5 | 2.1 |
| Breast (pl.ef) T47D | 5.7 | 3.3 |
| Breast BT-549 | 4.5 | 3.6 |
| Breast MDA-N | 2.6 | 2.8 |
| Ovary | 2 | 1.3 |
| Ovarian OVCAR-3 | 2.2 | 2 |
| Ovarian OVCAR-4 | 0.3 | 0.2 |
| Ovarian OVCAR-5 | 0.6 | 0.5 |
| Ovarian OVCAR-8 | 1.6 | 0.9 |
| Ovarian IGROV-1 | 0.8 | 0.5 |
| Ovarian (ascites) SK-OV-3 | 4 | 3.2 |
| Uterus | 1.1 | 0.8 |
| Plancenta | 3.4 | 2.1 |
| Prostate | 5.5 | 4.6 |
| Prostate (bone met)PC-3 | 2 | 1.3 |
| Testis | 1.9 | 1.6 |
| Melanoma Hs688(A).T | 4.8 | 4.8 |
| Melanoma (met) Hs688(B).T | 6.2 | 5.2 |
| Melanoma UACC-62 | 0.3 | 0.3 |
| Melanoma M14 | 2.8 | 2.6 |
| Melanoma LOX IMVI | 0.6 | 0.4 |
| Melanoma (met) SK-MEL-5 | 7.1 | 5.1 |
| Adipose | 1.2 | 0.8 |

TABLE D2
RTQPCR results for Panel 2d.

| | Rel. Expr., % | Rel. Expr., % |
|---|---|---|
| Tissue Name | 2Dtm2969f_ag2169 | 2dtm3017f_ag2169 |
| Normal Colon | 3.2 | 3.1 |
| CCa 1 | 0.6 | 0.5 |
| CCa 1 Margin | 0.2 | 0.4 |
| CCa 2 | 0.1 | 0.2 |
| CCa 2 Margin | 0.2 | 0.1 |
| CCa 3 | 0.2 | 0.2 |
| CCa 3 Margin | 0.3 | 0.2 |
| CCa 4 | 1 | 1.1 |
| CCa 4 Margin | 0.3 | 0.4 |
| CCa 5 Metastasis | 1.6 | 1.8 |
| CCa 5 Margin (Liver) | 0.5 | 0.4 |
| CCa 6 Metastatsis | 0.2 | 0.2 |
| CCa 6 Margin (Lung) | 0.4 | 0.3 |
| Normal Prostate | 7.7 | 7.9 |
| PCa 1 | 15.1 | 18.7 |
| PCa 1 Margin | 7.4 | 7.9 |
| PCa 2 | 3.4 | 4 |
| PCa 2 Margin | 6.7 | 7.3 |
| Normal Lung | 1.4 | 1.6 |
| LCa 1 Metastasis | 1.4 | 1.5 |
| LCa 1 Margin (muscle) | 0.7 | 0.7 |
| LCa 2 | 1.7 | 2 |
| LCa 2 Margin | 1.1 | 1.3 |
| LCa 3 | 2 | 2.6 |
| LCa 3 Margin | 1 | 1.2 |
| LCa 4 | 1 | 1.1 |
| LCa 4 Margin | 0.5 | 0.5 |
| LCa 5 | 3.1 | 3.4 |
| LCa 5 Margin | 0.9 | 1 |
| Ocular Melanoma Metastasis | 3.7 | 4.1 |
| Liver Margin | 0.2 | 0.4 |
| Melanoma Metastasis | 3.5 | 4 |
| Lung Margin | 0.9 | 1.4 |
| Normal Kidney | 2.5 | 3.1 |
| RCC 1 | 2.8 | 2.8 |
| RCC 1 Margin | 1.8 | 1.9 |
| RCC 2 | 0.7 | 0.6 |
| RCC 2 Margin | 1.4 | 1.4 |
| RCC 3 | 2.5 | 3.1 |
| RCC 3 Margin | 1.8 | 1.9 |
| RCC 4 | 1 | 0.9 |
| RCC 4 Margin | 1.5 | 1.7 |
| RCC 5 | 0.9 | 0.9 |
| RCC 5 Margin | 0.2 | 0.1 |
| RCC 6 | 1.1 | 1 |
| RCC 6 Margin | 1.4 | 1.5 |
| RCC 7 | 0.5 | 0.4 |
| RCC 7 Margin | 0.3 | 0.2 |
| RCC 8 | 0.4 | 0.4 |
| RCC 8 Margin | 0.2 | 0.2 |
| RCC 9 | 0.8 | 0.9 |
| RCC 9 Margin | 0.5 | 0.6 |
| Normal Uterus | 0 | 0.4 |
| UtCa | 1.8 | 1.9 |
| Normal Thyroid | 1.4 | 1.8 |
| ThyCa 1 | 1.7 | 1.9 |
| ThyCa 2 | 0.9 | 0.9 |
| ThyCa 2 Margin | 1.5 | 1.5 |
| Normal Breast | 3.9 | 4.9 |
| BCa 1 | 19.8 | 26.2 |
| BCa 2 | 46.7 | 45.7 |
| BCa 3 Metastasis | 43.2 | 57.8 |
| BCa 4 Metastasis | 100 | 100 |
| BCa 5 | 2.4 | 2.7 |
| BCa 6 | 2.5 | 2.3 |
| BCa 7 | 41.2 | 45.7 |
| BCa 7 Margin | 5 | 5.7 |
| BCa 8 | 4 | 5.4 |
| BCa 8 Margin | 4.1 | 6.8 |
| Normal Liver | 0.2 | 0.3 |
| HCC 1 | 0.2 | 0.2 |
| HCC 2 | 0.2 | 0.1 |
| HCC 3 | 0.3 | 0.2 |
| HCC 4 | 0.2 | 0.2 |
| HCC 4 Margin | 0.5 | 0.5 |
| HCC 5 | 0.2 | 0.2 |
| HCC 5 Margin | 0 | 0 |
| Normal Bladder | 1.5 | 1.4 |
| TCC 1 | 0.3 | 0.2 |
| TCC 2 | 1.7 | 1.8 |
| TCC 3 | 3 | 3.3 |
| TCC 3 Margin | 2.9 | 1.4 |
| Normal Ovary | 0.3 | 0.3 |
| OVCa 1 | 3.3 | 3 |
| OVCa 2 | 3.1 | 3.1 |
| OVCa 2 Margin | 0.4 | 0.5 |
| Normal Stomach | 0.5 | 0.5 |
| GaCa 1 | 0.2 | 0.2 |
| GaCa 1 Margin | 0.4 | 0.4 |
| GaCa 2 | 0.8 | 0.7 |
| GaCa 2 Margin | 0.5 | 0.5 |
| GaCa 3 | 1 | 0.9 |
| GaCa 3 Margin | 0.1 | 0.1 |
| GaCa 4 | 1 | 0.8 |

### Example D2

### Antisense knockdown of LIV-1 mRNA in High-expressing and Low-expressing Tumor-derived Cell Lines

Five oligonucleotides were designed and synthesized as mixed-backbone oligonucleotides containing modified phosphorothioate segments at 5′ and 3′ ends and 2′-O-methyl RNA oligoribonucleotide segments located in the middle. The purity of the oligonucleotides was confirmed by Masspectrophometry. The oligonucleotide sequences for LIV-1 are: ASI: 5′ GATCCAGGCTCTGTCACTCAC 3′ (SEQ ID NO: 1606) (complementary to the sequence located in 5′ UTR of LIV-1). AS2: 5′ ATTATGGGATCAGCGTTC 3′ (SEQ ID NO: 1607) (complementary to the sequence surrounding ATG start codon). AS3: 5′ AGGCTTAAGATGACCACTRG 3′ (complementary to the sequence 3′ next to AS2). AS4: 5′ GTGGTGCTCGTIFCGAACAGT 3′ (SEQ ID NO: 1608) (complementary to the sequence in the middle of SLPI ORF). AS5: 5° CTGGGTCTTAGTCCGTGGCA 3′ (SEQ ID NO: 1609) (complementary to the sequence flanking the 3′ stop codon). 10,000 cells were seeded in each well of 96 well plate in compelete medium 24 hr before transfection to reach 50% confluency on the day of transfection. Oligonucleotides were diluted with Optimen to 100 and 400 nM, and mixed with Oligofectamine (Invitrogen) according to manufecture's instructions. Cells were washed with serum-free medium. The oligo and lipisome mixture were then added to cells. After 4 hr incubation period, serum were added back to cells. Readout assays were performed 24 and 48 hr after transfection. CellTiter 96Aqueous Non-Radioactive Cell Porliferation Assay Kit from Promega was used to determine the number of viable cells in proliferation assay. Briefly, 20 μl of combined MTS/PMS solution were diluted with 100 μl complete medium, and added to each well of the 96 well plate. After 1 hr incubation at 37° C., the absorbance at 490 nm was recorded using an ELISA plate reader.

FIG. 2. Antisense Knockdown of LIV-1 Inhibits T47D Cell Growth.

Breast cancer cell line T47D were transfected with 5 LIV-1 antisense oligonucleotides either individually or in the combinations as indicated. 72 hr after transfection, MTS assay was performed. The experiment was repeated twice with similar results. The representative data is shown. Asterisk indicates statistically significant difference on Student's 2-samples t test assuming unequal variances. P value for knockdown with AS4 comparing to scramble control at 400 nM is 0.000189.

FIG. 3. Antisense Knockdown of LIV-1 Onhibits Ovcar-4 Cell Growth.

Ovarian cancer cell line Ovcar4 were transfected with LWV-1 antisense oligonucleotides either individually or in the combinations as indicated. 72 hr after transfection, MTS assay was performed. Asterisk indicates statistically significant difference on Student's 2-samples t test assuming unequal variances. P value for knockdown with AS4 comparing to scramble control at 400 nM is 0.138366.

FIG. 4. RTOPCR Validation of Knockdown of LIV-1 Expression at mRNA Level in T47D Breast Cancer Cell Line.

T47D cells were transfected with LIV-1 antisense oligonucleotides in the combinations as indicated. 24 hr after transfection, cells were lysed in cell lysis buffer, and total RNA was isolated using RNAeasy kit from Qiagen. After DNAse digestion, cDNA was synthesized using Superscript II reverse transcriptase (BRL). RTQ-PCR analysis was performed according to the standard RTQPCR operation procedures.

### Other Embodiments

Although particular embodiments are disclosed herein in detail, this is done by way of example for purposes of illustration only, and is not intended to be limiting with respect to the scope of the appended claims, which follow. In particular, it is contemplated by the inventors that various substitutions, alterations, and modifications will be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims. The claims presented are representative of the inventions disclosed herein. Other, unclaimed inventions are also contemplated. Applicants reserve the right to pursue such inventions in later claims.

What is claimed is:

1. An isolated polypeptide comprising the mature form of an amino acid sequenced selected from the group consisting of SEQ ID NO:2n, wherein n is an integer between 1 and 606.

2. An isolated polypeptide comprising an amino acid sequence selected from the group consisting of SEQ ID NO:2n, wherein n is an integer between 1 and 606.

3. An isolated polypeptide comprising an amino acid sequence which is at least 95% identical to an amino acid sequence selected from the group consisting of SEQ ID NO:2n, wherein n is an integer between 1 and 606.

4. An isolated polypeptide, wherein the polypeptide comprises an amino acid sequence comprising one or more conservative substitutions in the amino acid sequence selected from the group consisting of SEQ ID NO:2n, wherein n is an integer between 1 and 606.

5. The polypeptide of claim 1 wherein said polypeptide is naturally occurring.

6. A composition comprising the polypeptide of claim 1 and a carrier.

7. A kit comprising, in one or more containers, the composition of claim 6.

8. The use of a therapeutic in the manufacture of a medicament for treating a syndrome associated with a human disease, the disease selected from a pathology associated with the polypeptide of claim 1, wherein the therapeutic comprises the polypeptide of claim 1.

9. A method for determining the presence or amount of the polypeptide of claim 1 in a sample, the method comprising:
(a) providing said sample;
(b) introducing said sample to an antibody that binds immunospecifically to the polypeptide; and
(c) determining the presence or amount of antibody bound to said polypeptide,
thereby determining the presence or amount of polypeptide in said sample.

10. A method for determining the presence of or predisposition to a disease associated with altered levels of expression of the polypeptide of claim 1 in a first mammalian subject, the method comprising:
a) measuring the level of expression of the polypeptide in a sample from the first mammalian subject; and
b) comparing the expression of said polypeptide in the sample of step (a) to the expression of the polypeptide present in a control sample from a second mammalian subject known not to have, or not to be predisposed to, said disease,
wherein an alteration in the level of expression of the polypeptide in the first subject as compared to the control sample indicates the presence of or predisposition to said disease.

11. A method of identifying an agent that binds to the polypeptide of claim 1, the method comprising:
(a) introducing said polypeptide to said agent; and
(b) determining whether said agent binds to said polypeptide.

12. The method of claim 11 wherein the agent is a cellular receptor or a downstream effector.

13. A method for identifying a potential therapeutic agent for use in treatment of a pathology, wherein the pathology is related to aberrant expression or aberrant physiological interactions of the polypeptide of claim 1, the method comprising:
(a) providing a cell expressing the polypeptide of claim 1 and having a property or function ascribable to the polypeptide;
(b) contacting the cell with a composition comprising a candidate substance; and
(c) determining whether the substance alters the property or function ascribable to the polypeptide;
whereby, if an alteration observed in the presence of the substance is not observed when the cell is contacted with a composition in the absence of the substance, the substance is identified as a potential therapeutic agent.

14. A method for screening for a modulator of activity of or of latency or predisposition to a pathology associated with the polypeptide of claim 1, said method comprising:
(a) administering a test compound to a test animal at increased risk for a pathology associated with the polypeptide of claim 1, wherein said test animal recombinantly expresses the polypeptide of claim 1;
(b) measuring the activity of said polypeptide in said test animal after administering the compound of step (a); and
(c) comparing the activity of said polypeptide in said test animal with the activity of said polypeptide. in a control animal not administered said polypeptide, wherein a change in the activity of said polypeptide in said test animal relative to said control animal indicates the test compound is a modulator activity of or latency or predisposition to, a pathology associated with the polypeptide of claim 1.

15. The method of claim 14, wherein said test animal is a recombinant test animal that expresses a test protein transgene or expresses said transgene under the control of a promoter at an increased level relative to a wild-type test animal, and wherein said promoter is not the native gene promoter of said transgene.

16. A method for modulating the activity of the polypeptide of claim 1, the method comprising contacting a cell sample expressing the polypeptide of claim 1 with a compound that binds to said polypeptide in an amount sufficient to modulate the activity of the polypeptide.

17. A method of treating or preventing a pathology associated with the polypeptide of claim 1, the method comprising administering the polypeptide of claim 1 to a subject in which such treatment or prevention is desired in an amount sufficient to treat or prevent the pathology in the subject.

18. The method of claim 17, wherein the subject is a human.

19. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising the amino acid sequence selected from the group consisting of SEQ ID NO:2n, wherein n is an integer between 1 and 606 or a biologically active fragment thereof.

20. An isolated nucleic acid molecule comprising a nucleic acid sequence selected from the group consisting of SEQ ID NO:2n−1, wherein n is an integer between 1 and 606.

21. The nucleic acid molecule of claim 20, wherein the nucleic acid molecule is naturally occurring.

22. A nucleic acid molecule, wherein the nucleic acid molecule differs by a single nucleotide from a nucleic acid sequence selected from the group consisting of SEQ ID NO: 2n−1, wherein n is an integer between 1 and 606.

23. An isolated nucleic acid molecule encoding the mature form of a polypeptide having an amino acid sequence selected from the group consisting of SEQ ID NO:2n, wherein n is an integer between 1 and 606.

24. An isolated nucleic acid molecule comprising a nucleic acid selected from the group consisting of 2n−1, wherein n is an integer between 1 and 606.

25. The nucleic acid molecule of claim 20, wherein said nucleic acid molecule hybridizes under stringent conditions to the nucleofide sequence selected from the group consisting of SEQ ID NO: 2n−1, wherein n is an integer between 1 and 606, or a complement of said nucleotide sequence.

26. A vector comprising the nucleic acid molecule of claim 20.

27. The vector of claim 26, further comprising a promoter operably linked to said nucleic acid molecule.

28. A cell comprising the vector of claim 26.

29. An antibody that immunospecifically binds to the polypeptide of claim 1.

30. The antibody of claim 29, wherein the antibody is a monoclonal antibody.

31. The antibody of claim 29, wherein the antibody is a humanized antibody.

32. A method for determining the presence or amount of the nucleic acid molecule of claim 20 in a sample, the method comprising:
(a) providing said sample;
(b) introducing said sample to a probe that binds to said nucleic acid molecule; and
(c) determining the presence or amount of said probe bound to said nucleic acid molecule,
thereby determining the presence or amount of the nucleic acid molecule in said sample.

33. The method of claim 32 wherein presence or amount of the nucleic acid molecule is used as a marker for cell or tissue type.

34. The method of claim 33 wherein the cell or tissue type is cancerous.

35. A method for determining the presence of or predisposition to a disease associated with altered levels of expression of the nucleic acid molecule of claim 20 in a first mammalian subject, the method comprising:
a) measuring the level of expression of the nucleic acid in a sample from the first mammalian subject; and
b) comparing the level of expression of said nucleic acid in the sample of step (a) to the level of expression of the nucleic acid present in a control sample from a second mammalian subject known not to have or not be predisposed to, the disease;
wherein an alteration in the level of expression of the nucleic acid in the first subject as compared to the control sample indicates the presence of or predisposition to the disease.

36. A method of producing the polypeptide of claim 1, the method comprising culturing a cell under conditions that lead to expression of the polypeptide, wherein said cell comprises a vector comprising an isolated nucleic acid molecule comprising a nucleic acid sequence selected from the group consisting of SEQ ID NO:2n−1, wherein n is an integer between 1 and 606.

37. The method of claim 36 wherein the cell is a bacterial cell.

38. The method of claim 36 wherein the cell is an insect cell.

39. The method of claim 36 wherein the cell is a yeast cell.

40. The method of claim 36 wherein the cell is a mammalian cell.

41. A method of producing the polypeptide of claim 2, the method comprising culturing a cell under conditions that lead to expression of the polypeptide, wherein said cell comprises a vector comprising an isolated nucleic acid molecule comprising a nucleic acid sequence selected from the group consisting of SEQ ID NO:2n−1, wherein n is an integer between 1 and 606.

42. The method of claim 41 wherein the cell is a bacterial cell.

43. The method of claim 41 wherein the cell is an insect cell.

44. The method of claim 41 wherein the cell is a yeast cell.

45. The method of claim 41 wherein the cell is a mammalian cell.
